(19) United States
(12) Patent Application Publication
(10) Pub. No.: US 2004/0072264 A1
(43) Pub. Date: Apr. 15, 2004

(54) Novel compositions and methods for cancer

(76) Inventors: David W. Morris, Davis, CA (US); Eric K. Engelhard, Davis, CA (US)

Correspondence Address: Shantanu Basu, Morrison & Foerster LLP, 755 Page Mill Road, Palo Alto, CA, 94304-1018, US

(21) Appl. No.: 10/052,482

(22) Filed: Nov. 8, 2001

Related U.S. Application Data
(63) Continuation-in-part of application No. 09/747,377, filed on Dec. 22, 2000.
(63) Continuation-in-part of application No. 09/798,586, filed on Mar. 2, 2001.

(51) Int. Cl. G01N 33/574; C12N 9/00; C07H 21/04; C12P 21/02

(52) U.S. Cl. 435/7.23; 536/23.2; 435/325; 435/320.1; 435/183

(57) ABSTRACT

The present invention relates to novel sequences for use in diagnosis and treatment of carcinomas, especially lymphoma carcinomas. In addition, the present invention describes the use of novel compositions for use in screening methods.

The present application is a continuing application of U.S. Ser. No. 09/747,377, filed Dec. 22, 2000 and Ser. No. 09/798,586, filed Mar. 2, 2001, both of which are expressly incorporated herein by reference.

### FIELD OF THE INVENTION

The present invention relates to novel sequences for use in diagnosis and treatment of cancer, especially carcinomas, as well as the use of the novel compositions in screening methods.

### BACKGROUND OF THE INVENTION

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes.

There are a number of viruses known to be involved in human cancer as well as in animal cancer. Of particular interest here are viruses that do not contain oncogenes themselves; these are slow-transforming retroviruses. They induce tumors by integrating into the host genome and affecting neighboring protooncogenes in a variety of ways, including promoter insertion, enhancer insertion, and/or truncation of a protooncogene or tumor suppressor gene. The analysis of sequences at or near the insertion sites led to the identification of a number of new protooncogenes.

With respect to lymphoma and leukemia, murine leukemia retrovirus (MuLV), such as SL3-3 or Akv, is a potent inducer of tumors when inoculated into susceptible newborn mice, or when carried in the germline. A number of sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al., J. Virology 67:7118 (1993); Joosten et al., Virology 268:308 (2000); and Li et al., Nature Genetics 23:348 (1999); all of which are expressly incorporated by reference herein.

Accordingly, it is an object of the invention to provide sequences involved in cancer and in particular in oncogenesis.

### SUMMARY OF THE INVENTION

In accordance with the objects outlined above, the present invention provides methods for screening for compositions which modulate carcinomas, especially lymphoma and leukemia. Also provided herein are methods of inhibiting proliferation of a cell, preferably a lymphoma cell. Methods of treatment of carcinomas, including diagnosis, are also provided herein.

In one aspect, a method of screening drug candidates comprises providing a cell that expresses a carcinoma associated (CA) gene or fragments thereof. Preferred embodiments of CA genes are genes which are differentially expressed in cancer cells, preferably lymphatic, breast, prostate or epithelial cells, compared to other cells. Preferred embodiments of CA genes used in the methods herein include, but are not limited to the nucleic acids selected from Tables 1-40. The method further includes adding a drug candidate to the cell and determining the effect of the drug candidate on the expression of the CA gene.

In one embodiment, the method of screening drug candidates includes comparing the level of expression in the absence of the drug candidate to the level of expression in the presence of the drug candidate.

Also provided herein is a method of screening for a bioactive agent capable of binding to a CA protein (CAP), the method comprising combining the CAP and a candidate bioactive agent, and determining the binding of the candidate agent to the CAP.

Further provided herein is a method for screening for a bioactive agent capable of modulating the 2@ activity of a CAP. In one embodiment, the method comprises combining the CAP and a candidate bioactive agent, and determining the effect of the candidate agent on the bioactivity of the CAP.

Also provided is a method of evaluating the effect of a candidate carcinoma drug comprising administering the drug to a patient and removing a cell sample from the patient. The expression profile of the cell is then determined. This method may further comprise comparing the expression profile of the patient to an expression profile of a heathy individual.

In a further aspect, a method for inhibiting the activity of an CA protein is provided. In one embodiment, the method comprises administering to a patient an inhibitor of a CA protein preferably selected from the group consisting of the sequences outlined in Tables 1-40 or their complements.

A method of neutralizing the effect of a CA protein, preferably a protein encoded by a nucleic acid selected from the group of sequences outlined in Tables 1-40, is also provided. Preferably, the method comprises contacting an agent specific for said protein with said protein in an amount sufficient to effect neutralization.

Moreover, provided herein is a biochip comprising a nucleic acid segment which encodes a CA protein, preferably selected from the sequences outlined in Tables 1-40.

Also provided herein is a method for diagnosing or determining the propensity to carcinomas, especially lymphoma or leukemia by sequencing at least one carcinoma or lymphoma gene of an individual. In yet another aspect of the invention, a method is provided for determining carcinoma including lymphoma and leukemia gene copy number in an individual.

Novel sequences are also provided herein. Other aspects of the invention will become apparent to the skilled artisan by the following description of the invention.

### DETAILED DESCRIPTION OF THE INVENTION

The present invention is directed to a number of sequences associated with carcinomas, especially lymphoma, breast cancer or prostate cancer. The relatively tight linkage between clonally-integrated proviruses and protooncogenes forms “provirus tagging”, in which slow-transforming retroviruses that act by an insertion mutation mechanism are used to isolate protooncogenes. In some models, uninfected animals have low cancer rates, and infected animals have high cancer rates. It is known that many of the retroviruses involved do not carry transduced host protooncogenes or pathogenic trans-acting viral genes, and thus the cancer incidence must therefor be a direct consequence of proviral integration effects into host protooncogenes. Since proviral integration is random, rare integrants will “activate” host protooncogenes that provide a selective growth advantage, and these rare events result in new proviruses at clonal stoichiometries in tumors.

The use of oncogenic retroviruses, whose sequences insert into the genome of the host organism resulting in carcinoma, allows the identification of host sequences involved in carcinoma. These sequences may then be used in a number of different ways, including diagnosis, prognosis, screening for modulators (including both agonists and antagonists), antibody generation (for immunotherapy and imaging), etc. However, as will be appreciated by those in the art, oncogenes that are identified in one type of cancer such as lymphoma or leukemia have a strong likelihood of being involved in other types of cancers as well. Thus, while the sequences outlined herein are initially identified as correlated with lymphoma, they can also be found in other types of cancers as well, outlined below.

Accordingly, the present invention provides nucleic acid and protein sequences that are associated with carcinoma, herein termed “carcinoma associated” or “CA” sequences. In a preferred embodiment, the present invention provides nulcleic acid and protein sequences that are associated with carcinomas which originate in lymphatic tissue, herein termed “lymphoma associated”, “leukemia associated” or “LA” sequences.

Suitable cancers which can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); cancers of the lymphomas (hodgkin's disease and non-hodgkin's lymphoma), multiple myeloma, and leukemias (lymphocytic leukemia; myeloid leukemia; monocytic leukemia; and other leukemias).

Other cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Branchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/ squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In addition, the genes may be involved in other diseases, such as but not limited to diseases associated with aging or neurodegenerative diseases.

Association in this context means that the nucleotide or protein sequences are either differentially expressed, activated, inactivated or altered in carcinomas as compared to normal tissue. As outlined below, CA sequences include those that are up-regulated (i.e. expressed at a higher level), as well as those that are down-regulated (i.e. expressed at a lower level), in carcinomas. CA sequences also include sequences which have been altered (i.e., truncated sequences or sequences with substitutions, deletions or insertions, including point mutations) and show either the same expression profile or an altered profile. In a preferred embodiment, the CA sequences are from humans; however, as will be appreciated by those in the art, CA sequences from other organisms may be useful in animal models of disease and drug evaluation; thus, other CA sequences are provided, from vertebrates, including mammals, including rodents (rats, mice, hamsters, guinea pigs, etc.), primates, farm animals (including sheep, goats, pigs, cows, horses, etc). In some cases, prokaryotic CA sequences may be useful. CA sequences from other organisms may be obtained using the techniques outlined below.

CA sequences can include both nucleic acid and amino acid sequences. In a preferred embodiment, the CA sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an CA protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the CA sequences are nucleic acids. As will be appreciated by those in the art and is more fully outlined below, CA sequences are useful in a variety of applications, including diagnostic applications, which will detect naturally occurring nucleic acids, as well as screening applications; for example, biochips comprising nucleic acid probes to the CA sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined below (for example in antisense applications or when a candidate agent is a nucleic acid), nucleic acid analogs may be used that have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805,(1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and by Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand “Watson” also defines the sequence of the other strand “Crick”; thus the sequences described herein also includes the complement of the sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

An CA sequence can be initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

The CA sequences of the invention were initially identified as described herein; basically, infection of mice with murine leukemia viruses (MLV) resulted in lymphoma, although many of these sequences will also be involved in other cancers as is generally outlined herein.

The CA sequences outlined herein comprise the insertion sites for the virus. In general, the retrovirus can cause carcinomas in three basic ways: first of all, by inserting upstream of a normally silent host gene and activating it (e.g. promoter insertion); secondly, by truncating a host gene that leads to oncogenesis; or by enhancing the transcription of a neighboring gene. For example, retrovirus enhancers, including SL3-3, are known to act on genes up to approximately 200 kilobases of the insertion site.

In a preferred embodiment, CA sequences are those that are up-regulated in carcinomas; that is, the expression of these genes is higher in carcinoma tissue as compared to normal tissue of the same differentiation stage. “Up-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are down-regulated in carcinomas; that is, the expression of these genes is lower in carcinoma tissue as compared to normal I tissue of the same differentiation stage. “Down-regulation” as used herein means at least about 50%, more preferably at least about 100%, more preferably at least about 150%, more preferably, at least about 200%, with from 300 to at least 1000% being especially preferred.

In a preferred embodiment, CA sequences are those that are altered but show either the same expression profile or an altered profile as compared to normal lymphoid tissue of the same differentiation stage. “Altered CA sequences” as used herein refers to sequences which are truncated, contain insertions or contain point mutations.

CA proteins of the present invention may be classified as secreted proteins, transmembrane proteins or intracellular proteins.

In a preferred embodiment the CA protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-213 homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In a preferred embodiment, the CA sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor, etc.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

CA proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In a preferred embodiment, the CA proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. CA proteins that are secreted proteins are particularly preferred in the present invention as they serve as good targets for diagnostic markers, for example for blood tests.

An CA sequence is initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

As used herein, a nucleic acid is a “CA nucleic acid” if the overall homology of the nucleic acid sequence to one of the nucleic acids of Tables 1-40 is preferably greater than about 75%, more preferably greater than about 80%, even more preferably greater than about 85% and most preferably greater than 90%. In some embodiments the homology will be as high as about 93 to 95 or 98%. In a preferred embodiment, the sequences which are used to determine sequence identity or similarity are selected from those of the nucleic acids of Tables 1-40. In another embodiment, the sequences are naturally occurring allelic variants of the sequences of the nucleic acids of Tables 1-40. In another embodiment, the sequences are sequence variants as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic acids of Tables 1-40. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acids of Tables 1-40, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, for example, nucleic acids which hybridize under high stringency to the nucleic acids identified in the figures, or their complements, are considered CA sequences. High stringency conditions are known in the art; see for example Maniatis et al., Molecular Cloning: A Laboratory Manual, 2d Edition, 1989, 2% and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

In addition, the CA nucleic acid sequences of the invention are fragments of larger genes, i.e. they are nucleic acid segments. Alternatively, the CA nucleic acid sequences can serve as indicators of oncogene position, for example, the CA sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the CA genes can be obtained, using techniques well known in the art for cloning either longer sequences or the full length sequences; see Maniatis et al., and Ausubel, et al., supra, hereby expressly incorporated by reference. In general, this is done using PCR, for example, kinetic PCR.

Once the CA nucleic acid is identified, it can be cloned and, if necessary, its constituent parts recombined to form the entire CA nucleic acid. Once isolated from its natural source, e.g., contained within a plasmid or other vector or excised therefrom as a linear nucleic acid segment, the recombinant CA nucleic acid can be further used as a probe to identify and isolate other CA nucleic acids, for example additional coding regions. It can also be used as a “precursor” nucleic acid to make modified or variant CA nucleic acids and proteins.

The CA nucleic acids of the present invention are used in several ways. In a first embodiment, nucleic acid probes to the CA nucleic acids are made and attached to biochips to be used in screening and diagnostic methods, as outlined below, or for administration, for example for gene therapy and/or antisense applications. Alternatively, the CA nucleic acids that include coding regions of CA proteins can be put into expression vectors for the expression of CA proteins, again either for screening purposes or for administration to a patient.

In a preferred embodiment, nucleic acid probes to CA nucleic acids (both the nucleic acid sequences outlined in the figures and/or the complements thereof) are made. The nucleic acid probes attached to the biochip are designed to be substantially complementary to the CA nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, resins, silica or silica-based materials including silicon and modified silicon, carbon, metals, inorganic glasses, etc. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affymetrix GeneChip technology.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, and an array of probe/primer reactions can simultaneously quantify the expression of multiple gene products of interest. See Germer, S., et al., Genome Res. 10:258-266 (2000); Heid, C. A., et al., Genome Res. 6, 986-994 (1996).

In a preferred embodiment, CA nucleic acids encoding CA proteins are used to make a variety of expression vectors to express CA proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the CA protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the CA protein; for example, transcriptional and translational regulatory nucleic acid sequences from Bacillus are preferably used to express the CA protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The CA proteins of the present invention are produced by culturing a host cell transformed with an expression vector containing nucleic acid encoding an CA protein, under the appropriate conditions to induce or cause expression of the CA protein. The conditions appropriate for CA protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the CA proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, CA proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the CA protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, CA proteins are produced in insect cells. Expression vectors for the transformation of insect cells, and in particular, baculovirus-based expression vectors, are well known in the art.

In a preferred embodiment, CA protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guilledimondii and P. pastoris, Schizosaccharomyces pombe, and Yarrowia lipolytica.

The CA protein may also be made as a fusion protein, using techniques well known in the art. Thus, for example, for the creation of monoclonal antibodies. If the desired epitope is small, the CA protein may be fused to a carrier protein to form an immunogen. Alternatively, the CA protein may be made as a fusion protein to increase expression, or for other reasons. For example, when the CA protein is an CA peptide, the nucleic acid encoding the peptide may be linked to other nucleic acid for expression purposes.

In one embodiment, the CA nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or X antigens; and c) colored or fluorescent dyes. The labels may be incorporated into the CA nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides CA protein sequences. An CA protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the CA protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of CA proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques known in the art as are outlined above for the nucleic acid homologies.

CA proteins of the present invention may be shorter or longer than the wild type amino acid sequences. Thus, in a preferred embodiment, included within the definition of CA proteins are portions or fragments of the wild type sequences herein. In addition, as outlined above, the CA nucleic acids of the invention may be used to obtain additional coding regions, and thus additional protein sequence, using techniques known in the art.

In a preferred embodiment, the CA proteins are derivative or variant CA proteins as compared to the wild-type sequence. That is, as outlined more fully below, the derivative CA peptide will contain at least one amino acid substitution, deletion or insertion, with amino acid substitutions being particularly preferred. The amino acid substitution, insertion or deletion may occur at any residue within the CA peptide.

Also included in an embodiment of CA proteins of the present invention are amino acid sequence variants. These variants fall into one or more of three classes: substitutional, insertional or deletional JEW variants. These variants ordinarily are prepared by site specific mutagenesis of nucleotides in the DNA encoding the CA protein, using cassette or PCR mutagenesis or other techniques well known in the art, to produce DNA encoding the variant, and thereafter expressing the DNA in recombinant cell culture as outlined above. However, variant CA protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the CA protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed CA variants screened for the optimal combination of desired activity. Techniques for making substitution mutations at predetermined sites in DNA having a known sequence are well known, for example, M13 primer mutagenesis and LAR mutagenesis. Screening of the mutants is done using assays of CA protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the CA protein are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart 1. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the CA proteins as needed. Alternatively, the variant may be designed such that the biological activity of the CA protein is altered. For example, glycosylation sites may be altered or removed, dominant negative mutations created, etc.

Covalent modifications of CA polypeptides are included within the scope of this invention, for example for use in screening. One type of covalent modification includes reacting targeted amino acid residues of an CA polypeptide with an organic derivatizing agent that is capable of reacting with selected side chains or the N-or C-terminal residues of an CA polypeptide. Derivatization with bifunctional agents is useful, for instance, for crosslinking CA polypeptides to a water-insoluble support matrix or surface for use in the method for purifying anti-CA antibodies or screening assays, as is more fully described below. Commonly used crosslinking agents include, e.g., 1 ,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the a-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the CA polypeptide included within the scope of this invention comprises altering the native glycosylation pattern of the polypeptide. “Altering the native glycosylation pattern” is intended for purposes herein to mean deleting one or more carbohydrate moieties found in native sequence CA polypeptide, and/or adding one or more glycosylation sites that are not present in the native sequence CA polypeptide.

Addition of glycosylation sites to CA polypeptides may be accomplished by altering the amino acid sequence thereof. The alteration may be made, for example, by the addition of, or substitution by, one or more serine or threonine residues to the native sequence CA polypeptide (for O-linked glycosylation sites). The CA amino acid sequence may optionally be altered through changes at the DNA level, particularly by mutating the DNA encoding the CA polypeptide at preselected bases such that codons are generated that will translate into the desired amino acids.

Another means of increasing the number of carbohydrate moieties on the CA polypeptide is by chemical or enzymatic coupling of glycosides to the polypeptide. Such methods are described in the art, e.g., in WO 87/05330 published Sep. 11, 1987, and in Aplin and Wriston, LA Crit. Rev. Biochem., pp. 259-306 (1981).

Removal of carbohydrate moieties present on the CA polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of CA comprises linking the CA polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

CA polypeptides of the present invention may also be modified in a way to form chimeric molecules comprising an CA polypeptide fused to another, heterologous polypeptide or amino acid sequence. In one embodiment, such a chimeric molecule comprises a fusion of an CA polypeptide with a tag polypeptide which provides an epitope to which an anti-tag antibody can selectively bind. The epitope tag is generally placed at the amino-or carboxyl-terminus of the CA polypeptide, although internal fusions may also be tolerated in some instances. The presence of such epitope-tagged forms of an CA polypeptide can be detected using an antibody against the tag polypeptide. Also, provision of the epitope tag enables the CA polypeptide to be readily purified by affinity purification using an anti-tag antibody or another type of affinity matrix that binds to the epitope tag. In an alternative embodiment, the chimeric molecule may comprise a fusion of an CA polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of CA protein in one embodiment are other CA proteins of the CA family, and CA proteins from other organisms, which are cloned and expressed as outlined below. Thus, probe or degenerate polymerase chain reaction (PCR) primer sequences may be used to find other related CA proteins from humans or other organisms. As will be appreciated by those in the art, particularly useful probe and/or PCR primer sequences include the unique areas of the CA nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, CA proteins can be made that are longer than those encoded by the nucleic acids of the figures, for example, by the elucidation of additional sequences, the addition of epitope or purification tags, the addition of other fusion sequences, etc.

CA proteins may also be identified as being encoded by CA nucleic acids. Thus, CA proteins are encoded by nucleic acids that will hybridize to the sequences of the sequence listings, or their complements, as outlined herein.

In a preferred embodiment, the invention provides CA antibodies. In a preferred embodiment, when the CA protein is to be used to generate antibodies, for example for immunotherapy, the CA protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller CA protein will be able to bind to the full length protein. In a preferred embodiment, the epitope is unique; that is, antibodies generated to a unique epitope show little or no cross-reactivity.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-40, or fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens. In the present case, one of the binding specificities is for a protein encoded by a nucleic acid of Tables 1-40, or a fragment thereof, the other one is for any other antigen, and preferably for a cell-surface protein or receptor or receptor subunit, preferably one that is tumor specific.

In a preferred embodiment, the antibodies to CA are capable of reducing or eliminating the biological function of CA, as is described below. That is, the addition of anti-CA antibodies (either polyclonal or preferably monoclonal) to CA (or cells containing CA) may reduce or eliminate the CA activity. Generally, at least a 25% decrease in activity is preferred, with at least about 50% being particularly preferred and about a 95-100% decrease being especially preferred.

In a preferred embodiment the antibodies to the CA proteins are humanized antibodies. Humanized forms of non-human (e.g., murine) antibodies are chimeric molecules of immunoglobulins, immunoglobulin chains or fragments thereof (such as Fv, Fab, Fab′, F(ab′)₂ or other antigen binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human.

These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of a carcinoma with an antibody raised against an CA protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment, oncogenes which encode secreted growth factors may be inhibited by raising antibodies against CA proteins that are secreted proteins as described above. Without being bound by theory, antibodies used for treatment, bind and prevent the secreted protein from binding to its receptor, thereby inactivating the secreted CA protein.

In another preferred embodiment, the CA protein to which antibodies are raised is a transmembrane protein. Without being bound by theory, antibodies used for treatment, bind the extracellular domain of the CA protein and prevent it from binding to other proteins, such as circulating ligands or cell-associated molecules. The antibody may cause down-regulation of the transmembrane CA protein. As will be appreciated by one of ordinary skill in the art, the antibody may be a competitive, non-competitive or uncompetitive inhibitor of protein binding to the extracellular domain of the CA protein. The antibody is also an antagonist of the CA protein. Further, the antibody prevents activation of the transmembrane CA protein. In one aspect, when the antibody prevents the binding of other molecules to the CA protein, the antibody prevents growth of the cell. The antibody may also sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, carcinomas may be treated by administering to a patient antibodies directed against the transmembrane CA protein.

In another preferred embodiment, the antibody is conjugated to a therapeutic moiety. In one aspect the therapeutic moiety is a small molecule that modulates the activity of the CA protein. In another aspect the therapeutic moiety modulates the activity of molecules associated with or in close proximity to the CA protein. The therapeutic moiety may inhibit enzymatic activity such as protease or protein kinase activity associated with carcinoma.

In a preferred embodiment, the therapeutic moiety may also be a cytotoxic agent. In this method, targeting the cytotoxic agent to tumor tissue or cells, results in a reduction in the number of afflicted cells, thereby reducing symptoms associated with carcinomas, including lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against CA proteins, or binding of a radionuclide to a chelating agent that has been covalently attached to the antibody. Targeting the therapeutic moiety to transmembrane CA proteins not only serves to increase the local concentration of therapeutic moiety in the carcinoma of interest, i.e., lymphoma, but also serves to reduce deleterious side effects that may be associated with the therapeutic moiety.

In another preferred embodiment, the CA protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the CA protein can be targeted within a cell, i.e., the nucleus, an antibody thereto contains a signal for that target localization, i.e., a nuclear localization signal.

The CA antibodies of the invention specifically bind to CA proteins. By “specifically bind” herein is meant that the antibodies bind to the protein with a binding constant in the range of at least 10⁻⁴-10⁻⁶ M⁻¹, with a preferred range being 10⁻⁷-10⁻⁹ M⁻¹.

In a preferred embodiment, the CA protein is purified or isolated after expression. CA proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the CA protein may be purified using a standard anti-CA antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the CA protein. In some instances no purification will be necessary.

Once expressed and purified if necessary, the CA proteins and nucleic acids are useful in a number of applications.

In one aspect, the expression levels of genes are determined for different cellular states in the carcinoma phenotype; that is, the expression levels of genes in normal tissue and in carcinoma tissue (and in some cases, for varying severities of lymphoma that relate to prognosis, as outlined below) are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or carcinoma tissue.

“Differential expression,” or grammatical equivalents as used herein, refers to both qualitative as well as quantitative differences in the genes temporal and/or cellular expression patterns within and among the cells. Thus, a differentially expressed gene can qualitatively have its expression altered, including an activation or inactivation, in, for example, normal versus carcinoma tissue. That is, genes may be turned on or turned off in a particular state, relative to another state. As is apparent to the skilled hi artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip® expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the CA protein and standard immunoassays (ELISAs, etc.) or other techniques, including mass spectroscopy assays, 2D gel electrophoresis assays, etc. Thus, the proteins corresponding to CA genes, i.e. those identified as being important in a particular carcinoma phenotype, i.e., lymphoma, can be evaluated in a diagnostic test specific for that carcinoma.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the CA nucleic acid probes may be attached to biochips as outlined herein for the detection and quantification of CA sequences in a particular cell. The assays are done as is known in the art. As will be appreciated by those in the art, any number of different CA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect CA sequences that are up-regulated or down-regulated in carcinomas as compared to normal tissue. In instances where the CA sequence has been altered but shows the same expression profile or an altered expression profile, the protein will be detected as outlined herein.

In a preferred embodiment nucleic acids encoding the CA protein are detected. Although DNA or RNA encoding the CA protein may be detected, of particular interest are methods wherein the mRNA encoding a CA protein is detected. The presence of mRNA in a sample is an indication that the CA gene has been transcribed to form the mRNA, and suggests that the protein is expressed. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a CA protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The CA proteins, antibodies, nucleic acids, modified proteins and cells containing CA sequences are used in diagnostic assays. This can be done on an individual gene or corresponding polypeptide level, or as sets of assays.

As described and defined herein, CA proteins find use as markers of carcinomas, including lymphomas such as, but not limited to, Hodgkin's and non-Hodgkin lymphoma. Detection of these proteins in putative carcinoma tissue or patients allows for a determination or diagnosis of the type of carcinoma. Numerous methods known to those of ordinary skill in the art find use in detecting carcinomas. In one embodiment, antibodies are used to detect CA proteins. A preferred method separates proteins from a sample or patient by electrophoresis on a gel (typically a denaturing and reducing protein gel, but may be any other type of gel including isoelectric focusing gels and the like). Following separation of proteins, the CA protein is detected by immunoblotting with antibodies raised against the CA protein. Methods of immunoblotting are well known to those of ordinary skill in the art.

In another preferred method, antibodies to the CA protein find use in in situ imaging techniques. In this method cells are contacted with from one to many antibodies to the CA protein(s). Following washing to remove non-specific antibody binding, the presence of the antibody or antibodies is detected. In one embodiment the antibody is detected by incubating with a secondary antibody that contains a detectable label. In another method the primary antibody to the CA protein(s) contains a detectable label. In another preferred embodiment each one of multiple primary antibodies contains a distinct and detectable label. This method finds particular use in simultaneous screening for a plurality of CA proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing carcinomas from blood samples. As previously described, certain CA proteins are secreted/circulating molecules. Blood samples, therefore, are useful as samples to be probed or tested for the presence of secreted CA proteins. Antibodies can be used to detect the CA proteins by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled CA nucleic acid probes to tissue arrays is done. For example, arrays of tissue samples, including CA tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes which indicate the diagnosis may differ from those which indicate the prognosis.

In a preferred embodiment, the CA proteins, antibodies, nucleic acids, modified proteins and cells containing CA sequences are used in prognosis assays. As above, gene expression profiles can be generated that correlate to carcinoma, especially lymphoma, severity, in terms of long term prognosis. Again, this may be done on either a protein or gene level, with the use of genes being preferred. As above, the CA probes are attached to biochips for the detection and quantification of CA sequences in a tissue or patient. The assays proceed as outlined for diagnosis.

In a preferred embodiment, any of the CA sequences as described herein are used in drug screening assays. The CA proteins, antibodies, nucleic acids, modified proteins and cells containing CA sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In a preferred embodiment, the CA proteins, antibodies, nucleic acids, modified proteins and cells containing the native or modified CA proteins are used in screening assays. That is, the present invention provides novel methods for screening for compositions which modulate the carcinoma phenotype. As above, this can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the CA genes herein, a variety of assays to evaluate the effects of agents on gene expression may be executed. In a preferred embodiment, assays may be run on an individual gene or protein level. That is, having identified a particular gene as aberrantly regulated in carcinoma, candidate bioactive agents may be screened to modulate the genes response. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the CA sequence has been altered but shows the same expression profile or an altered expression profile, the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the CA protein and standard immunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the CA nucleic acid probes are attached to biochips as outlined herein for the detection and quantification of CA sequences in a particular cell. The assays are further described below.

Generally, in a preferred embodiment, a candidate bioactive agent is added to the cells prior to analysis. Moreover, screens are provided to identify a candidate bioactive agent which modulates a particular type of carcinoma, modulates CA proteins, binds to a CA protein, or interferes between the binding of a CA protein and an antibody.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the carcinoma phenotype, binding to and/or modulating the bioactivity of an CA protein, or the expression of a CA sequence, including both nucleic acid sequences and protein sequences. In a particularly preferred embodiment, the candidate agent suppresses a CA phenotype, for example to a normal tissue fingerprint. Similarly, the candidate agent preferably suppresses a severe CA phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of an CA protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups.

The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration.

If non-naturally occurring side chains are used, non-amino acid substituents may be used, for example to prevent or retard in vivo degradations. In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids, as defined above.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

In assays for altering the expression profile of one or more CA genes, after the candidate agent has been added and the cells allowed to incubate for some period of time, the sample containing the target sequences to be analyzed is added to the biochip. If required, the target sequence is prepared using known techniques. For example, the sample may be treated to lyse the cells, using known lysis buffers, electroporation, etc., with purification and/or amplification such as PCR occurring as needed, as will be appreciated by those in the art. For example, an in vitro transcription with labels covalently attached to the nucleosides is done. Generally, the nucleic acids are labeled with a label as defined herein, with biotin-FITC or PE, cy3 and cy5 being particularly preferred.

In a preferred embodiment, the target sequence is labeled with, for example, a fluorescent, chemiluminescent, chemical, or radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin.

For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data is analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to alter the expression of the genes individually. That is, screening for modulation of regulation of expression of a single gene can be done. Thus, for example, particularly in the case of target genes whose presence or absence is unique between two states, screening is done for modulators of the target gene expression.

In addition, screens can be done for novel genes that are induced in response to a candidate agent. After identifying a candidate agent based upon its ability to suppress a CA expression pattern leading to a normal expression pattern, or modulate a single CA gene expression profile so as to mimic the expression of the gene from normal tissue, a screen as described above can be performed to identify genes that are specifically modulated in response to the agent. Comparing expression profiles between normal tissue and agent treated CA tissue reveals genes that are not expressed in normal tissue or CA tissue, but are expressed in agent treated tissue. These agent specific sequences can be identified and used by any of the methods described herein for CA genes or proteins. In particular these sequences and the proteins they encode find use in marking or identifying agent treated cells. In addition, antibodies can be raised against the agent induced proteins and used to target novel therapeutics to the treated CA tissue sample.

Thus, in one embodiment, a candidate agent is administered to a population of CA cells, that thus has an associated CA expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, CA tissue may be screened for agents that reduce or suppress the CA phenotype. A change in at least one gene of the expression profile indicates that the agent has an effect on CA activity. By defining such a signature for the CA phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “CA proteins” or an “CAP”. The CAP may be a fragment, or alternatively, be the full length protein to the fragment encoded by the nucleic acids of Tables 1-40. Preferably, the CAP is a fragment. In another embodiment, the sequences are sequence variants as further described herein.

Preferably, the CAP is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the c-terminus of the fragment is kept as a free acid and the n-terminus is a free amine to aid in coupling, i.e., to cysteine.

In one embodiment the CA proteins are conjugated to an immunogenic agent as discussed herein. In one embodiment the CA protein is conjugated to BSA.

In a preferred embodiment, screening is done to alter the biological function of the expression product of the CA gene. Again, having identified the importance of a gene in a particular state, screening for agents that bind and/or modulate the biological activity of the gene product can be run as is more fully outlined below.

In a preferred embodiment, screens are designed to first find candidate agents that can bind to CA proteins, and then these agents may be used in assays that evaluate the ability of the candidate agent to modulate the CAP activity and the carcinoma phenotype. Thus, as will be appreciated by those in the art, there are a number of different assays which may be run; binding assays and activity assays.

In a preferred embodiment, binding assays are done. In general, purified or isolated gene product is used; that is, the gene products of one or more CA nucleic acids are made. In general, this is done as is known in the art. For example, antibodies are generated to the protein gene products, and standard immunoassays are run to determine the amount of protein present. Alternatively, cells comprising the CA proteins can be used in the assays.

Thus, in a preferred embodiment, the methods comprise combining a CA protein and a candidate bioactive agent, and determining the binding of the candidate agent to the CA protein. Preferred embodiments utilize the human or mouse CA protein, although other mammalian proteins may also be used, for example for the development of animal models of human disease. In some embodiments, as outlined herein, variant or derivative CA proteins may be used.

Generally, in a preferred embodiment of the methods herein, the CA protein or the candidate agent is non-diffusably bound to an insoluble support having isolated sample receiving areas (e.g. a microtiter plate, an array, etc.). The insoluble supports may be made of any composition to which the A/ compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the CA protein is bound to the support, and a candidate bioactive agent is added to the assay. Alternatively, the candidate agent is bound to the support and the CA protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the CA protein may be done in a number of ways. In a preferred embodiment, the candidate bioactive agent is labeled, and binding determined directly. For example, this may be done by attaching all or a portion of the CA protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using ¹²⁵I for the proteins, for example, and a fluorophor for the candidate agents.

In a preferred embodiment, the binding of the candidate bioactive agent is determined through the use of competitive binding assays. In this embodiment, the competitor is a binding moiety known to bind to the target molecule (i.e. CA protein), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present.

Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the CA protein and thus is capable of binding to, and potentially modulating, the activity of the CA protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the CA protein with a higher affinity. Thus, if the candidate bioactive agent is labeled, the presence of the label on the support, coupled with a lack of competitor binding, may indicate that the candidate agent is capable of binding to the CA protein.

In a preferred embodiment, the methods comprise differential screening to identity bioactive agents that are capable of modulating the activity of the CA proteins. In this embodiment, the methods comprise combining a CA protein and a competitor in a first sample. A second sample comprises a candidate bioactive agent, a CA protein and a competitor. The binding of the competitor is determined for both samples, and a change, or difference in binding between the two samples indicates the presence of an agent capable of binding to the CA protein and potentially modulating its activity. That is, if the binding of the competitor is different in the second sample relative to the first sample, the agent is capable of binding to the CA protein.

Alternatively, a preferred embodiment utilizes differential screening to identify drug candidates that bind to the native CA protein, but cannot bind to modified CA proteins. The structure of the CA protein may be modeled, and used in rational drug design to synthesize agents that interact with that site. Drug candidates that affect CA bioactivity are also identified by screening drugs for the ability to either enhance or reduce the activity of the protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of CA proteins may also be done. In a preferred embodiment, methods for screening for a bioactive agent capable of modulating the activity of CA proteins comprise the steps of adding a candidate bioactive agent to a sample of CA proteins, as above, and determining an alteration in the biological activity of CA proteins. “Modulating the activity of an CA protein” includes an increase in activity, a decrease in activity, or a change in the type or kind of activity present. Thus, in this embodiment, the candidate agent should both bind to CA proteins (although this may not be necessary), and alter its biological or biochemical activity as defined herein. The methods include both in vitro screening methods, as are generally outlined above, and in vivo screening of cells for alterations in the presence, distribution, activity or amount of CA proteins.

Thus, in this embodiment, the methods comprise combining a CA sample and a candidate bioactive agent, and evaluating the effect on CA activity. By “CA activity” or grammatical equivalents herein is meant one of the CA protein's biological activities, including, but not limited to, its role in tumorigenesis, including cell division, preferably in lymphatic tissue, cell proliferation, tumor growth and transformation of cells. In one embodiment, CA activity includes activation of or by a protein encoded by a nucleic acid of Tables 1-40. An inhibitor of CA activity is the inhibition of any one or more CA activities.

In a preferred embodiment, the activity of the CA protein is increased; in another preferred embodiment, the activity of the CA protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a CA protein. The methods comprise adding a candidate bioactive agent, as defined above, to a cell comprising CA proteins. Preferred cell types include almost any cell. The cells contain a recombinant nucleic acid that encodes a CA protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the CA protein.

In one embodiment, a method of inhibiting carcinoma cancer cell division, is provided. The method comprises administration of a carcinoma cancer inhibitor.

In a preferred embodiment, a method of inhibiting lymphoma carcinoma cell division is provided comprising administration of a lymphoma carcinoma inhibitor.

In another embodiment, a method of inhibiting tumor growth is provided. The method comprises administration of a carcinoma cancer inhibitor. In a particularly preferred embodiment, a method of inhibiting tumor growth in lymphatic tissue is provided comprising administration of a lymphoma inhibitor.

In a further embodiment, methods of treating cells or individuals with cancer are provided. The method comprises administration of a carcinoma cancer inhibitor. Preferably, the carcinoma is a lymphoma carcinoma.

In one embodiment, a carcinoma cancer inhibitor is an antibody as discussed above. In another embodiment, the carcinoma cancer inhibitor is an antisense molecule. Antisense molecules as used herein include antisense or sense oligonucleotides comprising a singe-stranded nucleic acid sequence (either RNA or DNA) capable of binding to target mRNA (sense) or DNA (antisense) sequences for carcinoma cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. The agents may be administered alone or in combination with other treatments, i.e., radiation.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Without being bound by theory, it appears that the various CA sequences are important in carcinomas. Accordingly, disorders based on mutant or variant CA genes may be determined. In one embodiment, the invention provides methods for identifying cells containing variant CA genes comprising determining all or part of the sequence of at least one endogenous CA genes in a cell. As will be appreciated by those in the art, this may be done using any number of sequencing techniques. In a preferred embodiment, the invention provides methods of identifying the CA genotype of an individual comprising determining all or part of the sequence of at least one CA gene of the individual. This is generally done in at least one tissue of the individual, and may include the evaluation of a number of tissues or different samples of the same tissue. The method may include comparing the sequence of the sequenced CA gene to a known CA gene, i.e., a wild-type gene. As will be appreciated by those in the art, alterations in the sequence of some oncogenes can be an indication of either the presence of the disease, or propensity to develop the disease, or prognosis evaluations.

The sequence of all or part of the CA gene can then be compared to the sequence of a known CA gene to determine if any differences exist. This can be done using any number of known homology programs, such as Bestfit, etc. In a preferred embodiment, the presence of a difference in the sequence between the CA gene of the patient and the known CA gene is indicative of a disease state or a propensity for a disease state, as outlined herein.

In a preferred embodiment, the CA genes are used as probes to determine the number of copies of the CA gene in the genome. For example, some cancers exhibit chromosomal deletions or insertions, resulting in an alteration in the copy number of a gene.

In another preferred embodiment CA genes are used as probes to determine the chromosomal location of the CA genes. Information such as chromosomal location finds use in providing a diagnosis or prognosis in particular when chromosomal abnormalities such as translocations, and the like are identified in CA gene loci.

Thus, in one embodiment, methods of modulating CA in cells or organisms are provided. In one embodiment, the methods comprise administering to a cell an anti-CA antibody that reduces or eliminates the biological activity of an endogenous CA protein. Alternatively, the methods comprise administering to a cell or organism a recombinant nucleic acid encoding a CA protein. As will be appreciated by those in the art, this may be accomplished in any number of ways. In a preferred embodiment, for example when the CA sequence is down-regulated in carcinoma, the activity of the CA gene is increased by increasing the amount of CA in the cell, for example by overexpressing the art endogenous CA or by administering a gene encoding the CA sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the CA sequence is up-regulated in carcinoma, the activity of the endogenous CA gene is decreased, for example by the administration of a CA antisense nucleic acid.

In one embodiment, the CA proteins of the present invention may be used to generate polyclonal and monoclonal antibodies to CA proteins, which are useful as described herein. Similarly, the CA proteins can be coupled, using standard technology, to affinity chromatography columns. These columns may then be used to purify CA antibodies. In a preferred embodiment, the antibodies are generated to epitopes unique to a CA protein; that is, the antibodies show little or no cross-reactivity to other proteins. These antibodies find use in a number of applications. For example, the CA antibodies may be coupled to standard affinity chromatography columns and used to purify CA proteins. The antibodies may also be used as blocking polypeptides, as outlined above, since they will specifically bind to the CA protein.

In one embodiment, a therapeutically effective dose of a CA or modulator thereof is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for CA degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The administration of the CA proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the CA proteins and modulators may be directly applied as a solution or spray.

The pharmaceutical compositions of the present invention comprise a CA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

In a preferred embodiment, CA proteins and modulators are administered as therapeutic agents, and can be formulated as outlined above. Similarly, CA genes (including both the full-length sequence, partial sequences, or regulatory sequences of the CA coding regions) can be administered in gene therapy applications, as is known in the art. These CA genes can include antisense applications, either as gene therapy (i.e. for incorporation into the genome) or as antisense compositions, as will be appreciated by those in the art.

In a preferred embodiment, CA genes are administered as DNA vaccines, either single genes or combinations of CA genes. Naked DNA vaccines are generally known in the art. Brower, Nature Biotechnology, 16:1304-1305 (1998).

In one embodiment, CA genes of the present invention are used as DNA vaccines. Methods for the use of genes as DNA vaccines are well known to one of ordinary skill in the art, and include placing a CA gene or portion of a CA gene under the control of a promoter for expression in a patient with carcinoma. The CA gene used for DNA vaccines can encode full-length CA proteins, but more preferably encodes portions of the CA proteins including peptides derived from the CA protein. In a preferred embodiment a patient is immunized with a DNA vaccine comprising a plurality of nucleotide sequences derived from a CA gene. Similarly, it is possible to immunize a patient with a plurality of CA genes or portions thereof as defined herein. Without being bound by theory, expression of the polypeptide encoded by the DNA vaccine, cytotoxic T-cells, helper T-cells and antibodies are induced which recognize and destroy or eliminate cells expressing CA proteins.

In a preferred embodiment, the DNA vaccines include a gene encoding an adjuvant molecule with the DNA vaccine. Such adjuvant molecules include cytokines that increase the immunogenic response to the CA polypeptide encoded by the DNA vaccine. Additional or alternative adjuvants are known to those of ordinary skill in the art and find use in the invention.

In another preferred embodiment CA genes find use in generating animal models of carcinomas, particularly lymphoma carcinomas. As is appreciated by one of ordinary skill in the art, when the CA gene identified is repressed or diminished in CA tissue, gene therapy technology wherein antisense RNA directed to the CA gene will also diminish or repress expression of the gene. An animal generated as such serves as an animal model of CA that finds use in screening bioactive drug candidates. Similarly, gene knockout technology, for example as a result of homologous recombination with an appropriate gene targeting vector, will result in the absence of the CA protein. When desired, tissue-specific expression or knockout of the CA protein may be necessary.

It is also possible that the CA protein is overexpressed in carcinoma. As such, transgenic animals can be generated that overexpress the CA protein. Depending on the desired expression level, promoters of various strengths can be employed to express the transgene. Also, the number of copies of the integrated transgene can be determined and compared for a determination of the expression level of the transgene. Animals generated by such methods find use as animal models of CA and are additionally useful in screening for bioactive molecules to treat carcinoma.

The CA nucleic acid sequences of the invention are depicted in Tables 1-40. The sequences in each Table include genomic sequence, mRNA and coding sequences for both mouse and human. The different sequences are assigned the following SEQ ID Nos:

TABLE 1

(mouse gene: Myc; human gene MYC)
Mouse genomic sequence (SEQ ID NO:1)
Mouse mRNA sequence (SEQ ID NO:2)
Mouse coding sequence (SEQ ID NO:3)
Human genomic sequence (SEQ ID NO:4)
Human mRNA sequence (SEQ ID NO:5)
Human coding sequence (SEQ ID NO:6)

TABLE 2

(mouse gene Bach2; human gene BACH2)
Mouse genomic sequence (SEQ ID NO:7)
Mouse mRNA sequence (SEQ ID NO:8)
Mouse coding sequence (SEQ ID NO:9)
Human genomic sequence (SEQ ID NO:10)
Human mRNA sequence (SEQ ID NO:11)
Human coding sequence (SEQ ID NO:12)

TABLE 3

(mouse gene Wnt1; human gene WNT1)
Mouse genomic sequence (SEQ ID NO:13)
Mouse mRNA sequence (SEQ ID NO:14)
Mouse coding sequence (SEQ ID NO:15)
Human genomic sequence (SEQ ID NO:16)
Human mRNA sequence (SEQ ID NO:17)
Human coding sequence (SEQ ID NO:18)

TABLE 4

(mouse gene Rasgrp1; human gene: RASGRP1)
Mouse genomic sequence (SEQ ID NO:19)
Mouse mRNA sequence (SEQ ID NO:20)
Mouse coding sequence (SEQ ID NO:21)
Human genomic sequence (SEQ ID NO:22)
Human mRNA sequence (SEQ ID NO:23)
Human coding sequence (SEQ ID NO:24)

TABLE 5

(mouse gene: Nmyc1; human gene: MYCN)
Mouse genomic sequence (SEQ ID NO:25)
Mouse mRNA sequence (SEQ ID NO:26)
Mouse coding sequence (SEQ ID NO:27)
Human genomic sequence (SEQ ID NO:28)
Human mRNA sequence (SEQ ID NO:29)
Human coding sequence (SEQ ID NO:30)

TABLE 6

(mouse gene: Myb; human gene: MYB)
Mouse genomic sequence (SEQ ID NO:31)
Mouse mRNA sequence (SEQ ID NO:32)
Mouse coding sequence (SEQ ID NO:33)
Human genomic sequence (SEQ ID NO:34)
Human mRNA sequence (SEQ ID NO:35)
Human coding sequence (SEQ ID NO:36)

TABLE 7

(mouse gene: Sox4; human gene: SOX4)
Mouse genomic sequence (SEQ ID NO:37)
Mouse mRNA sequence (SEQ ID NO:38)
Mouse coding sequence (SEQ ID NO:39)
Human genomic sequence (SEQ ID NO:40)
Human mRNA sequence (SEQ ID NO:41)
Human coding sequence (SEQ ID NO:42)

TABLE 8

(mouse gene: Tcof1; human gene: TCOF1)
Mouse genomic sequence (SEQ ID NO:43)
Mouse mRNA sequence (SEQ ID NO:44)
Mouse coding sequence (SEQ ID NO:45)
Human genomic sequence (SEQ ID NO:46)
Human mRNA sequence (SEQ ID NO:47)
Human coding sequence (SEQ ID NO:48)

TABLE 9

(mouse gene: Pim1; human gene: PIM1)
Mouse genomic sequence (SEQ ID NO:49)
Mouse mRNA sequence (SEQ ID NO:50)
Mouse coding sequence (SEQ ID NO:51)
Human genomic sequence (SEQ ID NO:52)
Human mRNA sequence (SEQ ID NO:53)
Human coding sequence (SEQ ID NO:54)

TABLE 10

(mouse gene: Wnt3a; human gene: WNT3A)
Mouse genomic sequence (SEQ ID NO: 55)
Mouse mRNA sequence (SEQ ID NO: 56)
Mouse coding sequence (SEQ ID NO: 57)
Human genomic sequence (SEQ ID NO: 58)
Human mRNA sequence (SEQ ID NO: 59)
Human coding sequence (SEQ ID NO: 60)

TABLE 11

(mouse gene: Ly6e; human gene LY6E)
Mouse genomic sequence (SEQ ID NO: 61)
Mouse mRNA sequence (SEQ ID NO: 62)
Mouse coding sequence (SEQ ID NO: 63)
Human genomic sequence (SEQ ID NO: 64)
Human mRNA sequence (SEQ ID NO: 65)
Human coding sequence (SEQ ID NO: 66)

TABLE 12

(mouse gene: Rasa2; human gene RASA2)
Mouse genomic sequence (SEQ ID NO: 67)
Mouse mRNA sequence (SEQ ID NO: 68)
Mouse coding sequence (SEQ ID NO: 69)
Human genomic sequence (SEQ ID NO: 70)
Human mRNA sequence (SEQ ID NO: 71)
Human coding sequence (SEQ ID NO: 72)

TABLE 13

(mouse gene: Gata1; human gene GATA1)
Mouse genomic sequence (SEQ ID NO: 73)
Mouse mRNA sequence (SEQ ID NO: 74)
Mouse coding sequence (SEQ ID NO: 75)
Human genomic sequence (SEQ ID NO: 76)
Human mRNA sequence (SEQ ID NO: 77)
Human coding sequence (SEQ ID NO: 78)

TABLE 14

(mouse gene: Fkbp5; human gene FKBP5)
Mouse genomic sequence (SEQ ID NO: 79)
Mouse mRNA sequence (SEQ ID NO: 80)
Mouse coding sequence (SEQ ID NO: 81)
Human genomic sequence (SEQ ID NO: 82)
Human mRNA sequence (SEQ ID NO: 83)
Human coding sequence (SEQ ID NO: 84)

TABLE 15

(mouse gene: Rel; human gene REL)
Mouse genomic sequence (SEQ ID NO: 85)
Mouse mRNA sequence (SEQ ID NO: 86)
Mouse coding sequence (SEQ ID NO: 87)
Human genomic sequence (SEQ ID NO: 88)
Human mRNA sequence (SEQ ID NO: 89)
Human coding sequence (SEQ ID NO: 90)

TABLE 16

(mouse gene: Icsbp; human gene ICSBP1)
Mouse genomic sequence (SEQ ID NO: 91)
Mouse mRNA sequence (SEQ ID NO: 92)
Mouse coding sequence (SEQ ID NO: 93)
Human genomic sequence (SEQ ID NO: 94)
Human mRNA sequence (SEQ ID NO: 95)
Human coding sequence (SEQ ID NO: 96)

TABLE 17

(mouse gene: Bmi1; human gene BMI1)
Mouse genomic sequence (SEQ ID NO: 97)
Mouse mRNA sequence (SEQ ID NO: 98)
Mouse coding sequence (SEQ ID NO: 99)
Human genomic sequence (SEQ ID NO: 100)
Human mRNA sequence (SEQ ID NO: 101)
Human coding sequence (SEQ ID NO: 102)

TABLE 18

(mouse gene: Runx1; human gene RUNX1)
Mouse genomic sequence (SEQ ID NO: 103)
Mouse mRNA sequence (SEQ ID NO: 104)
Mouse coding sequence (SEQ ID NO: 105)
Human genomic sequence (SEQ ID NO: 106)
Human mRNA sequence (SEQ ID NO: 107)
Human coding sequence (SEQ ID NO: 108)

TABLE 19

(mouse gene: Il2ra; human gene IL2RA)
Mouse genomic sequence (SEQ ID NO: 109)
Mouse mRNA sequence (SEQ ID NO: 110)
Mouse coding sequence (SEQ ID NO: 111)
Human genomic sequence (SEQ ID NO: 112)
Human mRNA sequence (SEQ ID NO: 113)
Human coding sequence (SEQ ID NO: 114)

TABLE 20

(mouse gene: Nfkb1; human gene NFKB1)
Mouse genomic sequence (SEQ ID NO: 115)
Mouse mRNA sequence (SEQ ID NO: 116)
Mouse coding sequence (SEQ ID NO: 117)
Human genomic sequence (SEQ ID NO: 118)
Human mRNA sequence (SEQ ID NO: 119)
Human coding sequence (SEQ ID NO: 120)

TABLE 21

(mouse gene: Fyn; human gene FYN)
Mouse genomic sequence (SEQ ID NO: 121)
Mouse mRNA sequence (SEQ ID NO: 122)
Mouse coding sequence (SEQ ID NO: 123)
Human genomic sequence (SEQ ID NO: 124)
Human mRNA sequence (SEQ ID NO: 125)
Human coding sequence (SEQ ID NO: 126)

TABLE 22

(mouse gene: Nfkbil1; human gene NFKBIL1)
Mouse genomic sequence (SEQ ID NO: 127)
Mouse mRNA sequence (SEQ ID NO: 128)
Mouse coding sequence (SEQ ID NO: 129)
Human genomic sequence (SEQ ID NO: 130)
Human mRNA sequence (SEQ ID NO: 131)
Human coding sequence (SEQ ID NO: 132)

TABLE 23

(mouse gene: Flt3; human gene FLT3)
Mouse genomic sequence (SEQ ID NO: 133)
Mouse mRNA sequence (SEQ ID NO: 134)
Mouse coding sequence (SEQ ID NO: 135)
Human genomic sequence (SEQ ID NO: 136)
Human mRNA sequence (SEQ ID NO: 137)
Human coding sequence (SEQ ID NO: 138)

TABLE 24

(mouse gene: Dntt; human gene DNTT)
Mouse genomic sequence (SEQ ID NO: 139)
Mouse mRNA sequence (SEQ ID NO: 140)
Mouse coding sequence (SEQ ID NO: 141)
Human genomic sequence (SEQ ID NO: 142)
Human mRNA sequence (SEQ ID NO: 143)
Human coding sequence (SEQ ID NO: 144)

TABLE 25

(mouse gene: Znfn1a1; human gene ZNFN1A1)
Mouse genomic sequence (SEQ ID NO: 145)
Mouse mRNA sequence (SEQ ID NO: 146)
Mouse coding sequence (SEQ ID NO: 147)
Human genomic sequence (SEQ ID NO: 148)
Human mRNA sequence (SEQ ID NO: 149)
Human coding sequence (SEQ ID NO: 150)

TABLE 26

(mouse gene: Tbx21; human gene TBX21)
Mouse genomic sequence (SEQ ID NO: 151)
Mouse mRNA sequence (SEQ ID NO: 152)
Mouse coding sequence (SEQ ID NO: 153)
Human genomic sequence (SEQ ID NO: 154)
Human mRNA sequence (SEQ ID NO: 155)
Human coding sequence (SEQ ID NO: 156)

TABLE 27

(mouse gene: Stat5b; human gene STAT5B)
Mouse genomic sequence (SEQ ID NO: 157)
Mouse mRNA sequence (SEQ ID NO: 158)
Mouse coding sequence (SEQ ID NO: 159)
Human genomic sequence (SEQ ID NO: 160)
Human mRNA sequence (SEQ ID NO: 161)
Human coding sequence (SEQ ID NO: 162)

TABLE 28

(mouse gene: Sema4d; human gene SEMA4D)
Mouse genomic sequence (SEQ ID NO: 163)
Mouse mRNA sequence (SEQ ID NO: 164)
Mouse coding sequence (SEQ ID NO: 165)
Human genomic sequence (SEQ ID NO: 166)
Human mRNA sequence (SEQ ID NO: 167)
Human coding sequence (SEQ ID NO: 168)

TABLE 29

(mouse gene: Mdm2; human gene MDM2)
Mouse genomic sequence (SEQ ID NO: 169)
Mouse mRNA sequence (SEQ ID NO: 170)
Mouse coding sequence (SEQ ID NO: 171)
Human genomic sequence (SEQ ID NO: 172)
Human mRNA sequence (SEQ ID NO: 173)
Human coding sequence (SEQ ID NO: 174)

TABLE 30
(mouse gene: Prlr; human gene PRLR)

| | |
|---|---|
| Mouse genomic sequence | (SEQ ID NO: 175) |
| Mouse mRNA sequence | (SEQ ID NO: 176) |
| Mouse coding sequence | (SEQ ID NO: 177) |
| Human genomic sequence | (SEQ ID NO: 178) |
| Human mRNA sequence | (SEQ ID NO: 179) |
| Human coding sequence | (SEQ ID NO: 180) |

TABLE 31
(mouse gene: Top1; human gene TOP1)

| | |
|---|---|
| Mouse genomic sequence | (SEQ ID NO: 181) |
| Mouse mRNA sequence | (SEQ ID NO: 182) |
| Mouse coding sequence | (SEQ ID NO: 183) |
| Human genomic sequence | (SEQ ID NO: 184) |
| Human mRNA sequence | (SEQ ID NO: 185) |
| Human coding sequence | (SEQ ID NO: 186) |

TABLE 32
(mouse gene: Dusp10; human gene DUSP10)

| | |
|---|---|
| Mouse genomic sequence | (SEQ ID NO: 187) |
| Mouse mRNA sequence | (SEQ ID NO: 188) |
| Mouse coding sequence | (SEQ ID NO: 189) |
| Human genomic sequence | (SEQ ID NO: 190) |
| Human mRNA sequence | (SEQ ID NO: 191) |
| Human coding sequence | (SEQ ID NO: 192) |

TABLE 33
(mouse gene: Fli1; human gene FLI1)

| | |
|---|---|
| Mouse genomic sequence | (SEQ ID NO: 193) |
| Mouse mRNA sequence | (SEQ ID NO: 194) |
| Mouse coding sequence | (SEQ ID NO: 195) |
| Human genomic sequence | (SEQ ID NO: 196) |
| Human mRNA sequence | (SEQ ID NO: 197) |
| Human coding sequence | (SEQ ID NO: 198) |

TABLE 34
(mouse gene: Tk2; human gene TK2)

| | |
|---|---|
| Mouse genomic sequence | (SEQ ID NO: 199) |
| Mouse mRNA sequence | (SEQ ID NO: 200) |
| Mouse coding sequence | (SEQ ID NO: 201) |
| Human genomic sequence | (SEQ ID NO: 202) |
| Human mRNA sequence | (SEQ ID NO: 203) |
| Human coding sequence | (SEQ ID NO: 204) |

TABLE 35
(mouse gene: Nupr1)

| | |
|---|---|
| Mouse genomic sequence | (SEQ ID NO: 205) |
| Mouse mRNA sequence | (SEQ ID NO: 206) |
| Mouse coding sequence | (SEQ ID NO: 207) |

TABLE 36
(mouse gene: Zfhx1b; human gene ZFHX1B)

| | |
|---|---|
| Mouse genomic sequence | (SEQ ID NO: 208) |
| Mouse mRNA sequence | (SEQ ID NO: 209) |
| Mouse coding sequence | (SEQ ID NO: 210) |
| Human genomic sequence | (SEQ ID NO: 211) |
| Human mRNA sequence | (SEQ ID NO: 212) |
| Human coding sequence | (SEQ ID NO: 213) |

TABLE 37
(mouse gene: Vdac1; human gene VDAC1)

| | |
|---|---|
| Mouse genomic sequence | (SEQ ID NO: 214) |
| Mouse mRNA sequence | (SEQ ID NO: 215) |
| Mouse coding sequence | (SEQ ID NO: 216) |
| Human genomic sequence | (SEQ ID NO: 217) |
| Human mRNA sequence | (SEQ ID NO: 218) |
| Human coding sequence | (SEQ ID NO: 219) |

TABLE 38
(mouse gene: Nfatc1; human gene NFATC1)

| | |
|---|---|
| Mouse genomic sequence | (SEQ ID NO: 220) |
| Mouse mRNA sequence | (SEQ ID NO: 221) |
| Mouse coding sequence | (SEQ ID NO: 222) |
| Human genomic sequence | (SEQ ID NO: 223) |
| Human mRNA sequence | (SEQ ID NO: 224) |
| Human coding sequence | (SEQ ID NO: 225) |

TABLE 39
(mouse gene: Syk; human gene SYK)

| | |
|---|---|
| Mouse genomic sequence | (SEQ ID NO: 226) |
| Mouse mRNA sequence | (SEQ ID NO: 227) |
| Mouse coding sequence | (SEQ ID NO: 228) |
| Human genomic sequence | (SEQ ID NO: 229) |
| Human mRNA sequence | (SEQ ID NO: 230) |
| Human coding sequence | (SEQ ID NO: 231) |

TABLE 40

(mouse gene: Gnb1; human gene GNB1)
Mouse genomic sequence (SEQ ID NO: 232)
Mouse mRNA sequence (SEQ ID NO: 232)
Mouse coding sequence (SEQ ID NO: 234)
Human genomic sequence (SEQ ID NO: 235)
Human mRNA sequence (SEQ ID NO: 236)
Human coding sequence (SEQ ID NO: 237).

TABLE 1

MOUSE NOMENCLATURE
ICSGNM Nyc
Celera mCG1625
HUMAN NOMENCLATURE
HGNC MYC
Calera hCG1791915
MOUSE SEQUENCE—GENOMIC
CTTCCTTCTCCCTTCACTGAAACTGAACTGACCTTGGGATGGCGAAACCCCCACTTCATGGCACCCACTCTAAAAGATGGCTGTTGGTCAAACA
GCCTAGCAAGCTCTAAACCAGGTACTCTGCACCTGCCCTCTGACGCCCTGGGATTATATCGCAGACCGTAAGAACAGGAATGACATAATTAAAA
CCAGCCTCAGAAGCTGCTGAAGGCATTAGAACTAAAGCCCAGAAAAGCAACAAACTGTTACCATCAAAGCGTGAAAGAGGAAACGGTTAATCTT
TTCAAGGGAAGTGAAGCAACTCGGAAGGCCATAGGAATCCACATCACCTGAACTGCAGACCAAGCCTTGGAACTGGGGCTTTCAAGCTACATCT
GCTACTCACACCTTTGACCTCTCTGGGTGAATTGTTTTCTTACTAGTCACCAATGTTTTCAAACCTGGATTTTCACACTCCCTCGAATGCCTGG
AAGGGCTGCTACCAGCGGACCTATATTGCAGGGCCACCAACTGCCAGGTTCTGAGCCACAGCTCTTAGCTAGGACCAAGCAAATGGTCCCCAGT
GTGTTACAGTCTCCTGTCCTCCAAAGTGAGTTCCTCAGATGCACTCCTTTCCAATCCTNNNNNNNNNNNNNNNNNNNNGATTTTCCTGTGGGCA
CATCAGCTTCCTATGCTGAAAAAAACAGAGAGTAGTAGAAAGGGAAAGCCTAATCAAGCCTGCAGCTGAGAGATTGCCCTGTGCCACAACCCCC
AAGTCCACCCCCTTATAGGACACCACCTTAGTCATTCAGGGAAAGCCTTAATGGATTGCCTTGATTCGCCTTGATTCCATAATCTAATTGACCA
TCAGTAGAGCAAGCTGCACCAACTCCTATCGTAAATGAAGACAGGGGTGAGACTGGAAGCGTCAGATACTGGAAAATCTGAGATTCTCTCCCTT
GGTTTCTCATCTTCATCAGTTTCAACCAAAAAAGAAGGAAGCTTTAACCACGACTCCTTGTTTTTGTCAAAAGAATATGCTAAAGTACACATTC
CCATTATTGCTCATGAGACCTTGTTCATATGTCACCAAGAATATTCACTGGTATATGTTTGTTCATACCTTCTTAAACATAATTTAAGATTTCT
TTATTTGATGTATGTGTTTTGCTTGCATAAGGTATATGTACCACGTGTGTGCTTGTACCTGCACAGGTCAGGACAAGGTTTCAGTTTCCCTGGA
TTGATGACTGTGAACTATCATGTGTATGCTGTGAACTGAACTAAGGTCCTCTGTGGAAGCAACAACTGTTCTTAACTGATGATCCATCTCTCCA
GCTTCCGCTCAGAAATTTTCACACTTAAAGAAATAAGGGCTGGCTGGTGAGATGGCTCAGCGCTTAAGAGCACCGACTGCTCTTCCAAACGTCC
TGCGTTCAAATCCCAGCAACCACAAAGAAATAAGGGCAGGGGCTAGAGAGATGGCTCAGCAGTTAAGAGCACTGACTCCTCTTCCAGAGGTCCT
GAGCTCAATTCCCAGCAACCACATGGTGGTTCACAACCATCTCTAATGTGATCTGATGTCCTTTTCTGATATGTCTGAAGACAGCTAAATTTTA
CTCATATACATAAAATAAAGAATTCTTTAGAAAGAGAGAAAGGAAGGAGGCAAGGGAGGAAGGAAGGGAGAGAGAGAGAGAAAGAACGGAAGGG
AGGGAGGGAGGGAGGGAGGGAGGGAGGNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NCCCTCCTCCTAGCTTTCTGAAGCCAGTCTTCTCCGGTTTGCCTTCAGAACAAGATGTAAAACTCTCAGCTCCTTTAGCTCCATGCCCGGACTG
GATGCTGCCACACTTCCTGCCTTGATGATAATGAACTGAACCTCTGAGCCTGTAATCCAGCCCCAATTAAACATTGTCCTTTATAAGAGTTGCC
TTGCTCATGATGTCTCCTCACAGCAATGGAAACCCTAACTAAGACAGCAAGTTTAGCAGTAAATGGGGTGGCAGAGACAGGGTGATAGCAGGAA
ATACTTTGACATAGCTAGTGAGACAGCAGGTAATGACAGTAGCCAAACCAGCCTGATAAACAAACAGCTAGCCCAGAATGCATAGCACAGCAGC
AAAAACATTGGAGAACTGGCTCAATGACAAAGTGGAAGCAGGAAAGGCAATTCCTAAATGTTGTCATCTGATCACCATATCTGCACACTGAAGC
ACACACACACACAAATAAACAACACTTTAAAGAAATAAATATTACATGATGATTGCAACAACCACAGGCCATCGGAAAAGAAAGAGGAAAAAAA
ATCAATGGAGCAAAGAGACCAGGCTGCACACACAGGAGCAACATCAGGAGACCCAAATTCACCCAAATGTTCTCAATGTCAGCATGTTTGCATC
TAGGTCCCAACACTCTAAGGGGATTGGCAGGATCCCTGACCCCGACTCACTAAATACCAATAGGAAGCCCCAACCCCCACCACCTATAACAACC
TCAAATATCTCCTCCTGTTGGTCAATGTCCCGTGGAAGCAACATTACCTCTAGTTGAGAGGTACTGATCTCTAGAATCCAAAAGTGTGAGCATT
TCCAGAAGCTTTCCCAGCAAGCAACAAAAAAGACATGGTCTCACGGTCACCGGTTGTGGGGAAGATAGCTTGAGTGCCTTTAGGGAGGTTCATT
CAATGATAAACTCCACATACAGTAGTCAGGAGGGAAATGGAGAACGTGAAACTGAGAGCAAGCAAGGCTGAGCTGCACTCAAAATTCTACCTAC
AGAAGTCAAGAGCTGTGACCCATAGTTTGGGGTCTGTTCTAGCCAATAAGAGGCACAGGCTACTATTGCACTCGCTTCTAAATGAAAGTCAAAG
ATTACGGACAGAAAAAAAGGAAGAAAAAAAAGTAAAAAAGAAAGCAAGACCCGTCATGCACCTCCTCCCCAGTCACCTTTACCCCGACTCAGCA
AGGGTCCATCATGGAGACAGATGCCGCCCAAAAGGACCATCACAAAGGCCGGCCAGTGAACAAAAGTGCAAGATTGACTTGGGTTTGCCATTTA
CAGAACAATAAACGAAGCCTTTGAAGCACTCTTAAACAAAAGAGCCATTTGTCTATGCTGGTGGCTACAAAGGAGAACAGGATTGCAGCAGAGG
ACTAAGAAAAGAAAGAGAAAATGCCAAACTGAGATGTGCCTGGACTTTACCGGGTCCTGCCCAGAGACCTTATATGCCAGGACCTAAGATCTAA
AAGCCACTGGCTAAGAAAACAATCAAATATGCTTAATGCAATACAGGCAATGTAGGTTCTTGGCTATTGTGTTAAAAATATCTACTAATTTTTT
TTCCTTGTCAGCTGCTTTAATTTATGCAGTATGGGAAGGGATCACATCGGAAAACACAAGGGAAGAACAGAAAAGAAACACCGTACATTTTCAA
ATGCACAAAGATCTCATGCACTGCCTTCTGTTTCTGTGCCTATGAATTAGTGATGCTAAATTTTATCTTATTTTCTTCCCTGGTGGGGGGGGGG
GGGACGGCGGGGAGGGTGACTGTGTTCCTCTCTGTGGTGGCATAGCTGCCATTTACATGCCTGTGAGGTCCTCTTTTCCTTTAGCATGCCATGG
CTAGCTTGGTTTTTAATCTAACAGCGCTTCTTTCCAAATGATGTTTCTGCGGCAGACAGCTATTTATCATTGTCTTTGCTCTCGTCACTTCCCG
ACAGGACTCGTGAGAACTGAAGTAATTTTGAAATCGTAGCCCGTCCTTAGGGTCTCCTGCTTCTCTCCCGAGTAGGAGCTAGGTTTTTGATCCA
GTTTGTGTGGATTTAGTGACTGTTTTTATACATGGAAGGTTAGCTCAGTGGTGATGGTTGGGGGGGGGGGCTCTTTGAAGCATGCAGAAAAGAG
ATTCAAGAACATTGGGGGATGTTATCTAGTGGTTTTCTCCTTGTTGTTACAAGAGTGACTTTGGAGAAGAGGGGTATACTTTGGCTCAGAGTTT
GACGGAAAAATCTGGGATGGAAGAAGTCATTGTGGGAGCAGCTGGAAGCTTTGTCACTTCGGGAGCCTTGTCATACTCTAGCCAAAGTTAGCAA
GCAGGTTGGAGGGTGGGGAGACAGATTCTGGAGTTTGGCGCTTCTGTTTTATACCTTCTCTACCCACATTGTGAATAGACCTGACCCCTGGCTT
GACACAGATCACATTACTGTGAGTAGGTCTTCCTTCTACACATATACCTCTCTAGAAATACCCTCATAGGTAACACCAGATCTGCATCTCCTAA
GTGATCAAGTCGACAATCAGAACTAACCATCATAGGCAGGGCTGCAACACACCTTGAATCCCGTGACTTAAGAGGCAAACACAGGTGGCTCTCT
GAGTTCAAGGCCTGCCTGGTCTACAGAGTTTCAGGACAGTCAGGGAAACCCTACCTTGAAAAACAAAAAACAAAAAACAAAAAACAAAAAAAAA
GAAAAGAAAAGAAACACCCAAATTAAGCGTCTCAAGGATGACCGTTCTTGGTATGTTATTCACACCACATATTTGGACCTCCCCTCTTGTGTCT
TTGCAAACTAAGATCTTCGGCCGACTTGTTCATTCTAGCTGGGAATATACAGTTCAGTGACTGAATGAATCCTGACCACAAATGACGCCCTGTT
TCAATACTGAGCACACCTTCCTCTCCTTCCCATAACAGCCCTTTCTTTCTGGAGGTCTTCCTTTTTTTCTTAAAAATGTAAGAGCATCATGACT
GTTAGTATGAGACAGTGAGGTTCTGGGCACTGTGATTCTTAGTTTTTCATACTGTAAGAGGGAATGTACTCTCTGCCATCGGGACACCCAGTGG
AACTGCTCACCTGGAGTCTTGCCTCCACGAAGACTAGGATCTTCTGGAAAGCTGTGTGCTTTGGACATCTAACTCACATCCAGCAATAGCTCCC
AGAAAAGGACCTCAGAGTTACAACGTTCCAGAGGAACAAAACAGTGGGACATCTTCCACCATCTTCCAAAAGGAAACCTCAGAACAGAACAAGC
AACCTTTGTCCTTAGTAACCTTGTCCACAGACGCACGCATCGCTGTCATCCCAACACTGGCAAGTCCAACGCAAGCGAACAGCAAGTTCAAAAC
TAGCTTCAACTCGCTCCATAGTGAGCCCCTATCTCAAAACATCACCCGCTAACTATGTAGCTCAGAGACAAAGCCCTTGTCCAGTGTTTGAAAC
ACTCTCGATTTGATTCCCAACAATAAAAACAACAACATACACTCAGATACTCACACACACACACACACACACACACACACACACACACCCAACT
CCTTAAACACTAAAATTTAGTGTACGTAGAGTGGCACAGGAAACTCTAATTCCAGCACTACAAACCCTGAACCAGTTTTTATTTTAGTTAGAAG
CCATCCTAGGCTACCTTCTCAACAAGAACATTCCAATCACTACACTTTATTGACAAGATTATAAACTTGTGGCTTTCCTGTCCTTTTTAAAGTA
TTTTTTAAAATTTATTTTACAATGAAGTGGTACCCTTTCATTTCCTAATATGTAAAATGGGGATGTCCCCTACCTTCCTGTTACTTAAAAGCAA
CACCCCGGGTGATGTCATCAGGCTGGGGTACAGTACGGGCAAGTCCCCAAACCACAGGCTATTCCGCTACCTGGGTTCCCACACCTACACTGGA
CCGGCATGATTTGGACGTAAGATCTTAAAATAATCCATAATTACACCTTTTCAAAAAGGACCTTTTGAGGGTCAGCTCTACCCACTGTCATATC
TGTTGGTGCTGTCCTCTGTTTAGCGAGAGGCAGACACAAAGCGGGTGTGCGCAACCTCTAAGAGAGTAATAGTAAAGAGACAAGCCTAGGAAAT
CTTCCCCAAGAGGCCTTTTGGGGAAGGCTATCCCCCCTTCCGTGGATGTTGAATACCTTGCCTGTTGCTCAGATGCAGTCAAGCATCTGATGAT
GCTTGAGGCATGATGCTGAGACCACTGTGTAGGTTGGTACAAATTCCCTTTCTATTATACCCACCCTTATGCAGTTATTTGGGTTTAAATTTTC
AACGTTTTAAAAAAAAAAAAAAAAGTATTGCCGGGCAATAGTGACGCAGGCCTTTAATCCCAGCACTCGAAAGACAGAGGGCAGCCACATCTCT
GAGTTTGAGACCAACCTGGATCGTCTAACGCCTTGA&CTCAAGAGATCTATCCACCAGCTTGTATCCCATTTAATTGAGTGATTTGTTTGCTCT
CTCTCTCTCCCGCTCCCCCTCTCTCCCCCCTCTCCCTCCCCTTTTTCAGTTCTTTATTTATATTTGAATATTATCTCTCTCTCAGATGTGTACT
TGGTGACGGTCTTTTCTACAAAGGATTATCTCTTTCATGACACGCCATTTATTAATCTGCTTGTCCCTCGACCTTCTCTACAGTAGCGTCACCT
GATCCTCTAGCTTCTCCATCCCAGATGATTGGGATTCAAGGCATTCATCACCACGCCTACCTAAAATTCCAACCTTTTAATAAAATGGCTCTCT
AAAATATTTTGGCAAAGGAATCTCACTCTTTCTCTTCACTAATATAGTTCAACCACCCAATTCGTAAGCAGTCGGGCTGAGGGTGAGCATCCTT
AAGAACATAAAAGCAAAATTTGGAGGCTCAAGATTCAGTTTAGTTGCTAGAGGGCTCACATAGCATGCCCTCCCCACCCGGGATTCCATTCTCA
TTTATCGAGGCATAAGGCCAGGTGTGGTGGCATATGTGCTGGCATCCATAAGATCTTTTATAAACAAGAGGAAGAGGAAAAACAGTTATACAAA
ACTAATAAAACTGTGTGAGTTTCAGGCTAGCAAAGAATACTCCTGAGAATCTCTCTCAAAAAAAAAACCCCAAAATAATAATAATGATAATAAT
GATAATAATAATAATAATAATAAAACAAGGCAATAATAAGCTAATGCTCCTGCCTTGCATTCTGACTCCTTTTGCCCAGTAAAATTCAATGCTC
TGCTTTGACACGTCCAGCTTACAACACGACAAAGGTGTGAGACATCGGTGCTAAAACTTGTTCTATTGCCTTTCCGTTTCTGTTAGATCTCCTT
CACTTGTATACCTGTGACTCATTCATTTTCCCCATCCACAACTAGGGCTCTCTTTTGGGTGACTCTCAGAAAGTAGGCATCGGTTCATCTACTC
CCCCTCTTTGCAACTGAATAGCCACCTCTGAACCATTTTTTTCTCTAGTAATTTCTCTTCCTTTGCCTCCTTTGCAGCCTAAAAGAGTCATTTA
AAGGATGACCGGAAGCTTGTCTTAGGCAAGGAAGCATCTTCCCAGAACCTGCAAACCCTCCAGCCCTGCCCCCATCCCACCTCCGCCCTCGTTG
GCTTCGCAACGCTGTGGTCTCTGTGGCCAGTAGAGGGCACACTTACTTTACTTTCACAAATCCGAGAGCCACAACCCGGGTGGTGGGGGGTGAG
GGGGCGGGGAAAGAGTCTCTGCAGCAAAACGCAGACTAGGGATTGGTGGCTCTTGGTGTTTGAGGCAAAATCCTAGAGGCTGTAGTCATTTTCC
AATCCTTAAAGCTGAATTGTGCAATGAGCTCGATGAAGGAAGATACTATCATTCAACAGCTGAATCCTAAATTGCAAACTCAGTGGCTAATAAC
AACTTTGAACAATGAGCACCTTATACACGCTACTGTATTTTCTTTTCTTTCTTTTTTTTTTTTTTTTTTTTTTTTAAACCGGGTAGCAGTGAGA
GAGGTTTCTTTAAGTGCCTTGGGGCGACGAGTCCGGAATAAGAAGACTTCTTTGGGTTTTAAAGTGTAGGATAAGCAAATCCCGAGGGAATATC
CATTATATAATAAATCTAGAACCAATGCACAGAGCAAAAGACTCATGTTTCTGGTTGGTTAATAAGCTAGATTATCGTGTATATATAAAGTGTG
TATGTATACGTTTGGGGATTGTACAGAATGCACAGCGTAGTATTCAGGAAAAAGGAAACTGGGAAATTAATGTATAAATTAAAATCAGCTTTTA
ATTAGCTTAACACACACATACGAAGGCAAAAATGTAACGTTACTTTGATCTGATCAGGGCCGACTTTTTTTTTTAACTGCATAATTACGATTCC
AGTAATAAAAGGGGAAAGCTTGGGTTTGTCCTGGGAGGAAGGGGTTAACGGTTTTCTTTATTCTAGGGTCTCTGCAGGCTCCCCAGATCTGGGT
TGGCAATTCACTCCTCCCCCTTTCTGGGAAGTCCGGGTTTTCCCCAACCCCCCAATTCATGGCATATTCTCGCGTCTAGCGCCTTGATTTTCCC
CACCCCAGCTCCTAAACCAGAGTCTGCTGCAAACTGGCTCCACACGGGCAAAGAGGATTTGCCTCTTGTGAAAACCGACTGTGGCCCTCGAACT
GTGTGGAGGTGTATGGGGGTGTAGACCGGCAGATACTCCTCCCGGAGGAGCCGGGTAGAGCGCACCCGCCGCCACTTTACTGGACTGCGCAGGG
ACACCTACAGGGGAAACAGCCGCCTCCACACCACCCGCCGGGTGGAAGTCCGAACCGGAGGTGCTGGAGTGTNNNNNNNNNNNNNNNNNNNNTG
ACGCGGTCCAGGGTACATGGCGTATTGTGTGGAGCGAGGCACCTCTTCCACCTGCGGTGACTGATATACGCAGGGCAAGAACACAGTTCAGCCG
AGCGCTGCGCCCGAACAACCGTACAGAAAGGGAAAGGACTAGCGCGCGAGCAAGAGAAAATGGTCGGGCGCGCAGTTAATTCATGCTGCGCTAT
TACTGTTTACACCCCGCAGCCGGAGTACTGGGCTGCGGCGCTGACGCTCCTCCTCCTCTTTCCCCGGCTCCCCACTAGCCCCCCTCCCGAGTTC
CCAAAGCAGACGGCGGCCAAGCGACACCAGCAAAAAAAAATAGAGAGAGGTGGGGAAGGGAGAAAGAGAGATTCTCTGGCTAATCCCCGCCCAC
CCGCCCTTTATATTCCGGGGGTCTGCGCGGCCGAGGACCCCTGGGCTGCGCTGCTCTCAGCTGCCGGGTCCGACTCGCCTCACTCAGCTCCCCT
CCTGCCTCCTGAAGGGCAGGGCTTCGCCGACGCTTCGCGGGAAAAAGAAGGGAGGGGAGGGATCCTGAGTCGCAGTATAAAAGAAGCTTTTCGC
GCGTTTTTTTCTGACTCGCTGTAGTAATTCCAGCGAGAGACAGAGGGAGTGAGCGGACGGTTGGAAGAGCCGTGTGTGCAGAGCCGCGCTCCGG
GCCGACCTAAGAACCCAGCTCTGCAGTCAGAGCGGCTTTCCCTCCCAGCCTGCCGCCCACTCTCCCCAACCCTGCGACTGACCCAACATCAGCG
GCCGCAACCCTCGCCGCCGCTGGCAAACTTTGCCCATTGCACCGCGCAGACACTTCTCACTCGAACTTACAATCTGCGACCCACGACACGACTC
CCCAGGCTCCGGGGAGGGAATTTTTGTCTATTTGGGGACAGTGTTCTCTGCCTCTGCCCGCGATCAGCTCTCCTGAAAAGAGCTCCTCGAGCTG
TTTGAAGGCTGGATTTCCTTTGGGCGTTGGAAACCCCGGTAAGCACAGATCTGGTGGTCTTTCCCTGTGTTCTTTCTGCGTCTTGAATGTAGCG
GCCGGTTAGGACAGTCTTTCTTCCATTCCTGTGCTTTTGACACTTTTCTCAAGAGTAGTTGGGCTACGCTCGGGTACATCTCAGTCGGGGTAGA
GCGACTTGTCAAGATGACAGAGGAAAGGGGAAGGGAAAAACCGGGATGCATTTTGAAGCGGGGTTCCCGAGGTTACTATGGGCTGACGCTGACC
CGGCCGGTTGGACATTCTTGCTTTGCTACATTAATTGATATGTGTCCTTTGAGGGGTCAAACCGGGAGGTCGCTTCGTGGTGGCCAAAGAAAGC
CCTTGGAATCCTGAGGTCTTTGGAGAAGGGATTACCTTTTGCGTTTGGGAGCGAGAAGGCTCCGTAGCTTCTGACTTACCAGTCTCTGAGAGGC
CATTTAAATTTCAGCTTGGTGCATTTCTGACAGCCTGGGACCGACACGGAGGTGCGTCCCGCCCGCCAATCCCCGGCGGCGATCGCAACCCGTC
CCTGAGCCTTTTAAGAAGTTGCTATTTTGGCTTTAAAAATAGTGATCGTAGTAAAATTTAAGCCTGACCCCCGCGGCACTAGGACTTGATGTTG
GGCTAGCGCAGTGAGGAGAAGCAAAATTGGGACAGGGATGTGACCGATTCGTTGACTTGCGGGAAACCAGAGGGAATCCTCACATTCCTACTTG
GGATCCGCGGGTATCCCTCGCGCCCCTGAATTCCTAGGAAGACTGCGCTGAGTCGTGATCTGACCGGTTCCGTAACAGCTGCTACCCTCGCCGC
GGAGAGGGAAGACGCCCTGCACCCAGTGCTGAATCGCTGCACGGTCTCTGGTGCAGTGGCGTCGCGGTTTAGAGTGTAGAAGGGAGGTGTCTCT
TATTATTTGACACCCCCTCCCCTTTTATTTCGACACGCTTGTCATAGCCGGAGACTGAGCTCTCTCCTCCAAGTCAGCAATCGGAAACAAAAGC
CGGCAAAGGAAGGAAGGGGGCGCGCTGGGGGTGGAGAAAGAGGAGGGCGGAGAGGGGCGCCGCCGCCGGCTCCGTAGGAGCGCGGCGACGCCGC
GAATAGGGACTCGGACCCGGTCGGCGCCGCAGAGACCCGGCACACGGGAGGGGGCCGAGCGACGCGGCGCCTCTCGCCTTTCTCCTTCAGGTGG
CGCAAAACTTTGCGCCTCGGCTCTTAGCAGACTGTATTCCCTACAGTCGCCTCCCTCAGCCTCTGAAGCCAAGGCCGATGGCGATTCCTGGGCG
TCTGCACGGCTAAGTCCCTGCTCGAAGGAGGCGGGGACTCGGAGCAGCTGCTAGTCCGACGACCGTCACTGATAGTAGGGAGTAAAAGAGTGCA
TGCCTCCCCCCCAACCACACACACACACACACACACACACACACACACACACACACACACACTTGGAAGTACAGCACGCTGAAAGCGGAGTGGT
TCAGGATTGGGGTACGCGCTGCCCCAGGTTTCCGCACCAACCAGAGCTGGATAACTCTAGACTTGCTTCCCTTGCTGTGCCCCCTCCAGCAGAC
AGCCACCACGATGCCCCTCAACGTGAACTTCACCAACAGGAACTATCACCTCGACTACGACTCCGTACAGCCCTATTTCATCTGCGACGAGGAA
GAGAATTTCTATCACCAGCAACAGCAGAGCGAGCTGCAGCCGCCCGCGCCCAGTGACGATATCTGCAAGAAATTCGAGCTGCTTCCCACCCCGC
CCCTGTCCCCGAGCCGCCGCTCCGGGCTCTGCTCTCCATCCTATGTTGCGGTCGCTACGTCCTTCTCCCCAAGGGAAGACGATGACGGCGGCGG
TGGCAACTTCTCCACCGCCGATCAGCTGGAGATGATGACCGAGTTACTTGGAGGAGACATGGTGAACCAGAGCTTCATCTGCGATCCTGACGAC
GAGACCTTCATCAAGAACATCATCATCCAGGACTGTATGTGGAGCGGTTTCTCAGCCCCTCCCAAGCTGGTCTCGGAGAAGCTGCCCTCCTACC
AGGCTGCGCGCAAAGACAGCACCAGCCTGAGCCCCGCCCGCGGGCACAGCGTCTGCTCCACCTCCAGCCTGTACCTGCAGGACCTCACCGCCGC
CGCGTCCGAGTGCATTGACCCCTCAGTGGTCTTTCCCTACCCGCTCAACGACAGCAGCTCGCCCAAATCCTGTAGCTCGTCCGATTCCACGGCC
TTCTCTCCTTCCTCGGACTCGCTGCTGTCCTCCGAGTCCTCCCCACGGGCCAGCCCTGAGCCGCTAGTGCTGCATGAGGAGACACCGCCCACCA
CCAGCACCGACTCTGGTAAGCTACCCCATTCACAGCAGGGTAGGAAGCCAGAGGTTGGATGGACCTCCTTCTCCACCACTCATTGGCATTAATT
CAATTGGCCTCCGGGGCTCCCCCTTTCTTTCCCTTCTGTCTAAGAGCTCTTCATCCCTGGATTCCCGTGNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNTCACGAATTTCATTTGGGTTTTTAAATCTTCTCGCTTATCTTTCAGCTCCATC
CACTCCCTTTACCCCTCCTAAGCATTTTAATTACCCTGGGAAGGGTGTGAATGAGGATAAAGCAACTGATCTGGAGGGGGGTGAATTACCTGCT
TCTTTTCTTGACTGCCAGAAGAATATTTGAATTTAATGCATACGTTTGTCTAAGACCCAAGAGAACCAATGACAGAAGCTGGGACAGGCTTTAT
AGCCTTACAGCCAGGCACTAGTGAAAGTTCCTAAAGAATTGAAGAGCTGGGCTCTTTTGGGTGTTTTGTTTTGCTTTGTTTTTTCGTCCTCTTT
TGAACACTTCAAACCAATTCTGTTCAATTTGGACTTCTCCCCCCGTCCCAACACTCCCCCAACACCAGCACCTTTGGCAAAGCTGCAAGAACTT
TTTTTTTTTTTTTTTTTTTTTAATTGTGCTTCCAGTAAAATAGGGAGTTCCTAAAGTcATAGCAAGAGATTTGCAGCTATCGCTCACGGGAGCT
GAAAGGTTCTCGGTAAAGTCCCTTAAAAATAGGAGGTGCTTGGGAAATGTGCTTTGCTTTGGCTGTGTCTGAAGCCTCATTAAATCTTAGGTAA
GAATTGGCAAGGATACCATATCCTGGTACATGGTAATTTTCTCACCTCTCCCCTAACCCTGTTCTGCCTTTCTGGCAGAAGGCAACATGGTGTC
TGGATCTGATTCTTACTTTCTTCCCTTTCCAACTTGGTATTTGGATAGCATCGGTCAAATCCTATGTATAGCCTCCGGGATTCAGGAGGCGTGG
CTAACTGTGATCTTCCACTTCCTCCCTTACAGAAGAAGAGCAAGAACATCAGCAACAAATTGATGTGGTGTCTGTGGAGAAGAGGCAAACCCCT
GCCAAGAGGTCGGAGTCGGGCTCATCTCCATCCCGAGCCCACAGCAAACCTCCGCACAGCCCACTGGTCCTCAAGAGGTGCCACGTCTCCACTC
ACCAGCACAACTACGCCGCACCCCCCTCCACAAGGAAGGACTATCCAGCTGCCAAGAGGGCCAAGTTGGACAGTGGCAGGGTCCTGAAGCAGAT
CAGCAACAACCGCAAGTGCTCCAGCCCCAGGTCCTCAGACACGGAGGAAAACGACAAGAGGCGGACACACAACGTCTTCGAACGTCAGAGGAGG
AACGACCTCAAGCGCAGCTTTTTTGCCCTGCGTGACCAGATCCCTGAATTGGAAAACAACGAAAAGGCCCCCAACCTAGTGATCCTCAAAAAAG
CCACCGCCTACATCCTGTCCATTCAAGCAGACGACCACAAGCTCACCTCTGAAAAGGACTTATTCAGGAAACGACGAGAACAGTTGAAACACAA
ACTCGAACAGCTTCGAAACTCTGGTGCATAAACTGACCTAACTCGAGGAGGACCTCGAATCTCTCGTGAGAGTAAGGAGAACGGTTCCTTCTCA
CAGAACTGATGCCCTGGAATTAAAATGCATGCTCAAAGCCTAACCTCACAACCTTGGCTGGGGCTTTGGGACTGTAACCTTCAGCCATAATTTT
AACTGCCTCAAACTTAAATAGTATAAAACAACTTTTTTTTATGCTTCCCATCTTTTTTCTTTTTCCTTTTAACAGATTTGTATTTAATTGTTTT
TTTAAAAAAATCTTAAAATCTATCCAATTTTCCCATGTAAATAGGGCCTTGAAATGTAAATAACTTTAATAAAACGTTTATAACAGTTACAAAA
GATTTTAAGACATGTACCATAATTTTTTTTATTTAAAGACATTTTCATTTTTAAAGTTGATTTTTTTCTATTGTTTTTAGAAAAAAATAAAATA
ATTGGAAAAAATACAATTGGGCCAACTTGTGTTTTCTTTTTCCTCTTCCTCAAACTTCCTTTCCTCAATTACAGATTAAAAGAATTTGACCATT
TTCACAGGGTACGTTTACAAATATGGGAAGGGGTTATCATTGTTAAAATGGGGCTGGGGGTCCTCAGGATTTCTAAGTTGTCTACAGGATGCTT
TCTGTGGATAGTAATAAAAACCAGAGCTGTTAGTTAGGAATGGGCAAAAGGCAAGTGAGAAGGCTAGATGCAGGGAAGGGAAAAGCAAGAGGTT
AAAGATAACAGCTAAATATACAGGAGGAAGAGATGGCAGAATCTCCTACAGTTAACCGAAGCCATTCCCTGGTTCACCTCAACGCAAGGACTCT
GCCCTGCCAAAGAACTGGTGAGGGGAGGGAGAGAGAACCACCCTTTGTTCCTTGCCTCTTGCTCCCAGGTGATAGTCCCTTCACATCAGTATCT
CCTATGCTTCTGAAAAAAACAGAGGAACAGCATTACCACTGCTAAGTTGATCCTGCTTTTCCAAACAAGGACATACAAAGGTTCAGAGGGTTGC
AGAGCTTAATGGGTACCAACACAACAAAAATAACCCAGGGCCCCTGTCTTCAAAACAAGCCTTGCTGCCTTGCTTAGTTGGGTGTGCTTCCGCT
GGTTAGGGGTCTGAGAATGAGCGCTACAGGCTCCATTAGGAGCTTTGACAGAGTGCCACGAAAGAGGTACTATGATCTCTTTATACCTAGTACA
GGTAGAAACAGACATGAAATAACCCATCCATGCACCCAGGGGATTCAAAACCACTTTATCCTTAGTGCATCCAGGAGGAAGATCCTAGCTACGA
ACGACAGGGCAGGAGTCATTAAAGCTTCACTACTCAAGAACTGGTAGACTTCAGTTCCCCTCCTTCTTTGTGGATTGGGGGGTTGGGGTGGAGG
GTGTTGTGTGTACTCACAGGCACATAGCTCACTCCTACCTTACCCACTTACTTTTTAACATCAGATCCCTGCTTGCTCCTGGCAACAACCCAGT
TTAGAAGTGCTACTGGTCAGATCATAAAATAAAACCTTGTTTTACATGAGTCATTATTTTAGAAATTGCAAGCTCGCCTTCCTCCCAAGCACTT
TCACACACTCATGACACACGCTCATCAATGGCCCATGAGAAAGCCTTTTGGAACAGGTATCTTATTAATTATAAGCCTCTGAAAAGCCACAATC
CAAACCAGAAACTGAAACATGTACGTAAAGTCCAGGGAGAAAGGTACCCTAAATGTCCTAAATGACCCCATTGTCTCAGAGAAGGTCTCTACCA
TCCTGGAGTGTCTTTGGGACTTTAATTCACCATCATTCATAAACCAATAAGTAATTACTATCTTTGACACCCCCCCCCCATGATCTATTGAAGC
GTTTAACATTTAGTTTTTGATCTTATTTTTGGCTCTTTTAGACTCACCCTCAGCTTCAAACTGTTACACCCTCTATCCTACATTCAAAAGAAAA
CACAAACCCCAGTAGTAGTTTGAAATACTTTGAGTAGTTCAAAGGATTAGCAAAACGCCCGAGCACGGGGTGCTCTTAAATAGTTCGCTCTTTT
TCCCCTTTGTGACCCTAGGCTGGAGTGCAGCCCTGGGGTGACTCACTTCAGACCTGGGAAGGTGTTAGGTTGAATCACTCAGTCCAGGCAAGCC
CAAAGAATAGAGGAAAGCATTCCTCATTAGGAAAACAACTCCTGTTCCAAATGATCAGGAAACAAGTTTAGAGATTCAGATTTGGCTGTGGGGA
TGGAATCGAAGTATCACAGCTCCCTATCTGGGCACTTCTCAGCTTTACCAAGCCAGGGAATCGTCTGAAAACAGGACATCGGCCAGCTTCCTTC
CAGAAAGTCAGGCTGATCTTGACCATAACACAGGAAGGCTCTCCCAGCCAATCTGCAGTCCCAGGGGTCTGCAACATTGTGCAATCAAATTTAT
AGATAACAATCACGATGAGGGGTGAAGTGGGACGAGCTTTCAGCTTGGTCTGGGATATACAAGAATTACCTACTCTCTTCTGTTGTTCACCCAT
GACACTGGAACTCAGTTTCCCCGAGAGGATCCCTGGGATGGTGCTCTTCAGGATAAACTGAGTGAGGAGGAAGTGTGACTTTATCCTATCATTC
GGGGACAACACTAAAAAGCAATCAATTACACTTTAAGTTGAACAAAAGTTTCACAATACCAGGATAGTGCTTCAGTCATCCAAAGCTTGAACAC
GTATGTAGAGTCTTCTGCTGTTCTTTAAGATGGACTTTGGCACTAACCTTGGAAAAGAGGGCCAAAAAACCCACCCCCCAAAAACAAACAGAAC
AAAAAACGTGAGGGGATTTTCTCTGGTTTGTTTACAATAAGAGATGAGTCACAATAATGATTTTTTTTTTTTTTTTTAGCATGACTGCTAAGGA
ACCCTGAAGCATTTCTTCCCAACAAAAACTAATCTTTAGGTTATAACATGCTGGAAGATGAGCTTCAAGCCTGTCTATAAATACATCACTGGGT
ATCTTCAGGGAACAAACTCTTCACTTAAGGTTAGAGACCATCCTGGATCGATTTCAAGAACAGAGGTTTATTTTAAACAATCAACCCTGCGCTA
GAGATGTCGTTCAGTGATAAGACACTTGTCTAGCATGTATAAGGACCCATGCTTGACACCACAGGAATAAATAAAACCAAAAATGAGGTTCCTA
CTCATTCCCCAAACTTTAGCAATTTTGGTGGTGGTGGTGGTGGTGGTTCGACGTCTTTCCAGAGAAATCCAACAAACATGCCAATTTAGGGAAA
CATCTAGCTCTAGCGACTTTGCCAAACTGGTGTGAGTTGACTCAATGACTGAGTGGATTTCCTTTTTCGGTTTTACTTCACTCAATATTGGTCT
ACACTAAGCTCTTTAGGAAGACAGGGTTGAGAGAGGAACAAGTGTTAATGGGATGTAGATCCTCTGAGGCTGAAAGGAATGTCCTTTGTCTCTA
AACAATGCCGAGGGGCTCAGGAACACTCTCTTCCAGTCTTCACCCCACCATCTCATTAAAGGCTAAACTCTCCAGTTCCGCTCTATATGCCTAG
GAAAGAGGAGCTTTGGGGAAAGCTCTGTCAGAATATACCTGCTTTCTGCAGGGGGCGGGAGGACCCGCACAAACAACTCAGCTGGAGAACAATT
GTTAGATGTGACAATACTGAAGTTTGTACACAGACATGTGAAATTGCTGGCCTGAAGTCACATAAGCACTAACTAACGATAATAGCAATTAATG
TTTACTGTGTGCTAAAAGTTTGACATACATTATCTCCTTTTATTCCCAAAGCAACCCCCTATACACACACACACCTCCTTTTTGGTTTTGGTTT
TACTTTTTAAATTTTACTATTAAATTTTAAATATGTTTTTTTTTTAAATTTACTGCACTAGAGAGGGAACCTAGAACCTCCTGCATGCAAGGTA
AGTGATCAATCCGTGAGTCACATCTCTATACAGATTGTAGGTTGTTTCACCAAAAGTATTAGGGCATTTCCTACAGAGTGGTCGAAGTTCGAGT
CCTGGGAACATGGAATAAGGATGGAATCCAGTAAGTCTTGAGAAGAAGCTAAAGGAGAAAATCTCAGCAGCAGATGGCAGGGAAGCAAAAGAAG
AAGGAGAGCTAAAACCAACAAGCTAAAACCAAAAACGCTTGCTATTTGAACTGTGCCTAAGGTAGCACGAAGTACAGCAAGGATCCACTGGCTT
AGTGAGGGAATCCTTCTCTAGGCCTCTCCTATCCACTTCCCCTTCCACCCACTATCCTCCTACTCCTTCATCTCCTAGAGACAACTGCCCATAG
GTAGTTAATTAGCTGTCCCACCACCCAACTCTCCTGCAGTACTTTGGTTCTGTTTATTTGTTTTGGCAATAGGATCTCATTAGGCAGGCCATAC
TGGCCTCAAACTCTGGAATTTCTTCCCTCAGCCTCTTAAGTGTTCTGTTGGGATTACCACTTTGGGACACTTATTCTCCAGCTTTCATGCAGGG
CTTTATTCAGGAATTTCCACTGTGTGATTCTACGATGCAAATACTAGTTCTCATTCCCTGAGGGTTTAATTGTTGTTGTTGTTTCGTTAGTTGC
TTGGTTTTGGGTTTTGTTCATTGGGTTGGTTTGGGTTGGTTTTGGTTTTTCAAAACAGGGTTTCTCTGTGTCTGAGAACCATTGTCTTTTCTAC
CACTGCAAACCCAACCATCTTTTCAAACCACTGGTAAGCCAAGTCCTTTCTGGATTAAGTCTGAAGCCATTCTCCACTCCTATTCGATACACTT
ACTACCTACCACAAACACGGCACCCTGGCAGCCTCAATGCCAGTTTTGGCCTTCTCCTTGTATGCTTGCAGACCACTTGTCTTCATCATCAGAT
CAACCCCTTGAAGAATGGAAGTTCATTGGTCTTGCTTAATCATGCCTGACAAGAAAGTGCAATTCACTAATTCTGTAAACATCTAAGTGAAGGG
CTCTTTGATGGGCTCAATGGTGCATCAGACCTGAGTAAGGGGTGGAGTAGAAGCAAAGTTGTTTCAAGTTGCATAGCGAGAAAGGTCAAGGAAA
GAGATGGGGGTTCATCAGGTGAAGGGAAGGATTTTGATTACACTTGCTTTGCTAGATTTGGTTTTTCAGCTGAAAAGACAGCAAGCCTAATTCA
TTTCCATGATAGTAGGCTAGACTTGAGAGGGAAAGAACTGCTGACACAAAAGGAGGGGAATTGTACTGACTCGTTTTGTGTGTGTGTGTGTGTG
AGTGTGTGTGTGTGTGTGTGTGTGTGTGTGTGTGTGTGTGAACTTGACACAAGCTCGAGTTATCACAGAGCCTCACTTCGGGAAAAGCCTCCGT
GAGATCCAGCTGTAAGGCATTTTCTCAATTAGTGGTGAAGGAGGGCCTATGTGGGTGGTGCCATCCCTGGGCTGGTAGTCTTGAGTTCTCTAAG
AGAGCAAGCTGAGCAAGCCAGGGGAAGCAAGCCAGTAAGTAACATCCTTCCAAGACCTCTGCATCAGCTCCTGCTTCCTGACCTGCTTGAGTTC
CAGTCCTGACTTCCTTTGCTAATGAACAGCAGTGTGTAAGTGGACAGTGAATAATCCTTTTCTTTCCCAACTTGCTTTTTGCACATGATGTTTT
GTGCAGGAATATAAACCCTGACTAAGACAAGAATCAAAGTGTCTAGTCACGTCAGTTACTGCACCGGTACGTAGCTTTGTTTTGCTTGCTTGAT
GTCTGGAGCCCTCTCTTACCTGCGTATGCCTAAACAAATGAAAAAAGAATATGTTCCCCCTACTTAGGTAACTATTCTTCCAGGTGGTGATGTC
ACTGGCCAAGCTGTTCTCCCTTGGCTTCCCACAGCTCTCTTCCTATACCAGATACCATGTCCATCTTTACACAAATGACTCTAAACCTCTTCTC
TCCACAGACACCTTCAATCCACATGAGATAAAGCCTCCACATTTTGTAGCTCTGGCTGACCTGGAACTCACTATGCAGACCAGCCTTGCATTGA
ACTTTCAGAAATCTGCCTATATCTTTCTTAGAAGTGCTAGGACTAAAGGCATACACCACATTTAGTCCTGCTAAACTTCTTTTTTTGTTTTTTT
GGTTTTTTCGAGACAGGGTTTCTCTGTGTAGCCCTGGCTGTCCTGGAACTCACTCTGTAGACCAGGCTGGCCTCAAACTTACAAATCCACCTGT
CTCTGCCTCCCAAGTGCTGAGATTAAAGATGTGTGGTACTACTGCTTAGACTGACACACAAAATATTGTTGAAGCCAGTGAGATAAAGCTCACC
TTCAATCTCAGAGGTAGGCCAGGCAGACTTTTGTGAGTTCTGGCCCAGCCAGAGCTTCATATGAAAAGTAATAATAATGATAATAATAATAATA
ATTTTAATAAACAAATGGGTGTATGAAAGAGTAAATGTGCACATATTTCTGGCATCTGTTTTAAGATTCTACTTCCTTAGGCTGTCAGTCATTC
TCACCCCATTCAACCCTTTGCTCTGCACTGTGATTGACAAATCTTCTCTCCCTCTGGTGACTACCTAATTCATCCCAGACAGCATCAGCCTTGG
CAACAACTTCTCTATGCAGGGACTGTAAGTTCCAGACAACAGAAACTCTGTTCCAATTCACAAAATCAACTAGGAAAGGAGAAGAGACAGAGGA
CAGAGGAAAGGAAGGAAGAAGGGAGAAAAGGAGGGAGGGAGGGAGCGAGGGAGGGAAAATGTCAATTTATGCAACTGTTTCATGATTCATCCAA
CGATCCTCTCTGATTCTTTTTTCCTTGTTGTTGTTTTTGTTTTTTCGACACAGGGTTTCTCTGTATAGCCCTGACTGTCCTGGAACTCACTTTC
TAGACCAGGCTGGCCTCGAACTCAGAAACCCACCTGCCTCTGCCTCCCAAGTGCTGGGATTAAAGATGTGTGCCACCACGCCCAGCTAAACCTC
TTCCTTCTAGCCAGAAGAACACAGCAGAAAGAACTATGATCAGTCTTCTGGAACCATGTGCTTACCCTTAAACCACACAATTCACTTGGTTCCC
AGGACCACACTCCTCCTGCAAAAGTTGGAACCCTGTCATTCTCAGCTTCTGAGGCAAAGGCAAGACCTATCCAAGATAAAAAACCAACAACAAC
AAAAAAGTCAATATAAGAAAAGAAAAAAATGTTCTCTAGATACATTCTGGGTAGTGATGGTTCTCACTGTTCAGAGATATAAGAAAACACCAGA
TGCTTAAAACCCTGACTCTTGAGAACAACTTTTGATAAGAGACACTCCTTTTTTTAAAATTTTATTTATTTATTTATTTACTTACTTATTATAT
GGTAAGTACACTGGTAGCTGTCTTCAGACACTCCAGAAGAGACTATCAGATCTCATTACAGATGGTTGTGAGCCACCATGTGGTTGCTGGCATT
TGAACTCAGGATCTTTGGAAGAACAGTCAGTGCTCTTAACCACTGAGCTATCTTACCCAGCCCCCCACCCACCCCCCCAACCCCCTTAAGAGAG
ATTCGAATGGTCCATCTTAGAGGTATTGAAATTGAGTCTGAAGTGGGAGGCCTAGGGGCTGTTTTGAGCCAACTCAGCCCAGGTGATTTGCAGT
GAGGTGGGAATTCCAAGGGCAAACAAGCATCAGCAAAAGCTATACCCCTCTACCACAGATTTGCAGAGAATTCAGATGGGAGGCCTGTTCCAGC
CATTGCTCAGTAAGTAAGCAAGAGTCTAGCTACCTCTTCATCTCCCTCCTTCTACCACACCATTAAGTCCCTCCAGGCCATCAGCCTCAATGAT
TCTTTCTTTTTTTTTTTTTAATTAGGTATTTTCCTCATTTACATTTCCAATGCTATCCCAAAAGTCCCCCATACCCTCCCCCTCCACTCCAACT
TTTTGGCCCTGTTGTTCCCCGCCTCAATGCTTCTTGGCTCTATCCATGTCTCCTTGCCCACTTTCCTTCTGCCAACCGATATTCTTTACCTGGG
ACTCTCTAGCAGATGCCAACTCCCTGTCTCTCATCTGGTTTCCTCAAAGGTAGAAAAATGGTCTCCCAAATAAGCCCGATCAGTTTACTCCCTG
GCGTAGGAGAACTTGATGATTCATGTGCTCTGCTTCTAAAATACAGATCTGTGGGCCTGGTAGTGCAGATGTTAATCCCAACTACTCTACTTGG
GAGGCCTAGGGAATGGACTCCATGTTCAAGGTCTGCCTGGACAGCAAGATGAGTGCTGGGGAAGCCAGAGAAAATGAGTAAAACTTTCTCAAAA
GTAAACCATTATAAAAACAAACAAACAAAGCTCCAGAGAACTCGGGAGATGATTGGCTGGTTAAGAGTACTTGCTCTTCTGCATGGGACCCAAG
TTCAGTTCCTAGAAGCCGAGTAGCTTGCACCTTCAATTATGGCCCCCTCTTCTGTTTTCTCAGGACCAGGTGCACACAGTGCATATAACATACA
TGTAAGCAAAATACTCACACACATAAAATTAAAAATAAATAAATCTTTCAGGCAAAACTGACCTGGGATATAGCTCAAGGATAGAGGATTTGCA
TGGGATGCTAGCAGCCCTGACTTCCATCTACAGTCCAGAAAGACAAGTAAATCAGAAATGCCTTTGAGGGACTGAAGAGATGATTCAGAGGTTA
AGAGCACTGGCTGCTCTTCCAGAAGACCTGGGTTCAATTCCTAGTACCTGCATGAGAGCTGACTCCAGTTCCAGGGAATCTGACCCTATCACTC
AGACACTGTTCTTGTCATCTGCACACGGGATGATCACACACATATCATTTCACATGTGGGGATACTGAGAATGAGAGTCAGCAGGTAACTGTCT
CAACTTCAACTAAGCAGGGCAGTTTGTTAGCAGGGATGGAATCTGGATTCTTATTGCCCCCCAGCTTCTTCACTTCCTGCTGCCTCTCTACAAC
CACACCATATTGAAATTACCTTCTTCCCTCTCTTGCTTCCGCACTCAAACTCGTCCTCAATCTAGCTCGCTACCTGACCTCATTCTCTCCTATC
AGATGGATGACTTGCTGTCCTCCTCGGTCATCTCCCGTGTTTAGCAAGACAGGGTATAGAGTTTTAAATGAGTGTAGGCAGAGTGCATGAATCA
TCACACTTTTAGGCAGAGTGCATGAATCATCAGACTTTCCTACGCCAGGGAGTAAACTGAGCACGCATATTTTGGAGACCACTTCTCTACTCCT
AGGGTCAGAAGAGCCTGCAGATGATCTACTCAAAGCAATTTTATCTGCCATTCTGTTGAGTAGTGAGGCAAAGATCATGTTACCTGCAACGGGG
CCAATCTTTGCCTCTGTTTTTGAAGTCTCTCGACAAGTAGCCGTCTTCCTGCGCTGGGGATATGATTAAGCAGTACAACATGCACATAATTTCA
TCAGGCTAACATACAGC1CTTCAGAGATCACCCTACTTATTTATGACACAATTGTTTTAAGACATCAGGTATTCAAGGAGATAAGATTCCTTCT
CTTGCTTTCCTGGTACCCAGAATGATCTCCTAAACCACAAGTAATTGGCACAGAGATGGATCTTCCAAACTGACCCACATCTACACAGATCTTA
TCTTCATACCCAAGAACTCCCCAGTCAATGGATATTTGGGGCATTTGCTATGCACACAGACGTCAGTGTGGAATAACCGTACTCCAGCACTTAG
TAGAAAGGATGTATACTGAACGAGTATTTACAAATAATTAACCAGATTATTTTCAGGGCACAAATTAGTGGAGCAAGGATTGCAGAGTTACATG
AACATCTGATAGCATTGCGGTTCAGGGAAAGTTCCCAGCCAGGGTACAGAGGATGGTGATGAAGAAAGCATGCTCAATATCTTGAGGCGCTGCT
ATGAGAGAGAATGAAACTCTCTAGCGGAACTGAAAGCATACAAGGATGTAAAGAATACGAAGAGCAAAAGGACTTCAACCCTTGGATGAGACAC
AGGGTGATCCAATACCCGGTGACCTGCAGGGACTCAGGCCCAGTGGTTATGATGCCAACCTACCCACAGTCGTGAGTATCATCCACTGGGAAGT
CACAGTACTGAAAACCAACACACTTAACTTGCCTGTGCTGCTGTGAACGTCCATTCAATGAGGGTCAGGAAGCTCAAATAAGATAATCGCAAAT
ACTGAGTGGTTCTGAGAGGAATGCAACCATGAAAAAAAAAGTGCTGACACTTGCAGAATGTTTATGTTTCTAATGCTGGGCTAAGTACTTCACC
TCGATTATTTCACCAATTCCTCCTCCTCCTCTCCCTGTTCTCCCTTCTCCTCCTCTTCTTCCTGTTCTTCCTCCTCCTCTTCCTCTTCTTCCAA
AGATTCATGTGTATCAAGGTATTCTCAA
MOUSE SEQUENCE—mRNA
GATTGGGGTACCCGCTGCGCCAGGTTTCCGCACCAACCAGAGCTGGATAACTCTAGACTTGCTTCCCTTGCTGTGCCCCCTCCAGCAGACAGCC
ACGACGATGCCCCTCAACGTGAACTTCACCAACAGGAACTATGACCTCGACTACGACTCCGTACAGCCCTATTTCATCTGCGACGAGGAAGAGA
ATTTCTATCACCAGCAACAGCAGAGCGAGCTGCAGCCGCCCGCGCCCAGTGAGGATATCTGGAAGAAATTCGAGCTGCTTCCCACCCCGCCCCT
GTCCCCGAGCCGCCGCTCCGGGCTCTGCTCTCCATCCTATGTTGCGGTCGCTACGTCCTTCTCCCCAAGGGAAGACGATGACGGCGGCGGTGGC
AACTTCTCCACCGCCGATCAGCTCGACATGATGACCGAGTTACTTGGAGGAGACATGGTGAACCAGAGCTTCATCTGCGATCCTGACGACGAGA
CCTTCATCAAGAACATCATCATCCAGGACTGTATGTGGAGCGGTTTCTCAGCCGCTGCCAAGCTGGTCTCGGAGAAGCTGGCCTCCTACCAGGC
TGCGCGCAAAGACAGCACCAGCCTGAGCCCCGCCCCCGGGCACAGCGTCTGCTCCACCTCCAGCCTGTACCTGCAGGACCTCACCGCCGCCGCC
TCCGAGTGCATTGACCCCTCAGTCGTCTTTCCCTACCCGCTCAACGACAGCAGCTCGCCCAAATCCTGTACCTCGTCCGATTCCACGGCCTTCT
CTCCTTCCTCGGACTCGCTGCTGTCCTCCGAGTCCTCCCCACGGGCCAGCCCTCAGCCCCTAGTGCTGCATGAGGAGACACCCCCCACCACCAG
CAGCGACTCTGAAGAAGAGCAAGAAGATGAGGAAGAAATTGATGTGGTGTCTGTGGAGAAGAGGCAAACCCCTGCCAAGAGGTCGGAGTCGGGC
TCATCTCCATCCCGAGGCCACAGCAAACCTCCGCACAGCCCACTGGTCCTCAAGAGGTGCCACGTCTCCACTCACCAGCACAACTACGCCGCAC
CCCCCTCCACAAGGAAGGACTATCCAGCTGCCAAGACGGCCAAGTTGGACAGTGGCAGGGTCCTGAAGCAGATCAGCAACAACCGCAAGTGCTC
CAGCCCCAGGTCCTCAGACACGGAGGAAAACGACAAGAGGCGGACACACAACGTCTTGGAACGTCACAGGAGGAACGAGCTGAAGCGCAGCTTT
TTTGCCCTGCGTGACCAGATCCCTGAATTGGAAAACAACGAAAAGGCCCCCAAGGTAGTGATCCTCAAAAAAGCCACCGCCTACATCCTGTCCA
TTCAAGCAGACGAGCACAAGCTCACCTCTGAAAAGGACTTATTGAGGAAACGACGAGAACAGTTGAAACACAAACTCGAACAGCTTCGAAACTC
TGGTGCATAAACTGACCTAACTCGAGGAGGAGCTGGAATCTCTCGTGAGAGTAAGGAGAACGGTTCCTTCTGACAGAACTGATGCGCTGGAATT
AAAATGCATGCTCAAAGCCTAACCTCACAACCTTGGCTGGGGCTTTGGGACTGTAAGCTTCAGCCATAATTTTAACTGCCTCAAACTTAAATAG
TATAAAAGAACTTTTTTTTATGCTTCCCATCTTTTTTCTTTTTCCTTTTAACAGATTTGTATTTAATTGTTTTTTTAAAAAAATCTTAAAATCT
ATCCAATTTTCCCATGTAAATAGGGCCTTGAATGTAATAACTTTAATAACGTTTATAACAGTTAAAAAAAAAAAAAAAAAAAAAAAAAAAAAAA
AAAAAAAAAAAAA
MOUSE SEQUENCE—CODING
ATGCCCCTCAACGTGAACTTCACCAACAGGAACTATGACCTCGACTACGACTCCGTACAGCCCTATTTCATCTGCGACGAGGAAGAGAATTTCT
ATCACCAGCAACAGCAGAGCGAGCTGCAGCCGCCCGCGCCCAGTGAGGATATCTGGAAGAAATTCGAGCTGCTTCCCACCCCGCCCCTGTCCCC
GAGCCGCCGCTCCGGGCTCTGCTCTCCATCCTATGTTGCGGTCGCTACGTCCTTCTCCCCAAGGGAAGACGATGACGGCGGCGGTGGCAACTTC
TCCACCGCCGATCAGCTGGAGATGATGACCGAGTTACTTGGAGGAGACATGGTGAACCAGAGCTTCATCTGCGATCCTGACGACGAGACCTTCA
TCAAGAACATCATCATCCAGGACTGTATGTGGAGCGGTTTCTCAGCCGCTGCCAAGCTTGTCTCGGAGAAGCTGGCCTCCTACCAGGCTGCGCG
CAAAGACAGCACCAGCCTGAGCCCCGCCCGCGGGCACACCGTCTGCTCCACCTCCAGCCTGTACCTGCAGGACCTCACCGCCGCCGCGTCCGAG
TGCATTGACCCCTCAGTGGTCTTTCCCTACCCGCTCAACGACAGCAGCTCCCCCAAATCCTGTACCTCGTCCGATTCCACGGCCTTCTCTCCTT
CCTCGGACTCGCTGCTGTCCTCCGAGTCCTCCCCACGGGCCAGCCCTGAGCCCCTAGTGCTGCATGAGGAGACACCGCCCACCACCAGCAGCGA
CTCTGAAGAAGAGCAAGAAGATGAGGAAGAAATTGATGTGGTGTCTGTGGAGAAGAGGCAAACCCCTGCCAAGAGGTCGGAGTCGGGCTCATCT
CCATCCCGAGGCCACAGCAAACCTCCGCACAGCCCACTGGTCCTCAAGAGGTGCCACGTCTCCACTCACCAGCACAACTACGCCGCACCCCCCT
CCACAAGGAAGGACTATCCAGCTGCCAAGAGGGCCAAGTTGGACAGTGGCAGGGTCCTGAAGCAGATCAGCAACAACCGCAAGTGCTCCAGCCC
CAGGTCCTCAGACACGGAGGAAAACGACAAGAGCCCGACACACAACGTCTTGGAACGTCAGAGGAGGAACCAGCTGAAGCGCAGCTTTTTTCCC
CTGCGTGACCAGATCCCTGAATTGGAAAACAACGAAAAGGCCCCCAAGGTAGTGATCCTCAAAAAAGCCACCGCCTACATCCTGTCCATTCAAG
CAGACGAGCACAAGCTCACCTCTGAAAAGGACTTATTGAGGAAACGACGAGAACAGTTGAAACACAAACTCGAACAGCTTCGAAACTCTGGTGC
ATAA
HUMAN SEQUENCE—GENOMIC
ACTCCCACCACACCTATTCAACATAGTATTGGATTCCCTGGCCAGAACAATTAGGCATGAGAGAGAAACAACAGGCATCCAAACAAGAAGAGAG
AAGGTCAAATTATCCCTGTTTGCAGACAATATGATTCTGCATCTAGAAAACTCCATAGCCTCTGCCCCGAAACTCCTTGAGTTGATAATCAACT
TCAGCAAAGTTTCAGGATACAAGATCAATGTACAAAAATTAGTAGCACTCCTATACACTAACATCACCCAAGCCAAGAGGCAAATCAGGAACAC
AATCCCATTCACAATTGCCACAAAATAATAAAAAGCTTAGGAATACAGTTAACTAGCGAGGTGAAAGATCTCTACTATGAGAATTAAAAAACAC
TCTTCAAAGAAATCAGAGATGACCCAAACAATTGGAAGAACATTCCATGTTCATGGAAGGGAAGAATCAATATTGGTAAACTGGCCATACACCT
CAAAACAATTTACAGATTCAATTTTATTCCTATCAAACTACCAATGACATTCTACACAGAAATGGAAATAACTACTCAAAAATTCATATGGAGC
TAATAAAAGAGTCTAAATACACAAGCCAATCCTAGGCAAAAAGAGCAAAGCTGAAGGAATCACATTGACCAACTTCAAATTGTACTACAAGGAT
GTAGTAATCAAAACAGCATGGAACTGATACCAAAACAGACACATAGACCAATGGAACAAAATAGTGAGCACAGAAATAAAGCCACATACCTACA
ACCATTGGATCTTTGACAAATCTGACAGAAACAAGCAAGAGGGAAAAGACTCCCTATTAAATTAATGGTGCTGTGATTACTGGTTAGCCATATG
AAGAAAACTCAAACTAGACTTATGCCATATATAAAATCCATATGCCGGAAAGCTGAGGCAGGAGAATCACTTGAACCCGGGAGGTGGAGGTTGC
AGTCAGCCGAGATCACACTACTGCACTGCAGCCTAGCGACAGAGTGAGACTCCGTTGCAAAAATAAATAAATAAATAAATAAATAAATAAATAA
ATAAATATCAACTCAAGGTGGATTAAAGACCAAAATGTAAAACTCTAAAACTATAAAAACCCTGGAAGATACCCTAGGAAATACCATTTTGGAC
ATAGGAGCTGACAACGATTTTTTGAAAAAAACACCAAAAGCAAAAGTTGACAAATAAGACCTAATTAAACTAAAGAACTTCTGCACAGCAAAAG
AAACTATCAACAGAGTAAATAGGCAACCTGCAGAATGGAGGAAAATATTGACAAACTATATATGTGACAAAGGCCTAATATACAAAATCTACGA
GGAACTTAAACATACAAGGAAAAATCAAACAACCCACCTCACTAAAAAGTGGGAAAAGGACATAAACCGACACGTTTCAAAAGAAGACATACAA
GCATATACATACAAGCCAACAAGCATATGAAAAAATGCTCATCATGACTAATCATTAGAGAAATGCAAATCAAAGCAGCAATAAGATACCATCT
CACACTAGTCAGAATAGCTATTATTAAAAAGTTAAAAAATCACAGATGCTGGTGAGGTTGTGGACAAAAGGGAACGCTTATACACTGCTGGTGG
GAGTGTAAATTAGTTCAACCATTGTGGAAAGCAGTGTGGTGATTCCTCAAGTAATTAAAAACAGAACTACCATTTGACCCAGCAATCCCACTAC
TGGATATAAACCCAAATAAATATAAATCTTTCCACCACGAAAACACATGCAAGCATGTGTTCATTGTAGCTCTATTCACAATAGCAAAGGCATG
AAATCAGCCTAAATGCTCAACTACTGCAGACTGGATAAAGAAAATGTGGTACATACACACGATGCAATACTAAGCATCCATTAAAAAAAAAAAA
GAAATCATGTCCTTTTCAGGAACATGAATCGAATCAGAGGCCATTATCCTTAGCAAACTACCACAGGAACAGAAAACCAAACCTATGTTTTCAC
TTATAATTGGGAGCTAAATAATGATAACCCATGGGCAGAAAGAGGCCTCACACTGTGAGGCCTGCGTGAAGGAGGAGGATGGGAGGAGAAAGAA
CTCCAGGGGAAAAAAACTTCGGGTACTGTGCTTAGTACCCAGACAGCAAAATAATCTGTACACATTGATGGGACGTATCTCAAAATAATAAGAG
CTATCTATGACAAACACACAGCCAATATCATACTGAATGGGCAAAAACTGGAAGCATTCCCTTTGAAAACTGGCACAAGACAAGGATGCCTTCT
CTCACCACTCCTATTCAACATAGTGTTGGAAGTTCTGGCCAGGGCAATCAGGCAGGAGAAAGAAATAAAGGGTATTCAATTAGGAAAAGAGGAA
GTCAAATTGTCCTTGTTTGCAGATGACATGATTGTATATTTAGAAAACCCCATCGTCTCAGCCCCAAATATCCTTAAGCTGATAAGCAACTTCA
GCAAAGTCTCAGGATACAAAATCAATGTGCAAAAATCGCAAGCATTCTTATACAACAATAACAGACAGAGAGCCAAATCCTGAGTGAACTCCCA
TTCACAATTGCTTCAAAGAGAATAAAATACCTAGGAATCCAACTTACAAGGGATGTGAAGGACCTCTTCAAGGAGAACTACAAAACACTGTTCA
ATGAAATAAAAGACGACACAAACAAATGGAAGAACATTCCATGCTCGTGAATAGGAAGAATCAATATCGTGAAAACGGCCATATTGCCCAAGGT
AATTTATAGATTCAATGCCATCCCCATCAAGCTACCAATGACTTTCTTCACAGAATTGGAAATAACTACTTTAAAGTTCATATGGAACCAAAAG
AGAGCCCGCATTGCCAAGTCAATCCTAAGCCAAAAGAACAAAGCTGGAGGCATCACGCTACCTGACTTCAAACTATACTACAAGGCTACAGTAA
CCAAAACAGCATGGTACTGGTACCAAAACAGAGATATAGACCAAAGGAAGAGAACAGAGCCCTCAGAAATAATGCTGCATATCTACAACCATCT
TACCTTTGACAAACCTGACAAAACCAAGAAATGGGGAAAGGATTCCCTATTTAATAAATGGTGTTGGAAAACTGGCTAGCCATATGTACAAAGC
TGAAACTGGGTCCCTTCCTTACACCTTATACAAAAATTAATTCAAGATGGATTAAAGACTTAAATGTTAGACCTGAAACCATAAAAACCCTAGA
AGAAAACCTAGGCAATACCACTCAGGACATAGGCATGAGCAAGGACTTCATGTCAAAAACACCAAAAGCAATGGCGACAAAAGCCAAAATTGAC
AAATGGGATCTAATTAAACTAAAGAGCTTCTGCACAGCAAAAGAAACTACCATCAGAGTGAACAGGCAACCTAAAGAATGGGAGAAAATTTTTG
CAATCTACTCATCTGACAAAGGGCTAATATCCAGAATCTACAATGAACTCAAACAAATTTACAAGAAAAAAACAAACAACCCCATCAAAAAGTG
GGTGAAGGATATGAACAGACACTTCTCAGAAGAAGATATTTATGCAGCCAAAAGACACATGAAAAAATGCTCATCATCACTGGCCATCAGAGAA
ATGCAAATCAAAACCACAATGAGATACCATTTCACACCAGTTAGAATGGTGATCATTAAAATGTCAGGAAACAACAGGTGCTGGAGAGGATGTG
GAGAAACAGGAACACTTTTACGCTGTTGGTGGGACTGCAAACAAGTTCAACCATTGTGGAAGTCAGTGTGGCGATTCCTCAGGGATCTAGAACT
AGAAATACCATTTGACCCAGCCATCCCATTACTGGGTATATACCCAAAAGATTGTAAGTCATGCTGCTATAAAGACATGTGCGCACGTATGTTT
ATTGCGGCACTATTCACAATAGCAAAGACTTGGAACCAACTGAAATGTCCATCAATGATAGACTGGATTAAGAAAATGTGGCACATATACACCA
TGGAATACTATGCAGCCATAAAAAATGATGACTTCATGTCCTTTGTAGGGACATGGATGAAGCTGGAAACCATCATTCTCAGCACACTATCTCA
AGGACAAAAAACCATACACCACATGTTCTCACTCATAGGTGAGAATTAAACAATGAGAACAGATGGACACAGGAAGGGAAACATCACACACCGG
GGCCTGTTGTGGGGTAGGGGGAGCGGGCATGGATAGGATTAGGAGATATATCTAATATTAAATGACGAATTAATCAGTGCCCCACACCAACATG
GCACATGTATACATATGTAACAAACCTGCACATTGTCCACATATACCCTAAAACTTAAAGTATAATAAAAAAAATCTGTACACTAAAACTCCAA
GTCATGACTTTACCCATATAAAAAACCTGAACCTAAAATAAAAGTTAAAATATTCAAATATAAATAAGTAAATAAATAAAATGACAAACCCATG
GCTAACATCATGTTGAATAAGGAAAAGTTGAATGCTTTTTTTTCTAAAATCCAGGAGAAGACAAGGATGTCCATTCTCTCTTCTACTCAACATG
GTACTCGGAGTCTTAGGTAGAACAATTACACAACAGAATCAAATAAAGGTCATTCAAATTGCACACCAGCAACTCCTTTTCTGCAGTAACATGA
TCTTATACGTATAAAACCCTATACACTCAACCAAACAAAAATAACTGGTAGTATTGATAAATCAATTCACTAAAACTTCAAGTTACAAATTTAA
TGTACAATAATTAGTAGTGTTTCTACACAGCAACAGCAAACTATCTGAAAAAGAAATCAAGAAATCTATCTCTTTTACAATAGCTACAAAAATT
TCAAAAAATTTCAAAAAATACCTAGAAATAAATTTAACCAACGACGTGAAAGATCTCTACAGTAAAAAATAATAAAATATCGATGAAAGAAATT
GAAGAGGATACTAATAAATGTAAAGTTAACCCATGCTCATAGATTTGTAGAGTTAATGTTGTTAAAATGTCCATACTACCCAAACCAATGTACA
GATACAATGCAATCTCTATCAAAATACTAAGGACTTTCCTCACAGAAGTAAAATATATATATTTTTTAATATATAAAATGTATATATAAATATA
ATATATAATATGAATATTATATATGATTATAATATTATATGATTATATACTATTAATATTATTAATAATATATATTATTATATATATTATATAT
ATTATTATATATTGTATATAAATATATATTATATACTATATTATTATATATTGTATATAAATATATATTATATATACTATATATTTATATACTA
TATATATAGTGTATATATATATTTATATAGTATATATAATATATACACTATATATTTATATAGTATGTATATATATATACTATATATAATACAT
AAAATATATATACATATATAGTATATATATGAAATATATATATACTAAATTTGCACAGAACCACATGATTCCAAACAGCCAAAGCAATCTGGAG
CACAAAGAACAAAGCTAGATGCATTACACTACCTGGCTTTTAAATTTTCTACAAAGCAATAGTAAACAAAATAGCATAGTACTAACATGAAAAC
AGATCCATAGACTGATGGAGGAGAATAGAGACCCCAAAAATGAATCCATACAATTATACCCAACTAGTTTTTCACATATCTACCAAGAAAACAC
ATTGGGGAATGGAAAGTCTCTGCAATAAATGGTGCTGGATAAACTGAATATCCACAAGCAGAAGAAAGAAACTAGCTGCTTCTCTCTCCCAACA
TATAAAAATCAACTCAAAATGGATTAAAGACTTGAATATAAAACATGAAACTAAGAAGATCCTTGAAGAAAACACAGGCGAAACAATTCATGAT
ATTGGTCTGGGAAAGAATTTTTCAAATAAGACCTCAAAAGCACAAACAACCAAAGCTAAAATAGATGAATGGGATAATATCAAGCCAAAAAGCT
TTTACATCTTAAAGGAAACAACAGAATGAAGAAATGACCTACAGAATGAAGAAATATTCACATACTATCCATCTGACAAGAGCTTAATATCAAA
CTATGTAATCAATGCAAGCAACTCAATAGGAAACACACAAATAATCCAATCAAAACTTGGGCAAAGGATTCGAATAGATATTTCTTAAAACGAC
TCACACAAATGGGCACCAAGTATATCAAAAAAGGCTCAACATCACTAAGAACCAGGGAAATGCAAATCAACACCACAGTGACCTATCCACTCAT
CCCAGTTAGAATAGTTATTATTTAAAAAAATAAAATAAAAAGACAAGAAAATGGCCAGTCATGGTGGCTCATGCCTGTAATCCCAGCATTTTGG
AGGCCAAGGAGGGCAGATCACTTAATGCCAGGAGTTCAAGAACAGCCTGCCCAACCTGACGAAACCCCATCTCTACTAAAAATACAAAAAATTA
GCTGGGCATTGTGGCACATGCCTGTGGTCCCAGCTACTCAGGAGGCTGAGGCACGAGAATTGCTTGAACTGCGGACGCAGAGGTTGCAGTGAGC
TGAGTTCGCACCACTGCACTCCAGCCTGGGTGCCAGAGCAAGGATCTGCCAAAAAAAAAAAAAAAAAAAAAAAAAAAAAGACAAGAAATAACAT
ATGCTGGTAAGGATGTGGAGAAAAGTGAACTCTTATACAATGCTATACACTTGGTGTACTACTATATTATCCAGCAATCCCACTGCTGGATCCC
CCTCCCCCCCAAAAATGAAATCAACATATCAAAGAGTTCAGATCCCCATGTTTATTGCAACACTATTTGCAATAGCCAAAATATGGAATCAACT
TAAGTGCTCATCAACAGATGAAACAGGAATGAAAATATGGTATATATACACAATGGAATGCTATTCAATCTTTAAAAAAGAATGAAACCCTCAA
AAAGTGGGTGAAGGACATGAACAGACACTTCTCAAAAGAAGACATTTATGCAGCCAAAAAACACATGAAAAAATGCTCACCATCACTGGCCATC
AGAGAAATGCAAATCAAAACCACAATGAGATACCATCTCACACCAGTTAGAATGGCAATCATTAAAAAGTCAGGAAACAACAGGTGCTGGAGAG
GATGTGGAGAAATAGGAACACTTTTACACTGTTGGAGGGACTGTAAACTAGTTCAACCATTGTGGAAGTCAGTGTGGCGATTCCTCAGGGATCT
ACAACTAGAAATACCATTTGACACAGCCATCCCATTACTGGGTATATACCCAAAGGACTATAAATCATGCTGCTATAAAGACACATGCACACGT
ATGTTTATTGCGGCACTATTCACAATAGCAAAGACTTGGAACCAACCCAAATGTCCAACAATGATAGACCGGATTAAGAAAATGTGACACATAT
ACACCATGGACTACTATGCAGCCATAAAAAATGATGAGTTCATGTCTTTGGAGGGACATGGATCAAATTGCAACTCATCATTCTCACTAAACTA
TCGCAAGAACAAAAAACCAAACACCACATGTTCTCACTCATAGGTGGGAATTGAACAATGAGAACACATGGACACAGGAAGGAGAACATCACAC
TCTGGGGACTCTTCTGGGGTGGGGGGAGGGGGGAGGGATAGCTTTAGGAGATATACCTAATGCTAAATGACAAGTTAATGGGTGCAGCACACCA
GCATGGCACATGTATACATATGTAACTAACCTGCACATTGTGCACATGTACCCTAAAACTAAAAGTATAATAATAATAATAAAAAAGTAAAAAT
GATTAAGGACTATAATAGGATAAGAACTCTAGGAAGATGATAAAGTATGCCCAAAAGAAAGCAATGCTATAATAAAAACAAAATAAAACAAAAC
AACAACAACAAAAAGAATGAAACCCTGTCATTTGCAACAATGTGCACAAACCTAGACGACTTTATCTTAAGTCAAATAAACCAAGCACACAACG
AGAACTACCACATGATCTCAGGCATATGTGGCATCTAAAAAAGTTGACCTCAAAGAGAGTTTAGTAGTGGCTATCGGAAACTGAGGAGAGTAGT
TGCGAGGGGGAGGGGAACAGGTTGCTCAACAGCTACAAATTTACACTTATATAGGACAAATAATTTCTGATAATCGACTGCACAGTACCTTCAG
TCTAGTCAACAATAATGTATTGTGTACTTTAAAATAGTTAGACGAGACGATTTTGGACGTTCTCACCACCAAGAAATGACAAATGTTTCAGCTG
ATACACATATGCTAATTATGCTGATTTGATCATTACACAATATATATGTATCAAAACATCACACTGTATCCCATTAATATGTACAATTATACAT
CAATTTAAAACAAAATAAAATGTATTTTATAAAAATTTTAAAACTTCAACCAGCAAGTTTGAATCTCCTATTCCCAGTTTTGGTCAGCCCTACA
GTGTATACCAACCAGGCTGGCCTGGGCCTGCTGTTAGAGGAAAGAAAGAAACATCTGGACTTGGAATGAACAGATCTGGAAGGAATCCTAAATT
TTCCAATAAAGTCTCTGTATTCCTTTCATCCTGGTCCCTACCCACGAAGCAGTCTAGAGCAGATGATCCTTTTCTAAGAACAGCAACATTCAGT
AAACAAACTTCCTTACTAATGTCTGTGGATCAAGAGATGGTGAGGAAAGTAGAATCAGTGATATTTTTGAATCAATTATCAGAAGGCCACCATG
CTATTGCTCAGTAATATTAGATGCCCATGGACAGTAAGATTCATCGATGGTCCGTCTTTATTATTAGGTCATTACTGAGTGGCTTTTGCAATAA
AATACACCATTGGCAGAATAAAAATATTCTCTAATGCAAAAACATAGCATGGATTGTTTTTAAGGGGCACAATAGTGTGCTTGATCAGACTGGA
ATAGCAACATTGTAATCTGAAAAAAGCTCAACAGTCGTCAGCCATATTCAAAAGCCCCAAGAGGAGAGTCAACAGTCAGATCCATCAGAGTTGA
GAGGTTGCCACAACTTATGTAATGACGTCTCCACGCCATGAAACACATGTGTCAGCTTTTGTCAGGAGTCACAGATCTGGCATGCAGCAGCAGA
TTCTGCATTAGATATATAAAGTCAGGCCCAATCATCCCCTTGATTAGTTAGTCTCATCAGAGAATGAGCCCTTCCCATCGGTCTGTATATGAGG
AATCCAGGCAGTTCACTAAGTAGTCAAGATTTGTTTCCACATGCGGTCCCAAAGAAGTCTTTAATCTGACAGACTAGTTTTCCAAGTGGCATAC
CAAAAGGCTGGACCATTGGTAGTGTCCCAAGAATTGGGTTCTGACCATTGAGCAGAGAAACCAAGTCCTCTTTTTATTTCTGCATAGCTGGTTC
AGACGCTGAACAGACACAGGAGCCAGTGAACACCATCAGTTTTCAGCTTAGCCAATCCTTCACTGAGGCAAGCCCAGATATTTGGGCAAACCTC
TGTAAACAGGGTCCAATTGAACTAGTAGTGTTGTTTCAAGCATCAGAATAAAAGGTTAAGTTTCCCCCAGAGAGACAGTTGCCACTCTTACATA
TAAAGCAGAGATGCAGAAGAAATTCCAGTTGATGCCATCGCCCACCCACCAACCCACCCCCGCATTTCCGCCCTGAGACTAGGATCCCAGCGCC
AGGGACCCAGACCCTGGGATTGATCGCACAGAGCCGTGATCCGCAGAGTGCGCCGCGGATGAGACGGAATCTCGGGGCCACTCGAGAGCTTCGC
GCAGGAACCATGCATCCATTGTCCATCGTGACTGCATCTCCAGCAGCTATTTGGCCTGGGAACAGCTGTAGAGAGCGGGGAGCGACTGGCTCGC
CACTGGCGCGCCCTGGCGGAACCCGCCCAAGGTGCCCTCCTCCCCACACTGCCCTCCCAGCCTCGAAGACGGCAATCCAGCCCCCACTCCTCCC
TGGACTCTGGGCAAGGCCAAGACTCAGGCCTCCTCCAGATTTGAGAACCCAAACAACTCAGAGGGTGACGAAATTGACCTTGTGACAGCAAAGA
AAAGGCAGATTGGGTTTACAGAAGTCGATCACCATCATGGTTCGAGTAGACCGTCTGAACCCCTCCATCAAACACTTCCATACCTCCATCCACC
AGCAACAACACAACTGCGCTGCCCATATTTCTCCAGAAAGCTGCTCCCAAGAAGAGGCTCCAGAGAGAGGTCTCCAAGAACAGCTTCTGCAGCG
AGATGCTCCAGGGGAAACGGAAGATGAGGAGGATGAGGGGATTGTGAGCCCCTCACCTATAGATAGCAAGGCTGCCCAGTGCTGCCAGTTCTGA
CACTGAGGATGTGACCAAGAGGAAGGGCCACAGCTTCCTGCAGCACAAGAGGTGGAATGACCTGCCCGTTCTCACTTCTTGGCCCTGAGCGACC
AGGTACCCACCTTGGCCAGCTGCTCTAAGGTCCCCACGGTAGTGATCCTAAGCAAGGCCTTGGGATACTTGCAAGACCTGGCGTGAACCGAGAA
AAAGATGTCTATGGAGAAAACGCAGCTCCAACGCCAGCAACAGCAATTGCAGAAATGAATTGCGTACCTCAGTGGCTACTAACTGACCAAAAAG
CCTGACTGTTCTATCTTACAAAGACACAAGTTTATTTTTTCACCTCCCCTTCCCCTTTAGTAATTTTCACATTTTGGTTATGACGGCACAGTCT
TTGCAGTAGGTCCCAGAATGCATTGCAGCCAGTACACACAGAATAAGGGCTTGCATTCTTGGAAACCTTGAAACCCAGCTCTTTCTCTCCCCTG
ACTCATGCTGTGTCTTCTCTGGCGCCTTTGGCTTCTCAGCAGATACCTCACTGAGGAGATTTGGGGTCTGTTTACCTCACTAGCTCCGAAGAAA
AGGCTGACAGATACTATGCAACAGGTGGTGTATGTTGTCCGGCACTCCAGCCTGCATGAAATCTCACACTCCGCGTGAGCCTTAGACTAGGAAA
GAATGCTCCCTCGTATCTCTGGGGTGATGCTAGAATCCTCCCTGGTATGTCTGGGGTCATGCAAGGACAGCTGGGCCTGGACAGCACTCTCGCT
GTGGCTTTTTTTTCCAGGAGACACACAAGCTGTCTTGGGTGATGACAAGCTTGAAGATTTGATCAACATGACCATTGCTTCACTGTCAGACACT
TTACAGTATCTGAGGAGTTGGAAACCTTTAACGTATATATTGTGATATTAGCTGACACCTCTCCTTCCAGTTTCAATGCTGAGACCCTGAGAAC
ATTTAAAGAGCTTGCACTCTAGGTTCGTTGTCTCAGAGCTCTCTGGGCCTTCTCTGATGAAGGGACCTTTCTGTCCTCATGAGAGACTTTTGTT
TCATTTTGCCTTTGTTGTGCAATGGGCTTTACAGCATCCTTTCCCACAGGTTAGAAATGTTTCCCCAAGTTACAGGGAAGTGGGGATCCTAGCC
TGGCGCCTGAGGAAATCTTCGAGTCCTGGCTCCTCAACTTCTTCCCTGTCCGAGTCGCACTTGACCCCACCCATCTTATAATCTCTTCTCAAGC
CAGATGTAAGTCACCTCAGAAGGGAGAACTGTACCGCTTCCTCTTTTCCATGAAGCTCTCATCTCAATCTTTGACTGATGAGTGTGAAATTCTA
CCGGAACCATGCAAACACGTCCACCTTGGGCATCTCCAAGGAACTTCTGGGGTCTGCAGCATACTTGGCTCCCTACCACCCTCCCATGAGTCAT
ATTTCTTTTCCAGAAGGTGGACTTGCTTCCTGGTATGTTTTAAAGGAGCCTGCAGGAGCTCTGCTTAGCCAATCATGATGGATTTTTGCCCCAG
CTGGACTCTGCATGTCCAAGGAGAATCCAGGTACAGCCTCCATTCCGGGAAAGACATCCAGCCCAGCAGTTGTCATGTGGGTAACCTCAGGAAC
CCCTAACCCTGTCCTGGAAAAAGGACAAGCCCCTCCAGAACTCTGCCCAAAATAGCAGGTGCTTGATGGTTCTGAATTTGGAAACCGATGGCGC
GTGATAAGTACTATCTGTGGCTCTGGAAAACCAGCTGCTACATTCAAATCTATTTTCCATAATGGTTTCTTTCTGAGGTTGCTTCGTGGCCTCA
GAGAATCCCAGAGGATGTTTTGAAATAGCCTCTCTACCCTTCAGGAGCATGGTAGTTTACAGGAACCAACTGACTTCTGGAACTGTCTATGGAC
GGAGAACAGCCCAGGTGTAGGTTACTAATGTCCTACATGAATAGCTTGGTTTTATAAGCTGCTGTTGGCTATTATGTTCGGGCAGTCTTTTTAA
TATATTGTATTTTTGTACGCATTTTGCAAAGTGGAGTTAACTGTTTTGTAAAAGAAAAAAAAGAAACCCTTGCGTCCTTCTTCTGTTTCAAGGG
TCTGATTTATTTGGAAAGGCAAGTTTATCTGAAATTTTGTATTATTGATTGCCCAATTTTAAAATGTTCCCTTCTGGGACATCTTGACAAAAAA
TATTTCTCAAACATGAAGAAAATAAATATAATAAAAAAGAAATGTCTGTCGGATCAGATGACTGTTCTTGATTTTACCGTTTCCATTTCATAAG
CCAAGCTTATTGACCCCTTCCTACCTGCTAGTCATTGAGTCTGAGTTGACCAACTCAAAGATGGAAATATCGAGCCAGATGGTAACAAATTCTC
CATGAATCGATTGTTCAGTTTTGACAGAATCCCAATTGCAAGCCATTAGCTGCTTTTCAAAAGGGATGTACCCTGTAGCCATATTAGCGAGGCA
ACAAGTCCAGTTCCCCAAGGGATCTCTCTGGGTGTTGACTGCTTCCCCTTGCCAGGTGCCCCCATCGGCAAAATCATCAGTTACACAGATTTGT
CCTCTATGTAATATTCAACCAGTTGATTCAGCTTCTGAGCCTCTTTTTCGGTGGCAGGTTGTATGAAGAGACGTCAATTTTTTCCTGTTACAGC
GATCTAATGTTGTGAATTTGCCTGCTTAGTCCCAAGAGATTACTTGGCAAGCAGGCCCCTGTATTTGGGAGCATAAATCAGCCACCCTTGCTGA
CAGATATGTAATAACACTGCATTGAGGACCCTTGAGACTGAGACTTCTAGCCGGCCAGCTGACACAATTATTATGTATATAGTGAAAATATTGA
ACATTAGAAGGTAGAGACACTAATGCTAAATCCTGAGCCAACCACTGCTGACAAATCTCATTGGAGTTTGTTCCACTAACACTTCCCTCTACCT
CTGTCTCTCTGTGATGGGGATCACTTTATATGAGACCTGTAGAATGAGAAATTACAAGCATATGACCCATCAAATCCTGCCATTTATAAGTAGA
AGCAACAAGAACAGTTTATTCAACTTTCCCAAAGGCGTCCCTGTACAAAGTCATATTGGCTTCCAAACCCCACTGTGAACCATGCCCAAACCCC
AACAATTGCTTTTCTGGACTTTTTTCCCTTAAATGAACTCAGACCTAGAGTTCACTACAGCAATTTCAGGCGTTACACCAACAGCTGTTAGGGG
ATTTCCCTATGTCCAGGCCAGAATGTAGGGATTGCAGGGGAGTCTTGGGATGAGGCGAGGGGTGGGGACTGTAAGCCTTTTTTGTAATCCCTCT
GGCTCTTTGCCCCAGCTTGTATGCTAAATCCTCTCCTGTGTACCTCCATTTGTGCAGAACTGTAAGGGCTCAGGGATTCTTCCATTCTGTCCCT
TGATTTCCCCAGCAGCTCTGCTCCACAGTCTTGGTGCTTTCATCTACCTCATACCTATTACCCCTTCCCTCCACTCCCCAGAAAATCAGATAAG
ATAGTGTCATGGGAGATTATATGTAACAGTTCTGTGAACATGTGAGTCCGTCCCTTTCCTGAAGTACCCTACCATGCATATGCCTTTTAGTCTG
TCTTGAGGACAGAGAGACTTCTGGCTCACCACTAATCATCTTTCTTCTTATAGCACCTGAAGTGAGAAAAGGAAGAGAACCAGGACTTAGGAAA
TGCAGAGAGAGAGAGAGAAAACACTCTGTCTATACTAGTAAGCAAGGCATGTTTGCATTTGGTTTCCAGTAGCCAGCAGTGTGGTTCAACCAAA
CAAACTTCAGATGGTTTTTTTTCCACTGAAAACTCTATATCTTCACTGCTGACGCCACTTATTCAGGTTTGGCAAGTCATTGACTCAGCACCAT
GACATCATCCCTTCTTCCTCCCATGCAGAGGAGAGTCAACAACAACCAAACCATGATTCAAACAGCTTGGTTTAATTCTGCATCCTGCCCCTCA
GCTTCATTAATAGGTAGGAGAGTGAGTGGGGAACGGCCTTTAATGCACTTAGTGCCAATCCATTCGGGAAAAAGCATTTGCTCTCCAATCCCGA
GGAGTCTTCTCATATCCCCTAACCAGGCCAGTGAAGCACCCTTGTTTTATCACTTTCCAGAATGCTATGTTTCATCTTCATTACCAAAATTGGC
AAGAAATGTGAAGAGTTTGAGACTGTACCCTACTTGCAAGCTCACAAACTAGCATACCTCAGTTTCACAGATGTTGGCAGAATATATGAGATTC
TTGGGTCAGAGTTTGTTTTGGTTCATGTTTGCATTAATTCCCCTTGCCCTCTGCCCCAAATCACACAGGAGAAATGAGGAGCAGCCAGTCAAAT
GTTGACATACAAGTATTCTTCAAACTATAGTTAGAGACAAAAGCTGTTACAATAAATCTACAAAGGCCATGGACAATTATCTCAACAATATCTC
ACCATTGTTTCTCCACTATCTTGGCTTCTGACAAAGTTTGCCATTAAGTATGTGTGTTGATTTTCTCGATATCAGGGCTGAGCAAGGAAAAAGT
CATGACCATTAGATGGCAGTAGCACACAACATGCTTTATTTGGTTGGCCCTTTGAAACGTTTCAGGAGAGTGGAACTTCCCTCACAGCAGGAGA
CCTTCCAGAGGCAGCAGCAAGAGGCCACCACCCACAACACGAAGACTTCAACGCAACTTCCAAACGAGAGAAGAATTGGACACGAGGCTCATCT
GTCTATGGGATCTTACTCAGCAGCCAGCTGTCGACTCTCTGTGTCAGAGAGTTCCCATGGGCATCAGAGGCTTGGGGCTTTTGTCCAGCCCCAG
AGTATGTCCTATCTACGCCTAGCACATCCTCTGTCTCGTTTTACACAGCGTCCAATGTGGGTGGGTTCTAAAGGGCCAAAAATGTGCTTACTCG
GGGCCATATTTAAAGCAATTGGGTGTGTAAACATTTGATTTTAGTTCCAATCAGCTTTGACCTAATGGTCTTAGCTGTCTCTGAATATAGGGAA
CAAGATACAAGGCACCATCTAAAGATATTAATCCACTTATTTAACAGTCCATGCCTTGGCCCCATGTTGTTTTACAATCTAAGTTGTATGCTAT
TTCATGGAGTCTCAGTTGGGAGCCAGCGATGAAGCATCCTCAAGACCCAGAATGCTATAACGCTACAAATTACGATTTTAAAAGCCAGTTCTCA
GCCAATCTGCCCACTTGGGCAGCCAGTAGAATAAATTTGAAAGGAGACAATTTTGTTTTGACCAGTCCGTTTAGTCAATAGAGTGGCATTTATA
TCCCCTTTGGTACATCACCCCACCTATCTGTAAAACTTGGTGTTATCTACGCACATGCAGGAAGATAATTTTAGCAACCACACAGCCCCTGTGC
CCTGTGACCCCTCCAGCAGCCTGACCTAGGGCTATGCAGTTATCTCACACCATGGCCACTAAAGTACAGTGAGATTCAGCAAAGAGTTTCAGTC
CTTGGGCTGTTTATCTAATTTCTGTGGGTAATTCCTGCAACATGGCTATGTGTTTCTCTTAATTGTGGTGTGCTGTTCCACACATATGCTGTTC
CTACTGCCTCCAAAATTAGTGATCCTAGGGAAGTTAGTACCATGAGGAACTATTTGGAAAGCTCATTTGTGACATTGGGGACTCACATCTAGTG
TGGTTTGGTGGCCGAAAGTACTTGAGGGAGGGGGTTCCACTGTTGAATAAAATTCTACTGGAATTTTGCACCAATCCCAAGTGCAGGAAATATT
CTTCACGGTTGCATGAGGAGAAGATTGTCATAGGGAAGCCATCTGATTCCTTTACAGGACCTCCAGGCTCCCTCTGCTGCCTGTATTCTCTGTG
TTCCACTGAAGGGAAGTTCACATGGCTTTGTGAAGTTCCAGAAGTTCCAGGTTGTGTTAATAGGAGCAGAAAATCGTATACTCAAACAAGCTTG
TGTTATCCACCTTGCATTCAGCACCTGGGTTAACTAAATTTGTCGTGGGCTCAGCCCACCATTGTCTATACATTTATGTGAGATAAGCCTCTGG
ATAAGGTTGTTCAATGTCAATCGTTAGGCGAATCGGCTCATCTAAATAAGTCAGCTATATCATGCAGCGTTGCCAGGAAAGAAGTATTACACTA
TGTTTTCTAMXATCAGGTTGGCAGAAGCAAACCCTGCATTAGCTTAGATTTACCATCTTATATGTTGCTGTGGCTAAGGCTATAGGCACGTTTG
GTGTAGTCGGCAATTGCATGTTCCTCCTTGCTGTGACTGTTGTGTGTTGTGCTTGAGTTTCCTGTTGATTGCCAATAATAAATAGCATTGATGT
GTTCCCATCAGTGTCCGATAGGTTTTGCAGCTGTTATTGGCAGTCCAACAAGAACGTTGTTAGTCTAGCCACACACCAAATTAGATTCTGAATT
ATGAGGCACTCGCATATGAGTGAACAAAACAGGTAAACCAAGTATTTTTTAAAAAAATAGATATCAGCTGGGCACAGTGGCTCATGCCTGAAAT
CCCAGCACTTTGGGAGGCCGAAGTGGGCAGATCACGAGGTCAGGAGTTCCAGACCAGCCTGGCCAATATGGTGAAACCCCGTCTCTACTAAAAA
TACGAAAAAAAAAAAAAGATTACCCGGGTGTGGTGGCATGCTCCTGTAGTCCCAGCTACTTGGGGGCTGAGGCAGGAGAATCACTTCAACCCAG
GACGCAGAGGTTTCAGTGAGCCGAGATCACGTTACTGCACTCCAGCCTGGGCGACAGATCCAGACTCTGTCTCAAAAAAAAAAAAAAAAAAAAA
TATCAATTGTATCAAGGCATAGGTCCCTTTTATGTTCCCTTGTATTGCAACATTATTGTGTTTCAGTGGGGGACCAAAAGCTTTATGGACTCCA
TTTTAGCGGAAACTTTCATAGGGAAGAGGTTTGTATACCTTTAATAACCTCAGGAGTGGCCCCTTGGGAGGTGGCAGTGTGACTACACCAATAA
ATTTCCTCCCAAGTACTCATGGACATAAAGTGTCTACTGAGACACTGGAGACTACTAGAGGGGGACGGGGGAGGGGAGCAAGGGTTGAAAAACA
AACTGTTGGGTACTATGCTCAATACCTGGGTGATGGGATCATTTGTACCCCAAACCTCAGCATCATGCAATATACCCAGGTAACAAACCTGCAC
ATGTGCCCAATGAATCTAAAATAAAAGTTGAAAAAGAAATGACAAGGCATAAAAATGTTTTAATGAAGATGAAAACATTCCTCCCAAGTTAGTT
TCATTACATGTCTTCTCATTATCGTTTATGTCTTCTGTAGGTTTGGAGTGTGTATGGATTTTTAAGGAGCCCTCAGAAAAGTAACATTGGGCAA
CAACTGCCCTCAAATAGTTTCTAAGTTTTTAATAACTGTGGTTTTGGACAAATTTCACAATAGCTATAAGCAAATAGTTATCAATGTGGGTGGA
GAAACTCCCATGCAAACACTAGTTAGATTTCTCCCTGAGATCAAATATGTTTCTTGTAAGAACTGTCTGTGCTGTGACACCACGAGCTATGAGT
TGTTCACTGTGGGTAGCCAGCATTGCAATTGCTGTAGAGTTCACTTCCATTGAGTGTTATGTAAAACTTGCAGTTAGTATATTAAGATTAGGTT
AGGGAAGAATTCCCAAGCTGGTTGTTTTGTGTGTGTGTGTGTGTCTGTGATTTTTTTTTTAACAAATCTCTTGGCCCAGGTAATGTCATGTAGT
ATGCATTGCCTTAGAAGCCAAAAATTGTAAAGGTATACACTGTGGGTGTCCATTGCACTTATTATAATTCCAGGGATAAGAAAAAGCTATCATT
TTATCTATGCAATCCCGTAATGGCTTGTGCGTATTGTATCTGGATTATAGTATTAATAACAACACCAATCACAAGCATCCTGGTTGTAATATTT
GCTGCATACTATCTCTGGGCATTCCTTAAACGAGGCTCAATAGATAGTGGCCCATTGCTCCATTTGTTTAATCGCACATGAGTTAATAATAGTG
GGTATCATCTGATGTGAAGGAAAAGGCGGCCCACAGAGCTTAGAAGGCATCTATGAGTCACAAGTGAAATTGTACAATCTGACTTAGCTGGAAG
GATGTTACTAAGTCGTACTACAGAGAGGAGGAGAAGTGCTAACCTCAGTGCTGATGTCTGTGGACTCACCCAGTGAAATCGGAGCATGGTTCCC
TTTGTTCCCAGTCTGACCTATGTTGGGTGTCCCTGTGGTGTGTATGAGGCATGCTGTTTCCATACATACTCTGCCACCAGTAGGGTGTGGATTA
CCAGGGATAGGGTGATCCACCAATCGAGGTATAATTGACATTGGTGAAGGTAGGAACTTTCTCAGGTAAGGAAAGGGCAGAACAACAGCATAGG
CATAAATCTTCGACGTCATTTGCAGCCAGCAGTAGGGAATCTGATTGTCCTCTGGGAGTCACCCAAATGGTGTTACCAGATACAAGGGTTGTTG
GTTCAATCTCCTGGGTGTCCGGAGCAGGTCAAGGTTTGTTCCATAGTGCCCTTAGTGGAGCAAAGTACATGGGCAGAAAATGAGAATGCTTCCC
TCTCCAGATAGTGGTGGGTGGTTTGCTAGTTTTATGCTCGGTAATGACCAAACTAAGACTGAGAATGCACCCAGAGTGAGTGGTCCATGCCAGA
TCCTTGTCAGGATTGAGAGGAGAAAAAAATAGCCAGGCTCTGGATTTTCTATGAGGCAGAAGGAACAGGTAAAAGAACCAAGTTCCTTTGCTGT
GGGCAGTCACTTCAGGGCCCCCATGTTCGTGAATACTGGTAAATCATGTTGTGTCCAGTGGGTACATACATTACCAGAGCTTGCAGCATCTCTG
TTGGTGGTCTCCTCATTTGCTGTGAATTAAAATATGGTGCATTTATGCCAGAGAGGTATGGATTTAATGTAAGCATGACTATGGGCAGTATTTT
TCGCTACGGGGAATGCTATTGTCCTGCATGTTCTTTCAATATCCCTTGAFTTTAAAAGCCCATTGTGTCATTCTGTGACACCTACTACTGTAGG
ACCAGAGAATAAATGAGAAATCCAAGCAGTGTTCTTTGGTAATGCCTATGGTTGAATCTTCTTTGCAGACAAGTGAGAGTCTTGATCTGACTGG
ATAACTTTGTGTAACTTTTGTACTCCCAAAGGCCATACAAACTATTTTATCAGTTCCTCAATAACATTAGCAGAGCTTGGTGCCTTTCGTGCGT
AAGCCAAGAGTAAGCCTGTGTAGGAATTACTACATTGTTGTGGCATATGTAATAGTTCCTGCTGCTTCAGAGAGGGGCCCAATAAAATTGACTG
GATTTGCCAGCTCTGATATAGCCCTTCTCCTGTGCAGTAGGTCCAAGTCTCTAGATTTCTGGTCTATGGTACCCTCCCTAACAGCTTCATGTAA
ATGTCTCTCTGCTGGGTCTATTTCTCATTAACAGTTGATTTTCATGGTCTCATGATGCCCAAATGCCCTGACCTCTCCTTTATAGGGTCAACGG
TAGCTCTTACCAAAGTTCTGCTGTCTCCCTTCTTTTCAAAAGGATTAACTACTTTGGTACTTTAGCTAGTTTGTCAGTTTCCAGGTTGTATTGT
TTGCTGTGAGCTGAGGTATGGTGTACAAAGATGTGCAGAGTGGAGGCCAATGTGCTGATATTTTCCCCCACATCTCCTTACCCCAAATCTGTCA
GCCTTGTATGTTCCAGCCCTGATTTTGTCCAAGTCCCAACCCAATAGCCTATTAGCCACTGACCAGGAGTTGCCAAAAATGTGAAACTTAGTAA
AATGTTTGTCGAACGCCTTCTCTATTTCTAGGGGTGTTGCACACAGCCTGGCCCATTGGCCACTTTCGCCCATCCCCATATGGAAGTTGGAGTA
TTTGAATAAGGGTAGTACGCTGTTGCTGTCCTTTATCTGACTCCCTTATACCCTGTCCTCACTCCATTGCTAAAGAAAAACATTAATTTTCTTC
TTATGAAAGTGCTGGCCATACTCCCTCCATTCCCCAATGAGCAAGAGCCAGGGGCATTTCCAGGGGTTTTGGCAAGTTAACCTCACTGACAGAG
TATGTGGCCATTTCCTCACACAGTAGTGACACTCCAGGAGGGCCAGGTGTAGTTCAATCCTGTAGATACCATTTTCATTTTAATAAAGAAGCTT
CTGTTGCTGATCCAATTTTATGGGCAGCTCTGTCCATTACCCAGGGCATAACAGGAATCTCGGTCCATAGGGTAATTTTCTCTGCCCCACCAGG
GCTTCAGTTTTGATAAGTGCCCCATATGCTGCCAGTGGGTGTCTGTCTAGTGAATACTTTAGGCTGAGGCTGAGAGCTACTTACACCAAAAGCC
CATGGGTAGGCATTTACCTCTCCATTTCGTCCAGAGGCTCCAGGAAGCAAATTTGGGAGCAGTGGAAACTTATAATTGCAAGTCTTGTCCTTGG
GAGCACTAGAGGGAGAGCCAAAGTATTAGCCTCCTTTAGCTCAACCAGTGACTGTAGATGTTCAGGTCCCTACTGAAAAAAGGAAGCTTTACAG
GTGTCTCGATAGACAATTCAGCAAGGGTCAGCAAATATTGGAGGTGAGGAAGGCATTGTGCTATAATTCCTAATGAATGTTGCGGGTTCTTTAC
TGATGTGAGCGGTTTAAGCATGAGGAGCTTGTCTTTTATCAGATTGGAAATAGTCTGAACACTATTTCTCTCCTGTCAAAGTGTAGAGATCTTA
TTCCTAGATTTAGACTGCCCTTAGGAATCGGCTCAGATAATGGGCCAGCGTTTGTGGCTGACTTGGTACAGAAAACAGCAAAGGTATTGAGGAT
CACATGGAAACTGCATGCTGCCTACCGACCGCAAAGTTCTGGAAAGGTGACCGAGTCTGGATCAAAGACTGAAACATAGCCCCTTTGCATGCAC
GGTGGGAAGGACCCCAGCCCATCATCCTGACCACTCTCACTGCTGTGAAGGTAGAAGGAATCCCGGCCTGGATCCACCTCAGCAGTGTAAAACC
TGCAGCCCCTGAAACTTAGGAGGCAAGACCAAGACCGGATAACTCAGGCCGTGCTCCGCCTGTCAGATAGTAAATCCCAGGTGATGTACCTGCT
GTCAGCAGATCTAGGCTTTCTTCTTGGACACCTTATACCCACAGTCCTCCAGGTGCCAAAGTAGGGCATCCGTTCCCTTGGCAAACCCGACTCC
TGTGGGGTGTCCTAGCAGAAGGTCCTCGACGTACTGGAGCAACACGCACCCTGGGTCTCTGGCAGGAAACTTCTGGAAGTCCCGAACCAATGTT
TCCTCGAAGAGATGGTGGGGGAGTTCTTGAACCTTTGGGGAAGGCGGGTTCAACTGTACTGAGTGGTGACACCCGACCCCAGATCTTCCCACTG
AAAGGCAAACAGTTTCTGGCTCTCAGGGGCTAGTCTGATGCTAAAGAAAGAGTCTTTCAAGTCCAAGCAGGTGAACCAGCTGTCCTCAGCTGAC
AGCAACCCTAACAACATGTACACTTAGGCACTGTTGGATGTAAACTCACTGTAGCTTGATTAACCAAGCACAAGTCCTGTACTGGCCTGTAGTC
CTTGGTCCTTGGCTTGGGAACAGGCAGGAGGAAAGTGTAGTATTCTGAGGTCTCAACAGGCAGGAGTGGAAAAACACCCTTCCGAGTTCCTAGT
GTAGCCTTCAAATTTTACAGACTTATACCTTACATGGTGTAATTGCCAATCCACTCTCAGTGAGAGGTGCTTTACTGAGGGTTGTGATCAACTG
TGAAACAGAGTCTTGGTCACGTTCTGCGAGGAGGATGTCACCGATATAATGCCAAAGCATAGTTTCTGGTCAAATTGAGGGGCAGGTGTCTATC
TCCTGTTGGCAATCACTCTCCAATTGGAGGGCTATTAAGTATCCCCCAAAAAGCTAGCATCACAATGAAGATGAGGGGAAACAAATACCCCAAA
AGCAAGTGGGTAAAGGCGTACTATTGACTCTCAAAGGTCGAAGTGAAATGTGTTTGACTCAACTGTGAAATTTTTACTAAACAGAACATGTTAG
CCAAGTCTACAACTCCAGATTCAAATTCTGGTGGCTTTAGATGCCCTGGATTAATATAAACTTACCACTGATGTTTTGAATGACACCCATTAAC
GCAACAATGTCCTATACAGGTGCTCTAATAAAGGAAACAGCCCTGCTGAACTCTCTATGGTCTATAGTCTCCACTGATTACTAGTGTCTCTTAG
GGCAGGCCATATGGGGAATTGATAGGTGAGGTAGTAGGAGTCAGTGTTCCTTGTTTATCTATGTCACCTGTGACAGGCCATAATCCTTCAGTGT
CCTGCTTCAGCTTCTGCTGTGGTATATGAACCACCTGAACCAGGAGGGGCAGGTTAACTGGTTCCCTCCGCGAAGTGCCCACCAGTAGCATAAA
AACCTTAGTCTTATGTCATGAGTTTATCTGCGGCCTCAGGAGGTCTCTTCCAATAATGGGGAAATGGATAATCTTGGAGGGGAGGCAAGCAGGA
CAGAAGCATATTTTTAGCAAAACTGAGCCAATTTTAATAGTTAAGAGGGGAATTATCCCTTTAATTAATCCCTTTAATTAACCCCCTTCCAAGC
AGGTAGCCTACTTGCCAGGAGGGGGTTCCTTTTAAAGCATGTGTGTGTGTTGGCATTAGGGAGATTTGGGCACCCCTGACTGACAGAGTTAGCC
AGTGTTGTTTGTCAGTGCCCAAGCCACAGTTAATATGGTGAAAGGGCAACTGTCTGTCAGAACCGGCCTTCCGACACTGCAGGATCCCTGTGTG
ACAGAAGATTCTTACCCATTTGGCAAGCATCAGAGACAGGTGTGGAAGATAAGGTTACCTCCAAAGCGAAAGCCAGCTGGGCTGGGAGGGGGTC
GTAATCTAACCCTAGTAAGTATTGCAATTGCTCCAAACATTCACCAGAGATCATCCAATTTGGGACCTTCTTGTGCCTTAGGGTCTAACATAAC
CTCTTTTCGTCATCTCGTGGTTGATGCATACATCGGATCCTGGCCACAGGTGGTTTGTTTGGCCCAGGAGGGGCAGCCTGACTGGTGATAGTAA
CTTTGATTTAGGCCAGGTAGGGTATTATACTGTTACCACGCAGCACCTGACTCTGAGTTTATCTTTCATCAGTTTCTATTAAATCCAGTGCAGC
TATGGTCTCTTGAATTGTTTTAGCAGTAGCCACCATGCCCAGTAAAGAAGATTTTCCCAGGAGCCCCGCCCCCCGAGGAACAATTGAGTGGACT
CAGGACTCAATTCAGACTCCTCCAGTCTTGTCAAGTCATCCTGCCCCACCTCATTTCTTGCCTCCACTCCAACATGTCCAACTGGTGGAGGAGA
TGTTTGGCCTTGGCTAACCTTTTCCCTGGACAGTCTTCCCAGGCCATTATGAGGTCTAATGGTGGGCAGTCTTCTAATCCACAAAAATACACAT
CAGCAATAAAGATGTGAACAGAGGTCCAATGTGGCTTCTTCCCAAATTCTGCTCTAATTATACAGGTTCCTTTAGTGGGGACACAATTTTTCCA
TCATGTACTATCGCTCCTAATTGTTTAATTTCATTCCCCATTAGGTGGAATCAGTTCCCTGCCAGATGCATTCTGTAGAGCCAAGCAGCCCCTT
GTTTATTTGGTTCTTGGCCTGGGTACTTAAGGAGAGACTGGCGCTCGGGTAAGACAGAGGATAGGACTCCTCCTCTGAGCAGGAACAGACCCTT
CCTTCTTGGCTCTCCCTTTTTTTCTTTTCCATTTTTATTACTTTCTGGGTGGCTACAGGCAAAGCAGAGGGTAAGTTTTCCCAGTCCCTACCGT
GCTGACACTGTGCTGCCTTTTCTTCTGGAGAGCACCTATTCTCCTCTCCAGGGACCTCTTGTCCAAACTGTCTCTCCCAAGAGTTTGGGCTCTT
ACCTCTTCTCCAGGTGCCATCCTCGTGATAAATTACTGCCAGCAAGGTCAGCACATGCCTCATGGTTAGGAATCCTTCTTCGACCTGACAGCAA
CCTCCTCTAGCGTTCCCGGAGGGGTAGGAATATAAATTTCTTTGCCTTCAGTCACTTTAAGTACCACTCTAGCTAGCTCAGTCCAAAGGTTGAG
ATATTAATATCCCTCTGGAGAGGGGATAGCATGATGACAACTGCAACATCATAATTGAGTGTAACAACAGTAGAGGCATGCTACCTGCTTGCAA
ACGCCTTTCCACCAGCTGCCCTTAGGAGACCACCCTTGGCCTGATGTCTCTGCCTTATTGGCTCATTTACTTGCCTATCTGTCTCCGGGGAGGA
GCAAATCCTCACATCTCAGACTACCCCAAATCCTCAACCCAGCCACAGGGAGCACTTCCCACCATAGCCTGTTATGCAGTCCAGGGTGGTTCAC
CCTTATTCCAGAGGCATGCACTCCCTCTCCTTTTCTATTCACGTACACAGGTGCACAATCTCCAATGTCAACTGAGGGGGGGACCCAGGTAAGT
GCCCTGACCTCCTTCCCAAGTACCTTGGCATCTAACACCCCAAAAGTCAGTTCTAGTGGTGTCGGTTCACTCAACTACTGGTGCTACCTCTGGG
CCAGCTCAATGAGGTATAGAAGGAGACTGCAAAAAGTATATTGTTGCGGTACAGCAATGAGCGATGCTAAGCAAAACTGCTACAGAGGCATTCT
AAATGTAGAGTCTGAGCACTTAAGGCTGCCTAGGACTGATTTTTCAATTGATTGTCCTACTGACTGGGTAGACGCTATGCCAATTTCTTGTTCA
CGAAAATCCCATCCTCCTCACCACTTGTGGTGAGTTCCAAGATTTCTCGGCTGATCAATGAAGAAGACATGACCACGAGGTGGCAGTAGCGCCC
AAAAACATTTTTTTTTTTTTTTGAGAAGGGAGAAGGAGTCTCACTCTCTTCTCCAAGCTGGAATACAGTCCCCCAACCTCTGCTCACTGCAACC
TCCGCCTCCCGGGCTCAAGTGATTCTCCAGCCTCAGCCTCCGAAGTAGCTGCGATTACAGGCTCCCGCCACAATGGCGGGCTAATTTTTGTATT
TTTTAGTAGAGACGAGGTTTCACCCTATTGGCCAGGCTGGTCTCAAACAGCTGACCTCAGGTGATCCACCCGCCTCCGCCTTCCAAAGTGCTCA
GATTACAGGTGTGAGCCACCGCGCCCAGCCAAGAAGCTTTTTATTTCGGCGGTGCTTTGACAGGTTGCCAGAAAGGGGAGGTCTCTCACATCAG
ACCTTCCAGACCCACCACCAGAGGGCCACTGAGCAGAAGGGGAACAAGGCAAGGTAACTCCTGGGGTAGACGAGAATAGGAGAGGGCCTTATGT
GTCTGGGCGATATGACTCAGCAGCAGGAGAGGGAGTCTCTGGGTCAGAGGGTTCTGGAGGCAAGCACTGGCATGGGGTCTTTTATAGCCCCTGT
GTTTTCTTTAAATGTGGCTACCACATGTCAGTGCAGTTTTGTGGGCCATGCAAGGCAGGCAAGCCCTAAGTGGCTACACATATGCTTACTTGAG
CTATATTTAAAGCAATTGCATATGTGAAAAATTCGAGTTTGGCGTTGGCAGGCTTTCTGCCTGCTGTGAGGAAGTATAATGGCAATATAAAGGC
TAATATATGGAGGTCATCTTTAACCCACTGATATAATAACATGCCACTTACTCACCCACTCTGATTAATCCGACTAGCATGCTCTGAGATAAAA
TCCATTCAATCTGTACTGTCAACAGTACTCTAAGCAGCAAGGTGACATCAATCTCCAACGTTAACCCGAACTTAAAACCTTAAGCGCACACGTA
CAATACTACTGCTCCTGAGTTTTTATCTAAGAGAAATGAAAGCATAGGTCCACATAAAGACTTGTATCTGAATGTTTATAGCAGCTTCAATTGT
GCTAGTGAAAAACTGGAAACAATCCAAATTACCATACAATGAAATACTATACTATGACAAATAGTAATGAACTATTGATAGAAATAACAGCATG
GCTGAATCTGAAAATAACTACGCTGATTTTAAGAAGACAAAAAAGAGTATACAGTGTATAATTCTATTTATATAAAATTCTATAAAGCACAAAC
TAATCTATAGTGAGAGAAAGTAGATCAGTGGTTGCCTAGGGACAGCGATGGGATGGGGATGGGTAGCCAGGGAAGCAACATAAACGGGCATGAG
AAAGCCTTAGGAGGTGATGAAAATGATCGTCATTTTGAATGTCGTAAGGGCTTATGATTATTGCTAAGGAGTTATGCTTATAAAATATAAATAA
TTTGTATTTTATAAATTATATAAATTACATATAATTTCATTTAGTTTTAAATTTAAGGAATGGCAATATGTCTTGCATCAAAGAACCCTCAAAA
CAACACCTTAAGTACTGCAAAGTCTTTGACGATGTATTTGCCAGTTAACTTATCTTTAAACATCAATAATAAATAAAAATGGTCAAGACTTATT
TGTTTCTATGTGTTTATTTTAGTGTGCTATTGCTTTTTCACATTCTGGGTCATATTTTGAAAGTATTATTTCACATTGTGTAAATGTATTTTTT
TTTCACTATTAGTAAAGAGCATCTCAACTAAGGTCCTACTTTCTACTTTGATGGTATAGGAGCATGTTTGACTCAGATATGAACCACTTGTGGA
CAGAGTTATGGGTGAGAAATCTGATATTTAAGGAACACTCTGGATTAGTTTCCACTGGTGAGAAAATAAAAAAGTGATGAGCACTTTTTCCAAA
TACCACGTGATGAATCAAGTACTCAAACATGCTAACTCCATTTAGACTATTCTATTACCAAAATATTCCAATCAATGCCTGTGGACAGTTTGTA
AACTGTCATTCCCAATGCCTCCTCATTCTTCAAGTTCCCACTGAAAGGTCTCATAGCTTATCAGACCTTTGGGGGAAGTGCTTTTTTTTTTTAA
TTAAAAGTTAACTCATGTGCAGGGCAGAGTGAGGCAAAATGCAGCCAGTGGTTAGGACCTAAAGACCTCTCCTTCACCTGGCCCTTGAGAGTTT
GAGAGAAGCTCAGGAAGAAAAGCCAACCATCTGACTCCAGTGGGCTTTGAAGCACTGCTCTTGCATCTGTACATGTGGCATAAATGATGTGAAA
ACCATTAACTCTGCTGCTGATCCAGCTGCAGCGAAAAGCATTTCCTCTGATCAACGTCAACATCTGGCTGGTGGACCTCATTTTGCACCAGGTC
CTGATCGATGACCTGAGCCTGAGTCCACAGTTCACCTGATGGAGAACTCAGAGTCAAATCCCAGCCCCAAGTGAGGCTCTCTGACTGCTGCTTG
AGGGCCTAGGGACCACACCCCTGGAGTCAGGAGGGCAGAGCTGAGGATGACAGTGGAATGGCCACAAGGTAAAGATACATGTCAATATTCTATC
TCCATTAGGTCACATGGATGAAAGGCAAGAGAAAAGTAGCATATACATTTTTGAGAAGTTAAAGCTGATACGACACAAAGATGGTTGATTGTTC
TACAGACGTGGAAGGCAGGGGCTTAGGTCCTGTAAAGTCCCCCACTGAGCAAATAACTGAGACAGTACTTCCTAGTGATGCATTCTGCTGTCAA
ACAGTGTGAAATTTTCCTGGAGAAGGCTCAGGCCATCAGTCCCTGGTCCATCTTCTGGCCCCACAGACATGCTGATCGAACACCCTCCACAGGG
AGAGCATTGCACAAGTGATTCCCTTTTTAGAAATTCTTCTAAGCCAGTCAGAAGAAGACAAGCTTGCAGATGGAAGGGAATCAGAACCTGGGCA
ATATCTTGTAGTGGCCGAGCTATCCCTGTGTGTGGTTTGGAGTGTTTTCACTAAGACAAAACAGTGGCAGAAAGAAAAACCCTACACCCATACA
AACTTTGAGGGAATACTGGAGCTTATTGTAACAGACACTGGTCATGCTATGAAATTTTATTACCTTTTCTGGAGGGGTCGCAGGGCTTTGTGTT
TGGGCTGCTTTCCGTGGTCCACTAATTGTCAAGCTTGCCAAAAGTGTGACAGAATCACGGAATTACCCTTGGCAGAAACGCAGAATTACTCCTA
ACCTCTACTCTCATCCTCCACCTGGACCAGACAGAGAAACAACTTCTTGAGTTTTCAGAAACTACCAAATCTTGGCTCTAGCTCCGCATCCTTG
GCCAGACCATGGAAGCTTGGGGTGAGGGACACGGTGAACAAAAACTGCATCCTCGAGCTGCTCGCACTTCCTCTTTCCCTCTTTCGTCTTGTCT
GTGGGGTCAGAGGTTACCAGGCAGGCGTCACTAAGGTTGTCAGTGGGAGCATGTGAGCCCTAGTGGGTTCCTCCCAGGCAGCTCAACATGGGAG
CCTCCCAATTCCCCTTTCTCCTGCAAGGAAAGATACCACAGCTGTGTCATTCTCCTTCAGAGACTGCAGATTGGAGGTCTGTTTTTCCACAAAA
CCTAGTTCTAAGACATTTCTAGCAAGTGAGACTGACTCATTGGAAGTCTAGTGGTGACAGCTACCCATGGGGCAGGCTTAAATAGCGACTCCTC
CTTGTAGTGAACGAATTTCAGATTTCACACAAGAAGGGCAAAACCATGGGCTATTTTCTCCCATGAATTTTTGCCTATTTTGGGCCCTAAATCA
AATTGAGAAAAGTGTGACTAAAACACTCAAACTGCCAATATAGTTAGAGATATACACTTTGTGAGACTAACTCGGGAATCTACTGCCCTGCTTC
TGCAAGGATAAGGCTCTGTAAGGCATGACTTGAAATGAAATCCAAGCACCTTTTATCTGTTTAACTCTGGTGCAGGAGAACAGAGCAGCATTTA
AGGTATAAATTAAATTAGTCTATTCCTGAAGAGAACATTTGCTACTCACATTTATGGTGGAAGATGCAATCTCACAACACAACAAAGCTCTCCT
TGTCAGTGGAGCAGGCCAATACTGGGTAGGGCTAGACTATAGGGCTCACAGGCTCTATGCCCTAAGCTACTGATGCCCCTTTGTGCCAGAGGCT
GGGTTTTGCAACAAACTCAGTTCAGGTCTGGGAACACTGGGCCCACCGAGAATCCTAAATAGTCAACTAAAATCCCTGAAATACCCAGGCCTGT
TTCTTTAATCATTAGTATTGTTTGCATACCTTTAGGAGGTACAAGCGCAGTTTTGTTACATGGTTATATTACCCACTGGTGAAGTGTAGGCTTA
CCAAAGCAAAAACAGGGTGGTAGATAATCTCCCTTGTGCAATCAACAACTATAACCCTGGCAGGTAGGCTGCCTACTATTCATACTGACTACAG
AGACCCTGGACTCCTCTAACCTGATAAAGGATATGAAGCCAGTTATCTATTTATTACCTCTTAGCTCCAAATTCACCAGTCAATATATATTCTT
CGATAATAGAGGGATTCTTTAAGCATCTCTCCTTTAAAGTAGCATCAAACAGCACGATGTTACACTTACTTAATAGAGGGCTCTGGAGGGACAG
ACATTGAAAAAGGAAGGGTACTTCTATGATTTCTGCCTCTCAGAGGTGTGGAGCTAAGCACATTCAGGATGCTTTCTCCCAGGCCTGGGCCCAG
AACAACTGTCCCTCAGTAAACCTGCAGTCCTGGTGTGCCCTGATGATCAGCTTCTTGTGGCCCTCTTGATAGGGACATCCTGTACTCCAAGTCA
TCTGCCCACTGCTTCCCTCTGCTCACTTGCTCCGCAGCCACACACACACACACACACACACACACACACACACACACACGACCAGGTTCATGTA
GTCCTTTCAATACTGCAGCCCTCTCAACACCGAGCCCCCACCCTGATCCTCTGCAAGGGGCATTGTTTACTCCAGCCCTCCAACTGAGACAGGA
CTCCCAACCCCTACTTCACACCACATACTAGGCACCAGCTATGGCTTGTCCACACTACCATGCTCCAAAGGGTGCCTTCTTGCTTCCTCTGCGA
CCGTAGACCAGTTCTGCCTCTGTACACCACCTTAGGTACTGGCAAACCAGTGGAACTTCTCTGCCATCCACTACGTTCTTCTCCAATTAGGTAT
AAAATCCATCCTTGGGGAGAAGCTTTCATTCTAAGCTTGTCTTTCCTTGGGTACTCTCTCCCAGCCCCACAAAACCCCACATAGAGTTGTCTTG
TATCCTATACAAGATCTTTTATCATCGCTTAATGATTCTTTATATTAAATTTTCCCCTTCTAGATCACTGTGTGCTTTCTATCTCCTGATTGGA
TCCATATTGCTGCAGAACACAACATCTACAGCAAGCATCATTCTTAATGGAGAATCACTGAAAGCTTTCTCTTCAAGAGCTACATCAAGTAAGG
ATATCCACTATCACCACTTCTATGCAATAGTTTACTGGCGGTAGGGGCGGGGTCCCAGGCAGACCAGTAGGGCAAGAAAAACCAATAAGATATT
TGGAAAGGAAGAAGAAAGACTGGAACTATTTTCAGATGATATGACAGTGTATATAAAATTCTACAAAGTATTACAGACAAATTATTAGAATCGA
TAGAAGAGTTTATATGCTTATAGGATAAAATCTTAACGAACAAAAGCCAATTGTATGCTATTTAGAGCTATAAATTTAGACAATAAAAATTTCA
AACATATGATTTGCAAAATATTAAATGCCTAGGATAAAAAAGTTGCCTTGTGGGAAAAATATAAAATAATATTTGGGCAAATTAAAGGTGATCT
TATTAACAGAAGAGATAGACCATAACCACGAATCGAACATTCAATATTGAGAAGTCAAATCTAAAATATCAGCACAATTCAAATTAGAACCCCA
AACTAGCTGTTGGTTGAAGTTTTTACGATTAGTTTATAATTTAAATGGAATTGAAATGGACCAAAAGTAGCCAAGACACTTTTGAAGAGCAAGG
TAGAATTCATTTGCCTAATCCAAATATCAGAATTGACTATGGATCTGTAGTAATTAAGACAGTGTGGTATTTCAAACAGACAAGTGCTTAATAC
AGAGTATAGAACTAGCCCACATATGTGTAGATACTTCATTTATGGCACTGTAAATCAACTGGCAAAAGGCAGACTTTTCAATAAGTGGTTCACT
GAGAAAACTGGATATTCACTTACTTACATACATATATACATAAATGGAACTCTACTTAACACAATACACAAACATCAATTGCAGGTGGATTAAG
ACCTGACTATGAAGGACAATAGCAGACATATTTTAGAAGAAAATATAGAGAATGCTTTCATGGTCCAGGGTTCCTTAAAACAGAAAAGATTTCT
TAAAACAAAAAAGCACTAACCATCAGGAAAAGAATGATGAAATTGACTATATTATAATTAATAGCTTCTGTTCACAAACCGGAGCACAAACCAA
GTGAATATACAAACAGAGCAAGGGAGTATTTTGGCCGCAGGTGTGGAAGAGTATTTAAAATATTTAACAGACTCATTAAGTCAGTATCAGATGA
ACAATAAAATAGAAACGTGGGTTAAAAGAGGTAAATTTGAATAGGCCTTCCTCAAAATAGAAACTCCAAAAGGCTCATTAACACAAAAATGTGC
TAAAGCTTAATAGTAATTTGGATATTGCCCACCAGTAGACCGGCAAAATTTAAATTCGGGCAATACTTGGTGTAGGTGAACATGTGCATCAGTA
AGGACTCTCTATGCTGCTGGTGGGATTGACAACTCTCCCAATCACTACAGAGAACAATTTGGCATCATCTAGTAAACCTGAGGATATCCATTCC
CTATGGCCCTGTGATTCTATTCTTGGATATATACCCTAGAGAGATTTTTACACGTGTCCCATTAGAATGTGCAAGAATGTTCACAATAAATCAT
TGAAAAGTAGAAGCAATCCTAATCTCCATCTGCTGTAGAATGGAGTGGAATAATCATACAATAATCACTTTGCTATAAGCATATGCTGGAAAGA
CAGTCGATGGAGACAAAGGATCTACTTACTTATAGCTATTATGAACAACATGGACGAGTCTCTCAAGTATAATACAGAGTGAAAAAAGCAAGCT
ATAAAAGGAAGCATGCCGTATGATTCCACTTATACAACTTCATATCAACTCTATTGTTTAGGGATGCGTACAAAAGTGATAAAGCTATAAAGGA
AAGCAGTGAAATGATTATTACAAAGTCGCAGCAATTACTATATCTATATAGGAGAGAGTGGTTTGTGACTGGAAGCAGTACAGCAGCTGTATTC
TGGGAGCTTGGCAGTGGTCTATTGCTTGACCTTGGTCCTGGTTAAGTAGGTGTCTGCTTTTGTAAAATAATCTAAACTATACAGATACGTTATA
TGCACTCTTCTAATTTCAGATTTGAAAAGTGCTTCCTCCAGAAAAAGTAATAGAAAGAAAAGTATGTGGAGTAGAAATTTTGATGAGGTTCGAA
TAGTTATGGTTGAAAAACTTGATTTTCAAGAGACCAAGTGTCCACTGTTATTAAAAGAGGGCGAGAGAGCTGGGAGTATTTAAACAACAGGTTA
CACCCCCCCACCCCCACCACCACCAATCTGTTCATCACTCTAAGCCTTCTCATCTCACATATTTCACGCTATATTTGTGTCAATTCCAAACTCA
TTCGTGACAGACAAATTGGAGGATTGTCGTCATCATTAATAAGGACATTAGGCACCAAAGGCCCAGCAAGACTGGGCCACCACACTTAAATGTG
AGCAGGTACACCGGGGCGAGTGGACACAATCGTCTGTGCACATTACTTGGCAGAGTCCATTGAGTGACTGAGGGTTTAACCTTAACACATTTAA
CTGGAACTCCTGCCCAGAGAATGTGCTCGAGAATGCAGAATAAACTGAGCAATGCCCTCATTGTGCTATGAGAGTGGACATGAAGTTTATCATG
GTACAACCGATCGTCGAACGGCAAAGAGTTGGTTTACGTTGTCCACTGTATAGGGAGAAGCCACGGGCGGGTTGGGTTTTTTTCCACATTTCAG
TATAAATTGTTTCATCCTCCACACTTTGCATTTCTACGCTGAAGAGTGCTAATCTGTTTTGGCCGCATCTCTCACTCATTCTAATCTCTGTTAT
CAGCAGACAAAAATTGAACTATGTGGCAAATTGTACCGAGCACTTTGCTCAAGACAATTGTGAGTATGGAAGTTGTCCCGCGCAGACGTGGGAA
GGGGAAGGGCAATTTCCGCAGAAATGCCAGCACTATGGTGGTTTAATGCTGACACAGAGACAGTTTTTTCTCTCAGGAATTGTCTTTCATCCAT
CAATCCAGCCAGGTAGCCAGCCAATAAACAAATTTTAACACACAAAAAGTATGAAGCAACATGTCTAACACTGGGAGGGGCTGGCGTGTGTTTA
TTTGGGTATATACAATCAGTCACGGATTAAAATTGCTTTTACCACATAGATACCTAGGTGTTCCACAGCATTATTGAACAAGATGTCGTCATTT
CCCCATTACTTCACAACACCTCCTTAATTACTTATATTAATTGTAGAATATATTTATGCATACCAGGCTCTACCTCTGGGCTTTCTATTCCCTT
CCAGAGACCCAGTACCAGAATATTTAAATCACTGTGGCGTTGAATACATTTTAATATCTGATAGGGCCAGTTTCTGCGCATTGCACATTTTTTT
TTCCCCTTTCAGGAACGTGTTCACGCCCGCGGCAGGGGGCGGGATCGCGCCTCCTCCTCGGCTCTGGTTCCAGCCGAGCCTCTCGGACGCAGAG
ATGGAAATCCCGAAGCTGCTCCCGGCTCGCGGGACACTACAGGGCGGCGGCGGCGGCGGTATCCCCGCGCGTCGCGCCCGAGTCCACCGAGGCC
CTGACTCGCCGGCTGGCCACGTCCCCACGCGCCGCCTCCTGCTGCCCCGCGGCCCCCAAGATGGCGGGCGCGCGCCGCGGCGCGAGGAGGCCAG
CACGGCATCACGCGGCCCTGGCCCAAGCCTGTTCGCGCCGAGGCCCCATCAACCTAGCGGCGCCGGCGACGACTTCTTCCTGGTGCTGCTTGAC
CCGGTGGGTGGCGACGTGGAGACCGCGGGCTCCGGTCAGCCCGCAGGGCCTGTGTTGAGGGAGGAGGCCAAGGCGGGCCCGGGGCTCCAGGGGG
ACGAGAGCGGCGCGAACCCCGCCGGCTGCTCTGCGCAGGGCCCCCACTGCCTGTCCGCGGTTCCCACTCCGGCCCCGATCTCCGCCCCCGGCCC
CGCCGCGGCCTTCGCCGGCACAGTCACTATCCACAACCAGCACCTGCTGTTGCGCTTTGAGAACGGCGTCCTCACCCTGGCCACCCCCCCACCA
CACGCCTGGGAGCCAGGGGCCGCTCCTGCCCAGCAGCCCAGGTGTCTGATCGCGCCCCAAGCTGGGTTCCCGCAAGCCGCGCACCCGGGTGACT
GCCCAGAGCTGCCGTCCGACCTCCTGCTAGCCGACCCCGCAGAACCCGCGCCTGCTCCGGCGCCCCAGGAGGACGCGGAGGCCCTGGCCGCCGC
CCTGGGCCCCCGCGGACTGCTGGGCTCTGGTCCAGGCGTGGTGCTGTACCTGTGCCCCGAGGCGCTGTGCGGGCAAACCTTCGCCAAGAAGCAC
CAGCTGAAGATGCACCTGCTGACGCACAGCAGCAGCCAGGCCCAGAGGCCCTTCAAATGCCCCCTGGGTGGCTGCGGCTGGACCTTCACCACCT
CTTACAAGCTCAAGAGGCACCTGCAGTCGCACGATAAACTGCGGCCCTTCGCCTGCCCTGCGGAGGGCTGTGGCAAGAGCTTCACCACCGTGTA
CAACCTCAAGGCGCACATGAAGGGCCATGAGCAGGAGAACTCGTTCAAATGTGAGGTGTGCGAGGAGAGCTTCCCCACGCAGGCCAAACTCGGC
GCCCACCAGCGCAGCCACTTCGAACCCGAGAGGCCTTACCAGTGCCCGTTTTCTGGCTGCAAGAAGACATTTATCACACTGAGTCCTCTGTTTT
CCCATAACCGCGCCCATTTCAGGGAACAGGAACTGTTTTCCTGCTCTTTCCCTGGCTGCAGCAAGCAATATGACAAGGCTTGTAGGCTGAAAAT
TCACCTGCGGAGTCACACCGGCGAGAGACCTTTCCTTTGTGACTTTGATGGCTGTGGCTGGAACTTCACCAGCATGTGCAAACTCTTAAGGCAC
AAAAGGAAGCACGACGATGACCGGAGGTTCATGTGCCCTGTGGAAGGCTGTGGCAAATCTTTCACGAGGGCCGAACATCTGAAAGGCCACAGCA
TTACCCACCTGGGCACAAAGCCTTTCGTGTGTCCTGTGGCAGGCTGCTGTGCCAGGTTCTCTGCTCGCAGTAGCCTCTACATTCACTCCAAGAA
ACACCTGCAGGATGTGGACACTTGGAAAAGCCGTTGCCCCATCTCCTCTTGTAATAAACTCTTCACATCCAAGCACAGCATGAAGACGCACATG
GTTAAAAGGCATAAGGTGGGCCACGATCTCTTAGCTCAGCTAGAAGCAGCAAATTCTCTCACACCCACCAGTGAACTTACCAGCCAGAGACAGA
ATGATCTCAGTGATGCAGAGATAGTGTCTCTCTTCTCTGATGTACCTGACAGTACTTCTGCTGCATTGCTGGACACAGCATTGGTGAACTCTGG
AATCTTGACTATTCATGTGGCTTCTGTGACCTCCACTCTGGCAGGGCACCTCCCTGCTAATAATAATAATTCCGTAGGGCAGCCTCTGCACCCT
CCGTCCTTGATGGCCACCAGCGACCCTCCTCAAAGTCTGGATACCTCTCTCTTTTTTGGAACGGCGGCCACTGGTTTTCAGCAGAGCTCCTTAA
ATATGGATGAGGTCTCAAGTGTAAGTGTGGGGCCATTCGCATCTCTGGACTCTTTGCCCATCAAAAACTCCAGTCCAGACCCTCACGCTTTGAC
ACCCAGCAGTAAGCTAACAGTGGACACAGATACTCTGACTCCTTCGACCACCCTTTGTGAAAACAGTCTCTCACAACTACTGACACCAACCAAA
GCGGAGTGGAACGTACATCCTAACTCTGACTTCTTTGGACAGGAGGGAGAAACCCAGTTTGGATTCCCCAATGCAGCAGGAAACCATGGTTCTC
AGAAAGAAAGAAATCTTATCACTGTGACTGGCAGCTCATTTTTGGTATGAAGCAACTCTATTCATTCCTTGCCATGTGGCTAACTTTTATTACA
GTCAATTTTGAGGATATTCTGGACTAAATATTTAAGTGCAGTCATTTCTTTTTGGTTTGCAAAAAGAGCACAGCCCTGGACTATGAGTTTGGAG
ATCTAAATTCTGATCTTGAGTCTGGAACTGACAAGTTGTGTGACCCTCAGCAAGTCACTTAACCTATCTGAGCCTTAATTTCCTTATTTATAAA
TTGTSGTGGTTTCAACACATTGCTCATAACGTCTTTTCAGTTTTGTTTTCTTTTGTTTTGTGATTTTGTGCTTTTTCTTGAAAATTTTCGGGCA
TTTTGCAATTATTATTGTTTGTACTTGTAATCAGAAGCGGTTTGAGCCCTGTAGCACTAAATAATGAAAGATTGAGGAACTGGTGTTTTACATT
AAAAATTTAATGGAAATTTTACACAGTACGAAAATCTAATCATACAGCTCAAAAAAAAASACCTAAAAATAGGAGTTGGTTCTTCTTAGCTGTT
TATCCTTCTAACCTTTTTTTTAAATGATGAAGGTATGTTTTTGTTGTGGAAAATACAACGGCTTTTGTTATCACTCACACCCAGAGTGAAATGT
TTGCTTTGTCGTTTTGAAGAAGTTGGCCTTTAATAAGTTATTTAGTCTCTCTTAGCCTCAGTTTTCTATCTGTAAAATGGTCATAGCAATGTGA
ACATATGTGTTACATCCTAGACTTATTTTTCTACCCCAGTAGGTTGTATTGAAAGCAAAATGTTATATGTGTTTCAGCATGTTTTGGTGAATCT
TCATTCCCTTTCCTGCCCCTTGTTTTCCCTCCTCAAAGGGGAGAAATTAGCACAAATTAGTATCAGGATTGTGCAGGAAATAAACATTTGTGAA
CCTTAAGAAAGAGGAAAAGGAAGTTATTTCTTCAACAGATTAAGGATTTTTCTACACACAGTTCTTTTGTGGCATTGGCCACATCTCCATTAGA
CCAATTTGATAGTATCTTCGGTCTGCATTCAAAGCCAGCTCATGCAATGAGTATTCAGCCTATTCTTCCAAGACAATCTGGACATAGACCAGAG
CGACATTTTTCTCCCCTCTGTGCCCTAGAGAGCTCATTGCTGGCTTACTCTTAGASTTCAAATGAGAGGGTTTTG
HUMAN SEQUENCE—mRNA
CTGCTCGCGGCCGCCACCGCCGGGCCCCGGCCGTCCCTGGCTCCCCTCCTGCCTCGAGAAGGGCAGGGCTTCTCAGAGGCTTGGCGGGAAAAAA
GAACGGACCGAGGGATCGCCCTGAGTATAAAAGCCGCTTTTCCGGGCTTTATCTAACTCGCTGTAGTAATTCCACCGAGAGGCAGAGGGAGCCA
CCGGGCGGCCGCCTAGGGTGGAAGAGCCGGGCGAGCAGAGCTCCGCTGCGGGCGTCCTGGGAAGGGAGATCCGGACCGAATAGGGGGCTTCGCC
TCTGGCCCAGCCCTCCCGCTTGATCCCCCAGGCCAGCGGTCCGCAACCCTTGCCGCATCCACGAAACTTTGCCCATAGGAGCGGGCCGGGACTT
TGCACTGGAACTTACAACACCCGACCAAGGACCCGACTCTCCCGACCCGGGGAGCCTATTCTCCCCATTTGCGGACACTTCCCCGCCGCTGCCA
GGACCCGCTTCTCTGAAAGGCTCTCCTTGCAGCTGCTTAGACGCTGGATTTTTTTCGGGTAGTGGAAAACCAGCAGCCTCCCGCGACGATGCCC
CTCAACGTTAGCTTCACCAACAGCAACTATCACCTCGACTACGACTCCGTGCAGCCGTATTTCTACTGCGACGAGGACGACTACTTCTACCAGC
AGCACCACCAGAGCGACCTGCAGCCCCCGCCGCCCAGCGAGCATATCTGGAAGAAATTCGAGCTGCTGCCCACCCCGCCCCTGTCCCCTAGCCC
CCGCTCCGGGCTCTGCTCGCCCTCCTACGTTGCGGTCACACCCTTCTCCCTTCGGGGAGACAACGACGGCGGTGGCGGGAGCTTCTCCACGGCC
GACCAGCTGGAGATGGTGACCGAGCTGCTGGGAGGAGACATGGTGAACCAGAGTTTCATCTGCGACCCGGACGACGAGACCTTCATCAAAAACA
TCATCATCCAGGACTGTATGTGGAGCGGCTTCTCGGCCGCCGCCAAGCTCGTCTCAGAGAAGCTGGCCTCCTACCAGGCTGCGCGCAAAGACAG
CGGCAGCCCGAACCCCGCCCGCGGCCACAGCGTCTGCTCCACCTCCAGCTTGTACCTGCAGGATCTGAGCGCCGCCGCCTCACAGTGCATCGAC
CCCTCGGTGGTCTTCCCCTACCCTCTCAACGACAGCAGCTCGCCCAAGTCCTGCGCCTCGCAAGACTCCAGCGCCTTCTCTCCGTCCTCGAATT
CTCTGCTCTCCTCGACGGAGTCCTCCCCGCAGGGCAGCCCCGAGCCCCTGGTGCTCCATGAGGAGACACCGCCCACCACCAGCAGCGACTCTGA
GGACGAACAAGAAGATCAGGAAGAAATCGATGTTGTTTCTGTGGAAAAGAGGCAGGCTCCTGGCAAAAGGTCAGAGTCTGGATCACCTTCTGCT
CGAGGCCACAGCAAACCTCCTCACAGCCCACTGGTCCTCAAGAGGTGCCACGTCTCCACACATCAGCACAACTACGCAGCGCCTCCCTCCACTC
GGAAGGACTATCCTGCTGCCAAGAGGGTCAAGTTGGACAGTGTCAGAGTCCTGAGACAGATCAGCAACAACCGAAAATGCACCAGCCCCAGGTC
CTCGGACACCGAGGAGAATGTCAkGAGGCGAACACACAACGTCTTGGAGCGCCAGAGGAGGAACGAGCTAAAACGGAGCTTTTTTGCCCTGCGT
GACCAGATCCCGGAGTTGGAAAACAATGAAAAGGCCCCCAAGGTAGTTATCCTTAAAAAAGCCACAGCATACATCCTGTCCGTCCAAGCAGAGG
AGCAAAAGCTCATTTCTGAAGAGGACTTGTTGCGGAAACGACGAGAACAGTTGAAACACAAACTTGAACAGCTACGGAACTCTTGTGCGTAAGG
AAAAGTAAGGAAAACGATTCCTTCTAACAGAAATGTCCTGAGCAATCACCTATGAACTTGTTTCAAATGCATGATCAAATGCAACCTCACAACC
TTGGCTGAGTCTTGAGACTGAAAGATTTAGCCATAATGTAAACTGCCTCAAATTGGACTTTGGGCATAAAAGAACTTTTTTATGCTTACCATCT
TTTTTTTTTCTTTAACAGATTTGTTTTTAAGAATTGTTTTTAAAAAATTTTAA
HUMAN SEQUENCE—CODING
ATGCCCCTCAACGTTAGCTTCACCAACAGGAACTATGACCTCGACTACGACTCGGTGCAGCCGTATTTCTACTGCGACGAGGAGGAGAACTTCT
ACCAGCAGCAGCAGCAGAGCGAGCTGCAGCCCCCGGCGCCCAGCGAGGATATCTGGAAGAAATTCGAGCTGCTGCCCACCCCGCCCCTGTCCCC
TAGCCGCCGCTCCGGGCTCTGCTCGCCCTCCTACGTTGCGGTCACACCCTTCTCCCTTCGGGGAGACAACGACGGCGGTGGCGGGAGCTTCTCC
ACGGCCGACCAGCTGGAGATGGTGACCGAGCTGCTGGGAGGAGACATGGTGAACCAGAGTTTCATCTGCGACCCGGACGACGAGACCTTCATCA
AAAACATCATCATCCAGGACTGTATGTGGAGCGGCTTCTCGGCCGCCGCCAAGCTCGTCTCAGAGAAGCTGGCCTCCTACCAGGCTGCGCGCAA
AGACAGCGGCAGCCCGAACCCCGCCCGCGGCCACAGCGTCTGCTCCACCTCCAGCTTGTACCTGCAGGATCTGAGCGCCGCCGCCTCAGAGTGC
ATCGACCCCTCGGTGGTCTTCCCCTACCCTCTCAACGACAGCAGCTCGCCCAAGTCCTGCGCCTCGCAAGACTCCAGCGCCTTCTCTCCGTCCT
CGGATTCTCTGCTCTCCTCGACGGAGTCCTCCCCGCAGGGCAGCCCCGATCCCCTGGTGCTCCATGAGGAGACACCGCCCACCACCAGCAGCGA
CTCTGAGGAGGAACAAGAAGATGAGGAAGAAATCGATGTTGTTTCTGTGGAAAAGAGGCAGGCTCCTGGCAAAAGGTCAGAGTCTGGATCACCT
TCTGCTGGAGGCCACAGCAAACCTCCTCACAGCCCACTGGTCCTCAAGAGGTGCCACGTCTCCACACATCAGCACAACTACGCAGCGCCTCCCT
CCACTCGGAAGGACTATCCTGCTGCCAAGAGGGTCAAGTTGGACAGTGTCAGAGTCCTGAGACACATCAGCAACAACCGAAAATGCACCAGCCC
CAGGTCCTCGGACACCGAGGAGAATGTCAAGAGGCGAACACACAACGTCTTGGAGCGCCACAGGAGCAACGAGCTAAAACGGAGCTTTTTTGCC
CTGCGTGACCAGATCCCGGAGTTGGAAAACAATGAAAAGGCCCCCAAGGTAGTTATCCTTAAAAAAGCCACAGCATACATCCTGTCCGTCCAAG
CAGAGGAGCAAAAGCTCATTTCTGAAGAGGACTTGTTGCGGAAACGACGAGAACAGTTGAAACACAAACTTGAACAGCTACGGAACTCTTGTGC
GTAA

TABLE 2

MOUSE NOMENCLATURE
ICSGNM Bach2
Celera mCG12764
HUMAN NOMENCLATURE
HGNC BACH2
Celera hCC33075
MOUSE SEQUENCE—GENOMIC
GTGCGCTCAAGATAGAGACCAGCTACACAGCAAGTTCCAGGTCGGTGTGGGCATGAAACACTGTCTCCAAAGCCACAGGAGACAAAGGGGGGGG
GAACAACAGGTAAGGGTGACCATGTAAAAATTAAAAACTTCTGTGCTTTTAAAGAAGACCATTAAGAAAGTAAAAAAGACAAACCTTATTCATG
AGATAAAATATGTGCAACTTGCATATCTAACAAAGGTTTAGTATCTAGGCTATATAAGTAATTTTTGTAATTCAACACTCTTAAAGCAAATAAC
TCAATAACAAGCAAAGAATTAAAACATTTTCCAGAAAATATGTACATGAAACGACACTCGACATCATTAAACATTAGAAAATATAGATTACAGT
CATAATGAGATGTCACCTTACCCCCAGTGAGATCAACGTCCGAAAGACTACCACAATGCCTGTGGGGGTGTGGAGACCCTACTGATGGATTGTA
AAAGAGTACTATCATGGGAAGCGTTTTGAGCAGTTGTTCAAAATGTTAAAGAATCAGTGTATATCCCAGTACACTAACCAAGGTTCTCCAGAGA
AATAGAAGGGTGTGTGTGTGTGTGTGTGTGTGTGTGTAAGAAAGACTCGAGAATAAACGAGAATAAATGTGTTTTGAGGAACTGGTTCACATGT
TTATTGTGGAGGGCAATATTATCACTTGGACAGGATCTAGGATCATTTGTGAGACAAGTCTATGGGTCTACCTGTGGGGATTGTATAGATTAGG
TTAGCTTCTGGCCAGGCATGTGAAGGATTATCTTGATAGCTTTAATTGAGGTGGGAAGACTCACCCCAAGTATGGGCGGACACATTCCCTGGGC
TTGGTGTCCTGGACTGCATAAAGGGACAGAGGGGGGGTGGCGGGGGGGCGGGGGGGGGAGTAATCTGCACACCAACAGTCACCATTCTGTTTCC
CCACTTCAAATGCAGTATGGCCAGCTCAAACCTACCATGACAGACTGGATCCTTGAACTGTGAACCAAAATGAACCCTTTGAGCCTTAGGTTGC
TTAAGGGCTCCATGTGTGTGTGTGTTAATTTTTTTCCAAGGAAGGAGTCATCAGGTTGTAAAAGGACCTTTGATAGGAGAAATGTTAAAACATT
ATTTAGAGTGCCAGGCTAATTTTCTGGCCGTGTGATACATTGCACTAAGTGGTAGGAAGATTTTATCACAGCAACAGGAAAAGTGTTGAGAAAA
TTAAGACAATATGGAAGCTAACAAGTCTAAACTCTGTTTAGACTCTAAAGGCAGACAGTCTGCTCTCCCTCAGAAAGGCCTTCAGCCGATTGAA
TGAATCCCCCTTGTATTCTAGACAGTAATTTGCTTTACTTAAAGACTACATTAGTAAATGCTAAGCCCATCTTGCTTCAAGAGCAGAAACCCTA
GTGTGGACTGAATAAAGATTGCCTCACTCAGTGGTCAGAGTGAGCAGATGGCATACTCCCAAGAAGCAACATAGGAAAGACTAAATGATTTTTT
TTCTTTTTAAGAGCAGAAAATGTAAAACTTAAATTCAGAGCCCGGCGTGGTCGCGCACGCCTTTGATCCCAGCACTCGGGAGGCAGAGACAAGT
GGATTTCTGAGTTCGAGGCCTGGTCTACAAAGTGAGTTCCAGGACAGCCAGGACTATACAGAGAAACCCTGTCTCGAAAAATAAAACAAAACCA
AAACAAAAGACCAAAAAACCCTTAAATTCAAAGGGGGGGAGCGTTGTTACTATTGTTTGTTTGTTTGTTGTTTGTTTTTAATTTGGTTTACGTT
TTTCAGTGATGTAAGTGGTGCAAAGAATCAATGTCAGCAAAAAAACAACAACAAAACAACAGGAAGTTAAGTTGGATCTAAGATGACTGTAGCG
CCTACAGAACATCAGCTAGACCAACACAGCAGAGACTGACCAGAGGAGCGGGTGTTCCCAACACTGTCAGCTAACTTGGTGCAGCCATGTTAAC
AATAGGATTGACCAGAAGCCAGGTGGCGATGTCCCTAAAGCCAAACCAGTGCTGAGGCAAACAGCACCAGAGGAAATCACCCCGAGATGCCCGG
GGCCTTAGTGCCAGTGCTGGGGCCTGGGAACAACATGAACTCTAGTTCTTCTACAGATTTTAATCTGGAGTTCACAGATGCATAGAGGACCCCT
AATACACTTGAAAAGGTCCTTGAACCTTTGATTACCACATGGAGCAGTTTTGTGTGTGTTAATTTTTTTCCAAGGAAGGAGTCATCAGGTTGTA
AAAGGACCTTTGATAGGAGAAATGTTAAAACATTATTTAGAGTGCCAGGCTAATTTTCTGGCCGTGTGATACATTGCACTAAGTGGTCTGGCAC
TCCCAAGTCTTGGACTAAAAATGAATTCTTAGTGCATGATCTCACTGCCTGCAGGGCAGTTTGATTCTGCCCTGGCATTTGATGGGACTTGTTA
GAGGATTCGGAAAGGCAGGATGTTGGTACGTTTGAGGCCATTAAACCAAGAAGGAAGAAAAATATATTGTGGAGTTGAATTAAATGCTGAATGG
CTAAGGACATTTTTTATGGTCTTTGTCAACACTCGGAAGGAAACCCTGCTTAGAAACAAGTGAGTGACAAACCAGCTCCGTCTAGATGCAGCAT
GTTGGGTAAACAAGTCGTCTTTCCAAGAAACGCCGCTTCAGTCATGCTTTGATAGTGGAGACCGGGAGGGCTGGGATGCTTGTGTCTGTCATTA
TAACTAGTATATGACCTAGAAAAGCATCCAAAGGTCCTTTTCCATATGGAGACCAGAGTTCCTACAGCTCTGGTGTCAAATGTGGAAAACAAAT
GAAGGGTGTGTCCTGGAGGCCCTGATCTATAGGAGAGATTCTATATAAAATGAGGCACAGCCCTCAAAGTGCCTCTCTCTCCAGGCCCTTGCTT
TAAGTCTTATTTTTGGTCTTGATCATTAATTCCCATTACAGTAGATGGACAGTGGTGATGGTTGCTCAACCAGAAATGTACTTAATGACCACTA
AACTGTTTATCTAAAATGATGAAAATGGCCAATTATAAACTCTGGATACTTTATTATGACAAAAACATAGGATAATACAGAGTGGCGACGGTCC
ACTTATCTTATGTGAGAAGATTTCTGTGCAGGAGAACTAAGCGTTCAGAATCTTGTCAGTGGCCAGTCTCACCCAGAGAGATCCTGAGCGCCCT
GCCAGCCAGGGAAGAAGAGAAAAAGGACAGGCCAATTTCTGTGTCAGTAGGAATAATGAGCCAGGTGAGAAATCACAGATGATAAATAAAGTAG
AGACGAGGATGGGTAGATTGATGTGAGATGTAAGGTGAAGATAGAACTGACAGCCTGTACTGAGGGCCTAGCCAGCAGTGAGGAGCAAAGATGG
TTCGTGGTTTTTTCTGCTGGAGCAGTTGGGTCCACGGTAGCATCTTCTGTTGACATGCTGCCTCATGCCTCTCTTACCTGCCGCTCGCCTCACT
TGTCTTTAAGGGCTCTCCGCATGACTTCCTGTAGCCTTTCCTTGCTTTAGCTGCCATCCTTGTGTGGCCCCACAGGGTCAGCTCAGAAACTTGT
GAGTGTGCGATCCGGATGCTCAACTGTGTTGCCTGGATGGCACGAAGCAAGCCAGTCTGCAGATGAGACTGAGAGGCTGTGTGGCTCTCAAGAA
AGGGTCAGAACTGTGCAGGATCTCTGTTGGCTTCTAGAAGAAAGATAGCAGAGGTCTTAGAAGACTGCAAATCTTAGGAGGAGGCAGAAAGATC
TCTACAACTTGACCAAGTACTGAAGCTAGGAGGAAGAGTTAAGGCATTGTTGATGACCATCCAAGATCCAAGAAGAAACATTCTTCATGAAGTT
GACAGAAGCCCCTAAGAGGTATGCCTGCTTGCAGCCCACAGTGAACTTGATGTCTGGCTGTGTGATATAGTATCCTTGAAGTCACGTGGGCACT
AATACCCCGATGCCATTTGAGAACCGACTCTGCGGATGTTTTCTGGGTGGGCGTTGCCAGGGCTGATGATCCTCTCTAGAGGCTGGATTTCAGG
ATGAGATTGCTATTATGGGTTTTCCCTGTGATACTCAGCAACCATGGACCATATGCTTACAGTTACTCCTCGAGGGAACTCTGACAAGATGATG
ATACTGCTTTTTCAAAAGAAACTACAACAGAAGGGGAAGTGGGGGGACACACCTCTAAGTTGTAATGTGCTTCACAACGCATGAGCACTAACAA
ACTAAGCAGTCGGCTTGTGTACAAACTGCCATGGCATCTGCTGCTTTGCCACCTTGCTTTGCTGTTTTACTGAGTACACAGAAAAAGTGAACAC
CATTTTTTTGAGGAGGGGAACGTACTCTCTGACTGGTAGTGAATATCAGTGTTGTGTAGGAAAGAGGTGGCTGCTTCCTTGGGTGAGCCTGGGT
AGTCCCTTTGTCCAGCGGTGTGCCCTTAGGTAAGGCACTTTGAGTGTCCGAGCCAAGGTTCTTTTATCCTCCAAGTGGGCTAATAACACTGAAA
ATCCCTAGCTCAGTCCCTAATAGAAGTTTTCAGTAAAGGAAAATCACGGTAGTAATACAGACGCTGTACCCACAGACTGGTGTCTGTTAGTTAC
AGAGTCAATGAAAGTCTAGGAATTAGTTCAGATTAGCTAGTTTTCACTCATTTGTTTTTGGTTACTTCTCCTTTTCTTATAACTCTTTTTTCCA
AATCGCTTGGTGACAAATAGTTCAGGTTGCAACCTCTTTTAGGATAGGATAGATAACATCTGAATTTTGCTATATCTGGGAACATAAACACCGT
CCTTGGTTGTTAGACAGAGTGAATTCAGGCTCAGGAAGAGAAGGACCGATGGTCTGAGCACTGTATCCCAGAAACTGCATTCCAGCTACAGTTC
CACAGACAGAACAACTAGGTCCCTGCACCTGAAACATGTAAATGGCATCTCCCTAGGGTATGTGCCTGTTCGTGTGCACCTGTGTGTGTGCTGG
TGGTTGTCTGTGTTGTATGTGTCTCTCTCTGTCTCTGTGTGTGTGTTCAATCTTAATTCACTTTCTCCATCTTATTGAAGTGCCTTTATCTCTT
CCCCCTTTTTGCTCTGTGATAGCACAGGGCACATTTCCTTGTGTTGCTTTCCCAGGGCAGTAGAAGACACAAGAGCCCCAAGATTTGCAACCCA
AACATCTCCAATCCACATCATAAGTGGATGTCTAAATTCTGTCTGAAAGATAATTTTTAAATTCATATTTATAGATTCCAAAATTTAATGCCAA
GTGAACCCATTACTTGTTAAAGCTTTTCTGGCCACAAAATCGTGTCCACATTTAGTCCTTCTTGGTATGAGTAAGGACTTTAGAACCACTGTCA
GTAAATCTATAGCTAATATTTTGGCAGAACATTTGAACTTGATCTTGCTTAGTAGCAAATAATCTCTAAATGTTTTTATTTTTGGAATTGAACT
TTTTGTTTTCCGATCACTGTCCATTCTCTAGGCACTCAATCCAGAAGTGGGGAATGCCCCTGTGTAAATGGGGATTCGTCTCAGTATTAGCCCT
AAAAACCAGTAAGCTGGTTTTGGACAGAAGTGGTAGCCTGAACCTAGAAAACTCACCTTGACCTCTCTGTGCAGCATCTCTGCTGAGAAAACTG
TTGCTGATTTGTAGAGAGCATCCACCACTTCAGGAGATAACATTGATAAAGGAGAGAGAAGGAGAAACTTTTAATCCCACAGCCAAGGGGTGCC
ACCGTGCATGGTTGTTAGCGTGCATTCAGGCTCAGCACAGACATGGACCGATGCTCTGAATACTTTATTTTCTATCCTTTGTGTTACCCCACAT
CCTGCGAAGGTGGGAAGGAGCAATTAGGGAAGCAATTTAATCTCAGGAAGACACCAGAGAATCAGTGCTGGGAGTGAGAGGAGCCCTGAGCTAC
CTCTCTGTCTTGAAGAAGTGATTCTGCTCGTATGAGATCTATGTGTGGGTGAAAATGCCTCCACCTGCCGTCTCCTGGGGGAAGTAGCTCATCA
CATCCTCTTAAGCCCACTAACACCAGCTCAGCTAGGAGTTAAGCTGTCGGCGATAAAGAACGCAATCAGCCTTCCCTATACAACTTTCCAGAAA
ACCCAATCGTTTCGCAGCTAATTGGTACAAATGTTCCCTTGCCCCCTGTTTACAGCTGGAGACCCTCTTGAACATTTTTATGTAATTAAACACC
ATTTACTCTTCCTACCTTAAAAATGCAGGTCCTGCCGACTCTTTCTTTTAAGTAAATTTAAGATTACGCAAGAGGGAATTATAGACAGATAATT
TCCCACATTTCCACTACACTACTTTATTTGGAAATGAATGAATAGAAACCACTCACACGTCTACTTCTGCAGTTCCCCCTCCTTCTCTTAACAG
TAACTATGAAGAAAGGAAAAGGCGGTGTTGTCATGCTTGGTGTAGCTGTACCACAGCACATCACCCACTGTTTCCTGAGAACTGTCCACTGAGC
CACTCTGTGTGTGGAGTCTTTGGAAATCAATCTTGTTAGAAAATCCACTCCAGGCAGGAAGCGTCCAGCACTCCTGTATTCACCGTTGTTTCTA
ACAGGCCAGGAGTTCAGAGCGCTTACTGGCTGAATGGGGGTGATATGTGCTATCCTGTGGGCCACTGGGCCTCGAAGACCTCCAAAAGAGCAGT
GTCCTATGGTTGTGTTGTCCGCTATCCATGTTTAGCTATTTAAATTTCACTTAACTTAAAATCAATTGCTCATTCTTAGCAGGTAGACTCTAGG
TACTGGGTCGCCACACTGAGGCTGCTGTGCTGTCCTCTCCTCTGCTGTGAATGCTGAGGTCCCCATTATCATAGAAACCTAAATTACTGGCCCC
AAGATTTTTAAGAGCATCCCTTGTGACTATAGCATATAGTTAACACAAAAATCCTATTTACCAACCTGCTTGACACTAGTGGTCAAATACCAAT
CAGATTGCTAGCAAGACACCATCCAGCAGTATGAATTTGTTTCACTATACCAGTTGGTGAAATAATCAAGCACCAGAGTTAACAGACGCTCGGT
GCTCTAACCTCTTCACTATCTGCTCAGGCTATTTCCCTAAGGTTCTCCGGCCCTTCTTCAGTCATCCTAAAGTGAGGAAGCCAATGACCTGGAG
GGTCACAACACTGCAGGCCACATTGGTGGCTGGTGCTCTAGGTGTCATGGTGCCTTGACTTTGAGAAGGGGACACTTTCTAAGCGTCATTCTGT
ACGCTCACATACCACTGTCATGGCCATCACCCAAATGACAGAACTATCAGCTTCAACAAGACCTCAGACATCATCATCTCATGACCCAACAGAG
TCACTCAGCTAGTTTGTGACCAACTGAGAAATACAACACTTAGTCTGGTATTTATTTATTCACTCATCTATTGGCTACCTCTTTCTGTGTCTAC
TGTAAAATGGGGATTATTAACCTTAATGCCTGGCAACATTGTTAATAAAAAGCAAGATGTTTCTCAACCTTAAATGTATGTCTTATAATTTGAA
AAATCCCCCCAAAATCCCCACATATTCACATATATTCATATACATATTTTTTCCACCTCATTTAATCCTAAGAAGAACCTGTGTGAAGAGTTAG
GCTCAGAGGCAGAGCACCTGTCCCACATAAGGAAGCCACTGTGACACAGAGGGCTGTGTCTCTCCGGACTTAGGAACAATAATCTCTTGTAAAT
GTACGCCAATACAACCATGTGGCATTGCTAGCTTTGCGCCTGTGGCATTTTGCACTTCAGGAGTTAGCGATGTTCGAAGTTATGGCTTTGCTCG
AGTGGTGCATTTTTTCTTTTTTACACTTTGATAATCTGCCAGTTGCACAGACTTTGGTTCGGCATGGGCGGCTTTGAAGTATATTTTTAAAATG
AGAGAATCCAGATTTGTAATTAGCTTTGCAAATGCTAGTTGTACAGTTTCAGCATCCGAGGAGCTCCTGCTAATGTCCCTGTTCCTAGTGGCAG
CTCTGTGTGATTTTTTTTTTATTCCCTCTCCCTCCCCCAAGCAATTAGGAGAAACAAAGTCTGCTCTCTGAGGATCACTTCCTCACCTCTTCAT
TTGTATAAACTATGAGCTTGGTGACCTCACCCAATTAACGATATACCTGATTGCAACAAAGGACAAACCTTTATAAGCCACTCTACCCTCTTAG
GCTCCTAATTCAGGACNNNANNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNATGTGTATGTATGT
GTCTATGTATATACATACACCATGTACACATCGAATAGCATTTAGTGAAAAAAGAGACCATGAATTTCAGGATTTTGGAGGGGTATCCGGAAGC
CTTTGGAGGAAGGAAAAAGAAGGAAGAAATGTAGTTATAATATCCAAAAATCTTTAAATAATTTTTTAGAGAACAAAAGAAGAACCGTGGTTCT
GCATCCAAAGTACCCAGGTTCAGTTCCCAGCACCTACTTGGAGGCTCCCAACTGTCTATAACTCCAGTCTCAGGGGATCTGACACCATCTTTTG
GCTTACGCAGGCACTATATACAGGAGGGCTGCATGCACATGGTGCATTGTTATACATTCAAACAAACACTCATACACATAAAGTAAAAATATAT
AAAATCTCAAAAAAAAAACCCTAAACAAAAACTTGTGAGGACATGCTTCAAGCTCACATCACCAACGTGGTCGCTTGTTACCTTTCTAGAGACC
CAAGAAATGCTCAGAAACTCAGGCAGCCTGACACTCACGTCACCCTCCGTGTGTCGCTTGCACTGACAATGAAGGAAAACAACTGATAAGTCCT
CATTTGAGAAAAACAATTCAAGTGTTTCTATGAAATATTATAATACTACAGGGATTCAAAACTGTGTTTTCATTAACTGTAGCAACATTGCGAC
ATGTTCTCTCTTATGTAACTCATATTTAATTTCATATTTAATAATTAAATAAAAACCCTCTATGTTTGTGTACCAAAAGAATACTGGTCCACAA
TCCAATTTTAAATCCAACACCTTTCAGGGTGTGGAGTCTGTGTCCGTGTGCGTGCATGTGCACATGTGTTCATGTGCATGCATGTACATGTGTG
TATCCAACCCCAGAAGTTACACTGAATGGGCAGTGAGCTTCAACAACAAAATATCCTTAGTCATTTATTGGTCAAAGTTACCACTGTTCATTAT
TTTTTTTCCTTTACTCTTTACCTGTTTTTATGTGCTCTACAACAAACATTAACTCATTATCAGAGAAGAGGAAATAAATATGTCTAATCTTATG
AATCAAAATTTTATCTGAGGACTTAAGGTTACCTTAAAGCCAACAACAACCTTGTCTGACTTGAAAATAAACACTTTGGACCCATAGATGTCAG
CCAGAATTCACAAGTTAATCTAGAACCTCTTTTACTTAAGACCACCACTTCGCCCTCCATACTGGCGCGTTTGAATTTGATCAAGAACGCCTTC
TGAGATGCAGCCGGAGAGATCGGGGTGACTGGATCATACCCACATCCAAGCTGTGGGGGTTCAAGTGAACACCTGATAAGGGAAGCTTGTCTGC
AGGGAACGCCTGCAGAGGAGGTCTTCTGCCCGAATAGAGCCTACATATTGGTACCTAGGGGCGATTGTCTTACTCTCGCCTTGTAGTTCGTTGA
AGAGAAACACTAGAACCACTGTCGAGTACTGTGTTGTTCTCAAAGTTGAGTAGAGTTTCTGAGGCAGCAGGCAGGACATTCACGGTGTGCAACA
CTCATCCACTAACGAACGGGCTTTCCCTTCCGCTCTAGGTGGACACTCACTGATCTATCACAAGATTGCCTCACTGAGTTTGGGGGTCCATGTC
TGAATGGATTGCTGTAGCCTTCTCATCTCTTCCTCTGCCCAGTTCCCAGCATCCTAAGACTCGAAGCCAGGACAGGACCTGGGGAAATTATATG
TAAGTTTTTACTAAGATTTCTTTTCTTTAACCCAACAAGGCTCAGTGTGAGGCAGTCATGCCAATAATCCAAGCACACAAAAGTTAACTTTCTT
TTAAGAAAGTGTCATGAGAAATGTAAATTAAGCAACAAACAAGGGCTGGAATCCCACTGTTAGTACTTCCTTACCTCAGTGGTTCTTATTGTCA
GCTTGCATCAGAGTCACACTGGCTGTTTTGAGCCAAAAGATCTGGTGTCAGCCAGCAATCTGCTTTACTAACATTTTCCAAGTGATGCTGATGT
CAGTCCCCCAAGGCCCTCACTTTGAGAATCACCACTTTAACATAACTGTGTTAGAGGAGTGAATGGCAAAGGCGGCAATCTATATGCTTTAATA
TTGAATACATTAAGAGGTAGAACCTGTACTTACCTCATTGACACATACCTCAGAAAACAGCTCTAGGGTCTGCACGCCCGCTTCAGCTCACAAG
TCCAGTGGTTACCACGCGCCAGCATTCCTCCTACACTGCTGGCCACTCTTTGCCTTGGACTTTAGGAGGTCAGAGTAACGGTGCAGCTGTTAGC
TTGAGGGAGTGGCCACAGTCCCTTGACAAGTGTGCACACCATGTTTGATGTCAAAAGGAGAATCCGTGGCTCCTTTGCAAAGTGAATCTTCATA
GTACCATCCCTCCCTGGTACACTGAAATGTAATTTCATTTACAAAAGACACATCAGGGGAGGTACACCTCCAGTAAACAGTCGCTGAGCAAGGC
TCGGTTTTTATGGATTTCCAGAGCCTCAGTCCTGCCTTACTTTGAGGTCAGCTCATTACCATGTTAGCTTCCAAAAGTCTAACTACTTAACCTA
AGACAGTTTACCCTGCACTGATTCAAACCCATCAACAGTTTGGCTAAGTAAATCCTCACTTGGATAAGGTCACCGCACATACAGATGTACATAC
ATGCTTGTGAGCATGCTCTGTTTCCAAGCCAATGTAGACATTTAAAGTATTTTTCTTTCATTTCATTAAATAAATGCGAATATAAAATAGTAAT
GCTGATACTGTCCTGTCTAGGAAACATCCAAGAAATTGCTGGGCATTTTAAATTTGTGGTAAGGCACTACAGTTTATAACGTTTTTCTTGGATG
TGGTGAATGTTGTCATGCAAAGTAGCACTTGGCATTTGCAATTCTTTTATTATCATCATCGTCGTTATTATTTTAATTGAGAATGGCCGCTGTC
GATACAGTATTTGCCACCCAGCTATGTTATCCTAGATGAAATAGGCCAGACATACTTAGTGCTGTTGCTCATAGGAGGTACCTGGGGTTGTCAG
ACTCACACAGTCCATAGCGACAATAGTGGCTGCCAGCAGGAGGGCAGAGGAGTCGGGTGTATTTTTCAATGGGTACAGTCTCTGAGCTGGAAAG
GTTATATTTTGGTAGAAGGTGGTGATGTTGGTTACACAGCAATGTATATGTTCTAAAGTCTCTTATGTTACAGTCTAACATAATTTTTTTAAAG
CATAATAGCAGAACTCAGTCTTCCTTTCAAGAATGCTGGTCCTTGGCAGCAAAGTCACACTTTGAGAGGCTGCGGTGCCATCGTACACCGCACT
CTCATTGTGCTACTTTTTGTTTTCAATATTTTTTTTTACTTATTTTACATCCTGATCACACCCCCCCCTTCTCTTCTCCCAGTCCCACCCATAC
AAATCCGTCCCCCCATTTCCCCCTTCCCCTTATCCTCAGAGAAGCAGAGCTCCCTTGGGTGCCCTATCCTCGGACATCTATTCCCAGCAGCACA
AGGCACATCCCCTCCTACCGAGGCCCAACCAGCCAGTTCAAGTAGAGGGAATGTCCCATCGCAGGGGACACAGACCCAGACAGCCCCCTCTCCA
CTTGTTAGAGGACCCATATGAAGATCAACTAGGTCCAGTCCCTGATGTTTGGCTATGGGTCTCTGCCTCTGCTTCTATCCGCTGCTGGGTAAAG
CCTCTCAGGAGACAGTCATGCTAGGTGGCTCTCTGTAAGCCTAGCAGAGGATCATACTCTCAGGGGCTGGCATGCCCACATGAGATGGCTCTCA
GTCATTGGTTGGCTGTTCCCTCAGTCTCTGCCCCATCTTTATCCCTGCACATCTTATAGGCAAGACAGATTTTGAGTTGAAGGCTTTGTGGGTG
ACTTGATGTCCCCCTCCCACCTCTGGAAGTCCTGCCTGGCTATAGGAGGTGACCAGTTTAGTCTCTACATCCCCTGCTAGTAGGAATCTCAGCT
AGGGTGAACCCATACACTCCCCATATCCTTTCCCACTCCAAGCTTCCAAACAGTCCCAGAGATGCCCTCCCCCATGATTTCCATTCTCACTCCC
ACCCCTCTACTGTCCTCCTCCCTTCCCACACCTCATCTCCATGCCATCTCCATTCTCTCTCTCACCCCATCTCCCACCCAGTTCCCTCTCTCCA
TCCACATTTCATAACTATTTAGTTTCCCCTTCTGAGTGAGATTGGTGCATCCTCCCTTGGGACCTCCCTATTACTCTGAGTTGTAGCATAGTTA
TCCTGTACTTTATAGCTAATATCCACTTATACGTGAGTGTATGCCGTAAGTGTTTTGTCATAAGTGGGTCAGGGCTACCTTACTCAAGATGATA
TTCTCAAGTTTCTTCCATTTGCCTGCACAACTCATGATGTCTTTGCTCTTAATAGCAGAATAGTATTCCATTGTGTGATGTGTAAGTTGTTTCC
AGCTTCTGACTACTATGAATAAAGCTGCTAAGGACGTACTTGAACAAGTGTCTTTGTGGAATGGTGGAGCCCCTTTTGGGTATATACGTAGGAG
TGGTATACCTAGATCTTATATAGAGCTATTCCCACTTTTCTGACAAACTGCCAAATTGATTTCCAAAGCGCTTGTAAAAGTTTGCACTCCCACC
AGCAATGGAGGTGTGTTCTCCTTGTTCCACATCCTCACCAGCACATGCTGTGGCTTGGGGTTTTGATCTTGGCCATTCTGACGGGTGTAAGATG
GAATCTCAGGGTCATTTCGATTTGCGTTTTCCTAATGACTAAGGAAGCTGAGCATTTCTTTAGGTGCTTCTCAGACATTAAAGATTCCTCTGTT
GAAAATTCTCTGTCTAGCTCTGTACCCCATTTTTAATTGGGTTATTTGCTTTCTTCGTTTCTAATTTCTTGAGTTCTTTATGTACTTTGGATAT
TAACCCTCTGTCAGAATTAGACTTCGTAAAGATTCTTTflTAATCTGGTGACTGCCGCTTTGTCCTATTGGTGGTCTTTGTAGCCTTACAGAAG
CTTTTCAGTCTCATGAGGTCTCAGCAATTGTTGACTTAGTGCCTGAGCCATTGCTGCTCTGTTCAGGAAGTGTACTCCTCTACTAATATGTTCA
AGGCTATTCCCACTTTCTGCATTAGATTCAGCATATCCAGTTTTATATTGAGGTCTTTCATCCACTTGCACTTGAGTTTTGTGCAGGGTAAATA
TGGGTCTATTTGCAGACAGCCTTCCTCTACATGCAGACAGCCAGCTAGACCAGCACCATTGTTGAAGATACTTTCTTTTTCCCAATGTATGGTT
TTGCCTTCTTTGTCAAAAATCAAGTGTCCATAAGTGTGTGCGTTTATTTCTGAGTCTTCAATTCTATTCCACTGATCACCCTGTCTATTTCTAT
ACCAATACCATGCAGTGTTTATTATTATCCCTTTGAAATACAGCTCGAGATCAGGGATGGTGGCATGTGTGTTTCAGGAATAATCTGTAAGCTG
TGATATAACTGCTCCCAGGAGTGATGTCTAAACTAACTAACCCTGTGGGCCTGTAGATTTAGCCTCTGCGGTCATTGGAGACCATCACACATTT
TGGAGTCACTTCTGTTGATAGTTTTTAGAAAAATGTTTGATTTGATCATTTATAATAGACTCAGATCCCAATTATATTCTATTCATGGAACCAT
CAAATTAATTTTTTGTATACATGATAGTAGTTCAGGGGATGTGATGTTGGCAGCACGGGAATATACGTTGATATATTAATCACAACCTCCAGAG
GTTTTCCCCCAATCTCCAAATAAGCTGAGCTGGAGCGCACCGTAAAGTTCCTACCAAGTAATAACCTTAGCCGCCTAAATGCGTTACCAGTTTA
CTCTGACTCTCCAAGCGGCTGAGTGCTAGAGAAGTGGCTTTGTGGCTGCACCTGTCACTAGTGAAATGACACTTCTCACTATTACACCGCAATT
CAGGGTTCCCTTTTGTGGGTTCTTGGTCTCATThATAAAAAGATGCTAAAATGCACTCGGCGCCAGCAAGGTGGCTCAGCAGTTAAAGTAACCT
GACAACTTCAATTTGGTCCCTAGAACCTCCATGGTGGAAGCGAAGAACTGAGTCCTGAAAGTTGCCCTCTGACCTCCACACCTGTACCATGGCA
CGCGTTCATGAGCATGTGTATACACACACACACACACACACACACACACACACACACACACACACACTTGCACGCACACGGATTTTTTTTAAAG
AACAGATTCAGAAGGAAACTCAATGATCATTTTATTAGAGTTTCAGAGAAAAACAACTGGGTAGGCAAGCTATAGATCTCAGAAGCCGCTGGCA
CGACGACATCGAGAACAAGAGAGAAACCCAGGCCATTTTAAGATGCATAAAAGCCTCCATCTTGACCATTGCCATTGCGTGGCACCAGGGTGTA
GGGAATAGCATAGATAGTGAAACTGTCTGGAAGGTCAGTGAGCATTCTCCACACAGTGAGTGAACTGGGAGGAAATGCCCCAAGGTGTTGCACT
GCAGGCACACAGCCCTTTAGAGGCCATTTCACAGCACACACCTCCCTTGTGTCTACTTTACACACTTTTTTTTATTTTTAATTTTTTTTTTTGC
ACATGTTCTCTTTGTTAGATGAAACCCACGTCGAGAAAGGCTTCCAATCTTTCTGTGCCAACTGGCTTCATTCATATCTTAATCTTGGTAAAGC
AATTTTAGCATATATTCGCTTACCAGATTGCCTCACCCTCACATTAATGGTGAAGTGGCAACTTGCCCTTCTAGGAACCCCCATTCATCACAAG
AGCGCCTGCTATCTCCCACACTGGTGGCATACACTGCAGAGAATGGCTGTGTTGAGATGTGTCACTGCTCTTCTCCCCCAGGACAGCATCTGTG
ACGCTCTACAAACCAGTGGGTTTATCGAAGCCCCTGTGCGTTGTAGATTAGAAACTGTTGTGTCTAGATTTTTCCCGTATTCTACCCTTAGTTG
ACACCAAATCAGATAATTACATGAGTCTTATGTATTAACTGTTGTAATTTCTAGAAAAAAGTCAGAATCCAAAAATAATTGTCTCCTTGTGGCA
AAGCACAGCTCACTTCGGCGTATACCTTCTTGAAAAAGTAAACTCATGCATGGTTCACTTAACAACAGCTTTGCTCTGAGACCGCCAGTTTATC
CTGTGGTCCTCAGTGTGCACTCACACAGCCCACGCACACTGAATAATGTCATCTTCTGGAACTGCTCTCAATTCTCATTAGATCCGTGTTATCC
TACATAATCGTCAAAGCCACATAAAAGAATAAACGCAAATGTTCCCTCTAATCTAGCCACAAAGATACAACTCACCTACATCAGGTGCTTTTGT
GTGTCCTTCCAAACTGTTTTTATCCAAGAATTCTAACTTCATAAGCCATCCTCTCCACTGATTTGTACTGAAGATCTTGGGGGCACTTGGAAAT
AGCAGCACTAACCACACATCATCTTACAGGACGAAGAACTCCTCTTTCACGTCTTCCCTCCAGGTTCCATTACTCAGCTTTTACACAAGACACA
TGTGGACCTCCCAGTATCCTTATCTAGAGGATGCTTCTGATAGCCCTCATTCTCAACCCTACATTCGATTTAAAAACCATATTGCACGGACTTG
CAAAGCCTTTGCAGCTCCTGGGATCATAAAAGAAAGATTGGCTCTGTACAGATGATTTTAGTGTTAGCTCCACTCGCTAGGTCTGGTTACTTTG
AAAGTCATACTTAATGCACAATAAGCCAAATTGATGGGTATGGGGAAGGCTGACTTAGCTCAGTATTATCCACAATCTAGACAACACACCCTCC
AGTAAAACTCTAATTCCATCCAAGTGCCTGCCTTCTCTTGTTCTGATCCAAAGCTTTCCCTCAGCTTTTCCTTCTAGTTCCCCTCCCAACCTCA
AAAGCAGTGTCTGTTCAGAGCGCTGGGCTCTGGTGTGCGGCTCTCTTACTGCCCTGTGAAGTGGTTTCCACTTAAGGGTTGGGGTGACGCCTCA
CACTTAGACAGGGTTAAGCAGGTTCTAAAACACGCAAAGAAAGACCAGGTGTGACTTTAGGGGTTGCTTAAGCCTCCAGACTAGTACCATCCAC
AGTCCTTACCTTAGAATAGGTATTTAGTAAGCAGCTGCTGCATGCAGTTGGCGTTACAAAATGGGTCTTTGGAAATTTTTACCTTTCCGTGTAT
AAGACAATAACTTCCTTGAAAGCGAAGACTTATTTGCATCCCTTCAAATCCTGTGTCCTTCGTGGAACATAAACTCTATATAGCTTCATGTGAA
TATTTCTGTGGCAATCAGGTGGTCACGGAACCAATGCTTACATTCCACTGGGTCTTTTTTTACTATCTGCACCCAGCAATTGATGGCACTTAAG
TAAATATCCTCAATTGTGTTTGACATCCGATTGGGTACAGGTGTAGGGACAGGGCAGACAGTACCTCGATCCTCCCTGGTGAAGGAGTTGCTAA
TACTGACGAGACTTGCCTCCTTCGTTTTCGATTTGGTTGCTTCACTTCACTGAGTCACAGCACCTGCCACTTAGGATGGCCACACGGCTAAGCC
AGAAACCTTCCTTCAGATTGCTGGTGACATTATATATGAATCTCTGGTGACATTCTCGGCTGACATTTGTCGCCTGATCTCAAGTGCTTGCTAA
AAGTGAGTTAGCTTTGTCATTGACACGGAGACTAACTTTCTCTAACCCAGACTAGTGAAAACTGAATTGTGCCTCACCACACGAAGGTTTTGCT
TTCACTGTGCCCAGACCCTCCCATCTCTCACAGCACAGTCATTTTGTGTCTGATTTGGGGGCGGTGTGGTTGTTTCTCAGTGGTGTGTGACCCA
CTTTTTCATAATACTTCATTCCATGTGTTTTTACAACACAAGTCAGAAGCTGACGAGAGTGGACTGAAGCCCCAGGAGCATCTTCTGAGGCTTT
CTCGCAAGCTCTAGAACCACATTCTAAGAAGAAGGTCTTAGCCCTGCAGGGTGTGCGTCCCCTACCAGAGAGTGATGGGCCAGTTCCTCTAGTT
TCTGGATAATGAGCCAGTGCACAGGTAACGATGCCTTTCTCACGGTTGATTTCTTTTGTGTCCCAATTATTTTACCAAATTAGACCTTTAAAAA
TACTAGCCACTTACCTGCATAGCTTCTCTGTTGGGATGGGGAGGGGCTCAGGAGAGTACACATTTTCATTGATACTTTGAGACCTTAGTAACTT
TTCCTTAAGAATCTGAAGGAATCAAAAACTTATCTCTGTTTTCTGTGTGCCCCTAGCAGACGTCCTCTCTGATCGTTGGTTGTTTCGGTTTGGT
TCTACTGCTTGTTGTTGTACTTGTCTGCCAGCGTCAGATACCTCACTCCGCTGTGACATCTGGGTGATAGTTAAACCTTGATTAGCTTCTGCCT
GGGGTATGGGCAACCTGTGGCGAGAGCAGACAGAAAGCTTCAAAGAAGAGAACGGCTAATGAGGGTCCGATGTGTCTTGCTAGACACTGGCTAC
TGCAGGCTGGAAGCAGACAGAAGCCAAGTGCTATTGGCTTATGTTTGGCAAAAACCATTCCTCAACACCTGTTTATAACTTTATGTTTCTGACA
TCACAGCTGTTAACCAGACGATTCTTTTAAATATCGTCGTTTCAAAACAAAATGTTGAAGGTGTCACCTCTCCTAATGTCACTTATCCTGATTC
ACAGGACAGTTTTTGGTGAACATTTATTTACCAGGGTCTGTCTGGCTTGTGGCAGTATCATCTGCACACTGGACTGCATACCACCTTAAGATGT
GGAGCATGCCCAGAGATAAGTCAACCTTGTTTTCCATCAAAGTCCTGAGACACAAAAACAATGAAAGTACAAACAGTGACTGGAAAAAGTGTCA
CACACTTGAGCCACTTTGCATCCCTTGTTAGGACAAACCCTGGGCTGGGTTTTTACATTCGTGAAGCTGTGCCGTAGGTGCATCAGTGTGGGAG
GTTCAGAAAGAGTAGCGGTTAGAATAAGATTTTCCATTTATTGCCCTTTCTCCATGGCTAAAAATAAGATTCATACAAATCAGAATGTGTAGTG
CAAGCAAAATCATTCTGTTAATTCTGATGACTGGCCTGCCCACATCTTTGCATCTGTCTGTTAAGTGGTCCATTACGAAATAAATGTCTCTGTT
TAAATAGCAAAGGCAACCTAGGTTTTTTTTGTTGTTGTTTTAATTCTATTTCTCTACACATGATAGTCTGTCATTTTTCCTCTTCTAAACGAAG
AAAGTCATAATTCACACATACACGAATTGTGTTGGTGACTGAAGTGGTGAGATCCTTGGCTGTGTCTAAAAAGTACTCCCCTCCTTTGAAAGGT
CAATTTACGGTGTCCATCTTCAAGGCTTATTGCTAGAAAGTTATCTCTCATGAGCCCATACAGTGGTGAGGAGAGAGGGTCACTTAGCTGTATT
GGAAACTCCATGAGAACATCATCCAGTGCACACCCGTCCTCCACCGAGCTCCATCAGCACAGATGTTGGGAGGAGCAGTTGACCAAGTGAAAAC
TTCCCTTCATCTGGCTTCAGAGTCTCCTGCAACCACAGTAAGTGATAATGTCTCCATTTGTGGCTAGACGAAACACCAAAGAGAAGTGTCGGCA
GTCTTCACAGGGAAATTTGTACCTCAGTGATCCCAGCATTGATGCTGTCCCATCGCACCGACTGAAGAGCGGTGGCATTTCCACAGCTGTGTCT
GCAAGCGCACCGAGCCTTTCAGTTTGATGCGGTCCATGAGGTAGATGAGTCAGGATGCCCTCTTCACGATGCATCCTTCAAAGGCAGTCATTCA
GAAGCCTTATCAATGGGAGCGGCAAATCGCACTCCTCTGACTGTGACGGTCCCCGTGTGCCGCAGCTGAAGCAGGAGCGGTGGGAGACGACAGA
CCCCTGCACAAAGCTTAGCCGGGAGACCCACGCACCTGGGCCCATCCACTTCTCCTCCTAGACCTCAGCATCCTTATCTGGAAATGAGAAAAGG
TGCAGTACCTGCCCCGACGGGTGTCTGTGGGATTTCATCAGCTAATGATGTAAGATACAGAGCCAAAGCTGTGCGCAGTGCGCTGTCCGCGTGC
GGCTGCTCATTCATGTTCCCTCCTGCATCTCGAAGGTGTGCTGGAGCACGGCAGAGCATCCGAGGTTCTCCCCTATGTGTACAGTAGAAATTTT
AGGATTTAATACACAAAGGCTCAGCCTCTGGGTCTTCATTCCTCACTTAGAAGGTGAGAGCATTTATAGAGCACTTTCTTTTTCTTTTCTTTTT
CTTTTCTTTTTTTCATAGCCTGCTAATGCTGCCTGTCAGGTGATTTTACTTTCTAATGACATCGACATGGTGTGCAGCCCTTCCTTTGTAGATT
ACCACCGAGCATATTAGGCTATTTTTATCCGCACACTATGCTTTTCATTTAAACCCTAGTATTTCAGTTCTAACATCTGTGCGTGTTAGCTGTA
GTACCTCCTCGTGTACATTAGGCGAGCGGCTGCGCTAATGCTGTAATCAAGCCAGCTTTTAAATATTTCAGGTAGGAACTAGCAAAATCTCAGG
AGCAATTAGCTTCCATTGTAGTTTCCCTCCCTAGCGAAAACATTGCCTACGCAGTGCTGATCCATCATCAGGGTGACCAACGCTAACAGGCAGA
AGACAGTCAGCACTGCGTTCATTAGTTTTTGTTAATTATTTTCTGTTTCATGTATATGTGCCCTTCTCTGTCTTTTTCCTCCCAACAAGGTTTT
CTATTACAAGTTAAATATTATTTTATAATCTAAGGGCATAGCTATAAGCAGCACATAGCTCGTGGTCACCCTTTCGAAGCCTTGGGGTTCGACA
GTGAGAGTTCAGACTAGACGTTGCTTCAGCCTCCTCTGTGACCTTCTGGGATGCGGTTCTCATTCATCAAATTAAGAGAGAAATTGTGACACTA
CCTTGACTTTGCCTCACCTCGCGCGCGATGAAAGTGATGAATGGGACCAATAAACACACAAGTTACAGCACAGCCCAGAAGCAGCCAGGGTTCA
CTGTGAGCAGGGAACGGATTTTAACTGCAGACTCCCTTTTCATGTTCATTCTGCCATCCTACCTACCAAGGCTGATTGTAGCATACATATTAAG
GTCAGCAGATTTTCTGGGTTTCAGTGTTAGGACCTGGCAGTCTGTAGTGAAGACAGACATTCCTCAGGTTAAGGACCAAGATGGTTTGCCTTTT
CTTACAGCCATATCTCATCCCCCACATTGTATCCAGGCTGTGCTTTATATGCGTTCTCTCTCTCTCTCTCTCTCTCTCTCTCTCTCTCAAGAAA
ATCCTTTTCTTAAGAGATTCTTCTTAAGAAGAATCCTTTTCCTCAGAGAAAATCCTTCCAGGTGACTTTCAGAGTTCCTGGCATTACAGTGCAT
GCATGCACAGGCACACACGCACAGGTACACATGCGCACACTGGCACACAGTGGCACACACATGCACACACTGGCACGCACACCATTACCTTTGC
CAGCCTCACTGCACTTTGCCACTGAAGCGTCTATGAGAATTATAGACTGGAGAAGGTGATAAATTAAACTCTGGCTGTGATTTCCTACCTCACC
TTTGGGGGGGATGACAAAGGATCTGAAATTCATCCGGTTGATACACTCTACTTCTTCGTATGTAACAGTGGTTTTAACCTGTCCTTCATGTGCA
GTTTGAGGGTAGTGGCCAGGCTGGGCCCATTATTCCTTTCCTTCAATAATATAAAGAATTTGCAATCGAGATTACACTAATCATGTATTTTAAG
TGCATGTCCGCATTGGTAGTTTAGTCCTCTCAGTCATGAAATGCTTTGGAGTTTATTTCTAATGGGTAAATGCTCAGAAAAACTGAAATTTTCA
ATAAGATTGACATTTGGAGTAAGAAATATAAATCTAATCAGAGCATTTCAACCAAGGCTTGTAATTAGGGCTCAATACAGTAAATTTATACATG
CTCGCTGCAGAAGGGCACGGCGGAAGGTGAAATTGCATGCTCTTCTGTGAAGGAGCGACGAAGGCCAATGTATCAGTGTCAGGCCGTGACACCA
GAGTATAAATGACACTGTCACCAAGTGCTGCACTGCGTACCAAGGGAATTCCACTCGAGTAAATTACATTACTTAGACAGACCTGAAGGCACCG
TGCTGGAATGAGTGCAGAAAAGGAGAAGTCTGTCACCACAAAGCTGAGAACGTTGCCATATAGCCAACTGCAGGATGCACAGCGCTGGCTGTGT
GGGACCTGGAAGGGACAATCTTCCGCTCTGTTGTTAGCTTATAGACGTGACACATATGTACAGTTGACCTAGAGAATGGGTATAAGCTGATTTC
TGTGATCTGGTCAGCATTGGGTTTCCCTTATTATTTACAGTTGGGTGGCAATAGGGGGTCAGTGTACATTTGCCATTCTTTCTTTTTCTCTGTT
GTGAGAATCTAACCCCTGCCCCCTCGGTAGCACTGTTCACCTCATTTCCAGCCCCACTGTCATAAAACACATGGTCGCCATGGTCAGGTGGAAT
GTGTCTGTAATCCCACCTACTGGAGAGACAGAGGCAGGAGGATTGCATAAGAACTCTTTTTCTCACACTGTGTCTTTCTGAGAGTTGATCTGTC
TTAGCGTTTCTGGGCAGGAACTGGGAGAAGACATGAAACATCAGTGATGACGCCATCTCCTCTCCTCACTTACTGAAACATGCGCTGGTCTCCT
TTTGTCTGGCTGCCATGCCAGTCAGTCCCTGAAGAAGCCAATCACACGGACTCTAGAATCAGTCACTCACTCCTTGGCTAGGAAGTAAATTCTC
CCAGAGAGAGTAGTCTGAAACATCAGGGTGGCTTTCTCAGCCATGTGTCACACCTTATCCTTCTTGAGACACCCCTAAACAGCCTCTTGGGGGA
CACGCTGCATGGAGAACATGGACTCTGCAGGTTATCTCCACACGGGTTACATGGCACGTGTTCCGCCATCTTCCTAACACAGGGCACATTGGAC
ATCAGAGGTGGCACTCAGGCTTCAGTTTTAGTCTTTCCAAAATAATGCTCTGTCACAGCTGGAATTTCATCTGCGTTTTGTGCCTCTATAGACT
TTATTCATTTAATCATGGGATTTAGGAAAGCCACCTTACTTGGAAAACTAGGTTTGAGGGCAGGAGAAGAGAAAATGACATATTTTCTAATTTA
TTTAAGTATACCCACCTTTAAATTACTTCAAGACCCTCTGGTGCAGCTCAGAGGTAGAGTGCTTACGCAGGACATGCAAGGCCCTAGGTTCCAT
CCCCACTAATGCATAGGTATGGGTGTCAGTCTGGGATTCTGAACTGACATCTGCGTAAAAGCTTCACACGGTGTCCATCTGAAGCCTGAGCTCA
CTGAGCGCTTACTCATCAGCCGTGGCAAGTCAGCCTCGCCTGCACTGGCATGTCCCCGTTTCCATCGCATTTCACTTGTGTCCTCCACAACATC
AGTAGGGGAATTGTATCTGTGTCTTGGTGTTTACTACAAAGTTACTACGTGACGTTTTTCCCCTGGCTTCTCCATGACAGCCTGCTACCCGAAC
TACAGCTCTGAGCTCCTGTGAGCTGAACCCTGGTGAAAGAGCTTTGACAGCATCTAAAATGTTTGGAGCCTCCCTCCATTCTGAAGAGCCTCCT
TTGTTCCAACTGCCCTACCAGTGTTGTTGTTGTTGTTGTTCTTCTTCTTGTTCTTCTTCTTCTTCTTCTTCTTCTTCTTCCTCCTCCTCCTCCT
CCTCCTCCTCCTCTTCCTCCTCCTCCTTCTTCTTCTTGCGTCCAAATTGGAAAGGGAGCAGCGGGCTCTCCTAAGGAGGAACGGGTCTGTGGAG
GATTGCGAGCACTCATTCAGAAGCTATTCTTAGTTCCCAGAACTATAACGAGGCTGGGCTTTTTGTCCTGCTCAACCTCCATTAAGGAACGATC
CCTCTACCTGCCACCTGATTCTGTTTTCCCCAAGGACGCCTGGTGTTTTCTTCCCTCATATTCTGAATCGACTGGCTTCTGAGTCAGGCGCATC
TTGCAAGGCAGTGAGTCCTGTTGCCGTGATGGCTCACCTACTCCTCAGCTGTCCTCTGAAGTCTGCATTAGCAGCGCTTGCTGCAGACCTGGAT
TCATCGTCAGAAGTCAGCCTGTCCACGGCAGCAACTTACCCACATTGTCACGCCAGTGTTGGCAGCTGACCACTGCTGCCTTGCACTGCTGTGT
TCCTGGGCTCCCACTCCTGCAGATGAGAAAAAGAATGCAAACAACACAGTCCCCCACCAGAGCCCATAGCCATGTTCCAGAGGACAGTGAAGTC
ATTGTTCAGAGGTGACCTAAGGACGAAAGCATTTAAACAATAAGCCCTCTTGTGAGATGTGGTGAACTATGCACTGCAGACATTGTGAAGTCCT
CTGTCTCGGCGAAGGCAGTTTCTGCACGGGTTTCAATACCTTCTTGACACCCATTTAGCAGACGAAAGTCACTTGTCTCACAATAGCATCCAAG
CAGTTTCTGTCCTAAGGAACGCTAAACTGTGTTCCTTTATATAAAATACACACTGTAATTTATAGAAATCACAGTCCTCCATTGAATTGCAGTC
CCCTCCCCCTCCCCTCCTTACCTGAAGTAGAAACTCCATCTGTTTGGTAAAAGCCTTCTTTCTTTGAATTTTCTTTTTATATTTTGTGTGTGTG
TGTCTGTGTCTGTCTGTTGTCCACACACATGCACATGTCACTGAACACGTATACCGAGGTCACAACACAACTTGCAGGAGTCAGTTGTGTCCTT
CTCCTGTATGTCATGGGGATTGAACTCACATTGTTAGGCCTCCTACCAGGCCCCATATTGAGCAGCTCGCCCACCCCTCTTCATTCTTTGAATA
ATATTTATTTCTTCCAAATGAAAAAAGGGATCCCTTCGGTTGCTTAGATTCCTAATGAGGCTCTGAGGCTTGATAATAATCCTCGGAACACCTT
GTCACTGTTGAAGCATGGGAAAAGAGGAGCCCTGCTTTGAATTTAGTTTCGTTTGCATTCATTTAATTTAATATTTTTTGGTTTTGTTTTTGCA
TTGAGCCTTTAAGGGAGGCTTGGTATATAGACATATACCCTTGTTAGCTCACACCAGTATCTCTGGCAGAACCCTGGAATTGTTTCCGTTTTGA
TGTTGAATATATGACTTTCCGTGATGGTGCAGGGCGCGGTGCATATGTTGACTGGCCAGAGGTGTTCTGTCACGATATAAATGTAGGATTAAAC
TATTAAAATTGCCAATTTGATGTGTTTGCAGATCACTGTATACAGCACCATTGCAGAATCCTCCTAATTGAAGAAATGTTTCATCTCTATCTCT
GTTGACAGGAAGGATCGCCTCCTGATAATTGCTTTGCTTAAGTGTCTGATAATATGCCAGGAGTGAGCGCATTCTGTGTTAGGTCATCTTTAAT
TATGCATCTACCACCTGTCAGCTCTAAATCAGGATCAGATCAGATCCTTGCAGAGTCACTTTGGGGAGGGTCGGAGAGAAGCCAAGGTGATCTC
TTTGAGAAACAACAGAAATAGCAGTAAACATTTCTCATCACCTGAAAGCGCCTCGGGGGCATGGCTCATGAGGCACCAGCCACTGGCACATCTG
AGAAAATCCGAAATCTCACCCAAGCAAGATAAATTACCTCGCAGACAAATTACGGAGAGCTTGGAAGTGCCCCCTCCCTCCCTCCCTCCCTCCC
GCCCGCCATCTGTCGCACTTCCTTTTTATTCTTTCTCATCCTTGTAAAGGAGCCAGGGAGAACCTCAGACACATTTTTATAAGCTACGTGAGCA
CAGGAAATATGATAATGATGTTAGATTTCATACATTTGGGGGATTTTTTTTTCAGTAGTTAGTTGGCTTTTTTTAGAAGGACAAATGTGGGTTG
GAAGAAATGCTTGCTACCCTCTGTCACCTTTTAGTAGTTTTAGTCTCACTTTTCAGGGCTGTTTGGGACTTTCTGGAGGGATGTTTACCGTTCT
TTACCGAAAGTTCTGTTCCCTCCACTAAGAGGTGTTTTTCTCTGTTCACCTGTCAGAGGCTTAAAGCCAGAGTGAACACACGTTCACTCTTTCA
TATAACACGTGGGCTTCACTGACTGCACACGGGCAGGCAGAGAAGGGGCAGGCGGAGAAGGGGCAGGCGCAGGGAAATGACTGAACATGCTTTA
TCCCATTGTGCAGCTCTCAGAACACAAAGCGGACGCTCCAAGCCTGACGTTCTCCAAGCTGGGGGGGTGGGGGCAACGCTCCTGACATGGCTTT
GCCTTTGACCCACTTTTGGAAAATCCTGAATTTCTCTATGTCAGATCCAGCTGAATAGAGGGACAGGACCGCCATAAAACTGGACCTCCAGCCC
CACAGAGCATTGGGTTATGACTCTGAATCCCTCTTCACCACCATCCAGTGCCTAAGGTGACGTTTACTGCAGTCGGCATGCATCCAGGTAGCAC
AGAGTAGGTCTGACACTCACTCTCCGCTCCAGCGTTTGTCACTTAGTGTGCAATTGCACAAGTAAAAGTTACACAGGACTGTCAGCTCCACACA
GCGCAGACTTCTCTCAGAACTGACTGCAACGTGCCCATGCCCGACAGAAGAAGATCCCAAAGTTAAGTCCAGGAGAACCGAGAGAACGAACCAA
CAAGTAAAAGCAGTGCCACCATATTTTCTCATCCCCGCTCGTCTTTGCCAAACCCTTTCCATCTGCCTGGGAACATTGTCTGCTCTCTCCACCT
GCCTGACTCCTATTTTGAATGTTATCTCCTCTAAGAAGCATCTTTAAATTTATTCTTTTATAATTTTATACATGTATATAGTGTTCTTTGCTCA
ACTGCCCTCCCTCCCCAGTTAACCCTCTCTTAATGCCTTCTCCCATCACCAAATCCCTTCTTCTCCTCAGGCCCCCCTCCTGCTTTCATGCTTT
TCTGTGCGTGTAAGACGCACTGCATTTAATTAGAGGTGAACATGAGTCAGGTCATTTGCTTGCACAAGAGCAGTGTGCCAGGCTCATTCACAGA
GGAATATGATTGCCATCTCTCTGTATCCTTTAGCTTATTACATCTCCTTTGAGAGTGGCGAGGCCTTATGAGCCTCTCTCGGATCCATGATGGA
GAACTAACTGGTGGGCCCAGCTCTACGAGGGCAGCCACTGCTGCTTTGAGTCCATAGGTGCGATGGCCATGTCCTGTCCCCTAGACAACACCTA
TAGTACTCTTGGCATCTTCCAACTCTTACTTTCTTTCTGCTTCCTCTTTCTTGGCATTCTCTGGGCTTCAGAGGCATTGATACACATCTCCCAC
TTAGCTGAACATTCAACAATCACTCGTTCTCAGCACATTGACCAGTGATGCGTCCCTGCGTTAACTGTCACCCATGTGAATAGAAGCTTCTCTG
AACAAGGCTTAGAGCAACGCTAATCTGTGGATGTAAACATAAATCTTCCGAGTTCAGTTTGAACAGCCAGGTGTGGTGACACACGCCTTTAATC
CCAGCACTCGGGAGGCAGAGGCAGGCATATTTCTGAGTTCGAGGCTAGCCTGGTCTACAAAGTGAGTTCCAGGACTGCCAGGGCTATACAGAGA
AATCCTGTCTCGAAAAACAAAAACAAAAACAAACAAACAAACAAACAAAACCATGCAGTTTGACACTGTGCTCAGTTAGCAAAGAAAGTTCCCC
TACTCGGTCTATGACTGCCATAATCATGGAGTTTGATGAGGTTTGTAGAACTAAGTATGAATTCCTCCTACAGGGAGGCACTCAAACCCAATCA
GAAAGTACCTCGCTATCCCTAAAACAATTACACCACTGTTGCAATGGTGGGCATATCTTGCCTAGCATGTTGGTAATATAGTTCAGAGGTTCAC
AGTTAGGTAAGACCAGTGGTCACTTTCTTCCCCTGGCAGCACTCTACTAGCCAGCACGAAGGAAGCTTCCACTTCAGTTCCAGCTGGATTTCTC
TCTGTTTTTGCAATCAAAATGTGTCATGTCCTCAGCAATAGGGTCGAACCATCTGCTTCTGGTGGGCACCCAACAGGAATGACAAGGGCCCCAG
GGATCCCTCTGATCAACAACTAAGGAAGTACCCTATACCTCCCACTGGGGTTTTGTCTAATAACCAATGTCTTAATCAACATATTGTTTTCTTC
CTCCTGCCCATCACCAACTCCCTAGCAGACTGCTTGTCTGGCTTTATCAGAGTTGGTCATAGTATTCTACAAAGTATCCTTTATCGAGTTTTCT
CTGGCGAAAAGAAGATACCATCTTAACTGTGACCAGGAAGCTCCAGAGACCAAGCAGTAATATCTGGTCCACAGTCCTCAGGTTACACTAAGGA
TATTATGACCATACATTAGAGAGGGACATACACACATGTGAGCGCTACATGCAGTGCACAGTGCAGGGATGAAGGACTGCGCCTGGGGAAGAGG
GAGAGGACGGAAGCCACAGTGGCTGTGTTTAGATAAATGACAGACTTGCTTAAGAGATGGGCATCATACAGAGGAGATTTTCGGTTAGAACATG
ATTGGTTTGCTTGTAAAGCTAGCCTACTCTGGAACCGATGACTTTCTTTGGTAACAGTGGCTCTCGTTTCTAGAAAGAGAGTTTATCTTTTAGG
GTTTCTATGGGGTTTGAGAATATAAATTTCTGGGCTCTGCGGGAGACACAAATCAGATAACCTAACACTGCTAAGATGGAGTGTCCCTGTTCCA
TGCAGCCTCCCAGCCGGGATGGAACACACTCCTGACTTCCATGTTGTATCACTTTGTCTAAAACATATCCATCCACAAGCTGCAGGCTAGAGGT
TGGGATGTGATGGAAGACTTCCTTTCCCGATGAGGGTACTGTGAGACAGATCAGCACTCAATCCAGCCACAGTGCGCATGCCTCCTGCCTCCTT
GCTCGATCGGACTGAGAGCAGCTCCTCAACACTTGAACCCTCCATTCTGTCCAGGAAAATACACACACTATTGTTCACCTCCTGGGAGTTGCCA
TGAGGGTTAATGAACTCCCTGAAGGGGCCACCTCAGAACCCATCTGTCACCAGTACAGTGGACACAAGAACTCCTGTCTGTAGGGATTGCCGTG
ATGAAACAGTGTCATGTAGAAAAGTCCCCAGCTTGGAGATGGATAAGGCCCTCCTGGTTCAGTCCTCAGCCCTACAGAGGAAGAACAAGAGACT
CCCAGCTGCATACCCGCTGCAGAGTAGGTTCCTGGGAGTGTTGGCTTCTGGGAACAGAGGTGTTCCCAGCATTCCCTGGGACCTGCCAGTGACC
TGCAGTGTGCGGCCTCTCTGCTGCTACCTGTTATCTCAGCCCAGCACTGGCCACATCTCTCCTCCTTCACTCCTTCCCTCTCTACTTCCATCTC
ATCCCAATCCTTCTTTCTTTATTTCTCTGACTTCTCTCTTGGCCGCGTCTTCTTTCCGTGTAAGATTGTTCTGATGACCCCAGCATGTCAAATG
ACTCTCGGTAACTCTTTTATTCTATTGTATTAATTTTTTAATTGAATGTATAATCAAACCTGGGTCCTCTAGAGGGGCAGCAAGTCCTCTTAAC
CACTGAGCCATGGCTCCACCCCCTACATATTTGTCTTATTTTGCATGGTTATGTGTGTGTGTGTCCTTTCTCTCTCTCTCTCTCTCTCTCTCTC
TCTCTCTCTCTGTGTGTGTGTCTGTCTCTGTCTCTCTCTCCCTCTCTCCCTCCCTCCCTCCCTCCCTCCCTCCCTCCTTCNNNNNNNNNNNNNN
NNNNNNCCTCCCTCCCTCCCTCCCTCCCTCCTTCCCTTCGTCCCTTCCTCCCTCCTTCTAACTCCCCTCACCCCTCTCTCCATGGGGGTGGGGT
GTTGTAGGTGTGCATGCGGAGGTCAGAGGAGAACTCAGCGGAACCTGGAGCTAGAACTAAGGTCGCCAGGCTCAGCAGCAGGCATCTTTGTACA
CTGAACCATCTTCAAGCCAACGTTTTTAGTCTAAGCAAGAGGTTTTTTGTAGCTTTGCAACGAGGGAAGAAGATGAATCTTTTCATGTTCCTCT
AGACTGCCTTCAGTTCTTTTTTCCCCCTGTGTTGACATTTTCCCCTTCTCTCATCCCCTCACCCTGTAGTCATTAACTTGCCAGACACTCAGCC
ATGGATTGTCTTTATGACGTTGCTCAACTTAGGAGCGTGTCATTCACAGAGAACCTAACCCATATATTAACTCAGGCACAGCAAATTGCTAAAT
ACATGCCTGTAAGTCAAGAGCTTCAGGATGACAATCCAACCACTCCTATTGGAAGGGGGTCACTGAAACAGACTGGCAGTGGGCAAGGGAGGGT
GAGTTTCTGCCACAGAACTGAGCTGGGGCAGAACACTGCCCTCTGGACACATACAGGCGCCCCAGCAGAGCATTTCCCAGGACGTGATGGCGAG
GCCCCAGTCTAACAGGAAGTCAGTGGGCTCCTGCCAGCCTCCACTTGTTCAGTCCTTCTCCACAACTTGAGCATCCACACCACGGCTCATTCCT
CCTCCTAGTCCCACTGGAATCCCTCTTACTCCTCCCACGGCCTAGTTCAGTAAATGTCAGAGTAGCTACAAAAGATGAGCGCTGCATTAGATAC
AGTGCCCAATGGATACGTGAGACCAGATCCACACATGCCCTACAAGTGTTGAAGAGCATCAGTACATGTGGAAATCGGAGCAGCATCTAGTGGG
AATCATGAAGTACCCTGATCCACGTGTCTGTGCATGCATCAGGAATGGTGCAAGAGAAGCCACACAGAGTCTCCCGACACAGAGTCTCGCGACA
TGCAGCTCTTCAAAGCTTTTGTTACTAGTCTATGCTACATTTTATTTAAGAATGTTACTTTAAATTTACAATCATTTTTAAACTCTGAATCAGA
AGGTGGTATTGGTTCTCAGAAAGGAAGACAGCGATCTATTTTCCCAACTCTTGGAACATGATGTTCCTTTGGGAGCCAGTGCCCTGATGAGAAG
CCCAGGGCTCTAGGTCCTGAGGTCATTACCCACACCTTATGACTCAGTGTCCCCCACACCCCAGTCTCAACACCTCTGTGAGTGGTGAAGGAAA
GAGTCCTGCCTTCTGTATAGCCCTGGGCTTTGCCAGCAACCTCGTGAGAAGAGATGTCTAGGAAAACACACAGAAAGCAGGGTTGCTGCATGTA
TGTAGAATCTCACTACAGAATCTAAAGCTTCGTGCTTCTCAGAACATTCACGTGTTTGTAAAAGCTTCATGCAACACTTGAGGTCAGTTGCTGT
GTGCAGTCTAGTCAGCCACCAAATCCATGTAGAAGAGGCTTTTGTTTGGCCTCTTTAAAAGAGAAGTAACTGCTGATTTTTCTCAAAATGTTCT
GACCTTTGTCTTGTTCACAGATATTCATTTTCTTGTTTGGTGTTTTTGTTTGTTTGTTCTTCCTAAGCTGGTGCAAAGGCACCCAAGCTTTCAG
TTGTCACTCTTCTTCCATTTGCTGCTCTCGCCTGAAAGAGTCACCACAATGCTTATAAAGGCCTTTCAGAGTTCAGTCCCCACCTTCAAGCAGA
GGACTCAGACAGATTCAGATAGCTGTGCCGTGCGTGCAGGTGGTAGGTTCCAATGTACTTGGGGCTATGGGCATACTTGGTAGCGATAACAACA
TTTTATAAAGAACAATAAGATGCCCCAGGTGTTGTCTTAGTGTGTATATCTTTGTATCTGCCTTGTCCTCCTATTCTATGCCAGGTCCCTTGAC
AGCAGGGGCTTCCTGGTTCCGCCCTGTGTTCTGTGGTGCCTTAGCAAATCGATGCTTAAACAAATATGTTCTGTACCAAGCTTAACTTTTATCT
TAAAAATAGCATAACAGTTTTTTAATGTATTTCATTTCATAGAAAAGCAGAGCCTGGATACTTTGCAGTATTTTTTTAAGTCTTGGCAGGGGAA
ATGCTAAGTCATGAAAAGTTCTCAATCATTGTAGAAAAAGTGATTTTGTTTATGCTTTGACACAACAGGAATGCAGAATTTTTCAAAGAACCTT
GGCGATCACTTGCTTAGATTCTCTCGTGTAGCCGTAGTACCGAGAACTATGCCTGACATTTAAAGGAGGCCTAATAGATATTTGTTGGGTGTTG
CAGAGATGGCTCAGAGGCCAAACCCATCTCTCACCAAGCCTCACAATCTGAGCTCAGCCCCAGTCCACAGCTACTAGAAGGAAAGAATCGACTC
CCTCAAGTTGCCCTCTGACCTCCAGACACTTACCAAGACACGCATGTATAAAATGTAATGATAATTTTTTTTTATAAAATACATTTGTTGGATC
ACTGAGTAAACAGCTAGTGAGCTGCTCCTGTGTGCTAAGTACCACATGTTAAATTCTGGAGACTGAAATAATAAATAATGGATTTGCATACATA
CCCCAGTACTTTCTCCCTACCCAAGAATCAATGTAGGAGACTAGTGACCCCTCTAGCCATCCTCATTCAGTGTGAGAAGGCAATCAAGATAATC
CAGAAATTTTATTTTTGTTGTGTGAGTGTTGTTAACCAGATATCATGGTTATCACTTCTAATCCCATAAGAAAGTAGAAGGCTGAGGTAGAAGG
GTTACACGTTCAAGACCAGCCTTGGCTACTTAGCAAGACTATGTTTTAAAGGTCCAGTGGCTGTGGACCTCAGTATAAGAGGACTTTTATCTCA
GTATAAGAGGACTTGCTTCCCGAATACAAAACCCTGAATTTGATCCTCACCACTGCCCAAAGAAAGCAGAAAATGTTATATCGTGTCCCTGAAA
CAAGGAGAAACAAACAGTCGGCTTATGTCACTCTCCTCTTACGCCTGTCTTACTAACCTTTGTCCCAAGTTGTTTCATTTTCTGGGCAAAACGC
GATCAGGATTCCCCCAAGATACAAATGGTGCTAAAGGTAGCATGCAAAAGCTACACCTCAGACTTCCAGGCCTAGGAACTCCCAGGTAAGTTAG
TCAGGCTCTCTAGATGAACAGAGTTAATAGATCTTACGGGGCATCTATCAGATCAGCTTCCACAATGTGGACTGAACGGTCCAAAGTTGACGCT
AAGCATTCTGGCAAGGCACTTCTACAGGAAACTAATGAGATGGGGCTGAGCTGCGACAAAGACGTGGTCAACAGCAGGGAACTACAGTCAGATA
GTTTAGCCTTGGGAGTAAACGTATAATTGACCCCTCCCTCTTTCTCTCTCACCCTTTCCCTTACTAATGTTTTGGGGTTTCTCTCTGCAGCCTG
TGGTTTGACAGCTTCGTTCCCTAGCTGTTCTCACACACTATGTCTTTACCCTTGCCGCTCAACCAACTGGCACCCTTGGCAGGCTGCCTCTCCC
TCCTGAAAGATCTTCCCTAACCTCCCCCCTTGCAGACAGACTTTCTAAAATGTGATCATTTAGCCCCTAACCCTGAGCTTACGCTCATTCTTAG
TGGGTTGCACAGGCTCCCTTAATACAGATGTGTTCTTATTCTCTGGTGCTCTCGCCAGGGGCACCGCTGCATCCATCTCTCATCTCTCCCAGTC
TGTTAGAAGGGTGTGTGTGTGTGTGTGTGTGTGTGTGTGTGTGATCTCTCTCCTGAACACTGGTGTGCATAGTGTGTACCTGTCACATAAGTGG
CATACAACAGAGTAAATAAGTTCAGTGTAGTTAAACACAGGACCTCGCCATGGCTCCAGGCTGTCCCCTGCCTCCCTTGCATTGTCCCCCACAG
CAGCCAGTGCTGTCACCATCCTGACACCGTAGGCATTACCTCATTCCTGTCCTCCTGGCATCAACCTCCCCTGCCTCCACATACTCCACATGCC
ACCACCGTGTGATCCGTAGAAATCTCATCCTGATCACATCATAGCTGTCATTAGTGACTCCTTCCCCTACTCTGGAGCACACTCCAGTCTCCCT
CACTGTCTCTCATCAGGCATGTGCAGACACATTAAACTCCACAGATGGCTCTGCCCTCCATCCGTGATGCCCATCCATCAGCACCCCCTTTCTG
GAGTGGCCTGATCTTCTTTCCACACTTGTCAGACTCCTCCTAGATGCTTTGTCACAGCCTAACACTGTTCTCACCCCCGCCCCACCCACTACAT
CCACCGTGACTGTCCACCCTGGGCCGGGAATGCCTGCACACAAAGCAGAGGTGTTTTGGAGAGATTGACATAAACCTCTTCTTTGGAACCTACC
TTAGTCTCGTTTAATTTCTCAAGAAAACACTTGGCTTGTAAAGTTCTGTTTATTCTACCTCATGCATTCTGACATATAACCATATATTGCCTTC
TGTTGTTATGTGAGTGTCTCCCATGCCTGTGGCCTGCTTTCACTGTCGATCCCTGGGTGTCTGATACCAGCGTCATCAGCTGCTCTTTGCTGCC
TAGCAAGATACCCCCAGACTCCGTGGCTTAGGACAGAGGCTATTTATTATTTTTTATGGTTTCTGTGGTCCACTGGACAGTTCTCGTTTCCACA
ACCTTCAGCTGCACTGAATCACCCGGGATAGATGTGGGTGAACAGTGACCCACAGCTGCCACCGCTTCGGTGTTTACAAACAAAAGAAAACAAA
CAAAAACGTACTGGAACTCAGCCACACCATACTGCCAGCACTGCCTTTACCTTGTGACAGCAGAGGCAAGTCAGTGTAGTGGAGATCACCACAT
GGCCTACATGTGTGCTGTTTGCTCCCTCAAGGAGACATTTACTAAACTAGAAAACCTTGCCGAGCCCTGCTCTGGGATGGCCAGGTGGAAAGCG
CCTTACCTGGGGCAGCTTGTCTTTCTCTGTGGTCTCTCAGCGCCAGGGGACTGGCCTGGTGGTTTGTATATAGCATAGCAGCAGAGCGCAAGGA
CCTGATATACCTCCATAAGCCTCTGCCTCCCAAGTGCTGGGGTTAAAGGCATGCGCCACCATTGTGTGCAGAAAATGTCCTTGACGTTGGCCTC
CCCAGATATTTCTAGGCCTGAAGACTGCTTCCCTACAAAGACGGGTCCTACCCAGATTATCTAAACCTGTTTGCTAGATCCAGCTGTATAGTAT
AAACCCTGCCTCCTTGCTTATCCGTGTGTAATAATTTCCACCTCTCTGCTGAGCTGTATGCAATAATTAGGAATCTCTGTCCAACTTAAAAGAC
CGACATGGTGAGCTTCCAGGGTGTTTTCATTTCTCCATCAGAGGGCCCAGACCACCTGAGGGCGGAAGAAACAACTTTTCTCAGTGTCCATGTG
TCAGCTGCCTCTCTTTTCCTCATTTCCTCACCTTCCCAGTCACGCCCATCCCTCGAACTCTGATAGCATGGCATGGCTCTTTGGGGAGAAGAAA
TTCAGGAATAGTATTGTGTAACTACTATGTGCCAGGAACAGCCTGTAGATGCGGTGATGGGCAGGGTACACCTACAGCCTGACTCGGCTTTCTG
TCGTTGTCATCCAGTTGGGAAATGAGAGTTCTCTAAAATCCTTTCAAGGAATCCTTCAGGAAGGAGCTATAGCATAGCTATATAACTCCATGGC
GATATGTGATGGCTGGTTGTCAACTTGATTAGATTAGTAAGCACATGAGAGACTAGTAAAGTGAACCTCTTTCTGCCTACATCTTTGGAGATGT
TTGCAGACATGGACCATGAGGTCTCTGACCTAGTCAGTGGTTTAGTCCATTGATACATTCTAAGTCTCAACAGATTAATGGAGGTTCTTGGCAC
TTCCTGGCTGCTGACAGGAACCACGCCATCTAGGAAATGCTCTATGGGACTGTCTTGCCCTGGTCCCTTTCTGAATTCCTCCTCTGTTTTCTGA
ATGCTGTCATATAACCTCTACCGCCATGCCCCTCTCCTCCATGATGAGTTAGAACTTCTGAACTCGGGAGCTAAAATGAACCTTTCCTGCTTTA
AGTCGTTTCTGTCAGGCATTTTGGTTTAGCTACAACTAGCTAACCCAGTCTGCCATATTCACCCTCCCCGACCTCACACAATTATCTGAGCTTT
TCAGATGCTGTGTCTTGAGGCTCTTTCTTGCTGGTCGCCTGACACATTTTCAACCAGATCACCGTTTCATCAGGACACCAGTAACTCGGAAGTC
TCATTCACTAGATTGACTCCCTCAGGTTTCTCCTGTGGTTGTAACGCTCTACTCCATGCATTCAGATTAATCCATGCCTGCCTTATTCCCCCTG
TGGATACACAATATTCCCCTTCTCCACATGTCTGGTGACATCCTACCCCTCTGATGAAGCAGCCTCAAGTACCACTCACCCGTGCTGGTTCCCT
GGTTGTTCCTCCAGCGAGCTCTTTCTTCCTCTCCTCTCACAGGCCTCGGAGTTTGGAGTGTGATGCAAATGGGGTGTAGCACACACTGCTGTTG
TCTTAGCGTTTAGACTGCACGCCAGGTCATGATTCACTTTAACGTCCTTCTGAATTCCTTTAACAGGGGAGACGTCTAGAAAAGTCTTGGCTTC
GTTGCTGAGGCTGGAGCTCAGGTTGCGGGAAGCAATCATGATCTGCTGATCTGTACCCCAGTTCCACTTTATGAATAATGGACAACTGATTGGC
ATGACTCCCATTTCCAAACCTTCGTCTTATTTCCAAGACTTCATTGGGTTAAATGTATTTCATACTCATTAATAAAGAAAAAATTGCTGCATAT
AAGCCATCAATTATATAGTGTATCTGAAACTATGTTTCCTAGCACTAGAATATAGATTATTAAGTCTAATTCAGATGGATATAGGTGACTCAAT
ATATTTTCAAAACATTCAAATATACCTGTCATTACAATCTCAAAACAGCTGTGGTACAAAGGTTGGTACATGGTAGTATCCTCATCTTACAAAG
AAAAAAAGTTTAACTTAAAAGAAACTATAATACTTGTACAGAATTAAGTAAATATATTTTTGTTTATATTAAATTAATAAATATATATATTATA
TACGATATATACTCAAAGTGTAGGCGTCTAGCCTTATGATTGAGACAAAGCTTCCTTTCTAAAATCACCTGTGGATATCAAAACCTCAGCTCTA
AGATTGGTATCAAAACTGATCTTATTTATAAATTGCTATGCACATCAACTGGACCTTCAGTTTGTCTTCCTGGCTACTCATCCCGGTATTACTT
GTATAACATAATGTGATGTGGTATTACATGCAGCTCAGGAGAGATTAGAATGATTATTGGGTACATATGTTTTCCCGGGGATGTAATTAGGCAG
ATTCTCACACCCTCATCAAGAGATTTCCCCACCATCATTAATCTCAAAATAATAGAAGGCTTTCTACATCCAACATTACTTGAAGATTTGTAGA
CGCATCCTCTAGAGTGGACATTTAGGGGGAAAAAAAGGCTTTGTGTGAAGTTTGGGTATTTGTTATACTTTAAAAACGAACATGCACACTTTGG
CATATGTAACTATTAGACTGATGTTCTGAGGATAGATCTGCATACCATTAGTTGTATTTTTGTTTTACATAGTTTGCTAGGTGGAAGGAAGGGG
CTTTCAAGTTGAGGAGGCTCTCCAAACTGCAGCATCTCGAATGTGTTGATATCTACAACAACCAATGTAAAGAAGAAAAGATTTGAGTTAGCAT
ATGGATGTGGCAAGTTTTCCTACATAGTCCCAGATTTCACTGATGCAGGAACCATGGTGATGCAGGGCATCATGGCAATGCAGCAGCCTGTAAC
AGAAACTTCCTACTTCATGCTACTCAGGAAGTGGGAGTGGAGATGGGATGCAGGAGTAAGAGACAGCACCTAAGAGCACACCTACAGTGATTCT
TCTTCTTGCACGCCCTATCTTCTACAGTCCCCCAACCTCCCAAAATAGTGCCACCTTATGGTTATAGTTACTTTTGTGTCTCTGTGCTGTGATA
CCATGACCAGGGCAACTGACAGAAGAAACAGTTTATTGAGGCATTTTATTTTCAGATAGTGAGCCCATGGCCATTATGGAGGGGAGCATGCCAA
CAGGCAGGCAAGCATAGTGCTGGGGCAATAGCTGAGAGCTTATATCCTGTTCTGTGAGGCAGCATGGGGCCAGAGACGGAGACACAGAGAGAAG
GAGGAAGGGGAAGAGGGAGAGAGGGAGGGAAGGAGGGGAGGAGGGGAGGAGGGAGGGAGGGAACCAACTGGAAATGGTGTTCTGAAACCTCAAA
GTCTGTGCCTCTGGTGACACATCTCCTCTAACAAAGCCACAACTTCCAGTCTTTCCAAAACAGGCCCAACAGCCTGAGCCCAAGCATTCAAATG
TATGAGCCTACGAGGGTCCTTCTCATTCAAACCACTACACTAACCTTCTTCTAAAACAAAACAAAACAAAAATCCCAGACTTTTGGGCCACATT
CACATATATTCTATGGCTTTACCACTGTTTGCTGTGATACAGGTAGTTTGAAGCTAAGTTATATTAGACCTCATGAGATTTAACTAACCGGTCC
TTGTATAATATACGTGTCCTGGCCTTTCTGTCTTGTAGATCATACGGATGACAGATTCTGTGTTACTGTCTTTTTACAATAATTTTTTTTCATT
TATTCTTTGATAACTTCATACATGAVTACAGTGTATCTTCCTTATAACCAATCAAAATCCTCCTTCCCACTCACTTCAGAATCCCCAAATACTT
CTTCTCCCTTTTACCTTCATGTTCTCTTTTTTATTTTTTTTTTTTTTATTTTTTTTTTATTTTTTTTTTTAGGATTTTATACATTGACTTGTGA
TTATCTTCACCTTCCCTACCCCAACTTTTCCTCGATCCAGCCCCGGTCTCTTCCCACTGACTTTGTTTCTTTTTTTCTCCACCAATGCCAATTT
GTGCTGCTCAAATATTCATGTATATATGGCCTTCCACTGGAGCATGATTAACTTATCAGGGGCTGCACTCTTAGAGAAAACTGACCCTGTTGTT
TTGTTGATTTTTTTCAATAATGTTTTTCTTTCTTATTACTCTCCTGTTTAACTATTATTCTAGCATGGTTACTTGGTCCTGTCATCTCAGGGAT
AAATTTTTTTTTTTCTATTTAGTTTGAAGCATTCCTTCACATGTCTACTGTGGAGCTGACAATGGTTATACATTTCTTTCATCTGTTTTCACTA
TGGAAAGTTTATATTTTCTATCATATTTTCCTCCTGATAGACAGCTTTGCTGGGTATAATAATCTAGGTTATATCTTTCAGGGCTCAAAAAACA
CCATCCCACGCCTTTCTGAATTTTAGAGTCTTGGTTGAATGGCAGGCTGTAATTCTGATGGCCTGAGTTCATGTAAGACTTAGCATTTCTTGTC
ACTTTCAATATACTTCCTTTGTTCTACATACTTACTGTCTTAATTAAAATAAGACTAAGAAGGTTCTTTTCTGATTTTCTCTGTTTGGTCCTAA
ATGCCTCCCATATCAGGTTGACATCTTTTCCTAAAAAGGGGAAATTTTCAATTATGATTCTGTTGAAAACATTTTTATGTCTTTAGAACAAGAT
TATTCTTCTATGCCTATGACTGTAGGCTCAGTATTTTCAGAGTGCTGCACATACCGTGAAACTCTTTGACGTCTGTTCTTATCTAATTGTGCCA
TCCCTCCACTTTCTTTTCCAGCTCAGAAAATCTGTGATTTTCCTTGACCCATTTTATTAGTGAGACTTTCCACAGAACTTTTTATTGGTTATGT
TTTTAATTTCCATCATTTCAAGAAGTTTTCCATCACTGTCTTTGTTTTCATAGTTCCTCTTCCGTATCTTCCACTGTGGTCTTTATTGCACACA
GTTGGTCATTTGTGTGCTTAGTCAGGAGCTTATTTCCTTTCATTTCTTGAACAATAATTCTTAACCCTGTCTAGGGTTTCATCTGAATCACTCT
CACTAGACGCTGTTACTCAAAAGTTAGTTGTTCTGGAGAAGTTATAGTGACATGATTTTTGTGTTTTTTCTTATTACTATTTTGGGAGTCACAT
ATCTGGCATTATCTTGTTGCTTGCGGTTTTTTCTTGCATTGGATATATTCTTTCATTTGAAGTGTTTGCAGAACTCAATGGGTGCTTGAATTGG
TAACGTGGGGTATGTTGTTTAGAAATTACTTAAGTCCGCGAACTAGGCGCTGCTACAACTTATATCTCATCTCTGTGTAGACTATTAACTGCAA
GAACAACCACGGATTCTGGGTATTGCCTCTAGATGTAAATGTTGTATTATCCAAATAACAAAAGCAATTCCTTTAACTTTAATAACTTTTGGAA
AATGAACAACCAAAGATAATATGAAGTATAGTTTGATTTTAGTTGATAAGGTTGGAATTGAGAGGGAAGGAGAGTGGGTGAGGCTCAAGACAAG
ATCAAAGGACAGTGGCAGAAATGTCGAGGTGATGCAGTAGCTGACAAGCCACCGTTGAAACAGTACCAGGGTCCACAGATATTAGAGATTGATA
AAGCTACGAAAGGGCCAGCCTGAGCTACAGAACAAGTCCTACAACAGTCACGACTACACAGACAAACCCTGTCTCAAAAAACCAACATAAAAGA
ACCATGGAAGTCTTTAATTAAGTGGTACAAAGAGGAGAACAAATGACAGAAAGAAGGAAAGTTGTCAAAATAAAAGGAATAAAATGAGATACAA
GTGAAAGGCATGGTAGTGTCTCATGTTAGCCTGAGTAAAGAAACACATAGCAATCTTAAAGTCTAACTGAGCAATATACCACAAATCCAAAACC
ATCATAAGATGTATATGCAAGGGTATAAAGGGTAATAAATAATATAAAATGAGCTGGGCAGTGGTGGCACACACCTTTAATCCCAGCACTCGGG
AGGCAGAGGCAGGTGGATTTCTGAGTTTGAGGCCAGCCTGGTCTACAAAGTGAGTTCCAGGACAGCCAGGGCTACACAGAGAAACCCTGTCTCA
AAAAAACCAAGAGAGAGGGGGGTGGAGAGAGACAGAGAAGCAGGGGATAGGGTGCTGGAGCAGTGGTTAAGAGCACTGGCTGTTCTTCCAAAAG
ACGCGCCTTCAAGTCAAGCACCCACATGGCAGCTCACAACCATCTATAACACCAGTACCAGGAGATCCAGTGACTTCTTCTGGCCTGTATGGCC
ACCAGGCACATGCAAGCTGCATAGACATGCATGGAGCCAAATCATCCATGCACATAAACTAAAAATAAATTGTAAGGTCCAAACGGGTCAAGGA
TGCTGGGAGAAAACAGCTCGCAGAATCAACTAAGCAGGCCTTATAGGAGCTCACAGAGACTGAAGGCCCAATCACGGTGCCTGCATGGGTCTCT
GCTAAGTCCTCTGCAGATGTTATGATTGTTTGCTTCGTGTTTTGTGGAGTAACACTGGGAGGCCGTCTCTCTGACTATTTTGCCTGCTCTCCAA
ACCCTTCTCCTTCTACTGAGTTGCCTCATCCAGCCTTACTATGAGGGTTTGCGCCTAGTTTTATTGCAACTTGTTATCCTGCATTCGGTTCATA
TCCCTGGGAGCCCTGGTTGGTTGGTTGGTTGGTTGGTTGCTTGGTTTTAAAGAAAGTGGATAAGAAGTGGATGTTGTGGGGGGAGTAGAGAGGA
GAGGGAGATGACAGGAGTGGAGGGCGAGGAAACTGCAGTTGACATGTATTATATGAGAGAATGAATAAATCAATGAATGATCTTTGGGGGGGGG
GGTTCGACACAGGGTTTCTCTGTGTAGCTCTGGCTGCCCTGGAGCTTACTCTGTAGACCAGGCTGGCCTCGAACTCAGAAATCCGCCTGCCTCT
GCCTCCCCAAAGTGAAAAGTAAAACTAAAAGTAAAAGAATACGTATGGGGTGAGTTGCAGGTACACCTGTGCTGAGTTCAGAGGTGAAGTTCTT
AGTCAAAGTAGCTCTGGCAAGTTGCAAATGCTGCAGTGTGGGCAGAGGAACCTGTGAGCCATTAATCTCTGTGACTTCTGTGTTCTCTGTGGTT
CTGTTATTGGGGAAGGGGAGGCTGTAAGTCCTTGCCTGGTCGCTTGGCACTCCTCTGTGGGCTCTGTTGGTCCTACAGGCTGATGTGTGTGAGG
AGCAGAATTTTCCCTCCAGTTTCTCCCGCTCTCTCTAAGCAGCTGAATTCAGCAGTGTTGTGCCAATCAAGAGGGCTCGAAGATGGGTCTCCAT
GGGGCACTTCTTAAGGGCTCGAATGTGTGGCACTTTCAAGAATGTTGGCACTGAAAATACCCGGTGTCTCTTCTACATAGTCACTTTTTCTCTT
TGTTAAGTCTTTCCCATTTTCATAAAAAATCTCTTTTTTTTTCTTGATATTATACTATATTACAACATTCCTCCATTCCTTTTTCTCCCTCTGA
ATCGTCCCATATACCTCTTCCAAATCTTTCTTTAAATTCATGATCTCTTCTTCACTAATTTATTATTGCATATATGTATACATATACATATATT
TCTAAATATACACATTACTAAATGTAACCTGTGTGGTCTATCTAATGTTACTTGTATGTGTATTTTCAGGCCTGACTTTAGTCTCCCACTCCTA
ATGTTCTCACTTCCCCATAGTTTGTTCTGTAGGGCTGAGGCTTGTGCGCTTTTCCCGGTCCATCTTAGCATCTCCATTGGTACTACCCTTGTTC
GGTTCATGGTTAGGCAGACCTGTTGGGGAGACGAGATGAGTATAGCTTCTGACATTGCTAGGAGACATAACTTCTGCCACCTTCTCTCGCCACC
ATGATACTCTACCTTACTCAGACTACAAATGGTAGTTCTCAAAATTATGCACTGAAACCATATGCCAAAATATCTATCTCTTTAAATTATTCCT
CCCAGGAGTATTCCACAGTCTGATTATCACACTAATTTATTTTTATTTATATCCTGAAGTGCTGCCTTCCCCCTCCTTCGCCCTGTGTTTTCAT
CCTTTTCTCCCAGATTCACTCTGTGTACAGCCACTAATACGAGTTTTTCCTAAGTAGTGATCAAGTGTCACTCCCATGAGTGCTCAGTGTCCTA
AACTGGCTGTACCCTACGGTTCCAACTGATGCAAAGAAAATCTCATCTTCTAGGATACTTCACTGGTTACAACCTTTACACTCTTAAAGATCAC
CAAGTATGGAAACAAAATGCCATAAATCAAAACCAGCAAAAACAACAAATGATATATATGTTCATTATCCCCTACCTACCAAGGATGCTATATG
CTTGAATTAACAAATTAAGAAATAAGAATAGTTCTGCATTTAAAATTTAAGTTTAAGATGGAATATTAAAAAAATAAACAGGAAGCAATGAGCT
ATCAGATCACCAGCCATCTTTCAATATCAAAGAGAAATGTTCTAATACTGAAAACAGTAATTATCGATAACTACTGTGGATGCCCTCACTTGTA
ATGAGTAAATTAGTAAAATGGAAGATTAAACTACAACAGATTTATACAGTGACACACAAACAGACAAATGAAGCTAAAGAAAAAAGCTCCTAGA
TATATCAGGCATCGTCTAAAGCCTGGTTGAGCCCACCCATCTCCCAGATACAACGTGAAGCAGAGTGCTCTCAGTGGTGGTCCATACTGCTGCT
GGAGAGTTCCCTAAGGTGAGAAATGTTTATTGCCTTTACACTCAGCCTGAAGACCCTCAAATGCTACATTTCAGAAAGACTAGGGGAGAGTAGA
GAAATGGCTGAAAANNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNAGTAAGAATAATACCTTAAACTTCCAAAAGCATTGTTTTAGATTTATTTTAAGTGTAAGAGTGTTTTGTCTGTCCTGTCTGGTGTATG
TATGTGCACTACATGCATGCCTCTGACCTGGTGCTGGCAGAGGCCAGAAGTGGCCACTGGATCCTGTGAAACTAGACTTCCAGATGCTGTGAGT
CCAGTCCTCTGCAAAGTCAACACTGCTGTTAACCACAGTTGTCTCGCCAGCTCCTTGAGACATACTTTTAAAGTTAACAATGGAAGAAGATGTA
AAATCATTTTTTAAAAAATCAGAGGAAATAAAAAGCAGAAGTACATTTGTAGAAAACAAATGTGATTATATTAACAATCCCAAAATATATAAAT
GACATCCATAAATCACAACATACATAAATTATAAAAGAAATGAATAGATTTCTAACTAAGACAAATGCATTGAGATTAGCGGGTTTTGGTTTTT
TGTTTGCTTTGTGTTTTTTTTTTTTTCCTCAGAAAATTTCGTTGACTGTAGTGTCAGGTGGTGATGAGCTGAACAAGAAGTAGTCAGAATTTCT
TAGCTAGCAGTAGTATCTTAGCTGTGTGTCTTGGGGACAGGTTTTTCTAACATGCACTGAGAAAAGTCATAAGTAAAGGGGTGCAAATCATTCC
AGCAAAGACTAATCAAAAAGAATCTAGAATTGTGTTATTTTTTAAGTCATCAAAATAATTTATAATAGTTAAATGCCTCTTAAATACACATGAT
ATCTTCTGAAGCTAAAAGTAATATGCACTCAACCAGTTTCTTAAATCTATTTGGAAACATTAAACATGATAAAAGTAAAAAAAAAAAATCTCTT
ATGAAGTCCTCTATGAAAGGAAATTGTGACAGGTTCCTGATTAGACAGAAACCATTCCATCTCCAAGGGAGAATACTCAGTGTAACTGTGGCTT
CCTAGAATGAATTGGGTAGTGTTCATTCTGTTTCTATTTTGTGGAATAGTTTGAAGAGTATTGGTAGTAGGTCTTCTTTGAAGGCCTGTTAGAA
TTCTACACTAAACCCATTTGGTCCTGGGCTTTGTGTGCTTGGGAAACTTAATCACTGCTTGTATTTCTTTAAGCATTATGGGACAGTTTAGATG
GCTTATCTGATCCTGATTTAACTTTGTTATTTGGTATCTGTCTAGAAAATTGTCCATTTCATCCAGATTTTTCAGTTTTATTGAGTATAAGCTT
TTGTAGTAGGATCTGAGGATTTTTTGAATTTCCTAGTTTCTGTTGTTACATCTCCCTTTTCATTTCTAAATGTATTAATTTGGATACTGTCTCT
GTGGCCTCTGGTTAGTCTGAGTAAGGGTTCATCTAGCTTGTTGATTTTTCTCAAAGAACCAGCTCCTGGTTTTGTTGATTCTTTATATAGTTCT
TTTCATTTCTATTTGGTTGACTTCAGCCCCCAGTATGATTATTTTCTGCTGTCTTCGGTGTATTTGCTTCTTTTTGTTCTAGAGCTTTCAGGTG
TGCTGTCAGGCTGCTAGTGTGTACTCTCTCCAGTTTTTTTTTTTTTTTTTTTTTTTTTTCGACGCACTCAGTGCTGAGTTTTCCTCTTACCACT
GCTTTCATTGTTTCCCATAACTTTGGGTATGTTGTGCCTTCATTTTCATTAAATTCTACGAAGTCTTTAATTTCTTTATTTCTTCCTTGTCCAA
GTTAACATTAAGTAGAGTGTTGTTCAGCTTTCATGTGTATGTGGGTTTTCTGTTGTTTTTCTTGTTATCCAAGACTGGCTTTACTCCGTGGTCA
TCTGACAGGATGCATGCCATTATTTCAGTCTTCTTGTATCTGTTCAGGCCTGTTTTGTGACCAGTAATATGGTCAGTTTTGGAGAAGGTACCAT
GAGGTGCTGAGAAGAAGGTATATCCTTTTTTTTTTTTAGGATAAAATGTTCTATAGATATCTGTTAAATCCATTTTGTCCATAACTTATGTTAG
TTTCACTGTGTCTTTGTTTAGTTTCTGTTTCCATGATCTGTTCATTGCTGAGGGTGCCTGTTGAAGTCTCCAATTGTTATTACTGTGTGAGGTA
CAATGTGTGCTTTGAGCTTCAGTAAAGTTCCTTTTATGAATGTGTGTGCCCTTCCATTTGGAGCACAGATGTTCAGAATTGAGAGTTCTCCTTG
GTAGATTTCTCCATTGATGAGTATGAAGTGTCCTTCCTTACCTTTTTGATAACTTTTGGTTGAAAGTTGCTTTTATTCGATATTAGAATAGCTG
CTCCAGCTTGTTTCTTCAGACCGTTTGCTTGGAAAATTGTTGTCCAATGTTTTATTCTGAGGTAATGCCTGTTTTTGTCACTGAGGTGCATTTC
CTGTATGAAGCAAAATGTTGGGTCCTGTTTATGTATCCAGTCTGTTATTCTATTTCTTTTTATTGGAGAATTAAATCCACTGATATTAAGAAAT
ATTAAAGAATAGTTATTGTTGCTTCCTCTTATTTGTGATGTTATTTTTATATTTCTGTGGTTATCTTCTTTTGGGTTTGTTAAAAGAAGATTAC
TTTCTAAGTTTTTCTAGGGTGTAGTTTCCCTCCTTGTGTTCGTGTTTTCCCTTTATTATCCTTTGTAGGGTTGGATTTCTGGAAAGATATTGTG
TCCATTTGTTTTTGTCATTGAATATCTTAGTTTCTCCATCTATGGTAATTGAGAGTTTTGCTGCGTATAGTAGCCTGGGCTGACATTTGTGTTC
TCTTAGGGTTTGTATGACATCTTCCCAGGATCTTCTAGCTTTCATAGTCTCTGGTGAGAAGTCTGATAGGACTGCCTTTATATGTTACTTGACC
TTTTTCCCTTACTGCTTTTAATATTCTTTCTTTCTTTTGTGCATTTGGTGTTTTGATTATTATGTGTCAGGAGGAATTTCTTTTCTGGTCCACT
CTATTTGGAATTCTCTAGGCCTCTTATATGTTTATGGGTATCTCTTTCTTTAGGTTACAGAAGTTTTCTTCTATAATTTTGCTGAAGATATTTA
CTGGCCCTTTAAGTTAGGAATCTTCACTCTATTCTATACCTATTATCCTTAGGTTTACTCTTCTCATTTTGTCCTGGATTTTCTGGATGTTTTG
GGTTAGGACCTTTTTGCATTTTGCATTTTCTTTGACTGTTGTGTCAATGTTTTCTATCGTATCTTCTGGACCTGAGATTCTCTCTTCTATCTCT
TATATTCTGTTCGTCATGTTTGCATCAATGACTCCTGATCTCTTTCCTAGGTTTTCTAACTCCAGGGTTGTCTCCCTTTGTGATAGTTTTGTTC
AATTCCTTCACCTGTTTGGTTGTTTTCCTGCAATTTTTTAAGATATTTTTGTCTTTCCTCTTTAAGGGCTTCAAGCTGTTTATCTGTATTCTCC
TATACTTCTTTAAAGGAGTTATTTATGTCTTTGTTAAAGTCCTCTCTCATCATCATGAGATATGATTTTAAATCAGAGTCCTGCTTTTCTGAGG
TATCCACGCCTCACTGTGGTGGCAGAACTGGGTTCTGATAATGCCAAGTAGCCTTCGTTTCTGTTGCTTATGTTATTGCCCTTGCCTTTAGACA
TCTGGTTATCTCTCGTGTTAGCTGGTCTTGCTGTCTCTGACTGTGGCTTGTCCTTCCTGCAAGCCTGTCTATCAGAACTCCTGCGACACCAGTT
CTCTCTGGGAGGAATTTGGGTATGGACAGCTGTGGTACAAGGTCAGCTCTGGGGTGCAGACAGAAACTGCAAGGATCCTGTCCCCAGCTGCTCC
TTGCTTCCTATGTCCTGAACCCTCTGGGCAGGTCCCAGGAATTTGAGCAGAAGTGGTGGTCTTACCTGTGCTCACAGGCATGTCCGAACTCCTG
GGAGACTAGCTCTTTCCCAAGGGTATATAGATATGGAGTGCCATGGCACATGATCTAGAATTCCTTTATTGATACGAGTCAAAATACACTCAAA
GACAAAAGGCGTTGTAAGGCTAGAACTCTTACATATGGCTTGAAATATATAAAATGTTAAAACTACACATGACATTGGAAATCTATACTATTTC
TAAAATGGCCTCAATATATGTAAAACAAAAATTAATAAAACTTTAGTGAAATTTCACATATTTCTTTAACGTATCACTTGATGGCGCAACAAGA
AGTAATAAACATGGAATTTAAAATGGTAGCAGAAGACATTTTTTTTTCATAGACACAGGAAACTGCACCAAGCACCTAGAATATGCAGATCCAC
TCTGCATGGAGGAATTATTAAAAAAAAAAAAAAAAAGTTGACCTCACACTAAATGCAAAACTGGTCTCAACAGATTTCCACGAATCACAATTAA
GATAATCATATATTTTTTAAAGATTTATTTATTTACATATATGAGTACACTGTAGCTGTACACATGGTTCTGACCCTTCATGTGGTTGTTGGGC
TCCAGTCAACCCCAATTGCTCTGATCAGCCCCCCTTGCTCCAACCCAAAGATTTATTTATTATTATAAATAAGTACACTGTAGTTGTCTTCAGA
TGCACCAGAACAGGGCATCAAGTCTCATTACGGATGGTTGTGAGCCACCATATAGTGCTGGGATTTGAACTCAGGACCTTCAGACGAGTAGTCA
GTGCTCTTACCTGCTGAGCCATCTATATTTTATGACAATTAAGTTACCATTCAATAACCATACAGTCAGCCCCCTCCCAAGTTCCCCTTAAATG
TGGAAATTTTCAAACATTTCTGAGTAAATCACTAAAACCGAAATTTTTAAATACTTAGAACTCAATCGTCATGAAAATAGTACATTTCAGAACT
TGAACGATGCAGCAGAAGTTAGATGTGATAAGCTGTGGGGCAGTAGAGGGCAGGATGAGGCGAAAACAGTAGGCAGGAGTGACATTGGCAGTAA
AGATGCTGTCTAGTGAATCTACACATCCCAGAAGATGCTTCTGTGGATAGGCTCATGGTACAGGGATCAACCAGACTTTTTTTTTTTTGTAACA
AGATCATATGTCTGGCTTTGTAGGTGACACTGTCACCACTGCTCAGCCTTGCCACCACAGTATGAAAGCAGCCATGTGATGTATAAATGACTGA
GTATCGTTCTGGTCTAATAGTTCCATATATAAACTAGCAATCGACCATACTTACTATTCAGACTATGATTTCCCAGTTCTTGAACTAGTTATAT
GTCTAAAACTGACCGTAAATGATGGGAAATTTGAATAAGATTGCTGGATATAAGATCAGTATACAAAAACAATTGCCTTTTCATTTGTCAGTAT
CATGGAAAAGTAACTTTTTAAGAAGTGGTCACTGAGCCAGTGGTACTTGGCTATAATTTTACCACTGCAGAGGCAAGATGGTCATGTGATTTAA
AAACTAGCCTAGACTACATAAAAAGAATCTACCTTAAAAAATGCTTAGGGTTGATAGTCACAACAAAAATAAGTAATAACTGTACTAATGTTCA
GAGTCTAAAGAAACTAAAACAATTAATTGATCTGCTTCAAACCAGTAGTTCTCAAAAGTTTGATTTCCAGATCCTTCTATGCTCTAAAAACAGC
TGAGAAGTCATAAGATCTCTTACTTAATTGGGCTTTATCAATCCGTAGTCATAAGACATTTAAGTCAGCTACTAATTTTTAAAAATAATGAATC
TGCTATACACTAAATGTTTGACACATCTTTATGAAGTAAATTGCACTTTCAATTTAATTGATGAAAATGTTTTTATATTTTTTCCAGCTCTCTT
TAATGACAGCATAAATAAAGGCACTGGAGTCCTGAAGCTCTTTCAATGTTGAATCTGCTGTGCTCTGTAGTTTTACTTTTATTATATGAAAAAA
CCTATGTCTTGACCTGCAGCTAGAGAAAGCGACCACACTTTAATAGCTCCCCAGTTTCCAAACAATGGAAGGTATCTCTTCTTTGATACTTTAT
CAGTCTTTGGCAGGTCATAACTAAGAAGGTAGTTGCAATATGGGACCTAAAACTGTATCGCTGAGCTTGTCATGTTCCATCATATTAAAATTCA
TTGTTCTGCCTGACACCCTTGGATGCATCTTTTATCCAAGGATGGTTGTGTGAAGGCAGTTGACTATTTGGAAAAGGCTGGGTTGCCGAGTTAA
GTACCTCTTCAAAATGATGATACTGTTTTATGTCAAATTTTTCACTTATTAACATGATCTTCACGCGGAAAAAAGCTTTCAAGTTCCGACAAAC
TGTCAAGCTAACAGTAGCAAATGTACACTTACCAAAACTCTGAATTTCATTTGAAAGCTTAAATTTTATCATTGGCAGCAAATACTGTCACCTG
TTTTTCCTGAAGTCACAAATCTCACTCACCTTGTTCACTGTCAACAAAGTATCTGCCAAATCCCCACGTCTGGATAACCTCAGTTTGTCAGCCG
TTCTTCCTAGTAAAAAAAAAAAAAAAAAAAAAATCACATTATGTGGGAGAAAATGTGTCCTAAGCATGTTAAACTTACAATTTGCCTGAGCACA
GTGGCATCCTCCTTTAAGCCCTTTAATTCTAGCAGAGGCAATGGGAACTTGTGAGTTCAACCCAGCCTGGTCTACATAGTTTCAGGCTACCCAA
GTTACATAGTGAGACCTTGTCTCAAAATTAATTAATTAATAATTAATTATTAGTAAAAATATTAAGATAAACTTACAGTTCAAACCACCCCACA
AATGCTCTTCCATGAGCTAACAGTCATATTTCAATGTGCAGTGATACTGTACATTGCACACAACATTAAGATGTTAAGTACTCGAGTGTCTTCA
GAGAAATAACAGTTTCATGGATGAACACGGACATTTTTTATTAAACTAATAAAATTTATTTAAAAATACACAACTTAGCATTAACATTTTTAAG
TCATCAAAATAATTTATAATAGTTAAATGCCTCTTAAATGCATGTGACATCTTCTGAAACTAAAAGTAATATGCACTCAACCACTTTTTAGAAT
CTGTTTGTAACATTAAACATGATCGAGGTAGAAAAAAAAATCTCTTATGAAGTCCTCTAAGAAAGGAAATTGTGACAGGTTCCTGATTAGACAG
AAATCATTCCATCTCCAAGGGAAAATATGCAGTGTAACTGTGGCTTCATAGACTATATTGGGTAGTGTTCATTCTGTTTCTATTTTGTGGAATA
GTTTGAAGAGTATTGGTAGTAGGTCTTTTTTGAAGGTCTGATAGAATTCTCCACCAAATCCATCTGATCCTGCGCTCTTTTTGATTGGGAGACT
TTTAATGACTGCTTCTATTTCTTTAGTGGTTATCGCACTGTTTAGATAGTTTCTCTGATCCTGTTTTAACTTTGGCACATGGTATCTGTCTAGA
AAATTGTCAATTTCATCCAGATTTTCCAGTTTTGTTGAGTATAAGCTTTAGTAGGATATGAGGATTTTTTTAATTTCCTCAGTTTCTGTTGTTG
TGTCTCCCTTTTCATTTCTAATTTTGTTAATTCGGATAATGTCTCTGTGTCCTCTAGTTCGTCTGCCTAAGGGTTTATCTATCTTCTTGATTTT
TCTCAAAGAACCAGCTCCTAGTTTTGTTAATTCTTTGTATAGTTCTTTTTGTTTCTGTTTGGTTGATTTCAGCCCTGAGTTTGATTATTTCCTT
CCATCTACTCCTTGAGTGTATTTACTTCTTTTTTTTTTTCTAGAGCTTTCAGGTGTGCTGTCAACCTGCTTCAGCATGCTCTCTCCGACACACT
CGGAGCTATGGGTTTTCCTCTTAGGACTGCTCTCATTGTGTCCCATAAGTTTTGGTATAATGTGTTTTCGTTTTCATTAAATTCTAAAAGTTTT
TAATTTCTTTATTTCTTCATTGAGCAAGTTATCATTGAGTACAGCGTTGTTCAGCTTCCATGTGTATGTGTGCTTTCCCTTGTTTTCTTGGTAT
TGAAGACCGGCTTTACTCCATGGTGATCTCATAGGATGCATGGGATTATTTCAATCTTCTTGTATCTGTTGAGGCCTGTTTTGTAACCAATAAT
ATGGTCAGTTTTGCAGAAGGTACCATGAGGTGCTGCGAAGAAGGTACATTCATTTTGCTTTAGGATGAAATGTTCTATAAATAATCTGCTAAAC
CCTTTAGCTTCTTAACTTCTATTAGTTTCACTGTGTCTCTGTTTAGTTTCTGTTTTCATAATCTGTCCATTGCTGAGAGTGGCGTCTTCAAGTC
TCCTACTATTATTCTGTCGGCTGCAATGTGTGCTTTGAGCTTAAGTTTCTCTTATGCATCTGTGTTTCCTTGCATTTGGAGCACAGATCTTCAG
AATTGAGAGTTCATTTTCGTAGATTTTTCCTTTGACTAGTATGAAGTGTCCTTCCTTATCTTTTTTGATAACTTTTGGTTGAAAGTTGTTTTTA
TTAGATACTACAATGGCTACTCCACCTTGTTTCTTGGGACCATTTGCTTGGAAAACTCTTGTCCAATGTTTTACTCTGAGGTAGTGTCTGCCTT
TCTCACTCAGGTGCATTTCCTATATGAAGCAAAGTGCTGGATCCAGTTTACATATCCAGTTGGTTAGTCTATGTCTTTTGTGGGGGAATTGAGC
CCATTGATATTAAGAGATATTAGAGAAAAGTAATTGTTACTGCCTCTTATTTTTGTTGTTAGAGGTGGAATTATGTTTGTGTGGCTTTCTTCTT
TTGGGTTTGTTGAAAGATTACTTTCTTGCTTTTTCTAAAGTGTAGTTTCCCTCCTTGTGTTGGTGTTTTACAACTTTGTAGGGCTGGGTTTATG
GAAAGAGATTGTGTAAATTTGGTTTTGTCATGGAATATCTTGGTTTCTCCATCTATGGTAATTGAGAGTTGTGCTGGGTATAGTAGCCTGGGCT
GGCATTTGTGTTCTCTTAGGGTCTGTATAATCTCCCCAGGATCTTCTACCTTTTATAGTCTCTGGTGATGAGTGTAGTGTAATTCTGAGAGCAT
TGCCTTTATATTGACCTTTTCCCCTTACTGTTTTTAATATTCTTTCTTTGTTTTGTGCATTTGGTGTTTTGATTATTATGTGTCCGGAGGAATT
TCTTTTCAGGTTTAGTCTATTTGGAATTCTGTAGGCGTCTTGTATGTTCATGGGCATCTCTTTCTTTAGGTTAGAGAGGTTTTCTTCTATAATT
TTGCTGAAGATATTTACCGGCCCTTTAAGTTGGCAATGTTTGCTCTCTTCTATACCTTTTAACCTTAGGTTTCATTTTCTCATTGTGTCATGGA
TTTCCTGGATGTTTTTGGGTTAGGAGAGTTTTGCTTTTTGTGTTTTGACTGTTGTGTCAATGTTTTCTATGGTATCTTCTGCACCTGAGATTCT
CTCTTCTATTTCTTGTATTCTGTTGGTGATGCTTGCATCTATCGCTCCTGATCTCTTTCCTAGGTTCTCTATCTCCAGGGTTGTCTCCCTTTGT
GATTTCTTTATTGTTTCTATTTCCATTTTTAGATGCTGAATGGTTTTGTTCCATTCTTTCACCTGTTTGGTTATGTTTTCCTGCAATTCTCTAA
GAGATTTTTGTGTTTCCCCTTTAAGGGCTTCAAGCTCTTTTACCTATGCTCTCCTGTATTTCTTTAAGGGAGTTATTTATGTCCTTCTTAAAGA
CCTATATCATCATCATAAGAAATGATTTTTAGATCCAGATCTTGCTTTTCCAGTGTGATGTTGTTCCAGGACTTCCTATGGTGGAAGAATTGGA
TTCTGATAATGCCACGTAACCTTGGTTTCTGTTCATTGTGTCCTCATGCTTACCTCCTGCCATCTGCTTGTTGCTAGTGCTACCTTCCCTCATT
ATATCTGACTGGAGCCTCTCCTTCCTGTGATCCTGGTTGTGTCAGAACTCCTCAGAGTCCAGCTTTCTCTGGCATCCTGTCATTCTGGCATCCT
GGGATCCTGGGATCCTGGCATCCTGAGATCCTGCGTATGTCAGAGCTCCCATTAGTCAAACTACCTCTGGGACCCTGATATCCTGGTGTGGCCA
AGCTCCTCCGATCATGTCATCCTGCGATCTTGGTCATGTTAGAGGCCCTGAGAGTGGAGCTTCCTCTGGGTGTTGTACGAGTCGCTATGGAGTT
TGCGACCAATGTCTGAACAAGTACATTTTAAAGTAAAACTCATGTCTTTATGTTTTGCTGATGAGTGTGTCCTCGTAAAGAACTCACACAGTTT
GGTGCCATACCAGCTTCCTGCTGACGCACCAGACTTTTACCAACCATTGTGTTTGCAGCCTTGGTGGAAATGCCAATGCTGTGGAATAGTCATT
AAGCTTCTTTGTATTGCAAGGTGAGCATCAGTACTGTGAAAAATAAACTACAAAATTCAAACCCTTTGAGACACTGACATGATAACACAAGAAC
AAAAGTCCATACCAAGTCCAAAGTCCCAGTCACAGTCTAGAAGCACAGGGGATATTGTACGGAATGAAGCCAGGTGTCAGGAGCCATGGCTGGG
CGGTTATGAGCACTAGCTGCTCTTCCACAGGTTCCTGGTTTAATTCCCAGCACCCAAATGTTGATCACAACTCCCCATAACTCTAGTTCCAGGG
CATATGATGCCCATGTCTCACCTCCTCGAGCACCTGAAACACACATGGTGCACATACATACATGCATACAGTCCAAATACTCATACATGTAAAA
ATAATTTACCTTCAGGTTATATATGCATGAGGTATATATGAGACATAAATTATCTTTCTTCAGACTTAGGTCCTGTCCCAAGACAGCTCATTAT
GTACTTGCAAGTATTCCAAACTCTCAAGCAGTCTAAAATCTCAAATTCTCTTGGTCCCTAGGATTTTGCATAAGTGATACTTGACATGTAGTAT
CAAAATAGTATTCGTTTAACTCGCATTCTGAACAAGCAAACAATCCTACAGATACACATACATCCTTTGTCAGAAAGTCCTGTGTTTCTTCCTA
TGTGTCACTTGGTTCTACACGAATGTTTAGACAGGCGTCATCTCTAGTCCTCCGCTGACTTTCTTTTGTGTCACCCTATTCTGTCTTTATGGTA
GAACTTGGGACTATTGAGTAGTTTACTGTTCATTTGCTTGCGTGTTTATTGTCTGTCTCAAGACCTGACATATGACTCTGTCCTAGTCAGCACA
GTAACTGTTTCTGAAACCTAAAGCACTCGGTAAGTGTCCATGGGTTAGTTAAGTCAGGTTGAAGTGACACATAAGGATCAGCTCTTCAGCATTT
CACTTTCGGAGAGGTTCATGGCTACAAATAGCTAGTTTTACTCATTCTGGCTTCTCTAGATAACAACACAGGGAACCTTTGATCTCAGTGCCTT
CCAGCATTGACTCTGTCCCACCAGGATCCTCGAGTTCCATATTATTTAACGGGGGTTACAGAAAAAGCACCTGACCGACAACCTCTGTTTCCAG
TATGAAGAGCTAGTGAGTGTTCTGTTCTAGAACTGAATTCACACCCTTCAGCTTCCTCCCAGGCGAAATAGGGAAGAGATGCATGCATACCGAG
CCACTACCAATGACAGCTCTCGGTGCCAAAGTTGCAGCAGCTCCCTGCCAAATGATCAGGCAGTAGTCATTCAGAAAGTGTTTGTTACAGGGAT
TTTTTTTTCAGAATTAAAATTCAAACTGAAGTTGCAACAAATGAGCAGCAGATTTTTCAGAATTAATTTTTTGTACATTTTAGAAACGTTTGCT
TCAGTCTTCAATTCCCGAGAAACCATTGTATCTATTCAGTCTTTCGCACAGAAGTTCTCAGGATCTTTCCTAAGAACTGGCAATGAGATTCATA
GTTCATTCCCCCCAAAAAATGTATGTTTATGCTTGAGAACTTGTGACTGATTATCGTCTCCGTTTAAAGACAGTGGACCAAACCCTCGTCTCAG
CTTTTCTCCCCTTAACTTTATAGCATAGAAAATCTGTAGTGGGATGCCTGGGAGTCAGGTGGAAGCCAACACTAGAATGTGGGATGTCCATCCA
ACCACTCACTGCCTAGGGGAAGCTGTGTTTGTTTCTGGTGTTCAGTATTTGAAATCTTTAACAGCAAGAATAGATGTAGACAGCCACTCTAGGA
TTCTGTGTTCACCCTGAGTTCTCTAACTCAAACTCAACGTCAGTGCCCAGCCAAGTTTGACTTCTGACCGCATTTCTAGGGAAACTCAGAGCTA
GCTTACCTACAAGAGATAACGGTGTCATTTCTGAAAGTTACACTGCTGTCTATTTCCACACGTATCATTTAGCTTAACTTGGCTTTAGTTACAC
TGTAAACTTGCCTGGGAATGTCAAAAGTGGTGAGTGTTGCAGCCAGACCCAAGTTTTAAAAAAATGAATGGAGCACTCTAAGGTTCTCCACTCT
ATCAAGGAAGCCATACAACTGGGAGCAGCGGCAACAAGTGATATTGAAAACTAAAGTTCAAAGGATGCGGTTCCAGCTGCAGAATAGCACTGCC
GCTGAGTCCTGGGTGGGGAGGGAGCAATGCCTTTGGACTGGTGTTTACAGCCTCGGCTTCCCAAGAATGAGCTGTACACGGGCCAGAGTAGTTG
AAGCTCGGGTTAGACTCTTAACAGCTTCGACAAAGGAAGTGCCTATGCTGCACAGAGCAGACTCACACACACGACACCCACACACAAGCACCTC
TCTCTCATTAACTTTCTCGACACAACCTAAGTCTGTGGAAATGAACCGCTCATTGGTTAACGTCCAAGCTAGGCCTCTGCCAATACCTGCTCAG
GCTTCCATTTCATTGTTGTTATAGTGTCAAAACATCACAGTCTCCCCTTCCACATGGGAAGTGAGGTATTTGAGGTATGTAAAGGAACCATCTA
ATGTGCTGGTCTTTACAACCCTTTCTGCTCCCTGTTCCCTAGAGGGCATCTTTGCACTGTGGACATCCCCCATCAGTAAGACAAGGCCCATAGC
TGGTGATATGCATGTAATAGACATGTGGGAATGTCCTGAGGCTCACTGTCTCCCATTCCTCCCCCAACCCTAGAGAACACTCACCATCGACAGT
CATCTCTGTAGCCCCTCAGGCTCTTACTCGCAGCTAATGCTCATTCTAAGTCAACATTTTACCTCCTTGAGGTATGGATAGAACACTTTGGAAG
GCTCCAGATAATCAAACTTCATCATCGGGTCGTCGGTGGTCATTTTGACAACTACACAGGGATGCAGTAGGGTTTCTGTGATGCTGGGGAGCAG
AAATGAGCCAAGTCACCATGCCTTTAGAGAAAAGAGGCCTAGAAGACTACAGAAATGGAGATCCGACAAATCAAAAGCGGCTCGAGCCATTAAA
CATAAGGCAATTCTAACGCCTGTGATTAGAACAAAAAATAGTCGTCTGCGAAACTATATGTTTTTGAGAGAAATACTAATTGCAAAATGTGTGG
GGAAGAAAACTTCACAGAGGCGGTTCTGTCAATGTCATCTTCTCATTGTGCCTTTAATGCCGTTATATGGGCCTATGGGCCTGAGAAGCTAGTG
TTTGGGTGATACAGTATTTGAAATGATTTAAGAAAACATCGCAAAACCCTGCTTTTGATTTCTAAGAAATCCAGCTTGGACGTCTCGGCTTCCA
CAAAACAGGAGTGTATAGCAAGTCCTACTATTTTGGTGGAAGGATGTGTGCCTGTTTCCTCAGGAGAAAGGATGACACACTACACATAAAATGC
CCAAGATGTTGAAATTGTACTCTACCCCCTTCTTGATTTCAAACACGTTGTGCACATGGTTCATCTCATTTCTTCCCGAGACTCACTCTGGTGA
TCTCTGCCTCCCCCTACACACTTACAAGATGTTCACTGGGAGAAGTGCTTAGCTACAAAGCTGCCTAAAGTAAGCTGCGGTACCCCACAGGTAC
AGGGCAGCTGCTCCAGACACCTCCTGTGGGCAGTTCTGAGGCCAAAGGGGAAAAAAATCTAAATGGACCATCTCTGCAGGAAGAAAATAAAATT
CTTTGTATTCTCCTTCCTGAACGTCTTTCTCTTATGTCCAATTTCTGATAGACTCCAATTATATCAACTTGTCGACCTCCTCCTTCCTGCCTAC
ATACTTGCTTATTCATTCACCCAGGCCACGTGAGCTCGCTCCCAGCATCATCTCATCCAGTTGTGTGGGTTTTAAATCATCTCTAGACTCAACG
CTCCCACTTTACCTCTCTAGAATTAACCCTGTCCCCTGGTCCTTTTGATCCCTTCCTAGGGCCATCCATGCGATGTTGAGTCTGAGCACACCTC
CTCTCACTCCCAAACCCGAGCCTCTCCCTGGGGTTTCCCGTTCACCAAAGCCACCCCCGGCCATCTGGTCACTTACTTGGAACTTCTCAGAGCC
ATCATTAACTCTATTCCTTTACTTCCTCCACCTACTCATTAGCACATCTTACCAGTCGCACTTTCTAAGCACTTGTGGAACCCGGCCACTTTGC
CATCTTTTTTGCTGTTTCCTCCCTCTTCTGATCCATCAGCTAGACCGTTTGCTAATACTACCCAGGAGCTTCCACCATGCCTCCCTGCCAGCCC
ATTCCACTTAAACATCAAAGTGATCATTTTGAAGCATAAATGAAATCCTATACAACTCACCCACTATTGTTATACATAGACTTCAGACTACTCG
CAAACCTCCACCTACCCTGCCAGCGCTTCACCTTTGTAACCCTCTCTGTCCATGACCTCCAGCCACTCTGGAAGGTTAGCATAGTGTCAAGCCT
GCTCATTCTATAAAGTTGTTGAACAAGCCACTTATTTTGCTCTCTGAAATCTACATGGTCTGCTCACTCGAGTCACACAGAATCTGCTCAAGGG
ACTTACTGTGACCAAGATGCCAAATGCAAGACCCCATCCCGAGTTCATCCACTCTCTTCTCATGCCTACACTTTTCTCTCTATCACATTCTGCT
GGGAAACTGTCTGTATATTGATGTTTCTCCTCAGGGTAAGTGCTCCCTGAGACATGTTCCCTCGCTTTGTTCCTGCTGCATGCCCAGGTACCTA
GACGAAAGTATCACATAAGACATGGAATGTTGGAGACAGCCAGTATTCACTGAATGCCCATCTTTGTTGTAGGTGTTCTGATACAGTAAAAGAA
ACCACAAGCAAAATGGGATTCTGCCCTAGGGAACTCACCCCTTCGGTGCCACAGACAGACAAGTACGCATCTCTGTCGTTCTGTGGCTGGGTAG
TGTAGCTCCAGCTTGAAACCCCCAACTGTGCCCGCAGGAATCGGAAAGGTAGCCAGAAAGAGCTGACATTTGAGATTATTCCTGACAGATGAGC
AAGTGTTTTCAAGGTAGAGGAGAGTGGGAAGGACACTTCAGGTGTGGAAGTGGCAGGTGCAGCACCGTGGCACTCTGATAGATAACATTCAATG
GACCGCGAATGTGACCAGCTTATGAGAGAGCTCAAAGATGTGAGTGCTTGAGTCAGCTACCAGAAAGCAACAACTTACTTTATTCTTGGCGCTG
AACGTGCTCCTTCGAATTTTCCCTTTTACTAAGCAGTGCCCGTTCAGATGATTACTCATGCCGCAGTTCTCAGAGTCATCCTTGTATTTTCATT
CTCTTACACTCCACGTCTAGTCTGCCAGCTACACACCATCAGCCCTGCCTTCCAAATAGGTGCCACGCCCAGCCTCTGCTCGCCAAGCTTATAT
GAATGTCTGACCCCAAGCTGCACCGTAGCTAACACTTCCAGGCAAGCCTCCCTGCTTCTCTTATGACCTTCATGGCCCCATTCCAGTCAAAAGC
CAGCACTGTCATTGTCAACTGCTCTTAACTTAAAACTCGAGGACCAACGGGCATGGATCAACTACGTGGGTGCCTAGAAAAGAGTCTCTGTGTG
TGTTGGGGGTGCACTTGAGAGAGAGACAAAAAGACAGACTCTGCATATCAAAGTCTGTGTCTTGTATATCTATATGTCTTCTTTCCTTCCCCTC
CCCTCCTTCCATTTCTTCTCCTTCTCCTCCCCCTCCTCTTCAACCCTCTCCCACACCACCATTCTCCTCCTCACCTTCCTCCTCTTCACCTTCT
CACCCTTACCATANNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNTTTCCTTCCTTCCCCTCCT
CCCACTTTTCCCTACTCTCCCCACTGGCCACCATCAGATAGGAATCCTTGTTTCACTTCCAACACGTGTCCACTGGCCGTGATGTCACACCTCA
CACAGAGACAATCGAGCTAAGTGATTGTAGACTGAATCTCTTGAAAATGTCAGCCAAAATATATCTTCCCTCCTTTCGCTTGTTTTATCTTAAG
TATTTTCTTCACAGTGACAGAGATACAACTAATATTATAAATGCAAAGAGCATGTCGTTTTTAAAAAAAAAATCAAAATAAGCAGAAAACTTTA
GCTTACTTGATCAATAAAAATCAGGGAAGTGCATTTTGATATATAATGTGGTTGCTTAAAACGAATCATGAAGTCCATTTCACTCAGATTTCTA
TAAATAAAATTTTAAAACATAATGAAATACATTCTTTTATGAGAATATGTAATTCAGCCCTCTGAAGTTTAAAGAGAAAGTCTATAAGATTGAC
CACTGTGGGAAAACATGGTTTGTTCTTTTTCTCACCTCCGAGACAAAAAGCCTCATAGAGGCAGCTTACAGGAATAAGAGTTGTGTCAGCTTGT
CGCTTCTGAGGACACTGGTCCGTCACAGTGGCAGCCTTGGAGGATGCGAAGTTGCCACTGCCAGTGTGGGACACGCACGCTTCTGTCTGTACCA
GGAGTTGAAGCACAGCTTATCCACATCTGGAAGATGAGAAACCAGAGTGTTAGAAGGTTAGAACCTTCGAAGCTGACCCTAAGCATCTCGTGTC
TACCAGCTAGTTCACAGTCCCTCCCAAATGCTGCACAGCCTCCAAXACAAAGCCTTCAATGAGGCACCGCGAGTTCACATGCAAACCCACCAAC
CTCCCAAAACACAGCACCACCTGTGCGCCATCCGTTCACATGGAAGACTTAAAATCACCAATGGAACTTCAAGCTTGTACCAGAGTTATAAGAA
CAGTGTCATGGGCAGATGGATTTTATTTCAGGAGTAAAGGATGTTACAATAGAAAAGTTTTAATATAATCTATCACACTAAAATCTACCTTATA
ATTTACTAAGTTTATCTTTTATACCAAACACTATACTGCTTTTATCAATTCTTTAAACGTACAGATATAATCCAGTGTCCACTTTTCATCAAAA
TGTCTTTAAAATTAAAAGTGATCCTTCCTTAACACAACAATGTGGCATAGGGGTAGGATCTCACTATGTACCTCAGGCTGGCCTTGAACTTGGA
ATGCTCCCCCTTTGACTACTGAGGCTGGGCTTAAAGACTTGTGCCATCACACCCCGCTGGTGTAAGTTCACCATTATTCTGAATAATTTTGTCC
ACACAGGGCAAAAGAAGGAAATTAGAAAGAAGGAAAAAGAATCCAAAGGCAGGGGTTAAGCTGACATGTTTTATTTGTATAGGGTATGAGTTGC
TTCAATTTCATGCTCATTTCTATGAAGCAACTAAAATGTAGTATAACAAAACTCAAGCAATGAGTTTATAGAATGAGTATCCTTTGTGTGTGTG
ATTAATTGTTGATTTGAAGATTTAATGAAATAAAAGCATCCTATTTACAACAGCAGGAAGTTCAAATCTCAGTTATAAATCTCAATATGCTATG
TGTATAAGAATTGAATTTAAGAATCTTTAAAGCTCGGTGTGGTGGCGCATGCCTTTAATCCCAGCAGTTGGGAGGCAGAGGCACGCGGATTTCT
GAGTTTGAGGCCAGCCTGGTCTACATAGTGAGTTCCAGGACAGCGAGGGCTATATAGATAATCCCTGTCTCCAAAAACCAAAAACCAAAAACCA
AAAAAAAAAAAAAAAAAAGAATCTTTAATACATATGTGTATGTATCTATGAATGTATGTGAATAAAAATTTTAAACTCTCTAAGATACAGAAGT
AAAAACAAAAATGTAAATAAATGGAAAGGCAATCCCTATTTTTAAATACGGAATTTCATTAGTATAAGTTTAAAACAATCCTAGTAAAAATACA
AGTAAAAAGACTTTTATAAACAAACAAAATGGTGCCTAATCAATGTGGCAACAAATTTAATCAAGAATACTCCAGGAAAGGTGAAAATTATGAT
TTAGATATTAAAATGTATTATATTTGAATAAGGGCACTAATAGACAAATTACTGGTGTAGAAATAGCCTCAATTTTATTTTAAATTTTAATCTA
CATTGGGAGGTTCTCATGTCAGTAATATAAACCTGAGATGATCAATAATTTGTGCTGGGACAACCAAATAGCTGTCAAGGGGAAATCCGCTTGA
CAGTTTCATTTTATTGCAAAATAAAATCTAACTAAATTCAAGAATTTACGAGGAAATTGTTAATATCTACAGGTGCAGCCGACCTTCCAAAGTA
CAATAGAAAATCCAAAAGCTGTAAAGAAAAGAATCATAAAATGAATTCAGTTCCATAAAAATTACACACTTGAGAGGCAGATAAATACAACTTC
CAAAGGCAAGTGACAAAGTAGACAGAACATTTCTGCAACTAATCTATATAAAATTTTTTAAAAACCTAACTCCCCAACCAATAAAGAACCTTTA
TAAGGAACTAAGAAAAAAAATGGCTGTGCAATAGAAAAAAATGTAAGGTTATGCCGGGCGGTGGAGGCACACTTTTAATCCTAGTACTTGGGAG
GCAGAGGCAGGCAGATCTTCAAGTTCAAAGCCAGTCTAGTCAGCCTAGTCTATGGAGTGAGTTCTAGGGCACCCAGAGCTACACTGAGTCACCC
TGTCAGTTTTAAACAACAGAAGAACACACCCACAGAGTCAACTAATCAGCGCTCCCAGGGCCCACAGAGACTGAACTGACAGTCACGGAGCCTG
CATGGGGACTGCGTCCGAGCTGGGGTTGTTTAGCTTGGGCTTTTGTGGGAGTGAGGGGAGTCTTTGACTCTGTGGCCTGCTCTTGGGACCCTTT
TCCTCCTGGGTTGCCTCAGCCAGCCTTGAAATGAGGGTTTGAGTCTAGTCTTACTGCATGTTGTTATTCCAAGTTTGGTTATCACTAGGAGGCC
TGCTCTTCCTGAAGGAAAAAGGAAGAACAGTGGATCTGGGAAAGAGGACAGGGGGACCTGGAAGGAGTGCAAGGAGGAAGGCTGCGGTTGGGAT
GTGTCTTAAAGAGTAAGTCTTAAAAGCAGAAGGTTATTTTCAGTTTACACGAAATATAAACAACGCCTTAGGAGCATACAAAATATAATCAGCT
CACTTATGAAAAATGAATGCAAATCAAATCTATAACACACAATTTTAACAGGAAGATGTTTTCATAGTTTAACAATATGATCTATTACCAAGAC
TATCGATAAAGGAGTACTGATAAATACCACTACTGCTGCCTCAAGTATAATTCTTATTCTCTCAAAGAACAGTTTGGCAGTATCAGAACTCTAA
AAGCAGTCTCCAAAGTATATCCCAAGATGTCACATACACCTGAGACAGGAAATAGCGACAGACCTTGCACGCCAGTCTCTTTCCCCCCTAAGTC
ACCGTCTCACTGACTAGCTCAGTCTGGCCTAGACTCACCAGCCTCCTCTATCGGGCTCCCAACCACACACCGGCTCACCTGTCTCTGTTTCCAT
CTTAAACATTTATCACACACTGTGGCCTGAACTTGTGTCCTGAGGTGTGATAGTAAAGGTGATACGCATGTCTTCATCCTTCCCAGGTTCATTG
ACACAATATTCCTTTTTTACCATGGATCTTAGCAACCTCAGTATATGAGTTCTTTATCTCACCAAGAATTCCCACTTTTTCACTTAAACACTTG
AAGTTTCTTTATACTATTCTGAAAAGCTATCAGTTTTAAAAAAAAGTCGGCTACATAGTTATATAGTGGAATTACATAAAACAGGAAGGAACCT
TCATTCATTCATTCATTCATTCATTCATTTTTGCTTACTCACTTCACATCCCGCTCACTGCCTGCCTCTCAGTCACCACCTCCCACAATTCTCG
CCCCGCCCCTTCCCCTTCTCATCTGAGCAGGTGGGCCCCCGGGTGTACCCCCACTCTGGTACTTCAAGTCTCTCTGAGGCTTAGGTACATCCTC
TCCCACTGAAACCAGACAAAGCAACTCAGCTACAAGAACATATTCCACATACAGGCAACAGCTTTTGAGATACCCTCCCCTGCTCCCATTGTTC
CAGAACCTCATGAAAACCAACCTGCACATCTGCTATGTATGTGAGGGGAGGCCTACGTCCAGCCCATGTATGTTCTTTGATTCGTGGCTCAGTC
TCTGAGACCCCCAAGGGTCCAGGTTACTTAACTCTGTTCGTCTTCCTGTGGAGTTCCTATCCCTATAGGAGTCCGAAATCCTGGAACGACTCTT
TTACCCATGGAATGATACTCAAATTTTATATATATAATAAATTTTATAAATTTATACATATTTATACATATGTGTGTGTGTACGTGTTTGAATA
TGTGTACATATGTATACAGACACACATACGTGCATATGAGTGTGAAGCCCAGAGGATAACTTCCATGGCTTCCTCAATCATCACCATATTATTT
TTTTTTGAGGCAGCGTTTTTCGCTGACCTACAACTTGCTGACTTTGCTACTCTAGCCAGGCACCAGTCCCCAGGGACTCTTGTGTTTCTCCCTC
CTCAGCCCTGGGATTGCAGCATACATCACTACAGCTGGCTTCTTTACATGGNNNNNNNNNNNNNNNNNNNNNNNNNNNNNNNNNNCAAGGTGGA
AATATATATGAATCAATGAATGTGAATGAGAATTAAAGTGCATATCCAGGTGCACATCTCTATCCATATATATGGAGACCAAACAACAGCATTG
GTTATCCTTTCTCAGCACTATCCCTGGCCACCTTCCTCCTGTACCCACCACGGTCCCTCACTGCCCTAGAACTTGCCATCTAGGTCTGTCTACC
AAGCCCCAAGCTTCTGCCTGTCTGCCTCTCCAGTCCTGGGATTCTAAACACACACCACCATACCTGAACTCAGGTCCTCACACACTTCACCAGA
TGATCTGTCTCTCCAGCTCACAGGTAGCGACATTTTAGAGCTCCTTTTTTTTTTTTTAATCTTACTGTACATTTATAATTGTACATAGTAATGG
GTTTCATTGTGATGTTTCCATGAAAGCCTTTAACACAATTTGATCATGTCGAGTCCTTCATAATCCTTCGTTGGCCTTCCTTCTCCTAATCCCC
TTTTCTCTTCCTGGAAACCCACCTCCTCCTTTCATGTCTTTTCCTCTAAATTCCATAATGAAACAAAACGTGATATTTATCTTTATGAGCCTCG
CTTTATATTACTTAATATGATATTATCTAGTACTGTCTGTTTCCCAAAGCACTTCATTGTCGATGGGTGGTTGGGTCAATGGCTGGGTAGATGG
ATGGATGGATAGATGGATCGATGGATGGATGGATCGATGGATGGATGGATGTCACAATTCTGTATACCTTTGGAGACTACATAGTCTGATTCTC
AGTTGAGGGCAAATGTGAATAGTGCCACAATAAGAGTTGCTATGCAAAGTATTCTGACTTCGACTTCTTTCACTCTATATCCAGCAATGCCATA
GCTAGATTCGTAATCTTGGTGTGTTTTAAATGAATGTCTATTCTGATTTCCATCCTATCTATCCTGGCTTCTATTCCAACTAGCAGTGGACACG
GGCTTCTTTTCCTGTGCTGTTATTTGTCGTTCTCTGTTATCTTCGTGGCAGCCATTCTCACTCGGGGAGATGAAATCTCAGTGAAGTTTGATTT
ATATATTTTCTGTGGTTAAAGATGCTGGACATATCTATTGCTTATTAGTATTTCTTCTTTAAAAATGTATCTGTTCAGCTGACTTGACCACGTA
TTGGTTGCATCACATGTTCTTTGTATTTTCTGGATACCAATCCGCTGTTAGATGAATACTTGGAGGTTTTCTCCATTTGTTGTAAGCTATGGCT
TCACTCCGCTAATGGTTTCCTTTGTTGTGCAGAAGCTTTTCAATATCATACAATCCCAGTTGCCAGTTCTTGCCCTTATTTCCTGAGCTACAGG
CGGCCTATTTTAAAAAACAATAATCACTACCTGTGCCTGTGAGCTCGAGTGTATTTCCCTGTTTTCCTATAAAAGTTGTAAAATCTCAGCTCTC
TAGCTACGACTGTTTTGAGCTGATTTCCGTTGCTTCAGTCTTCTACCTGCTCACGTCCACACGTTGAGCAAGATGCTCATTCTTCACGATGCTG
TGTTGTCACCTTGTTGGGAACGAGATGGCTGTGTCCACGTGGGTTTGTTTCTCTGTTCTTTCTCATGGTTTCCATTCTGTTCCTGCAGTAAAAT
ATCCTGAACAAAAGCAATTCAGGGGAGGGAAGCTGTCTGTCATTGAGGTTTTGAAGGGAAGGTGCACTCAAGGGGCTTTCTTACCTGGGCAGTA
ATTCAGCTGGCGGAAAACATGAGCCTAATGTGTTAATACCATGGCTGAATTCAAAGGGGGGAAAATGAGTTTCTCCTCTCCTTGCGGAGCCTCC
ATCATGATTTATGGTTCTGTAAAGTAGGGGGTGCTTCACAGAAACTGTTTCTCTTCATTGTACAGAAGCAGTGTTGTGAAGATGGGCTTTTAAA
AGCCTCCTTGTTTCTCCGGCTAATGCTGAGGAACTTACATGGCCCCATCTCTCCCTGGGCAGCAGTGTGCATTTGTTTTTGATTCACAGGTCAG
CAGGTTTAAAAACATGTGCAGGGTGTGGTGTGTGGCTTCCCATTGGTTATGCATCCATCTCACCCATGAGGTATAATTAATCTCATAGTTCTCA
CCACCATCGGCGTTGTTTTTCTAATTTAATGAAATAATTATCATGTTGTCCCCAGCTTACTCTGTGTGTGGCATGTTGTGGGTGCTCAAGGAGT
ATATCTCAACGGACAGAATTGAAAAAGGAGTTAGCTACTGTAGCCATGTTGAAATTCAACCCTTACAGTCACATTAATCTCTGTTAGGCTAAGA
GCAGTGGCTGACATTGGTGGTGGAATAACTGGGATGGGACACAGAGAATCCTTGAGGAGTGGTGGTCATATTCTGTGATTTGGTAAGGTGTGGA
TTTCACACATATATTCATTTATCAAAACTCAATGGCTTTTTCCACTCAAGATTCATAAAATGTGCTATATGTGTTGTTGTCTCAAGAAAAAAAA
TCCATCAATCAGTCTTAATTTCTAGTACATATTATGCCTCCTGAAATATTTATGGAAGAGTGTGCTCATTTCTGCAATCAGCTTTGCTCTGCAC
TCAAAAAATAATGGATGAGGACATGGGATCCTAGAGATGTGTAAAGAAATGTGTGTTAACTGCAGATTCTAAACAGAGAGTATGTATGGATTGG
ATGTTCCTTATCCAAAATTCTACAGACCAGAGTATTTCAGACTTTATTTTTAATTTTGGAGTATTTGCATATGCTTAGTGAGCTGTTAGATATG
AGATTCCACTGTAACAATTCATTTGTTTCCTGCATGGTTCAGGATATTTGATCATACTGTGAACCCCAAGATTGTGTTGTTTAATGAAAAAAAT
CTGTTTCTAGTTGTGGTATGTCTCAGCACTTAGCACACACCTTTAATTCAAAACAATAAGGTACAGTTAGTTTGTAGAAGGAACTGCCCATGTT
TGAAAGTGATGTCTAATTGAGCGGCAGACAAAGTGACTAATCAGAGAAAGATTTGACAGAATAAGTCAGAGGTAGTATATACCCAAGTCTCAAG
AGAAAAGAGAGGAAGGCCAACCTACTTTAAGTGGCAGCATGCAAAGACTAGGACAGGGGCCAGTTTACCAGCAGAGTTTTACAGACATCGTTGC
AGGGAGAGAACAAGCTAGGCACAGGTGAAGACAGAAGGAGTCAGAAGATGAGAACATATTCCCATAGTTTGAGGCCAGTCAGAACAATTCGGTG
AGAAGTGAAAGAAGCCAGTTTGAATCAGTCAACTTGGAGACGAGTTTTGAGCCAGAACAGCTGAGTTGAACCAGCCAGCCAAAGTTCAGAAAGA
ACTAGGAAGTGTGAGCTTATTCAGCAGTGACTCTCATAGGCTGAAATTATTCTAAGCCTAGATAAGATTTTATGGATGCTAGAACCTTCCAGGA
CTAGGCCTACGTTAGCAGACAGAGCCAGTAAGCCTCGGAGACATTTACTACAAGGAAATAAATGTTACTTTCGCACATGCGTAGTTTACATATA
TAGACAAAAGTCACTTCATCATAATTATTATTTTATACATGAAACGAACTGTCATGGTATCCAGTTTTCTTTTTGTGGTGACATATCAGCACTA
AATATGTTTTAGAGTTTGAGGATTTCATTTTTTCAGATTACGGAACAACTTGTAGTTGTTAGCTTTAATATTATGTGAGTGTTTTTATAAACTT
GAAACCTTTTTTTTAAGGGGAGCAGGGGGAGAGGAGACGGAATAGGGGATTTTTCAGACGGAAAACTAGGAAAGCCAATAACATTTGAAATGTA
AATAAAGAAAATTACTAATAAAAAATATTTGTTTATTTATTTATTTATTTATTATATGTAAATACATTGTAGCTGTCCTCAGACACCCCAGAAG
AGGACATCAGATCTCCTTACAGATGGTTGTGAGCCACCATGTGGTCGCTGGGAATTGAACTCAGCACCTTTGCAAGAGCAGTCAGTGCTCTTAA
CCACTGAGCCATCTCTCCAGCCCCGTACACTTGAAATCTTAAATAATTTTTCTAAAAATCAAAGGGCAAAACCACTCATGACCTTGCAAATATG
ACACCAAAGTCCACTTGACATTTTTTACCCACATTCTTCACGTTTTCACATCTGCTCCTTGAAACAGAAACATCATCATTTGCTACAGACACCC
TGGTGAACCCAGCAGTAGATCTTTATGATCTATAGAGGGCCCCTTTTACTGGGGGTGAGGATTTGAAAGACAATTTTAAAAGGACAGACGAACT
TTATACTTAGCGTGGCGACATACAGGATAGTCTTCTGCATCACCACTAAAAACAGTTCTCCGTCTTATAATCATGTGCATGCCAGAGAGTAACC
ATTTTTATTGTCACCTAAGCAAGACCTGAAGAACTACCATTCTGTGATATTGTCAGAAAATGGCTACCTCTACCTACACCCGGTTGTGTACATA
ACTCCCTTATAGAAATGACGTGGAAGTTACCAAGCTCAAGTGCAGGGCAGAACTGTCTCCACGTGCTTATTCGTACCTTTCCTCTTGGTGTATT
TTTCCCTCGTGCAATATAGTAGAAAGCTAAACCCAGACACATGTGCACCCCTCTTGTAGTTACCAAATCCCTCTGTGATCTTGTGGGCATTGCT
TACTACCTTTGGGTATAACTTTCCACAATCTTTAAGCAAGTTGATTGAGTCAGATCATCTGCAGTCCCTTCTACCATTGGGCTGCACCTGCTGT
AGTGAAATCTTGAACCCATACCCTTGGCATAAGATGAGGAGCCCCTGGTCCCATCCAGATGAGATACAGATCTACAAGAGACTCATTTACATAC
TGCTTAAATACTCACTTTTAGTCAAAGTCAGTCTTCCTTCTGCTTTTCCTTACTATCTGAACAGCCAGAAAGCAAGATGTATTCTAAACCTCTA
CTAAGAGTATCAAGAAAAAAGAACAAAGAAGAAAGGGAGTTGAAGAAGTGAAACTGTACATTTGGTTCTCTTACACAAATCCATGTGACAATTC
TCTTACGCTGCTGTGAATTTACACGGCATTACGCTGCTCTGTCAATTTACAGCCAGAGTCTTTGCCTTCTCAGTGTGGAATGGAACTCAAAGGC
ATGAGTGGTTTGGTCTATCACAGTGTTCAAACCACTCAGAGCCATTAGCTGGGTAAACTTTCACCTCTTCGTGTACCATTTGCTCAACTGTGCT
CCAAAAATCAGAGTTTCAACTTCTACTGAATATGTCCTTGGCTTACTGTAATTACCCAAGGCTGGTTACATGACACACATATATTTGGATTCAT
AGTCACACAGTGTACATTATAGTCCAGATGTGGTTGTATCCACCAGTCTTTATTGTAGTGTACATGCATGTTTGCATATTCGGGTATGGACTCC
TATGTATTGTGTCCATTTGATTAGCTGTAAACTGGATGTCAGTAGTCGACCTCCTTAACTAAAGAGAGAAGCTAATTACCAAGGACTTGACTTT
ACTAATGTACTCACGCTGAAGATGTGTTAGGTGTTAGGTGAGATCATGGACAAGCCATCTGATTTGGTCCATTATTATATTTTCAATTATCTTT
CATATTCAAACAGAGACATTAATATAGCTGAGGATCACATAGCAAATTACCACAGCTCTCTAACCCTTTAAATTGCTAATACTGTTGTAATCTG
TAATTATAGAAATCAGTGTCAGAATTGTGGCAGTGCATAATTATCTTCCTGATACTTTATTGCTGAATGAATTATTAATCAGAATCAGTCTTGT
ATAGTATTTGCAGCAACGCACAAGAATAATGTATAATTAAATTTCATTATTCTGATTTTTGTGTTAATGTTTTATCATGTTGTAATTGATAATT
AATTGGCCAAAACTCATACAGTACTTGGAAGTTTCAGGTGGCTGGGAAATGTTCAGTGTGCTGAGTTCATTAACTAAGCAGTAATCATTCTGCA
AAGACCAAGGGATCTTGTCCAAGCTATGTGAGAGAGTGCTTAAAAGTGGTGTCTTAAGCTTGAGCTTTTCACATTTCCTTTAAAAAAAAAAAAA
GCAATAACTGTGAGCATGTACAAATGATGAAGACATCTTGATAGCTTTCAGATTGGGTCAATTTTAAGTAATCAAACATAGGTTGAGAATGTCA
GACTAATACAGTCGGTAATTTGGTATGCCTCTCTATATCTCAATTTCCATCCTATTCTAATTAAACCATCCTTCTCCACATTACTATAGTGTGA
GGAAAAGTGCCACGGCCATAGACCTAAGAGCTCTTTCCTCCTGGTCTTGGTGAGTATAACACATCACATAGCACATCCTTGTAAGGAAAGTCTG
GTTTCATCTTTCCCTATTGTTCAAAACACTATATGCCCCCCTCCTCACAGACACATAGATTTAGACTCTGAAAGGATTATGAACTTCTCCTTGA
CTGGCATTCTAAGCAGCAATTCCTGGAGTGAGTTTCTTTTAGAATCCTTTGCAGCCTAGGTAACCAGGAAAAGCGCAGAATCTAAGAATGCCTT
TCACCCAGCCTGATTCCTATTGAAAAGATGCATTGTTGCGCATTTTGTGGACTGTGCTCTTTGATTCGATTTTTAAGAGGCCAAGGAATCTTTT
TCGTTTTCAAAGTATCTGCTTTAGAAACATACAATTTTTCTCTTAAGCAGGTGAGCCTGTTTTTACATTCTGAGGAATTCTGAAACATTGTATT
AGACAATGTTCACAGTCCATGCTTATTTACGTATTTATTTATTTATTGTCTTTTTCTGAGCAGATTAGAAAACCATGGCGGGGAGCAACAAAGG
GGTTAGGGGACTGTTTCTGAGATTCCTGAACAAATTTGCCTCGACATTCCCAACAACCTCTCAACTGGCTTATTTTTGTGAGTTGCACTTGCTT
CTGATGCCTTCCACTCCCATTTACTCAAGAGGAATCTGTCATCGGACCACAGGAAAAACGCACTTGCTAGATGCTCTTCATTGATCTTATTCAT
CTTGCAATAAACATCAGCAGCAATCCTCAAATCATCTCCTCTCCACTCCACGTACTCCACAGCCATCTTGATCCCAGGGTACCCACAATTCCAA
GGCATCACCCCACTAAGCTTCCTCACACTAACCCCCTTCTGAACTCTCAGCTGACGACCTCTGCCGCTGTCACCTTCTAAAAGCCAGTCCTGTT
GAGTTTGAGGTGTAAGTCTGTGTTTATAAACAGGCGGGGTCAGAGTCATCTTTCATATGCAGAAATAAGGAAGGTATTGACAAAATAGAACAGC
TAAACCATATTCTACAAGGATCCACCTTGTGTGCAAACAGCGGATAGAAAACGCTCTTTGGTTCATGAGTAGTTGAGTCCTTCCCTTTATGTGA
CTTTGTATGACTTTAAAATAGGAAGAAATCTGGAGTATGCAAGCCAGACACTGGAGACACTGGGGACAACTCACTGAGCAGGCAGTATATGAAC
AAAGACCTGAAGTTGATTAGAGACTAAGCAATCACTTTCATCCCGAGGCAGCCTCTCAGGAGACAACACGTTCTCTTCATCTCACATATGAGGA
GACTCGGAGAATGTACACAACCTGCTCAGACCACCCAGCCCTAGGAATTCCCAGTAGGTTTGGAGGGGAGGCGAGGAGGGCAGCCACTTCAGAT
CAACTGTTAATGGGGGTAAAATGCAGATTTCTAACCACAGTAGTTACACTAAATGCACATATGCAACTTGAGAACTATTATCATTCAAAGAGAT
AGGAAATTAACTACACAGTCTGAGGTGGGCAGGAGGCACTGCCTCAATTAAGAACTACAGGTTACAAAAATATAAGCCACTGTCAATCTGGGAA
TGAAGCGTCGATTTTAAAAGTTGGGATGATACAAAAACTAACTTTATTCATTACTTACTAGGTATTGAATGAGTCAACTCATGAACTAATGACC
AAATGTGCAATGCCTTAATCCTCACAACCCTAGCTCTTTATTACACCAGTTTTAAAGAGTTGGGCGGAAGGAGGCAGAGAAGGCTAAAAGACAT
GTCTGAGGTTCAGCAATAAGTGAGAAACAGGACTCAAATCCAAGCAGTCTGCCTCCACCCTGCGCCCATCACCATTGCGTTCCTCTACCCCATG
TGCAAGCTGTACTTAGTGAGCCCTGCATTCCTGCTCAGGTCCATGTCCAGGCGCCGTGCATGCTTGTGTGTTAAGTCCTTCCCTCTGAATGGCA
TCTCAATGCAGATGAGACACCCTTACCCAGAGTTTCCATGGTTTTCTGAGATCACAGCCTGATCAGCATTCAGATTCAGGCAGGCACGCCCCAG
AGAAAGCACACACATCTCTACTCATGAAAGAAGAGTCAGATGTATATAAATCCAGTGGAGGGAAAGCAGTGCAAGTCTGTCCCAGAGAGGAATA
TGCAAGGCACGTCCGTGTAACAAATAGGAAGCGGTGAATGTCTTAGGAAACCTTATTAGATAACCATCTCCGAGAGTTCCCCAACTACCAGGCA
CTCCCTTTAACATCAGAGAGAAGTAGATCAGCAGTTACATGTCCATAAGAGAAGACTTTCTTGGTCTAATTGTGTAGATTTAGCCAATCCGTGA
TGAGTGTGGTTCCTGAGTGCTTCATTCAGAGTGCTCACTCTTAGATCCATTGCCTTCTGTGACCTTCATGGTCCCAGGGGGACGCTGTGGAAGG
CAGGCTATAGTGATGTCATGGCCACAGCTGCCAAGAAGCATGGGAGCCCACACTTTGTGCCATCCTTGCAGCCTTAAGCAAATTGGCTTTTACC
CACGTTAAAGCGAAAAATCCAAGCAAATGAAACAAATGCCTTTTGTTCTTCATATGATCTCCATCGACCTCCATATGACAAGTCCTCCGTGTCT
ACAGAATTACAAACGTATGCACTGTGCAAATAATTGTTTCTCATGATGATAGTTTGGGACGGAAGATGTAGCTCAGTGATGGGGCAGTTCCCTA
GCATCGGATCCCAGTACCACAAAGAAAAAATTAGTGTTTATTTGAGCACTTACTACAAAAGTAAGTAGCATCCTAAATACTCTATTCTTAGAGA
CAACAAGTGTTACAGAAGCCAATTAATGTGCCAGCATCTGTCATCTAAGAATTAGACAAGCAGGAATCAAATGGTAACCGTCTGTGTACGGAAG
GAAGGAGAACCGGCAAGTGGGTGCTCTGTGGACTCAGGAAACTAAGCCGCCTCTTACTGCCAGCCCACCCAAACAGTTTTGAGTTACTCATTCC
TGACCGTTCAGGAAAAAGAGTATTTTCTACCCCTGGAACACAGCAAATTTTGACTTTTTGTAAAGGGTAAGAGAATAGGGCTAGGAAGATGGCT
CACTGGCTAAGAGCAGTCACTGCTCTCGCAGACGATCTTGGGTTTGATTCCCAGGACCCACCTCAGGTGCTCATAACTGGTTGTAACTGCAGTT
CCAGGGCCATCAAACCTTACCCCCGTGAGCAAGGTGCACATAAATTCATGTAAGCAAACACACGTAAACATAAATATTTCTAAAGGCTCCTTTC
TTTCATACAGTTTCCCTTACCCAGAAGACACTCTGTAAGGCAGAAACGTGTGAGTTCTGCGGCACCGCACCTTTATTCCAATAGCATGCTCTGG
GTGGTGAAGCATCTGTGTCACATTTGGTAATCCCTACTCGAAGATGCTGCCAGGGAACCCTCCACGCCCACGGTTTCCCCACCCCCGCTTTGCT
GACCTCTGCTTTTCCCGTGCTCTTTCCAGAGACTTTCGCCTCTGCTAATCTCCTCCCTCCTCGGTTGGCCTCCAGTACGGCGGCAGATGGTCCA
AGTCCTTGTATTACTGAATGTGAGATTAACAAACTGCTGGCCCATCCTCACCTTGCGAGTTTAATCACTTTTAAGTAGAACAGAGAAAGGAAGT
AAGGGAGGCAGACATCTGTAGAAATCCACAAGCCTTTCTCAGAATACCTGAGCTTTGATTCGCTGCTGCCTGGTTTGGTATGCTGTGCTTTTAT
TTCCTTAGTTACCAGGGGAGTCAGGTGCAGTCTGAGCCTCTTCCATCCTGCTTGCATTCCTGCAGAGAAGATGTTTCAGAACAACACGGATCCA
CAGGACCCAATGGGTTGGGGGTGGGGGGTGGGGGTGGGGACAAGTTCTCATTTCTTCAGAACCTCCTCGTGGGCCAAAGGGGTGGTTCAGCAGG
AAATGAACTTGCCTTGCAAACCTGAGGACCCATTAAACTCGTGATCCCACAAACGAAAGAGGGAACTGATTCTCGAAGGTTGTCCATTGGCTTC
CACATGCTCACACGCATGCTCACATGAAGGTTGTCCATTCACTCCCACATCCTTACACGCATGCTCACACGCCATACACACTAATACTATTTAT
CAGTTACATCAGAACAAACATGTGAAGAACCTGTATTTCTGCAGCCTTTGCAACTGTGACTCTGCTTTCTTTCCCCCTGTAAGCTATGTGCCCT
CTTGGGAGCCTGGGAGAGCATCAGCAGCAATCATAAGAGTGAAGAGTCACCTAGCCGCAGATGCTCTTTGTGTAAGGCAGACCTTTCCCCTGAC
TGTGTCTCTCCAGGTGGACCTGCACACAAAGCAGGGCTTAGCTTCAGTGATGCAGTCTCCCGCATCCTAATCATAGCCAGTCGGATCTCCCTTT
GGGAAGTTTCACGTAGTAGCACATGTAGGCGATACTAACCTGTGCTAGCCACAGTGACATTGCTAATACTGCCTCTGAAATCCGCATGCAAACT
TCAGTCTAGTATTGTCACTTGCCTGATGCCTAAACTTTTGTTAATTACCATAGCAAATCACTTGAAACAGACTCAATAAAAACAAAATTATTTT
TAATTATAGCATATGAGTGTTCTGCTCTCGACGCTGTCCTGGTCCCTCCCCTTTATTTTCTCTCCTCTTCTCCAATATCTGGCTCCCCGACACA
TCTAAAGCCCTCTTAGAACTGAGTTTAGTTTCTCTCTTCTATCTTAGGGCTGACAATCATGATGAACCTAAAATTACCCAGAATTAGAATTACA
GTGCCCCTTTTCCACATTTGACTCTCTGTTGTCTTTTCTTAATCTTTGATTCTGTTGTGAGTTTTTTTTGACTCGGTTCTTTTTTTGAGAGAGA
ATAATTTCTGCCACATTCTGCCAACTCCCCTCCCACCCGATGGGGTCGGTCTGCGGAGGAGAAGTGAGCCCCAGTTGAGCCCACCGCTCAGTAC
AGGTCTGCAGCGCCTTCTATTAAAATGTAAAGTGATCCGTTGAGTCAGCCTCCCTCTGGTTTGACTCAGACGCTTTATGGTGCACTTTGCATGT
CATTTCTCTGTAATCATTTCCCCATGGAGAAGGGTGTCTGATAGAATCCAGGCAAGACTCAACATTGCTCTGAGTCAAATGGATCTTTTATTCT
TTTAGTATTAACCCTTATCTCTCAGTTTCCCATCCTCGAATTTTCCCTCAAGGAACCTTAATGAAGTAAACAGGCCTCAGCACCCTCATTTAGC
TTCCTAACCACTGTTCTCAGATTCTTCTCACGTTAACAGTGGGAAGCTCCGGGGGAATAGTCTGTGGTAGAAGGGGACATTGAGAGAGAATATT
CACACAGATGCCCTCATAAACAAAAATGTAGAAAAGAGATGACATTTCTCTGTCCTCTAAGACGCAGCTCCTAATGGGAAGCAGAAAATCAGCT
ATGAGAAATGAATATAAATGGAGATGGAAGAAGTGACACCAGAGACAAACCGAATGTGACAATTTGGAAAAGAAAGGTGACCAATAGCAGAAAA
TGGCCTGACCCAGAAGAATAGACACTGTCACCTGTGCCCTCAGATTGGTGGGGTGGGGGGGAGCGACGCAGCTTTGCCACAAAAGAACAACAGA
GGAGCAGGGACGGAAAGAGAGATGGAGAGAGAGAGAGAGAGAGAGAGAGAGAGAGAAGGCACGCCATCAGAACAGAAGAACAAGGTAGACAGCC
GCTTCCCCTGTTGTGTCCCTTCCCAGTTCCACAGTCCTCTCCCTCCATTGTCCTCTGTTGCCCCAGCCATCACTGACTGCGACCACTCATGTTG
GACTGCCTGGCATAAAGACTCTTTCACATGACAATTTCTTTCTCTCTCTTTCTTCCTTTCTTTGTTTCTTTCTTTCTTTTTTTGTTTTTGTTTT
TGTTTTTTCGAGACAAGGTTTCTCTGTGTAGCCCTGGCTGTCCTGGAACTCACTCTGTAGACCAGGCTGGCCTTGAACTCAGAAATCCGCCTGC
CTCTGCCTCCCGAGTGCTGGGATTAAAAGCGTGCGCCACCATTCCCGGCCACAGGCCAATTTCAATGCTGCATTCATTTGAGAGTCGTAGTTTA
CGCCCCATCTTACTGTTCAGTTCAAGCTGTGGCCTTCTCCCTGCCTCCTAAGCAGCATCACTTCACCTGGGGTAACTACAGAAGGAAGAGACAA
GCAGCACCTCTTACGTGAAGCACTGAGTCCAGCCTGCCTCCCAGAGCCGATTGAGGTGGGAAGACGTGTTCCTCCGTCCTACTGAGCTTTGTGC
TTTGCTAGATGTGAAATTGGCTTCTGAGTCACCTTTTCTTTCGACTGACTTGTTTATAGACGGGGAGGGTCACAGCCTTACTGAGAATCTTTCT
CTCTTCAGCTCGCTCCAGATACATAGTAGCATTTTCCATCCAACCAGTTACATTGTTGTTGATACATCAACCTTTCTTTTAACAACTGTGGTTA
GAAATGCTTGCTTCGACCCGGGGCGTGGTGCTTTTAATCCCAGCACTCAGCAGCCAGAGGCAGGTGGATTTCTGAGTTTGAGGCCAGCCTGGTT
TACAAAGTGACTTCCACAACAGCCAGGCCTATACAGAGAAACCCTGTCTAGAAAAAAACCAAAAAAAAACGAGAAAAAAAAAAATAAAAAAGCT
TGTTAAAAGACTTTTCCAAAAGTAATACTTTCATAGAAACACAGATATTCTAACTAGGAATACTTTGAGTTCACCTGTTTCTTCTTGCTACAAA
TAAGAAAATGGAGATGCAGAAACTGATTCGCTTTTGTGAGAGAGGAAAGCATGGGATCTCTGAAAATAACTTACAGTGGGACAGATGCTGCGCT
CCTGGTGATCCTAGCGCTTCAATATGTCAATCAGTTGGGAATACAGATAACTTTACTCAGTAATACTGTCTATCATAGGTGTTTATCATTTGTT
CATTTTTAGTACAATAATAGAATTGTGGTATCATGTTTTATGTCTATTATAATTATATATATATTATATTGCAATTTTTACATTGCTTCTGTGG
ACTGACATTCAGTGTTAGGTTTGTGACGTCAATCTGCATGCTACATATGGCTCTAATTCCCTCTTATTCCATGTATAAATAAATAAATACTGTA
TACCTCAGTGCTACTTACAGGCATTGCATTGTTTCTACTAGAGGCTTATTATAAATAATGCTGCCGTGAATATCCTTATTCAGTGTTCCACTTT
GGAACCTGGGAGAATCTCTACTGTAACCTAAGAATGGAATTGCACGGGATAACCCATGCATGCCTTCAACCTCGCTAGATAATGCAAGTTGTTT
CTAGAGATACTAAGACAAGTCTCATCAACAAGCAAGAACTCCTGTTGCTTCAGCAACAATATTAAATTTAATATTGTCAGATTTTCTACTTCTT
TCCAAGCTGGTGGTTATGGACTGTCTTTAATACTTTATTATCTTTAGCCAGATTCATTACAGTTTGCCCTAGCATCATATTATATTTTCCAGAT
CAGTGTCTTTGTCAGCTTCTGCTTCTTCACTCAGATTATTTTAAATATGAAAAGTTAGAATGAGTCGCCATTTTCTCATTGGATACTTTGGAGT
TTAGATCTTTTTTTTTTTTTTAATTTTCATCTGTGCAAATGAATAAATGAAACAACCACCACAGATGCAGAGCTTCTCGTGACTGCTCTGTGTT
CAAATCTGGGTGTACTTCTCAACTGTGAAAGTAAAAAAAACCATTGTCGCAAAGGTCCTAGTCAATCCAAGTTCCTCCTGAAAGGTCAAGGAAA
ATGGACTTTTATACTGAACTAGGGAACCTTTGAGAACTCTGAACCACTTGATGATACAATTGCAATAGCTCCAGTCTTACCACTGCCCTTAAAT
TTCAGTTCCCTTGGCGCTAAGCAGGAGGCAGACAGATGCGGGCTTTGAGGAAGTAAATCAGAGAAACCTTTTAGCTGATCCACGCCAGGCCCTC
CCTCTAGCTCTCTAAGGCACTAGGGACATTAAGCACTCAATGTTTGCATTAGGAAAGACTGAAGCTTTCTACTTTCAGTGAGAAGTTTGATGTG
CTTAGAAACAGTTTCCACACCACACTAACACCGACTCTGACTCCGTTTGAATACCTCTCTGCACTCACGAGTCTCTATAGACATGCTAACTTCC
TCCTCTAAAAACTAACTCTTACGAATTTATAGTAAATAACTTTTTAAATGCTTGATAAGAGCAGAAATTCACTCGGCAATTACTAAATAATTTA
GCATCTGATTCACAGAAGTGTTTACTTAATGCACTGTTTCTGGGAGCGGTCTGCGGCGCACCGAGTGAAGTGGTTTATAATTTACTCCTTTTCT
TCACCCGCAAGGCATATTAATTTTTTTTCATCCCAACCATTCTAGACATGACAGGTTGTCTGTAAAATTTAACATTCTACTTTTTTTGTGAATT
GCCGCTTTTCTGATTCCCCACTGTGGATTTTTTAACTTTCTGAATTATACTTTTAACAGAGGAACTGTTAGACACTTCACTATCTGAATATGAT
TCAGTAATGAATGAAGAGCGGGTAAGCTCACAACTGAGTCTGCATTCATTAATACGGCATGCTTGACTGACAAGAGTGTGGCCTCCGTAGCTGC
TTTTGAGGGGATGAGCATGCTATCATAATATAGTAGTAATTAGCTTCTCCTGCCGAACCATATGAGCAAATGATTGGGGTGTGTGTGTTGTGCA
CATGTGCAGCTGGGGTTTTTGGCATGCAAGAAGAATGAGATCAGCCAAAACCCAAACCAGAGCCTTTGGAAAGCTCCCTCACAATCTGACTTCA
GCTAATGTCTACATACTTTAACACATTGTCTACTGCTTAGGGTAAAGCCTGGCTGTCAGACTCCTGCGTGCTATTCTGGGATCCCTGCTCCGCG
CGCGCACTGTGAAGTGTTTCATTCTTAGATCCTGTGATCCCTGCTGCTTTGTTGCATTACGTGCCAAGCGTTGCTTAATTAACATTAGGAACGA
ACGAAGGAGAAATAGAAAGCAGGGTGCTCGCTCCTGCAAGGGAGGAAGAGCCGAAGGCAGATGAGAGTTCACAGAACACACGTGATGAGGCCGG
ATGAAACGGCTGCACGAATAGCGTGCCATTGTGCTTGAAGAGACCCGGGGAGGGGTGGATACAGTCAGACATGTAAAGAACAAGAGAAAGCCAA
TTGGACAACTACAGCAGAAAGAACTATCCAAGAATCAGGTAACAGCATCCAGAAAAGTCACTAAATAAAGGTCTAGCAATCGTTTCCTGATAAT
GGATTTCAGGGTTTGCCTTGTGTGCGGGTTAGCTTTTATGTCAGCCTGACACAGGCTACACTCCTTTTGGAAGAAAATACCTCAATTGAGAAAA
TGTCCCCATCAGACTGCCCTGTGAGCGAGCCTGTGAGGCATTTTCTTCATTGATGAGATGTGGATAGCCCCATCCTTCATGGGTAGAGAGACCC
CTGAATGGGTGATCCCAGGTGCTATAATAAAGCAGGCTGAGCAAGCCATAAGGACCAAGCCAGTAAGTACCACCCCTCCATGGCCTCTGCATCA
GCTCCTGCTTCTAGGTTCCTGTCCTGACCTTTTCTCAGTGATAAACTGATGTGGAAATACAATTGAGATAACCCCTTTCTTCCTTCAGCTGCTT
TCTTCTCGGTGTTTTATCGCAGCAATGACTAAGACACCTACCATGTGGAAGACCCTGGGTTCATTCCCCAGCACCACCCAAAATAGATTTCAGT
TTCTACTGTTTGCTGAACAGAATTAATATTCTTTAAATGTCTTCAAATCTTAATTGATGGCTTAGCCAGAAGAGCAGATATCCTTGCGAATCTG
ACTTTTATAACATTCTTCGCCAACTGAGACTCCATATATGGCCCTATCCTCTCTTTTCAAAAGCCAGTTCTCTTAGGAAAAGGCTGGGTCCTCG
AGACTCTCCCTTATTCCCCACCTGTCTCGGAGCTCTGCAGCCTTGCCTGAATCCTGGAATATGGTAATCCAGTCTCTGCCTTGTTTTGCCAACT
ATTAACTTCCTCGTGGGACATGATTCTTGTCCTCCTCTCCAAAGTGGTTAGGACCTCAGTCAGGTCTCAGTGGAATCTCAGCTGAATTTCCCTG
GTGACATATCTGCAGTTCATATTTGCTTGCTCAGGCGATGTTCCCAGAACGGACCCTCCAGGTTAAGGTGAAGCCACCAACAGCGTGACTTCTC
CATGTTGCTCCCAGAAGCCCTTCACACTTGGGTCCCCATGTCCTACAAGGAACTTAGTTCAAGAAAGTTACTGCCCTATCAAGGAATATCGCGG
TGCATCCTGACAGGAGTTCCTAATTTGGGCTCTGCAGAGCAGGAGACTGGAATCCTGCTTTCTCCCCCAACCCTAGAGAAACCTTTGTACATTG
TCTACTTCATTTCTTAGAACCACCTCTTCAGAGACAACTTTTTATTTGATGCAACTTCTGCGACAATTAAACTGCACTCATAAATTGTCTTCAA
AGGGCCTAATTAACATAATTTCTGTCAACTAGAAGGTAACAACCCACCCTCTAAATCACTTGGCTCAATTTCCCTCACAGCCTCCTGAGGGTTT
TAGAGCTGTGAGATCCCAGCTGGACTTGTGGTCCTTGCTTCACATTTTGTCATCCTCGATTCAGATACAAAGTGAAGCAGCAGTTTGTCCACAT
CCACAGAGCGGGTCAGTGCCACAGCCGAAGCAGAGGCCACTGCTCTCTCCGCCGCACCTCAGTTTCATTGCTCTCTGGGCTATCAACCCTTCCA
TATTTCACTTCATTTATGGCCTTCTGTTCTTCAAACCAAAACTCTGTCTCAGTACAGTTACAATGATAATAGATTGCCCATAACAATATTTGAA
AAATGGCACACTTTATTAATCCCTTCTTCTGATGACGTTAGTGTGTCAGCCATCTATTTTTTTAGAACATTAGTTGCTCAACCAGGTTTTCAGT
GTTGCCTCGTACATATGAACTGTTACATTTTTTTAACTCAATACATTGTGTAGACTAATAGAAAAATCAAATGTGATAGGACCAGAGGTAATAA
TTTGAGAGTGGAGAAGTCAGGATTGAGACTGTATGGTAGACACAAAAATTCTGGAAACATTCCAAGGAAGTGGAGGCTAATTAGAAGACAATAG
AGAGGCTCAGCAGCTAACAACACTTGCTGCTCTTAAGACAGCACCTGAGTTTGATTCCCAGCACCCATGACAGTGGTGCATAACTGCATCTTAC
TCTAACTCCAGGAGTTCCAATGCCCCTTTCCTGTTCTCTGTGGGTACCTGCATTTGTGTGCAAATGTCTTAGTTAGGGTTTCAATTCATCCAAC
AAAACCACCAAGACCAAAAAGCAAGTTGGGGAGGAAAAGGATCATTTAGTTTACACTTTCACCCTGTAGTCCATCTTCAAAGGAAGTTAGGATA
TGAACTCAAACAGGACAGGTACCTGGAGACAGGACCATGGACGGCTGCTGCTTACTGGCTGGCTGGCTGTGGCTTGCTCGGCCTGCTTTCTTAC
AGAACCCAGAGTCACCAGCACAGGGATGGCACCACCACAATAGGCTGGACCCTCCCCCATCAATTACTAAAGGAAATGCCCTACATCTGGATCT
TATGGAGCCATTTTCTCAATTGACCTTCCCTCCTTTCAGATAACTCTAGCTTGTATCAAGTTGACATAAAAGTAGCCAGGATACCATATGCAGG
AATACACACATATACATAATTTAAAATAAAATAAATCTTTTTCAAAGAAAGGCAGTTTAAAAAAAAAAAAGAACTTGCGTTCTTTAACTCAGTA
ATAAAGAATTTACTAGCAATTTTCTAGAGCTAGACACCAAGAGACCTTGACTTTATTCCATGTCTTAGGACTGTTTTGTATGAATTCTGTCAGT
TATAATGTTATTCTTCGGATTTCACAGATTGTACATAAACAACTTTACAAATACTTCGTTTTTATTTGAATGCCAAAGAACTAATGTGTTTTCC
TCACAAAAACTTTTTGTAATTAGATTCTGAAAAAAACACCACTTATAGCAGAGCATAGTTTACACTGTACTGTTAGCAGTGTGGTACGTTTTGC
TTGTAAAGTACTAGAGAAATCATAACTAATAAATTGAGTGCCGCTGAGATCACTGGAGTGGGCAGGTCAGGGTGCTTCCCTTGCCCACCCTGGA
GAGGACTGGCACCTGTTAGTTGTCCTCTGACCTCCACTCTAAGCAATGGCACATTCTCTCGCACACATGCACACACACAATAAACAAATGTTTA
AAACCTCTCTTAAAAAATAAATTCAGATATTTTGCTTCTAGCCAAACTTGAGCAATGAGTATTTTTTTTTGCATGTAAAATATGTTTTATTGTT
CTTCAAAAGGGTTCAGGGTTTGGTTGTAAATCAGGCTGCACACCTTCCGTCAGTCTAACCTCTCTCACAAGTCAAACTGGCTTCAGTTAGCAAT
ACTTCATTAAATCCAAAAGAAAAAGAGTTTTAATTTTGAGGGGAGGAAAAAAAAAGTCCAGTTCTCAGAACAATTAACATTATTAGGGTTGCTT
TATTCTCCTTCACACACCCTTCTCCCCAGAACCAAGAATGACACTATCTCAGGCAGGCTGATCCTGGTGCACACCTGCATGTGCAGACCCCATG
GCAGCTATTGTGCTCATGAACAAATACACACACACACACCAAAATTCTAGCATGTCAAGTGGAAAAGGTTTAAGTGGTATTGATCAAAACCCTT
GAGGAGTCAACAGTTAACCTAACTTTTAGGTAAGAAGTTTGATCTTGAGTCTTTGTGAAAGAAGCATCTATTGCCATGCTCTGAAGTAAAAATT
TCTGGATTATATCATCATCATCATTTTTCAACTTTAACATTCTAGGAACAGTGTCAAAACCATTATTTTTCATTTTCTGGGATGAGGCAGGAAG
GTATTTGCATTThGTTAAAAATCAAAACAATAGCATCAACAGTAAGCAGGCAACTCAGAAAGCCACCAACATCTGGGTATTATTAATGAGATGT
CATCCATCCTACTGGCTTGACAACACCCAGCAGTGAGTATTTTATTTACACTTCCACCTGAATAACTACTAAGATAAAGTATGAAGTAAGGCTT
TTGAATACCTTCGAGGCCTGAGCACATAGCTCAGTTGGCAAGTAAATGGCATGTGGGTGATAAGACCTGAGCTTGATCTGCGGAACCTGTGTTA
AACGCTCTGTGATGGCTGCAGATGCGGTTCAGCTGCCAAACAGCTCTGCTGACCAGCACGThCAGAACATTAGGCTTGGTTCCTGCGCTACGTA
AAATTAGCTGTGGCATCGACTGCCTGTAATCCAAGCACTTGGCAGGCCAGGGCAGAAGGAGCAAAAGCCTGTTATATATACATAAGAGAATTAA
CCAAAAAAGGCAACCAAGGTGGTCCATGCTTGTCTTCTCAGTGCTGGGTATACAGAAACAAGCAAACCCTGGGGCCCACTGGCCAGACAAATCA
GTGCTCTCTAGGACAGTGGGCAGGGCGGGGAAGAGGAGACAGACAGACACACGCACATGTTTGACTCCTTATGAACAACACTTGAGGTTGTCTT
CTGGCCTCTATAAGCATGAACACACACATTAATGACATTTCACATTACATATTAGGTGGCAACGAATAGGAGTCCCCCAAAGACACTAAAGGCT
CTAGCCCCAGAACACTGCTTTGCGAGTTTCCACACCATGGAGCAAAAGAAAACAAGGCATTTTGACGCTGTCAAGTGGACAGAGCTCAGTATTT
GGATACATGGTTGTGGCCAGACAGAAAATAGGAGGGGGGGGGGGCAGAGAACAGAGAGCCCCAGAGGTTCCACAAAATCTCCCTTCAGTGTTCT
TCAGAGAACTGATGAGCACAAGTGTGCAAAGAGGCATCTCAAACATCTCAAACTCTTCTGTGCAGATAGAACTTAGTGCTTGGAAAGTCTGGGG
TCAGTGCCTGTTCCTCATCGCCCCAGATTTAACCAGACATGAAGTACAGTGCTCACAGATGTCATTGATACATAGTGACGAATAAGGCTCCCGA
GACTACATGTTCTTCTTCATACAATCTCGAATGCACAATCTGAAAGGGTCAAGCTGCTTCCCAGAACCTTATCCCTGTCTTGCAACAAAGCTTA
GAACTGTGTGTCATAACCCAAAATATCCATCATAAAAATTGAGTCCAAAATGTAACTGACAGTGTCTGGCATCTGGCCAAGTATGTCCAATAAT
TAAAAGAAAGGAGCGTTGTAGTGGGGGGATCTGAGCCAGAACCCATACACATACCGAATACCATGCAGTAGTGATTGTGATTTTCATGTCAACT
CGGAAGCTTAAAATTTTTAAGCAGAGACATAGAAGACACTGTAAAACACCCATGTTCGACTTCTTTATCAATCCCTGACGTCACAATGACATAA
AAACTGCATTGCCTTTGAAAAAGATGAATCGCAGAGTAGACATTGCTGAAATGGAGACCGTCCAGGCTGGAAAAAGCAGAGCCATGTTTGAATG
AGCACGGTGGTTTAGAACTCTAGACAArTTCTTATCAGCAAGTCTTGGGCTGTCCTAAGCAAAAGAGTACATGAGACAGCAAAAATATTTGGCA
GAAACAGTTGAGAAAATGATGGCTGGAGTGTCATCAGGTGTGAAAGCTCCCACAGACCCAGACACAGGAAGGAGAGGACATCAGTGCTCACCAT
AATCACACTGCTTGGAAGAGGGAGGGGTACACTTGGAAGTAAAGGAAGGCTTAAGCAAAGGGGAAAGGAGGAACAGAACTAAAGGTGCCAGGCC
ACGCCCTCTCNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNANNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNATGAGGCAACCGAATGTTTTAATTTACAGACAGCATAATCTTCACCGTAG
AAAACAAAAGGCTATAAGAACTTATAAGCAAGTTACGCACGCTTCCAGGACACGGAGTTGTTTCTTGGTGCTAGGTAACTCAGTGATACAACAC
GTGCCTAGAATTCCAGCAAGAGGCCCTGACTTAGATCTCCAGCACTCAAAAAGAAAGTATTTATATATTAACAATGAATAAACAACAGTTCATG
CTGTAAAATATTTGAACACTTAGGTATAAACCTCACAAAATAAAATATATTAATACTCATACAGCACTAAAATTTTATTTTACTGGAGTTTATC
AAGTGAAAACTTTGAAAATAACTATTAGGAGAAGAAGACAAGCAATGGACACTGGAGAAAATACTAACCCGTATTAAACATAAAGGGCTGTTGA
GAATATCCATCAGTAGATGTGAATGGCTTAAAACTCGAACTATATCCATATGGTAAATATAATTCAGCAATAATATAACAAACTTTTCTTTTTA
TTATTTTTTAAATTAGCAAACAAATGCTATTTATTTTTTAATATTTTTATTATTTAAGAAATTGATACAATGTATTTTTATCATACTCACTCCC
ACTCTTTTGTTTTATTGGTTAGTTTATTTCTTTACATTTCATATACTATCCCCCTTTCGTTTCCCCTCCATAAACACCCTATCCCTTCCCCTCT
CCCCCTGCCTCTATGAGGGTGCTCACCCACCCACCCACCCACCCATTCCTGTCTCCCTGCCCTGGCATTCTCCTCACTGGGGCATCAAGCCTTC
ATAGCGCCTCTTCTCTCAGTAATCCCTCACAAGCCCATGCTCTGCTACATGTGCAGCTGGAGGCATGGGTCCCTCCATGTATACTCTTTGGTTG
GTGGTTTAGTCCCTGGGAGCTCTGGGTGGCTCTGGTTGGTCGATATTATCGTTCTTCCTATGCCGTTGAAAACCCCTCCGCTTCTTCAGTCCTT
TCCCTAACTCCTCCACTGGGGACTCCATGCTCAGTCCAGTGGTTGGCTGCAAGCATCCCCATCTGTATTGGTCAGGCTCTGACAGAACCTCTCA
GGGGACATCTATACCAGGCTCCTGTCAGCAAGCACTTCTTGGCATCAGCAGTAGTGTCTTGGTTTCGTGTCTCCAGATCGGATGGATCACTACG
TAGGCCACACTCTGGATGGCCTTTCCTTCAGTCTCTGCTGCACTCTTTGTTCCTGAATTTCCTTTCGACAGGAGGAATTCTGGATTAATATTTT
TGAGGTGGATGGGTAACCCCATCCCTCAACCAGGGGTCATGCCTATCTACTGGATATGGTCTCTACAGGTTCTCTCTCCCCTTTGTTGAGTGTT
TTGGCTAATGTCCTCCCTGTTGGATCCTGCGAACCTCTTGGGTCTCTGGCATCTGGGACTTTCTAGTGGCTACCCATCCCCCCACTGCTAACCT
CCTTTCAAAATTCTCCCCATCTCTAAACTGCCCCCCTTTTCCTTCCCCCTGCTCTCTCTCTCCCAGGTCCCTCTCTCCCTCTACTTCCCAATAT
TATTTTCTTCCCTTCTGAGTAGGACTGTAGCATCCACACTTTGATGTGATCTTCTTTCTTCTTGGATTTCATATGATCTGTGAGTTGTAGTGTG
GGTAATCAGAGCTTCTTTTGGGTTAATATACACTTATCAGTGAGTGCATACCATGTGTGTTCTTTTGTGACTGCGTTAACCTCACTGTCCTGGC
TAGTTTTGTCTCAACTTGGCACACCTCGACTTATCACACAGAAAGGACCTTCAGTTGAGGAAATGCCTCCACAAGATCCAGCTGCAAGGCATTT
TCTCAATTAGTGATCAAGGGTGGAGGTCCCCTTGTGGGTGGTGCCATCTCTGGGCTCGTAGTCTTCGTTCTATAAGAGAGCAGGCTGAGCAAGC
CAGGGGAAGCAAGCAAGCCAGTAACATTCCTCCATGGCCTCTGCATCAGCTCCTGCTTGAGTTCCAGTCCTGACTTCCTTAGTGATGAACAGCA
GTATGGAAGTGTAAGCTGAATAAACTCTTTCCTCCCCAACCTGCTTCTTGGTCATGATGTTTGTGCAGGAATAGAAACCCTGACTAAGACACTC
ACTCAGGATGATACTTTCTAGTTCCATCCATTTGCCTATGAATTTCATGAAGTCATTGTTTTTAGTAGCTGAGTAGTAGTCCATTGTGTAAATG
GACCACATTTTCTGTATGTATTCTTCTGTCGAGGGACATCTGGGTTGTCCAGCTTCTGGCTATTATAAATAAGGCTGCTGTGAACATAGTGGGA
CATGTGGCCCTTGTTATATGTTGGGACATCTTTTCGGTATATGCCCAGCTCACTCTCACTCTTACCCCTAACTCCTCTCAGATAGACTCTTCCT
CCATCCCTCTCAAACTATGTGCCCTCCTCTTTCTTTTCTTTCATTCTTTTTTAATGAGCCCACCAAATTCAATTTCTGCTGCCCATTTACCCAT
GCTGTGTGGCCGTGTGGCCATCTACCGGAACTTGCAGTAGACTTGCCAGAGAATACACCCTTAAAGAAAACTGACTTTTTCCCTTCCTAGGAAG
TGACCAACTCTCCATACCTCCTCATTTATGGCTGCAGACCCCTTCTCTCTCTCTATGCTAGAATTTTGACTAGCTTGATCTTGTACAGACAACC
ACAACTGCTGAGTTCATGAGGATACCAGTCCTGCCATGTCCAGAAGATACTGTTTTGCTCTGATTCTCTGCAACTTCTGTCACTTAGAATCTTT
CTGTCCCCCTTCCACCACTTTCTCGGGCCTTTCAGGGATGTAACATAGCTGTCTCACTTGTTCCGAGCACCCTAGGACTCTTAGTCTCTGTACT
TCCACCAGTTGTAAGTTCTTGCATTTGGCCACCATTTGCTGCACAAAGAAACTTTTCTGATGATGTCTGAGAGCTGCCCTATTCTATGGGTAGA
GAGATCAAGACATACAAAGCAGTCCAGCCAAATGCATGTATCTGGAGGATATCATCCTTAGTGAGGTAACCCAACCACAAAAGAAGTCATTAGA
TATGCACTCACTGATAAATGGATATTAGCCCAGAAACATAGAACACCCAAAATACAATTTGCAAAACAAGGGAAGACCAACGGGTGGATACTTC
GTTCCTCCTTAGAATAGGGAACAAAATACCCATGAAACGACTTACAGACACAAACTTTGGAGCTAAGACGAAAAGATGGACTATCCAGAGACTA
CCCCACTGGGGATCCATCCCATAATCAGCCACCAAAGGGACCCTGCTAGAGCTGTCTTGTATGAGGCTATGCCAGTGCCTGGCAAATACAGAAG
TGGATGCTCACAGTCATCTATAAGATGGAACACACGGCCCCCAATCGACAACCTAGACAAAGCACCCAAGGACCTGAAGGGATCTGCAACCCTA
TAGGTGGAACAACAATATAAACTAATCAGTACCCCCAGAGCTCGTGTCTCTAGCTGCATATGTAGCAGAAGATGGCCTAGTAGGCCATCACTCG
GAAGAGAGGCCCCTTGGTATTGCAAACTTTATATGCCCCAGTACAGGGGAATGCCAGGGCCAAGAATCGGGAATGGGTGGGTAGGGGAGCAGGG
CGGGGGGAGGGTATAGCAAACCTTCACGATACCATTTGAAATTTGAAATGTATATAAAGAAAATATCTAATAGAAAGAAAGAAAGAAAGAAAGA
AAGAAAGAAAGAAAGAAAGAAAGAAGGAAGGAAGCAGTCCAGCTAGCACTGTAATAGTAGTAGGCTCACTCTGAGGCCTAGGAGTTCTTTAACC
ATGGGTTCTTGGTCAGATTTAGAGTACCAGTCATTTGTTTCCTCCTGGACAAAAGGACTTTAAGTCCATCAGAAGTGGTTGGCTACCCCCATAA
CCTTTGTGCCACTGTTGCACCCATGGGTGTGCCTTGCCGTGCCGGTTATCATTGTAACTGATAGGGTTCACAGATTGTCGATGCCATTTTCTTC
CAGCACCTACCATAGCACCTTCCACTACTGCGAAGGCTGACCAGATAGCAGAAAAACCCCTGCTCAGTACCAATCTGATTTCTTCATGCCCTGT
GACAAAATCTCTGTCCTATCTTCACAATAACGCTCTACCATCAAGTTCTGGTGAGCAGTCAAGACCAACAACAATGCCATGTGCTGTTGGAAGC
GTATCTGGCACATCTCTGTTCAACATCATGAGGAGAAGAATCCCAGGGGTGATCCTGGGCTTTTCATTTGGCATCTTATGGCTTCTGAGGCGAG
CATAGTCTCCTATGTGGCGTGCTTCCAGTTAAGCATCTTTATATGTTTATGTGCTTCGGAAGCTTATAAAGCAGTCGATTTCCGCATCGCCTTT
TCAAACATCCTTAGCGTTAGTTATCCTTGCCTCTTCTACCCTACTCTCCTGTCTCTCTGCTCACCTTAACCTCCCTCCCTTATACACTGTATTT
TCTTACATATATGTCATAAACATTGCTCACGTTTGATCCCACTGTACTCCTCCCCTTCCTTCCCCTACCTTCTTCCCTCCAAATAGCTCCCCTT
ATGTTTTCATACTATGTATGTATATATCTATTATGTATGTATTATCGTATATGTATATGTGTGTGTATATTTAATTCTAGGTCCTGCATATGAA
AGGAAATGAGATGCTTTATATTTCTCTTCTATTATTTTTACTTGTTCTCTTTTAATATAAACACCTTCTAATTTCATACTTAAATCTATATTCT
GCATATAAGAGAACATGTGCAACATTTGTCTTTCCAAGTCTGACCAATTTCACTTTCCAGTCCCACACATTTCCCTGCTAATAATATGACATTC
TTCTTTAATCTTGAATGACACTCTACTGTGTATTGTGCAACAGGTTTTCTTTATTAGTCTGTATTGTTGTCTGTATATTGCCATCTAGGCTCTT
CCTGCTTCCTGGCCATTTCAAAAGTTCTGTGGTAAGCACAGATGTGCATGCCAAGATAGACTCCTTCAGGGTATGCCCAAGAGTTGTACATCTG
GATCATAACCGTAGCCTACTTTGAGCTTTTTGACACACTCTGTGCTGGTTCCCATAATGGCTGTACTAGATTACATTTCATCTTAATCTAACTC
TACAACTCCAAAAGAGATGTTCTCAGAAAGCAGAGCGTAGATTTGGGGCTACTGCGAGGTGTAAGGGCTGCAGCAGACTTTGGGTGGTGATGCG
ATGTTCATGTTGATTGCACTGATGGCTTCCCATCAAAATGGCAATTGCATATTCCAAATATTGCACTGTCATGTACTAACGCTGTTTTCAGAAG
CTAGAAGGAAGAAGGACTTGCTACTGGAGTTTGTGCTGGTAGTAAGCATGACTGGAACCCAGCCTTTCGATTCACTTATGGACAGTGCATTGTA
AAATTACTCCCAGGGCTCCTTTTAAAGCAAAGAAGTCTTTGTAAAAAATAATATTACTTTCAATATCTCTTTTCTCATGGTTCCTATTGCAATA
TTGTTTCCTTAAAGATACATCACAGTTTGATATTGGAGACCAGAGTGACCACTGCACTGTATTGCTCATATTCCGCCATGAGGGGCAGAAGGCA
GATTCAGTAGCAGACTACACACAGTTTAGTGCATTCATTAAATCATATCTCATGGTTAAATATTTACTCTACTTAACATCTCTGCCAAATTTCA
GCTTAGGAACTTGCATGTGCTTTGTCAAAACAGCATTTCAAATACTTCAAAAATGATTTGTTCCTTTTTAACACCAATCTCAGGTTGTAATTAG
ACTGTACACACAAGCTCCATTTTAGCCTCCTAACGAAATCAAAACTATGTAGTGTTAAGGTGCTTCGTGAAAAGATGCAGGGTCAACAGATGCA
GTGAGCGGAAAGGAAGCAATAGGTTGCACCTCCCAGCCATCCCTGCCCTTCTTATGCCAACACAAAATGACTGTGGTGTTGCTGTAAATCTCCT
TCCAAATATGCCCCGGCAATGAAAACACGACACAGTTAATGCTGTGCACCTAGATTGGGCAGATCTACGGCTACACTACCAYCTTCTACAACTT
ATGAGACCCCTTAGAACTTGCAGTTTCTCCAGGCCATGTGCTAATCCTCCACTTTTCTTTGTCTTCTTCTACCTCCTCCTCTGCATCCTCTCCC
TCTTCCATTTTCTCCTTCTTATCTCCCACTCACCTTCTGCTCCACCTTCTCTTTTATCTGCCCAATCATCAGGTCTCCATTATTTTACAAATTA
AGGTGGGAAGCAGGTTTACAGGAAATCACCTGAGTGCTGACTCATTCCTTGTTCTCAGCCCCTCACACGACAATGGAATTAACACCAAATATAA
TTAGCCCCAGGGCTATGCACAACATTTCCCCCTTTCTGTCCAATTAAAAGACTATTTTATCTCAGATATAATTGAACATAACTATTATTATTTT
GTAATTTATAAAGTACAAGAGACCTAACCCAGTCCATCATCCTTGTTAATTAAACAGAACCTCTGTCATCTATTCTTACTTAAAAGACTTATAA
TTCTACATCTGGCTATGTCTTCGCTTTAGATTGAATGCCATCTGAACACCATCCTCTCAAAACTGTTCCTCTCCAAATAAAAAGCCTGAGTTGG
CTAGGAGACTATGTAGTTTTTCAACCTCATCAGAAATCTAAGAACCACTGATATTAACTGGAAATATATACAAACCCTAAAACGGCTTCCAAAA
CGTAGCCAATCCATAGAGACTGCTGATCACCTGGACAGTCCCTTACTTCAACACGCTGGAGCATCGGTCTTCAGCCTACTGGCCAGGGTCATCT
GATGGACTTAGAAAAACAGGAATATTAAGGACTAGCCTACTCTGTCTCGGCAGAATCAAGCTGTCCTAACCTGCAATTGCATCCTTTCTTTGGA
CAGTATTATGTCTGTAGATGAAATGAGCCAATTCTTGCCTAGTGGCTGTTTCACCACAACTGGAGTAACTCTAAGGTGCTCAGTTTCTTCTTTG
AATCCAAGACCGGGAAACCTGTCAGGAGCAGAATGGTCTCAATAACAAGTGAATAAATAACATTAAATGTCACATTCTGTGGACTTCTGATGTT
TCGGAAAACCAACTATCTATTTAAAGTAATGTAGACAGTTCTCTGTTAACTCCTCTCAGCCATTTCTAATTAAAATAACTGAGAACACCCTAAC
AATAAACTCAGAGCCATGAATTTCCTATTTCGCCCTTAACTCACAGGCTTAGACATCTCAGATCTGTTTATAACAGCAATAGAAGGACTCGGTT
CAAGCCTTGTACTTCAAAATTTTTAGAATCAAGGACCACCTATAATAACCACCCCCAGCCCCAAAACCTAGGAAACTGGGATGACGACTCTTCA
TAACTTCCTCAAGCTGATTATGGGCATTGAGATATTTTAAAGGGAGGGGGAAGGGTAACGAAAATGCGGGAGTTGGTTGGCCTTAAGAACACCC
CACCAATGATTGTATTAGTCTCTGATATCTGTGTCCTTACTGGATTCGGATGAGAAGCCAAGAAATCAGGAGTTAGTCAGGTCAGTTCAGCATG
TCTGACGATGAAATTCACCAAGGCTGAATATTCTGTCATATATAAATCTCCAAACAAAATTTTACTATCATCAAGATAACCTTTTTGCGGTCTC
CCGCTATCAAATCTGATCCATATAACTCTGCGAGGTCTAAATACATTATTCACCTTTTCCCCAAATCAGCATATCCTTCAGCCGGTAACCAGGA
AAACCTGTAGCTTGCCCTCTGTCCCAGAGGAACAATAATACAGCCACATTACTCAAAATCACACACATTTTAGCCTATTTAGGCCTTTCTAATT
TGTCATATCAGGATATTACCCCAAAATTCCCGTCGATCCCACTGTGTACAGAGTATGAGTCTCCTGCAGACTTTATTATACCATCTTTTAAACC
ATTGATTTAAAACCACACTTCTATTTTCCTTCCTTTTTCCTTTTCCTTTTCCTTTCCTTTTCTTTCCTCTTCCTTTCTTTTCTTTCCTTTCCTT
CCCACTTTTAAGGTGGGAAGCAGGTTTACAGGAAATCACCTGAGTGCTGACTCATTCCTTGTTCGCACCCCCTCACAGGAGAATCTAATTAACA
TCAATATAATTAGCCCCAGGGCTATCCACAACACTACGGATGCTTAGTAGTCCTGCTCCCTCTGCCAGGGCTCATGGGGACCTGGGAGCTTTGG
TGGTCTTTTTTCCTCCTTTGTGACTGAGCCAGCCTCTCTTTATTCCAGGGTTTGAACGGCATGTCTGTGGATGAGAAGCCTGGCTCCCCCATGT
ATGTATATGAGTCCACAGTCCACTGTGCCAACATCCTCCTGGGCCTCAATCACCAACGGAAGAAGGACATTCTCTGTGACGTGACGCTGATCGT
GGAGAGGAAGGAGTTCCGAGCCCACCGGGCTGTGCTGGCCGCATGCAGTGAATACTTCTGGCAAGCACTGGTTGGACAGACGAAAGATCACTTG
GTGGTCAGCTTGCCTGAGGAGGTACAGTGATTTGGTGTGGTTGTGGGGTTGTGGTGGAAGTTAGACGTGCACTGTGCCACGTCGATGGCCACGT
CACCCATGTCCACATCACGTCACCCACATGTGGGCTTGTAGCATAATCAGTTTCTGTTTGATGCACTTGAAGCCTCAGAGGCATGAATACAAAG
TGAGCGTGCTTCTTTAAGGGAACTGAGCCTCACCTGGGGCCCTGGCCCTCATAGTTTCTAGTTCACTTAAACTTCGGGTTTTGAATAGTTCAAC
CTCATTCTGATGCAGTCCTCTGTGTAGGATTTGAATCACTACTATTTCTTCTCTTTCAAATGGATCATTAGATTTCCCCTGAGAATAAAACAGC
GAAAGAATTCACAGGACCTTCCTTGCTTAGGAGAAAAACAAAGCGACCAGCAATTCTCACCATTAATGCAGTGAGATTCTAAGAAGTGTAAATT
TGCTTTTCTCATCAGGATTGAAAATATCCCCTACTCAGGTTTCTTAACTGCTTCAGTGAGAACAGTCATTTTGGTACTTTGGGGACATGAATAG
CAAGGCTTGTTCTGTCCAGAGATGACAGTCAAGTGGTTTGAGAGGCAAGGACCCAAAACATGGGTGTCACAATGGTCAACCATTAAACTGGTCA
CTCTTCAGACTGCTTATGATTCTTCAGTCATTCGTAGCCTCCTTATCTTCAGAAGACCGGGGTTAATGTGTGAATCCTTGAGCTTTGTGTGCAA
TGCAGAGTATTCTCTGCACAACACCAATATATGATACAACTGCAGAGCCTCTGGCAGCCGTTTGCAAGCTGTCTACAGCACACCATGCAGGAGC
ACACCTCACCTTCACGTCCTTGCGAATGAGACCCAGGGTCACCTCTCCACCTTCCTCACAGCAGTCTTCTATGATAAGTCAAAGACCAAAAATG
TAAGGAGTTAAAGATTACACTTACCACTCCATCCCGCCATCACCAACTCAGATGCCTTCAGAGACATACCCGTGACATTAACAACTCAATAGTG
CTTTGTAAAGAGTAGGGCGTGATGGAGTGAGCAGCTAGACCACCAAAAACACGTTCAAGCCTAACACGAGAGAAACACAAAGGGGGCTGGAGAT
GCAGCTTGGATAAATTGCCTAGAGCGTGGTCCCCAGCAATAAAAACAAGACGCATGGGATGGAAGAGACACCAGATTTCAGTCCCTTGCTACAC
AGCATTTGTGACTACCCCCGCAGAGAGCAGAAGAATGGCCAGCTGATGAACCGCTGACGTTTGAAACATCTTCACATAGGCACCCTGTCATAGT
CACAACCAACTGCTCCATTCCCATTTCCTACACAGCCGTGAGAGAASGAAAAGAATAASTCATTTCAAGATTAGGTAGCCAAGATCAAGTCTTC
GATCTAGTAAGAAGAGAAGGAGAGCGAGCAATGGCAGCTGGGTCCTTAGCAGGATGGGCGGTTCCCATCTGCACTGGATGGAGGCATGGCCGTC
TGTAAACTGCTCCTCCGCTTCGCAGCTGAGGTAGTGATGTAATGCTGCAAACCCAGCCCATCCTGCAGCAAGTGCTGCTCAGACACATGTCCTT
CTTCTCCAATGGGAACAAAGACGACATCCTCCTCACTCAGTCCAACTCTAGAATTCTCTGGAAATAGTTTCATGACATCTAGTTATACAAAGTA
GTCTTAGAGGAGGTGACACATGTTGCCTACTTATTTCTGTTGTGTCTTGGAGAGCCATTTCATATGTGTATATGTATTACTAATTTCCAAAACT
TAGATATTAAAGCAGATACACAAACGAAGTCATACTTTATGTATCGAGTCTTCCCTCAATGACTGTGTTAGCCCTACCTCATACTTCCTAGAAA
AGAAGACCTAAGCCCAAGCCTGTTCTCAATTCTAACCTGGATCCTGATCTACTAATCTCTCCGACCCTATGTGTCAGTGTGTGCATGTGTGTGT
GTTTGGATTTTGGGTTTTTTTCCCCACACCAGATTTCTCTTTGTAGCCTTAGCTGTGCTAGACCAGGCTGGTCTCAAACTCTCAGAAATCTGCC
TCCTAAGTGCTCAGCCATCATCTTAATTTCAAGAAGCCCTAAAATTCTGAAGCCCTTAACCTATAATCAAAATACAGTACCCATCCAGTAAAAT
GTCAAGGAAACCAGCGTATAAATAAATCTTACTAAGCCTCAAGTTTCTTCTCAAGTCAA
MOUSE SEQUENCE—mRNA
CGAGCTGCCATGTGATGCCCATCCCCTCCGCCTGCTCTCAGTGACCCCAGGACCGCCCACCACGCCGGCGGCCGGGAGGGGGCTGCGGGCTGGG
AAAACACGGGCAGGACCGGCGCAGGGGGACAAAAGTTCCCGGGAAGCTCTCTGCTTCCAGAAGTGAGCCTTCTGAAACAAGAAGGACCTTCAGG
AGCTGCTCCACACTGTGACATGTCTGATCCCTTGTGCCCGTCCCTGCAGCATGAACAAGGTGGACACTCACTGATCTATCACAAGATTGCCTCA
CTCAGTTTCGGCGTCCATCTCTGAATGCATTGCTGTAGCCTTCTCATCTCTTCCTCTGCCCAGTTCCCAGCATCCTAAGACTCGAAGCCAGGAC
AGGACCTGGGGAAATTATATGGTTTGAACGCCATGTCTGTGGATGAGAAGCCTGGCTCCCCCATGTATGTATATGAGTCCACAGTCCACTGTGC
CAACATCCTCCTGGGCCTCAATGACCAACGGAAGAAGGACATTCTCTGTGACGTGACGCTGATCGTGGAGAGGAAGGAGTTCCGAGCCCACCGG
GCTGTGCTGGCCGCATGCAGTGAATACTTCTGGCAAGCACTGGTTGGACAGACGAAAGATGACTTGGTGGTCAGCTTGCCTGAGGAGGTCACGG
CCAGGGGCTTTGGGCCACTGCTACAGTTTGCCTACACTGCCAAGCTGTTACTCAGCAGAGAGAACATCCGGGAGGTCATCCGCTGTCCTGAGTT
CCTGCGCATGCACAATCTGGAGGACTCCTGCTTCAGCTTCCTGCAGACCCAGCTCCTGAACAGGGAGGATGGCCTGTTTGTTTGCAGAAAGGAC
AGTGCGTGCCAGCCCCCCCAAGAGGACCATGGGAATTCTGCAGGAGAAGAGGAGGAAGAAGAGGAAACAATGGACTCAGAGACAGCTAGGATGG
CTTGCGCCACAGACCAGATGCTCCCAGACCCCATCAGCTTTGAGGCCACTCCCATCCCAGTAGCAGAGAAAGAAGAAGCCCTGCTCCCTGAGTC
TGAGGTACCCACAGACACCAAGGAAAACTCAGAAAAGGGTGCATTGACCCAGTACCCTAGATATAAGAAGTACCAGCTCGCGTGTACCAAGAAT
GTCTACAGTGCACCCTCACACGGTACCTCAGGGTTTGCGAGCACATTCAGTGAAGACAGTCCTGGCAATAGCCTCAAGCCGGGGCTCCCCATCG
GACAGATTAAAAGTGAGCCACCCAGTGAGGAGACCGAGGAGCAGAGCATCACCCTCTGCTTGTCCGGAGATGAGACCGACATCAAAGACAGACC
AGGAGATGTCGAGATGGACCGAAAACAACCCAGCCCTGCCCGTACCCCCAGTACCCGTACTCGAGCCGCCTGCCTGCACAGGTCCAGGAGTGTG
TCCTCGCCTTCCTGCCTGCGCTCTCTGTTCGGTATAACGAAAGGTGTGGAGTCCACTGGCCTGCCCAGTACATCTCAGCAACCCTTAGTCAGGA
GCTCAGCGTGCCCTTTCAACAAGCGGATCTCTCAGGGTGACCTTAAAACTGATTACACCCCTTTGGCAGGGAATTACGGACAGCCCCACGTGGG
CCAGAAGCATGTCTCCAACTTCGCAATGGCGTCGCCACTCACGGCGCCCGGGCCGGAAACTCTCTCTGAGTTCTCCTCTTCGCCCTGTTCCCAG
GGAGCCAGATTCCTTGCCACGGAACATCAGGAACCAGGGCTGATCGGAGATGGAATGTACAACCAAGTCCGACCCCAGATTAAATGTGAGCAGT
CTTACGGAACCAATTCCAGTGACGAGTCTGGATCATTCTCGGAAGCAGACAGTGAGTCGTGTCCTCTGCAGGACAGCGGCCAGGAGGTTAAACT
TCCTTTTCCTGTCGATCAGATCACAGACCTTCCCAGGAACGACTTCCAGATGATGATCAAAATGCACAAGCTAACCTCAGAACAGTTGGAGTTC
ATCCACGACATCCGGACGCGGAGTAACAACCGCATTGCAGCCCACCGCTGTCGAAAGAGGAAGCTGGACTGTATTCAGAACTTGGAATGTGAAA
TACGSAAACTGGTGTGCGAGAAGGAAAAACTGTTGTCGGAGAGGAATCACCTGAAGGCGTGCATGGGAGAGCTCCTGGACAACTTTTCCTGCCT
CTCCCAGGAGGTCTGTCGAGACATCCAGAGTCCAGAGCAGATCCAGGCCCTGCACCGCTACTGCCCTGTCCTCATACCCATGGATCTCCCTGGA
GCCAGTGTTAACCCTCCTCCTGTGGGTGTTGAGCAGAGCCTTGCACCCTCCCCGTGTGCAGTAGGAGGAAGTGTACCCTGCTCCCTCGAGCCAG
GGGCGGCTCCCCCCGGGCTCCCCTGGGTGCCCAGCAACACCTCTGAGAATTGTACCTCTCGGAGGACGTTGGAACGCAGTCACCCTGGAACATT
CTCAGAACGAGGACCTCCCCTAGAAGCCAGGAGCCAGTCTGTGACGGTGGACTTCTGCCAGGAAATGACTGAGAAGTGTACAACGGACGAACAG
CCCAGGAAAGATTATGCCTAGCCGGTGTGATCAGCTCTCCCCTACCCACCACCCTACCCCCGCGTCTTCACACCTCTCTCATCTGGGTGTTCTT
CTGCCAGCCAGTGGCGCTGTTCATCTGTGTCTTGAAGAACCAAGAGCGAATTTGGTGCACACTACAGCAGTCTTAGCACCAATACTGTTTCGAA
GTATTCCCTCCTCTCTCTAAGCAGGACTGCTAGTTACCTTCATAATGGTGCTACCCCTTGCCCTGGCAAGGAAGACAACAGTGTCGACACTGTC
TGTCTGTGGGTTCATTGCAAACTTCACAGGGATAGACTACACCTCTAGGACCCAACCACGGATTTTTTTTTTCTCAGTGGCCCATGTCACAAAC
CCTATCTCAGGAATTTCTTCTGAATGTTCAATTTTTTTCATTGAAGACAGCTTCTATACACATCAAAGTTTTATAGCTATACTGTGTACATAGT
ATATATAATATATATGAAATATATATCCATATGCAAAAAGTCCTGCATGCCTCAATTTTCTCATCCCTGAAACTGGAAGCTTCACTTATCATTT
ACAAACAGGTTCCAACATTCCTCTTTTTGTGTCTGGTGCCAGAACTGGTTTGGAAGCTGTTAACATGGCTGTTTTGCTTGCTGCACAATTCGGT
TTCCATCTGTGCTTATTTACAGACAAAATTCAATGTTGGGAGATCCTTCTCAAGGTTCAATCTCACACCTTTTACTTTCGTTGGTTTGGTTTTG
CTGCCGCCGAACGGTGTCAGGTGAGCACCGTGAGTCCGCTCTTCTCCCCTTGTGTTTTCCCCTCGTCTCTGCGGATACTGTACAGCAATGGTCA
ACTTTGCCACTTGCACTGAGTTTTGAGTCAAACCTATTTTCTTAAATGAAGTTGTAACTTCGGTATAACTCAAGTATATTTTTTTTTTCGTTTC
TTTTTCTTTTTGGAGACAGGGTCTCACTATGTAGCCCTAGGTGGCCTGAAACTTGCTAGGTAGACCGGGCTAGCCTTGAACTTAAAGAGATCCA
TCTGCCTCTCCCTCTGCCTCTGGAGTGCTGGGATAAAGAGTATGCACCACCATGCCTGGCAGTCCGGGAATGCCTACCCGTTCGCCACCATGAC
ATTAGGTAGCAAAGCAGCCGGAATCGCTCCTCTCTGGCGGCTGAGGGTCTCACTGAGGAACTGAACCAGCAGATGTTCTACACCTTCTGTGCTG
TAGGAAGAGAACTCACAGCTGCTTCCAAGGCTTTGACACTGTGTGCAGGGCTAGAGGCCAATGGTATAGGAGCGGCCGGTAGGGTTGTGAATAG
AAGAACAGACGGGGCCCGTCACCGCCCACTCGTACCACACTTTCTTGCAATTCCTCCATCGCCAGAAACCCACACAGATCTTCTGGCCTTCGTG
CACCGTGATGGGCTGCTTAATGGGGAAGAAGATGGCGAACCATGAGAACATTCCAGCACAGTGAGTCTCTGGCCGGATACTCAGAGTGATGTCC
CGGTAAAGCACAGTCTCAAAGTAGCCTGCGAAGCCATGAAGCACCGTGTTCACCTCCACAGGAAACTCCAGGGTACAGTAGCGGTTGTTGTCAA
TCATAGGATCTCGGTTGGGATGGCTCAAGGTAAAGCAGGGCTTAGGAGCAGAGAGCTGGTGGAAGTTGTCCAACCGAACCACATAAGGCATCTC
AAACTGTGCCTCACGATCGCGGTCCTTTTCCCGACAGGCACGGACCTCGTTGTACAGCTTAGAGGAAGAAATGGGAGCCAGGAAGGAGGTGTAT
TCTCCAGGGATGCTCACGCCATCATCTTTCAGGAAGTGCTGTGCTCCATCCAGACACTCAGGTGACAGCTCGTTGTCGGCAAAGGAACCCAGAA
GCTCACTGACAATAATGTCAGCTTTCTCCGGAGCCACCCATTCCCGCATGTCTGATGAGACAACTGTCACCTGGCTCCCCCATTCTTCAAACTG
CCAGTTCTCTAGCGTCACCACAGCATTGGGGTTCTTCTCCACAGCATACAGCCTGATCCGCCGCTCGGCCTGTTTGGCTGCCCGAAAAGACGCA
TTCACAAGAGGACCCCGGCCTGCACCCAGCACCATAAGTACCTGGACATTGGTCTCCTTTTCTTCTTCTGGTACTCGGTCTAGCAAACATTTAT
AAATAGCCTGCTGATATTGAGAGTATTTGATGGGGTCCTTTTCAAACACTTCATATGTCTGAGATTCCAGATTGTCCATCAGAGGCTGGAGTGG
GGACTGCAGATAGTCTTCATAGCCTTTGGCAAAAAGCTCATAGGCATTGGGTGGAGGGCGATTTTCGCTTAAGTATTCCAAGTACTGGAGGTAG
GAACAGAACTCCTTCTCTGAGTGGTGGTTGGTTCCCGTGATGATAAACTGCACTTCCAACTTGAGGAGCCGGAAGATCAGCCTCTGCTGCACCT
TAGAAAGAA
MOUSE SEQUENCE—CODING
ATGTCTGTGGATGAGAAGCCTGGCTCCCCCATGTATGTATATGAGTCCACAGTCCACTGTGCCAACATCCTCCTGGGCCTCAATGACCAACGGA
AGAAGGACATTCTCTGTGACGTGACGCTGATCGTGGAGAGGAAGGAGTTCCGAGCCCACCGGCCTGTGCTGGCCGCATGCACTGAATACTTCTG
GCAAGCACTGGTTGGACAGACGAAAGATGACTTGGTGGTCAGCTTGCCTGAGGAGGTCACGGCCAGGGGCTTTGGGCCACTGCTACAGTTTGCC
TACACTGCCAAGCTGTTACTCAGCAGAGAGAACATCCGGGAGGTCATCCGCTGTGCTGAGTTCCTGCGCATGCACAATCTGGAGGACTCCTGCT
TCAGCTTCCTGCAGACCCAGCTCCTGAACAGGGAGGATGGCCTGTTTGTTTGCAGAAAGGACAGTGCGTGCCAGCGCCCCCAAGAGGACCATGG
GAATTCTGCAGGAGAAGAGGAGGAAGAAGAGGAAACAATGGACTCAGAGACAGCTAGGATGGCTTGCGCCACAGACCAGATGCTCCCAGACCCC
ATCAGCTTTGAGGCCACTGCCATCCCAGTAGCAGAGAAAGAAGAAGCCCTGCTCCCTGAGTCTGAGGTACCCACAGACACCAACGAAAACTCAG
AAAAGGGTGCATTGACCCAGTACCCTAGATATAAGAAGTACCAGCTCGCGTGTACCAAGAATGTCTACAGTGCACCCTCACACGGTACCTCAGG
GTTTGCGAGCACATTCAGTGAASACAGTCCTGGCAATAGCCTCAAGCCGGGGCTCCCCATGGGACAGATTAAAAGTGAGCCACCCASTGAGGAG
ACCGAGGAGGAGAGCATCACCCTCTGCTTGTCCGGAGATGAGACCGACATCAAAGACAGACCAGGAGATGTCGAGATGGACCGAAAACAACCCA
GCCCTGCCCGTACCCCCAGTACCCGTACTGGASCCGCCTGCCTGGACAGGTCCAGGAGTSTGTCCTCGCCTTCCTGCCTGCGGTCTCTGTTCGG
TATAACGAAAGGTGTGGAGTCCACTGSCCTGCCCAGTACATCTCAGCAACCCTTAGTCAGGASCTCAGCGTGCCCTTTCAACAAGGGGAACTCT
CAGGGTGACCTTAAAACTGATTACACCCCTTTGGCAGGGAATTACGGACAGCCCCACGTGGGCCAGAAGGATGTGTCCAACTTCGCAATGGGGT
CGCCACTCAGGGGGCCCGGGCCGGAAACTCTCTGTGAGTTCTCCTCTTCGCCCTGTTCCCAGGGAGCCAGATTCCTTGCCACGGAACATCAGGA
ACCAGGGCTGATGGGAGATGGAATGTACAACCAAGTCCGACCCCAGATTAAATGTGAGCAGTCTTACGGAACCAATTCCAGTGACGAGTCTGGA
TCATTCTCGGAAGCAGACAGTGAGTCGTGTCCTGTGCAGGACAGGGGCCAGGAGGTTAAACTTCCTTTTCCTGTCGATCAGATCACAGACCTTC
CCAGGAACGACTTCCAGATGATGATCAAAATGCACAAGCTAACCTCASAACAGTTGGAGTTCATCCACGACATCCGGAGGCGGAGTAAGAACCG
CATTGCAGCCCAGCGCTGTCGAAAGAGGAAGCTGGACTGTATTCAGAACTTGGAATGTGAAATACGGAAACTGGTGTGCGAGAAGGAAAAACAG
TTGTCGGAGAGGAATCACCTGAAGGCGTGCATGGGAGAGCTCCTGGACAACTTTTCCTGCCTCTCCCAGGAGGTCTGTCGAGACATCCAGAGTC
CAGAGCAGATCCAGGCCCTGCACCGCTACTGCCCTGTCCTCATACCCATGSATCTCCCTGGAGCCAGTGTTAACCCTCCTCCTGTGGGTGTTGA
GCAGAGCCTTGCACCCTCCCCGTGTGCAGTAGGAGGAAGTGTACCCTGCTGCCTGGAGCCAGGGGCGGCTCCCCCCGGGCTCCCCTGGGTGCCC
AGCAACACCTCTGAGAATTGTACCTCTGGGAGGAGGTTGGAAGGCAGTGACCCTCGAACATTCTCAGAACGAGSACCTCCCCTAGAAGCCAGGA
GCCAGTCTGTGACGGTGGACTTCTGCCAGGAAATGACTGAGAAGTGTACAACGGACGAACAGCCCAGGAAAGATTATGCCTAG
HUMAN SEQUENCE—GENOMIC
ATGATGTGTACCTGTAGTTCGAGCTACTTGCGAGAAAGAGGCAGAAGAATCACTCGAGGCCAAGAGTTTGAGGCTGTGGTAAGCCATAATTGTG
CCACTGCATTCCAGCCTGGGCAACAGAGTGAGACCTTGTCATTCATAGATAGATAGATAGATAGATAGATAGATAGATAGATAGATAGATAGAT
AGACAGGAAAAGGAATGTAATGGACATTTCTCCAAAGAAGATATACAACTGGCCAGCAAGCACACACACAAAATTCTCAATATCACTAATCATT
AGGGAAATGCAAATCAAAACCACAATGAGATACCACCTTACACCCATAAGGATGCCAACTATTAAAAAAAAACTAACGTGAAAAAAAATAAAAT
AAAAAGTATTGGTGATGATGTGGAGAAACTGATACCTAGAGTAGTCAAATTCATAGATATAAAAAGTTAAAAAGTGGTTTTCAGGGSTTGGGGA
GTTGTTTCATGTTTTGCAAGATGAAAGGCATTCTGGAGATTGGTTGCACAATATGAATGTACTCAACTCTCCTGAACTGTACATTTAAAAATGA
TTAAGATGATACATTTTATATTATGTGGATTTCACCATAATTTAAAAAGTAAATTTTAAAAAAGAGGGGGTGGATATAATTATAATAATTGCTG
CAAAGGATTTAGGTCCAAGGTACCTATAAAGAGGATCTGACCTGGGTAGTGGTGAAGAAAGCTCCACTTAGCTGAGAGCAGAATGAGTAGGAGT
TTGCTGGGGGGAAGAAAAGAGGTAGAAAGAACAATATATGCAAAATCCTTGTGAGGTGAGTGATCTTCATGCACATGGGATGCTTTCTTAGGTA
TCAACTAAGTATAAACCTTTATTTTCACTTGACTATTTCTGTTTTTAAAAATGGAGACATAACTTATCAACACAGATTGCAAAAACCTTAAGTG
AACAGCTCAGTAAATTTAGTAAAACACTTTSAATCCAGTAACAGTCAAAAAAGGTGCAGTTGTGCTTCCTTTATCCCTTCCTGCCCCTTCCCCT
AATGCTTCCCTGGGCTGAGGATATGTCAGGGGGAGCTTTCCATACTGAAGGAGACTAAAGAGCTAGCACAACTAACTGTACCCCATGATCCTGA
CTCAAACTTTGCCCTAGAAAGGAAAAAAGATATTGTTGGTACAGTTGGTGAAATTTGAATAGGGTCTCTGGATTGAGTGGTATTGTTATACTGA
TGCTTACTTTCTGATTTGGATGTTGTATTGTGGTTATGTACCATTATTCCTTTTGAAAATGTGCACTGATGCATTTAGCAGCTGATGGAGCATC
ATTGTTGACAACTTACTTTCAAATTATTCAGAAAGAGAATAATGGTAATGGGTGTTTATATTAGATATTTCTGCAGGCATACGGAGAGAGAAAG
AGAGAACAAATGTGTTAAAATATTAACAATTGGGATATCTGCATGAAGGAGATATGGTAGTTCTGTGTATTACTCTTGAAACTTTTCCTAAAGT
TTAACATTATTCCAAGATCAATTATTTTTAAAAGATACTGTCTATGATGGCAATGGACTCACAGGTGAGGCAGCAGGTAAGAGAAGTGAGATTA
GACATAAAGATAAACATTTTATCTGTCACTGAATTTGAAAAAGCAAAGTGTCAGAGAAAAGAATTAGTGTTCACTTAGCGCTTGCCAGAATGAA
GTAGGCTATCCTGCAAATAACCCACTTATGGTTATCATAAATTAATAAGCATTCCGATTCAGCATCTGCTTTCATTAGCATAAGTTGGTCATCA
GGCTGGGAGCACGGGTTCCTCCTCTAGACAATGGCCCAGCCACCAATCTTAAGACACCTTATATCCCAACCCACTTCCCTAGACCATCCTTTCC
AGCTTTAGAAAATTAAGCACCAAGGAAATGTACCAATAGAAGCATGCCTGTTGACAAGACCTGGGAAGAGCTGTAAATCCAGAGGGTGCTGAAG
TGCAGCTGGAACAAACACAGCACACCTCCATGTTTTCAGCCTTCTGCAAAATCACCAAAGGCTCAATTCAAGTTGGGGCACGAAACACTGGGTT
TCCACACACACCTAACAATCGAAAGGCAGAGAAATTAGCTCCTCAGTGTGTGGTACTGTGGGAGACCTACTTAGAGAGCACCTGGGGAAAATGG
AGAAACCGATGACACCTAAATGTCTGTCATTCACATAAGCACTATGCCGGATGCACACACGGGGCACAGAACAGCTGTGCTGACCAGCACATTG
CTGCAACCACTGTTCAGGTTGGATGCCCTTGCATAATTATATCACATAAACAGATTTGTGAAAAACAGATTAGAAACTGTAGGTGCCATCTTAC
ATATTCTTTGGGGAAATTGCTATAGGGTTAGTGAACTCAACTTTCTGTGTCTCTCAAAAACTCATAAGCTGAGTTGAGAAATATAATTCAATCG
CAAGTATATTTTTATCAAAATAAAAGCATCAAGGCTTTCTTTTCACAGTAATTTAAAAATTGTCAGATTACTTCTGTCTACGACCAAACTTTTC
CAAGTTTCTTTAAGCAAAGATGTAATAAAAGCTAAAACTCAGACCACTGATCAAAAACATCTTTTAGAGGTGGGCAGAATAAAGAGATGGACAT
TTAGTTAATGGAGTAGTTTAGAAAAAACTAGGCATCACAAACCCTGAGGGTAAAAAAAGGGAAGCATTGTTAGAGCTTTTTTTAAAAAAAAAAA
AAAAAAGGGAAAAAAAGAAAGTAATCTTGTGATTATTATAAATTTTATTATTATTTTGCTTACCTGAGCCTGACCTAATCACCTCCTTCCCTGT
TTCCCAGGTAAAAAAGAAAAATTAAGCTCTTACCCGAAATTTCATGTCTCTTGGCCCTACATCTTATCCTGAAGCTTCAAAATCTTTGCATCAT
CCCTTTCTTGGATGTCCCTGTAATCCAGACAGTTTTCAGAGGCGAGATCTGAGTGATCCTCCCCCGTCCTGCAAATTCTGTTTAATGCTCATGT
TCTGCCTTTCTTCCACCTACCCATGTTTACCCCTGCCCCAGAGTTCTGCATTCTTGCCATTTTTTTCCTCTACATCAGTGCTCCTTCTGTTATC
TGCTCCATTACCTTACCTTCTACCTCTTCCTTCACTCAGATTCCTACCTTCCTGCCTTTCTCCATCTTTCCAGGCCCCCACTTCTCTCATTCAT
TCCCCCCCTCCTCTGCAGTGGGTCTAGAAGCTCCTTGCTGTCATATCACACAGCCCCCATCCCACCAGACCTTTCTTAATCATGGGATGCTATT
CCCAGGCCCCTCATTGAAATGCCCTTTTCCTTACCTTCCTCAACTTCCCAGTCTCTGCTTGATTTTCTACCTTCCTGTTGGCTCCATTCCTGTC
TTTTTCATAAACTCCTTGTTCTGTGCCCACATCCATCCCCAACCCAGCTATTCCTCAAACTTCAGCATTGAGGAAGTCAAACTTCCCTCCTTTT
GACTCCACCCTTTACCAGAGTGATCTTAGCCTCCTCCTGCCTCCCAGGGCCAGTTCCCTCCTAGTCACACAGGCTAGAGGTCTCCATTCCCTCT
TTCTCTTCTTCCTCCCTCTCCGTCTCCTCCCACAAGCAAATGACCCCTAAACCATAAGCGCTTCCCAGCCCTGCTCACTGCCTTAACATAGGCT
GTAGGACTTGAGCTACTGTCTTATAGTCTCCAGCCTCTTCAGGCCCCTTCATATGGCCAATGCTGTGTTCTTTCCAAAACCAGATCTGATTATG
CCTCTTTGGCAACTGGGGATCACTCATCGGCTTTCTACCCATGCAGCCAGGCTTTTCAGTGTTTCTGGGGAAATTAGAAGATAAACTCGGAGCA
TTTTCCTCAAGTACATCCTTCAGTACTTTGATTTTGGGTGTGGGAGAGGAACTTTTAGTCCTGATCGTCACAAATTGTTAAACTTTTTTAGTAA
AATCAACCCATGTACTTCAAAGAGAACAAAAGAAGTTCAGGTGATTTTTTAACATGTAACAGGAAACTGCAAAGACATTTTATGCTTTGAAAAG
GCAATTTGGGGCCATGTTCTCAGATCCCTAGGAAGTTAGCATTCCATTTTCTCTGCCATTCTGAGCACTTCACTGGAAAAGTTCAATGAGAATT
TTGATCTGTGGATAAAATTACACCTTCATGCTGATTTCCCATGTCTACCTTTCCAGTCCCATATTCCACCACTGGCTGTCGCTGCTAACCATCT
CAATTCCTGCACTTTCGCTACTCCAATTTTCCTGCCTGGAATCACCTTCCAACATTTTCTTTTCACAAAATCTCATCCTTCAGAATCCAGCTCT
GTTGTCACCTCCTCCAGGAAGCCATCCCACATTCTCCTAAGCAAACTCACTTGCCAGAAGACTTTTGTCCTATTCAGGCCCTTAGCATGGTGTG
CTGTGAGGACTTCTTCACCGGTTCACCTTCCCCACTAGTCCAGGAGCCCTGACTTATGCAGTGTTTTATCTCCAAAAGTTAGAATAGTGCTAGT
ACATAGGTTGTGTATGCTCGAGGTCTGTCTCGAAAAAAAGAAAACAGGCAAGTAACAGGTACAAGGTCTTCCCCATTGAGAGGGGAAAGAAGGA
AAAATCTACATTTTGAAAAAATGAGTCATGATCACAATGGCATTTTCTCACCATGGTACTTCAGTACTCCTTGCTAGCCATGACAGCAGGCACA
ATCCCCCATACCTTCCACTTCTGACCTATCCTTCTTGACATCACAACCTCTCACTCTTTCCCAACACTGCAAGATATGAATGCTATAGTCAGAT
GAAAGTGTAACCTCAAGGGGTCCTTCTGCATTAAAAGTCCAGCCACCTTCCCTCCTCTCTGACTCCTTCCTGGCCTTGAGGTTCAACTGAAATT
CCACCTTCTCACTGAAACCTTTTCTGGCTACTTTAACCTTCATAATTTGTTTTTCCTCTTAACATCTACAATACTTAAGTTCTTGTTAGAATTT
AATTATGCATATTTTGAATTTTACAACATTTATTGGCTACCTTTATTACAGAAGTAATATATTTATATTACAGAAAATTTTTTTAAAAAGCAAA
AGTAAAACCACTTATAGGCTCTTTATTTCAAGAGAACCACTTGAACACTGTGCATATAGCCTTCCAGATTTTTCCTATGCATGCACTGGCAATT
TTTTTTCACAAATAGGATCCTATTGTAAATAGTTATACAAATTATTCTTAAACTATACTAACAAAATATTAATATATCAATATACATAAATTTT
CATCACTAATTTTTTTCATTATTATTATTATTTTTGGTAGAGATGGGGTTTCACTATGTTCTCCAGGCTGGTCTCAAACTCCTGAGCTCAAGAA
ATTTGCCTCCTTCAGCCTCTCAAAGTGCTGGGATTATAGGCATGAGCCACCGTGCCCAGCCACCAATATTATTTTTGATGGATCCATGATATTC
ATATTTTATATATGAACCACCATTTATTTAGCCAATCATTTCCTGTAAGGCATTTGGATTTTTCCATTTTCTTTTTGCTATCATACAAAAAAGA
TCTGTACATTCTTAATTTTTTCATTAGTATAAATTATAACACATACAATTTCTAAATAATTTTAAATATTAATATATAAAAGTAATAAAATCAA
ATACAAAATTACATTTTTCAATTGTTTCATGTATATTAGTTTTGACTCCTTATGTAGATTTAAGTTCCTTAAGGATACAGTCCAATGTTTCTTA
ATTTTTCACCTCAACATGTAACAGATGCTCAGTAGTGTATGTTGAAAAATTAGTTTATTTCATATACCATATTTTACTAGTTTATTTCATATAA
GCTTTTAGATAATAGTGGCATCAATTCTTTACTATAGAAGAAGATCACTATAGATAGTAATATAAGAACATCACTTTCTTTTAGGGACTATTTC
CAAAGTCCTTCTTAGGAACTGAGGCAATCTTTGAGGACATGAAGATGGGACAGATAAGAGATATACAGTTTGAAGGAAAAAGGCTGCCAGCCAA
ATTTAACAATAGCTAGAAGCAAAATTTTTGCTTTTCTGGTTTCGATTGCCCTGATCATTTTTCAAAACTTGTGTCAGATTTTGTACAATGAGAA
ATCTCTTCTTGGCAGTGGTGTGTCCAGAAAGCCCCCACGAGTGGGCCCCCAAGGTCCTCTGTGCCGTCCAGCAATTCTGCCACATCCCTGGCAC
TGGTTCACATCTTTTCTCTATCCCCTAAATCTATCCTTCCCTCCTCCACTATCAGTGAACGGTCTATCTTCTTACTCCACTAAGAAAACAGAAT
TCCTCAGACAACGGCTTCCCATACTTCCTACCATGATATTCCAGCCTCCCTGCACTTCCCCACCTGTGCTGCCCTTCCTTCTGTTGGTTGGGAT
GAACTGGCCCCTTTCTCTCTCAAGCAACCTTCTCAGCTTCCTTGTGGGGATCTTTTTTTCTTCATCTTCCTGACCTCTAAATGTCGGAGTCCCC
CAGGTCTTAAACCTTAGACTTCTTCTCTTTGCTCCTCTTCTCCACCCATGTTTCCAGTTGCTGTGTTCATGACACACAGATTTCTATCTCTAGT
CTGGACTTCTCCCCTAAATTCTAGATTCCCATGTCTGATTACCTGCTCACCTTTTCCACTTAGATGTCTGATAGACATCTGAAATGTACTATAT
CCAAAACCAAATGTTTCACCATTTCCCCCATACCTGCTCCGCCTGGATAAATGAACTCTCCATTAATAAGCTGTTCAGTCCAAAATTCAGAATT
TCCTTTGACTCTTCTCTCATATCCTACAATGGAATCATCAACAAATCTTGTCCGCTTTATCCTGAAAATATATCTGGATTCCATTTATGGCTGT
CATTCTAGCATTCTATTAATAGTTTAAATCACCATCACCTCTCTAATGACTTATCTCAATAGCCTCCTACTTGGTCTCTCTGCTTCCATTCCTG
TACTTCCCGTGACCCAGCAGAGTGATCCTTTAGGAGTTGACGCCAGGACATGTTCCTTGTCTGCAGAAACCCCTACAGCATCCCACTCCATTCA
CTTTTGTCAGATTTCATTCGATGTTAAATTTGCCCTGCCATGCCTTATTCAAGGTACCACATTTGAAATTCTGTCTACTGTTCTGCCTAAGAAA
AAATAGAATATTTTCTCAACATCATCAAATCCTGTACAGGGTGCTGGACAGCAAGAAAATCAAAGGCTCAkCAACTGCCCATGTCTTTAGTCCC
TCTTCCTTTCCTCATCTGTTTCCTGTATATTTTAATTTTTCCTGTTTTATTTTCTACAATCAAGAAGATTTCTATTATAGAAATAAAGTTTATT
TGTTTCAATGCCATGATACAGATTTATTCTCCCTGGAAATATCATGCTTAGAAATGAATCACAGACAAAGCCCATGCTCTGGATCAACACACAT
GTCTAATTATGTAGGAAAAAAATGTATATCAGGAAAGGTTGAATGCTCCAGGCATGAGAAATAAATTGACCTCCACTCAACTTACTGCCATCAT
TATACAGAGGGAAGGACCTGGATCTGTTTCACATCATAAACGCCTAAATTGCCACAGCAGCACAAGCCCAGGAGTGGCCATGCAAACTCATGAT
CGAGGTGCTAAGGCTGTCCACAGCCCAACCTCTGCAGCAGTACTGTTAAATGAGTGTCTAGTACTTGACTTACCATTTTAATTTCACATAATTC
TGAATCTATTTTTCGGCTTGAATTCAAATAACCCACTTCTAACTACCGAAAATAAAGTAGAAAAATGAACTCACTACCAGACTTCAAAGCTTAT
CCAAAGCAAAGATTTCGTCTTACAGCCAGAGGTGCATGATTGCAGAGCTGGATGTAACCTGAGAGGCTAACTACTCCTAATTCAGCCTCCTTGA
ATGACTGGATGATGCAACCTATCACAGGTGTTTTAGGTTTTTTCATCTCTATTTAATGTCCCACCTCATGCTTGAAAATCTCAAAGTGAAAAAG
CTCCAGTCTGAGTCTTTGGCCAAGCAGACTAACTGAAATGACCTGTTAGCCTTTGTTGTATCCATCTGACAAGATCAGCTTGTAGAATCCAGAA
TGGAGGTGGGAGATCAGCTGGCAGCCTCATTTCCCTCCTCAACCACCCCTACGCTGATGTGCCCAACTCCACTCACAGCTGGGCAGCCACCGCT
TGCTTGAAAATAAATCAATCTACACCACTCTCATACGGATGCTCTCAAGCATCCCTTGTTGTGAGAAAAACATTTATTCCTGCTACTAAAATGC
GTAAAGTTATCTCACAAACACATACTTACAACAATGAGGAATGAGAAACAGAACCATCCATAAAACTATAAGAAACGGCCTGGCCAGGGCAGAC
CAGGAATTAGTACTCTCTCCTTGCCAGGGGTCAGGCAGGGACCTCACAAGCAGGGTGTTTTGTCCTAGGGGGCACCAGACCAGAGGTCAGGACA
CTGAGGGCTACTCCTAGCTCTAGGACTTTTCCTTATCTGAAGGCCGGGTCGGTTAGCAGAGAAAATCTCACAGTCCCGTGCCAGACCTCCACGC
AGGCACTCTGCAGTGCTGTTAGAAAAATTAGCTCAGCACCTTCAGCTTGCTTTCAGCCATCACTTAGCTTTCCACACAGACATTCCAAATGGGG
CCTTTCAAAACTCTGCCCTCAAATGAAGGTAGTTCAGAGCAACACTTTAGATCATATCACAAGACTCCCATGACGCTTCACTGCTTCCTCAGCA
TACGCCAGTGCCCGTTTTACAGGGGGGATTTATGATCCTTCTCTCAGTAGAGCGAGCCAAACTTTTATAATATTCATTAAAAATCAGCACCACG
TGTAATAATATGGAATACTGCCTGTACACAACAGGGCCAAGTTATAGGTGflTAGGGAAACGTGGCAGAATTTTCTGGCCTTGGCAAGAGGTAA
AGTTGCAGGCACCAGCACAACACTAACACAACCTTATTTTATTGCAAATGTACATTCAGCCTAAATCCTAATTACCTTCTGTAAAATCCTAACC
ACCACCAAGCTTTCTTCTGACAGGGCACTCACACGACGGCCTTTAAGGGCAGCAACAACATCCTAAGAGCCGCAGAAAGGAAAGGCAGGGGAGA
GTGGCTGGAAGGCAGTAGGAGCTGGGCCTCAAGCCCGGGACGTTGGCTTTTAATGTCCTCCTGAAATTGACCACTCTCTTTCCAAAAACACTGG
CTTGGGGGCTTAAAGGTTCGCTCAGTAGGGCAGGCGGAGGCCCTCCTGAAAAGGATTGAGAAGTCAACATTCAAAAGGCTGCTTTTACTCGCCC
GGTCCCCACCCAAAAGGGACTCCGGTCACCTTTGTGGGGGCCAGTGGAGCATCTTTTCTTGCCCTTCCGCGAAAACGCTAGCTAACCCTGGGAT
CCCCGGTCCCGACGTCTGCTCTCCTTCCCCAGGTGCAGCACCTAGGGCTCTGGCCCCCTCCCCACGTGCCACACCAGCCCTCACCACTACTCTC
ACCCTGCTATGCCCCCAACCCTCGTCTTTCGTGGCCGGGAAGGCCAGGCCGAGCGGGGCGGTCAGCCGGGCCGGGAGGAGGGGGCTCTCCGGGC
CGCGCGGCCGCACAAAGCGGGGAGATCTGCTGCTGCGGCGGCATCGAGGCGCCCCCCCTCCCGCGAGCCAGCGGAGCGCGCGCGCCCGCCCCCG
TCCCCCGCCCCCCCCCCCAGCCCCCCCACCGCCATTCGCGGAGCGGCTTACGCTAGTCGCCGGGCCTACCGCCCCCCAGCGGCCGGGGAGGTGC
GCTGCCCGGCTCCCGTAAAGTTATTGTGAATGGGGAGCGGGTGACGTCAGCGCCGAATCTCAACAATGTAGCGATTGAGAGTGTGGGCGTTCCG
GGGAGACCGCAACCCCCGCCGCCCGCAGCAAACAGCGCCGAGCCGCCGCCGCCTCAGCAGCACCAGCACCAGCAGCAGCGGCAGCAGCGGCCGT
GCACGCCCGGGCTGCGGTCGCACAGCGCTAACGTGAGCGGCCGCCGCCCTCGCCACCCCGCCTGCCCACTCCCGCCGCCGCCCCGCTCTCGCTT
TCCCCCCGGCCTCCCCTCGCCCCTTCCCCTCCCCCTTCCCGGCGCACTCGGGGGGCTGGGAACGAGCTGCCATGTGATGCGCGTCCCCTCCGCG
AGCTTTCGGTGACCCACGAACTGCCCACCTCGCCGCCTGCCGGCAGCGGCCTCCGAGCCGGGAAGACGCGGGGAAGAGGAGGCGCAAAACGACG
CAAAGTTCTCCGGCGAGGTGAGTCCCGGGCCCCGGGCCCCCGGCCCCGCCGCTGGGCGGGGTGGCGGGGCAAGTTTGCGGGGGGAGCGGGCGCG
CGGGGCGGGCGGGGAGGCGTGGGGAAACGGCGCGGGAAGGGAGCCCCGGCCGCCGGGCGCGCAGCCGCCCTCCCCGCGCCCTGCCCCCTTTTAT
GGCATTTTCTAGCAACGGGAAGGGGCTGCGAGCCCAGCCGCCGCCCGCGCCGCGCCGAATGTGTGCTCTCCCTCCCGGCTGTTTACGGCGGCCG
AGGCGCATTGTATTTTGCTGGCGTCGAAGGGTGTTTTGGAACAGCAGAGGGAGGAGGACCACAGAGTAATAACTCAGGCATCGCGAGGGCGGGC
TGCAGGAGGCGCGCGTCCGCGCGAGCCGCAGCAGCTGTCAGACCCGCGGCCCTTCCCCCGGCCCGGCGAGGCCCGGGTGTCCCGGCCCCCAGCC
TGCCATCCCAGCTCCAGGCCGGGCGGCCTCCTCCTTAACCCTCGGCGTGCGCATCCCGGGCTGCTCCGCCAGGACCTTCGCCGCGGCGCCTTTG
CGCTCAGCTGTCAGCCGCCCCCGGGTGGCCCGGACCACCGCGACCGGCGGGGAATCAAAGCCCAACTTTAGGCTCGCAGGAAGAGGCGAGCTGG
CGGCCTCGCCGCCCAGACCTGGATCGAATCTCTCCTAGCGAAAGATCGGGTTGTCCGAAAGCGTCCCCCGTAGCTAAGACCTAAGGGCGCCAGG
CTTCTGAGAGGGAAACCTTGCACCTCCTCTCCCACAGATCCTCCATGAAGTCAAGCTGCAACACACACACACACACACACACACACACACCCTA
CACTCCAGTCTCCAACCCCCACAAGCTTCACCCAGGAGACTGGTTTATGAAGGGGACACTGCCCTCCCCGTCCCCGCGTCGAGGCGCTCCGTGG
CCCCGCTGTGCTTCGCTCACCCGGCGGGTGTTCGGGAGCCCGGCGCCGCTGGAGACGGCGGCGGGCGGCGCTCACGGGCTACCCTAGGTGCCCA
CGAGGGGAGAGACTGGAGCGAGCATCTCGGCCACCTCGGTCTGCCCAAGCATTTGCAGGAACTCGGACCGTGAGGGGAAGCCTGCAGCGCCGGG
AGTGAGTGTGTGAAGCGGTGGCACACTCCTGCCTTCTGGCGTCCCGGGCGCATGGGGCATCCCCAGCACTCTCCCGCGCGTAGGCGAGTTTGGT
AACTTGAGGGAAGTTCCGCCGCGGCCGCTGCCGGGGTGGTTTGTCTGTGGCCCGATGAGGCCCCCCTCGCACCCTGCTCTCCCGATCACCGCCG
TCGCCGTGACCTGTGGCCTCCAGGAAACGAGAGGCACCGGGGCGCCCCACCCGCGAGACGGACCCGCAGTCTCCCGGCGGGCCCCCGGGCGGCG
AGACGCGGCGTTCCTGGTTTTCACCTTCGCTGTCTAGTGCTGCAGTCTCTCACTCTCTGTAAATTTCATCTGTTGAGTGTATTTAACTAAAAAG
GGTAATTACTTTAGATAAAAATCCTTTCCTGCCTAGTGGGAACCTTGGCGGAGATGATGCCTAATGAGATATTTTGCTTTTGTCTGTTTAGAGT
AAAATTAGAGATGTTAACGCTGATAACGGTTTTCTCAGATCATTAAAATGTAACATGTAGGAAAGGCACGTCTTGCTGCAAAGGCTCGGGCTGA
CATGAAAGAAGCTGAGCGTGAAAGGCTGCCCCTCCTTTGGCGGAGAGGGCTATTTTGCTAAGGCAGCGGGCGTCTTGCTTAAAATGTGAGATCG
CCCCACCCTGGTTCTCGGGCACCTCCCGAGTAAACGGCGTTTTTCATTCTTGCACGTAATAATAAACGTGAGTCAAAATGACATCTTGTTAGAA
TTAGTCTTTAGTCTGTAAGAGAGATTGCCAAATTACAGTGTTGCGTTCAAACTGCTCCACTGAAAGGTCTGTCAGCATTTCCATTATAAATTCA
GTTTTTCTGGAGTTTATAATTTCACCACAAAACTTTGAGTGGCTGAAATGTTTTCCCTTCTAGTATTCTGGTTTTTGAAAGAAATTTATTGATT
GCCCATTTCCAACGTTTTTTTTACTCTCGAAACCTGAATATCATTGAAATAGAGCGAAGTCCCACAAATGGAACACACAACCATGGAATAAAGT
ATTTTTGTTAGAACATAACTTTATTGGTGCAGAGCCGTATTTTCGTTTTGCATTGCAGGAGGCTTCAGGTTTGCCATTTGGTAATCATGAATGT
GTTGAGTAAATTCTTCTGCTGTCTGTCTATTCTGCAGGTGGACTGAAATGTCAGTTGGTAGTGTGTTTGCCAGGATTACATGGAGACACACACT
TCTCACTTAAGTCTGTGTCTGAACTCATAGCTAAATTGTGAACACTCCTGTTAGTTACTTTTTTGGTTCTTTGTATGTTGCATCTCAGGATCTG
CTATGAAGAAGAAGGGGTGAGGTGAAAGAGAGGGAGGGGATCAGGATTCACTTTCTGTATTACCAGTGCCTGGGAGAAAGCCATTTATTATTTT
CCTATTGAATTATGCCCTTTGACTTAGTATCTTCAGGCAATTCTTTTCCAGTAGCTCACCTCTGGCTGCTGTGTAAGGGGCTTAATTATTTGCA
AACTGCCTTGAAATCCCTGGATGAAAGGCACTGAATAAGTGCACACTATTGTTATTCAGATATGGAATTTAATGATCCATTATAGTTCATCAGG
AAAATGCACTTTGCAGTGATGGTAGGAAATGTTTCCACCTGATAGAGGGTAATAATAACTCCTTAAAAGTAGATTCAGATTACAGATCAGACAA
TTAACTCTAGCCATGTGCTCTTCAATTTGTTCCAATTCATTTTGTGATCAAAGTGAGTCACAGAGGGCCAGATGGTCAGGTTCATGTTCAGATC
TCTCCTAGGCCAGCCCTTCACTTCTCGCCCCACTGTCTGCACAGCCGACAGGACTTAGTTATTCATTACCGCTTGTGAGCTGTTTCTCAGAATT
CCTAATCGAGGCTCCTGTGATGCCAACAAAAGGGCTCAAGCCAATCTGTATTGTTGAGAGCAGTTTAATTTTACTTGGATTAACTTTCAGTGTC
TCTGGTGTGGATCCAGTCTTAACAAAAAGGAAAGAGACCCTACTGGCTCTCTATCTCTGCTGTGCCTGGCCCCTCTACTCTAGTTTTTGAACCT
CAGATTTCAGGGGTGTGACATCTAGCTTATTGTAAATGCAGGCTTGATTGTTAATGAATGAGAGAATACTTAGATAAATAAATATCTTCCTGTG
TGTCCAGGGGGTGAGAGCCAGTACTAGCTTCATGGGCATAAGACCTGTGCAGTTACATAGGCCCCCTTCCTCTTCCCTGCTCAGAAGGACCTCA
TATTTGGTATAAGGTTCTGCTGTTTTCTTCGGAAATTCTTAATAATTTTTGAACAAAGGGCCCCTCGTTTTCATTTTTCACTGAGCCCTGCAAA
TTATGTACTTACCTGGTTCTGGTGAGGAGCAGGGCATAGTGTGGAGTGGCAGGCCGCCCCTACTCAACTCATTGTAGACTCTTGCTAGGGAACA
GTTGGTAGGACTACTGGCTTTGTGTTGCCACATCATTTGATTTTTCCAGGGGAAGCTGAATATTGGATATTTATCTGCAATTTCCTGATTTTTA
AATATTGGCTCAAATCTTTAAAAAATTTTTGGATGAAAGCATATGTAACAGCTACTGTCGGCCTGGGGGCTGCTTGTCTCCCACTACTGAACAG
ACTGAAACATCTTTTTTTGTTTAATATCTTTGTGTGTCTCTGTTTTCTAAAGGCCTGGCATATAGTGGATGATTTCTCAGTATGCACCCAATGA
ATGGTTACAACTACCTCCAAAATACAAAGATGCCAAAAAAACAAAACTTCAAATCAACATTGACTCCCACTCAGGGATTGATCACCTTCCTTTT
TAACAGAGAATTCGACATGAAAGGCCGAATAATTTAATACCATGAGTACAGTGTGATTCCTTGCAGGTGAATAATCCATTACTTTTGAACCACC
TGCCCTCTATTTAGTTGTTACTGACCACATCGGGTAACTTGGCTGGGTAATCCCTGACGACCTCCATGGGAGTTTGACATTTGAGAACACTCAC
CAAAATAATAGTTTTGATTTACTGAATCCGCACCATGTGCCACACCCAGTGCTGACTACCTTGCCTGAATGTACTTCATTCATTCCTCAAAGCA
ACCCTTTGAAGTAGGTGTCGTGCCTGCCCATTCTGCAGAGTTGGAAGAACTAATCTCATGTTGCAATGAACTTAATCTGAATATAGTGCATGTT
TTCTTGAATGTCTGAAGCTTATTTCCCCATTCCATTACACAGTCCAGTCCTCAATCAATGACTGGGCATGCTGTAGGACTGTGACATACACAGA
ACATGGTCCCTGCCCTCACATAGAACAGAGAACAATATAAAATGTAACATATTTGTCAAAATCTGCAATTCAGATAGCAGACGCTGTAAATCTG
CCTCCCTGTTTTTCTCCTTTCCCTGTCTGGATGTTGAAGAAGTTTCAGCGACATATGACCCTGTCAGAGGTACTCACAGTTCACAAGTCAATGC
TGGTTGCAAAAATTGGAATTTCTTTGGAAAACAACACAAGCTTTTGGCAGAGCTGGGCCCACATATGGGAACGCAGCACCAAGGCCTTGCGAAT
GACGGAAAATCGCTTCTGTATTGTTAATAGCAATATATGACCTCTGCTGTCCTCCTATCTTTAAATTTTATGTAAATATTTACAAACTGTTCAT
GTCCAAACTGAAACCTGCTATGAACTAAACATTTTGTAAACTCAGTAACATTGCCCAAAATACAACTCAACTTTCACCTTTTTTTTTTTGTTTG
TGATGACATTTAATTTAAAAAAAAAAGACAGTAGTTGCTTTTTTTTTTCTTTTTTTTTGTCAGTTCAGGGTCTTTTAATTTTTTCCCTTTACTT
TTTTTTTTGTTAATTTCAAATGTCAGTCTATCTAAAACTGAAACTTAATTTTACTATCAGCATGCTACTCAGCTTCCGTAACGCTGGAAATGTC
CTGCGAATCTTACCACTATGTCTTTTCCAAGGGCCATTTCATTTCTAAAGAATGTATAAATTGAACCTCATCCTGCTAGTGACACTACTTCAAA
ATTCAGGCAGGAATTTTGCCATTTGTTTTTCCTTGAGCTATTTTTTTTCCAAAATAATAAATCTTCCCAATCCAAAAGCATTTCACATCATCCT
TCATTCTAGTCATTGGAACTACAGCCAAAATGCCTCTCAGTCTTCCCCAGTGACTTCATAATCACCGTTTCTTCAGCATATTCCCTGGTTGACT
TCTCCTTTGTTCCTTAGCCTCATAATGAATTCCCCTTTGGTTCTGAGATGTGTCATTTTCCTCCCTGACCTTTTTGTTCTTTTCCTGAACGCTG
TAGGACATGCTGCTGCTGATGTGATGAGAGCCGGCGCTTGGTTGCTAGGTCCCTCCTGTCCTTGTGTCAGCTTTCATTGGCTCCTTTCCTAAGC
ATTTAAATCCTCCACTTTCCAGTCCTCCCATTCATCTCATTATTCTCTTCATGTGTGCCTGAGCCTGTAGCTGGCATGTAGACAAAGTCCCAGT
TAGTTAGAAGAAAGCTTTTCGAGTTTCCAAGTCTTCCATCACTCTAGTCTTCCGCCATCTTCTATTCTCCCTCCCCTGTCACTTTCTGCAGATA
CTTCAGGAATTTCCTCAAGACTTTCCAGCAGTCTAACCCAAAGACAGCTTAGACCTTCACTTCGGATTTTAAAGTTGGGAGATCCCAATGTGAT
GATTTCCATTCCACAACTAGGAAACAAACTGAATCTACATGCTCAGCATCTTTCCTTAGTATTTCAGTATCCATAGATTTTTGGCAATTTTTCT
TTTTTTCCTATCCTGTATTCTTCTAAGCAACAGAAGCTCGTGGATAGTGCAAAATGGCCCAAGTGTCCAGAAATACTGTTGCTTAGAGGCAGAT
GCCTCATTCATTCATTAAACAAATATATACTGAGTATCTCTTACATGGCAGCCATAGTGTTAAGTGACCACAGTGCTAATGGGGAGCAAACCCA
GTCCTGGCCCCTGGTCTCATGAAGCACGTCATCTGGTAGGAAGAATGGCACTGATCAGTTAGTAACAGATAACCCACTGCTCGTGAAGGAAACT
ATTTCATACACCAGAGTGTCTAATGGGCAGATTTAACCTGGTCGGGAGGTTGGGGAAGCCCTCCCTGAGGAATTGTGGAGTGACCTTCCATCAG
GGACACTCTCAAACTTGACATTCACTTTTTCTCCAGCTTCCTAATCCTATTAATTCACTACTATCTTTCGCCCTTTGGAGTATTGCCTAAGCCA
TGCTGGAACCTCTGGGGCCATTTTGGCAAATAATAGTTCCTGTATACAGAGTCCAGAATTGATTTATTTACCTGGGTCTTTAGAACCTGATAAA
AACAGAAGTTAACTGCCAAGAGTACTGTTCATTTCCATACTTGGAAAAAATACAAGTTAACATTCTTATTTTGAACATATTTAGGTTGGTTTTG
ACAATATTTGAATGATCACAAGCTACAAAGATATTCTCTCCCAGTCAGCTGATGTGTGTAGGGCCTTTGCTGAAAAAGTGACTCTAAATAGTCT
GTCTGAAGTCCCTGGCTACTCCAGAGCAGAGGGTCTCCATCCTCTGTGGCCCGAAGATTATTCCAAGCTTGTCCAACCCATCGCCACCAGGCCT
CTTGCCTGTTGAATGCGGACCAACACAAAGGTTTTTTGTTTTTGTTTTTGTTTGTTTGTTTTTTCTTTTTTCCTATTTTTCTTGTTTCAGCTCA
TCATGTATCATTAGTGTTGAGTGTATTTTATGTGTGGCCCAAGACAGTTCTCCTTCTTCCAGTGTGTCCCAGCGAAGCCAAAAGATTGGACACG
TCTTATTTAGACCATTCTTAGATGCAAAATTAAGGAATTTTGACAGCATCTATTTAAAATAAATTGATTATACTTCATGCTAACACCCCTCTAT
TTTCTTTTTCTTTGATGATAGTTAACTTCTATTGAGAGCTTCCTCTCTGTGCTGCTCATATTAACTCACATAATCCTCAGAACAACCCAGGGCT
AGCTGTGATCACTAGAAGCCGCAGCATAGGTGTCGCTCAGCTAATGAATGGTAGAACTGGAGTTCGACACTAGCTGTCTGATGCTTGGGCCTGT
TCTCTTACCATGTTATGGCTCTCCCTTTTCCTTTTCTTCTTCATTTGAAGTTGGAACTCTCTTTAGCAGTTCCCAGAAGTAGAAATAAAGGTCA
TCTCACTCTAAAATATTGCATGTTTTGTTGACATCTATCTTAAAGTTAAAAAATAATTTCTCAGTAAATCTGAGTGAGTATATCTCCTGTCTTT
CACATGGCCTGCTTTCTAATAATTGCATTCCTTAGGTTTTAGTGTCCATTTAGGGATCGGAAAGTAAAAGTGATGCATATAAAGGCAAAAGAAG
TACTACGCATATAACTGAAATACCATGGCTCTGTCTCCAGGGTTTACTTGTAACTCACACTCCTACCCTCTTCTTAGGTGCAGGGAATTTGTTT
TTTCCCTTTTGTTTTTTGTTTTTTGTTTTTTTGGTGGGTAGGAAAAGGTAAAAAACTATACCTGTAAGAGGTAGAGTATTCTACCAAGGAGGAA
GATGTTCATTCAGAACTAATTCCATCAGAAATCCTAATTAGAAATATTAGCTACCAGTAACAGATAAAAGGAGGTAACCTTTGTTCTCATTCTG
GTCAATTTCAGGAAGAGATTATCTTGCCAGATTAGCATGTTGCAACTCTCAGCTGGAGATTTGAATGGTTTTAAAGGACTCAGCCACTTGGTAT
GGCAGTAACCCCCAATTACCTCAGGTTTGCAGGGTCACTGTTTAGAATGCTGACTCAGCTCTGCCTGGGCCAGACACCGAAGTTGTCATCTTTA
GTCAAGAAGCATCATTCTCAGGAGCCACTTGAAACATTAAGCACACATTTGAAGCTCAATTTGGGAAATGATCAAGTGTATGAATGTATAGTAC
TGTGCTTATTTTTCAGCATTTAGTTCAAATTTTGGACAAAGCTAAATGGGAGATCCTTACTGTGTCAGTGTTGGAGGTTTTAAGTGATTTAAAA
AATCAAGCAGATGAGTGCATAAACATTTCTATGCAAACAATCTTCAGCTTAAATTAAAGCTGCTGATTTAGTGTGAGAATTACTTGCATTGATT
TGTAGAATATCTATGTGGGTTCAAGATTCCATATTATGCAAATGAATAAGCCCTGAAGAGTCAAACTGAAAAAAAAATGAAATTAACTGTGAAA
GATATTATTACCCATTCAGCAAGAGGAAAATGTAGACAGAGTTAACCCTTCCTTTTCATACATTTTCTTGGGAAGATAAGTGCCAGGGATGATG
GTCCGTGTCCCAGATGTAAAGAAGCTAGGCACGGTTTGCAGCCAGTTCCCCCATCCTCCTTCTGTGCTCACTTTTTTGTTTCATGCTCTCTTAC
CTTTTGGCCACCCAGTCCCCTCAACTAGTCCTTGTCCTTCCCTTCCAAAACCAATCTCAGAGAAAGATTTTATCAACACAGTTTTACTAATATA
TAAGTAACTGAGCATACTTAATGTGTTACTTTTCTGAAGCATCAGCATGTTAATAGGAGACAAAGCCAAAGAAGTAAAACCCTAAAGATAAAAA
CAATAGAATTTCGCACACTGGTAACCTGGTGATAGGAGACCATTTCTGAATGGAGAGAGGGCTCGTGATAAATAAACACAACCCTAGGCAGAGA
ATATAGGCAAATGCATTACAGTATTTAAATCAAAGTAGTATTAAAATACAAAGAGAAGATTATTTTTAATAATACATTTGTCAGTGAGCATTTC
CCGCCTTTTGCCGTACATTGGAGTTGATCACATAAACCTCTATAGCAAGATGGTATGTATTTAACATTTAAACAAAGCTTCCACGGAAAAGCCA
TTTGGTTTGTCGTTAGCGATTTTTGTTATTCAGTAGCAATGAACCTGAAATTCTATATTATCTTTGCATGTTGAATTTTAGAAGAAAAAAAAAG
CAGCGGATCCAACTTCACTTACCTCCCCACCCCAGTTGCATTGATACAAATTGAATTGATAAATTTATTTCAAAATTGGTCTGTCCCAATTTGA
CATACCACAAGCATAGTGGTGTCAGCATGGTGCTGACATCTGGACATTAGGAGAGTTGGTTGATTAATTAAACTATACTTATTTTAGATAGAGT
TGTTGGCAGGTAAATACCTGGCTGTAGAGTCAACGCCCTTCAGTCCCACCTCTCCCCCACCATACCCTTCTCCAGCAACCATGCTATGTTGAGT
TCATCGCTCCATCTAGTGCCAGTCTGTGGAACTGTCTTGCAGGACTTCACTGTCACCTCCAGAGCTGGGTATGATGAAGAGTCCTCTTTCTCTG
GGAACACACTGTCTAGCGGGGAACTGCCCTCTAGTTCCTACCTCTCCTTTGTTTTTGCAGTTTAGTTATACCTCCGTGTCTTTCTTTGTTCCAG
ACTTAATTTGTTTCCCTCTCTCTAGTACCCCCTCTTTGTACTTGTTTCTCCTCCTTTTTTGTTTTCCAACTCTCAATTTTTTTTTTGGAGAACA
AAACAGTACTTAAAACCGGTAAGTATAAAGATTTTTTTTCTAACAATACTTTATGCCAAAGTAAGAGAATTCTATGCATTTTAAATGAAACTAT
CAATTGAAACCATGTTAGAGAACCAGAATGACCCTAAAGTTGAAGTCTGAAAGTGTCACTTGGAAGACTGTGTCCCTTCATCCTCCTGTAGTTA
GACATTTCCTCAAACACAGTTAATCAAGAGTATTTTCTTTTAAATGGTTTTGGAACTATCATTGTAGTATCATTTTCTTTTTAGATTATAAATT
TAACCTCTCATATTTTAAGTTCAAGACATTTTCACGGACTATCAAGGAATCCTAGCTCATTGGCCTGGCCCTATTTCTGTGGGAAAATGTATTC
TCACTTCCACACAACCAGTCTTCCTGCAGATTTTTAGGTCCCGTATAAAACGAGAGAACATAGATGAATGAGAGGCATTGTCAGACAAAGCCTC
GCACATGAGATCTCAATTAGCATACAGGTTTTAAATTTCCCACATTAAACAGTACATTTTAAATTTCCACTTATGTTAAGCGTGTTGATATTGT
GGGTTTGAAAGTAAGTCCCCTCTGAAAAACAGGAACTGTGCTTGTCTGTTACCAGTTCTTGTGGTCCATATGCTAACTATTTTGATTTTTCAGA
AGGGAGGAAAAGCAGTTAAATCTAGTTAAAATTAAAACCAGTTAAAATTAACTCGCTCGTGACATTTCAGTGCAGCTGTTCCACACATAGCAAC
ACTAAGAGTGAGTTGTTTATTACCATGATTAATTATTATTAGTTGTTACTTCTTTTTGAGCAGTGGCATAGATTATTTATATTTTTGGTCTAAC
CTTGGAAAATAGAAACTGCAAGCGTAGAGAGGTTCCCGTTTCCTAATTTGCATGTACCCTGTTTTTGTTTTTGCCAGGTAGGTGTGTGCATCTC
AAGTACACGAGAGTAATTTAGCATGTCAGCACTTTCCTACCAGCAGTGCTTGCTGACTCATGCAGCTGTTGTAGCACTCAGCTGGCCGCACTGT
AAACTGCCCATAGTACCAGCACCGCCCTTGGAGAGCTCCGGGCTGCTGGGCTGGTTAGCTACTGGGAGGCTATTTTCAGATCTGTGGTCACTGC
TGAGCTGGGTCCTCTTCTCCTGCCAGCAGCCAGACACAGCCTTCTGGTTGCACCGGAGAAAATTGGACAAATTTCTACCTTTGTAGGGACTCCA
TAAAGATTCATAGCATTTCTGGATATCCCTCAGGAAAACTTTTTTTTTGCTTCACTTTATTTTTCTGGGAATATTTAACGGGAGACGCAAAAGG
GGCTGATAATAAATGAAAACGGAATCTGCTGTTATTTCCTACTAAGATCAGAAAGAGTTTTCATAAACCTTTAACTTACTTACGCTTTCTTGGC
GTAACTGAAACCTGACCCACTTCTCCCTCAGTGCAAAGCAGTGTGTCCTGAGTAGGGAAGCAGGGGCGAGGGTGCAAGTGACTAGCCTGACATT
GTTTTATGTGGCTGAAGTCCCTAAGTGATGACAGCTTTTGTTTATATCCGTTCCCTGTTATTGAGGTTAAAAAACAAAACAAAAAACTTGACCT
CAGGTCATTTTAAAGCTAGCATGTATTTTCAGAGATGGTTTCACTGTTTGTTTTCAGAGTTCAGTTTTCCTTATTTTCATCCCAAAGTGTGAAA
TGTTGCTTTTCTGAGCCTTCTAAATAAACTTCCTTTTGTAGATACAAGTTGATCCAATCATCATAAACTGTCTTGAAAAAGCATGATTAAAGTA
AGATCAGCTGGGGAAGGGAAAGCTTTGCACACCCTTGAGGAGTTTGCCTTCTTTTTCTTAACTTTCAATAAGCATTCTGAAGCAGCTTTGACCA
GTAATAAAGAAAAGCCACAGTGACTGTGTGTAACATGATGTTGATGGAAGTTCTTGGTTTCCGCCGATACGAATCACTCATCAGTATCAATAGA
ATTGTGACTTCTAATAATAACACAGTGCAAGACCTTCAAAACATGTGGTTTTAGCCCTTGCTCTTAGTGAAAGGTTGGAAAATGTTAGTTTGAG
CTTTTTTTTTTTAATCTCTTGATTTTGCCCAACTATAAAGTTGTTTTTTGTAGGAATGATGATCCCCCTGTTGTTTTCTCATCTTACTGTTTTT
AGTTTCATTATGATTAAGGAGCCAAACTCAGATCATTTCATCGTTTAGAAATCTCTGCGCTAAGCATCTCCTGTCGGGGCTGTGTTTGAGGGAA
ATGTGGCTGAAAAGCAAATCCTCCCTGAATGCAGAAAGGAAAATACTGGTGCAGACTACAATCCACTGAGCTGCGTCCCTCCTACACAGCATCT
ATTGCGGAGGGCTGTGGTATTGCTAAGTAGCCCCATAACCCTGTGAGTCATTGTCAAAGGATTCCAAGAGGAAGGTTAACAGAAAATGCGAGTC
AGAGGAAACAGCAGAGTGGAGCAGCTAGCTCAGGAAGAACTGTGCTACCTGCTGACGGCCTGCCCAGATGTCACCAGGAATTCAGGTGTACGTT
CATCTTTTTGCAGCCCATTTCCGTATGTCTACATATGGGGACATTTTTGTTTTTTCTTTAGTTTTGGTTGGTTTTCCTGCACTTTGGCCAGACT
ATCAGAGATCACTATCTTAGCTTCGTATCACAGTGGTCTTATCCCACAGCCTTTGCTTTTTGTATTTTATAGTCAAAGCAACTGAATTTCTAGA
CTGTAAGACCAACATGTATATTTAACATAATTCTTTTCCTTTCTAAGTGCTTTATTTATTTATTTATTTATTTATTTATTTTGAGATGGAGTCT
TGTCCTGTCACCCAACCTGGAGTGCAGTGGTGCGATCTCGGCCCACTGCACCCTCCACCTCCCAGGTTCAAGTGATCCTCCTGGCTCAGCCTCC
TGAGTAGCTGGACTACAGGCACATGCCACCATGCCCAGCTAATTTTTGTAATCTTAGTACAGATGCGGTTTCACCATGTTTGCCAGGCTGGTCT
CGAACTCCTGACCTCAAGTGATCTGCCTGCCTCAGCCTCCCTAAGTGCTGGGATTATAGGTGTAAGCCGCCGTGCCTGGCCAGACCTAAAAAAT
TCTTATAAAATAGTTCAAAAGTTAAAAATGCATGAGGAGGCCGGGTGTGGTGGCTCACGCCTGTAATCCCAGCACTTTGGGAGGTCGAGGCGGG
TGGATCACGAGGTCAGGAGATCGGGACCATCCTGGCTAAAACGATGAAACCCTGTCTCTAGTAAAAATACAAAAAATTAGCCGGGCATGGTGGC
AGGCACCTGTAGTCCCAGCTGCTCGGGAGGCTGAGGCAGGAGAATGGTGTGAACCCAGGAAACAGAGCTTGCAGTCAGCCAAGATCGCACCACA
GCACTCCAGCCTGGGCAACAGAGCGAAACTCCATCTCAAAAAAGAAAAAAAAAAAAGCATGAGGGGTCTTTCACTTATTGTTGGAAAAAAAAGT
GAGTAGGACTTCTGTTCACTGAGTGGTAACTATGGAGAATGAGAAAACAAGCCCAAAGTGGTAAAGACGTTTGTGGCACATAAATAATACTGGT
TCAGCACACATGTGAATCAATAAAAAAGCCAGATAACCAAAAAGAAAAATGATACTGTACATTCATATTCTTAAACATCCATTTCACAAAGGAA
GAAACCCAATTGGCTAGTAAATATATAAAAAGATGCTCAGCTTCATTAATAAACAAACACATGCAGATTAAAATCATACTAACATATGATTACA
CACTCTCTGGATTGGCAGAAATTTTAAAACCTGACAATACCATGTAAGGATGTGAATGAATGGGAGTTTTTGTATTTAGTTGACAGGAGTCTAA
CTTGGTACAATTATATCGGAAAACAATTTGCTGTTACCTAACCAAGTTCAACCTGCAAATAACTTACAACACAGCCATCTTACCCCTAGGTATA
CACCTAAGAAAAATGATTACCTATGTGCTTTAAGAAACATGAACAAAACAAAAAATAATTGATGCTGGCAAGGTTATGGAGAAAAAGGAACACT
TACACACTCTCGGTGGGAGTGTAAATCAGTTCAGCCATTGTGGAAGACTGTGATGATTGCTCAAAGTCCTAAAGATAGAAATACCATTCAACCC
AGCAGTTTCATTACTGTGCATATACCCAAAGGAATATAAATTATTCTAAGACACATACACGTGTATGTTCGTTGCGGCACTATTCACAATAAGC
AAAGACATGTAATCAACCTAAATGCCCATCAGTGATAGACTGGATAAAGAAAATGTGGTACATACACCATGGAATACTATGCAGCCATAAAAAA
GAATGAGATCATGTCCTTTGCACGGACATAGATGCAGCTGCGGCCCATTATCCTTAAGCGGACTAACACAGGAACAGAAAACCAAACTGCACAT
TCTCACTTATAAGTGGGAGCTAAATCATCAAAACGCATGGACATATAGAGGGGAACAACGGACACTGAGACCTATTAGAGGGTGGGAAGAGGCA
GAGAATCAGGGAAAATAACTAATGGATACCACGCCTAATACCTCAGTGATGAAATAATCTGTATAGCATACCTCCATGACACAAGTTTACCTCT
GTAACAAACCAGTACATGTGCATACCCCTGAACTTAAAAGAAAAAACATGGACAAACATATTCAAAGAAGCATTGTTCATAAGAGCAAAAAACT
GGAAGCAACCCAAATAAATCCCCATCAACATTCAATCGAATCAGCACATTTTGCTGTGAGGTATTTATACACTGTCATACTATTTGGCAATGAA
AAATCAACTAGTATAACCACATAThGAAGGGTGTGGAGAGGCAGCATTTCTTGGTGGTCAAGAGGAAGGATTGTGCCTGGGTTCAGATTTTGGT
TCTGCCACTTATTGTGTGACCTTGGATGAATTTCTGAACTTCAGTGTACCTTAGTTTTCTCATCTGTACATCAGGGAAAATGATGTCTACCTGA
TAGTGTTGTTAGGAAGTTTAAATGAGTCAATGTTTGTGACATGCTTAGGAGAATGACTGGCAGACAATACATTCTATATGTGTTTGTTAAACAA
ATAAAAGTGCATCAAAATGCATGAATCTTACAAACAATATGGAAAGAAAGAACCAAGATAGAAGAATATATACAGTGTGATTCTATTCATCTAA
AGTTCAAAAGCATGCAAAACTAAATATGTTATTTAGGCATACATACATAGATGGTAAAACTATAAACAAAATTAATATGATTTAATATGATTGT
CACAAAAAGCAGTATAACAGCTACCTCCACACGTCAACGAGTGGGTATGATAGAGAACGGGCTCACTGGGGGCTTCTGAGGTCCTCGCACTGTT
TTATCTCCCAACCTGCACAGTGGTCCCAAGGATATTCTTTCTTCTTAATTGTTTTTAAAAACTGTCTATGTTTTTTATATATGTGTGACCTATT
AGTAAACATTTTAAAGGATTTATGTGAGTTGTTATAGTAGCAACATGACATTAAATAGAAATAACTGTAAATTGCTACATTTTAGTTAAAAATA
ATTGTTCAAGTGCACATGGGAACACTTGCTTGGTATTATTTCCTCCACAACATTGTGAGCCACCAGTGCAGTGTGATTCTTACAGAAGGCGACA
CCTCCTTAAGGCCACATTAAGAGAGCTGTCCCCTTTAGATCAGGGAATGTAATGTTCTCACTCAACTACTCTCAGGTCTGGATGTCCCATGTGT
GGGATGCTATGTCTTGTCCCAAGGGATTTATTTTAAGAAAGATTTTCACCACAGAGGGAGCATTTTCTCTCATAAAGATGTTTAGGGGTCTGGA
AACCTATCAGTTCTAAGAGTGGCCAAAGGAAGTGGGGATTTTCAACATGGAGAAGAGGAGATGTTGGGAGAGAAAGAGTGTCTTCCATCACTAG
AAGTGGAAGCAGCCTTTCCTTGTTGCTTCCCTCTCTGTAATATACAAGTCCCTCCCTGTAATGTCTACCCTTGGAGGCAGGTTTCAAGTCCTGG
ATACTCGACATGCCCAAGCAGAGGCTGTGGGACCTCCCTTAGACTTCACTGGGGCCACAATACCCACTCCCTGGTCCCTGGACCAGCAGCATCG
GCATCACCTGGAGCTTGTTAGAAATGAACTCTCAGGGCCACCCACACCTACTGAGTCTGAATCTGCACTTTCTCAAGGTGCCCCTGGGGTCTCA
CATAAACATCAAAATTTGAGAGGCACTGGGCTAAATGTCTCTAAGATCCCTCATGGTTCTTAAGATTTAATGATTTTATAGAATAAACTGTATG
TTCAACTGAAAATTATCAGTTCATATGGTGACATTATCAATTTAAAACACCTTGGGATGTTACATGTCCACACAACCATGTAAGTATTTGTTGC
TAAATTAGTTTTAAATGGATTTGTCCAGTTTCCCCCGATGTCAAAGTTACGTTGCTTATGTATTTCGGCACCTATAAGAATTAAAATATTACAT
AACGTTGTATTTTCTTCTTTCTAACATACAGGATTAGATTCTTGCTTTAAAATTCTCCATTAATGTCTTCAAATTTTTCTTTTATATTCATTGA
AAATTCAAAAGCAGATTAGGGTTCTGAATACCTGTTAAGAATACTCAAAAGGGATATTTAAATAAATATATCTCATTAATACATTGATTTTATA
TTTAGCATATTTTTTCTTCTTATACATAGTCCATATTTTGACCTTATGGGAACCTTTGAAGTCTGAGTATTTTATAATGATTCTCCACAATACC
TCTTTATCTTTTAGATTCTAGATCTGAGATCAGGCAAGTAAAATAGTTTTACCTTTTTCCCCTCAAAAGGCCATGACTAACAAGCTCATTTACT
TTTTCCTTAAATATTTTGCTAATTATCCTTTTAACTGATGAAGTTCAAGTGGAATCCATGATTCATTTAATCTTTTCCCCTTTATCTAATTAGT
CACTTGTTCTTTTGATGTTCCTAGCAAAACTAATATTTAAAGGGCTAGTCATAAAATTCCAAAATAAAAAGTTCTTAAATGACTTAAAATAAGA
GATAAAATATGCTTTACTCTATAGTAAAAATGGAGAAAACGTCTCATGAATAGATGTTCTTTAAGGCTGTGAGTTATTTGATTTAAGCGTGTGT
TACCACGTCTGTGTTCTTTTGGTATATAGGACCAAGAAACTACTTTATCGCTTAATGTTTGCTGTACGTATCTCATCTAGATGAGATAATGTAT
GACAGAGGGCTTTATAAAATGATAAAGTAGTGTAGACTTTTTTTTTTTTTTTTTGAGACGGAGTCTCGCTCTGTCGCCCAGGCTGGAGTGCAGT
GGCATGATCTCGGCTCACTGCAACCTCCACCTCCTGGCTTCAAGCGACTCTCCTGCCTCAGCCTCTTCAGTGGCTGCGATTACACCCATGTGCT
GCCGTGCCCAGCTAATTTTTGTATTTTTAGTAGAGACGGGGTTTCCCTATGTTGGCCAGGCTGGTCTGAAACTCCTGATCTCAGGTGATCCGCC
CACCTCAGCCTCCCAAAGTGCTGGGATTACAGGCATGAGCCACCGTGCCTGGCCGTATTCTTTTTTTTTTAAGACCAAGATTTAGGAAGATTAC
TGCATATTGCTATAGCGTTTGTTCGATTTTACATTTTTTCAGAATGGTTTGAAATCACATATTCTAGCTTGAACTCTAAACCTGGTCTGTCCAG
TATGGTAGCCATCAGCCACATGTGGCATTTGAGCATATGAAATGTGGTTGAGATGTGCTGTGCAATATACACCAGCTTTCAAACAAAAAGGATA
TATATCTCCTTGATAATTTTTATTATGTTGATTACATGTTCAAATGATAATGTTTTACATATATACGATTAACTACTAAATATTAAGATACATT
TGCCCTGTTCCTTTTTACTTTTTAATGTGGCTACTAGAAATTTAAAATTATATGTGGTTCACCTTATACTTTGATTTCAAAGTGCTGATTTAGT
CTCTAAAGTCTAGAGTGATTGAGAGCTCTCAGAGAAAAGAAGATCAGAGCTTAACAGAGAGTTTTAGAGATGAGGTGCCACCGACAGAATCCAC
TTACTTGAAGAAATCCCAGTCTGCACCTTTGCCCTACTTTTACAGATTACTGCTTTCTTGAACCCACAAAGTAGAGCACTGGGTAGCAAGCATT
TAATATCTACAATGTACAGAGCATTGAAGGGGGATTAAAATAAGTATCACACAGGTCCCTGCCTTTAGGGAAAACATATAGTCTACTCGGCAAG
CAAGATATGAGCACATGAGTTCTTAAATCATCGCTTGAGAGATACATAGCTGTTCAATGCTCACCTCCAAGACTGTTGCAAACACACCGTTGAG
GCACTAAAAAAAGTGCTTCAAAGGAAGGAAAGGTCTCCACGGCCTGGAAAGGTCTCCATGGCCTGGAAGGGTGTCCCACCAGAGGAATGCCTTG
GCTGGGCACTGACGAGTGGGTAGAATTTCAGTGGGCAGAGAGAAAGGGGGCATGGCCAGGTAGGAGGCAGGGTAGAGTCGGAAGAAGGAGGAAA
ACATGGAAAAGCACAGAATGGTTGAGAAGTCAGTCACAAGAACAGATGGATTGAACTGTGAGCTCTGAGTACAATCATTCTCTAGTCCACTTAT
TATTGAAGCTTTAATATTTGACATCGTCTCCTATAATATTATGTATCTTGACACTCTTCCGAAAATTATTTACCAAGTGTCTCAAAGTAGAGGT
TAGGGCAGGACACTTTAACTTTGGGTAGAATAAATGTTGCATATCTTTGATTGTTAGGAAGTTCTTTAAATGGCAAAACTGATAATATTATCTT
GCCAGTTATGTTTATACTAACATTTTGTGTAGAACCATGTAACTTTAAAGGCTTCTCGGATAAGTACATTATTACTATTTCAGGCAGTAGTGCT
CACCATTTGTATATTCTTCTTCCTTAAAATGCCTTCAGTGTTATGTCATTTGATCTTTGCTCTTCGCTCCATTTTATAGAACTGCAACCCAGAG
AAGCTAAGATTGAATAATTAATGGTAGAGTCTGGAATTTGAACCTAGCTCTTAAGCTTCTTCCATTTTACTGTGTGAAACACACTAGTTTTCAT
TCCTGTAAGTCTGTTTCTTAAGAGAAGCTGATTAATTTTATTTCTTTTTAATTGAATGATAATATAAATAGGGAAAAGTATGCATATAACATAT
ATTCATCTTACTGACGAATTTCCATAAAGTGAACACGCTGTTGTAACCAGTTCCCACATTAAGACACAGCATTATCAGCGCCCTAGAAATCCCT
CAGGCTTCCTTCCAGTAACTACTTCACCCGTGGTAGCCACTACTCTGACTTGGGAAACCATAGATTTGCTTTGCCTGTTTTTGAACTTTATATC
AATGGAGATACAGAATGTACTATTTTGTGTCTAACTTCTTTCACTCGATAATATGCACAGTTAACTCATATTGTGCATGACACCATATTGACTA
TGGTAGTTGATTTTTCATTCTCCATGCTATATAGTATTATATTGTGTGACTATACCACTATTTATTAATTTGTATAGTTTCCAGTTTGGGGCCA
TTATGAGTAGCAATGTGAAGATTCTAGAACATATCTTCAGGTGAACAACATAATTATTTGTGTTGGGCATATTTAGGAGTTAAATCAGGAAAGC
AGTGTAATTTTTAAATGTCTTGTAGATGAGGCTGAAATTTGACTTTGTGACACTCTTCAAGAAAAGAAGTGAAAACCTCAAACTGTTGGTGTCT
TGAATAGTATATCAAAATAAATACTAAGGCAGCCATTATGAAAAGGTCCAAGGTAGACGAATTTTAATGCCCGAACACATTTTTTCAAGGGAGG
ATGAAAATAGTCAATTCTCATATAGAGAGAATTTGAAAGGGTTAGGATGTTAAAATACTGACGTTATATCACTCAGGTTTCTCTTACAGTTATA
TTTTTTTTCTTCAAGAGAACTTTTAGCAGGTGATAGTGGAAAACTAAGGTGTGATTGGTACCAAAATGACCATTGTCCCAGGGTATAATAAGGG
ATGAATAACTGGGGAGTGTATTTACTAGGAAATAAGAAAATGCCGTTGGGTTAAATGGCAGCAAGGTACATTCCAAAATCTATAATGCAACCTA
TGGTTCTCAAAATTTTACTGAAATTTCCTCTCTCTCTCACTCTTTTGCTTAGCGTAATCTGTAGGCAAAAGAATTTGCATTCTTTTAAAGAAGG
CAATTATAAATACCATAACTTCTTTTTAGAAAAAATCTTAGACGTTTATCCAGAGCCAACCTGTCTGAATGAAAATGTAGTTCTAGGATTTGCC
AACACAAAAGACTTCAGTGTCTACCTTTAATCCTTATTAACATTAATGGTGATCATTTAAAGGTGTGTGTTTTATGTTTAGACTTATAGGTGTT
GTTGATCTTAAGCAGACTAGGCAGAGAAATCCTTCACATTTTCTGTGCTCTCATTCAGGATGGCCCTAGGCATGTTACATGGCAAATGAGAATA
TGTGAATAAATACTCTCATACTTTCTTGGGCCTCAATCAAAAGAACATTTGTTACCATTTTTAGTCATAAATTAAAATTATTTCAGAGTAGGAA
ATATAACATTAATTTAGAACATGGCTGTCATAGATGAAACGTTTThCACATGTATTGCTGTGGTAGACATTCCTTGCATTTATTGAAAGCCTCT
CCTTTATAAAGTTAGTGAAGTGTGACTACTCTGACCTCCAAATACTGGGTGTATTTGAACTATGAAAGTAGGTTTAAAACTTACACTTTTCTTT
AATGCCCATTGATGATTTCTTTCTTTTTTTTTTTTTAAGACGAAATCTAGCCCTGTCACCCAGGCTGGAGTGCAGTGGTGTGATCTCGGCTCAC
TGCAGCCTCTGCCTCCTGGGTTCAAACTGTTCTCCTGCCTCAGCCTCCCAAGTAGCTGGGACTAAAGGTGCACACCACCATGCCTGGCTAATTT
TTTTGTAGAGACTGGGTTTCACCATATTCGCCAGGCTCGTCTCGAACTTCTGACCTCAGATGTTCAGGCCGCCTTGGCCTCCCAAACTGCTGGG
ATTACAGGCGTGAGCCGCTACGCCCATCCAGAACAACAGGCACTGGGGCCTACTGGAGAGTGGAGAGTGGGACGACGGGGAAGAGCAGAAAAAA
AAAAACCTATCAGGTACTAGGCTTAGTACCTGGATGACAAAATAATCTGTACGACAAACCCCCATGACCCAAGTTACCCATATCACAAATCTGC
ACATGTAACCCTGAAGGTAAAAGTTAAGTAAATAATAAAATATTTTACATTTATTTAAAAATTCTAAAATGGTTGTATTTTAAAAGTAAAAACA
AATTTTGGTTAGGATTTGAGCTTATTCCATTTTTCTTCTAATTTTCCAAATTAAATGTAAAATGAGAGCATCCCAGGTAGGGGGAGGTAGGGCA
GAAACCAAATAATTTTCAAGTCATATTTGAAGAGCAGGTATGTTCTCTCACGATTTACAGTGCAGGTGCATTTTAATTTAACTGCTTTAAATGA
TGATAAAAATTGTTCAAGTAGAACAAAGCAAAGTTTTAGAAGTGACTTGTAAACAGCCATTCTTAATTTTTTCCTGTTTTCTCTCCACTTATGT
GACTTCCATCAATTTCTGAAGCAGTCCTTTCAAATGACACTGCTCCAAACATCTGAATTGCATTCATCAACAAAATCCAGCCTTTTCCTTTGTA
CCACAATGTCGGCTCACGGCGCAACCCGTCTTTTCTTTGTCTACCCTCGTGCCAAAGCAAAGACTACTCCTCCCTGCGTTTGGACCCAGTGACA
TGTTTCCATTTGTCATTGAACACTAGGGGGAGGAACTGTAGCCTCTGAGGTACCCCCATTTGGTCCTCACCCTCTACCAGAGGATTTGCTGCTT
CAGCCTCTTCAGTGCCCCAGGTATTTGATGCAAAGATTATCCACTTATGCTGCCACTTATCAGGGTTGTGTGGGTGTCTTTAGAGGGGTCTCCC
CTTAGCATCAGGTCTCAATGACAGAATCTAAATAACACAGGTTACTCATCACGAGTTAAAGATTTTTTCCCTTTTCACATTGATGCTTCTGATA
TACTTTTATACGTTAGCTGATTGTATGTGCGTACTTTGTGGCTGCAAAGATGCAGCTGGAAAGTGTGTGGGGGTTGTCTGCTTCCTCTGAGGCT
GCTACAGGTGGTCAGCTGCGGGCGGCTGTGATAAACGTACAGCACCCACTTCCGCCAGCTTGTCTTGTGCGCCCTGTAACCCACTGCATGAACC
CGAGTGGCACTTCAACCGCATTTTCTTGTTGCCACTTTTATCTTTCACTCCTTCTCACTTGCCTGTTGACGTTTATCACTAACGTTGTGATTAA
AAATTCCCTTTTTAGGTATGAAAAGTCTTTTCAATTACTTTGTGTTCCTTTTTTTGTTTTGTTTTGTTTTTGTTGTGTGTGGACTTTCTGGGTT
TTGTAAAAATGAATCTTAAAAAAATCTTAATATTATGTAACAGTTAGATAAAGGTTAAGTAGCTAATATATTCTCTCATCAGGGCATACAATCA
ACGTATGTTCTTGGAAGATTAAGTGTGTCAAAATGCATTTTATGATGGAAGCACTGTCAGTTGTTTAGAATAGGCATGGAAAAGCCCTTTTCAG
AATATTGTCATAAAATCAGACTTGGAAAGGTTTTCGTTTATTCAGTCTGTATTCCCCTATCTGTCTGTTTAATCTCTGCATATTACTGTCCTTG
GAGCACGGAGAAACGTGAGCTACCCTTGGAGGTTTATAGTCCCACTGGAAGGGAACTAATGTTTACGCCTGATTATATGATAGGAGCTTTACAT
GTTCTTATTCAGTCTTTGAGGAAACCTTATAAATAAGATAATAACTTTTTCAGAGAAAGAAGCTGGGGTTCTGAGACATTACCCACTTAGGGGC
ACCCAGAGGAAAGAGCAAGACCTCCAGCTCCATCACCAGTAATTTCCAGCCTGTGAGCTTCCCTCCACACTCAGTAATTTAAAAAACAACTTGA
TTTAGGGACATCTAGTAAATATTCTAGAAGTTCAGAGCCAGAAAAGCTAGTTTGCAGCTGAAGTACTCAGATTTCCCACTGGCTGAGTGGGAAA
TCAGACCCGAGCCTTGACAGTGGGCAGAATTTGTATAGAGGAGCCAAGAGAAGAACAGTTTCCACACTGTATAAGCCAAATCATGGAAGGTGTA
ATAACAAAGGAAGGGTGAGCACACAGGTTTAGGAGATGCTGTGTGGGCCAGTCCAGCTGGGAGCCATTACCATTTAACACTTAATCGAGTGCTT
ACTGTGCTAGGCACTCTTCTAAGTTCTTCACACCTTTTAACTATTCTTATCTTCACAGCAAGCCTTTGCAGGAGATATTATCAAGTCCCTCCTG
CCCCCACTTTTTTTAGAAACAGAGTCTTGCTCTGTTGCCCAGGCTGGAGTGCAGTGGTATGATCGTAGCTCATGGTGGCCTCGAACTCCTGGGC
TCCAGTGATCCTTTTGCCTTAGCCTCCTCAGTAGCCACGACTACAGCTGTGTCCCACCATGCCTCGCTAATTTTTTTTCCCCATAGAAACAGGA
ACTCGTTATGTTATCCAGGCTGGTTTTGAACACCTGGCCTCAAGCGATCCTCCGCCTTAGCCTCCCAAAGTGCTGGGGTTACAGGTGTGAGCCA
CTCCACCTGGCCTTCATTGAGGCATGAGGAGACTGAGGCACATAGCAGTTAAGTTGTGGAACTGTGATTCAAAGCATCGCCACCAGGATCCAGA
GCCTGAGTTCTTAGCTACTCTCCTGTCTTGTTTTCTCTCTACTCGGCAATGCAGGTTGCTTGTTACACGGGTGATGAGGATTCTCAAATTGCTG
GGGATAGCGAGAGGAAGATGAAAATGGCAGGGGTAGTGATAGAAAGATCAAAATGGCAGCAGCACGCTGGCCAATTTTACAAGAGGAAACATTA
GGAAAGAAGAGAAGAGGGAGTTTTTCTCAGCTAAGCAGGATCTGGTCACTGTAACTGCGTAGCAAAACATAGAGGTGAGATGTAACAAATCAAG
AGCCCTTGATGAATGACTGGAATTCAAGAAGAGGGAATGACCAAAAATGAATCTAAGATTTGGAGCTTGGTTGGCTGGGAGGATAATTCTGCTA
TTGCAGAAATCAATGAGGTCAATAGACAAGCTCATTTTTCCACAGCAGTAGTGTTGCAGCAAGAAATCAAGGCTTTTCAAGAGCATATGCAGTA
GAGATACCATATGCAGGAAAAATGGCTCACTGGAGAGTTGAGAATAAACGACAGCAGTTCAAGGGAGGGAATGGGGATGATTGGGGGGCTATCT
GCAAGGAGAGAATTGATGTGCTGGGAACAGACGATTTCTGAGGGAGAGAGAAAAACCCAGCGCCTTAAAAACTGTCCTTGGTCAAGAAGCAGAA
GGAGAAAGAGGAGCAGGAAACCATAAAAGAAACAGGCGAAGCAGGAGAGAGAGGAGAATAAAAAGAGGAAAAAAAAAACTACTGAACAGTGTCA
GACACTACAAAGAAGTCAAGGAGAACAAGAGTGAAAGGGGGACATAAGATTTGACACGGAAGAAGGCCATTTGTACCCTGCACAGTAAGTCTTG
GTGACATGAGGAGGTGAAATCCAGTTGCATTAAACTCTGTGGTATTGCTGACTTCTTCCTCATGCGCAAGTCCCTGGACCTTTTCTAAGCACTA
AATTTGCAGGTAGTTCAATGTCAACCAGAGAAGGAAGTTGGTTAACAGCACTTGGTTGCATGATACTACGTTTTGTTATAGTGACCTAACTGAT
CTAACGTGTTTTGTCTTTCAGCGCGTTCGTTCACATAGCTCCCAGTTTTAACATTTCGCCACCTACTGAAGACATCATTTGGGACCAAGCTGAT
GACCCCTTGAACCACAGCAGGTGACCCAAGGACCTCAGCTTGCTCTTTGTTTCATAACTTTACACAGCTAGCTTACATTCCCACCAGCAGTGTC
AAAGTGTTCCCTTTTTACCAGATCCACGCCAGCATCTATTATTTTTTGTTTTTCAGTTATGTCCGTTCTTACAGGAGTAAGATGGTATCTCGTG
CACTTTTAATTTGCATTTCCCTAATAGAGATGTTGAGCATTTTTTCTTATGTTTCTTCGCTATTTGGATGTCTTCTTTTGAGAATTGTCTGTTC
ATGTCCTTTGCCCACTTTTTGATAGGTTCTTTTTAAATTAGTTTATTAGCTTTTAATATAAACATGAGAAAAACCACATAAATTGTAGCATTCT
TTTCAAAATAAAACTGCAATCAATACTTGAATGAATCTCCAAGCTCTGAACTTCATAAACAATGTTTTTTCTTTTCTTTTTTTTTCTTTTCCTT
TCTTTTTTCTTTTTTCTTTTTTTTTTTTTTTTAAATCGGCTTCTTTTTATCTTGCTGATTTGAGTTCCTTGTAGATTCTGGATATTAGTCCTTT
GTCAGGTGCATAGTTTGCAAAGATTTTCTCCCACTCTGTGGGTTGTCTTTTTACTCTGTTGATTTTTTAGTTTAATTAGGTCCCAACTATTTAT
TTTTGTTTTTGTTGCATTTGCTTTTGGGTTCTTACGCATCAACTTTTTCCCTAACCCAATCTCTAGAAGAGTTTTTCCAGTGTTCTACAGTTTT
TATGGTTTCAGGTCTTAGATTTAAGTCTTTGATTCATCTTGACTTCATTTTTGTATAAAGTGAGAGATGAGGATCCAGTTCATTCTTCTACATG
TGGCTTGCCAATTATTCCAGCACCATTTATTGAATAGGGTATTCTTTCCTCACTTTATGTTTTTGTTTGCTTTGTCAAAGATCAGTTGGCTGTA
AGTATTTGGCTTTATTTCCGGATTCTCTGTTCTGTTCCATTGGTCTATGTGCCTGTTTTTATACCACCACCATCCTCTTTTGGCAACTATAGCC
TTGTAGTATAGTTGGAAATTGGGCAATGTGATGCCTCCAGATTTCTTCTTTTTGCTTTTGTTCTATTGGCTATCTGTACCTTTTTTTGGTTCCT
TGTGAATTTTAGGATTGTTTTTTCTAGTTCTATGAAGAATGATGATAGTATTTTGATGGGAATTGCATTGAATCTGTAGATTGCTTTTGTCAGT
ATGGTCATTTTCATAATATTGATTCTACTCATCCATGAGCATGAAATATGTTTCCATTTGTTTGTGCCATCGATTATTTCTTTCAGCAGTGTTT
TGTAGTTTTCCCTGTAGAGATTTTCACCTCCTTGGTTAGGTGTATTCCTAAGTATTTTATTTGTTTATTTAGCAGCTGTTGTAAAAGGGATTTA
ATTCTTGATTTGATTCTCAGCATGGTCACGGTTGGTGTATAGCAGTGCTACTGATTTGTGTTCATTGATTTTGCCTCCTGAGACCTTACTGATC
TAGGAGCTTTTTGGATGAGTCTTTAGAGTTTCTTAGGTAGTACAACCACTCTGGAAAACAGTGTGGAGATTCCTTAAAGAACTAAAAGTAGAAC
TACCATTGCATCTAGCAATCCCACTATTATCTACCCAAAGGAAAAGAAGTCATTATATGAAAAAGACACTTCGACATCCATGTTTGTACCAGCA
CAATTCGCAATTTCAAAAATATGGAACCAGCTTAAATACCCATCAACCAATGAGTGGATAAAGAAAATGTGGTATATATACATCATGAAATACT
ACTCACCCATAAAAAGGAACAAAATAATGGCATTCACAGCAATCTGGATGGAATTGGAGACCATTATCCTAAGTGAAGTAACTCAGGAATGGAA
AACCAAATATAGTGTATCATAAGAATGATATAATGGACTCTGGAGACTCAGAGTGAAGTTCCACTAGGGAACTTATCCATGTAACCACAAACCA
TATGTTCCCCCAAAACTATTGAAATAAAATAAAAATTAAATTAAAATTTTAAAAGTTCACACAGCTGCCCAGCGTGTGTCCCACTCACCGGGGC
CATCACCTTATTCTGTGTATTTCTAAGCATCTTATGAACACACTGATTCTTATAAGGAATCCAAACAATATAGCACTGCCACACTTGGGGCTGC
TATTTGTTTTAGGGCTCTTTTATTATGGAAAGTCCAACCGATATGAAAATAACCTAAATTTTCTCTGACTTCTCAGAAATTCTCAAAACATCAA
AATGTGCCCTGTCTTTTGATGCTGGTTACCCCTTGGTGGCAATCCAGGATTTCCTTATTTCAGTCTCACAGCTGTGAGTTGTAAAGCATTTCAT
TCACCACAATGCTAGTTACAGTTGATTTTATTTAAGCTGCCAGATTTGGAAATGCACCTCACTATAGCATTAGTTTAAAAAATTTAGGAAGGGG
AGAAGAAATTTCTATCTACAAATGATATTTTTTACTTTTCGACTGTAAGTATGTTTTTTGCTTCTTTATTCTTCCCACCACTTTTTCTGGGTAA
CTGTAACTTGGTGTATCAACTTTTAGAAAACTCACAAGACTTAGGAGTATGTAAGCTCTCTCCCTTCCCTTCCCACCCCTCACCCCCAGCATAG
AATATTGGTACGTGAAACTTTATTAAATAGAATCTGCACGGAGTAGTCAGACCTTAGCCCCACCTAACCCTGCCACTTGTCTCTGTTACTGTTT
TCTGTTGCTATTAGCATCTTCATTCAGAAGGACGAGAGGGGAGAAAAAAATGAAAATGAAGTTAATTTTGTTCAGCAAAAGTTAAACTCTCTGG
CTGAACTCTGGGTAGCTCCTTTTGGAGTTGTATCGGCTTTCATTTATTCTCCAGGTCTCTTAGTCTTCACTTGAAAATAGAAACTAGAGTTCAT
TTTTCCTGACAAGAATGAACTTAATTATTTACCTAGAATGGTTAGTAATCTGTTCTTATTACCAGAAAGCTTTCTTCTGATCTATTTAGTATCT
TTTTTCATAACAGTTTGAATATATTAGCATGTATTCTCAATAGAATCTAATCTTTCATTATTAAAGTTCCTAAATATCTTATATTACTATGTTT
TATCATTATGAACATTGACTCTCATTGTTTAGACTTTTGCTTCTCTAATTATCTTGGGTGAAGGACCAGTTTTGTTTTTAATTTCCAGTATGTC
ATGGATTGATAATTTTATAAAATACAATAAAAATGATTTACTAGAAAAATGAAATACTAGAAAAATGAAATTTAAAGAAACTAAAAACATAAGC
CTGATTAAACATTTATTATTCTTATAGTCAACAGACATAAAATTACTCTGGCAAGCTGCTGTAAAAAAATTCTAAAGGTCTACTCTCAGTTTCT
GTGCTTATTCTCGGACTAGGCCTGGTCTGCAGAGTCCAGTTCGAGTTGCACTGTTGGACAGAATATATACAACTTAGCACACTGAAGTTCAGTA
GATACTAATTTCAGAACATATCCTCAAAGTGGGTTTTTGGGGACTGTCCCCTTCACATTTCTTGCATGTATAAAAGGCAAGTGCAGTTCGGGTA
CAGTGGTTCATGGCTGTAATCCCAGCACTTTGGGAGGCCAAGGCAGGCAGATCACTTGAGATCAGGGGTTTGAGGCAAATTTGGCCAACATGGT
GAAACCTCGTCTCTAAAAATACAAAAATTAGCTGGGCATGGTGGTGTGCACCTGTAATCCCAGCTACTCAGGAGGCTGAGGCATGAGAATTGCC
TGAACCCAGGAGCCAGAGGTTGCAGTAAGCCAAGATCATGGCACTGCACTCTAGCCTGGGCCACACAGCAAGACCCTGTCTTGAAAAAAAAAAA
AAAACAAAAAAGGCAAGTGCAGAAAAAAATGCTGTTTACTTAAACTTTTTCTATTTTAAAAAAAATTGTAGGACAATGCATAAAATAATATTTA
CGTCAATATAGAAAAAGTTGATTCTCTTTCTCCCACTCCTCTGCTTCTTTTCCTTTCTCTATACATCATATACATACATACAAACATATACCTT
TACATGGAGAAGGGACATTTTTTCTGCTATGAAAATGGCATCATAAACAATACTCTGCAACTTTCTTTAATATTTTGTTACAACATTTATCCTT
ATCAAATATGAATCTAGTGGCTCTTTTTAACAGTTGCATAATATCCTATGGTATCGTTGTACCATAGTTTATTCAATTATTCCCCATCTGATAT
ACCTTCAAGAGCATTCTAGGTTTTTACCATTCCTAACAACACTGCAGTTAAATTCTTGTACATATATTCATGTGTATCTACTTTGATATTTGAA
GGATAGATTCCTAAAAACGGAGTCAGTGACTCATGCATCATGTCAGATGATACTCCAAAAAAGTTGCATTGCTGTAGAGTCACACCTGCAAAAT
ATGAGAGTACCTGTTTGCCTCACCCCTGCCAGGTGTTAGGAGTCTTTTTAAGTGTTCTCTCTCTGCGGTGTAAACTGATCACTAGCTGTTTTAA
TTTGAGTTCCCCTGATTGCCAGCAAGAGTGTGCTTCTTTTCATATGTTTAAGGGCCTGCTGGATCTTTCTATTCAATTTGGGGGCAACAAAACT
TGACAGATTTTATTTTATTAGGTCTTTTGTGTACCAAGTTTTCTGTCTGTGGTACACTCAGTCGTTGGCCTTTATATTATTTGTTCGTTTTAGT
AGGGTAGAGGACATGGGAAAAGAACAAAGATACAGTGTTGAGGGTTTTTTGGTGGGGGGGAGATGTCTTAGTTCTGATTTTCTTTGTCTAACAT
GTTTTTTTTCTTCAAGATCAGCTTACAGGAAATCCGTCCCTATTTGTGTGTAATGTAAAAAGGGCACAGGGTGGGGCATCTTTATAGCTTGGCA
TAGTGTATATCGTTATTCAAGATTGCTCAAAGACTATGCGTTGCCATGTGTACTTTTAAAAAATTCAGCTAACTTCATATAGAATTAACCCTCC
TCAAGTGTACAATTCAGTGGCATATAGTACATTTACACTGCCGTGCGACTGGGACCTCTATCCAGTTCTGAAACATTTTCCTCACCCCAAACGA
AACTCCGTCCCCATTAAGCAGTCACTCCTCATTCCCCTTATCCCTCAACCCCTGGAAACCACTTCTCTCCTTCCTCCGTCTATGGTTTCACCTT
TTCTGGATATTTCATATAAATTGAGTCATGCAGTATGTGATTTTTTATGTCTGGCTTTTTTCATTTAGCATAACATTTTTGAGGTTCATTTGTA
TTGTTGTGTGTATCACTACTTCATTCCTTATTGTCGCTGGATAAAATTTTATTGTATGCTACAATATACAATAATTTGTTTATCCATTTACCAG
TTGATATTAGGGTTGTTTCCACCTTTTGGCTGCTGTGAACAATTGCCCATGTCCACCTAGATGCCAAGTTAAGTCATAGAGAGGCATGAGGGAA
GAACAAAGACAGCTGAAAAGAACCTCCCAGGGAGCATCTTCTGTCTTCTAGAGCCTTCCTCTCTCTGTAGTTCGTCATCTACAGGCTCTTTAGG
AGGCTGGGTCAGTCGTGGGGCCAGATTTGGGATCTGTTTTCACTGGCTTCAGTATCTTCGTTAAACCAGAGATAGACCCTCCATATCACACCCT
CTCGATTTAAAAAGGAATTTACATCCCACCCTCAGGGCACTAGGTCTGCTCTAGCCTCCATGGGTGCAGTGCCCGCAAGGAACATTCATGGCTA
TTCCTCCATGGTTGTTGCCTCTACCTTTAGCATCTGGGTTGGAGCTTGTAGAATGTGGTGATCAGGAGGAATTGTGCCCACATAGTACCTGGGC
ATATGACAGTATAGGCTCATTTCCCATCTCTCCACCCTTAGTCCAGTTATGTATCTCTCAGTTTTCTCCCTGGTAAAATGGCATTACTAATACT
TACATCATAGGGTTGTAGACATAGTTACATGGGATAATGTAThGAGAGCACTGTTACAGTACCATGTCGTACTACTTGATAAATGTTCATTTCT
TCTGTTCTCAGTGCTTCTCTGTCCTCTTCTATCTGGTGCCCAGGACATTACATACTTCACAAGGTAGTATTAAGTTTAAAATGAGTTGATAATG
CAGAGGAAGTCTCTTAGCACCATGCCTGCAACGTAAGTCAACACTCACTATAGATAGAAAGGTACCTTACCTTGCAGTTAGGATTTCTCATTGG
GAAAATGCCATCCAATTGATTACAAGCCGTCTAAAGTGTTTTAAGTGGTAAAGATTTCACCTGGGTATATTGAGTTGTTCTCCAGCGTGGTGTT
TATTACCATAGACACATCGGGCTAATTAAGACAGCCATGTTCAGGATAGATCCACTATCACTAAGATCCTGATGTGTGAGTTGGGCATTTCCCA
TCATGTTAGCACAAATATATTAAAGAAAGTTACTTTTATATCAAAGTGTTTCCATCCCAAAGAAAATACTTGTACAGCCTAAAGTTCTCTTTCT
TGTGGAAGCTGACAAGAGACTTCTGTCTTGCCCTCCAAAGTGGTTTAGTATCCTATTTTGAGCACTCTTTGGGTGCTGTTATTTAGAAATTCTA
TTTTAGAGCAAATGGGAAGGATTTTATAAAAAAGGGAGAAGCAACTGTCCTCCACCATGCTCTCTCTCAGCTGTGTAACAAGCCTCTCTGAGGG
TGAAGTGGATCCCCATAATGTGGGCCCCATGTGTGAACCTTTTACAAGCTTTTCTTGTTGTAATGGCTGATATAATTAAAGGAGAGAGTTCATG
TTTTGTCTCAAATGGAGCATGCCTTGAGTTACAATGTTTTGTTTTTCTCACCAAATATATCCTCACTATAATAGCAAACAGCTTGTATAAGATT
TTTACATAAACATTTGAAAAAGCATTCTTGTCTTCACTGAAATTTTGAAAGCCTTTTAAGAATATTTGTAGCACTTATGTTTTTAAATGTACAG
GTCTAACATGCAGTTGTTTATTTGGTAAATTCATTCCATGAGGGTGCTTTGAATCTACCTCCAACCTCTCACGGGTCTCCCAGACTTCCCAGGA
ACCACTGCTTGACAGCTGGAGAGACAGCAGGTGAACCAGGGTCTCACTGGGAGGGAAAGACGAGGAAACCCCAAAGGGACAGCTTTCTGGAGAA
AAATGCCTGGCTGTGAATGCCTCTTCACCTCCATCCCAGATACACACTTCCAGGATGTGCAAATTGAGTCAACATCATGTCTGTAGAGGACAAC
AACATGTCTTTTGTTAGTCTTGACATTTCTTTAGCCCTAGGCCTCTCAAGTTTTGGATATGTGATGCTCAAGAAATGCATTTACAATTTGAAAA
CCTGGTGCTCATTAAGTAATTCTTTTAAAATTTTTTTCTTTTCCTTTTGAAAAATTCAAACCTAGAGAGGAGATGAAAGAATCATATGAAGAAC
ATTTATATACCCTTTGTTTATATTTGCCAATTATTAATATTTGTTATTTTGTCTCTGAATTTATAGTTTTGTATGTGTGTTGGCAAGAGGCGTT
CATTTTTTAGAACTATTTAAAAGTAAGTTGCAGACTTACTTTCATCACATTTTTCCCTGAAGACCTCAGCATCATTTCTGAAGAATAAGAACAT
TTTCCTACGTAACCACAATCCTGTTATAAAGTAAGTCTTAATTGAAAACTTCAGACAGAGGACAAAAGAGTAAAAAAGGCAGAGATTGAACAAA
GCTGTAATTGAACTTGGAAAGTATAAGGAGAAGAAATATAGGGAGGTAATTAGAACTCCTGTTTGGTTCCTTAAAGGATAAATTTTGTATATAC
CACCAAATCACTCAATGAATGCAGATTTTCTTCAAATATTTACTTTATCTAGTCCAAGGAGTTTCTCCTGGTGCTCTGAAGTCACTGTTTTGGC
CACGGTTATAACCTTTTCAGCCTGTAAATACATTTCTTCAGCATCATCAATCTAAAACCTGAGTGACCACATCCTACTTAGACTTGTCGTTAGG
AGCAAATTTTCAAGGGATTTTGCTGGTGCCATTACCTTGAGAACATGGGACCTTCAGTTATTACAGTCAGAAATGTTTACTAATGGGCAGTTAG
TGTAAACATGGGAGTCCTTTCCTGACATTTAATTTAGGTTTGAGTAAAAGCTGATGTGTTAGCACACTATTTTGAGAAATAATTTTACAGGCTT
GGCAAAATGTTAAGCTTGATGTCATCACAGACTTTTGCAGATTTAGGCTTCAGGAGTCAGCGCAAGAAAAAATGCTACTCTTATAAAGGCTGAA
CAGCAGGAACAAAGTTAACTGGATGGCATGTAGATGCATCACTAATTTGTCTATTTTAAAACAAAGATATACATTTTCTTCATACCAATAGCTT
ATAGAACATTAAACAAATTAAAAGGTTTATTTGGACTGTTTCCCTGAAAGCCTCCTTCTTCGGTAGGATTTAATATTTTGCCAGAAGAATTATG
TTTGGGGATTTATTTTTCAAATTTTTTAAAAAAGCTATTTTCACTAAGCTTTGGGAGGGGAACCATATTAAGCTGTCTGTCTAAGTAAGCACAT
CAGGCATCATCATGTCAAGAATGAGAAACTATGTGTTTCCTTTAACAGCAGTGTATGTCATAGACATGTTTGACCCATGATTTTTCATGAAAAG
GTCCATTTTCAGTAAACTCTTGTGCTTACCTAGTGTCTTCAATGTGATAACTGGCTTCCCACTGATCAGGGGTGAGATGCATCACAGAGAGTTT
GAAACTACACATTTTGTCTTTTATTTCCTTTTTCTTTTTACTCTTTCAGCCTTCAGTGGCCATCGAGATTTTTTATTTTAAATTCCAATTTTAC
ATATCAAATTTACAAACTGTGACTTTTAAAATAATTGTTGTACTCTTTTCTGTGAGTGAATTTTAAAAAGCATTTAATGTAAAATGCATGTGTA
GTTTCTTGTCTATCAAAGCCATCCTTTTCCTTAGATCAGCTTAATCGTCTCCTGTTTCCAATTTGTGTTAGAAAGTTCTGTGTGAAATGTGAAC
TCTAGTTTATATACTTACTAACAGTTAATGCGTGTCCCTTCGGATAATACCTCTTCAGCTATCTTTGTCCCTGTCTTACGTAGACCATTTGAAT
CTTTCTCCTTTAATGCCAAGTATCCACTTTTCACGTGTAAATTCGAGATGTCACTACCTGCACTTGGACACCAGTGTGTGCATGTAATGTTGAT
TGGTGTCATCATGGTTTTTGGCTACTCTGTGATAATTTTGTTTTTCAGAGGCAAATTTTGTGAGGCCAAACTTCGGAGATTGGGTCTGTACATC
ATTCATTCTTTCAGTCAGTAAACATCCATTTAGTCCCTACTCTGTGTCACTGCTACTCAAAAAGCAACAGCCCAGGATTGCTGTCCTGGAATCA
TGTGCACGCATCGTACCCATGAATGCGTACTAACAACACAACACCCACTTGAATCGCCTGGCAATCGGCCGTGGGGATGGGAAGGGGCTCCACA
GATAGGTGAATTGGTCTGTAAAAGTCATCGCTGTACAAATGAATCCCAAACCAAACATTGAAAGAATGCACAGTAGATTCCCCCAGTTAGGGCA
GGGTCATAGAGCATTTTGGGCAAAGAGCCCATACAAGGCCATAGAAGCATGAAACATATGCCACATTGTATCAATTTCAAATAACTTCATATAA
TGAGAGTTTGGGAACATGGAGAAGGTAGTGGAGGAAGATCAGACTAAAACTGATTATGAAGGACCTTACGTACCATGCCAAGGAGGCTCGCCTT
CATCCTGTAGTCAGTCGGGAACAACTTCAGGAATCTCAATGCAGAGCTTTCATGATGAAATTTGTGCTTTACAAATATTCTCTTGCAGCACTGT
GGAGGTACAGATAAGTAGGAATGGAATCCAACGCGACAGGCTCACTCTGCTCCTACAGAAGTTCATGGGGCAGTCCCAGTCGGCACATAAGGAG
ATGATGGTGCTGACACCCAGTTAACAAATAGAATCCTCAGAACTTGGTAACTGATTGGTTATGAGTATTGACACTACATGCGTCACTACATGAG
TGATCTCATCACTATATAAGTATATAAGTGCTCAATTAGATCGTGAGATCAAGGGGCGATATTAATGTAGGAAACAGAGAACAGAGAGAACCAA
TGATATAAGCCCTAGGAAGTGGACTTTTAAGGAGCAACCAAATAAAAACAACCTTTACAACCAATCATCTGAGGGTATGACGAAAACCTGAGGA
TACGGTGCTCCACGAACCCAAAGCAGGAATCTGCAGAAGGAGGGAGTAGTCAAGAATGTTGAATACTTCAGAGGGGTCAAGCAAGGGTTCCAGG
ATGTGTCAACTCGTACGTTCACTAATCCTAGCAACAGCAATTAGTGGGTGGTGAAAAATAAGAAATGAGATGGTTCATTTATACTACTCTTTCA
AGGAGCTTAGATACTAAGCGAAGGAGAGCAAGAGTTGTCAAGGAACATTTTTTTAATATTCTTTTTCAAGGTTAAAATCTCAAGCAAGTTTACA
TCCTGGAGGATAGATATAATCCATTAGACAGGAAGAAGAAGCAGATAACTGATGGAATAATGTCTCTAAGCAGATCTGATGGCACGTAAGCTAG
AACACATCGCAAGATTAGCTTTGGGCTAGACAAAGCACAACTCAGTTTTTAATAGATGGGAACAAAGGAGGAAAGACTGGGTGTGGATATGGAT
AAGGGATTGTAGGTCTTGAAAGTTGGAAGTTTGAGGGGTGTAGTGTCCATGAAAATAAGTGCTTAGATCTACAGCTAGAAGCGTAGTTGACTGA
TGGCCCAACTCCTCCCCCTCTGCAGCCACCCTTGCATTCTACTAAACTACAAATTGCCACTACCGTCTCCCAGTCATGACCAACCACAATAGGG
GCTCCTATTTCAGGCTCATTCCTGTGCGACACAGGACTTTTCCAGTGGGCCAAATTGGCTCAAGAACTCCTATTGCCCTCAGCAGACCTTTCTT
GCAACTGTGTTGGAATCCAAGACTTCCTAGCCATTCCTTCTTCCTTTCCTCTCCATGTCCATAGGTGTGGTTTGAAGGACCTCCCACATTCTGT
GGCTTCCTTTCTCAATATATCTTTTGTATTTCTAGTTCTATCTTCGTACTTGCTTCTGGGTAGACCCAAACTAACATAAAGAATTTGGTCGAAA
TGTGTATATTATTAAGGTAATGCTAGCTGCTGTAAAAATAACTTCTCAGTTCAACAACATGTACATCATTCAGACAGAAAATTAGTAAGAAATG
CAGGACTTGAATTGCATTTTTGGCCAAATGGACCCAATAGACATCTACAGAATTTTCCATCCAACAGCACCACAATGTAAAGTTTTTCTAGAGT
ATACATAGAACATTCTCCAGGATAGACCATATGTTAGGCCACAAAATTTAAGATTGAAATTATGTCTAGTATCATTTCACATATAACTTCAAAC
CACAGTGCTATAAACTACACATCAGTGACAGGAGGAATCTTCAAAAATTCACAAATATCTGGAACATAAACAACATGCCCCAGAACAACCAATG
GGTCAAACAAGAAATTAAAAGATAAATTTAAAAAATGCCTTGAGACAAATGACAACGGAAACACAATATACCAGAACTGATGGGATATAGGAGA
AGCAGTTCTGAGAGGAAAGTTTATAGCAATATATGCCTACATTAGAAAAGAAGAAAGATCTCAAATGAATAGTGTGACATTACACCTCAAGCAA
CTAGAAAAAGAACAAACTAAAACCAGATTAGTAGAAGGAAAGAAATAAAAATCAGAACAAACATAAACTAAATGGATAATAGAAAAGCCACAGG
AAGAATCAATAAACTTAAGAGTTGGGTTTTTTTTTAAGTAAAATTGGCAAACCCATAGATAGTCTAACAAAGAAAAAAGAAAACTCAAGTAAAA
CCAAAAATGAAATTGAAGAAATTACAACAGACACCTCGGGAATAAAAAGGATGATAAGTCCCTATTACGAACAATTATATCCCAACAAATTGGG
TGGCCTAGAGGAAACAGATAAATTCCTAGGAAAATACAACCTACCAGGATTGACTCAGGAAGAAATAGAAAGCCTGTAACAGACCAATCACAAA
TAGATTGCAGAAGTAATGAAAAACCTCTCAACAAAGACAAGCCCAGGAACAGATAGCTTCACAGCTGAGTTCTACCAGACATTCAAAGAATTGA
TACCAGTACTTCTTAAACTCTTCCAAAAAAATAGAACTAGAAGGAATACTTTCAAACACATTTTATGAGGCCAGGATCACCCTGATACCCAGCC
AGCCAAAGACATCACAAGAAAAGAAAATTTAAACCCAATTTCTCTGATGAATATTGGTGCAAAAAGCCCCAGTAAAATATTAGGAAACCAAATC
CAACAACACATCAAAAACATTACATGTCATGACTAAGTGCGATTTATCCCTGGCATACAAAGCTGGTTTAACATCTGCAGATCAATCAGTAGTG
ATACATTGCATTAACAGAATCAAAGATAAAAATCACATGATAATCTTAATTGACACAGAAAAAGCATTTCACAAAGTCCAACATCCTTTATTGA
GAAAAATGCTCAATAGTGTATGTATAGAAGCAAAGCTTCTCTATGTAATAAAGCCCATTTATGAAAAACCCACAGCTAACATTATAATACATGG
GGGAAAACTGAAAGTTTTTCCATGAACGTCTAGTATCAGGCAGGCATGGATGCCCACTTTCACCACTTCTATTCAACATAGTACTGGAAGTACC
AGCAATAGTAATTAGATAAGCAAAAGAAATTAAAGGCATTGAGGTCAGGAAGAAGTAAAGTTATCTCTATTTGCAGATGGTGTGATCCTATATT
GTAGGGGGGGAAAAAAGCCCCTGCAGATTCCACACAAAAAAACTGTTACAACAACGAATGAATTCACTAAACCTCTGGGGTGCAAAATCAACAT
GCAAAATCAGTTGCGTTTTTTTTCATACCAATTACAGTCTGTTTGAAAAGAACATGAAGAAAATGGTCCCACTTAACAAAGCATCAAAAAGAAC
AAAATACCTAGCAATAAATTCAACCAATGTGAAAGATGTGTACACTGAAAACTATAAAACATTAATGAAAGAAATTAAGCACAAAAAAATGGAA
AGATATCACATGCTCATGGATCAGAAGAATTAATATTGTTGAAATGTCCATAGTACCCAAAGCAATATACAGATTCGGTGCAATCTCTATCAAA
ATTCCAGTGACCTTCTTCACAGAAATAGAAAAAATGCTTCTTAAATTTGTACAGAACCATGAAAGACCCTGAATAGCCAAAACAATTATGAAAA
AGCAAAACAAAGTTGGAGGTATTATACTTCCTGATTTAAAAATTATATTACAAAGTTATAGTAATAAAGACAGTATTATACAGCCATAAAAACA
GACACATACACCAGTGGAACAGAATAGAGAGTCCAGAAATAAATCCACGTATATATAGTCAACTAATTTTTGACAAGGGCACCAAGAAGACACA
ATGGGAAAAGAATAGTCTCTTTGATAGGATGTTAAGAAAACTGGATTTCCACATGCAAAAGAATGGAATTGGACTGTTATCGTATACCACATAC
AAAAATCAACTCAAAATTGATAAAAGACCTAAACATAAAAGCAGAAACTATAAAACTCCTACACAGGAACATACGGGAATAGCTCCTCGACATT
GGCGTTGGTAGTCACCTTTTGGATATCACAACAAGAGCTCAGGCCACAAAAGCAAAATTAAATAAATGGGGGCTGGCCGCAGTGGCTCACGCCT
GTAATCCCCGCACTTTTGGGAGGCTGAGGTACGCGGATCACAAGGTCAGGAGATCGACACCATCCTGGCCAACATGGTGAAACCCCATCTCTAC
TAAAAATACAAAAATTAGCTGGGTGTGGTGGCGCACACCTGTAATTCCAGCTACTTGGGACGCTGAGGCAGGAGAATCGCTTGAACCCAGCAGG
CCGAGATTGTAGTGAGCCGAGATGGCGCCACTGCACTCTAGCCTGGAGGACAGAGTGAGACTCCATCTCAAAAATAAAATAAATAAATGGCACT
ACACCAAACTAGAAAGTTTCTGTACAGTAAACGAAACAATCAACAAAGTGAAGTAGCAGCCTACAGATTGGGAAAAAATTCCAATCAGTATATC
CCAATATTCCAATGTACATCAGATATATTGTAAATCATGTATTTGGTAAGGAGTTAGTAGCCAAAATTTATCAGGAACTCATACAACTCAATAG
CAAAGAACAAACGAATTACCTGATTTATAAAATCGGCAAAAGGCCTGAGTACACATTTTTCTAAAGAAGACACAAAGATGGCCACAGGCATATC
AAAGGCTACACAACATCATATCAGGCAAATCCAAATCAAAACCACTGTGATTTACCTCCTCACAACTGTTAGGATGGCTGTTATCAAAAAGTCA
AAAGATAACAAATGTTGGTGAGGATGTGGAGGACTCTTGTCCACTGTTGGTGGGAATGTAGATTGGGTATAGCCGTTATGGAAAACGTTATAGA
GGTTTCTAAAGAAATTAAAAACAGAACTACCATATATCACCCAGCCATTCCTTTTCTGGGCATATACCCAAAGGAAATGAAATCACCACCTCAT
AAAGATGTCTGCACTCCCATGTTCACTGCAGCATGATTCACAATAACTAAGATATGGAAACAACCTAAGTGTCTGATGATGGACAAATAGAGAA
AGAAGTGGCTGTAAGTATACCACATTTGGTTTATACAAAATGAATATTATTCAGCCCTAGAAAATAATGAGAGCTTGTCATTTGCCACAACATG
GGAAAGCCCATTGTGCTAACCCAAATACGCCAAACAAAGACATATTATATGATCTTACTTATATGTCCAACCAAAAAAATTTCAAATATGCAGA
GAGAATACAACAGTGGTTACCAGGGATATGGGTTGTGCGCAGGAAATGGAGAGATCTACGTCAGAGGATACAAAGTAGTATGAACAAGTATAGA
CATCTAATCTGCAGTATGAAGACTATAGGTATGGCATTCATGCTAAGTGAGATTTTAGCTCCTCTTAACATAAAAACAAAAATAGGGTAACTGA
GATGATGGATATGTTAATTTGCTTCACTACACTAACCTTTTTACTATCTATATTAAGCCTATAACATCACGTTGTATACCATAAATATACGTAA
TAAAAATTATTTTTCAAAATAAAAAAATACAAGATAACTTCTCAACATTTCACTGTTCTAACACAAGTTTCTTCCTTATTCACATGAGGTCTTA
AATGGCTGTTGATGAATGGTAGGTGACATTTTCCTCCTGGAAATCCTTCAGGAATTCAGATTCCTTCCATCTTGTAGTTTCTCCATCTTCACCA
TGTGACTCCCAGAGTTACTGTGCTTGTTTGCATCACATCAAGACAGCGGGAAAAACATGGCAGGTTACACATGGAAGGTTTTTATGGAGTAGGC
CTAGAAATTAGCATAGAAATTAGGCCTAGAAATTAGCATAATCACTTTCACTTATATTTCGTGGTAGAACTCAATCACATGTCACCAACTAGCT
GCAAACCACGCTCGAAAACGTGGCCCAGCTGTGCACCAGGAAGAAGACACTATAAGTTTCATGGAATAGTCCCTGTGGGAAGTGGGAGGACACA
GCTAATGCTATGAGGCCTGGATTTATCACACTGTACGTTTTATGTGAGATTTTTTTTTTCCCCAGGAGCTTCAACATTGAGTTCACTGGTGAAG
TTGCTGAGAAAGCAGGTGGTTGAGTTTGAAATTCAGAATTTGGAGGTTCAAAACTGAATAGTTTTAAAAGGAAAACTTGGGCAAGTCTCCGTTG
GAAAATGTCTTTTCAGTGTTTCCGTACATTTTCGCGTTGATATGAGAAGGGCATACTCACAACATATGTCACTCTGTGTCTTCCTGGTTTTCGG
TCCTGCACCTGGACAACCAAACAGGACTCGCCCCTTCGTTCCTGCTTCACGTTGGACCCTTCAGTCGGAGATAGTATGACTTACAGAGGGAGAG
ATGTTTTCAAACCCTGTGAAACCACCCATTGTTCAGATGAACCATTTATCTTGCACTTTTCTCTAATACAAATGAATGAACCTTCCTGCTTCTA
GCTAGTACCCTTATTCACCTGCTCTTCTGGTGGTATGCAATGCTCATTACATTTTTTGCTTTTGCAAATTTGTCTCATTGTGTTTCCTTTGCTT
AAAGTTGGCTGTTTTCTTCTTCCCTCTTCTCTTCCCTTAAATCATTGTCAATCCAAATCCTGGACAACCTGTCCTCTCAGGCCCAACTCAAATG
CCATCTCCTCTGTCATTCTCCCCATTTACCACAGTTGAGTCCCTCCTGTCTTCCTGACACTCTTTGGGTCTCAGTTTCACCACTCAGGTGGCCC
ATGGCTGCCTTGATACTTCTGTATGAGTCTTTCACCTACTACATTTAAAACTTGATGATAGAACTGTGACTTCCTACTACTTACTTGCTCTTAG
CACAATGTCTTACACACAGTACATTTTTAGTAAACAATTGTTGAGTCAAAATCAATAAATATTGCTTATTGTGGACCACATAGAATCAAGTTCA
CTTTGGAGAATCAGTCTTGTGTGGCCACAGATCACATCAGCAATGTAAAAATTAAATAAATAAAACAAGCCTAAGATAATGTGTTACAAGGGGA
CAAAGGCCTCTACCGATTTTTTCCATCTCTAATGATCATAGAGATATGATTTGTATTATTTATGTGGGCTAGAAAAGGCGATCTCTATTAGGGC
TCTGAAGTTTGTGAAGGCTCCAGCCACCCATCTGATGTTGAAAAAGTCAATTCATTGACAACCCACTGTATTTACAATCTTGACTTATTTTCCA
GCGTTAATGGAGGCAAACTGAGATTTCGGTTGGTTGGTTGTAGGGAGTATAAGTTATATCAATGCTCAGGGGAGTAGTTTTGAGTTTGAAACAT
TACAATAACTTCTTCAGGCTTTTAAGATGTACATAAAATACTTCTAAGTCGATGATAATGAATTGACAAGAGACCTCTTTATACAGCTACAATA
TATCAAGTTGTTACTTTTCTACAAATCTCCTTATAGATTGGGTTTTGCTGGTACTGTTTTTATGGTTGTAGTTGTTTATTGTAGGATAAAAGTC
CAAGAGGAAATGTCTCTTAAATATAAAATGTATATTCTAAATATAATATATAGATACTATGTATAAGCTATTTTTCTGCTCTAGGTCCTTTTTA
TTTTCTTAAAGCCAAACATATTGTGTGTGATTCAAACAGGTAGAGAGCAATTAATTTAAAAGTGGCCTAAAACCCACCTTCCTCTACTACCTTT
GAGGGGCATTATACCATTATATGATCCAGGATTTATATAGCAGATTATTTTATCACTACTCCCTTAGAGCAACCAATCAATCAGCTTCATTTTA
TTAGGATGCTTTAATATACCCATTTTAAAAATGTTTATTTAATATTTTTAAGTAATTTTTGAAAAGATCTAGGAAGATTCACATACTCATTTTT
TCATGAACAAATTATGCTGTATACTTAATTGAGTCTGTCTGTACGGATCATTTATTTCTATTCATCAACTCTTTACCAACCTTCATACTAGATA
TTGAGATGATAGTCTATTGATTTTGTTTCTCTCTCTCATGTTTTTCTAGTATGTCTTATCTTTTGGGGTTTATTTCTGGCCCTGGAGATCATTC
TTTTATGTCAGTTCTCAGCTAGTTGTCATTTGTTGCTAAATTACCTGCATTCAAGTTTTTATAAATGAATGTAGATATTTTGAAAACTATTTCT
TTTAGGATACTAAACAGTAAGACAAAGATACACTGCACAATTGTGTACAAAAGGTCTTTTGGAATATGACTTCTTTGTTAAAAGCAATTTGTGC
CATGATAGTTTAAAAAAAATAGTTCCGCAATCATGTTTTCATTTATGACTATCTACATACTCCATTTGACCAGAGAGAAGTTTTTTATTATCAC
TAAGGCCTCTGTGTAACCAACTTCACCTCAAATATGAAATATATGGTGTAACAAGTGCTGTGTTTTCTCTTTAAAATACACTTGGATTCTTCCA
CGAGGTATATTAAAAATGCTTTGACATCATTATTTCATCTTGAGTTACCCTTTCATTCACTAGCTTGGTTGGAGAAAATTAACTCTTCAATACA
TGATACAGAAAATCTGTTGATGCAGCCTCCCTCCAGTTCTTAGTATTTAATTTCATGAGGGACATAGCACTTTTCCCCATGTAATTACAGATTG
GTAGAGTTGAGCAAGACCTTAAACATCCTGAAGTTTAACACCCATCAAATGCAGATACCTCTTCTAAGGTATCCTAATTTCTATATCGAAAATA
ATTCTAATTATCAGTTTACTTGGCAAATCTGCCATAATAAAGTCTTTTTCAAAGTTATCTATGCCCAGTTCCCCTCAATATTTCTCATAATATT
AGTTCCACAGTCATTCTGATCTCACTCCTTTTTGCTTTCTCCAGTTTATCAGAAACTTCCTTAAAAGGTAGTGGACAAATTATAGAAGTACCTC
CCTTTTTTTTTTGAGGCAAGATTTCACTCCTGTCATCCAGGTTGGAGTACAATGGCGCAATCTTGGCTCACTGCACCCTTCACCTCCCAGGCTC
AAGCAATTCTCCTCCCTCACCCTCCCAAGTAGCTGGGTCTACAGTTGCATACCACCATGCCTAGCTAATTTGTGTATGTCTTGTAGAGACGAGT
TTTCCCATGTTACCCAAGCTGGTCTTGAACTCCTTACCTCAAGTGGTCCGCCTGCTTTCCCCTCCCAAACTGCTGGGATTATAGGCGTGAGCCA
CTGCACCTGGCCCTAAAGCTACTCTTTCTGAGCAGTTTGAGAGGGACTGTGGCTTATCTCGCCTTGAGAACAGTGTCTTTATTAATGCAGCCTA
AGAGTACATTTTTCCAAACCCATCACACTGCCTGTATTGAATCAAGAGGCTGCTCCACCTCCATGTACTGACCATACAGGACCTCACATTTCAG
CCTAGTAAACTTGACTATATTCATTTTGGTCTATTTTTAACCTTGTGAGAACTTTTGTAATCCTGTTTCTGGCATCCCTGGTGTTGTGTTTTTG
TACCAATACAGCTTCTGGATCATTGTCTCAATCATTGATAAAAATGGGCAAATACACACTTTCTGTCCACAGTGATAATCATATCCATTACTTT
TGCTTTAGATAAGAGTCTTCCTGTTGATCATCTTTGCCTGGGAAGTTCAATGATAAAGCCAGAAGAAAGCATCCTTTCTGATCATTGTAAGTTT
TACTTTTAAGGAAAGAGACTAAAAAAATTTAGCAGGAAATTTAAATTTTATTAATATCATCTCATATACTTTAGAAGATCTAAAACTGTTAAAA
GAAACAAAATGGTTTACACTGGAGCCTGGTTATAACCTTTCTTTGGTAGCCCATAGCTGTGGGGCCCATAGCTTTGGGGCCCAAAGCTCTCTTT
GATTTGAGTCAGACACCTTCCCATGTGCCCAGGAATTTTGATTATGGCAGCTCATATTATCAAGTCTTCATCAGATTTGGCTGGAGGGAAAAGC
TGTCTGTCACCAAGTTGTGTGAATCTTTGTATTGCTAAAAAGGATACCCGGGCAGTCTTAAAACTCGTGCCTTATGTTATTTGAACTTTAAAAT
ATTCTCATGTTCATGTTTGCTCAGGCTGGCTACTCCAGTTTTGTTACAACATAGTGCTGCTCAGACCATCGTTGAAAGTTTGATGCTAAAAAGC
ACAGTGTATTTGAGCATAGTCAATTCTAAAAAAAATTTTGTTTTCTTTCTTCCTTCCTTCTCCTTTCCTTTCTTCTTTCTGTCTTGAAATCTGT
GGATTGAACTTTCCCACCCCTCCAAAAACAACTCAGAAAGTATATCATGTGTCATCACTATCATCTTCCTTTTATCAGTGCAGGTTTATAGAAA
GTGTTCTCATATCATCCTGAATCATTTCTACAAACTATAAAACAAAATTAACTTTCCATTACAATCCTAACAATTGTAATAAATGCTCCTATTA
CAAGCTTTTACTTCCATCCTGTGAAGTATATCATGCTGAGGCGAAAGTTGCATCTTCATTAGTCACCCATGACTTACTCCAGGTGTTACCCTCT
AAGTTAGAAAGAGAGAGGGCAAAATACCCTGGTGACACATACGTTTTTTAAGGGAAAAAATTCTGAAAGCAAAAGCGTTCTCTCTTTTTTTAAT
ATGACTAGCAAAAGAGTGTTGATTAACATGTGTCAAGCAGGTGCTGGGTATGTGCTAAACTAATGGTTTTCAGAGTACTCAGTGGCTATTTTTA
AATGTTTCTATATCATTCTCAGTTAAGCTGCTGGATGAAAAGTACAAAAGGCTAAAAATATTTACAGTTTCAGTAATGTCTGTTTTAAAAGAAA
CCATCCGCGTCTATGGCCACACCACCCTGAACGTGCCTGATCTTGTCTAAAAGAAACCATCCTGTGTGATTTTTCTTTATTTCCATTTGAAGGA
AAAGAATATTATGGCTTACAGAGAAGTGGTTGAAAGCAGTTTGTTCAACACTGAGGTTTTTACGAAAAAATGGCCATCAAAAGGACAGTAAGTT
CGGTTACAAATCATCACGATTAAAAAAAAAAAGTTCATTATTATTCCTTGTACTATATTATTATTACTATTTTTGTATTATTAACTCTAGTTCC
AACTGAAAATAGTATTATCTTTGAAACTTTTTGTATGGAGTTAGGAGACAAGTTGCTGGTATCAGATTACTTGGGCCTATATCTCAGCTCTATC
CCTTGGATATGTGATCCTGGATAAATTGTTTAACCTCTTTGTGCCTCAGTGTACCCATCTGTAAAACAGAATAATAATAGTGCCTTCCTGCTAA
GGTTGTTGTGATTAAATGACATCATGCTCTGCCTAACATGGTAAGTACTCAGTAAATGTTTGTTATGCTTATTTTTTTCATTTAAAAAAATTTT
TGGTTAAATGAACACATTTATTCTCTTAACCATTTTTGAGTATATAACTCAGTAATATTAGCTATATTTACATTGTAGTGTAACAAATCTTTAG
AATTTTTCATCTGGCAAAACTGAAATGTTTATTATGTTTTAAAACCTCATTTTTCTTCAGAGTCAGTTACACAGCTGTGTGGACGTAACACACG
AATCCAACCACCATTGGGGCAGGGAGTTTAGACTCTTCTGTTCACTGCTGTATCACAAGTGCCACAATCAGCATCTGGCCCATAACAGGGCTCA
ATCAATATCTGTCGAATGAATGAAGTAATGAATGAATGAACAGTGAGGCTTCATTTGACAAACTACATGTGTGTTTGAAAATTATTTCTTAAAT
GCTAAATCCATTTTTATGAGTGATTTTAAGTATACATCCAAAGTTGACTCCTTTCGGAGCGTCAAGTGATTTCTAAAACAACTGTTTTCTAATT
ATTTAGCAGTTCTGGCTTTGGTAGGTCAACTTTTCTTAAACTGGAAAAAAAAATAGTATTCTTAGTGTAGATTGCAAAAGGAAAGAGGATATGT
TAGATGATTTGCCCAAAATTTAAACAGGATTCGCTTTTTAAATTCAAGCCTTTTTGCTTTCATTCAGATCTGGTCACCAGACTTACTTTTTCCT
TAAGGCATATATGAATCATCTTGTTCTTTCATTCAGAAATCAAAAGTTCACGCCTACATCAGTATTTACCAAAACTGCTGCAACACTACCTTTC
CACTGAGAGTGCATTACCTTTTTTTGAGTCTTTGAAAACAATGTCAGCTGAACATGGTCTGGAAGTGGCCAAAACTCTGTGGGTGGGGAGAAAG
GAAGAAATAACCTATCTCCCATTTAAAAACCTACCACCTATTAAATAAACGGCAGTCTTGTTCTGTGGAGTATTCATTTGGGTTATCTATTCTG
ATGAACAAATACAAATTCTTCGTAGGGTTTTCTTGCTTCTGGATAAAATGCTTAGTGTTGCTTTTTGTTCTTCAATCTTAGGTTTTGTTTCATT
TTGTTTTTCAGGGTCTCACTCTGTCAACCAGGCTAGAGTGCAGTAGCGCAATCATGGTTCGTGGCAGCCTCAGCAGCTCCCTGGGCTCAGGTGA
TTCCCCCACGTCAGCCTCTCCAGTAGTTGAGACTATAGGCACGCACCACCACACCCTGCTAATTTTTGTGTTTGTAGTAGACATCGGAGTCTCA
CCATGTTCCTCAGGCTGGTCTCGAACTCCTGGACTCGAGTGATTTGCCCACTTTGGCCTCCCAAAGTGCTGGCATTACAGGCATGAGCCACTGC
GCCTCGCCTGTTTTTATGATATGTATTGAAATGCTCCAATAGCATTTTATTTTTATTTAAACATTGCTGTTAGCTACTTGGCACAAAATGTGTC
TTATGCCAAAGTTTACTTCCTTCTACCAAGGAAAGGGGACTCTGTCTCCTGGACTTTGCAGTTATGGGAGGAACATGACAGGAAGCTACATCAT
CTGCCAGGATTTTTATGGGGCCACATCACATACAGTGCCTGTCTGTAACTAAGAGCAAGAGCACATCTCTCCTTTCAGCTGATGTTTAGCAAAC
CCTCACTCTGCTCCCATGGCAAAGACAGTCACTCATCTTCCAAATTGTGGTATCTTGAGTCATTTCCATTATGACTTGTCTTCTGAAAAGTGAT
AAAGCAAAATCATCTTACCTAGTCATTTTTTCCCATTCTACAAGACAAAGTTTATCTCTGAATCAAAGAGCATAAATCTCTGTTGCCTCTCTGA
GATAATAAAATATTTTTATATTTAAATTTTCTGTTAACAGGAAAATGTATAAAACCTTCCCAAATTTTTAACTCCTTCTTTTGATTCTATACCT
ACATAGGTTTTTATTTGGATTTGAAACCAAATAAAAGACCGTTTCATGGTCTGGGCCCTTGGGACAGCGTAGTAGTGAAGCAGCCAGTTCATTT
ATTCTTCATCTCCCCTGCCATGGCTTCCTGTTGCACTAAGAATCCAGCCTCCTCCCCGGAGTTGGTGGGAGTCTGCTGACCTCGCCCCAGCCAG
GCTCTCTGGCCTCATGCAGCCATTTTCCCTTCATTGACCCCATACCACCACTGTGACCAGGTGCACCAGGCTCTTCCTGAGGCTGCTTTTCTCC
CTCAAATCCCCACCCCACCTCCCACTTTTCATTTTTCTAACTCTCGTTCATCCTTCACTTGCATTTTAACATCACATCCTCTGAGAAGCCTTCC
CTGATCTCCACCCTTAATGAGCACCACCCAGTTGTCCATACTTTGCTTTCTCCTTTCTTTCCCAGCCCTTATTAGAATTTTAATTTCATATTTG
GCCATACATTTGTTTCTTTAATAGTTTTCTCTCCCATTAGTCTATGAGCCCATTAGCGATCACGGCTATTTTGTCTGCTGTATGTAATATCCAC
TTCCTAGCAAGCAGGACACACTCAAGGAAGAGTTATTGAATAATAATTTAAAAATATTTTTTATTAAATGAAAAATATAAATATCCAATTCTAA
GAATGTGCAATTCTGTCAGGAGTGTTAAGTACTGAAAAAACGACAGCAGATTAGTTAATTGGTTGATTTCACAAACATCTGTAGATTGTTGACA
GTGCACTATGATGTCCTGTTCTAGCTGCTGAGTACATGTGGACGGCTTGTTGCCTGGCTTTGCAGAGGTCTCACCTTGGGCAGATAGCAAAGAG
ACACAATGAGGAATAAAATTAGTGCCTTTCTCTCAAGGAGCATATGTATTCATTCATGTAACAAACATTTGTTGAGCACCTATGATATACTCTA
AGCTAGAAGACACTTGAATGTGATGAGTATAACATATAAGAGAGTAAAATTAATCTCCAACAAACATACAGAATCCTCTACGGATCAAGATTAA
GAAATGCTGTCTGATTAGGGACATCAGAGAGCATCCTGGATAAAGCAGAGACCGAAGTGCTACTTGAAGAATGGGTTCAACTTTGACAGCTCAA
GCTATGAGGGAGGAAATTCTAGGCATGGGAACCACTTGGGTAAAGGCATGGAGATGGGAAGACATTCCAGGGATACCTATTAACCCTTTTTAAC
TGAAGCATAGCGTGGATGATGGTGAATAAGAAGAAATTAGACTAAAAAAGTAGGTAGAGGCTATACTTTAGCGGAGTCATGAATTCCTCTAAGT
TTAGAAAAAGTTGATTTTAATTACCTTAAATAAGGCAGTAATGGTACTAATATTCCTGTTTGGTAGCCCTTAGGAAAATGATACTTGTGACTTG
GTACAGTGGCTCACACCTGCAATTCCAGCACTTTGGGAGATTGAGCTGCGACGATTGCTTCAGCCCACGAGTTCAAGACCAGCCTGGGCAACAT
AGCAAGACCCCATCTCAAAAAAAAAAATAACTGTACATGGTGGTGCATGCCTGTAGTCCCAGCTACTAAGGAGGTTGAAACAGGAGGATCACTT
GAGCCCAGGAGTTAGAGATTGCAATGAGCTATGATGACCACTGCACTCCAGCCTGGGTAAGACACCATCTCTACAAAAAAAAAAAAAAGAAGAA
CAAGAAAGAAATTGATACTTGTTTAAATATATTACATGTCAAATGTACTACAACAGTCCTCTCCAAATCAGAATATAAAGAGCTTCATCCTTGT
AAACTGCAGTCATGTTCTCGTTATAGTGGTAGTTAGTATGCAGGTCAATAATTTATATGTGGTCTTTAGCTTTGTCTTTTTCATTAAACAGTAA
AAACGGTAACCTGACATTTGAAGGTTTTTTTTTCACATTTGTAATATCAAAAACTTGATTCGTAACAAGTATCGTAAATTTACAAGTATGTTTT
TCATGCATATTTTAAGAAAATTTTCATAGGACTTTAACGTCAGGTTTCTTTTGAATACTTCCACATGCTCTACCTAACAATTTTAGTGTAAAGT
TTCATTTTCCTTATCAAATTCTATGTAAGTGAGACATTGGTGTTTTAGCCAAGGTACCCTGCTTTATGGAGTATCTGTGTTCCTAGAAAATTTG
TTTTCAAATATCATTTTGTAAATCAGATTGTATTGTAATATACTTGGTGACCTTTTGCTTCAAATCACCTTAAAATAAATTCATTTATATTATC
TAATTCATATTTTATAAATCCTAATTTTTATATCTACTCATTTTTCTTGAATTGGGGGTAATTCTACAAACAATAAACAAGGTAAATGATTTTT
TTTTGCCAGTTTGTACTAAGAGAACATGTTATTTTGTTTCGGGTGAATGTATGTATCTTAATACACTGTACTACATTAGACATCAATAGGGAGG
AATGTAACGCATTTTTCTTTTGTATGGAAATCTGTTGAAACCTATCTTAAAGAGTTTTGAGCAGAAATTGAAGGATGAGCCTGACGTTTTCTCA
TTAGTTGGAGAATATTGTTTTCACTAGACAACAGTAAGAATAAAAGTGGAAAAATTGTATTTAAAATTGATGTTTTATAACTTTCAGAAGTTCA
TCTCTCCATTTTACACATTATACTTATAAAAGTACAGTAATCAACTCTGCTTTATAACTGTTTTGTGAAGATTATTTAAAGGTCAAGGGAGTGT
AGTAGAGCCACAGAAAAACACAATAAAATTGGAATCCTAGAGTTTATGTGCATTTACGTTAATTTTGACTTAGTGGCTCCTGGCTCTCACCAAT
CTGGGTTGAGTGAAGGACCCACTGTTCATTGTGTTCCAGCAGTAGTCACTCAGACCCAGCTTGCCACGTTATCAGTCCAACTTAACTTCTGCCA
CGTCCCACTCCTCCTCCCTTGATAGCTTCTCTTGCTCACATCCTTTCCACCCCTTTGTGCTGCATTCCTCTGGCACTGCCACCCTCCTGTCATG
GAATCAGCGTCTGGCCCACGAGACCTTTGGAGAAAATTCCTGGAGAGCAAGTTTTCATAGATTTTTCTCCAATTTTTTCTGTTTCCACCCTGTT
ACAAATATTTTAATCCAAATATGAAAAATCTTTAAATTCAGACTTCTCTTGTTTCAAATTCAGACTTCTCTTGTTTCAAATTCAGATTATTTCT
GATCACCTCATAGCTAGTTCTTGAAGTXCATCTTAGCAGGAACAGAGCTAACTAAACTACTGAATGATGTAAATGAACATATAAAGGCTATGTT
TACCTACTTAAGAAATTGCTTTTAGCCAGTTTAGAAATGTCTCCTTACTCTTTTGTTTTTAAATTTAAAAAATTCTTCAGAGATGGCATCTCGT
TCTGTCAACCAGGCCGGAGTGCAGTGCCACCGTCACAGTTCACTGCCTACCTCAGCCTCTGGAGTAGCTGGGACTACAGGCACCCACCACAACA
CCTGGATAATTTTTTTAAATTTTCTTGTTAGAAACAGAGTCTCTCTATGTTGCCCTGACTGGTCTTGAACTCCTGGCCTCAAGTGATCCTCCTG
CCTTGGCCTCCAAAAGTACTGTGATTACAAGTGTGAGCCACTGAGCCTGGTTGAAATGTCCACTTACTTTTAAAAATGAATTGTATTTTAAGGA
AGAGTTTGCAATGCTTAAATAATTTTCATCAGTTTACTTAAAAAGTACTAAAGCCTTTTTTTTTTTTTAAGACAGAATCTCACTCTGTCACCCA
GGCAGGATTGCAGTGGCATGATCTCAGCTGACTGTAACCTCTGCCTCCCAGGTTCAAGCGATATTCTCCCACTTCACCCTCCCAAGTAGCTGGG
ATTACAGGCACCTGCCACCACACCCGGCTAATTTTTTGTGTGTTTTTAGTAGAGACAGGGTTTCACCATGTTGGCCAAGCTGGTCTCGAACTCC
TGACCTCAGGTGATCTGCCCACCTTGGCCTCCCAAACTCCTCCGATTACAGGCGTGAGCCACCACACCTGCCTAAACCCTATTTTTAAAGCATA
ATTTATATTACGTATTCTTAATCCTTCACGGTTCTTGTTTGGTTAATTTCAGCAGCTTAAGGAAGATCACTTGGAGCTAGAAACGCCCATGAGG
GTGATGATGGCTCAGAGGGGGCAATAGAAGCAGCTTTTAGGGAGCAGTCAAGTGCAGTAGGTAAACATGGGATGTGTCAGATATGATACCAGCC
AGTGGTCCCCATAGCACAGGCAGCAAGTGTGAGGCACAGCACAGAGCACATGTACACGAGTATTTGTGTGCGTGTGTGCGCATGTGTGCCCGTG
TGAAGGGGGGAGGGGGTGGTTGCTGTCAGGTGAGCAATATACCCAAATGGAGACCCCAGAAGCAGCTGATACTAATCCATCCAAGTAGGCAGCA
GTGTCTGTCCATGAGAATGTTGGGCCCAGCCATTATCCAAGGATTCTAGACCAGATTCTGGAGAGATCACAGCAGAGTTTCTGCTTGACTTCAC
TCAGCTTCAGTGTTTGAATCTGTAAAATGGAAGCAGTAATAGTAATTACCTCAAGGGCTTATTTTGATCGCTAATTGAAAACATGTATGCGAAG
CCCTTGAATACCATCTGGTACATAGTAAGTATTTAGTAAATGTTACCTATTACTGTATTACATGAAATTGGGAAGACAGTATTTCTTTTTAAAT
GTCCTATAAATAACCGGGTGCAGTGGCTCATGCCTGTAATCCCACCACTTTGGGAGGCCCAGGCAGGCGGATTGCTTCAGCTCAGGAGTTCAAG
ATCAGCCTGGCCAACATGGTGAAACCCTGTCCCTACTAAAATTGCAAAAATTAGCCGGGTGTGGTGGCACATGCCTGTTATCCCAGCTACTCAC
GAGGCTGAGGCATCAGAATCATTTCAACCCAGGAGATGGAGGTTGCAGTGACCTCAGATCATGCCACTGCACTCCAGCCTGGGCAACAGACCCA
GACTCTGTCTCCCAAAAATAAAAAATAAAAAATGAATGTCCTATCACTGAGGTTTATTGCTCCTCAATTGAGCTATTCCAATCAATGAAATTTT
ACTACATAAAGGTAGATGCCTTCTAATGGTTTTCTTCCCACAGGGATGGTGGGGTGAGATGGGCAACTGTTCAGGTAGTAGACCTGTTCATGAG
GAAAAAAACTATTTAAATGCTCACAAAAGGATGCTAGCTTTTTTGGATCCATCATCATTTTTGCCAAATTCATAGAATTTTTGCCCAGTGGTGT
CAAACTTTAAAATTCATAGGAAAAAGAGATTCAGTAATTACTCTGTAATATTAGGGAGGGTGTGGAAATAACGAATGAACGTTATGTAAAGACC
ATAATGCTAGTCAGAGAATGTTCCAATGGAAAACTGATGTTTCCTTCTGCCACCAGGACAAAACTTTTGTGATCTAAAGGCAAAACAATAACAA
AAACAGCAACAGCAACAAAAACCAGCCTAACAGTCCCAAGTGGTCTTCACTGTCATGATTATTAAGCATTTTCTCCAAAGTCTGAACTACAGTT
TGTTGAGGTTTCATTGAAAATGATACTAAAACAAATTTGCTTAAAAGTAAAATGGAGTTATTGGCATCACAAAGATCATAGTCACCTTCTTAAG
AATTCCATAACCTTTAATGACTGTAAAGGATTTTTAAAGACGATCCTTGCCACTAGTTATTAATACATTTTTTTGAGGCTGTGCAAATCAGATA
ATTTGGGCCGGGCACATTGGCTCACGCCTGTAATCCCAGCATTTTGGGAGGCTGAGGCGGGCGGAGCACCTGAAGTTGGGAGTTCAAGACCAGC
CTGACCAACATGGAGAAACCTTGTCTCTACTAAAAAAAAAAAAAAAAAAAAAAAAAAATTAGCCGGGCATGGTGGTGTATCCCTGTAATCCAAG
CCACTCGGGACCCTGAGACACCACAATCGCTTGAACTCGGGAGGCGGAGGTTGCAGTGAGCCAAGATCACCCCATTGCACTCCAGTCTGGGCAA
CAAGAGCTAAACTCCGCCTCAAAAAAAAAAAGAAATCAGATAATTTGCCCAAAGTCAACAGCCCAAAGTCAACAACTCCTGAATTTTTTGATCA
CATTATCTTTCAAGTTTAGTTAGAACCTGAAAGTTAACTGTCCTTCACTTAGGTAGATCATAAATAATGCACTGGTCACATTGTGTAGATATTA
AGTTACACTTTGGTAGATGGATTTTTAGATATTGAAAATGATAGATTGCTACAACTGCTTTGAAAGTTTGGCAGCTTCTTATAAAGCTCAAGAT
AGGCTTAACGTACAATTCAGCAACTACGCATCTAGATGTTTACCCAAATGAGTTGAAAATTTATGTCTGCAGAACAACCTGCATGCGAATCTTG
ATAGCAGCTTTATTTATAACTGTCAAAAACTGGAAGCAACTAAGGGATCCTTCATTACATCAATTGATAAACTGTGGTACATCCAGATAATGGA
ATATTATTCAGCACTAAAAACAAATGAGCAATGAAGCCACAGAAACACACAAAGGAACCTTAAATGCATATTGCTAAGTGAAAGAAGCCAGTCT
GGAAAGGCTACATACTGTACGATTTAAACTATAGAAAAGGCAAAAGTCAAAACCATAGAGACAGTAAAAAGATCAGTGGTTGCTAGTTCAGGGA
AGAGGGGACAGGATGAACAGGTGGAGCACACTGCATTTTAAGGGCTGTCACACTATTCTGTATGATATTGTAATGGTGGATAGATACGTGTGTC
AAAACCCATACAACTGTGTAACATAAAGAGTGAACTCTTATGTAAACTATATTGACTGTTATGACTAGTTAATCCTATATCATCATTGGCTCAT
TAATTATAACAGATCTACCATACTTGTGCAAGATGGTTAATAACAGGGGAAACGGGGAAGAGGGAACTTTTGGCTTAGTTTTTCTATAAACCTA
AAACTGCTCTAAAAAATAGTCTACTAATAAAAAAAAAAAAAGAAAATCAGGATAGATTGTAGCTTAGAAATAAAAAAACAATACTCCAAGACAA
AACAAAACAAAAGCCTTCTATAATCTCTTTCATATAATATTCCCCACCTTATTTCTCTAGTTGTTTTTTAATTTTTTTTTCATTTATCTTATGT
GTTATCTCATCAACCTTTAGTAACGGGCAGTTAAAAAAATAAGCAGTTTGGCAACTTCCTGTGGCATACATTTCAGATTGTGAGCAGAGATCCC
CACCATGTGACTGGCAGGTGTCCCTCTCCCTAGCCACACCCTGTCCCTCTTTTCCACCTACCCTAGCATTAGTTGTCTCCTACCTTGCTTTTGA
CAACAGACAGGGTGAAAATGTGGGCTTGTGGCCATTGTGCTAAATGTTTTATTTCTATGTATTTTTTAATCCTTACAGCAGTTCCCATTTTGTA
GATGAGGAAATGAGAGCACTGGAAGAGAGGAAATTCCTACCCTTGCAGTCTGACTCCATTCTGATCCTCTATCAGGCTAATGTGAACACACCTC
ACTCTCTGTCCCAGCCTGCCCCCTGTTGAGTAGGTGGGAGGGGGAGCCATTTGGAGGATTTGGTCCCAGTCCAGGTGCTATTTAAGTGAGCGGT
TGATTTTAGCTGTGCTGAATACCACCACCCTACAGGCTAGTGTCTGCCATTGAGAAGTATCATCTCATCATTTTCAATCTATTAACTGATACAT
TATCACATTATTATTATACATATATATTATTATATATTATCTGGAAACCATATAACTATAATTTTAGTCAACTCTTTTTTTTTTTTTTTTGAGT
TGGAGTCTCGCTCTCTCACCCAGGCTCCAGTGCAGTGGGGCGATCTCGGCTCACTGCAACCTCTCCCTCCCAAGTTCAAGCGATTCTCCTGCCT
CAGCCTCCCAAGTAGCTGGAGACTACAGGCGCACGCCACCACGCCTGGCTAATTTTTTCTATACTTTTAGTCAACTCTTAATTTCTAGCCTCAT
ATTGACAAGTGATAGGGTTCAAAAGTAATATCTGTTGGCTTTGGAAATAATATTATATTTAGTATTATATTTAATGGAAGTTAGCAGATTTAGC
TTCTTAATAATGTTTGGCTGGAGGCAGTATTAACCTTTGGTTGCTGTCTTGTGGCCTGCAGATGTTTGTAATGCAAGAGATAGGAATTTGGTCC
CTAGGAGGGTAAAGCTAAGGTTCACTCTAAGAAAGCCACAAAGCAGACAGTCTACATGTTGAGGATTAAATGTTGATTTTACCTGGGTCATCAT
TTTTAAAGTCCATTTGCCTTCTTATTTTGTGATGAATATTTAACATTTTCAACTACATCTCCTCTTATTTGCTTTGTAGTTCTTATGAGGAAGA
ATTCCCATTATAGTGGGGCTGAGCACATAACCATTATTTTTCCTTATGCTTGTTTTCTTAATATCGTAAAGCAGACAATAGCAGGATATTTAAT
GAAATACATCATGAATTTAATCCTAGACCAAAGCAAAGTATTTTCAAGTATTTTTCATTTTCACATAAATTATATTAAAATTCATATTTAAGTT
AAAATTTTTGAGACAGTTTACTTGAAACATAGTAGCATTCTTCACTGATGTTAGAATTACTTTATAAGTTATTTTGCATTTCTTAGGAGGCAAA
ATAGGTTTTCAAAAGGATTAACATGTGTTAAACTTTTTGATATTTCATTAAAAAACATAGTATTCGAAAAATGCAGTATTTTTTCATTTACTTC
CAAATGTTTTGTATTAAACTTTTTTTGACCTAGTAGATGACTAGTACAAATAGAACTTTTAAAGATTAGGATATACCTGTTACTTGTAACATGA
ATTTTTTTAATGCTTAATTCAAATAATAAAATATATCTGTTTTTGTATTTTCTGTAACAATATACAAGTAGTTCTCAGATTTCTTTTTCTATAA
GATGAATTTTTAGATCTTTATTTTACTTGGCAAGTCTAAAATAGTGGTCGTTCTGGGCTTCATTCATCTGTTCGTTTTTCCTACTGTTTGGCTA
ATTCTTGTATGTGGTTTTAATTTTTTTAATAGCAAATATATATTTCTGTATCTGAATAGTCAGCCTGTCTGTGTGTGTGTGTGTGTGTGTGTGT
GTGTGTGTGTGTGTGTATTCCCCCCCCCTTACTATGGAGATCATGCATGTCAGTCCTGAGCTTGGGGAAAACAGAAAAACAAACCTGACCTTTG
ATCCCTTCAGAGATAACATAATCTCTTAGTCATATAAAGATAAGGTCCATCTCTTAGTCATATAGACGAGATTTATATCTTAGTCATGTATCCC
TATATACATAAATATGTGCATATTCCCACAGATTATTAAAGCATGTACATGCATACATAAATAATTTTTAAAGTGAAAAGAGAATCCTCCTATA
ACACCTTACAGGGTGGCTATGTTCTTTTAATGTTTCATGGGGACCTTGCTGTAATTTTAGAGCCAGCACCTGGCTCAGGTTCACCCATGACATT
TTCAGCTGATCTTTCGCTTGTTTTCTAGTTGACCTTCAAACACAAAGTTCATAGCCTCTTCTCAGCTGTTAATGTGATTTACTTCTTATGTCTG
GGAAGTCAAGATTGTTGCTGAATTCATTCTTTTAAGCCGATTCAGCTCCTTGGTTTAATGCCAGGACATTCCCAGCTGACAGAAAACTAAATCC
GCCAACCCTAATTTCAAAGATTACCAGGTTTGTGGCTCGTGGACATCTTAGATCCTAGGTCGAGCAACTCTTAGGCTGTTCCTGGCACACCACA
TACATATGGGGTTGTGTATTGATTTTAATTCAAGTATCATTTTAGCATTGATTGGCAAACACCCTAGTGGCTTTCAGTCAGTAAAATTCAGTGC
AGCAGCTAACCTGCTGCTGCTCCTCCCTTAAAATCAGTATTTGAGTTAGTGGTCGTTTAACTTTGCAATTTTGACTCTGTCTTAAAATGTATAC
TTTTTCTTAATTTTCAAAATAGGAAGTGAGGTGTGCTCTTCACGTTCTTGGGCTCACAGCAGGGAGGTTATGAAATATAAATGAAAGGACACAC
TAGATGAGCTCCACACAAGGCAGTATCACAACTCACTCCCCCAAGAAGGAAGCACATCATCATTTCTTACACCGTGAGAAACAGCCATCTTGTC
ACAGGAAAATGAGATTGAAGAATTAGTCTGATTAAAAACAAATTACCACAGTTGCTGTTCAGTTAGGTGGCCTGCAGAATATTAGCAGCTCTGG
TTGGAAAGCCATCAGGAGTTTCTCTCAACCAAGACCAAGCTGCTTTCCTTACATCCCTGTCTTGTGTCCCAGCCTGTTTTATGCCTTCACCACC
CTCACTTTAGCTTTTTCCCAGTAGGAAACAATGGCAAGGTAGAGGAAGAGTTTTTAAATTCATTCTTATTAGGAGTGAGTTTTTTTCTTTGTAG
ATATTAGTAATCTAAAAGTAGAAGTAGTGTAAATATTTTTTACAGCATATAATTCAATTGCACATCACCAGTTGCGCTATCTGTATATGCAAAT
ACTCTAAGAAATTTAATATTTTATCAATAGCCACAACTCCAAAAATGAAATCTAATGACTTGTTTCTATGGTAACCACTATCGAAATATGTCTA
GCCAGTGTGTGTCTTCCCATAATTTTTCTCTAAGAGTTTCAGGGAGTTCATTAAATTTGCTTTCCCAACATAGATAGATGATCGATCGATCGAT
AAGAGAGAACAGATACATTTTTCCCCCTAATGGGGTTATGAGACTATTATAAAATTCTCTTTACCTTCGTATAGAGTATGACTTCATTCTTAGA
CATGAATGTATTTGGCTAAGGGACAAGCCTATTGTTTTATAAATTTAGTTTAAACCAGTAATAAAGCCAGGTTAGTAATAATTTCTAAAACGAG
ATTAATCTTGCTTCATA\ATATTTTCTGGTATTTATATATGGAACTGTTGGATTGCACACACATTGATTTCTACAATGACCCATGTCCTTTTAA
AATTGAAATTCATTTGCTGTTCCTTTGAGACCTTGTTAGATTTCTTTATTGCTCGTCCACCGTTGTCTTTGTTATCTACCAGGGCGTTGAGTGT
CTGTCCACCTTGATGGAGTTCTGACCTGGATTTGATCAACATCCCCTTGTGGCCTTTCTTCCTTGTCAGAGGTTGTGTGCTGCAAATACAATGA
AAGTCACAACTTATTTATTTAGAGATCTTGACATTTCCCAGCAGGTTCATTCAGAAAAGCTGACTACAGTGTTTCCAGAGGTACAGAAGAGATT
CCATTGCATTTTACATTTAAGAGAAAGTAAACTATTGGTTTGGTAAATTTTTCACCAGTCTCAAGGCCTGTCATGTCACAAAACAAACAGATGT
GGTCAGATATTCTAGTGATATAAGTGTCCATTCACCCTGAGTATTAACATTATGGCAGGGTTCATGGAATTCTCTGGTTACATTCAAAGGGAAA
CATTTTGTAAAGTAAAGTGGCAGGAAATAGATACTGAGGAAATAAAACTACACTCGGTCTGACAACCTGGAACTTTCATTTAGCCTACAAAGTT
GACAGACAGGCTAGGATGTCTCTGAACCTGTTAGATATGGTTGGTCATAATAACATTTAATTGTTTGTTGAGGAGGGATGACTATTTAACTTAA
GGGCCCTTCCTCTGTACTTTCAAGCTCTCCCAGATTTCTAAAACAAGAGCACTGCGCTGGGCGCAGTGGCTCACGCCTGTAATCCCAGCACTTT
GGGAGGCCGAGGTGGATGGATCGTGAGGTCAGGAGATCGAGACCATCCTGGCTAACACAGTGAAACCCCATCTCTACTAAAAATACGAAAAATT
AGCCGCGCGTGCTGCCACGCGCCTCTACTCCCACCTACTTGGGAGGCTGAGGCAGGAGAATCGCTTGAACCTGGGAGGTGCAGCTTGCAGTGAG
CTGAGATTGCGCCACTGCACTCCAGCCTGGGCGACACAGAGACACTCTCTCAAAAAAAAAATAAGAACATTGGCCTTCCTGAGTCCTCCAGAAT
GGGTTTGTACAATTTCACAGTGGAATCATGGACATAGTATAACTGCATCTACTTTTCAATTTTAATTTGTAGTCTCAGTTCTTTCAAGAAGTAG
CTGAACAGTGACAGGCACAGTTTTCTATACAGTGAGCTAAAGTAAGATAGCTTCATGGAATCAGGTGGTTTCATATCCTGAAATCCCAGAGACT
CTCTTTTTTGCTCCCTCATCTATTGCCCTGTGTTCAGGCTTTACCATTTCTCTTCTGGACAAACACAGGAAACTCTTTACCTGCCTCCTGCTTT
GCACATATGGCCAGCCCCTTGGTTCATCTTCCATACTGCCAACCAAATGTTCTTTCTAAACGCAAAACTAAACATCATGGCATGACTTTGCTTA
AAATCTTCCGAAGACTTCTCTTCCTTTGCAAGGTGTCTAAATCCCAGAATGTCTCAGACCTATCTATCTTTCCAGTCTACTCTTTCTGTGCCCT
GGGTAAACTGTTCGCTTCCCAAGTGCTTTGTGCTAATTGTGTGAACTCTTTCTGGGTCCCACCATGCAGGCCTGAGGTCCCTCATGGCTCAGGT
TTGTCTCTCGCCTACCATACTGTTTCTAATCCTTAGTTGACGTGCCTTCCTCACTAAACTCTAAAAAGCATTTTCAGCACTGTTTATTGCTTGC
TTTTGCTTAACAAATACTCAGCCCAGCACCTCGCACCTAGGTGGTGCTCAATAAATGTTTCTTATATCATTGAATAAATGAATGAAGGAATGAG
TTAGTGATATCACACAGGAAGCAGTGGTGAAGGAGACACTGCATTTAGTTAGTAAAACAGCCTTGAGCTGCAATTAACAGATTTCAGCAAGAGT
GACGCTGGTTCGTTCCTCCGTCGCCCCCATAAGGGCATAGCCCTGGTGAATAGCCACAGGACCAAAATCAGTTTTATTAGTTCTTTTTGAGACA
TGTGACTTGCAAGAATTGACTTATAGTTAAATTTATGAGAGATTAATTCATAATTGATATGCTCATGTATTCTTGACAATGAAAACAGGAAGGC
CAGGCAGCACAACTGACATGGGTCATTTGCTCAGGCCACATCCTGCTGCTCAGTAAGTTCATTCAGGAGGCCCAATATTAGAACTTACTAAATT
CTGAGTAAATGCATCCTTTCTAATTGTCTCTAAGCGATTGTCCATCAAGCTGGATTACCCAGAAGAAGGATTATTTTCTTTTCCTATTTAAAAA
TGTTTTTAAATTTAAAAAAATTTTTTAGAGACAGGGTCTCACTATATCGCCCAGCCTCAAACTCCTTCCCTGAAAGGATCCTCTTTGCCTCCAC
CTCCCAGGTAGCTGCGAACTACAGCCTTGTGCCACAATCCCTCGCTTTTTTAGTTGAACGCCATTTCTCTATAGATCACTGACTTTTACAACAT
CCTTAACAGTAATCAAAACAGTTCTACCTTTGTAATAATAATGGCTAATTCTTGAATGTTTACTGTGTTGGGCACTGTTCTAAATGCTTTATCT
ACTTTGACATAATCTTCACAACCCTGTGATGTAATTACTATTATTAGCTCTGTTTTTACAGACTGGGAAACCGAGAGCAAAGATGTTAAGTGAT
TTATCTCTGTTCTAACGGGTGCTGCAATTTGAACCCATGCAGATGCACTCAATACCTTTGCTTTTAACCACACACTAATCTTCCTTTCTATGAA
TGCAACCAGCATTTCTTGTGTTTGCTTTGTGTGACTTCGTTTAATGCTCCTAGCAACTTCTGCAAATTAGGTGTTAAATCTCTTTTGCTTGCAG
CCGAAAAAAGGTCTTGGTTTTTTGGTTACAAGCAACGGACTAAAGCAAAAAAGATTTTACCATGTCTTATACCTGAGCTATCTTTAGGTCAGGT
TGCTGACTATGATGATGGGTGCCCCAGTTGGTATGGGGAGGGGCAACTTTTCACAAAGGAAGGGGAGCCAATGATGGACAAACAAAATCACATG
AAGAAATGGAAGTTTAAATGACTTGCCCAAGGTCACCCAGCTAGTGAGTGCCAAAGGTGGGATTTGGCCCTAGGCTGCCTCTAGCACCATCTGG
GTTCACTCTGATTTGAGTGGACCCTTTGCAAACTGTACATCCTGCTGCTGCTGTTCTTTCATCAGCAGGTTATGCTATCATAACCATAACATGT
ATCATTTTCTTTATTCCATTCTGGTTCTAAGAATACACTGTGACCACCTTCTTTGAACCTGGCTGTTTCCCAAATGTAGTGCACATTGTTTTAT
TTAATTCTAAACTATCTTTTAGGATAGTTGTATAAACAGTTTAGTTCATCTAAAATTTTAACTAAAATTTGTACTGCTTTCCACTTCTCAAAGT
GACCTTTTTTTCTGACCTGGCTGCAAATTCTGGAGAAGGCTTGACTGAAGCTCTGTGAGCAGACCAGAGCTTTTTCACGCTAACGTTTATACAA
AACATTGACCTTCATGGAAATTCTGGTTCTTGATCACTTCGGTAACCAAGTAAGTTCATCGGAATGCCCATCTATTCACTCTCAACTCACAGCC
CTACTGTACACCTCCTTCCTCCGGAGCATGCTCTCTGGAAAGTGGCTTATACTTTGCACTTCTGAATCTGTTTGTGAGGAAATGACAGTATTTT
TCCGCACTTGCACCACCGACTGAAGCGCTGATCCTTGATGTGTTTAGTTTTTACCTGTGCTCATCTCTACATCAAAAACAGAGGAAGCCACAGG
AAATTGCTGCCTGTATTTCCACGAAACTTAACTACATCATCGCCTCCCCAATCCCCACAGCCCAGAACACCCTGATGCTGGCCTCAGTCTTGCT
GATGTGGGTGGTGATAAGGGGCCCAGAGTGTTCTCATCCTGTGGCTCTGCCAGTGAAAGCCTAGAAGAGATGAAGCACTCTTCAGACTAACAGG
AAAACCCTGAGTCTGGTTCTATAAGCACTTAGTCTCTGAACATGCCGACAGTGAGGGTGAAGTGAGGGATAATTCTATCCCAAGGCTAGTGCTC
CTCTTTCTTTCTGCTCAGGTGTCCTATCTTGTCCCTGGTCCATAGGCCCGTGGCTGGCATTCTCTCTGATCACAGAAAATTCAAATGATCCTCG
GAAAATTGTCACATGAGCCTGTGGATGGGGTCAGGAGAGATAAAGACCATAGGATTTACACTGGCCACCAGCAGTTCCTCAAAGAATTATAATA
ACATAATTGTAATACCAATCGCTGATATTTATTGAGCATTCACTTTGGTGTGCCAGGCATTTTGTCGAGGACCCTATGAGCATTAATGTAATCC
TCACATAAACCCCAAAAGGGACTCTTTATTATTCCCAATTGTCAGGCGAGGTACCTGAGACTCATAGAGTTTGATACCTTGTCCACGCTCATAT
AGCCATTAAAGGATGCAAAACTTGAACCTAAATCTGTGTCATTCTAAGCCAGCACTATACATAGTCAAGTACGTTTTACTCCAGGGTGGAGTGG
GGCTGGCAGCCTGTGTGCACAGGCAGACGGAAGCAGACAAGATCCATCCAAGGCAGGCTGAAGACTTATACCCAGAGAAGGCTTTGGTGTACAT
CTGCCAAAGTGATATTCTCATGTAGAAGAAAGGATTGGGTGATAGTCCAATAAATACTTTACCACAGCGAACTTATGTGTCTTCTGGGGTCTGG
CCAAAGTCATGAGTGCAAGCTCCCAAATAAAAAAAACGTGGCTTCTCACCTCCTCATGATCCCAGCTTCCTAGCATACCACTCTTCTCTATTTT
AGCTTATCAATTCCAAATGTGATTGAAACAAGTATGAGAAATTGATGTTGCTTCAGCTTCTCTGTGAACTGAAACAAATTCTTGTTGAAACATG
ATATCTCCTGCCTCATGGATCAAAGAGGGATGGTCTGGAAGAGAACCCCCCATAAATATTTGAGGCTTAATGAAGGCTATCTGTTGAATAATAC
ACCTGTATTATTGTAAAAAGTTTTAATTTGGCCTCAGAGATACTCCCCACTAAAGCAGACAACAAAAAAGTGAGAAAATGAGGCTAGGAATTAA
TAAAAGATGTCAAGAAAGACCATTTAAGGACTTCTTAGACACAGGCCATAAATGTGGTTTTGAACTTTTTGGCAGCTATTATGAAGACGGAAAG
AGAAGTGTCATGGGAATCAATTTTCAAAACTTAAAAGTTCTCTTAAAATTCTAGCAGAAAAATAGTTTGCAGCTCTACACAGGTATTGGCTAGC
ATTAGTTTAAAGTGGTTGACATGCAGCCCTACTCCACACCTCTTTATATTCCCTTTGTGTTTAGGATTAGAAAAACTCACATAGTATGATCTAT
CATATGTCTACTTCTGGATTATTTCAAGATTGATAATCTCGTTTGTGTATTATCTTGGTCTTCATTTCTCTTTCCTTCTGGGCCTGTTGTTCAT
CTACTGGACATTTCTTTTCTTATTAACATTTCTGCTAAATTTTAAACAGAGAAAAGAAAAATAAATACACAATTCCAGCTATTTGCAGAGGTGA
TTTCAATGCTTTGTCAGCTAAACCCAATTTCAAGAGAGATCTTTCTACATCAGTCTACTCATAATAGAACTCCTGGCTATCTTGGGTTGAGCCT
GCCTGCACGTGTGCTTAACCTGGGCCACAGATTAAGGCAGGTAACGGAGAAACACCTGCAGACGTACCTTTTGACCCCATTATACCTAAGATGA
ACGTCACTCCTTGTGAATTGATATAGAATGTGAAAAGGGTAGAAACTGCTGATTAAAACAAAGTGTTTTGATTATCTGGATTTCAGTAATAAGT
GTTTTCCTCTCTTCCAGTTATAAAGAGTTGAAAACCTTCCAACGCACTCTTGTATAATTCTGTCCTTAAAAGGTTTAGATAACGGTTGGGCTTC
TCTCTCTCTCTCTCTCTCTCTCTCTCTCTCTCTCTCTCTCAGACCAAATATATCATGTCTATTGAAAGTCTGAAGATCAGTACATTTCCTCCTG
TTTGGAAAATAATAATGCCTAGGTTTCACTAGAATTCTTTTTATCTCCAACGCTCTCTTAAATATTGACTAATTAAATAAAAACTAAGGAAGTC
TAGCTAAATACCCTCACTTTGAGGACCAGCCCAGTGGACTTAGAATACACAGTGATTTGCTGTATCTCTCAAACAAGCAACTAGAAATCTTTTA
TTATTGGCTATAATAGCAAGTGGCATCTCTTATTATGTTTGGCAGCTGACATTTCAAATTGCATTTTGGTTGGTTGTTATACATGAAATAGTAG
GATATGAGTAGACTAAAAATGAGAATTTGCAGCCCAGGGATGACATGAAATATCTAACATCTAGGGGTTGCCATGACAACTCATCAGAAAGTTG
CTTTGTAATCTTTTGGATTAGGCTCTGGCCCAGGGTCACTGGATTGGAGTTGGAGAACGCTTTGGTAACAGGATGTCAATGTTGTACCTTTACT
CAATGGCATTTGAATTTGCACTTTGGTCATGTTCCTAAGGTCAAGTAGAAATGACATTGAGGGAATTTTTAAATTCCCGCAGAACCTAGACAAG
GTCACATTTGTCAGCAAAAATACTCAGATGGAATAGTCTGAGACTTACCAACTCAAAGACACCATTTCCTGTCCTTGATAGATAAAAACTATGC
ACATATCCATTTATATATGCCTATCTCTAAACAGTTGTATCTAATGAAAATTTGGGAAATTAAACCCTTTTCATAATTGTCTTCTGTTATAAAA
TTGGAACTGAACACCCCTAAGAAAACAGTTTGGCCACAAACAAACCACAGCTGCTTTCTAGTGCTCTGTGTCCTCCCTTTCAGCTGGAAATGGC
CTGCAGGGTCTGGAATTTGGACCAGGGAAGGGAATTCGGCAGAGAGAGGAAGGAAGCAATTCCGAAACAGGCTACCACATTCCTCATCCCCCAC
ATTGGCCTTGACCACAAGGACAGCATCTTCCACATGATAATGCACCTCTCAGGCATGCTTCTCACCAGGGATGATCAATGGAAGCAACCTGGAA
ACTAAAGAGCTCTGTTCCTAAGTCTCATCCCACTTCTGCCACTCACACTCTGATCTTGAGCAAAACACATTTCAACTCTGGGTTCCCCTAGGAA
ATGAGGGGGCTGAGTTTCCTCTGGCTGTCAAGTTCTGCTATTTATCATCATGTGGGGGCAGAGCCAGGTCTCCACATCCCTTTTCTTTGCCATT
AAGCCCACATTCCCATCATAACTTTGCAGTAGAAGTACCGTTCATTGTTAGTGGGACCTCTTAATAATTAAAAGAAAGACAAAAAATGATTTAT
TCTTATCCCCATACCTTTTAAAGTTGTGGTTTTCTCAAGCAAAGGCTGCCATGCAGTGAGGAAAGGATGAAATTCCAGACAGAAGACACTCACT
TGCAATAAGTTGTCCTACTACCAGTACTGTCAGCATCAGCTCAGCTTATATTTACTGACTATTTTCTATATGCAGACAGTGCTAACCCCTTTAT
ATCCATCAAACTCTTTAATCCTTAGGACATATTCACATGTACACATGTATAGGTATGATGCATGTGATTAAAATTGAACATTTGCTATGTACCA
GGTGTTATGCCAAATCCGTACAATAATCCACTGAGATCAATAAATGGATTCAGTCAGTCAAGCCTCACAACAAAGGAGGTGTTATCAGTTATCT
CCGTTTTTACAAATAAGGAAACTGAGCCATAGCTAGTAACTAGTGGAGCAGGGATTTGAACCCACGTAGTATGGCCTCAGCATCCTTTCTCAAA
ACCTCTCTCCCTGTGGTTCTCCTCACTCATATGACATAACCACACCTGGGGAACTTTAGAAATATAAGGATCTTGGGGCCCCATTCTGGATCAG
TCAGAATGTCGGGGATTGCGTCCCAGACCCAGGTTTGCTAATATGCAGCCAGGGCTGGGAACCACTGCCTCAGATGGAATACCACGGTTATGTT
GTTGGAGAGTTGCCTGTAGCTCTACAGCCAGCAGATGACTGACCTAGGACTTGCATCCAGGCTGTAGGCTCTCAAGCCAAATATCTCGACGGCA
CAATGGGCAACAATTCAGTTGTTATCTGGGCTCATATCTACCACCATGCCTAGTTTCTCTCTCCTAGTCTAAATCGCAAAGCCGCAAGTTCATT
TAGGGCTGTTTAAAGACTGTATGCTACACCAATTATGAGAATCAGAGCACAAACTTCTTGTTGCAGCCAAAGCAGTGATTTCCTACTTAAAAGT
TTCAGAGGTAACCTTTGCTTTTACCTGTCACCAACTTAAATGTAAGTTATCGTGCTTTTTCCTCATTGAGCAAAATCTTGTACTCACAGAGTCT
AGGACAGTGGTTCTAAAACTCTGATGCACATTAGAATCTCCTGGGGATCTTTAAGAGCTACAGATGGCCTGGCTTCCACCCCAGACATTTTTTT
TTTCTTTTTTTTTTTGAGACAAAGTCTCGCTGTGTAGCCCAGGTTGGAGTGCAGTGGCATGATCTCTGCTCGCCGCAACCTCCGCCTCCCAAGT
TCAAGCAATTCTCTGGCCTCAGCCTCCAGAGTAGCTGGGATTACAGGCGCTGGCCACTGCACCCGGCTAATTTTTTGTATTTTTAGTATAGACG
GGCTTTCACCATGTTGGCCAGGCTGGTCTTGAACTCCCGACCTCACGTCATCCGCCTGCCTCGGCCTCCCAAAGTGCTAGGATTACAGGCGTGA
GCCACCCCCCAGCCTCTGATTTTATTAGTATGGGATGTGACCTCGGCAATGGGAGTTTGTAAAGCTCTCCAGGTGATTCTGATGTGTAGCAGAG
TTTGAACAGTGCTTTAAGAACTTGGTCTGTGAGTTCACAGATGTGTTGACTTGAGAACACTGTGGAATCACCTCATGGATAGAACTCACTTTTT
AACTCTCTGACAGTGTCTCTTGCGTTTCAGCTGGCAGGATTAATTTGAGTTTAATTCCACCCATTTCACTACCTGACTTGGAATTGTACCTCAC
TGTCAAATGATGGTTTCTCCCCAAAGGTTGTTCTTATACTTACGCTCATGCAGTGATATTGGCGACATGTCTGTAAGATCCCTTTGTAAAGTTC
TCAGCCTCATTTGACTCTCTTATTTCCACTATAGAATATTTCTTTTTATGTTGTTTTACCTTTTAAAACTGTTTTTTCTTGAGACAGTGTCTTG
CTGTCACCCAGGCTGGAGTGCAGTGGTGCCATCTCGGCTCACTGCCCCCTCCACCTCCTGGGTTCAAGCAATTCTCCTGCCTCAGCCTCCAGAA
TAGCTTGGTGTGCCACCAAGCCTGGCTAATTTTTCTAATTTTAGTAGACACAAAGTTTCTTTTCACTGTGCTGCCCAGGCTGGTCTTGAACTCC
TGGTCTCAAGCAATCCGCCTGCCTTGACCTCCCAAAAAGTGCTGGGATTACAGGCGTGAGCCACCACGCCCAACCTAAAAACATTTTTATGATT
TTAATACATGTTGCTTTTTTTCTTCCACGAGCAAAATAAACACCTCTTCCACATAATGCATTATAGTAATAATCCACCTATGGTTCTTTTCCCA
AAATGGCTGACCATCAGCCTCACTATTCGGAGACAGCAGCTGATGTGCCTGTAGTGTTTAACGGCTGATAGCGTCTTACAGATAGATAGTGATA
TGGTGGAAAGTTGGAGATTATGTAAAGAAGAATTGACTCAGACTTTATCCTCAGAGATTTTAGTAAAGTCAGATTAATGTAATCAACTGGAAAA
TATACATGGATTATAAAAGTATATTAAAGCAGAAGTGTAAATTTGTTGGCAAGTTTGCAAATGTACTGGATTAATCTAGAAATCATTTTTGCAG
TAAGTCTTAAATAGATTTTTAAACAGAGATGTTATTTTGTGGATAAAAAGAGACAGGTAATTTACAAGTGGAACAATTCTTGATAAAGGCTGGG
GATGGCAAATGGAGTGTTGCAGGCCACTAGGCCAGCTAGCTAGGCTGGTATGAAAAGTATAAACTGGGTGTAGCAGTTAATGAGGAAGGATTGC
TGAGGTGGATAGAGCTAGCTATTGCATGAGATTTAATACCAAGAATACTTTGGAAGAGCTTTTTTTTTTTTAAAGGGATATATCAGGAGTTTCT
GTTTTCCTTTATGTGGTTGTTGAAGTCTAGTTGATTTCTAGTCTGTAATTTTGTTGATATTTCAGATCACCAAACAAGGCTGGTAAGATGGGTT
AGTTATGCTTCTCTTGAGGATAAACCTTTTCAAATTAACTCTGCTTCTTTAGGTTTAAGAATTTCTAATTCTGTCTGTCTACATCTTGCTGCTG
TAATCTCCAAGAAAAGAAAAAGGGGTTAGGATCCCTCTCTTTTGAAATGGAGGGGTCAGAGCAGGCAGGGGGCTGTCACTGTTGTTTCTCTTTG
TTCAGAAATTAGAACATGTGAGCAAGTGGTTCCACCCCTCATTTTGTGAGCTGTTTTCCTCTGATATTTCCTAATTTTTCCCATGACGTTTTAG
CCTGTTTCTTCACAAGATTGTAACCCCTGAAAGACCCCATTGTTTGACAGGAGTGCAGGCTTGTGCACATTTGCTGATCTGGCTGCCCGGCGTC
TGAGTGTGGGTGGTATACCGCCCGAGGGATCATGCAGACGGGAGAATCCGTGGAGCAGACTCCTGGTAGTTGCAAACACTTTATGTGTATTAAG
AGTTCATAGAATGTCTTTTTGTAGACAGAAGATGCTGCACTTTGAAAATGTTATTCACAAACTGAAATCTCATTGACAGAAGTAGGGATATTCC
ATTAGGCTAAGAAAAGTTTCTTAATGCCAAATCAAATGGATGAAGGCCTTATTCAAACTTCATTTCATCTTCAGATAGATTCAAACTTTTATTC
TTGCAGTTCACCCACATTCCTTGATCGCCTGTCTTGGATTTATGTGAGCCTTTGGAGCAGCAATCTCACATACTATGCATGGCAAATCCTAGTC
AATGCATACTTTTTCAGAGTGCAAGATGTTAATTGTAGTAAGAGAAGAGAAATTCAATAACAAATGTTTTTGAATAAAGGAAGCTGAAATTTAA
TACTAATGCTGTTTTCTCTAAATGAAGTTAATATATTATTGTTGTATTAACTTTTCAGGAGTTTGAGGCTGCAATGACCTGTGATTGCACCATT
CCCCTCCAGCCTGGATGACAGAGTGAGACCCTGCCTCTTAAAAAAAAAAAATTCTAAGGACTTCGGCTCTGTGTTTGATTGACAGTAATAGACC
AATAGAAGACATTGCATTTATATACACTTATAAAATAGCATTCACATTTCCAATCAGATTTACAGAATTCTCCCTTATTTTTAAATGTTCTCAG
TGGTAACAGTGACGTTGGGACTAGAGTCAGTATTGTGAGTCGTTTATGCACATCTCTTCACAACTCCATGTTCAGTGAGTCATACTGGCAGCTT
GAAATCAGCCATGGTGGGAGAGTTTACACCATGGAAATCAAACACTACAAATTGGTGCTCCTCACCCTCAGAGTCTGTTTTTAAACATTTAGCA
GCATACCACTGCCCAAAATGGAGTTGAGCAGTCAGGGAGTCTCCTCTTCAGGCAAGTCATGCAGTGCTTCCACTCAACCAAACAGCCAACATGA
ACATTTTGAAATGTAGGCTGTGTGCCTCCTGAAGAAAAGTCCTTCATAGCTCCACATTTTTCTGAGCCAAAGCCTTGCTGTGATCATAAAGCTA
TTCATGCTGTGGCCACCACCGTCCGTCCTCCTGGCTCTCACCTCCAGATGCCTGGTTTCACCCCTTTTTCTGTAGAACATCCTAGACCCACTCC
TATTTTAGGGCCTTTTCTGTACGGCAGTCGTTCTCAAACTTTACAGCACATCAGAATCACCTGCAGACTCTGCAAAAACAGACCACTGGGTGCC
TCTCTAGCATTTCTGAGCTAGTGACAAGTTTCAGGGTAATGCTGGTGGCCTTAGGACCCCGCTTGGAGAACCACTGCTTGAACTGATGGCCTCT
TGGTTTACCTCTCCCCTCCAGATCTCTGATCCTCTTTTTCTTTCTCTCAGTAAGGCACACCCTGACTATCCTATGTAAAATTGCAGCCTTTCTC
CTCCACCACAGTTCTAATTCCTGACTCCAATTTTTATTTATTTAAGTTCCAGGATACACGTGCAGGATGTGCAGGTTTGTTACATAGGTAAATG
TGTGGCATCGTGGTTTGCTGCACCTATCAACCTATCACCTAGCTATTAAGTCCCAAATGCATTAGCTATTTATCCTGATGCTCTCCCTCCCCAC
CCCCCTCTAACAGGCTCCAGTATGCCTTGTTCCCTTCCCTGTGTCCATCCGTTCTCATTGTTCAGCTCCCACGTATAAGTGTGAACATGTGGTG
TTTGCCTGACTCCCAATTTTTTTCAGTGCACCTGTCACCTGTTACCTTACAACAAGTTTCTTATTTGTGATTATCGTCTGTCTCTCCCCACTAA
CTCTAAATTTCACAAAGATAGGGAATTTTGTCTGTATTTTTTTAAAAAACAGGGTATCACTCCAATTATCCAGACTGGAGTGCAGTGGCATGAT
CACAGCTCACTCCACCCTCGAACTCCCGGGCCTGGGCTTGCGTCATTCTCCCCCATCAGCCTCCCGACTAGCTGCCACTACAGGCATGCACCAC
CATGCCCAGCTAATTTTTTGTATTTTAGTAGAGAGTTTTGCCATGTTCCCCAAGCTGGTCTTGAACTCCTGGGCTCAAGTGACCCACCCGCCTC
GGCCTCAGAAAGTGCTGGGATTACAGGTGTGAGCCAGCATGCCCAACCTTGTTTCTTGTTGTTGTTGTTGTTGTTGTTGTTGTTATGATGTATT
TCAAGCCTCTACAACAGTGCCTCGTGGCAGGTGCTCACTAAATTGTTGAATGAATTAATGCATGAACAACATTGAGATATGAATAATGTATTTC
AAATTTTGGTCCCAGCCTTTTAAATTCTAACTCACCAGAGAATCTCAAAACACTTTCTGCAGTTTTGTTTCTCACCTTCACTGCTCTTAGGATA
ATCAGGAATGGTTTCTGCCCTGGGCTGAAGTTTGGGAATGACTTGATGAGCCTTAAAATACCAGGTGCACCAAATTGTTGTTCTTCCATTTACA
TTTTAACCTCAAAGTCCCTAAGCCAGTGATCTTCAAACTCTGGTCTGCAGAAGTTCCCTGGAGATACTTCAGAGGCTCTGCAAACATCTAATTT
GCATTTCATTTTAATATATTTTAAACTATGTTTTATAACTATAATAAAAGAAAATGCACACAAATGTGCTACCTACCAAATTTTAACACATTAG
GACCACCAGCACATTGAATTGGACTCCCAAGTCAACCTTGAAATAGTGTTTTCCACTATTTTGGCTTACATATTTGAACTTCTTATAAATAAAT
GAATCATTGATTAGGAACTTCCATCCCAGTATTTTTCATATTCCAGCCTCATCATAAATTGAACAGGCAAATTCCTCTTGTCACACATATTCAG
CACACTTGATGACATGCCAGGCAGCGTTTCAGAGCCTCTGTGGCATCGAGGCTTCTGTGTGGATTAATGGCTAGTTGTACAATAAGTGAACACC
ACACACATCACTTAGTGATGTACATTCTGTTAGAGGATCATGTCTACCCACTCTACATTTTGGATACTCTTTTACCTTAAATAGTTGTCAAATT
ATCATTAGTGATTAAAATTTGAAATAATATCTTGACTGCTGCATTTTCTCCAAGCTTTTAGATTAGTTTTGAGGCATATTATGAGATTATCAAA
TGAAATGCTAAACAACTGTTATAATTTCCAATACAACTGTGCCTTACAATTCAAATCTGCTCATTTCCAGAATTCAGCTGCCAAAATTTCCCAT
TGTAAATTCCAGTGCAAAGGAATTTCAACTTCTTATGTTCATGTCAGCTTCGTTTCTCAGTTTCAGATAGTAAGTAGTAACAGTAGCAAATAGC
GAGAGATTTGTCTGAAAATTTGAGTTTTGAGAGCAGAGATTCAGATAGTGTTATCTGTATTTAGCAGTATCAGGATTCTCTTAATACAGTGAGT
GCAACCAAATACAGACACAGAAGTAAATCATATTCTGGATCACTTTTAAAAATCAATTGAGGCTGAGCCTCGTGGCTCATACCTGTAATCCCAG
CATTTTGGGAGCTCAGGGCAGCTGGATTGCTTGAGTCCATCTTTTCAAGACCATCCTGGGCAACATGCCAAAACCCTATCTCTACTTAAAAAAA
CAAAAAAAAAATTAGCTGGGTGTAGTGGTGTCCACCTGTAGTCCCAGCTACTTGCGACGCTGAGGTGAGAGAATCACCTGAGCCCAGGAGGTCC
AGGCTGCAGTGAGCTGAGATCACGCCACTGCACTCCAGCCTGGGCAACTGCAGTGAGGCCCTGTCTCAAAAAAGTCAGCTGATAGAAGCCTTAC
TATCTGAAAGCCCCACTCCATCTCACAAGTGCCATTGAATACATTATATGAAACAGTTCTAAGTTATACATTATGGAACATTTTGTAGGCCTCT
TTGCATATCTGTTTATTAATTTACTTAATGCTCTGTTGTAATTTGTTTTGTCTCCTACACTGTATTTGAGCTCTTTGAAAGTAAGAATTATGTC
GTAGCTTTGCTTCCCCAGTCCCCAGCGTTGCCTAACATGTGGTGGGTACTCAGCACATGTTTATTAAGTGTTGGAAGTAAATGCTCGGTGCCAC
CAAGTGAAAATAGCACCCAGGCAAAAGTTTTCTCAGCAAGCCAATTTGCTTCTATAGAAGGGTGCCTCTCATGGATGGAGCAATGGCCAGAGCA
CACTGGACAAGGAAGGGGAAGTGGGTCTTATCCTTAACCCAGCTAGTCCCTACTGCTGTGTCTTTCCCCTATTGGCTAGGGTTGCACCACACAG
TCTAAGCTAATTCCGATTGGCTGTTTTAAAGAGAGCAGGGGTGCGCGCCAGAGTGCGGCGTTGAGTAGTTTCGGCAGGAAGGACTGTTTGAGAG
CAGGTGACAGAGGATGACTAAGGACAGTGCAGGTGACTAACGATGACTAAGGACAGAGCAGTTGACTAAGGACAGAGCAGGTGACTAAGGATGA
CTAAGAGCAGATGATAGAGGCTAAGAGGGGGTTGTTTACTCAAACTAGGGGCAAGGAGACATAAAGAACGAGCAAGTTTTAAACTTTAAAATGG
AGAACAAAGAACAGGAGAGCTGAACATGCTGAACGTACCATATTGGTTCTTTGAAGAGGAATTTACTATATCCTACATAAGTGACACTGCATTC
TACTGCAGAAGGAAGCAAGCTCAAAAGGAACAATGAAACTTCATATTAAATACACAATCAGTCAGCTTTCATTCTAAGCACAAATTTCAGCATG
AAAAAAATGTAATGGTTCTGTGAGTCCTTCACTTGATCCCTCACATACAGTCTTCTAAAGTTTTCTGAATTCCTTTTACAAGTATCTTCTGACT
CTGACTCTTCCATGCCATCCTACTTGCTCTGAGCCTTGTTCTTCTCTGTCTCCCTTGCATGGACTGTGGAAGCCACCTCAGAAGCCTTCTCCCA
CACCCAATGTTTCTGCTTCTTCACATTGTTGCCATTGTCTCATGAAATAAAACCAAGCATATCCTTTGGTCTGCACTCCCTGCAAAATAACGGC
TAAACCTGTGCTTTCCAAATATAATACTCACTAGCCACAGGTGGCTGTTCAGCTCTTAAAATGTGGCTTGTCCAAATTGAGATGTGCCGTAAGT
GTAGAATCCACACCCTGTTTCAAAGACTCAGTATGAACAAAATTTGTAAAATAGCACATTAATACTTTTTATATTGATACCATCTTAAAATAAT
ACTTTTTTCTATAATATTAAGTAAATTTCTCTCTTTTTATTTGTTTAATGTGGTTATTAAAAAGTTTTAAATTACAGGTTTAGTTCACCTTGTG
TTTCTGTTGGCCAGCACTGGTCTAAGCTATATCATGACATTTAGAGTTTCTTATAACCTGGACTCAGTTACCTCTTTGGCCTCATCTCCCAGCC
CTTCCTGTCCCTTCCACAATTTCTCATAACCCCCAGACTCACCAACCCTGTTTATACTTACGGGTTTTAATTTTTTTCACATTCAGTTTCTTCT
TTCTGAAATGTCCCTCCTTTTTTAGGTGTCAAAAATCCTGCTCACACTTTTAGGACCTGACTTAATTGTCCCTTGCTGGTGATGTCACGCCCTA
TTTCCTCAAGCAGAATTAGCCACTCCTTCCCATATAGTGTCTCTCAGCACCTCCATTATTGCATTTGTCTTGCTGTTCTGCCATTAGGTGTCGT
CTTAAGTAGACTCCTCAAGGGAAAGGCTGCAACTTATTTCTCTGATGACCACAGGGCAGTACCTCGTGTCTACTAAGCACTTAATGGATGTTTA
TTACATTAACTTATGAACTAATTTTCATCCTGCTATACACACTGCTATTTTTATATACATCAGACCTTGATCATCTTAACCATTGGCCATGGAA
AAAATTAGATGCTGAGGTGTATACGACTGGACCTTTCAGCCTTAACCTCAATTTCAGGTTTTCTGTTCAATAAAATGGCCTCCTTTTTCTCACT
GGGTGGCTTATGATTAAAAAATTATAACTTTGAACTTAAAAAATCCATGAATATTGTACAGACTAAAAGTATAAAAGGACAGTATAAATTACTC
CTTTTTACCTATCTTGCTTATAGGGATTTCATATAAGATTTTGTTTGATTGAACATTTCACTTCAGAACAAGTTTGGAAAGCTACTGTTCTATT
CCTAAATTTCATTTACATTTCTAAACCAAAGGTTTCATTTTTATTTTGATGAGATGGTTTCTACTATCATCTTAGTTCACATAAGGCAACACTT
CTCAACTTTAGTTCCACAAAATTGGGAAGCTTTTCAAAAATACCTCCCCACTCAGGGATCCTTGTATAATTGAGCTGGAGGAAAGGGGCCCAGG
CACCAGTATTTATCAAAAGCACAAATACCTGTAATAAAATGATTCTGCAGTGCTTAGAAAACCAGGTAGTTAACTATGAAAATACTGAAGTTTT
AAATTGCTAATTTTTCTTTCTTTTTATTAACTATGCTGTTATTGTTTTTCTTGGCAGTCAAGCTAATCCAATTTAAAGTTTGTATTATGGTTTT
GAGACCTCAAACAGCATTTAATAGACGCCATCATCTCTGGACAATTTTCCTAAGGCTTCTGAAATTAAAAATTTCCTTTTCTGTCACTCTCTAA
AAACCTACATGCAGCAGAATTTTAATCTCCCTTATTGATAGAAATAGCTAACACAACACCTATTTCACATCATGGAAATGAGGACTGACAAGCG
CTGTGGGTTTAAGAAGTTTACATAGGTAGTGGCCTTCCTGGCACCCTTGTTAGCCTCAATAAACAGTCCTCCCCTGACAAATCCTCTGTAGCTC
TGCCACATTGATAAAATCTGACATTCTGGTTAGTCACTTTTTAATGATATTCTTTCTTTTGTTCTTGATATGTTAGAAACCAGCCCTTATTTTT
TTCAGTGTGTCCGAAAGAATTATCTTTAATTTCCTAATTCGGTAAAATGGGCTGCTAATGCAGACCTTATTCTTAGGGGGACAGGAAGAAAAGT
GACCTAATTTCTATATATCAAATACATCTTCCTTCATACATTTAAAAATGTATAGAGTTATAAAGGACCTTAAATCCTGTAGCGGAGAAACTCT
GGGGTAAATACTTTGGAAACATTGTGAAAAAATTCTATAAATTCTGATAGTAGGTTCCATTTCATTGCTCTGTACTATTAAAAAGTGCAGATGT
AAATAAGCATAAGTTTTGCAGGTTGTCAATTTGTTACAAGCGGCTGCATGTTCTTATTCTTCTGAAGTGTCATCTCTTTCAATGTGGGCTGTTT
AGGCCGAAAAAAAAAAACTCAGAAAAATACACACTTGGGATCAAACCCTTAGTCCAGAAAAAAGAATCAAAACCTGTAGTTAAGTTGGTTATTT
GCACTGCTGGTATGTCATCCTGTGTGAGACTGACGTCAGGCAGATTGAATCCACACCCAAACTTCGGGAGCGTGTGTGTGTGTGTGTGTGTCTC
TGTGTATGTGTTTTAAAGCCCCACTCCCAGAATATGAAGTGAGTATGAAGGGCACCACAGCAACCCCTGCATCTCTCAAGAATGTACCTGTTTT
TAACATGCTGGATCAAGTGTCAGCACTCAGTAGATGAGGAAGGGGTAGAGCCCGACTTGTACAGATTCCGCGGATGGTAAATCGCCCTCTGCCG
TGTTTACAATACAGCTGTGCATTCTTAATGGCTCTGCCGCTCCCATCTCAGGTAAAGTTTTCTTTGTACGTGATGAATGGCAGAGAATTCTTTA
GCAGAAGGAACCAAAGGAGCTGGGAGGAGGAAAGGCGAAGAGAAGACCGCAAAAGAGAGAGAAAAAGAAACACACAGCTCCACAGGGACATAGC
CTGTGCTTTGCAGTTGCTTGTGTGGATAGAAACCAGATGGTAAGCTCGAACACCCCCTTGGCTATCTGAGGAGCCAAACATATGGTTAATGGTC
GGGAGAAATAGCTTTCAGACCCAGGGCTTTGCTGACTGCCTCATATTACAGATTGTTACAGTCTGTCATTACATTGCCAACCTAAGTGCTTAAT
ACCCACTGCAGGCGCTGGGCATACACTTACAAGAAAGACTTTTAGCTCTTTGCTGGCTCTAAACAGAGAAGTCACATCATAGGGAGGCTGAAAA
TGTCTGGTGTTGGAGGTGGGGAGAAGGATAATTTTGTAGTCCAAACAACAATTTTTTTTCTTTTTTTTACAACCCTTCACTCCACTGAAGTCTA
CTGTTTTCTGAGAACCTTTTAATACTAGACTGGTTCTCTCACTCTCTCAATCTCTCTTGCGTGCACTCTCACTCTCTGTGTCACACACACACAC
ACACACACACACACACACACACACGGAAACGAAACCGAAATTCACAGCCCAGGCCATGTCCCCCAACTCCAGTGGATTTGACAGGCCTCGTTCA
CCTGGAAGCCACATCACTGACTCAGAGGCCCTGCATTTGAAGTTTCTGTTTGGGGAGCTTTGGGCAACAGCAGGCCTGTCTTATCTAAGGAACT
GCTGAGCCCATTCATCTGCTTTTCACGGCCCGTTTCCTTTTATTTTTAGCCAAATCCGTCAGGCCTGACAGGCGGGTAATTCGAGCCAGAGAAA
GGGCAGAGCTCCAGGTCCTTTCCACAAAGCAATTCTTCTCCTCCCTCCTGGGAGGGCTCCAGACTTGGCCTGCTTCACGCAGATGGATTTGTTG
AGTCATGTGAGATCATTTACACAAAAACACAGTGAGGCAGTGGAAATTAAAAAAAAAAAAACAAACAAAAGAAAAAGAGGAGGGTGGGCGAAGA
GGAAAGAGGCGAACCCAGGGGAATGTGCGCTGCCCTGCTGCTGAGGAGCATACGAATTTTGCAGTGAGGCAGCTAGACTGAGCAATGCCTAGAG
TCATTTGTGTATAAGGTAATGTCTAGTTTACAGAGTGGGGCGGGCCAGGCGACGACGGCGCCTCCAGCAGCTTGCCTCTGTTTAAAACAAGAAG
CTGTTGGGAAACTTTAAAAAAATTTTTAGAGCAACACCCACCAAACAAAGACCACACTGTTGACAGCGTTGGGTTCCAGTAGCGGTAAACTACC
AAGGGGCTGGTTTACAGAAAATGAGTTTGCTCTGTCATGTCATACAAGTTTGCTTTGTCCTGACGCCAACATCACACTCAGCCAGCAGAGGAGG
CAGAACAGGAAAATTGGAACCGTACTCTTAGTTAATGGTTTCCAGACATTATTTACCACCACTTGAACAGAGGGAGGAAAATATTGTCAACGAT
TGCTGCGAGGTGTACCATAATTGCGGTAGGTAAAACAAAGTCATTACTATGGTTTTCTTCCTCAAGAAGGATTGAGCAATGACTTCAAAATGCC
AAGTAAGGTATTATTTTTCCCTACCACACTATTTATTTTCCTTTAGCTTCTGGGGCATTAAAAAAAAACACCAACTCTCACATCATGCCAAACA
GTATTAATGTTAGTGAATCAATTTTGTTTACTGTTTATACCACAAGCCTTTAAAACATTCCATTAAAAAATTGTCTCCCGCTCATTCATGAAAT
TCTTTTTTTCCTTCAAATTTGCTTACATGCATTAGCTTTGTTTGGATAGGGACTTTTTCCCTTTTTGGCTGGAGTAAAAGAATGCATAAAAGAA
TAGTGAGTAGATTATCTAAATGTACATTCTTACAACAAATCTAATGAAAATATGCCGCGTGGATGAGACGGAATATTTCAAAACAGAAAGAAAA
ACAGAAATGGGGAGCATTTCAAAATAGTCACCCTGATTATGTTTGTTATGATGCATTTCTTCAATTCTGCATTTATTTAGAACCCTATTGTGCA
GCTATTTCTTTTAGCCACCCTAGATAGAATGTAGCTCAACATCAACATTGTTCCTCATATTTTGTTGGTATTTTAAAAGTTGAAACATTTTGCA
GTTTTTTCCAGAGCTAGATATTCTTAGGTTATTTAGACGGTTTTAGTGACCATAGAACTTCACGTATAAACTCCTTCTGTTAATAGCTACCACT
GAATTTTGATAGTAGACACTCAGATCACACCTTGACCTGGAGTAAACAAGGCACTGATGATATTGATGATAACATAGTCCAACACTGAAGCTGT
TCCACTTCTTCCAGGCAACTCTGATGGTCTTACTCTGTGTAAGTTACAGCTTTTACTTCGCTCAAGGAGAGAACTGGTAAATTTTTATGGTTGT
TAATTGTTTACATTTTGGGGACCCCAAGTTGCATGGTGCCACATTATTATAATGTGATTATGAGTTGGCATGATTTTAAGTCTACCCATATCCT
TGAATGTTCCCTCCCCGTCAGGATTTCCCAATCTCAGCACTATTGACATTTTGGACCAGTAATACTTTATTATGGGGTGTGTCCTGTACGTCGT
AGGATCTTCAGTAGCATCATTGGTCTCACCCACTAGATGCCAGTAGCACCCTCCCCCGCGTCATCACAACCAAAAATGTCTACAGCATTGCCAG
ATGTCCCCTGGGAGACAAAACCACTCTGCTTCAGAACTTCTGCTCCACATTTCTTACTCTGCTTTAGAAGCTCACGCTTTACGTCATGAGCTGG
CTGCTCAGTTACTTCTAAGATTGAATTAAAGGATATGAAATAATGTTTTAAGTTGGAATTCCAAGTTAAGTAAAGTTTTTGAAGCTGCAGGCAA
GAGTACTTTTGAGTATGGTTATGCAGAACAACCTCTCTCTTGGAATTGCGACCTTAGCAGGTGGATCCCCTCAGGACTCCCTTACAGTGAGTTT
CCCAGTGCTGTTTAACAGGAAATGACCCCACCGTCTGAGCCGAGGGAAGTCGACATTACCACAAACGGAACTTCCCCTCAGTCACCTCAGCAGA
CAGCCTAGGCCCCAGGCTCGCTGCCTGTATATCCAGCCCCAGAAAGAGGGCAAGGCAGCAGCCACAGTTCTTTCTTGGGAGCACAGCTGACCTC
CTCCCCATTCCAGCGGCTGTGGTCTCAGAAGCTGAAACTCTCGTTAATAAGGAATGTGCAGCTTTCCACAGCTAAGGCAAGCATAAATTTCCTC
TGTTTGTTTTGTGAGGACTAGTGTCAACTGCTGTAATCAGATCAGAGAGGACCCCAGGGTCAGCTGCTCCAACTGCTTTCTGTGGCTGTTCTCT
TGGCTGCTTGCTGTCGGAGAACGCACTGACTCCCTGCAGACCTCCCTGATGTGTTAAGGTGCTTGTGCCCTTGTTGCATTTCTTGCTGTCTTTG
CTTCTCTCATTCTACTAAAACATCACTGTGCAGTCATCCAGTTCTAAATTATCGAAAAGTTAATCCAAGGCAGATTCCTTTCCACAGAACTGAG
CGCAGACTTCGGTGTGCAAATTCCACCCTTGGGAAGGAAGGTGACCTGTTTCCTTCGGGTAACTCCCTGGACCTGCTGCTTCTGGCTAAGGGTA
GGTTTCTCAAGGCCTGGGCTACCCGTAAGCACTTTCTACCCCCAATGGCACTCTGTAATGAGGATGAGTTAGTGAAAGAGTCACAGCAATTAGC
TTACGCTACAAAGAACATAATTCTAGTTGTAGCCTAAATGACAGCTAGAATTGGCTACATATCGAACCTAAATATGGCAGAATTGCATTTAGGG
AACCCAATTCTCCCAGAATCGTATACTGAGCTCATGCAGCCTGGCCTACTTCTGACCACCCACCCCAAAAAGTCCTAAGCCTGGCCTACTTCTG
ACCACCCACCCCAAAAAGTCCTAAGGTATTATATTGGTTAAATATTTTCAAGTACATACGAGCTTTAAAGCACCACAGGGAAATTCTTAGGTGC
CAGAAGACATTGATGCCACTGGCCTTAGAGTGTTTCAGACCCAGCTGTAGGCTCTAGGCTCTAGGCGCTATGGTGTCTATACTCACACAGGAAT
TGCTTGTGAAAGCGGCCCAAGAAGATTTTCCCCAGTCTGTTTGGGGCTTTAAGTCTGTGTGTTGGGGCACAGACCAGGGGGATGGAAACCCAGG
GTTACACTGTTTGAATAGCTCTGGGGTTCATAGTTACACTACTACCTGCATGGTATAACTTCAACTGTTTTGGCAGAAACTGGTTTGAGAGCAT
TGAACGCCCTCACCCCATAGTGCAGCCAAACACTAGATCCCTGTGGAGAGGCTTAACAACCTCGGACATGGTACTCCCTCAAAAAGAATAACCT
GCACTAAGGAGATGCTGATAATAAGTACAGAATATGGCCACATACAGTGGCTCATGCCTGTAATCCCAGCAATTTGGGAGGCCAACACAGGAGG
ATCACTTGAGCCCAGGAGTTTGAAACCAGCCTATGCAACATAGCAAAACCCTGTCTCTACAAAAAATTATCTGGGCATGGGTGGTGCACACCTG
TAGTCCCAGCTACTTGGGAAGCTGAGGTGGGAGAATCACTTGAGCCTGGGAGGTTGAGGCTTCAGTGAGCCAACATCGCACCACTCCACTCCAC
CCTGGGCGACAGAGTGAGACCCTGTCTCAGAAAAAAAAAAAAAAAAAAAAAAAAAAGGAACAGAATCTAAGCCCCACAAAAGCAGAGGTTAACA
GTGAACCTTCAGCCCAACTAGAGTTTTCTTCTGCAGATTAATGAAAGTTTCTTCTCAACACGAACAAGCCATATAACCCATTCTGGCTTGCTAA
GCAGTTTCAGTTGTAAGCACTAGCCCCAATTAAGGTAACATCCTACTTATAGCTTGTCCCCAAGATCTTCTTGAGAATATTTTACTTCCTTAAT
TAGAAACTTTTTATTGGAGAGATAGCTGGCCCCCTTAACGCTTCTTTAACAGTGCTCCCATTGCAGTGGATCACTCTGAAGTAACCCATCAGGC
TGTCTGTTATTTCAGTTTCTCACCAAATCATACGAAATATCTTTGCCTGAATTGTACAGCAACCCCCTCCCCCCGCCCCGCCAAGCGAGAGGGA
AGTTGAAATTATTTGGAGGGGTCTGGGATTCTTTCAGCATAAAATCAAAGAAACCCAGGTAAATAAGACATGGTTAGTAAACATTTGTTCTAGA
TGAATATAATTCAGCCTTCTCAATCCAAAGTGAACAAATGGATCAAGAGTTGCCTGGAATAGCCACTGTGTACTCCCTTTGCAGTCACGTGCAC
TATTGGCAAACATCACTTGAGAATGCTAAACTTAATGCATCATTCCTTAATTTATTAATAGCACAATGAAATCTCTTTTGTTTAGTTTTTTTTC
TTCTAAAGTGCGCAAAAAGCTGTCACCAGTAGTACCCAAATAGTGCCTTGCTGACCTAAAAGTGTGCCATGCTTCTTAAGAAAAAACTTCAGTA
TTTATTCTTAATTTGCACTTCTATAAAGTTTAATTTCAAAATATTCTGACCAAGCCAGAAGTAGATATGAATATTGACATTAAACCATCAGAAG
AGGAGAGAATGAAAGCAAATAGGTTCAACTGGTGCCAGACTTACAGGAGATCTTCCCAAACATTATAATTAAATTTGGCATATATTGAGTGCCT
ACTGTATGTATGTAAGGCAGCGGGATAGGTGCTGGGTATTTGGGTACTACTACAAAATAGACAAGATCTCTTCTTGTGAAATGCTCAAAACAAT
CCTCAAAAGAATCTCCAGAGATATAAAGAATTTCCCCCAGCTTTAGGGATAAAGGAACAGAGACTGAGAGAGAGCCAGTAACTTGCCAAAAATC
ACATTCCGAGAGGTGGAGTTGATATTCAGACTGACTTGGCCCCTATCTCTCTTTGCCTACTCTTCTCCCTGGCTTTCTGTGGGCAGTGAAGCCC
TAGCAGTACAACCCAGAGGCCCCAAGAGGCAGGTACAGAGTTTTCTCTCCCGGAGCTTTACACGAACAAAGAATCCCCTGACTGACATGGAAGG
TGTGCAGTAGAAGAGGCAAACTAGCCAACCAGACCCAACTCCTACAAAATTTCCAAAGTATTAGCATGAGTTTCCAACATTAAAACAGCAAATT
ATTTTACACAGTCTCTCAACTCCGTTTCTTGAGTATCAGAAGCTCTTGGCACATGCGGCTTGTTCCTGCTTTGCTGGGGCTACAGCTGAGTAGC
AGACTCCCCCGATCCCCCATTCCTCACTGTCTTCCCACACCTGACCTTTCACTCATCTCATTCTTTCTTGACCCTTGCAGTCATCTGAATTTGT
GACCCCTGGTTTAAATAGTCACTTAGGCAAGCGAAGTGGGCCAGGCCAAGTAACCTCTTGGGGGCTCCTCTGTGTTGTGCCTTTAAATTGAGAT
AGTCCTAAATCACTGGAGACTGCGATTTACATTTTACAGTGATAGCCCTGAATCTAAAGATTAGATATCCTGACTTATCATTTGACCAAGTGCA
AGAACCCCCTAGCCTGTGTGCCAGAAGTGGGTGTATGAAAGTGATGGACCCTCTACCACTTGTCACTCTTGGCCCTTCCTGGCCTCTGTTTCTC
CCCATCTGGCTCTCAGACTATTTGTGTGCGTGTGTTACCAACAATGTGTCTCTCTTTTATAATTTATAATCCTCTTGTTTCCTCCTCTTTATTA
CCATTTCCTATTGAACCATAACTCATATATTAGTTAAAATTTCAGAACATTCATTTTCAAATTACCTAACCTATCCTTGGGGAGGAAGGCTCTT
GTTGATGCTTGATGGGAACATACTCTTGTTGTTCTGGCTTAGAATGTCTGTTTTCCATGGTATGCTGTGAACGCAGCGCACAGGGTAGCAAAGC
AGAATGGCAAACGTCTGGTCTCTTGGGGCAGAACCATGTTATAGTGTGTTTGCTGCCTCCATGTGGGGTGGCGAGGACACTGAAGTCATGGAGA
ACCACAGCCACACATCTGCTGTAACTTTGAGGATGGACAGCGACCTGCAGGAATCTCCTTGGCAACCTCCACTCTGTTCCTCTTGCCTCTTCTT
TAGGAAGCAGGGAATGAGCCAGCCACTTGGAAAGAAGCTGTGTATGCATTGGCGGAAGCGGGCAGGAGTCTAGTTTCTTTGCCTTTGGATCCTT
CAATCCCAGCACCATGCCCCCTATGGTAGAAGGGAGGGATTAGCTTTCTGTAATAAAATGACCTTATATTTTTTCAGTTGAAAGTCTCCAGTTG
AAGATTGTCCAGACTTAACTTCAAAGCCATGTGAATTACTCCACCCCTTAAAATAGAGTAGAGGAAATTTGGGATATTGCTCTCACCTGTCCAT
CTGATTTCCCAAGTTTCTAAGTCCTAGATGCAAGAGCATTTTTTTCTAGACTTCAGAGCCGGTTCACCTTGTATTACTTCACTTACTCCTTCTC
CAAAAGCTCTTGGGCCTGGCATAGGCCCTCATGCCCCAGCCCTCTGCCTAAGCTGCTGGGCGTGGTGGTGGGAAGGGTAGGAACAAGAGTCCAT
CCCACAGACTGGCCTCATGGTACGTTATGTCTAAAAGCTAGTGAGACCTTTAACACTGCGAAGAGTGACCTGCTTGCTCTTGTTGGCCTATCTA
TCTCCCACAGGAGTGCCTGAGCTACCCAGCTTTGACATATATTACTGAAGGCTTCTAGAATGGACATCATAGAAGGGGGTGGGAGTTGGAGTGG
CGAGAAAAGCCCAACTCACCATATTCCATTCACTATTTGGCAGTATTAGTTGCATTTTCAACGGTCAGGAGAGCACACACACACACACACACAC
ACACACACACACACACACACACAGCCCTTGACACTTTTAACCTTCTCTTTAAAGAGCATAGGATTAAAGGTGGCTTATGTTATTCTCAGCATAC
CTCTATACCACATTAACAGTGATCCAGGTAAAATAGGCTTCTCTCCATGGTACATGGAGGTTGATGGTAAATAAATAGTGGTGAATCAGGTACG
CCTGGTGTTTTCTCCAACTCCCATTTGCTGACACCCAAGACATCAGAGTATAACCCTCAGAGAAACCATAGGATGTGGAATTGTCACCAGGAGC
CCAAATCTCTGGGTCTCTTCCATAGCGAGGACCTGGCTGGTGAGGGGCACTTGGCCATGTTACCAAACCTATCTCTGAGTGTCTTTGTTTGCTT
GCCGCCCTTTGTTTTTCTCCATTCTTTATATCTTTCATCTTTTCTTGTCATTTCTCTCTTCTTCTTAGACTTACTTTGTCTCTGCCTTTTTTTT
TTTTTTTTTTTTTTTGAGATGGAGTCTCCCTCTGTCACCCAGGCTACAGTGCCTCTGCCTCCTGGTTCAGGCTATTCTCCTGCCTCAGCCTCCT
GAGTAGCTGGGATTACAGGCCTGCCCCACCATGCCCAGCTAATTTTTGTATTTTTAGTAGAGATGAGGTTTTTCCATGTTGGCCAGGCTGCTAC
TCCTGACTTCAAGTGGTCCACCCGTCTCAGCCTCACAAAGTGCTGGGATTCTGTAATTCTGCACCACCTTTCTTCTTACCCATGCTTCTATCCA
CCTGGAAAGACAAATTATGTTTCATTAACCTTTTAGAAGTCTAAGATTGACCATTGAACCAACTGTTAGGATCAGCTGAGTCGAAGTCACACGG
CCTTATGGGATTAACTTTCTCAAGGCACCCTTTAAAGACCTCTGTAGTTTTCCTTTTGTTCAGTACTACAGGAATCCAACTTCTTAAAACAATA
AAAAGAAACATTGAATAATAAACTAAAAAAGAAAACATTCTTGATTCCACCATGGTAACTCAGGTATCTTTTTCCCTAATGCTTTATATTTAAG
ATGCAGTCTCCTTTTTTAAGCTGTAGATTTTGCTAAGCTTCTTCATAAACTGAATTGGCTGCAAAACAACCAGGTTCCCTCGATTTCAAAGGAG
ATGGAAAGTACTTGATAGAACAAGGCCAGTCTTTTTGGTTGCTGTCCAGAAGTGAACTCACCCAGTTTCTAAAATTGTCATCTGTTCTCTGAGG
CGTTTTCTGTTAAATGGTGGTCTTGGTGTGTGCATGTGCGTGTGTGTGTGTGTGTGTGTGTGTGTGTGTGTGTGTGTTGTTTTGGGGTGTGTGT
TGTGTTTATTTTTTATTTCTGGTCTTTCACATGCTTTATTTCCATAGCGTCTTGAAATGAAGTCACTTCTAGTCCAGGGTCTATTGCTTATTAT
AGCAAGAACCAGGAAAAATTCTTTTACCTCTCTGTGTCTGGTTTCCAAATTAGGAAAA
HUMAN SEQUENCE—mRNA
GCGGCCGCCGCCCTCGCCACCCCGCCTGCCCACTCCCACCGCCCCCCCGCTCTCGCTTTCCCCCCGGCCTCCCCTCGCCCCTTCCCCTCCCCCT
TCCCGGCGCACTCGGGGGGCTGGGAACGAGCTGCCATGTGATGCGCGTCCCCTCCGCGAGCTTTCGGTGACCCACGAACTGCCCACCTCGCCGG
CTGCCGCGACGGGGCTGCGAGCCGGGAAGACGCGGGGAAGAGGAGGCGGAAAAGGACGCAAAGTTCTCCGGCGAGCGCATTCATTCACATAGCT
CCCAGTTTTAACATTTCGCCACCTACTGAAGACATCATTTGGGACCAAGCTGATGAGCCCTTGAAGCACAGCAGATAACAGTGTTGCTGTTCAT
CATCTTTGCCTGGGAAGTTGAATGATAAAGCCAGAAGAAAGCATGCTTTCTGATCATTTGCAGCTGTCTGCTTCAGAAAGTGAGGGCTCCAGGA
ATGAGGAGAATCTTCAAGAACTTCCTCGCACTGTGACATGTCTGATCCCTTGCTCCCATCCCTGCAGCATGAACAAGGTGGACACTCACTCACC
TGTCACAAGGTTGCCCCACAAAACTTTGGGGTCCATGTCTGAATGGATTGCCAGAGCCTTCTCATCTCTCCCTTCGCCCAGTTCCCTGCATCCT
AAGACTCGAAGGCAGCACAGGACCTGGAAAAATTACATGGTGTGAACGGCATGTCTGTGGATGAGAAGCCTGACTCCCCCATGTATGTGTATGA
GTCCACAGTCCACTGCACCAACATCCTCCTGGGCCTCAATGACCAGCGGAAAAAGGATATTCTCTGTGACGTCACTTTGATCGTGGAGAGGAAG
GAGTTCCGGGCCCACCGGGCTGTGCTGGCCGCATGCAGTGAATATTTTTGGCAGGCGCTGGTTGGACAGACAAAAAATGATTTGGTGGTCAGCT
TGCCTGAGGAGGTCACAGCCAGGGGCTTTCGGCCGCTGTTACAGTTTGCCTACACTGCCAAGCTGTTACTCAGCASAGAAAACATCCGCGAGGT
CATCCGCTGTGCTGAGTTCCTGCGCATGCACAACCTGGAGGACTCCTGCTTCAGCTTCCTGCAGACCCAGCTCCTGAACAGTGAGGATGGCCTG
TTTGTGTGCCGGAAGGATGCTGCGTGCCAGCGCCCACACGAGGACTGCGAGAACTCTGCAGGAGAGGAGGAGGATGAAGAGGAGGAGACGATCG
ATTCAGAGACCGCCAAGATGGCTTGCCCCAGGGACCAGATGCTTCCAGAGCCCATCAGCTTTGAGCCCGCCGCCATCCCCGTAGCACAGAAGGA
AGAAGCCCTGCTGCCCCAGCCTGACGTGCCCACAGACACCAAGGAGAGCTCAGAAAAGGACGCGTTAACGCAGTACCCCAGATACAAGAAATAC
CAGCTTGCATCTACCAACAATGTCTATAATGCATCATCACACAGTACCTCAGGTTTTGCAAGCACATTCCGGGAAGATAACTCTAGCAACAGCC
TCAAGCCGGGGCTTGCCAGGGGGCAGATTAAAAGTGAGCCGCCCAGTGAAGAGAATGAGGAAGAGAGCATCACGCTCTCCCTGTCTGGAGATGA
GCCTGACGCCAAGCACAGAGCGGGGGATGTCGAGATGGACCGGAAACAGCCCAGCCCTGCCCCTACCCCCACGGCCCCAGCTGGGGCCGCCTGC
CTGAAGACATCCAGGACCGTGGCCTCGCCCTCCTGCTTAAGGTCTCTCTTCAGCATAACGAAAAGTGTGGAGCTGTCTGGCCTGCCCAGTACAT
CTCAGCAGCACTTTGCCAGGAGTCCAGCCTGCCCTTTTGACAAGGGGATCACTCAGGGTGACCTTAAAACTGACTACACCCCTTTCACAGGGAA
TTATGGACAGCCCCACGTGGGCCACAAGGACGTGTCCAACTTCACCATGGGGTCGCCCCTCAGGGGGCCTGGGTTGGAGGCTCTCTGTAAACAG
GAGGGAGAGCTGGACCGGAGGAGCGTGATCTTCTCCTCCAGCGCTTGTGACCAAGTGAGCACCTCGGTGCATTCTTATTCTGGGGTGAGCAGTT
TGGACAAAGACCTCTCTGAGCCGGTGCCAAAGGGTCTGTGGGTGGGAGCCGGCCAGTCCCTCCCCAGCTCGCAGGCCTACTCCCACGGTGGGCT
GATGGCCGACCACTTGCCAGCAAGGATGCGGCCCAACACCAGCTGCCCGGTGCCAATCAAAGTCTGCCCTCGCTCACCCCCCTTGGAGACCAGC
ACCAGGACTTCCAGCTCCTGCTCTTCCTATTCCTACGCGGAGGACGGGAGCGGGGGCTCACCCTGCAGCCTCCCTCTCTGTGAGTTCTCCTCCT
CGCCCTGTTCCCAGGGAGCCAGATTCCTTGCCACAGAACATCAGGAACCAGGCCTGATGGGAGATGGAATGTACAACCAAGTGCGGCCCCAAAT
TAAATGTGAGCAGTCTTATGGAACCAACTCCAGTGACGAATCCCGATCCTTCTCGGAAGCAGACAGTGAGTCGTGTCCTGTGCAGGACAGGGGC
CAGGAGGTAAAACTTCCTTTTCCTGTAGATCAAATCACAGATCTTCCAAGGAACGATTTCCAGATGATGATTAAAATGCACAAGCTAACCTCAG
AACAGTTAGAGTTTATTCATGATGTCCGACGGCGCAGCAAGAACCGCATCGCGGCCCAGCGCTCCCGCAAAAGGAAACTGGACTGTATTCAGAA
TTTAGAATGTGAAATCCGCAAATTGGTGTGTGAGAAAGAGAAACTGTTGTCAGAGAGGAATCAACTGAAAGCATGCATGGGGGAACTGTTGGAC
AACTTCTCCTGCCTTTCCCAGGAAGTTTGCCGAGACATCCACAGCCCCGAGCAGATCCAGGCCCTGCATCGGTATTGCCCTGTCCTCAGACCCA
TGGACTTGCCCACGGCCTCCAGTATTAACCCTGCGCCCTTGGCTCCTGAGCAGAACATTGCGGCCTCCCAATGCGCACTGGGGGAAAACGTGCC
CTGCTGCTTGGAGCCAGGCGCGGCTCCCCCCGGACCCCCCTGGGCACCCAGCAACACCTCCGAGAATTGTACCTCTGGGAGGAGACTTGAAGGC
ACTGACCCGGCAACCTTCTCAGACAGAGGACCTCCTCTTGAACCCAGGAGCCAAACAGTGACCCTGGACTTCTGCCAGGAAATGACTGATAAGT
GTACAACTGACGAACAGCCCAGGAAAGATTATACCTAGTCACTCGGCTCTGCCTCCCAGTCCGCACACCTCTCCCATCCAGGCGTTCTTCAGTC
AGCCTGTGGCACTGTTCATCTGCTGTCCCGAAGAAACCGAGAACACATTTGGTGCACACTACAGCGGTCTTAGCAGCAATACTGTTCCGAAGTA
TCCTCTCCTCTTCTCGAGCAGGAGTGATAGTTACCTTCACAATGGTGCTACCCCTTGCCCAGGCAAGGAAAGACAGCAGTGATGACACTGTCTG
TCTGTGGCTCAATTTCAGTCTTCACAGGGATAGACTACAACACCTCTAGGCCCCAACCACGGATTTTTTTTCTCAGTGGCCCATGTCACAAACC
CTATCTCAGGAATTTCTTCTGAATGTTCAATTTTTTTCATTCAACACAGCTTCTATACACATCAAAGTTTTATAGCTAGACTGTACATATTATA
TATAATATATATATAAAAAATATATATATATATATATATCCATATGCAAAAGTCCTGCATGCCTCAACTTTCTCATCCTAAAACTGGAAACTTA
TTTCTCATTTAGAAACAGGTTCCAACATTCCTCTTCTTTTGTCTCTGATGCTAGAACTAGTTTGGTAACTGTTAACGTGGTCATTTTTCTTGCT
TCACAGTTCAATTTTCAATTCGTACTTATTTATGGACAAAATTCAGTGTTGGAAGCTTTTTCCCAAGGTTTTATTTCACATTTCTTTTTCGTTT
GGTTTGGTTTTGGCACCTCCAAGTGGTGTCATTTGAGCATTCTAGGTTTGTTTTTTGTTTGTTTGGGGGGTTTTGTTTGTTTTTGTTTTTGTTT
TTGTTTTCCTTGCAGATACTGTACAGTAATGGTCAACTTTGCCACTTGCACTGAGTTTTGGGTCAAACCTATTTTCTTAAATGAAGTTGTAACT
TCGGTATAACTCAAGTATACTGTATATTCTTTGCTTTTAGTTAAAAAAGTAAAACATTTTAGCTAATTAAAAAGCACTCAGGTGATAATTATGT
AGGAAAAACAATCTTGCCAAATAATGAATTCATCCTAGGATGTGTAGACAATAATCTGCTTGAATATTTTTATATTTCACCTCCTCCCCACCTT
TCCCTAACCAAAGTTTAAACGCAGATAGACAGTTCAGAGTTGATGCTCGATGTTCAGATTCCTAACTGGGGAGAGAGTTTGGACATCTCACTCA
AAAGTACATCAGAAAAACAGGAATCCGTGATTTTATACCAGAACTCAGCAGGCATTGGCTCCTAGAAATCAAGTTAGAAAGTTTTCACCCAGGG
AGTAAGTCCCATTCATTTCAACACGTCCTGAGGCCTCGGCTTGCTCTTGGAAGTGGTGTGCAGTAGGACCTGCTCCCCTGAAGGACGGGGCCAA
CCAGCCACTCGCTTTCCTGCCCACGCTTGCCCTCCCAGGACATCTGGCCTGAGGGGATTTGAATCACAGCCCCGAAGGTCCTGCCTTCACCCCA
TTGGGAGAGAGCAGGGCATCCTGGCATCTGCGATCCATCCCTGACACAGGCTGACACATTCTTTCTCCTTTCCTTCTCCAAACGCTTGGAGTTT
TCTTCTGAGGTTTTTCTGCCAGTGTCTTGTCTGAAGGCAGACTTCATTCTGAGGCTTTGGACAAGCTATTCACCGGGAACCCTCCCTGTCCCCT
TCCCCAATCACACACATACCCTACCCTCACCTGATGATAATTTTCTCTTCTTGCTGCAAAACTGGTTGGCTTGCAACCCAGAGAGAGCACCTTC
CCTTGGCTCTGGGGGCCGTGTTGGCCCCAGCCACGTTTACACGAAGGTGTGCCCCACAGGAGGAGGAATCAGCTCCCTCGCTCCAGTGGCCTTG
GGTCCGGGTCTCACTGAGCAGCCCGAGGGCCACTCCAGCCCGGCTGGCCAAGAGAGTCCTGAACGGTTTGATGTGGGCATGGGGTGGTGGGCAG
TGGGGAATAGATGCTTGACTTTGTTTCTTTATTTGTGCCATTGTTTGGACAATATTAAAGCTGCATCTAAAAGGGGAAATTAGTATATGATGTA
GGCTAAAAGTGAAATCATAGTAACATATGTTTTAGTATTATTAACTTTTTTCTGTACAAATATTAGCACTAAATGTTTAAATATGTATGAATGC
CAGAAATTTGTCAGTTCATGCAGTAGGATAAAAAAAAAAAAAAAAAAAAAAGGGCTTTTCTTTTTAAACAGTTCCACTTTTAAAACCTGCCTCT
GGGTTTTTGTTTTTTCTTGTTTGTGTGTGTGTGTGTGTGTGTGTGTGTGTGTGTGTGTGTGTGTGTGTGTCTGAAACAGATCTTGATAAAGCTC
TGTGTTGGAGCTGCTGGTTTTTGTTATGGTTGTTGGAATTTCTTGGCCTACTAGGACAGTTCTGTGCTTCACCATGAGGTTTGCCTTTGTGGAA
AACTCGTGGGTGACAGTGAGAATATAAACTCAATGTCAATCACGTGATACTTCCGCAGGCGTGTGTTACAGTGGAGTCAGCTGACAGTATTTTG
CTTTTTAACTCTATTGTTGCCTTTCCAAGTGACCTCTCCTCTTCTTTTAAAAAAAGAACACTTTCTGCTCATATCATAACCAGGTCCAACCCAG
CTTCTTGTCATCAGGTTTACCCTGGTAACAACTCATGTGCAACTGCTAGTCTTGACCACATTCCATCCATTTCCTCAGGTTTCTGTGGTTCAGT
AGCCCAGACCTGTTTGGCAGCCATTTCTAGCAGGGGCGGGGCCTCTTTATTTCTCTCCACCCTATCTCAGACCTCACCTTCCTCCCACCCACCC
CTGCCTTGCTTTTCTTCCTCTTCCCCCAACCTAACTTCTGCCATGGGAACTGGTTAAAAACACTGCTCTAAAAACCATCTTCCAATTTCATAGA
GATTTCTCACAAGTTATTTCATTCATAATCCACCATCAACAGTGACTACCTTCGTGCAGTTGTTCATGTGATGTGTGTGTGTCTTTTCCTATTC
AGAACTATGTGCTTCTCAAAATTATTTCTGGGTTGATTCAAAGGGAGGACTTGCTGGGGACCAGAATCCAAACGGCCTCAAGTGGAATTTTAAA
ACCTACCCTGTCTCTTTTCCCTGGCATCCCTCTGTCAACCCCACGCCTTTTAGGAAAAAGAAAAGTGAGTGAACAGCAAGGAAGAGTGTTTCCA
CAGTACAGTAACATTTGGTTGTTCTTAAGGCTCTTTTCTTACAAAAATAAGAGACCCTCCAACCACGGGCTGTTTAGGAGGATGCCTGCTTGGG
TCTCCAAATGGCTGGGGTAGGAATGGTTGTTGGGGCAGACCCAGTGGAGGTGAGTGACCCTGAGACTAATGAACATCCCACCTAAATCCAGTCC
TCCCCTTGGATCTGCCTTTGTCCTGCTTCTGTATCCAGGCAACCTCTTTTCAAGTTGGTCAGGCTTTCGACACGTGAGTGATTTCCTGTATGTG
TTTGTTTCTCTGCGTTACCTGGGGGTGCCTTGATTAAAATCGAACTTTATTACATACTGATTCTGGAACAAAACAGTTAGAAAAACTTTAAACT
TTAAAAAAAAAAAAACCGACAAAGTTACGAGGCCATCCTGCTATTTATCTTCTGAGTTCCCAGCAATGACTCAGGCATCAGAGATGATGCTGCA
GTGGAAAACCTGACTCTGTGTCTCTGCAACTGAATGTTGTGCGAGTAATTTATTAACTGTCTTTCTAAAGCTTTCCTGCTTTTAACATGCACTA
TAATTCGGGATGTAATCCTTACATTGCTTTTCCAAGGAAGGGAACAAAAGTCTAGTGATTAGTATGCCAACTGCCACTACTCCTTCAAAAGGAG
CCAGGACCAGCGACAAGACTCATGAGAGGACTGGCTAAAGTGAAGTGTGCACAGTGTGAAGTTTAATGCTGTTGTCAAGAGGCCTAAACCCACA
TTTTCTCTTTTAATATTTTATGATTGCCATCAAAGAAGAAGAAAAAGAAGGAACAGACAAAGGTTTGAAAATGATAACCCTGTTAAGACACCAA
AAACTCCTGTCCCGTGAAGCTGCTTGACATCCTGTGGAGTAGCATAATCCTCTCAAAATGAGGAAGAGCTGCCTGCAAAGCTTTCTCAAGTCCC
TATTTGGCTACCTACTTCTCTACATTATGCCCCATTTAAACTAGGAGCTGTCTTAGAAATGACTTCAAACTGCTTCACTATTGCTTACAGTTTA
CGAGGAGTCTCAGATCCAGAACGAGCAAGAATCAAGTTTGGTCCTCAAATGACTGTAAATAGACTAAAGAACAAGGTGTTTTTTGTTTTTGTTT
TTGTTTTCTAACAATAAAGCTGTTCGTTCTATCATGAGTTAGTGTTTCTTCCCCAAACTGAACACTGTGTTGGAAGTGCAATTTCTGGTGAGTC
AGTCCACAATACAATGCCCTGTGTGGAGTTGGTATTCATACAGGAAATCTGTGTGCACGAGGCATTGTGTGTTGAAAGTGTATGTTTATAGTAC
TGCCTGAGCCATCTCATGACCCCAGCGTCCAAAACCGATGCTGTAGAACAGAACATATCTGTCACAAATAGGTGTGTGCAAATAGCATTTGTAC
ATACAAAAGTCTCATTGTGGCAGATTGAGCATAAATTATTCAACTGACGGTGCAAAAACATTACTTGCAAAGAAAAGTTTATAGTATTTTCCTA
CACTCCACCCTGGGAGATGATATTTCTATCAAATGAATATCAGTGCATTTTAAATGTAATATGAAAACGATGCTGCCATTTTGTGAAGAATACC
CACTTGGTTGCAGACGCCAACTTTCATAGCTTTGATTTAATGTTGTGACACGGTGTATGCATTTTGCTGTCAAGCAATGGATAAACAGCTCTGA
CTTTCATTCTCATTCCAGTTTATTGACCTCAGATAAAACACTGGCCCTTCTTAGAAGCAGAAGTGTGCACCAAGACCATTCATTTCAGGTAGAC
TCACATTCAGTGCCAAGTGCTCCCATGGGAATAATCAGACGCATATGTTGCGAAAGAGTGAAGGGACTTGGACAAAGAGGGGTTTTCCTACAGA
TGGATGCTCAGTCTTCTACCAAAACATGTTTGGAGGCAGAACTATGACCTCCCCTTAAGTCCTAACAATGTATTTTGTGTGTGCAAATCCTGGG
ATGCCCGTTTCACGCTCTGACATAAAGACATGGCACCTCTAGTGAGTGATCAGGAAGATTCCATATGCATTTGGGAGCTTCAGGTGCTTGTTAG
ACACAGTGAGCCATTCAAGGCAAGCACCACCTTTGCTAGTGAGGCCAAGAGAGCCTGTGACAATTTGACAATTTGTTCCAGAACCAGTCTGATG
CAAGTGCACCTCTAATATATCCCTTACAAACTCCAGAGGCCATATTCAAAACAGGGTCTTCTCAGTGTATGCAAGGGGCTGCAGCCCCTCTTCT
CTTCCTCCCCAGGTTGAACAATACGGACAGTTTTCACACATATCTACCTGTATAACCCTCTGTACCTCTCATAACTGCTCAACGACTGTAACAG
GTTACATCAGGTGTTTTTCTACATACTTTTTACACAGATTCTATGCGATTAATGTAATTTAATTCAATGCATCATTTTATTGTACTAGTTCTTA
GGCTTGTCCTTATTTTTTTCTAAGTGATTGTGGTTTTTCTCGTGGTTTTTATTGTAAAAAATGAAAGGCTGTTGATGCTTATTCTCTGTAACTA
AGAATTTTACCTTTTGGGGGAAAAAAGCATTGCTATGAACTAATGGAATTGGAACTTCATTTACTCATTGTAAATACACTATTGTGCAAAAAAA
GTTTTCACTCAATTGAATTGCTAGTGTTAACTGAATTTTGTCTAGACACCATTTCTGTTGATGAAATAAAGACATATCATTATGCATTGTAAAC
TG
HUMAN SEQUENCE—CODING
ATGAGAAGCCTGACTCCCCCATGTATGTGTATGAGTCCACAGTCCACTGCACCAACATCCTCCTGGGCCTCAATGACCAGCGGAAAAAGGATAT
TCTCTGTGACGTGACTTTGATCGTGGAGAGGAAGGAGTTCCGGGCCCACCGGGCTGTGCTGGCCGCATGCAGTGAATATTTTTGGCAGGCGCTG
G
TTGGACAGACAAAAAATGATTTGGTGCTCAGCTTGCCTGAGGAGGTCACAGCCAGGGGCTTTGGGCCGCTGTTACACTTTGCCTACACTGCCA
AGCTGTTACTCAGCAGAGAAAACATCCGCGAGGTCATCCGCTGTGCTGAGTTCCTGCGCATGCACAACCTGGAGGACTCCTGCTTCAGCTTCCT
GCAGACCCAGCTCCTGAACAGTGAGGATGGCCTGTTTGTGTGCCGCAACGATGCTGCGTGCCAGCGCCCACACGAGGACTGCGAGAACTCTGCA
GGAGAGGAGGAGGATGAAGAGGAGGAGACGATGGATTCAGAGACGGCCAAGATGGCTTGCCCCAGGGACCAGATGCTTCCAGAGCCCATCAGCT
TTGAGCCCGCCGCCATCCCCGTAGCAGAGAAGGAACAAGCCCTGCTGCCCGAGCCTGACGTGCCCACAGACACCAAGGAGAGCTCAGAAAAGGA
CGCGTTAACGCAGTACCCCAGATACAAGAAATACCAGCTTGCATGTACCAAGAATGTCTATAATGCATCATCACACAGTACCTCAGGTTTTGCA
AGCACATTCCGGGAAGATAACTCTAGCAACAGCCTCAAGCCCGGGCTTGCCAGGGGGCAGATTAAAAGTGACCCGCCCAGTGAAGAGAATGAGG
AAGAGAGCATCACGCTCTGCCTGTCTGGAGATGACCCTGACGCCAAGGACAGAGCGGGGGATGTCGAGATGGACCGGAAACAGCCCAGCCCTGC
CCCTACCCCCACGGCCCCAGCTGGGGCCGCCTGCCTGGAGAGATCCAGGAGCGTCGCCTCGCCCTCCTGCTTAAGGTCTCTGTTCAGCATAACG
AAAAGTGTGGAGCTGTCTGGCCTGCCCAGTACATCTCAGCAGCACTTTGCCAGGAGTCCAGCCTGCCCTTTTGACAAGGGGATCACTCAGGGTG
ACCTTAAAACTGACTACACCCCTTTCACAGCGAATTATGCACAGCCCCACGTGGCCCAGAAGGACGTGTCCAACTTCACCATGGCGTCGCCCCT
CAGGGGGCCTGGGTTGGAGGCTCTCTGTAAACAGGAGGGAGAGCTGGACCGGAGGAGCGTGATCTTCTCCTCCAGCGCTTGTGACCAAGTGAGC
ACCTCGGTGCATTCTTATTCTGGGGTGAGCAGTTTGGACAAAGACCTCTCTGAGCCGGTGCCAAACCGTCTCTGGGTAGGAGCCAGCCAGTCCC
TCCCCAGCTCGCAGGCCTACTCCCACGGTGGGCTGATGGCCGACCACTTGCCAGGAAGGATGCGGCCCAACACCAGCTGCCCGGTGCCAATCAA
AGTCTGCCCTCGCTCACCCCCCTTGGAGACCAGGACCAGGACTTCCAGCTCCTGCTCTTCCTATTCCTACGCGGAGGACGGGAGCGGGGGCTCA
CCCTGCAGCCTCCCTCTCTGTCAGTTCTCCTCCTCGCCCTCTTCCCAGGGAGCCAGATACCTTCCCACAGAACATCACGAACCAGGCCTGATGG
GAGATGGAATGTACAACCAAGTGCGGCCCCAAATTAAATGTGAGCAGTCTTATGGAACCAACTCCAGTGACGAATCCGGATCGTTCTCGGAAGC
AGACAGTGAGTCGTGTCCTGTGCACGACAGCGGCCACGACGTAAAACTTCCTTTTCCTGTAGATCAAATCACAGATCTTCCAAACAACGATTTC
CAGATGATGATTAAAATGCACAAGCTAACCTCAGAACAGTTAGAGTTTATTCATGATGTCCGACGGCGCAGCAAGAACCGCATCGCGGCCCAGC
GCTGCCGCAAAAGGAAACTGGACTGTATTCAGAATTTAGAATGTGAAATCCGCAAATTGGTGTGTGAGAAAGAGAAACTGTTGTCAGAGAGGAA
TCAACTGAAAGCATGCATGGGGGAACTGTTGGACAACTTCTCCTGCCTTTCCCAGGAAGTTTGCCGAGACATCCAGAGCCCCGAGCAGATCCAG
GCCCTGCATCAGTATTGCCCTGTCCTCAGACCCATGGACTAGCCCACGGCCTCCAGTATTAACCCTCCGCCCTTGGGTGCTGAGCAGAACATTG
CaGCCTCCCAATGCGCAGTGGGGGAAAACGTGCCCTGCTGCTTGGAGCCAGGCGCGGCTCCCCCCGGACCCCCCTGGGCACCCAGCAACACCTC
CGAGAATTGTACCTCTGGGAGGAGACTAGAAGGCACTGACCCGGGAACCTTCTCAGAGAGAGGACCTCCTCTTGAACCCAGGAGCCAAACAGTG
ACCGTGGACTTCTGCCAGGAAATGACTGATAAGTGTACAACTGACGAACAGCCCAGGAAAGATTATACCTAG

TABLE 3
MOUSE NOMENCLATURE
ICSGNM Wnt1
Celera mCG18420
HUMAN NOMENCLATURE
HGNC WNT1
Celera hCG4T496
MOUSE SEQUENCE - GENOMIC
TGAATGCTTGAGATAACCCAAATGCATGCTCCACGCTCGCGCCTTTGCTGCACAAGAGATGAGGGAGGCCTGAGAGGATCCCAAAGTGAGCCCA
ACTTCAGGATAGCGGGGGGGGGGGGGGGTCCTGCTTTCTCCAAGGACGTTCCCACACAAATGTGAATACGCATATGGGCATAAACAAAAAGCTA
CAAAGTGAACCTTTAATTTTTTTTAATGTGTGAACAAAACAACAACAGCAAACAAAACAAAACAAAACAAAAAAAAAAAAAAGAAAGGCCTCCC
CAAAACAAAGAAGACCCAGCAGATGAAAGAAAGTGTTTGCTGACTGGGGCGTGGTGGCACACCCCTTTAATCCCAGCACTTGGGAGGNNNNNNN
NNNNNNNNNNNNNTGGGAGCCGAGGCCAGAGAGTGGTTCCAAGAGCCTGACCTCGGACCCTTCTAATCTAGCTTGCCTGGTATTGCCAGCACGA
GGAGGCAACACCCCTCACAGGTGGTGGGGAACCCCCTTCTAATCCCTCCCTCTAACCCTGTAAGGGCGGGCAGATCAGTTGTGCGGCCCACAGA
GAGGGAGCGGTTGGTGAGGAGGGATAGAGGGTCTGAGAGAACCGGTCTCAAATGGGTATCTGAGTGTCCCAGGACCCTGCGTCGTGTGTGAGCT
GGCTCCCCCCCCGCCCCTGCCAGACCGCAGCCCCAAGCACAGCATGACTGGCCCTGCGCACCCAAGTCACATAGATCCAGAGCCGACCCCCCAA
GGAAACAGAAGCACAGGGCTGGTACCCACAGCCACAACCACCTCTTCATTGCTGGTGGCTAGCAGCAAGGCTGGCCAACCAGCCCCTGGTCTAC
ATTCTTGCAAGTCTTGCCTTGTGTTGAAGAGGCATCCCTGGCTAGGTGTCTAGTACAAAGGAAGTTAGGTATCTGGGCTTACTATTGAGTGCTT
CCTTGATGAGGGTGCTGGAGCCCTAAGCTAGACCACAGCGGGAGGTGCTCTTTCTGACTCCCCCACCTCTGGAGTAGGGAAAAGGCAGCCCAAG
AGCTCCTTCCCATTTAGGGGAAGAAGGCCAAACCATGTATAACTTCTGAGATCGGCCAGCACTGTCTCAGTAACTTCTACCCTACTCCAGAACC
CAGGATCCTCACCAGTGCCCCATCCTCTCTCCTGCCTGTCAGCCTGGGACTCACCCAGGCCCTGGAAGGGTATTTAGCAGAPCCTTCATCAGTA
TCTCGAGGATATTCACGTTTCTCCCGTGGCTGCAGTTCCATTCACACGTTGGATTTTGTCTTGTTCCTATTTGTCCCTAATGCTATCTTTACCT
CCTAGTTCTATCATTCAGTCAGATGTCAAGAAGCGACTAGGGCAAGCTGGGCCACGTAGAGCTGTGTGTGTGTCTGTGTGTGTGTGTGTGTGTG
TGTGTGTGTGTGCGCGCGCGCGCCATGCTTTTACTCCCAACACTTGGGAGGCAGAGGCAGGTGGATCTCCTTGAATTCAAGGCCAGCCTGGTCT
ACTATGTACTGAGTTCTGGGACAGCCAGGCTATATAAAGAGACCCTGTCTCTAAAACCGATGATGATGATGATGACGACGACAGCGGCAACGAC
AATAATAATAATAACGACGAAGGTGATGATGATGATGAAGGGGCTAAGGAAGATTATAGCGCCCAGAGGTTCAACTTCTTAGTGGTCTGGGGGC
TCCCCAAGTTGAACTGTCCCAAACCTAACAACTCATACCGCTGTTCTCTAACCTCTCTGAAGTCTTGCTCTTTCATAAGAGGCCACAGGCAGGC
AGTGGGTGGCCTGAGCTGGCCGAGGGTGCCCTCTTGCCGACCGTCTGGCCTGAGCACTGACCCACACGCAGCTAGAGGCCGGTGGGACAGGCGG
GGCCTGGAGAGGCCTGCGCGTCCACACCCATTGCCTAATACCCCAAGCACGCAGCCAGCTCCAAGAAAGAGGAGGGGCAGGAACCAGAGAGACC
TTGACAAGGGTTGAGACCTACATGAGAGGGGGAAAAAAGGCGCCCTCCCATCACTGCAGGCAGGAAATGAGAGCAACCTGGCCCATCTTCCTTT
TGATGAGAAAAGTGGAAGGCCCCTCCGGCCCTTGCGGGGATGGGGAGGGGGGACGTGTAGCAAAACACTGTGGGAAGAACAACGAGGCCCTGAA
GGGCGGGGAGTGTCAAATAAAAGAGTGGGGACCTCTGGAGATGGGGAAATCAAGCAACTGAGGCGGTGGCTAAAGCCGGGCTACACTCGGGAAT
CTCCTCCTCCTCCCCCTCCCCTGATCTTTGCCTTGGCTGCGGTCTTCGGTGACTCATCTGTTGCTAAGCCGCTGATAGGGGCGGCCAGCCCAGG
GAACCCAAATTATAGCCCCTGGAGGGATGGATGCCCCTGCGGCGGTGAGCTCAGCTGTACGCCTCCCTCCACCTCACCGGTCTTCCACTGCAGC
ACCGAAGGCCACCACCCAGAGGCGCCAGAGATGTTGCAGACGAACTACTGGACTCCAGAGAAGCCCCCACATTCAGTAGTAGCCCCAAGGAGTC
TTCTGACTCACCCAAGACAGAATGTTACCCCCTTGTCTACTATAGGTTGATGGATGGGTTACACCCTCCCAGCCCGAGGGGGTAGCGCCTGCCT
GGGCTTCAAAGGCTTGCCCAAGAACTCAGAGCTCTGCTCCAATGTTCTCTCTCTCTCTCTCTCTCTCTCTCTCTCTCTATACACACACACACAC
ACACACACACACACACGTCTAAGCAAAGCTTTCACCCTCCCCCACCGCCTCCCTCCTAGATCTATTCCAAGCCCTGTGTTTTGCTACAGAGTTT
CTACTTCAATTCTTTAGTTAACTGTAGCCAAAAGTGACAGCAAGCCGCCCCCTGTCCCATATACCTAGAATAAGGAAGTTGAGGGAGGGAGATC
AAGAATTCCAGGAGAGCAGATCTACAGATCAGCATCGAAGAGCTCTTGTTGCTTTTGTAGAAGACTTAGGTTCAATTCCCCAGCGCCCACAAGG
TGGCTCACAACCATTCATAACCTTGGTTCCTTGGGAATCAATACGTATGAGTGTGGTGCGCATATATACGAGCAGGCAAAACATTGAACTACAA
AGAAAAACAAATCTAATAAATAATNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNTTGAGCCACATGTAACCCAGGCTAGCTTCAAACTCAATATG
CAGTTAAATATCACATTGAACTCTGACCTCTGCTTCTCAAGTGATAGGACTATACATGTCACTGAACATGGCTTTATCTTCTCTTTATTTTTAC
CATACCTATGCTAAAGCTTAGGACCTCACATATGCTAGGTAAGTTGTGTGTGTGTGCGTGTGTGCGTGTGTGTGTCCATGTACACATACGTGTG
TGAGCACATGCAAAGGCCAGAGGTGGGGATGGCTGTCTTCCTCTATTTCTCTTTGCCCTATATATTTTTTAGTTTTCATCTTATTTTCATTTTT
AATTTATTTATTATTAGTGTGAGGGGTTGTTGAGTGTGCATCGTGGTGACAGCCATTCTCTCCCCTCTGTCCCCACTTCAGCAGCGGTACTCAT
AGATAATAGGGTTTCCCTGTCTTAGATTGCATTCCACCACCAGAGATCAAGAAAGGTACATGTGTGAAAGGTCTCAGTCAGGGTGTTACTGCTG
TGAACAGACACCATTACCAAGGCAAACTCTTTTTTTTTTTTTTTTTTTTTTTTTTTTTTTTGGTTTTTCGAGACAGGGTTTCTCTGTATAGCCC
TAGCTGTCCTGGAACTCACTTTGTAGACCAGGCTGGCCTCGAACTCACAAATCCACCTGCCTCTGCCTCCCTAGTCCTGGGATTAAAGGCGTGC
GCCACCACACCCGGCTCCAAGGCAAACTCTTATAAGGACAACATTTTTTAAAAGATTTATTTATTTATTTTATGTATGTGAGTACACTGTCCCT
GTCTTCAGACGCATCAGAAGAGGGCATCAGATCGCATTACAGATGGTTGTGAGCCACCATGTGGTTGCTGGGATTTCAACTCAGGACCCCTGGA
AGAGCAGTCAGTGCTCTTAACCACGAAGCCATCTCTCCAGCCCAAAGCACAACATTTAATTGCGCCTGGCTTACAGGTTCAGAGGTTCAGTCCA
TTATCATCAAGGCAGGAACATGTTAGGCATGCTTCAGGAGGAGCTGAGAGTTCTACATCTTGTTCCGAAGGCAAACAGGAGAAGGCTGGCTTCC
AGGGAGCTAGGATGAGGGTCTTAAAGCCCACAGGCACAAGGGCACACTTCCTCCAACAAGGCCACACCCACTCCAACAAGGCCACACCCCCTAA
TAGCCGGTACTCCTTGGACCAGCATATTCAAATCATCACAGTGACCCAGGCCTCAGTGATGACGAATTTCTCAGGGACAAAGTCAGCCGACCCA
TCTATAGTGCTCATTCCCTTGAGCCTTCTGTGCAGGGTACTATGTGCAGAAATTCCTAGCTTACGGACATGTGTAGGAAGCTAGGCCATTCCAT
ATTATACCTCAGAAATTCATCTTTGGAGGAAGCAGAGTTCTAGGCTAAGAGAGAGCCTGGGAAAGACATTTCCATTAACAATAAAATAAAATAA
AATAAAATAAAATAAAATAAAATAAAATAAAATATCTCAGCAGCTAGAGGCATGGCTCATCGGTTAAGAGCATCGGCTGCTCTTCCAGAGCACC
TGGGTTCACTTCCCAGCAACCACATGGCAGCTCACAGCTATCTATAACTCCAGTTCCAAAGGATCCAGCACTCTCACACAGATATGCATGTAAG
CAAAACACCAATGCACATAAAATGAAAATAAATTTATCATTTTTAAGACTCCACCAAGCTTCATTTGGGTTGATGTACATCTTGTTCTAAACAC
ATTCACACCTATTGGGATGACTCAGAAAGTTATGGGACCTGTGACCAAATCTATCGACTCGAGTTCCATCCATGGAACCTTTGTACTAAAAAGA
GAGAATCAATTCTTGTAAGTTGTTCTCTGATTATGCATGCTATAGCAGTCTCACTCATTTGCTTATTCTCTCTCTCTCTCTCTCTCTCTCTCTC
TCTCTCTCTCTCTCTCTCTCTCTCCACACACACACAGACACTCAGGCACACACACACAAAGTGATTAAATGTTTAAATTAAGGGCTGGAGAGAT
GATTCAGCTGTTGAGAGAGCACTGACTGCTCTTCCAGGGTCCTAAGTTCAATTCCCAGCAACCACATGGTGGCTCACAACCATCTATAGTGGGA
TCCGATGCCCTCTTCTGGTGTGTCTACAGTACACATATACATAAAATAAACAAATAAATCTTTAAACATTTTAAATTAAACACAAGTCCCCCAG
GAACAGTAACTAAATCTATCAGTCATTTCTTCCTCAGAGTATAGCTTGCCGGGAAAGGCTGATCCATTCTCAGAGTCAAGCTCTTTTAGAGTCA
GACTCAGGGAGTTTAGTCAGAAATCATACTTAAAAAGCAGACGAGATGCAAACAAAAGGTGCCAAAAAGGTGGCAGGCCAGACCTAAAAGTCAC
TTTCTTTCTGTGTCAACTCTGAAAAGGGACCCCAGCCTGCTCTGCTGCATATCAGGCAGCCACTGAGCCACTGACGATGCAGAAAGAGTGTGCA
TAGGCTTTGAGTTCAAAGGGGCTGTCAGGTCTGGGGGAGGGAACATGCCCCTAATCTCCACTGGAGGACTGGAGGAAGAGAGGTCAGAAGTTCA
AGGCTAGCTAAGACCGTATCTTTAATTTGAAGCCAGCCTGAGCTACATAAGACCTTATAGCAACAAATACGCAAACAAAAATCAAAACCAACCA
GGCTAGAAAGATGGCTCAACACTTAAGAGCGCCTGCAGAGGGGTGGGGGTTTAATTCCCAGCACACACATAGTGGCTCAGGGAATCTGAAGCCC
TCTTCTGGCCACTGCGTGAACTGCATGCGCATGATACATGTGCATACATGCACACAAAATACTCAAAACACATTAAAAAAATAAGTCTAGCGGT
GGTGGCACACGCTAGCACTCAGGAGGCAGAAGCAGGCGGATGTCTGAGTTTGAGGCCAGTCTGGTCTACAATCCAAGTTCCAGGACAGCCAGGG
CTACTCTATCTCAGAAAACCAAATAATCAAACCAATAAGACTCCTCTACCCCCAAAAAACAAAAAAGCAAAAAATAACAACTGGGTTTGGGTTT
AGCTTTGAGATAAGATTTTACTATGTATCCCAGGCTGGCTATACTAGAACTGGTTACTATGTATCCCAGGCTGCCTATACTAGAACTGGTTACT
ATGTATCCCAGGCTGGCTATACTAGAACTGGTTACTGTGTATCCCAGGCTGGCTATACTAGAACTGGTTACTGTGTATCCCAGGCTGGCTATAC
TAGAACTGGTTACTATGTATCCCAGGCTGGCTATACTAGAACTGGTTACGTAGACCAAGATAGCCTCAGACTCACAGATGTGACTCACTTGCTT
CTTAACTCCCAAGTGCTGGATTTACGACCGGTGCCACCAAGCCTGGTGTCCCCAAGGCTTTTTAATCCTCCTGCCTCAGCCTTCCAGGTTCTGG
GACTGGCAAGCATGTGCCCCCACACAGAGTTGGCCTGGGCATTGTTTATTCTATTTTTCATTAGTTATTATTTTGTTGTTGTTTGAGATGGGAT
CTTTCTACATATCTCAGCGTTGTCTCATGTTTGGAGCAATCTTCTTATATTTTATTGTATGTGTGCCTGCATGTGTGTACATGCGTGTGCGCTC
ATGTGCATGTGCGTATGTGTGTGTGTATGTCTGTGTGTGCATCTGTGTGTGTGTGTCTGTGTGTATTGTATGTGAGCAAGTGTGCAAGCTCATG
GAGATCAAAAGACAAGTGTTTAGAATCAGGTCTCTGCTTTCATCACATGGGCCCCAGGGATGAGGCGCAGCTCACCTGGCTTGCCAGCAAACAC
TTGTTTGTTTGTTTGGTTGGTTGGTTGGTTGGTTGGTTTTTGGTTTTTTCGAGACAGGGCTTTCTCTGTATTGCCTGGCAGTCCTGGGCTCACT
CTGTAGACCAGGCTGGCCTCGAACTCATAAATCCGCCTGCCTCTGCCTCCCAAGTGCTGGGATTAAAGGCGTGTGCCACCACGCCCAGCTCAGC
AAACACTTTCTTTCATCTGCTGGGTCTTCTTTGTTTTGGGGAGGCCTTTCTTTTTTTTTTTTGTTTTGTTTTGTTTTGTTTGCTGTTGTTGTTT
TGTTCACACATTAAAAAAAATTAAAGGTTCACTTTGTAGCTTTTTGTTTATGCCCATATGCGTATTCACATTTGTGTGGGAACGTCCTTCGAGA
AAGCAGGAGCAGGAGTTACAGACAGTTATAAGCTGCCTGACCTGGGTGCTGGGAAACACCTCAGGTCCTCTGGAAGAGCAGTAAGTCCCCTTAA
CCAATGAACCATCTATCCGTCCAGCCTACATTTAATTTGTTTTCTTATTTACTTTGTCTGCATGTGCATGTGTGTGCATGGGACACTGGATGCC
TCTGGAGGTCAGAGGTCAGTCTTCTCTCCTTCCATCCAGCGACGCTCGGGGACTCATATCCTCAGGCTTGGCAGCAAGTGCTTCCTTGTCCCCT
TAGTCAGGGATTCTCCATGTAGCCCTGGCTGCCCTGCAACTCTCAATGTAGACCAGGCTAGCCTCGAACTCACAGAGATCCATCTGCCTCTGCC
TTCCAAGTGTTAGGGTTGAAGGCTTGCACCACCACAGGCCTTGCTTTGGGTTTTTGAGAGTGTCTACAGAATGCTGGGAGGTGAGGAATAGCCT
TGAACTTCTGATCTTCCTCCCTCCCAAGTGCTGGGGTTACAGTCATGCGCCACCAATTCCTGGCTTTTGCAGTGCTGGTGATCCAAACTAGGGC
TTCATGCAAGCGAGGCAAACACGGTATCCCAGTTCCTGGGAATGTGGCTTTAACATGGATTCATTTATTAGCAGGGACCTGGGACCTGAAGACA
CTGCCTTACCAAGCATCTCCTCTGTGAAATAGAAGAGAGGAGAAATATTAAGATCCTGGGAAGGGGGCGCTTCCCTGTGTGACAGTGGTGCGTG
ATAACCAGGCTGGCAGTGCCCTCTGCATCCCACATTGGGAACAGCAGCCTGATACTCCAAGGCTGCCATGACCCTGAAGATCACTGGAATCAGG
AGGAAAGTAGAATCCACAATGGAAGAGTTAAGGCTCTTGGGTAAGAGTGGGAGAAAATGGGGGTGGGGGGTGGGGACACTGCAGAAACCTGGGA
GAAAAAAAATCCAACTAAAATCAGGAAACACATGGAGGCACAAATTTGCTAATCTTTCTTTAAAAAACTGTTTCAGGAGATTTATTTTATGTAT
GTATGTATGTATGTATGTATGTATGTATGTATGTATGTATGTATACGTGCACCTGTGTGTGCTTGGTGTCAGTGGGGCTCAGACATCACCTGAT
TCCCTGGAACTGGAGTTACAGGTGGCTATAAGCCACCACTTGGGTGCTGAGAACAGACTCCGGGGCCTCTGGCAGAGCAGTCAGTGCTTTTAGC
CACTGAGCCATCTCTCATTCCCCCCAATTATGTTCATCTTGAGTTGGGCAGGTACGGTGGCGGAATAGGCCTGTAATCCCAGCAGTCACTGGAC
CATCATGGGTTCTACATATTAAACCTTTATGTTAGGTAGGGTCACACAGCAAGATCCGGTCACAAAACCAGCAACAACAAAACCAAAAGGAGCC
AGCTTCTTCCCACAAGCATTCTTTCCCTCAGGTCTTCAGCTCCATCTGACAGCTACTCGGCTGGTGGTCCTATCCTTTCTGAGCCTAGTTGCCA
GAGAAACAAGCCCGGTTCATCTTCATGACTAGCACATCTAATGATAAGCACAGGTTGACTCAAGGTGCCATAGGGTGACACTAGGTACCCAGAG
CGACAGAATGACACCTATGAGTGCACGTCGTTAATCACAAACACACACACACACACACACACACACACACACACACACTCATGCACCCACCTGC
AAACACAATTGCAGCCTTCTGGACGTCTCCTGTCACAGCCCCACCTCCTTCCTGATACACTGCGTTAAGTGGTGACTGTAACAAAATGACTTCA
TGCTCTCCCTGTCCTGAGCCAAATTACACAATTATTTGGAAAGGGCTCAAAATGTTCTTCGTTAGAAGTTTCTGGATACACCAATACACAGGAG
CGTGCACCCTCAGAACACATGTACACTTTGACTTAATCTCACGGGTGACACACCGACGCTTACACTCCCCCTAGCCCACAGAGGCAAACTGCTG
GGCGCTTCTGAGTTTCTCACTGCCACCAGCTCGGTTTGCTCAGCCTACCCCTACACCCCACGCCCGGGAATCCCTGACCACAGCTCCACCCATG
CTCTGTCTCCTTCTTTTCCTTCTCTGTCCAGCCGTCGGGGTTCCTGGGTGAGGAAGTGTCTCCACGGAGTCGCTGGCTAGAACCACAACTTTCA
TCCTGCCATTCAGAATAGGGAAGAGAAGAGACCACAGCGTAGGGGGGACAGAGGAGACGGACTTCGAGAGGACAGCCCCACCGGCGCGTGTGGG
GGAGGCAATCCAGGCTGCAAACAGGTTGTCCCCAGCGCATTGTCTCCGCGCCCCCTGGCGGATGCTGGTCCCCGACGGGCTCCGGACGCGCAGA
AGAGTGAGGCCGGCGCGCGTGGGAGGCCATCCCAAGGGGAGGGGTCGGCGGCCAGTGCAGACCTGGAGGCGGGGGCCACCAGGCAGGGGGCGGG
GGTGAGCCCCGACGGTTAGCCTGTCAGCTCTTTGCTAGACCGGCAAGAGCCACAGCTTCGCTCGCCACTCATTGTCTGTCGCCCTGACCAGGTG
CGCCCTGGTGCTTTTAGTGCCGCCCGGGCCCGGAGGGGCAGCCTCTTCTCACTGCAGTCAGCGCCGCAACTATAAGAGGCCTATAAGAGGCGGT
GCCTCCCGCAGTGGCTGCTTCAGCCCAGCAGCCAGGACAGCGAACCATGCTGCCTGCGGCCCGCCTCCAGACTTATTAGAGCCAGCCTGGGAAC
TCGCATCACTGCCCTCACCGCTGTGTCCAGTCCCACCGTCGCGGACAGCAACCACAGTCGTCAGAACCGCAGCACAGAACCAGCAAGGCCAGGC
AGGCCATGGGGCTCTGGGCGCTGCTGCCCAGCTGGGTTTCTACTACGTTGCTACTGGCACTGACCGCTCTGCCCGCAGCCCTGGCTGCCAACAG
TAGTGGCCGATGGTGGTAAGTGAGCTAGTACGGGGTCCGCCACTTGTCCTGGGGCAAAGAGCCAGGCACGGGCCTTACCCAGCTCCCACGCTGT
GGGGATCACCAACCTACAGACCCCCCTCGTGCATTGTGACTTCACATCCAGGGTGCTCACACCTAGAACTAGCTCTGCTGAAGTGGGGGCACAT
CATTGGCATGCAGAAGCCCAGATACACCAGGCTCAGAGACCATTCCCATTTAATACGACCCCGTTTCTGCTGAGCAACAGGTCCCAACCTCGCT
GTGGTGGGTGCTCAGGTGTCCCTTAGGTCTTGAACCAAAAAAAAAAAAAAAAAAAAAAAAAAAAAAAAAAAAACCAGATATTAGCTTTGAGGTG
AGGGAGTGGAATTCCTAAGTTTTTCAAGGTGGGCAAGGCTGCAGGTGGGGTTTCTCCTCGGGGGCTGACTTGAAGAAAGGAAGAGCTAAGGTAG
CCATGCCTTTTCTGTCCACTCACTAGACTCTGGAGCTCAGGGCCAGGCAAGGATAGGGTGGTACAGCCTGTATGGTTAGGATGCAGGTCCCCTC
CCCTGGACTGAACCCTTATGCATCCCGCCAGGGGCATCGTGAACATAGCCTCCTCCACGAACCTGTTGACGGATTCCAAGAGTCTGCAGCTGGT
GCTCGAGCCCAGTCTGCAGCTGCTGAGCCGCAAGCAGCGGCGACTGATCCGACAGAACCCGGGGATCCTGCACAGCGTGAGTGGAGGGCTCCAG
AGCGCTGTGCGAGAGTGCAAATGGCAATTCCGAAACCGCCGCTGGAACTGCCCCACTGCTCCGGGGCCCCACCTCTTCGGCAAGATCGTCAACC
GAGGTGGGTGCCCAGGAAAGCGACGCTTCCGGGATTAAGGGAAAAGCAGGGTCATCTCCAGGGCATAGGCGGGCGAAGGCAGGGAAGACATCCC
AGGGTTATATGTGATCAAACTGAGAATCGCCTGGTGCCGGCAGTTACCGTAGGTCAGCACCAGATTCTTTCTAGCCTTGCGTTGTGAGCATGAT
CTTTAACGTTGCTGAGCCACTGGCCCACAGAAAGGGAATTCCGGATCGTGGGCGCTGGGCGACAGCTGTTTTTCCCTAGCCTTCCTCAAAGGTA
CCTGGGAAGCTGATCTCTGAGGGCTAGCTAGGGTTGTGCTTCGCACCCAGCAAAGTTTGCACTGCCAATACTAGTAGCGATCTTGGCTATGCAG
ATTTGTTCTACTTGGGAATCTCCCCTTGGAGCTGCTCTGCTAAGGCTCTGGAGTCTCAGTAAAGCTTAGAGAGGAGGGCATTCCATGCTTCGCA
CACATGACTCCAAGGATGTTGGACTGTAGGGTACCAAGTCTTCCAAACAGGGTGCTGAGTTGGCCCCACGCCTTCTCTCAACTGATGCGGGGTC
GCTTCACCCACAGGCTGCCGAGAAACAGCGTTCATCTTCGCAATCACCTCCGCCGGGGTCACACATTCCGTGGCGCGCTCCTGCTCCGAAGGCT
CCATCGAGTCCTGCACCTGCGACTACCGGCGGCGCGGCCCTGGGGGCCCCGACTGGCACTGGGGGGGCTGCAGTGACAACATCGATTTTGGTCG
CCTCTTTGGCCGAGAGTTCGTGGACTCCGGGGAGAAGGGGCGGGACCTACGCTTCCTCATGAACCTTCACAACAACGAGGCAGGGCGAACGGTA
CGTCGGTGTGTCCGGAACCAATGGCAGGGGAGATGTAAGACAGGTGCACGGGGACAGAGGCACAGGGAAGGGGCTTCCCGAGAGAGTGGGACTC
TAGGAGGGAAGACAGAGAAGAGGTGGTGGTTGAGGGCAAAGAGGTTCCTGAGCTGATGACAGAACAGAAGAGATTAGCAGGCTATCAACACGTG
GGATGTATTGAGATGGCTCCATGGCACACTTTTGAAAGATAAAAGTGACTTGCTGGCGTGGAGCAGAGTCTGGCCGAATGTCCCTATCTCAGCG
GGCCATTTTGCACTTCCTCTCTCCCGAGCTTAGTCACACCTGGACCTTGGCTGAAGTTTCCACAGCATCGACGTGACCCGGGTGGGGTGGGGGT
GGGGAAGTATGGGTGGTGGTTCGTGGGATGTTGGCTTTGACCTTTTCTTCCCTCCTCCCCTCGTCCCCTCCTCCCCCAGACCGTGTTCTCTGAG
ATGCGCCAAGAGTGCAAATGCCACGGGATGTCCGGCTCCTGCACGGTGCGCACGTGTTGGATGCGGCTGCCCACGCTGCGCGCTGTGGGCGACG
TGCTGCGCGACCGCTTCGACGGCGCCTCCCGCGTCCTTTACGGCAACCGAGGCAGCAACCGCGCCTCGCGGGCGGAGCTGCTGCGCCTGGAGCC
CGAAGACCCCGCGCACAAGCCTCCCTCCCCTCACGACCTCGTCTACTTCGAGAAATCGCCCAACTTCTGCACGTACAGTGGCCGCCTGGGCACA
GCTGGCACAGCTGGACGAGCTTGCAACAGCTCGTCTCCCGCGCTGGACGGCTGTGAGCTGCTGTGCTGTGGCCGAGGCCACCGCACGCGCACGC
AGCGCGTCACGGAGCGCTGCAACTACACCTTCCACTGGTGCTGCCACGTCAGCTGCCGCAACTGCACGCACACGCGCGTTCTGCACGAGTGTCT
ATGAGGTGCCGCGCCTCCGGGAACGGGAACGCTCTCTTCCAGTTCTCAGACACACTCGCTGGTCCTGATGTTTGCCCACCCTACCGCGTCCAGC
CACAGTCCCAGGGTTCATAGCGATCCATCTCTCCCACCTCCTACCTGGGGACTCCTGAAACCACTTGCCTGAGTCGGCTCGAACCCTTTTGCCA
TCCTGAGGGCCCTGACCCAGCCTACCTCCCTCCCTCTTTGAGGGAGACTCCTTTTGCACTGCCCCCCAATTTGGCCAGAGGGTGAGAGAAAGAT
TCTTCTTCTGGGGTGGGGGTGGGGAGGTCAACTCTTGAAGGTGTTGCGGTTCCTGATGTATTTTGCGCTGTGACCTCTTTGGGTATTATCACCT
TTCCTTGTCTCTCGGGTCCCTATAGGTCCCTTGAGTTCTCTAACCAGCACCTCTGGGCTTCAAGGCCTTTCCCCTCCCACCTGTAGCTGAAGAG
TTTCCGAGTTGAAAGGGCACGGAAAGCTAAGTCGGAAAGGAGGTTGCTGGACCCAGCAGCAAAACCCTACATTCTCCTTGTCTCTGCCTCGGAG
CCATTGAACAGCTGTGAACCATGCCTCCCTCAGCCTCCTCCCACCCCTTCCTGTCCTGCCTCCTCATCACTGTGTAAATAATTTGCACCGAAAT
GTGGCCGCAGAGCCACGCGTTCGGTTATGTAAATAAAACTATTTATTGTGCTGGGTTCCAGCCTGGGTTGCAGAGACCACCCTCACCCCACCTC
ACTGCTCCTCTGTTCTGCTCGCCAGTCCTTTTGTTATCCGACCTTTTTTCTCTTTTACCCAGCTTCTCATAGGCGCCCTTGCCCACCGGATCAG
TATTTCCTTCCACTGTAGCTATTAGTGGCTCCTCGCCCCCACCAATGTAGTATCTTCCTCTGAGGAATAAAATATCTATTTTTATCAACGACTC
TGGTCCTTGAATCCAGAACACAGCATGGCTTCCAACGTCCTCTTCCCTTCCAATGGACTTGCTTCTCTTCTCATAGCCAAACAAAAGAGATAGA
GTTGTTGAAGATCTCTTTTCCAGGGCCTGAGCAAGGACCCTGAGATCCTGACCCTTGGATGACCCTAAATGAGACCAACTAGGGATCCAGGTCT
CCTGAGAGAAAAGCCTGCTCAAAAGCTCCTGCAGGGCTCTTCCTCCTGTTCTGGCAGCAGGTGCTTAAGTGATGAGGACAAGTGTGTCACCTGA
TGGGGACTCAGCACACCAACAGTCCAGCTCTCCTAGTGCAAGAGCTGGGGAGGTAGCAGCAATGAAAGGAAAGGATAGCCATGACTAGGTCCTG
AGAGGGGGGGGGGGGAAGCTCAAGCAGAAAGGGTGTCATTGCCCCTGGGAGGCTATCAACCTTTTCTGCAGTCTGAGCTTTGAGCAGTGGAAGG
CCCACCTCTTACACTCTTCCTGGAAATTCTTTATGACCAACCCAACCCTAACCTACAGGTAAGGGACATCCAGGAACAAGCCATGGGTTAGAGC
TCGAAAGATGGCTCAGAGGTTAAGAGCACTGGCTGCTCTTCTAGAAGTCCTGAGTTCAATTCCCAGCAACCACATGGTGACTCACAACCATCTG
TTAATGGGATCTGGTGCACTCTTCTGGTGTGTCTGAAGACAGCTATGGTGTGCTCATATAAATAATAATAAAGAAATGATATTAAAGAAAAGCC
ATGGGTTAACAGTGGGCCCTAAACTTGAACTAGAAAACTTAAAGATGCTCATAGGGAAGAAGAAAAGAGCAGAAAGCTTAGCTTCTAGACAGGG
GTAAGGCTTAGAGCTCAATAAAAAAGGAACCCCAAGAGTGATTGGTCCACAACCAAAGCAGTAAGCTAACCACACCCACAAAGAGCCAGAATGA
GGATTCCTTATGACCTGCAGGCTACCTCTGTGCTTGCAGTCACCAGTCAGCAGGAAAGGAATCCTTCTCAGAGTACTCAACCTGACGCCAGTCA
TTTCTTCCTCCCTTCCTTGTCCAACCAGAGCCTTAGCCCTGACTCAGGGAACACTAACCCTTTCCAATACCTGACCTTCATCATAGCCCCTTCC
CACCAGAGAGGACCCGAAAGGCATTGACAGAAGCTACAGAGGACCCCCGCCTTCCCAAGAGCCATAACCAGTCTCAATCAGCTCACAGAGTTTC
TTCTTCTTCCCAGGATCCTTTGCTGCAGGCTATCACCTCTGGGGAAGGGGGGGGGGAAGAGGTGGGGGGGGAGGGGTGAACTCTGACCCCTGTT
GGTTTTTTGACTCTGTGAGTGGGTAGAGAAGTAGATTCAGGGCCTGCTCAAGCCATTTCCTTGCCTTTCTCTCCACTGGCAAAGCTAGCACTGG
GTCAGTTCTGATGAGTGGATCCAACCTGGGATACTGGATGTCTGGATCTCTAAAAGGTTCTGGGTCTCCAGTGATGATCATCCAGAAGAGCCTA
CTCAGTCCTAACCCAAGCTGAGCTATCAGCCCCTCAGCGCTCCCAAAGTATGAAGCCATTGGGCTGTCCCAGGGCACAAATGTGTGTGTGTCTG
TGTGTGTGTCTGTGTGTGCATGCCTGCACATGCTGTGTTGCACGTGTGTGCCTGTGTGTATGTGTGCATGCACGCATGTGCACATGCACGCTTG
TGTGTGTGTGTATGTGTGTATGTGTGTGTGCATGTGCACGTGTGTGCACGCATGTGCGAGAGTGTGTACGCGTGGGCTGCTGTAACTCAATGTA
TCAGGGAGAAGCAGCAGAATGGAGGAACCAAACCATCGCCGACCAATCTCACCCAAATCCTAACCCAGGAACCTGGAAACTAGTGTGCTGAGCA
GAGGTGTCTTTCAGGAGTTGGAAAGAGAGCACCACCACCCCCTTTTGCCCAGGTATTTGACACATCTTTTGTCTTCAGTCTTCTCAGTCCCATT
TGCCTTGCTTGACCCTTAATTGAGAATATTCAAATATCCATCTTTGTTGTCGCTTAAGGAGGGGATAGGCTTCAAATGACATAGTGTCTGTGAC
AGCTTGGGTTGTGTGGACGGCTACTGATACCTTTTCTGTACTAGCCTCTGTCAAATGCTGGCAGTGCAGCAATCTACAAGCCAGAGGTCCAGCC
AGGACTCAACCAGGCCAACAAACACTAACAGGCATAGAGGGAAAGACCCTCGGTGTGCAGGAAAGGCTTCAGCACCCTTCTCTTCCAGGGGCTC
GAACTTGGAAGAAGTGTGCCATGGGAAAATGCAGAACAAAGCCAGGGCCCAACACAACATGCACAAATAAAAGGAAACACTTCAGATAAAAGTG
CTGGAAATGTGTGGCGTGATGGTAGAGCACGTGTAAGGACGAGATGGGTTGGATCCCCAGCCGTGTGTGTGTGTGTGTGTGTGTGTGTGTGTGT
GTGTCTGTGTGTGAAGGGGCTGTGGGGTCTACAAGTGTTGCCTAGGAGCTGAGGACTCTTTTGTCCCTATTACCTGTGTTATCTGCGCTGCCCT
CTCAACTGGATCAGTAGATCAGTAAGGAAGGGGGGGGGGAGGTGCGTACCTAGAAAGAGTCTCAGGGCTCAGAACACAAGGCGCTGTCCATAAG
ATAATGAATGTCATCAGAAATATGCCTGACGCACCTTCCTGAGGCTTCCACCCACTCGTGATCTTTAGATGCCATGCAAATACACACCACCCCG
GGGACAGGGACCCTGGTCACAGAGCTGACCTCAGTCAGAGCTGTTGCGGGGACTGAGTCTTGAAATATCCCAGGGGTGGCGGAAAAGCTGAGAG
GACCAAGACAGCCAGCGGGGCTGCACGGTCCCTGGGGGGTCAGCACCGAACACCCTGACACTGGCATCTCGGAAAAACCCTGTCAGTGGTAACT
GCAAATACCAGAGGGTCCAACCCCCCCTTCCTTACGGCCCCAGGCAGGCTCAGTCCCTTGGCTCCAGGGAAGAAAATAAGCTGTCTGGCTGTGG
TGTGAGCAGGAACATCCGTGCAGAGCTGCCCACCAACTCACCCTTACTCAGTCTTTCCACACCATGCCAGGGCCGGGCATGAGACAGACCACCC
CCTTACCCCCTCCCCCGTCTGGGCTAAAAGCAGACACACTGGGTTGGTGAGAGAGCAGCAGGTTATAAATATGCCCAAGGAACTGCCGCCAACT
CGCTCCCCTGTCATCCCCTGGGAGGGGGTAGGGTGGGGTGACACCTAAGGAGGAGCCAGGGCTGCCCTATTCAGTGAGGCCCAGAGAGAAGTGG
AGCCTCCAACCAGCTGCTGAAATATTTATTGATTTTTTTTCCTTTACCTTTCCCAGGTGAATGTGCCACCTTTGACGTGTGCCCTGGACTTTTG
TCTACCCCACCCTCCCTGGTATATGGCCTTATGTCCAGCGTGAAAGGGCTCAAAGCCCTGGGCCATTTCCTTACACCCTCAAGAAAATAGCAGA
CTTTTTAATTCACTCACTGAGTGGACATAGTGAGGTGCGCATGCGCACCTGCAGCCCCGGCGCATGCACACCCTACTCCTGGGTGATCGTACCA
GCTCCAGCCTCTGTTCTGCACGCTGTGCCTTCAACCTGGCAACCTCCCAGGGGAAGGGAAGGGAAGGGAAGAGAAGGGAAGGGAAGGGAAGGGA
AGGGAAGGGAAGGGAAGGGAAGGGAAGGGAAGGGAAGGGAAGGGAAGGGANNNNNNNNNNNNNNNNNNNNNNNNNNNNNNNNNNNNCTAAGGCA
AAAGGGGCAGCCCTCCTCTTCCAGGGCTGAGCCCCTCCCTTCTCCATCCTCCCTATTAATCCTCGGAAGTGAAGGGGCCGTCCCTGGCGGCCCT
TCAGCCCTATTGTGGCCCCTCGCCCTCCTCGGCTGGCGTCTAATTAACTCCTCCTGCCCCTGGTTTTGTGTGCCCCCCTTACTCCACCTTGTCG
GCCCCGCCCCTCCGCTCGGTCCGCTCCCCCCGACGGACATTCCAGTCAGGCCGGGCCAGTGTGCCAGGGCAACCAGGGCCAGACCGAGGCCTAA
TCCCCCCTCCACCCGGCCGGGCTCAGGGAGCCCCACTCACCTCCCGGCTCTCTAGGGGCTGCTCGGCTCTGCTGTGGCGGAGACACACAATCAG
GACAAAGGCGCAGCCCCCAGGGGGCTAACTCAGCCTTCACGCCCAGGTTGTGCGGCATTTGGGGAGTTTAATGAATTGTCCATCACGCCTTTCA
GGGCCCGCCCGTCAGCCTGGATTAATCTTCGGAGCCCTGGTGGGGCAGGAGACACTAAGCCTGTATTTATTACTCTCCCATTGTCACTAATTGA
GGTAATTATCTGTGACTCTGGCCTGGCCAACAATGCTGCATTCACTCCTGGGACCTCGATGGGCCTGGCTCCCACTCTCAGCACCAACCCTCCC
TCCCTTCCTCTCTAAAACGCGCCAAGGGCCAAGATGGGCCCTGGAGCATGTTGGGAAAGTGGACAACGTTCCACAATGTTCCACGTTCACGTAG
TGTCTCCCAAAGCCTCCTCCACTATGGAGCAGTGTTGAGGAGAAACCTGGGTCTCCTAAAGTTGGTGAGAGAAGAGGGCACACAGGCTTGACCC
GCAGAAACCCCAGGCCACAGCCAGCCTGGCATGCTTCATTCCTGGGTAAATGGCACTAAAGTCTCACGGGGCCTTTCACATGTCCCTGTAGCCT
ACAGGACTCTGGAGTGCTAGAGGGCACATACTATTCCCTGCTAGATGAGACCTTCTGGAAGCAGAAGCTACCAGAGTTGCCAGAGCGGCAAAGT
TGGAGAAAGTTAACAAGAACAGGATAGGGCGCAACAGGGAGCAGAAGGGCAGGATGCAGAAGCATCCTAGGAAGAAGCAACCCAAAGTCAGAGC
ATTCCCCAGGGATCACACCAATGTGACCATCAATTAGTGCCATGCAGCAAGCTGGAGAAAAACCCAATTTAGAGTAGATGTCATATGAAGATGG
CCTTCTGGGTCAAGGGAATATTCCTGGAGAACGTGTGTGTGTGTGTGTGTGTGTGTGTGTGTGTGTGTGTGTGTGTGTGTAATGCATGTGCATG
TGGTGTGTGTGTGCATGTGTTGTATATACATGTATGTGTGTGAATTCTTGTGTGTGTGCTTGTGTGATGTGTGCGTGTGTGTGTGTTTAAGATA
CAATAAAGAAGAGTAGACTTGGATGAGACTTGATTTTGAGATTCTTGATGGCAAAAATTATGTTTTGGGTCACTGTTATTTTTATAAGATCCTA
ATACTTTACCTAGAATGCCAGGCTATATGAAGCCCGTACTTCACAACCGTTGCGGCCTGTCAGATCTTCTCTTAAATGTCTGTCCTCCGGCTCT
TCATTTACCTAGTTCTTGGTCAAAAGAGATGTCTAGAGCATTCCTGGACAGTCCTGTCCTGGATGGACAACTGACCAGCTGAAGCACTAAGGAT
CCTGGTAGAACTCAGCGCCAAAGTTCGAACAGCGCCACCTCGTGGCAAATCTTGGGAGAGCTTCTGGAGACACTAGCTAAGAGGATAGAGCCAG
CGGCTGCAGGGGCTCAGGCTGGACAGCAGCCATCCTCCTTCATGGCTGCGTTGTTGTCTTCCCGGCTTTTATTTTAAATTGTCCCTCCATTATT
TGAAGTAATGTATCGTGCACTTCGGGATGTCATCAAGTTTCCCATGTGCTGAAGATAACAGAGAATACCCGGTCCTTTTGCCTGTACCCCCTGA
GTGCTAGGACTTTTACGGGGCTACCATGGCCAGTTTATAGAGTTTTGGGGATCAACTGTGGGACCTTGTGAATATTAGATGAGTTTCTATCAAC
TGAGTTACATTCTCCTGCCTCACTTCCCTCTTCTTACACAAACCTTTTTGGAGGTGTTTTCCATGCCAAGATTTCGTGGCTACAGAGTCAAACA
TGTAAATCAATGTATGTAAATCATATTCTAGTAACAAATTTCAAAGGGTAACCAAGTCAACAAAGATGCCAGGCATGGTGGCACACACCTTTCC
TCAGAGGATTTGGGAAGCAGAGGAGGGTGGATCTCTGGGACTTTCCAGGCAGGTTGGTTTAAATAGAGGGTTCTAGGTGAGTGGGGGCTATGTG
GTGAGACCCCATCTCCGATACAGTCCCACCATCACTAACCCACACCAAACCAACCAACCAAAAAGAGAAAGCACAGCAAAGGTCACGATGGAGA
GCCAGGGAAGTATTCAGAACTGAAGTACAATAAGATACTCCCAGCTCAAAACTCTCTGTCATCACTCTTTTGATTCTTCTCACAAAAGATCCTG
TGTTTCTTTAGCCAGAGGCAGAGCCTCATTGCTTTTCCCATGTTGCCTTGGAATTCCTGGGTTCAAGGGATTCCCCACCCACAACCCGCCATGT
AGCCGGGACTACAGGTGTATGCGTCTGCCCCATGTGAACGTCAGATGCTTGCTTTATGCTTGCCTTCAGTAACCTCTTCGTGACCAAGGCCTCT
TTCTTCCCAGGGCCTTGTTCCCTGTGGAACTCATGTCTTCTCACATTCTGGACGCATCTGTTTCCTCATCATCTTTGTGAGAACTACCTCAAAC
ATCCCTTCTTCAGCTTTCCCTTGGCAGAGTGACCTTGCCTGCCCGAACTCTTGAAATGCTTTGCTGGCCATCCGTGTATGCAGTAGACCAGGCT
TGCACCTGCATGAGGACACAGGTTAGGTGTCTCTCATTTTGCCATCCTTGCTAGCACCAGATAGGGACATTATATGCCCTCATCAGATGATGAA
TAAGTGGATTCATGGATGAAGAAGCAGACGCATGGATAGATGGATATGGAGAGGCACTTGAATGGATGATAGATGAGATGTGGATAGATCAGTA
AGTGCATAGGTAGGATGCATGGATAGATGCATGCATAGGAAGGATAGATAGGTGAGTGGCTGGATGGGTGGATGGATGGAAGGATGACTGGATG
GGTGGATGGGTGATGATTGGATCGATAATTGGGTGGATTGATGGGTGGGTGGATGGATGGATGGATGGATGATTGGATGGTTAATTGGGTGGAT
TGATGGGTGGGTGGAATGGATGGACTGATGGAGCATTTATCCACAGATGACTTGTATATCCTAGTTAATACAAGGAGATTCAAAGAAGGGAGCC
TACCCCCATGGCATGCCATGAAGAGGCAGGAACACCCCTAGTTTAAATTTTTCTCTCTATGTGCACCCTTGAAGACTTGTCTCCCAGTAGGCAT
TTGTGGCTATATCCCATTCCCTATGAGTCCAACTCAAGGAGGTGTCCTCCTCCCACTTCTTTCCATAAAATATGGAAGAGTTCACAAATTTGAA
TGTTGGCCTTGCAGAGGCACTGCGCTAATCTCCTCTCAGCTGCTCCAAATTTAGTGTAAAAGTGATCTCCCCAAAGGTAGAAGGTGTGCCCGAG
TCTCCTCGATCTCCCTGGAACACATGGTTAGTGGGCAAGTGGACAACTGAGTAATTACTGAGAGAATACTACCCTGTCCTCAACGAACACTCTG
TGCACACATCCCAGGACCCTGCATTGAGCGGCTCTCCTCTCTTGCAAACACATCCACATTACTGATCTCTGTTTTGTTTTGTTGTTTTTTGGTT
TTTCTGAGACAGGTTTCTCTGTATAGCTCTGGCTGTCCTGGAACTCACTTTGTAGACCACGCTGGCCTCGAACTCAGAAATCTGCCTGCCTCTG
CCTCCCAAGTGCTGGGATTAAAGGCTGTGCCACCACTGTGCTCACTGATCTCTTTGATGTCCCAGTTATAGCTCTTGGGTTCCCCACCCATTTG
TAGGGGGACCCAGGACACCTCAGAGCTCTCCCAAGTCTAAAAAGGGCAGGGTTCCTGGCTCCCTTAATGCCTTATCAACCACAACAGAACTCAG
GGGCAGAAAATGTTCCCAGGAAGAACTTAGCTGTGGGGAGAGTCATGGGGTCCCCTGCTAACCTAAGATGTGTTGCCCCCAAAAGAGTAGCCCT
CCAGGACCTGCTGTCTTTTCTCCTGATCAAGGGGGCAGTTCCAGGGTGGGGGTGGTTGTGCCCTCCTTCAGAAGGCTGGGTACCTGGCAGACAC
AGCTAGATTTAGAAGTCATTTTCACAGCAGGGAGTCGCTAACCTGCCACCCACAGGGCCCCATGGGGCCTAAGATACTACTTACCAGGGTCTGG
CCCTGAAACCTGCAGACACTCCTCTCCTTCTCTCCCAGAACTTTGGCAGCTGGCTCCGACTCTATCCTTGGCAAGCTCTGGAAGAAGGCACGTG
TTGCCCTGACACCCTAAATACCCAAATGTTCCCCCACACAAAATGGGTGGTTGTTACCTAACCTAGGATATGCCTTTTTCAATGCTGGGGACCA
AACTCAGGACTCTGTACATTCTAGCCACACATGCATACTACCATTGAGCCACCACCACAACCCGGTTCCAAAGGGACTTCTTTTTTTTTTTTGG
AGACAGAATCTTGCTGGGCTGAACTGGGATAGGCTGGAAGTTGCTGTGTAAACCAGGCTGGGCTCAAACTCATGACCATTCTCCTGACTCCAGT
TTTCAAGAAAGTGCGCCATGCCTGGCTTCCTTCTCTCTCTCTCTCTCTCTCTATCTATCTATCTATCTATCTATCTATATATATATATATATAT
GTTTTATTTTGCCTGGATGGCATGCAGCTCATTATGTAGACCAGGCTGGCCTTGAACACCCAGAGACCCACCAGCCTCTGCCTTCTGAGTGCTG
AGATTAATGATGGAGAGTGCCACCAATGCAGAGTGCCACCAACCGGGCTTCTTTCCCTAATTTTTATCTACAAGTCACCTCCTCTGTGAACTCT
CCCTGGATTTTCTAAAACTAGGATCTCCCTTGATTGACTCTTCCTAGAACTTAACACAACCGTCCTCACTGTCAGACAGCCCTTGCCATTTCTG
CCCTTGAGCCCAGCCCAGAGTCTAGTTCCTAGGAAGTGTTCAGTAAATGCTAGGTTTCGCTTGGCAGCAGAAAGGACTGCATTGGACGTGGTAA
GACCTTTGGTATCTTTGATCTTTGCCCAGGGTGACCCGGC
MOUSE SEQUENCE - mRNA
AGTGGCTGCTTCAGCCCACCAGCCAGGACAGCGAACCATGCTGCCTGCGGCCCGCCTCCAGACTTATTAGAGCCAGCCTGGGAACTCGCATCAC
TGCCCTCACCGCPGTGTCCAGTCCCACCGTCGCGGACAGCAACCACAGTCGTCAGAACCGCAGCACAGAACCAGCAAGGCCAGCCAGGCCATGG
GGCTCTGGGCGCTGCTGCCCAGCTGGGTTTCTACTACGTTGCTACTGGCACTGACCGCTCTGCCCGCAGCCCTGGCTGCCAACAGTAGTGGCCG
ATGGTGGGGCATCGTGAACATAGCCTCCTCCACGAACCTGTTGACGGATTCCAAGAGTCTGCAGCTGGTGCTCGAGCCCAGTCTGCAGCTGCTG
AGCCGCAAGCAGCGGCGACTGATCCGACAGAACCCGGGGATCCTGCACAGCGTGAGTGGAGGGCTCCAGAGCGCTGTGCCACAGTGCAAATGGC
AATTCCGAAACCGCCCCTGGAACTGCCCCACTGCTCCGGGGCCCCACCTCGTCGGCAAGATCGTCAACCGAGGCTGCCGAGAAACAGCGTTCAT
CTTCGCAATCACCTCCGCCGGGGTCACACATTCCGTGGCGCGCTCCTGCTCCGAAGGCTCCATCGAGTCCTGCACCTGCGACTACCGGCGGCGC
GGCCCTGGGGGCCCCGACTGGCACTGGGGGGGCTGCAGTGACAACATCGATTTTGGTCGCCTCTTTGGCCGACAGTTCGTGGACTCCGGGGAGA
AGGGGCGGGACCTACGCTTCCTCATGAACCTTCACAACAACGAGGCAGGGCGAACGACCGTGTTCTCTGAGATGCGCCAAGAGTGCAAATGCCA
CGGGATGTCCGGCTCCTGCACGGTGCGCACGTGTTGGATGCGGCTGCCCACGCTGCGCGCTGTGGGCGACGTGCTGCGCGACCGCTTCCACGGC
GCCTCCCGCGTCCTTTACGGCAACCGAGGCAGCAACCGCGCCTCGCGGGCGGAGCTGCTGCGCCTGGAGCCCGAAGACCCCGCGCACAAGCCTC
CCTCCCCTCACGACCTCGTCTACTTCGAGAAATCGCCCAACTTCTGCACGTACAGTGGCCGCCTGGGCACAGCTGGCACAGCTGGACGAGCTTC
CAACAGCTCGTCTCCCGCGCTGGACGGCTGTGAGCTGCTGTGCTGTGGCCGAGGCCACCGCACGCGCACGCAGCGCGTCACGGAGCGCTGCAAC
TGCACCTTCCACTGGTGCTGCCACGTCAGCTGCCGCAACTGCACGCACACGCGCGTTCTGCACGAGTGTCTATGAGGTGCCGCGCCTCCGGGAA
CGGGAACGCTCTCTTCCAGTTCTCAGACACACTCGCTGGTCCTGATGTTTGCCCACCCTACCGCGTCCAGCCACAGTCCCAGGGTTCATAGCGA
TCCATCTCTCCCACCTCCTACCTGGGGACTCCTGAAACCACTTGCCTGAGTCGGCTCGAACCCTTTTGCCATCCTGAGGGCCCTGACCCAGCCT
ACCTCCCTCCCTCTTTGAGGGAGACTCCTTTTGCACTGCCCCCCAATTTGGCCAGAGGGTGAGAGAAAGATTCTTCTTCTGGGGTGGGGGTGGG
GAGGTCAACTCTTGAAGGTGTTGCGGTTCCTGATGTATTTTGCGCTGTGACCTCTTTGGGTATTATCACCTTTCCTTGTCTCTCGGGTCCCTAT
AGGTCCCTTGAGTTCTCTAACCAGCACCTCTGGGCTTCAAGGCCTTTCCCCTCCCACCTGTAGCTGAAGAGTTTCCGAGTTGAAAGGGCACGGA
AAGCTAAGTGGGAAAGGAGGTTGCTGGACCCAGCAGCAAAACCCTACATTCTCCTTGTCTCTGCCTCGGAGCCATTGAACAGCTGTGAACCATG
CCTCCCTCAGCCTCCTCCCACCCCTTCCTGTCCTGCCTCCTCATCACTGTGTAAATAATTTGCACCGAAATGTGGCCGCAGAGCCACGCGTTCG
GTTATGTAAATAAAACTATTTATTGTGCTGGGTTCCAGCCTGGGTTGCAGAGACCACCCTCACCCCACCTCACTGCTCCTCTGTTCTGCTCGCC
AGTCCTTTTGTTATCCGACCTTTTTTCTCTTTTACCCAGCTTCTCATAGGCGCCCTTGCCCACCGGATCAGTATTTCCTTCCACTGTAGCTATT
AGTGGCTCCTCGCCCCCACCAATGTAGTATCTTCCTCTGAGGAATAAAATATCTATTTTTATCAACGA
MOUSE SEQUENCE - CODING
ATGGGGCTCTGGGCGCTGCTGCCCAGCTGGGTTTCTACTACGTTGCTACTGGCACTGACCGCTCTGCCCGCAGCCCTGGCTGCCAACAGTAGTG
GCCGATGGTGGGGCATCGTGAACATAGCCTCCTCCACGAACCTGTTGACGGATTCCAAGAGTCTGCAGCTGGTGCTCGAGCCCAGTCTGCAGCT
GCTGAGCCGCAAGCAGCGGCGACTGATCCGACAGAACCCGGGGATCCTGCACAGCGTGAGTGGAGGGCTCCAGAGCGCTGTGCGAGAGTGCAAA
TGGCAATTCCGAAACCGCCGCTGGAACTGCCCCACTGCTCCGGGGCCCCACCTCTTCGGCAAGATCGTCAACCGAGGCTGCCGAGAAACAGCGT
TCATCTTCGCAATCACCTCCGCCGGGGTCACACATTCCGTGGCGCGCTCCTGCTCCGAAGGCTCCATCGAGTCCTGCACCTGCGACTACCGGCG
GCGCGGCCCTGGGGGCCCCGACTGGCACTGGGGGGGCTGCAGTGACAACATCGATTTTGGTCGCCTCTTTGGCCGAGAGTTCGTGGACTCCGGG
GAGAAGGGGCGGGACCTACGCTTCCTCATGAACCTTCACAACAACGAGGCAGGGCGAACGACCGTGTTCTCTGAGATGCGCCAAGAGTGCAAAT
GCCACGGGATGTCCGGCTCCTGCACGGTGCGCACGTGTTGGATGCGGCTGCCCACGCTGCGCGCTGTGGGCGACGTGCTGCGCGACCGCTTCGA
CGGCGCCTCCCGCGTCCTTTACGGCAACCGAGGCAGCAACCGCGCCTCGCGGGCGGAGCTGCTGCGCCTGGAGCCCGAAGACCCCGCGCACAAG
CCTCCCTCCCCTCACCACCTCGTCTACTTCGAGAAATCGCCCAACTTCTGCACGTACAGTGGCCGCCTGGGCACAGCTGGCACAGCTGGACGAG
CTTGCAACAGCTCGTCTCCCGCGCTGGACGGCTGTGAGCTGCTGTGCTGTGGCCGAGGCCACCGCACGCGCACGCAGCGCGTCACGGAGCGCTG
CAACTGCACCTTCCACTGGTGCTGCCACGTCAGCTGCCGCAACTGCACGCACACGCGCGTTCTGCACGAGTGTCTATGA
HUMAN SEQUENCE - GENOMIC
CCCTGGAGTCGGGAGATGACTAAGAGGGAGTCCCTGAGGTCTTTGAAGATGTGTACAACCGAGTTCTACATCTGAAAGCCTTAGAAATTCCCCC
AACCCTGCAGGGAGCTTGGGTGGGTGAGGTGGGAGCAGAGCCTTTGTGGTTCTGTTTTTTTGTTTTTTTTTTTTTAAATTCACTTCASGGTTAA
TCAGTCTTTTAACCAACCTACCTACCACCATCCTAGCACCCCCAAAGCACTGGAGCTATGGAGCTGCCCTTCCGCTTCCCAAGATGTAGTTTTA
TATTTGCCCTTCAAGACTGCCCTTTTTATGTTTATCCTAACCTGCTATTACCCTATAAAACACTGAGAGGAGGAGAGAGGTTGGGGAGTGATTT
TCCCTAAGGCCCTCTTAAAAGGTTGGCTTCTTTGTTATGTTTTGGGGGTCACAGAAGCTCAGGCTGCCAAGCCTTTCCCAGACTTAAATAAACA
TGCTCACCATTTGGGTGTCCAAGCCTTCCCCTACACCAGGCCCTGTGGGGAAGGAGGGGGACCCTCACTTCATTTTAGGCAGGGGCTCTCCGGA
GCCCTGAGAAGACGAGAGACAGCACCCGCTTAGAGACCAGGCCTCTGCTACCCCAGGATCTAGGGCTCCTGGGCCAGCCTTCTGATCCCCCTAA
CTCCAACCCTCCAGGGGTGAAGCTGTTTGCACAAGTCTTGGGAATTCCCCTGCAGATGGCAGCTGCCTATTAACCCCATAGTCCCCAGAGCACT
GCCCCCATCTCTGGGCTGAGCCCCCATGGGTCTTTACACCTCAGGGAGTGAACTCCAGTGGGAAACCAGGAGAGGCAGCCATGCCTCCCAGACC
AGGCCAGCAGTGACCTGTTCTCTGCCTCTCCAGCCGTTCTTCACCTGCTCAGGTGAGCAACACCTGGAGCCCCCACAGCACAGAGGAGGGGACT
GGCTCCCCAGCTTCAAAGTCATCAGGGTCTTTGTTCCCAGCTTTGGCTCCTGGGAACCCCAGCAATTAGGGGAGCAGATTTAACCCTTAAACGG
TTGGGGGCGTCACCCCACCCCCGCTGAGACACTGGGGACACCAGCCTGGGCTTGTCCAGCCAAGATGGGGGGAGAGAAAATGGTGGAGGGAATA
GGGGGGCGTTTGAAGCTTCCAGGACTGGGGGCATCTCTTGTTCACAGGTGCAACCCAGATTAAGCTGAGCGGAGGCAGGAAAGGGGGGCCCTAG
GGTAGGAGAGCAGGGACCCAGCTGCCTCGCTGGCAGCACGCAGGTGGAAGTTAGGCGCGCAGGGCCCCAGACGAGTGGCCAGACCTGGCTCAGA
GCGGCTCCGGGGGGGGCGGGGAATTCCAGGAGAGGCCCCTCCCGGAGCCGCGAAACCATCCCTTCCCGCCTCCGCGCGCCTCGCCCTGCCGCCC
ACCCCCTAGCCTCCGCGGCAGCGCCGACCCGCCCAGCCAGGCTCACCGCGCTTGAGGATGGCGCTGTGGTGCGGCAGGCGGCCGCCGCCCTCAA
GCGCGGAGCAGTTCCAGCGCTGGTCGCGCAGCTGGTGCTGACACTCGTGGACCGCGATGTGCAGACCCTGAAGCGCGGACGCCGTCACGTCGGG
GTTGCGCAGGCACAGGCCTAGCTGCCGCTTGCTCAGGCCGGACAGCGTCAAGCACACGGTGTTGGCCGTCAGCGGCGGCTCGCCAGGCAACTTC
AGGCCCAGAATCTCATTGCTTAGAGCCCTGGGAACCAAGAAGGCGTCTGGGGTCTGCGGTCCAGACCCCTCCAACTCTCCCCACCCCGGGTCGG
TGTTTCTATGGCCTGGGAGACAAGGGGAACTGCTCACCGACTGCACAACGCCAGGAACAGGAGACCCGCGAGGCCCGAGGGCGGAGGCCGCGGC
CGGGGCTCCTCCAGCATGTCGAAGCCCGGAGGCTCCGGTGGGCAGATCGATCAGGACCTGCGGGACGGGAGACTTGATGCGGGTTCAGGAATAG
GGCGGCTCGCTTGGGGAACCAAGTGACGCCCTTCTTCGGGCTCCTAACCCACCGGTGCCACCCTACTGACCCTTCTCATTCCTCCTCAAACAAA
ACAAGAAGAAACACCCCTTGATTCCCAGAGTCCCTGAGGCTTGGAGGTCTTAAAGAAGAAGAGTCAAGGCGTTGTCCCAGCTCAGGACTCAAGC
GAGCACCCCTGGAACCTTGCCCAATCAGACACAACGCCCGGCACACAGACACACACTCACTTGCAAACTCAGACACACAGACTCACAAACACTT
GCTCCACAACCTCGCCTACTGCTCCCTTTTCCAGGGCCCCACGACTAGCTTCCCCGCCCTCACAGGGCTAGGGAGAGGGATAGGGAGGAGGTTC
CCCACCACCTAAGGGGGTGTCACCTCCAGGTGTCATCAAGGGCAAGTAGGCTCCATGAGAAAGGTCTAAGAATGAGTTCCCCACTCACACTGCC
CCCTCCCTGAGCAGCAAGGAAAGAGGAGAGGCTCCAGGTGCAGGAAAGATGGGGAGGAGAGCCTGGCTCCCTCCTAAAAGGGACCGTGGCACAG
ACTGAGTCCTCTTCCCTATCCCCCACCTTGCCTAACTACAAGCTCCCCTCCTCCATAGTCGCCCGTCTGGTCCTCACCCCTTGGCTCTCACATT
CATTGCATTCTAAATCTGGGGGCACTCCTTTATTCTCTGTGTCTTTATCTCTAGCAATATCTCTGGCTCTCTATGCGTCTCTGTGCTTCTGTGG
GTGGATGCGTGTCTGTCTGTCTGTCTGACTACACACGTGCCTGTCTCTTCCTCGTGTGCACGTCCCTGCCCTCCCTGCTTTCCCAGGTCTAATT
ACCTCCAGTGGTTTGGGTGCGGGGTCCGAGACAGGCACGGTTGGGTTGCCTGGGCTTCACTAGTGCTGCTTAAACCGTGGGGAGACTGCGCTGG
GTTCTGTCCCCTTGTCAGAGCCGCATTCTTCCAGGGCCGGGCCACTCCTCTTCACCGCTTCCCCAGCGCCGCCGCGGCCGCCGGGAGGAAGGGC
GGGAGGAAGGGAGGGAGTGATCGAGAGAGCTAGCAAGCCGGCGAGCAAAGGACTGGGGGCTCTGGCTTTGCTCTCACCGAGCTCAGGGTGGCTC
TGCCGGGCTGACGGAGCTGCAGCCCCTCCCCGAGCCTGGGGCTTCCCCACCCTACCCTCCACTCCCAGCCTTGACCAGGCGAATGCAGCCTCCC
CCTCCTCCCAGCGCGCGCGCGCGCGCGCGCACACACACACACACACACACACACACACACACTCTCTCTCTCTCACACACCCTCTCCCCCGACG
CCTGGGTTCAGGCTTGGCCCCACTGAAGAAGGAGGTGGCCAGAGTTCCTCTCACCTGATCTGGGGTCGCCAAGGCTGCAGGGAGGGTGTAGTAA
CCTTCCTAGGGAGCCAGAATGGGGCCCAGATGGCCACATCTTTCATCACTCCTCTTTGCGTCAGTTTCTCCACCAAGATCAGGGTGGCTGGAAG
GTGCCTTTGACAAGATGAGGATGGAGCCAGACAGTCTTTGGGCTTCTCGGTGTGGGACAGTCACATGTGGCACCCACAGCCTGAGCGCACCCGG
GGTACTCAGAGCGGGCAGGAGAAGCACACGTCTGGGCAGCTTGGTTCCCACCCTCCTCCAAGCCCTGCGCCTGCCCCTTTGGAGGGGACAGCTT
TGGCCTCAGAGTTTGGCCCTAGCAGAGGTTCAAGTAGCACCAAAGATTGTCAGCCTGCCCCGATGCTTCCTTGCCTGGCAGGGATGCTTGCCAA
CAGAGGTATCCTACCCTTCTCGGGATTCCCCCGGTCTGCACTTTGGGTGGCCTGGATTCCTGCTCCAAGCTGTAGACTTGCTTCTTATGGGATG
AGGAGGGCCTTGGGATCCAAAATTGAGCCAAGCCCAAGGGCAGCCGGGAAAGAGCCTGGTCTTGGGGTGCACAGGCAAAGGCAAACCGCCTTAG
GGAGACCCAGTGGCAGCGAACGGGTTAGGCGCCACCGTGCTCCCGCCTGACTCACCCTCGAAGCTCTAACCACTGTGGGCTATTTTTGCCAGAG
AGGGGCCCCAGGCTTTCCAGCCGCCCCCCGCCCCTGCCAGACCGCAGCCCAAGGCACGGCTGGCCCCACGCGCCCCAGGCACATTGACTCAGAC
TGCCCCCAAGCAAGCAGAGGAACAGGGATGATACCCACAACCACAGTCACCTCTTCCTTGGTGGTGGCGTTGCAGCTCTGCTGGGCAGCCAGCC
TCCAGTCTGTGCTGCTGGAGGTCCTGCCCGTGCTGCAGGGGTCACTGTTGGATAGCGGGCACAGCAGTCTGAGACAGGAGATCGAGCAAACTTA
CATCTGTTGGGATATTTACAGATCACCTTTACGTGCCAGGCTGTGAGCAGTCTGGGGAGGTGAAAGTGCCATGGTCCCTGACACTGGACAGCCT
GGACCAGACTGCTTTTCTCTGATTCCCTACCTCTGGGATGGGGAAGTGGGAGCACTAGAGTTCCTGACCCTCCTTCCCATTTAGAGGACAAGAG
CCAAACCATGTGTGGTTTTGGAGGTCACCAGAATCTGCAAGCCCCATCTCATAGTTTTCATCCTTACACCTGGATTCATGATCCCCACCAGTGT
CCTTATCCTTTTTCTTGCAGTCACCCTGGGATTCCTGCCAGGTCCTTGGAAGAGGGGAATGAGGTTGCCTAAGCCTCTCTTCTCCATGGGGACC
TCATGGACATTTGTATTTCTCCATGTGTCTCTGGGTCCCCCTTAGGTTTGGGATTATTTTTTGCCTTCTGATTCTACCCTTGTCAGGCTCCAGC
ATACAGCCAGGTGCCAAGAAGGGGCGAGGGGCAGAACTACTACAATAGGTTTCAACTTCCTGGTGACCCTAGGTCTCCCCAAGCAGGACTGTTC
CAAATCCAAGGACCCAGAGTCCTGCTCCTTCTCTCTACTGAAGCCCAACTCTGGGACAAGAGGCCACAGGCCGGCAGACTGGGTGGCCTGAGCT
GGCCCGCAGTGCCCTCAGCCCGACCGGCTGGCCTGAGCACTGACCCGCAGGCGGCTAGAGGGCGGTGGGACAGGCGGGGCCTGGAGAGGGCTGT
GGGTGCACACCCATTGCCTAATACCCCAAGCACGCGGCCAGCTCCAAGAAAGGGGAGGGACGGGAACCAGAGAGACCTTGACGTGGGTCAGGGC
CAATGACAGGAGGGGGAAAGTGGGGGGCTTTCATCACTGCAGGGAGGGACTAGGAGCAAGCTGGTTCCCCTTCCCCTCAATGAGAAGAGTCGAG
GGCTGCCCTTCCCATCCTAGGGATGGAGGGTGTAACGAGAGCAATGTGGGAAGAACACTGGGGCCCTGGAGGGCGGGGGTCAAAGAAAAGAGTG
GGGGCCCCTGGAGATTCGGGGATGTCGAGCAACTGAGGCGGTGGCTAAAGCCTCAGAAGCCTCCTCCTCCTCCTCCCCCCATGTTTGCCTTGGC
CCGGGGTCCCGGGTGACTCATCTGTTGCTGAGCCGCTGATAGGGGAGGCCAGACCCGGGGAACCCAAATTATAGGCCCAGGAGGGATGGATGCG
CCCGTGGCGGTGAGCTCAGCTGCGCTGCCCACCCTCCGCTTAATGCGCCTTCTGCTGCAGCACCGTAGGCCACCACCTGGAGGCACCAAAGGGT
CTGCGGGCCGACTGCATACTGGACTCTCAGGAAGGCCCCACTTTCAGCAGTCCACTCCAACAAATCCATGGGATTACCTTAGACAGAATTTTGC
CCCCTTCTGTACTCAGGCCTAATGGATGGGCTGTGCCTTCCCAGCCCAAGGGGGCAGTGCTGCCTGCGGGTGCTTCAAAGGAGGGTAGGCTCCT
CTGCCCACAAACTCTAAACCCTGGAGCCCTGCTTCCTCCCCAGATCCCAAAGTCAAGGCAAAGCCCCTCTCCCCTCTAACATCTCACCTCTAAC
CCTATTCCAGGGGGGTGGTTTGCTACTGATTTTCAACTTCAAGCCTTTAAAGTCATCCACGGTCAAAACTGATACAGAGAAAAATGAAGCAGGG
TAAAGGAGATTAGTAGTGGGATTCTATTTTATAAAGGGGGAGGGAAAACAAACTGAACGAACAAATACATGGAGAGATCTGAGGAAGAGCATTT
TAGAAGACAGAAAAGCAGGTGCACAGACCCTTAGAAAGGAGCATGCTTGGTTCAAAGGATTAGAAAAGAGGCCAGTGAGGCTAGTGGGGAGAAA
TTCGCAAGGAAGAGAGTGGTAGGCAATGAAGCTGGAGGGCTAGGAAGGAGGCCCCTTTACTTTGAGTGACATGGGGTCTCGCTGGAAAATTTTG
AGCAGAGAAATGAACTAATCAGACTTCTGTTTTAGGAAAGATGGCTCTGGGCTGGGCGCAGTGGCTCATGCCTGTAATCCCAGTACTTTGGGAG
GCCGACGTGGGCAGATCACGAGCTCAGGAGTTCGAGACCAGCCTGGCCAACATGGTGAAACCCCACCTCTACTAAAAATACAAAAATTAGGTGG
GAGTGTTGGTGGGCGCCTGTAATCCCAGCTACTCGGTACGCTGAGGCACCACAATCGCTTCAAACCGGAAGCTGGAGGTTACAGTCAGCCGAGA
TCATACCACTGCACTCCAGTCTGCACAACAAGAGCAAAACTCCGTCTCAAAAAAAAAAAAAAAGAAAGAAAGAAAGAAAGAAATTGGCTCTAGT
AATTAAATCAACCCTTTTGATTTTTGCAGTAAAATGGAGCACTGAAATGGAGCAATCTTGGGCAGTAATGTGGGGTCTAATGAAGTTTTTGGGT
TTTTCAGATGGGTACTATTGCAGCATGTCTGCATGCTTATGTGTATGTTCCAGGAGAGAGATAAGTGGATGATGCAGGAAAGAAAGAGGGGACA
GTTGATATCATGATTATTTGATCTAAATAGAAAGTTGGGTGCTTGTTTTGGCAGCACATATACTAAAATTGGAATGATATAGAGATTAGCATGG
CCCCTGCACAAGGATGACATGCAAATTTGTGAAGCATTTCATATTTTGAAAAAGAAATGTCAGCCACGTCATAAACAGTGTGACCCAGATCTAG
GGGCCTTACCCTCTTGCCCCCTACTCCTGGTGTGTGGAATGTTGGAACAAAGCACAGTGGCTCCTTTCCTCTCTTCCCACCTCTGCTTGACAAC
AGTCGTCAAAGACAGGGCTTCCATATTTTCCAGCCAGCCTCCCACCCTCACGGTGTTGTATCAATCCACCAGGCCAAAAGATGTGACCCAGCCC
CCAGTGGGAAGAAACTCATAAGGGATAAAGGACAGGCTCCCCGTGATACATTCTCCATTTACTTGAGCTATCTATCCTGCGTCCTCTCTGCAGG
GACTACTGGCTTTTGGATCTACGGAGGGTGCTGGACCACTACACCTTTTCCCTCTGGGGTGGATCCTTGGAAGGGCCAGATATACTAGGCTGGG
CAAAAGGGAAGAAAAAAGGGAAAGAAGGACATTTCTTTCTAAAATAACTTCCATCAGGCTTCATTTGGGTTAATATGCATCTCATTTAAAACAC
AAGTGCCCGGGAATATTAATGAAACTTACCTCGCATTTATTCCTTAGAGTGATTTCCCTGCCTTAGAAGGGAATCCTAGTCATTTCTGGCACTT
GAGACTTTAGGTTCAGGCCTGGGGAAATTTCTCAGTCAGAAGGCATCCTAAAAGACAAGGGAGATGAAAAAAATGAGAGCTAGAACTCAAAAGG
GAGGCAGAAAGGCCCAAAAAATTATTTTTACCCATCAATTTTGAGAAGGGTTCCCAGCCTGTAATTGCTGCACACTGGCAAGCAGCTGGTAAGG
TCGAAAGAGCATGGGCTTTGAGTCAAATTGGTCTGGGTTTTAATCTGGCTCTACCATTGATTCATTTATTAGACGTGGACCTTGGACAAGTGCC
TGATCTATTTTTAATTCTGCAAAATGGGGAGAGAGAAGAGATCTTCCCTCCTTCCAGGGGCCATGTGTGTGGTGGTGGGGCATGATAACCAGGC
TGGCAGTGCCCCCTATTCCCCATATAGGGAAAAGCAGCCACTTTTTTTTTTTTTTTCAGATGAAGTCTTGCTCTGTAGCCCAGACTGGAGTGCA
ATGGCGTGATCTCGGCTCACTGCAATCTCCACCTCCCGGGTTCAAGCCATTCTCCTGCCTCAGCCTCCTGAGTAGCTGGGACTACAGGTGTGCG
CCACCACACTCAGCTAATTTTTGTATTTTTATTAGAGATGGGATTCCACTATGTTGGCCAGGATGGTCTCGATCTCCTGACCTCATGATCCACC
TGCCTCGGCCTCCCAAAGTGCGGGGATTATAGGCATGAGCCACCGCGCCCAGCCTGTCACTTCTTCAATAGGAGCCCTAAATGGCCTTGAAGCT
GAGTAGGAGTCCCTGGGAGAGAAGAGAAAAGTGTACAATGGATGAGATGGTCACAGGCACTCTGGGTATCCCAGTGTGGTGGGAACTAGAGCTT
TAGGCAAAGACAGAAACTTGGCAGAAACATCCAAAGAGAAGCAAACACATGGAGGCACAAGTTTCCTCATCTAGGTTCAATGTAGCCAGCAACC
CTTGTCTTCCCAGTCCTCTCCATCACCATACATACAGTGGACATCCGCACCATTTCCCATCCTTTCTGAGCCTACGCCTCAGAGACTTAGCCAC
TCCACGCTGGGTTCACCTCAATACCATCTTGGTTGTAGGCTCGGCTCTCTCCCCCAATGACATGCACTGGTTCACACATACCACAGTGTGACAC
GCCATAGGATGCCACGAGGTACAAAGGGCAACAGAATGACGCATACACACATATTTAATCTTCCCATGCACATGCTCATCCACCCACTCCACAC
ACAGTCCAGACACTCTGCATCCCTCAATCATGCTTCTGAGTCTCCTGTCGACAGTTGCCACCTCCTTCCTGACACACTGCCCCAGGCGGTGACT
GTGACAAGGTGACTCCATGACCTTTTCTGACTTGAGCTAAATTCCAAAATTCTTTGGAAAGTTTCCTAACATCCTTCGTCAGAACAAGGAGTTT
CTGGCACGTACCAACACACAGGGAGGATGCACCCTCAGAACACAGCACATTCTCACTCCCACCCATATTCACGTTGTTCCACTTCACACACACA
CACACACACACAGCCACTTGTGCGCTTCTTCTGGCGCACATGAGCAAACTGCCTCTTCCTTTAGGTTTCTCTCCACCGCTAGGCTCCTTTTGGT
TAGCTCACCCCCACAACTCATCCCCGGGATTTCCCTGACCACAGCCGCACTCACGCCCCCGTCTCCCCTTTTTCCTTCTCTGTCCAGCCATCGG
GGGTTCCTGGGCGGTTAAGCATCTCCCCGGAGTCGCTGCCCAGAACCACAGCTTTCCTTCCGACACTCAGGATGGGGGAGAGAGGGGACGTCGG
AGGGGCCCGGGGTGACGTCGAGGGGACAACCCCACCGCGGGCGGCGAGGCGGGCTGGGCCCCTGGCGGGCTCTCCCCGCAGCACACTCTCGCCG
CGCCCCCTGGCGGATGCTAGTCCTCGACGGGCTCCGGACGCTCAGCTGAGTGAGGCGGGCGCGCGTGGGAGGGTGTCCCAACGGGAGGGGTCCG
CGGCCAGTGCAGGCCCGGAGGCGGGGGCCACCGGGCAGGGGGCGGGGGTGAGCCCCGACGGCCAACCCGTCAGCTCTCGGCTCAGACGGGCGGG
AACCACAGCCCCGCTCGCTGCCCATTGTCTGCGCCCCTAACCGGTGCGCCCTGGTGCCACAGTGCGGCCCGGAGGGGCAGCCTCCTCCCGTCAC
TTCAGCCAGCGCCGCAACTATAACAGCCGGTGCCGCCCGCCGTGGCCGCCTCAGCCCACCAGCCGGGACCGCGAGCCATGCTGTCCGCCGCCCG
CCCCCAGGGTTGTTAAAGCCAGACTGCGAACTCTCGCCACTGCCGCCACCGCCGCGTCCCGTCCCACCGTCGCGGGCAACAACCAAAGTCGCCG
CAACTGCAGCACAGAGCGGGCAAAGCCAGGCAGGCCATGGGGCTCTGGGCGCTGTTGCCTGGCTGGGTTTCTGCTACGCTGCTGCTGCCGCTGG
CCGCTCTGCCCGCAGCCCTGGCTGCCAACAGCAGTGGCCGATGGTGGTAAGTGAGCTGGTGCGGGGTCGCCACTTGTCCCGCGGCACAGAGCCA
GGGGCCAACCCTACCCAGCTCCCACGCTCTGGGATCCGTCTGCCGACAGGCTCCCTCCCCGCTCTGACTTCCCTCCGCGACACCGAAGGGCGAT
CTGGCATGAAACTGCCCCAGACTCCAGCTCTGTACAAGTGGGGCGAATGATCCGCCCGCGGAGGCCTAAGATACCCCAGGGAGCGAGCCCACTC
TCATCTAGCACCGCCCTTCCCCTTTGAGCGCCAACTCCAGCCTCACGGCGGTGGCTCACCACAGGTTTCCCCACCTCGGGAAGTGAAGGGCCAG
GAGTTCGCCTAGAAAGGAGGGGAGAAGAGGGTGGGACTCCTAAGCATTTCACGCCTTGGGTGGGCAAGAACTGCAGGCCATGATTATCTCGCTC
AGGCTGACCGGAAGAGGCTCGGAGATCCAAGGTAGACACTCGGTCTCCGGGTACCTCCTCTGTCCAGTCTCCGGACCTAGGGCTCAGGCGAGCA
GCCCTGGGACTACTGGGCACACACAAGTCTGGACGCCCAGTTCTTTCAAATTAGTGAGCCTGGGAGAGCGGGTATTATTAATCTCCCGCCATTC
TCTCCAGCCACATACCCCCAGGAAGAGGACCGGGTGGCACAGTTTTTATGGTTAGGGTGCGGATCCCCTTCCTGAGCCTGAGCTATCATACGTC
CCACCAGGGGTATTGTGAACGTAGCCTCCTCCACGAACCTGCTTACAGACTCCAAGAGTCTGCAACTGGTACTCGAGCCCAGTCTGCAGCTGTT
GAGCCGCAAACACCGGCGTCTGATACGCCAAAATCCGGGGATCCTGCACAGCGTGAGTGGGGGGCTGCAGACTGCCGTGCGCGAGTGCAAGTGG
CAGTTCCGGAATCGCCGCTGGAACTGTCCCACTGCTCCAGGGCCCCACCTCTTCGGCAAGATCGTCAACCGAGGTGGGTGCCCAGGAAGGCGAC
GCTTCCGGGAGCAGGGGAAACGCGGGGTCACCCCCAGGGCATGGGCGGGCGAGTTCAGAGAAGGTGTCCCAGGCGCCTGGAGGGTCACACAATC
AACCTTGCCAAGTGCCTCGTGCCCAGCGCCAGCTCGGGGCCAGACTTCTACCAGGCGTTTTCCAGCCGTGCACCCTGGAAACCAAGCTTAACTT
TTCTGAGCTACTGCCCCAGATAAAGAAAGTTTCGGGTCGCGGACGCCGGCTGACCGCCGCTTTCCCCCAGCCTCTCTCAAAAGCGCCTGGGAAG
CTGCTCTCTCCAGGCGTGTGTCTGGCCTCTCGCCCAGCAAGGCTTGCACCGCCAAAATGGGCCGAAAGTTTTGGGCTGCGAAGAAGTCTTGGGG
ATGTATGGTTCTTCCGCTCCCCTCTCTTCGGTTTGTCTCTCTGGGGCTGCTCCACTTCCGCTATCGAGCCAAAATGCGCCCTAGAATCTCCCAG
TAAGGTGTGATTACGCCCGTGGACGTGGCTGCGTGCCCACGCACCTGCTTTCTCTACTAGCCCTAGAGACCAGCTTTCCAGCACTGCCGGCCCT
GGCTCTCAGGACTCAAAGTGCGGAGTCGGGGGTGGGATTCCGGTCCCAAGCCCTTCATGACGGTGCTCGCCGCGCCCCGCGTACCCCCTCGCTG
ATCCCCGCTCCCTTCTCCCACAGGCTGTCGAGAAACGGCGTTGATCTTCGCTATCACCTCCGCCGGGGTCACCCATTCGGTGGCGCGCTCCTGC
TCAGAAGGTTCCATCGAATCCTGCACGTGTGACTACCGGCGGCGCGGCCCCGGGGGCCCCGACTGGCACTGGGGGGGCTGCAGCGACAACATTG
ACTTCGCCCGCCTCTTCGGCCGGGAGTTCGTGGACTCCGGGGAGAGCGGCGGCACCTGCCCTTCCTCATGGAACCTTCACAACAACGAGGCAGG
CCGTACGGTGAGCTTTGAGAGGCTCCGCACCCTAAGCGGAGCGGCAGGGGCCAACCTCGGGCTGGGGAAGTGACGGTCGGTGAGATAAGGCAAG
GGGCACCAGGAGAGGGCGTCCTCGGAGAGCCGGAGGCTTGGAACGAAGACGGAGAATAGAGGAGACAGTGGCTGAGGGCAAAGGTATGTCTCGC
CCGCGGACAGGTAGAAGAGGTTGCAAATCAAGCACAGTCTCTTCGCTGTACAGATTCGAAAAATAAGCCTGAGAGGCCGAGACTGACTCGCCGC
GGCGGAGCAGGGTTGGGCAGGGTTTCCAAATCTCAGCGGAACATTTCGCGCCTCCCTTCCCCTGGGCTCAGCTAGGCCTGGGCCTTTGCTGAGG
TCCGGCCCCCGTGGCGTCCGGGAGAGGGCAGTGTCTGGGAGGGTGACTCTGGCCCGGTGCCCTGGGACACTCTTTCTTCCCCTATCCCCGCAGA
CCGTATTCTCCGAGATGCGCCAGGAGTGCAAGTGCCACGGGATGTCCGGCTCATGCACGGTGCGCACGTGCTGGATGCGGCTGCCCACGCTGCG
CGCCGTGGGCGATGTGCTGCGCGACCGCTTCGACGGCGCCTCGCGCGTCCTGTACGGCAACCGCGGCAGCAACCGCGCTTCGCGGGCGGAGCTG
CTGCGCCTGGAGCCGGAAGACCCGGCCCACAAACCGCCCTCCCCCCACGACCTCGTCTACTTCGAGAAATCGCCCAACTTCTGCACGTACAGCG
GACGCCTGGGCACAGCAGGCACGGCAGGGCGCGCCTGTAACAGCTCGTCGCCCGCGCTGGACGGCTGCGAGCTGCTCTGCTGCGGCAGGGGCCA
CCGCACGCGCACGCAGCGCGTCACCGAGCGCTGCAACTGCACCTTCCACTGGTGCTGCCACGTCAGCTGCCGCAACTGCACGCACACGCGCGTA
CTGCACGAGTCTCTGTGAGGCGCTGCGCGGACTCGCCCCCAGGAACGCTCTCCTCGAGCCCTCCCCCAAACAGACTCGCTAGCACTCAAGACCC
GGTTATTCGCCCACCCGAGTACCTCCAGTCACACTCCCCGCGGTTCATACGCATCCCATCTCTCCCACTTCCTCCTACCTGGGGACTCCTCAAA
CCACTTGCCTGGGGCGGCATGAACCCTCTTGCCATCCTGATGGACCTGCCCCGGACCTACCTCCCTCCCTCTCCGCGGGAGACCCCTTGTTGCA
CTGCCCCCTGCTTGGCCAGGAGGTGAGAGAAGGATGGGTCCCCTCCGCCATGGGGTCGGCTCCTGATGGTGTCATTCTGCCTGCTCCATCGCGC
CAGCGACCTCTCTGCCTCTCTTCTTCCCCTTTGTCCTGCGTTTTCTCCGGGTCCTCCTAAGTCCCTTCCTATTCTCCTGCCATGGGTGCAGACC
CTGAACCCACACCTGGGCATCAGGGCCTTTCTCCTCCCCACCTGTAGCTGAAGCAGGAGGTTACAGGGCAAAAGGGCAGCTGTGATGATGTGGA
AATGAGGTTGGGGGAACCAGCAGAAATGCCCCCATTCTCCCAGTCTCTGTCGTGGAGCCATTGAACAGCTGTGAGCCATGCCTCCCTGGGCCAC
CTCCTACCCCTTCCTGTCCTGCCTCCTCATCAGTGTGTAAATAATTTGCACTGAAACGTGGATACAGAGCCACGAGTTTGGATGTTGTAAATAA
AACTATTTATTGTCCTGGGTCCCAGCCTGGTTTGCAAAGACCACCTCCAACCCAACCCAATCCCTCTCCACTCTTCTCTCCTTTCTCCCTGCAG
CCTTTTCTGGTCCCTCTTCTCTCCTCAGTTTCTCAAAGATGCGTTTGCCTCCTGGAATCAGTATTTCCTTCCACTGTAGCTATTAGCGGCTCCT
CGCCCCCACCAGTGTAGCATCTTCCTCTGCAGAATAAAATCTCTATTTTTATCGATGACTTGGTGGCTTTTCCTTGAATCCAGAACACAACCTT
GTTTGTGGTGTCCCCTATCCTCCCCTTTTACCACTCCCAGGCTTGGAAGCTTACCTCTCTCAAACAAATAGAATATGCTGTTGGGGGACTGGTT
TCCTAACCTTGCCAGAGGACCCTGAGATTCTGACCCTTGGATGACCCTAACGAGGCCACTGGGAATCCAGCTCTCCTGAGAGAGGAACCTGGTC
AGTAGCCAAACAACCCTGCAGGGTTCTTCTTCCTCGTGGAACAGCAACTGGCTGTGTGATATGGACAGGTGCATATACTTGGTGAATGGGGGTT
AAACAACCTCAGTGCCTGCTTTTCCTGCCTGGAACAGCTGTTCAGAAAATAGCTACTATTGAATGATAACTGTGACCATCTTCCAAAAAGTAGG
AAGAGAAACTAAAGCAGAGTGCTGCTGCCCCTAATGTGGAGCTATTGGTCCCTGAAGCTTGGCTTCTTCAGCTGTGGTCAGGGCATTGGACAGG
GGTGAAGCCCTTCCACCAACAGAAAAGTAAATACTGGCCTGGGGCAGTGGCTCATGCCTGTAATCCCAACACTTTGGGAGACCGAGGTGGGTAA
ATCGATTCAGCCCAGGAGTTTGAGACCAGCCTGGGCCACACAGGCAGCCCCACCTCTACAAAAGAAAAAAAAAAAAAAAAATAATATATATATA
TATATATATATATATGTGTGTGTGTGTGTGTGTGTGTGTGTGTGTGTGTGTGTATATACACACACTATTAGGTATTATATATGTATATCTATCT
ATCTATCTAATAAATACCTTATCAGGAGCCCCACTCCCAATCTTGGGCTCAGGGAGATCCAGATAATAGGTGATGAGCCTGGTGCTGGACCGAC
AAGCTCAGAGATGCTGGTGGGAGAGAAGGGAGCAGGTAGGCTAGAATCCAAATGCAGGCTAGGCTTAAACCGATGTAACCAAAGGAAACCCTAA
TGTGGTCCATAACCAAACTACTAAGCTAGCCATTCCCATAAAGAACCAGAATGAGGATTTTGTTACTAACACCACCAGTGTGATACCTCTGTGT
GCACATTCACCAGAGTCAGTGGGAGTTCTAATCCTACCCACGGCCCTAAACCTATCTCTAACTCTAACCCTCTTCCTGTTCCTAGGTCTAGGTT
GAACCTTAGCCTTAACCCTAACTTCAGACAAACACTAACCCCTTCTAAGCTCTAATGTCTGTCCCTAATCCTAACCCCTCCCCACAGGGGAGGA
TCAGAAGGGCAAGGGGAGCTGTGAAACACCCAACCTATCCCCTAAGAGCAACAACTGCTCCCAGCCAGCTTGAGAATTTCCCCTTCTCCCCTAT
TCCCTCCTCTCCTGTTCAGGCCTCTTCTGCTGTTGGTGCCTCCCACCACCTTTGGGATATGTGTAGGGGTAGGGGAATGGAGCCAGGAACTGCT
CAGTCCTGCAACGACATCCCTTTCCCTTCCTTTCCACCTGCCAAGCTAGCACCATCTCAGTTCTGATTCCTCACCCTAGGGGAAGAAATAAAAA
CCTATTGATCCTGGGGAAGAGGCACCTGAAAAGCTTTGGGTTTCCATAAAGAGCACCCTGCAGAGAATGCCCAGTCTATAAGCCAAAGGGCTAT
TAGATCCCTCAACTCCCCAAATAAGAAGTTAATAGGCTGCCAGAGGTGGGAGGGGGGGCTTGGGCCCCTGCTGGACAGTGCCCAGAGTTGAGAT
GAAGCAGCAGGGTAGAGTCAAAGCATTGGCAGCTGTCTCCAACTACTTTTGCCTAAATTCAAACTCTAAGAATTGGAGTCAGTATCAGCTTTCA
GGCTTGGAAGCTGGGGCACTCTGAACCAGGTTGACAAACAGGAGCTGGAGGAAGAGCAAGGGAAAGTGGTGAATTCTTTTCTGCCCCTACCCAG
ACTTTCAGCAAATCTCGTACTTTCTCAGACTCAGTTGCCTTGCCTATCAAACAGGGATGGTTATTGTTACTTAACCTTTCAGGGAGGTGATGAG
GTTCAGATGAGATGATGTACATGAAAGCCTGGGTCATTTGAATACTCACTGACACCTCTTCTATACCAGGCATCTGCCAAATGTTGGCAATACA
ATGATTTATAAACCATAGTGCCTGCTTGGAGGAGGTTGACAGTCTGGCCAGGGGATCAAGCACATCCTCAGATATTGTTAGCATATGGAGGGAG
TGCAATGAAGGCTCAATGAAGTGTAAAGCCTTCTCCACATAAATGAAGTTTAAGGGCAGACTCCCATACTCAAGCCCTCGGCATGGAGGCTCTT
CTCTTCCAAGGGCTGGAATTTCAAACGTGATGGTGCTGGGAAGTGCAGAATTAGGCTGGGCCCTCAATGGGCACTAATAAAACACAAACACACA
TACACACACACACAAACAAGTAAAAGGGCAGAGGGATCTGCAAAGCACTACATAAAAGCTAATCATTTTGTTATTGTGATCATCTATGCTATCC
TCACACAGGGCTGGAGCGGGTACATCCTGAGGAGAAGAACAATTATGCATAGAAAGAAGACTCACAAAGGTCTCAGGAGCCACAGTAGGCTCAG
GGTGGTGGCCATAGGATGGTGTCATCAGGACTGTGCTTGATGCGCCACCTCTCACCACCTCTGATCTTCAGGTGCCAGGGCGCTGGATACACGT
TGGCCTGAGCAAGTGAGGGAAACACTAGTCCTGAAATATCCTGGGGGGAGTGGAAAAGCTGAGAAGAAAGGCAGGCTGTGCACAGTCCCCAGGG
GGTCAGCAAGCCAGCACCACACACACTCTGCCACACGCACCCTGGAAAAACCCTGTCAGTGGGGCTGCAAATACCCTAGGGTCCAGCCTCCCCC
AAAGCCTCCCCCCTCGCCCCAGGCTGGCTCAGTCCTCCTCTCCAGCGCAGAGGGAGAAAAATAACAAAACGAGGAGAAAATAAGCTGTTTGGCT
GTGGTGTGAGCAGGAATATCCATGCAGGGCTGCCCACCACTCACCCTTACTCACTCTTTCCACACCCAGCCGGGGCGCCCAGCATGAGACAGAC
CACCCCCCTGCCCCCTCCCCCGTCTGGTCTAAAAGCAGACGCTGGTGAGCGAGCAGCAGGTTATAAATATGCCCAGGGAACTGCCGCCAACTCG
CTCCCTTGTCATCCCCTGGGAGGGGCAGGGTGGGGTGGCTCCCAGGGAGGAGCCAGGGCTGCCCTGTTCCCTGAGGCCCAGGAGGAAGTGGGGC
TTCCTAGAGGCTACCATATTTTCCCCCCTTTTCAAGGTGAAGGAACCACTGCCACTCCATTTTCCCTGAACATTTGCTCATCTCTTCAGATATG
TGGTCCCTACCCTCCCTGGGCCCTGGCTGGAGGCTCGGAATGAAAGGGCCCAAAGTACTCTGTGTCATGGCACAGCCCGCTGCTACTTCTCTTC
TTCACATGCATTTGCTCTCAGCAAAACAGAAGTTCATTCATTCATTCAACAAGCATTAACTGAAAGCCAGCTGAATGCCAGACCCTGTGCACAG
GCCCACCTGTGTGCACAGTGTCTGCACACACAGGTGCAAGCACACACAGCTGCCCCTACTCCATGTGCTCACATCACCTCCAGCCCCCACTAAG
GACTTCCTGGCTTCAACCTGCCAGCCTCATCAGGAAAGCAAAGCTTCAAGCTCCTGGTTGTCCCTCAGAGTAGGTTAGGGGGCATGGGGTAGGG
GGTGGCTGAGAACAGGACCCCAAACACTTCTGCTTCCACTACTTCCCCTGTAAATGAGGGGGTCCTTTCTCTCCTTCCTGCCCAATCATCCTCC
CTAAGTAGGTGCCATGTGACGAAGAAAGAGCCATTCTTGGGATGCTGAGGGTACCTGTCCAGGCCACCAGAGCCTCCCACCTCCCAGCAACTTG
CCAACTAGGTGCCAAGCTGGGGCAAGAGGCGGCACTTCCACCCTGTGAAGCTCCCAGCTCTCCTCCCCCAGGGCTGAACCCCTCCCTTTTCCCT
CCTCCCTATTAATCCTCGGAAGTTAAGGGGCCGTCCCTGGCGGCCCCTCAGCCCTATCGTGGCCCCTCACCCTCCTCGGCTGGCGTCTAATTAA
CTCCTCCTGCCCCTGGTTTTGTATGCCCCCCTTACCCCACCTTGTCGGCCCCGCCCCTCTGCTCGGCCAGCTCCCCCCCACGGACATTCCAGCC
AGGCCGGGCCAGTGTGCCAGGGCAACCAGGGCCGGACCGAGGCCTAATCCTCCCGCCGCCTGGCCCGGCTGGGGGAGCCCCACTCACCTCCCCA
CTCTCTGGGGGCTGCCCGGCTCTGCTGTGGCGGAGACACACAATCGGGACAAAGGCACAGCCCCCAGGGGGCTAACTCAGCCTTCACGCCCAGG
TTGTGCGGCATTTGGGGAGTTTAATGAATTCTCCATCACGCCTTTCAGGGCCCGCCCGTCAGCCTGGATTAATCTTCGGAGCCCTGGTGGGGTA
GGAGACACTAAGCCTGTATTTATTACTCTCCCATTGTCACTAATTGAGGTAATTATCTGTGACTCTGGCCTGGCCAACAATGAGGCATTCACTC
CTGGGACCTCGATGGGCCTGGCTCCCACTCTCAGCACCAGCCCTCCCTCCCCTCCTCTTCCAAACCCGCCAAGGACTACGATGGGCCCTGGAGC
ATGTTGGGAAAGTGGACAGCGTCCCACAATGCCTCACCTCCCCACCGGCGTCTCCCATAGCCATCACTACAGAGCTGTCTCCTGGTCCCCCCAG
AGAAGGTGAGAAAAGAAGGCAGCCTCTGCCAGGGGATGGATTTGCCTGGCAGAGCCCTCAGGCCAGAGAGCGACCTGTTTCTGAGGACTTTGGT
TTTCTGGACTCTCCAACTCCTCATCTCCAGATGCATGACACCAGCGTCTCACAAGGTGCTTTTGCAGATCCATCCTCATGTCCCTATAGCCTCT
AGGGTGTTAAGAGGCTCTAGCCTGTACCAGCCCTACTCTGGGGAGGTCCCTAAACCTACCAGTCCTGGCAGAGTGGCTGTGGATGTGATGGGAG
ATGTTAAGAGAGACAGCAGGAAAGGTTCTGTCAGGGGAAGAGCAAAAGGAGCCAAGTCAAGAAGGAGCAGTCAAGAGTCACAGCATTCCCGGGG
GTCACACTAATGTGGCCATGCAGCAACTTCAGCGAGAAACTTGGATTTGGAGGGGCAAGAGTTCCAAACAGCGGATTTCACGTGGACACAGCTT
TCTGGGTCAAGGTGGTTGGCCCTGGGGAGCAGGTATGTGTGTACAAGAAAGACAGAGAGAGAGAGAGAGAGAGAGAGAGATAACAAAGGAAGAT
TTAGGCAGAACTAGATTGTGAACTCCTTGAGGGCAACAACTATGTCTTTGTGTCTTGGTCACTGTTGCTTTTATGAGAGCTTAACATTCTACCT
TGCACGTAATAGGCACCGTCAAGTGAATGGATGAATACAGATGAAGCCGGCACTTGCTTTAAAACTGGGATCTGTGAGCTATTCTCTTCCACAG
CTGCACTCAGGCTCCTCATTCTCTCAGGACTTGCTGGAAGGAGATGTCTAGACCATCCCTGCACGGTCAGCGGTGGGCACTTCACCAGCTGTTG
ACACGACAGACCCTGGCCGAACTCAGAGCGGTAGTGGGGATAGCGCCACCTTGTGGTCACTCTTGGGAGAGCTCCTGCAGAGACGCTAGCCAAG
AAGGCAGGGTCATCTCTCAGGAGCAGGGTATCACCCCGCGCTGCAGCCATCCTTCTTCCTCTATGTCTGAACTGCATTGTTATGCCAGCGGTCC
TGTGGCACAGTTTTATCATTTGTTCCTTCTTTATTCCAGCATTTTCTGAGAGCCTGCTCCACGCCAAACTTGTCGGGTATACAGAGCTAAATCA
GCTAAATCACCTCCAGCAGCTCATGGTCCAGCAGGGGATTCCACAAGGTAACAAGACCAACGAGGTTACGATCAAGCCGCAGGAAAAAAAATTA
TCCAGGTGTTGAGGAGGCACTAGAACATTCCCAGCTCAAAAACTCCAGCATTCACCTCTCCCTCAATTCCTCTCACCGAAGTGCAAATGGTTTA
TCTTCATTACCAAGAAACAGTCTGGGCATGGTGGCTCACAGCTGTAATCTCAGCACTTTGGGAGGCCGAGGCGGGCGGATCACTTGAGATCAGG
AGTTCGAGACCAGCCTGGCCAACATGGTGAAACCCGTCTCTACTAAAAATACAAATAAAAAATTAGCCAAGAGTTATGGCGCCCACCTGTAATC
CCAGCTACTCTGGAGGCTGAGGCAGGAGAATCGCTTGAACCCAGGAACTGGAGCAGAGATCACACCACTGCACTCTAGTCTGGGTGACAGAGTG
AGACTCCATATCAAAAAAAAAAAAAAAAAAAAACCCAAAAAGCAAAAAAGACAAGAAATACTTCAAGTTCTGGCTCCCACACATCTTTCCAGGA
CTCACTCTTCTGCCCCACTGCATTCCCTAATGCCTCTGCTCCCTGTGAAAGCCACGTGTTTTCCTACCTCTCTGCATTTTCCCACCTCTCTGCA
TTTTCCCACATCCTAGAATGTCCCCCTTCCCTGTATCCCACTGTTGGAACCCATCTCAGATGTTCCTTCCTCCATGAAGACTCTTCTGATTTCC
CTTGGTAGAGTGGTCTTGCCTGTTCTAGCTCCTGAAGCACCATGTAGGTAGGTGCTTTCATTAGCAGCACATACTGGGTGTCCCATTGGTGGTT
GTCTGTGTATATTACTCATCCTCCTACTAGGCTAGCCCTTGCAGCACTGACCTGGGCATATCATATGCCTTTGTTAAATGGTGAATAGACGGAT
ACATGAATAAATGGATGCACGGACAGAGGAATAAATCGATGGAAGTATAAATGGGGCCAGGCGCGGTGGCTCATGCCTGTAATCCCAGCACTTT
GGGAGGCCAAGGCAGGCGGATCTCCTGAGGTCAGGAGTTTGAGACCAGCCTGGCCAACATGGTGAAACCCTGTCTCTACTAAAAATACAAAAAT
TAGCCAGGCATGTTGGTGGGCGCCTGTAATCCCAGCTACTCAAGAGGCTGAGGCATGAGAATCGCTAGAACCCTGGAGGCGGAGGCTGCAGTGA
GCAGAGATCTCACTAATGCACTCCAGCCTGAGTGACAGAGTGAGACTCTGTCTTAAAGAAGAAAAAAAGTATAAATGGCCAGATAGATGACAAA
TAGGTTGGTGGATGGGTGGATAAGTGGATGTGGAATGTATAGATGGATGGGTGGGTGGATGGATGAATAGATGGATGAATGGAAAATCTATCCA
TAAAAGGACTCACAAAAGGCTATGTAGGCTGAGTGCAGTGGCTTATACCCGTAATCCCAACATTTGGGGAGGCCACCATGGGAGAATCGTTTGA
GCCCAGGAGTTTGAGATCAGCCTGGGCAACATAGGGAGACCCCGTCTCCACCAAAAAAAAAAAAAAAAAAAAATTAGCCCGGCATGGTGACACG
TGCCTGTGGTTCCTACTATTCAGGAGGCTGAGGTGGAAGGATCACTTTTGCTGGGCAGGCTGAGGCTGCAGTAAGTTGTAATCGTGCCACTGCA
CTCCAGCCTGGGTGACATAGCAAGACCCTGTCTCAGAAAAAGAAAAAAGAAAAAAAATGGCCAGATGTGGTGGCCCATGCCTGTAATCCCAGCA
CTTTGGAAGGGTGAGGCAGGCAGATAGCTTGAGGTCAGGAGTTCGAGACCACCCTGGCCAACATGGTGAAACCCTGTCTCTACTAAAAATACAA
AAATTAGCTGGGCGTGGTGGTGCGCGGCTGTAATCCCATCCACTTGGGAGGCTGAGGCAGAAGAATTGTTTGAACCCGGGACCTGGAGGTTGCA
GTGAGCTGAGATCGCACCACTGCACTCCAGCCTGGGTGGCAGGAAAGAAAAGAAAATTTTAAAAAAGCCCAGGTAGCACCAGAGGCTTCAAGGG
AGGGAGCACACCCCATGCTAGGATATGCAGAGGCAGAGGCAGTATCCAGGCCAAACTTTCCTTCACTAACATCTGCACCCTCAGCAACTTGTCT
CCCAGAGGCCTAGATACATTCCACTCCCCATAAGAAGTCATGGTCTGGGAGCGGCTCCTCTTTTCATCCTCCCTGCTCCCTCAGGAATGCTTTC
ATTAACATATATTATGCCATCTTCCTGCACTGCAATGACTGTGGGTTTCCTCTTCCACATCTCTTTCCCTATATCATACACACCCTAGCTTGAG
AGCTCCCTGAAGGGAGGAAGCATGCCTGAGTCTCTTTTGTGTCCCTAGCACCTGGCATATATGTTTAATGAGTGAGTAAACAAATGAACAACTG
AGTGAGTGCTGCCTCTTCCCCATAGACACATTTTATACACTTCCTGCGTCCTCTTGTTCACTTATCTCTCCTGGCCCTCTCCTCCGTACACACA
TCACACTCACACTCCTGACCTTGACACTCTGAAATTCCAGAGTTATATGCTGGGGCTCGGTTTCCTTACCTATTTGTGGCTGATCTAGGGAAGT
CCCAGCCCTCCCCTGAGAGTCTGAAAGAGACGGCTCTTGACTCCTCTAAAGCCATATTAACTGGGTCCCATGGAGGGCAGAAAGGGGCTTCCCT
AGGAAGAAGCTCTCTATTGGGAGGGTAGCAGGATCCTGCACTAACCTGAGGTCTGCTCCCCCAAATTGACCACATCCTCCTGTGTCTGCTGTCC
CTACTCCTGACCAGTGAGGCTGTCCCAAGGTAGGGGGTGGTTGTGGCCTCTGTCTCAAGGCTGGCTGGCAGACACAGCCAGATTTAGAAGTCAT
TTTCGCAGCAGGGAGCCTCCAGTGTGCCACCCACAGAGCCCTGGAAGGTCAGTCTGAGATATGGCTCACCAGGGGCTGGCCCCTAGACCTGGAG
TCACTCCTCCCCTTCTTGTCTCTCTCCTTTTTTTGCCCAGAATTTTGGCAGCTGGTGCTGACTCTATCCTTAGCCAAAGATGGAGTAAGTTCTG
GGGGAAGGGGTGTGCTGCACTGACACTCTAACCGCCCAAGCACTGCCAGGGCAC
HUMAN SEQUENCE - mRNA
ATGGGGCTCTGGGCGCTGTTGCCTGGCTGGGTTTCTGCTACGCTGCTGCTGGCGCTGGCCGCTCTGCCCGCAGCCCTGGCTGCCAACAGCAGTG
GCCGATGGTGGGGTATTGTGAACGTAGCCTCCTCCACGAACCTGCTTACAGACTCCAAGAGTCTGCAACTGGTACTCGAGCCCAGTCTGCAGCT
GTTGAGCCGCAAACAGCGGCGCCTGATACGCCAAAATCCGGGGATCCTGCACAGCGTGAGTGGGGGGCTGCAGAGTGCCGTGCGCGAGTGCAAG
TGGCAGTTCCGGAATCGCCGCTGGAACTGTCCCACTGCTCCAGGGCCCCACCTCTTCGGCAAGATCGTCAACCGAGGCTGTCGAGAAACGCCGT
TTATCTTCGCTATCACCTCCGCCGGGGTCACCCATTCGGTGGCGCGCTCCTGCTCAGAAGGTTCCATCGAATCCTGCACGTGTGACTACCGGCG
GCGCGGCCCCGGGGGCCCCGACTGGCACTGGGGGGGCTGCAGCGACAACATTGACTTCGGCCGCCTCTTCGGCCGGGAGTTCGTGGACTCCGGG
GAGAAGGGGCGGGACCTGCGCTTCCTCATGAACCTTCACAACAACGAGGCAGGCCGTACGACCGTATTCTCCGAGATGCGCCAGGAGTGCAAGT
GCCACGGGATGTCCGGCTCATGCACGGTGCGCACGTGCTGGATGCGGCTGCCCACGCTGCGCGCCGTGGGCGATGTGCTGCGCGACCGCTTCGA
CGGCGCCTCGCGCGTCCTGTACGGCAACCGCGGCAGCAACCGCGCTTCGCGAGCGGAGCTGCTGCGCCTGGAGCCGGAAGACCCGGCCCACAAA
CCGCCCTCCCCCCACGACCTCGTCTACTTCGAGAAATCGCCCAACTTCTGCACGTACAGCGGACGCCTGGGCACAGCAGGCACGGCAGGGCGCG
CCTGTAACAGCTCGTCGCCCGCGCTGGACGGCTGCGAGCTGCTCTGCTGCGGCAGGGGCCACCGCACGCGCACGCAGCGCGTCACCGAGCGCTG
CAACTGCACCTTCCACTGGTGCTGCCACGTCAGCTGCCGCAACTGCACGCACACGCGCGTACTGCACCAGTGTCTGTGA
HUMAN SEQUENCE - CODING
ATGGGGCTCTGGGCGCTGTTGCCTGGCTGGGTTTCTGCTACGCTGCTGCTGGCGCTGGCCGCTCTGCCCGCAGCCCTGGCTGCCAACAGCAGTG
GCCGATGGTGGGGTATTGTGAACGTAGCCTCCTCCACGAACCTGCTTACAGACTCCAAGAGTCTGCAACTGGTACTCGAGCCCAGTCTGCAGCT
GTTGAGCCGCAAACAGCGGCGCCTGATACGCCAAAATCCGGGGATCCTGCACAGCGTGAGTGGGGGGCTGCAGAGTGCCGTGCGCGAGTGCAAG
TGGCAGTTCCGGAATCGCCGCTGGAACTGTCCCACTGCTCCAGGGCCCCACCTCTTCGGCAAGATCGTCAACCGAGGCTGTCGAGAAACGGCGT
TTATCTTCGCTATCACCTCCGCCGGGGTCACCCATTCGGTGGCGCGCTCCTGCTCAGAAGGTTCCATCGAATCCTGCACGTGTGACTACCGGCG
GCGCGGCCCCGGGGGCCCCGACTGGCACTGGGGGGGCTGCAGCGACAACATTGACTTCGGCCGCCTCTTCGGCCGGGAGTTCGTGGACTCCGGG
GAGAAGGGGCGGGACCTGCGCTTCCTCATGAACCTTCACAACAACGAGGCAGGCCGTACGACCGTATTCTCCGAGATGCGCCAGGAGTGCAAGT
GCCACGGGATGTCCGGCTCATGCACGGTGCGCACGTGCTGGATGCGGCTGCCCACGCTGCGCGCCGTGGGCGATGTGCTGCGCGACCGCTTCGA
CGGCGCCTCGCGCCTCCTGTACGGCAACCGCGGCAGCAACCGCGCTTCGCGAGCGGAGCTGCTGCGCCTGGAGCCGGAAGACCCGGCCCACAAA
CCGCCCTCCCCCCACGACCTCGTCTACTTCGAGAAATCGCCCAACTTCGGCACGTACAGCGGACGCCTGGGCACAGCAGGCACGGCAGGGCGCG
CCTGTAACAGCTCGTCGCCCGCGCTGGACGGCTGCGAGCTGCTCTGCTGCGGCAGGGGCCACCGCACGCGCACGCAGCGCGTCACCGAGCGCTG
CAACTGCACCTTCCACTGGTGCTGCCACGTCAGCTGCCGCAACTGCACGCACACGCGCGTACTGCACGAGTGTCTGTGA

TABLE 4
MOUSE NOMENCLATURE
ICSGNM Rasgrp1
Celera mCG14557
HUMAN NOMENCLATURE
HGNC RASGRP1
Celera hCG38304
MOUSE SEQUENCE - GENOMIC
CACTTTTCAAATAACATGAAAGTGAAAACCTGTTGGCAAGACCAAAAGAAGTCTGGGGTAGTGGTTCTTCTTAGGTCATTTTAGAGATTCTTAG
ATCTTGAGACAGAGCAAAGAACTGCTTGTAAATGAGCCAAGAAACTAATCTCCATAATGCTCAGGGTAGAAACTTGTAGCTTACAAGACACTTC
AGAGGTTAGCACCGACAACCATCTATTTAGAATGCGTTACGCGTCCCTTTTTCTCACACAGTACTTTTTAATGACTGATCGCTGAGAAGCTCAA
ATGTTTTTGGATACAGAATCTAAGCTTGTCACAAAGGCGTAGTTAGCTTTTCTTCCACATTTGCTTCTCACAAGCTGTGTTCGGACAAGTGACT
CGACCCCTCTAAGGCTTAAGTTCCTCATCTACAAAGCGAGGGACCATGACAACTGAAAACCAACACAGTGTGTTGTGAGGGTGAATGAGGTCAC
AGGCATGGCTTCCTCAGAACAACTTCTGGTCTGCACAAGTCATTTCCTCAGAAGGAGGCTGGAGAATCGGAAAACAAGGAGGTACAAATAAATA
TTTTAAACAAAGTACTAATGAGTGAACATTCCTTTTTAAATATTCTGCTCCATCCCAGCACCCAGATCAATGCAAATTAGCATGAAGCCAGACT
TTGCGGTTTATTTGTTTGGTTGGTTTTGCCTCCTACTAATCTTGCCTGATGGTGTAACTGACTTTTCTCAGGGATCCAGAGTTGCTGTGTCCTT
TCTGCTTGGACCTCTCAGGATAACAGCTCCAAGTGTGACCTGGGACATAGGGGACATGTTATGAACCASGACAAGCCCTTGGGAGTTGCTATGC
CAGCAGCTAGTTATGGGGTATGTATGGCTCTTTGGGCCTCTTTAAATGAGAGTCTAGTTTTATCACATCTTGGGCCTCTAATTCGCTTGGATTG
AGATGGGAGTTCTTTGTTTACACGTTGAGGTCAGGACTGTACTATGTACCCCAAGCTGGCATCAACCTCACTATATCCCTGCCTCAGCCTTTTT
CAAGTTACGGTTTTTATATTTTATTTTTAACAGCAGTTTTAGGTTAGAAGCAGAATTGTGTTGAGCATCATCCTGTCATCTCTACATGTGCACA
GTACCCCTCTCCAGAGTGACACATGTGTGACATCTGATGAACCTAGATCATTATCACCCAGAGGCCATGGTTGACATTGGAGTTCTATAAATTT
GAATGACCATTCACTGTCTAGTGTCCCCACACACATTACAGTGCCTTACAAAGTAGATTCACTGCCCTGAAAAGTCTGCGCTACAGCTTTACTT
TAAAAAAACCAAATACCAGGCCTATTTTCCAAATTTGTTATCATAGCATTTGGGAGATGATGGCAGAAAGGAGACAAGTTTATGATCTATATTG
AATCCGAGGCCAGCCTTGGCTCATGAGACCCTGTCTTAAGAAAATAAAAGCATAAAAATAAAAAGAATGTGTATTTAAGCCCCCTTTCCACCTC
ATATACCACTAACTGTTCTCCTCCTTCTCCTTCCTGTGAGTCCAAGGGACCAGGAAATGGCTCATGTCTAAATCCACCCATGACAACTAACTCT
TCCAAGAAGGTGCAAGTCTAACTGCCAGGTGCCGTTGCCAGTGGGTCCCAGGCTCGGGACTCATAGCAATCACAATATCCACAAAGCAGGTTTC
AATTTCACTACTTTTATAATCAGACAAAACACATATTTAGGGTTGGATAGATGACTTGGTGAATAAACCACTTGCTGTCTGAGCATAAGGACCT
GAGTTCAAGTCCCCAGAACCCTTAGGAAGATGGTAGCGTTTATCTTCAGTCCCAACTCCTTCTGGCAGSATGAGAGACAGAGACAGAGACAGGT
GAGTCCCTGGAAGATCAGGAGCCATCTAGCTCAATTTACACAATAGAGAGCAGCAATGAGAACTGGTCTCCAGTAAGCTGGAGAGGAAGGTGTG
ACAGCCAAGGTTTTCCTCTGACCTCAGATACATGTAGGACACCTGTACTCACACACAAAAGCATGCACATTACATGCATCCACACGTCATGTAC
ACACACACACACACACATGCACATGCACATGCACATAGACACATAGGCATATAGGCATATTTAATAATGAATAAATAATAATAAGAATCCTCTA
GATTATATTCACCTTTTTGCCACTGCAGTTACAAAATGTTTTTTTTTTTAATTTTTATGAAAGAGAAACAATTCTGCTGCATTTAAGAGCAGGG
TCTTAAGACACGCAAATCGGTGACGTTATCCTCTACTTTGCCTCTACTCTTCCGATTTCTTCCTCTTCCATTACCAGGCAAAACTCACAGCAAC
ACTGGGTTCTATGGGCTTGTCTAAAAAAAAAAAAAAAAAAAAAAAAAAAAAAAAAAAAAAACTCCAGCAACAATCACACCGAACTTGTTTATTT
CATGTGCCTATCATAAGCCTTCCCCAGATTTTTCACACCCTCCTCCCAGATACAAACTAACAATGTCACAGCAACTAACAGCACAGTGGGGTCA
CACCTGCATGGGCCAAATTAAGATCCGCCATGAGGTGACTGTCCTCTGGGAGCGGCTCTCGCTTCAGAGGCTCTTGTGAATCACACACAGCAGC
ACCATCACACTTCCCACAGCAGGTGGCCATGTTTTTCACTGACTAACGAGCAGGATCAATGACTGAACACTTCACTAACACCTGGTCGAGGGCT
GATCTCCATCCACCCTGGGAAGCTGAAATGAAGTCTTCTCTAGCATTTCCTATCTAATTCAATACAGAGGAGGGGAAAAAACATTAGAGAAACA
AGATAAACAACAACAAAATGACAGTATTTCTATTACATTGAATTTTGTGTGTGGGTTGATACTGAGAGATTATTTCCACCAAAATGTTTGAGCC
AGTTTGCCAGTTAGCAAACTAGGCAGTCATGCAAACTAATCTAGATCCATCCTTGCAACAAACTGTAGCTCCCATCCGGCATTGTGTCCCCCTA
AACAATGCAGTTGTGATTGGCACAATCCAGCTCACATTGTCTCTATTCTGTCTAAAACATGCATTGCATGTGGAAAATACAGTAGCCTACTCTC
TGCTAACTGCCTGCCTTAGTCTCACCTAGAGTTTTCTCTCTACTTCTCAAGCCAGAGGGCACAGAAGACGCAGACATAGAAAATGATCACTCAA
ACCCTATGTGGATTTTCATGGGAATATTGTCTGCCCTTAAGAATGCCACCCTCTGCTCTCACGACAGTGAATCAAACTGAATTGAATGACAAAA
CCTTAAGCCTGTAAAGGATAGGTACGCTTGGTGCAGGAATGGTAGAAATGGCTATTAGAATGGGAAGGTCAAAATCAAGTCATTTGTGGTTGTG
TCTGAAGTTGATTCCTTTAGGTTTCATTAAATAAAAAGAGTGAGGGTTATGGAGACATGATCCCCACCACTGAAGTAAAAGCTGGGTGAAGGCA
CATAGGTACCTGCGTCCCCCGTGCTTTAGGGTGGACATTGGAGAGTCCAGGACGTCTCTGGCCAGCCAGCCAGGCCAAAAGAGAAACATCAGGG
TTAGTGAGAGACCCTGGATTGAGGAAACAAGACAGCTGTCATCCTCTTCTGATCACAACCCCATGTGAGCACATGGGAACACACACATGTGTAT
AGTACAAACACAGCAAACCTACACAGAGACAGACAGACAGACACAGAGACAGAGACAGAGAGAGACTTCCCTTTTAAAAGGAAACGTTTCCTTC
CCAAGCTTAACGATACATCTGCCCAGTCTTTGAGAAATCTCTCCCCACATTGATTTGCCATTATTTTCTCTGCTACTCCCATGCAAGTTGGACT
ACAGATTTTCAGGAGTTTTTGTTTCAATGCTGTGTTTATCACAGCACTTTTTTGAATGCATATTGAATTGAGCAGTTTAAGGGTTTGGTTTTGG
TTTGGTTTGGTTGGCTTTGTTTTGTTTCTTTGTTTTTACCCAAAAAAAATCACGTCTGTTTTTTTTTTTTTTTTTTTTGGTTTGTCCAGATGTT
TGGATCTTTCTATAAACTGCACCCCACCCCAGCATTTACCCATGCACCTTTGCAGGTGATAAGTGATTAAGCAGAATGAGTTTACTGAGGTATA
CAATACGAACTTTATAAATAGTGTTTGTGTGGCCAAACTGGTTTTAACTTTGGAATTGGTGCTGGTAGACTGTACTGTCGGCCTCTGGAAAGAG
GCCCAGTATTGAATTTTAATCTCTTCAGCCCTCTTTTTAGACCTACTTGTCCCAGGCACCCACTGGCAAACCTGAGGGTGTGTGCTAATTGGAT
CACACCCAAAGGGGAGAGCAGGTAGAAGGAGCTGGAGCAGACTTCGGAGAACCAGAACCTCCCCGCCCTGCCAAGACAATTGAGAGGCAAACAG
ACTGATACACACCAGTGGGTGAGGGGAGGGTGTGTCGCCCCAGTATTGTGGTTCTGCTCTAACTCTCCCGAACCCTGCATATCCCTTCTCGTTA
GCATCCCTCCTTTGTTCTCCTCTCCCCTCTGGTCCAGCTCTCTGTTCCTGTGATCCTTTGTATCCATAGCCTGGCCCTTATGTCCTCTGAAAAG
CTGGCCCTTCCTCCATGGGTGTGAACACAGCTCCAGGGGTGTTCACACAGGATGGAGCATCCACCCTGAGCATAGAGGAGGCAGTCAGAGCATT
AAGAGTCCTGCGTCTGGGGCAAAGTTCAAGCTTCAGGCTCTGATTCTTTAAAACTCTTCTGAGATAGGCATTGAGCCCATTTTTTAAAACGTCT
TGGGGCTGAGAATCAAATTCAGTCTAATCTAAAGTGAGACTTTAAGTCACTGACTGAGTCCATCCTGTCATCCAAATGTTGGTAAGAAAGAGTG
ATACTGTCTGTTACCCTTGAACAAAGTAGACTTACCCAGTTAGCATCAAGACGGGCAATTCTCACACATCCTCTCACCTGTACCTGTAGAGGGG
TGTGTGTGCCTGCTAAGCAAAGTCAGGTCCCAGCCCTCCTCTGTTATCTCCCTTCAGGAAGCTCTCNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNTTCTG
TCTCCAAGAATAAACATCATGAGACCTTTTCTATGGCTTGGTATAGTATGGGATGGGGTGGGATGGGATGGGGTGAGATGATATGGTATGGTGT
CATGCTGTGTCATTCTCTATCATATCATATAAAATCATATAATACATAGTATTCTATTCCTTATATTTAGTACAGTTTATTACATTATCACTAC
TTGTCTATCCTGCTAGCATCTTAAAAGATGCAGAAGTCAAATGTGATGCCATTTAGAGACACTGTTTCTGCCTATGTGCTCTATAGGGTAAATA
AATACAATTCTGCCAGGCTCTGGTAAAGAAAGTCAAAGAATCAGACAGCAATTTCCAAGCAGCTAACAACAAGAATGTGTTTTCACAATTAGGA
TCTGTACTTGGAGTGGTCTAACATAACACAGTGTAATTACCTTCATAAAAATCCCCTTTGAAGACTTSGGGATAGATCAGTTAGTAAAATGACT
GTCTAGCTTGCTTGAGCCCTCGATAAACCAGCGCTGCATAACCCACGTATATCAGTAGGTACCTGCAATGCCAGCAGCACTCTACAACTAGAGG
TAGGAGGAGCAGAAGTTCAAGGTCAAGGCCATCCTCAGTGACATAGAAAATTTGAGGCCAGTTGGAGTTACACGAGATTTATTTAAATACAAAC
AAACAAATGGACTTCTGTGAAGATACCTCAGAGCAGTGGAGCAATGACAAGCCCTCCTCCAGACAGATACTCCCTGAGAGAACTTCAATGTACA
CAAAGACAGAGTCCAATTCTAACCCCAGGTCTGAAAGACAGAAGCAAAGGATCACCTCTCACAGAGACATGCCCTCCTAGCCTGGGTCTGAGGT
GAGTCACAGCAAAGACCATATTTACAAGCCTATGGGTTTGCAGATGACAATGTGCAGAGAGAAAGACACTTTGGAACATTCAGGCCTAAATTCA
ATGTTATTGTCATTGTTGTTGTTGTTGTTGTTGTTAGTCAAAAGTCCTCCTTTCATGACTCAGAGATGTATGAAAGAAGAAGTGGAAAGACTCT
AAGAGCCAGGAACACTGATGACTCCAGAGAAACAGTGTCTTGCAGACACACACACACACAGCTGATGCACGTGTGAACTTAAGAGACTAGGACA
GTACACATAGACCTGTACAAGTTCAAGCCAGACACAATCCCAGGGCAGAGACAAGGAAGTAGCTACAAGGACCCATTTCTAACACAGAAGCTAT
TTGCTGTTAATACCTCCTTGGGAGACGGAAAATTAGTTTATACCACTGGAATCCACTGGGTATATTAACCACGCTCCAGGGCAGGTCTCATGCT
CAGGAGCAGGTGGCCAACACAAAATAGACTCCATGATTTGTGTGTGGGTCACTGAATTTTTTGGTTTGTTTTGTCTTTCTGGTTTGTTTTGCTT
TTTTTTTAAGAGAGGAAGAATATGAATTCGGGTAGGTGGGTGTAGTAAGATCTAGAATCAGTTAGGGGAGGGGAATGAATAGGATCAAAACAGA
TCATATGAAAAAAACTAAGAGGCCCATAAATAAAACCAAAACCCACGGTTCAGGGGCTGGAGCAAAGGCACAATAGTGAAAGGCACTTACTACT
CTTTTTTTTTTTTTTTTTGTCTTTTTTTGATTTTTTTATTTTAATTAGGTATTTTCTTCATTTACATTTCCAATGCTATCCCAAAAGTCCCCCA
TCCCCTCCCCCACTTACTACTCTTGCAAAGGACCTGTGTTTGGTTCCCAACACCCACATAGTGGCTCAGAACTACTCCTAACTCCAGTTCCAGG
GGATCCAACACCCTCTCCTGACCTCTGGGGGCATCAAGCATGCACAAGCTACATACACATACATGCAAGCAAACAAGGAAAACTGTGGAGCAAC
CAGCCACAGTCACATACATAATGAGGCCACGTACCCTTGCCTCAGAGACAGAACCATGCTCAGCAACTGGGCCATAATCTGACCCCGTTTCACC
CTCTGTCTCTTCACTCTCCTCTAGAGCCCAGTGCCCCATGGATGCTGGCTTTTTCTTACAGAGCCAGCAACTTGACTTGGATACAGACCATGGG
GGGGGGAGCTCATCTTGGACATTGCAAGGAGGATAGTGGTGTCCTAGCACTGAAGGGGCAATCTCTAGATGGGAGAGATTTTTAAAGTCACAAC
ATATTTATATGTTCATTTATTTGCTGCCTTGGTTACTGTCCATGTACTAGACATGCGAGCTCATGAACACAAGGACCTTGCCTGTCTTGTTCTC
TCCAAGACTCTCAGTAACAAGAACAGCATATGACACTAGGGAGNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNGCACGGCAGGAGTGATGAGAGTATAATGTGAGAAGTCAGGTACC
CAGCAGACCACGGCTGGAGGCCAACCAGACAGGAGGGTCCATCCATGAGAGTACACAATGCTATGATATCTAACTCTCATTTTCTAGGTAATTG
GAGGGGAGGGTGGTTCCTAAAGTTTGGAAACAGAGAAAGAACACAGTAGTGCAACCATGCAGTGGTGTAGGGGGTGGGGGGCGGTCCAGAGAAA
GGAGATGTCAAAATTAGGGAATGCAGTCTACCCCATAGATCCAGGTGGGGGATGATGGGAATACACACGGGTTTGTGGCTTTGGAGACAGAAAG
GAAGTGACAATACAAGGGAATGATGGAGAGAAATGTCTCTACTAGATGCCAAGTGTAGATACTCAGCGGACCCCTGTGGGTCATGTTGAAGCCA
TGAAAGAAAAACGAAACCACCTCACTTAAGCCTGGATCATATAGAAAAGCCAATGACAATTAAACCATTCCTCCAAGAAAAAAAACATATCCAG
AAAGTAGGGCTGCGGTTGCAAAGGAGTCCCTCGGGTGAGCAAAAGGTCTGTGGACCTTCGTTAAGAACCCGATTGAGAAGAGGAAGAAAGACTG
AGTGGATCCTGGCTGCCACAGAGACATGGGCTGTACCAGTGAGAAGGGAAGAAGCAGCAGGAAAGGGGGCAAGATGGGGTGGGAAGAAGGAGAC
AGTTGGTGCCCAGTTGGAAGTGCTTCATATGCACAGGAGACTGTGCACAGGACAGGAGGTGAAGAGGGAAAGGAAGCTGACATGAGTTTTAGAA
GGTTAGGGGAGGCAGGTTGCTGAGGGAGGAGTAGGTGAAGCTCACGGGAGGTCTCAGGTGAGTACTATCTGGATCTATCACAAGTATTCCATCA
TTTTGGGAAAAGTAAGGGGGGCTCTCACCTCATCCAAAACTGCTCCCAGTGACGCCACCCTGCATACCGTCTTCATCTCTATGTTTGCCTGTAT
CTGTTCTGCCCTCATCCTGCATGACCCCGAGGTGACATCACTAATGCTCGGGAGTATCCTCAGGAGAAACGTCTTTTCAGGCTCTGTGACAAGA
ACCTCGGATCTTTTAAACATTTTTTTTCTGTTTCTTTTTGTTTTCTTCCTTTTTTGGGGGGTGGGGGTGGGGGTGTCTTTCATATGTAAGGAAC
TGAACTATGTAGACCTTCCTCTTGGGCTTGGCTGACCCCTTTGAAGCAAGTGTCTACTACATTCTGTTTTCCAAATGTGTAAGCATACATTGGT
ATGTAATTTTAGACTGCTCTCCACTTCTGGCCCCATTTTATCGGCCTTGTTTTGTTGCTGTTGTTTCTCATACTTATTTTTGTAGGTTGTGTAT
TCTTGTAAGTGATCCTGAATTCTTTTCTAGGACAACATTGCACATAAACATGAGGAAATGCACGCGTTATAGCAGAAGGAGGTCTTAAAGTCCA
GAACGTTGTGGCTGGGAAGATCACGGGATGTGATAGCAATAACGAGGGTGTTATTAGGCCAGTCCAGCAGGGACTTAGAGACGCCAGGCAGAGC
TCAGTCTGGGCCACCTCTCTTTTCGGTTTACTGGCTGTGAGAGCCTGGTGTTCTCTAAATCTTACGCAGTGCCCTCTTTGCGTTTGGAGGGGTG
GCTTAACGAGGTAACATGTAGGAAACAGCACTGGATCAGCATTAGCTGTTGGTATTACCGTGGCCATGTTCCAGACAAGTAGCAGGTAGGTCTC
CCCTCTTGGCCAGGTCTGCTGGCACGCTGACATTGCAAGAAAGCCCCTCCCCTAGCCTTCCACTCCCACTTGGCTCTCTTGGTCTACCACACCC
ACAAACTCCCAAGCAGCAGCCTCTAACCTGACAGCTCACCTCCACACCCACCGCCTCCTCCGTAGTGCAGTGCACACAGCTAAGTCTTCTCTTT
TTTTAAATATGCCAAAGAGAACTAAGTGCGTAGTAGTTTAGGGGGGTTCTGCCTTCCTTGCCCAGACCCGACTGTGTACCCTCACTGATACATA
CAAGACAGTCCTGCCATCCGGGAAAGGGTCTCAGCAGAGCCCCCACCCCCACCCCCCTAGGGGACTCGTAGCCTTGGGTGGAGGCAACTCTTCG
CAAGGCTCTAGCTTTGTCAATTTCGGGGCAACTCACTCTGCACCCCTCGGACCTAACTCTTGGGCTGGGAGAGATGAATGGCAGGCGGGGATGA
AGGGAGACGCTAGGGCGGCGCAACTCCTCGGGGCCCTACCAAGTAATGGGGGCAGGATTCGAAGAAGAACTGAGGGCGCCCCACGTTTCCTCCA
GCCTGATGATAACATCAACGCCTGTCACGTGGTTGGTGCGAGAGAGTGTGTGCGCGCGCGGGAGCGAGCGTAGGGAGCGCGCTCGCACAAACTT
TGTGCCTGCGGGTGAGCCTGGGTCGGCTCGCCGGGCTCCAAGAGTGGTCGGCTCCGGGCTGCGAGGGCCAGCGGCGGCCGAGGGATGCGCCGCG
CCCGGCCGAGCCGATCCCACCGCTCTAGGTGAGACGGCTCCAGGGGCGCAGAGAGCCGCGACCATGGGAACCCTGGGCAAGGCGAGAGAGGCTC
CGCGGTGAGTAGCTGCGTCTCCTCGGAGAGCGCTGCACCTCTACTGGGGAAGGATAGCTGAGCGGTGTGCCCTGTGGTCCTCTCCAGTGGGTAC
CAGGAGGGACTAGACGACCAGGGCGCCTCCCTCGGAGGAGAGTTGCCTACAGGGGTAGACCATGATCGGGGACCTGGGAGCCAGGTCAAGATAG
GCGTGATACAGACAACCTCAGAGTCCAGCACACCAGGTCGGGAAGAAAGAGTTGGGGTTGCTGGGGTGGCGAGGCCACAGGTCACCTGGGCAGA
TCTGGAGTGGTCTGAGCCCAGCCAAACCTTTCACCTACCCAGCCCCCACAACAGTCGACCTCCCTCGATGCTCTCGGTCTGGCCACCCCCCGGG
AACTCCCACTCGCACACTTTCCGTGCCCTTGGCACCCGCTGCGGGCGCGGGTCTAGGACAGCGCCACCTTGCGGACTCTCCTGGCCTTTCAGGA
GCGACTGGCGGCAATGGCTGCAAGCGGAGGTGGCAGTGCTTGGCTGCCCTTTGAGGAAAAAGAAGTTTCCAAATCAATGGTCTATGAGTCTACA
CCCCTCCCCTTATACTCTACAGAGATGTTTGGGAGACGGAAGGTGAAGGAAGAATGGCTATGAACAGTCTATCTCTTGGGCAGCAATAAGGAGC
CTGGGGTGGGGGTTGGAGGGATGTCAAGAAATTTAGTGTTTGTCCTCAAAACACCAGGACCCCGTAACTGCCTGGACACCACTTAGCCTTTGTT
AGGGCCGAGAACGTTGTGTCCTTCACAACGCCATTCACGTGGACTCCGATATGACGGCACCATGCCTGATCTGTAACTATGCCCATTAAAATTC
CCTGTTGGGCCATATCATCCCCTGAGTGTTCTCAACCTTACCTTGCGCTACACTATAATGGGTTACTCCATTGAATTAACTCTGCATTATTGGC
GGTGTGGGGCAGGACTGGAAGCTACACTCAGCAGAAATGGCCCTGTGAAAGCTGCCTTTTCTTATAAACAACTATGAGATGTCCCGGCCCTCCT
CTGTTTCCTCCTTTCCCCTGTGGATAACTGTGCCTGGAGACTAAGTCCCCCAAATCGGTTCATACTTCGAAACCTCTCCCCAGGCTCTGTGGGG
TGGGGAAGGCAGACGCTAACTACTGCAGGGCTGAGAAAACATCTTGAAGTTACTGGGTTTCTTTCAAGCTTTTGTTTTTGCTGTTATTGTTGTT
ATTTTGTTTTATTTAATTCATAAGTTTAACTGGAAACGTGTGGCTACTGCCCTTTAAAATCATGACTTTGTAGAATTAGAGGCTGTTCACTCTT
CAGATGGTTAAAGCACTCTCAAAGTTGCCTATCATAAAGCACTGTAAAAATCCTTACCTACAACCATCTCACCAAGAGGTGGAGGGAGGGGAGA
GGGCAGAGGACGTGAAGCGGGGATCTAGCTTGGTGAAGAGTGTTTGCCTGGCATGCAACAATCCTGACTTTGGTCCCTAGCATCATGTAAGAAA
GAAATTAAGATATTTCTTTTAATTCCTTTGGTTGCATCTTGGTGGAGTGATAGGATTGTTCGTTTTCAATCAGGTATGATGGGGGCATATTTGC
AATCCCAGCACTGAATAGAAAAGACTAATACAGAGTTTCAAGCCACATAGTAAGACCCCTGTCTCACAAAAAGAAAAACAAAACAAACAAACAA
ACAAAACCAAAACAAGCTAAAAAAAATTCTTTTTAAAAAAAATGTAAAGAAAGAAAGAAAAAGAGAATGTGACCTGAGAGTTTTTCCAGGGCAA
GCAGTCAAGAGACTAGACAAAGCTTACATGTGCACAGAGTGCTGTTGGCTTTTAACACTTTGCCTTCCCTAGTTTCTCATGCCCTCTGAATTGC
TCCTCACTGGGCTAGGTTTAGGTAGTTGTCTTTTTTCTAGGAAGGGAAGGGAGCCAAGCTAGGAAGTGTGGCTTGGTGCTAAGGAAGCTGGCTG
CTAACCTGCTGTGCATCTGTGGCCAATATATCTCTATTGCATAGCTGTAGGTGTTTGGGAAAGGACTGTGGAGAGACGACTAGCCAGCTCTTGG
GTCCTGGTGACCATTTCAGGAACCTACTAAACAAGGAAGAGCTGAGACGCTGCCTCTGCATCACCAGCCATCTTTCTGAAGCACACGGTTGTTA
TACATTGAATAAGCCCTTTTGGGCTCTTGACAATCTACTCACCAATTTCAGTGCAGGAATAAACACACAGTTTCATGTAAAAAAAAATAATAAT
AAAGCCTACTAGATGAATAAAAGCAAGAGGGACCCCAGATACTAGTCCCTGAGATAGCCCATATGAACAGCCTCAGTATGTGTAGGACTCAGTT
TCATATCACAAGGTCGTATCTTTCTAGACTGTAGCTTCAGACACAATCCAAATTCCTGCACAGTATCTTTGGAACATGGAAACTTCATGCAGAC
TGTTGCTGTTTTCAGGTTATCAGTTTATTCTCAGACACTGATCTGACAGTCTCAGGGTCTGGATAAAAGACGGTGGTGGTCAAGAGTTTAGTGG
ATTTCTGCAGCACCAACCTGATTCTTTGGGAGTCGTTGGCTTGGGTTAGCATGTTCCAAGTCACCAGGGTGACTGCGCTTTATTGGTCCTAGCT
TGGAACTGAGCTCCATGCACACCACAGCCCGATGCTCTGAAGTTGGAGCAGAACTGGAAGGGCCAAGGTGACTCCTGGAGTCTGGCTGTGTGGT
GTGTCCTGTGTTCAGGGTACTGTCTTTGTGCAGGCTGATTTCTACAAACCCAGGTGTGCCCAGGGTGGGGGCGGGGTGGATTTCTGTTTGTTTG
TTTGTTTGCTTGCTTGCTTGCTTGCTTGCTTGCTTGCTTGTTTTAACTAAGGATTTGTTTGGCCTCTTCTGAGTGAGGTTTAGACCTTGTCAGA
GGAGCTCTTCCACCCAGCCCCTGGAAGTGCTTTGCCCTCTGCTTTGCCCTCGACAATCTTGAGACATTCACCTAGAAGTTACGTGGTTACACTC
GCTGTCTTGGTCAGATTCCCCTACTAAAGCTTTGTGTATTAGGGAACTATGAGATGGCTTTATACAAGAGTAACTAACACGCTATGGACATGTA
TAATTTACAGAGAAAGTTACAGAAGCCATCTCACCACGGTCTCCCCCCTGGCCCACTGTAATATCTTACTCATGCGAATAAGTATGCGGCGAAT
CGATGCAGACTTGTGTTTCTAGTATGTGCTATGTTTCTAGACTGTTAACTCCTTCTCTTCTTTGACAGTTAAACTATACATTAGCCAAGGGTGC
AATGAGAAAAGAACTAAGAACATGCAAAAAAAGCTACCAGCAAAAATGCCTATCATTATTAAACCTTCAAACATTATAGACGTGGGTCTCTGTG
TGGTTTTTAATAAAGAGGAAAATAAAACATACTGCATGAGACACTTTGCATGGTGACCTGTATCAGCCAGATCACGCCAAGCTAAGGATGAGCA
GACGAAGCGCCACCAGACTCCTAAGCCATTTCCAGATCCAAAATGGTTACTTTATATTCGGTTCTAGAGCAGGCAGCTCAGCTCAGAGCCAACC
CCTCCCCAGTTTCTCTTCGTTGAGGATCAATGTAAAGACCCGATGGAATACTGTTTACTGGTCTAAGTGATTCATAGTGGTTCCTTGTGTTATC
AAAAATGAAACCATTTATTAAAACCAACCAAGATGTGTAAGAAATGCAGAGATGACTGAAAAATCTAAATGTCACCCCCCTGCTTCTTCAAGAT
TCTCAAAGCTAGTTCAGGGGACCTCCTGCCCTTCTGTATCTTTGGCATTGCTTAAATGAATGAAACTATCCAAAAAGGATGCTAACTGAACCAG
TCCTTCAGACCCTGGGTCCGTAGCCTAGCAACTAGAAGCTAAGGGCTCCCAAGGAAGCTTCTTCCCTCTGCATCTCCCCTTCTGGAAGAGAGAC
GGCAGCATAGAGCAGTCCTTACACCAATGGGCAGAAGGCTATTGCTGCCCTGGAATCTCACCTTGCCATTTGCTGGGGAGATGTCTCTTTAACA
GCCAACCAGTCTGTTCAGTGCTTTTGCCAAGTGGGAGGGGTATGCCTTCTCCCAGCCCAGCTTCAGAGACTCACTGTCCTATCTCCTCTTTGCT
TGGTCTTCGGAGCCTGGACCTGCCATTGAGACCCCCAGCTGCTCTCTCTATCCTCACTTGAGTCTCTCTTTCCCCCAGGAAACCTTGCCATGGC
TCCAGAGCTGGCCCCAAAGCAAGACTAGAGGCCAAATCAACCAACAGCCCTCTCCCTGCCCAGCCCAGCTTCGCCCAGATCACCCAGTTCCGAA
TGATGGTGTCCCTGGGACATTTGGCCAAAGGAGCCAGCCTGGATGATCTTATTGACAGCTGCATTCAATCTTTTCGTGAGTTGGGCAACTGCCC
ACCTATATAGAGATTGCTGTGTTACTTTGTTCTGTGCCTTAAAAAGTGTCTTATACAAGAGCAACCTAGATTGTATTTTGCCTTTAAAAATAGC
TTTAGAGCTTTTGAAATGTTGGAGACAGTCTGTAGCGCTTACGGCACATGACTATGTGGGTGGAATAGGTCGTGATGAGAAGTACAATTTGAAG
TGAGAACTTAGTGAGCGAAGTTGGCATTTTCCTCGCATGGTGGTTTGTCTCATGGTGAGGGGAAAAAAATCACAAATTGATATTGGGTATTCTT
CTCCTCTAGTTCTTGTGGTCGAAAAATCCTAAGCCTGTCTCACACCGCCAAATACAATTTGAAAACTTTTAGGTCATTTGGACAAATAAGGTCT
CGACAGTTCCCCCTTACAATCTGGACTTGTGCTGTGTTTGTGACTAGAAGATGAGGAGCCCATGTGTTGCAGGTCGAGGTGGGAGGACCTGAGC
ACATGGGCACATAGGCCGCCAGCGGTGTATGGTCCAGGTGCTCCCCTCTGTACTGCCAACAAGCAGTGCCCTCAAAAGATTTCTTTTTATCCAA
ACATTTTCTTTTGATCGTTGCTTATGAAATGGTGACTTGAAAGTAGTACTAAATATTGGGTTTGCTTCCAAAAGGGCAGGATTAAAACCGACAG
ATTTCACTGAAACCCATTAATTCAGAGATGTTTCAGTGTCCCCACGTCCCCTTTTCTATGCTCATGCATCTCCCTCTGGGAAGGAGATGGATTG
GTGGGACTCGGCTCTGAAGCATCAGCCCACCCCAGCCTGATCCTCCTGGACAGCAGAGGGGAGTTGTGCTCAGCTCTCAGAAGTGGGGACTGTT
GGTGTTTAGGCCCAAACAAGGACCTTATTTATAGACTTGGGGAAGACTTGGCACTTTGGGGAGATTCTTTTTGTGCAACTGCCTCAGTTTTGGT
TTAAAACAGTGCTTCTTAAACATTCTTGCACACACAGTTTGGGGGTTTTGATTTTTTTTTTTTTTTTTTTAGTAATGCAGATGGCTAGGTCCCA
CCCCCAGAGTTTCTGCCTGAGTAGGTCTGGCCAGGGTGGGGTCCAAAAATTTGCATTTCTAACGAGGTCCCAGGTGAGGCCTTGACAGCTGCTC
CCAGAACTCCACTTTGAGAATCGCTACTTTTAAGTTCTGTTGGCTGTAGACTTCTTGACTTTGAAAGAAAGCAGATTGGGTGTGTGACTCAGGC
TCAGTGTTTACCTAACAGGCAGGGGCTTGAGTGCAGGCTCCATCCCTGAAAAAGAAAAGGCCATTCATTCTGCTCCCTGTTTTATCTTTACCCG
AAAGACTAATTCAAGATGAATTTTCTACTGCTAAGCTACATCTTCACCTCCAGATTGAATAACTTTGTGTCTCAAAATCATCATAGACGATTTT
TTACATTCAAGGAGACAGCTAGAAGCTGGTGGAGACCAGTGGCCATGTGGTAGTCATTAGTGTTCTTAGATAAGCTCCTTGACTGCTAATTGTA
TTCAGTAGACCTGCAGAGAGCATAGTGTAACTATTGCATGAACAAGGACCTTTACTCTCTCTTTCACTGCTGAGAACGCTGGGGCCATCACCGT
GTTTATCATGTGAACGCTCTTCTTTTTCTACCAATCATCCAACTACCTGATGAGTTCAGCCTGTTTTCTTACCCTTCAAATACCTGTAGCTTCA
ACCTCAAAAATACATGGCCAGTTCTGGAGTCCTTTCCAGAAACAACCAATCAGAACCCACTCCCCCAACCAGCCCTACAAAGGCAATGGTGCAT
TGTTTCCTGCAATATGTGAAGGTAAAAAGTGGTGCTTTAAAAAACAGGAGCTGAGTAAGTGCCACTGAGACTAAAGCTCCTCAGGCCACTACCA
AGAAGCATTTATTAAGTCTGTAATTATGCCTGACCCTGCACTGGGTGGCACCCAAGAAAGTCACAGTGACCAAGTGCCTGTTTCATTAGCACTT
ATGACACCTGTGAAATACCCGGTTCAAGAACACACACACACACAGAGACTCACACATGCACATACCTGTGATAATAAGTTAAGGTGTGGTGTTT
GGAAAACACACACACTCACACACACAGTCACACACTCACACACATACACTCACACACACATACACTCACACACACATGCACACACACTTGATAA
TAAGTGAAGGTGTGGTGTTCAAGAACACATTCACACACACACACACACACACACACACACACACACACACACACTAACAAATGAAGGTGTGGTG
TTCAGGAGCAAGTTCCTTGTACTAGGTAGGGTTTGAATCAGGGCACTTATTGATAAGGACCAGTCATCACCTCACATACCCTGGGCCATAGGCA
TGCTGTGGACAAAACCCCGAGCTTTCTGTATTATATGTTACAGAATTTCCCATCACAGAGTTTACATGAAAGACACAAAACAGCCAAAGCAGGG
AAGAATTAGAAGGCTGCCTTTGTGAGTGGCCTCCTCTTGTTATCAGCTGACCTAGCTCTTGTGGGAGGAGCTACACTCTGAGCCGTGAAAAGCC
GGTTTGAAGATTAAGGCAACTTGACTAATTCCTTTCTTTACTTTTTCCTACCTGCTCATTTCACCTTCATGATGATGCCTAGGAAGTATTTCAG
GAGGCTGTGTACACAGCACCCACAGGGCCCCCCCCCCCCCGAACACAGGGGCCATTGTCATACACATTTTAATTAGTTAAAACAAAACCAAGTG
TAAAGTTTAGTTCATGCCACAGCAGCCACCCTTCCGAGTGCTTGATGGCCTCAGGTGGTGACTCCTGTGAAACCGAGTAAGCAGACAATGCCTG
TGGTCACAGAGTCTGACTTGGGTACTTGAGAGGGAAGACAACTGACCTGAGGACACAAACGGTAGCTAGATTTTTCTGGAAACTGAGGTTTTAT
TCATGTCTCAGGTACACCTAAGGGACAATCTAGTTACTGTTTAAAACCTCATGGGCATTGAGACGTGTATACTGAAAGCTGTCTCTTGAGCATA
ATTAAGTGTTAGTTGTCCATCCCTTCCAGCAACTGCTCTCTTGGAAGGCTGGAATAGAAGGCCTTTAAGGATACGAATGCAAATGAAGGTACTG
TGGACGGCCCCCAGCAAACCTCTCTGTATCAGAAGGTTACAGATCTGTGATTAGAAACACCGAGCCTCCATTCCGTGGCTTTACCCCTGCACCA
GCCCACTCCCACCTTCTGTTTTAATCCGAGGGCATGCTACCCATCCCGACTGTGGCTGCTAGTTAACGCTATCTGAGGTAGATCATCACAGCAG
GTGAAACATACCGTCCGCTAACAGGTTAAAGGCCTATGAGAAGCACTTAGGCTTTTCGCTTCTTCCTCACTTTGCAAGAGGGCTCTGAGCATTG
GGGCCAAGTGGAAAAATGGAATACCCCAAGTTACAAGGTGCTTTCATGTCTCCTCTCTGGGCAAGAGAATTCTGATAGCATCAGGGAACAGTTG
CACTCGTACCTCAGAAAGCACGGTAGCAAGCAGTTCCTCTGTGTCCAGATCGGGATGTCAGGGCCGAGGACAGTGCATCCAAGTGGCTTATATT
ATATGGAGTGTTACTTTTTTTTCTTGGTGTTCTGTATGTATTAATTTTTCTTCTATATTCAAAACAACCCTGAAGTAGGTCAAGTATTATTATC
TCCATTGGGTGAACAAGAAGAAAAGTGGGACATCTGAGATCTGAAACAACACACCCTGTACCCACCATCTAAATAGAAAACAGGCTGCCTTTTT
CTTCTGCCTCTACCACCAGCAGCTATTTGTACCCTGAGCTCGTGCAGATGTGCCTCACTTCCCAAGACAGGCCTCATAGCCTCAGAAGAGATTT
CAGATTTTGTGTTGCACAAGGTAGGCTGGCAACCTGTGCTTGCTAGTCTGCAGGCCTGGTCTCCCTCAGTTAGAAACCACATTCCGTTCAGTGC
AAGAAATAGTCAGACTTCACCTTTATACCATCAACTGTCTGGTTGAAGTTAATCGAGTCAAACTTATATTTGTAAGTTTCTATGTGTATGCATT
GTACTAATGTTTCCCAAACTAGAAAGACCCTAAAACCAGTAAGAGTAGTTTCTGTACTATGGGCTAGTTGAATACTAAGCTCCTTATAGACCTT
AAGGAATTATAGTCTTTGAGACTGGAGCTGAGGAAGGGCAAACACGTGCTTGCTGGAACCGCTGTCCCCTCCACAGCAAAGTGAAGGTTGGATG
TTCCTCATCTCAGCTTTAACTCTGCACACTGAGGCAAGGTGTACAGGAGACAGGCACCACACCACGCTCTATGACCCAGATGGGTCCTTAGCAC
TGACACCTGCAGGCACCAAGCCACATATATAAGGTCACATATACAAAGCCTTACCTAGGGCTGATAACAACCCCCGAGGGTAAGCCACAGCTGT
CCCTGAAGCCTCCTCATGGAATTCCCCAGATTTACAAGGTTTCTCGTAGAAAACAGCAAACAGCCATTTGCGGGGGGCACCCTACCCTAAACAC
CTGGGCCTGTCAGAAGTGCCCAGTATGTGGAGAGAACTGGCATTCTTCGTTCGGTGTGCCACTTAAGCTAAAGAGCCCTTTACCATGTCTCTCC
TGGGGTGATATCAACCAGTGCCCCTGCAATATCTTCCCTTTCATGTGTGTGAGTTCTGGTGACTCTTGGGTCPGAGGAAGGAGATGGTTTAACC
CTCTTCACCTCAAACTTTCCTGTGTGGCATTATCTCCAGGAGCCCTCCCTGTGTACTGGGGCAAAATCAGGCCTTCAGACGATGATGCCTAAGA
GCCACAATTTCTCAGGAGTTCTGGGTACTTTTCTTCAGTAGGTGGACCCAGAGGAATGAATCTTACTGCTGACTGAAAGACAGAGGGTGTGGAG
TAGCTGAGCACTTATGGCTCGTGGGTGCAGTGGGGGCGTGGGCCTTTCCCTGATCTGACCCTGCTGGGCTTCTGAACAGCACTGTGCCTTGTTA
GTTTCCTCTCTTAGCTGATAGGGAACTGGGACTCAGAAGACGGAAAGGACACATGCCTGGCCCAGCTCATTACTGGCCCAGCTGCTGAGACCAA
AGCTCAGGTCGGCTCTCTGAGGCTTGTGTTCTCTCTGCTGGAATAGAGTCTCATCCTGTGTGAGTTTGTCAGACATGCAGCCCCTCATGAACTG
ATACCGATCACCACTTCCCTGTAGCCCGCTTGCTCCTTGGACATCCCAGAACATGTTCTTAGGCTTTCTAGGAAAACAAAACCAACTACAAACA
GCTCTGAGAAGCAATGGAGCCAGTAGGCTTTTCAATTTATAAACCGATAAAGCTCTCGGGGAAGCCAGAAAGCATGAAAGCGGTCCAGCAAGGT
TTAATTAGAGACACTCTTTGCCTGGGAGCATGTTGGAAATCCCAGCACAAGCTAGCAGCAAAATTTACAACCTGTCAGCCTTACACTTGGGACT
TGGGGATGGGGGCAGTACTTCATTGCTCTCCAAGACTTTTCTTCAGGCTTACATTTGCTGCTGTGGGGAGATGGTCAGATCCATTCCTGATGCT
ACAGTAAGGTCTCCATTCTTGTCTCCAGCCAGCCTCCTGGACTCACAGGCCTTCCAGAAGAGGCTGAGCTGGGAGGAGCCAGAGGGAAGCCCTG
AACAACCAGCCAGATGAACCTGTTTGCCAGTAGATTCTTTAAGGCTTTCATTGGGAAATAATAGGAATTCCACACCTGTCTGTTTTGTTATCAG
AACAGGGGCTGGCCCTCTGAGAGCCTCGGAGAAGGGCATCTTAGGGGCAAACTTACCTCCTGGCCCTCTAATTGTTGCCTCATGCCTGAAGCAC
TTGGTGAAACTAGGTACTTCAATGCCAAACAGTCTTGACATCCTTAATAATCCTTAATAATTTAATTAATTATTAATAATTTAATGATCCTTGC
TGATTCAACTATAATGTTGAAAGGGGGGCATCTTTTCATACCTATTGGATCTTAAAAACCACATGGCCTGTCATGAGCGTGAGAATACTAAGAA
TGGATGGCGGTGGGTGTCTTGCCAAATGCTGACGATGTCGAGGAGATGGGCCTCTAACATATTATATTTTTTATGAACATGGAAGTGTGAGTAT
GAGAGTGGAGGGAGATGATAGCCTGATCTGCCAGCTGTGGCATACTGGATGATTATCCAGCACACAAGATCTAACCTGTTCTATTTTGTCTAAT
TTTGCAAGCCAGTTGTTAAACATAGCCACCATTAAAGAGTCTGTTATTTACATGTGTGCTGAGTAAGTTCATAGAAGACAAGCATTGGAAATTG
GTCACTTTCTAATGCGTGGTCATGTTTACTGTTCTGTGTGCTTAGGAGGGTGTGTCCATCATGCCTGTATGGTGAAAAACACTGCAGAGGCTTT
TGCTGCAGTGTAGTGCAGTGCAGTGCAGAGCAGGGCAGCGCCCCCACTGGTCCATCTGCAGCAAGGCAGGCTGGGAGCTTGGCTTTGCCAGTGT
AGTCACACCTTGGAAATAAGCAACATTACTGCCAGCCCAGCCTCCTCAAACCCAACCCCCTAAGAGCTCGAGGTTAAAGGCTTACCAGCATAAC
ACCATTGTCAGTACTAAACCCCGGGGCGCTGTCAGAGCAGAGTCTAGAGGGAGGGGACCCTCAAAGCTGACAATCTTGTTCTGTATCACTCAGT
TAGGAGTAAGGCTAACATAGTTTTTGGGTCTTTCATACCCTAAGTAACCAACCTCCTTGTCTGTGGTCAAGACAAGACAAGGCAAGATGAAGAA
ATTAAACATCAACAAAGCTGGGATTTTCTTAAAGACGTTATTATGGAACAAAGATAAATCCAAGAGATTTCATTTGTAATTGAACCAATTACAT
ATATATATATATATATATATATATATATATATATATATATACATACATATATATATATATATTTTTTTTCCCCCTTAACTCACCTCCACACATT
CTGCAGGATTAAGCGTCTCTTACTTTTTCAATTTCTTATACTTCTGACCTCCACATGTATGACCCATCTCTATCTGCTTAGGGCTCCGTCTGCA
GTGTTCATTTGCTTTTATTCCCTCTGCGTGTTGACAGCTTCAGTGGAATGAACACACGAGCTTTGCTAAGCAGGTGTTTTTTTTTTTGTTTGTT
TGTTTTTTTTTTTTTTTTGTAATGAGTTTGCTGTGTTCCTGATCATGAACAGTCTCTTCATACCTGTTTGTTATCTGCTGTTTACGGTTCTATA
TTGCTAGAGGTTTGCACCTGTTTTCTTGGCTAGCATTTGTTGATGCCTTTCTCCTATTTTCATCCCTTCATTATTTTTACTCTTACAATACGTA
GAAATTCCATGGTTTTATGTAGTTAAATTTGTCTTAGCCTTTTAACCCTTTATTAATTCTAAACTTAAAATCTATACTTCACCCACTTGGAAAA
TATCTTCCTGTCATTTTAATTAAAATGAAAATTATTTTAATTCATTCAGTGTATATAATATGCATTATACAGTCTTGGGAATGATCAGTAAAGT
TTGCAAGTTATTGCTGGAGGAGAGAAAGCCTTTCGCTTTATTTCTGGGATTAGCTTAAAGCCGGTCTGGGTCCTACTACACACTAGACTACTGC
ACACAGGTCATTTTGGCACTCAGAACCCTGCTCTCTCTGACTCTGTCACAGATGATTAAAATCAGTTCTAAAACCAAGCAGTGTCTGGAGGAAG
AAATAGCGTGATCTCAAATATGATATTGAATGTCAACTTGAAGGAAACCCTTCAGAACTGATGTATAAAGTGCTTTTAACTGTTTATAGAACCA
ATAGCCTTAGAAGACTAAAACTCTCGTCACATTTAAATCTCATTTTTCTAAGTTATTTCTGTAGAGTGAACCTATGTTGCTCCTAAGTAGGCCG
TTTTGTGACGAGATACATAAAACATTAACAGTACTTATCTATTTAAATATTCACTGTTATTTTAGAAATAACCAGCCTTTGGATAATTATTGTG
AACAGTACCGTGTTTATGTTAATATTATATATATTTATTTAATTCACACTCTTATGTTCCTATAACTACAAATATTTAACTAATACTCTAAATT
CATATTTTTAAAAAATTTTAATAACTTTTAAAAACCATTTGCTTCACTCTGGGAAACTTCAGAAATACAGTTTAAAAATGAAAAGCATTGGGGC
TGGACACATGGTTCAGAGGTCAAAAGCACTGGCAGTTCTTCCAGAGGATCCTGGTTCAATTCCCAGCACTTACATGGTGACTCACAACGATCTA
TAATTCCAGTTCCAGTAAATCTGACACCCTCACAAACACACATGCAGGCAAAACACGAATGCATGTAAAACAAAATTAAAAATCATTTTAAAAA
CTTTTTAAAAGGAAAAAAATTACAGTTCTGTATCCTACCCCCACAAACAGTGTTAGCTATGAACATTCTTGGATTTGTCTCAGCGTTCTCCTAT
GGATATGCACGGATACTGTGAACATAGTGCATGTCTTGTGATAGATTCTCCATACCTCTGTGTGTTCAATATTCACATCTTAACCAATTGTGAT
CCTGCGATGAACATTACTACGTGCCAAGAAGCGTGCTAGCATACAAGGTTTCTACTCTATCCTAAAATTGATCCTAAGAAAATAATGCTGGCAT
AATTCCATCAAATATGGGACATAAAATTGAGGGTTGCTTGGCAACCAAAATGACGCTGATGAAGAATTAAAGGCATTTCCAGAGAAGAAGAATA
CTGATTTTTCCAATGTAAAGTGCTAATTATAATACCCTAGGGCTGATTACAGATAGAATGAATGAAAGGGAGCAATACTTAAACTAACTGAAAA
TCCAAGAGGCAAGCATTCTTCTTCGTTATGTGTGGCGAAAGTGAGTTAAAGGTTTACTGTCAGAAACGAAGAATAAAATCTCTTGCCTTCATTA
TAACTTCTCTAAGTTATAATGAAGGAGCAGTGAGGTGGGATTTATAGCAGAGCCTAGAAGAGGCTGAAGTCTGCTCAGTCCAGCTGCTTTTGCA
GTGACTGTGAGGTGGCGGGCAGCATCTGAGGAGGCCGTACTGTTTCCTTCCTTTTTAGTGCAGAACAGGCCAGGGCTCAGGCACTGAGCATCTT
GCTAAATGGTTGTACCGTTTGGCAGAACCAGCTGCCAAGATGGAGCTTGAACAAGGGGCACACCAGTGATTGACTGAAATGAACAAAAGAACAT
AGGTACTACTACAGGAGGAAGAAAGAGCAGAGAGTACTAATTTTATCCTGAAGGAGAACACGGATTCATAGGAGCAAGATGGGTGTTGAGTATA
AAATCATCTGAAGGCTCACATGGAAAGTGAGAATTCCCAAGTAGAAGAGCAGACAGTTCTTCTGTCTCCTCTCACTGATCTGCCGAACTGTAGT
ATGGACTTGTTTATATGATCTTTTGAACAGCCCCATTTTAAAGAGGAACTGAAGCACTCAGGAAACGGGTGTCCCCCTAATTATCATATGTGAG
TGAGTGTACTTGTTAACATATAGTCACACCCTACAGAGCACCCTTGAGTCTGTTGAGCAGTTGGCTAACTCTGCGTTCGTTGCTTCCTGCGTGA
GCATAGGGACTTTTCCATACTTCCTTCCTTTCTTTCACACCTTGGATTCCAACTTTACTTCATCCAAGAGCTTCGCCATTTACCTCCAGGCCTG
GGCCACTCCAACCACACAATAAACCAGTTAGCATCTGATACAGTTCTTTCCAGAGGTCAGCCACAGGACCCACACTGCTGGCTACCCAGCTATC
TTCAGTTCCTGCTAAGGATGAGTCCTCACTCTGTAATAACTTGTGAGTTGGCTCTACTCTATGAACATACTCCCCTTACTTATTGTGCCTATAG
TCTCCCGCACTGCCTGCGTGCCCAATGGCTTAGAGTCCTATTACCTTGTCACCAATCACAGGTAGATTTAGCTAACTGAGGAAGTGGACCTTCA
TAGTGTCACAGGGGAAAATATGAA
MOUSE SEQUENCE - mRNA
GGATCCGCGGCCATCGGAACCCTGGGCAAGGCGAGAGAGGCTCCGCGGAAACCTTGCCATGGCTCCAGAGCTGGCCCCAAAGCAAGACTAGAGG
CCAAATCAACCAACAGCCCTCTCCCTGCCCAGCCCACCTTGGCCCAGATCACCCAGTTCCGAATGATGGTGTCCCTGGGACATTTGGCCAAAGG
AGCCAGCCTGGATGATCTTATTGACAGCTGCATTCAATCTTTTGATGCGGATGGAAACCTGTGTCGGAATAACCAACTGTTACAAGTCATGCTA
ACCATGCACCGGATCATCATCTCCTCAGCCGAGCTGCTACAAAAAGTCATGAATCTATATAAGGATGCCCTGGAAAAGAATTCTCCAGGAGTTT
GCCTGAAGATCTGCTATTTTGTCAGGTATTGGATAACAGAATTCTGGATCATGTTCAAGATGGATGCCAGCTTGACCAGCACCATGGAAGAGTT
CCAAGACCTGGTGAAAGCCAATGGTGAGGAGACCCACTGCCACCTCATCGACACGACCCAAATTAATTCTCGAGACTGGTCCAGGAAACTGACT
CAGAGGATAAAATCGAATACTAGCAAGAAGCGCAAAGTGTCCCTGCTGTTTGACCATCTGGAACCTGAAGAACTGTCTGAACACCTCACCTACC
TTGAGTTCAAGTCCTTCCGACGGATATCTTTCTCTGATTACCAAAATTACCTTGTAAATAGCTGCGTAAAAGAGAACCCCACCATGGAGAGGTC
CATTGCCCTGTGCAATGGCATCTCCCAGTGGGTACAACTCATGGTTCTCAGCCCTCCCACCCCGCAGCTCCGGGCAGAGGTCTTCATCTAGTTC
ATCCATGTGGCTCAGAAACTCCACCAGCTACAGAACTTCAACACGCTGATGGCTGTGATCGGGGGACTGTGTCACAGCTCCATCTCCAGGCTTA
AGGAAACAAGTTCACATGTCCCACATGAGATCAATAAGGTTCTGGGCGAGATGACTGAACTGCTGTCCTCCTGCAGAAACTATGACAACTACAG
GCGAGCCTATGGGGACTGCACCCACTTCAAAATCCCCATACTGGGTGTGCACCTCAAGGACCTCATATCCCTGTATGAGGCTATGCCCGACTAT
CTGGAAGATGGGAAGGTGAATGTCCAAAAGCTCCTGGCCCTTTACAATCATATCAATGAATTGGTCCAGCTGGAGGAGATGGCCCCACCATTGG
ATGCCAACAAGGACTTGGTGCATCTGCTGACGTTATCCCTGGATCTATACTATACAGAAGATGAAATCTATGAGCTTTCCTATGCCCGGGAGCC
GAGGAACCACAGAGCTCCGCCACTGACACCTTCGAAGCCACCAGTTGTAGTAGACTGGGCCTCTGGAGTGTCTCCCAAACCTGACCCAAAGACC
ATCAGCAAACACGTCCAAAGGATGGTGGATTCTGTCTTTAAGAACTATGATCTCGACCAGGATGGCTATATCTCTCAGGAGGAGTTTGAGAAGA
TCGCTGCAAGCTTTCCATTTTCCTTCTGTGTGATGGACAAAGATAGGGAAGGCCTCATCAGCAGAGACGAGATCACGGCCTACTTCATGAGGGC
CAGCTCCATCTATTCCAAGCTGGGCCTGGGCTTTCCACACAACTTTCAAGAAACCACTTACCTGAAGCCCACCTTCTGTGACAACTGTGCTGGC
TTTCTCTGGGGCGTGATCAAGCAAGGCTATCGCTGTAAAGACTGCGGGATGAACTGCCACAAACAGTGCAAAGACCTGGTGGTGTTCGAGTGCA
AGAAGCGAATCAAGAGCCCAGCAATATCCACAGAGAACATCAGCTCTGTGGTACCAATGTCCACCCTTTGTCCACTGGGAACCAAAGATCTGCT
CCATGCACCCGAAGAAGGATCTTTCATTTTCCAAAACGGAGAGATTGTGGACCACAGTGAGGAGAGCAAGGATAGGACCATCATGCTCCTCGGA
GTGTCCTCCCAGAAAATTTCAGTTCGGCTGAAGAGGACTGTTGCCCACAAGAGCACCCAAACAGAATCGTTCCCGTGGGTTGGTGGCGAGACGA
CCCCTGGTCACTTTGTGCTGTCTTCTCCAAGGAAGTCGGCCCAGGGCGCTCTTTATGTGCACAGTCCACCATCTCCATGCCCCACCCCAGCACT
GGTCCGAAAGCGGGCATTCGTCAAGTGGGAGAACAAAGAGTCCCTTATAAAACCAAAACCAGAACTTCACCTCCGGCTCCGGACCTACCAAGAA
CTGGAACAGGAAATAAATACCCTGAAAGCAGATAACGATGCTCTGAAGATCCAACTGAAGTACGCACAGAAGAAAATAGAATCCCTGCAGCTTG
GGAAAAGCAATCATGTCTTAGCCCAGATGGACCACGGTGATAGTGCTTAATCCAGAATTTCAAGGACCAGAATCTGCAGACGGGTTTACTGGGA
TGTCGAC
MOUSE SEQUENCE - CODING
GGATCCGCGGCCATGGGAACCCTGGGCAAGGCGAGAGAGGCTCCGCGGAAACCTTGCCATGGCTCCAGAGCTGGCCCCAAAGCAAGACTAGAGG
CCAAATCAACCAACAGCCCTCTCCCTGCCCAGCCCAGCTTGGCCCAGATCACCCAGTTCCGAATGATGGTGTCCCTGGGACATTTGGCCAAAGG
AGCCAGCCTGGATGATCTTATTGACAGCTGCATTCAATCTTTTGATGCGGATGGAAACCTGTGTCGGAATAACCAACTGTTACAAGTCATGCTA
ACCATGCACCGGATCATCATCTCCTCAGCCGAGCTGCTACAAAAAGTCATGAATCTATATAAGGATGCCCTGGAAAAGAATTCTCCAGGAGTTT
GCCTGAAGATCTGCTATTTTGTCAGGTATTGGATAACAGAATTCTGGATCATGTTCAAGATGGATGCCAGCTTGACCAGCACCATGGAAGAGTT
CCAAGACCTGGTGAAAGCCAATGGTGAGGAGACCCACTGCCACCTCATCGACACGACCCAAATTAATTCTCGAGACTGGTCCAGGAAACTGACT
CAGAGGATAAAATCGAATACTAGCAAGAAGCGCAAAGTGTCCCTGCTGTTTGACCATCTGGAACCTGAAGAACTGTCTGAACACCTCACCTACC
TTGAGTTCAAGTCCTTCCGACGGATATCTTTCTCTGATTACCAAAATTACCTTGTAAATAGCTGCGTAAAAGAGAACCCCACCATGGAGAGGTC
CATTGCCCTGTGCAATGGCATCTCCCAGTGGGTACAACTGATGGTTCTCAGCCGTCCCACCCCGCAGCTCCGGGCAGAGGTCTTCATCAAGTTC
ATCCATGTGGCTCAGAAACTCCACCAGCTACAGAACTTCAACACGCTGATGGCTGTGATCGGGGGACTGTGTCACAGCTCCATCTCCAGGCTTA
AGGAAACAAGTTCACATGTCCCACATGAGATCAATAAGGTTCTGGGCGAGATGACTGAACTGCTGTCCTCCTGCAGAAACTATGACAACTACAG
GCGAGCCTATGGGGAGTGCACCCACTTCAAAATCCCCATACTGGGTGTGCACCTCAAGGACCTCATATCCCTGTATGAGGCTATGCCCGACTAT
CTGGAAGATGGGAAGGTGAATCTCCAAAAGCTCCTGGCCCTTTACAATCATATCAATGAATTGGTCCACCTGCAGGAGATGGCCCCACCATTGG
ATGCCAACAAGGACTTGGTGCATCTGCTGACGTTATCCCTGGATCTATACTATACAGAAGATGAAATCTATGAGCTTTCCTATGCCCGGGAGCC
GAGGAACCACAGAGCTCCGCCACTGACACCTTCGAAGCCACCAGTTGTAGTAGACTGGGCCTCTGGAGTGTCTCCCAAACCTGACCCAAAGACC
ATCAGCAAACACGTCCAAAGGATGGTGGATTCTGTCTTTAAGAACTATGATCTCGACCAGGATGGCTATATCTCTCAGGAGGAGTTTGAGAAGA
TCGCTGCAAGCTTTCCATTTTCCTTCTGTGTGATGGACAAAGATAGGGAAGGCCTCATCAGCAGAGACGAGATCACGGCCTACTTCATGAGGGC
CAGCTCCATCTATTCCAAGCTGGGCCTGGGCTTTCCACACAACTTTCAAGAAACCACTTACCTGAAGCCCACCTTCTGTGACAACTGTGCTGGC
TTTCTCTGGGGCGTGATCAAGCAAGGCTATCGCTGTAAAGACTGCGGGATGAACTGCCACAAACAGTGCAAAGACCTGGTGGTGTTCGAGTGCA
AGAAGCGAATCAAGAGCCCAGCAATATCCACAGAGAACATCAGCTCTGTGGTACCAATGTCCACCCTTTGTCCACTGGGAACCAAAGATCTGCT
CCATGCACCCGAAGAAGGATCTTTCATTTTCCAAAACGGAGAGATTGTGGACCACAGTGAGGAGAGCAAGGATAGGACCATCATGCTCCTCGGA
GTGTCCTCCCAGAAAATTTCAGTTCGGCTGAAGAGGACTGTTGCCCACAAGAGCACCCAAACAGAATCGTTCCCGTGGGTTGGTGGCGAGACGA
CCCCTGGTCACTTTGTGCTGTCTTCTCCAAGGAAGTCGGCGCAGGGCGCTCTTTATGTGCACAGTCCAGCATCTCCATGCCCCAGCCCAGCACT
GGTCCGAAAGCGGGCATTCGTCAAGTGGGAGAACAAAGAGTCCCTTATAAAACCAAAACCAGAACTTCACCTCCGGCTCCGGACCTACCAAGAA
CTGGAACAGGAAATAAATACCCTGAAAGCAGATAACGATGCTCTGAAGATCCAACTGAAGTACGCACAGAAGAAAATAGAATCCCTGCAGCTTG
GGAAAAGCAATCATGTCTTAGCCCAGATGGACCACGGTGATAGTGCTTAATCCAGAATTTCAAGGACCAGAATCTGCAGACGGGTTTACTGGGA
TGTCGAC
HUMAN SEQUENCE - GENOMIC
GGTGCTTGGGAGGGCATGGCTGGAGAGGTAAGATCCAGTCATGCAGGTACATGATGTTAAAAAGTTTGCACTTTATTCTGTAGGCAATTGGAGA
CTCCTAGGCAGTTTAGAAGCAAAAGAATTATGTGACCATGTACATGAAAGAAGCAACATGAAAGAAGAATCAGAGAAAAGGAGGTTATGTAATT
AGGGACGCCAGTTACAAAGCTATTACACATGTCCAGGTAAGAGATGATGGGGGCTTAGACCAGCATGTGGCTTTAAAGATGGAGAGGAAGCCCT
TGGGAACAATGAAAGGAGGTGGTGGTTGAAAATGTGGGTCTAAATGCTAGGTGACATAGGTGGATATTCGGACAGAAGAGTCATATAGGTCCTG
CTGAGGCCATGGGAGAAAATTAAAACACCTACTTTGAGTAACGGATCATTTAGAAAATCTGAAGAAAGCTAACAACCCTTCCCCCAGAAAAAAC
ATACACAAAAATTTAGATATAGTTTCAGAAGCTTCACATATAGTATAGTCCCTTCTCCCCAGTTAATCTCCTCATGGACATTAATTAAGAGTCC
ATGATTGAGAGAAGGAAGAATAACAAAGAAAGCATCCAGAGGAAGGGCCTCACCCAAGGGGTGAGAAGAGGAAGACTCCCCAACCAGACACCTA
GGAAAAATAGAAGAGAGGGTAAATGAGGGTACAAGATAACACACTTGGGTGGCTGGCAAGATGGCCAAATAGGAACAGCTCTGGTCTGCAGCTC
CCAGCGAGATCAACACAGAAGGCACATGATTTCCGCATTTCCAACTGAGGAAACTGGCTCATCTCATTGGGACTGGTTAGACAGTGGGTGCAGC
CCACGGAGGGCGAGCCAAAGCAGGGTGGGGCATCACCTCACCAGGGAAGTGCAAGGGGCCAGGGAACGCCCTCCCTAGCCAAGGGAAGCTGTGA
GGGATTGTGCCATGAGGAATGGTACACTCTGGCCCAGATACTACACTTTTCCCACAGTCTTTGCAACTCGCAGACCAGGAGATTCCCTCAGGTG
CCTATGCCACCAGGGCCCTGGGTTTCAAGCACAAAACTGGGCAGCCATTTGGGCAGACACTGAGCTAGCTGCAGGAGTTTTTTTTTTCATACCC
CAGTGGCACATGGAATGCCAGTGAGACAGAACCATTCACTCCCCTGGAAAGGCGTCTAAAGCCAGGGAGCCAAGTGGTCTAGCTCAGCGTATCC
CACCCCCACAGAGCCCAGCAAGCTAAGATCCACTGGCTTGAAATTCTCGCTGCCAGCCCAGCAGTCTGAAGTCAACCTGGCACACTCAAGCTTG
GTGCGGGGAGGAGCATCTGCCATCACTGAGACTTGAGTAGGCCGTTTTCCCCTCACAGTGTAAATGAAGCCGCTAGGAAGTTCGAACTGGGCAG
AACCCACTGCAGCTCCACAAAGCCTCTGTAGCCAGACTGCCTCTCTAGATTCCTCCTCTCTGGGCAGGGCATCTCTGAAAGAAAGGCAGCAGCC
CCAGTCAGGGGCTTATAGATAAAACTCCCATCTCCCTGGAACAGAGCACCTGGGGGAAGGGGTGGCTGTGGGCCTCCTCCTGGCTCTGAAGAGA
GCAGTGGATCTCTAGCACAGTGTTTGAGCTCTGCTAAGGGACAGACTGCCTCCTCAAGTGGGTCCCTGACCCCTATGCCTCTTGACTGGGCGAC
ACCTTCCCGCAGGGGTCGACAAACACCTCATACAGAAGAGCTATGGCTGGCATCTGGCAGGTGCCCCTCTGGGACAAAGCTTCCAGAGAAAGGA
ACAGCCAGCAATCTTTGTTGTTCTGCAGCCTCTGCTGGTGATACGCAGGCAAACAGGGTCTGGAGCGGACCTCTAGCAAACTCCAGCAGACCTG
CAGCAGAGGGGCCTGACTGTTGGAAGGAAAACTAACAAAGAGAAAGGAATAACATCAACATGAACAAAAAGGATGTCCACACCAAAACCCCATC
CAACGGTCACCAGCATCAAAGACCAAAGGTAGATAAATCCATGAAGATTAGGCAAACCAGCACAAAAAGGCTGAAAATTCCAAAAACCAGAATG
CCTCTTCTCCTCCAAAAGATCACAACTCCTCACCAGCAAGGGAACAAAACTGGATAGAGAATGAGTTTGACGAATTGACAGAAGTAGGTTTCAG
AAGGTGGGAAATAACAAACTACTCCAAGATAAAGGGGCATGTTCTAACCCAATGCAAGGAAGCTAAGAACCTTGAAAAAAGGTTAGATGAATTG
CTAACTAGAATAAACAGTGTAGAGAAGAATGTAAATGACCTGATGGAGCTGAAAAACACAACACGAGAACTTCGCGAAGCATACACAAGTATCA
ATAGCTGAATCAATCAAGTGGAAGAAAGGATATCAGAGACTGAAGATCAACTTAATGAAATAAAGCATGAAGACAAGATTAGAGAAAAAAGAAT
GAAAAGGAATGAACAAAGCCTCCAAGAAATATGGGACTATGTGAAAAGACCAAACCTACATTTGATTGGTGTACCTGAAAGTGACGGGGAGAAT
GGAACCAACTTGGAAAACACTCTTCAGGATATTATCCAGGAGAACTTCCCCAACCTAGCAAGACAGGCCAACATTCACATTCAGGAAATATAGA
AAATGCCACAAAGATACTCCCTGAGAAGAGCAACCCCAAGACACATAATCATCAGATTCACCAAGGTTGAAATGAAGGAAAAAATGTTAAGGGC
AGCCAGAGAGAAAGGTCGGGTTACCCTCAAAGGGAAGCCCATCAGACTAACAGTGGATCTCTCTGCAGAAACTCTACAAGCCAGAAGAGAGTGG
GGCCCAATATTCAACATTCTTAAAGAAAAGAATTTTCAACGCAGAATCTCATATCCACACAAACTAAGCTTCATAAGTGAAGGAGAAATAAAAT
CCTTTACAGACAAGCAAATGCTGAGAGATTTTGTCACCACCACCCCTGACTTACAAGAGCTCCTGGAAAGGAAAAACCAGTACCAGCCACTGCA
AAAATATACCAAATTGTAAAGACCATCGACACTATGAAGAAACTGCATAAACTAATGCGCAAAATAACCAGCTAGCATCATAATGGCAGGATCA
AATTCACAATAACAATATTAACCTTACATGTAAACGGGCTAAATGCCCCAATTAAAAGATACACACTCCCAAACTGGATAAAGAGTCAAGACTC
ATTGGTGTGCTGTATTCAGGAGACCCATCTCACATGCAAAGACACACATAGACTCAAAATAAACGGATGGAGGAATATTTACAAAGCAAATGGA
AAGCATAAAAAAGCAGGGCTTGCAATCCTAGTCTCTGATAAAACACACTTGCAACCAACAAAGATCAAAAAAGACAAAGAAAGACATTACATAA
TGGTAAAGGGATCAATGCAACAAGAAGATCTAAATCGTAAATACATATGCACCAAATACAGGAGCACCCAGATTCATAAAGCAAGTTCTTAGAG
ACCTACAAAGACACTTACATTCCCACACAATAATACTGGGAGACTTTAACACCCCACTGTCAATATTAGACCCATCAACGACACACAAAATGAA
CAAGGGTATTCAGGACTTGAACTCAGCTCTGGACCAAGCAGACCTAATAGACATCTACAGAACTCTCCACCCCAAATCAACAGAATATACATTC
TTCTCAGCACCACATCACACTTATTCTAAAATTGACCACATAATTAGAAGTAAAACACTCCTCACCAAATGCAAAATAATGCAAATCATAACAA
ACGGTCTCTTGGACCACAGTGCAATCAAATTAGAACTCAGGATTAAGAAAGTCACTCAAAACCGCACAACTACATCGAAACTGAACAACTTGCT
CCTGAATGACTACTGGGTAAATAAAAAAATGAAGGCAGAAATAAATAAGTTCTTCGAAACCAATAAGAACAAAGACATAACATACCAGAATGTC
TGGGACACAGCTAAAGCAATGTTAAGAGGAAAATTTATAGCACTAAATGCCCACATCAGAAAGGAGGAAAGATCTAATATTGACACCATAACAT
CACAATTAAAAGAACTAGAAAAGCAAGAGTAAACAAATTAAAAAGCTAGCTGAAGACAAGTCACAACAAAGATCAGAGGAGAACTGAAGGAGAT
ACAGACACACACAAAAAAAACCTTCAAAAAATCAATGAATCCAGGAGGTAGTTTTTTGAAAAGATTAACAAAATAGATAGCCCACTAGCCAGAC
TAATAAAGAAGAAAAAGAGAAGAATCAAACAGACACAATAAAAATGATAAAGGGGATATCACCACTGATACCACAGAAATACAAACTACCATCA
GAGAATACTATAAATACTTCTATCCAAACAAACTAGAAGAAATGGATAAATTCCTGGACACATACACCCTCCCAAGACTAAACCAGGAAGAAGT
CAAATCCCTGAATAGACCAATAACAAGTTCTGAAATTGAGGCAGTAATTAATAAGCTACCAATCAAAACAAAAGCCCAGGACCAGTTGGATTCA
CAGCCAAATTCTACCAGAGGTACAAAGAGGAGCTGGTACCATTCCTTCTGAAACTATTTCAAACAATAGAAAAAGAGGGAATCCTCCCTATCTC
ATTTTATAAGGCCAGTGTCATCCTGATACCAAAGCCTGGCAGAGACACAACAAAAAAAGAAAATTTCAGGCCAATATCCCTGATGAACATCGAT
ATGAAAATCCTCAATAAAATACTGGCAAACTGAATCCAGCAGCACATCAAAAAGCTTATCCACCATGATCAAGTCAGCTTCATCCTAGGGATGC
AAGGCTGGTTCAACATATGCAAGTCAATAAATGTAATCCATCACATAAACACAACCAATGACAAAAACCACACGATTATCTCAACAGATGCAGA
AAAGGCCTTCAATAAAATTGAACACCCCTTCATGCTAAAAACTCTCAATAAACTAGGTATTGATGGAATGTATCTCAAAATAATAAGAGCAATT
TATGACAAACCCACAGCCAATATCATACTGAATGGGCAAAAGCTGGAAGCATTCCCTTTGAAAATCAGCACAAGACAAGGAGGCCCTCTCTCAC
CACTCCTATTCAACATAGTATTGGAAGCTCTGGCCAGGGCAATCATGCAAGAGAAAGAAATAAAGCGTATTCAAATAGGAAGAGAGGAAGTTGA
ATTGTCTCTGTTTGCAGATCACATAATTGTATATTTAGAAAACCCCATCATCTCAGTCCAAAATCTCCTTAAGCTAATAAGCAACTTCAGCAAA
GTCTCAGCATACAAAATCAATGTTCTAAAATCACAAGCATTCCTATACACAAATAATAGACAGAGAGCCAAATCATGAGTAAACTCCCATTCAC
AATTGCTACTAAGAGAATAAAATACCTAGGAATACAACTTACAAGGGATATGAAGGACCTCTTCAAGGAGAACTACAAACCACTGCTCAAGGAA
ATAAGACAGGACACAAACAAATGGAAAAACATTCCATGCTCATGGATAGGAAGAATCAATATTGTGAAAATGGCCATACTGCCCAAAGTAATTT
ATAGATTCAATGCTATCCCCATCAAGCTAACATTGACTTTCTTCACAGAATTAGAAAAACCTGCTATAAATTTCATATGGAAGCAAAAAAGAGC
CTTTATAGCCAAGACAATCCTAAGCCAAAAGAACAAAGCTGGAGGCATCACACTACCTGACTTCAAACTATACTACAAGGCTACAGTAAAAAAA
AAACAGCATGGTACCAGTACCAAAACAGAGATATAGACCAATGGAACAGAACACAGCCCTCAGAAATAATGCTACACATCTACAACCATCTGAT
CTTTGAGAAACCTGAGAAAAACAAACAATGGGCAAAGGACTCCCTATTTAATAAATGGTGTTGGGAAAACTGGCTAGCCATATGAAGAAAACTG
AAACTGGACCCCTTTCTTATACCTTGTACAAAAATCAACTCGAGATGGATTAAAGGCTTAAACATAAGACCTAAAATCATAAACACCCTAGAGG
AAAACCTAGGCAATAGCATTCAGGACATAGACATGGGCAAAGACTTCATGACTAAAACACCAAAAGCAATCGCAACAAAAGCCAAAATTGACAA
ATGGGATCTAACTAAACTAAAGAGCTTCTGCACAGCAAAAGAAAATATCATCAGAGTGAACAGGCAACCTACAGAATGGGAGAAAATGTTTGCA
ATCTACCCATCTGACAAAGGGCTAATATCCAGAATCTACAAAGAACTTAAACAAATTTACAAGAAAAAACAAACAACCCCATCAAAAAGTGGGC
GAAGGATATGAACAGACACTTCTCAAAAGACATTTATGTGGCCAACAAACATATGAAAAAAAGCTCATCATCACTAGTCATTAGAGAAATGCAA
ATCAAAACTACAATGAGATACCATCTCATGCCAGTTAGAATGGCAATCATTAAAAAGTCAGGAAACAACAGATGCTGGAGAGAATGTGGAGAAA
TGGGAACACTTTTACACTGTTGGTGGGAGGGTAAATTAGTTCAACCATTGTGGAAGACAGTGTGGCAACTCCTCAAGGATCTAGAACCAGAAAT
ACCATTTGGCGCAGCAATCCCATTACTGGGTATATACCCAACGGGATTATAAATCATTCTACTATAAAGACACATGCACATGTATGTTTACTGC
AGCACTGTTCACAATAGTAAAGACTTGGAACCAACCCGAATGCCCATCAATGATAGACTGGATAAAGAAAACGTGGCACATATACACCACGGAA
TACTATGCAGCCATAAAAAAGGATGAGTTCATCTCCTTTGCAGGGATATGGATGAAGCTGGAAACCATCATTCTCAGCCAACTAACACAGGAAT
GGAAAACCAAACACCGCATGTTCTCACTCATAAGTGGGAGTTGAACGATGAGAACACATGGATACAGGGAGGGGAACATCACACACTGGGGCCT
GTCTGGGGGTGGGAGCTAGGGGAGGGATAGCATTAGGAGAAATACCTAATGTAGATGACGGGTTGATGGGTGCAGCAAACCACCGTGGCACTTG
TATACCTATATTACAAACCTGCACGTTCTGCACATGTATCCCAGAACTTAAAGTATAATGATTTTTTTAAAAGCACGAATATTATCCTCATTAG
CTACCTTATAAGAATGTTATATTAAATGATGTATTTTGTGAAAAAAAAAAAAAGTTTGTTCTTGAGGTCAGAGGTGCCTCATATAACACCAGGC
AGACTAATCACATGGCCGATGATTAAAAGAGAGATAGACAAACTTCTCATTTTTTAGATCTTTGGAAAAGACTTATTGCTCAGGTAGGAGTAGC
CAGAAAACCTCAAAAAAGAGCTCGAGGGGGCCCAGAAGCAGAATCAGATGCAAGTGAAGTGCAGAGTACTCAGAAAGTCCAAGAGAAAAACACT
CGCTGGGCATATGTAGGCCTCTTTCTTTAGAAAACTCAGCAAGACTCATGTATTGAATGAATTAATAACCTTGGTTTCAACCATTTGTATATAT
GTGGACTGTAGGTGTGCAGATGTGTGTAACTGCCTACATGTGCACTTAGTATTATGCATATATGTATATTTACATATAAATATAATGATGCATT
TACATAAAGGCATCCATTTGGCATACATACCTATGCATATGCAGTATAAGGCTGGATTATTCCCCTGCCACACACAAAGATTTTGCTGTCCAAA
GACTCAGTGCAATGAATGGCATGGAAGGGGCTCTATTTTCAAGGGTGAAGCAATAGAGAAATACCGAAGGCCTCGGATAACCTGTGATGGGGAT
CCCACGAAAGCCACCGGGGACAGTGCCAGTAAGCTTATCACTCCTGCCATAGACAGCGGACTTCAAAGAAAAACTGAAACCCTTTCTCCGGAAA
TAATCAGGAAGGATCTTATTTTCTTCTTTGAGGTCAGCACATTTTGCTTTTCAAAATCAAATCCACAGCAGTTCATTAGTAATGTCCCACAGGT
ACTGCCTCAAGTTGGCAGCTGTGGAAGCTAAAGAAGCATAAGACCTGATTCTTGACCTCGGACAATTTACAGTTTAGCTGTGGAGGTAAGACAG
GGACACAGAATATTATGTGGCAATTGAAAGTGTAAATATTATAGATTTAAGTAGGGTAAAGAGCTCATTCTGTAGTAGAGGGGTCAGTGAAAAG
TAATTGTTCACCTGGTAATAAGAGAAGATCTCTGTGAACAAAGTCTAAGACTGCAAACTTAGGACTGTTGGGAGATAGTCAGATAGATTCATAT
ATAGATACAAATCTTTACCATCCTCAGACATCCGCCAACTTGGTTGTTTAGGTTGTAGGGCTTACATGAGGAAGGGTGTATAATAGATAACTCT
AAAAATGTTTGAGCCTTAGACCCAGTGAGTGAGAAAGAGTGTCATCATTTAATATTCTCCTGACATTCACCTGCCCCTTCTGCATCTGTGGCTG
TGACTGAGGACTTGGTTCTTTATATCACCCAGTGTTCATGAGGATTCTCTGGAGAAATTTCTTGCTGGTCTCAGTATCAAGAAAGGGGGATCTT
CTCTTGAGCAATTTTGTTTTTGGTCTTGCATGTGATAGGAATTGAACTTTAGACAAGGCCTCTCTTTAAACTTGACTGCTAGAAATGCAGGAGA
CAAATTTTGGTCCCACCTGTAGCAAGGGTATTTGACTCTATGAGATGCATTTATTTCAAATGATGTTCATTTTTGGCTCCATCTTGTGTGCCTC
ATTTTTCATTTCACACACTTCTTTTTGTAGGTTATGTGCTCTTGTAAGCTATCTCGAATTCTGTTTTAAGGACAAGGTAGCATATAAATATAAG
GAAATATATAAATGAAACAGAGGAAGGCTTTAAAGTCCAGAACACTATGGCCAGGAAGACAACTATGACTGTGGCTAGACTTATGGTCTGTGGC
CGTGATAATGAGTGTGTTATTAGGGCAGTCCAGCAGTGATTACAGGGAGTCAGACAGACCTCAGTCCAAGTCAGAGGTCTCTCTGCTGTTTATT
AGTTGTATGATCTAAGGCATAGCATTCTCTAAACCTTCATTTTCTCATCTTTACAACAGTGATCTCTTTGTATCAGGGAGAGTGGATTAAATGA
GGTAATGTATACAAACCACTTAGCACAACCGCACTCAATCATTAGCTATTGAGATTATCAGCACTATCACCTGGACAAGCAGCGGATCGGGCGC
CCCTCTTGGCCAGGTCTTCTGTGCGCACTGACGTTGTATGAAAGTTGTCCCCACATCTCCTACACCCACCGACGCCAGGCGGCCGCGTCTGGGC
CAGATATGGATCACGCCTGTGGAGCGCACCTCCACACAGGCAGCCCTCGTCCAAGGTGCAGGGCGCAGGGCACAGGGCACATCTCTACGGCCCA
GTCCTGCAGACGGCTACCTAGAGAGGGCAGGCCACAGGTTAAGCCAACTTCACAACTGTCTTTCCGTTCCTTTCCCCTGCCGGACTCCGCGCTC
TGATATATACAGGAGAGGCCCTGGAGGGGAGAAGGCGTCCTCACTGCAGTGCTCCCAGCAGCCGCCGCAGGAAGAGGCGTGGAAGCTGCGGGTC
GGCTGCGGGAAAGGGGGTCCCATGGGAGCCGCTAGCGCAGCCCGTTTCCCGAGCTGAGGTGGAGGTCGAAACGCACAGGTGCCCTGCAAGGCGC
GGGCGTGGTAGCCGGGAGCCCGCTCCTCTCCAACTCCCCTGAGACTTAACCCTTTGGCCAAGAGAGGTCGAAGGCGGGCAGGGGTGAGAGAACC
GCGAGGCGCAGGCCGGGCACCCGCGGAAAGGGGGCAGGTTTCGGGGAAGAGCGGGGCGCCGCACGTTTCCTGCAGCCGCCAGCATCAGCGTGTC
AGGTGGTTGCCAGGCGGGTGTGAGCGCGCGCGCGAGGCAGAGGGGGCGCGCTCGCACAAAGTTTGTGCCTGCGGTGCACCTCGGTCGGGCTCGC
CGGGCTCGAGGGCTGGCGGGCGCCGGGGCTACGAGGCCCGGGGGGGAGAGAGCCGGCAGGCGGCGGCGGTGGTGGCGGGGCGATGCGCGCGCC
CGGCCGCGCTAGGTGAGCCGGCACCGGGAGCCGGCCGCGGCCATGGGCACCCTGGGCAAGGCGAGAGAGCTCCGCGGTGAGTAGAGGCGCAGTG
GCCCTCGGGAGCGCCTGTGTCCTTTCTTGGGAAAGAGGCCCCTGCCCGTCGCCCCAACACCCCGAGGGAAGTTGAGGGCGGAGGGGGGCCGGGG
TCGGCCGGAGTCCCGGGACACCAGCGCGACGGGCGGACGTGTCCAGAACGCCCGCGCGCCCGGGGTTTCGGNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNCGCGGCTGACGTGGGGCCATTGTTCATCATGTACT
CCACTCTCACTTCCTTCCCAACCTGAATAAACTGCAAAGTTCCTTACACCATTTCAGCAGTCAGATTTACTCGGCTTTAGCAGGCCACCATGAC
TATTTCCTGAATAAGCATTTTTCTTCTTTCTCTTTCACTATTGAAAAAACAGGGACGAGAAACATGTTTATCACACCACCTTCCTTCCTTCCAT
ACCTGTTGTGCAACTGCCGGCCGAGTTCAAGACTGTTTTTCTAATCTTCAAAGAATCATGTTATCAACCTGAAAAATACATGACTGATTCTGAA
GTCCTTCCCGCCAAATAACCAACCCTAATTCAATCCCCTTAACAGACCTTCCAAAGGCAACGACAATATATTGTGTTCTGGAATATGTTGAACT
GAAAACAATGGCTTAAAATCCCAAAGCTGTAAAAGTCCCGTTGAGTCTAATAGTCTTTTATCCATTATCAAGCAGCATTGGTTAAGCCCTTAAT
TGTATCTGACACTGTGCTAGGCAGTGCTCAAGAAGAATTACAGCGACCAAACTCTCCCTTCCATTAGAGCTTACAACACCTGAGAAACATACTG
TTCAGGAAATATATATATATATATACACACACACACACACACACACACACACATATATATACAAAGGACAGGGTGATAAATACCGAAGGCATGC
AGGAAGAGTAAGTTTTTATACCATGTAGAGTTTGAGTAAGTGCACCTCATGGGTGAGGACATACGTCAAGGAGAACAGCCACCATTTGTTCAAG
TCCCTCACGGGCCAAGGGAATGCTGCGGGAAGTCAGTAAGGTAAGATCTCAGGGTTTTGTGTATTATGTACTGCAGAGTTTCTCATCACAGTAC
ATGAAAGAGGCAAAAAAGAAAAGACGCTCTGTGTCTGGAAGGAACAGAAGCCAGCACCTTTTGTCTGTCTCTCTTCTTGCTCTTTGGTTATCAG
TTGACCCATCTATGGGGCTGGGACATGGAAATTGGGCTACAGAGGAAATGGAGCCAGGAATCAGCAGCAGACTTTATGAGGGACAAAAGCCCAT
AGCAAAATATATGCCTGTGCTTCCGTAGGTCTCTTTAAGAGGTCATCTGACAGTAGAACAGACTGAGGCTAATGCTTCTCCTGAAGTTTACCTT
ACTGGTTAGTTTCTCAGGTTTGTAGAGAAAAGTGGTGCTTTAGATTAAAGCAGTCTACCTAGCTTTTTGTTAATGAGTTTAGATGTCTTCTCAT
TTTAAACCCACGAAAAACTGTACAGAAGTGTGCAGGGGAAATTATTATCATTATTATTATTGGGACTCTTTTGCCTCAGACCGTAATTTCAGCT
ACTCCACCCAGCAACTTGAATAAAAGAGGGAAAGAAACTGTTTTAAAGAGAAAGACTGAATAATTGTTAATTTAGTTTCTACTGCCTCTCAACC
TCACCTTCTTGCTTTCAGGAGGTTTGAATTGGCTACAGCTGTCCTGTCCAATGCACTGGCCACATGTGGCTATTTTCGTATACATTTCAATTAA
AGTTAAATGTAAGATTTCATTTCTAAGCCTCACCAACCATAGGCGAAGTGTGCAATAGCCACAAATGGCACCTACTGTTGGACAGAGGAGAGAG
CACTTCCATTGTTGCAAAAAAGTCAGTTGGACAATGCTGGAGAGGGTAGATGTTTGTCGCAAAGGAAAAAAGAATAGATTTTCTACAAACTGTG
GGATTTGCATTTTCCTAGCATTTCTAGATAAGAGTCTGAGTATTGCTTAAAGCATCGTGGGCATTCACAAGTCTCACTGATGGCTATCTCTTTA
GCATTATTAACCATTAATTACCACCCACACTGTTCTCAGAAGAAGGACTTATATTGCTTTTAAGGGCAGTGGCTAGTACATATAAATTAAGAGC
ACTTTCAAAGGCCTCTAGCAACCCTCTCAGTGTCACATACAAGGTTACAGATCTGTGACTAGAAACTTACGGCCTTGAGTCATAGTTCATTGCT
TTAACCATGAAACAACCCACTCCCGCCTTCTGTTCTAACCAGAGATCATGCTTTACATTCTCAGCATATTATTGCTAGTTAATCCTCTCTTCAG
CTGAGCATAATAAGGCAAATCATCACAACCTGGTTACACTTATGTCAAATATATGTGAATCACACCATCTGTTTCAGAGGTAGAAAAGACCTAT
GAGATCATCTGGGCTTTTATTTTTTTTCCCTTTACAAGAGGGCTGCCAGCATTTGGGGCCAAATGGAAGGCTTCAAATGTCTGAATTCACAAAG
CTCTTTCCTTATCTCCCTCTCAATGCAAGACAACTTCTGACAGCATCGCAAAGAAAACTGCACACCTGCCTCAGAACGGGCCCCCCCAACACAC
ATACACAACTCCACCCCAGCAAGCAGACCTCCTACTCCCAGTGAGACGTGGGAAGAAACTCCATGTTTCATCTTACCTATTAGTGTGAAGGGAA
ATGCTTCCTAATATCTTCTATTPATGGACTGACTTATTTTTTCTTAGAGTTCCATTTGTATTGATTTTCTTTTATACTGAGAACAGCTCTGAAA
TAGGCAGGTCAGATATTACTCTCTCCATTGAACAAACAGGAAAATAAACCTTTGAGAACTGAAAGAATTTGCCTTGTGCCCCCAGTCTCAAGAG
AGGATATGCTGCTCCTGTTCTCCTTCCCCTACCCCTAGCAGCCACTTGCAACCTGGGTTGGGGCAAGCCTGGATCTCTCTCCTAAGACAGGCCT
TCCTACACTCAGGGGAGACCTGCATGCCACATAGGACAGGGCTGCACCTGGTGCCCACTCTTCTGCAATCCTGGTGTCTCCCCAGTTAGAAATG
AAACTCCATTCAATGTAAGAAATATTGATTGCACCCAATCTATATACCAGGTGTCTGCCTGATATTCTCTATTGAGCAAAGTGTGAATCACTGA
ATTCTTGTGTGAATTGTACTAATGCTATCCAAAGACCTTCCAAACTAGAAATACCCCAAAACTAATAAGTATGTTTTCTCCACCATGGGAGCAG
TTGGATATTAAAAGCATTTTATAGGCCTCAGCTTATTGAAAGTAAAACATTCCGTCTCTTTTCAAAATGGGAGCCGCAAAAGGCAAACACACAC
TCAGGGCCATAGCCCTCTCCGTAACAAAGGAAAGATTAGGTATTTCTTACCACTCTGCAACTTGGTCTGTGCACCAAAGCAAGGTCCCCAGAGG
GCAACAGGAGCATAAGACCAGAAGCCAAGGGCCATGACATCATTGGGGATGCAGACCTAATAACAGGCAACACCAAGGAACAAAGTCAGTTGTA
GTGTCAGGAAGGGGCCGAAGCCTTAGCCACTGTTGATGGCAGGCCCTGTGTGAAGAACACAGCTTTCCCAGAAGCCTCCTCAGTGGAACCTCCC
AGTCGCTAGTAGTCGTGGCTACAAGATTTCACGTGTAAAGGATGGTCCTCGGGGGCACTTGCATCCTGAGGCTTCTAGTCATCAGAGCAGCTCC
CAGGCCGCTCTTAGCCCTCTAGAATGAATTAGCATTATTATAAGACTGGACACTATTTATTCCAAGGAGGCCTTTGCTGCATCTTTGATGAGCA
GGCATCACCAGTTCTCCACGCTGGTCTGCCTTCTCATGAGCATGGGTTTCAGTGACTGCCAGGCCTGAGAAAGGAGGAGATGGGAAATCCCCTT
CATTCATATCTGTGCTCTCCTGCTTGGCAGTGTTTGTTGCAGGCCCCGCTGTAGAGAACAGAGGCAATGCTTATGCGTAAGTTAGACAGTGTCT
CAGGAATTCTGAGCAGTTCAATTCAGTAAAGCCATCCAGAGAGATGAGTTTTACTTCTGAGAGAGCTGGAGACAGACAGTAGTTTAACACTTAT
TGTGGACTCTGTAGGATCCTGAGCCTTTACCTAACTTGACCCAATTAATCCTCCCAACAACCCTATGAGGTGGTTGTTATTTTGCTCCTTTAAC
TAATAGAGAATTGTAGCTCAGGGAAGTTAAATGACCTGTGGAAAGTGGTTCAGCTCATTAGTGAGAGAGCTGCTGAGATGTGAACTCTGGTCAG
TCTTCCCCCGAGGTTCGCGTTCTTTCTGCTACTCCAGAGATTCTCCAACTTTGGCGTGAGGAATAATCAGGGAGTGTGTTGAACATTCAAATTC
CTCACAGTCAACCCCAGAGATTGCCATTCAGCCAGCAGTCACTGGTTTGGATGGAGGTGACCCATGAATGGATGTTGAGAAATACTGTCGTATA
GCACACTTGCTTCCTGAACATCCCAGATCATTTTTCTCAGACATCCTAGGAAAACCTGAAGGGCACAAATCTCCAGGGAAGCAATAGAGCATAT
AGGTTTTTCAGCTATGTGAACTAGAAAAGCACATGGTGAAGCCAGCCTCTTGGAGATCTCTAAAACAATCCAGCCAGATCTAATGAGATCCCTG
CCTAAGAGCAGGTCTGAAACCCTGGGAGCACTTTTATCAGATTTTTCTGCATTCCTATCTCTTCTCAACTGCAGTAAGCCCCCTATAAAATGTA
CAGCACGCCTGTCTGTTGGCTGTGTAAATAGAATGTGGATGTCTGTTACAACTACAGTTTGATTCTTGCATTCAAAGGCCATATTCCTAGTGTA
CATTTCCACCTTTATTCAACACTCCAGTTTCCTATGGTGCTGGGAGGGTCTACTCCGTAACTTATGCCCGTCCCCATGGGTCAGAGGACTACAG
TAGTGGCTTAGCTGGGGAGGAACTGGGGAGTGCTTCTCCTGAGTACTTACCCAAAAGTACTACTTTGGCAACAGATTATGTAAGGCAATATGTT
GAACTGAAGTTTATGAAATCTGTGATGAAAACACATTGGGAAACTGTGTGAAAGCAAAAACAGTCTTTTCAGTAATTGACAGAAACAGCTGTGT
TGGACCCTGAGACCCCTGGAGAAGGTCATCTACAGTTCGGGCCCCAGCCGCAGCCCACCTGCTGATGTTAGAACTTGTCCACTCCTGCCCGGAA
CAATTATAGCTGCTGTGTCTTGTTCTGGAGTTACAGTGAGCTTTGGCTACAACCTAGTTGGGCTTGCCCTAGAGTGGCTGGAAGGAAAAGAACG
GGGAAAGAGTGAGAATGTAAGCAGAGCCAGCTTCCTGGGCATGCCACCTCTGAAGGCCCACAGGTTCCCATGCTTGGTTTAATGCTGTCACAGT
CTTGAAATTTTTAATAATTTTTGAATAAGGGACCATCATTTTTAATTGCACTGAGTCCCACCAACTATGTAGTCAGAGGTCTGTGTAAGAGCCA
CAAGACCAGAGGGCAAAAATAATTTGCCATATATTGAAGACACATAGAACGATGTGGATTTATGTCTTAATTTCTTTCTTCATAGATGCAACTG
TGAAACAAAAATTATTTCATTATCTCCTAGCAGTGGTGTGCTGGGCTGGCTCATACCAGCTCACAAGAGCAGATTGTTAAATTTTCAGGAATTT
TGCAAGCTAGTTATTAAACACAGCCATGATTGAAAATTAAGTTATATATGCTTACAATTAAATTATATTAAAAACAAAGATAAGAAATACTCAA
AACTCACCACTTCCTAATTCATGACTACATTTTACTATTATCTTTGCTTTTGAGGTTATGTCTGTTATGTCTACCTGGTACAAACGCTATATAA
TGGTATGCTAGCATGCATCTTTTCCCAGCTCCGTGTTCAGTCAAGTCATGTTAGTAGCTTGTTAGTAGTCTGTTGGGAGTATTTACACCGTGGA
AATCAACAAACATCACTAATCAGGGCTTCCTTTTCAATCCCTCCCCTGGAGAGCCCATTGTTAAACATTTACCAGCACACCACTGTTTCCCAGA
TACAGAGGGTGCTTACATAACAAAATGTTGAGGTGGGAAGGGACCCTAAAAGCTGAGACTTCTATTTTTTATCTCTTAATTAAGAGAAAGTAAT
AATTCTGCTATTATTTTGCTTTCATCTATGTATCTTATAAAACAACCTCTCTGTGTGTTCTTCAAAGGAAAGGAACAATGGACCTTAGAGATAA
GGAAATCATGTATCAGTGTGGTTGAGAAGTTTTCTTAAAGAAGTGACAATGGAACATGAAAACAGCATGAGAGATTCTCATTCTGAAGTCAAAG
TTATTTCGTGTTTGTATTGTTTTAATTCACCTCTAAAATATCTCTTAAGATTAACCAGCCTCGTTTCTGACCAACCGTGAATTTTGTGAACTTT
TGTTCTTCATGTATTATATGCTATCTCTATTTTGATTTCTGATTCCATCTGCTTAATCATTTGCCTTCATTACCCACTGCATGGTAACAGGCAC
TACTGGTGTCTATAACCAAGCTTTTGTTGAGTATGTTTTCATAAGTTTGTTTGCCTACTGTATTTCCAGTTTTATGAACGGTCTGTTCATATCC
TTTGCCTATTTATCACTTGCTGCTCAATGCTTTTATTATTAGAGGCGTGCAAAAGCTTTTTATATAAAAAGCTGTTAACTCTTTGAATATCATT
TACTATAGGCATTTTTTTTCCTGTTCTCATTTTCATTTTTATGTTGGTTTTGATATTGTCATGTATAGAAACTTCGGATTTTTGTGTTTAAATT
TGCCCATTTCTATTTAGCTTTTTAAAATTCTAAACCTTAAAAAAATCCCTATTCCAAAGATTTAATTAATAGTCCATTGTCATTTTAATCAGTT
TTACTTAACCCAGTGTATAATTCCATATCATACATTGTCGTGGATGATCAATACAAGCTTGTCAATAAGTTATTGCTGGCAAAAAAGAAGCCTT
CAGTTTTGTTTCTGAGATTGTCATTTCCTTAAAACCACAGCGCTTGGACTGGTCGGTTTGTAACTCAGAGCCTCCGTTTCTCGCTGTGATACCA
TTAAAATGATTTCTGAGACTTAAAGTGTCTGGAAGAAGGTGTAATGGGGTATCTCCATCACGATATTAAACTTGAAGAAAGTCCTTCAGGAATT
TGCATATTTTAAGAATGTCCTTTTCGATATATTTGAAGACTCAATATTCTTAGGATGGAGTCTTAGAAACCCAACAAATTTTTAACTCAGTCTT
TAAAAATTATTAATTTAGACTGTTTCTTGTTCTTACATAGACTTTGTGGTAAGATATACATAAGATCAACCACTAGCATAGACTTTTAAAATAT
TATTTTAGTAATAGCTAACTTTTGAGCATTTACTATCAATAACACAGTGTTTATCTTAATATTTTACATATGTAGTCTCATTTAGTTCCCACTA
TTTTATAAGTTCTTGTATTTCCAAAGATCTAACTGTCCCTCACCATTTATACTTACTTTATAAATGTTAATAACTTTTAAAATTATTCATGTTC
ACCATAGAGAACGTAGAAAATACAGACTGGCAAAAAGAAAAAAATTACCGTCACTCATTTTACCATCAAGACCAAGATAAGCACTATCAACTTT
CATGGGTGTGCCTTTCTTGATTCTCTTCTATGGATATTTACATGTATTATAATCACAGTAACTTTTTGGTGATAAACTCACTATAACTTTTTGC
TTCCCATAATTTATATCTTAAACAATTTTGATCTTGAAATCAATATTATTATTTATAAAGAGGTATGCTAGCACACAAGGTTGCTCTTCTATTT
TAAAATTGATTTTTTAAAAATAATTCTGCTTAGTCCCTTCAAATATAGGACATAAAAGTGAAGGTTGCTTGGCAACTGAAATGATGGTAATGAG
GAATTAAAGACATTACAAGAAAAGAAGAGTACTCAGTTCTTTTCAATGCTACATGCTAATTGCAGTAACTATGGCTGATTACAGTTGTAATAAA
TGAAGGAAACACAAAACTTCCATTAAAAATTCTTGATGTGAACTTTTTTTTATTTTGTATTACAGTGAAAGTGAGATGGGGTTTGCTGAAGAGA
AACTTTACTTTCAGAAACAAGAAAAGATCTTTTGTCTGTATTAACTTTTTTTTCTTTTCTTTTCTTTTTTTTTTTTTTTTTGGCGACAGAGTCT
CACCCAGGCTGCAGTATGGTGGCATGATCTGGGCTCACTGCAACCTCCACCTCCTGGGTTCAAGTGATTCTCGTGCCTCAGCCACCCGAGTAGC
TGGGTGTGCACCACCAGAGCCAGCTAATTTTTCTATTTTTAGTAGAGATGGGGTTTCACCATGTTGCCCAGGCTGGCCTTGAACTCCTGAGCTC
AAGAGATCCACCTGCCTCGTCCTCTCAAAGTGCTGGGATTACAGGCATGAGCCACTGTGCCCAGCCTATTAACCTTCTAAGGGAATATTAAGTT
AGCAACTGGGTGAGATACGTAGCAGAAGCACAAAGATTTGGTACATCTGTGATTCTTCAGCAGCACAGAAGAGATGGTGGGCAGTGGTGTGAAG
GAAGCCATGTTAACTACTTGTTTTTTTTTCCCCAAAGAGTAGTCAAGGGCCCTGGAGAGATTGCTGAGGTTCTCACTAGAGCCTTCAAGCAGAA
GATGCTTCCTAGGATGGAGATGTACTTACAAGGACACCAGTGATTCATTGAAATGGACAAAGGGAGTAGAGACTAGAGGGGGAAGAGGGAAGCA
GAGGGAAAGAGGGAAGCAGAGGGGAAGAGGGAAGCAGGGATTGCCGGTTTTGTAACAAACAATGCAGTCACCAGAAAAGTAAGGCAGTTTGGGA
TGAAGCCATCCAAGAACTTATCAAAGGTATATCTTTGCCACAAAGTGAGGATTTCTAGTTGGAGGAGCAAATGCAGCTATGTCTGTCTCCTCTT
CACTGATCTATCTGAATCATGGTATAGGACTTGGTTTTGTAATCTTGTGAACAGCCTCATTTTAAGGGGGAACCCACAATATTCAGGAAGCCAA
GAAATGTCCCTGTTAATCATCTATGAGTCTATCTTGCTGGAATGTAGTTATAGACTATGGAACATCCTTAAATCTGATGAGCAATTAATCACTG
GTTAATTTTGGATTCACTTATTAAATGCCAAATGCATAGGGGATTTTTCACACTCTCTGCCCTTCCTCCTTCCCAGTTCATGAGCACCACAATT
GGATTCCATATTTACTTCATCCAAACCTACCTTGGTGGGTTCACCATTTACCTCCAGTCCTGGGGAGTTCAGTCCAGACTGTGAATTACTGCCA
TTTCTGAGGGGCTGCTTCCCAGATGGGAGCCATAGGACCCTCGCCTCTGGCCCTGGACTCAGTCTTCGTTTATTGCCAAGCATGAGCACTTGCT
CTCGGGAATGGATGCTGGCGTCCACCTGACTGTTTCGCTTGCTTTAATCCCTAAAGGAAAAGCTCCCTACATTTATTGTGTCTGTGGTCCCCCC
ACCCTCTCTGTCCCAGTGCCTAAGAGTCTCATTACCTTGTCAGTAACTACCAGTGTCTACCTGAGATGGACTAAGCTTCATTTAACTGAGAAAG
TGCACCCTCAAAATTGCAAGGAGAAATGTTGGAGGAGGACCCTCTGGATTAAAAGAGACTTAAAAGACATTTCAAACAGTCATAGTATGTGAAC
CTTTTTTCTAAAAATTATGTGTGTGTACACATACACACTCATAGGAGTATAGATGGATCCACATCTGCTGTATGCTGATCATTGTTGCAGCTGG
CTGAGTGCATGGAGGTCCCTTACGTTGTTTTGCTCACTATGGTATACATGCTTATAATTTTCCATAATCTTTTTTTGAGACAGGGTCTTGCTGT
GTCACACAGGATGGAACACAGTGATGCCATCTTGGCTCACTGCAGTCTCTCCCTTCCCAGCTCAAGTGATCCTTTCACCTCAGCCTTCTGAGTA
GCTGGGACTATAAGCATGTGCCACCATGTCTGGCTAATTTGTGTGTGTATGCGTATTTTTTTGTAGAGACAGGGTCTCACTGTGTTGCTCAGGC
TGGCCCCAAACTCCTGGGCTCAAGTGATCTGCTCACCTCAGCCTTCCAAAGTATTGGGATTACAGGCATGAGCCACTGCACCCGACCTCTAACA
ATATATTAAAATTAAAAGTGTCCTCAAACCTAGGACAAATAGATGATTTAGGATTACAAAAATTCAAACGGATTTTAGGAAATTTCTCTAAATT
TTCTTAGGTGGCAAAATAGAGTCGCTGTGTCTGTTTTAAAGAAGGGAGAGGAAAATAAAGAATGAAAATGGTTACTTTGGTTACACATCATGAA
AATAATTCACCCGTGTCATGTACTTGACATCTCCTGTCACAGCGCATTCCCCAATCAGCACTGAGATGCTCAATTCCTGGGTATCCAAAAAGAC
CAAAAAAAAAAAAAAAACCTGGAAACTTGTAATCTTTAATGCTACTGTCAGAGGCGGGAGTTGGAGTTGAGTAAGTAAGAGAATGTAATACTCT
CCTCCACACCCCATCTCACACACACACACACGCGCGCGCGCACACACACACAGTTTTGAGCAGGGAAATGTCATGACTGTGCCACAGCTGATTT
CAGCAAGCGGTGAAAAGATTATGGAGGCCACGTCACATGGCAGTCTTTGGAATCTGAAGCTGAGTCACTGAGTCTGCCATTTACCACATGTGGG
GCTTGTTTGCCCGTTTTTAAAATGGAGAATAATGGTAAAAAATTTTAAAGGTGGTTTTACTTTGGGTGGAAATTTAACAAGATTGTCTTAGTTT
GAGTTTTCCCAGGAGCAGATCCTGAGACAAGGATTTTTAAGTACAAATAGTTTACTTTGGAAGGTGGTCCTAGGAAAAACTGGTAGGGGAGTAG
GGAAGGCAGCTCTTGGAGGGTGCATTGCCAAGTTACAACTCTGGGAGTCAGAGTGGAAGGCTCTTTTCAGACCTATCTTATCTGAGGGAAAGGA
ACTGGAGCATTCATGTACCAAGTCCCATCGCTCATTGGTTGAGGGCTGCTTTTAGAGGGTCTTAAGTTTGCAGCACATTTGGCCTGCCCTGCAC
ACAGGTGGGGGTGTCAAGGGCTTCCAGAAACCTCTCAGGCTGGTGGAAGTTGAGTTGGCATGCATTACAATGGGAAGGTCTGAGAGTATGTGTG
TGGGAACTTACAGGCTCTGCTACGAAGTCAAAAAGTAAATGTTAGATGGGCAAATATAGGCTACATGAAAATCCACATAAACAGTTTCAAAGCT
AGTTTATAGTCCTACTCAAAGTGTATACATCTAGATATGAGTATTGAAGGATGTCCTTCTGCAGGTGAACCATTTATGAGTGGCTGTCGACATT
TTCATCAGATTTAAACACAATCTGTGTTTTTGGTTAGACACACTGTGGGAAATAGCATAATTAGCAGAATTCAGGATGTGAATAACATAGCACA
AAGAACTTAGACAAACAGGACTTCAATAAAACGTAAAGTCTCGCACTTAAGTTTAAAAATAGTGCTTCCTATATGTTCTGGATGGGGGGAAGCT
GTATTAATTGTAGTTCTTATGACCAATACCAAGAGATTTTAGTAGATTGTAAATTCAACATGAGTCAGTAGTGTCGTGTGTCTTCCCCAGAATA
TTAACTTTCCAAAAGTTAAAATAATTAGAGATTGCCTCAACAGAAATAGCCTTTCCATGTTAAGGGAGTAATGCCTCCATTCTATCCTGAGGTC
CAGTCCTAATGCTGACACATAGTAACTTTATTCCCTTAGCCGGTGTCTTAATGTTTCGGAGACTCAGTTTCTATCAACCTGAAAGTTAGTAGAT
ACTTCCCCTGGATTGTCGTGAAGTAAGCATCTTACCAGAAAGTTAGAATTTATGGCCCAGCACGGTGGCTCACGCCTGTAATCCCAGCACTTTG
GGAGGCCGAGGTGGCAGGCGAATCGTGAGGTCAAGAGATCGAAACCATCCTGGCCAACATGGTGAAACTCCGTCTCTACTAAAAATACAAAAAT
TAGCTGGGTGTGGTGGTGCACACCTGTAGCCCCAGCCGCAACAGAATACTCTGGAGGCTGACGCAAGAGAATCACTTGAACCCAGGAGGCAGAG
GTTGCAGTGAGCCAAGATTGCGCCACCGCACTCCAGCCTGGTGACAGAGCGGGACTCCATCTCAAAAATAAATAAATAAATAAATAAAGTTATA
ATTTATGTAAAATGTTATTAAAATTCCAATCTGCAGTACATACTCAAAAACAGTAGCAATCATTATTAGTATAGACTTGTTAAGTGTTAGTGTA
TGGCTTTTTCAGGTTAATAATGCAGCTTGCCAGAGGATCCAAAATCACATCATGGAGATACAAGATGGTTATAATGGCTATGCCAAAAAAATTA
CCAGGATGGTGTCATAATAGACACGAACAGCTATGAGACTAGAAGAGAGTGTTATAATTTAAATTTTCCCATGAATTTGAGCTGTATTAATGCA
GTGTTTAGTAAGGAAGACCTGAAAAGTCTCTCATTTTTCAAAATATTAGGACAATGATGGAAATCCAACCAGAACTATCTTTGGGAATTTATTA
GAAATAATATGGAGTATTTCATATATCTAAAACACAGGAAGAGTACAGGATGCCTGCAGGCCTTAGGAGTGCCTGAAATTAGGTACTATAATAC
TTTTAGGAGACTATTGCATATGTTCTGTGTATGTATGTGTCTCTTCTCTGTCTGACTTGCTTCTCTCTGCTTGTCAGCTCATCATTTTCTTTCA
CTGAAGATTTGTTTTCTACATGGCACTAACAGCTCTAAATCATGCATCTCTTACCATTAGAAATTTAATATTTTTTTCCCTCAAGTTCAGTGAA
AAAAAAAAAACTGTCAGGGAAGAACTTTGGTTCAGATTTGGCCAGGTGGCTACTGCTTGAATGAATTGACTGTGGCCACAGGGTTGGAGTCATT
TAATAATCCAGCATTTCTCTCTAAAACCACATGACGGAATCTGGGGAAGGAGCGTTTCCTAAAGGAAGGCTATTGTGCTGGGCATATGAAGCAG
TATCTGTCATAACCAAGATCTGACCAAACACTAGAGTGCAGCATGCTATTAGATTCAAGTTTAGTTTCTGGATTTTAAGATGGGAATCGAAAGG
CTAGAGAGAGTGCAGAGGAGATAAATAGTTAAGAAATTATACAGAAATAACATCTGTGACGATATAAGATGGCAGAACCTGAAAAGAAGACAAA
TAAGTGGGTTTTCCCCTTTGTTTTCTTAATAATGTAAACATGTATGTCTCAATAGTAATAACCATACAACAGCTAGTATGAGTTATTTACCTCC
AGTGGGACATATAAAGGGTAAACAAATTTAAATCTACCAAAGAACTGGGACTTCCCATAAAAAAATTAAACATCAAAAGGAGAACTTTCATATC
TGACCATAGTGGAATACTGGATATTAGAATTATTTTCCTGCTACCAACAGCTATAAAACTGGACAAAATATCTGAAGCAACTATTTTTGGGTCT
CAGAAAACAAGACAGACATGTGATTGTGGATAAAATAAAAAGATGTGAGGTTAACTCAAAACTCCTCTTGGCTTTCTGCCTGAGAGCACTTTAC
AAATCATGGTACAGGAAACTGGAGCCCAGTGAGCAGCATCTTGCCTTGCGGAGTATCTTAGTTCGTTTTCTGCTGCTGCAACAGAATACCAGAC
TGGGTAATTTATAAAGAGATTTATTTGGCTTATGATTCTGGAGGCTGGGAAGTGCAAAATGAAGGGACCACATCTTGTTATGTCATAACATGGC
AGAAGGCATCACACAGACAGAGAGAGTGCTTGCAAGACAGAGAAGAAGGGGGCCAAACTCCCAAGATAACTAACCCTTTCCCAAGATATGGAGT
TAATCCATTCATGAGGGTGAACCCCTCGTGACCCAGTCACCTCTTAAAGGCCTCACCTCCTAATACTGTTACAATGACAATATTAAATTTCAAC
AAATTTTGGAGGGACATTCAAACCACAGCACAGAGAGAACAGTTTGTGTTCAGGGATTCTGGGATGGCTGGCATTTGTGGAGTGGCATACAAGA
GAGAAGGTATTTCCAAAGACTAAACTCTGCATGGATATGATGAGACTCTACGACCCCTAATAGTTCCTGGGGGTCTTTGAGTTAAAGGAGGTCT
CATGGTCTGGGAAATAACGCAGTTCCAACCAGCCAAAATGGAGAGACATAACACTTTGGGCATTCAGGTGGGATGAGAAAAAGACTACAATGTA
GGAGTTGGGCTACTTTAGCCCTAGAATATGAGCTAATCTTGACTCACCCTAATGAAGCCTAAATCTGAGCTTCAACAGGATTAATTTAAGGTCC
CATCAATTAAATACCACCATAAAGGAACATATTAATAATTCAAATTATCAATATTGCACTTACAATGCCCAGCATGTAATAAAAATTATAAGAC
AGAAAAAGAAGCAGGAAAATGTGACTCATAACCAGAACAAAAAAGATCAATAGAAATGAACTCAGAGATGACACAAATGTTGGAATTAGTAGAT
AGAGAATTTAAAGCAGCTATTATAAATAAATATGTTGGAGGATTTAAAGTAAGATGAATAGATGTAAGTAAAGTAAGATGACCTTAGCAGATAA
ATTGCAACAATTAAAGGAACCAAATGGAATTCTAGAACTGAAAAATAAAATATACCAAAAAGAAAAATTAACTGGATGGGCTTAATACTATAGT
GGATGCAAAAAAAAAAAAAGAAAAAAAGAAAAAAAGAAAGGTCAGTAAAGTCAATGACAGGTCAATATCTCAGAGAGAAAATGCTGAAAAAAAA
AAAAAAAAAAAAGACTAGAGTTTCAGATGATGTAACCAGAGTTTAAATGGACATGAAAGGACCTGGAATAACCAGAATCTTGAAAAAGAATGAA
GTGGAAAATTTACACTACCTGTTTTCAAGACTTAATATAAAGCTATAGTAATCAAGTCCATGTGATATACATATAAGGATAGACAAATATATCA
GTGGTACAGAATAGCACGTTCTGGAAGAAATCTACATATACGGTCAGTTGATCCTCAGCAAAGGCACCAGGGCAGGTTAACGAACAAAGGAGTA
TCCTCTCAACAAATGGTGATGGAACAAATGACAGTCATGAAAAAAAATGAACTGTGACCTGTGTCTCACACCATACACAAAAAATGAGATGAAT
CAAATGATGTAACTGTAAATAATGTTAACCTGAATGTAAAGTTAAAACGATAAGTCTTCTAGAAGGAAATTTAGGACAATGTTTTTGCAAACTT
AGAGTATTTTTTAGACAGGACACAAAGGACTAACCATAAATAATATATTTAGCAAATTGGACTTTATAAAAATCAAAAACTTCTCATCAAAAGA
TACCATTAAAGAAATGAAGGAAGAAAAATACTGGGGAATATATATATGTNNNNNNNNNNNNNNNNNNNNTATATATGTATGTGTGTATATATAT
ATATACACACACATATATATACACACATATATATGTGTATATATATACACATATATGTGTATATATATACACATATATGTATATATCTGACAAG
AACTTCATTTTATGCTAGTATACAAAGGAAAATTCCCTGTGGCCCAGATGAATTGCCATTATTGGGACCCTCTGGCTCACACCAGATTGATTTC
TGATCATGCTTTTGAAATTAGAATTAACCAAGCCCCATCAAGGAGTCTGTAATATAGGCAGGCACCAAATCAGCTGTGTTAAGTACTTCCCTTG
CATGTTGCCCTGAGAACAGGCGAAAGACTTCACTGCCGATTTCTGAAATTGGAAGAGGTCTTCATGGGGTCATCTAGTATAGCTTGTAGATCTA
CTCCAGGGCAAGGATGAACTCAATGGTCTAAATCAGTCTAATCAAACTTGATCTGATCCCTTGGGAGCTTTTTAAGAATCCAAATTCTCTGCTC
CATCCCTGGATCTCTTGATGGAGTAGGTATTAGGTAAGGCCTTGGAATCTATTTTGAGTTCCGTTGGTGGTTTTTTGTTTGTTGTTTTGTTTAG
GCCAGTCAATGAAGCAGTGAGAGTGGAGAAGAAACAAAGAAACCTGTAACTGGTTGTGATCAATTAGTTGTAAACACCACTTCAGTCAGAGCAG
TTTTTGTTCTGCTTTTGTTGTTATTTTTTAGTTTAGTGTGTTGTTTGTTTGAGACAGGGCCTTGCCTCTGTGTCACCCAAGCTGGATTGCAGTG
ACAGTATCAGCTTATTGAAGACTTGAACTCCTGGGTTCAAGTGATCCTCCCATCTCGGCCTCCCAGAGTGCTGGGATTATAGGCACCTAATGGG
TAAGCTGCCACCCCAGGCCCAGCCTTAGTTTTGTTGGTAAGCTAATCAGGAGGTCTTGAAAAGTCTCACCTGCCTCTTAGTCTATCAGAATCTG
ACCCTTCCTTCAAGGTCCATCTCAAATGCTACCATCTTTATGAAGCTTTAGCCATTTCTTCCAGTCAAATCTGACTTTCCCCTTCCTCTGAACT
GCCCGTGTTTACCTCCCTCTAGGCCCTGACTGTACCATGTACTGTAGTATTGCCTGTAGCTTCTTCAGGCTTGGGACAGGGTGCTTATTATATC
TGCAATCCCACAGTACCTATCACAGTGCTCTTTGAGCAAAGAACTCTGGTCTTGTGATGAGAAAAGCCAAGTCTAGGATGCCAGCTCTGCCACT
TGATGGTATTATCCGTGGGTTAGTGATTTATCCAGGGGTGACATTCAGAATATTTATGTGTGTGTGTATATATATATATCAATGAATAAATGAC
AGGGTCTTGCTCTATCACCCAGTCTGGAGTGCAGTGGCACAATCACGGCTCAGTGCAGCCTCCACTTCCCTGGCTCAAATGATCCTCTTACCTC
AGCCTCCCAAATAGCTGGGACCATAGTCATGAGCCACCATGCCAGCTAATATTTTTTTATTCTCAGTAGAGATGAGGTCCTGCTGCGTTGCCCA
GGCTGGTCTCAAACTCCTGAGCTCAGGTGATCCTCCCGCCCCAGACTCCCCAAGTGCTGTGATTACACGTCACCACACCTGGCCTTCAAAATAT
TTAACCAGCATGGCACATGTCCCTTCATTTAGACGCCCGCTGGCCAGGCTGGGGATTAGCCCTGTCTTTATGCTTTTATTCTCGGTAAAGTGGA
TACAGTCCCTACCCACCTCAAAAGGTTGTTGTGATGGTCAAATGAGATAATGCATGTGAAAGTACTCTGTAAAGTACAAAGTGCTATACAAATG
TAAGTCAGGGTTGCCTGGTGGTATTTCTAGTGCCCGTGTGTGGGGTTGGTTGCCCAGCCCTCAACATGGTCTCATGGTCTGCTCTGCTCTGCTC
AGAGCAGTCTCCCAGGAGCAAACCAAGGAGTTGTTGAGTAATTGAGCAGGACACTGCAAATCTGAGAAATCCTGGAATCAGCCTGTTAGGTCCT
AAGGAAACTCAGAATATCAGCTCCTTCAAGGATGGCCCCTTTTTAGGCCTGAAAAATATCAATGTTTCAGTACATATATTTTGGAAAAGCTAAA
TAAAACAAAAACCTAAAAAGGCTTTTTGAATTTTAACCTAAGTTTTCCTTATTTGAGGACACCTTGTTCTATTGCACAATGACCTTAAAATTCA
GGATCCTTAAAGATATCTGGGCATCTCAGACTCATCTGATTGTCACCATATGGCAAGGAGCTGCTCTGCCTAGATCTGTAAATGGAGTTAAATG
CCAGAGACATTAACATAGATTAAAGGGACAGTCTAAATCTTAGTCACTGCTTTGTCTTTATAGTTTCTTGCCTCCTCTTTTTACCCTTTTACTT
CCTTAAAAACTGTCTTAATTTCCTCACCATCGCCTTCCCACATGCCCTCAGGTCTGCAGATCCTGGGGATACAAATCCTTAGCCACTTTTCTCT
TCCAAATGCATTTCACTCTTTCCTCATATTCAGTGCCCAGTTACTGTTTTGTGTTTATCATTAGTTGCCAGTCAATGTTAATGTTTGTTCTTAT
ATGCTTTGATTTAAATGAAGGAAATGGCCCCAGTGTAGTGTGTACAGCAGAGAACTAGAGACTGTAGGGAACAGACAGAAAATAGACACAACCC
TTTTACTTACTCTTGAAGGATTCCCATTTAAAAGGCAATGCAGGGATGGCCACAGTGACTCATGCCTATAATCCCAGAACTTTGGCCGGCCGAG
GTGGGAGGATCGCTTGAGCCCAGGAGTTCAAGACCAGCCTGGGCAACACAGCAGGACCTCATCTCTACAAAATATAATAATAATAAAAAAGAAT
TAGCCTGGCATGGTGATGTGTGCCTATAGTCCCAGATACTTAGGAGGCTGAGGTGGGAAGGTTACTTGAGCCCAGGAAGTCAAGTGTGCAGTGA
GCCATGATTATACCACTGCACTCCGGCCTGGGCAATAGAGTGAGACCTTGTCTCAAAAAATAAAAATAAAAACAAACGGCAAAGATGAGTTCAT
GTCCTTTGTAGGGACATGGATGAAATTGGAAATCATCATTCTCAGTAAACTATCGCAAGAACAAAAAACCAAACACCGCATATTCTCACTCATA
GGTGGGAATTGAACAATGAGAACACATGGACACAGGAAGGGGAACATCACACTCTGGGGACTGTTGTGGGGTGGGAGGAGCGGGGAGGGATAGC
ATTGGGAGATATACCTAATGCTAGATGACGAGTTAGTGGGTGCGGCACACCAGCATGGCACATGTATACATATGTAACTAACCTGCACATTGTG
CACATGTACCCTAAAACTTAAAGTATAATAATAATAAATAAATAAATAAAAACAAAAAAAAAACGGCAATGCAGGACTGTGGTAGATATTAATA
ACTTTAAAACATAATGATAAAATAACCAGGGTGATAATTTGTAGCCCCTTCTTGCTACTTCCTAGCTGTGGGACATTGACTAGATATTTTTACC
TCTCTTGACTCTGTTTCTTCATGTGTGAAACAGAGAAAACATCGATCATGCAGAATTGTAGTAAATATTGAGAGCATGTCTGTAACATGGCCTA
GTTCACTCCTGGCACATGGTGGATGGTTCAGTATATGGTGGCATTTATTATATTCTCATTTAGCATGCAGGCCAGCTCTGGGAAACCCTCAATA
CCCAGGTAGACTATGTGCTTTTTATCTTTACAGCTGTCAGGGTGAGAGGCAGTGGTATCTGGTGGAAAGGGCAGGGTTTACCTCAGAAGACTGG
AACTCAAGTCACAGCACTGTCACTCATAAGATGGGTGACTTTAGGCAAGTCATACCACCTTACTCTCTGTTTCCTCACCTATAAAATAATGCAA
ATAATACCCATCTCTCAGAGTTGTGAGGCTCAGATGAGGCAACGTGTGAGGAGAGTGGTCATTCATAAAACGCTAATAAATAGAGGTTATTAGT
ACTTCAGCACCAGGGTGAGGGTTTCATGTGCCTACGTGACAACTGTCAGGCACCGTAACTATTTGGGACCCACAAGTTAACCAGACATCCGTGG
TGTGAACTCAGTTCAGAACCTTGTTCTCCTCAAAACATATTAACACTTTTTTCTTATCTTGAGTGCCATCTCTGGATTATCTCTCTGTTGGAAC
GGGTGGGCGTAAGACATTATGAAGCTCTCATTCTGCAAAGAGCAAATTGAGGCTCAGAAACAATCAGGGCCATCCCAGAGCCAGGCAGCCTGGG
TTTGAATCTCAGGTCTACTACTTACTAGCTGTGTAAACTTGGACAAATTACCTTACTTCCTTGCCTCAGTTTCTTCATCTGTCAGATGGCACCT
GTCTCACAGGGTTAATATACGTAAAGGCTGTCTTGAAACAGTGTCTGGTACACAGGAAGTGCTCCAAGTATGTTAGCAAAAAGAGAAGACGCGG
GTGGCTAAACCTTGAATGTAGTTCATTTCTTCTGGTTAGATGCTGCATTTCTTCCAACAATCTCAAATATTTCTCCTGGTCTCCCCCATCTTCC
AATAAGTAACAACATTGTTAGTTTCAGTGTTGCATTTTGGCTATTTCTGTAGGACAGTGATACAACACATCAATTCATGACTTAGGATGTTCTG
AAGCACATTCTTTTTTGTTTCATTTTGTTTATGGAGACAGAGTCTTGCTCTATCCCCCAGGCTAGAGTGCAGTGGTGCAATCTTGGCTCACTGC
AACCTCCGCCTCCCAGGTTCAAGCTATTCTCATGCCTCAGCCTCCTGAGTAGCTGAGATTACAGGCCCCCACCACCACACCCAGCTAATTTTTG
TATTTTTAGTAGAGACGAGGTTTTGTCATGTTGGCTAGGCTGGTCTTGAACTCCTGACCTCGGGTAATCTGCCTGCCTCAGCCTCCCAAAGTGC
TGGGATTACAGGCATGAGCCACCGTGCCCAGCCCCTTGAAGCACATTCCTATTCAGCTCTTGACTACTGAATAGAGACCAGTTTCTTTCCTTTG
AATAGTAAGAGATTCCTCACTCACCGGTGCAGTTCTCACTTCAGAGTGGCTCTCCAGGTCCTCCTGCTCACTTCTCTCTAGCACCTCTGTAAAC
TCTTGAGGGTTCTGGCGTTGGGGGAGTGCGCTTAAAACCTAGGATTGAAGGGAAGCTTGGAAGGCTACTAATATAGTTGGGATGTTGTTCCTGC
CCAAATCTCATGTTGAAATGTAATGTTGGAGATGGGGCCTGGTGGGAGATGCCTGGATCATGGGGGTAGGTTTCTCATGAATGGTTTAGTACCC
TCCTCTTGGTACTGTCCCCATGATAGTGAGTTCTCATGAGATATATGTCCCTCTGGGAAGCTAGGACAGGGCCCCACTGCTCCTGCTTCCCTTG
CCTGGCTCATGTCTATAGTGCTGCATGGGAATGGTCAATGTCTATGAGCCATGGGGTTGCGTGCATGGCAGCTCATCAAAGAATCAAACTGTAG
CATAACTACCCAGCCAGATGTGTGGCACTTCCTCCCTCTCCCTCTTGCTCCTGCTTTCACCATGTGACGTGCCTGCTTTTTCACCCTCTACCAT
GATTGTAAGCTTCCTGAGGGCTTCCCAGAAGCAAATGCTGCTATGTTTCTTATTCAGCCTGCAGAACCTTGAACCAATTAAATTTCTTTTCTTT
ATAAATTATATTTTCTTTATAGCAATGCAAGAATGGACTAACACAGCCACCAACTAACTGCCAAGGAGGACCCCATGCTTGAGGGGTCCAGTCT
CGCTGGCACAGTGATGAACTACAGTGGACAGCCACTCCTTGAGTAGTGGTCATAGTTAAGGGACAAAAAAATCATGGTTCCAGTCTGGAATAGC
CAAACAAAGAAAAAAAATAGCCAGAACAAAGGAAAAAAAAACAAAATAGTGCTTAAATCTAGCAAGAGAAATAAGAATAATTCATCACTGTTAT
AGTGCTTCACACTTTTCTAAGCATTTTTATGTATTTAATCAAACATCATCTAATCCTAACCACAACCAAAGAAGGGGTGAGGAAACTTTCAGAA
TAATTTCTATCTGATTTCAAAAGACTCTGTTCAGTTACTTTTTACCCTACATGGTGATGTGATGTTTTTTTGTACGAGACTGTTCATTGCAGAG
AATTATAAGCAATTAAAGAACAGTTTCTGTGGTGAAAAGCATATTTTACAATAAGACTATGGTCAGTGGATTTCTATTTTATGTCAGCATACAC
TGTTTCCCACAATTCCATCATGCACTGGAATGAGGTTATCTATTATCTAGGGTTGTGGGTCTGATGGGACCAAGTCACTCATTGAGAAAAATTG
TCTGAGGAAGGCTGAGGTCATGGCTAACCAGTAATTCTCAGATGGGCCTTATATTATTTTGTTGTGTAAGTATTTGGGAAGTTCTCCAAGTTGC
TTCTTCGTGCCTATTGGGCATGTCTGAAAATATGCAAAAAAGGCCAAGAATTTAATAGATAAGAATGACGTGATACAAATATGCTCCAGTGAGC
AAGGTAAGAGCCTGACCCAGATTTTCTTCCCAGTTTGTTTATTTCTTTTTCTTTGGGTCCACTGGCCCTTTGCCAGCCCTCCTCCCAGCAAGCT
GCTGGCAGCTCTCTGTGTCCTTCTGGGAAGCTAGGACAGGGCCCCGCTGCTCCTTCTTCCCTTGCCTGGCTCATGTCTATAGTCCTGCATGGGA
ATGGTCAATGTCTATGAGCCATGGGGTTGCGTGCATGGCAGCTCATCAAAGAATCAAACTGTAGCATAACTACCCAGCCAGATAATTCAATTGT
TCCTAGAGAGTTTGATGGGACCCAGAAGAAGTAGCCACCAGGTATATTCAGTCGGCACTGACTTCAACAAATGCTCTTAGAGTTTGAAGTCATA
GCGTTGTCCCAGAGTCAGCTCACTGGGTCTGCTCGGGACTTGCAGTACCTCTTCCCTGCTCTTCTGTTTATTTCCTAGCCCCAGTCCCCAAACC
CCACCCACAGACTTGTGTCCAATTTCCAGAACTGAAGAGGACCAACTTCCCTCCTGACTTCTGAGACTCATGATTCTTCAGACATGACAAGTCC
AGATCGAGACTGCAATCCCCAAGGAAGGCCAGCTGTCTTGCTCAGCAGAGACAATACAGCTAAAAGCTCTTTCTCACTTTAGGCAATTCATGCA
CTTGGTTAGACATTTATAGACTTCAACTGCTCATCCAAAAAATCTAGGTCATTCTTTACATAGAGCTTCACACTTTGGAAGGAAAGCCAGGCCC
AGGGAGGAGCGTTTCTGTTGATGTCTGGTTTGGCTGAGGCAGTAGAGAGTGCTTCTGCTGTGGGAAGAGGGAGAGATGGACCAGCGCAGGCACT
GTGGGCCCTTAGGGGACGTGAATTTTCACCCCCTGGCCACTGACTCACAGAGTCTCTGAGTAAATGGTGCCAATAATAGCTGCCTTGTGTTGAT
GTCATGAAAAGTGGTGGGTTTAACGATTAGGCTTTCTAAGGACATTTTTAAAGTATGTACCATAATTCCAGTGAGAAGGGCTGGGTGGGTCTGT
TGGGGGTGTAGGGCTTCCTGGGGAGGAAGAAAGAATAGAGGTGTGGGTGAGAGGTTTAGCCATGTAACATGGATGGCCAAAATGGTACAGCTCT
TCCAAGTATATTCCTACCTGCCTCCTAAGCCCCGAGGAGAAGGACATGCCAAAGAAAGAGCCTTGGGCTGGGAGTGAGAGGGCCCTTCTTGTGG
AGGTTCCTCTACTCACCATGGCTGAGGCATGTTGCTTCAGTTTTCTCATTTTTAAATTTCAGCTCATGGTTCTGCTGCTCCATTGAGCTGCTGT
ACAATGCAAATGTTCAACACAAGTGAAGCTGTTTGTTGAACCTAAGTGCCACAAAAACGTAACATATCATTATCAGGTGTTCTCACCCAGAGAG
AATAGTGTGGTTCTTCAGCCACTCGCTTGCTTCAGGGATGGTTTGTGTGGGCATGTGCTCATTTTGTACAGGTCTCTTCCTGTGTACCTAGATG
CTGTGCAACTTGAAAGGCCTGCTCCTCCTTCCTGCTCTACTACTCTGGCCCCAGCAGGACAGTGAATATCAATAGAACAGAGGCAGGAAGCATG
GGTTCTTTTCCTTCAAGTCTTTTCCACGTTGGCTTCTCTGCAACAGGAGTTGAGGGAGGCTTGGTTAGAACAAATCACCTTTGTCTGGGGGTGG
GTTAACCAGATTTTTCCCTCTTCTGTGTCTGTGGCCTTGGTTGTGGCACCAGCTGTCATCACTGCCCCACCAGTTGGGTGGGAGAGAAGAGATG
AGCCTGGCCAGCCCCTCTCCTGCTTTAGTAAGACTCCGCTTAAAGGCCTGTTGTAAAAGTTTGAAATTGCAGGTCAGGGTTAATTTAAAGCTTG
TGTATTGTCCATGTTATGGTGTCCCCCAGCCATTACAAGTAAATAATTAAAAGCCAATTCTAACCAAGATATTCAGAGCTCCATAAATTCCTGG
ACACCATAATATGACAAGAAATAGACACTAGTCTAACTATATGGCAGGCTGTCCTATGTGGGCCAGGGTATGACATACCAGGTTATCCCAGAAA
GAAGAACAGGACAAGCTTTACAAGGAACTGAGCTTCCCTTCTCCCAAATTCTCACCTTATATTAACATTTTTGTTCTTGGTTTAGAGCACCAAC
CACTTCAAGAAACTGCTGTTGCAATGTGGATTTCTCTGAGAGGATTCACCCTTGAAGTCCTTGTCTCCTCCAGAGGAATTTGTTGATCTGGATT
AACTGTCCAGTCATTGGGCAAGGCCCAGACGGGATGAGATTAGGAGTGAGATGAGGAGTGGAATGGGTTCAGGAACACCTGGGGAGCAAGGAAA
CCAGACAGGAAGGAATACAAAAGGAAGCCCAGCAAGCACCAGCCCTCTTGAGGAGGCTTGCAGCTGCTGTGGGTGCATGCTGGAGGTGGCCCAC
ACCTGTCCTGAGAGCTGACTGCAAGCATCTCTTCCTAGGTGCATGTTCAGTGACTTCATGTTGGTAGCTTGAAACTGGCCATAGTGGAAGTATT
TACACCATGCAAATACGTGAACACTACAAACCAGGGTTCCTCTCACTTCTCTCCCAGGAGAGCTTTACATTAAACTCTTGCAAGCATCCTGCTG
GTGGCAACCATAATGGATTCTCACTTTCCCTGAAAAGCAGGACTTAATCCTTAGCAGATCACTTGCTTTCTCCCGATTGTTCTCCTCCCCTGAG
TCTTGAGGGGCTGGGGACTAACAGAGACCAAAGTGGCCTTGGTGATATGTATGAAGTGGCTCAGGTCATAGACGGTATACAAGTTCAAAGTTTG
TATCACCACATTGTTTCTTCACATGCACTTCCTGCCTAAGTTGCTGCCGTCCGCTCTAAAAAACTAAACAAACAACGACCAGAAAACAAACTCT
ACTGATCTTATTTTCAGCTTGCCAATTTTTATACAGTCTCCAAACAGGATTTAATGAATGTAATAATCTTCCTGACAAATAGGTTTTACTTCTT
TGGAATATTTATCACATAGTTATTTATTTAGAATTCTGTGTGTATACCTACCAGGTTCAACCTAGACCCAGCCTAACCATGTTTCTGGAGATAC
TGACCTCCATTATCAACCTCAGCCTTACCACAGGCTCTGCTCTAACCTTGCCCCTAGGTTTTCTGATCCTAGCTTTCCCATTACCACCCTCTGG
CCTCTGGTTGTGGCACACCTGTTATTTCATGTTAGGCCCTCAATGCTGACAGCTTGCCTATCCTTCATATGCTGATCTGTCTCTGGTACACTCA
AAATACCTWCACACATCAAGAGATTTCCTTGGAAACCAAAACAAAGGAGAAGAAGTGACTGTTCCGGAGTTGCTGCCTGGTGCTGGACGTTTGT
TTTCATGTTTCATGTTCATCTTGCAGCAAGGCATTTCCATCTCATCCCCCAGACAAGCCTTCACTGTAGGAAAGCTGGCCCCCACCTCTTATCA
GCAAGGAGCAGGGTCTGCATCACCACCCTTTGCCAGTTTTCTCTCACGAGACTCTGATCATCAGGGAGCAGGTCTTCTCTCTCAGAGCCAAGGA
GCTTATTTCAGGCAATAACAGCAGTTTTCATATGGTTCTTCACATCTCCTTTAGTACCTAGGACAGTGCTTTCCATAGGGTAGATTCATTATCA
ATGTTGGTTAGTGGTGTCAAAAATTATTTCTACTTGAAGTAGAATGAACAGTTGCTATATAGAATAGTTAGGAAGCCATATCCTGTCATTGGAG
GGGTCCAAGTAGAGGCCAGACAACCACTTACCTGTGATGGTTTTAGGAGGGAATAATGCATTTGGATAGAAAGAAGAGGTAGAAGATGGCTAGC
CTTTTCCACCCTGATTGTGATCTCATAAACTATGGGATGCCAGCACTGAGTCTAACGGTGGAAAATAGGCTGTTGGCCCAGAGACAGATGACTC
TCCAGAATCCGCTTAGTTTTTCTGTTTTCTTGAGCAAGTGGTCTGCAGGTTGAGTGAGTCTTTAATTAATATGCTTCTCTAGCAGCTTACATTA
CTCCCCTAGTCAAATGGCAGAGTATGTAAGATCAACATTGTGTACCCCACTGAGGCTTCAACCCTTTAAAGAAGTCTTTTCCTGTAAGTCTAAG
GAGAAGCAGATGGTAATGAAAAAATACGCACATTTTATTATGCGTTGCACATTTTACTTATTAGAATTTTTAGTATGACACCCTGCATTTTAAA
AAGCCTAAAGATGAAAACAAAATGGAATTATTCTTTGAAATATTTAAATCAGCTTGCAGCAATTATCTCAAAATATGAAAATAAAATCTAGCAG
CCTGGAAGGTAGATCTTCCTTGACCACCCACTTTGAAAATCTCGTCTTTCAAAGGGAGGCTCTTCCTCTCAAAAAGGAAGAAGAGCATGCTGTA
CAGAATGAGGGCAGGAAGGGACGTATCCCCGATGAGGTTGTTCAAAAAGGAAGAAGAGCATGCTGTACGGAATGAGGGCAGGAAGGGACATATC
CCTGATGAGGTTGTATAGAGCTGGAGCTTATGTGTGGGAGAGCTGGTGGTGGGAATGAGGGCAAGTGACTACACTGAACCATTCTCTCGTTGAA
AAGTAGAACCACGACTGGAGAGGAGTTACCTCGGAGTTCTGGACTGGGTGTAGAGAGAGAGTGTTTCTGTCTCCTCTTTCCCTAACCACATCTC
TGCCTCCCTTGGCTGAGTGAGTCATCCTCTGCCAGCCTTACCATTTGCCAGCACTCACTCCCACAGAGACCATGTGAGGATTAACAAGCCGTAC
TTTGGGAACGTCACTGAAACTGTGGAAAAGCAGTGCAAGGTAAAGGTGAAGTGTGAATATGATTGTGAATCTTAAGCACAGAACCCTGAGTTCT
TAAAGGCAATTTTTAAGATTAGTATCCATCAACCAAAGTTCACAAGAGTTTTTCACAAATCACCAGAGTACAAAAGCTATTTCACGAAATGTGC
AGTGGCTGTGACCTTAGTGAGATTTCTTAGACTTCTAGCCATAAGGACTGGAAGAGTTCGTAAAGCTCAGCTCCCACTTTATTCTTGAATTCCT
TATCCTGACGAATGGTGATTCAGCATGCAGCCAGGTATAGTGGAAAGAGAAGGGGCTCTGGAATCAGACAGACCTTGGGCCAGGTCCCAGCTCC
TCCACCCATTGTCCCTTTGACCTAGAACAAATTACCTAATCTCTCTATGCTTTGATTTCCTCAGCTGTGAGTTGTGAACAATGCTTGTTGTTGA
GAGTTAGAACATGGTTAAAGTGTTTAGCACAGCTCCTGGTATTTAGTAGGGCTTCAATAAATCATAGCTGCTATGCTAATTACAATAATGCTTA
TTAAGTGTTGCTCTTCAGGCTCTTTTTTAATATCTCTGTGATGGCAATGCTTCCATCATTAGACAGTGCTGATGGCTAGAATTTTCTGCCACAT
GTTGTGCCCAATCCTATTTCCTATAATTTCTATCCATCTACTCTGGTGCTGCCTTTTGTGGCCCCATAGAACAAGTCTAAACCAACTCCCACAA
AGAACACTTCAGATATTTGAAGACAATTTTAATCTCTTCCCCAAGTTTTCTTCATGGTAAACCCTTTAGTTCCTGGAATCCTTCCATAGGTGAC
TTAGTTTTGATCTCACCCAGGACACGTCTAAGTCCCTTTTCCTCACATATGAGTATCTATGGGAAGATGCTAGCCAGCCATAAAGGGAAAGTTG
AGATGGCAAGTTTTAGATGCCATCCATACTGAATAAGCTGGCTTTTGTCCATGGTTCTTGTGAACATTCTAATGCATACCCTACAAGTATATTT
CACAGTTCCTTCTTTTCACTGTAGTTTCACCAGAGTGTTCAGGAATATGAACACTGCTACCTGTGAGGAAGNNNNNNNNNNNNNNNNNNNNTTG
CAGTTCCTGCCCTTTGACTTTATATTCAGTAGTGTGACTTGCTTTGGCCAACAGAATGAGTCAGAAGAAACAGCACGCCAGCTCCAAACCTAGG
CTTCAGAGGCCCTGGGTATTTCCAGTTACTCTTCAGTGTTCTGCCATCATCATGAGATAACCTACCCCCACTAGCCTACTGGCCCAAGGAAGCA
GGGAGGCATGTAGGCTAGGGCCCCCAGCAGCACCCAGCCTGCACTAGCTGATTGTAACCAACTCACAGAAACTTGAGCAAAAGTAAGTGATTAC
TGTTTTAAGGCACTGAGTTTTGGGGTGGTTGGTTATGCAGAATTTTTGTGGCAAGAAATAATTATTATGGAAATTATGTGGGGCCAGAAAACAT
AAAGTAATACTAACCCTACCTGGACAGAGTACAAGTTTAGAAGAATTAATGAAAGATTTACAAAACTATAACCAAGCTAGATGAACACTTTTTA
AAAATGATTTACCAAATTGTAACTTTGAATTTCTTGTGCAAACTGTGACCAGGAAGAAGATAAATGTGTATATTACATTTCACGAATGAAATAG
GTAATGACGAAAGCAAACTCAATTTCTAAATATCTGTGAAAAGTTAACGTGTTTCCTATTCGATATAGTTCACTCATTTACTCAAGAAATGTTG
AATGAATCATGTGTCAGTACCATTCTAGGCCCTGGAAATATAGTTGTGGAAAAAGTGGACAAGGCCCAGGTCTCATGAAACTTGTATTCTACTG
GAGATTTAGAAAATAAAAAATTAAATAAATAAAGCAGATAACTTCAGATAATGGTAAGTACCATGAGAAAACTCAGGCAGGGTAAGGAATACCT
TAGGTAGTTTAGAAAAGACTTCTCTGAGGATGTGACATTGAAACTAAGACCAAAATGACATCAGTAATTCAAGGGAAGAAAGACTACATTGAAG
AAAATAGCAAATGCAAAGGCCCTGAGGTAGGAATAAGCTGGGAGTGTTTGAGGTACAATTAGAGGTGTGGAGAGGAGCAATGTGGTATGAAATG
AACTGGATGATATTTAAAGAGGCCAGGACATGTAGTGCTTTTTTGGCCACAGTAAGGAGTCCGTTTTATTCCAAGTCCAATGGAATCCACTGAA
GTGTACTAAGTGCATGACTTGATTTATAGGTTTTAGGAGTTTGTCCTGACTTCTGTATAGGGAATAGATTGTAGGTGGACAAGAGTGGAAGGAA
GGAGGTCGATTAAGTCATTGCATCATGGTGGCTACAACTAGGGTGAGGCCAAGGATGGAGAGAAATGAACAGGTTTGAGATATATATTGGAGGT
AGAATCAAGACTTCCTGATGGATTGGATGTGAAGGAACATGAGAAATAAAGGATAATGGCTAGGTTTTTAGTTGGGGTAACTGAGAGGATGGTT
GGTGGTGCCATTTCTGGAGATGAGGCAGATGCGAGGAACAATGAACTCAGGGGAGGGAGAGATCAGGAGTTTTGTTTGAATGAGTTAAGTCTGA
ACTTAGACGTTGAATTGGATTTGCCAGGTAGGCTGCTGGACATACTAATTGGGTAGCGAAGGCTGAAGATACCAGTTGGGCAGTCATCAGCATA
TAGATGGTATTTTAGCCAAGAGACTAAATGAGTTCATCTAGAAAATAGTAGTGAAAGTGTTTTATTTTTAATTTAAAATTTAAGAAGTTTTAAA
TTTTAAAAACTGTCAAAAAATAAATAAAAGAAAAAGCGGGAGGACTCAACCACAGAGCATGCTGACATTTAAAAGCTTAGCAGAAAAAGAGGAG
ACAGAAGAAGAGCCTGAGAGAGAGGCTGAGAGAGTGTGATGCTATGGAAGCCAAGAGAAGAAAGTGTTCCAGGAAAGAGAGTCATCAACTTGGG
TTGTGTTGGGTTGAATGCTACTGAGTCATAAAGTAAGATAAGGATCAAGGAGTAACTGTTGGATTTGGCAGTGTGGAGCTGATTGGTAACTTTG
AAAATAAAAGATTTAGTGGAATAATGGACACAGAAGTCAAATGGAATGATTGAAAAGTAAATGGGAGAAAAGAAACTGATAACAGTGAGGTTTT
GCTAAAAATGGGAATAGAGAAATAAGTAGCTAGAGAGGATGAGAGTTTAAGGTTTTGTTTTATTTGGTTTTGAATTGGAGATGCAGAGGCATAT
TTCAATGACAATGGAAATAAACCCACAGAGAGAAAAGTTGATATTGTGGGAGAGAAAATGGACAAATGCAGGATCAACGTCCTTGAGAAAGGGG
GAGACCCAGAGCACAGTGAACTGTTGGCCTTCCATGCTAACAGGAGAGGGCAGGGGATAAGGGCAACAGAGTACCAGTGAAATGGTGGATTTGG
GGATGGGAAGATGAAAGACACTGTCAGTTTACATATACATATATATATACATTTTAATATATACACTTTAATTATTGAGATGGGCTCATGCTCT
GTCACCCAGACAGAGTGCAGTGATGTTATCATAGCTCACTGCAGCCTCAAACTCCTGACCTCAAGCGATCCTCCCACCTTGACCTCCCAAAGCA
CTGGGATTACAAGCATGAGCCACTGTGCCCAGCCACTTATATTTTCTCAATGAGATATGACTTTAGATCGTCGTTTGAGGAGCCAAGACTTCTG
AATCCTGAGCCCAAAAGCACTGTGTCCTTCATTTACCATTTGGTACATTTGAAGCAGAGTGAATATGATAACCCCTTTAATATAACTTTAAAGA
GCAATACAGATGACAAAACAATTCATGTATATTCTTTCATTTGATACTCTTGCCTTGAATAATTGAAGTTCCCAGTCTGGAACAGAATTCAGTT
CGTATAAATGAAGACATTGTAATGAAGGGACTATTTACAGAGATGAGAGTGGAATTAAGAGATGTATCCAGAAACTATCAACACTCAGGAGTCA
TTACCACCCTTGGGGCCAGGGGGCCAAAGGAGATTAATAGTATTAGATCCAACGAGAGCTGGTGCCATGAAGGGAGGACCAACCAGCATGATCA
TTCATTGAAAAGATATGTAGTCACTGTCAGGGGAGTGGTGTCAATGCAGAGAGGGAATGAAAAACAAATATCCTGACTTCCCTCCACTCCCAGT
CTCTTGCTGGTGCTTCCCAATGGCTAAACCCAACTGGAAGCCAGCTGGCAAGTGAGTGGGCTGATATTTTTCATGGTGAAAGAATAGATCTGGC
AGAGAATAACCAGCAGAGCTCCTAACACTTCATGCCATAAATCTTGAAAAGATTTTGAAAACCACTCCAAGGGGAATCTTTTTACCCAATGCCA
GTGTATAAATGCATCTTATTATTTTGTGCCAGCTGGGATTTAGAAAACCTAGCTAACCATGTTCTTTAATACAAATCATCATGAGTACTTGTAA
TCCACTTGCAATCTTAAAATGTAATGTTTGCCCATAGTCTTCTTTTATTTCCATCCAGAGGCATATTAACCATAAAACTGGAGAAGCATATATT
TCAGGGCTCCTCACTTGCATGAGCCCCTTCCAAGGCCATGGGACGGCCCTAGAAATGTATTCATATGGTCATTTGTTTTTGTAAAATTTGTAAA
GGTAAGATATTTTAACTGCAATCTATTAAAAACCAACTTTTCTCACTCTGACCCCAGAGAATTCTCTCCCTGCCCACCCCCCAACCAGTTATAT
GAACTTTAGGCCCCACAAAACCTGGATCTCCTTCTGCTTTCAGTGAGACTCAAGTGTGCTGATGCAGATTTTATCCCTTTCCCGATTTTACCAC
TGTGGAGAGGAAAAGGGGTAGTTAATGAGCATCAAAATGGACATTCTGCAAGGGAAGAATGTAGCAGGAGGATGAGAAAAACAGAGGGGGGCTG
TGCTAGGGGCAGTGGAAGAAAATCACTGTGCCTGAAGGATTGCACCTGAGTCACAGAGGGTTTGCAGGTGTCTACCAGGTGACTAGTTTCCCTC
CAGGGCCTAACTCAGGTGCTGCTCTTTATATTTTCAGATGCAGATGGAAACCTGTGTCGAAGTAACCAACTGTTGCAAGTCATGCTGACCATGC
ACCGAATTGTCATCTCCTCTGCAGAACTGCTCCAAAAAGTTATCACCCTATATCCTTTAACATAGTGACTGGCAATCCCAAAATGGGATCCACC
CAAAAAGTAGCTTGAAATTTGAAGTATGCTTTTATTTTTATTGGTTTGGAATAACCCTGAGTGGCAAATAGGTTCATATGTCCTTTCAGGGGTA
TGGGTGCATGTGTTGGGGGTGTGTTCATGTATCCTCTGTTGGGTGGGGCAATGTTATTGATGCTGATTGCTCTGGGGGGTGGTCAAAGAAATCA
ATATGCAAGCACAATCTTCAGGCTCTTAATAGTAAAAAATATTTTTTCTGTAGAGTGTGCAAAATAGATATTCATCCTGTGCAAATTGACAAAG
GTGCAAGCATGACTCAAGGATAAATTTCTTTCTGGCCTTCCTGTCCAATTCTTCAACTTTCTCCAGGTTCTGAAGAAAGATTCCCAGTGCCACA
GCATCTCGAGAATCTGCTGGTGCCTTATTGCTTCAGGCACGAGGACTGACTATGTGCCAAGCAAAACCTGCTGGACTTTAACCTTATTACGGGA
TATGAATCAAGTAGGGAGGAGAAAGCTCCTGCTTGCTTTTTCTCACTGATGACACTGTAAGCAGCAGCCCCCACAAAAGAAGAGTTCTAGGCGG
AGTGAGACCGTTTAGCACCCAAGGCGCAGGTCCCTGGTACTGTGTCATGACTTGTAGCTGCTCTAACACTTTTCCACTCTCCTAATTCCCCGCG
TTTCATCTTACAGCACACAGAAAAAATAAAAACCATCAGGACTGAATCCCTTCTCTATACCTACTCATGAGTCTTTACCTCTCATCTCAAAGGA
GGTGTTTTCCAGTAGCACACAAAGTGGGGGAGCATTGCGAGAAGTGCACATAAATAAGAGGTGCAGGTAATAAGTGGGTGCCTCTTCTACAGAG
AATTTTAAACCATCATGAAACCAACTCAGTGTCTACTTTTTATTAATTTCATGTACCAGTGATTATAAACAATGTCAGTGATTAAATATTCCTC
CCTAAAAACTTATTTGTTGGCCTAGGGACTAAATTGTTCCTGGGGTGTCTGGGCACAGTAGCTCACATCTGTAATCCCAGCACTTTGGAAGGCC
AAAGTGGGCAGATCACTTAAGGTCAGGAGTTTGAGACCAGCCTGGCCAACATGGTGAAACCCCATCTCTACTAAAAATATGAAAATTAGCCGGG
CATGGTGACATATGCCTGTAATTCCAGCTACTCATGAGGCTGAGGCAGGAGAATCACTTGAACCTGGGAACTGGAGGTTGCAGTCAGCTGAGAT
CACACCACTGCACTCCAGCCTGGGCAATGAAGTGAGACTCTGTCTCAAAAAAAAAAAAAAGAAAGAAAATTGTACCTCGGGGTTCCTGCCCTTA
GTGCACTACTGGCTGCCTACATATTGGGGCAGACCCCCCCTCCCTTTCCCATGGTCCCTCGTACTTCTCACAGAATCTTACTCCACCAGCTATC
ACCCCTCCTATACTTTTAAACTCTACCTCTTCAATGGCACCTGCCCCTCAGCCTAAAACACATTCAAGTCTCCTCATCTAAAAATCAACTCTTA
ACCCTACCTCCCCTTCATTCCATTGTTAGGGTTAAAAATTTGATGTGAAAGCGTCTTAAAATAGTCTACACTCACTATCTCCAATCTCTTTACT
ACTCCTCACTTCGTAACTGTGTTAAGTTGGCCCCCAGTATTCGAACTTTCCTCTGATGTCACCACTAACCTCCTGATTGCAAAGCCCAATGGCT
TTTTCTCATCCTTGTTTCCATTGACCCCCAGAAGCATTAGGCATGACTGACAACCCCGTTCTCCTCCCAGCTCTCCCTCCAAGGCTCCCGGAAC
GACACCCTCCTGGCTTTCCTACCTCTTGGGTTGGGCCTTCTCCAGCTTTTCATCTAGAGGCTTCTTCGAGCTCTGCTTACATATGGTGTAATGT
CCTGGTTTTCCTCTTACTCAGCTTCCTTTCTGTGGGTGATTTCTCACCAGAATTCCTACCACTGTTTCCACCATTCTTCTGTGCTGATGGTGAC
TTTCAAGTCTGGATTTCTCTAACTTAAACCTTTCCTTTTTTTTTTGGAAATTGGTGCGGGGAGAGGGGTCTCACTATATTGCCTAGTCTGGTCT
CAAACTCCTGGCATCAAGTCATCCTCCCACCTCAGCCTCCCAAAGTACTGGGATTCCAGGCATGAGCCACGGCACCCAGCCATCTAACCTAAAC
CATTTTTCTTAGGTTTATACCAGTTTGTCCTGCTAACACTTAAGACTTTAATATATGCATTAGCCCACTGTCCCCACCCACAGCCACTACCTCA
TTAATTCTCCTTCGACACCAGTACAGCCTTGTTTTGCCTAAGACACATTCTGAGAAATACATCATTGGGCGACTCTGTCATTGTGTGAACACCA
TAGAGTGTACTTACACAAACCTAGATGGCATAGCCTGCTACACTTCTAGGCTAGACGGTACAGCCTGTTGCTCCTAGGCTACAAACCTGTACAG
CAGGTTACTGTACTGAATACTGTAGGTGGTTGTAGCACATGGTAAGTATTTATATACCTAAACATAGAAAAAGTACAGTAAAAATATGGTATAA
AAGATTAAAAAATGATATGTATAGGGGGGCACTTACCATGAATGGAGCTTGCAGGACTGGAAGTTGCTCTGGGTAGTCAGGGGTGAGTGGTGAG
TGAATGTGAAGGCCTAGGACATTACTGTACATAACTGTAGGTTTTATAAACACTATATACTTAGGCTATACTAAATTGATAAAAAATATTTTTC
TTTCTTCAATAATAATTTAGCTTACTGTGACTTTATGTTATAAACTTAATTTTTAAAACTTTGACTCTACACAGTGTACAGCTGTGTAAAAATA
TTTTCTTTATATTCTTCTGTAAGCTTCTTTCTGTTTTAAAAATGTTTAATGTTTATTAACTTTTTAAACTTTCTTGTCAAAATCTAAGACACAA
GTATACACAGTAGCCTAGGCCTATATTGGGTCAGGATTATGAAGACAGGATGAGGCAATAGGAATTTTTCAACTTCATTGTAATCTCATGGGAC
CACTGTCATATATGTGGTCCGTCATGGACCAAAATGTTATGCAGCACATGACTGTATTTTCATGGTCAATTTCAGTTAATAGTGTTAGTATCTA
TTCTTGCCTAAACCAGTAACATCAGAAGCACTCTTGACTTCTCCCCTGCTTTCACCTCCTACAGTCTGTCACCTGATCCTAGGGTTTCTACTTC
AGAAATGACTCTTATCTGGTCTCTGCTCTCCATTCCCATACCATGCCTTAGTTCATGTCTCCGTGGTCTCTCTCCTGAACCACTGTCCCACTCC
AGTCTTTGTATCTACATGGTCCGCCTACCCCGTCTCATGGTAGCTACCCTTGAAATTCTTTCCTACAAAGCAAATTGGGCAGGCCATTGTTTCT
GACCCTCTGCAGACTACGGAGTTAATGTCAACTCCTTAGCTTGCCAGCAAAAGGGCCTCCTCAGTCTGTTCCCAGCGAGTTTTTCCTGCTTCAT
TCCGTAGCTCTCTGAGAAACCCTTTCCCTTCCTCACTGAACTTCTCAATATACTGGATGTAATGGGGCCTTTTATCATTCCTGTGCCTTGAAAG
CCTTTCTACCCTCTTTGTTTAGAAGCTCTTTCAAGACCCTTCTGGGAAGCTTTTCTAGACTTTTCCAGCCCTTCTCCCCTTTTAAAAATACTTC
TAATGTTTTAAAATAGTATTATTGATTGACAATCTATTTAATTCCTTTGATTTTAGTTTTGTACTGCTTAAGTCAAAGTGCGTTTCTCATATCT
CTGGCCTGGATGTAATCTGCATTGAAAAACTCACAGAAAGTGTGAAGTGTTTTTATTCTGTTAGCTAAATAAATGTATCTACCTAAACTTCAGT
CCGTTTTGCAATGTAGTGCCTATGTACTCTGGTCTTTATAGATGACCGTTTCAAGTAAAGTACAGAACGTTTGTGTCTAGCTGCTGTGATTTCA
ATTTGGTTAATAATATAAAAGATTGCAAGGGTACTGAAAAATTGATTGAATTACAAATTAAGAAAGATATATTTTTGTATTTTAAAAGTATGTA
CACTGTAACTTACCTATTTGTGTGTCGTCTCCCCCGCTAGACTCTGTCTTTCAAGGGCAGGGAGCAAGGCTTATTTATCTTTGTATCTAGAACT
TAGTTTAGTGGCTGTTTCATGGTAGGACACAATAAATGTTGCATTACTATGGGTGCAAACATTGGGATACTTTTTATATTTACAAGAACTTTTA
CCTGTGATATCTGGGCTTTGCTCAGGTGAAGATGTAACCTGGGGTAGGGAAAGGGATTCTGAGAGTATGAAATAATACTCCTGTAATGAAAGCC
AGGTTAAACATTGAAGGCAAAATTATTTTCTTTCTTTACGCTTAGTTTCCCTGTTCTCTGACCTGGAAGTAATAGAATTTTTAGTTAAATAAGC
TCAGATGGTCTCCTGATTACCATTGTGAAAAGAAGAGAGGGATAAATTTGAAAATGTGCTTTGAATAGTTTAGGTGCCTGAAAAGTACAGTCAG
AACTGTACTGGTACTTTATAGCCACAGTACTAGCTATGAAGAAAAGAAAAAGGTATGGGGTCTGAGGGTTCCAGCAAACACTGAAGTTTACCAA
GATCAGCAGATGCCTGAGCCTCTTACTATTTAGCCTGCATGTGCTGGTGTTTGCTTCAGCTCCTGAATGAAGTGAGAAAGAGGGTGCCCTGCTC
CCTGTGGGGGGCAGCCTTTGCCATGGCATAGGGGCTCTAAGGACCTCTGAATGATGCCCAGGCTCAGCACTCACGTGTAGACTGACTGGATTTA
CAACACTGACGCTGCACCACATGCAGCCAACGTGCACTGTCAGAATATACTTTTGAAGGGAACGATAAGGAGCCACTGGGAGAAGGTGAATGAC
ACGGTAAAAGTCACCATGTCGATTTAATGTATCTACTTAGCTTTAAAACAGAAGTGTGGTTTTTGTGCCTTCCCCTTTAAAATGGAAGAATAAA
AATTTCAACAAGTAGAACAAATGATAAAAATATAGAATCTGAGAGGTATTTATCTATGTGCCTTTTGCCTGAATCTCTTTGCATTGAAGTGATG
CATTTTCTTAGTGATGTGGCATAAAGAAACATAAAGCCATCTCATTCTCACTTCTACTTTTCCACCCAAAGAAAGGCAGGCATAAAAACTTTCT
ACTTCATGAAGTTTGCCAGTTCCCTGCACTTATTTTTATGTCAATTACCTTTGTTGATGTGTAGGGTTTTTTGGGTTTTTGTTTTGTTTTGGTG
CTCTGATCTAAAGGGCAGATTTTATAGGCAGTATATGTATTAAAATTTAATTTTTAAAACTAGAAGTCATTTTATTAAAATTACAAATTTTATA
ACCATAGTTCTTGTTTAGGGTTTCAAGTTTAGCTTGCAAATCTTACTGTTTCCATAAGTCTAGCATGTTTCAGCAAGCATTTTTAAGGGACTTT
GAAAAATGTTTCGTTAATTTGAATAATGTTAATTAAATATTCCCAATGATTTTAAACTACAGTTCTGCATTTTATATCTTAGATAATTTTTAAG
TGCTTCAGATACCTCCCTGAATTCTAAATAGTTTTTTATAACTCTAATAGATTAAATTTATAAACAGTAAAGCTATCATTATCTTAGATGTTCC
AAAGCTATGCATAAACTTAATAAGGTTTCAATTTAAAACCACAAGTGTAAAGCACTTAATTATATATTGCAAATTGAAGTTTCAAGGCTATCAT
TCTAATATGCTTTTAAACATTACAAGCATATACAATTTCAAAATGCACAGATCTCCTTCCTGACAAAAAAGATCAGTACTATGACTTTGACTCT
CTTTTGCTTTTCTTTATTTAACTCTAAATTTTCCAAAGGTTAAAATATATACACCCACAGAAGGAAGACCAGTCCTGTCCCAGTTAATTAAAAT
GTTTGCTGGGTTGTTCACTGGATTCCTCTCATGGGCCCAGCTGGAATTCACTGAACCCTATATTGTAGTCATTATGCACAGCTTTTAGAAGGAA
AACAAGGCATGGCCTTTACGGGCGGGCACAGAGCCCACATAGCCTGATGCCTCAGTCTGTTGATGGGAAATGGCAGGACCTATTCGGGCTCATC
ACTGCATTGGAGTTCTCATCTCTTTGACTCTTAAAATAGATGAATATTTTTTTCCATTTATCTTTAAAGACATGTGAACTTGATGAGATCTTGC
TATTCTTTCATATTCTTGGAATGCTCAGAATGAAAAGGATCCTTAAGGTGACCTCCAGTTTTGTTTTCGATTATTCACACGGTTTATCTGACTT
GCCGTTACAATCTATGAGGTCAGTCTACTGACATCCTCTAGGTGTATATATTGTAACTTTCATATTATTGTTCTTCATTATCATGACATATCTG
AATATTGAATATGTATTTTTTCCTCTTCGAATTCACTGCATACATGTGTATCAGTTAAAAGAAACATTCTAAAAGATAAATGGGGCTGCTGTTT
TTTCTTTTTTTTCTGCTTCATTTTTCCTTCATAGGGGCTGCTTTTTGAGCACAGGCAAGGAGTCATTTGCCCCTTCTTCACTTTTTATAAGTTA
CCTGTCTCAGGACCTCTTTACATGATTCATTTCTTAGTTCTGCAGTGCTGCTGCTTCCTTCCGTAAGCCAGGTCGACGTGGGCTGGAGAGAAAA
TTACCATCATCGCCTTTCTAGGCAATAATATGCATACAGCTGTACACATCAAGTTGGTTTTTATCCTTTGGCCAGAAAGATAAAGCATCACATT
TTCATTTTTTAGAAAAAATATTTTGGGAGGTAGTTTCAGCAAATACTTCCAAAGATGAAAAGTCGTTTGGTTTCTGTAACAGAGGTCTCCATTT
CCCTTAACCACAGCCCTACCTATAAGGATGCTTTGGCAAAGAATTCACCAGGACTTTGCCTGAAGATCTGTTATTTTGTAACGTAATGTTTCTT
TATGTATTTCAAGACTTTGTGGAGCAAGGTGAAATGAAAGGTGAAGCAAGGGACTTTCCCTGCCTTTCCTGACCTCTATAGAACACCTTTGGAC
AGATGGAAAAGGGTAGTCCTTCATAACAGTGAATTTTCTCTGGTGTACTTGAGCTTAATTATTTTATACCAACCTTTTTTCAGTCTTTGGTATC
AAATGACATGAAGGAGGGAATACAGAAATAATTAAAGGTCTGTTATTTTCTTTATTTTTGTTCTTAATTATACAAGTAATATAGCTCATTATAG
AACATTTTTAAAAATGTAGAAAAGAATATAACTATAATAATGATCACTCATATCCAGTTACCTATTTTGGTGTATTTCCTTTGTCTTGTTTTCA
TGTCAATTTTTTTGAGATGAAAATATATATACGGTTTTATATCTTATAGTTTTCACTTAGCTTTATAGCATGAGAATCTTCTCTAGTCATTAAA
GACTTTTTGTAAACATTCTTTTTAATAGCCATGTAATATTCGAATATGATAAAGCATAATCTATGAATAGATTGTCTCATTTTGGACATTTGGT
TTATCTATAGTTCTCTCCCTTATAATTATCTCCAACCCACATGATTGTGCCTGAATCTTTGTCATGGCTCCTTTGATTATGCTATGCCTTATCC
TACCACAGCATTTGTGTGGAGGGACAGAAGATTTTCAGTGGATGGGTTTTCTTCCTTCCCTGCTCCCACCCCAGCTCCTTCAATATACTAATAG
ACGAATCAGACTTTGTCTTTTTCTGAGTTCTCTTTGTGGCAGTAAATTCCTTTTGGAACCCAACAAATTGTGAGTCCTTGGTATCAGTTTTGGA
TTGTGTTTTTTAACCCCCTCCTTGTAGGTATTGGATAACAGAATTCTGGGTCATGTTTAAAATGGACGCCAGCTTGACAGACACTATGGAGGAG
TTTCAGGAACTGGTGAAAGCTAAGGGTGAGGAGTTACATTGCCGCCTGATTGACACAACTCAAATGTGAGTGTCAAGGTCTTGACTCATCTTTC
GAAACCACCTCCCTCCTGTAATATAATCCCAGTTGCTTGTTGGGCTTCTCACCCCCAGCGGTGACTGCTGCTCTCTTTCTCCACTCTGAGAAGC
CCCTCAAATACACAGGTTCAAACTGCCTCACCAAAGTGAAGGCTTGGCATTTAACAGAAAAGCAGAGAGGCCTCATGGTGATAAAGCAGTAGGT
GACATATCCAGGAGCAGAAAAAGTAAGTCCCAGTCAAAAAGAAATCAGGTAGCAAGATATTGACTAAAATTAATCCAGATAATGAAAAACTGAA
AGTGCAAATGACTGAACCCACGCTTAATGCCAGGAGGCACACCTGAACTTGTAAATGGGACAGGACTAAAAAATGTATTCAGGAGAAGGGTTAT
AACATATATGGATGTCTATGAAAGACACTTAGACTTCCAGAGCTGGATGGGATTCCAGGAATGTTTTAAATTATTCCTTGGAATTCTTATTTAT
AAGACTGACCCATTCTGGGAAAATGAGTGCCTTTCCTAATTAGGGGGAGGGGAATCTATGCTTTGTATGCCAGGGTTTTCACAGTGTGGCTGCT
TTATTTTGAAATAGTACAAATCCCTAAGAAATAGAATTCTAGAACTGATATATATTCTAGAGATCATTTCATCCAACATCCTGATTTTTCTGAC
TGGGAAATTGAGGCTCAGCAAGAATGTGACTCATCTAGGGTCCAGCTAGTTAAGCTAGTGACAGACCAGCAGCTAGCCTCTGAGGTCCTGTCCA
TGATGCACTGTAGCCACGTAGAAATGATTTCAAGATTACATTCCAGAGCCCCAAATAAGTCTGTTTATCAGTTTGGATTCTCTGTACTTCTCTG
CTTAAAATTACACAACTTGAAAGATAAGAGATTATTCAAAGTAATAGTTTCTTCATTTTAAAGATGAAATTAGAGCCCAGGGCAATTCTCAACC
CCAGCTCTCCTTGTCCCAGTATTTCCTAGCTGACTTTTCTTGACCAAAATTTTCCACATTATATTCTGTAGGAATTATTAGTCTGTGGGGATAT
TACTTGGCCAGATATAACTGAGGAATAATGAGTTAAACTTTCCTTTTGCAGTGCTTTTCAGAGTGTGTAATATGCTCACTGGCCAAAGAGGCTA
CAGAATTTATAGCACTTCCCAAGCTCATTGACCACAGAACCCTTTTATCAAGAGACAAGGTTCCCAGGAATACCAGTTTGGAACACAGGCAACC
TGCTGTCTTTCAAAATGGGGCCGCTTCCCTTTGCTTGTTTAGTGAAGGAAACTGAATAAGAAATGCATGGGTTGATTACAAGAAAATAATGGGG
GCAGGGCGCACTCTAGGGTCAGCTAGGCTTCCTTGGTCATTTCAGTAGCTGAGGACATTTGAGGAAAATGACACAACTCTGCCAAGAGTGACCA
TATGTTTCTACGCGTGATTCCCTCCCTTTATGACATCTTGCTGTGGTTCTTTGAGCTCTCTTTCTGGCATGAAAACTGTTTCACTGCCTTGGGA
TTTCCAACCAGATAGAGAGTCCCTATCATTCACACAAGAATGAAGGAACACACAAGCAGAATGGAGGGAGCAGACATACCAGATGATACACAAA
GGAGCCAGGACCAAAGGCAAGGAAACTGACCGATACCTGAGACTCCTTCCTTCCCACTGCCTTAAAGTAAACTCATTTATCCTGTACCCAACTG
CACACTTAAAAGGCTCACTGAAAACCAGCAAGACCTCCTGGAGCCTGCCATGGAAAAAAACTGATTTGGAGAAACTCTGTGAATGCACTGTTTT
GGCATTCACAAGGAAAGTTAGCTAATGGTGTTTTGTATATTCTTGTGAGAAATGTTAATGGATTTACTTGCAAAAAGCGATTTATTCCTTTTCC
CTGTCTTCTCCCTTTCACATTTACCCACAAAAGCAATGCCCGTGACTGGTCCAGGAAACTTACTCAAAGGATAAAATCAAATACCAGCAAGAAA
CGGAAAGTCTCCCTGCTCTTTGACCATCTGGAACCAGAAGAGCTATCCGAGCACCTCACCTACCTTGAGTTCAAGTCTTTCCGGAGCATATCGG
TATGTATGCAAGGGGCAGGAGAAAAATCTTCCCTGGGATATTTCTGAAAATAACATGATAATTCAACAGCCCATCCGCTTCATAACCTTAGAGG
CCCCCTCAAGTCCTGGCAGGCTTTCCTGCCAGACATCATAGAGTCTGAAAAAATCAGTACCATCTGAATTCCTTTGATTGTGACTCCTGTGCTT
CTCCCATCTCTCTTCTTCCATGCCCCCGCCCCATACCATCCCACCACTCACTCAAGTTACCATACTGTGGGCACACATCACACACACACACACA
CACACACTCTCTCTCTCTCTCTCTCTCTCTCTCTGTCTCTCTCATTCTACCCTCATACCCCTCATAATCCTCATACCCCTGATAATCATTAGGC
TTCAACAACTCTTTTTAAAATCTTCCTCAAAAAAAAAAAGGGGGGGGGGTGCGGGGGCGGGGAGAGGATATGTCCTCTCACAGTTTGAATTCAT
TCTGGGGTAAGCTTTGAATGGCTCTGGGAAATCCCAAAATAGATTCTACTGCCCTAGCCCAGAACATACCTAAATCCTTGAAGTGGTCCTGGCC
CAGAAGTCACTGTGATTCTAGAGATAAGCTGTGCTGACCCAAAACCTTTCAACTGGGGCAACTAAGCAGAGTCAAAGGGAGAGAGATTTCAGTT
GATCATCAGGTACAGAAAGAGTACAGGTTCATCCTTTCCTCACTGCAATCCATATGTGATCCCAAAGAATATTAGTTCTATGCAAGAGAACTTG
TCAACATGTCAGATGCCAACAGACCCTCACTGGCTAATGTGCTTTCCTGAGAAATGAAACCCTTATTCCACTTAGCCCCTACCCCTACCAACAG
ATACAGTCTTCACCTCCGTGCTAAAAATAGTGACCACCTAGTTGCTGGGTCCATTTATTAAAATGACCAGTATTCTCCTGGTGAGGGGATGGAG
GCCCAGAGGGCTGACTTGAACAAGGTCGACACAGCTGATTACAGTCAAAGCTGGGGCTAGGACTACATGCCTCTCTCAGTCACTCAGCACCAAT
TTGTTGAGCTCAGTTGTGTCTGGAAGGAGACCTGCAAATCAGGTTACAAAGGTTATTTTACATTTTCATTCATAATATTGACAGCCCTTCTGTT
TTCACGCAGCTGTTTGGTCACTCAGCCAGGAAGCTCCCAGCTTCCATTTGGAAATAACTTCTCCAAGCATTAACTCTCCATTCTCTGCACCCCT
CAGTACCCCAGACCCAAATTGAAAAGCTCTGCCTCAGGAGTTCCCATTTGGCAGTGCCTCCCCGTCACAGGGAAGGACAGCAGTCAGAGCTGAG
CCACTGAAAAAAGCCATTTATCACACAGGAAATCTGAGCTCTGCAGGGGTCAAACTCGCAGGCTGCTTGGTCAGGCCTGCAGGATGTCTCCTTG
ATGCCAATGCTCACATCAGTGGAGGCAGAAGTGAAAAATCAGTGCAGAACGAGGAATCCCAGCCAGTGTCCCCAGGGGTTGAACAGTGTTCTAT
TTGCAAGTGAAAACATCAGTTTCAACCTTCATTTCTTTTGAGTGGCTTGGTCCCTTCATAGAAGACTATCTCAGGGTTTTTTTGTTTTTCCTGT
ACAGTGCTAAACAGTTTTACCAAACACAAGTTTTCTCTCCAACATGAGGTTGGAGGGAAATTTATCTGTGCTGTGAATTGAAAGGCATTTGATA
TCTTCTGTTTATTACAAAACTTTAGGGGTAGTGAGGATGTTACTGGGATAATTGTTTTAAAAAAACAAGTGGATTTAAAGTTATATTTATAGTG
GGAGGGGGGTATTAAATGTATTCACTGGAATATATTAATTGAATGAATATATTCAAAATATATGCAATGAGTTGTGGCTACTTTCTTTTAGAAC
CAGTAAGATGTTTGCTTTATATATCTCTCTTAAGGAGCGATTTTGGTCCTACTCATAGGTTTTCCACCCATCAGCAGCCATATAGCACAGTAAA
ATGGGGCTCAGTTAATTTGCAGCTATTTTATTTGGGTTGTGACTTCTCATCTATATATTTACTACTTAGGCAGAACTAAGGTGTACTGTAGTAA
TAACCCCCCACCCACCCACCTCCTGTACAATCTCAGTGGCTTAATTAAGCAAAAGTTTATTTTTCACTCACACTGCATGTCTAACATGGGTCAG
CAGAGGCTGATCAGAGTTCCCCAGGAACCCAGATGATGGAGGCTTCATCTTGCCAGGAATTTCCAAGATCTCTGCAGCAGTAGGAAGGCAACAT
GGCAGACCACTCACTGGCTCTTACATCTTTGCCTCCAAAGTTACATCGCTTCCACTCAACATTCTGCTGGCTGAACCATGTCATATGGTCACAC
CTTCTTTCAAAGGGAAGAGTTGAGAAGGGAAGAGTTGGAAATAATTGGGGAAAAGCACCAAAGACTTCCATAGGAAACTTGAAGAGAAACAGAC
TGTGATTTGAGCAGAGGGATTCTTTGTGACAGGGACACAATAGCTACAAACATCACTTAGACTAGATCTTCAGGGGACATTTCCAGTTTTACAC
ATGTTGTTTCATTGCCAACTGTAGTCAATGCTATTGTCAACTTGGGAAACCATGATCAGCAAACATATAGCTAACCAAATTTTTAAGTACTCCA
TTTGCCTCTGCTCCAGTTGGTGAGTAGAAGGAAATACAAACAGGCTTGTATTAGCATAAGGTTCATAACTACTCCATTAGCGCAACCTTACATA
TATGTAACACTTTAAAAAGTTAAAGCTTTCACACATTATCTTCTTTGATCTTCATAACAATTCTATGTCCTAGATAGCCCAGATTTTATGATCA
CATTTTTAAGGTAAGAAAACAGCTGGGGAGAGAGAGATTTGTTTTTGTCAGGGGAATTACCTTTTATTCCCTTAACCTTAGCAGCAATACTGAT
CTTTATTCTGTACTGTATTCTTTAGAATTTAGAATAGTCAGTAACAATATCTACTGAAATACAGCATTTGGATTAACTAATGTGTCTACACATC
CACTTATTCCTAATAACTTTCGATGTAGAGAAATCATGCTGTGGGTAGAATAAGGAAGATTTCCCTCCCATCATGCTTGTTGGCAAGTGGCCAA
GATGCAGAAGAGCACCAGGAGCTCCTCTGTTCCATGGCACTTCCCAGCCATCAACTGAGCAGACTGGACTTGTCCCTTAAATCTTCTGGAATAC
CAGAGTACAGTACTGATAGGTTTTTTTTGTTGTTGTTGTTTCTTTTCCTGCAGTTCTCTGATTATCAGAATTACCTTGTAAATAGCTGTGTGAA
GGAAAACCCCACCATGGAGCGATCTATTGCTCTGTGCAACGGCATCTCCCAGTGGGTACAACTGATGGTTCTCAGCCGCCCCACGCCGCAGCTC
CGAGCAGAAGTCTTCATCAAGTTCATCCAGGTGGCTCAGGTGAATAACTGGTTTAAGACATTTGGCTTGGGCTATAAGGTAAACTTTCTATCCC
CTTTTCCTCCAGCTTAGTCTATTAATTAGAGCTGTTTTTTCCCTCCTGAACCATGGCGAGGGGGTGGAGAGGTTTGTGTGCCTTCAACCAGATC
TAGATATGGTGACTTGTCTCTTCTTCCATACAAGGACAGTGTGGAGGGGAGGGGGCCATACAGGCCATATCAGGGGTCAGGATCCAGTTTTAGA
TCTGTTTAGACCTTTAACATTTTTCAAAAGATGCTTACCAGCCTACATTATTTTCCCGAGTCTGGCCCACATGGCAAAGGAAAGGGCAATTCTA
AGATTTACTGGAGCTGGACCATCCTGGATTGTCTTAGGTCTTTAAGCCCATGCAGTGCTGAATGGCTGCATTTAATGTTTGAGGGCCATTTTAT
AAGAGGAAAACAAGTACTATGAACCCATATTTGAAGTTCCCCAGAGATTCTAGCTTCTCTGTCCCAGTTACTGTCCTATATAGGCACTGGCATT
GCATGGGAAAATAAGACAAAGTCCTTACCCTCAAAAATAAAAGTCTGGTAAATTTGGAATAAATAAAAGATTACACTTAATAATATCTTTCTCC
AACTCTGGATGGTCTAGACCAGTGTTTCTCAAAGTCAACAGCTCTGACTTTGGATCACCTGCTCTGATATTAACTAAAGGTATATTAAAAGTAC
AGATCTCCAAGCCTAACCCCAGATCCAGGCAATCAGAATGTCTGGGTTAGGTCCAGAAACCATTGTGTTAAACAAATACTTGAGGTGATTCTAA
GAAAGCACTGATCTAGAAGGTGGTCCACAGATCAGCAACAACAAAAGGAGAGAGATGTAATGCCTCCCAGATGTGGAATCCACCAGTCTGAATA
AAGCAGAAGGGGAGACATGGACAGGAAATTGCAATGCCTCGTAACTAGTCTTTAACCAGCGTTAAGGGAGACTCACGGCACAGAGACAAGAGGC
AGCCTCTAAGCTCCCCATATAACTGACATGTACAGTGACTCTGCTGTCATCTGTCTTCTGTGTCACAGAAGCTCCACCAACTACAGAACTTCAA
TACACTGATGGCTGTGATAGGTGGGGCGTGTCACAGCTCAATCTCGAGGCTCAAGGAGACAAGTTCGCATGTCCCACATGAAATCAATAAGGTC
AGTGCTACTGTCTTCTCTGGGGCCCTTAGCAGCATTCTCAAGATGTGTGCCAACATTTTCTCCTTGGTGGGCAGCCCAGAGAGCATTTCCAACC
ACACCATCCCTTGTCAACCAGGCTAAGTGGCAGGGAGGCAAACCTTAAGGAAATCCATAGGATGGAGAAGTCACATTTTTTAGAGTAACTGTGA
TTTTGTCTGGCCTCAGACAGTGTCACTGCAAACATTCCTGCTGGACCAGTTGAAACAATGTGTGCCTCTTCCCATGTTCATAGGCCATGGAATA
TTCAGTTACCATCTTTGACACTGATTCCCTCGCTCCAGCTCCTTGCTCTAAAATGTGTTCTCAAAAGGAATTGTGGTTCGGTTGACCATAGGTC
AGTTTGCACATTGAGTGTAGTTATATAGTGATATATTAAAAACTTAGCTATCCAGAACCCTTGAAGAATGACATTCCGGATAGTTGAATTTTAC
CAGATAATTGGATATGAGTTCTTCAGATACCATTTTGTAATACATAATGTTTTATTAATGAATATTTTTCTTAAATGTCCTGTACATTTTCCTA
CATTATTAGATTTTACAGAATATACTTTTATCTTTTATGACATAGGGCTGTTTTGCTTGTTTCTCTTTTTTATAGAAACAGGGTCTTCCTCTGT
CATCAAGGCTGGAGCACAGGCTGGCACGATCATAGCTCACTGCAGTCTCAAACTCCTGGGCTCAAGCAATCCTCCCACCTCAGCCTACCAAGTA
GCTAGGACCATGGGTGCACACCATTATGCCCAGCTATTTTTTAAAATTTTTGTAGAGATAAGGGTCTCCCTATGTTGTCCAGACTGGTCTCCAA
CTCTTGGCCTCAAGCGATCCTCCCATCTCGGCCTCCTAAAGTATTGGAATTACAGGCATGAGCCACTGAGTCCAGCTGGAGCTGTTGTTATAAA
TATAAACCACTGTAATATACCGAGGATATAAATGTATGGCATGGGTTAGATAATGACTGCTCTTTGAGCACTTTCATGTAAGAGGACCTTACTA
TACTCAAGCCCCGATCCTTCCAAACTAGCATGGAGAGGAACTCTCTTTTTAGGGAGAGATACCTAAGCTCATTACCCCATCTGTTTTTCCCAGG
TGTTCAGCTCCCTTTTGGGGAGTCTTTTATTATCTCTGTTATCACAATGCTGGGCCTCTGGGGAGGCCCCCCACTTCCCAGTGGTCTGATCAAA
AGGGCATCATCTTTAGTCACCTCTTCATTCCTTAACGGTTCTGCTGAAACCAGGTGGAACAGTTGAAAAGATTGCCCACATCTCAGAGGTGACA
GAGAGAAATAGTCACAGAGATTAAGAAGCCTTCATGTCATTTAGCTTATAAGAGAGAGCTTGCTCCTCTTCAGCTTGGGGTCCCCCCACAGCAG
TGCTTCCTGTCGGTAGCTACTTGACCCTCAGGGTGGTCTGCATAGTGAATTAGTAAGTCCAGGGTTGAAGCAAAAGTCCCCAAGTTGTTGATTT
CACCTTCGGAGGGGAATAAAAATATCTTTATCATTTTTGCTGTTACACTCTTGTCTTGTGAGCCCCTTCCATGCCTAGGGCTCACCTGCACCAT
TGGTAGAGGTTTATGAAGAAGAAGCTCTCTTGCCATCTCCCAGTGATTTGAAGGGGTCTGACTCCAAGAACTCCAAGTTCTTCATTGTGGAGAA
ATGGTACTTCGTAGGCCACAATTTTATTAAGTTTGGGCACAGTGTCTAAAAGGGTTACAGTAGTCCCCCTTATCTGAGGTTTTGTCTCCTGTAG
TTTCAGTTATCTGAGGTCAACTGCAGTCCAAAAAAAATTTAATGGAAAATCCCAGAAATAATTCATAAATTTTATTATTTATTTATTGATTGAG
ACAGTCTCACTCTGTCACCCAGGCTGGAGTGCAGTGGCTCAGTCTCAGCTCACTGGAATCTCCGCCTCCCAGGTTCAAGTAATCCTCCTGCCTC
AGGCTCCCAAGTAGCTGGGACTACAGGCGTGTGCCACCACGCCGGGTTAATTTTGTATTTTTAGTAGAGACGGGGTTTCACCATGTTAGCCAGG
CTGGTCTTGAACTTTGGACCTCAGGTGATCCGTCCACCTCGGTCTCCCAAAGTGCTGGGATTACAGGCATGAACCACCACGCCTGGCCAATTCA
TAAGTTTTAAAATGCATGCTGTGCTGAGTAGCATGATGAAATCTTAAGCCATCCCATTCCATTCTTTCTGGGACTCCTTCATATCAGTCACTAA
GTAGCCGTCTTGGTCATCAGATTGACTGTCATGGTATTGCAGTGCTTGTGTTCAAGTAACTCTCACTTTGTTTAATAATGGCCTCACAGTGCAA
GAGTAATGATGTTGGCAATTTGGATATGCCAAAGAGCAGCCCTAAAGTGTTTCCTCTAAGTTCTTTACTTAATAAGGAAGGAAAAAAATCATAT
GCTGAGGTTGCTAAGATCTAAGTAAGAAGGAATTTCATCTATGAAATTGTGAAAAAGAAAAGAAGTTCATTCTAGTTTCTCTTTTGTACTTCAT
ACTGCAAAAAGTTATGGCCACAGTGCATGATGGGAATGGCATTACATTTGTGGGTGGGAAACATGAACAGAAATGTGTTCCACTTGACAGCAAT
CGGGTTTGTTACTATGCACAGTTTCAGGCATCAATTAGGCATCTTGGACTGTATCCCCCTAAGGATAAGAGAGGACTCCTTATGTTGTATGATA
ACATGATTGGCAGACAGTCACTTCATGTCTTTGCTTCTGAGAAAGCTGGTTATCACTCATGGGGAATGGATTCTGTGAACTGCTTGCTGACTTG
AATGACAAGTGATTGAAAAGAAAACTAGATCTGGCTGCTCCCTTTCTGTGCTGGTGAGCAGCCGTTCCCACTTCCAGGTTTGAAGCAGGGTGTA
ACCTCTAATGTCCTCTCTCCTGTCCTTTGCTCAGAATTGATCTAGTCTATTCAGTCTTTTTAGTTCAAGGTTTTAGTCTGTGAATGGAATGTCA
CTTGCTTTCTAGTCATAGGCCCATTCTGAGAATCTTGAAAGGCCCATCAAGCACCTCATAGAGAATAACACACAATAGACTACTGAAGCTCCAC
TCTTAAAATACATGCTCTTCCCCATGCCAGCAACATAGTCAGCATTTCCTGACATCACATTTCAACTGTTAATCAATGCAGTGTCTCTGGAAGC
ATTCAGAAGACACCAAGGAAGGGAATCACATGGCTTGGGGACAGGAGTTAGAGGGATAGTGTTCACTATATATTCTTGTACCTTTTTAATTTTT
GTATCAGGTCCTTGTTTTTGAAAAGAAGGAAATTTTAACATAAAAATGATAAAACCCCGATTTATAATGTTTAAGTTATATAACAGATTCAAGC
ATATCTTGTGTTTATAAATATATTCTGTGATATTTGACATAGCTGAGGTTTTAACTGGGGAGTCATAATCTGTACCTTTTACATGAATTACCCC
CTGCTCAACAAATTCTGGATGGGGTGAGATCATGGCACAGGATGCAAACATGCAAGGTCCTCATTACAGACAATGCACACTGCCTAGCGGGTAC
CTGTGATTGGTCTGTTCTGCTTTGTAGGTTCTCGGTGAGATGACTGAGCTGCTGTCCTCCTCCAGAAACTACGACAATTACCGGCGAGCCTATG
GAGACTGCACCGACTTCAAGATCCCCATTCTGGGTGTGCATCTCAAGGACCTCATCTCCCTGTATGAAGCCATGCCTGACTATCTGGAGGACGG
GAAAGTGAACGTCCATAAGCTACTGGCCCTATACAATCATATCAGTGAATTGGTCCAGCTGCAAGAGGTGGCCCCACCCTTGGAGGCTAACAAG
GACTTGGTACACTTGCTGACGGTGAGGCTCACACTGGAGGACAATTACTAAGCACTTTCTGTGTGCCAGGTGCCATTCTATACCCCTTACCAAA
TATTAGCTGTTAGTCCTCATAAAGGCTCTATGAAGCAGATCCTATTACTATCTCCAACATATAGCTAGGGATACTAAAGCACAAAGATATACCT
TGCCAGGAGACCACACATTATTAAGTAGCAGAGCCTTGATTCAGACCCAGGTGGTGGGACTCTAGAGACTGTGCCCTTCAGACTTCTAGGACTG
TTTCACTACCTCTACAAAAGCAGTCCTAAGCTCTCTGGGAGGAGCTGATGGCAAATCAGGATACAAGTCTTGGTAACAGATGGAGAGAATGAAT
GTGCTCTCATAAAAAGGGCAGGCAGTAATTTAAAAAACAAACAAAAAACACCCAGGGTTATTATTTGGTTCCTGGCTTAGATCTTTGCTCTTGG
AGATATTCAAGTGAAGATGAACACTTGTCAATGGTGTGGGAGACCAGGATTTGCATTTTGCATATGGGGTGAGATCAGATGATGAACTCCCTTC
CAGACCTGCAATTCCGTAGTTTACCAGCTGTGTGACTATGGACATATAACCGTCCAAGATTCCTCTCCTCAAACGAATTAGNNNNNNNNNNNNN
NNNNNNNCCAGTTCTAAAGTTACACAGTTTTTAACCTGCTGTTTTTGGAGAGTAGAATAATGATCATAACCTCTTTCTGCCTCACAGCAGCATT
TTGATAGAACCCTGGGCTTCACATCAGCTGGTGTGTGATCCTAGCCCTTTCACTTATTAGCCAGGTGACCTTGGCAGGTCTGTGTCTCAGACAT
CATTTGTAAAAACATGGAGTGAATCTTAACATTCTTTGGATTATAAATCTTGTGATGAAAGCTGTGAACCTTCTTCCCAGAAAAATGTACATAA
GCCATTTATTTTTAAATATGCATGTAATTTCAAGTTCTGAAATCCTTGAAGCCTATCCATAGACTCAAGGTTAAGAATAAGATTATTCCTAAGG
ACTTCTCTCCTAAAATTTTATAGTTCTAAGATACATGAAACTCACTATTTTTTTTTTTTTTTTTTTTTTTGGAGACAAGGTCTGGCTCTATGAC
CTAGACTGGAGTGCAGTAGTGCAATCTCAGCTCACTGCAACCTCTATCTTCCAGGTCCAAGCCATCCTCCCACCTCAGCCTCCTGAGTAGCTGG
GACCACGGGCACACGCCACCACGCCCGGTTAACTTTTGTATTTTTTGTAGAGACAGGGTTTTGCTATGTTGCCCAGGCTGGTCTCAAACTCCTG
AGCTCAAGCAATCAACCTGCCCTGGCCTCCCAAAATGCTGGGAATACAGGCATGAGCCACCATGCACCCAGACTCATTAACTCTTAATAGCAAT
TAATATGTCTTACCATTTAAACTGGATTTTATTGCTGGACTCAAATTTAATAGTCTCAGACTATACACACCATCCTTTCAGATCATCTGTCTGC
TTTGAGAGAGAACAAGTGATGTATTAGTCATCTGTCCAGACAAGGCCTTTGGCCTAAGCTCTATATAGAGGCTTAGATTTAAATCATAGTTAAA
AAGAGGGCATCCTGAGATATGTCCAAAATTAAAAACATTTCAATGAGAATTAATGCTTGCCCTATTGCAATAGTAGGTGTTATACCTGTTTTGT
GACTTTTGAAAACAAACTGTTTAGAACCTAAAAGAGTAAACTACTGGCAAGGAAAATCCAAGAAAGAACCATCAGCTGTGGGAGAGGCAAACAG
CATCTTAGCAATGAACCCTGTTCTGCTTTGATCTGCAGTTATCCCTGGATCTTTACTACACTGAGGATGAAATCTATGAGCTTTCCTATGCCCG
GGAACCAAGGAACCACAGAGCTCCAGTGAGTTTTTCATATTAAGACTCCACATGTAACAGGTGCTTTACAAGCTTCTCCTCGAGTTCATTCATC
CTCAGGAACAGACTTTCAGTTCAGTTTAATACATGTACTGTTTTTCTTAGTGCTCAACCTTATGCTAGGAACAGAGAAAGCTTTTCAACATATG
TCTGAGGAATAGTTCCTGTCCATTAAAAAGCTGTGATATAGTTGAAGGGATAAGCCTAATGTGTGACCAGTGAATAACTAGTGAACAGTAAATA
ACTATATCATCAGAATGCCAGGTGTGGTGGAATCAGTCAGTGGTTTACAAAGTAGAGAAGGAAGATACTATTGTGCACCAGAGTGACATCCCCC
CCGCATTAAGAAGAGTCCCCACATAAATTTCTTTTCTATCATTCATTGATGATAATGAGAGTAATATGCGTACTAATCAGAAGCTAAATTTCTG
AAGATTTCCAGGTTGGTAGATAGAAAAGCCCCATTTTCCTGTAATGTTCTGGGTGAAAAGAACAGTCTGCTGAGTCTTTTGACCTCCTATGCTG
AGAAGTCCTCAAAATAGTACACAGTCCTGGCACTGCCTGCAGTAGGAACTTCACAGGTTATCTGCCCTACCCTCCCACAGGCAAGTAAAATCAC
TTCAGTCTCCTGGGACGTTTCCAAACCCCAAAACAAGTGATACTCAAGGGAAGAGAGTTGCCCACACTTAAGAAATCCAGCTCATCTATTGGGG
TGGCTAAATTGCACTCCTCCACACAGAAAGATCAAGTTCTAGCTTTCTATTGTAATGAAAGATTCCACTGAAAATATTTGGTAGATTCCATTTT
CAGGTTAGAGGCATTTCAATGGAAAGGGTACATTCTGTTTTTTCAGAGAGATTTTAATTTGGGCTGAGTTTCAAATACAGTCATGCACCACATA
ATGACATTTTGGTCAGTGATAGACCTCATATACCACAGTGGTCCCTTAAGATTATAATGGAGCAGAAAAATTACTACTGCCTAGTGACAGAACA
ATTGTAACATTGTAGCACAACACATTACTCACTATGTCTGTGATTATACTGGTGTAAACAAACCTACTGAGCTGTAAAAAGTCTAGGTTATACA
CTTACGTACAGTACATAATACTTGATAGTAAGTATTACCGGTTTATGCACTTACTATACAATACTTTTTATCATTATTTTAGAGTGCATTCCTT
CTACTTAGAAAAATTAACTGTAAAACAGCCTCAGGCAAGTCCTCCAGAAGGTGACAGCTCCATGCATGTTATTGCCCCTAAAGACCTTCCAATA
GGACAAGATGTGGAGTGGAAGACAGTGATATTGATGATCTTGACTCTATATAGACATAGGCCAATGAGTGTAATTGTACCTTAGTTTATAACAA
AGATTTAAAAAATTAAACATTTTTAAAATAGAAAAAAGTTTATAGAATATGGCTATAAAGGAAATAACTGGTACAGCTATAAGCTATACAATGT
GTGCTTTAAGCTAAGTGTCATAACAAAAGAGTAAAAGTTAAAAAAATTAAAAGCTTTTACAGTAAAATAAGGTAATTTATCATTGAAGAAAAAA
TTTAAGAAACTTAGTGTAGTCTAAGTGTACAGTATTTATAAAGTATATAGGAGTATACAGTAATGTTAGGCCTCCACATTCACTCACCACTCAC
GACTTGCCACAGTAATTTCTAATCTTGAAAGCTCTATTCATGGTTAAGTGCCCTATACAGGTGTAACATTAAAAAAAAATTTCTATACCATATT
TTTACCTTTTCTATGTTTAGATATGTTAAGATACACAAATACCATCATTACAATTGCCTACAGTACTGAGTATAATAACCTGCTGTTATAGGTA
TGTAGCCTAGGAGCAGTAGGCTATACCAAATAGCCTAGGTGTGTAGCAGGCTATACCATCTAGGTTTGTGTAAGTACACACTGTGGTGCTCATA
CAATGACAAAATCACCTCATGACACATTTCTCAGAACATATGCCCAGTCATTAAGTGATGCATGACTGTATTTAGAAAAGAGCTTGTCCAGGCT
TCCGATGATTTTTCTTGTGCCTTCCTGCCTTTTAGCTAAGCTTTTCCTTATTCACATTTCTACTGCAAAATGTGACTGGAGAATAGAGAGGAAA
CATAAATGATCCACTTTACTCTTTGTTGATAGCTCTGACAAAGGGTTTAATGGAGAGGGAATTGCAACTCAGGCAGATTGAGAATTTCAAATTA
TCTGTAGCTGCAGATAATGTATGTGAAGAGCACTCTGGAAACCGTAAGCACAATGCCTTGTTAGCCAAAATTATTAGTAGAGATTTCAATTGCC
TGTGGGTTCTCTGCTCCTTGCCACTGAACATAATTGAAGATTGTCCATAAATGTAACCGTCAGGTAGTGGGAAAAGAAAGAGCTATAGGTTATA
ATATTGCATTGTAGTCATGAGGATTTCTCTAAATATTTCATTTTAGCCACTAACACCTTCAAAGCCACCAGTAGTAGTGGACTGGGCTTCTGGA
GTGTCTCCCAAACCTGATCCAAAAACCATTAGCAAACACGTCCAGAGGATGGTGGATGTAAGTACAGCTGTGTTCAGCACACAAAAACTAGGCA
TTGCCTGGCTCAGTTTTGTATGTGGGGTAAAGGGAGAGGAAAGCACAATGGTGGAAATGTAAAGGCACGAAACTTGAACCACTTACTTCTCTTA
CTGTGGCTTTAGAACAATTCAAAAGTTACATTTTGACAACACACACTGGACTCATATGAAGGCAGTTGTGCCTCCTCTTCAAGACCTAGCAAAA
GAGCATATGCTTAAAGGTTGTCAGTTGATTTAGGAGTACAGAAGTATTGGGATGCATTTGAGTATAGTAAGGGGTAATCAGGTAAACCTAAAAC
TGAAAACCTTTGTACTGCTTAATTCAATTCTCTGTGCTAATTGCTGAGGAAGGGGTATATATTCTCATACTTATGCATGTATCCTCGTGGACAA
CTCACATGTAACAATGGAAAACTAAGAGCGTAACGGTGAGAAACAAGAGGATTAACAGTGGGGACAGAGCACTTCCAACTGGGCTGTTTGTAGA
AGCTTCTCAAAGAAGGTACAACTGGAGCTGAGTGATGAAAAAAAGGGTAGAGTGAAAGGAGAACATCCCTCTGCGCAGTGAACTAGGAGTAGCC
AGCGTGATAGTATAATTGACGTGCTAATGATTCAAAGTACATGGCCACCCCCCACATGCTGCTACCAGCAAAGGCCTGAGATGTGACCTGCCAC
ATCTTTGCCACAGCTAAAGGAGCAGGTTTCCCTGTTCCTCTCGTTCAGGTTGTTAAAAGGCTTTTATCCAACTGCCCCAGCTCTTTGTCATTTG
ACTAAAGGGAGAGAAGACCGGTTTCTCTTTAGTAATCACCAAAAAAACTCCTTTTTCACAGTCTGTCTTCAAGAACTATGATCACGACCAGGAT
GGATACATTTCTCAGGAAGAATTTGAAAAGATTGCTGCGAGTTTTCCATTTTCCTTCTGTGTGATGGACAAAGACAGGTGAGGGTTTGTACTTA
GGAATATGTGGTTATGAGCCCATTGGTGAGAATAGGTTTATAGGTATGTCACTAGAAGAATTTGAAAACTCAGATATGTTGCTGCTGTGATCAC
TTAAAGAACAGACCATGAAATATGCATTCTTTTAAAAATATTCTGGGCCTGGGCACAGTGGCTCACGCCTGTAATCCCAACACTTTGGGAGGCC
GAGGTGGGCAGATCACCTGAGCTCAGGAGTTCGAGACCAACCTGGCCAACATGGTGAAACCCTGTCTCTACTAAAAATACAAAAATTAGCCAGG
TGTGGTGGTGGGCACCTGTAATCCCAGCTACTCGGGAGGCTGAGGCAGGAGAGTTGTTTGAACCCAGGAGGCGGAGGTTGCAGTGAGCCGAGAT
CATGCCACTGCACTCCAGCCTGGGCCACAAGAGCAAAAACCTGTCTCAAAAAAAAAAAAAACAAAAACTGAATTGAACATTCCATAAATCTGCC
ACTTTATAAATAGTCAACTATAAATAAGAAAAAGTTTAGTCTTCCTTCTTGCTTTGTCATTACAGATTTTGGGAAACAACTAATACTTATTCAT
ACTTTGCATTTTTACTTTTCTCAAGGTTAAATAAATTCTATCTTGGTTCTCTATAGGAAACTAATCATATGGTTAGTGGGTTCCAGTTAGGTAC
TGTCTAAAGTCAGAAACTGTAGCATAAGACATTTCCCTCCCTATACACATGTACATGTTACATTGTTTCTTTTTCAAGTCTTATAGATAGTGGT
CAAGTTAGTTAGGGAAACCTAGTTTGGCCAAAGAACACCTCTGTGGAAATGAGTAATGCTGCCTTACCTACTTACCACATGTCATCTTGTATCC
TTGTTAAGAGTAGGGCCCATGGAAGTAATCATGAGGTAGCACATACCATATTACCACTGAGGATAAAAAGAAAGGTGAGTTTCTAAGGCTAGAA
GCCCCCCCTTGCTACCTGCCTCCATGCCCCTTATACTCACATCCTTGCCTTGGTCATCTGCCGGCAGGGAAGGCCTCATCAGCAGGGATGAGAT
CACAGCCTACTTCATGAGAGCCAGCTCAATCTATTCCAAGCTGGGCCTGGGCTTTCCTCACAACTTCCAAGAGACCACCTACCTGAAGCCCACT
TTTTGTGACAACTGTGCTGGATTTGTAAGTTGTTCTTAGGGTGCTCCAGGGCTGAATTTGGGGAAGAGTGTGGGATACTTTAAGACAGGCACAG
ACCTCACAATCCTTATGAACCCATCCCAGCTTGGAGCCCACACAGACACCTAGAATAACCTACAGCTCAAGCGTGTGGTTTTTACCTGCACTCA
TCATCTCTCGCTTCTTCCTGTTTTTTCTTTTCTATTGCAATGAGGCCTTAGATAAATGGTACCAGAAATGCTGAATTAGGATATACTTTTAAAG
TATAATATTCATGTGCTGTTTCAAGTGTTATAACAAAGACATATGAGATACCTTCTGTCTTTAGGAGATAACCTTGGCTATGCCCCTCTTCCGT
TCCACTCCTGACCAATCGTTTCCCCCTAAACTCGGGTCCTGATGCAAGGCTCTATATCCTTCTCAGTTTGGAGTCCTGAGGAACCTGTTTGCCC
GATGATTCATCCATTTCTCGAGAATGTCTGGGTCATAAGGCTGCTAGTGAGAGCCAGGTGTGGTGGCTCATGCCTGTAATCCCAGCACTTTGGG
AGGCCAAGGCAGGCAGATCACTTGAGGCCCCGAGTTCGAGACCAGCCCACCTAACATGGTGAAAACCCGTCTCTACTAAAAATACAAAAAAATT
TAGCCAGGTGTGGTGGAACATGCCTGGTAGTCCCAGCTACTTGGGAAACTGAGACAGGAGAATTGCTTGAACCCAGGAGGTGGAGGTTGCAGTG
AGCCGAGATCGCACCACTGCACTCCAGCCTGGGCAACTGGGCAAGACTGTCTCAAAAAAAAAAAAAAAAAAAAAACCTGCTTCTTCAGAAACTT
TGATCAGCATAAACTCTTCTCATGGATTTAAGGTCAGAAGGGACAAGACATGAGAGATAAATGAGGTTTCCTTCCCACTTGAGCTAGAGAGTAA
AATGAAGGAAAATAAAGGAATGTCACCTTGTAGCCTCACACCAGATGGATGAATGACATGTGGTGCATTCTGTCTCTTGTTTCATAGCTCTGGG
GAGTGATCAAACAAGGATATCGATGTAAAGGTAAGACTCAATATTGTTGACATATTCCTGGTGTATCAGTCCATTCTCACGCTGCTATAGAGAA
CTGCCTGAGACTGGGTAATTTATAAAGGCAAGAGGTTTAATTGACTCACAGTTCCACAGGGCTGGTGAGGCCTAAGGAAACTCACAATCATGGC
AGAAGGGGAAGCAAACACATCCTTCTTCACATGCTGGCAGGAAGGAGAAGTGCTGAGCAAAAGGGGGAAAAGCCCTTATGAAGCCAACAGATCT
CATGAGCAGTCACTCACTATCATGAGAACAACATGAGGGTAACCGCCCCCAGGATTCATTTACCTCCCATTGGGTCCCTCCCATGACACATGGG
GATTATGGGCACTACAAGGTGAGGTTTGGATGGGGACACAGAGCTAAACCATATCAGCTGGTCTTAGAGTCCCACCTCTAAAACATTTTTTACC
CATAGTAGTTGTCCATTTAGTCCACAACTTCTCAGCAGAGTTCAGAGTCTCATCCCCAAAGTGGTTTCCCTCGGACACCCCCGGTGAATGACCG
CCACACCGAAGAGAGAAATGCCTGGTTGCACAGATAGGCATTTTCTCTGTATAAAATCCTCTTTCTGTCGTGGCTGGCCTAGAACAGGGATAGC
AGGTAGGAGATGGTGCTCTCATATAGTCATGGAAGGGTGGGTCCCCAGTGAAAAAGATAGAATCTCTTCTGCAACAGAAGATAAATGATTACTA
AAGGCCCTTGTCAGCTTTCCTTTATCACTCAGGACATAACTGTCCTTCCTTGTGACTAGGAGAATTCAGGAGTGAAAGAGAGGAAACCTTTTAC
TGCAAGAAAGAAAATAAGACCTAACAGCTGAGTGTTTCCGGAGGGTCTCCCTAGAGTAGGCAGAATGTATGGGATACCAGGACACCATCTCTTC
CCTCCAAGATATCAGGATACTGCTTCTGATGCTGAGATTCACTGATAAGGGCTACATCAAGAGTGTGAGCTCTGGGCTTCACCAAGTGAGAAGA
ATGCTAGCTTCATTAGGAATGTTAAGTGTCTTAGCTCCAGAAGATAGAACAGACATGTGTGACAAGAAAGCACTGCATGAATTTGGTTGTGAGA
CACACTGGGGAAGAGAGAAAAGTGAAAGACTTCTTGACCCAGGATGTCTTGTCCACAGACTGCGGGATGAACTGTCACAAACAATGCAAAGATC
TGGTTGTGTTTGAGTGTAAGAAGCGAGCCAAGAACCCAGTAGCTCCCACAGAGAACAACACTTGCTGTGGGGCCAGTGTCCAACCTTTGCTCAT
TGGGAGCCAAANNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNACTTAGCAGCAGGAAGCAGACTTAGCCACAGACAGAGT
TCTACTAGCTGAGTGACTGTCTATGATAGCCCCAATTTTTCTCCAGGAGAGGAACCCCAAGCCATTAATTTAAGACCAGGTCCTAGTCTGAGGA
ACCCTGAGGGGCCAGGTAGCAGGTAATTGTCAAAATTCTAGAAACAGAAGGCTAAATGTAGAAAGGAAGGGCGGGGGGCAAACTCCCACTCAAG
TGGAGCTTGCAGATTAAAATTCCAAATCATACAAGGAAAATCCTAGGACTGAGACAAAACATTCAACATTCAAGAAATAAACTAGCAAATTCAC
TTACATTAAAATGCAAACAACAGTACAATAAGGAAATGACTTTGAGTATGTTTGAGATGCCCCAGATCTAAAGTGCAAAGAAGCAGAATAGACT
GTCTCTGTCTTCATGGAGTAGTTCTAGTTAGGGGGAAGATAATATACAAATATAGAGCAGTTTAGAAACAATGCAAGAGTATATGCTAAGAGCC
AAATAGGTTGCAGTGACAGCAAATGACAGGAGGACATAGCAGAGAGATCGCTGTACTGGGGCTGTTGGGACTTTATGGAGAAGGTGGAATTTCA
GCTAAGTCTTGAAGGAAGGGTAGAATTAAGTTATGGGAGGAGGACAGAGCACATTCCAGATAGGATGTACAGTAGGAGCAAAAGGCCAAGGGCA
GAACAGCTTTAGTACTATTGGGATAGTGGGAAAAAAAAAAAGGTGTTAAGGCGATTTAGGTAAGAGATGAAGTAGAAAAATGAGACAGTCTAGA
GAGTCTTAAATGACAGGTGCCTGTCTGTATGATAAAATGAGGAGCTATTTAAAAAAGGGTAACATGAAGGGCTTGTTGGGGACGGTTGGGGCAC
AAGGCTTCATAGTGCTATACTAGGCCCTGATCTCTGGGCTGGCTGGTTAGCCAGCATGTCAAAACACTGATCACCTTGACTAACTAAAGAACAT
ATAGGTCTTCAGCAAAAATCCAGTGCTTATTAGGTTAGTGCCAGTTTAATATAAGAAGCTGTGTGACTTCTAAGTAGAACCAAAAAGGGAAATA
AGAAATGAGTTTATTTATATAAAGCACTTAAAATCGGGCTTAACACGTATGTACCATATGTTAATATCATTACTCTTTTGAAATAAGCTCCCTA
AAGTGGTAAGGCCACTATCGTTACAACAGATGCCTAGAGAAAAAGTCCATATAGTATACATTCTGCATTTAAAGCACTTCTAATGTTCAGTGGT
AAATATCCACATGCATTTATTGATCAATTAGATCAAAGCTTAAGTGCTTCACTTCCATGTAAGAAATGCACATTCCTTTTTATCACATCAGCCA
AGTAAAAATTTTCTGTCTTCCTAAATCTTTCCCCTTACTATCTATCTAAACACAGGCTGACTTCAAACTTTTGGGGTGTTTTGTTCTTAGGGGG
AAAAAATCCTCTTAGGGTCTTTTCTAAGAAAGAATATGCTAATCAACTAAGAATTTGATGTTCAACTTTTAATAGAAATCACAAACACCATTGT
GAAAAATAGGCCTGTACCCTGTATTTCATTCATGCACTCTGTATTTTCAGAGTTACACATAACCTTAGGGCACCACAATTCTGTCACATAAAAT
GCAGATTTTCAAGTGGAAAAGACAGCAGAGAACAGCCTGAATCTGAGGCCTCAAAAAGGAATCCCTGAAGGGGCAGTTGTTAGAGATTTCTCCT
CTCCAGGCCATCTCTCCCCTTTTCCAAACCCTGCCCCACAGTGGTAAGATAAGTACTTGTCAGGAAGTAGACCAATAGGTAGGATAAACATACA
TGTATCATGGGCAACAAAATGAAAATTACTATATTATTATAAAGTTGCTAAAAGTCAAGGTCTATTTATAATGGCAAAAATGACTATTGGATAT
CATCCATTCTTAGTTATTTCTATCTCATTATTATCGGGGATGGTTTCTGCTTATCAAATCAAAATTTCCAGTGATTTCTTTTAGCCCATTACTA
TCTTTCTCAATCTCATCAACTTTCACTTTCTTGAAACTTGTACCTCTCCTAATTTCTGTTAACACTGTCATCTCTCAGGACTCTTCCTGCAATT
TTAAGGGCTTTCTGTTGCTTTTTGTTCTTACTGCTTTCTTCCTTTTGCTCCTCAAAGTGTATAGTGCCTGAATTTCTGTCCCAAGCTCCCTGTA
TGTCTAGGAATTCAACCTAAATAATGGAGGGGATTATATTCATAATTTGACAATCTAGGTTGGTAAAAAGAATGTATAACTGTATTACAGAAAA
GTTGGAAAATGGAGATATAAAAAATAATCACCCATAATACTCCTCCAAAAAAAAAATGTAATTATTTTAGGGTGACCATAACAATTTAAATATT
TGAGCACTTAAATAACTATGTAATTACAATTTTGAGCTTTATTGGATAAATATTAAATGTTAATTTAAATTATGTTCTCTTTGGCAAGAATTAA
ACTAAGATCTTTGTAAGATTTCTAGTTTTACAACTGGTTGATTCTGGTCAAATACTAATTAGAAAACTTTTTTGTTGTTTTTTAAAGCCATCTT
GACTATACTAATCCAATGTCTTCAAAGTCAGACTATCCAATGTTTTAATCACTGCTGAGTCTTTCCAAAAAGTAAACTGAGATGATCTTCAGTT
TATCTTAAAGCCTATCTATCGCCCACCTCAAATACATCTGCTCTTACTTTACTTTTCCAGGAAATTACTCCTTATGGCTAACATTACTAGGCTG
GATCCTGGGTAGTGGGTCTTAGTTAAAGAAAACCAGAGTAACTTACCTTTTTCCTCTCAGTATCAAACAGGGGCAATACATTTTAAGGTACATT
TCCACAAAGCTAAAACTTCTTTCTCATGGTTAAGCTGTGCCCACTGTACAAACGGGTTCCCAGAGAGTACTTACCTGCTAGAGGGTGTCTGTAT
CAGGACTGAGACATGAATGTGGAGAGCTCCCAAATTCCAGGGTCCAAAAAAGTATTTATGGAGCATTTCTGTCTAGATATTGTTCTTGTTCTCT
TTATTTTATGAAACTCTGTGGCACTGAGGCAGGGGCTTCAACTTACCTCCAAAAACAGAAACCCTCTAAGCAACCTTTGTGCAAACAGAAGATG
CTCTTTCTCAGGGTGGATGTAGCTTGACACCAGTGGCCATGTCTTGGCCAAAGCCCCTGGATTTAGCCCTCACCCCCTAAACTGGGTGCCTGAA
AATGGGTCCTCTTTGAACATATGGAACTCCAGTAACAATAAGAAAGGTCTCATTGAAACTAATGTCTCAGTCTTGAGTCTCTGCTTTCCTGAGC
TAGAGTACTAGCATGTCCTGTATCTTCCTACAGCACCTGAGGAAGGACCTTTTACATTCCCTAATGGGGAGGCTGTGGAACATGGTGAGGAGAG
TAAGGATCGGACCATCATGCTGATGGGAGTGTCCTCACAGAAGATTTCTCTTCGGCTGAAGAGGGCTGTTGCCCACAAGGCCACCCAGACTGAA
TCACAGCCTTGGATTGGCAGTGAGGGCCCTTCAGGTCCCTTTGTGCTGTCTTCCCCAAGGAAGACAGCCCAGGATACTCTATATGTGCTTCCCA
GTCCCACCTCTCCATGTCCTAGCCCAGTCTTGGTCAGAAAGCGGGCTTTTGTCAAGTGGGAGAATAAAGACTCCCTCATAAAATCAAAGGAGGA
GCTCCGTCACCTCAGACTGCCTACCTACCAAGAACTGGAACAGGTACCTTCCTTTTCATTTTCCTTTTTTTCAGACTATCTTAGAGAAGTGGGG
CCAAACCAGGAAAGAGCATGTGGACTGAAGATTCAGATCAGGCTGATTACAAGCAGTCTAAAACAAAAAAGGAGGAGGGGAGAAACAGGGAGGG
AGAAACAGGGAGGAAGAGAGGGAGGGAAGGAAGAAAGGATGATTGATTCCAGCTTTCTCACAACCCACTGGGGGCCTTGGTTCACATTACACAA
GTAGCACAGGCTATACAGGGCTCCCTATCACCACACAGGTGGTGGCATTTCAGAGTGCTGGTGCCAGTGCCCAAAGCCCTGTTTAGTAGGCTCA
CAGAGAAAAACCACAGTATCTACTGGTTAGCTTTACTGTGCTGATACCACTCCTTTTCTAGATGGAACTAAAATGAACTTGCCTAAATCCTCCT
TCCCTAGAGGTCTCTTTAACATGTTCATCAGGGTTATGGTTTAGGATCATCATGGTTACCTAAGCATCTTCCTAAGAGAAGTTAGTGATCTTAG
TCCTATAGCAACAACAACAAGAGAAAGGTGAGTGTATTGTGGCTCACCATACAAAACGCCTCCCTCCTAAATATAAACGAAGAGAATCTTTTCT
AACACATCAGGGAATATGCTGATCTTTTACACATATTAGAATAGTGAAAGCAACAGTAGCAGGCAAACACACAGTGGAATATAATATAGAGCAA
TATAATCTATAAAATGTTAACAATCATGACAAGAGGAGCTGCTTTTCAATGTTAGGCTCACAATGTCCTAATTATCAGGTACCTAACAGAGACA
ATGGAAAAGACAAAATAAAGTTTACCAAGGAAAGGAGACCAAGAGTTTGGAAACATGGTGCCAAGGTTTGAGAGAAGGGAATAATGACATTAAA
AACAGGAAAATAGGGCTGGGCGTGGTGGCTCATGCCCCCAGCACTTTGGGAGGCAGAGGTGGGTGGATCACTTGAGGTCAGGAGTTCGAGAGCA
GCCTGGCCAACGTGGTGAAACCCCGTCTCTACTAAAAATACAAAAATTAGCTGGGCATGATGGCAGGTGCCTGTAATCCCAGCTACTCAGGAGG
CTGAGGCAGGAGAATCACTTGAACCCGGGAGGTGGAGGTTGCAGTGAGCTGAGACCAAGCCACGGCACTCTAGCCTAGGCAACAGACTCCATCT
TAAAAAAAAAAAAAAAAAAAAAAGGCCCGGCGTGGTGGCTCACGCCTGTTATCCTAGCACTTTGGGAGGCCAAGGTGGGCGGATCACGAGGTCA
CGAGATCAAGACCATCCTGGCTAACACGGTGAAACCCCATCTCTACTAAAAATATAAAAAATTAGCCAGGCGTGGTAATGGGCGCCTGTAGTCC
CAGCTACTTGGGAGGCTGAGGCAGGAGGATGGCATGAACCTGGGAGGCGGAGCTTGCAGTGAGCCGAAATGGCACCACTGCACTCCAGCCTGGG
CGACTGAGTGAGACTCCAACTCAAAAAAAAAAAAAGCGGGTGGGGAGGAGTAGGAGACGGGAACAGCAGAAGGAAAAAGAGCCAACAGCCAACT
GTCAAGATACTCAATTCTGAAGGTTCAATCATTACTATACTTCTTTTTGTTTCGAGTACAGTACACAACTCACAGGGCTGGCTCAAGAGGTTAC
CTATAATTGGTAAGAACCTACAAATGGTAAGAAACTACTGTCCCATGGCATAGTAGTTTCTTAATTATAGATAACTAAGCCATTTTAGAGACAG
GGAAGCAAGGATACGTTAATATCAACACTACTATTCACTTTATTACAGGAGGAGGAATGCCTTTCTGGCTTTTCCCAAAAACATCAAGGTTTGC
ACTGTGTTTTTGGAGACCTGTCAGTGCAAACACAAAACCAATATCCAGCTAGTAGAATTCCTTATATAATTCTACATTCTACAGACTGAAAAAC
TGTCCACGAGGGCTTCAGGATTCCCTGCATTGTCCTCAATGCTCTACCTTCCCTGTCAGACCCCCAGAAACTGAATTAGAGAAACATCTGAAGA
AACAAGTTAGATAACGTATGTGGAACTCTTGATTTGGTGCTTGGTACAAAGAACATAATATTAAGAGAAACATACGAAAAAAGAACAACAGATT
TTCTGGATGAATGGTACTCTCTTGCTCAGAGTTATTGTTAAGATGTGCTGTAAAAACCAAGTCAATGAGAAGGTGGCAAAACGTGGGTTAGACA
AACCCCTTCCTCTTTAGGGATAGAAACTGCCAAATGCAACTTCTAAAAATGATGAAAGAAACCTCGAAAAATGAACTCGGTAGAATAGAAAGAA
CTTTGGTCTTGGAGGCAAAGATTTGTGTTTTCAGTCCCAGACATTTCTTACACCAGCTGTTAAACTCTACAGCCTATTTGGTCCTTGTGAAATA
CAGGGCTGGGATTCCATGTTCTGAAAGGTTTGCCTCAAATCTTGACATTTTACATGCCGATCAAAAGGAAAAATGTGGTTGGCAACAATGCTTT
AAGAAGTGGCTCGCAGAAAGATTAGAATTCCTACTCAGCAACATCAGAGCCAGTAAACCCTCTCACCATTTTGACCACCGCCTCTCTTTACTTT
TTAGGCTTGTCAGATAGACCTAAGGTCCAGAATTCACTGTGTGTATTTTAGAGGAACAATTATGTGTCCCACTTTCCCATTAAGTATAATCCTT
ACAATGACTAGTCTATGTGTAATTCAACAGTCCTATCCATAATAACTAGTCAAATTAAATCAGTATTTACAATATATAACGTATATTAATAAGA
ATAAATATTATGTATTATTTTAAGTATAATGTACCACTGAACCAGCATGGGACAAAAGAATAAGCAAATAAAAGCTTTTATGTTTAGATATACC
AAGGCTCAACTCTGCAGTTTTATACTATATGGCAACATTTGCCTTTATAAGATTGAGGTATACAACCTATTTTCAGGGTGTTTGTGTAGGTTAA
GGCAGTCCATTATGTCACACACCGCAGGCACATAGCAAGTGCTCACTATTATATGGCAGCTGAGACAGCTATGTAGATAATTCAGATTGAGAAA
CAACCCCAGTGTATCCCAAATTACCCAGACACTGGCTATATATAATATTCTCTTGGAGTAATCAGTATAGGATAACATANNNNNNNNNNNNNNN
NNNNNNNNNNNNNNNNNNNNNNNNNNNNNNNNNNNNNNNNNNNNNNNNNNNNNNNTTGGCTCTATAATGATACCACTACAAATTAGACCACTAA
ATCTCAACTTAGGGGTGGGGTGAAATTCTTAACATCATCAAAAAGTTAAATTTGTTTTCAATAATTTGTGGGGGGGAAACTTAGATAAAATTAA
CTAACACCTTGCATTACAGAATTCTCAGTCATGCTTTCATGGCTCAGACCTGACCAGAGACTGTTTCTGCCAAGTTGAGATCTCCCGCGAGAAA
ACATCCAAAAGTTTTGTCATTATCTACCTATTGCTCGGAAATGGTTTATTCTGCCTGCTAACCACTCACAAACTTTGATATGATAACAAAGACT
AACTGCTGCTTACAAATGCTCGTTTGATATTTAACTTGCTATTTTCTATTCAGGGCAAAATAACTATCTTGAAAATGGTTGTTAATTCTCAGCC
ACAGTGAATAGGAAATGCCAATCAAAAGTAGCTCTCAATTTGTAATTCATTTGCTATTCATTGCTGCTTTATTAACATACCTCATTCTCCTTTC
CCTTTAGGAAATAAATACTCTGAAAGCAGATAATGATGCCCTAAAGATCCAACTGAAATATGCACAGAAGAAAATAGAATCCCTCCAGCTTGAA
AAAAGCAATCATGTCTTAGCTCAAATGGAGCAGGGTGACTGTTCTTAGCCCAGAAACTAAGTAGCACAATCTGTAGATGAGTATAGTGATCTCA
TTTCCTAAACTGTAATGCACAGACCTGAGGAACTTTACACTGACCAGCTTTAAAACAGTACTTTAAAAGGAAAAGCCTGTTACTGTTTATTTAC
CTAAAAGATTCCTAATGTGCAGCACTGTTTTCTCTTTCAGTTAGTTGACTCAAAGGGGGAAAACTAAAGAATGCAAAACTTTTGCTATTCATAC
CACTGATGTTCATCAAAAGTATGCGATCTAAAACATGACTATCATTTCCTGACAATGGGGCATCTCGGTGGCCTGCCTGGCTGATTCTTCCTTA
AAACTAAAATCTCTGGAAAATGGATTTGCTTCTTACCCTGTGTTTTCTGCAAACTCACTTACTTTGTTCCAGCCAAAGCTTGCTAATAATAGAA
AACTACCCATATTCCAAAAGTAGATTTCCTCTGTATCCCAGCATACTTTGTGAACCTGGCTCCTTCTTCACTACCTCAGATCTAATCAATTAGT
CCATGTACCCTCCTTCCTCACTACAGTCATAACACATGAGCATATCTACCTAGAAGCCAATTTCTACTGATGTAGCCACAACTTTTTAGAGGCC
TATTAAACATGACGTTATCCAATTGCAGGTCAACTTATAGTTGTAGCCTTACAACTTACAGGCATCAGAAAATAAGTAATCAAATTAGGTACCT
GGAAACATAGCTATTACCATCTCATATTACTGTCTAATTAAAATAAACATAACGCAAACATGTTTGTCCTTATATATTCTACAGTGGATAGAAT
TAGGAATTGATGGCTTAAAAAAAAAAAGTCTATGAAGAGTCTGTTTAACTCTTCATGTTCCATCTTTCTCTTCTGAAGTAAACTATTTTGAAAG
TTCTCTTTTTGAAATGAATTTGTGCTTAACTGTCTTCACTATTAATACTATTTAGAAATAAGCTAATTGGATCAGTGGCTTAAATAATAGCTGA
CTGTGTGTACATATGTATATAATATGTATATACAATATCAGGCATGCATGTGGCTTGGAATTTTGTTTCCTCCATAAAATGTGGAAGTGAATTA
AACAAGTTTTAGTCATTTATACAAAGTCACAAATATAAAGTTCAGTTTGTCACAAGATTAAATTGCTCACAAGGTAAAATTGTATTGTTTGGCA
AAATCACAAGTAACAATCCTGTGAGTTTTCTATTATGAAGGTTAATAATAAATGGGCTCATTTAGTTGCCTGGGCACCTATTCACAAATTCATT
TGTCAGCCTCTTTTTAGTTCTCTTAAAAAAAAAAAAATCATATGATCATTTTCCTTTTTGGGGGTACTTAGCTTCCATGCCTATAAAGTCTGGT
ACCAGACTGACTTGAAATTCATAAACAAGTTGTCCAATTGCCAAGAATATGTTAACAATTAAAAGTTCCAAACTAAAGCCAGTAGCACCAAGTC
TTCATAAGAATACAAAGTATACATACAGTATTGCTTACCTGGAGGATTCAGATCATTTAGGAATTCTCTTTGATGAAAGATCAGTTCCCATTTG
AGTTCCTCCTTGCACTGAGTTTTAGTGATATAGAACTAGCTTGTACTTAGTGTTTCATTACATTATAAAGAATAGTTTTACACACGTATTTACC
GTTTTCCAAATTTAAACTCAGAAATACCCAAAGCAGGCCTGCTTAAGCCCACTACCTGGCATATAAACTTATAGTAACACTTTGTTACTTTCTT
TTTAATAGGACAAGCATGAGTTAGGACAAACTCTAAAAATTCATATTCTTCACTATTCTTGTTTTCCTTTGATTGATATAGACCAAAGATGGTG
TACTCTAATTTTTTAAAACAGTAATGGAACACAATTTTTTTCATTCTTCCTCCTCTCCATTCGAAGTAAAGATCCCCAGTTAGTTTTTATATAA
ATAATCTATAGGGATTCAAAAGGTGTCACAGTCCACTTAATTAGTCAAATTAGCAATGGCTAAACAGTATCAAGTACTGCAGAATTTATCACTG
AAATGGATAAGAGGAAATAGTTTAGTCACAGGTTTTTACAGTCCAGCAAGGGCCAAAGAGGTATAGTATACAAGTTAATAGTATTTGTGTTGAG
CAACATGGGGCTAGTGGGATCACAGAAATCTGGAAAAAAAAAAAAAAGGCTTTGGCTTATCAAGCCTAGTGTAAATTTCTGCATCTCACACGAC
TTTAGTTTGGCCAGGTATTTATCTGCCAAAACAAGGACAAATCTTGTTGTATTAACAGCAGGGTCACTTCTCATTTTCTTTGCTGACTTACCTT
TTTACTGACCGTTGTGAATTTCTGTCTCAAAATGTATAATATAGAAATGCAAGAAAAAAACAAATGTACAGATTGTAAAGTTTTTTGATACCTA
ATGTAAGTTTTCTTTGTGTAATATTTATATGATAAAAGACATTAGGATCCCTACAACACAAATGTCTTGTGTTTCTTCTGCTTCGTGACCCCGT
AAACACTTTACCTGAATTTTTCCTATATTCCACAGCTAAGACCACTGCCCTATTCTATTAATCAAATTGAGTTTTCCTGGAAGGAAAGGCAAAG
CAGCTATTCAGTTTAAAATTTTCTACATCTTTTTACAACACGAAGAAGTAACACTACAGAAATATTCAATCTCTTTCCATCTTATTCATAACTT
ACAGTGGTCCCCAAATATTAAGTCTATAAATACTTAACATTAAAGGAGACAGACTTGAGGTTGTCCCTTCTCTGTGGCTTTTAATTCCACAGTT
TCATCTAAGCAAACTAGCATGTAAAGAAGAAATCCTTTGAAGCTATGGATAAAATTGGTAACGACAGCTCAATCCAGGTTACAGACCTACTCAG
GGCAATTTATTTGTGGTCATGATTATTTAGAATGATCAGACATAGCCACAATTTTCCCTTAATCCCCAGCTAATTTTTGTTTAACCACACCAAT
GAAGAAAGAGGAAAAATTTCAGTCACTCAAATTCCTCTGTGGAATGCATTCTGGAACGTTTAGATGGGGGTGTATTTATCATTTGGGCAATTCA
TAATACTTCATACAAAGTAATAAAGTCACACTGACAGCCTGATCCTGAAGGCAAGATTAAACTGCCTGCTCACCTCCCCCAATTTTCATCCCCA
CATTTCTCTAACCTGTGCTCCTTCCAAGGTATAGATAGTATCACTGCTCAGGACATTTTTCTGGTCACAAATTTTTTTTAGGTTGTCATGCCTC
CTCTTGTCACAGATACAGGTAATAAGATGTTTTTTATTTAAAATGTTTTCCTTTTTCTGTTTAATGAGGCAAGGTTTCCCTGATCTGCAAAACA
AAAGTAAGTTCAAAGAAAACCCTTGTTTCCCTTTTGATGTGTGCTTATGAGTAATAGTGTGGTGATTATGTTATAGACAGAATACATGCTCTTT
ATACGGTCGAGAGGAAAGATGACTACTTTAGGCAGAAACTCCCAGTAAGATCATGTCTATATTTGCTATTAGGTACCAGGCTGGACAAAAAGAG
AAGAAAAAAGGGGAAGGACAATGAGGTAATTAAAAAATGAAATTGAGAACCAGGCAAAAAAGAATGAAAAGGAGCTATCAAAAAAAGTGTTGGA
TTTTAGGAAGCAGTGGAGTGCCATTCATCATACTCCATGTCGTATTGCACTCAATTTATAAAACATTTTCACACTATGGCCTTTCCCATTAAGG
CCCCAAATTTCCTTCCTGCTATTCCAGATACTGCTCAGCAAACTGTTCTACAAAAGAAATTTTTACTGAACAAAGATTATCAGGAAAATCCCAC
CTTACAGGAAAGAGTAAAATATTAAGAGTCTACCTGATTCAGCTTTTCATTCAATTAAGGTTTATAGGAATGTGTACATAGGAGTGGTTTCAGC
TCCAACACGATTCCAGGAGCAAGTGCTAAACTTTGATAGGAAATAAGTGATTGTAGTGAATAACCCAAGCAGCACTCCTATAAAGGTACAAAGA
AATCTTATGGAAAATCTAATGAAACTGATAAACTATTGTTTCTGCCATATTTACCATAAACCTTTAGTGGTAAAAAAGTTGAAAGTAGTTGTAA
ATCTCCTTCATCAGCAACTAATGCTACCAAAAAAGAAGTGAGGCAGAAATGTAAAGGTTTTAAAGAAGATTTTTAAACAACTCTTGAAATATTT
TACTCAAGTAGTTTTTTTCTTAAAAGTTTTTAAAAATTATTTTTCAGAGTTCATAGCTACTGATAAAGGTATTTTCTTAATTATGGAAAGCCTT
TTTTTCAGCTATCACAAATGAAATTACATCTTTTCATTAAATATAAACACACAAAAAATAAATTTACAAATAACCAAAAAATTTTATATTTTCC
TACCTTGAACACATTTCTTCAAAGGGTTAGCTTGAAAAATACATGTCTAAGATTACAGAATATTCAATAGTTACTTTTAGGGTTTTAACATTAT
TTTAGATTTTACCACAGAGTTTACCAAAAAAATAACAACAAAAAAACCTGCTAAACATCAAATACCACTTGTCCACTGGGAACAATTCAGCTTT
CTATCAGTTGAGTTGGTTAGATCACATTAAGTTGTTATGTATAGGTATTGACCAGAAGCCTACAATGAGATACAGATTTTACACTAAGATTGAA
AAAATTTATGGTACCAGCTCTTTTTCTCTTAACTTTCTACACTAAGAGAAAAATACATTACTTGGCCTACATCTTAAACCATTCAAAATAGGCC
CTCTGTAAGTTTTTAACAAATTGTATTTGTATTTAGGTCTTTAAGAAACAACCTGAGATGATGGTAAGTGTCTCATTCCTCATTTGACAAATGA
GGACAAATCACTCCTTGAGAACTAAGGTCATTTCAATCCATAGCACCCCTGACCTAGTGCCATGATTCAAATTCACAACTCTTGATAGCTAAGA
CTTTGACTGCTCCAACTAGCAAATCACATATAGCCATTCCCTAACTTTGTGTTTCCCAAATCTAGCTGTGAATCAGAATTACCACAGGATGCCT
TAGCCCTACCCTTGATCTACTGAAGCAGAAAATTACTGCCTTAATTCTGTGTGATACAGGTCAGCCTGCCTTTTAATATTTGGATATCTAAAGT
GAGAGCATAAATGATGAAGAGACCAGTTTATAGGACTATTTCTCACACCGTCATGTAAAGTTGGTATGTGTCTTTGCTCCATTAAAGTATTTCT
GACCTCAATCTTAAACTTTGCTTTGTCAAACAAAATCATGCTGATTTCAAATCCAATTAGAAAATAAGTATGTACAGTAAAAAGTAAACTTGGG
CTTCAATTAATGAAACTTTAACAGAAACATGGCTAGAGAGGAATCTCCTCCTATAAGAGAAAAAAATGATTCACAACAACTGCCTACTAAATAC
AACTGGAAGTGCCTTTCCCTATTTTGTAAAATCGAATGAAAAACCCACAAAACACATCCACAGCAATACAGGTGAGGCAAGGGTCTATATTCCT
TTCTATTACAGGTGGGGTCTTTTATATGTCTCAAACCTGAACAAAATACAGGTAACAATCTAAATTCAATCATGACAATAAAATACTTTCCATT
TTCCAAGTCTGATGGTAATAAATATTTACATTTTAAAAAATAATTTTTGTTCTTTTTTTTGTTTTTTAAAAAGACACAACATGCTCAAGGGTAC
ACAGAATATGGAAAAAGTCATCACTTCATTTGAGAGATCAACTGGTAATAAAAAACAATGTTCTTACATATTAACCAATTCAAGAGTGGTATCA
CCAGGGAGTCTAACATGGTGTTTACTTCAAAGATGATATTCAAGTATGTTATTTGGAACACTATTTCTAATGTATCTATAAAGAAGCAAGCACT
GCTATCTAATTTTTATAGTTTTTAGTATCTTCTATATTCCACCTCCTCCACCACCACCACCACCACCACCACCACCTCCTCCATATGGATCTCC
ATAGCCTCTTCCACCATAGCCCCCTCTTCCACCATAACCCCCTCTTCCACCATAATCTCCCCTGCCTTGGAAACCTCCTCTACCANNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNCCCCAACCACCCCTTCCACCGCCGCCTTCTTGTCTCTTTTGCCAAGGGGGCATTTCTGGAGGTGGT
GGTTCAATTTCATTCGGCCGGCCTCCCCTGTCAGGCACCCTTCCCATCAGCACCTGTGTGAAGAAACATTTTCTTTTAGTTCCTAATATTTCAA
AAAAGTTTACATAAAAGTTTCAAGTGATGTTGTTTCTAATAAACAGAAGCCAATATTTAAGGACATGAAAGTACCAGACATGGCAGAACAAATT
AATATTAAAAATATTAAGTGTGGAAGCATAAGGAGAAATCTGTTAAAGGATACAAAATCACGGCTAGACAGAAGTAATAAATTCTAGTGTTCTA
TACCATTGTATGATGACTGTAGTTAATATATAGTTTCAAATAGCTACAAGGAGGATATTGAATGTTTCCAACACAAAGAGATGATAAATGTTTG
AGAGGGATATGCCAATCACCCCGTCTGATCACTATACATTGTATGTATCAAAACATCACTATGTACCTCATACATATGTACAATTATGGTGTTT
CAATTTAAAAAATAATTTTTTTACTTCCTGTTTACCAAGTCTGAAGAAAAAAGAAAAAAATGAATGGTTTTAAAAAGGAAATATGAAAAAATAT
AAAATGTGTGATACATGACAAATTCCTTAGTGTATATATATATATTTTTTTTTTTTTTTTTTTTTTTTTTGAGATGGAGTCTTGCTCTGTTGCC
CAGGCTGGAGTGCAGTGGCATGATCTCAGCTCACTGCAAGCTCTGCCTCCTGGGTTCACGCCATTCTCCTGCCTCAGCCCCCCGAGTAGTTGGG
ACTACAGGCACCCGCCACCACGCCCAGCTAATTTTTTGTATTTTTAGTGGAGACAGGGTTTCAACGTGTTAGCCAGGATGGTCTTGATCTCCTG
ACCTCGTGATCCGCCTGCCTCGGCCTCCCAGAGTGCTGGGATTACAGGCATGAGCCACCACACCCAGCCTAGAATATATTATTTTTAAACAATC
TGAGAGAGTATTTCTGAAACTTAAAGGCTTAATTTTTTTCTCACTTAAATTTGTTTAGCAAATTCTGTAACAAATGTAGTACCCATTTCACTAA
GAAGTTCTTTTAGGAAAACATTTTTTATATATATAACGACATACCTTACATTCATTAACTTAATATCCATTTATACAATAACAACTCTGGACTT
TCCATTGCCATTGATTTGGTAATTGATTATCCAATAAAAGATTATATTCCTAAGCACATTTCATTTCAAAAATGTATGAAGTACATATAACATG
GGACCTTAATGAGGTCAGAGATTGATATAAAAACTATAATTCCAGGTATCACTGCAAAGAGAGAGAAGGATTCTCAATGAACTGCTTCTTTTAA
GAAACTTAAGCTCACTTACTTTCAGTGATGATACTAGTACCATATTATTAAAAGGAAAACACAGTTAATATAATTCCTTTACTATGCTTTATAA
TCCTTTCAAAAATAAACTAGGGAAGAGTTTAAGTATCCAACCTTCTATAAAAAGTAGCAGCTTTATATGCCATGAGCAGAGGAAAGATAGCAAT
AAGAAGGGTGTCATCTGCTCACCACAGACAACGGGAAGCCAATAGCTTCCTGCTACTGCCTTTCAGGAAAGAAAGTAAAAAAGCAACTGCCTCA
ACCAAAATGACCAATTAATTATACACTTCCATTGATCCTCCAATCAAAACCAAGTTTAGGATTTATGTCACAAATGAAATATAAAAAGTGCAAT
ATCCTCAAAGTGAGACAAGGTTCTAAGTATGCAACATTCTTTTAAAGATGGCAATGATGTCCTAACTACCAAATCCAAAAAACAGCCTTTCTTG
GTTATTATTCTCACTGGTCTCTCTACCAAACATGGTATGTTGGTGGGCATATGGAGATGCCAGAACTGATCATAAGACTACCCTCAAATAATGA
GACCATAAATTATAAACTCAGAAGAGATGGCCAATGTTACTTACAGAAATAGAACTCTTGATCCAGCAAGACTAAATTATCACCCAATGCAAGA
TGTTAGGTAATATATTTAAAGAGAGAACACTTTCAGAACATAGGATTGAGCCACAGTGGAAGCACAGAGGCAAACACTGGTGATGCCTAACTTA
GCAATAGTTAGCAAAATGACTTTAAAAACAACCATCCCTCTGAAAAGGTATTTTATCTCAAGTTACACTGAGTGAGAAGCGATGCTTTAAAATT
ATCCAAATCCCTTATTTCTGATTTTTTGCTCTTTTGTAATAAAGTTTGCCATGGAGTGCTCTACCTAAATGAAGAGGACATGCCAGTTAAGCTG
ACATAAACATACTAAATTACTTTATTGTTATTGCTGTTGTAAAGTGAAAAATTCAGACTCCTTGAGCTTGTTTAAATAGACTACTGTCAGAGAA
ATGGATGTATACTCCAAATCAGGAAACAGAAGCTCAAAGAAGTTCAGTAGCTTGCTCAGGGTCATTTCTGTAAGTAAATCACAGAGCCAGGATG
GGAACCCAGATTGGTCTGACTCTAAAGCACAAACTCTTCTTCCCTTTACACCAAGGATATAACGAATCTGTCAGTACTTGAAACAATTCCGAAA
GATTCTTACTGACAAAAAATGATTACTTTTTAGAAAGATGGAACATAATTCTTGCTAAAGCTTTGACAAAAACAATTCATAATTCCTCTAATTC
AGAAAATACTTTGATTTATGAATACACAAATTCAAGTGAACATACACACATACACAAAAATAACATAACTAAGCCTAGACTGAACCCCCCTGCC
ACTCTTTTTTCTAAACATTTACCAGGACATGAAAGAAAAATAGACTAAAATCTGTAAAAACATTAAGTAAGATATCATCAATTCATTTTCCACT
GTACTGAAAGAAACACCAACCTTATTAATGGCACATGCGGTCTTCTCCCGGCTGGTGCCACTACTTGTCCTAATGATCTTGGATAGATCTTCAC
GAGCAGCAAGTAGATGTGCCATTGTAATCGTTGTCTTGGGTGACAAAGCATAACGCTTAGGCTGATAAATTCTTGCTATATCATCTGTTTTTAC
CTAAATGACAAGAGTTAAGGAAAAAAGGAACAAAAGTACATTTTAATGAAAGAGCAAAAACAATCATAATCTTTAGAAATAAAGAAAAGGAGAT
AATGCATAAGCAAGGAGTTAGAAAAGAGCAAAAACACCCAAATATCTGAGAATTTATGAATAATTTAGAACAAATAAAATAGACCAACAGGTGT
TAAAAATATGTAACTACCAGCAACAACTCGCTAAAAAATAATAACAAAAAAATTTCTATACATATTAACAAAAAATACCTAAAAAATAGTTTTA
CCAAAACCACATACAGGACATTTAAAAGGGAAACATCAGTAAATCTTGCAAAAACAAAAGGAAAACTTGTATAAATGGTAAGAAATACCAAGTT
TCTGTATGGGAACATTAAGTATTATTGTCAGTTTTCCCCCAAATTAACTTATAATCCTATATAAATAGTTTTTGAGAGAATGAAAAAAAAATCT
AAAGTTAAATCCAGAAACTCAATATGTAAGAATAACTAAAAGCATTTTGAAATATAATCCTACCAAATACTAATATGCTATAATACAAAATTAT
CATCATCTTATCAGTTAACATTTAATGAGAACTTATTAAATGTGCCAGGATGCTGTTCACTTAGTAAGGCATTATTTCTTTAATCCTCAAAATA
TCCTTTTAGAGTGGTTGGTTTTTATTATCCCCTTTTGCAGATGGAAAAAAAAAATGAGGCATAGAAAGCTTAAGTTAGTTTCCCCAAAATATAG
AGAGAATCCTAGCTCTGCCCACTTAGCAACAAAGGTCTACCTGACCCCCAAGCACAGGTCTAGTATAGATAACAGTTTGCTATCCTACCTCCAT
CTAATTAACTAGAACTTGGGAGAAAAAAACATAGATTGATGAAATAGTATATTGAGACTCAGAGCCTCATAAATCTATGAATGCAATAAATGAT
AAAGGAGACATCACAGATCAACGAGAAAAGGAAAGGATTATCTTTTAAATGGTGCTGGAATGACCTGGTGGCTATCTGTAATAATAACAATAAT
ACAGCACACCATATAACAAAGGGAATTCCAAAGAGAACAAAAAGTTAACTGTTTGAGGTTTAATTTTCAATTTAAAAACAACTACATTATAAAA
ATTAAAATTTCTGTACTTTAAAAGATATAAAACAACATCTAATGACAACTGGGGAAAATATTTTCAACAAATAATTCAACCAGCAAACTTCCTT
TCTATATAAAAACCTCAAACACAAAAAGAAAACAGAATTCTACAGGAAAAAAAAATGACAAGACATAAATAGAAGTTAAAAAGAGGAAATTCAA
ATGACTAATAAGCTTATGAAAACAGTTCAACTTTGTCAATAATAAGAACAGACAAAACAAGTTAGTCTTTTTAACATCAAGTTAGCAATAAAGA
GGCTAAAGTGACACAACATTTCATTTACCTTACTGGAAGGAGTTTAAATTCATATAAA
HUMAN SEQUENCE - mRNA
CCCGGGGGGGAGAGAGCCGGCAGGCGGCGGCGGTGGTGGCGGGGGCGATGCGCCGCGCCCGGCCGCGCTAGGTGAGCCGGCACCGGGAGCGCGG
GCCGCGGCCATGGGCACCCTGGGCAAGGCGAGAGAGGCTCCGCGGAAACCTTCCCATGGCTGCAGAGCTGCCTCTAAAGCAAGACTAGAGGCAA
AGCCAGCCAACAGCCCCTTCCCCTCCCATCCCAGCTTGGCCCACATCACCCAGTTCCGAATGATGGTGTCTCTGGGACATTTAGCCAAAGGAGC
CAGCCTGGACGATCTCATTGACAGCTGCATTCAATCTTTTGATGCAGATGGAAACCTGTGTCGAAGTAACCAACTGTTGCAAGTCATGCTGACC
ATGCACCGAATTGTCATCTCCTCTGCAGAACTGCTCCAAAAAGTTATCACCCTCTATAAGGATGCTTTGGCAAAGAATTCACCAGGACTTTGCC
TGAAGATCTGTTATTTTGTAAGGTATTGGATAACAGAATTCTGGGTCATGTTTAAAATGGACGCCAGCTTGACAGACACTATGGAGGAGTTTCA
GGAACTGGTGAAAGCTAAGGGTGAGGAGTTACATTGCCGCCTGATTGACACAACTCAAATCAATGCCCCTGACTGGTCCAGGAAACTTACTCAA
AGGATAAAATCAAATACCAGCAAGAAACGGAAAGTCTCCCTGCTCTTTGACCATCTGGAACCAGAAGAGCTATCCGAGCACCTCACCTACCTTG
AGTTCAAGTCTTTCCGGAGGATATCGTTCTCTGATTATCAGAATTACCTTGTAAATAGCTCTGTGAAGGAAAACCCCACCATGGAGCGATCTAT
TGCTCTGTGCAACGGCATCTCCCAGTGGGTACAACTGATGGTTCTCAGCCGCCCCACGCCGCAGCTCCGAGCAGAAGTCTTCATCAAGTTCATC
CAGGTGGCTCAGAAGCTCCACCAACTACAGAACTTCAATACACTGATGGCTGTGATAGGTGGGCTGTGTCACAGCTCAATCTCGAGGCTCAAGG
AGACAAGTTCGCATGTCCCACATGAAATCAATAAGGTTCTCGGTGAGATGACTGAGCTGCTGTCCTCCTCCAGAAACTACGACAATTACCGGCG
AGCCTATGGAGAGTGCACCGACTTCAAGATCCCCATTCTGGGTGTGCATCTCAAGGACCTCATCTCCCTGTATGAAGCCATGCCTGACTATCTG
GGGGACGGGAAAGTGAACGTCCATAAGCTACTGGCCCTATACAATCATATCACTGAATTGGTCCAGCTGCAAGAGGTGGCCCCACCCTTGGAGG
CTAACAAGGACTTGGTACACTTGCTGACGTTATCCCTGGATCTTTACTACACTGAGGATGAAATCTATGAGCTTTCCTATGCCCGGGAACCAAG
GAACCACAGAGCTCCACCACTAACACCTTCAAAGCCACCAGTAGTAGTGGACTGGGCTTCTGGAGTGTCTCCCAAACCTGATCCAAAAACCATT
AGCAAACACGTCCAGAGGATGGTGGATTCTGTCTTCAAGAACTATGATCACGACCAGGATGGATACATTTCTCAGGAAGAATTTGAAAAGATTG
CTGCGAGTTTTCCATTTTCCTTCTGTGTGATGGACAAAGACAGGGAAGGCCTCATCAGCAGGGATGAGATCACAGCCTACTTCATGAGAGCCAG
CTCAATCTATTCCAAGCTGGGCCTGGGCTTTCCTCACAACTTCCAAGAGACCACCTACCTGAAGCCCACTTTTTGTGACAACTGTGCTGGATTT
CTCTGGGGAGTGATCAAACAAGGATATCGATGTAAAGACTGCGGGATGAACTGTCACAAACAATGCAAAGATCTGGTTGTGTTTGAGTGTAAGA
AGCGAGCCAAGAACCCAGTAGCTCCCACAGAGAACAACACTTCTGTGGGGCCAGTGTCCAACCTTTGCTCATTGGGAGCCAAAGATCTGCTCCA
TGCACCTGAGGAAGGACCTTTTACATTCCCTAATGGGGAGGCTGTGGAACATGGTGAGGAGAGTAAGGATCGGACCATCATGCTGATGGGAGTG
TCCTCACAGAAGATTTCTCTTCGGCTGAAGAGGGCTGTTGCCCACAAGGCCACCCAGACTGAATCACAGCCTTGGATTGGCAGTGAGGGCCCTT
CAGGTCCCTTTGTGCTGTCTTCCCCAAGGAAGACAGCCCAGGATACTCTATATGTGCTTCCCAGTCCCACCTCTCCATGTCCTAGCCCAGTCTT
GGTCAGAAAGCGGGCTTTTGTCAAGTGGGAGAATAAAGACTCCCTCATAAAATCAAAGGAGGAGCTCCGTCACCTCAGACTGCCTACCTACCAA
GAACTGGAACAGGAAATAAATACTCTGAAAGCAGATAATGATGCCCTAAAGATCCAACTGAAATATGCACAGAAGAAAATAGAATCCCTCCAGC
TTGAAAAAAGCAATCATGTCTTAGCTCAAATGGAGCAGGGTGACTGTTCTTAGCCCAGAAACTAAGTAGCACAATCTGTAGATGAGTATAGTGA
TCTCATTTCCTAAACTGTAATCCACAGACCTGAGGAACTTTACACTGACCAGCTTTAAAACAGTACTTTAAAAGGAAAAGCCTGTTACTGTTTA
TTTACCTAAAAGATTCCTAATGTGCAGCACTGTTTTCTCTTTCAGTTAGTTGACTCAAAGGGGGAAAACTAAAGAATGCAAAACTTTTGCTATT
CATACCACTGATGTTCATCAAAAGTATGCGATCTAAAACATGACTATCATTTCCTGACAATGGGGCATCTCGGTGGCCTGCCTGGCTGATTCTT
CCTTAAAACTAAAATCTCTGGAAAATGGATTTGCTTCTTACCCTGTGTTTTCTGCAAACTGACTTACTTTGTTCCAGCCAAAGCTTGCTAATAA
TAGAAAACTACCCATATTCCAAAAGTAGATTTCCTCTGTATCCCAGCATACTTTGTGAACCTGGCTCCTTCTTCACTACCTCAGATCTAATCAA
TTAGTCCATCTACCCTCCTTCCTCACTACAGTCATAACACATGAGCATATCTACCTAGAAGCCAATTTCTACTGATGTAGCCACAACTTTTTAG
AGGCCTATTAAACATGACGTTATCCAATTGCAGGTCAACTTATAGTTGTAGCCTTACAACTTACAGGCATCAGAAAATAAGTAATCAAATTAGG
TACCTGGAAACATAGCTATTACCATCTCATATTACTGTCTAATTAAAATAAACATAACGCAAACATCTTTGTCCTTATATATTCTACAGTGGAT
AGAATTAGGAATTGATGGCTTAAAAAAAAAAGTCTATGAAGAGTCTGTTTAACTCTTCATGTTCCATCTTTCTCTTCTGAAGTAAACTATTTTG
AAAGTTCTCTTTTTGAAATGAATTTGTGCTTAACTGTCTTCACTATTAATACTATTTAGAAATAAGCTAATTGGATCAGTGGCTTAAATAATAG
CTGACTGTGTGTACATATGTATATAATATGTATATACAATATCAGGCATGCATGTGGCTTGGAATTTTGTTTCCTCCATAAAATGTGGAAGTGA
ATTAAACAAGTTTTAGTCATTTATACAAAGTCACAAATATAAAGTTCAGTTTGTCACAAGATTAAATTGCTCACAAGGTAAAATTGTATTGTTT
GGCAAAATCACAAGTAACAATCCTGTGAGTTTTCTATTATGAAGGTTAATAATAAATGGGCTCATTTAGTTGCCTGGGCACCTATTCACAAATT
CATTTGTCAGCCTCTTTTTAGTTCTCTTAAAAAAAAAAAAATCATATGATCATTTTCCTTTTTGGGGGTACTTAGCTTCCATGCCTATAAAGTC
TGGTACCAGACTGACTTGAAATTCATAAACAAGTTGTCCAATTGCCAAGAATATGTTAACAATTAAAAGTTCCAAACTAAAGCCAATAGCACCA
AGTCTTCATAAGAATACAAAGTATACATACAGTATTGCTTACCTGGAGGATTCAGATCATTTAGGAATTCTCTTTGATGAAAGATCAGTTCCCA
TTTGAGTTCCTCCTTGCACTGAGTTTTAGTGATATAGAACTAGCTTGTAGTTAGTGTTTCATTACATTATAAAGAATAGTTTTACACACGTATT
TACCGTTTTCCAAATTTAAACTCAGAAATACCCAAAGCAGGCCTGCTTAAGCCCACTACCTGGCATATAAACTTATAGTAACACTTTGTTACTT
TCTTTTTAATAGGACAAGCATGAGTTAGGACAAACTCTAAAAATTCATATTCTTCACTATTCTTGTTTTCCTTTGATTGATATAGACCAAAGAT
GGTGTACTCTAATTTTTTAAAACAGTAATGGAACACAATTTTTTTCATTCTTCCTCCTCTCCATTCGAAGTAAAGATCCCCAGTTAGTTTTTAT
ATAAATAATCTATAGGGATTCAAAAGGTGTCACAGTCCACTTAATTAGTCAAATTAGCAATGGCTAAACAGTATCAAGTACTGCAGAATTTATC
ACTGAAATGGATAAGAGGAAATAGTTTAGTCACAGGTTTTTACAGTCCAGCAAGGGCCAAAGAGGTATAGTATACAAGTTAATAGTATTTGTGT
TGAGCAACATGGGGCTAGTGGGATCACAGAAATCTGGAAAAAAAAAAAAAAAGGCTTTGGCTTATCAAGCCTAGTGTAAATTTCTGCATCTCAC
ACGACTTTAGTTTGGCCAGGTATTTATCTGCCAAAACAAGGACAAATCTTGTTGTATTAACAGCAGGGTCACTTCTCATTTTCTTTGCTGACTT
ACCTTTTTACTGACCGTTGTGAATTTCTGTCTCAAAATGTATAATATAGAAATGCAAGAAAAAAACAAATGTACAGATTGTAAAGTTTTTTGAT
ACCTAATGTAAGTTTTCTTTGTGTAATATTTATATGATAAAAGACATTAGGATCCCTACAAAAAAAAAAAAAAAAAAAAAAAAAAAAAAAAAAA
AAAAAAAAAAAAAAAAAAAAAAAAAAAAAAAAAAAAAAA
HUMAN SEQUENCE - CODING
ATGGGCACCCTGGGCAAGGCGAGAGAGGCTCCGCGGAAACCTTCCCATGGCTGCAGAGCTGCCTCTAAAGCAAGACTAGAGGCAAAGCCAGCCA
ACAGCCCCTTCCCCTCCCATCCCAGCTTGGCCCACATCACCCAGTTCCGAATGATGGTGTCTCTGGGACATTTAGCCAAAGGAGCCAGCCTGGA
CGATCTCATTGACAGCTGCATTCAATCTTTTGATGCAGATGGAAACCTGTGTCGAAGTAACCAACTGTTGCAAGTCATGCTGACCATGCACCGA
ATTGTCATCTCCTCTGCAGAACTGCTCCAAAAAGTTATCACCCTCTATAAGGATGCTTTGGCAAAGAATTCACCAGGACTTTGCCTGAAGATCT
GTTATTTTGTAAGGTATTGGATAACAGAATTCTGGGTCATGTTTAAAATGGACGCCAGCTTGACAGACACTATGGAGGAGTTTCAGGAACTGGT
GAAAGCTAAGGGTGAGGAGTTACATTGCCGCCTGATTGACACAACTCAAATCAATGCCCGTGACTGGTCCAGGAAACTTACTCAAAGGATAAAA
TCAAATACCAGCAAGAAACGGAAAGTCTCCCTGCTCTTTGACCATCTGGAACCAGAAGAGCTATCCGAGCACCTCACCTACCTTGAGTTCAAGT
CTTTCCGGAGGATATCGTTCTCTGATTATCAGAATTACCTTGTAAATAGCTGTGTGAAGGAAAACCCCACCATGGAGCGATCTATTGCTCTGTG
CAACGGCATCTCCCAGTGGGTACAACTGATGGTTCTCAGCCGCCCCACGCCGCAGCTCCGAGCAGAAGTCTTCATCAAGTTCATCCAGGTGGCT
CAGAAGCTCCACCAACTACAGAACTTCAATACACTGATGGCTGTGATAGGTGGGCTGTGTCACAGCTCAATCTCGAGGCTCAAGGAGACAAGTT
CGCATGTCCCACATGAAATCAATAAGGTTCTCGGTGAGATGACTGAGCTGCTGTCCTCCTCCAGAAACTACGACAATTACCGGCGAGCCTATGG
AGAGTGCACCGACTTCAAGATCCCCATTCTGGGTGTGCATCTCAAGGACCTCATCTCCCTGTATGAAGCCATGCCTGACTATCTGGGGGACGGG
AAAGTGAACGTCCATAAGCTACTGGCCCTATACAATCATATCAGTGAATTGGTCCAGCTGCAAGAGGTGGCCCCACCCTTGGAGGCTAACAAGG
ACTTGGTACACTTGCTGACGTTATCCCTGGATCTTTACTACACTGAGGATGAAATCTATGAGCTTTCCTATGCCCGGGAACCAAGGAACCACAG
AGCTCCACCACTAACACCTTCAAAGCCACCAGTAGTAGTGGACTGGGCTTCTGGAGTGTCTCCCAAACCTGATCCAAAAACCATTAGCAAACAC
GTCCAGAGGATGGTGGATTCTGTCTTCAAGAACTATGATCACGACCAGGATGGATACATTTCTCAGGAAGAATTTGAAAAGATTGCTGCGAGTT
TTCCATTTTCCTTCTGTGTGATGGACAAAGACAGGGAAGGCCTCATCAGCAGGGATGAGATCACAGCCTACTTCATGAGAGCCAGCTCAATCTA
TTCCAAGCTGGGCCTGGGCTTTCCTCACAACTTCCAAGAGACCACCTACCTGAAGCCCACTTTTTGTGACAACTGTGCTGGATTTCTCTGGGGA
GTGATCAAACAAGGATATCGATGTAAAGACTGCGGGATGAACTGTCACAAACAATGCAAAGATCTGGTTGTGTTTGAGTGTAAGAAGCGAGCCA
AGAACCCAGTAGCTCCCACAGAGAACAACACTTCTGTGGGGCCAGTGTCCAACCTTTGCTCATTGGGAGCCAAAGATCTGCTCCATGCACCTGA
GGAAGGACCTTTTACATTCCCTAATGGGGAGGCTGTGGAACATGGTGAGGAGAGTAAGGATCGGACCATCATGCTGATGGGAGTGTCCTCACAG
AAGATTTCTCTTCGGCTGAAGAGGGCTGTTGCCCACAAGGCCACCCAGACTGAATCACAGCCTTGGATTGGCAGTGAGGGCCCTTCAGGTCCCT
TTGTGCTGTCTTCCCCAAGGAAGACAGCCCAGGATACTCTATATGTGCTTCCCAGTCCCACCTCTCCATGTCCTAGCCCAGTCTTGGTCAGAAA
GCGGGCTTTTGTCAAGTGGGAGAATAAAGACTCCCTCATAAAATCAAAGGAGGAGCTCCGTCACCTCAGACTGCCTACCTACCAAGAACTGGAA
CAGGAAATAAATACTCTGAAAGCAGATAATGATGCCCTAAAGATCCAACTGAAATATGCACAGAAGAAAATAGAATCCCTCCAGCTTGAAAAAA
GCAATCATGTCTTAGCTCAAATGGAGCAGGGTGACTGTTCTTAG

TABLE 5
NOMENCLATURE
TCSGNM Nmyc1
Celera mCG19753
HUMAN NOMENCLATURE
HGNC MYCN
Celera hCG1783900
MOUSE SEQUENCE - GENOMIC
TAATGAAATTCTGAAGCCTACAGACTCTTCCTAGGGGAGAGAGGAAGTATTCAACTTGGTAGAAAATAAATGATGGGTGAGGGAACAGGAGGAT
GAAAGGGCCTGATGAACAGGTGAGCGCCTATGACTCCGTGGGGTGGAGGTGATGTTCTGGATTATCTAACCCAATCTCCCTCATAATGTTGTTC
CGGGACAATCGAATTCATCCAGAAGGATCTTGCCCAAGGTAGGTAAAGGGGATTCAGAGAAAACTTTTCTATGCTTTGGTGCTATGAGATAGGA
GAGCAAAGAAAAATCACGAAGACTTTGGTCAGCAAGCCAAATGCCACATTTGAGGGTGTCTTGTCCTCAACCCAATTCTAAAGGGACCCTTGTG
TTACTGTGCCCAAGAAGTTGGAATTTCCCCCAGGAGTTTCACTTTAGGGGAAGAAAAAGCCTTGAGTCCTGGTTCCAGCCAGAGAGGGAGCTTG
CCTTCAGGGAAGTGATTGTCCAGATCTCACAGAGTTCTTGCAAATTGATATTACAGCACCTTGCAAGAGAAAATGGAGCTGAGACAATGCTGCC
TCAAAGGTGGGTGGGAAAGACCAGGACTCTTGTGTCACAAATACCTGTTTCCTTTCATTTAGTCCTATACCTGTCTGGACATCGAAGATGAACT
GAGGGCCTGGGATGGGGAAGGGGGTGGGGGGTCATTGAACCCCTTTATATTTGTTTCTCTCTCTGATGTGATCACATTGAATGCACCTCTGTTT
TCCACCATTGATTGCTAGGCTGCATCATTGGTCTATTGAGGACGAGTAGTCAACTCTTGCTTGTTAACGGCTGCCTTACAACTATGATTAGAAC
GAGCATTTAAAATGGTGTTACGGGGCTGGAGAGATGGCTCAGAGTTAAGAGTACTGACTGCTCTTCCAGGGGTCCTGAGTTGCTCTCCCAGAAA
CCACATGGTGGCTCACAACCATCTGTAATGGGATCTGTCTGGTGTGTCTGAAGACAGCTACAGTGTACTCATATAAATAAAAAAAAAAAGAAAT
TAAAAAGAAAAAACTTTAAAAATACACATTAAAAAAAAAAAAGATAAAATGGTGTTACCACCCTTTCCCAGTGCCCTTTTCCGATGATGGCGCT
GCATGGTGAAAAGCTTGAGTTAGTACTTGTAGCTATGCTGTCCAAGGATCCGCTGCAATCCGTGCAGGTCTTCGGACACAAGAAGACAGTCACT
GCTGTGGCCCACTGCAAATGAGGAAACGGGCTCATCAACGTGAATGGATGAAGCCTCTAAGAAGGAGATCAAAGATAGCCTCATTCAGTATGAT
CGGACCATTCTTGTAGCTGACCTCCAGTCGCTGCGAATCCAAAAAGGTTGGAGGTCCTGGTGCCCGTGCTTGATACCAAAAATCCTACCAATAA
GCCCATCCCAAGGACCAGGGTTTGTTTACCTCTGTAATGCACATCCTAGGATTTTTTTTTTAAATGACATTACCGATACAACCCCTCAGCCACA
CACTCACCCTTGGCTAAACTCTCTCGGTGCTAGCTTTGAAATTTCCTCCAGCCTCCAATGGCAAAATACATGCACCTAGTTTGGTGGGGCACCT
CAAATATTATAAACTTCATTCGGCTGTGGTGGTCAGCTTGTGATTCAAATTCAACTACCACCTAATTTGTCTCATCTCAGCCTCTCTGAACCCT
TCAGAGAGTTCAGTGGCAGGTAGTCAGGAAGAGTAACAGCAAATCTGGGTAGGAAAACATAGACTTAAGGGTAGTTTAGGCCGGGCGTGGTGGC
GCACACCTTTAATCCCAGCACTTGGGAGGCAGAGGCAGGTGGATTTCTGAGTTCGAGGCCAGCCTGGTCTACAGAGCGAGTTCCAGGACAGCCA
GGGCTATACAGAGAAATCCTGTCCCGAAAAACAGAGAGAGAGAGAGAGAGAGAGAGAGAGAGAGAGAGAGAGAGAGAGAGAAGAGAAGAGAAGA
GAAGAGAGTCACCACCACCACCCAATCCCGAATGTTATTCTAATCAAAACAAACAAAACAGAACAAGCAACTTTGGGGGAGAATGGGTTTATTT
TAGCTCACAAGCGCACGTTACAGTTCACTCTACGGCAGGGAAGTCAAGGCAGCAAGAAGTTCAAAGGGCTGGTCCATCCACAGTCAGGAGCAGA
GGGCGAATGAAGTATACTCGACATTCCGTTCACTGTTTCCACTCGAATACAGCTCAGAACCCAATCCCAGAGAACGGTACCACCCACAGTGGTC
GAGGTCTTCCCACATCAATTGAGGCAGTCACTCACAGACATACCCACAGGCCAACGTGACCTAGACAATCTCTCATCAAGACTCTCTGCCCAGA
TGATTCTAGGTTGTGTCTAGTTGATAATTAATCTCACCATCATAGGTATTAGAGATAGATGTAAATANNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNTATATATATATATATATATATATATATATATATATATATATATATATATATATACATACACATA
TATATATACACACACTTAAAAACAAATAAGCATACCTCAAAATCTAGCAAGCAGCATAGCCTGGCAGCACAGTGCAGTGTAGAGTATCTGGTGT
TTATCCTCAAGACTGTAAGGCTTACTAGAGAGTATAGCTTGCTATCTCTGCCCAGGGCTGTTGAAAGAGTACCATATGTTGTTGGTTCTGAGAA
AGTATTATTATTGGAGGGGGTAGGGGTGTCACTCATGCTACAGCATGCCTGTGAAAGTCAAAAGACAATTTTGTGGCAGTCAGTTCTCCTCTTC
CACCGTGCAGGTCCCAGAGAACCAACTCAGGGTGCTGTGCTTGGTAGCAAGTACCTTTATCCAATAAGCCATCCTGCTGGCGTGTGTGTGTGTG
TGTGTGTGTGTGTGTGTGTGTGTGTGTGTGTGTGTGTGTGTGTGTTTTGGTGGCATTGGTTGGGGGGGGGGGGTGTATCTTTGTTTTGAGATAG
GATTTTGCTACACCACCAAGGCAAGCCTTCAACTTCTATATGTAACCTAGGTTGGCCTGATTAGTTAGCTCAGGCTAATACCAAATTTGCATTT
CTTTAGACTCAGCCACAATAGCACTAGGATCACAGGCAACACACCCTGCTTGTTGATTGGCACTAACAAGTCTTACAGTGTTTTTGCTATTGAC
AACCTTGAAGCAGTACGTATTCTTGGTGATTTGTCTTAATCTCTCTCTCTCTCTCTCTTTCTCTCTCTCTCTCTCCCTCCATCCATCCTTCCCT
CTCCCTCTCTCTGTCCCTCTCTCTGTCCCTCTCTCTGTCCCTCTCTCTGTCCCTCTCTCCCTCCCTCCTTTCTTCCCTCCCTTCACCCAGGGTA
TGACTTAGTCTGTAAACCACTCTAGTCTCAATTTATGTAAATCTACCTTCTTTGGCCCCCGATGTTGAGTGTTGGGATTAAAGATGAATGTCAC
TAATGGAGTCTCAGTTTGGTTGTTGTTGTGGTTATGGTTTTGGTGTGGTTTTGTTTTTGTTTGTTTGTTTGTTTGTTTGAGACAGGATCTTGCT
AAAGACACCACATTAGCCTCAATTTTGTAAATCTTCCCAATTCTCCCTCCCAAGTGTTGATGTTTACAGATATGCTCTGTTATCTTCAGGAGTT
TTAAAAACTATTTTACATTGCACACTTATTTATTTTAAGGACTGGGTGAGATCATAGGACAGCTTTAAGCAGATGATTCTCTCCTTCCACTAAA
TGAGACCCAGGGATCAAACTCAAGTTGCCAGGTTTGGCCACAGGTTCCTTTACCCACTAAAGACATTTCAGCAGCTCTTGCTTTATTATCTCTT
TTTGGGTGATTCTAGAATGGAATTCTTTCTACTTTTGGGACAGAAGCAGCAGTGGTGTATGAAAGTGTTCGTATCTCTCACTCTCCAGCCTCAC
CACCGTTTTGAGGCAATCTGCTAGACTGAATTGTTAATCTCCCTGAGATTTAATTTGGATTGTCCCGAGTCAGGCGAAGTAGCCCATTTTCTTG
TTGGTACTCAATTTGCATTTCCTGTTTGGCAAATTGCCTATTCATATCCTAGAGCCAGATTCCTCTCTGGCTTTTCTTTACCTCCATTCAGTTT
CTAGGATCCCTTCCTGCACGAGGAATATAACCTTTGCCTCTGAGGGCCCTCAGGAAGTGCCATCCCAAATATGACATTTTTGAGAGCTGATGAA
CTCAAGGGGCGACGGAGCTGGAAACTGCAAGTGTAGGCTTAGCTGTACCCCTTATCTACATAAAGACATCCTCCCAAGGCAGCTAGGCTGTTCT
GAATCTACTCTCCAGTTTCTTATCTCCCAGTGGAGATTACCTCCGTGACACCCAGCCAAGGTAGACTCTCCCTGACCTATTCTGTGGGCCGCTT
CCAGACAGGGTTAATAATACCTGAGACTGTATCGGTCTGATAAAGTCAGACCTGCTTTCCTTCCCTCAGCTCCCAGAACGCTAGGCATCCCGGG
ACACTGCATGAGTCTCAGTCATTTGACCGTCCTTCAGACCTCAGATGTGTGAGGCTCCCATCCACTGACATATTGTTCAGTGTATTTTCTACTA
TGCTAATCTGTCTGTTTTGATAGTTCTACTTTTTGCAGTCAAATCTTAAAAGCCAACCTATTTTCATATTTTACTTTTTTTGTGCAATGCAGAA
TATAAGTTTTCTTTTCTCCTCTTCCTCCTCTGTCTTTTTTTCTTTCATACAATACTTCCTCATCGTCCTCCCCTATCCCCGACTTTTTAAAAAA
ATAAAGTCAATTAATCTGCAGGATTTTTTTTTTTTAGTATGTATATGTGTTGACCTGTTTGCCTCAGAATGCATGCAGAGATCAGCAAACAACT
TGCAGGAGTCTTTTCTCCCCTTCCACCACGTGCATGGGGCCTCAGGCAGTTAGACATAGAGGAAAGGGTCCTTACCACCTGAGCTAGCTTACCA
ACTGGGTCCCAGGGATTTTTTCAACTATTTTAAGCAAAAATTACAAGTCCTTTATTTTGTTGGGCCTGGGTCTTGTGGTATAGTCCAGGATGAC
TTCGGACTCCCTGCCTCAGCCTCACGAGGGCTGGAGTACAGTACCACAAGGGACTGTGCCTTTATTTGGGCAGGAGAAATTTGGACTCAGTAAA
ATAGTCCCAGAAGAAACCCAAAAGCTGGATGAGGTTGTTGGTCTACTAACCATGATGGATTAAATCCTCCTGGGTTTGTGTACCTGTGAGCTGT
CTTTATTTATTTCATTTGGTAAAGAAAGANNNNNNNNNNNNNNNNNNNNNNNNNNNNNNNNNNNNNNNNNNNNNNNNNNNNNNNNNNNNNNTAA
CCAGGTCACCTTCTCTCTGTCCCCTCTTTTCTGAAACATCTTATCTTTTAGCAGAGTGAAGAGCACAGGTGTGGAGAGAGAACTTTATCCATAA
CTAACCAGGCCATCTTAGCCGACTTTCCCAGTCCCCTGCTGCTTGAGGGAGTTCTAGTAGGCTGGTCAAAGTGTCCTTTACGTGTCCGCTGCTG
AAGTTCTGAGGAAGATTCTAGAAGCCAGATGTGGTGGCACAGACCTGTAGTCCCAGAGTTTGGGTAATGGCAAGAGGATCAATTCAAGGTCATC
CTCCTCAATAAAAATAGAGTTCAAGGTCATCTTGGACTATATGAGACCTCGGCTCAAAAAACAAACAGAAACACCTCTCCTTCCCCTCCCCCCC
CCCCCAAAGCAACAACAAAAAGAGAAGGTTCTGGGAGCCACACCTATCTCTCCCCCACTGTGCCACTAATAAACAAAACTAACCTGCCAGATCT
GGCTGTCTAAAAGGGTTCCTTTCAATGGAGCCTACCTCCAGCAGCATCCAGGAACACTTACCATACAGCCCCTGCTGTGTATGTATAATTACAA
TCCGAGGCAAAGTTTTCAGAGAGCCAGCATCTTGAGGGAGAATTCAGCGGGAGGTCTCGGTTTGGAAGAGGCCAGGGGTGTGAAAATGAGGGTG
TTAAAGGCTTTTGCAAAGGTAATGGGTTTTACATATGTGAGCCCAATAAGAAAGGCCTTGGAAGGGTGGCTCACCCTCATTCACGGCTACTTCC
TAGAACCTGCCGCAGTCCCCAGAATTATGTAGGCATTCGGGGACAGATGGGAAGACTTTCACACATGCTAATACACATTTTGACCTTGACTCTC
TAGGCATTCTCTTCTTCCTCGCTGGAGGTGAATGAGGGGTGTCATTTTCAATTTAGAGGGAAATGGAGACGCTTCCCCACAAACAAGCTTAAGG
GTTTAAGGATAAATCCCACCCTACTGAGAATGTGTGTTGAAAAAGGGCTTCACCCAGGGACCCAGGTTACTCCTGGAAAGTTTTGTGGGGCAAG
AGCATTTATTTTTAGCCCCACATTTAGGAAACAGACCTTTTAGCATGCTTGCCTAGACAGAAGTGAGACGCTGTGGGATGGGGAGGGAGGGAAA
TGTGCCCACGGCTGTGAGGAAAGAATACACGCACCCTGCTCTAGTCTAAATAAGATGTCGCGCTCTCTCTGTTCACGCCACCTCGTAAGTGAGG
CAAATGGGACACAAAGAGGCCAAATTAATGATGGCTTTCCTTGATGTCACCATCCGTAGGAACTCGAGGTATACAGAGCAGTCAGTGATTTGGG
CAAGGAAAGCAGCGGTCCGTAATTCAACCCTGGCTTCGGAAAGGCTTTTGTTGGGAACGGTGGGCGCGACTTTAGGGGACGTCCCTCCACGCGT
TTCACAATCCCTTCCGCGGTAGCAAGGATTCGCTAATTGTCTCGCGGGGGTGGCCCCACTCGGGCTCCGAGGCTGGGCGGTGCGGCTCCGCTTT
CTGCTTGGTCTCCGCGAGGTGTCGCCTTCGGCCGAAGAAAGCGGTAAGGCGCCACCCTCGTAGCCCGCACTTATTTATTTTCAAACAAGGGGGG
CGCCCCTTCCTTTCAATTTGAAACTAGAAACCTCCTGCGGTCTCGTTTCTACGTGGGCGCGTCCTCGGCCTCCCATTTTGCACTCCTGTGTCGC
CTTCCTTCGAGATCACACACGAACAGCCCCTCGACCAGGCCTTGTTCCACTTTAGCCTGGAGCTCATCTTTCACACAAACACACACTCGCGCGC
AGTCCACGCAGGGACTGGGCTAGAAACCTCTTTTCCCATTCCCAGAACACACCCAGCGACCTTTAAAAAAAAAAAAACACGGAGTGCAGCGGGT
GCAAGCCAGGGGGTCCAAAAGGGAGGGGGAAGCACTCCTCGGGTTTCCCAGCTTTGCTGCTTGCAGTCTGAAAAACGAGAGGGGGGAGGGGGAG
AGAGAGGAGGGGAGGAGGGGGAGAGGAGAAGAGGGGAGGAGGGAAGGGAGAGAGAGAGAGAGAGAGAGAGAGAGAGAGAGAGAGAGAGTCGGAA
AAAAAAGAGAAAAGCAAATGGCTTTTGGCGCGAAAGGCTTTGGCGCCTCCCCTGATTTTTATGGAAATCAAGAGGGCGGGGAAAAGCCTCTTGC
CTCTGCCTTTCTCCCTCCCCCTCGTCTGCGCCACAGCCCCCTTCTCTCCCCGCCCCCCGGGTGTGTCAGATTTTTTCAGTTAATAATATCCCCC
GAGCTTCAAAGCGCAGCCAGTGACAGNNNNNNNNNNNNNNNNNNNNNNNNNNNNNNNNNNNNNNNNNNNNNNNNCCCGCCGCCGCTCCCTCAGC
CCACCCGGAGACCCCAGCCCCGAGTCGCCTCCGGATCCCCGGCAGTCTGCGGGAGGTAAGAAGCGGTACTTGCGAAGCTTCGAGCGCCCGGGGA
ACGGCGGAGCGCTGCGGCTAGGCAAGCCCCTGGACGCGGCTCCGCACCCTGCACCCAAATATGCCGGGCTTTTCTGGGGGAGGGGGTAAGGGGG
CAACACACCGCGTCTGCTTCCGAAACAAAACCATCTGGGTTTTTTGTTTTTTTTTTTTTCCTGAAAAGCCTATTCAGCACCCGAAGGCACCCTG
GCTGGAAGAGACCCGCCCTAATCCCTTTTACAGCCCTGGCCCAGGCAGAGAAATAGCTTTAGCGAAAAGAAATTCGCCCGGCTCCACAAGATTT
GTCTGAGTGTGAGGTGACTGGGAACCGGTGCGTCCCCACCCCAAGCTGGGGTTCTCCAAAAGGTGCCGCGGGGAAAGAAGGAAAAGGGGTTAGG
CGAAGCTAGGTGCCTGAGTGATAATGTAGGTCACGGCTGCTCCGGCTTAGGAGACGCGATTCTCGCCGGCGACCCTGCACCATTCCTAGGGATG
GGGTTCAGCCTTTCCCCCCGCCTGTTGAAGAGGTTGAGGTCTCTGAAGGAAGGGGAATGGAAAGGGTATAGGTATAGGCAGCGATGAGTACCAA
GGGTATCTGCCAAGATTGAGGCTCTAGGTCTAGGTGGAGAGGGTTGTATGTAATTGCCTGTTGTTGGTCTGACTGGAGAAAGGCCCTTAATAAT
TAATAAAGTTTCCTGAACCCCGTTAGCGTTTGCGCTCCCGTGATCGCATTTCTGCAAAAGGGGAATGAAAGGTACTTCAATCCCATGCCTGGTA
GCTGGGCTAGAGAGCAAGGACTTCCTTTAAGCAACTAGTGCAAGTGGGGTAGTCGCGCTAGTAAGAGCTGAGATCTGCAGCTTGAACAGCCCCC
CTCCCCCAGGCAGTGCCTTGTGTGAATGAAACGGCAGTTTCCCAAAGTTGCAGAGAGCCACACCACCCCCTGNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNTTCGAATTGGGCTACGGAGATGCTGCTGCCGGAGCCGACCTGTGGGGCAACCCGGGCG
AGGAGGATGCGTTCGGTCTCGGGGGCCTGGGTGGCCTCACTCCTAATCCGGTCATCCTTCAGGACTGCATGTGGAGCGGCTTCTCTGCCCGCGA
GAAGCTAGAGCGCGCAGTGAACGAAAAACTACAGCACGGCCACGGGCCCCCGGGCGTCAGCTCAGCCTGCTCGGCTCCCGGAGTGGGTGCCAGC
AGCCCCGGGGGCCGTGCCCTTGGTGGGTCGTCGAGTGCTAGCCACACCGGGGCCACCCTGCCTACCGACCTCTCCCACCCGGCTGCCGAATGTG
TGGACCCCGCCGTGGTCTTCCCCTTCCCGGTGAACAAGCGAGAGTCGGCGTCGGTGCCCGCTGCCCCCACTAGCGCCCCGGCGACCAGCGCTGC
GGTCACTAGTGTGTCTGTTCCAGCTACTGCCCCGGTGGCTGCTCCTGCTCGTGCAGGCGGCCGTCCTGCCAGCAGTGGGGAGGCCAAGGCCCTC
AGCACCTCCGGAGAGGATACCTTGAGCGACTCAGGTAAAGCCTAGAGTAGAGTTCTTGCTAGCTCCTTACGGACTGGGAAGGTGGGGGTGCTGC
GTTCCCTTTGTTATCATAGATTTGTGCGGGTGATTCTCTTCTGTTCACCAGCCTCCCGGGACAGAAGAGGCTGGAAACACCAGCTACACCTTCT
CTTAAATTTGCCATCCCCTTTTTCGCGAATCCTTGCGGTAGGCATGCCCTCACTGTGGCAAAAGTTGGTGGCCAGCTCAGTCAGTCTGTGGAGT
CCAAAAAACTACCTGTACAGAAATCTGTTAGTCCGTTGGAAAGGAACCTTTAAGCCAGGTCAAGGAAGGGAAAAGACTCCCTGCAAGACGAGGG
CTGCACAATCCCACTTGCAAAAGTTAAGAGAATGCCTGGCAATTCCTCCCTCCCCTCCCCTAGGACTCTGAGCCCTCCGTGGGTGGTGGGCTGT
TTGCGTTTGGTGCGTGGCCAGCAGGCGGCGTTGTGTCTGACGCAAGACAGGCAGGATCAAAAAGACTGGGGGTGATGGGGGCACCCCCTCCCTC
CAGTTCAGCAGCTGGCAGCAAGTGCATTAGTGGTTGTGTACGTTTTCAGAGAGCCTCTTTCCGGTTTTGATTGAGTACTAGTCCCGTTGTGTTT
CCTAGCTGGAATTTCTGACCTAATTGAGCTGAGTTGTGTTCCATAACCCATTGCCCTTGAAGGTTGAGGGCGGGTCCCTACCACTGCCGCTCCT
AAGGAATGCACGCACCCTTAGGATTCATTGGTTCTATTGTAAGCCTTTATCTGCGCTGTCCTCGGAAAGAAAGGGTTAAAAAGAAGAAGAAGAA
AGCTTTTTTTTTCCATGGGGCTGTGAAAGTACCTACCACACTTTACCCAAATCCAGTCTGCCCTTGTCCTTCGAGCTGTAGGTCCAGCCCCCCT
ATTAGACCTTGGAAGCCAGTGTTTTCAAAATGCCAAAGAATAAAAAGTCTAAATTTCCTCCTGGGCTGTGGAGGGATCGCTCTAAGGCTCCTGG
GAGGAAACTTGGTGATAAGCCTGCACTTTGAAAGGGCTCTGTCCCTTTAATGTCTGTGCCTTGACAGCTTTCTGTTAGGAAGCAGTTCCTTCCA
ACAGCTGTCTTCTTGGCTGAAAACCAAAACACTGGCTTAAAGGGACCTACCGGCTGGAGCAGCCTAACATTTCAGCCTTAGAAGAAGAAACCTC
ACAATTGTTCCGCTTTCCGGTCCTCCCTAGATTAAGCAGACGATGTGTTTTGGTTTTCGTGCTTGCTTTTATTTTCTTCTTTTAAGCCATAATT
TTCTCTCCTAGCCTGGCAGTAAACGAAGTCAGAATGAATACACATAGGACGATACAGGCTTTTTTTTTTTTTTTTCTCGTGCACCGCCCCCTTT
GGGACTCTGAGCTACTGTGGATTAGAGCTTTATAATTGAGATCTGATCTCTGCCTAGATGAAAAACTGCTTACTCCGTACAACGCATGTTCGCA
CCAGGAAAGGTTGTTGTCGTAGTGAATGAGATTGTCCTAGAGTTTTAAGGATGGCAGAATCAAACCCAGCCATGGGTTTTACCAAAGGAGGAGC
CTCCGGTCTGAAGGGCGGCTGTGGAAGTGTGTATAAGTAAATCCATGGGGCAGACTGGCTGGTGATTATGCTAAAAGCCCCTATTACCGAGCAT
CTGTCGCCAAGTCTACATAGTGTCTGCCAAACTCAAACACTGTGGTTATTACCCCCATTGAATAGCTGAGGACACTGAAGAGTTTTTCCAGGAT
CATGCCGACAACGGCTCGTTCTGACTCCAGTTGGGTTCCTCGAGTTTCTGCCACATCCTCAAGTTTGTCACCAGGGGTTGAATCCTGGGCCCTT
CCACATCAGACAAGTGCCCTGCTGGCCCCACACCACCCAGGGAAGGAAAGGGGTGGGGCGTACCTCTTGTCCGCGTTGTGATCAGCAAACCTAG
CTGACTTAAATAACAAGTGTATGTTAATCGCACAATTAACCAGAAACTATTTTTCCCCTCAGATGATGAGGATGACGAGGAGGAAGATGAAGAG
GAGGAAATCGATGTGGTCACCGTAGAGAAGAGACGTTCCTCCTCTAACAACAAGGCGGTAACCACTTTCACGATCACTGTGCGTCCCAAGACCT
CCGCTCTGGGTCTGGGGCGAGCACAGCCTGGCGAGCTGATCCTCAAGCGCTGTGTTCCCATCCATCAGCAGCACAACTATGCTGCACCCTCACC
CTACGTGGAGAGCGAGGACGCGCCCCCGCAGAAAAAGATCAAGAGCGAGGCTTCTCCACGCCCCCTCAAAAGTGTTGTTCCAGCAAAAGCGAAG
AGCCTGAGCCCCCGAAACTCAGACTCGGAGGACAGCGAGCGCCGCCGCAACCACAACATCCTGGAGCGTCAGCGCCGGAACGACCTGCGCTCCA
GCTTCCTGACGCTCAGGGACCATGTGCCTGAGCTGGTGAAGAACGAGAAGGCCGCCAAGGTGGTCATCTTGAAAAAGGCCACCGAGTACGTGCA
CGCCCTACAGGCCAACGAGCACCAGCTCCTGCTGGAAAAGGAGAAACTGCAGGCGAGGCAGCAGCAGTTGCTAAAGAAGATCGAACACGCTCGG
ACTTGCTAAACGTTTCCCACACGGACAGTCACTGCCACTTTGCACATTTTGATTTTTTTTTTTTTTAAACAAACATTGTGTTGACATTAAGAAT
GTTGGTTTACTTTCAAATTGGTCCCCTGTCGAGTCTGGATCTGGGTAGGGGGCAGGACACGGGGTTCTGCCATGACCTTGGAAAAAAAACTGAC
TTATGGGATGCTGGGTGGCTTGTTTTCCTCCTCCATATCACCTGGTGACAGCCGTGGAAGTTCGGGACACTAAGGAGCTTCAGGAGGCTGTGAA
GTCACCTTGTTCCGGTCCAAGATTCCAAACAGAGTCATTCCTTCTTTTTACAATGGTGCTTAAGTTCCAGCAAATGCCACAGAAGGGGGGGGTT
GCCATTTGATGCCCCTGGGAACACTTGTGTAAATACCATTGATACACCCGCCTTTTGTATACGTCCTGGGTAATGAGAGGTGGCTCTTGCGGCC
AGTATTAGACTGGAAGTTCACACCTAAGTACTGTAAGAATACCTCAATGTTTGAGGGGCATGTTTTGTATACAAATATATTGTTAATCTGTTAT
GTACTGTACTAATTCCTACACGGCCTGTATACTTTAGTATGACGCTGATACATAACTAAATTTGATACTTATATTTTCGTATGAAAATGAGTTG
TGGAAGTTTTGAGTAGATATTACTTTATCACTTTTTGAACTAAGAAACTTTTGTAAAGAAATTTTACTATATATATATATTCCTTTTTTTCCTA
GCCTGTTTCTTCCTTGTTTACTGTATTTGTTCATGTTTGGTGCATAGAACTGGGTAAAATGGCAAAGTTCTGTGTTTAATTTCTTCAAAATGTA
TATATTTAGTGCTGCACCTTAGAGCACTTTGAAATACCTCATGTTTATGAAAATAAATAGCAATTAAATGATGCAAGGCGATGTTTTCCTTCCT
AGCTTGGCTGCAGTCTCTGCACACAGTCCATTCCTTACCCGGACTTTTAACATTGTGGAATCCTGGTTAGGGAGGGTACTTGTAGAGGGATAAA
TGGGTTTGTGAATTCCACAGATTAGAACCAACTACTTTCAATAGGCAATCAATGGGTGTTAAAGCTCTTTCAAATATTTTGTTTTGGGTGTGAG
TGTGCCCACAATGTCACATCCCCTGCAACTGGAGTTCAAGTCTAGGAACAAGGCAGAGGCCCTGCGCAACAGTAACAAGTGACTCACCCCTGAG
CCATCTGTCACCCCTTCATTTTTGAGGCCTCTTTGGGTCTCTAGCTGGGATTATAGGTGTGTGCCCCCCATATCTAGTTACCAAAACACTTTCA
CATACTCGAACTCTCAAAGCATTTTAGATAGTGTAGATACCAGCCCAAAGTTTGGAATAAGCAGTAAAATTGGCAAGTTGTGCCGAAATTTACA
TAAACACTTGTCCTAGTTGTTTGGAAGGACTTGTTCCTGAAGATTCCCCCAGGAAGAGCTAGGTGTGGAAGCATATCTGATGCCTGCAGAGGTG
ACAAGAAAAATGTCACCTAGCCCTCTTAGCACAGCAAGCTTCTATATTGCAAGCCCAAGGCCGCAGAGTAACACTTAGTCCAAAAGAAATCTAG
GACCAGTATAATATTCTGGACTGAGCACTAGTGAAATAAGAGGATGGAGTCACTGTTCATCATTGATCTCGGTCACCAGCCTCAGCTCCAGTTC
CCAGAGTACTGAAGACCAAGAAGGGGAAGGAGAGCTGTCATTGGGTGGACTGTGGGGGTGCCTCAAGGAAGAATGAGCTACAGACCTTGGAGAA
GGCCAGAACTAAACATGAAGGAGGCTTCCCTCTATCTACTCAGGGAAAATTTTCTGGAAGTCTGAAGACCATCTACCTTACTTAAGAACAGGAC
TTCTATTTTTCTTTGGGGTAACAGACTACACACAGCATTTCCCTATGGTTTCATTCACTGCAGGTTTTTAACTTCAAGTTGAAAAAAAAAATGT
CTTCATGGAAAAATGCAGCCCAAGGGTCCAGACAGCTTTGCTTGTGTGGCTGTACTGTCAGGGACAGAGATCAGGCTGCGCAGGTGGTTGGCCA
GACAGGTAGAAGGTGAGCCATGGAGGTCCTTATTTGGGGTGACAGCTACAGATGCTGTCTTAACAGTTGCTGTTGGGTTGGTGGTACTGCCTGC
ATTCTCTGGTGATTTGACAAAGGTTGGAAAGTGGGCAAAATGCCCTTTACAAAATCCTATAACTCCCCAGTCCCAAACTAGCAAATAATAAGCC
ACTCTGAAGGTCCCATTCAAAAGAACTCATCTGTTGCATCTTGCAAGTCCTACGTAACAAGCCCATCACTGTATCAGTACATCTAGAAACCAAA
TGGAACTTAAAGATCTCAGGTTAATTGAGCACCTAAGAGTGTTGAAGCCTGCCGCAGTCAACTGAATTGCTCTGTTTCTCTTCTCCCAAAGGGG
ACCTGAAGTGTGACAGATGAGCTAGAATTCTACTGTGTAAGAAGAAATAGAACAGAGACCACTGAACTGAAATTTCTACCACATTCTTATTTTG
GTGACTACGGGGAAACTTGTAGATAATGCTCTCTTACATCTCCAGGACCCACTTCTGCCTCGAAAGCTCTGTTACTCTCTTGACATCAATCACA
TAACAACAACAAACTCTCTGCATTCGGTGGATCATAAATAAAAGAACATATATTGCTCTTCCAGTGGATCTGAGTTCAATTCCCAGCACCCAAG
TTAGGTGGCTAAGCTGCTTGTAACTAAGGCACCAGGGATTTGACATCTCTAGACTCCTGTGACTTACACTCGCATAGATACACACACACAATTT
AAATAAATACATTTAAAATAAATAAAAACACACTAAAAACTGTTAACACTTAGGTTAAGACACACTTTTGTTTCCTCATCTAGCTGGAGGAAAG
TGTTCCTACTGTAACTCTGCAGTTCTCTGTACAATAATTGGAAAAGAGAAGAATCCAAGCAGTTGTCTGAAAAACACAAGACTGAGATACTCAC
AGAAAGAAACTCTTTCTTAAAAGAGTAAACTGGCACTTCTGTGGAGTGTTCCACCATCTCTCAATCACTTCCTCTCACAGAATGCAGGTGGGGT
GTCCCTGGACTAACTTGGGGCACAGGACTGACATTGAGGCCTCGTGATCAGCCTGGTGGTATCTCGCAGGACTCCTGTAACCTATAGGCTGGCC
TCCACCTCAATATGTAGCTGAGGATGGTCTGAGCTCCTGGTCCTCCGACCTCCACATTCTGGATACTGGCTAACAAAGTGAGCCAATATACCTA
TGCATGTATTATGTAAGTGCTGGGGATAGAATCCAGGGTTTTGTGCATGATAAGCAAGCACTTCCTGAGCTATGTCACGTACCCTTGCTGATGG
AATTCCCTGGCTCTTTCCTCTCAACCAATGTTATTAACATAGGAACTAACATTTGTCAGGTGTTTCTGGTGCTTGGGAGAAATCAATGAAGAAC
AGCAACAACAACAAAAAAAAAACAAAATTGCCAACTGCACCCAATTCTTGTCTCCTTGTGGTTTATAATCTAGGAGACAATGGGAGTAAGAATA
TAAATAAGCAAAGCAAGCCAACAAATAGCACCAATGAGGTAGTGAGTAATTTATAAAAGAAGTGGACTGGTATAGAGGAATTCAAGATTGCCAC
CAGAGGGGATGGGGAGCCAGGCCAGTGCAGATGCACCTGGTCCTTGGACCTGTGGAGGAAAACGCAAGGTGACTGACAGGGCTGCTCAGCCACC
AGAGCAAGCTGCTCAGGTTCTCCATTTAGTCCGTCTCCCCTCCTCACACCCTGGACATGCGTGTTCCCTACTATGTCCTTTGCCTATCTGCTGA
AAGCGAAGCATCAAACTATGATGCAGCTGCAGTCTGATTCCCTGGGACTGAGTCCCACAGTTAACACAGTTCCTCATATTGTGGTGACCCCCAG
CCATAAAAGCATTTTTGTTGCTACTTCATAACTATGATTTTGCTACTGTTATGAATCGTAGTGTAAATATCTGATATGCAAGATATCTGATATG
AAACCCCAGTGAAAGGGCTGTTCAACCCCACAACTGTTTCCACCCACAGACTGAGAGTCTCTGCTACAGAGAAAGGTGTGTGTGTGTGGGGGGG
GGGGGGTTATTTGGTATCGACAGAATTGTTCCTTTGTTTCCTCTGTATTTTTCTAGGAAAAAAGGACTATTTAGAAAAATGAGCAGTAAAGCAA
ATATATAAGCCTGACCTCAATGAGAACTCTGCAGACTTTTGTTACCCCCAAAAGGCTTTATAAAACCTAAAAAACGGGCGCTTTGGAAAAGAGT
AAAGGTAAGCTCTGAAGCCTATTTTAAGACAGAGACAGATCCTAGGAACCTGAGTTCCACGGACCATCTATGCTTTCTACAATGGTTTATTAGT
TCTCCTTACTTGTAAAGGAAATCCCTTCATAGGCGACTGACCAAGGCCACCTGATCTCTCTTGATACTGACAGGTCATACTACAAGACATCCAT
CCTGAAACCTTTGACCAGAAGTAACAGAGCCTTGTTTATAGTGGAAGCATGGCGACTGGCTGGGACAGTGCATATTTGGGGAACACACACACAC
ACACACAGCCTAGGCTGCTTAGATGTGAAACGTAGGTGATTGGCGGGAGCATGTGTGCACTGAGCGGCAGCGTCCAGTGGACTTTCCTCTGCTA
TCTAGCAGGCCAGAGATTTTTTTCAGGCCTTAAGGGCTAACAGATCTTTTTTTAAGGATTATTTTTATTTTTATTCCTGTGTGTGTCTGTGTAC
CCAAATGTACCTTCAGATGCCAGAAGAAGGTATCAGATCTCCAGCAACTGAGTTAGAGGTAGTTGTGAGCCACTCAGTGTGGGGGTTGGGAATC
AAATCCAGGTCCCCAGGAAGAGCAACAAAGGCTCTTAACTGTTGAGCCCTCCATTTATGCCATAATCTTTTTAAGTTATTCATTCATTTATTCA
TTCGTCACTCAGTGGGGGGGGGGGGGGTGCATGCCACAGTGCATGTAGGTATAGAGGTCAGAGGGACAACCTGAGGGAGTCAGCTCTTTATTTC
CAGACCAGCTGGGCCTTAGGAGAGATCTTTGGCCATCAGGCTCAAGGGCAAGAGCCTGTTTTTTTTTTTTTTCGATGAGCCAACCATGTCAATG
GCCCCTGTTAGAGAATCTTTGAGAGGCAGGAAAGCAAGTAGAGGTCTTCTCTAAAGTTTTTCTGAAGTGAGAAAGAGAGTCAGAGATAGACACA
CACACACACACACACACACACACACACGGAGAGACAGAGAGACAGAGACAGAGATTCAATACTGAGAGTTGAACCCAGACTTTCACACATGCTG
GACAAGTGTTGACTACCACTTAGCTACACCCCAAGCTCTCCTTTTGCCTTGTATTTTGAGACAGGGTCTTACCAAGTTGTCAGGCTGGGCTTGC
ACTCCCTCTGTAGCTCAGATAGTCCTTGACCATGTTGTCCCCCTGTCTCAGGCCCTGTATAGCTGGGGTTGCAGACCTGACCCACCAGGTCTGT
GTATTTCGTGTGTCTTTGTTTTGTGTCTTAGGCTCCCTTTCATGAGATGATAGGACGAGGTGACACTGAGCGTTGCCTGGGAGACAGACTTTGT
GGGCCAGCAACCAGCCTTGANNNNNNNNNNNNNNNNNNNNACCTCCCTGTTGGCTGTGAATATGTTTCCACTGACACCTTTGGGAGGTGATTTC
TCCAGGAGTGCGACCTCACTTGGTAATTCGCCTGGGTAACAAAGGTACCAGGCCTGTCTGTCACTGTTGCCCAACTTCCCACTGACCCTCCGGA
GTGGAGGCTGCTGCTGCCCTGTACCCTCACCACCCCACCGTCAGGTGCATAGCTGCTGATTCTGGTTGAGCAACCCCAGAGTATTGTTTCTTGG
GTATTGTGATGAATCACAAAGCAAAACAAGGCTGGGGAAAGGAAGTAGTACTTAGTCCAAGTTCAAAGTAAGCTGCTTTCATTGGAGGCAGTGA
CTACAGAGAGCAGGCTAACTGAGGTAGAAAACACCAGACAGCGCTTGGATGACTTAACAGTTAAGAGCACAGACTGCTCTTGTAGAGGAGCCAA
GTATGGCTCCCAGCACACACACTCAAGGGGATGGCTCACGACTGCCTAAAACTTCAGTTCCAGGGGGCTGACGCCCTCTTCTGGCTTTCATAGG
CATCTACATTCAAGTGCACACACACACACACACTTAAAAATAAAATAGAACTCTTAAGCCAGGGCTACACAGAGAAACCCTGTCTCGAAAAACA
AAACAAAACAAAACAAAACAAAACAAATCTCTTTTACTGGTGTTCTTTTTCCTTCTATTATCCAGGGTGACTGACCAACCCATCTTGGCTTTCA
AACTAAAGGACCTGGATCCTAGGGAACATTGAGGTCCCCTTACTCTGCCCTGCCACTCCTCCCCCCCCCCCCCCCCGACAGTGTTTGCTTCTTC
ATTGTGCATCATAAATTGTACAGCCACCTTCTGCAGGTCAACCAACTCTGCCTTCTTTGAGGGTCAGCCAAATGATAGGGAAAGGAGTGATTGG
CCCACGCCAGGCCAGACTGGGAGGAACATGGACTTGGGGTTGGATAGCCCATAGATAAGAGATCTGGAAACCTTCCAAGGGTAATAAAAGGAGA
GAAATCTATTTTCCTTTTCAGAGACATTGAATGTGAAGCTGGTGGGTCTCACATAAATGTCAGGGACCAATTTAANNNNNNNNNNNNNNNNNNN
NTTAGGCTCCCTTTCATGAGATGATAGGACGAGGTGACACTGAGCGTTGCCTGGGAGACAGACTTTGTGGGCCAGCAACCAGCCTTGACTCCTC
TCGCTTGCTCACCTTCTTACTCTCCGGCCAGGAAAGTTTGTATCAGTTCCCCATCTGTGAGCAGAACAGGCTGGAGCTGGTGAGCTCAGAGACA
GGTGCAGCTTGCAGTCACTCATCCTGAGGAAGGGCTGCAGCCTTCAGCATCCCCACAAGATGCCAAGCACGGTGACACACGCTTTTAATCCCAG
CACTGAGAAGGGAAAGCCTAGCCTGGTCTACATAGCAAGTTTCAGGATAGCTAGGGCTACATATAGAGAACCTGTCTTAAAAATAAAAGATGAC
TAAAAGAGCTAGGGATGTAGATCAAGTGGTAATTTGTCTGGAGTGCAGCAAATATAAACGCCATATTGTGGTGCACTCAGGAAATCCTACATGT
AGGCAGAGGATCAGGCGTTCAAGGCCATCCTTGCCTACACAGCAAAATGAAGTCAGCCTGGACTATATATTTCAAAACAAAAACAAACAAAAAC
CAACTCAGATGTCGTCAGTGCTGGTAAACAGTGGCACTGTGGTTGGAAACTTGGGAACCTATCAGCTCTCTGCTGGTCACCTTCTCCAGAACTG
TCGTCATTCAGTGCATCCATCCCTCTGATGCACGCTGTCTTCCAGCCTAAAAGCGGGAGCTGGGGATGTTTTTCATCTGTGGAGTGTGGAAGCT
AGCTTGCAGGAGGGCCTGGGTTCAATCCCCAGTGTTGCAAATGTAAAATAAGAATCACAGCTGCAAGGTTCTTGCAAGGATCAAGTCATAAACC
ACTGGAGGGATGTGACAAACTTTGGAAACATGAGCGCCATCCATGAGGTCTTACTGCTGCCCGGACTCAGACCACACTTGAGCAGCCTCCCTCC
AGTCTACAGACTGCCATCTAGATTCGACCCTCAGGGTGTTTCCCCAGGCAACAAACTCCTTTCCTTGTGTAGCAGGCTTCCTCTGGTTGATCCC
TTGTACCTAGAATAGGGCTTTGCTTGGCAGTCTAGATGTTATTTTCATAGGGAAAACCTGGCTATTTTCTTCAAGCCAGCTAGAGCCATGATTC
CTGCAAACGAACTGGGTACTCTGGCTGGTCTCTGCTGAAACTAGAGAGAAGTATGATTAATCCATTGTTGAAATTGGTTACTCAAATGAAAACA
GAACATGGTAACAGACATTTCATACCTTGAGTCTTTGCTTGGGTTATGCTAGTCACTTCTTTTTGGCACAAAACAAGAAAGAACAAGGGCTTTA
AACCAAGAGTAGATACAAGTTTGCTCTTTTCTTTTTTCCAGAGCAACTGAGCTATAGTAAAGAAAAAGAAACCCCCCAAAGAGTGGGAAAGCAC
GAAGGAAAGAACACCCCAGGTTTAACTGCATGGGTTGAAAAAAAATTCTTTATCACCTGGCATGGTGGTGCATCTCTACATTCCCAGCACTCGG
AAGAAGCAGAGGATTGTTCAGACTGACTGGTCCAGGTACGGTCTAGGCCAGGTGGGGCTACTATATACTGAGACTTTGTCTCCAACAAAACAAA
ATAAGAAAGGAAATCTTTCCCACCCTGTGGTGGGTCCTACTTCTGAATTTTCTTTCTTTCTTTCTTTCTTTCTTTCTTTCTTTCTTTCTTTCTT
TCTTGCTTGCTTGCTTGCTTGCTTGCTTGCTTGCTTTCTTGCTTCCATCCTTCCATCCTTCCTTCCTTCCTGTCTNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNTCCTTTTTTTTTTTTTTTTTTTAATTTGTTTTTTTTGACAAGGCCTTGCTATGTAGCCCCAACTAGCTT
AGTACTATGTAAACAAGGCTGGTTGGAACTCACAGCGATTGTTCTACATCAGCTTCCCAAAGGGCTGGAATTACAGGCATGAGCCACCACCTTC
TATTTTATTGCTCATGTTCTGCCCGCCTCCCTCCACCCTCCTTCTTTGGACAGGGTTTTTACTCAGCAGCCCTGGCTAGCTCAAAGAGACACAC
ATGTCTTTGCCTCCATGTTTTGGAATGAGAGGCTTATGGAGGCACACTCAGCCTTCTGCTAGCATCCTCTAGCTTCCTCTAGTCTCCTCCTTCT
TTCAGTCTTTGTACGCTGCCAAAAGAAACGGTTGTCCGATGAAAGAGCACAAAGCAAGCTGAAAGGAGGTGAGGGAAACACGTAAAATAAAATG
CTCCCCCAGTTCCTAGGGAAAATTGAGCCCTACTTAGTAAGAAAAGCTGCAGATTCAGGTACCTGGATGTGGGTCAGGACCTTGATGCTCTAGG
ATTATACCATCTCCCAGGAATCTTCCTGGGGCTTCCTGTGGTGGCTTTCCACGGTCTCAGGATGAGACCACCTGGGCATAAGCCAGTTGTGGGT
GGAGGGGCTGCATTCCTCAGAACTTGAAGATGGATCCCTTCACAAAAAGAAGAGGATGGAAGAAAGTAGACCTTCCAGGAGCCTTTAGCATGGG
TATTCACATGGATTTAACTTGGGGACAAACCTGCGGTTAACTTGGTTAGCAACTGCTTGTAAATGGGAGAATCCTTATTCCAGGGGCTGGTGCA
CATGGGGGAAAAAGACAGAGCAGTCCATGGCCTCCTGAAGGGAATGGTTCTATCATGTTAATGCTCTGCATCTTATTACAGTCAGGACAAAGGG
CCTGGAGTTTGGGACTGAGAGTTTGTTTTGGAGTCTTTTAATATGGAGATGACTCTCCCTAAGGGGAAGGAAGAGGACCCTGGAGTCAACCCAC
AAACTTGTCTTGGCATGGTGGTGGTGGGGTGTTGAGACTCACCACAGGTGCGGTGCCCCNNNNNNNNNNNNNNNNNNNNAAATGGAAACAAGGA
CTTTGGTGCTACTGATGTCTGTCTCTCTGCCTATCTCTTGGTGTT
MOUSE SEQUENCE - mRNA
GAATTCAGTGGGAGGTCCCGGTTTGGAAGAGGCTAGGGGTGTGAAAATGAGGGTGTTAAAGGTTTTTGTAAAGGTAATGGGTTTTATACATATG
TGAGCCCAATAAGAAAGGCCTTGGAAGGGTGGCTACCCTCATTCACGGCTACTCCATAGAACCTGCCGTAGTCCCCANTATGTAGGCATTCGGG
GATAGATGGGAAGACTTTCACACATGCTAATACACATTTTGACCTTGACTACACAGGCATTACACTATTACTTACTGGAGGTGAATGAGGGGTG
TATTTAATCCAGAGGGAAATGGAGACGCTCAACATAAATAAGCTTAAGGGTTTAAGGATAAATCCCACCCTACTGAGAATGTGTGTTGAAAAAG
GGCTTCACCTAGGGACCTAGGTTACTCTTGGAAAGTTTTGTGGGGCAAGAGCATTTATTTTTAGCCCCACATTTAGGAAACAGACCTTTTAGCA
TGCTTGCCTAGACAGAAGTGAGACGCTGTGGGATGGGGAGGGAGGGAAATGTGCCCACGGCTGTTGAGGAAAGAATACACGCACCCTGCTCTAG
TCTAAATAAGATGTCGCGCTCTCTCTGTTCACGCCACCTCGTAAGTGAGGCAAATGGGACACAAAGAGGCCAAATTAATGACGGCTTTCCTTGA
TGTCACCATCCGTAGAACTCGAGGTATACAGAGCAGTCAGTGATTTGGGCAAGGAAAGCAGCGGTCCGTAATTCAACCTTGGCTTCGGAAAGGC
TTTTGTTGGGAACGGTGGGCGCGACTTTAGGGGACGTCCCTCCACGCGTTTCACAATCCCTTCCGCGGTAGCAAGGATTCGCTAATTGTCTCGC
GGGGGTGGCCCCACTCGGGCTCCGAGGCTGGGCGGTGCGGCTCCGCTTTCTGCTTGGTCTCCGCGAGGTGTCGCCTTCGGCGAAGAAAGCGGTA
AGGCGCCACCCTCGTAGCCCGCACTTATTTATTTTCAAACAAGGGGGGCGCCCCTTCCTTTCAATTTGAAACTAGAAACCTCTGCGGTCTCGTT
TCTACGTGGGCGCGTCCTCGGCCTCCCATTTTGCACTCCTGTGTCGCCTTCCTTCGAGATCACACACGAACAGCCCCTCGACCAGGCCTTGTTC
CACTTTAGCCTAGAGCTCGTCCCCACACAACACACACTCGCGCGCAGTCCACGCAGGGACTGGGCCTAGAAACCTCTTTTCCCATTCCCAGAAC
ACACCCAGCGACCTTTGACCTTTAAAAAAAAAAAAACACGGAGTGCAGCGGGTGCAAGCCAGGGGGTCCAAAAGGGAGGGGGAAGCGCTCCTCG
GGTTTCCCAGCTTTGCTGCTTGCAGTCTGAAAAACGAGAGGGGGGAGGGGGAGAGAGGGAGGGACGGAGGGGGAGAGGAGAAGAGGGGAGGAGG
GAGGGGAGAGAGAGAGAGAGAGAGAGAGAGAGAGAGAGTCGGAAAAAAAAGAGAAAAGCAAATGGCTTTTGGCGCGAAAGGCTTTGGCGCCTCC
CCTGATTTTTATGGAAATCAAGAGGGCGGGGAAAAGCCTCTTGCCTCTGCCTTTCTCCCTCCCCCTCGTCTGCGCCACAGCCCCCTTCTCCCCC
GCCCCCCGGGTGTGTCAGATTTTTTCAGTTAATAATATCCCCCGAGCTTCAAAGCGCAGCCAGTGACAGTCATCTGTCTGGACGCGCTGGGTGG
ATGCGGGGGGCTCCTGGGAATAAGCGCGCACACACTGCAGCCGCCGGAGGACAACCCCCTCCCGCCGCCGCTCCCTCAGCCCACCCGGAGACCC
CAGCCCCGAGCCCCTCCGGATCCCCGGCAGTCTGCGGGAGGTAAGAAGCGGTACTTGCGAAGCCCGAGCGCCCGGGGAACGGCGGAGGCCCTGC
GGCTAGGCAAGCCCTTGGACGCGGCTCCGCACCCTGCACCCAAATATGCCGGGCTTTTCTGGGGGAGGGGGTAAGGGGGCAACACACCGCGTCT
GCTTCCGAAACAAAACCATCTGGGTTGGGTTTTTGTTTTTTTTTTCCTGAAAAGCCTATTCAGCACCCGAAGGCACCCTAGCTGGAAGAGACCC
GCCCTAATCCCTTTTACAGCCCTAGCCCAACAGAGAAATAGCTTTAGCGAAAAGAAATTCGCCCGGCTCCACAAGATTTGTCTGAGTGTGAGGT
GACTGGGAACCGGTGCGTCCCCACCCCAAGCTGGGGTTCTCCAAAAGGTGCCGCGGGGAAAGAAGGAAAAGGGGTTAGGCGAAGCTTAGGTAGG
TGCCTGAGTGATAATGTAGGTCACGGCTGCTCCGGCTTGAGAGACGCGANNNNNNNNNNNNNNNNNNNNTCTCCAAAGGTTGAGGTCTCTGAAG
GAAGGGGAATGGAAAGGGTATAGGTACAGGCAGTGATGAGTACCAAGGGTATCTGCCAAGACTGAGGCTCTAGGTCTAGGTGGAGAGGGTTGAT
AGTAATTGCCTGTTGTTGGTCTGACTGGAGAAAGGCCCTTAATAATTAATAAAATTTCCTGAACCCCGTTAGCGTTTGCGCTCCCGTGATCGCA
TTTCTGCAAAAGGGGAATGAAAGGTACTTCAATCCCATGCCTAGTAGCTGGGCTAGAGAGCAAGGACTTCCTTTAAGCAACTAGTGCAAGTGGG
GTAGTCGCGCTAGTAAGAGCTGAGATCTGCAGCTTGAATAGCCCCCCCTCCCCTAGGCAGTGCCTTGTGTGAATGAAACGGCAGTTTCCAAAGT
TGCAGAGAGCCACACCACCCCCTGCATCTGCAAGCCCCCTCCCACTCCCAGTCCTAGACAGCTTGTACACAAAAGGAGGGAGTAGGGGAGACGC
TCAACTTTCTCCCACCTTCCAGAGCTGTGGGGAGCTTGCAGAAGAGATTGGGGGCTCCCACTGCCTGTCCCCACCAACCCACCCCTTTGGCTCA
TTCTCTCTTGGTTTGCCTATTGGTTGTAGAGTTGGAGGTTGGCGCGACTCTGCTGCTCTCCACGGGGAGGAAGCACTCCCCCATATTAAAAAGA
GCGGAGATATTAAAAAGAGAGGCGAACCCATGCCCAGCTGCACCGCGTCCACCATGCCGGGGATGATCTGCAAGAACCCAGACCTCGAGTTTGA
CTCACTGCAGCCCTGCCTCCACCCGGACGAAGATGACTTCCACTTCGGCGGTCCCGACTCGACCCCACCGGGGGAGGACATCTGGAAGAAGTTT
GAGCTGCTGCCCACGCCCCCGTTGTCGCCCAGCCGCGCCTTCCCAGAGCACAGCCCGGAGCCTTCGAATTGGGCTACGGAGATGCTGCTGCCGG
AGGCCGACCTGTGGGGCAACCCGGCCGAGGAGGATGCGTTCGGTCTCGGGGGCCTGGGTGGCCTCACTCCTAATCCGGTCATCCTTCAGGACTG
CATGTGGAGCGGCTTCTCTGCCCGCGAGAAGCTAGAGCGCGCAGTGAACGAAAAACTACAGCACGGCCACGGGCCCCCGGGCGTCAGCTCAGCC
TGCTCGGCTCCCGGAGTGGGTGCCAGCAGCCCCGGGGGCCGTGCCCTTGGTGGGTCGTCGAGTGCTAGCCACACCGGGGCCACCCTGCCTACCG
ACCTCTCCCACCCGGCTGCCGAATGTGTGGACCCCGCCGTGGTCTTCCCCTTCCCGGTGAACAAGCGAGAGTCGGCGTCGGTGCCCGCTGCCCC
CACTAGCGCCCCGGCGACCAGCGCTGCGGTCACTAGTGTGTCTGTTCCAGCTACTGCCCCGGTGGCTGCTCCTGCTCGTGCAGGCGGCCGTCCT
GCCAGCAGTGGGGGGACCAAGGCCCTCAGCACCTCCGGAGAGGATACCTTGAGCGACTCAGGTAAAGCCTAGAGTAGAGTTCTTGCTAGCTCCT
TACGGACTGGGAAGGTGGGGGTGCTGCGTTCCCTTTGTTATCATAGATTTGTGCGGGTGATTCTCTTCAGTTCACCAGCCTCCCGGGACAGAAG
AGGCTGGAAACACCAGCTACACCTTCTCTTAAATTTGCCATCCCCTTTTTCGCGAATCCTTGCGGTAGGCATGCCCTCACTGTGGCAAAAGTTG
GTGGCCAGCTCAGTCAGTCTGTGGAGTCCAAAAAACTACCTGTACGGAAATCTGTTAGTCCGTTGGAAAGGAACCTTTAAGCCANGTCAAGGAA
GGGAAAAGACTCCCTGCAAGCGAGGGCTGCAGAATCCCACTTGCAAAAGTTAAGAGAATGCCTTGCAATTCCTCCCTCCCCTCCCCTTGGTTCA
GCAGCTGTCAGCAAGTGCATTAGTGGTTGTGTACGTTTTCAGAGAGCCTCTTTCCGGTTTTGATTGAGTACTAGTCCCGTTGTGTTTCCTAGCT
GGAATTTCTGACCTAATTGAGCTGAGTTGTGTTCCATAACCCATTGCCCTTGAAGGTCGAGGGCGGGTCCCTACCACTGCCGCTCCTAAGGAAT
GCACGCACCCTTAGGATTCATTGGTTCTATTGTAAGCCTTTATCTGCGCTGTCCTCGGAAGAAAGGGTTAAAAAGAAGAAGAAGAAAGCTTTTT
TTTTCCATGGCCATGGGGCTGTGAAAGTGCCTACCACACTTTACCCAAATCCAGTCTGCCCTTGTCCTTCGAGCTGTAGGTCCAGCCCCCCTAT
TAGACCTTGGAAGCCAGTGTTTTCAAAATGCCAAAGAATAAAAAGTCTAAATTTCCTCCTGGACTGTGGAGGGATCGCTCAAGGCTCCTNGGAG
GAAACTTGGTGATAAGCCTGCACTTTGAAAGGGCTCTGTCCCTTTAATGTCTGTGCCTTGACAGCTTTCTGTTAGGAAGCAGTTCCTTCCGGCA
GCTGTCTTCTTGGCTGAAAACCAAAACACTGGCTTAAAGGGACCTACCGGCTGGAGCAGCCTAACATTTCATTCAGCCTTAGAAGAAGAAACCT
CACAATTGTTCCGCTTTCCGGTCCTCCCTAGATTAAGCAGACGATGTGTTTTGGTTTTGCTGCTTGCTTTTATTTTCTTCTTTTAAGCCATAAT
TTTCTCTCCTAGCCTAGCAGTAAACGAAGTCAGAATGAATACACATAGGACGATACAGGCTTTTTTTTTTTTTTTTCTCGTGCACCGCCCCCTT
TGGGACTCTGAGCTACTGTGGATTAGAGCTTTGTGGTTGAGATCTGGTCTCTGCCTAGATGAAAACTGCTTACTCCGTACAACGCATGTTCGCA
CGAGGAAAGGTTGTTGTCGTAGTGAATGAGATTGTCCTAGAGTTTTAAGGATGGCAGAATCAAACCCAGCCATGGGTTTTACCAAAGGAGGACC
TCCGGTCTGAAGGGCGGCTGTGGAAGTGTGTATAAGTAAATCCATGGGGCAGACTGGCTGGTGATTATGCTAAAAGCCCCTATTACCGAGCATC
TGTCGCCAAGTCTACATAGTGTCTGCCAAACTCAAACACTGTGGTTATTACCCCCATTTAATAGCTGAGGACACTGAAGAGTTTTTCCAGGATC
ATCCGACAACGGCTCGTTCTGACTCCAGTTGGGTTCCTCGATCGAGTTTCTGCCACATCCTCAAGTTTGTCACCAGGGGTTGAATCCTGGGCCC
TTCCACATCAGACAAGTGCCCTGCTGGCCCCACACCACCCAGGGAAGGAAAGGGGTGGGGCGTACCTCTTGTCCGCGTTGTGATCAGCAAACCT
AGCTGACTTAAATAACAAGTGTATGTTAATCGCACAATTAACCAGAAACTATTTTTCCCCTCAGATGATGAGGATGACGAGGAGGAAGATGAAG
AGGAGGAAATCGATGTGGTCACCGTAGAGAAGAGACGTTCCTCCTCTAACAACAAGGCTGTAACCACTTTCACGATCACTGTGCGTCCCAAGAC
CTCCGCTCTGGGTCTGGGGCGAGCACAGCTTGGCGAGCTGATCCTCAAGCGCTGTGTTCCCATCCATCAGCAGCACAACTATGCTGCACCCTCA
CCCTACGTGGAGAGCGAGGACGCGCCCCCGCAGAAAAAGATCAAGAGCGAGGCTTCTCCACGCCCCCTCAAAAGTGTTGTTCCAGCAAAAGCGA
AGAGCCTGAGCCCCCGAAACTCAGACTCGGAGGACAGCGAGCGCCGCCGCAACCACAACATCCTGGAGCGTCAGCGCCGGAACGACCTGCGCTC
CAGCTTCCTGACGCTCAGGGACCATGTGCCTGAGCTGGTGAAGAACGAGAAGGCCGCCAAGGTGGTCATCTTGAAAAAGGCCACCGAGTACGTG
CACGCCCTACAGGCCAACGAGCACCAGCTCCTGCTGGAAAAGGAGAAACTGCAGGCGAGGCAGCAGCAGTTGCTAAAGAAGATCGAACACGCTC
GGACTTGCTAAACGTTTCCCACACGGACAGTCACTGCCACTTTGCACATTTTGATTTTTTTTTTTTTAAACAAACATTGTGTTGACATTAAGAA
TGTTGGTTTACTTTCAAATTGGTCCCCTGTCGAGTCTGGATCTGGGTAGGGGGCAGGACACGGGGTTCTGCCATGACCTTGGAAAAAAAACTGA
CTCACGGGATGCTGGGTGGCTTGTTTTCCTCCTCCATATCACCTGGTGACAGCCGTGGAAGTTCGGGACACTAAGGAGCTTCAGGAGGCTGTGA
AGTCACCTTGTTCCGGTCCAAGATTCCAAACAGAGTCATTCCTTCTTTTTACAATGGTGCTTAAGTTCCAGCAAATGCCACAGAAGGGGGGGTT
GCCATTTGATGCCCTTGGGAACACTTGTGAAATACCATTGATACACCCGCCTTTGTATACGTCTGGGGTAATGAGAGGTGGCTCTTGCGGCCAG
TATTAGACTGGAGTTCACACCTAAGTACTGTAAGAATACCTCAATGTTTGAGGGGGATGTTTTGTATACAAAATATATTGTTAATCTGTTATGT
ACTGTACTAATTCCTCACGGCCTGTATACTTTTGTATGCGCTGATACATAACTAAATTTGATACTTATATTTTCGTATGAAATTGAGTTGTGGA
AGTTTTGAGGTAGATATTACTTTACACTTTTTAACTATAAACTTTTGTAATAAATTTTACTATATATATATATTCCTTTTTTTCCTAGCCTGTT
TCTTCCTTGTTTACTGTATTTGTTCATGTTTGGTGACATTAACTGTGTAAAATGTCAAAGTTCTGTGTTTAATTTCTTCAAAATGTATATATTT
AGTGCTGCACCTTATGAGCACTTTGAATACCTCATGTTTATAAAATAAATAGCAATTAAATAATGCAAGGTTGATGTTTTCCTTCCTAGTTTGG
CTGCAGTCTCTGCACACAGTCCATTCCTTACCCGGACTTTTAACATTGTGGAATCCTAGTTGGTGAGGGTACTTGTGGAAGGATAGATGGGTTT
GTGAATTC
MOUSE SEQUENCE - CODING
ATGCCCAGCTGCACCGCGTCCACCATGCCGGGGATGATCTGCAAGAACCCAGACCTCGAGTTTGACTCACTGCAGCCCTGCTTCTACCCGGACG
AAGATGACTTCTACTTCGGCGGTCCCGACTCGACCCCACCGGGGGAGGACATCTGGAAGAAGTTTGAGCTGCTGCCCACGCCCCCGTTGTCGCC
CAGCCGCGCCTTCCCAGAGCACAGCCCGGAGCCTTCGAATTGGGCTACGGAGATGCTGCTGCCGGAGGCCGACCTGTGGGGCAACCCGGCCGAG
GAGGATGCGTTCGGTCTCGGGGGCCTGGGTGGCCTCACTCCTAATCCGGTCATCCTTCAGGACTGCATGTGGAGCGGCTTCTCTGCCCGTGAGA
AGCTAGAGCGCGCAGTGAACGAAAAACTACAGCACGGCCACGGGCCCCCGGGCGTCAGCTCAGCCTGCTCGGCTCCCGGAGTGGGTGCCAGCAG
CCCCGGGGGCCGTGCCCTTGGTGGGTCGTCGAGTGCTAGCCACACCGGGGCCACCCTGCCTACCGACCTCTCCCACCCGGCTGCCGAATGTGTG
GACCCCGCCGTGGTCTTCCCCTTCCCGGTGAACAAGCGAGAGTCGGCGTCGGTGCCCGCTGCCCCCACTAGCGCCCCGGCGACCAGCGCTGCGG
TCACTAGTGTGTCTGTTCCAGCTACTGCCCCGGTGGCTGCTCCTGCTCGTGCAGGCGGCCGTCCTGCCAGCAGTGGGGAGGCCAAGGCCCTCAG
CACCTCCGGAGAGGATACCTTGAGCGACTCAGATGATGAGGATGACGAGGAGGAAGATGAAGAGGAGGAAATCGATGTGGTCACCGTAGAGAAG
AGACGTTCCTCCTCTAACAACAAGGCGGTAACCACTTTCACGATCACTGTGCGTCCCAAGACCTCCGCTCTGGGTCTGGGGCGAGCACAGCCTG
GCGAGCTGATCCTCAAGCGCTGTGTTCCCATCCATCAGCAGCACAACTATGCTGCACCCTCACCCTACGTGGAGAGCGAGGACGCGCCCCCGCA
GAAAAAGATCAAGAGCGAGGCTTCTCCACGCCCCCTCAAAAGTGTTGTTCCAGCAAAAGCGAAGAGCCTGAGCCCCCGAAACTCAGACTCGGAG
GACAGCGAGCGCCGCCGCAACCACAACATCCTGGAGCGTGAGCGCCGGAACGACCTGCGCTCCAGCTTCCTGACGCTCAGGGACCATGTGCCTG
AGCTGGTGAAGAACGAGAAGGCCGCCAAGGTGGTCATCTTGAAAAAGGCCACCGAGTACGTGCACGCCCTACAGGCCAACGAGCACCAGCTCCT
GCTGGAAAAGGAGAAACTGCAGGCGAGGCAGCAGCAGTTGCTAAAGAAGATCGAACACGCTCGGACTTGCTAA
HUMAN SEQUENCE - GENOMIC
TACAGATATCTTTCCCTTATGGAATAAAAGCAGGGATAGGGCTCATTATTCCGATATTATGAACCAGGAGAAGTTAATGGACCGATCTAAAATT
CCTCAAGAAGTAAGACACAATTCTAAGCTTGAAGAACTCCAGTCCTCTCCCCGGGTGACCCTGTGTTCTTACTAGGACTACCTTATACTACCTT
GACTCTGCAGTACCTTCTTTCAAGCCTAAAGTTACTTGATTACGGACAAGCCATGAAGAGTAAAGGGGTGTGGCCCAGAGAGATTTCCAGTTGA
AGAAAGAATGATTCAACAGTCCCATTAAAACAAAGAGACATTTTCTGCAGGCTCCCCCAGGACACACATGCTATGGTCTATTGCTGCGGGGTAC
ATGGAGCCCCTGCAGGAGGGCTGGGACTAGAAGGAAGGGAGGGAACGTTTACTGGACATCCATTATAAGCCAGATGGCTACTTTGTGTAATCCT
CACATGATACTAAAAGAAAGATGAAAAAATGGCCGGGTGTGGTGGCTCATGCCTGTCATCTCAGCACTTTGGGAGGCCGAGGCGGATGGATCAC
TTGAGGCCAGGAGTTTGAGACCAGCCAAGCCAACATGGTGAAGCCCCTTTTCTACTAAAAATACAAAAAAAAAAATTAGCTGGGCACGGTGGCT
CGAGCCTGTAATCCCAGCTACTTACGATGCTAAGGCACAAGAATCGCTTGAACCCGGTAGGCGGGGGTTGCAGTGAGCCGAGATGGTGCCACTG
CACTCCAGCCTGGGCAACAGAGTGAGATTCTCTCAAAAAAAAGAAAAAAAAGAAAGAAAGCAAGAAAGATGGAGAAATAAAGCCTCAGAAAAGT
TGGACTATCCAACATAGCAGAACTCGCTGTCATGTCTGGTGTCAGAGCTGTTCTTTCCAGCATTCGCTGCACCAGATGCTGTAGAAGATGGAAA
GGCAACATGGGAAAGGGTGCTGCACTCAGGAACTGCTCATCCTGTTAGGGGAGTCGCACACACACACAGGAATCTGATAAAGAACCCCCATGGC
CGGGCACGGTGACTCACACCTGTAATCCCAGCTCTTTGAGAGGCCGAGGCGTGAGGTCGCCTGAGGTCAGGAGTTCGAGACAAGCCTGTCCAAC
ATGGAGAAAGCTAAAAATACAAAAATTAGCCGGGCGTGGTGGCACGCACCTGTATTCCCAGCTACTCAGGAGGCTGAGGCACGAGAATCGCTTG
AACCTGGGAGGAGGAGGTTGCAGTGAGCAGAGACTGCACCACTGCACTGCAGCCTGGGAGACAAGAGTTAAAAATAATAATAAAAATTAAAAAA
GAACCCCCTTGTTGGCGGCAAGCAGTATAAGATTAATGGAGATTTGGGGCAGAGAATGGGTAGGGTTAGGGGAAGGGAGGACGAGATTCCTTCC
AGGACAGTCTCCAGCTGAACTTTGAGGAAAGAATTTGAATAAGCAGAAATGAAAGGAAAAGCCTTTCTAGGCCAGAAGTAGCAGCTTCTGGAAA
TGAGCAGATAGAATCACTGATATTTATCAGTGATTGTTATCTGAGTCCAAGGAAACTGAAAGGGCCAGCAGGCAGATTCTAGCAATTCCACTCA
GATATTGAGGTAGCTCGGGCCGTCCATGGGGCTACAGGAATGTATCCAGCATTATAGGAAAATGAAATTAGCAAGAATCAGTGGGTGAGATCAG
CCATCTACTAAGTAATCACCTGAGGAGAAGATCTGAAAACTTCAGCACAGCGCGAGGTGATCTGGACACATCCAAAGCACGTCATTGTGTCTGG
AAAGTAATCCCAGAAAAAGGAGACCCCTGTCCTTCGTAGAGAGCCTGCCTGTTTCAAGGGGCAGCGCCAAATGGACTCAAAGGCCAGATCTGGC
ATCAGAATTCCTTGTAGCCCTTACAGATTCCTACTAAGATGAGGATCCCCTAATTGTGAGCCATTCCAGTGCACTGACAAATTCAAGTTTGAGG
CCCACAAGGGGAAACCTATTTCTCCCCAAACCCCATACCTGTGGAGCCTGGCCCTGAGCAGCTGCCTCCAGGGACATCTTTCCCATTTTGACTT
TTAGCATAAGGAAACTAAGGGAAATGAGAATTCCAACTCCACAGCAGCAGTATCACCGTCCATTCCCCGTAGCTCTTCATGGTTTGCAGTGCAT
TTTGACTCACTCCATCTTAGCCCTCTCTCAAGTCTACGTAATGCATGAAGCTGGAGGGAAGTCTTCACAGTGGAAGGGGTCTTTGAGCTGCCTC
CAAGGATGCAAAACTAGTCTCTGAGGGCAGTCCAGGGTCTGCTGGACACTTCGATTTTCTAAATCAGTAATCAAATTACTGGCATTAAATGAAG
ATAATTTTTGTTTTAAACCTAATTATGCTATTACAAATAAAACTTCCCACCTCCAAAACTCTCTCAGTGGTGGATCTCAAACTCTCCTCCAGCC
TCAGATGTCAAAAAACTATGCATTTTGCCTTGGGCACATCCAACATCATATACGTTGAATGGCTTTGATGGGCATGATTCTAATCTTAATTAAA
GCAGTGCCCGTTTTGGTCCATCTTGGTCTCTTTGAACCCACTCTCAGCTGACTCAGTTCAATAGCAGTTTGGTGGGAAAAATGCCAACTGGCCT
GGGAAACAAAGGAGAAATTTGGGGGTGACTTTAGGCTTCACAAAAGGGTCCCTTACATCACTTGAAATGATTAACTTTACTGAAAAGTAACAAA
ATTATTTTTGAAAAATAACAAAAACAAAGAAATTAAATTTGTTCATAACCCCATTGCTTTAAAAAAAAAAAAAAAGTTATAGGCCAGGCACAGT
GGCTCACGCCTGTAATCCTAGCACTTTGGGAGGCGGAGGTGGGTGGATCACTTGAGGTCAGGGGCTCGAAACCAGCCTGGCCAACACGGTGAAA
CCCTGTCTCTACTAAAAATACAAAAGTTAGCCTGGTGTGGTGGCATGTGCCTGTAATTCCAGCTACCAGGGAGGCTGAGGCAGGAGAATCACTT
GAACCAGGGAGGTGGAGGCTGCAGTGAGCCGAGACCGTGCCATTGCACTCCAGCCCGGGCAATAAGGCGAGACCCCATCTCAAAAAAAAAAAAA
AAAATTAATTAGGCATAGTGGCGGGTGCCTATAATCTCAGCTACTGGGGAGGCTGAGGCACAAGAATCACTTGAACCCGGGAGGCAGAGGTTGC
AGTGAGCCGGGATCACGCCACTGCACTCCAGCCTGGATGACAGAGCGAAACTGTCTCAAAAAAAATAAAATAAATAAATAAAAGTTATATCAAG
AAGAAAGTTTCTCCCTCCACCTGACTGATTGTCTTCTTCAAAGTAAACCCCTGTTAGCAACTTGGTGCACATTCTTCTCAATTTGTTACTGGGG
GGTTGATGCAGATATATCTAAGTACATATATATCCAAAATGCCAGACAGATTGACCTATACCTTGCTTATTTTCCTTCATGTTCAGGAACATTT
CCCGTCTGTAACTATAGACCTAACACATTATTTTTAGTGGGTGTATGTAATTCCACTGCATGGATGTATTACATACCATTCTCCTGTTTAATTT
GTTTTAGTTATGGATATTTAGATTGCTTATAGTATTTTTGTTATTGAGAATGATGCAGCCACACACATTCTTGTTTATATGCTTTTATGTGTTT
GTCTTATTATTTCTCGTAGGATGGATCATGAAGAGTGGAATTGCTTTTCCTCTTGGGGCAGGCACACCAACAGTGTATGTGAGGCCCCTGCTCC
CACTCCCCAGGCTGGCCAGTACTTTGGGCCAATCTGCTAGACCCAAAAATGTTAATCTCACTGAGATTTAATTTGCATTCCCCTGACTACCGGT
GAAGTTGAGCATCTTTTCATATGTTTATTGACAATTTGCATTTCTTATTCAGTAAATTACCTGCTCGTATCCTAGAGTCATATTTCTGTCTGCC
CCTTTTTTTCTATTCAATTTGTGGGAGCTCTTTGTATACCTGGGATCTAATCTTTGACTCATATATATGGCAAGTATTTTCTATTACTGTTGTC
AACTTTTTATCATAGTAAAATTCTTACATCCAGCACCCCCACCCCTCCCCTGCCCTTTTTTTTTTTTTTTTGCATTTGGGTTAGGCAGAGAGCT
AGCTTTACTGTTTTCCAAATGAAGTCAATCGATCCCAAGGATTTTTAAATTATTTAATCAGTAAAAATGTACACATTTTGAGGGAGGTGTGAAG
ATCCTAGGAGAAACTGTGCTCTTGTCCCTGAAGACAGACTCAGTAGATCTTTTTTTTTTTTCTTTTGAGACAAGGTCTCACTCTGTCATCCAGG
CTGGAGTGCAGTGGTGTGATGATGGCACACTGCAGCCTTGATCTCCCTGGGCTCAGGTGATCTTCCTGCCTCAGCCTCCCAAGTAGCTGGGACC
ACAGGCACGCACCCACCACGCCCAGCTAATTTTTATATTTTTTGTAGAGACAGGGTTTCACCATGTTGCCCAGGCCGGTCTCAAAATCCTGGGC
TCAAGCCATCTGCCCTCCTCAGACTCTTGAAGTGCCAGGATTACAGGCGTGAGCCACCATGCCTGGCCAACCCAGCAGATCTTTAATTCACAAT
CTGGAAAAACCATCTAGAGGCTGTTGCTCTCCTAGCCAGTCATGATTTGGGCCCTTCTCAACATGTGTATGTGTGTGTTGTTTCTTTGTGTGTG
TGTGTCTTAGTAAAGAGGAGATTTGTAAACTAAAATATTCTCCTCTCTCAAAAATCACCTTAATTATAAACCAAATGCTGTGTATAACTCTATC
CTAGCAGTTGGTGATTAGATTGAGGCATCCATTTCAGATGTGGGTCTCCTTCCCTGGTCCACAGTGAGCTCCTTGCGGCTGTACCTTAACTTAC
CTCTGTTCCAAACCCACCCAGGGGCTCAGACACAGTAATAGATGGGTACTTGTCTCAGTTCATTTTGTGTTGCTATAACCTAATAGCACAGACT
GGCTAACTTATAAAGAAAAGAAATGTATTTCTCATGTTTCTGGAGGCTGAGAAGTCCAATATCAAGGTGCCAGCAATTGCTGAGGGCCTTCATG
GTGTACCATCCCCTGGCAGGAAAAAGGAAGAGAGGGCAAGAGATCAAACTCCTAGCCTCAAGCCCTTTTATAATTAGCATTAATCCATTCACAA
GAGTGGAGCCCTCATGACCTAAACACCTCACATTAGGTCCTGAGACAGGAATAATATAAGGTGGCTGCAGAATAATAGAAGATTTTGGGCAGCA
ATTTCACATGACTAGCAAAAGGAAACTGTTGAAATGGCTGCAGAGGCCATGGGCTAAGACCGTGAAAAACAGAGTGTAGACCAAGCTGGCTAAG
ACCCACTGAACCCAGCATGGCACTGGATTTGACCTAGGTTTCTCCTAGGACCTCATTATACGCTCATTAACATACTAAATCACACACCCCACTA
GCATCATGACACTTCCGAGAACACCCATATTTGGTTTAAAAATAGGTGGTGTCACAGTTCTGAGAAATCTCCTCCTTTTTCTAGGAATGATCAT
GAATATTCCGCCTCTTGGTTAAAGAAACCCGTAAAGGTAGCAACCCCAAACCCCCTTTCGTGTGAGTCTCTCGAGTACACCCATATTCCCTTTT
CTTGAGTGTGTACTTTTCCCTTTGCAATAAATCGTACTTTCACTATTTTCCAACTGGTCTTTGAATTCATTCTCTTGATGGTGTCAAGAGCCTG
GACACTGGCTGGGGTCTAGGTCCCACTGGCATTTGGGGACCTCCCCTCCAACACTGTTGCACTGGGGGTTAAACTTCCAACGTGTTTTTTTTTG
GAGGACACATTCAAACCATGGCAGTACTGAACAAAACCTTGGCTTTGAAACCATATGAAGATTTGTTTTGTGTGTGTTCACATAGTTGAAGTAT
TTCAACATTTTTAAGAGGGATTTTCAGGCTTCTCTATAAGCAGACTAAGAATCAGCATTAGCATGTTTTCTCCATGTACAAAGTCTTTTATCTT
TCTACTTACAAATGAAGGTAACCAATAAATATTTGTTGAATTTTATCATTCTGTTAACCTTCAGATTCAGACTTGTCAGTAATTACTGGCTTGC
ATTAAAAGCAGGAGATTAGAGATTGAGGTCCAGGAAGTATAAATGATTTGCTCAAAGGCATGTAGAGTAACCAGGTCTCCTGTCCCTCTGTCCC
CCTTTTCTGCTACACCTTGTCCCTCGACAAAGGGTGAATGACCTAGTGATGGTCATTCATCTGAGGAGAAAGGACAAAGGTAGGACTTTGAATC
GGAGCAACTGAACCCATCCTGGCCTATTTTCACAGTCTTGCCACAGAAGATCTGGCCACCTTAGAAGGACTCATTCAGTGATCTTGGTCTTGAA
TAATACCATGAGTACTTACTATATACAGGTACACTGCTGTGCCGTAAGGATTCCCTCTGGAGCAAAACTGACCCAGGTCACAGGTTAGTGAGGG
AGACTGAGCCTGTCTTCCACAGTGTCACGGAAGTGAGCACTAAACAAATACTCCCAGGCATGGATCATTACAAATGGAGGTGAGTGCTGGACAG
GAAAGGACAGGATGCTAGGCGAGAATTCAGCAGGAGGGCTTGGTGGCGATTAGGGGGCTAGGGAAAAGCAGAAGTGGGGCTGTAAAAGGCTTTT
GCAAAGATGAAGGCTTTTACATTTGTGAGGGAAATAACAATAATAAAGGTGCTTGGAAGGGCTGCTCATCCGCATTCATGGGCCTCTTCCTAGA
ACTCTGGGAATCTTTCTCCCTAGAGCTCTGTAGGCGTCCAGGGTCAGGGCTGGAAAGGCCTTCACAAATGCTAATCTGTGAGCATTTTATGGGT
TGCCCCCTGACCCTCAGCATCCTCCTCCTTAACTAGATCCAATGGTGAGGTGAAGAGGTGGGGGCGATGGAGAATGGCATTTCCAGTTTGGAGA
AAAAAGTAGGGAGAGGCGGGGAAGCCTTCCCACCCCAATCCCAGGGCCAGGTTCCCCTCTCTCCAAACAAGCCTAAGGAATGTTTTTCAAGGCA
AAATCCCACCTCACTGTGTATTTGGTTGTGAAGGGGGCTGTCCAGGAGATCAGGGTTGTTTTTGGAGAGTTTGGAGGGCAGGGGCAATAATGTC
TGACCCCCTGCAAACAAATACACCTGTAAGAAAAGCTAGACCAGAGTGAAGTAAAGCCAGGATTTGATGGTGGCGGGAAGGGAAGGGGCCAATG
CTGCTGCTGGACAGAGGGGTGTTATTTTCCCCAGAAGAATAGCATGTCGTGGTCATCATCATAATAATAGCTGACACAACTATTATGGGCTGGG
GACTTATAAGAGCTTTCATAACTCTCAGGTAGGAGGCACTACGAACTCCATTTCACAATTGAGGAAACTGAGGCACAAAGAAACGAAGTAGTGA
CAGCAGCTTTCCTTTTTCCCTTCAACAGTTTGGAATCAAGCTGTTTGAGCCGAGGCTGGGTCTCAGGAGGTGTGGACAGCACTTCCATCTCGGT
AAATCAGATTCGCCTGCCCATAATTGGGGAGAATGGTGGCTTTGAAAAGGTTAACTTGGGAGCCCTGGGGACGGCTGCAGGGAACGTACGCCCA
TTCCCCCTAGGAAGCAGAGCCAGGCCGCCTCCTCCTCCGCAGTGGTGAGGACGCGTGGACAGTCCCGCGGGGGCGGCGGGAGACCTGCGGAGTG
GCCGCACTCCAGGTCCGCGCCAAGGCCGGGCAGCTCCGCTTTCTGCTCAGTCTCCGCGAGGTGTCGCCTTCGGCCGAAGAAACCACCGCGGCGC
CACCCTCGTAGCTCGCACTTATTTATTTATTTATTTTCAAACAAGGGGGGCGCCCCTCTTCTTTCAATTTGAAACTGGAAACATCCAGAGGTCT
TGTTCCTAAGGGGGCGCGTCCTCTCCCTGCTATTTTGCACCTTCGGACTAGCCTTCTTTCGTAATTACACAGGAGCAACCTCCCTGCAAGGCCT
TGCTCAACGTTGGCCTCGCGCTCAGCTGCACAACACGCAGTCAAAGCGGGGGCTGGGTTAGAAGCATCGGTCTCCCCTCCCCAACACACACCCC
CGGAGCCCTCCGTAATTTTTTTTTCTTTTAATGACAAGCAATTGCCAGGCTCGCAGGGTGGGTGCTGCATTGCACCGCTCCGCGCGCAGCTGGT
TCTCAGAGTGCAGCCGGTGCAAGCCCGGGGGTCCAAAAGGGCGGGAGGAGCACACCCTGGGCTTCCCAGCTTTGCAGCCTTCTCTCTGCAAAGA
AAAGCAAGTGGCTTTTGGCGCGAAAGCCTTGGCGCCTCCCCTGATTTTTATGGAAATCAGGAGGGCGGGGTAAAGCCGCTTTCCTCTCCTTTCT
CCCTCCCCCTTGTCTGCGCCACAGCCCCCTTCTCTCCCCGCCCCCCGGGTGTGTCAGATTTTTCAGTTAATAATATCCCCCGAGCTTCAAAGCG
CAGGCTGTGACAGTCATCTGTCTGGACGCGCTGGGTGGATGCGGGGGGCTCCTGGGAACTGTGTTGGAGCCGAGCAAGCGCTAGCCAGGCGCAA
GCGCGCACAGACTGTAGCCATCCGAGGACACCCCCGCCCCCCCGGCCCACCCGGAGACACCCGCGCAGAATCGCCTCCGGATCCCCTGCAGTCG
GCGGGAGGTAAGGAGCAGGGCTTGCAAACCGCCCGGCGCCCAGGGAAGCGACGAGCGCCGGGGCAAGGCAAGCCCTGGACGGGATTGCGACGTG
CGCACCGGGCGCCCTAATATGCCCGGGGGACTGTTTCTGCTTCCGAAACAAAACCATCTCTGGGTTTTCCCAGAAAAGCCAGTTCCAGCCCCGA
AGGCATCCTGGCTAGAGGAGACCCGCCCTAATCCTTTTGCAGCCCTTACCGGGGGGAGTAATGGCTTCTGCGAAAAGAAATTCCCTCGGCTCTA
GAAGATCTGTCTGTGTTTGAGCTGTCGGAGAGCCGGTGCGTCCCCACCCCAGGCTGGGGTTCTTCTCCAAAGGGTGCCCCTGGAGGAAGAAGAG
GGGGGGATTAGGCAGGGCGAGGCCGCCGCGGTCGCAATCTGGGTCACGGCTGCTCCAGCTTGGAGGAGAGGCGGCTCTCCCGGCGACCCTCCTC
GCGCGGGCGCCCCTGCCATTCCCGGGAACAGGGGCTCAGCCTCTCCCTCCCTGGAAGAGGACGTTGTCGTGGGTTTGGAAGAGCAGGGGTGGGC
TTAGAGAGCTTCCAATTAAGCTATTGGCAGGAGTATCCCTGCAGCGGGTGAATGCCGAGGGGCGTTTGCTCAAATTTGGGGAGGGGAAGGATTT
GTGGATATGGGTGTCTGTTGTTGGTCTCTGTCTAGAGAAAGGCTTTTTTTTATTTGCAAAGTTTTCTAAATCCCCTGCTATCATTTGCACTCCT
GAGGTTGCATTTTTACAAAGGGGGTAGAAGGTACTCCAAATACCATTCCCGGTAGCTGGGTCGGAGAGCCTGGGGCTTCCCCTGAGCAGCCGGC
CCCACACCGCTGCGAGTGCGGTTGTCTGCGTGCTCGTGAGAGCTAGAATTCTGCAGCCAGGAACAGCCCCCTCCCCCAGGCAGTGCCTTGTGTG
AATGAAATGGCAGTTTCCAAAGTTGCGGAGCCTCGCCACCACCCCCTGCATCTGCATGCCCCCTCCCACCCCCTGTCGTAGACAGCTTGTACAC
AAAAGGAGGGCGGGAGGGAGGGAGCGAGAGGCACAACTTCCTCCACCTTCGGGAGCAGTGGGCAGAGTGGGGGGCTTGGAGGGAAGATTGGGGA
ACCTGGTTAGAGGGGGCGCCCATTGCCTATCCCCTCGGTCTGCCCCGTTTGCCCACCCTCTCCGGTGTGTCTGTCGGTTGCAGTGTTGGAGGTC
GGCGCCGGCCCCCGCCTTCCGCGCCCCCCACGGGAAGGAAGCACCCCCGGTATTAAAACGAACGGGGCGGAAAGAAGCCCTCAGTCGCCGGCCG
GGAGGCGAGCCGATGCCGAGCTGCTCCACGTCCACCATGCCGGGCATGATCTGCAAGAACCCAGACCTCGAGTTTGACTCGCTACAGCCCTGCT
TCTACCCGGACGAAGATGACTTCTACTTCGGCGGCCCCGACTCGACCCCCCCGGGGGAGGACATCTGGAAGAAGTTTGAGCTGCTGCCCACGCC
CCCGCTGTCGCCCAGCCGTGGCTTCGCGGAGCACAGCTCCGAGCCCCCGAGCTGGGTCACGGAGATGCTGCTTGAGAACGAGCTGTGGGGCAGC
CCGGCCGAGGAGGACGCGTTCGGCCTGGGGGGACTGGGTGGCCTCACCCCCAACCCGGTCATCCTCCAGGACTGCATGTGGAGCGGCTTCTCCG
CCCGCGAGAAGCTGGAGCGCGCCGTGAGCGAGAAGCTGCAGCACGGCCGCGGGCCGCCAACCGCCGGTTCCACCGCCCAGTCCCCGGGAGCCGG
CGCCGCCAGCCCTGCGGGTCGCGGGCACGGCGGGGCTGCGGGAGCCGGCCGCGCCGGGGCCGCCCTGCCCGCCGAGCTCGCCCACCCGGCCGCC
GAGTGCGTGGATCCCGCCGTGGTCTTCCCCTTTCCCGTGAACAAGCGCGAGCCAGCGCCCGTGCCCGCAGCCCCGGCCAGTGCCCCGGCGGCGG
GCCCTGCGGTCGCCTCGGGGGCGGGTATTGCCGCCCCAGCCGGGGCCCCGGGGGTCGCCCCTCCGCGCCCAGGCGGCCGCCAGACCAGCGGCGG
CGACCACAAGGCCCTCAGTACCTCCGGAGAGGACACCCTGAGCGATTCAGGTAAAGACCGAACTCGGGTCCGGCTGCCTCCCTGGGGCACTGGA
CCCCGGGTCGCGTCCCCTTTGTTAGTGCTCGTATGTCTTGGCCTGGGGAGCATTTTGGAGGCAGTGCTAGGGGCAGAGAGGTCCTGTTTCCCCC
AAGTCTCTCCTCGGGGTAAAGAGAAGGGGCTGAGAGAATGCCGTTGCAAAAGGGGTGCTCTCCAATTCTCGCCTTCACTAAAGTTCCTTCCACC
CTCTCCTGGGGAGCCCTCCTCTAGGCCATCACGGGCCCTCACCCGGTCCCCCACCTCTCTTTTGCAGCGCAGTCTGAGGAATAAAATTGGAGAA
AGTTGGTGGCTAAACCGGGTGGGGGTTTAGGGGGTTGCTGGGTGCACTGCCTGGACAGAAACCTGTTAGCGCAGGGGTGAAAGGGACTCTCTGG
CCCAGGTCAGGGGAGGGAAAGACATCCCGAGAAGATTCAAGGGCTGTGCAAAGCCCTGTTTAAGGCGCAGGAACTTATAGGAGGGTTGCACAGA
TGGCTAGAGCCGATTTTCTATTCTTTTTCTTTTTCTTTTTTTTTTTTTTTCAAATGTCGGTACCTTTCCCTTCCCCCATCCTCGGTGGGTGGTG
GGCTATTTGCTCCTGGTGCGTGGCCAGCAGGCGGCGATATGCGAGGCCAGCAGGCGGGCCCGGGATCTGAAAGGCTGGGGGTGGTGGGGGCACC
CTCCCTCCCTCCATTCAGCAGCTGGCTGCAAGTGCAACAGCAGTTGTGTACATTCTCAGGGGGCCTCCTCTTTCCAGTGTGCAGTGGAAACTGG
CTGTAGTTTTGTCTTCCAGCCTGAATTCCAGGCCTAATTTGAGATGTGAGTTGTATCTGTAACCCAGTGCCCTTGAAGGTGAGGGCAGGCACTC
AGCAGCCTCTCCAGGAAGGCTCACATCCTGGGAGGACTCACTGATTAGTTCTATTGTGTTCATTTGTCTGTGTCTTAAGCTGAAGGGAAGAGTT
AAAACCAAGCCTTTCCCTGGGGGTCTGGATGAACAGAACTCAACCCAAAGAGTGGCATTGCCTTGTCCTTGGAGCAGGGAGCTGGGACCCCCCT
TGGACTTTGAAAACCAGTGTTTTCAGAATGCAGGTGGATAACAAGCCTAAATTTACTTCTGGGCTGAGGAGAGATCTTTGAGGCTCCTGGAAGG
AAACTTGGTGATAAGCCTCCAGTTTGAAACGGCTCTGTCCCTTTAATGTCTGTGCCTTGACAGCTTTTGGTGAGGAAGCACTTCCTTCCAACAG
CTGTCTTCTTGGCAGAAAACCAAAACATTGGCTTAAAGGGACCCACAGACTGGAACAGCCTCACATTTCGGCTTTAGAACAAATCCCACAATTG
TTCAGCTTTCCGGTCCCCTTCAGATCAAGCAGAAGATATGTTTTGATTTTCATGCTTGTATTTTAAACAATAATTTTCTACCCCAGCGTGGTAG
TCAATGAGGAGAGAGGGGAAGAATGCGCACATGATGCTACACGTTTCTGTTGTTGCTGTTATTATTGGTGGCTTTGAGGAGAGCTGCTCCCATT
TGGGGGTTTATACCAACTGTGGATTATGGCTTTGTCATTAAGATTTGATCTTTGTTAAATGAAAAACTGTTTATTGTATAAAACTCAGGTTTGT
GGACGAAAAGTTGTTTTTTTTCTTCAGTTAATTAAATTGTTCCTCAAGTTTGTTTAAGGACTTAAAATCAAACACAACCATGTGTAAACTGCTA
AATGAGGCTCCTAAAATGAGAGGCCTCAACTCTTTAAGTGTGGAGCTAGAAATGTAAATAAGTCCACAGGGCAGACTGGTGATTATGATAAAAG
CTACCATTTACTGAGCATCTGTCTACTAGGCTCAGCTCTATGCTAAGTCTACATGTTATCTGTCAAAGTGGTATCATCCCCATTTAATAGCTGA
GGAAACAGAGGCTTAGAAAGGCTGGGTAACTTGACCAGGGTCATGCAACTAGTCTGCGGTGGAGCCAGGATTCTGTCTGACCCTAAAGGCCAAG
TTCTTTATATTTATTTCTACCACCTGCTAAAGTCTTGAATGGAGGCTGAAAGCACAGTTGGGGTATGGGGAAGAAAAATATATATACATACATA
TATGTATATGTATGTATGTATGTATGGGGGGTTGTTTTGTTTTTGTTTTTGATAAGGAGTTTTGCTCTTGTTGCCCAGGCTGGAGTGCAGTGGT
ATGATCTGGGCTCACTGCAACCTCCGCCTCCCGGGTTCAAGTCATTCTCCTGCCTCAGCCTCCCGAGTAGCTGGGATTACCGGAGCATGCCACC
ACACCCAGCAAAGTTTTGTATTTTTAGTAGAGACAGGGTTTCACCATGTTGGCCAGGCTGATCTTGAACTCCTCATCTCAGGTGATCTGCCCGC
CTCCGCTTCCCAAAGTGCTGGGATTACAGGTGTGAGTCACCGCGTCCGGCCTACAGATATATTTAATTTAAAGAGATCTAAAACAAATACAAAA
CTGTCCACATCTATGTTGATGGACCCATAAAAATAGCAGTCTGCCAGGGTCTGCCGGAAGAGACAGATAAGCATACATATTAACATGGATATAT
ATGTGAATTTCATTCAAATGGTTCTCACATGAGAGTAACTAGCATCTTTCTCTCAGATGATGAAGATGATGAAGAGGAAGATGAAGAGGAAGAA
ATCGACGTGGTCACTGTGGAGAAGCGGCGTTCCTCCTCCAACACCAAGGCTGTCACCACATTCACCATCACTGTGCGTCCCAAGAACGCAGCCC
TGGGTCCCGGGAGGGCTCAGTCCAGCGAGCTGATCCTCAAACGATGCCTTCCCATCCACCAGCAGCACAACTATGCCGCCCCCTCTCCCTACGT
GGAGAGTGAGGATGCACCCCCACAGAAGAAGATAAAGAGCGAGGCGTCCCCACGTCCGCTCAAGAGTGTCATCCCCCCAAAGGCTAAGAGCTTG
AGCCCCCGAAACTCTGACTCGGAGGACAGTGAGCGTCGCAGAAACCACAACATCCTGGAGCGCCAGCGCCGCAACGACCTTCGGTCCAGCTTTC
TCACGCTCAGGGACCACGTGCCGGAGTTGGTAAAGAATGAGAAGGCCGCCAAGGTGGTCATTTTGAAAAAGGCCACTGAGTATGTCCACTCCCT
CCAGGCCGAGGAGCACCAGCTTTTGCTGGAAAAGGAAAAATTGCAGGCAAGACAGCAGCAGTTGCTAAAGAAAATTGAACACGCTCGGACTTGC
TAGACGCTTCTCAAAACTGGACAGTCACTGCCACTTTGCACATTTTGATTTTTTTTTTAAACAAACATTGTGTTGACATTAAGAATGTTGGTTT
ACTTTCAAATCGGTCCCCTGTCGAGTTCGGCTCTGGGTGGGCAGTAGGACCACCAGTGTGGGGTTCTGCTGGGACCTTGGAGAGCCTGCATCCC
AGGATGCTGGGTGGCCCTGCAGCCTCCTCCACCTCACCTCCATGACAGCGCTAAACGTTGGTGACGGTTGGGAGCCTCTGGGGCTGTTGAAGTC
ACCTTGTGTGTTCCAAGTTTCCAAACAACAGAAAGTCATTCCTTCTTTTTAAAATGGTGCTTAAGTTCCAGCAGATGCCACATAAGGGGTTTGC
CATTTGATACCCCTGGGGAACATTTCTGTAAATACCATTGACACATCCGCCTTTTGTATACATCCTGGGTAATGAGAGGTGGCTTTTGCGGCCA
GTATTAGACTGGAAGTTCATACCTAAGTACTGTAATAATACCTCAATGTTTGAGGAGCATGTTTTGTATACAAATATATTGTTAATCTCTGTTA
TGTACTGTACTAATTCTTACACTGCCTGTATACTTTAGTATGACGCTGATACATAACTAAATTTGATACTTATATTTTCGTATGAAAATGAGTT
GTGAAAGTTTTGAGTAGATATTACTTTATCACTTTTTGAACTAAGAAACTTTTGTAAAGAAATTTACTATATATATATGCCTTTTTCCTAGCCT
GTTTCTTCCTGTTAATGTATTTGTTCATGTTTGGTGCATAGAACTGGGTAAATGCAAAGTTCTGTGTTTAATTTCTTCAAAATGTATATATTTA
GTGCTGCATCTTATAGCACTTTGAAATACCTCATGTTTATGAAAATAAATAGCTTAAAATTAAATGATGCAACTCAACCTTTTCCTTAATGGCA
TTACACTCTGTCCCTTAAGGAGCAACCATAAATAATCCATAACCTATAGAGGGAATTTGGTTTCCGTAAATAGCCCTTTTTGCACCTGTACAAT
CCTGGTTGGGGGGCATGAGTCATTGTCCCCACTTAAGGTGGAGGGAACTGAGTTTGGGGAAATTAAGGCAGTTCTCCAAGATTATGCAGAATAG
AGATGTTATTAGCGACTATTGTGTGCATTGTAGCAATGGCATTTGATAATTTACAGAGCACTTCTGTATACTGTGGCTCCTTAGTGGAATTAAG
CTGAGACCTCAGATCAGTCCCTTTAAAAGAAAAGTAAAAATAGCCACAGGGTTGTTTAACTCGCTTGTATTGGGCTTTGGTAGTATTCGTCCCA
TTGGCAGACAGTCTTCTATTTTAAGGTAGAGCATAGTTTGTCTCCAAGAATTCATTGTTAAAAACATTCACTAGGATCTGTGGAGGTTCTAGGC
TAAGCAGAGTAAGCAGAATAAACAGATGGAAATGGGTTCCTCCCTTCAATGAACTCAACCTGATTCACCAAAGTGTAATTCACGAGTGACAGTT
ACGTAGTCCAGAGCAAGTACCAGGTGCAGGAGAGACCCTGGGGTCACAATGGAGACCCCATATGGGTGGCATGGCTCTCTACTTGCCATTCCTA
TTACACAGCAGTGTCTTGGCTACTTGGGCTGCCCTTTGGAAATGGGATCAATCTTGCAGGCTCCAGAAGACAGAGCCAAGACTTGTGTGTTAAG
GAGATAGTTTACCTGTTGGGACATAAGAGCCTTGCTTGTAAGCAGCTTCTCAGAACTAAAAGTGTACAAATAACCTATTTCTCTTTTGAGTGTC
AGGTTGTACTCGGTTATTTCTCTAGGGTTTTGTTCATCTCAGATTTTTAACTGTGAGCCGAAAAAAAAGAAAAAGGCCTTTTGCTTCACTGAGA
AGAAAGCAGGTCCAGAGATGAGACAGCTTTGCTTGCCTGGACACACAGTTTGTCAGGGGCAGAGATGGGAGACACCAAGCCATGTGTCCTTGTC
TGCTTTGGTCATTTTTATTTGTTTCTTTTGAGATGGAGTCTCACTCTGGTGCCCAGGCTAGAGTGCAGTGGTGCAATCTCGGCTCACTGCAACT
TCCGCCTCCCGGGTTCAAGCGATTCTCCTGCCTCAGCCTCCTGAGTAGCTGGGACTACAGGTGTGTGCCACCATGCCCGGCTAATTTTTTGCAG
TTTTAGTAGAGACCAAGTTTCACCATGTTAGCCAGGATGGTCTCGATCTCCTGACCTTGTGATCCACCTACCTCGACCTCCCAAAGTTCTGGAA
TTGCAGGCGTGAACCACCATGCCCAGCCGCTTTGGTCATTTTATAAAGGCAAGTAGGAGTTTAGAACTCAGTCCTCCTTATCCTGCTTATCGCA
CACCCTTGAGATACGAGGTTGGTGATACTACCATGGACATCTTTCTGATGGTCTCGCCATGTAGAAAATAAGATAAAATGCCCTTTAATTCTCC
TAGTCCCCATCCCCAGACTAAAAAATAATGCACCCTTTCTTTTTGAATTGTCATTCAAAAAACTTCCGTTCGTTTGCATTTTACAGGCCCCAAA
TCACTCACGGCCTCTGCCCCTGTGTGTGTGCAAGAGCCAAAAGAAAGTTAAGCAACCTCACATTAATTGTGTACCTAAGATTCTTGAAGCCCAG
CTCAGTCATCCCAGTTTCTCTATTTGTGATTACCATAAGGAACCTACCTGAGTGTGTCCAATGAAATGGAATGCTTAACCGTGAAGAAAGAAAA
CTGTTCATATGAATGAGGCCACTAAACTACAATTCCTACTATGCTCTCTTTTTGCTCGGGCTGCTGGGAAGCTCAGAGCCAAATTCCACCCCCT
CTAACATCTCCAGTGGTCATTCTCTTCTAAATGTTTACGGTTTCTTGTTTTCCATTGTTAGGTTAGTCATCTTTTTAATGTATATAATGTAGTA
TATAACATGAAACGTTTTTGCATTAGGTGGACCACAAATATGCACCTAAAGGATTAAAACTAAAGTGAAAAAATCCCATATTTGCCTTTTCTGA
CCTAGCTGAGGGAATGTCACCAAATTTAGCTCTGCAATTATCTATAAAGTAATTGCTGTAGTCACAGAAGAATTGAACTAGTTGTTTCAATAAC
ATGAGAAACCATCTCTGCATTCCAAGAGCAGTTTTGAGACTGGGAAAGAGCTCTACATCATTTAGTTCAATAAACGATAGAGCTGAGACGAAGC
ACAGAGATCATCTCATTGAAATTCATTTCTCAGAATGAGGGTGCGGGATCCCACCTGGAATTAATGGCTTAACCCAGGGAGACTAGAGCCGGTG
TCTCCTATGACCCGCCCAGTGCCCTTCCCCATAACATTCACTTTCTCATTGCCCACACTGTAGCCTCCCCACCTCCTGGCCTGCCCGTCCACCT
ACATCATGCTTCCATATTAACCCTTCTAAACCACCTACTCTTTATGCAAACTTCCAACAGTTCCCTACTATTTACAGAATCGAGTCCCGACTCT
CCAGCCCAGCACAGAGGCACTTCTACCCAGTCCCACCAGCTCCATTGCCCATGGCCCTCCATGTGCTGGACTCTGGCTGTTTCTCATCCGTGAC
TTGTTTCACCACATCCCTCCTTCTAGGTTACCCAGATTCCATTAATTGAAATCCTTCTCAATCTTGCACCCCCAGGTAGGCCTTTCCCAGCCCT
TGGCCTCTGTGCACTTATCTTCTAGCTGCCTTATGCACCTCCTGGACCCCCTAAACTGTGGGCTCCCTTAAGGCAGATAGGGCTCTCATTTGCC
TCTGATTCTGAGCATCTGCCCTGTGACTGACAGGTGACAGAGCCTTAGCAGATGCCAGCAGTATAGACGGTAACAGAGGAAGGAGCGAGGAGAG
GATTTCCATTATGGGTGGGTCTCCCACCAACTCCAGTTCACAAGCTCTCAGGCTAATTCAGATGGGAAAAGACATCCCTTAGACATCAATGAGT
GTCTGGTACTGACTATGCTGGCAGTTCTGAAGAAGTGAAGATAAAATGCTCATAGAGGACTGTCTACAAGGAGCCCACAGTCTGCAAGAGAGTT
AAAGCCCTGGGGGTTGTATAGAAGGCAAGAAGAGAAACCAAGAGGCACTGGGTTGAGGGGCTGATGAATGAAAGCATATGAGGGGCCTAGTGGC
TGAGTTTGGGGCTATCTGAAGGAGGAAGCTGGTCCTCTGCCTGGGTTCACTGAGTCTACGCCTGGCCTGGCTTGTGGCAGCTCAGACACTTGCC
TCAGGCCACAGATGGTCTACACTCGTAGTGAAAAAGGTTGCAATGCCTCCCACCCCTGTGTAGCACTGCAGAGCTTCCCAAGTTTTTGTATGCA
TGAGTCTCAAAAATCCTTGAGGATTTTTGACCAGTTTATCACATTCCCACATTATAAGTGAGGGAATCTGGTCTTCTGAGTTTTAGTTTCATTA
ATCCCTATGGTGGCTATGTTTTGAAATTCTGATGTGAGTTGTTTTCGAAATCAGAGGTTGTTTGATTTGGAAGAGGCATAGAAGCCAACTAGTT
TACTCTCCCATTTCAGGGGAGGCACCTACAGTCTGCTTGTGAGAATTTTATTTTTCCTTTATTTTATTTTTCTTTTTTTTGCAACAGGGTCTTG
CCGTGTTGCCTAGCCTGGCGTGGAGTGGCTAATCTCAGGCACAGTGCCACTGCTGATCAGCACAGGAGTTTTGACCTGCTCCATTTCTGACCTG
GGCCAGCCAGTTCATACTTCCTTAGGAAACCTGGTGGTCCCCTACTCCCAGGAGTTGCCATATTGATGCTAAACTTAGTGTGGACATCCAGTAG
GCATAGGGTGCTACAGCTCAGAACTTCTGGACTCAAGTGATCCTCCTGCATCAGCTTCCTGAGTAACTCGGACAACAGGTGCACACACACTGCC
GTGCCTGGAAGCGTTCTTTTTTCTTTTTTTCTTTCCCTCTTTCCCATTCTTTCTCTCAACAAATATTTATTTAACAGTTACCATGTATCAAGCA
CTGTTCTAAGTGCTTGGGAGACATCAATGAACAAAAAAGACAAAACTCTCCCAAATCTTCTCTGCTTTCTTGAAGTTTATAGTGTAACAGAGAG
TGACAGCTGTACAAATAAACATAATAAGCAAATAAATTAAACAGCATCTAATATGGTGGCAAATGCCGTAAAAAATAAGTAGAAAGGCTAGAGG
AATTCAGGGATGCCAACAGCAGAGGTAGGAGGCTCAGGGAAGGCGCATCTGGTCCTGGGAGCTGAGGGGCAGGGCCTGTGGGGAGAGTGTCCTG
CCCTGGGCACTCTTGTGCTTGCTCAGAAACCAGATCTCAGGCCTCAAGCTTCTCGGTTCAGTGCTTAACTTCCCTTATCTCTGGCTGGGCGTGG
TGGCTCACGCCTGTAATCCCAGCACTTTGGGAAGCCAAGGCAAGTGGATCGCAAGGTCAGGGGTTTGAGATCAGCCTGACCAACATGGTGAAAC
CCCATCTCTACTAAAAATACAAAAATTAGCTGGGCATGGTGGCTGGTGCCTGTAATCCCAGCTACTCAGGAGGCTGAGGCAGGAGAATTGCTTG
AACCTGGGAGACGGAGGTTGTAGTGAGCCGAGATCACACCACTGCACTCCAGCCTGGGTGATAGAGCGAGACTCCGTCTCAAAAAATAATAATA
AAAAATAAAAAATCTTCCCTTATCTCCCTCTGGACATTGGTAAATGTACCCCACCAATTTCCATTCATTGTCTCGAGGAGAAACCATTGAGCAT
AGTAGTTAAGAGTATGAATCTGGGCTGGGCATGGTGGCTCATGCCTGTAATCCCAGTACTTTGGGAGGCCAAGGCAGGTGGATCACCTGAGGTC
AGGAGTTCAAGACCAGCCTGACCAACATGATGAAACCCCATCTCTACTAAAAATACAAAAAAATTAGCCAGGTGTGGTTGCGGGCACCTGTAAT
CCCAGCTACTCGGGAGGCTAAGGCAGGAGAATTGCTTAAACCCAGGAGGCGGAGGTTGCAGTAGGCCAAGAGCGCGCCATTGCACTGCACCCTG
GGTGACAGAGCAAAACTCCGTCTCAAAACAAAGAGTATGAATCTGAAGTTAAGACTCCCTGGGTTTGGCCGGGTGCGGTGGCTCACGCCTGTAA
TCTCAGCACTTTGGGAGGCCGAGGCAGGGAGATCACCTGAGGTTGGGAGTTCAAGACCAGCCTGACCAACATGGAGAAACCCCGTCTCTACTAA
AAATACAAAATTAGCCAGGTGTGGTGGCTCATGCCTGTAATCCCAGCTACTCAGGAGGCTGAGGCAGGAGAATTGCTTGAACCCGGAAGGCAGA
AGTTGCGGTGAGCCAAGATCATGCCATTGCACGCCAGCCTGGGCAACAAGAGTAAAACTCCATCTCAAAGAAAAAAAAAAAAAGTTAAAGACTC
CCTGGGCTTGAATTTGATTCCCAAACTCACAGGCTCTGGGTTGTGCAGCTCCCCAACTGGTACAACGAGGGGATAACACTGGGATTATTAGCGG
GTTGGCATCATTATTATTAATATGTTTTAAAATGGTCTCACTCTGATTGCCCAGATTGGAGTGCAGTGGCCTGATCTCGGCTCACTGCAGCCTC
GACCTCCTGGGCTCAGGCGTTTCTCCCACATCAGCCTCCCAAGTAGCTGAGACTACAGGCGCATACTACCATACCCAACAAATTTTTTCCATTT
TTAGTGGAATGGGGTTTTACTGTGTTGCCCAGGCTGGTCTCGAACTCCTGACTTCAAGTGATCCTCCTACCTTTGCCTCCCAAAGTGCTGGGAT
GACAGACACAAGCCACTGCACCCCGCCTTCAATTGATTTATATACAGCACATAGAATAGTGTCTGGCATGTGACAGGTGTGGTTTAAGCCTGCT
CAATAATATGATTTGATGTTGATGGGGTCTTTCCTCTCTCTATTTCCTCAGTATGTTACCAGGGCAAAGAACCTTTAGAAAAAAACATTTACCG
GTAGAACAAATTCACAAGCCTGAGTTTAACAAGAACTTTGCAGACTTTTGTTCCTCAAAAGAGGCCTTACAAAACCTGAAAACGGGTACCTTGG
AAACAAATGAATGTGAGCTTTGAAGCCTATTTTAAGACGGAGCCTAGGAATTTGAGTGCCGCAGAACATCTACGCCTTCTACAATGGTTTATTA
GTTTTCCTTACTTGTAAAGGAAATCTCTTCATAGGCCACTGACCAGGGTTACCTGATCTCTTTTGATATTGGTGTATCAAGCTACAAGACATTC
ATCCTGAAACCTTTGACCAGGAGAGGTAACAGAGCCTTTTTATGGTCGAAGCACACAGACCTATTGGTACAATGCACGTTTGGAGAGCACACAC
AGCACAAACTCCTCTGATAGCTACAGACAATTTGCAATGGGATTTGCTCGGAAGAGGCAACACAAAATCAACTTTTCTCTGCATTCCAGTGGGC
CTGAGATTCTCAGCAGACCTTTGAGGTAGGGTGGGGGAAACAGGCCCACTCTTTCACAAAGCTTGACATCAGTTGAATTTTGTGTGTTCTTGTT
TTGTGGCTCAGGCTATCTTCAGCCATCTTCCTGAGAGAGGACAAAAGAGCAGTCCCTCCACTGGAAAAATGTTGTTCCAGGTTCACTGGGAGGC
AATCCTGAGTCACAGAAACACTTAGTTTGGAAGAAGCCCCTGGATTGGGTCTTAGATCAGTTATTGACTATTTTCAACCTTGAAATAGGAAAGT
AACTTCTTGTTGCTCAGTTTCCTTGTCTATAAGTAGAATGGGCTGAAGTCAGTGATCTCTGGGGCTCCCTGTCAACTTTGAGGATCCAGAGTCA
TTGGTCCTTTGCAATGGCTGCAGCTTTAAAGGTGGCTGCAAGACTGCAGTGTCTGTCAACACCCTCTGGTTGGAAGATGTGCCCTGTCAAGTTC
ATATGATGGAAATTTAATCCCCAATGCAATAGTGTTGGGAGGTGGGTACTAATAAGAGATGATCAGATCATGAGGGCTCCGCCCTCTCAAATGG
ATTAATGTGCTTACTGGGGGAGTGGATTAGTGACCTTGAAACCGCCATTACAAAATTGTAACTGAGACAGTGAAAGAGCTGACCTAACCAACTC
CATCTTGTTTCTAACCTCCAAGCTGTCCTTGTTCTTTGCTGGGCGTAGGCTGAACTAACTTTGGGAGGAACTTAGTTTGTAGATTAGCCCTTTC
CCAAAACAAACCTCCTTCTTGCCTGGGGACTAGACTGCCTTTGTGGGACTAACACCAGCCACAAGATTAGAAATTATGGTTTAGGAGCCATGCA
GCTGGAGGCTACAAGATTCCGACCCTCCCTAAACTGCTCCTAAGATCAGTGCTTGAGATATTCTGCAGACCCTGCACTTGATGATCAGCTGGCA
CCACCCAGATTGATAAACTGGCTCATCTGATCCTGTGGCCCCCACCTAGGAACTGACTCAGCACAAGATGATAGCTTCGACTCCCTATGATTTC
ATCTCTGACCAATTAGCACTCCTGGCTCACTGAATTCCCCACACTCACCAAGTTGTCCTTAAAAACTCTGATCCCCAAATGCTTGAGGAGACTG
ATTTGAGTAATAAGAAAGCTCTGGTCTCCCGCACAGCTGGCTCTGCATGAATTACTCTTTCTCTATTGCAATTTCCCTGTCTTTATAAATTGGC
TGTGTCTAAACAGCAGACAAGGTGAACCCACTGGGTGGTTACAACCTCAGGAGTGAGTGGGCTTATTATGAAAGCGAGTTAAGCAGCCTTTCTC
TCTCTCTCTCTCTTGCATCCTCTTCCTCTTCCACCTCAAGCTATGGAGTGACACAGTAAGAAGGCCCTTGCCAGGTGCAGGTCTCCCGACCTTG
GATTTCTCAGTCTCCAGAACTGTAAGAAATAAATTTCTTGGCCGGGCGCGGTGGCTCACGCCTGTAATCCTACCACTTTGGGAGGCTGAGACAG
GTGGATCACCTGTGGTCGGGAATTCATGACCAGCCTGACCAACATGGAGAAACCCCGTCTCTACTAAAACTACAAAATTAGCTGGGAGTGATGG
TGCATGCCTGTAATCCCAGCTACTCAGGAGGCTGAGGCAGGAGAATCGCTTGAAACCGGGAGGCGGAGGTTGCGGCGAGCTGAGATCGCGCCAT
TGCACTCCAGCCTAGGCAACAAGAGCGAAACTCCGTCCCAAAAAAAGAAATAAAAATAAAAATAAAAAATAAAAATAAATAAATTTCTTTCCTT
TATAAATTACCCAGTCCGTGTCATTCTATTACAACAGCAGGGAAGAGACTCAGATAGGAGCCCTGGGGCATATCTTGGCTCTGCAATTCACCAA
CTCCATGGCTGAGGGTCAGTCACTTCCCTCTGAATCTCACTGTCTCTCTCTAAAACAACAAGAATAATCACCACCTCACCAGGTTCTCATAAGG
ACCAAATGACGTATAGGAGGGAGGTGCTAGCACTTGAAGATGGGGCAATCTTTTAAGAACCCTCATTGCCCTCGATATTGACACATTCAGCCCT
TGTCCAGCACGGCCCACCCATTCCACTGTTTTGCTTTCTGGATTCATCTCTAAGTGGGTTTCCCCAGGTGGGAAACTCCTTTAAAGACAATTAG
CATACCTCTGGGTGCTCCCCTCTACCTAGAACACGGCTATGCTCATAATAGCTGCTTCATAATGTTTGTTAAATAATGACAACCAATGAAAAGA
CTTGACTGATTGTTCAAAGCCAGATAGAGAGACTCAATGTGTGCAGACAAACTGGTTACACAGGCTTGTCTCATCTGGAACTATGGAAAACTAT
GATTAATCTGGTTTTTAATTTGGTTACTTGAATGAAATAGAATATGGCAACAGACATGTAACACCCTGAGTTTTTGCTTGGGTTGGTTTAGCCA
CTTTTCTTTGGCATAAAGCCAGGAAGAACAAGGAATTTAAACTAGAGATTTGAAACCAAGGAAGCTTTAGTTAGATGCATCTTTGTTGGTAAAG
AGGGCAGAATTGAGACCTGAGTTTATTATTATAAATAGC
HUMAN SEQUENCE - mRNA
CTCCGGTGTGTCTGTCGGTTGCAGTGTTGGAGGTCGGCGCCGGCCCCCGCCTTCCGCGCCCCCCACGGGAAGGAAGCACCCCCGGTATTAAAAC
GAACGGGGCGGAAAGAAGCCCTCAGTCGCCGGCCGGGAGGCGAGCCGATGCCGAGCTGCTCCACGTCCACCATGCCGGGCATGATCTGCAAGAA
CCCAGACCTCGAGTTTGACTCGCTACAGCCCTGCTTCTACCCGGACGAAGATGACTTCTACTTCGGCGGCCCCGACTCGACCCCCCCGGGGGAG
GACATCTGGAAGAAGTTTGAGCTGCTGCCCACGCCCCCGCTGTCGCCCAGCCGTGGCTTCGCGGAGCACAGCTCCGAGCCCCCGAGCTGGGTCA
CGGAGATGCTGCTTGAGAACGAGCTGTGGGGCAGCCCGGCCGAGGAGGACGCGTTCGGCCTGGGGGGACTGGGTGGCCTCACCCCCAACCCGGT
CATCCTCCAGGACTGCATGTGGAGCGGCTTCTCCGCCCGCGAGAAGCTGGAGCGCGCCGTGAGCGAGAAGCTGCAGCACGGCCGCGGGCCGCCA
ACCGCCGGTTCCACCGCCCAGTCCCCGGGAGCCGGCGCCGCCAGCCCTGCGGGTCGCGGGCACGGCGGGGCTGCGGGAGCCGGCCGCGCCGGGG
CCGCCCTGCCCGCCGAGCTCGCCCACCCGGCCGCCGAGTGCGTGGATCCCGCCGTGGTCTTCCCCTTTCCCGTGAACAAGCGCGAGCCAGCGCC
CGTGCCCGCAGCCCCGGCCAGTGCCCCGGCGGCGGGCCCTGCGGTCGCCTCGGGGGCGGGTATTGCCGCCCCAGCCGGGGCCCCGGGGGTCGCC
CCTCCGCGCCCAGGCGGCCGCCAGACCAGCGGCGGCGACCACAAGGCCCTCAGTACCTCCGGAGAGGACACCCTGAGCGATTCAGATGATGAAG
ATGATGAAGAGGAAGATGAAGAGGAAGAAATCGACGTGGTCACTGTGGAGAAGCGGCGTTCCTCCTCCAACACCAAGGCTGTCACCACATTCAC
CATCACTGTGCGTCCCAAGAACGCAGCCCTGGGTCCCGGGAGGGCTCAGTCCAGCGAGCTGATCCTCAAACGATGCCTTCCCATCCACCAGCAG
CACAACTATGCCGCCCCCTCTCCCTACGTGGAGAGTGAGGATGCACCCCCACAGAAGAAGATAAAGAGCGAGGCGTCCCCACGTCCGCTCAAGA
GTGTCATCCCCCCAAAGGCTAAGAGCTTGAGCCCCCGAAACTCTGACTCGGAGGACAGTGAGCGTCGCAGAAACCACAACATCCTGGAGCGCCA
GCGCCGCAACGACCTTCGGTCCAGCTTTCTCACGCTCAGGGACCACGTGCCGGAGTTGGTAAAGAATGAGAAGGCCGCCAAGGTGGTCATTTTG
AAAAAGGCCACTGAGTATGTCCACTCCCTCCAGGCCGAGGAGCACCAGCTTTTGCTGGAAAAGGAAAAATTGCAGGCAAGACAGCAGCAGTTGC
TAAAGAAAATTGAACACGCTCGGACTTGCTAGACGCTTCTCAAAACTGGACAGTCACTGCCACTTTGCACATTTTGATTTTTTTTTTAAACAAA
CATTGTGTTGACATTAAGAATGTTGGTTTACTTTCAAATCGGTCCCCTGTCGAGTTCGGCTCTGGGTGGGCAGTAGGACCACCAGTGTGGGGTT
CTGCTGGGACCTTGGAGAGCCTGCATCCCAGGATGCTGGGTGGCCCTGCAGCCTCCTCCACCTCACCTCCATGACAGCGCTAAACGTTGGTGAC
GGTTGGGAGCCTCTGGGGCTGTTGAAGTCACCTTGTGTGTTCCAAGTTTCCAAACAACAGAAAGTCATTCCTTCTTTTTAAAATGGTGCTTAAG
TTCCAGCAGATGCCACATAAGGGGTTTGCCATTTGATACCCCTGGGGAACATTTCTGTAAATACCATTGACACATCCGCCTTTTGTATACATCC
TGGGTAATGAGAGGTGGCTTTTGCGGCCAGTATTAGACTGGAAGTTCATACCTAAGTACTGTAATAATACCTCAATGTTTGAGGAGCATGTTTT
GTATACAAATATATTGTTAATCTCTGTTATGTACTGTACTAATTCTTACACTGCCTGTATACTTTAGTATGACGCTGATACATAACTAAATTTG
ATACTTATATTTTCGTATGAAAATGAGTTGTGAAAGTTTTGAGTAGATATTACTTTATCACTTTTTGAACTAAGAAACTTTTGTAAAGAAATTT
ACTATATATATATGCCTTTTTCCTAGCCTGTTTCTTCCTGTTAATGTATTTGTTCATGTTTGGTGCATAGAACTGGGTAAATGCAAAGTTCTGT
GTTTAATTTCTTCAAAATGTATATATTTAGTGCTGCATCTTATAGCACTTTGAAATACCTCATGTTTATGAAAATAAATAGCTTAAAATT
HUMAN SEQUENCE - CODING
ATGCCGGGCATGATCTGCAAGAACCCAGACCTCGAGTTTGACTCGCTACAGCCCTGCTTCTACCCGGACGAAGATGACTTCTACTTCGGCGGCC
CCGACTCGACCCCCCCGGGGGAGGACATCTGGAAGAAGTTTGAGCTGCTGCCCACGCCCCCGCTGTCGCCCAGCCGTGGCTTCGCGGAGCACAG
CTCCGAGCCCCCGAGCTGGGTCACGGAGATGCTGCTTGAGAACGAGCTGTGGGGCAGCCCGGCCGAGGAGGACGCGTTCGGCCTGGGGGGACTG
GGTGGCCTCACCCCCAACCCGGTCATCCTCCAGGACTGCATGTGGAGCGGCTTCTCCGCCCGCGAGAAGCTGGAGCGCGCCGTGAGCGAGAAGC
TGCAGCACGGCCGCGGGCCGCCAACCGCCGGTTCCACCGCCCAGTCCCCGGGAGCCGGCGCCGCCAGCCCTGCGGGTCGCGGGCACGGCGGGGC
TGCGGGAGCCGGCCGCGCCGGGGCCGCCCTGCCCGCCGAGCTCGCCCACCCGGCCGCCGAGTGCGTGGATCCCGCCGTGGTCTTCCCCTTTCCC
GTGAACAAGCGCGAGCCAGCGCCCGTGCCCGCAGCCCCGGCCAGTGCCCCGGCGGCGGGCCCTGCGGTCGCCTCGGGGGCGGGTATTGCCGCCC
CAGCCGGGGCCCCGGGGGTCGCCCCTCCGCGCCCAGGCGGCCGCCAGACCAGCGGCGGCGACCACAAGGCCCTCAGTACCTCCGGAGAGGACAC
CCTGAGCGATTCAGATGATGAAGATGATGAAGAGGAAGATGAAGAGGAAGAAATCGACGTGGTCACTGTGGAGAAGCGGCGTTCCTCCTCCAAC
ACCAAGGCTGTCACCACATTCACCATCACTGTGCGTCCCAAGAACGCAGCCCTGGGTCCCGGGAGGGCTCAGTCCAGCGAGCTGATCCTCAAAC
GATGCCTTCCCATCCACCAGCAGCACAACTATGCCGCCCCCTCTCCCTACGTGGAGAGTGAGGATGCACCCCCACAGAAGAAGATAAAGAGCGA
GGCGTCCCCACGTCCGCTCAAGAGTGTCGTCCCCCCAAAGGCTAAGAGCTTGAGCCCCCGAAACTCTGACTCGGAGGACAGTGAGCGTCGCAGA
AACCACAACATCCTGGAGCGCCAGCGCCGCAACGACCTTCGGTCCAGCTTTCTCACGCTCAGGGACCACGTGCCGGAGTTGGTAAAGAATGAGA
AGGCCGCCAAGGTGGTCATTTTGAAAAAGGCCACTGAGTATGTCCACTCCCTCCAGGCCGAGGAGCACCAGCTTTTGCTGGAAAAGGAAAAATT
GCAGGCAAGACAGCAGCAGTTGCTAAAGAAAATTGAACACGCTCGGACTTGCTAG

TABLE 6
MOUSE NOMENCLATURE
ICSGNM Myb
Celera mCG2825
HUMAN NOMENCLATURE
HGNC MYB
Celera hCG3238O
MOUSE SEQUENCE - GENOMIC
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNGTCCAAACCTCTTTGTTTGATGGCATCTGTTTACAGTAGTTACACTTTAATATCAACCTGTTTCCTCCTCCTCCTTCTCCTCCT
TCTCCTCCTCCTCCTCGGTGACCTCCTTCTCCTCCCCTTTCTCCGGAGAAACTTCGCCCCGGCGGTGCGGAGCGCCGCTGCGCAGCCGGGGGAG
GGACGCAGGCAAGGCCGGAGGGCAGCGGGAGGCGGCAACCGGTTGCGGTCCCCCGGGGCTCTTGGCGGAGCCCCGGCCCGCCTTCGCCATGGCC
CGGAGACCCCGAACACAGGTAATGGGGAGGCTGAGAGGGCGGCCTCGGGCTAGGGCGCGCGGGGATCCAGGCATCCCGGAGCAGGTGGGAATGC
CTTCCCTGTTGGTTTGCGGGGCTGTCAGCCAGTGGCAGAAGACACTCGGGAGAAGGATGAATGGAAAACTCTGCGAGAAGCGCACGGGTATAGT
GGGAAGCATGCCCTGAGGCTCTTTTTTATTTTATTTCCCTTTTTCTTTCTTTTTTCTTTTTCTTTTTGCAAGTTGCTCGGGGATTCGTTTCTCT
TTGGGGGGTGGGGTGGGGGGGGGGCTCGGCACTCTTCAGCCGAGGACTGAACGGTTCCATCAGACCCGAGGAGCCCACCAGGTGTTTGGCTTGA
TGCCCCGAGTGTCGGCGAGGGAGTCGCAGCCTAATTGCTGCATGCATTGAGGTGTGTTTGCACTTGAAGAGCATATGGCAGATATAGGGAAAGG
CCGGGTTGTTTAGTAGCTGCAGGTAGTTCATTTAAATTTCATTCATTCATTATGGAGGCGAGAGAGGTGTCAGCTGACAGGCAGGCAGCTGCAA
AGATTTTAGGGGGACAAAATGGGTTTGCAGATTGTTTTAAACCCAGCCGTTTGAAGATTCTTGTACTTGTTATGGAAGAAGCTTTTGCAAACAC
TGCACGCGCCCGCGTCGCACATGTCACCGCACCGGCCGAGATGATGCGTGCACTCTCTTTCCATTTTGGCCCCACCTTCGGGAAGAGGGATCCT
CTCGGAACTGTCAGTTGCTGGCGCTCAAGTCCGGCCCGACTCGGGGTTTCCCGAGGACACGCCGGGGCTCCCAGAGAGCAACAGATGCGGGGCT
TAGCTCGGTGGCGGTCGCCACTCGGCGCAAAGTGCTGGAGGCAGGCGGCACCCAAGGCAGCTGCAGTGCACCCCGGGCGATGTCGCCGCTCTTC
TCCTGGGCCGCGCACACGTGGAAGCAGCTGAGGTCGCCGCTGCTCCCAGCACTCTGCTCCAGCTGCTTTTGGTGGCCCGCCTCTGGCAAAGCCA
CCGACCGACCGACCCTAAGGAAGNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNGGTGAATTGGGGAGACCTTGAATCTTGGCTCCCAGTGC
CTGGCCCCGGGCCTGCCGCGCCAGCCTAAGTGCCACCCTTTAAGAGGTTTCGCCTTTCGCTTTTGGGACCTAAACCGCCCGCACCCAGGTCACC
TGTTCTGCAGAAAGCTGAAACCTGCAGCAGGAGGAGGGAGTTAGGAAGGGGTGCTCCCAGGAGGTGCGGCCCAAGCTGGCCGCCCCAAGGTTCA
GTCGGGGCGTGGCCGCGCAGGTTCCCTGGGGCTCGCAGGTAGCCCGGAGCGGAGGCGGCAGGGAGGGGCGGGAGCCTTGAACTTGACTCAGGGG
GAAGAGAAACCCGGCTGCGGGCTCGGAGCCGTTTCTTGCAGGGCTAGCGTCAACTCGGCAGGGATTATTAACCAGGTCAGCGAAATGACTCTGA
ACCCCTTTTCTAGAATCACGTAGCGGCCACAGGCACTTCCTCCGGTCCCCCTCCCTCTTCCCAACCCCCCAGTGTTCTTTTTCGTTTCTTTGCA
CGGTTTGTTTCCTTGATAGAATGTAACATTCATAGTTGTACTCTTGTAACCACACCGGCTCTCGCAGGTGGAGCAGTCGAGAGCATCTAGCTTG
AAATCAGCCTTTGAAAGTCCTTTGACTGTTGGGTCTGAGATCTTGTCGGAGCAGATAAAGGAAAAGTTATTTCGGGTCCTTTGTCGAAATTAAT
CATGCTACCGAGGCGACCAGGTCATTTTAGAGCAAGGAAATGGCATGCAGAGCTCAGAGGCAAGCCCCTTGTCAAAGCTGACAGAATCCTGGAC
TCGCCCAGCTCCCTCTCACAGCCTTTCAAGATCTGCAAGAGTCACACGCCACATTTCACCCCAAGACTTTGGCTTTGCACATCTGCCCAGGGCT
TGCTTTTTAAGAGGCACAGAAGACAGTAGAGGCATGCCTCTGGAAAGTACCTTAAACATAGAATCCCCTCCCTAGTGTGTAAGATGGGATCCAC
GAGAGGGAGGGGGGTTTGTCCAAATCAAACGACTAGGTGCGTGGACAACTAGGGCTGTTAGAACTTTGCTCCTAGACCCCCTGGTCCTGTGTGA
TCCCAGTTCTCCCTCACCTGACTATTGACTGCCCCCTGTGGGCGACTGAGGGTGATGCTTGTTTTTAGATTAAGTTTTGACCAGAGGAAAATTT
GCTTGGAAAATGTCCACAATGCTGAAAGTGACACCTCCAGATCTGGACAAGCCTAACAGGCGAATCTAATGAGAGTTAAATCGGTGGCTCCACT
CTCAGGTCCCGTTGGCTTTCTTGGTATCCCTGATACTTCACAAAGGGATGTTTCATCCTGAGAGCTTTCCAAGTTCCTTAACAAAAGGGAATTG
TCACCCAAAGAGGCTGGTTTGCAGTTCCGGCCTCATAACTCTTTTTCCCTTCACAACACCATGTTTTATAAAATCGTAATTATTACTGCACAGA
AGTTATTTGCCGCCTCCTGTTTACTGTTGGAAATAAAACTGCTTTTGGTTGAAAGGATTTACACTAAGTGGAAAAATATATTGCAGATCATGAA
TAAAGTTTCCTGGGATTTTTTTTTTCTTTTTGGTGGTGTTATTTTAACGTTAGATCACATCCTGTCACCATGAAGCCTGAGCCATCAGTTTGAG
GCTCAGAATAATATTTTAGATGATGAAAATCCCAATGTTTGGTAAACACTTTGGCTTAACTTTTAGGGGGCCAGAAATATACATTTAGTGAGTT
CGTCTGGAAAGACAAATCCTATTATGAATGTAAGTTTGCCTTTTAAAAATAATTTTCATTATGATGTTGTCTTATATCATCTTGCTGGTTCGAT
AGGTTTTTCAGTTTTACAACAGTGAGTTTGTTAAGTGGAGTTGTTAAGTGTAAAATTCTTCACATTTTAAGTCTTATAGAGTGATAACAAAAAC
TAAACTGCAGAATTACTCTGTTCTGTATATGATTGATGAGTTTGGAGTTCCTTCAGCTGCCATCAAAATTCACACTGTTACTCAACCCGGTAAA
AGAAAAGGCTGTTTTGAAAACAGTCTTTTTTTTTTTTTAACCTGTAGATTTCATCTGGGGGAACCCCTTGGTTTTTAATTAGATACATTGTAAG
GTATTTTAGACTCCATGCACTCAGAGACAGAGAAAACAAGAGTAAGTAGGAGTCTGAATAGCCAGTGCCCTGGCCATGTGCTTAGTTACTCAGG
AGACAGCACTGTAAACACAAGCACAGCTTAGGTAGAGTAGACACGGGCGGGCAGCGTTTGAATGATTTGCAATTTATGTTAATCACAAAACAGT
TTGAGGGATGTCTCCTTTACCAGGTCTTGAAAATCTTCCTGATTTTTTTTTCCCCTAGTGCCATGGTCTTTAAGAGGTGATCACAACTATACTT
TGGAATGTATTCTGTCTTGGTTTTTTTATTTTATCAGACTACCAGTACTCTTGAGACACACTTGATTGGTCTGTTGTGTTCAGAAACTATTTAA
ATGATTTGCTATTTGCTATATGCCAGAGTTGGAGTGTGTATTTATAGCAAGGACATGGGCTCTTGTTCTTCTCATTGTCCTTAACATTGACTTT
TAAACTCAAGCATCAATTGGTTGGGAGTTTGCCAGATGTGTAACTTGTCACTGATCTAACAGTAACAGGTTCAAACATGCAGAGCAATAGAAAA
ATGCCAGGGCTCTTGGTGTCTCTGTGGATACTAGTAACAGCAAAACCAGTTTACAAACACTACAGCACCAGAATGCAGAAAACCTCATTGCGCA
ACTGTGCAAGGTTTTGTTTCTGTAGACTTTTAACAAGCTTGCTGAAGCGTTTCTGTCTGAGTTTCATAATCCAATAGTTTAGATCCTCTGCCTT
TGGCCCATGTGTGTCCATTCTTATTTCTGCAGCATCTACAGTAGCGATGAAGATGATGAAGACATTGAGATGTGTGACCATGACTACGATGGGC
TGCTGCCCAAATCTGGAAAGCGTCACTTGGGGAAAACTAGGTGGACAAGGGAAGAGGTAACTAGTCATTTTATCAAAACGTAGGGAAACTCTGT
AGAGTCTGTGATTGGTGAAAACTGAAATCTCGGCTAAGCTACATGTGCCCTTCTGAAGACTCTTGAAGAGGTTATCTTGGCATGCTGTACAGAA
TGCTCGTGTTCTTCTCTCCCAGTGTTTATTCTGCTTTCTGTTCCCAACCTGTTTATTGTTCCTTAAATGCTTTCTCCTTTTTCCTTTCCACTTT
GCCTGTCTGGTTCTCATTCTGGAACCTCACCATTCCCTACACATTGCCTTTTCTGTGAAACTGTTACTGGCCACCATCTTTAAGGCTGGATGCA
CCCTCCCGTGGTCCCAACCCCCCTGGGCACATGCTTGGCTTTATTGGTGTTGTTACATATTTGTCTTTCCCAACACACTGCCAATTCCTTGAGG
GTTAGGACTAAAGTATCTCTTCATCTTTGTGTTCCTCAAACTCAAAATTACTTGCTAAAATGTAGGATAGTAATGAATGTTCTCTGGCTAAAGT
GATGCTTTGCTATTTTCTAAGCTAAAGTAGCCGTAGAATTTATTGTGTTGTAATATGCTCAGTGAACATTCGTTACACATGATAGTCACATGAC
CTATGTATGTGAAAGATTATTCTGAAGAGATCCAGTAATAGGAGAAGATATGCCCACTTCTTTCTAGAGCAAAAATCATCAAAGTATGAAGAAA
GCTTTCTTCCTTCTCTACTGAACTGTGACCTTGATTGCATGCTAAAATCAAAGCTCCTGAGAACAGGTGGAAGCACTCCCAGGGAGGCACTTAG
GGAATGCACGTTCTGTCTGTCTGTCCTGCTCAGGCCCGTGGATGCTCCCTTGGAGTGTGAACTTCTGTTGTTAACCATGGATTCCCAGACTCTG
TCTGTTTTGTCATTATGTGTGAATGCAGAAACCTATGAAGACTGTGTTTACAAAGGTGTGCATGAGCTAGCATGTAGATGAGCGTATGTGCTGA
TGGAGCCAGCTTGGGAATGTCTAGCTAGTGTCTTCTGTGTTCTCATCCATAAACTCTGAACATATTGATGCAGATCCTTAATGGCTTTACTGCA
TGAAGTACACCTTCAAGTCAAATGTGTCAAATATCTTCTTCTTTGCATATGGAGAAGGTTGAACCAATGGGAAGCTGTCTCATCCATGGTCTTA
TCTTTCTCAGGGTTGAACGGTGACTAAGTTTAACGTTGGAATTCATTTTCCCCCATTTAGTAAGAGTGTTAACAGTTACCATCTATTAAACACT
AACACATGCCCGGCTTCACTGCTTTACATAATGGTATTGTGTAATCTCCAGATAACAATGTTGATACTACCATTCCTATCTTATAGATGACAAT
AGAAATCTGTTTATATAACTACTCAACAGCTGTGTCAAAGTAAAAGCTTTAGTGTGCCTGTGAGTAAATGTATGCCCAGACCAGTATATTACAT
TAATATTGGCTTTATTTGAGTGGTTTTGAAAGCTCCCCCCCACCCCCGATCATGAGGTTGGTTCCTTTTTTCATTAAACTTAGACAGAGCTGTT
CAGTAAACAAGTGATGCCTAGGATTGTCTTTACACACCTTAGGATGAGAAGCTGAAGAAGCTGGTGGANNNNNNNNNNNNNNNNNNNNNNNNNN
NNNNNNNNNNNNNNNNNNNNNNNNNNNNNNNNNNNNNNNNNNNNNNNNNNNNNNNNNNNNNNNTCTTTCGAATGGAAAAATCTCATCTGGATTT
AAGAAATTATTGGATAGTTTACTAAAATACTATGTATTTGCTCATATAGAGTTAAGAGGAAGAGAGCCATCCGTAATTTTTATATCTGACTACA
CAACATATAATTGAGGTTTCTAGAGTTACTATCCAGAGATTTTGATTTTTTTTTTCAACAATTGCTTCTTGTTTTGTTTTAAAATACCCTGATA
GTGTAAGTTAACATTCTGATGGGTTGTTCCAGGATAAAATTTTAAACTTGGTATTCCCTCCAAGTTAGCTTCTAAATTCTCCTATTCTCATCAA
CAGCCACATTTTTGGTGTGTGGGTTTAAAAATCTAGAGCGTCTTTTGTCTTATAGCCTCCCAGGTCCATCTCAATGATGAGTCGGGAGCGCAGA
ATTTCTTTACAGTCCTTCTCTTCCACTTCTTTCCTATGGTCTGGTCACAGGGCAGCTGCCCTGCCCTCTCTATCATAGTGGCTTCCTTCTTGGC
TTCACCATCTTGGCTTCACCACGTGTATTCTTTGAGCTTACCACCAGTTCCAAACACTGCTAGCTGAGGGAGCCTCCTAAACGGCAACTACTCC
TAACTACTCTGCTTGCCTAGGGTGTCTGAGTCCCTAGGTTTGGTCTCCTGCACCAATAAAATCAACAAATAAGCAAACAAACCCCCAGCAGTGG
TGACATAGGTTCTCACATGGCAAGTGGGCTACCTTGACCTGGCATCCAGTTTCTCTCCTATCTGGTCTCAACTTACAGTCCCACTGCCCTCTCC
CTCCTCTACCCGCTCTGTTTCCTGCTTCCTCCCCACAAGCAGGTTTGGCTGTGTTCATTTCTGCTTTTTCTCCTCTGGGCTTTTCTTCCTGCTG
ATTTCACCCCCACCCCCCACCCCCAGCATGTTCTCTCTTGCTTCTCCAGTTAAAAGTACTGAGCACCCTCCAAAGGCCGGATCATGAGCTCTCG
AATGCCTCTGAAGGAAGCAGAACCTCTTCTTTAAGAGTCCTTTTCTCCTCACCCCAACAGCACGTGGCTTAGGTCTCATCTTACAGATAAAGTC
AGCCTTTGGTGTAATCAGTTACATGTCCATCTTTCTTCTTCTAAGATTATGTCTAAGAAAGTGAGAAAGTTTTATTGTTTTCATTCAGTCTAAT
ACAAGTGTCTGTGGTCCTGAACACACAGAGAGGTTGAATGGGAGGCACTTCAGTTCCTTAGGATGGATTTTTATTGAGGCGTGGCCATGTGGGA
TCACTAATGCATCTTCAGCCACAGAGGTACAAACCACAGACTGAGGTTAACGATAAGTCACGTTGGCATCTATGTGTGTACTGTGATGTTCCTT
CTATGCACATGGCAGACCATTACCTTCAGGCTCCAATCTTAGATGCAACTTGGATACATTCTCTAGTCCGTGAGCAGTGTGGGATGGATAGGTG
GGCTCTCTGTACAGGCTTCTAAATGTTCTTAGATGTTTTCACTATGTATCATCCACATGTGATCCCTATTGAACAAAGCCTTCATGTGTTCTTC
ACGCCTTATCATCCCAACGCTGTGTACTTTGTCTAGAACCGGACAGATGTGCAGTGCCAACACCGGTGGCAGAAAGTGCTGAACCCTGAACTCA
TCAAAGGTCCCTGGACCAAAGAAGAAGATCAGAGAGTAAGTTCTGTCCCCATAGATGTGTGACTTGCAGTTAAGAATATTGCTAAAACAGGTTT
CTCACCCCAAGGAAGCAATTTAAATCTTCAAACAGGAAGTAAAGGGCTGGCTCCTGCATGTCCAGTGGATGAAAGCAGATTATTTCAAGATCTG
AAGATCTTGTAACATTGAGGCTTGATGACGCTGACTCTTTCCGTAACATTAAAACACAGATTATTTTTCTGTTCATCTGAAGGTCATAGAGCTT
GTCCAGAAATATGGTCCGAAGCGTTGGTCTGTTATTGCCAAGCACTTAAAAGGGAGAATTGGAAAGCAGTGTCGGGAGAGGTGGCACAACCATT
TGAATCCAGAAGTTAAGAAAACCTCCTGGACAGAAGAGGAGGACAGAATCATTTACCAGGCACACAAGCGTCTGGGGAACAGATGGGCAGAGAT
CGCAAAGCTGCTGCCCGGACGGTAAGACTTGGGCAACAAATATATTGATTGGGAAGAACCAAGGGATGGTCGCTAAACATTCTCTGTGTGTGAA
TGAATTACATTCGAGCAATTATAAGCATTTTGTCAGATAAACCAGAAACACCAACTTTTAGGAGCGGGCAAGAGCTTGGAGTTTGTTCCGTTGG
ACAAGGCTAGTGACACCTGGGTGGTGAGCCCCAGCCAAACCTTTCGGCTTTCCTCCACTGTTGCTCCTCCATTTGTGAAATAGCTGATTTCCAT
GAGTTAACGTTAGGATCAAGTGAAATGAATGTCAGGTATTCTGTAAGCTAAAGCACCTACCTGCCTAAGAAGGGAGTGCAGACACAGATTGCTC
TCGGGCTGAGGCCATCCAGCCAGTTCAGCCATTACTGGTCACTCCTCCACTTTAGCAATTTCCGTTTTCCTAACACCTGCATTCACCTCATTTC
CCATTTGATTACTGGGTGTTTTTTTGGTTTTGTTTTTCTGAGACTAAAAAAAGCCTGTATGTCTAGAGTTGTCACGACAACTTCAGGTTTTTTA
ACTAATCTCAGTGTTATGTCTTATTTTTACCAAACAAGGAGGGGAAATGACGCCTTTGTATAGGATAATTATGGAAACTCACCTTTGACCCACT
GTCTACACATATATACATTGATTTTTTTTTTTTAGCTATTTTGAGAAATACTTTCACTGTAAATTTTAAAATAAACAATATTAAAAGTATTCTT
ATTCTTGAGCTTTTTGACCCCAAAGTGGGGAAAGGATTGTTTCAGGATGGCTTATCTAGTATTTCCTAGTCTTGCATCAATGGGGTAGCTCCCT
CATGCCTTGAGAACTTTAAGAAGCCATGGATCTGGTCAAAGGAGGCCTTTCCTCCCATGTGGCCTCAGAGGATATACTGTTGACTTTTCTAAAG
ACTAAAGGGGCAACTGAACTTCCTCTTTTTTAAAAACACACATTTCCCCACCCCCATGGAAATAGTAAAAATGTTTTTACTTAATTTTTCTTTC
TTTTCCTTTTCTTTTTCTTTTTTATTTTTTTAGCTAAGAAACTTCCTGAAAATGGGAAGAGTTTAGTAAAAGCACCTTGCAGTTTGAAACTATA
CTAGGGGGTTGAATGTGGTAGCACATGCCTGTAATCCCAGCANNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNCTAAGCCACAATGGAAGGGCTTTATAACAGT
GGGGACTCTAAGTTCTATTAAAGTCCAGTGGCACGTGAATTCTGAGGACCTACGCCTTTATCAAGAAATCAAAATGCTAAAATAAATTTTGCAG
GAAACTTGATACATCCCAAATCAGGTGAGCAAGAGAAGAGTGGCTTTTCAGTTAGGCGACAAGGTAGAAAAGATGAAGACAATTTAAAAGAGGG
AAAGAAAAGTTAGTGGAACCACAGCAATTAAAATGAGAAAATAAAAGTGGAACCACAGAAATTAAAGTATTTTTTGTTTGTTTTTTAAGAAACA
GTTTTCTGTTGATTGACTGTAATCTGAGTTAAAAGTTTTATAAAGTTTGTATATGGTACATTTCTACACAGGACTGATAATGCTATCAAGAACC
ACTGGAATTCCACCATGCGTCGCAAGGTGGAACAGGAAGGCTACCTGCAGGAGCCTTCCAAAGCCAGCCAGACGCCAGTGGCCACGAGCTTCCA
GAAGAACAATCATTTGATGGGGTTTGGGCATGCCTCACCTCCATCTCAGCTCTCTCCAAGTGGCCAGTCCTCCGTCAACAGCGAATATCCCTAT
TACCACATCGCCGAAGCACAAAACGTAAGCCACTCCCGAGAATGTAGAGGGAGAAGAGAGGTGGTTATATCACCTGGGGTCATAAAAATACTTT
ACAGTTGTGTGCTTTGCATAGATGTTGTACAAGCATGGTCACAGATGGAGATGTGTCGATGGCCCTCAGCTAGACCCACTGTGTGAGGTAGGAG
ATGGCCCTCAGCTAGACCCACTGTGTGAGGTAGGAGATGGCCCTCAGCTAGACCCACTGTGTGAGGTAGGAGATGGCCCTCAGCTAGACCCACT
GTGTGAGGTAGGAGATGGCCCTCAGCTAGACCCACTGTGTGAGGTAGGAGATGGCCCTCAGCTAGACCCACTGTGTGAGGTAGGAGATGGCCCT
CAGCTAGACCCACTGTGTGAGGTAGGAGAAGATACACTTGGCTTCCGTTTCTGGAAACAAGAATGAAAAGGAATTTGTCCATCACATTGAAGTA
GTGCTGAATAGAACCAAAGTCGTCTGATTCTAGTTTTCTGCTCCAAACCTGATTTAAAGATCGTGTTTTAATGTAAATGTACTTGTATATTGCT
AAATCTTAAGAGTAGGTGAACATAGATAGATGCATAGTCTGTTAGCTTTCTTTTCCAAAATGAAAAATTGATGTAATATTACATTTAAGGTCAT
AAAGAGGAGATAGCTAACCTCTGAAACATGAAAGGAGATTATAAGTTCAATTCTGGCAATGATCTGAAGAAAATGAATTGATTTATATATCCAG
GAAATGTTTACTTTCTTACTCAGAGTCAAGGGCTGTACTGTGATATTAATTTGGTGATGTGCTCCTTCATCCTCGCTTGGGTTGGTATCATTTC
TTCCTCATTTCTAGACATTCATATCTAGCGAAAGAAATCGCTTACAGTACATGGGAACAAAGTCTAAAGAGGAGAATATCAAGAAATGCTTATC
ATCGAGCTGATGCTAAAAAATGACAAAACTCACCCAAGAATCACAAAGACCATATGCAAATGTTATGTAAAACATATGCATGTGTAGTTCTCAC
GTGGCTCTATCTGTTTCTTAGATTTGACTACAGAATTTCCAGAAAGGTCTCCAGTGAATGAATTATCATAAAAGTTTCTGTGCTTTTTATTAAA
AGGAACATTATTCTAAGAAAAGTACCCCTAGTGTACTTTTTTCACGCATCCCACGAACACTTTCCCGTTGTTCCTCTCTCCAACTGAGGGATGT
TTTCTGTTGGGATAGGCAGACTCTTTGGGCCTCTCTCCCTGTTTCTTGAACTAGTTAGCCATTTTTAAGTATCTGGCTAACTTTCAAGTTAGAG
AAGTTTTTGTTTTTGTTTTATTTTAATCATTCCATGAATTTTATCAACCAAGTACTTTCTCCCTTGGTCACAGAGCAAAATTTTAGAGAGCTGA
ATCCTCCTGGCGCACTTGTTAAGTAGGAATGACTTAGTCTTTGTTTTCTTTTCCTGGTCTAGATCTCCAGTCACGTTCCCTATCCTGTCGCATT
GCATGTTAATATAGTCAACGTCCCTCAGCCGGCTGCGGCAGCCATCCAGGTGAGATCATCTGACGGGCAAACTCCTTAGCTGAGCAAGGCACTG
CTTTTAAAGAATACTTTCTTTCTACACACACACATACCATGTAATAGAAAAGCTAACAGCATTGCCAACCTACTTTGGGCTAGCCTTTATTTAC
CCTTGGTGGCTTCTGGAATTTTCTGATGCATCCATGAGCAGGTGCTCTAGCCTCTGCCTTACCCCTTGTTGCCTTAAGTCCCGTATCACTTTGA
GCAGATAGTGGTGAATTCTGTGGGGTGATTCCACATGGAAGTTCTCTGGGCTGTGTAAGGTACCTCTCCCTCAGACCTTGTCTCTCCCTGCTTC
ACTTGAAACTCTAGAGACACTATAACGACGAAGACCCTGAGAAGGAAAAGCGAATAAAGGAGCTGGAGTTGCTCCTGATGTCAACAGAGAACGA
GCTGAAGGGACAGCAGGCATTACCAGTAAGACTGTGACCTTGGGATGGGTGGCTTCGCTCAGTGCTCTTCTCTCTCCTCCTCTGTCTCCTTTTC
TAAAGACTGTCTCATCCAAGTTCTGTGTGTCCGCTAACAATGCTCAAACAACAGAAACTGCTACTGATGACTGATAAAAGTCTGTTAGTCTCAG
AATGTGCATTGAGTGACTGGAAGTCACTGTTTTAGAATAGGACCACAGTACCCACTTGTGACTGTCAGGATCTCCATTGTAACAAAGCACAAAG
CTTTGCCTGCTTGTTTGATTCTTTTACTGTTATCCTTTTAACCTGGATTTACATTTCTTACAATCTCAAATTCCATTTGAAGTGTATTTATTAT
ATACATGTTTTTTAAAAGGGATCTATACATAAGCCACTCTTTAAATGTTTGCTATCTTTTATTTGCATGTATGAAAGCTCTCTGATCAGTAGTG
ATAAGGTGTTTGTGTAAACACATATATGTTCCCATAGATATGTGTATATATGCGTGCAGATTTCTGTGCATACATGTGTCCAGGCCAGAGGTTG
ACACCAAGTGTCTTTTTTGATAGGTTTCCACATTAAAAAATGTAGTTGTATGACTACCACTCATAAGGTACTATAAGATATATGTTGAAATAAA
CTCTTTCCTCTCAAAGAAAAAAAAAACACTAGTTGTAATTGTGTATGCATGTGTACATGTGCTTGTGTGCACTCTTGGAGGTAAGAGGACACCG
TTCCAGAATCAGTTCTTTCCTTCTACCGTGGGCTCTGGGGATCAAACTCCTTTTTAACTGCCTAGTCATCTTGCTGGCCATCCAGCTTATATTT
TGAAATAGGGTCTCATGAAAGCTGGAGTTTGCCCATTNNNNNNNNNNNNNNNNNNNNCCAGTGAGCTCGAGGGTGTACCTGTCTCTCCCTCTCC
AGTGCTGGGGTTACACAATGGAGCTGCTGTGGGTGCGTGGAGATCAAATCCAGGTTCTTACGATTGCATGGCAGTCTCCCTTCAGAGTGAGACA
ACCCCCTCCCCTTTTTTTTTGTGCTGACAATTGAGCCTAGAGTCTCAGACATGCTACATTGCACACACACACACACACACACACACACACACAC
ACAGGATCCAGCTCTAGCTCTTGTATAGACCGTTTCTGAATCCTTGAATGGGCAGCTCACGGAAAAAGCAAACTTTGGAGTTTTGGTAGTTTAG
CCAAGAAGTGAGTATGTGAAAGGTGATACCCACTAGCGATTCTTCAGGTGAAATGTTCTTTGTTGTTGTTATTAAATAATAAAATAATGTACTT
GTGCTAGTTCAACCACTTTTGAACTTTAGTTACTATTTTGCAATTTCTTCTCTTGCATGGACATGCTGTAAGGGGACTTCTGTTAACTTGATCT
ACTTTCCCAACCACATATACTCTGCAGCTCTAGCTTGCATCAGTGCAACCTATAGCCAAAAAAAAAAAAAAAAAAAAAAAAAAAAAAAAGAAAA
GAATAGAAGCAACAGGATATGTACAGATAGAAGCAATTCTAACCCTTGAATGCTTTCAAGGGCTTAACATGCAAATAGGACCATTCTTAAGTCA
TAGCAATGGGGAACTGTTTTTAAATTAAACTCCAATGGAATTCTAAGACATCTAAGACTTGTATTTTGTAGCAGTCCCAAGGTTTAACATTTGA
CTAAAATCTTAGCAACTGAAGCAATCCTCTAGGTATAAATTGGAGAGCTATTGCAAAGCTGTCAAAAAGGAAACATCTTGACAACTGTCTGGCA
GGCTTGAGTTTTGGATGAAGCAACCACTTTTTATCTTGCCTCCAACAGCATCTGATACCTTGTGCAACATAGTTGCTAAGTTTCTTCTTCCTTT
CTTCTCTCCATCCTTATTTCTACACACCCCCTTCCCCAGACACAGAACCACACTTGCAGCTACCCCGGGTGGCACAGCACCTCCATTGTGGACC
AGACCAGACCTCATGGGGATAGTGCACCTGTTTCCTGTTTGGGAGAACACCATGCCACCCCATCTCTGCCTGCAGATCCCGGCTCCCTACCTGA
AGAAAGTGCCTCACCAGCAAGGTGCATGATCGTCCACCAGGGCACCATTCTGGACAATGTTAAGAACCTCTTAGAATTTGCAGAAACACTCCAG
TTTATAGATTCTGTAAGTAGAATTGCTAAGGCACGTTGATGTTTCTGGAAGAGGAATTGAGAATTGGTATAGCATAAATCATTGCCATTTTCTA
TAGCAAATACGGTTTGGTCCTACATTCATGTAAAGTGAAAGACTTAGCTTTTACGATTCCACTTCTCTCAGTGGTCTTGATTTTGTAGGCTACC
CACCCAAAATTGTTTGCCTTCCAGGTGTGAATTAATGAAGTTCTGACTTACCACAAAGGGCTCAGAATGAGAGCTGTGTCTGAACTATTTTAAC
AAATTTCTTATATATTTTGCTTACTTCTTTGTGATTCTCTAGAAAATTTCTTTGTTGTTTTGGTTTTGCTTGCTTTTTGTTTTGTTTTGTCTGT
TTTGGGACAGCACATTTGGACTTACGTTTCCTCGTAACCTTAAGCTTTCAGTTCAAGGGTGGGGCATATAGATCTCTCTACAGCATACTGTAAA
TTGATTGACAATCATAGTAACACCAAAATAAACCATCTTGAACCATCTTGAGTGAAACAAATTTGAGTTCAGCAGTCACTCAGTTCATTTTCTT
GGTCTCCTAATTGGTTAAAAAGCACTTATTCCCTTCACACGGGACAGGGCCAGCTATTGCCATAATGTATCATCTAGAAATGTTTTCTGTTTGC
TTTAAATTGCATGCAAAAGAGAGTGCCTGAGCTCTCCACAGCAGATGTGCAAGACAACCCATGCACCGCTGTAATCCCTGAGCTAGAAAGCAGG
CAGGCAGGCAGGCAGGCCCAGCGTTTTCGTCCATTGCTCTTTCAGTGCTGTGTTTGGGTGATGGTGGCGCTGTCACTTGCATACCATCTCAGAA
GCTCTGTGCCTCCCACGTTGTTTCAGGATTCTTCGTGGTGTGATCTCAGCAGTTTTGAATTCTCTGAAGAAGCGGCAGCTTTTTCACCTAGCCA
GCAGCCCACAGGCAAAGCCTTCCAGCTTCAGCAAAGAGAGGGCCATGGGACTAGATCTGCAGGAGAGCCTAGCCTGAGGGTGACCAGGCGAGTG
CTGAGCGAGGCATCCCTCGGCCCAGACTCACCCCAAGCGAGGCACAGCAAGGTTCCGCTGGTCGTCCTACGAAAAAGGCGGGGCCAGGCCAGCC
CCCTAGCCGCTGGAGAGCCTAGCCCCTCCCTCTTTGCTGACGTCATCAGCTCAACTCTCAAGCGTTCCCCTGTCAAAAGCCTACCCTTCTCTCC
CTCGCAGGTAGAGCACAATTTCTGCACAGCTGTTACTAATTTTCAGCATACACCCGAGCCTTAATAATTAGAAACTAATCTAAATTTGAACAAA
TGACTAATGACTGCCGTCCTGGCAGATTTAATTCCGCGTTCAATCTCACTGGGGTAGAGGTGGCTAGCTTCCTGGCATTTGGGCTGACACACTG
GTGATTTTGTCGCATCTCTTTCTCTCGAAGTCTTGGTGTTTTGGAAAATGGACAGAAAGTTGCATGGGATGTGTTTCCATTTCCCTTTTCACTA
CATTTCCCTTTTTCCGTCCCTCCCCTCTTCTCCCTTGTCATCCTTGCCTCTTCACATTTTAACACGATGTCTGCCTCTTTGTTCATTGACTACT
AAGTGATTTACTGACCCTGGGGAGGAGGGAACCAAATTATGATTACATAACATTTGGATTACAACTAGAAAATGAACCACTATGCCCCTTTTCT
AAGTGAGGATGTTTACTGAGATGATGAAAAAGAGATGCTTCTGCAAAATAATTTAACATTTTCAACACTGGGTCTTTTAAATTCTGCTATGGGA
TTTTGCTAAGGTACCGTTTTCCAAAGAATACCCTTACTTCATATAGTGTTCTTAAGGTATATAACACTATAATGCTTAGCAGAGCCACGTGCTT
TCCAAATTATAATTAAGAGACAATTTGGCTCCTTAAATTTTTTTCCCAATTATATCCGTTGAAGATTTGGTACTTTGTTTTACTTTACTTCATT
TTAGTAGTAGTAGTAATAGTAGTAGTAGTAGTAGTAGTAGTAGTAGTAGTATTGTGGTTCTTAGAATCTAACCTGGAGCCTCATGTGTGCTAGG
GAAGTGCCCTACCACTGAGCTATACATATTTCTGGACTTGTTTTTAAAAGAACTTTAAGTATAGACAAATGATCCCCCCATTCCCCAAAACAAT
TGGTCTCCACCCCCCACCCACCCCCATATTTTCCTAAACCGTGTTTTGTGAAATCATTTCTGCAAGATAGATGTCTCAGGGAAAAGATCTAGG
TAGACATAAACGTTTGGGAAATGCTAACTCGGGCTTTAAACGGCTTCTTTACCAGAGGGTCCTTCAGGCTTGGCGTTTGTTTGGATTTTTTATT
CTTTAATTTTTCAGAATGCAGACAGTCTTTTATTCTGCTCAGCTTGCCTTATGCTCCTGAGTTCAAGAGGCGCCCTGGCTTCTGAGTGACTGAC
TGCAGGCAGCAGCCACTGCTCCTGGCTTCCCTGAGAGCGTTGAGTGTGCTCATCAGTCTACCCAAGAGATAAGATCCATTGCCTTTCCCAAACC
TGTGTTACCCTGGGAGCGTCAGGATGCTGAGAGACAAACCTTCTTACTGGATAATTTCCCACATCACGCAAACCTTACACAACAACAGTCTGAG
TTATTAGACCATTAAACCGAGAAAACCAAATATGAAAGAAAAAGAAGATAACCTTTTTAATGTTGATCAAGAGCACATATCATTCTCTTAAGTG
ACAAATGTCATTTACAGAGAAGATTTTCTTTGAATTTTGCAAGGTTTGAAGCTTTGACCCTTTAGATGTTCTTTCTTATAAATCTCTGAACCTT
GCCTAATCAGGTGGAATAAATCAATATCCTTGCCAACACTGAAAGATTGTTAACGTTGTCGTAGTTGGGGTTGTGAGTCAGTAGAGGACACTTG
CCTGCCATCTGGGTTTGGCTTCAAACTGCATGATCACAGACTCATTATTTAATCAGTTTTTCTTTCTTTCTTTCTCTCTCTCTCTCTCTCTCTC
TCTCTCTCTCTCTCTCTCTCTCTCCCTTCCTTCCTCCCTCTCTCCCCCCTCCCTCCCTCCTTCCCTCCCTTCCTTTCTTCTCTCCCTTCCTTTC
TTTCTTCCATGTTTAGTTCTTGAACACTTCCAGCAACCATGAAAGCTCGGGCTTAGATGCACCTACCTTACCCTCCACTCCTCTCATTGGTCAC
AAACTGACACCATGTCGAGACCAGACTGTGAAAACCCAGAAGGAAAATTCCATGTAAGTCTTAGTTCAATAAGAAAAATGTTTATTTTATTTAT
TTATATTAATTAAAATGTCTGACCCCCCCTTGTATGTATATAATGCATTCACTGTATGTATCCTATGCATTCTCATAATGGATACATCCCAAGT
AAGTTACAACCAACCAGGGAAGAAAGTAGAGAATAATTACCTTCTGTAGTACATTCTGATGTCTTATGTGCATGCACAGGGAGTTTCTGTACCA
TTCCCCACCCCCCACCGGCCAGGTTTTTCTTTCCACTGTTTTTCAATATCCTTACTAAAAACAAACAAAAGAGACTGAAGCATTCAGAATAAAA
TTGAGAATGGTTCATAGTTTATATTTTTTAATCTTATTTTTTCTTTTTGTAACAAATACATTAGGATCTTTTTGCATACTCACAGGAATGAAAA
ATAAAAACAAAGTTTTAGACATAGCGTGTTTTGGCCCCGTTTGAGGGAGCAGGTGAGACTGGTTTACAAATGATCCCAGATATCACGGTGCTAG
TCACCATTATACAGATCCAAGGCCATTACCTATGCCTCAGGCTCATTCTCAAAAGGTGGGAACTACTTCAGAGCTCTTAGTCTCAGGGCACGAT
AGTTTGAAGTATTTGTAGATCTGCAATCTGGTTTAATTCAGATCTACAATCCCTCAATGAATGCATTTGGCTAATCAGCATTGCCCTTCCTTGT
CCGTTGCAGCCTCTTAGTGTCCACGCTGACGGCGTCATACGGGATACATGTCTTTTTATTCTCTGCTTCTTCTCTTCTTTAGCTTTAGAACTCC
AGCTATCAAAAGGTCAATCCTCGAAAGCTCTCCTCGAACTCCCACACCATTCAAACATGCCCTTGCAGCTCAAGAAATTAAATACGGTCCCCTG
AAGATGCTAGTAAGTTCAAGAAAGGTCCTTAGATTTAGTGGGCTTAGGAGTGTAGACAAGTTTCCTCTGATCTGGAAATATCCACTCTTCCCTT
AGCCTCAGACCCCCTCCCATGCAGTGGAGGACCTACAAGATGTGATTAAGCGGGAATCGGATGAATCTGGAATTGTTGCTGAGTTTCAAGAGAG
TGGACCACCGTTACTGAAAAAAATCAAGCAGGAGGTAGGTCGCCCTCGCTGCTTTGTGCACTTTGAACTGTTTGACATCACTGCTAAGGAACAG
TAACACTTGTCATGCAAGACTTTTGTGCAAGCAAGTTGAAAGGCACTGTCATAATGAGGACTTCAACCTTCCATTTGGAAAGTGACTTCCATCA
ACTTCTTTAGGCAGAAAAGATGCTAGAGAGGGGTAAAGGGAAGAGGGGGCAGCGGGCTTATACTCTTCTTTCAAAATTTATTGACAACATAGTA
TTAATCGCACATATGCAAAGAGAAAGACTCCCAACCCAAATCATGACCACATTAATTAGAGCAAGCTTTTTTGTGTGCACAGGCTACTTCTCTG
TAAGTCAAGAGATTAGCATTGGACATGGGGAAAATAAGGGTTTTTGTAGTTCAGGAGTAGAGGGTTTCCAAATGGGGGTTTTGGTAGGCAAAAA
GCCAGGGCTACAGGAGCCAAACATAAGCATCACAAATTGTTCGGAACAACCTCGTAGAACAAAAGCATAGTTAACAGGTGGTCATGACAAGGTG
ATCATAACAACCTTTGAAACACAGACATCATTGCCATGTCCTGGAACAGGCAGTACAGAGCCATTTGTAATTGAGGTTTCAGATGGGGCATAGC
CCAATGCTTGAGAAACAGAGGTTTAATCATAAACAGGAATGGATCTAATTGGTCTTTACTTTAAGATGGCTTTCAAGCCTAAGATGGAGGCAGG
TGAATGGGCAAGAGGATGGATAGACAAGCAGTCAACAGAGAAACAGATTCCAGATTCATTCCTGTTGTCTTTCTAAGGACTAAGAAATAGTTTA
TTATAAGAGTGACCATGCCCAGAGAATGTAGATTCAAGTTACCCTGAGTGGTATGACCCGTTAGAGAACAGGAGTTTGCAAGCACAAGAGAATG
AAATTTTAGCCATCAAGAATCAGTGTTAACTCACTAGGGAGAGGCACTTGCAAGGAGGGTGAACTTCCTTTCATGACGACTTTATTTTTGCAAG
AATAGGTAGCAATTTACACCTGAGAGTAAAGGTCTCCAGAAAGTCTTTCTTCCATACACAGCACTTCCTGTTTGCTTTTAGGTGGAGTCGCCAA
CTGAGAAATCGGGAAACTTCTTCTGCTCAAACCACTGGGCAGAGAACAGCCTGAGCACCCAGCTGTTCTCGCAGGCGTCTCCTGTGGCAGATGC
CCCAGTAAGTATCAGGCGGATCTCGGACTCTTCTGTGATCACGTGGCCACAGCGTCCCGCGTGTGGCATGTGGGACCCCATGCCTTCAGCACTC
ACTCTTAGAGCACCTGCTTCCTAAGTATTCACAGCCACATGATTCTTTTTGCATTTGCATTATTTGTCTTCTAAGTGCTGTGTTTCCTCCTAGT
TAAGCAGTATGTTGTGTGGTTCCATGTGGTATCTAGCATAGAGTCAATCATGGTGTGTACCAAATATTTGGCATGCTAGGAGCCTAGTCAAGGG
GTCCTTATGGAGCCACAGGAGAATGGCCCACAGGCCAGATTCTCACGTTCCACAATTATAATGGCATGGTATTTGCATATAACTCATTGGGTCC
TCCTGTATACTTTACATTAGCTTTAGATTAACTCAACTCTCACATTGTATAAATGTTGTGTTAACAGCACTTCACCGTCTTAGTGTTTTGACCG
TGAATCCGGATGCAAAGTGGAAACTCTGTCACCTTGCTCTGGTTGTCACAGTCTAACAGTCTGTGGATAGCGTCTCCTACATCTTATGGAAAAC
CAGAGCTAGTGAAAATTGTGAATACTTACTCAGTGTGTTAACTGTTAATTATCATTTCAGGGACTTTAGATGAATTAATTCCTGTAGTCCTCAA
AATAACATTGTAACATTTTCACTTTCATTAAAGCAACTGCAAGTTGGGTAACTTAGTGATTTGGTTGCTCAATATTATATACTTAACTTAGTGC
TGCTGCATTAGAACTTTGAAAGTCAGCTCATAAATACCACGCTTTCACCATTTGTAGTTGATGTTGCCGTAGATTTGTAGACGTCAAACACTTC
TGTGCCGATATATACAACATAAGATGACTCATTAGCACATCTCTTAGTTAGAAGAATGGATTGGTAAATTCATTTAGTAGATAGCTAGGACATG
CCACATGCTTAGAATTAAGCGGTGCCCTACAGTGTTTTCACCATTTTGGTGATGTTTGCAGCAGAGAGATGAGAGGCCTGTGTAGAGTTGGCTC
CTTTCAGCTCCCAGAGAAGGTTAATTTCTGGTGGATACTTGCTAATGTGTGGCATTTCACAGTAATCTCTCTAGAGTTAGGTCTGTATTCGCTC
TTGTATTATCCTGCCTCACTTAAATGATTCCTTTCCCTTTAGAATATTCTTACAAGCTCTGTTTTAATGACACCTGTATCAGAAGATGAAGACA
ATGTCCTCAAAGCCTTTACCGTACCTAAGAACAGGCCCCTGGTGGGTCCCTTGCAGGTAATGACTGTTCTAACATTTGGTGTCTTAAAATTTTT
TAACTGAAAGTCCGTTACTTTGCGTTGTTTTTAATGTTTTATACATAAAGGAATGATACTTTAAGACAATGTCTTGATGTCTAGAAGTGAGCCT
ATGGTGTTTTGTTACTAATCATGTGAGACACTGGAAATTCTTGTTCAGTCGGGGGCCCCCAAACATGATGAAGTGAAACATTTTTTTAACTGTA
ACTGAGACTGAGATCATGTGTTGCCCTAGGCTGTGACAGTATCTCCTTCTGCTTTTAAACAATCTTTAAAATATGTATCTGTGTAGTTTTTTTT
CAGAATGTAATTATGTATGCTTTATCATTAAGCAACCAAGGCCTCAGAGACTAAATTTCTAATAAAATGGGAAATTAGCTAGAAAATAATTGCA
ATGCCTTTGACTCTCAGAACAAAAGGGCAGCCCTCAGTGTTGGTGGACCATCTGCCGTCAAGTTTTAATATCAAAAGAGATTTCCCTTGCAACT
CTGAGGTAGATGTGTATTGATGAAATACAACAAATTCCAAATTAATCCTGAGCCAAGTGTAAATAAGAATGCAAATTTGTTCCCTCAAATTAGT
TGAAGCAGTTCCCAACATTAAATAGCGTAGCACCTGGCCAGCTGCACTTCTCTTCCCTCTGTTCCCCCATTGTTACTCAGGGGGGAAACTCTAC
CAAGCAGAGAATAGCCTGGCTAGAAGTAAAAAGTACTATTTGATGCTGTATGCTTAAAGGCAATGTAAAAGATAGTAGCCTTTGAGAGCTGGCC
TCTCTGTTTATAGGGCCGTCTTGCCACTGATATTCTGTCTACCCCAAGTTGTGAGAATCTTAAAGTCTAACCTTGTATTATTTGCTATCATTTA
AAATGTCTTTGTTAATTCTTTGAGGATTTCATGCATTGTGTTTTGATGACATTCATCTCCAAGTCCTTTCCTTAACTCTTCGGACACCCCTGGT
CTCCTACTTCTTGTCTTCTTCGTCGTTGTTTTGTTCAATAACCTACTGACCTAATTTGTGCTACCCATAATACTCGAAGCAACTCACTGTAGTA
AGCTCAGCCTGCCAGGAGTCTCACACTCTTAAAGAAAACTGACTCACCCTCCCCCAGAAGCCACCAACTGTCCATAGGTCTGCAGTTGAGGGTA
AGGGTTTATAAACCCCTCCCACATCATCGTTGATTGGTCTTGTGTAGGCTCTGTGAGTTTGTGAATGCCGTGGTCCTGTCATGCCCAGAAGACA
CTGATTTTCTTCATACTTCCGCAGTCTCTGACCCTTAGAACCTTCTATCCCTCTTCTAAGTGTTGGAGAGAGCTGTGATATGGATATTACATGG
TGGCTGAGGACTTCTCAGTCACGTGTTCTCTATGTTTAATAACCTATTGACTACAGCAGTGAGTTTCTGCTTTAGCCATCATCCAGTAAACCGA
GAAGAGTATCTGATGAGGTCTAAGAGCTTCACTAATATACAGGTGAAGAGATAGGAATTTAGAAGATAGATTAGTATTGTTCCCATTTAGCAAA
GTAACAGTAGTAGATTTACATATGGGGCCTATGAACTGCCTAGCCATGGGTTCTTGGCCAAATTTGCAGGCATATGCTAACTCCTATGAAGCAA
GTCTTAAGTTTAAGAAGAAAACGGTTGGTTGCCTCTCTGACGTTTGTGCCACCGTTGCTGCCATGGGCATATCTTGTCACGGGGGTCATTATTG
AGGTTCACAGTGTTCACAACTGGGTAAAACTGATTGGAGCTCTCTGCCCTGTCAGCAGCCTTGCATGATGTCTTCTGGTATTGTGAAAACTAGC
AAGGATGGAGGACTCTTCCTGGTCAGTATGAATCCAGAGAGGCAAACATACCATGTAGATTTTATTGCTGAATCTTTGTATTTGTGTGTTTAAT
TTGGATGGTCGGTATAATCGAGGAATCTGGAAAGGGTTCATTGGTGGGGAAAGGGAAGAAAAGATCTTAAGGAGGGGAATGGTTAAACAAAGAG
GAGAAAATGGGGAATAATGGAAGGGAGGCTTAAGTGTGGGGGATGAGAGGATAGACTAGAGGATGGGTTAGGAGACAGACAACTAACACTGAGA
ATATTTGGAAAAGCCACTTGGAATCCCACTATTTTATAAACTGTATAATAATGTATCCTTTAATTCATCCTACAAAGGGGAGTATGCTTATTTT
AGAAGCTATAGAATAAAAAGCCCAGTGCTAGGTTCAGGGTACCTCTCCACTAGTTGTTGGTCAGAGGTGTCCTAGGAACCCTGAAACAATACAG
GCTACTGCCATTGCTCATGGTATTAAGACTCTGTTGCTGAAGACACCACATGCTTCAGTCACAAGACATGGAGAAGTCAACTTGGTATTTGCTG
TCACCTTTTAGGACAACTGGATGAACTGTAGGCATGGTTATTTCTTAACTCAGTTGGATTGGGTCCCTGTAGCTTTCCTCAGGTACATGAACTG
CTCTGATGAAAGGTGGGTGGGCAGGAGTACGGAAGAGGAAGACCAATACACCAAGTCAGCTCTTAGGGCAGTCAGCCTTCTCTTGCCCTATGTA
GAGAGAACTCAGAATACCTCAAAGATATGGAAGGTAAAGAAGAAAGCACACTTCACCCAGGTCTAGTGGGATACAGGAAGGGGATTCAAAGGAT
GTCAGAGCAAAGGTAGCTATTTAATACAGTACTGATCCTTGGTGGAGAGGAAACACCTTGCTTTTTAATTGGCCCTTTGGTTTCCAAAGCATAG
AATTTGACTAATGCTGTTTGTATGTTAAGCTACATAAGTTTCATGCATATTTTATAACCTAGCCCGATATTCAGCCTATGTTTATTGAAGTATA
AAGTGTAGCCTGACATGAGGTTGCATGTCTTTAATCTCAGCATTTTGGAAGCAGAGGCAGGCAGATTTCTGGGTTTGAGGCCAGCCTGGTGTAC
ATAGAGTTCTATATACCAGCACAGGTACATAGTGAGAACATGTCTTTAAAAATAAGACGAGAAAGGAAGGAAGAGAGGTAAAGAAGAAAGAAAG
AACATTCAGAACGTCAGAGTAAGCGTGTGAGTGTATTTGGTGGCAGTAATCATTTCATATATTGAAAACTCATGACTGTGCACCTAAAATGGAA
TCCTATCAGTTTCCAGCTTTACCATGCAGTGGTTTAGGCCTAACTATTCAGAGCTGAACATGGGCTAGCACTATTTTGCTATATTCTTCCTTAC
CTGTGAGGGTATAGTTTTTTTCCCCTCCCCACAGTGGTACCTGAATTGTGGCTGATATCAAACATTACATATATGATGCTTTTACATATCTATC
ATAAAGTCTAATCTATAGACTAGGCAGTAATGAGATACAGAAGTATGATAATAAACTGTAATATAAAGCATCCTAATCCTATGAGTTGTTTATT
TCTGCATCTTCTGACTGCATTTGCCCCAGGTAACTGCACTACAAAATGCTGAGACTGGTGCACAGGGAGATCCACTGTTTTTTCCTGTACTGGG
AGCTTTGTTTGTTGAAGCATAATGCCATTTCTAGTGTTTCCACCAATACTTCAATTGGATCTGATTAGGCAGATCTTCCATATGTGGTATTGGT
GAGCATACTGTTCATTTATTAAATAGTCTAACTTGGATACTTTCTTTCCCTTTTACTATGAAATAAATATCTGACAAAAGCAAATTAAGGAAGG
AAGGGCTTGTGTTGGCTTGTACGTCGTTGGAGGGTGCCAGCCATCATGGCGGCAGGAGCTTGAGGCAGGCAGCGGTCACATCTCCCCCAGGCAG
GAAGCACAGAGCAATCGACACTACTGTTCAGTTTGCTTCCCCCTTTTTATGCAATCCAGGACCCCAGTGCAGAGAACAGCACTGCGGTGTTTAG
AGTATGTAGTCCCAGTTCAATTAACTAGTCACGATAATCCCTTACACATACTCCCAAAGGCTTGCCCCTGGAAGTGACTCTATTTTGTCAAGTT
GCCGGTCACTATCAGCCTTCTCACTGAATAAAGAACATTTCATAGAATCTAAAACTTTTCTCATTTGTACTTTTAAGATAAATTAAATTTTCAA
TGGATCTTTAGCCAAACCATTTGGATATTCTTGACACTGCATAGTTTTTCGTTATTTTGGTTTTATTTTCTTGAAATGAGCTCTTACTATATTG
TGTAGCCCAAGCTATCCCCAAATTTGGGACAATCCTTCTGCCTTGGCCTTCTGAGCTTTGGGATTGCAAATTGAAATTCACTGTGCCTAGCTTT
TACCTAAAACAACAAAGCACTATTCCCCAAATATTTTACAAGTTGACTGTTTTATAAAATACAAAATATTTGCCACTTTTCTAGTGTAATAAAT
TGCAAATGTAATTAAAACCAGTTTTAATANNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNCAGTTTCCTTTACAGGGTAAGAAACATGCTTTATGCTAATAAACA
TTTCTACTGCCAGATTCAAACCCATGCTCTTGCTCTTAGCCGCCTGAGCTTCATTTTCTTTTACGTTATCATTCCTTTTTAGTCCTGTTTGAAA
CATGAGTGCAAGCGCTGTGTTTATATGCGTTCTTTACTCATGCACTCTCCTCTGGATTGTTTTGTAACCACAGAATGACAGCCTTAAATTTTAA
TTTCCTGACTTGGTTTGTCAAAGCCTTACTCTTAAAAAAAACAACAACAACAACAAAAAAACCAATGAGTTTGAAAACATTTCTTCAGTCTTTA
ATACTTCAGCCCATCCTCAGAAGTTCCAGAATTTTACAGGACTGTTAGCAGAAATTGCGGCTCTCTTCCCTCCTCTGTTCTGTGTAATTCAGCC
GGTAATTGTCTGCATTTCACTCGTCTGTAAGTGTTCATACTTTTCCAACAACACCCTTTCCTTGTGACTGATGTTGCTCCGAAGTGACATGAGT
TCTCACATGGGCATCAGAGCCTGGGCTCTGCTCTGGGCTCCTTCTCATCCTATTTGGAGGACTTGATTAAATGGAGCAAATGAAATGAATTAAG
TTACTCTTGTCTACAGTTAAGGACCGTGATGTGTCCTTCCCTGGTCTCTTAAGATTGCTGTAAAGATCATATGGATCGGATGATTCCAAGATGG
CAGCACATAAGCCAGCCTGCTCCCATGATATTTATTATCAGAGAAAAACTGCATGCAGCCTCTGTCCTGTCAAAGAAATGCAGCTGAGCTGGCA
GAGTGCTAGCACAGCATGCATGAAGCCCTGGGTTTAATCCAGCACCAGGTGTAGTAATGTAAATCTATAATCCCAGTCCTCTGGAAGATCAGGA
GTTCACCTGTAACTACATAGTAACTTTAAACATAGTCTTGGTTATATAATGAAAAAATCTAAATATAGCTTAAAAATCTTAATACAGTAGTTAA
AGAGCAAGTAAGTAGGTCTCCCTATTGCCAGGACTTCTTGTATTATGTTAATAACGTCTACAATTCCTATTCCACCTTGCAGAACCAGGTCAGT
GGGTCACTAAAAGAAATAGCTGAGCAGATCAGGTAGTCAGGGCTCCTACTACAGCAGAGATGAAGCAGGTGACGGGCGGCCTGGGTTTCTTTTC
TTTCCTGCTGTTGTGTGGGTAGCAGGAAGATGGCCGCAGATACAGAGTGCATTCTGAGTGTCTGTCTGTCTGTCTACTGTGCTGCCGGGTTTAA
CTCCAGGGTGTCTTCGCAATTGTTGCTATTCTCTCTGGTCACTATTTTTCCACCCATGTCACTCTCAGAAGTGTTTCAGTGACCAGCATTCAAT
TAGTGATATAGATTAAGTAAGCTTTCTGTGTGTATCTCTGGGATGGAATTTCTCTGATGAGCTGAGTCGGTCTGCCCTGCCCAAGTTGTTCCAA
GACAGTAACTCTTCATACACTTTGTTTGATGATATTTAAAACAAACGTGGAGATAGAAGTTGCCTTGGGTAGGTCAGAAAAGGTAAATTGCTTC
AGAGAGACTTGACAGTTGTCTGACTACTGATAATCAAGCACTAGGGGATGTCCGAGGAAGGAGCTGTTCTTCCTAAAACTTGGGTACCCCAAGC
TATAGAATGTAAGTTATTGAGGTGTCTATAAACATGTTAGAATGAACACTTCTAGAGACATCTGGATAAAAGCCCATATCTGCGAAAGATCCCT
GTGCTCTACCCATTGTTAATTCAGCCTGGACACAGAGGCACTAATGGGTAGGGGGCATCCTGCAGTAATGAGTAAGGGGCATCCTACACTAGTG
GGTAGGGGGCATCCTGCACTAATGAGTAGTGGGGCATCCTATACTAATGGGTAGGGGGGCATCCTGTACTAATGGGTAAGGGGCATCCTGCACT
AGTGGGTAGGGGGCATCCTGCACTAATGGGTAGGGGGCATCCTGCACTAATGGGTAGAGGGCATCCCGCCACCACCTACATTGTCCATCCCTTC
AGTTAGGAACCCTGAAGTTGTAAGTATTTCATTGTGCTGCCACAGGTATTCACTAGTGAGGAAGTTTGCTACCCATTGATCTCACTGTAGCAAC
CTCTTGATACAATGAAGATTTTCTGTGGTATCCTTTATGAATGCCGTTTTACGCAGATGTCCCTGTGGGAAGGAATTCTGACTTTCAGAATCTC
ATTAAGGATGAGATGAGTAGTATTTTTGTTTGGATGAATTAAATAGAATCGGAGAATTCTTACTCTGGATTCCTTAGACACTATCAATCCATAT
ATATTAGTTTTTTTAAAAGTAATATAAATATCAATAAGATAGTAATTATATGTTATAATAATAAAATATTAAATAAATAATAAAATATATATTA
GATTCATCTCGCTGTCTTATTACATCTTCACATTGTAAAATAGAATTTCCCAGGCGCCTTGACCTCAGCAGATACTGTGTGGCTTTATGAATGT
AATAATACAGTTATTCAGCATGAGGGGAGCCCACGGGAAATGTTGCAATAATCTAGTTCTGTCAGAAAACCACCTGTGGGCGTGGTCTCGGCTC
CTTTCATTTATTGGGCTTTTGCAAAGGGTCTGGATGTTTTTTTTTCAAAGTGCTTTAACAGGATATCCCAGTTAAAGTTTTGACAGACTGTTTA
TAGCTCACGTCTGCCCAGCGGCAATGAAAGATGCATTACAGGATAAGCAAGGTGACTCTCCTGGCCGCCACAGGAGGATGTAGGGTAGGATTCT
ATTAACATTTAAATGAATTCAGACAGGCTGACATCTACAGAACACTGGGTTTAGAAGCAGCAAGAATTCACATGCAGAAGCTGCTGCGCCTAAC
TGCTCTCTTAAAGTAAACAGAAATGCAGGAGTTGAAAACAGAAACACACAGAAATTGAGGTGACAGCATTCAAAAATAGCCCTTAACAGAAAGA
CACCTAATCCTGGTTTGTCACAGCTATCATGTTCTTGAGAAGTTGCTGTGGGGTCCCCAGTTCAAGATCCTAAGGAAAAAGGAAGCTGACCTTG
ACAGTCTTTTTTTCTCTTCTCTTCTTCTCTTCTCTTCTCTTCTCTTCTNNNNNNNNNNNNNNNNNNNNCCTCCCTCCCTCCCTCTCTCCCTCCC
TCTTTCTGTGTATGTGTTTGGGAGAAGCACACAGATGGTATGTTGCAGACAGAGGACAACCTTGGATAGTGTTCCTCAGAGTCTGTCCATCCCA
CCCTACCATCTCTTTTGAGACACTCCCTGAAACTCATCAAATACCTAAGTAGGCTAGGCTGGCTGGCTAGTGAGCCCTAGGGATCCACCTGCCC
CTGCCTTCTTAGTGCTAGGATTCCAAGTGCACACTACCGTGCCAATCCCACCCCCCTTTTAAAAAACACAGTTTCTGGAAATCCAGCCCAGGTT
CTTCCTCACTGAAGAGTAAACCCTTTACTCCCCGGGCCATCCCCCTCGCTTGAACCATAGTGCCCGCCTACCTGACCTTGCTCTCCATTCTCGC
CATTGGTATGTGTTCTGTTCAGCTTATTACATTCTCTGTGATGTACCTACTGTGTGCCCTCCTAGCTCCCAAAGCCGCTTAGCTCATGCGAGCT
GCCTTTCTGCCCACAGTGATTCCACACACGCTAGCTTACTGCACCCTATCGTGTGCACACCAAGAACTGCGTGTTCTGACCTCTCCCTAGCCCT
AGGCTCTGAGGCTCTGACCTAGCGCTAGGCTCCGGCTTAGCACACGTGCTGAGGAGTCTGGGTATTTTCATTATTGTGAATTTGATCTTTAGAC
ATTCTCTTTTGTAGGAGAACCTGATTATTTGAAAAGTCCCCACAAAGTACTCATGAGAACTATATAGTACATTCTTTATATATTGATTCTATCT
TAAATATCTAGTCCTGGAGGAAAGACAAAATATCACTCCAGGGATGATCAACCTGGAGATTTATCAAAGTCACAATTCTTTTCAAATTGATAAG
TTGATATGATTCTCTGAACAGGCATGGACAGTTAAGAGTCATCTAACATACCATACAGCAAAGGACGGCTCAGTGTGTGGCCATCAGCAGGAGC
AAACATAACTTATATTGTGGTGGAAGCTTAAGAAACTTAATTTGCATACAGGAATTTTATTCTGTATTAAATCAGGCCAAGTAGAAAATTTCTT
TTTTTTAGTAGGAAAAGCTCTTAGAAAAAAATAGAAATAAGCCTTTTTTGTTTCAATATGTTTTTTCCTTCATTCTTCCTCCTCCATTCTTTTG
AAGATACATGTTTTTGGTAATACACATACATATATATATGTGTGTGTGTGTGTGTGTGTGTATGTATATATATATATACATATATGTGTATATA
TATGGTGTAATTTTAAATCTACATGCACAGTATCATTAGGTTAAGTTATACAGTAGCCTCCTCACTTAGTCAGTGTTTCTAAGCACTACATATG
CTTCTCTGTAGAGGATACACCATTTGATTTGTTGTCTCGTTTTTTGTTTGGTTTGGTTTGGTTTTTTTGAGACAGGGTTTCTCTGTGCAGCCCT
GGCTGTCCTGGAACTCACTCTGAAGACCAGGCTGGCCTCGAACTCAGAAATCCGCCTGCCTCTGCCTCCCGAGTGCTGGGATTAAAGGCGTGCA
CCACTACTGCCCGGCTCTGATTTGTTGTCTCTTAACTGCTCATTATTATATGTCATATTTTGCTGCAAGGATTACAGGCCCTAACTCAGTAATT
AAATTGTATTAGTTTAGGTGATATAGTGTGTCTGTTTGATTTCTGTGCTGCACTGGAAAACCTTCGCTCCTGCAAGTTAGCAGGCAGTAACACT
CAAGCAGAGGCGCTGGGCGGACTGAAACCCTGCGCAGAGCCATGTGTGTTCGGTCTGTGCATTTGGATCCAGGGAAATACTCATTTTACAGCAC
TTCTCAGTCTTCCTAATCTGCAACCGTTTAATACTACTACTGTTTTGAATTGTAAAGTATGAATCTGTGTTTTCCCATAGTCTTAGGTGACTCC
CTCAACCCTCCCCCACACCCTCAAAGGAGTTGCAATCCACAAGTTGAGAACAGCTGGTTTAGAACCATTTCAAAGGATGTTGTAACTAAGCTTA
TTGTTAGGAAAAATATCAAATGTGACTTTGTGTTCAGAAGTCAACCTGGGATGGCTGCCATCTTGGTTGCAGCCTGTCTGTGACATGCTGTCCC
TTGGAATGCTGTAGGACGATTCAGTGGCTATGGAGTATGTCAAGCTGCACCTGATTGTTTCGAGCACACTTGTGCACTTGGCTGTGACTGAGAG
GCAGCCAGGCAAGCTAAGAGGCTGTGACTCAGGGAAGTCAGAACACAGCGCGCTGCAAACTGAAAGTCACTGGAACTTTAGTGTAGTCACTGAG
GGGGGTGGCGGGGAGATCAGGACCTCAGCACCATGTCCAGCAGTGTTCCTTCAAGGTTGCCATAGTTACTCACCAACAAGCATGCCTCTTCTCA
CTCCACCCCCACCCCCACCCCCATGGTTCTGTCTGTACTGGAAGGTCCTGGTGATAAACCCCTGAATTTCCTATGCACATCCCTGCTACGTTCC
TCATAAGAGGTCTTAGAGGTATGGGCTGCTGAATCCCACCTCATTCAGATTACTGTCCCCTCGTTCCTTAGTGATCTAGTCTTGAGTGTGTTGC
TCATGTGCTGTCTTCTATGTTAACTTCCTGCTGTGTGACCTATGTGGGGTGCTACAGAGGTGGCCACACCATGGGTGTTCTGTGTGAGAGTTTA
AGTGTGACAGGATCACAGCAGTGCAGTGTAAGGGCCGTTCCATGATGCCTTGCAACCACTACATCCTTTGTTTCTCCATAGTCTTTCTCACCAA
CACTGTTCAAACCGAAACTCCCCACCCTCACTATCCTTTCCCTCCGGCCGTTCTTAAACAGCGTTCTCAGCGTCCTGTGTCCTGTTCTCTTGCA
TGTGGCACTGCAAACCGCCACGCGCCGGGGTTTTTTTTTTGCAGGGTATAAAAGCACATGTGTGTTTTATCTCTCAAAAGACGCTTGAGGTTCT
GGAGAGCAAAAGCTTCAATGTTGGAGACAAAGCTATGTAGCCCATGTTGGCCTTCAATTCACTAAGTCTCAACATTCCCTTCCTAGTGTTCTTG
CTTTTTGTTCTTTAAAAAATAATTTATTTATCTACTTTATGTGCATTGGTGTTTTGCTTGCATGTATTTTCTGTGTGAAGGTGTCAGATCTCCT
GGAACTGGAGTTACAGACAGTTTTGAGTTGCCATGTGGGTGCTGGGAATTGAACATGGGTCCTCTGGAAGAGCAGCCAATCAGCGCTCTTAAGC
ACTGAGCCATCTCTCCAGTCCTACTTCTCTACTTTAGAACCACTGTTGTATTTGTGCTATGAACGCTGTAAACCATCCTGAGAATTAAGTGGGA
CATAAACTGTACATATTCCAGTTTGAGTAGACATGGAGAAACAGCAGGAACCAGGAGATACAGTGGGCAGAACACCGTTCAAGCTATTGTGTGT
ACACCATAGCCTCACTTGGGCAAGTTCTCTCTAAACCTCACCCCTTCATTTACCTGTCCAGCTACGGTGTGTCTGAACCTTTCCTCGTGGTGTG
TCTGCACTATGTACATGTGACTATGTGCATAAGACCCAGTACTGTCCTGGGTATCTGTATGTTGCTCTTCATTACTTTTATCTTGTGATTTCCA
ATGTATCCCTTAAAACTACGCTAACGTGGGGCTGGAGACATAGCTCAGTCCGCAGACTTCTTCTCTGCCATACATTAAACCCTGACCCAGCACT
TGGGGAGTTGAGGCAGCACTTGGGGAGTTGAGGCAGGGGAGTCGAAGTCAGCTCAACTACACAGTGAGTTCAAGACCAGTCTGAGATACATAAG
GCTCTGTCTAAAAGGAGAGGAAAAGGTTAAGCTAATCTAGGAGTTTGGGTTGTAGTTTAATGGTAGAGCATTTGCCTAGAAAGTGTGTGAGGCT
CTGGGTTTAACCTTCAACGCCGCAAAAAGAAGCTGTGCTGATAGACATACTAATAAAGATAACTTTGAAATGTACATGTGTGCAAATAGATGTA
TGTGTTTTAGATATTTGTATGAATTGTGTGGGTTTTATCAGGTAGTGGTACATTACAGAAATTACGCTGCTTGGCTCAAGGGCTCGTCAAGTCC
ATGGAGTGAGTAAAGGCCTAGGATCTATCTGCCTCAGGTATCTGTAGAGTGATGCCCAGATTCTGTCTCCAACCTTGGAAAACTGCCTGCGAGG
CAGAAGGCATTCCAAGAACAAGATGTTACATCTTACGAAAGTTCTGAGAGACAAAGGCTTGCTAGCATGGACTGCTTCCCGCCTGGCTGCCCTG
CTAGGGTGGGGAAGCCTGGGCCTCCTGGCCTGACCCTAATGTCTCCTTCTCCTCCCTCATTAGCCATGCAGTGGTGCCTGGGAGCCAGCATCCT
GTGGGAAGACAGAGGACCAGATGACGGCCTCCGGTCCGGCTCGGAAATACGTGAACGCGTTCTCAGCTCGAACTCTGGTCATGTGAGACATTTC
CAGAAAAGCATTATGGTTTTCAGAACACTTAAAAGTTGACTTTCGACACATGGCTCCTCAGCGTGGAGCGCTCCATGGCTGAGAGAAGAGCCTG
ATTTTGTTGTGGTACAACAGTTGAGAGCAGCACCAAGTGCATTTTTAGTTGCTTGAGATCTCACTTGATTTCACACAACTAAAAAGGATTTTTT
TTTTAAAAATAATAATAATGAATAACAGTCTTACCTAAATTATTAGGTAATGAATTGTGACCATTTGTTAATATCATAATCAGATTTTTTAAAA
AAAATAAAATGATTTATTTGTATTTTAGAGGATACAACAGATCAGTATTTTTGACTGTGGTGAATTTAAAAAAAAAATTTACACAAAGAAATAT
CCCAGTATTCCATGTATCTCAGTCACTAAACATACACAGAGAGATTTTTAAAAACCAGGAGAAGCATTATTTTGAATGTTAGCTAAATCCCAAG
TAATACTTAATGCAACCCTCTAGGAGCTCATTTGTGGCTAATAATCTTGGAAATATCTTTATTATACTAAACCATTTCATGAGGAGAATTTTGT
TGTCAGCTTGCTTGAAAAGTTATTACTGTATGAAATAGTTTTATTGAAAAAATTATATTTTTATTCAGTAATTTAATTTTGTAAATGCCAAATG
GAGAAATGTGTTCGCTGCTATGGTTTTAGCCTGTAGTCATGCTGCTAGCTAGTGTCAGGGGGCAATAGAGCTTAGATGGAAAAAAGAGAAAGAG
ACTCGGTGTTAGATAACGGACTATGCACTAGTATTCCAGACTTTTTTATTTTTATATATATGTACCTTTTCCTTTTGTAATTGGAAAACTTATT
TGGGAGAATTTTGCATTTGTTGTACATTTTTGTTTTTTAGGATTTTTTTTTTTGTTGTTATTGTCGGTTTATAAAAGCATTGCACTTCTTTTTC
TTTTTTTGGGAGATTTGTGTTGTTTATGTCATATGTTTTGTTTTGAGTTCAGCCTGAATGTTCATCCGTTTGGGCGTTTTTCTGACTTGGAAGA
ACATTCTCTGTAGGTTTCTAAGTGTACAGAGCCGGAACTGCCTCGTGGTTCCTGGGCTTCAGGGAAGACAAATATGGAAGTCAACAGCCAGTTT
CTGCCTTGAGAGCATTTGCAAGAATGCTGGCCTTGAATTCTGAAATGACAGTGTATCTACTGCCTTGTAGCAAAATAAAGCTATCCTCTTATTT
TACATACTTCCTGATGTCTCTCGATTCTGTGATTTGGGGTGGTGGTTACATATTCGAGGAGATGAGACAAACTCAAAATAATCTTCACTTTAAA
AAAAAAATGTGCTTGCCATCTAACTTTTTACATGGAGAGTCATCGCCACTTCAGGGAACGAAAAGACAAACCAAGGTAAGTCCACAGAACCGTC
ACAACGACCGTTGGAGGGCTCTTCCAGCCTGTGTCAAACAAGGGCACTGAAGCTCTTTAATTGAAACAATGCACTCTAGAAGTAAATGCTGGTA
TCTGAGTTAGGGAGATGGCTCAGTCTGGAATGTTCTTCTGTCAAGAATATAACCTGAGCCGGGCGTGGTGGCACACGCCTTTAATCCCAGCACT
CGGGAGGCAGAGGCAGGTGGATTTCTGAGTTCGAGGCCAGCCTGGTCTACAGAGTGAGTTCCAGGACAGCCAGGGTTACACAGAGAAACCCTGT
CTCGAAAAAACAAACAACAAAAAAGAATATAAACCTGAGTCCCCATCTCCAGAAGCCCCACAAAGCCAGACAAGGCATCCATGTCTATAGTCCC
ACCAGACAAGGCGTCCACGTCTATAGTCCCAGTATTCCTGTGGCAAAAAAACGGGAGTTGAAGATGAAGATTCTCGGATGCTGGCATGCACCCA
GCAAACCCACACAAGAAATGGCAGAGAGCTAAGAGATGCTGTCTCAAGACAAGGTTTGAGGGGAGAACTGATAGCCAAGGTTGTTCTCCATAGA
GGACATGTGGCATGCATATGCCCCGCCTCCACCATGCACACAGAGGCTCGAGAATTTAATCGCATATAGGTAGGCTGTGAGAACTGAAAGGTCA
TGCTCATGGAAATTAGGAGACTCTGAGAGATGCGATGAGCCCTGGTGATGCAAAGGCTTTCTCTTTCTGCTTCCCAGCCACTCTGATGCAAACT
GCTCCACTTTCCCATCGTAATGGGCTGACATCTCTGAATCTGTCTTTCCACAAGCTGCTTCTATATTTTATCACAGTGAAAAAAAGTAACAAAC
ACAGGTGTTCCAATGTGGTCTATTCAGTGTTCAGTATCCCTTGCACAAATACTGATTGATTCTCACAACTCAAATCCAAACTCCAATTTTGAGT
TTTCATAAGCATAACATAGTTGGTGTTCATCAAGCCAAGGATATGATAGATCTAATGACTAATCTTCAGAGCTATTTTATAATCCTTGATCCTG
GTGAGCAATACATTTTCTGAGTGTTGAGTAGTATAACTGATGAGTAGCTTTTGTCAGAAAATGGAGCCTTGATGACCAGTTAGCTATCTCAGTG
AGTCAAAGTATTAGAGATGATGAATGTGACTAGCAACGAGGAGCCTAAGTGTGTGGTTCAGTGGCAGGGCAAGAGCTGAGCATGTGAATCCCTC
TTATAGAGGCTTCGACCCTCTATAAACTCTTACCTATATGTAAATCATAGTTATATGGCAGGCAACTGGGAATGGCTGTTTTACTACCCAGATT
TTTCTAGTGACTTTCAGGAAGGCAGTGGTCATAGGTTTATAACACAATGCTTTAAAGTATATTGTTCTCTAGTTCTGGGTGCTAGAAAGTCCAA
GTTCAGTAGGTGGATTTAAAGTCTGGCGAGAGCATACTTCCTGTTTCGTAGAAGATACGATTTTGTTGTGTTTTCTGTTTTCACATGACAGAAG
GCCCTGGTTTATTTTGTTGATTGGGTGGGTAGGTAAAGTGGGTGGGTGGTTTGGTGGGTGGGTGGGTGGTTAATTGGGTGGGTGGGTGAATGGT
TGTTTGGTTGGTTGTTTGGGTGGGTGGGTGAATAGCTGGTTAATTGGTTGGTTGGGTGGGCGGATGGTGGTTATTTGGTTGGGTGGGTGGTTGG
GTGGGTGGTTATTTGGTTAGGTGGTTGGGTGGTTGATTGGTTGGTTTGGTTATTTGAAACAGGGTCACCTGTAACCCAGGCTGGCCTTGAAGTT
GTTATGTAGCTTTGAATGGTCTTGAATCCCTGAACTTCTTACTTCCAATTTCCAGGCGCTGGGATTATAGGCATGGGCCAGTATACCCAACTCT
TGCTAGTTTACTTTGGTTGTTGGTTTTGTTTCATGTTTTAGTGACCCTACTCTCTGTGACACATGTACAAATGCATGGATGTACTATATTTACT
ACCTAAATGAGAAGCTATTCCAATGTCACTATAAAGTGGCATGTGAGGAACAGGAGAGACACTGAGAGGACCAATACTGTCCACTGACCTCATA
TAGGACTGGTAGATGCGAGTGTTGTCANNNNNNNNNNNNNNNNNNNNTTCTGCATCACCAGGGTCCTGTGTTAGGGCCCCCTGCATTGCCTCAT
CTGATCTGAAGTCTGACAAAATTTGGTTGTAATTCAGACTTTACCATCGTTGTTGACTTCACACAAGTTTGATGTCATCTCTCTAAGTTCATTT
TACTTATATTGGATATCAAATATAGGTATTATGAAGATGTTGTGAAACAGGAAATCCATTCATGGGTCTCTCTTAAGATTCTGTGGCACCTGTC
CGTAAGATGCTTTGCTAGTTCATCTGTGCAAACAAGAACTGTGTGTGCCATTCGGTGATGAGGAATGAAAGATTTCTGAGGCTCATGTCCCTGC
TTGCAAGTTACATGTACTCAAATATAAATATTCCTTCCTAATATTCTCAGTTATTCTACAGTAGATAAATTCTTCTTATGTAACATATATGCAA
ATGACAGCACAGCTTCCCTTCAACCTCTTGGTTTCTAGTGCAGTGTATCTTACACACACTCGTTCTCATCCATTGCCGTTCTCATCCATTGCCG
GGGGGTGATATCAGCAGGGATGCAGGGTGGCTAAGTGCTTACTGCATCTACTGTCAAGGAGGGAGGTGACCTGGTTCCCAGCAGTGAGAAGTGA
GAGGCGAGGGGCAAGATAGGAGGCTGTGAACTGAGGTTTGGACTTTGTCCTTTGCGATATGGGGGGTTATTAAAGGCTTTTAGGTAGAGAAATA
AAATGATTAATTTATATCTGATGTATAGAAATCCTGTTCAAACTCTTTATTCACATATAGTATAAAATTAATCCTCAGATTAGTAGGGAGAGAT
GATCAGAGTCCAAATTCCATGCCAGTTAACTTATTTTTCATAGCATGTTTCCTGCTGCCTAATTTCAGAAGCATGCATTTTAATCCTAGAAAAT
CTAACTCGGGGGGGAAAAAAAAACCAACTCAACTTTGATCTGTGCTTCATGAAACATCATTTCAAGTGTTATAATCCATAAAGTAGAAATTGAC
TCAGGAAGAAACGATTGCCCAGAACACTTTTCTCCTTTAGAAGTGGGACTTTTCCAACCATTACTTTCTCAGACCTTCTCTGCTAAACCTCCCA
CTGAATCATTATGTGGTCTAATCCTTGGTTCTTTATGGATATTTTGATGGATTTTTGTATTCACTGATTAAGGAATCTTTCCAGCATTTGTTCC
TGAAGAAATTTCAATGTAGTGTTATATTGTAGTCTACCCTTCACACTAGAACACCAATGATCAGTGGGTCTTGGGTTTTATTGGACACAGAGGC
TAAGAACACTGGTAGATTGAAGTTAAGTCAGGAATGCGTCCTGTGTAAACTCTGCATCCCATGTTTGACAGCCACCAAGTGACTCCAACACAGG
AGGACTGAGGGAATTGAACTACAAAGACCCGGTTTTGTGAACAAATTTGCAACCTGAGCTTTCTATCTGACCATTCCGCAAGCCTTACCCGTTT
CTATTGAAAATGTCCATTGGTCTGCAACAGAACCTTAGAAAACATAACCCACAGCTACAGGGTTTTCTTAACTCTAAGGATTTAATTTGTTACT
AGTGTTGGTAATTAGGTCTTTTGTCTGGTGTTTACAATTGCAGGAAGCATAATTTCATTCACAATACACTTGGGTCTCTCTTATCATATGGCTC
TGTTGTCCGCGTCCCATCAAAAGGGAATATACATCGATGAATTCAACATTAAAGCAAAGCAAAGCTGTTGTTCAGAGAGATTCTTATTTCGAAA
CCAGGCTGCAATGAACTTGCTGATCTCCTTTGTTTGCCTAAACCTCTGAGCTTTAGGTTTCCGTTCAAATGGAGGAAGTATTTAATCCTGTATA
TAGTTACAAAGCATACAGAATTTCTCAGATCAGAGATAAACAAAACTCAAAGAATGCTTCAGTAGTCCAAAGCCATTGGAACACACAGTGTGGG
CTGCGAGATTTTTAGGTCAAGAACACCCTCCAAGATGAACCTACTATGCTTGGAGAATATGTAAGGACAGAGTCACGGGTTGTTTTCATTTGAC
TGATCTCTTTAAAAACATAAGTGTTTTCCCACTTTCTAATAGTACTAAATATATCTGACCATGGATGGAGCAACTCATCCAGCTAGTAGGGTTA
GAACCTACCACTCATTTTTTTCCAAAAATTCTTTTTTTATTGAGCACTTCCAATGCAAGGCCGAGTTCAGCTGAGTATATACAGCTCACAAACA
TAATAAGTACACACAAGTATAGTGTGTACAACATTCACAATTATAGGCAGGATCATTTTCTTGGATACCCTCGAACATATGAAGGAAAAAAATA
GGCTTATACTTTGGTAACTATGTTTCTTTAAGTCATACCTGTCACTGTTTAGGTAACCAAGAACCAGAGGTTAGATGGACCAGAGACCTACAGT
AAGAAAGCATAAAAGATAGTGATAAAACGACCCCTAATGATATTCTGCTATAGTCATAGATCAGTGGCTCTGTTCAGCCATCAGAGAAGCTTCC
TCCCACAGCAGATGGAAAGAAATACAGAGACCCACAGCCAGACATTACACACAGAAAGACCTTAGAACACTCAGCTCCAAGTGGGATGTCTCCA
TCCATCAAATCTCTCCCCTCAGAGCTCAGGGACCCCCCCCACACACACACACACACACACAGAAGAGGAAGGAGGGTAAGAGACAGAGGAGGTG
GGCCACCAGGAAAACAAGGACCTTTAAGTTTACATGAACAAAGCACATATGAACTCACAGAGACTTAGCAGCAAGCACAGTGCCTATACACGCC
TGCACCACGTCCTCTGTGTGTATATAACGGCTTTCAGTTTTGTACTTTTATGGGACTCCTGTCTGTGTTAAAAATGGGTCGCTGATTCTTGTGC
CTTCCCTTGGGGCTCTTTTCCTTCTGTTATTTTATCTTGTCCAACATCAATGCGATGGTGTTCTTTCTTATTGTGTTTTATTTTGTCATATTAT
TATTATCATCATCACTTAGAAGCCTATTCTTTTCTCCTGAGAGACAGGAGGGGAGTGGATGCAGACGGGAGCCGAGGGACAGAGGGCCTGAGCA
GGATGGGGGATGGGAAATGATCATCACTCTATTCTGTATGAGAGAAGAATCTACTTTCAATTAAAGTGAGGAAAATCTAAGATGTCTCTTTTAA
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNCTGGACTACA
CACAAGTCTGTGGTGCACTTTTTTGATTAGTGGTTAATGTGGGAGGGCTGGGCCACCGTGGGCAGTGCCCTACCTGAGCTGTGGTCCTGGGTTG
AATAAGTAATTGGGCTCAGCCAGACACAATGAATAAGCCAGTGAGCAGTGTCTCTCCATAGCCATTGCTTTACTTCCTGCTTTCAGGCTCCTAT
TCTATATCCCTGTCCTTGCCTTCCTGGGTTCCCGCTCCGATGAACTACAGTCTTTAAGGGTCCTACTTCCGTTTGGACCTACTCCTGTCTTGTC
CATTCCTCCTCCATCTCCCTGGGCTTGAACTGCCTGCTCTTGCTATCCCTATACAGTGCACTTCAACTTTTATCCTGGACACCTGACTCACTGA
CAGAGTCACAGGGCTGGTTTCTGTCATCCTACATCTGGTAAGTCCTTGATTCCTCTCCTTGGTGGTATTTGATCTTTATCACTACTGAGAAGGA
AGCCCCAGAAGTTTCTTTTTTCTTCTTCTTAATAAAAAGAGGGCGGGGCTCAAACCATGACTCAGAGTTGGAACATTAATTAATAAAGGAACCT
TTTCTAGGCCAGCAGACAGGAGTTGCATTCTTGCCAATCCTTTCTAAGAGCGGATGGGCAATGCCTGCATCCATCCTGCCTAAGTCATCTTGAA
ACTGAGGGTAGCTGATGCTTTTATTGGCATCCTTCCTTTGAATTTGACTTTTCAGCTCCTCTGTGGTTTTGTCTGCAGACCCTGGTGGTGAGGT
GAAGGGGCAACGCTTTGCTCACACTGCAGCGTTCTTCACGAGCATATTCGTCCCTAGACTACTGAGCAGCCTGAGCAGAGATGAATACAGGACA
GACTGATGAATGGTTGTGAATGGTAGATGATGAGTCGACATAGCAATTACAGTCCAATGTTAATTGTCAACTAGACAGGGAGTATGCGTGTATC
TGCTTTGACTTGACATTTTTTTCTAAATGTTTACAATACTCAAAATGTAGAGTTTCAGGGTTGGATGACTACAAATCTAAAGGCTTCGACATTG
ACACCATAAAATCCCCTCTGACTTGAACGCCCTGAAGCTCATCATGCTTCTAAGTTGTTTTGTTTGTTGGTTGGCTGTTGATCATTTTTTCATC
TTTAGGTATATTCTTGTGATTGTCCAACATGGAAGCTTTAAAAAATGGGTATTTTTTCTTTTCAAAAAGAGAGCTGGGAGTAGTGGCACACACC
TTTAATCCCAGCACTTGGGAGGCAGAGGCAGGCGAATTTCTGAGTTCGAGGCCAGCCTTGTCTACAGAGTGAGTTCCAGGACAGCCAGTGCTAT
ACAGAGAAACCCTGTCTCAAAAACAAAACAAAGAAAAGAAAAGAAAAGAAAAGAAAAGAAAAGAAAAGAAAAGAAAAGAAAAGAAAAGAAAAGA
AAAGAAAAGGAAAGAAAAGAAAGAGAGCTTTAGGCTGGCCTGGAACTCACTACGTAGCCCTGGCTGGCCTTGAACCTATAGCCATCTTCCAGCA
TCATCCTCCTGAGTATGTTTGATTACAGATGTGAGCTACCAGACTCAATTCAGTTTGTTAAAATAATAGCCAATTGTGGTCTTTTGTGAACCCG
TAGTCCGGGATTTCTTCAGGTGTCTGTATCTTGCTCAGTTTTTGGTCTTGGGCTTGGTGAGGGTTAGCACAGCTCCCGGGGGCAGGAAGAGAAG
TACAGAGCAGTTAGTTCATCATCACAAGATCAGCTAAGCATCTGAGAGAGGGCCCTGGGTTCCATCTCCTATTGGCAAGCAATAGACTCACATT
GCAGAGAGTATAGAAGGGCATCAACCAGTTTTGGACTCTGTATTAGTCAGGGTTCTCTAGAGTCACAAGACTTATGAAATGTCTCTATATATTA
AAGGAAATTGTTGGACTGACTTACAGTCTGCAGTCCAACTAATCCAATGATGGACAACTGTGAATAGGAAGCCAAAGAATCTAGCTCAGCCCCA
CAAGACTAGTTGTTTCAGCTGGTCTTCTAGAGAAGCAGGTTCCAACAGATGTGCTGGCAAGTAAGAGCAAGCAGGTGAAGAATGGACCCTTCCT
TCTTCTATTGTCCTTACATAGGCCTCCAGCAGAAGTATGGCCAAGAGTAAAGGTGTGTACTACCATGCCTGGACCTAAGTTGTTCTTTACTTGG
AACTTGCTCTGTCCCAGGCTGGCCTTGAACTCAGAGATCTGCTTGCCTCTGTCTCCTGGGAATAAAGGTGTGTACTGTCTTGCCTGGTCCTAAG
CTTTTCATGGCCACTATGCCTCAAGATCTGGATCCCAGGTGTGCCCTCCATTTCTGGATTGTAGTTCATTCCAGATATAGTCAAGTTGACAACT
GGGAATAGCTATATTACAGACTCTATCAATATAATCACCCCCTTAGCAGTTCATCTTTTAAAATATCATTAAAGGATGTGGAGAAATGGCTCAG
CTGATATGGGGCATTAGTTATGGAGGAAGTGTCTGTGCTGGGCATGGATGCATTACAATTCGGAGATGTGATTAGCAAATGACCCTGACAAATA
CGGTTAGCAAATGTAATTTCTCCTTCCCTCCCCTCCTTCCTTTGGATGAATAAATAGGTGTTTGCATTTATAAATATATAAGTCTCCCTTCTTA
CCTACACTTTGCTTTCTGAGCTTAAGTTTACCTGAAATCAAGCACAGGTCAAAAATATTAAGAAGAAAATTCTAGGAATAAACAATTCATGCAT
TTGAAACTGCTTTTATTATAGTATAGTATAACAATTACTGTATGCTGTTGTTATTATCACTGCATCTAATTTATCACTTAAACCTTACCTAGGT
TTATAGGTACTGCAAACACACACACACACACACACACACACACACACACACACACACACACACACACACACACACGTTGTGTATGTTGGTTAGG
GCCAAGACCGCCAGGCAGGAGAACCCGAACTCCAGCTGCTCGGCCTGATTCTGATGGAAACCCTGACTTCCGGCCAGCTCACTGCCTTTCGGGT
GCTGTAGGATCCCTGCCCTCCCCCAGCATCAGCTTCCGCAGAAGACAAGTGTGCTCGGGGCTCCTCTTCACACCCTTGAGCCCACACTCCCACG
GGCG
MOUSE SEQUENCE - mRNA
GGGAGTGTCCAAACCTCTTTGTTTGATGGCATCTGTTTACAGAGTTACACTTTAATATCAACCTGTTTCCTCCTCCTCCTTCTCCTCCTTCTCC
TCCTCCTCCTCGGTGACCTCCTTCTCCTCCCCTTTCTCCGGAGAAACTTGCCCCCGGCGGTGCGGAGCGCCGTGCGCAGCCGGGGGAGGACGCA
GGCAAGGCGGAGGGCAGCGGGAGGCGGCAACCGGTGCGGTCCCCGGGGCTCTTGGCGGAGCCCGGCCCGCCTCGCCATGGCCCGGAGACCCCGA
CACAGCATCTACAGTAGCGATGAAGATGATGAAGACATTGAGATGTGTGACCATGACTACGATGGGCTGCTGCCCAAATCTGGAAAGCGTCACT
TGGGGAAAACTAGGTGGACAAGGGAAGAGGATGAGAAGCTGAAGAAGCTGGTGGAACAGAACGGAACAGACGACTGGAAAGTCATTGCCAATTA
TCTGCCCAACCGGACAGATGTGCAGTGCCAACACCGGTGGCAGAAAGTGCTGAACCCTGAACTCATCAAAGGTCCCTGGACCAAAGAAGAAGAT
CAGAGAGTCATAGAGCTTGTCCAGAAATATGGTCCGAAGCGTTGGTCTGTTATTGCCAAGCACTTAAAAGGGAGAATTGGAAAGCAGTGTCGGG
AGAGGTGGCACAACCATTTGAATCCAGAAGTTAAGAAAACCTCCTGGACAGAAGAGGAGGACAGAATCATTTACCAGGCACACAAGCGTCTGGG
GAACAGATGGGCAGAGATCGCAAAGCTGCTGCCCGGACGGACTGATAATGCTATCAAGAACCACTGGAATTCCACCATGCGTCGCAAGGTGGAA
CAGGAAGGCTACCTGCAGGAGCCTTCCAAAGCCAGCCAGACGCCAGTGGCCACGAGCTTCCAGAAGAACAATCATTTGATGGGGTTTGGGCATG
CCTCACCTCCATCTCAGCTCTCTCCAAGTGGCCAGTCCTCCGTCAACAGCGAATATCCCTATTACCACATCGCCGAAGCACAAAACATCTCCAG
TCACGTTCCCTATCCTGTCGCATTGCATGCTAATATAGTCAACGTCCCTCAGCCGGCTGCGGCAGCCATCCAGAGACACTATAACGACGAAGAC
CCTGAGAAGGAAAAGCGAATAAAGGAGCTGGAGTTGCTCCTGATGTCAACAGAGAACGAGCTGAAGGGACAGCAGGCATTACCAACACAGAACC
ACACTTGCAGCTACCCCGGGTGGCACAGCACCTCCATTGTGGACCAGACCAGACCTCATGGGGATAGTGCACCTGTTTCCTGTTTGGGAGAACA
CCATGCCACCCCATCTCTGCCTGCAGATCCCGGCTCCCTACCTGAAGAAAGTGCCTCACCAGCAAGGTGCATGATCGTCCACCAGGGCACCATT
CTGGACAATGTTAAGAACCTCTTAGAATTTGCAGAAACACTCCAGTTTATAGATTCTTTCTTGAACACTTCCAGCAACCATGAAAGCTCGGGCT
TAGATGCACCTACCTTACCCTCCACTCCTCTCATTGGTCACAAACTGACACCATGTCGAGACCAGACTGTGAAAACCCAGAAGGAAAATTCCAT
CTTTAGAACTCCAGCTATCAAAAGGTCAATCCTCGAAAGCTCTCCTCGAACTCCCACACCATTCAAACATGCCCTTGCAGCTCAAGAAATTAAA
TACGGTCCCCTGAAGATGCTACCTCAGACCCCCTCCCATGCAGTGGAGGACCTACAAGATGTGATTAAGCAGGAATCGGATGAATCTGGAATTG
TGGCTGAGTTTCAAGAGAGTGGACCACCGTTACTGAAAAAAATCAAGCAGGAGGTGGAGTCGCCAACTGAGAAATCGGGAAACTTCTTCTGCTC
AAACCACTGGGCAGAGAACAGCCTGAGCACCCAGCTGTTCTCGCAGGCGTCTCCTGTGGCAGATGCCCCAAATATTCTTACAAGCTCTGTTTTA
ATGACACCTGTATCAGAAGATGAAGACAATCTCCTCAAAGCCTTTACCGTACCTAAGAACAGGCCCCTGGTGGGTCCCTTGCAGCCATGCAGTG
GTGCCTGGGAGCCAGCATCCTGTGGGAAGACAGAGGACCAGATGACGGCCTCCGGTCCGGCTCGGAAATACGTGAACGCGTTCTCAGCTCGAAC
TCTGGTCATGTGAGACATTTCCAGAAAAGCATTATGGTTTTCAGAACACTTAAAAGTTGACTTTCGACACATGGCTCCTCAGCGTGGAGCGCTC
CATGGCTGAGAGAAGAGCCTGATTTTGTTGTGGTACAACAGTTGAGAGCAGCACCAAGTGCATTTTTAGTTGCTTGAGATCTCACTTGATTTCA
CACAACTAAAAAGGATTTTTTTTTTTTAAAATAATAATAATGAATAACAGTCTTACCTAAATTATTAGGTAATGAATTGTGACCATTTGTTAAT
ATCATAATCAGATTTTTTAAAAAAAATAAAATGATTTATTTGTATTTTAGAGGATACAACAGATCAGTATTTTTGACTGTGGTGAATTTAAAAA
AAAAATTTACACAAAGAAATATCCCAGTATTCCATGTATCTCAGTCACTAAACATACACAGAGAGATTTTTAAAAACCAGGAGAAGCATTATTT
TGAATGTTAGCTAAATCCCAAGTAATACTTAATGCAACCCTCTAGGAGCTCATTTGTGGCTAATAATCTTGGAAATATCTTTATTATACTAAAC
CATTTCATGAGGAGAATTTTGTTGTCAGCTTGCTTGAAAAGTTATTACTGTATGAAATAGTTTTATTGAAAAAATTATATTTTTATTCAGTAAT
TTAATTTTGTAAATGCCAAATGGAGAAATGTGTTCGCTGCTATGGTTTTAGCCTGTAGTCATGCTGCTAGCTTGTGTCAGGGGGCAATAGAGCT
TAGATGGAAAAAAGAGAAAGAGACTCGGTGTTAGATAACGGACTATGCACTAGTATTCCAGACTTTTTTATTTTTATATATATGTACCTTTTCC
TTTTGTAATTGGAAAACTTATTTGGGAGAATTTTGCATTTGTTGTACATTTTTGTTTTTTAGGATTTTTTTTTTTTTGTTGTTATTGTCGATTT
ATAAAAGCATTGCACTTCTTTTTCTTTTTTTGGGAGATTTGTGTTGTTTATGTCATATGTTTTGTTTTGAGTTCAGCCTGAATGTTCATCCGTT
TGGGCGTTTTTCTGACTTGGAAGAACATTCTCTGTAGGTTTCTAAGTGTACAGAGCCGGAACTGCCTCGTGGTTCCTGGGCTTCAGGGAAGACA
AATATGGAAGTCAACAGCCAGTTTCTGCCTTGAGAGCATTTGCAAGAATGCTGGCCTTGAATTCTGAAATGACAGTGTATCTACTGCCTTGTAG
CAAAATAAAGCTATCCTCTTATTTTACAT
MOUSE SEQUENCE - CODING
encoding71kdmybprotein,completecdsATGGCCCGGAGACCCCGACACAGCATCTACAGTAGCGATGAAGATGATGAAGACATTGAG
ATGTGTGACCATGACTACGATGGGCTGCTGCCCAAATCTGGAAAGCGTCACTTGGGGAAAACTAGGTGGACAAGGGAAGAGGATGAGAAGCTGA
AGAAGCTGGTGGAACAGAACGGAACAGACGACTGGAAAGTCATTGCCAATTATCTGCCCAACCGGACAGATGTGCAGTGCCAACACCGGTGGCA
GAAAGTGCTGAACCCTGAACTCATCAAAGGTCCCTGGACCAAAGAAGAAGATCAGAGAGTCATAGAGCTTGTCCAGAAATATGGTCCGAAGCGT
TGGTCTGTTATTGCCAAGCACTTAAAAGGGAGAATTGGAAAGCAGTGTCGGGAGAGGTGGCACAACCATTTGAATCCAGAAGTTAAGAAAACCT
CCTGGACAGAAGAGGAGGACAGAATCATTTACCAGGCACACAAGCGTCTGGGGAACAGATGGGCAGAGATCGCAAAGCTGCTGCCCGGACGGAC
TGATAATGCTATCAAGAACCACTGGAATTCCACCATGCGTCGCAAGGTGGAACAGGAAGGCTACCTGCAGGAGCCTTCCAAAGCCAGCCAGACG
CCAGTGGCCACGAGCTTCCAGAAGAACAATCATTTGATGGGGTTTGGGCATGCCTCACCTCCATCTCAGCTCTCTCCAAGTGGCCAGTCCTCCG
TCAACAGCGAATATCCCTATTACCACATCGCCGAAGCACAAAACATCTCCAGTCACGTTCCCTATCCTGTCGCATTGCATGCTAATATAGTCAA
CGTCCCTCAGCCGGCTGCGGCAGCCATCCAGAGACACTATAACGACGAAGACCCTGAGAAGGAAAAGCGAATAAAGGAGCTGGAGTTGCTCCTG
ATGTCAACAGAGAACGAGCTGAAGGGACAGCAGGCATTACCAACACAGAACCACACTTGCAGCTACCCCGGGTGGCACAGCACCTCCATTGTGG
ACCAGACCAGACCTCATGGGGATAGTGCACCTGTTTCCTGTTTGGGAGAACACCATGCCACCCCATCTCTGCCTGCAGATCCCGGCTCCCTACC
TGAAGAAAGTGCCTCACCAGCAAGGTGCATGATCGTCCACCAGGGCACCATTCTGGACAATGTTAAGAACCTCTTAGAATTTGCAGAAACACTC
CAGTTTATAGATTCTTTCTTGAACACTTCCAGCAACCATGAAAGCTCGGGCTTAGATGCACCTACCTTACCCTCCACTCCTCTCATTGGTCACA
AACTGACACCATGTCGAGACCAGACTGTGAAAACCCAGAAGGAAAATTCCATCTTTAGAACTCCAGCTATCAAAAGGTCAATCCTCGAAAGCTC
TCCTCGAACTCCCACACCATTCAAACATGCCCTTGCAGCTCAAGAAATTAAATACGGTCCCCTGAAGATGCTACCTCAGACCCCCTCCCATGCA
GTGGAGGACCTACAAGATGTGATTAAGCAGGAATCGGATGAATCTGGAATTGTGGCTGAGTTTCAAGAGAGTGGACCACCGTTACTGAAAAAAA
TCAAGCAGGAGGTGGAGTCGCCAACTGAGAAATCGGGAAACTTCTTCTGCTCAAACCACTGGGCAGAGAACAGCCTGAGCACCCAGCTGTTCTC
GCAGGCGTCTCCTGTGGCAGATGCCCCAAATATTCTTACAAGCTCTGTTTTAATGACACCTGTATCAGAAGATGAAGACAATGTCCTCAAAGCC
TTTACCGTACCTAAGAACAGGCCCCTGGTGGGTCCCTTGCAGCCATGCAGTGGTGCCTGGGAGCCAGCATCCTGTGGGAAGACAGAGGACCAGA
TGACGGCCTCCGGTCCGGCTCGGAAATACGTGAACGCGTTCTCAGCTCGAACTCTGGTCATGTGA
HUMAN SEQUENCE - GENOMIC
TTGAGAAAAACATGTAACAGTGTGAAGGTAAAAGGTATCTTCTTTTAAAAAGTATTTTGCATAGATATTGTTCTTTAAATGTTCCATGAGGATA
TCCTTAACTACCAGTTATTTAGAAGAAAGAAAGAAGCAGCAAAAGAGTTTTCAAAGAAAAAAAAGTATATGCAATCTAAAAGCCAGCTAAGGTG
CTATGCACACCCTCTAAGTCCCAGCTGCTTGGGAGGCTGAGGCAGGAGGATCACTTGAGCCCAGGAATTCAAAACCAGCTTGGGCAACATAGCA
ACATCTCATCTCAAAAAAAAAGAGAAAAAGTTATCAAAACTCCATGTAACCTAGATTAAAACATAATTAGAAATTTTATACATATATCAAAATG
TCATATTGTTCCCCATAAATATATACACTTATAATTTATCAATTATTGATAAAGAAATAAGAGCTTTTATATCACGATTTTGGTGTACCTGACA
TTTTTCAAACAAATTCCGCCTTTTGGGGCCAAAGTGCTCTCTGTCAAGCTCCATACTAAAGGATCAAATGAGCCTCCCCAAGCTCCCAGCAGAA
CTAGCACTTTGGAACTTTGTCAAAATTTTAAGACAAAGGATTATAAACACTTTGATATAGTTTCCTTAGAGGCTGAATGTTTTGCTTAACACAA
ATATTTAAACCAAATTAAGATAACTCAAGAACTGATATGTTTCATAAACTGATTGTTTTTATTTTTCTCAGAACCGTTACCTTAACTGAAGAGT
AAATTGGAATAAGCAATTTCTATAGATGTCAAAACTTTGTAAAATTGATTTTTTACATCATCCACAAACTTTGTATTTCTAGCAACAAATGTAA
AAGAAAGAGTAGAGTTATGGTACACTTCATCCTAACAGGGTTCACATTTAAAATCATGTAGTCCACTTCTGTGAAGGGCCCAAGAAAAATAACT
CCCTTAAATCCGTTCCTTTCTTCTTGTTCTGTTCGAGTTAAAAACTTTATTTTCTTAGACTTTGGTTAAAAAAAAAAAAAAAAAAAAAACAAGG
AAAGACATAGTGGTGATTAAAAAACATGCCATTTCTTACCAAATGGTTTTCACAAAGCCTAAAAAAACCTACAGTTGCTGGAATTAAGCACTCA
TTCACTTATTGATTTTATGACTAATTTTAAAATGGATGGCCCATTGAGGTAAATTCAATATTATATAAAGATGTAGTAAAGTCAACAATCAGAT
AAATATACAATGATAAAGTAGGAGAGACAGAAATAAGATTCAGAAAAATAATATGTGAAACATCATGCTTTAAAAGAAAAACAGTTCCAAAGTC
ATATTGCCAAACAGACAGAAATCTATCCAAATCATAGAAATAAAGCACTACAAGTAAGCTTAAAATTATGATTTTGCTATCATTTGTAAGGTTA
TCAACCTTTATCTGAGGACAGAATTGAATCCCTTTGAAGGACTCACAATACAGGAAGAATTACAAGAACATAAAGGCATAGGAGTTAAAAGTTG
AAGGCTGCCAGAGAGTAGCGGAAAGAACTTTGGATTTGGAACAAAAAAACCTTCATTTAAACAGTCCCAGCTCTGGTAAATGTTATCTGTGTGA
CCTTGTGCAAATTACTTCACCTGTTTCAATTTCAGTTACTTGACCTGGAATTGAGGAAAATAATACCTGCGGGGGAGACATAGGCAGAAAGGCA
TGAACTGCTGTGAGGATTAAATGATATAATATATGGCACAGCCTGTTGTAATCTCTAAAGCCAAATATAACGTGCTAATAAGTATATTAAATAA
AAAATAAATAAAACAGCTCCAACTAGTTCTCATCAGTTGTCATCAGCACAACAAAGATTTATGTACGTACTTATGTTCAAGACCCCGTTCTAGG
AACTGAGTAGGTAGAAAGGTAAAAGATACTGTCTCTCCTCTCTGACCTATAATGAAGGCTCCAGATGAAATAAGACACATTTATAAATAGATAG
ATAGATGATGCTTAGATAGATAGATAGGTAATAGATGACAGATAGATAGATAGATAGATAGATAGATAGATACATAGATAGATAGATACATAGA
TAGATACATAGATAGATAGATGATAGCAAAGAAAGGTAACATAGGCCATACACTCAACAAGAGTCAGGGAAGGTAGGTATTAACACAACTCACA
GGTGGGAGAAATGACTGTGGATTAGCAAGGTCCAAGAAGGCTTCAGAAGGAAGATTTGGACTTCAGCAGGGCCTGAAGAATGGGTAGCACTTAC
ATAAGCAGAGAGGAGAAAGGAAGGCATTGCAGGCAGGAGGCATGCTTGTAACCAAAGGCCTGAGATTGCCAATGGTTTTTTGGGGGAGGAAACA
GAGTGAACAGAGTATGATAAGAGATAAGGCTGAAAGGTAGGTTGTGTTCTAAGGCAAAATGGCATGCAGTCAGTGTGCCCGTCAAGCCCTGCAG
GTTGTCTATAGCCAGGCCCACTCTGATTGGCCAATACATGAGCCACTTCAGGTATTAAATACTATCACCCCTGGGGGTAAGGTTGTCTGGGCAC
AGAGGCCAGAGCAAGCTCAGTATAGGGAATGCCAAGTTAAGGAGTTCAGATCTCTATCTTGTCAACATTAGGGAGCCAGTTAAGGGTATTTAAA
CAGATGACTAACATGCTAAAAGTGAGTATAATGAGATTATTGAGCAGTGGGATGCAGAATGCATGGGGGAAAAGTGGCCACAGGGTGGAAAATT
AATTAGGGGGCTGTTATGGTAGTTCAGGGTGAAGTAATAATAGCCTAGAAGAAGGTGGTGACAATAGAAAGAGAAAACAGGCCCTGAATGTGCA
GGAATCATCTAGTAAAAGTTCCACTTCTTTCTTTCTGCAGGCCCAAGCCTTGCACAGTGCCCAACACTAAGTAAACACATGTGTTTATCAACTA
TAAGGAACTATGGAAACATTGTCAGGGATACAAAGGTAAATTGAGAGACCTGAGTATTTAAGAAAGAAACAGAATATTAAAGACTGGAAACATT
TTTAAAGATCTCTACTGCAGCCTCCATGGATTGAAGGTTTGAAATATGAGGCTCAGAGAAACTGTGTGCTGCTCATACATTTGATGAACCTGCC
TTAAACACCCTGTTATTTGTTATTCCAATAAGCCCCTAAATGTTATTAATCTTTTGTTGTTGTTGTTTCTATTAGCATTTCTACTAATAAAAGC
TTACTGTTTTGTTGACTAACTTTGAAAGCTGCTATTTTACATTTATAACATATGTGGGAAAGTATAGAGAAGGAGGGGAATTGACTAAGAACAG
TTGTGAGAAGAGATAAGAGAGAAATTACATCTGTAGGCCGGGTGCAGTGTCTCACGCCTGTAATCACAACACTTTGGGAGGCCCAGGCAGGCGG
ATCACGAGGTCAGGAGTTCAAGACCAGCCTGGCCAACACAGTGAAACCCCGTCTGTACTAAAAATACAAAAATTAACTGGGCGTGGTGGCAGGC
GCATGTAATCCCAGCTACTCGGGAGGCTGAGGCAGGAGAATTGCTTGAACCAGGAAGTCAGAGGTTGCAGTGAGCCGAGATCGTGCCATTGCAC
TCCAGCCCAGGTGACAGAGCTAGACTCCGTCAAAAAAAAAAAAAAAGAAAGAAAGAAAGAAAGAAAGAAATTACATCTGTAAACTGGTCTTGAA
GAATTTCCCCGAAAGCTTTAAAAAGATAGGACTGTCACTTGGAGAAAAGTTAACGATTCCATAAAACAGATATTCTAAAGCAGAAAGAGGTAAG
ACCTTCATCCTCTACAGCAATGTTTTTCAAACTGCGATTCACTACCTATAAACAGAGCACGAAAACAACTTTGGGATCCTGACCAGAATGAAGA
GAAGAGAAGAGAAGAGGTCTCAGATTGCATGCTTGGTAGTAAGGGGTAAGTATTGTTTTGTGAAACTTTTAAAAATTATTATTTAATAGGGACA
AGGTCTCACTATGTTGCCCAGGCTGGTCTCAAACTCCTGGTCTCAAGCAATCCTCCCACCTCGGCATACCAAAAAAGTGCTGGGATTACAGGTG
TGAGCCACCACACATGGCCAGAACTTTTCTTAGCTCTGTGTGTGTGTGTGTGTGCGTGTGCGTTGAGATAGGATGTAAGATGTGCTTCTTATCG
TGTCACATATAAAAATATTTGAGGCCGGGTGCAGTGGCTCACGCCTGTAATCCCAACACTTTGGGAGGCTGAGGCGGGTGGACCACCTGAGGTC
AGGAGTTCGAGACCAGCCTGGCCAACATTGTGAAACCCCGTCTCTACTAAAAATACAAAATATTAGCTGGGCGTGGTGGAAGGTGCCTGTAATC
CCAGCTACTCGGGAGGCTGTGGCAGGAGAATCGCTTGAACTCGGGAAGCGGAGGTTGTCGTGAGCCAAGATCGCACCATTGCACTCCAGCCTGA
GCAATAAGAGCGAGACTCTGTCTCAAAACATATATGTATATACGTATGTGTGTGTGTATTTGAAAGCCACCTTTTCCTCAAATAAATCAAAGCC
TTTCAAAATCAGTTGAAAAAATACAGAATTTTATTAAAAGAAGAAAACATTTGTGTGTGTGTGTATATATATATATAACTTTCTTTATTCCCCA
ACAGGTCTCTCCACCCAGCCTCTACCTCTCAGTCTCAGCCCTGTCTTTTAACCTCTTAAATATTATCCAAAGACTTTCCCTTGGTCAGATCTCT
ACATTAGCAACTCTATGAAATAGCACAGTTGTTGCCTTCATGTAAATAAACTCTTAAATAAAGCATTTATTTGATGTGCTACTGGTGAATGTTT
AACAATCAGTTTTCCAATAAGAAAAAGCCCTTATTTGTAGCACTTGCCATTTCCTGTGGTGTAAATACTCCAACTAGAGCCAATTTCAAGTTAC
TAACATTACATCACTACATATGAAATTGGAAAGAGATGCACAACAGCACATATTACATTGTGTGATAGACATAAATAACCTCAAGTATGTAGAT
AATAGGAAAATGTCATAAAATAGGAAGTGATGAGTTTTGAATATTTATTGTCTTTTAAAATAAAATATATTTAACTGTAAGTTTATATAATTTA
CTTTTTAATAATGGCTGTGTCTAACAATTAACTCACAAAATTTAGCTCCCGTGTATTCCTGACCCAGCTTGACTTCGCCAGCAGGCTCTGATCC
CACACTCTCTACCACCTGCTGAACAACTCTACCCGGATGTCTTCCATAAGAAACCACAAATTTAACATACCGAAATCTAAACATGCTTTCCTCT
TTGAATCTCCCAGTGCATACTTCTCTTCGCCTCTTGACTTCCCTATTTCTATTAATGGTGCCTCTAATATCCTTAGCAACCCAATCAGTTGCTA
AGTCTTGAAGGCTCTGCCTATGTTCTTCTGATCTGCCTCCTCCTTTTCATTCCACTTACGGTTACTGTCCTACCCCAGACAATTACCTAGCCTC
TTCCCCTTGTCTCTTCTCACTCCAATCTATCTGACAATACTGATAGATTAATCTTCCACACACACAGCTCTGATAATATAGCTTTGCTATTCAA
CGCACATACATGAATCCGGCCTTCAAAGCTCTCCTTGCTTCAGGCAATCTGGCTAGCTCCTTCTTTCCTGTGCTGGGAACATGCCTTGTGGATT
CTGATTGTGTGATTTTGCTCTCACTTTTGACTTTCTCTGGACTGTCCTTCCCTCTTTATGGAAAGACTCATAACTTTTATACAAGACAGCTCAA
ACAACAATAAAAATATGTGGGTTGGTTGTCATAGGAATATGTAAGTTGATGAGAATCTGGAGAGCGGAAACAAACAAGAAGTGCCAAATCCAGG
AATCATTTTAAAATAAAAATTTATTTGGTCACTATCTGTTATGGAGGTTCTATGAAACATACTACACTGTTATGGAGGTTCTATGAAACATGCT
ACACTGAGGTAGCACTTAGTCCACTGTCATTACTAAAAGATTTTCTGGGGGAGTCAACATGAGATTTATCTCAAAACTATGGATTTCAATTATA
CATGCCACAGCTTTTCTTCATTAACTGAAATAATTAAGAATTGTCTGCATTTTAACCATTTACTGTAACTATTTCCCTCTTAATGCCCTGAGCC
AAACTGGGTTGAGTGGGTGAGGAAAGGTGGGTGGTAGAGACATGGAGCCCCTAATCTGGTCTGTTTTCACCATTGATGAGCCTGATTCATCTTT
GGGATGTGGATCACGAGGATTAAAGATCCACCACGTGCCCAGGATTTCCCAGCAATTAGAAGCAAAGAAACTAGAATTTGTATCTTTTATATAT
ATTTTGTCCTTCAGCCCTTCTCCAGTGCTTAGAGTAAAATAAAATCCCTTAAGTGCTTGCTTTTTTCAGCAGGAGAATCCTAATGATGAGCAGC
ATGTGATCAAGCGATTCCTTTTCCTTCAAGCTGAGGTTGAAGTTCAGTAGCGAAGGAAGAAGGAACAGGCAGCTACAGCCCTCTGGTCAGCAGC
ACCAACTTTCTCACGGAGTTAAATTAGTTTGGGTTATGGAGAGCCTGAATCTTTAGCCCAGGAGAGGTGAAAGTGAATGAGGGAGAGGAAGGCG
GTGAGAGACAAATGGCAAAGGTAAAAGTGGGCTGGGTGCAGTGGCTCACAACTGTAATCCCAGCACTTTGGGAGGCTGAGGCAGGAGGATCAAG
CGCTTGAGCCTAGGAGTTTGAGACCAGCTTGGGCAATATGGTGAGACCTCTTCTCAACAAAATATTTAAAAATTAACTGGGCATGGAGGTGCAT
GCCTGTAGTTCTAGCTACTTGGGAGGCTGAGATGGAAAAATCACTTAAGCCTGGGAGGTGGAGACTACAATGAACTGTGATGGTGCCACTGGAC
TCCAGCCTGAGCAATAGAGGGAGACCCTGTCTCAAAAATATACATATTTTTTTATTTTTAAAAAAGGTAAAAGTGTATCAAAGCAGTCTGCCTT
CACTCTGATACTGAGATTATCAACCAGTGCTCTGCAGACCTTCCATGAAAACTACTGTTAAAGATGCAGATTGTTAGGCCTCATCTCAAACCTA
CTGAATCATGGTCTCTAGGGGGACAGCCTGGGACTGTGCATCTTTCATGGGTACACCCAGCAATTCTGATACACTCGAAAGCAAGTTTGTCTAA
CCTGTGACCCGCAGGCTGCATATAGCCAAGGACAGCTTTGAATGCAGCCCAACGCAAATTTGTGAACTTTCTTAAAACATTATGAGTTTTTTTG
TGGTATTTCTTTAAGCTCATCAGCTATCGTTAGTGTTAGTATATTTCATGTGTGGCCCTTCTTCCAATGTGGCCCAGGGAAGCCAAAAGATTGG
ACACTTGGGCTCTAAAGATTGAGGATCACTGCTCTAGTGTTGGCCAACTACTCTAGTGTTGGCCTGCAGACTCAATACAGCCTTTGAGCTAAAA
ACGGTTTTTACATTTTTAAAGGGTTGTAAAAAAAGACAAAAAGAAAGACAGACAAGGAAAGACACAGAAGAATATACAACATAGCCTACAAAGC
ATTTACTATCTGGTCTTTTACAAAAGAGTTTTCTGACCCCTTTCTAGTGCACTGCAAATGAAAGGCTGTACCCAAATTTAGCTCCAGTGCAGCC
CTGCCACAGCTGAAATAACTGATGTAGCAAGCAGATCTTAAAAAAAAAAGTGATGAAATGTGTAAAGTTGTCATCTAAAAATACTTTTGTAATA
ATTATAAATGGAGAGAGAAAATGTATAAATACAAACATTCTGTAATGAGTTTAAAAATATAGTTTCATCCATACAAGAATTTTGATAACTCCAT
TTGATTCTGTTTTTCCATAAGCTTATCAGATAGCAATATATCCAACATTAGCCTAATAAAATTACTGCATGTGAATTCTTTATTGGACTATGCA
TATTCCCAAACATATTATTGTCATACAACTGAGGGAAAAATGTAAAACTATATCTGGGAAATGAAAAATTATAAATTCTCAAAATGATACATAG
GAAAATGATTTACAAAGAAAAAAAGGAAAACTGAACTTAGTATCTTTCTACTTTAAAGGGGTTCAAGAACTTAGGAGTAGTTACACACCTCCTA
AAATCTTGAAAAAGAGGCTATGAACTCACTTCACAAAGGGTTCAGATACTCACATTGTGTCCAAGAGACTCAGCAATTGGGAATCTTGCCAGTT
AGATTCATAAAGCAATCCGAAAGCCAGAATGCAATTTCTTTAGGGGTGGTTTCTAGATGTTTATTCTAGATATTTTTAAAAGATTTTGTTTATT
TTTGATGTGAAGAAAGAAAATTATTATAGAATCTAGAAATAAATATTGGCTTTAAAAGTATTAATTTGCCTTGTCCTTTGTAATTGTGATCCGA
AATATCTCACAATAATTTTGTTTCAGTGTTATTTTCTTCTCAGGAGGAAAGGAATGTTAAAACCTTACTAGCAACTAACACTTGATAAAGATTT
AAACTATATAACAATTTTAAAGAAAGTCTAAAACACCAGATAGTCTTGAAACCTCGCATGCTTCACCGTTTAATAAAATTATGACTCATTTTAG
TAAGAACATGCTTTTTTAGTAAGAAAAAATGCTTTCTGAGCTAAGTTTGAACTGCTGGGGCACATGGGACACCCACAAACCCAGTCAGCAGAAG
TCTCAAACTCCTAAAAGATTAAAGGAAAAATACATTAGTGTGTACAGAGAATGAAAAAAAAATCCCCAATGGTTCTAATCTATAAACGGAAGTA
AAACTAAAACTCCCCTAAAATCCCCCCAGTCATCAATCCTTATAAATTCAGCACTTATCTTCTTCCTTTACTTCTGGAGACCCTCATTAAACAA
CACGAAAGCCTTTGCTGACTTTTATCTCGTAAAATAGAGCCCTCAAATGACACTAAGCAGGGATACCGACCTCCAAAATCACAAACTGTTTTAC
TTCTAAAACCATGCATGCAAACGTGGGGTTTCCTGAGTTCTGCTAGGGGCGGGCTGTCTGGACCCCCGTGTATTTTTTTTTTTTTTTTTTTTTT
TTTTTTTTTTTGCAGGTGTGCAATCCAGGGTGCGCGCTCTGTGCAGGGAGGGGAGCAGAGGTGTGCGGTGGGCACCGCCTGGGCCGGGCGGGGC
GGGGTGGGTGGGGCCCGGGTGTGTGCTGGGGTGAGAGTGAAGGACATGTGACAATACGACGGTGGCGGCTGCAGGATCCGGGAGGGGAGTGAAA
GCTCCGCTGGGGCGCTCCATTAGTGAGCGGTGATGGTTGCCGCCCACTTGTATTGAAGCGTCCTTTGTCACTAACAAGTTAAATTAGAGATGTT
ATTTATTTAAGAAGAAGGAAAAAAAACCCTAGCCAAACAGCCTATGAATACATATGCTCACATCCCCTACTCCTCCAACTCCTAATTTCCCCGT
CTCCAGAGGGCACAGTTGTAAACCTTGACGAAAATCCAATCTTCTGTGCGGGAATTTCCCCCCACCGCTTGCCGCCCCCGCGACAGTGAGTGGG
AGCTGGAGGAGCTCTGGTCCCGCTGCCCGGGAGCACGCGGAGCCGGGCGACCGCGGTGCGGCAGCCAGGGAGGAGGGGAGGCGGCGGGACTGGG
CGCGGGCGGGCCGCCGCCGCGACCCGGGAGCGGGGTTTGCTCAGGAAAAGGCGCCGTCGCGGCCCCCGGCCACCCCTCCCTGGCCCCGGGCTCC
CTGCCCGCGCGCCTCCCGGGCCTCGCGGCGCGCTAGGCGCACCGCGGCGGCGCGAGCGCCGAATGGGAGCGGCGACCCGGCCAGCCCGGCAGCC
CCGCGGGCGGCAGCCAGGGCGACCGCGGAGGCGGCGGGCAGGGCGCGTGCGCACTGCAGGGGCGCCAGATTTGGCGGGAGGGGGAGTGTCCAAA
GCTCTTTGTTTGATGGCATCTCTGTTTACAGAGTTTACACTTTAATATCAACCTGTTTCCTCCTCCTCCTTCTCCTCCTCCTCCGTGACCTCCT
CCTCCTCTTTCTCCTGAGAAACTTCGCCCCAGCGGTGCGGAGCGCCCTGCGCAGCCGGGGAGGGACGCAGGCAGGCGGCGGGCAGCGGGAGGCG
GCAGCCCGGTCGGTCCCCGCGGCTCTCGGCGGAGCCCCGCCGCCCGCCGCGCCATGGCCCGAAGACCCCGGCACAGGTAACGGGGAGCCGGGCG
GGCGGCCGAGGGCGGGGGCGGGGGCGCGCGGGGCGCCAGGCTCCCGGGAGCAGGTGGGAATTCGTTCCGGGATCATCTGAGGGGCTGTCAGACC
CTCCGAGGACCTGGAGCCCCTGCCTCGGCAGCAGAAGCCGCTCCAGAGACTGATGAATGGAAGAGTCTTTGCGGGAAATGTATCCGGACGTTGG
GAAGCACGCCCTGCGGCTCATTTTGCAAGTTGCATGGGGATACATTTTTCTTTAGGGGGAAAAAGAAAGCACGTTCCAGCCGAGGGCAGAGCGG
TGCCCACCAGCCCCGGGGAGCCCACCAGGTGCCTGGCTTGATGCCGCGGGTGTCGGCGAGGGATCCGCAGCGTAATTGCTGGATGCATTGAGAT
ATGTTTGGACTTGGAAGTGCAGAGCATATGGCAGATACAGGGAAAATGCTGGATTGTTTAGTAGCTGCAGGTAGTTCATTTAAATTTCATTCAT
TCTTTATGGAGGCGAGGACCAGTGTCAGCTGACAGGCGGCTGAAAAGCAGATTTTAGGGGGATAAAGGGGTCTCTACTTTTGCAAATTGTCTTA
AGACCTGGTTTTGGAGGTTCGAATCATTTTTCTCTGAAAGGCTTTTACTTCCACTGCAAACATTGCACGCGCACCCACACGCGCCGCCGTCGCA
CATGTCACTGCACTTAGGGAGGCTCGCCGGGAAGATGGGTGCAGTCCCTTCACATTTTTGGCCGGACAGATGGGAAGAGGGAGAGGAGGAGGAG
GAGGGAAATCCTCGTCCGAACTGTCAGTTGCTCGTTCCCCAGTCTCCACCGGCCTCTGGACTTTCCCAGCAGCACACGCCGGGGCTCCCTGCGA
GCGGCGGGTGCCAGGGCTAGCTCGGCAGCCGTCGCAGTCGCCACTCGGCTCAAGGGGACAGAGGCCGGCAGCACCCAAGGCCGCTGCCGCGCAA
CCGGGACGCGATCCCCGCGGGCTCTGCCCCCAGGCGAAAGGTCCCTGCGCGGCGCGCACACGTGGATGCGGCTGAGGTCGCCGCGCCTTCTCGC
ACCCTCCTTCAGCCGCCTTAGGTCGCCCCGGCCGAGGCGCGGTGACCGGACAGATCCTCGCGAAGGAGTTCTAAGGCGAAGTCCATGGAGGCAA
AGCAAATCCATTCGCCTTTCCTTAGATTGTTTGATTTTCTCCTACTGGTTTGTTGTTGAGCCTGCGGGCGCTGGTCCCCGCGCGGCGCTGGGAA
GTTGCAAAGAGCGTGGGTGGAGACCCTGCAGCAGCACCCGCGGGCTGGGCTGGGCGGGGAGCAGCGCCGGGGCTTGGTTGGGCTCTGGGGACGA
GAGGGCGACTTGGGGGAGCTCCGGGCTCCCTATGCCTACTCCGGAGCCTGGCCGCGCAGGACGGTGTGCTGCCCCTCGAGGGCTCCACTTTTCG
CCTTTAGGACTTCACAGGACAGCTACCGGGGTCATCTGTTTTTTTTTTTTTTTTTTTTTTCGAGAGCTGAAACTGGCAGCAGGAGGAGGGAGTC
GGGCAGGGGTGCTGGGAGGAGGCGCGGCAGCGGCTCCGCGCCCCGAGGTCTCGGCCGCCCCCGCGGGGCGGGGCCGCGCAGGTTCCCTGAGGCT
CCAGGTAGCCCAGAGGGGAGGCGGCTGGGAGGGGCGGGAGCCTTGAACCTGCCTCAGGGGAAGAGAAACCCGACTGCGGGCTCGGAGCCGTTTC
TTGCAAGGGCGAGCGTCAACTCGGCAGGGATTATATTAACCAGGTCAGCGAAATGAGTTTGAACCCCTCCGAATCACAGTAGCGGCCACAAGGC
ACTTCCTCCCGCTCCCCTCCCTCTTCCTAACCCCCAGCACCTCTTTTCTTTTTCGTTTCTTTGCACGGTTTGTTTCCTTGACAGAATGTAACAT
TCATAGTTGTACTCTTGTAACCACACCGGGTCTAGCAGGTGGGGAGTCGAGTGCATCAACCCTGAAATCAGCCTTTGAAAATCCTCTGACTGTT
GGGTCTGAGATCTTGTCAGAGCAGATAAAGGAAAAGTTATTTCAGGTTCCTTAGTAGAAATTAATCATGCTTCTAGGACGACTTAGAGCAAGCA
AATCGCATGCAAAGTTCAAAGGCGAGCCTGGGTGGCCAGCCTGGCAGGGAAACTCAGGAGCTGGGCGCGTGAGCAGCCCCGGACTCGCCCTGCT
GCGTCCTGGCCCCTTCAGGTCCTGCAGAAGTCACTCTCCACCTTGCCCCCAAGACCTTAGTTTTGCACATCTTTGCCTCTGTGCTTAGCTTTTA
AGAGGCACGGAAGGACATGGCATGGTCATGTATTAGCGCTGGAAAGTACCTTAGAAATAGGATCCAAGCAAGCCCTTATTGTAAAGGCGAGGAA
CTGAAGTCTTGAGAGAGAGGAAGTGATTTGTCCAAGTCATACAGCTAGGTGGAGGCTAGACTAGAACCTTTGCCTCCAGAACCCTAGTCCTGTT
GATTCCCGTGCTCTCACCTGACGTTGGCTGCCCCCTGCGGGCGGCTGAATAGGTTGCTTGTTCCTGGGTTGTTAAACCGCTGCGGAGAGAGAGG
TGCTCAATGACTACTCTGAAAAATGTGCTCAGTTCTGAAAATGTGACCTCTAGATCCAGCCAAACCTGAAAAGTTAATTCGACTTGAATCTATG
ACTGCAGTTTCAGATTCCTCTGGTTTTCTTGGTGTCTCTCACATTTCACAAGGGACTGCATTTGGGGTCTTTCCAAGGAGTTCCCTATGAATCT
TTCAAAAGATGCAGACTTACACCTCTGGGCTTTATAGCTTTTTTTTTTTTTTTTTTTTTTTGCTTTATCATATCGTGTTTTACATTTTTGTAGT
TCTTGTTCAGAAGTTGTTTAGTGCCTCCTGTTGACAGTGGAAACAGAACTAGGCTATTGTTTCAAAACACAGACACTAAATGAAAGGAATTATT
ACAGATTATAAATATATATTTCCTGGGATTTTATTTGGTGGTGGTGGTATTTTAAATTTGGATTACATCATGTCGCCATTCAACCTGGACTATC
AGATTGTTGCTTAAAATAATAGCTTTAAATTATGAGAACCCCCAATTCAGGATTTTTGGTAAGCACTTCAGCTTCACTTGTAGGGGGCTGGTAA
TATGCATACCAGTGAGTTAGTTTGGGAAGACAAAACCTGTAATGAATATAACTTTTCTTTTTAAAAACAATGATTTACATTATGATATGGCTTT
ATATCATTGTTTTTATTGGTCTTAGTTTAATGGGTTTCCCAACATTGCAATGACAAATTTTAGTGAAGTTTCTAAGATAGTATTTTTCCTATTT
TAAGTTTTATAAGAAAAACTATAAAATAAACCGTGTCTGTAAAATTATTGTCATCTGATCAGTTGATTAGATTAACAAACATTATGATTATGAT
TAACAAACAGTAAGCATATAGTTACTTCTTCAAAAGTCATAAAATTTCCTAGGTTAGGTTAACTAAATAAAAGAAAAAGCTTTCGAAAATAGTA
ATGGGTTCTTTTAAATCCATGGCTTTTTTCTGGGGAAATCCCTTCTGGATTAGGTTACATTTTATAATATTTCAAACACCATTCCATCAGAGAC
AGAAAAAACAAGAGTAAGTAAGACTCTGACTAACAAGTGGCCTAATTATTCACTTAGTTACTCTAGAAACTAAGTATTGTAAACATGGGCACAA
GTTGGATCAACCAGGCCTGGAGTTGTGAGCAATTTGGTATTAATTTTATTTACAAAACATTAAAGCTTGATCACTCAATGTTCTTATCTTTGCT
TTGGTTTTAAAATCCTTTCCTCTTAGATTCTCCTAATCCTCTAGACTTTATGGGATCACTATAATTCTGTTTTGCGCTGTACTACTTCTTGATT
TTTTTCTTCTTTTAATAAAACAAAAACCCCATTGGAATAGCATAGTTGAATTGTTTATTATGTTTGAGAAATATTATTTAAACGATGTGACAGA
TGCCAAAGATTTTGAGTGTGCACTTATATAAAGGACATGGGTTCTTGTTCCTTTTCTTATCCTTAACCTTAAGTTTTCAACTTAAACCTTCACT
GGTTGGAAGGTGGCCAAATGTGTAACTTGTCCCTGGTCTAATAGTAACAGCAGGTTCAGACATGCAGGGGAATAGGAAGGTGCCAGGTCCTTGG
CCGTGTCTGTGGATACCCATAACAGCAGAACCAGTTTACAATACTAGAGCAACAGAATGCAGCAAACAATCTTGTTGTGCAAGTTTTCAAAGTT
TTGTCTTCATAACCTTTGAAAAGATTGTTGAGGAGTTTTGTGTAAGTTTTGTAATCCAGTAGTAGTCTAAATCCTCTTGTTTCAGCCCACGTCT
ACCCATTCTTATTTCTGCAGCATATATAGCAGTGACGAGGATGATGAGGACTTTGAGATGTGTGACCATGACTATGATGGGCTGCTTCCCAAGT
CTGGAAAGCGTCACTTGGGGAAAACAAGGTGGACCCGGGAAGAGGTAACTAATCATTTTATCAAGACGCAGAAAATAGATAGAGTGTATAATTT
ATAAAAAACAAAATTTCAACTAAACTACAGGTGCTCAAGCTCATTAGCAGGCTCCTTGAGATCTTATCCTAGCCCGCATGTGCAGCGTGCCCTA
TGCCCCCCACTTCATTATATTATCCATTTCCACACTATTTGCAGTTCTTTGAATGTCCTCTTCTCATTTTAGGCTTCATCTGTGTTTCCTTTTG
CCTGTTGAGTTCTCATCCTTTTGTCCCACCTCTTCTTCCTACTTTTACATTACCCATGAAGGCATCACCTCTTCTGTGAAGCCATCCCTGGCTT
CGCCGGACAGGATTGGATGCATCCTCCTGTGATCCCATGCATGGGTGCATGTTCTACTTTATTGTTGCTGGGACATATATACCTCCTCCAGCTG
ACTGCAAGTCTGTTGAGGGCAGGGCTCTTGTCTATTCATCTTTGTGTTTCCTGAATTCACAAATGCTTGGACCCAAAATAGGATATTAATAAAT
GTTCTCTCAATAAGTGAATGACTTGCTACTCCCAAAAACAAAACAAACAAAAGAATAAAACAAAACACTCAGAGAACTCATCTTTTTACAGTGT
ATTGATTTAATATCCTTGATATACTTAACAGAGCCATCTATATGAAGGTATGTTTTAAAATCATCCGATAATGGATGGAGACACATCTGCTTCT
CCTGTTTCTTTTTCGGCTGGCTTATCAAAAGTTTGATTTTGTAGGAATGGAAAAATATTTTAGAAGCAAAATTATTAATATGTGGAGAAAAGTT
TCTCTAATGTTCTTAAAATTTTTGCAACTAATTTTAGATGATTCATGTTACAATAAATGGTATTGGCTCTTCTTCAGTTTCACTGTTCTCTGCT
TCATTTAAACCAGAGCTCCTTTTAACAGGCAGGTAGGATTCCCAGAGAGGCCCATAGGAAATTCATATTTTACCTATTAAGATTATCCACTTGG
ATAATCCTTATGATACGGAAATTCTGTTGTCAACATTGGTTTCCTAATTCTTGTGTCTGTATTGTACCAGGGACCTCCACTGTGTGTGAATGTT
ACCTTTGCAGAAACATAAGAAGGAATGCATTTATATACTTATGAACTGGTAGGATTGTGAACAGCATATAAACTAAAAGAACTGATTAGCTGGA
AATGTTTAATGTCTTTTATGTTCTAACACATCTGGCCTATCTAAATCTAACTGATTTTCTTATATAGCTTTCAAATTTAATTTTAAAAATGGTC
TTTATTTCTTTATAAAAAGTTAAACTAACTGGAAATATTCTATTTCTTGCTCAAGATCCTATTTATTACCATAGGTAAATGTGTACTGAATCTA
GCACTGGAATGCATTTTTTCCTAACAGAGTAAAGCAGTTATTTATAATAAACACTCAATATATGCCAGGCATTATGATAAATGTTTTGCAAAAT
TGCCTCATGTAATCCCCAAATAACCCTGTTAATATCAGCCTCATGTTAATAAATTGCCCCATGTAATCCCCAAATAACCCTGTTAATTTCCATT
TTATAGATGGCAATACTAAGGCCCAGAAAGGTAAAATGGTATACCCGCTTTACACAATTAATAGTTGAGTCAAGATGCCAACTCTGGTCTGATT
CTAAATAAATCTATACTCCCAAGTCCGTATATTACATTAATATCTCCTGTATTCGGATACTTTAGAGAGACTTTTTATTCTAGATTCGTTCCCT
CATTCACTTTAACTTGAACAGAGTTATATAGTAAATAACTGATATCCTAGGATTACCTTTAAATGTCTTAGGATGAAAAACTGAAGAAGCTGGT
GGAACAGAATGGAACAGATGACTGGAAAGTTATTGCCAATTATCTCCCGGTAAGTTAGTAAGTTTTTCTTATAACGAAAGGAAAGCTTCTCCCA
AAGATTTCTATAAAATATTCTAGGAGGAGTTATTAAGATAGTGTATAATTTACTCACATTTAAGAAGTGGGAGAAATGCCCTACCCATAACTTA
TATTTTATATTATAATGCATATAATTAAATTATTAGAATTTAGATTATTAGAATCGATTATTATAATTTAGATTATCAAATTTTAAAAACAGTT
GCTTCTTGTTTTATCTTTTCATTTATAGGATAATTAGTATTTTGATGATTTGTTACATGGTAGTTGATTAAACTTGATATTTCCTCCAAGTCAG
TTTTTCCTGAATTCCCTATTTTTGTCAATAGCAATACTTTTGGCACCAAGGTTTAAAATTCTGAAGACCTTTCTTGACTTATCACATTCCCCCA
ACCCCTACATCTAAGCAGTTACTAAGGACCCTGGAATTCCTTTATAGTCAGTCCCGTCCTTTTCATTCTCATGGCTGTGCTGAAGGTTGGGCTC
CTGCCCTCTCATTTGGACTATCATATAACTTCCTAACTGGCTTCCCCACTTGTAGTTTTCCATTTTTCTTTCTTTTTTTTTTTTTTGTTTTTTG
TTTTTTGTTTTTTGAGATGGAATCTAGCTTTGTTGCCCAGGCTGGAGTGCAGTGGCACGATCTCAGCTCACTGCAACCTCCGCCTCCCAGGTTC
AAGCGATTCTCGTGCCTCAGCCCCCTTAGTAGATGGGATCACAGGTGCCCGCCACCATGCCTGGCTAATTTTTGTATTTTTAGTAGAGACAAGG
TTTCACCATATTGATCAGGCTGGTCCCAAACTCCTGACCTCAAGTGATCTGCCCACCTCAGCCTCCCGAAGTGCTAGGATTGCAGGCATGAGCC
ACCGTGCCCAGCCAGTCTTCTACTTTTCCATTCATCCCAAACACCATTGGCAGGTGGAACTTCCTAAAATGCAGCTGTACCTTATCTTTTCAAA
ACCAACCACAGTGACAGAATCTCACAAATTAAGTAAACTGCCTTGACTTGGCACACAGAGGCCCTCCTCTATTGCCCCAACTTACAGCTCCAGT
TCTGTTTCCTGCTTCCTCTCTCTAAGCAGGTTTGCTTGAATTTATTTCTGCTTTACCTCCTCTGGGCTTTTGCTCCTGGTCTTCCCACCTCCTA
GAATATTCTCCCTTTATGTCACCAATTAAAGTTCTGTCCATCCTTCAAAGTCCTTATGAAGAGCTCTTCAAGACTCCTCCCAAGGAAACCATTT
CTCCTCCTAAGAAGATGCTTCTCTCCTCACTTCAGTATCATTTTGCTTTGGTCTTTTCTTCCAAGTGAATCAAATTCAGCATCTGTGATAGTCA
GTCATCTACTTGTCTTTTCCTTCTTATAAGTTTACAAACTCCTGTAACATAGGACCTATCTTTTATGCATCTTTTTTCTTTCTGCAATATAAAT
ACTTTGGTCCTGGATTGAAGCACATCTCTTGTCTGAATGGGAAGCAAACTTTAGTCTTTTAGGGTGGTTTCCATGAAAGCCTGACAACATAGAA
TCTCTATCAAGCCTCAGGCTATGTAGATACTCTGCAAAAGTATCTGAGGTTAAGGTTAACTTGGCAGGCACATGTGTAGTATGATGGTCCTTTA
GATCATGTGGCAGGAATGTTTCCCTCAGGAGGCAGAGTGTCTAATTTTAGATGCAACTTTGATATATTACTTGCATGTTCTGTAGCCTGTAAGT
AATGAGGGATAGATGGGCTCTAAACATGCTCTAAAATGTTCTTAAATATTTTCACTGCATATGGTCTATGTGTGATTCCTATTACAACAAAAAA
TTCACGTGCTTATCACATCATATCTTTATGGTGGTGCATACTTTATTTGTCTAGAATCGAACAGATGTGCAGTGCCAGCACCGATGGCAGAAAG
TACTAAACCCTGAGCTCATCAAGGGTCCTTGGACCAAAGAAGAAGATCAGAGAGTAAGTTCTTTCTTCATTGGTGTGTGACTCATAATTAAGAA
TATTCCCAAAATGCTAATATTTTCCTATTTGAGGAAGCAGGTAAAATGGGCAAACAGGAAGTAGAGGACTCTATTCCCATATTTCCAGTGAATG
AAAGCAAATTTTGGAAATTTTCTAAAGATCTTGTAACACTGAAGAATGATTATACTGACTCATTACATAACTTTAAAACATAGGTTATTTTTGT
GTGTTTATCTGAAGGTGATAGAGCTTGTACAGAAATACGGTCCGAAACGTTGGTCTGTTATTGCCAAGCACTTAAAGGGGAGAATTGGAAAACA
ATGTAGGGAGAGGTGGCATAACCACTTGAATCCAGAAGTTAAGAAAACCTCCTGGACAGAAGAGGAAGACAGAATTATTTACCAGGCACACAAG
AGACTGGGGAACAGATGGGCAGAAATCGCAAAGCTACTGCCTGGACGGTAATAATATGTCAAAAAATATATTGATCGGGAAGAATGAGGGAGTG
GGTATTGAACATTCTGCTTTAAATGTATGGTGAGTAAATTATATTTCATCAGTCGTAAGTGTTTTATTAGATAAACCAGCTACATAGCTTTTAG
AGATTGAAGACATCTTAGAGTTTATTTGGTTGAGAACTTCCAGGATCCACCTGACAGTGAGCCCTGGGTGAAGACATTTAGCTTTCCCCAGCCT
CAATTTCCCCATTTCCAAAATGAATCATACAGGGCCAGTGTTAGGATCAAGTAAAATGAATGTCAAAGGACTTGGTAAACTATAAAACTCTAAA
TGCATAGAAAGAAACTGAAGACAGTTTGTCTCTGGCAGTTCACGTAGCTGGTTAAGTTACTCAAAGGAACTCAGCTACCATTTTCCTGATAACC
ATCATTCATTTCTTTTATTTTTTTTAATTATTATTTTTTAGAGACAGAGGCTTGTTCTGTCACACATGAGTGACAGAGTGCAGTGGCATGATCA
TAGCTCAATACATCCTTGAACTCCTGGGCTCAAGGGATCCTCCCACCTCAGCTTCTTGAGTAGCTAGGACTACAGGTGCACCCCACTACACCCA
GCTGATTTTAAAATATTTTTTGTAGAGATGGAATCCTGCTATGTTACCCAGGCTGGTATCAAACTCCTGGCCTCAAACAATCCTTTCACCTCCC
AAAGTGCTGGGATTATAGGCATGGGCCACCATACCTGGCCACCAGTATTCATTTCATTATACCACTTTGCTTAATTTTTTACTTCATTGATTAA
AGAAATAATGTATTTCTTGAATTCTCATACTACTTCCAGGTTTTTAAATAATGTCAGGGTAATAATGCTTTCTTTTTAATGAACAAGGCAAGAA
AATGACCTCATTATGAGGGATAACTATGGAAGCTCATTTTGTCCCATTTTTCCCACTTAAGTAGTATATAACAGATTTTGTTCCTTTGGAAAGC
AATACTTTTAATGATAAATCTTGCATTAAAATGATGTAGATCAAAAATATTAAAAATAGTTTTATTCTTGAGCTTCATAATCCAGGAGAAAGTT
TATATAGTATTCTCTAGTGATGCTCCAAAGCATTTGCACCCTTGTGGTTCAGTATAAACTTAGAAATTTTAAGAGGTTGGTAAAAAAAGGCCTT
TCCTCCCATATGGCCTGAGAGGATACACTGCTGATTCTTCTCAAAATTAACAGGGCTATTGAACTTCCTCTTTCTTTAGCATACATTCCCCCAC
CCCCATTGAAATAGAAAAAGGGAGACTTAAACGGTTTTTTTGAATTGAGAAACTGTTTTCCTGAGAATGGAAAGACTTTAGTAAAAATGCGTTG
CCTTTTGAAAATATACTGGGGGTTAGGATCTTTCCCTTTGGGATCCCATTTTCCACTCTCGTCTCTGTCTGATTCAGAAAACAATTGGGGTGGC
TTTCAAAGAGTAGGAGGTTTTTTGGTCTCAGAAGTTCTCACTAGTGCTTTGAGAGATAAAGAGTGAAGTATTCTTCATCAGCAGCATCTTATCA
TCCACCAGACTTCACCATGTCCCTCTCGTGGTGTGTAGGCCCTGCTGTGCCACATTCAAAGGAGTAATTTCCCCAAGGCAGCTGGAACTTTACA
TTGTGTTGGAGTCTTGTACCGCGACCGGTCAGAATAAACATGAAGCGTTATCGTATTGAAAATACTCCTTTACCAAGGAAATGGAATGGTTAAA
AGATACTGATATTTCTAGAAAACGACATGCATTCCAGATTGTGTAACTAAGAGAAATCTAACTAGGCAATAGAGAAAGCCTGATCTCAGAGGGT
GGAGAAGAAGGAAACAATTAAAAAGAGAAAACAACTAATAGGCCCAAGGGAAAGGTCTCTGTTAGAAACAGTTTTCTGTCAATTCACTTTAATC
TGAATTGAAATTTTTGTTTGTGAAATTTGTGTGATATATTTCTGTGCAGAACTGATAATGCTATCAAGAACCACTGGAATTCTACAATGCGTCG
GAAGGTCGAACAGGAAGGTTATCTGCAGGAGTCTTCAAAAGCCAGCCAGCCAGCAGTGGCCACAAGCTTCCAGAAGAACAGTCATTTGATGGGT
TTTGCTCAGGCTCCGCCTACAGCTCAACTCCCTGCCACTGGCCAGCCCACTGTTAACAACGACTATTCCTATTACCACATTTCTGAAGCACAAA
ATGTAAGCCATTCCTGTGAATCTAGTTTAATGAGAGAGACGGTTAGTTTAGCTCCAGGTAATAATCATACTTTGCAGTTGTATACTTTTCAGAG
AACTTTCCTGAGCATGATCATGGCCAGTCTTCACCTATGCCCTTTGAAGTAGATAGGGTGTAGGTATGGTGTGTTACCCCTGGTTTATGTCTCC
AGAAACGAGGATGGTAAGCGCTTTGCCCAGGGTGACACAGCTCATAGAGATAGTGCTGACTAGAAAACGAGCTGTCCGATTCTAAATTGCTGCT
TCAAATCTAATTTGAAGGCAACCTTTGAATGTAAATTTACTTAAGGTACTATTGAAGAATAGGGGAATGGAGTCGCATTTGTAGATGTAATGTT
TTTCTCTTCCAAAACGAAAAGGGAAGGTAGTATTATGTTTAAAGAGGAGAGAGTTTGACACTGAAATTCAGTGGTTATGGTATGTTTCATTCTG
GCAGTGATCAGGCAGACATGTCTTAATTTATTCACTCAGTAAGTGTTTACCTTCTTACTCTGAGTCAGGAGCAGAACTTTGTGACATTCATTTT
CTGATGTCTCCTTTTAGTCTCCGTTGGATCAGTGTGATCCCTTCCTTATTTCTAGATATTTATATTTAGGGAAAGTGATAATCCATAATAGGTG
GAAAAAGAAGAAGAAAGAGGAAAAAAATCGAGAAACTTTAATCTTAAGAATAACAATATTAAATGATAAAACTTACCCAAGAATAGAGAGCTCA
TATGCAAATTATATGCAAAACATGTTCATAAATAGTTTTTATTCTTCTCTGTCCATGGATTTGACTACAAACTTTACAGAAAGGTCTCAGATGA
ATAAATTATCATAAGAATTCCTGTGCTTTTTATTAAAACTCAAAATTATTCCAAGAAAAATATTTTTATTTACAAACAGCTGGTGCCATCAATT
TTTCCCATAGAAACAATTTTGAAACATTCATTAATGTATTTTATAATATATTTTTTATTGTTTTTTTCATTGTGTTTTTCTCTAATTAAGGAAC
ACTTCCCAAAATTTAAAGTGCTCATCAACACCTCCCAAGGATTTTTCACCATGTTCCTTTAGCTGTTTAGCCATTTTAAAGGCCTGGCTAAATT
TCACATTAAAAGGTTTTAAAAATTTGTTTAGTGTATTTTATCAGTAAAGTGTTTTTTTTCTCTCAGTCCCAGAACGAAACCTCAGGAAGCCAAG
TCCCCTGAAGCATATGTAGCCCTGAATGACGTGGCCTTTGTTTTGTCTTTTGCATCTAGGTCTCCAGTCATGTTCCATACCCTGTAGCGTTACA
TGTAAATATAGTCAATGTCCCTCAGCCAGCTGCCGCAGCCATTCAGGTAAGATCATTGATCATTCACTGTTCAAAGCACCACTTTTAAGAAATG
CTTATTTCTTTCATCTAAACACATATTTTTACCTGAGCAACTAAAGTAAAAAGGAAAGGGAACATGGAGAGGAGAGCAGAGTCTTGGCCAGCAA
TCTATTAATCTTGTCTTCTAGAATGAGTTTGCTCTGGAATTTTCTGATTCTTTGATGTTGCAAGGTGAAATAACTCATCAGTCAGCATCCACTC
TAGCCTGAGATTTCACATCACTCCCTTAAGTCTGAAATAACATTCACAAAGATTTCTGCAGGAGCCAAATTTGGTTGGTGGTGTTGTCACAGTG
AGGCTCTTTGGGCTGTGCGGCACCTCATGACCATATTGGCTACACCCATTGTATTTGCAGCTTCCAATCAGACCTTTGTCTTTATTTCCCTTAC
TTTGTAATTTCAGAGACACTATAATGATGAAGACCCTGAGAAGGAAAAGCGAATAAAGGAATTAGAATTGCTCCTAATGTCAACCGAGAATGAG
CTAAAAGGACAGCAGGTGCTACCAGTAAGACTGTCATCATGTGCTTGAATGAGGGGATAGCAGCTTTGCCTCAGTTTACCTAAGCGCTCTTCTC
TTCTAAATATTACACTTAGCAAGGCTCCATATATCCATTCAGAATGTCTCAACACAAGAAGTTGCTTGTAGTAAAATGTAGTTGGTATCAGATT
ATATGCTGATTAAATTGGAAGCAGTCTTTTTGTAATTGCAATAAAAATGCAATATCCACTTATGATTGTTATCATCAATTTTTTTCTTCAGAAG
GACTATAATCAGTTTAAAGACTAGCAAAGATAAAATGAATGAAAATCTGCATTTTCTACAGAGCTATGTATCTATATGGTACCATTTTGGTTTT
TTTCTATTATTTTGTTAACCTATATTTTCATATTTTATAATTTCAGACATGATTTGATGCATGTTCAGTATATACATGTAAAAGGTTATATATA
AAACCTCTTCATAAATAACATTAGCTGCTCTCTTTTATTTGCATGTGTGAAAGTTATGTGGGCCATTACTGAATTCTGACATCTTTAGCGACTG
GGCAGCCAACTGGGATGGCTCCTTGTGCTTTGCAACATACATAGTTAACCAACAAAGACAATAAAAGAGAAGTGATGCCCAGTAGGTATTCCTA
GGGGTAAATTTATTTTTTGTTAAATAATAAGATGTTTATGTGCACATGCTAGTTCGACCACTTTTTAACTTTAGTTAATGTTTTGCAATTTCTC
CTGTTGTGTGGTCATACTGTAAGGAGACTTCTGTTAATTTGCTCTTCTGCTTTCCTAACCACGTTGATTTGGCAGTGCTTCACTGCATCAGTGC
AATCTGTAGCATTAGAAAGGGATAGAAGCAACAAGAATATGTGTAGATCAAAGCAACTCTAACTCCTGAGTCCTCTAGCTTTTTTGTGGTTAGA
ATGCAAATAGGACAGTTCTTCAGTTCTTCACTCACAGCAAGCATTTATGGACCTTTGGCAAATTATATTAAAGTGGAATTTTAAGACAATATGA
TAATTATACTTCGAAATATAGACCTATGATTTGACATTTTTACAAAATGATAACAAGGTAATCATATAGGTACAACCTCTAGCACTATTAAAAA
CAGGGTCTTGCATTGATAAAAGGAAACATCTTGACAACTGTTTCATAGGCGTAAGTTTGGGATGATGCAACTACTCTTATCTTTCCTCCAACAG
CATCTGATACCTTGTGCAACTTCATTGCTAAGTTCCTTCTCCCTTTCTTCTGTCCTCTCTTTATTTCTACACCCTTCCCCCTTCCTTAGACACA
GAACCACACATGCAGCTACCCCGGGTGGCACAGCACCACCATTGCCGACCACACCAGACCTCATGGAGACAGTGCACCTGTTTCCTGTTTGGGA
GAACACCACTCCACTCCATCTCTGCCAGCGGATCCTGGCTCCCTACCTGAAGAAAGCGCCTCGCCAGCAAGGTGCATGATCGTCCACCAGGGCA
CCATTCTGGATAATGTTAAGAACCTCTTAGAATTTGCAGAAACACTCCAATTTATAGATTCTGTAAGTAGAATTGTGAAGGGAAGTTAACGATT
CTGGAAGATAAATTAGAAAACCCATTTCTTAAATCATTGCCATTTTCTATAGCCAAGCTGTTATTAGCATATATTAATGTAAAGGTAGAAGTAT
GATTTTCATCTTCATGAATATGTTTTTTCAAAGATCTGATTTCATAGCCTACCGAGAGAGACAAAAGTATTTATAAATACTTACAAAATAGGAC
TTTTGAAGGAAGAGTTTTTTTTCACATTTTCACACTTTTCCTTCAAAAGTCATATTTTTAAAAATAGTCAGATTGATGACTTATAAAGCAGAGC
TTGGATTATGGGAACGGAATGGTGTTTGAGTTACTTTTAACTTTTTTTTTTCTTTGCTTACTTCATGGTGCTTCTCTAGAATTTTGTCTTGCTT
TTGAACAGCACATTAATGATTTGGCTTACTCAGAGCTGAAAGCAATCTTTTAGTGTATTTGAAGTAATAATCAGCTCCTTGGTCATACCTGGCT
CTTTCCACAAAATGGTGATTATTGATTACTGGTCCCATTGCTTCACTGAGATGAATCATCTTTCGTGAAACAAATTTGTATTCAGCACCTCCTC
AGCGAACACTTGGTTCCTTTCCCAGTTGATTAAAAATCATTTATTGCACACTCATTGGTTAGGAGATGACCATTGCCGTAATATGCCATCTAGA
AAAGGTCTTCATTTTGCTTTCCATTGCATGCAGATGTAGAGTGTCGGGAGGTCCCTGCAGCACATCAGAGGGCCAGCCTTGAGGCAGCCCACTA
GACCCTGCTCTACTGCAGTATAATAGAGCAACTGCTCATCTTGAAGCTGTTGCTCAAGAAGCCAAACCAACGGGCCTAGTGTTTGCTTTGCGTT
GAGCATCTCTCTCTCAGTGCTGTTTCAGGTGATGATGGCACACACTATCTCAAAGTTCTGTGCCTCCCACATTGTTTCAGGATTCTTCATCATG
GTGTGATCTCAGCAGTTTTGAATTCTTTGAAGAAGCAGATTTTTCACCTAGCCAACATCACACAGGCAAAGCCCTACAGCTTCAGCAAAGAGAG
GGCAATGGGACTAAACCTGCAGGAGAACCTAGCCCAAGGGTGAACAAACGTATGTTGAGTGAGAGTTCACTTGACCCACCCAAGGTCTTACCTC
CTGCAAGGCACAGCACAATTCCACTGGTCATCCTTCGAAAAAAACGGGGCCAGGCCAGCCCCTTAGCCACTGGAGACTGTAGCTCCTTCATATT
TGCTGACGTCAGCAGTTCAACTCCCAAGCGTTCCCCTGTCAAAAGCCTACCCTTCTCTCCCTCGCAGGTAGAACACAATTTCTGCACAGCTGTT
ACTCATTTTCAACAGACACCTGAGCCTTAATAATCTAGAAACTAATACTTCGGAACAAATGACTAATTACTGCTGCCTTTGCTGATTTCATTCT
GTCGTTGAATCTAGCTGTTGCTAAGTTATACTGCCTCCCAAAGTTTAGGCTGTCTGCAGGCTAACTGTTGATTTTGGCATCATTCTCTCTCTTT
TACTTTTGGGTGGTTTTGAAAGATAGTTGCATGAAACGTACTTTTATTTCCTTCAACACTAGAGTTTAACATTTCTGTCCCTTTCTTTTTCTCC
CATCTGATCCCTCCCTTTAAAATTGTTAACATCATTTCTTCCTCGTTGTTCATTTATTCTTATGCGATTCACTGACCCAGGGGAGGAGGGAACC
AAATTGTGTTTATATAACATTTGGGTTGCAACTAGAAAATTTAAAGCTAGAAAATTTAAAGGTTCCAATTCATCTTTAAATGGAAGAGTTTGGG
AAGAGGATGAGAAACAAATGCTTAAGGAAAAGAACATATTTTTAATTTCAGTTTCTCAACATTGGCTCTTTTGAGTTGTGCCATACTATTTTAC
TAGTGCAGTTTTTGCCAAGGAATATTTTTATTTCATACAGCTTTTTTAAGGCATGAAAATCACAATTCTTATCAGAGATTTGTGTTCCTAAATT
ATTTAAATAATTAAATTAATAGATAATTTGGCACTTTAAAACATTTTTCCAATTGTATCTGTTGAATTTGTTTTAAGAACAGATTTAGGTATGA
TATAGGTATATACATTTGGGAAACACTGAATCAAACAAAGTTACAAGTAGGTTTCTTAGCTGGGCATAGTGGCATGGGCCTGTAGTCCTAGCTA
CTGGGGAAGCTAAGGCAAGAGGATTGCTTAAGCACAGGAGTTCAAGTTCAGCCTGGTGAACATAGCGAGATCCCATTTCTTTAAAAACTTTCTT
TTTTTTAACTGCAGGCCTTCTCAGAGTTATTAATATGCCGTTGTGCCTTGTGAATTTCCAGGAGGAGATTATGATATGTTGTGTTTCCCAAATT
TATTTTACCTTGGAATTGTTTAAGGAACACCTAGTTTTTTCCCAGAACTTTAGTGTTCTGAGAGACACAGTTTATTATTCAAGAATTTTCCATA
TAATGTAAACCTTCTGTAATACTAATTTTGGTTCATAGATTATTAAAGAGAAAAAACAAAGTATGAAAGGAAACTATTAATTATTAACTATTAT
TTTCCCCAAAATCACTAAAGCCTTTTAAATGTTAATAAAGAACACATCTCATTCTCTTTAATGACAAGTGTCATTTATTGAGAGTATTTTCTCA
CAGTTTTGCAAGTTTTTCAGCAATGACCCTAAATTTTATTTCTTGGAAACCACACCCAAATCCTGGGTGCTCAATTGAAATAAAACAATACCCT
AGTCAATATAACATGCTTGTTAGCATCTTTATTATTTAATATAGTGGGTCAGGAAAACATTCTTGTTATCTAGGTGTGATTTTTAAATTGGGGA
GAATAAAGAATTACCATCTAATCTAAGTATTTTTTCTTTCTCTCCATATTTAGTTCTTAAACACTTCCAGTAACCATGAAAACTCAGACTTGGA
AATGCCTTCTTTAACTTCCACCCCCCTCATTGGTCACAAATTGACTGTTACAACACCATTTCATAGAGACCAGACTGTGAAAACTCAAAAGGAA
AATACTGTGTAAGTCTTTGGTTCAGGAATAAAATGATTTATCTTATTTACTTATATTTAATTAATGGAATATAGTTCCCAGATGTCTGATCCAC
TTGTATGTGCATAATTTTACTGACCATTTATATATAATCATGTCTATTCCCACATTGAATATATCTGTCCTGGGCATATTACAACTACCTACAG
GTAGAAGATGGGAAGATAAACTAGATTCCTTTTGTAACTTACTCCAAAATTTCCTATATATGCACAAGTAGTTTTCATAAAACTTTTCCATTCG
GTTTGTTTGTTCCCTCCATAAGGCCTTCTTATGAAAGCCAGATTTTCAACCTCCTTTCCTCTCTAATTGTAAGCTTCAGAATAAGAACGAGTAT
GGTTTCTTACCTAGTTTGTATTTTTATTTAATTTTATTTGTTTATTATTTATTTATTTTTAGTTTGTATTTTTTAATCTTATTTTTTCTTTTTG
AATTTATGTGTAGATAGGACCTCTTCTGACATCCCCAGGAATATTATATGATTAGAAGCCAAGGGATGAGTAAGCAGTCATTTTTATTCAATAA
AGCCTTAATCAATTCAGAGAAATGGTAGAGAAAGTGTGAATTATCAGAAACCTTGAAAAGAAATGCACTGTTATTTCGAAGTACAAAAAGTAAA
TTTTAAAAGTTTAACATGTTTTGTAGTATGGGCTCTGTTTTCATGAATAGGGAAACCTGATTTGTAAATGACATCAGGTCTTCTTGATGCTCAT
AGTCTTATACAGATCATAGGCACTACATGTGTCTTCAGAGCCATTAAGGAAAGATGTTAAATCATTTAATGCTTTTAGTCTTCGAGGTATAATA
TTTTGAAATATTTGAAAATATGTGATATGCATATTCTCTTTTTCTTAACGAATAACGTGATTAATCAGCACACCCCAATTCCATATCCGGAACA
GAGTTTATTGTATTCAGTGGAAAAATGTTAAATGTAACAGGTAAAGCCATGTAGGGACATTTATTCTTTTGTATTGAGCCACTGCTTGTTTTCT
TTTTAAAGTTTTAGAACCCCAGCTATCAAAAGGTCAATCTTAGAAAGCTCTCCAAGAACTCCTACACCATTCAAACATGCACTTGCAGCTCAAG
AAATTAAATACGGTCCCCTGAAGATGCTAGTAAGTTCTAGAAAAGTTTTTGGATTTCATTGGTTTATTAGTCCCATTAGGAGTTCTCTCTGATG
CAAAAATACCCACTCTTCCGTTTAGCCTCAGACACCCTCTCATCTAGTAGAAGATCTGCAGGATGTGATCAAACAGGAATCTGATGAATCTGGA
ATTGTTGCTGAGTTTCAAGAAAATGGACCACCCTTACTGAAGAAAATCAAACAAGAGGTAAACACGCAACCCTTTGGAAGCAACAAGCTGAGAA
TCCTGCCCCCTTTATTCCTTGTGTGCAGCTTGATGTGTCTGCCATTGGCACTGTGCAACACCACGACTTTTCGTGCAAGATTTTCATACAAGGT
GCAGCGAAAAGGTACTGCCAGACTGTAGGCATCATCAACCTTTCCTTTGGGAAGCCAAGCCATCTCTATCTACTTCATGTTTGGACAGAAAACA
TACTAGAGAGTATCAAGAAAAGGGAGGGAAAGAGGTTTTTAATGTACTTTTAAACATTACTGATTATATAAAGTTGATATTTCATTTTTAGATG
TGAAGAATTGGAATTTAAATTAAAATAATAGTATCTTTAATTAATTGTATTAAAAAAAGGGAGGCTGGGCGCGGTGGCTCATACCTGTAATCCC
AGCACTTTGGGAGGCTGAGGTGGGCAGATCACAAGGTCAGGAGTTCGAGATCATCCTGGCTAACATGGTGAAACCCCGTATCCACTAAAAATAC
AAAAAAATTAGCTGGGTTTGGTGGCGGGCGCCTGTAGTCCCAGCTACTCAGGAGTCTGAAGCAGGATTATGGTGTGAACCCGGGAGGCAGATCT
TGCAGTGAGCCAAGATCGTACCACTGCACTCCAGCCTGGGAGACAGCAAGACTCCGTCTCAAAAAAAATAAAAATTAAAATTAAAAAAGAGAAT
CTAACCTGCAAGGATCAAAGTTTTAGCTTGTTGGCAACAGCTGTTTTCCACCAGGTTAGATACAGTGCTTCTTGAATTTATTTTCATGTAAAGA
TAAGTATTTTTTTTTATAGCTAAAAATCTGATTTGATTATTTAGTAATGGAGAATTTTACCAGAATGTGATTAAAAACCAGTCTTTTGTCACCA
AAAGATCTTTTATGTCACCAAAATAAAATCCTTGACGGATACAACACTACTAGGTGTATCATTAAAATATATTCTTAATAGAATAAACACCTAG
AATCAGAACATTGGTTGAAAACGTAGGAGCTTTACAGGGGCAGTAAATTTGGAGCTAGTCACATTTCAAATTTGCCATCAGTGTTTCTTTGTTG
AATCCAGAAGATAGAGCAAATTGAGCTGATAGTGTAAGTTAGCAACATAGTTTTATAAAAGTATTTGAGGGACTGGCCAGAAATATACTCCTGA
CTGTACATGTTTCATAGGAAGTTCTAGAAACAACAAAGCACTTTCTATATGCTTTTAGGTGGAATCTCCAACTGATAAATCAGGAAACTTCTTC
TGCTCACACCACTGGGAAGGGGACAGTCTGAATACCCAACTGTTCACGCAGACCTCGCCTGTGGCAGATGCACCGGTAAGTACGTGTGCACCAG
CCCCCAAGTTGTTATGTGACACTGGTGCCTCATGAAATCCTGTGTGTGGCATAGGGCTGCTGCGGTTTCAGCACTCCCTCTTTAACACATCTTA
TTTCTAAATGTTCTCCTGCACATGATTTTTAAAAAATGATTATTAATGTAGAGTATAAAGTTGAGATTTTTTCATGTATTAGAAACATTGCAGA
CTTTTTCATTATTTGTTTTCTCATTATTTGAGTGCCTTGGCCATTTAAAAAAATCTACATGGATATAAAAACTATGTTCATTTATATATTTTCC
TTTATTTGTCTTCTAAATAATATTGTCTTCTTGTTAAGTTGTCTCTTGTTTGGTTTGTGTAGCATCTCATATGGTATCAACCACGTTGTCTATA
GTAGATGCCCAGTATTGTACCCAATTGCTTTGGGTTACTACCACCATTTTTAAAGTTTATAATTTGTGAAGGGTTGTCCTTATGTCTTAAGGAA
AATCAGAGCTAATAACAGCTGCTAAGATTTATTTAGTAGGTACTAAGTGGTAAGTACTGCTCTAGGGGCTTTCGATGAATTGATTCAGTTAATT
CTCACAATTATCCTGTAAGGTTGGTATTATTTTTATTAATTTTTATTTTTTTTGAGACGGAGTTTCGCTCTTGTTGCCCAGACTGGAGTGCAAT
GGCACGATTTCGGCTCACTGCAACCTCTGCCTCCCGGGTTCAAGCGATTCTCCTGCCTCAGTCTCCCGAGAAGCTGGGATTACAGGCACCCGCC
ACCACGCCCAGCTAATTTTGAATTTTTAGTAGAGACGGGGTTTCTCCATGTTGGTCAGGCTGGTCTTGAACTCCTGACCTCAGGTGATCTACCC
GCCTCGGCCTCCCAAAGTGCTAGGATTACAGGCGTGAGCCACCGCGCCCGGCCAGGTGGGTATTATTATCATTTCTATTATGTCAACTGGAAAA
CAGAGGCACAAAGAGGCTTAGTCATTTGCTCAAGATTGTATACCTAATAAGGGATGGTGCTGGGTTGGAACTCAGAAGGTCTTGTTCTAAATTC
CCTACTTTTCACCATTTTTTGCCAATAATTTATAGCTCTAAAATATTGATAGTTCTGTACCAATATATGAAATAAAGTGTTCTCTCCCCAGAAC
TTAGTAAATCTTCTATTTTGAAAGGCATGTATTTATTAATTCATTTAGCAGATGTTTTTTGAGTGCCTAACACATGCCTAGAACTCTGGGATCC
CAAAGGAGCTTTGTGTTGTGGTGATGTTTGCCACAATGGGATTAAGGTTCAGACATAGTTGATTTCTCCTTTTAGCTCATAGTGGGGAGAATTT
CTGGCAGATGACTGCATTTTTTTATGCTAATGTGTATCATCTCATAGTAATCTACTCTCCACAGTGTTAGAAAAATGTGGCTCTCATATTATCC
AACCTCATTTAAATTTCAAATTATTCTCTTCCCTTTAGAATATTCTTACAAGCTCCGTTTTAATGGCACCAGCATCAGAAGATGAAGACAATGT
TCTCAAAGCATTTACAGTACCTAAAAACAGGTCCCTGGCGAGCCCCTTGCAGGTAATTACTATTCCAACATTGGTATTTTAAAATTCATTCACT
GAAAAACTGTCATCTTTGGTGGTGGTGGTGGTGGTGGTGATGGTAGTGCTGGTGGTCTGTTTTTCGTTTTCAACATTTTAGACATAGGGGAGTA
ATGCTTTTATACAATTCTTGATGTCTAGAAAAATCCAATAATTCCTTTTTGCTACTCATATGAGACATGCTAGAAATTTTTATCAGTGCAGATT
CCCCAAAGCATGATGAAATGAAACATTTCTTACTGTGACTGAGACTAAGATGTATTACACGTGTGTCACTATCTTCTTCTGCCCTCAAATGTTT
TATAAAAATATATCAATATAGTTTACTTTATGTTCCAAATTTTTTATTTTTTATGCTGTATCCCTAGGCAACCAAAGCTCAGAGACTGTTCCAA
TTTTAAATGAAGTCTGAAATTAGTCAGACAGAAAATAATTGCAATGTATTTGACTGTCAGAATAAGAAAGTCAACTGTCAATGTTGTGAGGCCG
ATCTGAAGTTGACCATCTGCTGTCAGGTTTTAATATCAAAAGAGATTTCCCTTGTAACCCTAAAGTGGGTGTGTGTTGATGAAATATAACCAAT
TCCAAATTAATCCTGAGCCAAATGTAAATAAGAATGCAAATTTTGCTTCCCTAAATTATTTAAAGCAGTTTCTGACATTAAATAGCCCAATACC
CAGCCACCTGCAGAGCCCTCTCCTCCATCCCCCTGTGCCATGCAGAGGGAAACTTCATAACCCTAGAGGAAGTGAAGAGCCTGGATTCAGAAGC
TGAAGGTCATATTTCTTGCTGTAAGTTAAATGGCACGATCATAGGACAGCAGCCTTTGAGTCTGCCCATTACCTTAAAGGGATTTCCCATTCTT
GTGATAGTTTCTCCTTCCAAATTGTGAGAATCTGAAAACTTGCCCGGTAAGTATTTGTGATGCTTAGTTTTTCAGGGTTTTTTGTTGATGTTGC
TTTGTTTTTTGAAACAGGGTCTTACCCGGTGGCCCAGGCTGGAGTGTAGTGGGGTGATCTCGGCTCACTGCAACCTCCGCCTCCCAGATTCAAG
TGATTCTTATGCCTCAGCCTCCCAATTAGCTGGAATTACAGGTGTGAGCCACCGGCAACCCAGCCGTTTTTTTGTATTTTTGGATAGAGACAAG
GTTTTGCCATGTTGGCCAGGCAGGTCTCGAATTCCTGGTTTCAAGTGATCTGCCCACCTCGGCCTCCTACAGTGCTGGGATTACAGGTGTGAGC
TACCGCACCTAGCCAGTGCGTAGTATTATTGTAAAATAAGGATTGCAAGCACATTTTGTTTAATTAAATCAGATTGCTTCCTTCCTGGCCTCTC
TCTGACAGCAATTGTTTTGGTAAAATGGCTGTGGACTGGAATGTGAGACAGGGAGTCAGTATTTATGGGGAATCTGGACAAGTTCCTGCGTCAG
TGAGGCTGGAGTTGGCCCCGTGTGGAGAAGACTCAGAATACCACAAAGAGGCCAGACGCAGTGGCTCATGCCTGTAATCCCAGCACTTTGGGAG
GCCAAGGCGGGTGGATAACTTGAGGTCAGGAGTTTGAGACCAGCCTGGCCAACACGGTGAAACCCCATCTCTACTAATCCCAGCTACTCGGGAG
GCTGACACAGGAGGATCGCTTGAACCCAGGAGGCAGAGGCTGCAGTGAGCCGAGATCATGACACTGCACTCCAGCCTGGGTGACAGAATGAGAC
TCCATCTCAAAAAAAAGAATATCACAAATAATTGGGAGATGAGGAAGAGAATGAACTCCACCCAGACATAAGAGGAACCCAGAAAGGGAGTTAC
AAAGATTTCAAGGCAAAGGGCAGCCATTTCAAAATTTTCCTGAAAGCTGTTCTTAGGTCCAGAAGAAAAGCTTTTGCTTTTTTTTTTTTTTTTA
ATTATTCCCACCTAAGTTACTTGGCTCTTGGTTTCTAGAGAACAGAATTCAACTACTGCTATTCGTGTTTTAATTAAGTTAGATTAAGTTTTGC
ATATATTTTCTAACCTAGTCTGGGGCCAACTTGTATTTGTAGTAGTACAGAAAAAGTCTGTTCTAAATTTCAAAAAGCCAACACATCAATATAC
TTGGGGACAATTACCATTACATAAGTTGAAACCCATAGTTCAGTATTGAGCTTTATCAGTATATTTGTATTTTATTTAATGGAAATATTGGTTA
TCTTTGAAACTCAAAATTAAGTTATAGCTCTATTTTATTAATCTGTATTCTCCTGTCTATCAGTGAGTCATATTGGCACTTTATGAATTCTAAC
ATCTGTACACTGAGATTAAGTTGTGTATGGTGAAGGTAATATTTGTCAATTCTATCAAACTGAATAATTGAGACTTGAAAGTTAATTTTAGAGT
CAATACTCCTGTCTAAACCATATAGTTTACTGGTTTACACTGACCAGATATATTTTAATAATCTGCTATAAAGCCAGGCTCAGTGGCTCATGCC
TGTAATCACAATACTTTGGGAAGCCAAGACTGGAGGATAGCTTGAGACCAGGAATTTGAGACCAGCCTGGGCAACATAGTGAGACTCAGTCTCT
ATTAAAAAATAATTAGCTGGGCTGGGCGTGGTGGCTCACGCCTGTAATCCCAGCACTTTTGGAGGCCGAGGTGGGCGGATCACGAGGTCAGGAG
ATCGAGACCATCCTGGCTAACACGGTGAAACCCCGTCTCTACTAAAAATACAAAAAAATTAGCCGGGCGTAGTGGCGGGCACCTGTAGTCCCAG
CTACTTGGGAGGCTGAGGCAGGAGAATGGCATGAACCCAGGAGGCGGAGCTTGCAGTGAGCTGAGATAGCGCCACTACACTCCGGCCTGGGTGA
CTGAGCGAGACTCCATCTCAAAAAAAAAAAAAAAAAAAAAAATAATAATAATAATAATAATAATTAGCTGGGTGTAGTGGTACATACCTGTAAT
CCTAGCTACTTGGGAGGCTAGGGCAGGAGGATCCCTTAAGCCCAGGAGTTCAAGGTTACAGTGAGCTATGATTATACCCCTGCACTCTAGCCTG
GGAGACAAGACCAAGACCTTGTCTCTAAAAAAAATTTTTTAAGGCCAGCCGTGGTGGCTCACACCTGTAATCCCAGCACTTTGGGAGGCAGAGG
CGGGTGGATCACAAGGTCAGGAGTTTGAGACCAACCTGGCCAACATGGTGAAACTCTGTCTTTACTAAAAATACAAAAAATAGCCAGGCGTGGT
GGTGCGCACATGTAATCCCAGCTACTTGGGAGGCTGCGGCAGGAGAATTGCTTGAACCCAGGAGGTGAAAGTTGCAGTGAGCCCAGATCGTGCC
ATTGAACTCCAGCCTAGGCAATAGAGTGAGACTCTATCTCAAACAAACAAACAAAAATTTAATCTGCTATAATAGATACTAATTTATTTTAAGA
AATACTTAGTGAATGTTGTTCTACATACTTGCCAATTGGGAGGAAAAAAGAAATATTCACTAAATGAATGAATGTTATACACCAAGTATCTCCT
TCATTTTATGTCATGGAGTTCTAGCTTCTGATATCACAGAAATATGTGATTTCTAAAATGTTTCAGACTATTGTGATAAGTATTGGAAAATGCA
TCAAGTGACTCTAAATATTTAAACATACATCACCTATATTTCATATAATCCTGATTCAATAATATTTCTATAGATTTTTTATGCAATTTGGTAA
TTTTGTTTGTTTGAAACACAGTGCTATTTCATATATTTCTATTGATAATCCAATTTGACCTAATTAGGCATATTTCTCACACATGGTATTATTG
AACATATTGCACATTTATTAAATGATATGTATTTTACTTAAGAGTACTTTATATAATCTATAATTTTTGTAATTTGTACTTATAATAAATCAAA
TATTAAGTGGGATTTTAGCCAAACCATCTAGACATTTTTGTCATAATGTTTTTATTAAAACAGCAAAATAGGAGTTTCAAAATATTTTAAAATT
TGAATACTTCTAAAATGTAAAATGTTTGCAACTGGTGTAATGCAGTTGATTGTCTATCCCTCTAAACTGTCTAAATAACTTAGGTTCAAGAACA
GTTTATTGAAGAACATAGGAACTGCGCAATTCTGTGTTTTATCATTTCAGTTTTAATCACCCCGGCTTGATTATGGCAACAACATTTTAGACAA
GCAATTATTACTATTTTTGAGGCCCATATACTGGGCCATTATGGTTTTGCTTCTAACGTACATATAAATGCACATGCAATATATATAAATTATG
TTTGCTGTTTTAAAATTAGAAATGCCGAGAAGCTATGTGTTTGACATTTTATTATAAATTTTGTACACACATAGATGTATGGAAAATATACTAT
GTTCTAGAGTCAACAATTGTTATCATTTTGCCATATTTACCTCATCTCTTTTTTTTTTTTTTTGAGATGAAATCTCATTCTGTTGCCCAGGCTG
GGGTGCAGTGACGCAATCTCAGCTCACTGTAACCTCTGCCTCCCAGGTTCAAGCAATTCTCCTGCCTCAGTCCCCTGAGTAGCTGGGACTACAG
ATATCTGCCTGGCTAATTTTTGTATTTTTAGTAGAGATAGGGTTTCTCCATGTTGGCTAGGCTGGTCTCGAACTGCTGACCTCAAGTGATCCAC
CTGCCTCGGCCTCCCAAAGTGCTGGGATTATAGATGTGAGCCACCACGCCTGGGCTACCTCATCTCTTTTTTATGAAGTTTTTATTTTTATTTT
TTAATTTTTTTTTTAATTTTTTTTTTTTTTTGAGACAGCGTCTTGTTCTGTCACCCAGGCTGGAGTGCAGTGGCATGATCTCGGCTCACTGCAA
CCTCCACCTCTTGGGTTCAAGTGATTCTTCTGCCTTAGCCTCTCGAGTAGCTGGGACTACAGGCGCTTGTCACCATGCTAGCTAATTTTTGGTA
TTTTTAGTAGGGACGGGGTTTCACCGTGTTAGCCAGGATGGTCTCAATCTCCTGACCTCATGATCGGCCCGCCTTGGCCTACCAAAGTGCTGGG
ATTACAGACATGAGCCACTGCACCCGGCCAGTTTTTTATTTTTTATTTTTTTGAGACAAGGCCTTGCTCTGTCACCCAGGCTGGAGTGCAGTGG
CAAGATCATGGCTCACTGCAGCCTACCTCCTGGACTCAAGCAATCCTCCTCCCTCAGCCTCCTGAGTAGCTGGGACTATAGGCACATGCCACCA
TGCCCAGCTAGTTTTTTTTTTTTTTTTTAGAGATGGGGTCTCCCTGTGTTGCCCAGCCTGGCCTCAAACTCCTGGGCTCAAGCAATCCTTCTGC
CTTGACCTCCCAAAGTGCTGGGATTACAGGCAGGAGCCACCCTGCCCAGCCTGAAGTATTTTAAAGTAAATTCCAACTAATATAACATTTTATT
CTCATGTACTTCCAGATGTGTCTCTAAAAATAGACATTTTTATATATAACCTAATACCATCATTACATATTTAACAATAATTCTTTAATCTTCT
CATTCTCAGGTATATTTAAATTTTCCCATTAATCCAAAAAAGAATCAAAATTGAAACAATTTCACACATTGCATTTGGTTAAGCCTCTTAAATC
TCTTTTACTCTCTAGTCCAGTGCTGTCTAATAGAACTTCTACAGTGATGTGAAGGAAATATCTAATATGCTGCTATCCACTATGATAGCCAGTG
GCTTTTGAGCACTTGAAATATGGCAAGTGCTGCTGAGGAGCAGAATTTTCAATTTGAATTAATTTAAGTTGCTTTAATTTAATAGCCACATTTC
CTTACTCTCTTACTGGAATCAGCCATTTCCCCATGGTGCTCTGAATCCTCTTAGTGAGAAATGGTACATAGACACCAAAATCAGGGTGCTAAGT
CTGTCCATTGTTATGTGGTTTGTCATTGAGATACAAATAATTTTTTTTGTTTTGTTTTGAGACAGAGTCTCACTCTGTCACCCAGGCTGCAGTG
CAGTGGTACAATCTTGGCTTACTGCAACCTCCACCTCCTTGGTCCAAGCTATTCTCCTGCCTCAGTTTCCCAAGTAGTTGGGATTACAGGCACC
CACCACCACACTCAGCTAATTTTCGTATTTTTAGTAGAGACATGGTTTCACCATGTTGGCCAGGCCAGGCTCAAACTCCCAACCTCAGGTGATC
TGCCTGTCTCAGCCTCCCAAAGTGCTAGGATTACAGGCGTGAGCTGCCACACCTAGCCAAGACTCAGTGAATTTTAATAATCCAGAGCTCTCCA
TGTATCCCCTAATCTATAATCTACAGTGCTTAAATAGAGGCTTTTATAAGCCATTATGTACACTCATGTTATGTTTGTTTTTTTAATTACTTAG
TCTTTTTCCAAAAGGAATTTGCAACAGCTACATAATCTTATAATTGTTATGTTTCTTTTATATTTTCACAGTTGTTTTAATTTTAAATTGTAAA
AGCAAACAGGATATTCTTATAATAAAAATGTTCTGAGATGTTTTTCATATTTGTGTCTTTCATGTGGCTCAGAATATATGAGATTTTCATGTCA
TATCATTGTCAGTATTCAGCCTTTTCTCCACATTGTGTGGGGTAGAGGATAGAGTCTAGAAGCTTATAAAATGGTCACAAATGAGGAAGTAGAG
AAAACATTCAGCCTAGAAAGCGTGCATTTGTGGTATGAAAGGCACTGTGGAATGCTGCTGGATTCTGCCCTTCAGTAGTTACCCGAGAGAGACC
GAGGCAGACGCATAGCTCCCTCCTCAGCAAGGAGAAAAGAGTTGCCTGCTTTCCTCAGCGTGCTTTCCATTTCCTTTGTTTTGAAAAAAGCACA
TTATGCTAATCAACTTTCCTTAAGGTAGACATTTTTATTGGTAGATCAAAAACCGTGCTTTAAGTTATAAGCCTTCTGAACTTCTGTATTTTTC
TCTTAAATTATAATTTCCTTTTATTCCTATTTAAAACCCAAGTGTAAAAGATGTGTTCATAGGCTTTCTACTCACTCACTTGCCTCTTTTTCGT
AACCACAGAATGACAGCCTTAAATTTTAATTTCCTGACTTGGCTTGCCCATGGCCTGACTTTTTTAAAACAAGGAGTTTAAGTTAAAGAAATTT
CTCCAGCCATTATTATTTAAACACATTTACACAGTATCAAAGAATTTAAAAATGTTGCTAGCAGTTGATACTCCCCTCAGCTTTCGGTTCAGCG
TGTTTTAGATTGCTCCTATTTTGTTATTTGTCTATGAACTTTTATATTTTTCAAAAAAATAAGGCAAGCCCTTTTTGCCTGATAGGCACTGCTG
AGCAGTATAATAAACAAGCATGGGGGCAACAGAACCTGGGTTCCAGGCTAGACTCCACTGTTTTCAAACGGCATGACTTTAATTCATCAAGTGA
ACACAATTAAGCAAAGTATAAGTTTGGGACCATGGAATATCCTTCACTCATCACACAGGTTACTGTGAAGATCAAATGGATATAATATTGCTTT
CAAGATGGCGGCACACTTGCCACTATACACAGGTGATATTATTAGGCAGAGTCGCCACATAGTCCCTGTCCTTGAAATGCAGGCAAGGAGCCCT
CACCAAAGGCCAGGGTGGTTTTTTGTTTTTCGGGTTTTTGTTTGTTTTTTTCATTGTGGTATATTATTTCCTCTGGCATGCTATTCCCTGTCCC
ACTCTTACAGAACTGTGTCAGTGGGTCACCAAAAGAGAGAGCTGATTCAACTGATTCCTTGAAATAAAGGTAGTCAGACCTCGCGGTATGATGG
AGATGAAGCAACTGATTTACTGGTTTTCTCTATTTTTCTGCTGTTCGTGTTGTCATTTAAACAGACATGGAAACCGTCTGCACACGACAGAGAT
CTTTCCGAGCCTTTTTCATCTAGTGTGCTGTGCTGACCCGCTCCCCTGGATGCCTGCACACTCATTTGGTTCTCCCTTTTGGTCACTTTTTTCC
ACTTACCCTGGCAGAAATTCACGTGGCTGTCAGTAGTAATTCAGTGACCTGCTTTCAAAAAATGTTATAAATTAAGCAGCTTTTCTGTGTATAG
CTTTTGGGTTGAACTGAGAAGGGCTAAGTGAAGGGAGAAGCTATGTTGAGCTCAGAGTCAGAATGCCCTGCCCCAGACATTTTGAGATGTGAAT
TTTTCATTTGTTTTGTTTGGTGACACTTAGAGAGAATAAACATTCTGAGATAAAAATCTTCCTTGGGCAGGCCAGCAAAGATAGAATTGCTTTA
GAGCAATTTGGCACTTGGTCTTAATCTTAATAGTTTGGAACTCATATGTGACTAAGGAAAAGGATATTGCAAAAAGTTGTGCCACCCTAGGCTA
TATGATATAAACTTAGAAAGTCTATCAACGTGAAAGGTGAAGAGACCTCTAGAGACATCGGGGACAAACTCCAAATTTGGGAAAGGTCCATCCA
CATTTGCAATTGTTAGTTAGGCTTGGGAAGTGGAGTCATTGGTGGATGAGGCAACTTCCAATCACCATCTCGATTTTTATTTCCCTAACTTTTA
AGATTATTTTCTATAGAAAATCAGTAGTGCTGAAGTTGGAGTCATTTAATTGATTTGTGCCAACCTAGGGAAGCATTTACTCTCGAGAAAAGTT
TGGTAACCATTAATCTTACCTTAGCAACATTTCAGTTTAAGCTTATCTCTGACTTTCCAAAGACAACTAAAGATTTCTACAGTCTCTCTTATAA
CTGATCCATTTTTTCAAAAGATGTCCCTTTGGGAAGGAATTCTCATTTTTAGAATTTTAGTCCAGAGGAAGTATTTTTTTTTCAGATAGGTGAA
ATAGAATAAGCAGAAAATTCTTATTTTTTACTGTTTTATATACCATTGATGCCTGCTTATTTTAAAACAAAACACAATAAAAACATGATACCAT
CTGATGAGTTGCTTTGGTTTTACTTTTTTTTTTTTTTTTTTTTTTTTTTTTTTTTTAGTTTATACAAGTTTCTTATTAGGTCAGCAAATTGTAA
AACACATAATTTGCCAAGGCTTTTGAAGCCAGCAGATACTGGGTGAAGTTGCTGTATGAATATAATAATAAGAACCGTTTTTGTTCAGCTTGAT
GAAAAAACTATTCATGTTCTAAATGAAAATGTGAAGATCATCTAATTCTGACAGAAAACCACCTGTGGACATGGTCTTGTTTCTTTTTATTTCT
TCTGTTTTCTACAAAAGATGGCAATATTTATTTAAAGTGCTTTAACAGGATATCCCAGTTAAAGCTTTGACAGACTGTTTATAGTTCACTTCTG
CCCAGGGGCAATTAAAGGCACATTACAGGATAAGCGAGATGACTCCCCGGCCACAGGAGGAGGATGTGTGGGTAGGATTATTAATGTTGAAATG
AATTATGATAGAGTGACATGTCTGCAAGAGCACCATGGGTTAGTAACTTAACAAGAATTCATATGCAGAAACCCATAATTCTCAGCAGTTCTTT
AGGGTAAATGAAACTAAAGTTTAGGGAGAGAGAACAAGGAGAATTTGCAAAGATATGTATGACAGTATTTAAAAATGTTCTTCCAGCACAAAGA
CATTAATCTTCCTTTGTCAAAATAATTCTTTCTGCATGAAAAGTTGGCATGGGGCTCCCAGGCTTCAGGTAAGATGGGAGAAAGGGAGCGTTTC
TTTTTTTTTGTACCAAAGTACTCAATCTTCTGATCTTCCTTGACTTTCACGACATAGGCATATGTCTACTCTGACCAGTTTGTTTCATTTATTG
TGGTAAGATGTGCTTGTCTTGTGCTTTCCTGGTGCAGAAAAGGAGCTGCCTCGCTCATGCTTGGTGGCCTTTTTGTTCCTACACACAGTGGTTC
TCGTACATGGTGGCTACCACATCCTGGTGTTTGTCTATACTGGGAGAATCAGGCGTTCCAAGTCATCTTGGCTGCCCTAGCTCTGAGCTTCAGG
GGATATATGACTCAGAAATGCTATGCTTTCTGGAATTTGGATATTTCATTTTTATTTGTTTCTTGGTAAATTCTCTTTTGACTTAGGAGAAGCT
AACTATTTGGAAAGGTCTCTCAGAACTCTAATTACAAATATATGGTACCTTCTTTATATTGATCTCATTTTTAAATACCTAGTCCTGGAGGAAG
AGCTAAATATGAATACAGTGATGAGCTGTGTAGGGGATCTCACAGGAACACAGTCAGATTTCAAGTTGAAAATAGATTCAGTTGATAATACTTA
GTACTAGATTCTCCTGAATCGACTTTGGGAATTCTGAGTCAACCTAAGGACTATATTACAAAGGATCAGACCTTGACTTTTAGGCCAGGTGTAG
TGGCTCATGCCTGTAATCCCAGCACTTTAGGAAGCCAAGGCAAGTGGATCACTTCAGTCCAGGAGGTTGAGACCAGCCTGTGCAACACAGCGAG
ACCCTGTCTCTACCAAAAATACAAAAATTAGCCAGGTGTAGTGTAGCACCTGCCTGTAGTCTGAGCTACTGAGGAGGCTGAGGTTGGAGAATCA
ACTTTAACCCCGGATATGGAGGCTGCAGTGAGCTGTGATCATGCCAGCCTGGGCAAGGAGCAAGACCCTCTCTCAAAAACGAAAACAAAAAAGA
CACAGAACTTGACTTTTAACAGGAGGCATAGTAATAGCAAGCATAAGATTGTTTTAACATAGCATTTAAGATAATTTACGCATTTAGAAAATTA
ATTCTGGCTGGTTGTAGTAGCTTATCCTTAATCCTAACCCTATGAGAGGCCAAGGCAGGAAGATCACCTGAGGCCAGGAGTTTGAGACCAGCCT
AGGCAACATAGCGAGACCCCCGCTCTCTACAAAAAATTTTTTTTAAAAAACATAGCTGGGCATGGTGACACACTCCTGTAGTCCCAGCTACTCA
GGAGGCTGAGGCGGGAGGCTTGCTTGAGGCCAGGAGTTTGAGGGAGCTGTGATCACGCCCCTGCACTCCAGCCTGGGCAACAGAATGAGGCCCT
ATATCTAAAAAAAAGAAAAATGGACAAGAAAATTCTATATTGATTGAAGCCCAGCTAAATAGAAAAAAAAAAATTCTAATAGGCCATTTTAAAG
ACTTTGGAAAAATTCTTAGCTAGAGTAAAACAGAAGCAATCCTTTCTTTCCTGGTTCTCACATCTCCATTCTCACCTTCATTCCCCTTCCCCTC
CATTTGTTTCAAGTATTTTTCTTTTCTATGTGTGTCTTTATGTAAATTTAAATTTACCTACAAGATATTATGATATTAAGATATTTCATTCTGC
TTCCTGTTTTGTCACTTGTTCTTTAAGATCCAAGTCCTTGGCTCCATGTGCATCTAATCCTTGCCATAAGCCCTGCATCGTACTTCATATTCAC
CATATTGAACTATTCACCCTCAAGTAGTCACTATTAAATGTCACACTGTTGTAGCGACTACAAATGCAAGCTCAGTAACTGAAAAGATAACATT
AAATTTTGTTTTTTAATTGGTTTCCTGTGTCAATTTCATGTTTCGGGAATTTGTATGGACAGATAGACACCAGAGAGATATGCGTATTTCACCT
GCATGCTTGAGGCTAGAGAAACATGATTTATTCAGAACTATTTCAAATACTGTAACTAACCTGGTAATTTTGTTTAGGAAAACATTAAATTCGG
AATAGAAGGCAATTCAGAATACCTGTCTGTTATCTTTGTCTTGTCATTTTTGAGATACTCAGTTCCTGGAAAATATTATGATAATCCATCTTGC
TATGTAGTATTTCAAGTTATATGTTGAGCCTCTGAGCTTGCAGAGTGAACATTTCTATTCATAGACAGTGCTGTTGACATCAGTGGGGAGATGG
CACAAGCTTTGTGCCCTAGGAAAGTTTAAAAGCTAGAACATTACACTGCAAACCCTATGTCAGGAAACTTTGTTGCAGCAACTAGGCAAGGAAA
CCCTACCACTGTACCCATACCTTGTTTTAGTAGAATTATGTTTCTTTGCTAAGAAGTCTGGTGACTCTCACACCCCATCTCAGCACTTTCCTGG
ATTCCAAGGCCCTGGTGCCCTGAGCACACAACTTCCCAGGTTCCTTTTATTGTTTCTGATAAGAACTCGAATTAGAGGCAGCGTCTGGTACTCA
CCAGGGGTGTGGTCCCATGGGTGACATTCTTGCTCCATCATTTCTTACCCAGGTAACCTTGAGTATGGCGCCTAAATCCTCTCTTCCTTTCAAA
TTGCCTGGTCTGTAAAATGGATGCAGTGAATGGTAGCTATGCCACAGGTGTTTTGTGTGAGAATTAAATGAGAGGAGTTATATCAATAACACTT
AGAGGCATGCGGTGCCTATAGTGAGGGCTTGTTTTATGTTGTCGTTTAACCCTTTCTTTCTCAGCCCTCCCCCACTCCACATCCCCTCCCACTG
GCAATGTACAAATTGCAGCTTCCTGCCTCTTTCCTGCTATCTCGCTCCCTGGATGACATCCCTGCGTTTCCACCTCTCTCTCCCCTAATCTTAG
CTGTTTTCACCCTTTCCCCACAGCATTGCCTTGACAGTTCCTCAGTGTCCCATCCCCTCTTCCCATGGACAACACCACAAACCTGCGCACACGG
CCTGTTATTATTCCTCCTGTTTCACCTGTGTCCATTCTGTCTCATAAAGGACACTGTAGGCTCTGAACAGCTAAGGCCTTTCTTATTCTTTTGT
CCCATGGAAAATAATCCCTCCAGATAGCAATACCAGGAATAGTGCCTTTTCTAACCCACTGGTTTCCTTTTGAGCTTACAGCCACCAAATCTTA
TATCTGTTTGTTTCTGTTCCCCTCTCTGCATTGACTGCTAGAGAGATCAGAGTCAAGCAGGAGGTTACATCTAGCTGGGGCACTGTGCTTTATG
CTGGTGTTTCGTGTGCAAATCTCATACCATCTTCATGTTACAGTTGTTCCTTAGTGTCCATGGGGGATTTGTTTCAGAACCCGCAAGGACACCA
AAGTCCAGGGATGCTCAAGTCCCTGATATAAAATAGCATTGTGTTTGCATATAATCTACACACATCTTACCATATAATTTTTTTTATTATTTGT
ATACATTCGTGGGGTACAAGTGTAATTTTGTTACATGGATGTATTGCATAGTGGTCAAAGTCGAGGCTGTGAGTGTATTTATCGTTGGAATAAT
GTACATTGTACCCGTTCATTCCTGTATACTTTAAGTCATCTCTAGATTACTTATAATAGCTAATACAATGTAAATGCTATGGAAATAGTTGTTA
TATTGTTAGGGGATAATGACAAGGAAAACACCTATACATGTTCAATACACAGCAATTATTTTTTTCTGAATATTTTTGATCTGCCATTGATTGA
ATCTATGGATGTGGAACCCACAGATATGGGACCCACAGATGCAGAGGGCCAACTGTGTTTATTTTTTCCAAATTTGTTTCCCTGTTATGACTCT
TCTTTATACACATTTTTAAACTCTGTTGTCTTGTATTTCTTTTCTAATCATCTTACGTCCTTTTGGAAACCAAGTGGGGCATAAATAGTACATG
TTTGCATTTGAGGCAACACAGAGGCATCGCAGGAGCTGGAGACAGCAAAGTTCAAAGCCAGTCCACCACTTACCTACAGAGTGGCCTTGGGCAA
GTTACCTCCATTTTCTGTCTCCATTCTCGAGCCTGTCTAGAGAGAGAATGAACCTGCCTCCCGAGGTGATGGTGCTGTTTTAATGAAATAATGC
ATGTGAAATGTGAAGTGCCACTGTCCCTGGCATGGAGATGCTGTCCTTAGATATTAGGTCCATCTTGTATCTTCTAGTGTATCTTTAAAAATTA
AACTATACTGGCTTCATGCCTGTAATCTCAGCACTTTGGGAGGCCAAGACCGGCAGATCACTTGAGTCCAGGAGTTCAAGACCAACCTGGGCAA
CATGGTGAAACCCTGTCTCTACAGAAAATACAAAAATTAGTGGGGCTTGGTGGCACGTGCCTGTAGTCCCAGCTGCTTGGGTAGCTGAGGTAGG
AGGATCACTTGAGCCCAGGAGGCTGAGGCTACAGTGAGCTGTGATTGCACCAATACCGATCAGCCTGGTATGACAGAGCATGACTCTGTCTCAA
AAAAAAAAGGAAAAAAATTAAACTATACTGATAAATATTTCTAAAGTAATTGATGAAAACATATATATGACACTTAGCAGGTATTGATCAATTC
CAGTGCGTGCATGAGCTGCTTTGCACTGTGGCTACGGCACTCAGCAGTTTGCTTAGTGAGTAGATGGCCATAGCTCACTTTAGGTATCTGGAGA
ATAATGCTCATTCCTGTCTCCAGCTTTGTAAAATTGCCTAATAGACGGAAGGCATTAAAATATGAAGATGGGATTATATCTTATGAAGCTGCTT
GGAATGGGTTTTCCCTCTTAACAAATAATTTCTGATCATCCATATCCCTTAAGAACACCGGATTTCTGGGGGCCAGGGAGGTAAGATTCTATCT
GACAAAGCCTTCCTGGTGTCAACCACTTGCCATCTGTTGGTCAGTGCTGGCCCTGCTGGGTCTATAGAATGAGCTTCTTTGTCTGACGCTCCTG
TTGCCATCCCTTTCTCCATCAGCCTTGTAGCAGTACCTGGGAACCTGCATCCTGTGGAAAGATGGAGGAGCAGATGACATCTTCCAGTCAAGCT
CGTAAATACGTGAATGCATTCTCAGCCCGGACGCTGGTCATGTGAGACATTTCCAGAAAAGCATTATGGTTTTCAGAACACTTCAAGTTGACTT
GGGATATATCATTCCTCAACATGAAACTTTTCATGAATGGGAGAAGAACCTATTTTTGTTGTGGTACAACAGTTGAGAGCAGCACCAAGTGCAT
TTAGTTGAATGAAGTCTTCTTGGATTTCACCCAACTAAAAGGATTTTTAAAAATAAATAACAGTCTTACCTAAATTATTAGGTAATGAATTGTA
GCCAGTTGTTAATATCTTAATGCAGATTTTTTTAAAAAAAAACATAAAATGATTTATCTGTATTTTAAAGGATCCAACAGATCAGTATTTTTTC
CTGTGATGGGTTTTTTGAAATTTGACACATTAAAAGGTACTCCAGTATTTCACTTTTCTCGATCACTAAACATATGCATATATTTTTAAAAATC
AGTAAAAGCATTACTCTAAGTGTAGACTTAATACCATGTGACATTTAATCCAGATTGTAAATGCTCATTTATGGTTAATGACATTGAAGGTACA
TTTATTGTACCAAACCATTTTATGAGTTTTCTGTTAGCTTGCTTTAAAAATTATTACTGTAAGAAATAGTTTTATAAAAAATTATATTTTTATT
CAGTAATTTAATTTTGTAAATGCCAAATGAAAAACGTTTTTTGCTGCTATGGTCTTAGCCTGTAGACATGCTGCTAGTATCAGAGGGGCAGTAG
AGCTTGGACAGAAAGAAAAGAAACTTGGTGTTAGGTAATTGACTATGCACTAGTATTTCAGACTTTTTAATTTTATATATATATACATTTTTTT
TCCTTCTGCAATACATTTGAAAACTTGTTTGGGAGACTCTGCATTTTTTATTGTGGTTTTTTTGTTATTGTTGGTTTATACAAGCATGCGTTGC
ACTTCTTTTTTGGGAGATGTGTGTTGTTGATGTTCTATGTTTTGTTTTGAGTGTAGCCTGACTGTTTTATAATTTGGGAGTTCTGCATTTGATC
CGCATCCCCTGTGGTTTCTAAGTGTATGGTCTCAGAACTGTTGCATGGATCCTGTGTTTGCAACTGGGGAGACAGAAACTGTGGTTGATAGCCA
GTCACTGCCTTAAGAACATTTGATGCAAGATGGCCAGCACTGAACTTTTGAGATATGACGGTGTACTTACTGCCTTGTAGCAAAATAAAGATGT
GCCCTTATTTTACCTACTTTTGTTTTCTCTCTTATTTGTCACTATGGACAACAGTTACACTTTAAAGAAAATGGAGCAGACTCAAAGCACTTTC
CTCTAATAATTGCACACCATATGCACTTCGTGTGAAGAAGCCAGGAATCATTACAATGGAGAAGAAAGAACAAGCAAACTGAGTTAAATAGTTC
ACAAACAATCACATGGACAATGGGGAGGAATCTTCCAGAATGCATCAGTCAGGGTCTAGCGGCATTTGAGTGCGGATGCTGTAAGGGGAATTCG
TTCACAAATGTACTGATACCACAGATGTGGGCAGGGGCTAGGAGAGCAACAGGGTGCAGGCCTGAAGGCACCTGGAAGGCCGCCTGGAGGGGGA
CAGTCAGGAAAGCAGGAGCCCTTGAGAGGAGCACTAGCCTTTGATGAGACCACTGCCATCGAAGGAGACCTCACAGGGAGGGAGCCAAGGGTAA
ACCCTAACTTTGTTTCCCCATTTCTCCTATCTCTGCCTAGGGGTCCTCACTGGCAGAACCAACCAGAAGCTGGAGGACAAGGGAGCTCCCATAT
GGGAGCCGGCCTGTGAGCCTTCAAGGGTATGCGGAGTCGTTTGTTCAAAATAGCCCCATTTCACAGATGAATCTCTACTGGCTCCATCTTCAGC
TTAGTGTTCTCTTCACTATGCTGTAATATCCCTTTTAACATAAATTCCTTTAAATTTCCTTTACTCACTCTGTTCCAATTCTGAATTCTCATGG
GCATGATACACTTGGTGTTCATAGAGCCAAGGATTTGTTACGCCTAATTACTAGTTTTTGAGTCAATTTGGTGGACTTTTACTGTCCTGCTGAT
AGATGACGTGACACGTCTTCCCAGTGTTGGGAAGCTCTTTATATTCATGCCCTCTCCGAGTATTTTTCCAAGCGCTGACTCTGCCACTTACTAG
CAGTTTGAACTTAGACAAGTCACTCACATCTCTGAGCCTTTTTTCCTTATCTGGGCAATGAGTTCTTTCCCACAGTGAAACTTTTATTAAGTAA
TGCACTATACAAATGTTTGTTATTATGTACACTTTCCACAGTCCAGTGATGCTTGCTGTGTAGTGTGTTGAATGCCCATCTTGCTGATAATGTG
CACTGCCAATTACACAGTTATATTGAGAGGTCACTTAATTATTAATGACAAGAGTGAACTCAACATCTTACAGAACTGAAATAGATATTTATAT
TTCCAACACGCTTGTAACCCAATAACTAGAGACTAAAAACCTAACTTCATGAAAAGGAGTGTAAATTTAGCTGTTATGGTCAAATACTCACACA
GCACACCCAGTTCACAAGCATCTCCAAAACGTTGATTGTTCTGATCAATTTTGAAAATCATCCTGCTCTCTACAGCAGGAATTCAGGTAGTACA
AGTGAAAACAGTATTATATACTACTTAAATGAAAGGCTGCCCTTGTGAGTTACTGTGTGTGAAGTTGCTTCTGCGTATGTCCATCCAGAGAACA
GGAGAGACACTGAGAAGACTGATGTGTCCCCGACCTCTAGATGGGACTGAAAATTCAAGATAAGAGAGATGCCCAGGGATAAACTTCCTTCTTT
CTTTAGAAATTATACAATATTAGAACTGAAAGGGACATTAGATATCATCAGGGCTTCCCATAAGGGGCTCACTGTCTTGCTTTATTGAAAGAAA
ATCAGTTTTGAAGCCAGACCAATCTTTTTTTTTCTTTTTAAATATAGGTGGTGTCTTGCCATCTTGCCCAGACTGGTCTCAAATTCCTGGGTTC
AACCAGTCCTCCCACCTCAGCCTCCCAAAGTGCTGGGATTACAGGTGTGAGCCACCGCACCTGGCCACCAATCTTGATTTGAATTCAGACCCTA
CCCTTTACCAGCTGTGTGACTTTGTATGTTTTACTTAATCTCTCAAAGCTTTATTTTACTTATATTTTAATTATACTGGAGATAAAAATGCATT
TCAAGTCAAGGTTGTTATTGTGAAGATTCTGCAAGTAAATACATTCTTGCATCTCTGTAAAATGTTGCCGTGCCTGTTGATTAGATAAGTCACC
ATGATGACTTATGATCAATTAGAGAAAAATGTCTGTTACCCAGCGATGGGAAAGTGAATACCTCCGAGACCCATGTCCCCAATTGTGTGTTAAA
ACTATTCTATGTTAAAAACTTACTTTAAAACTCAATTATATCTTCTCAAAATATTACTAATCATTCTAACATATTTTAGTTAATATGTTTCTCC
TTGTACAAATATATATGTGGGAAGTAGAGGGCATATTTTTCCTTAAGCTTGCTGCTTTCTAATCCAAAAAAAATGTGATGTGCAGGTCTATTAC
ACTCCACTTAAAATACTTACAACTTTGGGGCTTATTTTTCTCCTTTGAACCATTACTATTATAAAAATCAATTCTTCCTTAGTCACCAAGTCCT
CATCCTTGAGGAAAGGAAGTCAGCTAAAATACAAATCTCTTTGTAAAAGTAAAGTTTAGCGACTTGGGAGGCTGAGGTGGGAGGATCCCTTGAG
CCCAGGAGTTTGAGACCAGCCTGGACAACACAGAGAGACCCCATCTCTGAAAATAAATAAATTTCAAAAGTAAAGTTTACTTATATACAATTAT
ACCTAGTAACTTTGCTCCCTATAGCAAAAGGAAGCTAAACTATAAATCTTATGGAACTTGTTAGTGTTCTGGTAATAAACAGTTAATAATTTTT
TAAAATTAATATTCAGTGAAAATAATCTTGGCCAGGTGCGGTGGCTCATGCCTGTAATCTCAGCACTTTGGGAGGCTGAGGCAGGTGGATCACC
TGAGGTCGGGAGTTCGAGACCAGCCTGGCTGGCCAACATGGCAAAACCCCATCTCCATTAAAAGTACAAAAATTAGTGGGGTGTGATGGTGCGT
GCCTGTAATCCCAGCTACTTGGGAGGATGAGGAACAAGAATCACTTGAACCTGTGAGGCGGAGGTTGCAGTGAGCTAAGATTGCACCACTGCAC
TCCAGCATGGGCGACAGAGGAATGCTCCATCTCAAAAAAAAGAAAGAAAAGAAAGTAATCTTGATGCTAATATGGAAATGAAAGAGGTGGTTTG
ATATTTGTCTTTACACTATATTTCTTTAATTCAACTAGTACATGTCGAGGCTCTATTTTGTGCCAGGTGCTGTGTCAGAAGTTGGGGTTAGAGC
TCAGAACAACATAGATCAAAGTCCCTGCCCTCTGTGAGCTTATGTTCAGATGGGAGATACAGATGAGTAAAGGTTACTACAATATAATCAGAAT
AGGGATAACGGAGACCACGTAGAGCACCAGATCAAGACGTGGAGGAAGAAGACAGGTTTCCTACAGAACTGACACCTAGGCAGGACTGACAGAT
AAAGTCAGGCAAAAGTTGGAAAGGACAGTAGAGTAAGATGTGACATCTTAAACTTGCTGAATAAAAACACCCAAATATAACAAGTTATCTTTAA
ATTCATGATGATATATTGTATTTGGACATTCAGGCAGTTCTACTACCTGAGTATATCTAGTTAGTAAATTTTTTCCCCATGCTCCAAACAACAG
TTTCACACCCCTTTCTCTCAAACTCTCCATCCCGTTTCTACCCAGTCCCCGCAGATAACTTTGACTCATACACGAATGAGAAAAGAGAAGCCGT
CAAGAGCCACACCTGCATGCCTACTCTGTCTCTTGTTAGCATGGGTGAGCATGCCCAGCTGCAGTCATGGACTGGCCCATCCATCTGGCCTCTG
CAAGCCATTCCTCTGTATCCTCAGGGGTTTCATCTTGTGAATACTCTCTCTCTCCCCGACATCATCATTGTCTTCCTCTCTCCCAATTATTCCC
TCACATTAACATGGTCTACAACTTCCAGCAGGAAAGACAATGTTCCCTCATCAGTATAGTTTAATTTTTTTTCTTTTCTTTTTTCTTTTTTTTT
GGGGGAGTGGGGACAGAGTCATGCTCTGTCATACCAGGCTAATCAGTATTGGCACAATCAGGGTTCACTGCAGCCTCAGCCTCCTGGGCTCATG
TGATCCTCCTACCTCAGCTTCCCAAGTAGCTGGGACTACAGGCACGTGCCCCCAAGCCCCACTAATTTTCGTACTTTCTGTAGAGACAAGGTTT
CACCATGTTGCCCAGGCTGGTCTCAAACTCTTGGACTCAAGTGGACAATATCCCCTTATATACCCCATCATACCACCCCATTTATCTGCTCCTC
AGCAAAACTGCTTAAGGGAATTCTGTATGGCAGGTCCCCACTTCCCCAGGTTGCCTCCCCTCTGCCCACTCCACATGAACTTTCATTGCCCCCA
CTCCTCCATGCCTGCCTCTTCAATGTCACCAGTGACCTCCACTTCGCCAAATGCTGTAGTCACTTCTTGGGCCTCATTTTACTTTACTTATTGG
CAGCAGTCAACAGAGTTGACACCCCCTCTTTCTCTAACCATTTTTCTCTCTTGGCTTCAGGCATGACTCTCTTGGTTTTCCTGCTTCTCACTGG
TTCCCTCTGTTTGCTGCTCCTCCTCACTTTGGGCCTCAAGTGGCCCTTTCCTCCTTCTCTCTCCATCATTCTTCCTAAAGGATTTCATCAGTGC
TGTGGCTTTAAATACCATCTCTTCACCCCTGAGCCCACTCTGAAATTCTAGACTCCTGCAGCCTATTCCATGTTAACATCTTCCCCATATAAGC
ATCTTAGATTAAGTATGAACAAAACAGAACTCATAATCATTTAAATGCTTTTTTCTCATTAGCAGTAATTGGCCCTTGTATCTTAATTTCTTCA
TTTCTCAAGCCAAAAATTTGTCATCCTTGGTTTCTCTCTTTACCTTATTACCCTCCTTACACTCAAATGCTCAGAAAGACCTGAATATTCTATC
TGCAAAATGTATCTTAAATGTGTCTGCTTTTTCTATTACCTGTAATAATCTTGTTAGTAATGACACAATATTTATGAAGTGCTCTCAATATGCT
AGGGAAGAAGAAAGAGGGAGAGAGAAAAGAAGAAGAAAGGTGATGTCTGAGAGGTGAGTTTTGAGAAACTGAAAGAGAAACCTGGAGGAAAAAA
AAAGATACGAGGCCGGAGAGAAAAGCGGTGTAAATAGAGCTTGAACTTTGTCCGTAGAGATATGGGGAGTTATGGAATGGTTTCTGCAGTGATA
TAAAATTACTTATTTATAGCTGATTCATATAAATAATTTTGTTCAAATCCCTTTTTCATAGACAGTATAAAATTAAACCTTAGGTTAGCAGGGC
CAAATGCTGGCAGGGCCTGAATCTTATACTCAGTTAATTGGATTGTACATAGTGCACTTCCTACTGCTTCTTAATTTCAAAAGAATGTGCTTTA
ATCCCAGAAAAATCTTATTGAAAACATATCTTTTGATCCGAAGTTCATAAGGGCTGTTTTCACGTTTTATAGTCCATAAAGTAATTAGTTCAGG
AAGAACTGATTACTTAAAACTCTTTTCTTTTTTAAGAGCAGAATATTATCAACAAATAATTTCTCTAAAAGTTTCCTCATCATTTTCAAAAGCC
AAATCTTCTACTGAATTTTTCGTATGGAATAATCTGATTATATTTTGATATTTTGGTCATTAAGAAATTCTGCCAATATTTGTTCTTGAAGGTG
TTCTTAGCTTAATTTTATATTTTCTTTTACTCTTTAATCTAGAATGCATTTGTAGAGTCAATGATCCTCAGAAACACATTGGAAATTTGTTTAA
AATGGAGATTCTTGACGTCACCCTCGGATTCTGATTTATGAGTCAGGTGTAGACCTGGGAAGGTTTAACAAGCTTGCAGCTGATTCTAATGCAG
GTGGTCTGAGAGGACCACACCAAAACACCCTGCCTTACTCCCCCAACCCTGTGTTTTCTGTCTAAACACTCCCCAATAGCTTTCAATTCCTTTG
GGGCCCATAATTTTTAATTAAAATGCCCATTTGTCTATAGAGTATTAGAAAAATATTAAGACCCTAACTATAAGTAGATTTTCTTAAATCCAGG
GAATTATTATGTTCCAGATGTTGGTACTAATAATGGCTAACCTCGGCCGCTGATCCATCTTTTGTTTGATTTTTATAATATATGGGGAAGCATA
ATTTTGTATATATCTAAACCTCTTTAGAAATATGGTTCAATTGCATATGATCTGTCATAAGGGAATTTATATCTCACGAAGTTAAGCGCATAAA
CAAAACAAGACCAAACACACACACACACACACACACACACACACACACACACATTTTCATTCAGAAATCCTTGGTTCTAAACCAGTCTCTAATC
AACATGCCAAAAACCCTTGTTTCAAATCACTTTACTCCTCTGTGCTTTGGGATTTCTATTTAAATGGGGTAAGTATTTAATCCTGTATATAGTT
ACAAAGCCCAGGGAGAACCTCAGATCAAAGACAAAATTGAAAGAAAGCTTTAGTAGGCCAGAGACATTGGAGTACCAAGGATATAATTGTGAGG
TTTTCCTGCTAGAAAACATCCTCTGAAATGAACTTATAAGTTAACAAATCACCCTTCGCCTGTGTTTGGGGGCAGATGTTAGGCAGAGTCAGGA
TTTGTTTCCATTCGGTCACTTTTCTAAAGAATACAAATCCATCCACCTATTCTTTAATGGTCCTAAATAGCTAACAATGGACAGACAACTCCCT
TCCCTTCCTTGCCCAGTTTCCGAGTCAAGGCGCGCAGTGTTCAGTGTATGACCTGAAGCCTTCTGGAAAGTGGTGATTGGTTAGGGTACTCGCC
AGTTTGGCCTTACTTTGAACATCCCAAATTTATTTCCTTCCCCATTTCAAATATTCCGAAATGAAAGACATTAGGAGCACGTATTCTGGTTAAA
TACGGTGATTGAAAATACGAATGAATGCCACTCCATCATGAAATTTCACTAAATGACAATGAGTCAGCAAATAAGGGGCATAAATATACAAGTT
AAAGAGAACAACAGGTAAGAGATTATTTGTCAAGAGCCTGTAACTCATTTATTTATTCAACAAATATTTACTGAGCACCTCCTCTGCAAGCAAC
ACAGAATTCAAAACAATTAAATATGTATTACATATACAGCATATATGTTTACATATAAACAATATATAACATATATAAAGTTACTGGCAGAGTA
ATATTTTCTTGGATAGTCACAGATGTATTAAGAAACAGGCTTGTTTCAAATTTGCTTTTTTAATAACCATATTTCTTTAACATATTCTTTTTCA
TCTTTTACCATTTCTGACATCAGATTGTGTCTTTAACATGGTCTGATTTTTCTTTTGTTGTAAACTTATTAAACCCATAGAGTCTTATAATTGA
TGATACCATAAAATAAAGGAAAAGCTTGGAAATTAACCAGCAGAAATTTCAGAAAGGACCCCTTAGGTTTCTTTTTTTTTTTTTTTTTCTTCTG
AGACAAAGTCTCACTCTGTCACCCAGGCTGGAGTGTGCAGTGGTGAGATCTCAGCTAACTTTCATCAGTAATGAAACCAGCAGGTGTTCTGCTC
CCCAAAAACTTTGTCCACTGTCTCCAGACACTCAGAGCCACATGCTTAACTGTGGCTCATGGGTTAATGCATGTCTGAACTTTTTCAGGGTGGA
AACTCCCTTTTTTCTTTAGTGTCTAACCCAATGCCCAACATGACATAAGTGCTCAGTAAATATTCAGACAATCCACGTATGAATGTGGTTGAGC
ATCTGTGAGTGGTCTCACCTCCACCCTACATGTGTTACTATGGTCAACCACTTCATCTCTGTGGGTCTCTGCTTGCTCAGCTATAAATTATTTG
GGATCAGCTCAGCCATAGATTCCCTCCAACTCTAATTATCTCTGAGTAAATGAAGTCATGAAAATATGGGAAACTGAGGCAGGAGAATAGGGTC
TAGAGGCAGGGAACCTAGGGCTGATTCACAATTAATTCCTAGAACTAAATCAAAAGGAAAACCCCCAACTTGCCATGCCCAACTATCAAAAGGA
CCAGAGGCTACTCCCTTTACAACCCCCCAACTTTTTCCGCATGGCAGATGAAATATTGAAAGTACCTCTGATTGGTCCCCTTCTGCAACCAATC
AGGTTGGTCATGGGTCAAGTCTTCATTTGCATAGGAGTATAACTTTGTATATACTCTATTTTATCCCCTCCCACAAGCAATCAGACTGATCATG
GGCCACCACTTCATTTACATAGGGTGTACACCAAGTAAGCAATGGGAAACCTCTAGAGGGTATTTAAGTCCCAGAAAATTGTGTAACTGGGCTC
CTGAGCCACTTGCTCAGGTCTGCTCCCACCCTGTGGAGTATGCTTTCATTTTCAATAAATCTCTGCCTGTGTTGCTTCATTCTTTCCTTGCTTT
ATTTGCACATTTTGTCCAATTCTTTGTCCAAGACACCAAGAACCTGGACACCCTCTACCGGTAACAAAACTACAGTGATTTTAGAGGGTTTGTT
TGAAGTGTGTATTGCTCATGAAGGTAATGCCTTTGATGATAACATTTGCTAAACACCACGTAAAGGAAGGGAGGCATTTCCACACTTTGGGTCT
CCTCCCATCTCTATCCCCAAGCCTTAAAATTTCTGCTTAAATGGGAGTCGAAGGAACCCAACAATACTCAGTTGTGTCACTTAAAGGCTGTGCA
ACATCGGGAAGCTTTCTGTATGTCTTACAGCTAGTTCATATCACAAGCCAGCCGTTTCATCATGCAAAAGTCTAAAACAACTTACACCTACTTT
CTAACATTCTTACGGAAAAACCAATACTGTTGAACAGAGGTGGACAAATTATAGCTCCTGGCCAAATCTGGCCCACTTCCTGTTTTAATAAATG
CAGTTTTATTGGAACACAGCCACACCCTTTTATTTCCCATATTATCTATGGTTAGTTTTCTGCTGCAATGGTGGAGTGAAATAGTTGCAACAGA
GACCATGTGACCTCCAAAACCTGAAATATTTACTATCTGGCCCTTTACAGAAAAAAAGTTTGTGACCCCAGCCCTAGAAGAAAAGTAATCAAAA
ATCACAATGGCAGTATTAGTGGTGAAGATAATGGATTCTGCTTCAGTTAAACAGTGTAACTGAGCTTATGATTTTTCTTTTCCTTCTTTTTTTT
TTTTTTTTTTGAGATGGAGTTTTGCTCTTGTTGCCCAGGCTGGAGTGCAATGGCGCGATCTTGGCTTACCGCAACCTCCACCTCCTGGGTTCAA
GCGATTCCTGCCTCAGCCCCCTGAGTAGCTGGGATTACAGGCATGCTCCACCATGCCCAGCTAATTTTGGATGTTTGTAGAGATGGGGTTTCTC
CATGTTGGTCAGGCTGGTCTTGAACTCCTGATCTCAGGTAATCCACCTGCCTCGGCCTCCCAAAGTGCTGGTATTACAGGCGTGAGCCACCGCA
CCCAGCCGTGATTTTTCTATTACCTAGGTCCTTTTGTTGGCTACACACCATGACTGCTTCTGTTTGGGAGTTGAGAACATCTGGTAGGTTAAAT
TGGCAATATTTGACTGATTTGACCTACAATATGTGGTCAACTTCATGTGACTCAACCTAATACTTTGGCTTCCTTCAGTGTGTTCAGTTTCAAT
TCCCCGCTCCCTTTGTAGTCTTCCAGCTACCTCAGTGATTCTTAATCTTAGCTGTGTATTAGAATCATCTGAAGAACTTAAAAAAAAAAAAATC
AATGCCCAGGTCACACCCAGGACCAAGTGAAGCCGAATCTCAAGGGGGAGAAACCAAGCCCTTGGTAATTTTGGAAAGCACCCCAGGTGATTCT
GAAGTGCAGTAAGGCTTGAGAACTATGGAACTACCAAGAGCTTTTGGCAATACGCATGTGCTAGGTGCTATAGACTCCTTCCAATTGGAGAAAA
GAATGTGGACAATTGTAATAAATAATTGGGGCAAGCCGGCATAGTGGTTCTCAAACATTAGCATGCAACCAATCACCTGGTGGGCTTGTGAAAA
CACAGATTTTTGGTGCCAACCCCTCAGTTTCTGATTCTGTAGGTCTG
HUMAN SEQUENCE - mRNA
GGCGGCAGCGCCCTGCCGACGCCGGGGAGGGACGCAGGCAGGCGGCGGGCAGCGGGAGGCGGCACCCCGGTGCTCCCCGCGGCTCTCGGCGGAG
CCCCGCCGCCCGCCGCGCCATGGCCCGAAGACCCCGGCACAGCATATATAGCAGTGACGAGGATGATGAGGACTTTGAGATGTGTGACCATGAC
TATGATGGGCTGCTTCCCAAGTCTGGAAAGCGTCACTTGGGGAAAACAAGGTGGACCCGGGAAGAGGATGAAAAACTGAAGAAGCTGGTGGAAC
AGAATGGAACAGATGACTGGAAAGTTATTGCCAATTATCTCCCGAATCGAACAGATGTGCAGTGCCAGCACCGATGGCAGAAAGTACTAAACCC
TGAGCTCATCAAGGGTCCTTGGACCAAAGAAGAAGATCAGAGAGTGATAGAGCTTGTACAGAAATACGGTCCGAAACGTTGGTCTGTTATTGCC
AAGCACTTAAAGGGGAGAATTGGAAAACAATGTAGGGAGAGGTGGCATAACCACTTGAATCCAGAAGTTAAGAAAACCTCCTGGACAGAAGAGG
AAGACAGAATTATTTACCAGGCACACAAGAGACTGGGGAACAGATGGGCAGAAATCGCAAAGCTACTGCCTGGACGAACTGATAATGCTATCAA
GAACCACTGGAATTCTACAATGCGTCGGAAGGTCGAACAGGAAGGTTATCTGCAGGAGTCTTCAAAAGCCAGCCAGCCAGCAGTGGCCACAAGC
TTCCAGAAGAACAGTCATTTGATGGGTTTTGCTCAGGCTCCGCCTACAGCTCAACTCCCTGCCACTGGCCAGCCCACTGTTAACAACGACTATT
CCTATTACCACATTTCTGAAGCACAAAATGTCTCCAGTCATGTTCCATACCCTGTAGCGTTACATGTAAATATAGTCAATGTCCCTCAGCCAGC
TGCCGCAGCCATTCAGAGACACTATAATGATGAAGACCCTGAGAAGGAAAAGCGAATAAAGGAATTAGAATTGCTCCTAATGTCAACCGAGAAT
GAGCTAAAAGGACAGCAGGTGCTACCAACACAGAACCACACATGCAGCTACCCCGGGTGGCACAGCACCACCATTGCCGACCACACCAGACCTC
ATGGAGACAGTGCACCTGTTTCCTGTTTGGGAGAACACCACTCCACTCCATCTCTGCCAGCGGATCCTGGCTCCCTACCTGAAGAAAGCGCCTC
GCCAGCAAGGTGCATGATCGTCCACCAGGGCACCATTCTGGATAATGTTAAGAACCTCTTAGAATTTGCAGAAACACTCCAATTTATAGATTCT
TTCTTAAACACTTCCAGTAACCATGAAAACTCAGACTTGGAAATGCCTTCTTTAACTTCCACCCCCCTCATTGGTCACAAATTGACTGTTACAA
CACCATTTCATAGAGACCAGACTGTGAAAACTCAAAAGGAAAATACTGTTTTTAGAACCCCAGCTATCAAAAGGTCAATCTTAGAAAGCTCTCC
AAGAACTCCTACACCATTCAAACATGCACTTGCAGCTCAAGAAATTAAATACGGTCCCCTGAAGATGCTACCTCAGACACCCTCTCATCTAGTA
GAAGATCTGCAGGATGTGATCAAACAGGAATCTGATGAATCTGGATTTGTTGCTGAGTTTCAAGAAAATGGACCACCCTTACTGAAGAAAATCA
AACAAGAGGTGGAATCTCCAACTGATAAATCAGGAAACTTCTTCTGCTCACACCACTGGGAAGGGGACAGTCTGAATACCCAACTGTTCACGCA
GACCTCGCCTGTGCGAGATGCACCGAATATTCTTACAAGCTCCGTTTTAATGGCACCAGCATCAGAAGATGAAGACAATGTTCTCAAAGCATTT
ACAGTACCTAAAAACAGGTCCCTGGCGAGCCCCTTGCAGCCTTGTAGCAGTACCTGGGAACCTGCATCCTGTGGAAAGATGGAGGAGCAGATGA
CATCTTCCAGTCAAGCTCGTAAATACGTGAATGCATTCTCAGCCCGGACGCTGGTCATGTGAGACATTTCCAGAAAAGCATTATGGTTTTCAGA
ACAGTTCAAGTTGACTTGGGATATATCATTCCTCAACATGAAACTTTTCATGAATGGGAGAAGAACCTATTTTTGTTGTGGTACAACAGTTGAG
AGCACGACCAAGTGCATTTAGTTGAATGAAGTCTTCTTGGATTTCACCCAACTAAAAGGATTTTTAAAAATAAATAACAGTCTTACCTAAATTA
TTAGGTAATGAATTGTAGCCAGTTGTTAATATCTTAATGCAGATTTTTTTAAAAAAAAACATAAAATGATTTATCTGGTATTTTAAAGGATCCA
ACAGATCAGTATTTTTTCCTGTGATGGGTTTTTTGAAATTTGACACATTAAAAGGTACTCCAGTATTTCACTTTTCTCGATCACTAAACATATG
CATATATTTTTAAAAATCAGTAAAAGCATTACTCTAAGTGTAGACTTAATACCATGTGACATTTAATCCAGATTGTAAATGCTCATTTATGGTT
AATGACATTGAAGGTACATTTATTGTACCAAACCATTTTATGAGTTTTCTGTTAGCTTGCTTTAAAAATTATTACTGTAAGAAATAGTTTTATA
AAAAATTATATTTTTATTCAGTAATTTAATTTTGTAAATGCCAAATGAAAAACGTTTTTTGCTGCTATGGTCTTAGCCTGTAGACATGCTGCTA
GTATCAGAGGGGCAGTAGAGCTTGGACAGAAAGAAAAGAAACTTGGTGTTAGGTAATTGACTATGCACTAGTATTTCAGACTTTTTAATTTTAT
ATATATATACATTTTTTTTCCTTCTGCAATACATTTGAAAACTTGTTTGGGAGACTCTGCATTTTTTATTGTGGTTTTTTTGTTATTGTTGGTT
TATACAAGCATGCGTTGCACTTCTTTTTTGGGAGATGTGTGTTGTTCATGTTCTATGTTTTGTTTTGTGTGTAGCCTGACTGTTTTATAATTTG
GGAGTTCTCGATTTGATCCGCATCCCCTGTGGTTTCTAAGTGTATGGTCTCAGAACTGTTGCATGGATCCTGTGTTTGCAACTGGGGAGACAGA
AACTGTGGTTGATAGCCAGTCACTGCCTTAAGAACATTTGATGCAAGATGGCCAGCACTGAACTTTTGAGATATGACGGTGTACTTACTGCCTT
GTAGCAAAATAAAGATGTGCCCTTATTTT
HUMAN SEQUENCE - CODING
ATGGCCCGAAGACCCCGGCACAGCATATATAGCAGTGACGAGGATGATGAGGACTTTGAGATGTGTGACCATGACTATGATGGGCTGCTTCCCA
AGTCTGGAAAGCGTCACTTGGGGAAAACAAGGTGGACCCGGGAAGAGGATGAAAAACTGAAGAAGCTGGTGGAACAGAATGGAACAGATGACTG
GAAAGTTATTGCCAATTATCTCCCGAATCGAACAGATGTGCAGTGCCAGCACCGATGGCAGAAAGTACTAAACCCTGAGCTCATCAAGGGTCCT
TGGACCAAAGAAGAAGATCAGAGAGTGATAGAGCTTGTACAGAAATACGGTCCGAAACGTTGGTCTGTTATTGCCAAGCACTTAAAGGGGAGAA
TTGGAAAACAATGTAGGGAGAGGTGGCATAACCACTTGAATCCAGAAGTTAAGAAAACCTCCTGGACAGAAGAGGAAGACAGAATTATTTACCA
GGCACACAAGAGACTGGGGAACAGATGGGCAGAAATCGCAAAGCTACTGCCTGGACGAACTGATAATGCTATCAAGAACCACTGGAATTCTACA
ATGCGTCGGAAGGTCGAACAGGAAGGTTATCTGCAGGAGTCTTCAAAAGCCAGCCAGCCAGCAGTGGCCACAAGCTTCCAGAAGAACAGTCATT
TGATGGGTTTTGCTCAGGCTCCGCCTACAGCTCAACTCCCTGCCACTGGCCAGCCCACTGTTAACAACGACTATTCCTATTACCACATTTCTGA
AGCACAAAATGTCTCCAGTCATGTTCCATACCCTGTAGCGTTACATGTAAATATAGTCAATGTCCCTCAGCCAGCTGCCGCAGCCATTCAGAGA
CACTATAATGATGAAGACCCTGAGAAGGAAAAGCGAATAAAGGAATTAGAATTGCTCCTAATGTCAACCGAGAATGAGCTAAAAGGACAGCAGG
TGCTACCAACACAGAACCACACATGCAGCTACCCCGGGTGGCACAGCACCACCATTGCCGACCACACCAGACCTCATGGAGACAGTGCACCTGT
TTCCTGTTTGGGAGAACACCACTCCACTCCATCTCTGCCAGCGGATCCTGGCTCCCTACCTGAAGAAAGCGCCTCGCCAGCAAGGTGCATGATC
GTCCACCAGGGCACCATTCTGGATAATGTTAAGAACCTCTTAGAATTTGCAGAAACACTCCAATTTATAGATTCTTTCTTAAACACTTCCAGTA
ACCATGAAAACTCAGACTTGGAAATGCCTTCTTTAACTTCCACCCCCCTCATTGGTCACAAATTGACTGTTACAACACCATTTCATAGAGACCA
GACTGTGAAAACTCAAAAGGAAAATACTGTTTTTAGAACCCCAGCTATCAAAAGGTCAATCTTAGAAAGCTCTCCAAGAACTCCTACACCATTC
AAACATGCACTTGCAGCTCAAGAAATTAAATACGGTCCCCTGAAGATGCTACCTCAGACACCCTCTCATCTAGTAGAAGATCTGCAGGATGTGA
TCAAACAGGAATCTGATGAATCTGGATTTGTTGCTGAGTTTCAAGAAAATGGACCACCCTTACTGAAGAAAATCAAACAAGAGGTGGAATCTCC
AACTGATAAATCAGGAAACTTCTTCTGCTCACACCACTGGGAAGGGGACAGTCTGAATACCCAACTGTTCACGCAGACCTCGCCTGTGCGAGAT
GCACCGAATATTCTTACAAGCTCCGTTTTAATGGCACCAGCATCAGAAGATGAAGACAATGTTCTCAAAGCATTTACAGTACCTAAAAACAGGT
CCCTGGCGAGCCCCTTGCAGCCTTGTAGCAGTACCTGGGAACCTGCATCCTGTGGAAAGATGGAGGAGCAGATGACATCTTCCAGTCAAGCTCG
TAAATACGTGAATGCATTCTCAGCCCGGACGCTGGTCATGTGA

TABLE 7

MOUSE NOMENCLATURE
ICSGNM Sox4
Celera mCG11673
HUMAN NOMENCLATURE
HGNC SOX4
Celera hCG36747
MOUSE SEQUENCE-GENOMIC
ACTATTGGTAACTGTGATGGTTAACTTTGCCAGTTTCACTGGGCCATTAGGTACCCAAATATTTGTTGGAACATTCTAGAAGTATCTATGAGCA
TATTTTGGGTTGAGTTTAATATTTCAGTCTATAGGTTGAGTAAAACAGACTGCCCTCTGTTGGGAACAAAAAAAGGGGTCCAACATCCAGCCAG
AGTTCCTGTGCTCTGAGCAGGCAGACGCAGGCAGAACTGCCAGACACTTTCCACTCAGCCCCAGGTGGACATCTAAGCCTCTGACCCCACTCGG
CGGGGGGTGGACAAGGGGCAGCCCCCAACTGGGGACCCTGAGGTGACACCCTGTCGTCCCGGGGTTATAGGAGAGAGGGAATAGGGAAGAGGTT
CCCAACACTGACCAGAGTCTTAATGGAGCAGAGACTCTCTATGGTTTAAGAGCTTTATTATAGAAATGCAGGGAGAAAGAGAGAAGGTAGAAAA
GGAGAGAGAGAGAGAGAGAGAGAGAGAGAGAGAGAGAGAGAGAGGGAGGAAGAGGAAGAGGAAGAGGAAGAAGAAGAAGAAGAAGAAGAAGAAG
AAGAAGAAGAAGAAGAAGAAGAAGAGGAGGAGGAGGAGGAGGGAGAGGAGGAGGGAGAGGAGGAGGGAGAGGAGGAGAGAAGAAGACAAGGAAA
AGAAAGAGAAGAGAGAAGACAAAGAGAGGGGAGAGGGGGGAGAGAGTGAAGGGTAAGAAGTAAGAGCATAAGAGTAAGAGAGAGAGGTGGGGGC
TGAACAGCCCTTTTTATGGTCTTTACTGCTGCTAGGTAACTGGGGAGGAGTTTAACCTGAAGGTCAGAATCTTGGGACATTGCTTATGTGACTA
CTGACCATGCTTCTCTTGTGGAGGCTGTGGGAGGTGGTAACTTAGGCAGGGGCCAGAGCTCCAGGAGCATGAGGGAACGCCTACTGTGTCATGA
AGGTAAATTATGACCATCAGGGTTCAGACCTCAGCTCGACCGGAGACCAGCCTGCAATTCCCCACAGCCCTCCCTAAAGTGGGTTAAAAGACAG
AAAAGAATTAAATATCTGATCCTCTCCCATATCTACAAGACGTAACTTCCCTTGTCCAACCATCTTGTAGATTGGATGTTGTTTTTCCCTTGCT
TTTTAATTAGAACTATGTCATTGGTCCTCCTGTGTCATAAGTCTACAAAGTGCATGTGTGCTGGCTAATCTTGTGTCAACCTGACAGACAACTA
GAATTATCTGAAAGGAGGGAACCTTAATTGAGAAAATGCCTCCATAAGATCCGGCTGCAAAGCATTTTCTTAAATTGTGATTGATGTGGCCGGG
CCCAGCCCATTGTGGGTGGTGCCATCCCTGGATTCTGGGCTCTATAAAGAAAGCAGGCTGAGCAATCATTGAGGAGCAAGCCAGTAAGCAGCAC
CCCTCCACGGCCTCTGCATCAGCTCCTGCCTCCAGGTTCCTGCCCTGCTTTGAGTTCCTTCCTGTCCTGACTTTCTTTGATGATGAACAGCAAT
GTGGAAGTATAAGCTGAATAAACCCTGTCCTCCTCCCCAACTTGTTTTTTGGTCATGGTGTTTCATCACAGCAAGAGACCCTAACTATGACAGT
ATGTCTTAGGAATTATCAGCCTCCATAATCAGGAGCAAAATTCCTTATAGCAACTCACTAATATGCCAATGGTCAAAGGAAGTTAGTGACCAAA
TCCAGAGAGTAAGGGGTAGAAAAGTCTTACACGGGAGGTACCATTGCCAGGAAGGTTGGATTATCAAGACTCCCAACACACTGGCTGCCAACAA
GGAAACCCCAAATATCGGCAGAATTCTCACTGATTGGCACTTGCCTGGTAACTATAGTNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNTGTGTGTGTGTGTGTGTGTGTGTGCGCGCGCGCGCGCGCAACATGTGTTGGCATGTGTGCCACTGG
TGTAGGCATGACAGTCAGAAGAAAACATTAGGGAGACAGCTCTCACAAAGGGTCTCTGTTAGTTTTCCTGCTGTGCTTACTATCACAGGTTAGA
TAGCTCACAACCACTCAGGAGTCTCCTGTCTCTACCACCAGCTCACAGCCACTAGGAGTCTCTGCCACCAGCTCACAGCCTTTCAGGAGTTGCC
TGTCCCTGCCTCCTGTCTAACTTTCGAGTGCTGAGATGACAGGTACCTACATCTAGCTCTCTTATGTAGGTCCTGGGGAGTAAACTCAAGTCAT
TAACCTTGTTGTAGCAAATGATTTTACCCACTCAACTGATGGCCCAGTTGCCTTGATGGTCTGTGTGGTTGGTTGGCTGGACTTTTTGTTTCGA
GATAGGATCTAAGCTAGATAACTTAGGCTGGCCTTGAACTCATTATATAGCCCAGGCTGGCCTCCAACTCAGTGCTTGCCATACTTCAACCTCC
CAGATGCTAGGACTATATGCATGAGCCATCAAGCCTAGCACCGTGCATATTTAAATTGTATGGTTTGTATTATACACTATATAGATTTTAACAC
ATGGTACATTGGGAATGATGTCGATTTTTAAAATACATATATAGTAGGTAATGTCACACACAAATTTTCTTTCATTTAAGGTTACATTACACAA
TGCATGGTGGCACAAGCAAGTCTTTAATGCCAGCTCTTAGAAGCAGGAGGTAAAAGAATCCAGAGTTCTAAGTCATTCTAACTGCTCAGATAGT
TCAAAGCCAGCCTGGGTTGCATTCAAACTTGTCTTTAAAAAAACAAGTGACTCGACATTTGTACAACACAGTGATATTTCAGATGATGTTTGTG
AAATTGAGTTAAATTTGAGTGTTGTAAGGATGAGCCTTCATTTATTTGTCCCGACAAGCCAAATCAACTTACTTATGGAACTAACTTAAAGCAG
TCTGCTTCTGTAAGTCCAGGCTGTCCTTCAACTTGTGTTTCCACAAGCTCTTGCCTCAACCAAGTGCTGAGATTTAGGTGTGTAAACCATTTGA
AATTATTTTTCACTTGGGCTGTGTGGGACAAACAGCCCACAGAGTCTTGTGCTTCAAGGACTTCTTTTAAAAAAAAAAATCATAAGCTACTAGT
CTCCAGTTACTCGGCCAGACACCTCCCTCCTCTTGCCTTCAAATTTGCCTCTTTACAAAAGCTGCTTTGCTGGCAGTTCAGTATGAATTTATTT
TACAGCATATACAAGGTTTTCTGGTAGCTTGCAGTTCCTGAGAACTGCAACCGAAGGCTCTTAAGCTGAGCGGGGGACACACAGTAGGGCACTC
ACTACGTTGATTGGCAGTTGAAGTGGCAGTTGGTGTGGAGTCGTCGCTGCCACGGGTACTCTTATCTTTCCTGGCCACACCCCTGGGCCCAGGA
GGCAGCTCTGAACAGATGGTGAGTCTCCGGTGCCAGGTGTGCAGCGCGCCCTGCGCTGTGTGAGGCACAGGGCTCTTTGGTCCTTGCCGCACCC
CACCCATCCTATGCCCGCGAGCGCCCCCAGCGCAATGTGTGTCTCGGTGCTGTCCGCACGCGCGGTTAGCCGAAGGTGAAAGGCTGGGAAAAAA
GGAACCCTAGTGCCAGGCAGAACCCGCCACCGGGCCAGCTGCTCGTGGCGAGCACGTTCCTCCCCTCCTCTGACTCCCATCACTTGGCAGCCAC
CCCAATCGCCTGCTCGCCCCTCTGCAGCAGCACACACGCCTTCGCACTTGCTCCCCTCCCTATTCTCTTTTCTGACACTTCGTGGCTTACATGG
ACCACTCTCCAGCGGGATGCCCTTGGTCCCCGCCCGTCTCTTAGCCTTCAAGGTGGCTCTCCTCGTTCCCTAATATTTGGGGAACTCTAGGCGG
CTTTTTTTTTTTCCATAATCGTAATCATTTGCTTGCCTCCTGGGACTCCAAACTGTAATTGCTTTTACGTGTCTGTGGTCTCGAGGACAAGGAA
TCCACTGAGAGATGGTTTGGGGGTGCTGGGCTGGGAAGAGGATGCTCTATTCCGATTCCGAGTACCGTTTGCGGGGTGGGGGCTGCAAGGAGGA
AAGTGCGCTCAGCCTTTCCTTTATCCACCCTCCCCCCGTCGCCCTCGGCAAGGGCTTTGGGGCGCCTTCGAGGTCCTCTGTGGACACACAAACA
CCTCAATCTTCCTGATCAGGCTAGAGCCTGGAGGTGGGGAACTATCTGGTTCCTTCGTTCTGCCCCTGGGAAGAAGCCCTGGCCGCTGTGCGTG
CACTGCCCCTCCCGCAGTCACAGTGAGAGCTGAAGCGTGAGTCCCAGACCGATGCAGATGGCACGCTATTTAGTCCAGGAGAGGCTCACAGAGG
GTAAGGGGCTGCTGGGCACCCGAGTGCACCCAGGGAACCAAATGGCTGTCGGTTCCGGTCAGTGGCGGAACTGGGCCCCAGCGGCTAGGTCCAG
GCTGGAATCGGGGGGAGGGGTGCGTAGGGGGAGGGACTGGGCGAGATCCCCCCTCCTGGAGTGGCTGCTCCAGCTGCCACTTGCAAGGANNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNGGCGGGGGGCGTCACCAGCCCATTCCTGACCGTAAACCTCCTCAGA
AGGCAGTGGTGAAACGCCGCTGCGAGCGTCGCCCGAGCCACGTTTACAGTAGGAAAGACTTTCCTCCGAAAAGCACCACCGGAGTCGATTGCAA
TTTAGCTCCTCCCAGTAAGAGTCACAGAGAGGCAAGGCGAGGCCGGTGCACCGGGAGTCCTAGGCGAGCTTGGGCAGGACCCCTGTTGCTGAGC
TCCACGGGGCGGGCGTGCCGGGCTACGGAAACCATTTCTTTTCAGGAGCAGGGTTGGCCTTTGGGACTCCTGTGGTCATGCACCGTGTGTTTCT
GCCGCCGCCGAAGCAGAGCGCGCAGCTGGCGGAGAAACCGCATTCTCGCTCCTACCCGCCCGAGGGAGATGACCCGTGACCTCCCGAGTGAGGT
TACCAGATCCGAAAGGGGCGTCTCGGCTTCCCCAGCCATGGGGGTTGAGGGGGTGGGGGGTGGAAGGTGGCCCTGAAAAAAGTGGACGAGCAAA
CAGGGAGCAGCAATCGGTTACCCTAAACTTTGTCTTACGGGGAATGGGGGGATGCCCAGAATTCACCAGCACACCCACCGGTCCTTGAAACCCG
GAGAAGTGTTGGGGGTTCACTGGTTCAGGAGACCAGGAAGGGACGGTTGGATCTTCCCCCACGTTTTCCCACGTCGCTGCATGCTCAGGCAGGC
TCAGCGCTGCCACCTGGCGGCTGCCTCTCCCGGACTGGGAGGAGCTGTCTGCACGCACCAGTTCCACAGATCTTGAAGGGTGTGCCAGGCCTCT
ATCTGGGGAAAGGGCAGAAGCTCCAGTTAATGCTTCCTATCTATGTGGCCCGTGCAGACACACTTGTTTTCTGCTTGTTCTCAAGAGTAAGAGG
GAAATGCGTTGGCGTTGAACGGCATGGGTATAAAGAGCTGACTTGAGCCTGCTGGTTATGAGGGGTGAGAAAGTGGTGAAATTATTAGAAATAT
ATGTTTCTTATCTCTGTGTAGTCTAACAGTGAATATGAACGACAGCTTTCATTTATCCTGCTTAATATTTTTCGAATAAGAATTCAACACACTG
GTCTGGAGAGATAACTCAGCCCTTGATAAAGGCTAAGGCTCACAACCAAAACCTAAAAAATACTGTTTCAATACAGCCCCCCTTTCCTAGTCCA
GTTTCAATACAACTTCCTCAGCACCAAGAATGGAGTAGTGCAAGGGGTTGGGGGGTGGGGAGCAAAATCAGCCCCAGTGAAGGGTTGACCTGAA
AACAGGGCTGATGGCATTCAGTTTCCCAGAGCACTTCCAGTCAACGAAATTAACAAGAAGGCGTCCTCCACCTCTGTTTATTTGAACTGAATGT
TCAAAGCCAGAGGGGCTCACACACTCAGTTTCTACAGCCAAAGCAAAGCACGATTCTTGGAAAATAGTGCCAAGCCAGACTCGTGAAACTAATT
TCCATGCAGGTTTTCCTCCCTCTATGCTATCGGGACACATTAAGAAGTCGAGGTAAACCTTCATCCATACCCCATCCATACCATTCTGTAGATG
TGAGGTTTTATGTTGTGCAGGTGTGTAGGGGCAGGAGAAGCACATTAGCAGTTATTACCCTCTCATGTCGAATGGATTGCATTTAATGGAATAA
AATAAATATCTGCCTTAATTCAAGACGTGAAAGATCTAATATCATTTCCAATAAAGACCATTAGTTGATGATTCTAAGGCAGGACTTGTTTGCA
AAACAATCCTGGCTCTCTGTTCATAGAACTACAAGGGGAGCGGAGGGAAAGAGCACTGAGCACTGACTTACTAGGGGCCACCAAATAGCTAGTA
TCTGGGGGGAACGGGGAGGTCTCTGATGGAGAACTGATTACAAAGCTTAGGGCATTAGGTAATGAGACCCAGAAATCTTAATATCATGATTTGG
TATTAAGTCCAGTGCATTCTGGTGTATATGTACTTTAATCAAAAGCTATTTGTCACATCTTTTGTCACATGTTTCCATGCTCTAAGAACAAAAA
CAGTCACACTTCAATAACTCCAAACTGTCAGGTATATACTCTACAGGATTACATTGTACAATGGTGTAACTAGGTCAACGGATGCTACCAACAC
ATATTTTACCAGGTCTAAGAGAAACATAGACACAGGGGGTGCATCTCATAGTCAGGGGGACAAGGAGGACCTTTAGCAGCTCGTGAGTTGGCTC
AGCCGGTACAGGCCTCTCTAGGCCAAACCTGGCAATGTGAGTTCAATCCCCAGTACCTGGATAGTGGAAAGAGAGCACTGGCCGCACAAGATGT
CCTCTGACCTCCATCTATGCACAGTGCCAAGCAGTGGACTCGCATACAAAAATTAACTTAAAATGGTTTATCCCCTTTTCCTTAAAGGCCTTTT
GTCCTTTGATGGCATATACTATTACAGTACACCTTAAAAGTCTGTTGGGTTTTCTGACATGCATCTAACAAGAAAAATCAGGGCAGGAATTACT
TTTTTGATATGCTCTTTAAATTAAATATGTGCATTAAGCATGAGTGTAACTATGAGGGGTGATTTACACCACTGGAAATACCACCAATTACACC
AACACTCATCTTTGCCAAATTATCCAACATTGTTGAATACAGATTGTACAAGGAATACATCTCCTTATTTTTGTCATAATAAAAATACTGATTT
TGTAGACAATAGACCTTTTAGGATAACCCAGATGCCATTTTGATGGCATCTTAAGAAGGTACATCTGTGGGTATCAGAAAAAAAAACAAAAAAA
AACAAAAAAAAAAACACATATGCCTTGCAAAACACTTTTCTTGGTGGAGGTTTTTTTCTTGAAGGAGTGCATACTATTTTAGGATTTCCTCCTC
TGGGAATTAAAAAAGGGCTGTGATGGGTAGCAAATAGTCAAAAAAAAAAAAAATTACTTGCTTTTTACCCACAAAACATTCTTGCCTGCAGTTG
ATCTTAGAGACCCAGTCAGCTGTCTCCATAGGGTACTCTTTCCAGTAAATGTCTAATTCTTGACACCCTCGGCCTCCTTCCAGCCTAAATACTC
TGAATGTGTGACAAGGTAAATGGAAATATACCACCCAGCACTACTGTGCTCTCAATCAAGAGCTGGCTCTAAGTTGGCCTCCTCTGTTACTAGC
CTGAGGGAAATCAGTTTATCATCGTATTAAAGGTGAGTTTTCACTAGAGTCCCAATGCTCAGATCTTGGGAAAAGGGTGGTTTGTGTAACTAAG
TTGTTCTATATTCTAAGTATTATCTGCATGTGTTCGCTCTTTGAGACAAGGACTTGGGTAGCTGGAGCTGTCCTGGAATTCTTTTTTTTTTTTT
TTTTCCCTTGGTTTTTCGAGACAGGGTTTCTCTGTGTAGCCCTGGCTGTCCTGGAACTCACTTTGTAGACCAGGCTGGCCTCGAACTCAGAAAT
CCGCCTGCCTCTGCCTCCCAAGTGCTGGGACTACAGGCGTGCGCCACCACCGCCCAGCTTGTCCTGGAATTCTTAAGCATCAGCGGATGATCTT
GAACTCCTGATCTTCCCTGAAGGCACCTCCCAAGTACTGAGATAACAAACGGGGACCACCAAAGGGGCTTTCGATTACGTTTCACCACCGGTGA
CCACCTTCCCACCTTAGTCTCTCGAACATCGTGCTGCTCTTACTAGAAGTGGCATTTGCATTAGGGAGAACTCGGAATGTGTGTTAGAATCCAG
AGGGCTCAGGACACACATCCGTGGTTTATAATCACACACTCACCGGGCACTAACACAAGCCCTCTATAAAGCTCACAAGGTTCCCAAAGCACAG
CAAGAGGAGGACAATGAGACCCAGAAAGAAAGATCAGGAGAGGGACTTGAGATAGGTCCAGCAGCCCACTCCCCTGTGCCACCCCTTTCTCTTC
TTGGTCTTTTAGGCCCAATTGCTTCAAGTAGCACAGACACACGGGAGACAATGGGTAAGAAAGAGAGGGGGAAGGAGAGCACATAGCGAACCCA
TCTATCCTTTGGATATTGTGGAAAACAAACATTTAGAAAGTGAAGTCAATCCCATCCTGTATTCACCATGATCGCCCTATACAGAAATCACACA
AAGTCTCTTATTGAGGTAATTATGGGCCACTGCCTTAGCGTTCCACAGAAAATACTTGTTTGGCCTACCCAGGATTTATAGGGGAAGTGAAATC
ATTAAATAACTAGTTAACCTCTGTTTGGCAAAAAGAGTAAGTAATTACTGAAAAGGGGAGAAAGAAAAAAAAAAAAAAACCCTGCCTTTCACAA
ACAGCACAGATAGCATCCTGAAGGGATTTACACAATACACCAACGTTTGTTCTGTACCCAGAGTTGTTAAAATAATTGTGATGACAGGTGTCTA
GGTACCACTTTCTTAATGGTTTTCAATATCCATTGATTTATTATTGTGGCTCCCCCCACTTACATCTCTAGCTCTAAGAAGGGGCTAAGAAAAG
AGATTTTGCACCAAAGGCTGATTCTTTATTCCTGGGCTGAAAAGAACTAATAAAAATAATCACTCGGCTTTTAAGCAGAAAGCTTCCATTTACA
GTATTTCACCCTTACCCCCCCTCCAAGCCCCTTGTTATCTAACATAATGGCTAGAATACATTTCCGTCTCTCCCCTTAATAACTTCATGCATTA
ACATCCTCTTCACACATGCGCGTGCGTGCGCGCGCGCGCGCGCGCACACACACACACACACACACACAGAGGCAAACGCGCGCGCACCCAGCAA
AAGGCGGAGGTGGGGGGGAAAGAGGCAGCAGAAATCTCAGCTGTACTTCTAGCCCTTTTAAAAAGTTTGCTCTGTAAATTGGAATGAGGTCAGA
TTTGGAGCTTCTCATTGCACGCGGAGATTATTATTGCATCGGGTTCCAAGCCAATGGGAAGGACGGGGGAGGGGCTTGGCACGAGGAAGCGTTA
GTTACAGCGGCTGNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNCAGTGGGGGAACTCTGCCGGTAACCAGCTCCCCTTCTTGCAGGAGGGAGGGAGAAACA
TACATTTATTCATGCCGGTCTGTTGCATGCAAGCTTCTTGGCTTCCTACCTTGCAACAAAATAATTGCACCAACTCCTCAGCGCCGATTCCGCC
CACAGAGAGTCCCGGAGCCAGAGTCGCTTTGGCTTTGCACTGCAGGAAAGGGACTTAGGCGCTAGAGACGATGTCGCTTTCCTGAGCTACCGCG
AGCTCTCGTGAACTGCAATCGACTGCTTCAGGGAAAGGGGTGGGGGAAAGACTTGCCCCGGAGGCGGCGAGAAACTTGCGTTTGGAAGATACTC
CGGCTACCAACGTTTGGAGAAACTCCTCGCCGCGGCCGACTCCAGCCTCGGTGCCCGCAGGGAGCACTTCAGCGGTGAGGGAGGACGGAGGGCC
TCGGGGACTCTAGGTTGGCGGCGGGAGGCGGCCGCCCCTGGCCCGCGCGCCGCTCAGGGGACCCTGGTCTCCGCCCCGGGGAGCCCGCAGGGCC
CGGCGACCGCGCCGCGAGCGTGTGAGCGCGCGTGGGCGCCCGGCAAGCCGGGGCCATGGTACAACAGACCAACAACGCGGAGAACACTGAGGCT
CTGCTGGCCGGGGAGAGCTCGGACTCGGGCGCCGGCCTGGAGCTGGGCATCGCGTCCTCCCCGACGCCTGGCTCCACCGCGTCGACGGGCGGCA
AGGCGGACGACCCCAGCTGGTGCAAGACGCCCAGTGGCCACATCAAGCGGCCCATGAACGCCTTTATGGTGTGGTCGCAGATCGAGCGGCGCAA
GATCATGGAGCAGTCGCCCGACATGCACAACGCCGAGATCTCCAAGCGGCTAGGCAAACGCTGGAAGCTGCTCAAGGACAGCGACAAGATTCCG
TTCATCCAGGAGGCGGAGCGGCTGCGCCTCAAGCACATGGCTGACTACCCTGACTACAAGTACCGGCCGCGAAAGAAGGTGAAGTCGGGCAACG
CGGGCGCGGGATCGGCGGCCACAGCCAAGCCAGGGGAGAAGGGCGACAAGGTCGCGGGCAGCAGCGGCCACGCGGGAAGCAGCCACGCGGGGGG
TGGCGCGGGCGGCAGCTCCAAGCCCGCGCCCAAGAAGAGCTGTGGCCCCAAGGTGGCGGGCAGCTCGGTCGGCAAGCCCCACGCTAAGCTGGTC
CCGGCGGGCGGCAGCAAGGCGGCTGCATCGTTCTCTCCAGAGCAAGCTGCCCTGCTGCCCCTGGGGGAGCCCACGGCCGTCTACAAGGTGCGGA
CTCCCAGTGCGGCCACTCCGGCCGCCTCCTCCTCGCCGTCCAGTGCGCTGGCCACCCCAGCCAAACACCCTGCCGACAAGAAAGTGAAGCGCGT
CTACCTGTTTGGAAGCCTGGGCGCTTCGGCGTCTCCCGTCGGGGGCCTGGGAGCGAGCGCCGACCCCAGTGATCCACTGGGGTTGTACGAAGAT
GGAGGCCCGGGATGCTCGCCCGATGGCCGGAGTCTGAGCGGCCGCAGCAGCGCAGCATCATCGCCAGCCGCCAGCCGATCGCCCGCTGACCACC
GCGGCTACGCCAGCCTACGCGCAGCCTCGCCCGCCCCGTCCAGCGCGCCCTCGCACGCGTCCTCCTCGCTCTCCTCGTCCTCTTCCTCCTCCTC
GGGCTCTTCGTCGTCCGACGACGAGTTCGAAGACGACCTGCTCGACCTGAACCCCAGCTCAAACTTTGAGAGCATGTCCCTGGGCAGTTTCAGC
TCCTCATCGGCGCTCGATCGGGACCTGGATTTTAACTTCGAACCCGGCTCAGGCTCCCACTTCGAATTCCCGGACTATTGCACGCCCGAGGTGA
GCGAGATGATCTCGGGAGATTGGCTGGAGTCCAGCATCTCTAACCTGGTCTTCACCTACTGAAGGGAGCGCGGGCCGGGGAGAAGGTGGGCCAA
GAGGCAGGAGAGGAGAGAGGGAAAAAAGAAAACAAAAAACAAACAAACAAAAAAACAAACAAAAAAATGTTAAAAAGAAGAAGAAAGAAAGAAA
CAGAAAAAAAATGAGTTAAAAAGAATAGATGGAGAGTAGAAGGAGAAAGGGGAAAAGAAAAGGAAGAAAAAGTGCATAGAGCTGGCCTGGAACA
CGTCCCGCCGTTGGAGAAGGAGCGCGGGGGCGTTAGTGGACCCGCGCTCCCATCCCCCACTCTCAGGGCTGGGGACTCGAAGGAGGCGGAGAGT
AGACGGGGGCGACCTTTTTTTGTTGTTGTTATTGATGTTGGTGGTGGCTAAAAAAGCTACTTCGAGTTTCCTCCCCAATTTTGCTTGAAGAGAC
TCCCCCCCACCCCACCCTTCCAACGAGCTTCCGGACTTGGTTGCACCCCCAGCAAGAAAAGAAGCCAAGCAAAGCTTCTAAAGACCGAAGGAAT
CTTTCCCTCACCCCCACCCCCTCGCCTTGGTGATTTCTTGTTTGATTTTGCCTCATGGTCAAGAAAGGAGGGGGAAAATCCAGCGTGCCCCATC
TCCTACCCACCCCCTCTTTGTATTCTCTTTGTATTTTTCCCCTTCTTAAAATTTCTTTTTCTGCAATGAAGACAGAAAGAAGGCTCTGGGGTGA
TGCGTTTGGCATTTGTGTTGAGCTTAGGGGAGCATTGGCATGGAGAAACTCCACGCTGGCGAAGTCCCGGGCTGGGCTTTCTCCTCCTCCCCCC
ACCTTTTTTCCCCTTGTCCTTGCAGCTGGAGATGTGCTGGGAGTAGCAGGCCAGCCTCGGAAATGGACATGGGGACCTCGTGGAAGCCACAGCA
ACCTGGTTGGGGATGCAGAAGGACCCGGAGCACAGAGGGCGTTGGGGTTCCCGGGCCTCCGCCTGGGGTCTGTGCAAAAATAAAGAATTAAAAA
AAAAAAAAAAATCAGAGAGGTGAGGCTACCCAGAGCCGGCGGGAGGACAGGAGACTGTGGGATGTGGTCCAGGGGCCGCGGAGTGGTTTGGGGG
GGAAAAGAGATTTTTTTCTTCTCTTAATGGGAATCGTGATGGTGTTGAGTTATTTCACTGGTGGGGTTAATATAGCATGTTATCCTGTCTATCT
TTTGAAGATTTCTGTATAAGACTGTTGAGCAGTTTTTAAAATAGTGTAGGATAATATAAAAAGCGGATAGATGGCGCTATGTTTGATTCCTACA
ACGAACCGATCACCAGCTCCTTTTCATTTTTAACTCTTTAAAAGGATTCAAACGCAACTCAAATCTGTGCTGGACTTTTGAAAAACCACAATTC
AGGACCAAATTTTTTCTCCGTGTGTGTTTATTCCTTATAGGTGTAATTGAGAAGACCCGTTTATTTTTACCCCCTCACGGACTCTCCATCTTTG
TATTTTTTTTTCCCTTGTAAATGTAATCAGATGCCATTTTATATGTGGACGTATTTATACTGGCCAAACAGTTTTCTTATTTTGTCCCCCCTTT
TTTTCCCTTTTCATTTGCTTTCTTGTCTTTTAACCTCGTTTCTTTATCTTCTTTTTTATTTTCTTTCTTTTTCTTCGTTTTTTTTTTTTTCTAG
TGTTGTTACCCACGCCATTTTACGTCTCCCTTCACTGAAGGGCTAGAGTTTTAACTTTTAATTTTTTATATTTAAATGTAGACTTTTGACACTT
TTAAAAAACAAAAAATGACAAGAGAGATGAAAACGTTTGATTATTTTCTCAGTGTATTTTTGTAAAAAATATATAAAGGGGGTGTTAATCGGTG
TAAATGCCTGTTTGGATTTCCTGATTTTATAATAGGGTGGCTGGTTAATATCTCACACAGTTTGAAAAATCAGCCCCTGGTTTCTCCATGTTTA
CACTTCAATCTGCAGGCTTCTTAAAGTGACAGTATCCCTTAACCTGCCACCGGTTTCCACCTTTCAACCCCCTGGTCTTATAAGGGGAGGAGAG
TTCAGCCCAGCACCATAATGCTTTAAAAAAAAAAATTTTTTTTTTTTAACGAATTGCTGTTCTGTCCAGAGGTTTTTAAAACTGGTGCATTCAC
AGCAAAAAGGGATTCTGTAGCTTTAACTTGTAAACCACATCTTTTTTGCACTTTTTTTTATAAAGCAAAAACGTGCCGTTTAAACCACTGGATC
TATCTAAATGCCGATTTGAGTTCGCGACACAATGTACTGCGTTTTCATTCTTGTATTTGACTATTTAATCCTTTCTACTTGTCGCTAAATATAA
TTGTTTCAGTCCTATGGCATGGTGATAGCATATGTGTTCAGGTTTATAGCTGTTGTGTTTAAAGATTGAAAAAAGTGGAAAACATCTTTGTACA
TTTAAGTCTGTATTATAATAAGCAAAAAGATTGTGTGTATGTATGTTTAATATAACATGACAGGCACGAGGACGCCTGCCTTTTAAGAGGCAGT
TCCGTTAAGGGTTTTTGTTTTAAAACTCTTTTTTTTCCTTTCTTTTTTTATTTCTTTTTTTTTAACCATCCATCCTGTGCAATATGCCGTGTAG
AATATTTGTCTTAAAATTCAAGGCCACAAAAAAAAGCAAAAAAAAAAAAACAAAAAAAAAAACAAAAAACAATATTTGGGGGAAGAGAAAAAGA
AAAAAAATCATGCCAGCTAATCCTGTCCGTCACTGCCTGTCAGGTTGTTCCTATATACCTTCTGTAAATAACTTTTTTTGAGAAGGAAATAAAA
TCAGCTGGAACTGAACCCTAAATCTTGACTTTTGTCATCCTTATTCCTGGGATCTCTCCTTTCACTGCAGGCTGCTGCTTTGCTGCTTTGCAGC
TTCGCGGTGGCATGGAAGCTGGTGCTGATTCAGTTAAGCCATCTTAATGGCAAACCCTAAGTTCCTCTTGGCCAGATTTCGCAGGCCTGACCCC
GAGTGATGGCATTGGCCACGTGTGTGTCAACCCTCCCTCCGCACGCCAGCCTTCGAGGAATTCTCTGCGTCTACTGTACATCAGCAGGCACCCT
TTGTCTGGAAATTGCAAAGACGGGGCAGCGGCAGGCTTCTCTGCTCTCAATCCAGCCGGCAGGCTGAACTTTTTTAAATGTATTTTTTGCTTTT
GTGTCTCTAAGCCACCATCCCCTTTACACACCAAGATTTGTTTTGCACAGACCAAGAAGAACAAATGAGCCTGTTAAATTATTAGGTCTTCTAG
GAGAAAAGCCTTCCATAGTTACAAGATGGGAAAACCAAAATCAACTTAGGCTTGCTTTGTTGGGGTGGGGTGGAGGAGTCCCCTTAAATGCAAA
GGTCAGTAGGGTGTGGTGTAATCCAAGTACTTGAGAGGCAGGCCAGGCTCAAGGATCCTGAGTTTAAGGCCTGAGTGGACTACTTGATTGAAAC
TCTGTTTCCAGAACTACCAAAGTTACTATGGCCCTAAGCTGTTCATCTTATGCCCCGCCCAACATGGGCGTCACTGTGAGTAAAATATAGCCTA
CCTGCTCACCACTGGAATGACAGGCCAAGTAGGAGAGTGTAGATTTTTATAGGCACCAGACACAGCATTAGTGAAGCCTGCATGCAGCTGGACA
GTAGTCGGTGTTTATTTTAGAGATCAACGGTCTCCATGGGCAAATAGAAGTATCTTCCCTTCCCAAGGCTCATCTTCACAAATGTGTGCTTTCC
ATGTTTCCTAGTCTTTCCGCCCTGGAGAGAAAGGGTTAACATGCAGCACTTGGAAACAATCCTTTCTGAGGATCGGAGGCAAGGTGCTTGCATC
CTGGGATCACAACAGAAAGAAGACTTAAGGGCCAGTACCGCAAGCATTTTTGCACAGTAAAAAAGCCATTTGCTAGGGGTTGGCTGTGATCAAC
TTACACTACATACTTGGATGAAGTATTCAAGGGGGGGAAAAACTGAAAAAGCAATTCCCTCTCTAAATACTGTTCTAATATCATTTTTCCCTGA
GACCTTGAAACAGTATGGTGTAAATTTTTCTTTTTTAAAAGAGCTTTGGGAAAGAGTTGATATGCTTGTTTAATGGTTGATTATTATGGTGAGG
ATAATAGAGCATCTTAGGGTCTTGGAGAAGAAAGCCTTTGATAGTTACAAGACGGGGTGGGGGTGGGGATGACTTAGGCTTGCTTTGTTAGATT
TGGGGTGGGGGTTTCCCCTCAAATGCAAAGGTCAGTAGGGTGTGGTGTAATCGAAACACTTAGAGACATGCCAGGCTAAAGGATTCTGACTTTA
AGGCCATAGTGGACTACTTGAGACCCTGTTTCCAGAACAACAAAAGACCTTGTTCCTTCCCCCTTCTCCCCCGACCAGACAAAAACATCGAAAT
CCTACTGTAAATGAATCCTGTATGAATCATTTTTAAGTGATGCATATTTTCAAATAAGTTTGTATTTTTGTAGGCTCTTCTGCTACAGCCTTGT
TTATTTTCCCCTGACTTTGCATGGGAGGAGCAAGCAGATGCTTTCAAATATTTTCTTTAAGAATCCTAAGGCTCAGGGTGAAAGTGGAGCAGTG
CACTTAGCTAGCGTGCTAGAGGCCCTGGGTTCTGTTCCCAGGCCCCTGAAAAAAACAAGGAATTCTGGCACATCACCCTTTTCTCTTTAAAGTG
TTGATACCGCGTGCTTCCTAGCAGGGGCGTGGCAGATGTGAACCACTGGAACCCTGTATTCAGGAAGGAGACTTACATGCCAGACTGTGCAAGG
GAAAATAGATATTAGAAAACCACTTCACGGTGACTTTCAGATGGTGTGGCTCAGCAGAAAAGAGTGAGCTCCAGCTAGCTTTTCGCTCTTGTTC
TTAAGACCCGGCTTCAGGCCCTGTGCTCAGTCAGCAATGTGGATTAGTGACACTTGTCACCATTGTCAGTTCTTAGCTGAATCCCCTGAAGAGC
CATGAAAATAAATAAATAAATAAATAGGCAGCCTCCAGGCTCCTCTGGTGGGGTGAGTGCCTGCAGGGGCCTCAGGTTACTGCTGACTCTAGTC
TGAGTAGGTGTCCTAAGGAAAGAACAGAGTTGGAAGGCCAGCAACCACTCGGTTTATCTGCTGTATAGATGGGATCAGAGTGCTTCATCATAAA
TCAATCGTTTGGTTGGTTGGTTTTGGGTTTTCCGAGGCAGGGTTTCTCTGTAAGGCCCTGGCCTAGAACTCTCTCAGTAGAGGGGGAGCGCCTT
GAACGCAAGGATCTTCCCGTCTCTGGAAGTGAGTGCTGAGCCTGAAGATGCGCACCCAACACCACACCCTCTCACCTCGGATCTTAACCCACTG
ACCTAGAAGGTTTTGGATAGCAGGGCATGGCGGTGCTGTCCATTTAAAAAAGAAAACTTCCAGAAAAATCTAAGACCCCCCAAATCTGTTAGAT
TTCTCAAGGTGGAAGTTTTCTGTCATTTTTTTTTTTAATGGATACTTTGAGTTGTTTTTTTTTTTGTTTTTTTTGTTTTTTTTTGTTTTTTTTT
TTTTTAAAGATTTATTTATTTATTATATGTAAGTACACTGTAGCTGTCTTCAGACACACCAGAAGAGGGAGTCAGATCTTGTTAGGGATGGTTG
TGAGCCACCATGTGGTTGCTGGGATTTGAACTCTGGACCTTCGGAAGAGCAGTCGGGTGCTCTTACCTGCTGAGCCATCTCACCAGCCCTTGAG
TTGTTTTTAAGATTGATTTTATGGAAGTGTTTGCGAGTGGTGTGCACATGTGCATACAAGGAGCCTCACAACAGCAAGGTGTCCTGTTGCCTGG
AGCTGCAGTCACAGGCAGGTGAGCCACCTAACTCCATACCAGGAACTAAGTTCAGGTTCTCTGGAAGAACGGGAAGTAGTATCAACTGCCAGAG
CCTCTCCAGCTTTCCTGTTGATTGTGGTTCACAGCCCTGGCCCCAGGACTATAAATACACCTCTGGCTTCTGATCTCTTGTGTTTCCCCCACAC
ACACACACACACACCCCACCCCCGGCCTCTCTGATTCATCCTAGAAAACAACCTGAAAATCCATGTCGGTTTTTTTTTTTCACCCTGCCCACCA
TTTGAAAGCTGGGGTGGGCAGCTTGTCTGCATTTTCACCCCACCCACCATTTGGAAGCTGAGTTGGGCAGATGGGGGCTAGGGGTGTGTGTGAG
CATCTTTTGATATCTGCATGGTGTTTCAGACCACACTAACCATCATTATTATCCTTTGTCCAGGGTGAGGTACTCGGTTCTCGTGCAGCTTGTC
AAGACTCTGCACAAAATGGTGGTGGCTCTCCTCCTCCAATCTCCACCACCCACCACTGCAGCACGAAGCCTTTCCTGGGGAAGCTCTCCGGATG
GTGGAGCCCAGGAGGCAGAGGCAGGAGGGTCTCTGTGAGTTCACAACCAGTCTGCATAATGAGTTCCAGAACAGCTCAGACTACATAGAACAGG
ATTAACCCTTTAATGCCTGTTTCTCCACACTTACTTTGTAGTGAGTTCTTGCATAAAACCTGTTCTTCAAGCTTCCAAAATATATTCATTTCCC
AAAATATTTGCATTTTGTGCTATCTTTGGCAAGCAGCGTTGAATTTAGTCAAAATAATCCATGTGTGAATACTGGCTCTGAGCTAGGCTGGCTG
GAGCACGGTGGTCAAGTTTCTTAGTCCCTTGGAGCCTCCGTTCTAAATCGTTCCATAGAAGGCCACCTGACTGAGAAAGCTGTTGTGAGAATTG
AGTGTGACCCCATATGTGGAACACATGCAGCAATGCCTGGCAGCAATCTGTGCTCATTAGGTACCAGCCCTTCACCTTGTGCGGCTGATTATAA
AAAGGTAAAGGTTGTGCAAGCCAACCACAGTGCCAGCCACTCAGAAATCAGACAGCACTAAATAACAATAAATATGCTGTCTGCTTCTCAAATC
CTTAATATTAGTATGAATTGAGGACAAGGCTAGAGCTGGAGCCCAGTGGCTAAGTGCTCGTGAAGCACACATGAGGCCATGAGGCCCTGGATTG
GCCTCCCAGTATCAATCACAATAAGACAGACCTGGTGAAGCCATGCCAGTACTGGGGAAGATAGCTCAAGGCTATCTTTGGTTAAATAGTAGGT
CTGGGGGCAGCCTGGACTTTGTGAGCTCTTGTCTGAAAACAAAAAGAATGGTTTGGGAGCGTGTGTGTGTGTGTGTGTGTGTGTGTGTGTGTGT
GTGTGTGTATTATTGGGAATTGAACCTTGAGTCTGGGACATGCTAGGTTCTCTTCCATGGATCTAAGTCCCAGCCAGAATTCTTTTTATTTTTT
TAATATATTTTTAAATTATTGCTAGTGAATTTCATGCAAGTATACAATGCAGGTTTTGATCAAATCCATCCTCTACACTCCCCCCGCCCCCCGC
CCCCTAGCCAGGACTCTTTAAAAGAGCAGAACCAGACCACAGTCTTAAGTTCTCCATATATTCTCAGTGCAAACCAAAGTGAAAATGACTACCC
TCACAGTGTGTTAGCATGATCTCAATAAGTATACAGGCAAAAGATTGATACCACCCTTTTTACAAGGAGATGTGGCGATGATTCAGTAGTAAAC
CTGAGATCATAGAATCAGAAGGGGAAGAAGGGTGGGGCTGTGGCTCAGTGGTAGGATTCTTGCCCTAACATGTATGAGACCCTGGGTTGGAGCC
CCAGCACTCAACTGCATTAATTAGGAAAGAAGAGGAAGGGGGCCCTGGAATCTTATTTCCTGAGCCCAAGAATGAACTTAGATCCAGTGTCTCT
CCAAGTGTGGTGGCACATCATTCAGAAGACAAAAACAGGAGGATTGCTTGGCGACCAAGTTTAAGGCCAGGCTGGCCTACAAAGTGTATTCCGG
GATAGTTAGAACTGTTACATAATGAGACTCTCTGTCTCTAGAAAACAAAAGCAAGCCAGGAGCTGGTGGGCCGTGCATGCCTTTCATCCCTGCA
CTTAGGAAGCAGAGACAGGTAAATCTCTGAGTTCCAAGCCAGCCTGGTTTCCAGAGTGAGTGCTAGACCAGCCAAGGCTACACAGAGAAACGCT
GTCTTGAAAAACAAAACAAAACAAAACAAAAAAGAAAGAAAAAAAAAGAGAGAGAGAAAAAGGAAGGAAAGGAAATGAAACAAAAATGAAAATT
AATTAAAACAACAACAACAACAAAAACAATCAGAATAGGTGTCCCCGTTTGAGAACTAGAGAAGCATAGGTGGGAGGCCTTGAGTCTTTGGTAA
GGATTGGCCATCACCTCAACCCTCCACATCCTTCCTTCAGCTCTGATCCATTTCTCCAAGGCTGTCTTGTACATCTCAGTTTGAAGTGCAAGCT
TAAGTAATTTGTGTGTGTGTGTGTGTGTGTGTGTGTGTGTGTGTGTGTGTGAATACATATTAGTTGAACAAATGTGTGTTAAAATATGAATGCA
CGGTCTTCATTTCAGGAGTCAGCAAGGCAGAAGAAATGATCTAGCTATCAGTAGTGGATCTTCTGCTCTGTCATTCAAAAGGTGGGGTGGGTGA
CCACGCTGCTGGTTATTCAGAATGCCTTCACACAGAGGCTCTAGGCTACCCATCCTATCCTAATGAGATTGGCCGTTGCTACAGTTAACTTGAA
GGGTTTTATGGTTTCCTGCATGTGTTGTTTTCCTGCTTAATGTTAAAAAGAAGAAGAAGAAAAAGCTCACGTCACTAAACTGAATTTCCCAGGG
GGCTTTTGTATCTTCTGCAGTCTGGCTTATGCCTTGGTTATGATGAGATACCTCTATGTGTGGATATCTGTAGATACAGGTGATAACAAAGTTA
GCCCATTTTCTTTGCTTCTGACAGTTTCCATTCAAGACAGTTGGTTTAAATGGAGTCCTGCCAACATGCCAGGCACAAGTCAAGTCGTGGCATG
AGCTTTGCAGCCAGAGCCTTTCCTTCAACCTGCAGACTTTGTGGTAAAGGCCAAAGTTACAACTGATTTATACACCACCTTGCTTTGAACCGCC
CCTAAGACACTCCAGTGACAAGACATCGACATCTGATTGGAAGGTTTTGGATTTTATTTTTAATGACTTGGAGAGAAGGAACAAATGAAGTATA
ATTTACTCAGCCCTCTTGCTTCTTTAGTTATTTTCAATACAATTATTTCTCTATTTCCTGCTCCAGGGTGGAGTTTGGAGGGAGGGAGGAAGGG
AAGGAGAGAGGGAGGAAGAGAGGGAAATGGAAGCCCTGTCTGTCAACTTTAGCAGGCTCATTCTGTAGTGAATGCACAGTATGTGGTGGTGGCA
TCCAAGTTAAGGAGCAGGCAATTGCTTTTCTAAGAGCCAAGCAGCCTGTGCCGGCTTAAAGGATCAATTAAGTCCTGTTGTCTGTGTGGTTAAT
TGTCACCTTCTTACCAGTGGCTGTCCTAACTCCCTCAGCTCTGGCACCTAAAACCTGTGAAAATAGTTTCCTGCAACTCTTCGTCCTCCCTCCC
ACGCCTCTCACTTTCTCACCCCCCTTTCCCACACTCCCTATGTGGGCCTTTTATCTCTCCTGTTTGCCATTCCCCGGAGAAGGCAGACTTTGAA
TAATTTATCTAAAGTTCCTGGGAAGTTGTGGGGCTGTAGTCTCAAAACACACAGCACTTTGTTCCTCCCCCAGATAGAAAGGCCAGAAGTCTGT
CTTTGACCTAGCCCCTCCTCAGTGAATCCAGGAAATGAAGTAGGTTACTAAAGCCATGGAAAGTCCTCTGTGTTTCTCAAATAGTTTAGCCAGA
GTATTCCCAAATCGTTTCTTTTAAGGTATTGGGGGGGGGGGGGGTCAAGGTACAATCCGAACACTCAGGATATGGAGGTAGAAGGGTTGAGTTC
AGGGCCAGCCTTTGCTATGTAGGGAATTTGAGGCTTAGCTGGGCTACAAGAAGACACTTTGTCTTTTTTTTTTTTTTTTTTTTTTTTTTTTTTT
TTTTTTTGGTTTTTCGAGACAGGGTTTCTCTGTATAGCTCTGGCTGTCCTGGACCTCACTTTTGTAGACCAGGCTGGCCTCGAACTCAGAAATC
CGCCTGCCTCTGCCTCCCGAGTGCTGGGATTAAAGGCGTGCGCCACCACGCCCGGCAACACTTTGTCTTAAAATGATGA
MOUSE SEQUENCE-mRNA
AGAGCAGCAGAGTGAGGGGAAGAGGGCCGTCTCCCTCCCGGTTTCCAGTTCTTGCACGCTGTTTCTTAGAGAGTCTGCAGTGGGGGAACTCTGC
CGGTAACCAGCTCCCCTTCTTGCAGGAGGGAGGGAGAAACATACATTTATTCATGCCGGTCTGTTGCATGCAAGCTTCTTGGCTTCCTACCTTG
CAACAAAATAATTGCACCAACTCCTCAGCGCCGATTCCGCCCACAGAGAGTCCCGGAGCCAGAGTCGCTTTGGCTTTGCACTGCAGGAAAGGGA
CTTAGGCGCTAGAGACGATGTCGCTTTCCTGAGCTACCGCGAGCTCTCGTGAACTGCAATCGACTGCTTCAGGGAAAGGGGTGGGGGAAAGACT
TGCCCCGGAGGCGGCGAGAAACTTGCGTTTGGAAGATACTCCGGCTACCAACGTTTGGAGAAACTCCTCGCCGCGGCCGACTCCAGCCTCGGTG
CCCGCAGGAGCACTTCAGCGGTGAGGGAGGACGGAGGGCCTCGGGGACTCTAGGTTGGCGGCGGGAGGCGGCCGCCCTGGCCCGCGCGCCGCTC
AGGGGACCCTGGTCTCCGCCCCGGGGAGCCCGCAGGCCCGGCGACCGCGCCGCGAGCGTGTGAGCGCGCGTGGGCGCCGGCAAGCCGGGGCCAT
GGTACAACAGACCAACAACGCGGAGAACACTGAGGCTCTGCTGGCCGGGGAGAGCTCGGACTCGGGCGCCGGCCTGGAGCTGGGCATCGCGTCC
TCCCCGACGCCTGGCTCCACCGCGTCGACGGGCGGCAAGGCGGACGACCCCAGCTGGTGCAAGACGCCCAGTGGCCACATCAAGCGGCCCATGA
ACGCCTTTATGGTGTGGTCGCAGATCGAGCGGCGCAAGATCATGGAGCAGTCGCCCGACATGCACAACGCCGAGATCTCCAAGCGGCTAGGCAA
ACGCTGGAAGCTGCTCAAGGACAGCGACAAGATTCCGTTCATCCAGGAGGCGGAGCGCCTGCGCCTCAAGCACATGGCTGACTACCCTGACTAC
AAGTACCGGCCGCGAAAGAAGGTGAAGTCGGGCAACGCGGGCGCGGGATCGGCGGCCACAGCCAAGCCAGGGGAGAAGGGCGACAAGGTCGCGG
GCAGCAGCGGCCACGCGGGAAGCAGCCACGCGGGGGGTGGCGCGGGCGGCACGTCCAAGCCCACTCCCAAGAAGAGCTGTGGCCCCAAGGTGGC
GGGCAGCTCGGTCGGCAAGCCCCACGCTAAGCTGGTCCCGGCGGGCGGCAGCAAGGCGGCTGCATCGTTCTCTCCAGAGCAAGCTGCCCTGCTG
CCCCTGGGGGAGCCCACGGCCGTCTACAAGGTGCGGACTCCCCAGCTGGCCACTCCGGCCGCCTCCTCCTCGCCGTCCAGTGCGCTGGCCACCC
CAGCCAAACACCCTGCCGACAAGAAAGTGAAGCACGTCTACCTGTTTGGAAGCCTGGGCGCTTCGGCGTCTCCCGTCGGGGGCCTGGGTGCGTG
CGCCGACCCCAGTGATCCACTGGGGTTGTACGAAGATGGAGGCCCGGGATGCTCGCCCGATGGCCGGAGTCTGAGCGGCCGCAGCAGCGCAGCA
TCATCGCCAGCCGCCAGCCGATCGCCCGCTGACCACCGCGGCTACGCCAGCCTACGCGCAGCCTCGCCCGCCCCGTCCAGCGCGCCCTCGCACG
CGTCCTCCTCGCTCTCCTCGTCCTCTTCCTCCTCCTCGGGCTCTTCGTCGTCCGACGACGAGTTCGAAGACGACCTGCTCGACCTGAACCCCAG
CTCAAACTTTGAGAGCATGTCCCTGGGCAGTTTCAGCTCCTCATCGGCGCTCGATCGGGACCTGGATTTTAACTTCGAACCCGGCTCAGGCTCC
CACTTCGAATTCCCGGACTATTGCACGCCCGAGGTGAGCGAGATGATCTCGGGAGATTGGCTGGAGTCCAGCATCTCTAACCTGGTCTTCACCT
ACTGAAGGGAGCGCGGGCCGGGGAGAAGGTGGGCCAAGAGGCAGGAGAGGAGAGAGGGAAAAAAGAAACAAAAAACAAACAAAAAAAAAAAAAG
A
MOUSE SEQUENCE-CODING
ATGGTACAACAGACCAACAACGCGGAGAACACTGAGGCTCTGCTGGCCGGGGAGAGCTCGGACTCGGGCGCCGGCCTGGAGCTGGGCATCGCGT
CCTCCCCGACGCCTGGCTCCACCGCGTCGACGGGCGGCAAGGCGGACGACCCCAGCTGGTGCAAGACGCCCAGTGGCCACATCAAGCGGCCCAT
GAACGCCTTTATGGTGTGGTCGCAGATCGAGCGGCGCAAGATCATGGAGCAGTCGCCCGACATGCACAACGCCGAGATCTCCAAGCGGCTAGGC
AAACGCTGGAAGCTGCTCAAGGACAGCGACAAGATTCCGTTCATCCAGGAGGCGGAGCGCCTGCGCCTCAAGCACATGGCTGACTACCCTGACT
ACAAGTACCGGCCGCGAAAGAAGGTGAAGTCGGGCAACGCGGGCGCGGGATCGGCGGCCACAGCCAAGCCAGGGGAGAAGGGCGACAAGGTCGC
GGGCAGCAGCGGCCACGCGGGAAGCAGCCACGCGGGGGGTGGCGCGGGCGGCACGTCCAAGCCCACTCCCAAGAAGAGCTGTGGCCCCAAGGTG
GCGGGCAGCTCGGTCGGCAAGCCCCACGCTAAGCTGGTCCCGGCGGGCGGCAGCAAGGCGGCTGCATCGTTCTCTCCAGAGCAAGCTGCCCTGC
TGCCCCTGGGGGAGCCCACGGCCGTCTACAAGGTGCGGACTCCCCAGCTGGCCACTCCGGCCGCCTCCTCCTCGCCGTCCAGTGCGCTGGCCAC
CCCAGCCAAACACCCTGCCGACAAGAAAGTGAAGCACGTCTACCTGTTTGGAAGCCTGGGCGCTTCGGCGTCTCCCGTCGGGGGCCTGGGTGCG
TGCGCCGACCCCAGTGATCCACTGGGGTTGTACGAAGATGGAGGCCCGGGATGCTCGCCCGATGGCCGGAGTCTGAGCGGCCGCAGCAGCGCAG
CATCATCGCCAGCCGCCAGCCGATCGCCCGCTGACCACCGCGGCTACGCCAGCCTACGCGCAGCCTCGCCCGCCCCGTCCAGCGCGCCCTCGCA
CGCGTCCTCCTCGCTCTCCTCGTCCTCTTCCTCCTCCTCGGGCTCTTCGTCGTCCGACGACGAGTTCGAAGACGACCTGCTCGACCTGAACCCC
AGCTCAAACTTTGAGAGCATGTCCCTGGGCAGTTTCAGCTCCTCATCGGCGCTCGATCGGGACCTGGATTTTAACTTCGAACCCGGCTCAGGCT
CCCACTTCGAATTCCCGGACTATTGCACGCCCGAGGTGAGCGAGATGATCTCGGGAGATTGGCTGGAGTCCAGCATCTCTAACCTGGTCTTCAC
CTACTGA
HUMAN SEQUENCE-GENOMIC
TATGAAGTTTCCTCTTAGCACTGCTTTTGCTGTTATCTCAGAGGTTTTAATAGATTGTATTACTATTGTCATTCAGTTAGAAGAATTTTTTAAT
TTCCATCTTGATTTTATTTTTGACCCAGTGATCATTCAGCAGCAGGTTACTTAATTTCCATGTATTTGCATGGCTTTGAAGGTTCCTTTCGGAG
TTGATTTCTAGTTTTATTCCACTGTGGTCTGAGAGAGTGCTTGATATAATTTCAATTTTCTTAAATTTATTGAGGCTTGTTTTGTGGCCTATCA
TATGATCTATCTTAGAGAAAGTTCCATGCACTGATGAATAGAATGAATATTCTGCTGTTGTTGGGTAGAATGTTCTGTAAATATCTGTTAAGTC
CGTTTGTTTCAGGGTATAGTTTAAATCCAGTGTTTCCTTGTTGACTTTCTGTCTTGATGACCTGTCTAGTGCTGTCAGTGGAGTACTGAAGTTC
CCCAGTATTCTCGTGTTACTTTCTATCTCATTTCTTAGGCCTACTAGTAATTATTTTATAATTTGGGAGTTCAAGTTAGGTGCATATATATATA
GAATTGTGATATTTTCTTGTTGGACAAGGTCTTTTATCATTATATAATGTCCCTCATTGTCTTTTTAAACTGCTGTTGTTTTAAAATTTGTTTT
GGCCGGGCACAGTGGCTCATGCCTGTAATCCCAGCACTTTGGGAGGCCAACACAGGTAAATCACCTGAGGTCAGGAGTTCAAGACCAGCCTGGC
CAACATGGGGAAACCCCGTCTCTACTAAAAATACAAAAATTAGCTAGGTGTGGTGGCAGATGTCTGTAATGCCAGCTACTGGGGAGGCTAAAGC
AGGAGAATCACTTGAACTTGGGAGGCGGAGGTTGCAGTGAGCTGAGTTCACACCATTGTACTCCAGCCTGGGCGATAGGAGTGAAAGTCTGTCT
CGAAAAATAATAATAAACAAAAAAATAAACTTTGTTTTACCTGATATAAGAATAGATACTCCGCCGCTGGGCGCAGTGGCTTACGCCTGTAATC
CCAGCACTTTGGGAGGTCAAGGCAGGTGAATCACCTGAGGTCAGAAGTTCAAGAGCCAGCCTGGCCAACATGGTGAAGCCCCGTCTCTACTAAA
AATACAAAAATTAGTCGGGCGTGGTGGTGGGTGCCTGTAACCCCCGCTACTCGGGAGGCTAAGGCAGGAGAATCGCTTGAACCCGCGAGGTGGA
GGTTGCAGTGAGCCAAGATCATGCCACTGCACTCCAGCCTGGACGACAGAGTGAGACTCCATCTCAAAAAAAATAAAATAAAAAAAAAAAGAAT
AGGTACTCCTGCTCGCTTTTGGTGTCTATTTGCATAGAATGTCTTTTTCCATGGGGTTATTCAATTTTAGCTAACTCCCATATACACTATGGGC
ATTGGTGAAAATACTGACTGAATTTTTAATAAAGATCTAGAGTCATTAAGAAAAAGTTACCTTCCTGCATGAACTTATATTTTGGCATCTATCA
AATGGCATTGTAATTAATACAACTCTGACCTCTATTACTGCATAACAAACTACCCAAAGCATAGTGATTTAACGCAACTTATTAGTTCTCATAA
TTCTGTGGGGTGTCAATCTGAGTGATTCTTCTGTTGTACCCATCTGGAATCGCTCCTGTGGCTGCAGTCATCTGACAAACCAACCAAAACTGGA
GAGTCCAAGATGGCCTCAGTCACATGCCTGGGATGCTGGCCTTGGAGGGGCACCTTGGTTTTCCCTATGGGGCCTTCAATCCTCGAGTAAGATC
AGCTGCTGAAGGTAGTTCACAACATGATGTTTTCATGGTCTCACTGGAGTAGGTTCACAACACGACGGTTTCAATGGACAAACCCCAGTACATG
GGCACTTGTGAAGTCTGTTTGTCTCATGCTTGCTAATGCCCCATTTCCCAAAGCAACTCAGTGACCAAATCCAGAATCAGAGAGAAAAAGGTCC
ACATAAAAGCACACGTACTAGGAGGACGATTTATTACAAGAATCTACCACATCCACTGTAAATTTAAGAACTCAGAATACAAGTAGCATCCTCA
TTATTTAGCGTTTGCCTGCCACCCATAGTGCATAATTCACACCTAGGCAGTAGTACTCCTAGGGAGAATTTTTAAATTTTTGGACAACATTTTG
GGTGTTCTAGTGAAATACAGCCCTACCATGCATATTTGAAATGTATATTATCTTATTATAAATTACCTACTGTGTATTTCTCCTTTAACATTGT
AGTTTGAACATTATGGTTTCTTTTTTAATTTTGTGCATAGTAAGTTACGTAATCCACAGATTTTATTTCAGGAGAGTAAAGTTATATTACAAAA
TATTTGTTACAATAAGGCAGCGGTTTGGGTCTGATATTTTTGAAATTAATGAGTTAAGTTTGAGTGTTTTAAGTACTAGCCTTCAGTTATTTTT
GAATCGTGATAGCCAGATAAAGTGTCACTAAATTATTTTTTTAACTTGGAATGTGGGACAGGAAGGATGGAGTTTCATGCTTCAGGAAGCAGTG
AAAAATCACAAAACCTCTGTACTGCTAGACCCAACACTTTTCCAGATCCCCAAACTATCTCTATCACCCTCCTTTCATGGCAAATTTGCATATG
CATTATTGTCAGAATTTTGAACTTATTTTACAACCATATACACAGAATTCTTTTCTAGCTTGCTGCTTAACTGAACAGATGGTGCAGTTTCCCA
GCCCAGCAGCTGGAGGGGACCGAGGATGCGCTCGGCACACAGTAGGAGCACCATAAACGTTGATTGACTGAGTTCATTGAGGCGGTTGACGCCA
GGCCATCCACTCCCACGCGGTACTCTTATCTTTCTCCAGGCCCACACCCCTGGCCCGAGAGGCGGTAGCTGGGAGCAGATGGCGAGTCCCGGGT
GCCAGGTGTACTGCACTCCAGGCGCGGTCTGGGGCACTGGGTTCTACGGTCCCTGCCCCACCCCTCCCATCCCTATGAGTTCCCCCAAAGCTGT
CTGCGCCAAGTGTGACGTCTGACTACGGCGGGAACATGGGAGGGGAAGGGCTGGAAGAAAGGAGATCGGTTGCCTGGCGAGGGGGCCCGTGCGT
CCTACAGTCGCTACCCGCCACGCACGCGTCCCTCTCCTCCTGACCCGCGTCACTTAGCAGCCAATGCGGTCTCCGTGCCTTCCCACTTCCCCGC
AATCAAGCGCCTTTCTTCCTCCGCCTCCCCTCTTTCTGTTATCTGACATTTTGCTCTGCTTCCCTCTGCCCACTCCCCAGCGGGATTCCCTCCC
TGGCAGACGCGCGCGCCCCAGCCCGCCCTCGCACAACCTCCAATAGCAGGTCAACTGGCTTGTAACGCTGGCTTAGGGTTGCTCTTGAAGTGTT
TTTCCAAAAGCGAAATCACCCGCTTTCCCCCACGGGGCTAAAACTGTAATTGCAACAACGGGCTGTTGCGTTGGGGGTGGGAAGCCCGTCGGGC
GTGCCCCACCTAGACCGCCTGCTGGGGCCACTGGGGTGGTTTTGCCGCCCCCTCAACCCCTGCCACCCCGGGTCGAGTAGGCAGCTCCACCCGG
ACGCTTGCGCCAGGGCCTGACTGCCGCGGCTGCCAGCGGAAAGGCAGAGACCAGCTGATCCAAGGAGGACAAAGCGAGCCGAGACCTTCCGCAG
AGCTTGGCTTCCTCGTAACTGCGCCCAGGGCGCAGCCTCTCCCTGTTAACTACCTGCCATATGTTCCCTTCTCTCAGACCCGAAACCGCCTCCT
ACAGGTGCTGGGGACTGAATCTGGAGCTTCGGGCGCGGCCCTTGACAAGGGCGCCTGGCACCCTGGGGCGCCTCCGTGGCCCTCGCAAACTCAG
AGAGCCCCCACCGTCCAGTCTTTCCTGCACGAGCGAGGGATCGAGGGAAGGCGCCAATGCACGTCTGTCCCTCCGTGATCCGCGCAGAAGGGAC
GCTTGAAGACTGGGGTGCAATGAATTGTCGACTTCCCTCAGCTCTCAGCCCTTTTCCTCCCCCCGCGCGCCGTGCACGCGCAGCCTGCTCTGCC
CTTCTGAAAGAGACGCTGCAGCTGCGCTCTCAATGGGGCCTCAGCCTGCGGGGTCCGGGGAGGGGATGAAGTGGGTGGTTGCCCAGCCGGTGTA
CGAAGGTCGTGTGCCTGCCGAAGAGTGGGTCCCACACGCACAGCGCGCGCCGTACCAACCAAGCAAGGCTCACAGAGAGCATTTAGTCGTCCAG
AGCAAAGGATGCTGGGCACTCAAGAGAGCAGGGGGCCAAGTGGCTGTCAGTTCTGGTCAGTGACGGAACTGGACAGCATCTCCAAGGGCCAGGG
GATGGGGTAGGAGGACGGAGGAGCCAAGCTCCTGGGAAACGACCCCCCCAACCTATCCGCGACCGGTTGCAAATTCAAAGGAGGGCTGGGAGCC
CGCGAGGGTGCGGGAGGTGTCCTCGCTTGGGCTGCGTCTGCGCCCGCGTTTGTGTGTGTGTGCGCGCGCGTGTCTTCAGCTCCTTCCCCGGTCG
TAAAACCTAAACGGAAGACGGGTGTTAAAATGCTGCTGACAGGATCGCCTGAAACATGTTTACAGTAGGAAGGACTTTCCTCCAGAAAGCACAG
TCTGAGTCGATTCCAATTTATTTCCTCCCTGTAACTGTAAGCGAAAGGAGAGGTTCGGCTGCCGCGGCGGAGCGAGGCTGAGCACCATTAATCC
TTGGCGAGCTTAAGCCAGGGAGAGCGCCGTGCCTGCGAGACCGAGTCGCTGCGCTCCGGAAGGCGTTCTGCCTGAAGGAAACCTCTTTTTTTTT
TTTTTTTTTTTTTTTTTTTTTGAGTAGGGAGTGGCCTTTGCACTGGAGTCCTTAAGTAAGGCGCGTCGTTTCTCTGCAGCCGCTCTCGGAAGCC
GGAGAAACTGCATTCACTTTAGCAAGAAGACAGGGAAACTGAAATTGATAATCCCCCGAGTTGGGTTACCTGATCCAGACAGGAAGCTGGTCTA
GGCTTCCCCCGTCACCCGGAGCTCCTGACAAGTGAGCAGGAGGCGGCGGGTGGTTGCCCTAAACTTTGATTTATGAAAAAAAATCAGAATTCAT
CGACATACCCTGTTACTCGTTGAGCGTTTAAGCCTCTAACTAAAGAAGGTACAGTGGTAGGGAGTGGTAGGGGGGTCGGGAGAGGAGGAAGGGG
GAGAAGAAAGAAGACTGACTATCCCCACGATTTCCTACCTCGCCTCGCGGGCGCAGGGAGGCTCAGCGATGCCACCTGGCGGCTGCTTCTCCCC
AGATTGGGAGCCGGCTGGCTGCAGACAAACCCAGGCTCTCCCCGGGTGCCGTGCCTCTGCCTCTAGAATAGAAGCAGAAGCCTGTATTCATGCT
TGCAATCCAAGTGGCCCTGCAAACATTCGTCTTCTGCTTGTTTTCTTGAGAAAATCGAAGGAAAGGAATAATGAGTTGGCATTGGCATGAACAG
CTTAAGCCTACTGTGTGCTAGTCACTGGTTATGAGAGGAAAATGGTACAATTGTTAGAAATGCAGGTTTCTTAACTCTTAGCTTATTCTAGTGA
TAAAAATAAATGCTAGGTTTCATTTACCCCAGAATCCTATTTTGATATTTTGCAAATATGAGTTATTTCCATATATTATTTTTCAATGCACGCA
CCCCCACCCCCATTACGCGCTGAGCCCAGTTACAATGCAATGTCATCAGCCCAAAAGGATGAAGTGCAAGTTATGTTTGTCTACTTGACTGAGA
AGTAAATCAGCCTCACATGGAGGCTTCTTTTCCATAAAGTGTGGAACCAAAAAAACTGGCGCCTTTCAGTTGACAAAGGCCAATGACATCAGAT
TTCCCAGGGCTTCTTCCTTGTCAGGTAAAGTAACAACTACACAGTTTTTCCTCCTCTTTATTTTATTTGAACTGAATACTTGTCAGGTCTGACT
AGCGCCTACAATCATAGTTTCTATAACCAAGGCAAGGCAAAATCTTGCAAATCAGTGCAAATCCAGATTTGTTAAACTAATTTCCATTCAGGTC
CCTCCCTCCATGCCAAAGGAATATATTAAGCAGTGGAATAGAATCTTATTTCATCTATGCCCCATACCATTGTATGGATGTGAGATTTTATGTT
GTGTAGATGTGTGAGAGCAGAAGAAATGCATTAGCAGATATTACACTTACATCTCAAATGGAATTCCATCATTTAAGGGAATAAAATAAAATAC
CTAATTCAAAATTGGAAATGTCTAACATCATTGCTAACAAAGACAGTTTATTATTCTAAACCAGGATTTTTTTTTTCTGCAAAATTATCTTGGC
TTTCTCTTAATACAGCTAAAAGACTAGCATTAAGAATTACTTCATGTACTGCAAAGCCAGCAGAGGGAACACTGCGTACTGATTTAGTAGGGGC
CACCAAATATCTGGTATTTGGGGAAAAAAATGTCAGTAATGGAGAACTGATTACAGAGCATATGAAGTTAGGGAATGAGAAGGGAGAAATCTTT
TCTCATTATTTGGTATTGAGAGGTCCAACACATTCCAGTGTGCACACACTTAGTTGGTCACAAAGATGACTTTAATAAAAGGGCATTCGTCATA
TGTTTTCTTACATTCTAAGAGAACTAACCCATTAGAACAGATCTATTTCAGAAGTCTAGGCACTTGTGTAATATATAAATATATATGCAGTAAT
TATAAAGTCAATGGAAGTTACTGATACCATATTTCATCAAGTCTAAGAGGAAATTTTTCTTTTCATTTCAACTCTTCCTGAAATAAGGAGGCAT
CCTACATCTAGGGGGATATCACAGTTTCATTGGCAGCTTTCTTCCTTGGTGGAATGTAATATAATGGTGTGTCTTAAAATTTGTTGGATTTGTT
GAAATACAGAAAATACATGTAACAAAAGAATGAGGCCAGTGGCTGTGTTTTAGTACACCCTTTAAAAAATCCATGTGTTTTAAGCAGAAACATA
TATGACTGCTTTAACTTCCACTCTTCCCTATCATATTCGATGATGGAAGCATAAATGACTGAGAAATATAATCAATAATGTTGATACTCAACTT
GACTGAATTATCAAACTAGCTACACCCTCAAATCATTAGTTAGTACAAATTCAAAAATAAATATTCTTTTTTATTCTAGTGAAAATATTGATCT
TATAGATAATAGCCCTCTTAAAATAATTTTTTTTTGTCAGATTGGTAACATTTAAGGATGTGTTTCTATGCACACCAGAGAAAATGTAGTATAT
AACACAGCAACACTGTTATGCAAAAAAGGTGCCTGTGTGTTTTTGGTTTGGGTTTTTCTTTTTTTAAGGATTTGTTTCTCCTTTTTTAAAAAAA
TTATGTAATACAAGTAGCTAATCGTCAAAAATTATTTGCTTTTACCCACAAAAACAGTTTTGCTTGTGGTTGATCCTTACAGATCCAGTCAGAT
GGCTACATAGGGTGTTCTTTCCAGTAAAATTCTAAGTCTGACAACCTGGGCCTATTGCCAGCCTACATACTTTAAAAGTTTGACAACATACATA
GAAATTTACTACCCAGCACTAGTTTAATCTGAATCAAAAGCCGACTTAATTATTTTGGATTGACTTTACCTAACCACTTATGGTTAGCCTGAAG
AATATTAGTTTCCTATTATAGTGTTAAGGAAAGCTGATTTTTACTACAATTCTAATTTTCAGACACTGGAAAACAAGTAATTTCTGTAGCCAAC
GTTTATTTTGAATACCTTGGCATCCTTTCTTTGCCACATTCTTCTTAGTATTCTTCATGGAGTTCCTTTCTTTTAAAATCAATGGAATGGCAGG
GTTGCCACTATTGTCCTTCTATGGTTCACATCAATGATCTGTGATTACTTTTTTTTTTTTTTTGAGATAGTCACACGCTATCACCCACACTGGA
GTGCAGTGGCTCAATCTTAGCTCACTGCAACCTCCTGGGTTCAAGCAATTCTCCCGCCTTAGCTTCTCGAGTAGCTGGAATTACAGGCGCATGC
ATCCCTGCCCAGCTAATTTTTTAATTTTTTAATTTTTAGTAGAGACAGGGTTTCACCATGTTGACCAGGCTGGTCTCGAACTCCTGACCTGAAG
TGATCCACCCACCTTAGCCTCCCAAAGTGCTGGGATTACAGGCGTGAGCCACCGCGTCCTGCCTGTTATTACTTTTTTACATAAGTGGCTATTC
TGTTTTAGCCTCTTGAACAACATGCAGCTTTTACTAGGAGTGTGTCTGGCATTTGAATTAGAAAGGAAGATATTAGTATTCATTGAGGGCTTAC
AATACATCAGGTGCTTCATAGTCACAAATTCATTGAACCCTCACAACAACCCTCTATAAAGTAGCTCCTAGTGTTCCCATATAATAGATGAGGA
AAACTGAGACTTGGCCACATCCCCATAGTTACAAACCATTAAGAAGGATTAGCAGCCAGGTCTACTATGAATCCGGAGGCCCCATTCTCTTCTG
CTACCAGTTATCACCTCCTTCTACAGTTTCTCTTTGCCTTGGTTTTTCAGTTCCCTTCGATTCAAGTAACAAGAGGAAATATATAAGAAGGAAA
GGAGGAAGATGGCTTTGGGAGGAGGAGAGGGAAGAGAGTGAAGCAATCTTTAATTTTGAAAACAGGGAAAACAAATATTTAGAAAGTAAAACCG
ATTTCCATCCTTTACATGCCATTATACATCCTATACAAAACTCTGGAAATCTGCCTATTGAGGTAATTACGGAGCACTACCTAATGTGTTTCAA
GGGAAAGCTCCCCTGTTTGGGCTATGCAGGATTTACAGGGAGTTTAAATCATTAAATAACCATAGTTGACCCTTGCTTGGTTAAGAGAGTAAGC
AATTATTGCAAAGTCCTCTTCCCTGCCTTCCACAAACAGCACAAAAACGGTTCTGAAGGGATTTACACAATACATCAACATCTGTGCTGTACCT
AGAGTTGTTAAAATAATTGTGATGACAGGTGTCTAGGTACCACTTTCTTAATGGTTTTCAATATCCATTGATTTATTGTTTTGTGGCTTTCTCT
TCCCCTTTACAGCTTTGGCTTTAAGAAAGAGCTGGGGAACAGATTTTGCACCAGAGGCTGATTCTTTATTCCAATGCTGAAAAGAACTAATAAA
AATAATCACTTGGCTTTTAAGCAGAAAGCTTCCATTTACAGTATTTCACCCTTACCCCGCCCCGTTCCAAGCCCCTTGTTATCTAACATAATGG
TTAGAATACATTTCCGTCTCTCCCCTTAATAATTTCATGCATTAACATCCTCTTCACACACTATACACACACGCACGCGCGCACACACACGCGC
GCGCACACACACAGCAAAAGGAAAAAAGAGGCAGCAGAAATCTCAGCTGTACTTCTAGCCCTTTTAAAAAGTTTGCTCTGTAAATTGGAATGAG
GTCAGATTTGGAGCTTCTCATTGCACGCGGAGATTATTATTGCATCGGGTTCCAAGCCAATGGGAAGCCCGGGGGAGGGGTTTGGCATGAGGAA
GCGTTGGTTACAGCAGCTGATTGGCTGCAGCCAAGACTGTGAAAGGATAAAGAGGCGCGAGGCGGAATTGGGGTCTGCTCTAAGCTGCAGCAAG
AGAAACTGTGTGTGAGGGGAAGAGGCCTGTTTCGCTGTCGGGTCTCTAGTTCTTGCACGCTCTTTAAGAGTCTGCACTGGAGGAACTCCTGCCA
TTACCAGCTCCCTTCTTGCAGAAGGGAGGGGGAAACATACATTTATTCATGCCAGTCTGTTGCATGCAGGCTTTTTGGCTTCCTACCTTGCAAC
AAAATAATTGCACCAACTCCTTAGTGCCGATTCCGCCCACAGAGAGTCCTGGAGCCACAGTCTTTTTTGCTTTGCATTGTAGGAGAGGGACTAA
GTGCTAGAGACTATGTCGCTTTCCTGAGCTACCGAGAGCGCTCGTGAACTGGAATCAACTGCTTCAGGGAAAAAGAAAAAAAAAAAAAAAAGAC
TTGCCTGGGAGGCCGCGAGAAACTTGCATTGGAAGCTTCAGCAACCAGCATTCGAGAAACTCCTCTCTACTTTAGCACGGTCTCCAGACTCAGC
CGAGAGACAGCAAACTGCAGCGCGGTGAGAGAGCGAGAGAGAGGGAGAGAGAGACTCTCCAGCCTGGGAACTATAACTCCTCTGCGAGAGGCGG
AGAACTCCTTCCCCAAATCTTTTGGGGACTTTTCTCTCTTTACCCACCTCCGCCCCTGCGAGGAGTTGAGGGGCCAGTTCGGCCGCCGCGCGCG
TCTTCCCGTTCGGCGTGTGCTTGGCCCGGGGAACCGGGAGGGCCCGGCGATCGCGCGGCGGCCGCCGCGAGGGTGTGAGCGCGCGTGGGCGCCC
GCCGAGCCGAGGCCATGGTGCAGCAAACCAACAATGCCGAGAACACGGAAGCGCTGCTGGCCGGCGAGAGCTCGGACTCGGGCGCCGGCCTCGA
GCTGGGAATCGCCTCCTCCCCCACGCCCGGCTCCACCGCCTCCACGGGCGGCAAGGCCGACGACCCGAGCTGGTGCAAGACCCCGAGTGGGCAC
ATCAAGCGACCCATGAACGCCTTCATGGTGTGGTCGCAGATCGAGCGGCGCAAGATCATGGAGCAGTCGCCCGACATGCACAACGCCGAGATCT
CCAAGCGGCTGGGCAAACGCTGGAAGCTGCTCAAAGACAGCGACAAGATCCCTTTCATTCGAGAGGCGGAGCGGCTGCGCCTCAAGCACATGGC
TGACTACCCCGACTACAAGTACCGGCCCAGGAAGAAGGTGAAGTCCGGCAACGCCAACTCCAGCTCCTCGGCCGCCGCCTCCTCCAAGCCGGGG
GAGAAGGGAGACAAGGTCGGTGGCAGTGGCGGGGGCGGCCATGGGGGCGGCGGCGGCGGCGGGAGCAGCAACGCGGGGGGAGGAGGCGGCGGTG
CGAGTGGCGGCGGCGCCAACTCCAAACCGGCGCAGAAAAAGAGCTGCGGCTCCAAAGTGGCGGGCGGCGCGGGCGGTGGGGTTAGCAAACCGCA
CGCCAAGCTCATCCTGGCAGGCGGCGGCGGCGGCGGGAAAGCAGCGGCTGCCGCCGCCGCCTCCTTCGCCGCCGAACAGGCGGGGGCCGCCGCC
CTGCTGCCCCTGGGCGCCGCCGCCGACCACCACTCGCTGTACAAGGCGCGGACTCCCAGCGCCTCGGCCTCCGCCTCCTCGGCAGCCTCGGCCT
CCGCAGCGCTCGCGGCCCCGGGCAAGCACCTGGCGGAGAAGAAGGTGAAGCGCGTCTACCTGTTCGGCGGCCTGGGCACGTCGTCGTCGCCCGT
GGGCGGCGTGGGCGCGGGAGCCGACCCCAGCGACCCCCTGGGCCTGTACGAGGAGGAGGGCGCGGGCTGCTCGCCCGACGCGCCCAGCCTGAGC
GGCCGCAGCAGCGCCGCCTCGTCCCCCGCCGCCGGCCGCTCGCCCGCCGACCACCGCGGCTACGCCAGCCTGCGCGCCGCCTCGCCCGCCCCGT
CCAGCGCGCCCTCGCACGCGTCCTCCTCGGCCTCGTCCCACTCCTCCTCTTCCTCCTCCTCGGGCTCCTCGTCCTCCGACGACGAGTTCGAAGA
CGACCTGCTCGACCTGAACCCCAGCTCAAACTTTGAGAGCATGTCCCTGGGCAGCTTCAGTTCGTCGTCGGCGCTCGACCGGGACCTGGATTTT
AACTTCGAGCCCGGCTCCGGCTCGCACTTCGAGTTCCCGGACTACTGCACGCCCGAGGTGAGCGAGATGATCTCGGGAGACTGGCTCGAGTCCA
GCATCTCCAACCTGGTTTTCACCTACTGAAGGGCGCGCAGGCAGGGAGAAGGGCCGGGGGGGGTAGGAGAGGAGAAAAAAAAAGTGAAAAAAAG
AAACGAAAAGGACAGACGAAGAGTTTAAAGAGAAAAGGGAAAAAAGAAAGAAAAAGTAAGCAGGGCTGGCTTCGCCCGCGTTCTCGTCGTCGGA
TCAAGGAGCGCGGCGGCGTTTTGGACCCGCGCTCCCATCCCCCACCTTCCCGGGCCGGGGACCCACTCTGCCCAGCCGGAGGGACGCGGAGGAG
GAAGAGGGTAGACAGGGGCGACCTGTGATTGTTGTTATTGATGTTGTTGTTGATGGCAAAAAAAAAAAAGCGACTTCGAGTTTGCTCCCCTTTG
CTTGAAGAGACCCCCTCCCCCTTCCAACGAGCTTCCGGACTTGTCTGCACCCCCAGCAAGAAGGCGAGTTAGTTTTCTAGAGACTTGAAGGAGT
CTCCCCCTTCCTGCATCACCACCTTGGTTTTGTTTTATTTTGCTTCTTGGTCAAGAAAGGAGGGGAGAACCCAGCGCACCCCTCCCCCCCTTTT
TTTAAACGCGTGATGAAGACAGAAGGCTCCGGGGTGACGAATTTGGCCGATGGCAGATGTTTTGGGGGAACGCCGGGACTGAGAGACTCCACGC
AGGCGAATTCCCGTTTGGGGCTTTTTTTTCCTCCCTCTTTTCCCCTTGCCCCCTCTGCAGCCGGAGGAGGAGATGTTGAGGGGAGGAGGCCAGC
CAGTGTGACCGGCGCTAGGAAATGACCCGAGAACCCCGTTGGAAGCGCAGCAGCGGGAGCTAGGGGCGGGGGCGGAGGAGGACACGAACTGGAA
GGGGGTTCACGGTCAAACTGAAATGGATTTGCACGTTGGGGAGCTGGCGGCGGCGGCTGCTGGGCCTCCGCCTTCTTTTCTACGTGAAATCAGT
GAGGTGAGACTTCCCAGACCCCGGAGGCGTGGAGGAGAGGAGACTGTTTGATGTGGTACAGGGGCAGTCAGTGGAGGGCGAGTGGTTTCGGAAA
AAAAAAAAGAAAAAAAGAAAAAAAAAGAAAAAAAAAAGATTTTTTTCTTCTCTTAATCGGAATCGTGATGGTGTTGGATTATTTCAATGGTGGG
GTTAATATAGCATGTTATCCTGTCTATCTTTTAAAGATTTCTGTATAAGACTGTTGAGCAGTTTTTAAAATAGTGTAGGATAATATAAAAAGCA
GATAGATGGCGCTATGTTTGATTCCTACAACGAAATTATCACCAGCTTTTTTTCATTCTTAACTCTTTAAAGGATTCAAACGCAACTCAAATCT
GTGCTGGACTTTAAAAAAACAATTCAGGACCAAATTTTTTCTCAGTGTGTGTGTTTATTCCTTATAGGTGTAAATGAGAAGACGTGTTTTTTTC
CTTCACCGATGCTCCATCCTCGTATTTCTTTTTCCTTGTAAATGTAATCAGATGCCATTTTATATGTGGACGTATTTATACTGGCCAAACATAT
TTTTTCTTTTGTCCCTTTTTTTCTTTCCTTTCTTTTTACTTCCTTTATTTCTTTATTCCTTCCTTTTCCTTTTTTTCTTTTTTTTTTCTTTTTT
TTTTTTTTTTTTTGGTAGTTGTTGTTACCCACGCCATTTTACGTCTCCTTCACTGAAGGGCTAGAGTTTTAACTTTTAATTTTTTATATTTAAA
TGTAGACTTTTGACACTTTTAAAAAACAAAAAAAGACAAGAGAGATGAAAACGTTTGATTATTTTCTCAGTGTATTTTTGTAAAAAATATATAA
AGGGGGTGTTAATCGGTGTAAATCGCTGTTTGGATTTCCTGATTTTATAACAGGGCGGCTGGTTAATATCTCACACAGTTTAAAAAATCAGCCC
CTAATTTCTCCATGTTTACACTTCAATCTGCAGGCTTCTTAAAGTGACAGTATCCCTTAACCTGCCACCAGTGTCCACCCTCCGGCCCCCGTCT
TGTAAAAAGGGGAGGAGAATTAGCCAAACACTGTAAGCTTTTAAGAAAAACAAAGTTTTAAACGAAATACTGCTCTGTCCAGAGGCTTTAAAAC
TGGTGCAATTACAGCAAAAAGGGATTCTGTAGCTTTAACTTGTAAACCACATCTTTTTTGCACTTTTTTTATAAGCAAAAACGTGCCGTTTAAA
CCACTGGATCTATCTAAATGCCGATTTGAGTTCGCGACACTATGTACTGCGTTTTTCATTCTTGTATTTGACTATTTAATCCTTTCTACTTGTC
GCTAAATATAATTGTTTTAGTCTTATGGCATGATGATAGCATATGTGTTCAGGTTTATAGCTGTTGTGTTTAAAAATTGAAAAAAGTGGAAAAC
ATCTTTGTACATTTAAGTCTGTATTATAATAAGCAAAAAGATTGTGTGTATGTATGTTTAATATAACATGACAGGCACTAGGACGTCTGCCTTT
TTAAGGCAGTTCCGTTAAGGGTTTTTGTTTTTAAACTTTTTTTTGCCATCCATCCTGTGCAATATGCCGTGTAGAATATTTGTCTTAAAATTCA
AGGCCACAAAAACAATGTTTGGGGGAAAAAAAAGAAAAAATCATGCCAGCTAATCATGTCAAGTTCACTGCCTGTCAGATTGTTGATATATACC
TTCTGTAAATAACTTTTTTTGAGAAGGAAATAAAATCAGCTGGAACTGAACCCTAAATCTTGACTTTTGTCGTTATTATGCCCAATGCCTAAGA
TTGGAAAGGCCCTACAGTATCTGGACACTACACAATCTGCCTTAGCTTTAAAAAAATAAGTTCTCAGAGGAGTCTTGTCAAATGCTACCTTATC
GAGGAACATTAAATGTCTCTTGGCCAGATTTCCTCTGACCCTGAAAATGATGGCATCAGCTGAATATGTGTCAACTCAACACATGCTTTCTAGG
AATTTTCTGAGTTTACCATATTTAAAAACGCCCTTTGTCTTGGAATCTGAAAGAGGGGGAGGGGCAACGTGGACAGAATTCTTCACAGATGAAA
CTGGCTAGATGATCTTTTTTTCAAGTTGGTGCTCCTGGAGCTGCTTTCTCCTTGATTTGCCCATGCTTACCTCCAGCAGTGGCTGTAATTGGTT
TTCGTGGGTTAAGGACAAATAAACTGTTATTATCAGTAGATTATACAGGAGGAGGCTTTTACCGATTATGAAATGCAAAACGTATGCATTTATG
CATTTTTTTTCTCAAGCAGAAAGGTTGATGTGCTACAATCTTCATTTTTTGCCATGCCCAACATGGTGTTAATGTAAGAGAACTGCAGCAATAA
GGATGACCTGGAGAAGCTGGCTGCATGTGGGCTCAGAGGAAAGCCCCCTCTCCAAATAGCACTTACTTTGGCTGCTGGAGATTTGTGCTTTTTC
TAAGAATCAGGAAGGATTGTTGAGACTGCAAGGTGAAAAAGTACTTGGATTTCTTAAGGCCCATTACTCTTGGGTTTGGAACATACAACTTGCT
AAGTGGCACTATTTCCTGTTTATTTTGAGAGTTAACAATTGCTATTAACTAACAAAAGCCATCTTCCAAATGTTAATTAAAAATGTGTCTTTTA
CTTGTCTTGAGTTTTTCAGCACTGGAGAGGAGGGGTTAACAAGCAGAAGTGTTTGGAAATTATCCCTTAGTCATTTGGATTGCTTGTAGATAAG
GGAGTAGTATCCTGGAAGGTACATCTGGAAAAAGAGACCTTTTGAAGACTAGTCTAACAGGAGGGGGTTTTGTGGTTGTCCAACGAAAAGCAAT
AATGCACCCCCAAAAACCAGTATAACTTGTGTTTCAATATAATTTATCTCTTAAAGATAGCTACTAGTTTTCAACATTTTTTTCATGTGTGGTG
CTCATATATTTAGTTTTAATTGCATTTGCGTTCTGCAATTGGAATTCCATTTTTTAAAATGACCTTAGAACAACATGGCATCTAAGTTTCTTCA
AAGATCTTTGTAAAAGGAAGAACTCTTCTTTAGAAAAGTGCTTTTAATTTTTGTTCATTGCAGTGATAATATAATGGTTTAGCTTTTTTCAGAT
CCAAGGTATCAATACCATGACATAAATGAAGCCTTAAATGATGCCTTTTTTAAATGATGCATCTCTTTTAAAGCATATTTTTATTTCGGAGGGT
TCTCCTGTTAGAACTGTTTCTTTTCCCTTGACTCTCTATGTAAAAGCAGATGTTTTAAATATTTCCTTTAAGAATTTTGATAACATTGGTTTAA
AAATAAAAGCTGAAATGTGTGTGTCCTTCCTGTCAGGTGGTAAAGTAAGAAGCATTTTTGTACAATGTCTTTTTCTCTGAGTCTGTTTTCATTC
GTAAGGGATACTTACTTGTTAGGTGGTACAAGGCATCCTAAACCTGCGAAACCAAACAGTTTAGTGTGATTTTTTTCTTACAGATAATTTGGAT
CAATAGAAAAGAGTAAGTTTCAGATTTTCTTTTAAGAATATCCAGCAATTGCTTCTGGCCCTGTGTTCCATCACAAATGTGAATCAGTGATACT
TGCTTAAGCAAGTTTTGTCAATTCCTAGTTGAATTCCTTTAAGTGCCAAGAAAAAAAATAAAAAGCATGTAAGCTGAGAGGGTGGGCCCAGCTC
CTGGGAAGGCTTTTAGGGTTTGTGTTTACCCTGCTTTTGAGGGTACTAGGGGAGCTAAGGGATTCTGAGGAAAGCAAGAAAGCAAAGGGTTAAA
TGGACATCAACATTTACCAAGGGTTTCCTGTGTTTGTAGGGTACACACAAATTAGAAGACATGGTCTGGGGAATACTATGGGTCAGAATGCTTA
ATCATGAATTTAAAACAAATTCAGGGATCCCAAGGAAGTATTTGAAAAGAGAGAAATGTTTAAAAAAAAATTTTTTTTTTTTAAGAATACAAAC
CCATCTGGCCGGGCGCGGTGGCTCACGCCTGTAATCCCAGCACTTTGGGAGGCCAAGGCGGGCGGATCACTTGAGGTCAGGAGTTTGAAACCAG
CCTGGCCAATATAGCGAAACCTCCTCTCTACTAAAACTACAAAAATTAGTTGAGCGTAGTGGTGCACGCCTGTAATCCCAGCTACTCGGGAGGC
TTAGGTAGGAGAATCTCTTGAAGCCGGGAGGCTGAGGTTGCAGTGAGCCAAAATCGCATCAGTGCACTCTAGCCTGGGTGACAGAGTGAGATTC
TGTCTCAAAAACAAACAAAAAAATACAAACCCATCTACCCATTTGGATTTCTCAGAGTGTAAGGTTGGTATGCTTTATTTCTCTTTCTGGTGGT
TTTTGTTTTGTGCCTCCTCCTGGCACCCCTCCTATTAAGGCCTACAAAATACCTCTCTAACCCATATTCACTAGGGTTTACTCCTACCCCTCTG
ATTCTTCCTGAAAAATGATATCTCCATACGCATTTCTCAATTTTCACTATCATTTGAAAGCTAAAGTGGAGGGGTGGGAAGAAAGCATCTCCAG
ACATCCGCATGGTGTTTCAGGCTCTGCCAAGTGTCTACCACCATTCCCCTGTGCCCAGGATAAAGTGCAGAATTCACATGCTGCTCTTCAAGGC
CCTGCACAAAAGGGTTTTAAGCCCAACTTTCCAGTTCTTCCAAATTGCAGTATAACCAAACTGCTCTTTCCTGATAGACTGGAATTCTTGAAGG
ACAGAAACTAGGCATTGAATACTTCTTGCATTCCCCAGTTTGCTTTCCCTGCTTCTTTTCACATAATAAATGCTAAATTCTTATTTCTTGACTT
TACATGGTTTCCATGGGTTCCTAAGAGCTTCCAACAGGCATTTGCATTTTATGCTGCTTGTGGTAAGCGCCAGCGCCGGATTGCATGAAAACCT
GGATTAGAATGCTGCCTCTGAGACCAACAAGCTGGAGGACCTTGGACAGATTTCTCAGCCTTTTTAAGTTTCCGTGTTTTAAATCGTCATATAG
AAAGAGAGCTATCTAATGTAGACAGCTGTGTTGGGAATAGAGTGCAGCGGCACGTGTGTGACATGTCGTAATGCCTGGCCTACCCCATGGGCTC
GGTGAATGCTAGAACCTGACCTCATGGGTGGTTCTAAAAAGCTAATGTGTGTAAAGCCTAACACAGTGCCAGCAACACAGAAGATGCTTTAAAT
TCTAAAAATGGGGAGCAAAGCAGCGTGGCCGGGGTCCGAGCTGGGGTTCTGTCCCCACTGGGCTGTCGCCCCAGCTGGACTACCACCATGGAAC
TCAGGGCCGAATACCTCAGGGAGAAGCTGCAGCGGGACCTGGCGGCGGAGCATGTGGCGGTGGAGGACAACACGACCCTCAACCATTGCGCCTG
TAGCTTCTGGCGGTGTCGGCCAAGTTCGAGGGGAAGCCGCTGCTTCAGAGACACCGCCTGGTGAACGCGTGCCTAGCAGAAGCGCTCCTGCGCA
TCCATGCCTTTGAACAGAAAACCCTAATCCCAGAGCAGTGGGCCCGTGAGCGGCAGAAATGAGGGACTGGGACCTGCACAGCCATTAAATTACA
AATCTGGAAAAAAAAATTTCTAAAAATAAATCTCCTGAATCAATGTTTCTCAGTCTGCTACACGAATCCCCAGCATCAGAATTACCTGGAGATT
CTTGTTAAGGGTAGCGATTATACAGATCGCCAGCCACCGTCAGACTGTGGGGATGGGGCCTTAATGAGCCCCCCATAGATTTCCAGTGCTCACT
GAAGTTTGGAAATCAGTACCCTCACTGTATTTTAGCATATTCTCACAATAAGTATGCAGGCGAGGGATTATGACGCCCTCATTGTTTTAACATG
GAGAAATTGAGTTGAGAATGGCCCAATGGCTGATCTGAGGTCACAAGATCAGAAGTGCCAGAAAGGGTATTGGAACTCTATCTCCTGAGCCCTA
GAATCAGCCTAGAACTTATTTCTCTAAATAGATGTCTTCTCCATTTCTACCCAAGGCTTGGTCCCTTGGCAAAAATTGGCAGTCCCTGCACTTC
CCTCAGCCCTCTGACTCCTATCCATTTTCTCAAGGATGCCCTTCCACCATCCTTGTTAAAAGTTTAAGTTTGGGCCGGGCGCCCGTCGCTGGTG
CCTGTAATCCCAGCCCCTAGGGAGGCGAGGCAGGGGGATCACCTGAGGTCAGGAGTTTGAGACCAGCCTGACCAACATGGTGAAATCCCGTCTC
CATTAAAAATACAAAAAAATTAGCTGAGTGTAGTGGCACATGCCTTTAATCCCAGCTACCCAGGAGGCTAAGGCAGGAGAATCGCTTGAACCTG
GGAGACAGAAGTTGCAGTGAGCCAAGACTGTGCTATTGCACTCCAGCCTGGGCAACAAGAGTGAAAATCCATCTCAAAAAAAAAAAAAAAAGTG
TAAGTTTAAGTAATTAGCCAAGTTAATGTGTGTGTGTGTGTGTGTGTGTCTGTGTGTATTCCAGTTAAACAAATGTGTGCTAAAAGATGAACCC
AGGGACTGATTTTTGGTAGAAGTATCCAAGGCAGAGAACAGAAATAATATAAATATAGTGCCTGGAACTTCTGCTTTGTTGTTTAAAAGGCAGA
GCTTCCAGCAAGTGAGAAAATTGGAAATTATTCAGAGCACACTCAGCCTTTATAGAGAGGGCTATCGTCTACCCATTATATCCTAATGAGATTG
ACCGTTGGCTACAGTTAACTTGAAGGGTTTTATGGTTTCCTGCATGTGTTGTTTTCCTTCTCTATGTTAAAAAGAAGAAAAAAAAAAACTCTCA
CATCACTAAACGGTATTTCCCAGGGGGATGTCTGACTAGGCTGCTGCATTTTGGTTTGGGCCTTAGTTAACAATGGACCTCTATGTGTGTATGT
TTGTGGATACAGGTGATAATAAATTTAGCCCATTTTATTTTCTTTTGACAGTTTCAATTCAGGGCAGTTGGTTTATGGGGTACCGCCAAACAAG
CCAGGCCTAAGTCAAGTCATGGCAGGAACTTTCTTGTAGGGCCTTCCTTTACCCTGCAGATTTTTTGTTGAAGGACAAAGTTCTAAGTCATTTG
TGGACCAATATACACAGCTGCTTTGCATCATACGTAAGACATTCTAAGGGTATTCCATTTGAGTGTTTTTTTTAAAGGGCAGGACATTAAGAAA
CAAATGAAATATCATTTACTTAGCCCACTTACTTCTTTAGATATTTTTCCAAATACATAATTCTTTGGATTAGTTTTTAAACACATAATGAATG
TGCTATTTCCTGACATAACATGCAGTTGAAAGGCAAGTGAAAACACTATTATCCTTAGTAGGTAGGCTCTCTCCAGGCAGTGAATGCCCAGCAG
TGTAGGGCGATGGTTGGCTAATACATGTGATGCATGTAACTGCTTTTATAAGAGCTAAGCAGAAGGAGCCTGTTGAATGATCAGTTAGCTCCCG
TTGTATATGTGGTTAATTGTTACTTTCTTTCCTACCGGCCCCTGCCCTAACTTCCTCAGATCAACACCTAAAGCCTGTGAAAATAGTCTCTTCT
GGAACCCCTGACCTTCAGTCTCTGCCCCTGCTTTCTCTCTTCCCATTCCCACACTTTCTATGTGGACCTTTTATCTCTCCTGTTTGCCAATCCC
CCTCAGACTTTGAATAATTTATCTGAAGTTCCTGGGAAGTTGTGGGGCTGTCTGTAGTCTCAAAATACACTGCACTTTGTTCCTTCTCTAGGCC
GAGGTAGGTCCAAAAGCCTGTCTTTGAAAGAGGTGGTACTAAGTACATTGAGGGATTTACTAACAGTTTTAATTTTTTGGATGAGCACCCAATG
AATCCAAAAGCTGGAGGAAAGAAAAAATAAGGGGCAAAAGATGAAGGTGAATACTACTTTGTAATTCTGATATCTCCCAAAGAATGTCAAGTAA
ATTAAGGGAACACTGATTAAGATTTCCAGTTCCTATTGTTATAACAATGTATTTTATACATGTCTTTTCAAATACATGAGAGTTTTCCTTGAGT
AGTTACTAGAAGTGGAAAGTGCTGGATCTTAGTCTTATAGACATAGGCTTTTGACAGTTTCCGTACCCTGGAGCTGGTTAGGCGTAAAGTGGTT
TGAGGAGAGAAAAACTAGTGATATGTCTAAATGAACACAGGTTAAAAAGTAAACCAAACTTTGAAACAATCATTTTAACCCCCGGAGAAGGTGA
ATAGATTATTTTAATATGTAAGCAGATACATCTGCAAATCATACTGAGATGATTTTTCTATCCCAAGTGTCTGATAACATATATGAATAGAAAA
GCGAACAATTACAGAAACAGCCTTGACACTGACATGGTGACAAAGTATGGGGATTTTAGTACTTCCGAAATAAACATCAGAACAGAGATCAACT
CTCTTCGGTCTTTTTCTTCTTCCATTAGAACTCATCAGGCTGATTCCCATTTCTGCATTACTAGCCTTACTTTTTTTTTTGCTAGGCTCTGTGA
TGCAAGGAACAGAATCACTCAAGTAACCTGAAATCATGGGGCTTTATTACAAAGAGACACGGGGCTTTATTACAAAGAAACACAAGACTACAAG
GGAACTGAAAATGTCATGGTAGTTCTGGGAGAAATAGTGTTCAAGATCCACGGCAGCTCTGGAGAGATTAAGTGGTTACCAATTTAGCATTAAG
ATTATGCAGATAATTAATATTGCTCAGGTATTCTCTTAGTATTCCTGTGTGTGTTGATTTTCTCCCAATACCAAATCTTGCTTCTGTTCTCCAT
ATTTTGTCCTTCATATTCCCTTTTAAGACAGCTCTGGGCCGGGCACGGTGGCTCAGGCCTGTAATCCCAGCACTTTGGGAGGCCTTGGTGGGCG
GATCACTTGAGGTCAGGAGTTCAAGATCAGCCTGGTCAACATGGTGAAACCCCGTCTCTATTAAAAATACAAAAATTGTTGGGAGGCCGAGGCG
GGTGGATCACGAGGTAAGGAGATTGACACCATCCTGGCTAACACGGTGAAACCCCGTCTCTACTAAAAATACAAAAAAATTAGCCGGGCGTGGT
GGCGGCCGCCTTTAGTCCCAGCTACTCAGGAGGCTGAGGCAGGACAATGGTGGTGAACCCAGGAGGCAGAGCTTGCAGTGAGCTGAGATCGCGC
CACTGCACTCCAGGCTGGGCGACAGAGCAAGACTCCGTCTCAAAAAAAAAAAAAAAAATTGGCCGGGCACTGTGGCTCATGCCTATAATCTCAG
CACTTTGGGAGGTCAAGGTGGGCATATCACGAGATCAAGAGATCGAGACCATCCTGGCCAACATGGTGAAACCCCATCTCTACTAAAAATACAA
AAATTAGCTGGACGTGCTGGCACAAGCCTGTAGTCCCAGCTACTCGGGAGACTGAGGCAGAAGAATCACTTGAACCCGGGAGACGGAGGTTGCA
GTGAGCCAAGATGGTGCCACTGCACTCTAGCCTGGCAACAGAGTGAGACTTCGTCTCAAAAAAAAAAAAAAAAAATTTGCTGGGCATGGTGGCA
TGCACTTGTAATCCCAGCTACTCTGGAGGCTGAGGTAGGAGAATCGCTTGAACCCGGGAGGTGAAGTTGCAGTGAGTCGAGACTGTAACCACTG
CACTCCAGCCTGGGTGACAGAGCAAGACTACATCTTAAAAAAAAAAAAGACAGCTCTGCATGGCAGTTATTTCATCAATTACTTACCAAGCAAT
GAAACGGCTGCTTTGGGGTTGTGTGCCTACTTTTGGACAAATCACTTATTAATGTATTATATTCTTCACTCTCCCATCCCTCTTTGTTTTGCTT
TTGTTCCTAGGGTCTCATGGCCCTCATACGTCCTCTTATATTTTTTCTTTGATCTTCATATACTAAGGGAGGGTGAACCCCAGGCTTCATGGAA
CATCCACCCTTCCTCCTCTACCATCCACAATATCCTCATTTCAAACAGACTGGCTTGGCTTTTATCTATTTAAAATTCTAACCTTTTCCCTATA
GCTCGAATTTTGGCACTCAGGCCTAGAGATATAAAGAGTAGAGATTAAATATGCCACATTCTAAAGGGAAGCCAGGTGATGCCCTTACCCCAGT
AATCCAGAGCAGAAAGGATCCTACCATAGTCAGTATTCCCCTCCTCAACACGG
HUMAN SEQUENCE-mRNA
TTCCCCAGCATTCGAGAAACTCCTCTCTACTTTAGCACGGTCTCCAGACTCAGCCGAGAGACAGCAAACTGCAGCGCGGTGAGAGAGCGAGAGA
GAGGGAGAGAGAGACTCTCCAGCCTGGGAACTATAACTCCTCTGCGAGAGGCGGAGAACTCCTTCCCCAAATCTTTTGGGGACTTTTCTCTCTT
TACCCACCTCCGCCCCTGCGAGGAGTTGAGGGGCCAGTTCGGCCGCCGCGCGCGTCTTCCCGTTCGGCGTGTGCTTGGCCCGGGGAACCGGGAG
GGCCCGGCGATCGCGCGGCGGCCGCCGCGAGGGTGTGAGCGCGCGTGGGCGCCCGCCGAGCCGAGGCCATGGTGCAGCAAACCAACAATGCCGA
GAACACGGAAGCGCTGCTGGCCGGCGAGAGCTCGGACTCGGGCGCCGGCCTCGAGCTGGGAATCGCCTCCTCCCCCACGCCCGGCTCCACCGCC
TCCACGGGCGGCAAGGCCGACGACCCGAGCTGGTGCAAGACCCCGAGTGGGCACATCAAGCGACCCATGAACGCCTTCATGGTGTGGTCGCAGA
TCGAGCGGCGCAAGATCATGGAGCAGTCGCCCGACATGCACAACGCCGAGATCTCCAAGCGGCTGGGCAAACGCTGGAAGCTGCTCAAAGACAG
CGACAAGATCCCTTTCATTCGAGAGGCGGAGCGGCTGCGCCTCAAGCACATGGCTGACTACCCCGACTACAAGTACCGGCCCAGGAAGAAGGTG
AAGTCCGGCAACGCCAACTCCAGCTCCTCGGCCGCCGCCTCCTCCAAGCCGGGGGAGAAGGGAGACAAGGTCGGTGGCAGTGGCGGGGGCGGCC
ATGGGGGCGGCGGCGGCGGCGGGAGCAGCAACGCGGGGGGAGGAGGCGGCGGTGCGAGTGGCGGCGGCGCCAACTCCAAACCGGCGCAGAAAAA
GAGCTGCGGCTCCAAAGTGGCGGGCGGCGCGGGCGGTGGGGTTAGCAAACCGCACGCCAAGCTCATCCTGGCAGGCGGCGGCGGCGGCGGGAAA
GCAGCGGCTGCCGCCGCCGCCTCCTTCGCCGCCGAACAGGCGGGGGCCGCCGCCCTGCTGCCCCTGGGCGCCGCCGCCGACCACCACTCGCTGT
ACAAGGCGCGGACTCCCAGCGCCTCGGCCTCCGCCTCCTCGGCAGCCTCGGCCTCCGCAGCGCTCGCGGCCCCGGGCAAGCACCTGGCGGAGAA
GAAGGTGAAGCGCGTCTACCTGTTCGGCGGCCTGGGCACGTCGTCGTCGCCCGTGGGCGGCGTGGGCGCGGGAGCCGACCCCAGCGACCCCCTG
GGCCTGTACGAGGAGGAGGGCGCGGGCTGCTCGCCCGACGCGCCCAGCCTGAGCGGCCGCAGCAGCGCCGCCTCGTCCCCCGCCGCCGGCCGCT
CGCCCGCCGACCACCGCGGCTACGCCAGCCTGCGCGCCGCCTCGCCCGCCCCGTCCAGCGCGCCCTCGCACGCGTCCTCCTCGGCCTCGTCCCA
CTCCTCCTCTTCCTCCTCCTCGGGCTCCTCGTCCTCCGACGACGAGTTCGAAGACGACCTGCTCGACCTGAACCCCAGCTCAAACTTTGAGAGC
ATGTCCCTGGGCAGCTTCAGTTCGTCGTCGGCGCTCGACCGGGACCTGGATTTTAACTTCGAGCCCGGCTCCGGCTCGCACTTCGAGTTCCCGG
ACTACTGCACGCCCGAGGTGAGCGAGATGATCTCGGGAGACTGGCTCGAGTCCAGCATCTCCAACCTGGTTTTCACCTACTGAAGGGCGCGCAG
GCAGGGAGAAGGGCCGGGGGGGGTAGGAGAGGAGAAAAAAAAAGTGAAAAAAAGAAACGAAAAGGACAGACGAAGAGTTTAAAGAGAAAAGGGA
AAAAAGAAAGAAAAAGTAAGCAGGGCTCGTTCGCCCGCGTTCTCGTCGTCGGATCAAGGAGCGCGGCGGCGTTTTGGACCCGCGCTCCCATCCC
CCACCTTCCCGGGCCGGGGACCCACTCTGCCCAGCCGGAGGGACGCGGAGGAGGAAGAGGGTAGACAGGGGCGACCTGTGATTGTTGTTATTGA
TGTTGTTGTTGATGGCAAAAAAAAAAAGCGACTTCGAGTTTGCTCCCCTTTGCTTGAAGAGACCCCCTCCCCCTTCCAACGAGCTTCCGGACTT
GTCTGCACCCCCAGCAAGAAGGCGAGTTAGTTTTCTAGAGACTTGAAGGAGTCTCCCCCTTCCTGCATCACCACCTTGGTTTTGTTTTATTTTG
CTTCTTGGTCAAGAAAGGAGGGGAGAACCCAGCGCACCCCTCCCCCCCTTTTTTTAAACGCGTGATGAAGACAGAAGGCTCCGGGGTGACGAAT
TTGGCCGATGGCAGATGTTTTGGGGGAACGCCGGGACTGAGAGACTCCACGCAGGCGAATTCCCGTTTGGGGCCTTTTTTTCCTCCCTCTTTTC
CCCTTGCCCCCTCTGCAGCCGGAGGAGGAGATGTTGAGGGGAGGAGGCCAGCCAGTGTGACCGGCGCTAGGAAATGACCCGAGAACCCCGTTGG
AAGCGCAGCAGCGGGAGCTAGGGGCGGGGGCGGAGGAGGACACGAACTGGAAGGGGGTTCACGGTCAAACTGAAATGGATTTGCACGTTGGGGA
GCTGGCGGCGGCGGCTGCTGGGCCTCCGCCTTCTTTTCTACGTGAAATCAGTGAGGTGAGACTTCCCAGACCCCGGAGGCGTGGAGGAGAGGAG
ACTGTTTGATGTGGTACAGGGGCAGTCAGTGGAGGGCGAGTGGTTTCGGAAAAAAAAAAAGAAAAAAAGGG
HUMAN SEQUENCE-CODING
ATGGTGCAGCAAACCAACAATGCCGAGAACACGGAAGCGCTGCTGGCCGGCGAGAGCTCGGACTCGGGCGCCGGCCTCGAGCTGGGAATCGCCT
CCTCCCCCACGCCCGGCTCCACCGCCTCCACGGGCGGCAAGGCCGACGACCCGAGCTGGTGCAAGACCCCGAGTGGGCACATCAAGCGACCCAT
GAACGCCTTCATGGTGTGGTCGCAGATCGAGCGGCGCAAGATCATGGAGCAGTCGCCCGACATGCACAACGCCGAGATCTCCAAGCGGCTGGGC
AAACGCTGGAAGCTGCTCAAAGACAGCGACAAGATCCCTTTCATTCGAGAGGCGGAGCGGCTGCGCCTCAAGCACATGGCTGACTACCCCGACT
ACAAGTACCGGCCCAGGAAGAAGGTGAAGTCCGGCAACGCCAACTCCAGCTCCTCGGCCGCCGCCTCCTCCAAGCCGGGGGAGAAGGGAGACAA
GGTCGGTGGCAGTGGCGGGGGCGGCCATGGGGGCGGCGGCGGCGGCGGGAGCAGCAACGCGGGGGGAGGAGGCGGCGGTGCGAGTGGCGGCGGC
GCCAACTCCAAACCGGCGCAGAAAAAGAGCTGCGGCTCCAAAGTGGCGGGCGGCGCGGGCGGTGGGGTTAGCAAACCGCACGCCAAGCTCATCC
TGGCAGGCGGCGGCGGCGGCGGGAAAGCAGCGGCTGCCGCCGCCGCCTCCTTCGCCGCCGAACAGGCGGGGGCCGCCGCCCTGCTGCCCCTGGG
CGCCGCCGCCGACCACCACTCGCTGTACAAGGCGCGGACTCCCAGCGCCTCGGCCTCCGCCTCCTCGGCAGCCTCGGCCTCCGCAGCGCTCGCG
GCCCCGGGCAAGCACCTGGCGGAGAAGAAGGTGAAGCGCGTCTACCTGTTCGGCGGCCTGGGCACGTCGTCGTCGCCCGTGGGCGGCGTGGGCG
CGGGAGCCGACCCCAGCGACCCCCTGGGCCTGTACGAGGAGGAGGGCGCGGGCTGCTCGCCCGACGCGCCCAGCCTGAGCGGCCGCAGCAGCGC
CGCCTCGTCCCCCGCCGCCGGCCGCTCGCCCGCCGACCACCGCGGCTACGCCAGCCTGCGCGCCGCCTCGCCCGCCCCGTCCAGCGCGCCCTCG
CACGCGTCCTCCTCGGCCTCGTCCCACTCCTCCTCTTCCTCCTCCTCGGGCTCCTCGTCCTCCGACGACGAGTTCGAAGACGACCTGCTCGACC
TGAACCCCAGCTCAAACTTTGAGAGCATGTCCCTGGGCAGCTTCAGTTCGTCGTCGGCGCTCGACCGGGACCTGGATTTTAACTTCGAGCCCGG
CTCCGGCTCGCACTTCGAGTTCCCGGACTACTGCACGCCCGAGGTGAGCGAGATGATCTCGGGAGACTGGCTCGAGTCCAGCATCTCCAACCTG
GTTTTCACCTACTGA

TABLE 8

MOUSE NOMENCLATURE
ICSGNM Tcof1
Celera mCG6035
HUMAN NOMENCLATURE
HGNCT COF1
Celera hCG38609
MOUSE SEQUENCE-GENOMIC
TTAAGAAAGCTGGGGCCACTGGACAGTGGTGGCCCACGCCTTTAATCCCAGCAATTGGGAGGCAGAGGCAGGCAGATTTCTGAGTTTGAGGCCA
GCCTGGTCTACAGAGTGAGTTCCAGGGACAGCTGAGACTACACCGAGAAACTCTGTCTTGAAAAAAAAAAACTGAAGGAGAGGAAGAAGAAGAG
GAGGAGGAGGAGAATTNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNAATTACCCTTGGCTGCACAGCATGTGTGGGCGACA
CGAGACCCTGTCTCTAAAAAGAAACAGCAGCTGGGACCTGAACCTGTAGTCAGTAGCTTAGCTAGGGCGGGGTGGGGGACCGGGCTGATAATCA
GGGATGCGCTGTGAGGCTATTTGTGTGGAAGAGGGAAGGGAAGGAAGAACAGCAGCAGAGAACAACCAAAAGCATCAGCCTAGGAGAAAGGGAC
CAACTGGTCTACCTTTACTCCCTACCTGTCAATGACCTTACTCAGTCACTTCCTGTCACTAGGCCTGGTTCTTTAACCATGAAAGGATGGAGCT
GGCCAGGAGCTCTCTATGAACCCTTTCTGATGATCTTCAAAAGAAATGCACCCCCCCACACACACCCAAATAAATAAATAAATAAATAAATAAA
TAATGCTCAGGAGACCAGTGGGCAATTAAATTGGTTCTACCTATTATAGTGTCTCTCCCTGTTGCCCACTGATTCCACCCTACAGTTTTAAGAC
TTCAGTCCATAGCCCACTGTGGTTTTAGCTTAATCATAAGGCCTATGTCCTGCCTCCCTCATCTGTGCCTGTGGTCCAGCCTGGGTCATGTTAG
GAGGCCCTGAAAGCTGGCATCTGCTCCAGTCCTGCCTTGTACCACCGGTGGTAACTCAGTCCTCACCTCTCAAAGTCTCTGTTTCCTCATCTAC
TAGCAGAGCTGGTAACCAGTTCAGATATGTTCTACGGTGCTAAACTGATGAGTGCTTATTGGCCTGAAGGCTAGCTCCGGCTCGCTGCATATGC
TTCACAAAATCAGCCTGCTTTGAAGGATCCACTCTGAGCCTATGCAGACCTCAGTATCGGGAGAACTGGCTCTAACATGTTACTGTTCAGTAAA
CGAGAGGGTCACAGGGTTTTATGTATACTGTGAACACTCTAAGAAATAAAGGAAGGGAGAAAGGGTGGGAGAAGGGGAAGTGAGAAAGAACAGG
ACTCAAGCCCATAGGGAGGTTGTGAGTACTTGCGGAAACTGGGCAGGGATGTGCTGGACGGAAAGGGTTAAAGCCTGAGCCGGCCTCTGAGGGT
GAGGGTACTGAAAGAGAAGTAACTCCCTACAACTTGGAATGTCTTAAGCTGCTACTTTAGTAACAACTTAATGACCTTCAAAAGCTTTCTCTGG
ATTGAAGAGAAACAGAAATTGCAGGTCAGATCTGGGAAGAGCAGAAAAAGCCCAGGAAACATCCTGCCAAAAAGACTTGAGTCAGAGACCCTGG
AAGGCGGAGAGAGGGGCTATCTTTCCGTTGGAGTCATAACAGAAGCAACCTTCCCAGGACCCCCTAGAAAGTCCGGTGCTGAATGGATGGAACT
AGAAAACCTCATCCTGAGTGAAGTAACTGGGAAAGATAACGACGGCCTGTACTCACAAGTGGACGTTAGCCATAAAGTACAGGATAACCATGCT
ATGATCCACAGAGCCAAAGAGGCTAAGTATCAAGGAGGGCCGAAAGGAGGATGCTTGAATCTCACTGAGAAGAGGGAATAAAATAGACATCTGG
GGTGGAGGGAGGGAGGGAAGTGGGTGGAGGCGGGTGGATAGGAAAGCAGGAGGGATCTCAAGTGTGGGGAGGATGGTGGGAGACAGAACTGGGA
TAGAGAACTAGAATCCATGTATACGTGGGGGAGGGGGAAGCATCTCTGGGACAAGGTAGAAATCTAGGACAATGGAAACTCCCAGGAATCTGTG
AGGATGACCCTAGCTAAGACTCCTAGCAGTCAGGGATATGGAACCTGAAACAGGCACTTCCTGACACCAAACGAGACTTCCAATGGAGAGATGG
GAATACCAACCCAGCCACAAAAATCTTTGGCCCTCAATTTGCCCTGCCTATAAGAGGTGGTGGGATAAGGAGGGAGCAGAAAATGAGGAAGTGA
TCAACCAATGACTGGCCCAAATTGAGCCTCATGCCATGGGAGGGAGCCCACCCCTGACGACAGTATTAACAATATTCTGCCGTACTTGCAGACA
GGAGCCTAGCATAGCTGTCACCTGAGTGGCTCTGCCCAGCAGCTGATGAAAACAGATGCAGAGACCCCCCAGTCTAACATTAGACAGAGCTCGG
AGAATCTCACGGCAAAGGGGGAGGAAGGATTGAAAAAAGCCATGTCAAAGACGCCGCAAGAAAACCTACAGAACCAACTAACCTGGGCACAGGG
GGGCTCACAAAGACTGAATCACCAGAGAGCATGCATGGGACAGACCTAGTCCCCCTACACCTTTGTAACAGATGTACAGCTTGGTCCTCGTGTG
GGACTCCCTAACAACAGGAGCAGGAGCTATTTCTGACACTGTTGCCTGCCTTTGGATCCCCATAACTGGACAGCCTTAACAGAAGATGTGATAT
GACATGGCATGTTGATATTCAGAGGTCTCCCTTCCTCTGGGGAAAAAAGGAGGGGCATAGGAGGAGGGGAGGGGAGAAGGAGGGACTGGAAGGA
GAGGGGGTAGGGAAAGCTCTGATCAGATTGTAAAGTAAACGAATCAATAAATGGATGACTAGAATAGAATAGAATAGAATAGAATAGAATATAG
TGCTAGAACAGACCTTGTTCACAGATCCATGTAACACAAGGTCGTTTCAGGGGACTAGAGAGGACAAGAGACTGGCCCACGACCACGTAGCAGA
AGTTGGCTATAAAAGCTCTTTACTGGTCCCTGGTTCTGCTGCTTCCAACCTGTGTGCAGACCATTGCTGCTGGGAAGAGCATGTAGAACCCTAC
CTGCTGAGTGTTTTCTTCTTAAAAATAAAAAAACAAAACAAAAGAATTATTATGCTACACACCAGGGTGCATGCTTTTAATTCCAACTCCAGCC
CTCGGGACACAGAGGCAGGCAGATCTCTGTGAAATATGAATCCAGCCTGGTATACATAGTGAGTTCCAATATAGTCAGGGACACATAGTGAGAC
CTTGTCTCTAAAAACAATAAATAAATAAATAAATAAATAAATAAATAAATACATACATAAATAAATAAATACATAAATAAAAAGATTGTTATTA
TTTTAAATTATGTGTATATGCGTATGGGTATGTGCCCATCTGTAGAGATAATCTCTTGTACACTGTGTAAAAAACAGTCACCTGTCAATTTAAA
AAAACTGACCAATGAGCTTAGGCAGAAAATCAGGAGGTGGGACATCCGGGAGATAGAAGAGGGAGAGGGGGATGCTGGGATAGAGACAGACAGG
AGATTGGCCCCACCCTCAACCCCCAAATGAAGATATAGGCAGCAGCAACAGAGTATCAGGTAACCAGCGACGTGACAGAACTTAGAATAAAATA
ACAGGATGATTAACTTATGAGCTAGTTGGGGGATAACCAAAGCCTATGGCATGAGTGTTTATAAATATATTTGAGTCTCAAGAGTCATTATTCC
AGAAGAGGGACCAGGAGGAAAAAACAGGAGCTCACAGAAAACACACAACTACACCCTTGAGTGGAGGTCCCTGAGGAGGTCAGAAGAGGGTATT
GCATCCCCTGGAGCTGAAGTTACAGGCAGTTGTGAGCCATCAGATGTAGGTGCTGTGACTCAGACTCTGGTCTTCCACAAGAGCAGCGAGCGAG
TGCTCCTGACTACTGAGCGATCTCTCCAGCCCCTGGTTTCCTCCTTTTCCTGACCTGCCTGGGAACCTGAGGTGACTAAGGCAGGATGCTTTTC
CCAAGGACACTTAAGTCAAGTGCTCTGGAAGCTTCCAGCTTCTTCCATGAACAAGGTCAACTTTTCCAAACTGGCGGAGAAAGGCTCCGAGGCT
AATGTTTTTAATGCACGCCTTTGTTTGCTTCTGCCCTGACTGTGGCCTTTTTATATTTTATGGGCTTAATGTTCTAATTTCGTGCTAGGGCCCT
GAGGCAATTCACCGAAGGATGTGATTTTAGAAAATCCAGGAAATAAAGGAATTGTCCGCCAAAGAGGCATCTGACAATGAATTTTATTAGGGAC
AAAGCAAATCAACCTGTCGCCAGACTGAGGTATTAGCATGTCCTGGTTTAATGTAGGTTTGTCTTTTTAATTTAATACTAGCTCTCCGTTCCTG
CCGGGGTCAGCTGGGGATACATACCCGGCTCCTTCTTGGCTCACACTACTGCTTGTAGAAAGCACCCTGGCCTCTTCTCAAGGCACTTGCCAGT
AGTGACCAGAGAGCCTACTGTGCACAGCCTCTGAGCTGGGGGCAGGTCCATGTGCTCAGACAGGGACCACGAGATCCTTTTTAAGGGCCTGTTC
ATTATCGTTTGGAGAATGCCCCAGAGGAGGAGGAACTTGTGGGGAGAGGCTAGTGCGAAAGAGCACGGGTCTTTGAATGCTTGCCTGCTCATTC
CGCACGCTCATCAGCCTACCATACCACATCACATCATACTACACATGACTGTGCAAGTGAAACCCCAGGCTCATCATTCACCCACCTGCCGGCG
TCTACTTTCCAGGTCTCTAGTTGCCCGAACCTATGGTGGCCCTACTCTCTGAAGGCTGTATCCCTAAAACCTGACCCTTTGCATCCCTCTCCTA
CCAATTTAATTCCTAGGGGTGGGGTACTGTGGTTCTTTTGGGAACACCTTAGACCCCACACCCTTTTGTTTCCTGGCGGTTTGACAAAACCCCT
CTGAGTGTTTTTCTTTGCCCGAATCTGGGCAGGTAAGCCCTGCCTTCAGAGAACCCAGCAGACTGGCCACCGTAGCTCTAGCTGCATCATCAAA
TCCATCAGGCCACCCAGGATCCTGTGGGCAAGTTGCTTGACTCTCTGAATGCTGGCTCTCCCTCCTTCCAACTCCTCACCACCTCTTCTTCACT
CTCAGCAGACCTTGCCTCCCACTTGGAAGAGGAAAATAGAAGTTCAAGATGAAATCTCCCTTAACCTCCCACTGCAAACCCACATCCACATGCG
CTCCCATTCTCTCCACGAGCCTGCTTTGGGCTTCTATTGAAGTCCTCCCTCCTCCGTTCTCAGACCAGCCCTTCACCCACACCAATGCCTCCCT
CCCACGTACCCTCAGACATCGCTTCCCAGCTTCCTAAAATGCCCCCCACCACCCCATCTCTCTCCCCTGGATCTAAGTTGATTTTTAAACCAAC
TTCGGTCTCTTCCATCTCAAAACACTCCTCCCCCAATAACACACACACACACACACACACACACACACACACACAGAGTGTTAGTTCTTTTTCT
CCCCTCCACGCAAACCTGCCCAACAGCCCAGCATCTCCCCAGCCCTCCCACACTTGTGGTACCATTGTCCCTGCCCTGTTCTGCATGCAGCACT
TTCTGAGTCAGTCTTCAGTGATAGCAATCAGACCAAGCATGACCCATGTCCATCTCTTCTGGCCTTAGAGCACTTCCCAGCATTTCACATTACT
GAAACTCTGCGCTCAGCTCCCTCGTGAGCCCCCTCTGGCTTTGCTATTCTGCTGCAGCAGGCTTGTCTTTTGAACAGCCCTACCTGTGGGCATG
CCTCAAAAACACCTCTGGTACTACCTTCTTTCTCTCTGTGTGTTCTTGGTGGGCATGGTGTCTACATCTAGAGTCCCAAAGAACTTCTTTCCAT
TGACAAATTTCTTTCTTTTCTCTGACTCAGACCTCTAATTTGTGCCCAAACCAGGTTTCAAAGTACCTACTAGGGACCTGGGACTCTCTGGACA
CTTGCACAGGCCATGTATAATTCATATGGTGTCCTGTCCCTCTAGGGCTCAAATTGCCCCTGCTCTCCAACCAGCCTCAAGCTAAGGAAACCTC
TAAAATGGAGTCTGTCTTGTGCTGTCCCTTTGCGCCCTTCAGTCCCCACATCGGAGTCCTCCTGTGCCCTGCCAGCAAGGCCTTCACCTGGGGT
ATATAGCTCAGTGGGGAGTTCTTGTTTAGCCTTCACAAGGCTCTGTTCTCCAACCCCAGCACCCCATCCTGGGCAGCACACATACACACAGAGG
AAAGCAAACATCTATTTGCCTTTTGCCCTTTCTAGCCACAGGGTGGATGAAGTAGGACAGATTCTGCTTCTCCTCTGCCATTGCCTCCCTCAAC
CCTTCCCCAAGCCCACTTCCTGCTACCTCATCTAAAAGGTTCCGCCTACCTGTCTGCACAGACACCCCAGAAGACACACAAACACCCCAGAAGA
CACACCCTGAACTGCTTCTTGCTTACCATCCCATTCCAGCCGGAGTTAGTGGTGGCTGGGACCAGCTCTGGGGACCTCCATAGGTCATTGTTCT
ATGCCACAGATGTTGCTCCACCCTCTGTTTGCTACCCTGCTGCCTGCACCTCTCACCATTATTGATTTTACCCCTCACTCTACTCTCTGGACAC
TTGCACAGGCCATGTATAATTCATATGGTGTCCTGTCCCTCTAGGGTTCAAATTGCCCCTGCTCTCCAACCAGCCTCAAGCTAAGGAAACCTCT
AAAATGGAGTCTGCCTTGTGTTGTCCCTTTGCGCCCTTCAGTCCCCGAATCGAAGTCCTCCTGTGCCCTGCCAGCAAGGCCTTCACTCGCCTCT
CCCCTTAGTTGTAACCTTTCCCAGGGCAGAACAAGGTTAAGACAGAGTGTCACTGTATAGGTAGCCATGGTTGTCTTGAACTTATTATGTAAAC
CAGATTCGCCTCCAACTCAGAGCAAGCCTCTGGTCTCTGCCTCTGGAGTGCTAGGATTTCAGGCATTGGACACCACCCCCAGCTTCAGCCATAC
CATCTGCCTCAAGATCTTCTACCAGGTGCCCCTCTGACCAGGTGCCTCTGACCAGGTGCCTCTTTTACCAGGTGCCCCTCTGACCAGGATTCTG
CTGATCCAGACTCCCTCACACCTAGCTCAGGTTTCTTCATAAACACCATTTCCTCTGGGAATCCTTCTACTTCCCCTAAGAAGAGAGAGATTCC
TTCTTCCTGTTATTTTACAGTAATTGCTAAGGTACTCAGTTCTATTGCTAAGGAGGTTTCTAAACATTTCTGCTTAGAACGCCCTTCCCCTTCC
TGACAACTCTTGGAGGTAGAAAATGGTAATTATCTCCACTTCTCAGATAAACCAAGGCACAGAGAATGCCTAGAATTGCCAAGCTCACTTAATA
AGAAACAAAGCCAAAATTTAAATCCAGATCGTGTGACTTCAGTGTAGCTTTCAGACACCATACCATTCCACAGGAAGAGGCTATCCACCCTTTG
GTGTACGCTACAGCTATTTGTTTAACAGGTACCTCTCTCATTAGCCTTGGAAGTTCTGCAGACGTATAGACCATGTGGTCTCCAGTAGTAGGTA
TTCAATAAATATCTGCCTGGTGTCTGAATGTAGATTAGTCTCCTGTGTCCTCACACACTACGTACCACCCATTCCTGTCACCTGGTCCTGTGTG
CTTCTCCCTGGAATGTGCATTTGTCACTTTTCTTGTCCCTGTGGCCAAATATCACAAGAACAACTTATATTAGAAAGGTTTATTATGGGGGCTG
GAGGGATGGCTCAGCAGTTAAGAGCACTGACTGCTCTTCCAGAGGTCCTGAGTTCAATTCCCAGCAACCACATGGTGGCTCACAACCATCTGTA
ATGGGATCAGATGCCCTCTTCTGGTGTGTCTGGGGAGAGCAACAGTGTACTCACATATATAAAATAAATAAATAAATCTTTAAAAAAAGAAAGA
GGCCAGGTGGTGGTCGCGCACGCCTTTAATCCCAGCACTTGGGAGGCAGAGGCAGGTGGATTTCTGAGTTCAAGGCCAGCCTGGTCTACAAAGT
GAGTTCCAGGACAGCCAGGGCTACACAGAGAAACCCTGTCTCGAAAAAAAAAAAAAAAAGAAAGAAAGAAAGAAAGAAAGAAAGAAAAGAAAGA
TTTACTATGGCTCACAGTTCACAGGCCATGAAGGAAGGAAAGATGGTGGCAGGAGCTTTGGTTACATGTCAGCTGACCAGAAAACAGAGAAAGA
AAAATACAAGCAGGCCCTACCCAGACATATCCACAGGAGTATCTCTTAGGTAATTCTAAAGTCCAGTCACCACTAAGGATTAATTATCATATGT
GCTTATGTGGGTCCTAAACTTTCGTAGAAATGTATGTTGACACATATTTCTAGAGTGAAAGCTATCAGCAGATTTCCAACCAGTCCTTGAGGAA
CAGCCGCCTCAAATATTCCCAGGGTACTCCCATTCTCTCCTGGTCAGTGCCTCCTCAACAAACACAGTAGCCTACATCCGGAGTCTTTTCTGTA
TTTTGGACTTTCTTCTTCCCAATTCTACAGCCTAGCAGTACAGGGTTCTGTTTGGGGTTCCCTTACCAAATACCAAGGTGGTTCTCACATTCTG
ACTCACTCAGTACCTCCAGCCTCTCTGTGGTGCTTAAAAGTTCCTGCTGTCTTTGGGGCTAAATTTAAATGTCTCTTGGGGGTTGGGTGCTCCT
CCTCCATGCTTCCATGAGCTCTGACTTCTCTCGACAAGGTGCACCAGACATCTGAACTGTAATTACTAGGTTATTCACTGTGTCTCCCACATGG
GTGATTTTTGAGTCAACTGTTTGCTTTGTTTCTCTTTACGTGCTCCTAGCCTAGCTAGGCGCGATCCAAACATATAGAGCTCAGGTAAGGGTTT
GTTTAGAGATTCAACACACTAGTGACATCTCATGCCTCATTCATGGATCCTAAACACTGTGTGAAATGAATACTCGGAAGCTGGGGACAGTGAT
CAGGAAGTTCAAAACTGAATAAAGGCGCCGCAGGCATCAAAAAACCTAGACAGTAAATCCCTTCAAAGGGTAAGAATTAAGATGCAGGAGACTT
TTTTTTTTTTTTTTTTTTTTTAAATCATGGCGGCCCAGAGGGCGGGTCTAGATCGGGACACGCCTTCGAGCCAATGGCAGAGCTAGTTAGCCTT
CGTGGCTCAGCCCTCTTGGATGGGATTGCCCACTCCAGCTGCAAGACTACACTTCCCAGCGTGCCGCGCGGCCCGGTGACGCGGAGCCGGAAGT
GAGGAACATGAGGTTCTAGGGCGCGAGGGAAGCGGCTAGCGGGGGCGAGGGGGCGTGCACGTGAATCCCCTGGCCAGATCTCTGGCATGGCCGA
GGCCAGGAAGCGGCGGGAGCTGCTTCCCCTCATTTACCATCATCTGTTGCAAGCAGGCTACGTCCGCGCGGCGCGGGAAGTAAAGGAGCAGAGC
GGCCAGGTAAGCGTCAGATGGCCCAGTACAAGATCTAGAGAGCAGTAGCCCGCTCCCCTCCCGGGAGACCTGGCCAGCGGGAGTCAGAGATGCG
GGCTAGGGTGGAGGAAGAACATTAAGCTCTGCTCTACTCCCTGCGACTTATAGCCACTTCCCAGGGCTCAGTTTCCCCATGGGTGCAGTGGGCG
GGCCGGAAACTGGTCCAACTAGTGCAGCTTTGTAATTCCCGCCCGGGTTCCCGGTAGCGGCAGGTTTAAGTTTCCCTAGTATCTGGCCACGTGG
CTCTATGCAGTTTCCCTGGGACACCACATGGGAGTCCAGCTGGAGTGGATTGTCACCACTGTTGTTGGGAACACGGAGACGAAGGTCCCCTAAG
TTGATGATCTAGAATGCAGGGCGTGCAGGCAGGCCCTTGGCTGTCTTCGGTTATGACAAGACTTGAACCCGTGGTGCGGTGATTTCAAAGTTCA
TTCATTTGGCAACTTTTGTTTTGTTTTGTTTTTTCGAGACAGGGTTTCTCTGTATAGCCCTGGCTGTCCTGGAACTCACTCTGTAGACAGGCTG
GCCTCGAACTCAGAAATCCGCCTGCCTCTGCCTCCCAAGTGCTGGGATTAAAGGCGTGCGCCACCACTGCCCGGCTGGCAACTTTTAATTTGGC
CCTGTCGTGCGGATGATGAAATGGGACGCGCCAGTTTCAGACTCCTCTGCCCGTCTCACAGCCTAATAGCTGGTACAAAAAAAAAAAAAAAAAA
AAAAAACCCAAAGTGACTGTACAGTTAACATATTAAGCTATAGAACGAGAAATTGGAGATGCTAAAACATGGGTGGAAGAACCTGGGTGGGAAA
TATCCAGGACTGCTTCCCTAAGGATGTAGTAGCTGATCTGAGATCTCGAGGGAGATGGGTATTAGAAGCTCTGATGCGAGGCTTCTGAGTGTCC
TAGCCACCAGAAAGTTTGCTGAGCCTGAAGTTTGAAATACATGACTTGGAATGGAGTCTCCCAAAGCAGACAGACAGGAATGGCTGATGGGTAG
TCTTCCCAGAACTGTGTGTGATTAAGTCACTGGTGAAATGGATATTGACCTGGAGGATCCCGGGTGTTGTAGTGTCTGCCTTTAATCCCAGCAC
TCTAGAGGTAGAAGCAGGTAGATCTATTTTTTTTGGGGGGGGGGGCAGGGGGGTGTTTTGAGACAGGGTTTCTCTGTATAGCTGGCCTTGAACT
CAGAAAGCCACCTGCCTCTGCCTCCCAAGTGCTGGAATTAAAGGCATGCGCAACCACCGCCCAGTTGGTAGATCTCTTAAGTTCAAAGCTAGCC
TGGTCTACATAGTGAGTTCAAGACCAGCCAAGGTTTGCATAATGAGACCCTATCTCAAAAAAAAAAAAAAAACCTTAAGCTGGGGTTGGGTGCA
CCCTGAGTTGGCCCATAACTTTAATCCCATCACTCTGGGGACCAAAAAGGATGGATTTCTGTGAGTTCAAGGCCGGCCTGATCTACATAGTGAG
TTCCAGGACAGCCAAGGCTACACAGAGAAACTCTGTCTTGAAAAACAAAAAAACAGACTTAAAGCCCAAAGTTGCTGGGCCCTTAAGATGTTAT
CTATTGCTTTCTTAATCTTCCTGTATGGTTCTCCATGTCCATTCATACTCAAACTATAAAACAGAGGAAATGGGTTATAGTGCCCATTGTTGTG
GTTCCTGTGCCCAAGCCAGAGTTCCCTGCTCTCACAGAAAAGATGCAAATACAAACAAACAAACATACATACATACTTAAATACATATATGTAT
GTGTATTTAAGTATGTATGTATGTGGCTTAGCATATTGTGGCAGGTGCTGGCCATAATAAGGCTAACAAGTGTCAGCCCTCGGTTATAATTAAG
ATGGTCAGAGAAGCTTTAATGGGGAAGGTGACGTTGGAGTAGAGACTAGAAGGAAATGAGGAGATAGGCTGTGTGGTTGGCAAGAGAGAGAAAG
TCTCAGTAGCAAGAATGGCAAAGCCCTGTTAGGAATTGGTAGCCCAATATACTTTGTCATGACGATACAGTTGGAGCAGGATGGAACTGACTGA
CTGACTGACTGGACTGTGCCCTTCAGACCTGACAGGGTTTCTCTGTATAGCCCTGGCTCTCCTGGAACTCACTCTATAGACCAGGCTGACCTTG
AACTCAGAGATCTGCCTGCCTCTTATCTCCTGGGAGTAGAGGGAGATACTATCCATTTCCCTCTTACATTTATGGTGCTGTGAAGAAGGATCCT
GAAGTTGGCAGAGGTAGAAGATGAGCTGAACGGTCCAATAGGTGCTCTCCCTGACTCCAGGCCAATCTGTTTCGGTGCTAGCATGTTCTTTCCT
TTGCACATGGCTCTTTGGAGTCCATTGGAGCTATTCTTCAGTCTTAATGGTCACACAGAGTGTGTCCTCACCTTAGGCCTGGATTCTACCTGTC
AACCTCTGTTTTAACTCCGACCCTAGCACTCAGTCTCTTTCCAGACATCCACTGGGTTCCGTTATGTCACGTGGAAGCTTTCAGGGTTTGGGCT
GTCTTGTATTTGGGGGAAGATTGACTAGCCAGGAAGCCCTGTCATGTGGCTTCCATCCCTGTACTGTAGTAGGATGCTGGGTAGTGCTAGTTTG
GACTGTTGAGTGCCTACCTCTCAGAGGCGCCCCTTTCCGATGTATCCTATTGAAAGGTTTGTAGCTGGAACTGTTTGTCCTTCTTGACTCTGGA
AGACTGTGAATGGTGCTGGTGAAGCCCAGAGGACCTGGAAGAGCAAACGGCATTTTCCAAAGCACTTGTTGCAGGCCCTGATGTCAGGGAGCAT
CAGGCCCAGGGACGATGCCTATCGTGTTGGGTATTTTTTTTGAAGCTCTCTCTCTCTCTCCATTTCTCTACAGAAGAGTTTCCTGACTCAGCCC
GTCACCCTTCTGGACATCTATACACACTGGCAACAGTAAGTCTGGTGGAATGGAAGGGAGGGAGCAGGGATACCCTAACACGTCATCTTTGCAT
TGAGCTAATATGTGAATGTGTTCTAATGGCCCACTGTGGGCATACTTGTCACCTTCATTTGGTCAGTTCCACCTCCCCCACTTTCCATGTGTGT
TCTGTTTTAAAGAGAGGATCTCACTATGTAGATCAGGCTACCCTTGAACTCACGGAGATCCTGTTGCTGCTGCTGCCTCTTGCATGCTGGGACA
ATTGGAGTGTGTCTGGGATTAAAGGCGCACGCCTTGGGAGGCAGAGGCAGCCTGGTTGGTCTACAAAGTGAGTTCCAGGACAGCCAGGGCTACA
CACAGAAACCCTGTCTCAAAAAAACAAAACAAAATAAAACAAAACAAAAAACCAAATGCTCACACCTTTAATCCCAGCACTCCGGGGATATTGA
GGTAGTCAGGTCTCTGAGTTCAAGTCCAGCCTCGTCTACAGAGTGAGTTCTAAGAGTCTCAAGAAATAAAGGAAAAGAAATGGAACAGCTGGGT
TTTTATTTTTTTATTTTTGTTTTGTTTTGTCTTTTCAAGACAGAGTCCCTGGGTAGCCCTGGCTGTCCTGGAACTCCCTCTGTAGACCAGGAAT
TCAAAGATCCACCAGCCTCTGCCACCCGAGTGCTGGGACTAAAAGGCATGAGCCTGCACGGCCTGGCTTGTTTCTTCTAGGTTGTAATCAATGC
GGCAGGCACAAAGTCAAGGAGATGCTAGTGGTAGTGAGAACTAGTGAAGGCAGGAGCCTGGCGCCCAACCTGAGCGCTTACCATCTGAGCTGCT
GAAAAGTGAGGTTGGTAGTGACAGTTCTATCATGGCAGCACCGAAGGCAGGGGATGCCAGGCTCCGGAGGAGTACTCGGGTCAGTGAAAATATT
GCCCACCCTCCCTCCATACTTTTCATGCTCCTCTGCTGCTCGACAAGCTCGGAGCACGGGTGCGTCCTTGCCCAGGTGTGTGGGTGTTGCTTTC
TGTGCTTCCTTCCCCAGCAAACTGTTGGAGCTGTGCCACACACAGTTTCCTGCCTCTGCCCCCTCTGTGGGTGAGGGGTACAGAGTGGGCCAAA
GATCTCACCCACTGAGCCAGTCCAGAGCAGCGATTACCTATCAAGTCAAACCCAGTAAACGAGCTACATGACCTTGAGCAACGTTTGTGACCTC
CTAGAATAACATCATCTGTAAAGTGAACTCTATCATCACGTATTGGGAAGAAGTTCAAACACAGTGATCTATTTGATAACAGTCCTAAAACTGG
CCAGTGAGATCTGTCCTGGCACTGGTCCTCAGATACTACATTCTTGATGTTACAGAGAACAAGCAAGAAGGGGGGGAAAAAAAATCCCTGTTCT
TGTGAAATTCACATTCTGTTGGGGGATGGGGTGGGGGGGTGGAACAAACTTATACCTGTAATAAAGCCTCTTCTAACAGCTTCCTGGCAGGTGG
CCTTCCTAGGAAGAAGGAACCTGGGTAACACAGCCCAAACTGTGGACAGCAGTGTCAAACCCAGCTAGGAAGAATGGGGCTTAATGACTTCCTG
CCGGTAGAAGGGTGGAATCAAAGTTCAAGGGACTAAGCTGTCTGAAGACTTACAGCACGCGTGCGCGCACACACACACACACACACACACACAC
ACACTCCCCACTCTGCTTCCTTCCAGACTCCAGTTCGATGGTGGTTCCTTTCTCTAGGTGTGTGCCAGATACTGGGTGGGATGCTGTATGCTCT
CTGGGGGCCTGTTTGTATAGTAGTGGAGTCGTCCAGAAATCAACCAGAGAAGGAAACGAAACTGAACGCCAGGGCGGAGTGCTGGGGGGAGGGG
GGAAGGGGTGTCAGCAAAGATGCTTCACGCCAAACTCGAGAGTAACGAGAGTGCTCCTTCCTCTTTGATGACTCTAGCTTGTGTCAAGTTAACT
GGCAAAACCAGTCAGAGCTGGGCGTGGTGGCGCACGCCTTTAATCCCAGCACTCGGGAGGCAGAGGCAGGCGGATTTCTGAGTTCGAGGCCAGC
CTGGTCTACAAAGTGAGTGCCAGGACAGCCAGGGCTACACAGAGAAAACCTGTCTCGAAAACCAAAAAAAAAAAAAAAAAAACCCAGTCAGGAC
AGCACCACAAGAGAAGGACGCAGTTCTAGGGTCGGGAGAACCTGGATGGTTCTGTGCAGCCTATGGGCTGTGGCCAGTCAGCCGATAGTAGTGA
GTGAGCTGCTGCCGTCCTGTCACCTCGTCGGCTGCTTGGGACCCTGAGACTGGTTAAGGTGCATTAGGAAGCGGGTGAAGAACAGATCATTTCT
GCACTGTGTCGGCGCCACTGTTTAAACTCTGTGTGCCCATTTCCTTTGAAAGACTGAATAGGTAAACAGGTATCTTTCCAAACCTCATGGATTT
GTCCCATCTCCCCTGCCCTGCAGGACCTCAGAGCTTGGCCAGAAGCAGAAGGCAGAGGATGATGAGACCCTTCAGGCTAAGAAGTCTCGAGTGT
CGGATCCTGTTAGCAGCTCAGAGAGCTCGGATCAGGAGAAGGAAGAGGAGGCAGCAACCGAAAGGGCCAAAGCCAGTAAGAGCCCTCCTTGCTG
TGTGAGATTGGGACCCGCCTTGGCAGTTGCGGGCTTCTAGTCTGCTCTACCGGGGTGTCTGCCTCTGAATCATTGTTTAGTGTCAAACAAGAAA
AGGTGGGGCTTCTCAGAGGGAGGTTGTAGTACCTGGGAGGCCTTCCTTTGGCAGGCATTTTAACCCCTGAGGGCAGGCATTCTAATCCTCTGAG
GATCCAAGGATGAATCCTAAGATCCCAGGAGGCTGGGTAAAGTGGCGCACACTTTTAATCTCAGCACAGAGACAAGTGAATCTGAGAATTTGAG
GCCAGCCTGGTCAACATAGTAAGTTCCAGGCCAACCAGGGCTACAAAGTGAGAACCTGTCTCCCCCCCCAATCTCCCTTCCCCCAAACAGGATG
GCCCACACAGTCAGTTGCTCAGTTCACTTCACAAGGACTGTTTCCACAGCTCCTGGATGCTGCTGCTAGAGATTGGCTAGGCATGGGGGGGTGG
GGGGGAGGCATTTGTGCAGGTGTTGAGTCCATTTCAGAAGGTTGATAGTTCTGGCTTCTGTGTGGCCCTGGGGCTGCTTTCAGACTTGCTGGTT
GTTTTAGTTACTGTTCTGCTGCTATGAGGAGGCACCGTGAGCAAAGCAACTGACATAAAAGGAAGCATTTAGCTGAAGGGCTGGCTTACAGTGT
CAGGGGAGTTCATCCATGATCCCCATAGCAGCAGCCAGGCAGGCATGGCACCGTAGCAGTTCCTGTGAGCTTACATCCTGATCTGTAGACAGTG
AACAGAAAGCTGGAGACTGGGCTTGGTGTAGGCCTTTGAGACCTGGAAGCGCACCCCCGTGATACACCTCCATCCACCAACCAGGAACCAAACA
TTCAAATATAGTCTCATTCAAACCACCATACTAATTGTCCTCTTATTTTGGCGGTATGTGGTGGGTCTGGTAGCTATGTGGCCTCTTCTCTCCT
GACAGAAGCAGCTAGAATGCTACACCCAGCAACTGGCTGTGTCTCTGTGTCCCACAAATATCATATTGCTTATACAGAAGACAGTTTCTTGATT
TTTAAGTAGGTTTTCTGCTTATAAAAGAAGTGCAGGATTTATTATAAAATGTTCATTACAGAACATTTGGAAGTTGTTTTAAGAAAAGGCAGGG
GAGAAGTCACCCACATTCTTGACATCTATACCCCCACAATCTTGTCTTTCATTATTCCTCCTGTGTATGTTCTGTCAGATGCTCAAGAGGACAT
TTTCGGTGCATATTTTATATCTTAATCTTAAATTATAAATGCAAAGCAGGTCCAGTTTATAGACAGAAGCACAGAGAAGGGAAATGTAAGAATC
CTTGTTGCCAGCCCCATCACTTCCTACCCATGTCTCTTGGTCGGCAGCCCATAAACCTGCCCTGTTGCTTCCAGTCAAACAGGAGTCCAGCTGT
GTGCATGTGGACTGACCCACAGCGGGCTTCAAACAGAAATGGCAAATGCTTGTGCTATTAGGTATCTGATAGTAATCGGTTTTTTTAAAAATAC
CTTACTCTTTAACAGTTTCATACAGTATGTGTTGATCATGCCCATGCCCAGCTCCCCCGGGGCACGCCCCCAGTATGTCCCCTCTCCTTAGTAC
CCTCTTTTTGTTTCCCGAGTTTGTTTTTTTATTGTTGCAATCAACTGGCTGGATCCAGTTAGTGCTGCCTGCAGGGTGTTGACTGATCCTGTGT
AGGTCACCGGGCTACATCAGGTTCATGAGTATGACCTTGTCGTGACCATGTTTCATCAGCTTTGCATTCTTTTGTTTTTGTTTTTGTTTTTTTG
TTGTTGTTATTGTTTTGGGTTTTTTTTTGTTTTTCGAGACAGGATTTCTCTATGTAGCCCTAGCTGTCCTGGAACTCACTCTGTAGACCAGGCC
GGCCTCTAACTCAGAAATCCACCTGCCTCTGCCTCCCAAGTGCTGGGATTAAAGGTGTGCACCACCACTGCCCAGCCAGCTCTGCATTCTTTTT
TTTTTTTTAATATTTTTTTTATTAGGTATTTTCCTCATTTACATTTCCAATGCTATCCCAAAAGTCCCCCATACCCTTCCCCCCACTCCCCTAC
CCACCCACTCCCACTTTTTGGCCCTGGCGCAGCTCTGCATTCTTAACACCATCTATTCTATAATGCTCCCTGAGCCATGTAGGCTTGATAAATG
TCCCACTGAGGGCTAAGAGCTCAGGTGTCACTCAGTCTCAACTTTGACAAGTTAGGAGTCTCTGCATTGCTATTGCAGAAAGGTGAATGTGTGT
GCATGCGTGTGTTTGAAATGTTCTCATGGCAAACTGACAGTAGTAAGTCCCATCACTTATACCCCCAGCCTACAGTCTTGAGCTAGTAGTCACT
TTTAAGAATTATTTATTTTATATATATATATATATATATATATATANNNNNNNNNNNNNNNNNNNNNNNNNNNNNNNNNNNNNNNNNNNNNNNN
NNNNNNNNNNNNNNNNNNNNNNNNNNNNNNNNNNNNCCCGGTATCTAGACACACCAGAAGAGGGCATCGGATCCCATTAACAGATGGTTGTGAG
CCACCATGTGGTTGCTGGGAATTGAACTCAGGACCTCTGGAAGAGCAGTCAGTGCTCTTAACCATTAAGCCATCTCTCCAGCCCTATAATTGAC
TTTTTACATTTTGGGTTTTTGTTGTTTCCCCCTAGTTCTTCTTGCGACAGGGTCCTGATGTGTACCCCAGACTGGCCTCAAACTCAGGGACGTC
CTGCCTCAGCTTTCTGGGTGTCTGGCTTAATTTAAGAGTCCTCAGAAGTATTTATGGGTAACATGACAGGGTGTGAAGTTCTTCAAAATAATGT
ATGATGGGGAAGGGAAAATGAGACATTGGGTGAACTGAGGTTGGCTACGGGTTGATATAAAGTTACAATAACAGCTGAGGCTGCATATCATACA
TACACACACACACACAAAAGCTGGAGTTAGGTGACTTTGGTGTACTTCTGAATATTTTGGAAACTTTGCTCATGTGCAGAAAAGGGAGTGTCAA
AGAGCCCCGAAATGAATTCAGTCCTGCCGTTCATTTCAACACCTGTCTCTTTAGTGATAACTTCTTCCCCTGAATGCCTGAGATCCAAAGCACC
GTTTCTTAAAACCCTCGAGAGTCTGAATCTCAGTGTCTCCTACTAGTCCCAGGCTCCTGGGTCGGCACAATTGACGACCACATAGCTTCCTCTA
TGGCCTGGGGCTGGCTGCTTGACCACTGCCCTGCCTGCCAGTGACTTATCAGGACTGATGATGGTTGGCACCCAACCCAAAGAGTAGTCACCAG
AGTTGGGCATGTCTTGTGGTGGTGGGTGGTAAGATTATAAGCCTGTCCTTTCCCTCTGGGGGCCTCCAGATCTCATGCCACAGAACAGCTGGGA
GTCCCTCGGGGATGCAGGCACAGTAACCACCATGGGTTCTGGGCGAGGCCTTCTCATGGTTCTGGGCTAGGCCTCAGGTCCTCATACTAGCACC
AAGCAACTAGTACCTGTAGGACCATTAGGTGGTGTAAAGTCTGCCCATCTTCTTAGATGAGAAGAGCCCACCTTGCTCACAGCGTTTGTGTCTT
GCAGCCCCAAGACCGACACCTGTCAATTCTGCAACCGCAGCTTTGCCATCAAAAGTAAAAGAAAAGGTGAGTTTATTTCCGAGTTACTCATTAC
CTGTGGAAGCTGTATCCAGTGGAGCATAGTCTGCCGTGTCAGGATAGGGTCGTGTGGTTCAGCGTGTGTCTCTAGGTGTCTGTGACTTCATCTA
GCTATGCCTCGGGTGCCTTTTCTGTGAAACAGAAGTGATGCCTCAAACCACACACTTGGGCAAGTGCCTTCCTCTGTCGTCCTTGCACCCACTC
ACCGGTTGTATAAACAGCTCCCCTCCAGTCCCTGGTGTGCTACTAGGCCGTCAGTACCATCATAAGGAATGCACTGTACACATTTTCTAGATCA
AAAAATACTTCAAAAATGTCATTTTCCAGTAATCCATACACACTGTTGCAGACACATAAATGATGTGTCACCCGAACCAGGCGATAGCTATCGT
ATGTAAAAGCAATGATGAAATAATAATAGTATCACAGTGAACTATAGCTTCATGTAAAGTAGGAACTACTCTGACCCAGAAGCAGTAATGCTCC
CTTAATGCTTGTGTGGAAGGCATCTTTGTCTTGCTGTAGAGATGAGATGACAAACCAGTGGACTGTGAAAGGCCAGAGCTGGCTGCTCTTAGTC
TGTGGGAGAGGTGGGACACTCAGAGAGCTCGGAAGTCGCCTTGTAGGACCCAGTGTCCTCCTGATGCTTTTCACATGTCCTGTCATGACAGAGT
GCTCATCCCTCCCATACTCTGTAGGGAAAGACCAAGACAGCCAACAAGACGGTGAACTCTGTGTCGCACCCTGGGTCCGGAAAGACGGTGGTCC
ACCTGCTCTCTGGGAAGTCACCCAAAAAGTCAGCAGAGCCCTTGGCAAACACTGTCTTGGCCTCAGAAACTGAGGAGGAGGGCAATGCCCAAGC
CCTCGGACCCACTGCCAAGTCTGGTGAGACCCCCAGAGCCTGGGGCAAGGGGTCAGGACTGAACACTGTGGAGAGACATTCTGAGTGTGGGTTT
GTCTCTGACAGCCAGGGTCTTACAACAGCCATTTCCACCTTTTCTGACTTCCTAGACTATTTTAAATTTACTTTTATTTTATATGCATCGGTGA
TTTGCCTGCATGTACATCTGTATGAGATATCACACTTTGTAGTTATAGACAGTTGTGAACTGCCATGTAGGTGCTGGGAGTTGAACCCAGGTCC
TCTGGAAGAGGAGTCAGTGCTCTTAACCTCTAAGCCATCTTTCCAGCCCGCTATATGGGCTTTTTAGAGGGTGGCGTTTCCGGCCTTCTCTCTC
TGGTGGATTATAGGGAACACTGTGGTCACCTGGAAACTAAATGAAAAGGTGTAGGTCTTGATGCAAAATAGGCCTTTGCCCAGCTTGTCCCCCA
TTCCCCAACTTGATAGCAAATAGTGACTTGATTAACTAGCAGGTACTTCTAGCCTTTCCAAGACCTCAGAGAGGCCTGCAAAAATAGGTAGTGA
GGGTCCCTGTTCCAGCTAGGGGACTTTGGGTCAGAGCAGATAGCAAGCCCTGTACAGTTGAGAAGCATGTGCTACCTAGGTTTGACTAACAACC
TGCATGGAGGCTCTGGGAGGGGAGAGGACTGTGGGAAGGATCCTTAGTCCCCTGTGTCCCCCCCCTCAGGAACGGTGTCAGCGGGCCAAGGCAG
CAGTTCCAGTGAAGATTCCTCCATCTCAAGCGATGAGACAGATGTCGAGGTAATTGCCACCCATCCCCAGGGATTGATCTCTTCAAGCTTCTGA
CCCTCTCTTGCCAGTGTTCAGATCCCAGTGGCCAGTCATCAGCCCAGCAGTTGTGTCCGACAGCTGCATGGTTACAGACAGGGCTAGGCAGTTG
GAGAATGAGGACCTCAGTACTGCTCTGGATATGCTGGGTATCAGAGTCCAGTGCTGGGAGAGGAAGACACGGGAGCAGAGACCTGAGTAGGAGT
AACTGGGACATGTGGCCAGGGGTAGGACTGCCTCGAGGATTCTGAGCCCAGTGGTGAATGTGCGTTTGGGGATAGAATTGGATGGGATCCTTGG
TCTGCAGCCTTGTCTCTTTGTAGGATCGTTGGGGGGGTGGGGGGTCATCACAGGGTTTTATGCACCGAAGTGAGGTGGCCTGGCTCGCATCTCA
AAAAGATTGCTGTAGCGTGGCCTGGAGGAAGATTTAGAGTAAAATTAGTGAGGCCCACTGGCAGACTGTCAGGTAACAGCCACAGGTGCCAGTG
GCAGGAACCAGTATGCCCCATATGTATGACATATATTATTTGGCATAATAACCAACCTCTACCCCAGTACAAAGCCACAAGCTTATAAAAACAA
AAGACAAAAACCATGAGGGTGTTTATATTCCAGTTGTTGGATCTTGGGCATGAACTTTGTAAATCACAAGACTGTATCACACTTTCAAAAGGTT
GGACATTTAAGGGGGTAGCAGGAGCCAGGGATAGACTGTGCCTACGCAGCTGCCCCACCAAACCCAGTGGCCCTGTGTAGCCCGAGCCATCCCC
TTCTGTTCAAGACTTGACCACAGCTGTTCCCTGCCTGCCGTCTCTCTGTTGGGCCCTGAGTGTAATAAGGCCCACGGGATTCCTCTGTGTCCCT
TTACTCCAAGCCTTGCTCCCACAAATCCAGCTCCTACGTATACGTATACAGCACGTGTACAGCACTGGCAACAGGAAACCAGCGCGGAGCTCCT
GCCGCTGCGTTTGAAAGAGGGTGGTGCCATAGGCTGGTGTGGGGCGTGAGGGGCAGCGTGAAACAGGCGCTGACTGGCCTGCTGGCTACCTCAG
GCCTGTCGGGATTTTGGAATGCTAAGGAAGTTCCAGGAAGAAGTTCAAGTCAGTGCAGATCCAGGCTCCTCTGTCCGGAAAGTACCTCAGTCAG
ATGCTTTTTAGTTGAGTGGCCCCTCAGGATGGACCCTCTTATAAACATGTCAGTGGGCAGGCAGGTGCGGTGGCCGAGTTCTGCCCCAAAGGTA
GCCAGGGGTCCTTATAAGCCTTTTGTGCTGCCCTTAAATGATCTGTGCCTTTTTAGGAGAAGGGTGTAAGATTCTGTCTGCCTCTGGGAGATGG
AAAGGAACATGCTTTTAAGAAGGGGCTTGTATTCCTTTTCTGTTGCTAAGAACAGCCCCCATAGGCTCATCTATCTGAATGCTTGGTCACCAGG
GAGTGGAACTATTTGAAAGGATTACAAGGGTTAGGAGGTGTGGCCTTGTTGGAGAAACCCCATCACCGCTGGGGGTGGGCTCTGAGGTCTCAAA
GGTTTCCAAAGTCCTTTTGTCTTTGTCTACCTGTGGATCAGGCTGCAGCTCCCAGCTACTTCTCCAGCAACACGTTCTTCCTTTTAAACGACCA
CCTTGGTCACGGCGTCTCTGCACAGCAATAGGGCAGTGACTCAGACGCCAAAGCAAGTTTTTTAAGAAAAAAGTTTAGTTGAACTTATGGTTTC
AAAGGGTTAAGAGTTTGTGACGGCAGAGCAAAGGCACAAGAACTGGAACCGCTCGGAGCTCGAACCCCGATTCACAAGCAGGAGGCAGTGAGAA
AACGCAGGGCATAAGCCTACCCCAGTGACACACGCCCTCCAGCGGGGCAAACACTTCTCGTAGTTGGGAGCCTATGGGGCCGTTCCCATTCAGA
TTGGCAGAGAGCTGACAGCAGAGGGGGAAGGCTCTTCATGTGCTGCATCTCTTGCCTGCTTCTAGGTGAAATCTCCAGCAAAACCAGCCCAGGC
CAAAGCTTCAGCAGCCCCTGCCAAGGATCCTCCAGCAAGAACAGCCCCAGGCCCTACCAAGTTAGGGAATGTGGCGCCCACACCTGCTAAACCA
GCCAGGGCGGCAGCGGCAGCTGCTGCTGCTGCTGTGGCTGCTGCTGCTGCTGCAGCAGCAGAAGAGTCTGAGAGCAGTGAGGAGGACTCAGACA
GTGAGGACGAGGCCCCTGCTGGTCTGCCCAGCCAGGTGAGTCCAGGGGCTGCTATCCGTGACCCGCTGCCTGTAGTTCCCCCCCCCCCCATGCT
GGCAAAGCCACTGTGTGATGGAGAACTCCTGAAGAGGGCCCATTCTAGAGAGAGAAGCAATGGCAACTGGCCAGGGGGCTTCGGTGCCTGTGGA
AACGTCCACTGGGCCCACACCGCAGCCAGAGTGGCTGATGCCTTTGGGGAGTTGGGGGGTTGTCCTGCACCTACTCTGGTGATGATGTTAATTT
TCTTGGTTTTCGTGTGTGGTCAGTGTAGCTGCCCTCAGCACAGCTGCTCGGAAGAGACCTGAGGCTTTCTTCATTCTGAGTCGGTTTCCCTATA
GCCCAAGCTGGCCTCTAACGCATTCTCTTCCTGCCTGAGCGCCCCAAGTTTGGGGGCTACAGGCACGTGCTATCTTGCTGTCTTAAGTTTTCTA
TCGCTTCAAAGAGTCGTCATCACAAAGCAACTTATAAAAGCGAGAATTCAAATGGGTGCTTGCAGTTCCATGGCCGTCCTGACTGAGAACATGG
TGTCAGCCAGGCATGGCACTGGAACAGTAGCTAAAAGCCTTAAAACTGGTCCACAGGCAGAGAGAACGCTACCTTCAAAGCCATTTAGTACACC
TTCTCTAATAAAGCCAGGCCTTCCAAACGCTCCCACCGATGGAAGGGGTGGGGATGGGGGAGACACAGCAAGTATTCAGATATCACATGTGTCC
CAGTGAGTCCTGAGAATGTTCAAGGGCTTGGTTCTCTCTTTAGCCAAACCAGACCCTGAGCCGGCAACCACCAGAGAGAATCTCTTTTGTTCCT
CTTCTGGGGCCTTTCCCATGGAGTGTAATTCAGATAGCAGTCTCACAGTCCTGCACACATGCTGGGCGGAGCAGAAAGGGCTGGAAAGCTTTTA
GAGGTAGAACAAGAAGAAGTGAAGGGGTCTGTATCCAGGACTGGGGCAGGGGTAGGGGGTGGGGCGGGGGCATGGCAGTTATTAAAATGTTAAA
GAAGAGTTGGTTGACAACATGAAGTGGACAAGGAAGGACCCAGAAGGAGATGTGACCACAGACCTATAGTTGAAGGGTGTAGAGGATATAGTTG
GCTGCTGAAGTTCTGTGGCAATAACTTGGGACCCAGCCTACTTGGGGCTGCATTGTGGGTATTGGTTCATGTCTGAAGAGAGGAGCCAAATGCA
GCTTGTGTACAATTACTGCCCCTCCACACAGAAGGCCTTAGAAGGTCCAAAGCCATTTATTTTCTAGGAAGCTTAGGGCAGAGCGGACAAGGCC
GTCATGAGAAGACTGTAGAAGCTTGTAGCCACCCTACCAAACAGGGAGGCAGTGGTGAGCCTTTGGCTGAGAACAGTGAGGAGAATGGATAAAG
AGGAGGCCAGTGAAGGGACTGCATCTAGAACAGTAGTTCTCAGCCTCCCTCATGCTGTGAGCCAATACAGTTCCTCATGTTGTGGGGACCCCCA
ACCACAAAATCATTTCATGGCTACTTCATAACTGTAATTTTGCTACTGCTATGAATCAAAATGTAAATATCTGGTATGCAAGATATCTGATGTG
GGTCGTGACCCACAGGATGAAGACAGCTGATCTAGGAGTCCTGTCTACCACTGGAAGCCCTGGGGAGTTTGAGCCTTTTTCCATGCTCAGGCTG
TTCTTGAAGAACTTTGCATACACCCCTTCTCTGCCTTGAGCATTGCTGGCTAGTTAATGAGCCGGACAGTCTCACAAGCCAGTGCTTGTACCCA
CAGAACAGACTATTCCAGCTAAGGATTTAACCATTTACGTGCTAGGGCTCTGTTCAGGGCCAGAATGAAGGCCACTATATGGTGTCACTGGGTG
CTGAGGAGGTCAGTGCTCAGGAAGCCGAGGGCCAGCCGGGGTGGCTGGGGCACCAGTGAAGATGAGAAAGGAAAGTGTTATCTCCAGAACCTCT
GGGCTTCTTCCTGATGGCATCACCTAAGGACTGAAAAAGAAGAACGGGCTATTCTTTCTCTTCACCAGGTAAAAGCCTCTGGAAAAGGTCCCCA
TGTCAGAGCCGACTCGGTGTCTGCCAAGGGGATCTCTGGGAAAGGGCCCATCTTAGCAACCCCAGGGAAGACTGGGCCTGCAGCCACCCAGGCC
AAGGCAGAAAGGCCAGAGAAGGACTCGGAAACCAGCAGTGAGGACGATTCTGATAGTGAGGATGAAATGCCAGTCACTGTGAATACTCCTCAGG
TAAAGCCTGGGGGGAGAAGGGAGGCATTATAGCCCAGACCTGAAGGCTGCTAACCTGTCTCCTCAGCTGGTCCCAGCCCCTGTCCCTTCTCCTC
AGCTGGGCCCAGCCCCTGTCCCTTCTCAGCTGTGCCCAGCCCCTGTCCCTTCTCAGCTGGTCCTAACCCCGTCCCTTCTCTTCAGCTGGGCCCA
GCCCCTGTCCCTTCTCCTCAGCTGTGCCCAGCCCCTGTCCCTTCTCCTCAGCTGGTCCTAACCCCTGTCCCTTCTCCTCAGCTGGGCCCAGCCC
CTGTCCCTTCTCCTCAACTGGTCCTAACCCCTGTCCCTTCTCCTCAGCTGGGCCCAACCCCTGTCCCTTCTCAGTTAGTCCCAGCTCCCATCCC
TTCTCCTCAGCAGGTCCCAGCCCCCATCCCTTTTTACTGGTAAGGCTCCCTGCTCCTGTCAGGTCTCTGCCCACATCTGCCTAATGATTTACTC
CTCAATTTCTCATACCAGGCAAGGACTTCTGGGAAGAGCCCTCGGGCCAGAGGTACCTCAGCCCCCGCCAAGGAGTCATCCCAGAAAGGGGCTC
CTGCAGTCACCCCTGGAAAGGCAAGGCCTGTGGCAGCCCAGGCAGGGAAACCAGAAGCCAAGAGCAGTGAGGAGTCAGAGAGTGACAGTGGGGA
GACACCAGCTGCTGCGACTCTGACCACGAGTCCTGCCAAGGTGAGGCCAGGTGTGCCTTGTGCTCTGCCTAGAAAGGAAGGCCTCCTTCTTCAA
AACCAGAGCACGTCCCCTGATCTTGCCCTCTGTCACTCCCAGGTGAAACCTTTGGGGAAGAGCTCCCAGGTCAGACCTGTTTCCACCGTCACCC
CGGGGTCATCGGGAAAAGGTGCCAACCTGCCCTGCCCTGGGAAGGTGGGGTCAGCAGCTCTCAGGGTCCAAATGGTAAAGAAAGAAGATGTCTC
GGAGAGCAGCAGTGCAGAGCTGGACAGTGACGGGCCTGGGAGCCCAGCCAAGGTGAGTCCTCAGAGGTCTCAGCCCGGGAGCCTACATGGCCCT
ACACCCCAGGTCTGTCGTCTGGGTCTCTAATTGCTGCCTGTCTCCTACCTGGGGCTCCCACTGATCTCATTCCAGGCAAAGGCCTCCCTTGCGC
TCCCTCAGAAGGTGAGGCCTGTGGCCACCCAGGTCAAGACTGACAGGGGCAAAGGCCACTCAGGGAGCAGTGAGGAGTCATCTGACAGCGAAGA
GGAGGCAGCACCAGCAGCCTCTGCTGCTCAGGTGGGGACCAGGGAAGAGAGGCAGTCTGGCTGTCCTGGCCCGCCCCAGTCAGAAACCCCACCT
CTCAGAATTAGATGGGTTCTGGTTAGTAAGGAGACCAGGGGGAGCTGCATCTCAGAACGAATTAGTCCAGGAGTCTCCCCCAACCTCCATCTGC
ACTTTCTCCACAGGCTAAGCCAGCTCTGGAAAAGCAGATGAAAGCTTCCTCTAGGAAAGGCACGCCTGCATCCGCAACAGGAGCGAGCACCTCG
TCCCATTGTAAGGCAGGAGCGGTGACCTCTTCAGCCAGCCTGTCATCCCCAGCTCTGGCCAAGGGCACCCAGAGGTCAGATGTGGACTCTTCCA
GTGAGTCTGAGTCAGAAGGAGCTGCTCCCAGCACCCCCAGGGTACAGGTAAGGGCTGGATTTACCCAGGTAGGCTCACTCCCAGCCCCCATGGG
GCATCTCTGGCCCTTGAGAAACTCTTTCTTCTCCTTGACTTTCTAGGGGAAGTCTGGGGGTAAGGGCCTCCAAGGGAAAGCTGCCTTGGGGCAA
GGGGTGGCCCCAGTGCACACTCAGAAGACAGGGCCTTCGGTCAAAGCTATGGCTCAGGAAGACTCAGAGAGCCTCGAGGAGGACTCCAGCAGTG
AGGAAGAGGATGAGACCCCAGCACAGGTGAGGTAGGGAGGAAGCAGCCTGAACCAAGACTGGGGATGAGCTCAGTGGAGGCTGCCCTTGGCCCT
AGGAGCACAGAATCCTGGGACTCAGGCTCAACTAAGTCTCACGTTCTCACACGCTCTTGTTTCCTGAGCCCCCTGGCTCTAGGTTCCTACACAT
CTGGGTGTGCCACGCATTCTGCCCACTGCCACTCTAGACAGAAGTCTGTGGCTTTGGCCTTGGGGAAGTTGCCACCTGGGGCAAGGTGTGATAT
AGTACACACCGTTACAGCAGGATAGGGATGTGCAGTAGGGCTAGCCAAGCATGGTACCCGAGATATTTTAAGAGGAGTGTCATGTCGGTGAGGA
ACTGAAAGTCGTCTGGTGAGGTAGAGGGTAGACTCTTTGCAGAAGCCAAGAAAGTTGGCTCGGGTCGCACCACAAGCAGGATTAAATGGCAGAG
GCCAAGAGCCAGGTTACCTGGGCTCAAGTCCAGTTCTATTCCTGGGGCCTTGGCTACAGTCCCATCTGTCATCAATCCACCCTTGTGGGTATGG
GTAAGGCCTCGCCTGCCGGAAGCCCGGGGACCTGCCTTCCTGGCCGTGGGCTAGGCTGCTGGGGGCAGAGTTGCAAGGCACACCACCTGGCCCG
AGAGAGCAGGAAATGAAAAGGAGCTTGTCACGGTCTGCGCTATGGGGGACCTTCCTTAAGTCCTAACTCTGTCGTACACATGAGAGGCATTGGG
AGTTGGAGCTACTTTGAAACTAACTAGATGGTGATATCTGAGTGAGTCGGGGCCTTTCTCAGGTCCACACTCACCGAATTGCACTCAGAGCCTA
TCAGTGTCTGTCTTACTCTGGCTGAGGCCATAGCTCCTAGCTGGGTGGTATAGTGTTTGCCTACCATGCACCAGGCCCTGGGTTCCATCTCCGG
CACAGCACTGACTCCGTAATGTGGGTTTGTACAAGCTGAACACCCTGACAGGCATGGGGGGGGGGGTGAGATTAAGCAGAAGCAATGACTCTAT
CGTGTTCTGTTAACCTGCCCAGTTCCTAGTTCCTGGGCTGAGACGGATGCTTGAGCTCTGCATTTCAGGGAGAAGAAAGCACAGATTGACGATT
CTATATGGCACTCAGCTTCCATTCTGTAGAGTAGAATTTAATGACAGACACGGAGTCTAGTTGAAAGGGAACCTAAAAAGCAGTTGTGTTTGGG
TAGGAGTATATTTAAAAGGCTTTACAGTGCTGAATTTGAGTGTGTGTTAGTGCCAGTTAGACTACTTAAACACGTAGTCTGTTTAATCTTTGCA
GAAATCCCATAGATAATGTTTTATTTTTACACTCCTTTTATAGAGATGAAAGTGAGGCCTGGTAAGATTAAGTGGCTTCTTCTTGGTTGTGTTT
TGTTTTTGGAATCAGGTCTCACTGCATAGCTCTGGCTGTCATGGAACTTGCTCTGTAGACCAGGCTGGCCTCAGACTCACAGAGCTATGACTAT
CCCTGCCTACCAAGTGCTGGTTAAAGGTGTGCACTGCCATACCTGGCATCAGGGACTTCCTTTTTTTTTTTTTTTTTTTTCTGAAACAGGGTTT
CTCTGTGTAGCCCTGGCTGTCCTGGAGCTCACTCTGTAGCCCAGGCTAGCCTCGAACCCAGAAATCCTCCTGCCTCTGCCTCCCAAGTGCTGGG
ATTAAAGGTGGGCACCACCATCGCCCAGCAATCTTTTTTTTTTTTTTTTTTTTTGGGTTTGGTTTTTCAAGACAGGGTTTCTCTGTGTAGCTCT
GGCTGTCCTGGAACTCACTCTGTAGACCAGGCTGGCCTCGAACTCAAAAATCCGCCTGCCTCTGCCTCCCGAGTGCTGGGATTAAAGGTGTGTG
CCACCATGCCTGGCTTGGCATCAGGGACTTTTTTTTTTTAAACATTTATTTATTTTATGTATATGAGTACACTGTAGCTGTCTTCAGACACATC
AGAAGAGGGCATTGGATCCCATTACAGATGGTTGTGAGCCACCATGTGGTTGCTGGGAATTGAACTCAGGACCTCTGGAAAAGCAGTCAGTGCT
TTTAACCACTGAGCCATCTCTTCAGCCCAAAATATTGGTACATCTTTTTTTTTTTTTTTTTTTTGGCTTTTTCAAGTCAGGGTTTTTCTGTATA
GCCCTGGGCTGTCCTGGAACTCACTCTGTGGACCAGGCTGGCCTCGAACTCAGAAATCTGCCTGCCTCTGCCTCCCAAGTGCTGGGATTAAAGG
CATGCGCCACCACTGCCTGGCTTGGCATCAGGGATTTCTTAAACTCACACTGCTGTTAGTTGGAAGGAGGAGCCAGCATCGGCTCAGTGTCTGC
AGCATCAGTGTTCTTTGATGACTGCTCTGTTGCCCTTTGCCCTTCCTAATTCCCTTCCCTTCCTTCCAGGCCACGCCCTTGGGGAGACTTCCTC
AGGCCAAAGCCAACCCACCTCCCACTAAGACACCTCCAGCGTCTGCATCTGGAAAAGCTGTGGCTGCTCCAACCAAGGCAAGTAGGACCAGAAA
CCACAAGATACGTTAGGATAAAATCTCAAAATCTGTAGTCCCACAGTGATCCAGCATGGACCACCCGCTGTCCCACCCCCTGTGATATTACAGC
CACACACTAGTAGAAGGATGACTGCCGCCCTTTCCTCCTCCAATACCATTGCTACCTCTCGATTCAGGGAAAACCACCTGTTCCGAACAGCACC
GTCTCTGCAAGGGGCCAGCGGTCTGTGCCAGCCGCGGGAAAAGCAGGGGCCCCAGCAACCCAAGCCCAGAAGGGTCCCGTGGCTGGCACAGGGG
AGGACTCAGAGAGCAGCAGTAAAGAGGAGTCTGACAGTGAAGAAGAGACGCCAGCCCAGGTACCCACCCAGCTGGACTTGGGGTTAGAGCACCA
TGTGTTTTGGCCCAGGCCTATAGGCACACTTGACATGCCTCTCCCCTTGCTGTTTCTCCAGATAAAACCTGTGGGGAAGACCTCTCAGGTCAGA
GCTGCCTCAGCCCCTGCCAAGGAGTCTCCTAAAAAAGGAGCCCATCCAGGAACCCCCGGCAAGACGGGATCTTCAGCTACCCAGGCCCAGCCAG
GGAAGACAGAGGACTCAGACAGCAGTAGTGAAGAGTCTGACAGTGACACAGAGATGCCATCAGCCCAGGTGAGCTCTGCCAGGCCCGAGAGCCA
CCGTCACCAACTCTGCTCATAAACAGATCGTGCCCCAGAGGGGAGAAAGAACAGATAGAAGTCCTAACTCCAGGATCCCTTGCCTCCAGGTCCT
CCCCACCATGCCCCTGGATGAAATGAGCTGTAAGTCCCAAACCAAGGCCATCCTAGTATAGGACATCCTAGGTACTCCTACAGCTCCGCAGGGG
CCTCTGCAGCACTGGGCACGGCAGTCCCATGCAAGCTGAGTCAGGTTCTGCTGAGGAACCTGGAGCCATCCTGCAAAGACACCCCTGTCAGAGT
CACCAGGAAGCTTGGGCTACGGCGCAAGTCTCTGTCTAAGAAGGGCCCGTCTCGCTCAGTGATGCCTAGGCCCAAGGAGTGGAAAAGAAAGAGA
AGCTGTCTCATACTCAAACCATTGTCCAGTTCTCGGCCCGGTTGGTTGCAGCGAGCAGTGGGGCTTGCTCTGGCTGACAGTCTCTAGTCTGAGC
TCAGGCAGCCTGGTAGAATCGGTACGTGATGGCCTCTGGTGTCGCACCTTAGGGAGGTGGCAGCAAGATCAGAAAGAAAATGGTTGGTCCTTGT
CAGTTTCAGCCGACCGTGGCTTTATAGAAACAAAGGGAATGTCACATATAAGTGGAGATTTTGTAGCTTCTCCTGAGAAACCCAGTCCTACTTG
CCGATTGAGCACAGGCCACCCGAGCAGGGAGACATAGAGCCCTCCCTGGGCCTCCCTTAAGTTGGCATCACCACCAGCTCTTCTTCACTGATTG
ACAAGTTCTGTGGCTTTGGACCACCCGGCCCGTGAAGAATGCTCTTAACAGGGCTTAAGAAGTGTCACTCTTCCGCATTTTCCCCTCACTGGGA
ACCCTCCCGTGTCACGTGCTTACTCAAGTCTTGTCCCCTCCAAAGGATGCTATCTCCCAGCCTGCCAGAGGCAAAGCGTCAGGTCCAGCGTCCC
CAGAGAAGAGCATAGAAGGGTCCTCAGAGAGCAGTGATGAGGATCTGCCCTCCGGCCAGGTAGGCTGTAGCTACTCTAAAGGCTGCCCCTGGCT
GGGCCAGGCTGCCGCTCTCTGTGAGAGGGTGCAGCTTCCCAACAGGGATAGTCTCACCACATGCAGGTAGCACTTCACAGCTCCTAGCTGTCTT
CTGATACACTCCTGGTGTGGTCGGGCCTCAGGCTTCACACCCCTGGCCCCACTCTGCTGCCCAGGAGCCTTTTAGGACTCTGATGTTTTATGTA
AACTGGAGTTGGTCATGGTGCCCAGGCACCTGCCTGAGCACTGTTAGGCGGGCACGTTGTCCTGGCGGCCTGTTCGGGGCCGGGGTTGAGGCCC
CTTATCTTCTCGTGAAGAAGTTTGAGGAACCCAGAACACTACTGCCCAAGCCTCTCTGGTCCCCATGGACAGTTGGGGCTCTAGATCCTCCCTT
GCGACCTTCGTTGGAATCTGTGGGCCCCACCCCAGGTCAGGCCTATGCAATTTATGGCACCTGCTCATTGTCTGCTGGTCCCCACTCCTGCCTG
GCCTTGTTCCCAAGGGTGAGGTCCGGGACACCTTCTCTGATTTCAGGAGCCCAACATAGCCACCAAGATGGTACAAGCTCTGTGTGAACCCAGG
CCTTTGTAGAGGCAGCACTGCCCTCTTTCTGAGGGAGTAGGGTATTTCCATTTGAAGGAGAGAGGGGCCTGTGACTCTGAGGCAGCTCAGAGAA
CATTTGAATCCTGGCAGTCTGCACTTTCTCCTGCCCAGCTGTCCCTTTAGCCCCCTTCCAGGCTTGGCTTTTGCCCTCTGGCTCTGCAGTCTTT
CTGAGGTACCTCCCTCACTCCTAAAGGACAGACAGCCAGTATGGAAGGTTGCCTTGGTTTTTCTCTGTGTGAGTAGACTTGCACAATTAAAATG
AGCATTATGCTAAAATTGAACCTTTAAAGTGGTCTTGCAGATCTGGGGGTCTCTATTTTGAAAAAAAACAAAGCCAATAATTTTCTAGGTTCCC
AGAAGGAATTCAGGGAAACGGGCACAGCCCAGGAAGCTGTGGTTTCCCTCTGGGAGAGTGCTAGCCTACGCGGGATTGCTGTCGCTGTTGGGCA
TTTATGAAGCACTTCTCTATGGAGAGCTGCAGATGGTCCTTCCTAGGGCTCCCCGTCTGGTTGGTTGTTTTTCATTAGCCTGTCCGTGGGAGAA
AAGGCCGGGTAGTCCAGCTCTGGGCCAGCTCTGATCCCAGGTTCTGTCCTGCAGCTCTGTGTAATGCCGTGTGGAGTGTAAGCCTCAAGCTGAG
GCTGGCTGGAACCCGGGGTGGCTACAAGTTAGAAAGAAGTGGGCATGTGAGGTAATGCAGGCACGAGACGGCATGGCTGATCCATTCTGTTGTA
GCCATGTATCGTACTGCGTCACGCACATAGTCATGCGTAACCTCCCACGTAAATTATTATTAAATGTATCAGCGTTTGCCTGCATGTATGTATG
TATGTGCACCACATGCCTGCCCGGTGCCTGTGGAGACGAGAAGAGGGTGTCAGATCCCTCTGGAACTAGAGGTACAGACAGTTGTGAGGTGCTC
TGTGAATGCTAGGAATCAAACCCAGGTCCTCTAACCTCCGAGCCATCTCTCCAGCCCCCTTAAATTGTTTTAAAGGCTACTTCAGCTGCTGTTT
TGCCGAAACAAAGGCATAGGCTATGTCCTTATTGAGTGAGAACAGGGGGCCTGTTAAAAAAAAATCTAAATTTTAGAATTAAGTCAAGGCTTGC
ATGGTTAGGTCTGGCTCCCAGAGCGCTTCCTTCCAGTCCTGTATCTTGAAACCAGTTTGCTGGTCGTCCTGGACGAGGTGCTTGTCCACTCTGA
GAGCCTCAGCCTTAGCTCCGAGTCAGACTGGGGTTTATAAACCTCAGGTCTCCTAGCTGCATCCCCAGAAAGTGTCGTTTCTAAGTCTCATGGG
GAAGGATGAGCCCCCAGGTAATAGTCATCTTCCTCCTCTCTGGGGGGGCGGGGAGGGGTCCTCAGGGGGTCCCAGTGCTGAGGACACACCCTCC
ACTGCACTGCACCTCTAGCCCCACAAAGCTGCTTTGAGGGAAGCTCTCGGTGACTGTCCCCTTCGACAGAGCTCCACCTCCCCTCTCTGCCCCA
GACTAACTGGAATAAGTAGATAGAAGCCCAGCTAAGACAGCGGGCAGTGGGCACCGCCCAAAAGGGCCCCAAATCTGCTTCTGTAAGCATCATT
CCTGCAAATAGGTAGGGTATCCCCCTAATCTGCTCTCAGCACCCCAGGGGGGTGGAGGTGAGATGCTCAGGTGGCTGTGGTGGTAGCGTCCTCT
TCCACCGCTTTGCCTCTCTGATCAAAGCACAGCCCACACCCACTGGGCTTTGTGCTTTCCCCAGGTGACCACACGCAGCACCCACCACAGAATG
AGGAGCAGAGGCGCTACAGACCTAAGCACTGCCCCTGTGCCCAGCCCACCCTCATCAGCCAGGCCGCTGCCTCCTCTATCGCCTGCCCGCTGCC
TCCTCTATCGCCTGCCCATCGCCTGTCGTCCCTGCCTCGAGATGGGGTGGTGTGGGCAGGTGGGGCTGGAGGAGGCTGTGCTGCAGAGCTGCTT
TTAACATTCCAAGACGCTATTGTTTGCCTCAGTGGGTTTGCTGCCTCTCTTGGTCCCCAACAATCTGTTCTTCTGTCTTCTCACATATATATAG
ATCCCCCTCCCTCCACCGCAAGCCTTCTCCCACTTTCCATCCTTCTCCTTCTCATCACGTGGGAAGGGAAGGCTCTGGAAGTGGCAGCTCAGAG
CAGCCGCGGGGGAGGCTGTTTGGCAGGGTAGGGAGGGCCTGAGCGCAGCGGTGCCGCCGCCACACTTGGTGGGTACCATAGTGCTAAAGTTTGT
TGCTTTCTCTGTGTCTCACAGCCCCTGGGGCGGTGGGAGCTTGGTAAAGCACTGCAGGCAGGAGGGTGTCCGTGGATAGAGACCCAGCTACCCC
TCAGGCAGCCCTCTCATTAGCCAGAGCACCAACAGCCTTCAGCCAGCACTGGCGTTTGGAGAGTAGAAAGTGGAATTCTAGTTCCCTACCTACT
GCCTCTGTACCTTCATCGGCTAGGACTCTGAAAGGAAATAGATCTGGAGTCCCCTGGTATGTTGGTGACATATTTGGGAATCTGGTCCACTGCC
AAATTGACCCTCCCAGTACTAGGATCTGCTGCTGCTCTGCCGTCTCAGTGAGATACAGAGCAGACCCACAGGTGAAGTGCATCAGGGCATGTTA
GAACATATCTGTAATCCCAGCATCCAGGAGACAAGGTAGGAATATCAGAGGCCAATGTGGTAGGCTATACAATGAGACCCTGTCTCAGGATTAA
AAAAGTAAAACTGAACCCTACGCATTATCCCAACCTGCAGCTGTCCCGGTCCTGAGCAGGTTGGCTTTGTGCACATTCTACTTGAGTCCTTGAC
TTTGAAGCTGGTCTCAGGCCCGTCACGTCATCCATCAGAATTTCAATATGCAACTCAAAATGATAAGAGTCTTCCTAGTCCTTGTAGACACAAC
CACAAGACAGTATCTCGGCTAGAAAGCTGACCGGCAAGCAGGCCTTGTGCCTGTTTCTCCAAAGTATCTGTCTGATGAGTGGCATTTTTCATGC
TTGTCCTGTTTAAGAGTGAGTCTAATAAATAAGGTTAGTATGTGGAGATCTGTTCCTCTAATTCCAGCAGTGGAGGCAGAGGCAGGTAGATTTC
TTCATCCAAGGATAGACTGGTCTACAGAGCAAAACCTGTCTCAAAGCACATAAGGGCCACATATACACATATATCCTGAGCGCTCTAGTCTGTT
GGCTGGCCTACCCATCCCACCCCCCCTGTTACTCCAGGCAGTTTGTGTACTAAAGAAACTAGTTCAGTATAACTGGGGTCTGCATGCTGAGCCA
CATCTCAGGGAGGATGCTGTCAGGGTCTCTGGGGGAAGGGACAATGAGACTCCAGGGAAATCATACAGTCACACTGTTACAGGAAATGCTTTTT
AGAATTCCCCACTCAGCAGGTGGCCATCTGATGTCACTAGAAGAAAGGCTAAATTAGACATTCCAGCTCTCGCCTTGCTTCCTTCCCATGTGGC
TGTGTGGCCTGGGCGCTCCTCTTCTCCCCTCTGGACTTAAGGCTTTCCTGTTAGGCCAGTGGAGTGGGTGGATACTGCTTAGCTTGGGGTCACT
GTGAAGGTCAGCTGGGCTCTAAGAGCTCTGAAGTGCTGTGCTGTGCTGGGTGTCTGGCTGTTATGGGGTGGGAGTGGGCCTAGAAGACCCCAAC
TCCTTGAAGTTTGCATTACAAAGCCCCTCCCTCACCCATTTCCTCTCCCCTTCCCCTGACCAAGCCTTTAATCAGAGGCGGGATGTTTCTGTTT
TTGGTTTTCAAGGCGATTAAATCCCCTCCAGTTTCTGTCAACCGTAACAGTAGTCCAGCTGTCCCAGCTCCTACCCCAGAAGGAGTCCAGGCTG
TGAACACCACAAAGAAGGCCTCAGGCACCACTGCCCAGAGCTCCTCCTCTGAGAGTGAGGACGGGGACGAGGACTTGATTCCTGCCACACAACC
CTCCACCTATGGTAAGCGCTGCTGCAGTGGGTGCTGTTCGGCCAGGACGGGCCCCAGCCACGCATGCCCTCCCCACTGAGGCCAGAAGGCACTA
ACCAGGTGGCCTAGCAGAGGCATTGGCGAGGACTGGGGACTCAGCCTTAGGCACTGTATTGTTCCCTTAATGAGGAAAGCTCGTCTGCCTCATT
TTGAAGGAATTACATTCGAGGCTTCTGTGTGCTGGCTCCAAGCCTGAAAGCCAGAGGGAGCATTCTGGTGGCTACAGGAACCAGGCTAGTCTAA
ACCAGAGCTCTGCTATAGCCACCTGGCCGTGGGGTACACTATTAGCCTGGCAGTGAGCTTCCATCCCCTTAGCATAGAAAAAGGAGACAGGTAA
CATTTTATACTTATTCTCTTCCCAGAACCTAAGGTGACTGAGTGTCTTTGTATGTGGCGAGGCCCTCTGGTAGCGCAATCATCTTCAGTTATAT
ATCAGGACACTGTGTCGTGCCGGGCGGTGGTGGCGCACGCCTTTAATCCCAGCACCTGGGAGGCAGAGGCAGGCGGATTTCCGAGTTTAAGGCC
AGCCTGATCTACAAAGAGAGTTCCAGGACAGCCAGGGCTATACAGAGAAACCCTGTCTCGAAAAACCAAAAAAAAAAAAAAAAAAAAAAAAAGG
ACACTGTGTCGTTAAGTATGGACAGCAAGATTCCTACTGTCAGCAAAATACTGCTGTTTATTTGGCGAGTACATAGTGGTGACATGAGCTCACA
TTAGGCTGTGGTAAACATCTCAGCACCCAGCAGCTCTCCCACTGGTCCTAACTGAGCAGTTGCTCACTGTGACCTGCACAAGAGAAAAAGATGA
GGGTAAGGCTTGGAGTCACCGCCACAGCAGCCATTTTCAAATGACATGGTGACAGCTGCTGTCTGGAAGGCTTGGGAAGCCAGCGTCCCCTTTA
TGATCCCTAAGCTCAGCATAGGTCCGAGGGAAGAGTGATCTGGTAATGCCTTGCTGGGGAGTCCTACGGCTTTGTATCCTGATGTGCCCCCCCT
TCTCCTCCGTTTCAGCTCTCAGAACCAGTGTGACGACCCGCGCAGCCCTCTCACGAGCAGCTTCCCAACCCAGCAAGAGTGAGCAGTCTAGCCG
GATGCCAAAAGGCAAGAAAGCAAAGGCGGCGGCGTCCGCTCAGGTACTGTCGGGTAAGAGAGGGCTGTGGTGGGGTCACTCTCTGCTGCCTTGC
CATGTGTCCTCAGGCCACACGCCAGTCACTAGCTTGCTCCCAGCCCCCTGGCACTGTCATACAGGCCAGGAGCCAAGCTCCCAAATCCCATGGT
GTTGTCCGGAAGAGCGGGCTGGGAGCCCGAAGGCAAGCGGAAGCCCCTGTCCTCCCAGCTTGCCTGAGTGTGGATTCGTTTTCTGGATTTGAGA
CGCTGAAACCGACTCTTGAGTTGCTTGCCTCTCTTAGTATTCGTAATGGAAGTGGCCTTACTGTTTCCTCCAGCCTTCACAGTCCAGTGCATTA
TTAATATCTAGGGCAAGATTAGTGGACAATATCTAAATAAAACAGGGCTCATCCCACCAGGGTTGGTCCAGGCCAGAACCTGGGTCCTCCAGCT
CCAAGTTCTTTGCTCTGGAGGCCTAATGGGGTTTGTTAACGTGAGGGAAGAGTCTATCGCACACCCAACACCCTAAGGGGCAGGAGAAGAAGGC
TGTACTGAAGCCCCTCCCAGCTTCGGGGTGCTACTGTGCCACCCTGTTACCCGGTGCATCACAAACTAGCCCAAAAAGAGCCTCTGGCGGCCGG
GTGATCCAGTGGGTAGTGAGATGGCCTGAGCATCTGCCCTGCTGGGCACCGAGGCTGCAGCAGGCACGAGACCTTCACGGAGTCCTAAACCAGC
AGAGACACAGCCATTACACAGAAAAAGCAGCATAAATTAGTTGTCACCAACTGCTAGGTGTTGCAAAGGGAACAGCACAGAACAGAGAATAATG
GGTGACCTGCTTTAGAAAAGGCGGGCAGGAAGGCTTCTCTGGGAACATGAGCAGAGCCTGCCCTGCAGGAAGGGAACCCAGAGGTACTAGAGCC
CTAGGTGATTGACATAGGGCGGGTAAGATAAAGTAGCAAGGGGGTCAGGCCATACCTGGACTGCTTTGTGTCTGCAGCTCCAGGAGGCTTAGCA
AAGGCTCATCTATTGTCTGTGTGCTCTTCCTGACTTCTAGCAGGATGGGAAGCAGGGGTGCCCTGAGGGACCACCTAGCCAGGGACACATCCCA
GAGGAGACACAGGCGATGGCCACTGGAGTTGGGGCTGCGCTGTCCTCCCCTGGGCCCAGATTAGAGAACAAAGCCATAGCTCACCACCTGGTAC
CCATCTTTCCAGCTGCGATGGCTCCTCTGTTCTCCACCTTGGCCTCAGGGCTTTTCTTGCTTGAACATCCCTGCCTCTTTACCCATGCCAGTCA
TTTCTGGCTCAGGGTAGCACAGATGCTGTTCTGAGAATCTATGGGTCTCAGGGCCTCCTCGTACCATCAGTGGATAACAGAGGGACCCTGGGTC
TCTCCCACTGGTGTTGAAAGCTCATTCTTAAAAAGCCAGTAAGCCGGGGGCTGGAGAGATGGCTCAGTGGTTAAGAGCACCGCCTGCTCTTCCA
GAGGTAATGAATTCAAATCCCAGCAAATACATGGTGGCCCACAACCTTCTGAAATGAGATATGATACCCTCTTCCAGTGTGTCTGAAGATAGCT
ACGGTGTACTTATATATAATAAATAAATCTTTTTTTTTTTTAAGCCAGTAAGCATGGGGGAGGGGCACAGCTTACTTACTGTGCAAGCATGAGG
CCCTGAGCTCATTCTCCTGTACCCACAGGGGAAAGCCAGGCATGGTAGTGGGCGTTTGTAATCCCAATACTGGGGCACGTTAACCAGCCAGCCT
AGCCTAATTGGTGAGCCCCAAGTCACATTAAAAGACAGAAGGCTTCATATGACTGGGTTCTCTTCTGCTCCACCTCAGTGAAAGGCCAGCATGG
TTGTGTGTGTAGTCAGCAGCCCCTCTTTCACCTCCCTGGGCCTCAGTGTGCTAGTGCCAGGTAAGGAGCCACAGTGCCCACCAGAGGGCTGTGG
GAAAGTCATAGAAAGCCATGCGTAGTGCTTCCCACGTGGAGCCTGGCCTGCCGTGTCCTTCTCACTGTAACTTAACCTTGTGTTCCTACTTTAC
CAAACTAGACCAGCAGTGCCGTGGAAACACTCCCCATGATGCCTCCCCAGAGCGCACCCATCCAGCCCAAAGCCACCAACAAGCTCGGGAAATC
CAAGCTCCCTGAGAAGCAGCAGCTTGCCCCAGGCTACCCCAAGGCCCCCAGGAGCTCGGAGGACAGCAGTGACACTTCTTCCGAGGACGAGGAG
GATGCCAAGAGACCCCAGATGCCCAAGTCGGCCCACAGGCTGGGTGAGGATTCCCAGAGGGAGGGTGGGAGATGAGGCACAGCCTGGTAGAACT
GTAGGCTCTGGCCTTCTCAGCCCACCCTGGCCTTGTCTCATCCCCTGATAATACCTTTACAACTTCAAAGACCCAACTGCTGTTCGACAGCTGG
CAGCTTCTTGGTATTAAGCTCCGGTTATTGAAGGCCCCAAGTCCCAGGACTCTGCTTCCCTTGGGGGGAGGGGGGGCAGAAAAGTGGGAGCCTC
AGGAACCAGAGGGGAAATGATGCCATTCGTGTGTCGGGCTAGGTGCTCCAGACTAGCATGGGTGGACAGCAGGAGGCTTCGTGCAGGAGGATGA
GGAACAGAGAAAGACCCAGGCCTCGGAGGCCTCCTGGGGAGCTCTGCCTGGCCTGAATAACAGGGCCGCCAGGGACTCACACTCAACGGGAAAC
CTCTGCTTCCACACTAGATCCAGACCCTTCCCAGAAGGAAACTGTGGTAGAGGAGACCCCTACAGAATCCAGCGAAGATGAGATGGTGGCCCCC
TCACAGGTAACTGCCAGGCAGGGCTGGAACTTGGAAGCTGCCTTTAGACTTCAGCCACCCCCTGGATGAGTGTCTAGATCCCCATTCCCAGGGC
CCTGGGTAAGGTCCTGTCAGTGGGGTGCCTCTAATCAATCGGATAGAACCTCACCTCATGCTCTGGGGCCCAGGGAAGAGATTCAGATGGATTC
TGAGACATGACCAGAGAAGACGACTTCTTAGGTCTCTCTTAAGGCTCTTCCTATACAGGACAGACTCCAGTGACTTTCTCACGTGTGGGGAGTG
GGTGCGTGTTGGGGTCAGTCCTCTCCTTCCACTTTCACGAAGGTTACTGGATTGTACTCAAGCTGTCAGGCTCGACTCGTGTCAGTGCCTCTAC
CCTCTGAGCCATCTTGCCAGCCCAAAAAACGTTAACCATCTTCTGTGATGGTACTAGTCCATTGGCAATAAGAAAGTCCCCTTTTAATGTGCCT
TACATATGCGTTAATTCACATAGCTTAGAAGGCACAAGCTGAAGGCCTATGTCCCCCGTGGGCTCAGTGCCGCCCGCTCATCCTTGGCTGGGTT
GGCCTGATCCGGGGTCACCGACTGATTCCCTTGAGCCTTGCCTCTGGGATACGGCTAGGGGCTCGATGCGTCTTTCACACTGGACTTCTTCAGT
CTCTCCTCTCAGGTTACATGACTCCCGGCTTGACTGTGGCTAATTCCCAGGCTTCAAAAGCTACTCCTAGGCCAGACTCCAACTCCTTAGCTTC
TTCTGCCCCGGCCACCAAAGACAACCCGGATGGCAAGCAGAAGTCAAAATCCCAACACGCAGCAGACACCGCACTCCCTAAAACCGGTGAGTGG
GGACAGCAAGACATGGAGAAGCAGGGCTGCTGGTCCCTGCTTGTTATGGGCAGCATTTGTCTGTGTCCCTTCCAAGAGTCTCATATTCGAGCCT
GTGGGGTCCTCTGGGAACTAGAGGTACCCTTAAATGCAGCTGGCGCCCAGGACCAGGTTGCCAGCTCTGGGCAGTCTGGGAACAGATATAGCCA
GCCATTTGAAGCTATTTCTTTAAAGCTTGTGGAGGAGGCCTTGGCTCTCCGTCCGTCCCTACTGACTTCTCAGTCAGTGGACAAGGGTCCTGGG
TATAGGGACCCAGAATGACTCAGACTAGGCCAGCAGCCTTGTTTAGAGAATTCAATACCTAGAGGAGACCCTGGATAAGGGACAGGCCTCCGGG
GGCTGCTCTGGCTCACACTGCTGTAGCCTTGACAAGCTCCTGACCCTGTGTCTTAATCACACAAGGTAAGAGCAATGCCATCGTGCAGGGTCAA
AAGCAGAGCATAAAGAAAACTGTCACATGACATTTGGAGGGACCCAGGGGAAGGGACTGTTGGGAGTGCCCACATCCCTGACTTATATAACATG
AAGCATCCCGTCCCACTCCTGTCCGCTTCTCCTTAACCGCACTCACTGCTAGGGTTCAGCCCCTGTGCCCAGCATGTAGGCATTCCTTGCTGCA
CAGGTGACCTGGGAGGCCAGCGGGGGTTCTGGGCCCAGGCTCTGGGGGCTGGTAACTTCAGCTTCCCAGCGCTGGGTCAGAGGCGGAGCTGTTG
CTCACTTTCTTCGCCAGTGTTCTTGTCTATGTGAGAGGCGGGTGCGAGCGACAAGGAGACATTTTCGAGGGGGCTGTGGAAGGTTTTTTTTTTT
TTTTTTACACGTGTAGTCAGCACACAATTATCAAAGGCAAGCAGAGGCGTAGAAAATTCTAAACAAAGGCTGCACCAGAAGCCACTCTGGCAGG
CAGTAAACCTGCTTAGTCAGCCACTGCATGCATACACAGCATACTCAGGAGTGCCCCCCAATCTGCCTGTCGCCCCAGCCTTGATGGGCTAAGG
ACCCATGGTGTGTCTACAGACGGGAGCTGAGCATTGGTCTGTAGGACCCCCTCGTGTGTTATAACCCACAGTTCATGGCAGAAGACATCATTGT
ATGTCTAGCTTCTTCCTCTAAGGTGGGAAACAGCCTTGAGGGACTGTGACGCCTTGAGGGACTGTGACCTGGCCAAAGCCCTACACTAAGTTGA
TAAAGGCAGTCCTAGAACCAGTGTCACCCATGTCCCACCTACTGTGCCCTCCGTCTCATTAGCAGACTTCAGGTCCTCTCCTTAGCATTCTGGG
ATCTCTGTCGATCCACAAACATAGCTAAGCATAGGAGACAGATAGAGCAGAGTGACCCCTGGATCTCAGTTTCCCATTTATGCAGTTGTAAAGT
ACACCCCCTCAGTTGTCATGCATTGTGCAAGATCTCACCCTCAGCTTTCCAGACTTGTGACCTCCAAGACCTGTGATCTACAGGTAGGAAAGAG
GCCTCCTCGGGCTCCACGCCTCAGAAGCCCAAGAAGCTCAAGAAGAGCACCTCAAGCTCGCCGGCCCCCACACAGACACTGCCGAACAGCATCA
CCCAGCGCCTCCTGGAGCAGGCCTGGCCCCTGAGCGAGGCGCAGGTGCAGGCCTCTGTGGTGAAGGTCCTGACAGAGCTTCTGGAGCAGGAACG
GCTGAAGGCCACAGAGGCCATCAAGGAGAGTGGGAAGAAGAGCCAGAAGCGGAAGTTATCAGGGGACCTAGAAGCTGGGGCCCCAAAGAACAAG
AAGAAGAAGGAGCAGCCAGTGCCCAGGGCAAGCGCTGTTTCCCCAGAAAAGGCTCCCATGACTTCCAAGGCAAAATCAAAGCTTGACAAAGGGA
GTGCTGGTGGCAAGGGGAAGGGGTCTCCTGGCCCCCAAGGAGCCAAGGAGAAGCCCGACGGCGAGTTGCTGGGGATAAAGCTTGAGAGTGGCGA
GCAGAGCGACCCGAAGAGCAAGTCGAAAAAGAAGAAATCCCTCAAGAGTAAGTGACTGCTTTCCAGTACGCGCTGCCGGTGCAACCTGGGTGGC
AGGGCCCTGCCCCGGCTCTTTATCTCATTCTTTCCACAGAGAACAAGAGCCTGGGAAGTGGTTTGTCCAGAGTTTAACAAGTGTCTGGTGGCAA
AGGCAAAAGGAGACTGTAGAGCTGGCCTGCGTCCCCTGCCCCCACGGCCCCTTACAATGCCCAGTATTTAGCCTCAGGGTTTGGAAAACAAACT
CAACCCCGGCTTCACAACAGGGAGCTCTCTGCTCCAGCAGAGTCCACGGCCAGTCCCTGGGGTGTGGACTGTCCCCTGGGTTCTCATCCAGAGC
CCATCTTTACTCTAGAATTCAGTTCTTTGGTGAAAACAGCCATTCGTTGCCCTCCATGCGGGAACCTGCACCCAGCAGGACTTCTGAGTCACTC
ACCAGCCCTGGGCCTCGGGCAGTGTTGTTTTCCTTTTCTAAGGGGAAAGCAGGCTCTGAGAAGGAAAGACCACTGGCGCTAGGCAGTCCCTGCT
TTAGTTTTGTCTTAAGACAGGGCCTCACAATGTAGCCTTGACCGGCCTGGAACTTTCTGTGTAGAACAGGCTGTTCTCAAACTTACAGCAATCC
ACCTGCCTCTGCCTCCTGGGTGCTGGGATTAAAAGGGTACTCTACCACGCCCAGCATGGAAGTCCTGAGCACTGCCATTCTGTGGCAGGAGATG
TGTCACCTGATCTCTCCCAGGTTCTTGTCCCTTCCCTCACACTCAAGATTAGATAGATAGACTTGCATAGTTCTGAGGCCAGATTGTGTAGTCC
ACAGTCAGTAAGAGTGGAGCCTGGCACATGGATGGTATCCTGGCCCTATCAGGGCCTGATGTTAAGAGCCGCGAGCTGCCGTGGGCCTCCTGAC
GCTGACCCATCAGTGTCAAAATCTATTGCTCAGGAAGCTCGCTTTCTTCCCACAGAAAAAAAAGACAAGGAGAAAAAGGAAAAGAAGAAAGGAA
AAAAGTCCCTGGCCAAAGACTCTGCCTCGCCGATCCAGAAGAAGAAAAAGAAGAAGGTAGATTGAGTTCCTGAGGGGTCTCAGGGCTAGGAATT
CAAACCCTTCCCCTTCTAACACCTAGACCAGGCCCATGCCACCTTTCCAGTCCCTTAATCTAATGGACCTTGTGAGGAGGAGCAGGGGAGGGGT
GATGGGAGAGACCTCCACAGGCGGGACAATGGTCAGCTTTCAGGTTGGCTGGCCAGCGGGGAGCCCACTGAAGAAACAGCCAGCCAGCCGCTTC
CTGGGATCAGAAGGCAGTTCTGCCCAGGGCTGGTATCAGTGGGTCTCGATCCTTACACCTATGGGGGGCCGTGGATCCTTCGAAGGGAGTTCTG
TCCTGGACCTGTGGGACAGCGGTGGTGAGGAGAAGCCACAGAACTGATAGCTTGCTTCNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNAGAGG
GAGGGAGGGAGGGAGGGAGAGAGAGAGAGAGAGAGAGAGAGAGAGAGAGAGAGAGAGAGAGAGAGAGAGAGACTTCTTTCCTCAGGTTGGTTGG
ACAGCAGCTGCTGCAGCATGCAGAGGGTCCACCCTGAGGGAGTCTGGTGCCTAAGCTAGCTGCCGCCCATCCCTCCCTTGGTTTTGTTCCCTTT
TGTGGAGATAAAGGTCGGCAAAGCTGACCAAAGCCTGGTTTGTTTTCTTCTCCTAGAATCTTGGCAGAGTGTGACCATCCATCCCCAGGCCTCT
GACCTCTGGCCAGCGTGAATTGGGCCTGGACTTTTTAACTTTCCCTCCCTCAGCAGAAGATGATGGCTGGCGCGTGCTGGCCAAACCAGTGAAC
CCGCAGGGAAGCTGCAGTACCAGGAGGAAGCGAGGTGGGACACACACCCCCTCTGAGTGCTACGTGACATGTGTATAGTATATCTATTTTTTTA
AGCGACTTGCCTCTCTCCCTTCAAATCCACCGTGCCCAGAGGCCTCCGGACCTCCCTCCATGTTATCTGCAGAGCCAGCAAGGTCCCGCGTGTG
GTTCATCCCACACCCTGGACCCCTGGCGCAGCCTGAAGCCTTGCCCCATCAATTTTTCCAGCTATTTTGGTTTTGTTTTTTAAAGAACTCAGAA
TAAAAGACTTTAAATATAAAGGTTTGCCTTCCCCATGCATCTGGGACTCGGACTGTTACTGGCTACTTAGGACAGACAGTGTAGTGACCCTGGA
CATGCACAGGTCAGTACTCAGGGCTCCTTCCTGACTTCAAGCCAAGGCCCTAGGTACTGTGAGTAGGACAGTTTGCTAAAGAGGAAGGCTCAGT
GAAGCTCCCCAGGGAGGGAGTTGATGCAGGGACGGCTCCCCGTAGGAAGTGGCCTATAAGCTTAAAAGGAGCTGAAGGTGGGTCAGACGCCAGG
TAGATGGCTCAAGTGACCAGGGGGCCTTTGGAAACAAGTTACGATGTGGAGGGAGGCAGTGGCGAACAGTGGTGGCTGGCATGAGAAGCGGGGA
AGACCCAGTGAGCTAGGCCTTGAGTCACCCTCTGTCTGGGGAACAGTTCTGTGCTGGTGACAGTAAGAAGTGGGCAGAGCTGAAGTATGTTGGA
ATAATCACTGAACATTCACCTCAACAAAACAAAAAACCAGTACAGAGAAGGAGCAGGTGCAAGTCTCGGGGACCCTGTGCTCAGAGCCTTGGGA
CGGGCACGCCATACCCAACAGTCAGCCATTCCATCCATTCTCAAGTGTGGATCAGAGTGTGGGATGTAGGTCTAGATCAGAATACAGAGAGCCT
CTCCTTGGCTCGGCTGACTCGCAGAAGTATTTAAACCTACAAAATGGAACAGCGTCCTTAACGTTCATCTCGGAGTCCCTGGTGGTGAGGGATC
CGCTCCCTTTTCCTGTGACAGTGGGCTAAAGCTGACAAGACACAGGTCCTGCCCAGACAGACAGCCACTGGCAGCCCCCACCTGCATCCATTCC
TGACACTAGGTCCCTGTGTCCACTGCCATCTGTCATCACACTTACGCTGTCCTTAGTCCAGAAATGGAATTGGGGACGGGAGGAATGGAAGCTA
GGACGTGATGAGATGTGATTTTAAAACCGAGCAGGAGTCTGTTTCTTTCTGACTGTTAAGTAGACACCACGTGTGTCTGGGACCCATCTCCCCA
GTCCATGTACGTAGAGGATTAGATTTAAGGTTGTCCTCGGCCTTGGGGTAAACTAACTGGATCCCAACTGAAGAGGCGGAAGCAAGAGTGTTCT
ACTTTCTTCCTTTATTGTCACTTGAACCATGATCCTACAGGCTGCTGACAAGCTTGGCTGAGCAAGGAACCTGAAAGGGGGTGGGGCTTGAGGG
GGGGTGGCAGGATGTGAGGAGGGGTTACTGGGAGGAGGGGCTTGGGTTCTTCTTCCTTGAGGAGAAGGCAGCAAATGTGTCCAGCCTAGGTTAA
GGGTAGGGCATCCCTTGTCCCTGTCTGCGGGCCTTTATCTCAGAGCCCCACATTCAAGACTACTAATGGGTCAGAAATGGGGGTTCCCTTTAGA
TAGCGGTAGCCAGCAAGGCCCTCCAGCGTCCTCCTTCTGTTCTGACTTCATTTGCCGTGTCCTGGGACGATGAAACAGACACCAGTCTCAAGCC
CCAGGGTGCAGGGAGACGGGAAGTGGGGTGTGACATCAGGGAACATAAGAAGGGCTGAGGCACAGGAGAGCAGGGGGCGCTGCTGCTGTGCAGA
GCTGGCCTCTGTCTTCACAGGGTGACTGGGAAGAGAAGCACAGGTTGATACAGCTGGGCCACCAGTTTGTTGTCCCTGCCTCCTCTGCCCAACA
TCTTGGAACTGCAGATCAGTCTGTCCCCCTGCTAAGCTCAGGTAGTACTGTGCTCCCAGCCACCATCCCATTCTAGAAAAACCATCCCAGTGAT
AGTGATGAATGTCCCAGTGTCACCAGAGTCTGCATGTGGGTGGCCCCCTCTGTGATCCCCCCCCTTACCTTGACCCAGTTCCTGCCTGGTCACT
CCCAGGCCAGAAGATAGGTCTTCCATGTCCAGTGGCTCTGCAAGGGAAGCATCAGGCTCACTGTTATTTTTACCCCAACACACACACACACACA
CACACANNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNACATTAGCCACACCAAGCCTGGCCCCTGGTCCCCCATGTTGGAGAGCAGCGGTGAGGGGCACCCAGCTATG
GAACTCCAGTCAGGGTCAGGAGCTGAACCAGCACCTCCAGTTTTTTCCCTCTGTCTGGGTCCTCTGCCTGTCCTCACACAACCAGTGTTCCCTG
CTGTAGGCCAAGAGCCATGTGCCATGCACTGTGCCTGACACGAAGCAAGCATGTGCAATATTAATTTGGGAAGGAGAACATGGTATTGTGGCCA
AGTCAATCCATTGGACCTTAATTTGATTTTTTTAAAAGATTTATTTATTTATTTCATGTATGCGAGCACACTGTCTCTGTCTTCAGACACACCA
GAAGAGGACATCGGATCCCCATTACAGATGGTTGCGAGCCACCATGAGGTGGTTGGGAATTGAACTCAGGACCTCTGGAAGAGTAGGTGGTGCT
CTTAACCTCTGAGCCATCTCTCCAGTCTCAATGGACCATAATTTTTAACTCCTCAAAAGTGAGGTGGCTGGAACCTGGCCTAGTGATGCACGCC
TTTAATCCTAGCACTGGGAGCCAGAGGCAGGCAAATTGTGAGTTCAAAGCCAACCTGTTCTACATAGTCGGTTCCAGGACTACATAGACTCTTA
TCTCAAGGTGGGTAGGGGATGGACTGTACGCAACAGTTAAGGACCACCTTGTATTCCTTGGACTAAGCATGTGTTGAGTTTATAGTGAGCCTAA
CCCATGAGTGAGGGCCGGGGTCCCACGTGACCCAGGAGACTACCCGCTGGCTCTGATACTCACCGTGAGCAGATGCATCACATGGTCCTGGGTC
ATGTTGCCGTACTTGGTAACGTTCTTCACAGGCTGTGGGGAAGGGAACAGGGTTACCAGCATTGCAGGGATCAGCAGCTATAGTCTAGTACGAG
TCAGGTGCTTGCACTCAACAGCTCAAATCTGCCACCTCCTAACATGGAAGTGCCAGCCCCTCACGCCCTTCCTGCCTCTATGCCCTCTCTGGTC
AGGCGGGCAGAAGCTTGTCAGAGCCTTCCCAATAGCAGAGTTTGCTTCCTCCTCCCAAGCCCTCCCATATGTTCCTTCCTGCTTCACTTCAAAT
TCATGGCCTCTTTTTTCATTCATTGTCATTGCTTGCATATATTATATGTCTATATATATACACCACTAGTCCTAATTAAACCTGCCCCGTCTGT
ATCATGCTAACTTATGTATGTTTTCAGGACTGACCATTTGGCACCGTTCGATTGATGTGCTCTGCCCTGGATAAGGTACCAATAGTAATCCTAT
GAGTAACAGCATTTTTGCAGCCAAAGACTTGGAGGAACTGAGGGACTATAGGATGCTGAGGTGAAGAAGCACTTTGCAAGCTGTGGGACTTGCC
CTTTCCCCACCAGGGGAGCCCTAAGAGTTCTGGCCTCCTCAGTCACCTTGGGCAAGCCTCTCTGAGGTGTGCCCTGCTCCTCGCCAGTAGTTAG
TGCCCAGATTCCTTGACAACTGTTATCCTTCGGCCCCTCCCCTTGGCAAACACCAGCTACCATTTCCTTCCTGCCGCCTTCCTTACCCCAAGGA
GCATGTTATCCATGGACATTGGACGCATCAGCAAGGGAGTAGCCATCCGCATCTGGCTCACAGGTTTGGCAGCTGTGGGGCAGGGAGGACAGTA
TGAGTCCAGGTCCAAGGGAGTAAGGAACCCAGGGGTTAAGATTTGAAACCCGGTGCGGGGGAGGAAGGGAGATCGGGAGGGGCTAAATGGAGAA
ATGATGTCTGTGAGGTGAGGGACAGGTGGAGCACGCACATTTCGGAAGCTTCATGCGAAGGCTCTCCAGTTGCAGGTTCTGGGAGGTGATGGTC
AGCTTGTCTAGGCGGCCCTGTTGCTGGTACAGGAAGTAAGCAGTGGTGGCCTGCCCAGCCAAGAGCAGAGCCACCAGGACAGAAACACCGGTGT
ACAGAGCTCCACGGCTGCACCTGTTGGGATGGAGTAAGAGCATGATCCAGAGCTGGACGCTGACTGGAAGTGGGGACGTAAGCCCGGACCCACG
CCCTCTCAGACTCAAACCCTCCCAATAGTGAGGACATCTGCTCCTGCCGCCCCCCAGTGTCCTTCCCAGGAAGATCCACCTGGGGCCCAAGCTC
AATGGGAAGGATGAGCCACGTTCCTTGCCTCCTCTGGGCTTTGGTGTCTCCTCTGTGTAGTGACAGGGTAAAGTCCTTCCTCCTTCCGAGTCCC
CTCTACCCTAGAGATCTGAGGAGCTAAGCCCCCCTCGGAAGTGGGGAGGGGGATTTGATCTTGTTTTTGCTGGCAGAAAAAGATAGGTGAGTTT
ACTATGTAAGAGGAAGAAGGGTTTTCATCTACTGTGCCGTCCCCAATCCCCACGGTGACGTCAGGTTTAAAGAGAAAAGGGAAGGAAGTGCGGT
AGGGGACAGCAAAAGCCGGATTTATACCTGAAAAGGAGAAGTGGGGCCTAGAGTTTTTACATGTGGAGCGGGAGGAACATTGTCAGGCTGAAGC
AGTGCCAGGCACGTGTGCGTGCACACACACACACACACACATCTCCAAGACAACCCCCTCACCCCCACCCCTGCTCCAGAGTCCGTCTGTCCCA
GAGTCCCTGGAAATTTGAATAGATCTTCTTCCCCAGTCTCCCAGCTCTCTCATATGTCATGTGAGCAACCCTCAAACCCACAGTGTTAAAATTT
GGCTGTCCTCCAACAAGTGTGTGTGTGCGTGTATACTCATGTGTGCTGTGCATGTGTATGTGNNNNNNNNNNNNNNNNNNNNTATGGCCTAGAA
GCATCTTGTAGTGATCTCCAACTGGGAAAGTTCAAGAGCAACACAGGTACCTAGGCTTCATGCTCTTGGACAGCCAGACTCTCAGACTTCAGTG
ACTCCTTACAGTTTGGGGGCTTTTTTGGGCTGTGTTGGGGTGCATGCACATGTTACAATATGGAACACAAGGACCCTGACTGGGCACCCTGAGA
TTTCCTGTCCAGTCTTCCCCACTCACCCTTGGGTAGGGAGAACTATCAGTAATGTTATTTGCACACATCCCCTTTAAGATTCAAGTGTTGGGGT
TGGGGATTTAGCTCAGTGGTAGAGTGCTTGCCTAGCAAGCGCAAGGCCCTGGGTTCGGTCCTCAGCTCTGGAAAAAAAAAAAAAAAAAAAAAAA
AAAGACAAAATAACAAAAAGACCAAAAGAAAAACAAACAAAAAAAAACAAGGTAAAAGATTCAAGTGGATGGATGGAGTTAGGCACCGTTGTAG
GAAGGCTTAACAGGGGAGTGAGTCTCCTGCCTTTTCACTGCCTTTCTATTTACAACACAGCAAGAAACTCATCATTAGGGGCTGGAGAGATGGC
TCAGCGGTTAAAAGCGCTGACTGTTCTTCCAAAGGTCCTGAGTTCAAATCCCAGCAACCACATGGTGGCTCACAACCATCCATAACGAAATCTG
ATGCCCTCTTCTGGAGTGTCTGAAGACAGCTACAGTGTACTTACATGTAAAAATAAATAAATAAATATTTTTTTAAAAAAAAGAAACTCATCAT
TAGATGCCAGCGCCTTGATCTTGGGATTCTCAGCCTTCGTAACTATGGGAAATAAATCTCCAGATTTTTATTATTATTATTATTATTATTATTA
TTATTATTTATATAAACGACCCAGTCTGTGGCTTTCTATTATAGTAACACATACTCAGACTAAGTAAGACGAACTACATTGGAAATCCTCGTGA
TAAGTTTGCATCTTGCCTACTCCAGCAACCAGGAAGGGTAATTTAGGAAACTCCAGAAAACCTGTCTGCCTGGTAACTTATGACATCACAGACA
CAGCTAATTCTCATTGGCTTGCCAACCCTCTGTATTTCCTAAGCCAAGGGCTGGAGAAAGGAGATCAGACAGGAGGGAGAATAGGTTTAAATTT
CTTGGCCTTCTGAAGGAAGATGGAACCTGGGCTCAAGGTCTTCTGCTTCATCTACTTTGGTGACTCATGGAACCCCCGAACCCCTCTACCACCC
CATATTCAGATCTAGTCCCAGCTCTACCTGCTACCACATGAGCTTGGGGTAGCCACGTCCCTCTCCTTTCGAACTTCAACCTTTCAGAAGGCCA
GTGAACACAGGTTTGTATTGAAGATGTGAACAGTAGCCATAACTGGCATCCTGGCTTGCCTAACTCTGGGGCCAGGAACAAGACACTTCTGCGT
GTGGACCAGGTTAGGGCTTGGAATGAGCGGTTGTTAAATAACGAAGAGATGAAACATAAATAACGAAGAGATGAAACACTTGGCCCCACACCGG
ACACACATTTCCTCTACCAGTAATACTGCTCTCAGGAAGGTCACTATCCATAACAATGCTGCCCTCCAGGAGGCTGGATGTCAAGCCCAGCATC
ATCTCTCCTTCCACTTTTATTCAATTTAATATTATTTTGTTTTTCTGAGATGGTCTCATGTAGATGAGTCTGGCCTTGAACCCCTGATCCTCCT
GCCTCCA
MOUSE SEQUENCE-mRNA
TAGCGGGGGCGAGGGGGCGTGCACGTGAATCCCCTGGCCAGATCTCTGGCATGGCCGAGGCCAGGAAGCGGCGGGAGCTGCTTCCCCTCATTTA
CCATCATCTGTTGCAAGCAGGCTACGTCCGCGCGGCGCGGGAAGTAAAGGAGCAGAGCGGCCAGAAGAGTTTCCTGACTCAGCCCGTCACCCTT
CTGGACATCTATACACACTGGCAACAGACCTCAGAGCTTGGCCAGAAGCAGAAGGCAGAGGATGATGAGACCCTTCAGGCTAAGAAGTCTCGAG
TGTCGGATCCTGTTAGCAGCTCAGAGAGCTCGGATCAGGAGAAGGAAGAGGAGGCAGCAACCGAAAGGGCCAAAGCCACCCCAAGACCGACACC
TGTCAATTCTGCAACCGCAGCTTTGCCATCAAAAGTAAAAGAAAAGGGAAAGACCAAGACAGCCAACAAGACGGTGAACTCTGTGTCGCACCCT
GGGTCCGGAAAGACGGTGGTCCACCTGCTCTCTGGGAAGTCACCCAAGAAGTCAGCAGAGCCCTTGGCAAACACTGTCTTGGCCTCAGAAACTG
AGGAGGAGGGCAATGCCCAAGCCCTCGGACCCACTGCCAAGTCTGGAACGGTGTCAGCGGGCCAAGGCAGCAGTTCCAGTGAAGATTCCTCCAT
CTCAAGCGATGAGACAGATGTCGAGGTGAAATCTCCAGCAAAAGCAGCCCAGGCCAAAGCTTCAGCAGCCCCTGCCAAGGATCCTCCAGCAAGA
ACAGCCCCAGGCCCTACCAAGTTAGGGAATGTGGCGCCCACACCTGCTAAACCAGCCAGGGCGGCAGCGGCAGCTGCTGCTGCTGCTGTGGCTG
CTGCTGCTGCTGCAGCAGCAGAAGAGTCTGAGAGCAGTGAGGAGGACTCAGACAGTCAGGACGAGGCCCCTGCTGGTCTGCCCAGCCAGGTAAA
AGCCTCTGGAAAAGGTCCCCATGTCAGAGCCGACTCGGTGTCTGCCAAGGGGATCTCTGGGAAAGGGCCCATCTTAGCAACCCCAGGGAAGACT
GGGCCTGCAGCCACCCAGGCCAAGGCAGAAAGGCCAGAGAAGGACTCGGAAACCAGCAGTGAGGACGATTCTGATAGTGAGGATGAAATGCCAG
TCACTGTGAATACTCCTCAGGCAAGGACTTCTGGGAAGAGCCCTCGGGCCAGAGGTACCTCAGCCCCCGCCAAGGAGTCATCCCAGAAAGGGGC
TCCTGCAGTCACCCCTGGAAAGGCAAGGCCTGTGGCAGCCCAGGCAGGGAAACCAGAAGCCAAGAGCAGTGAGGAGTCAGAGAGTGACAGTGGG
GAGACACCAGCTGCTGCGACTCTGACCACGAGTCCTGCCAAGGTGAAACCTTTGGGGAAGAGCTCCCAGGTCAGACCTGTTTCCACCGTCACCC
CGGGGTCATCGGGAAAAGGTGCCAACCTGCCCTGCCCTGGGAAGGTGGGGTCAGCAGCTCTCAGGGTCCAAATGGTAAAGAAAGAAGATGTCTC
GGAGAGCAGCAGTGCAGAGCTGGACAGTGACGGGCCTGGGAGCCCAGCCAAGGCAAAGGCCTCCCTTGCGCTCCCTCAGAAGGTGAGGCCTGTG
GCCACCCAGGTCAAGACTGACAGGGGCAAAGGCCACTCAGGGAGCAGTGAGGAGTCATCTGACAGCGAAGAGGAGGCAGCACCAGCAGCCTCTG
CTGCTCAGGCTAAGCCAGCTCTGGAAAAGCAGATGAAAGCTTCCTCTAGGAAAGGCACGCCTGCATCCGCAACAGGAGCGAGCACCTCGTCCCA
TTGTAAGGCAGGAGCGGTGACCTCTTCAGCCAGCCTGTCATCCCCAGCTCTGGCCAAGGGCACCCAGAGGTCAGATGTGGACTCTTCCAGTGAG
TCTGAGTCAGAAGGAGCTGCTCCCAGCACCCCCAGGGTACAGGGGAAGTCTGGGGGCAAGGGCCTCCAAGGGAAAGCTGCCTTGGGGCAAGGGG
TGGCCCCAGTGCACACTCAGAAGACAGGGCCTTCGGTCAAAGCTATGGCTCAGGAAGACTCAGAGAGCCTCGAGGAGGACTCCAGCAGTGAGGA
AGAGGATGAGACCCCAGCACAGGCCACGCCCTTGGGGAGACTTCCTCAGGCCAAAGCCAACCCACCTCCCACTAAGACACCTCCAGCGTCTGCA
TCTGGAAAAGCTGTGGCTGCTCCAACCAAGGGAAAACCACCTGTTCCGAACAGCACCGTCTCTGCAAGGGGCCAGCGGTCTGTGCCAGCCGCGG
GAAAAGCAGGGGCCCCAGCAACCCAAGCCCAGAAGGGTCCCGTGGCTGGCACAGGGGAGGACTCAGAGAGCAGCAGTAAAGAGGAGTCTGACAG
TGAAGAAGAGACGCCAGCCCAGATAAAACCTGTGGGGAAGACCTCTCAGGTCAGAGCTGCCTCAGCCCCTGCCAAGGAGTCTCCTAAAAAAGGA
GCCCATCCAGGAACCCCCGGCAAGACGGGATCTTCAGCTACCCAGGCCCAGCCAGGGAAGACAGAGGACTCAGACAGCAGTAGTGAAGAGTCTG
ACAGTGACACAGAGATGCCATCAGCCCAGGCGATTAAATCCCCTCCAGTTTCTGTCAACCGTAACAGTAGTCCAGCTGTCCCAGCTCCTACCCC
AGAAGGAGTCCAGGCTGTGAACACCACAAAGAAGGCCTCAGGCACCACTGCCCAGAGCTCCTCCTCTGAGAGTGAGGACGGGGACGAGGACTTG
ATTCCTGCCACACAACCCTCCACCTATGCTCTCAGAACCAGTGTGACGACGCCCGCAGCCCTCTCACGAGCAGCTTCCCAACCCAGCAAGAGTG
AGCAGTCTAGCCGGATGCCAAAAGGCAAGAAAGCAAAGGCGGCGGCGTCCGCTCAGACCAGCAGTGCCGTGGAAACACTCCCCATGATGCCTCC
CCAGAGCGCACCCATCCAGCCCAAAGCCACCAACAAGCTCGGGAAATCCAAGCTCCCTGAGAAGCAGCAGCTTGCCCCAGGCTACCCCAAGGCC
CCCAGGAGCTCGGAGGACAGCAGTGACACTTCTTCCGAGGACGAGGAGGATGCCAAGAGACCCCAGATGCCCAAGTCGGCCCACAGGCTGGATC
CAGACCCTTCCCAGAAGGAAACTGTGGTAGAGGAGACCCCTACAGAATCCAGCGAAGATGAGATGGTGGCCCCCTCACAGTCTCTCCTCTCAGG
TTACATGACTCCCGGCTTGACTGTGGCTAATTCCCAGGCTTCAAAAGCTACTCCTAGGCCAGACTCCAACTCCTTAGCTTCTTCTGCCCCGGCC
ACCAAAGACAACCCGGATGGCAAGCAGAAGTCAAAATCCCAACACGCAGCAGACACCGCACTCCCTAAAACCGGTAGGAAAGAGGCCTCCTCGG
GCTCCACGCCTCAGAAGCCCAAGAAGCTCAAGAAGAGCACCTCAAGCTCGCCGGCCCCCACACAGACACTGCCGAACAGCATCACCCAGCGCCT
CCTGGAGCAGGCCTGGCCCCTGAGCGAGGCGCAGGTGCAGGCCTCTGTGGTGAAGGTCCTGACAGAGCTTCTGGAGCAGGAACGGCTGAAGGCC
ACAGAGGCCATCAAGGAGAGTGGGAAGAAGAGCCAGAAGCGGAAGTTATCAGGGGACCTAGAAGCTGGGGCCCCAAAGAACAAGAAGAAGAAGG
AGCAGCCAGTGCCCAGGGCAAGCGCTGTTTCCCCAGAAAAGGCTCCCATGACTTCCAAGGCAAAATCAAAGCTTGACAAAGGGAGTGCTGGTGG
CAAGGGGAAGGGGTCTCCTGGCCCCCAAGGAGCCAAGGAGAAGCCCGACGGCGAGTTGCTGGGGATAAAGCTTGAGAGTGGCGAGCAGAGCGAC
CCGAAGAGCAAGTCGAAAAAGAAGAAATCCCTCAAGAAAAAAAAAGACAAGGAGAAAAAGGAAAAGAAGAAAGGAAAAAAGTCCCTGGCCAAAG
ACTCTGCCTCGCCGATCCAGAAGAAGAAAAAGAAGAAGAAGAAGTCAGCCGAGCCTGCCGTGTGA
MOUSE SEQUENCE-CODING
ATGGCCGAGGCCAGGAAGCGGCGGGAGCTGCTTCCCCTCATTTACCATCATCTGTTGCAAGCAGGCTACGTCCGCGCGGCGCGGGAAGTAAAGG
AGCAGAGCGGCCAGAAGAGTTTCCTGACTCAGCCCGTCACCCTTCTGGACATCTATACACACTGGCAACAGACCTCAGAGCTTGGCCAGAAGCA
GAAGGCAGAGGATGATGAGACCCTTCAGGCTAAGAAGTCTCGAGTGTCGGATCCTGTTAGCAGCTCAGAGAGCTCGGATCAGGAGAAGGAAGAG
GAGGCAGCAACCGAAAGGGCCAAAGCCACCCCAAGACCGACACCTGTCAATTCTGCAACCGCAGCTTTGCCATCAAAAGTAAAAGAAAAGGGAA
AGACCAAGACAGCCAACAAGACGGTGAACTCTGTGTCGCACCCTGGGTCCGGAAAGACGGTGGTCCACCTGCTCTCTGGGAAGTCACCCAAGAA
GTCAGCAGAGCCCTTGGCAAACACTGTCTTGGCCTCAGAAACTGAGGAGGAGGGCAATGCCCAAGCCCTCGGACCCACTGCCAAGTCTGGAACG
GTGTCAGCGGGCCAAGGCAGCAGTTCCAGTGAAGATTCCTCCATCTCAAGCGATGAGACAGATGTCGAGGTGAAATCTCCAGCAAAACCAGCCC
AGGCCAAAGCTTCAGCAGCCCCTGCCAAGGATCCTCCAGCAAGAACAGCCCCAGGCCCTACCAAGTTAGGGAATGTGGCGCCCACACCTGCTAA
ACCAGCCAGGGCGGCAGCGGCAGCTGCTGCTGCTGCTGTGGCTGCTGCTGCTGCTGCAGCAGCAGAAGAGTCTGAGAGCAGTGAGGAGGACTCA
GACAGTGAGGACGAGGCCCCTGCTGGTCTGCCCAGCCAGGTAAAAGCCTCTGGAAAAGGTCCCCATGTCAGAGCCGACTCGGTGTCTGCCAAGG
GGATCTCTGGGAAAGGGCCCATCTTAGCAACCCCAGGGAAGACTGGGCCTGCAGCCACCCAGGCCAAGGCAGAAAGGCCAGAGAAGGACTCGGA
AACCAGCAGTGAGGACGATTCTGATAGTGAGGATGAAATGCCAGTCACTGTGAATACTCCTCAGGCAAGGACTTCTGGGAAGAGCCCTCGGGCC
AGAGGTACCTCAGCCCCCGCCAAGGAGTCATCCCAGAAAGGGGCTCCTGCAGTCACCCCTGGAAAGGCAAGGCCTGTGGCAGCCCAGGCAGGGA
AACCAGAAGCCAAGAGCAGTGAGGAGTCAGAGAGTGACAGTGGGGAGACACCAGCTGCTGCGACTCTGACCACGAGTCCTGCCAAGGTGAAACC
TTTGGGGAAGAGCTCCCAGGTCAGACCTGTTTCCACCGTCACCCCGGGGTCATCGGGAAAAGGTGCCAACCTGCCCTGCCCTGGGAAGGTGGGG
TCAGCAGCTCTCAGGGTCCAAATGGTAAAGAAAGAAGATGTCTCGGAGAGCAGCAGTGCAGAGCTGGACAGTGACGGGCCTGGGAGCCCAGCCA
AGGCAAAGGCCTCCCTTGCGCTCCCTCAGAAGGTGAGGCCTGTGGCCACCCAGGTCAAGACTGACAGGGGCAAAGGCCACTCAGGGAGCAGTGA
GGAGTCATCTGACAGCGAAGAGGAGGCAGCACCAGCAGCCTCTGCTGCTCAGGCTAAGCCAGCTCTGGAAAAGCAGATGAAAGCTTCCTCTAGG
AAAGGCACGCCTGCATCCGCAACAGGAGCGAGCACCTCGTCCCATTGTAAGGCAGGAGCGGTGACCTCTTCAGCCAGCCTGTCATCCCCAGCTC
TGGCCAAGGGCACCCAGAGGTCAGATGTGGACTCTTCCAGTGAGTCTGAGTCAGAAGGAGCTGCTCCCAGCACCCCCAGGGTACAGGGGAAGTC
TGGGGGCAAGGGCCTCCAAGGGAAAGCTGCCTTGGGGCAAGGGGTGGCCCCAGTGCACACTCAGAAGACAGGGCCTTCGGTCAAAGCTATGGCT
CAGGAAGACTCAGAGAGCCTCGAGGAGGACTCCAGCAGTGAGGAAGAGGATGAGACCCCAGCACAGGCCACGCCCTTGGGGAGACTTCCTCAGG
CCAAAGCCAACCCACCTCCCACTAAGACACCTCCAGCGTCTGCATCTGGAAAAGCTGTGGCTGCTCCAACCAAGGGAAAACCACCTGTTCCGAA
CAGCACCGTCTCTGCAAGGGGCCAGCGGTCTGTGCCAGCCGCGGGAAAAGCAGGGGCCCCAGCAACCCAAGCCCAGAAGGGTCCCGTGGCTGGC
ACAGGGGAGGACTCAGAGAGCAGCAGTAAAGAGGAGTCTGACAGTGAAGAAGAGACGCCAGCCCAGATAAAACCTGTGGGGAAGACCTCTCAGG
TCAGAGCTGCCTCAGCCCCTGCCAAGGAGTCTCCTAAAAAAGGAGCCCATCCAGGAACCCCCGGCAAGACGGGATCTTCAGCTACCCAGGCCCA
GCCAGGGAAGACAGAGGACTCAGACAGCAGTAGTGAAGAGTCTGACAGTGACACAGAGATCCCATCAGCCCAGGCGATTAAATCCCCTCCAGTT
TCTGTCAACCGTAACAGTAGTCCAGCTGTCCCAGCTCCTACCCCAGAAGGAGTCCAGGCTGTGAACACCACAAAGAAGGCCTCAGGCACCACTG
CCCAGAGCTCCTCCTCTGAGAGTGAGGACGGGGACGAGGACTTGATTCCTGCCACACAACCCTCCACCTATGCTCTCAGAACCAGTGTGACGAC
GCCCGCAGCCCTCTCACGAGCAGCTTCCCAACCCAGCAAGAGTGAGCAGTCTAGCCGGATGCCAAAAGGCAAGAAAGCAAAGGCGGCGGCGTCC
GCTCAGACCAGCAGTGCCGTGGAAACACTCCCCATGATGCCTCCCCAGAGCGCACCCATCCAGCCCAAAGCCACCAACAAGCTCGGGAAATCCA
AGCTCCCTGAGAAGCAGCAGCTTGCCCCAGGCTACCCCAAGGCCCCCAGGAGCTCGGAGGACAGCAGTGACACTTCTTCCGAGGACGAGGAGGA
TGCCAAGAGACCCCAGATGCCCAAGTCGGCCCACAGGCTGGATCCAGACCCTTCCCAGAAGGAAACTGTGGTAGAGGAGACCCCTACAGAATCC
AGCGAAGATGAGATGGTGGCCCCCTCACAGTCTCTCCTCTCAGGTTACATGACTCCCGGCTTGACTGTGGCTAATTCCCAGGCTTCAAAAGCTA
CTCCTAGGCCAGACTCCAACTCCTTAGCTTCTTCTGCCCCGGCCACCAAAGACAACCCGGATGGCAAGCAGAAGTCAAAATCCCAACACGCAGC
AGACACCGCACTCCCTAAAACCGGTAGGAAAGAGGCCTCCTCGGGCTCCACGCCTCAGAAGCCCAAGAAGCTCAAGAAGAGCACCTCAAGCTCG
CCGGCCCCCACACAGACACTGCCGAACAGCATCACCCAGCGCCTCCTGGAGCAGGCCTGGCCCCTGAGCGAGGCGCAGGTGCAGGCCTCTGTGG
TGAAGGTCCTGACAGAGCTTCTGGAGCAGGAACGGCTGAAGGCCACAGAGGCCATCAAGGAGAGTGGGAAGAAGAGCCAGAAGCGGAAGTTATC
AGGGGACCTAGAAGCTGGGGCCCCAAAGAACAAGAAGAAGAAGGAGCAGCCAGTGCCCAGGGCAAGCGCTGTTTCCCCAGAAAAGGCTCCCATG
ACTTCCAAGGCAAAATCAAAGCTTGACAAAGGGAGTGCTGGTGGCAAGGGGAAGGGGTCTCCTGGCCCCCAAGGAGCCAAGGAGAAGCCCGACG
GCGAGTTGCTGGGGATAAAGCTTGAGAGTGGCGAGCAGAGCGACCCGAAGAGCAAGTCGAAAAAGAAGAAATCCCTCAAGAAAAAAAAAGACAA
GGAGAAAAAGGAAAAGAAGAAAGGAAAAAAGTCCCTGGCCAAAGACTCTGCCTCGCCGATCCAGAAGAAGAAAAAGAAGAAGAAGAAGTCAGCC
GAGCCTGCCGTGTGA
HUMAN SEQUENCE-GENOMIC
CAGGGAAAGGTAGACAGGTAGTTCCCTCTGCCTCTAACCTGCTGGGGTCCTTGTCGAGTCACTTCCTGTCACTGGGCCTGTTTCCTTATCAGCA
AGAGGAGTGAGCTGGGCTAGATGCTCTCCAAGGACCCTTCTAGCTCTGAGAAATAAAACCACAAAAATAAGAAATTATGCTGATGAGGCTATGA
ATTGCTACAACCCTTTTGGCAGTTAATTGGGCTTTAGCTATTAAAATAAAACTCATGCATCCCCTCTGGTCCACTTGCAATTACATCATACAGA
ATTAAAGCTCCAGTCTGCAAGGATGTATTTACTAGGATGTTTAGAGCAACACTACTCTATTAGCCAAAAAACTAGAGGCGAATAACCTGTATCA
TATCATCTCCCATCTCTGTCTGTTGATGGCCCTACTGAGGGACTGGTTATGAGCCCTCTGGCAGCTGGAGCCTGTCTTGAGCCTCAGTTTCCTC
ATCTACAACCATTTGACATGCTTTATAGGATGTTGTGAGGTTTTGATTGGTTAGTGTTAGAGCCTGGAAGCACAGCTCCAGCTCACTGCACACA
CTCAACAAATGATAGCTGTTACTATTACTCAAAAGAGTGTGGCAGATCCCAGCATCTAGAGAAATAGCTCTGCTGTATTGTTATTAGGTGAAAA
AAGAGAGTTGCAGAGTTGAGTGTTTAGTATGATTCCCTTTTGTAAGAAAAGAAGCAAAGAAGCCAGGTGCAGTGGCTCATGCCTGTAATCCCAG
CACTTTGAGGGGCTGAGGTGGGTGGATCATCTGAGGCCAGGAGTTCAAGACCAGCCTGGCCAACATGGTGAAACCCATCTCTACTAAAAATACA
AAAATTAGCTGGGCATGGTGGCACATGCCTGTAGTCCCAGCTACTCGGGAGGCTGAGGCAGGAGAATCATTTGAACCCAGAAGGCAGAGGTTGC
AGTGAACAGAGATCATGCCACTGCACTCCAGCCTGGGCAACAGAGCGAGACTCCGTTTCAAAAAAAAGAAAAGAAAAGAAAAAGAAAAAAGAAA
AGAAGTAAAGAGAAGTGAAAAAGGAAAAGGAGAAGGAGGACTAAACCTTATGAAGGATGTCTGTGTTTGTCTGGGCAGAGAAAGGATGTGAAGG
GATTGAATACCAGGCTTCTTTCTGATTGGGAAATGAAGAGGCTCTAGTGTGGAGAAGTGGGCAGGGAATGGCTAAGAGGGAGGGAAAAATGATG
GACTTTGTCTTAATATATCTCTGAATTGTTTAACTTGTTACTTCAGTAACAACTTGTAAACCTTTAAAAGCTCCTGTGAGATTAAAGATTTCGT
GTCATTTCACGAAAGGGAAGGAAGTCTTGGGAAATCTACTGGCCAAAATGACTTGGCCCAGGGAAAACCTGTTGTTACAATGGATGAAGGGAGC
TACTGGGAAGGGCACCCTTCCCAGATGGGCCCTTCTCTTAAGAGTTCCACTACTGGAAGGGTCTTTGTTCAACAGCTTCATTTAACAGAAGGTA
ATGCTGAGGACCAGAGAGGGCAAGAGACTGGCCCAAGGTCACACAGCTGAGCCAGGTGAAAAGGGCCTGACTCCTGACAGTAACACAGGGGAGA
GGCCCAGTAACCTTTCCCCTGTATCGCTGCTACCTCCCACCTCTGTGTCAGAGCCATTCCTGCTGGGAGAGACAGGTAGAGTGCCCCATCTGTT
GAGCTTCCTTCTCTTCTGTTGTTCATTCTTCCTTCATTCATTTCCTAAGTGACCTCGCCGTGCCCTGCATGGGAATCCAAGGTGACTAAAGCAT
GCTGCTTCCCCAAGAGGCACTCGCAGTTCAGTGGTCTGGGAGCATCTGGCTTCTACCACTAACAAAGGCATTTTCTCTTACTGGTAGAGAAATG
CTCTGAGGCTAATTTTTTTTTTAATGCACTGTTTTGTTTGTTGCTGTCTTAATAGTTGCCATGTTATATTTTATCAGTTTAATGTTCTAATTTT
GTGCTTGGGCCCTGAGGCAATTCAGTGAAGGGTATGATTTGGAAAATGAGGGAAATAAAGAAATTGTCCACAGAAGAGACAGCCTCCCAGGAGC
CGAGGACAAAGCATAAGTGCCGACAATGAATTTTATTAGGAGCAAAACAAAAACAACCTGTTGCCAGACAGAAGGTATTAGACAATCCTGGTGT
GGTTTTGTTTTCGTTCTTTAATTTAATTCCAGTTCTGCATTTCTTCCAGGGTCAGCCAAGGATGCACAATCCAGTTCCTTCCCCACTGCCTTCC
AAAGCCTTCCACATTCCTGCCTCCAGGCCTAAACTCAGTGAGCACCTTGCCTGCCCTGCCCTCCCTTCTTCTTCAGGAGAGGTCTGTAACCATT
CCTGCACTTGCCAATACTTATTAAGTGCCTGCTGTATGCAGGCACTGTGCAAAATGTTGGAAATAATGCAACCAACAAGGCAAAGGTAAGTCTG
CCTTCAGGGAGTTTACATTCTACTAAGGCATGCATTCTCAAATGGGGCCTTGATGCCCTCGAGGAAATGAAAACTGGTTCCTGGAGGTGTTGGT
GGAAAAAAATCTTAGATCTTACAATGGTTTGTATGCTCATCATGTACATTCATCACCCATCCAGCCACCCAAGCAGAAACCTGGGTGTCACCAC
CAGTTCCAGCTCCCAGCCCTCTCTGATCTCAGGGGTCCCTCATAAGGCCTGGCCCGTGAAGGATTCCACTCTCTCCCTGCAAACATCCATCTGC
CTTGCTTCCCTCGCTACCCAGTGTAGATCCCAGGACCCAGAATTTTAGCATCTCTTTGCCAATACTTTAGACTCCTGTGCCCTTTTTTCCCCAC
CCATCTGGCAAAGCCCTAGGCCTGGATAAATCCCACTATGTACATACATCTCCCCAGCCTGCAGCTGGGTAGGTGAGCCCTGATGGAGGAATTC
ACTCAGCAGGACAGGACGAGACACACATACACTAAGGACCACCAACCTCAACTGGGGAGCCAAGGCTGTCAGGCAATTGGGGATTTTGTGGGCA
AGTTGCTTCTCAACACTCTGAATGATCATTCTTTCAAACTCCTCACCACCTCTTCTTCACTCTCAGCAGAAGACCTTGCCTCCCACTTTGCAGA
AAAAATAGAAGCTCAAAATGAAATCTCCCTTAACATCCCACTATAAACCTACATCTACATCTGCACCCATCCTCTCTTTGTTCCTGCTTTTGGG
TTCTAGAGAACTCATCCCTCCTCCTCCCCAAGACGAACCCCCCCAACCCACATGCCTGATCCCAGCCCCTCGCACATGCTTGAGAGAACTTTAC
TCGCTGATTATCTCCCCTATTTCTTACACTCTTGGTTAACCTCTCTCTCCTGGATCTGCCATTCACTTTTAAACAAATCTCAGGTTTCTTCTAC
CTTGAAACACACCTCCCCTAACATACATATTGGCCCTAGCACTTTATCTTTCTCGTCCTTTTTATACAAAAACTTCTCGAAAGACCCACTGCCT
CCCTCATCCCTCCTGCACTCATCAGACCACTGCCATCTGACCCCCTCCATCATGCCACACATAAATAGCCCTCCCTAAGTCCCCAACAATCTCC
ACGCCTGTCCATAAACCAAAGCCCAGGGCTGCACCCTCCTCCTATCTGACCTCTCAGAGCATTCCCTCCATCAAGTCTTCCCAGCTTTCTAAAA
TGTCTCCCTTGGCCTCCTTCCATAAGCCCACTCTCCTGGGCTTTCTCCAAGCTGTCCAGCCACTCCTGATCAGCCTTTGCAGGCAGATCCTTTT
GTACACAGCCCTAATCTGTAGATGTACCTCAAAACTTCATCCTGGGCCATCTCTCTTCTCTCTCCACATTCTTCTCCCTGGGTAAGCTTCTCCT
CATCCACAGCCGCAATTATCATCTGTCTATCGACAACTTCTCATCTTCATCTCTAGCCCAGACCTCCACGCCACGTCCAAACCCAGGTTTCCAA
GAACCTTCTCAACATCTGCATGTGGTGACTGTAAGGCATTTCAAGCTCAAGATGTCTAACATAGAATATATACTTTCACCTCTCTGTCCACATC
CTCCTCTCCTCAATCAAACCACCCCCCTGTCTCAGTCCTCTTCTAGTTTTTTCTATCTCAGTGAAAAATACTGCCTACTTGTTTGCACAGAAAC
CTCAAGGTCCATTCTGACATTGCCTTTTCCTTCCCCTCCCATTCCAACACACCTCTGTGAAAACTTTAGTTCCTGATTAACTCTGGGAACCTTC
CACTCTCTTCAACTCCACTGCCACCAGCACCCTCCCCGCTGCCCCCGCCACCTACCACCCGCCCCCCCCCCCCCCACATACACTGTCTTCTCCC
TGGATGCCTGCCTGCACTCCTAACCCTGACTTGTCTTCCCACATCTACTCATACCCACCAGACACACTCCACTCTCCACTGGGCAGGCACACAG
GTCACAGTAAAATTCAGATTTCATCCTGTACCTCTTGGGCTTACACCGTCAGTGGCTTCCAATGGCTCTTAGGATGGAGGAAAAAAAACCCTGA
TGTGGCCAATCCTTCACATGGAGTCTGCCCTCTGCCCACCTTCTACACTCCCCAGTCCCCCTGCTCTCCCTCCGGACTATGCTACAAAGGCCTT
TTTGTGTCTATTCTCTCAGCTGTACCTTCTGCCCTAGGCCCTTTGCACAGGTTCTTCCCTCTGCTCAGGTTTNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNTTTAGTATGTGTCTCCTCCGTTAGA
CTGTAAGCTCTATTGGAGTAGAGACAATTCCATCACTAGACTGTAAGCTCTATAGGAGTAGAGACTATTCCATCCCCAATGGTAGGTACTCAAT
AAATATTTGCTCAAGAAAATGGGAAATGTTGAATGTGTACAGACACATAACATTTTCCATTCAAGTTCAAGACAAAGATAACCACATACCTGGA
CCTCCTAGGGTTAGGGACTCCTGCGCCCGTAAGAAACATGCTGCCCTCAGATTCTCAATGGGTCGGTGAAGTTAAGAACAGCTCACTCTAACAT
TCTCCAGCCTACCTCACCTTTTCCCTGCCATCCTTCCCTCAACAACTACAACAGCCTCACATTTAGTATTAATGGTATGTGCTTTGGTTTTAGC
AATCTTTTAAAAACACAAAACAGGGTCAACCTCCTCGGAAAAGCCAGTCAGAGACTATCTAATGCCCTCAGAGCAAAGTCCAAACTCTGTTGGG
GGTGGGGGAGTTGTAGGCCTTGTCTACTTCTTCATCCTCATGTCCCCTCATTTGTTCCCTCTTCCCCACCTCACCCCACCCCACCTGGACAGCC
CAGCAGCGCAGACTAGGGGTTCTGGGTTTAGGGTTCCCAGGCAATAGCAGGGTGTTTTCTCATGCCTAGACTCATGCAGTGCCCTCTGCCAGTC
CTCTCCTTTGCACCCAGCAATTTCGGGTGTCTTTATGCCTCAGCTTAGATATCTCTCTGGGGTTCGGTGCTCCTCCTTGGGGCGCCCATAGGCT
GCATCTGTCCCTGACAAGGCACACAAGATATTGAACTATAGTTGCTTGGTTACGGACAGTCGTCTCTGTGAGCTTTGAGGGCAACTGCCATGTA
TTATGCATCTTTTGAATCCCCCTAGCCTAGCCGGGTGCTAGCCCAAGTTAGGTGCTTATTACAGATTTGTTCAGTTAGCGAATGAATCAATGAA
TAAACTGGCAATTCGTGTCTGGTTCATGGGCACCAAAAACCGTGCGAAATGGATAAATGAGCAGGTAATGGCAGTACAAAGTTCGGATCTAGAG
AAAGATCCAGATTAGGGAGTTCAGAGATGAGTAAAACGCAGACCCAGCCATCAGAGAATTAACAAGGAAGATAAATCTCTTCAAGAAGCCAGCC
GGAAGGATTAAAAGAGAGTCGTCCAAGTCCCGGCCGGCCCCGGGGGCGGGTCCCTGTCTAGCCCCGCATGCTCCGCGCCAATGGGCGGTATTGT
TGATCACGAGTCTCCGCCCCCTTCGCCTTGAGGGGCGGGGCCGGCCACTCCCGGAGAGGGGACTACGTTTCCCGGCGCGCCGCGCGGCCGGAGC
GAAAGAGGAGCCGGAAGTGGGGCGCGCGAGGTCTAAGGGCGCGAGGGAAGTGGCGGGCGGGGACTAAGGCGGGGCGTGCAGGTAGCCGGCCGGC
CGGGGGTCGCGGGTATGGCCGAGGCCAGGAAGCGGCGGGAGCTACTTCCCCTGATCTACCACCATCTGCTGCGGGCTGGCTATGTGCGTGCGGC
GCGGGAAGTGAAGGAGCAGAGCGGCCAGGTAAGCGTTCGTGGGCCGTGTGCGAGGGCCGCGTGCAAGATGTGGAGATCAGCGGCCCGCGGCCCG
CGCCCCGTCCCCAGGCGACCCGGCAGGCGCCCGGAGCCGGGTCCCGCAGTGCTCGACGGCGCGGCCAGGGGTACCGGAGGAGCCGCAATCTCTG
CCTTCCCACTGCGACTTCAGTTCCCCTGGGCCTCACTTTCCTCATCCGGGCAGTGGGTTGGGCCAGAAAGTGTGCTCGCAGGGGCCGAACTTTG
TAATTCCCGCTTCCCTTACCTCCACGCCCTTCCTGGTAGCGGGTATTTTAAGTTTCCTAAGTCTCCCGCCACGTGGCTAGGCTCTGCGCGGCCC
CCCCTGGGGCAAGGAGGTTGCTGCGAGTCTCGGGGGTGGATTGCGACCAGCCCCTCGGGGGAGCGCCTGGAAAAGGGACCCTACAGCCTCTTAG
TTGAGCTGGAACTCAGGCCTTTAGCGATTGTCTGTACTGAGACGGTCAGCTTTGGTACTCGAACTCGTGGTACAGTCATTTCTTACTTGATTAA
TTCAACTGTTAATCCGGTACTCAGGGTGCTTCCTGGTGTTCGAATGGTGAGCACCATCTCTCAGCCCTCCTGGAGCTTACACTCTAGTAAGGTA
GACAAATCAAAGAATCACAAAAGTGACTATATGCTTAGCACCGCAATAAGTGTTAAGAATGAGAGGGATGGGCCGGGCACGGTAGCTCACGCCT
GTAATCCCAGCACTTAGGGAGGCCGAGACTAGCGGATCACCTGAGGTCAGAAGTTCGAGACCAGCCTGGCCAACATGGTGAAACACCGTTTCTA
CTAAAAATACAAAAATTAGCTGGGCATGTTGGCGCACGCCTGTAATCCTGGCTACTCATGTGGCTGAGGCACAAGAATTGCTTGAACCCGGGAG
GCAGAGGTTGCAATGAGTCAAGATCGTGCCACTGCACTCCAGCCTGGGCGATAAAGCGAGACTCTGCCTCAAAAAAAAAAGAGAGATGGATGGC
AGATTGCCCTGGTCAGGAATAGCTGAGGTGTAAATGCCTTGTGGTGGGAGGGAACTTGTGTACACACTGGCCTGGAGAAAGTCCACTCTCTTGA
GCCCAGGAATTTGAGGATACAGTATGAGCCATGATCATGCCACTGCACTCCAGCCTGGGCAACAGAGCAAGACACTGTCTCAAAAAAAAATTTT
TTTTAAGTCCATAGCAGGCCAGGTACGGTAGCTCACGCCTGTAATCCCAGCACTTTGGGAGGCCGAGGCAGGCGGATCACTTGAGGCCAGGAGT
TCAAAACCAGCCTGACCGACATGGTGAAACCCTGTCTCTACTAAAAATGCAAAAATTAGCCATGCATGGTGGCATGCACCTGTAGTCCCAGTTA
CTCGGGAGGCTGAGTCAGGAGAATCACTTGAACCCAGGAGGCAGAGGTTGCACTGAGCTGAGACTGAGCCACTGCACTCCATCCTGGGTGACAA
GGCGAGACTCCATCTCAAAAAAAAAAAAAACGTCCATAGTGGCTGAAGTGTGAAGTACAAGGGCCAGATGCCCCTGGGGAGATGGAGGCAGGGG
CTGGCTGGAGTGGTCTGGTGGGCAGTTTGATCTTCCTTGGAAGAGCAGTGGTAAACCAATGGAGAGTTTTGAACTGGTGAGACAGTATCCTAAG
TAGTCAGATCCCTTTCCCCTGTGGTGTAGGCTCTGCTCCTAATGCTTGCTCTCAGAACCTCCTGGAGGGACTAAAGGGTGATGGGCCCTAAGAC
GTTATGTATGCTTTTCCCAGTTTCTCTGAACCTCACTTGCTCTTCCTACCGCAGCTATGGTACTTCACAGATAAGGGGAGCAAGAGATAGTACC
CATCTTCATTGGAGCAAAATATCCTGCCGTCATGGAGCACACATTCTAGTAGGAGCAGACAGGTGACAAATAGAAAAAGTTTAATAAGTTAATG
ATATAGTATATCCAATGATAGTTGGTGCTTGCTATAAAAGGCAGGCAAGGGGGATAAGGGATGTCAGACTTCGATTATAAGTAGGGTAGTCAAG
GAAGTCTCATTGAGAAGGTGTCATTTGGGCAGAGACCTGAAGGAAATGAGGGAGTGAGCTGTGTAGTTAACAGGGGACAGAACATTCCAGACAA
GAAGAATAGCAAGGCCGCGTTAGAGGCAGGGTGGCCTGACACATTTGAGGACAGCAAGGAGGACACTTTGCCTGAGGTGGAGCAGAGGGAAACT
GACTCTTGAAATCTTAGTGTTACCGGCTCACTGCTGGGCACTGACAGATGGTCTTGTTCAAACCAGATAGCAGCCTGCAAGGTAGGTATCGCTG
TTGTCCCCATGTTAAGAAGAAATCTGAAGTTCGAAGACGTGACGAGATTAGTTCAGTCCACCCGTGCCCCACATTGCTTAGCCTCATTAGAGTA
CTTGATATCCTGGCTTATTGACATTTACAGAGACATTGGGCCCTCTGGCCTCTGCTCCAAAGGCCTGGATTCCACCTGGTTCCCTCTGCTTCCA
CCAAGGGACTGGTACCATTTGGGCCCAGTCTTCTCCCAGCAGACTAGTAGACTGTGGTCATGCCACCTGGATGCCTCAGGTGTCTGGGCTGTGA
TTCGTGCTTTGGGAAGATTGGCTGGCCACGTCTTTGAAAGCTTTTTTAAAGGTCAAAGGTGGGGAAAACCCTGTCTCATGGCCTCTAGGCATGG
ATAGTGCCAGAGAGGCTGGGAGCTGGATGAATGCTAGCTTGGAACATTCCACCTCTGGCCTTTCAAAGGGGTTGTCTAAAAAGGTTTTAGTTGG
GATTGGTTGTTCTTCTTGTCTCTGGAAGGTTTGGGACAGAACTGGCAGGGCCAAGCAGACATACTTCCCAGAGCAAAAAGACCCTCTTTTTTTT
TTTTTTTTTTTTTAAAGAAAGGGTCTTGCTCTGTTGCCCAGGCTGGAGTGCGGTGGCACAAGCATGGCTCACTGCAGCCTCGATATCCTGGGCT
CAAGTGATCCTCCCACCTCAGCCTCCTGAGTAGCCGGGACCACCAGTGCATGCCACCATTCTCAGCTAATTTTTAAATTTTTTTATAGAGACAG
TCTCACTATGTTGCCCAGACTGGTCTTGAACTCCTGGGCTCAAGTGATCTTCCTGCCTCAGCCTCCCAAAGTGCTGAGATTACAGGTATGAGCC
ACCATGCCCAGCCAAAAAGACCCTCTTCTGAACCACCTGTCTATACAAGTCATGAGTTTGGGGAGATCTGGGCCCAAGAAGGATCCTTACTGTG
CTGGGGATTAATTGTGGCTTTCTCTTTACCTCTCTGCAGAAGTGTTTCCTGGCTCAGCCCGTAACCCTTCTGGACATCTATACACACTGGCAAC
AGTAAGTGGTGGGGCCTATAGGGTGGAGTAGGGACGGACACCCCAAGCAACTCAGCTTGGAATAAGCTGGGATACCTATCTGGTCTAAGATCTG
TCCCCATAGCCCACTGTGGACACCATTGTTGCCTCCAGAAAGCCAGCTCACATCACATGGATGGGTATTTCTGTGTGGATATCTGGTAGTGGGA
GGGGACATTGAGAGCTGTGATCTCTTAAGAACAAGAGCTTCTCACCAGCTTGTCAGTTGCTCAGCAAACGTATATTGAGCACCTCCTGAAAGCC
AAGTCCTGTTCTTACTTAGTATGCAAATATTGTCACAAGTGAGACAGACATGGTTCCTTCTTTCCTGCAGCTGACATTGTAGCCAAGGAGGTAG
GCATAAGTCAAAGAATGAAACAAATGAATGTTAAATTACAGTGGTAGTAAGGATTACAAAAGAGAGGAATCTGTTGCCACAGCCACATAAATGG
GTAAGTCTGGCTTCATCTGGGGGTCCAGGGGAAGCTTCCCTGAGGTGATCTTTGACTTGAGAGCTATAGGAATTGGAGGAGTGAACTATGCAGT
GGGTAGAGAGTGGCAGGTGGAGTGTACCTGCCAGTGGAAATTGCTAATGCTAAGTCCTGTGGCTGGTGGACAGTACAGATGGGGCTAGGTCATC
GCATGGATAGCCATGCATGGGCTGGTTATCCATGGTGAGGAATTTTGTCTTTATCCAAGAGCAGTGGGGCCATGGTAGGGTGAAAAAGTGAGGA
GATTGCTCTGCACCATGGTGAGACTGGTGGGGCCATGGTCACCATCGTCCAGGCAAGAGCTGATGGGGCTTGACACAGGGAGAACAGGGTAGAA
AGTGGATTCCCAAGAATCATAGGGATGAAACCAGGGCCTGTGGATGGGTGGGAGAGGAAGTATCTTATTGACTCCACATCTAACTTGTGTACCC
ATGCATAGAGCTTGAAGGGAACTCAGTTTGGAGGGGAAAGATTTCAATGACATGTTGAGTGTGAGATGCCTTTTAAACATGTAAAATTTATGTA
CTCTTCAAGGAAGGGGGAATCATCCAAGGATATATCAACAGAGGAATGGTTGGAAAATGGAGAGAGTAATTGTGTTCTCTTCATGCACTGGCCT
GACCACAGCTGACTAAGGGAGTGAAGGACATGCAGACATATCCCATGAGGAATGGTAGAGAGTACTGAATTGCCTTGGATGGAAAGGAGGCAGG
AGAGGGGTGTGAGGGACCACTGCACATCTTTGAGGACCAAGAATAGTGCGTTCTCTGAGGCCCCGGAGACAGAAGTGGGCCCCAAATGTACCTG
CTTCAGGAAGACAGGTTTCAGCTTCTGAATATAATGACATCAGAGCTATCTAAAAATGAGGTGGGCTAGGTGGAAGTGGTGGGCTCCCTGTCAT
TAGAGGTGTGTAAACAAAGGCAAAAATGGTGTGGAAGGGATTACAGCCTCTGGTGGAGAGCTAAGGTCATTGAAAACCCTGCCAACCCTCAGTC
CTCTGCTGCTAAGGAGAGCTTAGAGCACAGGGCACAGCCCACACTTCCTCACCCTGACTTGCCCAGGTGAGTGGGTGTTATTCGTAGTACTTCC
TTCCCCCAAAAAACTGCTTCAGTGAGCTACTCTGCACACTCTTCCCTGCCACTGGCCCCTTCTATGGGTGAGGGGGACAAGGACCTCAAACCCA
CGAAAGGCAGCCTAGCCTAGCAATTCCCTGCAGAGTTCAAACCCAGCAACTAGCTGTATGTATGACCTTGAGTGGATTTCTTGGCCTCCCTGAG
TCTGTCTTCACCTATAAAATGAAAATAATGATGGCAATTATCTCATTGCATTGTGAGGGTTAGACACAGTGCTCCATATAAAGTGCTAAAACTA
CCGGTTGGAGGCTCCCCGACTTCCTGCACCCGCCTCAGTATTTATTAAGCATCTACTACAACCCAGCAATATTCTTGATATAGCTGGGAACAAA
CCAGAGAAAAATCCCTGCTCAACATTTTTGTTGGGGGAAAAGAGGCAGAATAAATTGATACATGTAAAAATGAAACCTGTTTTTAACAGGAGAA
TCCTGACAAGTGGCTTTCCTGGGAACAAGGGACCCTACTATCACATCCCAAACCATGACAGAGCAGAGTCCATCGCAGCTGGGAAACCTGGGGC
CTAGACAGTGACTCCCTGGGATGGGGGTCAATGGGCGTGAGACTTCAAAGTCCAGGGCCTCAGCCATCTGGGTGCTTATCCTACCCATGCCCTA
GACTCACTTTGCCATGAGCAAGTCACTTAGTTGTTCAGTTTTTGAGACATGTGTGAGGCACTGAGGATACTGTAGTGAGCTGCCAGCTGCCTTC
CTCCCTGGGCCTTTCTGTGTAGGGGAGGAGTCTAGGAAGTAAGTAAAATAACAGGAGATAGAGAAAAGCTATGAAGAAAATGAAACAGGATCAC
AGGGCAGAGCATGCCTTGGAAGAGGGGCAATCTTCGAGGGCCTCTCTTGGGGGGCTACATTAAAGTTGAGACGGGAAGGACGAGGACCTGGGCA
GAGTCTTCCAAGAAGGCACAGTACACACAGCATGTTCTGCAGGGGGAAGAGCCTGGGCGGTTCTGTGAGGTGCCTGCATGGGTCAGCTCCTATC
ACTTAAAGACTGGTAGCTGCTGCAGCTGTTATATAACATTATCCAGCATTCCTGATTATTTTAAAGACTAATCAAGAATTGTAAAAGCACTTTA
TGAAGTGAGTGAAAATGCAGAATACAAAATTGTGCCTATACTGTGTTTTCACCACTGTTTAGATTCTATGCACATTGCCTTTAAGAGCTGGAAG
GATGCGGGAAGGTCTGTCTAGTCAATTCTTGTGGAGTTGTTCTGTCACCCAGTTGGTATAGACAGTCACCCTTGTCCTGCAGAACCTCAGAGCT
TGGTCGGAAGCGGAAGGCAGAGGAAGATGCGGCACTGCAAGCTAAGAAAACCCGTGTGTCAGACCCCATCAGCACCTCGGAGAGCTCGGAAGAG
GAGGAAGAAGCAGAAGCCGAAACCGCCAAAGCCAGTAAGAGCCTTGCAGCTTTGGGAACAGGCTATGGAATATTGATTGTTCTAGGGTAGAGTC
TACCTCCAGCTTCTTCTCTTATCACTAGAAGACCTAAAAGACCCTGGGGAGGAGATTGGGAGGGCACCACATATCTTTGGCAGTATTTGAGCCC
TTCCTCCGTGCCAGGCATTATATCTCCATGCTCTGGGGGTGCAAGGCCGAACCAAAACAACCAGATGGGCCCTTCCAGTCTCATTTGGTGTATG
GGGCCCAGGAGCTGCCCTATTACAGGCTCCCAGGTGATTCTTATTAGCTGGAAAGAAGACACACGTTCAGAAGTACTGCTGGAGGGATTTCAGA
AAGCAGCTAGCGGACACATTTTCTGTGTGACCTGTGGGATGCTTTAGGAATTGCTGATCTTCATGTTCGTTTAGAATTGTACCCATTAGCACTG
GAACGCTGTACTGGGCACTGGTTTTACACCGTTTGCAGGAGTAACTCAGTCAAAGCTTTTATAGCCCCTTCTTCTCCTAAGGTCCCAGGGCTCC
TTGTACTTTCCCCAACAGCAGCCATAACTCTACAGAGGAATTGCTGTGTCTCCTTTGCATCGTATTATCTTCTTTTGCTTACATAGAAGACACA
TTTCTTAATACTTATTTTTTATTTTTCAAGACAAAGTCTTGCCATGTTGTCCAGGCTGGTCTTGAACTCCTGAGCTCAAGTGATCCTCCTGCCT
TAGCCTCTTGAGTAGGTGGAATTCCATGCACATACCACCATGCCCAGCTTCCTGAGTCTGCTTTTAAAGTGTTTGAGATCATACTAGGTATATA
TTTTATTATTTTTTTGAGACGGGGTTTCACTCTTGTCACCCAGGCTGGAGCACAATGGCGTGATGTCAGCTCAGGGCAACCCCCGCCTCCTGGG
TTCAAGTGATTCTCCTGCCTCAGCCTCCCGAGTAGCTGGGACTACAGGCATGCACCACTACTGTCAGCTAATTTTGTGGGTTTTCTTTTTCTTT
CTTTTTTTTTTTTTTTTTTTTAATAGAGACGGGGTTTCACAATGTTAGCCAGACTAGTCTCAGGTGATCCGCCCACCTTGGCCTCCAAAAGGGC
TGGGATTACAGGCATGAGCCACTGCGCCCTGGATATATATTATATATCGCTTAATTTTACATTATAAATGCAAAACGAGTTCACTTTGAACTGT
AGAAGAGCATATAGAAGAAAACTTAAAGGTTTTTTGTTGTCCTACCCTGTTCCCTATCATTCCTAAGTGCATCTCCTCATAGGTAACCTTTAAA
ACTTCCTCCTTTGCCTCTATATTCATTCAAACATGTATTCTTACATGCATATATACAGACCTGCATGCTATTTGAAACACAAATGGTAAAATTT
CTTACTGATTGGCTATTTGGTATTAATCAATTTTATAAGCTGTGATAATGGAGGTGTAATTACATTTTTTTGTTTGTTTGTTTGGGGCTTTTTT
TGATATGAGGTCTTGCCATGTTGCCTAGACTGGCCTTGAACTCCTGGCCTCAAGTGATCCTCCTGCCTCAGCCTCCGGGTATGTAATTTTATTT
TTTTAAAAGTCTTCACTGGGTTGGGCATGGTGGCTCACACCCACCGGTAATCCCAGCACTTTGGGAGGTCAAGGTAGAAGGATCACTTGAGCCC
AGGAGTTGGAGACCCGCCTGGGCAACATGGCAAAACCCTGTATCTACAAAAAATTTAAAATTTAGCCAGGCATGGTGGCGTGTGCTTGTAGTTT
CAGCTGCCTAGGAGGCCTGAGGTGGGAGGATTACTTGAGCCCAGGAGGTCAAGGCTACAGTGAGCCATGACTGTGCCTGCACTCCAGCCTGGGC
AACAGAGCAAGACTGTCTCAAAAAAAAAAAAAAAAGGAAGAAGAGTCCTCGTTGGCATCCTGGGCAACATAGTGAGACTCCGTCTCTGCAAAAA
ATAAGAAAATTAGCTGGGCATGGTGGCATGCACTGGTGGTCCCAGCTACTCAGGAGGCTGAGATAGGAGAATCATGTGAGTCCAGGCATTGGAG
GTTGCAGTGAGCTATGAATGTGCCACTGTACTCCAGCCTGGGCAGCAGAGCAAGACCCTGTCTCAAAAAAAATTTAAAAACGCATCCTCATTGA
GAGAGAAACATGCTGAAGTATTTGTGGGCAACGTGGTAGGATCTATGGCATTTTCTTCAAAGTAATGCTTGATGGGGAAGGGTGAGTGAGATAA
TACATGAGATGAAAAGATTGGTCACAAGTTGTTAATGGTTGGAGCTGCCTGGTTCTTCCTTTCTATTTACTACTTAAAAATTTTCCATAATTAC
AGAAAGAGAGAGATGGAGTGAAGTGCCAGGAAAAGAATTAGATCATGCCATTCATCTCAGCAACATTCTTCTTAACAAAAAGAAATCTGAGATT
TAAGGAGACAATACCCTGGTATAAAAGCACTGAGAAGCTGGGTGCGGTGGCTCATGCCTGTAATCCCAGCACTTTGGGAGGCCGAGGTGGGCAG
ATCACGAGGTCAGGAGATCAAGACCATCCTAGCTAACACGGTGAAACCCCGTCTCTATTAAAAATACAAAAAATTAGCCAGGCATGGTGGTGGA
CGTCTGTAGTCCCAGCTACTCAGGAGGCTGAGGCAGGAGAATAGTGTGAACCCAGGAGGCGGAGCTTGCAGTGAGCCGAGATTGTGCCACTGCA
CTCCAGCCTGGGCGACAGAGCAGGACTCTGTCCCAGTAAATAAATAAGCACTGAGAATCAGTGAATATCGATATCTAACGGGGCCCAGGTGGCA
TGGTTCAGCCACCTCTCCACTAAGAAGCCATTTGAATTCCAGGCAACTTTCTCTGTGGACCTGAGGCTGGCTGCCTGCCTGACCACTGCCCCTT
CTTGCCGAACAGCTTTCAGGGCTACAGTGTGGTGGCCTGTGTGAATGGTGCCCATTGCAGTGTGCAGTGCCCAGTAGGCAGCAAGAATGTGGGC
CAGTCCTTTTCCCTCCAGGGGCCTCCACGTCTCATGCTGCGGGGGAGTTGGGAATCCATCAAGGATGCAGACACATAGCTTCATGTTACCATCC
TCTTCTCTCTTGCTCCTGGTGGACCTGGCTGATGCATCACAGAGCTCATTCCTGCAAGTCTGGGCTCAGCCTCAGATCCTCATGCCAGCACCAG
GCAGCCAATACCAATAGAATTGTTAGGTGAGATGAAACCTGTTTGCCATTCATAGATGAGAAAAGCTCATCTGGCTCCTTTAGCAGATGTTTGT
TTCTTGCAGCCCCAAGACTAGCATCTACCAACTCCTCAGTCCTGGGGGCGGACTTGCCATCAAGCATGAAAGAAAAAGCCAAGGTGAGTGCGAC
TGCCTTCCAAGCTATTGCCTATGGGATTTAAGGTGCCTGGTAGGGGACAGTCTTACATGTCAGAGAAGGATAGGGTTGTGAGTAATTGCCCCAC
CTGGATTCAGAGCTCAACCTGTGTCACCAGTTGTGGGATCTTAGTCCCTTCACCTCTCTGGGCCTCAGTTTACTTTTCTATAAAGATAAGGATG
ATGCCTGCTGCAGTGGGCCACTGGGCACATTCTACACCTGGGGTAGGCGATTTCTTTCCTCTTTCATGCCTGCGTTCACCCGTTCACTCAACAG
ATCTTGACTAATACCTGTTATGTTCCAGGCACTGTGCTAGGTGTGGAGGCACCGTAGTGATCAAGACATCTGTGGTTCTGAAACTCTAAAAGCA
TTTTCCAAACTGGAAACTGCTCAAAAAACAAGGTGTCATTTTCTAATAATCCAGAAAGCAGAGTTAGATACATACATGTATAAAGAATTGTGCC
TCTGCGATTGCCTGGAGCATTAGCAACAATGTCTCAAAGTAATTATGAAGTAATGATAAGATCAACAACAAACTGCTACTTACCACTTAGTAAA
TAACTACCATGTCCCAAGAACTGGGATTCTGTTTTCTCAACACCTCTGGTGAGAGGAGGTGCTGTTTTTAACCTCACTTTAAAGATTGGGAAAC
AGATTGAAGGGGTAGTTTACCCAAACTCACACAGCTAAGAAGTGGTAAGATTGGGTTCAGATGCAAGTGGCCTGTGCTCTTAGTTCACCATGCC
ATACCAGATGTGTCTGTCACTCTTGTTCTCTGTAGGCAGAGACAGAGAAAGCTGGCAAGACTGGGAATTCCATGCCACACCCTGCCACTGGGAA
GACGGTGGCCAACCTTCTTTCTGGGAAGTCTCCCAGGAAGTCAGCAGAGCCCTCAGCAAATACTACGTTGGTCTCAGAAACTGAGGAGGAGGGC
AGCGTCCCGGCCTTTGGAGCTGCTGCCAAGCCTGGTAAGAAGTCCCCACCTCTAGGAACCTAGTCCCCAGAACTTGGGCTGGTGAGGCAGGCCT
AGGCATTGCTTTAAGTTCTGGGACATTTCTGGAATCAAAAGTTTGTCTCCAGGACAGCCAGGTTCCTGCATCTTAGGACTTTTCTCATCACCTA
GACGAGGGAGGGTCAGCTTACAGGGACCTGGCCTCAGGCTGAACAGCCCACCTGGTGGTTGGAGCATTTTCTGCTTTATGACTCCCTGGTGAGT
TACAGGGACAACTGGCAGCTCAGAGACTAAACTGAAAGGCCTGATTTGCCTCTGTTTCCTATGTAGAGTAGACATCTGTCTGGCCTGCATCAGT
GGATATGATGGTGGATGGAGGTTCTGTAGACTGTCAGGTGCTGGTGCATATTTGTTGGGTCCCCTCACAGCCCTTCCCAGGGCCTCAGAGATGT
GTGGGAACATAGGGATGGTGATGGTACCCACCTCAGAAGGGGGCTTCACAGCTCAGTGCATGTGAGGCACACGAGGGGTGAGCGCTCAGCACCT
GGCTTTGATGAGCAGCTGGTTTGTGGGGAGGTGCTGGGGAGGGGCAGGTGAGGCTGGAAAGGGAGTCCCTCAGTCCCCTCCGTGTCCGATCCTC
AGGGATGGTGTCAGCGGGCCAGGCCGACAGCTCCAGCGAGGACACCTCCAGCTCCAGTGATGAGACAGACGTGGAGGTAATTGCCACCCATCCC
TAGGAGTTGCCCTCTCCCAGCCTCTAACCCTTCCAGGAACCTGTCTGGGGCTTCAGACACCAGTGGGCCCAGATGAGTTCAGCAACTGTGGTAG
GGTGACCAGGAGCTGGAAAGGCTGCAGCCGGAGAGGGGCAGGGAGAGGAGTCTGGTGCTGCTTGCTGCACTCTGAGTGTGAGATCAGCCCTGGG
GGCAGGAGAGGGACACCCTCCTGCCTGTGAGACTAGACAAGCCTTCCTGGGAGAGGTGCCCTGGAGTAGGGCCTCAAAGCACAGTGGGCTTCAC
TGGGAAGGGATGTGGAGGAGGCAGGGCATTCCAGACAGGAAATGGGGCAAGAACAGGGCCCAGAGGCAACAGTGTGGCTGTCGTGGTCTGGCTA
GGGCTGCGGCTGCTGAGAGGGTTCTGGGCCAACTGGTGAATGCCAGGCCTAGACACTTGGCTAGAGTTGGGCAGGACTCTTGGGTTTGTGAACT
TTGACCTGGAAAAGGTCTCCACAGGTTACTTTTTATTGTGTACCTACTAAGTGGCAGACCTGGGGAATATAGTGATGAGCAGGACAGGCAGAGT
TCCTGCTGCCATGGGGCAAGGGAGCGTCCCTCAGATATTTAATTGGTGATGGCTGCTATGAAGTGGAAATGTAGAGGGCCACGCAGGGCAGGAA
CCTGACGTTGATGAGTAGGCGTAAGCAGAAGGAATGGCATGTGCAGAGAAGGCGGCAAAGAGAGTTTGGTGCTTTTAAAGAACATTGATCTTTT
TTTAAGACAGTCTCACTGTGTCACCCAAGCTGGAGTGCAGTAGCGCGATCTTGGCTAGCTGCAACTTCCACCTCCCGGGTTCAAGCGATTGTCG
TGCCTCAGCCTCCCAAGTAGCTGGGATTACAGGTGCACGCCACCACGCCTGGCTAATTTTTGTATTTTTTGTGGAGACAGGGTTTCACCATGTT
GGCCAGGCTAGTCTCGAACTCCTGACCTCAAGTGGTCCATGTGCCTTGGCCTCCCAAAGTGCTGGGATTACAGGCATGAGCCGCCACGCCTGGC
CAAAACCTTGATCTTTAAATGAGAGGGAGAAACCAGGCACGGTGGCTCGCACCTGTAAGTGCAGCACTCTGGGAGGCCAAGGCAGGAGGATCAT
TTGAGGCCAGAAGTTTGAACCCAGCCTGAGCAACATAGTGAAACCCTGTCTCTACAAGATGTTATTTTTTAAAAAAACAAATAAATTATGGGGA
AAGTGACACAAGATGAAGTCCTAGGTAGACAGGGGCCAGATCTTCTGTCTGACCTTGAGGACCACCATAAGGAATTTGGGTTTTATTCTAGGGG
CTGTGAGAAGCCATGCCAGCAAGGAGAGATGGCCTGATTTGCATTTAATAAGATTGCTGGCTGCAGCCTGGGTTGGAGGGCAGTGCAGAATAAA
AGCAGTAAGACCATGTAGGAGACTATTGGAGTAATACTGATACTGGCATAAGAGGCTGGGGGCTGGGAGACCAAGGTGTCCACAGTTGGAAAGA
TGTTTTCAAGGGAAAACCAGCACAAGTCAGGGATGAACTGATCTGGTGCCCAGGGAACTTCTCACTTTACTGTGCTGCCTGGCCCAGTAACCCT
GTGAAGGTTGAAAGCTCACCCCCATCTACCCCAGGCCTTACCAAGGGCTGCCCTCCACCTTCCGTCCTCACCCTTGCGCCCTAGATACCACCAG
CTGTAAGGACATCCCTGGCTTCTGTCACCCCAGGCCTTGCCAAAGGGAAGAAGACCACTCCCAGCCCTCCATAAACACACAACCTCAATGGGCT
GAGGCTGTGCAGAGCTCCAGTAGCAGACGCCCCAGAAACCCAGGGCCGCAAGGGGTCTGATGGATCTCAGAGGTAGAAGTAGGGCACTGACTGC
CTGGGAAATGTCTGAATTGTGGAAGTGTAGGAAAGAGCTCACAGAGGGAAGGAATTTGCCCAAGGTGTGGCAAGCCAGTGGCAGAGGTGGTGTT
CACATCCTGAACTCCTGGCCCTCAGGAGGGCTGCTGTGATCAGATACTTGAGTTTAAGGGGCAGACAGGATGCGCTCTCCTGATTGAGATGCAA
GCCCTTCTCTCAGGCCAAGCCCGGTGTGTGCCAGGTGGGAGATGAACGTAATGGTTTTGAGATCTGTTCCAAAGTCAGACAGTCCAGGGTTCCA
ATGGGGCTCTGTCCCTTCATATTGATCATGAGTGATTTTACCTCTTTAAGCCTTGTGTACTTTTCTGGAAAATGGATCTAAAGCTAATAGCCGT
CTCAGGGAGTTGCTTAAAAATCCAGTGACATTAGGAAAGAGCTTTATCAACTGCTGAAGCCCCTAATACAGAACCTTAGGGGGAAACAGTAATT
ATTATTCATTTTCTAATTCCATCCTCTTGTTCCAGGGGAAACCCTCAGTAAAACCAGCCCAGGTCAAAGCCTCATCAGTTTCTACTAAGGAGTC
TCCAGCAAGAAAGGCGGCCCCAGCCCCTGGGAAGGTGGGGGATGTGACACCCCAGGTCAAAGGAGGGGCCCTGCCCCCAGCCAAGAGGGCCAAG
AAGCCAGAAGAGGAGTCAGAGAGTAGTGAGGAGGGATCTGAAAGTGAGGAGGAGGCCCCTGCAGGGACACGAAGCCAGGTGAGGCCTGGAGGAG
GGCTGCCCCTTGGAGGACCTGCGGGTCCCCCAGCAGCCTGAGCACTCTGCCATGAGCACCTCTGCCACTGGAGTTGGGGAGAGGGAGGACTATG
GTGTGAAGTTGAAGAGCAAGAACAGCCTGCTTCCCACAGGGGAGTCTTGCACTCCACTTTGTCTGTGACCAACTTTACCTCAGTTTCTCTGCCT
AGTAAATGGGCCGCTGTCCTCGTCCCAGCCAGTCCTGCTGTGAGGGACTTGAGAAGTTTTGATTGCCTAATCCTCTGACTTCTCAGGACAAACC
AGACCCTGAGCAGGCAAACACTACGTTAGCTAAAGGGAATCCAGTTCCCTTCCCCATCTGCTGGGAGTGGGCAGGGCTGCAGCCCCAAGGCCCT
GGGTGTATCCCAAGGGCAAGACAGGGGGGCTGGAAAGCAATGGAAGTCAGAGCAGGAAGGAGGAAGAGTGAGGGTCACCCACCCAGCCCCAGGC
CTGGGCAGGACAAGGCGGGGGCATGCAGTAGATAAGCAGATCCCCTGCGTTAGCTGCTGGTAAATGGTAAAGAGGAAGTGGGGGACATAGGAAT
GGCAACTTGAAGCAGAGTGCTCTGGGCTGGCCTTGGGAGAAGGCGTCTCAGTAAAGACCAGAAGGAGGAAGCACTCTTAGGAAGGTCATTCCAG
GCAGGGGCTCAGCAAGTGTCAAGGCCCTGAGGTAGAAGTAGGACTACGAATTGTGACAAATTGTTGTTCCCACCCCATCTACAGGGAACTTCTA
CGCCTCGGCTGTCTATTTTGCAGTGTCTGGGGGCTACACAGTGTTGGATGGGCATCCACTTTGAGAAGGAACACAAATGTGAATTTTTTTTTTG
AGACAGAGTCTTGCTCTGTTGCCCAAGCTGGAGTGCAGTAGCGGCAATCATAGCTCACTGCAGCCTCACTCCTGAGTAGCTAGGAATACAGGCA
CACACCACCACACCCAGCTTGTGTGTGTGTGTGTGTGTGTGTATAGACAGGGTTTCACTGTGTTTCACAAGCTGTGGACTCAAGCAGTCCTCCC
ACCTTGGCCTCCCAAAGTGCCGTGATTACAGATATGAGCCACCGCACCTGGCCATAAATGTGAACTTTTATGTAAAATCTGCTTTCTCCATGTT
GGCTCAAAAATTTAAAAAGGAGCTAGCCAAGCAAGACCTGTGTGGAGCAGCCTGCACTTTCTGCATGAGAAGCTTCTTAGAAGATCAGAGCTGC
TGTTTTCTAGGGTTGTTGGCCCAGGGCTGAACACCCAGGGCCTTCCAGGGAAGACAGGGCCTGCAGCCACCCTGCCAAAGCCCCAAGACAGTGC
AGAGCCCCACCGAGGAGTTAGTGAAGAGGGGGCCAGGTGTGCAAACCTTGCCCCATGAGGCTGAGGGTGGGCTTCCAGGGGTCCTGCTGCTGTC
CATGTGCAGAAAGTCCAGTGAGGGCCTCCCTCAGTCCCTTCCCCACTTTCAGACTGCCTCTTCATTCCTTCCCTTCCCTGGGCCTTTGCACACC
CCCTCCCCACAGCCCAGTCTGATGATGCTCCCGATAAGCCAGTGCCTGTGCCTCCAGAGCTCTCTGGCCAGTGGGAGCCCAGCAGAGGACAGGC
TCTAACTACCCAGTGTGCTGGGGCACCAGGGGGAACCATGTTAAGGAAGTGGTGTCTCTTAGGGAGCCAGAGACAGAGGAGGCAGTGGCTAAGT
GAAATGGAACAGGGGGCATGGAGGCTCCAGGAGTGAGAGTGTGTGAAGCTGATCGATTTTTGAGAAACCAGGAGGCCAGTGTGGCCAAAGTATC
AGTCAAGGTGGGGAGTGGGGAGGGAAGCAGGGGAGGTCTTAGGAGCCTCATTAAGGCCTCTGGACTTTATCCTAAAGGCATCACCAGAGAGTTT
TCACAAGCAGGAGAGCAAGCTGCCCTTTCTTTTTCACCAGGTAAAGGCCTCTGAAAAAATTCTCCAGGTCAGAGCTGCCTCAGCCCCTGCCAAG
GGGACCCCTGGGAAAGGGGCTACCCCAGCACCCCCTGGGAAGGCAGGGGCTGTAGCCTCCCAGACCAAGGCAGGGAAGCCAGAGGAGGACTCAG
AGAGCAGCAGCGAGGAGTCATCTGACAGTGAGGAGGAGACGCCAGCTGCCAAGGCCCTGCTTCAGGTGAGGCCTGAGGAGGGAGACTCCATGCA
GCCAGGCCCGTCCCCAGAAGGCCTTCTCAGGACTTGTTCTCCCACTCTGGGCCAGAGCCCCGGGCGTGCCTCAGACCCCAGCCCCTTACTCCCC
TCTCACTGTGTGGCATCATTTGCCCTATCTGGTCTTCTGTACGTGGTGTCCTGTGTCTCCTCACACGTCCATCCTCTGGGCTGTCTCCCCTTGT
CTTGTTTCTCCAGGCGAAGGCCTCAGGAAAAACCTCTCAGGTCGGAGCTGCCTCAGCCCCTGCCAAGGAGTCCCCCAGGAAAGGAGCTGCCCCA
GCGCCCCCTGGGAAGACAGGGCCTGCAGTTGCCAAGGCCCAGGCGGGGAAGCGGGAGGAGGACTCGCAGAGCAGCAGCGAGGAATCGGACAGTG
AGGAGGAGGCGCCTGCTCAGGTGAGGCAGAGGGGAGGGGTGGAGAGTAGCCCCATGCCTAAACCCCAGCACCTGCATGGGTGTGGCCACCTTTG
CCACATCCAGCTCCTGTCTCCACACGTCCACCCTCTGGGCTCTCCCCTCATCCTGTTTCTCACTCCAGGCGAAGCCTTCAGGGAAGGCCCCCCA
GGTCAGAGCCGCCTCGGCCCCTGCCAAGGAGTCCCCCAGGAAAGGGGCTGCCCCAGCACCTCCTAGGAAAACAGGGCCTGCAGCCGCCCAGGTC
CAGGTGGGGAAGCAGGAGGAGGACTCAAGAAGCAGCAGCGAGGAGTCAGACAGTGACAGAGAGGCACTGGCAGCCATGAATGCAGCTCAGGTGA
GGCTGGAAGCCGCCCTGCATGGCCTGTGCCCTGCCTCAAAAGACCTCCTGTGGTTTTGACTTTTCCTCTCTGAACCTAGAGCCCTGTGCGGCTG
GCTTCGTTTGCTCTCCTCCCCTCACTCACATTCTCCTTCTGGACTCCCTCCCTAATCTTGTCCTTTGTGTCTCCCAGGTGAAGCCCTTGGGGAA
AAGCCCCCAGGTGAAACCTGCCTCTACCATGGGCATGGGGCCCTTGGGGAAAGGCGCCGGCCCAGTGCCACCCGGGAAGGTGGGGCCTGCAACC
CCCTCAGCCCAGGTGGGGAAGTGGGAGGAGGACTCAGAGAGCAGTAGTGAGGAGTCATCAGACAGCAGTGATGGAGAGGTGCCCACAGCTGTGG
CCCCGGCTCAGGTGAGGCCCCTTCCTGTAAGGCTCTTTCTTTTTCCCCCCCACTCAGAGTGGAGCTGCCTGTGGCCTCCCAACCTCACACTGGG
ACTCTGTCTACAATCTTAGTTTCTTAATGTCTGCACACACCTACCCTGGGCTCCCTCTCCCGATCCTGTGTATCTCACTCCAGGAAAAGTCCTT
GGGGAACATCCTCCAGGCCAAACCCACCTCCAGTCCTGCCAAGGGGCCCCCTCAGAAGGCAGGGCCTGTAGCCGTCCAGGTCAAGGCTGAAAAG
CCCATGGACAACTCGGAGAGCAGCGAGGAGTCATCGGACAGTGCGGACAGTGAGGAGGCACCAGCAGCCATGACTGCAGCTCAGGTGAGGCCTG
GGGAAGGAGGCTGCTACATGGCCTGATCTGCCACACCACAGTCAGCACCCCCGGGGAGCTGTGCTCCCTCAGGCAGAGCATGGGTTTTGGCTGG
TGGGGCAGAACAGATGGGGGACTCTGAGTTCAGGAACCTTCTCCAGCCTTTCTTTGGTGTCCCCTCAGGCAAAACCAGCTCTGAAAATTCCTCA
GACCAAGGCCTGCCCAAAGAAAACCAATACCACTGCATCTGCCAAGGTCGCCCCTGTGCGAGTGGGCACCCAAGCCCCCCGGAAAGCAGGAACT
GCGACTTCTCCAGCAGGCTCATCCCCAGCTGTGGCTGGGGGCACCCAGAGACCAGCAGAGGATTCTTCAAGCAGTGAGGAATCAGATAGTGAGG
AAGAGAAGACAGGTCTTGCAGTAACCGTGGGACAGGTGAGGCCTGTGTTTTCTGGGCGGGCCTCAGGGCCGCCCCTACGTGGTCCTTTGGACTC
CTGGAGACACCTCTCTTCCCCTTGTCATCCCAGGCAAAGTCTGTGGGGAAAGGCCTCCAGGTGAAAGCAGCCTCAGTGCCTGTCAAGGGGTCCT
TGGGGCAAGGGACTGCTCCAGTACTCCCTGGGAAGACGGGGCCTACAGTCACCCAGGTGAAAGCTGAAAAGCAGGAAGACTCTGAGAGCAGTGA
GGAGGAATCAGACAGTGAGGAAGCAGCTGCATCTCCAGCACAGGTGAGGCCTAGAAGGAGCAGGCCCATCCCACCCACACCTGTTCCTGAGCCA
GGGCTGAGGATGGGCTTGACTGGGGGCTAGTGTTGCCTGCAGGTGTGCAGAAGCCTCAGAGGTAGCCTCAACACAGCTTCCTTCCCTATCTTTC
ACTCCATGTCTCCTCTGCCCATCAGAGCTCCAGGGTCTCTGTCTTGTTTGCCTTTTGAGGTCCTCCTGCTGATTGATTCCCTGTGGTTTTCACA
AGCTCTGGAGTTCCCACCCTGCCAGGTGCTGCCCTTGTCTGGGGTTGCTGAGGCCCTGTTCCTCAGGGAAGTCCTGTCTCTGTGGAGCATGGAC
ATGCTCCTGCAGTGTGGAGCGATGGGGTTGAGATAGAGGGTGTGAGCCTGGGCTGCGCAGCATGACAGGTGAGAGCATCTAGGCCTTTTACAGG
AGGTGGCAGCATCTGAGCCATGGGCTGAAAGAGTTGGCCAGGTGAAATGGGCAGGGAGAGTGTTCCAGGCAGAGGAAGCAGTCTGTGCGAGGCC
AGGGAATGAGAGAGTGCCTGGCTTGGGTGCCTTGCCACAGGCGGCACATTGTGGTGGGATGGCTGGCAGTTGTTTAGAGCCAGACCGCCTGGGT
TCAAGTTTCAGCTCCACAGCCTGGCAAGTGCTGTAAGCCCTCAGAGCCCTTACTCTCCTATAAACAGGAAATCAGCCCTCACAGGTTTGGGTGA
GAATTTAGGTGAGATCTCACCTGCTACACCCAGCAAGGGAGCTGCACACAGCAAGTTGTCTCTGTCACCCTGACGATGGGCCAGGCTAATGTGG
AACAGATCAGGAGTGAGGTCTCCAAGCCTGGAAGAGCTGAGAGAAGATGGCTCACTCCCTGCACTCACCCACCCACTGTTTATTATTTTTAATT
GTCGCTCTGTTTTTTTCTGTAACCACTCAGAAATTAGTCATTTCTTTTTTCTCTGTACCAGAGCACACATTTCTACTGATTTTTTTTTGTCCTC
CTCTCGTTGACTTGTGGGACCTCAGGGCTCTAGAATTCCTCCATTGAGGTTTTTAAGCTCTGCAGGGGCCCCTTCCAGAGCCGCCTCTACTCTT
TCCCAAGCTGCAGGCACATTCCAGACCCAATGCCTTACAGAGTTTCATTATGCAGCTCACTTAAATATTTGTCTTCCCTTCTGAGCCTCCCACC
TGTACCATGAGGTGGGTGTTTTATTTTTATTTTTTGAACCATGGGCTATTTTGAAGTAAGCCGTTTAATTTCCAAATATCTGGGATTACCCAGG
TTTCTTTTTGTTGATTTCTAATTTAGTCACATTGTGGTTGGAGAACATATTTTGTATGATTTAAATCCTTTTTTGTGTATTGAGACTTGTTAGG
TCAAGTCAGTCTTGTGTCTTGTTTCTTGTGGAAAACATGGCTCCCCTCTGGAGGATTTACGTGCTCTACTGATGAGCACCTCGGCCTGAGTCAT
ACTTGGTCCACACTGTAGGGAATCTTCTTGAAGTCCTGATTCTTCACCTCATTTGACCTCATGTGTTTGAGGAATGCTGGGGGATGGGGTTGTT
TTCTGACACTGACCAGATGCCATCCCTGAGTGAGGCAGGGGCCTTGAGATGCTGCCAGAGTCCTCATTCTGTTTCACATTAGGCTTGCTGTCAC
TCCTGTTAAGGATCTCACTCTGGGAATTTCCCTTTTTTTTTGTTCTCCTTCTATTTGTTGGAATCCCTGACAGGCCTTTTGGGGGTTCTCTGGA
TATCGGTTAAGGCTAGATTAAGCTGAAGAAAACCTTAAACAACAAGTATCTTGTTCTGCCGTGGCTGCTTGAGCTCCAGCTCTGACCTCAGCAT
TCCATCTAGCCTGGAGGAGGAAGAGACATGTTTCTGACCTTTTATGGCACTCAACTTACATCCTATGAGCCAGAATTTAATGACATTGCCACCT
AGCTGAAAGGGAGCCTGGAAAACATCTGTTTTAGGTAGTCTTGTGCTTAGCTAATGCTAAGGAAGGCACGCACAATGAGTTTGAGTGTTTATTA
TGTTCCAGACATCATGTTAAACTCTGAATCTCCTATTTAGTCTTCATCATAACCCGTAGGTGGGCCTTATTATTATCTCCATTTGATAGAGGAC
TGAACTGAGGCCCAGTGAGATCAAGTGATGAGCTCAGGTTCACACGCCTATTGCATGGAGGAGCCAGAATCCAGACTCGGGCTCCAGCTCCAGA
GTCTGTATTCTTGGCTAGCTGCTTTACTCAATCTCACCTTCTCCCTCCTTAATTCCCTTTTCTCCACTCAGGTGAAAACCTCAGTAAAGAAAAC
CCAGGCCAAAGCCAACCCAGCTGCCGCCAGAGCACCTTCAGCAAAAGGGACAATTTCAGCCCCTGGAAAAGTTGTCACTGCAGCTGCTCAAGCC
AAGCAGAGGTCTCCATCCAAGGCAAGTGGGGCCAGAAGCCACAGGAGGTGTGGAGGGTTGGGGTAGAGAGGAGGACCAGTCACTGAGCCCAGCC
AGGAGAAGGTGCGTGCATGGGCAGGCCACCCACCCAGAGTTGTGCCATAGTGGGGAGTGGGACCTGAAAGGAATCACTTTTGCCTCCAATACTA
TTATCCCCCTGCAATTCAGGTGAAGCCACCAGTGAGAAACCCCCAGAACAGTACCGTCTTGGCGAGGGGCCCAGCATCTGTGCCATCTGTGGGG
AAGGCCGTGGCTACAGCAGCTCAGGCCCAGACAGGGCCAGAGGAGGACTCAGGGAGCAGTGAGGAGGAGTCAGACAGTGAGGAGGAGGCGGAGA
CGCTGGCTCAGGTGAGGGGGAGGGAATGGAGATCATCCCCTACATGGGATGTAACACCTTTGCCACATCCAGCTCCTGTCTTCTCACACGTCCA
CCCTCCAGGCTCTCTCCTCTCATCCTGTTTCTCCCTCCAGGTGAAGCCTTCAGGGAAGACCCACCAGATCAGAGCTGCCTTGGCTCCTGCCAAG
GAGTCCCCCAGGAAAGGGGCTGCCCCAACACCTCCTGGGAAGACAGGGCCTTCGGCTGCCCAGGCAGGGAAGCAGGATGACTCAGGGAGCAGCA
GCGAGGAATCAGACAGTGATGGGGAGGCACCGGCAGCTGTGACCTCTGCCCAGGTAAGACTTGCCAGGCCTCTGAGCCACCAACACTCACTCCT
CAGAACGGGGGTATAGGGCTGGGCGTGGTGGCTCACACCTGTAATCCTAGCACTTTGGGAGGCCGAGGCAGATGTATCACTTGAGGTCAGGGGT
TCAAGACCAGCCTGGCCAACATGGTGAAACCCCGTCTCTACTAAAAATATAAAAATTAGCTGGGCATGGTGGCGGGTGCCTGTAATCCCAGCTA
CTCGGGAGGCTGAGGCAGGAGAATCCCTAGAACCTGGGAGGCGGAGGCTGCAGCAAGCCAAGATCATGCCACTGCACTCCAGCCTGGGTGATGA
GAGTGAGACTGTCTCAAAAAAAAAAAAAAAGAAAAAAAAGAAAAGTGAAAAACCAGGGTTTAGGGCCAGGTAAGGAAAAGAAAGGAAGGCCAGG
TTAAGGCTTAAGGCTCCAGCTCCACCATGCCCTCCTCCGTCTGCTGTCATCCACATCTCCTTAGGTAAAACCTGCTACTAAAACCCCCCAAACG
AAGACTTCCGTAGCTAAAAGTGTCCTGGGTCTATATCTAGGTCGAGGTCTCCACCAGCTCTATAAGCTAGGCCAGTATTTCCCACCAAGGCATA
TGAGACCAAGAATCTACATGTTAGTTATTGTGCATACGTTAACGTTATACAAAATGACTCTCCCTGTCAGAGTCCCAGGGAAGCTTGGAGTGTG
GCACAGGTCCCCCTGAGGTCCTGGAGAGATGGAGGAAGTCTCATCCTGCACCTTGGCCCGGAGCCTCACATAGGCAGGCGCATCTTCTCAGGCA
GGGCTTGTCTAGCTTAGTGCAAAGCCTGGACCCTGGAGAGTGGGGAGAAAGAAAATCTGAACAATTCCCTCTGTCTGCTTGCAGAAGATAGCAG
GGCTTGCTACATCCTACAGAAGTGTAGGATGACAAACTCCAGCACTGCCAAGGCACAGGTGTGTGATGGAAATGACTGATGCAGGCCTGGCAGG
AATGATAGATGTCGGCCGGGTGCGGTGGCTCAAGCCTGTAATCCCAGCACTTTGGGAGGCCAAGGCGGGCGGATCATGAGGTCAGGAGTTCGAG
ACCAGCCTGGCCAACATGGTGAAACCCTGTCTCTACTAAAAAAATACAAAAATTAGCCAGGCGTGATGGCAGGCGTCTGTAGTCCCAGCTACTC
GGGAGGCTGAGGCAGGAGAATCGCTTGAACCCGGGAAGCAGAGGTTGCAGTGAGCCAAGATCATGCCATTGCTTTGCACTCCAGTCTGGGCAAC
AGAGTGAGACTCTGTCACAAAAATAAAAAAAAGAGATGATAAATGTCAGGAGAGATGGTAGATAGTGGTAGCAAGTAGGGCAGACCGGAGAGCC
CTTGCCCCATCCAGAGGGGCAGTTCTTATCAGCTTCAGCCAGTCATGACTGTGGAGATGGACCTTCTGTGTGGCCAGGTCTTCCATTTCTTAAG
GGAAGCTGGAAGTCCCTGTGCCAGGCCAAGCTCCATAACCTCTTAGATTTGTTTTCTTCATGAGTAAAACAAGCACTGAGATGAATGCTCCCTA
GCCCTAACCTCTGCCTTTGATACACCAAACCAACAATTACTGGGGGTGAAAGACATTGGCCCCACCCTCAAGGAGCTCACAGTCTGTGGGGCAA
GACAGGTGACCGGATTATCGCAGACTATGCTAGTGGTGCTGACAGCTGAAAGAGCAGGTCTCTGAGAGGCCAGAAAAGGAGGAGATGATTGAGC
TGAACCTGAAGGATGACCAGGCACAAAAGTGGAGAAGGCCCCTCAGTGTGTGCAGAGGGGCTGGGCATGAAGGCACATGGTCACTGAAGGCAGC
TGTCCTGCAGGCCGTAGGCCCTCCCAGGTGCTTGTTACTGGTAGATTTATGGAGGACCTAAATGCCAGACTAAGACATAGGCCCCGGCCATATT
TTGGGCTGTGTAGTTTTTTTGTTTTTAAGTTTTCAATGAGTTCTTAGTCAGAATGGCCTAATCTGTAGGAGAAAGCCCCATTCAAAAGAGCTTA
AATAAGGGGATTGACTGTCTTAAAGTGCACAAAGTCCCAGGTTCCAGCTGCTGAAAAGCTGGTTCCTTTAGCTGAGCGTCAGTGGTAACCAGAT
TTCTTCATCTCTGCACTCCACTAGCTTTATGGTCATAGCAGGGCTGCAGCATCTCCTAAAGTGCTGGGGGCAGGCCAGGCACGGTGGCTCACAC
CTGTAATCCCAGCACTTTGGGAGGCTGAGACAGGCGGATCACTTGAGGTCAGGAGTTCAAGACCAGCCTGGCCAACATGGCGAAACCCCATCTC
TACTAAAAATACAAAAACTAGCTGGGCCTGGTGGCACCAGCCTATAATCCCAGTTGAGGCAAGAGAATCACTTGAACCTGTGGAGGAGGAGGTT
GCAGCCTGGGCAACAGAGTGAGCAGAGCAAGACTCCATCTCAAAAAAAAAAGAGATTAGGGGGTAGACTTAAGGCTACTCAGACCTCAGGCTGG
ACGTTCTACCGCCTTAGCCATAGACAAGAGGCCGGTAGTTCCCATGACTGGCTGGGAGGAGGGCCCACCCTCCCTGAAGCATGTGGTAGGGGCC
ACTGAACAGAGTGAGGGTTCTATTAGCAAGGAACAGATGGGGGCATGGCTGTTAGGAGGCAGCCTGCAGTGTGCATTGTAGTTGCCCCATTTAA
AAATTGGAAATGACATGTACAAATCTTCCAGCTTCTCTTGATAAATTGGAAGCTCAGGCAGCCTGGGTCCTACTTTTTTGGCTAGGCTGAGTAG
GGAAAAGGGGGGCCCCCATTCCAATAGGGACCACTGAGCTGCCATCACCACCAGCATTTCCACCCAGATCTCTGAGTCCTCCTCTGACTGCAGT
GCTTGCTCATCCTTCCAGCCCCCATCATCACACCTGCCCCAACAGGAGGGGATGGTAGCCAGTGGAACACTTCTAGGTGGCTCCTGCTTTTGGC
TGGGGGTGTTACAACCGGAGGTGAGCTGTGCTGGGACCACTGGCTTGTGCCTGCAACCTTGTCCCAGGCTGCTGTCACCTCTTGTCGAGACATT
GGGCCAACAGCCCTGCAGCCCTCAGGTTTTAGCTGCATCGGAGTCATAAATCCCTTTCATGTTCGGAGTCAGCTCTGGAAGGAGCTAGCAGGGT
GGAAGCATGTTTCCAGTGGCTGTGGGGCAGGTCAGGCCAGCAGGTATCCCTGTGCATGTGCAGGAAGGGGCCACGCTGCCTCCCCTGGAAACCA
GAGTGCCTGAGGGTCATTGCCTACTGGCTGTTTCCCCCTCAGCAAATGCCCCACTCCCCGACCACGTGCTTATCCAGGTCTTGTCCCCTCAGAA
GGACAGTAACTCCAAACCTGCCAGAAGCAAGACCCTGGCCCCAGCACCCCCAGAGAGGAACACGGAGGGGTCCTCGGAGAGCAGTGAGGAAGAG
CTGCCACTGACCCAGGTGCGCCATGCCTTCTCTCTGCAGATGCTGCAGGATCAGAGCTGGGCTGGCAGGCCTTGCAGGAGAGGGCAGATCTTGC
CCATAGGGAAAATCTCGCCATATTTAAACAGCACCTCACAGCTTCCAGAGCTCTTTCCTCTATTGTGACATTCATCTGAGCCCTGTGAGGCAGG
CAGCAGTACACAGTTAAACATACAGGCTTCAGAGCCAGGTGGTGTTGGGGCGAAGCCCAGCTCTTCCACCCAACAGCCCCATGGTCCTGGGCAG
ATGCCTTTTCCCCTGTGTGAGGCTCAATGTTTCAGCCATAAAATGGGGACAGTACTGGCACCTCTCTCCTGCCTCTGTGCACTGGTGCCAGCAC
ACAAGGTACTTGCCACAGTCCCCTGCCTAAGAAAGGGTGCCTGTTAACTGTGAGAAGGGCAGGGAGTGTTGCCCCATTTGACAGATTGGGAATC
AAGCCTTAGAGTTGAAGCAGTTTCCCCAAATTCTCATGATTCATAAGAGGCTGAGGAAAGGTCCAAACGCTGGTCCCCTGGCTCCCTGTATCTC
TCTGTTTTCTGTTTAGGACCAGGAGTCTTCTTGAATCCCATGAGCTCTGCCCAGGACCCTCTCTCAAGCACCTGTGGCATACCTTCATGTGCTC
CACTGCACCCAGAGCTTAAGCCACAGTCCTGCTCAGGCTACACCTCCAAGGGTGGGACCAGGCCCACCTTATCTTCCTGTACTCCAGAGGCCCA
AGTCAGCCATGCTGGTGTGGTCCAAGCTTCTGTGTGAACCCTGGCCCTTCCATCAGACAGCATTACCCTTTGTCCTCCTCTAGCCCCAAGCTCC
CAGAAGCTTCTGCCAGGCACCTCAGAGCACCAGCTCCCCTGTCCCAGGGCCTAACCACAGATCCTGGAGTTCTGCAACCTCCAGACCAGAGCGG
TCTCATGAACCACCTCCCCTTGAATATCACATACCACATTGTTATCAATTCATCAGTAAGGTCAGATGCTGTTTGCACACCTCCAGCAATAGGG
TGCTCACTGCCTCTCACTTTCCAATGGCACAGATTACAAAAGTAAAGAACAACCACCACCAACATCGGTAACTCCCTGTATTCTCATTTTTCTG
TTTAATCTCCACAACAGCCCTATGAGGGTGGCTCCCTTTGTTGTTTGCATTTTAAGGAACTGGGAGTGAGGCTCAGAGAAGTTGAGGGACCTGT
CCCAGCATCACATGGCTGATGACAAGTGGACAGACCTGGTGTTTGAGCACAGGCCATTCACATTCTGACCCGTTGCCTGGTGCTGCCCCGACAC
TCTCCTCTCCTGGGCTTGGGCATTGCCCTCTGGCTCTGTGTGGAGCCTTTACGAGGCCACCTGCCTCTCTGAGGGATCTAAACAACTGGGAGGG
AGGGGACCGCTCCCCCTTTCTCTGTGGGGGTGGACTAACACAATTAAAATGAACATCCTGCTTAAACTGAACCATTTAATTGGTATGTTAGTCC
TGAAGATTCTGGAGTCTCTATTTAAAAAGCAGAAAAGAAGTCAGCGGTTTGTACATTCCCCCAGGAGGATGCGGGGAAGACGGGCATAGCCCGG
GGAAGCTGCAGCCTCTTCCTGATTGAGAGATTTTTAGTTCCTAATCCCCAGCTCACTAATTATGATTATTTTCATTATTGTACATTTATTGAGC
ACCTCTCTATTGACAAGTTTCAGTCTGATTTTATTAGCCCATCAGTGGGGAAAGGGATTGAGGCCAGGGTGGTGGGCAGAGTCCCAGGCTGGGA
GGCAGGGATAGATACCTTGGCTCCATTCCTAGACCCCTCACTGTGCCTCCGCCCTGTTGTGCAGAGTAGACCTCAGGCCAGAGTCTGTGCTGGG
GTCAGGACAGCTTCCAGATTTAAAAACAAGTAGGTGATACATATGTTAAATAACTTAATTTAGCTATTCCACAATGTATATGTGTATCAAAACA
TCATGTTATATACCACAAATATATGCAGTTTCACTTGTCGGTAATATTTTTTAAGGCCAACTTCTTCTATTAGTTTGTCAAAGACGTAGGCCCT
GCTCTCAAAGTTTTAGGTTGAATCAAAGAAAAAGGAAACCTGTAAGAAACTAAATTCTGGTGTTACATTGAGGCCTAGCATAATTGGTTCTAGA
AGGAGCTCCAGTCTGGTGCCTGGAGAACTAACTTCTAATCCTGGCTCTGGCACCAGTTTGCTGGTTGCCCTGGGTGAGGCCACTTGCCTTCTCT
GAGCCTCATTCATCTCAATCTCTGGGATGAGACTTCCTGGGTTGGTGGGGTGGACCTTGAGGCTCCAGGCCACATCCCCAGAGAGTAGAATTTC
TCAGTCCCCTGGAGTGGGGCTGGCCTAGATCTGTTAAGCAGCCTCAGGCAATTCTGAGAGAGGTTCTTGGAGTGTTTGTTGCCTTTGTGGTCAT
GTCCCTTGCCAGGGTCCGCAGCCTAGGCCATCAGCCCCCTCTCCCCACTCTGTGACTTGCTGGAATGAAGGCACAGTTAGAATGAGCTCAGCTT
TGACAGGATGGGGTGAGCACTGTCCAGAAAGCCCCTAGGTTTGGTTCTGCAGGCATCAGACCTACAGGTAGGAGGGGTATCCCGGGGTTCCCTC
TGGGTCCTTACTGCCCAGCACCTATAGGGAGGTACAACCAAATGTTCAGGCCAGTGCTGGTTTTGCTGCTTCACTCCCATCAGTTTTTTTTCTA
ACTGACCCCAGAACTACATTCCTGTGTATACTGTGCTTTCCAGTTTCCACAGTGCATTGGAACCCCTCCAGTGAATGAGCCCAGGCTGGGATAA
CTACTAGCCGTTTCATTAAACGGGAAACTGAGGATCAGAAAGTTGAGGGGCTTGACCACACAGTGAAGTCATGAGAGAGGCGTAATGAGAAGCA
GGTCTTCTCCTATACCCTGGGCAAGCTTCACTGCCCACCTTTATGAACTCTACAGGCTAATGCCCCGCCCCAACCCCCCACAGCCAAGCCCCCT
GCACCACCTCCCTCCTATGGCCCAGAGGCCCAGTGGGGTGGCGTGGGCAGGTGGAACTAACAGGAGGCTTTTGCTGCTGAGCTGCTTTTAACAT
CCCAAGACGCTATTGTTTGCCTCAGTGGGTTCGTTGCTTCTCTTCGTCCCCGACAATCTGTTCCTGTCTTCACATATATATAGATCCCCCCTCC
CTCAACTGTAAGCCTTCTCCCACTTTCCATCCTTCTCTTTTTTATATCGTGGGAAGGAAGGCTCTGGAAGTGGCAGATCGGAGCAGCCGCGGGG
GAGGCTATTTGGCAGGGGTAGGAAAGGCCTAAGCCCAGAGAAGCTTCCACCCTCAGGCAGGCACCTTTCTGTTTTACTTTTTACTTTATGCCTC
TCCACAGCACCTTAGCACCCTGAGGAGCCAGGTGTGCTAAGTCAGGCTCAGGGGGTAAACAGGATGGTGAAACAGAGACCAGTCTGGACCTGAT
TCCACTCCTCTCTTAGCCTCAGGCAGCTCCCTCTTCCTGCCCTCCAGCTCTTCACTGGCCAGAGAGGGAGCAGAGCTCAAACCAGATCTGACAT
TTGGGGAATGGAAAGTAGACCTGACCAGCTCCCTGGCTCACAACCATGTGTGTTCTCGTTAGCTGGAGGTTCAGGAAGGAAATGGGCCTGGAGA
TGCCCTCGTGTGCGGTGAACATGTTCAGGGATGGTGCAGTTCAAAACCAGACTGGCCTGCAGAATACCAGGGAGCTGCTGTTGCTTGTTTGCTT
TTTCTTTTATTGTTTCCTTGGAAAATTTAAAGCATACCCAAAGATAGATAATGTAAAGAACCCCCACGTACCATCCCCAGCCTCAACAGTTAGG
AACTCCTGAGCAGGCTCGTTTCACGCAAACCCTCCACTCAGTTCCCCAACGCCCCTGGATTACTTTGCAGCCAGTCTCAGACATGTCAGCTCAT
CCACCAGGATTTCAGTATGCAGCTCTCAGTTATAAGGACCTTTTTAAGCACAACTACGATACAGTGTCACACCTAATAAGTTGATCTACCCAGT
CAGTGTCCCTATTTCCCTGATTGTCTCATAGAGTTCAGTTAAAGGCCACGTACGGCATTGAGTTATTAATCCTCTAAGTCTTAATCTGTTGGTT
CTCACCCACTCACCCAACCCTTGTTTCTCTTGTGCATTTTTTTGTTGAAGAAACTGAATTGGTATAGCTGGGGAGTTTCATCCTGGGCCATGTA
TCAGGAGGGCTGCATCAGAACCTCTGGGGTTGGGGCAACTAGACTGTAAATACCTCCCCAGGTGAATTGCATATTCCAACCAGGCAGCCTTGGA
AAACTGCTTAGACCACTCCCTGCATTCAGCAAGTAACTGTCTCTGAAGCCAGCCGGGGAAAGAAGGCAGAATTGGACATTCTGGCCGTGGCTTT
GCTTCCAGTAGGGACAGCAGCCTTGGGTCGCTCCCTTTCCCTCTGGGCTTGTGGTTTTCCCACTTTGTATAAGGGAGCTGATCCTGCTGAATGT
AGGGCTTCACTGTGAGGGTCAGGTGGGCTCTGGGAGTTGTGAAGTGCTGTGCTGGGTCTTAGCAGATGTGTGGGGTGGGAGGGGGCACCCTGGG
CAGCTGGAATGGCTTCTGTGCCCAGCTGCCCCTGAGCTCAGTGGACCCTTTGCCTTGTAAAACACTCCCTAACCCCATCACCTCCTTTCCCTGG
CCAAGCCTTTAATCACTGGGGGGGTGTTTTGTTTTTGTTTTTCAAGGTGATTAAACCCCCTCTGATTTTTGTCGACCCTAATCGTAGTCCAGCT
GGCCCAGCTGCTACACCCGCACAAGCCCAGGCTGCAAGCACCCCGAGGAAGGCCCGAGCCTCGGAGAGCACAGCCAGGAGCTCCTCCTCCGAGA
GCGAGGATGAGGACGTGATCCCCGCTACACAGTGCTTGACTCCTGGTGAGCGCAGCCCTTATGCAGTGGTGGGAGGGGCTGCCAGCACTGCCCC
ACATTGAGGCCCAGAAGGGACAGGACACTGGCTGAGTCACATAGCAAGGTGTTGGTCGGGAGGGGCTTGGGGTCAGGTCTCTGGGCCCTGCATT
AGCCATTCTGATTAGGGCCTGGCTCAGACCTGCAGTTCTGGGGGAATAGTTGCCAGAGCTTTTTTGTGGCAGCCCCAGTCATGGAGCCCAGACC
AGTGGCTAGAAGCTGCAGGGACCAGGTTCCTACATAGTCTAAGAAAGAATTCTGTATCAGTTGACTGCCTGTGGAACAAGTGGTCAGGGTGGTA
GTGAGCTTCCCGTCCTTGGTGGCATGTAAAGTAGGTTAGGCAATCCCTAAGCCAGAGACATCACAGGGATAGATTTCATCTATCAGAGAGACAG
GACCAATCTGAGGTTTCTTTGACTTCTGAGAATCTGTGATTCTACCAATGACAACAGGAAAAGGGTTAATGTTACTATTACAGTTATTGTAACA
GTTTTACAAGCAATAATACCTGATCCTGATAGGACAATTTAAAAATGCAGACGGGTGCAGTGGCTCACGCCTATAATCCTAGCTCTTTGGGAGG
CTGAGGCAGGCAGATCACCTGAGATCAGGAGTTCAAGACCAGCCTGGGTAACATGGCGAAACCCCGTCTCTACTAAAAATACAAAAAATTAGCC
TGGCGGGCGCCTGTAATCCCAGGTACTTGAGAGGGTAAGCCACGAGAATCGCTTGAACCCAGGAGGCAGAAGTTGCAGTGATCTGAGATGTGCC
ACAGCACTCCAGCCTGGGCGACAGTGAGACTGTCTCAAAAAAACAGAAAGCAGGCAAAATAGAGATTACTGCTGGGCATGGTGGTTCGCACCTG
TAATCCCAGCACTTTAGAAGGCCAAGGTAGGAAGATCGCTTGAGCCCAGGAGTTCAAGACCAGCCTGGGCAACATAGTGAGAGCCCTATCTCTA
CCTATCTACCTACCTACATACATATGTACATATACATATACTTATACATATATGTGTGTATGTGTGTGTATATTTTTTTAATAAAAATACAGAT
TAGCACATAAGAAACTATATCACCTGTAATTCTGCCCCAAATAATTACTGTTAACATTTGACAAATGCTAACTTTATAAAAACCTAAAATATAC
GTGTGTTTTTAAAGCAAGACCATACTGTATCTAATGTTCAGTAACCTTCACTGGATATTTTATGACATCGGGTTTTTTTCATCTTTACATATTT
GCAGCACAATTCCTAATGGCTACAGAGAAATCCCTTGGGTGGACATGCTACAGTGTGTGTAGCCAGCACCATGTTGGAGGAAGCAGAGGGTTTT
GTTTCAGGGTGGAGGGGTGAGGGAGTTGGCTGTTATAAACAGTGCTTTGAAGAACATGCATGTAACCAGGCCTCTGCAGATAACCACGGGGACT
CTTAGGAGAGACGATTTTAACTGCAAGGAGAGTCGTTTGGTGCCGGTGCTCTGGCCCATTGCTCAGGAGTGGCAGCCCGACCAGTTGGGAGGGG
AGGGGATGAGGAAGCCTGGGGTCACCCCCAGAGCAGCCATTTGAGCTCCCTGAGCCTTGTCAAATGACATAATGACATCTCCTGCCTGGGAGGC
TTGGAGAGGGAAGTAAACAAGCTTCTACCTCTGTAAGCCCTGAGCACAGCTCTAGATCACCAGCACAGGCCGGTAAATTGGGTTATTGCCGCTG
CTGAGGAGGCGATGGGGCTTTTGTGCCCTGATGTGCCCCCATCTCGCTCCATTTCAGGCATCAGAACCAATGTGGTGACCATGCCCACTGCCCA
CCCAAGAATAGCCCCCAAAGCCAGCATGGCTGGGGCCAGCAGCAGCAAGGAGTCCAGTCGGATATCAGATGGCAAGAAACAGGAGGGACCAGCC
ACTCAGGTACCTGGTGGGCAAGGGAGGGTAATGCAGGCCAGTGGGGTGGGGCCCTACTTCCATACTTACCCACATGTGCTGATGGGATGGCCTG
CAATTGCTGTCACGCCCACACTCCAGAGGTCGTGGCCTCACAGCCAGAGGCTTTCTGGCCTCCTAGCTACAACCCCCCAAATCACCTTTGAGTT
ATGAGGAAGAGTAATCTGGGGACACAGCCTCCTGGCCAAGCAAGCCCTGCAGCACTCGAGTCCCTCATGAGATTTTCCAGCGTAGTGGGGCTTT
TGATTTTTTTTCTTCCAGTTTTGAAATGCCATGAACAAAAGTTGCCCTTTTTTGGTGTTTGTAATAGTGACTTCAGCATTCTTCCAAGCCTTTA
CCATCTTCAGTGCATTTTTATTTGGTCCTTGCCTGAATCTAAAAGCTGGGCATTTTAGGGTTAATGGCTGGTGCATTGTTTAGATCATAAAACT
TAGCACACATCCCCACCCCAAGGGCTGCGATGTGACCAGCATTCAGCAAGGCCGGAACCCGGGTCTTCTGAGACCCAGTTCATTGCTCTTTGGG
CCATATCAGTCTGGTTTCCCATCCCACTGAGAGGATGAAGTCCATGACACACTCTGACCTCTCTTCCTGAGGGGTGGGCTAAGGGGGTGTTGGG
GGGCGGTGGGGCTGGTGTGCCACCCACTCCCTGGGCTCATCACAAGCTCCAGGTCAAAAACTTCTGGGAACATGGGTGTCGGATGGGAAGTCAG
GCTGTGGTCACTGCATCCATGAGCATCGGTTCCTGCCAGGCCCTAGGGATACTGCAGGAACCAAGACAGGGACAAGGAATTCCCAGGCCAGTCA
GGGAGTGGTGAGGGGCAGCGATTAAACAAGGAAACCCACATATAAATAAAAAGTATTCACCAGCTGGTAGGAGAGGAAACAATAAAAACTAGAG
AATAGTGGGGTGACCTGCTATATGAAGGCCTCCTGAGACAGCGATGCTTAAGCAGAAAAAGCAGCCAGTCACAGGATGAGCTGGGCCACCCTCC
AGGCAGGGAAACTGCAGGTACCAGGTCTTCGGGGGAAGCGCTTGGGAATGAGGGAAACTGAGAGGTGGGGACAGCTGGATGAGGAGCTGGGGGA
GAGGAGATGAGCCAGGCCGTGGCAAGAGCCACGCCACATTTGAAGGCTCTCAGCAAGGGGTCATGCCCAATTTCCATGCCCACCACTCTCCCTG
ACTGCTAGTGGTTATAAGAAGTAGAGATGGGGGTCCACCAGGTTGGAGATTATCCCCAGTGAAGGTGCAGATTGAGTAGTGGGGCCTAAGCTGC
TTCCCTGCCCTGGGCTGGGAACTGTCCCTTGCTCCTTGAGAACAATGTGATTCGCTCATAACCTGAAGCCTGCCACCAGTTTTGCCCCTTTGAC
TGCCCCTCAGTCCCTGCTCCAGCCCTCACCCCAGCCAGACAGCATCTGACCAGGGTGTGGCACAGCTGGCATCCCAAGGACTTGTGAGTCTGAG
GGCTACCTCTTGCCACCCACAGGTGTCAAAGAAGAACCCAGCTTCCCTCCCACTGACCCAGGCTGCCCTGAAGGTCCTCGCCCAGAAAGCCAGT
GAGGCTCAGCCTCCTGTTGCCAGGACCCAGCCTTCAAGTGGGGTGAGCTTGGGGAGCCAGGGGAAGCAAGCCCACGGAGCGTAGAAGGTGCAGG
CGTGGCCCTGCATTGCGGCACCAGCACTCATCTGCCCCATGACCTCTGCACTTGGTTTGCCTCCCTCGGCCTCAGTGGCCTAATCTGTAAGAAG
GAATTACAGTACCTTTCGTAGTTGGAAGGATCAAATGAGGCTGCATGTGTGCCCCATCTAACACAGTTCCTGGCCCTACTGAAGTGTTCAGGAA
GGACCAGGTCTTACTTGCCCTAATTTTTCCTTCCATTCCTTCTCCTTTCACCGAATTAGGTTGACAGTGCTGTGGGAACACTCCCTGCAACAAG
TCCCCAGAGCACCTCCGTCCAGGCCAAAGGGACCAACAAGCTCAGAAAACCTAAGCTTCCTGAGGTCCAGCAGGCCACCAAAGCCCCTGAGAGC
TCAGATGACAGTGAGGACAGCAGCGACAGTTCTTCAGGGAGTGAGGAAGATGGTGAAGGGCCCCAGGGGGCCAAGTCAGCCCACACGCTGGGTG
AGGGTGCCAGGGGAAAGGCAAGGGTGGGCCAGGAAGAGGGTGTTGTGTGGCCTGGTGGAGCCATAGCTCTGGCCTCAGCTCCATATCTCAGACT
CATTCTGCACCTGTGGCTGAGCCTGGGCCTCAGTTTCCCCACCTGTACAACTTCATGAGGAAGTCAATCCAAATGATTTCAGAGACCTGACTTT
GCTGTTTGACCACTCTCAGCTTTTTGGTATCAGACTCCCTTCACTGGCTCCCAAAAACTCCAGGGCCATGTTTCTGGAACAGTGGAAAGCAGGG
AAATAGAAATGGGGCCTCAGGAATTAGAAATAAGGCTTTGGCATTCAAATGTCGCACCTAGCATGCTGTGACTAGCGATAAGTGTGCAAGGAGT
GTTGAAGCAGTAGGAAGACTTGTGGTGAGGCGGGGCAGGGGATGGGGGTGAGGGACCTGCAGAGAGACCAGGGCCTTCCTGAAGGGCTCTGCCC
TTCCCGGCTGGCAGGGGCCACCTGGGGCTACCAACAGGATACTGTGCTTCTCCAGTAGGTCCCACCCCCTCCAGGACAGAGACCCTGGTGGAGG
AGACCGCAGCAGAGTCCAGCGAGGATGATGTGGTGGCGCCATCCCAGGTAACTGCAAGGGAGAGGACTGGCAGCCCATAGGCCTTAGGGTGGAG
CCCCAGGCCAGGCTCCTGTCTACCCGATCCCTCAGGTCAGGGGTCTGGGTCCCCTCTCTTCACAGAGGCCTTGGGCAAGGTCCTGTCTGCAAGG
CACCACGTGTGGCCCAGCCAGCAGACCACAGCCAGGCTGCCTCCGTCCCCTCATAGAGCCCTGAGTAGCTGACCCTTGGGGTCTGTAGAAGGGA
TTGACCTTGATTCTGGGTGATGTCCGGACAAAAACATTCTTTAGCAGATTTATTTTAAGGTTACTATCTGTATGTGGCAAAAGCTAGTAATTTT
CCATTTGTATTAATAAGAAATTTCCTTTTTAAATGTGCCTGTCTAGAAAGTGTATTAATTAAAATAGTTCAGGAGGCACACGCCAAGGGCTGAA
GTGCTGCTCTGTGCCTTGTTGTCCACCCACTCTGCACTGATAGGGCAGGGTGATCCTAGAGGCCCCAGGCTGCTTTCCTCACAGCAGGCCATGA
CTCGGGCTGGGTTATGGCAGTGGGGTGGGGTGGTGGCAGCCTCTTTCACAATGGGCTTCTTCAGTCTCTCCTCTCAGGTTATATGACCCCTGGA
CTAACCCCAGCCAATTCCCAGGCCTCAAAAGCCACTCCCAAGCTAGACTCCAGCCCCTCAGTTTCCTCTACTCTGGCCGCCAAAGATGACCCAG
ATGGCAAGCAGGAGGCAAAGCCCCAACAGGCAGCAGGCATGTTGTCCCCTAAAACAGGTAAGTTAAGGTCTGCAGGAGGGACATAGCAGGACAC
AGAGGGACAGGGCAGTGGGCCCCTTCTTGCCCTCCTGTAGCAACAGTCCTCCCAACATGGTCCTGTCTGCCCCCAGAGCCTCTCCAAGTGAGAC
CTTGTGGCCTTGCATTGGACCTGAAGATGCTCATCAAAGTGCAGGTGGGGCCAGGTGCAGTGGCTCATGCCCATAATCCCAGCACTTTGGGAGG
CCGAGGTGGGCAGATTGCTCAAGCCCAGGAGTTCAAGGCCAGCCTGGGCAACACAGAGAGACCCCATCTCTAAAATTTATTTTAATTAGCCGGT
GTGATGGTGCACGCTTGTAGTCCCAATTACTTGAGAGGCCAAGGCAGAAGAATGACTTGAAGCCAGGAGGTTGAGGCTATAGTGAGCCATGATC
ATGCCACTACACTCCAGCCAGGGTGGTACAGTGAGACCCTGTCTCAAAAAAAAAGTACAGGTGGGCCCCACGCCCAGGTGTCTGGCTCCCATGG
CCCTGGACAGCCTGGAAGTGGGGGTGTATCCAGCCCTGGGTGGTGCTGATGCACCTGGTAGGATAGCCCTTGCCTCAGAGGAAGCTGCGTCTTT
TGGCTTGTGGAGCAAGTGTTGGCTCTCTCCAACCATCACCATCAACTTTTCTTCCCTGTAAATAGGCTCACGGTGTGGGCCTTGGGCCTCAGAG
GATAGCTTAGACAATAGGCTGACAGTCACGTTTGGAGGACACTCAGCACATGCAGGAGACCCCAGGCAGTAGACTGGCCCCAGGCTGTTCTGAC
TCATTGTAAAGCCTTGGAAATCCCCTGGCCACTCTGGACCTCTGTGTCTTAACCATACAAAACAGCAGTTGATGTATCCAGTCTGTGTCAAGGC
ATGGCACATGTAGAAAATAGTCACATTGTATTTGGAGGGCAGACTGGATTATTCAATTCACCTAACCTGGGGCATCCACACTCCCTGACCCTAC
TGGAGCTAAGGGTCAAATATGTACCAGCTGGGCACACTTGGGACAGCTCCAGGGCTTCCCCAGCCTTGGTGTTCTGGGTGTTTGCAGATAAGCA
GCAGAAAAGTGGGCCTGGCGCAGTGGCTCGTGCCTATAATCCCAGCACTTTGGGAGGCCAGGGCGAGTGGCTCACTTGAGGTCAGGAGTTCAAG
ACCAGCCTGGCCAACATGGTGAAACCTTGTCTCTACTAAAAATACAAAAAACTAGCCAGCATGATGGCAGGCGCCTGTACTCCCAGCAACTCAG
GAGGCTGAGGCAGGAGAATCGCTTGAACCTGGGAGGTAGAGGTTGCAGTGAGCCGAGATTGCACCACCGCACTCCAGCCTGGGCAACAGAGCAA
AACTCTGTCTCAAAAAAAAAAAAAAAAAAAAAAAAGGCAGCAGCAGAAACGTAGGCTCTGGGGAGAGAGGTTTCCGGGGCTAACTGCCCTCCCC
AGCTTCCCTCTCCATTGGCTCCTGCCATCTTGAAGAATCCCCAGAGGATGGAATTGGGCCTCCAGCCGTCCCGGGGGCCCCTCTGCCTAACCTG
GCCTGCCCTGCCCAGTCCACCTCCCCCAGCCCACCGCAGCTCTAGAGCCAAAAGTCGCCAGAAATCCTGGTTGGGGCTGAGGGGCCAAGCTCAC
TGCATGCTGCAGGGGGCAGCCCTAGCCTCAGTGTGACATGAGTACCACATAGAGTGTGACATGGAGTATCCACCTGGGCCTTCCTCCAGGCAAA
CACCTCCAGGACCAAACCACCAAGCCACCTTTCAGAGCCCAGCATGCAGGAGTGTTGTGCCCATGAGACCCAGAATGGGGCAGAGACCTCCAAG
TCACTTGGAAGTTAGAGCCGGTGAGGAGACCCAGTCATGGGGCTTTCAGCTGCAGCTTGCCCCAGGCTGGGTCGGAAGAGGAATTGCTGCTCAC
TTTCTTCCCCAACATTCTCTGTGAGAGGCGGTGCGACAAGGAGACATTTTAGAGGGGGCTGTGGAAGGTTTTTTTTTTTTTACACGTGTAGTCA
GCACACAATTATCAAAGGCAAGCAGAGGCGTGGAAAATTCTAAACAAAGGCTGCACCAGAAGTCTCTCTGGCAGGCAGTAAACCTGCTTAGTCA
GCCACCACGTGTATATACACTGCACCCAGAGCTCCCCACCTTGCCTGGACCCCCTGACCAGGAAGGGCCGAGTGTGGGTAATCATCAGTTGAAT
GTCTGCATGGCCCCCACTTTTATTATAAATCTACAATGTATCATAGGAAGCCTTACTGTTCATCCACCCCTCACGTTATAGACAGGGAAACCAA
GGCCCTGGGAGGAGATGGGTCCTGGCCAGAATCACACTGAAAGTGGGTGGGAAAGTCAGACCTAGAACCCAGGTCTCCTTCCTCCCAGCTCCAG
ACTCCATCTCCCTCCTTTCTGAATTTTCATTCCATTGATAGATTTCAGGCCCTTTCATTAACATTCTGGAGTCATTTTCTGTCCCCCACCCTAA
CTGGAGTTTTTAGTATAAAGAGACAGGGAAAAGCTGTAAGTTAGAAATGAAGTCAGGGTGTGAAATTGGTGGAAAGGTGTGAGCTTAGGAGACC
AGCTGGGGCAGAGTGACCGCCAAGCCTTGCTCTCCCCATCTGTGCCATTGTAAGAAAAGATGGAGTCACTCCCTGCACCCTCTTCGCTCTTAGG
TACCATAAGATCTGTCCCCCAACTCTCCCAGATCTGTGACCCCACATTCTCTCTCCATAGGTGGAAAAGAGGCTGCTTCAGGCACCACACCTCA
GAAGTCCCGGAAGCCCAAGAAAGGGGCTGGGAACCCCCAAGCCTCAACCCTGGCGCTGCAAAGCAACATCACCCAGTGCCTCCTGGGCCAACCC
TGGCCCCTGAATGAGGCCCAGGTGCAGGCCTCAGTGGTGAAGGTCCTGACTGAGCTGCTGGAACAGGAAAGAAAGAAGGTGGTGGACACCACCA
AGGAGAGCAGCAGGAAGGGCTGGGAGAGCCGCAAGCGGAAGCTATCGGGAGACCAGCCAGCTGCCAGGACCCCCAGGAGCAAGAAGAAGAAGAA
GCTGGGGGCCGGGGAAGGTGGGGAGGCCTCTGTTTCCCCAGAAAAGACCTCCACGACTTCCAAGGGGAAAGCAAAGAGAGACAAAGCAAGTGGT
GATGTCAAGGAGAAGAAAGGGAAGGGGTCTCTTGGCTCCCAAGGGGCCAAGGACGAGCCAGAAGAGGAGCTTCAGAAGGGGATGGGGACGGTTG
AAGGTGGAGATCAAAGCAACCCAAAGAGCAAGAAGGAGAAGAAGAAATCCGACAAGAGTGAGTGACCGCTTCTCCCAGCCCACCCCAAGGGCTG
CTGGGCACCCCACGGGGGCGGGAGGGACCCTCAGCCAGCACCTGGTCTCATTCCTCCCATGTAGAAAAGAGAGGCTGAGACCAGCCTGGCCAAC
ATGGCAAAACCCCGTCTCTACTAAAAATACAAAAAATTAGCTGGATGTGGTGGCGGGCACCTGTAATCTCAGCTACATGGGAGGCTGAGGCAGG
AGAATTGTTTGAACCCGGGAGGCAGAGCTTGCAGTGAGCCGAGATCACGCATTGCACTCCAGCCTGGGTGACAGAGCAAGACTGTCAAAAAAAA
AAAAAAAAAAAAAAAAGGCTGAGAAGTGGTTTGTCCAGAGTCACAGGGGCAGCTTGTGGCAAAGCCCGAACTCGATTCCAGAGCACTTGAGTCA
GCACTGCCAGGAATGCTCCAGAAGGACGAGGAGTCATGTCCTCTTTCCCCAAGCCCAGTGCAGCAGATCTTGGAGGAACCTGCTTCTCCAGCCA
GCTCCTGGGTGCAGAATGGACCAGGAACCAGACTGTCAGAATCCTCATCCCAAACCCCATCTTGTCTCTAGACCTCAGTTTTCCCTCCATAGTA
ACACTGGCCCTTTACTGAGTGCTGTTCTAGACTCCGCATGCAGCAGGACCTCATTTAGGTAGAAATGAGACTCAGCAGGTAGAAAACTGCGGCT
CGGGCTCGGAGAAGTAGAGATGCTGTTCAGGGCCACAGAGTTTTGCTAGGTGGAGCCAGAGTCAAACCTAGGCAGCACTCTCGAGCATCGCCCT
GGACGGTGGGTTGCCAGTGACGCCCGAAGAGCCAGCTCGTTCTGGAACAAAAGTCGGGCACCCTCAGAGCAGGACTTGCAGCCCCTTGGTGCTC
AGAACTGTGCTAGATTTCCATTTACAGTAGGGATAGATAGTGTCCATTTAAAATAAATTTGCGGGAGTTTTAAAAAGTGAGTTGAGCTAAAGAA
AAATGGTAGTCATCCAGGTGGTCCATGGATGGGACAGAAATCATGAGCTGGCCTCCAGGGGGCAGGTGAACTGGCCCTGCTGGGTGATCACCAC
AGCTGCCAGCCTGTTTTATTTTTATTTTTTGAGACAGGGTCTTGCTCTGTCACCCAGGCTGGAGTTCAGTGTCAGGAACGCAGCTCACTGCAGG
CTCAGCCTCCCAAGCTCAAGCGATCCTCTTGCTTCAGCCCCCCAAATAGCTGGGACCACAGGCGTGCACCACCATCCCCAGCTAATTTTTGTAT
TTTTTGTAGAGACAGGTCTCACTATGTTGCCCAAGCTGGTCTTGAACTCCTGGGCTTAAGCAATCCACCCGCCTCAGCCTCCCAAAGTGCTGGG
ATTGCAGGAATGAACCTCCACGCCCCGCCCTGCTGGCCTGTTGTGATGGCTTCTGGTGGTGTGGGGAAAGCTGGGAGCCCTGCCCTGTGCACCT
CCCAACATTGACCCCAGCACTTAGGATTACCATCTGTTGTTCAGGAACTTTACTTTACTTCCCTTAGGAAAAAAAGACAAAGAAAAAAAAGAAA
AGAAGAAGAAAGCAAAAAAGGCCTCAACCAAAGATTCTGAGTCACCGTCCCAGAAGAAAAAGAAGAAAAAGGTAGAGAGTTCCTGGGGTGTCTC
AGGCCAGAAAACAGACCCAAACCCAAGCCCCCTCCTACACACCCAGACCTGGTTCCTGCCCCCTTCCCATTTTCGGGGCCCAGTCGGGGGCGTC
AGGGGTTGTGACACACCAGGGAAGAGGCTGGCAGGGCATGTTCCCTGAGCACATGGAAGCCGTAGAAGTGGCCTGCCGGAGGTAGAAGGGTGTT
GGGGAAGGAAGGAGAGACTCCCCCCAGCACAGGGAAACCAGGTAGACTGTGGCAGGTTTCCAGGTGAAAGGAAACGTTGCCTGGGCTGCGTGTG
CCCCAGCTGGCAGGCTAGCCCTGAACAACAGCTGTCCTCACGGCCCGGGGCCTTAGCACCTCAGACACAGATGAAGTTCTCACCTTGGCCAGCA
GGGCCTCAGCTGTATTTAGGAGAGCTGAACATCTGTTTGCCTCTGCCCTTGGAGGTCGCTGCAGACCCAGTATCTATTCTAGGGGAATTCACTA
GTCCTCAGGAGGTGGGGGCAGCAGTGGGTGGGGAAAAGCTGCAGGTCTGAGAGCCTCTCGTACTTCCCTCCTCACAGAAGAAGACAGCAGAGCA
GACTGTATGACGAGCACCAGCACCAGGCACAGGTACGCTTCCCAATCATTCCTGAGCATTCAGGGTGGGAGGACAGCTCTGGTGTCCCCTGTGG
TCCCAGAGAGCCAGGCAGACCTGATAGGTGGGCTCTAAGGGGAAAGGAGGCTGGGGAAAGAGGTTCTGTTGCCAAGGGTCCATGGGGCCACTGG
GGTGACATCAGGAGCTGGCACTGCAGCATCAGAAGGATCCAGAACAGTAATGGCAGTGGTGACACCGAGTGCACCTGGGTGCAGGGGTTGTGCA
TGCTTTAGGGGGTGTGCCCTGGGATGGAGCCTGTTTGTAGGTGAGGTCGGATACAGCTTGGTTCCTCCTTCACTTGTGAGCGGTTCATTTTCCT
CATGATCCTGTAACTTGGGGGCATTTCCTTTTGGGAAGGAAGATGGTTTACTCAAGATGAGCTATCACCATGGATCTATGCTTCCCTATGGAGG
ACAGGCCCTGCCAAGTGGTGGTGACAGTTAGCTGCTCCACCTGGCTCTGCTGGCCCTGTGGTCACCCAGCCTCCTGTAAATGGGGGTGATGCAG
AGCCCGCCTCCTGGGCCAGTGGTGAGGCTTATGTGAAGCTAAACTAAACACTAAAGGACTCAGCCCAGTTCTCAGAACATTCCAAGTGCTAAAA
AATGTCCACTGTGGTTAATGCCTCCCCCCTGCATTGGAGATGCCAGATTTCATTTTCCTTGCAGGGGAAAAGGGTCAGGAAATTTGACATCTGC
ATGTGTTTCCTTCCCCTAGGGATTTCCTAGCCGAGCAGTGGCCATCCCCATGCCTCTGACCTCCACCGACCTCTGCCCACCATGGGTTGGAACT
AAACTGTTACCTTCCCTCGCTCCACAGAAGAAGACAGCCAGCTTCAGGGGTCCCTGTGCTGGCCAAGCCAGTGAGCCTGCGGGGAGGCTGGTCC
AAGGAGAAAGTGGACCAGCTCCCATGACCTCACCCCACTCCCCCAACACAGGACGCTTCATATAGATGTGTACAGTATATGTATTTTTTTAAGT
GACCTCCTCTCCTTCCACAGACCCCACATGCCCAAAGGCCTCGGGACTTCCCACCACCTTGCTCCACAGATCCAGCTAGGCCTGACCTGTGCCT
CATCCCGTGCCGCTCGGTCTCTGGCTGATCCCGAGGCTTTGTCTTCCTCTCGTCAGTTCTTTTGGTTGTGTTTTTTGTTTTTTTTTTAATAACT
CAAAAAAAAAATAAAAGACTTGGAGGAAGGGTGCAAGCTCCCAGTGCATCTGGGGCACATGTTTCTTGGAAGGGACTGTCTCGCCGACACTCGG
GATTCCCTCTTGCCTGCATGTTGAGGCTATGGGTGACCGTGGTTAGTGGGACAGGCAGTGAGCAGTGAAGTGCCTGAGTGCCGAATTGGTGGGG
AAGGCTTCCTGGATGAGGTGGCATCTAAACTAAAGGATGGAACTAAAGGATGGTCATGGTTGATTTCCTGTTGCCAGAGTAAGTCATGTAAATA
CCCAAAGACAAGAAATGGAAGGGGCTGAGAATCCTCGAGTTAGGCCTCGAGCTAGGGGCAGGCTCCCTGGAGGTGCCTGGTGAACAGCTGTGGC
AGGCACAGCAGGTCTAGGAAAATCGGAAGTGAGTTGCCCCTCAAATCGAGCTCACCATGGTTCTGTGGTGGTGAGGGCAAGAAGTGGATGAACA
TGAAAGACAGGATGATGTCTTGGGTGTGGACTGAGGGAGGGGAGGATAGCAAAGGAGGTGGTCTGCTGCTCCAGCCTGGAAATGCAGAAGCCC
ACTTCCCTGGGCCCAGAGCCATGTGGAACACCAGGGGTCCTGGAGATGGTCTCCAGAGCTTTTAGCCTCATGCTGGAAACAGCCAGAGGCAGTG
CAGAAAAGGAATGCATGGCTGGAGGTGTCGGAGACCCAGGTTGGCATCCAGCTGCACCCTTGACTCGGGGCCCGGGGCCAGACCCTCCACCATC
TGGCCTCTCTCCTCTCAAGCATGTTGCTCTAGAACAGAACTACAGAGTAGTGTCCTCATTGGGCCTTGTTGACCCACAGCAAGACTTTAAACTT
AAAAATGGGGTGATTTCACTGCAGTTCTTGATTTCTGGCTCCTCTTAAAAAGATAACATCTGGCAACGGGGGCTGTGCTTCCTCCTGGCAGCAA
CCAGATAGAGCTGAGTAGCAGTCAGCCCCTTAGGTAAGGCATGGGCACCCAGTTCTCCACAGTCCCCTACTTCCTGTCCATCCTGACACGGAGG
CCCTATGTCGGTTGCCATCTGTCATCACATTCACACTGTTCTTAGTATAGAAATGAAATTGGGGACATAGGAAAATGAAAGCTGTACCAAGAAG
AGCTTGTGCTTAAACTAGGCAAGAGCCTATTTCTTTTTGGAAGTAGAGTGTTCGTGTGTGTTAAACATGTAATCAAGTATGTCTGTGTCCAAAG
ACTACAAGCCCCCCCCCAGGCCTGCCTGGCCCCGCAGGCATCTCTGTGGGTTACTTGGGCTGCGTTCTCCCACCTCAGTGACTGGATTTAGCGT
CTTTAGTTACCTCTTTAGGATAAATGAACTGGATCTCAACAGAAGAGCCAGGAGCCAGAGTGTTCTAATTACTACCTTTTATTCTAATGTGAAC
CATGGCCCTGAAAGCTGATAACAAGCTTGGCTGAGCAGAGGGAACTAGGGGTCGGCAGAAAGGATTATGGGTGGAAAACATTGGCTCTTCCTTG
GGGAGTGATGCTGGGGAAAGGGAAGAGAGTGGCTCAGCCTGCAGGTAAATAGGCTAGAAAAGCCAAGGCCAAAGGCTGGAGGGGAGAGGACAGT
CAGCATGTCCAGCCTGGGGTCTGGGTGTAGGGTTATCCCTTCTCCCTGTGCCTTCCCATCTCGTCCATGAGCCTAGGTCTTGGAGCCTTGTGTT
GGAGGCTGCTGTGATGTCAGGAACGGGGATCTGTCTAGCTTTTGGCCACTTCCTGGGACCTCACGCCCCTGTTGACAGATGGAGATTGGGCAGC
AGGGCCTTGCTGCATTGTTATCTGCTGTTCCGACTTGGTTTGTCTTGTCCAAGGGTGACGAAAGAGCCAGGCACCAGGGTCTCATGGGATGAGG
TACAGGGTGGGAGATGGGGGAGGGGCTGGGGGCTGAAGGGAGCAAGAAAGCTGTAGCTGTGTGGGGCTGGCAGGATGTTGAAGACCGCCTCTGC
TGCTCTCACATGGGGACTGGAGAGAGAAGCAGCAGGTTGAGGTTGGGGTCACCCATTTGTTGTCCCTGCCCCTTTCTAACATCCTGGACCTGCA
GAGCAGTTAAGGACTGTCCACCCTCCCCTGCCCCCTGCTCAGGTCCAATAATGACCATCATGGATTTGTGTAACTCCCCACAGCCCCCCAACCC
TGTTCCTTCGTCTGTGAAATGGACATCCCAGGAATGTTGGAGAGTGGCCCAGCGTCACACTCCTTCACCTGCCCACAAAGGAGCTGGCCCTGCT
GGGGATGAGCTGCTGGTGACCAGATGCCCCTCTGCAAGGCCCTTACCTGGGCCCAGATCCTGCTTGGTCACACCCAGCCCAGAAGACGGGTCCT
CCAGTTCCAGTGACTCTGCAAAGGAGCAGCAAGTCCGTTTGTCACTTGAAGTGCAGCCTACCTGACCCAAGATGCCTGAGGGCAGTGCTTCCCA
CCTAGCCACAGGCTTAGTGCCTGGGAAAGCAGGTACCCAGGGAAGGACAGCTGCAAGCAGCAACAAAGAGGTGTAATGGGGGCTCGAGTGAGAA
GGGGGTGATGAAATGAGATGGGTGGGTCTGTAGGACTCCTGGATGCTAGAGGATGGCGCGTGGATGGATGAAATTCACAGATGAAAGGACTGAC
GGATAAGTGAACAAATATGAGTGCATGTAAGAGGAGAGATGATAAATGGACAAATAGGAATGCACAGGTGGGTGGATGGATAAAGGGCTGGACG
CATGACTACGTCACTGCCCCCAGGAGCTGCCATCCTGGGTGTCCTGGTCCCATGTGCTTCAGAGGGGACCTCTTGCCCCTCAACCTTCCTAGTC
CTTCCTGGTCCTCTGACACTGGCACAGTGCCACTGCTTACCACTGCAGTTATGGTGCCCGCGGCTTCTGGTGTTGGGGACCTCCGTGCCGTTGG
GGAAGACACACCAGCAGTAGCCGATGCTCCCATAGCACTGGAGTGGCAGATAGTTGCCGTTCTCGTCGCACTTGGGCCTGAATGAACCCGGGTG
GACAGCAGGGATGTGGCTGACCTCTTCCTGGCACTTGGTCAGTACTGAAGCGACAGGCATGATGAGGACACAGTGAGTGAGTGAGCTCTGAACC
AGGGTCTGAGCAGAGCTAAAGACCCACACACCACTGCTGTGGGCTTAATGCCTTCTTCCCAAGTGGCTTTACTCACTCCAGCCATCACACGGAT
GGGAAAACTGAGGCCTAGACCAACAAGGTCTGAGATCAAAAAGGAAGTGAATGGCCATGTGTCCAATTCACCTCAGAAGTTGATGGTTTCTTCT
GACACTCAAAGCCCATTACAGTTTTGGGCCAGGCACAGTGGCTCATGCCTGTAATCTTAGCACTTTGGGAGGCCGAGGTGGGCGGATCACTTGA
GGTCAGGAGTTCAAGACCAGCCTGGCCAACATGGTGAAACCCTGTCTCTACTAAAAATACAAAAATTAGCTGGGCGTGGTGGCACATGCCTATA
ATCCCAGCTACTCGGGAGGCCGAGGCAGGAGAATTGCTTGAACCCGGGAGGCTGAGGTTGCAGTGAGCTGAGATCGCACCACTGCACTCCAGCC
TGGGCGACAGAGCCAGACTCTATCTCAAAAACAAACAATCAAACAAAAAAACACCATTACAGTTTGGAATTGCACTTGCGATTCTGATTCTAGA
GAAAAGCAATGGGCACCTTGGTAAGTCTAAGCTTCCTGTTACTGGGAGTTCTCAAGTAGAAATGGGGTTTACCGTTTGTTGGAGATGTCTTAGA
GGGGTTTAAGCATCAGCTAGGAGTTGGAGATAGCATGTAAGGTGCCTTCTATGAATCTGATGCAGAAACTGAGGCCCCAGGCAGGGTGGCCCTT
TGACCTTCTACTTCAGGACCCTGAATCCTAGAGGGAAGGGGACTTGTCCAAAGTTACATAGCAAATTAGAGACAGCACCGGGATAGAAACAGCA
CTCTGTCTTTGGGTCTAAAAACATCCTAGTCTGCATTGTCCATGAGGTGGGAAACGCCCAAGGCTCTCTTGGCAGCAGCTGTGACCTTGGCGCC
CTCACTGGATTGCGGGCAGCACTGGGGCAAGGTCATTACAAGGATTCCTTTCTGCAGTGTTTCGGGCAGGTTCTACCCCAGGCACCCTGTGCTG
TACCATCCTCTATGAGAGAGGTCACCTGTACCCTGTGGTGACAGGCCTCTGTGGGCCCTTAGGAACCCTTAGACAAGATGGGGGTGGGTCAGGG
GAGGGGGACAGCCAAACCAAGAAGGAACAGACAGGAGAGAGAGAGAAGTGAGAAAGAAGTGAGAGCTGAGAGGATGGGAGATTCCGCGGTGCTG
GGACCAGGGAGTGCTGGAGGCCCCGGCCTCAGCAGCTTCAGGAGAGCCCCGAGGGTCCAGAAGCCCTGGCACGCTAAAGCTCCCACTCCCTGTA
CCTTTCGGTGGAGCGTCAGTGGGCTTTTGCTCCAAGGAGTGCCTGCTCATTTCAAACAGGAGCCAATGGTGCATCCAGCTCTCAAAGACCTAAT
ACGGATAGAGGTGGAGGTCAGGTTCGATGGCCACCACCAAGCCCCTACACTGCCTGGCTTTGCCCCTGGGGACAGAAACATGCAGGGCCAAATA
GGGCCCCAACACCCGGGTCCAGCTGCAGCCTGGTTCCCTCTTAAAGACCCCTGGGGCTGAACCCCACATACTCTGCAGCATCCAGGTTTTGGAA
GTGCTGGTAAACCCCTTCAGCTTCCTGAGAATGGCTTCCCCAGCCCACCCTCACCCCACCTCTCCTAGCCCTGCCCTAGACACCAGAGGAAAGA
GAGAGTTCCCATGCAGGGAAACCTGCTGACCTTCCAGTCTATGGTCTCCATGGTGTTCTTAAGGTGTCTCAGGTTCTCCGGGAAGCTCCCCTTC
AGTGGCGGGTACACCTTCAGGGGGTCAGCATTCTACAGGGTAGCAGGGCAGGAAGGATGGTTCAAGAAGAGCTCCCTGGCAGGCAGTGTGAAAT
GTGTAGCCCACGTTCCCTTGGTCTTCCATTCTGAGGAATGGACCAGAGCTACACCAAGCCTAAACCCTGGACTTCAGCCATGATGGGAGGGGAG
GGGAGGGTACGGGTAAGGGTAGTCCAGCTGTTCAGCTACGAGACTGCAGCGTTGCGAGTCGAGTTCCAGATTCCAGGCCAGCTCTACCAACAAG
CCCCAGTGTCCTCCCTGTGGATGGCTGCTCAGCTTTCAGGTCTTACCTGCTCCAGCAGGTGAGGACCCAACCTTGACTGCCCCCAGCTAGGTTG
AGTCCTCTGCCTCTTTCTTCACAAAGCCCCCTGCACTTCTGTCATCACCTGGTCACTTGTCTGTCTCCCACACTAGCCTGGGATCCCCATGGCT
TCAGGAGCTGGGTCCAATTGCTCCCCATTGTGGGCCCAGAGCCTTGCACAGTGCTGGCATAGGACAGACACGCAGTACAAATTTGGGGAGAAAA
AAAGGGTGTCTTGAGCAAGTCACTTCCAAACTTTGAGCCTCAGTTTCTTTTTTTATTTTTATTTTATTTTATTTTTTGAAATGGAGTCTCACTC
TGTCACCCAGGCTGGAGTGCAGTGGCGTGATGATCTCGGCTCACTGCAACCTCCACTTCCCGGGTTAAAGCGATTCTCCTGCCTCAACCTCATG
AGTAGCTGGGATTACAGGTGACTGCCACCACATCCGGCTAATTTTTGTATTTTTAGTAGAGACTTTCACTATGTTGGCCAGGCTGGTCTCGAAC
TCCTGGCCTCAGGTGATCTGCCTGCCTAGGCCTCCCAAAGTGCTGAGATTACAGGCATGAGCCACTGCACCCAGCCGAGCCTGTTTATCTCAAA
AATGCAGAGGCCAGACTACATATAACATTTAAGGGTCACCCTTGTATTCAGTAATCCAGAGAGCACATGTTGAAAGTCTGCCATGAGCCAGGTA
TACAGGCCTTGGAGCTTGGCCCGGCCAGGGTCCTGGGTGGGCAGGCAGGACCACCCAGCTAGCTCCCTGCACTCACCTGGAGCAGGTGCATCAC
ATGGTCCTCTGTCATGTTGCCATACTTGGTGGCATTCTGCATGGGCTGTGGGAGGAAGTAACAGAAGGTTACCAGAGCTGGTCCCTGGAGCAGG
GGGTATGGAGCAGGAGCCGGTTGCTGGGATCTAGGTAAGAGACTGGAATCGCAGGCACTTGGTCAATGGTTCCATCTCTGCCACCTCCAAACAT
GAGAGCTCGAGCAAGTCACTTTAGCTTCCTGCCTCAATGTCCTCATTGGTCAAACTGGCATAAGCTTTCCTGCCCAGGGTTTTGTCCACAGCAC
AGTATTGTGAATATAAATATTGTCAATGCATGATAGCAAGGTCTTTGAGAATGGGAGAGGACAGTGAGATGCTATAGGAAATGAGAAGCACTTT
GTAAACTGTAGAGGGCTGAGCCTTCCTCCCAGGGATGGCACCAAGAGACTCTTCACCTGTGCCCTCCACACCTGGGGAGCCCGATTCTTCCTGA
GGCAGGCACTGTCCCTCCCGCAGCAGTCAGGACCCCTCCCCAGGTGGGCTCTCTTCCCTGCCCCTCCCCTTGGCACTGTCCTCCATCCAGGCGG
GATAACCTTGCCCCTCCCACCACCCTGGGGCTGTCCTTACCCCCTGGGGCAGGGCTCCCATGGGCAGCGCCTGCATCAGCAGCGGGGTGGCCAT
GCGCATCTTGCTCACAGGCTTGGGAGCTGTGGGGACAGGAACGAGGGTAAGTGTGGCCCCGGTGCCCAGGGAGGAGGGTATCAGACCCAGATGA
GGAAGGGCTGGAATTCCAAACCGGAGGGAGAGGACAGTGGGCCCAGCTGGGTGCAGGGATGCGGGTCTCTGAGTTGAGGGATGGTGGGAGGTGG
GGGGTATCAGGATGTAGGGGTGCACGCACGCTTGGGAAGCTTCATGCGCAGGTTCTCCAGCTGCAGGTTCTGGGAGGTGACTGTCAGTTTGTCC
AGCCGGCCCTGCTGCTGGTACAGGAAGTAGGCGGTGGTGGCCTGGCCAGCGAGGAGCAGAGTCACCAGGATGGAAAAGCCTGTGTACAGGGCTC
CGCGGCTGCACTTGCTGTGGGAGGTGGGGAGGATAGGTCAGAGGAGGATCCACAGTGGTGGCTGCCCCAAGGGCTGGCTAGGAATGGGGAAATG
TATACCCCCATCTCCATTAGACCCCTAAGCCACCCCTAATAAACAATGCATTTGAAGCACAAACAGTAAGCTGGCTATGGAAACACAGCTTGAA
GACCTCACCCCACCCCTCCTTCCAACCATCGATGTGCCTGAAGTCTTCCCTGGCAGAGACCCTGCAGCTGCCACGGGCCTGAGTTGAACAGAGA
AGAGCAAAGAGGCATGCTCTTTGTCCTCTCTGGACCTTAGGGTCCTGCCTGTGCGATGAGGGGTTGAGGTGGGGGGGTCTTCCCAGGCTCCCTT
TGCCGTGCGGATCTCCTGATGATCAGGGCTGCCCTCAAGGGGAACAGGTTTGGTTTTTGTTTTGTTTTGTTTTGTTTTGTTTTTTGAGACGGAG
TCTTGCTCTGTCGCCCAGGCTGGAGTGCAGTGGCGCGATCTCGGCTCACTGCAACCTCCACCTCCTGGGTTTATGCCATTCTCCTTCCTCAGCC
TCTCCGAGTAGCTGGGACTACAGGCACCCGCCACCACGCCTGGCTAATTATTTTGTATTTTTGATAGAGACGGGGTTTCACCGTGGTCTCGATC
TCCTGATCTCGTGATCCACCCACCTCAGCCTCCCAAAGTGCTGGGATTACAAGCGTGAGTCACCGCGCCCAGCCTTTTGTTTTGTTTTTTTTTT
CTGCTGGCAGGATCAACCAGGTATGTTATTTTTTTTAATTGACAAAACCAAAAAAAATTGTATGTGCTTACCAAGTAAGGGGAAGAGGGTTTTC
ACCCCTCTGCCCAACCTCCCACCCTCACAGTGAAATCAGATTTAAGGAGAAAAGGTAAGGAAGTGGGACCGGGACAGCAGAAAATGTAGCCCAC
CTGGGAAGAAGTGGGGCCTGGCCATTTTAGGAGTGGGCTGGGGGAGGCTTGGACAATGTCAGGCTGAAGCAGGGCCAGGCACCACCCACTCTGG
GGCCATCCTCTGCCCAGGCTCTCCATGTCTTTGAGCATGTCCCTGCCAATCTCTGTGCCTCTGTTCCCTCATTTGTCACATAGGTGCCCCCACC
TCTGCGATTCTAGAATTTGGTGGGTTTCTTCTTTTAAGCAAGATCTCACTGCAAGCGCCAAAGTATTTCAAATGGTAATATCCATCCTGCAGGG
GGTGGGCAGGAGTATAGGTGCCTGAGCCTGTGTGGCCTGGATGAATTTCTCATGCTCCCTGGACTCACCATCCAGACCCTCGGACCTCAGCAAG
CCCTTGTATCTGTGGGACTTGGATGCGACTGGGGGGTGCATGCATATGTTACAGTACAGGGGATGTAAGGATCCTGCCCCTACACCCCAGGAGT
TTCTGATCTGTTCTTTGACGACCCCAGGGGTGGGACTACTAAGACCCTAGTCCAGGTGTTGCCAATGTGAAGGTGTTGGGAGGTGATCGGGCCA
CAAGGCCTTCTCCCTCACGAATGGGATTAGGTGCCCTGTAAGACGGCTTGATGGAAGGAGTTGGTTCTCTCTTGCCCTTCTGCCTTCCACCATA
TGAGGACACATCAAGGAAGCCCCTCACCAGGCTGGGTGTGGTGACTCACGCCTGTAATCCCAGCACTTTGGGAGGCCAAGGCAGGTGGATCACT
TGAGGCCAGGAGTTCGAGACCAGTCTGGCCAACATGGCGAAACCCCGTCTCTACAAAAAAATACAAAAAACTTAGCCAGGCATGGTGGCGTGCA
CCTCTAGTCCCAGCTACTTGGGAGGCTGAGGTGGGAGAATCACTTGAACCCGGGAGGCGGAGGTTGCAGTGAGCCGAGATCACGTCACTGCACT
CCAGCCTGGGTGACAGAAGGAGACTGTCTCAGAAAAAAAAAAGAAAGCAAGCTAGGCCGGGCGCGGTGGCTCACACCTGTAATCCCAGCACTTT
AGGAAGCCAAGACAGGTAGATCACCTGAGGTCAGGAGTTCGAGACCAGCCTGGCCAACGTGGTGAAACCCTGTCTCTACTAAAAATGCAAAAAT
TAGCCGGACGTGGTGGCGAGCACCTGTAATCCCAGCTACTCGGGAGGCTGAGGCAGGAGAATCCCTTGAACCTGGAAGGTGGAGGTTGCAGTGA
GCTGAGATCATGCCATTGCACTCCAGCCTGGGCAACAGAGTGAGGCTCCATCTCAAAAAAAAAAGAAAAAATTAGCCGGGCGTAGTGGTGGGCA
CCTGTAGTCCCAGCTACTCAGGAGGCTGAGGCAGGAGAATCACTTGAACCTGGGAGGCAGAGGTTGCTGTGAGCCAAGATCACACCACTGCATT
CCATCCTGGGGGATAGAGTGAGACTCTGTCTCAAAAAACATAACAAAAAAAAAAAAAAAAAACAAGAAAGCCCCTCCAGAACTGTGAGAAATAA
ATTTCTGGGTGTTTTTTGTTTGTTTGTTTGTTTGTTTGTATATTACGCAGTCTGTGGTATTCTGTTAAAGCGGCACAAAACGGACTAAAACAGA
CCCATTGGGAACTGCAAGGATGAGGCTGCACCCTGCCTACTCCAGCAACCAGCAAGGATGGTTTAGGACGCTCCAGGAGTCCCTTGTGCCTGGT
AACTCATGACATCACAGACTGGCTCATTCTCATTGGCTTGCTGCGTGCCAGTCTGTGCTTTCTGGGATAAGGCCCAGCCCAAGGGAGAGTTATT
CGGGAAGGAAACAGCAAAAACCCGACGGTGGGCGGGGGGCGGGGGGGAAGTGGGGAGACCAGACAGGAAGGGAAAACAAATTCTACTTCTTGGC
CTTCTCAAGGAACACGGGTGGCCTAGCCTGTGTTCAGGCTCTCCAGCCTCATCCTCCACCTGTTCAGTGCTTCTGCAACCCCACACCCAATTCT
AGCCCCAGCTCTACCTCCCACACACGACATGGGACAGTACCTTCCCCTCTCTGCACCACAGTTCCTCAGGAG
HUMAN SEQUENCE-mRNA
TAAGGGCGCGAGGGAAGTGGCGGGCGGGGACTAAGGCGGGGCGTGCAGGTAGCCGGCCGGCCGGGGGTCGCGGGTATGGCCGAGGCCAGGAAGC
GGCGGGAGCTACTTCCCCTGATCTACCACCATCTGCTGCGGGCTGGCTATGTGCGTGCGGCGCGGGAAGTGAAGGAGCAGAGCGGCCAGAAGTG
TTTCCTGGCTCAGCCCGTAACCCTTCTGGACATCTATACACACTGGCAACAAACCTCAGAGCTTGGTCGGAAGCGGAAGGCAGAGGAAGATGCG
GCACTGCAAGCTAAGAAAACCCGTGTGTCAGACCCCATCAGCACCTCGGAGAGCTCGGAAGAGGAGGAAGAAGCAGAAGCCGAAACCGCCAAAG
CCACCCCAAGACTAGCATCTACCAACTCCTCAGTCCTGGGGGCGGACTTGCCATCAAGCATGAAAGAAAAAGCCAAGGCAGAGACAGAGAAAGC
TGGCAAGACTGGGAATTCCATGCCACACCCTGCCACTGGGAAGACGGTGGCCAACCTTCTTTCTGGGAAGTCTCCCAGGAAGTCAGCAGAGCCC
TCAGCAAATACTACGTTGGTCTCAGAAACTGAGGAGGAGGGCAGCGTCCCGGCCTTTGGAGCTGCTGCCAAGCCTGGGATGGTGTCAGCGGGCC
AGGCCGACAGCTCCAGCGAGGACACCTCCAGCTCCAGTGATGAGACAGACGTGGAGGTAAAGGCCTCTGAAAAAATTCTCCAGGTCAGAGCTGC
CTCAGCCCCTGCCAAGGGGACCCCTGGGAAAGGGGCTACCCCAGCACCCCCTGGGAAGGCAGGGGCTGTAGCCTCCCAGACCAAGGCAGGGAAG
CCAGAGGAGGACTCAGAGAGCAGCAGCGAGGAGTCATCTGACAGTGAGGAGGAGACGCCAGCTGCCAAGGCCCTGCTTCAGGCGAAGGCCTCAG
GAAAAACCTCTCAGGTCGGAGCTGCCTCAGCCCCTGCCAAGGAGTCCCCCAGGAAAGGAGCTGCCCCAGCGCCCCCTGGGAAGACAGGGCCTGC
AGTTGCCAAGGCCCAGGCGGGGAAGCGGGAGGAGGACTCGCAGAGCAGCAGCGAGGAATCGGACAGTGAGGAGGAGGCGCCTGCTCAGGCGAAG
CCTTCAGGGAAGGCCCCCCAGGTCAGAGCCGCCTCGGCCCCTGCCAAGGAGTCCCCCAGGAAAGGGGCTGCCCCAGCACCTCCTAGGAAAACAG
GGCCTGCAGCCGCCCAGGTCCAGGTGGGGAAGCAGGAGGAGGACTCAAGAAGCAGCAGCGAGGAGTCAGACAGTGACAGAGAAGCACTGGCAGC
CATGAATGCAGCTCAGGTGAAGCCCTTGGGGAAAAGCCCCCAGGTGAAACCTGCCTCTACCATGGGCATGGGGCCCTTGGGGAAAGGCGCCGGC
CCAGTGCCACCTGGGAAGGTGGGGCCTGCAACCCCCTCAGCCCAGGTGGGGAAGTGGGAGGAGGACTCAGAGAGCAGTAGTGAGGAGTCATCAG
ACAGCAGTGATGGAGAGGTGCCCACAGCTGTGGCCCCGGCTCAGGAAAAGTCCTTGGGGAACATCCTCCAGGCCAAACCCACCTCCAGTCCTGC
CAAGGGGCCCCCTCAGAAGGCAGGGCCTGTAGCCGTCCAGGTCAAGGCTGAAAAGCCCATGGACAACTCGGAGAGCAGCGAGGAGTCGTCGGAC
AGTGCGGACAGTGAGGAGGCACCAGCAGCCATGACTGCAGCTCAGGCAAAACCAGCTCTGAAAATTCCTCAGACCAAGGCCTGCCCAAAGAAAA
CCAATACCACTGCATCTGCCAAGGTCGCCCCTGTGCGAGTGGGCACCCAACCCCCCCGGAAAGCAGGAACTGCGACTTCTCCAGCAGGCTCATC
CCCAGCTGTGGCTGGGGGCACCCAGAGACCAGCAGAGGATTCTTCAAGCAGTGAGGAATCAGATAGTGAGGAAGAGAAGACAGGTCTTGCAGTA
ACCGTGGGACAGGCAAAGTCTGTGGGGAAAGGCCTCCAGGTGAAAGCAGCCTCAGTGCCTGTCAAGGGGTCCTTGGGGCAAGGGACTGCTCCAG
TACTCCCTGGGAAGACGGGGCCTACAGTCACCCAGGTGAAAGCTGAAAAGCAGGAAGACTCTGAGAGCAGTGAGGAGGAATCAGACAGTGAGGA
AGCAGCTGCATCTCCAGCACAGGTGAAAACCTCAGTAAAGAAAACCCAGGCCAAAGCCAACCCAGCTGCCGCCAGAGCACCTTCAGCAAAAGGG
ACAATTTCAGCCCCTGGAAAAGTTGTCACTGCAGCTGCTCAAGCCAAGCAGAGGTCTCCATCCAAGGTGAAGCCACCAGTGAGAAACCCCCAGA
ACAGTACCGTCTTGGCGAGGGGCCCAGCATCTGTGCCATCTGTGGGGAAGGCCGTGGCTACAGCAGCTCAGGCCCAGACAGGGCCAGAGGAGGA
CTCAGGGAGCAGTGAGGAGGAGTCAGACAGTGAGGAGGAGGCGGAGACGCTGGCTCAGGCGAAGCCTTCAGGGAAGACCCACCAGATCAGAGCT
GCCTTGGCTCCTGCCAAGGAGTCCCCCAGGAAAGGGGCTGCCCCAACACCTCCTGGGAAGACAGGGCCTTCGGCTGCCCAGGCAGGGAAGCAGG
ATGACTCAGGGAGCAGCAGCGAGGAATCAGACAGTGATGGGGAGGCACCGGCAGCTGTGACCTCTGCCCAGGTGATTAAACCCCCCCTGATTTT
TGTCGACCCTAATCGTAGTCCAGCTGGCCCAGCTGCTACACCCGCACAAGCCCAGGCTGCAAGCACCCCGAGGAAGGCCCGAGCCTCGGAGAGC
ACAGCCAGGAGCTCCTCCTCCGAGAGCGAGGATGAGGACGTGATCCCCGCTACACAATGCTTGACTCCTGGCATCAGAACCAATGTGGTGACCA
TGCCCACTGCCCACCCAAGAATAGCCCCCAAAGCCAGCATGGCTGGGGCCAGCAGCAGCAAGGAGTCCAGTCGGATATCAGATGGCAAGAAACA
GGAGGGACCAGCCACTCAGGTGTCAAAGAAGAACCCAGCTTCCCTCCCACTGACCCAGGCTGCCCTGAAGGTCCTCGCCCAGAAAGCCAGTGAG
GCTCAGCCTCCTGTTGCCAGGACCCAGCCTTCAAGTGGGGTTGACAGTGCTGTGGGAACACTCCCTGCAACAAGTCCCCAGAGCACCTCCGTCC
AGGCCAAAGGGACCAACAAGCTCAGAAAACCTAAGCTTCCTGAGGTCCAGCAGGCCACCAAAGCCCCTGAGAGCTCAGATGACAGTGAGGACAG
CAGCGACAGTTCTTCAGGGAGTGAGGAAGATGGTGAAGGGCCCCAGGGGGCCAAGTCAGCCCACACGCTGGGTCCCACCCCCTCCAGGACAGAG
ACCCTGGTGGAGGAGACCGCAGCAGAGTCCAGCGAGGATGATGTGGTGGCGCCATCCCAGTCTCTCCTCTCAGGTTATATGACCCCTGGACTAA
CCCCAGCCAATTCCCAGGCCTCAAAAGCCACTCCCAAGCTAGATTCCAGCCCCTCAGTTTCCTCTACTCTGGCCGCCAAAGATGACCCAGATGG
CAAGCAGGAGGCAAAGCCCCAACAGGCAGCAGGCATGTTGTCCCCTAAAACAGGTGGAAAAGAGGCTGCTTCAGGCACCACACCTCAGAAGTCC
CGGAAGCCCAAGAAAGGGGCTGGGAACCCCCAAGCCTCAACCCTGGCGCTGCAAAGCAACATCACCCAGTGCCTCCTGGGCCAACCCTGGCCCC
TGAATGAGGCCCAGGTGCAGGCCTCAGTGGTGAAGGTCCTGACTGAGCTGCTGGAACAGGAAAGAAAGAAGGTGGTGGACACCACCAAGGAGAG
CAGCAGGAAGGGCTGGGAGAGCCGCAAGCGGAAGCTATCGGGAGACCAGCCAGCTGCCAGGACCCCCAGGAGCAAGAAGAAGAAGAAGCTGGGG
GCCGGGGAAGGTGGGGAGGCCTCTGTTTCCCCAGAAAAGACCTCCACGACTTCCAAGGGGAAAGCAAAGAGAGACAAAGCAAGTGGTGATGTCA
AGGAGAAGAAAGGGAAGGGGTCTCTTGGCTCCCAAGGGGCCAAGGACGAGCCAGAAGAGGAGCTTCAGAAGGGGATGGGGACGGTTGAAGGTGG
AGATCAAAGCAACCCAAAGAGCAAGAAGGAGAAGAAGAAATCCGACAAGAGAAAAAAAGACAAAGAAAAAAAAGAAAAGAAGAAGAAAGCAAAA
AAGGCCTCAACCAAAGATTCTGAGTCACCGTCCCAGAAGAAAAAGAAGAAAAAGAAGAAGACAGCAGAGCAGACTGTATGACGAGCACCAGCAC
CAGGCACAGGGATTTCCTAGCCGAGCAGTGGCCATCCCCATGCCTCTGACCTCCACCGACCTCTGCCCACCATGGGTTGGAACTAAACTGTTAC
CTTCCCTCGCTCCACAGAAGAAGACAGCCAGCTTCAGGGGTCCCTGTGCTGGCCAAGCCAGTGAGCCTGCGGGGAGGCTGGTCCAAGGAGAAAG
TGGACCAGCTCCCATGACCTCACCCCACTCCCCCAACACAGGACGCTTCATATAGATGTGTACAGTATATGTATTTTTTTAAGTGACCTCCTCT
CCTTCCACAGACCCCACATGCCCAAAGGCCTCGGGACTTCCCACCACCTTGCTCCACAGATCCAGCTAGGCCTGACCTGTGCCTCATCCCGTGC
CGCTCGGTCTCTGGCTGATCCCGAGGCTTTGTCTTCCTCTCGTCAGTTCTTTTGGTTGTGTTTTTTGTTTTTTTTTTAATAACTCAAAAAAAAA
ATAAAAGACTTGGAGGAAGGGTAAAAAAAAAAAAAAAAAAAAAAAA
HUMAN SEQUENCE-CODING
ATGGCCGAGGCCAGGAAGCGGCGGGAGCTACTTCCCCTGATCTACCACCATCTGCTGCGGGCTGGCTATGTGCGTGCGGCGCGGGAAGTGAAGG
AGCAGAGCGGCCAGAAGTGTTTCCTGGCTCAGCCCGTAACCCTTCTGGACATCTATACACACTGGCAACAAACCTCAGAGCTTGGTCGGAAGCG
GAAGGCAGAGGAAGATGCGGCACTGCAAGCTAAGAAAACCCGTGTGTCAGACCCCATCAGCACCTCGGAGAGCTCGGAAGAGGAGGAAGAAGCA
GAAGCCGAAACCGCCAAAGCCACCCCAAGACTAGCATCTACCAACTCCTCAGTCCTGGGGGCGGACTTGCCATCAAGCATGAAAGAAAAAGCCA
AGGCAGAGACAGAGAAAGCTGGCAAGACTGGGAATTCCATGCCACACCCTGCCACTGGGAAGACGGTGGCCAACCTTCTTTCTGGGAAGTCTCC
CAGGAAGTCAGCAGAGCCCTCAGCAAATACTACGTTGGTCTCAGAAACTGAGGAGGAGGGCAGCGTCCCGGCCTTTGGAGCTGCTGCCAAGCCT
GGGATGGTGTCAGCGGGCCAGGCCGACAGCTCCAGCGAGGACACCTCCAGCTCCAGTGATGAGACAGACGTGGAGGTAAAGGCCTCTGAAAAAA
TTCTCCAGGTCAGAGCTGCCTCAGCCCCTGCCAAGGGGACCCCTGGGAAAGGGGCTACCCCAGCACCCCCTGGGAAGGCAGGGGCTGTAGCCTC
CCAGACCAAGGCAGGGAAGCCAGAGGAGGACTCAGAGAGCAGCAGCGAGGAGTCATCTGACAGTGAGGAGGAGACGCCAGCTGCCAAGGCCCTG
CTTCAGGCGAAGGCCTCAGGAAAAACCTCTCAGGTCGGAGCTGCCTCAGCCCCTGCCAAGGAGTCCCCCAGGAAAGGAGCTGCCCCAGCGCCCC
CTGGGAAGACAGGGCCTGCAGTTGCCAAGGCCCAGGCGGGGAAGCGGGAGGAGGACTCGCAGAGCAGCAGCGAGGAATCGGACAGTGAGGAGGA
GGCGCCTGCTCAGGCGAAGCCTTCAGGGAAGGCCCCCCAGGTCAGAGCCGCCTCGGCCCCTGCCAAGGAGTCCCCCAGGAAAGGGGCTGCCCCA
GCACCTCCTAGGAAAACAGGGCCTGCAGCCGCCCAGGTCCAGGTGGGGAAGCAGGAGGAGGACTCAAGAAGCAGCAGCGAGGAGTCAGACAGTG
ACAGAGAAGCACTGGCAGCCATGAATGCAGCTCAGGTGAAGCCCTTGGGGAAAAGCCCCCAGGTGAAACCTGCCTCTACCATGGGCATGGGGCC
CTTGGGGAAAGGCGCCGGCCCAGTGCCACCTGGGAAGGTGGGGCCTGCAACCCCCTCAGCCCAGGTGGGGAAGTGGGAGGAGGACTCAGAGAGC
AGTAGTGAGGAGTCATCAGACAGCAGTGATGGAGAGGTGCCCACAGCTGTGGCCCCGGCTCAGGAAAAGTCCTTGGGGAACATCCTCCAGGCCA
AACCCACCTCCAGTCCTGCCAAGGGGCCCCCTCAGAAGGCAGGGCCTGTAGCCGTCCAGGTCAAGGCTGAAAAGCCCATGGACAACTCGGAGAG
CAGCGAGGAGTCGTCGGACAGTGCGGACAGTGAGGAGGCACCAGCAGCCATGACTGCAGCTCAGGCAAAACCAGCTCTGAAAATTCCTCAGACC
AAGGCCTGCCCAAAGAAAACCAATACCACTGCATCTGCCAAGGTCGCCCCTGTGCGAGTGGGCACCCAACCCCCCCGGAAAGCAGGAACTGCGA
CTTCTCCAGCAGGCTCATCCCCAGCTGTGGCTGGGGGCACCCAGAGACCAGCAGAGGATTCTTCAAGCAGTGAGGAATCAGATAGTGAGGAAGA
GAAGACAGGTCTTGCAGTAACCGTGGGACAGGCAAAGTCTGTGGGGAAAGGCCTCCAGGTGAAAGCAGCCTCAGTGCCTGTCAAGGGGTCCTTG
GGGCAAGGGACTGCTCCAGTACTCCCTGGGAAGACGGGGCCTACAGTCACCCAGGTGAAAGCTGAAAAGCAGGAAGACTCTGAGAGCAGTGAGG
AGGAATCAGACAGTGAGGAAGCAGCTGCATCTCCAGCACAGGTGAAAACCTCAGTAAAGAAAACCCAGGCCAAAGCCAACCCAGCTGCCGCCAG
AGCACCTTCAGCAAAAGGGACAATTTCAGCCCCTGGAAAAGTTGTCACTGCAGCTGCTCAAGCCAAGCAGAGGTCTCCATCCAAGGTGAAGCCA
CCAGTGAGAAACCCCCAGAACAGTACCGTCTTGGCGAGGGGCCCAGCATCTGTGCCATCTGTGGGGAAGGCCGTGGCTACAGCAGCTCAGGCCC
AGACAGGGCCAGAGGAGGACTCAGGGAGCAGTGAGGAGGAGTCAGACAGTGAGGAGGAGGCGGAGACGCTGGCTCAGGCGAAGCCTTCAGGGAA
GACCCACCAGATCAGAGCTGCCTTGGCTCCTGCCAAGGAGTCCCCCAGGAAAGGGGCTGCCCCAACACCTCCTGGGAAGACAGGGCCTTCGGCT
GCCCAGGCAGGGAAGCAGGATGACTCAGGGAGCAGCAGCGAGGAATCAGACAGTGATGGGGAGGCACCGGCAGCTGTGACCTCTGCCCAGGTGA
TTAAACCCCCCCTGATTTTTGTCGACCCTAATCGTAGTCCAGCTGGCCCAGCTGCTACACCCGCACAAGCCCAGGCTGCAAGCACCCCGAGGAA
GGCCCGAGCCTCGGAGAGCACAGCCAGGAGCTCCTCCTCCGAGAGCGAGGATGAGGACGTGATCCCCGCTACACAATGCTTGACTCCTGGCATC
AGAACCAATGTGGTGACCATGCCCACTGCCCACCCAAGAATAGCCCCCAAAGCCAGCATGGCTGGGGCCAGCAGCAGCAAGGAGTCCAGTCGGA
TATCAGATGGCAAGAAACAGGAGGGACCAGCCACTCAGGTGTCAAAGAAGAACCCAGCTTCCCTCCCACTGACCCAGGCTGCCCTGAAGGTCCT
CGCCCAGAAAGCCAGTGAGGCTCAGCCTCCTGTTGCCAGGACCCAGCCTTCAAGTGGGGTTGACAGTGCTGTGGGAACACTCCCTGCAACAAGT
CCCCAGAGCACCTCCGTCCAGGCCAAAGGGACCAACAAGCTCAGAAAACCTAAGCTTCCTGAGGTCCAGCAGGCCACCAAAGCCCCTGAGAGCT
CAGATGACAGTGAGGACAGCAGCGACAGTTCTTCAGGGAGTGAGGAAGATGGTGAAGGGCCCCAGGGGGCCAAGTCAGCCCACACGCTGGGTCC
CACCCCCTCCAGGACAGAGACCCTGGTGGAGGAGACCGCAGCAGAGTCCAGCGAGGATGATGTGGTGGCGCCATCCCAGTCTCTCCTCTCAGGT
TATATGACCCCTGGACTAACCCCAGCCAATTCCCAGGCCTCAAAAGCCACTCCCAAGCTAGATTCCAGCCCCTCAGTTTCCTCTACTCTGGCCG
CCAAAGATGACCCAGATGGCAAGCAGGAGGCAAAGCCCCAACAGGCAGCAGGCATGTTGTCCCCTAAAACAGGTGGAAAAGAGGCTGCTTCAGG
CACCACACCTCAGAAGTCCCGGAAGCCCAAGAAAGGGGCTGGGAACCCCCAAGCCTCAACCCTGGCGCTGCAAAGCAACATCACCCAGTGCCTC
CTGGGCCAACCCTGGCCCCTGAATGAGGCCCAGGTGCAGGCCTCAGTGGTGAAGGTCCTGACTGAGCTGCTGGAACAGGAAAGAAAGAAGGTGG
TGGACACCACCAAGGAGAGCAGCAGGAAGGGCTGGGAGAGCCGCAAGCGGAAGCTATCGGGAGACCAGCCAGCTGCCAGGACCCCCAGGAGCAA
GAAGAAGAAGAAGCTGGGGGCCGGGGAAGGTGGGGAGGCCTCTGTTTCCCCAGAAAAGACCTCCACGACTTCCAAGGGGAAAGCAAAGAGAGAC
AAAGCAAGTGGTGATGTCAAGGAGAAGAAAGGGAAGGGGTCTCTTGGCTCCCAAGGGGCCAAGGACGAGCCAGAAGAGGAGCTTCAGAAGGGGA
TGGGGACGGTTGAAGGTGGAGATCAAAGCAACCCAAAGAGCAAGAAGGAGAAGAAGAAATCCGACAAGAGAAAAAAAGACAAAGAAAAAAAAGA
AAAGAAGAAGAAAGCAAAAAAGGCCTCAACCAAAGATTCTGAGTCACCGTCCCAGAAGAAAAAGAAGAAAAAGAAGAAGACAGCAGAGCAGACT
GTATGA

TABLE 9
MOUSE NOMENCLATURE
ICSGNM Pim1
Celera mCG21141
HUMAN NOMENCLATURE
HGNC PIM1
Celera hCG33220
MOUSE SEQUENCE—GENOMIC
TAGGGAACACCAATTGAGAAAACACCTCCATAAGATGCAGTTATAAGACATTTTCTTTTTCTTTTCTTTTCTTTCTCCTGGAAATCACTTTGTA
GACCAAGCTGGCCTCAAACTCAGAAATCTGCCTGCCTCTGCCTCCTGAGTGCTAGTATTAAAGGCGTGCACCACCACACTGGGCAAGACATTTT
CTTAATTAGTGATTGGTAGGGGAGGGTCCAGCCTAGTGGGTGGGGCTATTCCTGGGCTGGTGGTCCTGGGTTCTGTAATTAAGCAAACTGAACA
CACCATGAAGAGCAAGCCAGGAATGAGAACTCTCCATGGTCTCTGCATCAGCTCCTTCCTCCAGGTTCCTGCCCTGCTTGAGTTCCTGTCCTGA
CTTTTTGATAAACAGGTATGTGGAAATGTAAGCTGAATAAACCCTCTCTTCCCCAAGTTCTGGCCATGGTGGTTCATCCGGGCAATTGTAATGC
TAACTCCGAGTCTTTCTCTGGGTTAAATATAGCCCAGGTTGGCCTCAAACTTGCTATGTATCTGGAGACAACCTTGAACCCCTGATCCTGCATG
ACAGACATGTGCCCATAACCAGCTTATGTGGTGCTGGGATTGGAATGAAGGCTTTGATGGTTCCACGCCTGCTAGGCAGTATTCTACCAACTGA
TTTACATCCCCAGCCCCAAAGAATTCCCCAAAGAATTATCGGGACAACTTCAAGTCTTTCCTTCTCTTATGGCTGCACTGGAGACTTCCCACCT
TGTTAAAGAGCCAGTGCTGTTCCCCTGGAATGTGAGTGTATGCCCCCGCGCTACTGGACTTTCGCTGTTTCTTTTTGAGTTTTAAGGTGCACAT
GAGATACCTCCAATGCCCTTTAAAGTCCCTAGTCTTAGCTGGGCGTGGTGGCGCACGCCTTTAATCCCAGCGCTTGGGAGGCAGAGGCAGGCAG
ATTTCTGGAAACGCCCTACATAGACCAGAATGCTCCACCCCTGCCCCGCACCCTGTGTGGACAAGAATGTTCCACCACCTGCACACACACACAC
ACACACACACACACACCCTTTTAAATTTTGACACAGGATACTATGTATCCCTGACTGTCTGGGAACTCACTATGAAGTTATGATGGGCCTGGAC
CTCTCAGAGGTCCGCTGCGCCTCTTCCTTGAGCCCTGTGATTAATAAACATGTGCATTACCACGCCTGTGCCACCAAAGTCCCTACCATTTTTA
ACATTTATTTTCAAAGGTCACTTTGCAGCAATTTATTGGGATTAAACAAACGGTTCCTGATTCCAACTGAACATCTTATTTTTGTAATTTTAGG
GAGAGGTGTCTCCTCTAAACCCCTGGCCCACAGGGCTCTAGAGTGGATGCCTTGAAGTAACACATATGGTGTAATCTCTGCTTCTGAGCCCTGC
TGGGGGAAAGGGCAGCCCTGCTTCCTGTGTCACCCTAGAGTATAGCTAACTGCATGCCTGGGAAGTTGCCAGGTCATGTGGGATGGGCAGCTGT
CTGCATGGTGTCATTGGTGGGTTTGAGGAAATCCCCCTGCATAAAAATATCTTCTCCCGTCCAGGGCCTTGGCACTTACCCTAAATGTATCTCT
TACCATTGGTTGCAAGGTGATGTGGGAGGCCAATTGTTTACCAAGTTTGGCCAGAGAGTCCCACTAGCTCTCAGCGATGCTTCTTGCATCCAAA
TTCACCAGCCAGGATGAAGTGTACAGGAGACAGTTCCTTTTAATTAGGGACTTGATTCTTCAATGTGAGAAATCTGGGGTGATGGCCACTCAGG
GGAAGGACCGTGACGAGGACTCTTAAGTTACCTTCTGACTGATCACAAGACCAGTCCAGGAGATGGAAAGAGACGACTGCTTATGTTCCCAAGA
GAATTCCAGGCTGCCATGATGGCCTGGGAGTCTTTAATCCCAGCACTCAGGAGACAGAGGCAGGTAGGTCTCTGTCTCTATAAGGTCAAGGCTA
GCCTATAGTGAGTTCCAGGGTTGTAGTGAGACTTTATCTCAAAACATACAAACAAGCCGGGCAGTGGTGGCGCATACCTTTAATCCCAGCATTT
GGGAGGCAGAGACAGGCGGATTTCTGAGTTCAAGGCCAGCCTGGTCTACAGAGTGAGTTCCAGGACAGCCAGGGCTACACAGAGAAACCCTGTC
TGGAAAAAAGAAAAACAAAACAAAACCGAAACAAACAAACAAAAAACTATACAAACAAAAATAAAACACACATAAAAATAAGTAAAAAACAAAA
ACAAAAAACAAACTCACCACAGAACCCTAGGCGTGCTGAACAGGTGTAAGAGTGTCAGCCAAAACCTTACTTGAGGTGCCACAGCGAACAGGTC
GGTTAATTACCAGGCTAAGTCACCATGTCCCAGGTACCTGTGATGTCCTTGGCAATTACAGAATTGTGTCTAGGAGGGAGGGAGCTATTATTAG
CCTCAAATTACAGGTAAGGAAACTGAGTCTTCAGAGAGGTTCAGCTTGCTCAACCTCCAAATGGCTGAGTCAGGACAGAGGTAAAGGCAGCACA
CATGGGAAACCTGGCACAGCCTCCTGCTCCAGGGCTGGACAGACAACTATGACTTACCCCGAGTCATCTCAAAAGGAAAGGACCTTGTCCTGCC
ATAGCCTTGATGGGGCCTGGATCAGAGTCAGGTTTGGGTTTTGTTTTGTTATTTTTCTCTCTAAGGCCTTGCGTGTGCTTGAGCAAGTGATCTT
CCTTTGAATATAGAACCGTCTCTCATTTAAAAAAAAAAAAAAGTATTTTCAACTGAACAGGCAGGGGTGCTGCACACCTTCAGTCCCAGCATTC
AGAAGCAGAGGCAGGCAGATCTCCCAGTTCAAGACTAGCCTGGTCGACAGAGCGAGTTCTAGCCTGGTCTACAGAGCGAATTCTAGCCTGGTCG
ACAGAGCGAGTTTCTAGCCTGGTCTACAGAGCGAGTTCTAAGACAGCCAGGGCTAAGCAGAAAAACCATGTCTCACAAAGCCAAATAAAATANN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNACCTTCTTAGACAAAAGGGTCTCACTGTGTAGACCTGACTGTCCTGGAACTCCTATGTAGACTAGGATGGCCTTGAACTCACAGAGATCT
AAGAGTCTATGCCTCTCTCTGCTGGGATTAAAGGCATTCCATCCTATGCCTAGTCCAGGACCAGTGTATCAGCCTGCCCTATGCTCTATTAGTG
CTTCATCATGGAGGCAGATCTCAGCCCTGGCTTCCTGTGATGCTCAGGTGCCATTTCTCCTGGGCACAACAGTCTGCCTGTTATCCCACGTCCT
GGTTCTCTTGAAGATGGGCCTTTACTGTCTGTGACCTGAGTGTGCTTTCTAACAGACAGCAACACCCCAGGCATCTCCCAGGAATGAATGATGG
CTTGCATCTAAGTCACCTGTGGTCCTTGGGCAAGTTATTTCATTTTCTAGGTATGCTAGCAATTAAGAGAATTAGAGAAAAATATGCACATATG
GGCCATATGGATATACATGAATATGTATGTGCCATATGTGCCGTAGAAAAGGATTAATTAACACACAGCTAATATTAGATAGCAGTTGGACACA
GCCATGTATACCTGTAATCCAATCACAGTGACAGGGGAGGCTAAGGAAGGAGGCCTGCATGCAGTAGCTTCAAGCTCAGCCTGGTCTACATAGT
GACCCCCAGGCCAGCTAGGAATCAGGTGAAACCCTGTTTCAACTGCTCACCCTCCGAAAAGCTAGATTTTATGTGTTTTATTTGTTTGCAATGG
TAAGACAGGGTCTCATGTTGTTCAGGCTGTGGTTGAACTCCCTTTGTAGCCCTAGATAATCTTTTTTTTTAAATTGTCTTTATTTGTTTGTTTT
CGAGACAAGGTTTCTCTGTGTAGCCCTGGCTGTCCTGGAACTCACTTTGTAGACCAGGCTGGCCTGGAACTCAGAAATCTGCCTGCCTCTGTCC
CCCAAGTGCTGGGCTTAAAGGCGTGCACCACTGCTGTTCAGCTAGCCAAAGATAATCTTAACCCCCACTTCTCCTGCCTCCACCTCTGAGGCCT
GGGGTGACACTCAGGTTCCACCCTGCCTGGCATAGATATTATTCTTCATTGGATCACACCTCACACCTTCTTCACTGGGCCTCTGCTTTAGGAA
ACTGGTTATCTTTTTATTCATTTATTTATAGCTATTTATTTCTAGTGTTGGGGAAAGAATCCAGGACTTTGGAAATGTTATGGAAGAGCTCTCT
GCCACTGAGATCGGCCCCCAGGCTGGAATCTGGAAATCAATGCAAATCTCTCTCTTTCTCTGTCTGTCTCTGTCTCTGTCTCTGTCTCTGTCTC
TGTCTGTCTGTCTGTCTCTCTCTCTCTCTCTCTTGTTTGTAGACCAGGCTATTGAACTCAGATTCACCTGCCTCTGCCTCCGGAGTGCTGGGAT
CGAACGTAAGAGCCTTCACATCTGGCGAAGCACCTTCTTGAAGAAGGCAAAATTTTGCTTCAAAAATTTGTTTTTGGTGCCAAGAAGTGAACTA
GGCAAGATCTCTCACCAAGCTACACACCCAGCCCCGGAATTTGCTTGTTTTATTTTACTTTTTTGAGATAGAATATCAATATATAGTTCAATGT
GGCCTCCGATCAGAGGTCTTCCTGCCTTGTCCTCACTTCCTTCCTTTTTCAGTCATCGGAGTCCATGGGGTTAGAATCGTCCTGGCCACCTCTG
CCTCGACGTTTGTCTTTTTATGAGACTGATCAATTACGTTTGTACCAGTGAGGCACGTATGTTGTGGCTGTGTTGTGGAAAAGCAAACAGCCAT
CTGTCTTTTCATTATAATTTATCAAGACGGTGACTGGGACAGAGGGAGAGCCAGGGAGCAACCACATCGTCTCGTGTGTGTGGCCTCAGATCAC
ATTTGATTCTTTTTTTAGAGTTGGCAACACAGTTCAGCGGGTAAAAGTCCTACCGCCATGCCCACCAGCCTCAGCTTAGACCCCAGGACCTGTG
TGGTGGAAGGAGAGAGCTGACCTTCTGCAAGCTGCCCTTTGACCTTTATGCATACATTAGAGAGAGAGGAAAGCATCTGAGCTGCTCATGGGCT
CTACATACACAACAGGCCTGCCTCAGCCTTCCTAACAGCTGCTAGTAGGTGTGTGCCACCTCTAGTGATGCTTGCTGGCTTTTTCCTATTTACT
AATTGGTTATTTATATTTACATACATGTGCCTGTGTGTGCACCTACCCTACATGTGGAGGCCACACACACACACACACACACTCTTGGTGCTGG
AATTATAAGCACCAGGCCCGCACCCAGCTTTTATGTAATGTTCTCATGGTACAGAACTCAGGCTCTCATTCTTGCTTCGACACCACTTTGCCAA
CTGCATCATCTCCCTCGCCACCCCCATTTTTATTTTTTTGAGACCAGTTTTCCAGACTGGTCTCGAAATCAAGATCCTCTAGGGTCAATATCCT
GAGTGCTAGAATTACAGATATCAGCATCCCATATTGACTTAAATAATGTGGCGGTTTAAATAGTAAATATGGGCCGGGTGGTTGGGCACGCCTT
TGAACTGAACACTTGGGAGTCAGAGGCAAGTGGAATTCTGCGAGTGTGAGACCAGACTGGTCTACAGAGTGAGTTCCAGAATAGCCAGGGCTAC
ACAGAGAAACCCTGTCTCGAAAAAAGGAAAAACAAAACAACAACAACAACAACAACAACAACAAACTAGTGGCCAGGAAGAAGAGAGAAAGATC
AGGTGGCCTCTCCAGTTTGGAAAGAGACAGGATGAACTCAGGACTAGCTGTTTGCCAGCTCGGCTTCCATTCATAAGAAGGGTTTCAGGTGCGG
CTCTGACCTATAAAAGGATGACCTTCTGACCTAGTTAGTGTGTGAGTGTCTTTTTCTGGTCTTTACTACTTAGCTCAGGCTGCCCTAGAACGGC
CCCATTTCTCCCGCTTCAGCGTGTGCCACGGTGCCCCAGCCAAGTGGTTCCTTAGTAAAGTGGCACTGTTGGACACCTAGGCAAAGAAAGGTGA
TACCAGTGCTGTGTGTGGGCAGCACAGCTCGCTAAGTTCCCGGGATCAGCCAGTTTTCTATGTAGACACTTGGGGACTCTCACTTCTGAGCACC
TGGCACATACTGTTGAGGTCTGTAGTCTCAGGGAGCTGAGGCAGGTGGATGATTCCATGAGAATGAAGGGTTTGAATCCCATCTCAAAAATGTA
GCAAAGCTAAGACTCCAGTCTAAGTCCATGTTTAATAACAAAAAGAAGATTGGGGGGCTGGAGAGACAACTCAGCAGTTAAGAGCACTGGCAGC
TCTTCGGAGGACCCACGTTTGATTCTCAGCACCCATGTGGGGGCTCACAACCATATGTAACTCCAGCTCCAGTCTCAGAGGACCTGGCGCCCTC
CTGTGGCCTTCACAGGCACTACGTACACACGTGCTATACATACATACATACATACATACATACATACATACATAGCACGCAGACACAGAAATTA
AATTAAATGGAGCTGGGCATAGTGAAGAAACTAAAGCAGAAAGATCCCTTTAAGATGGAAGTGTCTATCTCCGAGCTAGAGTGAGTTTTAAGCC
ATGCTGGGCTACAGTTAGATTCTGTGGAAACAAATAACATCAACCAGAGCAATAACAATAATAACGAGCATAGAGAGAAAACAAAGCAAGCCAG
GTAGGGTGTACTTCAGCGGTTAGGCAGAAGANNNNNNNNNNNNNNNNNNNNCCTGGGCGCGCGGGTCGTTGGGACGCCGGGAGACCCCCGCGTG
GGTCGAGCGCGCTGAGTGTCCCGTGCCTCTTTCCTAGGCAAAGAGAAGGAGCCCCCTGGAGTCGCAGTACCAGGTGGGCCCGCTGTTGGGCAGC
GGTGGCTTCGGCTCGGTCTACTCTGGCATCCGCGTCGCCGACAACTTGCCGGTGAGTGGGCAACGCCGCCTCGGGGAGGGCGCGCGGAGGGATG
CTTTGAGCAGGAGGGTCTGATGGAGACTCGGGGGTCTTCTTCCAGGTGGCCATTAAGCACGTGGAGAAGGACCGGATTTCCGATTGGGGAGAAC
TGGTGAGTGAGCCGGAGCCAGAGACCCTCTCATGGGTGGGTGGGTGGCGACGGGCGTGCCGGAACCGGCGCGCTAACCCTCGCCCTCACTCTGC
AGCCCAATGGCACCCGAGTGCCCATGGAAGTGGTCCTGTTGAAGAAGGTGAGCTCGGACTTCTCGGGCGTCATTAGACTTCTGGACTGGTTCGA
GAGGCCCGATAGTTTCGTGCTGATCCTGGAGAGGCCCGAACCGGTGCAAGACCTCTTCGACTTTATCACCGAACGAGGAGCCCTACAGGAGGAC
CTGGCCCGAGGATTCTTCTGGCAGGTGCTGGAGGCCGTGCGGCATTGCCACAACTGCGGGGTTCTCCACCGCGACATCAAGGACGAGAACATCT
TAATCGACCTGAGCCGCGGCGAAATCAAACTCATCGACTTCGGGTCGGGGGCGCTGCTCAAGGACACAGTCTACACGGACTTTGATGGTGAGCC
AGGCCCCGGGGAGGGAGCTGCCCAAGGTGCCGGCCTGGAAGCCTCTGGCGGCCTCGGCTCCGGCCCTTTGTAAAGGTCATTGGGCCGCCTGGCT
CAGAGCTGGCAGGGGTGGGGCAGAGTCGAGCTTCCCGGCATAGTACAGCCGGGGATTTCGAGCTGGTTGAACGAACCTGCAACATTTCTGGCGT
GATTGGGGTTTTGTTGTTGTTGTTGTTGTTGTTTTTGTTGTTGCTGCTGGTGTTTGCTTGTTTTTCTTTTCAGCAGGAATTTATTAAATTCCAT
GCTTTCCCGGTGAATAAGAGGTCGTGGGTAGCCGGCTGTAGCCCAGATTTCTAAAGTATGACCCACTTTTCCCTCCAGTAGGAGGTGGAGCCGT
TGGTGGCGGGAGACATTGGTGCTGATTTTTTTCTTTTTCTTTTTTTTTTTTCTTTTGTAGTTTTGTTTTGTGGGGGAGTTTTAAGGTAGTTCTT
AGAACTGCAGGGTTTTGTTTTGTTTTGTTTTGCTTTTGAGGGTAAAGGAATAAGAGCCGCGGTTTCACCCCACTTGGGGGTGTTGTGGGGTGAG
GAGGCCTGGAATGAATGTTGTGAAATAAGATCTGTTGCTGGTTTGAAAATTAGTTGGGAGTCTCCGCTGGGAGGATGAAAACCTATCTACCATA
GGGAGGGGCTTGGATCGCGTTCTTTCAGCAGTGGAGGGCAGGAATAGTAGATCAAAGCGCGAGGGTGGGTCACGATCCGAGTGGCTCACCCGGA
CAAACTATGGCCTTTCAAAACTTGTGACTCTGGCTTGTGGTTTGAATTTCTTTGGAGGATGCCGGGCTGGGCTGCGTTTTTTGTATTTAACAGT
TAATGGCTGGCTGAGTCCTCCCCTGTGTTTTCCCCCCCTGGGCAAGTCTTTGCTGCCCCCTGTTGAGCACCACGAGGCTAGCACCTTTTTTTTT
TTTTTTTTTTTTTACTCCCCAAAGTTTGTAAACAGGAATCACGTGGTCTGAAACCAGCCCTGCAACTGTACCTTTCCAGTCTAGGGAGTGGGAG
AGTGCAGTGTCTATGCCTTGCTTGCATAGAACCCGGAGGAAAGCACACCCTGAGTTAGTTGTGTATAAAGGCCCTTACCTGGGCCCGGTCTAGA
TGAGACTGGTGTGGCAGGGCCCTGGGGTCCTTGTAGTTAAAGGCTAACCCACTGAGTGGGGCCTCTAGACTTGAGCTTTTCGGTGGCTTTATTT
GTCTTTGGGCCGATGTGTAAGGGATTTGGGTTTAGGGTTTTTGGGCTCCCAGAGAGCTACCATATCATTTCTTCATTTTCTGTCTGGACCAGAG
AGGCAGAGATGGAGAGAAAAAAAAAATATGGTTGTTTTAAAGTCAAATCTGCCTGTCTGCCTCCTGAGAGCTGGGATTAAAGGCATGTGCCACC
AAACCCGAGCCAGGCAGTGTCTTTATTAAAAAATATTTGAAAACCTAGTGGAGGTGGTGGTTCTTGCTTTTAATCCAGCCCTTGGGAGGTAGAG
GTAGGCAGATCTCCGAGTTCAAGGCCAGCCTGGCCTACAGAGTCACTTCCAGAGCTACACAGAGAAACTTTGGAGGAGAAAAAAAAAATTAAAA
GAAGCGGCTCAGACTAGCTGCTTTGTTACCTTTTTTTTTGCTAATGCTGGTTTCTTTCTATTCCCTCTGCTGGTAGGGACCCGAGTGTACAGTC
CTCCAGAGTGGATTCGCTACCATCGCTACCACGGCAGGTCGGCAGCTGTCTGGTCCCTTGGGATCCTGCTCTATGACATGGTCTGCGGAGATAT
TCCGTTTGAGCACGATGAAGAGATCATCAAGGGCCAAGTGTTCTTCAGGCAAACTGTCTCTTCAGGTAACTTCCCTCAGACACCCCCAATCCTT
CCCAAACCCTGGTTCAGGGTTGGTTTGGGGATTACAAGAGTGAGAGAGTATTCCTATCCACCTTTTTATTTTTATTTATTCATTTTCTTTGGGG
GAGAGTAAGGCCTTAGCCTGCCTCTGACCCCCAACTGCTGGGACTCAAGAGATGGTGGGTTTTCACTGGGTCAGAGTACTCCATTTGGGCTCAA
ATTGGAACAAGTTTTATATACATCCCACCCCAACCCCCAGACCCTGTAAAATCTAAGTACCAGCAGAGACCCAGTCTCTAAACAAAGTTTAAAA
AAAAAAAAAAAAAATGAAGGTTCCGGATAGTGTCAGTGGAAGGCGTGAGGTAGGTAGGTGAATTGAATCACCTCATCTTGCTCACTGGTGGCTC
ATCAAAACCCTTGTCATCCTTCCTATTTCTAGTGAGTGGGTGTCATGGGAAAGGCCCGAGCTATTGAAGTCTGAAACCACAGGTTTAAGAGGGG
AGGCACTTTTTTAACTGAAAGCTGATTGCTGGAGGCTTTCGGTATCCGTTCTTTCCCACCGGGCAGTGTTTGGTATAAAAAGTACCGTGCCTCT
TTAAAAACCCCAGACTTGTAGGTCTGGGGAAGAAGACCTTTGCATTACAAAAACAAGTTGAGTCATCTTTAACCTCTGTCTGCCCCGCCTTCTG
CAGAGTGTCAGCACCTTATTAAATGGTGCCTGTCCCTGAGACCGTCAGATCGGCCCTCCTTTGAAGAAATCCGGAACCATCCGTGGATGCAGGG
TGACCTCCTGCCCCAGGCAGCTTCTGAGATCCATCTGCACAGTCTGTCACCGGGATCCAGCAAGTAGCAGCCTTTCTGCTGCTGTCTGTCAGAC
CCTCCAGGGAGGGAGAGCTTGTCTTCTGGCCTCCAACAGGACCCTGCGTGACGATGCAGGGACAGCAATGACAACTCATTCCAGACTCCAGGTC
CCTGGAGGAGCCTCCCACAAGGGAAAGAGACTACTTCACTGGTCCTGGGCCCCTCTTTGCGCGCCCCGCCCCCAGACTCAGCGTCTAGTGTTGC
TGGGCTCCCCTGAATCCATCCCTGGTTGGGGGTGGGAGTGGATCAGAGCCCTGTCAATGGAACTTTAATCACCATGGAGACTGGGTCACCAAGA
TGGGCCAGGGGGGAGGGGAAAACAGTCTGGGGGGGTGGGATAAAAACTAGCACCATATTTAAGTCCCTGTCACCTCTTCCAACTCCTGAGTGCC
TTCTGTGGGGACTCCGCCTGTGCTGGGAGAAATACTTGAACTTTACCTCTTACCTGCTGCTTCTCAAACTCTGCCTGGGTTTTGCTTCCTTCCT
CTCCTCTCCTTTCCCATCCCCCACTTCCTATGAAAGGTGCCATGGGAGAGGCTACAGGGCCAACTGCTGAGCCACCTGCCCCTTTTCTGTCTCC
TTTAGTAAAATTCCCAAGCAAACTGGTCTTCCTTTTTTGTTTCGACTTAACTGTTTCAAAACCAAGACCTCACACAGAACGCACAACCAATGCA
AAAAAAAAAAAAAAAAAAAAAAAAAAAACCAGACAAGCTGTAAATGTGTGTATAGTTCCGGCATGGTAGCCTGCAAAAGGACTGTAGATGATAA
CAATTTTAAAGAAGAAAAAAAAATGCCTTTAAGTTATTTTATGTGTTGGGGGGTTTGGTTTGGTTTTTTTTTTTTTTTTTTTTTCTTTTTTATT
CTTTTTTGCTTGTTTTTGAAAGATGGCACGTTCTACCCCGGAAGTTTTAATGTATTTATTTATTTATTTATTTGGTTGCCTTCCTGTCCAAGCT
TCCACTGACTGTTGCCCCCGGTTTATTGTCATGTTCTGCCTTTTTTTTTTCTTTCCTTCCTCCTGACTTGGGGACCTTTTAGGGGAGGGCTGTG
ACGCTTGCTGGGGTGTTGGGGCTCCGGTGGGACAGCTGCTGTAGCCCCCTTCTCCAGGGGCCCCCTTGGCTGCTTGCTGGAGTGGGTCTAGGGT
CTGCTGACTGTACATAAGACAGTTCCGGTGTGGTGTGCCTTCCGGATCCTCTCTGGGGCTGTGCTTTGAGCAGCGTGCAGCCCGCTGGTTTTAT
CTGAGTAAAGTATACTGTACAGGGGAATAAAAGAGATCTTATTTTTTTTTTTTATACTTGGCATTTTTTGAATAAAAACCTTTGTCTTAAACCT
TGAGTTTTCCATTCATTCATACAAAGNNNNNNNNNNNNNNNNNNNNCTGACGTGTCATGTCAGAGCTAGCATCGAACACTTGAGTTCCATCAGA
CAATGTGGTTGCAGGAATTACTACAGGTAGCACTTGAGAGTTTGATTTGGTTTTTGTTTTAAAGATTTTATGTATGTGAGTACACTGTAGCTGT
CATCAGAACAGGGCATCAGGTCCCATCACAGATGGCTGTGAGCCACCATGGGGTTGCTGGGAATTGAACTCAGGACCTCTGGAAGAAGCAGTCA
GTGCTCTTAACCATTGAGCCATCTCTCCAGCACCCTGGTTCTTGGTACCTCATAGCAGGGTTTCTCTTGAGCTCTAGCTGTAACTAGGGCTGTA
GATGAGGCTGGCTTCAAGCTCAGACCTACCTGCCCGGCTTGGTGAGTGACCAGAAGCCTAGTGTTTTTGACACAAGGTTTGAAGTCTGTGTAGC
CTTGGAACTTGATATGTATAGACCAGGCTGGCCTGAAACTCCTGGATCTGCCTGCCTTTGCCTCCTAAGTGCTGGGATTAAGGGCATGTATAGT
TCCACTGGAAAAGATTTGGGAGCCACGAGTCCTAGCATCTTCCTAGCAGAGTGGTGTAATCCTATAATCCCAGCACTTGGGAACTAAGAGGATG
AGAGTCAGGAGTTCAAGGTCAGTCTCAGTCTGGGATACTCGTGACTGGTCTAATCAGCTGGAAAGATGCACTCTGGTAGACAGGACCAAAGGAA
TTCCTATACATTCAAGGCTGAGCCTGGGCTGGAGTTCCAGGTCTATCCTATCTCGATAAAAGGGGATGAGGATGGGCAGGGGAAGTGGAGAGAT
GGCTCAGCAGTTAAGAATTCTTGCAGAGGGCCTGGGTACAACTTCCAGCACCCACATGGTGGCTCACAACCATCTGTAACTTCAGTTCCAGAGG
ATCCAATGCGCTCTTCTGGCCTCCAAGGGCACTAAGCGTGCATTCATACAAATACACAGATAACACACCTATTAAGAAAACATTTCAGCCGGGC
AGTGGTGGCGCATGCCTTTAATCCCAGCACTCGGGAGGCAGAGGCAGGCAGATTTCTGAGTTCAAGGCCAGCCTGGTCTACAGAGTGAGTTCCA
GGACAGTCAGGGCTACACAGAGAAACCCTGTCTCAAAAAACCAAACAAGAAAGAAAGAAAGAAGAAAGGAATAAAGAAAGGAAGAAAGAAAGGA
AGAAAACGTTTGCTGTTCTTGCAGGGGACCCAGAATTGGTTCCCAGCACCTGTATGACCTCACAATTGTCTGGAAGATGGGGCCCCCAGTTTGC
CTCATGTTTGGTCTGAGTATGTCCTAATAGTTGAACCACTGCTGATCCAGGTCCTAAAAGTAATTCTTTCTTTCTTTCTTTCTTTCTTTCTTTC
TTTCTTTCTTTCTTTCTTTCTTTCTTTCTTCCTTCCTTCCTTCCTTCCTTTCTTTCTTTCTTTCTTTCTTTCTTTTTTTTTTTTTTAGGCAATT
TCTCCTGTAGCCTAGGCTTAAATAGCCCAGGAGAGCCTTGAACTCCCGATCTGTATCTAGGATCTGGGGCTTGCTGCCACCACACCCAGGTGTA
AACATTGCTTTCTTGTTGCTTTCTGCCCATTAGTAACAGTTCTAACATTCTTCCCTCCTGCAGGGGCATCTTGACCTCTTCCTCCCCTCTTGCC
ATAATCTCTTAGGAACTTGGCCCCATTGCATTGCGCTTGTGGAGGATCCAGGCCACAGTGCCCTCCCTTCATCGAGAGATCTCCTCTACCACCA
CATCTTGGACTAGGTCCCAAACCCCAGCACAGCCTCGGTGTGGCTCAGAAAGGAAGGAAGGATTTCTCAGCTCTTGGGGGCTGATTTTCTGAAG
TTCCCCTTTTCCCTACCCTTCTGTCCCCTCCTCCTTCCTGTATACACACTGCCCGGCAGCTGGGTGTCTAAGCACACTGTGGGACCTTTCCAGT
CAAGTTGCAAGTGAACAATTGAAAGTTGCTAGAAACTTTATTTATTTATTTCATGTATGTGAGTACACTGTAGCTGTCTTCAGACACACCAGAA
GGGTGCATCAGATTTCATTGCAGATGGTTGTGAGCCACCATGTGGTTGCTAGGAATTGAAATCCCCTGCTAGAAATGAAATCCGGCAGAGCCTT
GCCCTTCTTGCCTTTGAGATGAGTTGGGGGGGGCCTTTCTGTGGCTGTTGAGACTGCTGGGGGCCATACAGTGTGTCTGTAGAAAGTAGGTTCT
CAGGGGCTAGAGAGAGGGCTCAGTGGTTAAGAGCACCGACTGCTTTTCCAGAGGTCCTGAGTTCAATTCCCAGCAACCACATGGTGGCTCACAA
CCATCTGTAATGGGATCTGATGCCCTTTTCTGGTGTGTCTGAAAATAGCAACAGTGTACTCATATACATGAAGTAAATAAATAAATCTTAAAAA
AAAAAGAAAGAAAGAAAGTAGGGTCCTCGCTAAGTAGAAGAGGTCAGAGGTTGTAGGAGATGCTTTTAGGTTCTGTTTGTTTTGTTTGTTTTTT
GTTCTTTATGTAAACCAGGTTGGTCTTAAAGTCACAGAGATCTACTTGCCTTTGCCTTCCTGAGTGCTAGGGTTAAAGGTCTGTGCCACCACAC
CCTGCATTATTTATTTTTTATTTTATTTTAATTTAATTTAATTTTATTTTTTATTTTGAAATAGCATCTCCTGCATTCTAAGCTAGACTTGAAC
CCTGGCATGATAGGTGTGTGTGAGCTGAAACTAGGCATTTTTTGAGAAGGGGAAAGGTCCTTGATCTCTCTCTCTCTCTCTCTCTCTCTCTTTT
TTTTTTTTTTTTTTTTTTCCAGACAGGGTTTCTCTGTGTAGCCCTGGCTGTCCTGGAACTCACTTTGTAGATCAGGCTGGCCTCGAACTCAGAA
ATCTGCCTGCCTCTGCCTCCCGAGTGCTGGGATTAAAAGCATGTGCCACCACCGCCCGGCGTGGTTGTCACTTCTAAGGAGTAAGAAGAGGGTT
GTGATCTGGGGAGAGGGACCGGCCCAGCTATCTTCGCATTAGTTCCCATCCAAGAGATGGAGACAGATCTGTTTATGTCCCTGTTGATTTGCTT
TTTAGCTGTATGACATGTTGTGACATTATGGACAGACCTGTGTTTCCAGGAGGCAGAGATGGGGATGGATGTCCTTGGCGGAAGAGATTGGGAA
ATCTCCCCAGGGAAGGCAGCTTCTCACTGTTTTGTTTTGTTTTGAGTCAGGGTCTCCATACATGTAGCATGGGCTGGGTTCTGATGCTATGGTG
CCCTGGGAGCTCAGAAAACAACTTGNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNGCCGGGCACGTTTCTCGTAAGTGTTGCTCAGCTCCTTTAACTGCTCCGCT
GGTTCTTGGAAACCCTCACCACAAGGACCTATCGAGAGCAGTGTTGGAGCAGATATGGCATAGCCCTCCCCCCCCAACCCCCCCCAGTTCATTT
CCGCAATGGCCTGTGCTACTAACCCCGATTGCCAACAGCGAGGTTGCAGTGACCCCTCCAGTGGTGGTGTGAGGGAGTGGAAACGAAGCAGAAC
TCAGGCCAAGATGAGGAGACCCCGTATTATTTTAATTTACAGTAATAGGACTTCCAATGTGCAGAATTCTTGGTGACCTTAGGTCTGGGATACA
GTACAAGACCGGCTGTTATTGAATTGTATCCTGTTGTTTGGCGAGTCTAACACCAATAGCAAGCAAGTTGGTGGTGTGGCTCAGTGGGTAGAAC
GCTTACCAGGCATGCACGAAGCCCTGGGTAGGGTGTTTTTTTTTTGTTTTGTTTTGTTTTTTTACTTATTATATGTAAGTACACTGTAGCTGTC
TTCAGACACACCAGAAGAGGGCATCCGATTTCATTACAGGTGGTTGTGAGCCACCATGTGGTTGCTGGGATTTTGAACTCAGGACCTCCGGAAG
AGCAGTCAGTGCTCTTAACCCCTGAGCCATCTCTCCAGCCCCCTGGGTAGGGTTTGTAATCCCTGTGCTGGGGAGGTAGAAGCAGGAGGCCCAG
ATGTTCAAGGTCATGCTCAGCCTGCATTATAGGCCACCCTGGACTACAGGCAGGACAGTAACAGTCACGTCTATGTGACCCGATGGTTTTCTTA
GTTTGTGTCCCGATGCCCCCAGATAGACCCTGCTGTCCTGAATGCCAGGTGTAGCTTTTTTTTTACCTTAAAATAAATGCTTTGCCAGGCAGTA
GTGGCCCACGCCTTTAATCCCAGCACTTGGGAGGCAGAGGCAGGTGGATTTCTGAGTTTGAGGCCAGCCTGGTCTACAGAGTGAGTTCCAGGAC
AGCCAGGGCTGCACAGAGTAACCCTGTTTTGAAAACAACAACAACAAAGAAATAAATGCTTTTACATTCATTTACTTTGATGTTGTTGGTTTTA
AGATTTATTTTCAGTTTGTAATTATTTGTGCACTTGTGTGTGTCTGTGCGGGGGTGTGCCTACAGTCGGGCACAAGTACTTGCAGAAATCAGAA
GGGGACACTGGACCCTCTGGAATTGGAGTTGCAGGCAGTTGTGAGCTGTCAGATCTGAATGGTGGGAACCAAACTTGGACCTTCTGTAAGACTG
GTATGTATCTTAAGCCCTGGACTATCTCTACTGACCCCATCCGTGATCATGATCGTGTCACCATCACCATCATCAGCACCACCATCATCATCAC
CATCTCTGGCTAGCCTTGAATTTTTTTTTTTTCTTTTGAGACAGGGTTTCTCTGTGTAGCCCTGACTGTCTTTGAACTCACTCTGTAGACCAAG
CTGGACTCAAATAGTGCTGGGGACAGAACCCAGGGTTCCTGCAATGCTAGGTAAACCCTCTTATCAACTGAGCCACATTGCCGCCAGCCCATGA
GTTTTGAATAAACAATTAATTTTGTGTTTTCCAAAGTTTTGTCATTGTATATAAAATACCCTTTTAATCTAACTCCCTGGAACCATGAGGGCCC
ACATGAAATCAGGGGCAGTAGCAGCGTGGTGTGTAAGGGACCCTTCCTTGACTTGGAGACATATTGTCACAGTTGAATTCCGCTCCAACTTTGG
TCTTCACGTGACCTTGGCAGATATTTAGTTTCTGAGTTTTGTTTTTTTTCTTCTCATCCACGACAGAGAAATGGTCCGGAGTTTGTGAAGCCTG
TGCCACCCAGCCAACGGTCATTGGTACAAATGAGGCACAGCTTAAACAGGACACCGTCGAAAGGAGATTTTAGAATGAGCTAGTCTGAATCTCG
GTGACCTGGAGCTAGTGGGGTCCAGAGATAGACTGAAAAGGGCGCCAGGAACTTCAGGGGAGGGAGTGCGGACTTGTTTTTCAAGTATGGTAAT
GTTTATTCGGCTGTGAAAGGCTGTCAGAAACACACCCTACTGAGCTAATGCTTAAAATGCATGCAGTGTCTTATGTGTATAAATTACCCCTGGG
TAAAATGACTTAAAAAAAAAAAAGTTAATTTCTTTTCCTTTTCCCCATCAATGAAAACACGTCCCTGAGCCAAATATACCACGGAAGTCTCCTT
TCCGGTTAGATTATTCATGTGAGTGCGAACATTCTCCCCGTGGATTGATATAAATAAATGAGAATTGGCTTAATTTCCCACCTTACTTTCTGAC
TGTAGTGCAAAATTAGAGAGATGTGTCATCAGGGGGTGGAGGGGGCCGACGCTGCGACCACGCTACGACGGAGATAAACTTAAACGCCGCATTT
TTTTTTTTCTTCAAAGTCATAAACTTAAAGGGAAAAGTTTTGTTTTTTGTTTTTTTTTTCCTGGCGAAGCACTAAAGCAAAGTGATGGTTGCTC
GCGGTGGCGGAACATGTCTGGGAACCTCGCACCTTGGGAGGTAGAGGCAGGAAGGTCAGGCAGCCAACACTTGGTGAGTTCCACGCCAGCCTGG
GCTATGTGAGACCCAGTTGCTAAACAGCAAAGCGAGATGGTGCGGTGCGGCACAGGCTGACAGCTCTGCAGTAGCCAAAGGAATCACGTGATCC
ATCTGGTTCTATCTCCCATCATCCCTCATTTCTTTCCCCTTTGGGGTGAAATGTACACATGTATCTTTGAAGGAATTTTTTTCTTTAATATCTA
CTATTAACATTTTTGCTTACAACTCCTTGGCTTTTATTGTCTCCACACCTAGTCAAAGAGAAACTAGACATTTTAATCTATTTTTCAGGTAGCC
ACGTAACTAGAGATGAGAAGATGAGAACGGATTTTGCTGTAGATGTCTATCCATCTCTGTGTCTTTGGGTTTTTTAAGATTTACTTTATTATAT
ATACACAGTGTTCTGCCTGCATGTATGCCTGCAGGCCAGAAGAGGGCGGCAGATCACCTTATAGATGGTTGTGAGCCACCATGTGGGTGCTGGG
GATTGAACTCAGGACCACTGGAAGAGCAGCCAGAGCGCTTAACCTCTGAGACATCTCTCCAGCGCAGTCCATCCATCTCTATGCATATGTAGGT
TAGGAGTAGTGCAAGGTCAGAGCTCAAGGTCCTAAGCAGGTGGTGCACACTTGTCAGCCTAACTCTAGGGAAAAGGAGGCACGAGAATCAGGAG
TTCAAGGTTAACCTTGGCTATGTAACAAGTCTGCAGCCAGTTTGGGCTCCAAGAGACCCTGTCTTGGTAAAATGAAGCTGATAAGCACACTTGC
TGCTCATCCAGAGGACCCAAGTTCAAGTCCCAGCACCCACACAAATTGGAGGGCAAATTGGTATTTAATTATTTACTATCTGTTTGTGCTATTG
GGATTTAGAACTGAGCTAGGCAAGTGCTCTACTCCTGAGCTCCACCCGTTGCTGCAGTCTTACATATCCAGGTGAGCACGTGGAGGTCAAAGGA
CAACTGGAGAATTGCTCAGAAGTAAAGAGCACTGGATGCTCTTCCAGAGGTCTCAAGTTCAATTCCCTCACCCATATAGTGGCTCACAACCATT
TGTAATGTGAGCCAGGTCTCTCCTGGCTCAGGGGTGCAGGGTGCAGGAGGTCTAACTTAGCCCTCTCGTTTACTTTGTGCCACAGGGTCAGTTG
TCCTTGTCACTTGGCTCTTCAAACAGTTACATCTTTTTGATCACCTGGGATTCCTTCAGGCTGGGTTCTGTGCTCTTTATTTTAAGAATATTGT
TATATTTTAAGGGGGTGGGCTGTGGATCAGGGGAGTGGGAGTCATTTCTTCTAATCATTTTGGTCCCGGGGATGTAATTCAGGTCCTCATCTCT
CTCTCTCTCTCTCTCTCTCTCTCTCTCTGTCTGTCTCTGTCTCTCTCTGTGTCTGTCTCTCTGTGTCTGTCTCTCTGTTTCTCTGTCTCTCTGT
TTCTCTGTCTCTCTCTCTTTGAGACAAGGTATCCCTGGGTGATGTGAAACTTGCTGTGTAAACCAGACTGGCCTCAAGCTCATAGGGATCACCT
GCCTTAGCTTCCCAGTGCTGGGGTTCAAGATGTACAAGGCCTGGCTGGAGAGATGGCTCAGTGGTTAAAAGCACTGACTGCTCTCCCAGAGGTC
CTGAGTTCAAGTCCCAGCAACCACATGGTGGCTCACAACCATCTGTAATGGGCTCTGATGCCCTCTTCTGGTGTGTCTGAAGACAGCTACAGTG
TACTCATAT
MOUSE SEQUENCE—mRNA
TCTAGAGTCGACCTGCAGCCCAAGCTTCCTTAATGGAATCTTCTGGTTTATTTATTTCTCACCTTTGGCCTTATTTTTTCCTGTGCTCACTTAA
TATCCTGGTTGCTGTTGTTGTTGTTGTTTTGTCTGCTTGGTTTTTTTTTTTTCTTCCCTTGTTGATCATGAACCCCAGGCATGCTAGATGACTA
TCTGATCATTGAGCTACATCCTCAGCCATAAGGCTTTTCCTTTTGACATATGGTCTTGGTTACACCACTGTCTGGCTTCACTATACTGCTCAGT
TTGGCAGTCCTCCTGTTTCAGCCTTCCAAATATTAGAATGACAAGGATGACCCACTACATCAGGTTCATGAATTGCTGTTAAGTGTTCTTTACG
TTGGGTCATCTCCCATCAGAAACGGTTGGATAAAGGGTGGTTGTGCTTGCAGTGTGGGCAAGAGGGAAGGCAGAAGCAAACGATCCATGAATTT
GAGAACTGTCCTGGCTTTGGCATGCACAGTGAGATTGTATATTAAGAAGCAGAAAAAGGAAACAAAGCAGGTAGACAGTATAAATCAACGAAGA
GATAGCATACAGAAAGTCTTCTTGTTTTAAGGCAGGATCTTTTGTGTCTCTGACTGACTTTGAACTTTGACCTGACTCTACCTCTTTGTCCTGG
GATCAAAAGTACCATTTATGCAGTGCCCAGGGATCAAACCTAGGATTTTCTTCTGTATGCCAAGCAAGGACCCTACCAACCGAGCAGCATCCCC
AGCCCAGAAAGATTTTTTTTCTTTGTAAGAACCCCACATGACCATCAGATGGGCGGCAGTGAACGAAGGCTTAGAATTTGTTCAGCAAAAAAAT
GTGGAAATACACGCGCCTTGTTCAGAACGCAAGGCTTTGAATAAGCGACAGGACTGGAGTGTATGTAGGTTGGTGACAGGGTGCATGTGTAAGA
CCCTGGATTTCCCAACATCAGACACATTCCGAGAAGAGGACAGGGCACTTTGGACAGGTGGACTTAGTTTTCAGAGGCACAAAGTCTGGAATGT
GCTGGGAGATTGAAAACAAGTGGAAATAGTCCTGTTTGGACAGAGCTGAGGGCCTGATAGGGAACTGGAGGTAGTTGTACTGGTGTTTAACACA
TATTTATTTATAGACAGAGTTTCTCTTTGTAGACAAGGCTGGCCTGGTTAGAATGTTGTGGTTTTAACGTTGGGCACCACTCTAGGCATACCTG
TTTTCTGATTGCCTGCTCAATACCAGTGCCATAGATGTTTTTAGGCTGATTAAAGGGAGAGCTCTGAACCCTCAGGAGCAGAATTTGGGCCATT
CTCTCTGCTGTCGTATGACCCTGTGTCATCCCAGTGACGAGTCATTTTCCTATCTGCAAAAAAGGAAGTAATTCTCCTTCCCCTGGGTTTTTCT
CCTTTCGTCATCAGAAATGAATGAGTTAGTCCACAGAACACAAACAAAGGGAAATATGTAATGTTTTGTAAGAGGCATCCATAAAAGTTATGGA
GTCTCTGGGCATCTTTATAAGCAGCATGGAAAACTTGTAAAGGAAGCATTTAAACGCGTGCGCTAATGACTACATCCACAACAGGCTCGTTCCT
CCTTTAAAGATCTCTCAGACCTAAGAACAACAACAAAAATAACGAACAAGGGGTGTAGCTGGCTGTCCATGACTCTGGCCACCAGTATCTCAGT
TTATATGGATTACACAGAATGAGAGTTTACCCATTTCCACACGTCGTTATCAACTCTCCATGAAGTTCAAAAGGACAAAAGATGAAAGCGGACT
CGGCTAGCTTTCCGTGGTATTTCCACGTGCATGTGGGGACTTTCCTTTGAGACAAGGCTGGGGTGGGGGCCAATAATATCTGGTATCGCCTGAC
TGCTAATACCAAGCTGATGAGAAGGTAACTACCGCAACAACCTCTGATTAAACCCAGCGGCACCCTCCCACCCTCGCCCGCGATCAGGAATTTT
CCTGCTCCTCCCGCCCTCTCAGAAGGGGCTGAGGCAGCATTTGGCAAGAGAAAGCTAACGTTTGTGTCGTGATTTCCTCTCTCAGGCGCTCGGA
CATGGACCGAGGGGATCAGGTACCCCGGGTCCCTTCTAATTCTCTCTGCATTTCTAAATTGAGAATCTTTGTCACCGGCTCCTCGCATCCCAGG
TCTCCCCCACTTTTCCTCGCAGGCCTGTGACTGGCCTCGGTGTGCAAAATGCCGGTGACTCACGTCTTGCACAGCCTTCGCCCCCAAAAACACA
ATCATCACCCCCAGGATCTAGCCCACATAGCATCGTCAACCCGCCCCAGCAGCCTTGGAGGTCTCCGGGCTGAGTCACACCAGAGGACCACCCT
GGTCGGCATCCACACACACGCGGTCCCTACTCATGGCTTCCGCGCCCGCCACACCAACGCCCTTCCATTTCTCACAACTTGCTACGCTCCAGCT
CCAATCCTCGACCCCCCTCCACAACCCAAATCTCCGGGAAACACAGAGCCGGCGGAGCTGGCCTCGGGCGGCCTTTGATGGCTTTGTTATTGTT
TGGGTTTGAATCGATACCCCCTCCCCATCCTTCCACCCCCGCCCGCGCGCGCCACCCAGCTCCCGCCTCCCTGCACGCCCAGGCCCCTCCCCCT
CCGTTTTGGCGCCTTTTCCTTCCCGCCAGTCGGCTTAGACCATTCTGACCGCGAGGCGAGAGGCGGGGCCCGCAACGCCTTATGCAAATAGGGC
GTCTTCTGGTTGGCTGAAGCGCGCCCGAGGGCGGGGCCGGGGAGAGGGGTGTAGCCGCGAGGGGGCGGAGCGGAGGGGGAGGGCCCTGGTCCCG
CCGCCTCCCCGCCCCCTCTCCGCCCTCCGCGCCGAGCCAGCCCGGCTCCCCACCTCGCTCCCGGAGAGGCCCCGCCCCGTCCCCGCCCGCCGCC
GCCCTCCCCAGCGGCGCGTCCGCCCCTTTACTCCTGGCGGCGGGGCGAGCCGGGCGTCTGCAGCAGCGGCCGTGGCGGCGGAGGAGGCTGGAGG
GGAGTCGGCAGTGCCGGCGGCGGGATCGGCAGTAGCAGCAGCAGCAGCAGTCACAGTCAGCAGCAGCCCTGGGTCCCGCAGCGCCTCTCGCCCT
GCCGCCTCCCGCACTGCCTGACCCAGCCGGCGAACCCGCCCGCGGCCAGCCCCGCCAGCGCCCTCAGTCGTCCTCCGACTCGCCCTCGGCCTTG
CGCGCCAGCCGCAGCCAGAGCCGCAACGCCACCCGCAGTCTGAGTCCCGGCCGTCGCTCAGCCCCAGCTCGCTCCGCCGCCGCTGCTGCTCGTC
CCGGCACCGGCGCCGCACCGACACCCTGGAGGTGGGGATGCTCCTGTCCAAGATCAACTCCCTGGCCCACCTGCGCGCCCGCCCCTGCAACGAC
CTGCACGCCACCAAGCTGGCGCCGGGTGAGTGTCCCCACCCCACCCTGGGCGCGCGGGTCGTTGGGACGCCGGGAGACCCCCGCGTGGGTCGAG
CGCGCTGAGTGTCCCGTGCCTCTTTCCTAGGCAAAGAGAAGGAGCCCCTGGAGTCGCAGTACCAGGTGGGCCCGCTGTTGGGCAGCGGTGGCTT
CGGCTCGGTCTACTCTGGCATCCGCGTCGCCGACAACTTGCCGGTGAGTGGGCAACGCCGCCTCGGGGAGGGCGCGCGGAGGGATGCTTTGAGC
AGGAGGGTCTGATGGAGACTCGGGGGTCTTCTTCCAGGTGGCCATTAAGCACGTGGAGAAGGACCGGATTTCCGATTGGGGAGAACTGGTGAGT
GAGCCGGAGCCAGAGACCCTCTCATGGGTGGGTGGGTGGCGACGGGCGTGCCGGAACCGGCGCGCTAACCCTCGCCCTCACTCTGCAGCCCAAT
GGCACCCGAGTGCCCATGGAAGTGGTCCTGTTGAAGAAGGTGAGCTCGGACTTCTCGGGCGTCATTAGACTTCTGGACTGGTTCGAGAGGCCCG
ATAGTTTCGTGCTGATCCTGGAGAGGCCCGAACCGGTGCAAGACCTCTTCGACTTTATCACCGAACGAGGAGCCCTACAGGAGGACCTGGCCCG
AGGATTCTTCTGGCAGGTGCTGGAGGCCGTGCGGCATTGCCACAACTGCGGGGTTCTCCACCGCGACATCAAGGACGAGAACATCTTAATCGAC
CTGAGCCGCGGCGAAATCAAACTCATCGACTTCGGGTCGGGGGCGCTGCTCAAGGACACAGTCTACACGGACTTTGATGGTGAGCCAGGCCCCG
GGGAGGGAGCTGCCCAAGGTGCCGGCCTGGAAGCCTCTGGCGGCCTCGGCTCCGGCCCTTTGTAAAGGTCATTGGGCCGCCTGGCTCAGAGCTG
GCAGGGGTGGGGCAGAGGCGAGCTTCCCGGCATAGTACAGCCGGGGATTTCGAGCTGGTTGAACGAACCTGCAACATTTCTGGCGTGATTGGGG
TTTTGTTGTTGTTGTTGTTGTTTTTGTTGTTGCTGCTGGTGTTTGCTTGTTTTTCTTTTCAGCAGGAATTTATTAAATTCCATGCTTTCCCGGT
GAATAAGAGGTCGTGGGTAGCCGGCTGTAGCCCAGATTTCTAAAGTATGACCCACTTTTCCCTCCAGTAGGAGGTGGAGCCGTTGGTGGCGGGA
GACATTGGTGCTGATTTTTTTCTTTTTCTTTTTCTTTTTTTTTTTTTTTTCTTTTGTAGTTTTGTTTTGTGGGGGAGTTTTAAGGTAGTTCTTA
GAACTGCAGGGTTTTGTTTTGTTTTGTTTTGCTTTTGAGGGTAAAGGAATAAGAGCCGCGGTTTCACCCCACTTGGGGGTGTTGTGGGGTGAGG
AGGCCTGGAATGAATGTTGTGAAATAAGATCTGTTGCTGGTTTGAAAATTAGTTGGGAGTCTCCGCTGGGAGGATGAAAACCTATCTACCATAG
GGAGGGGCTTGGATCGCGTTCTTTCAGCAGTGGAGGGCAGGAATAGTAGATCAAAGCGCGAGGGTGGGTCACGATCCGAGTGGCTCACCCGGAC
AAACTATGGCCTTTCAAAACTTGTGACTCTGGCTTGTGGTTTGAATTTCTTTGGAGGATGCCGGGCTGGGCTGCGTTTTTTGTATTTAACAGTT
AATGGCTGGCTGAGTCCTCCCCTGTGTTTTCCCCCCCTGGGCAAGTCTTTGCTGCCCCCTGTTGAGCACCACGAGGCTAGCACCTTTTTTTTTT
TTTTTTTTTTTTACTCCCCAAAGTTTGTAAACAGGAATCACGTGGTCTGAAACCAGCCCTGCAACTGTACCTTTCCAGTCTAGGGAGTGGGAGA
GTGCAGTGTCTATGCCTTGCTTGCATAGAACCCGGAGGAAAGCACACCCTGAGTTAGTTGTGTATAAAGGCCCTTACCTGGGCCCGGTCTAGAT
GAGACTGGTGTGGCAGGGCCTGGGGTCCTTGTAGTTAAAGGCTAACCCACTGAGTGGCCTCTAGACTTGAGCTTTTCGGTGGCTTTATTTGTCT
TTGGGCCGATGTGTAAGGGATTTGGGTTTAGGGTTTTTGGGCTCCCAGAGAGCTACCATATCATTTCTTCATTTTCTGTCTGGACCAGAGAGGC
AGAGATGGAGAGAAAAAAAAATATGGTTGTTTTAAAGTCAAATCTGCCTGTCTGCCTCCTGAGAGCTGGGATTAAAGGCATGTGCCACCAAACC
CGAGCCAGGCAGTGTCTTTATTAAAAAATATTTGAAAACCTAGTGGAGGTGGTGGTTCTTGCTTTTAATCCAGCCCTTGGGAGGTAGAGGTAGG
CAGATCTCCGAGTTCAAGGCCAGCCTGGCCTACAGAGTCACTTCCAGAGCTACACAGAGAAACTTTGGAGGAGAAAAAAAAAATTAAAAGAAGC
GGCTCAGACTAGCTGCTTTGTTAACTTTTTTTTTGCTAATGCTGGTTTCTTTCTATTCCCTCTGCTGGTAGGGACCCGAGTGTACAGTCCTCCA
GAGTGGATTCGCTACCATCGCTACCACGGCAGGTCGGCAGCTGTCTGGTCCCTTGGGATCCTGCTCTATGACATGGTCTGCGGAGATATTCCGT
TTGAGCACGATGAAGAGATCATCAAGGGCCAAGTGTTCTTCAGGCAAACTGTCTCTTCAGGTAACTTCCCTCAGACACCCCCAATCCTTCCCAA
ACCCTGGTTCAGGGTTGGTTTGGGGATTACAAGAGTGAGAGAGTATTCCTATCCACCTTTTTATTTTTATTTATTCATTTTCTTTGGGGGAGAG
TAAGGCCTTAGCCTGCCTCTGACCCCCAACTGCTGGGACTCAAGAGATGGTGGGTTTTCACTGGGTCAGAGTACTCCATTTGGGCTCAAATTGG
AACAAGTTTTATATACATCCCACCCCAACCCCCAGACCCTGTAAAATCTAAGTACCAGCAGAGACCCAGTCTCTAAACAAAGTTTAAAAAAAAA
AAAAAAAAATGAAGGTTCCGGATAGTGTCAGTGGAAGGCGTGAGGTAGGTAGGTGAATTGAATCACCTCATCTTGCTCACTGGTGGCTCATCAA
AACCCTTGTCATCCTTCCTATTTCTAGTGAGTGGGTGTCATGGGAAAGGCCCGAGCTATTGAAGTCTGAAACCACAGGTTTAAGAGGGGAGGCA
CTTTTTTAACTGAAAGCTGATTGCTGGAGGCTTTCGGTATCCGTTCTTTCCCACCGGGCAGTGTTTGGTATAAAAAGTACCGTGCCTCTTTAAA
AACCCCAGACTTTGTAGGTCTGGGGAAGAAGACCTTTGCATTACAAAAACAAGTTGAGTCATCTTTAACCTCTGTCTGCCCCGCCTTCTGCAGA
GTGTCAGCACCTTATTAAATGGTGCCTGTCCCTGAGACCGTCAGATCGGCCCTCCTTTGAAGAAATCCGGAACCATCCGTGGATGCAGGGTGAC
CTCCTGCCCCAGGCAGCTTCTGAGATCCATCTGCACAGTCTGTCACCGGGATCCAGCAAGTAGCAGCCTTTCTGCTGCTGTCTGTCAGACCCTC
CAGGGAGGGAGAGCTTGTCTTCTGGCCTCCAACAGGACCCTGCGTGACGATGCAGGGACAGCAATGACAACTCATTCCAGACTCCAGGTCCCTG
GAGGAGCCTCCCACAAGGGAAAGAGACTACTTCACTGGTCCTGGGCCCCTCTTTGCGCGCCCCGCCCCCAGACTCAGCGTCTAGTGTTGCTGGG
CTCCCCTGAATCCATCCCTGGTTGGGGGTGGGAGTGGATCAGAGCCCTGTCGATGGAACTTTAGTCACCATGGAGACTGGGTCACCAAGATGGG
CCGGGGGGGAGAGGAAAACAGTCTGGGGGGTGGGATAAAAACTAGCACCATATTTAAGTCCCTGTCACCTCTTCCAACTCCTGAGTGCCTTCTG
TGGGGACTCCGCCTGTGCTGGGAGAAATACTTGAACTTTACCTCTTACCTGCTGCTTCTCAAACTCTGCCTGGGTTTTGCTTCCTTCCTCTCCT
CTCCTTTCCCATCCCCCACTTCCTATGAAAGGTGCCATGGGAGAGGCTACAGGGCCAACTGCTGAGCCACCTGCCCCTTTTCTGTCTCCTTTAG
TAAAATTCCCAAGCAAACTGGTCTTCCTCTTTGGTTTCGACTTAACTGTTTCAAAACCAAGACCTCACACAGAACGCACAACCAATGCAAAACA
AAAAACAAAAAAAACAAAAAAAAAACCAGACAAGCTGTAAATGTGTGTATAGTTCCGGCATGGTAGCCTGCAAAAGGACTGTAGATGATAACAA
TTTTAAAGAAGAAAAAAAAATGCCTTTAAGTTATTTTATGTGTTGGGGGTTTGGTTTGGTTTTTTGTTTTTGTTTTTTTTTTTCTTTTTTATTC
TTTTTTGCTTGTTTTTGAAAGATGGCACGTTCTAGCCCGGAAGTTTTAATGTATTTATTTATTTATTTATTTGGTTGCCTTCCTGTCCAAGCTT
CCACTGACTGTTGCCCCCGGTTTATTGTCATGTTCTGCCTTTTTTTTTTCTTTCCTTCCTCCTGACTTGGGGACCTTTTAGGGGAGGGCTGTGA
CGCTTGCTGGGGTGTTGGGGCTCCGGTGGGACAGCTGCTGTAGCCCCCTTCTCCAGGGGCCCCCTTGGCTGCTTGCTGGAGTGGGTCTAGGGTC
TGCTGACTGTACATAAGACAGTTCCGGTGTGGTGTGCCTTCCGGATCCTCTCTGGGGCTGTGCTTTGAGCAGCGTGCAGCCCGCTGGTTTTATC
TGAGTAAAGTATACTGTACAGGGGAATAAAAGAGATCTTATTTTTTTTTTTTATACTTGGCATTTTTTGAATAAAAACCTTTTGTCTTAAACCT
TTGAGTTTTCCATTCATTCATACAAAGATGTATGCTTGGTACAGAGCCAGGTAGACTGGGCACTTTCAGACTAGATCTGATTTCCTGCTGGGGC
ACAGAGAAGTACCAGGCTTTCTGAACAAACCATGCTGGATAAAACCCAGGCCTTCCGTGCCCACTCTACCAAGGAAACTCCCTGACTATGACCA
ATTCCCTTTAACAAAAGTTTAGGGATTGTCACCTTTTCAAAGCAGGTGCAACCAAATTACAGCACCAGGAACATGGGGACAAGACTTGGCTAGG
ACTAAATTTTTCTGACGTGTCATGTCAGAGCTAGCATCGAACATTTGAGTTCCATCAGACAATGTGGTTGCAGGAATTACTACAGGTAGCATTT
GATTTGGTTTTTGTTTTAAAGATTTTATGTATGTGAGTACACTGTAGCTGTCATCAGAACAGGGCATCAGGTCCCATCACAGATGGCTGTGAGC
CACCATGGGGTTGCTGGGAATTGAACTCAGGACCTCTGGAAGAAGCAGTCAGTGCTCTTAACCATTGAGCCATCTCTCCAGCACCCTGGTCTTG
GTACCTCATAGCAGGGTTCTCTTGAGCTCTAGCTGTACTAGGGCTGTAGATGAGGCTGGCTTCAGCTCAGACCTACCGCCCGGCTTGGTGAGTG
ACCAGAAGCCTAGTGTTTTTGACACAAGGTTTGAAGTCTGTGTAGCCTTGGAACTTGATATGTATAGACCAGGCTGGCCTGAAACTCCTGGATC
TGCCTGCCTTTGCCTCCTAAGTGCTGGGATTAAGGGCATGTATAGTTCCACTGGAAAAGATTTGGGAGCCACGAGTCCTAGCATCTTCCTAGCA
GAGTGGTGTAATCCTATAATCCCAGCACTTGGGAACTAAGAGGATGAGAGTCAGGAGTTCAAGGTCAGTCTCAGTCTGGGATACTCGTGACTGG
TCTAATCAGCTGGAAAGATGCACTCTGGTAGACAGGACCAAAGGAATTC
MOUSE SEQUENCE—CODING
ATGCTCCTGTCCAAGATCAACTCCCTGGCCCACCTGCGCGCCCGCCCCTGCAACGACCTGCACGCCACCAAGCTGGCGCCGGGCAAAGAGAAGG
AGCCCCTGGAGTCGCAGTACCAGGTGGGCCCGCTGTTGGGCAGCGGTGGCTTCGGCTCGGTCTACTCTGGCATCCGCGTCGCCGACAACTTGCC
GGTGGCCATTAAGCACGTGGAGAAGGACCGGATTTCCGATTGGGGAGAACTGCCCAATGGCACCCGAGTGCCCATGGAAGTGGTCCTGTTGAAG
AAGGTGAGCTCGGACTTCTCGGGCGTCATTAGACTTCTGGACTGGTTCGAGAGGCCCGATAGTTTCGTGCTGATCCTGGAGAGGCCCGAACCGG
TGCAAGACCTCTTCGACTTTATCACCGAACGAGGAGCCCTACAGGAGGACCTGGCCCGAGGATTCTTCTGGCAGGTGCTGGAGGCCGTGCGGCA
TTGCCACAACTGCGGGGTTCTCCACCGCGACATCAAGGACGAGAACATCTTAATCGACCTGAGCCGCGGCGAAATCAAACTCATCGACTTCGGG
TCGGGGGCGCTGCTCAAGGACACAGTCTACACGGACTTTGATGGGACCCGAGTGTACAGTCCTCCAGAGTGGATTCGCTACCATCGCTACCACG
GCAGGTCGGCAGCTGTCTGGTCCCTTGGGATCCTGCTCTATGACATGGTCTGCGGAGATATTCCGTTTGAGCACGATGAAGAGATCATCAAGGG
CCAAGTGTTCTTCAGGCAAACTGTCTCTTCAGAGTGTCAGCACCTTATTAAATGGTGCCTGTCCCTGAGACCGTCAGATCGGCCCTCCTTTGAA
GAAATCCGGAACCATCCGTGGATGCAGGGTGACCTCCTGCCCCAGGCAGCTTCTGAGATCCATCTGCACAGTCTGTCACCGGGATCCAGCAAGT
AG
HUMAN SEQUENCE—GENOMIC
GGCTAATTTTGTATTTTTAGTAGAGATGGGGTTTCTCCACATTGGTCAGGCTGGTCTCGATCTCCCGACCTCAGATGATCTGCCCGCCTCGGCC
TCCCAAAGTGCTGGGATTACAGGCATGAGCCACCGTGCCTTGCCAACCCGTCTCTTTAAAAAAAATTTTTTTTACGGCCAGGCACGGTGGCTCA
CGCCTGTAATCCCAGCACTTTGGAAGGCCGAAGCAAGTGGATCACTTGAGGTCAGGCGTTCGAGGCCAGCCTGGCCAACGTGGTGAAACCCTGT
CTCTACTAAAAATACAAAAATTAGCCAGGTGTGGTGGTATGCGCCTGTAATCTCAGCTACTCGGGAGGCTGAGGCAGAAGAATTGCTTGAACCA
GGGAGGCGGGGGTTGCAGTGAGCCAAGATCATGCCATTGCACTCCAGCCTGGGCGACAGAGAAGACTCCATCTGAAAAAAAAAATTTTTTTTTT
TTACAATATGATCTCTGCACTTGAAGAGCTGATGGTCTCATTGGGGAAACATGAGTCCTTTCAAAGTGGGTATTAAATTAATGCTTGCTGCTCC
TTAAAATGATGCTATAATGACAGTAACTACTTAGATAACAAAAGAATATGATTGTATCAGATTAGTATTACAAGTAACTGCTGTGAGTTTAAAA
ACTATTGGCTGGAGTGGTCAGGGAAAGCTGTAAGAAGTCCAAATGGCCTTCAGTTGGTCTGAAAGGACAGACAGAATTTGGGGATGTGTGTGCA
ATTTGGGTTAAAGAATGTAATCTGTGTTGTCTTTAAGCCCTGCATCAGAGGACCAGCTATTTGCCAACATAACTCCCATTTACAAGAGTAACTT
CAGATAAAACCCTGATGACTGCCAAGTCTGACTTTCTCATCTAGTGAGTGGGTGTCTCTCTAATAAAGTAGCATCACTGAACACAGAAGCAAGC
TTAACCTATCCAGGGAAAAGTGACACCATTTCTATAAAGGGAACAGCACAGCTCCCTAGTTCTCCAGAGTGAGCAAGATTGTATTTAGACATGC
AAAGAATCTCTGACAAAGTAATACATTGAAGGCCATAAAAAACAGCTCTGTATCTGTAGTATAGTTGTTCTAGTTCTGGCTGTGGTGAGCTGTC
ATGTTTGTTTTAGCAGCATGCCACCTAAAAAGAGAAGTAAAACAGAGTTGGAAAAGAGGAAGTATATGTTGAAAATGTAAGTTTCTCCAAATTA
TTGAGTAGATTTCGTCCAATAATGTTACTACATGTCTTTTTTATCATTTGGAACTTGGAACACTCTGTGTATTAACCACGATATAAGCAAATAT
TCCTAAACTATAAGCTTCAAAGGATTATTTGAATAGAGAATCTTCCTAGTTTCTTCTTCAGTAGACTGAAATAGCCAGGCACAGTGGCTGACGC
CTGTAATCCCAGCACTTTTGGAGGCCGAGGTGGGCAGATCACGAGGTCAGGAGTTCGAGACCAGCCTGGCCAACATAGTGAAACCCTGTCTCTA
CTAAAATCACAAAAAGTAGCTGGGCATGGTGGCGCGTGCCTGTAGTCCCAGCAACTGGGGAGGCTGAGGCAGGAGAATCGCTTGAACCTGAGAG
GTGGAGGTTGTGGTGAGCCGTGATCGTGCCACTGCACTCCAGCCTGGGCAACAGAGGGAGACTCTGTCTCAAAAAAAAAAAAAAAAGACTGAAA
TAAAATATCTGTCATCTTTTGCAACATGACATCAGGAGCTCCTACTAATTCTCTCTGGCTTCTTGCTTTTGATTTAAAAACTTTTGAGGCCGGG
AGCGGTGGCTCACGCCTGTAATCCCAGCATTTTGGGAGGCTGAGGTGGGTGGATCACAAAGTGAGGAGTTCAAGACCAGCCTGGCCAAGATGGT
GAAACCCTGTCTCTACCAAAAATACAACAATTAGCTGGGTGTGGTGGTGCACACCTGTAATCCCAGCTACTCGGGAGGCTGAAGCAGAAGAATC
GCTTGAACCCATGAGGCAGATGTTGCAGTGAGCCGAGATCGCGCCACTGCACTCCAGCCTGGGTGACAGAGCGAGACTCCGTCTCAAAAAAAAA
AAAAAAAAAAAAGAAACTTTTATTCATTCATTTCATGCGTATTGGGGAATACAAAAAAACAACACAAAGCATCTTTTGCTTCACTTCCTGACTC
TACTCACCGTGTTCTCTCTCTCCTTCCTACTTACTCGTATTTTACTCATTAAGACCTGAGTTAAGATCTACATCCTCCATGGAGCCTCCCCGGA
CCTCTTCAGCCTCCTCTGAACCTTGACTTTGTGGATGATGCTATCAGAACTTAATCACAGAAGACCTTGTTAAATCTCTTTTTTTGTTTGTATG
TTAGAGACAGGGTCTGACTCTGTCGCCCTGGTTGGAGTGTAGTGGCACGATCAAGGCTCACAGCAACCTCTGCTACCTGGGCTCAAGCAGATCC
TCCTCCCGAGCTCAGCCTCCCGAGCAGCTGGGACTACAGGTATATGCCATCACATCCAGCTAATTTTTGTATTTTTTGTAGAGACGGGGTTTCA
CCATATTGGCCCACCTGGTCTTTAACTCCTGGGCTCAAGTGATCCACCAGCCTCAGCCTCCCAAAGTGCTGGGATTACAGTCATGAGCCACTGT
GCCCGGCCGTTAAATCTCTTAAACTCATTTTGTGTTCCTTGGAAAGGCAGAATGGCATGGTGGGTAGGAGATTGCTCTGCCACTTAACAGTGTA
GCCTTGGGCAAATTACTTATCTCTGTTCCGTTTATTTATCTGTAAACCAATGAAGATAGTAAAAATGCCCACCTCATAGGATTTATATGGGGAT
ATAAATAGAACAATGCCTAGCACATCATTGATGCTCAATATTGGCTTTTAGTACCCTATCTCTTTTGCCCCTCCCGCCAATGATTTTCATTTCT
TAGCCTTTATGTTCAATTAAAGGATGATGGTTTCTGCCTTCAATATATTTATAGTCTAGTTGTAGAGATGAGACAAATACAGAAAAATAAGACT
ATATACTATTAGATGACAAATTAGATACACAATAAAGAAATGTTTTCATCACTTTCTTTGTAGTGCTTACAAGGATGTTCCATGAAATCTTTTT
TGCACTTTTTTTATTTTACCTTGATGAACTTTGTGGCTTTTCCAAAGTCTTACATCAGCAATTTTATTTTACTTTTAAAAGATGGTAGACAATC
CCCTCACCAGCTCCTAGATAGAAGACATTCTCTCTCTCTCCCTCACACACACACACACACACACACACACACACACACGCACAGACACGATGGC
CCTTTTCTTTGTTAGTTAGACATGCAGCGTTTTTGTTCATGTGTCTGCCTGGTTGTTCTGATCTGAGGGATACATTTTCTTAGTCTTCTAATAA
GGTATTTTATAACAGTCTCCAGGCTTCCTGTAGCCTTTATATGTTCTAAGCTACTCCTTTTTATATTCATCTATCCAGCAATACTCAGGAACTA
TAAAATCTTTGTGTATAATGCCTTCAGTAAATATTATTTTTACTCAAATTGTCAGTATCTAATGTGCAAAATTCCAAAAATAAATCTAGATTAC
TTTTATTTCCTCTGAAAAACAAAACAAATGAATTGGTAATAATTTAAATATACTTTATTGTAAAATAGCCCTTTCTTTTCCCAACCTTGCTGGC
TAAAATAGATTTATAAAACTGAAATAGGCCAGGCGCAGTGGCTCATGCCTGTGATCCCAGAACTTTGGGAGGCCGAGGCGAGCGGATCACGAGG
TCAAGAGATCGAGACCATCCTGGCCAACATGGTGAAACCCCGTCTCTACTAAAAATACAAAAATTAGCTGGGCGTGGTGGCGCATGCCTGCAGT
CCCAGCTACTCCGGAGGCTGAGGCAGGAAAATCCCTTGAGCCCAGGGGCCAGAGGTTGCAGTGAGCTGAGATCGCACAACCGCACTCCAGCCTG
GCGATAGAGACTCCGTCTTAAAAAAAAAATGAAATAAATATCATATCCTTATCAAGGACAAGAACTAGCTAGGCATGGTGCCTCACACCTGTAA
TCCCAGCACTTTGGGAGGCCAAGGCGGATGGATCACGAGGTCAGGAGCTCGACACCATCCTGGCTAACACGGTGAAACCCTGTCTCTACTAAAA
ATACAAAAAATTGGCTGGGCTTGGTGACACACGCCTATAGTCCCAGCTACTTGGGAGGCTGAGGCAGGAGAATTGCTTGAACCCGGGAGGTGGA
CGTTGCAGTGAGCCGAGATCGCACCACTACACTCCAGCTTGGGCGATAGGGTGAGACTCTGTCTAAAAAAAAAAAAAAAAAAAAAAAAAAAAAG
GCCAAGAACTAGTTCTAGTGACTACATAGATTCCTTGATCATTCTGAAATTGGGACATGGTGGATTTCCTAAGTGTCTCCCACTATCACAATGA
TTTCAATTTAAAAAGGTTTTCAGCTGGGTTTAGTGGCTTATGCCTGTAATACCAGCATTTTCGGCCGGGCGCGGCGGTTCACGCCTGTAATCTC
TGTAATCTCAGCACTTTGGAGGCCTAGGCGGGTGAATCACCTGAGGTCAGGAGTTTGAGGGCAGCCTGACCAACATGGTGAAAACCCGTCTATA
CTAAAAATACAAAAATTAGCCGGGTGTGGCGGCAGGCTCCAGTAATCCCAGCTACTCTGGAGGCTAAGGCAGGGAGAATGGCTTGCATCCGGGA
GGCAGAGGTTGCAGTGAGCCAAGATTGCGCCACTGCACTCCAGCCTGGTAGACAGAGCAAGACTCCATCTCAGACAACAACAACAACAAAAGAA
ACGCATTTTGGGAGGCCAAGACTGATGGATCACTTGAACCCAGGAGTTTGGGACCAGCCTTGGCAAAATGGTAAACCTCATCTCTACAAAAAAT
GTGAAAATTATCGGCCGGGTGCGGTGGCTCAGGCCTGTAATCCCAGCACTTTGGGAGGCCGAGGCGGGCGGATCACGAGGTCAGGAGATCGAGA
CCATCCTGGCTAACATGGTGAAACCCCGTCTCTACTAAAAATACAAAAAATTAGCTGGGCGTGGTGGTGGGCGCCTGTAGTCCCAGCTACTCGG
GAGGCCGAGGCAGGAGAATGGCGTGAACCCGGGAGGCGGAGCTTGCAGTGAGCCGAGATCACTCCACTGCACTCCAGCCTCGGTAGCAGAGCGA
AACCCCGTCACACACACAAAAAAAGAACAAGTGAAAATTAGCCCGGCGTGGTGGCTCACCCCTGTAATTCCAGCTAGGTGGGAGGCTCAGGCAG
CAGGATCGCTTGGGCGCAGGTCTAGGCTGCAGTGCGCCGTGATCCATTGTGCCACTGCACTCCGGACCCTCGGTGACCAGAGTGAGACTTTGTC
TCAAAAATAAATAAATAAAAATTAAAACAAAAAAAGAAATATTCAGTTTTTGTTTTGTAAAGACAGGGTTTCCCTATGTTGCCCAGGCTGCTCT
CCTGAGCTCAAGCAATCCGCCTGCCTCGGCCTCCCAAAGTGCTGGGATTACAGGCGTGAGCCACCATGCCTGGTCTTTTTAAATTTAGAATGTT
TCCTGTCTGGCCAGGCGCGGTGGCTCATGCCTGTAATCCCAGCACTTTGGGAGGCTGAGGCCGCCGGATCACTTGAGGTCAGGAGTTTGAGACC
AGCCTGGCCAACCTGATGAAACCCGTCTCTACTAAAAATACAAAAATTAGTCGGGTGTTGTGGCGGACGCCTGTAATCCCAGCTACTTGGGAGG
CTGAGGCAGGAGAATCGCTTGAACCCGGGAAGGGGAGGCTGCAGTGACCCGAGATCATGCCACTGCACTCCAGCCTGGGCGAGAGTGGGACTCT
GTCTCAAAAACAAATAAACTAAATAAAATAAATAAAAAACTGAGTAAATTTTTTTTAGGAGATAAATGAGTGATGCAGATTCAATACGTCTCTA
CCTGATTAATTATAAATAGCGTATAGAGTCTTAGTTATACAATCCTAGTTATATAAATGCATTGACTAGACTTACAGACACTTTAAAAAATTGT
GTACCGATATCACTTTCAAACTTTCTTCTAAACTTGTTTTTTAAAAAAATTTCGTGACTACAGAACTTGCTGAAGTCATTGTTTTTTTGTTTTT
TTTTTTTGCATAAGCATATAGTCCCTGGATGAAGTCATTCCTTTGACAATGACTTGATTAAACCCATATCAGGCAGAATATGAAAACCACCTAA
GGATTAATTTGGATATTGTTCAAATTAATATTAATAAATGCTCCTCTTCCCAGAGAACTGTTAGCATAATTGTGGCCTTTTATCCTTCCAAAAG
CACTTGTCATTTAATTATTAGGGTGATTTTTCTGATTCTTTGTTAGATTCTTTGAGATAGCACAAATAGTGCTAATTTTATTAAGACTATATTT
CGATAAATCAAAACATCAAGTTCCCTACTCTCGAGGAGTAACTTTTGACCCACAACACATCTGAATGTTTACTAATTATCTTTTAAAGCAGTTT
TAAGTAAATCAAAGCTGTTTGCCAAGCAAGGTCCATGTTTTGTAACTTCCAGCTGTGAAGCCCCCACTATACAAAGTAGATATATTAAATGTTA
ATCACTTTAGCTCTAATTAAATATTTAGTCCTTCAAAGACCTAGAAAAAGTGAAAAAGGTTGAAGTTGATACATATTTTTTTTTCTAAAAGGCC
TAAAAGATCTACTAGAAACAACTATTTTAAATCACATCTCTCCCCAAATGGTTAAAAATCGAACTATTAATCCAGTAGTTTACCTTTGGTACCT
TCTAGGTGTAAGACCGTTTGGGCTGGGCGCGGTGGCTCACGCCTGTAATCCCAGCACTCTGGGAGGCCAAGGCAGGTGGATCACAAGGTCAGGA
GATCGGGACCACCATGGCTAACACGGTGAAACCCCGTCTCTACTAAAAATATAAAAAATTAGCCAGGCGTGGTGGCAGGCGCCTGTAGTCCCAG
CTACTCGGGAGGCTGAGGCAGGAGAATGGCGTGAACCTGGGAGGCAGAGCTTGCAGTGAGCCAAGATTGCGCCACTGTACTCCAGCCTGGGAGA
CAGAGCGAGACTCCGTCTCAAAAAAAAAAAAAAAAAAAAAAAGGCCGTTTTAAAAAACATCATCATCATAACCACGATCAACATTATTGCCATC
ATCATTGCTAATATTTATTAGAAGTTTACTCTGTACTGGGTTATATGCTTTACAAACAAGAGTTTTATTTGCTCCTTACAACAACCCTATGAGG
TAGATGCTGTTATTATCTCCATTCTGTCTAAAGCTTAATAAGGTTAAGTAACTTGACCAATGCCTAACTAGTAGCTGCCAGATCCCGGAATTGA
AACCAGGCTAACTTAGTTTAAAGCCCTTGCTCCCAACTTTTATGTTCCCGAAGCCCACTTAATTCATTTAAATTTGGTATAAATTGACTTTAAT
TATGGCCTTTTTGTTAGAATCCATTATAGTCTTCTCTTTTATCAACAAATGTTGGATAAAGTATGCATTCAGGGAGGCTGGGCATGGTGGCTCA
CACCTGTAATCCAAGGAGGCTGGGCATGGTGGTTCACACCTGTAATCCCAGCACTTTGGGAGGCCGAGGCAGGCGGATCATAAGGTCAGGAGTT
CGAGACCAGCCTGGCCAACATGGTGAAACTCCGTCTCTACTAAAAATACAAAAATTAGCTGGGCATGGTGGCGGGCGCTTGTAATCCCAGCTAC
TTGGGAGGCTGAGGCAGGAGAACCAATTGTTTGAACCCGGGAGATGGAGGTTGCAGTGAGCCGAGATAGTGCCATTGCACTCTAGCCTGGGTAA
CAGGGCGAGATTCCGTCTCAAAAAAAAAAAAAAAAAAGGGCATTCAAGGAATAGGAAGACAAAGTATAATGGAAAAGTAAATGAACAAAAAGGA
TTTAAAAAAACAAATCCCAACTGTCAGATTAGTGGACAGTGAACCAATGCTCTGAGTTTGTTCATCAATCTGTGGTAGAACTACAATTGTTTCC
TAAAGAGTGGAAAGGCTTTGAATAAAAGTGGAGAGGTAGAGAGGGCTCTCCCAGGACAGGTTGATTGCTTCCGCAAAGGCAAAGGGTCTGTAAA
GTGCTAGGCGAGTGGGAACAAGTGGGAATAGTTCTGTTTGACTACATTTGAGGGTTTGAGAAAGGAAAGGGAGGGTAGAAGGGATGGAAGTGGG
AATCACATATGTTTAATGATGTCTTGTTCGGTGCCAAGGACAGTTTTAGACCCTGATAAGCGGAAAGCTTTGAATACTCAGGAGGTGAATTTGG
GTCATTTCATCTCCTCTCTTAGCTGCTGTATGACCCTGTGCCATCCTATTTATGTTTCATTTTCCTATCTGCAAAAAGGGAGTAATTCTTGTTC
CCTTGGGTTTTCTCCTAGTCATAAAAAATGAATGAGTTCGGCCACAGTACACAAACAAAAGGAAAATATGTAATATTTTGTAGGATAAATTCAT
AAAAGTTGTGGAGGATCTGGGCACTTTTATAAAGCAAGCACTGTAGAAAACTTTCAAAGGAAACATTTAAATTCACTAATGACAGCTATAGGGA
TCCTGATTTTTCCTTTAAATGGCAGGCACTTCAAAAAATTAAAATAGAATGTAGTTAGCTTTCAATTACTTAGGCCACTGACAATTCAATTTAT
GGATTATATAGTATTTTAATTTACTCATTTCACACGTCGTTATCAACTCTACATGAAGTTTAAAAGGACAAAGGATGCAAGTGGAATTGGTCAG
TGTTCCGGGTTATTTCCAAGGGAGGCAGAGGGTGGGGGGTTTCCTTTGAGACAAGACTTGGGGTTGGCCAATAATTGCTGGTATTGCCTGCCTG
GTAATAACAGGCTGATGAAAAGGTGACTACAACGTGAAAACTGGTTAAATCAAGCGCACCCTCCCACCCTCGTTTTAGATGAGGAATTTTCCGC
CCTCACAGAAGGGGCTGAGGCAGCATCTGGCATCACAACACTAACATTTGCTTCGTGATTTCCTCTTTACCCGGCCCTTTGACACACATCCCTT
CCCAGAAATCAGGATTCGCTGGTGCTTTTGCATTTCTAAAATGGGAATCCCGTGGCTGAGCTTTTAGCCGGCCGGAACGACTGAGGGCTGCATC
CCTTTCCCGCAGGAGCGGGGCTCCCGCCTCCAGTTCTGTTCCAGGCCTGACTCCTCCACTCCCTCCGTGACTCATGTCCTGCGGATCCTTCGCC
CCCGACGCGCCCCCCAACACACAAACCCCCAGAATCCGCCCCCAGCCTACAGCGCGACGTCAGCCCGCCCCAGCCGACTTGGAGGTCTCGGGTC
TGAGTCACACAGAAAGACCACCCTCGTCGGCATCCCCACACACAGTCCGACACCCGGCGCGCCGGCCTCCCCGCCTGACACACTAACGCCCGTC
GTCTCCGCGCAACTTGTTATGCTCCGGCTCGAGCCCTTGACCCAAAAACCTCAGCGAAACGGAGAGCCGCAGAGCCGGCCTCGGGCGGCCTTTG
ATGGCTTTGTTATTGTTTGGGTTTGAATCGATACGCCCCTCCCCATCCTTCCTCCCTCGCGGCCCTACACCCAGCTCCCGCCTCCCCTCACGCC
CCGCGCCCCTCCCCCTCCATTTTGGCGCCTTTTCCTTCCCGCCACGTCGTGGCGGCGTAGAGACCATTCTGACCGCGAGAGCTGGGCGGGGCGG
GGGCGGGGCGCGCCGAGTTATGCAGATCAATCGGCCTCTGGTTGGCTGGAGTAGCGCTGGCAGGGGCGGGGCCGGGGCGCGGCCACAGAGCGCG
CGGGGCGGGGGCCGAGGGGAGTCGCCCAGTCCCGCCGCTTCCCCACCCCCTCTCCTCCCTCGGCCGGCCCGGCAGCCCTGCTCCCCGCCTTGGC
CTCCCGGAGAGGCCCCGCCCCGTCCCCGCCCGCCGCGCCCTCCCCGCGCGCCCTCCCCGCCGGCGCGCTCCTCCCCTTTACTCCTGGCTGCGGG
GCGAGCCGGGCGTCTGCTGCAGCGGCCGCGGTGGCTGAGGAGGCCCGAGAGGAGTCGGTGGCAGCGGCGGCGGCGGGACCGGCAGCAGCAGCAG
CAGCAGCAGCAGCAACCACTAGCCTCCTGCCCCGCGGCGCTGCCGCACGAGCCCCACGAGCCGCTCACCCCGCCGTTCTCAGCGCTGCCCGACC
CCGCTGGCGCGCCCTCCCGCCGCCAGTCCCGGCAGCGCCCTCAGTTGTCCTCCGACTCGCCCTCGGCCTTCCGCGCCAGCCGCAGCCACAGCCG
CAACGCCACCCGCAGCCACAGCCACAGCCACAGCCCCAGGCATAGCCTTCGGCACAGCCCCGGCTCCGGCTCCTGCGGCAGCTCCTCTGGGCAC
CGTCCCTGCGCCGACATCCTGGAGGTTGGGATGCTCTTGTCCAAAATCAACTCGCTTGCCCACCTGCGCGCCGCGCCCTGCAACGACCTGCACG
CCACCAAGCTGGCGCCCGGTGAGAGCACCCCCCGCCTCCGGCCCGGGGATGCGGGGCGGCGGCGGGATCTCCTGGGTGGGGAGCTGGCGGCTCG
CGGGCCGGCACTGAGTCCCCGTGCTTCCCCCTTTCCTAGGCAAGGAGAAGGAGCCCCTGGAGTCGCAGTACCAGGTGGGCCCGCTACTGGGCAG
CGGCGGCTTCGGCTCGGTCTACTCAGGCATCCGCGTCTCCGACAACTTGCCGGTGAGTGGGCGCCCCGCGGTGGGGAGGGCGCGCCGGGCGGGG
GGCGCACGGGCGTGCTTTAGCCCGGACGAGGGAACCTGACGGAGACCCTGGGCTTCCAGGTGGCCATCAAACACGTGGAGAAGGACCGGATTTC
CGACTGGGGAGAGCTGGTGAGTGCCCTGCAGGAGCGACCCCCAGGATGAGTGGGTGGGGTGAGGGGCGCCCCCGACTCCCGCCCTAACGCGGCC
CCCTCGCCCCTGCAGCCTAATGGCACTCGAGTGCCCATGGAAGTGGTCCTGCTGAAGAAGGTGAGCTCGGGTTTCTCCGGCGTCATTAGGCTCC
TGGACTGGTTCGAGAGGCCCGACAGTTTCGTCCTGATCCTGGAGAGGCCCGAGCCGGTGCAAGATCTCTTCGACTTCATCACGGAAAGGGGAGC
CCTGCAAGAGGAGCTGGCCCGCAGCTTCTTCTGGCAGGTGCTGGAGGCCGTGCGGCACTGCCACAACTGCGGGGTGCTCCACCGCGACATCAAG
GACGAAAACATCCTTATCGACCTCAATCGCGGCGAGCTCAAGCTCATCGACTTCGGGTCGGGGGCGCTGCTCAAGGACACCGTCTACACGGACT
TCGATGGTGAGCCAGGCCCGGGAGGGAGCTGCCCAGGTGACTCGGCCCGGCCCGGCCCAGTCCGGAGGCCTCGGCCAGTCTCCCGCGCCAGCCT
TTTGTAAAGGTCATTGGGCCGCCTGGCTCGATGCTAGCCGGGGTGGGACGCAGGAGAGCCTCCCAGCGTAGTAAAGCCGGGGATTTTCAGCCAG
CTGAACCTGTAATGTTTCTGGCATGATTTTATTCTTCAAGTGGAATTCAGTTAGTTCCAGGCTTTCCCGATGAATAAGAGGTTGTGGGCAACCG
GCGGTAGCCCAGATTTTTCTAAAGTCTGACCCAGTTTCCCCGCCAGTAAAAGGATGGGGGCGGGGGAAGAGGTGGAAATTGATGCCGGTTTTGT
AATTTTTGTTTTATTTTATAAGGGAGTTAGTTTTCTGTATGGTAGTTTTAGAGCTGCAGTTCTTAACTCCTTTCTGATCTAGGGAAGGTTAAGG
AATAGGAACTATATTATTACTGGTGGCTTTTTTTTTTCTTTAGTGTTAAGGGGAGAGAGAGTCAGGAATGAATGTTGTGAAATAAGATCTGTCG
CTGGTTTGAAAATTAGTTGGGTGTCTCCGCAGAGAGGATGAAAACCTATCCTAGGGAGGGGCTTGGAGCGGGTTCTTTCAGAAAAGAAGGAATG
GAGAGCCTGAGATCAAAGCTGCGGAGGGTGGGTCATCATCTGAGCGGCTTAACCTAACAAACGACAGCCTTTCAAAACTTGTGACTCGGGCTTG
TGGTTTATGTTTATTTGCCCTTGGAGGACGCTGGGTTGGGCTGCATTTTTTGTATTTAACAGTTAATGGCTGGCCGGGTCCTCCCATGTTTTTT
CTTTCAAGTCTTTGCTGCCCCCTGGTGAGCACCAGCGGCATGGCCCCTCCTTTTTCTTTTACTACCCAAAGTTTGTAAACAGGAATCACGTGGT
CTGAAACCAACCCTGCAGCTCTGTCTACCTTTCCAGTCTAGGGAGGAAAGGGTGTGGGTGTCTGCTCCTGCTTGCATCGGGTGGGGAGGAAGGC
CTCCCAAAGGGCACCCTGACTTAGGATGTTGTGCAAGCATCCTTGCTTGGATCCTGTCGGCCATGAGAATCTCACCCGGGCTCCTGGGCAGTGG
TGAATGCAATTTAAGGATAGTCTATGAGATACCTTTCTTGGTTGTGCAGACATGCATCCCTTCATCCTTCGCAGGCGGTCCTGCCTCACAGGGC
CTCAAGTTTTGGGTCTGCGGCCAGCTGTGTTTGTTTCTTGGAGCAGTTCATAAAGAATTTCAGTTTATGGTTTGGGCTAGCAGAGAGGTGGGTA
ATGCTTTGGGTTGGAGAGATGCCGTAAGGTGCGCCTCCACTCTCCTTAGCCCAGAGGGAAAAATGGAGTTCACCTAGCTCCTGAGAGAAGGGAT
TTTTTTTTTTTTTAAAAGAAAGAGTTATATATACCACCCAGCTTCTTTGTGCTTGTTTTTGCTAAAAGTGTGTTTTCTCTTCTATTCCCTTGGC
TCACAGGGACCCGAGTGTATAGCCCTCCAGAGTGGATCCGCTACCATCGCTACCATGGCAGGTCGGCGGCAGTCTGGTCCCTGGGGATCCTGCT
GTATGATATGGTGTGTGGAGATATTCCTTTCGAGCATGACGAAGAGATCATCAGGGGCCAGGTTTTCTTCAGGCAGAGGGTCTCTTCAGGTAAC
TGATGGAAACCCCTGGCCATGGGGTTATTGGTCTTAATGGGGCTATTAGTCTTCATGGGACAGTCTTTGAAATTCTGGAGAGCTTCACTCTCCA
GTAGATTCTGTCACCCTTGGCTTAGAATTGTAGGTGAGTGATTTACACTTGAGCTGGCCTCATAAATCACATGGTTTGCACTTGAGCTTTCCTT
GGGAGGTCAGAGGAAGGCATGTGTGAGCATATTAAGAAGAAAAGACAATCTGGCTTCTCCAAAAACTTTTTTAAAGGTACCAACAGAAACCTGA
TAATTCCTGGCTGTTTTGCCAGGGAGTAAAAAGTTAAAAGCTCTTTTAGCATCTTCTTTAAGGCAGCAGCTCCAAATATTTTGGTACCAGTGAC
CTCACTGTGGGTGGTGTTCGTGTTTGTAAGTTGGTAGGTGAATTGAATCATTTCATCATGCTCAGTGGTGTCTCATCAAAATCTCTTGTCATCA
TCCTTCCTATTTCTGGTGAGTGGGTGTTGTGGGAAAGGCCCCCACTATTGAAGTTTGAAAACCACAGGTTTAAGAGGGGAGTCAGTTTTTAGCT
GAAAGCAGACTGGAGGACCCAGATATTAGTCAATACCTTCCTATTGAAGGGTACCCAGCACAGTGTTCTAGAAAATGCTTGGCCTCCCTGGGAC
CCCAGACTTGTGGGCCTCTGAGAAGCAAATGGGGAAGACCTTTGCAGTGTAAAAACAAGTTGAGTCATTCATAACCTCGTCTATCCTCCTTTCT
GCAGAATGTCAGCATCTCATTAGATGGTGCTTGGCCCTGAGACCATCAGATAGGCCAACCTTCGAAGAAATCCAGAACCATCCATGGATGCAAG
ATGTTCTCCTGCCCCAGGAAACTGCTGAGATCCACCTCCACAGCCTGTCGCCGGGGCCCAGCAAATAGCAGCCTTTCTGGCAGGTCCTCCCCTC
TCTTGTCAGATGCCCGAGGGAGGGGAAGCTTCTGTCTCCAGCTTCCCGAGTACCAGTGACACGTCTCGCCAAGCAGGACAGTGCTTGATACAGG
AACAACATTTACAACTCATTCCAGATCCCAGGCCCCTGGAGGCTGCCTCCCAACAGTGGGGAAGAGTGACTCTCCAGGGGTCCTAGGCCTCAAC
TCCTCCCATAGATACTCTCTTCTTCTCATAGGTGTCCAGCATTGCTGGACTCTGAAATATCCCGGGGGTGGGGGGTGGGGGTGGGTCAGAACCC
TGCCATGGAACTGTTTCCTTCATCATGAGTTCTGCTGAATGCCGCGATGGGTCAGGTAGGGGGGAAACAGGTTGGGATGGGATAGGACTAGCAC
CATTTTAAGTCCCTGTCACCTCTTCCGACTCTTTCTGAGTGCCTTCTGTGGGGACTCCGGCTGTGCTGGGAGAAATACTTGAACTTGCCTCTTT
TACCTGCTGCTTCTCCAAAAATCTGCCTGGGTTTTGTTCCCTATTTTTCTCTCCTGTCCTCCCTCACCCCCTCCTTCATATGAAAGGTGCCATG
GAAGAGGCTACAGGGCCAAACGCTGAGCCACCTGCCCTTTTTTCTGCCTCCTTTAGTAAAACTCCGAGTGAACTGGTCTTCCTTTTTGGTTTTT
ACTTAACTGTTTCAAAGCCAAGACCTCACACACACAAAAAATGCACAAACAATGCAATCAACAGAAAAGCTGTAAATGTGTGTACAGTTGGCAT
GGTAGTATACAAAAAGATTGTAGTGGATCTAATTTTTAAGAAATTTTGCCTTTAAGTTATTTTACCTGTTTTTGTTTCTTGTTTTGAAAGATGC
GCATTCTAACCTGGAGGTCAATGTTATGTATTTATTTATTTATTTATTTGGTTCCCTTCCTATTCCAAGCTTCCATAGCTGCTGCCCTAGTTTT
CTTTCCTCCTTTCCTCCTCTGACTTGGGGACCTTTTGGGGGAGGGCTGCGACGCTTGCTCTGTTTGTGGGGTGACGGGACTCAGGCGGGACAGT
GCTGCAGCTCCCTGGCTTCTGTGGGGCCCCTCACCTACTTACCCAGGTGGGTCCCGGCTCTGTGGGTGATGGGGAGGGGCATTGCTGACTGTGT
ATATAGGATAATTATGAAAAGCAGTTCTGGATGGTGTGCCTTCCAGATCCTCTCTGGGGCTGTGTTTTGAGCAGCAGGTAGCCTGCTGGTTTTA
TCTGAGTGAAATACTGTACAGGGGAATAAAAGAGATCTTATTTTTTTTTTTATACTTGGCGTTTTTTGAATAAAAACCTTTTGTCTTAACTCGT
GGCTTCTAATCGTCTGTGCGGAGGCATTGCTAACCTGCATTTATTGAGCATTTGGTAAGTGCCAAAGAATTGTAGGAGAAAGGAATTCTCAGCC
TAAATCTGGCTTCCTTCAACATTGGGATTTCTAGGATAGTCATCTTCCTGAAACGAATGTATGGTCAAGGGGTAGGACCAGTATTGTCAAAATT
TGGGCAATATCTGGCATGGGAGGCCTAATAAAATAACCTTTTCCAGATTACAATTTTAAAGCTATGCAAATAGGATAGATGTGACAATTTTCAA
GTGTTCAATAGCCATTTATGGCTAGTGGCCACCTCATATAAAACATCATTGCAGAAAGTTCTATTGGATGGGGCTGATCTAGAGATAACAAATA
CATACCTATTACCAAACTAACCCTTGAAGCTTTTATCAGTTGTCTGCCACTGATAGTAAAACATTTTGAGCATGTCCCCTTGGTACACGTGAAG
TTTATTGAAAAACTATTGCCTCTATTACTCTCGGTTCCTGATTCAAGTTTCCTTCACCTTTAGTGTGAGGTTTAGTTTCTTTTCGAGACTGAGT
CTCTGTCAACCAGGCTGGACCAGCTGGCGTGCCACTGCCAGTGGCACGATCTCGGCTCACTGCAACTTTCACTTCCCGGGTTCAAGCAGTTCTA
CCTCAGCCTCCTGAGTAGCTGGGATTACAGGCGCCCGCTGCCACGCCCAGCTAATTTTTGTATTTTAGTAGAGACAGGGTTTTACCATGTTGGC
CAGGCTGGTCTCGAGCTCCTTGACCTTATGATCCGCCTGCTTCAGCCACCCAAAGTGCTAGGATTACAGGTGTGAGCCATCGTGCTCCGCAGCC
TTTTTTTTTTTTTTTTTGAGATGGAGTGTCGCTCTGTTGCCCAGGCTGGGGTGCAGTGGTGCAATCTAGGCTCATCGCAAGCTCCGCCTCCCGG
GTTTTAAGTGATTATCCTGTCTCAGCCTCCCGAGAAGTTGGGATTACAGGCATGTGCCACCAGGCCAGCTAATTTTGTATTAGTAGATACGGGG
TTTCGCCATGTTGGCCAGGCTGGTTTGGAACTCCTGACTTCAAGTGATCTGCCTGCCTTGGCCTCCCAAAGTGCTGGGATTACAGGCGTGGGCC
ACCGCGCCTGGCCTAGTCTTTAACAATAGTATAATTTATTATGCAGCATAGAAAAAATAATAGAACGGTGTGGTGACACTAAAGTCGTGACCAG
ATAAGACCCAGTCTACAAAAAATAAGTGAAAATTAGCTGAGCGTGGTGGCATACACCTGTAGTCCTACCTATGCAGGAGGCTGAGGCAGGAGGA
TCGCTTGAACCCAGGAGCTCCAAGGTTGCAGTGAGTTATGATCACGCTATTGCACTCCAGCCTGGGCAACAGGTAACCCCACTTAAAACACACA
CACACACACACACACACACACACAATAGTGGATGGGTAGAAATGTGAGTTTGACTTAAGGTTTCCCCCTCTGATGTGTTTGACAAAGGGCTAAT
ATTGGGAGTAGGTGAAATGTTAGCTCTGCCATTTTCTTACTGCTTATTCTTGAATAATTAGTATTTTTAGTCCATGGTTTCTTTGCAGGGATCT
GTTTAATAGGCCCTTTGTCCATTTTGTGTGAGTTTAGTTAAACTGGATGGCAAGACCCATGAATCCACTTTGTACGTGTGGCCTGAATACATCC
TGATATTTTGAAAGCACACTGGAGTGCCATTGTCAATTCATATATTAAGTATTGGTAAATGTTGGTTTCTTGTTTCTTTATAAATATCCACAGT
TATAATACTCCCTCCCTGGGGGTCATCATGCTCCCAACCCTCAATGCATCCACACACCCTTTGGACACTGGCCCATATGCACCCTCGAGGTTGC
TGGAGGGTGCAGACTATAATACCCCTCACTCCTCATTGGGTTCTGGTGTATGTCTACCCTTAAGCTTAGACTAGATCCCATACCTCAGCTCAGC
CCCCATGTGGCTATAAAGAGGAAGGAAGGATGTCTTGGTTCTGGGGGGCTGATTTTCTGAGGTTCCCCTTTTCTCTAATCTCTCCCATCCCCCA
CACAACATACACACACTACCAACATCTTGGAGCCTGAGGGTTGCATGCTTCGTCCTTTCCAGTAAAGCTGACACTTGGATAATTAAAAGTTGCC
GCAAACTATTAACGAAATCAGGCAAAGCCCCACCCTTCTCTTTCTCGAAGTGAGTTTCTGGCAGTTTCCCCATCTAATGAGACTGCTGCGGGGG
AATAGAAGAAAGTATCTCTGAATAAAGACAGATCGTGCTCCTGTTGGATGCAAGAGATTAAGAGTTTTAGGGGGAGGGTGAAAGGTCATATTTA
ATCTCTTCTCAGGGGCAGTTTGACATCTAGCGTGGAAAGGGAATCAGATAACCAATAAGTCTGCGGGGGATCAATGTCATGGCCAGAGGAATAT
AGAGAGCAATGGTTAAAAGCATTGACTTGGGAATCAGCCTTGAGCTTACTTATCTAGAAACCTTGGGCAAGTTATTACACCCTCTAAAACTAAT
CTCTAAACAGAGATACTCCTGGATTGGATTATTAGGAGACTGGGTTAAATAACAGATATACTTACATACAGAAAAACTACTATCTCCCGCTTAC
GGATGCATACACATGTAGCAAAAGTACAAAGAAGTACAGGGTGGGGTAGGGTGTGGTAGTTCACACCCATAATCCCAGCACTTTGGGAAGACAA
GGAGGGATGATCACTTGAGCCCAGGAGTTTGAGACTAGCCTGGCCAACATGACAAAACCCCATCTCTACAAAAAAATAGAAGCATTAGCCGGAC
GTGGTGGTGCGTGCCGGTAGTCCCAGCTACTTGGGAGGCTGAGGTGGGAGGATCAATTGAGTCCGGGAGATGGAGGCTGCAGTGAACCAAGATG
GCACCACAACATTCTAGCTTGGGTGAAAGAGCGAGACCCTGTCTCAAAAAAACAAAACAAAACAAAACAAACAAAAAAGTACAGGGTGATAATA
AGTTTCCAGAGTCAGGGAAGTGGTTATTACCTTTGGGGAGAAGGGAGGGAGGTTGTGATTTGTGGAAAGAGACCTTCAACTATTTTGGCAATGC
TTTATTCCATAAACTAGATGGTATGAGTAAGGTGTTTGTATTATTCTTTTCAACTTTTGGGTTGTTTTAAATGTTTTATGATAAACTTTATTTT
TAAAAATTTTTCTCTGGCTGGGCGCGGTGGCTCACACCTGTAATCCCAGCACTTTGGGAGGCCGAGGCAGGCGGATCACAAGGTCAGGAGATCG
ACACCATCCTGGCTAACACAGTGAAACCCCATCTCTACTAAAAATACAAAAACTTAGCCAGGCATGGTGGCATGTGCCCATAGTCCCAGCTACC
CAGGAGGCTGAGGCAAGAGAATCGCTTGAACCCAGGAGGCGGAGGTTGCAGTGAGCCAAGATAGCTAGCCACTGCACTCCAGCCTGGGCGACAG
AGTGAGACTCCGTCTCAAAAACAAAAAAAATTTTTTTTCTTTGTTCTTTCTTTTTAAACATTTATTTATTTATTTTAGCTGGGAGTGGTGGCTC
ACGCCTGTAATCCCAGCACTTTAGGAGGCTGAGGCGGGTGGATCATCTGAGGTCAGCAGTTCAAGACCAGCCTGACCAACATGGTGAAACCCAG
TCTCTACCAAAAATACAAAATTAGCTGGGCATGGTGGTGCATGTCTATAGTCCCAGCTACTCAGGAGGCTGAGGCAGGAGAATCACTTGAATCC
AGGAGGTGGAGGCTGCAGTGAGCCACGATCACGTATAGCCTGGGCGACAGAGCGAAACTCTGTCTCAAAAAAAATATATATATTTATATATATA
ATATATAATATATATTATATATTATATTATATATTATACATAAATTATATATTATATATATAATATATAATATATAATATATATTATATATTAT
ATATTATATATTATACATAAAATATATATAATATATATTATATATTATATTATATATTATATATAAAATATATATATAATATATATAATATTAT
TTATTTATTTTGAGACAGGGTCTCACTCTGTCACCCAGACTGGAGTGCAGTGGCATAATCTCGGTTCACTGCAACCTCCACTTCCTGAGTTCAA
GCAATTCTCGTGCCTCAGCCTCCCAAGTAGCTGAGACTACAAGCGTGTGCTACCACACCTGTCTACTTTTTGTATTGTTAGTAGAGATGGGGTT
TCACCATGTTTGCCAGGCTAGTCTTGAACTCCTGATCTCAAGTAATCCACCTGCCTTGGCTTCCCAAAGTGCTGGGATTACAGGTGTGAGCCAC
TGCGCCCGGTCCATTTTTTTTTTTTCTTTTGAGAGACAGATTTTTGCTCTGTATCCCAGGCTGGAGTGCAGAGGCATGAACTTGGCTCACGGCA
CCCAGGTTCAAGCGATTCCCCTGCCTCAGCCTCCCAAGTAGCTGGGATTACAGGGACCTGCCGCCATGCTTGGCTAATTTTTGTATTTTTAGTA
GAGATGGGGTTTCATCTTCTTGGCCAGGCTGGTGTCAAACTCCTGACCTCAGGTGATCCGTCCACCTCGGCCTCCCAAAGTGCTGGGATTACAG
GCACGAGCCACTGTCCTCAGCCTTTCACTTTTTCTTTTAATTGAGACAGGGTCTCACTACGTTGCTCAGGCTAATCTTTAATTCCTGGCCTCAA
GTGATCCTCCCTCCTTGGCCTCCCAAAGTGCTGTGATTACAGGTGTGGAATATCATGCCCAGCCTATGATAAGCTTTTAAAAGTGATGAATATA
TTTGTGCGTATCATGTAGGAGGCAGCAGATGGAGTTATTACTGCCCTTGGCTGAGGGGATCTGGGAAGGCTTCAAAGAGGAGGTGGCATTTCAC
CTGGATTTTGAAGGATGGAGCCTTAGCCTGCTTCCCTGTAAAATAGGCACCAAACATGCAGATGGAGCAGAGAAATCTGGATGTGCTAAATGTT
TTTCCCCCACTAGACCAGGAATCAGCAAACTATGGCCTGCTGCCTGAACTGGTCCCTGTACAGTTTTTGTACGGCTTGCAAGCTAAGCCTAGAT
TTTACATTTTTAAGTAGTTGAAAATCAATCAAATGAAGAATAATATGTTGTGATATGTAAAAATTATATAATACTTGGCTGGGTATAGTGGCTC
ATGCTTATAATCCCAGCACTTTGGGAGGCCGAGGTGGGAGGATTGCTTGAGGCCAGGAGTTCTGGACCAGCTTGGGCAACCTAGCAAGACTTCC
ATCTCTACAAAAAATAAATAAAAATTAGTCAGGCATGATGGCCTAGCTACTTGGGAGGCTGAGACAGGAGGATCACGTGAGCCCAGGAGTTTGA
GGTTACAGTGAGCTATGATCTCACCACTGCGCTCCAGCACTCCAGCCTGGTTGACAGAGCAAGACTCTATCTCTAAAAGAAAAAGAAAAAAAAA
GAGGCCAGGTATGGTGGCTCATACCTGTAATCCCAGCACTTTGGGAGGCTTAGGTGGGCGGATCACCTGAGATCAGGAGTTTGAGACCAGCCTG
GCCAACATGGTGAGACCCTGTATCTACAAAATACAAAAATTAGCCCAGTGTGTTGGTGCCTGCCTGTAATCCCAGCTACCAGGGAGGCTGAGGC
AGGAGAATTGCTTAAACCCATAAGGTGGAGCTTGCAGTGAGCCAAGATTTCTCCATTGCACGCCAGCCTGGGCAACAGAGTGAGACTGCATCTC
ACAAAAAAAAAAAAAAAAAAATATATATATATATATATATATATACACACACACACACATTTGTACACACATATGCAACTCAATTGTCTGTGTT
CATAAATAAATCTTGTCCATTTACATATTGTCTATGGCTGCATTTGTGATACGATGGCAGAGTTGAGTAGTTAAGACACTCAGTATGGCTGGCG
AAGCTGAAAACATTTACTATCTTGTCCTTTACAGAGAAATTTGCTAATCTCTACACTAGATTATGGTCCCATTGACTCATTTTTTGTTGTTGTT
GTTTTTTTGGGGTTTTTTTTTTTGAGACAGGGTCTTACTCCGTCATCCAGGCTGGAGTGCAGTGGCTCGATCAGGGCTCACTGCAGCCTTGACT
TTCCAGGCTTAAGCAATCCTCCCACCTCAGTCTCCTGAGTAGCTGTGACTACAGGTGGGCCCCACTGTGCCCAGCTAATTTGTTTTTTTGTTTG
TTTGTCTGTTTGTCTTGAGACAGGGTCTTGCAATGTTGCCCAGGCTGGAGTGCAGTGGTGCGATCACGACACACTGCAACCTCAACCTCCTGAG
CTCAAGTAATCCTCTTGCCACTGCCTCCCAAAGTGCTGGGATTACAGGCATGAGCCATTGTGCCTGGTCTGAACTACATCTTTGATAACTCTTT
TTTTTTTTCTTTTTGAGATGGAGTCTCGCTCTGTCCCCAGGCTGGAGTGCAGTGGCATGATCTCGGCTCCACCTCCTGGTTCAAACATTCTCCT
GCCTCAGCCTCCCAAGTAGCTGGGATTACAGGCGCCTGCCACCACGCCTGGCTAATTTTTGTTTTTGTTTTGTTTTGTTTTGTTTTGAGATGGA
GTCTCACTCTGTCACCCAGGTTGGAGTGCAGTGGCACCATCTCGGCTCACTGCAAGTTCAGCCTCCCGGGTTCATGCCATTCTCCTGCCTCAGC
CTCCCAAGTAGCTGGGACTACAGGCACCCACCACCACGCCCGGCTAATTTTTTTTTTTTTTTTTTTTGGTATTTTTAGTAGAGACGGAGTTTCA
CCATGTTGTCCAGGATGGTCTCAATCTTTTGATCTTGTGATCTGCCCGCCTTGGCCTCCCAAAGTGCTGGGATTACAGGCATGAGCCACCACGC
CCGGCCTGATAACTCTTAACCTAGTGCCAGGCATGTAGTAGTTCCTTAGTGATAATAGCTAACATACAGTACCTACTATGTGCCCAGCACTATT
CTGCATGCTTTGCATGCATTAACTTATTTATCCTTAAAACAACTCTATGACACTATTGGAAAGTACAGGGGGAAACACTGTTATTTGTTTTATT
TTATTTTATTTTATTTTGTATTTTTATTTATTTATTTTTTTTTGAGACAGTCTCGCTGTATTGCCCAGGCTGGAGTGTAATGGCGCGATCTCAG
CTCACTGAAATTTCTGCCTCCTGGGTTCAAGCGATTCTCCTGCCTCAGCCTTCTGAGTGGCTGGGATTACAGGTATGTGCTATCACGCCCAGCT
AATTTTTGTATTTTTTTTTAGTAGAGACTGGGTTTTGCCATGTTGGCCAGGCTGGTCTCAAACTCCTGACCTCGGGTGATCCACCCGCCTCAGC
CTCCCAAAGTGCTAGGATTACAGGCATGAGCCACTCCGCTCTGCCTATTTTATTGTTTTAGAGATGAGGGTCTTGTCATGTTGCCCAGGCTGGC
CTCAAAATCTTGGCCTCAAGCAATACTCCTGCCTCAGCCTCCTGAGTAGCTGACTGTACAGGTACATGCCACCTAGCCCGGCTTGATGCAGTTA
TGTATCCACATTTCACAGATGGGGATAACAGATGTGCAGAGCAGTAAATTTCATGTCAAAGGCCACATATTAGGTAGTGGTCAGGTCAGAATTT
GGACCTAGGCCATCTGTGGTTCTATGGTCATCCAGACTCCGCGATCTTGGCTCACTGCAAACATTTGTGTGGATTAGCCACCTCAAGGTCCCTT
CAGGGCCCAGGATCTGGAGGTGAGAATGGTTCTTCCCCAGACTCCAGAGTTGGGTAAGCCATTCTGTTCTGTGGAAATAGTCATTATGAACTCC
TGTAAACTATCTTTCACTTGGGCCTGGCTGGGGAGGAAGAGTTGGAGGCTGAAAAGGGAGAAGAAAGGGGACCCGGGGGGGTTGGGGTGGGGCA
GGGGCACATTTCTTGTAAATGTTGCTCAGCTCCTTCAACTGCTGTGCTGGTTCTTGGAAACCTTCACCACAAAAGACCTATCAAGAACAGTGTT
AAAAAAGATATGGCCTCATCCCCCAGTTCATTTCCTGCCCAGCCTTGCACTACTAACCCCGATTGCCAAGAGCAAGGTTGCAGTGTCCCCTCAG
CAGTGCTGTGGGAGGAAGTGCAAAAATAAGCAGAACTAGGGGCAAGACCAGAGGACTGTCTTTATTTTTATTTATTATAACAGGACATCCGATG
TACAGAAGTGGATTCATCATAATCCTTGGCTCATCGTATATTCTCATCCTTGGTCCAAGAATGGTCTTTTGTTTATATATATATATATATATAT
ATATATATATATATATATATATATATATATATATATATATATATTTTTAAATCGGAGTCTTGTCACCCAGGCTGGTATGCAATGACGTGATCTC
GGCTCACTGTAACCTTCACCTCCCAGGTTCAAGTAATTCTCCTGGTTCAGCCTTCCAAGTAGCTGGGATTACAGGCGCCTGCCACCACGCCCGG
CTAATTTTTTGTATTTTTAGTAGAGACAGGTTTCGCAATGTTGGCCAGGCTGATCTCGAACTCCTGACCTCAGGTGATCCACCTGCCTCGGCCT
CCCAAAGTGCTGGGATTACAGGCGTTAGCCACCGCATCTGGCCCGTTATTATTATATTTTATCATACTGGGTAGTGAATCCACCACCCAGCTCA
AAAAACAGAATATTAACAATGACATCCATCTATCTATATGATCCTCTCTTAGTCCATTTCTTACCTCCCCCAGATACAACCCACTGTCCTAAAT
TTTGTGTTTGGCATTCTCTTACTTTATATTTTATTTATTTATTTTGAGACAGAGTCTCACTCTTGCCCAGGCTGGAGTGCAGTGGCATGATCTT
GGCTCACTGCACCCTCATCTTCCTGGGTTCAAGCGATTCTCCTGCCTCGGTCTCCGAAGTAGCTGAGATTACAGGCATGTGCCACCATGATTTT
TGTATGTTTAGTAGAGACAGGGTTTCACCATGTTGGTCAGGCTGGTCTTGAACTCCTGTCCTCAGGTGATCTGCTCGCCTCAGCCTCCCAAATT
GCTGGGATTACAGGCATAAGCCACCATGCCCGGCTTATTTATTTATTTACTTTGAGCTTGTCACCCAGGCTGGGGTAGTCGCGCAATTGAGGCT
CACTGCAGCCTCAAATTCCTGGGCTCAAGTGATCCTCCTGCCTCAGCCTCCCAAGAAGCTGAGACACAGACATGTTGCCACCACACCTTGCTAA
TTTTTTTTTTTTTGAGATGGAGTCTTGCTCTGTCGCCTCGGCTGGAGTGCAGTGGCACAATCTAGGCTCACTGCAAGCTCCACCTCCTGGGTTC
ACGCCATTCTCCTGCCTCAGCCTCCCGAGTAGCTGGGACTACAGGTGCCCACCACCACGCCTGGCTAATTTTTTTGTATTTTCTAGTACAGACA
GGGTTTCACCATGTTAGCCAGGATGGTCTCGATCTCCTGACCTCGTGATCCGCCCGCCTCAGCCTCCCAAAGTGCTGGGATTACAGGCGTGAGC
CACCGTGCCCAGCCCACCTTACTAATATTTAAATTTTTTGTAGAGATGGGTGTCTCACTATGTTGACCAAGTTGGTCTTGAATTCCTGGCCTCA
AGCAATCCTCCAAACTCAGCCTCCCAAAGTGCTGGGATTACAGGCGTGACCACTGTGCCCGGCTAACTTTTAAAATTTTTTATAGAAATAGGAT
TTTGCAATGTTGCCCAGGCTGGTCTCAAATTTCTGGGCTCAAGTGATTCACCCGCCTCGGCCTCCCAAATTGTTGGGATTACGGATGTGAGGTA
CTGCACCTGGCTAACAGCATAGCAGCATTCTTTCACTTTAAAATAACATTTTATTTTATAGGTATGTATCCCTAAATTATGGAGTATTTAGTAT
TTTTATTTTGTCTTATTTTATTTCATTTCATTTTTAGATGGAGTCTCGTTCTGTCGGCCAGGTTGGAGTGCAGTGGTGCGATCTCGGCTCACTG
CAAACTCTGCCTCCCGGGTTCAAGTGATT
HUMAN SEQUENCE—mRNA
GAGGAGGCCCGAGAGGAGTCGGTGGCAGCGGCGGCGGCGGGACCGGCAGCAGCAGCAGCAGCAGCAGCAGCAACCACTAGCCTCCTGCCCCGCG
GCGTTGCGACGAGCCCCACGAGCCGCTCACCCCGCCGTTCTCAGCGCTGCCCGACCCCGCTGGCGCGCCTCCCGCCGCAGTCCCGGCAGCGCCT
CAGTTGTCCTCCGACTCGCCCTCGGCCTTCGCGCAGCGCAGCACAGCCGCACGCACCGCAGCACAGCACAGCACAGCCCAGGCATAGCTTCGGC
ACAGCCCCGGCTCCGGCTCCTGCGGCAGCTCCTCTGGCACGTCCCTGCGCCGACATTCTGGAGGTTGGATGCTCTTGTCCAAAATCAACTCGCT
TGCCCACCTGCGCGCCGCGCCCTGCAACGACCTGCACGCCACCAAGCTGGCGCCCGGCAAGGAGAAGGAGCCCCTGGAGTCGCAGTACCAGGTG
GGCCCGCTACTGGGCAGCGGCGGCTTCGGCTCGGTCTACTCAGGCATCCGCGTCTCCGACAACTTGCCGGTGGCCATCAAACACGTGGAGAAGG
ACCGGATTTCCGACTGGGGAGAGCTGCCTAATGGCACTCGAGTGCCCATGGAAGTGGTCCTGCTGAAGAAGGTGAGCTCGGGTTTCTCCGGCGT
CATTAGGCTCCTGGACTGGTTCGAGAGGCCCGACAGTTTCGTCCTGATCCTGGAGAGGCCCGAGCCGGTGCAAGATCTCTTCGACTTCATCACG
GAAAGGGGAGCCCTGCAAGAGGAGCTGGCCCGCAGCTTCTTCTGGCAGGTGCTGGAGGCCGTGCGGCACTGCCACAACTGCGGGGTGCTACACC
GCGACATCAAGGACGAAAACATCCTTATCGACCTCAATCGCGGCGAGCTCAAGCTCATCGACTTCGGGTCGGGGGCGCTGCTCAAGGACACCGT
CTACACGGACTTCGATGGGACCCGAGTGTATAGCCCTCCAGAGTGGATCCGCTACCATCGCTACCATGGCAGGTCGGCGGCAGTCTGGTCCCTG
GGGATCCTGCTGTATGATATGGTGTGTGGAGATATTCCTTTCGAGCATGACGAAGAGATCATCAGGGGCCAGGTTTTCTTCAGGCAGAGGGTCT
CTTCAGAATGTCAGCATCTCATTAGATGGTGCTTGGCCCTGAGACCATCAGATAGGCCAACCTTCGAAGAAATCCAGAACCATCCATGGATGCA
AGATGTTCTCCTGCCCCAGGAAACTGCTGAGATCCACCTCCACAGCCTGTCGCCGGGGCCCAGCAAATAGCAGCCTTTCTGGCAGGTCCTCCCC
TCTCTTGTCAGATGCCCGAGGGAGGGGAAGCTTCTGTCTCCAGCTTCCCGAGTACCAGTGACACGTCTCGCCAAGCAGGACAGTGCTTGATACA
GGAACAACATTTACAACTCATTCCAGATCCCAGGCCCCTGGAGGCTGCCTCCCAACAGTGGGGAAGAGTGACTCTCCAGGGGTCCTAGGCCTCA
ACTCCTCCCATAGATACTCTCTTCTTCTCATAGGTGTCCAGCATTGCTGGACTCTGAAATATCCCGGGGGTGGGGGGTGGGGGTGGGCAGAACC
CTGCCAATGGAACTCTTTCTTCATCATGAGTTCTGCTGAATGCCGCGATGGGTCAGGTAGGGGGGAAACAGGTTGGGATGGGATAGGACTAGCA
CATTTTAAGTCCCTGTCACCTCTTCCGACTCTTTCTGAGTGCCTTCTGTGGGGACTCCGGCTGTGCTGGGAGAAATACTTGAACTTGCCTCTTT
TACCTGCTGCTTCTCCAAAAATCTGCCTGGGTTTTGTTCCCTATTTTTCTCTCCTGTCCTCCCTCACCCCCTCCTTCATATGAAAGGTGCCATG
GAAGAGGCTACAGGGCCAAACGCTGAGCCACCTGCCCTTTTTTCTGCCTCCTTTAGTAAAACTCCGAGTGAACTGGTCTTCCTTTTTGGTTTTT
ACTTAACTGTTTCAAAGCCAAGACCTCACACACACAAAAAAATGCACAAACCAAGCAATCAACAGAAAAGCTGTAAATGTGTGTACAGTTGGCA
TGGTAGTATACAAAAAGATTGTAGTGGATCTAATTTTTAAGAAATTTTGCCTTTAAGTTATTTTACCTGTTTTTGTTTCTTGTTTTGAAAGATG
CGCATTCTAACCTGGAGGTCAATGTTATGTATTTATTTATTTATTTATTTGGTTCCCTTCCTATTCCAAGCTTCCATAGCTGCTGCCCTAGTTT
TCTTTCCTCCTTTCCTCCTCTGACTTGGGGACCTTTTGGGGGAGGGCTGCGACGCTTGCTCTGTTTGTGGGGTGACGGGACTCAGGCGGGACAG
TGCTGCAGCTCCCTGGCTTCTGTGGGGCCCCTCACCTACTTACCCAGGTGGGTCCCGGCTCTGTGGGTGATGGGAGGGGCCATTGCTGACTGTG
TATATAGGATAATTATGAAACACAGTTCTGGATGGTGTGCCTTCCAGATCCTCTCTGGGGCTGTGTTTTGAGCAGCAGGTAGCCTGCTGGTTTT
ATCTGAGTGAAATACTGTACAGGGGAATAAAAGAGATCTTATTTTTTTTTTATACTTGCGTTTGGAATAAAAACCCTTTGGCTTT
HUMAN SEQUENCE—CODING
ATGCTCTTGTCCAAAATCAACTCGCTTGCCCACCTGCGCGCCGCGCCCTGCAACGACCTGCACGCCACCAAGCTGGCGCCCGGCAAGGAGAAGG
AGCCCCTGGAGTCGCAGTACCAGGTGGGCCCGCTACTGGGCAGCGGCGGCTTCGGCTCGGTCTACTCAGGCATCCGCGTCTCCGACAACTTGCC
GGTGGCCATCAAACACGTGGAGAAGGACCGGATTTCCGACTGGGGAGAGCTGCCTAATGGCACTCGAGTGCCCATGGAAGTGGTCCTGCTGAAG
AAGGTGAGCTCGGGTTTCTCCGGCGTCATTAGGCTCCTGGACTGGTTCGAGAGGCCCGACAGTTTCGTCCTGATCCTGGAGAGGCCCGAGCCGG
TGCAAGATCTCTTCGACTTCATCACGGAAAGGGGAGCCCTGCAAGAGGAGCTGGCCCGCAGCTTCTTCTGGCAGGTGCTGGAGGCCGTGCGGCA
CTGCCACAACTGCGGGGTGCTACACCGCGACATCAAGGACGAAAACATCCTTATCGACCTCAATCGCGGCGAGCTCAAGCTCATCGACTTCGGG
TCGGGGGCGCTGCTCAAGGACACCGTCTACACGGACTTCGATGGGACCCGAGTGTATAGCCCTCCAGAGTGGATCCGCTACCATCGCTACCATG
GCAGGTCGGCGGCAGTCTGGTCCCTGGGGATCCTGCTGTATGATATGGTGTGTGGAGATATTCCTTTCGAGCATGACGAAGAGATCATCAGGGG
CCAGGTTTTCTTCAGGCAGAGGGTCTCTTCAGAATGTCAGCATCTCATTAGATGGTGCTTGGCCCTGAGACCATCAGATAGGCCAACCTTCGAA
GAAATCCAGAACCATCCATGGATGCAAGATGTTCTCCTGCCCCAGGAAACTGCTGAGATCCACCTCCACAGCCTGTCGCCGGGGCCCAGCAAAT
AG

TABLE 10
MOUSE NOMENCLATURE
ICSGNM Wnt3a
Celera mCG11700
HUMAN NOMENCLATURE
HGNC WNT3A
Celera hCG42253
MOUSE SEQUENCE—GENOMIC
TGAGCTGTTGATCACATGCTGCATTTAGAGAGAGTTCTGGGACTAAAGCTCAGAGAGAAGACACAGGCCCATGGCCAGAGAGGCCTTACCTATG
TGGGCCTTGCCACCTGTACCCAGGAGCACACCACTCCCTTTGGAAAGAGCACCCTTCTAACACCTATCATCAAAGTCAAGCAGACCCAGTCAGT
ACAGCTTCCTTTCTCTCCCCTGGCCTTGCTCTTTTGCCCTTTGCAGCCAACCAGTGGGTTGAGTTGAGTGGATGCCCGAACAGAGTCTTTCCAG
TGGACAATAGGCGCTGTACTGAAGCATGGTGGACGCGGGGACATGTTTGAATATTTCTGATACTTTCCCCCATCCCACCATGTGACAACCTTCC
AGCTCTGAGACCTAGAGGAACCTGGCCTCTGCCTCTACCTAGGTCTCCCTCCAGGGCTCCCCAGGTGACACCCTCGAGGTGTCTGATACATTAG
TGCTTCCACATTCCTTCACCAGCAGAACGTGCCGCTGCCGCCCAGAGAGGCTGGCAAACACATGGCCGCCGGCGCGGGAGCCTCCCGCCTGCCA
GGACTCGTTCAAAGTATGTGTCAGTAATCCTGCTGGCCCGGCGGGGCAGGACAGGCGCTGGCTGGGAAGGCTTCAGGCAGGGCCCTGTGCTGAC
TGGAGTAAGGACTTGGGACCCCTCCCCAACTCAGAAAGTGCTGGATCCTCAAGGAGGGCTTCCATTTTCTGGGGATGCTGTCCATTCGTGGGAA
GGCAGCGGATTGTTGGGGATCTCAGGAACACAGGTACCGTGGGGACAGCTCTCCATCTGAATGGTTCCTGGACAGCCCCATTCAAAAGAGGACA
GTGTGCTTTGTCCATGAACAGAGTGAGTCCCATACCCTGGGTCTCAGGTATGAGTGTGGAAGCCTGGTGATGTCACTGAGAGGCCCAGTGCTCC
AGAGAGAAAGGCCTGAGTGGGACCCACAGCATTCTGGCCCAGGACCCTTATCAACAGAGACTTCTCTACTCATGATCCTCCTTGTTCCCTCTGG
GCCTGGGCAGAGCCTTAGGAACCCTACCTGCTAGGATCCAACTCAGTGCTTACTGTGCCTGATTCCATGCCAGCAGCGGATAAGAGAAGTCAGG
CAAGCAAGGCACTTCCTGCACCAGATATAGATGCAGGGAGAGGTGGACAGACACAGTCCCAGACAGGCAGGTAGCGCTCGCACACACACACAAA
CACACACACACACACACANNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NGGGAGCCTTGGCCCAGGGAGGCCAGTGCTGGCCCAGGCTGGGCTGGCCTGCTGCCCATCTCCCAGTCCTGGACCAGCCTCCTTCCTCTACTGC
ATGTCACAGCCAGCGTCTCTGCTATATCCCTGGCCAGGTGCCCGGCCTTTCAACAGGAGCAAGGGAAGGAGGAGGGAGAAAAACCATTAGTGTT
TGCCGGCTGCAGGCGTGGCTCACGTGGCTGCAGCCACAGGATGAAACTCCTAGTCTAAGGATGGGGCTATGTTCCAAAACATAGCGAGCTCTAA
GTAGACGGCGATGCCTACCCCTTTAAAGACAAACTAGGAACAAGTAATAAAAAATTAACAGCATTAGTGAAAATGAAAGCAAGAGGAGTCTGAG
ATCTGATTGGCATAACACGGGCATTTTCACTGGTGTGGCTTGTTCTTGTTTGACACACACACACACACACACACACACACACACACACACGCTC
GCGCGCGTGCGCGCGCATGCTTTTGTAAGGATAAAAAGTTTCAGCCTCACTTTCTAAGGCATAGTTTAAACATCTGCATTTTGGGATCATTTTG
AGGGCGGCCTCAGCAGTTACCTGAGGTCTGAAGTGCTTAGGGTCCACTGCCTAACTTTGGCTGCCAGTCCTTCTGCTAGGACCCAAGAAAAGGC
TTGGGGTGGGGTGGGGTGGCTGGACCTTTCAGAGAAGGAATGGCAGGAGAACACTAATTTTATTTGGTGGGGGTTTGTCCAGAGGACCTTACAA
TGTGATGTCAAGTTGGACAGTTAAAATTCTAGCTGAAAGTAAGATTGTAGGCTTCTCCCAAGAGGCTGTCCTCCACCCTGGTTGGCAGAGAGAG
GGGAAATGGAATGAATTCCTCCCACCGTGAATCACAAAGGCCAGGTGCAGAGAAATGGGTGCAGCGAGGGTGTGCCTCCTCCCAGCTTCTCCCC
AGGCCCAGGCAGGGCCCTCCTGCCCTAGCTGTTTCCATCACCCCAAGGCAGGGTTCAGACCTTACGGTGACCTCACAACTGGAGGAGCTGGATG
TGGATTGGAAGTGACTGGTTACAGGGTCTTCCATTCTTAATGCTCTTCCTGGGCAAAAGATAAGGGCTCAGCCTAAGTCAGGACACGGGGTCCT
TTGAGTAACCTGAGGGTTCTCTGTCTTTGTCTGCACATGGGTTTACACACGTTAGAGGGATTCTGTGTGAAAGGGGAGGGAAGAGTTCATACAA
CACTCCTGGAGGCTCAGGGCGGGCCCAGGGGCTCAGGAAAGACTAGGTGCGTTCTGGATGGGATGAAGACCCCCGTTTGTCTGGCTGGTAGCTG
TCTCTTGGGCCTTGTAAGCAGGCAAGGGCGACTGGCTAACTGCCAGGGCTCAGGTCCCACTGGTCCCTGTCCCTTCTCCAACTCCTGATATCTC
TTGGGGGTTGGATCTGATAAATATTGGCCTTTAAGAGAGAGCAATGGCTCAAGAGGGCAGTGGAATTTCAGATGTCTAGGGGAAATTATGTCTA
TCATTAAGTTCTGGGAGAATCAGGTGGAGAAAGCAGAAAAGGCAGGTGGCAAAGGCCAGGTCAGGGCCCAGGAAGGCACTCAGGTGTTTCGAGT
TTGGACTCAAGCCTTAGCAAAATGCTTGTCTTTGGAGTATCTTTACAGTTATGGAATGTCAGGCTAGAAGAGACCTCAGATCAGCAGCTTAAAA
CTGTGTTTCTGTCACTATGCTCCAACCCTACGGTTGGTCACCTACGGTTCAGCTAGGGACACTGAGGCTCCAAGAGTCTCAGAGGGCAGACCAG
AATAGACCAGCACAGTGATGAGATGATGAGATGAGTCCTCACAGTTAAGATCATTGCTCTCATCTCCCTTGGTGACTCTCCCTTGATGGAAGTG
GTCCTGTAAGTGAAGCCTGCCTCTTTTGTAACACTGTCTCATTTTGATGTGCTAGCTGCACAGTTCGGCTTGATGCATTTCTCTGTATTGGACT
CATTCATGAATGTTACTTTGCTTAATACATTTTAAGACTATTACAGTGTATCAGGGCCAAGAGGATGGCTCAGTCAGTAGATTATGTATCTGTT
ACACAAGCACAAAGACCTGTGTTTGATCCTTAGGACACACATAAAAAAATTATCTGGTAGTATAGTTTTGAAATACCAGTGTTGAGGATGGAAA
GACAGGCAGGCCCCCGAAGTTCACTGACCAGCCAGCCTAGCCTAAAATAAGCTATAGGTTCCAATGAAAGACCCTATAACAAGACATAGTTCCT
AAGGAACAACATACAGGATTGATCCCTGCCCATCTAGTGCATGTGTTCACACCTGAACAGGAATGCGTGAGGGGGTGCCCTTGGGAGCCAGTGT
GCCTTGGGGACCATTGTTTTTGCCATATCAACCTGAAGTCTTGATATAGGCTCCACTCACCAAGCCTTGGTTTCTTGTCCCAGGTGGTCTTGGG
TCGCACCTGTCCTAGTTTGTCACATACAGGGTCTGTGAACATATCACTCGTGAGCTCCACCCAGGGCAACTGGAGCTGAGGGTGGGCAGATATG
GGAATGGGACAATGATGAGAGATCTGAAGGGACACACACAGCAAGAGATGATCTGTGGAAAGGTCCTGAGTAGCATCAGGAAGGGGCAGCTGTC
CACAGGCTAAGCCAACAAGCTTGTGGCATGTCGTTGTGTCATGTGCCCCTGGAGCTTGGACACAAGGTGCCTAAAAGGTGGCATCTCCCAGAGA
TGCCTTTTTGTTCTGGGTGTTGGGACTCAGGTAGGGGTAGAAGAATCCAGTGCAGTGAGAAGGACAGTGCTGTGCCCCTTGGTTCTTGTCAGAG
CAGAGCAGATGCCCCAGATTTAAGAACTTTTGCAGCTCAAGGTGTCAGCCAGACCAGGAAGCCACTTCTAACCTCACTCGCATTGTTGGGGCTC
ATTTTGTGTTTTTGTAGGACTGAGTTCTCCAGGACTGGGTGGCTGTGGCTTAGGGTAGCCCCTGAGAGTCCCTCTGTGGTCTCAGAGCCAGAAA
CTCGGAGGCAAACTCCATGCTTTGGATCCAACTTCTGAAAGCACACATGAGTAGCATAAACTTAGAACTCCAGACTCAGTGTATGCCCCAAAGT
CCTGACATTCTGGGCATAGTTTTGGACCTCAGAACTCTGGGAATACCTCAGAATTTGAGGTGCTGCTAGATTCTGGGCTTGGAGAGGCTTCTGG
GGATAGGGGATGGAGTCAGGCAGCTGTGTGTGCTAGAGAGCAGCTGTACTCAGCCTAAGCTGTAGAAACCTGGTGGGCATAGGGACCGCTGAAT
CATACTCCTGTGCAGTGTCTCAGTGCTAATCTCGTGCCCATGCTCCCAATGGGACAAGAAATAACATTCTCACCCCCAGCTCTGCTCATTGCCA
TGGGACCTCACCCTGGATCACAAAAGGAGATGACACGCTGCTGTTACAGCCACATCACTGAGGGAAGACAGCTATCATCATAGTGGGTGAGAAC
TGGAGAAGAAGTTACAGATGCTGTTGCTGTTTAGCGTAGCCTATAGAAGTCTAGCACACACAATAGCAGGACCTCCCCCCGCCCCTAAAAAATT
CCACCTTGCTCGCCTTCCTATAGCTCAGCTGATTTGGTTCCTCATAGCTGTGCTGTCTCCACTTTCCTTCTGGGCTGTTTTGTTTAGTTTTTCA
TTTTAAATATATTTTATTTATTTATTGTATGTGTTCATTTGCACTTGTGCACAGGCCACAGCATGCTTGGGGTGGGGGGTTGGAGGACAACTTC
AGGAATCTGTTCTCTCCGTCCACAGTGTAGATTCTAGGGAATCAATGATGTTTGTCAGGCTTGGCAATAGGCACCTTTAGTCACTGAAATCTCT
CTCCCACCCTGGAATGTGTCTTGATGACTGTACAAAGGGCTCCTGGCACTCACCTTGGAGACAGTGTCCCCTCATTCTGATGTTAAGAAAGAGT
AAACCAGTACATACTTGTGGTCCCCGGTGAGGGAGGTTGAGTCTAAAGAATCATGAGTTTGAGGCTAGTCTGAGCTACCTAGCCCGGGCTAGCC
TGAGCTAGGCTGAGCTTGTTGCTTAGTAGTAGTTGCCTGGCTGGGGCAAGTCTCATTAGTGGAGAAGGAGGAGGAGGGAGAGAGCCAGGAAATG
GTAAGTTAGCCAGAAGTTTAGGAGAACACTTGAGAAAGTCCTGGGGAGGGTCAGAAATTCATTGGCTACATGAAGAATTAGAGACTGGGATATA
TGGGACTCTGTCTCAAAAACAGAAAAATAAATAAATGAATAAATGTTTGACTTTGTACAGCTTTCCTTCTGTATTCTGTAGGCACAGGCTTAGC
CAGCTGAAAATTGAACATGTTTGGAAAAACTGGGCATGGTAGCACATGCCTTCAATTCCAGCACTCTGGAGGAGGCAGAGGCAGGTGGATCTCT
GTGAGTTTGAGGCTAGCCTGGTCTAGAGAGTGAGTTCCAGGGTGGAGTTTAGTTAGTAGAATGCTTGCCTCACACACACTTGGGAAGTAGAGAC
AGGGGGAGGAGGATTTTAAGGTCATCTTTAGCAACTTAGCAAACTGGAAGTTAGTCTGCACTACATGAGAGACAGACAGGCAGGCAGGCAGACA
GACAGACAGAACTAAACAAACTAATACAAACCTAAGCTGACCATCACAGCCCAGCTGAAGTCCAGAAGTTTATCGAACCTCCTCAACCCTGCCT
GCCCGTCTCCATCGTATGACAGCCACCCTGTTCAGTCTTCAGCATTCCTTCCCACAAGGCCTCTGTGCCTGGGCTTTCCTGATGGTCCAGCTCT
CAGGGAACAAGAAATGATCCAGGCTGCGATTCAAGGCACTTCAGAGGCTCTTGGGTCCAGAGAATGTTCTCTTCCACCTTTCTAAGTCCATGAG
AGAGAGCAGGAGGGATCCTGCTGGTGCTGCCCACACCCTGGCCTCCTAGCTAGGGGAGGTGGCGAGCAGGGACCTGCCCAGAGGAAGGGAAGCA
GAGAAGGCTGACTCAGCATCTCCAGAGGAAGGGGGCTGGGGCGCTGCCATGGACCTGGGAAGTCCCTTCTCTGCCTTCCTTGGGGGCCACTCCC
AGCAAGGTGGGTGGGGAGCTGAGGTCTGACCACTCCATGCCATTGGAAGGAGCCCCCTCTTCAGCAGCATCCTCTTCTTCCGAACCCACATCAT
CAGCACCCAGGATGAGTGCTGGTTTCTGGCATCTAACAAAGGAGACCACCTACTCTTGGGAACTGGCCACTGGCCTAGGCAGTTCCTCCTGGGA
AGACAGAAATGGCCAGGTTCTGGCCAGGTTCTCTTTTTCTGCTATTTGAGAATGGTCATGCTTTCCATTGTCTCTAAGTCTCCTAGTTAACTGC
ATTCACAGAGTGAAGATTGTCTTCTTTTTGGATGGATAGACAGATGCCCTTACCCTCTTTCTGGGAGCTATGAATGTGGCTCAGTGGGTAGAAT
GAATGCCTAGCATCTGTGAAGTCCTATGTTTGATCCCTGGCACTGCAGAGGCAGATGGATCTCTGTGAATTTGAGGCCAGCCTGATCTACATAG
TGAGCTCCAAAACAGCCAGGGCTATGTAGAGAAAACTTGTTTCCAAAAAACCACAAGAGGCTGGGCGATGGTGGCACACACCTTTAATCCCAGC
ACTTGGGAGGCAGAGGCAGGCGGATTTCTGAGTTCGAGGCCAGTCTGGTCTACAAAGTGAGTTCCAGGACAGCTAGGGCTACACAGAGAAACTC
TGTCTCAAAAAACGAAAACAAACAAACAAACAAAAATAGAGGAGGAAGAGGAGGAGGAGAAGGAAGACGAGGAGGAAAGGAGTAAGGTAGGGGG
AGGGGGGACATTGCCCAGGCCAAAGGAAAATCTTGCTTTGCCACAGGTAGGAGAGTCTTGGTGAGTGTGGGTGCAGTGGGCAGTGGGGTTCATG
GGTGCTGAAAGAAGGAGAGCTGAAGTACTTCTGGATGTGGGTAACAGGCAGAGAGCAGATGAGCCTGATGGGACATGATGGGACCTCTGGTTTT
AGGGTGACTAGCCAAAAGTGACAAGGTACAGCAGTGGCTTTCCACCTTGGTCTAGTTTCTTCCTCTCTACTCAGAGCACCTAGCCAGGCCCCTC
CTCTCTGCCATCCTTGAGGGCAAGGAGCACAGATCCCAGTCCACTCCATATGGACGTGTCTCAGCTCTCAAGCCAGGACCTTGGACAGTGATTT
CTTCATTTCCCACCAAGTGTCTGCTCTCCCCATACTCAGAAGTGCCTCTGTGGCCCATCTAGTGCCACCAGGGACACACAGACAGCCATGGTGG
TCTGTACCTAGAGAAGAAAGTGCTGATTCCGCCTGCAGGCAGGGAGGAGGGCATCTCTGAGCCAGACTAACCATGGAGGTCAGGGGCCTGGAGA
GGAGAGACAGCTGCTGAGAGTGGAAGCAATCCCCAGCCTACACAGGGCTGGACTTCTTATGAACGTGGGGCAAAGAGAGGAAGGATAAAGGAGC
AAAGAGCAGGCTCTGTCATGGTGTGCACAGGGATGCACGCTGCACCTAGGCCAGGCATGTGATGATGTGTGACGCAGGTCTCTGGAGTATTTCA
GGAGATAAGAGAGGCCAGCTCTATGGGAAGTAGACCACAAGGATGGATGTGCTGGGGACAGCCAAATCTCTGCATGTGAGGGTTCTACAGTCAC
AGACTTAGCTCCAGAAAAGTCCTAAGTGTTTGGAAAAGCCGTGTACAGCAACACAGACCTGTAATCCCAGGATGCTGAGACTACACAGTGAGAA
CCAGGTCAGCCAGTGGTACATAACAAGACCTTGTCTCAAAGAAAGGAAAGAGGAGAGATGGGTGTTGGAAGCCAGGCCCAGGGGCTCGTGCCTG
TAACTCCAGCCAGAGGCTCAGAAGATCAAGGTCGGCACTGGCTACACAGCAAGTTTGAAGCTAGTCTAGGCTACATGAGACCCTGTCTCAAAAA
TAAGAAAAACAAGGGCTGGAGAGATGACTCAGCAGTTAAAGATCGCTAACTGCTCTTCCAGAGGTCCTGAGTTCAGTTCCAGGCAACCACATGG
TGGCTCACAACCATCTGTAATGGGATCTGATGCCCTCTTCTGGTGTGTCTGAAGAGAGCAATGGTGTACTCATGTACATAAAACAAATAAGTAA
ATCTCTTTAAAAAAAATAAGAAAAACATGAAAATGAACATGGTGGGGGGAGAGGGAGAGAGAGAAACACTATTTGGGAAGGTTGCTGCATGTGT
ACTGACCGACTGACCGTGTGTGGGCTTCCGGTGCTGGCTTTCCCTGCAGTGTAGTGTGACAGTCTGTGGCCTTAGAATCCTTGTCATCTAGGTT
TGGCATGGACAGAAAGGTGTGTGGACTATGTGCAAATACCATGCTACTTAATACAAAAGATCCAAATGTCTGCTGTGAGCGTGTGGGTACGTGT
GTGTGTGTGTAGGCCAAAAGTTGATGTTGGATGTCTTCCGTCACTCCCCACTTTGATTTTCGGTACAGGGTCTGTCACTGAACCCAGAACTCCC
CTGGCTGCCTTTGCTCCCCACCTCAGTGCTATGGCTGCAGACATTTGCCAACCTGGTTTTTACATGGGTGCTAAGGATTGTATCTCAGGTCCTC
ACACTCACCTGGCAGGCATTTGACCAACACCTTCCTCCCCATCCCCTCCTCACATGTTTTTTTAGAGGACATTTGCTTCTCCCCCTCCCCCACG
CCTGAGTTGGCTAGTGCCCCACTCCACTGAGACACCTCAGCAGCCATCCTCATCCTATGTTCTCCGTCAGTGTCTGTATCCTCCAATGCCTATA
GAGGTCTTAGCCACAAGCTGAGGGTCCCAGGAATGCAGGTCTGGTTCTTACACACCGTGAGTCATGACAGTGTGACATGTGGTCTACATCCTGG
GTACATGGTCAACATGCTGTCTCTAGGTGCCCTTCCTGTCGGAAGCAGCTCCGGGCCGATCACATGCACATAGCCCAGAGTCTGTCCGTTTCCA
GAGATGGTCTCAATCACCTCCTTGTCTTTCCACCAGGTCCTTGGCTGTGGGACCCCAGTACTCCTCTCTGAGCACTCAGCCCATTCTCTGTGCC
AGCATCCCAGGCCTGGTGCCCAAGCAGCTGCGCTTCTGCAGGAACTACGTGGAGATCATGCCCAGCGTGGCTGAGGGTGTCAAAGCGGGCATCC
AGGAGTGCCAGCACCAGTTCCGAGGCCCGGCGTTGGAACTGCACCACCGTCAGCAACAGCCTGGCCATCTTTGGCCCTGTTCTGGACAAAGGTA
CCCTGGGGTGGTTAGGGTCATCTCTATATGGACATCATAAGGCCTTCCAAACTCTAACCTGGCTGGGCTTGCTCCTCCCGACCAATGGAGGGCC
TTGGAGAAGAAAGGGGGAGGGGAAGGGAACCGTAAGCCCACCAGGCGGGCGGGAGAGGCTTGGAGTATGTTGGGGAGGTGGTTCACCAGCCAGC
ACCCCTGGGCTGGTTTGGAAAGGAGAGACCGGCAGGAGAGCTAGGTGTTAGTTAGCTGCTGGCAAAGAGCAAATTATCCTAAAGTGTAGTGGTT
AAGGCAGTAATTACCATGTGTTGACCTCCTGCTGCCGGGGGTCAGGGGCTGGGTTGGCTCTGCTTCCCATCCACAGGGAGGGAGGTCCATGGGA
GGCTGTCTCAGCACCAGTTGCTAGAGAGGGCTCTGAAGTGGGGCAAAGGCTTCTTTCTGTGGTCTCTGGGACCCAGGTCTGAGACAGAGAGGGA
GACAGAGATAGAGGAGGCAGAATCAGAGAGGCAGAGAAATAGAAGAGAGACAAAGATGGCAGAGACTGGGTGGGGGGGAGGGGGCCGAAAGGAG
AGAGAACACACACCAAATGAAAGCTTTCATGCCTTGAACAAGAAGGGAAAATCATATAATTTAAATCACTTTAGAACATACAATCTCAGCACAC
TTAGCACACCCACAGAATTGCGACACTACCACTCCTAATTCCAAAATATTTTCATCCTCTCGAAAGGAAATCCCAAACCCATTATGCAGTGCCC
CTGGCAACCACTGGTTTGCTTCCTACCCCTGTGGTTTTCTTATTCTGGACATTTCCTCTCTGTGTAATTATGCACTCTGTGGCCCTTTGTAGCT
GGCTCCTTTCACTGAGTGTGAGGTTTACAAAGTTCATCTATGTTGCCACACATATCAATGCTTCATTCCTTTTGATGACTGAATACTATTCCAT
TTCAGTGTGTGTGTGTGTGTGTGTGCACGTACTCGTGTGCACCTTAAATTTTACATGTCAAAAAAAGAATTTTATATGTAAAAGATGATGCCAT
CTGTGGATTTCATTTCTTTTATTGGTATATGTTTATTGTATTGGGTTTTATGTATATCTTCATACACATATAGAATGTATTTTGACCTGACTTA
ATTCTGCTGCCCAGGACATCAGTCCCCTGTGGGACCAAAGTGGAGAGAGTGGGTCAAAACTCTAAGCAGTGGATGTCCGTGTTTCCTGAGTCAC
CCAGCCTTTGTGTTTGCTGCAGAGGTCAAAATACAGCACCTGTGGCTTTGCAAGCCTGGCTAGGAAGCATGGACACTGAACTGTCCCCAAGGAG
AACAGCATTGTGGAGAAGAGGCTGGGGCTGGGTTCCTGCAGTGGTCTTCCTGTGACCCTGAAGGTGTTTAAATTCTTTCTTCTGGTGAGGTCAA
AGCAGATGACCTCATGAGCCTTTGGGGTGGAAGTAACTCTGTTCCCTGCAGTCTGACCCTCCCCTGACTTTCACTTCCAGCAGTCCACTGTCGG
CATGGCTCCACCCAGTCCCATCAGCCTCCATTCACAAGCCAGCTCTTCACTTCATATAATGACATCATGCCACCAGTCCTGACCCTATGGTTCC
TCTGACCCCCTTTCACCTAACGTCATGACCCACCATTGATTGTCCTTCCTGGTGGCATTCACCAGCTCACTTCCAGGGGTGTTAGCGTCCCTAT
CTATAAAATGGGAAAGTAATTGCAGCTTGCAACTTGAGAGCTGTGAGTCAGGCTAATTACACAGTTGCTCCCCACAGAACAGCACTGCTGTATA
CAGGGGAGGATCAAGTCCCTAGGCCGGTACAGTTTACCAAGCATGCCTTATAGTATGTGCACGGGTGGTCCATCCTGTGTCTTCATGGGAATGT
GATGTGTGTACGTGCAGCAGTAAAAATGCTAGTGTGTACACATGTTTTCCTACATGACCACATATGAGCAGCACACGTGTATTCTGTTGCTTTT
TGTTTTTGTTTTTGTCTATTTGTGTTTTGTTGCTATTACAATGTACCTGATACTAATAATTTGAAGAGGGAAGATCGATTTTGGCTCTTGGTTT
CCGAGGGTTCAGCCCATGGTTAGAAGACTTCACTGCCTTAGGCAAGGCAGTCACAAGGTAACTGGAATGTGTTACCAAAGGTTTCTTCCTTCAC
TGGAACCAGAAAGCACAGAAAGCAACAGGAAGGAGCCAGAGGTGAATATCTTTCTAGGACATACCCCAAGCACGCTTCTACCTTCTTCCCCCAT
GAGCTCCCTGTCTCTTAATGATACCACGATATCCTCACTCTGTCAAGTCTGCTCATTAGTTCCAAGCTCAGGTTCCAATCACCTCAACAGTCCT
TCCCAGACAACACTCCATTTCAAAACATAGCAGCACGTGTGGTTATGGTATGTACATACGTGTGCACACATGGCTTCAGAGTGGGGCTGCACTC
ACATGTGAATCTGTGTGCACAGGTGTGGGAGGAGCTGAGAGTTCTACATCTTCATCTGAAGGCTGTCAGCAGAATACTCACTTCCAGGCAGCTA
GGACAAGGGTCTTAAATCCACGCCCACAGTGACACACCTACTCCAACAAGGCCGGCCATGCCTTCTAATAGTGTCACTGCCCAGGCCAAGCATA
TTCAAACCACGACAGTTGAGGAGCCTTTAGAGATCCTGAGAAGCTGGCCTTTGAAGGTTTCTGCCTGAAGTCTGTGCTGCAGTGAACAAGCCTG
ACTCCCATTACCACAGACAGAGCCCCTCCAACCTCCTGCTTCTCTGTGATTGACTGAAATGAGCCAAACATTACTTCTGTTGGGTATTTTATCA
CAGCAATTATAACTAATACATCCACCTCCATGGGGGAGTCTGTGGGAGCATGCACACACACCACCTCCCTCACAATGCTAAAGTGAGTCTCTCA
CAGGAGCAAGGAGGGCACCCCATGATTCGCCAACTATGTTTAGAGCTGAGGAAGGGGCAGGCTTTTATATCCTGTCATGCTAACAGAGCGGGTA
GCGCTCAGTTTCTCCTCTTCACCAGCACTCGGTATTGTTATTTTCTTGCAGCTGTCTTCATGGGCGTGAGCATTTTTTCTTGAATTTATCAGTT
ATGTGTGCCCTTTATAAAAAAATCTATCTATTCAGTTCTTCTATTCATGTTTTAATAAGGTTATTTCTATTTAGTTTGAGTTTTGATTATTTGT
GTTTTCCTTAATCATAGTTTTAGTTTCTCTCTCTCTCTCTCTCTCTCTCTCTCTCTCTCTGTGTGTGTGTGTGTATGTGAATGCTTATGTCTGC
CTTGGTGAATATGTGAAAGTCAGACAGTCCGAATCTGGTTCTCTTCCTAACCTGTGGATCCCAGGGATTGAACTCAGATCGAATTGGGTTCANN
NNNNNNNNNNNNNNNNNNCAAGCCTCCACAACATGGAGACAAAAACATGCAGACATTTAAAGTATTTTTTTATTTTTACTTTTAAAAAATGTAA
AAATTCTTTTAGTGTATAGCCTGTGTATGGTTTAGTGTACCTGTGTGAAGGTGCCCTTGGAGGCCAAACAGGGTCCAGACCCTCCCTGAGCTGG
AGTTACTGGCAGTTGTAGCTGTCCAGTATGGGTGCTGGGAACTGCACTTGGTCCTCTGCGAGAGCAGCCCATCTATCAACTGCTGACTGGTCTC
TCTAGCCTCCTACAGGAGTCTTTAACTTTGATGACAAGTCCCCAACTCTCTCTTTGGCTTTTTAGCGCTAAGAAAATCTGGCTGAACCCAAGGT
CACAGATGTTTGCTCCTCCCTGAGCCCCCCACGCCCGAGACAGGGTTTTTCTGTGTAGCTCTGTCTGTCCTAGAACTCACTTTGTAGACCAGGC
TGGCGTCGAACTCACAAAGATCCACCTGCCTCTGCCCCTCGAGTGATGGGATTAAAGCGGTGCACCACCATATCAGCTACAGAAAGCCTGCTTT
TTCTGTTTCCTCGAAAGAGTCTTATAGCTTCAAGCCAGAAGTGATGGCATGTGGCTCTATTCCTACTCAGCAGTCAGAAACCTGAGCTCTATAC
ATTTGAGAACAACCTGGTCTATATCCTGGTCTGTATGGTGACTTCCAGACCAGTCAGGGCTCAGCTCACAGTGCTTTTACATCAAAAAAAAAAA
AAAAGAAAAGTCCTATAGCTTTTAATCTGACATTAGGTCTATGATTTTGTTTTAATATTTTGATTAAATTTATATTTTGTGTGTATGGGTGTTT
GCATACATGTATGTTTATGTACCACATGCCTGGTGCTCACAGAGGTTAGAAGATCCCCTGGGACTGGAGTTATATACAGTTGTTGCCATGTGGC
TGCTGAAAATTGAACCCAGGTGCTCTGGGAGATCAGCCAGTGTTTTTAACCCCTGAACCATCTCTCCAGCCCTGCTTGATATATATATATATAT
TATGTTATTTTTATTTGTGGTATGAGAATGGGATCCAACTTCATCTTTTTTTCTTTTTTAACTTTGGCTTGTTTGTTTCTTTTTTCAAAATAAA
GTTTCTTTGTGAAGACCCGGTCGTCCTGGAACTCACCATGTAGACCAGGCTGGACTCCAACTTAAAAGAGATCCATCTGCCTCTGCCACGTGCT
GGAATTTAAAGCTTGTACCACCACCACCCACCTCCAACTTCATTCTTTTTTTTATTTTCATTTTTATTTTTTTCTTTTTAGTATAGAATAGAGT
TTATTTAGGGCATGGGGAGGGGAGCTAAGAGAGTAGTAGAGGCAGAGAAAGGCGGAGAGAAGGAGAGAGTAGAGAAGTGGAGGCCAGCCATGAC
CACGTGGAGAGAGGGGGGACAAGGGGACAAGAGGCAAGAGCATAAGCAAGCAAAGCTCCAACTTCATTCTTTACTGTGTTGAAATGCAGTTATT
CAAACAGCGTTTGTTATAAAGAGTATTGTTCCTTCTTGAATGGCCTTAGCTCACTCATTGAAAATCACCTGGCTTTGTATGTTTGCATTGATGA
ACATATAGGCTGTTTGCTCCATTCCACTGGTCACTGAGTATTATCATAGCAGTGTCATGCAGTTTTCCTGTCTATAGCTTTGTCCTGTGTTTTT
TTTTTAAACTCAAAGTTGCTTTGGTATCTGAGGCCGTTTATGTATGTTATCATATGTATGTTAGGATCCACTGGTAATGTCCGTTTTTTTAGAC
TTATTATTTTTAGTGTGTACGAGTATTTTGCCTACATGTGTGTATGTGTACCATGTGTGTTCTTGGTGCTCACAGAGATCAAAAGAGAACATTG
GATCTGCTGGAACTAGAGTTATAGATAGTGGTGAGCTGCCATGTGGTACTGGAAACTGAACCCAGGTCCTCTGGAAAAGCAGCCAGTGCTCTCG
ATTCCTAAACCATCTCTTCAGCTCCCAGTTGGATAAAATCTTGGAGATCTTAGGGACTGTGTGGGAGAAGGGGGGGGGGAGAAGAAGGGGGAAG
AAGGAGAAGGGGAAGGAAGAGGGAGAGGAGGAAGAAGAGGAGGAGGAAGATGAGGAGGAAGGGAAAGAGGAGGAAGAGAAAGAAAAGGAGGAGG
AAGAGGAGGAAGAGGAGGGAGAGGAGAAAGGGGAGAGGAGAAGGAGGAAGAGGAAGCAGGAGAAAGAAGAGAGGAGAAGGAGGAAGAGGNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNTCACAGCCCTACTGCAT
CAGTGACACTTGGCGGCTCCCATCTCTGAGTCTTGTACATTGTTTTCTAAATTGTTCCCGAGCATTTCTTCCATTGTTATCTACCGTGACATTC
TTTTCTTTTTAAAAATTAATTAATTTTATGTCCCTGAGTGCTTTTGGGCACCACTGTCATGCCTAGTGCCAAGGAGATCAGAAGAAGGTGTCGG
TTTTCCCGGAACTGGAGTTACAGATAGTTGTGAACTGCCACGTGGGTGCTGGGAACTGAACCTGGGTCCTCTCCAGGAGCAACTAGTCTCTCTC
CTGGGATAACCATCTCTTAGCCCCATCTTGCTTTTCTTACTCTGTAGACCAGGCTAACTTCTAACTCACAGAGATCACCTGCCTCTGTCATCCA
AGTGCTGGGATTAAAGGAGCACACAAGGCTTCCTGCTCATCATTGTTTTATTTCCGTTTTGTATTGTGTTTGGATTGTTTGTTGCTAGGAGGCA
GAGATTTGTTGAGCTCAATGAGGTGCAGTACAATTGACCAGCTGCTCCGTGACCTGGCTGAAGCTATTGATAGGCTCCAACAGGTTGTTTGTTG
TCTACTATTGGTTCCTTGGGCTGCTCTACGCATAGGGTGATCTCACCTCTTCCTCCCATGCTGGATGCTCTTTATTCCTTTACTTGATATCCAG
GGTGAGAGCATCCATATTTTCCCAACCTTGAAGTGACTGCTTTCTGCCTTCCCCATAGACAGTGATAGCTGTGGGTCCATCCAGGTTTTAATTT
TGTAGATTCAGAAGTAAGAACCCTTTACTCGTGTATAAGTAGATAAAGGATTTTTTTAAAAAAATATTTTATTTATTTTTATTTTGTGCATATG
GGTGTTTTGCCTATATATACACCTATGTGCCACATTCATGCAGTGTTCACAGTGTCCAGAAGAGGGCGTTGAGTCCCTGGAGCTAGCGTTAAAG
ATGGTTGTGAGACACCATGTGGGTGCTGGGAATAGAACCTGGGGCCTCTGGAAGAGAGGCCAGTGCTCTTTAATGGCTGAGACATCGCTCTAGC
CCTTAAAGTTAGCAGTGTTTTAAATTTTATTTGTTTTACACACACACACTCATGCACTCACACATGTGCGCACACACATGATTGTGTGCACATG
TGTGAACATGTATGCAAGTGTCTATGGAGACCAGAAAGGATCATTGGATCCTCTGGAACTGGAGTTACAGATGGTCATGAAGAGCCATTTGGGT
GTTGGATCTTCTACAAGAGAAATACACACTCTTACTGGCTAAGCTATCTTTCTAGCCCTGAGAGTTTGTTTTCAATAATCCATTCTTTTGATCT
TTTGGAATTATACCAACATTTTAATGACAATCTATTATCAAAAGGTTTATAGATGGGGCATAGTGGTATACATCTTTAATCCCAGTACTTGGAA
GTCAGAAATAGATGGATCTTTGTGAGTTCGAGGCCAGTCTGGTCTACATAGCCATGTCTCCAAAATAAAACAAGTTCTCCAAATTCATGAGCAC
ACAACTCAACTTCATTTTATTTATTTGTTTCCTTGCTTAGAGATAGCATCTTACTATGTTGTCTTGGTTCTTATTGAATGCCCCAGTTCAATGA
ATCCTACTGCCTCAACCCTCTGAGTAGCTGAGGCTACAGGCATGGGCCACCCATTCATTTTCCTTTCTTAAGTTCCACGGCAAACATAACTGGA
GGTGAGCAGAATTTCACAAGGTGCTGTAACTAGCGGCAAGCACCTCTTTCTTGATTTCTAGGACAAATTCTCTGGGGCTAAGCACATTCTAGAA
AGATCGAGAAATCGTCCAGAACCAGAGCTGGGACACGAGAGGCTCTCCAGTGGATATGTATATGATGGCAAGCGCTCCATCCCACGCTGCAGAT
GCACACAACCTATGTGCTTTCAGTCAGCACTCTGCTGGGCAGCCCCGGTGCTCTAGCCTATGCATTTTGCTCTGTGAATGTGTCAGATGACTGA
AAGACAAGGGGCTGGTGCCTGGGAATATCTTGGAAGGCTTCCTTGGGGAAGCGGCTCCAAACTCTTTGGGCAGACAGTAGTATGGTTGTGGGGA
GCCCAGGGGTGAGAGAAAAAGGAGCAGGGGAAGGCAGTGGGGCTCAGCAGTCTGACTAAACTGGAGCCTGAGGCACCGTGGAGGAAGAGAGAGG
ATGGCTGTGCCTGAGGCTAGAGCCCCGTCACATAATTTTGGGCGAGGTTAGGGGAGCTGCAGAATGTTGAGGGTCTCCCAGTATGCTCTTGGGC
AAACACAGGCTGTCTAGGGACAAGCAGCTGCCACAGGTCCTGCCCTAGAGAGCAGATAGTCACATCAGCCTTGGGGCTGAGGGCCGCGCCTGCC
CACATTCCTCTGCCTACACCCTTGCCTCCTAGAAGCCACCTGCAGGACAGAGAGAACCTAGGCTGTCTGGCCTCAGCATCCCATCCCTCTTTCC
CATGGCAGCACTGGAGGATTTCCTGCCAGACGGCAGCCTGCACCCCTGCAACTGTCCTCCCTGCAAACATGACTTAGGCCTCTCTCAGTTTGCT
GCTCCCTTCTTCACTCTGTCCCCTCCGGTCCCCAAAAGCCCCATCCTCCTCCTCCTGCTTATGCTCCCCTCCTCAGTCCTTGTTCTCCTTCTCT
TCTGGTGGCCAGGGGAGAAAGGAGCCCTCCGTGTGCCCCCAGAAAGGCTGGCTCCAGAAGTCCTTGTGCCCCCCACCCCCTGCTGCCTGCTGGC
CTCGAAGTAATCAATCTGATTGGATTAGGGTCAGGGCAACCCTAATCATGTCAAAGGTTACCCTAGGCTTTGTGGCAAGGACCACAGGGGTGCC
TGGCCACCCCTGCCGAGGTGGCCTCTGTTGAACAGAGTTTAACATCTACCAGCCAGCTGGGAGGAAGGGAGCCAGCCTCTTGCGTGCATGTGTG
CATGTATGTGAGCTTGTGCAGCCATGTGTTATAGAAAAGCGTGTGTGCATGTGTTTTATATATGCATGTGCGCATGAATGTCTTACGTGGGCCT
GCGCACTTGTGAATATGAATGTGTTTGTGCCCATGTTAGTGAATATGTCTGTCCGAATGTGTGTCATGCATGGGCATATTTCAGTGTGCATTTG
TGTGGATGTGCCTTACGTGGGTATGTGTGTAAGTATATGACTGCATGTGCTGTGTATGGATATGTGGAGATGTGTGCACGTGTCAGTGCACATA
TACATTTGTGCACCTGTGTATGTACTCACAGGTATTGATTATATATGTATAGATGTGTGAATGGTAGCTACACACAAATGCTCGTACATGTGTG
CTCAGGTGTCTGTCTCTGTGTGGGTGTGACCATGTACACGGTACAGGCGTGTGTGCATATGTATATGCATTGAGCATGCATGTGTGAATGGGTG
CCTGTGTGTATACAAGGCAAGTGTTTGCTCATATCTAATCCCAAAACCATCTGCCCTGTGTCAGCCTAAAACTCAGGATGCTAACCTGAGCGTG
AGGAAGCTCTAGCCCTTGAATGCAGAACGAGGTCCTAGAGGGCTCTGCTCTTGCACCCAAAAGTAGCAGGGAGCGACCTACCTACCTACTCGGG
ATCAGAAAGCATTTGAAGAATTTTGAGTACAGGATGATTGTGAACCGTTTATATCCCTTGAGGCAAGCCTCAGACACTTGGAGGCAGGTACTAG
GTGACTCCCCTGTATTGACCTCTGGCAAGATGAAATGGGTGGGGTGGTTGCAGGGCCAGCCTGGGCCAGAGGTAGGGGTCACAGGACGAAGGTC
AATAAAACAGCTGGTCAGGCTGGAAGGGGACAAGTGGGAGGCTCACAGGATTGGGGGGTGGGGGCAGAGTCCAGGAAGCAGGTTATTAAACACT
ATGTGTCCTTGTGAGGGGACAGCTTTCTTTTGGCTGTGTGTGACATTTGCATGTAAGACATAAAGGCACATGGTCTGGAGCCCACAGCCATGGA
AACTGGGGTCTCTACCTGCCTGTGTACGGGGGTGGCTGTGCCTGGAGTCCTCAGAAGCTCCTAGCTGGCTTCCCTATGAGGGAGGAGCATCTTC
TACCCACCTTGCAACACTCCACCCCACTTGGGGGTGGTCTCCCCAAGTCCCTAAGGACTCCTGGGGGCTCACTGCTTGTACAGGCAACCTTTAT
TCACTGCGAAGTCTTCATTTGGGCCTTGCTGGCCTGGACAGTGTCTCCAGCCAGAGCATCTTACCTCGTGTTGGGTGTGGTCCCTTCCTCCAGC
CTGTGACATACCTCCAGGCCAGCCATACCCTCAACCCATAGTGGCTGGACTTTGTTGGGTCCCCCAAACCTCTGCCACAACTATTAGCAGAGTT
GGTTGCTTTTGAACATCAGACTCTCAGTATCTGCTGAACTCACCCCAAGCCCTACTTGGCTTCTGGGCAGGGAACAACAGCTTGGCCTCTTCCC
CTCCCCCAGGCAAAACCCCTTCCTGATGACTGGCCTCAGCTTCGTGGCTCCTCAGGGGGTGGTCCTCCACCTCTCTGTGTGCTCAGCCTATGGA
AGGATCGACTCCAATGAGACTAGCCTGCAAGACAGCCGGAGCTTAGGGGTTCCGGAGGGACAGAAAGGGCAGCTTTTTAGGGAAGCAGAGTCCC
CAAATCCAGGTTGCGACCCCTGACCCCCCCATCCGAAAGTGTTACCATTTACGATGCGTTGGGGGTGGGGGGGCTCCCCTGGCTCCGGGGGCCT
CTCCGGTATATCATTAACTCTCCGCTGTGGGAGGGGGGACCCCGGCCGATATCCCACTGCGCTCGGCTCGGAGCACAATGTCCCTGCGAGGACG
ACAAGATTAAGCACAAGGGAAAAGTGAAAATGCTGTTGGCGATTCACACGCCCTTCGGGGTGTTGAATTAAAGCGTCTGAAAGAGGATTTCCCC
TCGCGCCCGACGGCGCGGCGGGCAGCGCATACTTCCAGGTTGCTATGACTGGCCGGCGATCGCCTAATCACCGGCCAGGGGGAAATTCATATAA
AATATCGCTGGCCATTGTGGGGCTAACGCGGCGTGACAGTGTCGCACAAAGCCGGATTCATCAGCCCCAGCAGGTCAGCCAGGCGCCCGGCCGC
GCGGGCCCGGGCGGTGTCAGGGGCCGCGGCCACGGTGGGGCACCGGCGCATTCCGCTCTCAGGTGGCTCCGGGGCCTTTTGTGCCTCCGTGTGA
TTACCAAATGCCACCGCGACACCCCCTCCCGGCCGCCCGGCTCCTCGAGGCCAGGAAAAGGGCCTGGGGAAGGAGGGACGGTGGGCAGCCTTGG
AGGGGAGGCGCCAGGGCGGGGCCTGGGCTGCAAGCTACCAGCTGGGGCTGCTCGGTTCTCTCGTCTGCCCTCTGCCTGCTGTGCTGAACACGGG
TGGGGGAAGGGATGCCCACCCACTGAATGTGTTATGGAAGCACTTTCAGACATCGGGATGGGAATGGGATAGATAGATGGTTTGTCTTTCCTCA
GCGTGACAGGAGAGTGACACTCCCAAGGGAGGTGATGCCCCATGACCTGCAGGCTCTGGCCAGTCTGAGCAATTATGGCTATGCCATAGTTGGG
CCTTCTTCCCACCAGTGGTCGGATAGTGATCTCCCAGTTGGAGTCTGGAAATTGAGAGGACAGAGACCATCTGGAGGTTGTTGCCTGACAAACT
CAGCCCAGGCTGCCAACCCTAAGCCTTCTGTGGTCTCCAGGAGGGTGTGGTGGATGTAATGTGCAGACATAGGTAAGGGTGAACCTTCTGTCTG
CTGGGATTCCCTGGTTCTAGTCCTGTGCAGGGAAGCTTCAACAGAGATAGAGGGTCCTAAATAAACGCATACATAGCATATGATCCTAAAGGTT
CATGCACCAAACTTTGGTGCACCCAATTTCAAAAATAAATAAATAAATAAATAAATAAAAATATAAAANNNNNNNNNNNNNNNNNNNNCCAATT
TAAAAATAATAATAATAAAAATATAAAAATAAAAATAAAAATAAAAAGGTACTACTGGATTTAAAGGCACAGATTAACCTCAACTCATTAATAG
TAGGCAATTTCAATAAGCCCACACACACATGCACACACGGACACGCACACAGCTCGTACCTGGTTCTGGAGAGTAGACACCTTCCATATGATTC
ATTTCACTTCTAGTTCTGAGCCTAGTGCCTAGCTCATTATGGACTCTCATATTCTGGATAGGTGATGGACGGATGGACAGATGAATGGATGAGT
GGGTGGATGGATGATAAATGGGTAGTAGATGGATAATGAGTGCGTGATAGATGGACAGTAGACAGATAATTGAGTGGGTGGTATTTTGGTGGAG
GATGGGCGTATGATAAATAATGAACTAGATGAAGGGAAAGGAAGGAAGCCCAGATAGTGATACTTTCCTTAAACCCAGTAAGAGCGCAGCAAAG
CTGAGATTGTTTCTGCAGGGGGACTTTGCTCCCCACCTCCCAGGATCTGGGTGAAGAGACAGTGGCCTCAATTCATAATCCTCCCACGTCAGCC
TTCCTGGTGCTGGAATTACAGGCCTGAGCCATGACACCGTTTTTGGAATTTGCTTCTTTTATAGAACAACTTCTTCAGGTAGTTTCTTTTCTTT
ATCTTTACCTGTATCTGATAAATAGACAGTATCTCCAGCCCTCCAAAAGCTCTTCTTAGCTCTGCCCCCAGATGACTGGCCCAGATGGGCAACA
CTAGTCTGTAAGAGTCCTGTGCTCAGGCACCCACCCCCTCCTCATTTGATGATCAATAGCCATTTGAAGGGTCTGATGTCATGCACTTGTTTTC
TGGAGGGAGGTGTGCTCTTGGAAGCCCTCACCTTTATGCTGGCCATTTCTGCTGAGTCAACAGAAACTGAAAGGTGCTGTGTGTGCCAGGAACT
TTGCTGTGGTGTGCCCCATTCCCATTCACTCTCTGCAAAAGAGATAGCTCTGGGAGAGTGCTTCATAGCTCTGGTAGAGTGGTTCATTCACCAT
GGTTGTCCTAAAACCCTATGGGACTCTGATGGAAGCAAAGGGTTTCCTCTCTGAGAAAATGTGTTTGTTCCCAAGATCCACGTTTTGCCTCTGA
CTCCTGGGTAGACCCAATCTTGTTGGATTAAAATCCCTGCCATCCTAGGATGTCACCTAAACATTCTGGAGCCTTTCCTTTCTAGGTCTCCTGC
AGACATGTTCTCAGTTATTATTAAGTATCTTAAGTATAGCGAAGCATTGCAGGAATGGTGTGGAATCTCAGGTCCCTGTCTTCCTCTCCACACA
CACAGCCTTGCACACTGCTGTGGTACATGGTAATTGATCATGAAAGCCAACCTGACTTGGATAACCCAGGTCTTTGTGCCTCCATGTCCCTTGA
GTTCCACCATCCTTCCCTTTTGTACTGCCCAAATCATCACCATTTATAGGAAGGACCTTAATCTGGCTTTAGCAGCTGCCTCCAATTCATTTAA
GTAGAGACTATACATTTTTAGCAAGAAAACATTGAAAACGATACAATCTTTCCTAGGAGGTAGATGGTATCTATTCACTGCTAGGAATGTGAAC
TCCACTCCTTGCATAGTGTGTGATGGTGATATCTTGGGAGGTGGGGTTGGGGTGGGGAACTGCTTAAGACTTTTGGCTGCAGCTTGCCTCCATC
CTTCTTTTCCAAGCATGTTCCATTTCACATTCACCTAGCCAAATGTGGGTTTGCAGCCCACAGTTCCTCCCCTGGCTTCTCAGATGGAGTTTCT
GGACTACTTGGACTTTCTTACTTCTGGCCTTTATTTCTTCCCCAGCCTGTTGAAAGCCAGCTCCAGAACTTGCCCTGCCTTGACTTGATCGGGA
GCCCCCACCTCCTCATTTGACAGTAGAGGAAACTGAGGTTCAACAGAAAGGGAGGACTTCCCAGCCTGCTGGTGACAGAGCTGAGCAAGAATGA
ACTCTTGCCTACTTCAAGTTCTATGTTGTTCTCTCCTTCTCCCAAGGACCCTTCCCCTCGAGTGTGCCTACCAGCTCCAGGCTGTTTGCGTGTG
TCTTGATAAGCAGGGAGCCTGAAGGCCTTGTTCTGGGCAGGGCAAGGCCTAGAGTGCTGGGCCCCCTTATCTTTTGGAGCCAAGAAGGATGGGG
GAGGAGCGTTTGGGTGACACTTGAGCTGGATAAGGTAACCAAGGCTGCCTTGAGACTTGGAATGTGAGGGTGTAGGCAGAGAAGTAGACTTCTG
AGTCGCACCCAAGAGAAGGTAGCTCAAGGCCTCTGGCCCCAGACTCAGGTTCTATGCAGAAAGAAAGAAGACTGGAGTCTTAGGGGCACGAGGA
GGAGTGCCTTGGCTCCTTCAAAGATCAAATAGGGGTTAGAGATGTAGCTCAGTTGGAAGAATCTTTGCCTGGCATGGACAAAATCCCCAGGTTT
CATTTCCAACACTACGCAAAGCTAGGCGTGGTAGCACACAGCTGTAATGTTAACACCTGGTGGAGTTCAAGGTCCTTCTAGGCTACACAGTAAG
CTCCAGGCAAGCTTGGGCTGAAGTCCCTGCCAAAAAGAAAACAAGAAAGAAAAAAGAAAAATGACGGCTCGAAGGGGTAGCCTGTGCTCTGGAA
TGGAAATAAACAGTGAACAAGATGCAGATCCCACCCGTGGGGAGGATCTGGACTGGAAGTGGATTCCAGAAGACAAGTCTTCCCGAGGAATAGG
CACAGCTGACATGTTGGGTAGATAGTGCCCAGGACCTTGGCTGCCATGCTACAGAGTGAAGCTGGTAACACTGAGGCTGGGGTAGAGCTTGAGC
CCAGGGAAGAATGAAGGCTGAGTGTGACCAGAGGCCAGTGAGTGCTGAGAAGAGGAGGAAGACAAGGCTGGCGGCAGGAAACCTTTAGAGAACT
TTAGGAAGGCTGTGTATCACACTGGCTCTGGGCACGGAGCTGGGCCTGCAGTGGGTGGCTGTGTGCTTGGCCAGTGGAGGCTACTGAATACTGG
GTGACAGTTCAATTGAGAGCTTCTCTGTGGTTCTGGTAGTTCCTTGTCCATCGTAATGTGGGAAGAGTGGGAAGCAGCTGGACTCCTAGATACC
TGCAGAAGTTGTCCAGGCTCTGACAGTGCCCCAGGTTGCGGGCAGAACAGCTGGAGTCTGCTCAGAGATGAGAGTAAATGTGTGTTCTTTGATT
CTATGTGTGGACGCTGACTGAACTTTGTGGGTCAGTCTGAGTGTGAGTGTAGTTGTTTGCAGTTTCTCTGTCCCTGTTTGGGCTTTGGGACACA
CAGAGTCCCGAGAGTCTAACCTCTTAGATGCAGCTTGAGGAAATATGGAAAGCTAGTGTGTGCAGGAGGGGTGAAGAGGCCACAACTTTTTATG
AAAAGTCTCACAATCCCAAGAGGGTCACCCGGCCCAAGAAATCCTGCGCAGGCAGGCCTGCGAGGCTTTCTGTCTGGAATGGTTTCTAGCCGGG
TGTGTAAAATCTCTGCTAGTCAACAAGTCAGATTTGTATCCATCTTACTCAGAACACCGTGCTTATCATACAGCAAGCACTTGGCACACCTTTA
TCTGGTGTCAAGTAGACGAGGGAGTGTCTTCTCTTAGGAAGAAACTCCTATGCACAATTGGTGTGTGTGTGGTGGGGGGTGTGAATCAATGCAA
CCACTATAGAAATGTTTGTGGATTCTTAAAAAATGAAAAATGGAACTACCACAAGACTCTATTTCACTTCTGGACCTATATCCAGAGAACTCTA
TACTCTATCATAAGGATATTTGTACCTCCGTGTATAATGCTGCTTTATTCACTATAGCAAAGAATTGGAATTAACCTAGTTGTCCATTAGCAGA
TGAATCGATAGTGAACATTTTGCATATATATAAAAATGGATACTATTCATCTCTTGATTTATTTTTTGCTGCCTTTTGTAGTCAATATTATATT
TCGTTATCACAATATTATCATTTCAAGATCCGTCCACATGATTTGCCCTCTCAATCTGAGGCCCCTGGTCTAGACCTACAGTGGATGCCTACTG
CCGGGAATAATGCAAAGTCTGGACTGTTGTTTTTCCTGTTTATGCAGACCGGTGATAAAATATCAGATATGTTAGACACAGTCAGATTTAATGA
CCAATCTCCAAACAGAACAGTTACAGTACAGCTGTGGTANNNNNNNNNNNNNNNNNNNNCTAGTCTACTTGGCTTGCAGCAAGCTAGGTAGTTC
TGGGACCTTAATGTAGTCCTCCTTGGGACTCTCCAGGAAGACAGAAGGGATAGAGTGAATCACTCTCCATACAAAAAGGATTTAGTAGAGTGGC
TTACAAGCTGTGATCTGGATAGTACAATGGCTGTCTTCCAACAGAAAGCTCAAGAATCCGGTGTTGTCCATAGACTGGATGTCTCAGCAGCTCT
TCAGTATACACTGGAATCCCAAGAAATGGGCTCTAAACTCAGTGAAGGAATAAACTTACCAGGGAGAGTGAGGGCAAGCAGGCCAAAAGCAGAG
GTGCTTCCATGTCCTTTGCATAGGCTGTCACCAGCAGGCATGGCCCAGATTTAGGGTAGGTTTTCTGACCTCAAATGATCCAATCAAGAAAAAT
CCCTCTCAGTGTGTGCTTTGCACACCGTTGATCCCATCGCTCAGGAGGCAGAGACAGGCAGATCTCTGTGTGACCTAAGGTAGCTTGGTCTACA
TAGTGAGTTGCAGGCCAGCCAGGGCTATGTAAAGGCTCTGTCTTAAAGCAAAAAAGCCAGACAATGTTGACACACACCTTTAATTCGAGCACTT
GGGAGGCAGAGACAGGCAGATCTCTGAGTTCAAGGACAACCAGAGCTACATAGAGAAACCCTGACTCAAAACAAAAACAAACAAATCCCTCACA
GACATACCCTGCAGGTTCCCCTTTAATTAACTCTAGATATACTCAAGTTGACAACCAAGAATAGCTGTCCTGGTTGGACTTGAACTCAAAGCAA
TCCTCTTGCCTCAGTTTTCCACATGCAGGTATTACAGGTGTGTAACATAAGTTCTGGTTTAAAAATCTCTCGTTACCTTAATTTTTAATGTTTA
TTTATAGATCTAATTTTATGTCAAAGATCTAATTTTCCTTTTGTTGTTCTGAGATGGGGTATCATGTAGCACAGGTTAGCCTTGAGCTGTCTCA
AGCTAAAGATGAACCTGAAGCTCTGCTCTTCAGCTTCCACCTCTGGAGTGATGGGATTTCATTATAGGCCTTCACAAGCTCACTTCAGGCAGGG
CTGGGGTTTGATCTCTATTCCTTTGGCATGTCTCTACCCATTGAGCCATATCCCTAGCACACTCATTTTATTTGTTTTTTTGAGACAGCCTCAT
GTAGCCCAAGCTGGCCTCTATGACCTTGAATTTTTTATCTTCCCGCTTCCTCCTCCCTGATGCTGGAATTACAGAGTGTACAGCTATGCCCCAG
TTCATGTGCTGCCAGGGATGTGAACACAGGGCTCCATTCATGCCAGGCAAGCACGCTAGCAGCTGAGCTGCACCCAAGGCCCTACTTCTGTTAG
CATAATTGTAAGTATTTTTAAGGTGCTGTGCAATATTTCAGTATAGGATTATGTTTTTTAAATGACCCAATAAGGGTTCTTAACATGTCCATCA
AGAACATTCAAAATCCTTTTCAAGTGATTTTTAAAAATACGATCACTTGTCCACTGTCCTCACATGACTGTTCAGTGGCACACCAAGACCCCTT
AACCATCATCTGAAATTCAGACCCACTGATCATCTTCCCTCCACCACCGTCCCAGGCCCTGCCTCCGTAAGTAACCCATTCCACTTCCTGCTAG
GAACGGAGTGCTTTTAGATTCCACACCTGAATTAGAACATGAAGTTCTTGTCTTCCTGTGCCTGGCTCATTCACTTATGTCCTCTGGGTCTGTC
AATCCTGCTGCAAGTGACAGGGTCTCATTCTTTTGTAGTGTAGCCTTGGCTGCCCTGGAACTCACTCTGGAGACCAGGCTGGCCTCAAACTCAG
AAATCCACCTACCTCTGCCTCCCAAGTGCTGGAATTAAAGGCATGCACCACCACTGCCTGGCTAGGGTCTCGTTCTTATTTATGACTAAATAAC
ATTTCGCTGTGCACACTACAGAGCTATCGTAACAAAGCACATACATACAGTGCTGGCATAAAGGGACACACACAAGCTGACGACTCAGGACAGC
AGTTCGAGAGCTAACTCCACATAGTCCCGCCCAATTGTTTTTTCAGCAAAAGGTATGAACGTACATTGGAGAAAAAAGTCTCTCTGACGAATGG
AGCTGGGAAATTGGATATCTCTATCTACGTAAGAACAAATTCAGTCCCTCCTGTTTCCTATGCACAAAACCCAACTCCGTGTAAGACCTGAAAC
TACTGGGACAAAAAGCATAGGCCTGGGCATGGTGATGATGCCTTTGATCCCTTTTGTGAGTTCGAGGCCAGGCTGGTCTGCATAGAGCATCCCA
GGCCAGCTTGAGAGATAGAATGATTCCCTACCTCAATAACAACAACAACAACAGCAACAACAACACACACACACACACACACACACACACACAC
ACACACACACACACACACACACACACTCACTCACTCACTTAGGAAAAACACTTGGGTCATTTGGGTCAATATTCTTCATAAAAGATCCTCAGAA
GAGCAGTGGTGAGACCCTGTGAGCACTGAGTTCTCCTCTCTTGGCCCTGGTTTTGGATTCCTGCCTTTTGGACTACAACCCTCCTCACCTGGCC
TCCATAACCCCCTATCTCCTTCCTCTGCAGCCACCCGGGAGTCAGCCTTTGTCCATGCCATCGCCTCCGCTGGAGTAGCTTTCGCAGTGACACG
CTCCTGTGCAGAGGGATCAGCTGCTATCTGTGGGTGCAGCAGCCGCCTCCAGGGCTCCCCAGGCGAGGGCTGGAAGTGGGGCGGCTGTAGTGAG
GACATTGAATTTGGAGGAATGGTCTCTCGGGAGTTTGCCGATGCCAGGGAGAACCGGCCGGATGCCCGCTCTGCCATGAACCGTCACAACAATG
AGGCTGGGCGCCAGGTATGTGCACAGAAGCCCAGGGCTGTAGAGCAAGGGCTGCTTTTTCACCGGGATGTCCAGTGGGGCAGGACAAGGGGTAG
AGGGGAGGTGCTCTTGGTGACCAGGCTGCTCTGCTTGCCTTCTATCAAGGGACATTGTGCCCAGCTGTACCTCAAGTGTCCAGTGTGCTCCCAG
AGAATGCCAGACTCACCTGTGGACATAGGGTTCCTGGAAGTAGACAGGTGGCAGAAGTAGCTGCAGAGCACATAGTCTCTCAGGATTCCGCGAT
GAGGAGATGTCCATGAGAGGACAAGACGTGAAGGGAGGAGAAGAGGGATGGAGGGCAGACACGTATAGTAGGTACAGATTGAACTGAGATAGAG
GGTTAGAGTGATAGAGATGATTGATACAGGCTACTGAAACAGACGAGAGGGTGAGAGAGGGGAGGGGAGGGGAGAGAGAGGCAGACAGACAGAC
AGACAGACAGAGAGAGAGGAGAGAGACAGAGACAGACAAATAGAGAGATAGAGAGATACAGAGAGATAGAAAGAGACAGTCAGGCAGACAGAGA
GAGAGAGAAGGGAGACAGAGATAGACAGAAAGACAGAGACAGACACAGAGAGACAGACAAAGATATAGGAAGACAGAGAGAGAGGAGGGAGAGA
GACAGAGACAGAAAGACAGACAGAGAAAGGCAGACAGAGAGACACTCACAGAGAGAGATTATCACAAGGATCTGGAAATACAGAAGGGAGCCAT
GCTAACAGAACACTGTTGAAGGTTCCCTCCCTCTGACCAGGTTATACCTGGATCTCAGGAGCTAACACTGGGATTTCAGGAGAAGCAAAGGTCT
GAACTTTCATGCACAGACAGTGTTTTACCACAGCAGCTCATCTTCAAATACGCTTAGCCTCCCTCACCCAGAGAGCCCTGCTTCCCTTCTCTGA
GAGTCCAGATAGGTAGTGCTCAGCAGCATAGCAGAGCCAAGAACTGAGTAATGGGCTGGAGAGCGACCTCGAACCACTCAGGAATGCATATCAT
CAGGTGTCAGGTATGGAGAGAAACAGAGCTGACTGTGGGGGTCAAAACAGAGAGACTTCTTCACAGTGTATTTTAAAGGCATTTTAAGCTAGGT
ATGATGGTGCAGGCCTGTAATCCGGACGCACAACAAAGGCAGGGCAGGAGGATGAGAAGTTCAAAGTCAACCTCACTATGTGTTTGAAGCCAGT
CTCATACTTAAGACCTAGTCTCAAAAATAAAGAAGAGAAAGAAAAGGAAAGAAAAGGAAAGGAAAGGAAAGGAAAGGAAAGGAAAGGAAAGNNN
NNNNNNNNNNNNNNNNNGAAAGGAAAGGAAAGGAAAGGAAAGGAAAGATACTGTAGCCAGGCACCTGCCTATCTTTCTCTGTAGCAGACATTCA
GAGATGTGCTTTTCTGAGGACAAACTGTCACAGTGAAACTTACTTCCATACATAACGAACTTCAGACCTTTGACCTCTTCCTAAAGCCCAACAC
TAGCTCAAAGGCTCTGACCCTTCAGACAAAGAACAGGATACTCTCTCTGGAAAACCCAGCTCTTCCAGCATGAACAGAGGGGTGCCTAGAACAG
GGATCACTTTACAGTGAGTGTGTTGGGAGTGACAACTGCCTCCCACCTGCAGGAATTTCCAGAAAGCAATGTGGGGTGCAGGGGTAGTGGTGCA
AGTCTTTAATCTTAGCACTCCAGAGGCAGACTAGTGGTTTTCTGGGTTCAAGGCCAGCCTGGCCTATCTAGAGAGTTCTAGGACAGCCAGGATC
TCTGAATTTGAGTCTAGCCTGGTCTACAGAATGAGTTCCAGGACAGCTGGGCACAAATTTAATAATGTAAATACATAATAACAACATTTCATCA
GGAGTTTTGAAGGGTATGTTCACGAGAGACAAGGGAGGACATGAGGAGAAAGGCAAAGACATAAGGCAGGAGCTTTCCTAGAACTGATGTTTTA
GACCACACAGGCCGGACTGCAAGGATGGGGATAGAGAGAGAGATGACTTGGGTCAAGAGCTCTTGATGTGCTTTTTAGAGGAACAGCATTTGGT
TCTCAGCATCTACACCAAGGGACTCATAACTGATTGTAACTCCAGCTCCTGCTACCTTCTGGCACATGCACACACATGGCAGCACCTTAACACA
CACACACACACACATAAAACATTTTTAATCTACAAAAATAGGGAAGCCATCTTCCCCAAGATGTATTATGAAATTCAGAGCACAACCTAAAGAA
ACCCTCCAACCTTCAAAGAGACCACTTGATGTCCCTGCAAAGGTTGAGGGTGGGAGGAGGAGAGCAACAAGAGCATCATTGGAAGCTAGAAGAA
ATTTGAGCAACACTTTGGGATTCCGAGTGATAAAACGATTTTAATCCAAGAATTTTATTCCCAGCCAGAGTATGAAAGATTTGCTTTTCTACTA
CATTAGAAAAGGTTTGACTTTAGACAGTATCATTTCCCAAGGTTTACCAGGTGGCTGTGAGTACGTAGGGGTGTGTGTGTGTGTGTGTGTGTGT
GTGTGTGTNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNGTTTCTCTGTATAGCCCTGGCTGTCCTGGAACTCACTCTGTAGACCAGGCTGGCCTCGAACTCATAAATCTGCCTGC
CTCTGCCTCCCAAGTGCTGGGATTAAAGGCGTGCGCCACCACCGCCCTGCAAAAGTTGCTTTTCTTATTTGACCATGGACTTCCTGTGATAGGA
CACGGGTAACTAGCTTAGTGGTAGATCTTTACCCACTGAGCCACCTTACCATCCCTTTAAAATTTTTTTTTCTTTTTTGAGACAAGGTTTCATG
TCTTCCAAGCTGACTTGAATTTTCAATGTAGCCATCTTCCAACCTCTTGCTTCCACCTCCCAAGTGCTGGGACTGCAGACATGTACTACCACTT
TTAGCTTATGAGATGCTGGGGATCCAGTCCAGGCCTCCATGCCCAGCACGCAGGGACCCCATCAACTGAGCTTCATCTCCAACCCTGGCGATTG
GACATCTTAAAGATTGGATTGCTTAGAGAGGAGAAAATACAGGGCTGACCCATAACGAGTGTGTTAATCACATAACAGAAAGTTGTATGGGAAA
GGAAATGTCACCTTAGAGTAAGCCTGCTAACACAGCCAGAACCGTGGGCAGTGAGTACTGAAGGGAGAGATCACGCTACTGTTTTCAGGGCGGG
AACGGGGAGGGTTTGGCTGTATAGTGGGAGGTAAGAGGGCGTAATAATTTTCACACTTCATGGTAAGAAAGCAACAGAAAATACCTCCCATTCA
AAAATCAAGACACAGCAAGACAAGTGCTGTGACAGCCCGCATACCACAAGGCACTTCCTTGCGTGGTCAGGAAACACACCACACAGGCAAGCAC
CGGCCCCATCCCTGTTTTAACAGCAGGTCTCAGTGGCTATTGAGATTCTTGACCAAGTAAAACAAAACCAAGAGAGACTGAAAAAGGAGAGATG
GCAAGAGTCTCCTCTCAGAAAGCCTGGCTGCAAGAGAGAGGGATGTGGGCGCTGGACTAGCTGGATAAGCCAGGCATGGTGTTATTCTTCCATG
CCCATGGGCCAGTTTGATAGGCAGGAAGGGTAAAGAAGCAGTGGAAAGTCAGGTGAGGTGCAAGCAGGGCCGAAGCCAGAATGATCAGAGCATT
AAGCCCATGCTGTCCAATATGAATCCTTGCAAGCCACAACCTGAGCTGCTTGGATAGTTACTAGCTCTCAGGTAGTTATAGTGAAAGGATAGCT
AGAGCTGGACGGTGGTGGCACTTGCCTTTCATCCCAGCACTCTGAGACAGAGGCAGGTGGATCTCAGTGAGTTAGAGGCCAGTGGTCTACAAAG
TTCTGAGACAGCCAGGGCTGTTACACAGGGAGACCCTGTCTTGAAAAACAAAACAACAAAAAAGAATACACTGAGAGTTTGGAGAGATGGCTCA
GTGGTTAAGAGCACTGACTGATCTTCCAGAGGTCCTGAGTTCAATTCCCAGCAACCACATGGTGGCTCACAACCATCTGTAATGGGATCTGATG
CCTACTTCTGGTGTGTCTGAAGTCAGCTACAGTGAACTCATATACATAGAATAAAAAAAATCTTTTTAAAAAAAGAATACACAGTTAAAATACA
CTTGGAGTCCTTTGAAAGGCAGTCTTACGGTGTAGCTCAGACTGGCCTCCAGCCCTCAGTACCCCAGCCTTAGCATCTCCAGTGCCACTGTGAT
TGTTCATAGTCATGCTCCTCCAGCACGCTGGGCTGGCTTTGATCTTTCATCACTCTCTCCTCCTTCTGTCCATTTTATGACAAATGACCTGTCA
CCATAGGGAAGGCAGTGGCCAGTGACACGTGGATATAACCCATGTCTCAGACTCATGGTAGTTTTACAGAGCAGGTGGGAAGCAGGCAAGGTCC
TTGAGAAGGAAGCTTCTAGCAACCTAATAGTTTCCTGCCTCGGTTTAAAATGTTTTAGCTGTCATGGTAATGGAACAGACTTACTGGGTAAAAT
GAGACCTTCAATATGTGAACATAATGCACTGTGGCCAAGCAGGCTAGGAAGCATCCTTATCGCCTGAAAAATCATTTCAACTAGGCATGATGAT
GCTTATCTGCCATTCCAGCACTTGGAGGCACAGACAGGAGAACTGCCACAAGTTCAAGGCTAGCCTGGGCTATGTCACTGAGTTCCAGACCATC
CAGGGGCTACATAGTGAGAACCTGTCTCAAAATGAATTTTTTTTTCTGTTTGAATGTTCAAAACCTACTCGTCATTTTGAAGCAGTACTTTGTC
TACTGTGGTGGACGCTTTGTGCTATAGAATACCAACAGATATCTATTCCTCCCTTCCAATGATGACCTCTTCAAGCTCCTACTACTTCTTAGCA
TTGCCACACTTGGGACCCAGCATCCAGCATGAGAACCTTGGCCACAGCCAGATCTAAACAATAGCTAACCCCATGGCCAGCTTGTTATTTCAGT
AGTGCAGTGGATGTTGAAATTATGAATGCAGTATCCTGCATGTGTTTCCTACTTTGGGGCTTAAAAGACTCAGTGTGTGCTTTGCACATAACGG
TGCTTGTTGGAATGTAAGAGGAAAGACACCAGGAGCTGTCCAGAGCAGATGCTTACAGCCTGGGCAGGAAGGCTACATGTCTGGAGCAGAGGAA
CTGGGATGGTCCCTGTGGTGGAGGGGGCTTGGAATCTGAGGGCAATGGACAAGGCCCCCTTGTCCCCACGCATTTCCCTACAGAAAAGGAGGTA
GGAGTCCCTACTTCTTGAAACACCTATTTCCTTCCCTGTAAAGGGAGCAAGGTCTACATTGTTCATAGGGAAAGCTGCTGTCCATCCCGGCCCT
GTGAGTATAGGGAGCACTCTAAGCAGCCCAGCTCAGTCTTCACACCGTAGCATCTGCTCTCCACATTGTCCCTCCAGCATCCCCAACCTGAAGC
TCCTTGCTTCCTGTACTCCGTCCTAGACACTCCTGCAAAGAGAATTAGCTAGACCCACAGGGAGAAAGCAGCTCCCAGGCTCCTGAGGGCTTGG
ATCTGGCAGAGAAGACCCAGGTCACCATAGAGAAGATAGTAGCACTAGGTGACCAAAGAGAGGAGTCTGGGGGCGGGGGCTTATGGAGAAGGTG
GGCTGCAGAGAGCAGACCCCCTGGTTGAGCCGCGCTAGGGACCAGTGCAGACACACTTCTGTGCTTGCCTCCTCAGGCCATCGCCAGTCACATG
CACCTCAAGTGCAAATGCCACGGGCTATCTGGCAGCTGTGAAGTGAAGACCTGCTGGTGGTCGCAGCCGGACTTCCGCACCATCGGGGATTTCC
TCAAGGACAAGTATGACAGCGCCTCGGAGATGGTGGTAGAGAAACACCGAGAGTCTCGTGGCTGGGTGGAGACCCTGAGGCCACGTTACACGTA
CTTCAAGGTGCCGACAGAGCGCGACCTGGTCTACTACGAGGCCTCACCCAACTTCTGCGAGCCTAACCCCGAAACCGGCTCCTTCGGGACGCGT
GACCGCACCTGCAATGTGAGCTCGCATGGCATAGATGGGTGCGACCTGTTGTGCTGCGGGCGCGGGCATAACGCGCGCACTGAGCGACGGAGGG
AGAAATGCCACTGTGTTTTCCATTGGTGCTGCTACGTCAGCTGCCAGGAGTGCACACGTGTCTATGACGTGCACACCTGCAAGTAGGAGAGCTC
CTAACACGGGAGCAGGGTTCATTCCGAGGGGCAAGGTTCCTACCTGGGGGCGGGGTTCCTACTTGGAGGGGTCTCTTACTTGGGGACTCGGTTC
TTACTAGAGGGCGGAGATCCTACCTGTGAGGGTCTCATACCTAAGGACCCGGTTTCTGCCTTCAGCCTGGGCTCCTATTTGGGATCTGGGTTCC
TTTTTAGGGGAGAAGCTCCTGTCTGGGATACGGGTTTCTGCCCGAGGGTGGGGCTCCACTTGGGGATGGAATTCCAATTTGGGCCGGAAGTCCT
ACCTCAATGGCTTGGACTCCTCTCTTGACCCGACAGGGCTCAAATGGAGACAGGTAAGCTACTCCCTCAACTAGGTGGGGTTCGTGTGGATGGG
TGGGAGGGGAGAGATTAGGGTCCCTCCTCCCAGAGGCACTGCTCTATCTAGATACATGAGAGGGTGCTTCAGGGTGGGCCCTATTTGGGCTTGA
GGATCCAGTGGGGGCGGGGCTTCACCCCGACTGGGTGGAACTTTTGGAGATCCCCTTCCACTGGGGCAAGGCTTCACTGAAGACTCATGGGATG
GAGCTCCACGGAAGGAGGAGTTCCTGAGCGAGCCTGGGCTCTGAGCAGGCCATCCAGCTCCCATCTGGCCCCTTTCCAGTCCTGGTGTAAGGTT
CAACCTGCAAGCCTCATCTGCACAGAGCAGGATCTCCTGGCAGAATGAGGCATGGAGAAGAACTCAGGGGTGATACCAAGACCTAACAAACCCC
GTGCCTGGGTACCTCTTTTAAAGCTCTGCACCCCTTCTGCAAGGGCTTTCCTAGTCTCCTTGGCAGAGCTTTCCTGAGGAAGATTTGCAGTCCC
CCAGAGTTCAAGTGAACACCCATAGAACAGAACAGACTCTATCCTGAGTAGAGAGGGTTCTCTAGGAACCTCTATGGGGGCTGCTAGGAAGGAT
CCTGGGCATGACACCCTCCTACGATAGCCTGCATCCGCTCTGACACTTAATACTCAGATCTCCCGGGAAACCCAGCTCATCCGGTCCGTGATGT
CCATGCCCCAAATGCCTCAGAGATGTTGCCTCACTTTGAGTTGTATGAACTTCGGAGACATGGGGACACAGTCAAGCCGCAGAGCCAGGGTTGT
TTCAGGACCCATCTGATTCCCCAGAGCCTGCTGTTGAGGCAATGGTCACCAGATCCGTTGGCCACCAGCCTGTCCCGAGCTTCTCTAGTGTCTG
TCTGGCCTGGAAGTGAGGTGCTACATACAGGTCATCTGCCACAAGAGCTTCCTGATTGCTACCACTGTGAACCGTCCCTCCCCCCTCCAGACAG
GGGAGGGGATGTGGCCATACAGGAGTGTGCCTGGAGAGCGCGGAAAGAGGAAGAGAGGCTGCAAATGCGTGGTGACTGACTGTCTTCTGCCTGG
AACTTTGCGTTCGCGCTTGTAACTTTATTTTCAATGCTGCTATATCCACCCACCACTGGATTTAGACAAAAGTGATTTTCTTTTTTTTTTCTTT
TCTTTCTATGAAAGAAATTATTTTAGTTTATAGTATGTTTGTTTCAAATAATGGGGAAAGTAAAAAGAGAGAAAAAAATAAGTTTGTGGTTTGT
TCTAGATTTTCCCTTTTATTAACAAGCATTAGGTGTGCATACTAATGGAGTTTGGTATGGAATTCTTCGATGATCCTCCCCACCCCCAAGGGTA
ATTCACACCTCCCTGTCCCTTATCACTTGTGGGTCTGCACTATGCTGAGAACCTAAACATGCCAACTCAGGTTCAGGGTCCCAGAGCCAAGTAG
AGACTGTCGACAAGCCTCCAGGGAGGGAGGTGGGGTCAGAATTCCAAGAAAAGAGAATTGAAATAGCTAAGTCAAGAAAAGAGCCGGTCAACTG
TAGAAAAATCTGGAACAGGGTAGATCTGAGAGCTTGAATAGAGGGGTGCAGGTGGGTTAAGTTTAAGCAAGTATGGGAGCGGGGTAAGCATACA
GTAGTTAGGATACTTTGGTAGTTCCAGTGAACAGATGAAAAGTCACAGGAGAAGAGGCTTAACCAAGACCCCCTCACACGCTGGGGATGCAGAG
GAGACGTGGAGCAGATCGCCTCCCTGTGACTTCATGTTCTCACAGCCCATTCCCCAGAATCATGTATTTAGGGTGGACAGCCCTTGGTATTTGA
GGACAGTGGCCTGGCTTGCTGCTGCTGGCTTCTTATGTGTAAAGTCAACAGATCTTAGACTTTAGTCCTCAGACCCTGTCACCAGGAATGTTTC
AGATTCTCACCCAAAGAAAGCAATGGGAGGGAGGGGTCTTTGCCTCAGGAGCTGCCATGTTCCCACTTGCTTTCACAGGGGCGCCAGGGCCTCA
GTCAAGACAGGCAAGGCTAATCATCCTGTTGGCAAACCAAATGATGGGGCAGGTGTCAGGGCGCCCTGGGAGTGAGGCTAGAGATCTCTCACAA
AGCCATACTTCTTTTAAGGCCACACATCTGCCCAAGCAAGCCTACCATGTCTCTGCTCCTCTCCCTGGTCCCCCATCTCCATCCCCATACTGTC
CCCACCTCTGAGTCCTATTCTGATGTCCAGATGTCAGGGCAAAGGATGCCCTGCAAAGCTGAGAGCAGCAGTCCCAGTCCTACCTCTCAAAACC
CAAGCTGAAGCCGAGTATAGCAGTGCACGCCTTTAATCCCAGCACTCGGGAGGCAGAGGCAGGTGGATCTCTGAGTTACAGGACAGCCTGGTCT
ACAGAGTGAGTTCCAGGACAGCCAGGGCTACACAGAGAAACCTTGTCTTGAAAAACCCCAAGAAATAAAAAAATTAAAATTAAAATTAAAAAAA
AACAGGGGGAAAAAAGCCCAAGTTGAGCACAGTCATTGTTCTTGTGTCCCATTTATATCCCAACCCAGATCCAAGTGATTTGAAAGCCAGTTAG
AAGATGGCCATAAGAATAAGCCTAGGAAGTGTCTTATGATGTCCAAGGTCCTTTAGCTGTCGTGATACATCATGACCTCATGAACCTTAGAACT
AGGAAACCCTGCTGGCCTCTAGAGGACCAAGGGAAAGGATCTGAAATATAGTCACTGTTGACATAGGGGAATGCTGGTTAGGACAACCAGGAAG
ATCATTAAGTGTCCTCTCAACCCTCCCAGGGGACAGTGGGACTGCCTGTCCCAGAAGAGGGGAAGATAATTCCCAGACCCTAAGAGCTGGGAAG
AGAGGCAGTGAAGTGAGAGGAGAAACAGGCAAGGCCCTTCTAGACCCATTTGATTCAACACTGTAGAGTTGGTCGGTGATGACAGCTAAAGCCG
AGGCTGCTGTCCACATGCCCTGTGAATTTGTCCACAGGCCTGGTAACAGAATACTATGTCACACTGCCTGAAACTCTGAATGCCACTTGTCAGA
GCAGGCTGCCACTGGTTTTATGGCAGGACCACTTAAGGGGGTCCTTAAACCTTCAAAGCAATCACAAGCAGGCCTAAGACCATACTAGACTCAC
CTGTATCCTTAAAGAAGAATATTGAGGAGCCTCCAGGTCTTGGGACATAAAGATGAGACCAGGCATGGCTCCACTGCATGCTCCAGGCACATCT
TTTGTTCACCAACCCACTGGCTAAGCTTTCTGATCTTCCAGAACACAGATTCAACCTGTAGCTGCTGATCCATGGTCTGAGTCAGCTCAGTCTA
CCACACTAAGATTCTAGACCCTCCGTGGTCACACTGTCATACCTAAACACCAATGTGCCCAGAGTCCAAGTAGGGTACAAAAGGAATAGCCAGC
CTGGCTTGAAGGTATTGGCCTATAATCCAAGCTGCCCCAGAAGCTAAGGCAGAAGGACCACAAACTCATGATTGCCTGGGATACAGAACAAGGT
GAAGATCAGCTAAAGCGATTGAGTGAGACCCTGTCACCAAATGAAAAGTGATAAAGAACTGGGGTGTACCTCAGTGACAGAGGACTTTCCTAAT
GTGTGAGGCTCTGGACTCCACCAAAGCACTGAAAAGAAAAGAAAAAAAAAAATCACAGACTGTGAGGGATGCTGACAGCTAAGTGGCCTTGGGA
AAGGAGTGCCATCCTGGGGCTCTCTGGCCTCTGGCCAGGTCTGTTTGGGGTTTGTGGAGGAAGAGGCCCAGGTTCTGGATTCTCAGTACCTCTT
CTTTAGCCTTCTGATTCCCTGACTCCTGGCAGGCAGGCAGGCACTCCAATGACATCTATGCCTTCACACGGCTCCCTCTTCTCATTCTCAGGGC
TGCAGGTGTATGTACACCAGGCCCTGACCTACTATTGGCAGGACTACCTAGGATTTCCCAGTAAGGGCAAGGGTCAGTGGGGGGGTAGGGAGGG
CAACTAGCTGCAGGTGGACACTCCAAAAAGAGGATGCCATGGTCCAGGGCCAGCCAAGGCAGAAGGATGGGCGCCCATTCTCCCCTCAGGGCAG
TGAATCATCAGGTGAGTGGGCAGGTTCAGGAAGAGTCCAATTAGCCCGCTGGGCCATCTTGGGTGCTAACAATAATAGAGCCATCAGTCCCTAG
GGCAGTGATGAGAGTCCTCAGTTAAGCCTGTCCGACAGATGAGTAGTCCCTCTAAGTTGGCACCCTGGGCAGGAGCCACCCTAATTGCTACCTG
CTGCCCTAGTGCCCAGATGAAAGGGACAGACTCGACTGGGAGGCGGCAGGGCAGTGGCAAAACCAGCATGTCACGAGGGCCTGGAAAGTCAGAG
GCCAATGGGTATGGGGTGCCAGGCTGTCTCAAGACCCCAGTAGGGTCTAATGTCACATGGCTTTTGGGGATGCAGAGCCCATATGCCCAGGCAG
AAGACAGATCACTTTAGAAGGTCAAAGGCTAGGAAGCATATAGCCTACAGAGGAGGCAGCATTGGCCTGAGGGCTTGTCAGGGACACAGGTGGG
CTCTGGTGTCTGGTGTCAGTTTCTGAGCAGCAGCCCCTACAAGTCCAGGATGAAAGACTACCCTAGAGATTGTCTTGGCTATCCACAGGAGATA
CACGACTANNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNACCCCCCCAGTCCGAGGAGTGGGAAATCCCAGGCCCCACTCAGACCCAAGCTCGCAGCTGTGGGCCTCAGAAGCAGC
CTCTCTGGTGAGTTTACAGCCCCTCCCGCTGCCCTGGAGAAGGAGGAAATGAGCGGGAGGCCTGTTCACAGGTAGTTGAGACAGCCCCCGAATA
ACCATATAAAAGGGCCGGAGGCTGGGGGCCCCACCCAGCCAGGACAGGCTTGGCCATCCCCTTGCAAATCTCACACTCAGTAGCTTTGAAGTAC
TGGAAAGGGTGTGGCCAACCCCCACCTTAGGGCCCCACTGACCCTCCCTTTTTCTGTCACCCCCTCCCCTGGATGCCCACTTTCTGGGTCCTGG
GGCTCAGTTTCCTCTCTTCAGAGACAGACTGGAGGCTGTCACCATGGCCTGGCCTCCAGGAAAACCTACTCTTGGTCAGCTGGGGTGCAGCCAG
CTGTGGTTCCCACCCCTGGCTTGGGACTGAGAAAATGGACCTGAGGACTAGCTTTCCATGATGTGGGCAAGTGTATGACCAGAAGCCAGAGTCC
CCAGCCCAAGCAGTCTTGGTAGTAGGATCTAGCACTGCCTTTTCCAGGGAACACTGGGGACAACAGGGCAGCCAGGAGACAATTTTGAGAAGTC
CCTAGGACCTTCCTGAAAGCTGACCACCCACTGTTGGGGTGAGAGAAAACTATGATGTTCTGTTGCTGTGTAGCTGGTGATCAATAGCTGGGCC
AACTCTGAGTCCTAGCAAACCTGGCCCAGCCATAATGATCTACCTTGAAGGGACTTTGGGATACATTACTGCCCTGGAGTCCAGCGAGCGTCCT
GGGACAGATTCTTCTCCCCCTGGCTTTACCCTGCACCATGCCACAGGCCCCAGGTCCCACCTCTCTTCACCAGTCGCTCACCTTCCACAGCCTA
GTCCTTTCAGGGGTTCCCAAATTTCTTTTCAGAAACTCTCTTGGAGGGGGGGGTACCCAGAACAACACACTCTGAGATTATAGCCTTTTAGGAT
CATCATCCTCTCTAAGGACCTCTGACTTCCAGAGCCAGAGTAGAAACCAGGTATACACACTTACTATCCCTACAGCCCTAAGGGGCCAGCGTGC
CATCCTACTGGAGTCTAGTATTGGACAGATATATGAGTCTGGGATAGTCCAGTTGGCTAGAGTCTTTCATAGATTTGAATTGAACTGTACTCTC
AATGCTACTCTTGGGCTATGTGGGAAGGCTTCCAACAAGGTGCTGCTTCTGCCTTCTGATGTTTGTTAATTCTGAGATACCATACAGATGCCAA
GACAAATAGTGCAGGGCAGCTAGGAAGCCCTGGAAAGGGCCCCCAACTCCAGGAGCTTGGGCACTGCCTACCCTTCCTACCCTGCCCTGCTTTG
CCTGGTCGGGCAGCTGGGACGGAGCTGCAAGCTGTCAGAAGGTGCTAATTGTTGTTTAGCTCTAAGCCGGCTCCTTGCTCTGGTCTTTTTTCCA
CCCTCTCTCTCTTTTTTATTTGAGCGTCTAGAACTCATGGTAAATAGTCATTTGGCTCTCAGAGCCCTGCAAATTGGTGTTTATGGTGTGAAGA
TGAGGAGGACCCAGGACTAGTCCACCAAAACACAGGCCAGTGAAGAGGGAGGAGAGAGGGAAGGAGGCATAGGCCCAGCTGGGACTGGGCATCC
TCCACGTCCCATCCAGGCCTAGCTCTCTGCTGCTTGATCCAGGAGAGCTTCCAGGAAGAGGCTCCTCCGTCATCCTTATCAGTGGGGAGACTGG
AGATTGAAGAACTAGAGCAGGACCCATATTTAGCATAGGAGATANNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNGAAGGAAGGAAGGAAGGAAAGGGAGGTGA
ATGGGGAGGGAGGGAGGGAGAGAAAGAGAGAGACAGAAAAATCCAAGTACATGGGCCCTTCTTACCCAGTGGCCTCCCAGATTTGAGACAGGAA
TGTATGGCTTCTGTATGATGCTTGTGGGGTGCAGGGTGTGGAAGGCAGGTGCCAGAGGAATTCCAAGCCCGAGCAGATGGCTGGGAGGGCTTCT
CAGGAGAAGGGGACTTCCCCAGTTGTTCGCCTCTCAGTGCAGAGAGCAGGCAGGCCTGTGCAGAACCCTCAGACTCCTTGGCAGTGGTGGCTCA
GCAGCCTCCCTTCCCCATCCCCCAGGCTCCTCAGCTCCCTCTACTGCAGGAAGCCCAGGGTGTTCCCACAGCCTCACTCTGTCCCTTCTCCATT
CCTGGCACAAGCTTGAGACACTCTGCCACCCCACAGTGCCAGACTTTATAAAGCAAGCTGGCTTCTAAGCAGCCCACCAGACACACAGCCCTGG
AGGGGCGAGTCCCAAAGCTCTAAGAAGGCCATTTGACGTCTGTAGCTTGGAGTAGACTCCAGGGATTACACATGCTTCTTCCCCGACTGTTTTT
ATTGTCCAGGTCTGGGTTGGGGCAATAAAGTGAAGTGCCAGCTTGCACCTCGGGGACCAGGGACTAATCTGGTGGGAGTCGTGACCCAGGCAAC
AGTCCATATGCCATGGACCTGATCCTACACCTCCTCTGCTTAAGGATATTCCATGGTTTCCCTGTGTCCACCAGGTGAAGTTTAACTTCTCAAG
CCTACGTACCTGGCAATCTATCCCCTTAAAAGAGTCCTGATGTATGGCGTGAGAGACTGAGAAACTGTCCTGCACAGGAGGTGTATACGGACAT
TACTCTTCAGACTGCAACGGACGCCCGCCTCTATCACTCATTCCCTCAGATGCTCTCGGACCTCCATGCCAGCCTTCTCACCAGTTCTAAAAGA
AGTCTCTCTCGGGTCTCTAGCCTTCTCTCTCCCAGGTCCTGAACTTTCAATGGGTTCTTCTGACTCTGGAACTGGTTGCAATTCCTTGACCTTC
TCTGGTCACATTCAGGAGCCCCCATCACTGTCTCTGGGGGGACACTGGAAGTACCAGCTTGACCTCAGGGACCTGTATGGCAATAAAGGGGGGG
AGGGGATTCTGATGTCAGTGTAACCTTGATCATTCCTGGGGCTTGGAAGCATACCCAGCTGAACCAAGAGCTTGAGCTTCCAAGTGACCCATCA
ACCAGAGGACCCCACACCTGTGAGAGCAGGGAGGGCGGCCTCAGAACCGAGGCCTTGTCTGGTCCTTGCTGTTGTATGATGGGAGGTTGTTTGA
CAGATGCAATGGATGAACCATGCAGAATCCAAGCATGACAGGGATGAGGCCACAGCGCAGGTGGCGAAGGGCCATGTGAGGTCTGGGAAGGTGG
AAAGGATGCAGGAGAGGACCTTACAGGGGAGGAGGATGCCAGTGGAGATGGTTCAGGAGAGGACCTCGTGGGCGGGCCTGAGGAGACAGTGCAG
ATGAGGTGTCAGTAAGGAGGATGCAAGCAAGAAAGATGCAGGAGATGATGGAGAAGCTGAAGAAGGCACTGAAGAAGGCACAGGGAAGAAGAGT
GCATGTTAGGATGTTCAAGCAGAGACGGTGCAGTGGTGAGGGTGAAGGTGGAGGAGATTCAGATGAGGATGGTACAGGTGGGATCCTGCATATG
CTATCAGGCAGGTGGCCCAGAACAATGAGCCTGGCCATGGTCTATGAGGGACGCACACACTGGGAATGGTACCTACAAGGTTCTGAAAGTGCAG
GCTTTTGGAGACCACCCTCCCCCCAAAGAAAGGGGTTTCTACCCCTTCTCCAGTTCTTCCCTTGTGCTTTAACCAAAAGCAAGTCTTGTGCACT
GTGCCCAACCAACCCTGCACTCTGCTGAACCTGAGGCAAGAGTAGAAAGGCTGCTGTCTTTTCTAACCTGTACCCTGAGCCAACTAGGTCAGAG
TCAGAGGCCTCACATACGTGAGTAGCCCTGGTTGGAGGGATCTCTAAACCCCACCACAGTCCTGGGACAGCCTGCTGTTTCCTGGGGTCAGGGG
TCTTCCATGGGCCCACTGCCGTCCTAGTCAGCTTTGCTTCTGTTACTTGGGGATTTTTTCCAGTCTTTCCCTTCCGTTTAGAAATCCTTTTCAG
GACAGATCCCTGACAGAGGTGCTGACATCATTAGGCCTCCTCAAGGGCACAGGCCCACCCTTCTGCTCTGGCTACCCCTATCCCTAACCCACAG
CCCAGCTCTGCCAGGGTCCAGGCCTGGCTGGCAGCCCGCAGACTACCCAGACCAATGTGTTGGCTCTAGGCAGGCAGGCATCCTGAAGGGTCTA
ACAGTGTCCTTTGCCAACTCTCCACTGAAGCCGATGCTGGCCTGTGCCTTTCCCTCTGCCTCTTATCTTCTTTCCCTCGCTGCCACCACACTCA
GGAATCATGGGACCCTCTGGTCTTTGAGCTACAGCTCTGTGCAGGTAGCACAGCCTCCTCCTTCCCTTCCCTCCCTCATACATGTGGCTGTCGA
CCCCTGTCTCTGCAATAAGCTCTGGGTGCCTCTGACTACGCCTCTGACAGTCCTAACTAGTGTGGGCTGGAACACAGTGGCCAACATATGTAAA
TGCTGGAAACCCCTCTCATAATAGAGATGGCAGGTGTGGAGCTCCGTTGAGCCTGAAGGTAGAGGGCAGAGTAGTGATGGTCTACGGGCCACAT
CTGGTCCCTAATGGTGGGAGTCAGGACCTGCCTGGTTTCCTCAGAGCCAGAGTGGGAATACAGAGGCATCTGGTATCTTAGGTCAAGGGATTTC
TCCAGCCCTTACAATGTGAGCAGGATGTTGCTGTCAAGGCTACTGGACAGCTCCAGGCTCTGGAGACCACCTGAGGAGATATGGACGGGCCAGC
CCCTGGCACAGTCCAGATTTTACCACGGACCCAGCCTGGGGCACAGGTGTTAGGGGTGGGGCTCATGTTGGCGGGAGTGCTGGCTGCGGATGCT
TCAGCTGGGCTTTTTAATTAACCTCTGCTGGCTGCTCCAAAGGGCCCCAATTAAGTGCAGAATGCGATGGCAGAGTGGGGTCAAAGAGAGGAGC
TGGTTTTAATTAAAGTGCAGTGGCCTGGGCAGGAGCGCCCCCGCCTGACCGCCTCCTCAACAGGCCCTGCTCAGCTCAAAGGCGGTCACATGAC
GGGTAGATGACAGAGAACTCTGCAGTCCTGTCTGGCTGAGATCCCACCAGATAACAGCAGGACCAAGGGCTGCCTTGTCTAGCCTGTGTGTCCA
CTGCCAGGCAGCCCCTCTTCGAGCCACTTTGTCCTCTCTGACCTCTTCTCATTTGTCACCTGACCTGGAATAGCCATCTGCTACCCTCTCAATA
AGCAGGTCCCTGGTATAATAAGCATCTGTGGTATGTCTACTTCAGAAGCAGCAAAAGGAATAAAACAAACAATTCCATCAGATGTGCATGGGCA
ATGGGATTAAGCAAAATGGGGGGGGGGCGGGGGAGGGGGAAGGCCAGTGGAGAGCTGTTTAACCTGGTAAAGGAGCAGATAGCAGCAGGAAGGG
GAGGGAGCTCACCTGGAGAGGTAAGGGCCTGGTAAAAGCCAGAGCAGGATCTGGCCAGGGCTGAGTTGCTGAGGTACCAGCAAGGCCAAAGCCT
GCAAGCAAAAATAAGAGAGAAAGCCAGGCTTTTCTCCAAATGGCCAGAAGACTACTGAGGACTCTCAACATAATTAGCTTTCCAGGAAATGTAA
TGAAAAACCCTGGGCGAGGGCCGGTGAGCTACCACCAAGCCCGGAACCCACACAGTAAAAGACACAGCTGACTTCTGCACACACACAAATGCTT
GTATAAGCATATACTTATACACATGCATGCATGCACTTGGAAGTGGAGGCAGCAGCATTAGAAGTTCAAGGTTACCCTCAGTTACATAGGGATT
CTGGGCCTGCCTGAGCTACATGAGGTCTTGTTTCAAAACAACATCAGCAACAACAAAATTGGATCCTAAGGTGACAGTAGAGTGCCAGCCTCCC
TCCTGCTGCAAATTCCAGGCAGGTGTTCCCCACCTATTACAATGGTCCCAAGTAATTTTTTTTAATGTTATGGGCATTTTAACACACACACACA
CACACACACACACACACACACACACACACACACACACAGAGAGAGAGAGAGAGAGAGAGCATGCGTGCAAGGGGATAAGGCCTTTGTGCTTCAC
TTGGGAAGAACAGGTGTTTCATGAGTTTCTTGTGAGGAGAAACATTTGCTAGGCATCATAGCCCTCTAGGAATCTCACTGGCTAGGAAGACAGG
ATGTCTTCCAGCATCTTGACCCA
MOUSE SEQUENCE—mRNA
GAATTCATGTCTTACGGTCAAGGCAGAGGGCCCAGCGCCACTGCAGCCGCGCCACCTCCCAGGGCCGGGCCAGCCCAGGCGTCCGCGCTCTCGG
GGTGGACTCCCCCCGCTGCGCGCTCAAGCCGGCGATGGCTCCTCTCGGATACCTCTTAGTGCTCTGCAGCCTGAAGCAGGCTCTGGGCAGCTAC
CCGATCTGGTGGTCCTTGGCTGTGGGACCCCAGTACTCCTCTCTGAGCACTCAGCCCATTCTCTGTGCCAGCATCCCAGGCCTGGTACCGAAGC
AGCTGCGCTTCTGCAGGAACTACGTGGAGATCATGCCCAGCGTGGCTGAGGGTGTCAAAGCGGGCATCCAGGAGTGCCAGCACCAGTTCCGAGG
CCGGCGTTGGAACTGCACCACCGTCAGCAACAGCCTGGCCATCTTTGGCCCTGTTCTGGACAAAGCCACCCGGGAGTCAGCCTTTGTCCATGCC
ATCGCCTCCGCTGGAGTAGCTTTCGCAGTGACACGCTCCTGTGCAGAGGGATCAGCTGCTATCTGTGGGTGCAGCAGCCGCCTCCAGGGCTCCC
CAGGCGAGGGCTGGAAGTGGGGCGGCTGTAGTGAGGACATTGAATTTGGAGGAATGGTCTCTCGGGAGTTTGCCGATGCCAGGGAGAACCGGCC
GGATGCCCGCTCTGCCATGAACCGTCACAACAATGAGGCTGGGCGCCAGGCCATCGCCAGTCACATGCACCTCAAGTGCAAATGCCACGGGCTA
TCTGGCAGCTGTGAAGTGAAGACCTGCTGGTGGTCGCAGCCGGACTTCCGCACCATCGGGGATTTCCTCAAGGACAAGTATGACAGTGCCTCGG
AGATGGTGGTAGAGAAACACCGAGAGTCTCGTGGCTGGGTGGAGACCCTGAGGCCACGTTACACGTACTTCAAGGTGCCGACAGAACGCGACCT
GGTCTACTACGAGGCCTCACCCAACTTCTGCGAACCTAACCCCGAAACCGGCTCCTTCGGGACGCGTGACCGCACCTGCAATGTGAGCTCGCAT
GGCATAGATGGGTGCGACCTGTTGTGCTGCGGGCGCGGGCATAACGCGCGCACTGAGCGACGGAGGGAGAAATGCCACTGTGTTTTCCATTGGT
GCTGCTACGTCAGCTGCCAGGAGTGCACACGTGTCTATGACGTGCACACCTGCAAGTAGGAGAGCTCCTAACACGGGAGCAGGGTTCATTCCGA
GGGGCAAGGTTCCTACCTGGGGGCGGGGTTCCTACTTGGAGGGGTCTCTTACTTGGGGACTCGGTTCTTACTTGAGGGCGGAGATCCTACCTGT
GAGGGTCTCATACCTAAGGACCCGGTTTCTGCCTTCAGCCTGGGCTCCTATTTGGGATCTGGGTTCCTTTTTAGGGGAGAAGCTCCTGTCTGGG
ATACGGGTTTCTGCCCGAGGGTGGGGCTCCACTTGGGGATGGAATTCCAATTTGGGCCGGAAGTCCTACCTCAATGGCTTGGACTCCTCTCTTG
ACCCGACAGGGCTCAAATGGAGACAGGTAAGCTACTCCCTCAACTAGGTGGGGTTCGTGCGGATGGGTGGGAGGGGAGAGATTAGGGTCCCTCC
TCCCAGAGGCACTGCTCTATCTAGATACATGAGAGGGTGCTTCAGGGTGGGCCCTATTTGGGCTTGAGGATCCCGTGGGGGCGGGGCTTCACCC
CGACTGGGTGGAACTTTTGGAGACCCCCTTCCACTGGGGCAAGGCTTCACTGAAGACTCATGGGATGGAGCTCCACGGAAGGAGGAGTTCCTGA
GCGAGCCTGGGCTCTGAGCAGGCCATCCAGCTCCCATCTGGCCCCTTTCCAGTCCTGGTGTAAGGTTCAACCTGCAAGCCTCATCTGCGCAGAG
CAGGATCTCCTGGCAGAATGAGGCATGGAGAAGAACTCAGGGGTGATACCAAGACCTAACAAACCCCGTGCCTGGGTACCTCTTTTAAAGCTCT
GCACCCCTTCTTCAAGGGCTTTCCTAGTCTCCTTGGCAGAGCTTTCCTGAGGAAGATTTGCAGTCCCCCAGAGTTCAAGTGAACACCCATAGAA
CAGAACAGACTCTATCCTGAGTAGAGAGGGTTCTCTAGGAATCTCTATGGGGACTGCTAGGAAGGATCCTGGGCATGACAGCCTCGTATGATAG
CCTGCATCCGCTCTGACACTTAATACTCAGATCTCCCGGGAAACCCAGCTCATCCGGTCCGTGATGTCCATGCCCCAAATGCCTCAGAGATGTT
GCCTCACTTTGAGTTGTATGAACTTCGGAGACATGGGGACACAGTCAAGCCGCAGAGCCAGGGTTGTTTCAGGACCCATCTGATTCCCCAGAGC
CTGCTGTTGAGGCAATGGTCACCAGATCCGTTGGCCACCACCCTGTCCCGAGCTTCTCTAGTGTCTGTCTGGCCTGGAAGTGAGGTGCTACATA
CAGCCCATCTGCCACAAGAGCTTCCTGATTGGTACCACTGTGAACCGTCCCTCCCCCTCCAGACAGGGGAGGGGATGTGGCCATACAGGAGTGT
GCCCGGAGAGCGCGGAAAGAGGAAGAGAGGCTGCACACGCGTGGTGACTGACTGTCTTCTGCCTGGAACTTTGCGTTCGCGCTTGTAACTTTAT
TTTCAATGCTGCTATATCCACCCACCACTGGATTTAGACAAAAGTGATTTTCTTTTTTTTTTTTTCTTTTCTTTCTATGAAAGAAATTATTTTA
GTTTATAGTATGTTTGTTTCAAATAATGGGGAAAGTAAAAAGAGAGAAAAAAAAAAAAAAAAAAAAAAAAAAAAAAAAAAAAAAAAAA
MOUSE SEQUENCE—CODING
ATGGCTCCTCTCGGATACCTCTTAGTGCTCTGCAGCCTGAAGCAGGCTCTGGGCAGCTACCCGATCTGGTGGTCCTTGGCTGTGGGACCCCAGT
ACTCCTCTCTGAGCACTCAGCCCATTCTCTGTGCCAGCATCCCAGGCCTGGTACCGAAGCAGCTGCGCTTCTGCAGGAACTACGTGGAGATCAT
GCCCAGCGTGGCTGAGGGTGTCAAAGCGGGCATCCAGGAGTGCCAGCACCAGTTCCGAGGCCGGCGTTGGAACTGCACCACCGTCAGCAACAGC
CTGGCCATCTTTGGCCCTGTTCTGGACAAAGCCACCCGGGAGTCAGCCTTTGTCCATGCCATCGCCTCCGCTGGAGTAGCTTTCGCAGTGACAC
GCTCCTGTGCAGAGGGATCAGCTGCTATCTGTGGGTGCAGCAGCCGCCTCCAGGGCTCCCCAGGCGAGGGCTGGAAGTGGGGCGGCTGTAGTGA
GGACATTGAATTTGGAGGAATGGTCTCTCGGGAGTTTGCCGATGCCAGGGAGAACCGGCCGGATGCCCGCTCTGCCATGAACCGTCACAACAAT
GAGGCTGGGCGCCAGGCCATCGCCAGTCACATGCACCTCAAGTGCAAATGCCACGGGCTATCTGGCAGCTGTGAAGTGAAGACCTGCTGGTGGT
CGCAGCCGGACTTCCGCACCATCGGGGATTTCCTCAAGGACAAGTATGACAGTGCCTCGGAGATGGTGGTAGAGAAACACCGAGAGTCTCGTGG
CTGGGTGGAGACCCTGAGGCCACGTTACACGTACTTCAAGGTGCCGACAGAACGCGACCTGGTCTACTACGAGGCCTCACCCAACTTCTGCGAA
CCTAACCCCGAAACCGGCTCCTTCGGGACGCGTGACCGCACCTGCAATGTGAGCTCGCATGGCATAGATGGGTGCGACCTGTTGTGCTGCGGGC
GCGGGCATAACGCGCGCACTGAGCGACGGAGGGAGAAATGCCACTGTGTTTTCCATTGGTGCTGCTACGTCAGCTGCCAGGAGTGCACACGTGT
CTATGACGTGCACACCTGCAAGTAG
HUMAN SEQUENCE—GENOMIC
CTATGGGAAAACTTCAAGCAGCCCAACATAGATGTAAGTGGAGTCCCTGAAGGCAGGACAGAGTTATCACAAGGAAGTGCCTTATGCATTTGTA
GAGGCTGAACATGTCCAAAATTCATAGGCCAGGAACTCAGGAAGACCATAGACAGGCTCAACCCATGGGCTTTCTGTCTCTATCCAGACAACCC
AGGAAAAAGAGAGAGGACAATGACTAATCTCAATAATTAGAAAGGTGACATCTCTACAGATTCTATAGATATTAAAAAATAAACAGGAATAAAC
ATCTACATGTGTATAAATTCAACTTCTGTAAAATGAACAAATTCCTTGAAAGACATCAACTACCAAAGCTCGCACAAGAAATAAATAAACAAAA
TGGTTCTATAGCTATTTAAAGAACTGAATTGTGGTTACAAGCCTTCCTACAAAGAAACTTAAAGGCCAGATGGCTTCATTGGTGAATTCTACCA
AACATTTAAGCAAGACATAATAACAATTTCCACAAACTCTTCCAGAAAATTAAAGAGTTGGAAATACTCATGAGTATCTCATGAGGCCAGAATT
ACCCTAATGCCCAAATCAGACAAAGACATTATAAGGAAAAAAAATTCTAGATCAGTACTTCTCAGGAACATAGATGCAAAAGTTCTTAACCAAG
TTTTAGCATATCCAATCCAACAATATATAAAGAGGATGATACACCATGAGCAAGTGGGATTTATCCCAGGAATGCAAGGTTAGTTTAATATTTG
AATATAAATTAATGCAATTCACTATATTAATTGGCTAAATAAAAACATATAATTGTCTCAATAGATGCAGAAAAATCATTTAATAAATTCCAAT
ATTTATATAAAAACTCTCAGCAGATTAGAACTAGCTGTGAACTGCCTAAACCTGATAAGTGGCATCTATGAAAAACTTCAGCTCACATCACACT
TTAATAAAACACTGTGTGTTTCCTCCCTAAACATCAAGAACAACACAAGCACATCCACTCTCACCACTTTTATTTGACACTGTCCTGGAGTTTC
TAGCTAGTGCAATAAGACAAGAAAAAGAAATGAAAGGCATCTAGAATTTAAAGGAAGAGGTAACCTGTCTTTATTCACAGGTAACACAATTGTT
TATATGGAAAACGCCAACAAATCTACAAAAAAGAATAAGTCTGCTGGAACTAATAAGTGAACTTAGGACAGTTGCAGGAAACAACATCAATGTA
CTAAAAGTCAGCACTTGCAACAAAACATGGAAATGGAAAGAAATAATACCACTTTAAATAGCTGATAGCTTCTGGACTAAGAGGAAGCTGCAGG
TAGAGGGGGCAGGTGGAAGATTTAGGAGAGTATGGAGGTGGTTGCTGAGCCTGATATGAGAAGAACCTGGACAGAACAGGAACAAATGCTTTGC
CGAAGCTTCTGGACCCACTGGGCTGGAGGACAACCAGCAAGGAACAGCAAGGAAGGAACCAGCAAGGAAGCTTCTGGACCCGTTGGGTTGGAGG
ACAACCTCAGCAACCCTCTGAGGAGGGAGAGACAGAAACTCTCTGCAGAGACTGCAGGTTGTGACACAGAAGCTGATGGCCTCGATTTCCCTCC
ATGAAATGGGACAGTAGGGTGCCGTTGACAGTCAGAAAGGGTGTGCAGGAGGTGGATGCTAGGGAGGAAGCCCACAAAGGGGCTGATGGACTGG
CCTTGTAAGAAACAAAACATCATGTCTCCTTGGAGCCATGTCTCCTGTGCCTCGCCAGGGACAGTGGGCCCCAGCTTCTTCGACCAGGGAAGAT
GGCAGTCAATGGATACCCCAATGGCTTTTTCTCTTCACCTGTTCCCCCCACTCTCCTGTTCCCTAGAACCATGTTCCCACATAAACCACCTGCA
CTCACTACTTGTATCATGTTCTAAAAATCCCACCATGTCCCTGGAGCTCAGGATCTTCAAGGCCAAGGGGAGGCCCTAAAGGTGAAGTTGGGGG
TGAACCAGAGTGAAGGAGAGCATCTCCAGTGATGAGGGAGTCCAAGACCCATGATCCTGGGTGATGGGGGTGGACAGGAGGAGCGTGACTGTCA
AGCTGCCCAGGCCCAGGCCCATGTGTGAACAGCAGCCCTGTGAATCTTGCTCTTGGCCTCTGAGCCTTGGCTCTGGCTTATGCTCCAAAGATGG
TGACACTGCATCTCCTGTGGTCTCAGGGATCAGCTGGGTCATGCAGGTTTCAGCAGGGGTTGTGGTCACCACTATTCCCATGAGAGAGGTCATA
CCCACAATGTAAAATCCCACTTGGGCTGCAGGACAGCCAAGGGCTAGGGAGGAGGCGCCGCTCATGTCACCCACAACCTTGCCCAGAAGGGGCA
CTGCCTCTAGGGGAAGATTGGACCCAGGCACCCCTTCAGTGCCGCCCTCCTCTGGCATCTCCACTTGGTGGCAGTATTGGCCCAGAAGCGGAAC
TCCAGCAAGCAGCCACCAACCGGTCCCTAAGAGCTGCTGTGGCAGCAAAACTTAGCTCTTTCCCAACAGGAAAGTCTGGGGAGTCTATTGCCCT
CATTTCACAGGGAAGTAAGTTATGGCCTGGGAACCAGGATTCTGGATAATTAATCTGCAGGATTCCAGAACTCCATGATACTGTGATTTTTTTT
TTTTTTTTTTTTGAGTAGTGGTCTCTCTCTGTCACCCAAGCTGGAGTGCAGTGGCACAGTCATGGCTCACTGCAGCCTCGAACTTGTGGCCTCA
ACCAGTCCTCTCACCTCAGCCTCCTGAATAGCTGGGACTACAGGTGCATACCACCATACTCAGCTAATTTTTTATAGAGACGGAGTCTCACTAT
GTTGCCCAGGCTGGTCTCAAACTCCTGGGTTCAAGAGATCTGCTGGCCCCAGCCTCCCAAAGCACTAGAATTACAGGCATGAACTACCTGTCTG
GCCCATTGTGATTTTTTTTTAATTCTCAAATTTGATTTTCTTATGGTTCTAAGACTCTCCATCTATGGGTCCATGCTTGAAGAACCATAAGATT
CTAAGATCAGAGGGTTCTACTGACTCAGTTCCCCTGCATCCTATAATTCTGTGTTTGGATGATTCCAGCATTTCATGGCCATTTCATGGGATCA
ACCAGGATTAACCACATGGCTAAAGGGAGAGGCTGATGGCCCTACTTCCATGGTTCCCAGATTCCATCTTCTCTTCCTCATAGGGAGGTCCACA
ATTCCCCACATATCACCAGGCCACCTGCAAGGAGAGCAGGTCCAAGAAGTCACCTTGCAATGAGAATGAGATCGTGTGCCTCCTACATCATGCC
CTAAGGGAGAGAGGACAGCATGGAGCTAGGAGGTGGATGGCTGGGGTGTCAACTGAGAAAGACATGCCTGTTGGACCCCAGAGTTGACTTTTAT
GGCATCCAAGAGCTGCCTGTGAGAACACACATGGTTTGATTTGTGAAGGGATGTTCACACAACCATCATGCGTCAGCCTTCTCAAAGGTGGCGT
TCGTAGCACAGCATGCGAAGGGCTTTGAGTCAACAGACATTCCTGGAAAGAAAGCAGTGTGGGGGTATGGAAGGATCCCTGGAGGCTGGGACCT
GGAGACCAGGGTCTTTCCTGCGGGCCCATTGCTCCTTCCACTTTGCCTTGAGCCCACTCTTTCCATACAAGGAGAGGCTGACCTAAGGTTCCAG
TTGCTTTCATCAGGTGGGATCTGGTGAAGGGTCAAAATGGAACTCTAAGATGGGTAGTTGAAGGGGTGACACCCGAGGAGAGAGTGATGTCTCA
AAACAGACTGAATTATTTCCCAAAGAGGTGCCCCAAGCCCCTGCAAATGCACTCTCTAGGAAGTCTGGCCACTGGGCCCCTTGCCCTCGTCCCA
CAGGGCAGCTCAGAAAGGCAAGCTCATGAGGACCGGCTCGTGTCCACCTACCCCCCTCATTGCCTACAAGGACTCCCGAGCATGCCCCCCATCC
TGGCGCAATCTTCTGCCCTGGAGCCAGGAAAGGATGGCCTTCTGAGTTGGAGTCCTGCAGTCGGTCTTCCAGTCTTGGAGGGAAACATATACTC
TACAAATGGTGAGCCTCGGAGAGAGCCCAGCCCTGCTGTGCTGCCTCCAGGAGGTTCAGGCGGCTATGCCTCCTCATTCAGCCTCACTGAGCCT
TTTCCACTTCCAGAACATGGTGAAATTCACTTCCCTCTGACCACACAGTGCTTGGTGGATCACCGGGCACACAGTGGGCCTACAGTGGGCATGG
TAGTCGCTGTCATCACTCCGCTCCAGGAGCCTTCAAGAAATCATCTCATCTGACAACCCACATTTACCATAAGGACCTTTTGGAGGCCAGGCGC
AGGGGCTCACCCCTGTAACCCCACAGTTTGGGAGGCCAAGGCAAGAGGATTGCTTGAGCCCAGGAGTTAGAAACTACAGTGAACTATGATGGCA
CCACTGCACTCCAGCCTGGGCGACGGAGTGAGACCCTGTCTCAAACAAATAAAAAAGAACCTTCCGGAGAACTGAAGGAAAGTGGAAATTTGAT
AAGAGATCTGGGCTGGAGAGGAGAAAAGTCTTTTAAAATATAAATATAACGGCAAGCTCAGAAAGATAGATACAGGGCAATGGTAAGAAGCAGA
CAGGTAACCAGATTGGGCTAGTGGGAGATGCTCATTGAGACAAGCAGTGCAGTGGTGTGCCACTGTGAGGGGGAAACGGGGATGGTGCCCCCGC
CATCCTGCTTTGTGACTGGCGTGCACGGCAAGGGGGCAGAGCAACAACTTCTGTGGGCTGGACCTGCCCTGGGCAGGGGCCACGGGGAGGCCAC
AGACTCCGGTCTGAATAAATACACAGCAGGAACCCAGAGCTGTGGATATGTAGACCCCACACACAGCCTCCCACAGAGGGCAGGTAACCCACAG
GAAGACAGACAGCCCTGAGCTCACAGCCATACCTGCAAAGCCCTGCCAGGACATACCCACATCAGTAATGTCCACGGGGGCTCTCATGGTGATA
TTAACGCGGAGGAGGTAATTCCCACAGTGCCTGAGCTGAACAATGCCAACAAAGGTGGAGACGGGGCGGAGGGACAGACAAGGAATGTAGAGAG
GGGCTGCAGACAGGAAGAGCAACTCACAACTGTGCAGGAGGGCTCATCGGCTGTGACAGGAAAACACACAGAGACTGATAGAAAGACAGGACAG
CCATAGACAGGAGGTGGGAGGCAGACAGGTCTGTGGCCCAGAGACCCTGTCTCACCACAGCAGCTTTGGGGAAGCAGGTGGCCACGTCATTTTC
CTTGTCTCTGTTTGGCTGACCTGTGCATCTCAGGCCCAGCCCTGGCTGCTGTTTTTCCAGCTGCAGTTCCCAAACTGTGTGCTAAGGCACCCTG
GGACGCCACAGCAAATTCCCAGGGTGTTGCAGAATATCACGCCTGTAATCCTAGCTCTTTGGGAGGCCAAAGTGGGAGGATCACTTGAGCCCAG
GAGTTCAAGACCTGCCTGGGCAACATGGTAAGGCCCTCGTCCCTGCAAAAAAAATTTGTTTTAAAGAAAGTACAATTGAGTCTTGGACAACATG
GGGGTTAGGGGCACCAACCCCCTGCAAAGTCAAAAACTTGAGTATAACTTTTGACTCCCTGAAAACGAAACTACTAACAGCCTCCTGTTGACCA
GAAGCCTTGCTGATCCCACACACAGTCAGTCAGAACATACGCTGCATGCCACATGTGTTATCCCCTGTGTTCCTACAATAAACTCAGCTGGAGA
AAGGAAATTCCTGCTCAGAAAACCTAGAAAAGAAGCCAGACTCACAGTTCATGAGGCGGCAGTGGGTCCTCATAAAGGTCTTCATCCTAGTGGT
CTTCATATTGAGTGGCTGAGGAGGAAGACAGAAGCGGGGCTTGGTCTTGCTGTGTCAGGGTGGCAGAGGCAGGAGAAATCCATGTATAAGTGAC
CCACACAGTGCAAACCTGTGTTGTTTGAGGGTCACCTGTCCTGCATGAACATCACCTTGTATGGCCTGAGAGCCAGGACCTTGAACAAGAAGTG
AGAGTGTTGCTTCTGGAGGGCCAGTAACTGCAGTTATGTGAGGATGAAATAAAAATATGATTCATTTATTTTTAACTGATATGATTTTTTTTCA
AATAGCTACTGATCATTAAGTGGTTGGACCTAATTACTTAATAAACAAAGCTCTTAGGTATTTATTTTAGCCTGAGGAGCTGGGCAAAGAATGA
CTATAAGTTGTTTTCTTGATTAATTTAATTTAATTTAATTTAATTTTATTTAGAGTCAGGGTCTCACTCTGTCACCCAGGTTAGAGTGCAGTGA
TGCAATCATAGCTCACTGCAGCCTTGAACTCCTCAGCCCAGATGATCCTCCCATCTTGGCCTCCCAAAGTGCCGGGATTACAGGTGTGCACCAT
CACACCTGGCCAATTGAAAGTTTTGATCTCAAGAACCCACTGCTTATGGAGAAGGCATCCAAAGAGTGACCCCCACAAGGAAGGTCAGGGAAGC
TTCCAGAAGGGACCAAGTTCCAATGGAACTGACTGTGGAGGGGCACTCCATCTTCTGACCCTGGACCCCTACCCTGTCCTGCTGCTGTTCCTTC
CAGGGTCCCTCAGCCAGAAAGCCCCCAGAGCCTTGAACTCTGTCTGCCCAAACCCCTTCTCTGTTTTCCCCCAACTTATTTTTGCTCAAATGAG
AAAAAAAGGAAGCAAAAGATGACCAGTTAGTGTCTGAAATCCTGGCTTGCCCCACCCATAACAGGGACTCACACCCGCTGTAGACAGGGAACCC
ACTACCTCACACAGCAAACTGTCCATCAGAGATGCCCCTCCCACCTGGCTTACCCCACCCAAGCAGGTATCTTACAGAGCTTCCGGGGTGGGCA
GCGCCTCCTCTCGCCTGGGGGCCTATTCTGGAAGGAGGGAGCACGCTCCCTGGGCCCTCCAGCAGGCCAGAAATGATGTTCCCTCCAGCACCAT
CCATACATGGGGAGTTTTGTTTTATCAAACACATGGCTGGCTGCTCAGGGCTTAGCTCCAGAGCTGCCCGGTTGTCAGGCCTTCTCACACGTGC
CCTCTCTGAGGACGATTAGCCCTTTCCTGACGCTGGCTTGCAAAGGTCTCACCCACCTCCCCGTTCCAAGTGTTGGAATGTGGGTCTGTGTCCT
GGCCAACAAGGAGGTCTGTGGAGACAGGACCGGGTCTCAGTCACCGTTCAGCAGCCACCCGGCATGCCCCCAGGAGCCTGCCCTATGCCTTGAC
CTCAACCTACGCCATCCCAGTCCCCAGCGCAATGCACAGGTGGGGACATAGACCTTGACAGACTGGTCAGGGACAGCTCCCTTTAGGCAACAGC
AGAGGGCGCTTCCAGGGAGGCCCCTCACCTTCCTCTGCACCCCAAGCAAGGGACAAGAGGTGCTGTAGGCAACATCACCCGAGCCTGGAAGACA
CTCAGGAGAAGCCCCTCAGGCCCCTGACACCCAGCTGGGGTCCCACCCTTGCTCTCCCACTCTGCTCTCCACCCCACCCCAGCCCAGCCCCACT
GACTTGGCTGCTCAGGAGCCGAGGCTCTCGGAGGCTCACAGAGGGAAAGAGGAGCCCCACATGGTACAGGCAGGTACCGAGGAGCTAGTTCCAG
AAGCCTCCACTCGTTCCTCCATGCAGCCTACCCTGGCTCCCTGGTATCCAGAGTCTTCAGAAACAGGAGCCCTCGCTTGAGAGCCTGGCCTGGG
CAGGCTGAGCTCCTGCCTGGGGCCTCGGAGTCTGCTCCTCCCTTCATCTTCTGCAGCCTGGGAAGCGGCCGGATCTCAGTGTGTCTACTACCCA
GGTGGCACGGGCAGAGGATGGGGCCTGATGGGGGAGAAGAAAGCAGCCAGGAGGAGGGGCTTGGGAAACCAAGGGGCAAGAGGGCAGGACTGAG
CAGGCTGCGCTCACCTGCCCCCTTCAGAGAGGTCACGAGGCCCATCCTGTCGGTGGGCAGAGCTGGCATCCTCTAGGGGGCTCTAATCCCCGAG
CCTCATTTCTGGGGAGGTGGGGGTAACAAGAGTCTGCGTGCCTCAGGCTGGGCTCAGGCGCCCACCCCACCTGCAGCCCAGGTTTTCCCCAGTA
CCACAATTGGGTGGGATTTTTGCTGCAAGGCCAAGTCCAGAGGAGCACAAACACGCTCCTCCCCTCCCCCTCCCTGACCCCACCGCCGCTTCAT
TCCAGAGCTCTTCCCTCTTTCCCTCTGAGGGTGGGGGCCCCGCAGTGCCTGGGAGAAGCGGGTGGGCGGCCGGGCATGCGTAGCGGAAACTGGA
TTCCCAAGGTAGTGAGGAGCAAAGGGAACTTTTTTTCAAAGAGAAAGGCTGTCACTCTGAATTCAGGACTCCTGACATAGTCACCAGGTGACCC
CTAGGGCCGGTGTCCACAGGACCACAGGCTCCCTCTGCAAACGCAGAACCAAGTTGTGAGTGGGGCACACTGTGGGAAGCAGCTGCCTCCATCA
GCCAGCCAGTGACACACCTGGGGACATCTCAGCCACCCCCTCCCTGAGAAGCTGCACAGAAGCGGCCACATGCCCATCCCTGCCTCTCCATGCA
CCTCCCTCCCGCAACTCTGCAGCCGCACAGGAGGCCTAGCCCACAGGCTGGGCAGTGCAGTATCCCCAGAGAACACACCACGGATGAGTCTGGA
GTTGCTTGGAGCTCTCAGAAGCTCCCGGAGCCTCCTTCCTCAATTCCAACGCGGTAACCAAGATTCCTGCTGAGCTTAGGTCACAGTGTGACTG
CAGGAGGACCAGCCGCGATGGGAGAGTGCCTCTTAACCATCCCAGTTATCAGGTCCCCCGGGGGAGGCTGGGGACACTTCCCCTCTGCAAATTA
CAATCTCCCCAGGCGGCTCCCGGGGCAACACATGGATATGCAAATGACTGGGAGCTGGAGAGTTGACTGGTCACTGGGTGACCTTGGGCAAGTC
ACTTTCCCTCTCAGGTCTTCCCCTCTCCATCAGCAAACTCCTAAAGCTTAACTAAACAGTCAGAAATCCCTCTGTCGGAACAGGCTGCATAGCT
CTGGGCAGACCTGGAGGTTGGTGGTCCCAGAACCTGGGGGCAGCACTGCGCTTCCCCACCGTCCCATCGCGTCAGGGGCTTCCACTGTTCTCTT
CCCTGGTGGAGTGATGGGGAGAAAAATGGAGGGGTCAATCCCTGCGCCACAGAGAGAGTCCCACTCGTCCCCAGCTGTGACCCACAGACGCGTG
GCTGCACCTTGGAGGTCCACCTGGCCGCTGTCCCCATTGCAGGCTGCACCCATCCCGGACCCTTTCTATCCGCCGAAGGCGCAGGCAGCTCTGG
TCTCAGCGGAAGGAGTCCGGCCGCAGGCAGACGGGGCCAAAGGACGTGCACCCGACAACCGTCCGCTCCCTCCCCAAACCCCCGCCCCGAGAAG
GTTGATGGATGAAGGGGAGCCCTAGCCCAGCCACCTCGGGGGCAAGAGACGGAGCTCGCCACAGACCAGGAGCGAGAGGGGGACTGCGGGTCCC
ACCGTGAGCGCAGGGCGCGCGGGGCTGGAAACCCAGGACGCGGCCCGGGAGCTCCAGGAGGGCGCCTGCGGATCGCGCGGCCCCGGGCCGGCCG
CCTGCCCATCTGGCGCACCCCAGCGCGCCGCGCACACCTGGGGGCCCGCACACCAGCACGTCCTCAGACACACCGACACCCACGGCAGCAGGCG
GGGGCCAACGCCGCCGCCGCGGTTCGGGCTCCCGTGGCCCCGCCTGCCCCTGCCTGCCCCGCCCGGGCCCCGGCCCCGGCGGGCGCCCCCCGCG
GCGCTGCCCCGCCCCCGCCGCCCCGCGCCGCCCCGGGCCTCGGCCCGCAGCCCGGCCGCCGGCCCACCTGGCGCAGCGCCGCCCTCGGAGCCCG
CGCACACCCGCGCACCCGCGGCCGCAGGAGGGCCCAGCGACGCCGCCGCGCCAGCTCCCAGGGCCCGGCCCCCCCCGGCGCTCACGCTCTCGGG
GCGGACTCCCGGCCCTCCGCGCCCTCTCGCGCGGCGATGGCCCCACTCGGATACTTCTTACTCCTCTGCAGCCTGAAGCAGGCTCTGGGCAGCT
ACCCGATCTGGTGGTGAGTGAGCCTCCTCGCGTTCGCCCCTGCCCCTGTGCGCCGCGCCCGCAGCAGACGGTCCCCTCGGGCAGGGACCCCGCG
GTGGCCCGAGCCCGCGCCCTTCTGCTCCAGCCCCGCGTGCGGGCCGCGGGCGGTTGGTTTTCCTTGACGGCCACTTTGGACCTGTTCAGGCCGC
TGGGACGCGTGGGTGGCGGAGTTTCCGGAGACATTGATTCCCGAGCGGGGGAGTAGGGGAGGTTGCGGAGGTCCTAGCTGCCCCTGGCGTCGGA
CAGGGCGAGCAGTGGGCTGGGGAGAGGCTGAAAGAAGGTCGGCACGCACGGTCACCGAGGGGACAGGCAGCTCGAGGCCAGGGGACAGGCGAGG
GAAGAGGGCCAGCGAGAGAGAATTCGCCGGAGACTTTCCTCAAAAACCGTATCTTACACACAAACACACACACACGTTTTAAAACAAAGTCGAT
GAGACAAGACTGAACAGCTCGGAGCAGCGTGGTTACGTAAAGAAAGCTGGGACCCGGCGGGGAGTGGCGCAGAGCCGGCGGCGGGGCGCACTGG
GGGCCGGCCCTGGGCCCCGCGCGCCTCCCCGCCGCAGTCCGGGCACGGGGGTTTCCAGGGGCCCTCTCTGCGAGCCCTCCGCATGGGTCCACCT
GGCAATGAGGGGCTGCTGTAGAGAGAGTTAAGGGTGAGTTAAGCACGGGGTGTGAGGGGCTCCAGGACCCTCAATCAGAAAGCGCTGTGCTGCG
CCCTCCACACCAGAAAAGGCGCGTTCCGTGAGACCCTCCCCAGCCTGGCGATGGAAGTGCAGATAAACCAAAGGAAGGGTCCCGAAAGGTCTCT
CTCAGGCCTCCCACCTCCACTGCACATATCCTGTGGGAGGGGGAACGGTGGCCACACTTTCGCCAGGGCTTGTGATCCCTCAGAGCCCTCACCA
AGCAAGGATCACCCCAGTTCCGAATTAAGGGCGCTCTGAGATGCCCAAGATTGAGGAACACAAGTGGGAGGAATGTGGGCGCGCAGGGCCGGGC
GCAGCGCTGGTAGGACCCACAGTTGGAGAGAAGCCTCCCCACAAGCATGCACACTCCCCCCCATCCTTCTCCGACGGCAGCCTGGAATTATAAT
AATTACGCCGAGGGGAAGGGGGGAGACCCAGCGAGCCGAGGTACATCTAATCCGATAATAATTTTTCTCTTCGAGATGGTTCAGGAGCGGGGGC
TCCCGCGGTAGGGGCGCCGGGCCAGGAGCAGCGGGGAGGGGAAGGGGCAGCCCTAGGCAGCTGAAGGGCCTGGAAGAGGCCACGAGGCCGCGGG
GACGCGCTTCGGGGACCCCCGCGCGCCCCTATTTCCGCGTGCCCCATTTCCCGTGCGGCACCTGCTGCGCCCAGTGGCCCACCTGAGGGTCAGG
CTCGGGGCTCGCCTTGGGGTGCGGGGATACTGACGCGCGTCCAGACGGCCGCAGGGAGCCAGGGGCAGCGCGTCCGTCCGAGAGAGCCCCGAGG
GCTGCCGGCTTACGCACCAGGCTTCTAATTAGAACCCGCCGCCGCGTTCCTGAGCGCAAAAGGAAGCTCCTTCGCCCCGCGCCCTTCCCATAAA
TGTCGCCAGCCTCCTGGCAACCCCGCGCCCTAGGATCTTTGTCTTGTCGGCCCTGAGGGTTCCTAGCCTCGGTCCCCATCCACCTCATATGCAT
GTATACCTCCTTTTCACCTGGGCCCCCTGTGTTCTACCCTGAATGGACCCTGAGACACCCTCGGGGACAGTCAGGCATTCTGCAGGGGAGCCAG
GAGGAGCAGAGAAGTGAAGTCCCTGTACCCTCCGCACCCTCCTATCAAGAGTCCAGGACAGCCCTTTGGCCACACAGGGAAGCGGGGCATAGAA
CCCCACCTGACACTGACCGGGGTGGGTGGTGAGGACCCCTCCCCCATCCCCACCACCACTTTGTTCTCAGTAGGGTTCTCACACTTGGGGATTC
TCTGCTTCCTGAGAAGCAGCCTCCCCTTGGGGGAGGCTGGCTCCCTGGGGACAAGTCTGCCCCCTTCTTGATCCCCTCTCTGCTCCCAGGCAGG
CTGCTCGGGCTGGAGCAAAGACATGGCTTCCCCCAGGCATGAGCAGCACCCCGGCAGCATAGGGAAGAAGACTCACAGAAGGACGCAGTGCTGC
AGGACCCCTGCTGCTGGACCCCTGCCGCTGGCATCCTACAGTTTAATAGCCTTAATAGCCGGGGAAGATTCTCTCTCTTTCTCTGGTCTTCTAC
CCAGTAGAGCATCTCCTCCTTTCTCCTGCCTCCCCGCATCCACCCCAGATTAAGCATCACCAGGGGAGGAGGGGACCTTGGCCCCTGCCAGGTG
GGCTCCTGGTGAGCCCCAGTGGAACAGAGCAGGCTGGGGTGGGGGGCCTCCCTGTAACTCCTCACCTGTGCCCCCAGCCCCTCCCTAACATGAA
GCCGCTGGCCCCTTGATTCTCCCCCCTCCACCATCACCGGCACATGAAAGTGAAAGTGAAGGGGAATCTCCTCACCTGGAGAAACAAACCAGGA
AACAGCCCAAATCAGGGTAATTAGGCTTTTGAAGTTGGCAGAGGGAGAGGCAAAGGTGTCCCTGTGGCCAGCAGCCCTGTGAGAGTGTGAAGGG
CCACCTGCCTCCCCAGGGCTGGGTGGCCCAGAGGTGTCTTATACCCACGCAGCAGATGCCAGTGGTCCCAAGGCTGAGCGGGTCTGAAATGCAG
AGAGACGGGCCTAGCCTAACACTGAATGGCCCAGAGCTGCACAGATGTCCAGGGGCATCCATTCTGAGCTCATCCAGTGACAGAGAGGAGACAG
GCAGGAGACCAGACCAATGCATATGCATCTGCCCACCTGCGTCCCATGAGGCCACAGACGGCAGGAACCTAGTGGCCCCAGCCTCCCAGCACGC
CCCTTCTAAGCTGAGTTCTCAGGAACAAGAATTCTTGGAAGAGCCCACATGGACAGAGGACAGGGCAGCTGCGCTGCAAGGAGACCATGGCTCC
ATCTGAGAACAATCCCCAGGCACACCCGCACTCCAGGGCACTCTGAGCACCTCTCCCTTCTTCCCATGTCTGGGTTAACGGAATCTTGTCATCT
CCCCAGGGACCCCACCAGGACCCTAGAGGTGGGCCTGTGCCTCCCGCCACCGCCTCCCCAGGGTCCAAGCCCACTTCAGGGCTTGCAACTGGAT
TTTGTTCATCTTTCAAGTCCCTCTGCTGCCTTTACCAGACTGGGCTGTGGCACTCCCGCCACGGCCACCTCCTGCTTCCGTTCTCAGTCCTGGC
CCTCCCCTGGGACCCACACCCTCCCAGCCACCTGGAGTTGCTGCCAGGCCTGGCCATAGCCACCGTCGCTGCCTAGTGGAGCACAGCAAGCTCC
ACCCCTTACCCAGCCCTGCTCTCCCTCTCAGGCCGTTCCTTCTGCCCGGCCGTCACCAGCTCCTGCTGGACTATGGGATCCCAAGCACAGAGGC
CTGGCTGCTTGCAGATGGCCACCCCAGTTGTCCCAGCGAAGTGCAGTGTGGGCTGTGTCCAGCCTTTTGCCTGCCCCTGGCCTGCTCCACAGCC
TCGCTCATGCTCCCTCCAGGGTCTCCCTCACATCCCTCTGGGCCTCGAGCTTGTCACTATGACAGAAACACATCATGACCTCCATTTTTATAGG
GCACCTCACACAGCCCTCAACAGAAGATGTAGGCCCTTTCAGGCCCCCACTCTCCTACACACTCATGGGTGCAAACAACCTCCTGGGCACAAGA
GCTCAGCCCCTCCTCCTAGCCCTTCATGGGCTGGAGGGCCAGCACACTTGGAGGCCCTGCTCAGCCAGTGTCTGCACTGCACCACCGCCAGGCA
CTTCGCCCCTGTGTCCTCAGTCCTGGTGGGCCTCATTGTGGTGGGGAGACAGCCTGACTGGAAGAGGGGTCGGGTGAAGAGGCAGCTTAGAGAG
GCAGGAGCCAGGAAAGGGTGCAGGAGAGAAGGAGCTCGGCTGGAATGATGGGTCAGGCATACGTGTGTGGTGTGTGGGCAGGTGGCTGCAGCAT
GGGTGTTTCGGGGACTGAGAGGGGCCAGTCGAGCAGGGCAGGGACATGGCCCAGGGTACCCAGAAGCTCAGATGGGAAGGGCCTTGTGATAGGA
GGGATGGTTGCCACTCCCAGAGTCACCCTGCCTCCACATTAGCCTGCCACTGGCCCACTCCCTCTGCAGGCCTGGCCTCACCCTAGCCCCTTCT
TCCTTCTCCCTTCGCCCGCATCTGTCTGTGTCTCTCTGTCTCTGTTTCTCTCTTCCCTGCCTCTGTCCCTCCATATCTCCATACCTTTGTCTCT
CAGTTTCTCCCTGTCTGTCTCTCTCTCTTCCCTGTATCTGTCTCTGCCCGTCTCTGTCTCTATCTCTCTGTGTCTGTCTCTCTCTTTCAGTCTT
TGTCCTGTCTCTCTCTGCCTGTTTCTGTCACTCTATCTCTTTTCATCTCTGTCTTTCTGTCTTTTGCAGTCTCTGCCTCTCGTCTCTGCCTCTG
GTGCTGAGGACAGCAGTCACTAGCAAATAAAACTCGTGAAACGAGCTGAGTGGAGCCACCAGGCCAGCCCTCCCAGAGCTGTGGGATCTCAGGG
CCCTGGGGGTGGCATTGCTTCTCAGAGCTGCCCACACACAAACCCCTCCTGAGGGTGACGGTCAGGAGTGGCCACACGTCAAGTGTCTGCAACC
AGACATTGGCCCTGCATAACTGCCAGGATCCGTAGCCGCCTCAGGGACGCACAGCACCCTTCCAGAGTGCAAAGCTCCTCCTGCCTGGTGCTCG
CACCTCAGGCACTGGCCAGCTTGGGCCCTTGAGGCCAGCTGCCCCTCTGACTCACGGGGCTTGTTCTAACGATGTTTGCTTCGCATAAGCCTCC
TAGAATCTTTTGTAGCAGGGAGAGCAGGATGAGTAGATACAGTCATAGCAGCCCAGTCTTAGGGGTGGTGACTGCCCGGTGGCCACTTCTTCCA
CTGGGTCCCTTGTGCACATCACCGTACTGTGAGGAGGGAAGCTCCGGTGTCCAGCAGCCCTCCCAAGATACAGGTGACAGGGCTCCCGCCATCT
CCATCCAGGGACCCCGTTGGGGCTTCTCAATGTGGGGCCTACCATGCAGAGCCCAAATGTGAATGCTTGGCCACCTGCCATGAAGAAATCAGAA
CAGAAATGGGAATTTAAAAAGGATTTGGTGACATTGTAGCGGTTTGACGGTGCTGCAACCATTTCTGTGGATTTTGTAATAATGGAAATTTAAA
AACAAACCAACCACGTCCCTCTGTTGGGACTCTAGTCTGAGAAAGCCAGCCTGAAGCCACACCTGGAGTAATGGGGGTGGGGGGGCTCTCTCAA
TGTGGACCACTGCCTGTAGCCCTCATCCATTGGATTTTTGACCCAAACGTGAGACTTCTAACTTGCCCCCTTTAAGAGTAGTTGAAGGGCTGGT
TGCTGTTTCTGTTCAGAAGACCCGCTTCATGTCTCTGAAGGCTATTGGCTGGGGAAGAAGGATTAGACTGTGGCTCAAGGGGCAGGGGCTGGGG
CAGAGGTTGCAGGTGCCAAGGAGGCTGGTTCTGGCTCCCAAAGAGAAAAAGCTCCCTAGGGATGAGAGCCTGTGCCCCAAGTGGTACCAGGGAG
GTGCTGAGCTGCAGCTACCGAGGCCGAAGAAAAGGAATGTCATTCACAAAGGAGACCTGTACAGCCCCACAGACAGGGTCTCGCTCTGTCACCC
AGGCTGGAGTGCAGTGATGTGATCTTGGCTCACTGCAGCCTCGACCCTCATGGGCTCAAGTGATCCTCCCATCTCAGCCTCCTGAGTAGCTGGG
ACCACAGGCGAGCACCACCACTCCCAGCTAATTTTTGTATATTTGTAGAGATGGGTTTCTCTCTCTACAAGAAACCATTTCTCTCTCTAGAAGA
AACCATGTGGCCCAGGCTGGTCTCGACCTCCTGGGCTCCAGCAGTCCTCCCATCTTGGCCTTGTGAGTAGCTGGGATTACAGGCTCGTGCCACT
ACGCTTGGCTAGTATTTTTCATTTTTTGTTGAGATGGGGTCTTGCTCCGTGGCCCAGGCTGTGCACTTTAATGTTGGACAATCCCCCACCCCAC
CCCTCACTGGTTACAGCCCTGAGGCTGGTGAGGGGCACATGGCCTCTTCCTTGGCCTCCCTACTGGCCTCCCCAGGTGACACCCTGGAGGTGTC
TCTCACGTTGCTGCTTCCACATTCCTTCCCCGGCAAAACGTGCCGCTGCCGCCCAGGGAAACTGGCAAGTGCGTGGCCGCGGGCACAGGAGCCG
CTGTGCCTGCTGGGACTCGTTCAAAGTATGTGTCAGTAATCCCAGCTGGGCCGGCGGGCAGGAGGAGGTGCGGGTTGGGAGAGCTCCAGGCAGG
GCTGGGCAGCCTGGACTGGGAAGGGTTGCCGACACCCAGGAGGGACCTGGGGGCCTTAAGTCCTGGGGACCCTGGCCGGCTCACAAGGTGCTTG
AGCTGCCGAGAGTGGTCTTCTAGGCAGCTGCAGTTCCACCACTGGGTGTGAGAAGAAACCAGAACCTTGGGGGTCTCCCAGGCAGGTGGGTGCC
CTCGGGCAGTCTCAGCCTGGGCGAGATGGGAGGTCATCCACCAACAGGGAAACACTTCGAGCCTCAGGTCTGAGGAAGGAAGCCTTATCACCGC
CCATGGAGTGGCCCTGATCTTAAGAAGACAAAGATTTGGAACAGGATGTTTGCTCCAGAGGCCCCACAGAGGGGAAGCCCACCTGCCCAAGCAG
GACCCTCAATGGCAGAGACACCCACCTGCCCTGGTCCTCCACACCTGCCTGCATTGCAGCTGTGCTGGAGCCAGAGGGGTCCCAGGAGGCGACC
AGCTGGACTCAGGGCACAAATTGCATCCTGGAGGGGTGGCCCTGGCAGGTCCGCGAGTGGGGTGCACTGGGTCCCTCTCAGGCCAGGAGGAGGC
TGAGATGACATCCAGCAGGGCAGCAGGGCCTGAGATCAGTGTCCCCATTCCATGCACTGCTCCCCTCCTCTCTCAGAGCTTCAGGTAGGAGAGG
GGTCCATTACCCCATTTGACAGATGGGGAAACTGAGGCCCAGGAGGGGAAAGAGAGAAAGAGCAAACAAACAAGCGAGTAACAGGGCCCGAGTT
GGAGCCCATGCCCACCTTCCAGGCCTGTCCACACGCCTGCATGGAGCATTACTGTGAGGTCCCCACGCCCCCAGCAAACATGCAAGCACAGAGC
CGTGCCCTTGGTACATGGGTGCTCTGAGTTCGCTAAATTTTACTGCTAAAAGACACCAGGCCTGGACTTGAGAAACCTAAGAGCCTGCCAGCCT
CAGAAGGACTTGCACAGGAACAGTGGAGGCTGGATATGGTGGCCACTCAGGACCCCCACAGCTGTGTGGCTGCGAGAAGGCCCCCACGCTTTGC
CCTGAGCTTCCATCCACAGGGATTAGGTAGCACCTCCAGTGCCGCCACGTAAGATGGGAAATTGCACATGGGACCAAACCAAGCCGGGGAGGCC
AGCGTGGCCCGGACTGGGCTGGCCTACCGCCCGCCTTGTGGTCCTGGGCCAGCCTCCTTCTTCCCCTGCATGTCACAGCCAGCATCTCTGCCGT
ACCTCTGGCGAGCTGCCCAGCCTTTCAACAGAAGTGAGGGAGTGGGGAAGAAAAACCATTAGTGCTTGTGGGCTGGAGGCATGGCTGCTGTGAC
TTTGGCCACAGGCGGAAGCCATGGGTCTAGGGACTGGGGCCATTTTATAAAACTACATTTGTGTGTGTTCTGAGTGAATATGACAGCCTCCCCT
TCAAAACAAACGATTGTTAGGAAAAAAACAAAACAAAACAAAACAAAACCTAACAGCATTAGTGAAAATCTGGAAGGAAAGACAAGTCTGTGAT
CTTATCAACTTTTTCAACTTTTTGTTTTTCTAAGGATAGAAACAGTTTGTGTTCTGTTTTTGAAAACTCACTAAGCATCATCTCCATTTCTGGG
GCCTTCTGGATGATCACACGATGGTTCCACTAGGTCTTGTGGTGCTGGTACTGCGATCTGCTAGCCCAGCCCTGCCGTTCCCTCCAGAGCTGGG
ATTCCAGGGAAAGGGGACAGAGCAAGAGAGGCTGGAGATATTGGAAGAGCAGGGTCACATTCCCTTGGGACACCAATTTTGCACAGCCAGAGGG
ATATCTGGTGTCCCATCCAGTTGGGCAGTTAGAATTCCAGCCACTCAGCTGTGTGGCAGCTGGACGTGGGAAACATGGACTCTTCCTGGGACGC
TGTGGTGTTGGCAGTGAGAGGGGAGAGGGAATGAATTCCTCATCCGGTGAATCACAGGCTCGGGGTGCAGGGCATGGGGGCAGGGAGGGCAGGC
TGGCCTCAGCCTCTCCCCGGGGACTGGGCCCAGCCTTCCTGCCCCTGCTGTTCCCACCACCCTGAGGCAGGGCCCAGACCTCACTGTGATCCAG
ATCGTTGGCATCCAGGAAGTGAGTAGGGAGTGACGGATGGCAAGACCACCACCCCCGCCTCCCCCCCACCCCCGCCGCCCAAGTGCTTACCTGT
GGTTCCTTGTGTGAGTGATCCTCAGCAAGGACCCTTCGTCCTTGACCAGACACTTGTTTGCATGCCTACAAAGCGTGGGAGAGGTCGAAGGGGA
GATTGGAGAATTCATCAGAAGCCCCAGAAGCCCAAGTGGGAGCTCCTGGCCCTGCCTGCTGGCTGGGGCCACGCTTGTCTTAGGGCACCAGGCA
GGACAGTGGCCAATCGTGGCTGCTCCGCCTGAGGCCATGAGCAGGCAGGAGCCACGGACTGACAGCCAGTGCTCAGGGTCTGTCTGTGCCCGTC
ACCTTCTGGGCACCCGATGTTGCCCCTTTCTGTCCCCCGCATGTGCTTTAAGCGCCTGCTACTACCTTGCTCTTTTGGGGATCCTACTGACTAG
AGGGTGGAGGTAGGTCCCTGACCCCCCAGGGTGATTATCAGCAAGGCTGACTCCCCAGGGACTCAACCCAAGGGAGCCCCCAAGGACCCTCAGA
GTCCTCATGAAGACAGACAGGGGCCATGGAGCAAGTTTAGACTCAAGGAAGCCATAGAAGTTTCCCCGTCAGCCTTAGAATGTCCAAGCTAGAG
GCAGCCTGGGTGAACCCAGCACACATTCCCTGTGGCACTCACGCTCCTGGGGCCACCCTGGGAGGACCCTGTTCTAAACCACAACTGCCCAGTT
CAGTTGGGGAAACTGAGGTTCCAACAGTCCCAAAGCTGCAGGTGCAACTGTTAGGGCCACCACCCATGTCCCCCTGGGTGGCCTCCTGGGGTGG
AAAGGGGGCTGCATTGGGGACAGGAAGCCCTATGATGTCTTGGGTCATCAGCTGCAAGGAGGCTGTCCAGGGTCTGTGTGACTTGCGCCATTTA
TCTCTGACTCATTTATAAATGTTATTTTGCAGGGACTGGGGTTTCATACGTGTACTTGTCCATTCTGCATTGCTATAAAGGAATGCCTGAGACT
GGAAAGAAAAAAGGTTTAATTGGCTTAGGGTTCTGCAGGCTGTACAAGCATGGCACCGGCATCTGCTGAGGCCTCAGGGAGCTTCCACTCATGG
TGGAAGTCAAAGGGGAGGTACGTGTGTCACACGCGGGAGAGGGGGCAGGGGAGGGTGCCAGGCTCCTTCTAACAACCAGCTCCTGCATGAACTC
AGAGGGAGAACTCCCTCATTACCGCAGGGAGGGCACTGTGCCATTCACGAGTGATCTGCGTCTGAGACCTAAATGGGTGGGGCCCACTAGGCCC
CACCTCCAACACTGGGGGTCGCATTTCAACATGAGATTTGGAGGGGACACACATCCAAACTATATCAACACATTTGAAGGGCATTGTAGGATCT
TAGAAAGCAGTGGCCCCTGGAGGCCAACATGCGGGAAGCCCTGTTCTGCTGTATTAGCCGGTGGCTGTGGCAAAGCTCCATTCCCTGAGCCTTG
GCTTCCTGCTCTGGGAGGTCCCAGTGCCACCTATCCAGGTCCTGTGGGTTTCAGGGAGCCACATAACTAGAGCCCGTGGGGAGGTCACCAGTGA
GCTCTGCCCAGCAGGTGGAGGCCAGGTAAGGGGCTGGGTGGTGGTAGTAAGAGGAGCGATGCTGGGTATTGGAAGGGAGAAACAACCAGCTTCA
GCAAGCCTCCCTTTCAGAGACAAGAGTGCTGGAAAGGTGGAGAGGCCGCAGGAGGAGGGAAGAGGAGGGACTAGCTCTCTAAAGACCACAGTGG
CCAGGCGCGGTGGCTCACACCTGTAATCTCAGCATTTTGGAAGGCCGAGGCGGGAGGATGGCTTGAGTCCAGGAGTTCAAGACCAGCCTGGGCA
ACATAGACAGACACCATCTCTACAAAAAATACAAAAGTTAACCGGGCATCATGGCACAGGCCTGTGGTTCCAACTACTTGGGAGGCTGAGGCTA
AGATGGGAGGATCCCTTGAGCCCTGTAGGTCAACGCTGCAGTGAGCCCTCAGCCTAGACGACAGAGCAAGACTTCATCTCTAAAAAAGTAAATT
AATAAAATTTAACAAATAATAAAGGCCATGGCAGTGGGCTGGCGGAGACAGCGGTGGCTCGGGCCCCTGGAGCTTGCATAGCAAGATGCTGGAG
GTGGCAGCCTGGCCTTCAGATCCTTCTCCTTCTCTGGGTCTGAGAAGCTTAGGTGTTGGAACTCAGAGGTGGTCAGGAAATGCCAGCATGAGCA
GGAAAGTGAGAGGGAGTATCTGCTGGCTCCCAGTGCTGCAAGACCCTATCTCTACAAAAAATACAAAGCAAGAGAACAGCACGGGGGCTTTGCC
ATTGGCTAACGCAGACTGACCTCCCCAGTTTGGGCATGAACATCCTTTTCACGTTCCAAGGTGGGATCCGGGAGTCCAGGTTGCATTGTATTTG
ATGCACCCTGAGAACTTGCGCTGAGAACAGGACCCTGGGGACACTCGATCTCGAAGACGGGGTTCTCATTCACTCTCCATTTGCAGAGCCCCTC
CCCCACTCACTGCAGCCAATACTCCCTGGCGAGACCCTGAGACAGGCCTGTCTGTCCCTTGTCCCAGGCAAGCCTGGCCCACAGGGTGTGGGAG
GAGGAAGTTACAGGTCATAGTTTCCCAAGCACTGATGGATTCCGGGCATCTGCATTCTCCTTGGATTAGAAATTTGGATCCTAGGAGCTTTTGG
GGGCTGGTGTGGGGATAGTGCCTGTGCAATTTAGATTTTTTTTTTTTTGAGACAGGGTCTCACTCTGTCACCCAGCCTGGTGTAATCAGAGCTC
ACTGTGGCCTTGTCCTCCTGGGCTCAAGTGATCCTCCCACCTCAGCCTCCCAAGCAGCGGGGACTACAGGTGTGTGCCACCACACCCAGCTAAC
CTTTGTATTTTTGTAGAGATGGGGTCTTGCTCTGTTGCCCAGATTGGTCTTGAACTCCTGGCCTCAAGCAATCCACCTGCCTCAGCCTTCCAAA
GTGCTGAGATTACAGGCATGAGCCACCATGCCCAGCCTTATATTTGATTTTCTTTTTAATGACCCAACTGATCCTTCAAGGCCCAGCCAAAATC
TGGTGGCCCAGTGAGCCTCCAGGGCCCTCCTCTGCACTGCTCCCTGGCCCCACAGGAGCTCCCCGCTGCCTGCCAGCTTTCCACCCTCCTCAGG
CAGGGTCTGTGTGGCCCAGGCCTTCTGGTGGCCTGGTGCTATCAGAAGGAGAAATCTCTCAGGAAGCAATTCAACGAACTGGAGGGGCCCTCTG
GTCCACAGGAGGCAGGAGAAGCCCATCCTGACCCCAAATCCACAAGGGCCCGTCAAGGGAGGGGTCCCATCAGGGCTGTGAGAGGCCAGGGATG
CTGGAGCCACCTCTGGCCTGGCAGCTGGCCACCTGCACCCTGGCCATACTCAGGAAGGTGGTGGGCCAGGGCCCACCCGGGAGGAGGGTGAGAG
GGGCACAGAACTGGCTGACTCAGCACCCTGAGAATGGAGGCTTCAGACTTGGGAGTCCACCTCTCTGCTTTATCTAGAGACCAGGGACAAGGCC
GCCCCTCCCAGCCCCGAGCCTGCGAGCTGAGGTGGTCCCGGCTCCATGCTGGTCATAGTGTTCTGGGGGTGGGCAGAGTGACAGCCCCCAAAAT
GAATCCAGCATCTAGCCTCGGAGAAGGGATGAGCCTTGGAAATGGCCGCATCCCCCAGGCCAGTCCCTGTTTTGTAAGCAGCAAGCTCTAAGTG
GATTCCTTTGTTTCTCCTCATTCTAAGTCTCCAAGTAAAAAGGACATTCAACTACTCTTGCTTTTCCTTCTTTCAGACACCTCTTGCTCACTTC
CCTGGCTGTAAGCTAGGCTGGCCCTGGAGCCTGGGTACCCGGCCCTTCTCAGACACACTCCCAGGCCTCAGCCATGACACTCAAGTGTTTGTCC
TCTACAACATTCCATTAGGAAAATTTTCAAACATCTGGAAAAGATGCAGCCTTTGGACATCAAAGCCTTGTGTGTCCACAGCTCACACTGAAGT
CAGCCTGCTTTATCTCACACCCACCACCCTCTCCATTCTCTATCTATCCGTCTTATCTTTTTATGCTTACGTCAGTCTGCTCTGTCCCAAATAC
TGTAGCAGGCACGTCCTGAACCAGTGCTGGATGTTTTGCAAGTAACATCCCACATGGAGTCACACCATAGAATGGGTGAAGGGGAAGCATCTGG
GTTCATGGAATGGAGGCACCTCACCTGGGGCAACCACCTCACCCTGGCTTAAAAATGGAAGCCCCTTGCCTGGGCACGGTGGCTCATGCCTATA
ATCCCAGCACTTTGGGAGGCCAAGGCAGGTGGATCACCTGAGGTCAGGAGTTTGAGACCAGCCTGGCCAACATGGCAAAACCCCGTCTACTAAA
AATACAAACATTAGCCAGGCATGGTGGTGCACACCTGTAACCTCAGCTACTCGGGAGGGTGAGGCAGGAGAATCGCTTGAGCCAAGAGGGGGAG
GTTGCAGTGAGCTGAGATCACACCACTGCACTCCAGCCTGGGCAACAGAGCGAGACTCCGTCTCAAAACAAACAAAAAAGGAAGCCCCTGCAGT
CCTGGGCGAAGCAGACAGTTGGTCTCCCGGCCTCACCCATGGGCACCCCCAGGCCCCTCTGCTGGCGTTCCTGGGTGCTCTGAGGTGCATGGTT
TGAAAACCACGAGTCCAGCCCCTACGGTCACCTCCCTGGCCTGGTTCAACCCACCTTCTCCTGTCCAGAGGCCTCTCACCCACAGTCCAGGTCA
CAGACAGCTGACCTTGGTCACCACCACTTGGCCCTGGGCAGTGGCAGTGCAGACCATACCCCAGGGACCCCGGATGGTCATCCAGTTGCCGGGA
CTCAAAGGAAGAGAGAGGATGAGGAAGGAGACATGGAAAAAGACTACCCAGCTACCTACAGCAGGAACACCAACCCAGGCTGCATCAGCACTTC
CTGGAAGTGCTGGGATCTACAAAATGGTGGAGGGGGTGTGCAGGGACACCTGGGGCAGGATACCTGCGTGTAGTGAATGAATAAAGAAGTGAAC
AAGGGAGCCGACTGTGCAAGGACAGACTGCAGGGGTGCAGGCTTGAGTGGTGGCCTGGGGATGCACGTGGGGTCTGGCGCGAGGTTATGCACTC
AAATGAGGGACAGACAGGAGGGAGGGTGACTAGGGAGAGGGAGGCCAGATCTCCAGGGGGTAGACATATTGGGGACCCTGAACACTGGGAGTCC
GGTCCGGAGGCCAGACTGGTTGGATCCAGTGGACACATCGACAGGTGGTCATAAGCATAAGGAACTCTCTCCCTCCACCCACCTCCACCAGTGC
CCAAAAGGACCTGGTGATTTCTTTAAATTGAGGTGTATTTTGTACAGCAAAATGGAAGACCCTAAGTGCATGGCTCAATTCGTTCTGACGTGAA
GCACTCACATAACCAACATCGCAGTCAAACGCAGAGCACTTCGGCCAGGCTGGCCTGGCAGAGCTTCCCTGATGACAGGCACATTCCCTATCTA
ACTGTCCCATTCAGGGTAGCCACGGGCCACACGTGAAGCATGGCTGGTGCCACCGAAGGGCTGAGTTTTACATTTCTTGCATTTTAATTAATTT
TAAACTTAATTACATTAATTTAAACGGCACAGGTGTCGAATGCCTTTTGCCAAAAGCAGAGCACAGCCTGGGAGCTGTTGGGACCTGGAAAACG
AGGAGTGAGGTTGCCACGGTGCTGACCTCTCCCGCGGATGAGTGTTGCCTGGTCTTGAGCTCAGACTGGGGGATGCGGAGGGCCCCAGGAGCAT
CTCACAGTTGAGCCCCTCCTCCCCACGTGCTCACGGGAGCCCATGCCTTCGGGTTTCTGAGCAGCCTGGCACCCTAGAGGCAGTCTTCATTTGT
TTATTCACTCTCCTATTGACGGATGCTTGGGTGGCGTCCAAGTTCCTGCTCTCAGGCTCAAAGGCCTGGGAAGATTCCTGAACACGTCTCATTG
TGGACACGTGTTTACATCTAGGGGTACATAGCCAAAAGTGGGCCTGCGGGCTGGCGGGATGGGTCTGAGTTCCACAGTGATTTCTGCGCTGGGT
CACGCCTACATCCCGGAAGCCAGGCCTCCTCCTCCAATCCCAAAGCCAAGGGCATCCCTTCTCTGGGCAGCCCCCACCTGACCCGGTCCCCAGC
CTGAGGTTCTCAGGTTCTAGCCCTGAGTCCCCCATTGGGCTCCAGGTCAACCTGGTGTGAACGGGGCCCATGCAGTTGTGCTGAGCAGGAATGC
GGGGCTGCATCATTTCCTGTTACAGATCTGGACGACTTGAATTCTGAGCTGAGTTTTGGGGATAGGGCAGGTCAGGGTCCTGGCCTTGTAAAAT
CAGACTTCCAGATAAAACATACAGAAAAACACATAGATGATATCAAAACATCTAGAAAAAATTAAATAGCCAGCATTGAGTATTTATTCTGCAC
ACAACATTGTATTAAGCACTTCCTGTATAACATACAGGCTTAAGAGCTGGGCAAGGTGGTTCATGCCTGTAATCCCAGCACTTTGGGAGGCTCT
AGCGAGAGGTCACTTGAGGCCAGGAGTTTGGGACCAGCCTGGGCAACATAGCAGGACCTTGTCTCTACAAACTGTAAAAATAAAAATTAGCTGG
CTCTGGTGGTGTGTGCCTGTAGTCCCAGCTAATCTGGAGGCTGCAGTGAGCCATGATCATACCACTGCACTCCATCCTGGGCAACACAGTGAGA
TTCTGTCTCAAAAAAAGAAAAATTAGAAGGCATGGTTTATGAACATTATTTTAGAGGCCACAGAGAATCCTTAATTGGGCTTTTAAGTAAAAAT
TCTCTTACTTGCAAAATAAAAAATCAGATTTCCTTCATGGTGGAAGCTGCGTCTCGCCCCCCGAATGCACTTGAGGCCCCAGAGCAAAGGGTCT
GTAGCCTGCTCATCTGTGTCGCTGTCCCAGCCCCACACTCACCACCGGATCTTGCCCTCTGCAGGTCGCTGGCTGTTGGGCCACAGTATTCCTC
CCTGGGCTCGCAGCCCATCCTGTGTGCCAGCATCCCGGGCCTGGTCCCCAAGCAGCTCCGCTTCTGCAGGAACTACGTGGAGATCATGCCCAGC
GTGGCCGAGGGCATCAAGATTGGCATCCAGGAGTGCCAGCACCAGTTCCGCGGCCGCCGGTGGAACTGCACCACCGTCCACGACAGCCTGGCCA
TCTTCGGGCCCGTGCTGGACAAAGGTATGGGGGTGGTCTGGGGGAGGGCAGATGAGTCTGGAGTGGGACAGAGTCTCCCGCCTAGCGCTGCCTG
ACATTCTCATCTGTGCACACGGTGGGAACAGTGCCTCACGCGGACGCTGGGGTCCTTCCCGCTTACCTCCAGGAGGACGGTCTGGGGCTGAAGC
AGGACTGAGCCTGTGGGGTTATTCGGGTATCCCTCCTCCTCCTGCTCCCTGGAGAGGAAAGCGGGCATTCTAAGGTGACTCCAACCATGGCCAG
TGGCGGGGCTGCTCCCAGGAGCCAGGGCACAGGCCAGGCTTGCAGAGGAAATACACAAACTTAAAGACCTACAAGAAGGGAGAGGTTTGGGGGC
ACCCTAGAGAAGAAAGAGAGACAGAGAGAAAGACAGAGAGAGACAGAGAGAGAGAAAGATAGAGACAGACAGAGACACAGAGAGAGACAGAGAC
ACAGACACAGAGAGAGAGGAACAGTGAGACGGAGAAAGAGACAGAAAGATGCAGAGAGAGACAGAGAAAGACACACACACAGAGAGACAAAGAC
AGAGAGAGAGACAGAGAGGGACAGAGCGACCAAAAGAGAAAGACACAGAGGAATAGAAAGACAGAGACATAAAGATGCACAGAGACAGAGAGAG
ACAGAAATAGAGAGAGACAAAGAGGGACAGAGAGAGACACACAGAGAGAAACTGAGAGACAGAGAGACAGAGACGGAGAGAACATGGACAAGAT
TAAAACTTTAGCATCTTTTTTTAAAAAGTTGAGGTGAAATTCACATAACCGAAAATCCGCGATTTTAAAGTATTCAATTCAGTAGCATTTGGTA
TACTCACAGAATTGTGCAACCATCACCTGCCTAGTTCCAAAATATCTTTATCACCCAAAAAGGAAATCTGTGCGCATTAAGCAGCCATTCCCGT
TCTTCTCCCTTCCATAGCCCCTGGCACCCACCCGCCTGCCTTCTCCCCTGTGATTTAGCTACTCTGCCTGTTTAAGAAACGGCATCACTCAATA
TTTTGCGTCTGGCTTTTTTTCACTGAGTGTGTTGATTCACCTGCACTGTGGATAGTTTCAGCACTTCATTCCTGTTCATGGGCTGAATAATATT
CCATTGCATGGATGGAGCACACTGGTTTATTCGTTCTTCAGGTTGACGCATATGAGCGTGGTCTCCACCTTTTGGCTGCTGTGGCCTTTTCTTG
GACATGCATGTGTTTGAACGCCGTTTTCACACCTTTTGGTTTCTGTATCTAGGAGTGGAATTGCTGGGTCACAGGGTAATTCTGTTTCGCTTTG
CTTGACCTGGGAGGTCAAGGCTGCAATGGGCTGGAACCATCACACTATTTTCCACAGCAGTTGCACCATTTTATATTCCTACCAGTATTGTTTG
AGGGTTCCAGATTCATCCTCACTGATACACTGTTTTCTGGTGTTTGTATTTCTTACTTCTGGCCATCCTAGAGTGTGAAGTAGAACCTACTTCT
GATTTCAGTTTGCACTTCTCTGATGACCGATGATGCCAAGCACCATTTCATGGGCTTTGGGGCCATTTGTAGACCTTCTTTGAAATGTTTAGTC
AAGTCCTTTGCCCATTTTTAAATTGGATTCTTTGTCTTTTTGTTGTTGAGTTGTGAGTGTTCTTTATATATTCTGGATACTGGATGTGCATCAG
ATGTATGATTGGTGAATATTTCTCTCTCATCGTGTAGGTTTTTTTCATTTTTGTGAGAGTGTCCTTTGATACACAAAATTTTTTAATTTTGGTG
AAATCCAGTTTATATAGATGTCATACTGAAGCAAGAATTAGCTAACATAAGGTCACAAAGATTCACATCTATGTTTTCTTCAAAAAACCTAATA
GTTTATCTTTTACTTTGAAGTATTTAATCTATTTTGAGCTCATCTTTTTATATGGTATGAGGTAGGAATCTAACCACATTTTTTTGCATATGGA
AATTCAGTTATTCCAGTACCTTCTATGGAAAAAACTGTTCTTTTTCCCATCAAATGGTCTTGGCACCCTTGTTGAAAATCAATTGGTCATAGAT
GTATAGGTTTATTTCTGGACTCTCAATTCCAGTCTATTGGTCTGTATGTCTGTCCTGTCACCAATACCACATTATTTTGATTGTTGTAGCTTTG
TGGTATGTTTTGAAACTGGGAAGAGTAAGTTTTCCAGCTTTGTTCTTTCTTTTCCAGATTGTTTGGGGGGGACAGGAAAAAAGATTGTTTTGGC
TATTTGGGGTCCCTTTCAAATATATGTAAATTTTAGGAGCAGTTTTTCCATTTGTGCAAAAAAAAATGCTGCTGGGATTTTCATGGGGATTTCA
CTGATTCTGTAGGTTGCCTTGGGAAGCATTGCCTCCTTAACAATATTAAGTTATTTAATTCAGGAGCTCAGGGTCTTTCCATTTATTTAGGTCT
TCTTCCTTAATTTTGTTTGTTTGTTTGTTTTTGTTTTTGAGACAGGGTCTCACTCTGTTGCTCAGGCTGGAGTGCAGTGGCATGATCACGGCTC
ATTGCAGCCTTGACCTTCCAGGTTCAAGCGATCCTCCTGCTTCAGCCTCCCAAGTAGCAGGGACCACAGGCATGCGCCACCATGCCCTCCCAGT
GTTTGTATTTTTTGTAGAGATGGGATTTTGCTGTGTTTCCCAGACTGGTCTCTAACTCCTGGGCTCAAGCGATCCACCTGCCCTGGCCTCCCAA
AGTGTTGGGATTACAGGGCATGAGCCACTGTGCCCAGCTTCTGTAATTTCTTTCAGCCATGTTTTGTAGTTTTCAGGGTATGAGTTGTGTACCT
CCATGGTTAATGTATTCCTAAGTACTATTTTTCTTTTTGATGCTATTGTCAATGGAATCATTTTTCTTTTCTTCTTTTTGTTTTGAGACAGTGT
CTGGCTCTGTCACCTAGGCTGGAGTGCAGTGGCACCATCTCAGCTCACTGTACTTCCATCTCCCAGGCTCAAGTAATTCTCCTGCCTCAGCCTC
CTGAGTAGCCAAGCTTGGCTAATTTTTTGTATTTTTTTTTTTTTTTGTAGAGATGGAGGTCTCCCTATGTTGCCCAGGCTGGTCTTGAACTTTT
GGGCTCAAGCAATCCTCTCGCCTTGGCCTTCCAAAGTGCTGGGGTTACAGGCGTGAGCCACTGTGGCCAGCTTGTAATTGTTTTCTTAATTTTT
TTTCAGATTTTCCATTGTTAATATATAGAAATTTAACTTTTTAATCAAGAAATACATTTTTTTTTTAGTTTTGATCCTCAACTGTAACCCTGCT
TAATTTGTTTGTTAGCTTTGAGAGGGTTTTGGTGAATTGTGTAGAATTTTCTACATATAAATTGATGTCATCTGTGGATGGAAACTGCCTCCTT
TCTTCCTATTTGAATGTCTTTTATTTCTTTTTCTTTCCTGATTGCTCTGGCTAGGACTTCCAGTGCTATGTTGAACAGAAATGGTAAAAGTAGG
CATCCTTCCCTCGCTCCTGATCTTAGGGTGAAAACTTTGAGCTGCCTGCATATATGGTGTTGGCTGCAGGTTTTTCTTTCTTTCTTTTTATTTA
TTTATTTTTAATTTCTATTTCAATAACTTTTGGGATACAAGTGTTTTTTTGTTACATGGACAAATTGTTAGTGGTGAACTGAGATTTTAGTGCA
CCCCTCACCTAAATAATGTACATTGTAACCAATATGTAGTGTTTTATCCCTGGCTTCCCTCCCAACCTCCCTCTTCTGAGTCTCCAGTGTCCAT
GATATCACTCTGTATGCCTTTGTGTTCTCATAGCTTAGCTCCCACTTATAAGTGAGAACATAGGATATTGGTTTTCCACTCCTGAGTTACTTCA
GTTAGAATAATGGCTTCCAGATCCATCCAAATTGCTGCAAATGACATTATGTAAATCCTGTTAATGTCTGAGTAGTATTCCATGGTGTATATAT
ACCACATTTTCTTTATTTTATTTTATTGAGATAGGGTCTCACTCTGTCATCCAGCCTGGAGTGCAGCGGTGCGATCTCGGCTCACTGCAGCCTC
CACCTCCTGGTTTCAAGCGATTCTCCTGCCTCAGCCTCCCGAGTAGCTGGGACTACAGGCACCAGCCACCAAGCCCCACTAATTTTTGTTTTTT
CAGTACAGACGGGGTTTCACCACTTTGGCTGGGCTGGTCCTGACCTCCTGACCTCAGGTGATCCACCCGCCTCAGCCTCCCTAAGTGCTGGGAT
TACAGGCGTGAGCCACGGTGCCTGGCCTTATACACCACGTTTTCTTTATCCACTTGTGAGTTGATGGGCACTTAGGCTGGTTCCACATGTTTGC
AATTGGGAATTGTGCTGCTGTGAACATATGTGTGCAAGTGTCTTCTTCATATAATGACTTCTTTTCCTTTGGGTAGATACCCAGTAGTGGGATT
GCTAGATTGAATGATAGAACTACTTTCAGCTCTTTAAGGAATCACCATACTGTTTTCCACAGTGGTTGAACTAATTGACATTCCTACCAGCAGT
GTATAAGCATCCTCTTTTCCCCACATCCATACCAACATCTATTATTTTTTGATTTTTCAATAATGGCATCCTTGCAAGAGTAAGGTGGTATCTC
GTTGTGGTTTTAATTTGTGTTTCCCTCATGATTAGTGATGTTGAGCATTTTTACCTATGTTTTTTGGCCATTTGTACATCTTATTTTGAGAATG
TCCATTCATGTCCCTTACCCACTTTTTAATGGGATTATTTGTTTTTTCTTGCTGATTTGTTTGAGTTCCTTGTCAATTCTGGATATTCGCCTTT
GTCAAATGTATGCTTTGTGAATATTTTCTCCCACTCTGTGGGTTGTCTGTTTACTCTGATGATTATTTCTTTCTGTGTGCAGAAGCTTTTTAAT
TCAATTAGGTCCCATTTATTTATTTTTGTTTTTGTTGCATTTGTTTTGGGGCTGTTAGTCATGAATTATTTGCCTAGGTTGATGTCTAGAAGAG
TTTTTCCAATGTTGTCTTCTAGAATTTTTATAGTTTCAGGTCTTAGATTTAAGGCTTTGATCCATCTGGAGTTGATTTTTGTATAAGGTGAGAG
ATGAGCATCCAGTTTCATTCACCTACATGTGGTTTGCCAGTTTCCCCAGCACCAATTATTAAGTAGGGTGACCTTTTTCCAATTTATGTTTTTG
TTTTCTTTGTCAAAGATCAGTTGTCTGTACATTTTTGGCTTTATTTCTGGGTTCTCTATTCTGTTCCATTGGTCTATGTGCCTACTTTCATACA
AGTTCCATGCTGTTTTGGTCACTATAGCCTTGTAGTATAATTTGAAGTTTGCTTCAAATTAGTATAGTAGTATAATTAGTAGTATAATTTGTTT
TTGCTTAGGATTGCTTTGGCTATTTGGGATCTTTTTTGGTTACATATAATTTTTTTTTTTTTTTTTTTTTGATGGAGTCTCACTCTGTTGCCTA
GGCTGGAGTACAGTGGAGTGATCTCGGCTCACTGCAACCTCTGCCTCCCAGGTTCAAGCCATCTCCTGGATTCCCTGCCTCAGCCTCCCAAGTA
ACTGGGATTACAGGCATGTGCCACAATACCTGGCTAATTTTTTTTGTTGTTGTTTTTGTTTTTTGTATTTTTAATAGAGACGGAGTTTTGCCAT
TTTAGTCAGGCTGGTCTCAAACTCCTGACCTCAAGTGATCCACCCACCTTGGCCTCTCAAAGTGCTGGGATTACAGGCTTAAGCAACCACACCT
GGCTAGTTCCGTATAAATTTTAGGATTGTTTTTCCTAATTCTGTGAAAAATGATGTTGGCATTTTGAGGGGAATTGCCTTGAAGCTGTAGATTG
CTTTGGGCAGTATTGTCATGTTCACAATATTGATTTTTACTGTCCATGAGCATGGGATGTGTTTCTGTTTGTTTGTGTAGTCTATGATTTTCTT
CAGCGGGGTTGGCTGCAGGTTTTTCACAAATGCCCTTTATCCAGTTGCCCCAGGTGGCAGTGGCTTTATTTAACTTTCAAGGTTTTCAAAGACC
ACCCTTCAATTAGCCACTGCTCTCTCCTGAGACAGTTCTGAGTTAGGCAAAACGAGTTTTTTTTTTACCCAGACAGGTTAAAACAGACAAACAA
TTCCTTGGGGAAAAGGTCTGCTCCGCTCCCTCCAGAACTATCCACCCACCTGGGGAACGCCGGCCTTCACTGTCCCAGGCAGGTTTGTAGGCAC
CACCACATGAAAACACTACCAGGCTCTCCTAGCAAGTCAAGGTCATCATTTCCTCAAGTTGGCATTCAGTGGGTTGCGGTGACCCTTTGACTAC
TTTCCAGAGAGCTAACAATGTCGGTACTAACCGTTTGTGTCTAATTTTTTGGAGGGCCCTTGGAGCTTCCTTTTGGGTTCCAGGCTGCGTTCTG
GGGTAGAGGACACAGATCCCACCACTCGATGGAGCATCAAGGTCACCTTGGGAGACCCACATGTAGGGCGGAATGTGCATAGTACCATCCACCC
CAGGGTGGCTCAGCACAGTCCTGCCGGCAGAGGCGGGGCCCAGGGAACCACAGCTGTGCTGAGCATGTGGTCAGCAGGCCGAGGAGACTTTGGA
GACCATGGTGCCGAAGGAAGGCTACTCAGACAAAGCGAGACAGGTCACGCTGGGAAAGTCCTCCCAGGGACCTTGCAGTCCTGGGGGTCCTGTT
GGCAGAATGCCCAAGGCCAGAGTTCATCTCACTTCTCCACAAATCTGCTTCCCCGCTGTCTCCCTCATTTTATAACGGACAACCCCAGTTCTCT
GTTGCTTAGGTCAAAAACCCTGGGCCCCTGGCTGCCACCTTACCCTCCTCCCTGTCCTCCCCGCAATAGTATGAATGCTTGTGCCCACCCCCCC
CCCAATTCCTACATTGAAATCCTCACCCACAGGTGACGGTGTTAGAAGGTGGGGCCTTTGGGAGGTGGTCAGGTCATGAGGATAAAGCCTCATG
AGTGGGATCAGTGCCACTGAGGCTGAGGTGGGAGGATTGCTTGAGGCCAGGAGTTCAAGACCAGCCTGGGAAACATAGCAAGACCCCATCTCTG
CAAAAAATAAGTTAACATATATTAGCCAAGCACAGTGCCTTGTAGTCCTAGCTACTCAGGAAGTGTCACTTGAGTCCAGGTTGAGGCTACAATG
AGCCATGATGGCACCACTGAGCTCCAGCTTGGGCAACAGAATGAGACCCTGTCTCTAAAAAAATATAAAAATAAAAATAAATAGGCCAGGCGTG
GTGGCTCACGCCTGAAATCCCAGCACTTTGGGAGGCGGAGGCGGGCAGATCACTTGAGGTCAGGAGTTCGAGACCAGCCTGGCTAACATGGTGA
AACCCTGCCTCTACTAAAATACAAAAAAATTAGCCAAGGGGGGTGGTGGGCATCTGTAATCCCAGCTAGTCGGGAGGCTGAGGCACGAGAATCA
CTTGAACCTGGGAGGCGGAGGTTTTAGTGAGCCGAGATCGCTTCACTGTACTCCAGCCTGGGCAACAGAGTGAGACTCCATCTCAAAAAAAAAA
AAAGGAAAAATATAAATAAATAAACAAATAATAAATAAATAAAAAGGAACCTCTCAGTCCTTCCACCATGCATATGCTGACCCAGCGAGAAAAA
GAGGTCTGACCCAGTGAGAAAGAGAGGTCTATGAACCAGGATGTGTCCTCGCCGGGCACTGAACCTGCCAGTGCCTTGCCCTTGGACTTGTGCT
GTTTATGAGCTGCCTGGCTATGGTGTTTTTTTAGAGCAGCCCACACGGACTGAGACACTCTCTGTGTCTTCCAGCCCCACTGGCCTCTTGGCTG
CTTCTCAAACACTGAGCATGATGCCCCCTCCCTCCCACCTTTGCACTTTGCAGTCCCTCTGCCAGGAACACCCTTTCTCCTGTAGTAATTCCCA
CAGGCCCTCTCAGGAGTGTCCCCCACGCTTGCTCCTATCCCCCAACCCTGCTTCACAGCCTCTGGTCAGCCCCATGAGCTGATGATTCTGTGGA
AGGACAGCATGGCCTTGCTCAGCCCCTGCAGCCAAATGCGAAGGAAGACCACAAGTCCTTTGGAGGGGAGGTGCCTGGCCCTGGGGGGCCTGGC
ATGTGCACTGACCAGGAGAGCTCCTGAGTGTGCAGCTGACACTGCCTCCAGGGCTGGTGGGGCCTGGCTGAGGCCATCCATGGGCAGGAGCCCT
CCAGCATCCAGTGGCCATGGCCACGCCCCAGCCCTCATCACGGGCCTGTGTTCTTCAGGCGTGGGGAAGGCACCGTGGGGCAGAGAGTGGGGAC
CCGAGGGTCAGGCTCAGAGTCCGGGCACTGGTCACTGTGGGTCTGGGATGAGGAGGACGTCCCTGAAGGACAAGTCAGGGTCCCTGTAGGAGAA
GAAACTGGAGCTTGGGGAGCTTGGATGGTGGCCTTCCTGTGATCTTGAGTGAGAGGCAGCTCACTTCCTGGAGCATCAGCCTCCCATTTATAAA
GTGGAAGAATACTTACAGCTTGTCAGACCTCACAGAGCTGCTGGCTGGGGTAATACACCACGCAGCTCCCCTGGAGATAGCTGCCAGGGTGCAC
AGGAGTCAGGTCCCAGGGCCGTGCAGCTCATCGAGCGTGTCCAAGGGTGTGTGTGGCGTGTGATGCATTGTGTGTATGTGAGTGTGCACGTGTG
TGGGAATGTGGTGTGATGTGTCATGTGTGCACATGCATGTGGTGTGTGGGGTGTGTGCCTGTGCATGTGTGTGCATGTGTATGTGTCAGGTGTA
GTGAATGTGTATGTGGTTTGGTGGATACATGTGCCTGTGCATGGGTGTATGTGGATGTGCCTGTGCCGTGTGCATGTGGCTGTGGCATGCATGT
GCATGCCTATGTGTGCATGTGATGCAGTTGCTGCCCGCTAGGGTTCCTGGGCTGTGCTGTAGCCAGTGGTGGCATGGAGGGAGGCATGGGCAAC
ATATACACTGCAAAAGGCCAAGAGCACCAGACTGGGCCCTGTGCCATGTGCTTGCTGGCTCCAGAGCCAGGATGGGGTGCTTAGGGCTGCCACT
CCCCCTCTGAGAAGGAAGCAGCGATTTTAGGAAGAAAGCAGGAAGCCTGTCCTGCTTGGAGCCATACCAGGTGATGGAGGAAGGCTGTGCTGCA
TGGGGTCAGAGGGGAACATGCCATTCCCTAAAGCCCTGCTACCCTCGGGAATCTTTAGGATATTTCATTGCCCTCCAGAATGTCAGGGTGTCTC
TCACTTGTGGAAGCAGGGAGGACATCTCACTTGCCAAAGCGGGGAGGGCTCCTGCCCATGGACGGCATCAGCTCACTGACTCCCAACCTGGGCA
TCTGAGCCCAGCTGGAGGCCAGGAGGAAGCTAGCCTCATGAGTCACACCATTCAGGAACCCTTGGGTCTGTCCTCCAAGCTGAGCCTTGGGGAG
CCTGTGCATCACTGTCTGGATTGGACGATGCCCATGGAAAGGCACAGATCTGCACAGATGGGAATCCCAGCCAGCAGATTGGCTGGGGGCCCAG
CCAGCTGAATGCAAAGCCCCAAAGTCGGTGAATAGCACTGGGTCCTAAACCTGATGGCAACTCCAGCTGCTTCAAGGAAAGGCCTATGAACGAG
TCCTGCTCTGGAGTCCCCAAATCTATGGTCTAGGAGCCTGGTCATATACCAGAACTTCCTTCCCTTTTATGGCTGAGTAATATTCCATTGTATG
GATAGACCATATTTTGTTTATCCATTCACTCACTGATAGGCATTTGGGTTTTTTCAATTTCTTGACTATTATAAATAATGCCTTTTGTGTGAAT
GTGTATTTTCAGTTCTCTCTGGCATATACCTAGGAGCAGAATTGGGTCACAGAGGAACTCCATGCTTAACTTTTTGAGGAACTGTCAAACTATT
CCAGAGTGTCTGCACCATTTTACATTCCCATCAGCAGGATCTGAGGATTCTGATTTCTGCACATCTTCATGAGCACTTGTGATTTTCTGCTTGT
TGTTTTTGTTCTAGCCATCCTAGTGGGTGTGAAGCAATGTCTCACTGAGGTTTTCATTTGCATTTCCCTGATATCTAAGGATGTTGAGCATCTT
TTCATGTGCTCATTGGCCATTTGCCTCTCTTCCTTGGAGTAATGTTATTCCATTCTTCCACCCACATTTTAATTGGGTTATTTGTCTTTTTGTC
GTTGAGTTGTAAGAGTGCTTTATATAGTCTGCATACAAGTCCCTTATCCAATACATGACTTGCAAATATTTTCCCCCATTCTGTGGGTTGTCTT
TTCACTTGCTTGATGGTGTCCTTTGATACATGCAAGATTTTAATTTTGACGAATTCCAAGTTATCAGTTTTTTATTTGGTTCCTTGGCTTTTGA
TGCCATATCTAAGAGACAATTGCCTAACCCAAGCTCACAAAGATCTACTCTTCATTAGGTTTTTTTTAAATAAGAGTTTTAGCGTTTTAGTGCT
TACATCTCGGTCTTTAATTTTCATCGACGATGTGAGGCAGGGATCCAATCACATTCTTTTGCATGTGGAAATCCAGTTATTCCAGCACAATCTG
TTGCAGACTGTTCTTTCCCCTTTGAATAGTCTTGGCAACTTTACTGCAAATCAGTTAGCCCTAGATGTATGAATTTATCTCTGTGCTCTTTATT
CTATTCCATTGTTCCATATGTCTGTCTTTATGCCAGTGCCACACTGTTTTGATTATTGTAGTTTTGTAGTGTTTTGAAATTAGAACATGTGAGT
CCTCCTGCTCGTCTTTCTTTTCAAGGTTATTTTGGCTATTTGGAGACAATTACAGTTCCATGTGAGTTTTAGGATTAGCTCATCAATTTCTGCA
AAAAAAGCTTAGATTTTTAGAAATTGTGTTGAATGTGTAGATCGGTTTGGGAAATATTGTTCTACTAATATTATGTCTTCTGGTCCATGAACAT
AGGATATCTTCCTATTTAGGTCTTTAATTTCTTTCATGACATTTTGTAGTTTTCAGCATACAAATCTTGCACTTATTTTGTTAAATGTATTCCT
TAAGTATTTTATTATTTTTGATGCTGTTGTGAATGAAATTGTTTTATTAATCTCATTTTTTAATTATTTGTCACTAGTGTATAGAAACACAAAT
GAGGCCAGGTGCAGTGGCTTATGCCTGTAATCCCAGCACTTTGGGAGGCCAAGACAGGGAGATCATTTGAGGTCAGGGGTTCAAGAACACCCTG
GCCAACATGACAAAACCCTGTCTCTACTAAAAATACAAAAATTAGCCAGGCATGGTGGCACATGCCTGTAGTCCCAGCTACTCAGGAAGCTGAG
GAACAAGAATCACTTAAGCCCAGGAGGCGGAGGTTGCAATGAGCTGAGATCACACCACTACACTCCAGCCTGGGCAACAGAGTGAGACTCTGTC
TCAAAAAAACACACACACATACGCACACACACATACACACACAAATGATTTTTAATATAGATCTTGTACTCTGTAACCTTGCTGGAGTTGTTGA
TCAGTTCTAATGTATTTTTTAATTTGCAGATTCATTAGGGCTTTCTACGTATAAGATAGTTTTATTTCTTCCTTCCAATCTGGATGCAGTTTTT
TCATTTTTCTTCCTTAATTGCCCTGGCCAGAGCCTCCAGTGCCATGATGAATAGCAGAGTTGAGAGTGGACAAACAGCTTTCAGTCTTTCACCA
CTGGATAGGATGTTAGCTGCAGGTAGGCCCCTCTTGCTTTTAAACTTAGTAGTTCTAGATGTGAGAAAAACTTTGTTCACATAAATAAATAGAT
AAAAATGGGAGCACTTAGTTACCATGCATCTCTCAATTCTTTGAATTTTTTTAGAGTTATATATGCCAAAAATCACATGGTGGCCTATCATCAA
AATGTGAAAAAATCCTATCAGCCCATGACTCAGTTAGGTCCTCCTTAAAGTTTGTGGAAAACAGGGAATTAAAGGTGCCTGATTTTCACAAGTC
CCAGTCATTATCAATGGCTCCCACTCTTGCTTTCTGGGAGAAACTCCCTAGGGTATGGCACACTCCAGGAAGATCCAGGAGTCTCCTGGAACAA
GAGCTGGGACCCTTGGGGGTCTCCCATGGGTCTGGTGTGTGACAGCATCCCTATAGCATATGCCACACAGGTATACATGTGGCGCAAATGTACA
CAGTCGGTCCCCTGGGAAAGCACAGAGATGCACTCTGCCACACAGCATATGCTTAGAAATGATGTATTAAGCCAGTGACCTAAGGACAATGGTG
CATGACTCGGGGAATCCCAGGAGGGCTTCCCAGGGGAGGTGGCCCCTGGGCTGAATCTTGAAGGATTTTCTTCCTTTAAAACACACACATATGC
AGTATTTTCTAGGCACCATTTAAGTGTTCGACACACCATGGCCAGTTGAATCCTCATGCCCATCTGTTGAGGCCAGCAGTGCGGTGACCCTGTT
TTACAAATGAGGAAGCAGAATCACTGTGACAAAGCAGAGACATGAACCTTGGCAGCCTGGCTCCAAAGTCCCCAGGCAGAGGGGACAGCCCATG
CCAAGGTTGGGGCTGACGTGGGGGTGAGCAGAGCCCTCAGGCAGGCCCAGCAGTGACAGAGCCTAGCTGGAGCATACAGTTTAGGAATCACTGG
AGGAACCCCAAGCCCACAGGGAGGGCAGGTGCAGCCCGGGAGGAAGCAGAGAAGGTAGGACTCCAGAGGATACCCGGTCCCCATGTGTGCATCC
CTCCGGGCAGGGCGGGGATGGATCTGAGCGGTCACTGAGAATTTCTCAATGGGGCTCTTGGATTAATGCCGCAGGCTCTCCAGGGGCAAGTGGG
CCTCACAGATGCCCAGGTCCTGCCCTAAGGTGCCAGCGTTGGGTTGGGGCGGGGAGCCCCCATTTCCGGTTGTTTTCCTGCTGCTATGCCGGCC
TCTCTGCAAAGGGCTGAGCTCCTGCTGTGCTTCTAGCCTCCCCTCCCATCCCACTTACCTCCCCTGGACCCTCTCCTTCTCCGCTTCTCTGATC
CTACTACCCCCTTCTCCTCCCCTTTCTGATAGAGGAACACAGTCTGGAACCCCTCCTACCTCCTCATGGCTTAAATAGGTTTAAGTAGGAAAGG
GGAAGTCCCGGCTCCTCCCTCCTGCCTGCCATGGGGACTCAGAAATGCTTGGAGTCCTGGTGGCCTGGGAGGAAGGAGCCCGCCGGGTGCCTCA
GAGAGAGGCCTGCTCAGGAGGCCATTGTGGAGGCCCTGGCTGCGCTCTCCATAATCATATGATTGGATTAGGGTCAGGGCAACCCTAATCATGT
CAAAGGTCACCCCAGGCTTTGTGGCAGTGGCCACAGGGTGCCTGACCACCCCTGCCAAGATGGCCTTGTTGAACACAGTTAAAAACCCTCAGTC
AGCTGTGAAGAGAGGGGCCCAGCCTCTTGTGTGTGTGTGCACATGTGAGTGTACACCCTAGCGTGTGCATTGTGAGGGGTGCATGTGTGTGCCT
ATATGTGCACATAAGCCATATATGTGCATGCCTGTGTGTATGCATGTGCATGTGTGTGTGTGTGTGTGCATGAGTGTGAACATATGAAGGAAGA
AGCTTCATATCCTCACACCTCCTGCTTTCATCCCCATCTGCCGTCCACCTGGCCTGGGGCTGGGGACTGGGGCCACCAGACTGAGGCTGGGAGA
AACCATCAGTCCTTGGAACATGGGGCTCTGTCACAGAAGCTGAGCCTGGTCCCTGAAGAGCCCTCTCTTCTGGGGTCCAAAAGGAGCAGGACAT
TCAACCTGCCTGGGGTAGAAAGCATTTGAGGAATTTTAAGTGCATGGAGACTGGGCACACCGTGCAGCTTCTCTGTCCCCTGCCGCAGGACCCA
GGCACTGGGAGGGCTCACTTCCCTGCAGAGCTGGAGACACCTCACTCTTGACCCCTGGCAAGGTAAAGCTGGCAAGGACACAGGTACAGAGAAA
GGGCAGGGACGGCTGCAGTGGATGCAGCAGCCCAGGGCCCAGCAGCTGGATCTCAGGCAGCACGTGGCTCTGCAGAGAGCCTGGCCTGCTCTAG
GCCAGGTGGGGCATGTGCTCCTTAGAGAAGGGGACAGAGGCCAGGAGCGGGCGAGGCCTGGAGCCTAGGGATGCCACCTGTGCCCACATGCTCG
GTGCCCTCTAACATGGGCTGGGCCCAGGAGTCCCCCCGGGCCCTGGCACCTGCCAGATAGGTCGGGGGGAGTGGAGTGGCATGGGGAGGTATGC
AATCTCTTGTGCTTCCCAAGTAAGTGTGCTTCCCACAACTCCCAGTGGACACTTCCCAGAGCCCTGGGGTTTGCTTCTTCGTACCCAGTGTGGG
TGGGGCCTGCAGCAGTGGCCCTCCCACTCAGCCTTCACACACCACCCCCTCTTCAAGCATTTCCAGTCCCCACCCCCTGCCCTATTCTGTGAAC
CCCAGGGTATCCCCACTCAAAGCACACGGCCAGATTCCTGGTTGGTGCCCCAGGACCCCTCCAGCCCCAAAGGGTGGGGAGAGTAATGAAAGGG
TCCTGCGCACCACATGTCAGTAAGCCTTGTTGGGTGCCCCACAAGTCCCCCTTCATCTCTCCAACGTCTTCCCCAGGCAGGAGACAGCCCAGCC
TCCTCTCCTCCCCGCTGAGCCCCCAGGAGCCCCTCGGGGGTGGTCCGCCACCTCCCTGTGTGTCCCCTGCCTATGAGGGCCGGCACAGGGGTGG
GGCCTGCCTGCTAGGACATGGGGACACACAGGGCAGCTCTTTAGGGAACCAGCGTCCCCAAAGCCAGGTTGCGACCCCTGACCCCCCCCATCGG
AAAGTGTTGCCGTTTAAGATGCGTTGGGGTGGGGGGCTCCCCTGGCTCCAGCGGCCTCTCTGGTATATCATTAACTCTCCCCAGTGGGAGGGGA
ACCCCGGCCGACTCCCCGCCGCGTCCGGCTGGGAGCACAATATCCCAGCGACGGCGACAAGATTAAGCACAAGGGGAGAGTGCAAATGCGCGGG
CGGTTCACACGCCCTTCGCGGTGCTGAATTAAAGCGTCTGAATGGGGATTTCCCCTCGCGCCGGACGGCCTGGCGCGCGGCGCATACTTCCAGG
TTGCTATGACTGGCCTGCGCTCGCCTAATCACCGACTAGGGGGAAATTCATATAAAATATCGCCGGCCATTGTGGGCCTAACTCGGCGTGACAG
TGGCGCACAAAGCCGGATTCAAGCGCCCCAGCGGGTCAGCACGGCGCCCGGCCGCGCGGGCCGCCCGGCGGTGTCAGGGGCCGTGGCCGCGGTG
GGGCGCGGGCCGCATTCCGCTCTCAGGTGGCTCCGGGGCCTTTTGTGCAGGCCATGATTACCAAATGCCACCGCGACACCCCCTCCCGGCCGCC
TGGCTCCTCCTGGGGCCGCAGGAATGAGGGAAGGTGGGCAGCCCCCGAGGGGAGGCGCCCGGGCGTCGGCTCCGGCGGGCTCCGGCGGGGACCG
GGGCGCGGGCTGAGTCCCCCTGTGTGCCCCACAGCAAAGGGTCTTCTCCTTCCCCTGTGTTCAGGCCTCAGTGGGGGAAAAGGGCCCACGCGTT
GGCTGCTCTGGAAGCCTGTTGTGGCCTCCCGATGGGCACGAGAAGGACTTTGCTTGTCCTTTTTCAGGGGCGATGGTCCCCAGATGACATGACA
TTTGGGGCCACAGTGACCTGCAGGCCCCTGGCCAGTCTGGTCAGGTGGTCCTAGCTGGGATCCAAGGCACCCCCCTCACTCAGCCAGCCCCTTC
CAGCAGCTGTGGCGTGAGCAGGCAGGAGATGGGGGCAGCACCAGGAGGGAGGGACCCAATGCATTCGGGGTGGCCACTGTCCCCACAACTCTAT
GGGTCCCAGGTATGTGGTGGGGGTGTGGTAATCATACAGGGTGCAGCGTGCACGGGGGGCTGTGTTCGCTGTGACCCTCCATCCCCCAGGATGC
CTGAAGGCCAGTCCCTGGAGTACAGGGAGACTGCTCCTCACTGGTGCATGGAAAGGGCTGGCAGATCAGGCAGGGGGAAGGCCTGTGGGGTGGG
GCATTTGCCCTGCAGACCTGGGGAGAGAAGCTGGCTGCAGTAGGGTGCACTTGGGGGACAGAGGGCATTTTTTTGTCACAGTGGAGGTGAGAAC
TTCCCGGTGGAGATTCAGCAGAGTTTTTCTCCTGGGCAGTGGGCAATGTTGGGAGTCTGGGCCCCCAGCACCCGGCTGGACTGGCCACCATGGA
GTCCTGCCCTCGGCCAGTGCATCCCTGCCAGAGCTGGGGGTGTCCAGCCAGACAGTCAGCATTGCCAGGGGAAAGAGACGAAGCCACAGGTTTC
CAGGGGCTGGGGACAGCATTGGAGGGCCCCAGGAGGCATGGCCAGGTGAGCATGGGTCAACATCAAGAAGCAGACTGGGCAAAGGCGGCATCCC
AAGCTCCTAGGAAAGGAGGTGTCAGCTGCCCCAGGATGGGGCCACAGAGGCAGAACCATAGCCCCCTTCCCAGACTAAGCAGCCCAGCCACCAG
CTGCCAGCAGCCAGGGCCCTGAATCCCAGAGCCTGTGACCAAGGTACCCACCTCCAGGAAGAGGCCAGGCCAGAGACCCAATGACCACTGTCTA
CTCATGGGGGGATGGCCATCAGCATATCAGAGACACCCTCCCAACCCCCTTCTCCTGGCAGGCTCCTGTTCAGGGTCTTGTCCAGGGTCCCATA
GAGCCCTTACAAAGTCATGGCCAAGCTGTCCATAGCCTTCTTTCTGGTCACAGCCCAGAGGAGGGGTCAGCACGGAATGGAATCTGAGCTTGGG
AGATCTCATCTCAGTTGATGCATCAGAATCACTGGAGACCCTTTAAGATATAGGTGCCCCCCACCCCAGTTGAACTTGGGTCCCCAGGGCGGCC
CTGCAGTGATTCATTGGCCCCCCAGCCATGACCATTGTTGGAAGCCTCCAGTCCTAATTCCCTGATGGGCTTCCCAGAGGGGCCAGCCTGGGTG
AAGCCCTTCAGACTGAGCACTCTGCCTCCCCGAGGAGCTGGATTTCATTGTCCTATGGTCGCATTTAACAACAATCCCCACCCCCTGACCCAGA
ACTTTCCCTCCTTCAAGTTGTTTGTCCCTCAGAAAAGCCACTCAGGAGTCCCCTCTGGACCAACAGGCATGTCCCAGGAGCCCAGGAGTCCCAG
GCTGTCCCTCTCCTCTCGCCCTGTATCAGCCCCCACAGCCTTTCCACAGATAGGCATGTCCCCCTCAGCCATGCAGTGGGGGCCCAGCCAGGCA
GGTGGGCTCCCTGCTCCCTTGAGGCTATGGCCATGGAAACGCCAGCCATGGGAACACCCATTGGAGCTGCAGGAGCCCATAATGATGAGTTCAG
GGACCTCTCTTGGGACCCCTCTGTCTGGGCTCTGCACTCACTGCTGTGCAGCCTGGGGCCCATGGCTGACCCTCTCTGAGCCATCTTCCTGTTG
GGGCCACTTCTCTCATTAGTTAATACAAGGATCGGCTTGGACCTGCTTTTCCCACTGTCACCCCCTAACAGGGCAGCCTCCATGCTATGCTGCC
TCAGCAGCTACCTTCTGAGCAAACACCTGTCCCTGCCGAAATGCTTCTTGGGTCACCTCCCCATGGTAAGGTCAGCTCTGAGAGGACAGAGGCT
GCAGCCTTCTTTGCCACTTAAAAGACAGTCACTGCGTAATAAATAGTGGCAACTGAGCAACTTTAGTGAAATGTGAAAGACAAGGATTTTGGAA
TGATTTGGGCAAATGTTGTTCAACGTTTAAGAATTTGGAAACATGGCCGGGCGCAGTGGCTCATGCCTGTAATCTCAGCAGTTTGGGAGGCCGA
GATGGGCGGATCACGAGGTCAGGAGATCGAGACCATCCTGGCTAACACGGTGAAACCCTTTCTCTACTAAAAATACAAAAAAAAATTACCTGGG
CATGGTGGTGGGCACCTGTAGTCCCAGCTACTCGGGAGGCTGAGGCAGGAGAATGGCGTGAACCTGGGACGTGGAGCTTGCAGTGAGCCGAGAT
CGCGCCACTGCACTCCAGCCTGGGCGACAGAACGAGACTCTATCTCAAAAAAAAAAAAAAAAGAATTTGGAAACATATCCTTCCTGTTTGATGT
TCAAAGGTCCTTTTCTGAAGTTATGATGGTAGATATTTTATATATATATATATATATATATAATATCTACTACATATCTATATCTATCTATCTA
TCTATCTATCTATCTATCATCTATCTATCATCTATCTATATCTTTTCCTGTCTCAACAAAATAAAAGCACTTGGCAGTCCTCTGTTAGTCCCCA
GATGCTCTCTGATACTTGAAAAGCCCATTAAATCCAAACCTAGAAACGAAGGATTTGTTAAGATCCCTCTAGTCCAAAAGTTCTGAGATGCAAG
AGACAAAGGAACCATCAAAGCTCATCTCATGATTCCCTGCATCTGACAACTGCACTGCCCTCAGCTGTGATGGAGCCTTGCCTGGCAGCTCTTC
TGCCTTTCCCTAAATGCCCACCTCTTACCACCTAGAACTCCCTCACCTTCTCCGACTTTATCCATCCATCCATCATCCATTCATCCATCATGCA
CGATCCACCATCCATCAATTCATCCATCCATCCATCCACCATCCAACATCCATCAATCTATCCATCTGTCAATCTGCCATCCATCAATCCATCA
TCCATCTATCCATCAATTCATCCATCCATCCATCCATCCATCCATCCATCCACCATCCATCAATTCATCATCATCCATCTATCCACCATCCCTC
AATCCATCATCTATCCACCCACCCATCCATCTACCCATTATTCATCACCCTCCCATCCCAGGCCTGCCTCAGAGCTCATGTTGTCAAGCCTTGC
TATGTGTCCTTGTCCTCTCTACTGCCTCCCTTGTGAACTGTAAGCTCCCAAAAGCACATGGTTTAGCACAGTCCCAGGCAAAAAACCTAACACA
CACACCAACAAAGCCTCCCACCAGAGATAGGGAAGGCTTCCCCTGGTTCATTCTCCTTACCAAGGCAAGGACTCTCTTCTCCTGAAAGTGGGTC
ATCCAACCTCTGTTTTATTATCACCAGGACAGGAACTCCTTCAACTTTCAGAAAGCCTAGGTTGTTAGAAATTGTATCATATTGAGCAGCAGTC
CACTTTCTGGAGGATTTTTTTTTCTTTTGATGGAGTCTCCCTCTGTTGCCAGGCTGGAGTGCAGTGGTGTGACCTCGGCTCACTGCAACCTCCG
CCTCCCGGGTTCAAGCGATTCTCCCACCTCAGCCTCTTGAGTAGCTGGCACTACAGGTGCACGCCACCATGCCCCGCTGATTTTTGTATTTTTA
GTAGAGACGGGGTTTCACCATGTTGGCCAGGATGGTCTCAATCTCTTGACCTCGTCATCTGCCCACCTCAGCCTCCCAAAGTGCTGGGATTACA
GCTGTGAGCCACCGCGCCCAGCCTCTGGAGGATTTTTTTTACTGTGGTTATTGAGAGTGGGCACCTTGCTTCTTATTCATCTCATATTCCTCAT
TCAGCCCAGTGCCTGGCTCATTAGGGACTCTCAGATCCTGGATGGATGGATGGATGATGGATGGATGGTGGGTGGTGGATAGATGGATGAATGG
ATGATGGGTGGATGATGGATGAATGGTGATGGATGAATGGTGATGGATGGATTAGAGATGGACAGATGATGGGCAACAGATAGGTGATGAATGT
ACAGATGATGAAGGGGATGATGGATGGATGATGGTTGAATGGTGGATGGGTGATGAATTGATGATGGATGCATGATGAATGGATAATGGGTGAA
TGGATGGATGAGTGGTGGTAGATGGTGGATGATGGGTAATGGATGGACAATGGATGGATTGTGGATGGATGGTGGATGATGGATGATAGATGTA
TGGATAATGTATGGATATGGATGATGAATGGATGAATGGTGGATGATGGATGATAGATGGATGATGGATAGATGTGGATGATGGATGGATGGAT
AATGGATGATGGATGGATGTGGATGACAGATGATGGGTGATGGGTGGATGGATAGATGGGTGGTGGATGGTGGATGATTGTACATGATGGATAG
TAGATATATGGATGATGGATGGATGGATGGTGAATGGATAGATGAATTTATAAGTGGATACATGGTAGGTGGTGGTAGAGGATGTATGATAGGT
AATGGATAGATGGATGGATGGATGGATGGATGGATGATGCACGATAGATGAATGGATAGATGGATAGATGTGGATGACGGATGGATGAATGAAC
GAAAGGAAGGAAAGATAGATAATTGATACTTTGGATGAATTCAGTAAATAACTGCTAAATGAGACCACAACAAGTTTCTGGGAAAGATGGTTTT
CCCAGGTGGAAGATACTTCCCTTACAGAACAATTTTGTTATGCAGTTTCCTTTCTTCTTCCTTTCTCCAACAACTGATTGCTGTGTTAGAATAA
AAGTCTTGGGCCTGAATGGGGCAGGCCTTAGGTGGCTCAAAAAGTTCAGCCAGGCTCCTTCCCAGGTGACTTGGCCAAATTGGCAGCATTTGTG
TCTAAGCACCCTGCCTCCCAGTAGCCTGTGTCCCATTCCAGTGGGAACTCCGACAGTCCACTCTAATGAAGGCATTTTCCTGCTGACTGGGATG
ATTTCCTGGCCTTCTGGGATGGCTGGCACTTCCCACGGGCAAGATGCCTCCGTTTCACCCCCATAGCTGACTCTGTTGTGTCAACAAAAGCAGA
AACCCCTGACAAAGGTGTTTGTGGTAGAGAACTTGGTTTCCGTGGAGGCTGGCTGCCAGAACTCCAGAGCCTGGACGTAAGTCCTGGCCTCAGG
GGAGCCCTGAGTCAGCTCCTCTGTAAACAGAGGTGACTCTGGGCAGTGGTTCATAGCCTGCATGCTTCAAACACCTGTGGAAATCTGATCAAAG
CAATGGATTCTCTCTCCAGAAAGATGCCCGTGCTCCCAGACATACCCCATCGTTTGTGCTATTTTGGGAGGGTAGTGAACCTGCTGCAGATTCC
CTCTAGAACCTTGGGTGGAATCCAACCCATAGGCTCAAATCTCCTGCCGCTGTGCCTGCCACTTGGACATTCTGGGGGGTCTTCCTTCTGCATG
GTTTCAGGACATTTTTTAACTTTTTATTTATTTTATTTTATTATTTTTAGATTAGAGATGAGGTCTCATTCTGCCTCCCAGGCTGGAGTACAAT
GGTGTGAACACGGCTCACTGCAGCCTTGAAGTCCTGGGCTCAAGCAATCCTCCTGCCTCAGCGTCCCAAGTAGCTAGGACTACAGATGCACACC
ACCATGCCCAGCTCATTTATGTTTGTTTGTTTTTTAAGAAAGGGGGTCTTGCTATGTTGCCCAGGTTTAACTTTTTATTTTGAAATAATTTCAG
ACTTTCATAAACATCGCAGGAGTGGCACTGAGAGTCCTGGGTCCCCTCGTCCAACCCAGTGGTGCATAACTATGCCAATTGTTCAAAACTAAAT
GTGTTCTATTCACCACACTCTGGACGTCACTCAGCTTTCCTTCGTTTTTCTATCAAGATCCCTTTTCTGTCCCAGAACCCCCTCCTGGGTCCCT
TGTTACTTTTGGTGATGCCATGCCTCTGGCTTCTCCACTCTTCCCTGTCCCTCATGCCCCTGAGACCTCTGAGGAGCACAATTAGGCATTTTGC
AGAACAGCCATCAATTTGGGTTGGTCTGATGTCTCCCGACAATTAGATGCAGGCTGTGCATTTTGGGTGAGAATACCTCAAAGGCTATATTTGG
CCTTCATGCATCATCCCTGGAGCCACAAGGTATTGTGTGTCCCATTTCAGGCAAGGTTAACTCAGTCCCCAGAATAAGGTGGTGTCTGCCAGGC
TTCTCCTGTGCACAGTTAAGGTTTTTCCCATTGTGCTTGATAAACACTGTGAGCAGTTGCGGAACCGCTTAGAGACCTTGTGGCTTCAACTTTA
ATGCAGGCTCTCTTTGCCAGAACATAGGGTCTCACGCTTTTCTGGCCTGTGGTCTTGCAGCCTGCAGCTTCTACTTCAGCCTCGGATGGGGGTG
TCTGTCCTGGGATCTGAGCCTCTCTGCAGAGACTTCTTGTCTTCAGTCTTCACGAGTGAATTGAAGGCCTGCTCCAGGGTTTTCTCAGCCTGGC
AGGGAGCTCCACCTCCACATGTGATGGAAGAGAAAATCAAGGTTCAGCCAGCTGGTGACCAAACTGAGCAAGAATGAGCTCTTTTCTGTCCAAG
TGGAAGCCCTCAGCACCACCCGCCCTTGCCCTTGAGTGTGTCTGGCTATTGGGGCTGTTTGCCTGAGTTTTGATAAGCAGGGATCTCGAGGCCC
TTGTTATGGGCAGGGCATGAGTGAGGCACAGAGAGCCCTAGCCTCAGGCTCTGGTTCTCCTCTTACCAGAAGCTCAGGAGCAAGAGGGAGGGGC
TGGCCTTGGAGACAGACTCTTGTAGTAGCCAGGCCGTGAGGTGGGAAGGCAGACTCAGCTGGGGTGGGGTATTCAGGGTGTGGGATGCCCAGGC
CATTTGGGAGTGAGAAGGTCCGGCCAGGAGGGCAGGTGGAGCTGTGTGCCATGCGTGAGCTCCAGGAGCACCCAGGACACTCCTCAACTCTACC
TCTCAAGCTGCCCTAGAAGGGAAGCAGGGCAGATGGCAGGGGAAGGGGCGCTGACTAGGGAGGGGACGACCTGAGTCCTTTAAAGACAAGAACA
GGCGCTCAGGCCTGGAGGCTTAGAGAGGAAGTGCTCTGCATCCAGCCACACGTGTATTCACTCAGAGGCAGTGGGTTCAGCAAAGGCCCCTCCA
GGCCTGTCTGTGAACTGATCAGTTGTGTTAGGGAAGTAACATGGGGTCAGGGGTGGGGGCTTTAAGTTTCTTGGAGAGGTGCGGAGGAGGTGGC
TGCCTAGCTGCTCCCTGGGAAAGGAAACAGCGGAGGTTGCATGATGGGCCTGGGTGCCTTAGAGGGGCAAGGGTGGGTCTGCTCTGGGTAGGCT
CAGACCCTGCTCATTCCTTGACTCAGCCCACGGCTCCTGCCTTTCATTCTGGCCCCAGGAAATATCTATGGGCTGGGCCTTGTGCTCCAGATTG
CAAATAAAAGGTGAGCCAAACAGTCCCTGCCCCTAGGCAGCCGCGGCTAGGGAGGGAGTCAGTGGGCAAATCTTCCTGGTTGGGGAGGGGCAGC
CAGGTGTAGAGGACACCCGATCCCTGGCCGCTCCCCTCAGAGCAGGCAGGGGCACAGCAGGTCCAGCCTGAGCCAAGGAAGGAACCGGAAAGAG
CCCGAGGGGAGGACAAAGGGCTGAGTGCGGCCTGAGGCCAGGGAGTGGCATTGAGGTCAGGAGAGGAGATAGATCCAGAGGGGGCCTGGCATGG
CCAAACTGGCATTGGGCGAAGACCTCAGCCTGCAGATAGTGTGTTGCGAGGGAGAGGTGTGCATGTGTGTGCATGCATGTGTTTGCATGTGTGT
GGTGCACATGTGGAGGGGTGTGTGTGTGTGTGCATCTGTGTGCATGCATGTGTTTGTGTGTGGTGGGTTGTGAATGTATATGTGCATCCGTGGG
AGGTGTGCATGTGTGTGGTGTGTATGGTGTGCATGCATGCATATGTATGTGTGTGCATGTGTCTGTGTGTGCATTGCACGAGGCGTGCATGTGG
GGGTTGTGTGTGCATGTATGTGTATGGTATTCATGTATGTGTATGTGTTGTGTGGTGTGTGTGTGTATTTGTGTGTGTGCATGTGCATGGGGTG
GGGTGTGTATATGTGGGGTATGTGCATGCATGTGTGTGCATGTGTATGGTATGTGTGGTATGCCTGCATGGTGTGCATGTGTGGGGGGGATGTG
CATGTATGTGAGTGCATGTGTATGCTGTGCTTGCATGCATGTGTATATGTGTTCATGTATGTAGTGGGGTGTGCATGTGTGCTTGTGTGTGGTG
GGGTGTGCATGTGTGTGCATGCGTGTGATGTGTGCATGTGTGTGGTGTGCATGCCTATATGTGTGTGTTTGCATGTGTGTGTGCATGTGCATCA
GGTGGGGTGTGCATGTGTGTGTGGTATGCATGTGTCTGTGTGCGCACGCATGTGCGCATGTGGCTGTGCATGCATGTGGTGGAAGGAGGAAGCT
GGAGGCTGGGGCCTCCTAGGGGCCTCTTCATGGCGAGGTGGGAGATGGACCCACAGATAAGAACCAGGAGGGGCAAATTGGCTGAGAGGCCCCA
GGGCCACCTGGGAAGGCCACAGCAGCCTGGGCGGCTGGGGCTGTTTCAGGACTGGACAGTCTCCAGCCAGCAAGCAGAGCCCTTCCTGAGCTGG
TGGTGGGAAGTGGATACAGAAGAAATGGACAGTTTTCTTCTGGGCTTGTGGCTGGATGCAGTGGGTGTCCAGGCCTGACTTGGACAGGGCCCTA
GGGAGGGACGGGGCTGCTGGGACCCGCCATCAGGGGGCTGTTGGCCAATCCACAGAAAAGTCAACCAAAGCAGGCCCGAGGGGCTCAGGGGGCT
TTGTCAACAGCCAGGGGGATGCCAGTGCAAAGCCATAGCCTCAAATACCTTCCACTTTGTATGTTCTTGTTATGGGAGCAACGTGCGTTCTTCT
GTGCTGGCTGTGTGTGGATGTGTGTTGGGCCGTGTGGGAGCGTCTGAGTGTGTGCACACTCACTCGTGCCTTTCTGTCCCAGCTGGCCTGGGCT
CAAGGGCATCACGAGCCCCAGGGGCTGCTGTCTCGGTCCTCTCCTAGGCAGCCTGCAACCACATGCCTGTGTCCCTGAGTTAGGCCATTCTTGC
ATTGCTATAAAGAAACACTTGAGGCTGGTAATTTATAAGAAAAGATGTTTAATTGGCTCAGGGTTCTGCAGGCTGCACAGGAAGTGTGGCTCTT
GCTTCTGCTTCTAGGGAGGCTAGGAAGCTTCCAATCATGGCGGAAGGCAAAGGGGGAGCAGGCATGTCATGTGGCGAGAGCAGGAGCGGGATAA
AGAGTGGGGGAAGAGGTGCCACACACTTTTAAACAACCAGATCTTGCAAGAACTCACTCATAATCTCGAGGACAGCACCAAGCCATGGGACCCA
CCCCCCATGACCCAGTCACCTCTACCAGGCCCCACCCCAGCCCTGGAGATCACAGTTCAACATGAGATCTAGGCAGGGTTAAATATCCAGACTA
CATCAGCCCCATCCACCCGAGGATGAAAGCTCCCTGGATGTCCAGGTGGCAGAGGAGGTCACAGCCCCCTATGATGACTCTGTTGTGACCCTAA
GGGGTCCATTGCAGGCCTCTGCCAAGGAGGAGCAGGGGACACTGTGGGACGTAGCCCTGTATCCCCGCCTTGTCCCATGGGAGGACCCTGGCTC
CAGGAGAACCTCAGGCCTCCTTCTATCCCCATAGAGAGGGCCCATCGGAGCTCCCTTCTCCCTCCTGCTCTCCCAGGGCCTCGAGGGGAGAGAG
AAGGTCAGAGCCAAGGAAAGGTGGAGGCTCAGGACTCTCCCACAACAGAAGCAGAGACATGGAGGAACCTGCCCCAGGGCACACCTAGCCCCCA
GAGGCCAAGCCGAGGCTCAGGGGCTCCAAAGCCCAGGTGTCTTCTTGCTAGGTCTGTAAGATCCCTGCCCAGCAGCAGGTCAGGCTCTTTCCTG
GTTGGCTCCCTGCTAGTTCCCCAGCTCAGACTGCAATGCTTGGCAAACAGCAGGCACTCTGTTCCTCACCTGTGTCAGAGTGTGGGTCCAAAGT
GAGTGCCTGTCTCTTTTTATTTTGTTTTTTTGAGACAGGTTCTCACTCTGTTGCCCAGGCTGGAGTGCAGTGGTGTAATCATACCTCACTGCAG
CCTTGAACTCCTGAACTCAAGTGATCTTCCTGCCTCAGCCTCCTGAGTAGCTGGGATTATAGGCCCCCAGCCCTGAGTGTCTTTTATCAATGGA
AGCAGGGCCTGGCCAGGCCCACCCCCTCTCCTCATGCCCTTGCAGAAGTGTCTGCATACACCCACCCCCAAGACACAGCTCATTCTTTCAACCT
GTCTGGGGAAGCCTGGCTGGGGTCCTGGCCTCTGCAAAGCCCACACCACCGCTCATTCCCATCCCTCTGATCCTAGCCTCCAGAGCTTTCTCCT
AAATTCCAGCCCTCCCCTACCACCCTTCTGCACAAGCAATGCCACTTCCCCAACTTGGCCTCTATTTCCCCATTTGGGGAAGAAGGAAGCTGTG
TACAAGGACCCAGGCAGCTCCAATGTCAGCAGCGGCAGACACCCTGCAGCCTTCCAAGCTGGAGGCAGGGGCATTTGGGGCACTGGGGCTGGCC
CTGGGGAGGCGGGCACTGGAAGTCACTGGTGTTGAGAAGATTCCAGAAGAGGCAGCTGTACCGCTGAGCCATGCAAGTGTCGGTGCTTGGGGTG
GAGGAAGGCAATGGCAAGTGGCACAGCAGAGGTGGGCGGTGTGTGGGGTGGAGCCACACTCTGGGGTCTGCACCCCAGGCTATCTGGGCAGCAG
AAGGAATTAGCTCTTGTCTGGTTTTACCTGCCTTCGTTCAGTTGTATGATTATTTTGAGATCCTTTGTGCTATTTTTCTTGTTATTCCTAAATT
AAATGCTGTTACAGACTTGCCACTGCTTTTTAACCATCCCTGTTAATGGACATTGGGGTACTTCCAGTTTGTGGCTTGTGTGAACATTTGCTTA
CAAGTCTTTGCACAGACACGGGTTTCCTCTGGGGCAAACCTAACGGCTGGTGGACGTTTACCCTTGAAGACACTGCCAAAGTGCCTCCCAAGGG
GACTGCACCTTTCCCCTCCATCCAGCTGTGAACAAGGGTCAGGGTCTGATTTCTCCACATCCTTGCCAACACTTGCTACTCTTTGACTCTTTGA
TTGCAGCCATCCCCGTAGGTGTGAGGCGGCCCCTTATTGGGTTTTTTGATTTGCACATCCCCAGGATGGGTGATGTCGACCATCTTTCCAGGTG
CATGCTGGCCTTCCTTGTGCCTTTTTTTTAAAGACAGGGTCTTGCTCTATCACCCATGCTGGAGTGTGACTATAGCTCACTGCAGCCTCAAACT
CCTGGGCTCTAGCAATCCTCCCACCTCCGCTTCCTGAATAGCTGGGACTACATGCACACACCACTGCACCTTGCTAATTTTAACATTTTTTTGT
AGAGATGGGGTCTTGCTACATTGCCAAGGCTGGTCTCAAACTCCTGGGCTCAAGCAATCCTCCCACCTCTGCCTCCCAAAGGGCTGGGATTACA
GATGTAAGCCACCTTGCCAGCTGTATGTTTTTTAAGACAGGGCTGGAGTGCAGTGGTGCTATCCTGGCTCACTGCAGCCTCCACTTCCTGGGCT
CAAGTGATCCTCCCGCCTCAGCCTCCCAAGTAGTTGGGGCCACATCCAGTTAATATTTTTAGTTTTTGTAGAGATGGGGTCTTGCTATGTTGAA
AAGGCTGTCCTGGTCTCAAGCAGTCCTCCTACTGTGCCTCTCAAAGTGCTGGGATTACAGGCATGAGCCACTGTACTTAGCCAAAGTTGCTTGT
TAAAAAGAGCCTGGTACCAACCTCCCCTCTCTCACTTCTGTCACCATGTAATTTCTTGCACACACTGGTTCCCCTTTACGCCCCCTTTCCCTTC
TGCCACAAGTGGAAACAGCCTGAGGCCCTTGCCAGAGGCAGTTGCTGGAGCCATGCTTCTGTGTAGCCTGTAGATCCGTGAACCAAGTAAGCCT
CTTTGCCTTATACACCACCCAACCTCACGAGTTCCTTTATAACAATGCAAATGGGCTAAGACCCCTGACCTGCCCAAGGCGGTCCTTTGAGCTG
AGCCCTGTTAACCCTGCATCTCTCCTCTCTCTACAGCTACCAGGGAGTCGGCCTTTGTCCACGCCATTGCCTCAGCCGGTGTGGCCTTTGCAGT
GACACGCTCATGTGCAGAAGGCACGGCCGCCATCTGTGGCTGCAGCAGCCGCCACCAGGGCTCACCAGGCAAGGGCTGGAAGTGGGGTGGCTGT
AGCGAGGACATCGAGTTTGGTGGGATGGTGTCTCGGGAGTTCGCCGACGCCCGGGAGAACCGGCCAGATGCCCGCTCAGCCATGAACCGCCACA
ACAACGAGGCTGGGCGCCAGGTAGGTTCGCCGCCCGCAAGGGTGCTTGGGAAAAAGGAGCCTCCTCAGCAGGGTGTGTGCCCTGGTTCCTTGGG
GCATATGGCCCGGTGAGGCAGGATGGTGGCCAGGCTGAGGGTCTTCTCGACCCCTGCCTGGGGTGTGCGAAGCTTAGCACCATCCAGCTACACC
ACAGCATCCAGTGCGCTCCTGGGGATGGCGAGCCCACCTGTGGACTGGGGTAGCCAAGAAGGAGTGATGAGGCAAAGGCCTGGTCAACAGGAGC
CCCGGCAGAGCCCAGGGAGGGAGACAGCCAGGCAGCCACAGAGACGGGGAGGAATTCTGCAATGAGGAAATGCAGGCACACAAGGTGACCCGAT
GTCAAGGACAGCTGGCAGTAATTTAGGGGGAGGGAGGCTGGGAGAGCCTCCCAGGCAGCCTGGAGGTGAAAGACAACAAGCGACTTGGTGGGCA
GAGCACAGAGTGTAATGGAACAAAGAGCATCTTGATCTGCAGAGGCCAACCCAAACCAAGCCGTGTAAAGCTTCGTGAGCCTGTGTCTTATTCT
GTCTGTGCTGCTACAACAAAATACCTTAGACTGGATAATTTATAAACAATGGGAGTTTAGTACTCACAGTTCTGGAGACTGGGAAGTCCAAGGT
CATGGTACTAGCAGATACCTTGTCCGGTGAGGGCCTGCTCCTCATGGATGATGCCTTCTGTGTCCTCACCCAGGGTGTGGATCGCTCTGGGGTT
ACTTTTATAAGGGCATTGTTTTAGGCCATTCTTGCATTGCTACCCGAGACTGGGTAATTTATGAAGAAAAGAGGTTTAATTGGCTCATGGATCT
GCAGGCTGCACAAGCTTGGTAACAGCATCTGCTCAGCTTCTTGGGAGGCCTCAGGGAGATTTTACACATGGCAGAAGTGGAGTGGTAGCAGGCA
CGTCACATGGGCCAGAGCAGGAGCAAGGAAGACAGAGTGGGGGAGAGGTCGCCACGCACAGATCTTGTGTGAACTCACTCATTATCATGAGGAC
AGCACAAGCCATTCATGAGGGATCTGCCCCCATAACCCAAACACCTCCTACCAGGTCCCACGCCCAACACTGGGGATTACACTTCAACATGAAA
TTTGGAGGGAACACAGATCCAAACCGTATCAGGCACGAATCCCATTCACGATGCTCCACCCTCATGACTTGACTTGATCACCTTCTAAAGGCCC
TACCTCTTAATGCTGTCACCTTGAGGGTAAAGATTTCTTTCTGGGGGGGTGGGGACACAGGGTCTTGCTGGATACAGTGGCACCATCTCAGCTC
ACTGCAGCCTCCACGTCCCGGGTTCAAGCGATTCTCCTGCCTCAGCCTTCTGAGTAGCTGGGACTATAGGCGCATGCCACCACACCTGGCTAAT
TTTTGTATTTTTAGTGGAGATAGGGTTTCACCATGTTGGCCAGGCTGGTCTTGAGCTTTTGGCCTCAAGTGATCCACCCACCTCGGCCTCCCAA
AGTGCTGTGATTACAGGTGTGAGCCACCATGCCCAGCCGAGGGTAAAGATTTTAACCCATGAATTTTTGAGGAACACAAACATTCAGACCACAG
CAGCTTGCTAGAAAGGATGTGGTCAGAAGATCAGTGGATGGCCAAGGGAGGAAAGATGGCTGATGGGCAGTGACGGGCGTAAGGACCAAGTGGC
GTCCACGGGATTTAACAACATGAAGATGGCAGCTGACCTTGGCTGTAGAATCAGGGGACCACAGAGGGGTGACGAGTGAACAGGGGATATTCAC
CTTTTAACAGTGAGGAACTAGATAACTCAGACTAATCTTCCTGCAGAAGACAACTTCAGATGCAGGATGAACTATAAAAAGCACCCTGTTAAAA
GTGTGGAGTCCCCATAATTACCCAATTCATGATAGATGAGGCAGTGCAGTGCCATGGGAGAAATGGGCTTTTCAATAAATGGTGCTCAGTCAAC
CGGGTGGTGCTATGGTCTGAATGTGTCTCCCAAAATTCGTATGTTGTAAGCTTGATCTCCAGTGCAACAGTCTGGAGAGGTGGGACCTAATAAG
AGGTGTTTAGGTGATGAGGGCCTTGCCCTCATAAAGGGACCAATGCTGCTGTAAAAGGGCTTGCAGAAGAGGGTCTCTCTCTCTCTCTCTTTCT
ATTACTTTCCTGCCATATGAGGACATGATGTTCCTCCCTTCTGGAGGACATGGCATTCATTCAGGGTGCCCTCTTGGAAGCAGATAAACCAAGC
CCTAACCTGTCAGCACCTTGATCTTAGATTTCCCAGCCTCCCAAACCATGAAAGAATACATTTATGAATTACTCAGTCTCAGGTGTTACGTTGT
AGCAATACAGCATTGACTAAGACAGATAGCAACATGGAAAAAAAATAACCCTGGCTTGCTACCTCACACCATCTGTAAAAATCAATTACAAATC
CCAGTGGGACAGGTAAAACAATAAAACTTTTACAAGAAAATATAGAATATCTTTGTGACCTTAGAGTAGGCAAAGGTGTCTTAAACAGTCCAAA
AAAGCACTCACATTGAAAGAGAAGTGGAATAACATAGACTATATTCAAATTAAGAACTTTCATCCATAAAAAGACACCACTATGGGACTGAAAA
GGCAACCCATAGAGGACACATTCACATACATACATCCACACTCACATATGCAGCAAATGTAGAGAAGCTACAGTAGGAAAAGAACTCTGCAGAC
TAAGAAAAAAAAGATAGACAAACCAATGGGAAAATTGGCCAAAAAATTAAAAGCCATGTTATGAAAGAAAATATAGAAATTCCACTACCTATAC
AAAAGGTGCTCAATGTTATGAGTCATCAAGAAAATGCTGTGAGAACCTATGTCCTGGAAAGAGAGAGGAGGATGGGGGTGTCTGAGACACTGGT
GTTCTGGGGCAGGTAATTTGTAAAATTTTGTATATTTTGCACTTATGACACATATATATTTTTGTATGTCTATTATACTTCCATAAAATGATTT
TTAAGTGTCAAATAACTGACCATCTAGTGAGGAATTAGCAGCCCCTGATCCAAGAGAAGATCCCAAGTCTGGTACCTGGGGTAGCTTTTGTCTA
GGGGCCATTTGCTGACCTGGAAGAGACTACAGAGGTTGCACTGCATTTCCAGCAGACTCACAGAGCTAAGGGGCCCACCAAGGGTGCCATCTCT
GGTAAATCCTTCCTCAGTGTAGGCTGTGACCCTGAAGAGCAAAGCCAAGCCAGAAGTAGTCTTGTCCTGTTCACATGGGCAGACTAGAAATCTC
TCAAAACCTGAACTTGGATTAAGGTAATCCCAAATTGCTGGTACTCCCATACACCTGGAAAAAGCAGACAAAATCCTCTTTGGAGAAGGATGAT
ATCCCTCCTCTAAGCCTATAAATGTTTCTATAATAATTTTTTAGAAAACACAATGTTGGCTGGGCGTGGTGGCTCACACCTATAATCCCAGCAC
TTTGGGAGGCCGAGGTGGGCGGATCGTGAGGTCAGGAGATTGAAACCATCCTGGCTAACATGGTGAAACCCGGTCTCTACTAAAAAATAACAAA
AAAAATTAGCCAGGCGTGGTGGCAGGTGCCTGTAGTCCCAGCTACTTGGGAGGCCGAGGCAGGAGAATGGCGTGAACCCAGGAGGCGGAGCTTG
CAGTGAGCCAAGATCGCACCACTGCACTCCAGCCTGGGCGACAGAGTGAGACTCTGTCTCAAAAAAAAAAAAAAAAAAAGAAAACACAATGTCC
AGCACACAATCAAAGATAATCTATCACATGATCTGATGACACAGTGAGTTAAACCAGAAAAAAAAAAAAAAACACAAGTACACAGGGAATTCAG
ACAATGGAATTATTAGACACAGACTTTGAGGGGAAAAATGCTTACTTAGGTTCACAAAGCTAAAACACAAGTGTGAACATTGCATCAGGAAACT
AGAAACTATAAAAATTATATAATAGGCTGGGCACGGTGGCTCGCACCTGTGATCCCAGCACTTTGGGAGGCCGAGGCGAGTTCGAGACTAGCCT
GACCAACATGGAGAATTCCCGTCTCTACTAAAAATACAAAATTAGCCGGGCGTGGTAGTGCACGCCTGTAATCCCAGCTACTTGGGAGGCTGAG
CCAGGAGAATCACTTGAATCCAGGAGGCGGAGGTTGTGGTGAGCTGAGAGTGCACCATTACACTCCAGCCTGGGCAGTTTTTTTTTTGGAAACT
GGGAGTTTTTTTTGGAAACTCCGTCCCAAAAAAAAAAAAAAAAAAAAAATATATATATATATATATATATATATATATATATATATATACACAC
ACACAATAGATTTGGAAAATAATTACACAGAAATTGCAGAACTGAAGAACAAATAAGGACTTCCAGTTAAAGATGAGAAGCTGAACACTTCCGT
CTGCACTCTCCTGGAACCCACTAAAATAACAATAAATTAATTTTAAAATAAATAGATATGTAGAGATAAATATAAATAAAATATAAATAGATAC
AGGTAGGGACAGGTAATAGTAGATATAGATGATAGATATAAATGGATATAAATAGTGCATAGAGATTATACATAGATATAGATGCTAATACAGA
TAGGATGGATGGATAAATAGATTAGATAGATGGATGAATAGATAGAGATGGATACTCATGAGAATACAGAAAATAAAGAGGAAACAATGGTAAC
AAAACTTTGAAAGCTAAAAGCAGATAGATGAATACTCAAGCAGACTCAAGAAAACTGAGTCTTAAGCCAGTAGAGAAGAAATTTGAGAACCAGT
CTAGGAATTCCTAGTGTCTGATATCTTGAGAAGTTGAGATAAATATGGGGCTGAAAACAGGAAGATTGGTTGAAAGATGACTTTCAGGGTACTT
TATAGCCCACTCACTCCACTACCCAACTCTTTGTAGCCAGGCAGCAGCCTCTCCTTTCCCTGTTGCTGAAGCCTGGAGGTTTGTTTTCTGCAAA
GCACAGGTCTCTGATCTGAGGGACGCCAAGCACATCTCAGAGTGAACCTTCCAAAGTGAAAATTTTCTCTCTACATATGGAAGTTCATGCCTTT
CTCTCTCCTCCTGATTTCCTAATGCTAGCTGTGAACCCTAGTTATCCTCCAGGCAAGAGGATGGAAGATTCTGCTCTGGGGACTCTGACCAACT
CAAAAGAGGAGACTTCAAGACAGTTACCTGTGGAAGGTCCCAATAAAGTGATGCAGCTAGATCACTCTATAGAGAGGACCAAAGTTGACAAGTC
TCTCCCATGTACAGGAACTTCCAATAAGATTTTTAGTGTCCCATTTTTAAATACGAGCAGACAGCCAAGGATCATCAGACCTCTGAGGAAGGAC
TCTAACAACAATAATGGCAAAGTCCAAAACAAACAAACTCAAACAAAATTTATGGGAAAGAGACATTGTAAAGAGAGATGAAACTTCGAAAGAT
GATCATTAATATCCCCAAAAGATAAAACACTATATCCATGAAATTGGAACAGGATGGTAATTTTAAACATTCAGGTTACAAAAGAGCTCTTAGA
AATTAAAAATATGATAACAGAAGCAAAATCCTCAAATTGAAAGATAGTTGAGATACCCTCCCACAAGACAGAAAGAAAAAAAAGACAAAGAGAT
TAAAAATAAGAGCAAGGAGAAGATTTGGAGGATCTGTCCAAGAAAGACAATGACAAACAAAAAGAGGAGGCAGTCAGCAAAGAAACAAGGCAGG
AAGCTTCCTCAGAACTGTGGAGCACAGGTTGCAGACCAAACAGGCCACTAAGTGACGAGAACAACAGAAGAAAAAGATCTACTTCAAGACACAC
TGCTGTGAAGCTCAGAGCCTACTGCACAGAACAGCCCTCCAAGCTGCCAAGGAGAATGTTTGGGCTTCAGGATCAGGAATCAAAATAGTACCAG
ACTTTTCGGGAGCAATAATGAAAGCTAGAAGAAAACAGTAATAATACTTTTTGAACTCCAAGATGTAAAAATGATTTTAAGCCAAGAATTCACT
TCCCAGCAAAACTATCAATTAAATGAGATAGTAGAAAAGATTGTTTTTAGACATGGACATTTCAAAAAATTTACAAGGCCTTTTCTGGGAAAAA
AAAATCTGCAAGAAAATGAAATTGATAGAATAGCTGATGTGTTTGGACATCTTGAAGAGAATATGTAGATAAGTAAGGGAGAATTTGAGGCTGA
ATCAGTTACAGGTACATTGATAACACCAACTACAAAAAGTCATTTAGGAAAAGAAAAGTCATCATAATATATGACACTTAAAGTAGTCCCAATA
CCACCAGCAGTGAATATTGATGTGAAATTAAGCTAGTTTTTAGGGCAGGAGGGGTTGGTGCTTGGGAAGTGTGCATCAGTGTGGTGGAGTAAGG
AGAGAAAACTAACTGCCACCCACCATGGCTAGAGGCCAATAAATAATGCCTAATGCTGAAAAATCAAGAGGTGTCAAGACAAGTGTGTTAACAG
AGTTGTAGACATTGCAAGGGGCTGCATGTAGGACCAGAATTCCTGTTTATCTAAACAGGTCTTGCAAACTGTTGGACTCTTCCAAGTAAGCACC
TATATAACTTTGATCCAATATAAAACTAAAACAAAAGAGGAAATAAAATGGATATTAGATAAACTAGACTGGGCAGGGCGAGGGATATGTTACT
CTACAGATGCCCATAGGAAACAGTCAATATCAGAGGGAGGAGTTCAAGAAAGAGCCAGAAGGGGACGAGGAGGAGGCAGGATTGGGACCCAGAG
CCCAGTTGAAGCCTTAGATTAGAACTGTCCAATAGAAATAGAATGGGAGCCACAAATGGCTCATAGATTATTATAATTTGTCCCACAAATGACA
CAAAGATCATTATAAATTTTCTAGTAGCCACATTTAAAGAGTAAACAGGTAAAACAAGTTTTTTTGGTTTTGATTTGGTTTGGTTTGGTTTGAG
ACAGACTCTCGCCCTGTAGCCCAGGCTGGATGGAGTGCAGTGGCGCAATCTCCGCTCACTGCTGCCTCCCCCTCCTGGCTCAAGCAATTCTGCC
TCTGCCTCCCAAGTAGCTGGGATCACAGGTTCCCGCCACCACGCCTAATTTTTGTATTTTTAATAGACACGTGGCTTTGCCTCAAGTGAGCCAC
CCGCCTTGGCCTCCCAAAGTGCTGGGATTACAGGCATGAGCCACCATGCCCGGCCAGAAACAAATTTTAATACCAAATTTGATTTAACTCAGGA
TAGCCAACATGCTATCATTTCAATAATGTAGTCAGTATTAAAATTATGAATGAGCTGTTTCTCATCCTTTTCTTACACTAAGTCTTGGGAATCC
AGCATGCAGTTGGCACGTATAGCATTTCTGAATTCACACTGCCATGCCTCATGGGCTCCTGTCTGTCCAGTGGCTGCCACAGTGCAGTGCCACC
TAGACTGTTCTGATGTTCACATGCCCCTTCACGCTCACCCCTACCCCTTCCCCAAAGAAAGAAGGGAAAAGAAGCTGAGTTCTACACTGCCCAT
AGGGGAAACCGTTCGCTCCTCAGCGAGGTCCTGCCCGGTCCCCCAACAGCCTTATGTCCCAACATTGCCTTCCTCTTCCTCTGCAGTCCCCATA
CAGTGACCAGCTTCCTGGGCCACTCTCTTTACTCATGCTCAGGACTCGTGCCTTTCCACCCAGCCGTATACACCTCTGGGCAGCGTGCCTGAGT
CCTCAGGGTCAGAAGGAACGCCTATCCCCCTGCATCCCTGCACTCTGACTGCTGTGGCTCTTCGTGTATGGCATCCCACGCTGCAGCTCTGTCC
TTCATTCGCTCCACCCTAGACAGTGTAGAAGTACTCAGCTAGGAGACGGGAAGAGGGGAGCCCCCCAAGCCCTGACCTGGGAAGGCTTGGGTCT
GGCAAGGGAGCAGGCACCAGTATCTGCACCATAGGAAGGGGGTGGCCCTGGGGCCCTGCCTGGGAGTCTGCCCCCTCTGCCCGGGGGCTTTATG
GAGCAGGTAGGCTGCAGGCGACATGTAATGCTGTTTCCTCGGGGAGACGCACCAGGGGGCCGCCCTGACGCTGGCTCCTGCGCGTGCCCCGCAG
GCCATCGCCAGCCACATGCACCTCAAGTGCAAGTGCCACGGGCTGTCGGGCAGCTGCGAGGTGAAGACATGCTGGTGGTCGCAACCCGACTTCC
GCGCCATCGGTGACTTCCTCAAGGACAAGTACGACAGCGCCTCGGAGATGGTGGTGGAGAAGCACCGGGAGTCCCGCGGCTGGGTGGAGACCCT
GCGGCCGCGCTACACCTACTTCAAGGTGCCCACGGAGCGCGACCTGGTCTACTACGAGGCCTCGCCCAACTTCTGCGAGCCCAACCCTGAGACG
GGCTCCTTCGGCACGCGCGACCGCACCTGCAACGTCAGCTCGCACGGCATCGACGGCTGCGACCTGCTGTGCTGCGGCCGCGGCCACAACGCGC
GAGCGGAGCGGCGCCGGGAGAAGTGCCGCTGCGTGTTCCACTGGTGCTGCTACGTCAGCTGCCAGGAGTGCACGCGCGTCTACGACGTGCACAC
CTGCAAGTAGGCACCGGCCGCGGCTCCCCCTGGACGGGGCGGGCCCTGCCTGAGGGTGGGCTTTTCCCTGGGTGGAGCAGGACTCCCACCTAAA
CGGGGCAGTACTCCTCCCTGGGGGCGGGACTCCTCCCTGGGGGTGGGGCTCCTACCTGGGGGCAGAACTCCTACCTGAAGGCAGGGCTCCTCCC
TGGAGCCAGTGTCTCCTCTCTGGTGGCTGGGCTGCTCCTGAATGAGGCGGAGCTCCAGGATGGGGAGGGGCTCTGCGTTGGCTTCTCCCTGGGG
ACGGGGCTCCCCTGGACAGAGGCGGGGCTACAGATTGGGCGGGGCTTCTCTTGGGTGGGACAGGGCTTCTCCTGCGGGGGCGAGGCCCCTCCCA
GTAAGGGCGTGGCTCTGGGTGGGCGGGGCACTAGGTAGGCTTCTACCTGCAGGCGGGGCTCCTCCTGAAGGAGGCGGGGCTCTAGGATGGGGCA
CGGCTCTGGGGTAGGCTGCTCCCTGAGGGCGGAGCGCCTCCTTAGGAGTGGGGTTTTATGGTGGATGAGGCTTCTTCCTGGATGGGGCAGAGCT
TCTCCTGACCAGGGCAAGGCCCCTTCCACGGGGGCTGTGGCTCTGGGTGGGCGTGGCCTGCATAGGCTCCTTCCTGTGGGTGGGGCTTCTCTGG
GACCAGGCTCCAATGGGGCGGGGCTTCTCTCCGCGGGTGGGACTCTTCCCTGGGAACCGCCCTCCTGATTAAGGCGTGGCTTCTGCAGGAATCC
CGGCTCCAGAGCAGGAAATTCAGCCCACCAGCCACCTCATCCCCAACCCCCTGTAAGGTTCCATCCACCCCTGCGTCGAGCTGGGAAGGTTCCA
TGAAGCGAGTCGGGTCCCCAACCCGTGCCCCTGGGATCCGAGGGCCCCTCTCCAAGCGCCTGGCTTTGGAATGCTCCAGGCGCGCCGACGCCTG
TGCCACCCCTTCCTCAGCCTGGGGTTTGACCACCCACCTGACCAGGGGCCCTACCTGGGGAAAGCCTGAAGGGCCTCCCAGCCCCCAACCCCAA
GACCAAGCTTAGTCCTGGGAGAGGACAGGGACTTCGCAGAGGCAAGCGACCGAGGCCCTCCCAAAGAGGCCCGCCCTGCCCGGGCTCCCACACC
GTCAGGTACTCCTGCCAGGGAACTGGCCTGCTGCGCCCCAGGCCCCGCCCGTCTCTGCTCTGCTCAGCTGCGCCCCCTTCTTTGCAGCTGCCCA
GCCCCTCCTCCCTGCCCTCGGGTCTCCCCACCTGCACTCCATCCAGCTACAGGAGAGATAGAAGCCTCTCGTCCCGTCCCTCCCTTTCCTCCGC
CTGTCCACAGCCCCTTAAGGGAAAGGTAGGAAGAGAGGTCCAGCCCCCCAGGCTGCCCAGAGCTGCTGGTCTCATTTGGGGGCGTTCGGGAGGT
TTGGGGGGCATCAACCCCCCGACTGTGCTGCTCGCGAAGGTCCCACAGCCCTGAGATGGGCCGGCCCCCTTCCTGGCCCCTCATGGCGGGACTG
GAGAAATGGTCCGCTTTCCTGGAGCCAATGGCCCGGCCCCTCCTGACTCATCCGCCTGGCCCGGGAATGAATGGGGAGGCCGCTGAACCCACCC
GGCCCATATCCCTGGTTGCCTCATGGCCAGCGCCCCTCAGCCTCTGCCACTGTGAACCGGCTCCCACCCTCAAGGTGCGGGGAGAAGAAGCGGC
CAGGCGGGGCGCCCCAAGAGCCCAAAAGAGGGCACACCGCCATCCTCTGCCTCAAATTCTGCGTTTTTGGTTTTAATGTTATATCTGATGCTGC
TATATCCACTGTCCAACGGAGTTAGACGAATAGATGTGTTGTGTTTTTTTTCCCTTTTCTTTCTATGAAAGAAATTGTTTTAGTCATAGCTTGT
GGGTTTCAAACAATGGGAAAAGTGAAAAAAGAGAAACAAGCCATCAAGGCTGTGGTCTGGGCTGGATGTTTGGTGGTGGGTCAGCACGGTGTTG
AAAGAGTAGAGAAGATGCCCACTCAGGGGCTCAGGGCCTGGGTCCGGGGGCAGGGGGATGCCCCAGGGCCGTGATGGGCAGGGGACCTCCAGAG
CTGGAATTCCATGGGGAAGGAATAAGGATAGCCAAGCTGAGGAAATGAGGGAACGGCCACTGGCACCAAGGCCTGGAGCAGAGTAGAGCCTCTG
ACAGGGAGAGAGCAGCAGGCACCTGGAGGCCCCAGGTAGGCAGCCCTAAGGTAAGGGGGTTGGCCGAGCCCCCAACAAGTCAGTAGAGCAGCAA
GACGCCCACCACCTCCCCTCCCGCAGAGCCTGTACTCACTCAGCTGATACCTTAGCACCAGCTATGGATGTGCCCCCTCTCTAGGTTTGGGGAC
AGTGGCCTGGTGGTATAGGATCAGCTGGATCCCAGCAGGACCTTGGGATGGGACTGGCCATCAGGAGTTCCTCTGGGATCCTCACCTCGTGGGG
CTGTGCACAGGGGTGCCCCCTGTGCGAGCCCCCATCTCCTTTCCCCAGCCAAGGAAGGCAGGGAGAGCCCCTGGAACAGGTGAGCCATCCTGCC
GACAGGAGCCCTCATGAGGCCTTACTAGGGGTGCTGGGTGTGCCTCTGGAAACAGGCAGCAGGAGCCCAAGGGCTGTCTCAAGGCCATGCCTTC
CCCGTCCCAGCCCCACCAAGGATCTCTGCTCCACTCTCTGCCCTCCCATGCCTGTCCAGTCCCCATGCCCATCTCTGGGCCCTGACTGGATGCT
CAGCCAGACCTTCAGGGCAGTGAATGCCAAGGGAAGAAGCTATGGAGATGGGGCAGCCAGGAGCAGCCAGTGTACCTGCCTGCAGGCCGGGCCC
AACACAGCCACCATACTCAAGCCATGTGCACGCCGGCGGGGAGCCAGGCCTTCATAGAACAGGAATGGCTGGCCATCCCCCAGTGTGCCCCATG
GTAGGAAGGGGGGTCAAGGCAGCCCCAGGAGTGGGGTGGGTGCACCAGCGTCCTAGGCATGGAGGTCATGGGAACACAATCCCCAGGCCTCAAG
AGCCAGAGAAGGGCCAGCCCCAGGCAGGAGCATAACCCCTCCAGGAACAGTGTCCCCTGACCCTGCCACGCACCTGCTGTCCTGGACCCTGAGG
GCACAGGCCCAGGGCATGCAGCTAAGGCTGGGCAGAGGGGCCTGAGGGACTGCTGACCTCCCTCCCCACACCCTCACCCCAAGGTGCCCTCACG
GCTGCCCAGCAGGACACCCTCTCTAGGCCACACTGGGCCTGGCCACCCTCTCTGGGCACCATTTTCAAGCTAGAGCCCTTGCTCCTGACCTTTG
TGACGGGTCCACTAGAAGCCTGGCCTTCCCAAACCCCCCACCTGCCCCTCACTATCCACTTCCTAGGTCCCCCTGGTCCTGTGCCTCATCCACC
TCCCTAAGGCTACAGATCCAGCCCGGCCCCCACCAGAGCCCTGCTCCTCCCCACCCCAGGTCAGGTCTTTCCACCCCACTCACTGCCCAGGGCC
TCCTCACAGTGCTGGCATAGTGAAGCCTCACAGCACCCACGGAGCAAGAGAGCATACCGCCCTCTACCATACCCTGCTGGCTGCACAGGGCTTC
TGCCCAGGTCTGGCTGCTGCCCAACCCCTGCCTGCAGCAGCCTTGCAGAACCTCATGAAGCAGAGGCTCCCACCCGAGGACAGTCAAGGAGGCC
AGGCCCAGGAGGGGATGAGGGCTGAAGTGTGGCTGCTGGGGTGGTTGTAGGGAGGGAGTGTTTCTCCAGAGCCACCAGCCATGGGAGCACAGAG
CTACCCAGGGCCTGTCAGGGCCAAGGGAGAAGGGACCCGGTCCAGGACTCTGGAGGTCAGGCACCCCTCTCCTTTGCCTCCCAAAACTGTGCCT
CAGCTTCCATGCACCCCCAAGGTCTGGTGGGCACCCCAATATCACTCCCAGCCATGGCCTCATTGGCTGGGGCCGTGACACCCATCCCAGGTGG
GTCACCCACTGCTCCAGGGCAGCCCTGACCCTCCATAGGCAGGGCCACTGGAATTCCCGGGGGGCACCAAGGGCAGCTGTGCTACCGTCTACAA
AGAGGGAGCCAGTGACTAAGGGCATAGGACCCCTGTGGTGCATCACCAGTGGGGGGACCCCAAGCCCGCTCAACCCGCCAGGCCTTCCTGAGCA
ATAGCCATAAAGGGTCAGTCCTTGGGGGAGGGCAGTGCCCCACAGGAGCCCCTGGTCAGCCCCAGCCCATGGGCACAGTGCCTCTTAGTCGGGA
CCCAGGTCACAGCCACCCTAATTAGCACCTACTGGGCCCCAGTGCCAGGGGCATTGCAGATGACAGACACTGACCCAGCCGGGAGGCGGCTGGG
CAGGCAGAGGGCACGAGATCATGGGCATGGCTGCCAGGTTGCAGGGACCTGAGCAGTCAGCATAGGCCGGGGCAAGAGCCCACACCCAGATCCA
GGCCAGGGCCAACTCGGGGCTCAAAGCTGAGGTAGGCCCCAGAGAGATCCCCAGCACCCAGGCTCTATCTCCGCTGCTGGGGCCCAGAGAGGAG
ATGGTGCTGGCCTGAGGACACACAGTCTGCTGGAGACCCAGGGGGCCATCTGGGTGGAGAGCCGAAGGTCCCCTTGCAGGCCAGGTGTGAAGGA
CCACTGTGAAGATGGTCTCAGCTGTCCCCATCCCCACTGTGGCCCTAGCACAGACAGAGGGTGACCCCACAAGAGGTTCTGGATGTGTTTCTGA
GGAATGGGGGTCGGCTGGTCTGCAGCCCCTGGGGCCATAGGACACACCCCAGTTTATAGGCAATGGGGGCTGGGAGGTGAAACGCTCAACTCAC
CCTGTGTAGGAGCAAGGCCCACTCCTGCCTGGGCCTCAGTTTCTTTTCTATGTAAAAGGCAGTGAGCCAAGGGAGGTCTTCCCTCCCACGGGCC
TGCTGGCTTGGGCTGCTGCACCTGGTGCATGGCCTCCCTCACCTGCCAAGGGCAGGCATCACCCCGGGAGCCCCAGCCCCCAACCCCCCCGGAG
CCTGCAGGCCTCCAAGTCAGTCTCAGAAGCCACCCCCCAGGGCGTTTATAGCCCATCCTGCCCACCCGCCCTGCAGAGGGAGGAAATGCGCCTG
GTGGGCCTGTTCGCAGGTAGTTGAGACAGGACAGGAATAACCATATAAAAGGCCCGGAGGTAGTGGGGCCCCACCCCACCAGGGCTCCCCTGCC
CAGCCCCCTGCACATCTCACCCTCCATGCTTTGAAGTCCTGGAGGGGTGTGGCCAGCCCCCAACTTGGGCACTCCTCCTCTCTCTGACACTCCC
CTCCTGACACCTACTTTCAGGATCCTGGGGCCTCAGTTTCCTCCCTTCAAGGAGGATCCTGGAGGCTGCCAGCCTGGCCTCCCCAGGACGCTGG
GACCGCAGCAGGCAGGTGCAAGGCCAGCTCCAGGTCCCCATGCCCGACATGGGGTGGGGGTGAGGCCCTACTTACTGACTGCCCAGTGCCCGTC
CCCAGTGCTGAGCCTGGGCTTGGTGTGCCCCGGGGCCCTGCCAGCCCTGGCCCTTGCCCCTGCTCCATCATGGGTCCTCTCTGGACCATGCTGG
GTCGGTGGGGGCCTGAGAGGCTCATCAGTGGAGACGGCCCCTCCCCTCTCAACCGGGTTCCACACCCAGCCTAACTCCCCTAGTGTGGGCCTGG
CGTCACCATCCTCCAGTGGGTGGGAGAGGAGAGGGCAGAGGGCAGGACCCCTGACCAAGCTCTGGGCCTGTCAGCTCCCAGGGGCACAGCTGCT
GCCCCAGGAGTTGACACAGCCCAAAAGATGACCTTGGCTCAGGGGGTCCTCGGCCTGTCCAAGGTTGGAATGTGGTCTGTCACGGCCTCCAGGG
AACTCCAGGACACAGGCTGAGCAGCCAGGAATGAATTCAGGGCTTCCCCGCAGTCCCTTGGGCTATCCTCAAGAAGGAACATTCTAGAAGCTTC
TTGTTGGGGGAAGGCGAGGCCTGGGGAAAGATGCCCCTGAAGAGGAAGAGAAGCTGGGACTTGGGGGTCCCACACCCCAGTCCTTGGAGGGGGT
CCTCCAAGGACCAGGGCATTGCAGAGATGATGTCAGGCCCAGCTTGCCGTCCAGGCCTCAATACGCCTGTTTGTGTATGGGGTGGAGTGTCTGA
GACGTCGTCAGACATCAGCCACTCCTAAGCCCTAGCAAGGCTGCCCTGGCCACAGGTCCCCCGATAAGGTGACCCTGTAACTTCATTCCCTGGA
GCCTTCCTCCAGCCTGGCCCTCCCCGATGTTTCAGCCGGATGGATACCTCCGCCCTTTCCCCTGCAGATAGCCTCTCACCAGGAATTACAATCA
GGGCCATGCACTCAGAGCCTGGCATCATCAGAGTGGGGGACCCAGGTCCCCGAGCCCAGGGCGGGGCTTGGTGGGTGGACTGTCCATCCTGCAC
TGAAGCCTGGAGTGACAAGCCATGAGAGAAAGTCCACGGGGAGAACCAGAGGGGGACTCCTGGGATGCGGAGCCAGCCTCTGCCCTCTGCCAGG
CTCTGTCCTGAGACCCTGCCCTTCCATCTGTAGGCCCTGCCATTCCTGGGCTACTGGCAGAGAGGGTAGAAGACGTTGGAGCACCCTGGGCTGC
GGGCCCAGGTAAAACAAGTACAGACCCTGAGCCTGGACTGAGCTGAGGATGTGCCCCATCCTGGAATATAAGAAGACACGGCCCTGCCCTGGGG
TCTGGGAGACACAGCCTCAACCTGGGTCCTGGGGGAAGCAGTCCCACCCTGGGGTCCAGCTGTCGACCTGGGATGAGCCCTAGTTCCCTCTGCC
CTACAGTGGCACCACCGCCCCCCACAGGCCCCACCCCCTCCCCTGGACAGCTCAGCCCTCTTTCCCAGTCTCCTCCCCTCATGGATCCCCCTTT
CTGCCTCTGAACCCCTAGGGATGGATGCCTCAGGACAGGGCACTCAGGTCCCTGTCGGAGTCTTGCTTGGATCTGGAGCCCTGGATCCCAGGGG
CATGACCTCCTGGAGCAACTTCCAAGGTCTGGGGGACTAGGTCAGTGTTCCTCCCTGCAGAACTGGACTGCTGTCAGTGGCTGCCTTCGGGATC
ATCTGGGAGGGCTTCCAGGAGGAGGTGAGCATGGGGACACTTCCTAGGGCTCCAACATCCTCCTGTAATTCTGAGATTGCATCCCTGCAGACGC
CAGGAAAAAAGTGGGTTCCCATGGCAGCCGGGGAGCCCTCAGAAGGGGCCCCACTGAAGCCTGGGAGCCACCACCCGCCTGCCTGGCCAGGATG
GGAGCTCAGCAGAAGGTGCTAATTGCTGTTTAGCTCCAAGCTGACTCCTTGCTCTCTTTTTGTCTCTCTCTCTCTCTCTCTTTTTTTTTTTTAC
TTGAGCCTCTAGAGCACACAGTAAACAGTCATTTGGCTCTTGGGGCCCTGCAAATTGGTGTTTATGGTGCGAAGATGAGGAGGATTTGTCCACC
CAAACACAGGCTGGCTGGGAGGGAGGAAGCAGGGGTGGGACATCCCATCCAGGCCCAGCTCGGGGAAGCCTCCTTCAGACTGAGGAGCTGTGGT
CTCACAAGCAGGGGACTGTGATTAACCCAGGAGAGCTTCTAGGAAGAGGTGAGGCATCCGCGGCAAAAAGACCTCCTCCAGGGGAAGCTGCAAA
GGCTCCACCATACCCTCAGGTGCCCATGGGGAGCCCTGCCCTCCAGACCCAGCTGGGATCAGAGCTTCTTGGCTGGGTCACAGCCTGGGTCTCC
TTCCAACAGCACACCTCACCTGCCCACCCAGCAGCCAGCTGCAGAATTACAAACTGGAATGCCAAGGCCCGGTCTCCTGGCAGGAGCAGGAAAC
ACAGAAGTCCAGGCGCAGGCATATGGGCTGGATGTGTGGCTGGGAAAAGTGGATTTCCCCAGGTATCTCCCCCTCGTTCACGAACCTGAGGTGG
GCAGACTATGCCCCAGAGCCAGTGCCCGGGCTCCTGTCTTCTTCTGCTGCCCAAACTCACCCCACCATGGCAGAAGGCCAAGAGGCCAGACTGG
CATTGCCCTCTCCCCACTCCATCCAAGGTCTGGCATGAGGCACCGGCCCTGCAGTGCCACTCCTCAATGGAGCAGGCAGCCTGCAGCACCAACA
CCAAAGGCTGGGGAGGACATGGGATGCTGGACATGGGAACCATCAGGAGTGTGCAGGAGGGGCAGTGTTGGCGGGTAGTGTAGACCAGAATGTA
CAGGGCATGTAGAAGAATGGATAGTGTAGACAGGGTAGTGCAGAGGAATAGCACAGGTGGGATAGTGTAGGGGGATAGTGTGGGGGGATAATGT
AGGGGGTATAGTGTAGAGGGATAGTGTAGGGGGATAGTGTGGGGGGATAATGTAGGGGGTATAGTGTAGAGGGATCGTGTAGGGGGAATAGCGT
AGGGGGGATAGTGTAGAGGGATAGTGTAGGGGATAGTGTATGGGGATAGTGTAAGGAGGATAGCATGGGGGATAGTGTANNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNTGCAGGGGTATAATGTAGGGGTATAGTGTAGGTGGATAATATT
GGGGGACAGTTTAGGGGGAGAGTGTAGGGTGACATTGCAGGGGTGATAGTGTAGAGGGATAGTGTAGAAGGATAGAGTACGGGGGATAGTGTAG
GCAGGAGAGTATAGGGGCATAGTGTAGGGGAGATAGTGTAGGGCAATAGCGTAGGGTGATATTGCCCAGGCTATAGTATAGGGGGTGCTGTAGA
GGGATAGTGTAGGGGAGGAAAGTGTAGGGGGGATGGTGTAGAGCGATAGTATAGGTGATAGTATAAAGGGATAGTGTAGTGGGGATAGTGTAGG
GGAACAGTGCAGGGGGGATACTGTACAGGGATAGTATGGGGGAGAGTGTAGGTGGATAGTGAAGGCGGATAGTGTGGGGCATAGTGCAGGTAGA
TAGTGTAGGACAATAGTGTAGGTGATAGTGTAGAGGGATAGTGTAGTGGGGATAGTGGAAGGGAATAGTGTAGGTGGATAGTGAAGGGGAATAG
TGTGGGGCATAGTGTAGAGGGATAGTGTAGGGGATAGTGCAGGGGTCACAGTGCTGTTGGGGTGCCAAGGCACCGTGCTCAGTGTCCATGATGG
TTTGTGTGCTTTGTGTCTGGAGCATCACGTGGGGACCCTGCAGACACAGAGTCCTGGTCACCCTCCCTGTTTCTGTGTCACTTGGCCTGCCTCA
GGTGGAGGGTCAGAGGTCAGTGTGTTCCCCGTTAGTGTGGGCACTTTCCTGGGCTGTGCCTAACCATAGGTGGCTCTGCTCAGGCCATCCTGGC
CCAGTGCTGCCTAAAGGTGTGCAGCTGGCCACCACCCACTCTGAAT
HUMAN SEQUENCE—mRNA
CGGCGATGGCCCCACTCGGATACTTCTTACTCCTCTGCAGCCTGAAGCAGGCTCTGGGCAGCTACCCGATCTGGTGGTCGCTGGCTGTTGGGCC
ACAGTATTCCTCCCTGGGCTCGCAGCCCATCCTGTGTGCCAGCATCCCGGGCCTGGTCCCCAAGCAGCTCCGCTTCTGCAGGAACTACGTGGAG
ATCATGCCCAGCGTGGCCGAGGGCATCAAGATTGGCATCCAGGAGTGCCAGCACCAGTTCCGCGGCCGCCGGTGGAACTGCACCACCGTCCACG
ACAGCCTGGCCATCTTCGGGCCCGTGCTGGACAAAGCTACCAGGGAGTCGGCCTTTGTCCACGCCATTGCCTCAGCCGGTGTGGCCTTTGCAGT
GACACGCTCATGTGCAGAAGGCACGGCCGCCATCTGTGGCTGCAGCAGCCGCCACCAGGGCTCACCAGGCAAGGGCTGGAAGTGGGGTGGCTGT
AGCGAGGACATCGAGTTTGGTGGGATGGTGTCTCGGGAGTTCGCCGACGCCCGGGAGAACCGGCCAGATGCCCGCTCAGCCATGAACCGCCACA
ACAACGAGGCTGGGCGCCAGGCCATCGCCAGCCACATGCACCTCAAGTGCAAGTGCCACGGGCTGTCGGGCAGCTGCGAGGTGAAGACATGCTG
GTGGTCGCAACCCGACTTCCGCGCCATCGGTGACTTCCTCAAGGACAAGTACGACAGCGCCTCGGAGATGGTGGTGGAGAAGCACCGGGAGTCC
CGCGGCTGGGTGGAGACCCTGCGGCCGCGCTACACCTACTTCAAGGTGCCCACGGAGCGCGACCTGGTCTACTACGAGGCCTCGCCCAACTTCT
GCGAGCCCAACCCTGAGACGGGCTCCTTCGGCACGCGCGACCGCACCTGCAACGTCAGCTCGCACGGCATCGACGGCTGCGACCTGCTGTGCTG
CGGCCGCGGCCACAACGCGCGAGCGGAGCGGCGCCGGGAGAAGTGCCGCTGCGTGTTCCACTGGTGCTGCTACGTCAGCTGCCAGGAGTGCACG
CGCGTCTACGACGTGCACACCTGCAAGTAGGCACCGGCCGCGGCTCCCCCTGGACGGGGCGGGCCCTGCCTGAGGGTGGGCTTTTCCCTGGGTG
GAGCAGGACTCCCACCTAAACGGGGCAGTACTCCTCCCTGGGGGCGGGACTCCTCCCTGGGGGTGGGGCTCCTACCTGGGGGCAGAACTCCTAC
CTGAAGGCAGGGCTCCTCCCTGGAGCTAGTGTCTCCTCTCTGGTGGCTGGGCTGCTCCTGAATGAGGCGGAGCTCCAGGATGGGGAGGGGCTCT
GCGTTGGCTTCTCCCTGGGGACGGGGCTCCCCTGGACAGAGGCGGGGCTACAGATTGGGCGGGGCTTCTCTTGGGTGGGACAGGGCTTCTCCTG
CGGGGGCGAGGCCCCTCCCAGTAAGGGCGTGGCTCTGGGTGGGCGGGGCACTAGGTAGGCTTCTACCTGCAGGCGGGGCTCCTCCTGAAGGAGG
CGGGGCTCTAGGATGGGGCACGGCTCTGGGGTAGGCTGCTCCCTGAGGGCG
HUMAN SEQUENCE—CODING
ATGGCCCCACTCGGATACTTCTTACTCCTCTGCAGCCTGAAGCAGGCTCTGGGCAGCTACCCGATCTGGTGGTCGCTGGCTGTTGGGCCACAGT
ATTCCTCCCTGGGCTCGCAGCCCATCCTGTGTGCCAGCATCCCGGGCCTGGTCCCCAAGCAGCTCCGCTTCTGCAGGAACTACGTGGAGATCAT
GCCCAGCGTGGCCGAGGGCATCAAGATTGGCATCCAGGAGTCCCAGCACCAGTTCCGCGGCCGCCGGTGGAACTGCACCACCGTCCACGACAGC
CTGGCCATCTTCGGGCCCGTGCTGGACAAAGCTACCAGGGAGTCGGCCTTTGTCCACGCCATTGCCTCAGCCGGTGTGGCCTTTGCAGTGACAC
GCTCATGTGCAGAAGGCACGGCCGCCATCTGTGGCTGCAGCAGCCGCCACCAGGGCTCACCAGGCAAGGGCTGGAAGTGGGGTGGCTGTAGCGA
GGACATCGAGTTTGGTGGGATGGTGTCTCGGGAGTTCGCCGACGCCCGGGAGAACCGGCCAGATGCCCGCTCAGCCATGAACCGCCACAACAAC
GAGGCTGGGCGCCAGGCCATCGCCAGCCACATGCACCTCAAGTGCAAGTGCCACGGGCTGTCGGGCAGCTGCGAGGTGAAGACATGCTGGTGGT
CGCAACCCGACTTCCGCGCCATCGGTGACTTCCTCAAGGACAAGTACGACAGCGCCTCGGAGATGGTGGTGGAGAAGCACCGGGAGTCCCGCGG
CTGGGTGGAGACCCTGCGGCCGCGCTACACCTACTTCAAGGTGCCCACGGAGCGCGACCTGGTCTACTACGAGGCCTCGCCCAACTTCTGCGAG
CCCAACCCTGAGACGGGCTCCTTCGGCACGCGCGACCGCACCTGCAACGTCAGCTCGCACGGCATCGACGGCTGCGACCTGCTGTGCTGCGGCC
GCGGCCACAACGCGCGAGCGGAGCGGCGCCGGGAGAAGTGCCGCTGCGTGTTCCACTGGTGCTGCTACGTCAGCTGCCAGGAGTGCACGCGCGT
CTACGACGTGCACACCTGCAAGTAG

TABLE 11

MOUSE NOMENCLATURE
ICSGNM Ly6e
Celera mCG2785
HUMAN NOMENCLATURE
HGNC LY6E
Celera hCG1765592
MOUSE SEQUENCE—GENOMIC
TCTCACTTCACCTGGGTCTTCCTCAGGACAGGGGCCTGCTTGGAAGAGCTTTGTGAGTGAAGTGGCTCATCCAGTGAGAAAGAAGAGACAGCCC
TGCCTGGGAACACCCTAGGAGCGAGCTTAGCAGACTGCAGATAAGGGTTCCTCCACCCTAGAGACACCCTCTGACTGTGGGAGGGACTTAAGGA
TTTTCTGACTGCACTAATCTTGTCCTAGTTTGTGAACTCGGCAAGGCACAGTGGACCAAACTGAAAAAGCAGGTACAGGAATGACTAAGGCTGA
GTTGAGAAACTCCTCACGGCTAGGACCAGGGCCTGCTAGGATCCACAGATGGATGGTTGGCCAACTTTCAGTCATCCTCATGAGATAACATTCT
GTCTGGGAAGGTCAAGGGGTTTGTGAGCAAGAAGTAGTACAGGTTGTATCCTGTCTCAGTCAATGTTCTATTGCTGTGAATAGACACCATGCCC
ACTCTTTACAAAAGAAAGCACTTAATTGGAGCTTGCTTACTGTGTCAGGGGTGTTGTCTATTATCACCATAGCGAGGAGCACGGCAACACAGGC
AAGCAGACAGGACGCTGGGGAAGTAGCGGAGAGTCCTGCATCTGGACCCACAGGCAAGCAGGAAGAGAGTGACACTGGGTCTCGCTTGGACCTT
TGATGCCTCAAAGCTCACCCCTCAGTGACACACTTCTTTGAACTGAAAGAACCTAGTGGGAAAACCCCCACTCAATTTCTGTTCAGTGCACACC
CAAGAATCACGAACAGACGACCATCTTGATGTAAAAGCATGAGGTAGTTTAATGACGGAGCTCCGGGCCGACACATATCTCCCACAGGAGACAG
AGGTGTGGACCACATGGTGGAGCCATAATGGCGGAGCTCCGGTTCAAAACGTATCTCACACAGGAGACAGTGGTTTCGACCATGAGGCTTGGAA
GCTAGGGGCTTTTATAGAAAAGGGGGTAGGGCTGGGGGAGGAATTAGCGAGGTTTTACATGATTGGTCTATTTAAATATCAACAGACTGTCAGA
AGGGCGGGAGATAGGGAGGCACTAGGCCAGTCAGGACATGTAAGGCCTGGGCATGTCCTGGCCTGTTCTGCTATGTTCTCAGCCCCAGGTTTTA
AAGCTCACAAACAACTCTTTGGGCTATTTTGACATAAATTACATGAATTCACATGTCTCAAGTTTTATTTCCTTTCAGAACAAGGCTANNNNNN
NNNNNNNNNNNNNNCCTGTCCTTACCCTGAAGACCATCCTGTCCCTGAAGACATACTGTCCTGGTCCCTGTAACTCCCTGGGGGGCAGCCGAAG
TAGGTGTTTCTATACATGAAGAGAGGACAGAAAGTTCTACATAAGACAACCTCAGCAAACTTCACCCTCACTGAACACATGTCTAGAATCCCAC
CAAGGTGGGACAGGCTCAGTAGTAACAGAACTCCAGCCCATAATAAAGCCATTGGCACAAGCCTAAGTCACACCATGGATGTCACTGTCAGCCC
ATGACAAGGGACCAGAAAGAGTAGCACAATTGAGTTGGAGATGTGGTTCTTAGCATGTATGAAGCCGTAGGTTTGATCCTTAGTACGCATAGCC
CTGGTATGGCGGTGCACAGCGCCAATCTGTACTCAGAAGCAGAGGCAGGAGGTAGGAGGATTAGATGTTCATCCTTAGCTACTCAGTAAGTTCT
AGACCACCCTGGGCTCCATCAGACCCTGGTTTGTTTGTTTGTTTGAATTTCTTGTTACCATTGCTCTGGAAGGGTAGATGTTTCAAACCCAAGA
AGACAGATGCCGGTGGATGGATTCTGAACTGTTTCCCAACAGCATCCGAAGGGTGGTTATAGAAACATATTCAGAAATAGAAAACAGTGGGGGT
GGGGGCTTCCGCAGGGGCCTGTGGTGTACCTGTTCAAGCGGCACTAGGATCTGAACATCTAGTCAGGTCACAAGGTCTGTGAGGTCTGGACTGA
GGCAGAGGTCACTGGCCACGCAAGTAAGATCTGAGTCCTTTGTTACAGAGGCTACAGAGAGACAATAGGAAGATTCCCACACATGACTTGGATG
GCCTAGGAATGAATGAGTGAACAGGAAGTGCTGAGCCACCAGTGGAGTGGGATCACAGATTTGTGAAGAGAGACAGCACCTGTCCCTCTCATGT
GGTGTCACAAGTGGACTGACTAAATTAAGGAATTGAAGGATAGACCAAGAGTTCAGCACATGAGATCAATCTGTGTAGAAACTCCTGGAATCAC
TGCCTGGGTCCTGAGATCACTGCCTTAAGGAGGGTTCTCCTTGGGGACGTGGATTCCAATGTCAGTCATAACTCCTAGAAGCTTATCACTCACT
CTCAGATTCCTCTTAGTTGCACAGCATTAAGGGAGCCAGCCAGACTCCTCTACACAATGCTTCAAAGGAGACTAGGCTTTGTGCCTGAGTAATG
ACAGAGACCTTAGAACTCTTGAGAGCTATATACATCCATAATCAATTAAGGTCTCTTCTGATGTAACTTGTCTTCCAGGTCCAAAGGAGGGGCC
TGTTCTCATGAATAATGGCTCCTCACTCACTGAGACCCTGCCATGAGCCACACGTGGTGCTAGACACAAGATGAAGTTCTCTGCCCTCAGGTCC
TTCATAGTGACAGCTTGACAGGAATTCAGATCATTGGAGCAGCCCAAGAGCAAGTATGAAAAAGATATTCTAACTACAAGGAAGACATCTCCAT
CCTTGGCTACTGGTCCTGCCCCCAGGTAAGACCAGGGAGGAAAGAGAAAGCTAAGGCCTCAGATACTGAGCAACAGCCTGGCCATTGCCTGCTG
GATGGTTTCTGTCTTGGGAACACCCCAAACTGTGGATTCCCAGACCACCAAATGTTACCATTGTTGTGTTGAGAGAGAGAGAGAGAGAGAGAGA
GAGAGAGAGAGAGAGAGAGAGAATATGATGTTTGGTGGAAGTGCAGTCAGGTGTGGGTGGAAGATGGTGCCTCTGAGGGCCCATGCTGAGCCAT
CCCTTCCCCATGAGGGTCCAGCCATAGATGGTATAGTATAGTACAGTATAGTATAATATAGTATAGAATAGAGTTTATTCAGGGCATGGGGAGG
GGGGGTTAAGAGGGTAGTNNNNNNNNNNNNNNNNNNNNCTTCCCTTTCCTTGCTGTGGCGTAAGCTAATTCCTGCATTACGGCTGGTGAGGCAT
CTACACATGCTTTAATATAATCTTTCAGGGGAACTTGTCTTATGGATTGGTCTGTTCAGGTCTTGGCACAAGGTATTCACGTATTCCCAAGCCA
ATTGTTTTATTAATATATCAGTCCCTTCCGAGGGGGGAAGCATCCTAGAAACTGCCTCTTCCAATCTAGCCACAAAACTCTCGTAAGATTCATC
ATGCTTTTGTCTAATTCCTGAAGGGACTGTGGATTTAGCCCCAGGTGGAGGTAAACTCTGCCAGCTAGATACAGCTATCTGACTAATTTTCTCC
AACACTGCTTTTGAAATTTTCAGTTGTGCTGCTCGTTTAGCATAGTCTTTTTGCCCAAGAAGCATAGGCACTGTGGGTTTAGTTCCATTTCTAT
TTTCCTCATGGAACTTCCTCATATCCTTACCTACTCGCTTCCAATATTCTAATGTCAAGCTTCCCTCCTCTAGAAACCAGGGACTGATTTTTTG
CACGAAATTATGAAATTTATCCGAAACCTTTAGGCTTCTTCCAGACAACATGTGCTTAAACACTGACAGAAACAATCTTTTCCCTTTTAACTTT
TACTGCCCCGTAATAAAAAATTACTCGTCTCTTAACGGATGTACAGATCCTCTCATCTGCAGCACGGTCACGACGAGATCCTATCTTTGCCTGA
GAAAATAAAGAATAGAAAGAAAAAAGAAAGAGAAAAAAGACTGATCTGCCCGGGTGTCCCTGTTTGAAGGCACCAACTGCTGCAGACCCTCTGG
GCTAATGTTTGTCTGCGTGAAAAGAGTCTCTCGAGCAGATGGGCAAAGAGTCACATGGAGTGACAAACAGATACACACACACACAAGAGAGGTG
TTGGATCTGAATGTAATGTTCCAGTCGAGCCTCAGACATTTTATAATACAGAGTAACAGAATAAGGCACGTTGAAGTTTTCCAGGTGCGAGAGG
AGTGACTTCAAAAGGAACCAGATAAATGTTTACTTTAGAGATTATCACCCTGCTTCAGATCAGCCCTGTGCCAGGCAAGAAATTTACACTTCAG
TGTTAATAGCACCAGGGGGTTGTTAACTCTTGACAGGCTTCAAGAATGATGTAGTGTCACTGACCCCGAAATTCTCTAGGCCTTGTTAAATTAT
ATCTAGTCTGGAGAATCCTCATTATCAGGATAGTATATTCACTTGCTCTTGGCATGGAATGTAGCTTGTGGTAAAATTTTTATCTCAGTGAGGA
TTTTTGTCTCTTCCTGCAGACAACTGAGTTAATGGCTGTAATTTATTCTTTTATTGTAGTGCTTAATTAGTTACTGAATAGGTTAAGCTCCTTG
CTGCTGTCTGGAATTTTGGAACACTGGGAAAGGCTTTAGCTATGTCAGAATACAATCTTAAAAGGCATTTACAATAAGGTAATACTAAATAGAG
AGCACGTGGATCCATACACTAGACTAACTCGGGAATGAAAAAATTAATTTTATGGTTTATATAGATTAGGGAGAGAGTGACAGACCCCAGGAAG
GGAGTTTCCTTGAAACTCTTTGCTTCATGAACCAGCTTCCCAGCTTTCGCTTTGTCAAGCTGACTCCACCAGAGTGCCATGGCAAACATGTGGT
CACTCAGATGCCTGTTGCTGCTGCTAAATACCTTCTTCACCATAAGGGACTCTTATCCCACCAGGAGCCCTGACCCTCAAATACACTCTTCCAT
AATGCTGTTTTCCATCCTGGTGTTTTATAACTGCAACAGAAAATTAATGGGATACATCCTGTCTTAAACAAAAGGAAAAAAAATAAAGCAAAAC
AATAACAAAAGTCATTCAAATAAAGCATTTTGGAAGCAAAACTAGGCAATAAAACTAAAACTGGAACAAAGACTTTTTTTTTTCAAGACAGGGT
TTCTCTGTGTAGCCCTGGATGTCCTGGAACTCACTCTGTAGACCAAGCTGGCCTCGAACTCAGAAATCCTCCTGCCTCTGCCTCCCAAGTGCTG
GGATTAAAGGCATGCACCACTACTGCCTGTCAGAACAAAGACTTCTAATTACTTCCTATTAAGAACAGACCAGCTGTTTCAAAACGGGGCATAG
GGGACAGTGTTAGATTTGGTCCTGTGTCTTCTCACAAGCTGCTTAGTTTCTTTACAGCCCTTCCCCCTTTTCATATACAAGGTCTCTTTTCATC
CTAGCTTGACCTGGACACTGAAGATGACCTTGAACTCCTGACCTCCTTGGCTCCACCTCCCAAATGCTTAGATGACAAGCACGCACTACCAGTC
CTGGTTTATATGGAGCTGGGGATGAACCCAGGGTGTCTTGCTTGCTAGGCAAGTACTCCAGCAAAGGAGCTGCATCTCTAGGCTCTCAATAGAC
TTCTTCAATCTTTCTACTTCATGATCTCTTTCACTCAAGGTGCAGTTCTTCTTTGCAGAGGAAGATAGTTTATTCTGGAACCATACAAAAATGG
CCCAGGATCACAGATTTAGGTTACCCCAAATTTCTGTGTTCCCAATGTGGCAACAGTTTGAGGAGNNNNNNNNNNNNNNNNNNNNGATAGGAGA
GGAGAGGAGAGGAGAGGAGAGGAGAGGAGAAATAGAGGCTGGCCATAAGCATGTGGAGAGGGGGAAGGGAATGGGAAGAGAAGGGGAATGAGTG
AGAGGACAGAGGGGTAGCAAGAGGGCTAGAGAGAGCAGAAGCGATGGCAAGAAGCCCCTTTTATAGTGAGTCGGGCATAACTGGCTGTTGCCAG
GTAACTGTGGGGTGGAGCCTAGACAAAACACTAACAGCCATGCTGTAGCCTGGACACCTGAGGACCCACACTGAGTCTTGCTGTCAGCTCTTGA
GGAATGAGCACCAAAAGCATGTGTGTCCCCAACCCTTATCTGTATGGGATGATCCTGGGCATGGCTGGAGCTGAGGAGAACTCTGAAAACACAC
AACAACAAGCTGTTTCTTCGCATCACTAGCAGCAAACACGGACTATGCTCCCAGTTCCCCTCCACCAACAGCTAGCTTCCAGCTACAGGGCAAC
TTGGCCTCGGCCCCACCCTGGCCAACCCACCCAAGCAGCTTCACAACTTTTTCTGTCTCCTCCCTACCCCCAATCCTAGGACCAGCCCCTGTCA
CCACCATCAACTCCTTAGACTGTGAGGAAGATCAGGTGCAAAGCCCCTATTTCCACATGTGAGACCAAGAATGGAAGTGTGACTCTCAGAAAAG
CAGGTGGAGGAAACTCAGCAGCCCACAATCCCCTGAGTAGGGCTGTGCTGGAGGATGATGGGGGTGAGGAGGGAAGGGAAGGAGAAGGAACAAA
CAGGACTGCCAGGCAAAGGGAGCCTGATGCCCTGATCTCCCCCAGAAGAATCACCCAGAAGCAGGACGTGGGTCCCAGGGTTCTGCTAGAGGGA
TAACACTCAGCAGGTGGTCGTTAATATTTGTTAGGTGTGGAAGCACTTCCTGAGCGGTAGGAATAGAATACTGGAGGACGCTTAGGTAAAAGCA
GAAGGGTTGGAGATTTCCTTGTAAAAACTGGGGCTTCAGCACTTAAGATGAGAACCAAAACCGGATGCCTGGGCTGGATCCTCTGTTCATACTC
AAGGAGCGGAGTGTAGGAGGCAGCCCGGCACCTAGAGGAAGTGGGAAGTGAGAAACAGATCTGTTCAAACCAGTGCTGCAATGACCTCCTTCCC
GAGGAATTTCTGTCCTGCCGCCTCATCCCCAAAAGAGGGATGGCCCAGACGGGCTCAGCCCAGAGGGACCTGACAACCTGAAGGGCGGGTGATG
AGGCATCCTTGGTCCAGGACTTCCTCAGCCTTACAGCAGCAGAGATTCCCATGAGCGTTTCAGGATGCAGTCTGTCCCCTCACTCCTTCCGGAA
GCAAGCTAAGCCTGCGCACAGGTCTTAGTATCGGTGATTGCCAGAAGCCCTGGCCTTTCCACCCTCCCTAGGTCTGTCCTGAAAGGTAAGGTAG
ACATGCTACACCAGAAGCCACGCCAGGGCTCTGATGCCACATTGTGTGCGGTGGGGCACTTAGGGCGGTCTCAGCCAGGCTTCTGGGTACCACC
GGGGACCGCCGCCGTGGGCGCATAGCTTGGGACCCCTGTCGTAGTCTCAGAGGAGCAGTTGTGCAGCCAAGCCAAGGGCACAGCCGGAGTTGCC
AGAGGCCAGCAGCCAGGGTCGTTCTCCCCACCAGCCAGAGACCCGCCCACAGCCCATGAAACGCCCCGCCTGCAGCCCGCCCCCGCCATCTGTG
CTCCGCTCCGCCTGCTTGGGTGCATTCAGCCTTTGGTGATCCTCCAGGACCTAAGGTTTCGGTATTATCTTCGGGGCCTCTTCACCCGGAGGTG
AGTCTGCTGAGCCCGACCTCAGGTGTTCACTCGTCACGCGAGGGCCTGGCCACGGCGGGGAAGCACTGGGAGAGAGCGGGAGGCCTTGACCGCA
GCGTGTAGGATCTGGGGTCCCGGCGCTCTGCTCCCGGGTGCAGAGTGGTTGGAGCATGCGAGGCCCTCTGCAGCGTGCTCTGTTCCTACCATCA
CCCCACCCCCACCCCAGTCCTTCCTGCACGTTTCTGGAGAGGCAAAGGCCCCTGCTGAGCCTTCACTGGGTACCCCTATCTCGACTCCACTTGT
CTGCTTGGGATCTCCAAAGGGGGATACACCCTCGTAAACTAGAGCAGGAGGCTGGGGGGTGGGGGTAGGGGTAGGGTCTGACCAGTTAGATTGA
GGCTGGACCCTCCCCAGAGTGAGGAGCGGGAAGCTCTTTGTCTTATCCAGGATTTTGCATATCAATGCTGAGGTTCTGGTGATTAAGTAGGGTC
CCTGGCTAGCGGGGGTAACCTGAACAACCTTCCTCCGCTGGCCGCTACCCAATAGACACCTGGCTTCCGCGGATGAGCCCTTTGAGTTTGTGGA
GGTGTTCCTCAGGTCCCCAGGGCCCTGCCCCGTTGCCTGCAGATGTGTGGACAGACAAAACATTGGACTCGTTTTCCAACCACTTCACCCCTTC
CTTCGCTAGTCCTCCGGTGGACGATGGGATGCCTCATTGACTTGTTTGTGGCTGTGCATGCCAGAGCTTCCTAGCAGACAGCTCAGGGCTCCAT
CGTTCCTAAATCCCACTGTGTAACGAAAAAAAAAAAAACAAAAACAACAACAACAACAACAACAAAAACAAAACCAGAAATGAGGACAGGCCCA
GCTGGGCTTGGCACTGAACTTGGCCACCTGGAGCTTTGGCTACCCACTACAAGATGTCAGCAAGTCTCAGGATAGAAGAGGCCTAAGGCCAGTC
CTGGAAGATACCAAAACTTCACCTCCTCTATACCCCGTTCCTCTAGGAGAACTGTAAGCCACCTTTCTCTCTGGGACGCCCCTCTCCCACTCAA
CAGATGGCACCAGTCATCTTCCTCCTCAAGAGGCCAGTGGTATTCAAAATAGGATATTGAAACCAAGCAAGCTGGTCCTCTCCTCTACCCTATG
CCAAAGACTTCTGCCCAAAGGGCCCAGAAAAGTCCCATAGCCATCTGGCTGGCTAGTGTGCTGACTGCTACAGGTGTCAGTGTCCCCAGCGAGC
AGGTAGTGGAGTGGAGGTGTCTTCTTGTGGAAATGGGTCCTGAGCCTCTACCTTGTACAGAAAGGAATTAGTGAAGCCCAGAGGCCTCCAGCCC
GTGTCTCTGCCACAAGAGAGAGGGGGTGGGGGTGGGGCTGTATTTGGTTCCCAGGGCTCAGGGAAGGTTTTCGGTTCATGCATGCTCATTTATC
TGACCACTGTCTTAACCCCAGGACAACCACTTAACTGTCCACTTCCATATCAGAGTTCTTGGTTCCCCTTTACTTCGTCTATGGAGCCCCTGAG
TTTGGGAAGGGGTATTAACTGAAAGGTACCATTAGATGAACTTGGAGAAAGATTTGTAGGTGCCACGGGAGATTTCAGGTAAAAGCTCTTTTAA
TAATTGGCTACAATAGCAGCAGGAGGAGGCCAGGAATTCTGAGGTAGAGATTTGATTAGCAGCACGTGGAGCAAAGGAGACTTCTGACTCCTAC
AGGTTTCAGAAAGTGGGGAGAGGCTCTCAGATGAATGGCTTGGACTGTGAGGTAAGTGTTAATGAAAGGAAATAAAACTGTAATTCATGTAATG
TATGTTAAATAGCCCCAAGAGTTGTTTCTGAGCTTTGAAACCTGGGGCTGAGAACATAGCAGAACAGACCAGGACATGCCCGGGCAAGCCCATC
GCCTCCCTAGCTCCCACCCCTCTGACCTAAGTTAAATGTTACAGGCTGCTGATGTTTAAATGGACCAATCATGTGAAACCGCGCCAATTCCTCC
CCCAGCCCCACTCCTTTTCTATAAAACCCCCTAGCTTCCAAGCCTCGTGGTCGAATCCACTGTCTCCTGTTGTGTGAGATACGTTTCGACCCGG
AGCTCCGCCATTAAAAAACCTCTTGTTGTTACATCAAGGTGTTGTGTTCTATTCGCGATTCTTGGGTGCACGCCGAATCGGGAGCTGAGTGGGG
GTTTCCCCACTGAGTTCTTTCATTAGTGTAGCACGAGACTAGCTTAAGGCTGTACAAATTGTCCGTCTGATTGGTGCATGGGGCCAGGTAGACA
CTAGATAAGCAATTGGGCCTTAACTCGAGTTGTCTTAATGGCTACCCCCAGGGCAGCAGGGAGTCAATACTGCTCTCCTGTCTGGGGCCTGAAT
GCTGAAACCATCTACAAAGGGGACATAAGGCATATTGGGGAGGTGTAGAGTGGGCCAGGCCCAGGGTCTGCTGGTATCTATGCCAGGATCCTGA
GTGGTGGTGGCGTAGCCTGTCTTTTAACCAACTGCCTCTCTCACAGTAGGCTTCTTTGGCTTGCGAACCTTCAGCAGATGTCTGCCACTTCCAA
CATGAGAGTCTTCCTGCCTGTGCTGTTGGCAGCCCTTCTGGGCATGGAGCAAGGTATGGAGCTCTGAGATAACCCTGCAGCCTGGTCCTCCTCC
TGATCTCTCATTCTTTCTCCTGAGTAGATGCCCAGGGCTCCTCCTGAGCCAGCCCTCCTAGGAACGTCTGGCCTTCCCACCTCCTACTCTCTAG
CCAACTGACCTAGTTCCCCTGATGCTGTGCTGGCCCAGCTACACCTTTGTCACCTCGTGTTGACTTTAGCCACTCCAGTACCAAAGTCTGAAGT
CAGTTGTCATTGGCTGGCTTTTTTCCCTGACCCAGGGCTGAGCATGCTGTTGCTTCTTGTCAGTGGGTTGGGGATCAAGGGCCCCACTGGAAAG
TCACCTTACCCTGGAAGGCTTCCCCAGGCAATAGGCAGGTGTCACCTACAGCATTGTCCCTTTTGCAGTTCATTCCCTGATGTGCTTCTCATGT
ACCGATCAGAAGAACAATATAAACTGCCTGTGGCCAGTTTCATGCCAGGAGAAAGACCATTACTGTATCACGTTATCTGCCGCTGCGGGCTTTG
GTGAGTAGCTGCCTGTTTCCCTAGCCAGGGCCAGGGACCTGCGGGGCTTTCCCCATTTCCTGCTACCCTGTCTGTCTGTCCCCTCATCCTCACT
TTCCATGCAGGGAATGTCAACCTTGGCTACACCCTGAACAAGGGCTGCTCCCCGATCTGCCCCAGTGAAAATGTCAATCTCAATCTCGGTGTGG
CGTCCGTGAACAGCTACTGCTGCCAAAGCTCCTTCTGCAACTTCAGCGCAGCTGGCCTCGGACTTCGTGCCAGTATCCCACTACTGGGCCTTGG
ACTCCTGCTTAGCTTGTTGGCTCTGCTGCAGCTGAGCCCCTGACCATCCCCCATGTGTTCTCCATATCCCCCCAGCTCAGGAAAAGCCCAGCTC
CTTTTAGGTCCCAGGGGACCCTAGGAACCTTCAGCTCCTCCTGGGTGTGTCTAGTTCCCCCTCCACACTCTCTCAACGTCAGGGCTAAGTACCA
AACTCACCCATACCTGCTCTGTTGAAGTGGTACTGGCTACCCTTGCTTCCAGAGCCAATCCTATAACCTCCCTTGGGGAACCAGCGAAGGGGTG
AAGATCTCCTTGGAGTCTCAAGAGTACCAGAGTCAGCGCCGAATCTTGTGGACACACTGACAAGGATGTCTAATCCAAATAGATGTATATCTGT
GTGCTCAGTGTATCCCTGTGTGAATGAAGCCACTTGGATTCTGGGGTGGGCAAAGAAGACCTGAAAAGATTCTACAGCAGAAGGCCTGTGTCGC
CACCAAAACCCCTCCCCCTGGTATCATTGTACCCACCTTGTACTCTGTTCAGGAGGCTGCCCATCGAGGACTGCCACCCCTCCAGATGAAGGCT
CCCACTACCCGATGCAGTTGAGTCCCATCCTGCCCTCTCTGCCCACACTGCCTTCCTGCTGCTATTCTAGTGCCTCAAATAAACCGTTCACACC
CTTTCCTGCTATGTCCTGGTTGTCCTTAACCCGTCTTCCCCCTCCGGTCCAAAGGGACCTGGGGATCCTGCAGGCCCACAGCCCTGCTTAAGCG
ATTCTGAAAGACCAACCCCGATACAGTGATACATTCCTATGATGCCATCATAGCAGAAGCAGAGGTCTCTGGGAGGCCAGCCTGGTGCATAGCA
AATCCCAGGAATCTAGGCCCATTTAGTGAGACAGGCCTGAGAGAAAAGAGAGAGAGCAAAGGCAGAGAGAACTTGAAAGTGTGCTGTCTCAGGC
TGAAAGCTGAGAAGTACAGCTACCCATAAAGGCAGGCCCATCAGGCTGGTCACACATTCCCAAGCTTTGTCTGCCCCAGAGACTTCAGAGGCCT
GCTGTTGCCACTTGTCCTTCATAAACTACCCTGTAGTCTAAGCCATGGGAGTCTGGCCCAGGCAAGTGATACTTGTCCACCCTATAACTGGCTT
TGCTTTGCTTTCTGGTATTTTAAGACGGTCTCGCTATGTTAAGACTAGCTGCCCCAAACCCAGAGCAATCCTCTTTGAGTGCTGGAATTAAAGG
CATGCACCACCACACCTGGCCCTTAGACTTTTTTGTTTCTGAGGCAGGATCTCACTTGAACGTTAGGTACTGGCATTACAGGCAGATGCTACTA
CACCTGGCTTCCTGGGGTTCAGTGCTGTGAGTATTATGGACACTGATGGAGAAGCTCCTGAGTCTGGGTGCCTAAGGAAGCTATTTCTCAGCTG
TTCCTAAGCTTCTGGGCTGTTGGGTGATACCATAATGGGATAAGATCCACACAGATCCTGGCTGTGGTGATATCCGTGTCTGAGGAATACTGCC
CAGTGGGGGTCAGACACAGGGACAACACAGGGGCTCAGGGTGAATTTCTAAAATCTATGTAGGCATTGGCTTTGTGCCATGATTCTCAAGACCA
GGGTTCCCATGACGTATAGCCTGAGGGTACAAGAAGGAATTAGCCTCATACCAGGTAGCTCAGCTACCTGAAAAGGACAAACTTTGGGGAAATG
TCACCGGGACAGCACCTGAGACTCAGAAAGTATGCTTTGTTCCTAGAATATTGAAAGGCCAAATGGAGAAAGGTGGGAGCCAAATCTTCTGCAA
GGAGCTTGGGCTGCCTCAGTGTAGACAGTATCCACTGTGGTGTTCAGGACAGATCTGAGTAAGGTTTCTAGAGACTGGCTTGTCTGTGGTCCCA
GCAGATCTAACCACCAAAAGCAGTATGAGGGAAGACACCTGGGTTAGACTCCCGGGACATGGGAACCAGAAGGGCACCAGGCAGTGACAGGGAC
ACGTAACAAGCTCTGGTTTTCTCCCTATCTTGATTTAGCTTCCATATTGTGATGGAGTGGTGTCACAAGGCTCAAGAGTTGAGGCCACACGTGA
TCTGGGATCCTCTGCTCTAGTCTGGAGGGCGTGATGGTTTCAAGGCTAAAAACTGGGACAGTGAGTGGTCTGCTCTGATTCCTCTCAGGAGAAG
AATGGGCAGAAGGGGCTTGAGTTCCAGGAATGACCTGAGGAGAAGGGAGGGTGAGGATTGGAAAGAGGAGGGAGAACTGGAAGCAGGGGAGGGA
GGGGCCAGGGGCCAAGTTTCACTTCCCAAAAGCAGAATCGAAAGTATCCCAGAGTTAAGTTTCAATTTCATGAAGCTTGGAGGCAGGAAGGGTG
TGGAAAGAGGGAGGAAGGGTGAATGGGGAGGGAAGGGTGCACACTGCACCTCTAAGAAGGCTGTCCAGGTTTGGAGCCACCAGCTTCACTCATA
CCAGATTCAAATGCTAGATCTGATCCCACATGATCCAATATGTCTCTGATTTGTTCTGGGCCCATTCTACCTCCAGACCCTGGCCAGCTTTCCT
ATTATTGCTGCTAGTTAGTGATCAGTGACCAGTGACCAGGGCTTCCCCAGTCTGATCCTGCTAGCCTGCCCCAACCAGCAAGTGCTTAGCTACC
TCCCTTCTGTCCCAAGGGCTCCAGTGACAGACTAGAGATTGAGGACTGCAACCTCAGGTGGACGCCTCTCCACCTGAGCATCATCCAACCAACT
GAGTATCCTCAGGCAAGATCAAAGCAAAAGATGAGAGAAATTTCCCTACTAGGGCTCCCTCTAATAAACAGAGCATATGCAGGAAAAGCCTACT
CTGTACCAAAGTATAAAAGGCAGGGGCGATGTAAAGGTCTCGTAAAGGGGTCTATACTTCATGGTGGATGCTTTGGGGGCTCTGTCCCCAGCTC
AGTAGCCCACTCTCATTCCTGGGAGTCTGTTGCTTGTCTAAACTCTATCTTTAATTCTAACCATCTGGTTCCAATCTTTTGTTCTGAGACATAG
GAGTCTAGAGTGCACTAATGCAAGAGGTGTTTTGTTTGTTTGTTTGTTTGTTTGTTTGTTTACTACTTTAAAAAATTGAATCTTATTTTTTTAT
TTTAATAAACTTTATTATTTAAAAAAGCGTGCAGATCAGGGGCTGGAAAGATGGCTCAGCAGTTAAGAGCACTGGCTGCTCTCCCAGAGGACCC
AGGTTCCATGTCCTGCACCCACATGGTGGCTCACAACTGTCTGTAATGCCAGTTTACAGGGATACCCATCCTCTTCTATCCTCTAGGGGCACTG
CATGCATGTGATACACAGACAGACAGACAGGTAGACAGACAGACAGACAGGCAAACCACTTAGACACATAAAATTAAAATCTTTTTTTTTAAAG
CTTTCAAACAAAACCACGATACATCAAACATTAACCAGACCCAAAGCCTAGACGACGTAATGAACTTACATGCAGTTAGAACTGGCATTTGAAA
TGGAAGCCGTGAAGCTCAGCATTAAATTTACACTTCCAGTACCAAACATTCTATGCATTGTTAGTTAGGGTTACCCTTGCTGTGATGAAACACC
ATCACCAAAAGCAATATGGGGAAGAGAGGGCTTATTTGACTTATACTTCCACATCATAGTGCATCACTGAAGGAAGTCAGGCGAGGCAGGAACC
TGGAGGCAGGAGCTGATGCAGAGGCCATGGAGGAGTGCTGCTTACTGGCTTGCCCCTCATGGCTTGTTCAGCCTGCTTGCTTTCTTCCTCCTCC
TCCTCCTCCTCCTCCTCCTCCTCCTCCTCCTCCTCGTCCTTTTTTTTTTGAGACAGGGTTTCTCTGTATAGCCCTGGCTGTCCTGGAACTCACT
CTGTAGACCAGGCTGGCCTCGAACTCAGAAATCTGCCTGCCTCTGCCTCCCAAGTGCTGGGATTAAAGGTGTGCACCATCACTGCCCGGCTCAA
TATGCTTTCTTAAAGGACCCACGACCGACTGCCTGCCCAGGGATGGCCCCACCTGCAATGGCCTGGGCCTTCCCCCGTCAATCAGCAAGAAAAT
GCTCTTCGGGGCTTGCCTACAGCGTGAAGAACGTAGGAAGGCATTTTCTTAATTGAGGTTTCCTCTTCATGGATGATTTAGCCTGTGTCACGTT
GACGTAAGACTATCCAGCCCACACCCTGACCCACTTTTCTTTAACTCCTTCCTCCCCTTGCCCATGATCTCTTATTTTGGAGACCTGCAGGTGC
TCAAGCTTGCTTTCCCTGACGCTGTGGCTGTTTCTCTGACCACTATGCCTAACTTTAAAGGTGTTAAAATTGCAGCGCTGGGTGTGGTGGTGCA
CGCCTTTAATCCCAGCACTTGGGAGGCAGAGGCAGGCAGATTTCTGAGTTCGAGGCCAGCCTGGCCTACAGAGTGAGTTCCAGGACAGCCAGGG
CTATACAGAGTAAACCCCTGTCTCAGAAAAAAAAAAAAAAAGATTGCAGCAAGTGCCTGGACAAGCAGAGTTTGGCCACCCAGCCGCAGAAAAG
AGCCAATGCAACCACATTGGCAAGAGAGACTAAGATCCAGTGGCCAGACCTCATTAAACTCATCCTTAGCGCCTTAACATGGGGGTAAGGTTTA
AACAGGAGACTGATAGCAAGGGTGAGGCAGTCCAGCCAAGGTGTGCCCCCCCCCCACCGCGGTCTCAGTTCTCAAGTCTACAGTGTCTCACAAG
TTATAAATCTTTCTCCAGGGGACATGTTCTTCCTCCTGTTTCCCCAGCCTTTGGCTCTTCCAGCAGGAAGTATCTGAGGAAATTCCAGTTTCTG
GGGATCCTTTATTTCAGTAATCTGCTAGCCCGAGAGAGGAACGCAGATAAATTTCTCCCAGGGAGGCAGGTTCCAAAGGACTGATTTGCCAGTC
CAGTCAAGTCTTCCCCTTGATCCTTAACTAAATTAAGTTGTCCTTGCGATCTTCTTGCTTTCCTTCTCAGTCTGGTCCACCCCCTATACACACC
TGCCCCAGTGATGAACTCTAAGGGTCCTTAGCCTGCTTCATCAGGCCTCATGGCCACAGCACCTCAGCTTCCAGAGGCCCTAGGTCCCTCTCCC
TCTCTTTCCATTCTGACAAGCCCTTTCCTGCAACACGTTTCATGTCAACTGGAGCACCCCAGGCAGCTTTGAGGCCTCAGGTGACTGGCTGACT
GGCACACAGTCCCATTAGGAAAGGCTGCCTGATTGCTGGAGGGCCTCTCGGCATACTCAGAACTCCGAGGACCACGGCTATAGCCCAGACCTCT
GGAGAAATTTAGAAGATGGAATGCTGGTCTGTGTACCGGGGCATAGCCAGTGGGGAATCCTTAGCCCAGCTGGCAGGAGTGATTGGTCTTACAG
GGCATGACCAGAATGACCAAAGTGCTATAGCTCTCCAAGGGGAAGCCAGAGTAAGACAGGCTGCCCCTGAGGTGGTGGCAGGTGGCAAGAAGCA
GAGGGCAGCTAGGAGCCCAAGTCGGGGCCACTGAGCAGGAAGTGGCTGTGGTCACGCGCATGCATACCGTACACCACTTGTTGGGGATCTGGGG
ATCTAGGTCCAGCAAAGAAAGCTTCAGGAGGAAAAACTCTTCCATGAAGTGTTACAGCTCAATAAAGCAGGCACCCTCAGTCAATCTTTGGTGA
AAATCACTCAGGCACTTTATTCCTTGTGGACTAGGACCTTATAAACATTTTGGGATGGGGTGAGTAAGTGCTTCCTATTGGCTTGGTTTGGGAT
TCTCTATCTGCATGGGGAAGGACTTCAGGTGTTGACTGTACATGCCTGATTTTCATGGTCCTCCTAGTGGGGTGTCTCAGTCACGTGCTCCAGG
ACAGGAATCTGGTTGGGAGCTAGTTTGAACTCCTGCCATTCTCAATGATGAATTTTATGGAAGTTCTGAACTTGCTACAACCTTACCAGCTCTT
CTATCTGGTGGCATGGCCCACTGCCCCCACAACTCCTCCTTTTCCTTTTTATAAAGGAGAGGTATCATTGGAGTGACTGTCTCCTGTGTTGGGG
ACAGTTTGGTATTGAACCAAAACCTGATTGGTAGTTATTTTTGCAATGCTACTAATCTGCTGCTTTAGAAATTGGATGAGACAGGGTGCCAAGA
GAAGCAAGGCTCCAAGCTCATACAGGGCCAAGGAGGAGCAGAAGCCAAGCTACCAAGGGGTTAGAGAGCCATGGGGCAGGGGTGTTGGGAGCAC
AGCATGCCCAGCGATCCCCCTGGAGCTCTTTGGGCATTTGCACATCTTGTTCTACCAGCCCAGATTCACTGACATAGAAGCAACATTCCTCTTC
TAGCAAAACACAAGTCCCTCCCTGTTCAGCAGGTAGTAAGCCCAAGGCCCTCCAGTTTTGGAGAACAACTCCTGCCAAGGAGGTTGTCTGGCAT
TGAAGGGACTCAAGCGTGTCTGCAGTGGAGGCCATTGCCTGGTCAAGTTTGGTCTGGAAGTCGATGGCCAGATGCTGTGTTTGGACCAGGGCTG
CTCTACCAGCTCCTGTCATGCCTAATGAGACAAGGAAACCCAGTGCAGCAAGCAGAGGGATGAAGATATCTCTCTCTCTCTCTCTCTCTCTCTC
TCTCTCTCTCTCTCTCTCTCTCTCTCTTCCTCCCATCTGCAAGGCAAGTTCTTCAGGTTCATAGAGATAAATCTGGGAAACAATTATTATAGGA
GTTGCAGGAGCCTTGAAAAGGGGTGGGGCGGGGGGCAGTGTTAATGCAGGAGCCTGTAGTGCTTTGCACCAGAGAAGTGCAGGGGGTTGATGTA
TGTTGATTTGGTGATGGTGTTGTAACAGGCACATATAAGATGGGGTTTTTGAGGAGGGTCCTACAAGGCAAAAGGGGGAAGAAATGCCCCACCT
TTAGGTTGAAGAGAGGAATATCCTGTGCTTTCCTTGTGGGTAAGGGTCTATGGGTTTTATGGGAACAGCTGCTAATAGCAGTCAGGAGAGAGAA
GCACAGAAAAAAGAGGAGTTTTTGGGAGGAGTTGGGGTGGGTCATTAAAGGAAGGAGGTCAGCTGTATATTGGATTTACTGTAGCCAGGAATGG
GTAGCTTGGAGTTCCTTCTGAGACTGATTTTCCCATTGAAGAATATTAACATGCATAAAGATTGATTTGGAGGTGGGAGGGGGGAGTTCACGAG
AGACATGTAAGTTGGCTATGGGGTATTTGGCATAGCCATTATGGTAGAGTTTACCCATGACTCCTCTTCGACAACATTCATCCCACAGATGGCT
GATGGAAAGCTGAAAATGAGTTTTAGGTTGGTGAGCATGGTGCCACTGTGGCTGGTTGGTTTCCACTCTGCAGCTCCAATACCTCCTGGAGACA
TTCACCTGTGTTTCAGTAAGGAAAGCAGAGGAAAGGGCTGTGTATAGTGGAGATGCCATCTGGGTGTAGGGAGATCTCAGTCCTGGTCCAACAT
GCTTCAAGTGGGCAATTCTGGGACCCTATGATTTTTGAGTGTGTATCTGTTGTAGGTCTCCCTAGAGAAGAATCTTTGATGGTAAGTGTTGGGA
AGGCTAGAGAGAGGCTGGACATTAGCGGAGTTAGTATGAAGAATGACAAAGGAGAGGTGTCCCCTTCCTCCAGGCTTGAGTAGGCTAAATAGAC
CTTAAGTGCTTGCCACAGTTGGACCTTAAAGACCCAGGTGGAATCTTCCATCTAGCCCATTTGCAATTTCTTACAGGAACAGACTGCCCGCTGA
GCTTACTTTGCAACTTAATCTAGCTGAAACAAAGGATGGGGTCTGTGGATTTTCCCTATATAAACACAGAGCTGAATATTAAATTTTGAGCCTT
GATCAGAAACTTTGTCTTGGCTTCATCCTTCTCTTGCACCCTATCCTTTATCATTTCTAGCCTCCCTTTCAAGTAGACCCAGTTCTCCATGGCT
GAAGGACAGCTACAGAGAAGGGAGGTTGTGTAAGGCACATGCCTGGGCAGCAGTCACCAGGTCATGAATGTCTTTCCAGGAGATCTTGTTCTTT
TGGGCATGGCATCTCCTCTGCCTCCTCCTCCACCTCCTCTGTCCTTGATCTGCCAGTAAGAGGCAGGCTATCTTCTTGGTTGGTGTTTGACATT
TCTGGCAGGAACCCAGATAGGAATAGGCTGATACTGTGGGAAAACACAAACGAAATCCTTCTCAGTTGTAAGGAGTCTGGTCCTTTCCAAATCC
CATCCAAAGGATCATGACATCGTACTAAGATGGGGGTGGGGGTGGGGGTAGGGGTGGTTAAATTTTGGTGTGTGCCAATGGAGGGAAATAGGAG
AATGCTTAGAGTTTTTGTAGATGTTAAATATGCTTGGTGTAGTCATAACTATTGTTAATTGTACATGGGGGTAGGTGGTTGGCTTTTGTTTTAG
AAGTTGAGCTTTTAGGATTTGATGTATTCTTTCTACTATCCCTTGGCCCTGAGGGTTGTGGGGAATCCCCGTATGGTGGGCAGTTTCCCATTGT
GTGCAGAAAGTTTTGAACTGAGAACCAGTGAAGACAGGGCCATTATTAGTTTCTTTTATCTGTTGAGGAATGCCCTCTGAAGCTAAGTTCACCA
AGAGCTGACAAAAAAAGGCTCTGTCAAAATCAAAGACACCCCAAATTTTCCTCTATCACCCCATCCTATCAGTTCTGATCAAGAAGTAGGGTCG
CCCTGGAGGAGAGCTCCAAGATGAAGTTTCTGACAGCTTTATTTTACCAAAGTCCAGTCTCCCAACCCAACCTCCATCCCAGAGAACAGGGTGT
TAAAGAAGGGACTTCCTTGACATTCCTTGGCACTTCTGAAGGACTGCCCCTCTTGTGAACCCTGTCCTCTAACCCCAAGTCTCTGAAGACTGTT
GATGAAAAGGGAACGCTGTTGTGCGGATAGATGTGGTGTAGGAAAACAGCTATGGAGACTCCAACTCCTGGGCAGGTTTGCCCGGATGTCTAAG
TAGGGAGGACAGAGGCTCCAGTGGGGGCCAGGAGTTCCAGCCAGAACTCAGCACCTGACTGTGAGGTGGGGCCTGACCCCTCTATGTGGACCAG
AAGTTCTCCCTGGGGTACCTTCCAACCTCCCAGCCTCTGGCCAGCTGTGCTATTTCCAGCCCCCAGCCTCCACCCTCAGAGGGGAGTGGTCTGG
GAATGTCACGGAGATGACATATACACTTCATCCCAGGCCTGGACAGAGGCCAGAGAGCCCTAAGAGGCTCTATTAGAGCCCCTGCTCCCTTCAC
TTCCGAAGGTCCCCATAGTCCTCTCCCTCCTCTAAACTTCAGGAAATCGAAACTCAAATTCTCCGCCTCTCCACCTCTTCCCAAAAGTCAGGAA
ACTGAAAGCTAGCTCCCGACCACCTCTGCCAATTTACTTTCTCTTCTCTCTCCCAGGTATCTTACAGCCTGTCTGAAGCCTGTGGTGCCCTGCC
CATGGTGAGTCCTAAGTAGGACCCAGAATCTAAGCAGGAAAGTGGAATGGGAGTTCTGCCCAGTGAAAGCTCCCTTTGAAGTAGCTCTGGCTCC
CCAAACACACAAGTCAGACTAAATCTTCCCTGGAGCCCTCAATGCCCGTATTACCCCCAAGCGCCCCTGGAAACTCCTATGGGTTGGACGGGAA
GGGTCGGGCATCCTCCTCTCGCCCCCAGGGAGGGACAGTGTTGTTAGTGAGTGTAATAGCTCTCAGCGTGTGCTTGCTATGGGGAGATTCCATT
GACTTGAAAGTTGAAATGCGCTCTTACTGCTTTGACAGCTTCTGCCACTAGGGGCGCGTGTGGCCAAGCCTGGAGCCCGGGTTTGAAAGCACCA
CCCTCCTGGGATAGGGTTAGTCAGCTTCTTCCGGCAAGCTGTGCCTGCCGGTGAATTAAAGGGGCAAGGGTGGCCACGGTGGCACATCCAGCTC
CCGTGCTAACCATCAGCTTAAAGTCATTAGATGAAAATGAGAACACAGCCACATGCCACAATTGAGTGTCCCCTGCCCTAGAGCAAACTCCCTT
CAGCCCTCACCCATAGGGCCTTTCCATCTGCTGGGCAGATGACTAAGGATCTTGGTCTAATAATTCCATCTTCCATTGATGAGTCTGCGGTTCC
ATCTTATGGCTTGAGTGTTGGAACATCTCTCGTGGAGGAGAAATGCACGGGCGCTAGGCTATGTTCCTTCCTCACAGCTGCCTTCCAGGGTGTC
GGGGTACCAGGCTCCTGGGACCCCAACACATCACTCAGGGGAGGTGAAATGCACTCTAAAATAGGGGTCCCAACCCATGTTTCTCCAGGTGAGA
AATGGCAGGGGCTAGGACAGTGGCTGTGGTTCTCAAACTCCTGGGCACCCAGATGCCGCCGAGAACGGCATTGAGAATGGGGTTCCCATACTTC
TCTGTATCCCAAGGCCGCTGAGGACTGCAAGCAGTTGGGAGAACAAGGGCCGAGGGCCTGTGATGAGGGCGTGTGGGGGCTCCAACTTAACTCA
GGTGAGTGCCAGGAGTGTGCAGGGGCCTTCTACCCGAGACTTAGGTGCAGCTCTGTATGATGAAGGCTTCCCCCCATAGTGGTCCTTGATGAAG
GAGACAGCCCTTGCTTGTCCAAACTGTTTATTCATGGCACATGAAGACTCATGTGATTTACTCAGGGTGAACCAGAGATTAAATACCTTTTGCA
GAAAGGGACGCTTAGGAAGAATGCTCATTGGCTAAACAC
MOUSE SEQUENCE—mRNA
CTTCACCCGGAGTAGGCTTCTTTGGCTTGCGAACCTTCAGCACATGTCTGCCACTTCCAACATGAGAGTCTTCCTGCCTGTGCTGTTGGCAGCC
CTTCTGGGCATGGAGCAAGTTCATTCCCTGATGTGCTTCTCATGTACCGATCAGAAGAACAATATAAACTGCCTGTGGCCAGTTTCATGCCAGG
AGAAAGACCATTACTGTATCACGTTATCTGCCGCTGCGGGCTTTGGGAATGTCAACCTTGGCTACACCCTGAACAAGGGCTGCTCCCCGATCTG
CCCCAGTGAAAATGTCAATCTCAATCTCGGTGTGGCGTCCGTGAACAGCTACTGCTGCCAAAGCTCCTTCTGCAACTTCAGCGCAGCTGGCCTC
GGACTTCGTGCCAGTATCCCACTACTGGGCCTTGGACTCCTGCTTAGCTTGTTGGCTCTGCTGCAGCTGAGCCCCTGACCATCCCCCATGTGTT
CTCCATATCCCCCCAGCTCAGGAAAAGCCCAGCTCCTTTTAGGTCCCAGGGGACCCTAGGAACCTTCAGCTCCTCCTGGGTGTGTCTAGTTCCC
CCTCCACACTCTCTCAACGTCAGGGCTAAGTACCAAACTCACCCATACCTGCTCTGTTGAAGTGGTACTGGCTACCCTTGCTTCCAGAGCCAAT
CCTATAACCTCCCTTGGGGAACCAGCGAAGGGGTGAAGATCTCCTTGGAGTCTCAAGAGTACCAGAGTCAGCGCCGAATCTTGTGGACACACTG
ACAAGGATGTCTAATCCAAATAGATGTATATCTGTGTGCTCAGTGTATCCCTGTGTGAATGAAGCCACTTGGATTCTGGGGTGGGCAAAGAAGA
CCTGAAAAGATTCTACAGCAGAAGGCCTGTGTCGCCACCAAAACCCCTCCCCCTGGTATCATTGTACCCACCTTGTACTCTGTTCAGGAGGCTG
CCCATGGAGGACTGCCACCCCTCCAGATGAAGGCTCCCACTACCCGATGCAGTTGAGTCCCATCCTGCCCTCTCTGCCCACACTGGCTTCCTGC
TGCTATTCTAGTGCCTCAAATAAACCGTTCACACCCTT
MOUSE SEQUENCE—CODING
ATGTCTGCCACTTCCAACATGAGAGTCTTCCTGCCTGTGCTGTTGGCAGCCCTTCTGGGCATGGAGCAAGTTCATTCCCTGATGTGCTTCTCAT
GTACCGATCAGAAGAACAATATAAACTGCCTGTGGCCAGTTTCATGCCAGGAGAAAGACCATTACTGTATCACGTTATCTGCCGCTGCGGGCTT
TGGGAATGTCAACCTTGGCTACACCCTGAACAAGGGCTGCTCCCCGATCTGCCCCAGTGAAAATGTCAATCTCAATCTCGGTGTGGCGTCCGTG
AACAGCTACTGCTGCCAAAGCTCCTTCTGCAACTTCAGCGCAGCTGGCCTCGGACTTCGTGCCAGTATCCCACTACTGGGCCTTGGACTCCTGC
TTAGCTTGTTGGCTCTGCTGCAGCTGAGCCCCTGA
HUMAN SEQUENCE—GENOMIC
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNTGGCAAAACCCCCATCTCTACAAAAATACAGAAATTAGCCAGGCGTGGTGGTAGTGCATGCCTGTAATCCCAGCT
ACTCAGGAGGCTGAGGCAGGAGAATCGCTTGAACCCGGGAGGCAGAGGTTGCGGTGAGTTGAGATTGCGCCACTGCACTCCAGCCTAGATAACA
GAGTGGGGCTCCATCTCAAAAAAAACACACACACACACACACAAATCAACTGTTCCTCTGGCCCCCACTTTCACCTGCAACCTGGAGAGCAGTG
GACACTGATCACAGCCTCACAAGAATATGACCCTCCCTTTTTTCCTTTCCTCCTTCCCCTGCGGCCACTTCTTTCTTTAACTATTGAAGCCCCA
AAACCCTCTTTGGAAAAGGAGTGGGCTGCACACCCTACTGTGTTTTCTGCCTCTTTTTCCCGGGCATGGCTTCAACCTTGGCAAAGTGAACCTC
TGAACTGGTTGAGACCTGTCTCAGTCACTCTGTGGGAGGGGCCTCCCCTGAGGTTGGGGAAGATGTCCCAAGAGGAGGGACAAGCAGCAACTCA
GGGAGGCCTGGAGCCAACTTCGGTTTCAGGGTTGAGCCAGGGTCAAGGTGGCAGTGGAAGTTACAGGAAGAGCCTAAGGCCCCAGTGGTGGGAG
CAGATGCTGAAATCCCCGGGAGCACTTTGGACTGTGCCCTGGGAGAGGCGGGAACCAAGCCTGAGAGCAGAGTACTGGCAGCTGCACGTTAGAA
TGTGAAGCCTGGACACAGGGAGGTCAGGCAGCGAGCTCACCTCACGGCAGCCCAGCCAGAGAGTCAGCCTGCTCTCCCCGCACTGAGCCAAGGC
TGGGAGACAGCCTACCTCCCACCAGAGCCAGCAGGCAGGTCCCAGCCCAGCCTGCCCCGTCCCGCCCCAGCCTGCCCTGTCCTGCCATCTGCAC
TCCCCGTCAGGCCTCCTGGCCAGCCCCACAGTCCCTGCCTTCCAAGAGACACAGGCAGCGGCAGCAGGGGCTGGATGGGGGAGGCCAAGAATCA
GAGGCGCTCCCCCACAGGACCCCTGAATCGTGGCCAGAGAAAGGTCAGGGCAGGATGAGGCCTGTGGCCACGGGCAGATCTCAGGTGGCTGACC
CGTCCTAGGAGCACACAGGGCAGCTGTGGCTCCTTCCCTCTTGAACCCAGAAAACAGGGCAGAGCTCATCGGTGGCCTCATCTGCCAGCCCCAG
CAGTAAGGCCACCGGCTCATTCTTCATGGGGCAGCAGCATCTGGCTTTCTGGCTCCAGACACCGCCTGCTGGGCCAGGCCTTCAACCAGGTCCC
TCCTGACAGCAGGCACGCCTGAGTCCAGACCCGAGACAGAGCCGGGAGGCAGCCTGCAGCAAACGTGCCCCAAACTCCACAGGCGCCTAACCCA
GTCCTGCCAAGGGAAAGTGCTCAAGGCTCTGCCCTCTCGCTGGAGGCCCACTGCTCACTCTCACCCGTGGGACTTCTCCCCACAGCCCCCAGCA
GCCTCCTCCCGTCATCTGAGACGCTACCAGGACAAAAGTCTTCCACAACTAGGGCCAAAGGCCTCTTAGTCATGGAAGTCTCCCTGAACCTATT
CTGGTTCGGAGGTTGCCTGGTTTAAAATTTTAAAAATTAAAAAAACAAAAGCCTCCGCATGACAGCAGGCCCGTCCCAGTCTTATGATCTCTCT
GCTCTGGTGTGGGAGGAGGAGGGAGTGCCGTGTGAGGAGGAGGGAGTGCCGTGTGAGGAGGAGGAGGGAGTCCTGTGTGAGGAGGAGGGAGTGC
CGTGTGAGGAGGAGGGAGTCCCGTGTGAGGAGGAGGGAGTCCCGTGTGAGGAGGAGGGAGTGCTGTGTGAGGAGGAGGAGGGAGTCCTGTGTGA
GGAGGAGGGAGTCCCATGTGAGGAGGAGGGAGTCCTGTGTGAGGAGGAAGGAGTCCCGTGTGAGGAGGAGGAGGGAGTCCTGTGTGAGGAGGAG
GGAGTGCTGTGTGAGGAGGAGGGAGTCCTGTGTGAGGAGGAAGGAGTCCCATGTGAGGAGGAGGAGGGAGTCCTGTGTGAGGAGGAGGGAGTGC
CGTGTGAGGAGGAGGAGGGAGTCCTGTGTGAGGGGGAGGGAGTCCTGTGTGAGGGGGGAGGGAGTCCTGTGTGAGGAGGAGGAGGGAGTGCTGT
GTGAGAAGGAGTGCTGCTACGTAAGAAGGAGGTAGATGTGTGGGGGGAGAGTTGGCATTCGGCCCGGTCAGGACTCCATGGCCCTTGTTACAGT
TGTGGGTAGAAATGGAATCTCTGTGACATCTCCTGCTCCCCTTTCGCCTGCCCCAAGGCAGGTCTCCAGTACTCCCTCTCCTTTCTGATCATGG
GCCCAAAGAGGCTCATTCAGAGAGGATCAGTCCCATGCCCTGGGGGCAGGAATGCTGAGCACATAGAGGTTTACCTGGAGGGGCCACGTGGGCG
GCATGGAAGCTCCCCACCCCTTCCGGGAGACGGCGCCCTGTGCCTCTCGCGTGTATCCATTTATAATTAACTGCAAATGTGTTTCCCTGAGTTC
CCTGAGCCACTCTTGCAAATCAATTGAACGCAAAACCGGGAGGGGCTGCAGTATTGCTGGGAAAGAGGGTCCTGATCCACACCCCAAGAGCAGG
TTCTTCGATGTTGTGCAGGAAGTAATTTGGGGTGAGCCACAGAGCACAGTGAGAGAAGCAGGTTTATTTGAAATGGCTCCGTTCCAGAGTGGGG
CGTGCTCAGAAAGCAGGAGGAGGAGGGCCTGTCCTCTGTTAGTGCCTCTGCTTACAAGAAACTGTAAGGAGCTACAATTACACCTGGAATGGGC
AGATGCACCCACTAAGGATAGGGGCTGTGGGTGCCATCAGTGCCCGTTCATCCTCCAACCTCAGGCTGCTCATTGAAATTATCTCTGAGTAAAG
TGGCCTGCACTCTTGGGTTATCTGGACAGTCTGCGGGCTTGCTGGGAGATGCCCCATATGGCCACAAATATTCTGTAATTATAATTGGTGTCCA
GCTTGAGACATGGCTATTTTTAGACCATAAGCATTAACCTTATAGGTGCCTTGTGAGTGCCTCCTACTCACTTCAAGATGGAGTCACTCTGGTC
ATGTTTCATTAAACCAGAGGCCTGGTGAGCAAGCGTTCCTCTAACAGTGGGAACCTCAACGTCGGGCCAGTCGGTAAGAAGTTCCAGAGGCTGG
CCCTCGCAGCTCATGTGTGTGCATATGTGTGTGTGTGTGTGTGTGTGTGTGTGTAGGTGTCTGGATCTGACGCTATCTGCAGGTACCGGAATGG
TGTTGGAGGACACTCGGCTGGTGTCCGCTGCAGAACTGATGGCTTGCTCGGTGGCGGGGAGANNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNGGGGAGGGGCAGGAGAGGGGCT
GGGGCTGCCGGTGGGTTGGTCCCCAGAGAGCTGAGCTTCTCCTCCCATCCCCCGCAGGCCTCCCCGAATGTTTCCAAAGATCTGGGGCGGGACC
GGGCGGAGACTGCCGCCAGGAGCCTCCCGGCCGCCCCAGGGCTGCGCAGCCACTGGAGCCCATCACCCAACTCCAGACGCTCCTGGCTCCTCTA
CGTGGGCCGGGAGGGACAGCCTTGAGGACTAGGGGAGGGGGCACGGGACCTTGCAGAGCTCCTGGCCGGAAAGGGAGGATTGACCGCCCCCGGC
ATATCACCCCGGAGCACTGGAACCCCCCCCCGCTTCTTGTTTTGGCACTGGTGGTGCTTGCGGTGAGGTCCAAGGAGCCCAGCCTCCCTGAGTG
GACCGCCGGGCCCCTCCCCGTTCCGGGACACAGGAGAGCTCCCGCCCCTTGCTGGCTGGGCAGCCCCTAGATACCTGGCTCCCAGGGGCCAGCT
TCCCTGAGCCTGGGGATGAGCCATGAGCCTGCAGCCTGGCCCCAGGGCGGCCCCCCACGGCCTGCCGGCTGGCTCCCTCCGGCTCCATGGCCCA
CCCGGCCTTCCTAATTGCCTTCGGCTCCCACCGGTGCCTTAGCTCAGCCTGGTGGCCCAGCGGGTTGGGTGCCACCCAGTGAGCAGGTGGCGGC
ACCAGCTGGACCTGTTTGTGGCCCTGTATGCTAGGACTTCCTCAGAGACAGCTCAGGGACCCCCCCCACCTACCAGAACCTCACTTAGGGGGTG
GGGGGAAAAGGAATTGGAGATCCTTCCTCCTCGGGCTACTCCCTGGAGGCGGGGATAGGCCCCAGCCGGACTTGGCACTGTTTTTGGCCACCTG
GGGCTCCCACTCCCAACCCCAGGATGTCAGCCCAGGTCTCACTGTCCTGGCCTGCTGCTCTCCCCTTAGGGCCTCTGCGGGCCCCTCTCCAGAT
CTGTTCTCTGAGGCATCCTCCTTACCCCCAGTGCCCAGCACTAGCTCCCCAGGCCCGGGATGTCCCTCCCACTCCTCTGCCCACGGCTGTCCCT
GACAGAAGGCAGCAGCCTCCCCCTCCAACACCATGCACTCACAAAACAGAGAATCACGACCCCAGCTGGGTGTGTTCCAGATTCTTTCTCCAGC
AGTGCAGAGGGTCCTGTGCAGAGGCCGAGGAGCAGTACAGCGACCCATCTGGCCCTTTCCTGCTGGTGGGACCAGTGGCACGCAGCCTTGTCTC
TCGGAGCCCATTTCCCAGCCACAGAATGGGGAGTAGCAGATACTGAGTGGGGTGCTTTCCTCGGAACTGAGTAACATCAGGAATGGGGAGTGCT
TTCCCCCTCAGCCATCCCCCACCTGGCCCCAGGTCTCCATGTGAGGGGAGCTGCCTGGGCTCAAAACACTGAGCCATCCCAAGGAAAATTCTAG
ATACAGATTAGCTAATATACACTGACAGATACACATATAGATGTGTATATAGATTTCTCTTTTTTTTGAGACAGGGTCTCACTCTGTTGCCCAG
GCTGCAGTGCAGTGGCATGATCATACTTCACTTCAGTCTTGAACTCCCTGGCTCAAGAGATCCTCCCACCTCAGCCTCCCAAGTAGCTGACCAC
AGGCACGTACCACGCCCGGCTAATTTTTGTATTTTTTGTAGAAACAGGAGGTCTCACTGTGTTGCCCAGGGTGGTCTCAAACCCCTGGCCTCAT
ATGATCCTCCTGCCTTGGCCTCACATAGCATGGAGATTACAGGCGTGAGCCCCTGTACCCAGCCCCAGACTAGCTAATATTATGACTGATCACC
ATTCCCCATTCCCCATTCCCCCACCCCAGGACCACTGGCAGAGGCCACTCACTCTGCCTTCTTTTCTGTGGTTCTGAGAAGGCTGCTGAGTTTC
CTCCTCTTGCCTGTGCAGCCCCCTCCTGCCAGGTTTCAGGAGGGAGATAGCCTAGAGCATGTCTGCACTGAGTGAGGAGTGGGTCAGGAGAGAA
GCAGGGAAGTCCTTGTGTGCTGGCAGGTCCCTTCCCCTCTGCTGTCTGTGTCCTCATCTGCAAAGTGGGGGTGCATGGTCTTGGGGAGGAGTGA
GGCACCACCCGGCCCCCTAACCAGTGTGTCTCTCCAGAGCAGGACAGGCTGCTTTGGTTTGTGACCTCCAGGCAGGACGGCCATCCTCTCCAGA
ATGAAGATCTTCTTGCCAGTGCTGCTGGCTGCCCTTCTGGGTGTGGAGCGAGGTGAGGTGCCCTTGGGGACCCCAGACCTTTGTCCAGCTGTGC
CCTGCTCCACTCCCTCTCCACCCCTCTCCCCTGAGCAGACGCTCCAGGGGTCCTTCCAGGCCGCTCCCAGCAGAGGGCTCACCCGGCCTGGCCA
CACTGTCTCACTGTGTGTTTGAGTGTCGCTTGACCTGCTCGACGGCCAGGGTGGGGTGTCACTGTCTTTGCTCTGCCTTCAGCCCAGGGCCTGG
TATACAGTAATTTCTCAGTAAATGTCCACTGGGGTCAGGCCTGGGAGGGACACTGGAGGCTTCCCTGAGACAGGTGTGTCCTCCTTCCGCAGCC
AGCTCGCTGATGTGCTTCTCCTGCTTGAACCAGAAGAGCAATCTGTACTGCCTGAAGCCGACCATCTGCTCCGACCAGGACAACTACTGCGTGA
CTGTGTCTGCTAGTGCCGGCATTGGTGAGTGCCAGGCCTCAGACCGTGCCTTCCTCCCCTGGCCATCTCCCTAGCCCGGGCCGGGGCTCAGCAG
AGGCCATTGCTGTCTGTCTGCAGCCGTCTGTCTCTCCCCTGACAGCCTCATTTCCCATGCAGGGAATCTCGTGACATTTGGCCACAGCCTGAGC
AAGACCTGTTCCCCGGCCTGCCCCATCCCAGAAGGCGTCAATGTTGGTGTGGCTTCCATGGGCATCAGCTGCTGCCAGAGCTTTCTGTGCAATT
TCAGTGCGGCCGATGGCGGGCTGCGGGCAAGCGTCACCCTGCTGGGTGCCGGGCTGCTGCTGAGCCTGCTGCCGGCCCTGCTGCGGTTTGGCCC
CTGACCGCCCAGACCCTGTCCCCCGATCCCCCAGCTCAGGAAGGAAAGCCCAGCCCTTTCTGGATCCCACAGTCTATGGGAGCCCCTGACTCCT
CACGTGCCTGATCTGTGCCCTTGGTCCCAGGTCAGGCCCACCCCCTGCACCTCCACCTGCCCCAGCCCCTGCCTCTCCCCCAAGTGGGGCCAGC
TGCCCTCACTTCTGGGGTGGATGATGTGACCTTCCTTGGGGGACTGCGGAAGGGACGAGGGTTCCCTGGAGTCTTACGGTCCAACATCAGGACC
AAGTCCCATGGACATGCTGACAGGGTCCCCAGGGAGACCGTGTCAGTAGGGATGTGTGCCTGGCTGTGTACGTGGGTGTGCAGTGCACGTGAGA
GCACGTGGCGGCTTCTGGGGGCCATGTTTGGGGAGGGAGGTGTGCCAGCAGCCTGGAGAGCCTCAGTCCCTGTAGCCCCCTGCCCTGGCACAGC
TGCATGCACTTCAAGGGCAGCCTTTGGGGGTTGGGGTTTCTGCCACTTCCGGGTCTAGGCCCTGCCCCAAATCCAGCCAGTCCTGCCCCAGCCC
ACCCCCACATTGGAGCCCTCCTGCTGCTTTGGTGCCTCAAATAAATACAGATGTCCCCCAGCTTCCTGCTCTGAGTGTGGCTGCCCCTTGCGGG
GAGAGGCAGAGCACCCCAGGTTTGGAGGGTCCTGGGGTCTTCTGTGGTATGGCCCAGGGGGGTGGTGGGGGAGGAGGAGTCATCCCCCTGAGCC
ACAGCCAGCTGCTTGCCTGACCTCAGAGGGAGCCCCTCCCCAGTGCTCTGCCCTTTCTTCTGCCCCAGGTTCAGCAGGTCAAGTGAGTTCCTCC
TCCACACAGCGGAAGGAGGATGTTGGGGGGCATGGAGGAAAGAAAGCGGGTGCAAGAAGGAGCACGCTCAGGCTGGGGCAGCACCCAGGGCCGT
AGGACTTGGGAATGGAGGGTGTCTGTCTGGACCCGCTGGTGCAGAGGGCACAAAAGCTGGGCTGGGGCAGGACAGACGGGCCGGAGTGTAGTGA
GTGGCCTGTGGGAGGGGCAGGGGGCTCCCACGCAGAAGCTCTGGGCAGCAGCCCTGACCCATCCGTGCCTGGACTGCAGCGCTGCCCGCTTCCC
CCTGGCAAACAGGTGGCAGCCTCCTGACAGAGGAAGCCGGCCCAGGCCGCCGTGTCTGCAGTTGGGTGCTGTGCGCTGCCCAGAGGGCTCTTTG
GGCTGGAGAGAGAAAACAAGAACAGACACCCAGTGGAGGTGTGTTTGTTCATTTCTCCGGGACAGTGGGAGGGAGCCGGCCAGGGTCTCAGCCA
CTTAGGGCCCGACCCTGAATTTTCCCGGCTGGCCAGAAACTGTGGTGTGTGTGTGTCAGGATCCCAGGGGCACCGTCCAGCAGGAGGGGCCGGT
GGTCAACGTGGAGGCCCGAGATTCCCACCCCTGGCAGCTGATGTGAATAGCCGGGTCCTCCCAGGTCCCTGAGGTCAAGGCAGGTCAAAGACCT
GTGTCCTCCTCCTGGGTCCCTGAGCTAGTGATTCCTGTCTGAAACACATGAATCATTACTGCCACCCATGAGGGGCCCATTGTAACCGAGCGAG
AGGGCCTGGAAGAGGTCAGCGGGAGGAGGGGCCTGATCGGTGGATCAGCTATCCCTGTGCAAGGACCCGGAGCCCATCAGAGCAGCCTGCAGAG
ACTGAGCTTGAGATTCCGTCGGGAGTTTGGATTCGCTGAGTGGGTCGCTCTAGCATTGAGAAATGCAAGCAGAGAACAGAGGGTGGAAACCAGA
TGGTCCAGGAGCCTGGGCACTGTAGCCAGGAGGACAGAGATCCCGGTGTGCAGTGCGTTCACTGCATTGTTCACTCGGAATCGCAGGTGGCTTT
GCTGGAGGGACAGACAGTTGATCATAGTGGAGCTGGTGAGAGGAAGCTGCACCCAAAAGCCATGGCCGGGAACCTGGATCTGACTGCAGGGAAA
CAGAAGCGTGGTGGGCGCTGGGCGATGATGGAATTTCTAACAAATGCTTTGTTTCTGCTAAAGCTAACTTGCGCTTCCATCTCTGGCATCTCCT
CCACTTCCCAGGTGAGCTGACTGGTTACTGAGCCTCACAACCACACTGAGACGGGGGCGCGGTCATCCCCTCGTTTCCCCAGGGGAGACAGGCT
CAGAGTGGAAGCCGCTTGTCCTCCTAGGATCTGGGACTCGGGGCCCAGCTCTCCCTCTGCTCTGGGTTTAGGGCTGAGGTCAGAGGCAGCCCTG
ACCTCCTGCTGGTGGTGGGTAGGGTAGTCCCGCCGGGTTCCTGCTGCCCCGCCCTCGGGAGGCTGGAGGCCCCCCAAGGACAGGTGGTTTCCAG
GAGCAGCGGGAGGGAGCCCCGGTTGTGTCCTGGGCCTGGAGCAGAGAGGGGAGGAGCCCAGCGCCTGAGTTTCAATTTCCTGAAGCCTGGGGGA
GGGAGAGGAAGAGAGCCTCGTTTCCTTTCGGGGCTCTGGACCTGAGCCTCCCGCCCAGGGCCCACCGCACCCCGCTAGGACCTGGGATTTCCCA
GTCTCCACACCGGCCCGGAACTGGGCCCCCTCCTCTCCGCAGCCCATCCAGCCCCACTTCCCCGCTTCCCCGCTTCCCAGCATCTGCCCGCGGA
TGAGCCTCTGGAGGATGGGCGGCCCGTCCCGGCGCTGCCCCTCACCCACCCCCCACCGCTCAACCCCTGTGGGGACTTCCTGCAGTGAGGCCCA
GGCAGGACGCTGCGCTGCCTCCCCCATCCACAGCCTCTGCCCCCTTGACACCCCCGCCCTCTGCCGTGCCTTGCAGGACTCCCCAGCCTGTGGG
CCCGGGACCATTTCACACGCTGTCCACAGTCGTGGAAACCCGGCAGCCACAGCAGGCCTGGGCCCACGGTGCCTCCACCCCTCCGTCAGCCCCC
AGCAAGTCTTCCTGGGGGTCCCCAGCCCTGAGTGCGAGACCCTCCAGGCTGCTTCTGGACCTCGGGGCTGGGGCTGAGCCCGATCCTGGCTGGA
AATGAGTGCAGGGCCGGGCTGCCTGGGGGAAGGCCCTCCAGTCTCTGAGCCCCACCCTGGGGAATTTGCTGGGACACCTTGAAGGCCGCAGGCC
AGACCCCAGGGGTTAATAAGGGAACAAAGTCCTTGGGCGAACACCTGCATTTACTATCTGAGCCAGCAGGCAGCTAGGATGCTGGGTGAGGGGG
TCTCTGGGCTGCTGGGCGGCCAGGCGGGGGCATCGGGGCAGGAGCTGAGGGCTGCACTGGGCAACCACCCTGAGCTCCTGTTTACCCACCAGGG
GCAGTGCCAGGGCGGGCTGGGCTCACAGCAGAGCCCAGAGGAAGGGGCTCAGGCTGAGCAGGAGCTCCCTGACAGCCTGGCACCAGAGGCCACG
CCGCACTGTGGCCGCCTTGTTACGGAGGCTATTTTCACAGCAGGTCATGTCCAGCTTCTCAATCTTCGAGTCAATCAGTCTATCCTCAATTTCT
GTGGGGTAAGACCCTCCAGGGACCACAGGATGGGTGAGGTTCATAATCTGGGCACAGAGTGGGGCCACAGCCCTTGCTAGTGTAGCTGAGCTTC
TGCCCTGTGGGCAAGAGAGGAGGGTGTCTGCCACAGGAGGCCCCACCCCACCCCCTCGGACCCGCCCCAGGACCGCCCCCTTCAGGGAACCTTC
AAGCCAGTGGGGACGCGGAGGCGAGCAGAAGAGAACGGGCGGCGGCCCCAGGCCCCACACCAAGTCTGCAGTCCATTAGATGGAGGTCCCTGAG
CTCTGTCCCCAGATCTAAGCTTCCACCAGGCCAGGGGGCTCAAGGCAGACCCCCTGGGATGCTGCCAGAGAGTGCCCTGCCCACCCCTCACCTG
CCTACAGACGCTGGGTCCCACCCAGGACTCACCATCGATAGTGCCAGTGACCACAGAGGAGGGGCAGCTTTGAGCCTGCATGGGATAGGAGACG
GGGTGACACAGGCTGGGGTCCAGGACACCGTAGCACTGGAAGCACTCAAGGCCCTGGCCTGGGATGGGGCGGGGAGGGTGCAGGTCAGGGTCGC
AGGCTGAAGGGTGGGTTCCAGTCCCTCTCACCAGCACCGGCAGCAGGCCTGGGCAGCACACAGGTAGGCTCAGGTGCCCTTGGCTACTCAGAAT
TGTGGGCAGGGTCCTGCTGGCATCTGGAGCCCCCAGCATCAAGAGGGTCTTACTCCCACCTTCAGGCCTCCTTCCCATCCCCCACCCTCAGCCA
GCAGCAGCATGGGGCTAGTGACCTTCATGGTCTTGGGTATGGGTGCATTGAGGGCACATGTGGCACAGAGGCTCTGAGCCTGGGAAGAGCTAGG
GGAGGCCTTGCACCCCCGAGCCTGGGAACCTCTGCCCCACTTCTGGTTTGTGATGAGGAACCCGGATTGTTTGTGTAAAGTGCACACAGCTGAT
GGCCTCATCCCATCATCTCACAGAATGGGAATTGAGGGGTAGAAACCCTTCCTTCCTGACCTGGCTGCTCTGGAAGGCAGACATGACCTTGAGC
AAGACCCCTCTCACCCCACCCCTTCTCTGCCTGGGGCCTCCCAGAGCTCAGGGTGTGAGCAGAACAGATCCGGAGGGAGGAGGGGCTGCGGTGA
GGAGAGGAGGGGCTGCGGTGAGGAGAGGAGGGAGAGGTTGTCCCCTGGGGTGGGAGGACGCCTTAGGAGAAGTTCTGACTCAATAGAGAGGAGG
GTCCTGGACTACGCCCTTGAGTTTCAGTTTCAGTTTCCTGGTGCATCACAGTGTTGGTGTCTGAACGAGGAGAGAAAAGCAGCCGTGAGACCTC
AGGTCACACCCAGGACCAGGCCCTTCCCGCTCAGGATGAGGGTCTCTGGCTGAGCCCACCACTGGCAGGCCCCCGTGCAGACAGAGCCTGGGAG
GGGCCAGCAGGGCAACAAGCTCTGACACTGAGACCCCAAGCCAGGCACCCCTTGTTTTCTGTGCAGGGAGCAGAGGCCCCTGTAACGGGGTGGG
ACGACATGGGGGCTCCCTTGGGGCAGGGGAGGAGGGNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNCCCACACCCGAA
TTTGTTCTTCACGGAGAATCAGAAGACACAGGAAATGGGCCAGGGGTGACAGACTGCAGACTCCACCCCCCCTCCCCAACCAGGTGCTGGGCAG
AAGGAAGACAGGCCTCCCTGGGCTGTGGGGACAGAGCCCTTGATGAAGACAGATAAAGGCAGCAGACCTGCATAAGGATGGGTCGCCTCTCCGG
TGGGTGGTCCCTTCCGAAAAGCAGAATCCACTCTGTGCCCTCAGGGGTCTCTGCTTGGAGACTCCATGGCATCTGAGAGGATGGGCCAGGTGAG
GTGGGGGTGGCTCCTGAGAGCAGGGAGGACAAGGGCGGAGCTGGAGGCGTCAGGCCCAGAAGACCCCCCCAGTGACTTCCGGCAGCTGTGGGCA
AGGCTTGGACCCAAGAGGCCTGACCACCCCGCCTGGCAGCACACCAGAGCACAGGCCAGACCTGAAGGGGCACGCCAGGGGGGGCCCATGGTCC
ACGGGTCACTCTGCTGGCCCTGCGGCCACACTGCTCAGGTCCCAGGGCCTGATGATGGCTGAGCCAGACATCATAACACCAAGCAATGGAGAAG
CAAGAATATTGCTAGGGATACTGACTTGTATTTTTCAATCTTAAAATATATGTTTTAATCTGACATCTATAAGCAGTGCATGTTACATGCCAGT
CAGAGCATGAGCATCCCCAAGAGGTGTGCACTCTGTGTATCTATGGATAATGAAAGCACGGCTTTCACACAGCAAGTTATCCTCACTTCCTGTG
ATCAACTCTGATATCTTCTACTTTATTTGAAAGAGTATTGATTGTGTCCCACTCGACTGATTTTACACCCTCTATGTACATGGTCCCCAGTTTA
TGGGCAGGGCGGTTATTTTGTGTTTTATTTTGTTTTTGAGACGGAGTCTTGCTCTGTCGCCCAGTCTGGAGTGCAGTGGCGTGATCTCGGCTCA
CTGCAACCTCCGCCTCCCCGGTTCCAGCGATTCTCCCGAGTAGCTGGGATTACAGGCGCCCGCCACCATGCCCGGCTAATTTTTTTATTTTTAG
TAGAGATGGGGTTTCACCATGTTAGGCTGGTCTCGAACTCCTGACTTCAAGTGATTTGCCTGCCTCAGCTTCCCAAAGTGCTGGGATTACAGGC
ATGAGCCACCACACCTGGCCTGTTATGTTTTTTGAGATAGGGTCTCCCTCTGTCACCCAGGCTAGAGTGCAGTGGTACAATCACAGCACATTGC
AGCCTCAACCTCCCTCCCAACCTCAACCTGCAGGGGAGGGGAGGGAGAGGAAGGGAGGCTCATCTCAGGGCTCTGAACCTGAGCCTCCCACCAG
GGATTTCCCAGTCTCCACACTGGCCTGGAACTGGGCGCCCTCCTCTCTGCAGCCCCATCCAGCCCCCACTTCCCAGCATCTGCCCACGGATGAG
CCTCCGGAGGATGGGCAGCAAGTCCTGGCACTGCCCCTCACCCGCCCCCCACCACTCAGCCCCTGTGGGGACCTCAGGAGGTAAGGCCCAGGCA
GGACCCTGTGCCACCTCCCCCATCCACAGCCTCTGCCCCCTTGACGCCCTTGTTACAGAGGCTCTTTTCACAGCAGGTCATGTCCAGCTTCTCA
GTCTTCAATCAATCAGTCCATTCTCAGTTTCTATGGGCTAAGACCCTCCAGGGACCACAGGATGGGTGACGTTCATAATCTGGACACACAGGGA
GGGCCACAGCCCTTGCTAGTGTAGCTGAACTTCTGCCCTGTGGGTGAAAGAGGAGGGTGTCTGGCACAGGAAGCCCCACCCGACCCCCTTGGTC
CCACCCCAGGACCGCCCCCTTCAGGGAACCTTGAAGCCAGTGGCGACACAGAGACAATCAGGAGACAAGCAGAAAAGAACAGGCCGCGCCGAGT
CTGCAGTCCTTCCAGCCTCAAGCGATCCTCCTGCCTCATCTTCCAAGTACCTCGGACCACAGGCACCTGCCACTACCCCCAGCTATTTAAAAAA
AAAAAATTTATAGAGATGGGATCTCACTATGTTGCCCAGGCTGATCTTGAACTCTTGGGCTCAAGTGATCCCCTTGCCTCAGCCTCCCAAAATG
CTGGAATTGTAAGCGTGGGCCTCTGCACCCGGCCTGGTCCGCAATTTAAAAACGCACACCCACCATTCCCTCTCCAGAAAGCACCCAGATGCCT
TTGGGAGAACCAGCCTCCTCCATGGAGGAAAGCTTGGCATCTGCCTTCCCACCTGGGGAGGAGAGGGATCTGTGGAAAATCCTTCTGACGGACT
TCCCCTCAGTGCCTGATCCATACTCAATAGTAGAAAAAGTAAGAAATATACAAAGATAGCAGATACACGGACACACTTCCCCAAATAGCTGAGC
GAATAGCGCAGAAGCAATATTGAAGACCTAATAGCTGAGACATTTCCAGAACTCATAAAGTGCATCCAGCCACAGATCAAGCAGCCCAGAAAAT
TCCAGGCAGCATCAACAAATAAATAGCCCCACATGCACCCCTGAAAATGCAGAAGACCAAACAAAAAAGTCCTGTCAACAGCCAGAGAAAAAGA
TCAGCCACCAGGGGTCGGTAGTGAGAAGGACAACTGTGGAAGCCAGGAGAGACGACAGCCCTGAGTTCCACAGGCAATGGAAGAAAGACTGTCA
GACAGAAAGGTTGTCCCCAATATACTTGCACTAAAAGAAAATCTTTTTTTAATTAATTAATTAATTTATTTATTTTAGACAGGATCTCACTTTG
GAGTGCAGTGGCGGGAACACAGCTTATTGTAGCCTTGACCCAGGCTCAAGCGATCCTCCCACCTCTGTCCCCGAAGTAGCTAGGATGACAGGTG
TGCACCACCATCCCCAGCTAATTTTTGTATTTTCATACAGGCGGGGTTTCTCCCTGTTGCCCCACCTGCTCTTGAACTCCCTTGATCAAGGAAT
GCTGGGACTACAGGTGTGAGCCACAGCACCGCCCAAGAAGGTCTTTTAAAAAGTCTTTTCGGGCACGGTGGCTCATGCCTGTAATTCCAGCACT
TTGGGAGGCCAAGGCGGGTGGATCACAAGGTCAGCAGTTCAAGACCAGCCTTCCCAAGACGGTGAAACCCCGTCTCTACTAAAATTAGCTGAGT
GTGGTGGTGGGCACCTGTAATCCCGGCTACTCGGGAGGCTGAGGTAGCGAATTGCTTGAACCCGGAGGCAGAAGTTGCAGTGAGCCAAGATCAC
GCCACTGCACTCCAGCCTGGCGACAGAGACAGACTCTGTCTCAAACAAAAAAAAGAAAAAGAAAAGAAAAGTGATCTTTAGGCTAGACATGGTG
CATGGTGGCTCTCACCTGTAATCCCAGCACTTTGGGAGGCCTAGGCAGAAGGATCGCTTGAGTCCAGGAGTTCCAGACCAGCCTGACCAACATG
GAGAAACCTGTCTCTACTAAAAATACAAAAAAATTAGCCGGGCGTGGTGGCGCATGCGGGTAGCCCCAGCTACTCAGGACGCTGAGGCAGGAGA
ATCGCTTGAACCCAGGAGGCAGAGGTTGTGGTGAGCTGAGATTGTGCCATTGCGCTCCAGCCTGGGCAACAAGAGCAAAACTCCATCTCAAAAA
AAAAAAAAAAAGTGATCTTTAGGCCGGGCACAGTGGCTTGTGCCTGTAATCCCAGCACTTTGGGAGGCTCAGGTGGGTGAATCACTTAAGGTCA
GGAGTTGAAGACCAGCCTGGCCAACATGGTGAAACCCCATCTTTACCAAAAATACAAAAATTAGCCGGGTGTGGTGGCACGCACCTGTGATCCC
AGCTACTTGGGAGGCTGAAGCAGGAGAACCACTTGAACCCAGGAGGCGGAGGTTGCAGCGAGCCAAGATCTCGCCACTGCACGCCAGCCTGGGC
AACAGAGCGAGACTACATCTCAGAAAAAAAAACAGTGATCTTTAGCCAGAAGGAAAATGGTCCCATTCAGAATCAGAGATACCTCAAGTGATGA
AGCACAGATTCGTAGAAATGTGGTGAGTCTAAATGAACATCAATTCTATTAAAAAAGATAAAATAGTGGCCGGCGCGGTGGCTTACGCCTGTAA
TCCCAGCACTTTGGGAGGCCGAGCGCGGCGGATCACCTGAGGTCAGGAGTTCAAGACCAGCCTGACCAACATGGTGAAACTCCATCTCTACTAA
AAATACAAAAAAAATTAGCAAGGTGTGGTCGCACATGCCTGTAATCCCAGCTACTACGGAGGCTGAGATGGGAGAATCACTTGAACCCAGAAGG
CCTCTCACGCCATTGCATTCCAGCCTGGGCGACAGAGCAAGACTCCGTCTCAAAAAAAAAAAAAAAGTTCTGTCTTGTGGGAGATTTAAGTTTT
TGTGGTGAAATTAAAATTCATACCATAAATACAAACCTGGAAAGGAGTGAATACAATGACGCCAACCCACTGGGCGGTTACACAGTGGCCCACA
GCTGTAGTCCCCACTACTCAGAAGGCTGAGGCAGGAGGACGGCTGGAGCCCAGGAGTTCAAGACCCACATGGGCAACATAGTGAGATGGAGCAG
GGACCCCTCCTACGTGCCTGCCAGGCTCTCCCAAGCTTGGAAATCAAGGAACACTCTTGAATCCCTTCAAGGGAAATTCCAGTCACCTTGCCAG
CCTTGAAAGGTAAGTGAGCAGCCTGCCAGGAAGGCGACGGGAGCACGCGTCTCCCAAGCAGGCCACAGCCACAGGTGGTTTGCTTCCCTATAGA
AACTAAAGCATAACATACATTCTCAAGTCTTTCAGAAACCCCCACCCGGTGGAAAATGCCGACTGCTATCACAGAGACCCAGATAAGGGGGAAC
TGAGGACTGAACTCTGGCCGTGTTCTCTGTCATGAATTTCTTCTTGAGGGGCCTGGAGGGAGTCACAGCTGTGAGCCACTAATGTGTGCCCCCA
TGCACACAGCACCCTTGGCATAACTAACTCCATCTTAGAGACTCCATTGTATATCTTACAGGGCACTTGGCCAACAAAGGTAAGATGTTTTGCT
TAATAAACAAGTTAAAAATAAAGCCTGCATCCAAACAGATGAGGACGCAAGCAAGCGCACTCTTCAGATGTCACTTCTCACGGGAGGCCTCTGT
GATTGCAGAAGGAAAGGCCCCAGCAGAACTCACCATCTGCCGCCTGAGGCTCCACCACCTCGAAGACATCCTTGCAAGATCCACAGACTGGCCC
AGACCAGGAGGTTTCTGTCTTCTTCTTGTGTCTTCTTCACTCTCCTGGACTGGTTCGTGAACCCTTTCTCCTATCTCTGTTTCCTCTGGATGTT
HUMAN SEQUENCE—mRNA
GCTCCGGCCAGCCGCGGTCCAGAGCGCGCGAGGTTCGGGGAGCTCCGCCAGGCTGCTGGTACCTGCGTCCGCCCGGCGAGCAGGACAGGCTGCT
TTGGTTTGTGACCTCCAGGCAGGACGGCCATCCTCTCCAGAATGAAGATCTTCTTGCCAGTGCTGCTGGCTGCCCTTCTGGGTGTGGAGCGAGC
CAGCTCGCTGATGTGCTTCTCCTCCTTGAACCAGAAGAGCAATCTGTACTGCCTGAAGCCCACCATCTGCTCCGACCAGGACAACTACTGCGTG
ACTGTGTCTCCTAGTGCCGGCATTGGGAATCTCGTGACATTTGGCCACAGCCTGAGCAAGACCTGTTCCCCGGCCTGCCCCATCCCAGAAGGCG
TCAATGTTGGTGTGGCTTCCATGGGCATCAGCTGCTGCCAGAGCTTTCTGTGCAATTTCAGTGCGGCCGATCGCCGGCTGCGGGCAAGCGTCAC
CCTGCTGGGTGCCGGGCTGCTGCTGAGCCTGCTGCCGGCCCTGCTGCGGTTTGGCCCCTGACCGCCCAGACCCTGTCCCCCGATCCCCCAGCTC
AGGAAGGAAAGCCCAGCCCTTTCTGGATCCCACAGTGTATGGGAGCCCCTGACTCCTCACGTGCCTGATCTGTGCCCTTGGTCCCAGGTCAGGC
CCACCCCCTGCACCTCCACCTGCCCCAGCCCCTGCCTCTGCCCAAGTGGGCCAGCTGCCCTCACTTCTGGGGTGGATGATGTGACCTTCCTTGG
GGGACTGCGGAAGGGACGAGGGTTCCCTGGAGTCTTACGGTCCAACATCAGACCAAGTCCCATGGACATGCTGACAGGGTCCCCACGGAGACCG
TGTCAGTAGGGATGTGTGCCTGGCTGTGTACGTGGGTGTGCAGTGCACGTCAGAGCACGTGGCGGCTTCTCGGGGCCATCTTTGGGGAGGGAGG
TGTGCCAGCAGCCTGGAGAGCCTCAGTCCCTGTAGCCCCCTGCCCTGGCACAGCTGCATGCACTTCAAGGGCAGCCTTTGGGGGTTGGGGTTTC
TGCCACTTCCGGGTCTAGGCCCTGCCCAAATCCAGCCAGTCCTGCCCCAGCCCACCCCCACATTGGAGCCCTCCTGCTGCTTTGGTGCCTCAAA
TAAATACAGATGTCCCC
HUMAN SEQUENCE—CODING
ATGAAGATCTTCTTGCCAGTGCTGCTGGCTGCCCTTCTGGGTGTGGAGCGAGCCAGCTCGCTGATGTGCTTCTCCTGCTTGAACCACAAGAGCA
ATCTGTACTGCCTGAAGCCGACCATCTGCTCCGACCAGGACAACTACTGCGTGACTGTGTCTGCTAGTGCCGGCATTGGGAATCTCGTGACATT
TGGCCACAGCCTGAGCAAGACCTCTTCCCCGGCCTGCCCCATCCCAGAAGGCGTCAATGTTGGTGTGGCTTCCATGGGCATCAGCTGCTGCCAG
AGCTTTCTGTGCAATTTCAGTGCGGCCGATGGCGGGCTGCGGGCAAGCGTCACCCTGCTGGGTGCCGGGCTGCTGCTGAGCCTGCTGCCGGCCC
TGCTGCGGTTTGGCCCCTGA

TABLE 12

MOUSE NOMENCLATURE
ICSGNM Rasa2
Celera mCG21645
HUMAN NOMENCLATURE
HGNC RASA2
Calera hCG16062
MOUSE SEQUENCE—GENOMIC
TGGAGCTCTGATGCTGAGGGCCCGCATGAAGGCCAGCTAGGTATAAAAATGCATCTGAAGTCCCAGACGGTTGGAGGCAGAGACGGGGAAACCA
CGAGACAAGCTTGCTACCTAGACTAGCCAAACTGGCAAGCTCCAGCTTCAAGATCTTGCCTCAGTAACTTAAGTGAAGGATAGAAGACAAGGAG
ACCTGTAACCCCCTAACCTCCACACAAATGCACACAGCTACAACAATACATGTTTGGGCCCGAAAAATGACATGCAGGTACCTGTGGTTGTCAA
GACTCAGTGACATTTTGTGCTGACTTGGAGTCAAGAGTTTCCCTCTGTTCTGGTGAACTGAGCACCTGGGTTCAGGCCATCTAACACCTACCTT
TCTCAATGGGATGACGTCAGGCCTCACAAGATACCCCTGAAAGCGCAACCATTTGTCTGCCTTCCGCAGGAACAGTAAGTTATTCAGATTCCAG
GCTCTAATGGTCCCTAGGAATGAGAAAGTTCCCTGAACCTAGTGCCTTCTGGACGGAGTGTGAAATCTGTATGTATACAGGAGTGGCCTTTGGA
TTTGATGTAGGATGTACTTTGCCACCCTGATTCAGCCTAATCAACTTCTTGTGTCTCAGCTTTCCTAGGTTTTTCTATTAGTAAAAAACCAGAG
ATCATCTCTGAACTACCCACCATTGCACTTTGAGCGTCCTCAAAGACCCCCCTTTGGTGGTGCTGTGGTTTCTTCTGTTGTCTGGGGTGATAAA
CAGCAAGGTGACCCTCCCCTCTCCACTAGCCTCCAACAGAACCACACTGTAGCTGCCTCCTCAATTCCCAGTGTGCATGGAGGACACCTGCTCA
TGATTTGTAAGAGTCTCAGCCCTACTCTGCGGCTAGTGTTACACATGTTGCTACTCTCTTGTAAAAACATAAGAGATGTCTCTGGAGTGTGGAA
GTCTGTAGGAATCCCAAGGTGAGAGGCTGAGGCTCTTGCTCTAGGTAACAGGAAAGGTAGACAAGAGAGCAGAAAAGACATGTTCCCCTAAGGA
CTGAGCTGGTGGGCTAAGGAAGTACAGAAGCTGAAGGGTAAACTGAGGAATGTGGGCGATGCATGTTTGCAACAGGCAACCAGCATCACTGGTG
CATGGGGGAGGGGGACTGCTAAGAGCACTGTTTGAGTCTGAGCAGCAGTGACATGCCATCTATGGGGCATTCTCTATCTTCCCCCTGGACCCTG
ACTCCCCCAGAGAAGGTGGACACCTCAGTGAGAACAAAAAGAGGCCACAAAAAGGGCAAGAAACTCAGCACTGTCCTCCTTCAACTCTGCTGTT
AGCCTCCCAGATGGTGACAGGCCTGATACAAAGGAGAATAGAAGCCATAGCTTTGACTGTTCTCCTCAAAATGATGCTTAAATTGTTGAAATGA
TATTTCGGGGGCACCTAAAAGAGGCTGAAAGGGTGCTGGAAATGGTACCATGCACTTAGAGCTAGAAAAGAAGTAACATCGGGAGAAGAAGGCG
AATAAAATTGTTTTGTTGCCTCTTTGGAGCCCTGCATACCCTTGGCTACTCCAGAGTTGGGGCTCTTCACTCAAGGGTCCGCCCCGCCCCGCCC
CGCCCCGCCCTGCTAGCATGTCCTCTTTCCTGGAGGCCTCTTCAGTCTCCAGATTGGTGTATCTACTTCTCTACTCAAATACTTCTCTTTCTGC
TGCTCTGGGGCTAAACCCCCATTCAGCCCTTTCACATCAAAAATGCCAAGGAAGTGGCTCAGAAATTGTGCATGCCATCCAACATGTCTGTTTG
CCAGCACTGACCTCCCCCAGTCCTCACCACTGGAAAACTTCCCAGTGGGCAATGGGGCTTCTGAAGATGCTAACAAAGAGGACCGACTGGGACT
GTGCTCTGCGGGCAGGGTGCATTGGCTCAAATCCTAGAGAGTCTGTGTGGGTCAGCTTTGCTCAGAGACAGCATGAGAAGAACAGTAACAAATG
CTGACGTTAGCTATGAAGTGTTGTCGCGTCTAGTGGAGCATGTAGCATCTACCTGCAGAGGCCGCCTCGAGGAGCTTTGTCAGTATTTTACGGC
CAGCACACTGCCTGTGCTTTGTAGAGAGAGAAGAAACTCAGCAAAGAATTCCCCTTGTGCCTGAATCCTTGACTCTTAGCCCTCATTGCCCTTC
CATTCCCAGCTTTGTTTCATGAGGGACTTTGGGTCGGCTCTTTAGCACCCTTCCCCCCAGTTACGTTTTATTTGCTTGCATGTGTGTGCCTGGA
GGCCGAAGAGGCGTTGGATCCCCTAGAACTGGAGTTACAGACCATCATGAGTCGTCATGTGGGAGCTGGGAATTGAACCTCAGTCCTCTGGAAG
AGCAGCCGGTGGCTCTTAACCACTGAGCCGTCTCTCCAGCCTCAGCCAGTTCTTCGTGAAGTTATACTCTCTCTTGGCTGAGACTTGAGAAGAT
TTTGAAAGTATCATTTAACCAACTACCATTCTAATGTATTCTCGGGGCACTAAGGAGAGGAGGGGGCACCGGAACTTTCTCTGAGCAGAGCCAC
GTGGGTCCACATTTCCTTGACTCCTGGAGGTGCCCATGCGCATCTGTACTAGCCAGGTTAGTGACAGGAACACTGGGAGGCCCTAGCAGTATCT
TAAGAGTGCGGGACTCCATTAGACCATGTCCTGAAGGTCCCATGGCTGCTGCGTTGGCAGGGGCTGCCTGGCCTCAGGTACTCTAGCCTGGCAC
TTAACTACTGAATAACTCATGGGTGGCTCTGGGATGCTGGATGAGTGGATAGCGCTGCCCAGACAAATGAAGAAATGTCCTGGCACAGAGTCCA
TGAACCAACTGCAGAGTTGCTCAACGGTTCCACCCTTTCGTGGCTGTTGTTTTATCAGTGAAAAGTGACAAGAACAGTGGACTGGAGGAGGGGT
GAACGCACCATCATAATTCAAAAGGAAGGGAGCCTCGGGACACCTGAAGTCACTCTGGCCAAAGGGCATATGCTGCCTTATGCTCCCGCAGCCG
GGCCTTTAACCGCTTCCTCCAGGAGCTAAACTTCTTTGAAAGTATTTTAAACAGATGAGCTGGAGCCTTCGGGATAAAAAAAAAAAAATGTCCA
CATATTTTACCAGTTTTGTCATGGCCTTGTTTGGTTCTGAAGTAAAATCTTAAAAGCTCTCTGAGTCTTTCATCAACTTCCCCATGTCCAGCTT
TGCTAAGAAGAAAACTGCTGCCTGGACTTAGAGGAAGAGCGAATAACTAATTCACCAACGTATTAAGATGGGATGAATGGGGGCACCAGATCAT
CACTGATGAAAGCAACTTGCCTCCCAGCCAGTCTAGGGGAAATGATTCTTCCTCATTACTGCACAGATATACGAACATGACCTACATTTAACTA
CTAAAAAAGCACAAACACATCTCCTGACTTATCAAAGAAACACCAGTAACCTGGGGGGCCCAGGGCGTCAGCCAACCCATTTGAGCCCAGCAGC
CAGGAGCCAGGAGCCAGTGGCTCTCTGTCAATTCCAGGCCAGCCAGCCCTACAGAATGAGACCCTGTCTCAGAAGTAAAATAAAACAGAGGCTG
GAGAAATGGCTAAGCCATTAAGAGCGTTTGTTCTTGCAAAGGACCTGAGCTTGGTTCGCAGTACACTAATGGAGGCTAGCTAACAACTATCTAT
AGATTTAGTTCCAGGGGACCCAATGCCCTCTTCTGACCTCTTCTGGGTGCCAGGTACTCGTGTGGTGCGCACATACACACATACATGCAAGCAA
GCAAAACATCCACTAGACACATAAAAAATATTTAAATGAAAGTAAATATTAAAATATAATAAAATTAAAAACTTCGGACCAAAAGATAGCTCTT
ACAAGTCAGTTAGAGGCTAGTAATAGGATGTATTAGTATTCAGAAATAGCATGGAACTGACCAGCTCTAGAAATAGTTTCTAGCATCAGGATGT
ATTCATTTCATTAACAGAAACGCCAATTTTCACACATCGCACCTTGAATGAGCTGACTTGTGGTTTGAAACAAATGAATGGGCTTAAAACTAGC
TAAGAAGTCAGAAAAGACAGGGCAGTGAATGACTGCTCATCAGAATAGAGAAGAGGTGGGGAGAGATAGGTTTTAAAGACATTCATTAATAATA
TGGGGAAGGAAGTGAACTGCAAGATCGTGTCTGCTGCAATATTGTTCCCGTTACCAAACACAGTGAGGAGAGGAACTCTAGACGAATTTAAAAC
CCAGCGCACAAAGCCATAGACAAAATGAGATGGAAAAGCAGTCTTGCAGCTTGCATGCAGACCTTAGTCTCTAAAACTCGATTCCTGAATGCTT
TATATTAAGGAACAAAGACTAGTGAAGATTTCTGCAGCACCAGTTGAAAAAAATGTCATTCCTAAAGTGCTCCTTCAGAAATGAAGTAATTAGA
AGTGACTTGGGATGAAAAGGAAGAGGAGCTGGAGGGCCTTAGTGGAACAATGTGATCATTTCAAACTCCACTACAGGAAGATGTCCTGGGAGGG
AAAATTAATGTTACTTCAAATAAAATTGTTTTTATATGTTTTAGTAACAAACTAGTAACAGAGTTCATGAAAAACAAGGCAACTTAGAAGAACC
TACTGTAAGCAACAGTATTTTTTCAAAGAAAAAAACAAAACAAAACAAAAAAACAAAAGAAAAGCTAGCTTTGGAAGTCTACCTGGAGGATGTC
ACAAAAGTTTTCAGGTCAGACGGCTTCCATTTGGACGACTATGATAACAAATTACAAGTGAGAAATTTCACAAAAGGGAAGCGCCACGTCTGCA
GAGCAGAGGAAGTTAAGACATCACTCATTCATGCGTGAGCACACTGAAATTGTTAAAGAATATCGAGTGCAGGAGATAAAACCCCTCGTCGACC
ACCTCTCCCTCCATGGGAGGGGGTGGGGAGGAGAGCTATGCAAAGCTCCCTCTCTCCTTGTGCTAGGAGGGTTTCATATAATAGAAGTGATCAG
TACTATACAATGACTCACGTGAGAGCCACTGCCCAGGAGAGAAAGGAGAGGAAAATGAGGCAACCCTCGGCCAGACTTTTTTCTCTTCTCTTCT
CTTCTCTTCTCTTCTCTTCTCTTCTCTTCTCTTCTCTTCTCTTCTCATTTTCTTCTTCTTCCTTTGTTTCTTTGTTTCCTTCTCTCTTTCTTTC
TTTCTTTCTTTCTTTCTTCCTTTCTTTCTTTCTTTCTTTCTTGGAGACAGGATAGCTCTGCCTGGAATGGAAGTTGATATATAGACCAGGCTGG
CCTTGAACTCACAGATATTGGCTTGCAACTGCCTCCTAAATGCTGCAATTAAAAGCATGTCTCTCCATGCCTGGTTTAGGAACCCATTCTTAAC
AAGTTGTTGGGGAAAGTCGCTCAAAGTGACTGAGGTAGGAAACGTTCGACACAGGCTGGGTCCAATCTAAGCCCCGAACACCTGGCGGAGGAAT
GTGAACTTCATTCATTAGTCATCTGTAGCTGGCTTCCTGACCTGCACTCCTCGATTGCTTGGTGACTGATACAATGAATGAAGCCATTCATAAG
GTTTTTATTGTTGTTGTTGTTGTTCTTGTTCTTGTTGGCTTGGTTTGTTTTCCACAAACCAAATAAGCTTCCTGGGTTTTGCTTTAGATTAAGA
TTCATTGGTGCAGAACAGGAATGAACGAGGTGGGAGACTTATCACATGCGTTCATGTCAAGAGCTTGATGTGGCATTTACTGAACTAATCCTAC
TAGATACGTCACAGACATGTTCTCAACACTTACTTAATACTATTGATAATCCTTCAGAGAAGGAACACCCAGTGTAACATTTCAGTTCTGCATC
AGAGAAGCACATGCACATGTTTAAAGCCATACGTTAGCTCTGTTCTTAGATGCTACTCAGAAGCCTCCTTTTGCAGTGGCCAGAGGTGGATGCA
GAGATTCACAACGGGCCAGAGAGCTGAGAATCTGGGACTGAGAAATGCTCAGCCCTAGATGAGACATCCAACCACTTCTGAGGTCCAGAGGACT
TCAAGGAGGGATCGGCCAGGAAGGAGGATGGGGAGGAGAGCTATGCAAACCTCCCTTCTGGACTTGACACGGCCTTTGTCCTCATGAACTCCAA
GCAGCTGTGCTTACCTACACACGATTAACCGGGTCAACATTCCAGCCCCGCTGTGGGAGGGGCTCGTGAGCACCTTCCCCTCTCTGAGGAGCTG
TGTAGGTAGTTAATGAATGGCTGCTGGGGTAAGGGGAATCATTTTCTTGAGGCGTGCAACCACTGTTCAGTTGCTCCAATAAATAAGCCTTGAT
CCACAGCCCTAATTACAGTCTATGGCATGATTGGGCACTTGTGCAAAAACACACACACAGAGTCACAGACAATTCTCTCTCTCTACACACACAC
ACACACACACACACACACACACACACACACACACACACACACACAAGCATCAGCACTTTTGGGGGGGGAAAGAAGGATCTCAGTAGGAGTGGGA
GAGGAATAAGGGGGTGAATAGGATCAAAATATAGTCTGTGTGTGTATGTGTGTGTGTGTGCATGTGGTCCTCACAGAATACATTTTAAGAAAAT
ATGTTTAAAGACTAATTTATTAGGTATCTTATGTATGTGGGTACTTTGTCTGCATGTATATATCTAAACATCAGAGAGGGCATCAGATCCTATC
ATAGATGGTTATGAGCAACCATGTGGGTGCTGGAAGTTGAACTCGGGAACACCAGCCAGTTTAACCACAGAGCCACCTCTCCAACCCCAAGTGG
GGGAAAAAGTCCTTTTACAAAGCTTGAATATCACTCCTAAACATCACAGCTGTAAAAGGATAGGATAAGGTTTCCTATAGCATCTACAAAGCCT
TGTCTTTGAAAAAAATTGTATTGTGGACACTCTTCAGATGAAGTCTTGTTTTTATTTTTAAAGAATTGCCATGACAGAATGGAAAGGAGAAGAG
AGAGAGAGAAAAAAAGAAGTTTCATCAGAGGGATGTGACAACCAGACACACTGTGAAGAGAAGGTCTAGGGAAATCAGTCTCCAGATGAAGGAG
AGTAGAGGAGGGAAAGGCCAACCGAAACTAACGATTATGAAAAGGTCACAAGTAAACCAGATACTTCATATGATAATTTAAAAATTGAATTAAA
TAAAAGGAGTTTGATGGAGGTACCCTGTGAAAGTAGATAAAACTTGCGCAGAGAAGCTAAAGGTCATCAAATGCAAACCCTGTGCTGGGAGTGG
ACCATTTCTGTTGTTGGTCCCACAGCCCCACCACCCTTCCAAGCAATATAAGCAATCACCACTCTCCTTGGTAGCCCACCAGAACTAGACTTGT
GAAGCCGAAGATACATGTGCTGAAGAGACCACAGGGCATACAGACATCAAGGTGACGCAGAGAAGGCGCTTCCTCCCAGCTAGCTAACCCCTCA
TCATGCCAGAAGGTGCTATTCAGGCTGCTGGTAGAGAACTGTCATCAGTAATCTTACTCAGCTGGGGTCCCTACTTCCTACATCACTGACCTGC
CAGGCAAAGGGCACCTACTGACACACTACTGGGAAGTCTGCTATGGGGGTGACCAACCACTTCCTGACTGCAACTCCTTCAGACGAAATTCACA
TCTGGTACTATAAACCTGTTAAAAAGCCTATGGCTGGGGAAGTCACAGCCCCAGTAGGGCAGGTACAGATGTTCTTCTGCTAAACAGATGTGAT
GTGTGCACCAAGTTGCTTTCCAATAATTGTGCTTGTGCATAGGAATTCGTGAGCATGTCATGTTTGCTTCGAGAAGTCCACCCCCTCATGGGCA
GTATTAAACGCAGAGATTCATAACTAGTCACTGAGAATAAAAAACTGGATGCTCACCCTTAAATAAGACCTGTATATCACCCCCCCTCGGAGGA
ACGTTGAGGAAAAGATGGGAGAAAGACTGTAAGAGTCAGAGGATAGAGTGGAGTGTGGTGGAATTTTCTTGGCATGACATCACATGGCTGTCAC
ATTTTTTGTACAAAGATATTTCTATTTGTATGTGTATGAGTGTTTACTTGCATGTATGTATACCATAATGAGTGCTTAGTCTCCCTGAAGGTCA
GAAGGAGTAGGATCCCCTGGAACTAGAGTTACAGACCATTGTGAGTTGCCACTTGGGTGCTGGGAACTCTTCTCTAAGTGCTCTAAACCCTTCA
GCCATCTCTTCAGCCATTCTACCTGACAGCAGCTGTGGTTGTCTGTACTATACAAGATCCGTGCAATACTGGGAGCCAACATCCTGTGGTAGGG
TGCAGGAGGATTTACAAGGATTTTTCACAGTTGATGGTTGCCGGAAGAGACATTTTCTTCAGTAGTACAGCCACTGTTAAGTTTCCTGTGCTCC
TGTAATTAATCTCTCGTCTATGCTTCTGGAAACAACTCTCATAAACTCATTGAACTAAAAAAAAAAAAAAAAGTCTTGCTGGTGTGATGACTCA
GTGATTAAAGGTTCTTAATACCAAACCAGATCGCCGGGTTAATCCCTAGGAAACCCACGGTGAAAGGAGAACCACCTCCCAACAGCCATCCACT
GACCTTCCAGGGGATTGGAGGGGAAGAAAGCCAGCTGGGAGGAGAGGAAGGGGGTGCAGGGGATGTGGACGGCGATGAAGAGGGTATTGAATAC
TATGCATACATTAAACATGTGTATGAAAACGGTATCACACCATGACTGTGTATAAGTAAGACATACCAATAAAGCATTAAACGTTTTAAAATAG
AAAAAGCCAGTCTCTGGCCTGTCGTGTGTGAGCTCTGATTTGGAAAGAAAGGGAAATACCATTTGGAGTTTCTGTTTGAAACAACAAAATGCCT
GGAAGGAGAGGTAAGCTCACTTTCTCCAAGGCTGTATGGTACAGGACAAGGGGCACCCTCATGCATCACCAGCTGGACTGGAAGTATTGCTCGG
TAGCCTAAGACATCCAGCAAGCTTAGGTCACTCCGTCTGCCTGACTGGCCTTGGCACAAGTGGCTTCTGGGCTTGCTTGTCGTCTGTTCACTCT
TCCCAGGCAGGCTCTCCTTGACTCTGTTTCCACAACTTGCTTCTTCTCTCTGTGTGCCCTTCCAGGTTGAGGTGCTCATGGTCTTGGCGCCTTC
TGCCCCGTCTTTTTGCCCTAGGGCCATTCCTATATGTCTGAAAACATTCCTTGACTGGCTGACTCTAGCACCTGGCAAAGAACAACCCTGCTCT
ATTAAATCAGTTTCTTCCTTCTTAGGGCAGCGCAGTTAAACCTCAGGACCAGGAAATCATCCCTCTCCTCACATACCAGGGTGTTATCCACACA
GTTTACTCACTGTAAATAGTTCCCTTAAGATAGTGGCAACAACTAGTTGAAGATTTACAAACTTCCCAGGGCCAATCTACTTTCTAAAAAGCCT
GCTTCTGGGAGTCGGAAAGAGGTCACCGTTCCGATTCTGAAATGCAAAACTGGTGATAAACTCAAAATTTCTCTGGGTCTCAGACAGGACTGGA
CGGAGTCTGTGGTCCTTTTCTTGACCCAACTTTTTCCAGAGCAATGTTCCCAGAGCAAACCTTGAGTATGTGGTTTGTCCCCCACGTGCCGCAC
TGCGGGAGCATCTAGACACCGCTGGAAGAGTCTCTAATACATCTCTTGACGTTTGCCAAAATGCCAAACTTTTTCCTGGAACCTGGAGTCATTC
TGACTGGGGACTTTTCTTTTCCTTGACCATCTGGGGACCTAGAGGTCCGTTCGTGGGGGACGTCCAATTCTCACGGTTCTGGGGATCACAGCAG
GGCCAGGGTTTTCCTCCTTGTGGGGCTTTCAGAGCGCCTCTCGCGACTTGCCCAGGGAGGCAAGGCGAGGCGGATGCAGGGCGGGCACGGCGCG
AGGGGCGGGGCGAGTCAGCCCGAGGCCGACGCGGGGCTACAGCGAGGCTCCATCCGCCCTAGGCCTCGCTCCGGCTCCGCCTTGCCCGGCTACG
CAGGCGGTAGGGCTGGGGTAGGCGCCGGGCGGCACCATGGCGGCGGCAGCGCCTGCTGCGGCGGCTTCTCCCGAGGCGCCGGCCGTCTCGGGCA
GCGCAGACCCGGAGACGGGAGACGAGGACAGTCGTGAGGTCCGAGTGCTGCAGAGCCTGCGCGGCAGGATCTGTAAGCGGGGCCGGGGCTGAGG
GGACCCCCGGGGCGGCGCGGAGGGTGGGCGGCGGNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNCGGGCGAGCGGGGCGCC
CCGGGAGCTCGGGCAGGACACTTGTGCCCGCCCCACCTTGCCGCGGCGGGGACTTGAATCGAGAGCCGGCGTGACTGACGGTGACTTGGGGCAG
GATCTAGTGGCTTCCTGTGAGCAGGTCTCAAAACTCAGTCATCCAGTCCGCGCTCAACCCACTTGATTCTCTCCCGTCCATTTGTGAGAGCCGC
ACCGATGAGTGTAGACTCTTACTCAGAGACGGGCCAGTTCGTTTGTCATTCCTATCCTTCCTCTTTAAAACGACCCTTTGATATTAGATCGACT
TCCCCCAGCTTATTGAGATGGTCCCTTTTCACTGTATGACTGGCGAATCAGTACAGTTTATTGTTACTTTAGTTCAAAACCTTTTTTAATTCAG
ATTATTTTATCCCCAGAATGAAATCGGTCTCCTAAATAATGCCTCGCTATAAGAGATATTTTATATGTTGAAAATTCAATTGTGTTAGAATGGC
TGATACTGCTCTTTCTTTGGCAGAGTATAAACTTTAATTCTTAAATCTCGATGCTTCTTTTGGATTCTCCCACTTGAGTTTTTGATGTTACTCC
CCTTTCAAGGATTTGGAATTTTATTAAAACCCCAGGAGCAAGCATGTTAAATGTTACTTTTCAATTCCCTGACGTCATCCTGGGACTCCCAATT
CAGACACTTTTAAGTATTAAAACTTGGGAAATTACACCAGAGCAGTGGGTGTTTTGTGCGTCTTAAGTTCCCAGTATTTTAAGTTACTTTCTTA
GGGTGTAAACTTTTCACAAATTAGCTTTCTTACAGTTAATATTTGCATGTATGTAATGTTTGGGAAAAGCCAAATACACTAGAATTTTCTTCCC
CTACTAATTAGGTTTTTCAAATTTTTACTACTTTATATTTTTCTCTGACTTGATCCTAGACACTGTTGAAGCCTTGCGAATCTATAAAAGCAGA
TTGTGTTGTCGGAAGACAGTGTGCTCTGTTGGAGTTGAGATTTAGGGTTGGGCTGTTTTTAGGACTAACTTACCTTCTGCTTTGACCTGATGCA
TAGCAAAGGTTCTGTGAACAGTGTGGTAAAAATATCACTCCAAGTGAAATTCGCGTCTGCAGATTAAAACACACACACACACACACACACACAA
GCACACAAAACTTGGTTTCTGTGGCTTAGGCAATCTCACTCCCAGAAATTCACCATTAATTTCCTGGGAATATTTTTATTTTAACCCTAGTTAA
ACTAAACCACTTGTTTCTACCCTAGACAGTTTATTTATTTTTTATTGCTCATCTTATAGCTCAGGTCATGTTGTGACTGTTTCACAAGGATACA
TACTGAAATCAAGAACACCCCTCCCATTTCTTGCCCTTCTTTTACCTGGTATGATCAAGGTTGGTTGCCCTCCGGATCCTTGTAAGGAAATGCC
CTTCTGTGTTTCCTGAAACTGACACGTGGGCTTTGTAAGGTGATGAGAAGTGGCGGAAACATTAGCACGTGCTGAAGTCACAGAAATGGGATGA
TGGCAGTTTGACAATGAAGAATCTAAGCAGTTTGCGAGTGTTGAAGTCACCAGTCCTTTCTACCACACTGAGAAACCTTGGGGAGAACCTGGTA
GCTCCCTGTTAGCCTGGAGATGAGCTGTTAAGCTCAGAACTGCTCTTTGGGAGCCTCTCATCTGATCCACCTGAGAGGAAGCACTCCTGGGTAG
TGTAGTCCGAATCATCCTTAAGAAGAGCGAGGGTCGCCCGATGTTTATATTGTGGAAGTTCTAGAGGTTAGATAATGGTCCACACTTTCTTCAG
TGTTTCATTTCTAACTTATTTGTGTCCCATAGATTTCACAAGGTATAAATAGTATCAAGTTGACAGATCAGAAAACCAGCGGGTGGGGGACATG
GGCTCAGTTGGTAAGTGCCTGCTTAGCAAGCAGGATCACCAGAGCCCAAGTAGAAANNNNNNNNNNNNNNNNNNNNNNNNNNNNNNNNNNNNNN
NNNNNNNNNNNNNNNNNNNNNNNNNNNNNNNNNAGAGAGAGAGAGAGAGAGAGAGATCAAAACACCAGATGTTGGTCTCAGGCACCCACACCCA
TGGGCATGCACATGCACACACAGCTAGTGCACACAGTCAGTACATTAACAAGTACACACACCACACAAACACACACACACACACACACACACAC
ACACACACACACACAGAGACCATCATATAGCTGTTTTCACACAACCAGTTTAAAGAAATCTATTATCGCAGACAGTATTTTGTGTTATAGACTG
ATTAGTGCCCAGTTTCTTACGGCATTTTAGTGTCCTAGATATCCTGAGGGTTTTGAATGTTCAGCTTCCTTACCTGAATTCACGACACAAAGTT
GTTAGATATCTTAGAATTTTCATCTGAACCTTTAGTAAACAAAATTAGGAGTATTTTTGGTCTGAACTACTTTGATATAAGTAGCATACCTAAA
GGTAGTTTGTATGCACCCCCCCTCCATTATACCCTATATTCAGGTGAAAAAAAAGTTTTAGATTTATATGTGTGTGTGTGTGTTTATGTCTGTC
TGTCTGTCTTCAGGCTTGATATGGCCTCGTTTTTGGTTACCCCCTTTTCTAATTAAGTCAACTGTAAACATTTTGAAAGGCTCTACACTACCTT
ACACAGGGCTTTTCTCTCAGGGATACCAGGTTATAAAGGCACCAGCCTGCGAACATTTGTGGGAAGTGTTTGATATATTGTTCAGTGAGATCTG
TGAGACAGTGATGTGGCCCAAGAACTACTCACTTACGATTTTGACATTTTCAGACCAAGAATCACTTCCGGCTGTTTGACAAAGGAGATAGATT
AAGTACTTAAGCTTAGGAGGCCTGGTGTAGTCAGTACCTTCAGCCCCAGCCCCCTAAAAAAAAATCTGGGTCAGCCATGGGCCATCTCTTAGGA
AGGAGTTAAGTAGCAGATAAGCCTTATCTGATGCTCTTGTCCAGCCTGCACTAACGTTTCCTGGTGTAGACAGCAAATTGGAAGCAGAAAGCCC
GTGTTCTGCTTCATACTGTGTTAGGTAATATTCCTGTGACTTCAGGCAGCTGTTTACTCTTGAGCTTGTGGTTGTTTTTATCTTTCTCATCTTT
AGAGTGGACTAGTGCTTTGTCACCAGACACTTGGCTCACCTTGTTTCCTCTCCCTGAGATTGTGTCCCCCCCTCCCCTTATTTATGTCAGAGAG
CCACATATCCTTCCAATGCAATCCAAGTCTCCTCTGACCAGATTTGCCCTTCTTTCTAGCCATATGGCTTTGATGCCTAAAAAAAGAAAAACGT
AATTCATGACCATTTTCTTCTTATAAAAATCATTTGTATATGTCCCCTGTTATGTTAATTTTAGAATTCATTCTTGTTTTTAATATTTGTTGCT
GCTGGTGGTGTGTGTGTCTCAGTCTCACTAGATCAGGGCAGGCCTTTAGAAAATACCCTATCTCTCAGTGTTATAGATACTTTCAGAGCACTGT
GTATCTGGATAAGAAAGAATTTCCATATCTCTGGACAGATTAGCAGGGTTAGCTTTAGTAAAGGAAAGCTTCCTGAGTCTCTCTGGAAGTTCTT
TATTCTTTTTCTTGAAAAGTTGTAATGTCTGCTAATCTTAGGTTTGAACTGAGGCCAAACTTGCCTCCTTAGGAAAAGGATTACATTTTTTCTA
AATGCTGGCTTATTAATTAGAATATTAATTGTTTCCTGACTGTGTCCATTAGTTTTGTTACTAATTGGTCCCCAGAGCTTGGAAAAGAAAATAT
TAAACTCATACTTTACCTAGAATTGTTTTTATTTGACATGACAGGTTTAAATGTTTTCGTACTAAGATGTAAATCAAGATGTTTTATTGACTAC
CCAAGGGTTGAGAGGAAGAGATTTAAAAGGGATTATTCATGCTAATAATTCATTATTATCAGATGTTTGAAATGAGATGATGAATACTTAGTAG
AGGAATTTAAAAGGGTTTACTCATGCTAATAATTCATTATTGTCAGATGTTTGAAATGAGATGATGAGTTCTTTAGGACGGAAGAAGAATCCAT
CTTTGAGATGGATAGAAACAGAGGAAAAAGCGACCTTTGTAAAAGTTCACAGAGCATTCAGCTAATCATTTGCTCTGTGCAGGACAGGAATAAA
CCTCAGGGAGTTCATAGAGTTTTTGTAACTGAGAACTCTTTGAATAGCCCTGGACTGACTGGGAAGTAATGTAGCTAGAATAAAACCACCATTT
TTATCCTTTAATTATAAACCCTAATCCTATTTCCTTGTTAACGTGCTTTCTGAAATTATTTCATAATATCAGATTCTGTGCTTTACCTACTGAT
ATATTTATACCCTTTTATACCCTAGTAACAGTTTTGTTGTATAGTCATGTGTTATTGTTAAATTAGGAACATTTTGTGTATGTTAAATTTTTAT
GACATCTAGTTTTGTGTAATGTTTGTTTTTTGATTTTTTTTGTTTTGTTTTTCGACACAGGGTTTCTCTGTGTAGCCCTGGCTGTCCTGGAACT
CACTCTGTAGACCAGGCTGGCCTCAAACTCAGAACTCGCCTGCCTCTGCCTCCCAAGTGCTGGGATTAAAGGCGTGCGCCACCACGCCCAGCTT
TGTGTGATGTTTTAATCATCAAAAATCTCTAGCTAGATTGGCTTCATTCTCTTTGTGTCTGTGTGTGTCTGTGTGTGTGTGTGTGTGTGTGCGC
GCGCGCAGGTACATATGCATGTGTGTATATGTAGATGCCAGAGGATGATCTCAGACATTGTCCTCAGGAAACACTGTCCACCTCTCTTATGGCT
TGGAGCCCTACTAATTAGTCTAGACTGGCTGGCCAGCAAACTCCATGGATCCCTCTGTCTCTACCATCTCTGTGCTGGGTTACATGCAGGACCC
TTCACACCCAGCATTTTTGCCTAGTTTCCAGGGATTAAACTCAGGTCCTCAGGTTTGAGGCAACCAAACATGACTTACTGACTGATCTCTCCCC
CTAGCCCCAAAATGGCCCATTATAAGGTTATATCAAATATGAGCCAGGTGGTGCTGACATAGGCCTTTAATCCCAGCACTTGGGAGGCAGAGAC
AGGCAGATATCTGAGTTCAAGGCCAGCCTGGTCTACAAACCAAGTTCTAGTACAGCTAGGGTTACACAGAGAAACAGAGTCTCCATTGCCACCC
CCACCAAAGGAAAGAAGAAAACATTTTTATTATTGAGTGTGGTTTCTTTTTCTTTTTTTTCTTTTATTGGGTATTTTCTTTTCTTTTTTTTAAA
AAGATTTATTTATTATTTATATGAGTACACTACAGCTGTCTTCACTACATTTCAGAAAAGTGCATCGGATCCCATTACAGATGGTTGTGAGCCA
CCATGTGGTTGCTGGGAATTGAACTCAGGAACTCTGGAAGAGCAGTCAGTGCTCTTAACCACTGAGCCATCTCTCCAGCCCAAGCCTTCTCTCC
AGCCCGGGTATTTTCTTTATTTACATTTCAATTGTTACCCTGTTCCTCCCCCAAATGTTATCATCCCCCCTCCCTCTGCTTCTATGAGGGTGTT
CCCCCACCCACCACTCCTGTCTCCCCACCCTCGAATTCCCCTCCACTGGGGCATCAAGCCTTCACACGACCAAGCGCCTCTCCTCCCACTGAAG
ACAGAGAAGGCCATCCTCTGCTACATATGTAACTGAAGCCATAGGTCCTCCXEGTGTACTCCTTGGTTGGTAGTTTAGTCCCTGGGAGCTGGGG
GGGGGGGGGATATTGTTCTTCCTATGGAGTTGCAAACCCCTTCAGTTCTCTCAGTCCTTTCTCTAACTCCTCCATTGGGGACCCCATGCTCAGT
CCAATGGTTGGCTGTATTTGTCAGGCTCTGGCAGAGCCTCTCAGGAGCCTGCCATATCAGGCTCCTGTCAGCATGCCTCTTGGCTTCCACAATA
GTGTCTGCGTTTGGTGACTGTATATGGGATGCATCCCCAGGTCGGGCAGTCTCTGGATGGCCTTTCCTTCACTCTCTGCTCCACACTTTGTCTC
AGTATTTGCTCCCGTGAGTATTTTCTTCCCTTCTAAGAAAGATGAAGCACCCACACTTTGGTCTTCCTTCTTCTTGAGCTTCATGTGGTCTGTG
AATTGTATCTTGGGTATTCTGAGCTTTTGGGCTAATATCCACTTATCAGTGAGTGCATAGCATGTGTGTTCTTTTGTGATTGGGTTAACTTACT
TAGGATGATATTTTCTAGTCCCATCCATTTGCCTACGAATTTCATGAAGTCATGGTTTTTAGCTGAGTAATACTCCATTGTGTAAATGTACCAC
ATTTTCTGTATCCATTCCTCTGTTCTTTCCAGCTTCTGGCTATTATAAATAAGGCTGCTATGAACATAGAGGATCATGTGTCCTTGTTATACAC
TGGAGCATCTTCTGGGTATATGCCCAGGAGTAGTAGAGCCCAGTCCTCAGGTAATACTATGTCCAATTTTGTGAGGAACCACCAGACTGATTTC
CAGAGTGGTTGTACCAGCTTACAATCCCACTAACAATGGAGAAGTGTTCCTCACATATGTACAGCTTTCCTCTTTCTCCACATCCACACCAGCA
GCTGCTGTCACCTGAGTTTCTGATCTTAGCCATTCTAACTGGTGTAAGGTGAAATCTCAGGGTTGTTTTGATTTGCATTTCCCTGATGACTAAG
GATGTTGAATATTTCTTTACCTGCCATTTTAGCCATTCGATATTCTCAGTTGACAATTCTTTATTTAGCTCTCTTCCCCATTTTCTAATAGGGT
TATTTGGTTCTCTGGAGTCTCACTTCTTGAGTTCTTTGTATATATTGGATATTAGCCCTCCATCGGATGTAGGGTTGGTAAAGATCTTTTCCCA
ATCTGTTGGTTGCCATTTACTTCTGTTGACTGTATCCTTTGCCTTACAGAAGCTTCACAATTTTATGAGGTCCCATTTGTCGATTCTTGATCTT
AGAGCATAAGCCATTCGTGTTCTGTTCAGGAAATTTTCCCCTGTGCCCATGTGAGATCTCAGAAGAAGATGGAAAGATCTTTCATGCTCATGGA
TTGGCAGGATTAATATAGTATAAATGGCCATCTTGCTGAAAGCAAGCTACAGATTCAACGAAATCCCCATCAAAATTCCAACTCAATTCTTCAT
AGAGTTGGAAAGAGCAATTTGCAAGTTCATTTCGATTAACAGGACAGTGAAAACTATTCTCAACAGTAAAAGAACTGGGGGAATAACCATCTCT
GACTTCAAACTGTATTACACAGCAATAGTGATAAAAACTGTATGCTACTGGTGTACAGAGACAAGCAGGAAGATCAAATGAATAAAATTGAAGA
CCCAGAAATGAACCCACACACCTATGGTTACTTGATCTTTGACAAAGGAGCTAAAACAATCTAGTGCAAAAAAGACAGCATTTTCAACAAATGG
TGCTGTCTCAACTGGAGGTCATCATGCAAAAGAATGCAAATTGATCCATTCATATCTCCTTGTACAAAGCTGAAGTCCAAGTGGATCAAGGACC
TCCACATAAAACCAGATACACTGAAACTAATAGAAGAGAAAGTGGGGAAGAGCCTCGAACACTTGAGTGTGGTTTCTTATAGTGCTATTAATGA
TAGACTAACCGTGCATATTGAGGAGCCCCTACTTTACCTCAAAAATGAGGACTTGGGTGTGTCTTTCAGAAGATACCCCTCAAATTATTTAATA
GTTGCTTTACGAAGTGCACCATTTCTCTTAAGATAATACATCAGAGCCCTTTGCTTTAGACTTGAGTTTGTTTAATATACTTGTTTTGTATTTA
ATGCAGTACTTTTAAGAAATTCGCCTAAGATTTATTAAGGTTGTTTGAATCCTAAACATTTTCCAGTGGGCAGTAGTTAATTCAGTAGTTGTAT
ATGATACATAGTTGAGTATCTGATAGGGTATATCAACCCCTGGTGCTGAGAAATAGGTCTAAGCTGTGAGTCGGATGAGAAAGTCTTTACTCAG
CGTTTTACTCTGAAATTTATACTAAGCAGTAGCGTTCATTCATTCTCTGCTTAGAGAGTTAGAAAGCTTTAGAACTGGAAGGAAAAGAACACTG
TTGTTCTGTTCATGTGTACGTGAAGACTAGAGGACCCAAGTGGCACACGTTAATCACTTGTTGGACACTAGTGCACTGTTTTTTCTAGAGTGCT
GTTGATTTATTACTATGGATGTTGAAAATGCACTAGATTTAAGCTATGAAGACTGCTTTACCTCCCCGATGGTTTTTAGAGCGGTGCAGTTGAG
GTTTGTACCCATGCTCATCTGCTCCTGCTAAGAACTTAGTGACCTGATTAGGACCAGCAATGCCGTGACCTCTGTGGACATAATCTGGATGTTT
ACTGTTTGTGTCAAATCCCACCTTAACTTTTTGCAGTCCATCAGTGTCTCTTGAATACTTAAAAACGTTTGATATCCACACTCTAAATAAAATC
ATCATTTTCAGCGTCTACCAGCTTAGTTTGTATCTTTTTAGAGGTAGTTTAAGCATAAACAAATAATTCCTTGTGTATTGTAGCTTTTCCACTT
CAGAGAAATAGAAGCAAAATATGCAGTGTTCTGCATCTTTTTAAATATAACTTAAAGATGTTTTTATATCCTCACTTAAAAATAATATAGTATG
TATAATACCTCATCTCACATCATCTAGATGTATTGCATTTTAGCTAACCAGTCACCTGTTGATTCCAATTTAAGTTGTCTCTTGTCTTTGCAAT
AACAAACATTGCCACCATAAATAGCCCTGTTTATCATGTATATGTGTAAGGTGTGTGTGTGAGTGTGGATGTGTGCTACTAGGGAGAAAATTCA
GGGTCTTATTTGCACTCTACCATCAAGCCAAGCTGTAGGCCATGTGTGTGTGTGTCTGTGTGTGTGTCTGTGTGTGTGTCTGTGTCTGTATGTG
TGTCTGTGTGTCTGTGTGTATGTTTTTGGAACCACAGTTTCTCTGTCTAGTTCTAACTGCCCTAAAACTCACTCTGTAGACCTGGCTGGCTGGC
CTTGAACTCAGAGATCCACCTGTCTCTGCCTCCCACGGGCTGAGATTACCATTGCCTGGCTGCTACATGTTATTTTAAATGTGAGCTCTTGCTA
TGCAGCCCATGTTGACCCTGAATTTCGGATAATATTCTTGACTCTTGCACTGGCTGAGTGTCCTCCGATTACAGCTGTCCACCACCATGTCTGG
GCTTCATACAGATTTTTAAATTAAAGAGGTTAATAGAAAAATGAATGCAAAGACACTCCACAGAAGAAAATAAATTATCTAGTAAATATATGAA
AATGCTGGTGAAATTGACCAGAAATGAATCAGAGAAATAAAAGTATAAATACCTCAACACTTAAAAAAATGGAAACCTTGTATATTTCAGTATT
GAATGAATGCTTAAGGAAACAGATACTGTTCGTACTTTCATTGTGATTTTACGTTTATAAAACCATGTTAGAGCATTTGGTAGTATCTAATAAA
AACCTAGATGTATTTTTCTTATGCTTTTACTTTTGCCATCTCATCCTGCAGACACAAACATATTTTGTGTTGACCTTGTTTGTTTTTTAAAAAG
GCACATTGAAGTTCTAGTAACAGGAAACTGGTTAAAGTGGTTTGTTCATAAATATATTCTATAGCAGTTAAACAGAATAAACTACATCTATATT
ATGATGACCCCAAGGTGTATTATTGAGTGAAAAAAAGCAAATTGTTAGAGTAGTATGTATGTTTATTATCTGTCATCCATCTATCCGTCTGTCC
ATCTATCCCTTTATTCTGTCTCTATGATGCCCAAGGATCAAGCCTGGGACATCACAAATGCTAATTAAATTTTCTATCACTGAGCTACAAGCCA
GCCCCTTAATGTATGCTTTTAAACTCTTGGAACAGTGCGTTTTTCCCCCCTATAAATTAGATAAATTTTATTTAAAAATGTCTACAAAGATGTA
TATTGATTTTTTCCCCAATGATGGGCAACAGGAATTACTCTTCTGTAATGACTTGATTTTCAATAGGGAGTATAGTATGTATTTGAGATGTTAT
TTCAATTTTCTATTTGTACAAATGAATTTGATAGTGTCAGATATTGTGAGACTCAATTTAACTTTTGATTGAATACTATATCCAAAATCAGACT
TAGTAACATTTTAAGCATACATTGAAATGAATAATATCAAATTTTCTGTTTTCAATTTCTACTTACTAGTTTCTATATAAATGTATGTAATTGT
GTGGGAAGGTGTGTGTCTGTGTATGTGTGTGCATGTATAGTGGACTACTTCTAGGAGTTGATTCTCTTCTTCCAGCTCCACCAGGTCATCAGCA
TTAACAGCAAGTGCCTGCACCTGCTCAGTTATCTCACCAGCCCTCCTTTTATGTCTTTTCGGCAATTTTAACGATGCCTATTTAATCATGAATG
TGTGAGTGCAGGTGCCTATCGGAGCTAGGGGAGTTAGACGTCGTTCGGACCACCCTCTATGGATGCCTGCAACTGAACTCAGGTCTTCTTCAAG
AGAAACATGTGTTAGCCCCATAATTTTATTTTGAGGTTAAAGAAAATGAAACTCAGAAAAATTCAATGGATTCCTATGTACTTTGCCTGTTTGA
CCAGTTGCTTATGCTAAGAAGATTGACTTTTCGAAAAGTTAATACTTTTCAAAGGAGTGTTATATAAAGGTCCTGAATAATTTTGAACCTGTGT
ATTTGGACAGTCTTATTTATATAAAAATTGCTCAGTATATAAGCGGAATGAAGCAGTGTTTACTGAATATTTGCTGCTTTCCCGATATTGGGCT
AGGATACTTTGCTTCATTTCGTTACAGGATTGCTGTTCTTTAAGAACCAGCAGCAGAGAGGCTCTTTAGCAACACCAGATTTCAGCAGGCTTGT
TGACAAGGCTCATGTCCGTTCCCTACACCAGGTTTTCTGTCTTGTAGTGTACTGGCATGAATTCATTTTTGTTCTCAAGACATGGTGATTTCTC
TAAATTAACAGTGGCCTAATGAATAGAGATTAAAGCTAGATTGTTGTCAAACACAGTAAAAATGAGTCCAGGGATTTGTAAAACTATTCTTTGG
TTTTCCTAACCCTGCTTTTGTTGGTTTTTCTCTTCTANNNNNNNNNNNNNNNNNNNNCCCCTCCCTCCCTCCCTCGGTCCCTCTCTCCCTTCCC
CTTCCTTTCTTCCTTTCTTTCTCTACTTCCCTTGTTTTTTGACATGAAATCTCTCTCACTATGTAGTTCTGGCTGGCCTCAAACCCTCAGCCTA
GTGAGAGTGCTAGAGCTCAGGTGTGTGCCACTGATCTCTCTGTTCTTTAATGCACTTTCTTGCTTTTGAGTGGTCCCCCTAATATGTACACACC
TTTTATAAATGGGAGTTGGGTATGTGTATCTTCTTTGATAAGAGTTCCATTATCAAAGATAACACTCACATCCCATGTAAGGCCTGTCTACATG
CCCCGAGACTCTCCCAGCATCAGCTTTTAGCTTTTTGCTTGGCATTTCAGGCTCTTTAGGTTGCCTTATGAGCCTTGCTAAGTATCATGGAGCT
GTAGGGACCAATGGCCTAGCTCAGCAGAGATTACACCATAATCTCTGATTGCTTTCTCACCATGCACAGGGGCCTTTAGGGACCTTTAGGGACC
TGCTCGAAGCTGACACCAGAAGTGAGCAACGCAGACCACAGCAACATCTTCCTGCACTTGGTAACATACGTCTATAACCTATGGACATTTCCTG
GCAATACCTTGTGAAACTAGCCTTTTCACGTACAGTAGTTTCCTAACTTCTTGTTCTAGACTAAGCTTCAGTCAGAGGTGAGTAGCATGATCAC
AATATAGATAACTCGAACAGTTATTATGCATTATCTTTGATCTGAACGTGCTGCCTTGAATAAGGGAATTTTATATAGATGCTAACTTTTAAGA
ACCTCTAGCCCAGCAAGAGTACTAGTTCTTGTTTTGATTTGCTAAGTACTTACTTTCTTTTAGATATTGTGCCAAGTGCTTTACCACAGTTAAT
GTTCACAACAGTCCTAAGTATTACCTCCTTTACACAGGAAGTTGAAACTAAGGACATAACAGATTCTAAAGTACAAAGCTTATATTGAAACTCA
GATGTCTGTCTTAAAATGCACTCATTTAACTTGCTGTATTGTCACTACTACTTGTTTTAGAATTCCCTGGTGCAGAAGTTGAAATTAAAATTAG
CAGAACATTTTGAAGACTTTACTATATATTATCATATCATACCTAGGGTTCTCTGATGTATACTTAGGTAGCATTTTAATTTTATCTAAGCTTA
TCAACCTGAGTCCGTATTTTCAAATATTTTAAAGTTTTTTAAAGATTTATTTTTTTATCGTCAGTGTATGAAAGCTTTGTCTGCATGTCCGTGT
TTACATGTGTACCTCATAGCCTCAAGCAACACCATATCCCCTAGACTTGGAGTTAGCGGTTATTGTGAGCCATCATCCGGCTGTTAGGAACCAA
ACTTAGTTCTCTGCAGTAGCAACAAGTACTTTAAACTGCTGAGCCATCTCTCTTGCTCCCACAGTATATTTTTAAGTATGTATGATCCTTCTTT
GTTTTTTGTTGTTAGTGTGAATATATGGTGACGTTTACATACTGTCATAGCTCTCACTATGTAAAAGCTAAAAGCTTCTGTTTAGGTTGACATC
TCTGAGGACATCATGTTTTGTGTCACTTGAGCCAGTTTATACCAGATTCTATGAATTCTCTCTCTGTAGTTTCTCTTCAGTTTTTTCTTTCATC
TCTTTCCTTTTCATTACCTCAGTCTAACTTAGAACTCTGTAGCCTTCCATTTTAGGTTCTCTGTTGGCCTCCATCCTGTATCCTCCTGTTCTCC
CTTAATCCCCCTGCCTTAGTTTGTCATATTCACTGCTGCCTTCAAGATGCTTCTTGTTCCCACCTCCATTACCAAAGATGACATCACATCCCAT
GTAAGGCCTGTCCGTGTGGTAGGCCTTCTATTGCAACATTGATCTTCTGGTCTTAATTTCTGTATTTTTTTTTTTTTTTTTTTTTTTTTTTTTA
CAGTTGGATGATCTTTTTTTTTTTTTTTTTTCTTGTACAAAGAAAATTTATTTCTCTATGTTAACATTTTTCTTAAAATGCCTAGGCTTACTTT
TCTTTTTTTTTTTTTTTTTTTTCCCCCATTTTTTATTAGGTATTTAGCTCATTTACATTTCCAANNNNNNNNNNNNNNNNNNNNCTTTCCCCTG
TACCGGGGCACACAAAGTCTGCGTGTCCAATGGGCCTCTCTTTCCAGTGATGGCCGCCTAGGCCATCTTTTGATACATATGCAGCTAGAGTCAA
GAGCTCAGGGGTACTGGTTAGTTCATAATGTTGTTCTACCTATAGCGTTGAAGATCCCTTTAGCTCCTTGGGTACTTTCTCTAGCTCCTCCATT
GGGAGCCCTGTGATCCATCCATTAGCTGACTGTGAGCATCCACTTCTGTGTAATTTCTGTATTTTGAAAAAAAAATGCACCATTGAAAGATTTT
AAGTAAAATAACATGACATTCTTTAAAAATCTGCTATGTGTAACATATTAACTTATTAAGTAAGTGGTACCTTTTTATTATGGTTTGGCTTGAC
TTTTTCCTGCTCCAAATAGTCTTTATTATTATTATTATTATTATTATTATTATTATTATTATTATTACTTTGTTCAGGACCCTGACCTATTATG
ATTTCCTCTGAGGCTTGATCCCTTTAGGATCTATGTTTGCTCCTTTTCTTCTTTGTTTTAAAGGTATGCTTTGAAGTAGGGCTGGGTTTACCAA
GTCTCTTTCAGACAAGGATAGTCTCACAACTACTATTTTCGAAAATCTTTAAGAATAGAGTGGAAAGATAGATAATCATGCTATTGGCTTTTGA
CTATGATTTATTCATTGATGCTCTTGCTGCTGCAGATTTACTTTATAAAAAGGAACAGAATTGCTTACCTAATAGGGTTGTTGTACAAATCAAA
GGAGACATTTGAGAAAATCCTTAGTTGTGGGTCATCCAGATATTTAAGAGATAGTGTATCCAGTACTATGCTCACCTAGCACATTCTTAAACAA
CAATAAAACCCACTGATAAGTATATATACAGAATCTTCCCTAAAACATCTACATAAACACGTGTACACACACAGGTGTACAAACCACACTTATC
AAAGTAAGTGTGAGCAGCTTTGATGTAAGTATTCTAGTGCCAGGTACAATTTATATGGTAGACTAGGAAGATGGCTCAGTTGGTAAAATGTTTG
CTGCAAATCATGAGAATCTTAGTTTGATTCCTGCAACTCACATAAAAAAAAAAAAAAACCCAAATATGATGAGATGCACTTGTAATTCTCTCTT
GGGGGAGATGGCACAGGAGGACTCCTGGGACTTGTGGGTTAGTTAATCTAGGCAAACTTCTTAGCTCCACGTTCACTGCGAGGCTCTGTTCCCT
AAAAATTTACATGATAGGCTCTAAACAGAAGCATGCCCGCATGGAGTTACTGTATAATCTACACAGAGAGGACAAGCATCAAGACAAATAACGT
CGGGGCTGGAGTGGAGGCTAAGCAGTTTTATGAGCATATATACTCCTCTTAGAGGGGATGTGAGTTTAAAATTCTCTTAGAGAGGATGTGAGTT
TTTAAATTAATTTAATATAAAAACTGATAATAGCTGAGAATGGTGATAACCCAGCACTTGGGAAGAAGAGGTAGGAGGGGGAAGAGTGCAGGGT
TATCTCTGTCTGTCCAAGGCCAACGTGGAAGCTCTGTCTCAACAAAAAAGGGAAAAATTGTGTGTGTGTGTGTGTGTGTGTCTGTGTGATGAAG
AAAAGCTATATCTTATTCGTCAGCTAAAACTGCTGTCATATAATGTGAAGGTGTTTTGGCCAACTTACACTGCATCTATGAAAGAAGAAATATC
TAATGAAGAGTTTATTTTGTTCATCTCTGAAAATTTGTGGTTTCATAAAAGGGACAATGGGTGCCCCGTGGATTTCTTCTTTTTTGTTTGTTTG
TTTGTTTTGGTTTTGGGGTTTTTTGTTTGTTTGTTTGTTTCCTTTGGTTTTTCGAGACAGGGTTTCTCTGTATACCCCTGGCTGTCCTGGAACT
CACTCTGTAGACCACCAGGCTGGCCTCGAACTCAGAGATCCACCTGCCTCTGCCTCCCAAGTCCTAGGATTAAAGGCGTGACCCACTACGCCCG
GCGGATTTCTTCTTAATACATACGAAACCTGTAACTGGTACTGCTTGCTACCAGAAACTACCAAAACTCAGTGTCCTTCTGTTTTCCAGTTTTC
TAGTGAATGACTCTGATTACTCCAACTTAAATTAGTAGAACTTACAAACATAAACATAGCGGCAAGATAGGTTAGGAGAATTTCTAGCTTTAGA
GTCTCCATAATACCAGAAAGCACACAAGTAATGCTGTACCGCTAACTCACCATATCTTGTATGAGTATAAAGTTGATTCTTCTTAAGAATAGAA
CTCAGTATGCAGACTGGAGTTGCCTTGAACTCAGAGATCCATTCACAAAACTTCCTCAGTGCTAGGATTAAAGGTGTGTGCCACCATGCATGGA
GAGTCTTTTTATTTTATAATGGCATGTTTTAAAAATACCTAACAGTGCCCTTCCTCTGATGAATTAAATAGTCTATTCCAAGTTTCCTCCCATT
TAAATATGCTCTGTAGATAAAAGCCTAAATGAGGGGTAAAACAAAGACCGTTTAGCAGGAGCTGCCAAACAGAGGGTGGGTTAGTGCTAAGATA
CTAGAGAGTGGCCACATACTCTGCTATTGACACGCAGTAATTCGATTCGGAAGTGCTTAGTGTTACTAGTGTTCGTAAAGGTTCTAGGCAAAGG
AAGGTCTGAAGCTAATTGTAGAACGCTAGGGTATGAGTGAAAGAGGAGGGTTAGAGGATGGGTGATTTTGACTCAGTCATGTGAGTTATAGCCT
AAACTTGCCTGGAAGTGGTAGGACCATCTCTGGGAAAATGCAGGCTTCAAATGTGCTTCACTTTGTACTTTATGTACTTGATGGTGGAGGGTGC
GCTGGCAACACCTTCTTCTGCTTAGAGATGCAGAGAGGCGGCAGTGCTTTCGTTCGAACCATCGCCTAGGGTGCTTTATCAGTGAGCAGTAATC
TCTACTGTAGTTTGGAATTACAGTTTAAAACTCCTCATAACTGATAAACATTACTGTGATTATTTTGGAGAACTGGGTTTGATGGGATGAATTT
TCAAAACAGCTTATTTGAATGTCAACCAGTGGTAAATAGTGAAAAAAAAAAAAAAACATCAGGAAAACTACTTTTATAAAAAGTGGTGCTGGGT
GCATTTCTTACACTATCTGAGACTTGGCCTTTGATTATTTTAAGTAAATATCTGCCAATTCAGTTTCATTAAATTCTACAGACAAAGTAACCCT
TGTTTCTCTCTTGTGGGGTGGGACTGGTAGATTATTTACACATTATGCCCCATTATTTTCTAACCAGTGCTTAGCATGAATAAAACAGGGACAC
GTCCTAGGGTAATGATTTAATTTTCTAAACTCTCTTTTACATATGGAGCGTATTTATTGTACTCATATGTGTATGTATGTATATATATATATAT
ATATATATATGTATGTATGTATGTATCTATGTAACTAGGTGGTGTATTATGCACTCTATAAACTTTGGTTTTAAAACAAATCCATTGTGTTATT
CAGAGGCAAAGGAAATTCTGAACAAGTTTGACAGCAGTGAAAATAGAATTGTTAAGCAAGGTCCTATGTAGGGAGAGAGGAGGGACAGGGGAGA
GAACCTCAAACTCACAGGGATCCTCCTGCCTCTGACTCCAGTGGCTGGTATCACAGGCATGACCACCATTCCAGCCAGTTCATTTTGTTTTTGA
AATGTGGTTTCTCTCTCCCTGTTTGTTTTGTTTTGTTGTTTTGTTTTCCCTTAAGATACTTTCTCTGAATAGCTTTGGTTGTCTTGGAACTCAC
TCTGTAGACCAGCCTGGCCTCAGTCTCACAGAGATCTACCTGTCTTTGTCTCCTGAGTGCTGGGATTAAAGGCATGTACCACTACTACCTGGCT
GCAGGTTTTATCTTGTTGACGCCAGAGCTTCTGGGTGGGTCTCCTGTGTCACCTCCCATCGTCTTACTGTAGAAACACTGGAAACATTATGTGC
ACTCCAGCTTCCAGCTAGTCAGGATTGCACACAAATGTTCCCACCCACTTAGGCTTCTTACTGGCCCCTTCCTCACTGTCTTAGATTTGTACAT
TGGTATGGGATTTCAGTATACACATCAAGTAGACACTTGGGTGTCGTCCTTTTGGTACACAAATAGCTCATGAGACCTTCAAAGCTTGACTTGC
TGGGTTCTTTATTCCTTCAACAAACATATCACCAGAGTTTTCCTGTTTTACATCTTCGGCATTAGCAAGTTCTCAGACCTTGTACAGGAGCTGC
AGAAACCCTTAACAGAAGTTCACCCCATTACAAATTCTCTCAAGGAAACCAAACCTAAGACTAATTAGGGAAAAGGTCAAGTGTCATTTGAGGT
CAGGACTGAATGATAAGAATGTCCCAGCCGTGTAAAGAGTACAGAGATCCGGGAAGAAATAAACTTGATAATAAACTTGACATCGCTGATGAAT
GGAGAAAAGTGAACCAAGGGAACAACAAAAGAAGGAGTCAGAAGCGATACTTACGGTCATGCAGAGTGTGATTTAGAGATTAGACTTTATTCTC
AGACAAGTGGGATGAGCTGAGAAGTGACTGAGTTCAATCTTATGAGAAGATCATGCTTGGTCTGGATTATGGCTCAGCAATAGAGCATTCGTCT
ACTCTGCATTCATGCAGACCGCTTGGGTCTGATCCCCAGCATGGGAAAGGAGAGAAAAGATCACATCTCCTGGTGAAGAACAGGTTGGTGAAGA
GTAGCAGAAACAGAAGATACTGCCATGCTCCTCTGCAGTGATCCTGTTTGGGTGATTCTAGTGAAGGGGAGACGTAGCTGGGTTTTAGATGTTG
TCTTTTTGAAGATCTTCTGGATGTGCTAATCAGCTGGATTGTGTGAGGCAGAGGCAGGAAGACGATAAGTGAGAAGGAAGTGGTGGTAGACCAA
GGGAAGAGCTTATAGAAGAGGAAATAAGATTGGTTTTGGGGACTTGCTAAATATGAGATACCTGCCAGATATCTAGTGGTATGAATGAGGGAAG
GTCAGGCCTGCAAACACAGAACTGGGAGCTCCTAGCATTAACAGTGTGTACTGCAGTAGTTTATAGAAAAAGGAAGGGATGGTCTAGAACAGAC
CCCTGAGGCAGTTACAGCATTTCGATGAGGAGATGAGTGGAAGACATAGCAGAAAGGCCAGAAACCGTGTGCTGTGGAATCCAGGAACAGGGAG
CTTGTTACATGTCCACTCTGTTAGATGCTGCAAAGGTGTGGATGTAGTAAGAATACATTGGACATGGGTGAGCCAACATCCTTTGACATTGGTG
GCCTTGACTAAGCAGTTTCATTGTCACTTTGGAACCGGAAGCTGATTAGAGAGGATCACAGCGTGAATGGGAAGTGAGCAAGTGAGGGGCAGTA
CTTAGAAGAAATTCTCCTTTGACAGAGGAGTCTGGAAGTGGAGGTGTGGCACGGGAGAGAAGCCGGACCGAGCATAACGGTCTGCTGCTTTTGT
TTGTCTTAATTGTGGAGTGTTGTTTATATCCTGCTGACAGCAAACGGTAGAACGGAAATTAATGCTGACTCAGGAGAGATTCATTGCAGGAAAA
TTCTTGAGATGGTTCTGGTGAGACAGGAGGGAGAGAAACTGCCCTTTCATAGAGGAAGGATGCAGTCATGTGTCTATACATAGCTCCTCTGGTC
TTTTTACTTTTAAACACTTAGGTTTTTTCTTGTAATAAAAAATACTCATTTTTATAGACTTCTCAGGAAAGTTCACCACCTTTTAGCTCATCCC
CCAGTCTTCACTAGTCTCCTTTAGACCCCCTGACCTCTCTGAGCACAAGTTGCTTTTTTTCCCTGTACTCCATATTATGCCTTCTTCCAGACTG
AATTCATTTATAACCTTTGGGTCATGTAAATAAAAAATTTAACATCAATTTAAACTTTTGTGTCAACAGATGAAGCAAAAAATTTATTGCCATA
TCTTGGACCCAACAAAATGCGCGATTGTTTCTCCACTATAAATTTGGACCAGGAAGAAGTTTATCGCACCCAGGTTGTTGAAAAGTCTTTAAGG
TTGGTAGAAAAATTAGAAATTATTCATTTGATGTATATTCATGGTTTTATGTATTTTCCAGATTTTTATCAACAAATTGAGTTTCTTGATATTG
AAGCTAATATTTTTTATTTAAAGGTTTTGTATTCTATTATGAATTTATTTTTTAGATTTATAGAATCTAAGCATTTAAAGAGGCCTTGAATGTC
AGTAAGCTGCACTGACAACATTGATCTCCTTAGCAGTAGTTCTGGGAAGTGGTCATGGGGCATCTACTTCATAATGGTTCAGTCACCAAGAAGT
TCACACCTCCTAAGTAGGCTGCTTGGTCTTTGAACTCTTCTGACTCAGAAAGTGCTTATGTTTAATTGCTTAGTCATTATTAAAGTGACATCCA
GTCACGTGCCCAGCGCTTGCTCAGTTCTGGGTTCTGTTCACTCATGTCTCTAAGAGTGACCCCAGTGAAGTGCTTGCTCTCTCATGTGGCCTTT
CATTGCCTTAGGAAGTAGTTTTTCTTAGTTGTCCTAGCTCTGTCCCTTAGATTTCTGGGGAACAATTTTAGTCCATCTTTTATGTGACAGCATT
AAATGTTTGAAAACAACTATTATAAGCGTTCAATGTGCTCTCTAGGCTAAATAGTGCCTATTTGTTTGAGCTTTCCTCAAAGCACATCATTTTT
ACTTCTCCAGAGAACATATTAGCTCAAAGTTCACACAAGTTGAAGCTAGAGAGGTCGATGGCTTTCTTGGTAAAGTACTTGCTGCACAAATAGG
AGGTCAAGTGTTACATCCCCAGTACCTATGTAAGAAAGCCAGGTACAGTGGCTCCCATATAGTTCCCAGTGCTGGGGACATGGAGCGAGGAGGG
TCCCAGGAACCTCCTGGCCAGCCAGTGTAGACAAACCGTTTAACAACAGTCCTGTCTCAAAGAATAAGGTAGAAAATGATTGAGAAAGGTGATG
GATGTTAACCTCTGGGCTACACAAACACACCCTTTCTCCGCATCTGCATACATACAAGTTACTTACTAATGGATTATCAGGGAATAAGCTCTTG
GATTGTTTTTATCTGTCAAAGTGTAATTATTCTTTGAAAGTACTGCATACAGCAAATTCTTTCCCTCCTCTGCCTTGTAGCAGTGTTTGTGTTT
GTAATTTTGATATAGAACTGTGCTAAAACCAGGTGTCATGATACAGGCTTTAAATCCCAGCATTTGTGAGGCAGAGCTGGTCAGGTCTCTGCTG
AGGTTGAGGCCAGACTGGTTCACATAGCAAGTCCCAGGCCAGTCGGGCTACATAAGGAGAGTCTATCTTTAAAAAAAAAAAGAACCGAACCAAA
CCAAACCAAACCACCACCAAACTGCTGAAAGAAAGTCTTCAGAGACAAAGCATGGAGGATTCTGGGACTTCTTGGGACAAGCATGGTGAATGAA
ACAGTTGTCGAACTGTATTTTATCCCATTCGATTCTGGAGGATGCTATGGTACCTTGTTACCCTCTCCCACACACTTGCCTGTGTTTTCCTGTG
ACATTTGCCTGTGTCTCTTTCCTCTTCTTTCTTTGTCTTTTCTCCTCTCCCCTTCCTTCCCTTTTCCTTTCCTCTTCTCTATGCGGGGTCTAGC
CTTGTAGCCCAGGTTGGCATTGAACTCTTACTCCTACTTCAGCCTCACATATTGGTGGTGTACCCAGGCCCCTCTGCATAGCTCTTTGTCATTG
GGTTCTTACAATTTTGATCTCTTCATAAGAAAACTATATAGAACTAAAAGTAACCCTTGTAGTCGCTCCGTCAGTGGGTGAAGGAACCAGCTGT
CAAGCTTGAAACTTCAGCTCAGTACCCAGAGCCCACATGGTGGAAAGAGAGCACCAACTCCCACAGGTTGCTCTCTGACTTCACATGAGCGCCC
TCCTCAAAAAATGAGAATAGTACATAATTAAAAACAAAACAAACAACTAAAGGTCCCTTAGTAGCCCAAAAATATTGAGAGTAATGAATTTCAA
AGATTTTGTCACAAGCCTTATGCCCATATTTAGAATCTTAATTATTTTTCACACTTGTGCATTCAGAGTCACAGATGGCCTTTTTCTTTGTTTA
TGAATGATGAAATGTCACGGGAAGGCTGCATCATGTGTCATATAATGAAGGAAGGTAAAAGCACAGCTGGGATTTGAGACCTGAAAATATTTAA
ATTTCAGTTTGTTACAATTTTATATCACAAGGTAATCAGTGATTACAAAATTCAATAGTAATTTTGTAAATTGAGTCCATAAATTGAGTACTTA
TAGTTTGTAATATCTAGCCATATATGAGAAAATAACCCTTAACCTTTCTGAACGTTTGTATAGTGGAAAAATGATGAAACCAGTTTTCTTTATT
TCTTTCCACCTTTAAAAAGAAAATCATTTGAAAGATAATTTCAAGCTAGCTGTGTCACTGCCTTAGATCGTAAGGGTATAGCTAGCTAAACTAG
ATAATTCTTGCATTCTGAATGCCTCCAATCTTACTTTCTAGAATAGGGCAAAAATAATCCTTCTTCAACTTGATTTTATTATAACGTTCTTAAT
ACTTGTCTAAATTTTCATCAACCATTAAATTGGAAGTATCTACATTGTAGGTCTTTCTAACTTTTTCCACCTTTGGAATTTTTTCTTCAGTTTT
TATAACTCTGAATTATAAAACAGGAAATACTTTAGAATTGTAAAAAGATTTAAGAGATTTTGCTTCTAAATATCTCTAAATCAGAGCAAGTGCT
TAATAATTAACGATTTTAAATTTTAAGAAGATTCAGATATTTCATAAAGGAAGATATACAAGTATCTCCAAGACACATCGAAAGAGATCCAGTT
CCATGAGCCACCAGGAAAATACAGTTTAAAACCATTTTTGTCACTATTAGGACATAATCTGTAATGATAGAACTTTCCCCAGTTGACTGGCTGT
CTGAAGGGTCATTGCAGAGGGTACAGGGAAATTTCTGGGGGATAGGTGATAGGTTTTATGTTTAATTCTTTCTCTGAAAATTCCATACGTAAAT
GTTTTGTTTGTAACTCGTACCCACTCCGTTACCCTCCCTCATCTGATTCCCATGCACACTCTCTGAATTCCTTCTAGCAAGCTCTCCTGCTCTC
ATGTGTTTGTGGGTATGTATGGGACCCACTGAGTTTAATTAGGGCTGCTTGCATACGGGTAGGTGGAGCATGATGCACTGGGGCATGGGCAACA
AACAAATGGCTACACCACTGAGGAAAATGATTTCCCCTTTCCAGTAGCCACTAACTTCCCCAGCCATCATAGGTCAGTCAGTGAAGTCCTTGTC
GTGAAAGCATTGAGAACTTCAGTTTGGACAGTTAGCACTCATGTAGAGAGCCAGACATTAGTGGTGTAATCCCAGAGCTGAGGTGTGGAGCTAA
GAAGATCCTTGGGACTTCCTGGCCAGTCCATATAGCCACCTACATCAGTGCTTCAGCTACAAACCCTGTCGTTTGGTTTCGTTTGGTTTGGTTT
TTTAAAATAAGGTAAAGATAGATGAAGGAAGATGCTTAATATCCACTTCTGATCTCTATACTGATGCACACATGGGAGTATGCACACCCACACA
CAGGTCACACAGGTGCACACACAGGTGCACACAAGTCAAATCACTGAGACTGAAAAATTAGCTCAGTCTTGTTAGACAAGCATGAAGACCTGAG
TTTGGTTCCTCGGCACACATAAAACTGTATTGCAGTTGTTTGAAATGAGCTGGGCTTATATGAACAGGAAGGTCCCTGGAACTAGGTAGCCAGC
AGTGTGGCCAGACAGGGACTTTCAGATTCAGGGAAAGACCCTGTATCAAAAAATTAGGTGATGACACTCAACTAGGTGATTCACCTCTGGCCTT
TATACTCAAAACAGTCACACTTGTACACATATGTATACATATACAAAGAAATCATATCACTGGGCTAAGAATATAACTCAGTAAAAGAGCACTA
GTGTTATAAAAGTTAAAATGTATACAGTATTTACCTTAATCACAATGGGGGCAGATGTAAAAGAATTTTAATCCAGCATCATGCCTGTTCTGGC
AAGGGATCACATCCAAAGCCTTCTAGGACTCTGTGATCTCGCTGGAGTACAGGCTCACCCTTAGCACACAACTTTAATCCTAAACAACGAAGGT
AAAGTTAATTTGTAGAAGGAAGCAGCCGTGAGGGGCAGACAAAATGACAAGTCAGAGAAAGATTTGACAGAATGAGTCAGTGATGGCATATGCT
CAGCTCTCACAACATGAGAAAGAGAGGTGACTTAAGAGAGCAGCATAGAGGGGTGGGGAGAATTTTACCAACGTAGGTTGCAGATAAAAGACAG
AGAATAAGAAGCAGCCAGAAGATTAGAACAGATTGCCAGAGTTAGTTTGAGGCCAAGCAGAGCAATTCACTCAGATGCTAAGAGGAGCCAGTGT
GAATCAGTCAGCTTGGAGAGGAGTTTGGTTCAGAACAGCTGAGTTGAACCAGCCAGCCAGAGTTGAAAGAGCTGAACAGATGAGATTATTCAAT
AGTAACCCTCTGAAACAACAACTGTATCTGGCAAATAAAAGATACATTTACATACTTGCATAGCATACACAAGCTCCCTGGCTCAAACCTCTGG
AACCCACCCCCACTGCTCAGCTATCTAAGGTGCTAACAGTATTCTATATTTTGATTGAGGTAGAAGCTACACAGGTTTGTAATTTTATCAAAGA
CCACCAGCCTCTATAGATCAGATTTGATATTATATAATTCAAAATTAACTCCGGAAAGATAATAAAAATGCTTAATGGTCTATTTCTAATATAA
TTAAATATTTTATATAGCATTATTCATTTTAGATATTCAGTATTATACAGCTGTTGCCGGTTATATCTTTCTAAGATAATAATACGAATGGCTT
TGTCTTGACTCTCATTCATGTCAGCTTTGGGTGAGCATGGCTAGCCAGGTACTAACTCTTCTGTTCACAGGACCTTCTGCAAAGCTGTATGTTT
AGAATTCATTTGCCACCTAAAGTCTCCTCAGTTCACCTCGATTTCTATAGAATTTGTTTGAAATTCTCAAGAATTTTAGTTTATAATTTCATGG
ATGTTTGTTGAAAGTAAAATTTGAATCTGATTTTATTATTGGTACCATTTTAATAGTCATAATAAATATATATTATTTAATATAATGAGATTTC
TCTTTTCTCTCTAGTCCATATTTCAGTGAAGAATTTTATTTTGAGATTCCAAGAACTTTCCAGTATTTGTCTTTTTATGTTTATGATAAAAATG
TTTTACAAAGAGATCTTCGTATAGGTAGGTACTATTCATAATTATTTATAATTACAACATTAGTATTATGTTCATTCTTTCTGTAAGTCTTTGA
TATCTGCTTATTAAAGTAAAACCCATCAGCCATAGACGTTCACACCAACAGACTGGCAGAAGGTTGCTGTCTGTCCGTCCATCTCAGGCTCTCT
ATTGTGACATAGAGTTGCTGTCCATCTCAGCCTCTCTGAGTTACAGAGTCAGACTTCCCATCTAGAGAAAAAGAAAACCAAACAAATATTTTTA
TTAAAAAGCAAATACTAATTTTAAGAAAAACGATTTGTAGTTAAAAACAAACCTATAGGGCATAAGCAGATAATTTACTTCTGAAATGTTATAC
CCGGGGTCTGGGTTGTCCTTTGGGAAGATGAAGATGTCAGGTGTATTTCCTGTGATCTGAAACATATAGTAGGAAAGACTCGTTCCTGTGCGGC
TTTTAGGCCATGCTTGGTTAAATAGTCTTAAGTTGACGCAAGGTGGAAGTTGAGTCACTGCTTGTAACTGCACCGATGTCATTTTTAGAATGTG
GGCATGAATTGCTCAAAGGAATAAGCATCCACATAAATATTTTAAAACCCTCCCTTTTAAGGTTGAGGACTTGTCTTGTTTTCCCTTCATTTCT
CAGCTTAAAATTCAATTTAAAAGCCCTGAAGTACATCTTTCTAGTGTCACTTTCTACAATTCTAACTGTCACACAAAGATTTTTTATCTTTGTA
TTAGATTTCACTCAAAATATTTAAAAGAAAACAGATTCATAGGTTTAAAGATTTTTGTGTTTTGAATTTTGTGTGTTAATAGACTGATTGGATT
TTCAGTCTCTTGAGGACTATTGCTGCAGTTGCCATTCTACTACAAATCTGGTTCCGTACTTTAATGTTTGTTCTTTAAATTATACATCTGAAAA
GCCAGGCGTGGTGGCGCACGCCTTTAATCCCAGCCCTTGGGAGGCAGAGACAGGTGGATTTCTGAATTCGAGGCCAGCCTAGTCTACAGAGTGA
GTTCCAGGACAGCTAGGGCTACACAGAGAAACCCTGTCTCAGGTATTGAGTTTTAAGTTATAAAGAGCTAAACATAAAACTGTAATATGTTTTT
AAAAGTTTAAGTAACAACTTTATTAAGGTATAAGTTATATACCACAAAATTTTTTGTTTTAAAGTATATTATATTTAAAACTTATTTTTTAAAG
ACTTAGAGAAGGGATCATTGTACATTTACATATTATACACATACACATGCACATATCAGAGCAAAACATACGAAAAATGATAAGATTTTTGGAA
ATCCTGGGTAAAGAAAATAGTTTTATGTACTGTTCTTGCCAGCTGTTTTTTAAATTTGAAATTCTTTCTCCCCCCTCCCCTTTTTTTGAGACAG
GGTCTTTCTTTACAGCCCTGCCTGTCTTAGAATGCACCATGTAGACCACGCTGGCCTAGTGCTGGGATTATCAAGATAAAATATTTGTAAAATT
TGAAATCACACAGTATGACAATTGTATAATCACCACCTGCTTCCAGAAAGAAAAAAAATACACACACATACACGCACATATGTTGTTCTGTTTG
TTTTTCTATCTTCCTTTTCACTTGATAATTTAAAGTTGATACAGCTTCACATAAAAATTTATCATTTCTGTTTTCAACTGTTGAACTAAAAGTA
AGGTTATGTTGTAAGACATAACATACAGTTTGGGATACTTAAAAGGAAAGTATGTGTGGAGCAGAGACTGGGGGAACAACCATGCAGAGACTGC
CCCACCTGGGGACCCATCCCATAAACAACCACCAAACCCAGACACTATTGCAGATGCCAGCAAGAGCTTGCTGACAGGAGCCTGATATAGCTGC
CTCCTGAGAGGCTCTGCCGGTGCCTGGCAAATACAGAGGTGGATGCTCACAGCCATCCATTGGACAGAGCACAGGGTCTCCAATGAAGGAGCTA
GAGAAAGGACCCAAGGAGCTGAAGGGATTTGAAGCCCCATAGGAGGAACAACAATATGAACTAACCAGTACCCCCAGATCTCCCAGGGAATAAA
CCACCAACCAAAGAAAACACATGGTAGGACTCATAGGTCCCGCTGCATATGTAGCAGAGGATGGCCTAGTTGGTCATCAGTGGAAGGAGAGGCC
CTTGGTCCTGTGAAGGTTCGTTGCCCCAGTATAGGGGAATGCCAGGGCCAGGAAGCAGGAGTGGGGGGTTGGTGAGCTGGGGGAGGGGGGAAGG
ATGGGGTTTTTTGGAGGGGAAACCAGGAAAGGGGATAACATTTGAAATGTAAATAAAGAAAATATCTAATAAAAAAAATCAAAAAAAAAAAAAA
AGACATAACCTACCGTTGGGATACTTAAAAGCAAAGTATTTTTAAAACAAGGAAAGACGTGAATTACTCTTAAACTACCCTGTATCTCCTTTGT
TGGAGCTTTTGTGCGGTCTTTGAATGGTTAGTCACATGCAAAACGATGCTTCCCAGGGCTTGCATTGAGGAAGGAAGTGCTTTGCCCCAGCACA
CCAAGACCTTCATCTCAACATTATTTGCTTCTTCTCTCCTTTCATCCTCCTTTGTTCACCGTATTTCCTCTTTCCCTGAGACACAAGCGCTTTA
GGGAGCAGCTTTGATGGCTAAAGCAGTAGAGCTAGCTTAGTAATCTTTGATAAAATGATCTTTGAGGTACTTTGCACATTTGTTAGTAATAGGT
AAGATACTGTTTGTCCTTTCACTTAATTGGAGCATATAACTTCTTATTAAATGCAGGTGTTACTGCTCAGAATTAACAGCACACACACTTCATC
CACAGTGCTTCACAACCTGGAAAATTATATAGCATTGCCATTTACATTCTGTTTTTATCCTGAATATTAATCTGAGGAAGTGCTAACACACCAT
AGTTACATTAAGTCCAGCTGAGTCCTTCTACTCCCCTGATTCAGAATGACCCACAAAGGGCCTTCCAGTGGGCAAGCAGAAACACGGACATTTC
TTCTTTAAGGTATAAGAAAAAACAAACTTTATAGAAACAAATTTTTTAATTCTGTAATCTCAGCACTCAGAAGACTAAAGCAGGAGGATTACCA
TGAGTTTAGGTCTATCTTGGGCTAAATCGCAAGTACCAGGCTAGCCAGGGCTACATAGCAACATCCAGTTTCAAAAAAAGGAGTAACGGTGGAT
TTTTATATCAGCACATACAAATTAAAATATTTTCTGTAAAATATATAACAATGCTTATGAACCATAAGGTTATATAAACTATGGTTTCTCATCA
CTTTATTTATTTTCTGTAGGAAAAGTAGCCATCAAAAAAGAAGACTTGTGCAGTCATAGTGGTAAAGAAACTTGGTTTTCACTGCAGCCGATTG
ACTCAAATTCAGAAGTTCAGGTAAGTTAAATAATCAGGAGATTATAACATGAAAAACCAGTGAAAACCAACTAATTATACTAGTTTATACTGAT
AGAACTTCTAATACCTTATTTTACATTCTAATACCAAACATAGACCTTAAAGTAATAGAGAGCTGGAAGGAAATGGCTTTTCCATTTCGATTTT
TTTTTATTTTATGGATATATAAGTACAGTGTAACTGTCTTCAGACACACTTGAAGAGAGTGCCAAATCCCATTACAGATGGTTGTGAGCCACCA
TGTAGTTGCTGAGAATTGAACTCAGGACCTCTGGAAGAGCAGTCAGTGCTTTTAACTGCTGATCCATCTCTCCAGCCCTCGAGAACCAACTCTC
ATCTCTAACTTCCATTCATGTGCACGGCACCTATATACATACATATATATACATGTATCCATACAGGAATTAACCAGAAAATAATAAATTCTCA
TTTGAACGCATTTACTCCATTTCTAATAAACTAGATTTGTGGACAAAAAAATCTTCCTTATTTAAACCATCCCTGAATCTGCAATGCATAGCCT
TCATTCCTACCTCCCCTCCTGTTTACCTCCTTTTATTTGAAGGCGTATAATTATTCTTGTTAATCTATTCTTTTTCCCCTTGCCACATAAAAGG
CCTTGTATAAATTCTATATGTGAACAGAAATGTATTGTTGAAGTTTCTAAAAAGCTTAAGTCTCTATGACTTAAAAATTAATTCTAAGGGAATC
AATCTCTCTCTCTCTCTCTCTCTCTCTCTCTCTTATTTCTTTTCTTTTTTTGTAAACTTATTAACTATGGTAATGATTGTGTAGCCATTCACAA
GGGCATAACTCCAGGCAGGATTCTTTGTCTCTTAAGCAGGCTATAATTTGCTGCTAAGACTCAGAAGTTTAAAACAAAGAGACAAAAAGCATAC
CCACAGATACTTTGCAAATAAAATACGTCTCCATTTCTTCATAACCTTTTATGCAGTCTCTGTGATTAAATTCTAAAAAAAAAAAAAAAAAAAG
TATTGTGTGCAAAAATCCCTTCTTAAATACAGAATATAGCTAAGTACTTTTTCTGTTTTATCTACTCTACCCCGTTTTAATCTTCTGTCTGTCC
ATTTGACCATATGTATCATTCAGCTCTCAGGAAGAGCATTGTTGGTGCCAAGGGAGAAGCCATCTGAGGAACTAGAATGGGCTGATGGTGTTTT
CATATGTTCAGTTTAATGACCTTGAGCTTCATTTCTGTAAAAGGAGAATAAAATTCACCATGCTTGACTAATATGGATCGAGAATACTTTAAAA
CTTGATTTAGGGCTGGGTTGTAGCTCAGTTGGTAGAGTGTTTGGCTCGCATGTACAAACTCCTGAGTGCCATCCGCAGTACCACAGAAACTGAA
TGTTGTCCACGCACATTTGTCACCTCAGCACCTAGGAAGTGAAGGCCGCCAGCCTGCATAAACCCTGTCTTTAAAACAAACAAACAAACAAAAA
CCTGTTTGCTTCTATACAGATGTAAATTTATTACCACTACTCAGTCTGGTGGTGCCCTAGTTAGGGTTTCTGTTGCTGCAGTGAAACACCATGG
CCAAAACCAAGTTAAGGAAGAAAGGCTTTAGTCGGCTTACGCGTCAGTTTCCAAAAAAGTCAGGACAGCAACTCAAACAGGGCAGGAACCTGAA
GACAGAAGCCGATTGCAGAGGCCATGGAGGAGTGCTGCTTACTGACAGCCTGCTTTCTCACAGAACCCAGGTTCACAGACCCAGGGATGGCACA
CCCTACCCACAATCCGCTGGGCCTTCCCCCATCAATCACTAAGAATCTTACAGCCACACCTGGATCTTATGGAGGCATTTTCTTTCTTTCTTTT
TTTTAAGATTTATTTATTTTATGTATGTGAGTACACTCTACCTCTACAGATGGTTGTGACCCTTCATGTGGTTGTTGGGAATTGAATTTTAGGA
TCTCTGCTCACTCAGTCCCTGCTCGCTCCGGCCCAAAGATTTATTTATTATACATAAGTACACTGTAGCTGTCTTCAGACACACCAGAAGAGGG
CATCAGATCTCATTATAGATACTTGTGAGCCACCATGTGGTTGCTGGGATTTGAACTCACGACCTTCGGAAGAGCACTCAGTGCTCTTAACCAC
TGAGCCATCTTGCCAGCCCCCATGGAGGCATTTTCTCAGTTGAAGTTTCCTCTTTTCAAATAGTTCTAGCTTGAAGATAGTTGACTTAAAACTA
GCCAACACGGGTGGCTTTCTAATATAGCTTTACCAGATTATCCATTTTCAGTTTTTGACACATGACTTAATACATTTTCCATCTACGCCCACAT
GGTGTCAGTCATTTGAGTATGCTGCTCATGTGTACTTGAGGTTGGCTTCGATCTCACAGTGCTCTCTTAATATTGGCATTCTAGGCGTACACCA
CTGCACCCATCTAATCTGTCAGTGATTTGAACAAGAGGAAAGAGAAGAAGAGGCTTGTAAGATTTTGATCTGAAATGGAATTACTTAGAAGATT
CATTACAGTTTATTTATTTATTTATTTATTTAGTACGTTCTTAATGACCAGCATACATTTTTCTAGATTTAAGAATTTGATTCTGGGGGCTGGC
CAGATGGCTCAGCTGGTAAGAGCACTGAGTGCTTTTCCGAAGGTCCTGAGTTCAAATCCCAGCAACCACATGGTGGCTCACAACCACCCGTGAT
GAGATCTGACGCCCTCTTCTGGTATGTCTGAAGATAGCTACAGTGTATATATTAATAATAGTAAATCTTTGGGCTGGAGTGAGCAGGGACTGAG
TGAGTAGGGTTCAACGAGGACCTGACCAGAGCGAGTAGAGGTCCTAAAATTCAATTCCCAACAATCACATGAAGGCTTACAACCATCTGTACAG
TGACAGTATACTCACATACATAAAATAAATAAATCCTTTTAAAAAAAGATTTTGATTCTGAAAATATAGAGCTCTATGTGTTCTTTGTGTAAAT
CTTGGAGGTCATACCAGAAACTAATACTAGAGCACAGGCTTTATCTGACAGTGTATCTATAAAGCCATTATGTTTTATATATAGTATTGGCATT
TACATTTTTAGTACTGTTTCCTTCCGTGCCCATTAGTCATTAGCCTTTCCAGAACTTTTATCTTAAATGGTAATTACTAAGACTTACCCACTAC
TTAAAATAGATTTTAAAAGAAAAATGCTGTAGGAGGTGGGAGGGAGGGATAATACTTTTCAGGGTAATATGCTATCATTGGGAATATTATTATT
TAGCCACAAACAATCTGAAAATACCACACTAGCCTTGTGAGTATAAAAGATTTGAAAACAGCTTGAAACTAATATAGAGCCTACTCAGGTTTTG
TTGTTTACCCTGTATAAGTTTTATAATATCTTTTAAAGACATGCAAAAAGAAAAATAGATGGCAGCATAGAAAGCAATGAGATTACATTTAGCC
AGGACTCAATCTAATGATTACTTTTAGTAATTATAAAATATCTACCAAGTATAGCCCTGAGTACTTAATATACATTATCTCATTTAAGGCCCAT
AGTAGCACTATGAGGTAGGTATATTAACCTCATTTTGCAGATTATATTAATAGGATTAAACGACTTAGTCATAGAAGATGTGATCCCATTAGAG
ATCTGCCTAACTCCTAGTCACAGCACTGTGCTGTCTTTCAGTGACGTGTACTCTTAGTTATAGAAAGAATTCAACAGATTAGAATTGCTCAGGG
AGCATCAAAATATAAGAACAGAAGCCTACCCAAATTAGTGGGCAAAGGGGAATGCATTAGAAACTGTGTAGGGAGCTGTTTGAAGTCAAATTAC
AGGAACTTCCTCGGGAATGTGAGCTACATAGTTCCCAGACATTTCCACAGATACTTCGTGGTAATGGCAGTTAGGTTCTGACAGTAGCACACAA
GATAGAAATTCACATAAGTTAAAAATGATCCTTGAAGTGACTAAGTCTTCATAACATGGTTGACTGCCACAGAGAACATTAGTTTTTATCTGGG
GCTTTGATGAACAAACGCTGTGTAGTGTCATTCTTAGCAGTAGCCCCAACTCGGCAGCAATGTACTGTAAGGCAGGTGAGACCTGTGATCCACT
GGTTGATTTTTTTCATGTTTGTGGCTTTTCATTTTTCATTTTGAAAAAGGAATACTTATGTTTTCTTCTTTCACTCTCTTTTAAATAACTATAT
ACTTGCAAAATAAAGAGTGTAGCCGTGAATTAGTTAAACCGCTTTGGCTCCTAGCAGGTTGCGCTCTCACTGTACCCGCCCAAGAGCTCAGATT
CCCAAATAAAAGACAAACACACACATCCCAGTTAATTTTGATATCCTTTGACTAGCTGAATGGCTCGACACTTCTAAACCTCCCCATGGCTAGC
AAACACTCCCCTCTGATATTTCTGAGTTAACATCTTTAAAAATCTGTATTTCATCTCTGCTACCCCAGACCCAAGTGGGGAAGTGGCCCCAGAT
ACTTATTTGCCTGCAGGCCTATAAGTTTCATCTTTTTGCTTCCCCATCCTTGGGTCCCTTGTCCACACAAGTCTCGCCCCATCTCCCTGTCCAA
CCATTGGCTGCACAGCAATTCTTCATTATCAGTCAAAGCCAGTAGCGGGCAGGAATCCTCAGGTATCAAGAAAGCATGACTTTTGGAAGCCAAA
ATAAGACCAAACATTAGAACCAATTTCCAACAAAAGAGAAAAAAGATTCTGGGTGTGTGTGTGCAGGTGCCTGTCTATGTGTGAGCTTGTGTAT
TTACAGAAAGAATAACCTTGGGTCTCATTCTTAAAGATACTTCAACTCGGTTTTCAGACAGGGTCTCTCAGTGGCACCTAGAGATCACTATTGC
TAGGCTCGCTGGTCAGCAAGTCCCAGTGATCTACCTGTCTCTGCCTCCCTACGACTTCCAAGTACAACACTACAACCATCTTTGTTAAAAATTA
TTTCTTTTTTTTTTTGAGATTATACTATAGTTATATTATTTCCCCTTTCCTCTTTCCAAGCCCTCCCATATATCCCTCATTGTTGTTTTTCAAA
TTTATGACTTCTTTTTTCATTAAATGTTGTTAGGGATGTGTGTGTGTATGTCGGTGTGTGTGTCGGTATGGGTGTGTGTGTGTGTGTGTGCTCG
CGTGTGCGTGCGCGTGCGTGCGCATGTGTGCATATATTNNNNNNNNNNNNNNNNNNNNCGTGCGCATGTGTGCATATATTCTTAGCATGTACTA
CCACCTATTTAATCTGTATAAGGCTGGGCTGACTGTTTGGTGTCAGATGCCCAATTGGTGTAGCCTTCTGGGTAAGGGCTTCCCCCGCCTGGGC
TACCTCACCCCAGCCCACAAAGGCGTGTCTGTTCTCATCCTTGTTCCGCTTCTATTTAGGCAGTCATTTCTGTGAAACTTCATGCGTGTACCTT
CTGACATTACTGGGAGACATAATTCCATAGCAAACTCTCTGATCCTCTGGCTCTCACAGTCTCTCCACCCCTCACTCCCCTTTCACAATGATCC
TTAGGTGCAGGAGTTGTATTGTTGATGAATCTGTTGGGACTGGGTTCCACGGCTCTGCATTTTGTTGGTTGCGGTTTTCTGTAATGTTTCTAGT
CCTTTGTAAAGAGAAGTTCCCTTGATATGCAGGATGAGGATATAACGGCAGATATTCAGAATGTATTAGATACCCATCTTTATTTATACACCTG
TTTTCCTGGGTGGCTGGTGGGAGTTACTCATCTTCAGGCAGGCATGGCAGACGCTTTAGCAATTGAGCTATCTCCTCACTTAATGATTCCTAAA
GATGTCAACCATTCTTTTTATATGCCTAATGAGAGTACATATCTCTTCCTTTGTTTCTTTCTGTGAGTTTACTCTTAGTGCCTCTTGTAAAATG
CTATATTCATCTTTAAGTCTCTTTGTCCATATACCTTCCTCATCCTCAAAGATCTGACTCAATTTACAGAGAGAAGTTAGCTAAATTTTTGTCA
CTGTCCACTAAAGTCTAATGAGGTTGGAGACATTGCAGGGCAGTTCATTCTGGAGTCTGGGTTAGGCCCGTTTGTTTAGGGGATTGAGTGGATG
AAATGATCAGCATCTTTATCTCGGAGAGCAAGCAGTTTAGTTATCTGCCATTACCACTAATTAGTATTTGTAACTCAAATCCGTACCGATTTGT
TGGTGGAAGATCACTTATGGAATGAGGACCTCGGTGTAAAGGACCACGTGTCTGTGGAGATCCAGGAAGTGACACGTGTAAATGTCGGTCACTC
CTTGACTGAGGAGCTTCACTAGTAACTCCCATTTCATACCACGCACTTGATTGTTATCTTAGCCTTTTACTTTATCTCTTGCTTATTGGACTTA
CTGATTATTTTCTTCTTAGAAGCTCATGGTAGCAGGATTTAATGAGTGCATTTTCTACCTTTTTGGACAGAATTTTAATTAAAAAAATACTTTA
TGTGTATGTATGTTTTGCCTGCATGTATGTCTGCACCACATGTGTGCCTAGTGTCTGTGAAAGCCAGGTGTTGGACCCTTTTAAACTGGGGTTT
CAGACAGTTATGAAATGCCTTGGGGGTGTTGGAACCAAGGTCCTCTGCAAAAGCAAGCAGTGCTCTTAAGTGCTGAGCCATGTCTTCAGCTCCT
TGGCAGGATTTTTAGAATATTTATTGTCTGTGTCTGTGTCTGTGCCTGAGTGTATTAGGGGCAGCACGTGTGTGCAAAGCCCACAGAAGTCAAA
AACTGGCATCCGATCCCCTGGGGGGCAGGAGTCGGATGTGGTTGAGAGTCCACGCTGGGTTCTGGGAGTCAAATCTGAGTCTTCTACAAGAGCA
GTGCTTGCTGTCTGTCAACTGCTGAGCCATTTCTTCAGCCCCTTTTCTACCTTTTTAATAAAATACAAATCTTTTTCTTGACTCTGTTTTTTGC
CATTTTCCCCTCTGACATGAACCCTTTTCTTTTGAAACCATTGTAGTGTGCCTTCAGACTCGCTTCTTCGGACTTGATGCCCCTAAGCAAATGG
GCACGTCACTTTTATCAGGGCTCCACTGGCTGCCTGCTGCTCTGGTTTTGAAGCGTTCTCTTCCTTAGGCTCCAGTCCTGGTTATGATCCTGGC
TGTCAGTCTGTTCCAGTTGGTGTACATTTTCTTTATTCTCTTCCATACAAGTGATGATAATACAGCCATCCTCTTAGCTGTGAGATGCTGAAAC
TCACACCTTATAAGCATTGTTCCATTCTTTATTTCCTAGGTACCTCGATAAAAAACATCCTTTGCATAAGACTGTTGATTATGGGTGGGGTCTC
ACACAACATGTACAGGCAGCAAACCCACATGTTATGCCAGATGCAATGTTAGTTATGTCCTTTCGATGCAATTACTTACTAAGTCTTATCCAAC
AGGATAAGCTTGACCATCTGGATCATACTGTAAGTTATCGATCGGTATATACTTTTTCTTAATGGCCTTTGTTTTACTGTTTTGTTTTTTTCCG
AGACAGAGTTTCTCTGTGTAGTCCTGGCTGTCCTCGAACTCACTCTGTAGACCAGGCTCGCCTCGAACTCAGAAATCCGCCTGCCTCTCGAGTG
CTGGAATTAAAGACGTGCACCACCACTGTTTTAAATAGCCAGTTAAGAAGAAATCAAGACATTAAACACATGTTCAGTTAAGGGATCATAATAT
AAAAGCATAATTCATTATTATTATTATAGATTGATTTTAAAACTTTGTGAATTTGTATATGGGAACTAACAGAAATGAGCCATTCTATTTCTTT
TTAGATTTTTTTATTTTTTTGAAAAAAGGTCTCACTATATAGTCTTGATTGGCCTGGAACTCACTGTGCAGACAAATTCAGATCTGCTTACTTC
TACCTCCTCCTGAATGCTGGAACTAATGACATGTGTGGGCCACTGTGCCCCACTCTTTATCTCATTATTAAATTATTATTTAAGGAGAAAAAAA
TCTTGCCATTAATATAAGAGATATTATAAAGTAAAGTTTTGTCTTTCATCTGACAAGCAGTTGTGTCTAAAATTATTTAGAGGCAATTTCTATA
ACTAAAGCTGTCTTACCTGCTGTAGCGATGCATGATGCATGCCTGTAGCTCCAGCCACTCCAGAAACTGAACACCTTAGCCAGTGTAGCCAGAC
CTGAACTCAAAACAACCACTGAAACTCTGTCGGGAGCCATAACGTCGTGGTGGTGGTGGTGGTGGTGGTGGTGGTGCTGGTGTTAGTTGGTTTA
TTTGTTCCTCGCCTCATAAGTTTTTTAAACCAATTGAAACATACAGAGTTCTTTTTTATTTCTCTCTAAGGTCTGTGTGTTTGTATGAGTATCT
ACATGTGTGCATGTGCACCGTATGCCTATTTGGTACTCTTGGAATCCAGAAGAAGCATCGGGTGTCCGGGAGCTGGAGTTACAGACATTTATGA
TTGGCTGTGTGGTGCTGGGGTCCTCTGTGGGAGCAGCCAGTCTTCTTAACCACAGAGCAGTCTCATGCTGAAACAGTCTCAGTCATTTTTATGA
GGAAATTCTCAATATTTAAAATGTTCTATGATTTAAAGACTATTTAGATCTGAGAGGATTTTATCTTTATGCATTATTTTTTGCCTGTTTTTAC
AAACCTGTTTTCTGTGCATTTTTTGATACCAGGAGATAGCAGCTGCACTAAGGTTCCTTTTTATCTTTTCAGCTGTCTCACTAGTGCTTTGGTA
TGCTGTCTTCTTATTTATCCAACTGATATGTTTTTAGCAGATTTTGAATCTGTAACGTAATGTCTTTCACAGGTCTTAGAAAATTTAAAGCAAT
TCTCTCTGGATTCTCCCGTCTCTGACTTTCTGTACTGGGATTCCAGGCTCCCTGTACTAGAACCAGACTGTTGTGTATGTGTCTTATGTCTCCT
AAGCGCTCCCCTCCTCTGTACTTCATCATGAACATTTTCTATTCATCAGCCTTTCACTCCTGTTTATTCTCCTTTAGCTGTGGCTGATCTGATG
TTAGATATATCTCATTTCAGTTACTTTAGTACTTAGCTCTAAAGTTATCATTTAATTCATTTATAGTTTCCAGTTATTTGCTAGATTTTTCACT
TTATTTTCTGTTAGAATAAAAACAAGGCAAAATAAACAAACAAACAAACAAACAAATGGGTGGTATATATTTTAAGTTTGAGGCTGGCCTGGAC
TAAATAGCGAGACCCTATGTTAAAAGAAATTAAGATAACTATTTTAAACTCCCACTCCACTTACAGTCTTCTGTTCTTGTCTCTCTCTCTCTCA
CACACACACACACACACACACACACACGTAACACTAAAAATTACTACATAGTATAGACCTTCAAGGTCAGTATCAAAACGCAGAGGTTGTAAAT
GCTCAATGCAGGAATAGTGAAGGACCGAGTTTGTAAGATATATATGATTTTATCAGTATTTATACCTAAATTTTTTTTTCTGGACTATTTTTTT
AGTTCCATATTTATACCTAGATTTTTTTTCTGGACTATTTTTTTGGTTCCATATTTATACCTAGATTATTATTTCTGGAATATTTGTTTCTGTT
TAGTTCCAACTCTACATGACTGCTCGTTTTCTCATTGACGCCTTTTGTTGTTTGCGTTAGAGCGTATCTCTCTCATTCACACCTTTGCACTATG
ACTCATAACTGTATATTGCTCATTGTGTAGTAGTCATCTAGCCTAAAGTTCACAGCTAATGGTCCTGACATTCAGTATCATAAGAAAAGTCGGC
ATAAAGTAATAAAGGTGATTCAGATTATATCTTTAAATGAATGTCTAGAAGGCCTCTGAAGAGAAGCTGTAACTAGAGTGCAAATATCCCTACC
CCTGATACTATCACTCAGTACACCAGGCCCTAGACATCACACCCCAAGATCTCCACTTGGCACTCGGAATTTGTCTTGTCAGCTATGTGGAGGT
AAGGCAGCACTAATTGTTAACCGGTTTAGGATTGCTCATCGCCAGTAAGCAGAAGAGATACCCAAGCTCTGGATTACATTGTCCCTAACCTCTT
TCTTGAGGAAGCTAATGCCTTCAGTTCCTTAAAACGTACTAAATTGCGCTTTCAATTCATCTCTTACCAGCTGTGTGTAAGAGAGAACTCTATT
TTATTCCAATTCTTCCATCAGTGCTTTGACCGTATAGTGTCACTCTTACAGTCTCTCTGGCTAGGTCATGAGCTTCTGGTCAGTAGTGACGGTG
TCTTACACAGCTGTTTGCTCCTGTGTATTCCCATAGGACTTCCTAAAACATAGCAGATTCTAAATCAATCTTCAGTAGTTGACAAGGTATATTT
TAAAAAACTGATTCTTACTATTTTTAACTTAATTTCATAGCTTTAGAAATTAATTTATTTTATGTTTTCAAAAAATATAAGTGCCTTCTTTAAG
GAAATACTGTGGTCATTCCAGTATATAAACATTTATTAAGCAGGTATAAAGTTCCTAAATTTCTACCTGTCATATTTCATATAAATATTAGAAT
GTTTTCTCTCACAACTTTGCTATATATGCGCATGCATGCACATACATTGCTGATTATTAGCCATTTAACAAATAAATCTATGCAGAGATATGTA
ATAGTGGTTAGTGAATAATTATGTGTGCCATTTGGATAGAAGTACATTGTAATATTTTGTTAGCAAAATAAAATAGACTGAGTTTGATATTTTC
TTTTTGTCCTAGGGAAAGGTTCACCTTGAGTTAACACTGAATGAGCTGATAACAGAGAATGCAACCGTGTGCCAGCAGCTTGTTGTACAGTAAG
TATATCCTTTTTATTCATTTATTTCTTTTTGTCAAAACCACCTAGAAATAATTCTCTTTTATACTCTTAGACTAATTTCTTATTTTAAATATTT
TAGAAACTTCAAAATAAACCTCTCATGCATCATTTCTTTACCAAACTGAGAATCCAGCTTATTTTTTCATTTAATCCACTTAAGCTATGGCAAG
TCATTAATTACTCAACAAAGTTTTAATGAATGTGGGTTGAGCAACAAATGTATGTAAGCATTAAGGCATGAAGGGTCATAGGAAGGAGCCTCTG
GCTTCCTAGCTCCGGAGACTCAGAAAGTGTTTCCAGGGGATTCTTCTCCGAGCACTCAGTCACTACGCAGAGCTTCAGGGAGCCAGGGATCAGG
GTGAGGAGGTCATACGACGTGATGCTCAAACTACGTGACCACCTAAGATTGCTAGCACCTTTCCCTCTAGGAAGCTCTCAGTAGTCAGTCCAAC
TCTGTTGTGAGGTCATGTTCTAGCCTACTCTTCTACTTCTTGCCTCTGTAAACGCTTGCTACCAAGCGTTTCTCATTCTGGAACTTTCTGTGTC
TTTGCCTCAGTTGATATCTGAATGTCCCTGAGGTTATGAGGTTCATATCTACATACCTTAGGAACGAGTTAGAGCAGTTGTTACTTTCCTATTG
CTGCTCCACACTATAATTCACTCTCCTAATGATGAAAACCCCAATTCCTTGAAGCATTGCCTTTCTGTCTTGTGTGTACTATATGATCTGAGGT
GTTCAACTTCTTTCTGCTTTTATTAGCTATCTCAACTAATAAATAATAAAGAGACTAAGCGATTTTTGCTGTGTGTGGATGACTTACCAAAACA
GTATGCTTAACGCATTTTTTCCTAAAAGCTTTATAATCATGGTAACATTTAATAGATTAATAAAGTAATAAAAATTTTTTTATTTACTAACAGT
TTTTGTCTTACTGATTTTCAAATTCCATTTGCCTATTGAAAATAGTCCAAAGCCAGGCATTGTGCTATCTGCCTTTAATCCCAGCACTCAGGAA
GCAGAGGAGGTGGCTCTCTGTGACGTTGAGGCAACCAGGGCTACATAGAGAGTTCTAGGACAGCCAGGGCTACATAGAAAGTCCTAGGACAGCC
AGCCAGGGCTACAGAGAGGGTTCTGGGACAGCCAGTGCTACAGAGAGAGTTCTAGGACAGCCAGGGCTACACAGAGACCCTGTCTTGACAACAA
GAAAAAGACAGTAGAAATGAGTGTGCAGACTTCGCCCGCAGACTGAGAGCCTGTAGTGGAATTCTTTGGATAAGGCCCTCCTGCTCCTCCCTTC
CCTGCTTGGGTTTAGTGACAGTAGTCAGTCATTTGAAGTACTAGTAGTTGGATTACCTTATTTGCATACTTGTTTAGGTTTTTCTCTTGGGTGC
TTGGCTTAGTTTTTATTTATGTATATTGTGTGCTTGTGTGCGTGTATGCCATACATGTATAAGTCCTTACAGAGGCCAGAGATGTTGGATCTCC
TGAAGTTGGAGTCATAGGCTGTTGGGAGCTACCTGATTTGAGTACTTGGAACTTACCTGGGGTCCTCTGGAAGGGTATCCAGGGCCCTTACCTG
CTGAGCATTTCTCCAGCTTCCTGTTGTTATATTTTAGGATGTAGAATATTAGCATGATTTCAAAGGAAAAATTGTATCAGAAGGCATTATTCAT
CGGTCTGGTTCAGTTCCCTCTTCCTCACACTCAGCCCTAGGCAACCACAGTCTGATTTCTGTGTCTGTTTTTACTTTTTGGTTAGACTGGTTTG
ATATATTTAGTCTTTTGTGTCTGGCTTCTTTTACTTAGTATTCTCCCTTTCATACTCATTTACATCGTTGCCTATAGTAGTAGAGAATCTTCTT
TCCAGTGTTTCCAGTGTCTCCTGAGCTAGGCACACGGGTTCTACCACCCAGACAAACCCCAGCCCCTTATTTTTTCCCATGACTTGACAGATAA
CTCAACAATGTACATGCTACTTAAATAAGCCCAATTCCATACTCTTCAGTTTCAGGGCTAGATTTCAGTTTTTCTATTTTAATGTTTTAACTTG
AGGTTATGGATTTACAAAGTACAAACAGGTCTTTGTGCTCTTTACTTAGTTTCCCCCAGTATTTACATGTTACATGACTAAAGGAAATTTTTAT
TCCTACACTTTGTTGTGTATATACCACTTGGTTACATGTATGAATGTATGCAACCACCACTACAATCAACGTACAGTACATACTGTCTTCTGAA
GGCTTTTATTCATACTACAGCTGATAGTCATGCTCCTCTCTGCCACTCTCCCGACTCTTTTCAAACCTCTTTTCTGTCTCCAAGTCTATAACAT
TGTCATAACATGGTGTGAGAGCTCCAACTGTGACTCACTGCTGGAACACGGAACTCAGTGCTTCCTTACACGCTGTGAGGTCCTGCGTTCAGTC
TTGAGTAAACACACACAAACATAGGCACACACAACTAGGAGAAATAGAACCTAATTGTGTATAGCCTTTGAGATTGGGTTTTGTGCAAATCCAT
ATGAATTATGTGTACTCTCTTGTAGATAGACCCTATCATTGTAAAAAGTCGTTCTGTGGGAAGGCGTTGCTAGAGAGATAGCTCCATAGTTGAA
GAGCACTTGCTGCATGTGCAGAGGACCTGGACTACTTCCCCAGCTCACAACCGTCTTTAATTCTAACTAGGGGAACCTAAGAACATGGTCTGCT
CTCTGTAGGCACCAAGCAGGCATGTGGTAAACAGGCATACATGCAGACAAAACATTCTGGGGCTAGAGAGATCACTTTGTGGTTAAAAATATAT
ACTTCTCTTATAGAGGTTCCAAGTTCAGGTCCCAGTGCCTGTAACCGAATGGCTGACAACTGCCTGAAATGCCGTAACTCCCATGCATTAAACA
CTTATATACTCGTGGATACATGACATCCTTCCAGTATACATCAATAAATAATAGATAAAAACTATTTGTAAAGAGCCATGTTCTGTTCTGTGAA
TTTTTTCCATTTATTTTTAATCTAATTATTGATACACTTAACTCTGTCTTGTTTCTGTATACTGTCTTCTATTTTCTGAACTTACCTGAACATA
AGTTAAAATTATGTTTGGTTTTTCTTGGATATTTTCTTTACATTTCAACTGTTTTCCCCTTTCCAGGTCTCCCCTTCGGAAACCCCCTATGTCA
TTTCCCCTCCACCTGCCTCTATGAGGGTGTTCCCCCACCCACCCACTCCCATTCTCCCACCCTGGCATTCCCCTACACTGGGGTATCGAGCACC
CTCAGCACTGAGGGCCTCTTCTCCCACTGATGTCCAACAAGGCCATCCTCTGCCACCATGAGCCAGCACCATGGGTCGTTCCATGTGTATTCTT
TGGTTGGTGGTCCAATTTCCGCGACCTCCGGCAGTGGGGGCGTCTGCCCTGTTGACACTGTTGCTCCCTCTATGAGGCTGCAAACCCCCTCAGC
TCCTTCAGTCCCTTCTTCAACTCCTCCATCAGAGACCCCCAAGCTCAGTCCAATGCTTGACTCCAAACTTCCTCCTCTGTATTTGTCAAACTCT
GGCAAAGCCTCTCAGGAGGCAGCCATATCAGGCTTCCATCAGCAAGCACTTCCTGACATCCACAATAATGTCCAGGTTTGGTGGCAGTATATGG
GATGGTTCCTGGGTGGGACAGTCTCTGGATGGCCTTTTCTTCAGTCCCTGCTCCACACTTTCTCTCCATATTTCCTGTTCTATTTATAGTGTCT
TATTACATAGGTCCTTTAAAGTGGTTGTCTTGGGTATCAGACTATGTGTACATAACATATAACCTCCTGTTGTCAACAGTTCACCAGTTACAGA
TATCTTGCCTCCTCTTTTACCTTTTCCTTGCTCCCATGTTTAATATATTTTTGTATTAAACATTGAGAATTGTGCTAGTGTTAGAATTCTTTGC
ATCAGGCATTTAACTTAATCCAGAAAAAAGGAAAGCATAGTATTATTTACACATTTTTCTCTGTCCCTGTTCCTTTCTTTCTTCCTGAATTAAC
ATTCCTTTTGTCATTCTCTTTAAATATCCTCCCCGCCCCAATTTCCCATACTTCTAGGGTAGATCTAGGGTGACAAATACTCCTCATTTTCCTT
CTCAGAATCTTTTGATTTTTCTCTATACACACACACAAGCTACAACCAGTTCCTCTCTCACCAGCTAAGCTTTGGCCGTGGGTGTGGGTGGTGA
TAGCATTAGTTGTTTTTCATTTGGTTGGTTGGTTTTTGGTTTGTGTGGGTTTTTTTTCTTTGTTTGTTTTTTGTTCTTCTGCTTTTTGCTTTTT
GGTTGAGGAGCAAGTGGACATTAGCTTATGAAGGTTTGTTCTGTTGGGTGCCCTGTTTCTTGCTCTCCTTTGGAGGCCGATTTCTGAAAGACAT
TTGTTTAAAGATACAGGAGTTCGGGAGTAGTGTGTGCACAGGATTATTCACAAATGTGTACAGCATCAAAGCTGACCAATAGATACTTACATGA
CAGAAAATTTACATATTCTCTCATACTTTAGTACCGACTGCCCCTCTGTGGCTGTTCAAGACATGAAATAAGCCATATGTGACTGATATTTTGT
TAAATTTTAATGTAATAGCCAAATATGGTTAGTGGCTGCAATATTAGTATAGCTCCAATAGAAAGCAGAAAGCCTGGAATCCTTGTTAGCAATG
GCTGATCTAGGGCTGGCTTAAGGAAATATATGATGAAGCTAGAATACCTTATGAAATTAGAATGTAAGGAAATACTCCAAAAAATAATGGAGAG
ATAAATGAATGAGAGACATTGAAATCAATCTGAATAAGTTTCAGAAGGCCAATTGTGGGACAGCTTCAGATTTGAGGACAGCCTCAAAATTAGT
ATTAATAATGGACTAAAGCTTGAACAATAAATATTCATGAATCACTACTGATACAATACACACAATCGTGTAGATACATAGGAAGAAACAGTTA
CTTTTCCTTACAGTATAATTCTAATTAATGAATTTAGGAACGGTGATGGAGATTGAATCAGCACTGGCAAATACCACAGCTGGGTTCAAGCAGA
GAGACAAGCACTCTATGCTCTCAGTTATATATGGATGCACAGAAAGCCTTTATCAAGGAAGCCGGTACAGAGTAGTTGTTCCCAGAGCCTCGGG
AAGATCTACGGGAAGGATTGGCAGAAGGAGGATTGTTTAATGGGTACAGAGCTATAGCGTAGGAGGAATAATTTCTAATAATTTATAGCACAGA
ATAATTATGAGTGACAACTGGATATTTCAGCACAGCTAGTAGAATTTTGAACATTCCCCAAAGAAATTATGTTTAGATGTTAGATATGCTAATT
TCTCTAATCTGATCATTACTGTGTGTGTGTGTGTCTGTATGTATACATATATCTGTGTGTGTGTGTGTGTGTATTGGTCTGTGACACTGTATTC
ATAGGTATGTACAATTACATGTCAGAAATAAAAGCATATATGGGGGCTGGAGAGATGGCTCAATTGTTAAGGACATTGACTCTGCTCTTCCAGA
AGACCTGGGTCCGAATCCTAGCACCTACATGGCAGTTCACAACTACCTGTGACTTCAACATCTGATGTTCTCACATAGACATACATGTAGGCAA
AACATTAATGCACATAAAACAAAAACGTAAATAATTGAAAAATAAAAGCATATAATTAAAATATAACTGAGATCTGGAACCATGGCACAGTGGT
TAAGATCACTTGATACTCTTTCAAAGGACCTAGGTTCTGTTCTAGCACCTAAATGCAAGGTGGCTCATACCTTCATTTCCAAGGCGATCTGATA
CCCTCTTCTGATCTTCACTGGTATCAGACATGTAGGTGGTGTACATACATGTATGTAAACATTCATAGAAATAAATAGCTAAAGTATAATAAAT
AGGATGAGAAGTATCTAAGAGTACTAAGTATGTTAGCCTAAGTATGATTGCATTTTTGTATAGTCTCAAACAATCTCCCTCTGAACTTATTTCA
TAGATAACTCTTCACAGTAGCATATCAGGTAGTCTTGGTACTATGTACACAAAAGAACAGCGAGCTATCTAATTGAACACTCCCCCTCCAATAA
TAGACATGGCCCCCAGTGGAGGGATGGGGCCACCCATCTTCAAAATTTTTGACCCAGAATTGTTCCTGTCTAAAGGAAATTTAGGGACAAAAAT
GGAACAGAAACTCAAGGAAAGGTCATCCATTGTCCATCCCAATTTGGCATCCTTCCCATGTGCACACACCAAACACTGACACTATTGCTGATGT
TGTGCTTGTAGACAGGAGCCTAGCACAGCTGTCCTCTGAGAAGCTCTATCAGCAGCTGACTGAGACAGATGCACATACTTACAAGCCAACACTG
GACTGTCGGAGACACCTTTGGAAGAGTTACGGAAAGGATTGAAGCGGCCTAAGGGGATGGCAACCCCATAAGGAAGACCAACAGTGTCAACTAA
CTGGGACCCCTGCAACTCCCAGAGATTAAGCCACAAACCAAAGAGTGTACATACGCTGGTCCGTGCCCCCTCCCCCAATCCACACAGAGGACTA
CCTTGTCTGGCTTCAGTGGGAAAGCCTACTCCTCTAGAGACTTGGTGGCCCTTTATCTATTTTTAAATATATAAAACTATATTTAAGCTGGGAC
ATTCACCCCAAATAAAACCTCTTTGGGAGTAGAAACTAGACATGTGTCTGATTTATTTAAATCGTATTTATTTTGATAAAAATGGGGGGGCATC
TGATTCCATTCTGGTCAAGAGCAAATGTCAGACGAACTTAGATTAAGTAATGTGAAAGCAGTTATCACAAGGTCAGAGACTGGGAGGACATGGC
AGCTGAATGCAGTGTAGAATCCCATACTGCATCCTGGAGTTCAGCAAAATTTGAGTGGCATCTGTAGATTAATTAGCGTTAGTAGCATTAAATG
TCAGTTTCTTGATTTCAGTAGCTTCACCTTGGTTACACAGAATGTTAACATTCAATGTTAGGGTTTATAGAAACTCTGTATGTTGTTCTTACAA
CTGTTTCAAAGTCTGCAATTATAACTAAGTGTGTTTGTTTTGAGTCCTGGTCTCCTATAGCCACTGTACTGTCACATAGCACTGCCACTGTCAC
ACACCTCAACTTTTACACATTCAGTTTCAGGTTTGTTCAACTCCTTTCCAGAGTCTTAGCAGATATTTGAGTCTACCTGTGTTTCTTTAATCAG
ACCTTACATTTTTTCCATTTCTAGACCTTTGTTTTATTATGCAAAAATGTTTATTTTGAAATTTTTTAAATTTAGAAATACAAAACCTCATAGG
AAGGAGGAAAGAAACAAAAACCACAAAGCATCCAGGCAGTGTTTTACTTCATGAACATGTCACCCCCTCTGGCTCACTCGTCCCATGGTGAGAA
AGGAAGGACTCAGCCACCCACAGCAATGTTATTAATCGGGATATCCATCAGTTTCACCAACAAGCCAGTGAACCCCATGATAGCAAATCCCATC
GCTGTGGCCATGGCAGTCTTCTGGAAAATTCTTTTCTATCAAGTTTGGTACATCTTTTAGTCAGCCGCATTGAGTCTTTTACAAACTCCTGACT
GGGCTCTACAGACTGCATTATCTGATCCATGTGTGTGGGATCACGGTTTGAGAGGGTAGAGGCACGTAGCCTAGGAGAGAATTCAGCCCTAGAC
CTTTATTTCTTAAGGCTTTCTGTGTCTATGGTGTGTGAGGTAAAAATTTTCAGTGTTCTTGTCTACAATTACTTTTTGTCGTTTCTCCTTCTCT
TTTGGTGGTTTTTCTTCTCACTGTAGGCCATGCTTATTCATTGTTTGAGTTGGACATAGAAGTCACTAGCTGCTCTGTGTTGCTGTAATGTACT
TGAATTGGTTTTTAGGTATCAGTTAACTTACTTGGAAACAATTCATTCTGTTTATAATTTGGTTTTAAGCTTTGTCACACAGATGAGAACAGCA
TATTATCATAGAGCTTGCAAGAGGCCCACCCAGCCACAGAAGAGAACTGGTAGTTTAAGAATAATGCCATTAGTCGGAAAATATGAACCCCAAA
GTATTTCTTTGGGCTGTACTTTTTTTGGTTAGTTTCAGAAATTTGTGAATTTTATTTTTGATGCTCAGTTTAGCAGAAATGAGTCTTGTTTTCA
ATTTTCTCTTTACTTGGCAATACATCATTTGCACTTCACCCATGAGTGAGTCTTGGAGAGAAATAGAGGATGTTTCTCAGTAGGGGTTTGTGAA
ACAAAAACAAAACACTTGGCATTTATGAGCCTCTGTGAGGGAGCAGGAAATGAGACTCAGACTTTAAACCAACTAACTTAGTAATAATTACTGA
GGAATAAACCAACTAACTTAGTAATAATTACTGAGGAACTGTGAGAATCGCATCATTAGAGCGTGTTCTTTGCTTGCTATAAATACAACCATAG
TCCGTACAGTAGATTGGATGGCCTTGGCTTTGTAGTGCAGTTCATTTTGTACAGAGTGATATAGGAGCTGCTGTTGAGACTCACCATGAGAGTC
TGGAGAACTATGTGAGGCTTGCCCTATATTTATGAATCAGATTTTAAGTAAAACCACAACCATTTCCTTGTGTGTCATTCATGCTTTAGCAGAG
GATATGGTGCCACACAGCTGGCCTGGTTTGTAGTTTCTCATGTAACCCTACACCACTTTCTTTTCCGTTTTACAAGCCACAGCAGTTTTGATGT
ATTATCAAACCACAAAATGTACCTTGAGCATTTCCCAGGAAGAACTGTGCTATGCTGGTCTGAGTACCCATTGTCCTTGCTGGGTGGTTCTTTT
TGGGAAGTACTCATGTAAATAAGTAATTTGCAGTAGTTGATACATTGGAACTACAAAAATGCAAACAGTGTTACGGTCTTTGAATATATAGATT
TCTATTGAAAGATAGTGTTTTAGGTTTGGTAGTGCAGCTCAGTTGCAGGGCACTTGCCCTGTGTATATGACTGAAGCCAGGGACTGTTCCCAGT
CCACAAGAAGGAAACGACAGGGAGAGAGAGAGGGGGTAGATACTATGTTATGATGTTAGTGCTAACATATTAAGATCTGTTTTGTTCTACCTTA
GCATCAAGGCCTGCCATGGGTTGCCTCTCATAAACGGACAAAGCTGTGATCCTTATGCAACAGTGTCCCTCGTGGGCCCCTCTAGGTAATGCTT
ACTGAATTGTTACTGCATTTTCAAGTTTAAACTTCGATTTAAGAATTGGAAATGTACAGTTCTTAGATCAAGTGCTGAGTAACTCACATAAAAT
AATGCTTTAAAACAATTTTTTGTAAGTTCTAGAGCACACTTGGTAAACAACTGAGTTCTGTTATCACAGAAAGATACTGGGTGTTATTTCAAAT
CTGCTATGTGCTTATTAGGCTCTGGTGAAAGCTTCAATCTTATGCCATTGTAAAATGTACTTTAAAGTATATTCCTGGCCCAGGTATGGGGGAT
GCATGCCTTTAATTGAAGTATTTGGGAAGCAGAGGCAGGTCGATCTCTGTGAATCAAGGCCACCTTAGACTGTATAGCAAACTACAGGCCATCC
AGGATCTAAAACAAAACAAAAAACCAAAAAAAAAAAACAAATCATATACACCTGGCTGCCTTTACTCATTAACATTCTTACTTTCAAATATCCT
CAATTTTCTGTAAGTAAAAAAATCACATTTTAGTGATTGTCAACTCAAAATAGCAAAACAGTTTCCATACCAGTATCTGGGAAGCCGCTTAGGA
CTGAGAACTGCCTTTTCTGTTCATGTGTCGGTGCTTTGTGAAGAGACCTCCAAACTCTAGAAGAACTACTGAGGTCCCAGGCCATGGTGAAGTG
CAGCATGATGTTGAGTAATTGGTCTATAATTTATGAAAAACCAGTAACACTTTGTGGTATCTATATTAGATAACTTCATTGTAGTTTCTTTACA
AGACTGACTAGTCTCGAGTTTGTGTGCAACTTAAAGCTCTTTGAATCAACAAACAAAAGCACAAAATTTGTTATCACCATGACTCCCCTTTATC
ACCTAAAGCTGTAGTCTTTGTTTTTTCGGTCATCAAGTCTTAAAAAAAGTGTGTGTGTGTGTGTATCAAAGCACCTGATACCATGGAGCTTAAA
TACTGATATGCTATTATTACAGATTGGTTTTAAATTTAAAAGCAAAAATGTATATGGCATTAAATGCTGCCAAAAGACTTTTCACATCAATGTT
AAAAATGTTCATCCTCACTGGATATTGGAAGCATAATGATAACCCCTGTACCTGGGCAGCAGACGTAGAGGCTCACACATTCTGGCCAGCCTGC
ACTACTCACAACCATCTCTCACAGACAAATGACAAGCGAAACCAGCCAGCCTAGAAGAGCTGCTTGTTTCCAGTAAACCTCTTTTGTAGTTCTG
TCATTGAACCAGACTATTGTTACTAAGCAGTTACTCTACCAGAGAGAGATTCTCAACTCAGGAAGTTGTTCTTGGTTTCTGTTTAATGATTTCT
ATCTCATGACAAATTCTTTAGTGGAGCTTTTAAAGTAACATTTCACAAGAACTATTTTTTTAAGATTTATTTATGTTTATTTTATAAACATTGG
TGTTCTTCCTGTGTGTGTGTGTGTGTGTGTGTGTGTGTGTGTGTGTGAGAGAGAGAGAGAGAGAGAGAAAGAGAGAGAGAGAGAGAGAGAGAGA
CAGTGCATGCCAGATCTCCTGGATGCTGGGAATTGAACCAAGATCCTTTAGAAGAGCAGCCACTGCTCTTAACCACTGAGCCATCTCTCCAACC
CGAAACTTTTTTTTTAATCACAGTTCATTTTGCTATTATACTACCGTTGCACAGACATTCCCATTGGTTGAGTCTAGTCAAACAGACAAGACTC
CCAGTACATTTCTTTATAACTGCCTCTGAAATTGTCCATTCACTGAGAATTAGCAGGCTTTGTTTTTTTTTAAGTTTCTGTCCTGGCTCCGGCC
CTCAGAAGTACACTGGGTTCAGAGTTACCTTTCATGCTTCTCAGGTGTTCCAGCATCAGTAAAGGCTTGGAGGCTTGGGCCTTTCTTCTCACCA
CTCCTTCTGGTAGTGTAATCGGATCAGCTTCTCCTAGCCGTGGAGACAGTAACCCAATCTTGCCATGTATAGATATGTAAAACTTGCATCCTGT
GTGTGGATATTCATCAGATTCAAGTTTGTGAGGTTTATATCCTTGGCTTTTTTCCTTTGCATAATATGTAAGTTACTGTCTTCTCTCAGTTGTC
CTAAAATGTACGCAGAGGTTAGGCATCCTAGCTGGGAATAGCTTGTTTTATAATTGTGTACTATTAAGTTCTACAAGGTATAACTCTACTACAT
TGGAACACGAATGACCAAAAGAAGACAAAAGTAAAGAAGAAAACAAGCAATCCACAGTTCAATGAAGTCTTCTATTTTGAGGTAATTTCTTGTT
TTATATAAATGTTAATGTTTGATATGTAATTCTAGTTGATTTAATTTCGTAAACCATAGCCATCAAAATTGTTTTATTAATTAATGCAGCTAAT
AATCTGTACCCAGAGATGAGTAGAGCCATAATGTCACACATGCAGCTCTGGTTGGTTTGGAAGTGGGGAGGGCTAGAGCCCCTACCACATAGTC
ACATTGTCACAGCTCCCTTGAGAGCCTCCCCACACTTTCCAGTCTACCTGACACATAGTGACTTCTGAGCAGACTGTTGGAAATCCTATAGAAA
TTTAAATATTACCATTCAGAAGATCTGAAACCTTTTCCCAGCTGATATTAATGATCAATTAAATGTAAACCCTGCCTTCCATATCCTGTAAAAG
GGTAATTTTATAAGGAAGAACGGTAGCAAATCTGCCAAATATGAGACTACTTTGAGAGTTTGAAACAGTTTACGATTATGCCTTTTTACCATTA
AGTTCTCTAAATCTAATTTACTCTGTAGATGTAGCTTGTTCATGCGGATCTCCAGCTGCCTTGAGTGAATCCTAACCACTGGACCACCAAGCTT
GCATCTTCAGCTGCTTTGAAATTGCCCCAAATAAAGTGCTTTGGCATTCTTTAAAGTATTTAGTCTTTTTTCATATTAATTGAAGACTATAGAA
AAACTAAAAATCAAAATATCTAAAAGGTTGTAATAACAGAACAAGTTGCAATTGATATTAAGATGTGAGAACAGGTTGTAAAAGTGGGTAATTA
CATCTTGAAGTTTTTATTTTGTTTTTTTCCTCTGTTTTGGAGACGGCACCAAAGTACCCCAACTCATGCCTCATTGTTGTTCTTTAATAACAAG
GAGCATGAGCTGAAGAAAATGCCAAAGGTGACATAGGAAACTGGGGGTGGGGTGGGGGACAAGAAAAACCTACTGAGTCAAGAGGCTTCCTTTC
CTCGCTGAGGACTTGCTGCTGCTTTCTACATTTATTAACAGCTCTTATGTCTAACCATGAGAATCAGAAGAAGATGACAGTTCTAACAGCTCCT
GCCCAAAGTCACTCACTGCATATAGTTCACACCTCAGGTGGCCTGGAATTCGCTCCTTAGGAAGAACCTGTAGCCAGAGGGTAACTTTCTGAAA
GTTGAGGTCAGAAATGCTTGCCTTACCAATTTACCTCTCAGGCCTTAAACTGTTTATTCATCTAGTCCTTTGTTAGATTGGTTGTACAGACAGG
CATGCTCAGTACAACCAGAGCAAGACATAAGAAAGCACACACAGGTGCATATACACAGCTATAATCTGGAGCAAGACATAAGAAAGCACACACA
GGTGCATATACACAGCTATAATGTGGAGCAACACATAAGAAAGCACACACAGGTGCATATACACAGCTATAATCTGCAGCAAGACATAAGAAAG
CACACACAGGTGCATATACACAGCTATAATGTGGAGCACCCTCTCTCGAGCTCAGGGTTAAAACAGTCCAAAGCTAGATTTTTGCGAGAACAGA
AATAATATTACAGAGACAGAACACTTTGGGCACAGAGACAGAAAAACTGAAAGTTATACCCAGAGAACATCAAGTAAAGATTTAACTAAGTTTA
GTAAAGTAACCAAAAAAGATGGGATAAACTGCTAAGTGTCCGGCGGTGCTTTAGCTTTAGCCATTAAACTAGAAAGAAATAACCAGGATCTGTT
AGCAGAGCCCAGAGACCTTCAAAGAGCTGAGAGCGGGGCAGAAGTCGAGATAACTGATCGTGGGAGAAAAGGGAGAAAGAAATCAAAAGCATCC
ATGGATCTGTGTAAAATTCCTCCACCAGGCGTCAGATGTGGTACATGCCTGTCATCCCAGTCCTTGAGAGGTGTAGGCAGCAAGAGCAGTTTCA
AGGCCAGTCTCAGACCACCTTATCTCAGAAAGCAAATCAGATTTGCTAACAGGACAGTTCATGACTGAAGCATGGTAGGTGATAACTATGGATC
ACAATACAGGATTTGGTACAGACCCAGGAGAGCCAACAGTGAAGAGATGCAGCAGAGGAGAAACCCAGTCAGCTCTGGTGCTTACTAACCACTC
CGCTTCAGACGTGCTGATGAAGTTTGTACTGTCGCTTCCTCATTTTTAAAGTGAGGGGAATAAATCTAGCTCTGCCTGCCTCAAAGCAACAGTC
TAATGAGTCCAGAGATGTGATAGCACTGAGTAAGCCAGACAACATGGTGTGGCGTTAAGTTACATTTAGAACAAAATAAAACAACCTTGGAGAA
CCACCTGTGCAATTCCAAGGAGGAGTAGAGAGCAAGAGGTAGCAGGCTAGCCCACAGGTAAACCGAATGCAAAGAACTTGTCTGGGACTAGGGC
GGGGGAGGGGGAGGGAGGAAAGGAGAGAAGAAAGAAGAGAGAGAGAGGCCAAGGACACTGACAATCTTAGACTAGAAAGAAGTCATCAGACAGC
TGGACTTAGAGTAGACCAAACGCTTATAACACATGCTTGTTAGTTAATACTGTGGTGAAAGGAGCAGGCTTGGGGAGGTAATCTTAGAACCATC
ACCTCTTCAGAAGTCCTGAGGGTGGGGGAAGTGGATGCTCAGCTTGGTGGCAGTTGAGGAAGTTCCTGCCCAGTGGCCTTGATATTTTCAGTAA
AAACTGTGAGAAGGAAATATGAGTAGGTGTGACCCGTGCAGACAGAGCGTTGTAACCAGGGAACCAGATGTGGCTCTAAGTAGAACTGACGGTG
TGGAGGCGAGGAGGCCTCTGATTTTTATGCCGGACTTTCTTACCTTCTGGTGCATATTTGGTACATTTCCCATCCTTTGGAAATTGCATTCACA
CTTGACTCTTTATATTTGTAATTGAATATTTTGGGAATGGTCATTTTTGAGCTCATGAAGGAAAGATACTTTAAACAGTCTCTACTGAAATCCT
TCGTTTGTAGGTAACCAGATCCAGCAGTTACAGCAGGAAGTCCCAGTTCCAGGTGGACGAGGAGGACATTCAGAAGCTGGAGATCAGGTCAGTG
CCGTCCTGTGGCGCTGGAGAACGGCTTTTCTCCATGCAAACCTCCATAGAGCCCTTCATAGCAAGCTGATCGATGAACTTAGATGAACAGGCAT
CTAACTTAATCCACTTAGCCTTTCAAAATCGGATTCTTCTCAGAAATATCAAGTAGTTCTCTTGTGTGAGCAAAGACAGCCTAATTTCACCTAG
TCTGTTTTCATTTTCCCTGATTTAATTGCTCCTTCACTACCTGGATTATAGTATAATGAATGGAAGACTGATTATATTTAATCCAATTTAGAAA
TAATTGTTTTATTCTTCTTAACATATCTGCTGACTATCAGACATTTCAGTGAGGTAAGGCGATACCCGCACAGGTGCATGTGTTTGGAGTTTGT
GGTATTTCCTAGGCCTTCAATATCTATGCTGTTTTGGAAAGGAAACTTTTGCAAATGCATGTCTAGGTGTGGTGTTAACAGTGTGTTTTCTAAA
AGGCATATTAATCATGTGTTCACATTGTTAGGCAATATTTTTCTAAATCACCGATCTTTATGTATTAATTAATAACATATTGATTTATCATCAA
AGAAAAAGTGTGGTCTTGTTTTGTTTCCTCAATTCAGTAAAGTAACCCTTTTGTCCTAATGCTACAAACTAAAAACAAACAAACAAAAAAAAAC
AACCCTAGACTTTCAAACTTAGTTTCTCCTTTTATACTGTTTAGAATTTGGCCAGGAAGCTGGCCTAGGTGGGAAAGGCACTTGCTGCCAAACA
AGTCTTCCTGAGTTGTATTCCTTGGACCCTCACAACGGAAGGAAAGTGCCAACTCCTGTTAATTGTCTCTGACCTCCACTGTGCACAGTGGCAT
GCATCCCTCCCCCAAAACTAGTTAATAAGGATGTAAATTACAATTTAAATCCTGATTTTTTTTTCATAAAAATTGCTGTAGTAATTTGCTTTCA
ATGTCTAGATAATGCATTATATGACAATGCTAATCTAACATCTCTTTTAAAAGCATGTAAAAATATAGTACAAATCAAAATCACTTGAAACTAC
TCAGTGTGGTTTGCCAAAACTAATGCTAGGTTTTCAATTTTCACGATTGACTTGTGGAATAATGAAAACCTAGTCCAAGATGTTTTCCTGGGAG
AGATTAAGGTTCCTGTCAATGTGTTAAGAAGTGATTCCTTTCATCAAGCCTGGTAAGAAGACAGATACTCTAATGAACCAAATTTAATTCTCTT
TAATAAAGAATATAAATATATGCTTGCTGGAAATAAGTAAAAAGTTACTGATATTAATTTGGTTATGAATTTGTTATTAGAGGAAAGATAAGTA
CTTTAATAGTTGTATCACTTGATCTGTGATACTGAGATAAAGAATTTAGTTGTCTAGGAATATTTTTCTGTTTAAGAATAAAATCTTGGGGCTG
TAGAAATAGCTGAACTCTCTTCTCAGTTTGGACGGGGTTTCAGATCCTAGCACCTACCTCAGATCTCTCAAACAAAGGGCTTTAACTTTAGGTC
TAAGGAATCCACTGGTCTCTTTGGTCTTGATGGGCACCTGTACATGGACACACACAAACACACACACACTAAAAATAAAGTCTTAAATCTAAGA
AATGAGAAACATTAAGACATCGAATGAACCGCTCGTTGATCCTTGATCACATGTCATCATCTGCAGTTTCGATGCTTTTCGGTCAAAGAATTGT
GGGTAGAAAGCAGAGGGCTTATTGAGAAGACACAGCAGTCTTCTGTCTGAGGCAGTGTATGATTTTCAGTTATCACACATTTACAAATGCAGAT
AACCTCGATGTGTTTTCTGAGTGTGGGGTATTGACTATAATGTCATTTGGCCTTTTCATGGCTCTAATAGTGATACTGAATTAATAACATTAAT
GTTAATAACCTTAATGTTTTGACATTAAATTCATACTTATTTCAATGTGAAACTTTTTCTCTATAGCCCTTAAAAACTAGTGTTTTCTGTCTCT
TCGTGTGTCTGAGCCCATGTTTCTTCCTAGATTGATTGGTATGTAGGATCATGCTTTCTCAGTTGTTGGTGCTTGTTGATGCTGCTATGTGAGC
ATGTAGGTATGCTTACACTCATGCGCACATAGAGAACAGACTTGACACTGGATATGTTCACCAGTCACTCTCAGTTTCTGAAGAGAGGACTTCA
GAACCACGAGCTCACTGATTAGCTAGGCTGGGTGGGCAGTGAGCTCCAGGGACTGCCTGTCTTTTCCTGCCCCCTCCAAGGATTGCAGATGTGC
ACACCACTAAACCCAGCTTTTACATGGGTGCTGGGAACCTAAACTCGGATTCTCATGCTCTGCAGAGCAAGCACTTTATCAGCTGAGCCATTTC
TCTGACCTCTGTCTCTTCCCTTTAAAGGTCACTTTTTAAAATGAAATGTAATATTTGTGCCTGCTTATTAAGTGTAATAGTCACTTGTATATAC
TGTGTAATGATCAAATCAGGATAGTCAGCATTTCCATCTCTCAAATACCATTTCCATGTTGGATGCTGGAGAATTAGACTGTGCTGAACCGTGA
CATCAGTTGTTACGGACCATAGTGAACCTGTTGGATTATAAACAGAGCTGCTACTCCCACGCTGCATTGAGAGCCCGTTCTAATAATAATACCT
TTCTCTGATGATCTCCCCTCCTGCCTCTAGCAACTTACAGTCTTTGTTTTTATGTGGATCAACTTTAGTTTTCACAAAGGAATAAGAACTTGTG
GCCTTTGTCTTTCTGTGCCTTACTTCACATGATAAAATGACCTCCAGTTCCAGATGGGTTACCATAAGTGATAGGATTTCACTCTTTAATAGCA
AACCGTCTGTTGATGAGTAAATGAATTTAAATAACGTGGTCTGTACACACTGGGATGTTAACATAAGAACCGCTCTTTAGAACTCAGCTGCCTA
GAAGAGGAGTTGGTGTTGACTGTTTAGGGGTTGGGTTTTTGTTTTTGTTTTTGTTTTTATTGTTTTAGTTTCTGAGACAGAATCTCACTATTTA
ACCTTGACTGGCCTAGATCTTACTATGAGGACCAGACTGTCCTTGAACTCAGACATCCACCTGTCTTTGCCTTCTAAGTGCTGAGATCTAAGCT
GTGCACACTCCAGCTGACCTAGGACTTAGACGTTTCTCTTTCCTCTCACTTGCCTTTAAAGGAGCCTGCCACCAGACAGTCCTCATTGGGATTC
TGCTATTCCATAGCCTTCCTTAGAGAGACTGTATTTTAGACTAATTTTCAAATTTTATTTTTTAACTCTTCCAATGCAAATCTAAATGTATTAG
TCTCTCTAAAAACATTTTGTGTGTTTTAATTTCTAAATGCATCCAGAGAACGAGAAAAATGACTTTAATTGTTGAGAAAAATGTATTACCGTTT
GCCTCCCCTGCCCCCATGTCATATTTCCTCCCTTCTCCATGAAAATTCCTGAACTCAGTTTTTAAGTCTTGAAAATAACATTCCATACCTTTAG
TACAGAAGAAAAAATGAGTTGTTGTGTCTCCCAGGGGCCTCCACAATGAGCCTCAGACCTGCCCCCACAGTGTGACACAGTCTTTCCATATACT
TTCAAATTTGACAGAACTTTCTACCTTGGCCAACTTGGAATATTAACAGACAATAAGTAACTTTATTTCAATTTCTTCTATCAATAGTATAAAT
ACTTCATGTGCTCTTGAGTGCTGGTGTCTTTGAGGTCAGAGTCGTTGAATCCTCTGGAGCTGGAGTCACAGGTGATTTGTGGCTGCCTGATTTT
GGTTGCTAAGAATTGAACTCGTTCTTTGTTTATGATCTCAACCATTGAGCTAGTTCTCTAGGCTCCAATAAAAATATTTTAATTTAAAGTAGTC
TGTCAGAAGCATAAATGCTAAAATTTATGTAGCATAAAACAGAAGAATCTGCTAATGTATATGAATTAATGTACATTGTATGGAAGTATAAAAA
TGTTAATGCCATTTTAATTGAAGATCTTGATCTCCTTTATCACTTATAGAGCTGTAGCATCCTCTAGTGGTATTAAAGATAACTGCACAAAACC
TCATTCCATTCAGGCATAGTTATTTCTAACCTTGATATTGCAAAGCGTCATAATAACTTATTAGTAGGCAGTACTCTTGCTTTAAGTTTTTCTA
TTTTCTTTTTTCCAGTAATTTCCATTAGTATTTGCCTATTATATTGTATGTCTAAAAACTGGTTTTTCATGGTTTAGTTTTTTTAAGATTTCTA
TTTTATGTATATGAGTGTTTTGCTCTATGTATGTCTGTGTACTGCATGTATGCCTGGTGCTTGTGGGGGTCATCAGATGGTGTCAGATGCCCTG
CCACAGGAATTAGAGATAGTTGTCAGCCAACATGTGGGTCCTCTACAGAAGCGGCAAGTGGTCTCCACTGCTGCATCATCTCTCCAGCTCTACT
TCCTATTTAATTTTATTTGCAGATTATAATACAATGACTCATGCCACTTTCCCTTGCTTTCTAAGACGTCTTTTGTTGCCCTTATGTACATATT
ACTTTTATGTGTGTCCCTTGCGCGCGCAGCCTGTGAATGCCTGCAGGAATTGTTCTCTGTTTCCACCACTTGCCTTCTGGGTATTGAAGTAAAG
GCATCTCTGTTTACCTTCAGTCGGTTCTTGGTGGTGCGGCAACCTGGGAGAATGAAACGGACTACAGTCTCGTGCTAGCCAGCCGCACTCAGAG
CATCACACATTTCATACTCGGAGTGATTTACCTCCTTAACCTTGATTGTTTTTTAATTGGTGATAATTAGATTGGACGTTAGCATTGTCTTTTT
TGTGTTGATATTAGCTAATTTTCATTTTGCCATGTGTAGATAATAGAACATAATTTGATTGTTTTTTTAAATAAGTTTTTTTTAATAGAAATTA
CACCATTCCTAGGTAAAAAGATGTAAAAGAGAATCCTGCCTCCTTCCCCAGTGCCACTTGCCTAGGTCCTATTTCTGAGCCAGCCTCTGTTCTC
TAGATTCTGGGAATATTTTCTATATTCATAGGCAAATGCATGTATATATTTTCATTCGTTGTTTTGTTGTATTGAACCTGGGTTTCTGTGTGGC
CTGGGAGTCATAATCTCCCAAGTGCACACACGACCACATGCCCACCTGTACTATGCCTGTATGTTTACTCAACAGTGTAGTAGAACACTATAGA
TAGGCGTTGACTCTTGATGGTGGTTTACAACTTATACTCTAGAATCAGACAGCCTTAATTCAGGTCCCATCCTCGCCAGTAACCACTTGCTCTG
TGCTTCTTATTTCGTGGGCACAGTCAGGGCTGAATGTGCTCAGAACACAGTCCGCCCATGACCTGCAGTCTGGCCCTGGCCCGTGGTTTCTTCT
TCAGTAGCTCAGCTGTCTACTTATCCCGCTTGCTCGTTAACTCACTACCATCACGTGTAGTACCATCAGCTGTACTTTTACTTTACGTACTTGC
TACAGCCAAGAGACAATGGAAACAAATCATCTAAAACCGATGACCTGGGATCTCTTCTGTTAACTCTCTGCTATACAGAAGACTGCGTGCTTCC
ATCAGAGTACTATGGACCGCTGAAAACTTTGCTGCTAAAGTCACCAGATGTTCAGGTACTTTAAAAATCTTTTTTTTTCCCCAAGTGTTTTGTG
ATTTGTTCTGGATTTGTATATAGGTACAAAGCTTCAAGGGATAAACCCAGAAAAACGGTCACCTTCATCCGTCCACATGTCGGAGGAGCCCTTG
TGGTCAGCTCTGGGAACTACTTTAAAAATCTTATATGATCCGCAAGTGGGGCAGTCTCTGGATGGTCGTTCCTTCAGTCTCTGCTCTGAACTTG
GTCAGACTGCCCCACTTCGGGATCCATGCCATAAACAAACCCAGGCACTATGGCAGATGCCAAAAAGAGCTTGCTGACACGACCCTGATATAGC
TCTCTCCTGAGAGGCTCTGCCAGTGCCTGGCAAATACAGAAGTGGATACTCACAGTCATCCATTCGATGGAGCACAGGATCCACAATGAAGGAG
CTAGAGAAAGTACCCAAGGAGCTAAAGGGGTTTTCAGCCCCATAGGAAGAGCAACAATATGAACTAACCAGTACCCCCAGAGCTCCCTGGGACT
AAGCCACCAATCAAAGAAAACACATGGTGGGACTCATCTCTCTAGCTGCATATGTAGCAGAGGATGGCCTAGTCAGTCATCAATGGGAGGAGAG
GCCCTAGGTCCTGTGAAGATTATATGCCCCAGTATAGGGGAATGCCAGAGCCAGAAATGGGAGTGGGTGGGTTGGTGAGCAGGGGGAGGGGGGA
GGGAATAGGGAATTTTTGGAAAGGAAACTAGCAAATTTCATTTGAAATTAAAATATCTAATAAAAAAAAAGAAATTAAAAAAATCTTACATGAA
AAGGCTAAACTAATTCTGCAAATGATAAAAGATTTCATAGTGTCAGCTCTTACAGAGTGCTGTGTATTTGATTTCTCTGAACCTGAATACTGAT
GTGCTTCAAATCTTATCTCTGATTATAATTTAGAAAACTAAATACTATCTTACCTCTGATCAGTTATAATTAATGAGTGTGTTTAAATTTCATC
CACAGGTTAGTGATTACGCATGCAACCATGGTTCCTTGATTCTGTCCTTGTTTTTACCTTCTTTATCCTAGCCAGTATCCGCCTCAGCTGCTTA
TATTTTGGGTGAAATATGTCAAGATCAAAAGGATGCTGTTTTGCCCCTTGTACGACTGTTCCTGCATCACAATAAACTTGTCCCTTTTATCACT
GCGGTGGCCGAGCTGGACTTGAAGGATACTCCGTAAGAACTGTATTTCATTAAATGTTTAAACTTCAATTTGCCAGATTGTTTTTATAGTCTCA
GTTCTTTTGTATTGACCAAGTTTACTATACACTAGGTTAAAAAGAATCGCTTAGTCAGTAACAAAAGACAGTTCTTAGAGTTACCAAACAGAAT
CCGGCTGACCGTAAGAACCAGTTATGCAGACATGTGTATAAGTCAGTGGTGCCATTTCCTTTGTTACTATTACTGATCTTTGCTCCTGGATGCA
GTGATTCACATGTCACAGCAACAGCTCTCTATCACCCTTGTTACACTGTACGCACACGTAGAGCGTTGTGAAGAGCGGTCAGTGCCTTTCCATT
TCCATCGCAGTGAGACAACACCATCATCTTATGCACACAGACCCACACTACACCATCATCTTATGCACACAGACCCACACTACACCATCATCTT
ATGCACACAGACCCACACTACACCATCATCTTATGCACACAGACCCACACTACACCATCATCTTATGCACACAGACCCACACTACACCATCATC
TTATGCACACAGACCACACTACACCATCATCTTATGCACACAGACCCACACTACACCATCATCTTATGCACACACACCCACACTACACCATCAT
CTTATGCACACAGACCCACACTACACCATCATCTTATGCACACAGACCCACACTACATTTTTTTTACATTCATAAACATAGTTTTGATAGGTTT
AGAAATTCTAATGCCAGCAAACATTGCTGTTAATAGTAGTAAAGGTTACTAAAATTGTGTATATGTTCAAAGTCAAATTATTACTGTTTGAAAC
TTTTCTTCTTTTTGCTGTCAATGGTTTTTGTCTCTTTCTATTTCACAGATGCCAACGCAATATTTAGAGGAAACTCCCTGGCTACCCAGTGTCT
GACTGAGATGATGAAGATAGTGGGAGGGCACTACCTGAAAGTCACGCTGAAGCCCGTTCTAGATGAGGTACAGAGTAGACCTCACAGGCAGAGC
CTGTGACCAGCTAGAGCAGCCTTCTCAACTTGTATGTCCTGAAAAGCCCTTTCACAGATCATCTGAGGCGTCTGCGTGTCAGATGTTTACGTCA
TGATTCGTAACCACAGCTAAATACAGTTATGAACTAACAGAAAGAGTAACCTTACGCAGGGGTCACCCCAACATGCGGAGCTGTAGGAGAGGGC
CCCAGCAGTAGGAAGGCTGAGAGCCACTGCAGTAGACCTTCTCTTTTAAGAAGTAATGTGTTGATTAAAAGGGTCTGTCCATTCATGGGCTACT
GGAGGAATAGGTGTAGACACTTTGTAAGTAAATTTCTTTGCCCTCTGTGGATTCATTCTTACTCACAAGACACACTGATCTACATTATTGTTTG
TTAGATTTTTTTTTAACTAGGATTTTGATATAGTTCTTTGGGAAATTGTTTATGCTGCTTTAAGTTATCTTCAGGTCGAAGCTTTAAATTTTGT
TAGATTTATTTTTTATAATATCAGAAGAAGAAGAATTATATTCATGTTGTTAAATCACCTAGGAAAGAAGAGTAACTGAGAAATACTTGTATAT
TCCAGATATGTGAATCCTCGAAATCCTGTGAAATAGATCCTGTTAAGTTGAAAGAGGGAGATAATGTAGAGAATAATAAGGTATGATTATACAG
TGTCATTTATATCTACATTAATTTTTATCTTCATGTGATACCTGTCTCCCATAAATGGTTTATTTTATTTTCTATGTATGAGTGTTTTGTCTGC
ATGTATGTCAGTGTGCCATATGTGTGCCTGGTACCTGTGGAAGTCAGAACACCGCATTGAATCATCTGGGAATAGAGTTCAGGTGGTTATAAAC
CATTATGTGGGTGCTAAAATCAAACCCAGGTCCTCTGCAAGCACATAAGTGCTGTTAACCACTTAGCCACCTCTCTACCCTCCAACATATACTC
TTAAACTGTGCTTCTTGTGAAAATTACTCATATATTCATTCATTTGTCTAGAAAGTCTGAATTTAACTTAATAAATTAACCTTAATGTTGATCG
GCCTTTAACCTGAGTTCGGACTAGAAGCAGGAGACTCGAGAGTCATCTTCTAATAAGTCTCATACCCATTATTTGTTCTCATGAACATATTGAA
GCTGTGTGACAATCAGAATCTTAGTGTTTACTTTGTTAAAACCTGCAGGAGAATCTGTACTACTATGTGGACAAAGTATTCAATACAATCGTAG
GATCAAGTGTGAGCTCCCCCACTGTGATGTGTGATATTTTTTATTCTCTGAGCCAAATCGCTGCTAAGAAATTTCCTAGTAAGTGCCTTGGTAT
ACTTAATCACATTTTCTTTTTCTATTTTTTTCTCCATGCTCCTCATTTGGTTTGTGTGAAATACTTGTGCATAGGTACAATCTTCTGAGTTTTT
CAAAATGGTGCTAGCTTCTTTGTGAGATTTAATGATTTATTTTTTCTAGAATCTCAAAACATGCAATTCACTTTATTTCAATATAATGGCAACT
ATATAAAGAGCTAATCAAAATCATTAGTTTTAAGAAGGGACAGTTTGCATCTGTACAGTTTTCTATAATATGTAAGGAAATCTTTATGTGTCGT
ATAGTTAACAGATTGTAGAATAAACCCGTGTGTACCAGCTATTTTTTCCAGTTGCAGATTTTCAAACAGTAGTAATTGGTAGTTTTATACAATT
GACTAGATTTGACAGCTTGAAGATACTAAGATGTCTGTGGTGACATGTCTTTTCAGAAATAGTCTTTGGCTTTACAAATACTTGTCCCACAATC
CATAGGATGATCTTGCTTCAGGTTTTGCAAAAATATCTATATTAAGATTTTGCTTGGAAAGGCTTTATAAAGTTATGAGATCTACTTCTTATTG
TTAGCACTTGGAAATACTCAAAATTTTAGTTCATTTCTGTTACATAAATCAATTTGCCAAAACTCATTGCAGATCTGCCCATAACACAGACAAG
GCAACTATTTACAATAATAATCAGGTTTATCTATCTGCATAAGACTGTAAATTAACTTTCTTAGCCATCCAACGAGATTTTGTTATTTCATTAT
GCTTCCATTCGTCAGACCCTTTATTTAATTCACTCATACTAACTGCTACATGAATTCGGGTTTTGGTCTCTTGGCTAATTACAATAAAGAGATA
AAACATTTAGCTTTAAATAAATATATCTAGTCTTCATAATCAATAAAGGAGGCAGAGACTTGATTTTGCATACATAAAGATGAAATAGCAATTA
GATTCTGTTTTCAAATAGTGAGCTAGATCATAACCACTTTCCCTGCTTGCTAGTCTGTCTTATCCTTTACTCTTAACATGTTTGAAACCATTAC
CCATTGATTAGCCAATGAAACTGAGGTCTCAGTTGTTGTTACTACCTGGACCTCTGCTACCCTGCTAGCAGGGCAGGCAGTCATAATGATGGAC
AGCTTGACAGTTCCCTACTGGGTGATTTGTAAGACTAAACCCCACGTCAAATCCGGAGATTCTTTCCCTCACTGCCCTTTCTCTGGCGTCTGAT
TGTTGGGTTAGTTGTTGACTCCAGTCTGAAACAAGTCCTGACAATGAGTGATCATTGCTACAACTAGCTTTCAATTCACCTGCAGTTTATGTGA
AATATTGGTAGCTTTAAAAGTACTGTCTCTACCTAATTGCTTTTAGTCTTTAAATTGTTGGCTTTTCTTTTTGTGACAAGTACATGAGAGAAAC
AACATAAAAGGTAATTGTGGGTTCATAGTTTCAGAAGCCTCACTCCAACACCCACAGGCTCCGTTACTTTAGTCAGGCCTGAGACAAGGCAAAG
CACTGTTACGGCATCTTCTACCAGAGACCGCTTGTCTCCTGGCAGTGAGGAGGCAGACAGAGGGAGGGATGAGCCAGGGCCAGAATACACCCTT
CAAAGACATATCCAAAGTCCTCTGCTTCCGCTAATAAGCCTCCAGCTTCCACAGCCCCACCACCTCGTACAACAGTCTATTGCAGTTGGAATCC
GTCAGTGTGGTGAACGTTGATTACATCGTGATCTAGTCATCTCTGGAAACATTCTTACAGAAATACTCCAGATGGTGGTCTACTGGTCTTGTAG
GCATTTTTCATCTAATGAACTGAATAATCAAGACTAACCATAATATAGCCTTAGACACCATGAAGATTTTTAGGGTGAATTTAAAACAAAAATA
TAAAACTGAAAGCTAGTTTTTGGTATTCAGTGTTGTCACTAGGTAGTACTACGTAGGGCTTTAATGTTACTTTTTTTTAAAATAATTTCAAGAA
TCTTTTATATCAAGAAGCTATTTTTCAAATTAAAAGTATTTCTTTAAAAACAGCAACTTTGCTTTCCGTTTGTTATTTTAAGTTACAGCATTTT
TTTCTCTTAGTTTAGCCACAGTCAGCATCATTTCACAGCTCAGTTTTTACAAGTTATTTTCCTTTCCTACATAGTTTACATGATTTTTTTTTTT
GCTTTCTCCTCTTTGGTTTATAAGTAGTGTGTTTGCATGACGTTTCCCATTTTATCCCTGCAGATCACCCTCATGTTCAGTACTCTGCAGTGAG
CAGCTTTGTCTTCCTTCGTTTCTTTGCTGTAGCCATACTCTCACCTCATGCTTTTCATTTGCGACCTCATTATCCAGTAAGTGTTTGCTCCTCT
CAAAGCCTTGTTCCATTTTTATTTATTTTTTGTAAAATGAAGAAGAAATATCCCATTTTTCTTTTTCGGTGATAACACATTTCACAGTTGGCAG
AATGCTGCTAAGTTGATGTCTCCTGATGAGCAGAGGGGAGCACTCCTCAGGGCCTTTCTCCGGCTGCTGCACACGGGCTGCGGTCCTTAGTTAC
AGGGAGCTGGGGATGCTGGGGTGGTAGGTGGAGTTGGGGCTGGTTGTTAGCCGACCCTCTCCTGGTTTATTACAGCAGCTTATTTTTTATTTCT
ATATTCTGAGCCAGACTCTTGTTCCATAGCCCAGATCGACCCTGACTCACAGTCTTCCTGCCTCAGCCTCTAGAGTCCAGGAGTACAGGCATTC
CCACCGCTCCTGGTTTACGGTGGTTTAATCATGGCAGTATTTTAAGATCTCAGAAATGTGAAGGAAAATGAAGAAGTTTATTTGAAAAGTATTC
AGGAACCTGACTTACTTGGTATCTTTGTGCTTTTGCTTTCTGTTCACCTAAGTTATATCAAAAGATGAGACTAAAATGAAGTTCTGTTCAGTTA
CCTAAATCTACATCTGCGTTGAATATATACCTCTTCATATTTTAAGGTTTCCTTTTTCTTTTAACCTATTTCTAGTCCTTCATATAGCATATTT
TTAGTCACTCTTTGGTCTTTCTCTGTTGTAAATATTTTATATATACTCTTGATGCTTTAGCTGCTGCTGTGTACACTAGAATAGGAAACATTTA
GTGATGACCTTGTTCTAAGCTTTCCTTGAATCACTTTCTATGTCTAACTCGCTTAGGTCTTCCTAGTCACTGTAGGAGTAAATTACTTTATTAG
CCCCATATACAGGTAAGGAAATCACCATGCAATAACTTCTCTAGGCTCATATAACTAAGACATCACATTTGGATCCTACACAATTTGTACTAGA
TTCAAATGGGACTAGTTCAGTCTCCACAGTCTATAAGCAGACTGGTATTGAAAATCTTTTTTTTTTTTTTTTTAATGTATGTGAGTACACTGTT
GCTGTCTTCAGACACACCAGAAGAGGGCATCAGATACCATTACAGATGGTTGTGAGCCATCATGTAGTTGCTGGGAATTGAACTCAGGACCTCT
GGAAGAGTAGTCAGTGCTCTTAACCGCTGAGCCATCTCTCCAGCCCTGGTGTTGAAAATCTTACAGCAACCACTGTCACAGTGCTCAGTGCTGG
AGACATATGCACCCTTAATTGACATTCCCTACTTCAATGTACCCATGGTCCATCTAGTAGTGCAGTGTAGGGAAAACTTAGGGGCAAAACTGGC
TTCTGAATGTTCACTTCCCCTCCGAATAGCCTCACAGTGTTAGCTGAGAGCCATGCTGCAGTGGCAGAAGATGGATGTTCTGTGTAGCCTGTAT
CCTTTTGTTCATGGAGAACCCTTCTGTGGCTGTGTTAACTAAAGCTGGAATGACCTGGGGCAGTGAGAGCTTGCCAGCCACATTCTATACAGAA
GTAGAACTCCCACCTCTCCACTTGTGGCTCTGCTTGTATTCAGACTTGGAATTCCTAGATGGAATTTCAAGAAAATCCCTCTAACACAAACACT
TTTAGGTCACTTTTCACGCAACTTTATTATTTAAAGTTTTCTTTTTGTTGTTATTTGCCCCCATCCCCAATACCTCAGCATCTCCATCCAGTCT
TTAGCCATAATTCTTCATTTTAAAGAAAATTTGAAGTAGTGGGAATAGACACAATATGACATTTGACATTGCTGCTGTAGGGATGAGCATAGCT
GCCTTCTATTAAGGTAGAACTATTGCATCAGTTTAAAGCCAGCACATGATTAGCCAAGTAAAAAGTAATAGTAAGCACTGCTGTACAGGCCGAG
AATACAGAGCAATCCCAGACTCTGATGGCTTCATCAGGAACTGGAAGTTACAAAGTAATACAGGTGCAGAATTGATCAAATGCTACTGAATTTT
ATGTTTAGAAATCCTTTTAATTCTAAGAAACTAATTATAGAAGATGTTTTAGAGTAGACAAGAAAGAATAAAGAACAGCTAGCTAAATAATCAT
AGCAAGGGCAGGAGATCTGAGCGGTGCTTGCTGACAGTCAAGGGATGTGTGCACACACATGTATAAAAACTGTTTTGAAAACATTAGTTTAGTA
AAAATGAAGGCATGGGCTGGACAGTTGGCTCCGCGGTTAACAGCATTAACTGTACTTCCAGAGGTCCTGAGTTCAATTCCCAGCAACCACATGG
TGGCTCACAACCATCTGTAATGAGATCACATCCCCTCTACTGGTGTGTCTGACAACAGCAACAGTGCTCACATACACAAAATAAATAAATCTTA
AGAAAGAAAGAAAAGAAGAAGAAAGGGTGAGCTGGATTGATCGCTCAGCAGTTAAGAAGGCATACTGCTCTTTCTAAGCCCCTAACTTCAGTTC
CCAGCACCACGTCAGGCAGCTCACGGCCCTCAGTAACACCTATTCTAGACGGTCTGACCCCTCTTCTGAGCTCTGGGGACGTGCACCCTGTGTG
CACATACCTACACACAGATACACATATGCAGATAAAAAATAAGGTTTTAAAATAATGTAAATAATCAGATGGACTAACGGCTCCGACTAGAGCT
CAATGTTCAGTCATTTGCCAAGCATTTGTGACCCCCTACATTCAATTCCTAGGGCTGTAAGAAAGCAAAAAAAACGGAAACCTTCACTTGAACC
TCCTTGGTGATTGTTCTGTGTTTTTTTTTTAAATACAGATTATGTAAACATTCATTTTGTAAACCAATGTTGAAATTTCAAAACGAAAATCATT
AGATGATTCTTATTAAATCCCTCATTCTTCCTTGATTATCCCTTTGAAGGCCCTCCTGCTGAAGACCTCTTTTACCCATTGGTTTACTTTCTGT
CTGTTGTATATACCTGTAATACCCTGTGCACTCTCTCTCCCTCTCATGGCATTTTCTATTCTGGCTAGTAACATCTGTGTATAACAGATCTCTG
CATACAGACTGCCAGCATCTCCAGAACAAGGGAGGACCTTTTCCTAATATGTGTATCCAAAGCATGTGGTAGTGTCTAAGACAGTCACATGATC
AGTTTAATTAATAAAAATGATAGTTGAGAGAAGGAACTCATCAAGTGAGTCTTTTCTTAATGGAGAAACAGATTCATGGTTTAAATACAGGGGA
AGAGGCAGGCACTCGAAAAGAGGGCTTGCTGCCTCCAGGTCAGAATCCTAGAGGATTCTAAGCAAAGGTGTAAGATTAGAACCGAGCAGCTGCT
CTTTTGAAAGATAACAGCAAACTTGGGACATTAGTTTTTTTGTAGACCAAAGACAGTCAAATGGCACCAATGAAAAACAAGAGAATGTGTGCTG
AGTTACTTTGAATTAAAAAGTTTAACATTTTGGTTGACTTTAATAATTGTTACATAGTGTTTGCTGAGTTGTGTACTGGAAGCCAGGTGAGGAA
GGTCCTGGGTATACAGTGATGGGAAATTTTTATTACAGTTTGCATGTATACAGTAACGAGTCAAAGCAGCCGAGTGACGTGGTACTACAGTAGC
CACGGTTATGTGGGTCAAAGCAGCCGAGTGACATGGTACTACAGTAGCCACAGTTATATGGGTCTAAGCCAGCCTTGGTGACCTGACACTGAAT
AAAATGAGCTCTGTTCCCTCTTGCTACTTCAAAAAAAGAATAACCTAAAACACTGAATTTAAAAACCACGTTTAAGCCCGGCAGTGCTGACACA
CACCTTTAATTTCGGCACTGAGGAGGCACAGGTAGGCAGATCTGTGTTCAGGGCCAGAATGGTCTACAGAAAGAGTTCCAGGACAGCCAGGGCT
ACACTGAGAAACCTTGTTTCAAAAAATCAAGAAAACAAACAAATCAACAAGTTTAAATGGAGAAGTCTTGTGAAATTCAATTCAAACTTAATGT
TCCCGGAACTTCTAAAGAATGTTAACTCTCTTCCCAGGTAGAGCTCTAGTGGGGTTCTCTTACAGGTAGAACAGAGTCCCAGGTGCTCCATCTG
AAGTCATTTTTAGATAACCATAATTTATAACTTTTACCAGATATATCATAATGGTCATATACCAGATATTCCTTCTCCGGATTTCCTCATTTCT
TGGTCATTTCTTCCCCACTACATTCATTGCCTGCTTTTAATATGAGATATTGAAAGATAAAACAGTATTAACTATAATAATTGTATTTTCCTTG
ATATGTCTTTTGATAATATTGTTTACTGTGGTAAGATAGTTCGTTATGTAGACATGTAGGTCAGTAAGACTCATCAGCGAACTGTGTTCTGTCC
ATTTATTATATGTACTTTAATGTAAATTACACTACTATCAGGTAGACAGACCTGAAAGATAACGTGGCTAACAGACCCAAACAGTTACCTTCCA
GTACGTTCTTATGTGAGTGGGTGACTCCATTTCCTGGTCTGAATTTGTATTCTCCCTCTGCTCCCATCATGGGAACAGTTGATTGCAGAAGTCT
TACGTGTTCTTTGAATATGCTTAGAAGTAGACTGGGCACAGAAAGTTCGAAACACCTCTAAGTTAGAAAAAAAAAATAGGTATCTCTGATATTC
CAAAAATAAATAGATTTGGAGTGTGATCCAGTCTTTCGACTACACAAAGCCCTTAGATACAGTTGATTACATTATGTTTTTCTTTTTTTCTAAG
TCCTTAGTATTGTCTTTAAAGATTGATTTATTTATGTTATGTGTATGAGTACCACATTATGTTTTTCAAAGGGAACAACTCATTAAGTGTTTAA
TCTCTGACTTTTTTTTAATGGTACTGAGAATGGAATTGAAAGGTCACCCATGCTAGGCAAGCATTCCACCACTGAGCTGTTCTACCGGCCCTCT
TTACTCTTTATACTGAGGCAAAGCAGTCTCATTCGGTTGCCTGGACTGGATTTTCTCTGTAGCTTTAACAGACCTGAGTTTGCAGTCCTTCTGT
CTGAGCCTCCCAACCAATTGAGATCGTAGACCTACACCACCAACTTCTTTCATTCTCTCCCAACCCAAGTTCTATAACTTGTCTTAGGCTTGGC
TGCATCTTAACTATCTTTGTTTGAATGAACATTTTTAGCCATTATTTTAGAGCCATATAGCCCATTCACTCTATTCTGCTTTTTATATAAACTC
AACTTCTTGAAAACTTAAGAATTTTGATCATTCTTACCTTTTCTAGGATACACAGACAGTTAGGACATTGACTCTCATCTCAAAAACCATTCAG
ATTATTGGAAACTGGGGATGTCAGTCCAGAAAAAAGGTGAGTTTATAACTGTTTCCTCTTTCCATACTTAACAAATTTTATTATATACTAAGAT
TCCTTCTTCACTTACTTCCTTACATGTTAAAAATAAAACTGAAGAGAATACATATTCACATGCAATAATGGTCTTTTAGTGGGTGTTCTACTGT
AGTCACCTTTTGAAGGAGACGGTGATACTTAACCTGATATACCCGTGGTAGGGAAAAAACTAGGAAGAAGACACAGGCACAGCTCAGTTATGTG
GTGGTCTGTGCCATAGTCACCTAACAGCACTGGAAGAAGCCTTCAGCTCCAGGATGGATGATTCATGGAGACATGGACAGCTTTATCCTTAGCT
TCAAAATACAGGCCCGTCATTGTCTTGTGTTCTACCTCAGAGCTTCATAAACTTGTCACTCATGAAGCCGTTTACTTAAGAAACTTATGCAACC
TTACGTATAAAAATCAAACATTTACTGATAATAACTTACTTTAGCATTTTGAAGTCCTCAGCTCCCAGTGCTGAGGAGTTTTTTCTTCCATTTG
CTTTTGCAACTGCTTTCTTGGGGCTTTTCCCTAGTTTAAGACCTAGAAGGAAAAGGTCAAACAGTGACTGCCAGTTCAAAGCTATATGCAGGCT
CTGAGGGATATTCACTGCTCCTCGGTTCCTCTCTCACCAAGAATGTTCATCAAAGAAGGGAGAACAGCCAGGCTCAGATCAATAAAGTAACTGC
AGGTCACTGACTCTACAAAAAAAAAAAAAAGGCTTTGCTGGCACCCTGCATCTGGCCCAGCATGAGGTATCTTTGGCTATCAGCAGCTCGAATC
AACAGAATGTGAATGGAGCCTCATCATGTTCAGGGAGAGGCTGAGCATGATATCCCATGTGCTGTGTGGGGAAGGCTAAAGACCATGAGTGTGT
GGTAGCCATGATCCTGGAACATTCTGGGTTCGCTGACTTGGAGGAATGCTCTCTAGGAGAGCTCTGAACAAGATGGTGAATGTGGCTGTGAGTT
CCCAGCCCGCCTGCTGTCCCACATCGAGAAGAGTTGAAGAAGCTCTCTATAGTTGCTTCTGAGCCACGATCTGGCCTACTACACAGATGAGGCA
GCACTCTTGGCCCTCTGGCTGCAGACCTGGCTCCTTAAGGCACCCAAGTGAATTGCCATGACTAATTATGACTGATTAGCCAAGAGGAGAGACA
CCACCTTACTTCCTACCTGACATGAATGAAATCTCCGCATCCCAGACGATTGGGGAACCAGAAGAGATTGTGCCGCGCTTTCCCGGAGTTGGCT
CTTATCAGTGGGGAGAACCGCATAGTGGCCACCTACTTCCCTACTCTGAAGAAAGTAGACTTGAGCTCATCTGGGGAAACAAAGCAGTTGGTAT
GGAAGCTGGAAGCCTTAAGAGGAAGTACTTTGCCTTGCCCTGGAAACCTGCCTAGGACACGGCTGCTATGAAGAGCCAACAAACACACAAAGAA
AAGCTAGGATTGGGTGACCTACACTTCCTCTGATCTACTAACTAACCTGTATTAGGTACCTTTATTATGTATAACAAGAGGAGTTAGCTGATCT
TAATGGGAAGTCTCTGTAGGACACAGTCTCAGGCAGTACTTCCTGAAGGAGGAAGTTGTATTTGCTAGTTTCTGGTCCCAGTAAATGGAAGGTT
TTGCCATTTACAGGGGTCTCAGGCAACTACAGTCCTTGACAGGGCTTTGTTCTTGCCATCAGTCCACATTCACCCTGTGCTTCATCTGATCCCC
ACACTAGAGTGTTGTAAGTTCCAGGTTCTATAGGGAAAATTTATGAAGTTGTTCACAATTTTTCATTTGTGATAAATACATTTGAACACTCCTA
AGCTGGGCCCATTCCTGTGTGGCTGGTGGGAGCAGCCCCTGTGACCTGGAGGCCTCACCCTTATCACTGTCTCTCACCTCTCAGGATCTTTCTT
TCCCTCCCTGCCTTCTTCCTTTCCCTTGAGACTAGAAATCAAAGGGCTGGGCTGTATCTACCTCAGTGGGAGAGCCCTTATCTTTTATAAATCA
GACCATAGGTTCAGTCCCCACTACTACCATTTTTAAAACAAAAAAGAAACCAGACATTGGCCTGCATAGCTTCCATCCAGATCTGCATCCCTCC
CTGTGTTTACTCTGAGCCTCTGGAGAGCCATGAGCTTCCAGGAGGCCCTTGTTTTGGATTCACAGTTGCACTTGTCAGGAGACCCCCAGGTAGA
TTTTTATTTCTTACTTGGTCCTTTAGACCTTGCAGTAATGATGGCTCTATACCTTATTGTATGCTAGTTAAACCTATCCATGCATTTGTAAGTA
CTTTTCAAATTCTCTGTTACATTTTTCATTTTGTCATATAATAATAATACAAATTTATATATGCAATGTTTTAATACATATTTACATTGTATAT
TGGTCAGATATTTTAAACCACTCTTTGTATATATAATTTTACCACTTATTAAAGACAAAATACAAATTTGCATACTAACGAGAACGATATACTT
GTTTATGTTTACATAAATAAATACTAGTTGAATATTTAGAGTTGAGTATATAGAGCATCTTCAGTGTTTTTCAGAGTTGATTGTATTTTGATTT
TGTAATCATTCAGTTTACATAAACATATAGTACAAAATGGCAGAAGTATATTTAAGGCCATTTTTTAAATTGTTGGAAATTTTGTCCTAATCTC
AAGCTAGAATTTACTTAGTGTTTCTTTGGTTTTTGGTTGGTTGGTTTGGGATTTATTTGTTTGTTTAGTGAAACTTAAGTCAGCAACATAAGCA
ATTAAAGTCAAACATTCTAATAAAATCCAAAACTACTAATTGATACATGCTGACTAATGATGTGGGAGAATATTATAGGTTTTTGTTTCTGTAA
TTAAGCCATTATGTTTTTGTTAGAGCAAAAGCCTTCATGTCTAGTAAAAATAGCTCAGTTCCTAGTATACGGTGGCCCAGTCCTTTGCGCTGAG
GCCTTCAGGGCAGCAGTGACTGGGGATGTGTGATGTATTGGCATGCAGAGTGCAAAGCATGCCTGCGGTACCATGCAGCAGAAACACCTCAGAC
TTGGACATGTTTGATTTTGAACTAGTTCTTGTTGTTTCTGTCTTTAGAAACCTTTATTCTTCATAAGTTTCTCATGACCACATGTGAGACACGA
CTAATAATTTAAGAACTGTGCGTATTTAACATACACAAATCCTGTATTTGTTTTCTCATAGTCACGGTTCAAAAAGTCAGTCATGTGTGAATTT
CTCAAAATGTTTCAACAAGAAAGATATTTTACAGATGTCAAAAAGGTATCCTTCTTTCTTGTCATCTAGTTTAAAGAAATCAACATCTCATATT
CTTCCAAAGAGTCCATTTCCAGCTTTAAAAAAATAAAATCATGTTGAAACTGTCAACCATGAAGTCTTTTTTGGGTTGGAGAGATCGCTTAGAG
GTTTAAAGCACTGACTGCTCTTCCAGAGGTCCTGAGTTCAATTCCCAGCAACCACATGGTGACTCACAACCATCTGTAATGGGATCCGATGCCC
TCTTTTGGCACCTAGGGCATGTATGCTGGCAGAACAGTGTGTACATAATAAGTAAAGAAATCTTTTTTTTTTTTTAAAGGTCTTTTTCTTCCAA
TTCTATCAAATAAAGCTGTGAGATTAAGCAAGTCTTAACAATAACTACTGTAAGCCTTAACTCTATAAATACAGATTTCTATGCAACACTATTG
ACATTTGGGGCTAGGTAGTTATTGATTTTGCATCGTAGGATATATGGCAACATCCATCAATTAAATGTCAATGGCATATACCCAGTTTTGAAGG
CCAGAAATGCTTTTAGCTATTACTAAATAGATGTCCCCTGGAGGATGGAACTGGCTTTGTTAAGAACCTTTGTTCTAGGACTAAAGCCTAGTAG
AGCTTGTTATGTAGTAGAGTGGGTACTCACTTGCCATAGCAAAGCAAAATCGGTCACCAGGATGGCTCAGCAGGTAAAGGGACTTCTTAGAGGA
CCTTATTGCTGGGGTCACAGTGATTGGAAGGAGGGTACCAGCTCCCATAGTTGTCCTCCAACCTCCATGTGTGTGCTGAGACACACACATTCAC
ATAAAGTCAGAAAAAAATGTAAAACAGTGTTTTCAATATAATGAGTATGCATGGTAGTAACTGGAAAAAAATAAGTAAAAATAAAAATATATAA
ATGCCTACAGCATAAAATGTCGATATAAGACCTACAATATGTAAAAAGGAGTGTCACTAGAAACTGAGAAGTCTTTTTAAGTCTTTAATTTTTT
GGTCCTTAACTTGTTATTCATTCACATAAAATACATTATTATAACCCTGAAAAACTTGGAACAAAAAATTATCTTAGGATATAGCTAAACTAAA
CTTGAAGAAAGTAATGTTTCGTTATCACAATCCTAACTTAGTGAACCAAGTTAGCAACCCTAGTCTTAAAGCCTGTTGAGTTTCTATACATGAT
TTAGCTGTTGATGGCCAAGAACATTTTTACTGAAGTACCTGTTTAGACTGAAGACATACTTTCTCGAAGACTCTATGAGTGAAAATGCTCCCAT
CAGATAGGGCTTGGTTTTCTTTTATTTTTACAGAGTCTCACTATATAGCCTTGGCTATCCTAGTTCTTAGTATGTAGACCCGGCTGGCCTCAAA
CTCACAGGTAGCCACCTCCCTCTACCTCCTAAGTGCTCAAACTAATCGTGTACACCACTATGCTGAACCTAAATTTTTTTCTCCTTTAGTTGAA
AGCTTTTGAGAAAATTTAATAATATGCTTTTAATTTGTAAAATAAAATTACAAGTTTTGTATANNNNNNNNNNNNNNNNNNNNNNNNNNNNNNN
NNNNNNNNNNNNNNNNNNNNNNNNTACATTTACTGTCTCATGTATATCTGTAGTCCTGAGGTAGCATAAGATAGTTTGTCCTAAAATCTTAATT
ATCGCCAGGCAGTGGTGGCACACGCCTTTAGTCCCAGCACTTGGGAGGCAGAGGCAGGCAGATTTCTGAGTTCGAGGCCAGCCTGGTCTACAGA
GTGAGTTCCAGGACAGTCAGGGCTACACAGAGGAAACCCTGTCTCCGGGGGAAAAAAAATCTTGATTATCTTTGTGTGTGTCCATGTGTCTTTC
TGTCTGTCTATAGATCGGACCTAGGATTTTACATATCCTACACTGTTCTGTCTCCAGCCTCTATAATTAAGGAATTACGAGTGTCCATGTAACT
CATCCCATACTGGCATTTATACTAGTGACTTTTAAGTGACTCGTAACTAAATCATACGCTGTTGCCCACACAGTAAAAGGAAAGGTAACCAGCA
GAGTTCTGGATACAAGGTGGTTATGCAGTTTGGTGACAGAAGCACAGCACTGTGAGGGAGTGAAGATGTTTACAGCTCTAAGTAGAGTGAATCT
AAACCAGCTCCTTACCTACAGTTACTTACGTCTGAGTGGTTTCACATCACACATTGGCTTTTATCTGATACTTCTTCATATAATGCTAAGAAGT
GAGATATTCAAGTAGCCATTAAACTCCACTGGTGGATGGCCCATCTATTAGAACTACAGATGAGACAGCATACTTTGTTACTGTTGTCCCTGGA
TACTTGAACCTGCACCACCAAGACAAAAAGGATTCTGTTCTTGGTTGTTCACTTACCAACATTCCCTTCCAGCAAACTCGAAGGAAGCCTCTTG
AGTGAGTGCTCTGGTGGGCGAATCACAGCACCCATAGCCCCAGCTTGCTGCGCCTTCAGTCCTGTAGAGGAAGCACTCGTCCCTTGATGTCCTT
CTTATAAAACTATGTGCTTTTGTCGTGTGTATCCTAAATCCTAAACAAAATAAAGTTAAAAACAAAACCGAAGACAGCAATACAGGCCAGACAA
CTGGGAGCATAATATCTCTTGGATAAGCAGATAGATAGGATTCCTCTCTAAGACCGGGAGGTGCTTTGTGAAGTGCTGTTTCCTCTTTCCACGT
GCTGGGAAGCCCGTGACTAGTCCAGCTTACATACTCATGAAGCTAAATGCTGTTCACTGAGTGGCCTCCTGTTTCTAGCAATGGATGCTACAAA
TTTTCTTGAGCCACATCCTTATATTCTGCATAGAAAATTTGTAAATATTTATATTTGATTTTAGCTTAGCTCATTTGTCAAGGTCACCAGTTAT
ATATAATAGAAATCAGCAAGCTATTTTCTATGAATAACCAATGAATGGGAGGGCTTATTGGTTACATAAGACCAATAAATCCTTGTACTTGTAT
TAAATATCCCCCTTTGGTTCCATCCAGTAGTCACACACAGTATTTATTTGTCACGATGGAAAACACACTGTAAAATAGTACATGAGAGCCCTGT
GCCAGTGTTTGCTGCAAATCAAACATGAGAGTTTTAAAAGGGAAGAAAAATAAATGCTTTGAGCCAGGTGGTGGCAGCACATGCCTTTAATCCC
AGCACTTGGGATGCAGACGAGGGTAGATCTCTGTGAGTTTGACGCCAGTCTCGACCACAGAATGAGTTCTAGGCCAGCCAGGGTACCAAAAATA
TGGATAAGGGCCAGATGTGGTGGTATATGCCAGTCATCTTAGCACTGAGGAGGCTGAGACAGGAAGATTACAGTGTGTTTGAGTTCAGCCTCAG
AAATAGTGAGACCCTGTCTCAAAACGAATTACAGTTAAGGATTATAGATTGAGTTATTTCTCTAGGTAAGTACATTCAZAATTTGAGTAAGTTA
AGAAACAATTTTTGCCTTTAATCCCAGCACTTAGGAGGCAGAGGCAGGCAGATTTCTGAGTTCGAGGCCAGCCTGGTCTACAGAGTGAGTTCCA
GGACAGCCAGGGTTATACAGAGAAACCCTGTCTCAAAAAACCACAGAGAGAGAGAGAGAGAGAGAAACAATTTTTTAGAACGCCTTTCTTTTGT
AAATTGGGAACTTTGTATAACCAAATGTATTTTATTTCATAGATTACTATAAGATCAGCCTCTTTGATCCAAGGATGATCTAAGATCTGAACTA
TTGCAATATTTAAAACTTCTTGAGTGGACATCATGTCACAGTGCAAAATTAAACACTTCATCTCAAGTGATAGGTCACAAACTGCAGGTGTAGT
AAAACAAAAACTATATGAAATTACCCTTAGGCTATGTATATGAATTTTGTTTAGATATCAGTCCAGTCCCCAAGATACCTTACTATATATGCAA
ATGTTTCAGAATCCTAAATCTAAAGCACTTTGGTTCTAAGCAAGAGATTTCCAACCTCCATATATATATATACTTTAGTAATAAGAAATTCCCT
AATATAATCAGTGACATGCAAGATAAAAGTTTGTTTGTTTTTTAAGACAGAGTTTCTTGGTATAGCCTTGGCTATCCAGAAACTCATTGTAGAC
CAGGCTGCCTCAAACTCAGAGATCTACCTACCTTTGCCTCTGGAGTGCTGGAATTAAAGGTGTGTGCTGCCACCACCACCTGACAGATAAAATA
ATTTTTTATAATATACCTTATTTACTCACACACTGTAATACTTCCCCATCTCCCCATTTAGTTCTTGGATGAAATTTCATCTACGGAAACTAAA
GAGTCCAGTGGTACCAGTGAGCCTGTGCACCTGAAAGAAGGGTAATTAACTAAATGAGACATGAAAGTTATAACTCACTGTAGACAGTAAGTAC
AAAGAACATGGCTTTGAGAAGGAGGACACCTGAGTTTTCATTCTGTTCCACAGCTTCCTAACCGGCTTCACCCAGGTAAATCACCCTCACGGCC
CTGTCTGCTCCGTCTTAGGACAGCACACGCTGACTACACAGTCCCAGGCTGTTCTGTAGTTAGGAAATGGCTCTGTTTCCTTACCGAGGAGTGG
TTGTTACTAGGAATAATGAGATGGAAATAACTTTCCTGGCCCTTTTTATTCTGTATAGCACAATTCTAAAACGACATTACTTAGAAGAGGAGAG
GTGCCTCACTCATGGTTAAGAGAGGTAGTGGCTCCTGTAGAGGACCTGGGTTCAATTCCCAGCACCCATAGAGCAGCTCACAACTGTTTGTAAC
TCCAGTTCCAAGGGATCTGATGCCCTCTTCAGATTTCCACAGGCAATGTACACTGTGCATAGATGTCATGCAGGCTACGCCATTCCTAAAAGGA
GAACTAAGACAGCCCTGCATTGATAGAAGGACGTCCTGAGTCATCCCCAGACGTCACTCCTGTGCACTAGTAGCCGTGCATGGCCGCGATGCGA
CCTAACAGTCAGGGTCAGCTTCGTTTCAGGGCAGTGGTTTCCACCTCTGAGGCTTAAATGTTTGCTTCTGTCTGTTATGTTCGCAGTGAGATGT
ATAAGAGAGCTCAAGGAAGAACCCGTATTGGAAAGAAGAACTTCAAGAAACGATGGTTCTGCTTGACGAGCAGAGAGCTCACCTACCACAGACA
GCAAGGTAGAGTTGTGCTTGGGCTTCGTTGTGGCTTTACTCTGCTGGTTCATGGTGCCGCGGTGATCGCTGCACAGTAAATGGTTCAGAGTGTC
TGTCCCACAGTGACACACAAAACTGAACTGAAGTGTTTCTTTTATCACATTATGTAGTATATAATAAAACTATAACGTAAAAGCCTCGTGTAAA
CCAGTTTGAGAATAAAACAACTGGACTTGGTAAGTAAGACAGTATAACAATACAAAGCAGAAAGGCACAGACTTAACAGGTTAACTAACAACCA
CAAGCACTCAGCTAATCTGTTAACTCATGGTGCTCAGTAGGTTTTGAAGTCCCATGAAATCTCATGGGTAAATACCAACTAGGAGAGCTTCTCC
TATGAATACCGAAATGATGACATAAAGGCCTGATACAAGGTACTTTGAGAGAATATAGAGTGCTTCATTGGGAATTCCTGAATAGTAGAATAAT
TGAGAAGAGAGATATTTTAAAGAGATGAGGTCTTGACCCAGAACTCTGGGCTTAAGCAGTCTCTTGCATCAGCCTCCCAAATAGCAGGGACTAC
TGGCATGTGTGCACATCTGCACACACACATATGAGCAGTTTAACCTGAAGGAATGAATAGACATGTATATTCCAAGTATATTAATAAGAAGTTT
TTTACAGTGTGTTGCATTTTAGGCATCTGCATAGTTTGTTACAGTATATAATATGTATACAGTATATAAATATGTAACTAACGATCAGTAGGCT
TACCTTTGTTTTTAAAGCTGTCTTTGAATATTTCAGAATTCTAGTTTATATTAAATTTTATATAAATATTCTTACCTATGGATACCATAGTTGT
CTGATAATGGTTATTTTAATAGCACAAGCACAATTTAAACCAGGCATGGGGGTACATACCTCTAATCCAGGCTCTAAACAGTGGTGGGAGTTAT
AGGACAGAGACAGACAGATCTCCATGAGGTCGAAGCCAACCTGGTCTACATTGTGAGTCCTTAAACAGCCCAGATAACAGAGAGAGACTGCTGC
TTAAAAAAACAATCAAACAAAAAACCCTAATAATTATAACAACAATAGTGCAATTAAATAAAATTTAAGATAAATTAGTCATCTTCTGTGAATA
AACAAGTTATTTGTTCCTCAGTTAAAACACTATCTAGCCAGGCGGTGGTGGTGCACGCCTTTAATCCCAGCACTTGGGAGGCAGAGGCAGGCGG
ATTTCTTGAGTTCGAGGCTAGCCTGGTCTACAAAGTGAGTTTCAGGACAGGGCTACACAAAGAAACCCTGTCTTGAAAAGCCAAAAAAAAAAAA
AAAAAAAACACCAAAACAAAACACTATCTAATCTAACCCAGCATTCCAAAGCCTGCTAGTTAGAAAGTTGTTGAAGTCAGATGATAGTCATGGT
GAATTTCCTGACAATCTTACTATGTACATATACACATCGAGTTTGCATAGGAATTTCATGGACCTCAGAAGCCTCAAGTGTACCTACAGTTGTC
ACATTTAGCCTTTTGTTCTAGCCAGTGATGCTAAAAAAGGATGGACTTTCACAGAATTAAGCTAGTAGAACTGTAAGAATGGATGTATTAGATC
TGTGTTGGTTTTATGTTGGAGGAATACACAGGATGGTGAGATTATTTGCCAAAGCTTACAGTTCTAAGCATGAATAATTGTTGTTTTTAACTAT
TTTGATGGATTTTTTTCCAAATTGGATTACATCCTTTCCTGCTGTCTAAAAAAGAATTCATCGAACATAAGTTCACATTTTCTGATTTCTTGGG
GTGGTCATGGGTGATATTACTGCATTTTTAGAGTGCCAAAGGGTAGGTGCAGCTGTTTTTTCCAGCTGACAGCCTTTGTTCCCAGTTAAACACC
TCAGAAGCTGCGCTTTTGTGTCCGTGTGTTTGCTTTGTACAGTTTATCTCTCACCTCTTTTAGTCAGGCTGTTATCTTAAGTTTCCTTTGCTGT
CAGTGACGTGTCTCACTCCCATCCCTGTAGCCAGCCTTCATCCCTTATGCCTAATATACTAGGGATTAAGGTGCAGATACTATAATTGCTAAAC
TACACCTAAGATCTAAGGCTTCGAAAGCAGATGTAAGGGCTTTCCCTCATAAATAAAGTCTCCGAGCTTTGTATTTCCTGCTTGCTCAGCTTAC
TTCTATCTTCTGGCTGTTTTCTTGATAGCCTAAACTAGTCATTATTTTTTGTTGCTACAGCTACTATATTAAATTTTGCTATACAATAAATTTT
GAGGTGCCGGGCGTGGTGGCGCACGCCTTTAATCCCAGTGCTTGCGACGCAGAGGCAGGCGGATTTCTGAGTTCGAGGCCAGCCTGGTCTACAA
AGTGAGTTCCAGGACAGCCAGGGCTACACAGAGAAACCCTGTCTCAAAAAAAAAAAAATTTTTTTTTACATTTGGTTTGGATTTTCTATATTGA
TACAGATTAACGCTATATTTCGTTACAACATACTATGAATATATGTTTCAACTCTTGTTTAAGGTGTTGTATCTATGCAATGCTTCAAAATGCA
ATATTAAGTTCCATATAATGATCCTTAATTTTCCTCAGAACTTTAAAGTTATAATACTCTGACCTATGGCCATAGTATCATAGTTCCATTCTTA
GCTTACCTCAGAAAATAAGTAGACCCCTAACCTAGTTAGTAGTTACATGGAAAACTACTTGGGAATAACACGTGCTGTAGGTGTGTGTGTGCGC
ATATTTTATTTTTATATATATTGCTCCATTGTTAATAGCTTCCATTGTTGCTAGTGAAAATTTTTATATCTGATTATTAAATTTCTGATGTGTC
TCTCTGGAAACTCTTGTAACATTTCTTTAGTAAGTTTTGCTATTTTGTGACCCATAATTTAGAAATTTTTCCTTTTTCTTTTTCTTCTATTCCT
GCCTAACACTTGGTATATCTTTCCATTTAGAGACTCCTTATCTTTTTCTTAAGGGCATTTAGTCACTTGCCAACACTTTTAAGTCCTATGAGTC
TAAGCTTGTATTATTGTTTCTATGTCATTCTGTGTTGTAGTTTTGGAAACTTCTTGGAGAGATCTTTTGTTTCTAACTCACATTGTAATGTTAT
CATGGTTGACAGAAGTACCCTTCATAGTGTAAGAACAGATAGGTAGCCCCTAGAGCATCTAACATTTGTAGTATTAGAATTGGTTCCAAATGAA
TAAGTACAACTTTAGAGTATGGACATGTTGTAAATATCTAACACATTTAAAGAAAGAATATTTTTAAATTATCTGGCCTCCACTGTCCTGTATA
GCTTGGTACCCAACGCTGTCTCTCTGTCTCCTTTCTCCCCCAGATCATACCTCTGATTCATTCCCTCAGATGTTCTCTCTATGTGTAGCTCTGC
TTTCGAATGCTAATTTTCCTATCTATTTGGTTACCGCCTTGACAGTTTGCTCATCTTGTGTTCTACAGTCCATTTACCTACCTTAGGGTTCAGT
AACATACTCACAATGATCATGTTATCTCATTGCACTGCCCTGCCACCCAGCTACAGCCTTGCTTCACTAGCCTGTCTAGTTTCGCCTGATGGAG
CTCATCTGTTAGTTAACCATCCTGCTATGTCTGAGGCTCCAGCTCCAGCCTGCTGTGCCTGCACCTCTCCTAACTTCTCCCCAGCTGTGGCTTC
CATCAGCATTTCCTTCCGACCCTACCTTCTGTCCTCGCCAATGTTATCTTGTCTGCTTTTCTTTTGTTCCTGTAGATGACTGGTATTTGTTGAA
TGTTATTTCCACAGGGAAGTTCTCTAACTGCCCAATCTAGACTAACTTTCCATTACATTTTGTCACATCACTTCAGTTTATCTTCGTCACAGTG
TCCATCTCTAGTTAATATTGCTCTACCAGTCTTTTCAGAACTCAATACAGATTTCACTAGGAACCTATTCCCTGTTTATTGTTCTGTCTTAAAT
TATTACAATAGTACCTGGCATATAGTAGACATTCAAGATGTATCTTTTTAAAAGTACATGCCATGTCTGTTTGAATCACCTTAAATTTTAGGCT
TTGTACCAGGAAAGTGTCTGAACAACTATTTTGTATTAATGTCTTTATTTACAGCAAAAACAAAATCACAATTAACCAGATACTGTTCTTTGTC
AAAAAAAAAAAAAAGGTCACAGCATTAATCCTTTGAATAGTGTAAAATAGAATGTTTTTAGCTTCTTATATTTCAACCCACACGTCCTTAAAGA
AAGGCTTGAGGAAGAAGTAGAAACAAACCTTTGTTGGTGTAAAAATGCCATCACAAAATGACAGAAGTCAAAACTGCATTTGTCATTAGCTTCT
TCTATAACTGGATGAGGGACAGATACCCAGGCTGTACTGACATGCATCATCTTACAGGAGGCCCTTTAACAAACTCAAATTCCCTTACCTACGT
TTCACATTGTAAACCGTTAGGGATTCCATCTGCTCCATAAACTCTCACATTCTCTCTAGCACCTCCTAAGCAAACTTTGTAGTGTCTAGCTAGT
GACTCGCTAACCGGTGACAGCTGTAAATAGTTTAAACCATGCTTTGGAGCTAGCAACATGAGTCACAGGGTACACGATTGACTGTCACCAAGAT
TGATAAGCAGTCCACTGTCTGGGACCCATATGGTAGAAGAAGAGAACTCATTTCTGCCAGTTGTCCTATACCTCTGTACATGTGCTGGGGTACA
CGTGCAGGTACATACATGTGTGCGTTCGCACACACGACACACACACACACATGCACACATATACACACATACACTCAAACACATACACACACAC
TAAAATAAAATGTAATTTTAAAAAAATTTAAACAAGGCTCATGGTGCTCATGGTGGCACACGCTTTTAATCCCAGCACTTACAACGCAGAGGCA
GGTGGATCTCTAAATATGAGGCCAGATTGGTATACAGGATGCGTTCCAGGACAGCCAAAAGAGACTTTGTCTGGAAGGAAGGAAGGAGAGAGAG
AGGGGAGAGGGACAAAAGAGAGAAACTTAAGAATTCACTGCTTTTAACCACTTGGTTTTGCTCAGTTTCCAGCACCAACATGGCGGCTAGCAGT
CATCTGTAACATCAGTCTCAAGATATGGAAAGCCCTCTTCTGACTTTGGCGGGCGGGGCAGAGGGAATTGTTTGTTTTATTTTTTTGAGGGCAC
TGGGCACACATGTGGTACACGTATATACATGCTGCAAAACACATCCACATGCATTACGAATAGAGATGTAGATCACAGCTAGAGCACTTGCCTA
ACATGCATCATGCTTTGGGTTTGGTTCCTTGCACATATACACACACTAAACGATGATGTTGTTGCTGTTGCTGTTGTTGTTGTTTTGAGGGCTA
GTCCTTTAATTAAATGTTAGGTTGACAAACAGAAAGGATTCTACTTGAATAGTCATATTCACTGTGGTCATAGCTCCTGAGCCTGGGCCTCAAA
GTGGGTCTCTGTCACTAGACACATTAGTTCTTTCAGCTGCCACATAATTAGTGTTGCAAATTTGTAATAGCATAGAAAATAATTGTCCATATAA
AGATCAAACTTAGATGCTTTTCCTTGAAAATATTTTCATTTATGTGTTAAAAGGCAGGGACATTCCTTTACATTAAACATCAAAATTCTCTTTC
CGGGCTGGAGAGATGGCTCAGCGCGTAAGAGCACTGGCTGCTCTTCCAGAGGTCCTGAGTTCAAATCCCAGCAACCACATGGTGGCTCACAACC
ATCTGTAATGAGATCCGATGCCCTCTTCTGGGGTGTCTGAAGACAACTACAGTGTACTTATAATTAATTAATTAATTAATCTTTTTTAAAAAAA
TTCTCGTTCCATATTATACATAAACCATCCAGAAGTCTATTGTGGATGCATATGCTTCTATCAGCTAGTGGAATTAGCCTTCCAGAATTAACAT
CTCATCTTTATGGTTATTACAGTAATTCTTTTAATTTTGTCGTCTTTTATTATTTAGGCAAAGATGCAATTTACACTATTCCAGTAAAAAATAT
TCTTGCTGTGGAAAAATTGGAAGAAGGCTCTTTCAACAAGAAAAATCTAAGTTGCATAAATATTGAAGCATTTTAAGTCATCAGCTGTACGTAA
TTTTGGTATCTACCATTCAGGTCTCTCAATAATGTCCCAAGTTAGGCCACTCAGCTGGTGCAGGACCCTCCAGTCCCCAGGATGACATCTCTGT
TCCTTAGCATCTCCTCACTAACTTGTGGTTCCCCCAGGTTCTCCTCCATCTGAAATTACCTTTTTCTTCTTATGCACACAGTGGCCTCCTCTGC
ACTTGTATGGCATCTGCAGTCTGTATTGTTCTGCTTAGCCCCATTGGCTAAGATGATCTTTGGTACTGACCTGCTTACTGCACCAATTCATTCC
GACACATTAGCAGACATTTCTTAGCCAAGCAATCCAGGTCTCCTAAGTGCTTTTTCTTTTTGTATAGTATATGTTGTTTTTAAGGTTTCTCTAA
CTTTTTATTTTAATTTGCAATAAAGATGTTCCAAGTGATACATACAGAGAAAACGCTGTACATCCAAGCAAACAACTGTGTGGAAGCCAATGAG
TGGATAGATGTCCTGTGCAGGGTGAGCAGATGCAACCACAACAGGCTCAGCTCTTTCCACCCCTCAGCGTATCTCAACGGAAACTGGCTCTGCT
GCCAGGAGACGAGTGAAAGCACACCAGGCTGCAAGCCGTGCACCGCGTGAGTAACTCATGACTGCATGCGGGCGTGGGTGTGAGAGCATGACAG
ACGCTATGTAGAAGTAGCTGGACAGAGGCCGCACGGTGCTGAAGACATTTCTATAAACCGTGTCTAGAAAGGCTTGGGTTGTTGCCCAGTGGTA
GAGTGTCTCTCTCACCTGACACACACAGGGGCTTTGGTTTAATTCTAGTACCACAAAAATTAAAATACAGAGAGTAAGTAAATTTCTATAGACA
CAACTGTAAGTTAGTCGTGCCCAGGGGCTAGATTGATGCATTGTTCCGTGAACAACTATAGGTTATATATATGTTTATTGGGACTGGGGAGCAG
TTAGTGCTTGTTGTTCTTGTAGAAAGAAGACTCAACTTTAGTTCCAAAACCCATCTCACACCAGCTCCAGAGCACAGATCAGATACCGTCTTCT
AGCCTCTGCAGAGGCATGTATAGGCACGCACGCATGCGCACACACACACACAGATATACATATATACATTAAAAATATTTTTTATGATTTTACT
TCAAAATCTTTTTCAAGACTTGAGTCCTTAACAACCAGACCTTGTGATCCTGTTG
MOUSE SEQUENCE—mRNA
ATGGCGGCGGCAGCGCCTGCTGCGGCGGCGGCTTCTCCCGAGGCGCCGGCCGTCTCGGGCAGCGCAGACCCGGAGACGGGAGACGAGGACAGTC
GTGAGGTCCGAGTGCTGCAGAGCCTGCGCGGCAGGATCTATGAAGCAAAAAATTTATTGCCATATCTTGGACCCAACAAAATGCGCGATTGTTT
CTGCACTATAAATTTCGACCAGGAAGAAGTTTATCGCACCCAGGTTCTTGAAAAGTCTTTAAGTCCATATTTCAGTGAAGAATTTTATTTTGAG
ATTCCAAGAACTTTCCAGTATTTGTCTTTTTATGTTTATGATAAAAATGTTTTACAAAGAGATCTTCGTATAGGAAAAGTAGCCATCAAAAAAG
AAGACTTGTGCAGTCATAGTGGTAAAGAAACTTCGTTTTCACTGCAGCCGATTCACTCAAATTCAGAAGTTCAGGGAAAGGTTCACCTTGAGTT
AAGACTGAATCAGCTGATAACACAGAATGGAACCCTGTGCCAGCAGCTTGTTGTACACATCAAGGCCTGCCATGGGTTGCCTCTCATAAACGGA
CAAAGCTGTGATCCTTATGCAACAGTGTCCCTCGTGGGCCCCTCTAGGAATGACCAAAAGAAGACAAAAGTAAAGAAGAAAACAAGCAATCCAC
AGTTCAATGAAGTCTTCTATTTTGAGGTAACCAGATCCAGCAGTTACAGCAGGAAGTCCCAGTTCCAGGTGGAGGAGGAGGACATTGAGAAGCT
GGAGATCAGGATTGACTTGTGGAATAATGAAAACCTAGTCCAAGATCTTTTCCTGGCAGAGATTAAGCTTCCTGTCAATGTGTTAAGAAGTGAT
TCCTTTCATCAAGCCTGGTACTTGCTACAGCCAACAGACAATGGAAACAAATCATCTAAAACCGATGACCTGGGATCTCTTCTGTTAACTCTCT
GCTATACAGAAGACTGCGTGCTTCCATCAGAGTACTATGGACCGCTGAAAACTTTGCTGCTAAAGTCACCAGATCTTCAGCCAGTATCCGCCTC
AGCTGCTTATATTTTGGGTGAAATATGTCAAGATCAAAAGGATGCTGTTTTGCCCCTTGTACGACTGTTGCTGCATCACAATAAACTTGTCCCT
TTTATCACTGCGGTGGCCGAGCTGGACTTGAAGGATACTCCAGATGCCAACGCAATATTTAGAGCAAACTCCCTGCCTACCCAGTCTCTGACTG
AGATGATGAAGATAGTGGGAGGCCACTACCTGAAAGTCACGCTGAAGCCCGTTCTAGATGAGATATGTGAATCCTCGAAATCCTGTGAAATAGA
TCCTGTTAAGTTGAAAGAGGGAGATAATGTACAGAATAATAAGGAGAATCTGTACTACTATGTGGACAAAGTATTCAATACAATCGTAGGATCA
AGTGTGAGCTGCCCCACTGTGATGTGTGATATTTTTTATTCTCTCAGGCAAATGCCTGCTAAGAAATTTCCTAATCACCCTCATGTTCAGTACT
CTGCAGTGAGCAGCTTTGTCTTCCTTCGTTTCTTTGCTGTAGCCATACTCTCACCTCATGCTTTTCATTTGCGACCTCATTATCCAGATACACA
GACAGTTAGGACATTGACTCTCATCTCAAAAACCATTCAGATTATTGGAAACTGGGGATGTCAGTCCAGAAAAAAGTCACGGTTCAAAAAGTCA
GTCATGTGTGAATTTCTCAAAATGTTTCAAGAAGAAAGATATTTTACAGATGTGAAAAAGTTCTTGGATGAAATTTCATCTACGGAAACTAAAG
AGTCCAGTGGTACGAGTGAGCCTGTGCACCTGAAAGAAGGTGACATGTATAAGAGAGCTCAAGGAAGAACCCGTATTGGAAAGAAGAACTTCAA
GAAACGATGGTTCTGCTTGACGAGCAGAGAGCTCACCTACCACAGACAGCAAGGCAAAGATGCAATTTACACTATTCCAGTAAAAAATATTCTT
GCTGTGGAAAAATTGGAAGAAGGCTCTTTCAACAAGAAAAATATGTTCCAAGTCATACATACAGAGAAAACGCTGTACATCCAAGCAAACAACT
GTGTGGAAGCCAATGAGTGGATAGATGTCCTGTGCAGGGTGAGCAGATGCAACCACAACAGGCTCAGCTCTTTCCACCCCTCAGCGTATCTCAA
CGGAAACTGGCTCTGCTGCCAGGAGACGAGTGAAAGCACACCAGGCTGCAAGCCGTGCACCGCAGGCATCCCTGCAGACATCCAGATAGATATC
GATGAAGACAGAGAAACAGAAAGAATTTATTCTATTTTTACCCTCAGTTTACTTAAGCTGCAGAAAATGGAAGAGACCTGTGGGTCTATAGCTG
TGTATCAGGGGCCACAGAAAGAACCTGGTTATTCTAAGTTCACCATTGAGGATTCTGTTGCAACCTTTAAGACAATTCAGCAAATCAAAAGCAC
CATAGAGAAGCTCGATGAACCTCATGAGAAATACAGAAAGAAAAGATCGAGTAGTGCAAAATACGGAAGCAAGGAAAATCCAATTGTGGGAAAA
ATATCATAG
MOUSE SEQUENCE—CODING
ATGGCGGCGGCAGCGCCTGCTGCGGCGGCGGCTTCTCCCGAGGCGCCGGCCGTCTCGGGCAGCGCAGACCCGGAGACGGGAGACGAGGACAGTC
GTGAGGTCCGAGTGCTGCAGAGCCTGCGCGGCAGGATCTATGAAGCAAAAAATTTATTGCCATATCTTGGACCCAACAAAATGCGCGATTGTTT
CTGCACTATAAATTTGGACCAGGAAGAAGTTTATCGCACCCAGGTTGTTGAAAAGTCTTTAAGTCCATATTTCAGTGAAGAATTTTATTTTGAG
ATTCCAAGAACTTTCCAGTATTTGTCTTTTTATGTTTATGATAAAAATGTTTTACAAAGAGATCTTCGTATAGGAAAAGTAGCCATCAAAAAAG
AAGACTTGTGCAGTCATAGTGGTAAAGAAACTTGGTTTTCACTGCAGCCGATTGACTCAAATTCAGAAGTTCAGGGAAAGGTTCACCTTGAGTT
AAGACTGAATGAGCTGATAACAGAGAATGGAACCGTGTGCCAGCAGCTTGTTGTACACATCAAGGCCTGCCATGGGTTGCCTCTCATAAACGGA
CAAAGCTGTGATCCTTATGCAACAGTGTCCCTCGTGGGCCCCTCTAGGAATGACCAAAAGAAGACAAAAGTAAAGAAGAAAACAAGCAATCCAC
AGTTCAATGAAGTCTTCTATTTTGAGGTAACCAGATCCAGCAGTTACAGCAGGAAGTCCCAGTTCCAGGTGGAGGAGGAGGACATTGAGAAGCT
GGAGATCAGGATTGACTTGTGGAATAATGAAAACCTAGTCCAAGATGTTTTCCTGGGAGAGATTAAGGTTCCTGTCAATGTGTTAAGAAGTGAT
TCCTTTCATCAAGCCTGGTACTTGCTACAGCCAAGAGACAATGGAAACAAATCATCTAAAACCGATGACCTGGGATCTCTTCTGTTAACTCTCT
GCTATACAGAAGACTGCGTGCTTCCATCAGAGTACTATGGACCGCTGAAAACTTTGCTGCTAAAGTCACCAGATGTTCAGCCAGTATCCGCCTC
AGCTGCTTATATTTTGGGTGAAATATGTCAAGATCAAAAGGATGCTGTTTTGCCCCTTGTACGACTGTTGCTGCATCACAATAAACTTGTCCCT
TTTATCACTGCGGTGGCCGAGCTGGACTTGAAGGATACTCCAGATGCCAACGCAATATTTAGAGGAAACTCCCTGGCTACCCAGTGTCTGACTG
AGATGATGAACATAGTGGGAGGGCACTACCTGAAAGTCACGCTGAAGCCCGTTCTAGATGAGATATGTGAATCCTCGAAATCCTGTGAAATAGA
TCCTGTTAAGTTGAAAGAGGGAGATAATGTAGAGAATAATAAGGAGAATCTGTACTACTATGTGGACAAAGTATTCAATACAATCGTAGGATCA
AGTGTGAGCTGCCCCACTGTGATGTGTGATATTTTTTATTCTCTGAGGCAAATGGCTGCTAAGAAATTTCCTAATCACCCTCATGTTCAGTACT
CTGCAGTGAGCAGCTTTGTCTTCCTTCGTTTCTTTGCTGTAGCGATACTCTCACCTCATGCTTTTCATTTGCGACCTCATTATCCAGATACACA
GACAGTTAGGACATTGACTCTCATCTCAAAAACCATTCAGATTATTGGAAACTGGGGATGTCAGTCCAGAAAAAAGTCACGGTTCAAAAAGTCA
GTCATGTGTGAATTTCTCAAAATGTTTCAAGAAGAAAGATATTTTACAGATGTGAAAAAGTTCTTGGATGAAATTTCATCTACGGAAACTAAAG
AGTCCAGTGGTACGAGTGAGCCTGTGCACCTGAAAGAAGGTGAGATGTATAAGAGAGCTCAAGGAAGAACCCGTATTGGAAAGAAGAACTTCAA
GAAACGATGGTTCTGCTTGACGAGCAGAGAGCTCACCTACCACAGACAGCAAGGCAAAGATGCAATTTACACTATTCCAGTAAAAAATATTCTT
GCTGTGGAAAAATTGGAAGAAGGCTCTTTCAACAAGAAAAATATGTTCCAAGTGATACATACAGAGAAAACGCTGTACATCCAAGCAAACAACT
GTGTGGAAGCCAATGAGTGGATAGATGTCCTGTGCAGGGTGAGCAGATGCAACCACAACAGGCTCAGCTCTTTCCACCCCTCAGCGTATCTCAA
CGGAAACTGGCTCTGCTGCCAGGAGACGAGTGAAACCACACCAGGCTGCAAGCCGTGCACCGCAGCCATCCCTGCAGACATCCAGATAGATATC
GATGAAGACAGACAAACAGAAAGAATTTATTCTATTTTTACCCTCAGTTTACTTAAGCTGCAGAAAATGGAAGAGACCTGTGGGTCTATAGCTG
TGTATCAGGGGCCACAGAAAGAACCTGGTTATTCTAAGTTCACCATTGAGGATTCTGTTGCAACCTTTAAGACAATTCAGCAAATCAAAAGCAC
CATAGAGAAGCTCGATGAACCTCATGAGAAATACAGAAAGAAAAGATCGAGTAGTGCAAAATACGGAAGCAAGGAAAATCCAATTGTGGGAAAA
ATATCATAG
HUMAN SEQUENCE—GENOMIC
CTTCCTTTTCCTGATCCTGCCCAAAGTGAGGAGGTTTGTTAAGCAGATAGGGGAAAGGCTGAGGGCTTCGGAGTACTGCATCTTTAGGACCCTG
GGAGGAGTTCACCTTATCGGTTGCCTGACACCACCCCAGAAGTGGCTATATCAAAGTTTAACTCAATAGCACAGCATAGCTTGCAGGGAAAGGG
GTGAAATATAATTTCCTGAGCTTCCCCCAAGACCAGTACGGACTAGACTGGACCAAGGAGTTTCCACATACTCCAGAGAAGAGGCGTGGAAAGC
AGCTGAGGTGGTAGCCACCAGGCTTCCAGGCACTGGCCATCACAAGGGAAAGGTGAATCAGCAGCGAGGCTGGGGAGAGATATTCTAGTGAAGA
GCCAGATGGAGGATGTAGGGTCTGAGAGGCAGCCAGAGTGGAGTCTGAGTCATCAGGGAAAACAAGCAGGCGCTGCCAAGCCAGGAGGAACCAA
ATCACAGGTACTATAGATACAGCCTCAATAGCAGGCCCTCACACAACTCCCAGGATTGAGCCCGTCTCACCCTCAAAAGAAGTATCCAGAAAGT
ATCCAGACCACCCACCCACAACAGAAATCTGCAAAGATGCAAAAGATACTGCATTGATACAAGGACCCCACCTCTTCTCTGTCTCTATGGGGTC
ATTTAGCCACAACTCCATCAAAAACTCAGACACCATCTCAAAAAAATATAAAAAAAGAACGCAGATATCTAAGAGGCTGAGCATTATTTACCTA
AATGGATGGACCATTTATATAATGTCTATTTAGGTCGCTGACACCTGTAATTCCAGTACTTTGGGAGGTCAAGGCAGAAGGATTGCTTAAGCCC
AGGAATTTGAGACCTGCTTGGGCAACAAAGCAAGACCCCCATCTCAACAAAAAATAAAAATAAAAATAAATGGCTTATACCTATAGTCCCAGCT
ACTCAGGAGGCCAAGATGGGAGGATCCCTTGAGCCCAGGAATTCGAGGTTGCAGTGAGCTATGATCATCCACTGCACTCCAGCCTGGGTGACAG
AGTGAGACCGTGTCTCAAAACAAACAAACAAACAAACTGGTGCATCAGAATATTTTCCCCTCTATTCAGTTATTGGAGACTCATAAGGAAGAGA
AGAACAATTTTAGAGAAATGAAGATGCTACATCTTCTTTGCACATCTGTGGGTAGGATGAAAAAAATCTGCAACCCTTCACACATACTAGTGTT
GCCAGTTTTTCTTGTCCTCCTCCCCACTGCTAAATTTGGTGCTGCTTGGGCTGCTCAGTTCCTCCCATCCACCCACACCCAACTGTAGAGAAGA
GCGTGGAGGGTCCACACCGGATCTTTGGGACTTTTGATGCAAAATTTAAAAAAATAAGAGAGGAATAGAAATGACATAAGCCACATGGGTACAT
TCTAGGGAGAAAAGAATCAAAGCCAAGCTTTTCTTGCAAGAAGCAACTGAGAGTGGGGGCCATTGATGTGCTCAGGGGCATCAAGTGGGAGCAG
GAGGACAGTCAGAGATAGAAAGAGAGAACGGGTGGGTGGGAGAGTGTGCTACTCTGGGGAAAGAGGGTGAGGGATCTTGGTTTTGTTGGATATT
GGAGAAGATAAGATGAAAAACATTTTGATAAGAACTGCTCCAGCCTGGTGCTGTGACACACACCTATGATGCCAGCTACCTGGGAGGCTGAAGC
AAGAGAATCCCATAAACGGGAGTTCGAGACTCACCTAGGCAACATAGCGAAACCCCATCTGAAAAAGAAGAAGAAAGAAGAAGAAGAAGAAGAA
GAAGAAGAAGAAGAAGAAGAAGAAGAAGAGGAAGAAGAAGAGGAAGAAGAAGAGGAAGAAGAAGAGGAAGAAGAAGAAGAAGGAGAAGAAGAAG
AAGAAGAAGAAGAAGAAGAAGAAGAAGAAGGGGAGGAGGAGGAGGAAGAGGAAGAGGAAGAAGAAGAAGAAGAAGATGAGGAGGAGGAGGAGGA
GGAAGAGGAAGAGGAAGAAGAAGAAGAAGAACTGCTCTAGATTATATACTTGTATCTCATTTTTTAACAGTAGAATTACTATCAGGACTTTGCT
TCTTTGAACTCTACCCACTCAGGCACTGAACAACGCAGACCTCAGCTTTGACAGGTTTACAAACACAATGACCCTGTTAATACAAAAGATGTAT
TAACATACAGCAGAGATTCTCAACGGGGCGGTGATTTATCTCCCAGAGGATATTTGGCAGTATCTGTAGACATTTTTGATAGTCACAACTGGAA
TATGCTGGTGGCATCTAGTGGATAGAGTCTAGGGTGCTGCTAAATGCCCTATAATGCACAGGACATTCCCCAACGAGAAAGAATTACCCATCCT
AGCCAAAATGTCAGTAGTGCCACAGTTGGGAAACCCTGAAATAGAGAAATGGAGGAAGGGCAGTTGTTTCCTTACAGCTTCCCTTTCCAATGAC
AAAATAAAAGACAGCTTTGTTTCATTCGAAACACGTTCTCAAGTGTGTGATATTCATTCTTAATCAGCACTACATACATATAATAATCTCAGCC
AAACCATATAAATCAGTTCCCTAAAACCCTTTAAGGTAAAAGAATTCCCATTGAAGAATTCCAGATCCTGTGGAAAAAACGGAGTTGAATAAAG
AAGGCTCCAGAAAATTTTGGAAAATCTTTATAAATATTTGTATTTTCAATATGAAATATGAAAATATAATAAAACTAGCACAGCAAATAAAATA
GCTATGACATGTAAGACATGCTTATTATGGGTAATCAGGTTACTCTCTATGCTTTGCTCATTCTGAGAGAATCCTCAGAATCTAGCTTATATTC
CATCACGCCCCTTGAGAAAGTTTAGCTGAGGTGTTAGTACGTTTGTTTTGCCTCATTGCAGATAAGACATTCACTAAGAAGGATTATTTGGAGT
TAAGGGTGCTGGGGACAGTGGTATATTTGGCCTTCCATACAGATGCTCTATAAATGCCTCTGGATTTTTATTTCAATCTCCTTGGCCCCTCTCT
CCAACAGCCCTTCCCAGTTCTATCTCTGCACAGACAGCCCAAGAATTCAGTCCTAAGGACCCACCTTACATGGCTTAAAACAACAATACACAAA
CTAGATGTTTTACATTCTGGTGGCCATGTCGAGTAATTATTTGATAGGCCACGAGCTTTCATTTGTGGCTGTATATCCAAGTCAGTTTTCCAAA
ATTCAGTTATATGATTATAAAATAAGTACCAATGTCTTTAGAACTAGACAAAATACAATTTATTAAAATATTTTATCTTTATATATGTGATAAG
GATTGAGGGGTTCACTCCCTCATTCCATCCCCAATCTTGCCCTCTTTTAGATGAAAGGCAGGAGGCAGTTGCCTAGGAGAGGGCAACCAATAGC
CCTGGCCTAGGCAGGTCAATGCTGGCCTCTCTCCTTCCCTTGGACCCTCAACTGTGAGGGCTAGGATACCAGGCCGGGCTGTTCCTAAAGACTC
TGAGCCCAAGCAGTGCAGCTGGGCCTAGAAATGGGGAGGTAACCCTTCTTGCCAACTTTGCTGAAAATGTGGAAGCCCCGCGGGTTCTGAAGGA
TAACGCTCTCGGCTTGGGCCTTGATAGTCTCATAGCAGGCATCATAAAGTGGTGAGGGTCCCTTTTTGTGACAGTGGCAGGCGGAGCCAGAGCA
GGAGAGGAAGAATATAACAAATCCAGCAACCCCATCATGTTCCCCACACCCTACTCTGCCAATGTAACCCACTGGGCACTCCTGAAAATCAATA
ATTGGGAAGAGGTTTAATTATTTGGGCAGGTAGAAAGTGGAGACACAATAGAGAACTGCGGATTATTTATGTTAACAGGATTTCATTTGTAGCC
ACAGACTGACATGCTGCCTAGATATTTGGTCATAATAACAAGTATGAAAACACCTTGCAACTTTGAAATCAGTTTTCCCAGCTTCTACAACCAC
CCACAAAAAATTGTATCTAGCCTACGTTTAGAACACAGATTATGATCGATGAGCTTCTGCTCTCCTTGGCTGTCTCTGACAAAAATGAGCTGCT
TGTGACAGACGCAGCCCCAGCCCAGTGTGAGGATTGGAAACCTCAGACCCTGACTCTTTCCCTTTCTCCTTCCTTATCTGACTCCTGAGCTATT
ATTGCCTTTTCTCTGCACCTAAGCTTTCTGCAAAGCCCAGACCCAGCCAGCTGGTTCTGTGGTCTGCTCTGATTTCCCTTGTGGATTTTAAATT
CAGAATTCTAGGCTCATTTGTTAGCAGCTTCTCTGAACACATTATCTCATTTATTTGCTCATTCATTCATTCGTTCATTTATCAAACATTTAAT
AAGTGCCTTCTGTGTTCTTCTATATTTTCTATGCGCTAGGGAGGCAAAGACAAAACATTGTCCAGTCTTTGAGATGGTTTCAGACAGGTAAACA
AACAGGAATAGTAAATGTGTTATGAGCACTACAATGATTATGTGTATTCTTATATACATGGTACATAATGGCCTAAAGGAAAAAGAGGCCAGTT
CTGTGAGACAAAGTCAGGAGGTGAAGATTGGGCATTAAATGTATTGAAATATTTTCATTTGAACTTAGGCACTATTATTGGAAGATAAGTATGT
TGATATGGTGAACAGAGTTGGCTGCCTACTCCATGTCTTACTGCTCCCCTTTCTGTGTCACAAGAAAACACTGGTTTGGTTCAGGCTTCAGGTC
TCAAGTAGCAATGTACTTTGGGAAGGAGGGTTCATCCCCAGACCCAGGGGTATCAATGTTGCTTAGACTAAGAAAATTATATATTCCTTGTGAT
TGTAAATGAGCCTTAAGAAGATACCTCATGGTGGTGATTTACAGGAAAGCTACCTGGAAAAGCGTTCCTCCCTAATATTAGGTTGGTGCAAAAG
TAACTATGATCTTGCCACTGAAAGTAATGAAAAAGACATTTTAGAAAAAAAAAAATACACCTTCCTGCCATTGGATGTTGAGATATTTGGAGGT
ACAGGAGCCATATTGTGACCATCAGGCAAAAGCCAAAAGAATTGCAAAGAAGCTAACCCAATAAAGTCCTGTTTATGAATGAATACAGAGTAGA
AAATGAAACAAGTGAATGGCTGCACAACATTTCCAGTTTTCTTTTTACACTTATTGTTTTGTATGTTTTTATTTGTTTGGTTGGTTTTGAGTTT
TGGGGGTTTTTGTTGTTTTTGTTTTTTGATTTGTTTTTTTAATAGATTTGGGGTCTCACTATGTTGCCCAGGCTCGTCTTGAACTCCTGGGCTC
AAGTGATCCACCCAACTCGGCCTCTGAAAGTTCTGGGATTATAGGCATGAGTCACCATGCCCAGCCTACACCCATTGTTTTGTTTTATCACTAA
AATGACAAGAATGCACTGCAGATGGAATGGATATGGCAAAATAGTTCAAAAGGAGGGGGATTGAGAAATAACTGAAGTCACACTGACCTAAGGG
CATCAGCTGTATTGTGCTCAGATATTCAGGCCTTTAAAGTTCTCTTGCAAGAGGTGACAGAATTTGAAAAAAAAAAAATTCTATTTTTAGAGAC
AGGGTCTCTCTCTGTCACCCAGGCTGGAGTGCACTGGGATGATCATAGCTCACTGCAGCCTCAAATTCCTGAGCTCAAACAATCCTCCCACCTC
AGCCTCCTGATTAGCTAGGAGTACAGGCAGGCACGTGCCACCATGCCCAGCTAATTTATTTATTTATATTTGTAGAGACAGAGTCTCACGGTGT
TGCCCAGGCTGCTCCTGAACTCCTAGCCTCAGGTGATCCTCCTGTCAAAGTTGGTATTACAACCGACTGTTGGATTACAGGCATGAGCCATCAC
ATCTGGCCTTGAAAATATTTGTACCGAAGGAGTTGGAGGTTTTAAGGTAAAAATTTGTCCACATTTTTGTCCACACAGCCTTGTCAATGGCCTT
ATTTGGTCTTCAAATGAAATGTTGAAAGTATGTTTTCTGAACATTTAAGGTCTCTCCTCACCTCCTACCACTCCAATTTTATTTTTTAAAATGC
CAGTTGGATTTAGAGATTTTTTTTTAATAATGTAATTAGTGCATAATGCTTAACATCAAACAGTACTCTGATGGGGCAAATGGGGAAACCAAGT
CATCAATGATCAAAGGAACTTGCTTTCCCACCAGTCCGGCAGAAACAGTTCTGTCTCATGAGTGTCCAGCCCTATGAAAATGATCTACATTTAA
ATATTAAAAAGAAAGCACATTTCTCTTCCTCCTACTCGCCAGAAAAAATGTAATAAATAACTCAAGGCCAGGAGACAACCCCTTACAATGCCAT
GTTATAGGTTGGTGGGTGCTGATTCAAAAATATGCCGTAACAGACAAGTTATGGAATTAGTTTCTACCTGCAGGTTATAGTCTTCTTATTAAGA
GACAATCTAGTTTTCCTACATCATCTGCCATAAAGCAAGCCAATGTATGGGTTGAAATGACTGAATGAGTTTAAAACTGGTGAAGAAATAGGAA
AAGACATAGTAGTGAATGGTTGCTCATCAGAACGAAGAGTGGTAGGGATAGAAGGGATGGCCTTTAAACACATTCATTAATAATTTGAGGAAGG
AAGTGAAGTTCAAGTTTGTGATGATGCAATATTGTTCCAGTTACTAAACATCATGAAGAAAAGAGAGCAACTCTAGATGAATTTAAAACATAAT
GCAAGGGCAATAAGGGGAATCAGATGTAACAGCCTTCTCGTAATTATACTGTATGTCTTGTTAGTCTTTAGAAAACTTTATTTTCGGCCGGGCG
CGGTGGCTCACGCCTGTAATCCCAGCACTTTAGGAGGCTGAGGCTGGTGGATCACGAGGTCAGGAGATCAAGACCACAGTGAAACCACGTCTCT
ACTAAAAACACAAAAAAAATTAGCCGGGCGCAGTGGCGGGCCCCTGTAGTCCCAGCTACTAGGAAGCCTGAGGCAGGAGGATGGCATGAACCCG
GGAGGCGGAGCTTGCAGTGAGCCGAGATCGCGCCACTGCATTCCAGCCTGGGCAACAGAGCAAGACTCCATCTTAAAAAAAAAAAAAAAAAAAG
AAAGAAAGAAAGAAAACTTTATTTTCCAAATATTTTATATTAAGGAAACATTGACATTACCCAAGATCTCTTCAAAGTAGGCAAGTATCAGTAA
AAACAGAATAATTAGAAGTGACTTCGGATGAAAAAGCAATGGAGTTTGGGGATTTTAGTGAAACGGTATAAGTCCGAAATCTGCAAAAGGAAGA
CTGTCTCGAAGAACAAAAAAGTGGAGTGTCCCTTTATGTAAAATTGTTTTCATACCTTTAGTGACCATTTAGTAACAGAGTTATGCACTTGGGG
TTAAGCTAGTGAAAAGGAAGGGAAGTTAGAAGATTTTGACACTAATGGACAAAGATATTGATACTTTGTCCATTGGATACGTTTGTCCATTGGA
TACATTTGTCCATTTGTACATTGATACTTTATCCATGGATACATTTGTCCAAAGGTTCAAATAATTTCTCTGTTTATTGGAGAAAGAAATAGAT
GGATGAGGCATCGCTTGGCCAAGCAGGAGGAAGGCCAGAATCAGGCTGGTGAGAAAAGGGGCTCAGTGTGCCTGCAGATCAGAGGAGTCAGGTA
CGCTGGATCCAGTTCCAACTCTTGGCTGAAGAATTTGAACTTTAGTCATTTCTAGCATTGTTCATTACCTGTAGTGTGCTCTTGCTAGCTGGCT
GAAGGGATAAATGAAGCCACTGATGTCCTCTTGAGCAGGCAACTGATAAGCTCCATTTTTTTTGTTAGGAAAGTTAATTTAAGTGGTATGTAGG
AGGAATGAGAGGGATGGGGGACTTATTACCAGAGTTCCAGTCAAAAGCATGAGGTCAGTTACTGAGGTAATCCTACTACGTGCTCAGCATTCCA
CTAGACACTTAACACCCTGCTCCCAATACTTATTTAATCTTAATAACAACCCTTCAAAGAAGAAAGTTTAGCATTTCCACTTTACTGTTAAAGA
AACAGGTTCAACCGGGCGCAGTGGCTTACACCCAGCACTTTGGGAGGCTGACACGGATGGATCACTTGAGCCCAGAAGTTCAAGACCAGCCTGG
GCAACATGGCAAAACCCCATCTCTACCAAAAATACAAAAATTAACTGGACATGGTGGCGCCCACCTGTAGTCCCAGCTACTTAGGAGGCTGACA
TGGGAGGATAGCTTGAGCCCAGGAAGTTGAAGCTGCAGTGAGCTGAGATCGTGCCACTGCACTCCAGCCTAGGCAACAGAGCAAGACTGTCAAA
AAAAAAAAAAAAAAAAAAAAGAAAGAAAGAAAGAGAAGAAACAGAAACAGGTTTAGGAAGGTTGAGTAACTTGCCCAAATGTAAAAGGGGTAGA
AACAGGCTCTATGTCTGATTCTAAAAAGGGATGCCTTTAAAAATAAGGGGGAAAAAACCCTGTAACACGTTCATACTCTTTAAATGAGAATAAT
TTTAACAAGCGCAATGACAGAGAATGGAAACGAAGGAAGAGATTTCAGCAGAGTCAGAAGGATGTGGCAACTGAAAACAGCATTGGGACAAGGG
ATAAGAAAAAGTCAAAGTTTCTGGCCTGAGTAACAGTATAAGATGTGAACTCAGATTTGGAAAGGGAGACAGGAAATTAAGTTTTGGACTTATG
TTTGAAATGACAGCAAAACATTCATATAAGAGGAAGTGGTTCACTTTATCCAACAAGTGTAAAGATCACATGGGGAAATAGAAGTCACCTGCAA
CACAGGCAGAACTGGAAGCTTCGCTGGCCAGGTCCATGTTAAACTCTGACCATTCTCTGGGGCTGTCCAGCCTTTGCACAGGCGGCGCGTTTGT
CTTCAGTTTTCAGGTTTCCCCGTCCTATTTCTTATCTCCCAGTGCCTCTCGCCTTTTCTGTGTCTCCCTCCAGAATGAGCTGGAGCTCTCTTCA
TCTTCACCTTTGTGTAGTCTCTGAGGTCTGAGGCCATCACTATGTCTGACCCTCAAAAAATATTTGTTGACTGACTGGCTTATTCCTGTTGTTG
CCCTGGAAAAGAATTACCCTGCCCGATTAAGTCACAGTTTCTTCCTTCTTAGGGCAGCGTAGTTAACAACCTCAACACCAGGAAATTGTCTTCC
TCATCACATACCAGGGTATTATTCATGCAGTTTACTCACTGTAAAGAGTTCCCTTGAAATAAAGCTAACAATTAGTCAAAGATTTACACATTTC
CTAGGGTCAGTCAACTTTTTAACAAAGCCAACTTTTAGTAGTGGTGGCGGTGGCGGGGGGGAGCCCTTCTTTGCTTTTTCTGTTCCTGAAATAC
AAAAGTGATGGTAAATGCTTCAGAATTTCTCTATGAGAGGGTCTCAGGCACTGCACTCACGCTGGAGTTGGGGGTGGCTGGTCCTTTTCTTGAA
CCAGGTTTTGCAGAGAATTGGTCCTAGTAAACAGTATTTGTGTGGGAGGAGTTTGCCTTGCTGGCGGGGAGTATTTGGATAATACTGGAAGAGC
CTCAAATGCATCTCTTGCCTCCTTTTGAATAAGATACTTTTGAGGTCCACCCACCTGCCAAATTCCATTCTATTTTCTTCCCACAGGGAGTTTC
CCTGATTGGGAAGGGCCTGTATTTCCCGCGAGCGTTAGGAGATGTGCAGGTCTATTCATTGGGGGAGCCCAATTCCCGGGAGGATTTAGGGATC
ACGGGAGGGCCGGGGCGTTCTCCTCGTGGGGATTTTAAGGCCGCTCAGAGCCACGACTGGGAGCTACCCTTAGCCAGCGCCCCAAGCCCGGAGT
ACGGTTCTCTGCAGGGTAGCCCGGCTCGGGGGTGGCGAAGGCGGTGAATGGAGACCCGGGGGGGGCGGGGGCGGGGCCGCGGCGGGCGGGGCGG
GGCGGGGCGAGTCGGGCCCAGGCCCGGGGGCGGGGCCAGGCGCGCGGCTGGCCTGCGCCTAGGCCGCTGCTGGGCTCCGCCTCGCCCGGCTACG
CAGGCGGCAGGGCTGCGGCACGCGCCGGGCGGCACCATGGCGGCGGCGGCGCCTGCTGCTGCGGCGGCTTCTTCCGAGGCGCCAGCGGCGAGTG
CGACTGCAGAGCCCGAGGCCGGGGACCAGGACAGTCGCGAGGTTCGAGTGTTGCAGAGCCTGCGGGGCAAGATCTGTAAGCGGGGGCTGGGCTG
AGGGGACGCCCTGGCCGCGCTGGGCGCGAGGCTGAGGGGGCGGCGGGTTCGCGGCGGTGGCGGCGCCGAGCTGCGCTGGGATCGCGGGCCCGGG
AGGGGGCCTGGGCCGGGCGCGGTTGAGGCTGGAAGGGGGTGTGTTGCGGGGCGGCGTCGCCTCGACGGGGCGGCGTGGGCCGGGGCTGCTGCTC
TCCGGAGGGAGTGGGAGCTGGAGATGGGCCTGGGGTGGGAGAGCCCGGGCTGCGCCGGCGTGAGGGGCTCGGTCGTGGGGAGCGGCCTGGACGC
GGGGACGCTGGAGTCCGGGGCGCGGCCGGGGGCGCGGTCCGGGGGCCTGAGGAGCTTCCGGCCGCCCTCAGACTCAGGCCTTCGCGCGGCTGCG
GGCGCTGGGGAGTGGCCCCGCGGTGGCAGCCGAGCAGTAATTATACGGAGGCGCGGCCCGAGGAGGTGGCGGGCTGGGAAGTGGGGCTGGCCCG
AGGGGAGGCGGCTGTTCGTTTCCTGGGCTCGGCGAGCCTGGGAGAGGTTGAAAAAGGCGCCCTGAGCTGCAGGAAACGGGCCGCCCTGGGGCCC
GAGTGGCTACCGCGCGGTCTGTTCGCGGGTGAGCGCCCCGGGAGGAGCTCGGCGGGACACCTGTACTTGCCCCACCTCGCTGCGGCCGGGACTT
GAATCGAGCCACGGCGTGACAGGCGGGACTAAAGGCAGGATGTAGTGGCTTCCTGTGAGCAGGTCTCAAAACACAATCACCCAGCCCGCACTCA
GCCCGCTTAATTCCCTTCCTTCTCATGCGTGTAATTGCTACTTTAGTGAGCGACCCTACCAGTTGACTAAATACGAAATTTTGCTCAGAGTTGT
CCGAGCATTACTGTGGACAGATTATTTGAGTTCTGTCATTTGAACATTTTAGTTTTAAAAACGACCTTTTGGTGTCAGGTTGCCATTTTTTTTT
TCACAGTGGTTTATTTAGATGAAGTGCTTTTTCTTTTCTGTTTGTAAGAGAGATCAATAACATTTATTTTTGCATTTGTCGAAAATTAGCATTA
TTTTCTTCATGCAGTATTCTCAGATTGGAAACATGCTTCATGTTTCTTATAAATAACCCTCAATTATGAGGGCGTACTTTTCACTTTGAAGAAA
ATTGACTTGCATTAAAGTGCCTAACAATTCTTTCCTGGGCACGATGTAAAATTTTCCTCTCCTCTAATACCAGTACTGTTGAGCTCACATTCTC
CCACTTTTCCTCTTTTCAGGTGGTTCACGTATTTGCGATTTTATGAAACCTCACAACCAGACATGTTAACTTTTCTTATCTTTTTATTCCCTGA
GGTAGTCCTCGGGCTCTTAAGAGATTACAGTTCTTAAAACCTGGAAAGTGACACCAGAGAGGTAGATCTTAGTTCCCAAAATTAAAGTTACTTT
CTAGGGCATAAAACCTTTTCAGAATTCAGATTAAATTTTATTTATTTTTTCTTTTTTCTGTAACCTTATATTTGAGGGGAAAATTTTATTTTCA
ACTTTTGCATATATCTAATTTAACATTTGGGAAAACTGTAAATGGGCCAAAGTTTCTCCCTTTATATGATTTTCCAGATTTTTACCACTTTCTT
AGTGCCACTTGATGCTAGGCATTGTCTATTGGAGACTCACTGGTACGTAACTGCAGGTTTTACCATGGAACTACATATACACATGTCTTGGAAT
TGAGGGTTAGGGTTTCCAGAAGGACTTAGTTGTCCTGTGCTTTTGTCTGCCCCATGCCAAAGACCACTAAGAACAGTTTTGTAAGTGAAACTTG
GGTCTACACGTTAAAAAAAAGAAAAACAAAAAACCACTTTGTTTCTATGGTTTAAAGCAACCCTGGCTTGGAAATTGATCATCCAGCTGCTGGG
AATGATATTACTGCTTTAACCACAGTTGAAGTATTCTATCTGTTATCCTAGTCAGTTTCTTGCTTTTTAAATTGCTTATCTTATAGCTCAGGTT
ATGGAGTGTCTGCTTTCTAGAAAAAAAAAATGTATTTGTATACTTATATACAGAAGCTCTCTTCCCATTTCTTTGTCCTCCTTTTAAATGATGT
AATCCACAGTTGGTTGACATTCAGAACCTTGTGAAGAGATGCTGTTTTCTGTTTCCCTGAAACTAACATGTGGGTTTTGTAGCGTGGGGACTGA
GGGGTTGGAAGAGTCATTAGCAGGTGTCAAGGTCACAGAAATGAGATGGTGGTGGTTCTGACAAGATATTAAGCAGCTTCGGGAGGGTGTAACC
GTGTCAGCTTTTCTCTTCACCACAGTAGGAAACCTTGGAAGAAAGCCTGATAGTGCCCACTAGGAGATGTGCTGGCAGATTTTATACTACTGAT
TGAGAGCCTCTTTTCTGGAGAGTTAGAGATCTGTAGGGGCTGAAAATTAGCCTCAAGAAATTTTCTTATTTCTGGACTTCCATAGGACTTTTAA
ACTGCACATCACCCCACTGAAAAGTGAGGATGGATTGGTTTATGTATCTTGAATGTTCAAGAGATAATAGTCCATAACCTTCTTTAGGGATTTC
ATTCCTGTAATTATTTCCCCTTATACTTCACAAGGTGAAATATCTGTCAGTTGGACAGATATTTGTCTCTTGGACAAGAGGAAACTTAACAAAA
AGTGGTAACCAAAAAGGTGCAGCTCACAAAATAACAACATGAGACCTCAATATATATAAAGGATGAAGCCCCAATACCCCTTTTTTGCTTGAGT
GTACTTTTTTTTTTTTTTTTGCCGGGGGGCTGGGGAGGAGGGAAGAAGGGACATGCAAATATGTTTCAGGTGTTTTATATGCGCTCAAACGAAC
CCAGGGAATTCGAGATCCATCTATAAGACAAAATATTGTCAGCAACCCTGCATTTGCTGCAGTTTGCGCTAGGAAACCATGACAGTTCTGAATT
GTCTGCTTCCTTAAACTTTTTCAAGAGGTGAGTTTGGTTGTTTGATATCCTAACGTTGTCATATGAACCCTTGATAAAGGAAATTAGGGGTGTA
CTCGACTCCATTCTTCGGTCTGCACTCCTGTGTAGCGAGTAGCAAGTATGCTAGAACACCTGAAACACATTTGCATGCTCCCTCCTCCCCTCAA
AAAAAAAAAAAAAAAAAAAACCGTATTCAAACAGAAAAGAGGGATTGGGGCTTCCTCCTTTATGTATCTTCAGGCTTCATGCTGTTATTTGGTG
ACCTCCTTTTTGATTAGCACTTGTTAAGTTTCCTCTTATCCAATTACATTAGATTGTAAAAATGTTGCGGAGAGCACCCATGCCACCATATGTG
TGGGCTTATCTACTCAAGGAATACCAGATCAATAAGAGAGCCAAATGGGAAAATTTTGTGTGAATTTTCAGATGTGTGGTACAGTGCGAGATCT
GTGGGACTATGTGTTCCAAAACTCACTTAGTTTCTCTGGTGTCTTCAGACCAAGAATAGCCTAGGGTTATTTGACAGAGTAGGGAAGATCTATA
GGGTATGGTTAAGTGCTTGAGCTTAGAGGCTGGGTGAAAAAGTTAGTTACCTGGGTTTGAATCTTCACCTAATTTCCAACTTTTGTTAATGTCT
TTGGGCCTTCATATTCTCATATATAAAATGGGGATAGTAAAAATACCTATCTCACCGGTTGTTATCAGGTTTAAATAAGCCTCCCTCCCTACTC
TGCCCCTCCTCATCTCTGCCTGCTCTTTCTCACTCCCCAGGATCTGGGTCATACACCGACCATCTCTTAGGAAGGAGTAAAGTAGGAGATAAAA
ACTAATCTTCATCTGATGTTCTTGTCCAACGTATACTGGAGTTTCCAGGTGTACAAAGCTGATTAAAATCTGAAGACCTATATTCTTCTTTAAA
CGTTGTTAATTAATCTTCATATCGCCTTACCCAGCTCACTTATCTTCAACCTTACCTTCCGCATCTTTTAAATCGGAATGATGCTTTTCCACTA
TCCATTGTATACAGGTTGTTCCCTCTGCTTGGGATTGTCTTTCTTCTTTACTCCACCCCATCTTTATGTCAGGAAGCTCCTACTCATCCCTCAA
AAGCCTGCTCTATGTGATCTCCTTCCCAAAACCTTCTGAGACACTTCCAGTGCCATCTCTGGCCAGCATCTCTGCTGCTGGGGAACACAATTCC
TTGCTCATTTCCTTATTATAGCACATTTCATAGAAATCATTTGTTCACTTCTCCTGTTAGATTGTGAGCTCTTTCAAGATAGAATTGATTCTTG
TTCATTTTTAAAAAATCATCCCAGTGTCAGCACAATGCCTAGAACAGAATAGCCCTTTAGAATATGCCCTACCTCACAATGTTATCCTGTGGAT
AATTACTACATATGAAAGCATTTTATAAATTATATACCTAAAAAAACTACAACGTGTTAGTATTAAGAGAAAACTGTATGAGTCTGCATCAGGA
AGTTCATCCCTTTCAAAATCACAGTGCCCACTAAGCTTAGGTTTGAATTGATGCCAAAGTTGCCTTGTTAGGAAAAAGATTAAATTTTTCTTAA
TGCTCTGATTACTTATTATTAGGGCATTTGAATATTGTTACTAATTGGTCCTCGAGCTTGGAAAAGAAAATCTCATATTCATACTTTACACATT
AAGTGTTTTAATTTGACATGACAGGTTTGAATAAAACCGTCTATTTAATTCTGTGAAGTATAAATGAAAACATTTTATTGACTATCCAGTAAAG
TTGAGTGGAAGAGATTAAAAAGGGTGTATTCATGCTAAGAGTTCATTTTTATCAGATATTTGAAATGACATGATGAGTTATTTACGATGGAGGA
AGAATCCATCTTTCAGATCGATAGAAACTGGGGAGAAAACCGTTCTTTCTAAAAGTTCACACAGCAGTCTGCTAATCATTTGCTCTGTGGAAGA
CGGGAGGTAAACCTCAGTGAGTTCATAGAGTTGTTTTTTTAAAACTTAGAACTCTTTGAATAGATCCTTAAGTGACTAATATAGCCAGAATAAA
ACCATCATTTTATCCTTTAATCATAGCTGCTAATCCTATTTCCTTGTTAATGTGCTTTATGAAATTATTTGACAATAATATCAGATTACATGTT
TTTGCTACTAATAAAGTTCTATTCTCTTTTGAGAACTGCAAACAAAATGTTGTATAGTCATATATTCATGTAAGTTAAGCTTTTTATGTGTATT
AAGACATTTTTATGGCATCTCTTTAGGTTTGTATGTAATTTTAGTTATCAAAAACCCCTATCTAAAGTGATAAACATTAAAAGTTTGTATCAGT
ACACATACAGACAACCATAAATGACAAATTTCTTTTCCTGTCATTAATCACAATCACTTTGGCCAAATTCTTTAACCTTGCTGAGCCCGTATTT
CCTTACTTGTAAAATTAGAGGTTTGGATATGGTCTTTAACATCCCTTCTACCATTTTGATTATTGCTTTTTAACTTGAACCATTTGCTTTAAGG
CAGTACATCAGATGCTTTATTTTAGTGTTTAAATGTATTTAGTACACTTGTTTTGCATGATTCACTGTTTTAAAAATATTTAAGATTTATTAGA
TAAGAGCGTTTGAATTCTAAATTTTTTTTCTGTGACTGTTAATTAATTCAGCAGCACGTAATACATATCGAAGGGTCTGTTAAGACACGCTAAC
CCACTGGACTGAAATGGGCCTAAGCTGTGAATCGGATGAAAGATCCTATTTTATCCTAAAGTCTATACTAAGCAATAGCTGTTTTTAGACATCT
GTATTCTTAGACAATTATAGAAATTTAAAGCTGGAAGGTAGAGAACATGAAGGTAGAGAAAATAAAGGTTCTTACCCTTCATTTTACAAATGAA
GAATCTAACAGAGACAGCAAGTGTCACACAGTTGATGGCCTGTTCCGCACTAGTCCACTATTATCAAAGTTCTTTCTAGAATGCTATATGAGAT
TAACTTATTACTATGAATGTTGCAAATGTACTACATTTAATACACAGACATTATACTACTTATCTGTTTATATATTGACAGGTGGTAAGATTTT
CACTGTGCCGTCTACACACTCTTAACTGTTCCTTCTTAAGAACTTTGTGAAGTGGTTAGGATTAGTGATGAAACTATGGTTATGACCTAGGTGG
AGATTGAACCTGGATGTTTAGTTACTTGTGCCATGGTCTACCTCACCTATTTTCAGTTCTCACTCTCTCTCACCATCACCTGTGAACTAAAAAA
ATTATGATGCCCACACTGTACCCCAGACCTACTAAGTCAGAATTTCTGGTTTCAAAAGTTCCATAGGGGGTGATGTTGGGCAGCTAGCATAGTG
AATTTCTGCTTTTGTCACAAATAGATTAGCCACATACCCTACCTAAATGATGGCAACCTGCTGTGCTATCATTTAAAACGTATCAGTAGAAAGG
TTGGTATAGTTACAGTGAGATTTTAGTTTGTTTGCATATATATTTGATGTATTAATTCTATTTGATTAGATAATATGAACACTTGGTACAAAAT
TCAAGAGGTACAAAACAGTATATACATTGAAAAGTATTCCTTCCTTTCATTATCTTCTAGCCATTCCCTCCCAAGAAGCAAGCATTACCAATTT
GTTATATCCTTTTTCTTCTGTCTTCTTCCACAAATTAAGCATTTACAAACATAAATTTATCTGCACTAATATATTCCTTCCACCAACATACAAA
TTATAGCAAAATATATACATTTTTCTGTATCTTGCTTTTTTACACTTAACAACATAGCTTGAAGATTTTTTCACATTCACATAAAGAGAGATCT
GCCTTATTTTTTAAACAACTCCATAATATTTCATCATTTCGCTGTACCACACTTTATTTAGCCAGTCCCCTATTAGTTGACACTTAGGTTGTAA
ACAGTCATTGCTATTACAGACATTACTACAATGAATAACCTTGTAAGCATGTCATTTTTAATATGAGTGTCTTTTTGTAGGATACATTTCTAGA
TAGATGTTAAATTGATGAATTGAAGGATATGTGCATTTTTATTTTGGAAAGACAATGCTAAACTATAGCAATTTAAGCTCTCACCAGCAATGTA
TGACTGCCTATTTCTTTATACCCACGTAGGGTGTATTTTAAAACTTCTTAATCTTTACTGGTCTATGAAATAAAAAGGAACATTTAATTTGTAG
TTTTAATTTGCACTTTTAAATGACAGTGAGGATTGCTCTGTCACCAGGGCACGATCTTGGCTCACTGCAACCTCCGCCTCCCGGGTTCAAGCGA
TTCTCCTGCCTCAGCCTCCCGAGTAGCTGGGATTACAGGCATGTGCCACCACGCCCAGCTAATTTTTGTATTTTTGGTAGAGACGGGGTTTCAC
CATGTTGGCCAGGATGGTCTCGATCTCCTGATCTCGTGATCTGCCTGCTTCAGCCTCCCAAAGTGCTGGGATTACAGCCGTGAACCACTGCGCC
TGGCCCTAATTTTTAAATTTTAAATTTTTCTAAGGCTTGAAATTAAGTTATAGGATCTCTTCATATTTTTAAGAGCCATTTTTTTTGATGTGCT
ATGTGAACTGGTCTATTTCTTTTGTCCTTTTTTTTCTGGTGAGTTACAGTCTTCTTGATTTGTGAGAAAATGCTTTATGCTGTAAGGAAGTTAG
CTATTATTTTAAAACCTGTGCAAAGATTTAAAAAATCTGTATACTTTGGCTGATGCCTGAATTTATTCTTAAGTTGATTGTGACATCCAGTGAG
GGCAGTCAGTTGTAAGTTTCATCTACTTATATAACGTCTGATGGTTGAGAAATTGAGAAAGAACTGCTTGAGGAAAGGTAAGTGAAGACTTATA
AACAAAAGTTAAAAGACAATAGATTGGGAGAAAATATTTCCACTGTATCCTTCAGACAAACGAAATGTGTATAACACTGAATTCTTTGCCAGGC
ACTGTTTTAAAGCACTTTATGCGTATTAACTAATTTAATCCTTACAGTAACTCTAAAAATACCATTATTATCCCCATTTTACACATTATGAGGA
AACAGATACACAATAGCTAAGCTAAGTAAATTGACCAAGGTTAGATGGCTGGTGAATGGTGGAGCTATCAATCAGACTGGCCAGATTGGCTCTA
GAGTCCATCCTGTTAACTCTTAACCTACTCTGCTATTATGCCTCTCTATTTATTATCTCCAGATTATATAAATAATTCATACAGACTTAAAAAC
AAGAGGCCATTAGAAAAATAAAGGATACGAATAGGCATCTCACACGAAAGGAAATACAAATGTCCAGTAAACATGAAAATATTGGTAAAGTAAA
AATTAATCAGGGAAATAAAATTTTAAAACATCAATTTACAAAAAAAGGAAAGTTTATCTCAGTCTTTGGTGAGGGTTTAGGGAAATAAGTATTT
TCCTCATTGTTGTTGTGAGTTTAAATTGATAAAGCAATGTTGGAGAGCATTTTGGCTATATCTGTTAAGAATTTAAATGTCTATACACCATGCA
TTTTTACTTCTACCTATTTGCCCTGCATCAACAGTTGTACATTTAGAGAAGAGGCATATACAAAGATATTCAATGTTGCCTTGTTGGTAAAACA
GAATAATTGGAGACGACTTAAATGTACATCGGTAGGAAAATGGATAATATGTTCATACACGTATTCTATAGCAACTAAAAAGAATGAGGTATAT
CTATGTTATGAAAGGAAAGAACTCCAGGATGTCTTGTTGGGTAAAAACAGCAAGTTGTAGAATAATATGTGTAGTATCCTGTGTATTGTTTTAA
ATCTCAAAACATTTTACTTTTCTGCATGTAGATGGATAAATACATAAAAAGAGGTCTGAACAGTTGTACAGATAACAGTGATTGCCTCAATGGT
GGATAAAGCGAGTTAGCCGCTGCTGGGAAAACTGGCTAGGCATGTGTAGAAAGCTGAAACTGGAACCCTTCCTTACACCTTGTACAAAAATTAA
TTCAAGATGGATTAAAGACTTAAATGTTAGACCTAAAACCGTAAAAACCCTAGAAGAAAACCTAGGCAATACCATTCAGGACATAGGCATGGGC
AAGGACTTCATGTCTAAAACACCAAAAGCAATCGCAACAAAAGCCAAAATTGACAAATGCGATCTCATTAAACTAAAGAGCTTCTGCACAGCAA
AAAAAACTACCATCAGAGTGAACAGGCAACCTACAGAATGGGAGAAAAATTTTGCAATCTACTCATCTGACAAAGGGTTAATATCCAGAATCTA
CAAAGAACTCAAACAAATTTACAAGAAAAAAACAACCCCATGAAAAAGTGGGTGAAGGATATGAACAGACACTTCTCAAAAGAAGACATTTATG
CAGCCAACAGACATATGAAATAATGGTTATCATCACTGGCCATCAGAGAAATGNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNCCTATAGGGAGTATATTTATA
TATTATATTATCAAAAGTAACTATTTATATAAATGAACTTGATACAGTATTAATCTTAAGCTGTGCCAGTCTCTGTTGCTCTACTTTGGAAGAC
TATCCTATCTGCAAAACAAGAGTAATCAAATGTTAGCTTTTGTAACATTTTGAGTGCTATAGAACAAATAAAGTCAACTTTTCTGTTAAGTTTT
TTTGGTAACTTTTGATATAATTTCAAACTTACAAATTGTAAGAATAGGCTGAGGAGGGTGCATCGCTTGAGCTCAGGAGTTTGAGACCAGCCTG
GGCCACATGGCAAAACTCCGTTTCTACAAACGAAAAAAAAAAAAAAATGCCAGGCATAGTGGTGTGTGTCTTCAGCCCTAGCTATTAATACTTG
GAATGCTGAGGCCAGAGAATCACTTGAGCCCAGGAGGTTGAGCCTGCGGTGAGCTGAGGTCGCACCACTGCACTCCAGCCTGGGTGACAAAGTG
AGACCCTGTCTCAAAAAAAAAAAAAAAGTAGAAATAATACAGCAAATTCTTATATACTTTGCCTGTTTGACTAATTGTTTATGTTAGAAAAACT
TTTTAAAATTTTAATACATTGTTTCAGAGAAATGTGGTAAAAAGGGTCTGAAAATTTTGGATCCTTTAAAATATGACATTTGTGTGAGTGGTAC
AGAATTTTAGTTTTGAATTTTACAGTTTTATATAAAATATTCTTGGTAGATCGGCTGAACTAATGTTTATTGAGTATTTCCTGGTTTCCAGATG
TTGTGTTAGGATACTTTGCAAAATATTTCATGTAATCCAATTTCATGAAGAAGATTGTTATTCCTATTTTAGAGAAGAAGCAACAGACAGAGGT
TATTTGCTCAGGATTGTAAGCGAGCAGAGCCAAGTTTTCAGAAAGTCTTTATGTTACCAAAGCCCACATCCCTTTCTCTACAAAGCTGCCTGTG
AGATGATATATTGGAAGGAATTGATGAATACATTTATGTTGTCAAGAAGTTGATAATTACTCCTAAATTGGCTATGGTCTAATGTAGTGGATAT
TCAATAAAATTATCATGTTTGAATGCCATAAGAATTTATCTTCTGGCCTGTTTTGGGGGTTCATGAATGTAATTATTTTAGCTGCCTAACTGCT
TATGTGGGTTTGTATGTACTTTTGAGTGGTTCTTGGATATGTGTACACACATACATACACACACACAGATATACACATACACACCTTTATATGC
TCATGGGTGTAACTTCTTTATGTGTCTAGGTGTCTTTTTTTTGTGGAGGGGGGGCGAAGATCAATATGAATCCTATTTTGGCCCCCTGGATGCT
GTCACCAGAAATAACAGCCATATCCCATATATACCCTGTCCATGTAGCCCTGAGTTCTCCCAGCACCTGGCTTTTTGCCTTCCTTTTGTTTGTC
ATACCAGGCTCTCTTGGTCACCTTGGTAGCCTTGCTAAGCTTCTTGTAGCTATAGGTACCATTGGCTTAGCCCAGAAGAGATTCCTGCCATATG
TACTTCAGAGTTTGGTTTGCCACTTGAGGAAGGGGTCTGTAGGGAGCTTGTAGATCAGCACACATTCAAAACTGAAACCGGAAGTGGGCAAGGC
AAACCACAGGAAGATCCTGCCCGCAACTTGGTGACGTATCTATATAACCCATAGACACTTCCTGGCAATACCTTGTGAAACTTGCCTTTTCGTG
TATAGTAGTTTCCTAACTTCTTGTTCTAGATTAAGCTTCAGTGAAAAGTGATTAATATGATAGCAGTGTAGATAACTGTATGATAGTTATTGTG
CACATGACTTCCATGCAGCAACTAGCCCCGATTATGTTCTGTAGACTTTATCACTGAGGACTTGCAGTGGTATTTATGCGTTTACTTTGACTTT
GTCTTGTCACTAGCCATTGTCTTACTTATGTGTAATTGTACTTATATATAGTTCAGGTTTTATACCCACAAAAACCCTTGTCATATAATACCAC
TATACAGGATTGCCTTATTTATGTGGAATTGTACTTATATATAGTTCAGGTTTTATAGCCACACACACACACACGAAAAAACAGTTCTTTGCTT
CACTGAGTCTCGGCTAGCCACCCAAACTGTGTCATATTCCTACAGTCCTGTGCCCACTCAAGTGTTTCCTCACTGGTACTACCTCATTTTATCA
TAGTGCCTTGTTCTTAGGCACAACCAGATTTCTGGTAGGAGTTATTTAATGTGTTGATTTTTTTTTTGAGACGGAGTCTCCCTCTGTCACCAGG
CTGGAGTCCAGTGGTGCGATCTCAGCTCACTGCAACCTCCACCTCCCAGGTTCGACAATTCTCCTGCCTCAGCCTCCCGATTAGCTGGGACTGC
AGGTGCACGCCACCACATCCAGCTAATTTTTGTGTTTTTAGTAGAGGCGGCATTTCGCCATGTTAGCCAGGATGGTCTCGATCTCCTGACCCGC
CCACCTCGGCCTCCCAAAGTGCTGGGATTACAGGCATGAGCCACCGTGCCTGGCCAATGTGTTGTTTTTTTAAAGATTACCAATCGAGTGGGAT
TATAGACTCTTGGATTATTTTACAGATACAAGTCATTTGCCAGGACTGAGGAAAATTGCTTGACAACTCAGTTATCTCACCATGTTAATTTATG
CATTCGAAAACTCTGCTTAGTGGTAGATATTCCACCAGTCACCATATGCCTATTGGTACTATTTCTCATTCTCTGTCTGATTAATGTCACAAAT
GTGTAACTTTCCAGTGGTGTTTTCAAGATGACAGTTAAGGACCCACTAAGGAAGGAAATAACAGCTACATAACTTTTTTCTTTTCAGTAGTTCT
TGACAGTCCCCCCTCCTTTTTTTGTTTTTGTTTCTTCATACCCTGCAGCAATCTCATGTACTCTGAAGGGGGTAAATTTGAGACTCTTTGGAAG
GTCATGTCTCATAAAGACAGTTCTTCTCTTCCTTTCTCCATTCAGAACTGTTAAAGATAAATCAATGGGAGTGTTTTTTTCTTGTGTAAGATTT
ATTATATACAAAATATTTTTTAAGTGTGTGAAAACTGATTAAATGATAAAGGCTAAGGTAGCATGTTTAAAGCATGGTTTTCTGCTACTTTTCT
ACTAATCACTTAACCTCTCTGAGCCTCAGCTTCCTTCTCTACAAATTAGGAATAGTAGTTTTACTAATCATATACCATAGTTGTGAAAAGGTTA
AATGACTCGGTATACTGCCTGGCATTTTGTGCTCAGTTAAGTGTTGGTTCTAACATGAGCAGTAGTGATACTCATAAAGTATCTTTTCATAGTT
TTCTTAATGGATAACTTATTTGATAGAAATGTACTGGAGTATTCATACTTACATGTATATCCAGTTCTTGTAGTGTTCATCAGGTTAAACTGGC
AATAAGTGTTAATATCTCAAAGCATTAGAAATCAGTGTTACTCTGTGCTTTTTAAATTTCAAGTCTTGTCTCTCAGATTTTATTTTTTTATTTT
TTATTTTTTTTGTCTCTCAGATTTTAGAGCTATAGAGAACCTAGAAGATTAACTGGCTAAGGAGTTGCAAACTCAAATATACACAGACCAGGTA
TAATGCAGTAAAGCCCAGCAGAGATTATTATGATTTAGTAAGATACACCTGTCTGCAGTTTCCACTCAAAATCATTTTGCCAGATTTTGATTAT
TAAAGAGAAACCAGAAATACAAATTTATTTATTGTTAGCTCTCAGTTTCTAGATGGTCACAAATTATTTTTTAAATTTGGTGCAGGCCAAACAA
ATCTCTGGCTAGCCAACCTCACATTTGTATCCTCATTTTAGAGATGTGGAGCTTGAGGTATAAAGGTTGGACGTGTTTCCATGTATTTCCTATA
ATAGAAATCCATTGATTCTGTAACAAAGGACTCATTTTAAACAATGACCTCCTCTGTTATACCCAGTTGTTATATTACAGGTTATTTTTGGTCA
GGAGCTAATGGCTTGGATGAAGGGAATTGTATATAGATGCTAATTTTTAAGAATGTCTAGCCAAGAAAGGGCACTAATAGTTACTATCCATTGA
ATATTTACTCTGTCTTAGGCATTGTGCCAAGCGCTTTACCTTAGTTAATGCTCACAACAATCTTGAAGCATTATAGTTACCTCTTTTTACATAA
GAGGTTGAGAGTAAGGACATACAGATAGTTAAATGCAAAGTTCATGTTGAAACGCAGATGTCTGCCTCTGTAATGCATTCATTTAACTTGCTAT
ACTATACTAGTACTGTATATGTTTTTATAATTATGCATACCTTTTATCATATTGCAAAAATTGAAATTAAACTTTAGAAGAAAATTTCAAAGAC
CTTATGATATATTACCATTATATAGGGTTGTCTTATTTATATGGAATTAGATTGGCACAACAGCATTTTAATTTTACTCTAAACTAGTCAATAC
CAGAGACTATATCATATTTTTAAATGTGCATTAGATTCATTCTTTCTCACTCTTTTTTTTTTGTTGTTGTTGTGAATGTAAATGACAAAATGTG
CATATTTTATATCTTTTCATCTCCTGAAGTTCTACTGTTCTGTTTAGGTTGATATTTCTATTACTGGTTGAGCATTCCAAATTTCAAAGTCCAA
AATTCCAAATGCTCCAGGATGCAAAACTGAGCACCAACATGATTCTCAAAGGAAATACTCATTGGAGCATTTCAGATTTTCAGATTTGGGATGC
ACAACTGGTAAGTATATATAATGCAACATTTCAAAATGTAAAAACATTTGAAATCTGAAATACTTCTGGTCCTAAGCATTTCAGATAAGGGATA
TTCGATCTGTATTAGAACATTATGTCCTGAGTCATTCAAGCCAGTTAATGCCAAATTCTGTGGATTCTGTCTCCATAGTAGCTCTTCTATTTTT
TCTTTCCTCCTATCTCCCTTTGTTACCTTAATGTAGGCTCTTCAGACTATCAGAGTACTTTTCCTGAAGTATGAACCACACAGAAACTTTTAGC
GACCCTTGTTGCCTACTGAATAAAGTTAAACCTTAGCTTGCCATTTTAGGTTCTCTGTTTGATACTAGCTGTCCTTTATAATCTTTTCCCTCTC
ATTCCCCCTTCATCTCTCTTTATGCTTTAGTCAGTTTATACTATTCACTGCCTCCTCGATGGTGCTCTTCTTGTTGCCACCCTCCAGAACTTGT
TCATGTTATGCCTTCCTCCTGCAACGTTATCCTCCGATCCCCAATTTCTGTATTTTGAAAATATATATCATTCAGAAGAGTTATTGTGAAGATT
AAGTAAAATAATATATATGAAGTCAATGAAAAATCTGTGTAATACATGTATTAATTTATTCAGTAAGTGGTACCTATTATTATGATTTGGCTTT
CTGTCACCCCAAATAATCCTTAACTTTGGATCATCTTGTCCAGGACCCTGGCTCTTTGCAGTTTTTCTAGGGGATTAATCCCACTGGGACCCAT
GTTGGCGCCTACCCTTGTACTTCAGGTAACCTTTGAAGCAGACCTGGACCTACTACATCTCTTTCAGATGGGAATAGTTTCTCAGCACAGCTGT
ATTCAGAAAGACCTTAACAGTTGAATACAAAGACTCTAGGCTTTGGAGTTTGATTTATGCCAGATAACCTTACCAGCTATGTAACTGAATTTTT
TTTTCTTAATTTATGAAAGGGAATAGTAATGCTTATCTCACAGGGTTGTCGTACCGATCAAATGAAACATTTGAGAAAATATTTAACACTGTCT
TGGAGCATCTAACTACTTAATAAATCTTTATCTTCTTTTCTGTCTTATCCCTGTAGTGTCCTCCCCTAGGACATGAAGAAACATACATGCAAAC
CTTCAGGTATAAATCCATGCACATAATCCTCAAACATCCACATAGGCACATAGGTACATACTCACATATGTACATACTCACATACATGAACAGA
ACCATATACATCAGAATCTCTACCTTTGATAGTAGCCTGATATTGGAATTTTTGTCCTAGATAAAATTTATATGGTAGGATTTGAATTTACTGT
CTGATGACTGTTACATGGAATCATTGATCTTGTACATAGAGAGTTGGCTGTTTTTCCATTTCTGTTAATCTTAACAGTTTTGATTTGATGGTGT
TTATCTTTCGCGCTTTAAACTATTTACTTTTTAACAATATCTAAAAGCTGCTGTTTTCTATAGTTACATGTCTCTCCTCTTTTTTCAGGTATTT
ATGCAGAAGTTACTTTCTCAATGAACCCTTCCTTCAGCTACCTCATTTCTTGTGGTGCCCATACTTATTAGTATTAGGCCATACATTTTTACTT
ATTTATCTTATTTGTTGTCGATCTCTCCATGTGTGTTAGCCTAATAAGGACACAGACTTTTTGTCTGTTTTGCTCACTGTCATATCCTAGCAGC
TTAGTAGGTACTCAGTAAATATTTGAATGTGTGAACTTCAGCACTGGTTTATTTTCAAAACGTCTCTGTATTAATACAATAAAGATTCTTTAAG
TGCTTTTGGTAACTGTATACAGGAGACTTTTTAAAAAATCTTTGGAGTAGTTGACATAAATCAACTTAAATGAAATGGAAAAAAGAAGTTTACA
TACAATTTTATGGTTTGTGATGTGGTATTGGAACCTCTTTTTTGCCCATGCCAAAGTTATATACACACGCATTGATACTTATTCAGCAGCTGTT
TCATTATATATTTTATGCTAAGGATGGTGCTAAGAATTGGTCCCCTTCCTCAAGAAGTTTATAGTTCAGTGAAGAGACAACCAAACTGCTTTTG
CTTGGTTGTGTCTCATCTTTACCACCTCAAAACATTAGACAGCCCTGAGGCTCACTCTTCAAATTTCTTTTCTATATTTGCTCCCATTGTGATT
TTGTATTGTCTCTTGGTTTTCTAAATTTTATGTACTTACTGATGATTCTCAAATTTTTTCTCTATCTAGGACCCTTTGTCCTTGGACAGATTGT
CTGCACTTGGATGTATAAGGCATCTCAGATTTAATATTCAATACAGTCTCTTGAATTTGGTCCCCAACAAGAGGCTGTGTCTCTAGAAATAGCA
TCTCTATTCATCCAGCTGCTCAGACTAAGACTCTTAGAAGTCATTCTTGACTACTCTATTTCTCACACCCTGCAGTAAATCCTGTTGGCTCCAT
CTTCAAAATACATGCCCAAATGACCCATTCTCTCCAGACCCGTGTCTGCTCTGCCCCTCCCCTGACCTGCAAGTTTTCGCTCATTGAGATTACT
ACAGTAGCCTCCTATCTAGTCACCTCCTTCTACCTTTCCCCTCTGCACTGATTTTAAAACAAAAAATCTTGCCATTTTTATGCTGAAAATCCTT
CAAAACCTTACCATCTGACACACAAAAAAAGCCTAAGACTTTCCCGTGCCTGTAAGACCTTATTTAACCTGTCTCCTCCCATACCCTTACTCCA
TCTCTTACTATCTCCTCCCTCTCCCCACTTTGCTTATTGCTTTGTAGCCATACTGCCCTCCTTGCCACTCCTCCAGTGTGCCAAACTATCCTGT
CTCAGGGCCTTTGCACATTTTATTCTTTCTGCCTGTCCGTCTCCTTCCCCAGATATCTGTATGGCTTATACCCTCAATGACTTCCTTCAGGTAT
CTGCTTAAATATTTTAATTGAAAGGCCTTCCCTGCTTCCCTATATAAAATAACATTGACCTCCTCCCTGTCTCTACCCTCTGGGCCTGCCATTC
CCTAACTCTCTTAACCTGCTTTCTTTTCTTGAATAGCAATAAATATTTATTTCTTCCATGACAATAAATACTATATGCTTATTTAAGAGTTTGT
GGTATTTGTCGTCCCTGTATATATTCTACAACAGTTTCGTATGGGCAGAGACCTGTTTTTATTCACTGTTATATCCCCACCACTTAGAACAGTG
CCTCGTGTGTTTAGGCACTTAGTAAATATTTGTTGAGTGAAATGCAACAAGTACAAAACAATATGATCACTGTAGTAGAGATGCAAGGTACAGT
AGAAACACATGGGAAAGAGTGACATGACTACATAGGAATTGAGCAAGACTTTGCAAAGATGCCCTTAAGCAGAGCTCTTTGGAGAGAAAAACAG
GTAGGGCATTTCATACAAAGAAAATAGCATTGCAGATACACAGAGGAATGAGAGAACAGTGTGTTCAAGGAACTAAAATATAGTACTGCTACAG
CATAATTTGTGGGGGTAAAGATGAACTAGATGAGGTTGAACATGTAGTAGATAGCACCTGATCATGGAGGGATGGAGGGTTTTTGTGCCAGACT
AAGGAATTTTAGATTTTGTCCTGTCAAGTGGGCAGTGGTAGATAGTGTGACCCATTGAAGACTTCTAAGCAAGGGATGTTTTATTTATATTTAT
TTATACGTGTCTCTGTCTTTGTGTGTGTGTGTGTGTGTACGTATTAAAAAGGTAACTAGCAACATTGTGAAGGTTGAGTATCAAAGCTAAACAC
AGCATTTTATTTAAGATATGTATGATTTAGGAAGATATGATGTAGAAAAACAATTACTATCACATGTGGGCGAGAGGATATTTCATTTTATATT
TGGACAAAATCATGGATGTATAGTGGGAACATGTTCTTGGCATACTTGCAGTACATTTGTGAAAGAGAAGAAATGCACAATGGACGATTATTCT
CTTTGTCTTTGAAAATCCACCCTTTAAGAAAGAAGACTTTAAAAAATTAGTTAACAGTTATGTTGTAAGTCGTAATGTACACTAGAAAAGTGCT
TAAGGAGTACCAGTTATGAATCAACAAGAAAAGATGTTACAGAATAAAATAAATTGTGCTTGAATGTCAACCTCCTTTCTTCGTAGAGGAACAG
TTGGTTGTCATTGATATACCTCCTGATCTTGATTTATTGTTGAAGGAAATAGTGACAGTTGTACACACAATCAAATCTATACTGTTTGCTGTGG
GAACAATGTATCTGCCTTTCTAGGACAAATGTCTGCTTTTGCATATTCCACGACTCCGCATGCACAGAATTTTTGAAATGAGGGATGAACTATA
AAAACATTTCTTCCTTTCATTCAAATCCAAGTAAAAACCATTTTTATTATTTCAGCTGGCTTGCTTGTGTCATATCTTAGCAATATATTGAATT
TCTTGAATACTTTATTCTTATTAATTTAGGGTCAAACTATGATTTGTGAATTTAAGGATAAAATAGAGGCACCCAAAATGGTGCACACGATTTG
AATGTTAAGAATTTGGATTTCGCACTCCCAGCCAGGCATGGTAGCTCACGCCTATAATCCCAGCACTTTGGGAGGCCAAGGCAGGCAGATCTCT
TGAACCCAGGAGTTCAAGACCAGCCTGGGCAACATGGCAAAACCCTGCCTCTCCATAAAATACAAAGCAAATTAGTCAGGCATGGTGGTATGTG
TCTGTAGTCCCAGCTACTCGGGAGGCTGAGGTGACAGAATCACGTGAGTCCAGGAAGGCGAGGCTGCAGTGAGCCATGATTGTGCCACTGCACT
CCAGCGTGGGCGACAGAGCGAGACCCTGTCTCAAAAAAAAAAAAAAAAAGAAAAAGAAAAAAGAATTTGGCATTCCAAATTCATGAAAATATGA
ATACTGTATTATTGAATATATTTTTAAAATTTTATCCAAATGCTACAGAAGATGAAGAAATATTTTGTGAAGGTAGATACAACTATATATGGAG
TTAAGATCCATTTCCCAAGTTAAACGATTCCCTTTTTAAGTGTGCTGCTATTCAACTCACTATCTGATGGAACAATGAAAATAATTTTCAGTGA
AAAATTGCTTGGTAATTTCTGTGTCCATGTTTGATCCAGATACCCACGTGTAGCCGGTGGATTTACCCAACCATCATGACCAGTAACTCAAAGT
GGGATTTTCTAATTTGTTGGAAACAAAGGTTAGAAAAGAATTGAGATTAGGTGACAGCATCAACTCTAGTTAGTTTAAGCAGAAAGAGCTTTAA
TAAAAAAATTTTAAGTCCCTTACAAAATTGTTGACAAGGCTGAAGAAACAGACTCTAGATCTAGAAAAAGCCATGTTGCATCTAGGCCACCAAG
AGAGTTATTGCCTCTTTTAGGATCAGAAAGACTTTTGCAGAATTGTGAAACTGTAACTTTTAGATGCCTGTCTTAGAACCTTACCACCTCTGCC
ATGATCAGGAGTCTACTACTGTTAGGAAATAGCCATTAGCAAGCAGCCACCTTCCTCAGTAAGCCGTTCTTGTTGCTGGCCCTAGAGCCAAGCC
CTGCCTACCACAACCTGTACTGCAGAATGGATGCCTTCTGCCTTTCTTAATATTCATGAAATCTGTGACTGAATACTAGGACTTCAACAAATAC
TGCTGTGAACATGAACATCTCCATGACAATGTTTCCTAGTAAAACAACCCAAACTCAACCTCCCTTCTCTTCTGTCCTTGAAATTTCTAGTGAG
TGCATCTGGTTGGTGGAACTTAAATCACATTTACAACTGTAGCCACAAGAGAGCCTGGGAAATGTATTTTTTAACTTTCTCTTCTCTGCAGCAC
AGGAAGACATACACTGAAGGAGGTTGAAATGGATGTGAACAGTAATCTACCATATTTACTGCAGGTTTACAACCTGGTTCTCCTTAAGGCTGGC
TGTCATTAAACGGGCTATAGATAAGTTGAGAAAGTTTATAAATAATACCGTCAAACAATGTTGAGAAACTCATTTGTTTCTTTGTATATTTTTA
TTGTTTTATAATGACTACATTAAAAAAAAAACTAGAATTTGTCCTTCACCTCTATGATGAATTAAGTGGTCTGTGGATACCGAAAGTTTCCTCA
CGTTTAAGATGTGGTTAGTGGATAAAAAGACCAAAAGAGGGAGTATAAGAAAAACCCTCATACAAGGTTACCTCAGCTGAGGAAAGGTTAGTGT
TGTCAAATAATGAAGAGTGGTCAAATAAGGTGGGACTGAAAAGCAGTGTTACGATTTGGGCACTTAATATTAGTGGTTTTTTGGGTTTTTTTTT
TCTTTCTTTACTATTCAGACAGGGTCTTGCTCTTTTGTCCCAGGCTGGAGTGCAGTGGCAGCTCACTGCAACTCCCATCCCCTGGGCTCAAGCC
ATCCTCACACCTCAGCCTCCCAAGTAGCTCGGACCACAGGCATGCACCACCACGCCTGGCTAATTTTTTGTATTTTTAGTAGAGATCAGATCTC
ACCATGTTGCCAAGGCTGGTTTCAAAATCCTGTTCTCAAGCAGTTTGCCCACCTTGGCCTCCCAAACTGTTGGGATTACAGGCATGACCACTGC
GCCCAGCCTGTTAGTGTTCTTCAAGGTTCTAAACAAGAAGTAGAGAAGTAGGATAAGAGGGCAAGTTGCACGTTTAGGCTGTGAGTTGAAGGGA
GCACTTGAAAGGAGAGATTATAGAATAAGTTTTCAAGAAATTTGGCAATGGATTTTTTTTTTTTTTGGTCCAAGGCGCATGCTCATTTATATAC
CAGAATTGGCTAGAGTTCATGAGGCCCTGAACAAATATCACACACAAGCAAGTACAGTCTTCCAAAGTTCTTCACTTTTGTCTTTTTGTCTAGC
TTGATGTAAAGTGTGGCTCTTAACATATTCTCTTTATGCTTAGAGATATAATAGGCACAATATTACTTTTATTATATCTGTAACCATTACTTCC
TTTTTGATTATGTTGTTTATAAAGTGCCAATTAATCACTACTATAGTTTGGAGTTACAGTATCTGACTTAACATATTTGTACAATTGATTGTTG
AGCCGGGCATTGATTAGATGAATTAACTTTCAACAAAGTTTATTTGAACTTCAGTCATTGGTGGATATTTAAGAAGTACAGATTATGATAACTG
CTTTTATAGGAAAGGATGCTTGGTACATTTTTTCATATGAGATAGATCTTACATTTTTATTTATTCTGGATAAATATCTGATAATGTCAGGTTC
ATTGGATTCTTCAAAATAACCTCATTGTCTCTCTCTTAAGAGCGAGAGGGAAGTAGAATATGTGTAAATTTATATAATGTTGTTTTCTCAAAAC
TGCACCTATGTGAATGAAACAGAAACATGTTCAGGGACAGTTAGTGTTTCAAATTCTCCCAATTAGATATGGAGCATATTTATTATGGAGCATA
TTTATTAAGTAGAAAATTATTATGGTCTAATTCCAAATTTTTTATATTTTAGGTCCTATATTGCGACCTAGTTGGACTTTCGTTTAAAAAAAAG
TCTAAATTATGCAGAAGGGAGACCTCAAAGAAAAAGGAGAGACTGAAGAACAAGAAGCCTGAAAGTAATGAAAATAGAATTGCTAGACAGTGTC
CTGGAGGGACAAGAGGGAATGGGCTAGAGAGAGCACAGGTAAATGGATGAGCTTTGGATGAGAAGAGGAATAACTAGTCTTTGGAGGAAGAGGA
AAATGGATGAATACCGGTTCAGATATAGATTATTTGTCTTAGTGACCTGGTTTATTTGTAAGGACTAACCCATTTTAGGCAGTTAATTAAATTA
ATCATTTAAAAGGTGAGATTCCTCAATTCTGACTTGAGATGGACCTAAATTTGTTTATTCTGTTTAAGTAGTAACTTTATTTTTTTAGTGACAG
ATTTAAGAAGTATCTTACTGGTACTATTCCATTGGTTTTCTTATTTTAATTTTATAAATAATGTGTTTACTGAGTTTTTTTATAGGTGCTTCTT
AGGTTTCTAATAGACTTCTCTAAAATATATTGGTTTAAATGGCCTTTAAAGTTGATAACCAGAAAGATACTTGAGTTTATCTAGTCCAAACCTT
CATCAGGAAAGCTGTGATTGAGTGATTTGTCTGAAGTCATACAGCCACTTAGTACCAGAGTAAGAGAGCAATATTGGTTTTATGGTTCTTGGGG
TAATCCTCTTTCCCTTATGCCAACTGATTGCATAAGGGAACTAGTGTACTGATTTCAGCGCCCATACTTGTGTATGTCCTTTGAGCTAGAGTAT
CAAAGGACTTCAAAACTTCAAAACTAGACTTTTTAGGTTATTTAATATTCCTTCAACAAATCTTTCTCAGCTGTCTACCGTATGACAGTCACTG
TTTAAGATCTTGAAGATCAACAAGTTCTTGGGCCTCAAGAATGAGGGAACCAAAAAATAAACACTGAATTAAAAATGAACAGAAATTTAGATAA
TCATCAGTCCTATCAAAGAAACAAAATGCAGAAATAGAATATGCTAGCAGAACTATGTGCTTTAGATAGAATAGTCAAGGAAGATTTCTCTGAG
TTAGTCATATTTGAGCTGAGACTGAATGATAAGGCACTCGCCATTGAAGATATCTAGGAGTGTAGCATTCCAGACAAAAGCCCTAAACTTGAAC
ATGGTGTGGCACAGTTGATGAATGAAAAAAACTAAACAGTGACCGGGGTTGGGGGAATGGAAGGAGAAGAGATCAGAGAGACCATTAAGTAGGG
CTTTGTAGACTGTGGTATAGAAAGATAATTTTATTCTAAGTGAAATGGTGTGGTATTGGAGGAGCAGAGTGAGTGAGTAACCTAAAGAATATGT
TTCAAATATCACACTGCTTGATCGAGAATGGATTGTAGGAGGGCCAAAAGTGGAAGCAGGGGCACCAGTTAGGCAACTCTCATGTTTGTTCGAA
TAAAAGATTGTGGTGGCTTGGACTCGGTCATTCTGGTCAAGGCAAACAGAACTGGTTGGATTTGGCATTTATTTTGGACCTCTGCTGGATTTGC
TAAGAGACTGGGTATGGGAGTTTGGGGAAATAGAGGATGGTGAGAGTAAGAGAGAAAAATGGTAGACTGGGGAAAGGGCACATCTTGGGGAAAG
AAATCAGATGGTTTTTCTGGGACTTGTTAAATTTGAAATGCCTACTGGATATCCAAGGAGACAGTTAAATAGTCACATCTAGTGTTTGTTGAGG
AGAGGTTAGACCTGGTGTTGCAGATCTGGGAGGCGTTCGCATTAACACCATATACTGCAGAGTTGGCAAAGAGAAGGAAGAGAAGGTGGTTTGG
AATTGAGGCATTGCATGTTTAGAGGTAGCCAGGGGGAGAAGTCAGCAGAGAGCCAGTGACGTAGGAGAAAAGTCAGAAATACTGTGTTGTGGAA
GCCAAGAGAAGAGACCATTTTAAAGAGCGATATTTATGTTCACTTGGGTTAAATCATCCTGCCACGCCGGGTATGATGAAAACAGAGAAGTGAC
CGTTGATTTGGCAATGTGGAAGGCATTAGTGTCCTCAGCTAGAGCAGTTTTAGTGGCATGGTGGGGTCAGAAGCTGATTGGAGAGGGTTGAAGA
ATGAATGGAAGGTAAGAAGAGACAACGACTAGAGATCATCCTTTTAAGAAGTTTTTTTTCCTTTTCTTTTTGTGTGTTTTTTTAAGTTGAGAAA
TTCCCATCATCGTTTATATGCTGATGGCAATGATCGCTAGAGAGGAAAATTAATGGTGATTTGGGAAAGAAAGATTAATTGTGGAGTAAAGTCC
TTGAGGTAGCACTGGTGGAATCGCATACAGACCACAAGTAGAGGGATTACCCTTTCATAGGAGTGGGGATGCAATTACGTAATATAAAAAAGGG
ACCTCAAGCCACTTTACACACAGCCCCTCCCTTCAGTCTTAACAATCTCCTTTAGACCAGCTTGATCTTTCTGTAATCACTAGTGTTTTTTTTT
TTTTCTGTGCCACATTAATATGTTCTTTCAGATTGAAGTCATCCTATATAAGGCTTAGTATGAAGCAGTTGATGATATTTAATAACAATTTTAA
ATTTTATGCCAACAGGTGAAGCAAAAAATTTATTGCCATATCTTGGACCCCACAAAATGAGAGATTGTTTCTGTACCATAAATTTGGACCAGGA
AGAAGTTTATCGTACCCAAGTTGTGGAAAAATCTTTAAGGTTCGTAGAAAAAATTGGTAAATTATAATTTTTACTATATAGATTGGTAAATATT
ATGCATTTTCCAGATTATAATAATCAAGAAGACAAGACAGAAACAGTGCTTGCTTTCATGTGGCTTTTCATTGCCTCAAAATGTATCTTCCTCT
TAATTGTTCCTAATTTTATCTTTGTTTTCTGGAGAAAAGTTTTTTCACATGACAGGCTAAATATTGCCTATTCGTTTGAACTTTCCTCAAAGTA
CATCTTTATTTTATTTATCTAAAGAATAAGTTACTTAAAGTTCATAGAAGTTGCTTAACTTAGTAGTGCATTATCAGGGAATGAATTCTCAGAT
AGATGTCTCATCTTTGAAAGTGTAATTGCCCTTTGAGAGTATCATATTAAATTCTAATTCATAAAACTGCAATATACCACCTCTGCACCGTAGC
AATGTGAACATAATTCTAGTGCAGAGTTGTGCTGAGAATCTTGTTCAGAGACAAAAGTCAAAATAAGGAGGCCGGGCGTGGTGGCTCACGCTTG
TAATCCCAGCACTTTGGGAGGCAGACGCGGGCAGATCACTTGAGGTCAGAATTTCGAGACCAGCTTGGGCAACATGGTGAAACCCCGTCTCTAC
GAAAAATACAAAAATTAGTTGGGCGTCGTGCTGGGTGTCTGTAATCTCAGCTACTTGGGAGGCTGAGGCAGGAGAATCGCTTGAACCCAGGGGT
CGGAGGTTGCAGTGAGCCAAGATTATGCCATTGGACTCTAGCCTGGGCAACAAGAGTGAAACTCCATCTCAGAAAAAAAAAAAACAACAAAAAA
ACAAAAAACAGGGTGTGGGGTATTATTGGAATGAGCATGTGGTGGATGAAATATTGAATGCCATGCCTTATCTGAATCATATTTTAGGTCAGTT
GTATTTTAGGGATCTGTTTTTTTTTTTTATGCCTTTAAAAAAATATCTTTCTTTTACATTTGCCTTTGAAGTTTCCCTTTATGTCTTTATTTTC
ATTTTTTATAATTTTAAATATCTATTGAATTCTTCTTAGAAAAGGTATTGTATAAAACTGAAAATACTTGAGTAGCCCAAAAACATTTCAAGGA
GTAAACTTGAAATCTTTAGTCTTAATTCTCATATTGTTCTTATTTGGCTTTAGAATGTACTTTAAACATAATATTTGAATCTGGATGTGATATA
TAAAGGAAATTCTGCATTTATAATTGTTTAGAAAATAATGCAAAGAGATACATACCTACACATGTCTTGTCTTTCATATCATGTTTTCATATCA
TATTTGACTAATAGTTCTTTTTGCATTCATTTCTATGTTCAAATTAATTAGAAGGCTCTACTTACTTTGTCAGACCAACTTTTAAAAGTTTAGT
TTGCTTACATTTGAAGCGAAAAATATACTCACAAAAGTTCACCAGCCATTTTAACAACTAAAAACCTGAACCAACTGTTTTCTAGACAAGATCA
GATATGACTTCTGTAGGAGTATTTTACTTTATATTTCAGCTTCTCATTTAGTTATGACATCATAAAGATACTGCATTTCATAGCGATATTTCTA
GAGAATCTTTTTGTTCTTTAAACTTTCTCAGGAAAGAGGTACACTTGACAAAAGAAACTATCATTTCAGAGATTCGCGTCTTCATAGAAGAATC
ATAGAAGGATAAATATATCAAAATGGCATTCTTAAAATGAACTTATAAAAATAAGCTTTGCAACATGAGGATGGGCAAAGATTTCTTGCTTTGA
ATGCAAGAAGCACCACTAATTATAAAAGATGAAAATTGGTAAATTAGATTTTATCAAACTTAGAGGCTCCTTTTCTTTAAAAGACACTGTCAAG
AAAATTAAATGGCAAGCCACACCTGGGAGAAAGTATTCTGACAAAAGACTTGTATCCAGAATTTATTTAAAAACAAAAACCCTGATAACTCAAA
AGAAGACCGATGGCCATTTAAAATATGGGGAGAATACTTAAACAGATACTTCAGAAAAGATATGGCCAAGAGCACATCAAAAGGTATTGGACAT
TGTTAGTCATCAGGAAATCAATTTAAAACCATTTTTGTCACTATAGATTCTAGAACATAATCTGCAGTGATAGAAATCATATCACTGGTTGCCT
GTAAGAATATGGTGGGAGTGATTGCAGAGTCACAAGGGAACTTTCTGGGGAGATAAAAATGTTTTGTATCTTGATTATAAGGTAGAGATTACAT
AGGTGTATACATTTATCATGTTACATTCACTAGAATAGCTAAAGTTAAAGGCTGTCAGTACCAAGATGTGGAACAACTGGAGCATTGCTGTTAG
GGGTGTAAAACAGTACAACCACTTTGAAAACAGTTTGACAGTTTCTTGTAAATTACACATTCACCTGCCTTTTAACTCAGCAGTTGCATTCTCA
GTAATCAAAAGCATGTGTCTAGAAAAAGCTTTATATAAGAATGTTCATAGCAGCTTTACTTTTGATAGCCCAAACTGGAAACAACTCAAATAAC
ATCACTAGGCGAATGGATTTGGAATGCAATGGAATACTATGCAGCAATTAAAACAGTTGAACTACTATGTACACAACAACATCGAAAAAATCTC
ATAGACATTAAGCTGAATGAACAAAGCTGGACATGAGTACATACTATATGATTCTATTTATGTGAGATTTTAGGATAGATGTATAGTGATAGAA
AACACATCACAGGTTGGGGCAGGTATGATGGGGTAACTGAAGGAGAGGCATAAGAGAACTTTCTGGGGAGATAGCAGTGTCCTTTATTTTGATT
GGGGTGGAAGCTACACAGTTGTATAAATTTATCAGAAGTCATCAACCTCTATACTTTAAATTGGATTTTTATTGTTTGTAACTTATAGCTCAAT
AAAACTGATTTAAAAATACCTTGTTTTAATTTTTTAAAAATATTTTTTAAAGTTGATTTTAAAATTGCATATTTTAATTTTTTAAAAATAGCTT
AATTTAAAAAAAAATTTTGAAGTAGCCTTTCACATAACATTGGCTGTTTTAGGTATTATGTATTGTAAATAATACAGCTTTACACATTTCTAAG
ACAAGGATGGTATGAATGACACTATCCTCACCTCTCAAATGGTCCACCTTAGGTGACCATGACCCCAGGCAGTTACTGTATCTTCTATCCACAG
GACTGCTGAAAAGCTATTTGTTCAGAACTCATACTGGCAACTTAGATCCCTTCAGTGTTCAGCTTGGTTTCTATAGAACTTAGTTGAAATTTAG
CATTGAGAATTATTTTGGGCCTTTTCAAATTCTTTTCAATGGCATTTTAGTCCATAATTTCATTAACTTTATTGAAAGTGATATTATTTGGCCA
GGAACATTAGCTCACTCCTGTAATCCCAGTACTTTGGGAGGCTGAGGCGGGTGGATCGCAAGGTCAAGAGATTGAGACCATCCTGGCCAACATG
GTGAAACCCCATCTCTACTAAAAATACAAAAATTAGCTGGGCGTGGTGGCATGTGCCTGTAGTCCCACCTACTCGGGAGGCTGAGGCAGGAGAA
TCGCTTGAACCTGGGAGCTGGAGGTTGTGGTGAGCTGAGATCACGCCACTCGACTCCAGCCTGGCAACAGAGCAAGACTCTGTCTCAAAAAAAA
AAAAAAAAAAGTGATATTATCATTCTCAGCAAACTATCGCAAAGACAAAAAAACCAAACACCACATCTTCTCACTCATAGGTGGGAACTGAACA
ATGAGAACACTTGGACACAGGAAGGGGAGCATCACACACTGGGGCCTGTTGTGGGGTGGGGGGAGGGGGGAGGGATAGCATTAGGACATATACC
TAATGTAAATGATGACTTAATGGGCGCAGCACACCAACATGGCACATGTATACATATGTAACAAAGCTGCACGTTGTCCACATGTACCCTAGAA
CTTAAAGTATAATAAAATATATATATATTAAAAAAAGAAAGTGATATTATTTGAGTATTTATACAATTATTATTCAAACTGATATTAATTATTA
TTTATTTTATATTATTTGAAGTAATGAGCTTTCCTTTTCTTTCTAGCCCATTTTTCAGTGAAGAATTTTACTTTGAGATTCCAAGAACTTTCCA
GTATTTGTCTTTCTATGTTTATCATAAGAATGTTTTACAAAGAGATCTCCGTATAGGTATGTACTATTCATAATTATCTTTAATCACAATGTTA
ATGTTTATATTCATTCGTTCTCTTACTATAGTTTTGAAAATTGCAGAATAATATATGCTCAACATCTCTGATCTTTTTCTGTAGACTACATGTT
CATTTGTGAAATTTCATTTTTAATTTGTATGTTCAGCTTATATGACACTTTCTCAGGGAGGCCTTTCCACCTCCCCTAGTCACTATGTCATATG
TTTTTATAATTCCTGATGGTTCCCTTTCCATGGTACTCATTATGCTTATTCATTAGTTGGCTGTTTCATATCTAGGTTCTCTAGTTAAATGTGA
AATTTAAAACGCCAGGGACTCTTTTTTTGTTTTTTTTTTTCACACACAATCTTCCTCTATCTCCCAGGCTCGACTGCAGTGGTGCGATCTTGGC
TCACTGCAACCTCTACTTCCCAGGTTAAGCGATTTCTCCTGTCTCAGCCTCCTGAGTAGCTGGGATTACAGGCGTGTGCCACCACGCCTAGCCA
ATTTTTGTATTTTTAGTAGAAACAGGGTTTCACCATGTTGGCCAGGCTGATCTTAAACTCCTGCAGGAAATCCACCTGCCTCGGCCTCCCAGAG
TGTTAGGATTACAGGCATGAGCCACCACGCCTGGCCGGGCAGGGACTCTTTCTTACGGAGCACCTTACACTTACTAACTGTTGCGTAAGTATTT
GTTTAGTGCTTCAAATAATGTTTTTATAGAAAGATTGCACTTTTGATCCTTCCAAAGAACATCTGTTGTTGAGGTTTTTCTAATAGAAGTAATA
ATTCACTTTAATCTATTTATACATTTTCTTCCTGTCTGTATTAGTGACTGTATTAGTCCATTCTCACACTGCCATAAAGACATACCTGAGACTG
GGTAATTTATAAAGAATATAGGTTTATTGGCTCACAGTTTTTCAGGCTGTACAGGCTTCTGCTTCTGGAAGGCCTCAGGAAACCTACATGAGTG
AAAGGCAAAGGGGAAGCAAGCACATCTTCACATGGCTGGCAGGACAGAGGAGAGTTGGGATGTGCTACACACTTTTACAATAACCAGATCTCAT
CAGAACTCACTGTCATGAGAGCAGCAAGGGGATGTCTGCCCCCATGATTCAGTCACCTTATGCCAGGCCCCTCCTCCAACAGTGGGATTACAGT
TTGCCATGACATTTTCGTGGGGACACAGAGTCAAACCATATCGGTGACCAATAAATATATTTCTCTTTTTTTTTGAGACGGAGTGTCACTCTGT
CGCCCAGGCTGGAGTTCAGTGGCGCGATCTCAGCTCACTGCAAGCTCCGCCTCCTGGGTTCACGCCATTCTCCTGCCACAGCCTCCCGAATAGC
TGGGACTACACGCTCCCACCACCACGCCCGACTAATTTTTTTTGTATTTTTAGTAGAGACAGGGTTTCACCATGTTAGCCAGGATGGTCTCGAT
CTCCTGACCTCGTGATCTGCCCACCTCCCCCTCCCAAAGTGCTGGGATTACAGGCGTCAGCCACTGCTCCCGGCCAATAAATATATTTCTTAAA
TAATTGTCAGACTAGAGTGTGATCTGTGGGCATGAACTAGAACAGAGCTAACTGGCCACATATGTGTCACATTCCTGATCTTTGCACCACTATC
TTTGTATTTTAATCATAGGCAGAGGTCTAATTTACATTTATTCTCTTTCTTTTTAGTATCCTAAATTTCATCTCCCTTTCCTTTTTTATTTGAA
ATATGGTCAAGTCTCCTACAGCTTAAAAAATTAAGCATTATTTGGCCAGGTGCAGTGGCTCATGCCTGTAATCCTAGCACTTTGGGAGGCCGAG
GTGGGCAGNNNNNNNNNNNNNNNNNNNNAAAAAAAAAAAAAAAAAAAAAAAAAAAAAATAAATTAAGCGTTGTCTTTGTCCTCTTAACTAACTT
CTGATGTTTCTCTGTGTTTGATATTGAACTCCTTAACAAATCGTCTGTGTCAGTCCTCTGCTTGCTACTTATTTCTTTTAAAATCTCTATAATC
TGGCTTCTATGTTTTTTTGATAAACCTTTCTTGAAATTTGCCAACATCATTTTAATCAAATAACCTTTAAAACAATATGTTTTTCTTAATTATA
AAACTGTTATATGTTCTTAGTATTTGGTAGTGATTCTCTACAGAAATAACTACAGTTAATAATTTGGTGTATTTCTTCCATTCTTTATTTCATA
CATTTGTATTACTACATTTTATTCAGATTTTTTCCTTTTCAATTAATACCATAGTGATCCTTTCCCCATATCATTAATATTTGTTAAAACTATT
TGTTACATAATATCCTTTATAGAAAAGTGTTTTATTTTAACCACATCCTTGTTGGTGAACATTTACATTTCCAAATTTTGGGCATCCTCAGTGG
TGTTGCTATGAACAAATCTCCTTTGGCATAAATTTTTGCATGTCTCTTTTTCCCCAGGATAAATTCCTAGAAGTCAAAATACTGAGTCAAGGGG
TATGAACATTTTTATGATTTCCTCTGTAAATTTCTGTATTGCCCCTCTGCCCCACAGAGTATCATACTAATATGGACGACATAAATGTTGTACT
AATTTACATTTCCTCAAAGATGTCATGGGCATAACCATTTTACCACGCCCTGGCCATTCTTGCATATTATCATTGAAAAAAATTTGCCAATATG
ATAGGTGAAAAATAACACATTATTATTTTAATGTGTGTTTTCTGATTTTGAAGGAATTTGAGGAATTTTTCATATGTTTATTGGTATTTACATT
TTTCTTCTGTGAATTGATTGCTCATTCCTTTGCCTATTTTTCTTTTGGTTGGAGTTCTAACATTTATCCTTAAGCCTTCTTATTTTTAGATATG
TTAACTTTTGTCAGCTATATACATACCAAATACTCTCTCTCACAAAAGGCATTTTCTTAGTTTTTATTGTTATCTGCCTCTGTGTCACATTTCA
CTCTCTTCATTGCTTATTCGGTTTGCTGAAAGTGAAAGAAAAGACATTTTCATGGTGGTGTATTTTTTTAGATGTGTTTCTCCCTCTGATGCCT
TCTTATATGCAGACTCTTCTCTTTGTAGCAAACCGTTCATTATAGATCACCCTTGAAGCTGTATCCTCAATTCAGTTTAACATTTACCAAACAC
TTCTCCGTGGCACACAAAGGTTGGGACTTTTAAGATGAGTAAGACTCGTTGCTCATCAGAGAGGCAGACTTCTAGTCTAATACTAATATATTAA
TACTAATATATTAGGGTCTGTGCTGGAGATATGAACAGAATACTTAGAATAGTAGGGAGAACAGCTCATTTTATTTGGTAGGGGAATAACAGAG
ACTACATAGGTAGGAAAAAGAAGAACAATTTCTTTTCTTTTTCTTTTTCTCTTTTTTTTTTTTTTTTTTTTGAGATGGAGTCTTGCTCTGTTGC
CCAGGCTGGAGTGCAGTGACATGATCTCCGTTCACTGCAACCTCCACCTCCTGGGTTCAAGCGATTCTCCTGCCTTAGCCTCCAACTAGCTGGG
ATTACAGGCATGCAACACCACACCCAGCTAATTTTTGTATTTTTAGTAGAGACAGGGTTTCACCATGTCAGCCAGGCTGGTCTCGAACTCCTGA
CCTCGTGATCTGCCTGCCTTGGCCTCCCAAAGTGCTGGGATTACAGGTATGAGCCACCACGCCCCACCAGGAAGAACACTTTCAAGGTGACAGG
GATTGGCAGAAGCAGAGGGGGAAGAATGAATCATGTTTGTGGTGTACCTCCTGGAGGAGGGTCATAGAAGCTGAGGATATAAATTGGGACCAAA
CTGATGAACCCTAGAAACCATTCTTAATTCCAAAAGCTAAGGAACTTGGACTTTATTCTGTAGACAGTGGTTTTGAACCTTTTGAGAATGTATG
TAACTATATATAACTGGAGTGTGTGTGTCTGTGTGTGTTATAATATACATGCATTTGTACTGCTACAAACAGAAGAATCTGCATATTGTATTAT
ATGATATACAGTTATATATAGTTACATACACATATATATATATGCACACACATATATACACATACACTTATATACACAGATATATATACACACA
CAGTTATATACACACGCAGATATTATATATTCTAATTTAAATATGTAATCTATATACATCGAGTTTGTAGTACTGTTGTAGGCCATAAGAAGTT
GTTGATTTCCAATAGGGAGCGGTTAGAGGCAGGAACACCAACAGGTATGCTGTTTTCAAATGAAATGAGTTACCGCAGTTGCACTGGAAATACA
GATGAGGGAATGTACTTCAGGGGTTTCAAATAGGGCATAGGGACCAAGATGAACAGGAGATGTGAAAAGGAGAAGAGTTTAAAATGGTCTTGTT
TGTATGGCTGTCATTAAATTAGATAAAGAACACAGCAAGAACATTCACTTTTGAGTGAGAAGGGGAGAGGATCACTTTGGTTTCAAACAAGTTA
AACTTAGTATTACCGCCGTACATCTTAGTGGACATGTTGGTTGACAGTTGGAAATATAGATCTGAAGCTTAGAGAAAAGAGCTAGAGATGTAGA
TTTGAGAGTTATTAGCATTAAGTGATAGGTGAAGCCTGTGTTGGAATGAACGAAATTACCTGGGAGTGGATAAGGAGGGTTTCAGGGAAGAAGA
GACTTGAAGATAGATAAGGCATATCAGGAAGTAGAAAGAAACATGGGAGAGATTAATGTCAGAGAACATAAGGAAACCTTTTAATGTCAGCAAC
TAGAGAAAGGTGGAGTCAGATAAGGATTGATAGAGGCTATTGGTAGTTATTAATAACCTTTGAGAGAATGTATAACTTCGGCCATCATACCTTA
AGAAAAATGTTTAGTAATTTTCGTCTCTTTACATGTTATAATCTTAAGGTAATATTTCTTGAAAAGGCCTTTAAGGCCTTTTAAATAGCAGCAT
AAAGTTGAGGTTTATGATTTCCAGAAGTTGCCTTTGTTTTTTGTTTATTTTGAAAGTAAAAATCAACTTTAGGTAAAATAATTTCTAGTAAAAA
TCAGTTGTTTAGGGAGTGGTGTCAGAAAGCTGTAACTGTTGGTGTGATAACTTTTTCTTAAATGTTTATTCTTTCTAAGGGATAAAATTTATAC
ATTTAACAGAAAATTCTTTTTCCTTTTGCATATAGAGTTATTATTAAACACACTACATACCCTTTAAGGTTCCAAGTGGTTCATGGTTTTACTT
ATACATTTTTAACATTTTTCTAGTTCAGTATCATTTTCTTTTGTCATTAACACACAGGTTTTTTTTTTTTTTAATTTTGAGCTATTGCAAGTTG
AGTTTTCTTAATTATAATTTGGAGCCACTCAATTGTAGATTAAACACTGACACACCATTTTCTGGAAATGAAATTTGAGCTAGAATTTAGAGAA
TCATCTTTTAAACTTAAGATCTTAGATTCAGTACAAGTTGAGCATAGATAGATTTTAGGGGTTTTTTATTTTTAATTACATGAGTAATGTATGG
ATTTTTTTTTTGTCATTCATTTATTTCACATTTATTCTCATTGTACCACGCAAAGAGAGCAAGGAGTCATGCACCAACACACACAGATTGTGCT
GTTTTTCAGTCTTTCTGATAGAATCAGGATCCAGATTCTAACTGTCTTCTAAATCTGGATCAGTCAGTAGAGATGGACCACTGTGGTGGAGGGC
CCGGGGCTGCTGAGAGAAAGGCAGGATACATCTCTAGAACTGACCATGGCAGGTTCATGTTCCAGGGCTCCCATCTGGGTACCTGAGCATTTTC
CATATAGATCACAGGTGTTGGGGCCCACATCGGCTCCTGAAAGTCTCCGGGAATGCCATAGGCGTGGGGCTGGTGCTCGGGAGGTGCAAGGTCA
GCCACATCAGCACAGGGAGGACCTGAGAAGACGCTGATCTTCCATCTGTATCCCAGAATCCTGTGCAGGGCCTCTCCATAAGGACATTGCAAGG
GGTGGCACCTTGGCCTCTGGTTGGCCTCATGAATAGTGAAGTATAAGTCCTGCAAGCTTATCAGCATCTGGAGACATTCCTGCCAGCTCTTCTT
TATACCTCTGTGCTTGAGATGCGGGGCAACTGCTTTTGATACTGTGTGATACTTCTTCACCCCGTACACTTCACGCTCAAGAAATTCCCATTCT
TCCAGGAAACTCTGGACTTCCTGGCTACTCCATGGTTTAATTAACTGGACTGAGGGCTTTTCTGGTCCCTGGACTGCTTTTTTTCTGCTTCATT
TTCAGGATGTTTCTGGTAGTTTCAGTCTGAAGTATCACTTTCTCCATTTTTATTCCTGAGTTTTCTTCTGGGATGATTCTCACTGAGTCTGCAG
GGCCTGTTCCTCGGTTGGCGGAACACTGCCACTTACTCTGCTCTAATGGAACCTACGACAGTCAGAAGGAGCCTGAGTGTGGAAATGTGTTTGC
TTCTCTCTCCTTCTTATGTATGGATATTTTTATTTTTGTAAAAAAATTTTAAATTATGGCATAGATAATCCCATTTTTTTTTTTTTTGAGACGA
AGACTTGCCCTGTTGTCCAGGCTGGAGTGCGTTGGCATGATCTCGGCTCACTGCAACCTCCGCCTCCTGGTTTTAAGCAATTCTGCCTCAGCCT
CCTGAGTAGCTGGGATTACAAATACCCACCCCCACACCTGGCTAATTTTTATATTTTTAGTAGAGACGAGGTTTCGCCATTTGGCCAGGCTGGT
CTCAAACTCTTATCCACCTGCCTTAGCCTCCCAAAGTGCTGGAATTGCACGCGTGAGCCACCGTGCCCAGCCTAAATTACTTTTGACCACTGCC
TCCTCCCAGTCCTCTTCCCAGAGGTAATAATTGTTATTAATTCCGGGCATAATTGTTTATATTAGGCATCTTTATATGCTAGGAACTGTTAGTA
CAAAATGAGAGAAAAAGAAAAACTTTAAGCATATATTTTTATTTCCAATCAGGATTCTAATAATATTGAAATTAAAACATAAATGTTTTACCTC
CTATTATGTATTTTTTAGTTATTTTGTTATTCAAGAGCTCAAACAAGTAAACTATGACTATTTTTCTAGAGCTCAAACAAGTAAACTGACTATC
TTAGTAGACTCTGGCAGTTCTCCCCGACCCCTCACTCCACCTTGGTTCAGAACCATATAGAAATGAAAGCATTTTACTAGAATGTCAGAGACCT
GAATACCAGTCATGACTATGTCACTATCTATCTATGTGATCTTCATTGAGTCATTTTCTTAGGCCTGGTTCATCCATAAAATAAGATTGGGTTT
AAAATTGCTAAGGTCTTTTCCAACACTTTCAATTGTAATTTCTGCTCACATTTAGCTTTTAAGCAAAATTTACTGTCTATAAATTGCTTATGGC
TACACTTTGGTGTCTGTTTGCACAGCCACTATATCAGTTATCTACTCCTATGTAAGTACTCCAATACTTACTGGCTTAATACAGCAACCGTACA
TTTACTCATGATCCTATTGTTTGTCAGTTTGAGCTGAACCTAGCTGAGTGGCTCGGCTGGTCTCGCCTGGGGCTGATGTGCAGTTATCTGGGAG
ATTGGGGCTGGCTGGTCTGATTCATATGTCTGATGGTTGGTGCTGACTATTAGCTGGACCTTCCTCACCACATCGTCTCCTGATTGGTTCTAAG
AAGGCAAGCCCTAATGCAAAGGTGTGTCACATTTGCTGATGTCCCATTGGTAAAAGCAAGTCACATAATCCAAGAGTCAGTGCTATAGGAGACC
ACACAATGGTGTAGATAACCAGGAGTTATGGAGGCCATTAATATACCAGTTTACCACAAAGACCAAAATATGTCTATCGAAACCTCGTACACTA
AGGCTTCGAAGTGCTGCCATTGTCTCTAAATTGCTGCTGTTGACTGTTTGCCAGGATTTGTTCAGTCCTCTGGTCCTGTTTCATTTTTCTATAG
ACAGTAATATTTGTCACTGTTTTTCTTTGTTTGTTTTTTTGAGATGGACTCTTGCTCTGTCGCCCAGGCTGCAGTGCAGTGGCGTGGCATGATT
TCATCTTACTGCAACCTCTGCCTCCTGGGTTCAAGCAATTCTCTTGTCTCAGCCTCCCGAGTAGCTGGGACTACAGGCGCACACTGCCACGCTC
GGCTAATTTTTTGTATTTTAGTAGAGATGGGGTTTCACCGTGTTGCCCAGGCTAGTCTCAAACTCCTCAGCTCACGCAATCCACCCACCTCAGC
CTCCCAAAGTGCTAGGATTACACGCGTGAGCCACCATGCCCGCCCTGTCACTGTTTTTATCTCCTCTTTTAGCCATTTCTGCTAGTGCTCAAGT
AGGAACAGGACTATCAGCATTTTCCTAATTGGTGAATTGTATCAGCCTCAAATTTCTAAGAATATATTCTATCTAAAAATGGGTCCCAAGGGTA
ACACAGTATTTACTGATGGCAAGAAAATGCTGCTGAAAAATGGTATTACAAAGCTAAAAATGCTTTTTTTTTATTTAAAGATCAACAAAGGTGG
TTTCTGTGCCCACAGTTCCTTCATTTCCTTTTGTGTTTCCTTTCTTTGTCTGATTTTAAGGAACTAATACAGCATCTGGTGTGTATATGTTTGG
TTTTAAGCTCTTTCTGTCTGTGAGTTTGTCAGGACACTGAGGAAATGAAGTACTAACTTTAAATGATTTGTTGGGACGTTTGTTTCTTGTTGAC
TTTTTTTTTTTAAGAGTTGGGGGTCTCACTCTGTTGCCCAGGCTGGACTTGAACTCCTGGGCTCAAGCAGTCCTCCTGTCTCAGCTTCCCAGGT
ACCTGGAACTACAGGCATGCACCACTGCACCCAGATGTGTTTGATTTTTTTTGAAGTTGGTAACTTCATAGCTAGTGAAATAATCAATTAATTT
TGTTTAAAATAGTAATCAACAACTGGCCACAGTGGCTCATGTCTATAACCCCAGCACTTTGGGAGGCCAAGGCAGTTGGACTCCTTAAGCTCAA
AAGTTCAAGACCAGCCTCGGCAACATGGTGAAAACCCATCTCTACAAAAACTATAAAAAGTAGCCAACTGGGACCAACTGTAAAAATTACTTCT
GGCTACTTGGGAGGCTGAAATGGGAGGATCACCTGAGCCCAGGAGATCGAGGCTGCAGTGAGCTGTAATCATGCCACTACACTCCATCCTGGGC
AGCAGAGCAAGACTCTGTCTCAAAAAAAAAATTAAAAAAAATACATAAATAAAAGAAAATAGTAAACACTAAACTAAAACAATCTTTGGGACTA
TTAGAGGCACAAAAAGGGTTTTTAAGATCATTAAGTTAAAAGTCATTCTAATGCAGAGGAGGCACATCAGATGTAGAGTTTGAGTAATTCACCC
AAGATTACACAGCTAATAAGTAACAGAATTGACACTAGGTTATCGAATTAGTGTTTGTGTTCGAACGAGCACAAACAGGCATGCCTTGGTTTCA
AAATCTGGCTGCAGCACCTGTTAGCGGCAAGTTTAAAAATATTGTTCAGCTTTCCTCATCTGTAAGATGGTGATTATACCCTCTACCTCCAAGA
TTGTTAGGAAAATTAAACAAGAAAATATATAAGAATGTCTCCTACCTGGTAGAGACTCAGTAAATGCTCGCTTTTCTTTCAGTTCAGTGCTCTT
TCCACTGTGTCGGGCTGCCTACCTTTTACTGTTAATGCCTCCCAGTTTAGTGATTAAACAACAAATCAGAATCGTCAGGCTTTTTTTTTTATTC
TATTTGTGTTTCAGAAACTTGTTCCTTCTTTCCCCATCCTCTCTTCTCCCTCTCCCGTACTTCCTTCTCATTTATCCTTCCACCTTTTTTCCTC
CTTCATTCTTTGTCTGTCTTCTCCTCTCCCTCTTCCCTGTTTTATTCCCTTGGGCTGATCATGTATATTTCTTTTTATATGTTATATTGCCTAA
TAAAGTGCTGGACATCTTGTAGGTATCCTGCTACCTATTAACTTCAAATATTTTTCTTAATTTTCCTGTGCCATTCTTATATATCATTTTCTCT
AACTTTGTGGAATCATGTACTCTTTCTTCTCTGTTTCTTCAGGCCAACTGGCCTCATGCAACTAGATCTTTACATATCAGAAACTGACATCTTA
AAAAGGAGCTTAAGAATAAGAGCTGGCAATAAAATTACAGATTATTCTCAGCTCACAAATGACTTGTTTATGCAGCTAGTCAGAGAAGGGTTTC
CCTCTCATAGGACAACAAACATTCAACTCTTCAGTCACAGTGAATGTCATCATACTCATGATGCACAATAGGAGGAAGACGCTGCTTGCTTTAG
AATATATTACCTGTGTGACTAAGAGAGGATTCTCTTTTGGGAAGGCACCTGTGACAGATGAGATGTATTTCCAGTGATCTGAGAGTAGTGAGTC
ATAAATAGGACAGGTAAATTTCTACTCTAGTTTTTAGCAGCATTGTTAGTTAAATAGAGTTTGGTTAGGCAAGCCTGGAAATCAAATCAGCATT
TACAACACAATGCCATTGTGATTGTTGAATGAGGGAATGAATTTGTCAAACCAATAAACTCCTAAATAAACTTTGTAGGCAACCCTCTTTTAGA
ATTTGGGACTTGTTTTAGTGGTGTTTGTATGTATTATATGTATGTACATATGTACATCTATATACATATTCATAAATTGTGTACAAAACTAAAA
ATTATGGAAATTGTAAAGTAGAAATTTCACCAGAAGTCTACTGATTCGTTTCTGGATGGTAAGTAAACTCTCTTAAAATTTTGTGCCAGATATT
ATCTGTCCTTTTTCTGGGGAGAGGACATAAAAGATACACAGTTACAAAGATTCTTAAAGGGAGGGTAATGGTTCTCAAAAGGTAAGAGCTTCAG
CTATAGCATGTATAGATATATTTCTGTATGTACATATATATTTTTTGAGTATCAAACTGTTATCCATTGCCTTAACATTTTATTTATGTCTGGT
GTGGAATCTGTGGTAATTAGGACTACTTGATAGAATTATGGTAATCATTTGTGGTGTGCAATATTGAATACTTTGTCATAATTTTATATCTTAA
GCCATTCCTGGTAATATATCGATTTGCCAGGAATTTGATACAATTCATAGATAAAACATATCCAGAGATTTGGATGATTTGTCTCTTAACCTAA
ATTCTTCCTGCAGTTTAAGGAACATTTGAGTATTCCAGTTAGTACCTGAATTTTATATTTGGCTTTTAATCTGTGTATTTAATACTATAAGAAT
TTCTGTTTTCCCATCATTTTTATCTTAAATTTAACCAAAAAAGAACTAAAATACATTATTTCCCTAGTGTCATTTTCCATGTGATTTGAATGAA
CTATAAATGCTGTATATTCACATAAAGATTTTGATCTTCAATTACATTTTATCCAAAGTATTCAAAAGAAAGTTGGGAAGATACCTATTTGTTA
TCAGGACCAATTTATTTTAAAATTTTATATGTTTTAACTTCATGTATTCATAGACATATAAAGATGAACTTTCAATACGCCTCCTTCGATTGTA
TTTTTTTAATACAGTTACCACTCGTATTTTTTTAATACAGTTACAAACTCTTTAATAAAATACAGCTGTGTACTTTACTATTTTCTTTAAAATT
AGATGTATCGATTGTCTTTACAGTTTGAATTCCAAGACATGAATGAGAAGCTTATCAAAAACTGTAATGTGGTTACATTCTAAAAATAACAGTT
TTATTGAGATGTAATTCACATACCATTAATTCACCCTTTTAAGGTATACATTATATTTGCTTTTCAGTGGTTCAGAAAAAGAATCATTATATAC
GTTAGAGGGAGAGAAACATTATAAAAGTTAACAGTAGGGGAATGTGAGTAGAAAATATGGAAATTTTTAGTGTACGGTTCTTGTAACTTTTCTC
TGTTTGAAATTATTTCAAAATACATCTTTTTTTCTTTTTTCCAGCACTAATGGCTTGGGTGAATTAAAATATTTTTTAAATTAAAAAAAGTGTA
TGGTGTGGTGGGGGCCTTAATATATTCACAGAGTTGTGTAACTGTCACCACTAATTCCAGAGTGAAATTTTAAGCATAGTTTGTGTTTGTTTTC
CTGTTCCTTCCTTTTCACTTGATATACAAAAGTTGACGTATCTTGACAGAAAAATTTCTTTACAATTTTCTTAAAGTTTTCAAATGTTGAACTA
AAAGACCAAGGTATATCATAAGTACAAGGAAATAAAGTAGAATTAGGGCGTATAAAATGAAAATTCTTACTTGACAAAAGAAAGTGAACTGCTT
TTAACTTTATATATCCTTTGTTGAAACTTTTGTGTAGAATCCTGGAATAAATCATTAGTCTTATGCAAAACTGTGTCTACTTCTTAAAACTTGA
ATTGAGGAAGGAAATGCTTTGTACCAACATCGCAAATATTTTATTATAAGAACATTTCTGTTTCTTCCTCTCATACTACTATGTTCATTCTACT
GTATCTTCCTCTGAGAGGGTTTAAACTCTTTAGAGAACTGCCTTAATTTTTGTATGTATTGGAGCAACATAATCAGTCTTTGCAATGTTTTGAT
AAAAGTAAATTCTTAATAATAAAATTGTTATAATCTCCCATAATTTTCAAGTCAGTTTTCACTGTTGTTAATGATGTATGATATTGTTTTTCCT
TTTCTCTTAATTGGAATTTATAAACTCTTACTAAATGCAGATACATCTGCTCAAAACTTGTCATTTGTTCTTAGGGTCAAAAAATTGGGAAGAT
TAGTTTGCACTACCATTGTACCTTGCTTTTTATAATGAGTGTACTCTGGGTAAAAACATGAATCTCAATAAGTACTAAAAAAATGCAGTTGCCT
GTAATACAACTGAGGTTTTATCTAATCCCCTGATTCAAAATGACCCAGACAAATGTCTGCCAAGGGGCAAGGAGAAATGAGACACTTTTTCATT
CTTTAGAATGTAGAAAAAATGCTTTATAAAAATGGATTTTTATGTCAATAGGTACAAATCATTTTAAAATATTTTCTATAAAGTATTATATAAA
ACAGTGCTAATGAGTCTTAGGATTATACGAAGCATGTTTTCTTATATTGTTTTACTTATTTTCTGTAGGAAAAGTAGCCATCAAAAAAGAAGAC
TTGTGTAATCACAGTCGCAAAGAAACTTGGTTTTCATTACAGCCTGTTGACTCCAATTCAGAGGTTCAGGTAAATATTAAGGCTTATGTAATAC
AAGAGATTGTCACAGGAAATGAGTAAAAAATGAACTAATTAAACTGGTTCATGTAAATAGAGCATATTATACCTTATTTTAAATTCTAGCATCA
AATATTATAGACTTTTGGGTAATATAGGACAGCAAGGTAATGGCTTTGTCATATTGTTTCAAGAATAATGCTCATGTATATGCATATGTGTAGA
TTTGTGGAGATTAGGGCATTTCTTTCTTACCTTCTGTTTGAGTCACAATAGCAAATTATTATTTGTCTTTGAGGGGAAAAGTTTTAAGGGAAAA
TATGTGTTTTATTAACTCTGACTTTGGGGGAGAATCCCTGGAAGTCTGATTTTGTCTGTCTTGATTCTCTGTTTACTCTCTAGTAAACTGGACT
TGGGGAAATGTTATTCATTTAAACCCTGCCTGAGCCCACTGTGCACAGCCTTCACTCCCACACCCACCCTTTCCTGTTGTTCACCTCGCTTTTT
ATTTGAAAGAGTATCATTACTCTTTAATCTTTTTCTTTTTTCCAAAATAAGAGACCTTTTTCAGATTCTGAGTAAAGATGTGTGTTTAGCCATG
TAGCTGAAGTTTCTATTTGTTTTTAGAAAATCTGAGTTGCTCTTAGAAATTATTTTTCTAACTAAAGGGAATCTTTTTGCTGTAGCCTTATTTA
TTTATAAAATATGGTAAGGATTGTGAAACCATTTCCCGAGGGCATAATTGCAAGCAGGATTCTTCCTCTCTAAAAGAAGTTTTAATTTGCCACT
GTGACTCAGAACTTTAAGACAAGAAAAAAGTGCTACACCCACACCACAGATAATTTGCAAATAAGTGCCTGTGTCTCCATTTCCTCATAATCTC
TCAAGCATTCTCTTTGATTAATTATTAAATTCTCAAAAAATTAACTACTTATGCACTAACCCCCCTTCTTAACTGCAAAATTTAACATACTTTT
CCTGTTTAACCTACTTTGCCATGTTTTAATATTCTTGTCTTTCCTTATGACCATAGCTATTATTCAGATCCCAGTTGTACAGTATTGTAAATGA
TCCATGTGACAAGCAATGTGAAGAAGTGGAAACTGTTTAAATTAAAAGGCAGAAAACTTAGGTCCTAGTTTTTATATGTAGTTACTTAAATGAC
ATTGAATTTCATTTCTATAAAGTGGGAATAAAAGTTGCTGTCTTCGATTCATATGGTTGTGAGAATTAAGTGAAATATCTGTAAGAATAGTTTA
AAAGCTGTTAATGCTATACAGATGTAAAATATTGTCTGTTTACCACCAGTCAGTCTGGTCGCTATTTCCCTAATATTCCTTTACCAAAAGGCAG
ATATGAAAGTTTTAAAGTACCAGTTTTTCTTTTAATAGTTTTAACAAAACCCATGATAGGAAAAGGCTTTGAAAATTTAAACTGAAATTTTATG
AACCATTACATTTTATAAAACTCATAAATTTTGTGAATTTTATGTAAATTTGCTAAACAATTTAAGAGTTCCAGCTCTCAAAAGGTAAAAATCT
ATATAGAATATAGAATTCTTGTAGATGGTACTAAAGATTAATACAGAGACACAAGACTTTATTTGATGGTGTGTGTGTGTATATATATGTGTGT
GTATATATATATGCATATGTGTATATATGCATATGTGTCTATATATATGCATATATATGTATATATATACACACACATTACATACACATGGGTA
TGCATATTTATAGATATACATATATATGCATATATGCATGTATATATACACATTTGAGAGATATATATCTCATCTATGAAACATGAAAATTGGG
GAAGCAACAGTTACCCAGATATAATATAGTCATTTAAAAATTTTGTACAAGTTATTTTCATGACTGTGAGTTATTAAACTTTACAACTCTTTTG
TCTTAAATGATACTTACTTAATAAGATTTGCTCACTATTTAAAACACATTTTAGAAGCAAAAGTACTTTCTCGGGTGGAAGAGACATATAACAC
TTTTGGGTTAATATGGTACCATTGAAAATGTTCTTGTCCCACTGCTGATAAGGGAATACTAAATTATGAGTCTAGAAAAGATAGAAAATCATCC
CATGGTTCATACAAATCCTACTTTGTTAATAAATTTGTTCACTTCCATAAACTTTATAAAATTTTACAAAAATACCATGAAACATAAGAGCAAT
TAATAGGGAAATAGGTGAAAGCTATGAAACAGATTGCGTTCAACAGGAACTCAGTCTGATAATTACTATTAGTAATTATACCAGTGTATCCTAA
GCACAGTACTAAGTACTTAATGTCATTTAACTTTCAAAGTAGCATTATGAGGTAAATAGCATCACCTGCATTTTACAAATAAGGCAAATGAAAT
TCAGAGGTTAAATGTCTTCTCCAGAGTCACAGAACTACTAAGTAGTGGAGCCTTGATTCCAAAAGAGATCTGTCTGATTCTAAAACTCCTAACC
ACAGTGCCTTTACTACCTTTCACCATTCTGTACTATTAGTCATTAAAAAAAATAGATTAGGAGTATCACTTAAGTAACATCTGAGTGTAAGGAA
GAGAAGCCTGCTCAGATTTGCTCAAAGAAAATGGGAGAGGCCATTGGAAAGCTCCTAAAGAGTTTCTTCGAATGTCATCATAGGAACTTTACAT
TGCCAGTGTGAGCTAGAAACTTCTCAGGCATTTGCAGAGGAGATCCATCTTAAGTGGAGGCTAGATTCTTAGAACAACAGACATTAAAATTTGC
CTAAGTTCATCTGTAAAATGAGTATACAAAATGAAAAGTTAAAAATTTTTGCGTAAGTTACAATTATTTTTGAAAAGAGTTTAGTTTCTTATAA
AATACACTAAGGTATTTGGGTGCTTTCTTTGTTTTTGGTTGACTACCGCATAAAATAATTGGCTTGCATTCTGCCCCATGGTAAATGAAAACTA
TATACTGTATCTTTAAACTGTAGACCTCCCCCCTGCCCTGACTCAGTACAGCAATAGATACTGCAAGTGGCACATGGAAACTGAGACCCACTAA
TCATTTCTTATATTCCTATGTTCACTGTGCTTATTGAGCATAAGTTCTCACTGTTTAAATATTGTTTTTTTTCTTTCCCTTTCATGAAGTGAAT
AATGTTTTCTCTCAGTCTTTCCCTCTCATTTTAATAACTATTTGCTTTGCAAAATATAAAGGAAACAAAAAAACACCATTTTGAGATGCCATTG
TATTCTTTTTGTATGTATGATGAGACATGTCTCTTCCTCTGCTTCTCTGTAAATCTACTCCAGTCTCTTTTTGAGACCTAAAAGACGCCACACC
AATTTTTGAGTCCATGTGACCATACAGCTTCCTCACCTCCAACATCTCACTCAGTTTTCTGCCACAATCTAGAGAGAAAGTTTCGCTAAACTTG
GGTCAGATGTCTACTTCAATCTAATTTGATATCGTTGGAGGAAAATTCACTGTAAGCCAGTCCACTTTAGCGCTTTGAGTAGGCATGTTCCCTA
AGGAAGGGGTTTGAGTAGAGGAAATGAATGGCATTGCTACCTCAGAGGGAGAGATGCTTCTTTCCTAGTTCTACCACTAATTAGCATTTGTGCC
CCAAATAATCGCAAGCCTGTTACCTGAGGATCTTCATCTATCAAATGATAATGTCAGCGCAAAGGGTTCCCTTAGTTGTGAAGTTCTAGGGAAA
TAACATGCAGAAATGTAATTTAGCTTTCCAGTGGAAGGATCCCAGTAGTATTAAGATGGGCTTTATCCCAAAACAGTTGAAAAATTCTTTAGAA
CTATAACCTCTTACCTTGAATTGTCTCCCAGAGATAATTTTTTATTATTGCTGATACTGTTATACTTGTCAATAATGTTAATAGTTCTTTTCAG
TTTCATCTTCAGTAGTTCAAAATATTGTTAGTAACATTTCATGAGTGTATTTTTCTACTTTTTTTTTGTATTTAGATTTTGGCTTGATTCTACC
TTTTGTAATTTTTCCTTCTGGGCTGAACCATTTTTTTCCTCAACCTATTCTAATCTGCTTTCAGACTCTGCAGTGGGTCAGAGAGAGAGCAGAA
ATGGTTCTTTTTACCAATGACATTGTGTCAATAAGTCTAGTGGACATTTTTCAGTCCTCATCTTACTTGATCTTTGAGCAGCTTTTGATACTGG
TGACCACTCCCTCCATTTTGAAGCATTCTTTTCCTTAGTTTCTGATCCTGTTTATGGTCCTAGTTATCATCTATTTCATTTGGTATTCTTTCTC
AATATTCTTTGCCACATATGTGAAGTTAATATGGGTTGTGCTCGTGCCTTCTATAAATCCTCTTTTGGCTTCAGTGAACTACAGCCGTGCTATT
AGTTATTAGGTCCTGTAACTCACCAGGAACATTAGGTATTTATCCTTTCTTTATTTCTTACGCACAGTGATAAAAAGCATACTTTACATATGGT
TGATTAACCAGGCCGGCTATCGTCGCCCACTGTAGTCCCAGCACTTTGAAAGGTTGAGGCAGACAGATTGCTTGAGCCCAGAAGTTTGAGACCA
GCTTGGGCAACATAGCAAGACCCCATCTTTATAGGGGGGGGAAAAAGAGTGATTAAACCAAATCTCCCATATGGAGAATTTGCTGCTCTCATTA
ATTAAGGAGTTTGTCTCACACAACTTGTCCAAGCAGTAAATCCAGATGTGCTATCAACCATGTCCTATATCATCCAATTACTTAGTTAATAGGT
CTTTACCAACAAGCATATCTTGAATTTCTGGATCATGCCATAAGTTATTGATTGTTTTATAGGTGTTTTTTTTTTAAAGACCTCTGCCTTACCT
GTTTAAAAAGACATTTAAAAAGAAATGCAATGATTATAGCTAATAATAAATACATGAGGATTTTTAAAACATGCCCTATTCAAGACATAACAAA
TGTAAAAACATAATTCATTATTGCAGGTTGATTTTTTTTAACTTCATTTTAAATTTTTATGTGAATTAATAATGAGCCATTCTAGATCATATTA
AATACTTCTTTAAGGAGAAAACAACACCTTCATAAAGAACTGCCATTAATATAAGAGATATTAAATAAAATTTGTATTTTGCTTTTTCATCTGA
TGGGCGATTGTGTCCAAAATCATTTATAGAGGTAATTTCTACGTATTAAAACATCCATCACAAACTCCGAAGTTGGGATATTTTTCACCTCATC
TACAGGCATACCTTCTTTCATTGTACTTCACTGTGTCGTGCTTCACAGATACTGCATTTTTTACAAATTGCAGGTTTGTGACAACCCTGTGTCA
AGCAAATCTATCTGCCTCATTTTTCCGACATCATATGCTCACCTCATTTCTCTATGACAGCCTTTTTTAGCAATAACATATTTTTAAATTAAAA
TATGTATATTGTTATTTAAGACATATTGCACACTTAATAGACTGCAATATACTATAAATGTAACTTATATACACTAGAAAACCAAAAAATTTTT
GTGACTTTCTGTTTAGATATTTGCTTTATTGAAGTGGTCTGGACCCTTAGACAGTATTTCCAAGGTATGCCTGTAAAAACATTTTTTACCAGTT
GAAACATACTGAGTTATTTTATATGGAAATGGTTGTTAAAATTTAGTGTTTTGTGATAAAAACAAAATAAAGTTTAGACTTCAGAGGATTTTAA
GTGCATGTATTATCTTTTGCTCAAATACCTCTATATTAGTCCATTTTTGTGTTGCTATAAAGGAACATCTAAGGCTGAGTAATTTATAAAGGAA
AGAGGTTTATTTTGGCTTACAGTTCTTCTGCAGGCTGTACAGGAAGTATGGTGCTGGCATCTGCTTCTGGTGAGTGCTTCAGGAAGCTTACAAT
CATGGTGGAAGGTAAACGGGGAGCAGGCATATTACATCATGAGAGAGGGAGCAACAGACAGAAGGCAGAAGTCCCAGACTCTTAAACAACTAGA
TCTCACATGAACTAACTAAGCAAGACGCACTTATCACCAAGGGGGTGGCACTCAGCCATTCATGAGGGATCTACCCCCATGATCTGACACCTCC
CACTAGGCCCCACCTCCAACACTGGGGATCACATTTCATCATGAGATTTGGAGGGACAAACATTCAAGCCATATCAACCTCTGTTCATCTTATT
TTCCCAAACTGTATGCAGAAATTTACACAGTATCTTCTATATGGTACCAAGAATGTATGTAATACACAATTCAAATAATCACAGATATTTTTGT
GTGAAATTTTTGTAAAATTAATTGAAATAAAGTTAGCGTATAAAATACTAAATCTTCAAATAGTACCTCTATTAGCCCATTTTCACGCTGCTGA
TAAAGACATATCCAAGACTGGAAAGAAAAAGAGGTTTAATTGGACTTATAGTTCCACATGACTGGGGAGGCGTCAGAATCATGGCAGGAGGTGA
AAGGCACTTCTTACATGCCAGTGGCAAAAGAAAATGAGAGAGAAGCAAAAGCGGAAACCCCTGATAAACCCATCAGATCTTGTGAGACTTATTC
ACTATTACGAGAATAGCACAGCAAAGACTGGCCCCCATGATTCAATCACCTCCCCCCGGGTTCCTCCCACAACACATGGGAATTCTGGGAGATA
CAATTCAAGTTGACATTTGGTGGGGACACAGCCAAACCATATCAGTACGAGAGTAGTGTAAGTTCCTCAGAATGCCCCTTAATATAACATAATG
TGATATTATAACTGCATGTATCAGCCTAACATAGTCAGTGCTATGGAAGTCATATAGAGCAATACCTTCCAAGAATTTGGTTTATTTTTTATTA
AAGACGTATAATACACTGCAAGGTATCTGATCTTCCAACATTTTCTGACATACATGAAAAGTATACTGTCATTGAGCTTCCTTTAGGAGATTGA
ATAATAAGAATGCAGTTGTAGACCAAATCACATTACATGTTTATTATCAAACATAGTACCTTGTTAGATTCTTTTTTTTTTTTTTTTTTGAGAT
GGAGTTCTGCTGGAGTGCAATGGTGTGATCTCAGTTCACCACAACCTCTGCCTCCTGGGTTCAAGTGATTCTCCTGCCTCAGCCTCCCGAATAG
CTGGGATTACAGGCATGCGCTGCCACACCCGGCTAATTTTGTATTTTTAGTAGAGACGGGGTTTCTCCGTGTTCGTCAGGCTGGTCTCAAACTG
CTGACCTCAGGTGATCTGCCTGCCTTGGCCTCCCAAAGTGCTGGGATTACAGGCATGAGCCACTGCGCCCAGCCTCCTTGTTGGATTCTTAATG
TTACTTCTTCGTAGGTTCTTTATTTTTCTTATTTGTCCCTAGGAGTTCTTGAATCTGTGGCTTGTCTTCTATCAGTTTTGGAAAATTCCAGGCA
ATTATCTTATCTCCTTTTTTTTTCTGCCCTGTTCTTTTTTTCTCTTCTCTGCTGTGACTCCACTTACATCAATAATAGGCCATTTCACCATGTC
TCATATGTCTCCTATCCTTTTTCCCTCTCTGTGCTTCATTGTGGATATTTTCTACTGACCTGTCTCCCATTTCACCAGTCCTCTTTTCAGCTGT
GGCTACTCTGCTACTAAATACATTTTCATTTTGGTTTATTGCTTAGTTCTAACTTTTTTATTTCATTCATTTTATAGTTTCCAGGTATCTGCTG
GATTTTTCATTTTGTTTTATGTCTTTTAAAAAATAGCTATTTTAGGCTGGATGCAGTGGCTCACACCTGTAATCCCAGCACTTTGGGAGGCCAA
GGCGGGAAAATTACTTGAGGTCAGAAGTTCAAGACCAGCCTGGCCAACATGGTGAAACCGTGTCTCTACTAAAAATACAAAAATTAGCCGGGCG
TGGTGGCACATCCCTCTACTCCCAGCTACTCGGGAGGCTGAGGCAGGAGAATCGCTTGAACCCGGGAGGCAGAGGTTGCAGTGAGCTGAGATCA
CGCCACTGCACTCTGGCCTGCGTGACAGAGCTAGACTCTGTCTCAAAGAAAAAAAAAGCAATTTTAAATTCTGTCAATTCCAATATCTGCGTCT
CTTGTAAGTCCACTGCTTTCCTCATTTTTTCTCTTGGTTTTTGATCTTCTGGCATATTCTGTAATTTTTAATCCAATGCTGGAGAATAAAATAT
AAAATATATTGGAGATAATTTGAGGGCCTGAATGGTATATTTCCTCTAGAGAAGAAGTATGATTCCCTGCCAGCAACTGCTGTTTCTATCTTGT
TGGCCAGAACTACATCATATGGCTATCCCTGATCTCAAGGGAGGCTACAGAATGTCAGTTTGCTGGGCTCATAACTGCCCCATCAAAAAACAAA
ACACACACACACACACACACACACACACACAAACTTGTTTTGCTGCAATTCAAATCTATTAAATGATTACAGATATAAGTAAAACTGAACTTTT
GTGTGAAAAGAGACTAAAAACTATTCTAAAATGTTCACTGATTTGAGTTTCTAAAACTGAAAAGCAGTTTTAAATAATTTAAAACTTTATTTTT
CTTTACTAAGAACAACACAGTAGTAGTAAAAACTTTCAAAATTAAATTGCTAGTATATACTTTTAAAATCTGAAAATTAAATTGGTAGTATGAA
CTTTTAAAATCTAGAGCAAAAAAGCAAAGAAAATCTTCTGAAATTTCACCAACCAAAGGTGTCTTCTATCAACATTTTGGTAGATGATCTTGTA
ATTTTATACATATATGTTTTTTATTTTTATAAGTTAACATTATAGTATGTACCCTATTTTGTGATTTATTTCTTTTGCTAAGGATAGTATGTAT
TTCCTTGTTAAAGTATATAATGAGTTAGTGTTTTACAGTTTAGAAATTATAATTTCCTTAATTTAACTCATAATGCCTTTTAATTTAGTGTTCC
ACCCTAACAGTATGTGTTTTGTGTCTGCCTGTTCTGTCCTCACACTTTTCTTGACCTGGTTAATATATAATTGTGTTATTGTTGATTAATACCT
AAGGAATGTTTTATTTACCAGTCAGTGGGAGTAATGAACATATTAGGTAGGCTCTGACTCAATAATAATGATTTCTGGCAGAATCTCATTAAAT
ATCCTTTTGACTTGCTATAATACTATGATTTTAGTTGTAAACAAAATGATTATCTTTTTCTACTCTATTGTCATAGTTTTTATTTCTTAGAACT
GGAATAGATCCCAGACATTTCCTGAGAGACAGTTGTGTTAGCATACGATTTCTGTAATAAAATGTATATATGTTAAAATTGTAAAACTGCAAAT
TTTTCTGCCGTTGGCTTGGCTCCCTTGCTAGCCAAGTAATACATACATGCTGTCACAACAGAAAATATGATTTCAAAATTCTAGTTGAGTAATA
TTACAAATTACAGATCATGGACCTTCAAGATCACCCTTGAATAGTAGACATTGTAAATACTAAATGCAGGAATAATGAAAGACCTAGTTTACAA
ATTATATATGATTTTATGAATATTTGTGCCTAGATTTTTAGTGGAAGATTTCCTTAATTTAGTTCCACCTCCACGTAACTGCTATTAATTTTTC
TACCACAGATGTGTTTGCTCCTTTAGACCTTAGCTTTCTCAGGCACACTTCTGTTTTAAGATCCATCAGTATTTTTTGCTCATCTCTTACTATT
CATCTAACCCTGAGCTTTCTACCTGGTGGACCTCAGATTCATTCTTGTATAAGTCCCCTTAAAATAGTGAGGGTGTTTCAGCTTAGGTTTCTGT
GAAAGAATTTTTGAAAAGGGTTGTTCTAAACAGCAACAGTGACTACATAATGAACTTTCCTACCTCTAATGAAATCACTCACGTCATCAGTCCC
TAGACATCACTCCCCTGAAATCTCCACATGGCAATGTGAGAATTTGTCTTGTCAGCTGGAGGGACGTAATCCAGTACTGTTTCTTAACCAGTTT
AGAATTGTTAGCGCAGAATTGCTCATCACCAGCGGCAGGTTAGAAATATCAAGCTCTACATTATATTGTTCCTAAGCCTTTATTTCTTGAGAAG
TTGATTGTTTGAATTCTTTTGAACTTACACTACTTAATTTTTAAAAATTCGTCTATTAATATCTCTTTAAGAAAGTACTCCTTTATTTTCAGTG
TCCCATCTTTCCATCAAGACTCTTGGCCATATGTTATCTTTTGTTTATTGTCTTCCCAACTAGATTGTACGCTGCTTGTAAGCAGTGACTTACA
TCTTTAACATCTATGGATTCCTACCCACTCCCACAGTGCCTTCTGAAACATAGTAGACATTCCATAAATCTTTATTGTTTGATCAAAGTAAGTA
GTCACAAATTGAGTTTTATTGCCTGTAACTTAGTTTTTAGTTGTTTTAGAATTGTATGATTTCTCTGTCTTTTAATCAGACTGCTTCTGTCCTT
AATGCTGGTTTATCTTCTGGAAAAAGCATCCTACATTTTATAATTTGGTTTTATCTCAAAATTTCCTTAAATTTACGTATTGTCTTTCCTTTTA
GTCTGTGATAATTAGATATATAAATATTTATTGAATTGGACATAAAATTCTTAAATTTGAATACCATGTTTGACATAAGTATTTGTTTCATATC
TTTCAGAACTTTGATCTCTGGTTTTGGTTATTATTCATTTAACAAGTACTTAGTGTAGGCAAAGCCATGTGCTAGTGGTCAGTGGATAAATACT
GTGGTGATTTGAAAAGGCACATACCTTTAATATTTTGTCAGTAAAATAGACTACTCAGTTTGTAATCTTTTTGTCATTATAGCGTAAAGTTCAC
CTTGAATTAAAACTGAATGAACTGATAACGGAGAATGGAACTGTATGCCAGCAGCTTGTTGTACAGTAAGCATTTTTTTTAACCAAAATCAACT
AGAAATAATTCTCCATTTTGTATTCTTTCGATTTTAAGCCAACTCCTTGTTTTAACTTCAGAAACTTTTTTCAAAAATGAAATTCTGCTATGTC
ATTCCTTTTGTGAAGTTGATTTTCAATTTAATTTTCTCATAGTCTACTCACGTTATGGTGAGTCATTAATTTATTCAACTAATATTTATTGAAT
ATGGGTTAAATGATATATATGGGCATTAAGGATAAGGGTGTCTAACTCTCTGGCAGTGGAGAATTGGAAAGGCTTCCCAAAGAAGGTCCTCTAG
AAATTGTCATACCACAGAGCAGAGGTTTAGAGACTTTGCAGCTAGAACTAATGCCCACTCAGAGATGACCAGTATTGATTCCCACCTACTTTGA
ATTCCTTTTTACCTCTTCCACCCTCAAGTCATTTTTTACCACCCAGACTCTAATCTCCTGGCTGTTCTGGAAATCTGTTGTAGCCCAATCCCCT
ACTCTTTTCCTCCCCTCCAAACCCTTACTCTGTGATATCAATAGACTCCCAGATAACCTTCACTTCTTCTGGGTGTTTTCCTCTATTTTTGCAT
TAGTTGACATCTGAATATCCCACAAAGTACAGCAGTTTCACAGCCTTCTCAGTTAGAGGCTTTGTTTTTCTATCATGTCCACATATTGTAAGGT
CAGGGAGTAGAGTAGATATTACTCTCCTATTGCTGCTCCAGACTGTAACTTATTCTCATCAATTGTGAACCCCAGCTCCTTTGAGGTATTCTAT
CTGTATATAAATATGTCTGTTTGCTACTATATATAATATTTATTTCATTAATTTTTTCTGACCTCTTTGAAAGAAGATTGCATATGTATGACTC
TTAATATATCAGTATGTATTTCCTAAGAATAAGGAATATTATCTTAAATAACTCCAGTACAGTTACCAAATTCAGGAAGTTTAACATTGTTACA
ATACTTTTTAAAAATCTATAGTTATTTTCTAATATTTTAATCTGCAGTTATTATATTCCACTTTTGTTAACTGTCCCAATGATGTCTATTAAGG
CGTTTTTTTCCTTCTAAGAAAGGATCTGCTCCAAGATTATGTTTTATGTTTAGTTGACATGTCTCTTTAATCTCCTTTAACATGGAATAGTTTT
TTGCTTTTCTTTGATTGTATTAACATTTTTTAAGAATATAAACCAGTTTTTTTTTTTTTAATGTTTCTCAATTTGGATTTACCTACTAAAAATA
GTTCAAGATCTGTTTGCAGCTTTTTCTCCACAGACTGACTCTGTAGACCAACTGAGAGCATATGGTGAAAGTATCTGGATTAGGTCTTCCACTC
CTCCCTCCCCTGCTTCAGTTTAATTATAATAGTCATTTGAAATATAGTTAGTTTGGTTGGTTTCGTTTGTTTGCATTCAATTTAAGGGTTTTTT
TGTGCCCCTTCCCCCCAATTTAAGTTTTTTCCCCCATCCTTCTAGGCTTAATTTTATTTTTCAATAGAATATTTGCATGATTTCAAAAATCAAA
ACTATAACAAAAGGCGTTACTCAGAGATGTGTGTAGTTCAGTTCCGTCTCCCCACACCCAGCTCCAAGCAACCATGATCTGATTTCTGTCCCTA
CAGTTTTTGCCTTTTCCAAAGTGTTATTTGAACAAAACTATATGATATGTCTTTTTTGTGTCTGCTGTCTTTCACTTAGCATAATGCTTTTGAG
ATTCATCCACACTGTACGTATATCATTTCCTTTTTGGTGTCTTATTTGTTTCTTGCCATAATTTGGTAGATACTGTTATCCTCATTTTGCAGAT
GACAAAACTGATGCCCATATTGGTTAAATAAGCCTAATTTCACACAGGAAGTGTTCGACGCTGGCAGTTTGATTTTTTTCCCCCAACTTTAAAA
TTTTGAAATAATTACAGATTTACAGAAAGTTGTAAGGATAGTACAGAGAGGTCCTGTGTACCCTTCATTCTTTTTCCCTCAGTGGGTACATCTT
GCATGACTATAGCACAATACCAAAACCAGGAAATTTACATTGGCATAATGTATGTATATAGTTATAGATCATTTTATCATGTGTGTTGATTTGT
GTAACTACCACCACCAAGATACGAAACTCTTCTGTCACCATAAAGATATCTCTCATCCCATAGCTTTATAGTCATAACCACTCCTCTCTTCTAA
CCCTTGGCAACCTCTAATCTGTCTTTATATCTATAATTTTGTCATTTCAAGAATGTTATGTGAATGGAATCTTATAATATGTGACCTTTTGAGA
TTGGCTTTTTTTACTCAGATCTATCTGAATGACTCTGTGTAGTTTTTTGTAGACAGAATATACTTGGGTCACTTTTTTAAAAAAATCATTTTTG
CCAATCTCTGTCTTTTTTTGGTGAGTTTTTTCCATTTATTTTTAATGTAATTATTGAATATTAGGCTTAAATATGCCATTTTTTGTTTGTTTTC
TGTTTGTCTCCTATTTTATAGTTTTCTGTTTTCTTTTTCCTGTGGGTTACTTGAACACTTTTTAGAATTCCATTTTGATTTATTTATATTGTGT
TTTTAAAAAATCTATGGTCTCTTTGTATAGATTTTTTTTTAGTGGTTGCTCTAGGTATTACATTATACATGCATAACTAATCATAATGTACTGA
TGTCAACATTTTACCAATTAGAATAAAATGTATGTACTTTACCTTTCTTTAAATCCCTTTACCCTCCCCCATTTGTAATATACTTTGTCTTAAA
TATTTTGTCTACATATATTGAGAACCACATCAAACAATGTTATAATTTTTGCTTCAGCCATCTAACACAGTTGAAAAGAGTTAGAGGAAAAGGA
AAGTGTAAGTCTGTTGTCTCTACCCACATTTTTGCCCTTTCCATGTTATTTTTTTCCTTTCTGATGTTTCACGATTCCTTCTGTGATTAAGTGT
ATAGAGTTTCCTCTTAACATTCTTTTAGAGTAAGTCTCTTGGTGATACATTCTCTTGGTTTTCCTTCATCTCAGAAGGTCTTGATTTCCTCTTC
ATTTGTAAAAGATAATTATACTAGATATAGAATTCTGAGTGCACAGTTCTTTTCTTTCCACATTTGAAAATGTACCACTTCCTTGTGTCCTCGA
TGGTTTCTCATGTGAAGTTCATTGTCATTCAAATTGTTTTTCCCTATAGGTAACACACCGTTTTTCTCTGGCTTCTTTTAAGGTTGTTCTTTGC
CTTTAGATTTCTTTTCTTTTTTCTTTCTTTCTTTCTTTTTTCTTTTTTTTTTTTTTTTTGATATGGAGTCTAGTTCTGTCACTCACGCTGGAGT
GCATTGGTGTGATCTCAGCTCACTGCAACCTCTGCCTCCCGGATTCAAGCGATTTTCATGCCTCAGCCTCCTAAGTAGCTGGAATTACAGGCAT
GCACCACCATGCCCACCTAATTTTTACATTTTTAGTAGAAACAGGGTTTCGCCATATTGGCCAGGCTGCTCTCGAACTCCTGGCCTGAAGTGAT
CCTTCTGCCTTGGCCTCCCAAAGTCCTGGGGTTATAGGTGTAAGCCACCACGCCCACCCTGCCTTTAGATTTCTATATGTTTTAGCTTGATTGT
CATGTATCCTGGAATAGATTTATTTAATTTATCCTGTTTGGGGTTCGCTCAGCTTCTTGACTATGTATATTTATGTCTTTTGCCACATTTGGGG
AATTTTCAACCATTATTTCTTCTATTAATTCTTTATCACCTCTGCTATCTTTCTCTTCTTCTTTCAGAACGCCAGTGGCACAAATGTTAGGTCT
TTTTTTTACAGTCCCAGAGGTCCCCCTGACGCTTTGTCCTTTGCTTTTCCCCCCAGTCTATTTTCCCTCTGTTGTTCAAATTGGGTGATTTCTA
TTGTTATGTCTTCAAGTTCACTGATTCTTTCCTCTGTCTTCTTCATTCTTCTGTTTACCTTATCTGTTGAGTTTTTTATTTTCGTTATTTATAT
TTTTAAGTTCTAAAATTTATATTTGATTCTTCTTTATAACTTTTTTCCTTTGGTGAGAGTTTCTTTTGCAGAGACTTTCTTGTTTTTTTGTTTC
AAACATATTCATAATTGTTCATTGCATTATTCACAATAGCAAAGACATGGAATCAACTTAAATGGCCATCAATCCCAGACTGGATAAAGAAAAT
GCAGTACATACATGCCATGGAATACTAGCCGTAAAAAAGAAAGAGATCATATCCTTTGCAGGAACATACGCGGAGCTCAACGCCATTATCCTTA
GCAGACTAACGCAGAAACAGAAAATGAAACCAAATACTGCACGTGCTCACTTATAGTGGGAGCTAAATGATCACAATGCATGGACACATAGAGA
AGAACAACAGACGTTAGGGCCTACCAGAGGGTGGAGAGTAGGAGGAGGGAGAGGATCAGGAACAATAACTAATGGGTGCTAGCCTTAATACCTA
AGTGACAAAATAATCTGTACAACAAACCCTTGTGACACAAGTTTGCCTATATAACAGACCTGCACATGTACCCCTGAACTTAAGTTAAAAAAAA
ATTGCTCATTGAATTATTTTTATGATGGCTGCTTTCAGATCCATGTCACATAATCATAACAATCATGTCATCTTGGTGTTAGTGTCTTGTGTTA
GTCCATTCTCACATTGCTATAAAGAAATACCTGAGACTGGGTAATTTATAAAGAAGTTTAATTCACTCATGGTTCTGCAGGCTGTACAGGAAGC
ATGGCAGCATCTGCTTCTGGGGAGGCCTCAGGAAGCTTCCAATCATAACAAACACCAAGCGGGAGCAGGTGTCCTACATGGCAGGAGCAGGAAC
AGGAGAGAGAGTGGGGAGGTACTACATACTTTTTTTTTTTTTTTTTTTTTTGAGATGGAGTCTCACTCTGTCCACCACCCTCGGGTGCAGTGAC
ATGACCTCCACCTCCTGCGTTCAAGTGATTCTCCTGCCTCAGCCTCCGAGTAGCTGGCATTACAGGTCTGCACCACCAGACCTGGCTAATTTTT
GTATTTTTAGTAGAGTTGGGGTTTCATCATGTTGGTCAGGCTCGTCTCCAACTCTTGACCTCGTGATGTGCCCACCTCAGCCTCCCAAAGTGCT
GGGATTACAGGCATGAGCCACCAAGCCTGGCCCTCTTTTAGTTATTTTTAAATGTATAATAAATTATTGTTGTCTGTAGTCACCTTCTTATGCT
GTTAAATACGAGATCTTATCCATTATATCTAACTATATTTTTGTACCCTCACTTCCCGTCCCAGCCCCTGGAAAACTTTGTTCTACTCTCTGTT
ATCATGAGTTCAATTGTTTGAGTTTTTAGCTCCCACAAGTGAGACCATACGAGGTTTGTCTTTCTGTGCCTGGTTTATTTCACTTAACGTAATG
ATCTCCAGTTCCATCCCTGTTATTTTGAATGACAGGACCTGATTCTTTTTTGTGACTGAAAAGTACTCCATTATGTATATATACCACATTTTCT
TTATCAGTTCGCCTGTTGATGGACACTTAGATTGCTTCCAATTCTTGACTATTGTGAATAGTGCCACAGTAACCATGACAGCGCAGATACCTCT
TCAATATACTGATTCCCTTTCTTTAGTGGGATTGCTGGATCATGTGGTAGCTCTATTTTTAGTTTTTTCACCAACCTCCAAACTGTTCTCCATA
GTGGTTTTATTAATTTACATTGCCACCAGCAGTGTACAAGGGTTCCCTTTTCTCCATATCCTCACCAATATTTGTTACGCATGTCTTTTGGATA
AAAGCCATTTTAACTGGAGAAAAATTATAATTTCATTGTAGTTTTGATTTACACTTCTCTGATGATCAGTGATGTTGAGCACCTTTTCATAAAT
TTGTTTGCCATTTGTATGTCTTCTTTTGAGAAATATTTATTCAGATCTTTTGCTCATTTTTAAATCAGATTATTATATTCTTTTCCCGTAGAGT
TGTTTGACCTCCTTACATATTCTGGTTATTAATCCGTTCTCAGATGGTTAGTTAGCAAATATTCTCCCATTCTGTGGGTTGTCTCTTCACTTTG
TTGATCCTTCCTTTGTTGTGTAGAAGCTTTTTAACTTGGTGTGATCCCATTTGTCTATTTTCGCTTTGGTTGCCTGTGCTTCTGTGCTTGTGGC
AACCATTATGCAAGAAATCTTTGTCTAGTCCAATGTCCTGGAGAGTTTCCCAATGTTTTCTTGTAGTAATTTCATAGTCTGTGGTCTAGATTTA
AGCCTTTAATCCATTTTTATTTGATTTTTTTAATATGGTGAGAGATACCGGTTTAGTTTCATTCTTCTGCATATCCATATCTATTTTTCCCAGC
ACCATTTATTGAAGAAACTGTCCTTTCCCCAGTCTATGTTCTTGGCACCTTTATTAAAAATGAGTTCACTGTAGATGTACGGATTTGTTTCTAG
GTTCTCTGTAATGATCTGTTGGTCTATGTGTCTCTTTTTATGCCAGTACAATGCTGTTTTGATTATTATAGCTCTGTAGTATAATTTGAAATCA
GATAATTGTGATTCTTCCAGTTTTGTTCTTTTTGCTCAGGATAGCTTTGGTTATTCTGGGTCTTTTGTGGTTTCATATAAATTTTGGGATTTTT
TTTTTCTATTTCTGTGAAGAATGACATTGGTATTTCAGTAGGGATTGCATTGAATCTGTAGATTGCTTTAGGTAGTATGGACATTTTAACAACA
TTGATTCTTCCAGTCCATGAACTTGGAATATCTTTCCATTTTTGAGTGTCCTCTTCAATTTCCTGCATCAGTGTTTTACAGTTTTCATTATAGA
TGTCTTTCACTTCTGTGGTTAATTCCTAGCTATTTTGTTTTATTTGTAGCTATTGTGTAAGCAGTATTACTTTCTTGATTTATTTTTCAAATTG
TTCACTGTTGGCATACAGAAACCCCACTGATTTTTGTTGTGTTAATTTTGAATCCTGCCACTTTACTAAATTTGTTTATCAGTTCTAATAGTTT
TTTGGTGGAGTCTTTAGGTTTTTTCCAAATAAGATCATATCATCTGTGAACAAGGATGATTTTACTTCTTTCTTTCCAGTTTGGATGCCTTTAT
TTCTATCTCTTGTCTGATTGCTCTAGCTAGGACTTGAATAACAGTGGTGAAAGTGGGCATCCTTGTCATGTTCCAGATCTTAGAGAAAAGACTT
TCAGTTGTTCCCCATTCAGTATGATACTAGCTATGGATATGTACTATATGGCTTTTATTGTGTTGAGGTTTGATCCTTCTGTACCTATTTTTTG
AGGGTTTTATTATTATGAAGGACTGTTGAATTTTGTCAAATACTTTTTGGCATCAGTTGAAATGATCATATGGTTCTTGCCCTTCATTCTGTTG
ATATGATGTGTCACACTGATTAATTTGCACATATTGAACCATCCTTGCATCCCTGAGACAAATCCCACTTGGTCATGATGAATGATCTTTTTAA
CGTGTTGTCGAATTGGGTTTGCTCATATTTTGTTGAAGATTTTTGCATCAAGGTTCATCACCGATATTGGCCTATAGTTTTCTTTTTTTGATGT
ATCTCTCTCTGGTTTGGTATCAGGGTAACACTGGCCTCATAGCATGAATTTGGAAGTGTTCCCCTCTCTATTTTTTGGACTAGCTGAGTAGGAT
TGGTATTAGTTCTTCTTGAAATATTTCCTAGAATTCAGCAGTGAAGCCATCAGGTCCCAGCCTTTTCTTTGCTGCAAGACTTTTTACTGTGGCT
TCAATTTCATTACTTGTTATTGGTCTCTTCAGGTTTTGGATTTTTTCATGGTTCAATCTTGCTAGGTTGTATATGTCCAGGAATTTATCCATTT
CTTCTAGGTTTTTCCATTTATTCTTTTTTTCAAAGTTTCTTAAGCTTTTGCTCTCCATACTCACTGTCTTGTAGCAGCAGAGTTCCAGTGGTAG
CTTTATGGAATTTATTGTTGATTTCTCATCCTTATACATATTCTGAAATTCATGACATTCTCTGTTTTTTACTATTGCTAAAGGCATGGTTTCA
CATATGGCTTTCTTTGTGCTTCTTGCTGGACTTTGGTTTTGGGGGAGTAATGTATGGGAAGATTCATATCCTAGTAGTTACTATTATCCTCCAG
ACCTAGAAAGTCCCCACTACAACATTTTTTGAACTTTAGTGAGACCTCCCTGCCATCCATTGATCTTCACTGTGTTCCTGTTTTCACTTCTTTC
TACATCTTTTTCCGTGACCTATTGCTGTAGTCATTTTCTCGCAGGTTCCGTAAACTCCCCTATTGTTCTCATCATTCTTATTTATCTGGAAAAA
CTCTAATTGAAAATGACCTCTTCCTCTGTGTTCAATCTGCACATGAATATAGCTGGAGAAAATCATGTAGGCTGATTTCACTTTAAATTTATCA
TCACAGGCCTCACATGGGCACTTATATCTGCCCTGGAATTTAACTGTGCTTCTCTAATACACCTATAAGATTCTCTACCACCACAGCCTGGAAA
TTGCTTACATCTTCCTGAATCCAAATCTATAAACCTAATTTGCATCCATAATTTTGTCCTTAGAGCACAGGAGGTACTCCTTTTCTTCACATTG
AATTCTTCCATCTGAGCTCTGGATTCCATTTATTTCCTCTACTTAATGAGTTTTGCTCCTTCAGTTATGCCTTCTTTTTCTTGTTTTGTCAGCT
TTACCTTCTCTCCTACATGGTTCTCACCACAAATAAACATGGTCTAGTATCTCTGTCCTAAAACGCATGCCTCCCCAGACCTCATCTCTGCCCA
CCCATACCCCAGTCCTTTTTATCATTCCCATTCCTCTGCTCTCCTTTGAAGGCAGACTTCTCTAGGGAAAAATAGTGGTAGGAGTGTGTATGAG
TGTGTGTGTGCGTGTGTGTGTGTGTGTGTGTGTGTGTGTGTTTGCAATTCTCCAGTCAGCACTCTCCAGTAGATTTTTCTGTGATGTGGAAAAT
GCTTTGTATCTTCACTGTCCAAAACAGTAGCCACTACCCATGTGTGGATATTGAAAACTTGAAATACACCTAATGTGACCAAAGAGCTGAAGTT
TAATTTTATTTAATTTTAATTAAATGTAAAAAACCACACATGGCTAATGACTACAGCATTAGTGCAGCTTCAGTAAAAGAACCTGGACTCCTTT
TTAGCAGTAGCTTATTTCAGGACTTGGGCACGGAAAATATGACATGAGTCTGGAGCATCTTGGGGTCTCAAAAACTAAGAAAGTGCTAAAAAAT
AATGGGGATATGTAAAAAGGACATTGAAGCCAATGTAAAGGGACTTCAAATTACCAAATCTGGAACAATTTGAGCTTCAGAATAAATAATAATA
GTGGATTAAAACTTATAGAATAAAATAAATATAAGCAAATACTGGTACAAATAAACAATTAAAAACTCAAATACTTGGTAGGGAGAAAGGACTG
ATTTTTCCTATAGTAGAATTCTAGTTAATGAATGTAGGAGGAATGATGCAAATGAAAATCAACATTGGCAAACATCACAATAATAAGTGGGTGA
GGGGACAATTCAAAAAATCATCTATGGATGCTAAGTGAGCATAAGTGTGGTGACAAGATGTCTGATAGATGCCACCTTAATTGAGTGATCAAAG
TTAACATCACCAATAATGAGACATCCAGTACTTCCCAATAGGATGCATTGAGAAGGACACCAAGATACATAACCTCAACCTAATCATGAGTAAA
CATCAGACAAACTCAGATTAAGGAATACACTACAAAATAACTGGCCAATACTGGCAAGATCACGGGGCAAAGAAGGAAAACTAATGAACTGTCT
CAGGATGGAGAGGAATAAGAAGACATGACAACTAAATGCACTATGGGATCCTGTATTGTGTCCTAGATCAAAAAAGGACATTGGTGAAATTTGA
GTAAGGGCTACAGATTTCAGTAATTCTGCTGTGGTTCTACAAAACGTTAACATTTGTTGATGAGTATACAGAAACTATTATTTTAACAGCTTTT
TTGAATATCTGAAATTATTTCAAAATAAGGTTTTTAAAATTGCCTCTACCTTGTGCATTCAACCCAGGTCTGTTTTCTCAGTTTCAGATTAATA
TGTCTACTTTGATGAATCACATTGCAAACTTAACACATTTAAAACTGAAGTACAGGGAGGCTGAGGCAGGAGAATGGCGTAAAACCGGGAGGCG
CAGCTTGCGGGGAGCCGAGATTGCGCCACTGCACTCCAGCCTGGGCGACAGAGTGAGACTCTGTCTCAAAAACTGAAGTACTGATTCTCATGTC
TATCCAACTCCTCCCCCAAAGTCTTAGCAAATCTTTAATTCCGCTCAGTATGTTTTTCATCTCTAGATTTTTCCATTTTTATTGTCTTCTGTGT
CTGGATGGATCATATGGAATATAATTATAACTTTTAAATGTCTTTGTCTGCTACTTCTGTCTTCTTTGTCATTTCTGATTCTGTTTCTATTGAT
TTTTCTTTTCATTATGGATCATAATTTCTTAGTTCCTTGCATTCCTGGTTATTTTCTGTTCCATCCCAGACATTGTGAAGTTGCTGGATATTTT
AATATTTCTATGAATGTACTTGAGCTTTGTTCGGCACTTGGTTAAGTTACTTAGAAACAGTTTGATCCTTTTGTGGTTTACTTTTAACTTTTGT
TAAACAGACAAGCACAGTCTTTACTGGAGCGCTTATCTTTTCCTACTATTAAGGCAGTACCCATGAATTACCAGGTTTTCTACTCTGCCTGATG
GTACCTCTTGCTAGCCATGTGTGGGTTTTACGAACTATTCCCTCTATTCTTTTCAAATGGTTCTTTCCCCAGCCTCAGATGATTTCCTCATATA
TACACGTATCAATACTCAGCTGAATACTCAGTGGGGATTCTTGTAGCTCCCTGGAGGTTTTTCTTTCTCTGTGCAACTTTCTTCCCTTTAGTAC
TCTGCCCTGGGGTCCCTAGTCACTTTGACCTCCTCCTGCTGACAGCTTTGCCTCTTCTTCTCAGAGAAACCATTTAGCTGTAAGCTGGGGCAGT
TGGAGGGCTCACCTGTCCTTCACTGCTGATTTTGTTCAATTCGTTTTGGGTGGAGAGTAAATCTAGTTCCTGTTACTCCATCTTACCTGGATGT
AGAAGTTAAAATTTTCTTTTAGTTTAAAAAAAAATCCAAAGAAAAGAATCAGATCAGTCTTATTCTGAACTGTTTATAATATTTTCTTACCTTT
GTAAATTTTAGATACTATATTGTAAATGCTTTATATGTCACATATAAATAAAATATCAAATTCCTTATTGAGGTTATTTTCAACATTTGTTTTC
TGCCCATCCATGATTTATGATATGAAATTGCTTACTTACAGATTGGAAGAACAGTGCTATGCTAGGTAATTAGAAAAATAATATTTCCAAATAT
ATGTAATGAATAACCTATATTTAATAATATTACTTAAGTTTTGCAGAAAACTCAATTTTTAACTGTTTTATATTTAAAAATTAAGAGTACTGAT
GATTTCTGTAGGGTGCACTTCTGGTTTATGGGCATCCGTGTTTTGGTTTGTGGGTATTCTTATCGTTTCATAAGTTTTTTATAAGTCAGTACTG
CATTTTCCTCTGCTTTTTTTTATACCTCATAAAATAGAAAATTCGATTTCTGTCATTAAGGTTTCTCATAATTGGTGTTTATGTGAGATAAATG
TGGTATTACAACCACACTAATATTGTATTTGGGGGCACAGTCGGAATGTAGAAGGGCACTGGACTTCAGTAACAAAATTAAAAAGTAGAAGTTG
AGTCTTGGCCAGCTTCTTACAAGTTACGCTACCTCTTTGAGCCTTACCTTGTAACTTCCTCACATAACCTGTGAGCCTTACTTCTTTTAAATGG
CATAATTCTCTTCTCAAAGGCAGTTGTGAGAATTACGTGCATTTTGTGAAAGTGCATAGTAAACTGCAAATGCCGTGTCACATATCATTTTGAG
GTTTAGCCTGAGAAGGGTAAGTTTTAAGCAGTATGTACAGTGGGAAAATCTGACTGTACCTCAACCCAACATTAAAACCCAGAATTATCTTGGC
TTGTGTTTTCTTTGGCTAGTTTCAAAATTTCTTTCTTTATTTTTTTATGCTCACCTATAATAGAAATGAGTCGTATTATCATTTCTGTCTTTAC
TCTGCAGTATACCAATAGCACTTCACTCACATCTGTGACCTCTCCGTTGTGGAGAGAAATAAGGATCTACTCAACAGCTTGCATCTTTCAACAA
AGAATACTTTTCAGCAACACTCATGAGTTTCTCTTAGCCATTAGAAAATCCAGACCCTTAGACATTAAACTAAACTCATAAACTCAGTAATATT
TAGTGAGAAAATTGGAGGAACAAATCACTAAAGTTAGATTCTGTGTTCTCTGTAAATGCCAACAACTGTGAGCCATGGAGTAGATTAGATGACC
TTGGTTTTTATGGTCTAAAACATTTTCTACAGAATGGTGCTTACTGAGGAGCTGTTTACTGAGAATTGGAATAAAAGGCTGCAGAATTGTGTGA
AGCTTGCCCTATATTTATGAATCAGATTTTTAAGTGGAAGCTGCAGTTGTTTCCTTGTTTGTCATTCATGCTTTAGCAGACGATATGATGCCAT
AGAGCTGGCTTGGTTTGTAGCTTCTCATTTAGCGTTTGCACCACTTTCTTTTCCTTTTTAGAGGCCACAGCAGTTTTGATGTATTATCAAACCG
CAAAATGTACCTTGTTGGGTATTTACCACAAGGAAATGTGTTGACTATTACACATCCCTTATCTACAGTGCTTGAGACTGTTTTGAATTTCTTA
TTCTTATTTTTTTTTTTGAGTATTTGCATTATATATACTTGCCCATTAAGCATCCCAAATCCAAAAAGCCCAAATTTGAAATGCTGCAGTGAGC
ATTTCCTTGGAGCATCATGTCACTGCTCAGAAAGTTTTGAATTTTGGAGCACTTTGGATTTTTGAATTTGGAATGCTCAGTCTGTGTTTGATAG
TTTCATTAGTGATTGTGGCATTCCTGTTTAGTCTCATTTTGAAAATACAGCATAAATTTAAAAATGCATACATACATATGTCCATACATATTAC
TACCTATTCCATATAGGTAGTATATAGCCATACAACTGCAGACACATGGAAAACAATTCTTAACCTTTTGAATGTATTAGTTTGTGTTTAAAGA
TAATGTTTTATCTTGTTTAACTGGAATCTAATATGTTAAATCTCTTTTATTCTACCTTAGCATCAAGGCATGCCATGGGTTGCCTCTCATAAAT
GGCCAAAGCTGTGACCCTTATGCAACAGTTTCTCTAGTGGGCCCTTCTAGGTAATATTTATTGAATTATTATTACGTTTTAAAGTTTTGATGTT
TGATTTAAAAATTTAAAAAGTAAGATTCTACGAGCAAATGATAAATAGCTATTTATAACACACAGTAATAAATGCTTTGAAAGAAAAATCTTTA
GTGAGTTCTATTATACACTTGTCAATAACAAAGCCCTCTTTTTACAGACAGAAATTGGTACCCCATTTTAAATCTGCCATGCTCTTAGGCTTTA
AGGAACAACTTCAGTTTTATACTAGTACAATGTAGTACTTTAAAACATAAACTTGACTTTTCATGCATCAGCATTCCTACTTTTCAAATATCCT
GAATTTTATGCATGTAAAATCCTGTTTTAATGAATGCCAGTTCAGAATAACAAGACAATTTATATATTTTTATCTGGAAGGCAGCTCAGGACTG
AAAATTGCCTTTTCTACTCATGAAATCTCTCCTCTACAGAGAGAATTCCAACTGTAGAAAAACTGCTGAAGTACTAGAGCATGGCAAAGTGTAC
CATATCATTGATTTAAATCATTTATAATAATTTGTGAAAAAACCTATAGCACTTTGTAATACGGTGTGTTATATGACTTCATTATGATTTCTTT
AAGGAACTGACACATCTGAAATATGTCTACAACTTGAAGGTTTAGAATCAGGAAGCAAGAAACACAGTATTCCCTCTACACACACATACATGCA
TACATACACACCCCTTTTCCCCATCCCCAGAATCTCTAGTTATTCTTACTTTGTAGAAAATCACTACTAAGTCACTTTGAAAAATGTATTTATG
TATGTATGTAAAGTACCTAGTACCATGCCCGTATTTATTAGGTGCTCAAACAATAGTATCCTATTAGCAGATACTCAGTCGTATGACATTAAAT
GCTATTAGAGAGATTTATTTTTGTGTGTTAAAAATGTTCAATCTAGTGGAGCTATTTTTGTTTTTTTTAATAATCTCTACCTGACAAATTATTT
GGTAACATACAGTTAGAGACAATTTGGCATATGAATTTGTGTTTTAAAAAGTCTGGCCAGGCGCAGGTGGCTCACGCCTGTAATCCCAGCACTT
TGGGAGGCCGAGGCGGGTGCATTACCTGACGTCAGGAGTTCGAGATCAGCCTGACCAACAAGGAGAAACCCCGTCTCTACTAAAAATACAGAAT
TAGCTGGGTGTGGTGGCTCATGCCTGTAATCCCAGCTACTCGGGAGGCTGAGGCAGGAGAATCGCTTGAACCCTGAGGCGAAGGTTATGGTGAA
CCGAGATTGTGCCATTGCACTCCATCCAGCCTGGGCAACGACCAAAACTCCATCTCAAAAAACTAAAAAATAAAAAGTCACTGTTGGTTTTGCT
ATTGTACTGTAGTTGTGTAAGATGTTAACACTGGGAGAGGCTGGGTAAAAACACAACATTTCCTTATGCGTTTTTTGTGACCTCCTCTGAATCT
ATAATTAAATTAAAAGATTTTTTTAAGAAGTCTCTATTATACCTGAAGCCCAAGAAGTAAGCTGAGTTAAGAATGTTTTTCAAGTTCCTTGGGT
ATTTTTCAACATACATGAAGGTGCTCTAAATCTTATTGTCTTCCTTCTATTTATTCTGAGTTGCTTATAGAATAGAATGTGTTTTTAAAACCCC
TGAAAAAGGTTGGCAATCTAGTGTGTCCGTAGAGAGAATAATACAATCTTGGCACATAGGGGATGCCTAAAGCTTATATATTCTTTGATTTGAG
TTTATAAGGACAGTTACTTTTTTCCAAGCAATATTTAAAACTGAATCTTCCCTGAGTCACCTTAACTTTTAAATGGAGGTCAGGCTCCCTGGTT
GGAAATAGCATGTTTTATAATTGTGTACTGTTAAGTTCTACAAGGTAAAACTCTACCACATTGGAACAGGAATCACCAAAAGAAGACAAAAGTA
AAGAAGAAAACAAGCAATCCGCAGTTTAATGAAATCTTTTATTTTGAGGTAATTTTTTGTTTTACGTAAATGTTAACATTAAATATGTAATATT
TAATGCTAGTTGATTTCTTTTTTCAAACCACAGTCATAGTAATCATTTTATCAATTAATGCAGATAACCATTTCTCTCCTGAAAGCAGTAGAAC
CATGGTGTGATGCAATGGATGCCCAATATATAAAATGAATAAAGCCACAGCTCTAATTTGGGAGCAGGAACCGCCAGGGCCATACCCACTCCCC
AGTCATCCTCACTATTCATGCCCTTCCTTTGCACCCTCCCTTACCCTACCCAGCTCCCTACCTAACACATAGTGGCCACACGATTCTTCTGCAG
GGTACCCTTTGACAGTCACTAGTGAAGTAAGTCTAATTATCACTCAAGAAATCTGACTTCCAGTCAAGTTGACTGAAAGACTCACTAAATGTGA
CTCAAGTTTCTTATCCTGTGAAAGAATAATCGCAACGGAAACTAGCAGCACTACCTCACTCAGAGTTTTTGTAAGACCCAAAGCATGTGAGAAT
CCTTTGAGACCATAAAATACTTTATGATTACAGCTTTATTATTACTTTTTTTTTTAAGCCTAATTCATTCTGTGGACGCCTGCTTATCTTCATA
GACAGCTTCAGCTCCCTTGAAGTTGTCCCAAATACAGTTTGGACAAAAGCTTTGGAGTGCTTTTCTTTGGAGCATTTGTTAAAGAAATCCTTTT
TCATATTATTTGCAAACTACAGAAAAAAAAAATCAAACTCTCAAAAAGATTGTATTCTAATAAAGCATGTTGCAGTTCATATCAACGTGTAGGG
AAGGTTTGAAAAAAGGAGTTACATTTTGTGCCTTTTCTTCTGTGTGTATTTTTCTTGGTATTTCACAGTGGAGAGAATAACGGTACCTCTAGTC
ATGCCTGATTGCTGTACTTTAATAAAGGAACACAGGCCGAAGAAAATGCCAAAAGTGAAGCAGCAAGAATTGTGGAGGGGGAAGCAAGGGAGAA
AAAAAAAACAAAAGAAAAAGTACCAATGTGAGACTTTTCCATTTCCTGGCTAAGGACTTGGTGCAGTTGTTCTACGTTTATTAATGGCGGTTGC
ATCTAACCTGGAGAAAACAAAGAAGGCAGCAATGTAAAGGGCCGCTGACTAAAGGCACTCACACTCCCCATTAGCCCACATACCTTAAACCTCA
GCTGGATGGAATCTACTCTGCTTCATGGATCCTTACAGTCAGCAGATGACCTTTTAAAAGTAGAAGGCCTGACCTGCCTGCTGTAGCAGGAGTA
CTTGTTAGGCCTTGCAACATTCATTCAGAGGGCCTTTAGTTGGTTATAAAGATAGATACATTTCCATCTCTCTCCTTTAACTGAAGCAAGGAAC
ACAAAGCAGACATATGTATGCATATACACAACAGTAAAATGCAGTAGATATTGGGGAGGTCAGATGAACTCTGCTCCATTAAAGGCTTGAAGAA
ATATTGGATTTTCTAAGATTGAGGAAAGAGAGTATTAGAGATGACGAACATTTTGAGCAGAGGCACAGAGACAGAAAAGCAGAAAGTTATGTCC
AAAGTACAGTGAGTACTGATTTTACTGGGCTTGTGTAAGGAAATAATGGAAACTAAGATTACAAAGGTAGAGTAAGACCAGCTGATGAAAGGCC
TTGAGTGCCTAGTTATGGTGCTTAAGTAAATGATTTTGAGCCTATAAGTTATGAGTAAAACAGTGTTTGGGAAGGAATTAACTATGATCTGAGG
TCCAGGTGGCTTAGGGAGAAAGAACAAAGCTGGGGAGACTGTTGTACTTTAGGTGTAAGAAAAATGGGGAGGGGATGGGGTATCTTTCAAGAGA
GTGCTATGGAAATAGAGAGGAAGAGGCAGATAAAAGAGAGAATGCAAACGGCAAGGTAACAAATTGGTTATAGAAAAAAAGAGAAAGGAAGTAG
TTGAATGCTGCCCCCAAGGTTCCAAATCTGGAAAAATAGAAGAGTAGGCAAGAAGGAAAATAAAAGTGGCTTAGTTTCGAGCATAATTAGCTTG
AGGGCTGACATTTTAAATCCTCCAGTAGGGAGTTTATGACTGAAACTTAATATGAGTGATAGTTATTGACCAACACTCAAGGTTTGTTTATATA
AACACCCAAGAAACCCAAGAGAATAGAGGTATACAATACAGGAGAAAACTGAAGAAACTAATTTCTGCGACTCAACTATTTAACTTTCAGCATG
TCATTTAATTTCTGTACTTCAGCTTCCTCATTTTTAAGTGAGGGTAATAAATCTAGCCCTCTCCGCCTCTGAGCAGCGTTCTGATGAACAAAGG
ACATAATGCATGTGAAAGCACTTTGTAGATTGTACAGCACTGTGCAGCTCCAAGTTGTTTTTAGTGCAAAATAAAACATAAAATTTTGAAGAAT
AATTTATCTATAGATTCTGAAAAAGAAACAAAGACAGCAGCATAAGGTTGCCAAGGTAGTGCCCAGGTAAATTCTATAGAGTGTTTCTGGGAAA
CGAGATACAGTTGTATGATCACTAAATTAAATAGGAGATCAACGGAGGTAAGAACTGAGAATTTATCCTCAGATGTGGTGATGAAGAGAGATTT
TAGGAGAGTGATCTGTGAGGAAGCCAAGATTATAAGGAGTTACTATAGTGAGTGGGTGGTGAGGAAGTACAACCTGTAAGGATGGATTGCAGTT
TTCAGATTTGTCTAAGAAAGGAAAGAGATAGGAAGACCATAATTTAAGGGGAGAATTTTTCTTAGACTTGAGTGAGATGAACAAGCTGGTATTA
TTTTGATTTTTTTTTTACACTACCTTATTCTTTTCTGCTACTTCCATAGTTGCTTTTATATTCAGCTATTGAGTAAAATATTACTTGGACATTT
TCCTCTGCTTTTATCCACAATTGAATATTTTGCAGATGATCATTTTTAAGCTGTAGGAAAAATGTATTTTTTTAAAAAAAATTTTTACTAAAAT
ATTTTGTTTACACGTAACCAGATCCAGTAGTTACACCAGAAAGTCCCAGTTCCAGGTAGAAGAGGAGGACATTGAAAAGCTAGAAATCAGGTAT
GTGCCTTGGGGTTTTACAGAATTGCTTTTTTGTATACCAAAAGAAAATCCTAGATTCAACCAAAAAAAGTAAACCAACTTTAATAGAATTCTCT
ATAGCAAGTTGGTTGATGAAACTTAGAGGAATATGCAACTAATTTAAATCCAGATTATCTTTCCAAAGTCTGATTTGTTTGCACAAATAGTCAA
AGTGTATTAATTTTATAAACAAAGATGATCTAATTTTCATCTGTTTTATTGTATTGATTTTTAATTTTTCCCAAATTCTTCCTTACATTTCTGG
ATTATAGTGTAATGGATGGAAAGGAAGTTATATCTCAGCCAATTTAGAAATTTTTTTTTATTCTTCTTAACATATCTGCTGTCTAACAGAAATT
TCAGTGAGGTAAGAAGATAATTCAACAGGTGCATGTGTTTGAAGTTTGTGCTGTTTCCTGGGTCTTAAATATCTATGCCCTTTGCGAAGGAAAA
ATCTTATAAATGCATGTGTAGGTGTGATGTTAACAGTGTGTTCATCTAGATGGTATATTAATCATGTGTTTGGAGTGATCAGGTAATGTATTTT
TCTAATTTTTCTATATATATATTAATTGGTGGTAATCTAGAGAGAAACTGTAGTCTTATTGTAGTTTCTTTCTGTAGTCCTTATTGCTATAGGT
TAAAATAAAGCCTCGACTTTCAAACTAAGACAGTTTAAGTGCCTTCTTTCAGACTATTTAAAATTACTTTCTGTGCTTTTTAAATAAAAGCTGC
TGTAGTATTTTGCTTCTCTAGTCTGGATAGTGAGGTATCTATATGGTGTTGCTAACATCTTTTAAGCATGTAAAATTAATGCTGTAATACAGAT
CAAAGTAATTTGAAGCTGTTTTGTGGTGTGAACTCTCTTAAACATTGTTTGCAAAACATAAAGCCAGTTTTCAATTTTCAGGATCGACTTGTGG
AACAATGGAAACCTAGTCCAAGATGTTTTCCTAGGTGAGATTAAGGTTCCTGTGAACGTATTAAGAACTGATTCCTCTCATCAAGCCTGGTAAG
GGCCCACCATTTTAGTGAACTCCATAGTTTAATTCTCTTTAGTACAGTATATATATCCAAATAAATGCTTACTCTGAACATATTTTAAATTTGC
CATGTAATAAGCTAAACGTCATTAATATTGATATGTTCATTAATTCATTGTTGCAGAAGTAAATTGTAAGTTTTATGACTTGACCTGTGGTACT
GAGATAAAAGAACTGAATCATCTAGGAATATTTTTCTGTTCACAAGTGAAGACGACTCTTAAGGAGAAAAATGGGAAACATTTAAAACATTACA
TAAGAGTTGTTCATGTAAGAAAACGCTTGGTCACAAATCAGTGTCTGCATTTCAGAAGTTTTATTAGTGGTCATAGGAACTGTGGGTATTTGAA
AGCAAAGGATTTTATTTAGGGGACTCAGTTGTTTATGGGAACTAACCACTGTTCTTCCTTCACCACACCCCATTGAAATGTACTGTAGATGGCC
CTAATGTTACTTTTTGAATGTCTAAGGTCTTATGTTGTTGACCTCGCACTTTTTAGGGCTCTTCCTTTTCAATAATGACTATAATATTATTTTA
GTTTTCAGTGACTCTAATAATAACATTACACTGATGTTAAATTCAAATTAATGTTCAGACATTACATTGACCTAGTTTCCTTAGCAGAATCAAT
GTGAAACTATTGTTTTTTTTCTGTCTATAGCACTTAAAAGCGTGTGTATCCTTTCAGTATGTTGCCCAGCATCTCTCTTGCTAGATTTTGGTTT
CAACTGGGTATAAATGAACATGCCTTTCTCATTTGCCTTTTAAGGAGCACTTTTTGGATCTCAGTTTCTGCTGCCTCAAAGAGCAGTGTTTAGG
TGTTTCTTATGGATATCAGTTTCATCTGTTCCTAAAAGAGCCTGCCTCTTCTGATTGGCCATCACACATCTTCATTTTAGACTCTTGTCCTAGA
GCAGTTATCACTTCTAGACTGATATGGAGACTCTCAAAATTTTTACTTTATTTTTGCCTCTCTATAGTCCAAGGCAGATCTAAGATTTATTGGT
CTTTATGTTGGCATTTTGTGTATTATAGTTTGTAAAATGCATCCACAGAGGGGGAAAAAAATCAAATCTAGTCACTATGAAAAATTGATGAAGA
TTTGGGACCTGTCATCCTCCCACTACACCATGTCTTCCACGTTCCATCAAATAGCATACATTTTAAAATGAAAATTTCTGAGCTCAGTTTGTCA
AGTTTTTAAAATTATGTGCCAGACCACTCAGTACAGAAGAGAAAAACACGTTGTAGTGCCTCCGGGTCCCTCAAAATGAACTTGAAATGTGCCA
CATAATCTGTTTTTTAAGTAAGACTGTCTCCAGAGATGCCTTCCAAATTTGGCAAAACTTTGTGCAGCTTCCTTGGCAGTATGTAAGAACATTA
ACAAACTAATTTTATTTTTATAGATGTTTTTGGCCCATGATATTTTAAAGTAACCTGCCAGAAATATAAGTGCTGAAATTTATATAGCATGAAA
TAGGAACATCTGGTAATTCATATATAATTCCACAGTTGTAAGGAAACATAAAAATGTGAATGCCATTTCTTTTAAGTTGGAAATAAAACTCTTA
ACCTCCTCCATCAATTGTAGAGCCATACCATCCCCTTGTGGGGTTGTTAGTAACTGCATGAAAACTCAAGAAAAGATTTATTTACCCTCAGGAT
TTCAGGCAAATTCTTTCTAATGTTGATGTTAAGAGAGTAATCATCAGTAATTTAATTCTGTTGTTTGAAAAGTCTCAAAATAGATTAATTTTTA
AATTTGAAAGTAAGATTCAAATAATAGATAAAATGAATTACTGGGTTTTTCTTTTCAACAGAAGCACTTTATACATGATTTTAGACTAGATGAA
TATTTGATATCATTTAGCTTTCCACTTTGTTAATTTTGACACCTTTTGTCCTCAAACTTTTTTTTGTGGGGGGGCGGTCTTACTTTTTTTGAGA
CAAAGCCTCACTCTGTTGTCCAAGCTGGAGTCCAGTCGCATGATCTCGCCTCACTGCAACCTCCACCTCCCCGGTTCAAGTCATTCTCCTGCCT
CAGCCTCCCAAGTAGCTGGGACTTCAGGCACACACCACCATGCCGAGCTAATTTTTGTATTTTTAGTAGAGACGGGGTTTTAACTATGTTGGCC
AGGCTGCTCTTGAACTCCTGACCTTCGTGATCCGCCCACTTCGGCCTCCCAAAGTGCTGGGATTGTCAGCCATTGCGCCCACCCTTTGGTCTCA
CATTTTTAACGTATCTGTTTAATATTTGAATTTAGAAAATATAACAAGTCTCCTGTNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNTCCATTCTCCTGCCTCAGCCTCCTGGTAGCTGGGATGA
CAGGCATCCGCCACCACACCCAGCTAATTTTTGTATTTTTAGTAGAGATGGGGTTTCACCATCTTCACCAGGCTGGTCTTGATCTCCTGACCTC
TTGATCCCCCCGCCTCGGCCTCCCAAAGTGCTGGGATTACAGGCGTGAGCCACCGCACCTGGCCCACGTTCAGTTATTAATAGAACTGCTTCTT
TATTCTCTCATTTACATAGCATTATGTACAAATAATTGCACTTTGGGTTTGTCTACCTCAATTTTAATTACACAGACCGTATCGTTAGAATTGT
TTAAATGCTGATTTTTTTCTGTTAAGGGACTGTCAAAACTGCTTACTCTTGAGAGCATAAGGATTGCTTTTATGCCTGTGAATGAAAAGTACAT
GTTTAGAGCAACTGAATGCTACTTTCAGTTAAAAATTTCTCTTTATTCCCTGTTTCTTTTTCTTTCAAACGGAAATATTTCATTCAAAAACTGA
CATATATATATATGAGTTTTTTGTTTTGTTTTCACTTAGAAATGCCAGATAGTCCTGAACTATTTAACAAATCTTGAGCCTTTTCTCTTTAGCT
CTAATTCATACAGAATTATTTAATTCACAAGCATCCTTCAGGCAGTTTATTAAATTGAGCTTTTGTAATAATATTTTAAAATAATCACATTTAT
ATAGTTATACAAATGACTATTGATAGTTATTCATATATTTAAGTGGTAATAGTTATACATCACATATATAAATGTTATATGTGTAACATTTACA
TAGTGATTACTGTGTGCCAGGAACTATTCTAAGTACTTTACTTATAATAACTTTTTTAGGGCTGTTCTAGGGTTTTAATGTGTTAAGACACTAT
TAATACTCCTTATTTGACAAATGAGGAAAAAGCACAGAGAAATTTAGCAACTTGTCTAAGGTCACACAGCTGGGATTTGAACCAAACTGTTTGA
CCCCAAAGTTTGTGCTTTTAACCAGTGTACTGTAAGTTTGAACTCTGGCATATTTCTGAATATATTTAAAACTCAAAAACATTCTTTTAAGCCA
TTAGCAAAGATTTGAGTGTCCCTTGCTTATAAAATATTATATAAAAGAGAATCAGAGAGTTCCCTACTCATTTAAAAAATCATCTTTTCAAGGG
ATGGGTAAAACAACAACATGTTGTGAAGTCTTTTTTTTTTTTTTTCAAGTAGACATAGATGAGATATTTTGTCTATGGGGATTTCTTATCCTGC
AAAGAAAATCCAAGGAAACAAGTTTAGAAAGTATGCCAGGAGTGAACCCTTCTGGACTTCTTAAGCATCCAAAAACAATGTATGCATAAAAATG
AGTAATTGGTATAGCACTACCCTAATTCCTCTACATAGAACAATGCTATGTTAAGACTGTAGGCCTGGTAATTTTCAGTTATATGAGAGTAAAG
AGGGATGTCATACCCCTCCCTCAGTTATTTGGAGCTAAAGGAAATAAAGTTGCGCTCTGAGCAAAATTTCTGTAGTAAAAACCAAAACAGAGTT
TGTTTTGACTGTAGAGTTCAGAGACACTGTTTAAGATGGATAGGGATGGTCCCAGAGAGGCAGAACCAGTTGTAACTGAGTAAGCAGTAATACA
AGTAATGTTAATCTACTGTGCATGGAAAACATTAGTTCAATAGGAGGAAAAGATTCATCTAGAGTAATAAAAAAGGATTCCAAAGAGGTAGGCA
TGGTACTGCCTATTCAAGCACAATTGAGGGGGTAGGGGATTCTGGTCCAGAAGAATTACTGTTTCCTTGGCAAGACTGTTGTCTTAGCTGAGAG
ATTCTTAAGGCTGCATAAAACTCCTTGTCATGCCTCTGTACTCACCACAACGCCTTTTGATAATTTGCCTTTCCAAATAGCCTACCAACAGGTA
AAAAAATACAGTGAAAGATATAGATGAAAACCCTATGGTAAATGGTGATAATTGTTGTAACCAAGTGACAAGCACATGGGGGTGGTGGTAAGAA
AGGGTGTTCATTATTACTATTATCACTCTTCTCCCTACTTTTGTAAGTGAGCATTTCTATAATAAAATGCATTGGTCACTTAACAATCCAGGTA
CTTATGCCACTTGACTATATTTCTTTTTCATAAATTTAATAATTGACCTGCTTTTAAGTATATCAGGCTCTGCACTCAGTCATTGCAATTCAGG
TGCACCTTATTCTTTTTTTTTATTTTTTTGAGATGCAGTCTCTCTCTGTCGCCCAGGCTGCACTGCACTGGTGCGATCTTGGCTCAGTGCAACC
TCCACCTCCTGGGTTCAAGCGATTCTCCTGCCTCAGCCTCCCAAGTAGCTCGGACTACAGGCCTGTGCCACCATGCCTGGCTAATTTTTGTATT
TTGGTAGAGATGGGGTTTCACCATGTTGGCCAGCCTGGTCTCGAACTCCTGACCTCAGGTGATCCACCCGCCTTGGCCTCCCAAAGTGCTGGGA
TTACAGGCGGGAGCCACCACACTTGACCTTCAGGTTCACTTTAGATGTGCTGATAAAACCAGATGTCTGAATTTGCAAGGTTGCCGAACATGCC
CTGGATTGTGAAAAATTGACCAAAGTATCAATTGTCAGGATGCTTGGTCATCAACCAGCTAAACTTCCTGATGTGGTTTGGCTGTGTCCCTACC
CAAATCTCACCTTGAATTGTAATCATCCCCACATGTCAAGGGCAGGGCCAGGTGAAGATAATTGAATCATCGGGTGGTTTCAAATCCCCATACT
GTGCTTGTGGTAGTGAATAAGTCTCATGACATCTGATGGTTTTATAAATGGGACTTCTCCTGCACAAGCTCTCTTGCCTGCCGCATGTAAGACG
TCCCTTTACTCTTCTTTGTCTTCTGCCATGATTGCGAGGTCTCCCCAGCCGTGTGGAACTATGAGTCAATTAAACCTCTTTCCTTTATAAATTA
CCCAGCTTTGGGTATGTCTGTATTAGCAGCATAAGGACAGACTAAATACACTTCCCTTAGAGGTACTTCTGAGTCACCTTTGCTAACAGGCTTG
TTTCTAATTATTTCATTGACCATATCTTTACATCACTATTTCTTAAATCAGACAGTAGATGTTCTGAAGGTTGATCTTCCTCTTTTGTGGGATA
GTGATGTAATTTCTTCTTGTATTAACCATCCCTTCATCCTTTAAATTTGCCTTGGTTAATTCGCTAGTTGTCTTTTCTGTCTTTATCTTATCTT
TCTCAAAGCTAATAAATGTTCTATTAAAGCCTTCCTCCTTCTATTTTTAAATGAATTAATATATTAGAGGAGGTCATAGCTGATAAATATATAA
ATTAAGTATTAAAACTACTATTTCTTTTATAAAAAGCATCTCAGCATTCCAGCTAAGTGAAGGACcAGTGATAGAGTTATAAAATTATCATTTT
GATGATTAATGGCTCTAAGCCGTGATCATCAGTGGCTGCTGATATTGCAAAAGGACAAACAACCAGACATTTATGCCTCTTGGTAGAACTACAC
AGCATTATCAATGAAGCATTCTTGCCAGAACATCAAATCTGAATCTAACCAAGCCTCTAGATCTAACTACCAATATAGAGGAAATACAGAGAGG
ATGAAGGAATATGGTAAATGACATTGTGGAGGTAGAGATAAGAGTTAACACAATTCCAAAGGTGGAAAACTCTATAGGACCAACAGTGAGGAAT
CTATAAAAGAGACTTAAGAATGCTATTACCAATTCTAATCTATGGACCTTATTTGGATTCGAACAAACTGTAGAAAAACATTTATGATAAGTTT
GGGGAAATTTGAATACTAAGTGAATAGTGATGACTTTTTAAAATTATATATGAAATTATACCTGGAAATTGCTTTAAAATAATAATGCAGGAAC
AGGATGGGAGAGTGAGTGAAGGTATGGCTGAAACAACATCAGTCTTTTATTAAAAAAAAATCAGTGTATGTATTTTTAAATTTTGTTTGGCATT
GCCTTTAGATAGAGAATAGAGAAGATGGCAGCTTTTTTCTCATGTTAGTTAGAAACTTCTAATTGCTTTCAAAAAGTGACCCAAGGCTTCAAAA
CCAAAGCATTCATTCACTCACTCTCTAGTCAATATCCATGGGAATGTCTACTACTAAGCCCATGTGCCCTATTCTGACCTATCTACTAAAAATA
AAATCGTGGGTTCAAACAGCAATGCTTAAAAAAACTCAAACTGCTTAAAAATATTGCACCTGTGAATCTGTTTTAGTATTTTTAGGGAATTACT
TTACTACTGGGCTTTGTAATTTTAAACTTTAGAAAGGCCTTAGCACAGGATTTATAAAACTTGAAAACTATGAGTATAAATAAATAATTCCAGG
CCAGGCATGGTGGCTTACACCTGTAATCCCAGCACTTTGGGAGGCTGAGGCGGGTAGATCACCTGAGGTCAGGAGTTCAAGACCAGCCTGGCCA
ACATAGTGAAACCCTGTCTCTACTAAAAATACAAAAATTAGCCCGGTGTGGTGGCCCGCGCCTGTAGTCCCAGCTACTCGGGAGGCTGAGGCGG
GAGAATCGCTTGAACCCGGGAGGCAGAGGTTCAGTGACCCGAGAGCATGCCATTGCACTCCACCCTGGGTGACAGAGTGAGACTCTGTCTCAAA
TAATAATAATAATAATAATTCCAAACATTCTACATTCTTAGATAAAATATGTAATTTATTTCTCAAACAGTATAGCGGAAATATTGACAACATT
TAAAATGCATTTTAATGTATTTTAAAACTTTTTAAATGTATTTTTAAAACATTAAAATGCATTCTTAATGTATTTTACATGTATTCACAAAATA
TAAGAAAATGCAATTCGTGTCATAATTGTGACATGATACCAAAGGAGAAAATCCCTCCCTTTACTGTAGCAACTGTATACCATAGAGGCAGTGA
GTAAGAGAGGAACAGAATAGAGCAGTAACAAGATGAAAATGGAAAGACAAACTCTTCCTCAGTATAAGTAACCAGTGCTATAAATTTGCCGTGC
CTAGTACAAATGTTGAACAATTGTATAAACAGAGGTTGAGGATTTTCTGCAAATAATAAAAGAAATATCAGTGCAGATTATTCTGGGAGAAGAA
TGGCAAAGAAAATCCACAAAAGCATTTTAGCAGGACTTGGGTAACAGAGGACTGAGAGAGAGAATTTCCCAGACCCAAAAAGCAAATAAATAAT
TTTCCCCAGTCTCTTAGTAAGGAGGGTTGAAATTAAATTCAAAATTTTAATGTTTGCTGTTTATTGCTTTCTGAACACAATTATTTTTAAGTTA
CTTATGGCCTATTAAGCTGTATAGCATTTTCACTTCACAAGACAGTACAATTATTTTTCATTATATTCATAAATACCTCTCTTAAAGAACTCGA
GTCCATTCATTGCATGAATATTATCTACCCGTTAAAAAGAACAAACTCCATCTATCAAAACTAGTTTCAAAGAGTTAACTACCAAGTGCTTTCA
GGTAAAAAAAACAAGATACAGGAGAGTAGAATATGATAACATTTTTGTAAACTGACTAATAGGCTCAGAACTCATTTATAAATGTGTATATATG
CACACATTTTAAAAAATCTATATAAAATTATATAAGCATGAAGAAAATACACAACGGTCCATAGTAGGTAGTTAACAGGTTCCTAAGAGAAGCG
GAGGTGCAGAATGGTAGTGTTTTGAAGATAGGAAAAAAGATAAGAGAAAAACAAAGTTAAAAGAGAAAATAAAGAAAACGTTCCTTAAAAAAAC
AAAAGCATTGATTTGATGACATTGTTTATATATGTATGCATCGTTATCTATTTGTCTATGTGTAGTAACGATTTTTTAATATTAAATAAAGAAG
ATTCTGGTTCAAAGAGTGATGAAAATAGTAAGTGGCACTCCACACAGATTCTTTCTTGGTTCTTGAAGTTTGAGCTTCACTACAAAAAGAATCT
CCATTCAGTTGCTTTTATCTACCCTACAGCGTTTGTTAATAGCAAATCCAAATTTGTAAATTTAATTTGGAGTTATTTCTGAAAATAAATTTTG
TGGGCAATATGATGATAAGCCACAGGAAAAAAAATATGCAGATAAATATTTATAAATAAATTCCTTTAATATATTCTAATTGTGATAGTTTCTG
CAGCATAATTTAAAATTATGCTATTTTCAGTGTCCAAATAAGGATCTTATCAGTGTTAGTATTGATCATCTGGCTGCACATAGTAACTTAAAAT
AATTTTGTTTTAGAATATACTAATAAAAGACATATATAATACACTTTTTCTGCAGTTTGGTTTCTGGTTATAGTTAAGTAAAGCAAAGATTCAC
TATTTTATATACAGCACAAGAAAACGCCTTTTTAAAATAATGAATTATTGAAGAATAAGAATACAGTTAAGTTTTTTTCCTGGATGTTTTTAGA
TATTTTGCTCTAATGGAATAAAGGGGATGAAGTATTTGTAATGGGCATATTATTTTAGATAAATAAATTTTTAAGAGTTTGAACAATTTTTTAA
CTCCTGGAACTCTTGTGATATCTTGAATGACTACCAAACTTTTTTTTTTCCAGTCCTAGAAAGATATATTTTCGCTAAAGCCCAGCAATTCTGA
AGTTTTAATGACCAAATTAGTTGACTTCAATTGTTTTTGCCAACTTAGGCTACTTTAAGAGTAAAAATTGCCTTTCTCTAATCCTGATATAGTC
TAGAAAAAAATAGGTATTTACACTAACAACAAATCATAAATTTCAAATTTAGGCAAGCATATTGCTGAGTATCCCCCCAAAAAGGAAATATGAA
TACTTGATTGTCAATGGTACTTTACAATTTTTATCAGCCTTCATCCAATTTTGTTTTGTTTTGTTTTTTAATTTTAGATTCAGGGGTATAGGTA
CAGGTTTGTTACATGGGTATATTGTGTAATGCTGAGGTTTAGGCTTATTATCCCTTCACCCAAGTAGCGAACAAACTACCGGATGGTTTTTTCA
ATCCCTACCCCCCTATCTCCCTCCCCACTTTTGGAACCCCCAGTGTCTATTGTTACCATCTTTGTGTTTGTCCATACCCAGTGTTCAGCTCCCA
CTTACAAGTGAGAACATAGGGTATTTGGTTTTCTGTTTCTGCATTAATTCGCATAGGGTAGTGGCCTCCAGCAGCATCCATGTTAGTGCAAAGG
ACATAATTTTTTTCTTTCTGTGACTGCGAACAAGATTAGTCTTCAGTTGATAATTGTTTAAGCTAGATAATGGGTACATGAGCGTTCAATATTC
TCTTTTGTTTGTGTTTTTAAATTCCCATGATAAAAAGCTTTTTTTTTTTTTTTTTAAACTCTTAGATTTCTCCTTTTGGTCCTGCAAATTTCTC
TTAAGTTCAAATTTTTGGAAAAAGAATTTACTTTAAAACAAGGTTACTTACGCATTCTCATATGTCTTATCTACTTTTTTTTTTTTTTGAGACG
AAGTCTTGCTCTGTCATCCAGGCAAGCCCAGACTGGAGTACAGTGGCGGGATCTCAGCTCACTGCAACTCCGCCTCCTGAGTTCAAGTGATTCT
CCTGCCTCAGCCTCCCAAGCAGCTGGATTACAGGCACCTGCCACCATGCCGGACTCATTTTTGTATTTTTAGTAAAGACAGGGTTTCACCATGG
TGGCCAGGCTGGTCTCGAACTCCTGACCTCAAGTGATCTGCCTGCGTCAGCCTCCCAAAGTGCTGAGATTACAGTCATGAGCCACTGCACCCGG
ACTGTCTTAATTTTCAGCTAAATGCCTCTTTCTTAAATTTAGTTGGTAAAAATTTATTTTTGATAATACATTGGATTTTAAATGTTTACCTTTT
ATTATCATTTTTTCTAAACCTGCAAGCTAATCAAACTCTTGTTCAACTCCTTTGACATCTTAATTTCTGTAATATCTATGCTTTTTGTTTCCTG
GATAAAAATGTGTTTATAGCTTTGTAATCCAATGTATAACCAATATTAGTTTTCTGAAGATGAGTATCCTGCTACTTATGAAGCTTCCTAACTG
TAAATACTAAGTTGACTTTTTTGG&TTACTTTTTTTATAACTTAACTCTTAACTTGCCTCATTGGCTTGAATGCATTTCCATCACATGGAATCA
CTTATATTTTTACTTTAGGTACTTGCTACAGCCAAGAGACAATGGAAACAAGTCATCCAAAACTGATGACCTGGGGTCTCTTCGATTAAATATA
TGTTATACAGAAGACTACGTGCTTCCTTCAGAGTACTATGGTCCTTTGAAAACTTTGCTGCTAAAATCACCAGATGTTCAAGTATGTTAAGAAT
CTTAAGGATATGATTTAACACGAAACGGCTAAAATAATTCTATAAATGATAAGGAAAAGATTTCAAGAGTATCAAGTCTTATTAAAACAGTAAA
AATTATATACATACATATATATACACACACACATTTCTATTTATCTCAACCAGAGTACTATGATTCCTACATGTGCTCCCACAATCATCTCTGA
TAAAAAATTTCGAAAATTAAATGTCCTGTATAACAGCTTTAGTGACATTATTTCAGTTACTTTTACAGGCTACTGATTGAACAGTTAATCATGT
TTCCTTCATGTTTGTGCTTGTTTTCTACCTTTCTGTATTTAGCCAATATCTGCCTCAGCTGCTTACATTTTGAGTGAAATATGTCGAGATAAAA
ATGATGCTGTTTTGCCCCTTGTACGACTGCTGCTGCACCATGATAAACTTGTTCCTTTTGCCACTGCTGTGGCTGAATTAGACTTGAAGGATAC
ACAGTAAGAGTTATATTTTATTAAATGTTAATTACATGTTGTTGAAATTGGACCTTAGTTTGACAGATTGATTTTGTAGTCTGAATTTTTTTGT
ATTGATCAGCCTTACTGTATAGTAGGCTGAAAAGAAGTACTTAACAAATAACAAAAGGCAGTTAAGCTATCAAACAGTATTCACCTACCTTAAG
GACAACAATTAATGAAGGATGTTTTTCTTTAACTCAGGAAATTTCATTTGTTACTAATCTGCTTCTTACTGTAATGGTTCACGTGTCACAAGTT
AAAACACCAGAGCAGAAGTGTTTTTTTCTTTATTCATTATGTATCACCCTTATATGCATATGTATGACATTCTAAAAAGCAGTCAAGTGACTTC
TCATTCACGTCATAGTCTAACACTGTTGTTTTTTTACACACAGAGACATACATATACTCCACATTTTTGGTTCATAAACATGATTTGCCTTTTT
AAAAAAACATATATATATATATATATATATATATATATATACACACACACACACACATATGTATTGAGTTTTTTGTTTGTTTGTTTTTAAGACA
CAGTCTCGCTTTTTCACCCAGGTTGGAGTGCAGTGGCATGATCTTGGCTCACTGCAGCTTCTGCCTCCTAGGTTCAAGCGATTCTCATGCCTCA
GCCTCCTGAGTAGCTGGGATTACAGGCACTCGCCACCACGCCCAGCTAATTTTTGTATTTTTGGTAGAGATGGGGTTTCACCATGTTGGCCAGG
CTAGTCTCACACTCCTGACTTCAAGCGATCCACCTGCTTCGGACCCCCAAAGTGATGGGATAACAGGCATGACCCATCACACCTGGCCATTATT
TTAATATATTTTTCGTAAGTTTGAAAAACCTAATGGCAGCAACCTTTTAAATAAATGCTAAAAATTACTAAAATTGTAGGTCACTTATGCCTTC
TAGTATGTCTTAATCTATTTCAGCTAAATGCTTCTTTCTTAAATTGGTCAAGTATGTTATATTATTGGTTTCAAAACCTTTGTTTACTACATTT
GGTTTCATCTATTTCTATTTCAGAGATCCAAACACAATTTTTAGAGGAAATTCCCTCGCTACCCGATGTCTGGATGAGATGATGAAAATAGTGG
GAGGGCACTACCTGAAAGTAACATTAAAACCTATTCTTGATGAGGTACAGAATATATCTCCAACAATGATAGTTTGTGGCCTTATGATAGTTGT
TCTTTTATCAAGTGATCTATTGATGGGAAGGATTTATTCATTCATGTGTTACTGAAAAAATAGACTGAACACTTTAGAAGCAAATTATTTACTT
CTCTGTCAATGGATTTTTACTCAAAAGGTACAGTATAGTATAATCTACATTATTTCTTTGTTATGGGATTTCTATTTTAGCTAGAGTTTCAAAT
ATATGACAAATACTTTTTGATATCATGTTTTAACAAAATTGTATATCTTATCCCTGCATAGGACATTTTACCCTTTGCTACACTTTAAATTTCT
ATAATTAACTTTTATTATCAGAAAAATAAGTTATTTTCATTTTGTCAGACTACTTAGAAAAAAATCATTAACTGAAAAATTTATTTTTCAGATA
TGTGACTCCTCAAAATCCTGTGAAATCCATCCTATTAAATTGAAAGAGGGAGATAATGTACAAAATAATAAGGTAAGTCCTTGTTATATTATTT
ATAATTGCATTAAAATTGGTCTTAATGGTGTACCTTTCTCCCACTTACATTATGATAAAGCCATATAGAGGGAACCAGTCCTGTTTATTCTTCA
CATAAGCCTTTATTATTAGCAGCACTTCCAGCTCCTAGGATTAGTTTTTAGAATCTTATTTCCTCAGTGGTTTCACTTTATTTTTGTTCTTGAT
TGTCTCTTTTTGGCTCTAGTAACAATACTGAACTTAAGCTATAGCTACTTCCAGCCCCCGTTCTAGCCATTCCTTTGGGCTCAAACTGATCAGG
CTAAGTGACACAGTGACTTTATCAGTCAGTAGTTTCAGTGGCCTCTCAAAAAACAATGATTGTGTTAGGGTCACAAATTTTTATTGCAGTGGAT
AATAGTGTCACAATCTCCTTGTGCTTGTGTATCCGACTTATTTTCTGATTTCCTTTTGGATCATTTTATTCCCTCTTGCTTCTTGTGAGGTTTA
TTCATAGATTCACCTCTTACGCCTAGCAAATCTAAATTTAATTTATTAAATTAAGTCA
HUMAN SEQUENCE—mRNA
ATGGCGGCGCCGGCGCCTGCTGCTGCGGCGGCTTCTTCCGAGGCGCCAGCGGCGAGTGCGACTGCAGAGCCCGAGGCCGGGGACCAGGACAGTC
GCGAGGTTCGAGTGTTGCACAGCCTGCGGGGCAAGATCTGTGAAGCAAAAAATTTATTGCCATATCTTGGACCCCACAAAATGAGAGATTGTTT
CTGTACCATAAATTTGGACCAGGAAGAAGTTTATCGTACCCAAGTTGTGGAAAAATCTTTAAGCCCATTTTTCAGTGAAGAATTTTACTTTGAG
ATTCCAAGAACTTTCCAGTATTTGTCTTTCTATGTTTATGATAAGAATGTTTTACAAAGAGATCTCCGTATAGGAAAAGTAGCCATCAAAAAAG
AAGACTTGTGTAATCACAGTGGCAAAGAAACTTGGTTTTCATTACAGCCTCTTGACTCCAATTCAGAGGTTCAGGGTAAAGTTCACCTTGAATT
AAAACTGAATGAACTCATAACGGAGAATGGAACTGTATGCCAGCAGCTTGTTGTACACATCAAGGCATGCCATGGGTTGCCTCTCATAAATGGC
CAAAGCTGTGACCCTTATGCAACAGTTTCTCTAGTGGGCCCTTCTAGGAATGACCAAAAGAAGACAAAAGTAAAGAAGAAAACAAGCAATCCGC
AGTTTAATGAAATCTTTTATTTTGAAGTAACCAGATCCAGTACTTACACCAGAAAGTCCCAGTTCCAGGTAGAAGAGGAGGACATTGAAAAGCT
AGAAATCAGGATCGACTTGTGGAACAATGGAAACCTAGTCCAAGATGTTTTCCTAGGTGAGATTAAGCTTCCTCTGAACCTATTAAGAACTCAT
TCCTCTCATCAAGCCTGGTACTTGCTACAGCCAAGAGACAATGGAAACAAGTCATCCAAAACTGATGACCTGGCGTCTCTTCGATTAAATATAT
GTTATACAGAAGACTACGTGCTTCCTTCAGAGTACTATGGTCCTTTGAAAACTTTGCTGCTAAAATCACCAGATGTTCAACCAATATCTGCCTC
AGCTGCTTACATTTTGAGTGAAATATGTCGAGATAAAAATGATGCTGTTTTGCCCCTTGTACCACTGCTGCTGCACCATGATAAACTTGTTCCT
TTTGCCACTGCTGTGGCTGAATTAGACTTGAAGGATACACAAGATGCAAACACAATTTTTAGAGGAAATTCCCTGGCTACCCGATGTCTGGATG
AGATGATGAAAATAGTGGGAGGGCACTACCTGAAAGTAACATTAAAACCTATTCTTCATGAGATATGTGACTCCTCAAAATCCTGTGAAATCGA
TCCTATTAAATTGAAAGAGGGAGATAATGTAGAAAATAATAAGGAGAATCTGCGCTACTATGTAGACAAGTTATTCAATACAATTGTAAAATCA
AGTATGAGCTGCCCCACTGTAATGTGTGATATCTTTTATTCTCTAAGGCAGATGGCTACTCAGAGATTTCCTAATGACCCTCATGTTCAGTATT
CTGCAGTGAGCAGCTTTGTATTTCTTCGTTTCTTTCCTGTAGCCGTAGTATCACCTCATACTTTTCATTTGCGACCTCATCATCCAGATGCACA
GACAATTAGAACATTAACTCTCATCTCAAAAACTATACAAACTTTGGGAAGCTGGGGGAGTCTGTCCAAAAGCAAGTCAAGTTTCAAAGAGACA
TTCATGTGTGAATTTTTCAAAATGTTTCAAGAAGAAGGATATATTATAGCAGTTAAAAAGTTCTTGGATGAAATTTCATCTACTGAAACTAAAG
AGTCCAGTGGTACGAGTGAGCCTGTGCACCTGAAAGAAGGTGAGATGTATAAAAGAGCTCAAGGAAGAACTCGGATTGGAAAAAAGAATTTTAA
GAAACGATGGTTCTGCTTAACAAGCAGAGAGCTCACCTACCACAAACAGCCAGGCAAAGATGCAATCTACACAATCCCAGTAAAAAACATTCTT
GCTGTGGAAAAACTGGAAGAGAGCTCTTTCAACAAGAAAAATATGTTCCAAGTAATACATACGGAGAAACCACTCTATGTCCAGGCAAATAACT
GTGTAGAAGCTAATGAATGGATAGACGTACTCTGCAGGGTGAGCCGATGCAATCAAAACAGGCTCAGTTTTTATCATCCCTCTGTGTATCTAAA
CGGAAATTGGCTCTGCTGTCAGGAGACTGGTGAAAACACTCTCGGCTGCAAGCCATGTACTGCAGGTGTCCCTGCAGACATCCAAATAGATATT
GATGAAGACAGAGAAACAGAAAGAATTTATTCCCTTTTTACCCTCAGTTTACTTAAGCTGCAGAAGATGGAAGAGGCTTGTGGAACTATTGCAG
TCTATCAAGGACCACAGAAAGAGCCTGATGATTATTCTAACTTTGTAATCGAGGATTCTGTAACAACCTTTAAGACAATTCAGCAAATAAAAAG
CATAATTGAGAAGCTGGATGAACCTCATGAAAAATATAGGAAGAAAAGATCCAGTAGTGCAAAATATGGGAGCAAGGAAAATCCAATTGTTGGG
AAAGCATCTTAGAGTTTAACAGATTGGTTCAGAAGAACTGGAAAATATTATTTT
HUMAN SEQUENCE—CODING
ATGGCGGCGGCGGCGCCTGCTGCTGCGGCGGCTTCTTCCGAGGCGCCAGCGGCGAGTGCGACTGCAGAGCCCGAGGCCGGGGACCAGGACAGTC
GCGAGGTTCGAGTGTTGCAGAGCCTGCGGGGCAAGATCTGTGAAGCAAAAAATTTATTGCCATATCTTGGACCCCACAAAATGAGAGATTGTTT
CTGTACCATAAATTTGGACCAGGAAGAAGTTTATCGTACCCAAGTTGTGGAAAAATCTTTAAGCCCATTTTTCAGTGAAGAATTTTACTTTGAG
ATTCCAAGAACTTTCCAGTATTTGTCTTTCTATGTTTATCATAAGAATGTTTTACAAAGAGATCTCCGTATAGGAAAAGTAGCCATCAAAAAAG
AAGACTTGTGTAATCACAGTGGCAAAGAAACTTGGTTTTCATTACAGCCTGTTGACTCCAATTCAGAGGTTCAGGGTAAAGTTCACCTTGAATT
AAAACTGAATGAACTGATAACGGAGAATGGAACTGTATGCCAGCAGCTTGTTGTACACATCAAGGCATGCCATGGGTTGCCTCTCATAAATGGC
CAAAGCTGTGACCCTTATGCAACAGTTTCTCTAGTGGGCCCTTCTAGGAATGACCAAAAGAAGACAAAAGTAAAGAAGAAAACAAGCAATCCGC
AGTTTAATGAAATCTTTTATTTTGAAGTAACCAGATCCAGTAGTTACACCAGAAAGTCCCAGTTCCAGGTAGAAGAGGAGGACATTGAAAAGCT
AGAAATCAGGATCGACTTGTGGAACAATGGAAACCTAGTCCAAGATGTTTTCCTAGGTGAGATTAAGGTTCCTGTGAACGTATTAAGAACTGAT
TCCTCTCATCAAGCCTGGTACTTGCTACAGCCAAGAGACAATGGAAACAAGTCATCCAAAACTGATGACCTGGGGTCTCTTCGATTAAATATAT
GTTATACAGAAGACTACGTGCTTCCTTCAGAGTACTATGGTCCTTTGAAAACTTTGCTGCTAAAATCACCAGATGTTCAACCAATATCTGCCTC
AGCTGCTTACATTTTGAGTGAAATATGTCGAGATAAAAATGATGCTGTTTTGCCCCTTGTACGACTGCTGCTGCACCATGATAAACTTGTTCCT
TTTGCCACTGCTGTGGCTGAATTAGACTTGAAGGATACACAAGATGCAAACACAATTTTTAGAGGAAATTCCCTGGCTACCCGATGTCTGGATG
AGATGATGAAAATAGTGGGAGGGCACTACCTGAAAGTAACATTAAAACCTATTCTTGATGAGATATGTGACTCCTCAAAATCCTGTGAAATCGA
TCCTATTAAATTGAAAGAGGGAGATAATGTAGAAAATAATAAGGAGAATCTGCGCTACTATGTAGACAAGTTATTCAATACAATTGTAAAATCA
AGTATGAGCTGCCCCACTGTAATGTGTGATATCTTTTATTCTCTAAGGCAGATGCCTACTCAGAGATTTCCTAATGACCCTCATGTTCAGTATT
CTGCAGTGAGCAGCTTTGTATTTCTTCGTTTCTTTGCTGTAGCCGTAGTATCACCTCATACTTTTCATTTGCGACCTCATCATCCAGATGCACA
GACAATTAGAACATTAACTCTCATCTCAAAAACTATACAAACTTTGGGAAGCTGGGGGAGTCTGTCCAAAAGCAAGTCAAGTTTCAAAGAGACA
TTCATGTGTGAATTTTTCAAAATGTTTCAAGAAGAAGGATATATTATAGCAGTTAAAAAGTTCTTGGATGAAATTTCATCTACTGAAACTAAAG
AGTCCAGTGGTACGAGTGAGCCTGTGCACCTGAAAGAAGGTGAGATGTATAAAAGAGCTCAAGGAAGAACTCGGATTGGAAAAAAGAATTTTAA
GAAACGATGGTTCTGCTTAACAAGCAGAGAGCTCACCTACCACAAACAGCCAGGCAAAGATGCAATCTACACAATCCCAGTAAAAAACATTCTT
GCTGTGGAAAAACTGGAAGAGAGCTCTTTCAACAAGAAAAATATGTTCCAAGTAATACATACGGAGAAACCACTCTATGTCCAGGCAAATAACT
GTGTAGAAGCTAATGAATGGATAGACGTACTCTGCAGGGTGAGCCGATGCAATCAAAACAGGCTCAGTTTTTATCATCCCTCTGTGTATCTAAA
CGGAAATTGGCTCTGCTGTCAGGAGACTGGTGAAAACACTCTCGGCTGCAAGCCATGTACTGCAGGTGTCCCTGCAGACATCCAAATAGATATT
GATGAAGACAGAGAAACAGAAAGAATTTATTCCCTTTTTACCCTCAGTTTACTTAAGCTGCAGAAGATGGAAGAGGCTTGTGGAACTATTGCAG
TCTATCAAGGACCACAGAAAGAGCCTGATGATTATTCTAACTTTGTAATCGAGGATTCTGTAACAACCTTTAAGACAATTCAGCAAATAAAAAG
CATAATTGAGAAGCTGGATGAACCTCATGAAAAATATAGGAAGAAAAGATCCAGTAGTGCAAAATATGGGAGCAAGGAAAATCCAATTGTTGGG
AAAGCATCTTAG

TABLE 13
MOUSE NOMENCLATURE
ICSGNM Gata1
Celera mCG3964
HUMAN NOMENCLATURE
HGNC GATA1
Celera hCT10890
MOUSE SEQUENCE—GENOMIC
TGCCTATAGTTTCAGAGGCTGAGTCCATTATCATTGTGGAAGCATAGCAGAAGGCAGACAGGAGTTGGGGCACTATCTGAGACCTTSATATCCT
GATCCATAAGGCAAAAAGAGACACATGAGCTTTTGAAGCTTCAAAGCCCAGCCCAGCCCCAGTTGACACAGGTCCTCCAACAAGGCTACTCCTC
CTAATCCTTCTCAAATAGTGCCCTACCCCTTGATGGCTAAGTATTCAAATAGATGAGCCTATGGGCCATTCTTTTTTTTTTTTTTTTTTTTTTT
TTTTAGTTTTCAAGACAGGGTTTCTCTGTATAGCCCTGGCTGTCCTAGAACTCAAACTGTAGGTAGACCAGGCTGGCCTGAATTCAGGAATCAG
CCTGCCTCTGCCTCCTGAGTACTGGGATTAAAGGTGTGCACCACCACTCCCTGGCTGGGCCATTGTTATTCAAACCACCGTGTGTGTGTGTGTG
TGTGTGTGTGTGTGTGTGTGNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNCTGGGTGCAGTGCTGGATATCTACAGGCCCAGTCTCAGTACTGAGGCTGAAGCAGGAAAGGCTGCACAGTTGAGGTCAGCCTGGTATACA
GCCGAAACCCTCGTCTCAAAAAACTAACATGATTTTCTGTTTTGTCAAGATAATTGCTTGTTTCAGGTTGCTCTGCCTGATTAGTTTTCTTGTG
TTTGTGGTGGGTTTTGGTATTTGTTTGTTTATTTGTTTTTGAGACCAGGTTTCTCTGTGTAGCCCCAACAATCTTGGAACTCTATAGATGGGCC
TTGGCCTTGAACTCACACGGACCTGCCACCTCCGCCTCTGCCTGGAATCAAAAGGTGGGAATTAAAAGCGTCCACCCTCACCTCCTGACACTGT
TGTTTTGAGAGCTTCCCACTTTAGCCTGGCCTTGAACTCAAACTAATCTTCTTGTCTCAGCCTCCCAAATACCAGGATAACAGGCATGAGCCAG
GGTGCCTGATTTCCGATGCTTTCCTTGTGCATACTCATGTGTATTCAGATGCCCGTGAACGTGTCAGTGTGTTCACATGGAAGGCCAAAACTGA
TGTTGAATGTCTTCGCCAATCATTTTCCACTTTACTGAGGCAGGTTCTCAGGGAACCTGGAGCCTGTCGATTTGGACTAGTCTCACCTGCCACC
CTGTTCCAGGGATTCTGTCCCCATCCTTCTACTTGCTGGCATGATAGGTAGACAATATGTCTGTACCACTAPGCTCTTGATGACTACCGAGTGG
AGTCTTATGAAGTCAAGGTTATGGGGTQTGGTGGTACACCCTGTTAAGTCCCACATCAAAGCAGCTCACAATACCTCTGCAGCCTGCGTTCAAG
GGGATCTAGTTCTCACTTCTGGCCTACACACACAGACACACAGACACACGGACACACAGGCACACAGACACACTGACACACTGACACACAGGCA
CACAAAGAAAAAACTTATCTTTTAAAACCAAAGTCAGTTGAGTGCAGTGGCATGTGCCTTTAGTCCCAGTGCTTGGGAGGCAGAGGCACAGAGA
ATCTCTGTGACTTCAAGGCTAGTCTGGTCTACTTAGTAAGCTCCAGGACAGCCATGGCTACACAGAGAGCTCCTGTCTCCAAAATGTTAATTAA
TTAAAAAAATAAAACTAAAGTAAAACATTTATTCAAGTACATAATTATTGTAAAGTTCTGAGACTAATTCCTTTACAACTATGGTTAAGTAAAC
AACTACTGACTTAGGTGTTCTGGGATATTTGAAATTCTACATATCTGTTGCTACACATATATACACGCACATACACATCTTGAGGCTAAATTAG
GATATGTGTCTAAGTTTTTCCAAGTGTTAGGCAAAAGTTTGTGTGTTAAAAGTGTGTAGGTTTAATACATTAAGGCAGTCAATAATATTCTCTA
AACATGATCCATTGTGTGAAAACCTTAAACACATCATTACTACCATCTGACGAATTTCTGCTGTATATTTAGTAAAATTCACGATTATCGGCTT
GTTTTATTTTTAATTATGTGTCCATACTTAATTATTTGAGTATATATATATATATATATATATATATATATATATATATATATAPATATATAPC
TGTGTAGGAGCATTGTAGACATGAGTACAGGCCCTTGGAAGCCAGATCCCACAGCTAAACCATCTCTCCAGCTCCTTGTTTTGTTTTATGAGAC
AGGGTTGTGTTACATAGTACTCCCTCATCCAGAACTTGCTATGTAGATCAAGCTTGACTAGAACTCACCACATAGTCAAAACTGGCCTTGAACT
CCTGGTTCTCCTGCCTCTACCTCCAATATACCATCATCCCCCAGTCAGGTGCCATTTGTATACTTCTCACACTTAAAATCTGGCCTGCAGCAAG
ATGTGGCAGCTTATATCCATGAGTCAAGCACACGCACCCCCAACTCTGACACGAGAGGATCACCAGAAGTTGCAGGCCAGCTTAGGCTGGCCTG
TAGTGTATGGTTCTGTCTCAAAAGAGAAAAAAAAGGAGAAAAGGAGTAAAGAAAGTGTAAAAAGCTTTGGGCTAGGGGTACAGCTCAGTTGATA
GATCACTTGCCTAGCATGTGCAAGCCCATAGGTCTATTATTAAATCTGCGGCAATCACATCATGTACCCCAACTCAGGTGTCGCCATCTGTTGT
CAATAAGCTATTTAAAAGAGGCAAATTAAAAGTGAAAAGCAGAGCCGGGCGTGGTGGCGCACGCCTTTAATCCCAGCACTCTGGAGGCAGAGGC
AGGCGGATTTCTGAGTTTGAGGCCAGCCTGGTCTGCAAAGTGAGTTCCAGACAGCCAGGCCTATACAGAGAAACCCTGTCTCGAAAATAACAAA
ACAAAACAAAACAAAACAAAACCCAAAAAGTGAAAAGCAAAAAAAAGTGATTTAGTCAATCAGGCCACATTGGGAAGAGGGATAAAGACCTCAA
TACAAGTGCTTCCTTCGAATAGCTTCAATTCTACCAGACCAGTCCCAATTCTACCAGACCAGTCCCAATTCTGAACTGGCACATGGTGTCTATG
TCTGAAACAAAAGGATAAATACAGTGACCGTAGGGATGGCAGCTTCAATATAGTGAGGGGTACCACACCTAAGAATCATTTTCCATGGCTCTTA
GTTACTGTTGGAAGGGGTGGGACAAGCAACTCCATTTCACTGGCTTGAGACAGGCTCCCATCAGGAGGTCTACACTGGCCTCAAATTTGGGATC
TGCTTGTCTCAGCTTCCCAGGCAAGTGCTACCATGCCTACTGTGACTTTATTTTCAGTCTAACCATCTCTCTACCCCCCATCTCTGAACAAATA
TTTTCTGAAGACATTGCTAATTGACCATCTGGTGGTTTGCTTTCCAAAGTCCCATGATGCCAGCCAAAGACTCCTTCTGGTTGGTCTGCTTTGA
ACCCCTTTGGCAGCGTAATGAGAGATGTCAGCAGCTACAACTAGTCAGCCTTTCAGGCCTGATTTAAACAACTGGGAATATAGAGGGATTGGCT
CCGAGGTCAGGTACACCTGAGGTCCATACACTTGTCCTGTGGCTAATGTCCTGCTGTCTCAATGATGCTGTTCGGGTTCATTTGTCAAAAGCCT
GACTTCTGTAGAACGTTAACAGACAGTTCACTTATAAGGCTCTGCAAAACAGAGTTAGTGGGCTGGAGAGATGGCTCAGTGGTTAAGAGCACTG
ACTGCTCTTCTGAAGGTCCCGAGTTCAAATCCCAGCAACCACATGGTGGCTCACAACCATCTGTAACAAGATCTGATGCCCTCTTCTGGAGTGT
CTGAAGACACCTAGAGTGTACTTACATATAATAAATTAATAAATCTTAAAAAAAAAAAACAGAGTTAGCAGGTATTTCGCAACCAGTAACTATA
ACACAGTACACAAACACTAGTTGGGGCTGGNNNNNNNNNNNNNNNNNNNNGAAACTGAGTCTGGGATGGTGAGACTCAAAGTTCCTGAAAACAG
ACCCTTGTCCCCACCCACCTGTTACTCTTCTCAGAGGAAGCCAGTAGCTTTGGCGTCATTTCAAAAGTTGGGCGGGGATGGCAGAGATAAGCAG
TGTGGGGGCAGGAGTACTGACCCCCCCCCCATAGAACAGGGAGGAACTAACGGGGCCCACAGTCTGCAGACACAACTGAAGTGGGCCAGCGGAT
AAAGCCGGAACCACGGGGTTTCTGAGAAAGTCAGATGGCAAGATACAACAGATAGGGATGAAGTTGGGGAGCACACAACGGGTCAACCCCAAAC
TCCTATGGGAGCTGTCAAGGGGTCCAAGGGATCTTGGTCCTGCATTCTGCCTCTGCAGAAGGAAAGAAAGGAAGGGGAAGAGAGAATAAAAAGA
GGAATGGAGGAGGCCTTGAAGAATGGGAGAAAGAGAAGCAAGGCTTGCCGGAGGAGGAGAAAGAGGAAGGTTAGAATAGGAAAGTAAAGGAGGA
AGGAAAGAGGAGACACAGAAAGAGGAAGAGAAATATGGTACATGAAAAGGAGGGACAGGAGCAAGCTGCGAGGGAAGGATGGACCAACAGAGAA
AGGAAGGGAATACAAGACAGTGGAAAGAGGAAAAGGGGAAGAAAAGAGGGAAGAAGGAAGACCTGGAGAGACCTGGAGATGAGGAAGATGGGAG
GAGGGATAGGAGAGGAAGAAGAGGGACTCAGAGGCAGCAAACTGATGGAGTAGTACAGGTATGACAGACAAGAGGCAACAGGAGGATGGGGAGA
GATCAGAGGAAGATCGGAGAAAGGGACAAAGGATGATGGAAGAAGACAAGAGACATGACAGCCTAGGCAGCCATGGAAGGGAAGGGGAGAAAAA
TAATCTGAGAGCCCAAAAGGAGACACACAGAGTCTCAGTGACAGATTCGGAGAAAGAGAAAAAAAAGGAGGGAAGAGCCAGGCGGTGGTGGCGC
ACGCCTTTAATCCCAGCACTTGGGAGGTGGAGGCAGGCAGATTTCTGAGTTTGAGGCCAGCCTGGTCTACAGAGTCAGTTCCAGGACAGCCAGC
GCTACACAGAGAAACCCTGTCTCGAAAAAAAAGAAAGAAAGAAAAAAGAAAAGAAAAGAAAAGAAAAGAAAAGAAAAGAAAGGAGGGAAGAGAA
TGTTTCTGTGTCTCACAACCCTTTCTGTCCTCACAGGTTCAACCCCAGTGTTCCCATGGATTTTCCTGGTCTAGCGGCCCTGGGGACCTCAGAA
CCCTTGCCCCAGTTTGTGGATTCTGCCCTGCTGTCCTCACCATCAGATTCCACAGGTTTCTTTTCCTCPGGTACCAGACATTTGGATGCAGCAT
CTTCTTCCACTTCCCCA~ATGCAGCCACGGCCGCAGCATCAGCACTGGCCTACTACAGAGAAGCTGAGGCCTACAGACACTCCCCAGGTAACTG
TTGGGTGGCTGCCTTGGCACTCGCTGGAAGCTGTCAGGGTTGCCATAGAGATGGTTAGCTAGTTCACAGTTCTGTCAGCTCATCTTGGAAAGAG
TTAGAGCTCTGTCGATAGGTGTACCGCCCCATTTGTACCAATCCTCGTCTCTGTCTGCCATACTAATAGTGAGCACACGGAGAAGCCAGGCTGG
AAGTGTGTACGAAGCTGTCTTATTTGTCACTTGACCTGCAGCCTCTTGGTCTCAATATCAAAAAGGGATGGAGGGGATCGTCCTTGCTATAGTG
ACCAGAGGGACATAGAACTCCGAAGTCACCCAAGCACCAAGAGACTATTGTAGGAAGACCAGAAAACTAGCCACCATGTTGGGACAGACGAACA
CCATTTCACTGTAAAAGTAGGACTACCTAAGGTTTGCTGTTCTTTCCTTTTCCTCTCTACCCCTTCAACAGTCTTTCAGGTGTATCCACTGCTC
AACAGTATGGAGGGAATTCCTGGGGGCTCACCTTATGCTAGCTGGGCCTATGGCAAGACGGCACTCTACCCTGCCTCAACTGTGTGCCCCAGCC
ATGAGGATGCCCCTTCCCAGGCCCTGGAAGACCAGAAACQGAAGAGCAACAACACGTTCTTGCACACCTTGAAGACGGAGCGGCTGAGTCCAGA
CCTCCTGACGCTGGGGACTGCACTGCCTGCATCGCTCCCTGTCACCGGCAGTGCTTACGGGGGAGCTGACTTTCCCAGTCCTTTCTTCTCTCCC
ACTCGGAGCCCTCTCAGCTCAGCAGCCTATTCTTCCCCCAAGTTTCATGGAAGCCTGCCATTGGCCCCTTGTCGTGAGGCTTTCAAAAAGGGAC
CAGGAGGTTGAGTTCGGCTAGGTGGCTCAAGACATAGCTAAGCTGACCGAGCCTTGCTTCCTTTTCTCTCCATCTACCAGAGGCCAGAGAGTGT
GTGAACTGTGGAGCAACGGCTACTCCACTGTGGCGGAGGGNNNNNNNNNNNNNNNNNNNNNNNNNNNNNNNNNNNNNNNNNNNNNNNNNNNNNN
NNNNNNNNNNNNNNNNNNNNCAGTGCTGGTGCATGCCTTTAATCCCACCACTTGGGTAGGCAGTAGGCACCTGGATTTCTGAGTTCAAGGCCAG
CCTGGTCTACAGAGTGAGCTCCAGGAACAGCCGAGGCTACACAGAGAACCCTGTCTCGAAAACCAAAAAAAATTTAAAAAAAGACTTATTTAAG
AACGCCACCTGGGCTGGTGAGATGGCTCAGTGGGTAACAGCACCCGACTGCTCTTCCGAGGTCCGGAGTTCAAATCCCAGCAGACCACATGGTG
GCTCACAACCATCCGTAACAAAATCTGACTCCCTCTTCTGGTGTGTCTGAAGACAGCTACAGTGTACTTACATATAATAAATAAATAAATCTTA
AAAAAAAAAAAAAAAAGAACGCCACCCAACTCATACTGGTCCCGGCACTATTATACTTGCCCTCTTCCTTATCCTTTCCCCATAGGGACCATCC
ATCCCTTTCCCATGCAGGAACCCCGCTACCTACCTAGAACTCCCATCCAAGGCCAGGAATTTTTACAGCTTCCCCAAGGGATTCTTGTGTTTGT
TTTGTTTTGTTGTCTGATTAATTGATTGATAGATTTGGTTTGTTTTTTGTTTGAAATGGGGCTTTCCTATATAGCCCTGGGTGGCCTGGAGCTA
TCTATACGATACTGGTTTTTAATTTGCTATGGTCCTCCTCTCTCTGTCTCTTGAGTGCCAGGGTTACACCCCTGTCTTCCTCCAAGACATTCTG
ATATGTGGTCAAAGTCTAAAACTAGAGATACAAGAAGTCTTGCATAGCTATAGAAAAGAAACTCCTTGCTGGGCAGTCGTAATGCATGCCTTTA
ATCCCAGAACTGGAGACAGAGCCACGTGGATCTCTTGTGAGCTCAAGACCAGCCTGGTCTACAGAAAAAATTCCAGGAGCCAAGGGCTACACAG
TGAAGCCTGTCTTGACAGATAAATACACACATACATAGATACATAGATCAATAAACTTCTTATCCCAGATAGGAACCCTTTCCTTAAATAGCAC
TCTCATCCCTTAAGCTCTTTTCTCAGGCCCTGTAAGGCCCTGTCCCCGAATCCTACCTCTCCCCTACCCCCAGCACTAATCTCCTGTCCTTTTA
TTCCTCAGATTGTCAGCAAACGGGCAGGCACCCAATGCACTAACTGTCAAACGACCACTACAACACTCTGGCGGAGGAATGCCAGCGGAGATCC
GGTATGCAATGCCTCCGGCCTCTATTTCAAGCTCCATCAGGTATGCTATCCTCCATGTTGCTTCCTGTCCTCCAAGCACACTCCCGGCTCCTCA
TCCAGCCCTCATCTACCTTGAGCTGTCTTCCTCTGCCTTTGCTTCCTCCCTCTTTGCTCCTCTTTCTCTTCTCAGAAGTTGTGTACTTGCGCCT
CCTTGCCCAATTTCCACGGCTTCCACCTCAAGCCTCCTTCCATCTGTCCTCTCTTTCCTCCTTGCCACCTCCCGTGATTTGAGTCTTGGCTACC
TCTTACCAGTGTAGGAGGATATTCTGAGCTGAGATCAGCGCCACACACAAGCACAGGTCTCACCTCCCTAGCACACCCTGGGTATATTACTCTA
GCCATTGAACTAATTCCCCATTCTTAACAAAGTGGTGTTAAGAGACTGTCCCTAGCCCAGCTACATCAGCACAAAGGGCTCGAGCTCGTAACCC
CTGTCGTGGTCTCCGTTTCTACAGGTGAACCGCCCACTGACCATGAGGAAAGATCGAATCCAGACGAGGAACCGCAAGGCATCTGGCAAAGGGA
AAAAGAAGCGGGGCTCGAATCTGGCTGGAGCGGGAGCGGCTCAAGGACCAGCTGGTGGCTTCATGGTGGTAGCTGCTAGCAGCAGTAGTGGGAA
TTGTGGGGGAGGTCGCCWCAGGCTTGGCACTGGGCACTGCAGGTACTGCCCACCTCTATCAAGGCCTAAGACCTGTGGTACTGTNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNTACTGGATGAGGTT
GCAGGTATAATATGTCACAACTGAAACTCATGATGCACCAACCGAACTCTGTATCTGAAGCCTTGTGGACGGCATCCAGGTCGCCCCATATCTC
ATCGTAAGATGCTGGATGACTGATTACTTGTGGATGAGGTGGCGGGAGATGCCACCGTCTCATCAAGTTGCAGGTAAAACAGCATGTGGAGCTG
TGCGTGCAATAACAGGTCACCAGACATCGAAGGCTGTAAAGGTGTGCTTGTGCATGACTGCCCTATCGTTGACACAGTCTGACGACACATAAAC
TGGACATTTTTGAAACACTGAGCAAGAACTCAAGTGTAGGATTGTCCTGAAATCAAGGCCATTGAGGCCTACAAAGCAAATGATATACATATAG
TGGAGAGAGAGAGAGAGAGAGAGAGAGAGAGAGAGAGAGAGAGAGGATCTCAAAAATATAAATTAATCACAATAGTTTATATTCCTTTCAGAGA
CTGTCCACAATCGCCATGGCCCTATAAAAGTCCCCAAAGCAGCTCAGCCCTCAGCTCCCACACCTTGCAGACACCCCTTACCTGAATAGCAAGT
CTACTCAGAATTGCTCTTACACAGGWTCCTGGCCTTACCTTGAAAATAGAACCCAGGTTCACACCCTCATACAAACAGCTGCCCTGACTGGTGC
CGGAAGCACAGGCTAGCCCTGAGATCCTATGGAACAAGACCTCTTGCTCACTCTTGTAAACTCCCTCTCCTTCCGTCCTTCTCTCTTCCTCTCT
CTCCCCCTCCCTCCCTTTCTTCCTCTCCCTCTGTCCCTCCCTCTCCCAGCCTTTCCTTATCTCCCTGCTCTCACTCTCTCCTTTCCTCTCATGC
ACCTCAGGTCTCATACCTGCCGTGCAGTTCAAGCTTCCCTCTAGTTGATTTGTTTTTCTCGATTTTTCTGAGTGGGGGTCTCACTCTGTATGTG
ACCCAGGCTGGCTTATCTGCTCAGTAAGTATCCCAAATTCTGGCCTTGGACTCATGGCAATCCTCCTGCCTCAGCTAGGATTTCAGGCAAGAGC
CACTGCACCTCGAAGGGAGCCCTTTCTCCAATAATTCACAGGCAGCCCATCCTGACCTGGATACGACCCGCAQTCCCTACCAGCCCATTCAGCA
ATNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNCCCTGTCTCAAAAACAAACAAAACAAAAAACCCAAACCAAAAACAAACAAACAAACAAACAAAGCACAGCTGAGG
TACATCAGTTAGTAAAGTGCTTGCCTTACAAAGACCAGGACCTCAATTTGACTCCCAGAACTTTGGTAATACAGGTCATCTTGCGGGAGCATAC
TTATAATCCCAGTACTCATCCAAACTGGCAAACCCCTGGGATTTCCTGGCAAACCCACTTAGCCTATTTGAGGAATCCCCAGCCAGTGAGAGAA
ACTATCATGAAAATGTGCTCTGGGGCTGGTGAGATGGTTCAGCGCATAGGAGCACTTGACTGCTCTTCCCAAGCTCCTGAGTTCAAATCCCAGC
AACCACATGGTGGCTCACAACCACCCATAATGAGATCTGATCCCCTCTTCTGGCACATCTGAAGACAGCTGCAATGTACTTATGCATAATAGTA
AATAAATCTTAGAAAAAAAAAAGAAAATGTGCTGTGTCTCACAAACAACATCTGAAGTGGTGCTCTCGACTTTACATACATGTCAATTCTCCTC
TACACACAGTAACACACATACACAAACCCACTCACATGCATGTCAGGAAAAAGACGACACATAAGCCACACTCTGAATTCCATAAACGCCTGTC
TGGACCCCGCATTAGACCCTTCAGCTTGAATATACAAGATTTCACAGATCTGTCTACTCCAGGAACTTGGGTCCAATGCTGTACACATACACCT
AGATTTTTAAAAAATTATTGTTTGATGAATCTTTGGGATGTTTTTATACTAGCCTGTCCCTGTGGGTTGTTAGGATCCCTGCGTTGTGAACTAT
TTTATGTTGGGCACAAATGTTTTTAAGTGCAGAAGAAGTTAAAGCAAGAGCATTGTTTGTCTTGTGGCACCACCGATGGTCCACAGAGGCAAAT
GTCAGTGTGCAATCGGATGTTTTCACAAGCACCCCCTGTGTGAGTGGAGGCATAACAACTGGGAACAGATATAAACAGATkGCATGCACAAAGG
TCATTCTCCCAAAAGAGGAGGCACAAGGAATATCCATCTGGCAAAGGGCATTCGTCCATAACCCTCTGGGCTAAAGTTGTCATATGTGATACCT
GACTTATTATATAAGTTTCTGGCTTGACCAACTACTGAAAGCCACTCTTCTGGCTTTAATATAATCTTATATCTTAGTCTGTATAGAAAAACAG
CGCCTTCACATCCTTCCCTCTTCCTCAATCATGTCTCATAACGTTTGTTTGTTTGTTTGTTAGTTTGTTTTGTTTTTCTCTAGACAAGGTTTTT
CTCTGTAACAGCCCTGGCTGTCCTGGAACPCACTCTGTAGACCAGGATGGCCTTGAACTCAGAATCTGCCTGCCTCTGCCTCCCTAGGTACTGG
GATTAAAGGCATCCCCCCCCCCCCCACCCCCCAGAATGTCTCTAMTTACCAGCTAAATTATAATCATTCTCCTGAGACCAGACAAGTACACATA
GAGAAATTCTTACCTTTGCAGTAAAAGAATTCCTGTGCCAGGCTTGGGGCTGTAGCTCAGTGGCAAAGCACTTGCCTAGTGTGTATGAGTGCCT
GGGTCCCATCCCCATGCCTGCATAAAAGAAAACATGTCAGTCACTGTGATTTGAAATCAATACAACTATTTTTCCAGACTTATTTTGAATTATA
TCCAGAAGTGCCGTCCACCGCTTCTAGGGCCCTCCCACAAGAGCTTCTCACCGGAGCCTCCACTTTCTGCTTCCCTACCCACGAACTTCGTAAC
AAGAACTCCAAGTACCCGGAGGAAAGTAGTCAGAAAATGAATTACCTGGGTCCCTGAATTACCAACAGTCAGTATTCTGATTGGGGCTTATATG
AGAAAGAAGAAATATTTACTTGTGCTGGCTGGGTTTTCCCCATGATTTATTTATTTTTACTCACATTACGTCCCCATCTGCAGCACACCCTCTT
GTTCTTGGTGGTCATCCCATGGTACTGGCATCTCCAAAACTGCTGGGGTCTTGCACTACAATTGGGCTGCACTTTCACCAATAGGTTCTGTTCA
CGGTGCCAAGCCTCAACTTCTCTCCATGACCCCATCAATCTTGGAGCTTCATTTGCTATGGAGGCTCCACTTTCAGCAATGGCCTCTCCTGGCT
CTCACAGTGCTAGGCGTCAGCTGCTCTCCATGAACTCTCTACGCCTTCAAACCAGTACCACCTGGGTGACTCTTACACTGCCAAGTTCAGCTGA
TAGCCCAAGGTACAAACTTGCCTGCCTCTCGAACATAGCTTTTAGATTTCACCTTGACACTGCTGCTCCTCTCCTCTACCTCCTCCTCCTCCTC
CTCCTCCCCCTGCTTTTCTTTCTCTCTTCCTTCCTTTTTCTTTTTCTTTTGCCCCGTCTGTCCTGGAACTCACTCTGTAGACCAGGTTGGCCTC
AAACTCAAGAGATCCACCTGCTTCTGCCTCCCTATTGCTGGGATCAAAGGCATGGACCACTTCCACCTGGCATTAAGATTTTTTATTTTTGTGG
ATCTGTGTAAGTACACTATAGCTGTTTTCAGACACACCAGAAGAGGGCATACGATCTCTATAGATGGTTGTGAGCTACCATGTGGTTGCTTGGA
TTTGAACTCAGGACCTCTGGAAGAGCAGTCAGTGCTCTTAACAGCTGAGCCATCTCTCCAGCCCTCTGGTCTCTTCTAAATCACCATTAATTTC
TCATCTACAGATGAGCAGAACTGAATATCCCAGCAAGACAAAGGATTCACTTAAGCAGTTGTGCTAGCTTGTTAAATCTTCAGCCCCTACTGAC
CAGAACCATGGAATATTAATTCAAAACAACAAATGGCCCCAGTAGTGTCTTTAAACTTCTCCCTGTCACTTCACAAGCCAGGTCTCTATCCTCG
GCATTGCTCTCAAGAGTCTCATCTTCCAACCTTTTACAGAACAGCTCATCAAGCAATGAACACCCTCCCCAAAACAACATCGCCAGGGTTGTCA
CAGCAATGCCCCGCTCCTAACACCAACTTGTCTGAGTTAAGGTTACTATCTCTGGGATGGAACACCATGACCAAAAGCAACGTGCAGAGGAAAG
GGCTCATTTCACTCACAGTTCCACGTCATAATAGGTAATCATCAAAACCAGTCAGGACAGGAACTCCTACGGCAGaAACCTGGAGCCAGGAGCA
GATACAGAGACCATGGAGGGTGCTAATTACTGGATTGTTCCTCATGGCTTGCTCAGCTGCTTTCTGAAACAATCCAGGACCACCAGCCCAGGAG
TGGCGCCACCCACAATGAGCTGGGCCCTCCTCCTCAATCACTAATTAAGAAAACGCCTCAGGAGAGGCCTGCAAACCCCAGGCCACAACAATAG
ACCATCGCCCTTCCCACTATCAGAGAGGCTCTATTTAGCACCTGATGGGAGCAGATACGGAGACCTGCAGCGAAAAATTAGGCACCTCTGGAAA
CCCCAGCGAAGAGGGGGAGGACAAGTTGTAGACGTCACAGCGATCAAGGATTCCAGCAGGACACTGCCCACGGAATCAGTGAAGCAGGGCTCAT
AGGGGCTCAAAGACACCGAACCAACAACCAGGAGCCTGCATGGAGTTTGCTAGACTCTCTGCATATGCTACGGTTGTATAGCTTGGTGTTCTTT
CTCGACTCCTAACAATGGGAGCAGGGGCTGTCTCTGACTTTTTTGCCTGCTTCAGGGACCCTTTTCCTCCTACCGACTGGCCTGGTCCAGCCTT
CATGTGAGGCTCTGTGGCTAGTCTTACCCCATCTTGTTATTGCCGTGTTTAGCAGGTATCCGTGGAAGGCCTGTTGTCTTCTGAAGGGAAAATG
CAGGAGAAGTGGATCTCGGAGAGAGGGGATTGGGAAGGGGTGGAGGGAGGGCAAACTGCTTTTGAGATGTAATGTAGGAGAGAAGAAGAAAAAA
GATTGATACAAATAACTGTCCTCTAAGTACCCAAGAATAGAAACATAACCATTTAGAGAGGGGCAAAGAAAGCCTTACAGGCCTGCCTACACCC
TGATCTTACGGAGGCATTTTCTTTGCTTTTTATAATTTTGCTAGTTATGATACATAATCTAATATTTTTAGAAGAGAGAGGGTTGTCAAGGGGG
TCATAACCCGCAGGTTGAGAAGTACTGTTTTAAGTTGCTGAACTAGAGAGATAGATGCCTTCAGTGCTTGAGTCCCAGCACCCACATCTAAAGG
CTCACAAACACTTCTCACTCCACCTCCTGGAGGATCCAGTGTCCTTTCTTGCCTCCTTAGGCATACACACAGGCAAAATCCATGAAGTATGCTG
GGCATGGTGGCACATGCCTTTAATCCAGCACTTGGGAGGCGGAGGCAGAGGCAGAGGCAGAGGCAGAGGCAGAGGCAGAGACACATGCATCTCC
GTAAGTTGGAGGCCAGTCTGGTCTACATAGTTAGTTCCAGTACAGGTGGGACTGCATGGAGAGACCCTGTCTCAACAAATAAGTAAACAAGTAG
CAGAGGAAGATGGCGGCCGCCTTAGCTGTTCCTGGAGCGGGATCTGCAGTGGTCTTTGGGCCTGAGGCTCTCACCCCAGACTGGGAAAACTGAG
AAGTCTCCACACGGACCATGATCATGGCTTTGTAATTTAATGGAGGCGTGGTTCTAGGAGCAGACTCCAGAATAACACTGGGTTTTACATCGCC
AATCGACTGACTCACAAGCTCACCCCTATTTATGATTACATCTGCTGCTGCTCAGGCTCAGCAGCTGATACCGGAGCAGTAGCAGATGCTGTCA
GTTACCAGCTTGGTTTCCACAGTATTGAACTGAACAATTCTCCACTAGTACACATGGCTGCCAGGCTCTTTAAGGAGATGTGTTACCGGTGCAG
AGAAGATCTGATGGCAGGAATCATCATTGTAGGCTGGGACCCTCAAGAAGGAGGGCAGGTGTCCTCTGTACCCATCAAGCGTATGATGGTAAGA
CAATCTTTTGCCACCGGAGCCTCCGGAACCTCATACATCTATGACTATGTTGCTGCTATCCATCCTGAAGGCACGACCAAGGACGAATCTCTGC
AGTTCACTGCCAATGCTCTCTCTTTGGCTAGAAAACACGAAGGCTCCAGTGGAGGAGTTATCCGCTTGGCAACCATTCAAGGATCAGGGGTAGA
GCGGCAGGTGCTTTGAGGAGACCAGATCCCCAAGTTTACCACTGCCACTTTGTCACCTCCCTGAAGCCTAGATGCAAAAGCTAATAAACAGTGT
TAAAGAAAAAAATAAGTAAACAAATATAATTTTTAAAACAAATGGTTGGCTCCGTGGTCGTCGCGCATGCCTTTAACCCCAGCACTTGGGAGGC
AGAGGCAGGCAGATTTCTGAGTTCGAGGCCAGCCTGGTCTACAAAGTGAGTTCCAGGACAGCCAGGGATATACAGAAAAACCCTGTCTCAAAAA
CAAACAAACAAACAACAAAAAATACCAAACAAACAAACAAAAAACCCCAAATGGTTGTTAAGCTGGGCAGTGCTGGCACATTCCTTTAATCCCA
GCACTTGAAAGGCAAAGGCAGGTGAATTTTTGAGTTCGAGGCCAGCC~GGTTTATAAAGTGAGTTCCACGACAGCCAGGGATATACAAAGAAAC
CCTGTTTCAAAAAAAAAAAAAAAAA
MOUSE SEQUENCE—mRNA
ACTCGTCATACCACTAAGGTGGCTGAATCCTCTGCATCAACAAGCCCAGGTTCAACCCCAGTGTTCCCATGGATTTTCCTGGTCTAGGGGCCCT
GGGGACCTCAGAACCCTTGCCCCAGTTTGTGGATTCTGCCCTGGTGTCCTCACCATCAGATTCCACAGCTTTCTTTTCCTCTGGGCCAGAGGGT
TTGGATGCAGCATCTTCTTCCACTTCCCCAAATGCAGCCACGGCCGCAGCATCAGCACTGGCCTACTACAGAGAAGCTGAGGCCTACAGACACT
CCCCAGTCTTTCAGGTGTATCCACTGCTCAACAGTATGGAGGGAATTCCTGGGGGCTCACCTTATGCTAGCTGGGCCTATGGCAAGACGGCACT
CTACCCTGCCTCAACTGTGTGCCCCAGCCATCAGGATGCCCCTTCCCAGGCCCTGGAAGACCAGGAAGGGAAGAGCAACAACACGTTCTTGGAC
ACCTTCAAGACCCAGCGGCTGAGTCCAGACCTCCTGACGCTGGGGACTGCACTGCCTGCATCGCTCCCTGTCACCGGCAGTGCTTACGGGGGAG
CTGACTTTCCCAGTCCTTTCTTCTCTCCCACTGGGAGCCCTCTCAGCTCAGCAGCCTATTCTTCCCCCAAGTTTCATGGAAGCCTGCCATTGGC
CCCTTGTGAGGCCAGAGACTGTGTGAACTGTCGAGCAACGGCTACTCCACTGTGGCGGAGGGACAGGACAGGTCACTACCTGTGCAATGCCTGT
GGCTTGTATCACAAGATGAATGGTCAGAACCGGCCTCTCATCCGGCCCAAGAAGCGAATGATTGTCAGCAAACGGGCAGGCACCCAATGCACTA
ACTGTCAAACGACCACTACAACACTCTGGCGGAGGAATGCCAGCGGAGATCCGGTATGCAATGCCTGCGGCCTCTATTTCAAGCTCCATCAGGT
GAACCGCCCACTGACCATGAGGAAAGATGGAATCCAGACGAGGAACCGCAAGGCATCTGGCAAAGGGAAAAAGAAGCGGGGGTCGAATCTGGCT
GGAGCGGGAGCGGCTGAAGGACCAGCTGGTGGCTTCATGGTGGTAGCTGGTAGCAGCAGTAGTGGGAATTGTGGGGAGGTGGCCTCAGGCTTGG
CACTGGGCACTGCAGGTACTGCCCACCTCTATCAGGGCCTAGGACCTGTGGTACTGTCAGGGCCTGTCAGCCATCTTATGCCTTTTCCTGGACC
TCTGCTGGGATCGCCTACAACCTCCTTCCCCACCGGTCCTGCACCTACCACCAGCAGCACCAGCGTGATCGCCCCACTCAGTTCTTGAAGGTAC
ACAGAATAGCCTTGACCTTGTGGCAGAGGAGACCCCCCCTTTTTTTGGAGACAGGATCTTCTGTAGCTATGTAGCTATGAACTATGTAGATAAG
GGTGACCCCACATTTCCCATTCTCCTGCCTCTACCTCCCGAGTACTGAGATTCAGGCATGTATTGCTATGCCTGACATTTTCCTTCCTTTCAGT
TTTGTTTTGTGGGGTTTGTTGTTGTTGTTTTGAGACAGGGTTTCACTATGCAGCCTAGGCTGGCTTTGAATTCCTTATGTAGTCCAGACTAAGC
TCCAGCTTGAGATCCTTGTGCCTCAGTTCCTGAGTGCTGGGATTACAGGCATGTGCTACCGTGCCCGTTTCTCCCTCCTCTTAGAGCCACTGGC
CTTTACAGCCCAACCATCTCCTTGCCCTGCACCCTTAAAGATTTTGTAAAATAAAACCACCTGGAGACATGCGCTGGTATTTGGTTAAATAAAT
GTCAGTGTCAAGTGGAGCATG
MOUSE SEQUENCE—CODING
ATGGATTTTCCTGGTCTAGGGGCCCTGGGGACCTCAGAACCCTTGCCCCAGTTTGTGGATTCTGCCCTGGTGTCCTCACCATCAGATTCCACAG
GTTTCTTTTCCTCTGGGCCAGAGGGTTTGGATGCAGCATCTTCTTCCACTTCCCCAAATGCAGCCACGGCCGCAGCATCAGCACTGGCCTACTA
CAGAGAAGCTGAGGCCTACAGACACTCCCCAGTCTTTCAGGTGTATCCACTGCTCAACAGTATGGAGGGAATTCCTGGGGGCTCACCTTATGCT
AGCTGGGCCTATGGCAAGACGGCACTCTACCCTGCCTCAACTGTGTGCCCCAGCCATGAGGATGCCCCTTCCCAGGCCCTGGAAGACCAGGAAG
GGAAGAGCAACAACACGTTCTTGGACACCTTGAAGACGGAGCGGCTGAGTCCAGACCTCCTGACGCTGGGGACTGCACTGCCGGCATCGCTCCC
TGTCACCGGCAGTGCTTACGGGGGAGCTGACTTTCCCAGTCCTTTCTTCTCTCCCACTGGGAGCCCTCTCAGCTCAGCAGCCTATTCTTCCCCC
AAGTTTCATGGAAGCCTGCCATTGGCCCCTTGTGAGGCCAGAGAGTGTGTGAACTGTGGAGCAACGGCTACTCCACTGTGCCGGAGGGACAGGA
CAGGTCACTACCTGTGCAATGCCTGTGGCTTGTATCACAAGATGAATGGTCAGAACCGGCCTCTCATCCGGCCCAAGAAGCGAATGATTGTCAG
CAAACGCCCAGGCACCCAATGCACTAACTGTCAAACGACCACTACAACACTCTGGCGGAGGAATGCCACCCGAGATCCGGTATGCAATGCCTGC
GGCCTCTATTTCAAGCTCCATCAGGTGAACCGCCCACTGACCATGACGAAACATGGAATCCAGACGAGGAACCGCAAGGCATCWGCCAAACGGA
AAAAGAAGCGGGGGTCGAATCTGGCTGGAGCGGGAGCGGCTGAAGGACCAGCTGGTGGCTTCATGGTGGTAGCTGGTAGCAGCAGTAGTGGGAA
TTGTGGGGAGGTGGCCTCAGGCTTGGCACTGGGCACTGCAGCTACTGCCCACCTCTATCAGGGCCTAGGACCTGTGGTACTGTCAGGGCCTGTC
AGCCATCTTATGCCTTTTCCTGGACCTCTGCTGGGATCGCCTACAACCTCCTTCCCCACCGGTCCTGCACCTACCACCAGCAGCACCAGCGTGA
TCGCCCCACTCAGTTCTTCA
HUMAN SEQUENCE—GENOMIC
AGCAGCTTTCCATCTCTGCTGTGACTGCAGGCCGTATTTGTCACATTCTTCTCTGCTTATTTTATGTTTTACAGCAAGATTCTCGGCAGTCATT
GCCATGGGGAGCTGGACATGCTGATCTGTTAATCCTATCCATAAAGAATCTTCCAGCTTGATATCTGATCCAAGCTTGGTTCCAAAACACACAT
TTCTGACACAGTAGGGAGCTTGGCTCATGCTTTTGGTTCCTCCGCATAAAACAACTTCAGCTTCTTTAACACAAATTTCCTAACACCTATTCAC
AGTGGACTGAAAACCAGAACCACAGAGCCTATTAATCGTGAGAGCTGGGGTCTCCTTTCGGATTCCCACACGCAAACCAACATGCCTTGCCAAA
AAATATGGAACGCTTCACGAATTTGCATGTCATCCTTGCGCAGGGGCCATGCTATATCTTCTCTGTATCGTTCCAGTTTTAGTATATGTGCTCC
CGAAGCAAGCATGGAGATGATTTTTTTAAAATGGTTAGATTCTGGAGATATTTTGAACATAGGGACTGGATGAGTTCAACAAAAAAGAGGATGT
CAGCTAGAGAAGAGGCCTGAGGACATTTTGGGTTCTAAAACAAGAGAAGGGACAGACAGATGACTCAGGCAAGTGGGAACGGAAGTGTGTGGAT
TCCTGAGTGAAGGAGTGATGGATGGAGGAGCATGCGTGAGAGTCTCCTGAAGTAAAGGAGCCCCTGGTCTTCTGGACTCTGGTCGCTAAATAGT
CTTCAGACGGTAGTTCAGCCAGACCCTGTGACTGAACCGCCAAGGAGCCCCAAGAGTGCTGCCAAGAGAGCGAGTGTGAGGATATGAGTTTTAG
CAAGTAAAAGGCTTTCCTAAAACCTGTAGCATTTTCTGGCAGTGGAAAAAAAGAGGAAAGGGGTTTTCTGCCTCATTCTAAAAGACAATTTGCC
ATGCACCCAGACATTATATGCCTGAAGATGGATGATTTATGGATGAAAGGAAAGAGAAAATGAAGGAATAGAAGAAGGAAGGAAGGAACAGATG
GGTGGATGGGTGAGTGGGTAGGTAGATGAATGAATGGGTAGATGCTCTGATATTTCCTCCCTCCTCCCCCGAGAAACTCCTTCACAGGTGTGCA
CAGAGAGAGGAATATGAGTTTATGAATTGCAGATTTGCTATAATAGGGAAAGAAACTAGAAACAATGCAAATGTTCATCAACAGATTAGTGGTT
AAACAAAATGTGGTATATCCATTCCATGGAATATTACTTTCAATAAAAGGGAACGAACTTATGAGGCCAGCATTACTCTGATGAGAAAACTAGA
CAAAGATATCACAAGAAAAACTACAGACCAATATCTAAGTATACATGCAAAAAPCCTCAAAAAAATACAAGTAAGTAGAATCCAGCAACATATA
AAAGGATTATACACCACGGCCAAGTGGGAWTTATCCCAAGAATGCAAGGTTTGGTCAACTAATGAAAATCAATCAATATAATGCATCATATTAA
CATAATAAAGAACAAACACCACATGATCTTTTCAGGAGATGCAGAAAAAACATTTGACAAAATTCAACATTCCTTCATGATAAAAACACTCAAC
AAACTAGGAATAGAAGAGAACTTCCTCAACCTGATAAAGGACATCCACAAAAAACCCACAGCTAAGATCATACTTAATGGTGAAAGTTGGAAAC
GAACAACACAAGGATGCCCCTTCTTACCATGTCTTTTCAACACTGTATTGGATGCTCTACCTGGGGCATTAGGCAACAAAAAGAAATAAAACCT
ATCCATATTGGAAAGGAAGAACTAAAACTATCCCTATATGCAGATGACATGATCTTATACATAGAAAATGTTAAGGAATCCACCACGAAACCAT
TAGAGCCCCATTAGTGCATAACCATTAGAGCAAGGTTGCAGGATATAAGATTTTGTATCATATATGCTAGCAATTAACAATCCAAAAGTGAAAT
TAAGAAAACTATTCCATTTACAATAGCTTCAAAATGAATAAAATACTTAGTAATAAAATGAATGAAAGAAGGGTAAGACTTATACACTGAAAAC
CATAAAATATTATTGAAAGAACTTAAAGAAGACCTAAATAAATGGAAAGACATCACATGTTCATGGATTGGAAGACTTTATAGTGTTAAGATGG
CAATACTCCCCAAATTGATCTACAGACTTGATGCAATCCCTATCAGAATCCCAGCTTGCTTCTTTGCAGAACTGACAAGCTGATTCACATGGGA
GTGCTAGGGGCCCAAAATACAACAATCTTGAAAAAGAAAAACAAAGTTAGAGGACTCACACCTTCCAGTTTAAAAACTTACTATACAGCTACAG
TAATCAAGACCATGTGCTACTGGCATAAAAACATACATATAAATTAACGAAATAGAATTGAAAGTCCAGAAATAAATTCATACATTATAGGCAA
CTGATTTTTTTGAGACACGGACTTGCTCTGTTGCCCAGTCACAAACATAGCTCACTGCAGCCTCAAACTCCTGGGCTAAGCGATACTCCCACAT
CAGCCTCCCAAGTAGCTGGGACTACAGGCATGCACCACTGTGCTCAGCTAATTTTTAAAAAACTTTTTGTAGAGACAGGGTCTCAATGTGTTGG
ACAGGCTGAACTCCTGGGTTCAAGTGATCCTCCCACGTTGGCCTCCCAAAATGCTGGGATTACAGGTGTGAGCCACTGTGTCCAGCTAGCAATT
GATTTTTGACAAGTGTACCAAGACCATTCAATGCGCAAAGAATGCTCCTTTCAACAAATGGTGCTGAGACAGCTCAGTATCCACATGCAAAACA
ATAAAGTGGGACCCCTTCCTCATACCATATACAAAAAACAACTCAAAACAGATCAAAGACCTAAATGTAAGAGCTAAAACGATAAAACTTTTAG
AACGGGGGTCCCCAACCTCCAGGCCACAAACCCATCTGTGGTCTCTTAGAAACTGGGCCACACAGCAGGAGGTGACCTGCGGCTTGCTGCCATT
CATGAAGCTGAGCCCGCCTCNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNAACCTACAGAAGCACAATATTTGAAAATCACATATCTAAAAAGGAGTCTAGTATCCAG
CAATACATAAAGAACTCTTACTACTCAACAATAAAAGGACAAATAACCCAATTCAATGGGCAAAGGATTTAAATAAAAACACTTCTCCAAAGAA
GATAAATGGGCTGGGCACGGTGGCTCATGCCTGTAATCCCAGCATTTTGGGAGACTGAGGCGGATGGATCACTTGTGGTCAGGAGTTCGAGACT
GGCCTGGCCAACATAGTGAAACCCCATCTCTCCTAATAATACAAAAATTAGCCAGGCATGGTGGCGGGTGCCTGTAATCCCAGCTACTCAGGAG
ACTGAGGCAGGATAATCACTTGAACCCAGCAGGTGGAGGCTGCAGTGAGCCAAGATCGTGCCACTGCACTCCAGCCTGGGTGACAGAGCAAGAC
TACATCTCAAAAAAAAAAAAAAAAAAAAAAAGAAGATAGATGACCAACAAGTTTATGAAAATATGCTCAACATCAGTGGTCACAGGGAAATGCA
AATCAAAACCATAACAAGAWACCACTTCACACCCACACCCAGTAGGATGGCTAGAATCAAAAAGAGGGACAATAGAAAGTGTTGGAGGCCAGGC
ATGGTAGCCTGTAATTCCAGCACTTTGCAAGGCCAAGGCAGGCAGATTGCTTGAGCCCAGTAGTTTGAGACTAGCGTAGGCAACGTGGCGAAAA
TCCCATCTCTACGAAAAATTAACAAAAATTTAGCTGGCTGTGATGGCATGCACCTGTAGTCCTGCCTACTCAGGAGGTTGAGGTGGGAGGATCA
CTTGAACCCAGGAGCTTGAGGCTGCAGTGAGCAGAGATCATGTCACTGTACTCCAGCCTGGGGAACAGAGTGAGACAACCTCCCACAAAAGAAA
AAAGAAAAAAAAGAAAAAAGAAAGAAAGAAAGAAAGAGAGAGAGAGAGAGAGAGAAAGAAGAAGAAGAGAAAAAAAAGAAAGAAAGAAAGAAAG
AAAGAAAGAAGAAAGAAAAGAAAAGAAAAGAAAAGAAAGAAAGAAAGAAAGAAAGAAAGGAGTGTTGGAGAGAATGTGGTTAAATTTAAATTTG
AATCCCCATATGGCAGCTGTGGAGAACACTTGCAGTTCCTCAGAAAGTTAAACATAGAATTACCATATCACCCCGCAATTCCATTCCTAGTTAT
ATACCCAAGAAAACGAAAAACATATGTCCACATAAAAACTTTCACATGAATGTTCATAGCAGCATTATTTGTGATAGCCAAGAGTCCATCAATG
GGTGAATGGATAAGCATAATGTGGCATTTCTTTTTCCTTTTATCTTTTGAGACAGGGTCTCACTCTGTTGCCCAGGCTGGAGTGCAGTGGTGCA
ATCATGGCTCACTGCAGCCTCAACTTGCTGATCCTCCCACCTGAGCCCCCCAAGTAGCTGGGACCACAGGCATGCACCACCACACCCGGCTAAT
TTTTCTTTTTTGTACACACGTGTTCTTGCTACGCTGCCCAGGCTGGTCTCGAACTCCTGGGCTCAAGCGATCCACCCACCTCAACCTCCCAAAG
TGCTGAGATTACAGGTGTGAGCCACCGCATCCTAGCTATAATGTAGTAGGTATTTTCATACAAAGGAATATTATTCAGTCATGAAAATGAATGA
AGTACTGATACATGCTACAATATGAATGAACCTTATTACTTATGTGAAGTAAAAGAAGGCAGTTACATATAAGGCCACATATTATATGACTGCA
TTTATATGAAATGTCCAAAACAGACAAATTCATAGAGACAAAGACATTTGTGATGGCCCGGCGATGGGAGGAGAGGGAAATGGAATGGGAGGGT
GTATGTTAATAGATATAGGGTTCCTTTTCGGGGTGATGAAAATGTTCTGGAATTAGGTAGTGGATAGTTACACATCCTCGTGATATACTAATCG
CTTTGCTATACATTTTAAGAGTGAATTTTATGGTTAAGAGCAAATTTTATGGTATGTGAATTATGTCTCAATAACAGAAAAAAAAGAAAAAAGA
ATGAACTATTGATACACACGACAATGTGGAAGAATGTCACAGACATTATGCTGAGCAAAAGCAGCACATACTGTATGATCTGCTTCCATTTCCA
TGAAGTGCAAAAACAGGCCAAAACCCCAGGTATCATATGGTGTTTATGGGTACCAACATATGGAGTGATGGAGTTATAAAAATACTCATGAGGA
AGATAAACACCAAGCTCAAGGCAGTAGAAGGAGAGAGGGAAATGGAATCAGTACAAGAGCATCTGTAATATCGCATATCCTTTAAAAACAATCT
GAAGTCAATAAGGCAAAATATTAATGTTTGACAAAAGTAGATGCTTGTTACAGCACCCTTTATACTTTCCATTTTTTAAAACAGTCAACCAACA
GTTTGTTTCTGAGGGCAAACGCTTGACTCCTTAACCAGGCTTTGGTTGACATTTGCCTTAGTGCTTTTGACACCACAGTCTGTCAGGCTCTGTC
CCCACCCTGATGGGCTCCACAGTACAGAAGAGGCACAACAGTACAAATGCCATGGGGCCTAACGACCACCAGGGGCTCTAGAACCCCAGAAGAT
GCCAGGAGGGAGTGAGCCAGTCAGGGAAGGCTTCCGAGAAGACAGCACATTGAACAAGAGTCTCAAACTTAGGCCTGACGGAGAAGACGCGCGG
CCAGGACACCCCACCCCCGCCCTCGTCTCCCCCAAAGCCTGATCTGGCCCCACTCATTCCCTTATCTGCCCACTCCCAGCTGCCTCCTTGCTGG
CTCAACTGTCGCCGCAGACTTCTGAGCCTGCGCCCCCTCCACGGGGATGGGGGAGGGAATGGGGTGAGGCCTCGCCTCACAGCCTCGGGCTTTC
CAGCTCTTCCTGGAGGCAGGGCTCTGGGGCGCCCTACTCCTCACCCTTGGCTTCTCTTCCTGAGCGCTCTGTGCTCTCCAGAAATCAAGAAATG
G~GTCAGTCCACCCCCCAAACCCTTGTCTTAGCTCTTAGACATGCCTCGAGCCTGCCATTCCCTGTGAGCACAGATTTCCCTATGTTGCGACCG
CTGCTTCTAATAATAATAATGATGATGATAATTCCCATTTACAGATCACACCATTTATGGTGTGCCAGCAGGCCCTGTGCTGAGTGGTTCCTAC
CCACGTGGGGGGCTAGCACTTTACCCGTTTTCCAGATGAAGAAACTAGAGGCTCAGAGGGCGTCTGGCCCAGGAATCACACAGCAAMCACACAG
CAAATCAGAGTTGAGACAAGAACCCAGGGCCCTGGAGGAACATCTTCATTTCCACACACCTATGTAGAACCAGTCTGCAGGTGAGGGCAATTAT
ACACACTTGCACCACAAGGAGCTGGTGAGAATGCGATAGAATTATGGGTGAAGAACCCTTAGCACAGTGCCTGGGGTCACTCAGCAAATAATGC
CGCTGCTGTTATTATTAGTCTATTAATGCTATTATTAATATCAGAATTAAACAAGCCTCGGCAACATAGCGAGACTTCATCTCTATTGAAAAAA
AATTTTTTGCTGGGTGTGGTGGCATGCACCTGAAGTCCCAGCTCCTCAGGAGGTTGACGTCGGAGGATCACTTGACCCCAGGAGGTGGACGCTG
CAGTGAGCCAAGATTGTCACTGCACTCCAGCCTGAGTGACATAGTGAGACACTGTCCCCAAAAAATATTTAGTACGTATCATTATCACTATTGT
TGTTAGAATTAGAATAGTTTTAGAATTAGTGTTAGTATTAATAATAGTATTAGTGTTAGATTAAAATTAGTATTAGCATTAGATGAGTATTACG
ATGAAAATTAGTATGTCCTCATAGAAATAGAGAGTAGAATAGTGGTTACCAGCAGATGGGAGGGTAGGAGGCAGGGAGGGATGGGGAGAGGCTG
TCAATGGGTACAAAGCTACAGTTAGATAGGAGGCATAAGTTCTGGTGTTCTAATGCATAGTACAATGACTAGAGTCAACAATAAATGTATATTT
CAAAATAGCTCAAAGACGCCTTCTCGAATGTTCTCATCACAAAGAAAAGATAAATGTTTAAGGTGATGAATATGCTAATTACCTTGATTTGATC
ACTACACAATQTATACATGTAACAAAACATCATGTTTTACCCCATATATATGTACAACTATTATGTGTCAATCATAAATTTAAAGAAATAAAAT
TAGTTTTAGTGTCAGTATCAATATTACAATATTGGTATTACTCTTAGTGTCAGCATATTATTAGCATTAGTACTATAAGTAGTAGTAGGGTTAG
AAAGGGCTATGTACTCTCCCTTGTATGCAGTAGGTACTCAATAAATAAATAGGGGTACTTAATATTTATTGAGATTTCAGATTCAAGTAAGTTA
AGATCATGTAACATGGGTTAAAACTCATTTAGTCTCCCCAGTCACTGCATGGAGTGGCTTATTGGGGTATATTATTACCCCATTTTTACAGAAG
AGGAGCTAAATTCCAGAGCCGATGGTAACTCTCCCAACGTCACCTGATCCATTAGTGGTTAGCGCAGTCTATGAACCAGGATGAGCTGCCTCTA
GAGACCATCTCCTGAAACCACTGCCACTTAAGTCAGAGGTGTCTTTGTATTCGTTCTCCTTATTACTGATGAGGGTTCAGCATGGGGCGTGGCA
CACCAGCAGGAGCTCAATAAATAGGAATTCATGGGCTTGCTCTCTTTCCTCCTCAAATCTCCCATTGCATATGAGGACACTGAGGCTCAGAGGA
GTGAGATAAGAATGGTAACATCCCTCCAGGGTGGGCCAGTGGGCAACCTCACCCCACTCACCTGCGGCTCCACCCTPGCCCTCTGCCCTTTGTA
GGTCTCAGTCTCCCCATCTGTAGACTAGGTGGGCAGGACAGCTCCTTGTCCCACGTGTCCATGTGTGTCAGATGCTTTCATATAGAAAAAGTGC
TCAGGTTAGGTACAAGTCTATGTGAGGTCTATGAGACACTGTGGTTGTTGTCATTAATATTTGGAGGAGGTGGGGCAGGGCAAGAGTGTGGGGA
CTCGGGTGCGGAGGCCTAGCCACATTCTGGTTGTCCCCTGCTGAGGGCTGACGGCAGACCCACAGGCTACATCAATCCACATCCTCCCATCCTA
CCTGCATGGGCACCACATGCCCAGGGTGTGATCCAGTCTGCCTGGACCTTCTTCTGAGTCTGTCTCTCAATATCCCTCTGTCTCTGTCTCCCTT
CTCAGTTCTCCACTTCTGTCTTTGGCCTCTTTCCCTTTCAGGTTTTATCTGTCACTTCTGTCCATCTCTGTCTCTCATTCTCTCTTTGCCTCTC
TCTATATGTCTTTAATGGTCTCTGGTTCTCCCCGGGTGTCTCGCCCTCTTTGTCTCTCCTTTTCTTTTGTCCCCTCTCTCTGCCTCTTTCCACG
TCTGTCTCCTTCTCTAT&CATCTCTGTCACTCTCTTTCTCCCCATTTCCATCTCTCTCCGCCTATCTCTGTTTTTGTTTGTCTCTCTGCCTCTG
TCTCTCCCACTCCACCCCTTTCCTTTCCTACCCTATCCCACTCCTCGAGGAATCATCCCTGGCTCCCACCTCAGTTTCCCGCCTCCAAGGCAGC
ATGGCGGGCAAGAAGTTGAGGCCACTGTCCCTGGGTGTTCCTACCCCCACACCCTCACCCCAAGACAGCCTGTTACTGCGGCGCCAACAGCCAC
GGTCGCCTACATCTGATAAGACTTATCTGCTCCCCCACGGCAGGCCGGAGCTGGCGTAAGCCCCATGGGGCCACTAAGTGACTGTGCCCCTCCC
TCCCGCCAGCACTCGCCTGGCCTGCACGCTTAGCCTGGGTCATCAAGGTATCCCACAGGCTCTAGTTCAAATCCAGCAGAACCTCTCTGAGCCT
CACTCTTCTCACCTGCAAAATGGATACAGCCACATCCCTTCTCTCCCTGCAGCCAGGAAGACGCACATACACAGGAGTCTAGCCCACACCGGCC
CCGCACAAATTAAGGGCTTTACTCTCTGAAAACCCCAGTCAACTCATCAAACCATATCTGCTATTTTCATTTATCTTGGTTTCACCCTATTTTG
CTTGTCTGGACACTACAGTCCACGGGAGCCTAGGTCGAGCGAGGTCCAAGAATCCCCAGGGTGGGCAGGGAGGGTGGAAGAGGGCCTCCAGTGC
CCAAGAGGTGCCCCACAAGCATGGGACCCCCCCCCTCCCCTCGACTGCCCCACCCACTGGGGCACCAGCCACTCCCTGGGGAGGAGGGAGCACG
CAGAAGGQAGGQAGGGAGGGAGGGAGGAAGGGAGCCTCAAAGGCCAACGCCAGCCACGACACCCCCTGGCATCACACTGAGCTTGCCACATCCC
CAAGGCGGCCGACCCTCCGAAAACCACCAGCCCAGGTCAGTCTCAGCCCCCAGAGAGCCCCCACCAAGGCAACCCTGGGCCTGCTGCCCCTACC
CTTATGCTTGCTCCAGGCTCCCCAACCCACAGCTGGTCCCCAAAGTGTCAGCCAGCCCTGAATCCCTTTACAGCTCCTCCAACCCACCTGTCTT
TCCAACTCCCATTTCTACCCCAAATCTACCCCCTAAACCCCACCTCTTTCTGCCCTCTCATCCACTCCGTCCAAGATATCTATATCCTCTGGAT
CCCTTCACTTTGCTCGCTATACCCCTAGGTCTGTCTCCCCTAAACACTAACTCTGTCTCCCAAAACATGACTTTATCTCCCCAAATCCTTTCCC
TGTCCCATATCTCATTTCCACTCACCCAAACTTGTCTCTGTCTCCCTGACTTCTCCTCCAAACCTCACTTTTCTGCTCCCAAACCCCTTATCTG
CTCTACAACCCCTTGTCTTTGCCCCACTCTCTCTGAGCCCCCAAACCTGTCTCTGATCTGCCAAACCCTAGCTCCATCCTCTTACCACCATCTC
TGCCTTTCAAACCCACTCTTTGTTCCCAATGACTTCTCTCTGATCTCCGCAAACCATTTATCTCTCCCAACTCCCTGCCTGCTCAGTAAAATAT
CTTTGTCACTTCAAACACCAAGTCAACTCCCCAAAATCTGTTCTGCCGCATGAACCCTCATGTCTGCCTTCCCCAAACCGTATCTCAGCCCTAA
AACACATCTCATTGTCTCCCTGAATCTCTCCAGCTCCAAGCCTTTCTCTACTCCAATTCTATCTTGGCTTCTCAAACTCCCCTTTACTAGCCAC
ATTCTGTCTGCCCAAACAGATCTTTGACTCCTAAACTCCATCPCTCCTCCAGAAAAACCCTTGAACTCCCCAGACTTTTCTATGACCCTCAGAA
TCTGACTCCCACACTACTTCTCAACTTTATATCTGACTCTCAAACTTCGCCCCGATCCCCCAACCTCGTCTGTTTTCACAACCTATTCCTGAGC
CCAAAATGTGTCTCTGAACTTCCAAGCTCGTCTCTGCTCCACAACCCCGCCCATATCTGCCTCCCCACAAACTCCATGGCTACACCCCAAACCT
CTCTGACATTACCCCATATCTGACCTTCAAATTCCACCTCTATCTCCCCCTAACCCCCATATCTGCCCCCTAGACCTTATATCTGCTCCCTTCA
GCCCGGTGAGACCCTCCCTTGAGGGCTGGGATTGGAACGTTACCTGGGGTGAGGGGCTGGGGCCGAGGGAAAGTGGACCGTGCCAGCTGCTGCC
TGCTGCCGCCGTTGGGGCAACACTTACTCTAGTGGGGCAGCTGATAAGGAGCTTTCATATCCCCCAGCCTCGAGATAAACTTTATCTCTGTCCG
GAGAGTGATAACTGGGGGTGAGGAGGCTGGGCCCCTGCCATGGGAGGGGTGGGCAGCCCTGGACTCACCAACGTGTGAGGTGCAGGGGPTCGGG
GCAGTGGGGCTGGAGCGAGATCATGGTGGGTAAGGTGGAGTTGAGGGGATGAGGGAATACTGGTGAAAGAGTGAGCAGAAGACACACAATCAGA
CAGGGAAGAGAGGGAGAAACAGGGAGAGATGGGAAGAGAGAGACAGACACACAGAGACAGAGCAAGAGACAAAAAAGAGACATGAAGAGAAAGG
AAAACAGAGAGAGAGAAACTCAACGAGTAAGAGACAGAGACAGACAGATGGCCGGAGAAAAAAGAACGAAAGACCCCGGAGCAGAAAAGGAGAT
AGAAATGCAGAGATACACGCAGCTAGCACTGTGAAGAGGCAGAGACTAACACACAATTAGAGACAGAGAGGAAAGAGATGGAGACCAAGATGGG
GAAGCAAAACACTCTCTTCAGACAGCAACAGCAGGCACCCTGGCGCTGACAAGGTGTTGACGGTGGGCACACTTGTGGCCCATCTCTTTCCTAG
CATAACTCCCAAGCCTGGGCCTGCCCCTTAGCCTGTCTATACCATGGGTCATGTAAGCAGGAGTGAGGGGCAGTTTGAAGACAGCCTTCTAGTC
TAGCCCTGTCACTCCACCCTACAGACACAGAAACAGAGCAGGCCAGCACAATAGGGCCAGGGCCAGCATTCCAGGCTCCTTATCCTGAGCTGCA
TGTGGCTGGGTACGGTCAGGGGTATGGGACTAGTAAGAGGCACGTGGACATAAGGTCAGAGGCCCCAAAGAGAGGGAAACAGAGAGAACAGACA
AACAAGCAGGAAGAGTGGGAGAGATGCAGAGATGTTCAGAGAGATAGGAGAGGAGAGGGAGAAAGCAAGAGTAACAGAAAGAGAGAGCCAGAGA
CAGAAACATGGGAAGACAGAAAAGAAGACAGAAAATCATCAAACATTACAAAAGCACAGAAACAGGTCGGGCGCAGTCGCTCACGCCTATAACC
TCAGCACTCTGGGAGGCCGAGGTGGGCCGATCACTTGAGGTCAGGAGTTCAAGACCAGCCTCCCCAACATGGCAAAACCTCGTCTTTACTAAAA
ATACAAAAATTAGCTGGGTGTCCTGGCATGTGCCTGTAATCCCAGCTACTAGCGAGGCTGAGCCACGAGAATCACTTGAACCTGGGAGGCTGGG
GTTGCAGTGAGCTGAGATCGCACCACTGCACACCAGCCTGGGTGACAGAGTGAGACTCCGTCTCGAAAAAAAAAAAAAAAAAAAAAGAAAAAGC
ACAGAAGGAGAAGACAAGCTATATCATAGAAACACAGAAAGCTGGGGAAGCTACAGAGTCAGACTTGAGAGCGAACAGAGAGTCAGGGGAAAGA
GCCCCAGGGGATAGACAAGTGCAGAGCTAGATGACAGACACACACAAGGCAAAACAGGCAAAGGGCCATCCCACAGGTCTCAGCTCAGCCCCAG
CCCATCACCACCCCCAATACAGCAGATCGCGAAACTGACGCCTAGGAACTGAAAGAGCCTGAAAGCAGAACTCTGGTGGGCCTCTCCAGGGGAG
AGGGTACCGGTAGAGGTACCACCCACCTGCCACCCTCTTCAGAGGAAGTCAGTGGCCTTGGGCTGATTTCAAAAGTTGGGCGGGGAAGGCAGAG
ATAAGCAGTGGGGGGTACTGACCCCCCCCCAACCAAGAAGTAGGGAGGAACTAAGGGGGCCTTCTGTCTGTAGACGCAACAGAGGTGGAGGGAG
GAGGGAGTCAAGCCCGGAAACCATGGGGTTTCTGAGAAAGTTAGAGCGCAAGATACAACAGATAGGGATCAAGTTGGGGAGCAGAGGATGGTGA
ACCCCAAAGTCCTGGATGAAGTGACCAAGAGGCAGGCACTCTCGTCCTGCATGCCATCCCACCCTCACCCAGACTCTTCTAGAGAGGGAAAGGA
AGAAAGGACGGGGGGACGGGGAGAATAAGAGGAAGTGGAGCAGCGGAGGAGAGGAGATGGGGAAAGGGAGAAAAGGATGAACAGAATAGGGAGG
AGAAGGATGAAGAAATTGGAGGGGAGGACAAATGAGTAGTAGCAGAAACGAGTGCAGAGTAGATGTAAACAGGGAAAAAGAAGCCGGAAGATGG
TGGCTCACACCTGTAATCCCAGTACTTTCGGAGGCTGAGGTGGGAGGATCACTTGAGGCCAGGAGTTCAAGACCAGCCTGGGCAACAGAGTCAT
ACCCCATTTCTACAAAAAAAAAACAAAAACTAGCCAGACCTGGTGGCACACACCTGTAGTCCCAGCTACTCACGAGCCTCAGCCACGACCATCA
CTGCCGGGA~GCAGTGGTTGCAGTGAGCCGAGATCGTGCCACTGCACTCCAGCCTCGCAACACAGCAAGACCCTGCCTCAAAAAAAGTGTGTGT
GGATGCGGCAGAG~GAGATAAGGTGTGTGAGAAAGGTAGAAAAGGAGAATGAAGACAAAAAGAGAAGGAGGACAAGGAAGAGGGGAAACAGGAA
AGGAGAAGGAGGAGGTCAAGGAGGAAAAGAAGAAGGTGGAGGAGAAAAACGAAGCAGGAGAGAATGAGAAAACAGTCGAGAAAAATAGAAATGG
GGAAGAGGGGGGCAAAGAAGAGATGGGAAGAAGAGAGACGCGACAAAGACAGGAGGAAAAGGGAAGGGGGGAAGAAGAGAGAGAATGGAAGGAA
GAAGGGAGAGGTAGAAGCAGATGAGAGTGGAGGAAGAGCGAGAGGAGGGAGGAGAGGAAAGACAAACGGCAAGAGGGGCAGAACGAGGAGCAAG
ATGAAAGGAGGGGCACACAGGAGTGGAACGGGGAGATGCAGGAGGGAAAAGAGAGGAGAGGGAGGAAGAGAAGGAGGGGAAGGAGAGGGGAGAC
TAAGGTGAGGAGGAGGGGGGAATGGGGAGGTGGGAAGGAGAAATATGGAGACTGAGGTGATGGAGTGGGAGGAGGGGGAAAGGAGGGAAGAGGA
GCAGGTGAAAGGAGGTGGGGGGGAAGGATTTCTGTGTCTGAGGACCCCTTCTGTCCTCGCAGGTTAATCCCCAGAGGCTCCATGGAGTTCCCTG
GCCTCCGGTCCCTGGGGACCTCAGAGCCCCTCCCCCAGTTTGTGGATCCTGCTCTGGTGTCCTCCACACCAGAATCAGGGGTTTTCTTCCCCTC
TGGGCCTGAGGGCTTGCATGCAGCAGCTTCCTCCACTGCCCCGAGCACAGCCACCGCTGCAGCTGCGGCACTGGCCTACTACAGGGACGCTGAG
GCCTACAGACACTCCCCACGTAACTCCATTCAGTGGCTGTCTTAGCATTGGCTGAGTGCTGTTGGGGTTGCCATGGAGATCCTTGGCTAGGTCA
GAATACCACTGTGAGGATATCTCAGAAATGGCTGGAAGCTTCTCAAATGGATGTGCCGACCACTTTCCCTAGTTAAGTGCAGACCTGGGAATTC
CAATGCTCCTCAACCTGCCATATTGGGGCGGCCACACTGAGAGGCAATACTGGAAGTATGTGGTGGTTGCCCTAGTTGTCGAGTGATCCGTGGA
GCTCCAAATCCCAACAGTCATCCTCAAAAACCCACTTGGAAATGGTCAGAGCTTATTGCACAGGCCACACTGACCAGTGGGGGTCAGGATCCAG
GAAGCATCCAATGGCCAGCACCTGTTCTGGTAGCCTCTGGAAAAGCTGGGAACTTGGCCACCATGTTGGGGGTGCTGGGAACCACTGCACCCTG
ACGTGCGCTGACCCTAGACTGATTTTGCCTCTTCTTTCCTCCATCCCTACCTGCCCCCAACAGTCTTTCAGGTGTACCCATTGCTCAACTGTAT
GGAaCGGATCCCACGGGGCTCACCATATGCCGGCTGGGCCTACGGCAAGACGGGGCTCTACCCTGCCTCAACTGTGTGTCCCACCCGCGAGGAC
TCTCCTCCCCAGGCCGTGGAAGATCTGGATGGAAAAGGCAGCACCAGCTTCCTGCAGACTTTGAAGACAGAGCGGCTGAQCCCAGACCTCCTGA
CCCTGGGACCTGCACTGCCTTCATCACTCCCTGTCCCCAATAGTGCTTATGGGGGCCCTGACTTTTCCAGTACCTTCTTTTCTCCCACCGGGAG
CCCCCTCAATTCAGCAGCCTATTCCTCTCCCAAGCTTCGTGGAACTCTCCCCCTGCCTCCCTGTGGTGAGAAATTCAAAAAAGGACAGGGAAGT
TGAGGTGGGAGGGGTGGCCCAAAGTAAAGCTGAGCTGAGCCTAGCTCCCTCTTCTCCTCTCCACCCCACCAGAGGCCAGGGAGTGTGTGAACTG
CGGAGC~CAGCCACTCCACTGTGGCGGAGGGAACAGGACAGGCCACTACCTATGCAACGCCTGCGGCCTCTATCACAAGATGAATGGGCAGAAC
AGGCCCCTCATCCGGCCCAAGAAGCGCCTGGTAAGACCACAGACCTGCTTCACCTTATACACAGGAACCCCTGTCCTCACCCTGTACAAGAAGC
CACATCTTCCCATTATATAGGAACGCTGTTCTCATGATTACAGGAAGCTACTGCAGGCCACCCTGTATGGGAACCCCTGTCCCAATATCACATG
GAAACCCTTGCCTTCATCCTACACAGGAAAATCTCCTCATCCCATATGGAAATCCTAGCCCTCACCCPGCATGAGAACTCCTGCCCTTTCCTTG
TACAGGAACCCAGATCTATAGAAGCTCCTATCCTCAACCTACCCTTATAAGCTTCAAACAGAAACCTTTGTCCTCACCCTGCACACCAGTGTTT
TCGTCCTTGGCTTCACACTGGAATCACCCCAGCTCCCTTCTCCCAGATGTCCTCATGGAATTGGACTGGGATGCTGCTTGCGCAPCAGGATATT
TTAGCTCCCCAAGTGATTCCGATACGCAGCCAAAGCTGAGAACCACCAATGTAGTCCCCCATAGAATACAGAACAAAAGAAACTTATACCAGAT
AGGAACCCCTGTACTGGCCCCATACACAATCCTCAGGCATCACCCTGTAAACAAAGCCCTGCCTTCAACCTGACCCTCACTTCTTGGGTCCTCC
TGACATCCCCTGTCAGCCCCTTACCCCCACTTCCACATCCCCAGGGCACTGATCTCACATCCTGTCGTCCCCTAGATTGTCAGTAAACAGGCAG
GTACTCAGTGCACCAACTGCCAGACGACCACCACGACACTGTGGCGGAGAAATGCCAGTGGGGATCCCGTGTGCAATGCCTGCGGCCTCTACTA
CAACCTACACCAGGTGACGCCCTGCCCCTTGGAGCCACCCCTCTGCTTTCCCTGTCTTCATGCCACACTGCCCCCCACTCTGTTCCTGTTCTAC
CTCTCTCTTCCCCCACAACCCTCTTTCCTCTTTCCCCTTTCCCTCCTTCCTCCCCCTTCCCCCATCTCTTCTACTTTCCCCCTTCCTCCATCTC
TTCTACTTTCCCCCTTCTCTGTTATTACCCTCCCTCCTTCCTTCTCCATTCCCTCCTCCTCCGCCCTCCTCCTTCCTCTCCTCTCCCCCACCTC
CAGGCATCAGCTAATGTCCAACCCCCTGCCCTAGACCTTGGGCAGCTCCTATCAGCCTTGGAGGCTTTCCTAAGCTCAGGGCCTCACAGCCTCA
GGATTCCTTGGACAATCTCAGCACCCAAAAATTATCTTACCCTGAAAGAAGTGGGGTAGAGAGGGTGTCCCTGGTTGACACAGAGAGGCAAAGG
TCTGGAGTTGGGGACACCCGCAGCCTCCTTTTTGGCAGGTGAACCGGCCACTGACCATGCGGAAGGATGGTATTCAGACTCGAAACCGCAAGGC
ATCTGGAAAAGGGAAAAAGAAACGGGGCTCCAGTCTGGGAGGCACAAGAGCAGCCGAAGGACCAGCTGGTGGCTTTATGGTGGTGGCTGGGGGC
AGCGGTAGCGGGAATTGTGGGGAGGTGGCTTCAGGCCTGACACTGGGCCCCCCAGGTACTGCCCATCTCTACCAAGGCCTGGGCCCTGTGGTGC
TGTCAGGGCCTGTTAGCCACCTCATGCCTTTCCCTGGACCCCTACTGGGCTCACCCACGGGCTCCTTCCCCACAGGCCCCATGCCCCCCACCAC
CAGCACTACTGTGGTGGCTCCGCTCAGCTCATGAGGGCACAGAGCATGGCCTCCAGAGGAGGGGTGGTGTCCTTCTCCTCTTGTAGCCAGAATT
CTCCACAACCCAAGTCTCTGGGCCCCAGGCACCCCCTGGCTTGAACCTTCAAAGCTTTTGTAAAATAAAACCACCAAAGTCCTGAAATTGTGGG
TGTQTCTATGTGTGGCTGAACAGCAGAGCTGAGTGCAGTGTGTAACAATGCCTGTGGCTCTCTGATQACAGTGCATGQCTGCAATCTGTGACCA
TGTGAGGCTGGCTGACGCGGCACATGTGACACTTTGTGACCAAGGGTATATGTTGTGTGATTGGACAATCTATGTGAAACTTCTTGTGAAGTGT
CCAGGTGGTTCCATATGTCATGGTAAGAGGCCACGCAGGGCTTGCTGACGGTGGGGGTGGTGCCGGGTGACTATAAGCACACTGGAAGTGTGTG
TGCTGGAGCAGGTGATAAaGAGGTQTQCGCQGCTGTAGTCTTGTGGCTGGGCATGACGCTGGGGGACCTTTGAGACGGCATCACGCTGGGGGAC
CTTTTAGACAGCATGACAGCATACGTGGTGGGCACAGAATCTGGTAAAGATTCTTGGAATGTTGAACAAGAAGTGTGTGTCAGGATGTGTTTGT
GCCTCTGGCGATGGCAATATTTCAGTGCAAGTCCCTGTGCATGGGCATGGGTGTGCGTGTGTGTGCATTACGTGTGGCATGTCCCATGTCACAG
TGTCTCTGAGTGGCTGACAACATGAGCAGCTCTGTGTACATTTGTTTGTGGAGGTCTCAACAGCAGAGTTTCAGAGGGTATAAGTTAGGGACTC
TCTTCCAAGGGACAAAAGACACATTCAGGAAAGGAACACGGAGAAGGGTGGAGCTGGAATGGTAGCTGGCAAGGTCAGAGGCCAGTGCTGGGGC
GCAGGGTTGTCCGTGCGTCTTCCTGTCGGACTGGTCTTATCTTGCTCAGCCCAGCTCAGCCTGCAGCCCTGTGGGAAATGGTAGAGAATGAGAG
GGGGAGATTAGGGGGACAAAGCAGCCCCTGGAGGTGGTTTCAACCTCAGTAGGTGGGGCGCTCCACACAGTGCCAACCCCTTCCCCAACATTCC
CACTCACCTGGGTGAACTGCTCCCCCCACAGCGGAGTCTGTTCCTCAATTTCACAAACACACAGGAGCATAATCTGAGTCATGCCTTAAGCATC
CTAAGGCAGACTGCTTAAGTGGCTCAATGGAGGCCACACAGCCAGTGGCAAGGGGCCCAGCGGGGCAGTCTGGCCTCGTCTCCATGTGCTCAGC
CACACCCCACAGCCAGCAGCACCCTTTCCTAAGGGCTCACTGTGGGTCAGGCGGCAGGCTGGGAGGCTCCAGTTGCTTTAGTTCATTAGTGCCT
GAAATAAATATTCTGAGGAAAATATACCCATACTCATTTCACTAATCGAAGACTCAGGCCCAGAACTGATGAGTAACTTACCCTGACCACCAAG
TTTAAGGAGTGGATTTTGGATGGGGTGACACACCCAGCCCAGCAAATAAGTGCTGCTCACACTTAACCGCTCTTTATGGCTTCTTCCCTAGAGC
CCCGGATATGGGGGTTGCAGGGACCCACAGGTCCTGACCATATTATAGGTAAGCAGCTGTCCTCCACAGAACACCAGCCCATCAGCTAGCGGGC
ATTCCAGCTCCACTAAGTAGCCTGCTGCAGAATCACAGGAGTCTATTATGCTCACTGTCGGCATTATCCACAACAGCCCTGATCCCATAAAAGA
CACGTGTCTGGCCTCCCCTCAGAAGCCCAGCCCTCACACCCTGCACGACGCCCCATCCTCTAAAAACCCCCTTCAGAACCTGTCCTCGACACAT
GCCTGGCCCCACCTTGTTCAGGAGCCCCGGCCCTCACCCCCATAAATATAGCTCCCCCAGCCTGGTACAGAACACTAGCTCTGAAGCTCTAACG
GAGCCCTTCATCTAATTATCGACAGGAACCCATCCTGATCCTGTACAGGAATGCCAC3CGCTTACCCTGAATAGCAGGAACCTCTGCCTAACCC
CAGCTAGGGACATCTGGACTAAGCTGTAAGGAAGCCCCTGCACACCCTGGACAGGAACCCTTGTCCTGACCATCCACACAAGCCTCAGTCTCCC
TATACGAATCCTGTCTTGACCTCAGACAGGAACACCTTTCTACTCACACTGCTGCAGTCCTGTCCTCCAGGTAGCATTTTAAAAACACCTCACT
CACAATAATCTCAGTAGTAAACCATGGACCCAGCTCTCTCTTTACAGAAATCCCTGTCCTCAACCTCTTCAAGGACCCTAACTATGTGTGGAAC
CTCTTGCCTTCATTGTTGACAATGATCCCTGATGTTCTCCCTGCACAACAATCCATGACCTCACACCCTATCCCATAGATCCACAGGAACCCAT
GATCCAACTCTCAACATGAACCTCAGTCTTTAACTTGTATAGAAAACCACTTCCTGACCTTATGGCAGGAATGCCTGTCTTGACCATTTACTTT
AAGCCCTGACCTCACCCTATACAGACACCTATGGCCTCACCTCATACACGACCCCCCCTTCTCACTATATAGGTCGCATTTATTCATGCTGCAC
GAGCTCCTGGGCCTTACCTTGTATTAGCTCCACTACCCTGACCCTCTACAGCAGGGGTTCCCAACCCCCAGGACACGGACTGGTACATGGTCCA
TGGCCTATTAGGAATTGGTCCACACAGCAGGAGGTGGGCAGCAGGCAAGCAAGCATTACCACCTAAGCTCCGCCTCCTGTCAGATCAGCAGTGG
CATTAGATTCTCATAGCAGCACGACCTTATTGTGAACTGTGCATGCAAGCAATCTAGGTTGCGTGCTCCTTATGAGAATCTAACTACAAGCCTG
ATGAGCTGAGGTGGAACAGTTTCATCCCCAAACCATCCCCCACCATCCGTGGAAAGACTGTCTTCCACGAAACCCATCCCTGGTGCCAAAAAGG
TTGCGGACCGCTGCTCTACAGGAAGCCTGACCCTAAAACTGTTCCAGGACTTCTGACAGGTCTCTGCAGAAAGCCTTTGTCCTGAACCTCAACA
CAAATCCTTTACTTTTACAGTAATTTATTTACAGGAATCTCTGTAATTTACAGGAATCTCTGATCTTTATAGGAACTCCTAGCTTTACTCTCTG
GAAATACACTGCTTACCCTGAACAGGAAGCTTAATGTGATTCTCTATAACAGCCTTTTCCCTGGCCTTGTCCCAGAAGCCCCACCTGCACCCTG
TACAGGAACCACTGCCCTGACCCAACAAAGAAACACCGACCTGAATCTCTGAAGGGGCAGTGTCCTAACTACACAGAGGATCTTTGTCCTGAAC
CCGCACAGGAACCTCTATTACCCCCAGCAGGAACCTGTGCCCTGTGCTAGCACAGAAAATCCTGCCCTTACACTCAACAGGAACCTTTGATTTG
CCTCTTTAGAGTAATCCAAAGAATGAACCAGTATAAAAAACCTCTACACTGTACAAGAACCTCTGTTCTGTACTTGTTAAACAACCCATAATCA
GAATCACCATGAATACCTGCCTGGAGACTGTACAGGAACCCCTGACCTAACCCCCAATGGAATCACCTGATCTGTCTGTCTCTAGTAACACAGC
TCTGATCCTGGACAGAAATACCTGGCCTGAGTACGCTGTGATCCTCTGGCCTCCCCGTCTACAGAAAACCACTCTCCTGGCCTCAAAAGGAACA
CCTAACCTTAATCTGTATGGGAAAAATGCCTTCACAGTTTTCCCTCTCACTTTACAATGTCTCCAACCTTTGTATTTCCCCTCCAAACCTCCCT
TACCAGCTAACAGAATCATTCTCCTGACCCCAGACAGGAAACACTAACCTGACCCAGAGATGCTCCPACCCCTACACAGTAAGAGTCCCTGTCT
CAATGCTTTAAAGAGAAATTTCTATAGCAGTGCTCTCCAATAGAAATATGTGAGTCACACGCGTAATTTTATATTTTCAATGTAGCCACATTAA
AAAGAGAAAAAAAAGATAAAATTATTTTCAATAACATCAATATACTTAACTTTACCCAATATATTCAAATATTATTTTAACATGTAATCAATAT
AAAAATTACTGAGCTATTTTAAACTTTTTATACTGAGTTTTGTGATCTGGTGTACATTTCACATTTATAGCACATCATCTCAAGTTAGACTGGC
CACATTTCAAGAGCAGTAQCCACACGTGGCTGGTAGCTACCATATAGGACACCATAGGTCTGTGGAGTCTACGGAACCCTATAGCAGACACTGC
TATAACCTCATTGAGACCTGTTCCTGTTCTTCCTGAACTCACTCTACCTATTCCAGCCTCCTCTGCAGATAGATGTGGGCACATCACAGAGTTC
TGACCAATGGAGTATGGGCAAAAGTGACATATACCAGTTCCAGGCCTGACCCATAAAAATCTCTCATGCAATCCTCCATTTCCTCTCTTTCCCT
GTCTACCAACTGAAAACCAACTAACTCCAAGGACCTAGAGCAGGACAGAGTCACAAAATAGAGTTTGCCTGGGTCCATGAATGAACAGTAGTAA
ATATTCAGATTGGCTTTTGAATGAGCAAGATAAGATCATTTATTCGTTAAGCCCCTGAAATTCTGGGTTACAGCAGCTAGGATAATTTAGCCTA
ATTAATAATAATCTCTCCCCCTCTCTCTCTCTCCTTTTTTTTTTTTTTTTTTTTTTTTGAGATGGAGTCGCCCACGCTGGAGTGCAGTGGTGCA
ATCTTGGTTCACTGCAACCTCCGCCTCCCAGATTCTCCTGCCTCAGCCTCCTGAGTAGCTGGGATTACAGGTGTATTAGCCCGGCTAATTTTTG
TAGTTTTGGTAAAGATGGGATTTCACCATGTTGGCCAAGCTGGTCTCAAATTCCTGACCTCAAATTATCTGCCCACCTAGGCCTCCCAAAGTGC
TGAGATTACAGGCGTGAGCCACCACGCCTGGCCAGTAATTTTTTTTTTTTTTTTTTGAGACAGGATCTCACTCTATTACCCAGGATGGAGTACA
GTGGCACAATCTCGCCTCACTGCAACCTCTGCCTCCCCTCCCAGCCTCAACCGATCCTCACACCTCAGCCTCCCGAGTAGCTGGGACTACAGAT
GCACACCACCACACCCAGCTAATTTTTGTGTTTTTTATTGACATGCGGTTTTGTAATGTTGCCCAGCTGGTCTCAAACTCCTGAGCTCAAGCAA
TCTGCCCTCCTCTGCCTCCCAAGTGCTGCGATTACAAGTGTAAATCCATTCTGCCTGGCCAATAATAATCTCTTCACCGAAATCTAGACACCTG
ATCTCACTGTCTATATGAACACAAGCTCTGAACCTATACAGAAACACCTGTGACCCAGTACTGGCCCACCTGATCTCTTTGTAGGAAGCTGTCA
TAACATAGGTCTAAGAAGCCCCATCCTGGCCACGAATGGTGGCTCACACCTGTAATCCCAGCACTTTGGCAAGCTGAAGCAGGCAGATTGCTTG
AGTCCAGGAGTTTGAGACCAGCTTGGGCAACATGGCGAGACCCCATCTCTATAAAGAAAAAAAAATTAGGCACGCATGGTGGCATGCACTTGTA
GTCCCAGCTACTTGGGAGGCTGAGGTAGGAAGACAGCTTGAGCCCAGAGGTTGAAGCAGCAATATAGCCCTGTTCACACCACTGCACTCCAGCC
TAGC~CAACAGAGTGAGACCCTGTCCCCTGTATCAAAAAACAAAAACAAAAAAACAAAAAAACAAAACCTAATCCTTAACTTGCACAGGAACAG
GAACCCATAGTCTGGAGCTCTCTAGAAACCCCTGCTTGATTCAGTAAAGGTACAATATAGAAACCATACGAATCCCTGTCCTCATTCTGTCCAG
GAACATTTGGAATGACCCACCAGAGGTACACTAGAAGTGACCTGACCCTGCCCATGAATCCCTGTCCTCACACACCTCACACAAAAGGAATACC
TCTCCTTAGGAAAAAACGCCCCTACCCTCAATCCTGCAAAAGAATACCAGCCACATAGACACCAACGCCTGACACTACAGCACAGATCCGCAAA
CTATGGCATTCA~GCCAAAGCCAGACTGTGGGCTGTTTTACATTTTTAAAGGGTTGTTTTTTAAAAAGAGAAAGAGAATAAAATGCCACTGAGA
CCGTATGTGGCCTGCAAAGCCTAAGATGTTTACTCTCTGTCCCTTTAAAAAGTTTCCTCACCCCTGCTCTTCAGAAACCTCTCTTCTGATCCAA
CAGAAAAATGGGCCTTGACCCTTTGCAAGACTCCCTACTCTATCAGGCTGCAGGTATCAGAACTCTAGAATTTTTTTGTAGTGTGTTATCATTG
TATGTGTTTTCTTTGGTGAGTGTAGGGGTTTAAAGGTCTTAGGTCTCCAACTCTTTTGCGCTGAGACAAAAGATAGGTTCACGTAGGAGGGGTG
AGAGGGAACAGCCGGCAGGACATCTTCAAGAGGATCACAGCGCAAAGCTACTGGGCGGTGAGACCGTCAATCTGTCAGTCTGATCCAGAACCCC
CGGACTGCTTATCTCTCCGGTCCCAGCTTATAGCCCGGTAGGAAATGGTGGAGAATGAGACAGGGAGATTAGGAGGACAAACCGGCCTCCTGCA
GAGCTGACATTTAGACATGGAGTGGGAGTTGCGGGGGAGACGGGGTGCCTAGGGACAGCTCCCCACACGCACACACACTTTCTATTACTGCTTG
GGAGCAGTGGTTACCCGTTGGAGGGGGCAGATAGGGAGAGCTCCTCCCCATGAGAACTCCCATCCTCAAGGCCATTCTGGCCCTGGGCACTATT
ATTATCACAACTTTCCAGATGAGGAAACGGAGCACAGAACTGTTCAGCAACTTGCCCAAAGTCACATGGTTAAGTCCCGTAACCTCAGTTTCCT
TATCTGCAAAGCAGCCTTAATAATGGGAACTACCTCATAGAATTGTCGTTTACAACGATGCCTGTCACATAATACTTGCTATCATTACCATTCC
CTGCCCCCACTAAGACTCCTTCCACTCAGAATATTGCGGCAGAATATTAATAACTAATATTCGGCCAGTGTTTCCTGTGTACCACCATAGTGAA
TATTAACTCGGAAAATCCTCACATTTAGCATCGTCACCACGCCTATTTTGGCCGCCACCACCAACGTCCAGTGGCAACCTCGCTCTCGGCCCAG
CCACAACCAGCCCGCCCGCGCTACGTCATCACGGCGCGTCGCGCGGGAAGTCGCGGGGAAAAGGTAAGATGCCCCTTCCTAATTCCCTCGCTTC
CTTGCTTGCTCTCCAGACACCGCCGACCCGAAGGGCTCCTAAGTGACTGGACAGGGACGGAGGGGCGGCGGCTATGGCATCCGGGACGACAGCG
ACGATAGCCGAGGCAAGAAAAAGCTTGCACCGCAGCCCTGTCGGGAACCGCCGGCGCCTCCCTTTTACGTAGGCGCAGCACAGCTGGCGTAGCA
CGCCGACGCACCGCCCCGTCGCCTGTGAGCTCGCGGTGTGGCTCAGCCCAGCTGGCGTAGCACGCCGACGCACCGCCCCGCCGCTTGTGAGCTC
GCGAGAGGTGTGGCTCGCGCGAGGGGCGCGGCCTTGAGGCACGGTCCCCTCTGGAGGCGGGATCTGGGCGGGCAGTCGAGAGACGAGGCCCAAT
GGAAAAGAAAACCCTGAGCGCTCGCGTCCCCACACGTCGCCAGAAACTTGGTGGAGCGAGCCAAGGGCCGAGTTTGAGAATGGGGCTGCGTCCA
ATGAGTGGAGCGGTCAGTACAGCGCGTCGACGGCGGAGGCGGGAGGGGGCGGAGGCCGACCGAAGGCGGCTAGGGACTGGCTGATAGAGGGTTA
GCGAAACGTTAGCGGTCGGGTCTGGGCAGGGGGTGGAGCTGGTTGAAGGAACGGGGCAGTCCCCTGAGGAGCGGGGCTGGTTGAAACGCTAGGG
GCGGGATCTaGCQGAGTGGAAGGTACCGGTCCGGCCCGATAAGGGGTGGACTTAAGTGAATCGTTAAGGGTGGAGTCGAAGCCGGGGTCGGGGC
CGGGGCCGGCTGAGTGAAAGGGTGGGTGATTATCCCGGGAGATAGGCCGAAAGGGCAGGTTCGTGAACGAATGGGGTCTGGGCTGGGCTTAGAG
GGTCTGGGAATGGGGCTTAGGAGAGTAGAAGGGGCGGTCATTGGTTGGAAAGGGCAGGCCCTAACACGGACTGTGTGAGCCCGGGGGCTCATTG
CTCCGTGAAAGGGCAACACCAGGGAAAGAGAA2CGTGTAAAAGGGGTGGAGTCTAGCGGGCCGGGCGGGGCCGGGTAGAATGGCCACCTGAGTG
GAGGGAGAGGCCTGAGAGAGGCGGAGTTTGGAAGGCTGTGGAGAATCAGATCGCGTAAGGAATGGAATCTAATAGAGGGGGCGGAGCCTGCATA
AGGGCAGAGAGGTGCGGTCCTCAGGGCAACTAGCCCCCTCACATACCCACGCTCCTCCCCCCACCCCCAGAACCGCGGCAGGGGCCAATGGCCT
CCTCAACTATQACCTCAACCGGCCAGGATTCCACCACAACCAGGCAGCGAAGAAGTAGGCAGAACCCCCAGTCGCCCCCTCAGGACTCCAGTGA
CACTTCGGTCAGGCCCTAGACCCGCCCTGATGAGGGGGAGAGGGAGCAAGTTCGTCATGCCCTGGACTCTGACCCTTCTCTCTGATTCACAGAA
GCGAAATATTAAAAAGGGAGCCGTTCCCCGCTCTATCCCCAATCTAGCGGAGGTAAAGAAGAAAGGCAAAATGAAGAAGCTCGGCCAAGCAATG
GAAGAAGACCTAATCGTGGGACTGCAAGGGATGGTGAGCAGGGCCTGGCTGCAGTTTAGCCTCGTATGACAATCAAAACCCAAAGGTTCCCCAC
CCTCGACAGTTCCCTCTGACTCAGGGCCTAAATGGATCTGTCTCTCCTTTTTTTTTTCCTCTGCACGATCTGTAGCCTTGAGGCTGAAGCACTG
GCTGGCACTGGCTTGGTGTTGGATGAGCAGTTATGAATTCCATTGCCTCTGGGATGACAGGTGAGGCTGGGTCCTCCAAGCCTGTCTCCATAAC
CACAAAAATACACACACTTAGAGCCCATTAGCAAGAATAAATCTTTTCTTACCCCAGCCTGCATACTGCAT4CCAGATGATTTGGGGAGGTAGG
CTCACACTTGAAAGGGTTCTTTTATGTAGCTAACAAACATTTATTCAGTGCCTATTGTGTGCCAAGAGTTGGTGATACAAATGTACCAAACAAC
AGACAGGGGTCCTTCTCTAGGAGCGGATTGATGAGTAGGAGGGCAGATCATTAAACAGCCAACACCAGTATGAGGTGTTTAATGAAGACAGACT
TAAGACCAGGGGATTCAACGGTGTATAATAGAGGATCCTAAACTAGTATCCGGGCTTCCCAGAACCCTTTATAGAGGAAGAGACGTTTGTACTG
AAACAGAAGGATGAAACCTCCCGTTTGCTTTACTTTTCAAAAAGAATCAGCAAATCCTTTTGGCTATATGTTCAAAATATATTCACCATCTGAC
CTCTCCTCACCACCTCCACTGCCACCATCATCATCACTCACCTAGACTTGCAGAGGCCTCCTCCCTGGTCCCCTACTTCCAACCTCACCTCTAT
TATCTGTTCATGCACCAACCTAAAAAAATATGTATGAGTTGATGTCTTTGTTAAAATACTTTAAGGAGGCCAGGCATGGTGGCTCACGCCTATA
ATCCCAQCACTTTGGGGGGGCCAAUGCAGGTGCATCACTTGAGGTCAGGAGTTCGAGACCGGTCTGACCAACGCGATCAAACACTGTCTCTACT
AAAAATACAAAAATTAGTTGGGCGTGCTGCCACACACCTGTAATCCTAGCTACTTGGGAGGCTGAGGCAGCAGAATCGCTTGATCTGGGAGGCG
GAGGTTGCAGTGAGCCGAGATTGTGCCATTGCACCCCAGCCTGGCCAACAAGAGCAAAACTCTGTCTCAAAAAGTAAAAATAAAAAAAAAAAAA
GTACTTTAAGGGCAAACTGTTGCCCTCAAGGTAAAGTCCAAATTTTTCATCCTGGCCTCAAAGCTTCCACATGATCTGGCTCCACATCACTTTG
TGACCTCATCAGCCAATCCTTCTCGC
HUMAN SEQUENCE—mRNA
GCAAAGGCCAAGGCCAGCCAGGACACCCCCTGGGATCACACTGAGCTTGCCACATCCCCAAGGCGGCCGAACCCTCCGCAACCACCAGCCCAGG
TTAATCCCCAGAGGCTCCATGGAGTTCCCTGGCCTGGGGTCCCTGGGGACCTCAGAGCCCCTCCCCCAGTTTGTGGATCCTGCTCTGGTGTCCT
CCAC)CCAGAATCACGGGTTTTCTTCCCCTCTGGGCCTGAGGGCTTGGATGCAGCAGCTTCCTCCACTGCCCCGAGCACAGCCACCGCTGCAGC
TGCGGCACTGGCCTACTACAGGGACGCTGAGGCCTACAGACACTCCCCAGTCTTTCAGGTGTACCCATTGCTCAACTGTATGGAGGGGATCCCA
GGGGGCTCACCATATGCCGGCTGGGCCTACGGCAAGACGGGGCTCTACCCTGCCTCAACTGTGTGTCCCACCCGCGAGGACTCTCCTCCCCAGG
CCGTGAGATCTGGATGGAAAAGGCAGCACCAGCTTCCTGGAGACTTTGAAGACAGAGCGGCTGAGCCCAGACCTCCTGACCCTCAAGGACCTGC
ACTGCCTTCATCACTCCCTGTCCCCAATAGTGCTTATGGGGGCCCTGACTTTTCCAGTACCTTCTTTTCTCCCACCGGGAGCCCCCTCAATTCA
GCAGCCTATTCCTCTCCCAAGCTTCGTGGAACTCTCCCCCTGCCTCCCTGTGAGGCCAGGGAGTGTGTGAACTGCGGAGCAACAGCCACTCCAC
TGTGGCGGAGGGACAGGACAGGCCACTACCTATGCAACGCCTGCGGCCTCAATCACAAGATGAATGGGCAGAACAGGCCCCTCATCCGGCCCAA
GAAGCGCCTGATTGTCAGTAAACGGGCAGGTACTCAGTGCACCAACTGCCAGACGACCACCACGACACTGTGGCGGAGAAATGCCAGTGGGGAT
CCCGTGTGCAATGCCTGCGGCCTCTACTACAAGCTACACCAGGTGAACCGGCCACTGACCATGCGGAAGGATGGTATTCAGACTCGAAACCGCA
AGGCATCTGGAAAAGGGAAAAAGAAACGGGGCTCCAGTCTGGGAGGCACAGGAGCAGCCGAAGGACCAGCTGGTGGCTTTATGGTGGTGGCTGG
GGGCAGCGGTAGCGGGAATTGTGGGGAGGTGGCTTCAGGCCTGACACTGGGCCCCCCAGGTACTGCCCATCTCTACCAAGGCCTGGGCCCTGTG
GTGCTGTCAGGGCCTGTTAGCCACCTCATGCCTTTCCCTGGACCCCTACTGGGCTCACCCACGGGCTCCTTCCCCACAGGCCCCATGCCCCCCA
CCACCAGCACTACTGTGGTGGCTCCGCTCAGCTCATGAGGGCACAGAGCATGGCCTCCAGAGGAGGGGTGGTGTCCTTCTCCTCTTGTAGCCAG
AATTCTGGACAACCCAAGTCTCTGGGCCCCAGGCACCCCCTGGCTTGAACCTTCAAAGCTTTPGTAAAATAAAACCACCAAAGTCCTG
HUMAN SEQUENCE—CODING
ATGGAGTTCCCTGGCCTGCGGTCCCTGGGGACCTCAGAGCCCCTCCCCCAGTTTGTGGATCCTGCTCTGGTGTCCTCCACACCAGAATCAGGGG
TTTTCTTCCCCTCTGGGCCTGAGGGCTTGGATGCAGCAGCTTCCTCCACTGCCCCGAGCACAGCCACCGCTGCAGCTGCGGCACTGGCCTACTA
CAGGGACGCTGAGGCCTACAGACACTCCCCAGTCTTTCAGGTGTACCCATTGCTCAACTGTATGGAGGGGATCCCAGGGGGCTCACCATATGCC
GGCTGGGCCTACGGCAAGACGGGGCTCTACCCTGCCTCAACTGTGTGTCCCACCCGCGAGGACTCTCCTCCCCAGGCCGTGGAAGATCTGGATG
GAAAAGGCAGCACCAGCTTCCTGGAGACTTTGAAGACAGAGCGGCTGAGCCCAGACCTCCTGACCCTGGGACCTGCACTGCCTTCATCACTCCC
TGTCCCCAATAGTGCTTATGGGGGCCCTGACTTTTCCAGTACCTTCTTTTCTCCCACCGGGAGCCCCCTCAATTCAGCAGCCTATTCCTCTCCC
AAGCTTCGTGGAACTCTCCCCCTGCCTCCCTGTGAGGCCAGGGAGTGTGTGAACTGCGGAGCAACAGCCACTCCACTGTGGCGCAGGGACAGGA
CAGGCCACTACCTATGCAACGCCTGCGGCCTCTATCACAAGATGAATGGGCAGAACAGGCCCCTCATCCGGCCCAAGAAGCGCCTGATTGTCAG
TAAACGGGCAGGTACTCAGTGCACCAACTGCCAGACGACCACCACGACACTGTGGCGGAGAAATGCCAGTGGGGATCCCGTGTGCAATGCCTGC
GGCCTCTACTACAAGCTACACCAGGTGAACCGGCCACTGACCATGCGGAAGGATGGTATTCAGACTCGAAACCGCAAGGCATCTGGAAAAGGGA
AAAAGAAACGGGGCTCCAGTCTGGGAGGCACAGGAGCAGCCGAAGGACCAGCTGGTGGCTTTATGGTGGTGGCTGGGGGCAGCCGTAGCGGGAA
TTGTGGGGAGGTGGCTTCAGGCCTGACACTGGGCCCCCCAGGTACTGCCCATCTCTACCAAGGCCTGGGCCCTGTGGTGCTGTCAGGGCCTGTT
AGCCACCTCATGCCTTTCCCTGGACCCCTACTGGGCTCACCCACGGGCTCCTTCCCCACAGGCCCCATGCCCCCCACCACCAGCACTACTGTGG
TGGCTCCGCTCAGCTCATGA

TABLE 14
MOUSE NOMENCLATURE
ICSGNM Fkbp5
Celera mCG18519
HUMAN NOMENCLATURE
HGNC FKBP5
Celera hCG17659
MOUSE SEQUENCE—GENOMIC
ATAGTGAAACTGTCTCAAAAACAAAACCAGGGCTGGAGAGATAGCTCAGTGGTTAAGAGTATTGACTGCTCTTCCAGAGGTCCTGAGTTCAATT
CCCAGCAACCACATGGTGACTCACAACCATCTGTAATGGGATCTGATGCCCTCTTCTGATGTGTCTGACAGCGACAGTGTACTCACATACATGA
AATAAATAAATAATCCAATTTAAAACAATAACAAAACAAACCCAAACAACAATATGGCCTAAAGTTATAGATTCATTCCTTTGTCTCTGCCCTT
ACCAAGTGTATGTTTTATGAAGACACATTTTCTAGAACAATGCCTGAAGCAGCACACTTGTTTACTGGATTCCCTTCCAGGGACTCAAACTAGG
GCCTCATAAATAAATAAATAAATAAATAAATAAATAAATGGAGTTTTATAATTTTATGTGACTGGATATTTTTCCTGCATGTAAACCAGGGCAT
CCCTGAGACCCCAGATGGCCAGGAAAGGTGGAAGGATCCCCTAGACCTGCAGTTCCAGATGGGTGCTTGGGATTGCATCCGGCTCCTCTGGAAG
AGCAGCCAGTGCTGCTAACCATGCAGTCATCTCTGCAGCCCTGAAGGAAGCTTGAGTTAGGAAGGAGAGCACTGAGAGTACCCATCAGNNNNNN
NNNNNNNNNNNNNNNNNNNNNNNNNNNNNNNNNNNNNNNNNNNNNNNNHNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNACTGCTGAGGCTGAGTTAGGCTAGCAAGTAAGGCTCCGC
AGCTGCAGGGAGACCAGACTCCTGGCAACTGTCCTGATCACCACATCTGTGCTATAGCAGGATCCCCTCCTTCCCCATTAATAAACCAACTGTG
ATTTGGGTTTAGGTTCGGTTTGTTTTTGAGACAGGGTTTCTCTGTGTAGCTGCCAGTCTTCTTATCCAGTGGTTCTCACCTGTTTACCAGTGTA
GTGGGGCTCTGCCTGCTGAGGATCTTTGCAGACATTTTAGAATACATATAAGCAAATACATACACATTTCTTCCAAAAACAGAATCATGTCACA
AATTCATCTACATGTGCCATTATCTTTTCTAAGACGATTTGTTGGTGCCTGGTGTCTGGCACACATCCCTTTAAAGCACGCAGATCTCTGCTGA
ATTTGAGGCCGGCCTATTCTACATAGTGAGTPCCAGGACATCCAGGGCTATGTACAGAGAAACCCTGTGTATTAGTGTATGGGGTGGGGACGGT
TTGGCAGCTTTCTAGTTTTTGTTTGTTTGGCTTGTCAGTATTACAAGGTTGTTTATCTGCAATGGGTAAAGTTTCACTTGTTCCAGGCAGATGG
GGTTTGTAGCTGTCTTAATCCTAAGGGGGACATCGTAATGAAAACAGACATTTGACATTTAGGTGTCAGCCTGTGCAGTTGTAATCAGGTATAA
ACTGTGTCAACTGCAGTCATAATGACACATTCTGATGCATGCCTTCTCATCCGTAACTCTTCAGTGTCTAGGGGGCCAAGTCATCACTCATGCC
ATAGGGACACCCTCGATAACGGCATTCTTCGTAATGCACTTTAATGAAAGCAACAGTGTTCTTTTTCAATAAACATTTACAGATTTCTTCCCTA
GTTAGGTAACTTAAGTGTTTTTTACTGTTAGAGGTAAAATAATCTTCAATTGGACATAGATTTAGCTCATCTTTTTCACAACTGTGACATTCAT
ATAGTATGCATTTCCAAATGTTTGCACTTTCATGATTATTCACTTGAGGAATTGTTTTTTTTTTTTTAAGATTTAATTTATTTATTTATTATAT
GTAAGTACACTGTAGCTGTCTTCAGACACTCCAGAAGAGGGAGTCAGATCTCGTGATCGGATGGTTGTGAACCACCTTGTGGTTGCTGGGACCT
GAACTCCTGACCTTCGGAAGAGCAGTCGGGTGCTCTTACCCACTCAGCCATCTCACCAGCCCGAGGAATTGTTTTTATTTGTGACTGGAGAGAT
TGCTCAGTAATTGAGAGCACTGACTGCTCTTTCAAAGGATCCAGGTTCAGTTCCTAGTGCCCACAACTATCTGTAACTCCAGTCTTAGAGGAAC
TCTCTGGCCTTTGTAGGCACCAGGTATGCATCTCGTATGCATATATACATCAGGCAAACACCTATCCATATAAAATAAAATCATTTTTTAAAAT
AAGAGCATATTTTTAAAAGAAACATTTTTATTTAGCCACACTGTTAATTTAGCTCTACCTTATGCACCCTGTCGATATTTCATTCACAGCGACA
CCTGTACGCCTCCGTTTCTGATCGCGACTTTTCATTCCTTCTGGTGGTTTCCCTTATGAAGTGAAGCATGACTGGTTCTTTGTACACTTAAACC
TTTCCTTCCCTCTGCTAAAGCATTTCATGTTTCCCTCTAAACCCAGCTGCAAACGTCAGACTTTCCACCTGTCACCTTGGCATCTCTATCGACC
CCTCTCCTGCTTCACTTTCAAACPCAQGACGCACTGTTATTCTCTCTCTCCCAATCTTTACTCTTCCCCAGTCCCTCTGAAAGGCACTAGTGAA
CACCCACACTCAGCACTTCATGTCCGTCGTTTCAAAACAACCTTTACCCAAATACTCATTCTTCAGAAATGTCCTCTCTTACTCTAACCACCAT
CTGCAGAAGAGTTTGAAGTTATAGTCACAAAATTACAGACAAAGTACTCAGGGAAGTGAGCAATCACATAATAAAACATCGTGAAAAACACTAC
TAATGTCTAAAAGGTCTCTGTGACACACTAAAATTTGTCCATAGCTGCTGACCAAATAATGACCTAGAAATTCCATCATCAGAAATCTCTCCCA
ACGTAGAGTGAAAGGACAGTGATGGAAAGGAGAGAAGCGGTGGCTCCAGCCACATCTGCTGCTTGCGAGACCGTGCAGTGTGCTCTGACTCCTG
GTGCCTGGATGCTGACTCATCCTGGCCCTCTGGTTGGAGTCAGGCACCTACAGCATTTTAAAAGCTCTGTGGGTGATCCTAATGCTGGCAGTGT
TAAAAAATGACTAAGAAACCTGAACGGTAAGACAGGTCTGACATCTCTCCAACCAGACTTCAGGGCAGAGGGCCAGATTACTGCAGTGTCTGTG
CGGGGAGACCTGTAAGGATGGGAGACCCATCGGGGCTGAGGGAAGACGATAACACCTGCGTACCTCTCTCCTTACTGTAATTCTGAGAAGCAAA
AATACTTTTGTACTTTCCATAGGGAAAATACAGGAGCCACACTAGGAGCAAGAGAAGCCTGACTGGTCTATGGGACCCAACATAGGTCCTGCTC
TTGGCCCCTGAGTTCTCTAGTCAACCCCAAGTCTAGCGCTGCACTTGTAATGCCCTACTCCATGGTCACCCCACCAGGGAAGTCGTTTGCTTCT
CTGTTGGATCCATTGATCTCGTCTGCTTTCTATTCCCCGACTTCTCACAATCAGTGCTGCATGCCTACCTTTTGTTCCTGTTTTCTGGACTATT
AGGGTATTACAGACTTACTGGACCTGAGCTTGTCAPGTCCCTTCAGTGCAGAAAACAGCTTGTGTAGTATGACCTTGAGAAGCAAAAGCTTCAA
CCTGCTTGAAGCCCACGACCGTAAAATAGTAAGAGGTAAAATACTTTATTCACATTTTTCTTTATTGCATAATGTTACCATTAGGTAGTATAAT
ATTTTCTAGGGTTTTTGTTGTTGTTGTTGTTTTGTTTTGCTAGCAATACAACCCTGTGCCTCCTGCTTACTTAATCATTCAGACCAAAATGATG
ACACACACCTTTAATCCTACTCCTCAGGAATCAAGCAGGAGGATCTCTGTGAGTTCTAGGACAGCCTAGGCTACATAGAGAGACCCTATCTCAT
AACAAATAATTCACCTGGCTAAACCGCCCCAGCAAAGCAAAGCTGCACTGTGAGAATGTGTTGGGGGTTCTAGGCAAGGAGCGTCTACTTTATC
TCTTCCCAGGAGAACAGTCCCCTGTTGCTCAACTCTGCTGTGGTGGACATGGAGCTGTCAGCGAAGGGGAGGCTGCTTCCCTTGCCAGCCAGCT
CAGCAGGTCAACCCTCACAACAACGGAGTGACAGACAGAGCCACATTACCTTACATCCAAGCCGACTGAATGAAGAATGAGTCCTCCAAAGCAT
TGTGTATATTGAAGACTAGACTGAGTAGTTTCAGQCTGTGAGGTAGCAGAGCCTCTTTCTTAAACTGTCTCCTTACTGGACCTCTCAGAGCCCT
TGGCTGAGAGGCTGATGGAATCACTCCCAGAAATCCAGCTGAGGATCGAGGAAGGACACTGCAACTGAGGAAGGGAGGAAGACCTCAGAGGGAC
TTGGCACTCAGGACTCGGAAAGGTGACTCCAGCTGGCTGGCTGCCCTTGGCTGGCTGGCCGCTCCGATACATCTGTCTGATCAAGCTGTTTCTG
TTGATTTTTCACTTCTTATAAAAGTATACATGCATACGTGTATGATAAAATATACTGCTTTAGACATTTAAATGTTCCTAAGTGGCATTAAGTA
CACTCCCTCCCAATATTGTGAAACCATCACCGGTCATTCTTTTCATCACCCCAAACAGAAACTCTGTACCTTACAACACCCCCTAGTAACCTTT
AACCTACTTTGATTCTGAGTTTGACTCTGTATGTGTTGTATACATGTCTATGTGTTGGCATGTGTCTGCACTTGTGTACAGGTCTGGGGTCAGC
ATCCAGTGTTGTAAAATGTCTTCGTATTTTATTTATGATTGGTGTGTATGCATGTATATGCCACAGCATTTGTGTAGAGGCCAGAGGATACTTT
TAGGATATGGTTCTTTCCTTCCATGTGGAATCCAGGGATCAAACTCCAGATTTCAGGCATGTGTAGCAAGCGCTTTTACTTTCTAAGCCATCTC
TCCAATCCTGCACCAACTCTAGACTGTCTGGCCAGCAACCCCCTGAGATCCTTCTGTGTCAGCCCATGCTACCCTCACCCAGCATGCACCACTG
TACTGGGCTACAAACTCGGGTCCTCACATGTTCAGCTTGTGCTCTGACACTGAGCAATTACCGTCGAATCCCCAACACGTACACTCTTATTTTC
CCATGATATCCATTCTGATGGGTAGGAACCGGAATCTCTTACAGTGCTTCTTTCCCTTTAACTGGTCACTGTTGATGTGAAACATCTTGTCATT
TTCTGCATGAATGTGTATGTGTATATATATATATTTTGAGAGATTGTGTGTGTGTGTGTCTGTCCAGTCCCACCACCTCAGTATAGATAGGAGA
CATTTCTTTGGGAAAAGTAGCTGAATAATTGTAGTGTGTAATTTCACGGAGCTGTGTAGTTGTTTCCTCTACCTGTGTAGTTAGCTCTTTTTTT
TTTTTTCTTTTTTAAAGTGTATTTCTTGTCTGCCAATAAATAACTATTACTATCTCTAGCTCTAGGTTGGAATGTCTGACAGAGGTCAGACGTT
GAATTTTAAGTGAGGATTTTCCAGTCCATTCTTATAACAAAATGTCAAAATTGAGGTCCACTTAGCATGCCCTGTGTTCTGAGGTTGAGGTATG
CTTCTGTGCTGTGTCTTTTCTCTTGTGTGGAGCTGAAAGCTATAGACAACAAGCCCAGGAGTTGGCGGGGCTTAGAAACACTCGCTGTGGTTCC
TCAGTCCTTCTAACAAAGAAGCTAGGGATGCCGATGGGACACCGTGGACGAAGAGAAGCACCGGTCACATGCACAGACTCTGCAAAACCTCAGG
GTAGCTTCCCACGTGAGAGACCGTAGTTACCTGAAGTTGTCAGTACCTGTTGATGAGACACTGTCTTCTCATGTCTCTGTTGTGTCTCGGGTCA
TTGCCGACTGGTACTTTGGAGGGTGAGTGCCTATCTGCCATGTCCATAACAGGAGCACTGGGTGTTCCCCTTCTGGAGCACTGGGGTGCAGTTT
CCCTCCCTTAGCTCGTCCAGTACAGTACGCTTTATCATGGATTAGTAATAGCCTTAGGTAAACGTGACCAGAGGAAACAAGACTCTGTTAGAAA
AATACTGATATTTCCCTAGCTTTTCTTAAAGCTTTTTAAQAATTGTCTTTTTTTATGTAATACCATAAAGCAAAAAGGCTACTTATTTTGTTTG
TTTACTTATAAAAATTTATACTTCTAGTGACCTACATGAATTTAAGCGGAAATGTTTAGAAAATAAAAGGATTTGGGAAGGGAGTTGCTTTGGT
TTTTGTTTGTTTCTTTGTTTGTTTGTATTTTCTTTGTTTGGCAGCTTAGTTTTTGTTTTTTCAAAACAGTTTCTCTGTGTCTAGCCATGGCTGT
CTTAGAACTTGCTCTGTAGACCAGATTGCACTAGAACTCACAGAAATCTCCCTGCCTCTGCCAACTGAGTGCTGGGATTAAAGWTGTACAGGAT
CACCGACCAACTTAGTTTATTTCTTTATTCCTTAAGATTTAAATGGCTTTGAACCTGGAAAAAAAAAAGGTTGTTTTCTGGTTTTCCAGATTCC
TGGTGCTTCTCTTCCTGTCCACTTGGCGGTTCTGTTCCAAGTCAGTCTCAGATTTGCACGCTCGCATCCTTAAGTTGATCAACTTCCTTGGTCA
AGTTGACACTTAGCAGAACCGACAAATGGGTTGAAGGGAGTTAAGACTTACATGAATAGCAAGCCAAGTTGTTCTTTTCTTCATCTTTATTTTT
AATTTATTTATCTTTTGAGAATTTAATATATGAATACTGATTTTACATCATTTCTCCTTTAATTCCTCCCATATCCCCTTCTCAAATTTGTGCC
TTCTTATTTGTTCATTATTGTTACATGTCTGTGCATTCATACACTTGCCCGTGTCTCTCTGTGTCTGTCTGTGTGTGTGTGTGTGTATGTTTAC
CTATGAATACAGCCTGTTGAAGTCTTACAGTTTTGCTTGTATGGGTTTAGAGAGGGTCTTGTGTAGCTAAGGTTAATGCTCACCCCTCATCCTC
CTGCCTCCACCTCCTGAGTGCTCGTATTCCAGGCAGCATATGCTGCTGTCACCAGCATGGGCCTGGCTGAAAAAAAACTGTTTTTTTCTCTTTT
TTAAAAATTAATTTTGTCTTGTCTGCATGGATGTCTTCGAATCATCTGCATACAGAGGGCAGAACAACCTATCACATCTTCTAGAACTGTGTAA
CGGACAGTCGTGAGCCTCCATGTGGGTGCTGGGTATCATACATCAGTTCTCTGCATGCTCTTAACCATGGAGTCATCTCTCCAGCTTTCCCATT
AAGTATTTTTGGAAGAAATAGCACATAGATAAACTCCGCCTCCCCTTACATCGCCATGAGGCAGTTTGTCTAATTGCTGCTGTCACAGGTTTCG
TTATCGCGCACAGTGCCTGTTACAAGGATAGTTCTTAGATCAGCCCCTTCCACCTTGGAGTTTTTGAGACAGGGTATTCCTGTGACTAGGCTGG
CCTCAAACTAAGAAATCCGCCTGCCTCTGCCTCCCAAGTGCTGGGATTTAAGGCATGCGCCAACACACCCAGCCCTAGATCCAGCTTCTTAAAT
GTTCCTACTCCTGTCTCCTTTTTGCCCAAGATATTCCTAAGCAATCCCAGACATGTGGGCATATAAAATAGACATACAAGGAGAACATTTATTC
ATAATAAGTCATAGTTTATTTTAAAACAATCTTTGCTGTGCAGATATTTTTACTATTTATTCGAGATAAGCAAATATGCACACCAAAAGATGGA
TATGCTTGTTTGTCTACTACAGTTGAGTCCAAAGATAGACTGTGTTTTGATATTACCTGCCGAGAGGTGGCAGTAGGAAATATTAGCTCCTTGT
TGTATGTCCCTAAACCATGCTGTTTCTATAAGAGCAGAAACCGTTGTGCGTGCGTGCAGCAGAAACACTGCGTAGCTCACAATAGCCTTTGGTG
TGAGCTGAGGGGCTGTGAGCAGCACCGTTTAGCATTGCATGATGCAGTGGCATGCATGGTGAATTGCACGTGTGCGCTCAGCCGAGTCCCACAC
TGTGGCATGCCCAGCACTGCCAGCACACCGTGGATTCCTTGTGAATTTATTCTTGTTTGGGTTTGGCGCCAGTCTGTATTCAGAAACCATTGCT
CCTCCTCAGCTGGTCTCCCCGGGTGTGGGCTCACCACTTCCTCTTTACTTATGCTGCACATCACCTGCTAAAGGTTTATATTGCTTTGTTGGAA
AGGCCCTCTCACCTTTTCTGGTCGGTAACCTGTGGGTGAGGCTGGTGAATATTTCTCAAGAACTTTTCACCTACTGCCTTTAATGTCCTCACTG
CTTCTGTCCTGTTGTGTTGCTAGAGGCCCACGTGTCTCTGAAAACCTACCGAACCTTTCCCAAGGATAAGTCGATGAGCAGCTCCCTCCAGATA
GTTAAAGACCATTCCCTGCTGTTTAGGCCCTTCCTATAAGCTCACGCACCAGATATGTGTCACCAAGTCCTAGAAGCTGGGGGTCGGCCCCCCG
GTCTAGTTTCCCTGATTATCTAGTCCAGCATTGTGCTGAGCTAGGTACTTAACTTTGGTTGTTTGTTTGAGAACAGCTTGTTCTAGGCTTTCCC
CTACCCTGAGCCAAACATGTGGACCTTTTATTCTGGTATAAACTGTGCTAGGAAATCCACACAGGGGCTGGGCCCTGGGATTGTGCTGCTCTCT
TCGAAAATCTTTCAGCACAACAAACAGCTCAACCTGAAGAAAACTTAGCTTGTTTGGAAATGAGTTAAAACACTCATATTTTAGTTGCCCAATT
CTCAGTTCTATTAGTAAAGCGCGTTCTTTACAATACTGGGAGCTGTTTTGTGTTTGGTTTGACTGTAACCAGAGCCCCCACCAGCTGACTTGAC
TGAGCCTGGAGTCTGTCCTTACCCTAGTGTGGAATAGCTTTAGAAGTGGCCCACAGTGAGCCGGTTTTGGTAATGTCCTTAACTTTCAGTACTC
GGAACGCTGAGGAGAGAGAAATCTCCAGTTTCAAGCCAGCCTGGGCTACATAAGAACCTGATTCAGAAAATAATTGGCATTGTTATTAATTCTT
CATTTCCCTGCTCAGTCAAGGAGCAGTAGAAGATAATCCCGGCAGTAGACAAGTGTGTTTCGTTTAGCCTATTGATCACTGATATGCAGATTGG
CTTTGCAGGAGAAAAGGTAACAGCCCTCAGGAGGCAGACAGGTGGATCTCTGTTAGTTCAAGGCCAGGCCAGTCGATACATTTGAGTTCAGGAC
ACTCGGTCTCTGTGAAGCAGAGTTTGTATTCTGAGTACACTGCTCTCACTAACACTTTAGACTGCCTGAAGTAGCAGCGAAAGGCAGCCCTCCA
GAGTTCCCTGCTGAAGACTGTAATATGCAAAGCACACATCCCGTGTTCTACAAGACCCTTTATCTACCAGGAGTGCTTCATAAATATCCACATC
CAGCTGACAACAGCTAAGGAAGGGTAAAATGGCAAAACGACTTGCATGGGCATTGTGCATCTGGCTCATGAGACAAACAAGAGACACTAATCAA
ATGGGTGTGGCTCGTTATGGAAACGGACCTGGTTGGTTTGACCAGTGTGTGTGAAGGAGAGTGCCACAACACCAGAGCCCTCTCTTCTCTCTCA
ACAATATCGCTGTAGGAGAAGACTTGTTTTATGGPTCTGAGACGGCAGCTTCTCCCTGTGTTGTCCTGTTAAGGAATATAATTACCGGCAGGCC
TGACTTATACTCCTCTCATCTAGGACCGTGGCTAGACGGGAAATTGAAGAAGTCGGTCATGTTTTATTTCCTTCTCTGATGGCATCAATGCATG
CCCACAGCA4ATGCTTTTQTTATTGTTTCTGTGTTTTAACTCTATTACTCTTATGTAAGCTTAGTTCTCAGCCACTTTTCAGGTGTGTTAAGAA
GTGATACTTTGATTGAACGAAATGCATTTCTCTTGGCAGGTCTTCTACTTACAAAGGACAATGACTACTGATGACGGCACCAGTAACAATGGAG
AGAACCCAGCAGCCACCATGACTGAGCAGGGTGAAGATATCACTACGAAGATACACACAGGAGTATTAAAGGTAAGGCCACAGAAGTGTGAAGC
AGGGAGGTCTCTGTGTGGGAGCTTGTGGGTGCTTTATGCAGCCTGTCTTGTGGACGGGATAGGGTTAAGTAGCAAACACTACAGCTCACTTCCC
ATCTAGCTTCATAGCTGGGCGTAATCTCAGATTATAAATGGTGCAGCTTCCGCTGGGCTGCGTAAATTAACTCACATCTTAGGCCCTGCAGAAC
AAGTTCCAGTTATGCGTTCCCTCAAAAGAACCCCTCCTTGTTTCACAGCGTACATGTAGATTGCAGTTTAATATGCCATCTTAGTGTGCTAAAG
CTATGCAGTGACTGCAAAACAGTCTGTAGAGAAAAAGGAGAGGACTTAGTTTTTATATACAAGTACTGTTTCTATGTGTCATGATGTAACGACA
CAGAATATATATTTGGATCATCTTCCCTATTAAAGTGTGTGTGTCTGATGCATTTGTGTACACATATGATGTATACTTGCTCCTCTGTCAGGAT
ATCTTCCTCAGTCACTTTTCTACCTTATTTCTCTGTGAGACAGAGTCTGTCACCAAACCTAGAAGGAAGCCTGTTTGAGTAAGACTCGGTACCC
AGCAAGCCTCTGGGATCCGTTTGTCTCTGTCCTCCAGCACTGGGGTTGGAGACACACTCTTTCATGATTATGGTTTTACATGGGTCTAAGGAAT
CTAAGCTCGGATCCTCCTTGGACCAGCTCCATAGACATTTTTTTAATTTGCGTCTTAGCAACAGTGTTATGAGATAGTACCTTTTGATCATGGT
GGGACTGGGTATGGTTCCTAGGGTCTCACTAACTTGCCAGGCTGGCCTTGAATTACAGTGTAACCCAGCCAGGTCTTCAACCTGCCATCCTCCT
CCCTTAGTCTCCTGAGTAGTTGGGATTACAGGATTGTGTCCCCACGATGGGCCAGGTTCTCGTCTGTGGTTCTCTTCTGAATCTCCATGCATTC
CTCTTACCTTCCTCTGTGCTGTCAGTCCATTGAGATCTCTTTTTCCTCCAGCTTCTTAGCCTGGTAGCTGTAATTCCTCACCCAGAGCTCCTGG
AAGCTTCTCAAGACAGTGGAGATTCTTCAGGCTGTCCGCAGCGTCTCCTACCTCATCTTTTTCAGTGTGCTGTGTTCAGAGAACTGGTCTCCCA
GGTCCTCAGACCCTGTGTATTCTGTAACCAAGTACTGACTGATTCATGAGGGAGAAGTTTATTGTGGCTCACAGTGCTGTAGTCTCAGAACTCC
AGGAACAGAGCATTCCTGCATCACCTTGCTTCTAGTAAGGACTCATTATAGAGTGAAAGCAGAAGGCAGGCAGGCACGCAQGCAGCAACATCTC
AGAGGCACAAGGACACGTGGACAGCTTTGTACAACTTGTGCGGTGAGCCTAGTCCCTCCACAGCGAGCTCACAGAGGCAAGAGCTTGATTCCAC
CTAACTTCCCTCCTAAAGGCCTCGTATCCCAGCACTGTCCTGGTAGCAGTTGATCCAGTGTAAGCTTTTCAGCGGACAGCTCATATTCAACCCT
CACTCAGACCACAGTATCCCTCCATCGCTGCCGAGCCTGCTCAGAGCTCTTGGATAGGCAGTATGCTATAGTCTCCTTTGTCCTAGGGAGTAAG
TCTAAGAACAACAACATTTAGGTTTATTATAACTGTGTCGTTTCAACCTCTTTAGGTTATGACACCTGTTACTCTCTAGAACCTTCAAGTCACC
TCAGGGCCTTTGGTAAAGAGTCAGTATGTTTCACAGCTGTGGCCCGGAGATTTGTCAGCCTCCCTGGGTTAAGGCTTCTACTTATAAAATGTGT
AATAATAATAATAATAATAAAAGTTGGTTTTACATTTTTCATATAAATTAAAACTAGATTTTTATCTTTTAGGCAAGTTAAGATTTAASCACAA
GTTTCATCTTGCTGCCAACATAGACACACACATAAGAGGGCACTATCACGTGTPAGCTGCTGTCTTCTGCACCACTGAAATGCTCCCATTCGCA
CAGATGATTGACAGACCGATTGCACCACCACACGCCTGGGTTACATTTTGTTTTCCAAGAATTGACCAACTGCATTAGTATCTTGGCTCTCCCA
AGAATAAAAGAAGTAGTCTGGATTTTACAGTGGATAAATGTTTTTCATTTTGAAGTTCTTTTCTTCTTTTTTTTCCCTTTTCCTTTTTCTTTAC
TACAAAACAAGCTACTTCGCATCGTCTAAGTCTATATTTTACACATTATTCAAATAATGGATGTTTAAACTGTTACATCAGACCATACAGCAGA
GCGTTTGGACCCTATCTTTAGCAAAGGAGGGAAGTAAATATGCCCCCTTGACTCTTTGCTGTTTAGGAAGTCCAGCAGTCCTTTGGCTAGTTCA
GTTTCTGTACCTTTGCCTGCTCATCAGTCTGTQTTTTGAGAGATTTTACCATCTTGGCTTTCAACCAACCAAGTGAGGTTTACTCCCTTTTTCT
CTTTTGCCTTGGTGGAGATACGGTTTCACAGGATAATCTAGCTGGCCTGGAACTTACTGTGTACACCAGGCTGTCTCCGCAGTGATCCTTTTGC
CTTCCTCTGGCTTTCTAGTTTGAGAGACTAAATATTGTGGTGAGCTGTAATAATAGTCATAACACAAGGGTTAGTGAAAGGCAGTTTTCTATAG
AGCAGGACAAAGAAAGCTGTGAGGAATGGCGGTGAAACTGCCCATCTGTCTGGGTGCTGGGGAGTAGTCCTTGTSGCCTTTGTATCCTTTTACA
AGTACAAAGCTCTACACTTTTTTTCTTATGTTTGTCTTTCAGATTGTCAAAAGAGTGGGGACTAGTGACGAGGCCCCAATGTTTGGTGACAAAG
TTTATGTCCACTACAAAGGCATCTTGTCAGATGGAAAGAAGTTTCATTCCAGTCATGACAGAAAGAAGCCATTTGCCTTTAGCCTTGGCCAAGG
TAAGAGGACAATATTTTCAGGGTTTGTGTAGGTTTGATTCTGTTTTTATTTTTTATTTATTTTTTATTTTTATTTGTTTTTTAAGAGTTATTTA
TTTATAAATACCTAATAAAAATGAAAAAAAAAAAGAAAGTTCTAGAGATAGATGTGCAAAGAGAAGCCAGGCAACTGTTGGTCGCGACTGTGGA
AATAAGAGCTAGGTTGTAGCTGACAGTAACCCATTATGAATATACTTCATATAACTGGCCATAAGACTGGATAAGATGGGTTATTTCATCTTAC
ATATATCTTATTATTACCTTAAAAAATGGGCAGATGTAAACCTAATTCTGTCCACATATACTTGGAAAAAAAAAGATATTGTAAATAAGAGTAA
AAAAAAAAAAGAGTTATTTATTTATTATATGTAAGTACACTGTAGTTGTCTTCAGACACCCCAGAAGACGGTCTCAGATCTCATTATAGGTCGT
TGTQAGCCACCATGTGGTTGCTGGGATTTGAACTCGGGACCTGCGGAAGAGCAGTCAATGCTCTTAACCACTGAGCCATGTCACCAGCCCCATG
ATTCTGTTTTTATATTTCACAATAAACATGGCAAACATGGTCCATGGATCCCTAGGTGTCTCTGTGGTCCTTTTGCGACCATAGTGTAACTTCT
TTCTGTAGCAGTGTGAAGGTGATGCCGCTTTCACTTGCTCACGCTCACACTCGCAACCTAAAATCTGCAGAGGCACAAACTGCAGGGACCCTCG
TGGGCATCAGTGACACTCATCTCTGGAGCCCATTGTGTTCTCACTGCCTCTCCTGTTGGCTGTCATCTCAGTGCTTCCTAAGTCGCCCTCAGGA
AGCCTGCTACACTTTCAGTTGGGAACTGAGCTAGCTCAGTGTTGCCGTGAACACCATTCTTCCTTGCAAAGGCAGACGATGACTGTTCAGACTT
GAGAGGTATTTCCTGAAGGAGAATGAGATAAACCTGTCACTTCAAGGAAAACACCCGACACATCTTGTTGCCGTTGCAGCTTCAGGTGGAAAAC
CAGTTTTAAAATATTTGTGGCTGTCCCATGCTAACAGCTTCCTTGAGCCGTAAGACTTCTCTGTGAGAGCGGTGGGTACTAATGAGTGTGACTT
GACTTCTGGACAGTGAGGTTCGTCACTGTTAGAGTGTCTGCCTAACTCAGTTAGCCAGTATTTTCCCAGGGACCAGTGCAATACATACACCGTC
AAGTCTGCACAAGCAGTCCATTCAGGCTAGACCAGTGCTGTAGGGGCTGTAGTGTGAGAAGCCCTTTGATCCAGATGACCTGCTGAACCTCTTT
TGATAGTCATCTAGGAATTCTGTCACGCTGCCCTCCTGCCTTTTCCCAGGTCCTTGTGACTTTCTTCTWTTCTACATCTTCAGTCAGAAGCAAG
CAAATCACTTCCTCTTGGCTTATATCAAGTCAAACATTTCACTTTTGTAAAATTAAAAATCACAGCTGAGGACTGACTGGGGTGGTGTTCTGTA
CTGAGAGTGCTTCCCTGGCATCCACACAGTGACTTTGATCCTCAGGTGCAGTTTCCCACTCTAATGCCAGCACTTGGCAGATTGGAAGCAGTGG
CGTCAGAAGCACAGAGGTGTTCAGTTGACCACAGAACAGAGAAACATAAGCCAGCATAATGGCACACACCTATCTTCTCAGCAGTTGGGAGGTG
TCCTAGTTACAGTTTCTATTGCTGTGATGAAACACCATGACCCAAGGCAAQTTCGGCAACAAAAGGTTTATTTGGCTTACACTTCCACACCACT
GTTCATCATTGAAAGAAGTCAGGACACGAACTCCAACAGGGCAGGAAGCTGCAGGCAGAAGCTGATGCAGAGGTCAGGGACGAGTGCTGCTTAC
TGGTTTGCTCATCATGGCTTGCTCAGCCAGCTTTCTTATAGAACCCAGGATCACTAGCCCAGGGATGCCAGCATCCACAATGATTCTCCCCAAT
TAACCACTAATTAAGAAAATGCCTACAGCTAGATTTACCGATGCATTTTCCTGTTGAGTGCTCTCCTCAACTTCATATAAACTAACTACCACAG
GAGGCAAAGGAGAGGGCCAAGTTCAAAGCTGTGTTGTATTTCACAGCAAGACCCTCTCTCAGCAAAAGAGAAAAAAAATTAGCTACGCCATTTA
GATTTTATATTAAATATTATAACAGTTGCTGCTTCATCAAAATAAAAAAAAAAAAAAGGAAGAAGTATAAATACTGTCTCGTGAGCTCTCTAGT
CCCTTATACTATATGTCTCCCAAAGGAGTATATACAAATATTCCAAAGAGACGTTCCAGGCTAATTTTCGGATTCTAGTATGACTTCAGTGTTT
TGTTTTGTTTTAAGATATAGTCTTCCCTGCCTATCTTCGAACTCACCATGTAGAGTAGGCTGGCCTCGAACTCAAAAGAGGTCTACCTACATCT
CCTGAGTGCTCCGGCTAAAGATATGCAATACCACACCTGGCCTCATTTTAGTATTTTAAATAGGACTATTATGAGCCTCACTATAAAAACTTTA
TTGAAAAAACACATATACAATACACCACTGCCCCTCCTCCTGCTGTATGCCCACTGCCTTCCTGTACTGACTGAGCTCATCCCCTACACTGTAA
ATGACTGTTCTTACTACCAACCGCTGTGCAATGTTGGGPGAAGATAGCACACACCACTATGTACATCAGCCCAGCATAGTGCACACCTGGGTGT
TATTGTTATTACAGCAAAAGATTACTGGTGGCTTTAAAGTTTTTACTGCAGTCACAGTAAAAAACGATTGTAACTTCTTZTGTCGTAACTCTCC
TTACTGAGCAGTCATATAAACCTTTTGAAACAATGCTTAGCCAGTCTTTTTTTTTTTGTCTGAGGAACTTGGGAAAGAGAAACAGAATCAGTCT
AAGACCTGGTTGTTAGCGCAGAGGACTGTGTCCCAGGATAGAAACAGGATAGTTCCCCTCCTGTGCTTACGTTACTTTTGTTTGTCGTCCCATT
GTCCAAATTGATTTTTTTTCTCTGAAGCACCCTTCCTTGATTGTGAAGTACTAAATGATAGAAAACTTCAATGTCCACTTTTCCTTTTCTAAGG
ATGTCTCTTATCTCCCTTGCTGAAAATTACTGAGTATACACTTTTAAATATATGCTTACATTTAAACTCTCAGTATGTTTAGAGTCTGACAGCT
TGAAAGTGTTTTGTTTGTACCTGAGGAGGCACAGCTACAGAGGGTCACAGTGTCTGCTCACTTGTQCACAGTGAGTCAGCCAGGACTCACACCC
CAGGTTGTGTTCCTGAAGTTTCTTCATTCCCAAGGTGTGATACAGTCCAGTCCAGGTCTAAGTACAGCCGCAGCCTGCTCACACCAGCTGCTTT
CCTTTTTGCAGTGATGCATTGCTGTTTGTAGGACTTATAGACTCAGAATGCATGTTCTTGCTTGATGATATTGTGAAGTTAACGTGTACTTTAA
AAGAAGTGTCATACTTTCTCATAATACTAAGCCTAAAAGACATTTTACATCACAGTTAATACACTTATTAAAAGTACTGAAACTTGCTGATGCT
GGCACACGCCTTTAATCCTCCACATAATCCACCATGAGGCCATATCACATTTTGTTTCAGTCAGGTGCTGAAGGACACTTGAGTTGCTTCACCT
CTGCATCACGGTGGCTCACACTTGCTGTGACCGTGTGTATAAACATGAGACCCTCTACACAGTTCTTTTTGATCACCATCCAGAAGTGGACTGC
TGGATCATAGCTGAGTCTTGTGTTTAAATTCCTTTTTCTCTAACTTTTATTTATTTATTTATTTATTTATTTATTCTTATTTTATGTGCATCAG
TGTTTTGCCTGCTTGGTGGTAGCTGGGGATTAAAACTGGGGTCCTGAGGGAAGCAGCCTGTGCTCTTAACTGTTGAGCCGTCTCTCCAGCTCTA
ATATTTCTTTTGTGTTTCTTTTGAGACAGGGTTTCTCTGTGTAGCCCTGGCTGTCCTGGAACTCACTCTGTAGACCAGGCTGGCCTTGAACTCA
CAGATCCTCCTGCTTCTGCCTCCCGAGTATTGGGATTAAAGCCATGCTCCATCACATGGACCATATTACTCAGTCTTAAAGGAGAGGAAAACTC
TGGCACATGCTATATCAGTCATGAGCCCCAATCCTGAACCCCAAAGACATCCTACTAAATGAAATAATCCAACCCAAAAATGAAAGCTGTGTGC
CTCCACTTGTATTACATGTCTGTAGAAAAAAGTCTTTGCTGCTGCCTGAGAGGAGGAGGAGGAAATAAGGTGTGTGATCACGTATAAAGTTTCA
GACGAGTAAACCAGAACGTTCTGGAGATAGGTCGCACAGCGTATACATGTAGTCACATCAAGTGCACACTGAAAAAATGCTTAAGATGATAAAC
TTTGTTTAACCCCAGCCAAACATGTTTGAAGTCAGAGGAGTTACACAGPAAAAAAAACACTAGTCAGTTATTAATAAAGTAGTTTTTATTATCA
AGGGAAGTATTAGCATATTTAAACTATATGTTTTTTAAAGTATTGTGTGGGTTTTTTAACCACARAATGTATATATCACTACAGAAGAAAGGAA
AAAATATAGTCCATTGTTTTTATCCTGGGCTAGTGTACATTTTACTTACATATTTATTTGTGTGTGTTGTGCAGATGTGTGTCATGTTTGTACG
CATGTGTGTCGACTTCAGCCATGGGAGCTGTCCATGCTCCTTTTGAGGAAGGAGTTCTCTTTCCACCTAGGACTTCCCCATTCGGCGCCACTGG
CTGTCCCGAGCCTTGGGGTCCTCCTCTCTCTGCTTCCCAGTGCTGGCCTTAGCAGCACTGCTGCTGGGCCTGGCTTTAAGTGGAGCTGGGACTC
TAACTCAGCGCCTTACACTTGACCGCTGAGCTGTTTGCCAACATTGCCTTTAAACTCAGATTGCCTGTGTCTTTGTAAGTACAAGAGGTATGAA
TATGTATTCTTTGATACTTTGTTACAAAAAGAATAGAACTAAAATGGAAACATTGTGTTTGAGGTACTAAAATTGAGGTCCAGACCCAGATCAA
ATGTAGGACTCAATTTTTAAAAAATTTATTTTTGGAATATTCTGTCTCCAAGATAAAGATCCTGAAGATGAAGGCTCTTTGCAACATTGATGCT
TGTGGCCCACTGGTTCTCCTTCTGGGGACTAGACCTACAGCAGTCCTGCCATAGTGTTTAACCAGCTCCAGACTACCTTTTGCTCTAGTTTTCT
TTGCACATAGCCTGAGTGATACAACTAGTTCCCATGAGCTGAGCGATATTTCACACACTGTGCCCATGAGTGCCCTTGGCATTGGCCCTCTCCA
GCCACAGACCGCCAGGAGCACACTACAGTTGAACAATCTAGGTTCAGCCCCTTCCAGAAGGATGCATACAGTGGGCAGTACAGGGTTGGAGAAT
TGCATCTCACAAAGGATGTTGCGGTGCATGTCAGAAAGGATGGTGGCTACGTGTCAGGATGTTGCGGTCCATCTCAGATAGGATGGTAGGTACG
TGTCAGGATGTTGGGGTGCGTGTCAGAAAGGATGGTGGGTACGTGTCAGGATGTTGGGGTGCATGTCACAAAGGATAGTGGGTACATGTCAGGA
TGGTGGGGTGTGTGGCACAAAGAATGTTGGTAGTCTTGTGGGCTCTCGTGTGGTCTGCCAGAGTTCTGAAGAAGCAGAAGTTTGCTCTGGAGTA
GAAAGGATATTGAGTAATCCTGTGCTGGAGAATGAAGACTTGTGCCTAGAGCAGAGTGAGACGATATTAATCTATGGTGAGGTGCAACAGTTGC
CCAGAGCAGCCAGGAGACGGGGCTCTTTGATATTTTCTACGGTGTACAGTGACCTTATCTTTTATCCAAGCTTAGATGATAATTTGAAGTGTTC
TTGTTTTGTCTGAGTTTCTGATGGTTATGCAGTGACCTTGTCTGCTGTTGGTGTTTTATAAGCTTGACTGTGTGCAGTCACAGACTGATGTGGC
CTTGCTCCGAGCCCAGGCTCCCCCCAGACAACGGAGCCTGCAGCGAGTGTCAGCACCTGGCCATGGCCAAGGACTATCCTTTATTTCTCTTAGG
AAGAGTCTTTAACCTAACTACCCATGTATACTTTTTCCAAGTAGTTTGTTTCAGTAGACTTAGAAAACATCAAGATTAATGGTAAAAAGGAGGA
GGAGCTGTCTGAGAGACTTGTAGGATACPCATACAGACTTGTTACGAGCAGTGCTGAAAGCATTCTTGAAATGCCATTGACTTAGAGGTCGATA
AACAGTTAAAGTAAGTCGCCTTCAGAGCCCGTCACCAGGAGAGCCTGCATATTGATCACCTGCATTGTTCGGATCACGTTAAAGGAGTTTTTCT
GTAACCAGGATTGGCCATACTCACTGACAGAGCCTTGGCCACTAACATACTATGATTAATAACCTCGCCATATTTTAATTTTAAAAAATGAAGT
ATATGTTGTTTTGCATGATTCCTCGCTTTTAGTTTAAATTAATGTATCAGCCAGACCTGGTCCTATCAGTTACAGGCTTTCACTATACAAAGAA
CGTTACACACACGATTGTTTCTGGACTTTGATGGTAGCACAGGAAAGCAGCGACCTGGACTAGACCTGCAATACTGTGGTTAATAAAAGGGCAC
GTGTTCAGACAAAGTCAAGAAACGGGCTTTTATAATGTAAACTATGTTGAGAACCTACCGAGCCAAATAATGAATCATCTCAGTTCTAAGAATA
G~CGTCTAAAGGTTTAAAATATAACAAACAAAAATTATCTCTATAATTTAAAGGAAATTAACCAGGAAATTGACAATTTCCTTCCTTCCAAGGA
TTCAGAAGTTTTCAAATGGTGTTCTCTATGTTTCAATGTTTTCTTGGTGTTGCTTTTCAGAAATTTGATCGGGGAACTGGTAAGGTCGTCCCCA
CCTATAATTCCAGCACTAGAGGCGGAGACAGCGGAGAGCAAAGGAGAAGGAGGTCCAGCGTTAGCCTTCCTGACAGAGGAGTTGGAGGTCAGCC
TTCGAGCACATAAGATTGTCTCATTAATCACAGCAGTAACAATGCCAGGCCCTTCTGTAGCACCACTTCTGCACTGCAGGCTCCTGGAAGGGAC
AAGAGAAGGGTGTACTTTTATATTACAGTATCATTTCCTACCAGTTCTTGTGACTTTCACATGCACACATACATTAAGTAAAGTAGTCCATTTT
AAAAGAAGAGTCAGATACATCGGGCTGCTAAGGTGGCTGGGTGACCTCAGCCATCACCCCTGGACCCATGTGAAGATGGAAGGAGAAACCAACT
CTACCAAGTTGTCTTCTGGTCACCTCCACATGTGCACCAGGGCACAGGCACACGTCATACACAGCTCATACATTAATCACACAGGAGTGAGACA
GAGGAGCGCTGGCTCATTTTTTTTTAAAAGATTTATTTATTATTATAAATAAGTCTGTCCCCAGATGTACTAGAAGAGGCCATCAGATCTCATT
ACGGGTGCTTCTGAGCCACCATGTGGTTGCTGGGATTTGAACTCATGACCTTTAGAAGAACAGTCAGTGCTCTTACCCACTGAGCCATCTCACA
GCCCCGGCTCATTTCTTAAGATGACGTAGAATATTACGAGTTGAGACTAGCTGGACTCTGTAATAAGACCTTATCTCAAAAGCCCAAAAGAGAC
GGAGTTATATGTGGTGGTGAATGTGAGGGCAGCAGCTTTTCTTTATTTTACTAAATAATTTATTCTTTACTAAATACCAGACTCTTTGTGTGTA
TATACTGTGCCATGCATTTGGGGTGGGGGCTGTTATTATGTTCTCTGGGTCAGGAAACAGATTAAACTTGCACACCTTTCCAAACCAAACCTAA
GCTTCGAATCTGGTACTTTGAACCCCTACAGTGCATAATCCTCCCTTTAGTAATCAAGAAAGAAAGTCAAGCTATTAAATAAAAAGGAGTTCAG
AATTCAATGATAATACGCTGCCATATATTGAGGTGTTGGTTTGTATTGTTTTGTTTAGGTTTTTCAAGACACGVTCTCACTATGTAGTCCTGGC
TGTCTTGGCACTCTGTATGGACCAGGCTGGCCTTGATCTACCCACCTCTGCCTCCCAAGTGCTGGGATCAGTCCCCTAGAACTGAAGTTACACA
CAGCTGTGAACTGCCACGTGGTTGCTGGGAACTGAACCTGGGTCCTTGCGAAGAGTGGCCAGTGCTCTCCACCGCTGAGCCATGTCTCCAGCCC
CTAAATGAGACATAGTTCATGCTGCACATCATAAGACTATTACTCAAGCATGGTGCACACACCTGTCTGTCATCCAGGAGTCCTAGACCCAGAC
ACCATCTGCCAGAATTCAGAGTTCTAGGCCACCTTGTGCTGCATTATCAGGCTGTCTTAAACTTTCAGAACAAAGACTTGTAAATATTGACACT
GAACAGGTTGCCTGCTACAGGTCCATAAGAGACCACTAAAGCTGAGTTTCTCAAGTTGGCAAGAAAGGAAACTTCCCAAATGTTACAGTTCTTA
TGATTTGGCAGATCTGCGGTTTGTTTGAAGATTTTCTTTGACAGATTTGGTPTTPGTTTTTGTTTTCTTGTTTTCTCTTACGCCAGGTTATCAA
AGCCTGCGACATTGGGGTGTCTACTATGAAGAAAGGCGAGATCTCCCATTTATTATCTAAACCAGAATATGCTTATGGCTCGGCTCGCCACCTC
CAAAAAATTCCATCAAATGCAACTCTCTTTTTTGAGGCAAGTATGTGTAGTGCTGTGCATGTGCGGCCATGACACGGAGCTGCGCTGGCCGCTG
GCACCCTGCTCCAGCTTATTTATTTTGGAGCTTGGCTTCTTGAATTGTTTCCCACACATCCAGGGGGTGTGCCAGTGCTTTCTTAGAAATGTGT
CTGGCACAAAGTGGGCATATCCAAACAAACAAAAGTCACAGATGTGGAAGTAAAAGTTCTGGTCAGTAGAGGAAGTAAGTCATATCCATCCTGA
CACQCTQGAAATGGTGGTGGGGAGTGTAAAGTTGTGAAGGTTTCCGACTGTCTCCTCATATCCATCAGCAGCTGTCAGTCGTCGTCCATTGTTT
ACTGCCCTGAATTAATTACACATAATTGGCCAAACTGCATCTTTATCTAACACGGTGAAAGCACTTCTCAAGTCAGTTCAACCCCAGTTTCTTA
AATCTTAAGGTGGCAAGGTCACAAGTCTGGGGTGGGAAGCGGTTGTCCTCAGAGTCAGCCAAGACTGTTTTGCGCCAGCACCTTGTCCTCCTGG
CCTTTTGCCCACAAGGCAGTTGTCCTACTATTTCCTGTCTGTCTTTGCACGGCAGTCCCACAGGATGCAGTCCTAGACGGTCTCATTACACGGA
GTTCCTTCCAAGAACTGCAGAGTACATCGCTCTCATGTCCCACGTTACTCTTATCTAAGCTCTATAGATAAAGATGTAGCTTAAAGTTTTATAA
AAATAACTGTTAAACTGGCTTTCATCTTTGCATTCTAGATTGAGCTCCTTGATTTCAAAGGTGACGATTTATTTGAAGATTCAGGCGTTATCCG
TAGAATCAAACGGAAAGGCGAGGGATACTCAAACCCAAACCAACGACCAACGGTAAAAGGTGAGTAGTGAGTACTGCCGGGAGCCAGCCTTCCC
TGAGCAACCAGACATGCTTCTAAGAGAAAAAGTCATTAAACTCTCTATAAGAAGAAGAAAATCTATCTAAAACAACTACAATCACTTTCTTCAT
TTTAGCATGTATATAATATTTAGACCAATATTTTATTACACAAGTCTAGTTTGGGTTTAGTTATTTTCTTCTTGCAGTGTTATAGGTAACATGT
CATTCAGGATTGGTTCTAAACAAAGAAGCTCTCAACCAGGAACAGGAAATTGGGATCTAAGGCTGGCTCTGCACCTCTGGGCCTCTCCTGCGCC
CTCACTTCTTCCAACACTGACGCTGTGCTGGGCTTCTTGGTCTCAAAGGCTCTGCGACCCCCAATAACCCACATTGGGAGTCGTGCTTATGAAA
GTCGAAAAGGCTACAGACAGCTGTGACACAGCCACTTAAAAGCTTACTCATAACATGTTTTCATCGTTTATTATGAAAAATTCAAATCTATACA
CCAGGGTTTTTCCTGTGCTGTGGGAACTCCTCCAGAGTCAGTGATTCATAACAACTTGTTTTGTTTTGATTTAGTTTTTGAGACAGGGCATTTG
TTGAGACCAGGAAGTTAATAGGGCCATCCCCTGTCCCACTCTCGTTACCATACTAACTTCTTTTCTAGTCTGGATACAAATTCAAAACCTGGAG
CTGCATTTACCCCTCATGGCTTTATTCTATTCCAGTGAATTAGTCTTCCATCTTTGTTTCCTGTGACTTTGACACTTTTTTGTTTTTGTTTGTT
TGTTTGTTTGTTTTTCGAGACAGGGTTTTTCTGTGTAGCCCTGGCTGTCCTGTAGACCAGGCTGGCCTCAAACTCAGAAATCTGCTGCCTCTGC
CTCCCGAGTGCTGGGATTAAGGCGTGTGCCACCATCGCCCAGCAACTTTGACATTTTTGAAGACAACTTTCCATTTGCTAGTAGAATCCCATAG
TTGTTGTCTTATGCCCACAGGGACTAGATTGAGCTATTAGAAAATGCCCTGTGCGTGGCGCTATGCCCTCTTTAGCTCATCATTATCATCATCA
CCACCATCATCACCATCCCTGTCAAGAQGCATGTGGTGACTTGCCACTCCTGACTGAGGGCTAGGGTAGAGCCTCCCATGCTTCTCTTTTGTCA
AGTTATTCTTTGTTGTCAGTGCTGAGTGCTTCAGAAACGGAATCCTGCGAAGCCCCCAGCCTCTCCACAGCGTCCACAATGCACTCCGTCCACA
ATGCACTAGTGAGGTCTCTGGACAGCAGTTATCACTGTGCTGTGTGATTGGTGGTAACTTTCTCATTTTACATTCATTACTTGGAATTCTACTG
CAAGGAAGATCTGTCTCTCTTCCCTCTGTTGATTCAGCCTTTTACTTTGTCACCAACCACCTAGGCCTATTGTCCCCCTCCCATAGGATGCTTT
CCCTTGCTGCTCAGATTGCCCCAGTGTTGACTGGTGGGAACACCTTCCTTCCTGGGGTTCACCACATTGCTGCCTGTGCTTTGTTGTTGTTGTT
GAGAACATCCTTAACTCTTTTTGCTTTGTCTTCCTCACTTTATCGTGCTCTGAGATGTTCTGACATCATGCAGTTTCCAACGAGCTCTGTTCCC
TTCCACTGGAAAATAGCATCPACAAACAGTGCCGCATGCTCATGTGTGTGTGTGTGGCGGGACTTCATGTTCCTAGAGCATCTTAGCAGACAGC
TCGGTAGGACACTTCCAAAGTATAGACAAGCCTCTGCATCTGCATGGATCTCTGTGTCAGACACATTTGTATTTAAAGATTTATTTATTATTAT
ATGTGAGTACACTGTAGCTGTCTTCAGACACTTTTTTCTTCTCTCTTTGGCCCGAACATTGATTGATTGATTGATTGATTGATTGATTGATTAT
GTAAGTACACTGTAGCTGTCAGACACACTAGAAGAGGGCGTCACATCTCATTACAGGTGGTTGTAAGCCACCATGTGGTTGCTGGGATTTGAAC
TCAGAACCTTCAGAAGAGCATTCAGTGCTCTTAACCGCTGAGCCATCTCTCCAGCCCCACATTTGTATTTAAAACATGAGTACTAGGCATAGCA
GGTAACAGCACTGCTTGCTTTCTCAGAGAACATAGGTTCAGTTCTCAGTGTGTACATGGCAGCCCACAATTGTCTATAATTCCACTCCCAGGGA
ATCTCAAGCCTTGTTCTAGCCCCTGAGGGCAACAGGCACACATGTGGTCCACAAACATACACGCAAGCAAATGACCCATGCACGTAACAACTTC
TTAAAAGCTGTGAGTACACACGGTGCCGTACAGCACCACACGTTTGTTCTCCTCCCCTCATCCAGAACTTCTCTTTCCCATACAGCCACAGTGT
GTTTACGTCTGCAGTCCTAGACTGCATCAGGCCACACATTCTGGTAAACACCTAACAGTGTCTGGGTACAGTGGATGGTATGACATGGGCTACA
TAGGGACTTTGGAGGTCTTAGTCTCAACTATTGATCTTGCAATTACTGCCTAATTTACATCTTCAGACAACTTTAACACCCTTGAAGCATCAAG
TTTTCTTGTGTCTAAATGGGGCTGATTTTTCCTGTTTGAGTTGTGATAGTTGTGGCACAGGCAGGGGAACCTCTTATACAATGGCCTTTCCCTT
CTAACCCTCTCTGCTCTGTTTACATAACTTGTTTTGCTTCCTTTACCTGGATCTATGGAAGCAGTTCTTGACTCTTCATTTCCTACACCTAATC
CTTGGTTAATTTGTCTCTAGTGACAGTCTTCATTTTGATTTTATATCATCTTAATAGTGGTAGGCTTTGTTCTGACTGAGTCGGCTACATGGAA
GGCCTGCTACATCATGGTCTTCTGTGCTGCTTTTATATTTGTGAAAATCATTTTTTTCTCAAAACAGTGTGCTATTTACTCTTATCAGAATTCT
CACTCTTACAAATCAGCCATGGGTTCTCCCCTGCTTTTTCTTGCCAGATAAAATCACTGTCTTGTCGTTTGTATTTCACTGTTCTCCTTTTTCC
ATCAGATAGAGAACCAGTGTGTGCTGAGGGTTAGGGGGATTGACTATGATGACTGACACCTTAGTCGTCACACTTGAGCTTAGGTGCTAGCCCT
GCCTCCATTGCAGTGCGAAACTTTTGCTGTCATGGGCAGTCGTATTACACTGAAGTAGGGCTCTGAGTAGCCCCCATCCGTCTGGTACAAACTT
GCATTTTGGGTTTCAGCTTGTTAGTGTTGGTGATGGGAACTGACTGGGGGAGCGCAGGCTTGTGGGCTCCTTTACCCCAGGCTTTCTGCTCCAT
TTGACTGTAGCTGCAGTCTGTGGTGCAGCTCCAACTGTAATCTCCAGGTTCCTATTCTGGGAATTTTTCTTCCCTCTCACCTCCTAACCCGATA
CAGTGGTATAGGTGGAGAAGGGAGAGATPTACTCTGTAAAAAGAGAAGGTCGTGGTGGTCCACACCTTAGAACCTAGCACTCTGGAGGCAGTGG
TAGGAGGACCTCTGTGACTTCAAGGCCAGCCTGGTCTACTATCGAGTTCCAGGGTAGCTCCAGCTACATAGAGAGACCCTGTCTCAAAAGTTAA
AAAGAAGAGAGAGACAGAGAGAGATGGATTAGAATTGTGAACTTATCAAATATATAAAGTAGACCGGCTCGGACTGACGGAAACTGGATTCTAT
AAATCCAACCCAAACCAGGTGTTGAGAAGACCTCTGTAGAGGTACATACCGTTCTTCCCAACCAGCTCTGGCAGAACTCCAAGCTCACAGTCAG
CTGGTCCAAGGGAGCCACAGCAGGCTCTGGGGCAATCATCAGACCTGCTATAAAGCAATATGGGGCAGCCACCCGCACCAGGAAGCCTCTGCTG
CAAGAAATCTCAGTCTTGGCTAGTGGGGACGATCTCAGTGCCCAAGCCTCCAACCCATTAAATATATCTCAGGGGAGAAACTGGCAGAGGCAAA
TAGTTTTTAAACTCCCCAGTGACTCTAGTGTCCAGCCAACGTCTGAAAATGGACTGCCTGAGGGTCTATCTCACTGTGATCTCTCCTCAAAAGC
TAAAGCCTAGTTCTAGACTGCAGCAGGGAGCCTCAGAGGCTGCAGGGGTCAGAGGTAGACTGCAGAGCAGACTAGTGTCCACTGTCTTGGAAAT
TCTTAATTATGTACTGGGCGTGGCAGAACCCCACAATAGTAATGTTGTCAAGGGTTCTTTCTCTAAAAGGAATCTTTTCCATACATCAACAGTA
AGGCAGGGCATCCCCAACCTTTTATATATAGCTGCCTAATAATACATAAGGAATCTGCACCTATATCCTTCACAATCACCCTGTTGGGAATGGA
CTGTCATGTGCCCTGCTTAGATATCAGTGACAGCTACCTGCAGTCATTTGAACATCTTTAGGTGTATGTGTGCAGATGGTGAGCATTGGTCAGC
AAGTATGAGACGGTCTAAGGCAGATGAACAGAAAACTCAGAAGTCGTTTAAGAGACAATTCAGAACTAACAAAACCAAAAACCCATCCACACTT
CTGAAAATGGCGACGATAAGGAACTCTGGAAATTGATGTTCAGTAACATTGACCTCCAGACTTAGAGCAGACGCGTGCGCGGGCAGGTCCTCTA
GTCCAGACTGGTGATCTGAGCAGTCGGCAAGGGACCAAGGCAAGAAGTGGGGGTGGGGAACAGTAAGGAAGGAGCAAAATAACCGTTCATCTCA
TCAGTCACAGACAGAAAGTGTGTTAAGACTTAGAAAGGGCAGGTGACAGWGACAAGCAGGAAGAAGTAGCTTTTCATTCAGTTATACTCTTGAT
GAAATTGCTAAATTCTTACAAGATATAGTAGAATTTGCTAGAAAAATATCCGGGCCAGCAAAAGAACACTTGAGGGCTGGAGCCCAATGCCTGA
CACCATGTTGGGTGACACACAATACAACTCCAGTTGCAGGACACTGATGCCCTCTTAGGCCCCTAACGCATCTTCTACATGTGTGCATATACAC
ATACACTGACTGCACACACCATCTTTGTTTAAAGCTGACTTAGGGTGGTGTGGTAGAACAGGGGAGAGAGGCCCTTAGAGTTCATTGGCCATCC
AGTCTAGTCAAATCAATAAATTTTAGGTCCTGTCACAGACCCTGTTTTTAAAATACTAAGGTAGAGCCTAGCATAGTCCCATCCGTCTTTAATG
CCAGCACTTGGGAGGCAGAGGCAGGTGGAGCTCTGTGAGTTTGAGGCCAGCCTGAGCTACACAGAGAAACCCTGTCTCAAACAAAAGGTGGAGA
ACCACTGAGGAAAATACCCACCATCAACCACTGCCCTTCACATGACATGCATGCATGGATCAATGACAGCTGCCAAAGGCACCAGGACTCAGAA
TGTCGACATGGTCAGGCTATTTATTCCTTGTACTTGAAGTAAGAAAAACATATCTAAAATTAAAAGTAGAGTAGAGGCTTCATGTATCTTCATG
CTAATGTCTGAGTGAGCTGTGTGTTGTTCACATGCCTTTCAAACATPGAGTGATGGCTAGCAAATGACCACAGGAAGAAAGCACAGGGTTTTCT
TTGCTTTTTATCTTAGGTTTTGTTTTGTTTTAAGACAAGGTTCCTCTGTATAGCCCTGGCCATCCTGGAACTCACTCTCTAAACCAGCTGTCCT
CAAACTCACAGAGATGCCTCCGCCTCTGCCTCCACCTCCACCTCCTGAACACTGGGATTAAAGGTGTGTGACACCACTCCCCAGCAAAATCACA
GGTTTTTAAAACTATTTCATTGTGTCTCAGAAAATAGCACTATATTCTTTTCAGGTGTTTTAGAGGTAGATAGCTCCTTTTTCTAGAAAGTATC
TCCAGGTCTTCAAGTGGAGTGAATCATAATAGAAAAAAAAAAAAAAAGAAAGCAAAACAAAACAAACAAAAATCCAAGCCACTAATGAAGTGTT
CATTTTAGGATTTCGTACAGGATAAAAGCTCTGGTGCAGTGCTGCATTGACAGCCTCCTGCAGCTTGTTAAAATCAGAGTACTGGACAGACAAA
ACAAAAGCCAGTGTGGTGGTACTCCACACATTTCGGCCCAGCGCTCGGAAGCAGAGGCTGCTGGATCACAAATTTGACCCTAACCTGGTTTACA
GAGGGCGTTCCAGGACAGCAAGGACTATACAGAGAAACCCTGTCTCAAAAAACCAAAAATAAGTAAATAAATAAGGATGTTGACTGATAGATTC
AGAAAGGAAAAATCCTATAAATTGGTGTAAATTGCCACTGTTTTCTCTGCTTTCAGATTAGTCATTAAGTAAATACTTATATAGGGGTCCTGAG
CTGTGTGCTAACTGTTGTGGCATACAAAGGCAGTGTGGAGCTCTGGGCTGGCCTGCTGGGACTCTTCCTTCTACTTGACAACTGTGTGGGACAA
AAGTGCATTCTTACAGGGATCCTAAAGTGTTCATTGGAGGGCACCTCCCTACTAATGTGAGGAATAACCCTGTATGTTGTTCCCTGAGTTCATC
TCCACACCCACACGTAATGCACTGCTTGCCTATCAACAGATGAATGAATGAAATGCCAGCTCTGTTCAAATGCTTTAATGTAGCGCCTGTCTTC
TTTTTTCTCTTTTACAATTAAAAAAAAAAATCAGTTGTATGCTTATGCATGCTTTACCTGCGTGTATGTCTGTCTACCATATGTATGACTGGTG
CCTGAGGAGGGCAGAAGAGGGCATCCTATCCTCTGGAACTGGAGTGATAGGTAGTTGTGAGCCACTTGTGGCTGCTAGGAACTGAACCCAGGCC
CTCTCTACAAGAGCAACAAGTGCTAACAGCCAAGCCATCTGTCCAGCCCCTTTGTTTTCTTCAGAGACAAGGTCTCCTGTCACTAAGGCTGGCC
TTGAAATACTGGTCCTCTTGCCTCTCTCTGCCTCTTGAQTTGAGTGATAATCCCCCAAAGTGCCCGTGCCACTATGCCCACTCTAAACTCTTAG
TTATTTACTTTGTGTGGGTCTGGGACTCATACCTCGTGCTTCAAGAGTGCCGAGTAAGCACTCTACTCTGAGTCCTATCACCAAGCTCCTCAGT
ACTTAAAACTCCCTGTTGTTGTTGCTGCTATGTGTGTTTGCTTGACAACACGTCTCACCAGGTAGCTCTCAACCTCCCAGCTCTTTATGAAGCC
CTAGCTGCTCTCAAACAGGCTGGGATCCTCTTGCCTCTGCTTGCCAGACGCCACTACACTTAGCTTTGGAATGCTTCGCTTCTCCTAGCAGTTT
CAGGCTAGCCAGGATCATCTCACCAACCGAGGAGAGACCACAGACAGAGTTGTTAGGCGGCAGAATCACACTGGCTAATGTTGAGGTGTGCTCA
AGTCCTCTGCTGTTGTAAAAGGAAGACCAGTTAAGATCATAAAAGCCATTCCAGTAGTCAGGACCCGCGCCTGCCCTGGGCTAGGGTCTGGCTC
GCAGTGCACCCTTCCCCTGAGTTAGAGCAGGAAGGGAGGCTGTGTCGGGGGAGTCTGAGCACCATGCCTGACAGGAATGATGTCCTTGTGGTCA
GTGGTTAATGTACCTCGCTGCTTTACAAGACTCCCGGCTTGCCTTCTTGGGGCATAATAACATCCACTCTCCTGGGAACCCTTGCCCAGTGTCA
TTGACTGGTCCCACCCTGGGTTCTGCTAGCCACATCACCAGTCTTTCCTAGTAGTGATGCCATTACAACACTGCCGTTCAGAGCCCATCACCCA
GTCTTTGGGGACGAGTGCAGAGCTATCAGACACCAGCTACTATAATTAGAAAAGGGAAAGCAGGCGCTCACCGAGCTCACATCTTAGGCTGGGG
CATTTACAGCACTTTTTTTAAAAAGTTGCAGCCTCTTCCTCACTTTTGCTTCCTGAAATACAAGCTGTACACTTTTGGGGTTCTCACATTTCAG
TTTTTGTTACTGCTGTATGCTCCCCACCCCCATTTTAATCGGAGAACAAATTGCTGTCAGCACATCGAGTTCATGTGCCAGCCACATTCAGAAC
AGGGTGTTCTGTGCTCTTCAAAACAAAATTGCTCTAGGGCAGTAACAGAACAATAACTCAGAGCTCAGTTTAAGTGATGTCTGCAGTGACCAGA
CTCACTTGATGTGCTTACACTCTGGATTGCACAGCTGAACTTGGCTAGTTTGGTTTGCTTTGACTTTGTGGGGTTTGTTGGAGTACCCCCCTTA
CAGGTTACATAGACTTTTAAGTCAAGAGAAAATATTGCCAGTTTTAAACTTGTTTGTTTCTCTTTACCAAAAAAAAAAAAAAAAAAAATCCCTA
AGAAAAGCCTCCTGTGTGCACTTGTGTTCACTTGGACACCATTGTAGATCATTTGCTTCCTGTAAATCATGCPGCATCAGATTAGCAGCAGACC
TCCTGAGCCCTAGAGGCTGAGCTCCCGAGTGAGTCATGGTGGCTTGTATTAAATATTTATGATATGAGTATTTGTGCTATGGAATACAAAGTCA
AAAGCACCCACAGGTCTTCCAGAGTCAGAGCAGGGGAAGTGGCATTACTACCAACCCGGCGCCTCTGCATGGATCCGCTCAGACCAAGTCCTTA
CTGTGCAGAGGCCTGCCAGGGCCCTGGTGCTGCCAGCTCTCGTCAGCAACCTTCCACGACTTGTCACACCAGTGTTCAGAAGCCAAACAATCCA
GCCTGTGGTGACCTTTCTCCATGTACATGGAGACAATCATGGGCTCTGTTTACCAGCTCTGACCCTTCATTTTTCCTGACTTAACATTCCATCA
TGTCCTGAAGTGATTGGTGCATGCATGTATGTGTGAGGAATCGGGAATGTCACCTAAGATCTCTAATTTGTATTATTATGTATGTAAGTCTCCA
CGTACAGTGAGAAGAAATGATTCCCCTCTTCTACCACTGACGCCCAGGTAATAAGATTGGGACATCAGGTGCACGCAACCTTGCGCTAGCTGCT
GATTTTCCAGACACAATTGGACAACTTAAAAAATCCACGTAACTCCATGTTCTAGGGCTCTGATGCCCTCTTCTGACCTCTGTGGGCACCTGTA
CATGCATAGCATATAGTCAAACACACACGTATGTAAATTAATAAAACCTTTTTTTTTCCTTTTTAGATTTTTATGTGTATATCTTTTTTTGTCT
GAATGTATCCATGCAGTGCCCATGGAACCCAGAAGACGGCATTAGACCCCAGGAGCTGGAGGCACAGACAGTTATTACAGTTACTGGCTTCCAT
ACAGATGCTAGGAATGGAACCCCAGCTCTGTGGCACAAGCAGCAGGTTCTCTTCCCCACTGTCTCCAGCCCCAGCAATTGCCTTCCTGCTCTCC
CTTTGTCACATCCTGACTTTCTGGTTACTTTTTGCTTTCTTCTGACATTCATTCATCTTTCCTGCACACAGCGATGCTTAGCACACCCAAAGAG
AATGTCTGAAATTTTTTACAGACTCTGGGAATCTCACTTAGGTCTTTGGTTTTGTTTTGTTTTTTTAAGATTTATTTATTATATGCAGCTGTTT
TCAGACACACCAGAAGAGGGTATGGGATCCCATTACAGATGGTTGTGAACCACCCTGTGGTTGCTGGCAATTGAACTCAGGACCTCTGGAAGAG
CAGTCAGTCAGTCTGCTGAGCCCTCTCTCCAGCCCCCTGTTGTGGATAGCCCTGGGGCTAATTATATTTGTTAATTCTGTTCTCCTGTGAGTAG
TTTAGAACAAGGAATGAGTCAGCACTCACGTGATTTCCTCTAAACCTGCTTCCCACCTTAATTTGTAAAATAAAGGAGAGCTGATGATTGGGCA
GATAAAGGQAAGGTGGAGAGGAAGGTGAGAAGAGAAGAAGGACAAAATGCCAGAGAGAGAGGATGCAGAGGAGGAGAAAQAAGAAAAGCGGACT
AGAAGCACATGGTCTGCAGAAACCGCAAGTTCTTTTTTTTTTTCCACTTTTTATTAGGTATTTAGCTCATTTACGTTTCCAATGCTATACCAAA
AGTCCCCCATACGCACCCACCCGAAACCGCAAGTTCTAAGGGATCTCTTAGATGGAGAAGATGGCAGTGTAGCGGTAGATCTGCCCAAACTAGG
CACACATCATGTATTCATATTAATTGAGTTGTGTTTTCATTGCCGGAGCATATTTGGGTTAGACAAATTTACCGCAACAGCCCCCCTCCCCACA
CACTTAAGATTTTTAATTACACTTTTATATATTATGTAGATAAGCTTGTACATAGAGGTAAGACATTCCACTCTGCAAATCAGCAAACTGATTG
GGGATCCTTTGCAGAACTCTGGATTTCCTCTTTTGAAAACACATTCACATTTGATCAACCAAAAGCCACAACAAATGTAGTTATAATTTCCTCC
GTGCTGAAGGCACTCCATTGTAGGGAGGAAGQTGATCGTGTGACAGTAGACCTCAQTTCTCTTCCCTCGAAAGTCTGTTCAGTTGGCAGTGCCC
AGACTGATAAATCTCAGTACCAAAGAGCTGCTTCCGTGGCTCGCCAGGAGTTCCTGCACTCACGGACAGCACCATCCATTAGTCTAAATTCAGT
TATCTGCACAAATTTAAAAACAGTAGTTACATGAACTTATAGTTTTCTTTGATTGGTTCTTCCATGTATATTTATTTATTGTGGTTGTTGGGAC
GGACCTCAGCCTCTGTACCTGTTAGGCAGGTCCCTAGCACCAAGCTACACTTCTTTCCCTGCCTCTCCCTTTACCTCTCATCCTTAGCTAGCCT
ATGGCTGTCTGAAGACCTGGGCCTACAGGCCCAAGTCAGGCTCAGCTTAGACACCTAACCTAAGAACCAGGAGACACCACCTCTTCTCCACAGA
GAACCCCATAAGCCTCAGGGTGTGCCCAAGCCTCTGATGCTCACACTTTACAATATGGTTAATTCTTTGTTTAAACTACAAGATGCTGTTGACT
CCATCTGCTGCCACCCAGCTGGGTGCCTCTGTGCTGTATGCAGCCTCCCTTGGTTTTGTTTTTTGTTTTTCCTGTTGTATGTGCACTAAAAGTG
GGGGGGGGGGGGCTGTAAGTTAGCTGTTTTATCTAGAACATTCCAAAAGTTAATATAATATATAGAAGTCATCATATAAATGAGTATGGCTATA
AAACAAGAGACAGTAGGCGACATCTGAGAGCTAGGCTCCTGTAGCCTTTGTCTTGTAGGAACTGACTTACGATTTGACTTGACAGCAGTAAGAG
CAGCCAAGATGGTGTCTTTCCTAGCTTTACTCCGACTCTGAGGTCCTGATGTATAGCTTACCCCTGTTTATCTTCCAAATTTTCTCTCATTTCT
CAGGCCTAGACTAATGGTGATCCGCTCAGATGATTAATTAATTAATGTGGAATTAGCTTTGAAGCTAGTTTCCAGGACGAAACAGTCATAGACT
TTGAGGGCTTTTAACAGACTGTTGAGATCAGATAGTAAAAGTAGAGTAAGTTGTAATTGGATTGCTTGGAAGATGATACTATGGAACTAATGTT
TAAGAGAGGGTGGTTTCTCAACACTAAGGCAAATTACTGGCCCAAAATAAGTTAAGCGGCCTTGTCAAGAAATATTTTAGGAAATTACTATATT
TGAGGTAGAAACTACTCTTCTTGTCAAAGAGAGATCATGGTGCCTATCGAACTATCTCACAAGCCGTCCATTTTTCTTACTGAGTAAAAGGCCT
AATTGAAAAGGCACATAGCATGAGTATTACAAGTAGTTGAGCTGGGGGGTCACTTAGTGCTTGAGTGATGGCCPTATTGCACAAGTCCCTCGAG
TCTCCATGACCACACAAGAGAACTGGATACATGCCTATAGTACTAATGCGCTTGGAATACACGGCCAAGAGCAAGAAGAGTTTCAGGCCAGCCT
GACCACGTCACCTTCATCTAACATGTATGCAGTATTGGAGAGCTAGCATCGAGACCTTCATCTAACGTGTACAGTATTCGAGAGCTAGCATGGA
GTCCTAGGCTTTATTTCTTGCTTATATACTTTTTCCCAAGCATCTCAAACCAAAATACTGTCACAGATGGTTAAGCCATTGTATGTATTCAATA
CTAGATATATGGCATTCTCCTAATGGTGTGTTTAAATGAGCTAGTGTATCTTGACTCTGTGTCACAGATAACAAAATAAGTATACATTTAACTT
AAAATGGAAAAACTTTTGTTCTTAAACTTTCAGACTAGGTGGGCAAAGCTTCCAGAAGACACTGTGTGTCTTATGAACTGCAGGCGAAGTAGTG
TTACTGCACTGTGGGCCACTCACTTGGCAGGCAGTGCTGAGGGGTGGCTTGTGACTGTCCTCTGTCCTCACCCTCAGTCCACCTGGAAZGCTGC
TGTGGTGGAAGGACATTTGATTGCCCAGATGTGGTGTTCGTTGTTGGGGAAGGAGAAGACCACGAACATTCCGATTGGCATCGACAAAGCCCTG
GTCAACATGCAGAGACAAGAACAGTGTATTCTATATCTTGGACCACGGTAAGACGTGAGCGCCCCTTTCAGTGCAGTGCAGCCCGGCCGCCTGA
AGACCAAGGACTGCTTGCTCACTCTCATTCTCACTCTCWCAGTCCCTTGTTGGCGCAGGGAAGCCATTCCATTGACTTGTGTGATGGTTACTCT
GAAACTTGGGGCCTCACACCACCATAGAAGTTTGCGAGTGAGTTGGCTCAGCTGGAGTATATCTAAGAATACGACTATCAGTGTAGCCTTGATT
TTGATTTGTTTAGCTGCCAGGATGCCACATCAGCAAAGATTGACTTGATCCTGTGGCTCTCCTGCTCTGCTTTTCTCTGAATTCAGCGAGGGCT
TTCGGAACTGCAGTAGTGCATGATTTACTACACTTGAATCCATGCCACGTTCTTCTATGGAGTCCCTGAAGGAAGAAAGAGCCAGCTTGTTCTT
CCTGGACCTAAAGGCAGCGGAGATGACTCATTTTTGCCTAACTTTTTCCCGTCATAAGGATAGCCATTAGCACTGCCAAGCTTAGAACCATCCG
GCATCACAGTCTCCTGTGAAAACATGCTTCCTATTATTTTTAGATCTAAAATTATACCATAAATTCATCTCTTCTATCTTGAGAAATGGGCCCC
TGAGGATGCAGGAGTGCTGCCTGTGTGCAATCGCATCTGCTTGTTGCCACTGCTAAGACTGAGACTCTGTGGGCTGCCCTCACCAAACGGAACA
CAGTGCTTTTGTCCTGTCTCTTGCCTTCGACGGCTTTTGCTATAATTCACTCTCCAATAAAGCTATTTCATTTTTATTGAAAGAGCTTTCCCCC
CTTACAGAGTAGGTTCTTCATAACCTACAGTCACTAATCTGTTTTTTGTTTGTTTATGAGATGTATTCTTTTATGTTGTAGCCCTGACTGCCCT
AAAACTCAGTATGTAGACCAAGCTGGCCTTGAACTCACAGAGATCCACCTCCTTCTGGCTCCCAAGTGCTGGGATTAAAGTTCTCTGATTATGT
TTAATCTACCATTCCAAAATTATAGTAGCTCTACACTCAGGTATCTCACGTATGGTCTTACAGTTCACTTCTTTAAAGATGGCTCTGTAAAGTC
TGAGCATTCAGAAACACATAGCCCAGATCTAAGTTTTAACCTTGCGGTAGATGCTPTGGAGAGCAGACTTACCAAACTTAACAGCAGAGTGAAT
CAAACTGCCTCTGTTTATTAGTGCAGCTAGTTCTCTCCACGTGTACAGTTCCTGGCTGAACGACTCTCTGCAGCCTACCAGTGGTGGCACCTGG
GCAAGTCACCTAACTCCCCAAGCCTTGCAAGGTTGCAGAGGCCCACATGGAGTAATGAGGCAGATCACTTAGCACACCGCCCCACCCTGCCCAT
TAGCTGTTACTGTCTCACCGATGCCTTTCATTCATTTTCCCCTTAGACCACGAGAAAATAGAAATCTCTGCAACAAATGGTATTTTTCAAATCT
TTTCAGGCATAAAAAGAACGTCAAGTTCTCCTACGTTGTATATAAAGGGGTTCCTCCCCTCATTGGGGGTGGGGCGTGTGAGTCCATATGTTAG
TACATCTCCAGGGCGTTTGCTGGTACATGGAGTTCTCCCAATACATTCTATGTAATGCTCTCCATACTGTCTCCATGTGTGTGTGTCACACTGC
CCATGTACGTAGAGCATCCCTAAGCAGATTGGCTTGTCCTACTCAGAGTCCACCGAGTCTGGGTTGCTTATAAAATGCAGATGGAATTCGTACT
CAGTGTCTTCTGCATTTCCCTTCTACCCTACCCCAACCTCACCCCCACCTTGACATCTGTAGCCTCCPTGGCAGGACGTTTTCATAAGAAACTA
ATAAAGCATTCAAGTTTCTCTCTCTCTGGAACACTATCTTTTCTTGGTAGTGTTCTTTACAATATTGTGGCAAGGGTGTACCCTATTTGAGACA
TTGCACCCTCAGATTTTTTTTCTCAATTTTTAAAAATTTGCTGTTCTCCTCAAATAGATAATTAAATATTGTGACTGATTTCTATCTAGGCTAA
GTATAGATGACTACTTTTTCCTTCCTTTTTCTTTATACCTGTTTTGTAACAGTCCCAGACAAACCATAGGCTGTTGAGAGTGGTCAGTGTGTGT
CAGCCTTAGTTATCTTAGGACAGGAAGGTCTGAATGTGGTTCTGCCTCTATATCACCTCTCAGAAAGCACATGTCTCTGCCCTCCGCTCTCTCA
CCTGTAAGAAGTAGCTGGCGAGCGCATTCACAATGCTGTGTTCTGCAGGGTTGGCTGTCCCTCCACGCCCTTAGTGTATCTTCCCTCCAAGGCA
GCTGCCGTCCAGAGGCCTTGACTCTGGACTTCCATTTCTGATGGTTGTGAGCCACCATGTAGTTGCTGGAAATTGAACTCAGGACCTCTGAAAG
AGGTCAGTGCTCTTAACAACTGGCCCATCTCTCCAGCCTGTAAGTTTCTTTTACACAGGTTTTCATGTCCTCAGTACTATGGATGTTTGTGGTG
GATCCCAGTTGTGGCAGAAGAGGGTTGTCCTTGTCCCATAAGGTGTGCAGTAATATCTCTCACACCTACCTGCCATCCAATAGCAACTCTACGC
ATTGCTTAATGTTCCTCTGACAAAGAAGGGTTAAACATCAATCCACGTAAGTGACATGGTTCCAAAGTGATGCCAGGTCATCTGTCATATTCTT
TCCTCCCTGGTAAAGACTGCCAATCATAGCATGCTCTCAGCAACAGACTTCAGAAGTACCCATCTGTATGCCTTCTTTGCAGCGAAGAGAGGGA
CAGACCTTACCACAGGCAACACACACACATTCATACTCCAGGGAAAAAACCCTGGCTACGTTTCCAACATTGCCACTGGCCATTGCTCCCTTTT
CGAGTGATGCCCTCTGCACGCTGTTAAGGCTGCCACCCAGTTCTTCCCCAGCTCTGTGTGCCTCTGCTCCAGCTCTGCCTTTTCTTTTCTTTTT
TTTTTTTTTTTTTTTTTTTTTCGAGACCGGGTTTCTCWGTATAGCCCTGGCTGTCCTGGAACTCACAACTCACTTTGTAGACCAGGCTGGCCTT
GAACTCACAGAGATCCATCCATCTGCCTCTGTCCCCCAAGTGCTGGGATCGAAGATGTGTGCCACCACCTTGTATTTGTGCCTTTTAAAGTCTT
TAATGCCACGAGACTTTAGGATCATGAGAGCTGTTGTCTCTGCWAATGTGTTGCCCCCTGCAGTTTTTCTGATTGGAGGTAGTACTCTTGGAAG
TTGACCCTTCCTTAAAGTGAAAAGCTTGTTTTGAGTACACATGGACATGTGGGAAAGAGTTCCTGTGCGGGAGGGAGTGGTGAGGGAGCTGGGG
TAAAATGAGAAATTCTATATACAAGTACGAAACTGTAACAGAACAGTAAAAACAATTGTAATAAAATAGCAAAAATGTTAAAAGTATTCAAACA
GAATTATAGTGATTTTTACTACTATAATAATTATAGTAATAGACTCTTCTGTCCTTTCWGCTCATAAAGCTTAAAGGAAAAAAGTCCTTGTAAC
CAAACAGAAAAGAATAAAGTCTAAAACCAGGCATCGATATTGAACAGCATGCTAAGCACACTGTCCTAGGGAGTACAGGAACTGTGCGCCTGCG
CTAAGAGAGCATGCGCTAAGAGAACACGATGCAGCAGGTCTCAGGAAACCACAGCAGGGATTCCACACAGAAAACCAAAGAAGTATACCAGTCC
TCCTGAGTCTTGTTTTTTCCTCTCCGTTTCAGCTATGGTTTTGGAGAAGCCGGGAAGCCTAAGTTTGCCATTGACCCCAATCCTCAGCTTATGT
ACGAGGTCACCCTTAAGAGCTTCGAGAAGGTGAGCAAACCCAACTTCGCTAAAGCTGTGGTGTGGGGTCTTCTCATCCTGTGCCTGTAACTGTG
CCGTACATCACTTCGCTAAATAGTGTTTGTTTCTGTGTTTGCTTTTCTTCATCCATGACTCTTGTCCCCCACACTTCTGCTCCGAGCGTGGGAA
GCTGACACAACACAGTTCATACACGAGAGGAGCTGGTGTTCTTATTTTTTACTGTGTGTTAGCCTCTTTTTTTCTTTTAACTTTTGGGTAGAAT
CCAAGACTTGTTCCTGTATTTCTTCCTAGGCTTCTTCTTTTTGTACTTTTTTTTCCTGAGTTGAGGACAAAAAAGTTCACGTACTGCCAAGCAG
CTCCTTAGGGGAGTTGGGGTGCAGCCTTTCAGTGCTGAAGGAATTGCACACTCCACAGGAGTTACAAAGAGGCCTCCGCTGGGGCTCCAGGTTA
CAATCCCAGTGTCTCAGCCTCGGGACCAGCAGCTGCAACCAAGGAGAAGAAACAAACGPACCCAGGCTGTTTCTCATACGTGTCCAAGAACAGG
ACAGCGAACTTGCCGTCGCCTTCAGGCTGGTGTACCTCCTCGCACAGCCTCCAACTCTGGGCTGTCTTTGCAGGCTCCCACCTGTACTCCACGG
TGTCCCCATAAGCTGAGTAAGCTGTCATCCTCAGCTGTGGCTCCCCTCAGAGACCGCCTTTCTTATTACGATTTTATTCCTCTGAAATGACCCC
ATGCTCTGTCCACCCTCGACTATCGGGGCCTCGAGTTTCCCTCCCCTTTCCATAGTGGAACCTCTACCCTTCTCCGTGCTTTGTGACAACATGG
CATGCCTGCCATCTAATCCTGTCACTCGGGAGGATCAGAAGTCAGTCTCAGCTACGTAGTAAATGGAGGCTTGCCTGAGATACATCCAGTGCTG
TCTGGAAAAAAGAAAAGAAAAAGTTCCCCAGGTCCACACCTGCAAACTGATCCTGTTAGAGGCCATACTGCATCTGGTTCTGTGAATTCGGAGA
CACCTGCCTGAACTGGGATTTTAGAAGTGCTCCATGCACGTGCAGCTTGGCCTGGACCATAAGTAGCAATCTCACTTCCAAGCTGGAAAGCAAG
CCTGACAGAAACAGCCTGCTAACGTGACAGCAGCCCATAGTGAGGGAGCTTTGGGGCTCCAGGTTACGATCCCAGTGTCTCTAAGGACGGAGGC
TTGCTTTGTCACCCTCAAAATCCAGTCTGTGAGAGTGCTCCGTGCTTGTGTTCCTTGGACAACACAGGTCACACCACAGCTTGCTATGGCATGT
GCTCTTAGTGGAACACCGGTGCATTGGCCTGCTGGTATCCCAGCAGCGGCCCACAGTTGCTGGGGTCTCATAGAGATCGCTGCTGTCCCCCAGG
CATGAACTGCTGCGTTTCCTCCTCTCCTCACCTAACTGACGCCACAAATCTCTACACAGGCCAAAGAATCTTGGGAGATGGACACCAAAGAAAA
GCTGACGCAGGCTGCCATCGTGAAAGACAAGGGAACTGTGTACTTCAAGGTGTGTGAGTCTCTAAGTCCTTTCCATGTGGGGATACCTACCCAG
TATCCATCAGCACCCTGTGCTCACACGCACCCTCAGTTCTCACAGGCCTGCTGTGCTCTGTAGGAGGATTAGTGAGCAGAGTGAGTTCGTGTGC
ACQACGTAATGCAGAGGCCTCTTCTGTGTGCTGTGAGCAGGTGGACCTTCCTCAGGAGCACCCGAGGTGGACCAGACCTGGCTCCCTCCAGTGT
GTGTGTCCCAGGTCAGAGTTCACAGGGGCGGCTCCAGCCCGCTCCCCCCWGACACGTGAGACAGGAAAGGCCWTGCAGATCCCCCAGTTCACTT
TGGATGATCTCTCTCACGTTGCCGTQCTGTGGCTGCTTTTATGTAACTACAATGAAGGCAAGTCTCCTCCTGATTGTTTTAATTATAATTTTCA
ATTGAAATTTTTTTTAGGCTGGAGAGATGGCTCAGCAGTTAAGAGCATTGGCTGCTTTTCCAAAGGTTCTGAGTTAAATTCCCAGCACCTACAT
GGTGGCTCACAGCCATCTGTACAGGGATCCCATGCTCTCTTCTGACATGCACGTGCGCATGTGCAQAGTACTCATAAATAAAATAAGTAAATAA
AAAATAAAAACAAATCTTAAAAATGTTTTAAAACATTTTCGCTCTTTAAATTAAAACAGCAGCTTTCACAGCAAATATCCTCAAAATAAATGTT
TAATATTTAAGGTTTTATCAAGAAATATATCATTACTTATCAGTACCACTATTAACTGAGCAACAAGATAAAGATGCCATTAATGATACTGGCT
GAGGGGCTGGCGAGATGGTTCATCGGGTAAGAGCACTGACTGCTCTTCCACAGGTCCTGAGTTCAAATCCCAGCAACTACATGGTGGTTCACAA
CCACCCATAATGAGATCTGATGCCCTCTTCTQGTGCGTCTGAAGTCAGCTACACTGTACTTACATATAATAATAAATAAATCTAAAAAAAAAAA
AAAATGATACTGGCTGGGTACCAAAAAGGCCCTGCAGACCCGCATGGATGACCAGGCTCCGCCCTCCCAAGAACCTGATGAGGAGAAAACACTG
AGCCTTGTGGCCACAGTGATAAGATGTCACTTACAGCTGCCCCAGGGAATTACflCCTCTCCAGAGCCCTGTCTCCTCAGGACCCAGGAACCAG
TGCTTTATCCCCCAGGGCTGACACCCACACTCATTAAGTGAAATTTCGGAGTGCTGTGCCCATTGCTTGCCAGCCCACTTCCAAATTCCTGCTG
TCTCCACATCAAGTGAAGGGCTTCTCGATGAGTGTTCACAGCGGCCACTCTTCTGCTCGCCCAAGTGATCCTCTGCAAGTCCCTGACTCCTCTC
TCTGCCTTGTAGTGTGGAGACCCAAGCCACAAGCCAGCTTTGTTCGCACACCTTTAAGCACAGGGCTCTGGAGGCAAGTGTATGTATGTGACTT
TGAGGCTAGCCTGGCCTACAGAGCAAATTCCAAACAACTAGGGCAACACAGTGAAGCCCTGCCTCAAAAAAAAAAAAAAAAGGACTTGGAGGGA
ACCCAAGTTTTCTTTGCTTTTTCTTTCTTTTACCAGCAGACTTTAAACACAATTATTGGGGTTTTTGTTTGTTGTTTGGGTTTTTTTTTTTTTT
TAAGATTTTATTTATATGTAAGTACACTGTAGCTATCTTCAGACACTCCAGAAGAGGGAGTCAGATCTCATTACGGATGGTTGTGAGCCACCAT
GTGGTTGCTCGCATTTCAACTTCAGACCTTTTGAACTCTGGACCTTCGGAAGAGCAGTCGGGTGCTCTAATCCACTGAGCCATCTCACCAGCCC
CCTGGGTTTTGTTTTCTTTTGTTTTGTTTTGTTTTTTATAGCACTATAAAGCAGAGACCTGAGGCTACTCTTCATGAGCCTCTGTACTGAGAAA
TGCTGTCCATCTACTACTCACTTACATTAGGGGGTTGGACAAGAACACCATGTGGGACAACTGCAGAACTATGGGGCCATAGTCAAACCTCAAA
AGAAACACTGGCAGAGACAGTCTGTATTTGTAACAAAGTTATAAACTAACGTATGACTCATGGGCAGCCAAGCAGATGGCGATAGGAGCCCTGA
GCAGGGATCTGCAAATGGAGCTGTCCCTTCCATGTCATTGAGGCCAATCACCTGTGTTGCAGGGAGCCTTTCAGGCGTGATAAGTAPASTTCAA
CATGTTTCAAATCTTCCTAAAATGACCAATCTGGATAAAATCTGGATCACTAGTCACACATACGGAACACATTATTTTTAACATGTAATCTGAT
AGACAGGATCTTTCAAAACATCAGTCTGTTGGGGGGAAAAAAATTAATGAAAGGTTCAAAATCACTGACCTAAGACTTACCCCAGAATGTCTCT
GAACATTTTGTCTCACCAAGCACTTCCTCGACGCTCCTTCCTCATTCTACGTGAGGAGCAAGGGCACCGCCCACCGCCGCCCCACCCCCCCACC
CCCCCAGTCAGGCTGTCTGCTTCACACCCTGGAGGACTGCCTTCTGTCTGCTTGTCCCTTCTCCCTTCAAAGTTTAAGGAGCCTTTCTTTTCGC
TGTGCTGCTCTGGCCAGCGTCATCTTCCCACAGTATCTGACGTGATGTGTGCCATGTGCAGAGTGTGGGTGACTGTAGGGATGGGCTGGACTAC
AGTTCTATGTCCCTTAATATCTGCATGATCTATTCACCTGCCAGGCCAGTTTCAGCTGTGGGCAGAGAGGGACTGAATGTGGTATTTAGACTGA
TCTTCAAACTTCCTTTCAACCTCTGAACACTCATGCAGAGACACAGAAAGCACTGTCCTCATCCGGTTCTGAAAGTTACTGTCAGGCACTGTAG
CACTGTCACTGCACAAAGGAAGGCTAAGGACGCTCTGCTGTGTGCGGGCATCTTGCGCCCCCTAGTGCTCCTTGGGAAGGAAGTGTGGCCGCGG
CTGGCTGATTGTGCTTGAGAGCTGGAGTGTTGTAGGAAGAGCTCTACATCGTCATGGCTCCCTCTCCCGAGTGCAGTCAGGTCTCCCATCCCTT
GTACAGTCGTACACGAAGGGTCCTCTGTATTCAAGCTTGTAATPACTGTGCAGAAAAGAACTGTCACCTATATTCCAGGTTTACTCGTGTTGTT
ACAAGCTGCCATTCCGTGGTGCAGCTGTACCTACAGTAGTCAGCACGCTGTCTCCACCCCACAGTTTGTTGTTTAGGCCATGTGCTATGTACAA
CATTCAGTGTTTACAACTCTCAGAACTCAGTAGTAACCACGACCATATCTAGAGTCTGGTTACTCTGAAACAGCGCCTCACAGGGGTCACATAT
CAGATATCAGGTGTCTACGTTCTGATTCATAACAGTAGCAAAATTACAGTTATGAAGTAGCAACAGAAAACGTTTCATGGTPGCGAGTTTTATG
GTTATGAGAAAGGGGGTCATAGCGTTACGAAGGCTGAGAAGCACTGCCTGGGAGCTGAGGCAGGAAGACTGAGTTTGGCACTAGACTTGTCAAT
TTAGCAAGGCCTTTTCTGTCTGAAATAAAAACATCAGCCTAGAAATAGAGCAGGGATGCTGCTTGCTTACTATCCATGAGGCCTTGCTACGGCG
GAGGAGGCTGCTAGACCAGACAAGAGCAGGACAGTCTCTCACACGGTAAGCTTGCTCTTTAGCGTTATGTACACTGTGTGCTATCAATCCCCGT
GGTCAGCACAGGGCATTCTGGCCCACCCACTCTTAACAATCACATGGTGCCGGGGTAGAGCCCCGGCACTGCAGCTCTGCCGTGCTGCCTCTGC
TTTCACTGCTACTGAGAGCCGAGCGTTTGCACAATCTGCTTTAGACACGCCTGCTGTGTCGGGACTCCCACTTACCATATTGCAGTATTTACTA
TCAGTGCTGAATGCCAACAGAAAAGAGGAAAGTCACCTCCTCTTCCATAAGCCAAATGGCTCACCATCCTTTTGAAAGACTGAGGAATGTTGGT
TCCAAACAGTGTAACCTCCTGCTGCCAGGCTGAAAGAACACTGAAGCAGCCATGCGGTGCCATTACCCTGCTCCAGGCCACCTCACAGGCCAGC
AGAGCTTCCGCAGAACTAGGGAGGGACAGGAGATACAGTGGCGCTGACATCCACAACTATTCCGTGTGAGCACTCGGCTAGGGCACGCCACGCT
CACACTTCACTGCATCCTTTCCAGATGGATACAAGTTGTTGGGGGCTTTTTTCTCCTAGGGAGGCAAGTACACGCAGGCCGTGATTCAGTACAG
CAACATAGTCTCCTGGCTGGAGATGGAATACGGCCTGTCAGAGAAGGAGTCCAAAGCCTCAGAGTCCTTCCTCCTCGCAGCCTTCCTGAACCTG
GCCATGTGCTACCTGAAGCTCCGAGACTACAACAAAGCCGTGGAGTGCTGCGACAAGGTGGGTGAGCCGTACTTCACTCTCCGTTTATCTGTGT
CGCACCTCTGCCCCCTTCAGGCTGTTCCTCTATGGTCACCATGACCTGGGACGCCATAAAGGATGGGGCTTTTTGTGCACTACTTTTATCCTGC
ATTTTACATAAAGCCATTAACAGAATTGATAAGAQTCTTCATCTGTGGTCTTCCTTACCCTGGTCCTTTGTGGCAGAGGAGACAAAGACCCTTG
AGGTTCCCTCTTTATTCTTTTGTTWGCAAGGCTGGGGATTGAACCCAGGGCACTGTCCATCCTAGGCAAACACTTTACCACTGAGCCATACAGC
CTACCCCCAGTGACACACTTCCTCCAACAACCCACACTTCCTAATTTCTAATCCTTCTAATCCTTTGAAACAATTCCATTCCCTGGTGATGATG
TATTCAAATATATAAGCCTGGGGGGGGGGGGGGGACACATTCTTATTCAGAGCATTTACCACCATGGTTAATGCCTATGGTCTCAGCATGTGAG
AGGCAAAACACAAGAATTGAGAGTTCAAGGCTCCCCTGCQCTACATGAGTTCTTGTCTCAAAAAATAACTATTATCAAGATACCTCAGCTGGTA
AAGCCACTTCCTGTCAAGCCTGATGACAGGAGTTTCATTCCTAGGACCTTGTGGGAAAGAAAGAACAACTTTGTCATATGTCCTCTGGCCTCTT
CCTCATTCATAGTATTACATGTGCATACACAAAGGTAATAATAATATATTATTTTAGAGATTTATTGATGTGTATGAGGACTCTGTACCGGCCC
ACCAGAAGAGGGCATCAGTCCTCATTACAGATGGTTATGAGCCATCATGTGTTTGCTGGCACTTGAACTCAGAATCCCTGGAAGAGCAGTCAGT
GTTCTTAACTGCTGAGCCATCTTTCTAGCACAATAATACATTTTTTATAAACAACAAAAAATAGATATAAAGTGGCAAAAAAGACATTTTATAT
AAGAGTTTAGTTGGCTTGTTTTGCCTTGAGAGAGGAATATTATTGTTAATTATACTTTTTAAAGTTTGTGCTGTTTTTTTTTTAAATATATAGC
TGTTACAAAGTTCAGAATTTAAGCACACAAGAAAGCCCAGCTATCAAAAAAAAAAAAAAAAAAAAAAAAAAAAAAAAAAAAAAGCCAGGCAGTT
GGGGCTCATGCTTTTTTTTTTTTTTTTTTTTTTTTTTTTTTTTTTTTTCCGGTTTTTGAAACAGGGTTTCTTTGTATAGCTCTGGCTGTCCTGG
AACTCACTTTGTAAACCAGGCTGTCCTCAAACTCAAAAATCTGCCTGCCTTTGCCTCCGAGGGCTGGGATTAAAGGGGGGCCCCACACAAAAAA
ACCCTCACTCGAAAAAACAAAAACAAAGAAAAAGAAAAAAAGCCCAGCTATAATATTCCCATCAACTTTCTCTCCCAAAGCCACTCATTTAAAG
ATTTACTTTATCTTTAAAATATTTGTACATGTACAGATAATATTTGTGTTATCTGTATCTGGCGCCCGCACGCGTGTGTGTGTGTGTGTGTGTG
TGTGTGTGTGTGTGTGTATGCCCAGGTGCTCCCTGGGTCCAGCAGAGGGAGTCTGATCGTTAGAGCTTGAGTCCCAGGCAGCTGTGATAGGGTT
GCTGGGCTCCTGGGAACTCAACTCGAGGCTTCTGCAAGAGCCATGCTCTTAGCCCGGGAATGGCTTCTCCAATCCTTTAGACATTTACTATGGT
AGGAGAGGTTGTCTCCATGTGCGTGTGCACATGGTACACACATCGGCGGCCTGGGGGTAGTTCTCCTTCCCCCATCCTCAACQACCAAGCTCAC
TGAAGCTCATCACCAGGTGCCCTTCCCACTGAGTGTCTCTCTCCCCTCCCAGAGCATCTTTCTCTATAGAGTCCTAGTTTTTCCAAATGAAGAA
TAACTGCTTTATTTTTTTCAGTCAAACTCTCATATCCCAGTTTGACTTAAGACTTGCTTTGTAGCTGAACTTCTGGTCCTCCTGCCTCCACCTC
CTGTTGGTGGTTTGTTTTATTTGTTTGTTTGTTTTTGTTTTTGGTGGTGGTTTTTTGAGAGAGGGTTTCTCTGTGTAGCCCTGGCTGTCCTCGA
ACTCACTTTGTAGATACGGCTGGCCTCAAACTCAGAAATTCACCTGCCTCCACCTCCCGAGTGCTGGGATTAAAGGCGTGTGCCACCACCGCCC
GGCTGTTTTGTTTTTAAGATAGGGATAAAATTCAGTATTTTGTATATACTAGGCAAGCTAAGATAAACCCCAGACTTTGCATATGTATTCTTAA
AATATGTACATTTACGACTGGAAAGGTCGTCAGAGGCTGTAAGAGTACTCACTGTGCAAGCATATACGGCCCCAGTTNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NTATATATATATATATATATATATATATATATATATACACTAAATAGAGAAAGATTTGGACATAGTTATTATGTGAAAGCAGCAGTCTTTCCAC
GTCAGATGAGTGCATTCTACACTTGTCCCTCACTGAGCTTGGTGTGAAAAGACTCACCTCTTTAGATCCATTCTGTGAACCATGGGGTTTCACT
TAACTGATCCTCACACGAAGATTTTCAGGCCTTGTTTTTATTATAAGCGTCATAATAATAACCATAATAATAATAAAGGCAGTGGTGGTGCAAG
CCTTTAATCCCAGCACTTGGGAGGCAGAGGCAGGCGGATTTCTGAGTTCGAGGGCAGCCTGGTCTACAGAGTGAGGTCCAGTCAAAGCTCTCAG
CACCATCACGQGCTCAAGCTTCACCCCCGAGCTGCAGACATTTTATACTAAAATACTGACAAGTGGGTGGACCGTGGTTGATTGTTGCGTGACT
CTTGTGCATAGTTCTCCTCACCTCCGTGTGATTGCGCTGCCTCAGCACTGAGGTGACAGGAGCGGCAGTTGTCCCTTCCAACCTGTGACCTCCC
CACCTCTCCTTCTGTTCTTAGGCCCTTGGACTGGACACTGCCAATGAGAAAGCCTTCTACAGAAGCCGCCAGCCCCAGCTGCTCATGAATGACT
TTGAGTCGGCCAAGGGCGACTTCGACAACGTGTTGGCAGTCAATCCTCAGAACAGGGCCGCTCGCCTGCAGATCTCCATCTGCCACAGCAACGC
GAAGGAGCACAACGAGCGGGACCGCAGGGTGTACGCCAACATGTTCAAGAAGTTCGCAGAGCGGGACGCAAACGTAGTGTGCCCACAGCCCCAG
GCTTGTAACCACGCTAGCCTAGCGACAACCTTCGCCACCCTCGCTCGAACAAATAAGACTTAGGAAGQGACTCCATGTGGGCAGAGACACACGT
GGGAGGCCCTGGCGTCTCCCTGAGACCTCAGCCCTGACTCCTGAGGTGGAGCAGTGCTCTTCTTGTTCTCTGTTTTGTTCTTAAAATGGTTTCT
CTTTGTGGACCCAGCTAGCTCATCTCAGAGATCCTCCTGCCTCTGGCGATGAGCGTTAGGATTATAGGCATCTGCCACCAAACCAAGTTCTCTT
GTTATTTTTAAAAAATAAATAACGAGTTACAGAGACAAAGTGTGGAGCAGAGACTGAAAGAATGACCATCCAGAGACTGCCCCACCTGGGGATC
CATCCCATAAACCACCACCAAACCCACTATGGCAGATTCCAACAAGAGCTTGCTGACAGGAGCCTGATATACCTGTCTCCTGAGAGGCTCTGCC
AGTACCTGACTAATACAGAAGTGGATGCTCACAGCCATCCATTAGACGGAGCACAGGGCCCCCAATGAAGGAGCTAGACAAAGTACCCAAGGAG
CTGAAGGGGTTTACAGCCCCCTGGGAGGAACAACAATATGAACTAACCAGTACCCCCAGAGCTCCCTGGGACTAAACCACCAATCAAAGAAAAC
ACACGGAGGGACTCATGGCTCTAGCTGCATATGTAGCAGAGGATGGCCTAGTCAGTCATCAATGGGAGGAGACACCCTTGGTCCTGTGAAGGCT
CTATGCCCCAGTATCGGGGACTGCCAGGGCCAGGAATAGGAGTTAGGTGGGTTGGGGAGCAGGGGGAGGGQAQAGGGATAACCGAATTTTCAGA
GGGGAAACTAGGAAAGGAGATAAAATTTGAAATGTAAATAAAGAAAATATCTAATAATGAATTAGTTAAAAATAAATAAATGCAGCCAGGCAGT
GGTGGCGCACACCTTTGATCCCAGTGCCTGAGAAACAGAGGCCAAGGTAGGCAGTTGTCTGAACAGGAGGCCACATACACACAGAAACCCTGTC
TGGAAAAAATGACAACAGCCCTAAGCCCTTCAGTGTTGTTTCTCTGGGGAGACTTCCTAGTAAAGTCCAGGTTGGATTGGAAACCGGTGTGCCT
GTGGTGGCCTTTACTGTATTCTCTGCTGTCATCGGGAGCCTCGCTGGCAGTGGGTGTGTCTGAACACAGGTCAACTTCCTGCCCCATGGCCACC
CTGTGGTCCCACGTCTGTCCTCCATCTTCAGCTATGAGTAGCATAAGGACTTGAGCTACTCAGCAGCTGCCTGAGGTAGGCAGCTCCTCATTGC
TTCCCACCCCTACTTACCCAGTGTGCACCCCAGATCCAAGCGTACACACTTGCTGCAGACCTGATGGACCATCTCGGGAAGGCAGTGGTTGAGT
GAGCTCCTCTTCTCCTTTCACCTCAGGAGGAAGCCAGCAAAGCTGGGAGCAAGAAGGCTGTAGAAGGAGCCGCTGGCAAACAACACGAGAGTCA
GGCCATGGAAGAAGGAAAGGCCAAAGGCCATGTATGACGCTGCGCCACGGAGGGAAGAGAGTCCTAATGAACTCGCCCCTCCTCGCTGGGCTCG
CCTCCAACTCAGGACTGAACAGTGTTTAGTGTAAGGTTTGTTACAGTCTCTGTGATTCTGGAAGCAAATGCCATACCAGTAGCTTCCCAAATGA
CCACCTGCTGCTGCGGGGGGGTGGGGGTGGGGGACATGCCAGGAAACAGCAGAGAAGGCCGCTGGTGTGAAGAGACCAGGCCAGCAGCTCAGTC
CAGCCCATTTCAGTTTGTCACCTTTCAGTGTCCAGCACAGCATCCCTGTGAACCTAGGGCCCAGCTGCTGTGGGTTCTACATCGGCACTAGGGT
CACACTGCAGAAACCGTTGATAAAACAAACTCAGTGATCTCTGCTTTCCTATTGGTGGGCATGGCAGGGGCGGGTGATGAGATTTGCTTAGCAC
TGACTGACTGGCCTGCTAAGAACACAAGCCCACAGCCAGGGGCTCCCTGGTCCACAGCTGGGTCTCAGGCCCCTTACCTGCCTTCCAAGTCCTT
TCGCAGACTCTTGAGTGTGGCTTTCTGTCCTAGCCAGCATGTCCCACAGACTCTGTTGTTCCTCCAACGCCCGTCATTAGTGACAGCTTTCTCT
CTGAGTTTCTCTGGTGTGGAGAGTGGGTAGAAGTAGGTTTATCTTTCCCGCTGTCTGCCCCACTCAAGGACGATGTTAGCGCTCAGCCATCCCT
GCCCAGCACACCTCCTCAGCCTCATGCTCCCACCCGCACCCTTGCTGTCCACAACCTAAGCCTTGCTCCCGTCCCAGACTCGAGTGGACGCACA
TCCATGTCCACCCAGGATTGACGAAGGAAGGAACTTCACACTCCTCTATCATGCAAAAATAGTTTTCAATTTTTACTTTTTAGCTTGGGTTTTG
AAAGGCAAACTCTAGGAGGCTTAAAACGGGCAGTTACTCTTAAGGCGAGGTAGATACCTCATCCTGCACCGCAGCCTCTGTATTCTGGTGCCAC
CTGTAAGGGTTGCCGGCTTCCGGTGTCCCATGCATCAAGCCAAAGTCTCCAAAACTTGGGCATTGAATTACCGCAAATCTTAAATAGTTTTGGG
GTTGGATTTTAGTCCTACAGGTGATGATTGAACGTGGCTGGCTTCTCCACAGCCTAAGGAACCCCCATGGGGACACACCCACCACCCAATGACC
CATGCACCTCTCCCTGGACAGCTCACTTCCAGCCCTAAGGACCACTGTGCTCAACATGTGTGGCTGTAGTAAGACAGTCTCATGTCAGGCATTA
AGTCAAGGCCTTCCTTCACACCTGCGGGACAGCGCTTCTGAGGCTCCAGTTCTGCTCTGCTCGAAGCAGGAGGGATGGGTTGTGAACGACTCTG
GGATGTGACTGGGACCCTCGCATCTCACACTCGGCACTCTGCCTCTTTCCATCCATGTTTGAGAGCCCTGGTCTCACCAGGAAGTGCAGAGCAG
GAGCCGACTGTGTGTGTAATGCTCTAGTGTTTGTAGTTCTCTTCACTACCCTTGCAGCGCCAGGGAGGCCGGGCTGGAATTTGAGCCTAACATG
TTAGGTTTTTACGGGAAAGCTCTTTTTCCAGCTGTCTCCCAAGTGCGTATTGTGGGCCAAGGAGACTGGTATGGGAAGGGGGGATGGTCAAGGT
TCCAAGCCAGAAATGGAAGGTGCCCAGCACCCCTGGATTTTACATGTTCTTGTATTCAGTAATCTCAGACGATTTACACGGAAATCAGAAGATG
TCTTCAAATACCCTTAGACTGGATAGAGTGATGGGGAAGCAGATGATGCATTGAAATTTTGGTTTTTAAAAAATCACTTAGATGCTGGGAGTAA
TGGTGACACACACCCTTGGTCCCGTGACCTGGGAGGCCCTGTCTCAAAACAAAACAAAAAATCATTTACATCCATTTTGTGTTCCTTAAATAAA
TTTTTTAAAAAGTGGTTTTTCATTGTTCATGGCTTTTTGTTTTTGTTGTTTGTTTGATTTTTGAAGTTTACTTTTATCTTTGTCTGCTAGAGTT
TTGTGTCCAGCTGCCCTTGGGGACCAGAAAGGGACTTTGAATCTCCTTGAAATGAAGATACAGTTGTTAGCCAGATGTGGGTGTTGAGAACTGA
ACCCAAGTCCTCTGAAAAGCAGGAAGTGCTCTTAACTGCTGGGCTGCCCCTCTCCCACCCCTCCATTGTTTGGGTTTCACTTAAACACTTGATT
TTPTTTATGATTTTTTTAAACAGATTTATTTATTGTATGTATGTGAATACGCTGTTGTTGTCTTCAGGCACAGCAGAAGAAGGCATTGGATCCC
ATTACAGATGGTTGTGAGCCACCATGTGGGTGCTGGGAATTGAACTCAGGCCCTCTGGAAGATCAGTCAGTGCTCTTATCCACTGAGCCATCTC
TCCAGCCCCTTTTTATGATTTTATTTTATGTGTATGCACACTTTTTTGTGATGCTTTATTGAGTTCCTATGGAACCCTTCTAACCCCCTACCTC
AGGTAGGCGACAAAGAAGGTTAGAGGGGGGAAGGGGGCATAGATCTTTTCAGACTGCTTCTTGCTGATTAGGGGCATCGAGTTCCTTGGGTCAA
CTTCAAACTTCATTGTCAGAATATCCAATCTACTCGTCTCTTTGCTCACAGCCTTGTGACAAGCCACAGCAGCAACAGACACAGTAGCCTCCTC
TGCAGAGCGCACTCCCACCTGATAGCCCCTCTCAGAGCTCGGCATTTATACCCTATGAGAAGTTCCCACAATCCCAGACTTCACAGAAACTGTC
TGCAGCTGGCAAAACCACGCCTCTCCCAGAGCACGAGGCAAATGATAGTCAGCTGCTGTGAAGCAGTCACCTATCTCACAACATAGTCACATGA
CATACCTCGGTTTTTTAGGAAACCGAAATTCTCACATGTTTACCTGCATGCATATACATACATCATGTCCATGTATTCCCCAGGGAAGGTAGAA
GAAGGCATCAGAGTCCATGGAACTGGAGTTACAAGATGGTTGTAAGCTGCCGTATGGTTCTTGGAACTGAACCCACATCCTATGCAAGAGCAGC
CAGTGTTTTTAACCACTGAGCCATCTCTCCAGCCCCTGATTAAATAGTATAAAAAGGAAAAAAAGGTCTACAGCCATCATGCCCTAAACCCACT
CAATCTCCACTGATCTCAGAAGCTAAACAGACTGGCCTTAGTCTATTCCTCTGATAACACACCATGACCAAGGTAATAATATAAGGAAAAGATT
ATTTGGAGCTTGCTGTTTCAAAGTTAAGAGAACCCATGTAGGGGGCTGGAGAGATGGCTCAGAGGTGGAGAGCACTGACTGCTCPTTCAGAGGT
CCTGAGTTCAATTCCCAGCAGCCACATGGTGACTCACAACCATCTGTAATGGAATCTAATGCCCTCTTCTGGCATGAAGGCATATATGTAGGCA
GGCAGACTATTGTGTACATAATTAATAAATAATCTCTCTTTTCTTAAAGAGTACATGGTGACAGGCAAAGTGGAAGAAATGTCAGAGAGAGCTA
TCTGCGAATAACATGAGTTTGTCAAACCTTCAACCCTATTCCTAGQAACACCTCTAACAAGCCACACCTCCTAATCTTTCCCAAGAAGTTCCAC
CTAGTAGAATTCATTGAAGAATGTGGTTGAGGGGCTGGAGAGATGGCTCACCAAAGGTTAAAGAGCACTAGCTGCTCTTCCAGAGGTCCTGAGT
TCAATTCCCGGAACACACGGTGGCTCACGACCATTTGTAATGGGATCTGATCCCCTCTTCTGCTGTCTCTGTAGAGAGCTACATGGTGTACTCA
TATACAATAAATAAATAAACAAACAAACAGACAAACAGACCAGTTCTCCAACTGAACCAAAGTGCATCCATTTTGCTGGGCTGTCTGGCCAATA
AGTATTCAGGGACCTACCTATCTACATAACCCCAGTTCCCCACTTCCAGATGTGGTGCCCTCTCTACCCAGCTGTGTCTGCACTATGCCGTGGA
TCCTGGGGATCCACTCGGGTCCGTAGTCTAAAGTAGGAGCTTTCCCAGCTGAGCCATCTCCCCTGTTCCTTACTTTGCCTTTTTGACACACAGT
CTCCACATTTGTCTGAGTTTCCACTCTCCAGACTTTGCCTCCTGTTTGCCAACCACCTGGTTCTCTTGGGAGACATATTGAGGGCTTACATAAT
TTGCATAATGAGATGAAACCTATCTACCCTCAACAAGCATACCAGTTAAGCATTGCTGAGCCACACTGCCCCAACCAGTGGAGACACATGCTCA
TGGTTGTTGGGAACTGCTGGAAATAGAAGGGTACTGAACCACAAATGCCCCATCTCCTTGTGTGCACAAAGAGCAAACATTTGGCTTCATAAAT
TGATTTTGGTTTAGTTTGGTTTTGCGTTTGCACTTGAAATTAAGAACTAATCCCTCTTTTTGGCACGTGTGTGTGTGTGTGTGTGTGCCTGGTA
CGGAGGTGCATTTGCACAGGTGTGCTTGGGAGTACAGATCAGAAGTTCACATCAGGGCTGGATGATGGCTCAGCGGTTATAGGGCACTACCTGT
TTGCTCTTCCAGAGGTCCTGAGTTCAATTCCCAGCAACCACATGGTGGCTCACAACCATCTGTAACGGGATCCGATGCCTTCTTCTGGTGTGTC
TGAAGAGAGCAACAGTGTACTTACAAACATTAAACAAGTAAATTGTTAAAAGAAGAAAGGAGGGAAGGAAAAGAAAGAAGGAATGTCTGCCTTT
TTCTTTGTTCCCAGCTTATACATGCAGACAGAGCCTCTCCCCCTGAATCCACAACTTGTCAAGTCAGCTCAAACTAGCCATCTTGCACGGAGGT
CCCCTGTCCCCACCTCACAAGCACTGGGGTCACAAGGTGGGCACCCTTGCACTTTCTTCACTATTTTCGTTTTTAGTTCCATTTGGACATGAGT
AGCCTCTCTGTTTTCTTCAATGCAAATACTGCTGCCTGGTGTAAAAGGCACTTCCTGTGCCCTTGGAGGCACAGCTTTCTTCCCTGAAATCCCA
GACGCTGCCAGAGACAGACAGACAGAGAGAACGCTCGCACACAGGATCACACATGCACACCAGTGAGTGCAGGTCTCCCCAGGGGCCAACAGAG
AGCATGCGATTCCCCGGGGTTACAGGCAGTTGTGAGCTGCCATGTGTGCTTGCTTGGGAAATGAATGTGGATCCTTTGAAAGAGTAATACCAGC
TCTCCATTGCTGAACCATTTTCCTGTGGGGCCGTGGTCCGCGAGAGGAAGTCTGGTAAGGTCGAGCTGGGCTTGAAGCTCCAGGGACCCAGAAG
GGACAGTAGGAGCTTCACCTGCTCCCAACATCAGGCCCCTGTCACTGTCACAGCCACCCATAGAGAGATTTGTGGCCATCAGTCACATAGGCGC
AGCACCCCCCAACCCCTCCACATGTAGATGAGGCATCCCTATACATCTCAGACCAAGCCAATAGGAAGCACCTCCTGTCAGACCCCACCCCCAC
CCCAAAACGGTGCATAATATCCTATCCAGAGGAATAAAGGTCTGTGTGAGAGTTACTCCATCATCTGGGAGCATCTGTCTTAGATGAGCTGCTG
TTACACTGCCTGCGAAGAACAGCAGCAQCCAAGGCAGCAGCAGCAGCAGCAGCAGCGCATCGGTGGTGGCTGCGGGCAAGAGGTCTCTGCAGAT
AGACTTAGGTACGCAGACCAGCAGTTCCCTAGCCCCAAGATCTTGTCTCTTTTTAATACAGACTTATCGGCCCTATCTTCCCTTCTGGCTTGCC
ACAGCACATATGCATTGGTATGGCGATTTTTGTTTTGCCTGCACGTGTTGATGTATTTAAAAAAAGATAGAGAGAGAGGGGAATATGGTCTATG
GAGATGTGATGAGGTATAGGGCAGGGGGGTGCCTCCTCCAGCCCATGGCTGGCATCCCTTCAGAACTGAGTGACCAGCCACATGATGGTATAGT
ATAGAATAGAGTTTATTCAGGGCATGGGGAGGGGAGTTAAAAGGGTAGTAGAGGCAGAGAAAGGCAGAGAGAGGGAGAAGAGTAATAGGGAGAA
GTAGAATAGAGGCCGGCCATGAACACATGGACAAATGGGGAAGGGACTAGGGAAGGGAGGGGAGGAGGGAAACGCCTACGAGGGCAAGAGAGAA
ACAACAGAACAAGGAGGCGGCAAGCAGCTCCTTTTATAGTGAGCCAGCCCTACCTGGCTATTGCCAGGTTACTGTGGGGTGGAGTTTAGACAGA
ATGCTAACACGTACGCCTGTGCACCACCTCTGTGGACCACATGTGTACCCAGTGCCTCTGGAGGGCACATGCGAGTCAGATGCCCCAGAGCTGG
AGTCACAGGCAGTTGTGAATGCCACATGGGTGCCAGGATTGAACCCGCGTCCTTTGCGAGAGCAGCTGGTGCTTGTATAGCGATTGAGCCGGGT
CTCCAGGCTGCATTTTTTGTCTTGATTCATCCCAAGGAATTTTCTAATTTCCTTCATAATTTTTTCTTTCACCCTCAGCTTGCTAAAGAGTCGG
CTTCCTTCCCACATATTTGTATCCTTTTCCAACTGCCTGCCTCCCTTCTGTTATTAATCTGATTTCATTCCACTGTGGTGAGAGTGCACACCAT
GTACATTCTTTGTTTGACTTACGTTATTTTAATATATTGAGACTTTTTGCGGCCACATATGGTCACCCCGACCCTCCCCCCGGAGATATCCGTC
CTGCTTTTAAGAAGATCTCATACATGTTTTTGCTTGGCATTTTAAAAAGCTTTTAGTTTCACATTTTATATTGCTTGCGAATATACATGTGTGA
GTGTGCCTGTCAGCAGACATGTCTAGGTCAGAGGACAACTTGGGGAATCCACCCCCTGGCCCAGGGTGGCATTATTCGCGTGTGAGGTCGGCTG
GGTTTCCAGTGCATGTGGCTCGGGCTTGTCTGGTTCCTTTCTGGACGTATGGTATTTGCAGCCTCTGTCCTGACTGATGATACTAGCTTGATTT
GCCTTTGCATTCTCCAGCTTGGCTTCCTATATTTTGGGGCTTGGTGTGCACACGTGTTTCTAATAATATCTTTCTCATGGGTTAACCCATTTCT
TTTTCTTTTCTTTTTTTTTTTAAAGATGTATTATTATTATACATAAGTACACTGTAGCTGTCTTCAGACACACCAGAAGAGGGTGTCAGATCTT
GTTATGGAATCGTTGTGAGCCACCCTCTGGTTGCTGCGATTTGAACTCAGCACCTTCGTAGACCGATCAGTGCTCTTACCCGCTGAGCCTTTTC
ATGGTGAGATCTTGGGTTCATCCTTCAAGAGTGCTTGCTGCTCTTCCAGCAGACCCATGTTCAGGTCCCAGCATGCCACATCAACACCTTACGC
TGCCATCTCAACTGCTCCCCCGACTCCAGTTTGCACGTGTTCTCTCTCACTGCTCTGCCCTGTTTGTCATTCTTGGCCACCGCCTTAGTTTGTG
TTACATAAATATTTTCCAGTGTGTCATTCCAATTTCCTTATCTTTTAACATCTTAAATTTTTTTTAAAGATTTATTTATCTTATATATGTAAGT
ACACTGTAGCTGTCTTCAGACACACCAGAAGAGAGCATCAGATCCCATTACAGATGGCTGTGAGCCACCATGTGATTGCTGGGAATTGAACTCA
GGGCCTCTGAAAAAGCGGCCAGTGCTCCTAACTGCTGAGCCATCTCTCCAACCCTTAACATCATAATTTAATAGGATATAATTTGAGCCCATAC
CGATGTAATTTCAATAGCCAAGCATGGGGGGCAATGATGGTGCACGCTCTTAATCCCACTGCAGAGTCAGAAGTAGGCAGATCTCTGAATTCCA
GGCCAGGTCTGTGTAGTGAGTTCCAGGACAGTTGGGGCTACACAGAGAAACCCTGTCTGGAAGAAAACAAACCAAACAAACAATCACACACACA
CACACACACACAAATTGTTAGCCAAACATAGTAGCAAGTTCCAGTCCTTCAGATAAGAAAGCAGGAGGGTTAGAATTTCAAGACCAACCTGGGC
TGCATGAAACCCTATCTTAGAAAAAAATTAATCTATTTCTCTATCACTCCTTTCTCCCTATCGCCCCCTCTGACACCGTGAGCCGGCCAGAGCA
TAGTTTTTCCTTTGTTTCTTTGGGTACATATGCTGCTAGCTCCTAGCTTATGAGCCATCTCTCTATTCCTCTCTCTGTCTGTACACCTGGATTT
GATTTCTGTCTTTGGTGTTCCTGCCCCCCTTCTACCCTTCCCCCCAGCTATGGGGTTTGGGTTGGGGAATGTGTTTTTTATTTTATTGTTTAAG
GTTTATTTAATATGAATACACTGTAGCTGTCTTCAGACACACCAGAGAGGGCGTCAGATCTCATTAAGCTGGTTGTGAGTCACCAATAATGCTT
GCTGGGAATTGAACTCAGTACCTCTGGAGAGCAGTTAGTGCTCTTACCTCTGATCCATCTCTCCAGGCCCCTTGTGTTTTTTTCTTTTTGTATC
TGAATTTCAACAGTGATCAAATCATTTGCTCCTTTTTATGGAGTTTAGATCCTTGCAAAAGATCTGGGGGCAGGAGGGTTTTATTACCATAAGG
CCTTGCTGGGGGACACCAGGAAACAGCTTTGTTTTCTCTCGGAGCAGCTGCTCACTCTGACTCCCCTGCCATTGTACATGGGTTGCTTACACCA
GCTCTCCTCTGCTCCAGGATTCCGTGCTCCGGAAACCGCAGGGAGGGCCCAGGCAGCCATAGACATGGGGTCAGCTTCACCAAGTTAATGCAGG
AACCCAAGAGTCTCAGAAGCGCGGCTGTGCCTCCCCCCCCCCCCCCCGCCTCGCTCCCCTCCACCCCCCAGGCTGATTTAAGCTCCCAGCAGTG
AGCCTCTGGGCAGCTGGTGAGGCTTCACCAAGTCAATGGAGCCTCAGCCACTGTCCACCTGGACGGGAAGAGGGTCCCCACCTCTCTCTGCCCT
TCAGCGAGATCAGTTCCACCACTCTGCCCTCTGCCGCAGCGGCAGGCGCGCGCCCACCAGTAGCCACCACGTGAGGCCCGGGCTCTGATGTATC
TTTCTGGACTTGCAGCTCAGTCCTCCCTGCCCTCCCCTGCGTGCCTGGCAGCTTCCTGTAATTCCAGGTAAGCACCCTCCACGAAGCCGCGGTG
TGCAAGCCTGCTGCGTGATCTGGTGACTGGCTAGGACGTGGGTGGAAGTCTGGAAGGAACCTGGGCCCTTTGGTTCAGCACAGCAAGGTGCCAC
TGAGCAGCGTGCTAGGGGTGGGGACATCAGAGAAGATGCAGAGTCACCTCTGCCACCCTCTTCAATTCGGACACCATTGCAGCCATGTGCAGGA
GGGGGACAGGGCAGAGATGCACCAGAACCTGGCTTTAGGAAAGAAAACAAAACAAACAAACAAAACCAAACCAACCAGCCCAGCTGNNNNNNNN
NNNNNNNNNNNNNNNNNNNNNNNNNNNNNNNNNNNNNNNNNNNNNNNNNNNNNNNNNNNNNNNNNCTGAGCCGGGCAGTGCTGGCGCACGCCTT
TAATCCCAGCACTTGGGTAGGCAGAGACAGGCGGATTTCTGAGTTCGAGGCCAGCCTGGTCTATAGAGTGAGTTCCAGGACAGCAAAGACTATA
CAGAGAAACCCTGTCTCGAAAAACCAAAAAAAAAAAAAAAAAAAAAAAAAAAAAAAAAAATCAAGCTGTATAGTGCTGGTGAGATGGCTCAGCG
GTTAAGAGCACCGACTGCTCTTCCAGAGGTCCTGAGTTCAAATCCCAGCACCCACATGGTGCCTCACAGCCATCTATAATGGGGTCTGACTCCC
TCTTCTGGTGTGTCTGAAGTCAGCTACAGAGTACTCACATAAATAAATAAACAAACAAACAGACAAGTAAATAAATATCAGCCTCCAGGGATGC
CAGCTCTGGAGGAAAACCTGACACAGGGCAGAGGCTGCAGCTCAGTTCTCAGTGTGCGACCAGGAAGCACAAAGCCCTGACTTCCACCCCTATC
CTCAGGAGGTGGAGGCAGGAGGATCAGTGGCCATCTTCAACTACATTGCTTCTTAGAGCCTGCTCGCCTTGAGACCCCATCTCAAAAATAAATC
ACTATAAAAAGAAAAGCCAGAGGCTGGTGAGATGGCTCAGTGGGTAAGAGCACCGACTGCTCTTCTAAAGGTTCAAATCCCAGCAACCACATGG
TGGCTCACAACCATCTGTAACAAGTATCTGACTCCCTCTTCTGGAGTGTCTGAAGACAGCTACAGTGTACTTACATATAATAAATAAATAAATC
TTTAAAAAAAAAAAAAGAAAGAAAGAAAAGAAAAGCCAGCCGGGTGTGGTGGCACATGCCTTTAATCCCAGAGGCAAGCGGATTTCTGAGTTCA
AGACCAGAATGCTCTACACAGTCAGTTCCAGGGAAGAAGAGAGAGAAGAAGAAGAAGAACAAGAAGAAGAGGAGGAGGAGGACGAGGAGGAGGN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNACGTGAATAGGGCGGGCAGAACCTGGAGCCCGCTGGCCTCAAGATTGAGCGGGATTTTGGTGGATGCGTTTTGTGGTCTGAAGG
CACCCTGCTGGCCGGTCCTGGCCTGCCCTAAAGGTGACCGGAAGAGAAACGAAGAQTTQGGGAACACTTCCGGTGCGGGGGATAGTAGGTACAG
AAGCTAACTGACCCCTGGAGGATTTGCTGTTGATCCTGGCACCTTCCCTTTCCCCCAGGCTGAGCTCTGCCCTGCAGCCTCTTGCCCCAGTGAC
CTCGTTCTAGAACTAACCTGGCAGGTGGTCCATCCTGTAGTGAGGGAGGCCCAGCTTGGAGGCCATGCCTG
MOUSE SEQUENCE—mRNA
GTCCGGCGCGCGGCTGAGCGACCGAGCGTGCGGACGGAGCGGCGGCCTGCTGGGCGGGCTGAGCGGCGCGCGCGGCGGCGGAGAGACCCGGAGC
GAGGGACGCGGCGGCGGCGGACGCGGCGACAGGTCTTCThCTTACAAAGGACAATGACTACTGATGAGGGCACCAGTAACAATGGAGAGAACCC
AGCAGCCACCATGACTGAGCAGGGTGAAGATATCACTACGAAGAAAGACAGAGGAGTATTAAACATTGTCAAAAGAGTGGGGACTAGTGACGAG
GCCCCAATGTTTGGTGACAAAGTTTATGTCCACTACAAAGGGATGTTGTCAGATGGAAAGAAGTTTGATTCCAGTCATGACAGAAAGAAGCCAT
TTCCCTTTAGCCTTGGCCAAGGCCAGGTTATCAAACCCTGGGACATTGGGGTGTCTACTATCAAGAAAGGCGAGATCTGCCATTTATTATGTAA
ACCACAATATGCTTATGGCTCGGCTGGCCACCTCCAAAAAATTCCATCAAATGCAACTCTCTTTTTTGAGATTGAGCTCCTTGATTTCAAAGGT
GAGCATTTATTTCAACATTCAGGCGTTATCCGTAGAATCAAACGGAAAGGCGAGGGATACTCAAACCCAAACGAAGGAGCAACGGTAAAAGTCC
ACCTGGAAGGCTGCTGTGGTGGAAGCACATTTGATTGCCGAGATGTGGTGTTCGTTGTTGGGGAAGGAGAAGACCACGACATTCCGATTGGGAT
CGACAAACCCCTGGTGAAGATGCAGAGACAAGAACAGTGTATTCTATATCTTGGACCACGCTATGGTTTTGGAGAAGCCGGGAAGCCTAAGTTT
GGCATTGACCCCAATGCTGAGCTTATGTACGAGGTCACCCTTAAGAGCTTCGAGAAGGCCAAAGAATCTTGGGAGATGGACACCAAAGAAAAGC
TGACGCAGGCTGCCATCGTGAAAGACAAGGGAACTGTGTACTTCAACGCAGCCAAGTACACGCAGGCCGTGATTCAGTACAGGAAGATAGTGTC
CTGGCTGGACATCGAATACGGCCTGTCAGAGAAGGAGTCCAAAGCCTCAGAGTCGTTCCTCCTCGCAGCCTTCCTGAACCTGGCCATGTGCTAC
CTGAAGCTCCGAGAGTACAACAAAGCCGTGGAGTGCTGCGACAAGGCCCTTGGACTGGACAGTGCCAATGAGAAAGGCTTGTACAGAAGGGGCG
AGGCCCAGCTGCTCATGAATGACTTTGAGTCGGCCAACGGCGACTTCGAGAAGGTGTTGCCAGTCAATCCTCAGAACAGGGCCGCTCGCCTGCA
GATCTCCATGTGCCAGAGGAAGGCGAAGGAGCACAACGAGCGGGACCGCAGGGTGTACGCCAACATGTTCAAGAAGTTCGCAGAGCGGGACGCA
AAGGAGGAAGCCAGCAAAGCTGGGAGCAAGAAGGCTGTAGAAGGAGCCGCTGGCAAACAACACGAGAGTCAGGCCATGGAAGAAGGAAAGGCCA
AAGGCCATGTATGACGCTGCGCCACGGAGGGAAGAGAGTCCTAATGAACTCGGCCCTCCTCGCTGGGCTCGCCTCCAACTCAGGACTGAACAGT
GTTTAGTGTAAGGTTTGTTACAGTCTCTGTGATTCTGGAAGCAAATGGCATACCAGTAGCTTCCCAAATGACCACCTGCTGCTGCGGGGGGGTG
GGGGTGGGGGACATGCCAGGAAACAGCAGAGAAGGCCGCTCGTCTGAAGAGACCAGGCCAGCAGCTCAGTCCAGCCCATTTCAGTTTGTCACCT
TTCAGTGTCCAGCACAGCATCCCTGTGAACCTAGGGCCCAGCTGCTGTGGGTTCTACATCGGCACTAGGGPCACACTGCAGAAACCGTTGATAA
AACAAACTCAGTGATCTCTGCTTTCCTATTGGTGGGCATGGCAGGGGCGGGTGATGAGATTTGCTTAGCACTGACTGACTGGCCTGCTAAGAAC
ACAAGCCCACAGCCAGGGGCTCCCTGGTCCACAGCTGGGTCTCAGGCCCCTTACCTGCCTTCCAAGTCCTTTCGCAGACTCTTGAGTGTGGCTT
TCTGTCCTACCCAGCATGTCCCACAGACTCTGTTGTTCCTCCAACGCCCGTCATTAGTGACAGCTTTCTCTCTGAGTTTCTGTGGTGTGGAGAG
TGGGTAGAAGTAGGTTTATCTTTCCCGCTGTCTGCCCCACTCAAGGACGATGTTAGCGCTCAGCCATCCCTGCCCAGCACAGCTGCTCAGCCTC
ATGCTCCCACCCGCACCCTTGCTGTCCAGAACCTAAGGCTTGCTCCCGTCCCAGACTCGAGTGGACGGACATCCATGTCCACCCAGGATTGACG
AAGGAAGGAACTTCACACTCCTCTATCATGCAAAAATAGTTTTCAATTTTTACTTTTTAGCTTGGGTTTTGAAAGGCAAACTCTAGGAGGCTTA
AAACGGGCAGTTACTCTTAAGGCGAGGTAGATACCTCATCCTGCACCGCAGCCTCTGTATTCTGGTGGGAGCTGTAAGGGTTGCGGGCTTCCGG
TGTCCATGCATCAAGCCAAAGTCTCCAAAACTTGGGCATTCAATTAGGGCAAATCTTAAATAGTTTTGGGGTTGGATTTTAGTCCTACAGGTGA
TGATTGAACGT
MOUSE SEQUENCE—CODING
ATGACTACTGATCAGGGCACCAGTAACAATGGAGAGAACCCAGCAGCCACCATGACTGAGCACGGTGAAGATATCACTACGAAGAAAGACAGAG
GAGTATTAAAGATTGTCAAAAGAGTGGGCACTAGTGACGAGGCCCCAATGTTTGGTGACAAAGTTTATGTCCACTACAAAGGGATGTTGTCAGA
TGGAAAGAAGTTTGATTCCAGTCATGACAGAAAGAAGCCATTTGCCTTTAGCCTTGGCCAAGGCCAGGTTATCAAAGCCTGGGACATTGGGGTG
TCTACTATGAAGAAAGGCGAGATCTGCCATTTATTATGTAAACCAGAATATGCTTATGGCTCGGCTGGCCACCTCCAAAAAATTCCATCAAATG
CAACTCTCTTTTTTGAGATTGAGCTCCTTGATTTCAAAGGTGAGGATTTATTTGAAGATTCAGGCGTTATCCGTAGAATCAAACGGAAAGGCGA
GGGATACTCAAACCCAAACGAAGGAGCAACGGTAAAAGTCCACCTGGAAGGCTGCTGTGGTGGAAGGACATTTGATTGCCGAGATGTGGTGTTC
CTTCTTGGGGAAGGAGAAGACCACGACATTCCGATTGGGATCGACAAAGCCCTGGTGAAGATGCAGAGAGAAGAACAGTGTATTCTATATCTTG
GACCACGCTATGGTTTTGGAGAAGCCGGGAAGCCTAAGTTTGGCATTGACCCCAATGCTGAGCTTATGTACGAGGTCACCCTTAAGAGCTTCGA
GAAGGCCAAAGAATCTTGGGAGATGGACACCAAAGAAAAGCTGACGCAGGCTGCCATCGTGAAAGAGAAGGGAACTGTGTACTTCAAGGGAGGC
AAGTACACGCAGGCCGTGATTCAGTACAGGAAGATAGTGTCCTGGCTGGAGATGGAATACGGCCTGTCAGAGAAGGAGTCCAAAGCCTCAGAGT
CGTTCCTCCTCGCAGCCTTCCTGAACCTGGCCATGTGCTACCTGAAGCTCCGAGAGTACAACAAAGCCGTGGAGTGCTGCGACAAGGCCCTTGG
ACTGGACAGTGCCAATGAGAAAGCCTTGTACAGAAGGGGCGAGGCCCAGCTGCTCATGAATGACTTTGAGTCGCCCAAGGGCGACTTCGAGAAG
GTGTTGGCAGTCAATCCTCAGAACAGGGCCGCTCGCCTGCAGATCTCCATGTGCCAGAGGAAGGCGAAGGAGCACAACGAGCGGGACCGCAGGG
TGTACGCCAACATGTTCAAGAAGTTCGCAGAGCGGGACCCAAAGGAGGAAGCCAGCAAAGCTGGGAGCAAGAAGGCTGTAGAAGGAGCCGCTGG
CAAACAACACGAGAGTCAGGCCATGGAAGAAGGAAAGGCCAAAGGCCATGTATGA
HUMAN SEQUENCE—GENOMIC
CAAAAACCAGCAAGTTTTTATTAGGGATTTTCAAAAGGGGAGGGAGTGTGTGAAGAGTTGTGGGTGACAGACATCAAGTACTTAACAGGGTAAT
AGAATATCACAAAGCAAGTGGAGGCAGGGCGAGATCACAGGACCACAGGACAGAGGCGAAATTAAAATTGCTAATAAAGTTTCGGGCACCATTG
TCATTGATAACATCTTATCAGGAGACAGGGTTTTGAGAGCAACTAGTCTGACCAAAGTTTATTAGGCGGGAATTTCCTCTTCCTAATAAGCCTG
GGAGCGCTATGGGAGACTCGAGTTTATTTCACCTCTGCAATCTCGACCATAAGAGACAGGTATGCCCCGGGGGGGCCAGTTCAGAGACCTACCC
CTAGGTGCGCATTCTCTTTTCTCAGGGACTTTCCATGCTGAGAAAAGGAATTCAGTAATATTTCTCCCATTTGCTTTTGAAAGAACAGAAATAT
GGCTCTGTTCTGCCCGGCTCACCGGCGGTCAGAGTTTAAGGTTATCTCTCTTATTCCCTGAACAATTGCTGTTATCCTGTTCTTTTTTCAGGGT
GCCCACATTTCATATTGCTCAAACACACATGCTGTACAATTTGTGTACTTAACGCAATTATTACAGGGTCCTGAGACGATATACATCCTTCTCG
GCTGACAGGATTAAGAGATTAAAGTAAAGACAGGCATAGGAAATCACAAGGGTAPTGATTGGGGAAGTGATAAGTGTCCATGAAATCTTTACAA
TTTATGTTTAGAGAGTGCAGTAAAGACAGGCATAAGAAATTACAAAAATATTAATTTGGGGAACTAATAAATGTCCATAAAATCTTCATAATCC
ACGTTCTTCTGTCATGGCTTCACCCGGTTCCTCCGTTTGGGGTCCCTGACTTCCTGCAACACCTTCTTATATGTATCAAAACTCATACTGAACA
ATGAGTTCTGGGTTGCAAAAGAGGATTTATTTTTTGCACCTAACCATAAGTCTTTGTCCACAAGTTAAACAAAAACAGTGTAGTATACAGTATT
TTTAATAATTTTGTGCATTAAATAAAATTTGTGTACCTTGAATCATTAGAAAACAAGTATTAGGTGTGGAGTTTTTCACTTGTGGCATCATGTT
AGAATTCAAAAAGTTTCAGAGTTTGGAGCATCTTGGATTTCGGATTTTTCCAATTAGGAATGCTCAACCTAATCAGAAGTAAGACTTGGATATT
AAAAGTTTGAAAGCCACGGTTTTATTTGCTTAGTAGTATATAACTGCAACAGTACTTTTTATCTGAATTATTGTGTAGAATCTGATGACTCCCC
AACATATAACTGTCACCCACACTTCTCTCCTGAGCTCAGGCCTTATCTCTGATAACCTTCTGGACATCGCACAAGCACCCCTTGCTCAACATAC
CCCAGCCAGAGTTCTCTTGCTCTCCATAAAAAGCAATCTGAAATGTTCGAATTAAATTGAAGAGAAAGAATAAAAAGTCCTGATGAGTGGAACC
ATGAAATGTTTCATCTTTTTTCCCTAAGTTTAAAAAGATCATGAAAGTAATACATGTTTAATGTTAAACTATTTAAATAATATAAAAATAAAAT
CTAAGAAGTGAAAATTCCTTCTTATCTTCCCAAATCTCATTCTCCAGAGGTAACACTGTTTGAGCAGTTTGGTAAGTTATCTTTCCAAACTTCT
TCTATATATAAGCAAATATACAAACACATATATACTTTTTTCCTCTTGGAAATAGGATCATGTTACAAATCATTCTGCAACTCACCATTCACTT
AAThGTACATCTTCAAAATCCATCTGTCTTTTCATGTGAGTATTAGAAGATTGTTTATGTGGAATGGGTAAGACGTCACCTGCTCCATATTAGT
GATATTTTCAGAGATTACCTGTCTTAAAGGGAAAACAGTGAAAAAGACATTTTGGCATGTAATCAACAATATTTGCATAATTGTAATCCAGTGC
TCTAAAATGATCATACCCACCCACCCACCAAAATCTACAAACAACTGGGAACCATGGTTGGTGGCTGCXAATATATTGGTATAAATTACACTGT
TTGTGCCAGGTGCGGTGGCTCATGTCTGTGATCCCAGCACTTTGGGAGGCTGAGGCGGGCGGGTCACTTGAGCTGAAGGAGTTCGAGACCAGCA
TGGCTAACATGGCGAAACCCCATCTCTACTAAAAATACAAAAATTAGCCCGGCATTGGTGGCGCACACCTGTAATCCCAGCTACTCAGGAGGCT
GAGGCAGGAGTATCATTTCAACCTAGGAGGCGGAGATTGCAGTCAGCCAAGATCAGGCCACTGCACTCCAGCCTGGGCAACACAGCAAGAGTCT
GTCTCAAAAAATTTAAAAAAATAAAAAATAAATTACACTGTTTCTGTTACATGTATTAGCCCATTGTGGCATCATCAACTGACTTTTTTTTTTT
TCAATGGTCCAGATAACCAAATCATCACTGGGCACTATGCACCATTAACAATACACTTCATTTACTACAATGCTATTCTTTTTTCCTTTTTTTT
TTTTTTTTGGACAATATCTTGCTCTGTTGCCCAGCCTGCAGTGCAGTGGTGCAACCATGGCGTGATACTCCTGCCTCAGCCTCTCCAGTAGCTG
GGACCACAGGCGTGTATTTTTTATTTTTAGCAGAGATCGGGTCTCACTGTGTTGCCCAAGCTGGTCTCAAACTCCTGGGCTCAAGCCTTGACAT
CCCAAAGTACTGGGATTACAGGTGTGAGCCACTGCGCCTGGCATTCTTTTATAATCGATGAACATTTACAAAATATTTTCCCAATTTAGTTGTT
TGGATGTTTTTGTTTAGACTCTTAGCTAAATGATTCTCTACTGTATAGATTTATCTCATGCTTTTCAAAAACTGTATATTTTTATAGTATGCAT
TTGCCAATTTTTAGTCTAACATTCCCATGACCATCCTTTTAAGGAAAAACTTTCAAATACTTTTATTTAGCCATACGGTTAAAATTTAGCTGGA
ACTAAGTGATCTGTGTTTAGTTGATGAACTGATCCATGCTATTCTTGCATCATGACTTTTTCATCTTCCCTTGATTCTTTTCCCTATATGCATT
TAAGCCTGTCTGGTTCTTTTCTCAAGCCTTTGCTCTCTACTCCCCTTTCACTCAAAGCATTTCATGTTTCGCCTTTATAACCCTAGTATAAGTA
TCAAACTTCTCACCTGTGACTTTTGCCTCTCTCTACATCTCTCPTCTACTTTTAACTTTCATGTTTACAATAAGCTACCTTTCTCTTATTTTCA
GTTCTTACCTTCTCAAACTTCAGAGAATCCCCACTGCTTAGTGTTCTTGGGAGATGCTAGAGGTTACACAAAATGCCACCTCCCAGAAGGTCGA
GGCATTTTTTTTCTTTAAAAAAAAAAAAACCTTTTAGCCAAATACTTACTCATAAGAAATGCTCCATTTTATTCTCAGTAGTATCTTCAGAAAG
ATTTCAGAGGTAATTGCATCTACAAAATAAAGACTTAGTGCTAAGAAAATGAGCAATCCGATAAGAATATGTAAAATAAAAAACATTATTACTA
AATTCTTTTTTTTTTTTTTTTTTTTAGACAGAGTCTTGCTCTGTCGCCCACGCTGGAGTGCAGTGGTGCGATCTTGGCTCACTGCAAGCTCTGC
CTCCCGGGTTCACGCCATTCTTCTGCCWCAGCCTCCCAAGTAGCTGGAACTACAGGCGCTCGCCACCACGCCCGGCTAATATTTTGTATTTTTA
GTAGAGACAGGGTTTCACCATATTAGCCAGGATGGTCTCGATCTCCTCACTTTGTGATCCGTCTGCCTTGGCCTCCCAAAGTGCTGGGATTACA
GATGTGAGCCATCCCGCCTCGCCTTTTTTTATTTTTATTTTTTGAGATGGAGTTTCACTCTTATCCTCCAGGCTGGAGTGCAATCCCACAATCT
CCACTCACTACAACCTCCGTCTCCAGCGTTCAAGCGATTCTCCTGTCTCAGCCACCTGAATAGCTGGAATTACAGGTGCCCGCCACCATGCCCA
GCCAGTTTTTGTATTTTCAGTAGAGATGGGGTTTTTCCATATTAGCCAGGCTTGTCTCAAACTCCTGACCTCAAGTCATCTGGCTGCTCTGGCC
TCCCAAATTGCTAGGATTACAGGCTTGAGCCACCTCACCTGGCCACTAAATTCTTTTAAAAATCTCTTGAGCACACTAAATGATATATTGAATA
TAGCTGATGACCAAATGGTGTTGTAAAAATTCCATCAGGCCAGGTGTGGTGGCTCACTCTTAATAATCCTAGCACTTTGAGAGGACGAAGTGGG
AGGATCACTTGAGCCCAGGATCACTTCAAGACCAGCCTGGGCAACATAGTGAGACCCTGTCTCTTCTGAGAAAAATAATAAAAAAATAACATTA
GGTGTGGTGGCACATGTCTATAGTTTGAACTACTAGGGTGGCTGAAGGGGAGGATTGCTTGACCCCAGGAATTCAAGGCTGCAGTGAGCTATGA
CGGTACCACTGCACTCCAGCCTGGGCAACAGAGCAAGACCCTGTCTCTTAAGAGACAATAAGAAATAAATAAAAATTCCATCAAAGAATTTCTC
TTAGGCTATAAAGCCAGAGACAAAGATCAAAAATCGGAGAAATGTCCAGTGGTTCTCAACCATGTCTGACTCCACATTAGGATCATCTAACATG
GTTTTTGAAAACACCATTACCCTGGCCCCACCACAGACCAATTAAATCCAGACTCTCTGGGCTGGAGTCCAGACATCAGTAGCTTTTTTAAGCT
CCTCACGTCATTCTGTTGTTTAGCCCAGGTTTAGAAAAATACTTAAGAAACCTGAAGAATAATACCAATATCTCCGAACTCTATCTAATAATAC
TTCTAGAAGTAGAGGACAGAGACATCAAAGGAAAAGAAATAATGCTAGAAAATTTTTCTGGAGATTCTGCAAAGACTTCTCTTCAGCAGTTAAC
GGCCAGAATATCAAGQCCCCTGCAGGAATTAAGCAAAGAAGACAAGTACCTTGACATATCCTTTCGATGCGTCTCAACTCCAAAAATAAGGATT
ATTATGTCTTCCAGAGAAGAAAAGATGGGAGCCACAGTGTGCACTCTGGAAGGAAAGAAACCTACCTGGCCTAACTGATCCAAACACAGTTTCT
GCTTTTTCCCCCAGATGCTGCTCTCCAGTGCATTCCCCTAGCTCACTCCTGCTTTCTGGGGCTGACCCCACACCTTGAGCTTCTCCAGGCTCCC
TCGGAAGAGCTGCTCTTAACAGTTCGTGTTTGGGAGGCAACTTTCTCTTCTTTGCTCTCTCCTTCGGTCTCACCTCATCTGCTCTGTAGTGTTC
AGAAATTCCCCCAAAGTTCTGGTTCAGTTAGGAACATGTCTTCTTTTCCACCTCAGAGTTTTCCACATTTTAAAAACTTTTTCTTGTCATCCCA
GCATCTTCCTCZGGAGAGGAAGTTGATGACTTAGTCCTGTCGTTCAACCTTTTTGAGGCCAAATAGGGTAAGATCATATTAAGGGGAATAATCC
TGTATTCAGGTTCATCCTTTAGTATATAATGGTAACATTTGGATAGTCTAATATTTTCTGGGGTTTTTTTGTTTGTTTTAATATAATCCTTTGC
TTTCTGCTTACTTGAAAACAGTAATTTATCCAACTCAGCACTCAGAGTCAGGGGAACTGTGGTCTCTTAGCTTTGTTTTCTTCAGTAATTATTT
GTTCCGAGAAGAAAAATAAGTTTACCAAAGAAATATCTTTCTGAAAACTCTCCTGTTAGTAGCATAATCTGAGGCAAGACACTGATATTTTAAC
AAAGAGAACAATACACTAATTTACATAATATACATAGGCAATTGGTAGATATGGCTTTAAAATGACTTCTCATTTGGGGGACCTGATAGPTTAG
TTATGTTAACTGAAATTTTTAGTACACTGTTGATAAAATATTAAGTGTATTCTCATTTTTCTGTTATGTCTCAGGTGTTTGCTGACTACCACTT
TGGAGAGTGTATGCTTATCTGCCATGTCCATAACAGAACGAACTGTGTTCCCTTTCTGATGCATTGGGTACCAATTTTCCGCCTTTGGACAGTT
CAGCACAGTAAGCTTTATCATAGATTAATATCCATAGCTACATAGTGATTTTAAATCCACAATAAATCTAGGGTGAGGGAAAGAAGAAAAGTCT
CTCTTATAAAAATGGTCAAACTGTCTCAGTGTGTCATTACAGTTTTTAGGAATTGTCTGTCCTTACTGTCTTTTTTATATAACATTCAATCAAG
CAAAAACTTTTATTTGTTTTGTTAGCTTACAAAAACCTATACTCCTAATCCCTTAACTGGAATTAAGGGGAAATACTTGGAAAGATCAGTTTAT
CTCTTTATTCTTCCACCTTTATCTGCTTTTGAAAGTTCAAAAGTTTGTTTTCTTGTTGTTTTTATCTTTCTGTTTTAATTATTCATAGTACTTC
TCTTCATATGGACTCAGTACTACTGACAAGAGCCATCCTCTGAGATTACTAAGATGACTTGCATCCCTTGAAGTGTTGGAGCCAAAACATGAAG
AGCTAATTCCTTTATCAGATGGGATTTACAAAGCTAGTAAATCAGCTTATACAGGTTGCCTATTTACTCGAATTTAAACAAGCCATTGTTTGCA
AAATAGCAAGGTGAGAACCAAAAGAAAATGTTTCTTTTTTTCCTCTCTCTTTTTTTTGAGAAAGAGTCTTGCTCTCTCACCCAGCCTAGAGTGC
AGTGGCATGACCTCGGCTCACTGCAACTTCTGCCTCCCAGGTTCAAGTGATTCTTGTGCCTCAGCCACCCGAGTAGCTGGGATTACAGGCGTGT
GCCACCACGCCTGGCTAATTTTTGTATTTTTAGTAGAGACGGGGTTTCATCATGTTGGCCACGCTGGTCTCAAACTCCTGGCCTCAAGTGATCC
CACCCGCCTCAGCCTCCCAAAGTGTTGAGATTACAGGCGTAAGCCACTGTTCCCAGCCTGAGAACATTTCTTCATTGACAGTATTTTCATTTAA
AAATTTAGCTGAACTCCAAATGATGAACATTCAAAAGAAAGCATTGTCTTTGGATGATTTGAAAGCAAGTGGCAGATATCATGACACCTCACCC
CTAAATACTACAGCATGTGTCTCCTATGAACAAGGATATTACTCTGCATAAAACCATAATACTATATCACATTCCAAAAGGCTGACATATAGTA
GTAATAAGCTAATATGCAGTCTCCTGTTCCAAGTTCTTATATTTTGTCCCCTAATCCAGGATCCAGCCAAGGATCACTGATTACACTTGGTTGT
CATATCTCTATAGTCTCCTTTAATCTGCAACAGACCTCCCGTCTCTCCCTCTCTCTCTTTCTCTCTCTTTTTTTATCTTTTAAGAGATGGGGTC
TCACCCTTCGGCCCAGGCTGGAGTACAGTGGCATGATCACAGTTCACTGCAGTCTCAAACTCCTGAGCTCAATCAATAGTCCTTCCATCTCAGC
CACCTCACTAGTTGGGACTACAGGTCCACGCCACCTTTTTGCTTCTTTATGTTGTTAACATTTTTTCAGGAGTGTAGGCCAATCCTCTTCTACA
QTGCTCCACAATCTGTTTTTGTCTTTTCTTCCTGGTTAGTTTCAGCTTAAACATTTTTGATACCCATACCACATAGGTGATAGCACATCTATAT
CACATCACTACATCATATTACCAGACAGAGTAATAGCACTTTTTTTCATTATTGGTGATGCAGAGTTTGGTTATGATGCAGATCATTTGTTAAA
GTGTTATCAGTCAGATTGTTTTACTTGAAAGGTCTTTTTCCTTTTTTAATAATAAGTAATCTGTGGGGTACTTTCCCAATATCCTCTCACCTGG
TGGTTTTCATTTGTTCAGTGCTTTACACCCAGACATCTATTGATGGATGTCAGCATCTCTGTGAATCCCCAGGAAATTTTTTTGTGTGCATATA
TTTTCCCAGGCATACAGTCCAGGACCCTCCAAATATTTAAGAGATCTTTTCCCTCTGTTATCAAGGTGCCTTTTACGTGTTTCTGTGCATCACA
GAAGCATATTATAAGCAGTGGGTTTCTAGAAGCTGGGAACCAATCCCGTTCTTCTGATTTTCCTGATTATCTAGCTCACCTCATGTAGAGCTAG
GTACATAATTTTTGTTGTTTGTTTGAGAACGATTGTTCTGGCCTTTCGCTTATCCTGAGCCAAACATTTGGATCTTTCATGTTTGGTATAAAGC
GTACTACAAACTAGAGCAGACAGACTTAGCCCCTGCTTTTTGGTGTTCTCATTTCAAGATTTTGGGGCACATCAATTCQATTTGTGTCTCTGTT
GAACTGAATAGTGGTCATTTGGAAATGAGTTAAAACACTACAGTATATGTTTTAGTTGTCTAATTATAGTTQTATCAATAAQACATTTTCTCCA
AGTGCAGAAAGACTCTTTGACTCTGTAGACAAGAAGGCTCAGGAGATGAGTTTAGGGGGCATTAAGTTTGTTATTAGCCTAATGTGTAATAGCT
CTAGGACTGATTCCTTCTTGATTTTCCCTGCCCAAACAGGGAGCAGTGTGGAATTCATCATGGCAGTGGTTAGGTGTGTTTTGCTTAGCCTATG
ATTAGCCTGTTGGTTACATTGGCTTGCCTTGCATGAAAAAATGTCTCTCAGGTGACTACCAATAACAATAAGAGCTAACATTTGAATAGTATAC
CTTAAAAATACACATTATACCACTTCAGAAGATTATCTGTGAAAACAGATATTTTTATTCTAATCACATAGGTAAGGAAAAAATCTTAGAGAAG
TTAAGGTAGCCAACATCAAACAGCTTGTAAGGAGCAGAGGAAGAACACCCCCAAATTCCATTACAAATTCCTTGCACTTTTTGTCTTACCATTC
TCAAAGGTAGAGGCATTCTAGGGAATCTAAATATGAACAAAGCATGGACCTTGCTCTGAAGGATCTTTTAAACCTAACAGGCGCATAGTCTTAT
GAATCTCTTACAGACAACAAATCGAGAGAAGAGCTAAGGAAGTACGTAAAAGGGCAAAATGCCCTACAAATACAGCGTCAAGGTTCGTATGACA
GAAGTAGGATTTGAGAGAGGTGGATGTTTTGGAAACAAGAAAGGGAGTGACCAGAAGAATGACTATGCGAAAGCACTGCGTTTGTTGTAGAGGT
GGAATTGGCCTAGTTTCCCTCGAGCATCAGCTGCATCAAACAAAGTAAAATAAAATAATGGCAGCTAGAACTCAAAGATAGATTAGGGCCAGAT
TGTGAAGCGCCTTGAATGCCACATGGAAGAACATTCTGTAGGTATTTTGGGCAGGAGATGAAAAATGACCCTACAGAAGGCCCAAAAATGATGA
TAAATGGTTAAATATGTTTAAGTTACCAATCTATGCTAATCTTTTAAGAAATGTTATCTCACTGTAACTATGAATAGTGTACTGTGTCCATTTT
GCAGATGAAAAATGAAGGCTCAGCAAGATTAAGGAAACTGGAAATGTCAAGAGCTGCACCTAGGACCCAGACTTCGGTCTGTTCCATTCTAAAT
CCTGTTCTGTGTCTCACTCTGATTCATATATTGTCACTTCTTTTATTAGAACTGATAAGGTCTTAATCCTCTGTCAAGAATATGCCTGTTAGAA
AAAGCATCTTTTCAAAGTTGTGAAGQGACTGCTTCTCACCATGTTATGTTAAGGAATGTAATCACTACCCAACTTGACTTGTACTGTTCTCTGT
TTTGAGACCAGAGCTAGATGGGAAATTAAAGAATTGGATGATGTTTTATTTACATGCCTGATGACATCAATACATGCTCATAGCAAATGCCTTT
GTTTTCATTTTCAGTATCTAAGCTTATTGGCCCTAAGTAAATCTTAGGTTACGTAGACCTCAGTTCCCAGGGGCATTCAAGATTCATAAAGAAG
TCATATTTTTCCCAGCTAAAATATTTTTCTTCTTACCAGGTTCTCTACTTAAAACACAATCACTACTCATGAAGCTCCCAACAACAATGAAGAA
AGCCCCACAGCCACTGTTGCTGAGCAGGGAGAGGATATTACCTCCAAAAAAGACAGGGGAGTATTAAAGGTGAGGCCACAAAGCTGAGTGACAT
ACAAGGAAACCTGCTGTCTGGAAAGATTATAGCATCTCTTTCTGAGGGGGGCTGGGGTGGATAATGTTTATTGATGGTTACTAAGTGCCAAACA
CTAGAGCTAGATTCTCATTTTGTCTTCATAACTTCAAGGTTTTTTTTAAGAGTTGAGTCTCGTTATCTTGCCCACGCTCGTCTTCACCTCCTGG
CTTCAAGTGATCTTCCCATCTCAGCTTCCTGAGTAGCTGGGATTTTCTTTTTAAATTAATTAATTAAATTTTTGAGAGAGGGCATCTCATTATG
TTGCTCAGGCAAGTCTTCAAATCCTGCCCTCAACCAATCCTCCCACCTCAGCCTTCTGAGTAGCTGGGATTATAGGCACGAGCCACTGTGCCCA
GCTAGACATTTTAATTTTTAATCCCAGATTATAGATGTTGAAATTGATACTGGGCTAGGTAAATTAACTTGCCTAATATCATATATCTTAGTAA
TGCAGATTTACCTGGGGTTCAGGTCCAAGTTATATCTCACTCTAAAGCGCTATATTTCCCTATTCTCACAGTATACAAATAGGTTACTATTTTA
ATGTATCCTTTTACTACACTAAAGCTAGCTAATGACAATTTTGAAGACTTTTTAAGGAAAGAAAAGCTAACTTGAAAAATTCAAAGGGGTTTTA
TTTTATTTTATTTTATTTTATTTTATTTTATTTTATTTTATTTTGAGACAGAGTCTCCCTCTGTTGCCTGGGCTAGAGTGCAGTGGTATGATCT
CAGCTCACTGCAACCTCCGCCTCCTGGATTCAAGCAATTCTGCCTCAGCCTCCCGAGTAGCTGGGATTACAGGCGCCTGCTACCATGCCTCGCT
AATTTTTGTAGACACAGGGTTTCACCATGTTGGCCAGGCTGGTCTTGAACTCCTGACCTCAGGTGATCCGCCTGCCTTGGCCTCGCAAAGTGTT
GGCATTACAGGCATGAGCCACCGTGCCCGGCCCAAAGGGGTTTATTTTTATATATAAATATTGTTCCTGTGTACAGTGAAGATATCTTTTGCGA
TTTCCTCTTAGAATATATCTTACTTTTGAATTAGCTTTCCTACTTATTAAAGCACTATACAGTCATTCACCATATAATGACATTTCAGTCAGTG
ACAGACTGCACCTTCAGCAGTCGTCCCATAAGATTATAATGGGGCCGAAAAGTTCCTATGGCCTAGTGGTGCCATAGCTGTTGTAGCATGGGTA
GTACAGTGTATTATGTGTTTGTGGTGATGTGGGTGTAAACAAACCTACTGCACTGCAGTCACAGTTATCTATAGTACATAATACTTGATAATAA
ATGTTTATATTACTGGTTTATGTGTTTACTATACTTTCTATCATTATTTTAGAGTGTGCTTCTTATTTTTTAAAAAGTTAACTGTAAAACACCC
TTGGCTGAGTGCAGTGGCTCATGCCTGTAATCCCAGCACCCTGGGAGGCCAAAQCAAGAGGATCACTTGAGGCCAGGAGTTCAAGACCACCCTG
GGCAACATAGTAAGACCCCTATCTCTACAAAAAAAATTAAAAAAATTAGCCAGGCATAGTGGCATGTACCTGTAGTTTCTACTACTCAGGACAC
TGAGGCAGGAGGATCATGTAAGCCTGGCAGTTCCAAGCTGCACTGAACTATCATCATACCACTGCATTCTACCCTCGGTGAGAGTQAGATCCTG
TCTCAAAAACAAATGAACAAACAAAAAACAGCCTTGGGCAGGTCCCTCAGGAGGTATTCAAGAAGAACGCATTGTTATTATAGGAGACAGCAGC
TCCATGCATGTTACTGTCCTGAAGACCTTCCAGTGGGACAAGATGTGTTGGTGGAAGACACTGATACTCATCATCCTCACCCTCTGTAGGCCTA
GGTTTATGTATGTGTTTCTGTCTTAGTTWTAAACAAAAAAAAGWTTAAAAAGTAAAAGAATAAAAAATTTAAAAAACAGAAAAATGCTTATAGA
ATAAGGATATAAAGAAAGAAAATATTTTTGTACAGCTGTACAGTGTTTGTGTTCTAAGTGTCATTACAAGAGTCAAAGCTAAAAAAAAAGTTTA
CACAGTAAAAATGTTATAGTAAGCTAAGATTGATTTATTATTAAAGAAAGAAGAAAAATTTTAAAATAAATGTAGTCTAGCTGTCCAGTGTTTA
TAAAGTCTACGGTAGTGTATGGTAATATCCTAGGCCTTCATGTTCACTCACTGCTCACTCGCTGACTCATCCACAACAACTTCCAGTCCTGCAA
GTTCCATTCATGGTAAGTGTTCTGTATAGGTGTACCATTTTTTTTCTTGTATACCTTTTATTACTGTACCTTTTTCTATGTTGAGATATGCTTA
CATACACAAATACCACTGTTTTACAATTGCCTGCAGTATTCAGTCCAGTAACACAATGTACAGGTTTGTAGCCTTGGAGCAGTAGGCCAAACCA
CCTAACGTGGGTGTGTACAAGTAGGCCAGGCCATCTAGGTTTGTGTAAGTACACTCTGCGATGTTCACATAAGGATGAGATCATCTAAGAACAC
ATTTCTCAGAACATATCCCTGTCCTTAAGCGATGCATGACTGTGTGTGTTTCTTCACTCTATAGTTAATCATTTTCACGTGCATTTCTTTTCTT
TTGTTTCTTACAACAATGTGGTGAGACAATCAGAGCATCTCTTGATTCTTCCATCTTATAAGTGAATAAACTCAATCTGACAAAGGTTAAGTGG
CTTTAGTCACATTAAATAGTTATTTCTCCTACACCATTGTTAACACATGTAAGTTTTGTATAGTTAAGTTTTGTTTTTCCTGAAAAGATTATCT
GATCCATTCTGATATTACCAGGATGCTGAGTTTTAATATCTCTTGTCCCAGCAGTAGCAAGTAAGAATTTTTGTTTTTGTGTAACTTTGCTTAT
GTGAAACCCTTCTGTGCCTGGATCTCTTCTGAATATATGTGAATTGCTATTATCTGCTAAGTCAAATTTTGGGGACTTGCAGCTATAGAATAAG
GGCTTTATGCTATAGATTTACATCCTATTGTCTTTTTTTAGAAGGCTTCTCAACCTTGTAGCTATAGTTACTCATCCTAAATTCCAGGAAGCTT
CTAGAAACAGTGGAGACTTTGCAGGCTTTCCTCAGCATCTCCCTGCCTCTGTTTACGACTTACCCTTTCATCTTTTCAGAGTGCCCATGCCCAG
AGAACATGATCCCCAACTTGCCTCAGACCCTGTTCTGTTCTAGGGAGGAAATCTAATTGCAAGATTTTTAGGCTTATTAAGAAACATTTGAGAT
TTGAAGCCCTTTAAATTATATCATTTATCATTGTCTAGAGCCTCGGATTTCACCATCAGGCTTTTGGTTAAGATTTAAATAGGTCTAGTAACTG
TGCAAGCTAGGGCAATTTAGTTAACCTCTCTGTGATTAAGTTCCTGTTTGTAAAATTAGAATAATAATGCCATCAACCTCATAGAGTTATTAGG
ATTAGGTGAAGCACTTAGCATAGTGCCTGTCCTATAGTAATCTATCTTATTAACTATTACGTATTTTTAAAAAAAGAACCAGTGCTGGTTTGGA
AATTCACTAACTATCTGAGGAAGATAAAAGATGGATTTATATTTTAAAAATGAATACAACCTAGATTGTGTTCTTAAGGCAACTCTAGATTTAG
GTACAAATTCATCTTGCTAGGGAAGCCTGAAGTGATACCATAGTACCACTTGCTGGTGTGGATGGCTCTCTGACTCTCTGGTAGCCATTCATCC
CACATGCCCAGTTACTGTTTATAGTCCTAGAAATGCAGATTCCTTCCACGTGAGGCTTACAGTTGAGCATAGCCTCCCAAATACCACATGCAGT
TCGTGAACATGAGCACTTGTAGAATATTGTGTAATATCTTTTAGATTAATGTTTTGAGACTTTTTTTTTCTTTTGAAGTCCTAGAAGGCCAGTA
GTGTTTCTTTGCCTTTAAAGAATATTTGGGGCTGGGTGCGATAGCTTATACCAGTAATCTCAGCACTTTAGGAGGCTGAGGCGGGAGAATCATT
GGAACTCAGGAGTTCCAAACCAGCCTGGGCAATGTAGCAAGATCTCATCTCTACTAAAAATAAAAAAATTTGCCAGGCGTGGTGGCATGTGCCT
GTAGTCTCTGCTACTCGGGAGGCTGAGGTGGGATGATCACTTGAGCTGAGTGATAGAGGCTACAGTCAGCTATGATTGTACCACCGCATGCACT
CTAGCCTGGGTAAAAGAGCAAGACCCTGTCTCAAAAAATAAAATAAAGATGTTTGGGCTTTTGAGTTAAGAAAGCTGTGGAATTTAAGTTCACA
GTCTTTATGTGATATTTATGTTACGTGGACGGTATACTTGTAAAAAAAGAATTTAGCAGCAAATAATCGAATTATTTAAATAAAACTCAAAGCA
AACTCCTTCCCCAAAAAGATGTTCATCAGATGTTAGCTACCATCCTCTGTTATCTACCAAAAATGATTAATTTGGCATAGATTACAAATTAACA
AATTACACCATCACACACCTGGGTTTGATTTTCTTTTCCCAGAACTGACCAAATACATTAGCAGCTTGCTCTGCCAAGAACAAAATAAATACAC
TGGATTTTACAGTAGTTAAATACTTTACTAGAAAACAAGTTATTCAGTATCATATCAACCAGTGCAATGATACTCATGTTTTTTACAGATAGAC
TATGTTTCAAATAATAGGAAATGTAACCTGTTTTACCAGGAATTGGGCTTTCTGAAAATAAACAAGCAGAATCTAGTCAGGAGCTAATGTTATT
TAGCAAAGGAGGGAAATAAAATTACACCTTTCAAGCTTTGCTTAAGCTTCTTTTTCAATATTTTAAAAGTCCACCATTCTTTCTGGTCTAAGTC
CGTGTCTATATGGACTAGCTCACAAGCCTGTGTTAGAGAGTTTAGTGTCGGGCGCGGTGGCTCATGCTTGTAATCCCAGCACTTTGGGAGGCTG
AGGCGGGTGGATCACGAGCTCAGGAGTTCGAGACCAGCCTCQCCAACATGGTGAAACCCCATCTCTACTATAATACAGTAAATTAGCTGAGCAC
GGAGGCGCACGCCTGTAATCCCAGCTACTCGGGAGGCTGAGGCAGGAGAATTGCTTCAACCTGGGAGGCAGAGGTTGCAGTGAGCCGAGATCGT
GCCATTGCACTCCAGCCTGGGCGACAGGGCAAGACTCCGTCTCAAAAAAAAAAAAAAAAAAAAAAAAGAGTTTACTGTCAAAATATAGGGCATT
AAACCAATGAACAGCATTTATTGGTTAATTCTTTGAAGGAATAAATAGTGCGATTAGCAATAAAAGTAGATATAACATAAAAGACAAGTTGGTA
AAGGACAGTTTTCTAACAATGAGTTATTGTGAGGAATGAAGGGAGGCATTAGAAAGAATGGATAATAGTAPATTTGCATATCTGCCTGGATGTC
TTGGGATGGACTGTGTGACCTTTTTATTCTCTGATTACAAGTAAAAAGTTTGACCTCCTTTTTCTTAATGTTTGTCTTTTCTAGATTGTCAAAA
GAGTGGGGAATGGTGAGGAAACGCCGATGATTGGAGACAAAGTTTATGTCCATTACAAAGGAAAATTGTCAAATGGAAAGAAGTTTGATTCCAG
TCATGATAGAAATGAACCATTTGTCTTTAGTCTTGGCAAAGGTAAGAGGCTATTTTTTTGACTTGTGTTTTACTTAACTTTATTACTATTTGTA
ATATATAGGAGCAGAGGATTCTATCCTTTCTAATAAGTAATGAAATTTTAGAGTAGTAGTACTCAAAGAATGGTCTGACAATCCTAGGGCTCCC
TGAGATCTTTTTAGGCGGTCCCAAATGTCATTTATTTTCATAACAATACTAACATATTATTTCCCATTTTACTGTATTGACATTTATTTCCACT
GATGGTACAAAAGCTGCAGTTGTAAAAAAACTGCTGGTTCCTTAACACAAATCAAGGCAGTGGTAACCAGCTGTTCCAGTAGTCATCGTATTTT
CCACTCCCACTCCTTCACTATTTACATAAGGGAAAAAGCTAGTTTCACGTAAAAATGTCCTGATAAAGCAGTAAAAATTATTAGTTTTATTGAA
ACCCAATCTTTGAGTTTCACATCACTTTGATATTCTGTGACAGAAAGGGAAGCACGTGTAGACCACTTCTGTCCCCCAAAGTATGATGGTTGTC
TCAAGGAAAAGTACTTGTGCTCTTGTTTTAGTTGACATCTGAACTAACTGCATTTATTTCATGAGATACCATTTTTACTTGAAAGGGCAAATCT
GATTATGCAGACTTGATTCAGTATTTGGCAGGTGTTTTTCTGACAATGAATGGAGTAAGTCTGTCATTTCAAGGAAAAACAACTGACAATGTTT
TTTGCCATTAAGTTTTCAAGTAAAAATTATAGTTTAGGAGAACTCCTATTTGCCACTGTGGGCTTATTTGCCACTGTGGGCTTGACAGCTTCCG
AAGACTTTTCTGATGAGATTGGTGGGTATTAATGAATGTGGTTTTAAATATACAATGAAATGTGTCCACATTTGGAAGATCTGTATAACTAAGA
ATGAATATTTTCTCAGTGACCAATGCAACATCTTACAAGATCATGTGTGAATAAACAATTCATTCAAAGTTACACCAACAGATTTTAATCTAAC
AGTATGAAAAGTTCACTGATACACTTTCAGTTTCCACATTCTAATTGACTTTTATGAATCTTACAAGTTCTACAGTTTTGATGTAGTATCAGAT
AATTATATTCACAATTATCTAAAAGGTTGTTAAAGGGCTCCTACCCTTTTCAACTATCTATCTTTGTGGGTTTGGATTTTCTTCTCTATGTACT
TCAACCAAAATTGCAACACATCAATGCAGAAATAGGAGAATCTGGTTGTCTCGTGTTACAACTCAAACACATTTCCACATGTATAACATTCTAA
AACAATGACACTCGTCTCACTAATTTTTTTTTTATTTTGGAAAATACAAAATATACAAATACTTTGTACAAAATTAAATTACTTATATTAACAT
GGGGTTATTCTAAAATGAATTATTAAATAACTACTTTTTTACTAAATCTCAGTAGATATGTCAAATAAACAACTTCTTTGGAGTCCTCAATTTT
TAATATGTTAAGGGGTCCTGAGGAAAAAAGGTTGAGAACTACTATTTTCAAGTATTAAGTATGATTTTTACTAAATTATTTTCAAATGCCTTTT
TTTTTTGAGACAGGGTCTCGATCTGTCACCCCQGCCGCCTCACTGTAGCCTCAACCTCTCAGGCTCAAGTGATCCTCCCAACTCAGCCCCGCAA
ATAGCTGGCATTACAGGTGCGTGCCACCATGTCTGGCTCGCTCTGTTGCCCAGGCTGGAGTCTAGTAGCACCATCTCGGCCCACTGCAGCCTCC
ACTTCCGAGGTTCAAGCAAGATTATCATGCCTAAGCCCCCCAACTAGCTGGGACTACAGGCGTACACCACCACACCTGGCTAATTTTTGTGTTT
TTTGTAGACATCCGGCTTTGCCATGTTGGCCAGGCTAGCCATGAACTCCTCACCTCAACTGATCCGCCCACCTCAGCCCACCCAAACACCTGGG
ATTACAGGCATGAGCCACCATGCCCAGCCWAATTTTTTTTTTTTTTTTTTTTTGTAGAGACAGGGTTTCACCATGTTGCCCAGGCTAGTCTTGA
ACTCCTGGGCTCAAGCAATCTCCCCGCCTCACCCTCCCAAAGTGCTAGGATTATAGGCATGAGCCACCACACCTGGCCTCAAATGCTGTTAAAT
GTCTTTAGTTCATTTAATGTTCTTATTTTTTAATCTTGTAGGAATAATAGCCCATACATCAAATTAAACTTTGTAGAACATGTGGCTTATTGGA
AGTTGGTGCCTGAGTTACATCTTTTATTCTTGTCTGCCACAGACAAGCAACTGTAGTTTGTTCCTAGATTAGGGAAAAAAGTATTCCCCCTATT
CACCACCATGGCTATGTGTGGACTTCATTCCCTTGTCCTTCAGCTCTGTATCTAGGTGAATTGCTAACTAAGCATGGATCTGGAAAGGAAATAT
AGTAAGCAGAAACTGAGGAGTTACTTTCTGTCTCTTGCCACTCAGCCTTCCCTGCTTCCTGTATCATTTCTGTGCCTGGTGAATTCTGCTTCCC
GTCCACCTCTGCTCTTTCCAAGCTCCAGTTCTGGCCACGCCCCTGCTCTCTTCTCCTTTGGGTCTTTAGTAGGTTAATGGCTCATGAGATATTA
ATTTTTTGTTTGAATAAGATAGCAGTAAAAATCAAATACAGTCTTGTGGCAGATAATTATGACAGTTATTTTAGAATTTTGTTGTTGTTGTTGT
TCTTCATTTAGCCATATGAACCTCCAGGGCTGCCAGAATGGGTGTCGCATCATTATTTAGGTGACCTAAGAGTGACTGACCTTGTCAGACCCCC
TTGTTCCTGCCACCTGCTAAATCCTTACTCTATCCTTAGAATTCCTGTCTTAAACAAGCTGAGAGTTAAGATAACAGGTCCTTTTAGCAGTTAT
CTTGGTGAGCAGAATTAAGAATTGTTGAGAATTTATATTGGTATCTTCTCTACTGAGATTTTCTCAGTAAAACACTATGGTGAATGAGTTTCCT
GGAATTTTAAGCAGAGTAAGAAGATTTTCTGAGTTATTTTTTTAAAATACAAGTTGGGAGAAAAAAAGAGGCAACTTTCAGTTGACTTTGTACC
TCTGACACACCTGTCTACAGTCAGTGCGGTCTCATGGAAAAAGCTTCCACCCCGCCATTAGGAACCCACGGTTCCTGTCTTCCCTGTTCCTTGT
CCTGTGTTAACATGAGTAAGCCACTTAATCTTACCTGCTTCTGTAAAAAGAAGTCTGTGGAGAGCTTATGTCATTCCATTCTTGTTCCTTCTGT
AAAGAATGCGGGAACAAAAACCATAAAAGACTAATACCAACACAAAGAGACCCTTAGAGGCCTCCTTAAGATGGTAATTGGAAATACTAGGATA
GAGCAATATACCTCTGGTAAAACATCTATAACATAACGTATTTAGTTGCCTACAAAACAAGAAACATTGGTTAGGCAGTTTATAGTTTTGTGGG
GTTTTWTTTTTCCTTTTTTTTTTTTTTTTTGAGATGGAATCTCACTCTGTAGCCCAAGCTGAAGTGCAGTGGCGTGATCTTGGCTCACTGCAAC
GTCTGCCTCTGGGGCTCAAGTGATTCTCGTCCCTCACCCTCCCCAGTATCTGGGACTTCAGGCGCGCCCCACCACGCTTGGCTAATTTTTTGTG
TTTTAGTAGAGACAGGGTTTCACCATGTTGCCCAGGGTGGTCTCGAACTTTTGAGCTCAGGTGATCCGCCTGCCTTATCCTCCCAAAGTGCTGG
GATTACAGGCATGAGCCACTGTGCCCAGACTATAGTTTTATACTTATATTTTATAGCATTAGGTAGTTTATCAAAATGAAGAAAATAATATAAA
TGGACAGTAGTATAAATATTATTCATTGAACTCCATTGATCCCTTATATTGTACCTCTCCCAAGGGAATAAATAGAAATATTTCAATGGAAAGT
CATCCTAATTTGGGGATTCTAGTATGACTTCAGTATTTAACATAGAACTATAAGACCCTATCTGTAAAAATCCCATTGAAAAGATAAACATATC
ATAAAATACACAAATTCAAGCTATACCTACACTTGCTTTGTATATGTCTTTTGCTTTGTGTATGTCTGTTGCTTTTATCATACCATCTTTTTCT
CTTTTCTTTTTTTTTTTTTCTTTCTGAGACGGAGTTTTACTCTTGTTGCCCAAGCTGGAGTGCAATGGCATGATCTCAGCTCACTGTGACCTCC
ACCTCCCAGGTTCAACCAATTCTCCTGCCTCAGCCTCCCAAGTAGCTGGGATTACAGGCATGCGCCACCACACCTGGCTAATTTTGTATTTTTA
GTAGAGACGGACTTTCACCATGTTGGCCAGGCTGGTTTTGAACTCCTGACCTCAGGTGATCCACCCGTTTTCGCCTCCCAACGTGCTGGGATTA
CAGGCATGAGCTACCGCGCCTGGCTAGCCGTACCATCTTTTAAACTCTATTCTTTACTTTCTTCATCTAAGGTTATGATAATCATATATACTAC
ATATATTATATATGATTATATAAGTATATATTATTATGTTGAGGATTAAGATAATGTACACCATCGCCTAGCATTCAGCCTAACAAATGGTAAG
CACTCAGATGTTGATTATTATTGTGGCAGTGTTAGGATTTTTCACTGGTTTTATAATGGAATCGTAAAAGCTGTGTTATAACATCTTGGGTCCT
GACTTCATCTGTTGACTATGCATGCCTACAAACCTTGGAAACAATGCTCAGCCATCTTGCTTAGCCCCAGGAAGTTGGGAAAGACAGAATCAAT
CTAAGGGAACTGATTATTACTACTGTGGAATATGTThCAGAAATTAGAGTGAAGAGGTGATGATTTCTCTTACTAGGTTTTCTTAACTAAATAT
TAGAGGTTGTGAATTATTAGAACTGGTCATAGCCCATTTAAAAAGAAGCTGAATTAAGAACAAGGATTTTCATACATACTTCTATAATAGGGGT
TCTGTTCTTTTACCCTACTCCCCCCAGCAAATATATTTTCACTAAAATGGAAGAGGGCAATAGATTTGGAGGATGAAACAATTATTTCTACCCT
TTTTTTTTTTTTTTTTTTTTGAGACAGAGTCTTGCTGTGTCGCCCAGGCTGGAGTGCAGTGGCGTGATCTCCGCTCATTGCAACCTCCGCCTCC
CGGATTCAAGCGATTCTCTTGCCTCAGCCTCCTGAGTAGCTGGGACTACAGGTGCATACTACCATGCCTGGCTAATTTTTGTATTTTTAGTAGA
GATGGGGTTTCACCATGTTGGCCAGGATGGTCTTGATCTCCTGACCTCGTGATCCACCCACCTCGGCCTCCCAAAGTGCTGAGATTACAGGCGT
GAGCCACCACACCCAGCCTCAATCTTACTATTTTTTGGATTCTTTGTACAATTAGTATAATTTAGGTGACAATCATATTAATATTTGTGTCGAA
QCCACTTTGGTTTTTTTATTGAGGTATTATTTACGTATAATAAAQTTTACCCTTTTAAGTATATAGTTTGATGAATTTTGATAATATTTCTGTA
ACTACCACCACAATCAAAATATAAAACAATTTCTTTACCCTAAAAAGTATTTTTATGTCCTTTTGCAGTCACTTTCCTTCCTGGACCCTGAGCC
CCAGGCAGTCACTAACTTCGTTTCTGTTACTATAATTTTCCCTTTTGTACAATTTCATATAAACAGAATCTTGTATTATCTTTGGTGTTGACTT
CTTTCACTTAACATAATGACTTTGGGATTCATGCATGTTGAATTCCTGAGAAGTATTCCACAATTTGGCAATCCCACAGTTTGTTCACCCATTC
AAAAATAGGACATTTGGTTGTTTGCAATTTGGGGCTATTCAGTTTTTCATATCCTTATTAACACGTAGTATTGTCGATCTTTTTAACTTTAGCC
ATTCTAGTAAGTATCAAGTAGTATCTCACCTTGGTTTTTGTTTGCATTTCCCTGATGACTAGTGATGATTGAGGGTCTCTCTTCTTGTGCCTAG
TGGGCATTTGTATGTTTTCTTTTGTAAAGTGTCTGTTCAGATCTTCTGCCCATTTTAAAACATCAGTTGTTTGTCTTACTGAGTTGTACTTAAA
AGGACAAGCACTTTGGCAAATATGATCTCCCAGTCTATCTTGCCTTTTTATTTTCTTAATTGGTCTTCGGAAAAGTAAAAGTTTTTACTTCTGA
TTAAGTCAGCTTTATCAGTTTTTTCTTTTATGGTTTGGTTACCTACTGTTATTAACCATTTTTGGTTTGATGTGATTTTTTGTTTACTATATTA
CAGACTAAGGTACAGAGAAGCTCGGTAAATTGCCCAAGGCTATACAGTAAGTGGCAGAGCCAGGATTCAATTCCAGCTAGTCTTTCTTAACTTA
TGTTAGATCTTAAGGTTTAAAACATAGCCCAAACCAGGTACAGAAATGAAGTAGGTCCTGGTTAGCAGTATTTATCGAGTGCCTGATATACAGT
AGAAATTTCTCTTTTTTTTTGAGACGGAGTCTCGCTCTCACTCAGGCTGGAGTGCAGTGGCACGATCTCGGCTCACTGCAACCTCTGCCTCCCG
GGTTCAAGTGATTCTCCTGCCTCAGCCTCCTCACTACCTGGGATTACAGGCATGCACCACCATGCCTGGCTAATTTTTATATTTTTAGTAGACA
CGGQGTTTCACCATGTTGGCGAGGATGGTCTCAATCTCCAGACCTCQTGATCCGCCTGCCTTGGCCCCCAAAGTGCTGGTATTACAGGCCTGAG
CCACTGCACCCAGCCACAGTAGACATTTCTTACCAGTAATGATATGTCATACTAAGGTTTATCAAAAGCACTGGCGCTTGTGTTCGCTCGCTCT
CTCTCTCTCTCTCTCTCTTTCTTCCTCCPCCTCCCTCTTTCTCTCTCTCTTTCTCTCCAGAGACTTGCTCTGTTATTGGGGCTGGAATGCAATG
GCACCATCATAACTCACTACAGCATTGAACTCCTAGGCTTCAGTGATCCTCCTGCCTCAGCCTCCCAGAGTGCTCGGATTACAAGCATGAGTCA
CTGCACTCAGCCAATATATTCTTAGTATCTGTTTACTTTTTTTTACATGCAGTTTAGGAGCATTCAGGAGATTTTTAAGGAAAGCTTTTAGGCT
AAAATATTTAATTTTTTTAATTGTCCCCAAAAGTGTGAGCCAGAAAGACAGCAAACTTTAAGAAAGTACTTACTTTGGTTTTTCCTGGTACTTT
GCGATAGAGGAGGAATGCTTCCAATTTCCACTGCTGAGTGGATATCAGGGAGGAGCTGATTTAGGCTGTGGAAAGCTGAGCAAATTTCTCACTC
CATTTAAATTCGATATTACATCATGCCTTTGTTCTCTTTTTGAGTACAGTGTTGTGGTGCTATAGTTCTTTGCAAAGAGTAATTAAAACATGAC
CCCTACCTTCAAGTATGTAACAATTTAGTCTGGCAGAGTTGATCCTTAAGGCTGTCCCAGGACAGATAATCCTACCATATCTGTTTTCTTTCCC
TGTTTGTTTGTTTGTTTGTTTTAATTATAAGCCATGAACGTCGGGAGCGATTATCCTGTGATTAGTTCTCTTAGTAGTTAGAGAAATGACAAAA
ATACAATGTAACATAAGATATGAACACTTTAAAAGAGTCTTAAACATAAATTACTGTAATCTCAGCAGTTTGGGAGGCCAAGGCAGGAGGATGA
GTTGAACCCAAGCGTTCAAGACCAGCCTGCACAACATGGTGAAACCCCATCTCTACAAAAAATACAAAAATTACCCACGCCTGCTGCTGCACGC
CTATAGTCCCAGCTTTCTCACGAGGCTGAGATCGGAGGAPCACTGGAGCCCAGGAGGTCCAGGCTGCAGTGAGCTATGATCACATGCCTTCACT
CCAGCCTGGGTGACAGAGCGAGACACTGACTCAAAACAAAATACATAAATTAATTTGTTTAATTCATGATTAGTTACTATTGGTAGGGAGTAGG
GATGATTATTAGAGCCCTTTTATAGCTATTTTAAAATAAAACTAGGCCAGGCGTGGTGGCTCATGCCTGTAATCCCGGCACTTTGGGAGGCCGA
GGCGGGCAGATCACCTGAGTTTAGAAGTTCAAGACCACCCTGGCCAACATAGTGAAACCCTGTCTCTACTACAAATACAAAAATTAGCTGGTGT
GGTGGCACATGCCTATAATCCCAGCTACTGQGGAGGCTGAGGCAGGAGAATCACTTGAGCCTGGGAGTGGAGGTTGCAGTAAGCCGAGATTGTG
CCATTGCACTCCAGCCTGGGCGACACAGCAAGATTCCATCTCAAAAAAACAAAAACAAAAAAACCTAAAATATATGAGGAAAAGGTATACAGGA
AAAATAATTTGCTTTATTTAGTATAAAAGAAGAAAGGATTTCTGTATTATCTGTTATATGAGGTTTTTCTTATTTTTAACATAAAAAATTAGCA
GAATATGCAAGTAGTTAAAATAGAGAWCAGAAATTGGTACTGAGTCCTAGGAATCCCTCAAGGAAGGTGAAGCAGGAATGAATCAGATGCATTT
TTATTACCTGAAGCTTATTAGTGATTAACCTTGTTTGTCAGCTTTTATATTTTGAAAGCAAATCTTTTACAGTTAAAATATTTACCTTTCTTGT
TCTTTTCTACTCCCATATGAAAAAGTAATTTTTTTTCTGTATGATATGGTGTAGCTTTCATCCTATAGTGTGTTCAAGATGCTCATATTCAAAT
GTAAATGTACATTTAAAAGGTAGTGATTTTTCAACAAGGGTGCCAAGACCATTCAGTGGGAGAAAGAACAGTCTTTTCAACAAATCGTGTTGGA
AAAGCTAGATATCCACATGTAAAAGAATGAAATTGGGTCCTTATCTTACACCATCTGCAAAAATTAACTCAAAATGGATCAAAGACCTAAATGC
AAGATCTAAACCTTGCATTAGAAACCTTCCCATAAGAATATGGAGGAAGGCCGGGCACAGTCGCTCACACCTCTAATGCCAGCACTTTCGCAGC
CCGAGGCGGGCGGATCACCTGAGGTTAGGAATTCAAGACCAGCCCTGACCAATATGGTGAAACCCCGTCTCTACTAAAAAAAAAAAAAAAAAAA
AAAATTAGCTGGGCATGCTGGCGCATGCCTATAATCCCAGCTACTTGGGAGGTGGAGGCAGCAGAATCGCTTGAACCCGGGAGGCAGAAGTTGC
AGTGAGCCAACATCACACCATTGCACTCCAGCCTAGGCAACAAGAACGAAACTATGTCTCAAAAAAAGAAAAAAGAAAAGATAAAGAAATATGG
AGGAAAGGCTTCATGACATTGGATTTTGCACTGGTTTCTTGGCATGACATCAAAAGTACAGGCAACAAAAGTAAAACAATTAAACATAAATTAC
TGTACATCATAAATTAAACATAAATTAGAATACATCAAAATTAAAAACTTTTGTGCATCAAACAGTAACAGAATGAAAGGCAGCCTATGGAATG
GGAGAAAAATGTTTGCAAATCTTATATCTGATAAGGCATTAATACCCAGAATACACAAACAACTCCTATAACTCAACAAAAACAACAAATTAGC
CCAATTTAAAAATGGGTAAAGGACCAGTTGCAGTGGCCATACCTGTAATCCCAGCATTTTGGGAGACCTAGGTGGGAGGGTCACTTAGACGCCA
GGAGTTCAAGACCAGTCTGCGCAACATAGCGATACCCTGTCTCTAAAAACTTTTTAAAAACTCAGGCAAAGGGCTTGAAATTTCTCCAAGAAGA
TATAAATGGACAJCAAGCATATGAAAGATGTTCAACATCACTAATCATTAGGGAAATGCAAGTCATTAAACCACAGTGAAGTATTACCCTCAAT
ACCGGTTAGAATGGGTACTGTTAAAAAAAGAGAAAACAACAAGTGTTGATGAGGATGTAGAGAAATTGGAACCCTTATGCGCTGTTGGTGGGAA
TGTAAAATAGTGCAGCTGCTGTCGAAAACGGTATGGAAGTTCCTCAAAAAATTAAAAATAGAGTTACCATGTTGATCTAGCAGTTCTACTTGTG
GGCATATAACCAACAGAATTGAAAGCAGGGTCTTGAAGAGATATTTGTACACTCATGTTTGTAGCAGCATTATTAACAGCAACCAAAAGGTGGA
AGCAACTCAAGTGTCCTTCAATAGATGAATAGATAAACAAAATCTGATATAAACGTACAGTGGAATATTTACGTTTTAAAGAAAAATTCTGGCA
GATGCTACAACATGQACGAACCTTGAGGGCATTATACTAAGTGAAGTAATCCACTCACAAAAAGAAAAGTACTGTCTGATTCCACTTATATGAG
TTATCTATCCAGAGTAGTCAATTCATAAAAACAAAGTAGAATAGAAGTTGCCAAAGATTAGGGAAGGAGGAAATGCTTAATGGGTATAAAATTT
TTAGTTTTCCAAGATGAAAAAGTTCTGGAGATTGCTTCCACAACAATGTGAATATACTTAAAAATAGTGAACTGTGCACTTTAAAATAGCTAAG
ATGGTAAATGCTTTTTTTTATCCCCCCACAATTAAGCGTTTTTTTTAAAACTCAAGGACAAAACATTATGTAGTCAGAATGTTCTGAAGTGGTC
ACTATAGTCTTTACTATAAATGAAACTGTTAAGTCATTAAAATGTTGTATCTTAAAATATGTTATATAATTTTTTTAAACCAGAAAAAAGTGTA
ACATTACAGAACCAAGAAAAAGTATAATGCATTGCTTTTATACTGGCCTTTGTATCCTTTTAAACTTTTTCACCTGCCTATAGTTACATACGTA
TAAAAAGCATTTAAATTTAGTAATAGCTATATTATACATTTGTATACATATATATTCTTCTTTTATATTTTATTACAAATGGAATAAGACTCCT
GAAATTCATAAATTGTGTATAAGCTAGTAAAACTGAAGTCCAGATCAAGTGTAAGAATAAATTGACGTGGCCAGGCACGGTGGCTCACGCCTGT
AATCCCAGCACTTTGGGACGCTGAGGCAGGCGGATCACGAGGTCAGGAGATCGAGACCATCCTGGCTAACACCATGAAACTCCATCTCTACTAA
AAATACAAAAAATTAGCCAGGAGTGGTGGCTAGCGCCTGTAGTCCCAGCTACTCGGGAGGCTGAGGCAGGAGAATGGCGTGAACCCGGGAGGCG
GAGCTTGCGGTGAGCAGAGATAGCGCCACTGCACCCCAGCCTGGGCGACAGAGCGAGACTCCATCTCAAAAATAAATAAATAAATAAATAAATA
AATTGACATTATGTATTCTTGGTGTGTTTTTTTTTGAGACGGAGTCTTGCTCTGTCGCCCAGGCTGGAGTGCAGTGGCGCAATCTCGGCTCACT
GAAAGCTCCGCCTCCCGGGTTCACGCCATTCTCCTCCCTCAGCCTCCCGAGTAGCTGGGACTACAGGCACTCACCACCACGCCCGGCTAATTTT
TTGTATTTTTAGTAGAGACGGCATTTCACCGTGTTAGCCAGGATGGTCTCAATCCTCTGACCTCGTGATCTGCCCACCTCATCCTCCCAAAGTG
CTGAGATTACAGGCGTGAGCCACTGCACCGGCCTATTCTTGGTGTATGTTACCTATGACTGAGATCCTGAAGATGAAGAATCTTTAAAATGTTT
ATGCTTTGGGGGTTGGGCATGGTGACTCACACCTGTAATCCCAGCACTTCGGGAGGCCGAGGTGGGCAGACCACTTGAGGTCAGGAGTTTGAGA
CCAGCCTGACCAACGTGGTGAAACCCTGTCTCTACCAAAAAATACAAAAATTACCTAAGCGTAGTGGCACGTCCCTGTAATCCCAGCTACTTCG
GACTCTGAGGCACGAGAATTGCTTGAACCCACCACGCAGAGGTTGCAATGAGCCAAGATCGTCCCACTGCACTCCAGCCTGGGCAACAAAGTGA
GACCGTGTCTCAAAAAGAAGAAAATATTTATGTTTTTGGTCCAGTAATTCTACATCWAGGAATTTAACCTAAGGAAATGTCTAGAAGCTTGCTT
AACCCTCTCCAGACTAATTGATGCTCTCCATTCTGTTTGCATATATACTCAGTGATACAACTGGTTTCTTGTTCTGTGGTGTGGTCTCACACAT
TGCTCCCGTCACTGAGTGGGCCTTGTCAGTGCCCTGCAGCCACAGACCACTAGGAACACACCTATAGTTCAACACGTTGGTTCATTACTTGTGA
GGGAGGATGCACACCATGCGAACTGTGGAGTATCTCAGTAAGAGTGTTAGGAGGAATATTTTATAGGACTTGTGCTTGTATTACCTCATTTTGG
GGAGTTTAAGAAAGCAGAGCTTTCTCGATTGGATGCTGTTAGCATGTGCCGTCATTGATGTTTGGGTATCTTAATAAATCTTATTACGACGTGG
GCCAACTACAGTAGAGTCAGACCAAAGCTAGAACTGGTAGAGAAGCAGCAGTCACTCATATTAGCCAGGAGAGGGGGATGTTTGGTATTTTGTG
AAAATGCTCAGTGACCTTGTTTTTTTCCTCTGGGCTTAGACAAAAAAAAATTTTTTTTTCTTTTTTGAAATGGAGCTGGGGTGCAGTGGCGTGA
TCTCAGCTCACTGCAACCTCCAGCTCCCAGGTTCAAGCAATTCTTCTGCCTCAGCCTCCCAAGTACCTGGGATTACAGGCGTGAGCCACCGTGC
CCGGCCAGACAAAATTTTGAAGTATTTTTGTTTTGTCTCACTTGAACACAGTCTCAGAGTGACCTTGTCTCATGTTGATATTTTGCGAGATTGT
TTATGTCCCACAGGAGAATAATATGGCCTCCCTATGATCCCTAGGCCAGCTTCAAACAGCACTGAGGTCCAGCTGTAAGTGTTCAAGCAGTTTC
CAGACGTTGGGGAGGCTTTTTCTTTCTCAAACTCTTGTTAAGAATCAGTTGAAGCCAGGTGCCTGTAGTCTCAGCCACTCAGAAGCTGAGGTGG
GAGGATCACTTGAGCCAGGAGTTTGAGTCCAGCCTGAGCAACATAGCAAGACCCTGTCTCTTAAAAAGGAAAAAAACTGGGCTGATGCAATGGC
TCACACCTQTAATCCCAGCACTCTCGAAAGCTGAAGCAGAAGTATCACTTGAGGCTACGACTTCAAGACCGACCTGGGCAATGTAGCAAGACTC
CATATCTACAGAAAATCATGGTGGTATTCACCTGTAGTCCTAGCTACTAGGGAGGCAGAGGCAGGAGATTCTCTTGAGCCCAGGAGCTCAAGAC
CAGCCTGGGCAACATGGTGAGACTGTCTACAAAAAAAAAAAAAAGAAGAAGAAAAGAAAATTAGCCCAATGTGGTGGTACATGTCTGTGGTCCC
AGCTACTTGGGAGGCTAAGGTGGGAGGACCGCTTGAGCCTGGGAGGTTGAGGCTGCAGTAAGCTATACTCACATCATTGCACTCCAGTCTGTGT
GACAGAGCAAGACCCTGTCTCTTAAAAAAAAAAAAAGTGATGTAGCCATTTCGTAAAGACAGTTGGGCAATAACTATCAAATTTAAAATGCATA
TCACCTTTGCACTTCCAGGAATTTACATTCCAAATATACTTATGCAAAGTAGGCAAACATACATGCACAAGTAGTCTCATTAGATTACTGTTGG
TAATATATGCATCAATAGAAGATTGTAGTATTATACACCCATTAAGAATGAGGTAGCTCTATTTGCATTTATTATGATGATGATGTCTTTCCTT
TATCAAATACATATTATGTGCCGGGCACCGTGCCATGCATTTGATGTGTTTTATCTAATAACTCTTTATTTATTTATTTATTTTTGAGACGGAG
TCTCGCTCTTTCGCCCAGGCTGGAGTGCAGTGGCGCTATCTCGGCTCACCGAAAGCTCCTCCTTCTGGGTTCACGCCATTCTCCTGCCTCAGCC
TCCCGAGTAGCTGCGACTACAGCCGCTAGCCACCACGCCTGGCTAATTTTTTGTATTTTTAGTCGACATCCGGTTTCACCGTGTGAGCCAGGAT
GGTTTCGATCTCCTGACTTTGTGATCCGCCCGCCTCAGCCTCCCAAAGTGCTGGGATTACAGCCGTGAGCCACCACGCCCGGCCTTATCTAATA
ATTCTTAAAACAACTATTAATATATGGCAAGTACTGTTAWTATTTACATTTTGCAGGTGAGGAQACTAACCACAGACATTAAATAACTTGCACA
AGTTTACACAGATAGTAAAGGGCAAACTTGGTATTCAAATCCAGGTATTCTGAAGTTAGAGTATGCTGCATTTTTGACCTCTAAAATGTACATC
CTCCTTTCTACTGATGGAGAAAGAGGATGAAGATATATAATAAATGAAAAAGTAGTTTAGAACTGAATGTCTAGTTTGCTGTCATTTGTATTTT
AAATTTTTTTAAAAAGTGACAGCACACAGTAGGATACCAAAGAAACGTAACAGTTACTTATAAGGAGCAGATGGATGGCCTTTCAGCTTTCACT
TTCCATCCTTTCATGGGTGTTTTAATAATAGGCATGTATTACTTGATTTTTTTTTTTTTTTTAAGAGACAGGGTCTCGCTCTGTCACCTAGTCT
GGAGTGCAGTGACATAATCATTGCTCACTGTAGCCTCTTACTCCTAGGCTCAAGCTCTTCTCCTGGCTTAACCTCCAAAGTAGCTGGAGCTACA
AGCATATGCCACCATGCCGCACTAATTTTTTAAATTTTTGTAGAGATGGAGTTTCACTATGTTGCCCTGGCCGGTCTTAAAACTCTTGCGCTCA
AGTGGATGCTCCTGCTTTGGCCTTCCAAAATGCTGAGATGAGAGGTATGAGCCACTATTCCTGGCCACTTACATATATTTTTAACTGAAATAAA
TGTAGCTGACATTTTGAAAAGTTGTAATATTTAATGAAGATTAAGCTAAAGTCATGAATAAAAAATGGTTTATCTATCCCTGCTTACATACTGT
TATGTAGGGTTTCCCTTGTCCTAATATCCTACCACCAGCTCACTTCAAATTCACTTCCTCTGGGGTTCATCTCTTATCTGATCCTACTTTTGAA
TACTTACATTTTTTGTTTTTGTTTTTTQAGACAGGACAGQGTCTCCACTCTCTGCCCAGGCTGGAGTCCAGTCGCATGATCTCACCTCACTGCA
GCCTTCACCTCCTCCACTCAACTACTCCTCTCTCCTCAGCCTCCTCAGTAGCTGGCAAGCACCACCACCCCTGGCTCATTTTTTAAATATATTT
TTTTGTGGAGATGCCGTTTCACCATCTTGCCTGCCCTCGTCTCAAACTCCTAAGCTCAAGGGATCTGCCCGCCTTGGCCTCCCAAAGTGCTGGG
ATTACAGCTGGGATTACAGGCGCGAGCCAACGTGCCCAGCCCCCAGATACTTAATATTTCTGATTGACAGAGTACCTCATTTGTCATTTAGCCT
TTTTTTTTTTTTTTTTGGAGACAGAGTCTTGCTGTGTCGCCCAGGCTGGAGTGCAGTGGCATGATCTCTACTCACTGCAACCTCCGCCTCCCAC
GTTCAAGCAATTCTCCTGCCTCAGCCTCCTAAGTAGCTGCGATTACAGCCACGCACTACCATGCCTGCCTAATTTTTGTATTTTTAGTAGAGAC
GGGGTTTCACCATGTTGGCCAGGCTTGAATTCCTGGCCTCAAGTGATCCGCCTGTCTTGGCCTCCCAAAGTTCTGGGATTACAGGCGTGAGCCA
CCATCCCCCACCAAAATCTTAATTTAGCCTTTTAAATCGCCTAAATTPTCATTGTTTCCGAAAGGACTTGATACTGCTTACAGTAAGATAATGA
TGCACATCATAGGAACTATAAACATTGAGATTACAATTATATAGCCTAATATAGTTCTACAATTGAGCAGTAAATTTAACTTAGTTTCCTGGAA
GTAGGCAAAAAATAAAATATTTTAAGTTCTGTAGTTGTGGTTTGACAAAAAGACACATATGTTTTTGTTTAAAGGAACATTTTTCTGATTCTAA
ATTTTGAGAAGAAATGTGTTTCATGGACCCTTTTATGCAATTTATCCTGTTCTGGTTACTTCTTGTTTTGTTGTTTTTTTTGTCTACCCAACTA
AATTATAAGCTTCCTTAGGACACACTCTATTTTTCAATACTATTTTTGTTCTATCTCTGCCTAGAAAATTTGCGCTTATTATAGTTGAAATGAT
AAATTACGCAACAATTGCAACATAGCCATATTTCATGGATAATGTTACCACCACTTTTCAAAGACTTAATTTTTGACTACTTTAGGAATTTATA
TTACTTTCATCCCTTTCAGCAGGGAATCCTTTTTGAGACAGGGTCTCACTCTGTCACCCAGGCTAGAGTGCAGTGGCATAATCATGACTCACTG
AAGCCTCAATCTCCTGGGCTCCCAGGCTTAAGCTGTCCTCCCACCACAGCCTCCGGAGTAGCTGGGACTACAGGTGTGCACCATACCCAGCTGT
GTTCTTTGGTTCTTTGGGGTGTTTTTTTTTTTTTTTTAGAGATGAGGTCTTGCTGTGTTGCCCAGGCTGGTGTCAAATTCCTGGACTCAAGCAG
TCCTCCCACCTCAGCCTCCCAAACTGCTGGGCCGATCAACAGGGAATCTTTTGTGATAGCCTTCAAAAAAAATAATTTATTTATTTGTATGTAG
CACTCTCTCATCTTGTAGCCCATACTTTCTAGCTGAGTATGTAGTATGAAAAACTGGAAATTAGAACTTTGGTTTTATGAAAGTTAAATCTTTT
TAAATACGGCTTATACCTTTCAACTACCTTTTCCTTTCCCTTAGGCCAAGTCATCAAGGCATGGGACATTGGGGTGGCTACCATGAAGAAAGCA
GAGATATGCCATTTACTGTGCAAACCAGAATATGCATATGGCTCGGCTGGCACTCTCCCTAAAATTCCCTCGAATGCAACTCTCTTTTTTGAGG
CAAGTATGTGTGTGATGTTGCCTTGTCACCATCTGCTCTACAATAGCAATGGCTTTGAAGCAACAGGCAGTGGGAAAAGGAGCTAAGCTTGCTA
TAGAGAATCAGATCCAACTTACTTATTTTGGAGCATGGCTTCTTGAATTGTTTCCCACAAACCCAAGGCTGCGCGTGCCAGTGCTTGCTTACAA
ACGTGTTTCTAGCACAAAAAGGTCATATCCAAATAAACAAAAGACGCTACACTTAGAAATAAAGATTCTGGTCCATGGTGTGAGTGTGACATAC
TCAAAATTGACCAGAGTACAGGATGGAAATGTTGGCGATAAATGTTGAAATATGCCTATGTCACCTTGCGGCGTATTTACATATTATAAAAATT
TATGTGTCACAGATCTCACTTCTCTAGAAGCCGTTGGTGGTGATAAAACATTATTCATCCTATGTTGAATTACATATATTTCGCAAAACTTGAT
TTTTAAGAACCACCCCAGCTACGTAAAATACACCTTTAATCAGTCGTAGGTCACATCCATGAGATTTATTCTTACATTTTGAGAAGGCAACTCC
ATAAACCCAGGATGGGAAAGTTGTGGCCTCAAACAAAATAGTTCTACACCAGCACCTTGTAGCTCTGGCTTTTTGCCCACAGGGTAGTTTTCCT
AGTATTTCCTATCTTTGCACTGCAGTCCCTTAGGATATAGTTCTAGACATTAGCATTACATGAAATTCTTTCCAAGTATGGAAAAGTATAGTGG
GTTTTTTTTTTTTTGCTACTTTTCCAATAATTAAGATGCATAAATGAATGTATATATTTTAAAACAGCTTTATTTTGTAAAATGAAATAATTGG
TTCCCCTTGCCTTCACATATGTTCAGTTGGCTTTATTTGGTCTTTTCTCAGCTGAATTCCTTTTTTCTTTCTAGATTGAGCTCCTTGATTTCAA
AGGAGAGGATTTATTTGAAGATGGAGGCATTATCCGGAGAACCAAACGGAAAGGAGAGGGATATTCAAATCCAAACGAAGGAGCAACAGTAGAA
AGTAAGTATTAAAGTAAGAGGGAACTAGTCCTTAAAAGTTGGGCCATTTATGTGGACGAAATGGAGACTTAAAAAAAAATGTTTGTAGAAATGA
ATATATCGGCTGTGCGCAGTGACTCACGCCTGTAATCCCACCACTTTGGCAGCTCCAGCCGGQTGGATTGCCTGAGCTCAGGAGTTTGAGACCA
GCCTGGCCAGCATGGCGAAACCCCATCTCTACTAAAATACAAAAAACTAGCCAGCTGTGGTGGTCCACGCCTGTATTCCCAGCTACTTCGCGAG
GCTGAGGCATGAGAATCACTTGAACCCGGGAGGCAGGCGTTGCAGTGAGTCGAGATGGTGCTACTGCACTCCAGCCTGGATGACAAACCAAGAC
TCTGTCTCAAAAAAAAAGTATTGAATATATCTCAAAGATAGTCTTAGCTGGGCACAGTCGCTCATCCCTGTAATTCCAGCACTTTGGGAGGCCG
AGGCAGGAGGATCTCTTGAGCTTAGGAATTCCAGACCAGCCAGCACAGCATAGTGAGGCCCCATCTCTACTAAAAACCATCAAGAGAATTAGTC
AAGTGTGGTGTCACGTCCCTGTAGTCCCAGCTACTTGCGAGGCTGAGGTGGAAGAATCACTTGAGCTCTGGAGATGAATGCTACAGCAGAGAGC
CGTGATTGTGCCATTGTGCTTCAGCCTGGACAACAGCAAGATTCTGTCCAAAAAAAAAATAGTTTCAGGTTCAACATTTATTTAATAATTTTAT
TTAGTAAATATTAATTCAACATCTACGTGTCAGGCATGCTACCAGCCTCTACAAACTCAAAAATGAATACAAAGTAATTCCATCCACAAGAAAT
TCTCAAWCTGTGGACTCTCTGGTGTGCCAACAACTATATTAGTTGCCTGATATGTTTGTTCATTTATTTCTCACAACCTTAAGAGGTAAAATTT
CTTGTCCCCAGATGAGAAAAGTTAATTCAGAGATGTTAAATAAATTGCCCAGCGTTACAGAGTTACTAAACAGAGCAGTAGATTATAATCCCTT
TTAGGACTCAAATACATCTCATCAGCTGTCATCAAGATTAAAAAATAGTTCATCATTTAGATCAATATTGGATAATAATGTTCATTCTATTCCA
TATCAGGAGACCTAGGTTCAAGTCCTGCTTTGGTGTTTATTGAGTAACCTGAAGCTATTCCGTTTCTTCATTTGCTAAGTGGGAGTAGGGGCAG
CTCTGAGGACTGAGATAGCTAACTCTTCTTCCCTCCCAGAGCAAATTATATGTACCACTGTGTATACTACTTGPCACTTTACATTGTAATCAGT
ATTTGCCCATTTCTCCCAGTATACTTTGAGTGCTTGAGGACCAGAATCTTTCTTCATCCTTTTGTGTTCAGCATTTCCTGCAGTGCCTGACACA
CATTTCGTCTTCAGTAAAGGCTTTCTGCATGGATAAGTGCTTGAGAAGTGTCATTAGTGAGCTGAAATCCTTCAAAAGAGAGTTGTGAGAAAAC
AGAGAAATACTTCCATTAAGAACAAAAATCAAATCATTCAACAAGGATGGGAACATGAACTATTATTATAAAAGCTCTGTTTAAAATAATTATT
TCAAAAGCCAGGTGTAATGGTGTGCACCTGTAGTCCCAGCTACTTGGGACGCTGACATAGGAGGATCACTAGAGGCCAGGAGTTCGAGGCTATA
GCGTGCTATGGTTACACCAGTGAATAGCCAGTGCACTCTAGCCTAGGTACCATACTGACACCTTGTCTCCTTAAAAAAAAAAAGTAAAGAGTTC
ATTTAAACAGAAAACAAGAATAGCTGAGTTTCTTTCTTTTAAAGTATATACTATTTTCAACCCATATTTCATTTCTACGAGTCTGGCAAATCAG
TTTAATTCTGTTGTTTTGGGAAGCAGTTACAATATTATTTATCCCAATTGCCATTTGGGATAAGTTTTTGAATACATGAAGAGTAGCTTTAAAT
GTAGAATACCAGGGAAATAGCTCCAGACTAGGAGTTGGGAGATAGGGTTCCAGCCTCAGCCCTGGCCTCAGGCTGGCTTCAACTAGACTTCACC
TCTGGGCGTTAGCTTCCTTCCTATCCAGTGACGCAAGCTTGAACCTCTTQATTTTTAACGTCCTACTAGCCCTAAAACTCCATCATACAATTCT
AAATATTTACAAAAGTTGTAGGAATGATAGAAAATCATCTTCACTATATTAGTAAGCACTTAAGCTTGTCTATAATGTCTATTTTAAATTTTTC
TTATGAAAAATTTCAAATGTATGTACAAAGGTAGAGGTTTACTCCTATCGATACAGGCACACCCTAGATTCAGTAATTTATAATATATATTTTT
AAACTGGAGGGTTTTCTCAACTAAGAGGAAAAACTTTTCCCAATTATACACAATATCACAAAATTACCGCTCTGGAGATGACTTTAGAGCAGAC
TAAATTAAATCAGATCTTGCTTTTGGTAATACTTGCGATGGGAGTCAATTTTTTTTTAACTTTTTGTTTTGAAATAATTTTAGAATTACACAAA
AGTTGCAAAACTAGTATAGCCCGTTCCCTTATATGCTTTACACAGCTTCCTCTAAATGTTAACATCTTACCTAATCACAGTACATTTATCAAAA
TCAGGAAGTTAACATTGATGTAGTACCAGTAACTGTTTTCCAGATCTTATTTGAATTTCACCAGTTACTACACTACTGTCGTTTTCTGGTCTAA
GATCAAACTCAGGATCCAGCATTGTATTTACTTTTTTTTTTTTTTTTTTTTTTTTTGAGATGGACTCTTGCTCTGTCATCCAGGCTAGAGTGCA
GTGATGCTATCGCAGCTCACTGCAACCTCCACCTCCTGGGTTCAAGCAATTCTCCTGTCTCAGCCTCCCAAGTACCTGGGACTATAGGCGCACG
CCACCATGCCCGACTAATTTTTGTATTTTTATTAGAGACAGGGTTTCTAACATGTTGGTCAGGCTGGTCCTCAAGCTCCTGACCTTGGGTGATA
CACCCCCCTCACCCTCCCAAAATGCTGGQATTACAGCCATGAGCCACCGCACCCAGCCTACTCTACTATTTTGAATACAACTGACCGTTTTGTA
GAATATCCCTCAATTTGGGTTTCTGGGATGTTTTTAGATTGAGGTTATGCATTTTTTGAAAATACCACTGAAGTGATGTGCCTTTCATCATTAT
CAAGAGGTATATGATATTGATCTCTCATTACTGGTGATGTTACCTTCGAACACTCGGTTAAGGTGGGAAACCATGTTTCACTACTGCAACGTTA
CTATTTTCCCTTTGTAATTAAAAAGTATTAGGAGTGAAGTACTATCTTGTTTTGTATCACAACTTTACACACTCATTTTGACATTTGTTGATTT
TTGCTTGCAACAGTTATTACTGTGGCATTTGACTAATGGTAAATTTTCTATTGCTGTTATTCTCTCTACATTAATTTGAATCCTACTGTATATT
TATTCAGTTTATTTATATCATTATGGATCTGTGTATATTTATTCCATAACTTATTATTCATTACTATTCTTATTTATTTTGTTGTTCACATTGT
CCCAGATTTGACCATTGGAAACTCCTTTAAGTTTGTTTTTGTGTCCTTTCAATATTATCCCCATTTTTTTTAAGTGCTTCCTTATTTTCTGGTA
CCACAAGACACTCATGGCTCATCTTAAAATTTTCCTGCCCTAGACCTGGAACCAACCATTTCTCCAAAGAGCCCTGATTCCTTTTATTGGAGAA
AGGCATTTAGAAACCAAGATCTGACTGCTACGTTTGCTCCTGCTATGGGATATCATCATTCCTAGACCCACTTAGCAGACAGAGAGATAGAGAA
TATATGGACCTCAACATACATACGTATACATCTATATGTATTTCTACATCAGCCTCTGTGTGTGTGTGTGTGTGTGTGTGTGTGTGTGTAGTTG
TTGTTGTTGTTGTTGTTGTTGTTGTTTGCGACAGAGTCTCACTCTGTCGCCCAGCCTCGAGTACAGTGGTGCAATCTCAGCTCGCTGCAACTTC
CCCCTCCCAGGTTCAAGTGGTTCTCCTGTCTCACTCTCCGGAGTAGCTGGGATTTACAGGTGCCTGCCACCACGCCCAGCTATTTTTTGTATTT
TCAGTAGAGATGGGATTTCACCATGTTGGCCAGGCTGGTCTCGAACTCCTGACCTCGTGATCTGCCTGCCTTGGCCTCCCAAAGTGCTGGAATT
ACAGGAATCAGCCACCGTGCCCGGCCAGCCTGTATGTATATGTTAAAAACCAGGGATACTCACTGGCAGTTGCAGTTCCAGTTCCAATCCAACA
CCACAAGTTTATTTGCCCTTTGCTCATTTGAAACTCCATTCTCCAGAAGTGAGAAAATGCTCTCATTCTCCACAGCATACTCATTGCTCCCATC
CTAGTGTATATGTAAGATGGTTTTAGAATTGCTAACCTAGACCCCTGTGACAAACTGACTTTAGGAAATATTTGTTTATAGTACCTTTGGTGAT
CAGTCTTATAGTGTATATTCAAAACACTTGCTTTCCAACTGACGTAACTTCTTTCTTCCTCACCCCCTTCAATGTGATTATGTTACTCAATTGT
AACAAATTATTCATTTGTTACCCTGTATATTCCATTTTGCGTTCCTGCCATGCTCTGGTTAATTTTAATTATTCGTTTTATTTTGGATATGTGA
AACATTACCATGATTTTAAACTATATAAAAAACGATATACTCAAAAGTGTCATTTCCCCCTCATCCTTATTAACCTGTTCCCACTTCCCCATTA
TTTCCACCCTATTCCAGTCTCATTAGTTATATCATTAGATCCTGGTTTTTCTTCCTGTATTTTATTACGTTTTATGTCATTTTATTTTTTTCTG
TTTCTGTTAACAAAGGGAGCCAGTCGTGGTGGCTCACACCTGTAATCCCAGCACTTTCGGAGGCTGAGGCAGGAGGATGGCTTGACCTCAGGAA
TTTGAGACCATCCTGAGCAACATACAGACCTTGTCTCTGCAAAAAGTTTAACATTTACCCACATGTGATGGCACATGCCTCTACTCCCAGCTAC
CCAGAAGACTCAGCTACCAGGATCTCTTGAGCCCAGGAGTTCAAGGCTGCAGACAGTTGTGATCATGCCACTGTACTCCACCCTGGGCAACAGA
ACAAGGCTCTGTCTCAGTTTGAAAAAAAGAGAGAAAGAACAAAAGAAAGGGCAATGACTAATCTTTTGTCTTTCATCTTGTTAGAATGTACTTT
CCTGGGAAATTTTAGATTAAATGCTTGATTTTACTTTTTCACTCAAAAGACCGTCTTGGTTTTCTGGAACAAATCTATTTCATAGAAATTTATT
GTGTTATACTTTCATTTATCAAGGTTAAAATGTCCTGGTTTTTCCTCCTTTAAACAAGTTAGCTATCTGGGGGAGGAGAAAAAAGATTACCCTA
AAGGTGGTCCTGTTCAAAACCAACATCACCTTGTTCTTTTTGAAATCTTCTCAGTCTTTACTGGTTTGCCTTTTCTTTATAGCATCACCAAATA
CACTTACATGTGAAACCAGATATAAAACCTGGAACTAGGCCAGGTGCGGTGGCTCACGCCTGTAATCCCAGCACTTTCGGAGGCCGACGTCGGC
AGATCACGTGAGGTCAGGAATTTGAGACCAGTCTGGCCAACATGGCAAAACCCCATCTCTACTAAAAATACAAACAATTAGCTGGGAGTGGTTG
TGGGCACCTGTAATCCCAGCTACTTGCGACCGTGAGGCAGGAGAATTGCTTGATCCTCGGAGGTGGAGGTTGCAGTGAGCAGAGATTACGCCAC
TACACTCCAGCCTGGGTGACAGAGGGAAACTCCGTCTCAAAAAAATAAATAAATAAAATAAATAAAACCTGGAACTAAAGCCGGCTATAGAGTG
AACAAGAACAATAGACACTGATCACCCATTACAATATGTTGTTATAGATACGTGATTTACTAAATTTCTGTCATTTCTGTGTAAAGACACAATG
AAGTGGTATGGACGAGCGGACAGATAGACTTTGTAAGTGATGGACTTCATTTTTGAATCTTGGAATTGCTACCTGATACTTTTATGACTTTAGA
CAAGTTACTTAACTACGTGGAGCATTCAGTTTTTTTAATCTGTCAAATGGGAATAGTTTTCACTCCCACAGGTTGTGTGACAGTGATAACGGTT
TGGCTGTGTGCCTACCCAAATCTCGTCTTCAATTGTAGTTCCCACAATCCCCACGTGTCCTGGGAAGCACCCACTGGGAGCTAACTCAATCGGG
GGGGCATTTTCTCCCATGCTGTTCTCATGATAGAGTGAGTTGTCACGAGATCTGATCGTTTTATAAGGGTCTGGCAGGGAAACCCTGCTGGCTC
TTCCCTCTTCTGCCGCCATGTGAAGAAGGGTGTGTTTGCTTCCCCTTCTGCCATGATTGTAAGTTTCCTGAGGCTTCCCCAGCCATGTGAAACT
GTAAGTCAATTAAACCTCTTTTCTTTATAAATTACCCAGTCTCAGGCAGTTCTTCATGGCAGCGTGAGAATCGACTAATACAAACAGTTACAAA
GAAGAGTGTCAGACATTTAGTGGACACTTAATTTCTTCTACCCTATATATTGTGTTTCACTAATTTGTTTTGCTTCCTTTTAATTGATCTAAGG
AAAAAGATCCTTGACTCCTCTTTTTCTTTTTCTGTTGCCCAAGCTGGAGTGCAGTGGGCCAGGCTGGAGTGCAATGGCGCATCTTAGCTCACTG
CAACTGCTGCCTTCCGGGTTCAGGTGATTCTCCCACCTCATCCTCCCAAGTAGCTAGGACTACAGGCACGTGCCACCACACCCAGCTAATTTTT
GTATTTTTAGCAAAGACGGGGTTTCGTCATCTTAGCCAGACTGGTCTTGAACTCCTGACCTCAGGTGATCCACCTACCTTGGCCTCCCTAAGTG
CTGGGATTACACGCGTGAGCCACTGTGCCTGGCTGACTCCTCTTTTTCTTATCTCCTACATGTCATCCATCAGCAAATCCTCTTGGCTCTACCT
ACCATGGTTATTAGAGACCTAAAATTGTCTCTAATAACACTCTTCAAATTAATGATATATCTTAATACTAATCTTATCTAAATCACATTTCTCA
ACGTTCCGGGGAAAGAATAACATTATGGACTCAAAAAGGTTTTTACTGAATTGTAAACTTTTACTTCCTTTTCATAATTTGTCTCTGTGTGGAA
GAGAAAACTTAGGTTAGTTTGATGGGATTTATTCTTTCTCAAGGAAATTTTGATTTTTACTCTTTATATGAGGATCCTAATGGTTACAAATCAG
TTTTTAGAATCTGTCCCCCTACGCTTTTTCTTGTGTCAGTTAAGATGACCATCTTGTAATTTTTATTCAAAGTTTTTCTTTCATTTAAAAAGTT
ACTGTGTTCTAAGGTACTCTAATTGATATGATTATGATQACTGATACTTCACTCTTTAAGTTTAATTGCAGGTCTTGGCTACATTGCTACTTTT
CAGCTTTGCTTTCTTAGAAAATCAAATCTGACTTACATAAGGTTCTAAATAGCTACCAGTTACTCTTCCCCCATCCCCCAGTCTCTCACATCGT
CTGATTTGTCAACTTTAGTGGTTGGAAGAQGCCCATCAAAATCCTCGCTGTATGTGGTTCTCTCGCCTCTCAGTACTTTCAGCCACATTTGAAT
GCAAAGAATGCAGACAGTCAGCAGTATTTTTAAATGTGAAATTTTCAACAGAGTGTTCTACAGGTTCCAGTTGTTGGCGTACCTCCTTTCCCTC
TCCAAATCTCACTGAAATGATAGATGGAGGGAACAAAGATTTTCAAAAGAAGAAGAAGAGATCAATAATAGTCCTGGAAAGGAGAGGAATGTAA
GAGTGGATCAGAAATTATGGAGAATTTCTGGAATATGTAAAGTTGATGAAGTTQAACCAATGGAAACTAGTCTCAGCAAATCCAACCCGAGACA
GGTGTAAAGAGGACTTAAGCAGAGGTACACACTGTTCTTCTCAACAGGCTTAGGCGGAACTCCAAGCTCATGCTCATCTGGTGTGAGGGAGCCC
AACCACGCTATGCACCAATCATCACCGTTGTTATAAAACAAGATGGAACAGCGGGGCCAGTAAACCTCCAGTCCACTAGTTCTCAACTTTTCCT
GAATTTAGAATTACCTAGGGAGCTTTTAAAAATTCTGACACCCAGACCAGTTAAATAAGAATCTCACATCTCAGAAGATGGAACTCCAGGCACA
AGTATTTTTTTTTAAACTCCCAGATTATTCTAGTTTGCAGGCAAGGACTGAAAACTACTAGACTACGGTCTGACTGTGACTTTTTCTCCCAAGC
TAAAACCAGTAAGGCCAGGTTCTAAAATAAAATAGAAAAATAATAAAATAAATTTAAAAATAAATAATAAAACAAAATGTGCCTCAAAAGACTC
CAAGTGTTAGGACTTCGACTAGGGAAAATGAATGTATTTTTTGAAACTTTTTTTTTTTTTTTTTTTTTTTTGAGACGGAGTCTTGCTCTGTCAC
TCAGGCTATAGTGCAGTGGCGCGGTCTTGGCTCACTGCAAGCTCCGCCTCTCGGGTTCACGCCATTCTCCTCCCTCAGCCTCCTGAGTAGCTGG
GACTACAGGCACCTCCCACCACCCCCACCTGATTTTTTTTGTATTTTTAGTAGAGACGGGGTTTCACCGTGTTAGCCACGATGGTCTCGATCTC
CTGACCTCGTGATCCACCTGCCTCAGCCTCCCAAAGTGCTGAGATTATACGTGTCAACCACCACGCCCGGCCTTTTTTGAACTTTTAAAACAAA
TTTAAAGGGGAAAAAAGTGTAAAGACAGCTTTTCACAGTAAATAATACTAAAGTGTTCTGCTCCTAAAGTCAACTTTCCTGTGCCCATCATGCC
CAGCTCATAGTAAATGCTTAATAAATATTTGTGAAATCTTCACCCTTAGATCCCCGCAATCAGCCCATCCTTTTGCAGAATGGCCTGCCATGGA
AACATATCCAACTGCTTAGAAAAATGGTGCCAGCTAACTATAGTCATTTGATCATCCAGAATCCTTCCTTTATAATACGGCCAGACAGCAAAGA
ATCATCAGCAAATATCACGAAAAAGCAGCACTAAAATGGACTAACAGAAGAAGTAGAJ4GAGAACCTTATAAATACTAATTATTGTCTTCAGAA
ATTGTTAACAGCACATCAGATTCATAAGTCAAAAAGCCAACTTCCCTGAAAAAGGAAAGGGAGGACAAGAAAGTTTCTGGAAGTTAGAGCATCA
TTAATCAAAACTGAAACTCAGAGGAAGCCTTGAAGACCAAAATGGACAGTCCCTGAAATCAGGTGATCTGAAAGATAGCCAAGGATTTCTCAGA
ACACAAGAAAAAAGATGGATGAAAAGTGAAAAAGAAATTAGAAGATATGGATAATAAATCCAGCAGACGTAATATGCATCCAATCAAACATCTA
ATAGAAAATTTCCTACACGGAATAAAAGAAAAAGATTCTTAAGATCGAAAGGGCCAGTTGATAGTGGTACCCAGGAGGAAGGAGTGGTTTTTCA
CCCAGATATATCCTGGTGAAATTTCTGAATTCTGCAGCTTACAAGAAAATTCTGAACTCTTCCAGGGAAGAACAAGCTATGTACAAAGGAATAA
GAATCTTATTCATTACACTAAAGACAATGCTTTTGATCATTGTCTATACCTATGTGTATTTTGAACCTAGAATTCCGTACTCAGCCCAACTGTC
ATTTATGTATGAGAACAAAATTAACTTTTTGGAATTTGCAGACTCATATTCTTTTCGATTAAAATACTGTGGGATGTACCAAAACAAAAAAATA
AGTCAAGAAGCAACAACTCAAQQCATAAGAAACAGTAGCGAGAAAAAAAAAATAATTTCCATATTGCATACATTTATCCTQAAAGATCTCTGAA
GCAGTACAGCTGTAATGTTAGAAGTAATATTAATAATGAAACAGTCTGGCCTGCCCACACTGQCCCACACCTGTAATCCCTACACTTTGGGAGG
GCGTCACTTGAGGCCAGGACTTCAAGACCAGCCTGGCCAACATGGCAAAACCCCATCTCTAGTAAAAATACAAGATTTAGCCAGGCATGATGGC
ACATGCCTATAATCCCAGCTACTCAAGAGGCTGAGGCAGGAGAATCACTTCAACCCAGGAGACAGAGGTGGCAGTGAGCCAAGATTGTACCACT
GCCTAGGTGACAGAGGGAGACTCTGTGTCAAAATAATAATAATAATAATAAAACAATAATAACAGTCTGGATTCTGGGTTATTTTAACAATAGG
TGGTGAGGATAGGAAATGAAGAGGGCAAGCCAAAATTGTCTAAGATTATTTTCTTTTTTAAAACATTTTTTGGAGATCAGCTCTTGCTCTGTTG
CCCAGGCTGGCATCCACTGGCACAGTTGTCGCTCACTGCAGCCTCAAATTCCTGCCCTCAAGCCATCCTCCCACCTCAGCCACCGGAGTAGCTG
GAACTGCAGGTGCACACCCAGCTAACTTTTTTTATCCTTTTAGAGACGGGGTCTTCCTATATTGCTCAACCTGGTCTTGAACTCCAGGGCATAA
GCGATCAGCCTGCTTCAGCCTCCCAAGTAGTTGACATTACAGGCTCGTGTACTACCACAACCAGCTGCCTTAATTTCTTGTTCCAGGGGAGATT
ATGCTGATTAATTCTTTTATTTTAAGATTCACCGGCGACATGTGCCAGTTTGCTACATGGATATATTGTCTGTAATGCCGAGGTTTGGGCTTCT
ATTGAACCCATCATCCAGATAGTGAACATAGAATATAGCCCTTGTACCCCTCCATCCTTTTTTGCTTTTGGAGTCCCCAGTGTCTCTTGTCCTC
ATCTTTATGTCTGTGTATACTCAGGGTTTAGCTCCTACTTATAAGTGAGAACATGCAGTATTTGGTTTTCTGTTTCTGTGTTAATTCACTTAGG
TTACTGGCCTCCAGCTGCATCTCTCTTGCTGTAGGGGACATCCTTTCATTCTTTTTTATGGCTGCATAGTATTTCATGGTATATATGTACCACA
TTTTCTTTTTTTTTTTTTATTTCAAGTCTGTCAACCTATTCTTTATTTCGGAGAGAGGGCAGGGCAGAGTGCTCAGTCCTTGGCAGCGGCTTTT
GTCATGGTGGCCAGGACATTGCTCAGCTCCTCCTGCTTCCTCTTGGTGCATCCCCACCCTTGTCTTGATGAACTTGAGGGCCCATTTGTCCTTG
GAGACCTTCAQTAACTCCATGGTGCACCGCTCGTATGGGGTGAAGCCACACACCTCTCAGATCATCTCCGGCACGAACTTGGTATATTTGGTCA
GGTGCCTGCAGCAGTGGCTGTGCCTGGGCGTCCTCATGTTCTCGGTCACCTTGTGGCCCTTGTTGAGGCCCATGGCCATAGGGCAGCACAGAGC
CATGGCCGCTGCTCTCCAATGGTGGCCGTGGCAGAAGGAAGCCCACATTTTTATCCAATCCACCATTCATGCACACCTAGGTTGACTCCGTGTC
TTTGCTATTAAGAATATAACTGTGCTTAAGAGTAAGTGCTGTGTGTGGCTCATGCCTGTAATCCCAGCACTTTGGGAGGCCAAGGTGGGATGGT
CACTTGAGCCCAGGAGTTTAAGACCAAACTGGGCAACATAGTAAGACCCCATTTCTACAGAAAATAAAAAAAGGCCAAGCATGGTGGTGCATGC
CTGTAGTGCCGGCTACTCAGGAGGCTGTAGTGGGAGGATCATTTGAGTCACATTTATACCACTGCACTCTAACCTGGGCAACAAAGCGAGACCC
TGTCTCAAAACAAAACAGGCTGGGCGTGGTGGCACATGCCTGTAATCCTAGCACTTTGGGAGGCTGAGGTCGGTGGATCATTTGAGGTCAGGAG
TTCAAGACCAGCCTGGCCAACATGGTGAAACCCTGCCTCTACTAAAAATACAAAAATTAGCTGGGTGTGGTGGTGGGCGCCTGTAGTCCCAGCT
ACTTGGGAGGCTGAGGCAGAATTGCTTGAACCTGGGAGGTGGAGGTTGTAGTCAGCTGAGATTCTGCCACTGCAGTCCAGCCTGGGCAACAGAG
TGAGACTCTGTCTCAAAAAACAAACAAACACACAAACAATGCTGCAGTAAACATGTCAGTGTAAATGCCAGCTATCTTTCTGATAGATCGATTT
ATTTTCCTTTGQTTGTATACCCAGTAATGGGATTGCTGCCTCAAATGCTACTTTGATTTTTAGTTCTTCGAGAAATTTCCAAAACTGCTTTCCA
CAGGAGCTGAACTAATTTCCATTCTCATCAACACTGTTTAAGCATTCCCTTTTCTCCACAACCTTGCCAACATCTGTTCTTTTTTCACTTTTTT
TTTTTTTTTTTTTTTTTTTTTTTTTTTTTTTGAGACGGAGTCTCATTCTGTTGCCCAGGCTGGAGTGCAGTGGCGCGATCTTGCCTCACTGCAA
CTCCCGCCTCCCAGGTTCACGCCATTCTCCTGCCTCAGCCTCCCGAGTAGCTGGGACTACAGCCGCCCACCACCACCCCCCGCTAATTTTTTAA
AATATTTTTAGTCGACACGCGGTTTCACCGTGTTAGACAGGATGGTCTCGATCTCCTGACCTCATGATCTGCCTGCCTCGGCCTCCCAAAGTGC
TGGGATTAAAGGCATGAGTCACAGCCCCCGGCCTTCACTTTTTATTAATAGTCATTCTGACTCAGTTGTCAGATGGTATCTCATTGTGGTTTTG
ATTTGCATTTCTCTGATGATTAGTGATGTTCGGCATTTTTTCATGTTCGTTGGCTGCTTCTTCGTGTCCTTTGCCTACTTTTTTATGGGGCTAT
TTCCTTTTTTCTTGTTGATTTGTTTAAGTTCTTTTCTGQATATTAGTCCTTTGTCAGATGCATAATTTGCATATGTTTTCTCCCATTCTGTAGT
TTGTCCATCTGCTTCCTTGATAGCTTATTTTGCTGTGCTGAAGCTCTTTAATTAGCTCCCAACTGTCAGTTTTTCTTTTTGTTGCATTTGATTT
TGGGGACTTAATCATAAATTATTTGCCTAGGCAATGTCCAGAAGAGTATTTTCTAGCTTTTCTTCTAGGATTTATATAGTTTCAGGCCTTACAT
TTAAGCGTTTAATCCATCTTGAGTTTGTTTTTGTATATQGTGAGAGGTAGACGTCTGGTTTCCTATTTGGATGCCTTTTATTTCTTTCTCTTGC
CTCATTGCTCAGGCTAGGACTTGCCCCATACTGATTGCTGTGGCTCAGTACCATGCPGAATAGGAGTTGTTAAGAGTGGACACCCTGTTTTTTT
CCAGTTCTTAGGCGGAATGCTTCCAACTTTTGCCCGTTCAGTATGATATTGACGGCTGGGTGCAGTGGTTCACACCTGTAATCCCAAGACTTTG
GAAGGCCGAGGCAGGAGGATTGCATGAGCCTAGGAGTTCAAGACCAGCCTGGGCAGCATAGTGAAACCTTGTCTCTACTAAAAATAAAAAAAAT
TAGCCACACATAGTGGCATGCACCTGTAGTCCCAGCTTACTTGCGAGGCTGATGCAGGAGGATCACTTGAGCCTGGGTGATACAGGCTTCGGTG
AGTTATGGTTGCACCACTGCACTCCAGCCTGGGCAATGGAATAAGACCTTGTCTCAAAAAAAAAAAAAAALAAALAAAAAAAAAAGATATTGTC
ATAGGTGGCTCTTATTATTTTGAAGTATGTTCCTTTGATGCTTAGTTTATTGAGGGTTTTTTATAATGAAGGGATGTTGGATTTTATTGAATGC
TTTATGCTGATTGATTCTGAACATTGATAAAGAGGTATATATATATATCTTTAAATGATTTGTTCAGACTTTAATGGAAAGTACTAGTAAGAAT
AGCATGTAGAATTTCCAAATCACCCCATGTGCAGTGGGGAAATAGAGGAACAAGGAAAACTTCATCAATTGAGCAAAAATTGTTTTAAAAAATA
AGAAAACACGGGCCGGGCACGGTGGCTCACGCCTGTAATCCCAGCACTTTGGGAGGCCGAGGCACGTGCATCACGAGGTCAGQAATCGAGACCA
TCCTGGCTAACATGGTGAAACCCCATCTCTACTAAAAATACAAAAAATTAGCTGGGCATGGTGGCGGGCGCCTGTAGTCCCAGCTACTTGGGAG
GCAGGAGAATGCCGTGAACCCGGGAGGTGGAGGTTGTAGTGAGCCGAGATCACACCACTGCACTCCAGCCTGGGCAACAGAGCGAGACTCCATC
TCAAAAAAAAAAAGAAAACACTGTGTAGAAAACCAAAGACAAGAATAAAACCACTCATATCAGTGATTACAAAAAATATGAATGGGATAAAGTC
ACCTATTAAAGGCAAAAGGTCTCATAAAGTGAGAACAACAACAGTATCTAGCTATGTGTTCTTTATAAATGAAACACTAAAATCAAAAAGGTAC
CAAAAATTGAAAATAGAGGAAAGATTACGTCTGCCCATATTGAGATCTGATGGAGCTGTGAGACTTTACATTTTTCTTTTTCATATGCTTTTGA
AGTATTTATGAATGTCCTGGGATATAAGTCTAGGAATAGGAGCAATTCTTGAAACCATTTTATTATATGTTTTCACAAAATAATTGTAATATGG
TACTTGGTCTAAAGTGGCCAGTGTTTATTCTTTCAAATATTTTCGATTTATATAATTTCTTTCTTCCAAAAAGTTATCTTCAAGTCTTCAGGTA
GAATAAAATCATAGTAGAAAATAAGTCTTTCTGGCCAGCCATGGTGGCTCACGCTTGTAATCCCAACACTTCCGGAAGCCAAGGTGGGAGCGTT
GCTTGAGCCCAGGAGTACAAGACCAGCCTGGCAACATGGTGAGACCCCATTCTTAACAACAACAACAACAAAATAGCCAGGCGTGGTGGTGTGT
GCCTGTACTCCCAGCTACTAGGGAGGCTGAGGAAGGAGACTACCTTGAGTCCAGGAGTGTGAGGCTGCAGTGAGCCATGATCACACCACTGCAC
TCCAGGCTGGGCAACAAAGTCAGACCCCCAACTCAAAACAAAAACAATAAAGAGGCCGGGCACGGTGGCTCACACCTGTAATCCCAGCACTCTG
GGAGGCCAAGGCTCGAGGATCACTTGAGGTCGGGAGTTCAAGACCAGCCTGGCCAACATGGCGAAACCCCATCTCTACTAAAAACACAAAAATT
AGCTCGGCATGGTGGTGCGGGCCTGTACTCCCAGCTACTTGGGAGTCTGAGGCACAACAACTCCTTGAACTCGGGAGGCAGATGGTTGCAGTAA
GTGAAGGTTGTAGTGACCCAAGATCACGCCACTACACTCCAGCCTCGGCAACAGAACGAGACTCTGTCTCAAAAAAACAAAGAAAACAGTCTTT
TCTTCCTGCTGCTGCTGTTGTTGTTGTTACAAATATTATTAAATTTCCAGCCAATTGGCCATTTATTTTAGAATAGTATTAAAAAACAAACTTT
CCATATCAATATAAAATTTGTATAGCTTCCTAGAATTTATTAAAATAGGAATACTAATAGATTCAAAAAGGAAAATTTGTATAAATTAGTGAAA
ATTACCACCCTGTCCTTAATGCCCTATTTTAAGATTAGTCATTAAATAAATATTTACTTGCTGCCCCTGAACTCTGTGCTGGGTACTGTGAGGC
ACAAAAGAAGTACAAGATACTGCCTTTGCCTACTAACAATTTTATTCATACTTTGACAGCTTTCTGTGTTCAACCAGTAAATGTTTTCTTACAC
AGAGCTCTAAAACATGTTTAATCTGGTATAATTTACCACTAGGCACAGCAAAATAATTATAGCTAACATTCCTPTACTTTGTCTCTGTATATCC
ACACTTAACAACAGCATTTTCACATGATAGACACCCTGACATATGAATGAATGAATGAATGAATATCAGGTGCTAGATTAAAAGCTCTAATTCA
GTATCTAATTTAATCCTCTCAACAATTATCATTTAGCTAGTGTATTATCCACATTCTATCAGTGAAGAAACTGAGCTTCAGATCACTTTAATTA
CCTTGCCCAAAGTCATAATTGAAGTGGCAGAATGGGACTGGAATCCAGGTCTAACTCAAGTGCTATAGTGGTGTGAAGGGAAGACTGGTAAGGG
TTGGTGTTCATGGAGAATTTGTTGCTGCCTGTATACTTCTTGTTTAGATTTCCTGTTTTGAAAACATTCAAACTTCATCAACCAAAACAAATCA
TTTGCATAAATGTTATTCAGTTCCAGGAGCCAGCTTCCACGCCTGTTAGCATCTGTCCGTCAGTTCTGTAAGCAATCTTACAGTGGTGTGTGTT
CATCCTAAGTAGTACAAAACAGTGCAGAGGAGGAAGGCGGACAGCTTAGGGGAAATCTGACCACCATGCGTGTTGGAAGTATTTCTCTTGTTCT
CCTGCTTTTAGAAACCCATCTCTGCTTGCTTTTTCAGGGGGTTATTAAGATCCTACTCTCTTGGATACTGTGCTCTATTAGGCAGGAGACACAG
GCAGAAAGAAGCTCACTTGGCATGAATTAGGCAACACAGAAAACTCTTCCTCAGTGGCAGTAACCTGTCACAACCTGGTTTTCTCTGGCCACAT
AACCAGTCTTCTCCAGTAGTAATGCCATTACAACATTCCCATTCAGATCCTCATGACCCAGTAATTGTTTTGAGAATGAGTGCAAAGCTATCAG
ACACCAACTACTATAATTAGAAAAGGGAAAAATAGACACTTACCAGAGCTAATGTCTTTAGGCTGGAGCATTTACAGCTTTTTGTTTTTTGGTT
TTTAAGTTATGGAGCCTCTTTCTCAGTTTTGCTTCCTAAAATACAAATTGCATGGTTTAGGGGTTCTTGCATTTCATTTTTGTTACTGCTGTGC
ACTCTCTTTCCTTTTTTCCCCCCTATTTTAATCGGAGTACAAATTGCTGTCAGCACATCGAGTTCATGTGCCAGCCACATTCAGAACAGGGTGT
TCTGTGCTCTTCAAAACAAAATTGCTCTAGGGCAGTAAGAGAACAATAAGTCAGAGCTCCAGTTTAAGTGATGTTTTGCAGCTCGCTTGATGTG
GTTACACTCCGATTGCGCAGCTAAGCTTGGCTAATTTGGTTTGGTGACTTTTTGTGGGGATTTCGAGCACCTCCCTCCCCCTTTAAGTTACATA
GTCTTTAAGTCAACAAAAAAATGTTGTCTCTTTTTAACTTGTTTATTTCTTTCTCTACCAAAAAAGCCCTAAGTAGAAGCATCCTGTATGTACT
TTTATTCTCTTTGGGCATGTCATAGATGATTTACTTCCCGTAAATCATGCTGGTTGGGATTAGCAACAGACCTCCTGAGCCCTACAGGCTGCAC
TCCCAGTGAGTCACGATGGTTATTATTAAATATTTATTAAGTTAGTATTTGCACTAAAAGATGCCAGGTCAAAAAGCAACCACAGGGCTTCCAC
AGCCAGAGCTGCCATGGTTTTTGTTCTGTTTTGTTTTGGTAGGAATTTTTAAGATAACATTAAAAAGAATAAGTCGAGCTATAGGAGCTAGGCT
CATCTGCATCTGTTACCGCAATCAGGGCCCCTACTGTGGCAATCAGCAGTCTTTCCGTGGCTCCAAGTGGTGTGTGAGTTCTTGTCAGCAGTCT
CGTAGAACTTTTCACATCAGTGTTCCAAGTTTAGAAACCAGTTAACCCAGCAATGCCCTTTGCTACTTGTAGATGTAGAGAATCAATGGGCTGT
ATTCACCATGAGGCTGTGATACCTCCTTTTACTCTGACATTCCGAATGTTGTCAGTATTACAAGGCGATGGTTTTGGTTTTTTATATAAACTGG
GGTGTATATGTCTGGATGTATATCCGTTTAAGATTTTATAGTACTGCAGGGTCCAATTGTGAAGGGTTAGATTTACCAGGAAAAAAAAGATTAG
TCCACATGTAAAGTAGAAAATATTTATAAGAGCTATCACAAATGTTTACTGTCCTATTACAGCATCAGACACTAGTTCTGTCATTTATTGTCTC
TCTGAGAAGACTAGCTACAATGAATGTTTTGCTTTCTTAATTATGCCTTTGTTACAGTTTGACTTTTTTTTGGCATGGTTACTTCATTTATCTT
TCCCAACATGTTTATTAGTAGCCAATCTTTTTCCCTCACTAATGATGGGTTATCACATCTAAAGAGAACTTCTGAAATTACTCATTTAATTTTA
ATATCAAGTACATACATCAAGCTGAAAAACAGCCTAGGAAACTAACGCACAGGCAGGAATCCTTGCAGAGTCTCTGGATTTCCTGTTTTGAAAA
CATTCAAACTTGATCAACCAAAACCCACAACAAATGTACTGATAATTTCCTCCCCCACTAAATCTACTCTCCTTAAGGGAGGAAGATGATACTT
CCAACAATAAACTTCAGACAAAAAAAATCTGTTAACTAAGTTGTGAGCTACTACGACAATAAATCTCTATACTTGAGACATTCCACAATACATG
AGAAATTTACTTTGCTCAGCAAATCTTTTTTTTTTTTTATGAAAAGTGTTATTAGTCTAAATTTCTTGGGCAGAGTTTTAAATATTTAGAAATG
CAAACTTTTTAGAACTCTTAACCTCCTTAGAAATATCAATTTTTACTCCAGTAAAAATATCTTTTTTTTTTTGAGACGGTCTCACTCTGTCACC
CAGGCTAGATTACAGTGCCGCAATAACAACTCACTGCAGCCTGGAACTCCTAGGTTCAAGCAATCCTCCCACCTCAGCCTCTCGAGTAGCTGGG
ACTTATAGGTGTGCGTCACCACCCTCAGCGAATTCAGTAAAAATATCTTAAGCCTCAGACTCCCTGTAGAACCTCTCTAGTATGCATGTACAGT
CATATGCTGCATAACATTTTGTTTAACAGCAGATTGCACATAGGACAGTGGTCCCGTGAGATTATAATTCCATATCTTTACCTTCTCAATGTTT
AGATACACAAGTACTTACCATTGTGTTACAGTTCCCTACAATATTCAGTACAATAACATGCTATACAGATTTATAGCCTAGGAGCAATATGCTA
TACCACTTAGCCGAGGTTATAGTAGGCTGTTCCATCTAGATTTGTGTAAGAACATTCTAAGATGTTCCCACAGTGATGAGAATACCTGATCACA
CATCTCTCAGAATGTATCCCCATCCTTAAGTGAAGCATGACTGTATTTGAAAAAGTTTAAATTTGAATTTATTTTTTAAATGTTTTCCAATTCA
GAATGAACTTTCAATTTGCTACAAGTTAATTTTATCTAGAGCATTCTAAAAGTAAGTCTAACAGAAGGAATTATCAGCATTTATGAGTGTGGGA
CATAAACTAAAATGCCATATGCGAGCTCTACCAAGCCACTCCAACTCTAGCCCTTGTTTGGAGGAGCTGACTCAAGAGTTAGGCCCTTTCTAGT
TTGGCCCAAGTTTCTTAGACCCCATTCTQTGTCTGCAAGCGGAACACATCAAGATGGTATTAATACTGGTGAGTTAAAAAAGAGTAGTGAGTAC
TCACTTCGGCAGCACATATACWAAAATTGGAACAATACAGAGAAGATTTAGCAAATCGAAAAAAGAGAAGAAAAGGGTAGAACCCTTTAGAGCA
TAAATGTCCACGGCACATTTCGGCCCCTAGTGTCTACACCATTTCTGTTCATTCTTCAGTTTTTCTCATTTCTCAGGCCTGGGCTAGGGGTAAT
TCAATTAAATTATTAACTGCAGGAATTAGATTTGAAGCTGGTTTCTGGACTATACTGATATTGAGAGTGATATTACCAGGTCATAGAATCATAG
AATTTTAGAGCTTCATTGAGATCATGTAGTTCAAAAGTAGAATAAGTTAGATTTGAATTTTTATTTCGAAGGTAATAGTTGTTTTATTGGGCAT
GGAATGAGTATAACAGAACGAGTGATTTTTAGTCTCTCTACAAGTTGTTGATCTAAAGTAGATCAGCCAGTGTTAGAAAAGAAGCATTTTAGGA
AATCTCTAGATTTGAATATAGGCTATTATGTTACAGAAGTAGGAATAAAGGATTTTCTTTCTTTTTTATTTTPATTTATTTATTTTTTTATTTA
TTTTGAGATGGAGTCTCCCTCTGTTGCCCAGGCTGGAGGCTGGAGTGCAGTAGCGCAATCTCACTTCACTGCAACCTCCGCCTCCCACGCTCAA
GTGATTCTTCTGCCTCAGCCCAAGTACCTGGGATTACAGGCACGTGCCACCACACCTGGCTATTTTTTGTATTTTTAGTAAAGATGGGGTTTTG
CCATGTTGGCCAGGCTAGTCTCAAACTCCTCACCTCAGGTGATCCACCCGCCTCGGCCTCCCAAAGTGTGGGATTACAGGCGTGAGCACCGCGC
CCACTCACGATTTTCTTATGAAATCCAAATTATCATATTGCTTGGTGACTGTTATGCTGTCATACCAGCCATCCATTTTTCTTATTACATCAAA
AACTTAAAATAGAATACCCTTAATATTAAAAGTAGTTGTAAAGTTTCAGAGAATTGTTTTTCCTAGAAATAAAGAGTCTAGGCCAGACCTGGTG
GCGCATACCTGTAATCCCAGCGCTTTGGGAGGCTGAGGCGGGTGCATCACCTGAGGTCAGGAGTTCGAGACCAGCCTGGGCAACATCGTGAAAC
CCTGTCTCTACTAAAAATAACAAAATTAGCTGGGTGTGGTGGCACACACCTGTAATCCCAGCTACTTCGGAGGCTGAGACAGGAGAATTGTTTG
AACCCAGGAAGTGGAGGCTGCAGTGAGCAGATATCACGCCACTGCACTCCAGCCTGGGTGAGACAGAGCAAGACTGCGTCTCAAAAAAGTCTAA
CTCATCAAGAAAAAATAGATATCTGTATGCATTTTTTCTATTTTGTCTTAAAATATGAGAAAAAGCCTCTTATTTTAATATGAAATTACTCTGG
GAAAGAGCTTGGTAAATTGGGGTGGAACGGGGAATTAGQCCTCCCAAAATTGACCTCCTAATATTTCAGGGGAGGTTATTAAGTAATTTGATTT
GTGAAGTTATAGTAGACTATTGATTCTTTCACACTTTACATTTATTATTTCCAGTAAGGGATATAGCTAAGATAGTTATTGATGTTGTTGTCTT
TGGATTTACCATTTTTATTCAGATATATTTGTGAATGAGATTAGGACTCTTAAAGAAGAAGGAGCATCTGTATTCATTTAATCTTTGTATATCA
TTGGAGAAATAGCCATTTAGTTGGCTTTTTCGCCCAAGCATCTGAAGCCAAGATGCAGTTACAGATGTTTATATATTTGATATTAAATTTGTGG
CATTTGATAACTTTATTAAATAATTCAGCATCTCTTGACTTTGTATTATAGGTGAGGAAACTTCTGTCACATAGTAGGCTTAGAATAAACCTGT
GTTTCTGGTTTGTTACCTACTAAACTGTCTTATTGATACAAATATCAGTAATTACAAGTTTAATTTACAAATATGGTTGCTCCTAAACTTTCAG
ACTGGGTAGACTAAGCTTCACCAAGACACACTGTGTGTGTCTCTTGATGAAATGAGAAAGGGGAAATGGTATTTGCTACATTCTCGACCACTCA
TTTATCACTTCACTGAATTAGCCAGTCCTAAGAAGCAGCTCTGTTGACTCTCCTGTCTCCTTGCCCTCAGTCCACCTGGAAGCCCGCTGTGGTG
GAAGGATGTTTGACTGCAGAGATCTGGCATTCACTGTGGGCGAAGGAGAAGACCACGACATTCCAATTGGAATTGACAAAGCTCTGGAGAAAAT
CCACCGGGAAGAACAATGTATTTTATATCTTGGACCAAGGTAAGCAGCATACCAGTTTGGAACACAGTGAAGTCACCTAAAGGACAGCCATATG
CTTGCTTTTTCATTCAGTGTTTTGGCATTTTCACTCAAGTTAATCCAACAACGCAAACAATTAAATCAGGCTGCAGTTAGTCATCACGTAAATT
AATCCGCCTTACAATATCATAQAGGTTTGTGAGCATGTTTACTTGATTGATATATGTTCAGAACAGGTCTTACAQCCACTTTTTQCTTTTCTGA
TGATTTGTTTTGCCTGCAGTATTGCCAAGACAGGCAGCTATGGACCCCTCTCATATGTGGTTCTCTTACCCCCTCTTCCCTTTTCTCTAAGTTA
GGCAGGAGGCAGGAAAACATGGGGGTTGGGAGTCAAAGAAGCATTTGGATGATTTACTACCACTTGAACTTGTGCCACTTGTTCTTATACAGAG
TCCCTGAAAAAAAAAGCACATTCTTCCTGGATCTAAAGCCAACACAGATGACTCATTTTTTGTCTAAGTTTTTCCCATCATAAGCATATCCATT
AGCACTTTCAAGCTTAGAACCATCAGACACTGACAGTCTCTCTTGTGAAAACATGTTTAGTAGTATTTTTACATCTAAAATTATACCATGAATC
CAACTCTTCTGTCTTGAGAAACAGACCCCTGAGAGAACCCGGGGTGTTTTGATTTTGTCTGCATGAGAGTGCATCTGCTTATCTAGTGTATCCT
CCCTCCCGCTCCATGGATTTCAGACAGTGGGATTCTGAGTTACTGAGAATCTTGATGACCTGCACTTGCCAGTAQGTGATGTTAAGCCTTTTGT
GGTGTCATTTTATAGACTCTCTAGTAATCCAATGTCAGTTTACCTTTGCCCCAGCACTTGATCCATAATCATATATATATATATTTTGAGATGG
AGTCTTGCTCTGCCACCCAGGCTAGAGTGCAGTGGCATGATCCTGGCTCACTGCAACCTCTACCTCTGGGTTCAAGCAATTTTCCTGCCTCAGC
CTCCCGAGTAGCTGGCATTACAGGCACCCGCCACCATGCCTGTCTAATTTTTGTATTTCTACTAGAGACGAGGTTTCACCATTTTGGCCAGGCT
GGTCTCAAACGCCTGACTTCAAGTGATCCTCCCGCCTCAGCCTCCCAAAGTGCTGGGATTACAGGTGTGAGCCACCACGCCCAQCCCATAATCA
TATTTTTGAGGTGATCTACCCATGCAACTACCACTTCAAAATCCTTCCAAAGTGCTCTACCTTGAACTCTTCATAGCCGTCACTCAGQCACCCT
TTGTATAATGCCTTCTTTTTCTCTACCTGGTTCCTCTTTCCACTAACTATAGCCCCCACTTCAATGTGTCTTATTTATATTAATATTTACTCTA
TATTTGTAGTTTATTCTTAGGTGAAACTAATTCTGTTTCTAGGAGATACTCCTTATAAATTCAGATAGAGCATAGAAAAGCTATCTCTTCCATA
TCCAAATAATCTCTAATTACTTTTTTTTTTTTGGTAACAGGATCTCACTCTGTCCCCCAGGCTAGAGTCCACTGGCACAGTCACGCTCACTCTA
GCCTCAACCTCCAGGGCTCAGGTGATCCTCCCACTTCAGTCTCTAGAGTAGCTGGGACTACAGGTGTCTGCCACCACACCCGCCTACTTTTTGT
ATTTTTTGTGCAGACAGGGTTTTGCCATGTTGCCCAGGCTGGTCTCCAACTCCTGGACTCAAGTGATCCGCCTGTCTCACCCTCCCAAAGTGCT
GGGATTGATTACAGGCATGAGCCACTGCGCCAGGCCATCTCTGATTACTTTTAATCAGCATTTCAAAATTACAGTAGTTCTAAACTCAGAGAGG
TGGATTTATGTGTAATGTATATTTTCAAAATTTAAGCTCCCTTAGGATAAATTCTGTATGTATTATGACAACATAATGTTATAGGAACCAGTTT
ATATACATTACATTTTAGCCTGGCTGACAAGGCAATTGTGGGCACAGATGTCTCAAGTTTAGTGACAAAGAAAAATAGAAATTGTCTTTAAATT
TTTTAACGCAAACAGTTCTTTTCAGATTTAAACTGTTTGTTGTATCATTAGTGGTTACACTTTGCTCTGTGCGCTTGGCCAAGGCACTTAACCC
TCCTAAGCTTCAGTTTCCCCTCTCTAAATGGGGATGACAGTATCCATCTCATAAAGTTATGGAGAGCTAAATAAAATCCTGCACATATAGGGCA
CACATCACCCCCCAGTGTGTGAGCTTTTGTTGCTATTGTGACGGCTCACCTTCTCTTCCATTTTACCCTCGGACCACAAGAAAAAGGAAATCCT
GTTTAACATGAGATCAATCTTTCAGATCTTTTCAGGCATAAAAGTTGTCAATAGGTTTTCATAAATTTCTAGGTATAAAAGTTTATGAGTTTTA
TGGAAATAATTTAGCATATTTAGAAAGGAACATTATTTTTGTTCAAGGTGTTGGTATGTTGTAGAATTATGTGAGTGCATTCTCTGTTAATATG
AGACTGCTGCATATGCATTGCGTAGTACTACATATATATGTACAAAACCCCTACGTGCPATATATGTGTGTGCGTATGCATACTGTGTGTATAG
AAAACCCCCTACATGCTATATATGTGTGTGTGTATGCATGCTGTGTGTGTGTATAGAAAACCCCTACATGCTAAGCATGTTTTCAGTGCAGGCA
GTGAATTTACTTGTTTTATTCAGCTTCCCACTTTCATAAAACATTGCATCTAAATGTTGCTATAAGTATGGCTGTTGCAGAAAATATCTGCAAA
TAGCTCTTAGTGTCATCTGTATTTCATTCTCCAGAAAATTTCCAAGGCAGACCTTCTCTGTGACTCTGATACTCTGATAGACTCTCGGGAAGAA
TGTTTTCTTTCTGTCCCTCCCCCACCCCCCGAGACAGGGTCTCACTTTCACCTAGGCTGGAGTAGGGTGGCATAATCTGTAGCCTCGAACTCCT
GGGCTCAAGGGATCCTCCCACCTCAAGCTCCTGGGTAGCTGCCACTACAGGTGTGCTCCATCCTGCCTGGCTGATTTTTTTCATTTTGTAGAAA
CAGGGTCTTACTATGTTGTCCAGGCTGGTCTCAAACTCTTGGGCTCAAGCAGTCCTCCAGCCTCAGCCTCCCAAAGTCCTTCGCATTACAGGTG
TGAGCCACCACACCTGGCCTAGAATGTCTTCATAATAAGAAAGTCATAAAACATTTCCAGACAATCCATTTTTCTTTCTCCTCATTGGGACAAT
ATCTTCCCTTTGTATTGTTTTAAATACTGACACTAATAAATCTTTTTTGTTTTAGGTCAAGTGTATATAGTGTTAACTTTTTTTTCCGTCCTTT
CCTTATGTTTCTGTCACGCATGAACCCCAAACAAAAACACACGCTATTGGTCAGGCAAGGTGGCTCACACCTGTAATCCCAGCACTTTGGGAGG
CCGAGGCGGGAGGATCACTTGAGGTCAGGAGTTCGCGACCAGCCTGGCCAACATGGTGAAACCCCGTCTCTACTAAAAATATAAAAATTAGCTG
GGCGTGGTGGTGCATGCCTCTAATTCCGGCTACTCGGGAGGCTGAGGCAGGAGAATCGCTTGAACCCAGGAGGCGGAGGTTGCAGTGAGCTGAC
ATCATGCCACTGCACGATGCCTGGGGTGACAGAGTGAAACTCTTGTCTCAAAAATAAAAATAAAAAAACAGACTACCAAGAGCCATAATGTCTA
GTTATGAGGCAGTATGAGTCCAAAAAACCTGATTTTCTGGGGACACACAGGGTCTCAATTTAAACCTGGCTCTACCACTGCCTGTGTGTGACTT
CTCAGGAAGCACTCTGTGTCTTTATCTGCAAAAACCGACCAATTGGTAAGAGCTTGTAGTCCAAGTGCTTCTTATATCCACATTAAGGCTTAAT
GGTCATTCATAGTCATTATTTGTATCTCTATTCTCTGGAGGAAACTCTTAATTCAACGGCATTAACGCTGAATTCCTTCTGTTACCATACTCTA
GATTATCTTTTGCTTAGGTCTTCCGATTCCTCTTGTGQTGTTTTGTTTTTCAGTCACATCAGTTCCGTGCCCTGGTTCCTTCTTCACCCAGGCT
GACAACTTTCTGTGTAATATGGAAGTCAGGGTAGGTGCTGCCAGAATATCTTGCCGTGGGCTTCCACCTCTGCAGGATCATGTCCGCCACACGT
ATCTTGACTGAATCGCTTCATTTTAACCTGGGAGAGATTGTAGAAATAATATTTTTCTTTAGTCTTCTTTAATCCCATCAATAATAGGACACTT
GGTGCCTCCCTTGATACAGCCCCACATTCCCCACAGCCAQATTTTCTCATCCTCAGCACTGTTGACACTTTGGGCTTGCTAATCCTTGGTCATA
GGGGGCTGTCACGTGCATTGTAGAATATCCAGCAGTATTCCTGGCCTCTTCCCACCAGATACCAGTAGCACCCCCATAGTTGTGACAACCCATA
ATATTTCTAGACATTGCCAAAGGTTTCCTGGAGGGAAGAGGAGTTAAAATCAACCTCGTTGAGATCTGCTGGTCTAAGGCAAATGCCAGATCAT
TTGTTGCATTTGCAACTGCTTAGCACAGGACTCCAAATATAACATGCTCTCAGAAAATATGTGCTTAGCAAGGACTCACCTACCTACCTGGGTA
GTTAAGCAATTTTTTTAAAATTTATTAGATGGCAGAAACAAGGTAGTAGGTATATACATCTAAGACCTCAAAGTAGAAAATTAGGCATCACTTC
TTAAAATTGGTCACTGGTAATTATGTCTTTGTGGAAGATACCCTTTGCCCCGCTCCTCATTCCTACCAGCTCTCGTATCTCTATTCCTCTTTTT
GGCTCCAGCTTCCATTTTCTGTCTAACAGTGGAGGTGAGTGAGTAAGCATTTTTATTCCTGGAAAATCCAGGCAAAGGGTAAAGAAAGTGGTGC
AAGCAACAAAATTTGAGTGTTCCCACTCTAGTCCCTTTCCCCCACAGAAAACTCGACGTATTAGATTAAGTCGGTTTTGACATCTTTCAACCTG
TACCTTGAAAGCTTGAGTTTACTAAGTTTTTTGTAATACCATTTCATATATTAAAGAACAGCTTTGCTTGCAACAAATGGGAACATAATATATT
TCAGTGAACACTTGACTAATATGCTGGGCCTTGTTCTAAACAAACACTGGGAAACAGGTGAACCAGACAGAGCTCTGCTACAGCTCCCATTCTA
GTAAATATCTCCTTCCCCAATACATTTAATTTGCTGGAGATCTTACATGATCCCAAGAAAACAAATGTATATGTCCTCAGTTATTTTTTTCAGT
AAGTTTTCGCTAAAAGCTATAGAATCCTACACTCTGATAGCTATAAGCCATAAGAATATTTCTCTAGAGGAGGTCTCAGAACTTGCTAGGGCAG
CTATTTTTGAGTATATCATTATGTTATTTGTGTTTCTTCATGTGCTTTTAAAAAAACTTAAATAGTGTGAAAACTTTTCTCTCCTGATGTGTGA
GCCTTTCCCCTTCACTTATGTGACTGCAAGTGGTACTACTAGAAGTGACAGCACTTTAAAGTTAAAACCTTATTAAAACATACCTCCCGTGAGT
ACCACTCTAACAATTACAGTGGCTGTGTCTTCTCTGTCCTTTTTGCTGATAAAACTTAGGGGAAAATTCCTGGCAGCTAAATCATGGCCATTTT
ATTAAAAAAAAAAACTAGATATGAAATCAAGTAGAGAAAAAAGACAGTGACATTTAAAATCCCATGAATCAAGCTGCCCCAAGGAGTACAGGTG
TTGCATTACATGTGAATGTGAAGGAGAAAAGAAAACTAAACCTAGCCTCGTTTCTAAGAAAACTGTAGCAGTGAAGATACTTCACACTGTATCT
TCTGGTCAACATCTGGTTTGTCTTCAGGGTCTCTGACACATGTAGCTTGCAAATGATAGCTCTGTTGATGCCTCAAAATACTGAAGGAAGTGCC
TGGAAATACACCAATCCTTTTAAAGTTTTATTTTTTACTCTCCATTTCAGATATGGTTTTGGAGAGGCAGGGAAGCCTAAATTTGGCATTGAAC
CTAATGCTGAGCTTATATATGAAGTTACACTTAAGAGCTTCGAAAAGGTGAGAAAATACCAACTTCCTTAAAATTATGGTAGGTTTCTTCTCCA
TCCACTTCCTATAAATCAGTATCTTATCTTAGTAGGTGGTATAGATACATCGTCTTTATTCATTATCAAGTTTGTCTCTTGCCTCCAACACTGC
TGCTGAACAATAGAACAACTGACATACCACAGCTGATATACAACCAACGACCTCCGTGTTCTTATTTTGTTCCTATGTGTCAGCTTTTTTTTTT
TTTTTTAACTTTTTTGGGTAGAATCCAAGACTTGTTCCCTTATTTGTTCCTAGACTTGTTCTTTTTGTACTTTTTTTTTCCTGGGTTGAGGACA
GAAAGGTTCACGTTCTGCCAAGTGGCTCCTTGGGGGAGTGGGGTGCAGCTTTTCAGAGCTGAACGGATTGTACCCTTCACAGGAGTCACGAAGA
GGCCTGCACTGTGGCTCCAAGTTGTTATCCCAACTGCAAGTCCTCGAGGGCCACTAGGCTCTGTGACCAGAGATACAAGAAAGAAAGCAGCTAA
CCCGAAGCCATTTTTTTTTTATACTTCTCCAAGTAGAGAAGAGAGAGAATTTGTCAGTTGCCTTGGATTCCAGAAACAGCATGCCTCCTTGAAC
ATATTTCCATCTCAGTTTCACTCTGGAGGCTCCCAGCAGCAGCCGACCTGGACACACACAGCATATACCTCAGCTACGTATGCTGTTGTCTTCG
GCCACGTGCCCCTGGCATTTCCAAAGCCCCACCCTTTGTGGAGCTCAAGGACTGCAGTTGTGTTCCTTTTTTATTCCTGCACAGTGCTACCTCC
ACGTTCTGTACATTCAACTTTTTTTTTTTTGAGATAGGGTCTCACTCTTGTCACCCAGGCTGGAATACAGTGAGCGCGATCATAGCTCACTCCA
GCCTCAACTTCCTGGCCTCAAGCAGTCCTCTCACCTCAGCCTTCCAACPAGCTGGGACTACAGGCACACGCTACCACACCCAGCTAATTTTGTT
TATTTTTTGTAGACATGGGGTCTCACTGTGTTGCCCCAGCTGGTCTCAAACTCCTGGGCTTGAGCCATCCTCCTGCCTTGGCCTCCCAAAGTGC
TGGGATTGTAGGGGTGAGCCACTGTGCCTGACCACATTAAGCACATTAATATAAATGGAAACATGACCATTTATATTTTGTTAGATTCAGAAAA
GAAATCATTGTCCTGTGGGCCTGTACCTTTGGGCTAAGAAAGWGAGGGGAGGCCTGCTTTGGGATTCACCCCCAGTAGTGACATTCCTAATCAT
ACCTCTTCTACTTCATGTTCCCCCAAACAAACATATGTCTGGTTATGACCACTGATTGTTGTCATGTAACCTCTTCTGAAGGCCTGCTAGCTCA
GCACTGATCCCCCAATGCTGTTGTCATAGCATATGGTGTTTTTTTATTGTTAAAGAGAGAGAAGATAATCTTTAGCATGTGGTTATTTAGACCT
ATGGCTTTACTATTTCCATTTTTGTAAAGCAGCCTCTTGCCCCTTCCTTCTCGTGGCCCAGTGGACATTGCTTATTTAGGAGCACTCTCCCAGT
GGTGTGCCACAGCAAGCCTGTGCTCTGTCATCACTCCAAGAGTATATTCATGCACTGCTGCTTGGTATATTTGAAATCACATGAAGGTATATCA
TGTTGTTTGCGGCAATGTGATTTAATCCTCTGCCTTGTTAAGAATAGACTAGGCCAGGCTGGGCGCTGTGGCTCACGCCTGTAATCCTAGCACT
TTGGGATGCCGAGGTAGGTGGATCACCTGAGGTCAGGAGTTTGAGACCAGCCTGGCCAACATGGTGAAAACCCATCTATGCTAAAAATAGAAAA
ATTACCTCGACGTGGTGGCAGGTCCCTGTAATCTCAGCTATTCGCGAAGCTGAGACAGGTGAATCGCTTGAACCCAGGAGGTGGAGGTTCCCGT
GAGCCGAGATCATGCCACTGCACTCCAGCCTGAGAGACAGAGCAAGACTCCGTCTCAGAGGAAAAAAAAAAAAATATATATATATATATATAAA
AATATATATATATATAAAAATATATATATAAATATATACACACACACACATACACACACATACTTATATTCCAGGCATGGTGGCTCACACCTAT
AAATCCCAGCACTTTGGGAGGCCAAGGCAGGATTACTTGAGCCGAGGAGTTCAAAACCAGCTTGGGCAACATAGTGAGACCCCCCTCTCTGCAA
AAAAATTTAAAATTTTCCTGCGTCTGGTAGCACATTCCTATATTTCCAGCTACTCAGCAGGCCAATATAGCAGGATGGCTTGAGCCCAAGAGTT
TGAGACCAGCCTGAGCAACATAGTGAGACCCCGTCTCTACCAAAAAAAAAAAAAAAAAATTAGCTGGACATGGTGGCACATGCCTGTGGTCCCA
ACTACTCGGGAGGCTGAGGCAGGAGGATTGCTTGAGCCTGGGAAATCGAGGCTACAGTGACCCGCGATCQTGCCACTGCACTCCAGCCTGCACG
ACAGAGCAAGATCCTGTCTCAAAAAAAGAAAAAATAAAAGAGTATCCTGTCTCAAAAAAAGAAAAGACAACAGAGCAAGATCCTGTCTTAGAAA
AAGAAAAAATAAAAAAAAATTCCATAAAGACTGAAGCCAACCTAAGAAATACAAAGAGACTTATTAACTAGGTGTTCTTTGTTTTTGTTTTTTG
GGTGCTAAAACCTGGAATTGAACCATAACTAGGTTTTTGAGTAATTTGAGCTACATTATTAGGAAGCAGAATTACATGAGTTGCCAGAAACATG
AGTAACCCTTTTATTATTCACTTTTAAAAAGTACAGGATAGTCACTGGGCGCGGTAGCTCACGCCTGTAATCCCACCTGAGGTGGGTCGATCAC
CTGAGGTTGGGAGTTTGAGACCAGCCTGACCAATATCGAGAAACCCCGTCTCTACTAAAAATACAAAAAATTAGCCGGGCATGGTGGTGCATGC
CTGTAATCCCACCTACTCCGGAGGCTGAGGCAGGAGAATCACTTGAACCCGGGAGGCAGACGTTGTGGTGACCCAAGATCACACCATTGCACTC
CAGCCTGGACAACAAGACCGAAACTCCGTCTCAAAAAAAAAATAATAATAATAATAATAATAAAAGTAGAGGATAGCAGGGGCTGTGACACCAG
GAACACACACTCGTTATCTTTTGAATACAGAGAATTTTAGTTACCCATTTAAACTTCATAATTTATATCCACTAAATAGCACTGACCACACCCA
CTGTCACTTTTCCTAAACGCTCCCAGATTGTTCTCTTGACTCTGGTCCTTCTAAATCCAGCTTGTAAATGGCAACTGCAGTTATTTCTGGATTT
GTATCTGTAATGGTGGAAGTAATAATACACACAATACAAGCTAGCAGATGATAATGAAGTCATTTATTTATGGCTCTAAAGTGATATTTTTGCA
TGAQCTTTACTCCCAATTATGTTTTCCTTGAAGTCCCCCCCAGTATACATCTGCAAGTTGATACAATTTAGCAGTATTATTAGTATATCTCGTT
QTTTTAGAGTGGCAAGACAACCTGACCCAGAATTTTGAAAATTCTGTATQAATATACAAACTACAACTTGACTACAGTCATCTTCCCATTTTAG
AGTTGAAAAACAGGCTTGAGAGAAACACCCTAGTGGAGAGCTTTGCAGAGGGAGATAAGGCATGGCCTTAACTTTGTGTGGGTTCACCTTGTAT
TTCTATATATTCACTTTGTAGCAGTGCTTACCAGAGTACGTTCCTTACTGTGTTTTTGCGGCTCCTCTCTCTTGAGCAACATTGTTCAAGCTAC
AGCTTGGTCATGATGGAAAGTGAGTTCTTCAAACAGTATGAAGAAGCACTGGCCTAGCAGTAAATTGGCACTGACACACAATTTCAACAGCCAC
AGTGTTTTCAGTTTGCAGTATCTTCTAGAATTAGCTGCTCTTTGTTCCAATAGGCCTCAACTGATGCTTTTCTCTCTGTTCATCTAACCATTGT
CACAAATTTACACACAGGCCAAAGAATCCTGGGAGATGGATACCAAAGAAAAATTGGAGCACGCTGCCATTGTCAAAGAGAAGGCAACCGTATA
CTTCAAGGTGTGTGAGTCCCTATGCAATCCTTTCCATCTCAAAGAATTTATTCAAGTGTTGGTGAATTGTGCTGATATTCCCTACTCTGAATTT
TGTCAACACTGCTTGCTAAGAATAAGCTGCAATTTTTATAAATCTGTCATTTTCAATATGAAAAATGGTGAGAAAACAATCATGTGGAAAAAAA
AAAAAGTACAGATTCCTCTTTTACACATTCTGCTCTAGATTGTAACCTTCCATACAGGCAGACCAGGTCTGGCATCCCCACCCACCACAGTATC
ACTCACTCCTAAAGCATCTTGCTTGGTACATAGTTGGCACTCAACAAGTGTTTTTGGAATGAAAATAAGAATGAATAGAATAATTGTAGCAAAG
AATGGAATTCATAAGGGTAAGTTTCACATCATTTTTCTTTCTTCCTTTTTTTTTTTTTGTATGAGACACAGGGTCTCCCTTTCTTTCCCAGGCT
GGTCTCAAACTTCTAGCCTCAAGCAATCCTCCCACCTCAGCTTCCCAAACTGCTGAGATTACAGGCGTGAGCCACTGCACCGGGCCCATCTTCC
TTTAGACTGTCTTGATGAACTCACTAGAGCATATCATAAAAGGAGAGAGCACATGCACAAATTTCAAAACAAACTCACAGGAGAGTATTGATTG
CTTCTAATGGCCAAGATGATTCCTGCACACTTGCTTTTATGAGCAATCTCCGGATATTCCTGAATGATATTCATCATAAATTGCTTCTCAGAAA
CCCTTCTTGGAGCAACTAGGAGTGTGATATTTTCTCACTTAGATCTTAAACCTCTAGATGTAAGAAAAAAAGAAACCGCCCTTCATTCTGTTTT
AATCAGTQTTCTACCCATTGCTTCCCCACCCCAAATATATGCCTTCTCCATGTCAAGCAGAAAGGGCTTAGTCATCAGAATTAGACGGAGAATG
GGAGTTCTGTCAAGGTTTCTTTCGTTTTCTGCCACTGCAATACAGAAATTAGATGTTTTTCAGCAGTATAAAGCAGTGAGGCATAAGTGTACTT
TAACAAGTCTTTATGGGGCATCTGCTATAAGACATGCTATTATTAAGTTACAACCTTGTCAGGAGGGAGGAGTGGCAATGTTAATCCAAATGAA
ACAACTAGAGAATTATGTAAGAATATATAATCAACCCAAAATTAACACAGCCAGAGGGCACCATGTCTTTTCAGAATTTCAGCTGAGACAATAT
TGGGTCATGAAATCAGTTTTGTGGAAGTGTATTGCAATATGTPATAAGGATCAGTTTTGTTGCATAAAACTTTGCTTGTGTATGAGTAAGTACT
GGATTGCATATTTCTTACTCTGGGTGGTATTCAAACAAATTTTAAATCATGGGTTTAGATAACTATAAATATATAAATATGTTATTTATCATTA
TTTACTATGTATACTAAACTCTTATGGATCACCAGAAAGCATGGGGGTATGATAGAATAAGCTCAGAATTGGGAAAAATGGGCTTTATTGCTCT
TCAAATGATTTAGTCATTTAAGGTCCAAAGAGGTTATATTTTCAAGTGAAAAATAGGGGAGTTAGATGTTCCCCAAGTTTCTTTCCAGCCTGAA
TTTCTGTGAGTTTCTAAACTCTCAAGAACAAGTGTTATTTTTTTGGTTTTGTTTTGTTTTTAATTCTAATTGAGGTATATTGGATACTTCTAAA
CTATTCAAGTAAGATTCCAAGTGGGTTATTTGTATATCCAAATACATAATACACTAGAGGGAAAAATGTTTCTACAAAATTTCTTAAGATCGTG
AGTCCTTAACAGTATTTTCTATATTTCCGAAGTCCATAAAAATCCTTAACTATATTTTATATATTTCCTCAATACTTCCAAATAGAATATTAAC
AGTTTGGGAACATTTGACGCTTTTGGCCTTTGACTAGAATTATATCGATTCAGTGTACTAAAACAGCTCAGCTATTCAGAGGCTCTAATCGGTG
CTATGATACAGAATTTTGGAGAACATTATTGGTAATGAAATAATGTTATGAAGCCATCTGTCATATAGCTGTGTTCTGAATATATACAGAGAGA
TGGCCAGAATTAGTGATCAGTAGCAGAAAGCAATGAGTGTCTGTACAGTTACTTAACTTTGTTGCTTATCAGCATCACAACTGCAAGAGTGATA
CTATTATGAACCTACTTAAAATTACCAATCTTTGGATAAAATCTGGATCAGCAGTCACACACATGGAAAAAATTAPTTTTAACATGGAATTTCA
TAGGCAAAATTTTTCAAAACATTAGTCCATTGGGGAAAAATTAATGAAAGGTTCAAAACCACTGACCTGAGACTTTTCTCCCAGAATGTCTCTG
AACATTTTGTACCACTAAAAACTTCCCTGGAGGCACCTTTATAATTATAGGTAAAAGATCCCAGCAGTAGTCCTAGCGTTAGCTGTAAAACTTC
AAAATGGTTAGTAGATAGTTTCCTGTACATATTCCCTTCCCAACTCAGGGCAGGTTTCAATCTGCCATACTGCCCTCCCGCGCCTTGFCTTTCC
AGGGTGTCTGACTTGGCATGTTATGCATTTGCACAGAGTCCAGATGTGTAGCTGAGAGCAGAGATCGCCTGAACTAAAGATCTGTATTTCATGC
CATCTGCATATTGGAATTCTGTTTCCATTGATCTGTTGGTTTGGTTTATACCTAAATGGGTAAAGCCTCTCTCCTGACTAGGATTGAATTGTAC
TCAGCTTTTAAGTGGGAATGATTTTGCCATTGAAGATGTAAAGTCCTCATTTCAACCTCTGAATACTAAAGAGAAGAAAAATAGAGAACTCTAT
TTGGCTCTGAAAATAATTATTAAGTACAGGTAACAGCATAATATTAAGCCAGGCATTGTGCAAGCCAGTTAAACAGACACCATTTTTGCCCTCA
TAGCTGACAGTGTTCTGCAGGACAGGAGCATTAACTCACACCTGGTCAGTGTTTTTCTTTTTTCTGACTTGGCAGCAGAGTGGCAAGAAGGCTA
AGAACACTGAAGTGATGTGGGATTCTTGCGCCCCCTTGTGCTTCTCCTGGGAAGAAAGCATTTCTAAAACAAACTAGTTGAGGAGAATTGTTCA
ACCTCTAAACTCTTCAGATAAATCCGGCTTTGCTATATTTTCACCACCCTTGCTGTTCCAAGATACCACTAATGTGATTTAGAAACAAGCCGTT
TATGACTCTACCATTTGATGTTTTAAAAAAAATTCCTAAGAAGAGTCCACCCTGTCTTTCATCTCTTTCCTTTAGTGCAGAATTTTTCAACCTC
AGTACTATCAACATTTGGAGTGGGATAATTCTTGATTGTGTGGGGCTGTCTTGTGCATTGTAGAATATTTAGCAGCATTCCTGGCCTCTGCCCA
TTACATCCCAGTAQCATCCTACCCACTTATGACAACTAAAAATCCCTCCAGACCTGCCAGGTGTTCCCTGGGGGGCAAAATCACCCCCCAGTTG
AGAACTGCTTTAGTACCTCAGAAGCAGGCAGTACAAGACAGACTTTTCGCTAATTGGAAGTTCAAGTCTAAATAAAGTTACATGATATATATGC
AAGAGCTTTCAAAAAATTAAAATGCCCTATGTGAAGGAACTATTAAGAAGTCTAATACCTGTTTTTTGTGTTTTGTTTTGTTTTACCATGGCCT
TCTACCATTCTGATTATTCAGAACTTATTGAAATGTTAAGATCTAGGTTTTTGTCCTCCCTCGCTCTTCTTTTCTTTCCCTATACAATATATTA
GGAGATAATCAGAAAAGTTAAGTTAATCAATGCATTAGAGAGGGAGGAGGAGAGAGGATGAAGAAATGAGGCATGACAGGGGAAATGCCTGTCC
TTCCTATCTCATCTCAGTGAAACAACACTTTTCATTTCCACCCACACATTTTTTGCTCCCAATGATAGCAAAAGGTTTTGTTGTTGGTATTGTT
TTTAGAGAGAGAGGGTCTTACTCTGTCACCCAGGCTGGAGTGCAGTCGCACAAGCATAGCTAACTGCAGCTTCGAACTCCTGGACTTAAGAGAT
CCTCCTGCCTCAGCTTCCCAAACTGCTGGGACTATAGGCGTGACCCACCACACCACTTTAGAAAAAGTCTTGATTCCATATTTACATTCCTTTG
ATTTCAATATTTACTCAACAAACCTTTATTAAAGACCTCTTGGTGCTAAACCCTGAATATTCCAAAAGGGAGAGGACAATGCTGCCTGTCCTCA
AAGACCAGCAAGAGAAAAGGCAGTGACAGAAAGAAAATGCCGACCTGAAACTGGTGAGGACTTTATTCTTTTTTTTTTGTAGCATCCAAAAAAG
GAGTGGTAGGCCTAAGACTATGACAAAATTTAAGTATTGTCTTAGCTTGGGCTGCCACAGTAAAATACCATACACTGGGTGGCTTCAACAACAG
GAATTTACTTCTGGAGACTGTAAGTCCCAGATCAAGGTGTTAGCAGGATCAGTGTCTGGGGAGGGCCTCTCCTCTGGTTCCTTCTAGCTCCCTT
CTTGCTGTGTTCTCACATGGCCTTTCCACATGCATGTGAGTGGAGAGAGGGCAGAGGGCAGCAAGCACCCGAGTTTCTTCTTATAAAGGCTCTA
ATCCCCATCATGAGGCCCCACCCTCATGACCACTCTAACCTAATTCCCTCCCCAAATGCTCCATCTGCAAATATCACATTCGGGGTGAGGGCTT
CAACATAGGAATTTAGGATGGGGGACACAATTCAGTCCATAGCAAATCTAATTTCCTCAAGAATTTCAGCATTTAAAATAAAATGCCCTGGTTT
CGTGTTCTTATGAATGACCTGTATCACACACTTCTGAGGCAGAGAGGGTGACAGACTTTGATTTTAGTGCCAGTCTTCCCTCTCCTAAGTGAGT
GACTTCGGAAGAAGGATGTTCGAGGGATGTAGCCTGGCCCTCATTCACACCCAGCCCCTCCAGCTGGATCAGGTCTGCCCCATCTTCATCACCA
GAAAATCCACACCAGGGCTTCCTTTCTACAGGCAGAGCTCGTTTCTTACATCACCCACTTGAAGTCCTGTATTACAGGCATGTCCTCTTAGACC
TGCTCTATGGTTTTTCTCTTACTCAGACTCCTTGCGTGAGGGTTGGAGTATTTGTGGGGAGAGACAACTCGGCAGTGAGATGTACCCGCTGCTT
GGGCTGTGGTAAATCCTAGTAACATGGTCTTAACACTGTTGGTCAGGCATTGAATTTAATGTCCTAATAGGTCTGGCTGCTCCTCTGCCATCTG
GCAGGATGTATCATCTGTGGTTTTGAGCTTATCACCTCCTCAAAGGAGAATCCCCACTTGTACTCCTGTTTCTATTTGCGTATTTACCAGCCAT
TCCCCAGTGCTTTCATTTACCTTAAAATCACAGTGGACACATTGTCTCAGCCCTGGAGGGGTTTTCTTTTCTTTTGTTTTGTTTTGTTTTTGAG
CAGTCTCGCTCTGTCACCCAGGCTGGAGTGCAGWGGCATGATCTTGGCTCACTGCGTACCTCTGCCTCTTAGGTTCAATGCAGTTCTCCTGCCT
GCCTCCTAAGTAGCCGGGATTACAGGCATATGCCACCATGCCTGGCTCATTTTTGTATTTTTAGTAGAGACGGCAGGCTGGTCTCCTTACTCCT
CACCTCAGGTGATCCACCTGTCTCGGCCTCCAAAGTCCTGGCATTACAGGTATGAGCCACTGTGCTCAGCCCCCAGCGTTTCTCATATGAGCAT
GTATATATATATATATTATATATATATTATATATGTTTATATTTATATATATTATATATAACTGACAGACCTTTCTGATGTATATTCTTTTATA
GTTGTTATTATGTTGTATGCTACTATCCASACTATTCATATTCCCATTGCCTATAGATCAGGGGTCAGCAAACTACAGTTTGCTGGCCAAATCT
GGTCCCTGCCTGTTTTTGTAAGTACAGTTTAACTGAAACACAGCTGCACCCATTGATTTACATACTGTCTAGAGCAGCTTTTGCACTACTATTG
ATAACAATTTAGTAGTTGCAACACAGACTGCTTCAAATAGGATTGGCTGTGTTTGGAACTAGCACTTCCCAGATGTTGTATTTGCTGTGTAAGT
GCTGAAAAGAAAAGTAACTTCCTTTTCATTTTTTTAGTGGCATAGTCTAAAACTGCTCACAATCCTTTTGAAAGACTAAAGAATAGGCCAGGTG
TGGTGGCTCATGCCTGTAATCCCACCACTTTCGGAGGCCAAGGCAGGCGGATCACTCGTCAGGAGTTCTAGACCAGCCTGGCCGATCATAGTGA
AACGCTGTCTCTACTAAAAATACAAAAAAAAATAGCTGGGCTTGGTGGCGGCCACCGGTAGTCCCCAGCTACTCGGCAGGCTGAGGCAGGAGAT
TCGCTGGAACCCGGGAGGCGGAGGTTGCAGTGAGCTGAGATGGCGCCATTGCACTCCAGCCCTGGAGACAGTGCGAGACTGTGTCTCAAAAAAA
AAAAAAAAAAAAAAAAAAAAAAAGACTAAAGAATATTAGCACCAAACAACATAGCCCAGAAGCCTTGCTGTACTACACCTGCTGATGGGATGGA
TGGAAATGCTGCAGAGCAGCACTAGGCAGTGCTCAGGCACTGCCCCAAACCAGCCCACAGGCCATCGAGGCCTCCAAGGGAGTGGGAAAGGTAC
CTGCAGAGCCATCTCCAGAAGGAAAGAAACAAACCAGCTGTACCAGGCTTTGCTAAAGGGGTTGCTTCACTTTCAGGATTTGCTTTTCTTTTGT
ACCCCCTCAACAGCTGAGTACAAGTAATGACGCTTTGGTTATCCTAGGCAGGCAAATACATGCAGGCGGTGATTCAGTATGGGAGATAGTGTCC
TGGTTAGAGATGGAATATGCTTTATCAGAAAAGGAATCGAAAGCTTCTGAATCATTTCTCCTTGCTGCCTTTCTGATCCTGGCCATGTGCTACC
TGAAGCTTAGAGAATACACCAAAGCTCTTGAATGCTGTCACAAGCTAACATCGTGTCTCCTGTGAGATGGACTTTAGATACTCCAGATCTTTTC
TCCATCTCTGTCTTTTGCTTCCTCTCAGCCAAAGGTTGCAGGATTTTTCATTTTCCTTTTTTTTATTTATTTGAATGTTTGGGATTATTTGTTT
CTCAGTATGAATTGGGAAATTCAGCAAGATTTTTATTACAGTCAGACTTTTATGATACCTAGACTGTTTATCATAATGCATTCTGAAGAAAAGT
TTTGATTAAATTAAATATTTTTATCCACGACCTTCCTTTAATAACTGTAGTATTTATATCAGAGTAGACGTATAACTCCTCAGGTTCAGTTTAA
TAAGAGAAGATTTAACTTAAGCTTTTCTGGACTTTAACAGACTGCAACTGTTCATATAAAGCATATATATAGGTCAATTTTTATTATAATCATT
TATTCACTGATATGCATAAATAATATGAAAGGTCCACAGAGCATCAGCACCCTTTTTATTTTTACATTTGTCTTATTTATACTATCTTTGGTCT
TTTGAGTGCTTACACACAGTACTCTATAACAGTGGTTTATCATCTGTATCGTTATGTATCGTTCTGATATATTTATACACCAGCCGATACTATT
TTAGGTATCTGGTCTCAGAGTGAACAGTATATATTATACTGTGGGTAAAAGTAAAAACTAATCATTGGATGTATCTTAAAGGTAGACATAAAAG
GATAAGAAAAGCAAGGCAACTATTTTGTACATTCTTTGTGAGATTATTTTTGAATTTTGTGGATCAGTGTTTCCTATCGCATCCTCTCTCCCAA
GACCAGTTATTAATTTTATATGTATTTTTTCGTTTTTTTCATACAQAGCGGAGAGAGAGAGAGAGAGAGTGTGTGCGCGCGCGCGTGTGTGTGT
TTTGTTTGTTTTAGAGACAAGTTGTTGCTATTTTGCCCAGGCTGGGGTGCGGTGGCTATTAATACACATGAACATAATGTACTACAGCCTCAAA
CTCTGGGGCCCAAGCTATCCTCCCACCTCAGCCTCCTGAGTAGCTGGGACTACAAGTGCATGCCACCACACACCCTGCTAATTTTTAAATTTAC
TTTTGTAGAGATGGGGGTCTTCCAAAGAGCTGGGATTAAAGTTGTGAGGTACCACACCCAGCACAGCCTTGACTACATTATTTTTCTGTCTTCT
ATTGACACAAGCACTCATATAGCATCACTATTAACCCTATCTTTCGCAATAGGAAGCCTGTTCTCCTTATTTTCCTCTTTTTTTTTTTTTTTGG
AGATGGAGTCTTTCTCTGTTACCCAGTCTGCAGTGCAATGGCACAATCTTGGCTCACTCTACCTCCACCTCCCGAGTTCAGCCATTCTCCTAGA
CCTCAGCCTCCCAGTAGCTTAGACCACAGGTGCCGACCACCACACCAGGCTAiTTTTTGTATTTTTACTAGAGAATAGCGTTTCACCATCTTGG
CCCGGCTGCTTTCCAATTCCTGACCTCACGTGATTTGCCACCTCAGCTTCCCAAAGTGCTGGGATTACAGGTGTGAGCCACCACTACCCAGCAA
TTTTCTTTTAGCTTTGTTGATTATATCTTTCTCTTTAAAGAAGGTTTTAATGTTTATGTAGTAGATTTGTCGTCTCCTTTCTAGTTAGGATAGG
ACTTCCCCACACCAAGATTATAAAAATAACTGAAGCCAGGCACAGTGGTTTACACCTGTGACCTGTATCCCCAGGACTTTCGGAGGCTGAGGCC
AGAGGATTGCCTGAGCCCAGGAGATAGAGGCTGCAGTGAGCTGTGATTGAGCCACTGCCCTCCAGCCTGGGCGACAGAGTGAGACCCTGCCTCA
AAAAAATAATGATTATATTTTACATATTTTAAAATAAAAAATAATTGAAACATTTTATGGTTTTTTTTAATATGTAAATATGGTTGATCTAGAA
TTTACTTTTGTGCTTACAATATGAGGTAAGACTTTTTTTTTTTTTTTTTTTTGGAGACGAGAGTCTGTACTCTGTCACCCAGCCTGGAGTGCAG
TGGCACGATCTCAGCCCACTCCAACCTCCACCTCCCTGGTTCAAGCAATTCTCCTGCCTCAGCCTCCATGGTAGCTGGGATTACAGGCGCCTGC
CACCATGCCCAGCTAATTTTTGTATTTTCAGTAGAGACACGATTTCGCCATCTTGGCCAGGCTGGTCTCGAGCTCCTCACCTCAGGTGATCACC
TGCCTCAGCCTCCCAAAGTGCTGGGATTAGGGTGTGAACCACTGCACCCGGCCTTAGCTCTTATTTTCTATGTGGATAGCTGCCATCCTGGCCC
CGTTTATTTTAAGTTAACTCTCCACTGATTCACATTACCACCTTTGTCATTTTCTGATGTTTTTATATGCAGACACATGACTGTTTCTGAAATG
ATTCGTTCTGTTCCACTCAGCTGTTTGTCCACATGCTAGTAACCAAACTGTCTTAATTTCAGTAGTTTTAGTTTGTCTTTTTCTCTGGTATGAG
ACAAGTGCCTCTTATTTTTTTCTTTTTTCCCAAATTTTTTCAGCCATTCTTGGGCACTTATTTGTTCCAATGAACTTTAAAATGAACTTCTCAA
GAGCCCCCTCTCAAATCTCCTTGGGATCTTTCTAGAAATTGCATTAAATGCAGAAAATAATTTGGGTAGTAAATTTTGATCAGTAGTATGTAAT
TATCTTGAGCCCCATTTTTTTAAATACACTTTTTCCATCTCCTTTGGCAAGAGAACTTTGGTAAGCTGTTTACTGAATTTATTAAAATTCGTCC
TTTCTAAATATAGACCATCTATTCCTAGGCCCTTGGACTGGACAGTGCCAATGAGAATGGCTTGTATAGGAGGGGTGATAGCCCAGCTGCTCAT
GAACGAGTTTGAGTCAGCCAAGGGTGACTTTGAGAAGTGCTGGAAGTAACCCCCAGATTTACGCTGCAGTAGACTGCAGATCTCCATGTGCCAG
AAAAAGGCCAAGGAGCACAACGAGCGGGACCGCAGGATATACGCCAACATGTTCCCGGATGTTTGCAGAGCAGGATGCCTAGGTGTGTGAGAGC
CTGATGACTGCAGGTGGATCAAGAGCCTGACCATGCCCCTTTGATTTTAGGGGACAGGAACAGCTAAACAACCCAAGAGGCTCAAGAGCAGGGA
AAACGATTTCCAATCTAATCTGAGGCTGCTTTGGATCCAAGGTCGAGTACCGCAGTTCCAGTCACCAAACGCACACGTCATCAGAGATGAGAAA
AACTAAATTCCCTTCAGATGTATTAGATTGGTTCAAAAGTAATTGTGGTTTTTACCATTACTTTCAATGGCAAAAACAGCAATTTTGCACCAAC
TCCCCCCGCCCGCCCCCCGGGCCCCTGCCCGAGTCAGAGTCTCACTCTGTCACCCAGACTGTAATGCAGTCGTGCTAATGCGACTCACTGCAGC
CTTGACTTCTCTGGATCAAGTGATCCTCCCACCTCAGCCTCCTGAGTAGCTAGGACAAGCACACTACCACACTGGGCTACTTTTTTTAAATTTT
TTGTAAAGATAGGGTTTCACTTTGTTGTCTAGGCTGGTCTCGAACCCCTGGCTTCGATGGGTTCCTCCTGCTTTGGTCTCCCATGTGCTGGGAT
TATAGACATGAGCCACCATGCCCAGCCCTTTTGTCATTTTTGAAAAATTCTAAACCCATCAGGATTTTTTTTTCAGTGTAGACTTACGATTTTC
CCAGGAAATGGGATCTTATAGGAGCCCCATCATCACACCTGTTATCTTGAGGGTCGGCAGCCAGCCTGAAATATGTGGCAGGAGAGGTCTGTCT
GCAGAGAGGGCGGCCTCCCTACAGCATAGTTGGCATTCTGGTCCTGTCCCCAGTCTCCAGGAATTGCCCAGACTTCATTATCCACAGCTGTCCC
ATGTGTCAACCAAAGAAGGTGAAGCTCCCACTCTAACTATTGGTAGCACAGCCCTGACCTACCCACCTAGATATCCTAGAGAATGCTCATTGCA
ATACCTCGTGTTTGGCACCTGATACACAGTCTCGAGGACCCAGQAATTATTTCACTTCTTTATTCTCTTACCTTCTTGATTGCTTATTTCTTCC
TTAGGAAGAGGCCAATAAAGCAATGGGCAAGAAGACTTCAGAAGGGGTCACTAATGAAAAAGGAACAGACAGTCAAGCAATGGAAGAAGAGAAA
CCTGAGGGCCACGTATGACGCCACGCCAAGGAGGGAAGAGWCCCAGTGAACTCGGCCCCTCCTCAATCGGCTTTCCCCCAACTCAGGACAGAAC
AGTGTTTAATGTAAAGTTTGTTATAGTCTATGTGATTCTGGAAGCAAATGGCAAAACCAGTAGCTTCCAATGAGCAGCCCCCCTGCTGCTGCCC
GGAGGGTTCACTGAGGGGTGCCACGGGACCACTCCAGGTGGAACAAACAGAAATGACTGTGGTGTGGAGGGAGTGAGCCAGCAGCTTAAGTCCA
GCTCATTTCAGTTTCTATC~CCTTCAAGTATCCAATTCAGGGTCCCTGGAGATCATCCTAACAATGTGGGGCTGTTAGGTTTTACCTTTGAAAC
TTTCATAGCACTGCAGAAACCTTTAAAAAAAAAATGCTTCATGAATTTCTCCTTTCCTACAGTTGGGTAGGGTACGGGAGCAGGATTAGGCTTT
TGTTTTTTAAATGACTGAAGTGCTATAAATGTAGTCTGTTGCATTTTTAACCAACAGAACCCACAGTAGAGGGGTCTCATGTCTCCCCAGTTCC
ACAGCAGTGTCACAGACGTGAAAGCCAGAACCTCAGAGGCCACTTGCTTGCTGACTTAGCCTCCTCCCAAAGTCCCCCTCCTCAGCCAGCCTCC
TTGTGAGAGTGGCTTTCTACCACACACAGCCTGTCCCTGGCGGAGTAATTCTCTCATTCCTAAAACACCCTTCAGCAATCATTATGAGCACATG
AGAGTTTCTGGATTAGCTTTTCCTATTTTCGATGAAGTTCTGAGATACTCAAATGTGAAAAGAGCAATCAGAATTGTGCTTTTTCTCCCCTCCT
CTATTCCTTTTAGGGAATAATATTCAATACACAGTACTTCCTCCCAGCATTGCTACTGCTCAGCTTCTTCTTTCATTCTAATCCTTGCTATTAA
GAATTTAAGACTTGTGCTTACAATATTTTTGACCTGGAGTGGATCTATTTACATAGTCATTTAGGATCCATGCAGCTTTTTTTGTCTTTTTAAG
ATTATTGGCTCATAAGCATATGTATACTGGTTTATGGAACTTTATTTACACTCCTCTATCATGCAAAAAAATTTTGACTTTTTAGTACTATGCT
TAATTTTTAAAAACAAAATCTGTAGGGTTGACAAATAAATAGTTGCTCTTCTACACTAGGGGTTTCACCTCCAGGTTTGACACGCAGTTGCTCG
CTTTTCCTGCCCTGTCAAGCTTCTCTGTTCTGGCGTGAGTTGTGAAAGAGTTGAAGACAGCTTCCCATGCCGGTACACAGCCAGTAGCCTAATT
CTCCAGTACTTGAGCTGACCATTGAACTAGGGCAGTCTTAATGTGTACATGTAGTTGAATTTCAGTCCTTACGGGTATCAACAGATTGAGCATG
GCTCTCTATTCCCTCAGCCTAAGAACACTCATGGGAATGCATTTGGCAACCCAACGAACCATTTGCTTAAACCTGGAACATCTCACCTTTTTTA
AATCCTAAAAAACACTGGCAGTTATATTTTAAATTAGTTTTTATTTTTATGATGGTTTTATCAAAAGACTTTTATTATTAGATTGGGACCCCCT
TCAAACCTAAAAATCAAGTTATTTCCTTTTATAATACTTTTCTTCCCCATGGAACAAATGGGATCAATTTGTGAGTTTTTTCCTTTAATGATAA
CTAAATCCCCTCTAATTTCTCATTTATGCTTTTGTCTTTTTTATGAAATATTTCTTTTAAAAGCCCCAGTCTCACCTACGAAATATGAAGAGCA
AAAGCTGATTTTGCTTACTTCCTAAACTGTTGCGAAAGCTCTGTAGACCATGGTTCCAGTGAGGCCAAGATTGATTTGATACTAAATGTCGGCC
ACCTAGCTTTTTGCAGATAACAAACAAGAAAGCTATTCGAGACTCAGATGATGCCAGCTGTCTCCCACGTGTGTATTATGGTTACAGTAGGGGG
AACTGGCAAAAGTGTGTGTGGGGAGGGGAGGGTGTCTGAGTGGTTCTGAGCAATAACTACACGGTGCCCATTACCACTCCGGATAGGACACTTC
ACGTATTCTTCTATCAAATTCAATAATCTTAAACAATTTCTGTAGAAGTCCACAGACAPCTTTCAACCACCTTTTAGCCTGCATATCGATTGCC
AAGTCAGCATATGAGGAATTAAAGACATTGTTTTTTAAAAAAAAAAATCATTTAGATGCACTTTTTTGTGTGTTCTTTAAATAAATCCAAAAAA
AATGTGACTTCCCATTGTTTTTGGTTTTGCTTAAGTGATTTTCAGCGAAAGCTATATTGGTTAAAAAACCATGGTGGGGGCCAACTTGATAGAC
TTACACATCAGAGACGTCAGGCGTAGTGGCTCATCCCTGTAATCCCAGCACTTTGGGAGGCCAAGGCAGGTCCATCACCTGAGGTCAGGAGTTT
GAGACCACCCTGGCCAATGTCCTAAAACCCTGTCTCTACTAAAAATACAAAAATTAGCCAGGTGTGGTCGTGCACACCTGTAATCCCAGCTACT
CAGGAGGCTGAGGCAGGACAATCACTTCAACCTGGGAAGTGGAAGTTGCAGTGAGCCAAGATTGTGCCACTGCACTCTGGCCTGGGTGACAGTG
AGACTTGGTCTCAAAAAAAAAAAAAAAAAAAAAAAATCAGAAAATCGGCAATGGACAGTATCATCCTACACTTTGCTCTAAGGTGAACCTGAAA
CCAACATTGGGGTGAAAGTGGCAGAACAATGCATCATTGCTAAAAAGAACCAAGTAATGGGAAAAATAAAAAGGTTTTGTTTTTTCATTAGCCA
GCGGATATTTCACCTTTTTAAGCTGATTTTAAGGTAGGCTCGACTTGCAAAATAAAATTTAATTGTGCCCTTTAATAAATATATGAAACATAAG
TAAGGCTATCTAAGGTGGTGGTCCATATTGAGCATGCATTTTCTTTCTTAAGTAGCATATATCATCAATACTCATCATCACAGACTCTCAAGGC
CTAAGGAGATGTCACATATACTGCAAATCCTCATTTTCACACTGCAAAAGAAGGTGGCACAGTGCTGGAAGGTACTTAGTAGGACCTCTGAGGT
CTACAGACAGGTTGATGCAGGTTCTAGGCACCCTTTATTCCCTCTGAATTCTTGGCTTCATCTCTGGATTTGAATTATATTTCTCCCTCCTCCC
TGTTAAATTATTTGGTAATGAAATAAATTCCTAGCTACGTCCGGTTGCTTATGCTTATAACCCCAGCACTTTGGCAGGCCGAGGTGGGAACCTT
GCTTGAGCCCAGGAGTTCGAGACCAGCCTGGGCAACATGGCAAGACCCTGTCTCTACAAAAAGTACAAAAATTAGACAGACATGATGGTATGCA
GCTTTAGGCCCAGTTACAGGCTGAGGTGGGAGGATCACTTGAGCCCTGGGAGGTTGAGGCTGCAGTGAGCCATGATCATGCCACTGCACTCCAG
CCTGGCTGACAGAGCAAGACCCTATCTCAACCATAATAATTCCCTTTATTTGCGTGCTTTTTTTTTGTAAGAAAAATAATGATCACCCTAGGGA
TCTTGAGGATGAGCCAGAGAAGATTCCTGGCTGTAAATGTGTGATCTCAGAATTCAGAGGCAGCTTGCACAACCTCCAAAAACGAGGACTGCCA
GTGAACGGCTTTGTCTATACTTACTTGGCCTTAGCAGCACACTGAACTCCACTTTTAGTATGGTACATCTGAAGCCTGTATTCTCTGGGGCTGG
AGACTATCTCCAGTATTGGTGACAAAACGCATTGAATTTGAGCCAGTTAGTCAAAATCCTGAAACTCAGAATTCCCCAACCCAGCTGTGTATTT
TTTCCATCAGGTGGCTACTTATGCATCTGAATGACAGTGACTGACGAAGTACATAAACGACATTCTCTGATGGCTTGAAAAAAGTTTTGGTAGA
CCTTCAGTACTTCCCTTCAGATTTTTCTCTACTACATGTCTGACATGGAGATTTCTGTGGGAGGAGAAAGGCACTATCTGTAATTGCCCAGATC
TCCACGTGTGACCTTAATAATACACGGCAAGAAAGAGACAGCGTTAAATACAACTCTTAAGATTTTTGACAGCATCTTGTTTGGGTAACTGCAG
CCTATAGTTAGAAAAATTAAAGCACAGTTTATAAGGCAGGGATTCTTTGTCTTCTAAAGATGAGGGAACCTTGGAGAAATCAAGATATGGGGAG
GACCCAGAATGGTGATGCAGCCTGCCCATGGCCGTCAGGTCACCTGATGCCCTTGCACCATTTTAAAATAGGTCTTTTTCTAGTATGATGAGTG
ATGCTCTTGGTGTATGGAAGGAGATGTTGGTACTTCCTTAGAATTCCCACATTCGGTGGAACTGAAATGGCAGCAGAGCCATGGGCCTCATAGT
CTACTTTGGAATAATCTATGGTGGGAAGCTTGCCAACGTGTTAAGCAGAGGCATCTTTTTAAAATACACTTTAGGGGCCAGGGGATGGTGGCTC
ACACCTGTAATCCCAGCACTTTGAGAGGCTGAGGCAGGAGGATTGCTTGAGGCCAGGAGTTCAAGACCAGCCTGGGCAACATAGTGAGACCCAA
TCACTACCACACACACACACACAAATTAATTAGCTACACATGCTGGTGCATGCCTATAGTCCCAGCTACTTGAGAGGCTGAGGTGAGAGGATAC
TTGAACCCATGAGTCGTACGCTTCAGTCAGCTATGATCACGCCACTGCACTCCAGCCTGGCCAACAGAGTGAGACCTTCTCTCTAAATTTAGAA
AAAAAAATCTGGCTGCTACATTGTTTCCACCTCTCAAATCTCCACAAGGTCATTGGTCCTTCCTAACCCAGAACTAAGCAGGGAAGGAATTCTG
GGACATGTGTTTCCAACTTGGCTTAGTTGACACATTGCAAATACAACATCTGGCTAAGGCTTTCTTTTTTGTTTTTGTTTTTGAGAGGGAGTTT
TGTTCTTGTTGCCCAGGCTGGAGTGCAGTGGCACAATCTCGGCTCACTGCAACCTCTGCCTCCAGTATTCAAGTGATTCTCCTGCCTCAGCCTC
CTGAGTAGCTGGGATTACAGGGTCCGCCACCACACCCAGCTAATTTTTTGTATTTTTAGTAGAGATGGGGTTTCACCATGTTGGCCAGGCTGGT
CTCCAGCTCCTGACCTCACGTGATCAACTTGCGTCAGCCTCCCAAAATGCTGGGATTACAGCCGTGAGCCACCGGGTCCGGTCTGTATCTAAGC
CTTTCTTGAGGTATTAAATATTTCCAGCCTGGGTCTGCTCTGGGATAATGTGTTTTCATAAGATAACTCCTTATTGTAGGCATTGTTCTTGGCT
TAAAGTTATCTCCTTAAGCTTCTGACTAGGGAAATAAGTGGTAAGAATAAACAGTTGATGAGTATTTGGGGATGCAATGACTAAATTCTAGTTA
CTCTAGTTCTAGTZAATGACTTCATGAGTTTGTATTGTATCTGCTCACAACCGTGTTTCCACACTAAGGACTCCTGAAATTTTTTTGTCTTAAA
ACTTAAACCCGCTGGGTGTCCTGGCTCACACCTGTAATCCCAGCACTTTGGGAGGCCGAGGAGGGCGGATCACGAGGTCAGGAGATCAAGACCA
TCCTGACTAACATGGTGAAACCCCATCTCTACTGAAAATATGAAAAATTCGCCAGGCTTGGTGGTGGGCGCCCATAATCCCAGCTACTCAGGAG
GCTGAGGCAAGAGAATGACGCGAACCCAGGAGGCGGAGCTTGCAGTGAGCTGAGATCGCCCCAGTGCACTCCAGCCTGGGCGACAGAGCGAGAC
TCTGTCTCAAAAAATAAAAATAAAAAACTTAAACCCATGTGTATCCTATTGCCCTCTGTGGCTACCAGAGTCTTCCTAACATGAAACCAGCCTT
TACCTGTAGTTAACAAAATGTGATTCCCCTTCTCCCACTCAGGGATCAGGAACACTGCCTTACTCATCACCATCCTCAGCACCCAGTCCATCAT
CCCCAAACTAGAACATAGTAGATACTCGGTAACCTGGCTTTGTCCCTCCTCTGAAGCCAGCCTCTTCTCTGTATCCCAGTGGCCCTTCACTCCT
GTTACCTGAGATGTGGAGGCCAAAGCCCCATGATACTGTCGTTGTCCCTGCAGCCTGATTTTGTGTCTTGAATTCTTTTCCAAGTGCTGAACCA
GCAGCAACTTGAATTTTGGGAGCCCATAACTGTAAAATGGGTCCATCGTCTTCACGCTACATTCATCTTATATCTATTTTTAGTCACAATTGAT
AACTCACAGTTCACCAAATACGAAGATCAAATATAAAATATTTTCCTTAAAAGTATTCThAGACCACCCCGCCCAGTCTGGTGCCCTGGTCAGC
CAGCCCCCAAACATTTACGGCCCATCTCTTGTGAACTGGGTACTGTTCCACTGACGTGAAGQATGGGCCCCCCTGGCCTGAGCGACTCGCAGAC
AAGTGAAAAATGAGGCATGCAGATGGCTGCATCATCACAGAGCATCTGCCATCTGCTACTGACATCTACAAAATAGGGAACAGCAGGAAACTCA
AGCCTGCCTCTCCAGCTCTCCCCACTCATTCAGAGCCACACTGGAGACCGAGTCAAGCCAGGGCCCACCCCACTGCTGACATCCAGCCCAGTCC
TACTGGGCAAGCCCTTCCTCAGCACTGCCCCCAGCTGGCCACTGTTCCACCCCTGTGCTTCTCTCCAAGCATCCGGGCTGCAAAAAGTGCCCAG
CCCTACCCTGAGCTATACCAGCCCTCTTGCCCTGGGCCCAGCCAGGATGGTTTCTCCTGGTGGCAGGATGTTAATCTCTTTGGCACACCAGGCA
GAAGTCTAGGAGCAGAGAGCTATGTCCTTAGGTCTGTTTTTCAGTTACCTGCCTACTGGAATACAGCTGCAAGAAAACTGTGTCCTTTATTTAC
GAGACAACTGTACAGATACACCATGCCTAACTTTGCTTACTGCATGGTCACTAGGATGTACTGAGTTTCAAGGAAATATGTTTTTGCTACATTT
TAATTAGCACTACCCCCCTTAGGTGAAATTTGGGCCAGGTGCAGTGGCTCACACCTGTCATCGTAGCACTTTGGGAGGCCAAGGCAGGAGGATC
ACTTGAAGCCAGGAGTTTGAGACCAGCCTAGGAAACAAAGCAAGACCCTGTCTCTACGAAAAAATTTTAAAATCAGCCGGGCGTGATGGCGCAT
GCCTGPAGPCCCTCCTACTCAGATGGATGAGGTGGGACGACTGCTTGAGCTCAGGACGTCCAACCTGCAGTGACCTCTGATCATGCCACTGCAC
TCCAGCCTGGGTGACAGAGTGAGACCCTGTCTCAAAATAAATAAATAAATAGGCTGGGCTCGGTGGCTCTCACCTGTAATCCCAACACTTTGGG
AGGCCGATGGGGGCGGATCATGAGGTCAGGAGATTGAGACCATCCTGGCTAACACGGTGAAACCCCGTCTCTACTAAAAATACAAAAAATTACC
CGGGCGTGGTGGTGGCTGCCTGTAGTCCCAGACACTCGGGAGGCTGAGGCAGGAGAATGGCATGAACCTGGGAGGCAGAACTTGCAGTGAGCTG
AGATTGTGCCATTGCACTCCAGCCTGGGCGATAGAGTGAGACTCCGTCTCAAAAAATAATAATAACTAAGTAAATAAACTTCCTCACTGTTCTG
CGAACCCTCACCCCTTTGTCACCAACACACTTTCCCTGCAGAGAAAGCTACACTGCTTGACTCAGTTGGTAAATGCTCCTCTCTCTCTCCACTC
TGTCTCTCGCAAGGGCACGCAAGGCTTTCTATATGGGCTTCTAAGCTTTCTCTGAACCCAGCTGTCTGGTTGACAAGGAGCAGATTGTAATAAG
ACAATACAGTGACGTTTGCACCCTTCTACCCTTCAGCGGPTACCAAATGTGACAGTAGGTAACTAAACCAAGCCACCTTCAGCCTGCAAGTCGG
GGCCGACAGAACCAAGTTATAACCTGAGATAACCCCAGTTGGCWGATGATTGCCGAGACCAGACTTCCTACAGACAGTGCTGCCCACTTCTGCG
GTGATAACTGCAAAACAGGGCCCCTCATAATGTCCTCATCAGGACCTCCTCAATCTGAGGAACACACAGGCCTCCAATGTTTGCCAAGAGCACT
CGTTTGGGTCCAGCTCTGTGTTGGGATCTCTTGTTTCCTCACCTGTGAAATAACAATGGCTAATATTTATACAACTCTTTTAATTATAGTAGAA
AACACATACCATGAAATTTATCATCTTAACAATTTTTCAGGTTTTTTTTTTCTTTTTTGAGACAGAGTCTCTCGTTCTGTTGCCCAGGCTGCAG
TGCAGTGGCACAATCTTGGCTCACTGCAACCTCCACCTCCCCCGTTCAAGTAATTCTTGTTCCTCACCCTCCCAAGTAACTGGCCATGGGCCAT
CATGCCCGGCTTTTTTCTTATTTTTAGTAGAGACGGGGTTTCACCATGTTGGCCAGGCTAGTGTTAAATACAGTAAGAAGTTCTTCTCAAAGGT
TTAGCTTGTTTAAGTTTCCTTGTCCTCTGTTCCCTCCTTTCAAGGCCAGACTTCCTTACTCTCTGTGTCCCCCTGCCCTGGTAAACAACCTTCC
CACCAGTCCTTATCTATACAGCCCACATTCCACATCTGCTACCCACTCTGTGAATTACCCCTCCCGTCACAACGACCCTCCCCGCAGAAACTGT
TCCTCCCACCCCCCACCAGTGTAACCGCATTCTTGCACTTTTCAAGTTAGCCAACCGGGTTCAGCTTGGATTGGTCCAGCTCCAGCCAATGGAG
ACAGGACACAGTAGCGGGCACAAGCTGTGTTAGGAATAAAAACCCCTTCCCTCCTTTGTTTGGCGTGCTCTCGTGGCGACCAGACCTACAAGAA
GCACCCTTCTCCAGAACCAAATTTGCCTTGCTGAGACATCCTTTCTCTCAGTGCTGCTTCTTCTTTCCAGCACTCAGCATTTGTTTCCAACAAA
TTTGGTGGCCCATATGCGGACCTCATTCTCCTCCGGGAAAGGGTCTCTGATCACTTCTCATGAGGAGACGCATCCCGCTAACTCATTGCAGTGG
CCTCAAGGGTAAAGAATCGGGACCCACCCAGTGCAACCAATAAACCCAGACTCTTAGCAACACAGGAAGAAAAGGCCTACAGTTACTGTGGTGA
CCAGGTAACGCTATGCACAGACCAAGCTAAGAAAAACTGTQGGGGCGCTGAAGTACTTCCTTGGTGGTAGGGACAGTTTGGAGGTTAAAAGTGT
GTAAATAAGATGCACAATTAAGTGCAAAGCGAGTGTCGAGTAAAGTAAAGGAGTGCAAGAAATCTCCAGTGGGGGAGCTGAGCCTCCAGGGAAA
GGGAGCAAAAAAAAAACAATCTCTAGTAAAAGAGGTTGACCCAGGAGGCCGAGGCGGGTGAATCACGAGGTCAGGAGATCCAGACCGTCCTGCC
TAACACAGCGAAACCCCATCTCTACTAAAAATACAAAAAAAAAAAAAAAATTAGCCGGGCGTGATGGCGGGCGCCTGTAGTCCCAGCTACTCGG
GAGGCTGACGCCAGGGAATGGCGTGAACCTGGGAGGTGGAGCTTGCAGTGAGCCAAGATCGTGCCACTGCACTCCAGCCTGGGTGACAAAGCGA
GACTCTGTGTCAAAAAAAAAAAAAAAAAAAGAGGTTGAGCCCCACACACACTCAGGTTGAGCCTTCAGGGAAAAGGGTGCAAGAAATCTCTAAT
AGGAGAGACTGAGCCCCCATTAACTTCCAGGATGGGAAATACCCCTACTAAAACAAGAACTACAAAGGACCAAACATAAGAAAAAGCAACAGAT
GATAAAATATTGTTGTTTTTTTTGAACCAAAGAAACTATTCTTAAACCTTTAGTTTTCTGGCCAAGTTTTGGGTCAAATAAGGATTAGATTTGT
CAACTTTTAATAAAGTATGTAAATAACAAAACTCCTGTCTCCCAGGAAGAAATAGACTATGCCCTGCACTGGCGGCACCCCCCTGTCCTCTACC
CCCTAAAAACTGCAGGAAAAAAAGCCAGAAAATACCTCCCCTAAAGAAAGTAAAATCCCTAACCCCAAACAGTCCACTAGCACATGGGATTCAA
AATTTCCCTTTCCCCTCCTCCTCAAAAAACTCTACCCCAACCCTTTTTCCCTTAACGCAACTGCCTCTAGGACGAGGCCAAATTGGCTTTGTAA
ATGCCCCTTAACTAGTTCAGAGGTCAGAAACCTAAGGAAGAAACTTAAACCACTATTAGATGATCTTTTTAGGGTTGTGGATCAAATTAACCGA
TTTTTAGGACCACAAGTATACACTTGGGCTGAGCTAATGTCCATCTTAAGTATTCTCTTTTCGGGAGAGGAAAGAACCATGATCCGCAGGCCTG
CTATGATAGTCTGAGAGCGCAAACACCTTCCCAGTCAAAATATCCCWGCAGCAGAACAAAAATCGCCGGCCCAAAACCCCCAATCGGATAATAA
CAATGCAGCCCACCAAGAAAACATGAAAGGCCTTAGGAAAATGATAGTTAAAGGGATTCGGGAATCAGTGCCTCAAACCCAAAATATTTCCCGA
GCATTTAATATCCAGCAACGAAAAAACAAAGGGCCCACGGAGTTTTTAAACAGACTCAAGCAACAGGTAAGAAAATATACAGGCTTAAACATAA
AGGATCCCCTTGGACAGGGGATGTTAAAGCTCCATTTGGTTACTAGTAGCCAAATATCACAAAGAAATTACAAAAACAGAGAACTGGAAAGATC
AGCCTATAGAGGGAATGGAGGCAGTCAGGGAGAGAGAGAAAGAGAGGCAAAGAGAGAAGATGGACAGGCAAGGAGAGACAGA
HUMAN SEQUENCE—mRNA
GGGCCGGCTCGCGGGCGCTGCCAGTCTCCGGCGGCGGTGTCCGGCGCGCCCCCGGCCTGCTGGGCGGCCTGAAGGGTTAGCGGACCACCGGCAA
GGCGGAGAGTGACGGAGTCGGCGAGCCCCCGCGGCGACAGGTTCTCTACTTAAAAGACAATGACTACTGATGAAGGTGCCAAGAACAATGAAGA
AAGCCCCACAGCCACTGTTGCTGAGCAGGGAGAGGATATTACCTCCAAAAAAGACAGGGGAGTATTAAAGATTGTCAAAAGAGTGGGGAATGGT
GAGGAAACGCCGATGATTGGAGACAAACTTTATGTCCATTACAAAGGAAAATTGTCAAATGGAAAGAAGTTTGATTCCAGTCATGATAGAAATG
AACCATTTCTCTTTAGTCTTGGCAAAGGCCAAGTCATCAACGCATGGGACATTGGGGTGGCTACCATGAAGAAAGGAGAGATATGCCATTTACT
GTGCAAACCAGAATATCCATATGGCTCGGCTGGCAGTCTCCCTAAAATTCCCTCGAATGCAACTCTCTTTTTTGAGATTGAGCTCCTTGATTTC
AAAGGAGAGGATTGGAATTTGAGATGGAGGCATTATCCGGAGACCAAACGGAAAGGAGAGGCATATTCAAATCCAAACGAAGGAGCAACAGTAG
AAATCCACCTGGAAGGCCGCTGTGGTGGAAGGATGTTTGACTGCAGAGATGTGGCATTCACTGTGGGCGAAGGAGAAGACCACGACATTCCAAT
TGGAATTGACAAAGCTCTGGAGAAAATGCAGCGGGAAGAACAATGTATTTTATATCTTGGACCAAGATATGGTTTTGGAGAGGCAGGGAACCCT
AAATTTGCCATTGAACCTAATGCTGAGCTTATATATGAAGTTACACTTAAGAGCTTCGAAAAGGCCAAAGAATCCTGGGAGATGGATACCAAAG
AAAAATTGGAGCAGGCTGCCATTGTCAAAGAGAAGGGAACCGTATACTTCAAGGGAGGCAAATACATGCAGGCGGTGATTCAGTATGGGAAGAT
AGTGTCCTGGTTAGAGATGGAATATGGTTTATCAGAAAAGCAATCGAAAGCTTCTGAATCATTTCTCCTTGCTGCCTTTCTCAACCTGGCCATG
TGCPACCTGAAGCTTAGAGAATACACCAAAGCTGTTGAATGCTGTGACAAGGCCCTTGGACTGGACAGTGCCAATCAGAAAGGCTTGTATAGGA
GGGGTGAAGCCCAGCTGCTCATGAACGAGTTTGAGTCAGCCAAGGGTGACTTTGAGAAAGTGCTGGAAGTAAACCCCCAGAATAAGGCTGCAAG
ACTGCAGATCTCCATGTGCCAGAAAAAGGCCAAGGAGCACAACGAGCGGGACCGCAGGATATACGCCAACATGTTCAAGAAGTTTGCAGAGCAG
GATGCCAAGGAAGAGGCCAATAAAGCAATGGGCAAGAAGACTTCAGAAGGGGTCACTAATGAAAAAGGAACAGACAGTCAAGCAATGGAAGAAG
AGAAACCTGAGGGCCACGTATGACGCCACGCCAAGGAGCGAAGAGTCCCAGTGAACTCGGCCCCTCCTCAATGGGCTTTCCCCCAACTCAGGAC
AGAACAGTGTTTAATGTAAAGTTTGTTATAGTCTATGTGATTCTGGAAGCAAATGGCAAAACCAGTAGCTTCCCAAAAACAGCCCCCCTGCTGC
TG
HUMAN SEQUENCE—CODING
ATGACTACTGATGAAGGTGCCAAGAACAATGAAGAAACCCCCACAGCCACTGTTGCTGAGCAGGGAGAGGATATTACCTCCAAAAAAGACAGGG
GAGTATTAAAGATTGTCAAAAGAGTGGGGAATGGTGAGGAAACGCCGATGATTGGAGACAAAGTTTATGTCCATTACAAAGGAAAATTGTCAAA
TGGAAAGAAGTTTGATTCCAGTCATGATAGAAATGAACCATTTGTCTTTAGTCTTGGCAAAGGCCAAGTCATCAAGGCATGGGACATTGGGGTC
GCTACCATGAAGAAAGGAGAGATATGCCATTTACTGTCCAAACCAGAATATGCATATGGCTCGCCTGGCAGTCTCCCTAAAGTTCCCTCGAATG
CAACTCTCTTTTTTGAGATTGAGCTCCTTGATTTCAAAGGAGAGGATTTATTTGAAZATGGAGGCATTATCCGGAGAACCAAACGGAAAGGAGA
GGGATATTCAAATCCAAACGAAGGAGCAACAGTAGAAATCCACCTGGAAGGCCGCTGTGGTGGAAGGATCTTTGACTGCAGAGATGTGGCATTC
ACTGTGGGCGAAGGAGAAGACCACGACATTCCAATTGGAATTGACAAAGCTCTGGAGAAAATGCAGCGGGAAGAACAATGTATTTTATATCTTG
GACCAAGATATGGTTTTGGAGAGGCAGGGAAGCCTAAATTTGGCATTGAACCTAATGCTGAGCTTATATATGAAGTTACACTTAAGAGCTTCGA
AAAGGCCAAAGAATCCTGGGAGATGGATACCAAAGAAAAATTGGAGCAGGCTCCCATTGTCAAAGAQAACGGAACCGTATACTTCAAGGGAGGC
AAATACATGCAGGCGGTGATTCAGTATGGGAAGATAGTGTCCTGGTTAGAGATGGAATATGGTTTATCAGAAAAGGAATCGAAAGCTTCTGAAT
CATTTCTCCTTGCTGCCTTTCTGAACCTGGCCATGTGCTACCTGAAGCTTAGAGAATACACCAAAGCTGTTGAATGCTGTGACAAGGCCCTTGG
ACTGGACAGTGCCAATCAGAAAGGCTTGTATAGGAGGGGTGAAGCCCAGCTGCTCATGAACGAGTTTGAGTCAGCCAAGCCTGACTTTGAGAAA
GTGCTGGAAGTAAACCCCCAGAATAAGGCTGCAAGACTGCAGATCTCCATGTGCCAGAAAAAGGCCAAGGAGCACAACGAGCGGGACCGCAGGA
TATACGCCAACATGTTCAAGAAGTTTGCAGAGCAGGATGCCAAGGAAGAGGCCAATAAAGCAATGGGCAAGAAGACTTCAGAAGGGGTCACTAA
TGAAAAAGGAACAGACAGTCAAGCAATGGAAGAAGAGAAACCTGAGGGCCACGTATGA

TABLE 15

MOUSE NOMENCLATURE

| | |
|---|---|
| ICSGNM | Rel |
| Celera | mCG8770 |

HUMAN NOMENCLATURE

| | |
|---|---|
| HGNC | REL |
| Celera | hCG15154 |

MOUSE SEQUENCE - GENOMIC
GTCCAAAAAAAGACTCCTTTTTCATATTAATAAACCATAGACAATAACAACAGTTATTAAACTATCAGGTAAGAATTGTATTCATAATTGTTCT
GGTTTAAGCATTCAAGAGATAGATGGAAAGGTGGAAGCACTAGAAGACTCGATGGTCATGCATTACAAGCAGCAGCCCAATGGGCTGGAGCGCC
AGAAGAAGACATTTGAGCCACAGTCTCTAGAAACTGTTCTCAGGATGAACTTAGAAAAGCTTTGAGCACAAGCAGTGCTGACTGAGGTGTATAT
ACCAAAGTGTCATACGCTATTTGACAGAAGGAAGGTCTTCTGTACTAAATAAAACAGGAGAGAAGAGACCATGCTCCACTGCCTTATGCCCATT
CAGGTCTAACCACTTTAACTGGAATGGCATATCCATGATCCCGCTAGAAAGCAGACACTAATGCTGGGACATAATCTCTCTCCATACTTAGAAC
AATGTTTAGCATGAAGTGTGTACTGAATCATGAATTGATTCAACAAACATTTATAGCTTGACTTTTATTTATATATTCATAATCCCATGACAAC
TTACAGAATTAATATGACTATGACAAACATGTATTCATTTTATTCAGTAAATCTAGAGATATAAAAGTACACTTTTATCAATTGTACTATTAGT
TCAAGCAAGCACCAAATAACACTGAAGGTGTACCTAAACACAACATGGGTATTCCCCATACCTAAGAGGAGGCAGGAGGATTGAGACTTTGAGG
TCAGCCTGAGATATACAATGAGACATTGGCTCAAATAAATAAATAAATACATAAATAAATATATTTACTATGAAAATGAAAATACTACCTGGAA
TATGAAAAAATAAAATTCAGTGTTAGATAAAAGTTATACTTGTCTTCAAGTTACATTAGATATCTAAATATAGGTCTTTCTTTTTTAGTCAATA
ATTTACAAAGCCCAGATTCCAGTTCATATTTTTCCATTCTCCCAATCTTCTAACTCCATGGTCATTCAGAATCTTTAACCTGAGAGGATATGGC
AGTCTCAGAATGTTCCCAGTTTCCTAGAACAGCTGCTTTCCACCCAGTCCTATTTCTGAGCTCAGTATAAAATCCCTGTAGCACAATGGCAAAA
TGCAAAACAAAAAAATATGTGCATCCTACATCCGCCATTTAGCGAGACACCCCCTTGCTTGATGCTTCCTCACTACTGAGGTTGTCAGGAAGTG
TAAACATTTCCCTAGAAACTGGAGGGAAAAAGAAAAAGTCCACTTGTGTTAAAGATTCTATGTCCAGTTAGAGTGTTACAGAAGAGACAGATTA
TCTTGGGCTATTTTTATTCTTCTTAAATATATATAAAATTAGCCGGGCAGTGGTGGAGCACAAAGTTAATTCCAGCACTCAGGAGGCAGAGGCA
GGCAGATCAGAGTTCAAGGCCAGCCTGGTCTACAGAATGCGTTCTGGGACAGCCAAGGTTACACAGAGAAACCCTGACTGGATAAACAAATGTA
TATATAATTCATTTTCCACGCCTGTGTCCAACCTAATATCCTCATAACTGTCTAAAACTTTAAAATGCAGTTTTAACAGACTTCTAATCTAAGA
AGACCCTCAGATAGCACCTGAGGCTTGCACCTCCCACTCTGTAACCAACAGACCAGATACAGATGAAGTACCTAAGGGTGTTCCGTGTCCTCAC
TTGTGAGATATGAGCTGGAATGAGTGGAGTCAGGAGTTCAGGTATGGCGGCACATCCTGCCCCACAAGTTCAACATTGGTTTGGGCCATAATGA
GTTCAAAGGTTAGTTACAGCAAGTCTCTGTCCAGGGAGGTCTGACAGATGGATCCCACGTGACCTCAGATGTCATTCCTGTTACAGAAATGAAG
TCACCCTGTGTCACACTTACAAGCGCTGCTATATGCACAAAACTGGCTTCGTCCTCTAGGCCTTACCTAGGATAAGCGTACAGACCGAAAGCCA
GAAGTTTCTGGATGGTTTACGACCTGCCTTTAGGAGTTCTCATGTACTCAAATTTAGTTTCTCTGGGACTTCACTAACCCAATGTTTCAGTTCC
TTTTAGCCTAGCTGAGTGGGGCCGCTTTCTCAAAGCTGTTTCTCACCTTGGGGACTGTCCCTCTTTCCTGCAGCTGACTTTACAAAGGGTGTGC
ACGCCCTCCTTGTTGGACAGGAGAGGTTACAGACAAAGCTGATGGCAGGGTTCAGGTGATGCCACTGTCTGAATTATCACTAATTTTTGAAGTA
GTCATACATATTTCTTGCTGTACCCAGAGCATCTCAGACTTGCACTTGTATCCAGTTCCTACCCCCAGTTCTCTAAATCCATTCAGTCAGGAAG
GTGGACAGCTTCTCCTCGATCCTTTCCTGAATATTTTCATTCCGTTTAAGTTTGACTTCATATGCTGGCTGGCCGAGGCTTATTTGCTTCACCA
CAGTGACTTCTACATCAAGTGTTTCTGCTTCCCACCTGGTTTTCCTCAGACGCAGCCTCTGTTACAGCGCTCCCTGGTTTAAGAGCTTGTCACA
TTTCTCTCTGGATACAACCTGTACAGTGGATCTCAGTCCCATATAACCGAAGGTATGGGTTGGGCCAACATAAATTTGGCAACAGTCAAAAGTT
TCTGAACTTTTAGAATCTTCTGAACTACATGAAATTAATTCAAATTAAACCTCATAAAGACTCTGCTATGTTTCCGAGGAACTCACCAGCTTTG
GATCACAGGAACTCCCTGCAGCCTTGTGTCTGGAACACACATGTGCTAGCCACAAACACATGCCATCACACAACGCCAGGCCAGTATCTGCAGC
CGAAACGGTTCGTACTTTACCTGTGCTTTGATTGTGTGGATGAGTTTTCTGTCCTTATAGAACATTATAGCACCTAATAGCATTCCTCAGCATT
TATGCATATAGTTAGTCTGTCTGTCTTTCTTTCTTTCTTTCTTTCTTTCTTTCTTTCTTTCTTTCTTTCTTCTTCCTTCCTTCCTTCCTTCCTT
CCTTCCTTCCTTCCTTCCCTCCTCCTCCTCCTCCTCCTCCTCCTCCTCCTCCTTCTTCTTCATCTTCTTCTTCTTCTTTTTTAATATTTGAGCT
GCTAATTTGAGTTTCATTTAAAGATCATTAAAGTAATTTTGATCTTGTTTAGGATCAGAATTTCTAACAATTTCTAAAACAGACCCACACACGC
TTCTGGTATTTTGCATCACATATTTATAAGGCAGCATTTTCAGCATTCAGTGTTCTGCCTGCATGCATGCCTTCAGGCCAGAAGAGGGCATCAA
ATCCCATTGCAGAGGGTTGCGAGCCACCATGTGGTTGCTGGGAATTGAACTCAGGACCTCTGGAAGAGCAGTCAGTACTCTTAACCACTGAGCC
ATCTCTCCAGCCCAAGAATTATTCTAAAATAATTTCTAAAATAGTTTCTCTGAGATTTGCTATTAATAAATGGTTTGTAAACCTCTTTGTGTGT
GTGTACACACATACATATATATACATATGTATATATATAATAAAAATATAATAAATACATTACTCAAAAAAAGAGCACCTCCCTCGTGAAACAA
AATACTCCATTGTCAATAGATTTTTTCCCTATTTCCTTTTTTTCCTAATCTAATATATACCCATATTAGTCAATATTAGCTACTTTACCAAACA
TAGTAGTTCAATAAAGGGGAGTTTTAAAAAATCTCTACCTTTTTTTTTTAACTGTCTGCTCTACACAATCACTCGGGCTCCTTCTGTCTCTTTT
CTCAGCCATCTCTAGAAGCCTGCTCAGCAAAGGGGATGGAATTACAATATAGGATCAACAGCTTGTAGTAGGATCGACAGGTTAGCTTAACATA
AAACAAAAGTGCTTTCTACAAGCCTCTGGCTGAGGGCAGGTGAAACATATCTCTGATGAAGAAACGCTGAATGCTTACGTGGCCATGCAGAGAC
CATGATGGTGAAGGCAGCTCTCCCAGAGGGAGGCTTAGTTGTTGACCCTGTGACCTCTCCAGATATGGGAAACTCAACTGCATAGCTGTTACAG
TGAGGAACTGTATTCAAACTCTCCTCTGTTAAATAAAATTAAAAGAAAAGGAAAAGGTGAGAGACCAAGTGCAGCAGAGCAACTAAGTTACAAG
TAGACATTTTGGTGAGAAGAGGTATTAAGATGAGGTCTCACTATATAGCCCAGGCTGGCCTCAAATTCACTGTGGAACCCCATCTGACTTCACC
TTATGGCAGTCCTGCCTTAGCCTCTCAAGCAACAACATCTCTGGCTAGCTGCCATCAAACCGAAGAGGTGGAATTATAACCAAGTTTCAGAATA
AACTCCAGTATAGACCTTCATAGGCTCAGAACTGTAGTGTTAACTCCTGAAAACAAATGAGTGTGTGTGTGTGGGGGGGGGGGTCACAGCTAGG
ACAAGGTGATTATTTACAATACTGACATTTCCAATCCATAGACATGGCATGTCTCTAGAAAGGAGTATTCTGGGCAGAGGGAGAAGAACGAAAG
CAGATAAATAAGAAACCATCTTGTTTATACATCTCAGGGTCTATAGAGCAGTTCAAAATGTATAAAATATAAGATATAGATTGTAAGAAATGAG
GCATGGCCTCCTGGAAGCAGCCAGACTATGGAAGAGTTTGGAAGCCATGTATTTGAACCCAAGGACGGTTTTAAATTGCAAATTTGGTGTGGCT
TGATGTGTATCCCCACAGGTAACTGGGGCAGCAATATATTCGAATATATTTTTAAAGGGGACAGAATTAAAAAACACTTAGAACACTCCAGTAG
TTTAGAAGAAACCTAACAAAGATGTCCCAATGATGATGGCAGTCAAAATAAAGAAGAGGCATAGCTGTTGGCATAACTCAGTGGCAGCCTTAGC
ATGCTTATGGTCCATATGTGCCCTAGGATAGCTATGGGGCAACCCAACACAAAACCATGAACACTTAAAACAGCATGAGCAGATTGTTTTTGCA
AGTTTTTTTTTTTCAATCTACCTATGCTTTTCCCAAATGTGAACTTTATAGGTGACAGTGTATCATAACAATGTCAAAAGACTGTACATTTCTC
CAAGCCTTAAATTTAACCCCTAGCAACAGAAGAAGAAGAAGGATTCATCACAATAGACTATATGGTGAATGGATACTGTAAGGTAGGCTGAGGT
GATAGGAGGGCCAGTCATGGCTCTCAGTCTGCAGAGATATCTCAGCAAGTAAAGTGCTTGCTGAACAAGCATGAGGAGCAGAGTTTGGACCCTC
AAGAGAAGCCAGATGCAAGTCTGTAATTCCAGAGTGCCTTACTTCAGCTAGAGTAAAGGGTACAGAGCCAAAACAAAGAGGCCATCTTCCATCC
CACACTTACACAGGTGGTTCTCAACATGTAGATTGTAACTCCTGAGGGTGGGGGTGGGAGGTCGGGGGTCAAATGCTCCTTTTACAGAGGTCAC
ATATCAGATATACTGTACATCAGATATACTGTACCATGATTCATAACTAGCAAATTACAATTATGAAGTAGTAATGAAAATAATTTTATAGTTG
GGGTCACCACAACCTGAGAAACTGTATTAAGTGTCACAGCATTAGGAAGGTTGAGAACCACTGCTTTAATAAAGTCTTTTAGAAACAAGTGAGT
AGTCAATGCTCTTTAGCAAAGGCAACGCCTACATTCCTTCCTTGGTACAGCAGAGGTATCCTGTTCTAGCACCATCTGCACACTAAAGCTCCAC
CCTGGTACCATTCACTTCCTTTACTACCTAGTCCTGACTTTACCCTGCACGATGAATGGCCTGGGAACCCAACTGCCATGCCAGAGACTGAAGT
GCTCACTGATAGTTGTGTTTGTTTGCTCCTTCTGCATTTTCAGTCTTACTCTGTAGCCCACATTGGCCTGAAACTTGTAACAATCCTGTTCCAG
TCTCCTAAGTGCTAGGATTATAGCTATGAGCCAATAAGCTTGCTTCTCATTTTCCTTTAGCAATTTCTGTTTCCCAATCTCTTTGGAGGCTAAG
GCAGTATATGCTGTCCTGGTGTGCAGTAATAGTAAGGCAGACTGTGCCTGGCGCACATTCATCTTTCACTTGTCCTCCTTTTCCCTCCCTTGGT
AAGGCAGCCTTAAAGGTGTTAGACATTTGTTATAGGCACGTGTGGCAGAAACATGGACAGACATATTTGTCTTTCTATAATCTCAGAATGTACT
GAGTAAAAATAAATCCACAACCTACATCAGTTTTGTTGGCTAGAGTTTCCCAAATCTCCAGCAGACAGTTGGGAATCAGTTCTTTTAACTGTAA
TTATTAGCATTAATTCTCAGACCCACAGTATATACATGTTGTGTGTATATATGCACATATGTAACAGGATAAATGATGTCTAAGAGAGGCTACA
GAATTCTGAGGCTTTCCTGAAGTCAAAGAAAGAAAGCAGATACAAGCAACAGGAAGAGTCTGCATGCATTGGACTAGAAGTCTTACAAACTGCA
GGCATGGACCGGTTCACATCAAATACCAGTCCAAGTGACAGGACATTTCCAGGGTCAGTTTGATGTGAGCCAAGTGTCACCTGCCGGAGTCTCT
TTGCTTATGCTGGAGACTGAGGCAAGGGCAAAAGGAGTGCTTGATATAACAATTATCCTATATGAGGCTTATGATAGGTAGCGGGGACAATTGT
CATACCTGTCATGTTTTGGTCTTGGGGCACTACTTCTTTTCTAGTCCCAGGTCAGGGGATGGATTACATGTAACATGTCAGTTCATGTGTGCTG
TGCTTCTGATTACCATTTGATATATTTAGCTCTTGGTTTATGTTTCACACTGTGCTGGCAGATAATGATTGTTTGCTTGTGCAAGCAAGATATA
GACTCATTCTTTCTTGGCACTCATACTCAAAACAGAGTAACTCTGGTCATGGTTGTTGCTTTGTAAAATGGAATAACTCAAGGCAAGTATGGTG
GTTTGAATGAGAACACTCCCATGGGCTCATATAGTTTAACTTGGTCTAAAGTAGGTGGAACTATTTGGGAAGGATTAGGAGGTGTGTCACTGTG
GGTAGGCTCTGATGTTTCAAAAGCCATTCCCTGTCAGCTTTCTGACTTGTGGTTGTTGTCTCAAGATGTAAGCTCCCAGCTAGTGCTCCAGTGC
TCTGGCTTTCATGCTCCCCACCAAGATGGCCATGAACTAACCCTCTGAAACCATGAGCCCTCAAATTAAATGCGTTCTTTTACAAATTGCCTTG
GTCATTCCTTTGTTTTATGACTGTAATAGAAAAGTTACTGAGGTCACTAGGCATAGCCTAAAAGGTGATATTCCACATCATAGACCTGGTCAGA
GCAGTCCCAGCCCATTCTCTCAGCATTAGTATGCAGTATGCAAGGTGCCTGGATCACCCCTGAAAGGGAGCCAGAGGTTGTTACTGATAGCCCG
CGCACACTATAAGTCCACACCCTGCATCAGCTAATACTCCATTTCTGTTTTCTTTGACACTCTTCTAAAAGTTTATACCTACCACATCCATCAT
TCTCTCCTCAACCTGCTTCAATTTGGCTTTTATAATGATCCTTTTAAAGGCACACTTCTCAAGTCACAAATGTCCTCCATTCTTTATCTATTTC
ATAGACAGGGTCTCCCAAAATAGCCTAGGCTATTCTTGAACTTCAGAATGTCTGGCATGAGCCTGCCTAGTCCTGGGATTACTGGAAAGAGCTA
TGCCACCCAATTTAATGACCTCCATTTTGCCATACTCATTTAGAGTTCATTCAGAAAAGAATCCTCAACTGTTTCACTTCTAGCAGCTGGCCCT
GCCATCAAACATGCCTGTCACCATGTCTTCTGTACAAACACTTGTCTAAATCACCTTTCTAAAATTTCCATCATGGCAACAGTGCCCCACCCTC
ACCCCTAGGTAGCATATACTAACAGTGTACCATACCAGCCAGGGTAAGTCTTCAAATGTACTGCATCAACTGCATGACTAAAGTTTCCAGTAGC
AACCACTGACTCAGCTCAAACATGGCCTCTACAGAATGTCCTTCTAGAGAGCCCAGTTGGTTTCTACTACCCCCACTACCTTATTTTCTTTATA
GAAATGCAGTGACATCAAGACTTATGCATGCTTGTGTTTTGAGACAGACTCCCTCATAGCTCAGGCTGGCTTTATAAACTCATTATATAGTGGA
GGATGACCTAGAACCCCTGCTGGTCCTACCTCCACCTCCCAAATGCTGGGATTACAGACATGCCACCCAGTACTAAAATTACCACGTGTTAGTC
TCTGTAAACCCCAATGACAAGCAGCCTTATCAGGTGTACAGTGACACCTTCCCCTAGCACAAGGTCTAAAAATATCCATGAGAATTTCAACCTC
CTATTTACTTTTTCTTTGAATAAAGACAGTAAGTAGAAACTATGTCATTCAGATATGGTTGAGTTAAAAGCTGAAGTATGCCTGCATTCATGTG
TGTGTGTGTATTGGTGTGTTCAGTGTTTTGTGGCTCATCCTATCTTCAAAGTTATGATTCTGTGGTCTTGACCTCTGGAGCGCTGAACATAGTT
CACCCTGCTAATTGTTTTATTTTATTTTATATCTTTGTGACTTGTTTATGATTGTGTTTAACTAAAAACACAACTGCTATTTATACTCTTTGAG
AAGACAATTTGGAACTAGCAGATTTTAATTATTTCCTTCACTTTGGGGGAGTTGTATAACAGGATGAATTTTTTATGACAGGATAGTATATATT
TTAATTATCCAACCCCCCCCCCCCACACACACACACACACTTTGGCGTTCTTTGAAGCGCATGTAGAGGTAAGAGGACAACTTTTGGAAGTTGA
TTCTGTCCTTCCATGGATCAAACTCAATTTGTCAGGCTTGGTAGCAAGCGCAATTATTCTCTAAGCTATCTTGTTGGCCATGCAAGATGATATT
CTCTATTTCCCCTCCATTACTTGTTTTAGTCAGTAAATGACTGGAGAACGTAAGAGGTGGAGCCAGATAACCCTGAGTTATTTCTGTTGTTGAG
ACAGTCCACCTACCTCAGCCTCCCGCGTGCTGGGAGTGCAGTGCGCTAAACGGGGCTGGATAATCTTAAGCTTAAATCGTGCAATTCCTTTCTA
GAACTCTAAAGTATGGACAAGCTACTCAGCTTCTCCAAACCCCAATCAACCCTAATACTCGTGTATACAATGACATGCTAATACATCATAGACT
AAGAGAACAGGTGCGTATTAGCAACTGAAGGCAAACAAGATTCCGCCTGACCTGTCAAAGCACCAAAGTTCGGTCCAGGCCTCAGGCCGTGGGT
ATCTCGGCCAATCTCTGGACGTCGTGTACATTTATACAGAATGGGGTTAGCGGCCTCGCAGTGCAGCTGAAAAACTTTGGTAGGTAAAAGACGT
AATGTAGATATAGAGGGAAAATAAGGCTGCGGGGGTCCCCGTATGCAAATCTGCGCCTTACTGCCGAACGCGGGCGCTGGCGCTTCCGCACTTC
CCCTCCAGAGCACCCGGACAACTCTCTGGCGCGGGGGCGGAGATTTGCATGTGCGAAATCGGGAAGAGGGGGAACCACCGGTCGAAAACCCCGG
GCGGGCCTCGGGCGGCCAAGGAGCGCGCCCGGTGCACCGCCCGGCCCGCCTCCGGCCGCTCGGCCAGCATGACGTCACGAACACGCCCCTCGGC
TTTAAGCGGCGCTCTCAAGGACTCTGGAGGAGGGGGATTTCTCAGGGACCGACGGCTTCTCACGCGCGGAGTCGTCGCTGCGCGCCCTCACGGA
GCTCCCGGCGCGAGCGTGCAGGCTGTGGTGGCTCTGGAGGAAGTAAGCGCACTGGCCCGCGCGGGGTCGCGCGCCCGCCGCGAGGCTCCGCCCC
CCGCCCCGGGTAGGCGGTGGCGCGAGTGACGTAAGCAGCGGGAAATCCCCCCCCCTCCCCCCACGTTCCGCCGGGCGGTCCGCCCCGCCCCCTG
CCCCTGCCGCGGCATCCTGGCAAGGCGACGCCAGGACACTAGTGACCGGAGCGCGAAGATTCGGGGGTGGGAAGGTGTGAGCCGCAAACCCAGA
GGAGCCGGGAAAGAAGAGGAGGCCCGGGGGTGGTCCGAGGACAGGCGTCCCGGCTGCGGAGGCTGCTCAGCCTCCTCACTGGTTGGCTGACTGA
CGGGCTAGCTGGCTGACTGCCTGCCTGACCGACTGCCTGGCTGACTGACTCACTGACTGACTGACTGGCTGCCTTGCAGCCTCGGGCCGGACGC
TGGAAGCTGCAGCGAGCGGCGCGCGGAGCCGAGCCATGGCCTCGAGTGAGTACCGGGGCGAGCACTCGCGGGTCACGGGGAGGAGCTGTCGGGG
GCTCCGTTTGGGGTTAGGACGGTCTGCCTTTCAAGTCTTCTCATCTGGCAAATTGTCAGCCGTTTGAAAAGTTGCCCGCTCCGGGGTTTGCGTG
TGAAATCAAAGCGGCGCACTGTAATCTGATCTTCTCCTAATAGCCAGAACCTTCGAAATTCCCAACGGTGTGCTTTTAGGATCACAGGGGGTAA
TCTCAGTAAAGGAGAGCAGAGAAGCCTCGCGTTTAAAGACTCAGTGGTATGGAGACCCAGTTTTACATTTTTGAACAAAAAAGAGGAACCTCCG
GACCTCAGACGAGCTTTCTTTTTTTAAGTCCAATCGTGCTAGCGGCTGACACTGGTACAGAATGTGGCGTGGCTGTGAAAGTCCTCAGTGGCGT
TTTAATTCGTTCCAAGCGAAAACACTACCCCCGCCCCCACGAGCTAAAGATGTTGCTTGTCTAATTTACCGTGTAGAGACCTCGATCCCTTCAA
CCCCCAACCCCAAACCTGCACGCGCGTTTTGTTAGCAGCAAGAGGACGAGATTTGATTATACTTTAAAGTAACTGGTAGGGTTGAAAATGTCAT
CGTTTAATGTCAGTGTTTTATCTGCCACTAGTAAGTTTTGGATCATCTGCTTTGAGGGGGACATTTATAAGAAACAGTTTACAGGACAGGGGAG
TTTAGTATGACAGTCGTACTATATGTAAAGCAAGCACTTGAAGCAGCTGAGATGGTGGTGGGAGCTAAAAATGACACCTTCTGTCTGTAGCAGG
ACGCTGACCCTTATCTTCCCAGTGACCCTCAGTAGAAGGCAGCCGACGTCTGGCAGAGAAGAGGAAGTAGAAAGCTTAGTGAACTCTTTAAGAG
TTGAGCAGAACAATATCATAACTCCTGCTGTAAAGGAAACTGGGGTGTTTCACATGACACTTGATTTCAGTTTGTCTGTGGTTTTCATGAGAAA
ACAAGTGTAGTTTAAAATATTTCAACTTTCTCTTTACCCATTTAAATGAAGCCAGGCTTTCAGATGCTCTAGTAAATTTGTAAATTTAACTCAT
GTTCTGATTAGAAGCACAAAAAGGCTTTAAGTATTTAATTCATTGGTAATCAGAAAGAGATTTTTGTATGACATTTGTAAAGAGTGGCAAGCTT
TGTTAAAACAGTTCTGTCCTTGTCTACCTAATAGAATCTTATTATATAGTGTATGAATGCAATACTTGAATTTTTTTCAACAGAAAAGTCATTT
ATTAAAGAACTGTAAGACAGATATGATTTTAAAATGTAATACAATTGCAATTTGCCTAAGGACATATATTTTGTGCTGTTATAGAAATGATTCC
AAAAGTTAAAGTGGGGAACTTTTTAAAAGAAATGGCAAGTTTTAAGGATTTAGAAGTTGGGTTGGGCTTGCCCAGCTTCTGTGAAGCCCTGGGT
TCAGTTCTCAGTACTGTCAGAGAAAAAAAAAAAATCCCAACAAACATAGAAAACAAATCTGCAAGTTGTAGTATCATTATCTTCTTGCTTTTTG
ATCTTTTATCAAGGGTTGCACTAAATGAGACTACTTAAAATACACCATGTCATTTAAATTGTAATTCTGCTATTTGAAGTTTCTAGGCAAAAAT
ATAGTTTAAAGGAGTCTGGGTACCTTATATCAGTTATAAAGACCAAATAGAGAAAAATTATGAAATAAATTTAAAATTCCCATTCCCTTTGAAA
TACTAATTTAAAAGCAACAAAACTGTTTAACTTATGATTATGTAATTTAGATCCTTTTTTTTGCAATATAGTACTGAAACCTAGGAAAGTATTC
ATATTGATTACCAAGCATTGAGATCATTTGTTCATTCCTCTCCGATGTCATATGTAAATAGGTATGTGTAATTGAATAGGTGGTGCTTATAAAT
GTAAAGTGTCGTAGATAATAATCTCCTGGGCAGAATTATATAAATGTTGTGAAAAGACAGGATTTTATATGATGATACTAATTATTGTTCAGGT
AAATTAGCAAGTTAAGACTTCCTAGCTAGCCATTAAGCTGTTTTTCATTGCCTTTTAAACAATACTGTGCTTTGTTCTAGCTTTTTGATAAGAT
TAATTATAGCAGGCAACATGGGAAAAAAATGTTCCTGATCAAAGATCTGCATATTTAAGCAGTCATTATTCAAACAATTGTATTCTATTTAAGT
TAGCTGTCTTGTGTCCATATCTAAAATTTTCACTATTTCNNNNNNNNNNNNNNNNNNNNNTGGAGGGAGGTGCACAGTTGCCTTGTAAGATACA
TACTATTTTACTAATGATTAAGATTGTCTTTGGTGAAATGTACTTTAAAATTTAGAAAGTTTGTATTTAGTCACATTTTTTTTCTTTAACATGA
TTCTGCTGTGTCACATAACAGGAAGTATAGGAAGGAATTATTTTTGTGGACCTTACTACTCACCAACTTTTTGTATGAATTTCAAGATAACCTT
TTCCCCCCCCCCAATAAAAGTGAACATAGCTGTGTGACAACTTACACTTTAATAATAGAGCTGGAAGTTAGGAGTGGCCAGCTAAAACAAATGA
CTTCCTTTTTTAAGAAGGAAGACTGTTCACAAATACAGATGTTTCAGAAACGCAGCACATGAAAAAGAGATAAAGGTGTTGTAATAGTAGGTGA
TGTAACAACAAGAATGAATGGAGAAAATGGTGGATACAGGGTATATGTGGTCTCTTTCTCAATTTTTTTGTTTCAATTTTCTCCCATATGGGTT
CTCTGCATGATTGTGTTTATGGGCACATGAGCTGAATATTGCAGAGGAAGATATATTTTCAAAAAAAGTGTTCCTTTGAAATTTGTACTTAATT
GGATCTCTTGTATTTTTAAGTCTGAAATCTTTTTTTTTTTAAAGATTTATTTATTTATTATATGTAAGTACACTGTAGCTGTCTTCAGACACTC
CAGAAGAGGGAGTCAGATCTCATTACGGATGATTGTGAGCCACCATGTGGTTGCTGGGATTTGAACTTGGGACCTTTGGAAGAGCAGTCGGGTG
CTCTTACCCACTGAGCCATCTCACCAGCCCATCTGAAATCTTAATAGAGATGATTTGGCCCTGCCATGAATTTTTTATTTGAGGAAAGACTTTT
AAACATTACTTTTGTATTCTAGTAGGTAAAAACCTCTCAGGACTGGTTTTTAGTTTGTTGTTTGTTTTGTTTTTCCCGTATCCACAGCAGTGAT
ATCTTACTGCAAACCGTTTACACGGCTTTGGTTGGCCTTGTGATGAATACTCAGCAGAAAGTTCTGGATGGAGATGATATTTGGAAGTGTATGA
TTATTAGCTGCTTGCTCCTTTGCGTTCCAGGCAAAAGCTGTTTCCCTAGGATTTCATGAGAAAGAAGCACACTAGTCAGCTCTTCTCAACCATG
CTTACTATGACTGCCATTTCTGTGCTGCCTTGTAAATAACAAGGCTCAGATATGTTTTATATAAAGGGAAATCAAAGGCCATCTTTTGAATGTT
TTCTTTGTTTCAGTTGAATGAGGTTGGTTGGTTGGTTGGTTGGTTGGTTGGGTGACTGGGAGAGTGACAACTTGCCAGACATTTTTTGCCTGTC
TTATCCATAATTATGTCTATGCTTGGATCCGAGCATTGGGTTACTTCCCTGAACACTTTGCATTGTTGTTTTTACTTCTTCATGTCTTAGGCAG
TTGGTCTTCAGAGTATTCAGATTAATTTGGGATGTTTCTGCAGAAATCTGTCTGCACTTTATTCTTGAAGTGTTTCATTTCTCCTCAGATCTCT
GTGAGGTTAGATAGTGGTTTGGCTTCTGATATACAGCCCCAGAACAAACTCTGTTCTAGGGACTTTGAATCTAAATATTCACCTTATATTCTTA
AGATAGTTGTATTTGTTTTAGGTTATAGGTTATGGCCATATCTGTTATTGAGGTTTTATGCTTAACAACTTTTTTCTTGTTACATAAATTTAAA
AGTTGATTTTATTTTGAAACTTGGTTGTAATTACACCTCTGCAGTTTAAAGACACCTATGACTTTTATTCTTTAAAAAAATAATCTCATGGAAG
AGTTTCACGCTATCATTCTTTCCCTAGCTCTTCTGTGGCTATGGACCCAGAAGACATACATCCATTTGGTAGTCAGATTTGTTAGTTCCTTGGA
TTATGACTGCCTCACTGCTATATTATTAAAAATTGTTACTGTACTGTGTATATATGTGTTTATGACAGGCATAGTGGTCCATTCCTTCAATCCC
AGCACTTGGAGGCAGAGGCAGGCAGAGCTCTGAATTCAAGGCCATTCTGCTTTATAAAGCAAGTTACAGGCCATCCAGACCATACAATGAGGCT
GTCTCAAAAAAAAAAAACAATAATAATAATCCTTTTCCTCAAAATTTTATATTATTTTGAAGTTTTAGCATCTCTAAAATGAACAGCAGTTCTC
TTTATGAAGAGTAAAGTTAAGAGAATCTGTGAGAAATACCTATGTGTGTGTTTTAATTGGTATTATAATTAATTATATGTAACATATTCTCATT
GTGTACTTAATTATGGCTTCTCATTCTAGCAATAGTATTTTTGTTGATATACCTATTTTATATATAATTGTGGCAATTGATCTATTTTTAATGT
TTCTAATGTGAAAGAAATTTCTAATTCCTTTTTAATCTTAAAATCTAATTTTTAATTTTTTTAATCTTAAAACTCATACTTGAGATTTTAAATA
GGCTCAGTGTTTTCAAATAGCATTCTCTTGCACCTAAATGCAAATGAAATCATAACTATAGTGCAAGCATTTTAGTTATAATTCAAAACTTATT
CTAGTAGATATTAGCAATTCAAAATTTATAAAAATATATTTAAAAAATATGGAGACCAATCCAGACTTTACACTTGTCCTGGTTTAAAGAAAAT
ACCTTAATTAAATTGTTTAGAAACAACTTTGAAAACATTTCTGTGCTTTTAAAACTCATAGTGACTTTCTGTAACCTTAGGTAAGAGTATTTGT
TTTCCCTAAGGTTCCTTGGTTTATAATTTTTCCTTCGTACTCTCCATAGAGGACTACAGTTTTTAATTTTTCATCTTCAAGAGGAATGGCATTG
TAAAACAGTACCAAGGAAGAACATGATTGGATCATTCTACTGACACATGATACCAATGATTTCCTAGCTAGGAGGGCTACAGTTAAATCAGAAT
ATGAACACATATGAGGAAGCATGAAGGTGTAGTTGTTCTGGTACCTACAAAGTACTGGGTTTTTTTTTCCAGTTATTGAGGAATATAATAAGGA
GGGACTGATAATCATTAATATAGTCCTTTAATTTTTGAATCACTGACACAAAAGAGCTTTGGAATTTTAATTTTCACACAGATATTAATTTAGG
ACATTAATAAGTAATACAATTTCTGCCATTCAGTAACTTGAAGAAGTAAAACATGTAGTTTCATTTCAATAACAAAGCTGGGTATGGTGGTAGA
AATCTGTAGTCGTGGTATTTGGAAGATCGGAAGTTCAAGGAAAGCCTCAAGCTACACATTGAGTGAGGCCTGCGTAGATTACTTGAGAGCCTGT
CTCAAAAAAGGATGTAGCTCAATTGATAGAGTAGCTGCCCAGCCTTGAGGAGCCCTGATTGGACCCAGCATTGTACAAAGTCTGTGGTAGTGGA
GCCTATAATTTCAACACTAGGAAAGTGGAGGCCAGAGGCTTAGAAGTTCAAGATCAGCCTAGGCTACAAAACAAGTTAGAGGTTAGCCCTAGAT
AGATAAGACACTGTCTCAAAACATAAAGAACACAATAGTTAATTTTCCCTAAACTAATGGTTTTTTTGGGGGGGGGGTTGGTTTTGTTTTGTTT
TGTTTTGCTTTGTTTGGAATAATTGCTCTTAAAAATTACCTTGATTGTTTGTTTACAGGACGGAAAAGTTATTCCCAATAGGGAATGTAGACTA
TAAATTCTGAAATTTGTTTTTGAGTGAGACTTGTTATCAATGAAGAGTTCCTTTTTATTGTTGGTATATTTTTGATTTTTTGTTTTTTGAGACA
GGGTTTCTCTGTGTATCCCTGGCTGTCCTGGAACTCTCTGTAGTCCAGGCTGGCCTCAAAAATCAGAGATCCACCTGCCTCTGCCTTCCCAGTG
CCAGGACTAAAGTTGTGCCCAACTATTTCCTGGCTCAATGGAGAATTTCTACAAGAAGAAAAATAAATATAAATGCATGAAATTACATGACAAT
ATTTAGGTAATTCTTATTACTTTAAGAAATTTTAAAAATACTTAGTCTTAGGTCCTTTGTATTTCATAGTTTTATAAATACACTCACTGGTTTA
AAGAAATGTGTAACTTGCTTTGAATAACCCTGCAATGTTTTTTATATTTCTCCTTTAAAAAAAATCCAGCTATCATTAATGATATCATTAATTA
TCAATTTCTCACCAGAACACCTTTTAAGTAGTGGGCTGGGATTTATTTGACCATTTAGTTCAACCATATAAGTTCTTTAAGATGCCACTGAAAA
AAATCATGTTGAATTTAACACATTTGAAGAGAAAGGTACATTCTCTTAAGTAGTCCTACAAGAATGCATAGGCATATAAGATATTTAAAATTAC
GATGTAATTTTTACATATACAGTGATACACACATTGTAAAATATTCATTTGCCTTATATTTCCAGTAATTACTATGGACTTTGTTGGATTAAAG
TAGTTTTCTCTTTTGTGTGTGTGTGTGTGTGTGTGTGTGTGTGTGTGTGTGTGTTTATTGTTTATTTGTGTTCTTTTGTTTTGTTTCTGGTGGT
ACTGAGTTTGTACCCAGATCCTTCAGCCTGCTTTCTGTCATTTGAATAAGCAAATGAGTGATAGATTCAAATGCAAGAGGTCTAAATCAGAATG
AATAAAATTAAAGGTAGCAGCAATAATTTATTGAAAAACCACATGGCCACAATTGATTCTCCCCTTTGCCCCCACCCCCTGTGTGTCTCTCTCA
CAGGGACCTAGTACTCACTCATTAGGCTAGTCTGGCAAGCCAGTGACTGCCAAGGTTCCTCCTATCTCCTCTCCAGCACTGGCTTACAGGGATA
TGCTACCATGCCTGGCTGTTTTTATGGATGCTGGGGCTTGAACTCAGGATCTTGTACTTGAATGTCACGCATTTTTCTGACTGGGCTGTTTTCC
CAGACTCTTACCACTAGTGGTTTATTTCTCTTTGATGTTTAGATACCAAAATATGTCAGTTTTGTTTTTTGAAATAATCTTTTTTCTTTATAAC
AGCTATATTTCAGACATATCCACATTTTTGCTCATATGACAATACTCTCCACTTACCTATAGTCTGATTTTTTTCAGTTCATTTATTTGTAATC
AACTGTAGTCCAAAATGTTAAAAGAAAAATTTCATAAAGTTTTAAATAACTTGTTTTAGTCATTTGTTGTAATTCTATTATATTGTTGTTAACC
TCTTATATGTGTGTATGTATGTATGTGTGTGTATGTATGTATGTGTGTGTGTGTTTATAGGATAAAATGTATGTGCATGTATGTAAGTCGTCTT
AAACTGATTCTTTTCTTTTCTTTTTTTTTTTTTAGTCTAACCACTACCATCAGAATTCTTTCTCTAGAAAGCTAATCTGAGCCTTTGATTTCCT
GAATAAAAGCATTGATTGCTTTTCACATATACAACAAAATCCATATTACTGGATGTGCTTGGACTTTTGGTTCTCTTTCTAAGAGTGGTAGTGC
ACTCTACTTCCATTGGTGGTTGATGTCATGGACAATTACTTCCCTTTTTCTAAAATGTCTTTCCACTCTTGTTTTGTTACATTTTCCCTCTCTT
AAACTGTTAGATTCCTCTTTTGTTCTTATTATAACTTTCTGCAGAATTTTGTTAGTATTTCCCCCTTCTGTACACTAACATCATTGCGATGACT
AGTATAGTCATTGCTTGCTGTTGGGTATAGGGTCCTTCCGAGTAGCAGCTGTGATAGTGAGGGTGTCACTTTCTTCAAGAGAGCAACATCTGAA
GTAATAGTAGTACCATTTACTGATTACCACTTGTATGTGTGATACTTGGAAGAATTTAACACATATTTTACAGAGCATATTAATAATAAGATAT
TATTAAACACAGGGTCTTGCTAGACTCTTGCTGGATCTTATTTTGTTTTGATAGTAGAACTCAGGAGGCAAAAAAATCACTGCAAGTTCATGGT
CAATTGGGTCTACATATACATAGTAAATTCTACAAGGAAAAGAAAAAGTGCCCAGTTTGGAATAGACCATGTCCCATGCTATACTAAAAACTCA
TTGTTTCACCATTTCTGAAGCTTGTTTACATTCTACAGGATAAGCCTTAGTGAAATGGGTTTATGGTTTGGGGAATTTATACTCCTGAAGTAAA
ATCACATGGTTACCTGCCTTAACATGAAAACACAGTTATTTTATGGACCTTCTATTTCCATGACTTGGCTAAAGGGTGAAGATGCATATATTTT
TAAAGTTTTATTGACATATACTTAAAACTCATTATAATATAATAATTATATATAAACTTTTTTTTTAGTTTTTTTTTTTTTTTGATATGAGATC
TCATTATGTAGCCCTGCTGGCCTGGAACTTGCTATATAGTTAAGACTGGTCTTGAACTCACAGAGATCAGCCTACCTCTATTAAGGCCTGGCCC
TCCATACTTGGAAATACCATCCTTCAAACTTTCTCATCTACCAAGTTTAGACAAATAGTGATCACCTTTTTTTTCATTATAGATTAGCTTACAT
TTCTGGAGTTTCATGTAAATGGATCATATAGTATGTGCTTCTTTTACTCAACACACTTACTTTGGTAGTTATATTTATAGTGTTTTATCCTAAG
GGTACGTATGTTGAATTACATTTGTTATGGAAGGTAAATGACTTTGTTATCTCCACATTATGTGTTACATTTTGGAGAAGATTTAGATGGGACC
GATTTGAAAGTTCAGTTGAGCTTAAAGTCAGTATACTGTTTTAGTATATTGTGTACAGATCCTTTGATAATCCAGACTCGCCTATGCAAGGACG
TAAGTCCACTTTAATTTTTAATGAGTGTTCTTTCATGCAGTGATTGTCTAATTTAAAATAAAGCAGTTAAGTACATTTTACTCAGTTTTGGAAT
ATAATGTACTTCTGAATACTATTTTCTTCACCTTTCTTGGGAAATGTTGGAAGAAAAACTTTATAATTGCCCTCATGTGGACAGGTAACTCTAA
GAGTTGTTATCTCACAACTTAATACACTCATTAGGTATAGATATTAAGCAATAAAGAACTCACCAGTTATGTGTTAATACTACATAGGAATTTA
GTTTGTATAAGGAAATGTTATCTTCATCATTTAATCTTTGAAGATTACATTTTACTTTGTTATTTAATGTTGGGAAGGAAAAAAGATCTTTGTT
TTTTTTAAAAAAAAATATATCCATTAATGTCTATTATATTACTATTCTAATCTCATTGCACTGTTCTTTTTAAAATTTTCAGGTGGATATAATC
CATACGTAGAAATAATTGAACAGCCAAGGCAGAGGGGAATGCGGTTTAGATACAAGTGTGAAGGGCGATCAGCAGGTAGCATCCCAGGGGAGCG
CAGCACAGACAACAACCGGACATACCCGTCTGTCCAGGTAATGGCCTTTCTTCAGTATCTCACTTTTAAGGGTTCAGTTTGTTTTACTGTTTTC
TAGACAGGGTTTCTCTGTGTGGTCCTGGCTGTCCTGGAACTCATTCTGTATAGACAAGACTGGTCAGAGATCTCCCTGCCTCTGCCTCCCAAGT
TAAGGGTGTGCATTACCACCACCTGGCAGAAGACTCAGTTTAACAGTCTTTTGCAACCCCCTTTCTTCTTTGCATTTGCTCTCCAATAGCTAGA
TGTGTAGGGTTATCTCTTAAAGGAATCTTTCCTGCTTTTAAAGTTCATGTAGTAATATAACTGCAGTGATTCACAGTGCTTACTGTACACTGAC
AGCTTTGGATCGGATCCCTCTTGTAACACAAAGCATTTAATGAGGAAAAAGCATTCTGTACGTATACTGGTCAAGGGAATTATTGTATAAGTTA
AAGATGGATTTGGAACATTTTTTGACCTTTTAGTATAGAGGGTATTAATAACTTTCCCCTTCTTTTTTTCATACCTTTATGCTTACCTCCCAAA
CTCTGAGTTATTAACATCATTTAAAGTATCGTTCCTTTTCTGTGTGTGTAAAGTGCTCTTTTGTGTGTGTGAATGTGGACACTCGGGTACCTTA
GTGTCCATGTGGGAGTCAGGACAGCATTCGGCAGGTTGGCGTCTTATTCTTGTGTTTTGAGACAGGGTGTCTTTGCTGTTTTTCCACTCTATCT
ATACACCTGGTCCACTAGTTTCCAGGGTTCTTCTCTTTCTGACGCTCATCTCCTGATAGGAGTGCTGCTACTACAGGCTCAAGCTACTGGTCTC
TTTATCTTTATTGACAGTACTAGGGATTGAACCTAGAATATTAGGAATGATAAACAAGTGTTCTACCACTGTTATATATCCCTAGCCTGTGAAA
ACAGTGTTTTTGGTTGAAAGTGAATTTAGCAAATATACCATTTTTATAATGTATGTTTATATTTGATATTTATAAATTACATTTCCTTTTAATT
TTTATAATCTATTTATTCATTTATTCATGTTTAGTGTTTTCAAACAAAATTGTTAGCCCCAAACTTGTAGTGATCTTGCCTCTTGCCTCTGTTT
CTTCATACTGGAATTACAGACATGTGCAATCATGCCTAGCTAATTTTTATCGTTTAAGTGCCAATTAAAATCCTTAATAGCGAGTTGGTCTGGT
AGTACATGACAAGCATTGGGAAGCATAAACAGGTAGATATTGAGTTGTGGACTACTTACCTATATAAGAAGTTCCAGGCCAGACTGGGCTATTT
AAACAAGACCTTGTCTCTACAAACAAATGAAAACAAAGTTAATAGAAGTCAGGGGCAGTGTCACCCATCAAAATCTCATCTACTTGGGAGGCTG
TGGTGGGTAGATGCCTTGGGCCTAGAAACTTGTATGGATAAAAGGAAGACTATCTCAAAATGGAGTTTGAAATGTCCTTAATTTTTCACCAATA
GATCTGAAATTACTGTTTTGTAACTAGCTTATTATTTACAGTTTATTTTATTGAGCCTAAGAAGAATTACAAAATTGCCAAGTTATATATAGAT
TCCCAAATTTCTGTTTTTATATTTTTAACTGATACAGATGCTTAAATTGTACATTTTAATGGGATGTCATGTAATATATAATACATGTATACAT
CATGTAATGCTTCAATCAAGTTAAACATACTTAGCTCCTCAAACATTTATCATTCCTTGTGGAAACATTCAAAGTCCTCCTAGTTTTTTGAAAT
ATATAGTGTATTTGTACAATAATATGCCTAAAGGCTTTTGTCCTATCTACTGTACGAACTTAATTTTAAGAAAATATTTTATATATGCACACAC
ATACATATATATATATGTATATGAATGTGTGAGTATGTGTGTACATGTGTGTGCACGCATAGGCCCATGGAGGCCAGAAGAGAGTATCAGATCC
CTTGAAGCTGGAGTTACAGGCAATTGTGAACTGTGTACCATGAATTCTGCGGTCTGAACTCTGGTCCTCTGGAAGATCAGCAAGTGCTTTTAGC
TGCTGAGCTATCACTCCAGGCCCATAACTTCATGTTTTTAAATCACACTATTTGCAATAATATTGCTTTGAATTATCTAAATTCTTTCTAAGTG
TTCAAAGACATACAATGAAAAGTTAATTTAAAATTCTTTTTTTCCAACATTGTGAGTCATTTTTGGCTTCTCTTAAAAAAATTGTGTTCCTAAA
GCATTCCAGAGCAGTGTGTCTTAACTCCTTACTCCCCTACTCCAGTTCATCCTGGTAGACCTGGAAGTAACACATGCAGCTCAGAGCACAGTTT
GCTGCTGTCTACAGGGTGCAGCCAGACACACAACCAGTTCTGGAGATGGTTCTTCAGATAAATTTTGAGGAGATGTTTCAGATACTTTTGAAAG
AAGGTGCCTCTGAAGAGGCAGGTCTTGCTCTCACTCCTTTGATGGTCACACCCTCCACCAAGATCCCAGCTTTCCCTTCACCTGTGGCGTGGGT
ACAGTCAGGGATGAATTTCAAAACCTGCTTCTATTTTTACTTCCTTTACCACAAAGTTTTTTGTTGGCTATGTGGTCTTAGAGGACTCAGGCTT
TTTTTTTTTTTTTTTTTTTGACACTTCTCTCTCTCTCTCTCTGTCTCTCTGTCTCTCTCTCTGTCTCTCTCTCTCTCTCTCTCTGTGTGTGTGT
GTGTGTGTGTGTGTGTGTGTGTGTGTGTGTGTGTGTTCTCTCCTTATGCTACTGTGAGATACAGAGATTGAAGTTGTTAGTGGCAAATGCCTTT
ACCTGCCGCACCATCTTACCAGCCCTTAGTTCTTCTTAAATCATCTTACTTTATTCATCAAAATTATATGTACAGTTTGTATTTTTTCTGTCCC
AACAAGTCTTTGCTGTAGTAATATATAATCAGGGGTTTCTACTCTGCCTTGGATCATTTAGTTCCCAAATTAAAAACTTAAAACCTTTATATTT
ATGTTAAGCCTTTAAAGCACTAGAGCTGGGCAGATATCTACCCTCTAAGTTATTAGTATCTACTTTCTGAGTTACAACTTGACATGCTATGCCT
GGGCCACTCTTACTGCAATTGGCCAGCCCTTATGGCCATGCTTTCATGATTCACCTACCCCATGGTATCTTTTCCTCTCTCCACCTTCTCCTTC
TTCCCTCATGGTCTCCTGCCTCAGTCCCCACGCCCAGGAACCAAAACTTTACCCATCTCTCTTCTGCCCAGCTCTAGGCTATCTGCATCTTTAT
TGACCAATCAGGGATAAGTTGGGGGTCAAGGTTATATTTGGTGTACATGAGGATCTTCCATCTCCTGAAGCAACCAGGGCCAGTTTTTAGCATT
ACAATACATAGCAACAGACCAAACCTCAACAGTTTTAAGTTAAGGAGCAAGTTTTGTACAAAAAAGGCTGGTAAATATAAGAACTATTTATTTT
GGATTTTTGAGGCAGGGTCTCTTTGTGTTGCCCTGGCTGTCCTGGAACTCACTCTGTAGACCAGGCTGGCCTTGAACTCACAGAGATCTGTCTG
CCTCTGCCTCCCAAACTCTGGGATTAAAAGTGCACCATAATGCCTGGCCTGCTTACCTGCTTTAAAAGGAAAATAAACAGATGATAAAGCTAGA
TAAAATAAGGGCAGAAAGAAACATTTTAGGAATTGAAAGATAAGTTTTCCCAGCTGATATATTCTCTGAAAACTTTAATCTCAAGTAAACTGCT
GACTATTTGAATTTTATTGCATGCTGACTACATTATATAAATTGAACTTAAATTAATGGATCAATTCTTTTTATTTGTTTGCTTTTTAAAAGGG
GCATTATTGTAGGTAGCCCTGGCTGGCCTTGAACTTGTGCTGATCCTGCTCAGCCTTCCAAATGCTGGGGCTATAGGCATATGCTACAATACCT
ACCTATTGTTAACAAGATATACATACAGACTGTATATTTAATTTCCCTTTTCAGATTATGAACTACTATGGAAAAGGAAAAATAAGAATTACAT
TAGTAACAAAGAATGATCCATATAAGCCTCATCCTCATGATTTAGTTGGAAAAGATTGCAGAGATGGATACTATGAAGCAGAATTTGGACCAGA
ACGCAGACCTTTGTTGTAAGTATACAGTGACAGAAACCTTCAGCAATAGGACAAAGATATAATTTTAGTTTATAGCAAAATAATTCTGTATTTA
ACTTACTGTAACATGTACATTACATTTTAATTACACTTTGTTACCTGTGGGGAGGACACACGTGCTTGGGGTGCATGTGGAGGTCAGAGTTGGC
TCAGCTGGTGGAGTCGGTTCTCTTCTCCACCACGTGGACCCTGAGGATTAAACTCAGGTTGCCAGACACCTTTACCCACTGAGCCATCTCACCA
ACCTGATATTAAAATTTTTGTTCTTACCACTTGCATTCATCCTTTATTAATAGGTACGCACATGATAGTTAGTAGAAATTCTTCCTAATGGCAC
CTAGACTGGGCTCAAACTTACTATGTAGAATGTCTTTGTACTTACCTTCCTGCCTCCAACTCCCAAGTCCTGACATTAGAGGTAACACCACCCT
GTCTGGCTCAGTAGGAGTATTTTTTATGTTATTATTTTGTTTTCCTTTGTTGTTCTTTTGTTTTTTTCAAAACAATTTCTCTGTGTAGCCCTCA
CTGTCTTGGAACTTGCTCTGTACACCAGGCTGGCCTCAAACTCAACCTGCCTCTGCCTCCCCAGTGCTGGGACTAAAGGCGTGCCCTACCTCTG
CCTGGCTTCCCCCCCCCCCCTTAAGACAGAGTTTCCTGTAGGCTAGGGTTGCCTTCCAGAGGATTCAGATACTGAGAATTAGAGGTGTGAGATC
ATAGCCTTAAGACCCTGCCTCCAGTTTATGAGGATTGAACGTAAGACTTTATACATGTTAGGCTAGAAATCTGCCAACATATCCACCACTTCTT
ATTTTTTAACTTTTGACTGTTTTGAGACAGAATCTCACTGTGTAGCTCAGCTTAGCTTCCAACGTGTAGTGATCCTCCTTCCTCAGCCTCCAGA
GCACCGGGGTTATAAGTAAGCATGCATTACCATGTCTGGATAGGTATACCTCTTTATATTGTTGAAAGTAAGAAAACATATGTTGTGATTAATC
TCTTCGTTCTGTATGTTCTACAAAATAATTGTGACTATCCTGTAATACACAGACATAATCTCCAAAAAGTTCTTCTTATTAATAGTAATGACAC
AAAACACAAAAAGCCCTAGAAGTACACACTTGACAAGAAATCTTAACGCCTGCATGTCTTGATCTTCCAGCAGTGTTTCAAACTTTCAGTCAGT
CATTTGTGTCTTGAATACTCTTTCTTGATTTCTGTGAAGACAAACTTGTTGTAACCTGTAGTTTTCTGGCTGTTGTTCGATGGCCCCTGGTTTT
TATCTTTTGTCCCACTCTAGTGATATATATATATATATTAGTTGTTGGGGTTTTTGTTTGTTTGTTCGTTTTTGAAACAGGGTTTCCTCTGTAT
AGCCCTAGATGTCCTGGAACTTACTCTTTAGACCAAGTTGGTCTTGAACTCAGAGATCTACCTGCCTTTGCCTCCCAAGTACTAGGTCTAAAGG
TGTATGCCTCCACTGTCCAGCTTAGTGCCTCTGATCTTATACTGCAGTCAGGAGAGTGTTCCTTCAGTCTCTGTTCACAGTGTTCTTTCAGAGA
GATGTGATTTACTTAGTTCACAAGACTTGAGCTAGCAGGGTTGTTGTTTGTTTGTTTGTTTTTAAATTCTAGTAGTATCATAGGCCTTGTAAGC
CCTTTGGACTCTTATGTCCTACTCAGTGGCAGGAAAACATCCACAAAAGATATGTGAGTGAATGAACATGCATGTGTTCCAATACAAACTTTAT
TTGCAAAATGAGCAGTATGCTGAATTTGGCCCATGATTTAGAGTTTGCCAACCGTCCATTCCAGACACTAAGTTCCATGAAGAGAGGAAAATGT
ATCTCCATTCCCTTTGGGAGTTTTAGTGCACAATTGGAACTCCAGAACGTGTTGAATGACTATGAAAAAGTACATTGAGTGAATGATCAGACGC
CCCATGATTCTTAGCTTCCTTAAGTCAGTGCTTTCGCCTCTTTCCCTTCTTTCCTTTCTTTAGTACTGGGCTGATTCCAGGGTATGATGTGCTA
GACAGGTACCACACTACTGAGCTAACCCTCTGTGCATACCCGATGAGGTTATTCAGATGAAAGAAAACCAAGTCGTGTATGTTAGGGTGAAGAA
TTATTTTGTTGCAGGATTTCAAGAAAGGATTAAGAATTGACAATTTATTTTTTGTTATTGGAGGAGGAGGAGGAAGGAAGTGGCAAAGTTATTT
TAGGTGCTAATGAAGTCACAGGTGTGAGTGCCAAGCATGAAGCATGAACATACAGTAATAGGAACATGGAGTAAGGCAAACCAGGCAGCCTCTG
GCTGCAAGTGCCAAGTGAAGTTTGAAGAAACAGTGATGTTCTTATCATTTGAGTTTTGCTTCCTTATTCACACCCACCCATCTCAGATGATGTC
ATGGTTTTTCCATGTGGGAACAAGTATGACTGGATGGTGGTAGGGGAGTTCCCCCATCATCATTTGTATTACACATATGCAAATGAGTAATATT
TGTAGCTATTGAGAAGTTTTCCATAAATGACCCTTTGAAACTGTTGCTAAATATTGTTAGTATTAACCCTCTCTAATAAAAATACTTCATTTCC
TATATTGTAATCATATAACATGGCATAAAGTGTATTCTGTTGTCAATGAAAAAAATTGAGGCCAGGCTAAAGTACACAGCAAGACCTTGTACAG
CTGTGTTTTCATATATTTCATGGTACATTTTTTAGTGATGAGCTAGGCAGGCATCATAACTGAGCAGAGGGAATCAAATATAAGAAGGCTAGCC
CGGTTCCTCATCAGAGTGGATAACTGTGTGTAAGTGGGGTTACAGGTGCTCAGCTCTAACAGATTGTCTCTGTGCCCAAGGAGTGCTAGCAAAC
CTCTGAAATGTTCATGAAACCAGAAGAGCTGATGCTCAGCTCTGATCACCTCACTCTTAAGTTTGGTAATGTATATATATAATTTTTAAAATGC
ACTAGCCACTTCTATACATTTTTTAAAAATACATAGGTTAACACTGCTATAAGTTTAGCAAAACATACCTGCATATTTCAGGGTCCAGCTGTCT
TTGTACCTTTTGATACACCAATTTTAGTAAATTGATCATGAGTTTGTTTGGAAGGTGAAACTTTGCTTCTTGATGGTTTTGTTTGTTTTACTTG
TAATGTTTATCTACCTTGGTATATTTGACTAAGCCTATTGGCAAAATGTAATTATAAAATAGTATATTTGACAGTTATATAATACTCTATATAC
TTTTTTAGTTTGGGACAGGTTTTCCCATAATCCAGGGTAGCCTAAAAGTTGCTAGGTAACCAAGGTACCACTTGCCTCCATCTCCTAGTGCTAG
AATTAAAGGCATGTCACCATGTGCTCTATTTTTCTATTAACAATTCTCTTAGGCAGGCCTATGTTTGTTTCTCTTGTATCTTCTTTATTATCAC
AGTCAGAAACAGGATCTGTGACCTAGAGGTTGGTGTGGCAGAGAGCTGACTTTCTGAGTATGTTTGATTTTTGTCATACTTTTGTTTAACTATG
ATGTGATTTTGAAAGTTTCATTTTTTGCTTTTAGTTTTCAAAATTTGGGTATTCGGTGTGTAAAGAAAAAGGAAGTGAAAGAAGCTATTATTTT
AAGGATTAGTGCAGGAATCAATCCTTTCAATGGTAAGTGCTTTGTTATAATTTCTACTATTTGTATTATCAGTGGACTGTGATTTCTATTTATA
GTATAATTCTACTTGTTGACTTGTATTTAAGTGGAAGCCTCCCCTCCCTTGTTGTTTATTTTGACGTGAGGTCTTTATATGTAGCTCTGGCTGC
TCTGTTACTCACTACGTACTCCAGATTGGCCATGAACTCCTAGCAATCCATCCTCATTGGCCCCCAGGTTCTGGGATTGTAGATGTGAGCCACC
ATGCTCAGCTAAAACAGAAAACCCCAAAACTGAAACCTTCTCCAAGTGTTTTTGATTCTTTCAATATGCTTCTTCTTTATGCCAGTCTACCAAC
CATCAGGATCACCCTTCTTTCTGAGTGTTGGATGGACACTATGTTATAAAGTATTTTCCTCCTTTACCACCACTGTTATTTAATAGTGATCTCA
TACAGCCCAGCCAGCTTGAATTGCTGCTTTGCTGAGGCCGGCCTTGGGCTCTTGATCTTCACCTTCTAAGTTGTCATTACAGGCAAGAGCTCCA
TACCTGGCCACTCAAAATATTTTTTAGACTTCTCTTGATTTTTTGTATGAATGTTTTGCTTGCATGTATGTATGTGTACCACAAATATAGCTGG
AGGTCAGATCTCTCAGAACTTGAGTTGCAAATGTATGTCACATCTGCAAGATGTGACTTGGAATTAAACCCAGGTCCTCTGCAAGAACAACAAG
TACTCTTAGGTACTAAGGCATCTCTTCAGCCCTTCAAAATATTTTTGTAGTCTGTTTTTGTATTGGTAAAATAAATCTTACTGTTTAGTACATT
TTATTGTGAATAGCACTTACTAAGTGGTTTGTTTTTATTTTATTGTTTTGGGGACAAAGCCTTACCACATTAAAAAAGGCTAATTTCAGATGCC
TTGTCCTCCTATCTCAAGTCTCCCTCCTGCTGGGGTTATAGACACATACCACCATTTATAGTTTAAGCATTTGATGTTTGTTGTTGTTTTTGTT
TCTGTTTCTGCTTTTGAGACAGGGTCTCATGTATTCTGCACTGGTCTCAAACTCACTATGTAGCCAAGAATGACCTTGAATTTTCTGATCCCCT
GCTCCACCTCCCAGTACTGGGATCCTTCACATTCATCATAACACTTGGCTTATGAATCATTGGAGATTAAACTCAAGGTTTTGTGTGTGCTAAA
GAAGCACTCTGCCAAATGAGCTGCTTCCTTGGCCTTTATGACACTCTTGGCTATGGCATCTGGTACCTTTAGCATTCTATATTGTTAAGTAGGT
AAATGATTCCTAAGTGTATGCAGTTATTCTATGTACCTATTTTAGTAGTTTTGTTTGCCTAGGCATGGTAGAACACACGTGTAATTCAAGCTAC
TTGAGATTGCATCCTTAAACTTACTGTGTAGCTAGGGGTAACCTTGAATTCTGTTTGAGCCTTTAGTCATTTGAAGGAAGGTAATAACCAATTG
TATGTATCCTATCTTCAGTGTTTAGTGAAAATACAAAGATGTAGCAGATACACCCATTCTGAGTGCATAAGACTTTGCCTGATAGGATAGAAGT
GGCACTTGTAGGTGACAGTCATGTAAGATTAAAACCTAAGTACAGGGGGCTGGTGAGATGGCTCAGCGGTTAAGAGCACTGACTGATCTTCCCG
GGGTCCTGAGTTCAAATCCCAGCAACCACATGGTGGCTCACAACCATCTGTAATGAGATCTGAAGACAGCTAAAGTGTACTTACATATAATAAA
TAAATAAAATCTTAAAAAAAAAAAAACCTAAGTACAGATCTGGTGCTGCGCTGACTGAGAGAGAAGGGATATTTTTTTGTATTCCAGGATAAAG
TTTAGGCTTTATAGAAAATTAGTTCCGGGTAGGGAATATGACCAAGTAAGTGAAGTAAGCATGAAGATCTGAGTTCAATCTCAGCATCCCTTTG
ACAAGAGCTAGGTATTCTCAACACCAGGGAGGTAGCTCCAGGTGGAGTTCTGGAGCTGGCAAGGCTCAGTGGGAGACCCTGTCTCAAAAATACT
TAGTGCTGATACTTAGTGCTGATCTCTACGTCCTCACGTATGCTTATCTGTACATACTGTATACACACACAAAGCAGTTATTAATTGAATTTTC
AGCTTTTACTCTGATCCAGATATCCCTTGTGATTCCTGGTTGTCAGTAGTTTTTAACCATGTCGTACAGAGATGGGAATAATATTGATAAAATA
AAAACTGGTTTTCTACATACTGAAAAAATTCAAAGCAAAATATCCCAAATTATAATCTTTTGTGAAAATAGAATTTCTTGTCTTTGATAGAATC
ATGGGATGCTAAATACATTTATATTTTAGTTTATTAGCCATTTTATTTTTCTTTAATAAATCATTTTACTACTTTAAACTCTTATACTTGAATA
AATCCACCCAGATACAGTCTTTCATGTAACCTCAAGGCAAAGAAACATTTCCCCCACTCCCAAATTGTCTTTGATCTTTCTCAATCATTTCTTT
CCTTCCAGCTCTTGCCCCAGTCAACAGCTCATCTCCTTTTTGTCAGCACACCTAATTTTTCTAGGGTTTTATAGAAATGGAAGTATTCTTTTAT
ATTCTATATTCTTTCCTGCCTGGCTCCACTTGGTCTTACTGGTTTTGCAGTGTATATTGTTGGAAAATATAACCATAGTTTGTTCTTTATTGCT
GAGTATTTGTTCATTGTATGACTATACTACAACTTGTTTCTATTAACAGAAATTTATTTGTAAACTGTCAGGAGCACCTAGCCATTCATTGTTT
TTGTCTGTCTGTCTTTCTGTCTGTCTGTTTTGTTTTAATATTGATTACTCACTTTGAAATGAAGACACTGAAACCTTGAGCTCTTAAATTTATA
TTGCTATTAAATGAGACTTTCAGTAGTCCTGCACTCTTACTGCCTGTCCTGGTCCCAGCAACTGTGCTACTGTGATACAGGTGCTGCCCTCTTA
GCATTTTGAACCTCATATTTTCCCCTTGAACATGGTATGAGGGTGCGTACCTAGGTATGGAGGCAAAAGGACTGCCTCAGATGTTTGCCTCCAA
CATTTTCTGCCTCCCTTTTTTGAACTGGGGTTCTCATCTGCGGGCTTAGATGGCGACCAGTACCCTGCTTCTGCCTTCCTGGCACTGAGAATAT
TATCAGGAGCCTGACTACTACTGCACCTGGCTTCATTGCATGGGTTGGAGGGTTGAGCTCATACTTGCAAGGAAAGTACTTTACCGACTGAATC
ATCTCTCTCAGCCCCATTTTGACCTTTCTAGTTGTACAAGTATGTTTTCATAGACTCAAATGTTAGGCTTAAGAATGCTGATACTGGAATAAAT
TTCAGTGTTAGCATAACTCCTGAGACTATTTGACTGGCTTTGTGTGAGATATGTTTTTTTAAATAAGTTATCACCTGTAAGACTGGGGAATTTA
GCTAATTATTTTTCATATTTAATCTTTTAATTTTAATACTAGTGTCCTTTCCAAGTAAATTTTTCATGTAATTTAATGAATTTTATGTTTTTAA
AATATGGCATATATTAGTAACAATTTATTGCTATAGGCTTAAAATATTTCAAAACGCAGCAATATTTTVTTTTAGAAATGGGCTTCTTTTCAAA
TATCAATGCAAGAGACTATTGTACTATATTCTGTGGCAAGTTTTTGTATTTTTTTTTTCAGAACTGTAAAAATGCCCTGGTGTGCAGTTTGTTT
TTAAGCTGACTTGACTTACACATAAAGTTATATCATCTGATATCTTTTTCACATTGCATCTCTAAAATGCTGGTGCAATATGTTAATAAGTTAA
TATGACCCTCTGCTTCAAATTTCCATGCTATTATTTAGCTGTAAAATTTATTATTTAGACAAATAATGTCATCTCCTTAAAATCAATGTCTTAC
CTATAAGAACAGGGATGATGGTGATTTTTGCTAGATTTAGAACGTAATTACAGACTGGGCCAAGTGTTATGTTAAATGAAGTATGCATGTTTCC
CATTGTCTATAACTTGTATCCATATTTAAGATTTTTGATTTTGGTGGTCAGTTTATGATTCTGCCCTTAAGTTCCTAGTCCCTGGTATAGTCTC
TCTCTGAGCATGGCTACAGTCTGTTATATACTGTTAAATAGTAACTGTACAATAATGAAAACTGTTCTTGAGATTGAACCCCATCTTAGTTTTC
TGCAGACGTAGGTGCGGGTTAAGGTTATTGGATGTTGTGGGCAGAGAGAAAGACAGCAGGATTTTGGTAATAATAAAGTATTAGTATTATGTGT
TCTACATTCTAAATCTGAGATTGTACTGTTCAGTCCCTGAACAGCAGCTGCTGGACATTGAAGACTGCGACCTCAATGTGGTGAGGCTGTGTTT
TCAAGTTTTCCTTCCTGATGAACATGGTAACTTCACAACTGCTCTGCCTCCCATTGTTTCTAACCCAATTTATGACAACCGTAAGTGCTTTAAT
TTTCTTATGTCTGTACTTTTAGCTTGTTTCATTTCCTTAACTGCTGAAAAGTTTAAAGGATTTTTGCAATTTGAAAAGGAGTATGTTAGGTGGG
ATTGGTGGTTTGTGCATATAATTCTAGCAACCAGAAGGCTGAGGCAGGAGGATAAAGTTTGAAGGCATAATGGACTACACATTGAGACCTTGTC
TCAAGAAACAAGAAGAAGCCAGGGGTGGCGGTGCATGTCTTTAATCCCAGTACTCAGAGATAGAGATAGGCAGATCCCTGTGGGTTCAAGGCCA
TCCTTGTCTACCCTGTGATTTCCAGGATTACATAAAAAGATCCTGTCTCAGACAAAAAACAAACAAATAAACAAAGCACTGTTCATTATAGAAA
AAAAAACCCTCAAGCATATATACATATATAAGAAAGAAGAGCAAATCACCATTAGTCTCCTCCTAAATGGCCACTGTCCTCAAGCATACACTGT
AGTCCCTTTGAATAAGCACAGACACCACCATCTTCCTCATCAACCCCATTCACCCACTAAGTTTGGCAACTTAAAAAAAAAGATCAATGATATT
TTAAAAAAAAACAACTTTTTTTCAAGCCAGTAGTGGTAGTGCATGCCTATAATAATAGCACTTGGGAAGTCCAAGCAGGCAGATCAGGAGTTAA
GTTTTCCTTTACCATGTTTAAAAATGGGAATTCAAGACCCTACTGTGTCACATGGGATCCTTCCTCAATAGATAGAGACAGAAAATCCCAGAAC
TTTTCTCTTTGCAATGTATCTTGAACCTTTTTCAATCACTTTTTCTTTTCTTTTTTCTTTTTAAACACTGCTGGCATTTAACCAGGACATTGTA
CAGAGCTATACCCCAAACCCCACTTAGGTTTCCAGTAGCTGAATATTTATCTCCTTCCTTCCTTCCTTCCTTCCTTCCTTCCTTCCTTCCTTCC
TTCCTTCCTTCTTCCTTCCTTCTTCTTCCTTTCTTTGTGTTGCTTTATACTTTTTTCTTTTTCTTTTTTCTCAAGAAAGGGTTTCTCTGTGTAT
CAGTCCTGGTTGTCCTAAACTCACTGTAGATCAGGCTGGCCTTGAACCACAGAGATCCACCTGCCTCTACTTTTGCCTCTGCCTCCCAAGTGCT
GGTATTAAAGGCCAGTGTCACCTTGTTCAGAAGCATATTTTTTTATATGTCTATATATTAATTTTAAGAAATCATTAATATGGAGTATAAAAAA
TAAAAAAGATGACTTTTAAGAATCATATTTAAGAATATATTTGGTTTGTTTCCTTTTGTAAGCAAATTATACCAAAGTAGATATATTTATGTGT
GCATTTAAAGACAACTCTCTATGGATCATGTATCTGCCTGTTTATATGTGTGTGGTCAGAAGACAACTTACAGGACTTGGCTGTGCTCTTTCAT
CATGTGTTTCCCTGAGATCAAACTCAAGTTGTCAGGCTTTGCTGCCTTAACTATGTCACTGGCTCCTGAATGTTTATCTCTTATGCAAATATTT
CTGACATTTATTTTTAAAAGAATAACTTTTGACAGATACCTACATCAAAGGATATAGAAGTTTATTTGAAAAGATTTTAAAACTTTCCTCCAAA
ATATTTTGTGTATATACACACACATTCACCAGTAGTATATGCCACACCTGATTTGCATGTAGAATACTGGACCATAGGAGTATAGAGGTAGATT
ATTCTTAACAGAGAGCCTTGGGTACCTTTAAGAACTATCATAATAATATTCGGAAGATAAATGAGAACTGCATTGCCCTAGGGATATAGCCAGT
GATATAAAATGACTCATTTATTCACAGTGTGCCTTTACCAAGAAACAGGAGGAAGAGGCAAGAACTGCTCAAGTTAAAAGGCCAACTAGCCTGG
TGTACATAGCCATAAGTAACAAAGAGACTGTCTCAAAAGAGGTAGGAGTTGAGGGCTAGCAAGCACCTGAAGTTGTCCTCTGACCTCTTCCACA
CATATGCTGTGACATATGCTCACCTGGGCCTACATATAACCACACTCAGACATACTATAGTTATAATACATACATAACCACACTCAGACATACC
TCAGCATAGCTATAATACACACATAACTACACTCAGACATACCTCAGCATAGCTATAATACACACATAACTACACTCAGACATACCACAACATA
GCTATAATACACACAAAAAACATTTAGTTTCCTTTATCTATACATTAAAATTTTATCTATTCTCTAAATTATTTTCCTGAAATTCTTCCCCTTG
TACTTGTACTGGCTAGTTTTATGTCAACTGACACAAACTAGAGTCATCAGAGAGGGAACCTCAATTGAGAAATTCTCCTTGGAAGATCTGGATA
TAGGAAAACCTGTGGGGCATGTTCTTAATTAGTGACTCACAGGGGAGGGCCAAGTCCATGGTGGGTAGGGCCGCCCCTGGGCTGGTAGTTCTGG
GTTCTGTAAGAAAGCAGGCTGAGCAAGCCATGAAGAGCACTCCACTAAACAGCACCCATCCAGGGCCTCTGGATCGGTTCCGGCCTCCAGATTC
CTGCCCTCACTGCTCGTTTTCTCTTTTGATTTATTTGTTTTATTTATATCAGTACACAGTTGCTCTTTTCAGACATGCCAAAAAAAAAAAAAAA
TCATCCCGTTACAGATGATTGTAAACCATCATGTGGTCACTGGGAATTGAACTCAGGACCTATGGAAGAGCATCTGAACCCTCTCTCCCATCCA
CCCTCACTGCTTCTGATGATGCACTGTTACATGGAACTATGAGGGAAATAACCCTTTTCTCCTCAGGCGACTTTTGGTCATGGTGTTTCATCAC
AACAACTAACATTAACTAAGACAATGCCGAGACCCAATGTTTTTTTTTTTAATCCTTTGCATTTAGGTGCCCCAAATACTGCAGAATTAAGGAT
CTGCCGTGTGAACAAGAACTGTGGAAGTGTCAGGGGAGGAGATGAAATATTTCTGCTTTGTGACAAAGTTCAGAAAGGTATTTATCAGCTTTGT
TTAATTGAATTAGTAGTACATATTTGATTAATATATGTATTGTACCGTTTCCCCATTGTTATTATTCTTTGGCATCTTATTCACTAGATGATAT
AGAAGTTCGTTTTGTGTTGAATGACTGGGAAGCCAGAGGTGTTTTTTCACAAGCTGATGTACACCGCCAAGTAGCCATTGTTTTCAAGACTCCT
CCGTATTGCAAAGCTATACTGGAGCCTGTGACAGTGAAAATGCAGCTGCGGAGGCCTTCTGACCAGGAAGTTAGTGAGTCTATGGATTTCAGAT
ACCTGCCAGATGAAAAAGGTATGGCAAGTTGGGAGTAATGTGTATACGTGTGTGTGTGTGTGTGTGTGTGTGTGTGTGTGCTCACTAGAAACAG
CTATGTAGCTACAGATGGATCTTAGCTGAGTGTGGGGATTACAGCAGTGTGCCTTGTGCTGTCCCTGCTGCCTTTTTAAATGTGCTGCTAGGAA
TTGGACTTAGACTTTTATGTACACTAGCAAGTGATGAGCTGACCAAGTGACAGTCTCTGCTGAAGGACAGATCGTCTAAAGCTGATCATGCTTG
TAATATTGTAAAATAAACATTCGAGTGCAGTTTTATATCTTCAGAATCTGTATTTTTTTTCCCCTAGAAGTTTTCACTTGTGTGTTTTGGTTTG
GTTTGGGTTTTTTTTTTTTCTCCTACTCTTTGCATTTCTTAAGAGTTTGAATAATTTTAGAGTTCTTTTTTTTTTTTTTTTTTTTTTTTTGGTT
TTCCGAGACAGGGTTTCTCTGTGTAGCCCTGGCTGTCCTGGAACTCACTCTGTAGACCAGGCTGGCCTCGAACTCAGAAATCCACCTGCCTCCC
GAGTGCTGTTTCTCAACTGTTAGATTATATTTTCCATCTATATATGTATTTTTGAATTTATTTTCTGTACTTTTTATACCCTTTTATAGGTGGG
AAATTTGGTAATCATAAAGTTGTATCTATTTTGGTCCAATGTGTTAGTTCATGTTGACTTACAGTTATTTTTAGGATGAAAAATTATTATTTTA
AGCTTTTTAAAATTAAGAGTAAAGTGTCCTTTATAACTGGTACTGGATGTATTGTACAGTAGCATACTTCCAGCCTGTACTGTTCGCATGTATT
TTAAGCATTGCCACACTCTGATAAGGCGCTTGTTCCTGGTGCAGGAAGGAAGAATCCCATGACTTACCGCTTTCCCACTTTTCTTTCTTAATCA
GATGCATATGGCAATAAATCAAAGAAACAAAAAACAACTCTGATTTTTCAGAAACTGCTGCAAGATTGTGGTAAGAATATTTTGGATTGAGTTA
TAGTTAAGTAAATGTGTCTTATTTGCTTTATTCTGTTTTTAAATTTTTTAAGTGATAAGCTGTTTTTCAAATTACACTTAATAATAGAAAAACT
TATCATACCAGGTCACTTTACTGAGAAACCAAGAACTGCCCCTCTGGGATCAACTGGAGAAGGAAGATTCATCAAAAAAGGTATTTTATCCACT
TTTTAGGAATAACTGCTTTTTAAGTCTCTTTCTTTCCTACAATCCCGCACACTCTCACTATTGAGTGCAGCTAGGCTGTACCTCTGCAGGTTGT
GTTGGAACTAATTATATTTGAGGGATTTTATTGTTCAAGAAATTACATTTTGTCCACACACTGGTGCTTTTGCTTTTTTTTTTTTTTTTTTTTT
TTTTTTTTTTTGGTTTTTCGAGACAGGGTTTCTTTGTGTAGCCCTGGCTGTCCTAGAACTCACTCTGTAGACCAGGCTGGCCTCAAACTCAGAA
ATCCGCCTGTCTCTGCCTCCCGAGTGCTGGGATTAAAGGCGTGTGCAACCACTGCCTGGCTCATTGGTGCTTTTACTAATTTGACTGTTACCAT
GTAAGAAATGGCATGGATGTGGTAGGCACCTGTTTATAGCTGCTTACACTTTGAACAAGGCTGTGGCCCTTGGGGTCATGTACTGTCTCTCAGG
GCCACTTTAAAAGGGGTAGGACCACAGGCATGCTGACCACCTGCTGTCATGCCCAGACCTAGAGTCCCCTCTCTCTTCTGCTACAGCAACAGAG
CTGGACAGTTCCCACAGAGATTGCTTCCCACATAAACCTTAAAACTGTCTGGCCTTTTAGAGAGGATATTTTGCAATCCCCTAACAAACTCTCT
GTCTTGTACCCTTACATTGGTGCTTTATCCTGGTTTCTGTAGGGCTTGTGTAATATGTACATAAATGCGGGCACTGTCCCTCAGTTGTCTGTGG
TGGGGAATTTTGTGAGACCTTAGGTCATGTCTTAAGTGATAAAAGCTATGTGCAGTTGTCAGTCCTCTCCTTCTTCCCATTCTCCCTAAAAGCA
GAGACTAGAAGATCATTTTGGCCACTAGGTACCTGTTTGTTGAAATTCTAATATTTAAAAATGTGCCTATATGGCACAAGAAGCTGGATTTTCA
TATTTAACTGGAAGAAAATTACACATGCCAAAATAACTTTGAAAAACATTTTCATATATTTATATGGAAATTTGCATGTTACAGACTTTAGTTT
TGAATTCAAGTAATAATTTTATGCTTTAATTTATTTGATATGTGTCAGTGCCCTAGAAAGTTTTTAGTATGTTTTTATAATTTCAATTCAGAAA
TAATGCTTTATATCAATTTATTCATCTTACTCTATAAAATATATTTTTCCTCTACAGAATCAAACTTATTTTCTCATGGTACAGTTTTTACCAG
AAATGCCCAGGTCTTCAGGAGTTCCAGGGCAAGCTGAACCTTACTATTCTTCATGCGGGTCCATCTCGAGTGGATTGCCACATCACCCACCAGC
CATACCCTCAGTGGCCCATCAGCCTACAAGCTGGCCACCAGTGACTCACCCCACCTCACACCCAGTCAGTACAAATACCCTGAGTACTTTCTCA
GCGGGAACACTTTCCTCTAATTCACAAGGTATCCTTCCATTCCTAGAAGGGCCTGGTGTCAGCGACTTGAGTGCATCTAATTCGTGCCTTTATA
ATCCTGATGACCTAGCTAGAATGGAAACGCCATCCATGTCACCAACTGACTTATACAGTATCTCTGATGTCAACATGCTATCTACTCGGCCTCT
GAGTGTGATGGCACCCAGCACTGATGGCATGGGGGACACTGACAACCCAAGACTTGTGAGCATCAATCTTGAAAACCCCTCATGTAATGCCAGG
CTAGGCCCAAGAGACCTGAGACAACTACATCAGATGTCTCCTGCCAGTCTGTCCGCCGGGCACCAGCTCCAGTTCTGTTTTGTTTCACAGTCAG
ATGCATTTGATAGATCAAACTTCAGTTGTGTAGATAATGGCCTGATGAATGAGCCTGGACTATCAGATGACGCAAATAATCCTACCTTTGTTCA
GAGCAGTCACTATTCAAGTGTTAACACTCTGCAAAGTGAGCAACTGAGTGATCCCTTCACATATGGTTTTTTTAAAATATAAGTTGTAGGACTT
AAATAAGTATATAGAATATTAAGAGAAAGTTCAGAAGTCAAAACTTTAAAAGCCGCTACTGTTTCTTACTTTGAAAGATATGCCTAAAGTCCAC
GTGCTTGCTGTGAGTTGTTTTCTGTATTTCCCCAAGACAAACTATGTATCTTTGCTAATGTATTAGTTAAATGAGATGATATTTGCTTTCAAGC
AGATTTAAGGTGAAAAACGTGTAATGGCTATGCCATTGGGAAATAAACTCATTATTATTGTTGAGGCCCATAGGCCAAAGTGACCCCTAAGGGG
TTTTCTGAGGTTTCTTGGTATTTTTAGGGTGTGTGTGTGTTAAAGTAAGCTTTCAAGGCATGGAAGTTTTTTTTTTTTTCTATTTTCTATTATT
TAGGCTTTTATCTTTAAAGTTACATGTGAAATTAGCTTGATATCTGTATTTTCAAGAAAAGTTTTCTTGAGCCTTTTCTTTTTAGATGTTCTCA
TTCCGTTTGATCTCCTAAAGCCTGTCTGCTTCACTATAAAGAATCTAACCAGGTATTTTGGGCTCATGTCAGTAATTTCCCTACTTGAGATCGT
AAGTTCAAAACCTGCCTGAACTACATAGTAAGTTAAATACCAGCTTGAACAGCTTATGGAAATCTTGTCTCAGAAATAGAAAAGACCAAGGGTG
TGACTCAGTGGAGGAGCTTGCGTAACATTTGCTAGGCCCTGTGTTCAGCCCTATGAAGAAGAAAACAAAATAACAAGTTCGTTTTAGCTCAGTA
TATCCATACAGAGTTTTGAACTTCTCTCCCAGCCTTGTGTCTCCTTCAGCCTTCCCTGCTCTGTATATTGTCACGCCATGCTTCCCGTTGCTAA
GCACCTTGGTGCGCCTTTATTCTTCCTTTACCTCCCACATCTGTCTATTCCATCGTCAAACCCAGAAGGCCATTTCCTCAGTATGCATCCGAAG
CCCACCGTCTCCTTTGAACTTTTGTGTGAGACAGGCTGGCCTCAACCTGCTATGTAGCCAACGATGAACTCAGCCTTCAGTTCTCCTGCTCTGC
CTCGGGAGTGCTGGGATTACTAGTGTGCACCCCCCCCCCCACACACACACAGAGGTTTGTGTGGTGCTAGAGACAAAAACAGGTCTTTCCTGGG
AGAAGTATTCTCACAGTTGAGCTATCTGCTCAGCTCAGACCAACCACAAGCATTTTGTATTAGTAAAACTGATAAACCCAGACGCAGCTATTAG
AGAGCTCAACAGATGGTACTCACACAGTGACTTATTTCACAGTTTTTCCTCTAAGTTGAACTAAGGTCTGTCTACATCTGCATTTAGAGTATTT
TCAATTGACGTTTTATTTTCCAGTGCCTCTTACTGATCACATCATTCAGTTTATACCTTACAATGCATTTAGCAACATGTTTTTCCTTTCTCTA
TGGCTGCGTTTGTATTTCACATGGTTTTGAAAGAATTGGCCTTCTTTTCCAGATTCTGCTTGTGCACAGCGTCTCTCCTCCTGTCCCCATGTCA
CCAGTTAGTTCTTGCTTCCTTTGCATAAACAGAGTAAGTATCTTCTTGAGCTTAAAAGGAAAGGTTAAAAAATTTCCCCTCTAAATACCAAGAG
CCCTTCCCCCACAGTGCCCTGTCCTCCACATTTTGCCTCTTCCATCTTCTCACCTCTCGCTCGCTCCTTCACCTTCCATCTCTGTCTTTAGTTT
TCCCAGTTCCTTGTCTTCCTGTCAGCCTTTACCCTCCTTGTCTGTGCTGTCCATAGCCTGGCACCTCCTGAAGGACAGGATCCTGTGGGACGGG
GCTGTGTTGTCTGCACTCTCCTCAGCACCGCAGCTTCTCCTGCTGTGGGATGCTCTTGGTAAGCAGCTGCNNNNNNNNNNNNNNNNNNNNNNNN
NNNNNNNNNNNNNNNNNNNNNNNCTGGAAATCTTTATTCTTTTTCTTCAGTGAATATCTTTTCCATATGTTTAAGGTTTTTTTTTTTTTGATAC
CTTATTCCTCTTAGACGTTGTAGTTGGAACATGTTGAATTTATTCCCACAGGTGTCTATAGATGTAACCTATTCTGCCTGTGCTCCCAACACTT
AGCTTTTAACAGCAGGCTTCTCTGCAAGGCTTGGTCCTGCTGCTACAGCTGAGAGGTGCAGCTCTGCCTCTCTCTGCTCCTATATTACAGACCT
TCAGCGGATGGTAGTCAGTTAAGTGCTTCTCACTTATCTCTGGGACTCTAGCATTTGACGTACACTCTGGCCTAACTGGTGCTGTATAAATAGT
AGCTATTTATTAGAAAAGGGGGATAATTATAAAATCATATTTGAGGATATAGTTTTTAATGGGAAAGGTGCTTCACCCTGCAGGAAAAGTAATG
ATGCCAAGATAACCTGTCCTGTGAGACATACTTCTCTTCTGGTCGACTGCTGCTCTTCCTCCTCCTCTTCTTCCCCCTCCTCCTTCTTCTTCCT
CTTTCCTCCTCCTTCAAAAAACTATACAGACACTGGCACATGCTTCATGGTAGAGCATGGCCCTGTGTTTGATACCCAGAATTGCAACAACAAC
AACAAAATTATAAAACAAAAGTATAAAAACTACCCTGAGGCTCTTTGATACCCAAAGCTGTTCTCTTGGAAATCGTAGTACCACCAGTATAGTA
TCAGGTAGAAATCCTGCCTCTGTATGGAAGGTTATATCCTACATACCCCAGCGGTACCCCTTGACAGTTTACCATGCATAGACATGTAAGTAGC
CTGTATTCATAACATTTTTGTAACTGTATATTGTACAATGTACCCTGTTCCATGTAGAAAATAAGAGGGGAATGGTATTCAACTTTTGTTCTCA
AAGATATGTTTCTTTTCTTATGAAGAATCACATATATAGTCAACCACAGAAACATGGACAAACTTTAAGGTATAATTTGAAGAATTTTTGATTA
TATTAAAACAATGCAACCACTATGCTTGGGACCCCAGAAGTTCTCCGCACCTCCAAACCACTGTTCTGATTTTTGAGAGCCTGCTGACTTCCTA
TCCTTTTCTTTTGCTAATTAGTTCTCCATTTGGAAAACCTTAGTGTAGAGTAAGTCAGTTTGTTTTACTATGCTGTGTCGTGTTTCCACAGTCT
CAGAAAAACTATATTTTTGTGGAGAGACTTAAAATTGGTGATCCAGGTTGTCTAATTGTACCATATCAAGTTTTAATCATAAGTTGGAATATTG
AAAGTTTACCATAAGCTGTCAGACAGCATGCAACAAGATAAAGAAGAGAGACTGATTTTAGTCAGTTAGCCTAAATGGACCTGCTGGTCTTCAG
AAACTCAGGCAGAAAATCACAAGTTTAAGGCCTGCACAGTCTGCAGAGTGACTTCAAGGGAACAACTTTGTGAGCTCCTATCTCAAAATATAAA
GTGGAAAAAAGGGCGTCCTGAGCTATAGTTCAGTGATAGAGCACCTAGCATGTACAGCTAAAGACTCGGTGCCTAACTCCAAAGAACTAAAAAC
CTGGCCTTCCTACTTCCTAGGGTGTGGTTTGTGCTAAGAAATGTCTGAATAGAAACACCGAGTTAAAGATTGTGTTTCTCATGGGCCCTGATTA
GTCTATTATCCAAACATTCTTAACCACTTGATACCCAGAGAGAAAAATCACAGGGTGGATAAACACTTTGGGAGACCTATGAGAAAAGCAGGTT
TCTCTTTAGACAAACGTCTTTAAGTTGCCCCATATGGACCTGGCTCTCTCCGAGGACACTTTCTTATCAAGGGAAGTCAGTTACTGGCTTTACT
TTTGGTATTTTGTGACTTGATTTCTTTGTGTAAGTTGAGAGTCTTTGGGAGTACTTTAACTCCTGTGACGTTGCCACTGCAGGGTTTCAGGTCT
GTTCAGAGGTTGGATGTGCACGGAAGAGGATAGAATGCTTTTGTGTCTAGGAATGGAAAGCCCACGCTATTCTTGGAAGCCCAGCAGTGGGCCA
TAGGCATGAAGGTGCCACCCAAGTCTATCCAGTTTCATCCCTTGAATATTTAAGCCGAACATCATGGCAGAATTTAAACCTCAAGGTCAACAGT
GGATGAGATCAAATCTCAAAAGTGATCAGGTCTGATTGTTTTCATGTCTGTAGAAAGGCCGAAACCCACGCATGTCCTCATCATCTTTTGTCTT
TTTGGTTGGCTTTGGGGAGGTTGGGTTTGGTTTGGTTTGGTTTGGTTTGGTTTGGTTTGGTTTGGTTTGGTTTGGTTTGGTTTGGTTTTTTTCC
CAAACAGGGTTTCTCCGTGTTGCCCTGGCTGTCCTGGAACTCATGGAAGTCTGCTGCCTCCCAGACTTTATTTATTTTTTTTTTTAGCTTTCAG
AACTTTAAAGAATCACAAACAAGCCAGGTGGCACTAACACACTCCTTTAAAACCAGCACCCAGGGGGCAGAGGCAGGCAGATTTCTGTTAGTTT
GGGCTGGACACTTACTCTGTAGTAAGTGTCAGGCCAGATGGGGATACACAGAGAAAACTTGTCTCAAAAAACAACAAACCCAAAAACAAACCAA
TACAACAACAAAAGAATCACAAACAAAATAAAAAGAAATGTTGACTCCTCAACAAATTGAGTTTTTAGCAGCTTTATAATTTTTGGTAAAATAC
CAAATTTACCTATGGTAATTTTTTTCTAAGCTTCTTTAGCCAAAATGTAACTTCTCATGAGCTGGAGTAAAGTAAGGCTGAGGCATCTTGGGAT
AACTATGACTTCACTGCTCTTAGTGAAGCAGCCATAGTGATATATCCTGTAGCTCCAATACTTGGAAGGATGAGGCAGGAGGGATGCTTGAGGG
AGACCTCAGCTAGTCAGTCACCAGAGAACTTTAAAACACTATTGGCTATGGTTAATAGACCCCTACAAAACAGGGGAATCTTTGCTGTTGTTTT
GACCTTAAATTGAATATTGACCTGCCTCTCCCTTCTGAGTACTAGGATTGTAGGCATATGTCACCACACCTCACCTAATGTAAAATATTTAGAT
TTCAGAGAGGAGGGGAGCATCATGCTGGGATTGAACCCAGGGTCTCATACAAGTTATGGTTGGTGTTAGCTCTCTACCACTAAGTTGTCTGCAT
TTCAAACTGATTTTATATGCTTGATGATGAATGAATTTCAAGGCTCTGATGAAATTATTTTTTTTTAAGTTCTCAAGATACTGGTACTTAAGAA
GTTAAGCTTTGTATCAAGATGAAATATTTTTTTAATGTCAAGATGCCTTTTATTATATAGCATATTGTGTTACTTTTGTAATATCTTTGTATAC
TGTGATTTTTGTTGATGTAATAAATAGACCATTTTCTAAAAATGCTTGTTACAGTTTTGCTGTTTGCTTACGAAATATCAGATTTCTTTTTTTT
TTTGTTTTTTGTTTTTTACTTTTTCATTATAAGGGGTAGCTATTTGGGATTGTTTAGGAATAGTAGGCATTTTTATAAAATGTTCTCAGGATAC
AGTCTATCTGTAACTACCCTTTGAAATAGTAGTCTCAAGAAGTACTTGAAATGCCCTTTCAAGGAGTCTTGGGTTGGTTAGATGGTTAGTTGGT
TGGTTTTTGAGATAGGATTTCACTGGGGTTTTGGGCATGAACCACCATGCTGTTTTAACAAGGCACTGAGCATGGAATCTAGGGACTCATGCAT
GCTAAGCAAGCATTCTGCTGCCTGAGCTACATCTTCAGTCCTTGCAGGCTTTGGTCTTTATTATTTTGGAACAGGGTCAAGCTGGCCTAGATCT
TGCTGCATACTTCAAATTCATGGTCTTCCCACCTCTGCCATCTGAGTCCTAGGATTTCAGGTTGAGTCACCATTCCTGCCTCTCCAACTTCATT
CTGGTTTAAGCTCACTTCAATTGCTTGGTTTTTCTTCTGATTCCATACATATCTTTGATGGGTCTGCTAAAAATATCATTGGAATTATGATTGC
TTTTGTCATATTAACCCATCCTTGAAGTAATCCCACATGGTCACAGTGAGCTACCTTTTGAGTATGCTGTTCAGTTTGCCTTGATAGCATCTTA
AATTTTTTCATGTATGTTCATCAGGGGTACTGGCCTGAGTTTTGTCTTAATGATGTCTTTGTTGGATTCTGCCATCAGAATTTGGCAGCAATTC
CTCTCTGCTTGCTTTAGAAGTCTTGCTGCCAAGCCTGATGACCCAATTTACATCCCCAGAAACCACATGGTATTCAAGAACTAAACTTACTCCC
ACAAATTATCCTGACTTCCTTACAAATGCTGTATCACATATGGACATGCACATGATAAATATAAGAAATTTATTCTAGAAAAAATACCATTTTC
CACAAATGGAACCTGATCTTTTTAGAGAAACTGCCTATTTTGTGGCTAGTGTAGAGGAAATACAAAGCCTGGGATATTTTTATTTTACAAGAAT
ATGCTCAAAGAACGATGGAGATAATGTTAGAAGGGCATAGGAATGAGCTTGAGAATTCTCACTGCTCAGAGTAATATATAGAAATAATATCAGT
GGCAAATTACCAAGTCATTGAATAAAATGAGAATCTATTGGTGACAGTGCTGCTTTGTCTTGGTTCAGTCCCTAACACCACAAAACAACAAAAA
ACAAAACAAAAAAAACACAACCCAGGCTTCAGTCCACATCCATAAAGTAAGGAAATGAGAGAAGGCCCTCTCTCTCAGTGGGATGCCATTCAAT
ACATGTGAAAAGAATGACACACATTAGCAGTTATACTTAGAATCAGTAGTAACTGAGTCAGGTAGAGATCTTTGGTGGATTCCAATCAAATGAG
TGAAAATCTGGTCTATCTGCAAGGCACTGAGTAGAGAGGGAAAGGACAGTAAGACTGGAAGAGTTTGATAGACAACACCCACCCCTCAGGGACT
AGAAAGTTTGGCAAGATGCAAGGAGGAAGCATCTCCTCCGTGGTGTCCTCACATCTTAAATCTAGTACCAGGCAAACTGGAGCTGAGCATAGCC
CACTAAATAACTGGGCTGAAGCTGTGCAACCGCTTCATGAAGCTTAGCATACACCGACTACAAAGCAGGAGGGCTCGGTCATGAAGCCAGTGAC
AGACAAACTGATGGACAACCTCAGCGATGTCTTCCGACTAGATAATGGTGGTCTGTCAGAGCTCACTTCCTGGTATGGTGGGTGGCCTGTGATT
TACAAGATGTCTTCTAGGAATTATGCACTATGTATGTATTTGAAATGGTGAGGTGGTGCTGACTCAGCACCTCTGTGGGAAAGTTCCTTTGTAC
TTTTTAGTTAAGTTTTGAATCATTTTGAAAGGAAAAAAAAATTAAGATATGAAAGTAATTCTTTTTTCATAATAATAAAACACAGATTCTGCCA
GCTAGTTTTTATGTTAACTTTATACAGGCTAGGCTATCTGGGAAGAGGGAATCTTGCTTACTGATTGATGTGGGAGGGCCCGGCCCACTGTGGT
TAGAGCCACATGGTCAGGGCTCATGGCCCTGGATGCTATGCAAGAGCAGACTGAGCAAGCCATATAGAGCAAGCCAGTGAGTAGCACTGCTCCG
TGCCTTCTGCTTGAGTTCCTGCCCTGACTTCTGTCAGTGATGGAAGTGTAAGAGAAATAAACCCTTTCTTCCTCATGTCCATTTTAAAACCAGT
CAGAAAAGCTTACATCTGGACTGGGGGTTGTGGCTCCTGAGTATAATCCAGACACTTTGAGAGGCAGAGGAAGGAGAATCATAAATTCAAGTCT
AGTCCAAGCTACAAAGTGAAACCCTCTCTCTTGTGGCCTCCCTCACAAAGCTACTTCACTCAGTTCCTTCTCAAGTGGAGATTTAAAAATAAGA
CTCAGGAGTAGAAATAGTAGATGAGCTGGCTGATGAGGACTGCTGAGACATAACCTTCTCAACAACATCACTTGACTGTAGCAATCATGACCTC
ATGGTAACTGAAGATGCTTGAGTGGGTAGGGAGAGATCACGGGGCTCTGTGTCTCCCCAAGAACTACTAGCTGTTGATAGATGCTTGAAGAGGC
ATCATCTTAAATGTTGCTTCTGTTTGCGAGTTCACTAATGTTTTACATCCCTGCCCACAAAGGTAGTTAATACTGTAAGACCCCCGAAGCTGGG
CCTCTGCTCAAGTCCCGGGATGAGCTGGCCAGCACCCCAGTGTCCTGAGAGAAAACCACACCTGATGCAAACTGCAAGAGCCATGCAGGGCAGG
GGAGTTCAACCCAGAGCGGTGACAGTAGGGGGCTTTTAACAGCTAGCTGAGGAATTGGGAGGAGTTGGGGAGAGAGTTCTCTTCAGCATGTCCT
TGGTGAAATTTACAAGGCGGGAGTGGGGAGTGAGTTCTTTCTGAATTTTTATCTCCGTGTCCTCGGCACTGGAACGCAGGCATCTCTGGTTGTA
AAGTATAGAAACAAAAAGGCTTTTTGCTGGCTATAGAGAACAACGAGCTTCTTTCTGGCTGTATTTTTAGAGATCAGGGAAAACAAGCTTGGGG
GACGTCCAGGGGCTTTACCTTCCAGGGAGAAACGCTCTAAGTTGGGGGTCTCACAATACCAGTGGGTCACAAAACAAAGGCATTACAGTACGAA
GAGGTCTTGTAGGGAGGCCTTTTGTTGGGGGTGTTGGGGGATAAAGTAGGGGTCAATGTGCTAGTACAGGCCATGAATCCCAAGTGCCTTGGAG
GAAGGCAGAAACTTGTGAGCTCTAGGACAACCAGGACTACAGAGAGAAACCCTGTCTTGGAAACAATACAGAAAAAGGACTGGGTAAGTGTGAC
CACTGTGTTATATTCATGTATGAGACTGGCAAAAATAAATTTAAAAACAAAGAATACAGCCCCAAGTGAAAGAGAATGCTTGTTGTCTTTTAGG
AAATAGAAATTTAACTTGTTAAGTTACTTCATCTAACTTGCAAAGGTAACCAGTTATCTTACTAGGAAGATTCTCCTTTATCAAGTCTTTTTTT
CCTTGCTTTCTCTCGCTCTCCAGAGTTGGGTGGTTTTTTCCCTTCTGAAAATAAGCTACAGCTTATATCTGCAACACAGATAAGAAGAGAGAGC
TCATTCTGCAGATGACATAAACATTGCTTCTAAGTTAAACAAAGCATAGAGAAGAGGATACACTGTAAGCCAAGTGACACAAACCCACAAGACT
TCCTAGCCCTGTGTCCTCACTCCCAAAGACATTGTCTATAGCTACTGTTTACCTTTCAACAGGAAATAATTCAAGGAATGCTTTGTTGGAAGTT
CCTGACCTTCATGTTTTGGGTCCAGTACATTCAAAACTGAGCTAAAAAAAAAACAATGCTCAAAGACTTTTCCCAAATTATTTAATCTTTTAGC
AAAATTTCATCAGAAGCTGAGAAACATTACATAGAAGTCTTTAACTTTTCACTTTTCATCAGTTAGTTTCTGTTATCTCCTTTTCAATTTTCTG
ACAATGAAAAACCCTTTGCTATTATTCACACATGATGAAACAAGGTTTCAAAGGAGCAGGCTGCCCTTTCATCGCAATCAAAAACATCTTCCTG
ATATAATACATCTTTCAGAGTCAGTATGAATTGTGTGCAAAGATGAGAAACGTGAATAATCAAATGCTATTCAAATCAATGCATCAAAAAAAGT
CAAGAAGTTGCACTAGCTGATGAAAATTTTACTTAAGTCAAAATTTTATTGAGCTTCCCTTCTGGTATTTTTGATGATTAATTCTTCTGGTTCA
ATATGTTTAAGATTTCTCTTCACCATGCCATTACAATTGACAATTATGAGATCATATAAGACATACCATTAGCCATTTCTTGGGGGGGTCACTT
GATATCCAAGTTCCAGAAGCCAACCTCCTGGCTTTGTAAGCCTAACTGTCCTTCTTAGGACACTGTCACCCACAGGAGATGCAGGTGCTCCTGA
CAAGGGTTTTTAGAAAGGCTCCTGGAGACACAACATCAGCTCCAGACCTGTTATAAGAGTAACTAGGTGTGTCATTTAACTTTTGTTTCCTTGT
TTATGTAATCAGATGTTAGATAGACCACTTGAAATTAGAAAACATTTGAAATTGGAAAATAATTGAAACACCAAAATATGTTTGTATTTGTTTA
GCATTATCGAATTTTCAAATTTGTAACTCTGGGACAAAAGTTACTGAAGGCACTTGTTTCTCTTTACATAAAAGACAAAAGAGGTAGGCAGTGT
CTAGAGTACGCCTCCAGGGGTAAGGTGTCACTTGCTTTGCAGGAGACACATACTGACAATTTTCGATTGTCAAAATAGTCACTTGAGGTCATAA
GGACTATGACAAATTGTTCCT
MOUSE SEQUENCE-mRNA
CACTAGTGACCGGAGCGCGAAGATTCGGGGGTGGGAAGGTGTGAGCCGCAAACCCAGAGGAGCCGGGAAAGAAGAGGAGGCCCGGGGGTGGTCC
GAGGACAGGCGTCCCGGCTGCGGAGGCTGCTCAGCCTCCTCACTGGTTGGCTGACTGACGGGCTAGCTGGCTGACTGCCTGCCTGACCGACTGC
CTGGCTGACTGACTCACTGACTGACTGACTCGTGCCTTGCAGCCTCGGGCCGGACGCTGGAAGCTGCAGCGAGCGGCGCGCGGAGCCGAGCCAT
GGCCTCGAGTGGATATAATCCATACGTAGAAATAATTGAACAGCCAAGGCAGAGGGGAATGCGGTTTAGATACAAGTGTGAAGGGCGATCAGCA
GGTAGCATCCCAGGGGAGCGCAGCACAGACAACAACCGGACATACCCGTCTGTCCAGATTATGAACTACTATGGAAAAGGAAAAATAAGAATTA
CATTAGTAACAAAGAATGATCCATATAAGCCTCATCCTCATGATTTAGTTGGAAAAGATTGCAGAGATGGATACTATGAAGCAGAATTTGGACC
AGAACGCAGACCTTTGTTTTTTCAAAATTTGGGTATTCGGTGTGTAAAGAAAAAGGAAGTGAAAGGAGCTATTATTTTAAGGATTAGTGCAGGA
ATCAATCCTTTCAATGTGGGTGAACAGCAGCTGCTGGACATTGAAGACTGCGACCTCAATGTGGTGAGGTGTGTTTTCATGTTTTTCCTTCCTG
ATGAAGATGGTAACTTCACAACTGCTCTGCCTCCCATTGTTTCTAACCCAATTTATGATAACCGTGCCCCAAATACTGCAGAATTAAGGATATG
CCGTGTGAACAAGAACTGTGGAAGTGTCAGGGGAGGAGATGAAATATTTCTGCTTTGTGACAAAGTTCAGAAAGATGATATAGAAGTTCGTTTT
GTGTTGAATGACTGGGAAGCCAGAGGTGTTTTTTCACAAGCTGATGTACACCGCCAAGTAGCCATTGTTTTCAAGACTCCTCCGTATTGCAAAG
CTATACTGGAGCCTGTGACAGTGAAAATGCAGCTGCGGAGGCCTTCTGACCAGGAAGTTAGTGAGTCTATGGATTTCAGATACCTGCCAGATGA
AAAAGATGCATATGGCAATAAATCAAAGAAACAAAAAACAACTCTGATTTTTCAGAAACTGCTGCAAGATTGTGGTCACTTTACTGAGAAACCA
AGAACTGCCCCTCTGGGATCAACTGGAGAAGGAAGATTCATCAAAAAAGAATCAAACTTATTTTCTCATGGTACAGTTTTACCAGAAATGCCCA
GGTCTTCAGGAGTTCCAGGGCAAGCTGAACCTTACTATTCTTCATGCGGGTCCATCTCGAGTGGATTGCCACATCACCCACCAGCCATTCCCTC
AGTGGCCCATCAGCCTACAAGCTGGCCACCAGTGACTCACCCCACCTCACACCCAGTCAGTACAAATACCCTGAGTACTTTCTCAGCGGGAACA
CTCTCCTCTAATTCACAAGGTATCCTTCCATTCCTAGAAGGGCCTGGTGTCAGCGACTTGAGTGCATCTAATTCGTGCCTTTATAATCCTGATG
ACCTAGCTAGAATGGAAACGCCATCCATGTCACCAACTGACTTATACAGTATCTCTGATGTCAACATGCTATCTACTCGGCCTCTGAGTGTGAT
GGCACCCAGCACTGATGGCATGGGGGACACTGACAACCCAAGACTTGTGAGCATCAATCTTGAAAACCCCTCATGTAATGCCAGGCTAGGCCCA
AGAGACCTGAGACAACTACATCAGATGTCTCCTGCCAGTCTGTCCGCGGGCACCAGCTCCAGTTCTGTTTTTGTTTCACAGTCAGATGCATTTG
ATAGATCAAACTTCAGTTGTGTAGATAATGGCCTGATGAATGAGCCTGGACTATCAGATGACGCAAATAATCCTACCTTTGTTCAGAGCAGTCA
CTATTCAGTTAACACTCTGCAAAGTGAGCAACTGAGTGATCCCTTCACATATGGTTTTTTTAAAATATAAGTTGTAGGACTTAAATAAGTATAT
AGAATATTAAGAGAAAGTTCAGAAGTCAAAACTTTAAAAGCCGCTACTGTTTCTTACTTTGAAAGATATGCCTAAAGTCCACGTGCTTGCTGTG
AGTTGTTTTCTGTATTTCCCCAAGACAAACTATGTATCTTTGCTAATGTATTAGTTAAATGAGATGATATTTGCTTTCAAGCAGATTTAAGGTG
AAAAAGTGTAATGGCTATGCATTGGGTAAATAAACTCATATTATTGTTGAGGCCCATAGGCAAAAAAAAAAAAAAAAAAAAAAAAAAAAAAAAA
AAAAAAAAAA
MOUSE SEQUENCE - CODING
ATGGCCTCGAGTGGATATAATCCATACGTAGAAATAATTGAACAGCCAAGGCAGAGGGGAATGCGGTTTAGATACAAGTGTGAAGGGCGATCAG
CAGGTAGCATCCCAGGGGAGCGCAGCACAGACAACAACCGGACATACCCGTCTGTCCAGATTATGAACTACTATGGAAAAGGAAAAATAAGAAT
TACATTAGTAACAAAGAATGATCCATATAAGCCTCATCCTCATGATTTAGTTGGAAAAGATTGCAGAGATGGATACTATGAAGCAGAATTTGGA
CCAGAACGCAGACCTTTGTTTTTTCAAAATTTGGGTATTCGGTGTGTAAAGAAAAAGGAAGTGAAAGGAGCTATTATTTTAAGGATTAGTGCAG
GAATCAATCCTTTCAATGTGGGTGAACAGCAGCTGCTGGACATTGAAGACTGCGACCTCAATGTGGTGAGGTGTGTTTTCATGTTTTTCCTTCC
TGATGAAGATGGTAACTTCACAACTGCTCTGCCTCCCATTGTTTCTAACCCAATTTATGATAACCGTGCCCCAAATACTGCAGAATTAAGGATA
TGCCGTGTGAACAAGAACTGTGGAAGTGTCAGGGGAGGAGATGAAATATTTCTGCTTTGTGACAAAGTTCAGAAAGATGATATAGAAGTTCGTT
TTGTGTTGAATGACTGGGAAGCCAGAGGTGTTTTTTCACAAGCTGATGTACACCGCCAAGTAGCCATTGTTTTCAAGACTCCTCCGTATTGCAA
AGCTATACTGGAGCCTGTGACAGTGAAAATGCAGCTGCGGAGGCCTTCTGACCAGGAAGTTAGTGAGTCTATGGATTTCAGATACCTGCCAGAT
GAAAAAGATGCATATGGCAATAAATCAAAGAAACAAAAAACAACTCTGATTTTTCAGAAACTGCTGCAAGATTGTGGTCACTTTACTGAGAAAC
CAAGAACTGCCCCTCTGGGATCAACTGGAGAAGGAAGATTCATCAAAAAAGAATCAAACTTATTTTCTCATGGTACAGTTTTACCAGAAATGCC
CAGGTCTTCAGGAGTTCCAGGGCAAGCTGAACCTTACTATTCTTCATGCGGGTCCATCTCGAGTGGATTGCCACATCACCCACCAGCCATTCCC
TCAGTGGCCCATCAGCCTACAAGCTGGCCACCAGTGACTCACCCCACCTCACACCCAGTCAGTACAAATACCCTGAGTACTTTCTCAGCGGGAA
CACTCTCCTCTAATTCACAAGGTATCCTTCCATTCCTAGAAGGGCCTGGTGTCAGCGACTTGAGTGCATCTAATTCGTGCCTTTATAATCCTGA
TGACCTAGCTAGAATGGAAACGCCATCCATGTCACCAACTGACTTATACAGTATCTCTGATGTCAACATGCTATCTACTCGGCCTCTGAGTGTG
ATGGCACCCAGCACTGATGGCATGGGGGACACTGACAACCCAAGACTTGTGAGCATCAATCTTGAAAACCCCTCATGTAATGCCAGGCTAGGCC
CAAGAGACCTGAGACAACTACATCAGATGTCTCCTGCCAGTCTGTCCGCGGGCACCAGCTCCAGTTCTGTTTTTGTTTCACAGTCAGATGCATT
TGATAGATCAAACTTCAGTTGTGTAGATAATGGCCTGATGAATGAGCCTGGACTATCAGATGACGCAAATAATCCTACCTTTGTTCAGAGCAGT
CACTATTCAGTTAACACTCTGCAAAGTGAGCAACTGAGTGATCCCTTCACATATGGTTTTTTTAAAATATAA
HUMAN SEQUENCE - GENOMIC
AAACCAAAGCCTGCATATGAAAAAGCTCTAGAAGACAACACATACACACATACTCACACATGCACGTAGGTGATGTCTGTATTAGTGAGGCAGA
ATTATAAGTGAGTTTTCTTCTTTTCTCCAATCTTTATTTAATTTTGTTTATTTGTTTTGTTTTATAGAGACAGGGTCTCACCATGTTGCCCAGG
CTGGTCTTGAACTCCTAGCCTCAACTGATCCTGCCACCATGGTCTCCCAAACTGCTGGGATTACAGGTGTGAGCCACCATGCCCAGCCTTTCTT
TAATGTTTTATATGTGTCTTTTTTTAGGTTGTAATTTAAAAATAAACATATTTTAGCAAAAAATTAAAGACTTTGAATGTTATTAAAGAATTTA
GGTCCGGGAGTAGTGGTTCACGCCTATAATCCCAGCACTTTGGCAGGCTGAGGTGGGAGGGCTGCTTGAGCCCAGGAGTTTGAGACCAGCTCTG
GGCAACAATAGTGAGATTCCATCTCAATCTAAAAATAAATAAGTAAAATATACATATGAACTTAGTAACTGGTTGGCCCAGCTTGCTTGCTTTC
ATGACAGTAATCCATGATTTTGATACCCGTCTTAATATAATCACTATCATACCTCTCTGCAGGCATAGTTATCACTTTGGGTAGGAGTTGAGCC
TTGTGCCTCTTTTTGTTTTTCTTGATGGTATCTAACGCCCAGTGGTGATACATTGTCACTCATATCAATACTGGTGTGATTCAATGTGTAATAA
TTTATATTTTATTCGACATTTAATGAGCATCTACAATGCTAAAGGCACTGTACTAGCCACTCTAGGGAATAAAAGATGAACAAGACACAGTCAA
TGCCTTTAATTAAAGAAGTAAATGCACTGATTTTTACTTTTCCTACACAGGTATTGAATTGAGAATTTTGGGGAAAAGAACATGAAGAGTCTTA
AAAGATATGGAACCTGTGAACGCTAAACCTTCATATATTTGCATTTTTATTAGTAAGATTATGGCTGCCAGTTATATTGTTTTATATCTTAGGT
CATATTTTGAAATGTGATGTGACATAGTGAAAAAGAAACAAAGAGTCTGGAGGTCTGGTTTCAAGTTTCAACTCAGCAATTAACTACCTAATGA
ACACGGGCAAGGTACTTAAACTTCCTGTGCCTCATTTTCCTCATGTGTAAAACGAGGGAGATGAACTAAATGATCTACAGGGTTCCTTCCAACC
TTAAAATTGAACTAACTCTCTCCTGTTGAGACATAAAAGAGGCACCATATGACTTGAAGGTGATGATGACTTCATTACTTGTTCTGAAAAATGT
TAATTCTAAATTGGAACAAGTAATCACTGGATCTCATGGTTGTGACACATCAGACATTACTACAGTTAGAGAAACCCAAGTTAGCACACTGTGT
CATACTTCTAGGCAGTGCTATATACGCAAAACTAGCATACCACAGTATAGGGCCTACACAATATAAACAAAAAGATTAAAAAGAGATTTTTTTT
TTTTTTGAGATGTAGTCTTGCTTTGTCGCCCAGGCTGGAGTGCGGTGGCACGAGATCGGCTCACTGCAACCTCTACCTCCCAGGTTCAAGCGAT
TCTCCTGCCTCAGCCTCCTGAGTAGCTGGGATTACAGGCGTGCACCACCATGCCCAGCTAATTTTTTGTATTTTTAGTAGAGATGGGTTTTCAC
CATGATGGCCAAGCTGGTTTCAAACTCCTGACCTCAAGTGATATGCCCGCCTCGGCCTCCCAAAGTGCTAGGATTACAGGCGTGAGCCACTGCG
CCCTGCCATTTTTTTTTTTTTTTTTTAAATAGAGACAGGGTCTCGCTATGTTGCCCAGGCTGGTTTTGAAATCCTGGGCTCAAGCAATCCTCCT
GCCTCAGCCTCCCAAAATGTTGAGATTACAGGTGTGAGCCGCTGTGCCCAGCCAAGAAAATTTTTAACAATGGATATTTTACGAATGCTTTTTG
GAGTTTTTATGTACTAAGATTTCATTTCTCTGGGTGTTCATTAATCTTATGTTTCAGTTCCTTAAATGAAAATAGGTGAGTGGGGCAGGTTTCC
TCAGAGGTATAGTTTTTACTTTAGGGACTATCCCTTTCTCCTAAAGTGTGCTTTAAAGACGGCAGCACATGAACTTGTCTTAGATAGGAGGGAC
AGCAGGCAAAAGTGATGACAGGATCTGGGTCTGCTGCCACACACCAAACTATTTATGAATTTTTTTTAAGAGACAGGGTCTTGCTCTGTCACCC
AGACTGGAGTGCAGTGGTGTGTTCATAGCTCACTGCAGCCTCAAACTCCTGGGTTCAAGTGATCCTCCCACCTCAGTCTCTCAAGTAGCTAATA
TTACAGGTGCATGCCCCCAAACCTGGCTAATTTTTTTTTGTTTTTTTTTGTACAGATGGGGGTCTCACTATGCTGCCCAGGTTGGTCTCCAACT
CCTGGTCTCCAAAGTGCTGGAATTACATGGGTGAGCCACCACACCTGGCTCTCATTCTTTATGGGTTTTTTGTTTTTTTTTTTTTTGCCCTTAA
GTTTTGTTTTTTACAACCTTATAAATCACATAATTGAGTTTCAGAGATCTCAAACAATACTAATGCAGTAAGTAAACAGGAGAGCAGGAGTGGG
GTTTGGCCAACAGTACTGGTAAAATTCACTCATAGTGGTTGAACCCAGCTGTTGCAGCTATTTGTCCTTAATAGGAAGGACACCTCCTCACTAC
AGAGAGTTGGGCAGTGATTTGCAGTATGCTAACTGGGACTAGGGCATACTCTCGATAAGGTTTAATTTTTTGTAAGAGAACCATGTGGCTTAAA
TTGATGTAAAGCATCTACATAAACTACATAACGGAAGACAGGTAACTCAGATAATGGGGGTATCAAAGACGAAATTTGGCCATTTATCTAGAAT
TATCTGAGGGAGAACCCCAATGAATCCAACAGATACAATCACTCTGGTTATTAGAGTAGTGTAAGAATCACAGTCATACGTGCTGACTTATTCA
GAGGGCAAAGTACCAGTGTCTGCAACTTATTTTGAAATACATTTTTAAAAACCAGATAGATGAATGGATAGACAGAGGGATGGAAAAATAAACA
GACAGGTGATAAGGCAAGTGCAGCAAAATGTTAATGGTGGAATCTAGTTAGTGGGTATACATATGTTCCCGGAAAAAAAATCTTTCAGTTTGGC
TGTATATTTGAATTTTTTTTTTTTTTTTTTTTGAGACGGGTCTTTCTCTGTTGCCCAGGCTGGAATGCAGTGCCAGGATCATGGCTCACTATAG
CCTTGACCTTCTGGGCTTAGCTGGAACCACAGGTATGTGTCACCACACCTGGCTAATTAAAAAAAAAAAAATCTTTTGTAGAGATGGTGTCTCA
TTTTGTTGTCCAGGCTGGTCTTGAACTCCTGGATCAAGCGATCCTCCCACCTTGGCATCCCAAAGTGCTAGGATTACAGGCATGAGCCACCATG
CCTGGCCCCAAACATTTTTATGATAAAACAGGAAAAAAACCTGCCAACATTTCTGAGGGCTTTCCCTGTGTCAGGCACTTTGTATTAGTGATCT
CAATTAGCCCTCACAAGAAGCCTGTGAGGTAGGTACCATATTACTACCCCTATTTTACAGATAAGCAAACAGGCACAGAGAGGCTAAGGGACTT
GCCCAGAGTCTCACAGCTAACAATCTAGGACAAGCTAAGATTCAAATCCAGGCCTGTGTGTTTGCAAAGCCAGGGCCTTTAGCTGTGAAACACA
GAGAATTCCGCAGATGTTTTTCAGAGCCTCCATTAAATATTACTGAGTATTATAAAACTTCTAGAACATAGGAAAATATTTTCATAACCTCAGT
CCAAGAAGAGTTCTCTAACATTCACAAAAAACACTATCAAAGAAAATAAATCAGACTTAATTAACACTAAGTTAAAAAGCTTCACTAAGATAAT
GAAAAGGCAAGCCACAGACTAGGAGAGGATACTTGCAATGCATATATTTTATAACAATTCTGTGGCATCCATAGAGGTGCTCTGCTCAGATCTC
CATTCTAGAGAGGATCTGCCTCAGCTTTCAAGCCAAAGCCACATATTTCCTTGGAGCCACCTACCCAATTATTAAGTACAGAGGTAGTATAAGG
GCTAACTATCTCTGTGCACAGTGTTTGTGTTAGAGCTCCCCATTGGATTTACTGAGACTTCCTCAAAGATGCACTCCCATCTCATTTTCTTTCT
ACCAAATTTACCTTCCTTCCCCCTTTCTTTTCACCAGTGTCAGACCTGCATCTTAAGATTTTCCCTGCCTATAACTTTTTTCTTTCCCCTTTAT
CTTTCATAGATGTTACCCCCAATAAATCTCTTGCACTGTTAATTTCATATTGGTACTTGCTTCCCAAAGGACCCAACTGGTATACTCATATCCA
GAATACATAAAGAATGTCTACAAATCAATAAGAAAAAGATAGATAATCCAATTTTTTTTTTTTTTTGAGATGTTTTCTCGCTCTGTCCCCCGGC
TGGAGTCTAGTAGTGGTGACATGGCTCACTGCAAATCTCTCGTCCCCAGGTTCAAGCAATTCTCCTGCCTGAGCCTCCCCCGTAGCTGGGATTA
CAGGCACCTGCCACTGCACCCAGCTAATTTTTTATAGTTTTTAATAGAGACGGGGTTTCACCATCTTGGCCAGGCTGGTCTTGAACTCCTGACC
TCATGATCCACCCATAATTTTGTTATGTAAGCCCTATCATTATTATTATTATTATCATTATTATTATTATTATTAAGACAGAGTCTTGCTCTGT
TGCCCAGGCTGGAGTGCAGTGGTACAATCTCGGCTCACTACAACCCCTGTCACCCAGGTTCGAGTGATTCTTGTGCTTCAGCCACCTGAGTAGC
TGGGATTACAGGCATTCGCCACCACACCTGGCTAATTTTTGTATTTTTAGTAGAGACAGGGTTTCGCCATGCTGGACAGGCTGGTCTCAAACTC
TTGGATTCAAGTGACCCACCTGCCTCAGCCTCCCAAAGTGCTGGGATTATAGGAGTGAGCCACCNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNGAGTCTCGCTCTGTCGCCCAGGCTGGAGTGCAAGTGGCATGATCTCG
GCTCACTGCAACCTCCGCCTCCCAGGTTCAAGCAATTCTCTACCTCAGCCTCCTGAGTAGCTGGGGTTACAGGCGCCCGCCACCACGCCTGGCT
AATTTTTGTATTTTTAGTAGAGACGGGGTTTCACCATCTTGGCCAGACTGGTCTTGAACTCCCAACCTCGTGATCCACTTGCCTCAGCCTCCCA
AAGTGCTGGGATTACAGGCATGAGCCACTGCGCCTGGCCGGCTCTTTCTTAAACTGAGGACTCAGCTCAAATGTCACCTCCTCAGAACTTTCCT
CTCCCCCAGTCACTTTTCATTTTGAAATTCCTTCAGAGAACTGAAAATGTTTTCCGTCTCTTTGAAATGTATGTACATATTTTAAAAAGCTAAA
TAAGCCTCTTGCTAGTTTTGCGTCCTCAGAAGGTCTTTCTTGCGGGCCTCGGAGCCATCTCTTGGAAATGTGAACATCAAGGATGGTGCTCCAT
GTTTCTGTCTCTAGGAGGGTTTAAGCCTTGGCTCTTGGTTCCAAGATAACTTCCCATTATTTTCCTTTGGATAAAGACAATTAATAGGTATGTA
ATGGATTGTACGTGCCTGACCATATGAAAAGGTGAGATTTCCTTTTTTTTGGTCTTTGCACTCTCTTCAGTGGATTGCCTGTGATGTGCATCAT
GGTACGTTTTAGTACATATCCAATAATAAAACTGTTTCATTCTTTTGTTGTACATTGCGCATTTCGTGGAAAGAACAGTTTGGGGTTGGCAGGA
TATTTTAAATTATTTGCCCATATGGGTCCCCCAGTTTTGGGCACTTGTATGGCAGGCTGATATTCCCCATTACCTCTCCGTACTAATTTTAACT
AGTAAGGGACAGGGGCAAGGGCATGGAGCTTTGGAGTCAGACAACCCTGGCTTTAAACTGTGCCTTTTATCAGCATTCTGAAGTACTGTATGTT
GAAGTAACTTCTCTGAACCCCGAATACTTCATCTATAAAGTTAGGAATAATAAATACGTCATAGATTAAGTATGGACATTATCTGATGCATAGC
AGGTACTATTCAAATGAAGGAAACGAAGTTATCCTAGGTCCTTTAATGCACTAAATGTGTGCTCCATTTCCGCCTTTCTTGGCATCTGTGTGAA
ATCTCAGGATATAGTGTCCACTAAACTTCAGTGCACTGCCTAATGAGACAACGAACTCCAAGTGCGGACCTTTTGAGTTTAAACGGGTTTCAGT
AGGTAAAATACGTAAAGTTCACAGTACCCTCGCAATTTAGATGGAGGAAAAAAAATCAGTCGGGGGGGGGGTCCCCGTATGCAAATACAGGAAT
TACAGAAGAAGACAGGACCCCCGCCGCAGCACCACTTCCCCCTACAGGGCGCAACGTCCCGCCGCTGGCGCGGGGGTGGAGATTTGCATGTGCG
AAGTCGGGAAGAGGGGGAACCACCTCTCGAAAACCCCGGCCGTCGCCCTCCCAGGCCGGGAGAGTCGGCCCCGCGCGCCCCCAAAAGCTCCCCA
GCGGCCGGCCCTCTCTACGTCATCCGCACCCTCCCGCCCGCTCCTGAACTTTAAACCACACTCGGAAGAACAACCTGGAGGAGGGGGATTTCTC
AGGAGCCAGCGGCCTTCTCACGCTGCTAGCCGTCACCTCCCGGCTCCCGCCGTTCCCTGCGCTCCCAAAGCCCACCCCAAGCTCCCGCTCCCTC
GACCCCCTCCCAAGCCAGCCGCCGCAGCAGCGCCGGGGGCGTATGCGTGGGGGCCGGCGGGGAGCGTGCGCGGGGACAGGCAGGGGCGGGGCGC
TGGGGGCGGTCGCGCGCGCGGCGGCCGCGAGGTCGCCCCGCCCACGCCTCCGGGTGAGGGTTGCTGCGTCGGGCCTACGTCAGCCGCGGGAAAT
TCCCCTCCCGCCAGGCTGCTCGCCTCTCGGCTGGGCCAGCACTCGGCTCTCCCCGCTCCGCCCCCTGCCCCTGGCTCCCGTACGGTGGACGGCG
ACGCTGGGTGACCCGGGGTGCAAGAATTCAGGGGTTGGGAAGGTGTGAGCCGCAAACCCAGCGGAGGGCGGGAAGAAGGAGGAGGCCTCTAGGG
TGGTCGGGGGACTGGGGGCCCCGCCGGCAGAGGTCCCTCGGCCTCCTGACTGACTGACTGCGGCCGCCTCCGGCCAGGACGCTGGGAGCTGCCT
GCGGAAGGTGCGGGGAGCGGAGCCATGGCCTCCGGTGAGTGTTCATGGGGCGCGGGCCTGGGCCGGGGGAAAGGAGCTCTTCTGAAGGGGGGTC
CTGCCGCAGTGAGTTTAGGGAGCTCAGTTTTTGCTGGGGCGCGTTCTGTCTTTAAAATCTCATATGGTAAAATTGTCAGCTTTTAAAAAAGTTA
CCTGCTTCAGGGTTTGCGTGCAGAATCCAAGCCACGCTCTGTAATTCGATCCTGTTGTCATAGCAGGAACCTTAGAAATTCCCAACGACTTGAT
TTTAGGATTAGGGAAGTCATAAGCGAACCAAAGGAGGACGAAGAGTTCTCATTAAAAATGTATATGTATTGAAGATGGTGGTTTTATGTTTTAG
AACAGGAATGAGGAACCTTCAGACCTCGGGCAAGCTCTCTCCCCCTTTTTAGTTTAATTTCATCAAATGCAAAGAGGTGATCCCCCTTGTACTG
ACAGTTGTACAAAATGTGTCACGGCTTTAAAAATCATCCGTGGTATTTTAACACGAATTAGAAAATCCGTGGTATTTTAAGTGTTAATGTTGCT
TCGACCAAGTGTAGGCCGGGCGCGGTGGCTCATGCCTGTATTCCCAGCACTTTGGGAGGCCGAGGCGGGTGGATAACCAGAGGTCAGGAGTTTG
AGACCAGCCTGGCAAACATGGTGAAACCCCGTCTCTACTAAAAATACAAAAATCAGCGGGGCGTGGTGGCGCGCGCCTGTAATCCCAGCTACTC
GGGAAGCTGAGGCAGGAGAATCGCTTGAACCTGGGAGGTGGAGGTTGCAGTGAGCCGAGATCGCGCCCCTGCACTCCAGCCTGGGCGACAGAGG
GAGACCCTGTCTCTATTTAAAAAAAAAAAAAATGTAGTTTTTACTGTGAGATAAAGCTCAAGGTTCCTAATCTGACGAATAGAGACGTTGAATC
CCCCTTCCTCCTCGCTGGAAATAAGGGGAAAGAGGAGTTTTAGAATATCCGAGCTTTAAAATAATGAGTAGGAATGAATACAACTTTATCACTT
ATGTGACTATATATTTTCCCTTAATAAATGCTCGATCTCTCCCTTTGGGGGGGAATAAGTTTGTAAGAAACATTGCAGAGCAAGAAAGTCTCCT
ATATACACAGATGTGGTTGGGGCTGCCTGGAGCTTAAGGTTGAGGGGGACGGGAGAGCCGGGGCGGGGGGATAAATCAAGTAGCCTGGGGTCGA
GTTGGTCTTTTACCAGGATTTTGGCAAGGTGAGTGGGGGACACTTTCCAGACAGAGGAAGCAGCGTGAGCAAAGGCAGGGAGGGCTAAATGCAT
GTTAAATGCTGTACTAAGAGAATGAAGAGGGGGCCCCGTTTATGAACTTGACTCTTATCCCCAAACTGCCTGGTCCAGTGACCCTCAGAAGTAG
GCTGATGTCTAGTAGAGATGAGGAAGTAGGAAGCTTAAAGAACCTTTTTAAAAGGTTGAGCAGAACAATGCCATAGCTATTGCTGTAGGCAGAA
AAGGAAATTGGGGTGTTTCACATGACATTTGATTTCAGTTTGTTTGTGGTTGTGAGAATACTAAGTGTAGTTTAAAATATTTCAACTTTCCCTT
TTCCCACTTAAGTGACACCAGACTTTCAGATAATCGTGTAAATTTATGAATTCAAAGCATGTGTAGATTAGAAGTGCTAAGATGACTTCAAATA
TTTTGTTAATGTATTGGTTATCAGAAAGATTTTGTTTGGAAGTAATGGAGAATGCCTAGCATTATAAGTAAGTTCAGGCTCTTGGCGATTTAAT
CAAACTTTATTGATATATTGCATGAATTTAATACTTCTAATTTTTTCAGTTGAGAGAGGAAACAACCATTTCTATATAGAGTATTGTAAGACCC
ACAGGATTTTAAAATTTGATACAATTTAAATTTGTTCATTGACACTGTATTTTGTGCTGTTGTGAGGAGGAAGGATTCCCTAAAAGTAAGGAGG
AAATGTTTAAAGAAATGGAAAGTTTTAAAGGTTTAGAAGTTGAAGGGTGATTAACTTTTTTTTTTTTTTTTTTGGTCTTTTCTAAGAGTTGCAT
TAAAATGGGACTATTTAAAATGTAACAAGTTATTTAAATTGTAATTCAGTTGTTAGAAAACTGCCAGGCCTGGAATAAAAAAAAAAAAATGTTT
ACAGGAATCTGTGTACTTTTCCTTAATCTGTTATAAAGACCTTAGAGAAAGAAAAAGATTCACTAATAAAATTCTAAATTCCTATTCTGTTTTG
AAACAGTAATTTAACCGGAAGAGTGACCAAAAAAAAAAAAAAAAAGAAATTGCTTAGTTTTTCATTGTCTAGTTTAGATTCTGTTTGTAGTAAG
GTAGTATTAAAACCTCAGAAAGTAACTTTTATTCATTACGAAGTATTTAAGAGCATTTCTTCATTTCTTACCAGATGTCATATGTAAATAGGTA
TATGTAGTTGAATATGTGATAGCTATAATGTAAAAGTAACATAAATCATAATCTTATAAGCAAAATTATGTAAGTTAGAATGTAGTTTAAAATC
ATGTATATAATATGATTCTAAATTTTGTTTAGATAAGTTAGCAGATTAATTAGGATTTCACAGCTTGTATTAAGCTGCCTTTCGTTGCTTTTTA
AAAAAATTACACCTTGATCACAGTCTTTTGACATAATTAATAATAGATATAGTGAAGAAAAATGTTCCCAAACCCATCTAAGGTATGCATAGTA
GGCAGTTGTTGCTTTTGTATATTTATTCTATTTAATATAAGTAAAATTAGATATTTTATGTCTGAATTTAAACCTTCTCATTGTTCCAGTAAAA
AGAGCAAAATGTCTTTGTGTGGCATATAGCTCTGTTAACTAATAATTAGTGCTGTCTTTGGTAAAATGTTTTAAAATTTAGATAATTTATTTTT
CTTCACATTTTTCTTAGTATGGTTCTTCTGTGTCACACAATAGGAAGGAAGTACAGAAATGAATTGTTTTTGTAAACCTCACTTCTTACCAACT
ATTTGTAGTGTGAATTTCAAGAGAATTGTTGTTTTTCTCGATTAAAGGGGAGAAATGGAAGTAAGTGTGAGGCAACTTAATCTTTAATAACAGA
GCTGGAAGTTAGGAATGGATAGCTACAGTAAATGACTTCTTTTTTTAAGTAAAATGCCACACATGAGCATGAATGTTTCAGAAATGCAACACAT
GAAAAAATAGAAATAACAATGGAGTTAAATAATTAAGGTGACGTATGATAGGCACAGATAAAGGGAATGAGTTAATGTAATGACTAGGGAGGAG
CCAGACAATGAATCATAATTAGGAACATCTAGGTTATGCATAGTATCTTTCTCAAATACAATTTAAGGGACTCAGAATTTCATTTTGTTCCAAC
TTTCTCTCATATGGGTTCTCTGAAAATGATTTTGTTTGTAGGCACATGACCCAAATATTGCATGGGATAAAATTGTTTTTCTGAAATTCATATT
TATCTGGGCCTCCTCTGTTTTTATTTTTAAAACAGAGGTGATTTGGTCTTGCCATTTGTTTTTAACTTTAATACCTGTATAATCTAGGACAGCT
TTTAAAATTTATTTATTTTACCCCTGGCACGTGCAAGAGTTTTTTCAAGACTTACTTTTTTCCCCCTTGGTAACAGAATCCCTATTGCCAGAAT
CTTTTACAGTTTTTACCTTGTAGTGCATAAATCAGTTGAATACTCATCTGAAAATTTCCTTTCTTTGATTCTTAAGGCCCAGTTGAAAGTCAGC
CTGATGTATTGGAGTAATAAAGTCAGCCTGATATATTGGCATAGCCTTTGGCTGATGAGAGACCTAGTTTGAGGAAAAAAATAATAGATCTTAT
TTTCTTTTTCTCCAAATTGAGTAGAGGTGATATTTGGAAAAATATATATTGTTAGCTCCATTGGCTCCTTTAGGTCTCAGGCAAAGTCTGTCAC
CGAGGACCTTTATGAGAGACATGCAGTCTAGTCAGCTATTCTCAACTGTGGTGTTGACACACTGGTGTGACAATCATTTGTGTGATGTGTTGCA
AGAGTTAAAATATTTGTGTTGTAAATTATTTACTTTTTAAATAAATGAAAGCATTTGAAGGTTATCTCTATATTGTCATTTTTTCTTGCTTGCA
TTTGAATAAGGTTCTTTTTTAAGTGGGAGATAAACACAAGTTCAAGTTACAAGTAGCCAGAATTGCTTTGCCTAATTTACTCACGTCCCAGTCC
ATATTTGTACCTGAATTTTTAGATTACTTCTCTCAATATGTGCATTGTTGCTTCCACTTCATGTTTTAAGACAATTATTCTTAATCAGGAGTGT
GTTAGAATTTTCTTGGAAGGCTTTTTCAGCAACGTGTATCTGGGCTTTAAAAACTATGAAGATTTCTTTATCTTCTAAACATCTCAAGTGGGTA
AGGACATGTATGTTTTTGAAAAAACTACCTAGGTGATTCTGATGTACATCCCCAGTATGAACTCTGTTCTAGGGATTTCTTTGGATCTAATACT
CATTTGGCTATACCAGGATGCATTATCTTCTTAAATTAAGTTGCATTTATTTTAGATTGCAGATTATGTGCTGTATCAGTTATTACCTCCAGTT
GCTTTTATGCTTGTTTTTTAAATTTGGCATGCCATATTAGCATACCATTCATAATTCATTTTTTATTATAACTTCATATAATTTTCAACTTTAG
TTTTATCATGTGTAAGATGGGATAATAATTTCTATTATTTTTCAAATGGAGATTAAAGTTAAGAGGATCAAATGAGAAATGTATATAAAGGGGT
CTTAAAATAATGTTATAATTACAATAATTAGCTTTTTTTTCTGTATACTTGATGCTGGTTGCTAATTCTAGCAATAAAGTGTTTGTTAATAATG
TGTCTATTTCCGATAATTTATGATCCATTTAAACATTTCAATGTAATTTTTTTATTTTAAAAGCTCATGCTTTATTAGATAGGCACTATGTTTT
AAAATAGCACTCTTTCTTGCCTAAGCGCAAATAAAAACATAGATATATCTTACAAACATTTTGGCTGTATTTTAGAAGTTATTCTGATGTATAT
TAGCCTTTTAAAATACTATTCAGAATATAGGGGTCAGTCCCAGCTTTGCATAGTGTTGCTTAAAGAAAAATACAGTAATCAATTTGTTTAGAGT
CAATTTTGGAGACGTTTTGGTGCTTTTCAAAACTGTGGCGATTTGAGGTTATCTTAGTTAAGAGTACTTATTTTCCCTACAATTTCCTGATTTA
TGTTTTACCCTCTTGCACTCCATAGATGACTACATTTCAACACATTTTATATTCAGGAAGAATGGCATTGTGCAAAGCACAGTACTGAGGAAGG
AGATAACTGGTGCATTGTACTGATGCATTATATAAATGATTTATAGCTGGGAAGTTTTTTAAAAATCATAAACACAGATAAATCAAAATAGGAA
TGAGTATGAGGAAATACTGAGGTGTAGCAATCCTGGTACCTATAAAGCACTATTTTTTTTTCCTTAGTAGTGGAATCTAATTAAAAAATCTGAC
TAATCACTTATATGATTATTTAACGTTTGAATAACTTGTATAAACAAAAGACCTTAAGATTTTTTAGTCATTCATTCAATAGATGTTTAATGAA
TATCTCTGTACAAGGCACTGATGAGTTAAACACAATTTCTGCCTTCAAGTAGCTTGAAGAAATAAAACAAGTAGCTTTATCTCAAGAAGAAATA
AGACGGTAAGTTTTCCCTAAATTACAGTTATTTGGAATAATTGCTATACACAGAATACTATGGTAGTTTAAAGGAGGGGAGATTATTTCCAGCT
GGGGGAATGTAGAGTATTACAGTATACTGTGTTACTTGGTTGTAATACTCTACATTCCCCCAGCTTGTTAATTTTTACCAATTAAGAGCTTCTG
CAGTAGGAAAAATAAATTCCAAGGACTTTAAATACTCAATATTATATGATAACTGATATTTATGTAGTTTTTACTATTTAAAAATATTTAATCT
TTTCAACAACTACTTTTCTGTTCTTTTTGTAAACAATCTAAAGAAGTGTCATGTAACCAATGAAGTTTTGGGTTTTTTTGTTGTTGTTTTTGTG
TTTTGTTTGTTTTTTTTTTTGAGACGGAGTCTCACTCTGTCACCCAGGCTAGAGTGCAGTGGTACAATCTTGGCTCACTGCAGCCTCCACCTCC
TGGGTTCAAGCAATTCTCTTGCCTCAGTCTCCCAAGTAGCTGGGATTACAGGTGCCTGCCACCACATCCCGCTAATTTTTTGTATTTTTAGTAG
AGATGGGGTTTCACCATGTTTTCCAGGCTGGTCTGGAACTCCTGACCTCAAGTGATCCACCCACTTTGGCCTCCCAAAGTGCTGGGATTACAGG
TATGAGCCACCATGCCCAGCCTCAGTGAAATATTTGAATGCTCACTATGAACCTAGCAGTTTTATTCACACTCTCTCTTCCCAAAGTTAAACAC
TTATCATTAACGATGTCATTTTATATCAGTTTCCCACCAAGACACCTTTAAATAAGTAGTGGGCAGGGATTTATTTGGCCTTTACTTCAGCCAC
ATAAATTATTTAAAATGTGACTTAAAAAGTAATGTTGATTTTAACAGTTTTTAAGAGGAAAATATACTTTCTTAAATACTCCTACAAGGATATA
TAAGGGTATGAAATATTTGAAATTGCAATGTGAATTTGAAATCTTTAAAATATGCAAGGATAAAATCGTGAAATACTCATCTGCATATCTTATG
TTTTTGAGTAAAACTGAAATAAGGATTATGAACTTGTTTAGGTTAAAGTCAGTTTTCTCTGGCTCTTCATCTTCTGAATAAACAAATAGGTAGT
AGATTCAAACACAGGGAGTTTACACCATACAGAATAAAATTGAAAGTTACAGTAATACTTTATTCAGTAACCCAAAGTAGAAGCCATGGAATCA
TCAATGATTCTTTAATTTCTGAGCTATCAGTGATTCTTTAATTTCTCTTAACCCTACTGTTCAGGCACCAGGTGCTGTCAATTTTATCTTTTAA
ATAATCCTTCATTCATCTTATCACTGCCATATATCAGAGATACCCTCATCATTGCCCACATTATTATAGTGGTCTCTAAACTGATGACATTGCT
TTCTTTTTTCTATCTGTTCACTGTGACTAAAACTTTTACTCTAAAATGCAAATCTGATCATGTGATTTGCCTGCATCAAAATATTCATTGATTT
CATCACACATAAAATGAAATCCACATTGCCAAGTGTGCTCTGACTTTTAGTTCTCTTTTTAAGAGAGAAAGTGTGTTCCATTTACATTGGTGAT
TCTTAAATAAACCCTCCTCTGTGGACACACTGTTCCCTTTTCCCTAGAATACCTACCTTTTTCCTCTTCATTTCCTCCGCTCTTTTGCTTCATT
AACCGCTCTTTTTCCACTGACACAGTTGGGCATTCCTGACTCTTTCGCTCTTATTTCCGCAGAATTTTGTTAGTGCTTTCCTTCCTTCTGTAGT
CTAATAATATATGTTGTTTGCCTTGCTCTTCTATAAGGTCCTTTAGAGTGACAGTTGGGATACCACCATAGTATCACTAAATCATTGTTTTCAA
GAAAGCATCTGAAATAATAGACACCATTTTTTTTTATTTTTTATTTTTTTACACTTTAAGTTTTAGGGTACATGTACACATCGTGCGGCTTTGT
TACATATGTATACATGTGCCATGTTGGTGTGCTGCACCCATTAACTTGTCATTTAACATTAGGTATATCTCCTAATGCTTTCCCTCCCCCCTTC
CCCCACCCCACAACAGGCCCTGGTGTGTGATGTTCCCCTTCCTGTGTCCATGTGTTCTCATTGATCAATTCCCACCCATGAGTGAGAACATGCG
GTGTTTGGTTTTTTTGTCCTCACGATAGTTTCCAGCTTCATCTATGTCCCTACAAAGGACATGAGCTTATCCTTTTTTATGGCTGCATAGTATT
CCATGGTGTATATGTGCCACATTTTCTTAATCCAGTCTATCATTGTTGGACATTTGGGTTGGTTCCAAGTCTTTGCTATTGTGAAGAGTGCCGC
AATAAACATACTTGTGCATGTGTCTTTATAGCAGCATGATTTATAATCTTTTGGGTATATACCCAGTAATGGGATGGCTGGGTAAAATGGTATT
TCTAGTTCTAGATCCCTGAGGAATCGCCACACTGACTTCCACAATGGTTGAACTAGCTTACAGTCCCACCAACTGTGTAAAAGTGTTCCTATTT
CTTCACATCCTCTCCAGCACCTGTTGTTTCCTGACTTTTTAATGATCGCCATTCTAACTGGTGTAAGATGGTATCTCATTGTGGTTTTGATTTG
CATTTCTCTGATGGCCAGTGATGATGAGCATTTTTTCACGTAGACAACATTTATTGAATGTGTATGTATATATGACACTTCGTAAGAATTTAGC
ACATATAATCTGTCATCTTCACAACAACCCAATGAGGTAGGTGCTATTATGATTATTCCTATTTTACAGAGGAGGAAACTAAGAGATTTTTAAT
TTTAATCACATTTCCTTGGTCTTACTGTTAAGAAGTAGCTGGGGTAGGATTCTAAAGGCCTCTTCTCCATACTGTTCTGATAAGGCCCACTGTG
TTACCATTTCTCAGGATTGCTTGCGTTCTAACAGGATAAGCCTTAGTCAGCACTAAAGTGATTAAGGGACACATTATTATTATTTTTTAATGGA
AAACATCTTCACACCCCTATTAACTCCCCTACTTGCAAGTACAGTTGAGTGTCTATTTACCTTTTCCCCCACAATTTGGATAATAAAAGTAAAG
TTGCATACATTTTAAATAGCTTTATTGACATATAATTTATGTACAACAAACTGTACTTTTAAAGTATGCAATTTGACAAGTTTTGACATATGTA
TACAACCCTGGAATCATCATCACAAGATAATGTCCCCCCTGCCTTTCCTATCCACCATATATCCCTGTTCTTAGGCAACCAGTGATCTGCTATC
GTTCATTGTATATTACTTTACATATCCTAGGGTTTCATATAAATGGAATCATACAGTATGTGCTCTTCTGGTCTAACTTCTTTCACTCAGCATA
ATCATTTTGAGAGTCGTCCATGTTATTATATGTAGCAGCAGTTCATTCTGCAGATGCATATTTTGAATTTTAGTTATTATAGAAAGTGAATACT
TTTTTTTCTCTATTATGTCTTTAGAAAAGGTTTGCATGGGACTGATTGCACATTCTTTGAGCCTAAAGTCCCTCCTGGGGCTTGTTTTGATATA
CTGTGTGTAAGTCCTTTAATATTTGAATGGAGATATAAAAAACTTACATATGCTTCTGCAAGGATTTAAATGCATTTTCATTTTCAGTGAATGG
TCTTTTCATTCAGTATTATGAATATGATGTAAAATCAAGCAGTTAAATATATTTAACTCACTTTAGGAGTGTGATATTTTACTTCTGAATACTG
CCTTCTTTACCCTTCTTGGGGAATCTTGGGGGAAACCCTTCTGCTAGGTCTCACGTGGGAGGCAGATATCTCTGAAGTGGTTATCTCAGAACTT
CATATTGACGTTAGCTATAGATATTAAGCTATAAAGTGCTAACCAGTCAATATGGTTTGAATGACTTGTTTTGACACTACATAGGGACTCAATT
TGAATGGGAAAAAAAGGTGCCTCTCCCAGCCAATCTCCAAGATAACATTTCGGTCTTGTTATTTAATGTTAGGAGGAAAAAAATCTTTGTTTTA
GTCTGCTGTTATAAAAAAAACAGAATGTTAAAATCCATTATTAATTGCTATATTAATCTCACTGACCTCCTCCTTTTTAAAAACTTTTCAGGTG
CGTATAACCGTATATAGAGATAATTGAACAACCCAGGCAGAGGGGAATGCGTTTTTAGATACAAATGTGAAGGGCGATCAGCAGGCAGCATTCC
AGGGGAGCACAGCACAGACAACAACCGAACATACCCTTCTATCCAGGTAATAGACCCTTCTTCTGTGTCTATCTCACTATTAGTTGCTTCATAT
ACTAGCTATAGCAATTATTTTTAAAGGGCCAGTTTCTTCACAGTCATCTCCACAGGTTTATCTTTTCTTTTTTCCTTTTTTTTTTTAAACGGAT
CTGTCAAAAAGACATCTATTAGATAGATGTCTAAGGACATTTCTTAAATATAAAATGGAGTCTTCCCCACTGCAGATAGTTCTATGGTGCTTGT
GTATTAACCTAAGTAGAGCGACTCAATGTTGAGAGTACACTGGCAACTTTTGGATCATGTTCTTTTTCATGGAACACAGAGATAATAAGGAAAA
CCCATTCTCTGCAGAGGTGAGTGATATTATTCTGTAGATTAAACATGGATTTGAGAACTCAATCATAATTCTTCTTGGCTAGTCTTAGGGCTAG
AACATTTTTTTCTCCTTTTTCTTAACTTTATTGCTTGTCTCTGATTCTCTGGGTCATTGACTGATTTGAAATACCACTTTATAAATACAGTTTT
TTCACATTAGCATAAATTTTAAAAAGCTTTTGACCAAAAATGACTTTGGTAAACATACTAATAGTTATAAGATGTACTAAATTTATTTATTTAT
TTATTTTTGAGACGGAGTTTCACACTTGTCGCCCAGGCTGGAGTGCAATGTCGTGATCTCAGCTCACTGCAACCTCCGCCTCCTGGGTTCAAGT
GATTCTCCTGCCTCAGCCTACCGAGTAGCTGGGATTACAGACGCCCACCACCACGCCCAGCTGATTTTTGTATTTTTTAGTAGAGACGGGGTTT
CACCATGTTGGCCAGGCTGGTCTCCAACTCTTGACATCAGGTGATCCACCCATCTCGGCCTCCCAAAGCGCTGGGATTATAGGCGTGAGCCACT
GCGTCCAGCCAAGATGTACTAAATTTATTTATTTTTTTATTTTTTATTTTTTTGAGACGGAGTCTCACTCTGTTGCCCAGGCTGGAGGGCAGTG
GCGTGATCTCAGCTCACTGCAAGCTCCACCTCCCAGGTTCAAGCAGTTCTCCCGTCTCAGCCTCCCGAGTAGCTGGGACTACAGGCGCCCCCTA
CCACGCCCAGCTAATTTTTGTATTTTTAGTAGAGACAGGGTTTCACCATGTTAGCCAGGATGGTCTCAATCTCCTGACCTCGTGATCCGCCTGC
CTTGGTCTCCCAAAGTGCTGAGATTATAGGTGTGAGCCAACATGCCCAGCCCAATGTACTAAATTTCTATTTTCTATTTGTAAACTATTTTTCT
TTGACTTTCAATGAAGGGTTTATCCTTTAAATGCCAATTAAAAACTTTAATGGCAAAGTAGATTTTGAAAAGTGTTGCTTTTTGTTTTTGTTTT
TGAAAAGTGTTACTTTTATCAACAGATCTTAATTCACTGTTTTATAACTAGATTATTTACAAAATCTATTCTGTTGATGAACTTAAAGGGAATT
GCTGAATTATAGAATAAACCAATGGCAAAATTATATATATTGAATTCCAGATTTCTATTTTTATCACATTTTTGGCAGTAATATGGATTTGGGT
TATCTAAGTCTTTTAAATGTTAAAATGGATGAGATGCTAATAAAAAGGAAATAAGGAAATACTATTTGTCCAGTTTTTTGAGTCATTTTTAAAC
TTTTCTTCATAATTCATCTTATCCTATGCATCTCTATTTCTAAGATATGTATTTTTACCATTCCAGTAGGTCCTAAACACTTTACAGCTTGAGT
TTTGTATTTGCCTACTTATAGTCTTGAGCAATTACCTTCTCTTTCATACAGAAAGAAATGTAATACTTCTTGGTTACTTTCAAATTCTATTTAA
ATTCAAAATTATGGATTTTAGTGCCCTCTACCTTGTAAGCTACCCATTCTTACTTTACCTTGCTCCTTTGTGGTATGCTGGTTTTTATAGTCCC
TGCTTTGTTCTTGCAACTTTACTAGCTTTCAAACTAAGCATTAATTCCTGTCCTCCTTCCTCTTCCATGTTAAACTCTCCTTTTTACTCTGTGT
TCTAGCTATACTTTCTATGTGAAATTTGATGTTGTTAAACTCAATTTGATTTATTGCCTATTCTAGAGTAAACACTTGGGGTATGCTTACTGAA
CAAATATTTTTGTGAAGTGAAATGTCATATATTAAGTAAAATGATCATATTTATGTGCTATCCTTTCCAATACGGCTTAAATATATTTTCAAGG
AATGTTTATGGTATGACAGTACGTTAAAGAGCTGGGCATTTAGTGACTTAACATCTGTTTTCTCATGTTCTATGGATAAAATTTGGGTAGAGTT
TTACTTGTTTTGAAAGGAAAATAAGCCCAGTCAAATGATATGACCAATTGAAATTAGGGCAGAAAATATGTCAAGAAACTGAGAGGTAAGTTTT
TCTGTACTGGTATTCTCTGGAAAGTTTCATCTAAACTGCAGTTGTTCGTCAGCTGGATTTTATTTATCTAGTGCCAATTACATTATAATTAATG
TAAATTGAATTCTCATTTAGTAGATCAGTTTATAATGCAATCTATTTGGATCATGTATTTAATTTCCCCCTTTTTTCAGATTATGAACTATTAT
GGAAAAGGAAAAGTGAGAATTACATTAGTAACAAAGAATGACCCATATAAACCTCATCCTCATGATTTAGTTGGAAAAGACTGCAGAGACGGCT
ACTATGAAGCAGAATTTGGACAAGAACGCAGACCTTTGTTGTAAGTACACAGTTACAGACATCTTCAGAAATAAGATAAGACATAGGATGTTCT
GTTTCTTCTCCATGACAATCTGTTACTTTTTAGCAGAATAATTTTCTCATTCTACTTCTATTTACTGTATTTTACACTTACATTTTTACTATTT
GCATTAGAACTACCCTTTCATTCTTTATTAGTAGACACATCTTATGATAGTTAGCAGGAAGACATTTTTATTGTTATTAAAAGTAAACGTAAAC
TTCTTGTATACTGTGATTTCCTTTCTTGGGGTGGACAGTTTTTGAAATAATTATTCACTCTGTCTTTTTTTTTTTTTTTTTTTTTTGAGATGGA
GTTTTACCCTTGTTGCCTAGGCTAAAGTGCAATGGCGCAATCTCGGCTCACTGCAACCTCCACCTCCTGGGTTCAAGCGATTCTTCTGCCTCAG
CCTCCCGAGTAGCTGGGATTACAGGCATGCACCACCACGCCTGGCTAATTTTTTTGTATTTTTAGTAGAGATGGGGTTTCTCCATGTTGGTCAG
GCTGGTCTGGAACTCCCGACCTCAGGTGATCTGCCTGCCTCGGCCTCCCAAAGTGCTGGGATTACAGGTGTGAGACACCGTGCCTAGCCTCTGT
CTTTTCATTTATAGTTAACTTATTATTATTATTATTTTGAGACAAGGTCTCGCTCTGTTACCCAGGCTGGAGTGCAGTGGCATGATCTCGGCCC
ATTGCAACCTTCACCTCCAGACTCAAGTGATCTTCAGCCTCAGCCTCCTGAGTAGCTGGGACTGCAGGCATGTACCACCATGCCCGGCTAATTT
TTGTATTTTTTGTAGAGATGGGGTTTTGCTTGTTGCCGAGGGTGGTCTTGAACTCCTGGACTCAGGCAGTCTGTCCACCTTGGCCTCCCAAAGT
GCTTGGGATTACAGGCGTGAGCTGCTGTGCCACGCCCATTTACAGTTAACATAAAAATGCCTGGATGTGAGAAAAATCAACTGGAAAACTCTAG
AGTTATTATTTAGAAAGTGGCAGGTTATAGAATTAACATACAACAATAGTTCTAAGATAAAAATAGTCATGTAATACTTCATGATAGTGAGGCT
ACCAGAAGATGGGCCCATTCATCATGTTAGAATGTAAATTAATAAAGACGTCTGGTAGGCAATTTAACTACATGGCAAAAGCCTGAAAAATATT
AATGCCTCTTGAGCCAGCTGTTCCACTTCTAGGAGTTTAGCCTCAGAAAATAATTAAGTGTGTTCAGACATTATGCTCTAATGCTGTTCATTGC
AGTTCTCTTTGTAATGGCAAAAAGCTAGAAGCAATCCAAATAGTAAGCAAAGGGGATTAATTAAATAAACTGATACATTCATGCAATGTACTCC
TGTACAGCCTTAAAAATCATATTGCCAATATGTCTATTGGTATGTAAATATAGTCACAATATATTAGATAAATAGGTTTTCTGTTTTTTGTTTT
TTTTTTTCTTTTTGAGACAGTCTTGCTTTGTTGCCCAGGCTGCAGTGAAGTGGTGTGATTTCGGCTCACTGCAACCTCCACCTCCCTGGTTCAA
GTGATTCTCCTGCCTCAGCCTCCCATGTTAGCTGATATTACAGGCATGTGCCACCATGCCCAGCTAATTTTTTGGTATTTAAACCATTAGAGAC
AGGGTTTCACCATGTTGGCTGGGCTGGTCTCGAATTCCTGACCTCAGGTGATCCACGTGCCTCAGCCTCCCAAAGTGCTGGGATTACAAGCGTG
AGCCACGGCACCTGGCCAGATAAATAGGTTTTAAAACAATATGTAAAGAATAATTTTATTTTTGTTTAAGATAAATTTATACATATACATGATG
TGTATATGTTAATACTAAGGTATTAATAGTAGTTGTCTGGGTGGGTAAGATTGTAGGGTTTTATGTTTTTCTTTTAACTTATATTTTATAATTC
ATTTTTGTAATGAACATGTATTGGCTTTTGGGGAAAAATGAACTAATGGCAACAGAAAAGAGATTAATAGTCTCCACATATATTAGTAATAACC
ATTTAGAAAAAAATACTTTAAATGCTCCTCTTACAATAATAACACAAAAGAAAATATCTAGTATTATACACACTTGGCAAATATTTGTCTATAT
TACTTGACCTCCCAACAACATTTTACACTGTGATCACTCCTTACTTGAAACATTTTTATTCCTTGCTTTCTGTGAAAACAAACTTCCATTTTAC
TTCCTGCTTTTCTGGCTGTTCTTTCTTAGTTACAGGCAGTTAAATTTTTGGACCACCTCACTTATTGTCCTAATAATGCTCTTTATGGCTGTTC
CAACTCTCTCCCCAATTCAGGATCCCATTAAGGATTACACACTATAGGTAACATTATATTTAACATGAATAACATGAATAATATTTTCATGTTA
ACATGAAACATTTTGGGCAAGAAATCTATAGAAGTGATGCAGTATAACTCTAAAAATATCCCTTCAGTTTTTTACATTATTGGCGATATTATCT
TGGCATTCTATTATAAAGAAGAACTTTCCCTTCCTCTCTTTCTCCATTTAAAAAGTATCACTGTAAACTCATGCATTCTTGTATTTAATATATT
ATAGTTATTCTGTTATTCTTTTGATTTTCAAATTGACCCAGATTTGACTAATGCAAGCCTCTTCAAACTGTCTCCTGTGTTTTTTTGATATGCC
GTCATCTTTCTTTGAGCACTTTTTTACTTTCTGACAAAATGAAATGTTCAAGACTTGTGCTTTTCTTTTTTCTTTTTTTTTTTCCTTTTTTTTT
GAGACAGAGCCTTGCTCTGTTGCCCAGGCTGGAGTGCGGTGGTGCGATCTCACCTCACTGCAACCTTCCCCTCCCAAGTTCATGCAATTTTTGT
GCCTTGGCCTCCCAAGTAGCTGGAATTACAGGCGTGCACTACCACACCCAGGTAATTTTTGTATTTTTAGTAGATACGGGGTTTCATCATGTTG
GCCAGGCTTGTCTCAAACTCGTGACCTCAGGTGATCCTCCTGCCTCAGCCTCCCGAAATGCTGAGATTACAGGCATGAGCCACCGTGCCTGGCC
AACTTGTGCTTTGCTTGCCCCAGCCCTGAAGTCATCAGCTTTTTCCAGAGATTCTTGGTTCCTTCTGGTGGGGAATGGTAGTAAGTCTGATTGC
CTTCTCAACATATTCCCTTAAATGTCTCAAAAGTACCTTAAGTAGCGGGGCACAGTGGCCCATGCCTGTAGTTACAGCTACTCAGGAGGCCAAA
GCAGGAGGATTGCTTGAGCCCAGGAGTTTGAGTCCAGCCTGGGCAACAGAGCGAGATTGTATCTCTTTAAAAAAAAAAAAAAAAAGCACTTCAA
ATAGAATATTCCCCAAACGTGATTTTACTGTGATCATATCTCTTTCCCCTCATAACTGGTCTTCTTCCAATGTGCCGTATGTTAGTGATTGCGA
TAAAAGACCTATAGGTGTCATCTCTGACATTCCTCATCCAGGTGTACACATTGAATCTACTAACAGTTACCTTCTCAACTGGTTTTGAATCTAT
CCACTTCACTCAGTGAAATTCATCACCGTCATACTACCACTATAGCCTAAACTGGGGCTGCTGGAATAACCTCCTGACTGGTCTTGCATTAACC
ATTTTGTACCTTTTCCAAACCCTCTATCTTTCCTCGCTTTAGCCAGTGTGATCTTTTAAAATACAAATGAAATTGTCTTTCCCTTGCTTAAAAC
ATTTTAATGGGTTTATTTGGCTCTTGGAATAAAGACAGCATTCCTTCTATATAGCCTATAAGGCCCACATGACCCACCTTTATTTCTAGCCTTC
TATTAAACTCTGCTTTCCTTAATTTTTATACTCCACTCACAATGCCTTCCTTTAGTTTCCTCTGTTTCCACTGCTCTTTTCTAGGGGTCTTTGT
GTGGCCTGTTCCTTTTGCCTGGAAGTGTTTTTCTTCTGTTCCAGCATAGGCTCCCATAACATCATGTATACCTCCTTCATAAAACTTGTTATGA
TTGCAGTTTAGCATTTGTGTATGTGTGATTATTTAATGTATATCCACTTGACCAGAGACTACCAGACTTTTTCTGTAAAGTACTAGATAGTAAA
TGTTTGGGGTTTTGCAAGCTGTATGGTCTCTGTTGCTCAGCTCTGCTGTTGTAGCATGAAAGCAGCCATAGATAATATGTAAACATCAGTTTGG
CTGTGTTCCAATAAAACTTTATTTGCAAAGACAGACAAGATGTTGGATTTGGTCCATGAGTTACAGTTTGCCAATCTTTCCACTAGACTGTAAG
CTTCTTGAGGAAAGGGATTGTATATCCCCATTACTTTGCAGAGTTTTTGGTATATAGTACGAACCCCAAAGCTTGTTGAATGTCTGTGAATGAA
TGAATGAATAATTGATTCAAACAATCTCTTGATCCTTCATTTCAGAATTTCAAAAATTACCACCACCCCTAATATTCCGTGAATAGCAGTCAAA
TATGAGATTATTCAGATGAAACAAAATGAAGTAATATAAATGTATTAATATGAAGAATTCTATAAAATCTTTTTACCAAAGAAAAAGAATCAAC
CAAGAATCTTTCAAAGAAGTGGAAATACTTTTGAGGAAGAGGATTATAGTTGCAAGATTTCAAGAAAGGATTAAGAATTCAAGATTTAGCTGAC
CAAGCATAGTGGCTCATGCCTATAATCCGAGCACTTTGGGAGGCTGAGGCAGGTGGACTGCTTGAGTCCAGGAGCTTAAGACCAGCCTGGGCAA
CCTGGTGAAACCCTGGCTCTACAAAAATACAAAAATTAGCCAGGTAGGGTGTCATGTACCTGTAGTCCAGCTACTCAGGAGGCTGAGGTGGGAG
GATCACCCGAGCCTGGGAGGCAGAGGTTGCAGTGAGCCGGTATGGTGCCACTGCATTCCAGCCTGGGTGGCAGAGTGAGATCATCTCAAAAAAA
AATGTTTTTTAAGATTTATTTTTGTAGTTATTGGAGGCAGAGGAAGGAAATGTGAAAGATATTTTAGATGCTAGCAAAATTGTAGATGTGAAGA
ACATGAGCATAAGACAGCATGAGTATGGAGACAGGCAGACAAAGTAGCCTCCGGTTGCAAGTGCCATGTGAAGTTTGAACAATAGTAATGTTAT
TATAATTGAGTTTTTGCTTCCTTATTCACGCCCACCCAATTTTACTGATGTCATGGTTTTCCATGTGGGAACAAGTATGACTGGTGGTGGTGGG
GGAGTTCCCCCCACCATCGTCTATGCTACACATATGCAAATTAGTAGTATTTGTAGCTATTGGGAAGTTTTTCATAAATGACATTTTGAAACTT
GTTAAATATTGTTAGCATTAAACTTCTTTCTAATACAAAAACATTTTAGTAATTTTTTATGATGAATGGCTATAACTTTATATAATGTGGCACA
AAACATATTCTACTGCTAATAACAGTGAATTCAGGGGCTCAAAAAGCAATCTCTGTGGAGCTAGGCAAGCAGCATAAATGAGTGGTGTGGATCA
AGTGTAGGAGGACTAACAGGCACTGTAGCAAACTAAAGAGCCCTGAATAAATGGGGCTTAGTCCCTAGCCTGTTGTTTCTGTGCCAACCTAGTA
TTGCCAGGTCTTTAAAATGTACAAGAGAAGCCGGAAATCCAGATGTTCATATGCAATTACCTGATTTCTAAATGTAGATAACCTGTGCAGATTT
TTAAAAATATAGGCAACACTTTTGTGAGCCTAGCAAAACATACCTGTGGGCTTCAACCTGTGAACATCTCTTTACAGCTTTTGCTTTACATTAT
CAAACAGACTTTATTGATTTGATTCTGAATTTGTTTGGGAGGCAAATTTTACCATTTAATATTGCTTTACTGTTAATCTATATTTTGGTATGTT
TGACATAGGCATATTAGTAATTGTAAAACACTGTATGTTAAAATGATATAGTATATATTATTTTTCTTTTAACAGTTCTTAGAAGTACAACCCC
ACTTCTTTTTTTTTTTTTGAGGCAGAGTTTTGCTTTTGTTGCCCAGGCTGGAGTGCAATGGCGTGATCTCGGCTTACCGCAACCTCTGCCTCCC
AGGCTCAAGTGATTCTCCTGCCTCAGCCTCCCGAGTAGCTGAGATTACAAGCATGTGCCACTATGCCTGGCTAATTTTGTATTTTTAGTAGAGA
CGGGGTTTCTCCATGTTGATCAGGCTGGTCTGGAACTCCTGACCTCAGGTGATCCACCTGCCTTGGCCTCCCAAAGTGCTGGGATTACAGGCAC
AAGCCACGCGCCCAGCCATATAATCCCACTTCTTACTCTCTCATCTTTATCAGAGCAATTGGAAGCACGTTCATGCTTTGGTATGTACAACTGA
CAGCATGATAACTTCAGTATTGCTATATGTTTGATTTTTGCAATATTCCTTGTGTTTATAATGCAGTTTTGAATATTGTTTTTTTCCTGCTAGT
TTCCAAAATTTGGGTATTCGATGTGTGAAGAAAAAAGAAGTAAAAGAAGCTATTATTACAAGAATAAAGGCAGGAATCAATCCATTCAATGGTA
AGTATGTTTGATAAAATCATTTCTATTTATTGGGTGTTGATTTCTGTTTCTTTTTTTCTTCCCTTTTTTTTTTTTTTTTTTTTTTTGAGACGGA
GTTTTGCTCTTGTCGCCAGGCTGGAGTGCAATGGCGCGATCTCAGCTCACTGCAACCTCCGCCTCCCAGGTTCAAGCAATTCTCCTGCCTCAGC
CTCAGCCTCCGGAGTAGCTGGGATTATAGGCGCCCACCATCACACCCAGCTAATTTTTGTATTTTAAGTAGAGACGGGGTTTCACCATGTTAGC
CAGGCTGGTCTCGAACTCCTGACATCAGGTGATCTACCCGCTTTGGCATCCCAAAGTGCTGGGATTACAGGCGTGAGCCATTGTGCCCAGCTAT
TTATATCTTCAACCTGTGTTTTAAGTTCTGTGTTTACTTTTCTACTTGTTGACATGTACCTGTATCTGAGGAATGACGTATACTATTTAAAATA
GTTTCTTCACAGTTGTTGAAAAAAATCTTATGCACAGAGAGGTTTTTGGTATTGTTGGAGAGTGTCACTGTTTTATATAATAAAACTTCAAAAT
GTTGTGATCCTTTTGAACATGGCCTTCCTTATGCCACTTCCAGCAGACAACATACACTCCCCCCTTTTGAGTGTTAAATAGGCACTGTGTTTAT
AAGGTATTGGAGTGCATTTAGCTCAAGGGTAACACTTAATCCCAATGTGGAAGAAGAAGCATAGAAATAATAGAAATCACACAAAGTATTTTCA
TAGCATATTTGTGTATATGACTAGATATTTACTGTTCAGTGAATTTTATTGGGGATAGCATTTATTAAGTATTTATACTTATGGTACTATCTGC
CTCATGCTGTAAGTATAAATGCTTAATACTCCCTGGCTAACTGTTACTTGTAAGCACTCTGTATTGTTTGAATAGATAAGTACTACCACACTAG
ATGTGGCATCCTATACTTAGAGTAGCCTCCATTCTGAAGTCTTTGCAGCTACTTAAATATGCAGAAGTCAGGAATTAAATGACTGTAATATTTT
TCAAAGAATATATTTCTTGGTCATATTTGAGACATCCTTATACTGTTGGTTTGTATTTTTCCTGGATGTATAAAAATAATTTAAGAGATATGTT
GATAATTTAAGGAATATATTCACAGGTAATTACTGTTTTATGAACCATAAGATCTGCATCTCTTTAGGTTTTATTTTTCACACTATTTAGTATA
AAGCGGAAAGTAAAAGTCACCTTCTTCCCTTCCAGTCTTACCAGGGGAGACTGCTATTAAGTTTGATGTATATTTTTCTAGAATTTTCTTTATA
CATGTGTAGTTATATGTAGACATATATATTTTATAAAAAAGGAATCATGCTATATATCCTATTCTACTCCTTCATTTTTTTCCACTCAGGAATA
TACCTTAAACTTTCCATGTCAATACTTACAAATCTGTTTTTTTTTTTTAACAGAATATTTTATTGTTTGGGTAGAACATAATTTGTTTATTTAG
TCATCTTTTTTTTTTTTTTTTTTGAGGTAGTCTTGCTCTGTCGCCCAGGATGGAGTGCAGTGATGTGATCACAGATCACTGCGGCGTTGACATC
CTGGGCTCAAGCGGTCATCCCACCTCAGCCTCCCAAGTTGTTGGTGCACCACCATGCCCATGCCCAGCTAGTTTTTGTATTTTTTGTAGAGACA
GGGTCTCGCCATGTTGCCCAGGCTGGTCTCAAAACTCCTGAGCTCAGCAATCCACTTGCCTCGGCCTTCTAAAGTGCTGGGATTACAGGCATGA
GCCACTGCACCCGGTCTATTTAGTCTTCTATTAATGAACATGTATGTTGTTTTCATTTTGTTTTAAAAGCTCCACTGAATATACACTGTGAGCT
CTTGTGTGATTATTTCCCTAGGATAAATTTTTGGAAGTGGAAAATTTCTGGGTCAAGAGTATGCTCATTTAAAATTGTGATTCATTGCCAAATT
AATAGGCCAGACTTTTAAAGAGGCAGGTAAGTCTAGATGCAATATAAAACAAGGAAATTATAAGTGATAGCTATCATTTATTAAGCACTTCTTG
TATGTCAGGGAGTAAACGTTAGTGCTTTATGCACACTACTTTATTTAATCGTTGGAACAATTCTGCTTTATGGAAGAGAATGCCAAGACTTTTG
TTATTTAACTGTCCCAAATCATGCTGCAAATATAAGTGGCAGACCTGGAATTCATATGCAGGTTCCTTGACTCCAGTGGTAAAGGCATTAAAAA
GGAGATGCTGTCTGTGGAATTTCTTTTTCTTAGTTTTGGCAGCTCAGAATCAGGCTTGTTTTTTTGAGATGCCCGGGCTGGAGTGCAGTGATGT
GATCACAGCTCACTGCAGCCTTGACCTCCCAGGCTCAGGTGATTCTCCCACCTCAGCCTCCTAAGTAGCTGGGATTACAGGTGTGCATCACCAT
GCCCAGCTAATTTTTTTTTTTGAGACAGAGTCTCACTCTGTCACCCAGGCTGGAGTGCAGTGTCACAATCTCGACTTGCTGCAACCCTCACCTC
CTGGGTTCAAGCGATTCTCGTGCCTCAGCCTCTCGAGTAGTTGGAATTGTAGATGTATACTACGCCTGGCTAATTTTTGTATTTTTATTAGACA
CAGGGTTTCACCATGTCAGCCAGCCTGGTGTTGAACTCCCAACCTCAAGTGATCCTCCTGCCTCAGCCTCCCAAAGTGTTGGGATTACAGGTGT
TAGCCACCACGCCTGGCCAGAGTTTCTGTATTTTTTGTAGAGATGGGTTTTGCCATATTCTCCAGGCTGATTTCAAACTCCTGGGGTCCAGTGA
TCCGCCTGCCTTGGCCTCCCAAAGTGCTGGGATTACAGGTGTGAGCCACTGGGCCCGACTCAGGCATGATATTAATAGTGAATTCTCATAATGG
CAATTAAAATAGTTGTTGGTAGATAGTAAGTGCTTTATATATTTTATTTCATTTAATCCTTATAACAGCCTACGAAATAGGTACTATTAATAAC
TCAATTTTAAAAATAAGAAAATGGAAGCATAGAAAAGTGAACTCAGGCTTGTAATCCCAGCACTTTGGGAGGCCGAGGTGGGGGGATCACGAGA
TCAGGAGTTCGAGACCAGCCTGACCAACATGGTGAAACCTTGTCTCTACTAAAAACACAAAAAATTGGCTGGACGTGGTGGCACGCGCCTGTAG
TCCCAGCTACTTGGGAGGCTGAAGCAGGAGATTCGCTTGAACCCAGGAGGTGGAGGTTGCAGTAAGCCAAGATTGCTCCACTGCACTCCAGCCT
GGGCGACAGAACGAGACTCTGTCTCAATTAAAAAAAAAAAAAAAATTAGCCGGGCGTGGTGGCGCACACCTGTGATCCCAGCTACTCAGGAGGC
TAAGGCAGGAGAATCGCTTGAACCCAGGAGGCGGAGGTTGTGGTGAGCCGAGATCACGCCATTGGACTCCAACCTGGGCAACAAGAGTGAAACT
CCATCTCAAAAAAAATAATTAAAAAAAAAAAAAATCAAGTAGGCCAGGCACGGTGTCTTATGCCTGTAATCCCAGCACTTTGGGAGGCCAAGGT
GGAGAGGATTGCTTGAGCCCAGGAGTTTGAGATCAGCCTGGGCAACATGGTAAAACCCCATCTCTACAAAAATTTTTTTTAATTAACCAGATGC
AGGGGTGCACAACTGTGGTCTTAGCTACTCAGGAGGCCGAGGTAGGAAAATTGCTTCAGCCTGGGAGGTCAAGGCTGCAGTGAACGGTGTTCTC
ACTATTGCACTTCAGCCTGAGCGACGGAACAAGACCCCGTCTCTAAGTAAAGAAAAGGAAAGTAATTTGTTCAAGGTTATATAGCTGATGAGTC
GTAGAGCTAGGATTTGAACATAGGGAGCCCCTGACTCTAAGGTCATGGTTTTAACAACTCTACCATGCCGCCTGGATGAATATTGATTTGGGGA
AGGAACAATCCTGCCAGTTAATCCTTCCCACTATTATGTTTCCTGTTGTTGTTTACTTGTTTTTTCTTTGTTTTGTTTGTGATGGAGTCTCCCT
CTGTCGCCAGGCTGGAGTGCAGTGGCACGATCTCGGCTCACTGCAAGCTCCGCCTCCCGGGTTCACACCATTCTCCTGCCTCAGCCTCCCCGAG
TAGCTGGGACTATAGGCACCCGCCACCGCACCCGGCTAATTTTTTTTGTATTTTAGTAGAGATGGGTTTCATCGTGTTAGCCAGGATGGTCTCA
ATCTCCTGACCTTGTGATCCGCCCGCCTCAGCCTCCCAAAGTGCTGGGATTACAGGCATGAGCCACCACACCCAGCCTGTTTGCAAATTCATTC
CTTCCCGAGCCTTTCAGCAGTGGTTCTCAATTTGAATGTGCATAGGAATATCTTAGTACTGTTAAAAATGTAGATTTCAGGCCACACTTCCACT
AATTTGATAGGGCTGGTATGGGTCCTGGGAATCTACATTTTAAAAAATATTTTTAAAATAATAACTTATCCGAGTCACATGGCACCAAAGTGTG
TTAGCAGCCAAAAGTATCTGTTCGGGTCTGCAGCAGCCCCAATTCTTGCCTCCTCAGGAGACAGAAGGAGAGACCGAGACAAGTTTTAGAGCAG
GAGTGAAAGTTTATTAAAAAGCCTTAGAGCAGGAAGGAAAGGAAGAAAAATACACTTGGAAGAGGGCCAAGCAGGTGACTTGAAAGACAGGTGT
GCTGGGAATCTGCATTTTAACAAACATCTAGATCAGTATTTCTTAACTCTTTCACCATGACCCACAGTAAGAAATAGATTTTGTTTTTGAGTTC
AATATATATACATACACCACACCCTTAGGACAGCGATACCCTATGTATTAGTCCGTTTTCACTCTGCTGATAAAGACACACCTGAGACTGTGAA
GAAAAAGAAGTTTAATTGGACTTACAGTTCCACATGGCTGGGGAGGCCTCAGAATCATGGCGGAGGTGAAAGGCACTTCTTAGATGGTGGCAGC
AAGAGAAAATGAGGAAAATGCAAAAGCGGAAATCCCTGATAAAACCATCATATCTTGTGAGACTTATTCACTACCACGAGAGCAGTATGGGGGA
AACCACCCCCATGATTCAAATTATCTCCCACTGGGTCCCTCCCACAACACATGAGAATTATGGGAGTACAATTCAAGATGAGATTTGGGTGGAG
ACACAGAGCCAAACCATATCGCCCCACTACATGCAGTGTACTCAATTCTATTCTGTACCTTTCCTTTCCCTTCTTTTTCTTTCTCTCTTGCATT
GTTAGTGCTTACCCAGCCCACTAAATTAATTTTACAATCAGTGGTTCCCAAGCTACAATTTGAAAATCTCTATCTGAGATGATTCTGATGCAAG
TGATCCACAGAGTATACTCTGAGAAACTTTGATCTGATCTACATGCTGAGTTCAGCTTCTCAAATATGGCCTTTAAAGCCCTTCATAATCTGGA
AGCCTTCCTTTCCATTCTAGCTTTCATTCTGTACCTCAGCTTCCTACCCCTCCTTCCTCTACCACTGAGTGCACACATGCACTTACTTGTTTTT
GCCGTTCTGAACTATCTGCACTGTTAGAAGAGCCATGTTCTTTCTTACCTCTGTGCTTTTATACATGCAGTTCCTTTGCATGAAATACCCTGAT
CTGCCTGGTGAATTTTTCTTACTCACCCTCCAAGTTTAAGGATCACATCGTCTTTAAAGCCTTCCCTGACCTTCTCTCTGTTTTCCCAAAGTAC
CTGTGTGTGTGTGTATATATATATGTGTGTGTATGTATGTGTGTGTGTGTGCGTGTGTGTATGTGTGTGTGTGTATATATATATATATATATAT
ATATTTTTTTTTTTTTTTTTTTCTTTTCCGAGACAGAGTTTCGCTCTTGTTGCCCAGGCTGGAGTGCAATGGTGCAATCTTGGCTTACCGCAAC
CTCCACCTCCCAGGTTCAAGCAATTCTCCTGCCTCAGNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNCTGGAGTGCAGTGGTGTGATCTCGGCTCACTGCAAGCTCTGTCTCCCAGGTTCACACCATTCTCC
TGCCTCAGCCTCCCGAGTAGCTAGGACTACAGGTGCCCGCCACCATGCCCAGCTAATTTTTTTGTATTTTTTAGTAGAGACAGGGTTCCACCGT
GTTAGCCAGGATGGCCTCGGTCTCCTGATCTCGTGTTCCGTCCACCTCGGCCTCCCAAAGTGCTGGGATTACAGGCTTGAGCCACCACGCCCCG
CCTTATTTGTACTTTTCTACCAAAAATATCACAGTTGTTTTCCTTACCTGAGATGCCCTGAAAGCCTTCTCAGTTCTCTTAAGTCTGAGATAAC
ATCTTCCTAATTATACGGTATACTCAGCAATTAATCATTGAAAGTTTTGTGTTATCTGTTAGATCATTTGATTCTTCAGGAATTTTCTTAAAAT
ATTAATTTCATGCTACTTGTGGACAGGGTCTGTGATGGAATCTTGTTTCCATTTCACTTTGGCACCTAAACAATGTATTTGTTTTACTGTTATC
TCTATCTTCTCAGCTAAGTGCCTTGAGCTATTGAGTTTCCTACCTGCACACAATAGATTCCCCAACTAGTGTGTTTTAAGGCCGGCCTTTATCT
TTCTTGCCATTTGTTCTTTAAAACTAAGAAACAGTCTCTAGCAAACGGGGCATAAAGCAGTCTAATACTGTCAAATTTGAGGCTTCAAGTAAAG
GATTATTCAAATTGTCTAATTGCCAAAGTCTAAATTGGGTGAGGAATTTAATTAGGGACTCCAATATCAGATTTTAACAGTGTTTACTTAGCTA
GAACAAGAGTGGCAGTTAGACTTTGTAGAGGACTCTTAACTGCAAAAGCTGAAAAAAATTTGTAATTATATATCATTTTCTAGTTATGTACTTT
GGTAATGAAATGTTAGGATTTCTAGTATGCGGCAGTTAAACTCCATAGAATATCTAAACTGTATATTAAATTTTGTGCAATAAAATTGATTTTG
ACAATAAAGAATTTGCAATTAAGTTTTATTAGGTAGTTTACTGTTTTCCCATGTAGAGAATCTTGAGTTGTTATGAGAGTAAAAGGCTGCTATA
CAGTTGAGTTTGGGAAAACTACATTACTTAGTAGTCTGAAAGGGAATACAGTTTAATAAGTAGTCTAACATAGATTCTTATCTCTAAAGTCTGG
TCTAATACAAGAAAAAGCCTTTCCTTACAACTGAAAACTTAAAGTTTGAAAAAATGGCTCATGTGTACTTCATTGTCCTTTCTTTATTGCAGTC
AGAGTTTTCGAAGCCTTTTTTTCATTACCCTAGGCAGAACTTGTGTACTTTCTTGCTTTCAGTATTCCTTGAGCATATTTCCATTATAAAGTTT
TTCATGTTGTATTATATAATTTTAGTTTATGTATCTATTTCTCATCCAGATACTCATCTCACTAAGGTCAGGCATTGTCTTTTCAAATTTGTAT
CTAACACTTTACTATGGTAGCACATAATAGATACTCAATAAGTATGTTGAAAGGTTGAGTAAATTAATGATATACTTCTAGTATACTTCTAGCA
CCTTTACTGTTTGCCTGGCACATGAACACTTAAATGTCAACTTTTATTATTATACATAGGGGATACTTTATATACTTTTTAAGTAACAAAAATG
TAAATTAATTATAACTAAATTTTATATTTTTCCTTAAGTGAGACTTAGTTCATTTTTTTTTTTTCTATTATTTGTAGAGACAGGGTCTCCGTAT
GTTGCCCAGGCTGGTCTTGAACTCCTGGGCTTAAGTGATCCTCCTGCCTCGGTTTCCAAAAGTGCTGAGATTACAGGCATGAGCCACCATGCCC
GACTGAGACATAGTTCTTAAAATTGTTATTCCAGGCTGGGCGCAGTGGCTCACGCCTGTAATCCCAGCACTTTGGGAGGCCGAGATGGGCGGAT
CACGAGGTCAAGAGATACAGACCATCCTGGCCAACATAATGAAACCCTATCTCTACTAAAAATACAAAAATTAGCTGGGCATGGTGGCGTGCAC
CTCAGTCCTAGCTACTTGGGAGGCTGAGGCAGGAGGAGAATCAGTTGAACCAGGAGGCGGGGGGTTGCAGTCAGCTGAGATGGCGCCACTGCAC
TCCAGCCTGGCGACAGAGCAAGACTCCATCTCAAAAAAATAAAATTATTATTCCATATGCTGTGTATTTTTGCCTTGTTATTTATTTTTTATTT
CAGTAGTGTTTGACAGTATTAGAATTTTAGAAATGTTACACTTCTAGAGCTAGTAGACATAATTGATTTTTAAAATTTAATTCCTGCTTATGAA
AAATATAGCATGATAATATCTTCTTGGTTAAGAATGAAATTCTCATTACACACCTACTGATTTTAGGGTTAAAGGAGTGATATTTCTTTCTAAA
GGTAGATTAAACATTTAACATAAAAAGGAAGGGGCTGGGCACGGTGGCTCACACCTGTAATCCCAGCACTTTGGGAGGCTGAGGTGCGCAGATC
ACGAGTTCAGGAGATCAAGACCATCCTGGCTAACAGGGTGAAACTCCGTCTCTACTAAAAATACAAAAAATTAGCCGGGCATGGTGGCGGGCGC
CTGTAGTCCCAGCTACTCGGGAGGCTGAGACGGGAGAATGGCGTGAACCCGGGAGGCCGGAGCTTGCAGGGAGCCAAGCTCACATCATGCACTC
CAGCCTGGGTGACAGAGTGAGACTCCATCTCAAAAAAAAAAACAAAAAAAAAAAAAAAAAAACATAAAAAGGAAGGAGAACAGGAGAACAGTTA
TTACCTTATATTTTGACTAACTACAAAGTGAAATTTTAAACTAGTTTAAGAGGAATTTGAGAGAAGTCGATTGGTCTCATGCTTCCTTCCGTTA
GTTTCTGCAAATGTTAAAACTGAGTATCAGGCCAGGCACGGCCACTCACACCTGTAATCCCAACACTGGGGGCCCAAGGTGGGTGGATCACTTG
AGGTCAGGAGTTCCAGACCACCTTGGCCAACATGGTGAAACCCCGTCTCTACTAAAAATACAAAACTTAGCCAGGCATGGTGGTGCATGCCTGT
AATCCTAGCTGCTTGGGAGGCTGAGGCGTTAGAATCTCTTGAACCCTGGAGGCAGAGGCTGCAGTGAGCCAAGATCACAACACTGCACTCTAAC
CTGGGCGACAGAGTGAGACTCTGTCTCAAACAAAACAAACAAAACTGAGTATCAATTTTGACATTACAGAATAAAACACTGCATTTTGTATTAT
AGAATGATATGTATCTATTAAAATGCCTACAGCAAGCATTATCCTAAATAAGAGAAATATCAGAAACATTCGCTTTGAAGTTAAGAATGAGAAA
CCAAGGCACACTTTTACCACTTGTATTCAACATCGTAGTGAAGGTCATAGCCAGACAGTGAGCATTGTAGCCAGTGTACAGTAAGACCCCCCCA
AAAAGTAGAGGCATAGGATTGAAAAATGAGGAAATAAAACTATAATTATTTGAAATTATTATAGTGATAACCCAAAAGATATATAAATGGATTA
TTTAGATAAGTTTAAAAGAGTGAGCTTTGTGCATTGGCAGTATAGCCAAGTGAGGTTTATCTTAGGAGTGGTTATTGTTAATTGAAAACTTTTT
CCTAATATCCTGCTGTCATGACTTGCAGTATAGTCAGCATTGGCAATTTTTTGACAGTCTTTTTGGAGACTGAATAAAAATAAAGTTTAAAAGG
GTTAGTGGATGTAAAATTAATATATAAAGGTCAGTCATATTTCCATATCTCAGCAGCAAATAACTGGAAAACATCATTAAAACAAATATTGTCA
GGAGATATGAAGTATGTAGAAACAAATCAAAGAAATGATAAGATTTCTTTAGGGAAAATTATAAAATTTTATTATGAGATCTTAGAGAATACTT
AAATAAATGGAGAGGTATTCCGTGTTCTCTAATAGTAATATTGTAAAAATGTCATTTTCCCAGGCCGGGCGTGGTAGCTCACACCTGTAATCCC
AGCACTTTGGGAGGCCGAGGCGGGCGGATCACAAGGTCAAGAGGTTGAGACCATCCTGGCCAAGATGATGAAACCCCATCTCTACTTAAAATAC
AAAATTAGCTGGGCGTGGTGGCACAGGCCTGTAGTCCCAGCTACTCGGGAGGCTGAGGCAGGAGAATCACTTGAACCCGGGAGGCGGAGGTTGC
AGTGAGCTGAGATCGTGCCACTGCACTCCAGCCTGGGCAAAGAGCAAGACTGTCTCAAAAAAAAAAAAAAAAAAAAAAAAAAGTCATTTTCCCC
AATTGATAAGTAGATTCAATGCAACTCCATATTAAATGTGATAATTCTAAACAGGAACAGAATATAGAAAAAGAACAATGAGGAGAAATTGACC
TCATCAGATAGAAGTATTAATACTAATTAAGGCAGTGTCATATTGGGATAAGAATAGATAGATTGACTCATTGGAGTAGAATTGAGAGCCCAGA
AGAGACTCCAATCACCTGTGACCCTGAACTGGAATAATTGGGTAAATCATTATCTTAACTTGTTTTTATTAATCTTTCTTAAATGTATGTATAG
CTCAGATTTATTTCAATATTTAATATTGCAAGCGTTTTGGTCTTATAAATTTAGTGATGTTTTTGTGACCCATAGTCTGCCGTAGAAACTTAAC
TCTTATTAATATTGATTAGCCTATGGTAAAATTGGTTTTATTATGGTGTTGTTTCGCTTGAAGTTGCAATTTCCAAGAACCTATAGACATCATC
CTAGAAGACATTAAGTGAAGACTTACTGTGTATATAAGCAGATTTGAAATTAAAAATATAAAAGATCTTCTGAAGTTGCAACAGTGCTCAGAAG
TATTATAAAATAGAAATAAATCATTTCCAAAAGAGAGATTTCATTTTTGCCCAAAATCTTTCTTCTAAGCTATAAAAGACAAAACTGTTCAATA
CACAGTTTTGTCAATTTAAATCTACATATTCACCCAATTTTTATAAGATAAAATAAAAATGTATAATTATTGAAAAAAGAATTGAGAGCCCAGA
AACAGAGCTAAGGAAATATGCGACTTTAGTGTATGACAGAGGTGGCATGTCATATCAAGAGGAAAGGAGGAAATGTTTGAGAAATGAAGCTTGA
AAAAAAAATAGTTATTTAATTAAGTCGGATTCCCTACCTCACATCATATACAAATATCAGCTGAAGATGGATTAAAGGCTTTAAAAGTGAATCA
ACATTTTAAAAACTTTTGGTAGACTATGTTGGTAAACATTTTAATCCTTGAGGTATAAAAAGACAAGACACAAAGTGTTTTCTATAAAACTTTT
GATAAATTTGATTATATTCTTAATTTCAATTTATTATATTTTTCAGTTCTAGAATTTGATTCCTTTTTTTCAGTTTCAAGTTCTCCAGTAAACT
CTCAATCTTAATTCCTTGAATATATTAAGTATAGTTACTTTAAATTATATGTCTGAAAACTTCATTATTTGGATCCTTTGTGGTATGTTTTTAT
TGCCTGTTGCTTTCTTTTAAAAAAAAATTTTTTATTTTGCCTCCTCATATGTGTGGTTATTTAAGATAAAATACCAGTCATTTTCTGTGAAAAA
GAGTAAAGATTTTAGGCCTACAATATTATCTCCCTGCAGAAAATTTTCTTCTGGCTAGTGGCCGGGTACACTAGCAATCCCAGGTCACCTAAAA
TCAGTGAATGATTTTAGGTTATTTGAAGTTGGGCTTTCATCCCTGAAATCTGATATGTTTCCAGTTCACCCTACTCCTAGGGTATAGCTCTTTC
AGACCTCAAATCCCCCAAAGCCTTGGGTATTTATCAGATCCCACATCCTTGGCAAACTCTCAACTCCTTTATTTTTTCCCTATAGTTTATTGAG
AGTTCATCTCAATCTTCTTAACCATCTCTTAGAAAATTGTCAGGCTATATTCAGATAAAAGTGGCCTCTCATTCTGAACTGTGGGTTTTCTTCT
CTCATATCTTGGTCGCATGGTTTTTCATGACCTTGTTAGCTCTCTAATGTCATTAGAGAAATTTTGTTTCATATTTTGTCCACTTTATTCTCAT
GGTTGAATTGGTCCAGTTTACAGATTATCATCTATCATTACCAAAGTAGAACTTCTTTTTATTAACGTTTACTTTATGCTTTCTTCAGTAACTT
GTAAAAGTATCTAAATGTTTGGTTGTTCAATAGGACTTTAAGTAATTCTTCAGTCATTTATGTTAATTGAGAAAATGTATAATAAATATCACTA
CTAGGTTCAACACCTAGTGGAGATACATCAGACATCCCTTCTGACTTCATTTGAAACTTTGTCTAATAGGATAGAAGAGGCATACATAAATGAT
TATCATGTAAGATTTTAAAACTTTTAAGGGCATAAGAAAGGTATAGGTCAAGTCTTGCACTGATTTAGAGAGGAGAAATATTCCCTCCATTGCA
GAGGATGAAGTTAGGCTTTATAGAAAAGAAGCTTTGAGCTGGGGTTTCAGCCTTACACTGAATCTTGCATTGGGTAAAAACGGGTATCTGTCAT
GATTCTTGCCTGTCAGTAGTTTTTAACCATGTTGTGCAGAGATGAAAGTAATTCTGATGCAGTAGTAATTGTATTTCCACACATTGAGAAGATT
CAAAGCAAAATCTCATAAACAGTGATTTTTTTTGAGAAGTAAGTAGAATTTCTTCCCTGTAGTAGAACTATTATGTGACTAAATGTTTTTGAAC
TTTGGCTTATTGACCAGTTTGTTTATTTTTCTTTTATGTGCCATTTAAAAATATTTAAAATAAACTTTTATTGAGATACAATAAAATCTACCCG
AATATGTCCTGCTACATAACCACAATCAAGCTATGGAACATTTCCATCACCCCCTAAAATTTCCCTGGTCCCCTTCTCGCTCAGTTCCCTACCC
TGCCTCTGCCTCAGGCAATTACTAATCTGCCTTCTGTCACTGGAGATTAATCTTTTCTAAAACTGCATGTAAATGGAAGCATACAGTATACTTT
TGTATCTGGTTTCTTTTGCTCAACATGGTTTTGAGATACATTCATTTGGGAGTGTATACCCATAGCTTGTTTTTTTGTTTTTTTTTTTTTTTTT
GAGATGGAGTCTTGCTCTGTCGCCCAGGCTGGAGTGCAGTGGCACAATTTCGGCTCACTGCAACGCTCTGCCTCTGGGTTCACCATTCTCCTGC
CTCAGCCTCCCAAGTAGCTGGGATTATAGGCGCCCGCCACCACGCCTGGCTAATTTTTTGTATTTTTAGTAGAGGCGGGGTTTCACCGTGTTAG
CCAGGATGGTCTCGATCTCCCAACCTCATGATCCACCCACCTCGGCCTCCCAAAGTGCTGGGATTACAGGTGTGAGCCACCACGCCTGGCCAGT
TTGTTCTCTTTTATTGCTGAGTTTATTCCATTGTAGGTCTATACCACACTTTGTTTATCCATTCACAGAGTTCACATAGGTTTGTTTTTAAACT
TACCAGGAACATCTAGCTACAAATTATCTTTTTTTGCATATTGATTACTCATCTTTGCAATTGAGAGACACTAATATCTTGAATGCTACATTTA
TAATGCTGTAAAAATGGATTTTGAAGAGGTAGCTCTCATATTGCCTACACAATAGTGCCAGTAGAGTATGCTGCTGTAACACAAGTGTTGGTAT
CATAACATTTTGACCTTTCAGTTGTACAGAGTACAGATGTGTTTTTATGCACTGTTTCAAAGATTAAGCATAAGAATGCTAATACTGGGATACT
TATAAATGTTGTGATAGCTAAGTAGACTCTCAATGTCATTAAATAAAAACAGTACAAAATACATTATCAGTTTTATTAGTTATTGCCTGTAAAA
TAAGGAATTTGGGCTTAACTATGTTTCTAAATTTAGTGTTTCAAAATTTAATACTTTGCAAAGTAAAATTGTTATGTAAATAATGTGCATGTCT
TGCTTTTTAAAAATATGACTTATTGCTATAGCTTATGGCATTTGAAAAGGCAATATTTTCTTTTGGAACTGGATTTCTGGTTCAAATACTAATT
TAACAAACTATTATACTATATTCTATCATGAGCTTTTCTAGTTTTATAGAACTGTAAAATTTTTAATTAAAACATTTTTTAAGTTTGCACGTAA
AATTTCTTATGGAAACTAGCTATCTTAACATTTTTTTGTTTCTTGGTATTTCATCCTTAAAATGTTGATCCACATTTAGTAGATACTCTTGTGT
ATGTCATTTTTATGTACAACACATGTACAATTTTCTAATAGATTTCAATTTTTAAAAGCATAGGCTCTAAATTTAATGACTTGAGTTCACATTT
CCACCCTGTCACTGTGAAACTTTGGGCAAGTAATTCATCTCTTTACAGTCAGTTCCAGGCCAGGCTCAGTGGCTCACGCCCATAATCCCAACAC
TTTGGGAGGCCAAGGTGGGTGGATCACTTGAGCCTGGGAGTTTGAGACCACCCTGGGCAACATAGTGAAACCCCATCTCTACAAAAAAATAGAA
AAATTAGCTGGGCGTGGTGGCATGTGCCTGTAGTCCCAGCTACTCAGGAGGCTGAGATGGGAAGATCACTTGAGCCTGGGAGGCGGAGGTTGAG
GTGAACCAAAATCGCATCACTGCACTCCAGCCTGGGCAACAGAGTGAGACCCCCATGTCAAAAAAACGAACTAAACTCAGTTCCTCGTTTGTAA
AATGGGGATGAAAGTAATTCTTGTACAGATTTAGGAGATAATTTACAGAGTCCTTAACACAACAACTGGACCAAATACATACTTGTTGAATGAA
TGGGTAAATGCCATTCTTTGTTGTGTACAAACCTGCAACTATTTTTTAAAACTTTGATTTGTTATTAATGTCAACTTGGGATTTTGCTTTAAAT
CCCAGGTGAGGGGATAGTTACTTCCTGAACATGGCTATAATTTATCAAATACTGTTAGATAGTGATTACTTGATAAGAATGATAAAAGGCATTC
TTGAGATTGTATACTGTTCTATTTTTATTCATACATATTTGGATGCTATTCAAGGTTATTGGCTATAATGGGCAGGGAGGAGGACCTAGCAAGT
CTTTAGGTCTATGTGACTATTACATTTAAAAAATTTTTTTTTTCTATTCAGTCCCTGAAAAACAGCTGAATGATATTGAAGATTGTGACCTCAA
TGTGGTGAGACTGTGTTTTCAAGTTTTTCTCCCTGATGAACATGGTAATTTGACGACTGCTCTTCCTCCTGTTGTCTCGAACCCAATTTATGAC
AACCGTAAGTACTTCATTTTCTTATATTTGTAGTCTTAACTTGTTTTTTGTAATAGTTTAATTCTTAAAATTATTTTGGTAATTAGGAAAACAA
TATATTCATGATAGGAAAACTTCCAGACATTAGAGAAATATATAACAGAAAGGAAATCTTCTCTAATCTCATCCTCCAGAGATAATCACTATCC
TTAGGTGCATATTTTACCAGTTTCTTCTTCCTGTATATACACAGATACATTGTCATCATCATCATTTTTGACACAGTACAATTTTGGCAACTTA
AAATCATAAGTGATGATTGTTTTAAAAACTAACTTTCTTCTTACCATATATCTTGGACATTTTCCTTTTAATCACCTTATTCTTTTCAGTAGCA
GCATATTTTATTATGTAGTTATATCATAATTTTATTAATTACCCTCTTACAAGGCATTTGGTTTGTTTCTTTTCTCATTTTTTCTTTTTGGCCT
AGCAAACTGTACTGCAATACATATTCTTGTATATTTTTTTTTATTGGTTTTTTTTTTTTTTTTGGAGACAGGGTCTCACTCTGTCACCCAGGCT
GGAGTGCAGTGGTGCAGTCATGGCTCACTGCATGGCAAATGCTGGCTCAAAGGATATGGATATATCTCATTTTGAAAGATTATGAAACTTTCCC
CCAAAATACTGTGTGTGTGTGTGTGTGTGTGTGTGTGTGTGTATGTGTGTGTGTGTGTACATAGACTTAGATATACGTCCACCAATGGTGTATG
ATATACCTGTTTTATGTGTAGAAGGATAGAGAGACTTAGGGCTATAAAGGAAGCATGAAAGTTGTATTGCCATAGGGAGATGTATTGCCATAGG
GAAAATTGTATTGCCATGGGGATGTGGTCATGATTTATGAAGATTAATCTGGTGACAGTGGGTAGGATTGATTGGCGGAATCAGAATTTAGAAG
CAGGGAGATGTAATTAGAGAATATGTCATTACCTAGAAATGAAGCCACAAAGTCTAAAGTAAAGCAGTTAGAAAGGAAGTGGACAGATAAATAG
ATGATTAATGTATTTAGTGTCATTTATCTATACACTAAAACTTTTATTCTGTGAATGCTTTTCCTCAAATTCTTCCCTGCAAAAAGAAATAAAA
TATTACTAAGGTAGCAACTCATTTTTTTGAAAATCCTTTATATTTAGGTGCTCCAAATACTGCAGAATTAAGGATTTGTCGTGTAAACAAGAAT
TGTGGAAGTGTCAGAGGAGGAGATGAAATATTTCTACTTTGTGACAAAGTTCAGAAAGGTATTTATTTATTTCATTGAATTTAGAATAAATTTT
AGATTAATAGATGCAGTTACTTTGTTTTCCCATTTTTTTTTTTTTGGTTTCTTATTGACTAGATGACATAGAAGTTCGTTTTGTGTTGAACGAT
TGGGAAGCAAAAGGCATCTTTTCACAAGCTGATGTACACCGTCAAGTAGCCATTGTTTTCAAAACTCCACCATATTGCAAAGCTATCACAGAAC
CCGTAACAGTAAAAATGCAGTTGCGGAGACTTCTGACCAGGAAGTTAGTGAATCTATGGATTTTAGATATCTGCCAGATGAAAAAAGGTATGAC
ATTTTGCTGGTAATAATTTATATATTTCTTGAAGTGGTCCTGCTAATAACATCTTCTTGTAATATTCATTTGAGTACAGTTATGTATATTCATA
ATTTATGTTTCTTTTCCTGGAAGCTTTCTGTTGGTTTTTTCTTTATGCTCTTTGCATCTCTTTATTTGTTGGGTTGTTTTTTGTTTTGTTTTGT
TTTGTTTTTGAGACAGAGTCTCACTGTCACCCAGGCTAGAGTGCAGTGGCGCAATCTTAGCTCACTGTAACCTCCTCCTCCCGGGTTCAAGCGA
TTCTTCTGCCTCAGCCTCCTGAGTAGCTGGGACTATAGGCACGTGCCACCACGCTGGGCTACTACTTTTGTATTTTTAGTAGAGACAGGGTTTC
ACCATGTTGGCTAGGCTGGTCTCTGCATCTCTTTCTTCAAGAAGAGGTTTGATTAGTATGGAATAATTTCAAAGTATCTAAACTGCTTACTAAA
ATTTCTATTTTATTTTTGAAGGTCTGTTTTAGTCTTTGTGCACTTACCACTCAGAGAGTAGTCTCTATATTGGTTTCTTATCAAACCTTATTGC
ATTAGTTGACATTATTGACTTGTGTGATAAGAAAACAAGATGAATATTTTAAACAGCTTGTTTTGGGTTTTTTTTGAGACAGGGTCTCACTCTG
TCACTCAAGCTGGAGTACAGTGGCATGATCGTGGCTCACCAGAGCCTCAGCCTCCTGGGCTCAGGTGATCATCCCACCTCAGCCTCGTGAGTAC
CTGGGACTATAGGTGCGCACCACCACACTTGGCTAATTTTTGTGGGGTTTTTTTGTTTTTTTGTTTTTTTGTTTTTGAGATGGAGTCTTGCTCT
GTCTTGCCCAGGCTGGAGTACAGTGGCGCAATCCTGGCTCACTGCAGCTTCTGCCTCCTGGGTTCAAGCAATTCTCCTGTCTCAGCCTCCCAGG
TAGCTGGGACTACAGGCATGCGCCACCACACCCGACTCATTTTGTATTTTTAGTAGAGATGGGGTTTCACCATGTTGGCCAGGCTGGTCTCAAA
CTCCTGACCTCAGGTGATTCACCCACCTCAGCCTCCCAAAGTGCTGGGATTGCAGGTATGAGCCACTGCGCCTGGCCAATTTTTCTGTTTTTTT
ATGGAGACAGGGTTTTGTCATGTTGCTTAGGTTGGCCTCAAACTCCTGCGCTCAAGTGATCCACCTGCCTCAGCCTCCCAAAGTGCTGAGATTA
CTTTAAACAATTTTTTACTAAGAGAAAAATATCCTTTTCTACTGATACTACTGATTTGGGACATATTCTATAATACTTATTAAATCCAACTTAC
ATACATATTTATTGAAAACATTTTCTATATGTGAATAAAATATTTGTTTATTAAAGGAGAGGATACAATCCTATAATTTTTTATCTGCTTTCCT
GGTTTCTTTCTAATCAGATACTTACGGCAATAAAGCAAAGAAACAAAAGACAACTCTGCTTTTCCAGAAACTGTGCCAGGATCACGGTAAGAAT
AGTTTGGATCGATTCATATTTAAATAGGTTTTGTTTTATTTACTTTATTCAGTTTTTCAAATTTTATTATTTTTGTTTTGTTTTGTTGTTTGTT
TGTTTTTTGAGATAGCATCTCTCTCTGTCACCCAGGCTGGAGTGCAGTAGGGCAATCTCAACTCACTGCAACCTCTGCCCCCCAGGTTCAACAA
TTCTCCTGCCTCAGCCTCCCGGGTAGCTGAGATTACAGGCATGTGCCACCACACCCGGCTAATTTTTGTATTTTTAGTAGAAACAGGGTTTCGC
CATGTTGACCAGGATGGTCTTGAACTCCTGACATCAGGTGATCCACCCACCTTGGCCTCCCAAAGTGCTGGGATTACAGGTGGGAGCCACCACG
CCCGGCCAGTTTTTCAGATTTTAACTGATAATGTTATTTTTCAAATGACATTTAATAATGGAAAAACTTTATCCTAACAGTTAATTTTCCTGAG
AGACCAAGACCTGGTCTCCTCGGTTCAATTGGAGAAGGAAGATACTTCAAAAAAGGTATTTTATTTCCTATAGCATATTTCTTGTGATCAGAAA
GACCAGTACTTTGCACAATATATTGGAATTCTATTTTTAGGAATGAATAACTGCTTCTTTGAAAATTTTCTCTCCAGATTTTTCATGCTTTCAG
TATAGAATGAATTAAGCATGGCTTCTGAAGGTTGTGTTAGAAACTGATTATGTTTTTGGGATTTATTGTTCAAGAAATTACATGTTGTTCATAT
GGTAGCAATTTTATTAATTACATTGCGTTATGAAAAAATGTTACTGATTTGGTTAGTTACCTGTTTTTATAAATAACATTTTACTGGAACACGG
CCATGAATATTTGATTAAATATTATCTCTAAACTGCTTTTTTTTTTTAACACTATGGCAACAGGGTTGAATAGTTCCAACAGAGGCTGTATATA
GCTCACAAAGCCTAAAATATTTACAATCTGACTTTTTACAGAAAAAAAAATTGCCAATCCCTAGTTTAGGTAATTCAACCGAGGGCTAATTAGA
AGAAGTCTTTTTTCCCCCACGTACATTCTTAAATCATTGCTTTTTCCTGGTTTAGTTTGTAAAGCATTTGTATAATATGTAAATAGATACAGAA
TTAATTTTTTCTGTAATTTTTTTCTGACTGTGAATTTTATAGTCTCTTAGGGTTCTGTTGTAATATATCTTAAAAGTTATGTGTATTTTCTTAT
CCCTAATGAGTGTTGTCTCCATCCTTCCATTTTCTCCTAAAAGTGAAGAGTAAGAGATGATTTTGGCTATTTGGAATCCCATTAATTGTAATTC
TAATATTTATCTTTTAGTTTTCAAAAATACCTTTATAGAGCCTCAGAAACTAGATTTTCATATTTAATTGAAGTGAAATTACATATGCCAAAGT
AACTTGCCAAAATCATTTTACCTGTATTTGTAAAATGGTATATACGTTACAGAATTTCATTTTGGATTCGTGTAATAATTTTAGTGCTTTTTAC
ATTTTTTTATTTGAAATGTTACATTGGTTTCCTTGACATTTTTCTTTATATATATTTGTGTGCTTATGCAATTTTAATTTAGAAATGCTTTTTA
TAATTTTGTTCATTTTAATTTCGTAAAATAAATTTTTCCTCCCACAGAACCAAACTTGTTTTCTCATGATGCAGTTGTGAGAGAAATGCCTACA
GGGGTTTCAAGTCAAGCAGAATCCTACTATCCCTCACCTGGGCCCATCTCAAGTGGATTGTCACATCATGCCTCAATGGCACCTCTGCCTTCTT
CAAGCTGGTCATCAGTGGCCCACCCCACCCCACGCTCAGGCAATACAAACCCACTGAGTAGTTTTTCAACAAGGACACTTCCTTCTAATTCGCA
AGGTATCCCACCATTCCTGAGAATACCTGTTGGGAATGATTTAAATGCTTCTAATGCTTGCATTTACAACAATGCCGATGACATAGTCGGAATG
GAAGCGTCATCCATGCCATCAGCAGATTTATATGGTATTTCTGATCCCAACATGCTGTCTAATTGTTCTGTGAATATGATGACAACCAGCAGTG
ACAGCATGGGAGAGACTGATAATCCAAGACTTCTGAGCATGAATCTTGAAAACCCCTCATGTAATTCAGTGTTAGACCCAAGAGACTTGAGACA
GCTCCATCAGATGTCCTCTTCCAGTATGTCAGCAGGCGCCAATTCCAATACTACTGTTTTTGTTTCACAATCAGATGCATTTGAGGGATCTGAC
TTCAGTTGTGCAGATAACAGCATGATAAATGAGTCGGGACCATCAAACAGTACTAATCCAAACAGTCATGGTTTTGTTCAAGATAGTCAGTATT
CAGGTATTGGCAGTATGCAAAATGAGCAATTGAGTGACTCCTTTCCATATGAATTTTTTCAAGTATAACTTGCAAGATTTAAATCCTTTTAAAT
CTTGATACCACCTATATAGATGCAGCATTTTGTATTTGTCTAACTGGGGATATAATACTATATTTATACTGTATATATAATACTGACTGAGAAT
ATAATACTGTATTTGAGAATATAAAAAACTTTTTTCAGGGAAGAAGCATACAACTTTGGACATAGCGAATACAAAATTGGAAGCTGTCATAAAA
AGACAACTCAGAGGCCAGGCGCAGGGGCTCACACCTGTAATCCTAGCACTTTGGGAGGCCAAGGCGGGTGGATCACTTGAGACCAGGAATTCGA
GACCAGCCTGGCCAACATGGTGAAACCCCGTCTCTACTAAAAATACAAAAATTAGCTGAGCATGGTGGTACGTGCCTGTACTGTCAGCTACTTG
GGAGGCTGAGGCACAATAATTGTTTGAACCCAGGAAGCAGAGGTTGCAGTGAGCTGAGATCACACCACCGCACTCCAGCCTGGGTGACAGAGTG
AGACTCTGTCTCAAAAAAAAAAAAACAAAAAAAACACACTTTTTTATATTTCTTTTTATAATGTTTTAATGTATTCTTAAATTTCAAGCAAATT
TAAGATAAAACTTGTAATGGCTATGCCATTGAAAAACTTAATTTTTTATTTTTGAGGCCCATGGGCCAAGGTAACCCCTAAGGGGTTTTCTTAG
GCTTCTTGGAGCTTAGATTTGTATGTATATCAAAATGTCTTTAAAATGTTAAGTTGGGCAGAAGGCAGTTGAAGTGAGCTTTCAAGGTATGGGA
GGTTTTCTACATTTTATACTATTTCAATCTATGCCTTTAAAGTTGCTTATGATTTTAGCTGTACACTCATTTTTTAAGGGGAAGAAGTTTCCTT
GGACCATTCGCCTTTCTTAGATGTCCTCACTCCCTGTGATCTCATAAAACTGCCTATTTGACATCTCTATCTAGAAATCTAATTAAAGCTCACA
CTCAGCATATCCAAAACTGAATTCTTGGTCTTCCCTCCCAAACTTGCTTCTCCTTCAGTCTTCTCCAACTCAGTAAATGGCAATGCCATACTTC
TGGTTGCTCAGGCCAAAAACCCTGAAGTCATCCTTGATTCTTCTTTTAGCACCCATATCCAATCCATTAGCAAATCTGGTAGACCCTACCTTCA
CAATATATCTAAAGCTGACCACCTCTTTTGACCTCTACTATTAACGCCCTATTCCAAGCCACCATCATCTCTTCCCTGGATTGAAGCTGTCATC
TACAAAAATTCTTCTTATATCCTTGCATTCCTACAGTCTGTTCCCCATAAAGTAGCCAGAATGATTATTTTTAAAACAAGTCAACTCATACCAT
TCATCTGCTCAAAACCATTCATTGGCTTCTCATCTCACTCAGAGCAGTCAAAGTCCTTAAAAGTTGCAGGCCTAGACTCCCTGTCCTACCTCAG
GTACCACCATATCCCACCTCCTCATGCAGCTCCAGGCACCTTGGCCTCAGTGCTCCTCAAAGCATCAAGTATGCACTTGCCTTGGACAGTCTGT
ACTTGGTATTCCCTCTGCCTTGAATGTTGTTCTCCCAGAAAAATGCATAGTTCACTCTTACATCCTTCAGGTGTCACTCCACTGTTACCTGAGC
AGGTCGTCCTTGAATATATACATCAGCATTCCCTTTCCCCCCTGCTTTATGTATGTCCATAGCACTCACCACGATCTGACTTTACTAAGTATTT
ATTCATTTACTGTTTGTTTTCCCATACCGAAATATAAACTTTCTAAGGACAGAAATTTTTGTGCTTTATTGTTGAATCTCCAATTTGTAGAAAA
ATGCCTACCTTATATTAAACACTCAGTAAATGTTTATTGAACATTAAAAGTATTACTAATAGAACTTTGGTTTTTGAAAGAAATAATAACTTTA
ATTATAAGACGTATATGATTTTTGCAGTTTTACTTAGTGTGACATTGGGTTTATGAGAATCGTGTACATTCAAGTCCAGGAATAATAATGGTCA
TCCAAATTGTTTGAAAGGAAAATAATCCCAGTGGCAAAATGATGGTAGAATTTGGGTAATCTTTTTTTTCCTTTTATGAAAAGAGATTTTATTG
AAGGTAAAACATTAGAGGTTCATTGAGAATCTCTAAATCCATGTTTTGACATTGTCAAGCTCATTGCAACTTCCAGATTGAGTAACACTTATAA
CACATTTCCTTTTCAAAGTGCAAGATTTTTAAAAGAGACTTGTCACATATTCATTTGGCTGGTTTCAAATGGTGAGCTGAATGCTGGGTAATCT
CTACTAGCTCCTTAATCAGATTTAAAATTCTCAGTGTTTCCTAGTTGTTTCTGCATACTTTATGTGAGTTGTTATAGCTGTAACATTACACTTT
ATTTGCTGTTTGTGTTTCGTGACTTTTGGTAATTCTGGCATTTAGAAACCTTTCACTTTGCTTCAAAACGTAGTTATATTTTGGAGTTTTCATT
TGATATATAATTATTTATTTTGCCCTTTTATTTCCCAAAGACATTGTAAGGGTTAATTAGATCATTATATTTTATTATTACAGATTAAAGTTGG
GCAGTAATCTTAATTATGATGGAATTATCATTATGCTAAGTAATTAACTTTACCTAGTTTGTTTTACAACTAGAACCTGCCCTAAATGTTGAAT
ATCTTCCTAGCAAGAAACAGTCTGTCATTTTACTTACACGATGTCTAACCAAACCATAACTTTACATAAACTAGTCGTTTCGGTCAAATAGAAA
AATGTGTGAATGCCATAAAAACAAAAATTCTCAGTTAAATGATACTGGGAAATAGGGAAGACAGCAAAGTGAGACTTGGGCTCAGGATGGTTCA
GGAAGAAAAAAAAAGAAAGACCCCCTGAGTACCATTAATATTCCTCAGAAATTATTATTTCAAAAGGAAATATTTCTGTATTATAAATTTTCAT
GAGCAGCCATTATGAAATCTCACAAGAATCATAGAATTCAATAAAAAAAGGTAGAAAGTAATTTTTTTACTTAAAAATATAAATTAAATAAATT
TTTAAAATCATAAGCACATAAATAGAACTTACCAGGGAGAAAGAAAAACCTGAAGGCACAATTTCTTTTCTGTTCAAAATGTGAACCCAGGATG
TCTCTAGATGATGATGGATGATAGGTGGGGAGATTTTTTTTTTTTTTAATACAGAATCTCATAGTTTTGGATTAATTAGCACCAATCAGTTTAA
ACACTGACTGTTAGAATAGCTGCATGGGTTTTTTTCTTTAAACTAATTAAGCGTTGGCTACTTAGTATAAGTAAGTATAAGCCGAATTAAGGTT
CTGCTACATCTGTGTTTAGAATATTTTTTTAAAACTAAATAAGTGTTGGCTAGTTTTGCGGTGTAAGCAGAATTAAGGTTCTGCTACCTCTGTG
TTTAGAATATTCCCAATGGATTTTTCATTTTTCAGGTGCTATTTTTTGACCCTGTATAGACTTTAATTTAAAATGAATTTGGTAACGTTTCTCC
TCTGTCTCTACATATATTCATGCTTTCACCTGCTCTTTTAACACCTGCTTTTAGTATCTGAGGCACTTTTTCTGAACTCTACTTGTGCACTGGA
TCCCTCCTCCTTTCTCTGCCAGGCTGTGTTTACTTTATCCTTACATCACCACTTAGTGATTCCTTTCTTTGTATAAACATGGTAAATGTCTTCA
TTAGCCTAAAAGGAAAGACCAAATAAAACCTTTCCTACCACTTGGATGCATTTGCATCCTGACTTCTGAAATGCCTCCAGCCTCCATTTTCTCC
CTTCCCAGTTATTCCTTAGCCCAGCCATCTCTGTCTTTAGCTCCTACAATTTTCTTAGGATATTCTGGGAAAGATGAGCGGAGACTGCCCGCCT
TGTCAAATCTAGTGTCTTTTTTTCAGTCCTCACACTGCTTGACCTATGTATAACCTCCTATACTTCCCTCTTTGCATACTCCTCTGGGTTTTCT
GTGGTAGTCAAGATTCCTCCCTGAGATTTATTTCCCATGAGTCTTGACCCCTCCCCTCAGTTGGTGCTATTTCCCCCTACCCGCCCTCCGATGA
TCTTATCAGAGCCCACAGGTTCAGTTTTCTTTCATGCTACCTGAATGTCCTGATAAACTGGCTCGCTCTCTTCTTTACCTTCCATAATGGCATT
ACCATTTACCACGCCACCCAAGATACTTACTAGGAACCTCAAAGTATTGTATTCTTTTTCTCCATCACACTCATACTTAATCATCAAGTCCTTT
TGAGCTTGTCTCCTCTTGAATATGTCCCTTCTTAATTCCTGCTGCCTTCTTAGTAAAGGCCTTCATTCTTTTTTCCCTAGTAATAATCTTTTCC
ATATGTTCCAGTTAAAATACCATGTTCTCCCTATTCCTTATTACATAGCTAGCATTCCTTGAAAAAAAACAATTCTCTCAGGCCTCCATACCTT
TAGCATGTTACCCACTCTGCCTCTGCTCTTCTGGAACTAGAACACTCATCCTTGAAGGCTGGGCTTCTGTATGAAGGTTGGTCCTGCCTCCTTA
CTTGAGGTGAAGCTTTGTACATGCCTGTATTACGGACATCCTCTTATTTAAGTGTTTGTCTCTTTCGTCATTGGGACTCCAGCACCCAGCATAG
TCCCTAGTATACTAGTTGGTGCCGAATAAATAGTAGCTATTATTAGAAAAGGAAGGGTGAAATTGACATGGGAGTTAGTAAAATGTATATGGAA
ATGATTTTTAAAGGGAAAGGTAATGATTTTCTGGCAGGAAAAGCAGCAATGACAAGATTACTTAAGTCTTGTGAAATAACACTTCTCTTCCTTG
ACCTGCTGCTTCCCTTTTTTACCACACACACACGCACACATACCACAGCCCTTTGAGACTGAAAGCAGCTCTATTGAGAATAGTAGTGTCAACT
GTATTATGTAGAAATTCTAAAGTTTTTGGGATTATTTCATAGCCCTGACCTTGCTACTTCTCTCCACTTTATGTGGCAGGTTTAATCTCAGGTC
TCCCTCATACACTTCTCAGCCTCAGCACCTAACCCTCACACAACACTCCAGTATTGATGCAGTCAATCTTGTATAACATTTTTTGAATGTCCAA
TGTGCAAAGCACGATGTTGGAAATTATACAGAGGTGAATAAGACAAAAACTCTTGCTCTCAAAGATGTCAGTCTTTTTCTTTGCAAGGATAACA
GACATTTCCAGCACCCCTGAAATTTCCTTCATGCCCCTTCCCAGTCTGTACCTACACCTCTAAATCTATTTTCAATCTTAATGGCCTTTTAAAT
AACTGGGCTTCTCACAACCATAGTGAACAGAAACAGCTGGGTTGTCAACCTCTAACCTAATACTTCAGGAAAACTCATGATGGTTTCCATGTTA
AGAGAGACATGGAGCAGGGCACTGGCATGGTGGATGGATCACGCCTGTAATCCCAGCACTTTGGGAGGCCGAGGTAGGGGGATTGCTTGAGCCC
AGGAGTTCAAGACTAGCCTGGGTAATATAAGGAAAACCTGTCTCTGCAAAAAAAAAAAAAAAAAGAGGATACAACCAAATGGAAGAACATTCCA
TGCTCATGGGTAGGAAGAATCAATATCGTGAAAATGGCCATACTGCCCAAGGTAATTTACAGATTCAGTGCCATCCCCATCAAGCTACCAATGC
CTTTCTTCACAGAATTGGAAAAAACTACTTTAAAGTTCATATGGAACCAAAAAAGAGCCCATATCGCCAAGTCAATCCTAAGCCAAAAGAACAA
AGCTGGAGGCATCACACTACCTGACTTCAAACTATACTACAAGGCTACAGTAACCAAAACAGCATGGTACTGGTACCAAAACAGAGATATAGAT
CACTGGAACAGAACAGAGCCCTCAGAAATAACGCCGCATATCTACAACTATCTGATCTTTGACAAACCTGAGAAAAACAAGCAATGGGGAAAGG
ATTCCCTATTTAATAAATGGTGCTGGGAAAACTGGCTAGCCATATGTAGAAAGCTGAAACTGGATCCCTTCCTTACACCTTATACAAAAATCAA
TTCAAGATGGATTAAAGACTTAAACGTTAGACCTAAAACCATAAAAACCCTAGAAGAAAACCTAGGCATTACCATTCAGGACATAGGCATGGGC
AAGGACTTCATGTCTAAAACACCAAAAGCAATGGCAACAAAAGCCAAAATTGACAAATGGGATCTAATTAAACTAAAGAGCTGCTGCACAGCAA
AAGAAACTACCATCAGAGTGAACAGGCAACCTACAAAATGGGAGAAAATTTTCGCAACCTACTTATCTGACAAAGGGCTAATATCCAGAATCTA
CAATGAACTCAACAAATTTACAAGAAAAAAAACAACCCCATCAAAAAGTGGGCGAAGGACATGAACAGACACTTCTCAAAAGAAGACATTTATG
CAGCCAAAAAACACATGAAAAAATGCTCATCATGACTGGCCATCAGAGAAATGCAAATCAAAACCACAATGAGATACCATCTCACACCAGTTAG
AATGGCAATCATTAAAAAGTCAGGAGACAACAGGTGCTGGAGAGGATGTGGAGAAATAGGAACACTTTTACACTGTTGGTGGGACTGTAAACTA
GTTCAACCATTGTGGAAGTCAGTGTGGCGATTCCTCAGGGATGTAGAACTGGAAATACCGTTTGACCCAGCCATCCCATTACTGGGTATATACC
CAAAGGACTATAAATCATGCTGCTATAAAGACACATGCACACGTATGTTTATTGCGGCATTATTCACAATAGCAAAGACTTGGAACCAACCCAA
ATGTCCAACAATGATAGACTGGATTAAGAAAATGTGGCACATATACACCATGGAATACTATGCAGCCATAAAAAATGATGAATTCATGTCCTTT
GTAGGGACATGGATGAAATTGGAAAACATCATTCTCAGTAAACTATCGCAAGAACAAGAAACCAAACACCACATATTCTCACTCATAGGTGGGA
ATTGAACAATGAGAACACATGGACACAGGAAGGGGAATATCACACTGGGGACTGTTGTGGGGTGGGGGGAGGGGGGAGGGATAGCATTGGGAGA
TATACCTAATGCTAGATGACGAGTTAGTGGGTGCAGTGCACCAGCATGGCACATGTATACATATGTAAGTAACCTGCACAATGTGCACATGTAC
CCTAAAGCTTAAAGTATAATAAAAAATAAATAAATAAATAAATAAGAAAAAGAAAGCCAGGCATGGTGACATGTGCCTGTGGTCCCAGCTATTA
GGGAGGCTGAGGTGGGAGGATCCCTTGAACCCCAGGAGGTTGAGTCTGTAGTGAGCAGTGATTACGCCACTGCACTCCAGCCTGGGCAAGACCT
GTCTCAAAAAAAAAAAAGACTTAGAATTGGTGATCCAGGCCGCCTAATGGCATCAAATAATTTGTTATATCTTTAATTTATTGAAGGATCACCA
CATGCTTTTAAATAGCATGGAGAAATGGAAAGAATAGGGACTTTTTACTCAGGTAATACCCAGCCTGCTACCTAACAGGTTGTGTTGTACTAAA
TAAAATGGTACATAAGAAGAAACACTGTAAATTATATAGTGCGAATCAAATATTGTTAATAAACCAATATCTGTATATCCTATGTCCCGGATTA
ATCTTTAATTTAGATACTCCTTCTAGTTATCTAATACACAGCAGAGTGAGAAAAATCATTATGGATTAGGTTCTTTAGTAAGAAACCTGAGATG
GACTTCTCATTAGCATTAACTAGTTATTGCCCAGCTTTGGAGAGCCTTCTTTTGGCTTATCATTTATTATAAGCCCAGAAATAGGTGACTAATC
AGAGATAAATGTATGGGTTGTCTGTATCTAGTTTTATGCCTTTTTTTTGCCTAAGACGTAGTCAAATTAATATTTTAACTAATACATCTTAGCA
GAGTTTAGTTAAGCACAAAGTTAACAGTGGGTAGGATTGAATCTTGAAAGTAATCATGTCTGTAATGTTTTTCATGCATGCAAAAAGCACAGAC
AAAACCACTCATGCCCTATTAATAAACAAAATACAGATCAAAGTTTTCAAAAGTAATCACTCTATTTATTCTAAATGTCTGTGGCTTTAGGAAA
ATACCACCAGCTAGTACTTACCTATTTAAAGATGTAGAATTTATTATCCTCTAATATTCTTATCAGTTGTTTCCACAACTTTAGTTTACTATTG
GACTTTCAAAAATTTAAAGAATTACAAGTAAAATTCATTAAACACTTGTGTGTGAATAGTAATACACAGTAATTAGTACAGCATGTTGCTTCTT
CAACAAATTGAGTTTTCAGGGAAATCAGCAAGTAAATGAAATATAAATTTTTGGTAAAAGTATCAAACATTCATCTTGCCCATTTTTCCTCTTA
AACTTTATTATCTAATCAAACATAGTTTTCCATAAGATGTAATAAAATATAGATAAGGTTGGAATATTTGAGGATCCATTTGTGGAACTGAATT
TAATGAGACTTCATTGGTGATACACTCAATTTTTACTGGGTAATTAGCTAATAATGTTGGTCACTGTCTCACAGTTCAAGTAGCTTTAAGATGA
TGTGGCAAGGAAAACACAAAGCTTTTGGGTAACCAGCGTTCTTAAATGTATGGTTTTTGACCAGGTGAACCCTTTAGAAGTGATTTCTGTTTTA
AAAGTATGTACTTAAAATACCTTTGGCTGTGATGAATGTAGATCCCAGCAGAATACCAAAATCCTATTTTTTTTGACTGAGTATTTGTAGATGC
TTAATGACTGAAATGAATTTGGAGGCACTGATGAAAGTGATTTTTTTAAAGTTCTCAGGTACTGTTCAATTATTTAATGTTAAGTTTAGTATCA
AGATACAGTTGTTTTTAAAATGCCAAAATGCTGTTTATTATACAGAATATTTTATTACATTTGCAATATCTTTGTATATAGTGATTTTTTTCTT
GATAATAAATGGAAAAATTCTAAAACACTTGCTGAAATTTTGTTGCTATTTAACTTTTAAAATATCAGATTTTCTTCATATTTATAGCATCTTC
CTAACAGGAAAAGGTAGTTTTAGTTGCTTAGAAACATTAGACATTTTTATGGAATGCTGTTGCTGAATATGGTTGCATTTGTAACTGTCCTTTG
AAATAATTTTGTAGAGTACTGAAGTGCCTTTTTACAAGATTTTTTTCCTTTTTCCTTTTCTGGCTTAACCAACAAAAATCTGTTTTCAGAGTCC
TGGAGGCTGAAAGTCCGAGATCAGGGGTACTAGGACGGTTTCTGCTGAGGCTCTCCTCCTGGCTTGCAGAGGGCTGCCTTCCCATCATCCTCAC
ATGGTAGAGAGAGAGAAGGATGGAGATCTAGGGTGTCTCTTCTCTTTTTTGAGACAGAGTCTCACTCTGTCATCAAGGCTGGAGTGCAGTGGCA
CAATCACATCTCACTGCAACTTCTGCCTCCTGGGCTCAAGAGATCCTCCCGCCTCAGCCTCCCAAGTAGCTGGGACTACAGGCGCATGCCACCA
TGCCTGGCCAATTTTTGTATTTTTTGTCAGAGTTTCATCATGTTACCCAGGCTGGTCTCAAACTCTTGGGCTCAAGTGATTCACCCGCCTTGGC
CTCCCAAAGTGCTGGGATTACAAGCGTGAGTCACCATAGCTGGCCTGGCCTCCTCTTTTCTTAAATCCTTTTTTTTTTAATTGACAAAACTGTA
TATATTAATATTTATCTTGTTCAACATGATGTTTTGAAATATGCATACGTTGTGGAATGGCTAAACTGAGCTAATGAACATGTATTACTTCACT
TAGTTATTTTGTAAGAACACTTAAGATCTACTCTTAGCAACTTTGAAGAATATATTTGTTATTAACTATAGTCACCATGTTGTACAGTAGATCT
CTTAAACTTATTCCTATCAAGCTGAAATTTTAAATCCTTTGACCAACATCTCCCCAACTCCGCAACCCCTATCCCACTCTCACCTCCGCAGTTC
TAGTAACAACCATTCTACCCTACTTCTATGAGTTCAACTATTTTAGATTCCACATATAGGTGAGATCATGCAGTAGTTGTCTTTCTGTGCCTGA
CTTATTTCACTTAACATAATGTTCTTCAGGTTCATCCACGTTGTTTCAAATGACAGAATTTCCTTTCTTATGGGTGAATAATATTTCATTGTAC
ATATACACCACATTTTCTTCATTTATCT
HUMAN SEQUENCE - mRNA
CGGAAGGTGTGAGCCGCAAACCCAGCGGAGGGCGGGAAGAAGGAGGAGGCCTCTAGGGTGNTCGGGGGACTGGGGGCCCCGCCGGCAGAGGTCC
CTCGGCCTCCTGACTGACTGACTGCGGCCGCCTCCGGCCAGGACGCTGGGAGCTGCCTGCGGGAAGGTGCGGGGAGCGGAGCCATGGCCTCCGG
TGCGTATAACCCGTATATAGAGATAATTGAACAACCCAGGCAGAGGGGAATGCGTTTTAGATACAAATGTGAAGGGCGATCAGCAGGCAGCATT
CCAGGGGAGCACAGCACAGACAACAACCGAACATACCCTTCTATCCAGATTATGAACTATTATGGAAAAGGAAAAGTGAGAATTACATTAGTAA
CCAGGGGAGCACAGCACAGACAACAACCGAACATACCCTTCTATCCAGATTATGAACTATTATGGAAAAGGAAAAGTGAGAATTACATTAGTAA
CAAAGAATGACCCATATAAACCTCATCCTCATGATTTAGTTGGAAAAGACTGCAGAGACGGCTACTATGAAGCAGAATTTGGACAAGAACGCAG
ACCTTTGTTTTTCCAAAATTTGGGTATTCGATGTGTGAAGAAAAAAGAAGTAAAAGAAGCTATTATTACAAGAATAAAGGCAGGAATCAATCCA
TTCAATGTCCCTGAAAAACAGCTGAATGATATTGAAGATTGTGACCTCAATGTGGTGAGACTGTGTTTTCAAGTTTTTCTCCCTGATGAACATG
GTAATTTGACGACTGCTCTTCCTCCTGTTGTCTCGAACCCAATTTATGACAACCGTGCTCCAAATACTGCAGAATTAAGGATTTGTCGTGTAAA
CAAGAATTGTGGAAGTGTCAGAGGAGGAGATGAAATATTTCTACTTTGTGACAAAGTTCAGAAAGATGACATAGAAGTTCGTTTTGTGTTGAAC
GATTGGGAAGCAAAAGGCATCTTTTCACAAGCTGATGTACACCGTCAAGTAGCCATTGTTTTCAAAACTCCACCATATTGCAAAGCTATCACAG
AACCCGTAACAGTAAAAATGCAGTTGCGGAGACCTTCTGACCAGGAAGTTAGTGAATCTATGGATTTTAGATATCTGCCAGATGAAAAAGATAC
TTACGGCAATAAAGCAAAGAAACAAAAGACAACTCTGCTTTTCCAGAAACTGTGCCAGGATCACGTAGAAACAGGGTTTCGCCATGTTGACCAG
GATGGTCTTGAACTCCTGACATCAGGTGATCCACCCACCTTGGCCTCCCAAAGTGCTGGGATTACAGTTAATTTTCCTGAGAGACCAAGACCTG
GTCTCCTCGGTTCAATTGGAGAAGGAAGATACTTCAAAAAAGAACCAAACTTGTTTTCTCATGATGCAGTTGTGAGAGAAATGCCTACAGGGGT
TTCAAGTCAAGCAGAATCCTACTATCCCTCACCTGGGCCCATCTCAAGTGGATTGTCACATCATGCCTCAATGGCACCTCTGCCTTCTTCAAGC
TGGTCATCAGTGGCCCACCCCACCCCACGTCAGGCAATACAAACCCATGAGTTAGTTTTTTCAACAAGGACACTTCCTTCTAATTCGCAAGGTA
TCCCACCATTCCTGAGAATACCTGTTGGGAATGATTTAAATGCTTCTAATGCTTGCATTTACAACAATGCCGATGACATAGTCGGAATGGAAGC
GTCATCCATGCCATCAGCAGATTTATATGGTATTTCTGATCCCAACATGCTGTCTAATTGTTCTGTGAATATGATGACAACCAGCAGTGACAGC
ATGGGAGAGACTGATAATCCAAGACTTCTGAGCATGAATCTTGAAAACCCCTCATGTAATTCAGTGTTAGACCCAAGAGACTTGAGACAGCTCC
ATCAGATGTCCTCTTCCAGTATGTCAGCAGGCGCCCAATTCCAATACTACTGTTTTGTTTCACAATCAGATGCATTTGAGGGATCTGACTTCAG
TTGTGCAGATAACAGCATGATAAATGAGTCGGGACCATCAAACAGTACTAATCCAAACAGTCATGGTTTTGTTCAAGATAGTCAGTATTCAGGT
ATTGGCAGTATGCAAAATGAGCAATTGAGTGACTCCTTTCCATATGAATTTTTTCAAGTATAACTTGCAAGATTTAAATCCTTTTAAATCTTGA
TACCACCTATATAGATGCAGCATTTTGTATTTGTCTAACTGGGGATATAATACTATATTTATACTGTATATATAATACTGACTGAGAATATAAT
ACTGTATTTGAGAATATAAAAAACTTTTTTCAGGGAAGAAGCATACAACTTTGGACATAGCGAATACAAAATTGGAAGCTGTCATAAAAAGACA
ACTCAGAGGCCAGGCGCAGGNGCTCACACCTGTAATCCTAGCACTTTGGGAGGCCAAGGCGGGTGGATCACTTGAGACCAG
HUMAN SEQUENCE - CODING
ATGGCCTCCGGTGCGTATAACCCGTATATAGAGATAATTGAACAACCCAGGCAGAGGGGAATGCGTTTTAGATACAAATGTGAAGGGCGATCAG
CAGGCAGCATTCCAGGGGAGCACAGCACAGACAACAACCGAACATACCCTTCTATCCAGATTATGAACTATTATGGAAAAGGAAAAGTGAGAAT
TACATTAGTAACAAAGAATGACCCATATAAACCTCATCCTCATGATTTAGTTGGAAAAGACTGCAGAGACGGCTACTATGAAGCAGAATTTGGA
CAAGAACGCAGACCTTTGTTTTTCCAAAATTTGGGTATTCGATGTGTGAAGAAAAAAGAAGTAAAAGAAGCTATTATTACAAGAATAAAGGCAG
GAATCAATCCATTCAATGTCCCTGAAAAACAGCTGAATGATATTGAAGATTGTGACCTCAATGTGGTGAGACTGTGTTTTCAAGTTTTTCTCCC
TGATGAACATGGTAATTTGACGACTGCTCTTCCTCCTGTTGTCTCGAACCCAATTTATGACAACCGTGCTCCAAATACTGCAGAATTAAGGATT
TGTCGTGTAAACAAGAATTGTGGAAGTGTCAGAGGAGGAGATGAAATATTTCTACTTTGTGACAAAGTTCAGAAAGATGACATAGAAGTTCGTT
TTGTGTTGAACGATTGGGAAGCAAAAGGCATCTTTTCACAAGCTGATGTACACCGTCAAGTAGCCATTGTTTTCAAAACTCCACCATATTGCAA
AGCTATCACAGAACCCGTAACAGTAAAAATGCAGTTGCGGAGACCTTCTGACCAGGAAGTTAGTGAATCTATGGATTTTAGATATCTGCCAGAT
GAAAAAGATACTTACGGCAATAAAGCAAAGAAACAAAAGACAACTCTGCTTTTCCAGAAACTGTGCCAGGATCACGTAGAAACAGGGTTTCGCC
ATGTTGACCAGGATGGTCTTGAACTCCTGACATCAGGTGATCCACCCACCTTGGCCTCCCAAAGTGCTGGGATTACAGTTAATTTTCCTGAGAG
ACCAAGACCTGGTCTCCTCGGTTCAATTGGAGAAGGAAGATACTTCAAAAAAGAACCAAACTTGTTTTCTCATGATGCAGTTGTGAGAGAAATG
CCTACAGGGGTTTCAAGTCAAGCAGAATCCTACTATCCCTCACCTGGGCCCATCTCAAGTGGATTGTCACATCATGCCTCAATGGCACCTCTGC
CTTCTTCAAGCTGGTCATCAGTGGCCCACCCCACCCCACGCTCAGGCAATACAAACCCACTGAGTAGTTTTTCAACAAGGACACTTCCTTCTAA
TTCGCAAGGTATCCCACCATTCCTGAGAATACCTGTTGGGAATGATTTAAATGCTTCTAATGCTTGCATTTACAACAATGCCGATGACATAGTC
GGAATGGAAGCGTCATCCATGCCATCAGCAGATTTATATGGTATTTCTGATCCCAACATGCTGTCTAATTGTTCTGTGAATATGATGACAACCA
GCAGTGACAGCATGGGAGAGACTGATAATCCAAGACTTCTGAGCATGAATCTTGAAAACCCCTCATGTAATTCAGTGTTAGACCCAAGAGACTT
GAGACAGCTCCATCAGATGTCCTCTTCCAGTATGTCAGCAGGCGCCAATTCCAATACTACTGTTTTTGTTTCACAATCAGATGCATTTGAGGGA
TCTGACTTCAGTTGTGCAGATAACAGCATGATAAATGAGTCGGGACCATCAAACAGTACTAATCCAAACAGTCATGGTTTTGTTCAAGATAGTC
AGTATTCAGGTATTGGCAGTATGCAAAATGAGCAATTGAGTGACTCCTTTCCATATGAATTTTTTCAAGTATAA

TABLE 16

MOUSE NOMENCLATURE

| | |
|---|---|
| ICSGNM | Icsbp |
| Celera | mCG21199 |

HUMAN NOMENCLATURE

| | |
|---|---|
| HGNC | ICSBP1 |
| Celera | hCG17046 |

MOUSE SEQUENCE - GENOMIC
GCAGGAGGATGGAAAGTTGGAGGGTAGCCTGACTGTATAACAAGTTCAAGGCCAGCCTGAGATACATAGTGAAATCCTGTCTCAAATACAAAAC
AAAACAAAACAAAACAAAACAAAACAAAACAAAACAAAAAAAAAAAAAACAAAAACCCACCCCAGGCGTGATGGTGCATGCCTTTAATCTCAGC
ACTCAGGAGGTAGAGGCTGGAGAGTCTTTGTGAGTTCAAGGCCAGGCTGCTCTACAAAGAGAGCTCCAGGACAGCCAGGGGTACCTAGAGAGAC
CCTGACCAAAACAAAACAAAACAAAACAATCAAAAAACAACTTAACAATCCTTCCAACTCTCCAAACCCAACGCCACCCCCAAATCAACATCAA
ACTCTCTCAAGTCCCGGAAAGCAACCAAACAGACAACACCTAACCAAGCTGAGAAGCGGACAGCACTGGAGCCTTCTGAAGCACTAACCTCTTT
TAAAGAGGGGGCGCAGCCCAATTCACCAGTGAGCAGATTGGCTTAGAAAGGGGCAAGCGCTCCTTGTGGGACACAGTCACCACTTGTTGATGAG
GTTGCTTGCAGGGTTGACGCCATGGCTCAGCAGGTAGAGTCACTTGCCACCAAGCCTGACACCTGATCTGCATCCCAGGAGCCACAGGGTGAAA
AGAAAAAGCAACCCTCTGGCTTCCACACAGGCACCATGGCATGGGCTCCCTGCTAAACACGTATGCGTGCGCACACGCACATGCATATGCATGC
ATGCACATTCATATGCACACACATAGCGCACACATACACAAAATTATTAATTAAAATGTAATTTAGATTCGGAGTGGACACGGCGGTCGCCGCC
TCTAAAGAGAAGACTCACCACATCTATGGCTGGGGTTCTGAGCTCAGTGTTTCAGAGGCCTCCAGGCAGACTGCAGACTGTGAAGGAAGGTGCA
GAATCTCTCATTGGTACAGAATGGATTCGTCACACATTTACCAGTTCAAGAATTCCCGATAAAGTGTTTCGGCCTAGACCTGAAGATCATGAAA
AGTACAGCGGGGACCCCCAGAGGCCTCATAGACTGTACATTGTCATAAGAATAGGAAGTACAAAGAGGCATCCAGGGCTGGAGAGATGGCTCAG
CGGGTAAGAGCATTGACTGCTCTTCCGAAGGTCCTGAGTTCAAATTCCAGCAACCACATGGTGGTTCACAACCATCTGTAATGAGAGCTGACTC
CCTCCTCTGGAGTGTCTGAAGACAGCTACAGTGCACTTACATATAATAAATAAATAATTCTTAAAAAAATAAAAGAGGCGTCCATACTGGGAAA
AAGATACGATCAAGATGCTCGGACTACAGAAGGCACACAGCCCTCAAATTCACAAGAACATCCCACCGGTGGATGCAAAGCTGAAAGTGGTTAA
ACCCTAGATAGGCATCCAGCGCCTGGAGCTGCCACAGGGACTTCCCACAGAAAAGACCATGTCCAGCGCGTGCCTCAGGGGCACCGGGGAGTTG
GTCATGTGGTGGCATCTGAAGCCTGTGGAGCTGGAAGTGACGTCCTGACGCCACACAGCTTCAGCTCACAGACACACTTACTGAGGAAGCCGTT
CTCATTAAAGTGTATGCTCATCAAAAAAAAAAAAAAAGTAACTTAATTTAAAAAAGGATTTCTTGTAAGCGATGTCAGTAATCTGGTGAGTTGG
TGACTCTGTTTCTGGGTACCCTATCTGCTGTTGTACTAAGTTTTTCCTGTGCCTAAGCCGTCTTCCTATTAGAGATGTAGACTTGCTGTGTGTC
TGGTCACTTGTACACAGGTGGGGATACATCGTTCTGAATTCATTGAAATTTCTGAGTGTGTGGCTCCCCTGGCCCTGCCCGAAATATGTTAGGT
ACATCTTCCTGGGGGTTTGTGTGTGTCATAAGCACACTGGTATGAGTCATGTGTGGTTTATATTAAACACACAAACGTGTATTCTTTACAGGGT
CTCATGTAGCCCAGGCTGGCCTCTAACTCATTTAGTTCAGGATAACCTGAATCCCAGAGCCTTCTGTCTCCGCCTCCCAGGTGCTGGGTTACAG
GTGTGTGCCAACAAACCCAGGGAAATCTACGTTTTCAGTAAATTGATATCTCTTGGCAATAGTTCCCAAGAGAGTTCAAAGACATGTTTTATGT
ACACTTACAATCCATTTACTTGGAAGAGTTGTTCTTTCTAGGACTGTTCTAGGCACTTCCATGGATAATAAATGCAAGCGACCTCCAGCCCCTG
CAGGCAGGAGTTGCTGAATGCTTCCTGCGTTTCCCCCTTATCTTAACTCTTCATCAGCTTTGATGCATCTTGCAGGCGTGCTCTGAACATGACA
GCTAGCACACTCTGGGTATTTAATAAGCAGCCGATTCATGAACTAAAGCTACCCTCAAAGGTGAAAGCAGTTTATTTAAGCTACAAATAAACAC
AGGTGGGTCTCAGATTTGGCTTGTTTCTTCTCTGAAGCAATGGATGGGGTATTGTTATCACCTAGTCAACTGTTGCCCGAGTCCACCTTGAGCA
AATAGAATGGACGGATCCTGTAAAAGAGAAACTGGGGCTAAGAGAACAGGGCAAAGGCCACCTCTATAGCACAGATAAGTTTCGCCACAGTCCT
GAATGGCTTGCTAAGTCCCAATTAGCTAGCTTGAAGTAATCTCACTTTTATTGGTAGGAGGTGCTTCCTGCATGAAAGAAAAATCCACTTGTGC
ATGAGTACATGCACAACCCACACACACACTCACACACGTGCACATACCACACACATGTACTCGCACACAACATGCACACCCGCGCACACACACA
TAGATGCACACCACACATACACACACACACACACACACACACACACAGGAGCACTCACATGCACACACTCACATGCATACACCACAAACATGCA
CATACATGCATGCACACGCACACAGACTCATGACTGTATGCATATGGTCCTGAGCATCCTGAAATGCTGAGATCCTGTGCTGGGTTTAGTAAAA
GAATTGATGACTCTTCCCTTATCTTCTGGGATCGACTATAGCCTTTGACATTCTCTTTGCCTTTCCCCAGGTGAGTCACTTGAAGCCCTGCACC
CGCAGCCTGGGCTCAAGCCTCCACTCTGGCCCTAGGAGTATTCTGAGCCTGGCTGAATGCCACTCTGATCATTGTGGTTTTGTGTGGCTTTGTG
TCCTGTCAATTTACGTGTCCAGCAGGATGGCTTTCCCTTGGATGTATGTCACTCTACACCACTCAGACACCACTCAGATGAACACGCCACGTGC
CCCGGCTCTGTGTTGTGTGTCCAGTGGTCTTGGTTCCCACTCAGCCCCACCTCCGCCCACAGGAGAGCTCTCCTGAGCATCCCAGCAGCTAGCC
TGACTGCTTTTCAAGTGCACACCCACGAGACTCTGAGGCCTCCGTCCGCTTTGTTCCCTCAGTCTGGTGTCAGAGGTCCATCCTGTGCTGTGGC
TGGATGCAGAATAGCCCGTTCTGATTACTGCAGTGCCCTCCACAGTGGGGTTGCTAATGCTTGTGTGTGTCCTAGTCACCATTCTATTGCTGTG
AAGAGACACTGTGGCCACCACAACTCTTATGAGAGTTTAACTGGGGCTGGCTTGCAGTTCAGAGGTTTAGCCCATTTTCATGATGGCGGGAAGC
ATGGCGGTGTGCAGGCAGATATTGGACCAGTAGACCTACATCCTGACAGTGGGCACAGGGAGAGAGAGAGACAGAGAGACAGAGACAGAGAGAG
ACAAGACAGAGAGATGGAGAGAGACAGAGAGGAGACACACACACACACACACACACACACACAGATGTGGAGTCTGGGGTAGGCTTTTGAAACC
TCAAATCCTACCTCCTCTAACAAGGCCATACCTCCCAATCCTTCCGATCCTTTCAAATACTCACCCCCTTGCTACTAAACACTCCAATACATGT
TCCTATAAGGGCCATCCTTATTCAAACCACCACAGTGGTTGTGCTATTTGAGAAATTTGTGATTTTTTTCCCCTAAAGACTTTTGCTTGTTTTC
TTTTTTTTCTTTTTTCTTGAGACAAGGTCTTATGCAGCCCAGCTTGTCCTCAAACTCACTAATATAGCCAAGCATGGCCTTGGACTTCTGATCT
CCCTGCCCGTCTCCTGTGTGCTAGGATTACAGGCATGTACCTGCATGCCTGGCTTAGGCAGTGCTGGGGACCCAACCAGGGCTTTGTGTCTAGC
CGAGAGTTTTACAGCTACAGCCCTGACCCTCCAGGGATTTGGGGAGCATGAAGGAACTGGACTCTGCCTTTGTGGACGTCCTGCCTTCACTTCT
CTGGGGTGTGCTTAGCAGCTGGCCTGCCTGACTTAGAGGAGGTTCCAGCTGTTTGCCAAAGTGCTTTAAAGGAATGGCAGTCTCATCCTTCACG
TGGTTCCAGCATCTGCACTGTGTTTTATAATTCTAGTCATCTTGGAGAGTGACAACATCACCTTATAGTCAGCTCCCCCTGGGAGATTTGGACC
ATTTTCTTTTCTCCCTTTCCTTTCCTTTCCTTTCCTTTCCTTTCCTTTCCTTTCCTTTCCTTTCCTTTCCTTCCCTTCCCTTCCCTTCCCTTCC
CTTCCCTTCCCTTCCCTTCCCTTCCCTTCCCTTCCCTTCCCTTCCCTTCTTTTGTTTTTTTGTTTTTTTGTTTTTTTTGTTTTTTTGTTTTTTG
AGACAGAGTTTCTCTGTGCAGCCCTGGCTGTCCTGGAACTCACTCTGTAGACCAGGCTGGCCTTGAACTCAGAAATTCGCCTGCCTCTGCCTCC
CAAATGCTGGGATTAAAGGCGTGCACCACCAAGCCCGGCAGATTTGGACCATTTTCATCAGGACTCTAGGCTGTTCTGAGTGCCATGGCTGTTT
AAGCTTTTTGATTCCCCCTCCATTTTTCCTTCCCTCCCTCCTCTTTCCCCTCCTCCCTCTTTCCTTTCTCCCTTTCTCCTTTCCCTCCTTCCCT
CCCTCCTTTCCCTTGCCCTCCCCCTCCTCCCTCTCCTCCCTCCCTCCTTCTCCTTACTCTGTCAGTCTTTCCCTCTCTCCTCCTCTTTCAGCTT
GCTTGCTGGAGACAAGTCTGTGCACTCAGGGCTTGGTAAGTTAAGCCTACTTAATGCCGATACTCCTAAGAGGGACCAACTCAGAGTTGGCATT
TGTTTTGTGTCCTTTTCCTCACAAAGATATTTTGAGATCGTTAATGACCCCCATAGTTGGTCAGCTTTGTGCAAATAAATAATTTCGGGGGTGG
GGCAGAAACCCTGAACACTTGCTTGGGCTAGAGGGATAACCCACGGATGCACAGAGGCTGCGCTAGAGACAGACAGTGGCTATGAAGTCTTCAG
ACTCTGGCCTTTGTTGCCAGGGTTACAGGATTAGTACTCTAATAGCCCAGATGCTCTTGTGAGCCACAGCTGTCTCTGCATCGTGCTCCGAGGA
GGTAGTGTTCAAACACATACGAAGTGGCTTCTGGGATTTGACCAGACAGAAGTGATTCTTCTTGGGACGGGAGGAACCAGCACGCGCTGGTTTT
TCTTCTTTCACGGTGCCGGCTTCGGAACTCAGCGCCCCTGCTTGGGTTAAACATGGCCTCTACCGCGGAGCTGTACTACAGCTCAGTTTGTCGT
TTGTCTTCTCTTATTGACTCACAGGAATTTTACAAAGTGTTCTGAGTAGAAGTCCTTTCTTTTTCTTTTTGAGACAGAGGCTCAATGTGTTCCC
CAGGCTAAAAGTTAACTCCCAGTGCGAGGATCACAGGCTTGCCCCGCCACCTTGGCCTAGCCAATCCGGGTTCTGGAATCCCAGGTGCTGCCTC
GCCCTCCTCTGGTTTACCCAATCCTAGTGCTGGGATCCCAGGTGTTGCCCCGCCCACCTCTGGTTTAGCTGCTCTTTCTAGACCACTTGGTGTC
TCTCCAGCCTTGGCTCCTCCCACTACTCAGAATACCAATTCACACTTTTTTTTTTTTTAAAGATTTATTTATTTATTATATGTAAGTACACTGT
AGCTGTCTTCAGACACTCCAGAAGAGGGCGTCAGATCTTGTTACAGATGGTTGTGAGCCACCATGTGGTTGCTGGGATTTGAACTCCGGACCTT
CGGAAGACCAGTCGGGTGCTCTTACCCACTGAGCCATCTCACCAGCCCCTGGAGAGATGGCTCAGCCGTTAAAGGCTAGGCTCACAACCAATTC
ACACTTTTATCAGTTTTATTTATTGTGTGTGTGTGTGAGTGTGCATGTATATATGTGTGTGTGTGAGTGTGCATGTGTGTATATGTGCGTGTGC
TTCTGTGCATGTGTGCTGTGCATATGCGGGTATGTGTGTGCATGTGAGTGTGCGTATGAATGTGTGTGTGCTGGGAACCCCATCAGACTGCAGG
TGGTAGACCTGCATAGATGAGAAAGGCTTTGAAAAAAATGCAGGGTTTTTTGGGGGGGGGCACACATAGAGGGTAACATGCTCACTGGGGAGCA
ACCTCCCTACACAGGAAAGGGGTTGGCAAAGCTTGCTTCAGTAGGACACAGGGGACTTTGATCACGAACAAAGAGGAGTAAGTTGGACTACACA
CTCACATACATACACACATGCACATGAGTATACAGACATACACATATACACACATACACACATGCACGCACACATATACATACATATACACACA
CACATGCACACAGATTTAGAATAAATTTAAAAAAATTAAAGCATGAAATGTAATTTTTAAAAGATATCAGTGTGGCTGGAATCTGGGTTTTTTA
AAATTAAGGTCCATGAAGGCCGGCATAGGAAAGAGGTCAGAAGCTGGATCTTGAGGAGAAGGAGGTGGTAGGAGAACAATGGCTCCCCAAAGAT
GTCCAAGTCAAAATCCACAGAACCAAGAATGATGTCAGGGACTGATACTCCTCATCTGACGTTGACTAGAGCAACGACTCTGGGTCATCCAGCT
GAACCCAGTACAGCCACGAAACCCTTAGTGCAGTGGCCCTCAACCTTCCCAATGCTGTGGCCTTTTAACCCAGTTCCTCCTGCTGTGCTGACCC
AGACCATGACATTATTTTGCTGCTACCTCATAACTGTAATTTTGCTACTGATATGGATCATAATGTAAATATCTGATATGCAGACGGTCTTAGG
CAACTCTGTGAAAGGGGTCCCGACCTACAGGTTGAGAACCGCTGCCTTAGTGGACCAGGGAGGTTGTAGAGAAGAGCCAGGGACATGAATGCAC
ATTTGCTGGCTTTGGAGATGAACGAGCTCACAAGCCAAGGATTTCCCTGGACATGGCCTCTCTGAGCACCGATTCTCCTCAGAAGGACCTGCTG
ACACAAGTGGGTGAGGAGTGCCTGTCTTTGTTCTAGTGGCCATACACAGTGAGTGGAGAGGAGGCAGTTTTTAGGTGACAGGAAGGTGTGTGTG
TGTGTGTGTGIGTGAGAGAGAGAGAGAGAGAGAGAGAGAGAGAGACAGAGACAGAAAGGGGACAGGGACAGAGGGACACGGACAGAGAGACTGA
GACAGGGACAGAGATTTGGTGACAGTACCCATAGCAAGGAACCTCAGAAAAAGCCTGGGTCACAGCAACAGCAGCCTGGTCTCTCTGGGTCGGG
GTGTGCTCTCTGGGTGTCTTAGTCAGGGTCTCTATTCCTGCACAAAACATCATGGCCAAGAAGCAAGTTGGGGAGGAAAGGGTTTATTCAGCTT
ACACGTCCACATTGTTCCTTATCACCAAAGGAAGTCAGGATTCGAACTCAAGCAGGTCAGGAAGCAGGAGCTGATGCCCAGCCCACGGGGGGAT
GCTACTTACTGGCTTGCTTCCCCTGGCTTCCTCAGCCTACTTTCATATAGAACCCAGCAGTACCAGCCCAGGGATGGCACCACCCACCATGGTC
TGGGCCCTCCCACCCTTGATCACTAATTGAGAAAATGCCTTACACCTGGATCTCATGGAGGCATTTCCTCAAGGGAGGCTCTTTTCTCTGTGAT
AACTCCAGGTTGTGTCAGGTTGTCCCAGAGAACCAGTCAGTACACTGGGTCTGGAGGACATGTTTAATTGCCTCCTCTGGAGAGGCTGGGAGGT
GGTGATGGACTCTCTACAACACTTCGGGGCCTCTTCTCCTTAGTTAACCCCTGCACATGCCTCTTCTCTTATGCAGTCATCTGACCTAGCTCCA
CCCCCGACATATGACTTATGCCAGGAGATCCTACACTGAATCTCTCCTGAGTGGCTATCGCTGGCGTGTGGGAAATGTACACGTGGCTCACATG
AGACACCACCTTCATAGCTTCACCCTCAAAGACCCCATCAATATTCAGCTGTGAGTCAGTGCCCATTGTAGGTGGCTTGGGTTCTCTTAGGTCC
TGAGTGGAGCTATTTCAGACTTCTAGCAAAACAGTTAGAAAAAAATAGACCCCACTCACACCCCGATCTATATGGCTTCAGGACAGTTGTCAGG
CAGGCTGGGCTGGTGTCTTCTAGTGTGTACAGAGACACCTGCTGCTAGGACCAGAAGCTAGGGCTGGTGTCCCTAATCTCTCTCTACCTTTAGC
TAAGAGGCAGGCACACTGCGTGGACATTTGGGATCTGTTCTGTGCCTTTGATATTTGTAAGTAGGAAAATCTGCATACGCTTATAAGGTGCCTA
TTTGGGAAATGAAAGGGGTATAAATGCACACAGGAGTCTGTTGTAGTAATCTGAGCGTGTCTGACTTCTGGACTCTGCCCTTCACAGCAAACAC
TTATTAAAACACTCCCATATTACTATTAAAGAATTTGAGCTGGACTATGGTGGCACACACCTTTAATCCCAGCCCTTGGGAGGCAGAGGCTAGC
AGATCTCTGTGAATTTGAGGCCAGCCTGGTCTAGTTCGGGGACAGCCAGGGCTACAGGGAAACCCTGTCTTGGAAAACAAACAATAAACCCAAT
AATAATAGTAATAAAACAAATGAGCTTTAAAAAGTAGTTTTCATTTGAAATTCCTTGGCTCCAGGGAACTCCACTTGGCTTTTGACTGTTGGAT
GGTATTTATCATTGAACTAATACCCGGTAATGTCTTAAGTGCCTTTCAGGTATACCACAATGCTCATCAGGATAAACTAGAGTTAGAAGGTTCT
GTTTGGAAAATCCTTATTTGAATTCGTATTTATTTGAAACCTAGCCGTATACTTCTTGTATTTGTGGACCAATCAAAAATTCTTGTCGCGGCTC
GTTTGGAAAATCCTTATTTGAATTCGTATTTATTTGAAACCTAGCCGTATACTTCTTGTATTTGTGGACCAATCAAAAATTCTTGTCGCGGCTC
CAACGTTTTCCTAAGTATTTTAAAAGCCAGACGGGTGAGAGTCCTGTAAGCGTGGCCGCAGGTGTGTAATTGGGCAGCCTGCAACCAAAGTCCC
TCTCGACCCTCACCTAAGGCTCCCCGCTCCCACATCCTACTCAGGTTGGGAACAGGCTCGACCGGGCTCCTGACATCACTGGGGGACACAAGGG
AACCGATAATGCGTAGCCTTCCTGGGGGCATCCCAGCCTCAGCGCTCGCGACTCCCCTGGTTTCCCACATTTCCTGCGCAATTTCAGGCGGTCA
TCCTCCATCCAGGAGGGCGCGCTGCAAGTGGCTGTCACCCAGCACGCAGCTCTGGGACTGGTCTTTGCTCTGAAACTCCAGCCTGAGCAGCTGA
CACTCAGGGTGCCCCTGGACACGTGCCCGGGGACAGAGGCTCTCCAAACCTGAACGACACCCGAGGATGATCCGTGCATCACCAGCCTCCTTGA
CCTTAGGCAGACGCCCCCAGCCCCCCGGCCATTTTTGGGGCAGCCCCCTCCCCCGCCGCCCCCGGAGTAAAGAGAGAAAAGGACTCCACGGGGT
CGGGGACGTGCAAAAGTGATTTCTCGGAAAGAGAGCGCTTCAGAGAAGGCGGATTTGGCAGGCTGCGCTGATTGGGCCGCGCCACCGCCCCTCC
CGCTCCTCAATTAGCTCGCGCGACCGTCGCTCTCCGCGCCGGACCCGCCTTCTCCCCCGCCCCATCTATAAAAGCAGGCGCGCGCGTACGCGCC
TCCAGGAGCCCGCGGGCGGTCCCGGAGGCGCGGGCAGCGTGGGAACCGGCGGCAGGTAGGACGCGATGGGGTGGGCGCGGGGGCCGGGGCTGTG
GCTCTCCCACCTTCGCCCAAACGGAGTGCACAGCACGTTAGCAGGTGCTTGCTCTGGCTGCTCTCCAAAAACAACAACAACAACCAACAACAAC
AACAACAACAAAACCCAACCAAATGAACAAACACCTCTCCCTTTAAAATCTGCCTGATGGCCAACTTCATAATGAAGAGAAATAGATCTGCCAA
TCACCGCGCGGCAGCCCGGCTTCCATGCATGAACTATCCAAAGCTCGCGACTGGGCTCGCCCAGGGACCGACTGGCTTTGATTCTGACTAGTGG
CAATCTGATTGCCCAGTGGCGCTGGACTTCTCTGATTGTCCAGTGGCCCAGCTGGCCCCGGAAGGAAAGTCTTCCGTGTGGGTCCCACACAGCT
CTCAAGACCAGATTTGCTGTCAGCTTGGCCCCAGGCTATAGAGQCAGACTTTTCCCCTGGTGAGAAAGATTACTTACCCACAGCCGGGAAGGGC
GTTCACGGGCTAGAAACTAATAGAAACCATTCACTAGAATCTTAAAATGGTAGATCACTGTTTTGTTTGTTTTTGTTTTGTTTCTGATACAGGG
TATCACTCTCTAGCCCAGGCTCATTCCAATGAGCTCCCGGTTATCCGCCTACCTCTTCTGAGTGTTAGGATTTCAGGCGGAGCTTGGGGGGTGC
CCAGGTGCTCAATGGCCCTGTTTTTTTCTTTTAAAGTCAAACGATAAGAGCAGTTTCGTGACAGTTCTAGGTTTGTCCCTTTAAGTTATGGCAG
GCTCCTTCATGAAGAACTGGGACCGATTCAGTGTCCCTGAGGAAGCTTGGGGCCAGAAAGAGATGGGTCTGTGATGGATTTCCGTTCAGTGGTG
GTCAGACTGTGGCCCAGGGCTGAATGAAAGGGATTACGGTGTGGTGTGAGCCCCGCCAAACATGGCTGGTGCACATGTTGACAGCTGTGTTAGG
CGAGCCTCTGTCCCCTGCCTTGTGAGAAGCCTCACCTAAGACACGTTGCTCTAGATGTAAGTTAGCTGTAGGCAGGATTGGGCCCACAGGGAAT
CCTGCCTCAAAATCAAGAAGAGCAAGCTAGAATCATGTGAGCGTGTTTATAAACCATTAATTGTCCATAGTTGTAACAGAGTCAGGCTATCCCT
GTCTAGGACCTCACATTGGCCTCCAAGGCAATAGAGGAGATTGTCCAGGTGCTGAGAGCCGGACCCTGTCAGGTGTTCTTTCATCTGCAGAATG
AATGAAAGAAAGCATCCCTTCTGTGTGTACTGTCACTGGCACGCTGCTGGCAGTTTGTGCTGTGCCTGCCCACGGGATGGTGGTCCCTCCCAGT
CTCTCCCCCTTCCGAGTGTAGAAGAGGCTGGGTTAGAGAATTTTAACCTCCTAGCTTCCATGGTCTGCCATTCACCTTTCCTTTTAAACACCCC
TTTTCTCTGTCACCCACCAAGACATCTGTCTTTCACAGTTCTGGGGTGTTGGGTACCCCAGGATCCCTGACAAGAAAGTGCCATTCCTGTTCTG
GAAAAAACTCCAGCCATGCCAGGGAAGTCCCTTCAGATTTCAGGAGGCTCAGGGACCATGGGGGTATGGGTCACAAATCTATCTCCAGGAGCTG
AGTTGAGGCTTTCAATGGACACATCCTCAGATACTCTGGACCCCTGACCCCAGCTCCCACCCCACCCTCATCCTTAGAGGCCACACAAGACACT
ACCAAACCAGGAGAGCCTCCTATCTCCTCTCCAGAACAAGCTGTTGGAGCCAGGACTCTACTTGGTTTTAGGGGTACCCAATGCCATTTCAGTT
CTCTCCCTAGGTATTCCTTTCTGACTTGTCACTTATGAACTCAGTGGTCTTAGAATACTGGGTTTCTTCAGATAACCTTTCTTTCCTGCCTTTC
TTGTGAGGGTCCTTAGACTTAGGAGCCAATGGCTTCCCAACGTGAAGAGCCCCTGCTGAGGTCAGGGTTTCCCAGCCGTCTTGATGATTTGCTT
GAGAGGTGCCTCAGGTGGCACCGCACTTGCCTAGCAGGCACAAGCTCTGGGTCCAGTCCTCAGCACGGGGGAAACGGAGGCAGAAAGAAAGATC
AGGCGTCCAAGGCCATCTTCATCAAACTAGGGAGTTGGAGATCAGCCTGCGATACAAAACCCTGCCTCAAAAATAAAACTCAAAACTGACTGGA
GTCTGACTCCGCTTTAGCCCTCGCTCCCTTCCAAGCCCACTGGTGGCAGTTAGGGCTGCTGTGTGGCTAAAACCAGCCCTGGAGAGACCTCATC
TCCCTCCTCTCAGGCTGCTGCCGTCTTTCCTGGCCCCACTACCACCTGCAAGCGGCCTGGAGTTTCTGACCCTCAGGCCTCTTTCCAGCAATCC
TGACTTTGTGGGACATGCTGGCTCTGGATGTGACTATGTTCTTGGGGATATCTTGACTTCTCACTGATCATAACTTCTTTTTTTTTTTTTTTTC
CCTACCCCAAGAGGTCATTTTAGTTGGAAGACTAGAAGTGCTGTGTTCTGGTTTTCAGGGTGGGGCGTCTGTCCAACTGCTTGGGGATTTCCAG
GCTGTTCTAGTAAATGCTGGCTGCAAGTCCCTGTGCACAAATGCCCTCTCTCCCCACGTGTAGGAAAAGCAGACCCCGAGAGTGTCCAAGTGTG
CCATTAGGTGTCTGGGGATGGCGGGCCTGGATATGGAGTCCCTGAGCTGGCTTTGTACTGTCCCTTTGTTCTCACTGAGGGCTGATTGTAAGAG
GGACTCCCTGTGTTGTTGGAGGAGTCCTCTAGGATACAGGCTCTGATTGCAGGTACTGTACACACAGCTCCCAGACCGAATCCTTAACACTCTA
AGGTTTATTGGCTTCAAGGACACCCAGCCAGCACAGGACTCCAGCTCTAACTCCATCCCAGTGGGAACAGGACCCTATACTGTCTGATATTACT
ATTACTATCCTAGCTGGCAAAGGGCAACCTGCAAGCAGCCTTGAGGGTGTCCTCTTTCTCGGGTGGTGCTGGGATCAAACTCTGTGAGGAACAC
TGGCCCCTTGGGTATCAGTAGTCACGCTGGTTCTTAGAGAGGCGAGCAGCTCAGAGAAAGCACAGCATTGTAGGAGAGGGACTTCTTCCTAGCA
GGTGCCCTCAGTGTCTGGGACAGGGGGTCAGGGAAGAGCTGGACCCAGAGCCAGAGCTGTGGGTGGGGCACATCTGTGAACAGCCTTTCGGTTT
TCCTTTGGGTTTAGGGCCACCGGGCACTGCTGATTTCTCATGGCACTGGTCCAGATGTCTTCCTGAGCCGGCACCTTTGCGTGGACCTCTGATC
TTCCGGCAGTCAGTTCCGGGACTGCTCTGACTCACAGAGATGTATTTGTTATCCTGAAAGATTTGCTTTCTTTCCAAGGATGTGTGACCGGAAC
GGCGGGCGGCGGCTGCGGCAGTGGCTGATCGAACAGATCGACAGCAGCATGTACCCGGGGCTGATCTGGGAAAATGATGAGAAGACCATGTTCC
GTATCCCCTGGAAGCATGCCGGCAAGCAGGATTACAATCAGGAGGTGGATGCTTCCATCTTCAAGGTAAAGCCTCCTGCGTACCCACTCAGTGG
TTTTTGGTTTCCCTCTGGCTCTGGAGGTATTTAGGGATATCCTATGACTAAACAGCCCACTATTTCAAGAGAAACAAGGAGGAGTGTCTGATTC
TGGCACGTTCCCTCATGAGGTTGAGAGTGACTTTCCTGCCCATTCCGCTCTTCTTGCTGCGGTGTACTGACATGCTCTGCTTGGTGTACTAAGG
AAGGCTTTGCTCTTTCAAAGTGCGACTCCTTGGCATGCAGGGATCAGCTATAAAGGGCGACAGGCTTCCACAACTGCTTCTGGGTCAGGTTTGA
GGGACACCCTGAGCTTGTGTTTTAATGTTTCCCCTCTGAGAGGATTGAGTGCGATCACCGAGGGCAAGTCACACAGCTGGCCTGAGAGGAGTCG
GCACCCTCTCATCTCACCCTGGGTGGCAGGTGATGCTCCTGTTTGCACTCTGTACTGCCACGGAGAGAAGCCACTTCTCCGGGATCTCATAGCT
CCTGTGTGATGGAGCCTGTGTTTGGGACTGGGTGGTCCAAGGTCTTAACCCTTTATAGTATAGAGAACTAGGGTAATTTCTCATTAAGGACCAG
CCCAGCCCAGTTCTGCAGGCAGGCAGGGTCAGACATCCCCTGAGGGTTGTTACACTCATGTATGAGTGTCCCTAAGGTTTTAGGCACCTACATT
CGTCTCTGGGAGTTTCAGAGTCTTAGGTCATGGCCAGCCCACACTTCTACTTTGTGAACAGCTGCAGCCTTGGTGGTTTGACCCAGAGTCCAGG
TTGTAAACCACTGCCTTTAGCTCAGTATGTCTGGGGCAGGGTGGAAGGTTATGAGGCCCACTGAACTCAGAGGACCAGAAGCTGACTCAAGACC
GTTTTCAAGCAGGAAGCAGGGCCTGCAGAGACTCTGACTACCAAAACTGTCTTGGATGGGTCCCTCCTGGGGCACAGTCTTCCCTTTCCAGTGA
GAACTCTGAACCAAGCATATGGTGACTAGCATTGACACTAAACTCGGAGGCACAGTGTGCTTGGCTGAAAGGCCATCACGAATCTTCCAGAAAA
AAGGGCAGAGTCCTGGACCAGGGGCCTCAGTTGTTGCAGGAGACAGCAGGCCTCGCTTCATCCAGTTCCCTGCTTCAGCAAAGCAGGAGTCCTG
AAATAAAGCCCCAGATTTATGCATCTTATGCAACGTTCTTCACGGCCAGCAAGCCGTTTCAACAGCCCTGAGGAGAATGTGCAAGATTCCCGAT
GTTTCCGTTGGTTACGCAGTCGCTCTGTGTGGCAATCTGAAATGAGACATCAATCTCAGCAGGAATGGCGGGAGATCAACGGGCGGGCGGGCGG
GCGGGCAAGTGGCAGGAGATTCAGGGCAGAGAGGGTCTTCTCTGAGCTGTCCTGAGTGGGGCGGGTACCTCCGCATCTTCAGTCAGACTCAACT
TGACTGTTGAAGGAACCGTAGGATGAGGCCTTGGGCAGATAGAGCCATCTTAGGGGCCGTCTGCTTAAGTAAGAAACCCACTTTTCTCAGCCAG
TTAAAACCTTTAAGAAATGTAGGTGTTAAGGAAAAGTTATATGCTTGCTTCTCTACCAGGGAAAGGGATTACACCAGAGAGCCCTTGTGCTGTC
TCCGGGCAGCGTCTTGCGGCTTGCTTCCTGTGTAGACCGCAGGGGGGAGAATGAAAAAGATCCTGAGCTTACCTCAGCCAGGCAAGGGCTGAGA
TTCCTTGGGCCTCGCTCGAGATAGGCCTGACCGTTTACCACTTACTCTGTGTGCAGAGGGTCTTGAAGGACGGAATAGGGGTTAATTTTCACTT
TACTTTATTTTGAGTTTGGAACTTAGGTAGTTCAGGTTGATCTCGAACTTGCTCTGTAGCTGGGTATAACCTTGACCTTCTGATCTTCCTGCCT
CTATCTCCTGCGTGTTCGGATTACTGGTGTGGGCCACCATACGTGGTGTATATGGTGCTGAGGATTGAACCCGAGGCTCTTGCATGCCGGACAA
GTACTCCTTTATCAACTGAGCTACACTCCTAGTATTAATTTTTCAAACACAGTGCAACACAATGTCAGCAGAGAAGAGCTTCAAGCAGTGGGCG
CCCCTGCGGGTCCCAGGACAACTCACTCTGTGCCTTCAGCCCTGAGAGACCATGCAGCTAAGATAGCCCTGCAGAGATGGTTTCTGGCCAGATG
TGGCAAGGCTCAGGGTAGGCCTGTGCTAGTACCCAGTGCTGTCTCACGATGCTCCCTGACCATGGAGTCTGATTTGATTGGTACCTGGTATCTC
CAGGCTTTCCTCCCCGAGGAAGCATGAGACCAGAATGGAGGGATAAACTGGGCAGTACTGCTGAGATCCCACCGTTCTGGCATGCGCCAGCTGA
CCGAGATACTGCTGGGGTATCTGTGCTTTCTCGGGTGCCATTGGAGAGTGGGTTCACCCAATGACTTAAGGGGTGTGGTTTTTCACTTGTGTGC
CAGATATTCTGGGCTGCTACATCCAGGGAGAGGTACTGCTGGGGAATGCCTTACCGTGTAGGGTGGTCTTCACTGTGTCTGTGGCAAAGAGGCA
GAGGTTTCCATGTAGCCCAGCCCCAAAGTCACTGTTCCAGTCTGCTTTGGAGTTCACAACAGCCCACAAGTCAATGTGTAGCCAGCAGTGCCAT
TTACAATAAAGGCCAGAATTGGAACTGTCTACTTAAAAAAATTTTTTCTAAAAGTGGGTTTGCAGCCATGTCAGAGAGGAAGTGACCACTCAGG
CTCGGGGGTAGGAATGTTCCTCCAGCTTCTTGCTGAGCATAGATACAGAACTTGGCATCAACAGTGCAGAGAGCATTGGCCTGGAACATAAGTG
TCCCCGGTAGGTGTACATGTGTGTGTGTGTGTGCATTTGGAGGCCAGAGGTCTATCATTGTCCACAAAAAACGATTTCACTGAACCTGGAGCTC
AGGGATTAGGCTAGGTTGACTGCCAACGAACCCTGGGGATCCTGTTATCGTGACCCCAGGGCTGGGGTCACAGGCACACATACCCATCTTTTTC
TGTGGGCACCGGGGATTTGAACCTGGGCCTTTATGTTTACATGGTAAGCACTGTATTAAGTTGGCCCCTGCTTTGAATTTCTGGCCTTTGCTTA
AAGCACAAGCTTTGGATGGGTCAGAAAAAAGAAATTAAATAAAAAGAAAAAAAAAAAAGAGGCACTTAACAGGGACAACAGTTGGCTGCATCTT
AAAGGGCTAGAATCATGCAAAAAAAAAAAAAAAAAGCCCAAACAGATGTTCTATTAAGAGGAGAGACCGGTGGATACACATGTGTTCCTTTTCA
TGCTGGAGGTGTTATGTGACAAAGCAGAAAGTCTTTGACGGTGCTGGTGAAGCCATCCACATTTTCCTCTTTGGACTTCATGACCTAAGACACC
CCTCTTGCCTCCTCCCCAGGTCCTTCAAGCCAGAGGACTTTGCCAAAGCTGCATCCTAGGCTGTGGGGAGCCTGGGTTCAAATTCCAGCTTTGC
TGAGTGTTTTGGGGCACCCTTACCCAGCTCTTGTCTGTTGAGATACAGTGGTCTTCCTGAAGTCCAATTGGGTGCTGACCTTGGACAAGGGTTG
ATGGCCAGGAAAGGCTCTGGGGGAAGGGGGCGGGCGGTATGCAGCCGGCCTTTGAGTTGAACTCTGTGGTTTGTGTCCCTCTGTGTTTACTGAG
TTTCTCTCTCGTTCTTCAGGCCTGGGCAGTTTTTAAAGGGAAGTTTAAAGAGGGAGACAAAGCTGAACCAGCCACGTGGAAGACGAGGTTACGC
TGTGCTCTGAACAAGAGCCCAGATTTTGAAGAAGTGACTGACCGGTCCCAGCTGGACATTTCTGAGCCATATAAAGTTTACCGAATTGTCCCCG
AGGAAGAACAAAAATGTAACTATCTGTTGGGACCAGGAAGCCCTCTAGGAACCTCCTTCACCCCCCCCCCCCCCACGCTTAGCTCCTGAAGCCC
TCAGTCTCCACAGGGCTAGAGCAGACATGCCACCCACAGGGTTTGGGTGCCTGGTTTTAGCTCACCAGAGTTGTTCTCTCTTGTGTAAGCAGAC
ATAGAGAGCTGCTCTGCTGACCTCATACGCCATGTGCTGTCAAGTGGCAGCTTCCTGCAGAGGGAGTGGCCCAGTGGAGGCAGGGAAGGGTGGT
GGACTCTGGGTCATGACAGCCAGAACCTGGAGGGAGCGAGTCCCCGGAGCTCAGCATAAACTCCTCCAAATGGGTTPGAGGCTCTGCTGAAGTC
TGTGATGCAGGATTCCCTGCTGCTTCCAGGCAGTCTACAGAGGAGGCTGGGGAGGAGTGGGCAAGGCAGGGACGGGCGGTAAGGTCCAGGGAAA
AGTTCTCCAGTTAGACGGGCCGGCAGTGGGGACATCACTGATGGCTTCGGTGTGTCACTGCTGGAGAGGATAACATCAATGCCGAGGTCTTTTC
CAGCGGTAATGTCCTAAGAGCATCTGTAAAGGAATGAACGCACACTGGCCTTCCCCAGCAGAGCCTCGTGGCTCCCAGGGCCTGGTCCGTTCTG
TACTGCTTCTGGAAGGCCGGCTCCTTTGTCTTCGCAGTGATTTGAAGCAATAGGGAAGTTTCTTTCAGAGCCCTGCTCCCCCAGGACCAGCCAG
GCTTGATCTGGTTAATGACCCTGACTTGTTTATAGTGTGGGTGGAGCCCCCTCACCTGCAGGTTTGGGGGTGAAGGAGAGGCAGAAGGACATAG
CATTTGCATGGCATTTGTCTTGGTATACTGTACTGTGAAGTTGAGTGGTTTGCCTGCCTCTCTGGCTGGCTGGGTGGGTGGGTGGGGCTCTCAA
CCTCTGCTTGCCCAGCCATGGCTTCTAGGTGAGTCTCTCCACCTGGATGAAGCAATGGGGAGAGATGATACAGGTTCCTGGGGGTGGGGAGCCT
GGCGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNACAAAGTGGGTCAGCACCCATCTGGGACCAGGTATACTGGCCGATGAAAGCATTGGTCCCCTGGGGAGGCAGGCAG
CAGGCTGTGAAATGTCATGTGTGTGACTTCCCCATGTGGCACGTATACACACACACACACACACACACACACACACACACACACACACACACAC
ACGCACACACGCACACACGCACATGCACACGTATAAAAGGGCAAAAAGATTTTCTGTTACCTGGCACTCCCTACCTTCCAGGAAATCTCTGGGT
GTTTGATAAAAACCCAGCCTAGCAAACTTACCTTCAGTATATGGGCCATAAATGCCTGGTATAAAATGCACCCGGTGGGGTTCAGATAGAAGGA
AGAAGCCAAAGTCTGGCGCTTGTCCTTCACAAGCCAGTGTTTACAACCCCTCCACCACCCACCACTTCTCCCTTTTTGACTTGGCAGATCAGAT
GACCCGGCCATGAGAAGCAAGGGCTGAGCCTTTCCCAAACCTTCCAGCAGCTGCTGGTCAAAACCAGCGAGGACGCTCACTACTCCCGTTTCCT
TAATTCAGCATTTTAAGAAAAGGTTGCAGTGTGCTCGGCTGCCCTGGGTCATGGTGTCTGTCCCCTTTCCCATAGGCAAGCTGGGCGTGGCACC
TGCAGGCTGCATGAGCGAAGTTCCTGAGATGGAGTGTGGCCGCTCAGAGATTGAGGAGCTGATCAAGGAAGTGAGTGGAGGGGGTCTCAGGCGT
CTTCCAGGGAGGATGGGACATTGCTTCTAACCCTCAGAGGCACTGAGAGCCTCAGGCTCTGCAGGGAAGTGAGGGACACCCCAGCTACCTCCCC
AGGCCAGCCTCCCTTTACTGGTCAGGCCCTGCCCATCCAAGCATAGTTAAACCCATTGAATAGCAGCTTTGAAGGTCTCCATGGAGTCTTAGCC
TTGTTTCTTAGCCAAGCTTCATATGGAGGTCTCCATCGCTTCTGGTCATCCCGTCGGCCCTCATGGTAACTAGATGGCTTGCTTTTCAAGACAC
AGGTTTTATTCCTGAGTGTGTTATAAGATTGGATGACTGAGGCCCGTAGGATCCACTAAAGGTGGTGGGGACCAGCCTGTGGCTCAGCGCTCAC
CTATCGTGTACGACAGTTCCCAGCAGCATACACAAAACACACATCTTAGAATAATCCCCCATCACATGCATGGTTGGCAGCCTGGAACTGAAGG
CTCAGTGGCTCCGTTTAAGTTAAACAGATGGCTTTTTTTGTGTGTCTGTTTTTAGCCTTTATATTGTGTCTGTACAAGGTGGGGTTTCCATAGC
TGAAACAACCCTTTAAGCTTATATAAGCAAGATGAGGTCTCTTTAGACGGCAGCTGGCCCAGACCCTGTCACCTCAGCTACTCAGGAGGCGGAG
GCAGGAGGGGTTAAGACCTGCTTGAGCTGCAGAATGAATTCAAGGTAGAAAACTTCACGAGACCCTGCCTTGAAATCAAAGAGGGAAAAAGGCT
GGCAATGACATAGCTCCGTGGTAGAGCGCTCGCCTCGCGTGCACAAAGTCCCGGTTTCCATCCCCAGCTCAGTGACATCATAAAAATAAAACCA
AGACTGTTGTTAGCAACGACACATCAGCTGTGTGGTCTGCAGCATTCTGTGGAGCACTGCAGCCGGCCAGCATCACCTTCCCAAGCTGCCCAGC
TCTGCGGCACATGGCCGTTGTGATGTTCTGGGTGGTCTTGGTTCTGTGTCTCCCCCACCCCCACCTTGTCTTGTTTTCGGTATAAGCCTTTACT
GCCAACTTGCCTGAGCCAGTAGATGGTGATGATGTCACACCAGGGTCACAAGCAAAGGAGGTTTCAGGACGTCTCCTAATTGGACCCATCCACC
ACTGAAGGCCCGAGAGCCCTGTGGCTCTCTGAGTGAGATTCTCAGCACAGTAGAATTGGCTTGGTGTCACCCCAGAGGGTTGCCCCAGAACTGG
GCTATGTCTCCCAGCATCTGAAGGCCAGTGAACTCTTTCATAGCCAAGGCTAGTCAGGATGCCAGCCCAGTGGGTTTGTAGCTTAGGGTCTGAT
GAATTGCAAAGGGAAGCTCTGCTTTTCTCTCATGAGATAAAAGTTGTGGATTTAGTTGTGTGTCTGATATTGGGTTTTACAAACATGGGGTCAG
GTGAGTGCTGTGCTGGTGACCCGGGACTCAACTGCTCTTTTCTGTCACCTGGGTCTACAGCCTTCTGTGGATGAGTACATGGGTATGACCAAGA
GGAGCCCATCCCCACCAGAGGCCTGCAGGAGCCAGATCCTCCCTGACTGGTGGGTCCAGCAGCCCAGTGCAGGTGAGTGTGGCTGTCCGGCACT
AGCAACCACTAGGCTCTTTATGTTAGGGTAGGAGGTGCTTGCCACTGCTAGGATCCATGTCCTGTGGAAGGGTCATGAGTCACAGCCGGGGACC
GAGACAGCCAAGCTGCAGCCTCTCAGGCAGGCACAAAGGTCTTGGTAGTCATGCATGGTCAGTCCCTCCTGTAGTCCCCCGGAAGCTGAGAGGA
GCCTGCCAACATCCCCAAGCCTCAAGGGAGGTCCCAGCTCTCAGCCACCTGTCTCTTCCTGGCTCACTGTCAGGGCAGCCCCAGAGCCAGAGAG
AGCCAGGGCGTTAGGAGCGGGAGGGGAGCTCAGTCAGGAATAGGCGTTAGTTTGGAACCACTTTGGGCAGTTTTCATGTCTTGGTGGTGCCTCT
TGTTTGCTCATCCATAAAATGGGCCACCAGGACCTGCCTGGTAGGATCCACAAGTTAAATAAAGGATTCACCCCTTTGATAGTATCTGGGAGGT
ACACCCATGCTGTAGCTACTTAGAGGGTCATTCACCTGGGCCCCCTACCTCAGTGTGTGAGCAACTTTTATGGCTCACAGACATCTTGGTTCCT
CTTTTGCCTCTCTCCTGCTAAGACAGGAACTGCTGGGAGGTCTTAACTGGCCTTGCAGGACTGTGAGGTTTCCAGGCTGTGGGTGTTAGTGTCC
TGCAAGGGGCCTTGCGCTGCAGGGGTGTGTCTTTGGCAAAACTGCCTGGGACTCCAGCTTCCTTGTGAGACAAGTCCCCAACCATTGTGTATGG
TCCTTGGGGTAGCAAGGAAGCTTAGCACAAGGACAGGAACCTGTAGTGCAGAGGAAGTCCCCTCCATCATGGTAGGTTTCTGTGTCTCACGTAG
AGTGAGGTCTGCCCACTAATGTTGACACCAGCAGTGTCAGCCTCTCATCTATAGCTCAGGGATACTTGGGTCCTCCCTGCACCAGCCCAGCAAG
GCATATCCTTTCTTTTCTCTGCTTACAGGCCTGCCACTGGTGACCGGATATGCCGCCTATGACACACACCATTCAGGTACCAGGGTACTCTGGG
AGAGCATGGCACAGGCTCTGCTTGAAAATAGCCCCATGATGTAGATGGGGTCATGAGGTCCCACAGACAAGTGTGGCCTAGGGGTAGGCTCCAG
AGACTGATTCTTCTTCCTCGTTCTGTGTGGTTTGGGAGCAGTTATAGTCATCCAAGAGTGTCCTCAGGGACAGCTCTGCAGCCGGGAATGTAGG
TTGCTGGACTGCCTTGGCCTCGCGTGGTGGGATCTGACCTCTTGGGGCGTCAGGTTCATCATGTGTGAAATGCTGTACTATTCCTAACACGGTC
TAGTAGGGACACAGCTGGCCAGTGTCTCCCATGTAGGGTCTGTCACCTCCTCTCACTTCCAACTCTGTAGCAAGAGTCCTAGAGATGGTTGTAC
TGATATACTGATGGTGCTGAGGCTGGTGCAGATGTTGGTGGTGCTGATGGTGCTGATGTAGATGATACTGATAGTGCAGATGTTGATGGTGCTG
AATGGCTATGATGCTGCAGATGCTGCTGATGCTGGTATGGATGCTGATGGTGATGATGTGGTTGCATCAGTGCTGTCTATAGAAGTTGGACCTG
GTTCCTCCTCCCAGCCCCCATGCTCCCAGACTCAAGATAATATTTACAGATACCTGTATGAAGCTAGGCTCTTCTCTGACTAGATCATAACCTA
AAATAACCCATTTATTTTAATCTACATTCTGCCACGTGGCTGGTTACCTGTGCTCAGGGACCATGTGTCCCTCTCATCACAACTTCCCGGGAGA
ATCTCCCGCACTTCGCCCTATCCCAGAATTTTCTCTCCCTCCGGGATGTCTCTCTTCCTATTTCCTGCCTAAGCCATAGGCCGTCAGATTGACT
GTTTCCAGGAACAATGTCCATACAGATGTATGAAGTTCCCTCTACAGCTGCCTTAATGAGTGAAATGATTCTTTCTCCTGGAGCACTGTAGATG
AAGAGCGAGCCTTCATCTGAATTCTCCCCTCCCTCCCGCTCCCTCCCCACCTGCGGTCTTCTGGCTGTCTCCTCTGCGGTCTGTGTTATTTTGT
CTCTCTGTTGTGTGTGTCTGATGTCTGTCTGTCCCTGACAAAGTGCTTTGCTCTGTATTTATGGAACAGCACAATAGGTACAGTATGGGAAAGG
ACTAAGGGATTATCTTTTTATAGAGATCTGGAGTTTTTTTTCAAGTTAAGACGTTACTTTGCTGACCCCCGCTGACTGAGAATAGCGTGCGGCC
CTGCAGACATGCTATTGATTTATATCCTCGTGTGCCTTCCAACTCTTAGGGGATATTTCTTTTGATTATGGTTGCTTTTAACCTATTGGCTGTT
TTCAGCGAACGATTACCATAGTGTGTACAATGTATGCATGCACACACACACACACACACACACACACACACACTACTCTGGTCATAGGCACGGA
AGGGTATATAATTTGAGATTTAAGCTATGCATTTAGCTTGTGTTTTAAAAGCTGATTGAGAATCTGGGAAACTGAGGCGACATAAGGTTGGTTG
CATGGGTCTTTCAAAGGCAGGTTAAACGGGCCGAGAACACAGTATGGAGGCAGAGCAAGGACGTAATTTTAAATGGTCTCAAGGCCAATGGTTA
ACCTGGTTGCAGAGCCTGACAAAAGTTCAGTTGCTGAAGGCAAGAGGTGTTTCTGGAAAAAAACTATTTCTATAGGTGACGGCGCTCCTGATGG
TGGTGATGGTGGTGGTGGTGCTGCTGCTGAGACTGATGGTGCTGGTGATGATGATGGTGGTGGTGATGTTGAGGATCCTGATGGTGGTGGCGAT
CATGGTACTGATGGTGATAACGCTGGCAACCATGGTCATGGTGCTGATAGTGATGGCTGGTGTTTGTAGTGTGATTTTGGGTGCCAGAGTCAAG
GGTGACATTCTTGTGGGCTCTTGTCTCTTTCCCATGCTATTAACCTCCTCTAGGTTATGAAGACAGGTCGTTTCCCATTTCAAGGCCCTGGGCA
GTGTGACTGATGGGTCTCAGAGTGGGGATCTGCCATCCTCACTGCTGCTCGCTGTGAGGCAGGTGGCATCTCTGTGCCGTCCTGCGAGCCACCG
TGCTGCCTGGTTTCAGGACAGGCTCGGTGGGGCTCAGGGAGAAGCCCTGGGAGCTGTCACGAAGGTAGAGGCAGGTGCTCACCAAGTAAGAAAA
ACACAAATAGACGTTCGCCCCTCTCTCTAGTGCTGGCCTGTCCATGTGTTGGGGTCCTTTTAGATCTGGCTGTGGATGGCGAGCTTAGCTTGTA
CAGCTTCCACAAGCATTTGTCTTCTCGACTGCCCTCAAGGCTTCTGTAACCCACTCAGGAGGCCCGGGGTCACGGGCTGAGCCTTTGGCCTTGT
CAGCCCCGGGCTGTGGCCCTGAGAATCTGCTGACCAATGCATTCTGGGGCCACACTGGCCGGGCCAGACCTGGGGCCTTCTGAGTGAGAGGTAA
AGGCAGGCTGTGGGGAGGAAGTGCCCTTGAGCCTTGCACAGCTGCCTCAGCACAAGTGAGGGAAGCGAGGTGCCCTCAGCCACAGCTGGGAGCT
CTTCTTCCAGAACCCAGGCCACATGCCCCGTCTCTGTAGATACGCTGCAGCCGGGCTTCCTGACTGCAGAGATGACCTGGGGTGAGATGGGTGA
GGCAGGGGGACCACAGGGAGTAAGGGAGACCATGGCACTGTGCAGAGCCTTGACTCTGAGGTCTAACCCATGGTAGCTGCCTCCCTGTGGTAAG
CAGTGTTATAGGTCTTGAGACCAGGCTAGGATAGACTTCCTATTTATCAGAAAAACCCAGACCTCTGGCTTTTAAATTTTATTTTATTCATTGA
TTTATTGATTTATTTTTAGAACAATCTTGAGAGATGTGTAGCTCGAGATGGTCATGAAGTCACAATCCTCCTGCCTCGGCCTCGAGTGCTCTGG
CATGAGGCACCCCTCTCTGTATTTGCCACCCACGCCAAGGCTTTCTCAGAGGTGCTCTGCTGTCAGACCTCTTGAGGCTGCCAATTTGTTCCCA
GCCTGTGTGCATCTAAGGAATGGGTCAAACAGCAAGGCTTTTTTTGGTGTGTCAGCACTTCAGGGTGAGGCAGAGCCAGCCTGCTGTGGAGAAA
AATGCCAGGCTCCCCCAGCAGACAGCTGGAGTGGCAGGGCTTGCCCTTTCACCTCTTCAGCCTGCAAAATGGCTGCGGTGACCAGCGATGTACC
ACAGGGGCCAGATGTATGGATATGCCCAACCCCCGCAGAACCAGGGATTTCAGTACGTAGTCTCAGGGGTCTCACCTGTGGACCATATTTTGTC
CCGTTTCCCTGGCTCCTGTGGTCTCACAAGAGACCCCCCCACAAAGCTGGCGCTCTTGTCATCGCTACCTTGCTGATGAAGAGCAGGCACAGAG
AGGTGGAGCCGCTTGCCTCAAGTCACACAGCTGTGAGGAAGCAGTGCTAGAATTCAGTACGAAGGCGATGCAGATGGCGATGCTGATGATGCTG
ATAGCGATTTTAACGGTGGGCCTGGGAGTGAATGTGGGGCGAGCTGCTGGTGTCACCCACACAGGCCCACCCAGGCAGTGGTGCAGTTGGTGAC
TGCACAGCTTTAGGACACCATGAGTTTGGGGAGCTATCAACTTGTGTGGCTCCAGAGAGAAGCCTGGATTGGCCTGAGGTGGCGAGGCGAAGCT
TAGGATGCACAAGGCTGCCGACAGTGGAGAATTCAGTTTATAGCAGTTACAAAGCTTCTCCAACAACAAGCCTCTAGCTCTGGGCTCCTGGTGT
CCGAGCTTCCCTCCCAGGAGTCCCGCCTCTATGATTCCCTCCCTGGAGCCCCGCCTCCACACTAGCTGTTAGTCAGGGGCCGCTGGGCCCTCTG
CCTTGTGTTTTTCTCATCTTTCCACTTTGTAACCTTATCTGGAATCCGGTTGTCCTTGCCTGCAAGAGGACGTCAGTACCTTCAGTACCGCCTG
CTAGACAGGAAGCCAGAGGCCGGAAGAAAAGGAAAGTCAACGAAAGCCAAATACTGGGTTGGCACGTTTCCAGCCTCAGGGTTCAAGTTCAAAG
TCTCCCTCCCACCCAAAAAAGATCGTTGGGATTATTATGGTCGGTTGGTGCCAGACTGAAGCTTTTCTCTCTTTTTAAAACTTTTTTGTTTTTT
GTTTTGTTTTTGTTTTTCGATTGATGGGTTTACGTTGTGGCACTGGGGATGAAGCCTAGGACTTGAGTGCGTGAGGGAGCGCTTCCCCTCCGAC
CTGCACCCCAGCTCCTGAGGTGACAGAGAGAAGGGTTGGCTAGGATGTAGCCTATGGTCACAGCTCCTGCCTGTGCTGCTCATAGGTGGAGAGA
AGGCTTCTAGGTCTTTCTGGAGGACAGGCTGTGTGTAGGATTGTACAGAGGCTTTCGATCTGAGTCCAGGTCACAGTGTTTGTGATAATGTTGA
TACCTTCACAGGTGGGTTTCTTCCCATCTCTAATGGGGGCTACCGAGTGCAGGGTGACTCGGGAACCAGAGCCATAGGCAGCTCATGAGAATTA
CATTGGCATGCTTGTATGTCTTGAGCTTGGTACAGGCCTTCCTAGATCTCTCTGTGTTTGCGCATGTGCATGCACATGTGCACATGTATGCATG
AATGCATGTTCACACCTGTGCTCATGTGTGTGGAGATGTGTGTGTATATGAATATGTGTTCATATGTGTGTAGGTTCATGTGTATGTGAATGCG
TGTTCACACGTATGTACACGTGGAAGCCAGAGGCAAGTCTCAGGTATCATTTCTTGGGCATTGCCCGTCTTATTTTTTGAGACAGGGTCTCCTG
CTGGCTTGGATCTGACCAACTAGGTGAGACTGGCTGGCCAGCCAGCCTCCGGGGTCAGCCTTTCTCTGCCTCTCATGGGCTGAGATTACCAGCA
AACAGCACTTGGAATGAGGAATAGACTCTGAGCCTTTAGGGGAGGCACTGGGGCTCCCCATCTTCTAGGCCTGATGGCCCTGTGCTCTTGGCTC
CCACAGCTTTCTCCCAGATGGTCATCAGCTTCTACTACGGGGGCAAGCTGGTGGGCCAGGCCACCACCACCTGCCTTGAAGGCTGCCGTCTCTC
CCTGAGCCAGCCGGGGCTGCCTAAGTTGTATGGGCCGGATGGCCTGGAACCCGTGTGCTTTCCGACGGCCGACACCATCCCCAGTGAGCGGCAG
AGGCAGGTGACCCGGAAGCTGTTTGGGCACCTGGAACGTGGCGTGCTACTGCACAGCAACCGCAAGGGCGTGTTCGTGAAGCGGCTGTGCCAGG
GCCGCGTGTTCTGCAGCGGCAACGCGGTGGTGTGCAAGGGCAGGCCCAACAAGCTGGAGCGGGACGAGGTGGTGCAGGTCTTTGACACCAACCA
GTTCATCCGAGGTCAGTACCAAGTCACCTCGCTGCCACCCACTGTCCCTGCCTTAGAGGTCACACCCACCTCCTTCCTTGTGCCTCCTGTATCA
TGGGCCAAGTGCACTCTTGCATGGCTTTGGCAGTGGTATAGCTGGGGGAACCCTTGGAAGCCTAGCCTGCCTGTGCTTTCTGAGCGCCTGGGCA
ACCCTTTCCTCACCTCCCCCTCGGTATAAGGACAGCAGAGTTGGAGCCTTTCCTTTTGGAGTTGACGAAGCTGATGGTTGTTCCTAGCCTGGGA
ACGGCTTAGCAATGCAGACTCCAGGGCCTCAGCCTGCCCAGGCTCGGCTTCTTTACCCTGACAGGGTCCCTAGGGGATGTGGCATGATTTAGTG
GGCAGGAGGATCATGGAGAGAGAGTTCCAGGCCAGCCTGCACTGCACAGTGAGATCCTGTCTCCCCAAAACAAATCCAGACCAACAAAAAGTTC
CCCAAATCGCCTAGCAGTATTCGGGAGTGACAAATCCCATGGGTCCTTGGTTGGCTTCTAGCTAAGCAGCCTTGTATCCTAGGCCCCTTTCCTT
GGCTGTTGGGGGATCTGTGACTTCCTTCCAAGTTCTGTACCCCAGCCCTGGGCCCTGGCTACACACAGCTCAGCGGTTCACTGGTGTCTGTGTT
CTTCCAGAGCTGCAGCAATTCTACGCCACCCAGAGCCGCCTACCTGACAGCAGGGTGGTCCTGTGCTTCGGGGAGGAGTTTCCGGACACTGTGC
CCTTGCGCTCCAAACTCATTCTGGTGCAGGTGAGAGCCGGCAGCAGTGTCTGCCTGTGCCCAGGGTGAGAAGAGACAGCAAGGACTGGGGGCTC
CACCTGGGCAGAGGTCGGGGACTAGATCATACGTGGCTCTTGTCACCTCTGTGATGGCTTCCCACGTTGGGACTCTCTTCATTTGTGTTTCCTT
CTTCCCCTCTCTGCTTGCTCCTACTTTCCCTCCCTCTGTTCTCCTGTTCCTCCCTCTTCAGGAAATTTCTGCTTGTCCAGTGGCCCTAAGATCT
AAGTGACAGATCACGGTCTATTTGACTATGGTCTGAACGGTGGCACTCTTGTAGATGTAATGGTGACAGTCTGAGGAGCAGCCAGCCTGGTTGG
AGCAGTTCTGGGAGAACACTCTTAGCCATGTGCAGGAACGCTGCCCACCTGGCTCCATCTTGTCTCGGAGGGGATTGGAGGACCTGTGGTGGAG
GATGGCCCCAGTTCCCCTCCACCTTGATTCTGACTTTGCACATGGCCCTTGCAGGTAGAGCAGCTGTATGCCAGGCAGCTGGTGGAGGAAGCGG
GCAAGAGCTGCGGTGCTGGCTCCCTGATGCCAGCCCTGGAGGAGCCCCAGCCGGACCAGGCTTTCCGCATGTTTCCGGATATCTGTACCTCACA
CCAGAGACCCTTTTTTAGAGAAAATCAACAGATCACCGTCTAAGCCTCAGTCCGGGCACCCCACCTCGCCTGAGCTCAAGCTTCAAGAGTCTGT
GACTAAGAGAATTCCGAAAGGATGTGGAGCCCTCTGACTGGGGTGGGCGGGTGTCCTCCAAGGGGCCTCCGGAAGCCCACAGAGGGATGCGCTC
CTGCTCAGGCAGGTGTCAGAAGCTTGCAGGGGCTGTGGCCGCAACCTGTGATTAAAGCATTCCTTTCCTGCGTTTCCCCCTTCACCACTAATGG
CTGGCCTTTCTGTGTGCTGAGGTCTTTCGACAGTTCAAATCATCTGGTGGCAGCAGACTCGCCTTTGCCCTTCTGCGGCCGAGGGCGGAGATTT
ATGACTTTCTCTGCTTGGTTGGAGAAGAAGAATCTTTACTATTCAGCTTCTTTTCTTTTTGGCCAGAACTCTGAAAAAAAAAAAAACTCTTTTT
AAGACAATATTTGTATTCTCACAGGCTCAGCTGTCAATCACTTGAGACCTTCCCTGTAAAGTGGGGCAGATTTTAAATATGGGTGTAGATACTG
CTTGCAGCCTTCGCAGGAATTTTGGTTGTGGTTCATTGATTCACACAGACTCTGTGTCAGCTGACAGGGCTGTGTGGGGCATCAAAGGAGGACC
AGGCACTGTGGAGAAGACCCATTCACTGGCATCTCACCCTTCCTTGTCCAGCTCCATACCCAGTCCTAAGACCCAGTGAAAAGCCACGTCCAAA
CTGTGCTCTGGGCTCATCAGTGCCCACCCACGTACCAGGGAAAGGCACACACCCTACCCAGTGGGCACAGAGCGGAATGTCCCCCTACCGCACC
ATTTGCGCCCCCAATCTGGCTGTCCAACCTAGTTTGTAAGTAATCTAAATCAGTGACTATAGCCCCGCCTAAGGGACACTTCCCGGAGGAGGGA
GCCGCTGAAAAGGAGTTAGTTTGAGGGTCAGTACACAACAGGGGCAGAAAGCCAAGCAGATGTGGGGGCAGGGAGAGTCATCATCTGCTTTTGT
CTGAGAGAAGGAGAGCTTCTCCGTTTGTTCAACTTTGTAACAAGCTGGGTTACATGCTCCACGCAGCTAGAGAAGCCTAGGTGCTCTGCATTCC
CTGGGGAACTGCAGGAAAGCCTTACCTGCTGACTGTTGCTCTGGGGAAAAGCCTGAGGGTCCAGAGCAGCTACAAGCTACAGGCCATACCTTAC
AACCTGAAAAGCTAAGGACCACGGTGACCTTCCCGGCTACTGTGTGAAGGTGCTGGGTGGGGCCTGCTCAACAGACAGGGTCGACAGAGTGTGT
GATACATGCAAACAGAATCCTTGGAGTGTGTGATACATGCAAACAGAATCCTGGGCCCCTGCTTCTCCCCCTCAGTCAAAGCAGGAGTGTCCCT
TCCGAAGCCAGGACAACCTGTTCACAAGGCCCCTTGTCACATGTCACCTTCCACCTGCCTCAAGGAGTGCTAGTGTCCAAATATTTATTTTTGT
ATTCTCTTAAGAAGTATTGATTTCATCCTTTATTAAAAAAAGTTGCTCTTTCACAAAGTAATGTTTGTTTCTTTTTTTTAAAAAATCACTTTTG
AATATAGTTTTCATTAGATTCCAGGATTTTTTTTTCTTTTTAAATTTTGATGTTAAAAAACGAGACCAGAGGTCTGGAGAGATGGCTCAGTGGT
TAAGAGCACTGACTGCTCTTCCAGAGGTCCTGAGTTTAATTCCCAGCAACCACATGGTGGCTCACAACCATCTGTAATGGGATCTGATGCCCTC
TTCTGGGATGTCTGAAGACAGCGACGGTGTACTTATCTACATAAAGTAAATTTATTTTTAAAAAGTCTTTAAAAAAAATGAGACCAGATCTGAC
TCTGCAGCCCAGGCTTTGAACTCATTGCAATCTTCCTGCCTCAGCCTCCTTAGCAAGGGGAGGAGAGGTGTGAGTCACTGTGAATCCTTAAAGG
AATCTTTAAAATAAATCTTTAATTTACTTTTAAGAAATAATGGATTGAGATTTTCAAGGATTTAGGGATTTTTTTTTCTTCTCTTAAGACAATA
GCAGAAAGCTCTGGAGAATAACAGACACAGGAGAGCACAGAGGGCGGGCAATCCACGCTGTTCTACCAGATGTAGATTAGGGTTTCAGTGAGCC
CAGGCCTCGTGCAGCATGGGAGGGCATGGAGGAAGGAGACTATGCTGACAGCACCCTTCTGGAAGCTTCTAGAAGCATTAGTGGGACGGTCACC
AGTCATAGCGCTGAGGTGTGATCTTCCATTCGTGCACACATACTTATGCCAGACACCGGCTCAGAATGACCCTCACTGGTGCTATAACCAGCCC
AGGGGCCATTTCAGTAGCAACTGGGAACAAAGGCCCTCCCCACCCCCACACCGCAGGTGGGCACAGACTGCTAGTTCTGCTGTCTCTGAGGCAG
TGTGCACTGTCTCTGCTCTCATGGGATCTCAGAGACAGGGCCAGTTAGTCCCAGCTTACCCATGTGGAAAGTGCGGGAGAGGGCCATGTTTGGC
TTGAGTGAGGCCTCCCTTGTTCCGGGAGTGCAGTTAGAGCTTCTGCAAGATCCTTGGCCCAGAGGACAGCTGACTGCAGAGCAAAGTAAAGCAG
CCATTTCTGCAGGGAGTCCCGTTGGCTTCAGAGGGCTCCAACGGGGGTCTTGTGTATAAACAGCTCCTCTCTGGATGTGCAAAGTGAATCCAGT
CCTGTTTGTGTCTCTCAGAGTTCAACCTCTGGGCCAAGAGTGGATGCTGTCTCCCTGAAGTGCGGGAGGGAATTTGGCATCTCTGAAGGCTGGG
GCCTCCTGGGACAGGACTTGGGTCCTCAAAGGAATGTGACTTTCTCTGGGTGGAGACTGTTTGGTCCTGAAGGAAAGGCCAGCTACCTAACCAC
CACTCTTGGGATCATATAAAACCCTTATTAGGTTAGTGTGTGTTAACATGACTCTGAGTGGCTACTTAGTTCCCTAATGTCTGCAAGGTCCACC
TGAGCAGAACAAACTTTCTGCTTCCCTTCATACCAGTCACAGAAGACAAGGACCCTAGAGACATCGAGGGAAGAGCAGGGTGGGGTGAGGCAAG
TGCCCTGGGGTGCCTGCTGTGTGAGGTATTGGCTGGGCCGGTGAAAGACAAGGGTTGATTGTGTGGCCAGAGGACAGGCTAGGGTTCCACTCAT
CATGCTTCTGTCTCCCATTTCCCTCACCATCGAGAGGGATTTGATACCACACACAAAGAGGCTTAACAGGCCCGAAGAGCACAGAGCAGAGCCT
GCCGCTCAGAAAATACCCAAAATAACCGTATTTCTGTTTACCACTCATCTGTAATTAAGCCAGGAGTGATTAGAGAGCTGAACTATAAATATTA
AATAGTCCTTGCTGACACGCTACAACGCCAATGGTTAAGTAAGTGTGTGTGTGTGTGTGTGTGTGTGTGTGTGTCAATCAACTATGTCAATAAT
GTAATTGTTAATAGGGTGTGGTGGTGCACTCCTGTAAGCCAAGCATTCCAGAAGCTGAGGCAGGAGGATGGTGAGTTCCAGACCATGCTGGGCT
ACACAGTGAAACACTGTCTCAAGAAACCAAGCTGCAACAATAGTGGAATTAGCACTCAATTGTTTGGAGTGATAAAGAAACGTATGACTGTGCA
GAGTTATATGTTTTCCCTTTATTTATGGACACCGATTTAATTGAATATGATATTAATGTGGTCATTTCTGAGCCTTCCATTGTAAACAGTAATG
AATTTATTAAACTTCTCTCATTAGGTTCCTTACACAGAGTCTGTGCTAGCCTAGTGGAAGGGCTGCCTTGCTGACTCCCCGATCCTGGCTTCCC
TTCCGGACCAGAGCCTGGAGTTCTGTAGCTTTCTCTTTTACTATTATTATTTTGTTTGTTTGTTTCTGAGAAAGAATCTTAATCTATATTCCAG
GCTGACCTCAAACTTGGTTTTGATGCTCACGGCAGGTGGTGGGACAGGCCAGTGAGGCGTTGCCTGTGATCCACCCACCGGGAGCCCTGTGATG
GGAAGAAGGGATCAGTGGTTGGCTCAGGGCTTGGGAGAGACTTGAGATCCCCTCATGAAGGTGATCCCATGTTGCTTTAGGCAGTCCTACGGAC
TCGCACATCCTGCAGGAGGAGTCGCTGTGAGCATCCCTTAGAGGACATCAAAATAGTGTGCTTTTGGTTTAGCCTTTCCTATGCGGTGGGAGGG
GAAGGGGCGGGAGAGGGAAGGAAGGGCAGAATCTGGGCTCTAGGATCCCTTGTGGTGAGGTTTGGGGAAGGGAATAGGCTTTCGTGGATGCTGC
CCGGTGAGCAGGGTCCTCCTGTCCTGCCCCCCCATCCCGTTAGCATGTGCAAGTGGAGGACTGAGCTCTGAGTGACCCTGGCTTGGAGCTGTGT
TGCAGATTGGGACTGAACTCAATATATGTTGGCATTATGGGTCATACCTTTGTGCTGAGGGCTATCCCTACTATCCCCTACTTTGTGCTGAGGG
CTATCCCTACTATCCCCTACTTTGCTTTTGTCTGAAGTATATAGAAAAAAAACTTAAAAAATTGCCCAACCCCAAAAGGGGACATGGGAGGTAT
CCCCTTAGCAGGTCCTTAGCCAGCTTTGGGGGAGAAAAACCAGTGTCTCAGCCACCCTGGATCCCCTTTTCCTTCCTGTCCGTGGCTTCTGCAA
GGGTCTCTCAGCCTCTAGTGCCATGAGCGGGGCATTGGTCAATGCAGGGTAGAGATCCCGAGGCCAGAATAAAGTGACTCTTGCCAAGGCCTCT
GAGAACAAGTCAAACATCTGATGATGGGAGAGTCTTCTTGATTATCCATGCAAGCCAGTGTCACAGGGTTGAGTCAGAACTATGAAGAAGGGAC
ACGTGGGTCAGAGCCAGAGAAGACACGTGACACGGCAGGACAGGATTTAGTTGGGCTGAGATGACTCTTGAAGCTGAGGACTGGCAGGGAAACA
CAGGAGGTTCTAGAAGGAACCACCACCCCAATCTGTAGTCCTGACCTTGGGGACTGTCAGGGAACGGGTGTGTGCTGTTTTTAAGTCACTGCTA
CAGCACTGTCCACAGCCATAGGACAGTGACACAGAATGTTGACCTTCAGAGGAGAAACCCAGGACTCCCGGGATGGGAGTTAGGGATCCTGGGT
TGGATGGCGGGTCACATCATCCCCTTTTCTCCGGACTCAACTTCCTCCCAGCTGTCTCTCTTTCATCAGCATCCTGTCCTTCTCACATGGGAGC
CACACTGCCCCCCAGTACTTGGGGACACTGGGGTGGAAACCCCAAGGTTGAAAATAGCCCTGTATTATTCTTGGTTAAGTTTCTGCTCTTTCCC
CGAGGCAGCCGGCTGAGAAAGCCACTTCCTTCTGCCAGCCTGCCGTCCATACTGGCATCTGTCTTCTGTCCTGCTGGGCTTTCAGCCACCTGGG
GCTGGCCCAGAGGTGGGCTTCTGTGGAAAGATTTTCCCTGTGTTCCTGCCCTGACTTGTGGCAGAAGTTTTGGNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNCAAGC
TTGACCTTAAAGGACCGGTTGCCACTGAGAGATGTAAATGACGCCATAGGACACAGATCGCCTTAGGTGTGGGAGGAGAATGACACTTTTAAAG
AGGGGCTTCCCGAAGGCAAAGCTTCAGATGATGGCTTGTCTCGCTCACCACAGAGCAGGCCAGAGATGTTTGTCCAGTGAAATGGATGTGGATG
TGTAAGCGAGGAGCCGCTGGGTCTGATGGGACTTGAAGGTTCTAGAACATATGGTCACCCTCTCTGCTAAGTGTGACCCATGTGCTGATCCCTT
GAAATCATTCTCCAGGTGGATTACAGTGCCTCGAGTGTCTTCTGGACTAGAAGACAACCCGGTGGGGACAGCGATCCATTCCAGCATGCTGGAT
TCTACCCACGATCCTGAGTGGCCCCAGTGATATCCAGGGCAGGTCCCCTCACCCATGAGTTGCATGCCTCAGCCACGTCCAGGGGCAGGTCACC
TCAGTCATGATCAGGAAGACTCTGTGACAGGTGGTGGAACCCCCATCCCATCTCCCTCCACTTCAAGAACATCACTCTCACTATTCAGATAGAG
AAATTAAGACCCAGAGGTGAGCTATAAATGGCAGAGCCGCTTGTAGTGGTCATGAGGTGGCCCAGTGCTTAGTGGAAGATGGGAGGTCAGCAGC
CTGACAGGTTCCTTACCACAGAACCCTAACTGTGTCAGGCACTCTGTCTCCCTCTGTGTGTTTCAGGGGAGGGTCCCATTCTCACCCAGCTATG
GGCCTTGACATCTCTGAGGCAATGGTTCTTCCAGAAGGGTCTGTGACTGGATAACATCCAGGGAATACAAGGGAAGGCTCCACTGGGCTTCTGG
GAGGTTCTTCCAGCCCTTCTTAGCCAATGAACAGGTACGAGGACTCTCCAGCCTTGAGTCTCCATCTTTACTGCATCACACGGGAAACTCCTTC
TTCTTACCTTGCTTGGCCTCCTGTGATGTGGGACTCTCCTATATGTCATGGATAGGAGCAAAGGGTGAGGCATATGAAGCAAGGTGAGTCCGAG
AGACCACCTAGCCGGGAGAGGTTGGGTGTCACCCAGCTCTCTTGCAGGTCTGTTTCAGACACTGATACAGAACCTAGTGTTCTGGAGCAGAACT
AGACCAACCTCACACCTCGAGCATTCCTGGATGGGTGCTGGGTGGACTTCCTGGAATGCTTAGTTCATATCAGAAAAGAGTGGGGATTTTTTTT
TTGGCCGCTGTGTTGTCTTGTCCACGAGACCCTTATCATGGGCTGCTTTGCCATCCTGGTTCACCTCCTTTAGCCGCTCAATAATGTCCTGAGA
TGGTTGTCTCTGTTGACACCAAGGAAGCAGAGGAACAGCAGGGTCATAACGACTTGCTCAGGGCTGATTGGTGGTGTTGTCTGAAGCCAGTCTC
CTTATCCACCATTGCTGTCCTCACAGAGGTTCACTGCAGAGCCCACGAAGGTTACAACCTGAGGGTTACAACAAACATTGGACTCCTTGTTGCT
TGGAGGAGATGACCTTGTTACAAAGTTGCTTGCTGGAGTGACAGTTGACAGCTAAGTCTCCGCCTCTGTGTTTTTCTGGAAATTAGAGGCTAAA
GTCCAGGACCACATATCGTGAATAGAATCTCCCACCACTCAACTCAAGCCATGATGGACCCAGCCCGGGGTGGCCTCTGCTCCTGACACGGATA
ACCCCTGCAGGGAACTAACCAACCTTAGCACACTTTTGTTAATGAAATCCCACACTTTTCTGGTATTTATTAAATGTCTCCTCTCCCTTTTACC
TATCTTGGGACCCATCATGATCCCATATCCTAGGGTAGTCACATCTCTTTGACTGTGACTCCTTCAGACTGCCTGGCTTGCATAGCCTTGACTA
TTTTGAGGTGTCCTACAGAGGCTCCCCCCAGCTGGGACTCTGTAGTCTTTTCCTCCTGTTTAACGGAGGATACTGAGACCAGACATGGAGATGC
ATGCCTTTAATCACAGCATTCAGGAGGCAGAGGCAGGCAAGTCTCTCAGAGTTAGAGGCCAGCCTGGTCTACATAATGACTTTGAGGACAGCCA
GGGCTACACAGTGAGACCCTGTCTCAAAATAAATAAATTAAAAATAAAATAAAATAAAACCACAAGCTGCTGGGATGTCAGGCACACTACACAG
GTGGATTTTTTATGGGTGCTGGTCTTGGTCTCCCAGGAAATTGTTCCCCCTGTTCACAGAGGATTCTCCAAGTCCAAAGGCCAATAACAATGGG
ACAGTTCAGGGCTCAGGAACATGTGCTCTGCCAGGGGATACAGGCTGAGACATCTTTGTGACCCCCACGGGTCCCCCCCAGTTCCTGGGGTCTC
CAGCAGTTCCTACATACTTTACTTTTATTTTCTATTTTTTAAGAATGCTCTTCAGAAAGTCAACCTGAACAGTTTTCCCAAAGGTCCAGAGATG
ACACGCCTGCGACACTCTGGATCCCCAGCCTGGTTTCTCTCTGAGCAGCTTCTCCTTCCTGCCACCTCAGCCACGACAGCTTCCTTTCGCTTTT
CTCAGGATCCACACACCCTTCCAGTGATCTCAGAGCCAGTGCACCCACCACTTCCTCTGCCTGGAATCTGCTTCCTTCCCTCGTAAGCTGGTTG
ACAGCTTCCTCCTTCAGACTCCTCTGCCAACCTCCAGAATAAAATAAACAACTCCCTTTGCACTGTGGGAGCTCAGTACAAAGGGAGGCTCCTC
TATAGTACACTCAAGGAAGGCAAGACTCCTTCCATTTGTGTTCCCTGGTGTATCTTCAGGCCTGCAGCCCAGAGCCAGGTGGATGATAAAGGGT
CTAGTACTCTTGTGACTCCATCCATCCACCGATCAATCCACCTGTCACTTGCTCTTGAATTCACTTAGCACTTATCCACCCAAACATCTATCTA
TTCATCCACCCATTCATTTATCCACTTGATAATGCATCTATTCATCCACTCAAATATCCACNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNTGTAAGGTGGGGGGGAATGCTGGCCTGCAGTGTAAGGTAG
GGGGGAGTGCTGGCCTGCAGTGTGAGGTGGGGGGAGTGCTGGCCTGCAGTGTAAGGTGTGTGTGGGAGAGAGTGCTAGCCTGCAGTGTAAGGTG
TGTGTGGGGGAGAGTGCTGGCCTGCAGTATAAGAGGGGGGAGTGTTGGCTGGCCTGCAGTGTCAGGTGTGGGGGAGTGCTGGCCTGCAGTGTAA
GGTGGGGGGGGAATGCTGGCCTGCAGTGTAAGGTGGGGGGGAATGCTGGCCTGCAGTGTAAGGTAGGGGGGAGTGCTGGCCTACAGTGTGAGGT
GTGTGTGTGTGTGTGTTGCTTTCAGTGTAAGGTGGGGGTGGTGCTGGCCTGCAGTGTGGTGTGGGGGTAGTGCTGGCCTGCAATGTGAGGTGGG
GGTAGTGCTGGTCTGCAGTGTGAGGTGTGTGGGGGTGCTAGTCTGCAGTGTGAGATGGGGTGGGGTTTGCTGGTCTGCAGTGTGAGGTGTGTGT
GTATGTGGAGGGGGAGTTCCTGGCCTTCAGTGTGAGGTGTGTGTGTGGGGTGCTGGCCTGCAGTGGAAGGGAGTGGGAATGGGGCTTGGACACT
ACAGGATGCCCAAGGGAGAAGATGCCTTTTGAACAGGATGATTGTGGTGCGATGAAATACAGGCAGAGAGCAGGGATGATGTCCAGTCGTGAAA
TGGCTCTGCGGGAGCCCCAGAGATGGCTGAGAGTGGCCAAATGTCCCAGGATGTGACTGCTGTGAGTGCTGCACTGGTTATAGGGGGCTTTAGG
CTCAGGAGGCCAGAGCCATGGCTGTGACCACTGCCAGGTGTCATGTGGGCGGGGCTGGGATGGAGGGAGAATGGTTTCTGTTGCTCTAGCTGTG
GGAGGGAGCTCTGTGCCCCATGCTCTACCTTCCTCCTCTGTAAAGCCAGGATATCCTGGGACTCCTGTTTCTCAGATCCACGTGAAGGAGAAGG
GAGAGTGCCCACGTGCTGGTCCCCAGTGTGATTTCCCCACATTGATGACCCTTGTCCAGGCAGCTAGGTGGATGCAAGTGACCAGAGAAAGCCA
TAGGTCAAAGGAACAGGGCATCCAGCTGGCATGGGGGGGGGGTACTGTGAACCCCACAGAATCAGCTCTGCCTGGGGAGCTCCCAGGTTCAGAG
TCAACCCCCGTTAGCATCCTGGTGTATGCTGTGGGTTTGGCCAGGTGAAGTCCTTGTCTCTTCTGGGCTTTCTTGTAGATCATCGGTTCCAGAG
TGAACTTGGATGCTCTCTCATGACCATGTCTGCTCCCACCACAGAGCCACTGATCTGGGCTGACAAGATCCAAACCTGCTATCTTCAGTAGCAG
TGTGAGGCAAGGGACTTGCAACTGCTCAGGAACAGACTGTTCAGTTCTGGGGCCCCAGCACCTCATAGCTGGTGTCTCATCCCAACCTCGCCTG
CCTCTTGCCTGGTTACTGCATCTCTGCTAAAGTCGGCTGCAGAGATGGAAGCCTGTGCCATTAGGTTCATGACAAGCTGCTGTCATGTCCTGTG
GGGGCTCATTGCCATGGGTGCAGAAGGCAGTTTTCCCTCCAGACATTTGGCTGTGAATCTGGTCTGCTATAGCAGCTCCTAGCTCATCTCCTCT
GCTTGG
MOUSE SEQUENCE - mRNA
CCGGAGGCGCGGGCAGCGTGGGAACCGGCGGCAGGATGTGTGACCGGAACGGCGGGCGGCGGCTGCGGCAGTGGCTGATCGAACAGATCGACAG
CAGCATGTACCCGGGGCTGATCTGGGAAAATGATGAGAAGACCATGTTCCGTATCCCCTGGAAGCATGCCGGCAAGCAGGATTACAATCAGGAG
GTGGATGCTTCCATCTTCAAGGCCTGGGCAGTTTTTAAAGGGAAGTTTAAAGAGGGAGACAAAGCTGAACCAGCCACGTGGAAGACGAGGTTAC
GCTCTGCTCTGAACAAGAGCCCAGATTTTGAAGAAGTGACTGACCGGTCCCAGCTGGACATTTCTGAGCCATATAAAGTTTACCGAATTGTCCC
CGAGGAAGAACAAAAATGCAAGCTGGGCGTGGCACCTGCAGGCTGCATGAGCGAAGTTCCTGAGATGGAGTGTGGCCGCTCAGAGATTGAGGAG
CTGATCAAGGAACCTTCTGTGGATGAGTACATGGGTATGACCAAGAGGAGCCCATCCCCACCAGAGGCCTGCAGGAGCCAGATCCTCCCTGACT
GGTGGGTCCAGCAGCCCAGTGCAGGCCTGCCACTGGTGACCGGATATGCCGCCTATGACACACACCATTCAGCTTTCTCCCAGATGGTCATCAG
CTTCTACTACGGGGGCAAGCTGGTGGGCCAGGCCACCACCACCTGCCTTGAAGGCTGCCGTCTCTCCCTGAGCCAGCCGGGGCTGCCTAAGTTG
TATGGGCCGGATGGCCTGGAACCCGTGTGCTTTCCGACGGCCGACACCATCCCCAGTGAGCGGCAGAGGCAGGTGACCCGGAAGCTGTTTGGGC
ACCTGGAACGTGGCGTGCTACTGCACAGCAACCGCAAGGGCGTGTTCGTGAAGCGGCTGTGCCAGGGCCGCGTGTTCTGCAGCGGCAACGCGGT
GGTGTGCAAGGGCAGGCCCAACAAGCTGGAGCGGGACGAGGTGGTGCAGGTCTTTGACACCAACCAGTTCATCCGAGAGCTGCAGCAATTCTAC
GCCACCCAGAGCCGCCTACCTGACAGCAGGGTGGTCCTGTGCTTCGGGGAGGAGTTTCCGGACACTGTGCCCTTGCGCTCCAAACTCATTCTGG
TGCAGGTAGAGCAGCTGTATGCCAGGCAGCTGGTGGAGGAAGCGGGCAAGAGCTGCGGTGCTGGCTCCCTGATGCCAGCCCTGGAGGAGCCCCA
GCCGGACCAGGCTTTCCGCATGTTTCCGGATATCTGTACCTCACACCAGAGACCCTTTTTTAGAGAAAATCAACAGATCACCGTCTAAGCCTCA
GTCCGGGCACCCCACCTCGCCTGAGCTCAAGCTTCAAGAGTCTGTGACTAAGAGAATTCCGAAAGGATGTGGAGCCCTCTGACTGGGGTGGGCG
GGTGTCCTCCAAGGGGCCTCCGGAAGCCCACAGAGGGATGCGCTCCTGCTCAGGCAGGTGTCAGAAGCTTGCAGGGGCTGTGGCCGCAACCTGT
GATTAAAGCATTCCTTTCCTGCGTTTCCCCCTTCACCACTAATGGCTGGCCTTTCTGTGTGCTGAGGTCTTTCGACAGTTCAAATCATCTGGTG
GCAGCAGACTCGCCTTTGCCCTTCTGCGGCCGAGGGCGGAGATTTATGACTTTCTCTGCTTGGTTGGAGAAGAAGAATCTTTACTATTCAGCTT
CTTTTCTTTTTGGCCAGAACTCTGAAAAAAAAAAAAACTCTTTTTAAGACAATATTTGTATTCTCACAGGCTCAGCTGTCAATCACTTGAGACC
TTCCCTGTAAAGTGGGGCAGATTTTAAATATGGGTGTAGATACTGCTTGCAGCCTTCGCAGGAATTTTGGTTGTGGTTCATTGATTCACACAGA
CTCTGTGTCAGCTGACAGGGCTGTGTGGGGCATCAAAGGAGGACCAGGCACTGTGGAGAAGACCCATTCACTGGCATCTCACCCTTCCTTGTCC
AGCTCCATACCCAGTCCTAAGACCCAGTGAAAAGCCACGTCCAAACTGTGCTCTGGGCTCATCAGTGCCCACCCACGTACCAGGGAAAGGCACA
CACCCTACCCAGTGGGCACAGAGCGGAATGTCCCCCTACCGCACCATTTGCGCCCCCAATCTGGCTGTCCAACCTAGTTTGTAAGTAATCTAAA
TCAGTGACTATAGCCCCGCCTAAGGGACACTTCCCGGAGGAGGGAGCCGCTGAAATGGAGTTAGTTTGAGGGTCAGTACACAACAGGGGCAGAA
AGCCAAGCAGATGTGGGGGCAGGGAGAGTCATCATCTGCTTTTGTCTGAGAGAAGGAGAGCTTCTCCGTTTGTTCAACTTTGTAACAAGCTGGG
TTACATGCTCCACGCAGCTAGAGAAGCCTAGGTGCTCTGCATTCCCTGGGGAACTGCAGGAAAGCCTTACCTGCTGACTGTTGCTCTGGGGAAA
AGCCTGAGGGTCCAGAGCAGCTACAAGCTACAGGCCATACCTTACAACCTGAAAAGCTAAGGACCACGGTGACCTTCCCGGCTACTGTGTGAAG
GTGCTGGGTGGGGCCTGCTCAACAGACAGGGTCGACAGAGTGTGTGATACATGCAAACAGAATCCTTGGAGTGTGTGATACATGCAAACAGAAT
CCTGGGCCCCTGCTTCTCCCCCTCAGTCAAAGCAGGAGTGTCCCTTCCGAAGCCAGGACAACCTGTTCACAAGGCCCCTTGTCACATGTCACCT
TCCACCTGCCTCAAGGAGTGCTAGTGTCCAAATATTTATTTTTGTATTCTCTTAAGAAGTATTGATTTCATCCTTTATTAAAAAAAGTTGCTCT
TTCACAAAAAAAAAAAAAAAAAAAAAAAAAAAAAAAAAAAAAAAAAAAAAAAAAAAAA
MOUSE SEQUENCE - CODING
ATGTGTGACCGGAACGGCGGGCGGCGGCTGCGGCAGTGGCTGATCGAACAGATCGACAGCAGCATGTACCCGGGGCTGATCTGGGAAAATGATG
AGAAGACCATGTTCCGTATCCCCTGGAAGCATGCCGGCAAGCAGGATTACAATCAGGAGGTGGATGCTTCCATCTTCAAGGCCTGGGCAGTTTT
TAAAGGGAAGTTTAAAGAGGGAGACAAAGCTGAACCAGCCACGTGGAAGACGAGGTTACGCTGTGCTCTGAACAAGAGCCCAGATTTTGAAGAA
GTGACTGACCGGTCCCAGCTGGACATTTCTGAGCCATATAAAGTTTACCGAATTGTCCCCGAGGAAGAACAAAAATGCAAGCTGGGCGTGGCAC
CTGCAGGCTGCATGAGCGAAGTTCCTGAGATGGAGTGTGGCCGCTCAGAGATTGAGGAGCTGATCAAGGAACCTTCTGTGGATGAGTACATGGG
TATGACCAAGAGGAGCCCATCCCCACCAGAGGCCTGCAGGAGCCAGATCCTCCCTGACTGGTGGGTCCAGCAGCCCAGTGCAGGCCTGCCACTG
GTGACCGGATATGCCGCCTATGACACACACCATTCAGCTTTCTCCCAGATGGTCATCAGCTTCTACTACGGGGGCAAGCTGGTGGGCCAGGCCA
CCACCACCTGCCTTGAAGGCTGCCGTCTCTCCCTGAGCCAGCCGGGGCTGCCTAAGTTGTATGGGCCGGATGGCCTGGAACCCGTGTGCTTTCC
GACGGCCGACACCATCCCCAGTGAGCGGCAGAGGCAGGTGACCCGGAAGCTGTTTGGGCACCTGGAACGTGGCGTGCTACTGCACAGCAACCGC
AAGGGCGTGTTCGTGAAGCGGCTGTGCCAGGGCCGCGTGTTCTGCAGCGGCAACGCGGTGGTGTGCAAGGGCAGGCCCAACAAGCTGGAGCGGG
ACGAGGTGGTGCAGGTCTTTGACACCAACCAGTTCATCCGAGAGCTGCAGCAATTCTACGCCACCCAGAGCCGCCTACCTGACAGCAGGGTGGT
CCTGTGCTTCGGGGAGGAGTTTCCGGACACTGTGCCCTTGCGCTCCAAACTCATTCTGGTGCAGGTAGAGCAGCTGTATGCCAGGCAGCTGGTG
GAGGAAGCGGGCAAGAGCTGCGGTGCTGGCTCCCTGATGCCAGCCCTGGAGGAGCCCCAGCCGGACCAGGCTTTCCGCATGTTTCCGGATATCT
GTACCTCACACCAGAGACCCTTTTTTAGAGAAAATCAACAGATCACCGTCTAA
HUMAN SEQUENCE - GENOMIC
CACGAGGTGTGAATTAGACACCCCTGCTCTGGACTGCGCCTCCACTCCCTGAGCAAAGCAATGCAGAGCAGTGGAAAGAGTCTTGGGTTTGAAG
GCAAGAGGCTCCAGCCCCCCGAGCCTCAGTTTCCCCATCTGTCAAGTGAGGATAACCACGCCTGCCCTGCTCTACTGGGCAGGGCAAAGGAGAT
GATTTAAGAGCAAGTATTTTGTAAAAGAGAAAGCAAGCTGCAACTTTAAGTTGCTACTCCTAGTGCCCCTCAAAGCTGCCTAGCAAATTGATTC
TTCTCCGTAGTTTCCTGCATTGAGTTCTAGAACTCAGTGCTGGGAGTCAAAGGGTCCCTGTTGACTCCTGGGCCTGGGGAACCCACTCTGCCTC
GGAACCCTTCCTTGCATCCTTCCTTTGTAGGCAATCATTTGGTGCATCCCAGCCAACCATGCACTTACAGAGAAAGAAACAGCCAAAAGAAATA
GAGAGAAGGGAATTACGACCTGGGGTGCATTTGACTGCTTCAGTTCCGAAAATACTTACTAAAGACCTATTAGGTGGCAATGAAGTTGACCTCA
AATCACAGAACCTTAAAATTGGAAGGGACTTGGAGATTATGGATTAAGTAGTACACGCTCCTGAAATCACACAGCAACACTGCAATCACCTCTA
ACAGGCGGTCTAGCATGTGTTCCCTTCATGCTTCCAGGGACAGGTGGCTCCCACGTCACCAGGCACCTCATTCCACTTGTAGAAAGATTGTTAA
AGCACAGCCCATTATTAAGTGGACTGAGGTAAGCATGGGGGCAAATTCTGCCCACTGACACACAACTACCATATGCCACCAAAGTCCGAAATGA
AGCCTGCTTACAAATGAAGACAATTACAAAATCATCCGTCTTGTCTCTAAGTGCCAGGGTAAAAATGGACTTCTAGGATTGCATTTAAAAATAT
CTCTAACCACGCTCAAAGTTTGTGATTCTCCTTAGAGGTAAATTATTGTTGTTGTATTGATATTTTATTGTGCTTCTGCCATTTGTTTCTCACA
GATGCACCTGCTGCACATCTGCAAAGGTAAGCTGGAGATGAACATGGGTGAGCGTTCTGGATTATTTGGCACTGCTTGCTAAGCACATAGATTC
ATGATGCACTGAGTCAGAATCCCTGGAGGCTCCTCCTGGGAACCTACATCTTGATAAGTGTCCCACTGTTGCTTATTTATGCTAAAATTTAAAA
ACACTCATCTATATAATATACAATAATATATATTTTGTAATTGTTGACTTTGTAATTGTCTTCATTTGTAAGCAGGCTTGGAGTCCCATAGGTG
AACCCACCTCCCAGGTTTCAGCAATTCTCATGCCTCAACCTCCTGAGCAGCTGGGATTACAGGTGCCTACCACCAAGCCCAGCTAATTTTTGTA
TTTTTAGTAGAGACAGGGTTTCACCATGTTGGCCAGGCTGGTCTCGAACTCCTGACCTCAAGTGATCCACTGCCTCAGCCTCCCAAAGTGCTGG
GATTACAGGCGTGAGTCACCATGCCTGGCCCAGAATTTATATTTTTCACAAACCAGTATTTCCTGGCAATAGCCCTAAATGTTCAGGATATTTT
TGATCTATGCTTAAGAAACCTTTTACTCTTAAGTTTTTTTTTCCTCCCCTGGGACTGTTTCAAGTTCTTCCGTTAGTAATAAATGTGAATAAAA
ATTTATACCTGAAAGGCAAAACGTTATTGAATGCTTAATGAAATTTTTTCCTTTACATTAAATGTTTACATACTTTGCCACTCCTCGGAGGAGG
GTTATGAATTAAATACCTAATACATTACAGCTGCTTAATAAACAGCCAATAAATGAACGCCAATGCATTTAAAAGTAATGAGTTTTAGGATATA
AAAAATAGAAATCTCAGGTTTTTATTTGTAATCCTTCTAGAGTAAAGAAACAGATTGCGGCAATATTAGCAATTCGTAAATCACCATCCAAACC
CACCATGAACAGATAGAGCAAACTGACTCAGTGAAAGAGAAATGACTGGAAGCAAAATGGGGTCAAAGTTCATGTCTTTAGGGCAGATACATTT
TACCCAGTAATATTTCTCTTCCCAAGTCCCCTGAGTCGGTCCCAACTTACTGCCTTTTAGAGATCTCACTTTTATTGTAAGATAGCTTCTTACA
CGAAAACATTTACCACTTGTGCACGTATATATATATATATCTATACATATGCACACACATCCTTTGTATTCAACTGCCTGATAAACTGTAGTCA
TTTGTTCTGTGTTGTGGAGACTGATAAGTGAATCTTTTCCTTATCTGCCAGGGTAAACTGCCAACTTTCAAATACTCTTTGGTTTTCCCAGGTG
AGTCATGCACCTTCCAGTGTCTGTAGCCCCAGGGCTGCTCATGCATGTGACTGGGTCTTAGTTTTTTGACCTAGAGAAGGCATACCAAAGTCTG
CTTGAGCCAACATCTTTGCCAGGTGCCCTCATTGTGTTTATTTTTGTCATATAAACCTTAAGTGGATGGCAGGATTATGTTTCCACATGTGCAC
ACCTGTTTACAGACCACCCACATCAGGTAGGGCAATCCCTCTGAGGGGCCGCAGCAGATTGTGAGGGGCTTCCTGTTTTCATTCCAACCTTCCT
CTGAAACACTGGTGTGTAGTGGCAACGTTGTTCTTAAGCTTGAGCCCCCCGACCAAGTGAAGCATGAGAAAATGCAGCTTACCTTAGTCACAGG
CAGCTAAGCACAGGTAGTTAATGGCAGGTAAATTAGCCACAGGCACCTTTGGAACCAGCAGCAGGTGCGGCAGACCGGACCCTCCTCGGAACGA
GGAGCATGTGCAGAAGAGTTCGCACGGCAGGCTGGGAGTGCTGCCTCCAGGAAGGCCTGTGCTGGGGTTGAACCTTAGCTGTTGTCCGGGAACT
TGGATTTCAGGGGGGTTCCTGCCACCCTAACGGATAAAAGTGCCTCACTGTGTCCAAACTATTTGTGCAAACAGTGTGGTTTATGCTGAACACC
TGCTTTCCTTCTGGGATCTGGAGTTTTTTGGAACACGCCAGGCAGAAGGTGCCTATGTGGCCAGCACCCCCCATAAAAACCCAGGGCTCTGAGT
CTCTAGTGAGCTTCTCTGGTTGACAGCATCTCAAATGTATTGTCGCAACTTGTTGATGGAGGAATTACGTGCGTTGTCTGGGATTCCACTGGAG
AGGACTCTGGAAGCTTGGGCCTGGTTTCCTCCAGACCTCACCCCGTGTATCTTTTCCTTGTGCTGATTTCGCTCTGCATCTTTTTGCTGCAATC
AACGGTAGCTGTGAGCGCAACTATATGGCAAGTCCTGCGGGCCCTCCTCCCGCGGAATCACTGAGCCTGGGGAGTTATTGGGGCCTCAACTCAG
ACAGGTATAAAAATTAATACACCTCCTAGAGCACAGAGATGAGCAATGTTCTTCCAGCTTTCTTTCCTTTTGCCCTCCCTGAGATTTTTTTTTT
TTTTTGCAATGAGCTTATACCTTGTCTATTTAAGGCATTTTTTAATTTAGTGTTGGGATAAGTAGGGGCACTGAAATAGCATAAGATTTTCCCC
CTTACCTCAAGAATAATTCAGCAAAATAATTCATAATATAGAGCAGGTACCAAATTATGTTATAAAGATTTCAGTATAGCTGGAGTCCAGTGTT
TTTAAAATGTGGGTCAGTGAAGGCTGGAGTAGGAAGGAGGACAAGATTTAAGGTGGATCTTAAGGATGATGCGGTAGGCAGATTAATGGCCCGC
AGAGAGGTCCAAGTCACAGCCCATGAAACCTGGGATTGTGTTAGGCTACATGGCAAAGGGAAATTCATGTTGTAGATGGAATGAAGGTTGCTAA
TCAGCTGACCTTACAATAGGGAGATGATCCAGGTGGACTCAATATAATTATACGGTCCTTAAACGTGGAAAAGGGTGGTGCACCTGCAGTCCCA
GCTACTCAGGAGGCTGTGGTGGGATGATTGTTTGAGACAGGAGTTCGAGGCTGCAGTCAGGCATGATTGCAGCACTGCACTCCAGCCTGGGAGA
CAGAGCAAGCCCTTGTCTCTTAAAAAAAAGAGGGGGCAGGAGAGAAGGGCAGAGTGATGTGATGTGAGAAAGACTCACTCGCCATTCACAAAGC
CATTGTGGGCTTTGAAGATGGAAGAAAAGGCCGTGAGCCAAGGCAGGCAGCTGGAGAGAAGCTGGAAAGACAAGGAGTGGATTCTCCGCTGCAG
CCTCCAGGAGGAGCCGGCTCTGACGACACCTTCATTTTAGCCCAGTGACACCGTGTTGCACTTCTGACCCGAAGAACTATAAGATTATAAACTT
GCGGCCGGGCACGGTGGCTCTTGCCTGTAATCCCAGCACTTTGGGAGGCCGAGGCGGGTGGTTCATGAGGTCAGGAGATCGAGACCATCCTGGC
TAAAACGGTGAAACCCCGTCTCTACTAAAAAAATACAAAAAATTAGCCGGGCGTGGTGGCGGGCGCCTCTAGTCCCAGCTACTCAGGAGGCTGA
GGCAGAAGAATGGCGTGAACCCGGGAGGTGGAGCTTGCAGTGAGCCGAGATTGGGCCACTGCACTTCAGCCTGGGCAACAGAGCGAGACTCTGT
CTCAAAAAGAAAAAAGAAAAAAGAAAAAAAAAGATTGTAAACTTGTGCTGTTTTAAGCCATTAAGTTTGTGATAATTTGTGACAGCCACCATAG
GAAGTGCATTGAGATTTGTAGTATTTTGCGGGGTGAAGAGGAGGGTGAAAAGGAATGGACAAGCCTGTCAGGTCCCTGGATGGAGATTTAATGA
CGATGCCAAGACTATGGATCCTAGTGAGAATCCTGCATCCCCCATGCCCTACAGCTACCAGGGAGATGAGGCACTGGGAGAGTTAGTACTCGGC
TGGTGGTTTTGTGCCCTCTGGGCCTAGAAAGTATGTAATGCTGCCATCCCTGTATGCGCAGAAAGATGGTTAATGCAGCCCCACTCAGGGCAGT
TTGGGGGCTTACCTGAATTTCCCCACTGGGAGCCTCTCTCCTGACTTAACCTCAACAGAGGCATCTTCTTCCAAGTGCTGCTTGTAACTCAGCC
CCCAATGCCTGAAGACACACCTTCAGCCTCTCTCCTGACGTTCCCCTGCCTTTCCAGGAGGCCCTGGAGGGAAGGACCCCACACTGGCTCTCTG
CTGAGTGGCCCAGCTCTGGCCTATGGGACACGTGTGCGTTTGCCTTGCCCAGCACACGCTGGGGCTGCCACCATTCCCAGAGCAGTGCAGGCTC
TTGGCGCCACCACCCAAGTGCCCTGTCAATTTCCAGGGCAGGAGTCAGAGCCCCATCTGCAGGTGACTCTGGCTTTCTCAAGACTGAGTTGACA
CCTGTGCTCTCAGCAAACATGTTTCTGAGCACTCTTGAACTCACTTGCTAAGCTTGGCTTGGAAAGTGTGAAGGGAGAGCTGGGGTCCGCAGCT
GGAAAGACTCTCACCTCCTGCCTCTCTCCTGAACCCATTTGATGTCTCTCCTCTGGCCAAGGGGTAAATCTGCACATGTAAGTCTCATACACTT
TGGCTCTTCATGGAATTGGTCCCTGTAGAAGCCGGGCCAGAAAGAATCTAGCACCCTCTTGCACTACAATTTATTGAGCCTTTACTTTGTGTCA
GGCACCGTTCTAAGCAGTTTATATCTAAGATTGGATGGATTTCCACCACTCCTATTCCCATTTCGAGAAAGAGTACCCTTTCCCAAGACAGGAA
AAACAGCCAGGGCCATAAAGGAGGGCCTTTTGTGACATCATCAGAAGTGGAGAAGAGGTACCCAGATCCCATTAGCAAAGTGCAGCCGAAGAGT
CATGCTAAGTTCACTGTGTCCCTTGCGGCATTGCCTTCCTCAGTGCTGGCTGCCATCATACAACCCCATAGCTTGTGTGGCTTAAACAGCAGAC
ATTTATTTTCTCAGTGTTCTGGAGGCTGGAAGTCTGAGATCAGGATGCCAACATGGTCAGGTTTAGGGAGGGCTCCCTTCCTGGCTTGCAGACA
GCTGCTTTCTCACTGTGTCCTCACATGGGCAGAGAGTGAGTGAGAGCAAGCTCTCTGGTGTCCCTAGTCCCTCATCGAAACTTCATTACCCCAC
GAAGGTCCCATCTCCAAATACCATCACGTTGGGGGTTAGGGCTCAACATATGTATTTGGGGGGACATCATCTGGTCCATAGCAGGCACTGTCCG
GGTCAGAAGGCCTCCCAGGGATTTGGTCCTGAGGCTGGCTGTGGAAGGTGCCCTTGCTTCATCACCTCTGCACCTGCTCCACTGTAAGAACTGG
GGTCAGAAGGCCTCCCAGGGATTTGGTCCTGAGGCTGGCTGTGGAAGGTGCCCTTGCTTCATCACCTCTGCACCTGCTCCACTGTAAGAACTGG
GCAGACAGATTGGCAGCTTAGGCCAAGCAAGATAGATGGTACGGAGGCTCGACAGGAAGGTTTCTGGAGATCAGGAAGATAGGAATTAAGGAAG
GTGAAGAGGAAGAAAAAAGGACTCCATGAAGAGGAGAGGGTCTCTAAAAGCTGGAAACATAAGTGAGGAGGTGAGAACCAGTCTGAAGCACCCA
CCTGTGACCTGGTGGGGGCAGGAAGGTGTTAGAGGAGCTAGGGAGCTGACCAGACGGGAGGAACCCTTCCCATTCTTCATGGAGGCAGATTATC
GCTGGTCTGCAACTGGCATTCTTAGAGTTAGGAATCACTGTGTCCATTTTTCTAGACTTCTCTCATTGGTGATGATTTGACCTTTTCCTCTTGC
ACTAATGAGCAAGGTGGAGGAAAGAGATCTCTCTCTCTCACACATATACTATCTCACTCAATGCCCACCAATCCTAAGAGGCAGGAACAATTGG
GTTACGCCATAGTGAACTGCCCTTCTGTAGGTCAAAATTTAAATGTAGGTGATGTCACAGAGTTTAACCTAGTATTACCATCAATGCCATTATA
CAGATTAGGAGACTGAGGCTTCGGAAGGTTAAGTCATTTGCTCAAGCTCACACATTTGGTGAGTCAGGGAGCTTGGATGTGAACTGGGGCCTAA
CTCCCAAACCTGCTGTGGTAGGCTGCCATCCATGGAAACAGGGGTTCCTGCGGGAATGTGTAAGAGATGAGGTTGGTAAGTATGACTTTTAGAT
GCCAGGAGTTGACCTGTGTGGCTACCAATGGGAAGCCCCTGAAGACAGAAAGGTAGGGTAGCAAAGTAGAATCAACAGGGAAGGGATGAGGAGG
CCGGAGGGTAAGTAGGCCACTGCGAGATCAGGACGAAATTTACTGGGATCGTGAGATGCTGGAAGGAAGGATCAGAGAATCTGGGACCATGCTC
TGCATCTTTTTGCTGCAATAAATGGTATTGCATGAGATACCATTTATTGCCATGTGTCCTCATTGTAGTCATTTTTGTAATTTATATGACAAGA
AAAAATGACAGGGTGATTTTTACATATGTGCATACCTATTTACACACCTTCCAGATCAGGAGTGGTGATCTGAGCTCACTAGATATGCTCAATT
AGGTAATAATCAATTCAGGTCATCAAATTGATCACCCAAATCAATTTAGGAAATTGATGACCTCAATTAAGGCAAATATATACAATGAAATATA
TGCAGCTGTTAGAAAGGGCTAGATCCCATATACTAAAATGGAAAGAAAGCCCTTTGAAACCTAAGAAAACTGTGTTTCCATTATGTGAGCAGAA
ATCTTGTGTACACAGAAAAATGGTCTTTCTTCTTCCCCTCCCCTCCCCTCCCGTCCTCTGAGACGGAGTCTTGCTCTGTTGCCCAGGCTGGAGT
GCGGTGGCGTGATCTCGGCTCACTGCAACTTCCGCCTCCTGGGTTCAAACTATTTTCCTGCCTCCTGAGTAGCTGGGACTACAGGCGTGCGCCA
CCATGCCCAGCTAAGTTCTTTGTATTTTTAGTAGAGACAGGGGTTTCACCATGTTGGCCAGGCTGGTCTTGGACTCCTGACCTCAGGTGATCTG
CCCACCTCGGCCTCCCAAAATGCTGGGATTACAGGCATGAGCCACCGTGCCCAGCCAGAAAAATGGTCTTTCAATTGGAAATGTAATTCCAGAG
AATAAAGAATAGAACCACACTGTAATATGACAAGTAATAACACACACAAGCGCTAAAGGATTTTTTTTTTTTTTTTTTTTTTGCCTCCCGATAT
CTGAGGGGCAGGCTCCGTGGAGCCTCCAGGCTGAGAGGCTGAGACTGGCAGAGCAGGCTGGGTTCTGGATTCTGATACTCCTGGGCAGGAGTGT
CTCAAAGATTGAGACATTATGTTCATTCTTTGGGGCTCCTGTTCTTGGCACTATATCTAGCACATAGTAGTTGCCTCATAAATACCCATTAAAT
AAGAGCAGAACTAAACACATCTGGTTAGAAATTGTTCCTAGGCAGCAAGGAACCAACACGTGGAAAATATGGGGTTTCTGGAGCAGTGTTAATG
CAAGCCAGTTCCTGTAATGTACATAGTGACACCTACTGCCAATGACACGTGCTGGAGCCCTTCCACTGTTGATTTCCTATATTCACTTACCCTT
GTCTTTAAGTGGATGTTGGTGGAAACTTGAGGATACATCTTTGTGCAGCAGACAGCATTTGTAAGTAAATCTGTGTAGGCTTAGGGCCACATTG
ATAAAAATATATGAGAGTAAACATATACAGAAGCTGTTTTAAGAACTTCTGATGTCCTAGATGCTCTGATTTCTGGAAGCTCCATCCCACATCG
TAGCAAATATATTAAAACACTCTCATATTCTCTAAATACTCTTAAATAATGTGAGTGTTAAAACAATAGTTTCATTTGAAATTATTTGATGTTG
CGGAACTCACTTTCTAATGACTAGACTTCCCCGGCAATCACGGCATTAGGGAATTCATACCAAGTGATGTCTAAGTTATAGTTTCAAAATATAA
TGAAACATTTATCCATCAATACATTACAATTATGAAATTAACATGTTTTGGGGACCTTCATTTAAACATTTATTAATCTCAGATTTGCAGTATA
CTTTTTGTATTGTGCAGTCAATAAAATTTTCAGTCTCAAACATTTTCATAGGTCCCTGAAAAGCTCAACGCAAAAAACAGCCCCCATGTGTGAT
TCTCTACTGGGCAATAAAAGCCAATCGCGACCCTCATTTTCCCCCATTCCAGGGCTTCCCCCAGGATAGAACGCGGAAACGCTTCTTTTTCCGT
TTCCAGGGATTTCTTTCTTTTTCCAGTGTCGTTCTCCCTCTCCCCCACTTCATTTTTACAATAAATGTCAGTGCACACCCAGGAGCGGGTGGTC
CCACAGGGGACATCTTAACGCCCACCCAGGAGCGGCTGGTCCCACAGAGGACCTCTTAACGCTGACGCTTTCTGGAGAAAAGGATTTGTGATGA
AAATTTCCCCAAGGGGACCTTCTAGCGCCTAGCACCTTCGATGCTCCTGTTTCAGTCGCGGTCTCAGCGGGCAGTGGCCTCCTTTGAAGCTGGG
ATTCTCCTGCCCCTCCCGGGCAGCGCAGGCCACCGGTGACCCGGGACGGTCTGTAAACGACCTTCGCGTCCCCGTCCCCGTGGAATAACGCGCG
GCGGGCATCACTGCCCAATATCCAGCGCTCGTGAAGGCGGAGACCGGCGGGGCAGCTAGTTTCTGGTTGTGGATTCTGATTAATGGGCCGCAGG
CGCACGGTCTGCACCGTCCGGCTCGTTGCTAGTGGCTCGCTCGGGAGCCGCCGCACTCCCGGGGCTGTCCGCCCGAGCCGGCCTGGAAGGCAGA
GCACGCCTCCAGGTCGCTCCGCGGGGCCTCCGGAATTCCTGGGAAAGCCCCCCTGCAGCGCCGCCCCTGGACCCCAGGTGTGAGGAGCGGGGCT
CGGCCAGCGGCTCCAGAAGAGGCCCGGACCCCACTGCCGTGGCTGCCGGGGGCGGGAAAGTGATTTCTCGGAAAGCAGAGCACTTCGAAGAAGG
CGGGCCGCGCGAGCCAAGCTGACGCTATTGGTCGGTGTGGCCGTCGCTCTGCGCACCGCCCGTCCCCCCCACGACCCTTTTTAAGGCCGCGCGA
CCGCTGGCGCATCGCCTGGAGCGCGGCAGCAAGCGTGGGAACGCGGGCGGCGAGACGGCGGCAGGACGGCGGCAGGTAGGCACAGTGGGCGGGT
AGGGCGCCCGTGTCCCGCGCGGTCCGGTCCCGCGGGGTCCCCGACGCCAGGCGGGGCGTGGGGGTGGAACTACCTACCGTAATGTCCCCGGGCG
CAGCTCAGTGGCCACCCGTGTCCCTCTCTGCTTTTTGCCTATTTTCCAGCCACCTAAGTCCAATCTGAATGCCCAAGTCGTTGATTGTCGTTTG
CCTGTTTCCAAAGATTGGTAGATAGATGCCTTTTTAAAAATCTCATTTTTCTTTAAATCTGGTTTACATGGAAAACGTTAGGAGAGCTCATATA
ATGAACGGCAATAGCAACCCCCTATCTTGAAACGCGCTCTATCATCCCACTGAAATTCTACCACGTGGAATAATGCTTGGAGGGTCAGAGTTGT
GGAACTGCCCAATAACCAGTCGTTACTGAGGGTTAGTTTGTGAAGGAGGGGACAGACTGCTTCTAAAATTCTGTTTAATGACAGTCAATTAAGA
TTTCTGAGTCTGGCTTGAGGGCCTTTGCTTCCATCACAGCCCAGTCGTCCTTGGCAAGAGAGTCTGTATATGGGCCACAGCTCACAAAAGCATT
GTTTGAAAAAATTTATTGAAAGAACATTGTTTGTAAAATGAGTCCCAATACATAGGACAGACTTTCCTAAGGTGAGATGTGTTACTTACCCAGA
GCTGTGAAAGGCTTTACGGATGGAAACTAGAGACTGAATTTTCCAGAATTTTAAGAAGTCTCCCCAACCAATGGCCCCCCACTTTCTTTTTTTA
AACCAAAGGTAAGAACTGGTAGTTAAAATGACAGTTCAAAGCTTGTCCTTTTGGGTTACGGCAGCTTCCTCTATGAAAAGATTTGGGAGTGATT
CAGTCACTTGAGGAAGGTTGGTGCCAGAGAGAGAAATGGCTTTGGTGCTTTCTGGTCTAGTGATGCGGGTATGCTAGGCCAGAGCGAGTGCCCT
GGGCCCTCCGAGGCGGGGGAGAGGGTGGGGTCTGCTGCTGCCAGCTCAGGTGTGACCTGTAATCTGGATTAGGTGGGCCCAGGGCAACCTCTTA
TCACGTCCCTCCTGGAAAGCCTGACTGCTGTATACATTTCTAGGGCTGCAGATAGGGCTTGGCCAATAGATTACCTCAAAATCAAGAAGAGGAA
GTTATAAACATGTGATTAGACTTATTAATTTTTCCTGGCTGGGCATTTGGTGGAATTCGTTTGTTAATGTTTAATAAATCATTATGGATCATAA
CTGTAGTAGAATCTCAGGCTTTTGATGTTACTTAGAATCTTGGAGGAGACCACGAAGGACACACAGACGTGGCTGCACATGTGCAGAGATCTTG
TAATGTAACTATGGCTTTTCTCTGCACCATGTGTGAAATAAGCTGCCGCTGGCGTTGGGGCGCCGGCCTGGGTTCCAACACAGTCCGCTGGCTG
CCTCGGTATTTGCCGTGCCTCTCTGGGCCTCAGTTTTTCCTCTCTTAAATAAAGAGGGGACTGGATTAGATGATTTCTAAAGTATTTTCCAGCT
TTCAGATGCTATGACTTACTCTTCTCTTTTTCCTTTTAAAATTTCTTTTTCCTGTAAATCACCAAGATAGTTGTCCTGAACAGTTTAGATGTTA
TGAATCCATTAGGAATCCTGATAAGAAAGTGTTGACCCTCTTTCTAGAAAAATGCGTGTACTAACAAAATTTTTGCTTTTGGTTGCAGGAGGCT
CAGGAACTGTGGATTCTAGTTTAAGAAACTTGTAGCAAGGAAATCAGGGGAGTTGAAAGTCTCAGAAGCCAACCCCTATGTTGTGTTCTGGCCA
CCTCTGTGCAGCTCATTCCTCAATCTAGGAGAAATTTTTCTACCTTGCCCCAGGAAAGAGCTGCTAACTTGTTTTCGGGGAGTGTGGGGGGGTC
TTTGGTATTGGTGTTCCATTTCTCTTCTCAGGTACCCTCTCCCAAGTCTGTCCCTTCTTTATGATAAACACAGTGGCCTGCTTTAGAATTCTGG
GTCTCTTGAGATACAGCCTCTCAACTTACAGGTGAGGAAGCCTCAGGGAGAGGTTCAAGTGACGTCACAAGGTCTGGTTAGAGAAAAGCCTTTG
CCAGAGTTCAGAGTTTTCAAACCAAGGTCTAATTTGGGTGGGCGCAGTGGCTCACACCTGTAATCACAGCACTTTAGGAGGCTGATGTGGGAGG
ATCACTTGAGGCCGAGAGTTCAAGACCAGACTGGGCAACATAGCGAGATCCTGTCTCTAAAATGTATATCTGGGTGTGGTGGCATATGTCTGTA
GTTCCAGGTACTCAAGGGGCTGAGGCAGGAAGATCGCTTAAGCCCAGGAGTTCCAGGCTGCCGCGAGCTATGATGGCACCACTGCACTCCTGCC
TGGGTGACAGAGCAAGACCCTGTCTCTAAAGAAAAAAAAATTGAACTCATCCCCTTTTAAATCCATAAACGCACAACTGAGGTTGTTGGATGGG
GAATTTAAGAAATCCCACCTCCTCCTCCCTGGGACCTGGCCCTGTTTCCCGTCTCATGGCCACCTGCAGGGTGGCTGAAGTTTCCAGAATGCGT
ACTTCTTGTGAAGGTTCTGATTGATATTTTGCTTTCTGGACCAGTGTGGCTCTACCATATTTTGGGATGTTATGTCTTGTTACTGCTGATTGTT
TCAAACTCGTTTGCCCAAAGAGGTCACATTTTAATCAGAAGAACAAAAATGCTATTTTTTTTTTTTTTTGGGTGGGCGTCTATCCAGCTGTCTT
GGGGATTTCCAGCACTGTTCTTGGAAATGTTGGCTCTAAGTCGATAGGTCATGCTAGTTCTCTCTCTCTTTCTCTGTGCTGTTGGGGAAGCTGT
CTGCTTGGGTGAGAGTGTTCTTCCAAAGGATGTCTTTGGGTGCCCTGGGATGGTTAGGTCCCTAGAGTGGGACATGTTCTGGAAGAGGAGCCCA
GTGAACTTTGCCCTGTTCGGTAGCTGCAGTTTATTTCCTCACTCTTGTCCCTATCTTTCAGTTGGTCGTAGGAGTTGCCCAGGAAGGAAGTTTG
GATAATAATAATGACATCTGGCATGTAAGGTCCAGGCCCTGTCTCAGTTGCTTTCCGTGTAGCATCTGAATTCTCTTACAACCAGTACAGGTAG
TGTTTTGTTAGCTGCATCTGGGACTGGGTAGCTGATGCCCTGAGTTTAAATGCTGATCCAGCGTCTCCCAGCTGAGCGGTGGCGGGACTTGAAT
GAGATCGCGGTTCTGTCTCATTCTCAAACTGCTGCTCTTATCACTGTGAAGGAACAGCTATTTAGATGGGCTGGCCTGAGGGCCTCCTCTTTCC
CAGACGGCACGGACGCAGTATTCTATCAGGGAATGTGGGGCCAGCTGGGCATCTGGTAGCCCAGTTGGTTCTTCGGATAATGGGCTGCTCAGTG
AAGGAACCTGGTGCTGTAAAAGTGATTCCCCCTCTCCTTCCAGTCTTGGGCTAAAACTGGAGCTGTTCACTGTGGTTGTATCTGCCTGAAAGGA
AAGGGCAGATGAAAGCTGGAAGGAGCACAGCCAGCACAGTGGGAGGGGCCTGCAGGGGGCTGCTGGTGGCTGCAGGTGGCTGCAGGTTTCCCAC
GGAGCTTTCAGTTTGCACTCAGGGCTGTGAGGTCATGGAGGCCAGCATTGCCTTCTCATGGCAGGTGTCCCGGAGTCCCTGAATCTGTGGGTTT
CCCCCAAGCCAGCACCTTTGCTGCAAACCTCTGAGTTTCCTGTTAGCAGTTTTTGGGTTGCTGTGATGAATGAGACAATATCCGTAATATCACA
GCGTGTATTTCTGTCTTTCCAAGGATGTGTGACCGGAATGGTGGTCGGCGGCTTCGACAGTGGCTGATCGAGCAGATTGACAGTAGCATGTATC
CAGGACTGATTTGGGAGAATGAGGAGAAGAGCATGTTCCGGATCCCTTGGAAACACGCTGGCAAGCAAGATTATAATCAGGAAGTGGATGCCTC
CATTTTTAAGGTAAAGAGCCCAGCTCCCTTCCCCACTGTGGGCACAGTGTCTCCTTTCCCTCATGGACTAGTGGAGATGTTGAGTGACATCTTT
CTCATTTACTTAAAAATTAATCCAGTTTTTTAAAAGCAAACATTGGTTCAGGAGTGCTGAATGTGTCTCAGACATGTACATGAGCTGATTTCAC
ATCTCTTGAATTACGGAGTCACTTTTGTGGTCGCTTGTATTCTGATGAGGAGATCGAAGGCTCTTTTCTTCTCTTTTCTGCTGCTTCCTTTTCC
TGATGAGCCAAGTGGGAGAAAATACTTCGAGACCTTCACGCTAAGACCTTTCCCCACGGGCTTCCATTAGAGGCGATGCATATACCAGGGAAGG
TTTCAGTCCTTGTTCTTTCAAAGCTGATACTTCTGCCTGGCATTGTAGGGCCAGCTCAGGAGGGTGAGATGCCATCTGTCATTCCTTTGGGGGC
TGGGCCGTGTGGGGGGTTGTGAACCTGCAGCAGATGTCTAAAAATAGTCTCACCTCCCTTGAGAGTAATCGGTTCCCCATGCGTGGAGAGACGT
GATGGAGGGTTTGTTCTGTGCCAGGCCTGGCCCACGGCTTGTTTTCTGCTCACATGATCCTGTGTGGTAGATGCTGTTCCCACCCTCATTCTAT
AGAGGCACTGAGAGGAGAAGTGACTTGTTGAGGTCTCACAGCTCTTGTATGACGGAGCCAGCATTCAGATGCAAGATGGCCCAACTGTGAACCC
TTGGTCCTAACTCCAGAATAGCTGATCAACAGAGATTTCTTGCCATTACACCTAGATTCAGACAGAGGAACCAGGAATTAGGGCAGCCCAGCTC
TGCAGACCAGCTGGGCTATCGAATCCCTTGTGGGTCATCAGAGTCCTGTGTGGGCATCTCTAAGGCTCCAGATGTCCAGGTTTCACCTCTGGGA
GATTCGGAGCCCAAGGTCAAGGTCAAGCTAGACTTCCACGTTTGCCAGTGTGGACACTACACGTAGTCTGAGTCCTAGCCCAGGTTATAAGCCA
TTGCCATGTGTGCTTATTCTCTTGCATTTGGGGGCCAGAGATGGGGAGGGGAGAGTGAGTGTGGGAGAGGGGTGAAAGGGGCTGAGTTGTTGGG
GGCCAGAATCAGACCTGAGACCACAAACAAGGTGTGGAGGGGGTCAAGGCTGTAGGAGCCCATGACAGTGCTGGACTCCCAGCCACAGATGACT
GTCCCCTGACACCAGAGAACAAAAATGACAAAAGATCACACCTTAACCCCAGAGTGGACCATAAATAGGTCAAAGGTAGCTTAAGATGCATTTT
GGTCCTGCACGTCTCTGCCTGACCGATGTTTGCAGTGGGGCTGTGCGTTTCAGTAATTCTGTTAAATCTGCAATTTGAGGTTGCTGATCTTCTT
GAGAATCTGATGCCCATTACTGACCACGCCAAGGAATATGGGGACAAGGGAACGCTTTGCACTCACTTTCGAGGGGTCCCTTGGTGCCCCCACA
CGTTCCAGATTCTTTCTGATCAAGGACCCCAGGTCAGGGAGCCTCAGCACCAAGAAGTTTTGTGATGCCACTGCCTGCCTCCTCACCCTTAGTG
CCATTGTGGATTTCTCTCTTGTTTGATTGCAGATCTTTTTTTTTTTTTTTTTTTTTTTTTTTTTGCCTTTGCATCATGGTGAACTTCTCTTGGG
AAGGACTGCAAATGTAAACAGTGCCAGAGTCCCATTCAGTCGGTTGGGAACTCTGCATTATAGATAACCCCAAAAGCCGGAATTTCTCTGCCTC
GCCACTGTTGATGTCTGGGTCTGGGCAATTCTTTGTTGTAAGGGTTCCCTATGCCTTGTGGGATGTTTAGTGGCATCTCTGACCTCTACCCACC
AGATGCCAGTAGCATCCCCCTCCCCTACTGTCACAATCAAAAATGTCTTGGACATTGCCAAATATCTCCTGGTGGGGCAGAGTCTTCCCCTCCC
ATTAAGAACCCTGATAAGAGCTGGCCACTGCCAGGATCAACAGACTTAACCAGAGGTGTTGGGTGCTGAAGGCACAGTTGCAGGTTGGACATCA
GTAAAACATCTTTAAGACATGGCAGAGGGTGCTGGACTAGGCTGTGTAGCCCTGACATGTGGCATGCTGACGGAGACGAAGCTTCTCCCTTCAC
CCCATTCCCTACTTCAACTTGTGCTCGGGGGGTAATGAAATAAAGCCCCAGATTTATACATCTTATGCAATGTTCTTCATGGCCGTTTCAACAG
TGTTGAGAAAAATGTGCAAGATTCCCAATGTTTCCGTTGGTTACGCAGTCGCCTGATGTGGCAATCTGAAATGAGTCTACCATCTCAGCAGGAA
TGGGGCCAGGAGGGAGACGCGGGTGCCCACTGGAGCAGGGAGGCCATCGGGAGGGCCACCAGTGCCGGTGGAGACATGGCGCCAGCAAGAGCCC
ATCATGTGCTTCCCTGAGCTATGCTTCTTCCCATCCATCCTCTGAGAGGCCTGTCTCTGTGACGTTGGCCGGATAGATCTTTGGCTGTTGGATC
AAGTCAACCTCGGTCAGGTAGAGCCATCTTAGGGATTTTCCGGTTATGTAGACACTTGTTTCTTTCAACTGTTCAAAGAAAACCTTTCAACAAT
TTAGGAGTTAAGAAAGAAATTTATGAGTTTGGTTTCTTCCGTAATAAGTGGATGGTAAAAGGGGCCACTTATACCAGAAAATCCCTCATTGTCT
GCAGGCATCATCCTGAGGCCGGCCTCATGGTATAGATCACAGAAGAGAAATTGAAAAAACATTGAGCTCATTTTGTACCGACCCTGAGTACACT
GATGAGGCCCTGATAGCAACTCATTTAATTTTTAAAAAGTACTTTACCTTCTTGACATGACGACAAGGGGGGAAAATCTAGTTAAGTGAGGATT
ATGATTCATGGTTATTCCAGGCATCGTAACAGTCCTGAATGAGTTAAAGCTCACTTAGTAGCACAGATATTTCTCATTCAGCAGGCCTTGAAGG
GAAAAATAGCATTTAATTAAAAAAATGCAACATGAAACAATCATATTAAATTAGGAAGACCTCCTGGTCTTAGTTCAGTGCTGTCAGCCTGAGT
TGTGCGTCAACAGGAGAAATAAGTTATGGTGGAGGCTGAATGAGCCATAGTGACTGTTGCTGGCGGCAGGGGTGGGGGGGCCCTGAAATCACTC
TCATGTCTAGTTTTTGCTCTAGCTCAAGAAGGATCTCGAGAGATCATCAAGCTAAGTTGACCTTTTAATGCTGGCCAGAGAGGCTGGGAGAGGG
CCAGGTGCCTGCTGTGTCACAGAGTTCAGGGCAGGCGGCAAGCCAGGTACCCGATGGTGGCCCTGGATGCTTCCTGGCCGTGGGCCCTGCTGTG
ATGGATGATGTGGCCTGGACTTGCTCGTGGGGTGAGTGTGAGCTTGGCGTGAACGAGTGAGTATGGCAGTGCCTTGCAGGCACCGCGGTTGCGT
ATGTGTACCAGCGGATCACAACGTTGCCATGAAATCTGTGCATTTCAAGGCCCGTTTGGGATGTGGGTTAACTCAGTGGTTCTCAGGTGGGGGA
GGTATCATGATTTACCCTCCAGGGAATACTTGGCCATGTTTGGAGACCTTTTTTGGTTGTCACAGCTAGGGGAGGGGTGCTATTGGCATCTGGT
GGGTCAAGGCTAGGGATGCCGCTGAATATTCTACAATCCACAGGACCACCCCGCCACAAAGAAGGGTCCCAGCTGATATGTCAGGCTTGCCACG
GTGGGGAAACCCTGATTTAAAGTAATAAAAATTTGAAACTACAGTTGATTTAGTACATGGTTTGCTGGTTTATTTCTTTAAAAATAAACAACAG
CAACAAAACCCAAATTAATGCCCGGTCAGTGATGCAGTCAATGAAGAGAGCATTAAAGACCGTGTGCTTTAAGAAATAGTTTTAGAGGGGAGTT
TTACATCAATGTCACATGAGGAAACTACCACTTATGCTCACTAATGGGAATGTTCTTCCAGTCTCTTTACCCCAGGTAGAGGTGCAACATTTTG
CATCAACAAAGTAGGCAGCATTGGCCTTGAACAGTAAGCATTCTTGTCTTTTTTGAGAGGAAAAACAGTCTGGGCTATTTTAAAGGAGCTGTGA
CTCATGGGGGCATGATGGAGTTTCTGGAAGTGTGGTCTCCTGGGTAGCTAAATCCAAATCCCTGGAATACTGGCTAAAGACAGCTACCTGGGCC
GGGCGCGGTGGCTCACGCCTGTAAGCCCAGCACTTTGGGAGGCCGAGGCAGGCGGATCACGAGGTCAAGAGATCAAGACCATCCTGGCCAACAT
GGTGAAACCTCGTCTGTACTAAAAATACAAAAATTAGCTGGGTATGGTGGTGCACACCTGTAGTCCCAGCTACTTGGGAGGCTGAGGTAGGAGG
ATTCGCTTGAACCTGGTAGGTGGAGGTTGCAGTGAGCCAGATTGCGCCACTGCATTCCAGACTGGTGACAGAGTGAGACTCCGTCTCAAAAAAA
TAAAAAAACTAAAATAAATAAAAATAAAAAATAAAAAAAGACAGCTACCGAGACCCAACCTCCCTTCCCCAGTGAGAATCTTGAGGGGCAAGGC
CCAGGAATCTGCATTTTCACAAGCTTCCCAGGGGCAGGGGGTTGGTATATGCATTAAGCCTGAGAACACTTGCTGTGGTGGGGAAACTGTTGGG
TTGGAAGTGGGTACAGCTGGCATTTAATCCCAGACTGGGTGGCCTTGGGAAAGTTACTTACCTCTTGGAATTGCATTTTCCTGAAGTGGGTACA
GCTGGCATTTAATCCCAGACTGGGTGGCCTTGGGAAAGTTACTTACCTCTTGGAATTGCATTTTCCTGAAGTGGGTACAGCTGGCATTTAATCC
CAGACTGGGTGACCTTGCGAAAGTTACTTACCTCTTGGAATTGCATTTTCCTGAATGGGATCGGGATGTCCCTACAGATCTCCTATCATTTTAG
GCTGGAGGACACTGTCTGATTTTTCCTGAAACCGAAGATAATTCTCATCTGACTGTGGTTACCTCCTAGTTGGTGTGTCTGTATTCCAATTTTG
CAAGCTTAAAATCTGTCCATTCACCTGTCTTATTTTTTTAAAATCTGTACTGCCCCATTCTACATTATGACTTTAAGATTTCAGCTTTGAATTT
CTGGTCCTAGTTCAGATCCTAAGGTTTGGAAAAGTCAGAAAACAGATCTATTAAAAAAAACACACACACATTAAAGGGAAAGTACTTCATTACA
TTTTAAACAGCACCTGGGGGGAAAAGTCTAGAATAAATGCAAAAATGCAAAACAAAATTTCTATTAAAAGTAACAGGGCGATGGATGACCATTT
TGACTTTTTGCAAATGTTCCCTTTTCATGCTAAATTTTAGGGGTTGATCTGAAAAAGCAACAACTTGTTCTTTGTTTTGAACGCTCTGGTGAAA
CCATATGCAACTTCCTCTTTGTGCTGTGTAAACCTCAGGTGTCTCCAGCTCTTCAGCCAGGCAAGGGCTTGGCTAAAAGGTGCACTCCAGGGTT
GGAGGAGCCTTCAGCCTGGGTTCGACTTCTGACTCAGCTGTATGAACCTGGGGGACCCCCTCCCTGGCTTGGGTCTTTCCACATCTGGTCCCCA
GATGTGAAAAGACTCAAGACATCTGATTGGAGTCTCTTTGGGTCCTGAAATTGAATGAGAGTTGATGGCCAACAAAGATTCATGGGAAGGGAAG
GATGTGTGGGGTCCTGGTCATGGTGACGGATATTTGTGATTGGAGTCGGCCATGAATTTAATGTGCTTCTGTTTCTTTCTTCAGGCCTGGGCAG
TTTTTAAAGGGAAGTTTAAAGAAGGGGACAAAGCTGAACCAGCCACTTGGAAGACGAGGTTACGCTGTGCTTTGAATAAGAGCCCAGATTTTGA
GGAAGTGACGGACCGGTCCCAACTGGACATTTCCGAGCCATACAAAGTTTACCGAATTGTTCCTGAGGAAGAGCAAAAATGTAACTATCCTTTA
TGGGCATGAAACCTTCCAGAAGCTGCCCTGGCCTGTAGCCTTCACCACAGAACTTGGGTCTGGGGTAGACACAGGGTCTGGGGCACATAGTTTC
AGTTAAACAAAGCAGTTCTCTCCTTCGTGTAAGCAGAAATTGAGATCTCTTTCCAGTGACCTCAGAGGGAAGGGCGGAGGAGATTGGGTGCTGC
CCAACCTGTGCCCCACTTGCTGCCTGACCACAGACATTTGTAGGGCCTCCCTGGACAGGAACAAGCATCCTCCAGCAGTTTCTGATGAGGAATC
CTTTCATTTTCCAGCAGCTTGCAGAGGAGGCCAGGGGAAAATATGGTGACCACAGAAGGCCTGGATCATGAGGGTTGGGAGTAAAGCTCTCAGA
TGAAGTTGTCAAAGATGGCACCATGGCCTTCGTTGTTGGAGATGGGATATGCCTCAGTGTGTTACTAGATCCTGATTGTTATTTGGGAACTCCG
GGATGTGATCATTCCTGAGGTTTCTTCCAGCTCTCAAATCCCAAGAAAGCATTTGTGACAGATTAAAGCAGGACAGCAAAAACATGCCGCACCC
TGGTCCTACATCACTAAAATAAATCCCAAAGTACTGAGCTGGGATCTGGCCTCAGAAGGCTTCTCAAAGCCCTGTGCTGTGCCGCTGCCATGAA
TTGTAGCTGATTTATGAGATAAAACCTAATTACCAGCGCTGGTTTAGAACATGCTTTTAACACATTGATTTTCTTAGCCACTTGATGAGGCTTT
TAGAATAACCCACTCTGGCTTTCTCCTTCTAAGCTTCTGAGTAGTCCACGAATGCCTGTAAGAAACAGAAACCCCCAAAACTTCTTGGGGCTGT
GCAGAAGTTTGAAGTTTGCACTCTTCCCGCTGTGGGGGTGTTGACTTAGAATTTTTTAAGAAGTTAAAATGGTGATTCAAACAAATCTATAGTG
AATCTGTGTCAGAGAGTTGTGTAATCGTCATGAAGGAGGCAGCATGATGGTAAAGATGGTGGACAGAGAATCCGAGGTGCTGGTGAATGTGGCC
CATGAACGAGAAAGCTGAGCCAAAGCCGATGCCCGACCCGGACAGTACAATTGCGGTTTGGGGCGTTTGTAGCCTAATCGTCTGCAATTAGCAC
CTAACTCTACTGAGCGTGAACCCTAGTCAATACTAAATAGTAAGGCCAACAAACTGACCTTCCCTTGGAGAATCGGGTGCCCTTTAGGAGGAAA
CCTCTATATAATAGTCTGGGGTGACCCTGAAAGGCTAAGCAGTCATCTTTTTGCCTCATTTCCCAGTTTTGGACAATTTTTTTTATCACCAAAA
AGTAAAGTGTGATTTGAAGGAATAGAGAAGTTTCTTTTGGCCCCTACTGCCACCCCCAAGATCAGTCAGGCTTGAACCGGCAGGTGACCCCAAC
TTGTTTATAGCATGGGGTGCAGCCAGCATCACCTGCTAGTGATGTTAAAGAAGCAGCAGGAGGTTCTCAGGGGTGAAGCAATGTAGCATTTGCA
TAATTGCCTACAGGGGCCCACTCGCCTCTAACCTCAGTGGTTTGCAGGCTCCATGGTGGGCGCCCCACCTGCTCCCTACTTCCCCATCAGTAGC
TTCTCAGTGAATTCTTTAATCTATCGAAATGGAATCAAAATTCAGCCGGGAGGAAACAGTGGGTAAATTCTGTAGGTCACTGGGAATAATGAAT
TTTTGTAAGTGTTGGTTGTGATACGTCTCACACTGATAAACTTCATAAGGTAAGTTGTCGACTGAGGAAGCGGCATGAGAAGGGAAAGAGCAGA
GGCTGGCCCAGCGTGCCCCTGTGGACACTGTGCACCTTTAGGCGGTTTGTGATTACGGCTGGTGCCCACTGGGCATCAGTGAGGGTGCTGCGGT
GTGTGTGTGTGCATGGCTGTGTGCACTGATGCACATGTGTATATTTGGAAAGGCCAAAAAGAGATTTCCTGCCAACTGGCAATACAAAAAAGAC
CCCAACCTGCCAACACGTTTTGCCAGGAAATCGGTGAGTGTTTTCAAATGCCCTGGCTTATAAATGTAACCTTCTTAAGTTAGCAAATGCTTGC
TGTAAAGTATATCTGATACAGACTCATTTTGGATCTCAGAAAATAGAAGAAAGAGACCAAAGTGCTGTCTTTATCCTTTAAAAGTGTTAGCAGA
AACAACCACCACCACCACCACCACCAACTTCTCTCTTTTGACTTTGCTGATCAGATGACTGAAGCATGGGAAAAAAATTCTGTGCCTTTCCCAA
ACCAATGAAGACACTCACTAACTTAACGTTTCCTTAAACTCAGCATTTACAGAGTAGATTATGGCTTCAGCAAAGGCTGTGATGCCTCCGTGCC
ATGTGTCATGGTGTTTGTCTGGTTTTCTGTAGGCAAACTAGGCGTGGCAACTGCTGGCTGCGTGAATGAAGTTACAGAGATGGAGTGCGGTCGC
TCTGAAATCGACGAGCTGATCAAGGAGGTAAGCAGAGGCAGCATTTCAGGGGTCTGGCCCTGCCAGGAGGAGTCTCATGTCTTCTGGCAGGGAG
GGGCACCGTTCTTGCTGTATGAAGGCAGCCAGACCCTCGGGCTCGCTGAGGAAGTGGCATCTCCACCTGTACAGATCTGGAACGGAAGGACTGG
CTGGGCGCCCCCTCCCCAGGCCAGCTTCTGCCGACAGGTCAACCCCTCACGCCAGATCCCTCCTTCATCCCTAGGGCTGGGCCTGAGCTGCCGG
TTATGTGTCAGTGAAGTTCCTTCCAGCTCCAAACTGTTCGAACACTTTGGACAGCAGTCTTTTAAATGGCATCGTGAATTCCCTTGCCTTCCTA
GCAGGCTACAGAGAACAAAATGTAAACTTTCCTTGCTGCCTGCGCTTGTCATGATGGCGGGTGTTGTTGTACCAGCTCTCATGGCTATTTGATT
GCTTCCTTTTTAATGTGCAGTTTTTATTCCTTAACGGATTGGAAAGCTGGATAACCAAGCATGTTCACATTGCACATAAATTCCTAACAGGCTA
CAAGGGAGAGCATAATCGGGGCCTCCTTAGGCAAGAATACATGGCTTTTGTATACCCATTTCAGCTATTGGGATGCCCTTTTCTTCTGTCTTTG
GCCTCTTAACTATGCTTTTCATGGGTATTACAAGGTGGACATTTGCATGACTAAACCTCGTGCAGCCTTTGCAAAAGGTTGTGTGTGAGTACTG
CATAAATTCTTCTTCTTAATAACATAGCAATAATGGAATCCTTAAGAGTAGCTTGGCTGTCAGAGAATGAAGATAACAGAATGTAACCTACCCG
AACGGCCTTGCTCTTCTGCACAGGCAGCTGCCAAGTGTCTGGGTGGTCTTTGGCAGCCGACTGAGTGAATTCTTGGCTTCGTGGCCCTCCACCA
ATGTCCTGTTTTCTGTAGAAGGCTTTTCTGCTGACTCAGCTGAGAGAGTGAGATAATCGGATTGCCACACCGGGGTCACAAGAAAAGCAATTTC
AAAACATTTCATAATTGGATTTATCAGCCTCCTTTAAGGAATGGGCCAGAGACTGAACATCTGCATGTGGCTCGTTGAGCGAGATTCTTGAAAG
CATGGCAGCCTTTGGCCAGCATCACTGTTCACTGGTGTCTCAGAGCTTGTCTGCGTGTCCAGGGCGTTGAGGGCAATTGGATAACCTGGAACAC
TGGGTCTGCAACAGGCAGGTCCTGGAAAGGGCCAGGAAACTTATTAGTGGCCCCCAAAGAGCCTAGTAATTTGGCCACATGGTTTGAATGCTAA
GCCTGGTAGATTTAAGGCCTAGGGCCTTTCAGGTTGCAAAGTGAAACTTGACTTTTGTCTCCTGAGATAAAGGTGACAATATGGTTTTACTTAC
ACATGAACTGTTCATTTTTACAAACACTGGAGCCCCAGGTGGGAGATCAGTGACTGAGCTGCCCTCCTCTCCCTCCCCTGACTGTGCAGCCTTC
TGTGGACGATTACATGGGGATGATCAAAAGGAGCCCTTCCCCGCCGGAGGCCTGTCGGAGTCAGCTCCTTCCAGACTGGTGGGCGCAGCAGCCC
AGCACAGGTGAGGGTGGGTGGCCTAGAATTGTCACCAGGCATGGCCTGAAGGGTTTACGGCATTCCACCAGCCAGGGACGGAGTGGGGGTGGTT
GTTGCTATCATGATCCATGTCTCATTAAAGGTAGCTCAGCCCTGAGAGGTGAAGAACGATGGCCCTGGTTTCCCAACATGAGGGCAGAGCCCAG
CTGTGCCTTTGGCCCTGCTCTCCGCTGCTTCTCAGGAGGGCACAAGCTGCTTGTTCCCTAGGTGATGTCCATGCCAGCTCTCCCCACGCTCCTG
GGAAAGTCCAGGGAAGAGGGTCTAGTGAACTTGTGTAAGGCCAGAGGGAAGATGCAGCTCTTGGCCACGCTGGCGCCCCCTCCCTGGGTACTCC
CTTAGGTCGGCCAGTCTGTTACGACATTAAGGACCCCCTCAGAACCAAAGAGAGCCCAGGGCATTTGGAGGAGAGGAGAACTCAGACCAGGAAC
AGGAAGTTTGTTCAGAGCCCTAGATTTGAATCCCACGCTGGGAAGTTACAGATCTTGAGCCTCAGTTTATTCATCCGTAAAACAGAGAAACCAG
GACTTGCGCCATGGGGTCACGAGGGTTAAGTGAGGCATTCATCCTTTGATTCTCTTTTGATGAGCCACTCAGAACCCCCAGCTGCTTGAAATGC
CCCCGCCCCCCGCCAGGCCCAGGCTTCTGCAGCCCGTGAATGTGTTTGCTTCCTCCCTCGGCGTCTTCCCCTAGCCCTACATCCCCTGTGGGAG
TCTGGGAGGTGGCAGCTGGCCCCGGAGGCCTTGAAGGTTGCCAGGCCTTGGGTGTCGAGTAAGAGGTCCCTGGAAAGGCACCTCTGTCTGTGCT
GCAGGAACACCATCCGGAAAGCTAGCTTGCACCCCCAGGCTCTCCCCCAGAGATAAGCCCTGGGTGGTGGGTATGCAGTCCAGTGGTATTCTGG
GGCAAGTGCTTGAGGTGGGAAGGAAATGTGGCCCCACAGTGTCACATTGACACATGGTGCAGAAATCCCACAGCATAGAGGAAGTCCCCCCGAC
TTGCCACTCTGGTTTTCTGCATTTGTGAAACAATGGCTCTCTGCTCTGGTGGGGAAAAGCACTGAATTCAGCGGAAGCCTGTGCTCCCTGGAGC
CTCTGGCACGCCATGTGCAGCCTTTTAAGGGACTTTTGGAGAAGGAGCGATTGGGGGTTACTCCCTGTACACCACACCTGGGTGGCTCTGACTT
GCTTTTCTGTTTCTCCTGCAGGCGTGCCGCTGGTGACGGGGTQACACCACCTACGACGCGCACCATTCAGGTACGGGGTGGTCCGGCTGAGAGG
GGCGTGGGCACTGTTTGAAGACAAAGCCCAGATAACCCAAGCAGGGTTGCGGGGTTTCGAGGATGAGCAGGACCTGGTGTGGAAGTCTAGGCCT
GGTTCCAAGGATGAGCAGGACCTGGTGTGGAAGTCTAGGCTCCGTTCCGAGGATGAGCAGGACCTGGTGTGGAAGTCTAGGCCTGGTTCTGAGG
ATGAGCAGGACCTGGTGTGGAAGTCGAGGCTCCGTTCCGAGGATGAGCAGGACCTCGTGTGGAAGTCTAGGCCCGGTTCTGAGGATGAGCAGGA
CCTGGTGTGGAAGTCGAGGCCCCAATTTCTCCTCCTCACCATTTGGTCAGGAGCTGGCGAGTGGCCTCTTTTGAGGCCACTTTTCTTGGACTGG
ACCAGCAAGTAGACAGAGTACTCTTTGAGGCTGTAACTGTGCAGGAGAGACAGTGAGGGACAGGCCAGCGTCTGTCCCAGCGCTGCCTCTCTTT
TGGGGCTGCAGGTGGAGTGATGGAGCCTCTCAGAGCCTCATCTCAGGCTCCCCTGCCCACCACCTCTAGGTGGATGCAGCTGTTCCCATTTCGC
GGGCTCAGGTGGTCACGTACTTGTGTGCTCCAGACTGGGGGCCTCCCCGCGGTGGGCAGAGGTGGCCTTTGTGTGTAGTCTCAGCGGCTAAGGA
CCCTGGCTGCACTTCTGCCAGCTGCCCCTGGAAGTGACGCTCAGGCGTGGTGACGGCATCTTCTGAACAAGGAAGCTGGGGCATCTTCCAGCAT
TGGCTGGTTCTGTGGGAGGCGAGCAGCTGGTCTCATCAGATGGGCCACAGAACGTTAGGGCTGGGCCTCTGGGCAGGGGAGGAAATGGGGCAGG
GATAGAAGCGTGCTGGCTGAGGACTGCATGGGACAGTTTTGTGGGTAGCAGAGGTCGGTGCTCACCCAGGCAGGAAAACTGTCGAGATGGCAAT
GCCCTGTCCATGAAGCTCTTGTCTATTTCCTGGCAGCCCTTTGAGCTGGGGGGTTGTTTTCACTCCGGCTTCACAGATGAGGAGACTGAGGCTG
GGAGGTTCAGGATCTCATCCAGGTCGCGCAGGCCTCTGGCTGGAGCCTGTTCTTGAGGCCTCCTTGTGCCCGGCAGCCGCCATGCTCACAGGGT
CTCAGTTCTGGGAGGCCTGGGTCATGAGGCTAAGCCTCCGGGTCTGCCAGCCACTGGGCTATGGCCTGGGAGTCTGCTGACCAGTGCTTTCCGG
GGGCTGTGGAGTAGCCAGGCACAGAACTGAGCCATCTCGGTGAGATGCAGAGGCACAATTTGGGGAGAGCATGTCCTTGAATTAGTCCAGCAGG
GTCATAGCCACTGCCTCATGCACAGAACATCCTTCTAGAAGCTGTGAGCTTTGGTACCTCTGTCCAGCCCCGCCCCCACCTCTATGTAAATCCA
TGCTTCAGTTTCCTTACCTGGAAAATGGGTATAATAATAATCTACCTTACAGGGTTAAATGTGCTGGTAGGTCCCAAGCCCTGGGCACAGCACC
TGATATGAGCTGTGTAGACCATAGTCTGTGGTTTATAACTTCAGGAGTCTTGTCTGTGTAGATAAGCATTTGTCCTGTTACCCAGTTTCTTTAA
CTGGTGCTTACCTGTGCCGAGCACAAATACAAGCTCCGATAATCCTCACAGCAGCCCAGTGAGGTGGATGCTGTTTTTATCTCTACTTTGCTGA
TGAAAAGCTGAGGCACAGAGTGGGGAAGCGGCTTGCCCCAGGTCATCCAGCTGGGCAGCAGCTGGGCTGGGACCGCACGTCAGGCAGGTGGGCT
GCAGGGTCTGTGCTGCGGGTGTCTTTGTTGCTGCCTCTGTGGTCAGGCCGTTCCCCAGCAAGGCTGTTCTTGTGGGTGAGTGGCTCTATAAGGG
CAGGGCTGGGTCGCAGCAGGCCCTTACTGTTGCAGAAGGCTCACCTGCCTCACCTTGGCAGCCCTTGGTGAGCTGCTTCTCACCTGTCAGGTAC
AAGCCTGACAGCTGGAACCTTCTCTTAGCATAAGACGTTCTGGGGTGTGTGGTCACTAAGACACTCCTGCTTAGGCGTCTCTGAAGCACTTAGC
TGTTCCGGGTGAAGCTGCCAGTTCACATGACTGAGAGGTGCTGGTACCAAGATAAGGCCCCGGAAATGACAGGGCAAAGGGCCAAAGGGGTGAC
GGAGTTGCCTGAAGCGTTTCATTCATTTGGAACAAAGTTCGAAAGTTGCTTCAGGGTAAAGGGAGGGGAGTGTGTGGAGTCCGCAGGCAGTGCA
GAAGTTGCAGACTTGGCGTGGGGGGAAGATGATGAGTGAGGAAGAGCGAGACCAGCGGGGAAGATGGTGAGCAGGGAAGAGCGAGACCAGTGCT
GCCATTTCCTTCTGGGTGACCCTATCTAGGATTCAGTTTCCTGAAGTGTCAAATCAATGAGGTGGACCAGAGCTTCTGCAGCTCTAGCTTCCTA
CCCTGCATGATTCTAGAACCTGCGTAACTCCAGCATGGCTGGAAATGGAGCCCGTGAATGGTGGGTTGTCAAATAATAAGGCAGGGAGCAGAGC
CCAATGGGAGGGTCCAAGGAGTTCTGAGTGCCCAGCAGTGTCAGCAGGGAATGCAGATGGACTCAGGGCCTCCCCAGATGTGGTCAGCCTTGCA
CAACTCTAGGGGGCGCCGTGGAGTTAGGCACCTCACAACTTGTGTGGCTCCACAAGGCAGCCTCGAACCGGTCTGGGGTACTTAGAAGGAGCTG
CAAACTCACTAAGCTCATTGCTAGCGTATGTAATTCTTTTGGTTTAAGGCAATTAAAATGCAGATACCTCCCTGCATTTACATGTTATCTGCAT
TCCTACCTTAGATTAGTGTTTTCCAAACTTTGGTCACTTGAGGTCCAGTGTCACAATTGGTTTTCACAGCCTGGACTGTTACTAACATCATGTT
TTTCCTCAAATCTTAATCTTAAAATAAAACTTAAGTTTATTTTAAATGAGAACCCACTATCATTGGCTGTAAAGGAAGACCTGTGGCACCTGGT
ATAAGTAGGTAATACTGTCTGGCTAGAATTGATTAAGATTGTATTTGTCTTTCATTGAATTTGTCTTGACCTCTGACAATGCCCTGAATTTGGA
GCCCTAAGTTCAGTGCCATTATTCAAAAGGGAGGTTGACAGGTGTTAGGTGTTAGAGAGCTATCAGCACCAAACTGAGGCTTGCTTTTTGAGGT
AATCAGAGGTTGGAAGAATATTGAAGATGGAATAATCTTGTGATTACATGTATCACCCCTCCCCCATTATTCTGTGTGTGCCACTTTTGGGGAA
CCCCTGCTTTAGTTTGAGTGTTTTTCAGACTCTTTCCTGAGAACAGATTTGGATGAAGGGTTTTGCAGAGATTCTTAATCTGAAATCCAGGAAA
TACCAAGGGCGCTGTGAATTTGTTGAGAAAAAGATTATGTCTTTATTTTCTTTAACTTCAGGTTTAAATTGAGCATTTCCTTTTGTTATGAATG
TAGGCAGCAGACCGCAGTGCCCTTGGCAGTGCCTATGAGACGGAGTATTGTCATAAGAAATGAGTTGTCGAAACATCGTTTGCCCTCAGCTTGA
CGTGTAAATTACAACAGTTACCAGACCTTCTCTAGACCTTGTATTTAAAGAGTCAGTGAAGAAGTACAGAGAACACTGTTGTAGTTAATATGTT
CAGTCATTGGTTTCCTTAGTCATTCTGTGTATTTATTCATGCATATAAAAACATTACTTCTACAAGCAGTACATGGATTTCCCCAGAATATCAA
AGTGGTTCAAGACACACAAAGAGTTACCCGTGTAGGTGTGAGACAGACTGTGCACTTGGGCAGGAGGGACCCTGTATGTCTCCCCGCAGCACCG
TCATCGTGTCCCTCTTGTCCACAGCATTCTCCCAGATGGTGATCAGCTTCTACTATGGGGGCAAGCTGGTGGGCCAGGCCACCACCACCTGCCC
CGAGGGCTGCCGCCTGTCCCTGAGCCAGCCTGGGCTGCCCGGCACCAAGCTGTATGGGCCCGAGGGCCTGGAGCTGGTGCGCTTCCCGCCGGCC
GACGCCATCCCCAGCGAGCGACAGAGGCAGGTGACGCGGAAGCTGTTCGGGCACCTGGAGCGCGGGGTGCTGCTGCACAGCAGCCGGCAGGGCG
TGTTCGTCAAGCGGCTGTGCCAGGGCCGCGTGTTCTGCAGCGGCAACGCCGTGGTGTGCAAAGGCAGGCCCAACAAGCTGGAGCGTGATGAGGT
GGTCCAGGTCTTCGACACCAGCCAGTTCTTCCGAGGTCTGTACCGTCGTCACCTTGCTGCCCCCACATCTTAGAGATCACGCCTCCTATCTGTG
TGCCATTTGCAGCTCAGGGTGGCCCTGTGGTCTCATGGAGGGATGTGGAGGAGGAGTGAGGGGCATGGTGTGGCAAGGAGGTGCTCCTTTGAAT
GGCACTCTGCCCTCTCCACTGAGCGATCCTGGGGTGCTTCTCTGTGCCTCAACTTCCCCATGTGTACAGTGGGGGTGCAGAATCAGTACCCTCA
TCAGGTGATTGTCGGGAGGGTAAAGGAACTTAACCTCATGTACCTTGGAGTATGTTCCAAGGATGGTCCCTGAGCGCTGGCTAGAGACACAGAG
TCTGAGGCCCTGCTCCAGCCACAAGATCCGAACCTGCATCTTAGCAAGGTCCCTGGGGATCTGTGTACAGGTTTTGAGACACGCTGACTTTCCG
CACACTAAGTGCTCAGGATGGCAGCTGCACACACTAAGTGCTGCCTGTTAGCTTTCGTTACGCTGTAGGGAGGATGTGTAGGATACCAGCCTGG
GTGTCTCCTGGAGGGTTAGGATGGCGTCTCCCTTCTCCACGTCCCCATGCTCTCTGCTCCTCTCCCTGTAGCAGCACAGCCCGAGATAGTGTCA
GGTCTCAGCTGCCACTGGCCTGAGAACTGCCAGAGGAGGGGCCTGGAAGGGCGGGTGCAGGGGTATGTGTGAGCTCAGGGGCATAAGCCCTGAG
GAATCCCCCTAGTGAGGAGGTGTGAGGCCGCGGTTATCACCCCTGAGCAGGCAGAGCGGCACAGAAGTGCGTCCTGCAATGTACATTCCCGGGC
CTACCTCACATCCTCTGATTCCATGGGTCTGAGGCGGGGCCCAAGATTCTACATTTCTAATGAGCTCCCAGGGAAGGCGACTGCTGCCGGTCCA
CCGGCCCACTTTGAGACCCTTTCAGTTGGCTGTCAGAGATTCGGGTGAGGGCTGCAGCCCTCCCTGGGTGTCCTCAGGTCGGGGTTGCCAACTC
AGTTGCCTCCAAGGGCCAGGCAGGTAAGAGCAATGCACGAATTGAGAGATAAGAGAGAGCACTGGGCAGCTGTGCCCATGGAGAGGCGTTCAAA
CGAGGCATTAAAATCCCGGCAACAACCTGGAATGGCCAAACGAACAGCTCCTTGCTTGGAATTTGGCCCAAGGGCCACCAGTTTGAGATCCCAT
GGTGCCCTGGCCTAAATGCTACAAGCCCTGGGCCTCCTGATCCCCCAGCCCTGCTGCTGCGGGAGTTGGAGGTCATCTCGGCCTTGCTTGCAAA
CACCCTCTGCCTGTGTCATTCCAGAGCTGCAGCAGTTCTATAACAGCCAGGGCCGGCTTCCTGACGGCAGGGTGGTGCTGTGCTTTGGGGAAGA
GTTTCCGGATATGGCCCCCTTGCGCTCCAAACTCATTCTCGTGCAGGTAAGTATGGGCAGCTTTTTTTTTTTTTTTTTTTTTTTTGAGATGGAG
TCTTGCTCTGTTGCCCAGGCTGGTGTGCAATGGCGTGACCTTGGCTCACTGCAACCTCCGCCTCCCGGGTTCAAGTGATTCTCCTGCCTCAGCC
TCCCGAGTTGCTGGGACTACAGGCCCGTGCCACCACGCTCAGCTAATTTTTTGTATTTTTAGTAGAGATGGGGTTTCACCATGTTGGCCAGGAT
GGTCTTGATCTTGTGATCCTCCCAGCTTGGCCTCCCAAAGTACTGGGATTACAGGCGTGAGCCACCTCGCCCGGCAGTCTGGGCAGCTTTGAGC
CCTGCATCCAGGTCTCCTGGCCCTGTCATGTAAGGATCAAGTTTTGGGCTGGAGAAGAGGACACAAGGACAAGTTTCCACCTGTGTAGGAGGCA
GGCCTGGGCAAGACCTTCAATATGCGGGAAGCGGATTCTTGCCATCTCCGAGCCATCTTCTGGTGTCTGGGACGCGCACAGTCTGTGTGTGTGT
GTTTTCTTTTTTCTTTTTTGGTTTTTAAATCTAAGCCTCTTCTCCAGAAAGATTCTACCTGTTCAGTTGCCCGAAGGTCTAAGTGACAGATTAG
GGCATTGCCAGCCCTTATTTCATGTAGTCAAGGGAATGAATCTCTAATGGTGGGATCCTGGGTGCTGGCTGTCCCAGCTGGGACAGTACTGGGG
AGTATGGAACAGTTTTGGCAGGAGTACTGCTTGCCCTTCCTTGGCATTCTGGCTCATTCACTTGGGACTCACGTGGCACTGGGGTCATCAGAGG
ACCTGGCTAGGGTCAAAGACAGTGCCCACCCCCTTTGGAGCCTCCGCCTCTGCCTCTGACTTTCTGCACCTCCCATCTAGATTGAGCAGCTGTA
TGTCCGGCAACTGGCAGAAGAGGCTGGGAAGAGCTGTGGAGCCGGCTCTGTGATGCAGGCCCCCGAGGAGCCGCCGCCAGACCAGGTCTTCCGG
ATGTTTCCAGATATTTGTGCCTCACACCAGAGATCATTTTTCAGAGAAAACCAACAGATCACCGTCTAAGTGCGTCGCTTGGGCGCCCCACCCC
GTCTGCGTCCTGCATCCATCTCCCTGTTACAGTGGCCCGCATCATGATTAAAGAATGTGGATCCCTCTGTCTGGGGTGGGATGCCTTACTTTGC
ACTTAATTTAATAAGGGCATTCTCGGAGGAGTAGACGTTTAATACGAAGTGGCGGCATAGCCCTGCCGAGATGTCGGTGATGGCCTGGATGCTG
TAACCACAACCTGTGGCTAAAAATTTTATTTTCTATCCTTTACCCGTCATTATCATTAGTTGCTATGATTCTTTCTGCATTTTCGGTTAACTAT
CATTTCCAAAGACTTGTCATTCAGTAATATTAGCAGATAGCTGCTTCGATAAAGGAATTTGGAGTTTAAAAATCAACTTGTGAAAACAAGGTTG
TTTTTGTCTTTATCTTTTGTTAGAGTTATAGATTTATGATTTCATAGGCTTGATTCTATGTGAAATATCTTTTTACTTTTATGCATTTTAATAA
GATTTAAAAATATTTAGATTAAAGCCCCCTTTAATGAGTACAAGAAAAACTCTTGGCTTGTTAGAAGAAAGTATATTCTTTCTAGAATTTGGTG
CAGGAATATGTGTTCATATCCAGGCAAACGGGTGTGTTTTTATCTTCAGACAATGAAACCTTCTCCTCTGGGGCTTTGTTGCCAGGAAGATTAG
AACTAAATTTATTTTTTTCATTTCTGTCATGAAATCATTCCAGATACCTCTTTTCTTCTTTCCAAATGGTTTTCACATGTGTTTGAAATATTTG
TACTTTGAATTGTCGGATTTTCCATGTCCTCCTTTCTCCTTTGTGCCCAGCCTGAGTCAGCACCATCCCGCATTCAGAACCTCCCAGTGAAAGG
GCAGCCTTCATTTTGAGAAGGTGGAAGGTGTTAGGGTTTGGGAGACAGCTCATCCAATCTCCCAAGTCTCATGGTGGATTTGTGACTGTGAGAG
TTTCCGGTTTAAAATCTGAAAAGCCAGATATGCCTGTTTCCTTTTCCCAGCACCATGCCTGTGGAGGGGACAGTCAGACCCAGAGGTCCTTTAC
GTGTGGATGGAGTTCACAGGCGAATAGAGGAGAGGACCAGGGGACGTGGCTTGTCCCTTTTGTCCAACAAAGCATTATATTTTTAAGAATGGCA
GACCTGTTTGCTGAAGTGTTCATAAGATAACAATAGGCTTGAATCTCCAATTCAAATGAATGTCAAAGCACATATCTTTAATATGCTGAATGAA
TATTTATTTTTGTATCCATTAAAACAGTATATTGATCTCTTTTATTCTTTATTAAAATAAAATGCTCTTTTTTAAAGCTGCTGTGTACTGTATT
TTTTTAGTCACTGTCAAATATGATGTAATTCTCACCAGATTCTAGGTTCTGTAAAGGGGGAGTTACTGCTGCAATGTGCAGTGTTAATTCAGTG
CACTGGACTGAAACAATATCGCACTGGGATTTTAAGGATTTTTTTTTTTTTTTGAGACAAAGTCTTGCTCTGTCACCCAGGCTGAAGGGCAGTA
GTACGATCTCGGCTCCCTGCAACTTCCACCTCCCAGGTTCAAGCGATTCTCGTGCTTCAGCCTCCCAAGTAGCTGGGATTACAGGCATACGCTA
CCACATCTGCCTAATTTTTTTTTTAAAATTAGAGTTGTGATTTCACCATGTTGCCCAGGCTGGTATCGAACTTTTGGCCTCATGTGCTCTGCCC
CAACCTCTCCCCCAGCCTCCCAAAGTGCTGGGTTTACAGGTGTGAGCCACCTCACCCAACTCCTGTTTTAAGGATTTTTCTTTTTCAACCTATC
CCTTAAACAACAGCAAAAAGATACGGAGACTTAGACTGATCCTATGGAATGAAAACATTGGAGAATGTTTTTATTCCGAATGGGGGGCTGTTTG
GGTTTCTGTGAGCTGACAGATGAGTGTGCGGAAGGGTGTGGAGGAAGTGGGGTCTTGTGCTGATTTCATCCTCCTGGATGCAGCTGGGGTGATT
TGTACGAGGCGTCCCTGGTTGGGTCAGCCACACGTGGCTGGAGTGTGTGTCCTATTTACGCACATCTCGCTGTGCTAGGCCCTCAGGATACAGC
AAGGACATGAGGGACCCAGGTTGGCAGTCACAGAGATGACTTCCAGCCTAGTAGGACATTCCCATCCATCAACACAACAAAACCCATCAATTCT
GGGAGGTGCAGGGCTGGCCAACCCAGGTGGAGAGAAGTTCCCCAGCAAGCTTCATCCGCAAGGCCATGTCTGTTCTCTCCTCTGCCCTCATACG
AGCCCAGTGAGACTGGGCCTGTTCTTCATTTCTAGCTTACAGATGAGGAAACTGAGGCACAGGCTCATGCGTGGCTGACTGAGAGCCATCTATG
TCCCAGGAGGACAGCTTCGCCTGAGAGGCTTCATGAGCAGATCTCGCTGCCATCCTTGAGCTGGTGGGCTTCCTAACACCCCACGCAAGAGGTC
AGCAGCTGGGTGCGTCGCTCACTGTCTTAGGCATCTTTGCCATGAGTCACACTAATGTCCGCACGCTGCAAGTAGGAATTTTGTTTACAAGCAA
GCTCTTCTCCATCCTGTGGATTCAAGGCCAAATGTGGAAAGTTGATATCGTCTGTTGTCTCCCCAGAGTTCAACGCTCTCGTGCAGACGGTCGA
CAAATCCTTCCTACTCAACTTCTTGCAGCAAGCTCGGGGAGAGGCGGGAGAGGGGAGTGTTTTCATTTGCAGAGGAGGAAATTTGGGAACAGGA
TGCTTCTTTAAGAAGTCGGGTTTCTAGAGAATGCTGTGAGGGCCCTGGATGTGAACTGACTTTTGAGATGCTGACCCCACACTGGCCAGTTAGC
TCTGGGCCCTGGAGGGAAGCTGGTGGGGATGGGCTGACGGGACATCCTGAAAAACAAGCCATCTCCACTCTTGGTAGTTTAAAAATGTTTTAAG
AAGTTAAGGTATTTCAACAATTCCTTTCTGAGTGATTGCCACTTAGTTCATTAACTTTTGCAAAATCCCACTTTGGGAGGCCAAGACAGGTGGG
TCACTTGAGGTCTGGAGTTCGAAACCAGCCTGGCCAACATGATGAAGCTCTGTCTCTACTAAAAACAAAAACAAAAACAAAACACAAAAATTAG
CCAGGTGTGGTGGCAGGCACCTGTAATCCCAGCTACTTGGGAGGTTGAGACAAGAATCACTTGAACCCAGTGGGGTGGAGGTTGCAGTGAGCCG
AGATCACACCACTGCACTCCAGCCTGGGTGACAGAGTGAGACTCTGTCTCAGAAAAAAATGTCAAAATCCATCTGGGACACAACATTTTTGCTT
GCCCTCGTAATGTCCAGAAGGCTGGAGATGATGACCCCTGGGTCCAAGCATCCTCAGGATCCGGTGGGTCCTAGAAATCCTGGGAAAATCTTCC
TGAAAGGGCTTCACCGGGCAGCCACCCATGCCTGCCACAGGGACACTTTCTCTCCATAAAGAAGCAGCTGTGGGAATCAGGAAGAAATGACTCC
TGTGCATGATTCAGACTCTCCATAATAGAAAGTTTTGCAACCCAATGACTCGGAAATGTTTAGAAATTCTAAAGAATAATCTATAATTAATGAC
TGTAATTTTAACAACCCAGTGGGCTGAGAGTGGGGTGTGGCAGTTTCTCAGTGGAAAATCTGGCTGCTTTTACTACAAGTAGGAGGAGAATGAC
AAATATCCTGGCCCCAGGGCACACCTGATCTCAGGCTTTCTCTTCAAGCACACCGTTCATTAATCTGTTAAAAGGCCTAGCTCATGGTAAGAAG
CCCATTTAAACCAGCATTTCCCAAACTTATTTGACTTGACGTTTTGCCTTGCAGCGTGTCTGTGGATGAGAGGTGCCCACAAGCGTTCAGTCTT
TTATCACAATCCGACTGGAACATCTTAGGAGTTGAGAAGAGTGTGTACATTATTTCTTTCCTATTTTCTTTGGGCTACCTTTCAGTCTGGATGG
AGTCTGCCAGGGATGGGATGGCTTCAGTCTCCTCTTTATATGTAACTAACTATTCCTAAACTTAGTGGTATAAAAAAACGCTTTATTTTGTGGG
TCAAAATTTAGACAGGGTACAGTGGTGTTGGCTTTTCTCTGCTCTGCAATGTCTGGAATGTTAGCTGGGAACAGCTGAGAGTGACTTGAAGTCC
TGCAGATAGAACCACCTGGAAGCTTTTTCATTCACATGTCTGGTGCTTGGGCTGGAAGGAGGTGAGGCTGGGCACAGTTGGGATACCGATGAGA
ATATTGACTTTGGCCTCTCTATGGAGTTTGGGCTTCCTCACAATATGGCAGCGTCAGGGCAGTCCCGTGGACACAGGGGAAGCTGAATGGCTGT
TTGTGACCCAGCCATCACTCAACTGGACAAACAAGCCCTCCTAGGTTCAAGAGGAATGGACAGAAACACCACCTCTTGAGGAAGAAGTGTCCAG
GAATTTGCAGCCATGCTTTCAACCTGCCACGGGAGCCCCCGAAGCCTCTCCTTTGCTGCAATCCCACCTGCCTTTTATCCCCCGACCTCTCCTT
GGGCATCCTGATTCCTTTCTGACCCTATTCCTTGAGTCCCAGGCAGATGGGCCTTTTCCTCACTTTGTCTTGGGAGTATGGCAGTGGACTCTGC
CTACTTTCATAATGGAGCCTTTTTCTTGTCCATTTTGATGACCTCTCAGAGCAGGGCTCTGACACGTTACTGGGTTACAAAGACCCTCCCTGCC
AGGGCCAGCGTGCAGCACAGGGTGCTTTTGAGTTCCGGTGAACGGGGTGCTCTGGCTGGAGCCTAGTGCGAAGTTTCCCCTTCTCCGCCCATTC
CTCATCCCTCAGGGCACACAGGTCACCTTTCCTCGCCTTCTCGTCCCCACCTCTAAGGCTTGGCCAAAGTTTCTTCATAAAAGCAAGAAAGATA
ACGAAAGAGCCCCCCATGGGTTATGGTTTTTGTGGGGCCAGGAATGCCCAGTTTGAACCTCCAGTTCCGCCACCTGTAGCCGAGTGAAAGAGGC
TGGGTTATTCAGACACCCCAAGCCTCAGTTTCCCCACTTGTTGTGAGGCTTGAGTGTGCTAACACATCAAGGCAACCCAGAACAGCGCTTGGCA
GAGACTCGCAGTTTATCACTGTGAACTGTCGTTATTGGAAAGTGGGTGGAGTCGGGTTTGGATTCCCCCAGGAGCAGAGACAGAAACAAAAGGA
TTCAAGGTAAGCGGCCTGTATGGGAGGTGATCTCAGAAGTGCTGGGAGGGGAAGGGGACCTGTGGCTTTAATGGTGACCCTGTGCTACTGGAGG
CCTGGGATCTCTGAGGAATGCTGAGGCCCAAGCCCTGCAGAGCCAGCCCCGGGTTGTTAATTACTTGGCACTTTCCTCTTGCCTTATGTGCAGG
GGTGGGCTCAGGCGACTGAAGTCTCAGACACTGCTGCAGGTGCTGGCAGGGGGAGTCACAGCTGCATAAACTGGAATGGGGCAGGTGGGGTGGG
GCGGGTAAGGGGTTGTGTTTGCTTCGGAGAGGGTTTGTCAGCACTTTGAAGGAAAATTTGTGTAGTGGCCGGAACCACAGATGACCCAGGGTTT
GTGTTCCAGCCCAGGGATCATTAGCTGTGTGCGTCACTTAAACTTTCTGTTCCTCAGTTTCTTCATCTGTAAAAAAGGGGCAATAATGTTACCT
ATTGGGAGGATTAAATGACTTAATAAGTATGAGGGGCTTAGAACAGTGCCTGGCACGCAGTACATACTAAGAAAGTGGATTTGCACATATCATT
AATGCCTCCCTCCCTTTCTTTCTTTCTTGTTTATTTATTATTAAATAGTTATAAGTATAAATAATTACAATAATTTATGTCAACGAACTATAGT
GTCAACAATAACTATAGGTAATTATTTAATAATTAGTAATTATAAAATAATTGATACTAAATTAACTATAGAATTATATAGTGCCAGAGTTGTT
TGGCTATCTTTAATTATACATGTAGTTATTTAATTTCACAAATAGTTATATGAATTATGGAAGTGAAGGTACGATGGTATAACTATACAATTAT
TAGATAACTTATCATAGTGGTATACATATTTATCATGCAGTTATTAAATAATTACAATGGAATGACTTATTCGCTCTCTGCGTTTGTGCAAGGG
CAAGGATGGCCACGTCCCAGCTCTATCCAGCCCCTGGCCCAGTGCCTGGGGTTCGGTGGCGCTCAGTTCACGCTTGCAGAGCGAGCGGCGGTGA
GTGGATGCGGTGGGGTCGGGCTGTGGTGGTAAAGGTGGCCGTGAACAGGAGGGGCCACTAGAGCACACAGACGTGAATGACATGGGGCTCCAGG
AGGGGCAGGAAGAGCTACAGAAACAGCATCCCCAAAGCTGCCTGGCACGGAAGAGGTGCTGCAGAAATGGGACTTCCCTTCCCATCCTCTTCTC
CTGATAAACAACTGAAAACATAAGAGCGAGATCTTGGGGCTTCTGTGAGGTTAAATGAACGAACAAAAGTCTTACAGCGGAAATCACTGAGAAT
CTCCCCTCCCCTGTCTTCCAAGGAGAACATGATTGTGAGCCTGCTCCTCCCTTCCTGGGGGGGTTGCTTCCTGCTGGGGAGAGGCTAAGTTGAA
CTCTAGGATCAAGAAAGGGAAGGAGGCAGAGAAAGGTGAGAAAGGTGAGAAAGATGAGAAGGTGCTCGCATGGAGCGTCCGGGCAGTACCAGGC
TGGCCCGAAGGAGGTGCGGATGAGGGGGAAAGGCTGTTTACAGATGTGGTGGCTTCTCCGTGTGCATTCACCTCCTGGGTCTCAACTCGTGCAG
ATGGAGGGTCTGAGTTAATCTGAGGTTACATTTGTTTGCTCAGCTATGCTTTTTCAAGAGGCCATTCCTCTGAGACTGAGCCCCATGGCTCCTG
GCTTTACATGAGTTACACGCATCTTCTTCTCCTTTTTTCTAAATAGTAAGGGGCAGAGAGAGTTAGAAATAAAAAGGAAAGCTGAAAACAAAAA
CCTCTCAGAAGAGACTCTGGCTGTTTTTTGTTTTTTTTTAATTTTTAAGTTTTTATTAATGCTCATCTTCCCTGAAGAGCCCTGGCTGTTTTCT
TGACCAGCTTCTGGAGAGAAAAATCACAGTGCATTTGAGTAGCTCCAGATGTGATCTCCCCATTCAGCATTTCCCTTCCTGTAGCATCTGCCGA
GCCTTCCTTCTGGCTTCTCTGCTCTCCGTGCCTGAAAGGAGACATTGAGCAATCCAGGCCAGTTCGAAATCCTGAGCCCAGAAATAAAGTGACT
GTTGCTTTTGCGTCCTTGCACGTTTGCCCCGGCTCTCAGCAGAGGCAGAAGGTGATGTTTGGAGGAAGCAGATGGCCCGGTCTACTGTACATTT
CAGAACATTTCAACTGCAAGGCTGGCTGGGGCTATTGCATCACTGAGCCCGATAGGGCTGCAGGGCAGAGGCAGGGATCCCTGTGGGTGGAACT
GCTGGGTGGCAGGACGGGGTGGGGGAGCTGAGGGTGGTGTTTGGAGAGGAGGGGGAGAAGGGGGAGACAGTTTGGGGAACGTGCCTGAATTCCT
ACCCAGGTCCAGTGTGGGTTGATGCAAATGGTGCACACGGGCTGAGAGGTCAGCTAGATCTGGGTTCCGATCCCGACTCCTTACTGATGAGTTG
TGTCCTTGGAAAATGACTTCTTCTCTTTGAATTTCAGTGTCCTTGACTATATACATGATGGATTCTTCCCACATCGCATGGATGTTGTGGGGAT
TCATTGTCCTGACCTCCTGGGGTGTTGGTGAGGATTAAATGAAATCATGATTTCGTTTGACATGCTTGGACAAGAGCCCCACACACTAGAAACC
CTGGGTGTGTGTTAGCTGTTGGGAAGATTCTCCTGACCGTGATGATGGGAGAACTCGGCATAGCCCTGCGGGAGGAGTGTTGGTTCTCAACAAC
TGCGGCTGTTTGGGGGTTGTTGGCTTGTACTCCCTCTTTGTAGAGGCTCCTCGCTCCTGGCTCAGGGCTGCTCCCAGGTGCACTTTCGGTGGCA
AGGCATTCAATTGTACACGCTGACTCAGGCGTGGTGAAAGCCAGCTGGCCCTCTGGACCCTGGCCTTGGTCCTCAGCCCTATTGGTTGTCCCCG
CTGCCCGGGATTGGTTGGCCCACGGGGAGGGGCTCTCTGCCTAAGGATGGTCTCCCCAGCTCAGTGACAGAGGGATGCTTAAGCCACGCCACCA
CCTTCGTTCAACTGCCAGCATACCTGAGTGTTTGAGTCAGCCTCTCTCCCTGTGGCCCAGTGACATGGGCAGCAATGTGGGAGGCTGCCCAGTG
GGTGGGGAGTCGATCAGACGGAGCGAAGAGATTTCTCGAAGGCCCTCTTCACCAGCGTAGCTTACGACAGTGCTGGAGCGCCTACCTGAGCAGA
CCACGCTCTAAGCTTGTTTTGCTGAACACCTTCCTATTGTCCCAGAATCCACCTGCTCTCCCAGGAAGACTGATCTCTTCCAGGGACCATAAGC
AGCTTCCCCAACAGGTGCCCCAGCGGGAGACCAGAGGGAGGGAGGAGACAGGTCAGAGTGCCTACTCCCGTTTTCTCCCTGAGTCAACGTTGTT
CCTCCTGAGGCACCGGCCCCCATACCGCCTCCTCCTGGGCTCCAGCAGCCATGCTCTCTCCTGGCCCCCCCTGGTCCCCTGCCTTTAGTGACAG
CTTATCCTCGTTCCTGCTCTGTCTCCTTTGGTTCCCTTAGACCAGGTGTCAGCAAATCTGGCTTGCAGACCGAATCTGGCCACAGAGCTGCAAC
GACAGAATTCAGCGACTGCAGCAGAGGCCGTGCGGCCCCCCAGGCCGAAAATATTCACTATCTGCCTCTTTCCAGCTTTCCAGAGAAAGTCTGT
GACCTCTGTGTGAGACCCACCCACACCCATGAGATTAGTCCCTTTGCAAATCCCTTCCTTGAATTAACCTGTGTTGAGTGTGCCAATGGCTGTC
TGGGACCTACCCGGCACGCATTCTATTATCCTGACGTCCTCTTAGCCTGGCAACATTCTCGTTCTCTCCATTTTGCAGATGGGAAAGCGGAGGC
ACAGGGAGGCTAAATAACTTTGATGAGATTAGGCACACAATCAGTAGGGGGAGGTCAGTTCCAGAGTCAAAGGCAGGTGCCACCAGCACCCCAT
GGCACTTCCTGTCCTGAGCTGCTCTTACTCGCTCTCGGGACGGTCATGCCAAGGAAGTCCCCTGTCCACAGCCGGGACGCCCTGGGTGGTGCTC
GTGGCGGCAGGGCCAGTACTCGAATCAGTGTCCCTACTTTGCTTCTGTGCCCCGGGTCCCCTTGATTGACACACGACCTGTCATCTATTCTGGT
CTCTGAAGGGCCTTTGTGCGCCTCTTCTGTGCTGCACGAGGCGCTGATTTTTACGGCCTGTCCTTTGCAGAGGAAGCTTCTCTCATGAATTTCT
CAATTTTTGTGGCAAGGAGGGTCCCCAAAGCCTGGACATGTCAGCAACACTCCTGTCTTTTCCAAACCTCTCCACGCATCGCCACCAGGAAGCG
GGGCAGCAGGCTGAAGCTGGGACCCTCACTCTGCCCCTTAGAATGTGAGCAAGGGAAAGTCCCAGAGCAGATCCCAGGGCACTACCGGGCCCTT
CCCTCATCTGTAAAGGAGCTCCTTCCTCCCAGGGCCAGAGGAGGCAGCGTGTGGTAAGTGTTGAGCAGGTGTAATTATACATTTAATTACTGCC
CTCACTAAGGAAAGTGTGCAGCTGTGTCCACCGGTGGGTGGTTCTCAACCAGGGGTGGTTTGGCCTTTGGGACATCTGGCCATGTCTGGAGACA
TTTTTGGTTGCCATGATGGAAAGCAGGTGCTACTGGCATCTAGCGGGTCAAGGCCAGGGACGCCGCTCGACACCCCATAGTGTAAAGGTTGGCC
TTCACCTGCCCAGCTAGAAGGATCCAACCCCGAATGTCAGAGGCGCTGGGTTAGAGGAAGGCCGGAGTGGCCGTCTGTCTTTGTGGAGGGAGGG
CCTGTGTCTGTCTCGCTCACTGTCACATCTCCAACACGTAGTGCGGGTTTCACAAATGGTTCTTGACACATGGGCGTGTGCTGGCTGCCCACCG
GAGCTGGTCAACACGGTTCCCACTTCTAAGGCAGGCAGTGACCTGTGCTGGACCTGACACGGGCTTTAGGGGCTCAGAGGATGGGGACGAGGGC
TCCATAGAGTGGGGGGGGTCAGCTCGTCCCTGAT
HUMAN SEQUENCE - mRNA
ATGGATGGGGGAACCGGGCGGCGAGACGGCGGCAGGACGGCGGCAGGATGTGTGACCGGAATGGTGGTCGGCGGCTTCGACAGTGGCTGATCGA
GCAGATTGACAGTAGCATGTATCCAGGACTGATTTGGGAGAATGAGGAGAAGAGCATGTTCCGGATCCCTTGGAAACACGCTGGCAAGCAAGAT
TATAATCAGGAAGTGGATGCCTCCATTTTTAAGGCCTGGGCAGTTTTTAAAGGGAAGTTTAAAGAAGGGGACAAAGCTGAACCAGCCACTTGGA
AGACGAGGTTACGCTGTGCTTTGAATAAGAGCCCAGATTTTGAGGAAGTGACGGACCGGTCCCAACTGGACATTTCCGAGCCATACAAAGTTTA
CCGAATTGTTCCTGAGGAAGAGCAAAAATGCAAACTAGGCGTGGCAACTGCTGGCTGCGTGAATGAAGTTACAGAGATGGAGTGCGGTCGCTCT
GAAATCGACGAGCTGATCAAGGAGCCTTCTGTGGACGATTACATGGGGATGATCAAAAGGAGCCCTTCCCCGCCGGAGGCCTGTCGGAGTCAGC
TCCTTCCAGACTGGTGGGCGCAGCAGCCCAGCACAGGCGTGCCGCTGGTGACGGGGTACACCACCTACGACGCGCACCATTCAGCATTCTCCCA
GATGGTGATCAGCTTCTACTATGGGGGCAAGCTGGTGGGCCAGGCCACCACCACCTGCCCCGAGGGCTGCCGCCTGTCCCTGAGCCAGCCTGGG
CTGCCCGGCACCAAGCTGTATGGGCCCGAGGGCCTGGAGCTGGTGCGCTTCCCGCCGGCCGACGCCATCCCCAGCGAGCGACAGAGGCAGGTGA
CGCGGAAGCTGTTCGGGCACCTGGAGCGCGGGGTGCTGCTGCACAGCAGCCGGCAGGGCGTGTTCGTCAAGCGGCTGTGCCAGGGCCGCGTGTT
CTGCAGCGGCAACGCCGTGGTGTGCAAAGGCAGGCCCAACAAGCTGGAGCGTGATGAGGTGGTCCAGGTCTTCGACACCAGCCAGTTCTTCCGA
GAGCTGCAGCAGTTCTATAACAGCCAGGGCCGGCTTCCTGACGGCAGGGTGGTGCTGTGCTTTGGGGAAGAGTTTCCGGATATGGCCCCCTTGC
GCTCCAAACTCATTCTCGTGCAGATTGAGCAGCTGTATGTCCGGCAACTGGCAGAAGAGGCTGGGAAGAGCTGTGGAGCCGGCTCTGTGATGCA
GGCCCCCGAGGAGCCGCCGCCAGACCAGGTCTTCCGGATGTTTCCAGATATTTGTGCCTCACACCAGAGATCATTTTTCAGAGAAAACCAACAG
ATCACCGTCTAAGTGCGTCGCTTGGGCGCCCCACCCCGTCTGCGTCCTGCATCCATCTCCCTGTTACAGTGGCCCGCATCATGATTAAAGAATG
TGGATCCCTCTGTCTGGGGTGGGATGCCTTACTTTGCACTTAATTTAATAAGGGCATTCTCGGAGGAGTAGACGTTTAATACGAAGTGGCGCAT
AGCCCTGCCGAGATGTCGGTGATGGCCTGATGCG
HUMAN SEQUENCE - CODING
ATGTGTGACCGGAATGGTGGTCGGCGGCTTCGACAGTGGCTGATCGAGCAGATTGACAGTAGCATGTATCCAGGACTGATTTGGGAGAATGAGG
AGAAGAGCATGTTCCGGATCCCTTGGAAACACGCTGGCAAGCAAGATTATAATCAGGAAGTGGATGCCTCCATTTTTAAGGCCTGGGCAGTTTT
TAAAGGGAAGTTTAAAGAAGGGGACAAAGCTGAACCAGCCACTTGGAAGACGAGGTTACGCTGTGCTTTGAATAAGAGCCCAGATTTTGAGGAA
GTGACGGACCGGTCCCAACTGGACATTTCCGAGCCATACAAAGTTTACCGAATTGTTCCTGAGGAAGAGCAAAAATGCAAACTAGGCGTGGCAA
CTGCTGGCTGCGTGAATGAAGTTACAGAGATGGAGTGCGGTCGCTCTGAAATCGACGAGCTGATCAAGGAGCCTTCTGTGGACGATTACATGGG
GATGATCAAAAGGAGCCCTTCCCCGCCGGAGGCCTGTCGGAGTCAGCTCCTTCCAGACTGGTGGGCGCAGCAGCCCAGCACAGGCGTGCCGCTG
GTGACGGGGTACACCACCTACGACGCGCACCATTCAGCATTCTCCCAGATGGTGATCAGCTTCTACTATGGGGGCAAGCTGGTGGGCCAGGCCA
CCACCACCTGCCCCGAGGGCTGCCGCCTGTCCCTGAGCCAGCCTGGGCTGCCCGGCACCAAGCTGTATGGGCCCGAGGGCCTGGAGCTGGTGCG
CTTCCCGCCGGCCGACGCCATCCCCAGCGAGCGACAGAGGCAGGTGACGCGGAAGCTGTTCGGGCACCTGGAGCGCGGGGTGCTGCTGCACAGC
AGCCGGCAGGGCGTGTTCGTCAAGCGGCTGTGCCAGGGCCGCGTGTTCTGCAGCGGCAACGCCGTGGTGTGCAAAGGCAGGCCCAACAAGCTGG
AGCGTGATGAGGTGGTCCAGGTCTTCGACACCAGCCAGTTCTTCCGAGAGCTGCAGCAGTTCTATAACAGCCAGGGCCGGCTTCCTGACGGCAG
GGTGGTGCTGTGCTTTGGGGAAGAGTTTCCGGATATGGCCCCCTTGCGCTCCAAACTCATTCTCGTGCAGATTGAGCAGCTGTATGTCCGGCAA
CTGGCAGAAGAGGCTGGGAAGAGCTGTGGAGCCGGCTCTGTGATGCAGGCCCCCGAGGAGCCGCCGCCAGACCAGGTCTTCCGGATGTTTCCAG
ATATTTGTGCCTCACACCAGAGATCATTTTTCAGAGAAAACCAACAGATCACCGTCTAA

TABLE 17

MOUSE NOMENCLATURE
ICSGNM Bmi1
Celera
HUMAN NOMENCLATURE
HGNC BMI1
Celera hCG23091
MOUSE SEQUENCE—GENOMIC
TAAACCAACCTCGTATTAAAAAAAAAAAAAAAAGAAAAGAAAGAAAGAAAAAGAAAAGGAGAAAGTTAAACTTTATACAGGATTTGTCTTTGAA
GTTATGAGTCCCTAAAGAAGTAATTCATTTATACTAAAGATCTTGCCATTCCTAACCGTACTTTAGATGAGTATCTCGTATTTATTTACGAAGA
CTCTATTGAGTAATTAAAGCCACGGACGTAGTCAGTTTAGGAGCACAGAATTTATATGACAGAGTGCTTTCGAAAGGTATTTATTCGCAGTAGC
ATATAAAATTTTTTAATAATCCTGTCAAATATGTTCATGAATAATTTATTAATCTCCCAATTCTTAACTACCGGGCGTGCACACACCCGCCTGT
GTCCTGCACATCACGCAGAAATCTACTGCCATGTTCCTGAAGTGACAAAGACAAGGTAAAACTGGTATATTTAAGATGTCCTCAGCCTCACAAA
AGGGCAGCGCTGCGGGTCAGGCTGGCGAGTTAACACACGCACTCAGTCTCCCGTTGTCCCGAAGCCCAGCTTCTGGCCACACTCGCGGCGGCGG
GGTTCCCGCCCCTCCCTGCACCAGGCCCAGTGGGGACACAGCTGGTGCGGTCCCAGGTGAGCCCCAAGCGGGACCAATGGGCGTCCGCGCTCGC
CGCGGCCGAGGCCAGCGCCTCCCAGACCGTGCGACAGCCCCGCGCGTCCCGCCCGAGGCCCGCAGCCCGGCTGAGACGAGAGGCCCAGCCGCCG
CCCGAGCTTCACCCGAACCCTCCCGAACNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNACTTGGACAACAAGTATGAAAGTGGAAGCTGTTCTTAAAGCCCCCCCCCCC
CCCCAATTAACCACTACCTGGTTTATCTCGAAGCTGTGTGTGTGTGTGTGAGAGAGAGAGAGAGAGCGAGGAGAGGTGCGGGGACTTGAAGATA
TTAATGGGATTTTGATATTTCTGTTGTGCATGCTAATGTATGCCCTCCTATCAAGCTTCTTTGATGTATTTATGTTCATGGAGTCAATTGCTCT
AAAATGTCTTTTGAAAGTTGTTCATTTTGAAATTTACTGTGTAAACTACCTTCACTTTCTCCTTTGCTTTCACTCCATCCAGCAACCACTGCCC
CCCCTTTCTTTGAGGTATTTAAAAAAAAAATACTAAGACGAAATAAAAGAAGGTAATTGAAAAGAGAGTGACAGCAUATTATTGATGTATTCTC
TTTATTGTTTTGCAGTTAACCTTGTTCATCTCATCCATTTACAATTGAATTTTTGTTATATTTTCAAAAGAGGAAGTTTTACCAAAACCAAATA
TTCCTCTTAGGAACAGGCGCCTAAAGGATTTGTAATCATTAGCAATTAATTTATTTTAGATCACCCATACTTGAAACAGATCGACCCAGTCGTT
TTAAAGCATTGTCATGCAGCAGCTGCAACTTGCATCCTGGAGGCAGGAAAGCACCAAGTTGACAAGTCTACTCTAAGGTACAGACTTTATCTAA
TGCCCGCTTACTTTGAAGTGGTATCTTTGTCATTGTAATTTCACTTTTTAAAGATAGAGACTAAACCTTGCTTTATTTCCCCCCCCTCCTGCCC
CCTTCAGCACTTATTTAGAAGACTGTAAATTTGACCGAGAGCGAATAGAACTGTTTTGCACCGAGTATCAGGTTACTTACTTTAAACTTTTAAA
TTAACAATTAGTACCCACCCCCTTTTTTCCTGCTGTTTGCAAAACTGTGTTTTCTTTCTCCTTCCCCAGAACAATAAGAATTCTCTAGAAACCC
TACTGGGAACGTAGGTACTGTGTTAGGTGTCAAGCTGCGGAGCTTTTCCCTTGTGGGTAACGAATGGAGTGTGCTGATGGGAAACAAAACTGAG
CCAAAAAAATTGGGATCCTGACCCAAAATAAAGGGCCAAAGGGCAAGTGGAATAATTGAAGTTAAGCTAATGTTTTTTTCAAGATAGAGCCTGA
CATAATCTTAGTAGGTGTGCTTTCACTCTTTTGAACATGAAGATGAGGTTCAACAGTCTTTAAGTTCAGAGTGGAATGAAACGAGTTAACAAGA
GAAAGACACCATTGAGAAAATCCTGAAAGTTTTTCTGTAGATACTACAAAATTATCATTGTCTTTCAGTATAGGCAGATCTCTCCCTCATATAA
CTGATGTTTCTTGGCGCTTGGAGTACCAGATAAAGGTAAAGATAACAAGCTTTTCTGTAGAGGGAGTTAGAGGAACTTTTTTTTAAAAAGGTTA
GAGTTAAAGACTGATGTTTTGTGTTGTTTTAGACCAACCAACTTCATAAGATGTACCGACCTGGATATTTGGTCACCTTAAATGTAGAGGTATG
TATAGATCCTTGTCTAAAAATTATGTGTAATAAAAATTTTCATTTCCTTTATATAAACAAACATTTTTTTCTTCCATAGAACAATGATTCCCAA
TCCTACCCAGAGATTAATTTTAGTTGCAACATGGAACAGTTACAGGTACAGTATTAGGACCTTTAAATGTTTCTTTCAGTGGGCATTTGAGAAC
TGTTATGGAGACTTTATAATATTCCCAGCATCAGCTAATGAAGGTTGGTTTAGGTTTTAATGGCAAAGAGAGAATGGAAAGCAGAAATTTAACA
TAGATTTCACTTTTAAAAGTAACATAGTAACATCCCCAGTAGCCTGCATTTACTGGTGCCCCTAATTGAAAAATAACTTTAAAGTTTGAGTTCA
GCACACTTTCTTTCATTCCATCATGCATCTGACTCATAAAACTCAGCAAAAGTAAGAGATTGGAGAATGCCTTTCACAAATTAAACTTCCAAAG
TTTTAAGCAGGAGTTTTAAATTACCCTATAAAGGGAAAGTTCACACAAGTTCTTAAAACTGTTAATATTTTCAGTGTAAAACTGAAGAAATTAA
AGTCTTCGTTGATTGATGGATATGCTAACGAATTTCTTACCAATTAAATAAAATGAAGATCATTCCTGAAAATTTGAATGAAATTAAATGTTCC
AAAGACATCTGTAGTCACTTTACAGTAAATATTAGCGAAACTTTAAACAGAAAATGTGTGTGAATCATAACCTTCCTTAGACAACTTGAGAGAT
AATTTAGATATTCTCAGCCTGCAAAAATTCTCTTAGAAATTCCCTTTTAACTAGAACTTGAATACCTGATTGGAGGACCAGATCCAGCAGAAGA
AGCGCTTCTTTTCCAAAGGGCTTTGCGAGCTGTTGAGCTCCCGTGGACTCCTTAGGTGTTCTTCCTTCCGACTGTGTTCTTGCAGCTGCTTCAG
AGTTGCTGCTTCCTCACTTGTATGCCCAAGTTGTGACATCATTGTACATCTTTTAGGACTTCGTGGGGAAACTTAAGGATGCTTCAAAAAGCCT
GGAAAGAGCAACTCAGTTGTAATTTGGGGAGGCTGATGACCCCAGGAGCCTAGAGGAAAACCAGACGCGCCTTGTCTCCTGCTGGACCATCGTT
CGCGCGACCTGGATGTCATTTTCAGTGCAAAAAGAGCTAGCACCCTTTTGAAGTGATGCCAACCATCAATTTTGACACTTTTTTTACACATGTG
AAATGGTTAAGAGGGAGACTCTTAAGCATCTGGATTTTCAGTATTACTCTTGCGTTCCTTCTATCTTGAGCGTCTTCAAGCCCTATTAAAGTAT
CGTACCCTGAATAAATACGGTGGTATTCCCACAAGAAGGGCGGCATTCTGTTTGGAGTTTGTGTGTGACTTTGACGACTGAGTGTGCCAAGCAC
ATCTGCCCTGCGTGGGCTCGGACGCCTGCCTTGCCCCAGGTTAAGTGTTTNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNCACTTGTGTCTCCAGCCTGCGCGGCTCCCGCGGCCGCTGCGCCCTTTCTTG
AGAGGTAGACTTTGCTGAAACGGGGACGGGATCCCGAGATCGGGCGCTGCCTGGGTCCAGTGGCTTCCCTCCTCTCCCGAAGAAAGGGCTCTGG
AGGTCTTTGAACCTCCAGCGAACGCCCCCATTTTATACGCATCGGTACTGTGGGGAACACCCATCCCGACGAGGTGGGTGTTACCCTCCGCGGA
GCCCCGACAAAAATGTTATTAACTTAACTTGGAGGGACACTTGTGTGAAATTGGATGGCTGGTAAGATGGCGGTTCCCAGTTGTTTCCAAGGTC
TTGTGCATTTGTGTTGGAAAGGGTAAAGTTTTCAAGCTGGTATTTGTTGGGAGTCGACGACAGGGGTTCCATACGTACTTGGGGCGAGCTCTCA
AGGAGGTGGGCATTTAAAATTTGAGCTTTGTTTTGGAGTGAAGCCCGTCACTGTACGTTAGCCTTGCTGAGCGCCCTTTCTCTCTCCACTCTCA
CCCCTCCTTTTTTCTTCTTTTCTCTCGAAGACACTACAACTTTCCCCTTTGCTGTGGGATGAGCTGAGACTTAGGTCCATTGCATTTTATAGAT
TTTGCTAAATGTCAGAATAGGTCATGAAGTAATGGGATGGATGCTTCCGAGAAGGAGCACTAACGCGTTGATTGCTTATGTTTACGTAGTCATT
TGGACTTGACAGCGCTGGAGCATCATCGTTTCCCCAGAGCTTTACAGTTAATAATTGGAAATAAACAAGGACAGAGACTTTGATCATTCCCAAT
TGCAATAAAAGGATTTAGTGGGCTTAGTGGTCACGGTTCAATATCTGAAAGAGAATTGAGCATTAACCCCTTCGCTTTAAAGTAGGCAAAATTC
CGGGCATATTTACCCTAATCCAGATGTGTGGTGGCAGTTCAAGCTATTTTTATAGGAACTCCCCCAACCTTTAGCGAGACGTTGTAAAACGTAG
TAAAGACTTGATACTAGAGATTGTAAATTGTTGACCACATGAGTAAAACCAAGTTCTGACACTATGTACATTTGGGTCCTAAGTACACTAGGGA
GATAAACGACATTTAATCAAGAGGTTCATTTTTCTGTAAAATTATTATTATATGATGTGTTTATAATCTACAGTAAATTGTATTTATTTCCATG
TCTTCTATGAAAGQGAAATAATTTACAATAATCTATGAGGGCAAATTATACTCTTGTAATAATATCTTTAAAACTATCTAAAAGATTGTTTATG
CATGTGTAGACACTCAAATATAATCACATACAAACTTTTGCAAGCCATGTAAGCATTAGGATATATTTGATGTCCAGTCTACCACTAAGCTCAT
GAGCGTATGTTAAGTGTTCAAGTAGGCAAAGTTCTCCCCATCAGTCAAGTCGCCCCCAAGTTTACCCACGCTTTCCTTTGAGCAGTGAGCCCCA
AGTGGGATTATAGAGATCATTTGCATTTCTTAGGTTGCAGTGATCAAGCTGATTGTTTATCATCAGAGCTTGCTTCTGTTTTCCTTCATATTGA
CATTACTGTCCTTAGGCCTCCAACTAAAAACTGACAGCTCCAACTGACTAAGCTGTAGCTTGACTATTGAGTATTTTCAGTGTTTTATGCTTGT
TGCATTTTTATGATTTATAGGGAGTTTCTATGGTGCAAGACGTTGTTTCTTAAGTCTGACCAAGCATTCTAGAAAGTCTCCATGAGGTTGTCAG
TACTGTCCAATAGGATAAAAGAATGAAGAATTGATATTTGTATCAATAACAAAGTAAGAATTGATTAGCATTATGAATGGTTCTAGGAATAGGG
CCAGATTATTTTTATTTGAGGGAGGACAGTTGTGTGAATTCCAATTTAAAGTGAATTTGAGAGACTTTGATCAAGTTCATGTGACTGAAATGTT
CAAAGAGAACTAACTAGTGGTCACCTTCTATTGAGGTTTGACTGCTCTTTTGCGTTAGTGTTTGCAATTTTATAATAGAATGTAACATGGAGAG
GAGGAGGCAAGATAGAGAGAGCCCTTTGGTGGGAACTTGATGATGTGTGAGTATTGGATATGGTTTCTTTCATATGCAGAGCATCAGCCTCACT
CTTTTTAGGAAAGAGCCTGTGGTTTCACAAGAGAGTTGCCTTTAATCACTAAGTATGGTTGAATTTACAGCTTGCTCCTTTTGAGAGTCATAAT
TTCATTTACTCCTTCTCAGCCTGCTTTCCTTGGATTGATGCTACTTGTTGACAGAATTCCGAACAAGTAATCCGGAGCAGCTCTGATTGAAGTC
ACTAGTTTCCCCTTCAGTCACTTTCAGAGAGGCTTGGTTTGGGTTTGGACCATCTTTAGTACCCATTCCTGTTAATTGCAGCCATTGGGGCATG
CTGGTTGCTAGTTCCCTCTTGTGTATTAAATTTTCCTTAGAATAAATCTCCCAGGTGCATCTAATTGCGAATTATGCAAATTCCTAGATTTTGC
TTACAGCAACTTTGGGGCTTAACTTTTGTCAGGTGTAGAAGGTCACCCTGGCTTTAGATTCTGTGAGGAATAAAACGGACATCCCACAGCTACA
TCACCTGAGGTACTTTCTGGTGTGGCATTGCTGACAGGTCTCAACTGTCAGTTTCTAAAATGTATCTGCTGGGATTATCCTTTCATCTTGTGAT
ACTGTATGGGACTTAGGTGAGAAAAGACATTTGCCCTTCCCCCCATCTTTACTCTGCATTTAGTTATGGGCATGGGCTCGTCTCATGCCTCTTT
GTACCTTGTTACTATACCACCCACAAAAGTAGTTTTTCTTGCCTCTAGCATAGTTTATTCAAATATTCAGTTCAGTATTCAGCCCTTCCAAAAA
CTTTCCATGGCTCATCCAATCTACTCGAGGACTTATTAGAGAAGGGAGCCTTAGAAGTTCAGTTCAATAAACGTCTCTGCAACTCCAGTCTTGC
TGGGCAGGGGCTAAGTGTACAGTTGGCAATTCTCAAAAGATCTCTAAGTAAAATTTCTGACATCGCGATTGATAACAGGACAGTCACAAAAAGC
CAAGATGAGAAAGTAATTCTTCCCCCCCAATGGTCATGAAAGAGATCCGGAGAGTGCCTGGAATGGCCGGTGCACTCTTTTTGGGGTTGGGACT
CAGCTGCCGGTCACGCGAGGATCGGCACCCTGCTTCCCTGCCCCGCCCCTCCCCTCCCCTCCCCTCCCCCGCGTCCGCCACCCCGCACGCCCGC
AGAGGCTGGGCTCGGCCAGCTCAGCGCAGAGTTGCAGAGGTGGCCGGATGCCAAGTGTAAGTGTAAGTTGCTATGGAAACCCCAACGGAGGCGA
CTTCCGAATCCGGAACAAGACCTAGCCCGCGGCACTTCGGGATCCGCCCCTCACATCCCCGCTCGTGTCCGCGGTGCCCAGGCCTCAGCCGAGG
CCCACGAGGTGCTTCTTTCCTCCCTACCCAGTCCGGAAATGCCGGGGATGCAGGAGGCTCACTGAGCGCTTTCCCTTTGTGGGGCCTGAGCCCT
GCCAGGTTCTGGTTGCTTGAGCCGTCGAAACAAGAATCACGAAGTTTTTGACTAGAGTGCAGGAAATTCCACAGTAAATTCGGAGACTTAGATC
AATAGTGGCTACTGAATATCTGTGGCGGCGGCCTCGTGGTAGCCGCAGCAGAGCTGCGTGGTGTCTGCAGAGCTCGAGGACAGCTGAAGAGAGA
CAGAGCAGAATCTACTCAGTTCTCAGCCCCAGCCCCCAGCCCTTTTTATCTCGCTCCAGGAATGACCCGAAAGGCCAGAAGTAAATTTAGTTGG
ATCCTTTTACACCATTTAACAGTGGAGGTTATTCTGATCTCATTAGTAAATTCCTTTTGTGGAAAACGTTTAGAGAATCCAGCTGTCCAGTGTT
AAATGAGTTTTATAAACCGCTCAGCATTCAGGGTATACAGTTACTTCCACTTGTGGAGATAACTCAATTCTCGTTTTTTAATTATAGCCAATAC
TTTTTTGTGTCTTGCAGGATCTTTTATCAAGCAGAAATGCATCGAACAACCAGAATCAAGATCACTCAGCTAAATCCCCACTTAATGTGTGTCC
TGTGTGGAGGGTACTTCATTGATGCCACTACCATAATAGAATGTCTACATTCCTGTAAGTACACAGTGTTCGCCTTCTTGGGTCTGTGCATTTC
ACAGCTCATCCATTAAAACCAACACCATTTCCACATTCCTTGAAAATGTTAGTTCATAACTGTGAGGTTTACTTTCTTTTATATGTAGTGATTT
GTTACAGTTAGGCAGTATGTAGTTTTCAAGTTATTAATCGGTTTCACTTTTCTACTTTGAGAAAACCTAGTCATTTCCCATCCTTGAATTTTCA
GTTCTGATACAATTAACCTTTGAACCTTTACTTTCTAAACTCACAAAACCTTTTTATGTTCCTATTTCTAGTCTGTAAAACATGTATTGTACGT
TACTTGGAGACCAGCAAGTATTGTCCTATTTGTGATGTCCAGGTTCACAAAACCAGACCACTCCTGAACATAAGGTAGGAAGTGCTGTTGTCCC
TGGTGGGAAGTGCTGCTAGCATTCCTGGTTTTGCTTTGCAGGACGTTTATTTCTGATATGTGTGCTTTATAGGGGGTAGAACTTTAATTTCTTG
TTATTTTAATTACCCTGTTTAGACAAAGTTTGGTTTTGTTCAACTATTTAAAAGGTGTCTGTCTTTTAAAACTGAGTTAGTTTCCCTTTTTTGT
GCATAACTTTATTGACTTAATAACAGGCAAGAAGAGATTTTTTTCCCCAACATTTCCTTATTCTTTATTCTTGCTAGTGAGAGAATTCTCTGTT
ACATGAAAGGCCTACCTCTCTTTACTCTTGGATGTGAAGAGTTAATTACTGCAGGGCATCTGCAGGGTGGAGAACGAGCCACAGCTCTCCTCTC
TCCTCACAGGACACTCTTACACTCCACCACAACACCTCATCACTGTGCCTCAACACCAGCATGCCTACCCAAACCTTAGAACAGTTCTAGGTTT
GCCATATTAACTGTTTCTTACTTTTTCAGGTCAGATAAAACTCTTCAAGATATTGTATACAATTTAGTCCCAGGGCTTTTCAAAAGTGAGTAAA
TGGATTCACTGAAGTTTTTAAAGTGTTCGTTTTACCTATTATAGCAAATTCTACATTTCCCTTCTCACCCATTTCAGATGAGATGAAGAGAAGA
AGAGATTTTTATGCAGCTCACCCGTCAGCTGATGGTAAATTTTGGGGGGCAAAAGACATTTTATGTCAGGAAGAAAATTATTTAGTAATTTAAA
TTATTTTGTTTAAATTCTCCCTTCTATTCTCTTATACGTAAAATATTCAGTTTCTAAAATTGACTTTCAAACTGTCTTTATAGCTGCCAATGGC
TCCAATGAAGACCGACGAGAAGTTGCAGATGAGCAGAAGAGGATTATAACTGATCATGAGATAATAAGCTTGTCTATTGACTTCTTTGATCAGA
GCAGGTGAGTGCTCCTACAAAGCTACTGTGTGCTAATGTAGTTGCTAGGTACTTTGTATCACTTTGTATTTGTATTTTCTCTCTCTTAGATTGC
ATCGGAAAGTAAATAAAGAGAAGCCTAAGGAAGAGGTATGTTCTACTTGGGAGTTTAAGTTGTCCTTTAGAATATTAAGGACTTATATGATATT
ATTAAATGGCCAAGTTGTGTTAGGAGTTTGTTGAGTTAGTATTTTTAATACTAAGTTCAAAATGTAACTAGTTGAGAGTTGTTGCATATTGGTG
TGACTATTAGTATCTTTTTATCATATTTAGGCCATTCAGACTTTTTTGTTTTACCTGAAAGTTAGGAATTGGAAGAAAATTACTTTTGTAAATG
TGCTTCTAGGTGAATGATAAAAGGTACTTACGATGCCCAGCAGCAATGACTGTCATCCACTTGAGAAAGTTTCTCACCACTAAAATGGACATAC
CCAATACTTTCCAGGTACCTACCTCATGCATTTTGTGCAGATTTTCAAATTTAAATGTTTTTTTTTTTTCATTCTGGACCTTAAGAAATTTTAA
ATTTGGTAGCAAAGTTTAAGATACCTCTTAAAATGGTTCGTGAAGATGCTTTTTAATCGTTTTCGGTTAGATCTTGACGACAGAAACATACATA
TTTATCTTTGTTCTTTTGCCACTGAATACTTCAAATATATTTCAATAGATTGACGTCATGTATGAAGAGGAACCTTTAAAGGATTACTACACGC
TAATGGACATTGCCTACATTTATACCTGGAGAAGAGTAAGTAGCTCGTTACCTGGAGGCTATGTTGGCCCAGTACATATAAAGAATTGAGTGGT
TTTTCTTTTTCTTTTTAATGTTGTAGAATGGCCCACTACCTTTGAAATACAGAGTTCGGCCAACTTGCAAAAGAATGAAGATCAGTCACCAGAC
GGATGGACTGACGAATGCTGGAGAGCTGGAAAGTGACTCTGGGAGTGACAAGGCCAACAGCCCAGCCGGAGGTGTTCCCTCCACCTCTTCCTGT
TTGCCCAGTCCCAGCACTCCAGTGCAGTCTCCTCACCCACAGTTCCCTCACATTTCCAGTACCATGAATGGAACCAGCAACAGCCCCAGTGCTA
ACCACCAATCTTCCTTTGCCAGTAGACCTCGAAAATCATCACTAAATGGCTCATCAGCAACTTCATCTGGTTAGGACTGTTAAGGAAAAGATTT
TTCAACCCCCTGATTTAGATACCTTCATTCATTACAGCTTTATAGATGCTAAATACATGTGACTGTCGTCCAGTTTGCTTCCTTTTGTAGTGAC
TTTAAATTTGGCCATAAATCATGGACTAGATCTGATACTTCATATGTATGTTAAGTGGAAAGATTGATTCTTTCTCTAAAGAATTGGATTCTGA
GAAGGATTCTGTGTTAGGAAAGATGTGAAATGATTTCTGTGACCACTGTTTGGATCTGGAAATGTTCTACAGTGGGTAGACATTGGGCCATAGT
TTGTTAATCTCAATTAATGCCTACATTACATTCTCTTTGATTCTTCTTCTTATTATGCTGTTCTGTGAACCTGTAGAAAACAACTCCTTTTTAT
CTTGAAATTCAGCAAATGGAAAGAATAAGCATAGAATACTCCATTCTGTGCAGCCACGTCACTGTGAATAACAATTTCTTGCATATTTAGCCAT
TTTAATTCCTGTTTGATTTTTACTTCTCTGTTGCTACACAAAATGATCAAAGGAAACTTGTTTTACAATCTGTATGCCTAAAAAGCGGCTACTA
CCGTTTATTTTACTCACTTGTTGAAATGATTCACTTTTGTAAGAATCACATCCCATTATGCTTGTTGTACAATGCCATATTGGTATATGACATA
ACAGGAAACAGTATTGTATGATATATTTATAAATACTATGAAAATATTGTGTTTCATGCATTCAAAAACAGTTGTAAACTTCTCCAAATGGGTT
CGACCTTTGCGGATATCACAGTGCACCCAATGCTGAGGCTTACCACCAGCACAGAATTTGTTTTAATGTTGATAACCAGTTTCAAATTCTTTTT
CACTTAGAAGCAAATGGCAGATCTTCATAGTTCTGGCTTTTCTCCAATAAAACTTTCAAAATTAATATTGCAGTAATGAAAAATTTTAACCAAG
GCAAATATAGTGTTTTAATTTTCCATTGGTTAAAATAGAATTAATGTTTTTAAATTCTGTTTATCAGTGGAATTCTAAAATAGATTTTAAAGTC
AACCAGCTAATTGCTGAAGTAAACCATTTTATCTACAATTTTAATTTAGTGTATGTGGCACAAGTGTAATTTCAAATTTAACAGTTGTTTTCAT
AGTTAAAGGAATCTGCCTTCACTGTAACGTCTGGAAACAGAAACAATTTGTTATGATTTCTACAGGGATTTTTAATAGAGCAAACATGTTGAAT
TTAAAATATGAATCACCCCTCCAAGTCTCAGATTTGCTTTGGTCGAACTTAGCGTGTGTTTATCAGCCATCAGTTATTTGTGAGGATGTTAATT
CCGTATGAATATTGTTTCATGTTTGTATGGGAAACTCTAGCTAAGCATTTCATTGTCTGCAGTCTGCAGAAGAAGCACAATTGTATTGCTTTCT
CTTGCTTGTAGCCATTAAATCATTACTTTTACACACATTGCTGTTAACTTCTGCTTTCTTTAAACATTTAGTAAATGATGTTTTATGAAGCCAC
AAGATACATATATTTTTATTTTGACCTAAATTTCTACACTCCCATTGTGTGTGTGTCATTTCTAATTATAGATGTAAAATTAAATTTCATTTTT
AATTGGAAAATTCAATAAAAAGATATTCATTTAGAAAATACTATGCTCTTTAATTAAAATTTTGCTATCAAAACCACAGTCTGCAGAAGTTTTG
GGAACCCTGTAGTGGATTGTAAGAGCTAAAACCTAAGGAAAATACTTTGTTGTCTTCAAATGATGTTGTTAGTAAATGCTTTGTTGTATATTGC
TCGAAAGCCCAATGAAAGAAGTATCTGCTTATTTACAATCTTTGAAGCAAAAQTrACCAATGTTTGCCAATTTTGGTGAATTTAACTCATTTTT
CTAGACTTTTCTCCAGGTTTTCATGGTGTTACCTAAGACAAAAGACATCTCACCCTCTATGATGGACTTACTTCTGAGAGTGCGTTTGAGGCAC
TTATGGCTTACTAAGCAGTGTGTCACCATACTTGAAAACACTTCCATTTATTGTATCTGGGATGAGGCTTTTTACCCTTACTCAATTTGAAAAT
TGCTTAAGCTTAAATGATATTTCAGTCAAAATTTGTCTTTTAATAAAACAACAGAAAGATGCATCGTGGTTTGCTTCTTAAATGACCAGTCATT
GCTGCTCTTACCGTTAAGATTTAAAACCTTTCCCAACTTTTTAAAATCTTCACCTTAAGAAAGCCCCAATGGAATCTCCTTTGTAATTTACCAC
ATTAGGGTACGTCAAAAGTTGGCTTTAAAGTTTGACTTTCACTCCTAAAGGTATTCTAGCATCCTAGAATACAAAGATAATAACCAAGTAGTCT
ACAGAAAATGCCTAAATTGATCTCACAGATACTAGTTTAAAAGTAGAAAGAGAAATGGGGCCCACTGCTGTTGTGTAAATAGTGATCTATCTTG
CTTTACACATGCATAATACTTTACAAGTACTTGCACCTCCTAAATTGCCGGCCGTTAATTGCCCTGAGTAGAAAGGGGTTGCCCTTCAGTTGAC
TTAACTAGTTAAGCACATTGCCTCATTTCCATATGCGCATGTTCAGATGTTACGGTCTCTGGAAGTAAAGTACAACAGAAGCAAAGCTTTCCTT
CATCTTTTCCTGTTTTCCCAAGCGTTGGAATTAAGCTCCTGGTGACTTCTTTCATTGCACTGTACTCGTTGTGTGGGCCCATCAAGAACAAATT
CTCTTCTGAAACCTTTAAATGCCCACAGGAAATACTCCTTGCAAAGGTCAGCAAGAAGGGAAGACACCGTCTTGTCTGTGTGGTTTATGACACA
GGTTTAGGCGGAATAGGATTCACCTGGCCTGTGGCACCAGGGACCCAGTCAACACCCCCAACAAGTTGTTTTCGCTTTCTGTTCAAAAGATTTA
AGACACTTTTCCTCCCCTTGCTCCAGGAATGACAGAATGACAGAAGTTCTTATAGTGCTTACATGAGACAGACTGAAACAACAAAAGGCCGTAG
TCAAATTGTACTTAAACTAGCTTTCCATCATGTTAGAACCCTTCTCAAAAAGTGCATATCCCTTAATACACCCAATGAAGTGGAACTTGGACCC
CCTGCTCTAATTTACAGCTCTTAGATATATAGCACTTAAGGAAGTTATCTTCTTGTATAAACAGGTGGTTGTTTCCAAAATCTGTCGTCCCCCC
CCCATCCCGACATCTGCAATAGTGGCACCCCCACCCCCAATCCCTTGTTTCCAAGTGTGACCAAGGATTTATGTAAGGAAGAGAAACAAGTTAT
CACACATTCCACCCTCCACTAAGCCTGGTGAGAAACAGTCCGGAGGAGTCCTTGAACCAGACAATCACACACCAAATCCCAAGATCACCCATCT
TTGATTATGAAAGTCTCTGAAAAATGTTTACACAGTCGAGTGTTTCCTGCTTCCGCAGTTAATTTTTAAAACTTTTTTTTTCTTTTCGTTCCTA
AAACGGATTTCCTTTATTTGCATCAGTTTCTTTTTCTTAGTGTGTAATTTCTGGAGGTATTTGTCACACCAAGAACTTCACTAAAACTGCCTTC
ACCAACACAGAAATCATCTATCACTCCAGGATCCTTATGGCATTTCTGAAAACGTTCAAAGTGGAAAGCTCATCTTGGCATCTCATTTAGCCTC
TTGAATAAATAGTAAAACACCCAATAAAAACTTATGAATTAAATTCCCTTCTCTCTGACCCTTGCCAAAAACAAAACAAAACAAAACAAAACAA
ACAAACAAAAAAACAACCAACAATCTACTTCCAGAGATTATCAGAAGAACTTTTTTAAAAAGCTGCTCTTCCCTTGGTTACCTACATGTCAATA
AAAAACATCCCTGGTTGGTATTAACTTGGGGCCATGATGAAACCCAAACTACGAGCTCCAACTCTGTTCTAAATCAGGTAGAAGCCAAGCTGAC
AGGAGTGACTTGACTTAGAAGGCTATGGATCTTGGTATGGGGGAAACACGTCGAAAATTGGGACTAGCAACAGAGATTGCTGCAGTGTTTCTTC
CGAAGGACCCAGAAACCGGGGGAGATATTCCAATAAAAGAAAGTACTAGCGGAAAATAAAGCCAGGGACCAGAATGATTGTCACACCTGTCTCC
TCCACCCCCCCCCCCCCACCAAAAAAAAAAAAGTCTCCAGTTAAAGGATGGAGGGGAGCGGGATGACTGTGGAAGCAGCTTCGCTTGCACGGCA
GCGCCGCGTTCCACGTCAGGGGGAAGGTTTGAGGTACTGTCCCTCTGTCCGTCTGAGCTCTAGCTCTCCCCGCACGCACGAGAGAGCGAGCGCA
GGCCGCGAGGACCCGGGAGGCCATTTTGCGCGTGCGCCGCTCATCTCGCACCGCCCCCAGCTCGGCGGACGCGGCTCCCGCCAAATTTGAATGC
GAGCTCGTCAGGCCGGCCCGCGCTCCCGAGGCCGACGGACCCTGCGGGCTTGCGGAACCGCAGCTCCCCGGGATGGGCCGCCAGAGCCGCGGCG
CTGCAGCCTCGAAACGCCACCTTCTCTCTGAAGCCGTCCACGTCCAACCGCCGCCCCGCCGTTAGGGCGCGGACCGGAGTCCCGCGTGGACCCC
TTTACCCCGAGGCTGCAAGACAGTCACCCCTTTGTCCCCTCCTCTCTGCACGCTTTAGTTGCAAAGCCACTTCCCCTTCACCAAGGTGGTGTCT
TTGGAGCTTGGAGTCCCGTTGTTGTTGGACGGATTTGAAGCAAAGTTACTAACAGACTGGAAGCTGCCCAGGTCCCCCGCAGGTGACCAGGACC
AGGAGTATGGCGTCCTCACTTTGAGCGTGTCCATCTCCTGGGTGCACCCCCTGATGCTGCAGCGGGCGAGGACGACACAGGCGGGGCACCTCGG
GCTCTGGGAGAAGGGTCATCCAAGGGGACACCCAATCTCTACGTAGGTCGTGGAGCCGGCGAGAGGGGCGCACGGATGCGAGGGCGCACGGGCG
CAAGCGCTTCTCTGATCCGGGAGAAGCGAAAGTGGCCTCCCCGCAGGCAAGGCAAACAAACCGATCCGCTAATCAGTGCAGCAGGCTGCAGAAA
CCGCCATTTGGGCACGGATTTCGGCGAGTCTGTAGCACGCTGTGTGGGAACTCATAACTGACTCGCCCAAGAACACCCCCACACACCCCAACAC
ACACCATTTTAGCGAGTAAAAGTTCTCCCCCTGGCTTTCCTCTTCTGGGGCCTGGGAGGGACCTTTCCTCGGACCTTTGTACCCAGGGGCCGGT
GTGACAGAGAATGGAGCCCAGCTGTCCTCTGCCCACTACTGAAGGTCTTATTTCACTGTTGCTGAGGAGCATGTCTAGGTTTCCCGCCCGTGAT
CCCCACCCCAGTCTCCCACCTCACACNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNN
MOUSE SEQUENCE—mRNA
CCCCCACGCGAGTCTGAAATCATAATCGTAGTATTAAAGGCAGAGATCGGGACAGACAATGCCGAGGTTGGCCAGGGAGCCCCGCGCACGCTTA
GCAACACCTCCCGGCCCCGCAGAATAATACGGCTCGGCCCCTCTGCGCGCCCTCCCCCGAGCGCGGCCGGGAGGCGGCGGCTGCGCCGAGGAGG
AGGAGAGGCCCCGAGGAGGCGGCGGCGGAGCCCGCCCCCACCCCCACGAGCCCGCGCGGGAGGAGGAGGCCGCAGCGCGCGAGCACCACCCCCG
CGGGGGAGCGGCATGAGCGGAGCGCGGCGGCCGTGGCTGCTCTCGGCCGCGCTCGGTGCCCATTGACAGCGGCGGCGGCGGCTCGCTCCAAGAT
GGCCGCTTGGCTCGCATTCATTTTATGCTGAACGACTTTTAACTTTCATTGTCTTTTCCGCCCGCTCAGATCGCCTCGCGCCGGCCGCTCTTTC
CGCCATCTTTTATCAAGCAGAAATGCATCGAACAACCAGAATCAAGATCACTGAGCTAAATCCCCACTTAATGTGTGTCCTGTGTGGAGGGTAC
TTCATTGATGCCACTACCATAATAGAATGTCTACATTCCTTCTGTAAAACATGTATTGTACGTTACTTGGAGACCAGCAAGTATTGTCCTATTT
CTGATGTCCAGGTTCACAAAACCAGACCACTCCTGAACATAAGGTCAGATAAAACTCTTCAAGATATTGTATACAAATTAGTCCCAGGGCTTTT
CAAAAATGAGATGAAGAGAAGAAGAGATTTTTATGCAGCTCACCCGTCAGCTGATGCTGCCAATGGCTCCAATGAAGACCGAGGAGAAGTTGCA
GATGAGGAGAAGAGGATTATAACTGATGATGAGATAATAAGCTTGTCTATTGAGTTCTTTGATCAGAGCAGATTGGATCGGAAAGTAAATAAAG
AGAAGCCTAAGGAAGAGGTGAATGATAAAAGGTACTTACGATGCCCAGCAGCAATCACTGTGATGCACTTGACAAAGTTTCTCAGGAGTAAAAT
GGACATACCCAATACTTTCCAGATTCACGTCATGTATGAAGAGGAACCTTTAAAGGATTACTACACGCTAATGGACATTGCCTACATTTATACC
TGGAGAAGAAATCGCCCACTACCTTTGAAATACAGAGTTCGGCCAACTTGCAAAAGAATGAAGATGAGTCACCAGAGGGATGGACTCACGAATG
CTGGAGAGCTGGAAAGTGACTCTGGGAGTGACAAGGCCAACAGCCCAGCCGGAGGTGTTCCCTCCACCTCTTCCTGTTTGCCCAGTCCCAGCAC
TCCAGTGCAGTCTCCTCACCCACAGTTCCCTCACATTTCCAGTACCATGAATGGAACCAGCAACAGCCCCAGTGCTAACCACCAATCTTCCTTT
GCCAGTAGACCTCGAAAATCATCACTAAATGGGTCATCAGCAACTTCATCTGGTTAGGACTGTTAAGGAAAACATTTTTCAACCCCCTGATTTA
GTTACCTTCATTCATTACAGCTTTATAGATGCTTAATACATGTGACTGTCGTCCAGTTTGCTTCCTTTTGTAGTGACTTTAAATTTGGCCATAA
ATGATGGACTAGATGTGATACTTCATATGGATGTTAAGTGGAAAGATTGATTCTTTCTCTAAAGAATTGGATTCTGAGAAGGATTCTGTGTTAG
GAAAGATGTGAAATGATTTCTGTGACCACTGTTTGGATCTGGAAATGTTCTACAGTGGGTAGACATTGGGCCATAGTTTGTTAATCTCAATTAA
TGCCTACATTACATTCTCTTTGATTGTTCTTGTTATTATGCTGTTCTGTGAACCTGTAGAAAACAAGTGCTTTTTATCTTGAAATTCAGCAAAT
GGAAAGAATAAGCATAGAATACTGCATTCTGTGCAGCCACGTCACTGTGAATAACAATTTCTTGCATATTTAGCCATTTTAATTCCTGTTTGAT
TTTTACTTCTCTGTTGCTACACAAAATGATCAAAGGAAACTTGTTTTACAATCTGTATGCCTAAAAAGCGGGTACTACCGTTTATTTTACTGAC
TTGTTGAAATGATTCACTTTTGTAAGAATCAGATGGCATTATGCTTGTTGTACAATGCCATATTGGTATATGACATAACAGGAAACAGTATTGT
ATGATATATTTATAAATACTATGAAAATATTGTGTTTCATGCATTCAAAAACAGTTGTAAACTTCTCCAAATGGGTTCGACCTTTGCGGATATC
ACAGTGCACCCAATGCTGAGGCTTACCACCAGCACAGAATTTGTTTTAATGTTGATAACCAGTTTCAAATTCTTTTCCACTTAGAAGCAAATGG
CAGATCTTCATAGTTCTGGCTTTTCTCCAATAAGACTTTCAAAATTAATATTGCAGTAAAGAAAAATTTTAACCAAGGCAAATATAGTGTTTAA
TTTTCCATTGGTTAAAATAGAATTAATGTTTTTAAATTCTGTTTATCAGTGGAATTCTAAAATAGATTTTAAAGTCAACCAGCTAATTGCTGAA
GTAAAGCATTTTATCTACAATTTTATTTTAGTGTATGTGGCACAAGTGTAATTTCAAATTTAAGAGTTGTTTTCATAGTTAAAGGAATCTGCCT
TCACTGTAACGTCTGGAAACAGAAACAATTTGTTATCATTTCTACAGGGATTTTTAATAGAGCAAACATGTTGAATTTAAAATATGAATGACCC
CTCCAAGTCTCAGTTTTGCTTTGGTCGAACTTAGCGTGTGTTTATCAGCCATCAGTTATTTGTGAGGATGTTAATTCCGTATGAATATTGTTTC
ATGTTTGTATGGGAAACTGTAGCTAAGCATTTCATTGTCTGCAGTCTGCAGAAGAAGCACAATTGTATTGCTTTGTCTTGCTTGTAGCCATTAA
ATCATTACTTTTACACACAAAAAAAAAAAAAAAAAAAA
MOUSE SEQUENCE—CODING
ATGCATCGAACAACCAGAATCAAGATCACTGAGCTAAATCCCCACTTAATGTGTGTCCTGTGTGGAGGGTACTTCATTGATGCCACTACCATAA
TAGAATGTCTACATTCCTTCTGTAAAACATGTATTGTACGTTACTTGGAGACCAGCAAGTATTGTCCTATTTGTGATGTCCAGGTTCACAAAAC
CAGACCACTCCTCAACATAAGGTCAGATAAAACTCTTCAAGATATTGTATACAAATTAGTCCCAGGGCTTTTCAAAAATGAGATGAAGAGAAGA
AGAGATTTTTATGCAGCTCACCCGTCAGCTGATGCTGCCAATGGCTCCAATGAAGACCGAGGAGAAGTTGCAGATGAGGAGAAGAGGATTATAA
CTGATGATGAGATAATAAGCTTGTCTATTGAGTTCTTTGATCAGAGCAGATTGGATCGGAAACTAAATAAAGAGAAGCCTAAGGAAGAGGTGAA
TGATAAAAGGTACTTACCATCCCCAGCAGCAATGACTGTGATGCACTTGACAAACTTTCTCAGGAGTAAAATGGACATACCCAATCATTTCCAG
ATTGACGTCATGTATGAAGAGGAACCTTTAAAGGATTACTACACGCTAATCGACATTGCCTACATTTATACCTGGAGAACAAATGGCCCACTAC
CTTTGAAATACAGAGTTCGGCCAACTTGCAAAAGAATGAAGATGAAGTCACCAGAGGGATGGACTGACGAATGCTGGAGAGCTGGAAAGTGA
HUMAN SEQUENCE—GENOMIC
CAAAAGCAAAATCCAGTTGCCTGAGATCGGAGCTTCCCGCAGGATTCGCGCAGAGCGAGGAGCGGGGAGGACGCCGTTTGCGGGAGGCGGGGGC
CGGCATCCCGACACCGTCCGCAGTCTCCCCGGCCTCTCTCAGGGAACCGAGCTGGTGGCCCTCCTAGGTAAGCGCCGAACCANNNNNNNNNNNN
NNNNNNNNNNNNNNNNNNNNNNNNNNNNNNNNNNNNNNNNNNNNNNNNNNNNNNNNNNNNNNNNNNNNNNCCCCGCCGCCAGCCGCCCCGAGCC
GCGTGCGCGCGCGGCCGCATCAGCTGAGCGCGCGGCGCGTGTCACCTCGTGTGCGTGTCGAAGGTCACGCCCCGCTCACAATGGAGCTCTCCGA
GTCTGTGCAGAAAGGCTTCCAGATGCTGGCGGATCCCCGCTCCTTCGACTCCAACGCCTTCACGCTTCTCCTCCGGGCGGCATTCCAGAGTCTG
CTGGACGCCCAGGCGGACGAGGCCGTGTTAGGTAAGCCGCTCGGCTCGGCTCGGAGCTGGATCCGCCGGGGTGGAGGGGCGCTCGCAGAAGAGG
AGCCGGCGGAGCGAGAAGGGGAAACCCCCGGGAAGAGGAAGTCTCCCCCCTTTGCCCTTCGCTCCGGACGCAGTCTTCGGGACGTGGCTCTGCC
AGGACTCGGAGCAGACTCTGAGCATTGTTTTGGCCCCACTTGGCACCTGCATAGAGCCAACTCCACGTTCCTGGCCCGGTTTCCCTCCCACTCT
CTTTGTTCTTTTGTAAAGCCGGTGTTCAAGTCTTGTTTGTGGCCTAAACATCTAAGGTGTTTAGCCTTGTCTGAGGCTGCGGCTGTTTTGACAG
TTCCACTTCATCTGGGGAGCTCCCTGCAATTCAAGCTTCCAGGGCTAGGCTAGCAAAGGTTTAAGGGTCTGTTTCCGAAGTAATTACTACCTCA
GACAGTGTTGACATTTGCCAACTTAGAGCATACCTCAACTTGCTATGATCACTCCACCGCTCTAGTTCAGCATGTTTACACAGGTTCTACGCCT
AGAACCTTAAACTATGATGCACAATACTTTGTTCCGCCCCAGTTACCAGGATGCGAGTGTCTTTTAAGCGATCCGGTCAGTGACTGCGGAGGTG
CAGTGTCAUTATGGATTGAAGACATATTTTAAGACCGATCGCTTTTACATCATTTTCTTAAATATAAATATCCCAACAATTCAAATTTTTGTTT
TTTTTCTGTAGTTTCATGAACTAAACACTTGTACAAAAAGTATGAAAGTGGAACTTCTTAAACGAAAATAGTAACCACCACCTGGGTTAATCTC
AAAAAAAAAATGACTGTCAAACCCTGAAAGTACTAATATAATTTTCAAATTTGTATCTTATATTCTCAACTCATGCCCTACCTTCTACCCCCTT
GATGTATTTGTTCATGGAGCCGGTTACCCTAAACTCATCATTTTTAGGATGGAAGTTTATTCTAAAATTGACACAATGTAAAATATTTATCTTG
TTTTTCCTTTGAATTTACTCCAGTGTTAGAATGACCATTGCATTGTTATATTTTCTTTGGGGGCTATAATATTTTTTAATAGTAAAAATATTAA
GGCAAAATAAGTTAATTCAACCTACTAATAGTAGAGTTCTTGTTCTGTTCTTTTTATTGTTTTTCACTTGATCTTGTTGCTCATCTCATTTACT
TATAATTGACTGTCCTTATAGTTGATGTTTTTAATCTTGTTAAAGGGTTTTTAAACCCAAATTAAAATATCCTTTTTGGAACATGACTGTAAAG
AATTTATTATCATAGCCATCACTGGAAGTTAATTTATTTTACATCATCCAGACTTGAAACATATCGACCCACTGGTTTTAAAACATTCTCATGC
AGCAGCTGCAACTTACATACTAGAGGCAGGAAAGCACCGAGCTGACAAGTCAACTCTAAGGTACAGGATTTATTTAAATATCCATTTATTTTCA
AATACTACCTTAATGATTTCAATTTCAGTTTTAAAAATGGAGACAGAACTTTGGCTTTTTTTTTCTTTCTTCTACCACTTATCTAGAAGACTCT
AAATTTGACAGAGAGCCAATACAACTGTTTTGCACGGAATATCAGCTTACTTACTTTAAAATTTTTAATTAACAGTACTATTTTTCTTTTACTT
TGTTTGTAAACATGTTTTTTTTTCTTTCTTTCTTGCTTTCTTTTTTCTCTCCAGAATAATAAGAATTCCCTAGAAATCCTACTGGGAAGCTAGG
TACTGTATAAGGTGTCAAGCTCAGGCACTTTTCACTTCCAGGTATGAATGGAGAGTGTTCCTGTCAAACAAGATCTTCCGCATCTTGACCCAAA
GAAAAGGAGCCAAAGCGCATGTCAAGCAATTCAAGTTAAGCTCATGTTTTTAAACATCGTTTATTGACATCATTTTGAAATGCTCCTTTACCAT
TCATAATTTAAAAATAAAGTTTAACAATGGTTTAAATTCAGAATGGATTAAAATGGAGTTCGGGGTGGAAAGTAGAGCCATTCTTAGTAAATAT
AAATAACTGAAAAGTTCTTCTGAGCACACTATGTACGAAGTTATCATTGCATCTTTCAGTATAGGCACATCTCTCCCTCATATAACGGATGTTT
CTTGGCGCTTGGAATATCAGATAAAGGTAAAGTTTAAGAAACTTCTCTAGCGGGGGATTTAGGGAACTTCTTAAAACCTAGAGTTAAAACCTGT
TGCGTGTTTGTTGTGTTATTTTAGACCAATCAACTTCATAGGATGTACAGACCTGCATATTTGCTGACCTTAAGTGTACACGTATTTATAGATA
AGTCTTATCCAATAATGAAATTTATAATTTCATTGATTTAAATAAACAGCGTTTCTTGTTGCGTGTTTTTTTTCCTGCCCTAGAACACTGATTC
CCCATCCTATCCAGAGATTAGTTTTAGTTGCAGCATGGAACAATTACAGGTACAGTATTAGGATCTATAAATATTCCTGTCTTTTTATAAATGT
TTACTTGAGAGTTATACTCTTGGCAAGTTCAAAAGAAAATTAATCTAACCATTTGTCAGCAGATAATCAACATTGGTTAAGGATTTAATGGGAA
AGGGGCATCAAAAGTAGTATTTTAAGATAGGCTTCACTTGAAATTAAGATGTTATTAATAGAGAATTGGTCTGTATTTCCTGTTCTTGCTGATT
GAAATAAACTTCTGTAGTGTTTAGTGTAAATTTCTTCTATTCATGATGAGTTTGGTCCAATAAGATAAACAAAATTAAGAGCATCGCTTTGAAA
ATGTCTTTGCTTTCAGAAATTGAATTAAACTGTCAAAATTCGATTTTGAAACTTTTTAAATCACTTTATAAAGGGGCAAGTATACATAACATTC
TAAAACTGTTTATATTGCCCATTTAAGACTGCCAAAATTCAAAACTACATTTATTAGTGGATATGACAACGAATTTATTTCATAGTTTTTAAAA
AATGAAGCTCATTACTGGAAATTTAAGTGAAATTGAATTTTTCAAAGGCATCTTCTGCAGAGTCGTTTTAATGTAACTATGAATCAAACTTTCA
ACTTTCACAAAATGTGTATACAGCATAACCTTTGTTAGATAACTTGACAAAATTCAGATGTATCTGGCCGCTTAAGGATCCGGTTACAAATCAT
GTTTCAGTGTGAGGGCAAATAACCCATTTGAACATGAGGTATTTCCGAGGAAACGCTTTTTTTGCCCGATAGTAGTTTGTCAAAACTTAAAATC
TTGTTGAATTTTAAACTTGTTTGGAAGGTGTGTCCTCATTTTTTTTCTTGCATCTTTCTTGAGCTTGGATACTTCTTTCCATGTTTTATCCTAA
AAACGTGGTATTGTATACACGTCTTTTAGGANNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNCGCTCAGCCCCGGCGCGCCGCGCGCGGCCCCACGTTTTAGTTCCCCGCTCCCCCGGGCGCCCCTCCCCACCCTCGCGACGCACATGGC
CCCCGAGAGGAGTACTGGTATTTCCAGCCTCGGCCGCTCTCCCGGCCGCAGCGCCCTTTGTTGAGAGGTAGATTTGGATGAAACGGGGACGGGT
TCCTGAAATCGGCACCTGCTTCGGTCAAGTGGCTTCCCTCCTCTCCCGAAGAAAGCCCTCTCCAGGCGCTCCAGCAGCCAGAGAACGCCCCCAT
TTTATAGGGACGGGTTGCGTGGCGGCCGCCCATCCCGCCAAGGTGGGTGTTAGGCTGGAGAGAGCCCCGACGAAAATGTTATTAACTTAAGTTG
GGGGGACACTTGTCTGAGATTGGATGGCTGGTAAGATCGCGGTTCCCAGTTGTTTCCAAGGTCTCCTGCATTTGTGTTTAAAATGCTGAAGTTT
TCAAGGTGGTGTGTGTTGGGAGTCTTGGATAAGTGCTCTGAACATCGCTTGGGAGCTCCTCCCTGGGAAGTGGGCATTTCAAATTTCGAGCTTT
TTGTGGAGTGAAGATGGTGACTGGACGTTAGCTTTGCTCGAGGCTTGCTTTCTCACTACTTCTTTTTCTCCTTTTTCTTTTTTCCCCTTTTGGA
GACTGCAACTTTTCTCTCGACTTCTACTAAAGGATGCACTCAGCATCATGACTCCAGTCCACTATCTTTTACACGTTTTCCTAAATGTCAGCAT
AGGTCATTTGGCAAAGGGATGCTTTAGAAAAGAGCACTAAAAAGTTGATTGCATATGTTTACGCAGTCACTTGGGCTTGACAGTGATATGGCAT
GATCTTTTTCCCAGAGATTTACAGTTAATAATTGGAAGTAAACAAGTAGAGAGACTTTGATCATTACCACTTGCAATAAAAGGATTTAGTGGGC
TTAGTGGTCACGGTTCAATATATGAAAGAGAAGCATCTATTAACCCCTTCCCTTAATGTGGGCAAAATTCCCAGCATATTTATCCAAATGCAAG
TCTACGTTGGTGGTGTGTACAACGTTATGAGTAGTTCAACCTATATTTTATAGGAACCTTAACTCCCATGTTCTGGAAACACATTATAAGCTAC
ACTAAACAGTTGATATTTAACCATGCAAATTATTGACAGTATATAACAAGTTATGAGACTCTATATGTTAACATTTGAGTTTTAAGTAGACCCA
TCAGAAATCCCTGGGATTGATTTTATAAACAAAATTATAAGTTTTTAGATTTGACTTAATTCCATACATCTACCCCAAAGTCTACTCACAACTG
TTTGAAATAACCTACTTGTGTGAGTCTTATGCCTAGTAAAATATCTTTAAAGCCATATACAAGTCACTTTGTATGCATGTATACACATAAAGCT
TTTGTAACAAAATTAAGCATTTAATTGTACATGATAAATCCATTAATACTCTATCACTAACTTCTTGGATGTATGTTAAGTGTGCAGATAGGCT
TTCTCCCCAGGAGTCAAATTAACCCCAACTTTACCCATGCTTATGTTGGGCTGTCAACCCCAACTGGGGCTATGGAGTTCATTTGCATTTCTTA
GATTGCAGTAGGCAAGCTGATGGTTAACATAAGAACTTGCTTCTGTTTTCCTTCATTTTGACATTCCTGGTCTTACGCCTCCACCTAAAAACTG
ACAGCCCCAACTGAGTGTGTTACAATGGCAAGTTTTAACGGTTGAGTATTTTTACCATGATATGTTTCCTGCATTTTTGTTTGCTTAACTGGCA
GTTTCTGGGTTGGAACCTGGAAGGTTTTCACTTCTGAGGTGGAAGAGATTGTTTCTCAAATCTTATCAAGCACGTCAAACACTTTGGAAGTCTT
CATTAGGTTGCCACTACTTTCAAAGGGCATAAAAACATAACTGATTTTTATATAAGTAACAGTGGTATTTATTAGCATTATAAATGGTCACAGG
ATTGTGGCCAAATTCTTCTGAACTGAGGTACAGCAATTGTGTGAATTAAGACTTAAGATGAATTTGAAAGACTTTTGGTCAAGTACATGTGACT
TAAATGCTCAAAGCCAACCGGTAGATAACCCTCCACTGAGGTTTGATTGTTCTCCTTGTTAATACTTGTCTTTGTGTGGTGTTGGTTTTCTGCA
AGCTAGAGGAGAAATAAAGGCAGCTATTTTTATTGGAGCCCCTTCATGAACTTCGTGATCGGTGGTTTTTTAGTGCAGTATCCTTTCACAGGCA
AACAGAAATATCAGCTTGCTATCTTTCTAGGAAAGATCCTGTCGTTTCACAAGGAGAATTTTCTTTAAGCACCAAGAAGGCTGTTCTTTAATTT
ACCAAGTATAATTTGATTTAAATATTGCTACTCTTTAGGGTCATTGCTCTACCTACAACTCCTCCGTGCCCCGCTTAGATTTATGTTAATATGT
TGACAATATTCTCCAAAACAAGTAATCCAGAGATAACTGAGGAGTCAGTCACACAAGAAGTTTTCTGGTAGACGTCGCCAGTTGCCACTGTGGT
CACTTTCATAACAGAGGCTTTGTTTTGTTTTTTACCATTTTCTAGAGGTTACCTCTTAATTTCAGCCATTGAGCTGTGTTGATTCGTGTTTCCC
TTTACTGCATTAATACACCTTAGGAATTAAAACCGCAGGTATATCTTACTGCAAGTTTCAAAAGGTCCTGGATTTCCATTCAAGGAAGATTTGA
GAATGTACTTCAGTGAGGTCTGGAAGCTTAGTCTTATATCAGGTTCTGAAATGAATGAAGAAGGGGGGTTGAAGTTTATTTTTCAAGAATACTA
CACGAAGTATTTGACTTCCCTGACTGTCGGCTGGTGTATTGTTGCTGACACTTCCCCATTGTGACTTTCCTGTAATACAGGTATCTGTTGGGAA
TATCATTTCATCGTATGGGGGGTTTTACATTTGTTTTTCTCTCCTGTCGCTGCTTTGGGTTGACAAGCTGTTGATTTAGCGTTGGAAATGGGCT
GGTCACATGGTTCTGTACATATTGTCACTATTGTACCGATTAAAGTACTTTTTCTTGCCCCCAGCATAGTTTGTCCAAATAAAACACTGGGCTT
TGCCCTCTCCTCTTCTTGTAAAACAGATTTCAGTTTTCAGCCTTTCCAAAACTTTGTATCGCTCATTTAATCTACAGGAGAGCGTCACATTAGA
GAAGCAAGTTTTAAAGAGGTTCGGTTCAATAAACATCTCTGCAATTTGAGCCCTGACGGTTTCGGGCTAAGTGCACACTTGGCAATTCTCAAAA
AATCCCTTCGTGAAATTTCTGACCGAGTGATTGCAGACAGGACAGACCGAGAGAGTCCAGATCTCAAACCAGCTCTGCGCACCAAGTCGACGTG
AAAAGAGGCCCCCGGAGTGTCCGAAATGGCCGGGAGTGCGGTTTGCATTTTCCTTTCGCGGGCGCTGCCGCAGGCTCCGGGGTCCCCGCCGCCC
AGGACTGGGTCCCCGGTCCCCGATCCCCGCTCCCCGCTCCCCAGCGCTGTGGCCTCGTGGGGCTGCCCCGCCCCCCCGGCGTCGCCGCACCCAC
CCCGCCCCCGCGCCGGCCCCGCCCCCGCGGCGCGCCGCCCCTCCCCCGCCCGCCCGCCCGNNNNNNNNNNNNNNNNNNNNCCCCTCCCCCGCCC
GCCCGCCCGCCCGCCGCCCCGCACGCCCGCCCACGCTCGGGGCTCGGGCCGGGCTCCCCGCGGAGTTGCAGCGGTGGCCGGATGCCAAGTGTAA
GTGTAAGTTGCTATGGAAACCCCCACCGAGGCGAGTTCCGAATCCGGAGCGAGACGGAGCCCCCGGCGCCGCCGGATCCGCCCCTCGCATCCCG
GCCCCCGGGCGTCCGCCGCGCTCAGGCCCCGGCCCAGGCCGACTGGAGGTGCTCCTCCTGCGGCCCCAGCACCCGGCTCCGGAAATGCCAGGGA
TGCAGGGAGGGCAGAATTGTTTTCCCTGCGCGTCGCGTAAAAACGTGCCAGGTTCTGCTTGCTCGAACCGCCTAAAACAACCGGAACCCCCGAT
TTTCACTTGCAGATTCTTGAGGAAGTTTTATAATAAATTCGGAGACCTGGAACAAGCCCTAGTGGTTGGTAAATATCCGCGGGGATTGTGTGGC
GTCTGCAGCAGCCTTGGGGCTGCTGGGCTCGAGGACAAATGGAAGAAAGAGACCCTAATACGCTCACCTCCCAGCCCCCACCCCAGACTTTCTT
CTACCTACAGCAATGATCTGAGAGACCAGAAGTAATGTTAGTTGGATCCTTTTGCATCAGTTTGGTAGAACTGATTCCGATCTGATGAGTAAAT
TCCTTCTGTAGAAACGTTAGGACAGCCCAGCTGTACAGTGTTAAATGAGTTTTATAAATTCAGTGTTTCAGGGTTTGTGATTACTAGATGATCT
CCATTCTTGTTCTGTGAATTATGGCCATTATTTCTGTGTCTTGCAGGATTTTTTATCAAGCAGAAATGCATCGAACAACGACAATCAAGATCAC
TGAGCTAAATCCCCACCTGATGTGTGTGCTTTGTGGAGGGTACTTCATTGATGCCACAACCATAATAGAATGTCTACATTCCTGTAAGTACCGA
GCTTTAGCTCTCTTTTGTATCATGCGTATTTCACAGTTCGACCTGGAATTTGAAAACTGTTAATCATTCCTGCAATCTGTGGAAATGTTGGTAC
AAAGTGGTGAAGGCATTTTCTTCTCTTGCATCTAATGACTTTTTGTTAATATGTATTGATTGTATTACAAGCATCCAATATATGTGTTCTCAGG
ATATGAATTAGCTTATTTAGTTGGAAATGCCTTTCACCATCTTGTTTTCTACTAGTTCTGGGTTTCTAACACCAATGATTTATCCACTCATTTC
TAAACATAAAAAAACTTCACATGTTCTACTTCTAGTCTGTAAAACGTGTATTGTTCGTTACCTGGAGACCAGCAAGTATTCTCCTATTTGTGAT
GTCCAAGTTCACAAGACCAGACCACTACTGAATATAAGGTAGGAAACTGTTGAAATTCCTTGTTTGTAATTATTATTGGAGTTCTATAATTTAC
TGAAGGCAACCCTCTTTATTTCTTCACAGAAAATTTACTCTTCAATACATAACTAATATGTGTGTGCTTTGTGCAGGGTAGCCGTTTAATTTCT
TCCCATATTAATGGTGACCAAGTTTAGACAAAGTTTAGTAGTATTCAAGTGATTTTAAGATTTTTTTTTCCTAACTGTCCTTTAAAAACTGACT
TAGTTCACCTTTTTTGCATTGCTTTATGGAGTTCATGACAGGCAGGAATAAATATTTAAAAGGGCTTCCATTCCTCTTTATTCCCTATTCATAT
TAGCTAGTTTAGTAACTTGAAAGGCACACTTCTTTTGACAGAGCAAATCTGAAGAATTTATTAGTGAATATAAAATATTTTTCTACTGATTTAA
AAAGTGCTATATACAGACAACTTTTATTCTCATAGTTTTTATCTTATCAATAATAGTTATAGACAGCATCACAATCAAGTAAAATATTATAGCA
CAAAAGAAGACTATAGAAACTTATGAACATTTTTAGTTCTTGCCATCTGAATTCATAGTTTTCTATTTTAATTATTTTTCAGGTCAGATAAAAC
TCTCCAAGATATTGTATACAAATTAGTTCCAGGCCTTTTCAAAAGTGAGTAACTTGCTTAGAAAATGAATTTAAAGAGATTATTCATTAGTTCT
TTGTGTTATGCCAAATGTTTACATCTTTTTTCCCCATTCAGATGAAATGAAGAGAAGAAGGGATTTTTATGCAGCTCATCCTTCTGCTGATGGT
AAACCTTTTAGGGGAGGGAAGACATTTTATATGGGGGGAGAGCTTATCTAGGAATTAAAATGTATTAAAATTGACTTTACCACTTCCATCCTCT
TATATATAAAATTTATTAGGCATCTGATTTTTAAAAATTACATTTCACTATATCCTTATAGCTGCCAATGGCTCTAATGAAGATAGAGGAGAGG
TTGCAGATGAAGATAAGAGAATTATAACTGATGATGAGATAATAAGCTTATCCATTGAATTCTTTGACCAGAACAGGTAAAATCTTTAGGCAAT
TTATTTTATGCTAATGTTTTATTAGATTGTTTCACTAATAAATTTTCTCTTTATTAGATTGGATCGGAAAGTAAACAAAGACAAAGAGAAATCT
AAGGAGGAGGTATGTTTCATGTTACAAAAACATATAGAACAAACATTGTTTGGAATCCAGATTTTATCAGGGATTCTCTTGCCCTTTAGAATAT
TAGGCTGTTAAATAGAAGAAAATGGTCATGTTTAATGTTTAACAAAGGTCTACATGTGATATTTAATAATTAGAATTTATTTTATATATGATTG
TTAGTCTTACAAAATATTTTAAGTAGTTCACAGTTAATACTTTGGTATGCTTGAAGATTATATTAGGAATCTGTTCATTTAGTATTTGTAATCA
TAGTAAACTCAGAGTGTAATTAGTTGAGAGGTTGGTCACCTCCAATTTTGTTTGATACTAGTATCTTTTATATTCAAGCAATCAAATTGAAACT
TTCTTCATATCTTAAAGTAACTGAAAAAGTTACTTTTCTAAATGTACTTTTAGGTGAATGATAAAAGATACTTACGATGCCCAGCAGCAATGAC
TGTGATGCACTTAAGAAAGTTTCTCAGAAGTAAAATCGACATACCTAATACTTTCCAGGTATCTACTTTTATATTCTTCTTGCATTTTACATAG
ATTTTACAATTTTAATTACATTTTTCACTTTGGATTTTGAACCCAAAAAAGCAATAGAAAAAAAATGTGAAGTTAGTGCTAAAGTTGAAATTTA
TCTTAAAACATTTGGTATACATTCTCTTTTATGCTATGAAAACAAATCCCTTAACACTCTCTAGAAATAATTTTTTCTCATTTGAACTTTCAGG
AGTCTTTCTTTGTTAAAATGTTTGGAGAATATGTTTCTTAAAGCACTTTTGGATTATATTTAAATGACAAAAAATCTACATTTACCTTTCTTCT
TTTATTACTTAATAAAAATATTTTTCACTACATTGATGTCATGTATGAGGAGGAACCTTTAAAGGATTATTATACACTAATCGATATTGCCTAC
ATTTATACCTGGAGAAGGGTAAGTAGCATATCTCTTCTTAGATTATGATGGTAAACTATATTTAAAGAATTAAGAGTAATTTTTTTCCTTTTAA
CTTTGTAGAATGGTCCACTTCCATTGAAATACAGAGTTCGACCTACTTGThAAAGAATGAAGATCAGTCACCAGAGAGATGGACTGACAAATGC
TGGAGAACTGGAAAGTGACTCTGGGACTGACAAGGCCAACAGCCCAGCAGGAGGTATTCCCTCCACCTCTTCTTCTTTGCCTAGCCCCAGTACT
CCAGTCCACTCTCCTCATCCACAGTTTCCTCACATTTCCAGTACTATGAATGGAACCAGCAACACCCCCAGCGGTAACCACCAATCTTCTTTTG
CCAATAGACCTCGAAAATCATCAGTAAATGGGTCATCAGCAACTTCTTCTGGTTGATACCTGAGACTGTTAAGGAAAAAAATTTTAAACCCCTG
ATTTATATAGATATCTTCATGCCATTACAGCTTTCTAGATGCTAATACATGTGACTATCGTCCAATTTGCTTTCTTTTGTAGTGACATTAAATT
TGGCTATAAAAGATGGACTACATGTGATACTCCTATGGACGTTAATTGAAAAGAAAGATTGTTCTTATAAAGAATTGGTTTCTTGGAAAGCAGG
CAAGACTTTTTCTCTGTGTTAGGAAAGATGGGAAATGGTTTCTGTAACCATTGTTTGGATTTGGAAGTACTCTGCAGTGGACATAAGCATTGGG
CCATAGTTTGTTAATCTCAACTAACGCCTACATTACATTCTCCTTGATCGTTCTTGTTATTACGCTGTTTTGTGAACCTGTACAAAACAAGTGC
TTTTTATCTTGAAATTCAACCAACGGAAAGAATATGCATAGAATAATGCATTCTATGTAGCCATGTCACTGTGAATAACGATTTCTTGCATATT
TAGCCATTTTGATTCCTGTTTGATTTATACTTCTCTGTTGCTACGCAAAACCGATCAAAGAAAAGTCAACTTCACTTTTACAATCTCTATGCCT
AAAAGCGGGTACTACCGTTTATTTTACTGACTTCTTTAAATGATTCGCTTTTGTAAGAATCAGATGGCATTATGCTTGTTGTACAATGCCATAT
TGGTATATGACATAACAGGAAACAGTATTGTATGATATATTTATAAATGCTATAAAGAAATATTGTGTTTCATGCATTCAGAAATGATTGTTAA
AATTCTCCCAACTGGTTCGACCTTTGCAGATACCCATAACCTATGTTGAGCCTTGCTTACCAGCAAAGAATATTTTTAATGTGGATATCTAATT
CTAAAGTCTGTTCCATTAGAAGCAATTGGCACATCTTTCTATACTTTATATACTTTTCTCCAGTAATACATGTTTACTTTAAAAATTGTTGCAG
TGAAGAAAAACCTTTAACTGAGAAATATGGAAACCGTCTTAATTTTCCATTGGCTATGATGGAATTAATATTGTATTTTAAAAATGCATATTGA
TCACTATAATTCTAAAACAATTTTTTAAATAAACCAGCAGGTTGCTAAAACAACGCATTTTATCTAAAGTTATTTTAATAGGTGGTATAGCAGT
AATTTTAAATTTAAGAGTTGCTTTTACAGTTAACAATGGAATATGCCTTCTCTGCTATGTCTGAAAATAGAAGCTATTTATTATCAGCTTCTAC
AGGTATTTTTAAATAGAGCAAGCATGTTGAATTTAAAATATGAATAACCCCACCCAACAATTTTCAGTTTATTTTTTGCTTTGGTCGAACTTGG
TGTGTGTTCATCACCCATCAGTTATTTGTGAGGGTGTTTATTCTATATGAATATTGTTTCATGTTTGTATGGGAAAATTGTAGCTAAACATTTC
ATTGTCCCCAGTCTGCAAAAGAAGCACAATTCTATTGCTTTGTCTTGCTTATAGTCATTAAATCATTACTTTTACATATATTGCTGTTACTTCT
GCTTTCTTTAAAAATATAGTAAAGGATGTTTTATGAAGTCACAAGATACATATATTTTTATTTTGACCTAAATTTCTACAGTCCCATTGTAACT
GTTGTTTCTAATTATAGATGTAAAATGAAATTTCATTTGTAATTGGAAAAAATCCAATAAAAAGGATATTCATTTAGAAAATAGCTAAGATCTT
TAATAAAAATTTGATATGAAAAGCACAATGTGCAGAAGTTATCGAAAACCTATAGAGGATTACAACAGGTAAACCTTAAAGAGAATACATTGCT
GACTTATAGTCATGTGGCTAAGAAGTACATGCTTTGTTGTAAAATTGCTTGAAAGCCCATTCAAAGATGTATCTGTTTATTTACAGTCTTTGAA
GTAAAAGTTACCAATGTTTGCCAATTTTGGTGAATTTAACTCATTTTCTAGGCTTAAGTTTTTCATGATGTTTAAGACAAAGAAAAGTTAATCG
AAGTGTCAAGTGATATTTTTACCTTGGATGATCATTTGTATAAAGAATGAAAAAATATTTTTAAAAGGCTAAAAACTTTAAATGCTAGTGGACT
GTCATTTCACTTACATAAAACCAAGTTATCAACATGGAATTCAAATTTTACATCTCAATCTCTTTATGATGAATTTATTTTTGAAATTGCTTAT
TAATCAGATACTGCAGTTATGGCTTACTTAGCACTAACCATATGTGAAAATGTTTCCATTTATTTGAAAAATGATTTTTTTTCCTCCTCAATTA
GAAAATGGCATAAGCTTAAAACCATATTTCAGCCAAAATTTTTTCTGTTAATGAAAGATTATAATGTTTTGTATTCTTAAATAAGCAGCTATTA
CAGTTTGACAATTGAGATTAAAAACCTTTGCTAAATTACCATGTTACAAATTTTCAACCTTATGAAAACCCATATCAAAAGCTCCTAACTTTGT
AACTTTTAGTAGTCAAAGCAGGGTACAGGAAAAGTCAGCTTTAAGTTTGACTTTTGCTTCTGAAAATATTCTAGAACACAAAGTTAGTGCTAAC
AAAATATTTCAGAGAAAATGCCTAAATTGATTTTACAGATTTTTAGCTTAAATGCAAAGAGAAAAATGAGGCTGGTTGCTGTTGTGTAAATAGT
GATCTATCTTGCTTTACACATCCATACCATTTTACAAGTACTTTTTGCTTTTAAAATTGCCGGCCGTTAATTGCACTGCATTGAAAAAACAACT
GCCCTTCAGTTGTCTCAACCTTCCAACCATATTGCTATTTCCATATCCATACGTGATAATATTTGGACCTCTGGAGGGAAAATGCAACAGGCAG
AGTAAGCTGGTAGGAACTTTCCTTCATCCTTTCCTCTTTTCCGAATATTTTGAACGTGCAGTTAATTTGTCAAGTCTTGTTACACTCATTGTAT
GGGCCTATTAACGTTTTGTTATGAAAACAATAGGAACTTAAATACCCATGTGAAATGCGCCTTGCATTGACCAACACGAAAGGAGGACGTTTGG
TATTTTAAAATAGTTTATTTTATGGTATCTTTTGAGGGGATACGATTTACTTTTAGAAACAATCTACACATGGAATCCAGTCTCTTAAAATCCT
GGGGTGAAGCAATTGTTAATTTTTCCATTTCTCTTTGTATTTTAAAGGTTTCATAAATGATTTACATCTTTTTCTTCCTAATACACGAAGAATG
ATAGTTCGAAATTCTGATTTAGTTTAGTACTTAGTCAGAATGCAGGCCCCATAAGATTTTTAAAATACTTTCGCTGTTTTCCCCACACAGTCTT
CATTCCATTTTATCCAAAACAAAAACGTCTTGATCAAATTACATGAAAAACGACTAGACATCATTTTAGATCTGTGCTGCATCTAACAGTATAC
TAATTAAAACCTTTCAAAAACCGTCTATGCCTCTAAGACACTAATTTGGTGCAAAGATCACTGAACCCAGCTCCAACTTAAAGCTCTTGAGATA
CATGATTTCACTTTAGGATGTTTCTTCATGTACAAAATGAACACAAACACAGGTGATAATTTCTAAGGTTGCTCTAAAAAAATTGACTCCTCAA
AAAAAAGCCCTTGAACTAAATTAGGACCACAAACTTCAAAGCGAGGAGCCGAGTTCTTTACAAGGAATAGAAGGAGATATCCATATATTCCAGC
CTTGCAGAGAATCAGATTTGCAAATACCCACCGCGAAGACCTTGAGCCAGCCGATCTCCCTGCAAATCTCAAGATCGCCCAGTTTTGATTATGA
AAGTCTTCCAAAATGTTTACACAGTTGACTGTTTCCTGTTTCCCCAGTTAATTTTTTTTAACGTTTTTTTCTTCTTTTGGTTCTTGAAACGGAT
TTCCTTTATTTGCACCAGTTTCTTTTTCTTAGTGTGCAATTTCTAGAGGTCTCACATGAAGAACTTCACTATCAACTCTGTCTTCATCAAGTCG
ACGACATCAGAAGTCATGTTATATTTATCACTGGAAAGACTCCCCTGTAGCACTCTTGTGAGAAGAGTTCTGCAGAAGTTCAAGTGGAAAACTC
ATTCGGAAGTCGCTTTAGCCCTCCAAATAAATAGTAAGCGCTCAATAAAAACTTCCAACAAACCAGTTAGAACTACTCTCATTCTTCAGCCCCT
AGCCTTTAAAAATGTCCCTTCCGGGACTATCCGAAGTCACTTTCATAAATAGCAGGTGTTGTTCCCTACATTTCAGTAAAATGCTACTTCTTGC
TCTGATTGTCAGGTCATCATGGAATGCAAACTTCACCGTGGACGTGATTTCAACGTTGGTGCGGATTTCCAGATGTCCAACTCTGTCCTCAATT
AGCCAGAACTCAAGTTGAGATAAGAGACTTGGGAGGCTGCAGACCCAGGTCATAGGGAAGCGCGTCGGAAAGTGGGATGCCAGGAATCGGATGG
TGGCCGAGACCGACACCGAGGTTAGTGCAGGTGGAGAAGTAGGTCCAAAAGCCCAAGTCACAGGTCCGAAGTCCTCAGGAGCGGAAGATCTTCC
AACTAAGCCGGAGAGCGGGGCGCACCGCACGGCGTGGAGCATAACGCCCGGGCGAGGGTGATTGGGGTGGGAAACTCCACTGCTTTCCAAAAAA
CAGGTTTCTTAGGTTCTAGTGCCCGGTGGAAAGGGCACAGTACACCGGCCTGGGGGAAATCACCTGACTTCTCCTTTGGTAAAGGCCCCCTTAC
CAACGCTGCCTCTCTGGTAACGGGGTCCTTTACCAAAACACACCTTAAAGAAGAAACAGCCTCTCTCCTTTTCCTTATTTTCTAATTAGCATCT
TACAGAGGAGTGGAAACAGCTACAGCCCAGTCCCCTGCTCAAAACTGCGCCACCCCAGTTCGGCCCTGCTGGGCGCGCGAGCCAAGGCCGCGGG
GCACCGGGAGGCCATTTTGCGCGTGCGCTGCTCGCCTCGCGCCGCCCTCGGCTCTGCGGACTCGGATCCCGCCAAATTTGAACGCGAGATTGTC
AGGCCCTGAGGGGCTTGACGGGCGGGGGAACGACGCCGCTCTCCAAAGTTGGACCCCCTCGCGAGCGGCGGCGACAGCCGGGTGCTCGCTGCCT
CCCCAGGTGCTCCCTTTTCCCGCCGAAGCCCTCCACAGCGGCAGCCCGAGGCGCAGCGACGTGTCCCTGTACCCCGAGTTCAGCGCGGGCGGGA
AAACGACCTGCACCCGGGGAGGCAGCGGCTTCGCGGGCAGAGCCCACGGGAGCGCGCCCTGCTAGGAGCCAGGCCGGATAATCGCCTTTCTTTG
TCCTCCTCCCTCTTCGAGTCCAATCAATGCCCTTTCTCCTTAATGAACCAGCTCTCCTTGGAGTTTGAGGTTTTGTTGGATGATTTTAAATAAA
ATTATTAAGTTATAAAGTGGCCACCCTGAAGGTTCCCGAAGGCGACTTCATGTCTGTCACTGGAAAGGCCTAGAGAAGAGGGTCCTCCCGCTGG
GCTCCTTTAATAGAACGCGCTCGAATCCCCTGGGAAAGAGCCTTCACTGGGTGACAGGGCTGACCAGGGGTGGCTGCGCCCCGGGAATCTCAAG
ATCTGGCCAAAGGCTCGCGTCTCGGGACGCGAAGTCGACGCCAAAATGGGTCCCCGGACAAGGCGACCCTGGGAGTGCCGGCGCCCCCGGCCGG
GCAGAGGAGCGGGTGGGCCGAGGCTGGGACATCGCCTCCGAAAGCTGCCGGGACGCGGCGGCTTCCTGCAGAGCCTGCGCCTGCCGGATCCCCA
CAACACAGAAGCTTCTCGGACATGGGAGCTCCCCGTGCGCCCTAAAACCAGGAGAGGAAGCGACGACTTGGGAAAAGGGACTGGGGAAACAGCG
GAGAAGTGAAAGCGGCCTAAAATGGGCGACGGCGGGCGAGTCCTCTTTATCAGTGCACCAGGCTGCCGGACCCGCCATTTGGTGGCGGATCTCG
GTAGTTCAGTAGCACGTTGTGCTGAACGTCACAACTGGCTTGTCTACGTGGCATCGTCATTTCTTAACCGCGGTTTTACGAAATGCAAATTTCC
CCCTGGCCTTCCTCCTCCGCGGCCGTCGACCCCCCTCGCCGCTCCGGGTGGACCCCTCCGGGGCGCGCCTCCTCCCTCGGGTGGTGCACCCCCC
GCGGCCCGAGACCCGGGGAGGGCCGGGGGTACTTTCTGCGAGGCGCCTTCCCCGCGGCTTCTGCCCGCGCCAAAGCCTGGTGGAATCCAGCGCA
GACCTAAAGCACGCTTCACACCCCGATTTTTCGAGACTAGGACGACTCTCTGAGCCAGCAGCTTTCCTCTCCCTCTCGGGGAGAATCTCATTTC
CTTGGGGTGGTGAGGGTGACGGGCACTGTCTTTTGGCCCCGCGTGTCCGTTCCCCGGTCTCCCGCCTCACCCCTCTGCGAGGTGAGGACCGGAA
ACGGCGAGCTTAGGCCTGGCGGGAAGGAGCCTACCCGACGAGAGGGCTCCGCGGGGAGGGTCGGTTGCAATCCCGCCCTAGCGCCTCCTGCTCT
GCCCGGTCCCCACCGGGGACGGGGAATGCCAGTCATTTCTGTTGAGTGCTAGCAGGGCCGGTGTCACCACCTCGGGTGGCCGAGGCTTCGAGGT
TTTCATGAAAAGCCCCCGAAGCGTGAGGCGCCCGCCCAGTGGAGAACAAAGGGCCGACGGCCGAAGGCGAGGCGAGGCAGCGCGCGCGGCTCCC
TTGGCTCGACCTAGCTGGGAGTCGGGGGCGCGGGCAGGGCTCACTCCCGGCCTACAAACTGGAGCCCGCCACCCCCGCCCCGCAGGCGACCGCA
GCGATCCCATTCTTGGAGCCCGAGCTGCCATGTTGCCTTCGCGGAGGCCGCCAGTCACTTGACGCTTCCGACACAGCGAAGCCCCCAACCTGAG
AGCCCTTCGGCCGTCTTTGCCGCACAGCTGCAGTCAAGGCCCGGAGGGACTGCGGGACGCGGGCGGGAGCGACAGCCCTGTGGGCTGCCAAGCC
GGCGCGGCCGCGCCGCGGCAGCCGCTTCCCTTCCCACCTTCGTTCCACGGGCTCCGGGGCTGCGCGCTCGGCAGAGGCTCGGTTGCCAGTAGCA
ACCACACGACGGCGATTTGCAGCCAGGGCCGCCGCCGCAGCCGCTGGTACCTCTGCCTCCTCCTACACCTCGGGCTCGAGCATTTGAAACCCTG
GGGGTTGCCTTCGGTGACATCTCCCGCCCCCACCTCCAGTCCTCAGTCTCCAAAATCCTCAGCTCTGCTCAAAAGCCAGCGCCCCCGGCTGGGC
CCTGCCCCCACCGCAGACAATAGGAGCGGCTGGGACCGCACAGGGCCGCGCGCGCCCCCAGCAGCCCGCACCTCAGCCCCCAAATCCGGGCGCG
TCGCTGAGTCTCAGCCCCAGCTGCCCTCTTCGCGGAGTTGATCACCAATTCTTTTTCAGTGGCCAATTAGAAAGCTTTTCTTGACATCAGAAAT
AAGGGTGGTGCCAAAGAAAGAAGTCAAACGCCCACTCCCTTTCTAATAACATCGACACAAAGAAGAGCTCATCGTTCCGTCTGCGCTTGCTAAC
AGTGGCTTTCCTCTGGGTTAGAGCCACAGGCACAAGCACCCCCTGCTATCTTTCTTCTTCGCCTACCATGAATATTTTGCATGAAATCCAGAAT
GTAACCATTTGCGTTGACTTAACTGTTGCATCTGGATACTAGTTATTTATCAGTGGATGGAACACTTTTTTTTCCATTTCATTGGATGATCCGC
ATCAACCCTTGGCAGTGTCCACTGCGTGCTTTGAAACATAAAGAAAACGACTGGTATTAACCCTTGAGGATCTACTTTTTCTGTGGCTCTAGAA
CACCAATTTAACTTTTTTTAACAAGAGAACTCTTGTTAATGTTTTAAGCAGGTTTTATTGAAGAAAAGTCCTTAGAGTATCTAGCTAAGTCTTT
TATTTAGCTACTAGGATAACTCGGGAAATATCTGCATTCCCAAATTTCTTCGCTCTTAATAATTGTCCCGGCTTTGTTAGCTGAGTGGTCTTAA
TGGGTTAAAATTTCTTGTAAATCACAAATGTGCTGAAATTTCCAAGTTTAAAGCTCCCTGAGGATAAGCACCTGCCTTATTCATCTTTGTAAGC
GTCAGAGTCCTTTGAACATAACAAATATTTGTCGGACTTCCAGGGAATTTCAGAAGTCTCAACAACTCTTATTAGGAAAAAAACTAACTTACTG
ATAACTAGCAGAAAGCTTCGATATCTTTAATCAACTCAATCAAAAGCTAAACAATTTCCAAGCTGGTAGCATTATCAGATATTCTCATTCATGA
ATATTTAACTTAATTATATTTCAAATGCCATAGATTGTATGTTAAAATATCTGACACATGTTTTCAGGATTCTAAGGAGAATGACAAATTGTAC
ATAAGAACTTGAAACTGCTAATTTCTCATCTAGTGTCTAAAAATTTGGTTAAATACCCACAACTAAGGTGCAACTTTAAAGTGCTTTCATACTT
TCTGATAGCACAGACATGTCATTAACTGTCTTTACAGCTGGACAGGAATGCTTAAGGATCTACCGTTGCATATTGCAAATCTACTAAGACAAGG
CACCTTGAAATATCCTCTTTAATTTAAATAAGCTTTAAAGCATACTGAAAATAGTATGTCTCAATCACAAGTCATGGTAAACTCAAATTAATGC
CTCCTGAAATGACTCTTACGTATATTATTAAAAGAGGTTCACCTTTAAGCTTCACAAAATATCAAGTTCATTAAATAAGACAGTTTACCTAAAA
GAAGAAAGCACTGCTTGGTTCCTAGAGGGAGCATAACGACCTGTAGCTTGCCAAACCACCCCCATCAGCACCTTAAAAATAGGAAAGCAAGGCT
TTAGTGAAAAGTTCCTTGCTACCGTCTTTCAGGAAAGAAAAGCTCCAACAACTTGCTTTGGACCAAGTTAAACAGATTATCTCCCTTTTTCATT
ACGACTGTCTTTCAATGAATTGAAGGATAAGATCAAAGTGCATTCATATTCATGTGAATTATCGCTATTGACTTGAATAATGATGGATTTCATT
GTTTTATTTAAACTGAAACCAGGAACGCTGCTGCTTTTGACGCTACACCCCTAAGGCTGTAGAATTTCTTCTCTGAGAACTCAAAAGCCAAGCA
ATTAGGTCTGACAACCACGTCATTTCCATAATGTACTTCCGTACAGAGGACTTTTTATTGGAATCGGTTACATTTCTATAGAATCTTATTACCA
CTCAATTTTTGACTTCTTTCCAACCTTCTCAACAAAACTCCTAAGTTCGGCAAGTACACTTCCAAAAAACTTATGAGTAAATTGTACTGAGAAG
ATACAGTGATGTTTCAGAAGCTTTTATTAATAGCTGTTCATGAATCAGACAAATTAATGTTATCAGTAATTACCTCAGGAGAGTGTTTTTGTTT
TGAACATGATACATTACAGTTTTTCTAGGGTAACTGAAGAACTCAGTTACAGTAACCTTGGCAAAATGTGTTTACCAGCTGTTTCTTTCTAAAT
ATGCCTCATGACAATTTTCTTAAGACATTTTCATTGGGGTTTTCCTCAAACTGTCCCATCATTTTCTAAGGAGATGGAAGTTACTAGATTCAGT
GGAACTCCCCCTCACTTTCACCAAGTGATCACTCTGCTCAAATAATTCTTATTACAATGCTTATCAGTTTGGAGAACACTTTTTACGAAATTCA
TTACAATAAAGTAAGCTTGTCATTGAATTCTGCAAGCAACCTTTAAAATAGACTTAGGAAACTGCATTTTCCTGAACTTTAATATACACAATTT
ATATAGCCTCTCTAAACCTAAAAAGAGACTCTAGTGCAACAAAAGTCTATTACATTATCATGGTCTGCAGTGGGTTCTGGGAATATCCTTTCCC
AGAAAGAGAACTGATTCAGTTACTTGGTTTCTCTCTTAAACTTCTGCCATACATTTAAGGCATTACACATAAACCACATTTTAAAAAAAATTCA
ATGTTTTTCTATTCAGTTGGGCTACAAGAGATCTCTATTTTCATTTACCCATTGTCTGTTTTTAGTATTGCAAGACACTCTTTAAGCCAGGAAT
TATTAGTAAATTGTTTCTCTTTGGCATATGACCATATATATATTGAAATTATTTGCATACGTTCAGCTTTACTTCTAAATTTCTAAGACTGTTA
CCCTTGACTTATAAGTAAAATGATAAGCAATTGCATATTATTTCACTAGGGCAAAAAAAAAAAGCATTCCTTGAATAAAAAGCATTTCTTTATA
ATTCAATTCCTTAAGACCAGATTTGTCTTACTGATGGGAAAAGTATTACTATTTTCTTCATATAGGCAGTTCATCTTTTATATCGTTCATATAG
GAACTTTTATTTTTTCACCGTAAAAAAATTAGGTAATATTGGAGTAGGAGGTAAAGTAATCCAGCTTTAGCAATATTTAGATGTTTTCTCTGAA
ATGTCAAAAAGCAAGGGTCCAAACTGTAGTGCAAGCATCTCCTCTGAAACAATGAGATGTAGTGCCACCGTGTCCTCGAATTTAGCCTACGTTT
TAGCGGAGCTCACTCTCGGCTCAGATAATCTCTGGGGAAAAAACGGACAGATAGAGTGGTTAAACAGGAAACACTATTTTTTTTTTTCAAACGT
TTGAGAAAACTTGAAAACCATCTCTCGGTACTTTTGCAGTAATTTCCAGGAGTCAGAACATACAGTCCATTATTCTTCAGAGTAACCAACGCAC
AAGACATTTTGGCTTGGGACTTGATATCGCGAAATGACGTCAAAAAATTAAAAAAAAAAACGAAGCTCGCACATGCGCAATGACGTACAGCTGC
TTTTTATTTTTTTGTCTGTATCAGCTATTGGCTCCTATCCCCGCCTCTGGGATCCGGCCTGCGCCGCGGGGTCCTAGGGGGCCAGCAGCTGTAA
AGCGGTACGGGATTCCGCACCCTACTCCAGCAAGAAAGAGCCTGAAGCCCCTCCCCACCTTTCCCGGCCAGGGCGCTCTTCTACCTCCCGCGCG
CGGACAGCCGGGGCGAAGTACGCCGACCGCCCCCCCCGCAGCGAGTTTTCTGGCAGCGCTAGCGCCGCGGGGCCTCGGTTCCCGGGTTCCGGTC
TCCGCCGGCTCCGGGCTCGCCCCCGCGAGTTGGCCGCACCGTTCCCCCGCCCGCGTGGCAGCCGCTCCTCCGGGAGGCTCCGGCAGGGACCTTC
CCCCCGCCCCCCGACCGCCAGTGCCGCTCCAGCTGGAGGCCTGGCCCGGGAAGCAAAGTGAAAGGACACAGCCCTCCTTCCTCGCCAGCCCCCC
GCCCCCCCTTTCCCAGCTCAGGCCGGCGGCCCGCGGCGCGGAGGGAGCGAAAGAGTCGGGGCCTGCCCCCTCCACCGCCCGCATCTCGGCCGCC
GCACCCGGGTCCGCCCCGGGAGGCCCCGCGGGAGCGAACCCCCCGCCCGCTGGGCGCTTCCGCACTGACGCCTTGGGGCCCCCCGCCCCCGCCC
TTTCCTCCCGCTACACCCGCTGGGCCCCCGCCCGCGCTCCCGGCCTCCTCCCAGCGCCGCCTCCCCCGTTAGAAACTTCGCAGACACCGCGAAG
CTGCTCTTTGGAGTTGGAGAAACTTAGGAAGAATGGGAAAAGCCGAGGAAGTCGGGGAGGACCACGTTCTTCCCTTGCCCTCGGCCCGACACTC
CTGTCCACATTGGACGCGGCAGCCTGCCAGCGCCCAGGAGAGCCGACCCCGTCGACA
HUMAN SEQUENCE—mRNA
GAGAGGCAGAGATCGGGGCGAGACAATGGGGATGTGGGCGCGGGAGCCCCGTTCCGGCTTAGCAGCACCTCCCAGCCCCGCACAATAAAACCGA
TCGCGCCCCCTCCGCGCGCGCCCTCCCCCGAGTGCGGAGCGGGAGGAGGCGGCGGCGGCCGAGGAGGAGGAGGAGGAGGCCCCGGAGGAGGAGG
CGTTGGAGGTCGAGGCGGAGGCGGAGGAGGAGGAGGCCGAGGCGCCCGACGACGCCGACGCGCCCCAGCAGGAGGAGGCCGGCCGGAGGCGGCA
TGAGACGAGCGTGGCGGCCGCGGCTGCTCGGGGCCGCGCTGGTTGCCCATTGACAGCGGCGTCTGCAGCTCGCTTCAAGATGGCCGCTTGGCTC
GCATTCATTTTCTGCTGAACGACTTTTAACTTTCATTGTCTTTTCCGCCCGCTTCGATCGCCTCGCGCCGGCTGCTCTTTCCCGGATTTTTTAT
CAAGCAGAAATGCATCGAACAACGACAATCAAGATCACTGAGCTAAATCCCCACCTGATGTGTCTGCTTTGTGGAGGGTACTTCATTCATGCCA
CAACCATAATAGAATGTCTACATTCCTTCTGTAAAACTTGTATTGTTCGTTACCTGGAGACCAGCAAGTATTGTCCTATTTGTGATGTCCAAGT
TCACAAGACCAGACCACTACTGAATATAAGGTCAGATAAAACTCTCCAAGATATTGTATACAAATTAGTTCCACGCCTTTTCAAAAATGAAATG
AAGAGAAGAAGGGATTTTTATGCAGCTCATCCTTCTGCTGATGCTGCCAATGGCTCTAATGAAGATAGAGGACAGGTTCCAGATGAAGATAAGA
GAATTATAACTGATGATGAGATAATAAGCTTATCCATTGAATTCTTTGACCAGAACAGATTGGATCGGAAACTAAACAAACACAAAGAGAAATC
TAAGGAGGAGGTCAATGATAAAAGATACTTACGATGCCCAGCACCAATGACTGTGATGCACTTAAGAAAGTTTCTCAGAAGTAAAATGGACATA
CCTAATACTTTCCAGATTGATGTCATGTATGAGGAGGAACCTTTAAAGGATTATTATACACTAATGGATATTGCCTACATTTATACCTGGAGAA
GGAATGGTCCACTTCCATTGAAATACAGAGTTCGACCTACTTGTAAAAGAATGAAGATCAGTCACCAGAGAGATGGACTGACAAATGCTGGAGA
ACTGGAAACTCACTCTGCGACTGACAACGCCAACACCCCAGCAGCACGAGTTCCCTCCACCTCTTCTTCTTTCCCTAGCCCCAGTACTCCAGTG
CAGTCTCCTCATCCACAGTTTCCTCACATTTCCAGTACTATGAATGGAACCAGCAACAGCCCCAGCGGTAACCACCAATCTTCTTTTGCCAATA
GACCTCGAAAATCATCAGTAAATGGGTCATCAGCAACTTCTTCTGGTTCATACCTGACACTTTTAAGGAAAAAAATTTTAAACCCCTGATTTAT
ATAGATATCTTCAGCCATTACGACTTTCTAGAGCTAATACATGTGACTATCGTCCAATTTGCTTTCTTTTGTAGTGACATTAAATTTGGCTATA
AAAGATGGACTACATGTGATACTCCTGTCCGTCTTGGTTCAAAAGAAAGATTGTTGTTATAAAGAATTGGTTTCTTGGAAAGCAGGCAAGACTT
TTTCTCTGTGTTAGGAAAGATGGGAAATGGTTTCTGTAACCATTGTTTGGATTTGGAAGTACTCTGCAGTGGACATAAGCATTGCGCCATAGTT
TGTTAATCTCAACTAACGCCTACATTACATTCTCCTTGATCGTTCTTGTTATTACGCTGTTTTGTGAACCTCTAGAAAAACAACTCCTTTTTAT
CTTGAAATTCAACCAACGGAAAGAATATGCATAGAATAATGCATTCTATGATGCCATGTCACTGTGAATAACGATTTCTTGCAGCTATTTAGCC
ATTTTGATTGCTGTTTGATTTATACTTCTCTGTTGCTACGCAAAACCGATCAAAGAAAAGTGAACTTCAGTTTTACAATCTGTATGCCTAAAAG
CGGGTACTACCGTTTATTTTACTGACTTGTTTAAATGATTCGCTTTTGTAAGAATCAGATGGCATTATGCTTGTTGTACAATGCCATATTGGTA
TATGACATAACAGGAAACAGTATTGTATGATATATCTAThAATGCTATAAAGAAATATTGTGTTTCATGCATTCAGAAATGATTGTTAAAATTC
TCCCAACTGGTTCGACCTTTGCAGATACCCATAACCTATGTTGAGCCTTGCTTACCAGCAAAGAATATTTTTAATGTGGATATCTAATTCTAAA
TTCTGTTCCATTAGAAGCAATTGGCACATCTTTCTATACTTTATATACTTTTCTCCAGTAATACATGTTTACTTTAAAAATTGTTGCAGTGAAG
AAAAACCTTTAACTGAGAAATATGGAAACCGTCTTAATTTTCCATTGGCTATGATGGAATTAATATTCTATTTTAAAAATGCATATTGATCACT
ATAATTCTAAAACAATTTTTTAAATAAACCAGCAGGTTGCTAAAAGAAGGCATTTTATCTAAAGTTATTTTAATAGGTGGTATAGCAGTAATTT
TAAATTTAAGAGTTGCTTTTACAGTTAACAATGGAATATGCCTTCTCTGCTATGTCTGAAAATAGAAGCTATTTATTATGAGCTTCTACAGGTA
TTTTTAAATAGAGCAAGCATGTTGAATTTAAAATATGAATAACCCCACCCAACAATTTTCAGTTTATTTTTTGCTTTGGTCGAACTTGGTGTGT
GTTCATCAGTTATTTGTGACGGTGTTTATTCTATATGAATATTGTTTCATGTTTGTAGGGAAATTGTAGCTAAACATTTCATTGTCCCCAGTCT
GCAAAAGAAGCACAATTCTATTGCTTTGTCTTGCTTATAGTCATTAAATCATTACTTTTACATATATTGCTGTTACTTCTCCTTTCTTTAAAAA
TATAGTAAAGGATGTTTTATGAAGTCACAAGATACATATATTTTTATTTTGACCTAAATTTGTACAGTCCCATTGTAAGTGTTGTTTCTAATTA
TAGATGTAAAATGAAATTTCATTTGTAATTGGAAAAAATCCAATAAAAAGGATATTCATTTAGAAAATAGCTAAGATCTTTAATAAAAATTTGA
TATGAAA
HUMAN SEQUENCE—CODING
ATGCATCGAACAACGAGAATCAAGATCACTGAGCTAAATCCCCACCTGATGTGTGTGCTTTGTGGAGGGTACTTCATTGATGCCACAACCATAA
TAGAATGTCTACATTCCTTCTGTAAAACTTGTATTGTTCGTTACCTGGAGACCAGCAACTATTGTCCTATTTGTGATGTCCAAGTTCACAAGAC
CAGACCACTACTCAATATAAGGTCAGATAAAACTCTCCAAGATATTGTATACAAATTAGTTCCAGGGCTTTTCAAAAATGAAATGAAGAGAAGA
AGGGATTTTTATGCAGCTCATCCTTCTGCTGATGCTGCCAATGGCTCTAATGAAGATAGAGGAGAGCTTCCAGATGAAGATAAGAGAATTATAA
CTGATGATGAGATAATAAGCTTATCCATTGAATTCTTTGACCAGAACAGATTGGATCGGAAAGTAAACAAAGACAAAGAGAAATCTAAGGAGGA
GGTGAATGATAAAAGATACTTACGATGCCCAGCAGCAATCACTGTGATGCACTTAAGAAAGTTTCTCAGAAGTAAAATGGACATACCTAATACT
TTCCAGATTGATGTCATGTATGAGGAGGAACCTTTAAAGGATTATTATACACTAATGGATATTGCCTACATTTATACCTGGAGAAGGAATGGTC
CACTTCCATTGAAATACAGAGTTCGACCTACTTGTAAAAGAATGAAGATCAGTCACCAGAGAGATGGACTCACAAATGCTGGAGAACTGGAAAG
TGACTCTGGGAGTGACAACCCCAACAGCCCAGCAGGAGGACTTCCCTCCACCTCTTCTTCTTTGCCTAGCCCCAGTACTCCAGTGCAGTCTCCT
CATCCACAGTTTCCTCACATTTCCAGTACTATGAATGGAACCAGCAACAGCCCCAGCGGTAACCACCAATCTTCTTTTGCCAATAGACCTCGAA
AATCATCAGTAAATGGGTCATCAGCAACTTCTTCTGGTTGA

TABLE 18

MOUSE NOMENCLATURE
ICSGNM Runx1
Celera mCG13559
HUMAN NOMENCLATURE
HGNC RUNX1
Calera hCG401175
MOUSE SEQUENCE—GENOMIC
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNCTCTCTCTC
TCTTATACACCGACTTCTGTCTTCTTCAGTCTCGATCAACAGCTGGCTTCATTCCAGCCCCAGGCAGAAAAACTTACCATTTTTAACCGTCTCC
TTTGGCTACATTGTAGCAACTAGGATCAACTCTGCTTGCTGACTGGAGTCCAACTGAATAGCAACCTTAACTACTGTAAAAAGCCCTCCCTCTG
TTAACTGTTCTACAACCAGCTCATATGGGTTCCTCTCCCTCACTGTTCTGTTTAGAATGGACAATCTTAAGAGTCTATTTTAGGATTCTTCCTC
ATATTCTTGCTTCTGCTTCCTAAGTTGCTGGCAGAGTAATTGAATCTCTCCTGGTTGAGGATAAATGGGGGTGTGGAAAGGGGCCAAGGATTGC
ATACTGAGGAATGTTCTTCTACATACAAACCTGAAGCCAGGGGAAGGTGGCCTCTTACCAACCTCACCCTCCATCATTCCAATGCTGAGACACC
AAGTATAGTTCAAAGGTCATCTGGGAGACATGGAGACCATGATGAAGTATTAGGATAATCCATGAGAGGGAATCTAACCTGAGCAAGAAAGCTG
ACACATCCCCTCCGCAATCAGAACAGGGAGGGTGCTCCATAGTCCTGCAGCACAGAGCTGAAGAGCTGGCCCTGGGTTCTGGGTATGTAGAGAT
GCAAGGTTAGCAGAAGCCTCCTAGAAGGACTCGTGTAGAAAAGCTGTGGGAATCCCTTGTCAGGGTATATTCGTAAGAAATCTGACAAGAGGTC
AGAGAATTAGCCCTGGAGTGGGAGCAATTGGGATGGGCATGGGAAGAGGAGAGAAATCATAACAACCAATGCTAAAAAAAAACTACTCAGAGGA
CTTAGCAACAAAAGGAAGGGAGACACAAGTAAGTCCATGGGTTCGAATTCAGAGACTTGGTGTAACTACAATGAAAGCGGCAGCGGCAAAGCCA
GCTTGACATGCAGGACACAATTCTCAAGTGTTCTCTCTCTCTCTCTCTCTCTCTCTCTCTCTCTCTCTCTCTCTCTCTCTCTCTCTCTCTCTCT
CTCTCCCCTCCAGTCACCTATACACTTTTGGTGGACTAAATTTCTAAATTACCCACTCGGCTAGGCTGTATCCTTGGATGTAGTGGAACCCATT
GTCTTCATATTGAGGTTCAGTCCTCTCAGCTTATGTGAGATACCACACCCCACCCTCCAACTCAGTCTCTACTGGCAATGGCACAGTGACCTGA
ATTACAAGTGCCTAGCTTTGCTCTCTACGATCCTCCTCCCTGGAGCACCTTTGTCCAGCTCACCTCAGCCTCTCTAGCCCCTTCTCTGGGATTC
AGAAGGTAGAAGCTCTATTCATCTTCTAAGTCAAGGACACATCACCTGTGTTAGCACCAAAACCTCTTCTAAGCATCCCCACGTGCTGCTGCCC
CGTCCTCCCTTGTCCCTTCCCTCTTGTTCTCTGGACCATGCACACATGCTTGCCAGTAGCCATGATACCCCATTTGCTGTCTGTTATTTGTCCT
CTGTCACCAATAAGCCTAGAACCCTGACACATAAGGACCATTCTAGGAAGGCTGGGAGAAAGAAAATCAAATATTTCATGCTCTCAGAGAATCT
ACGACTTACCATTTGGAAAGACTAAAGGTCAGAAAAGACTAAAAGCCTATTATAGATTTTAAAATCTCTCAACCAAATAGCGATTGCCTGTCCA
AACACTCTCGGTGACAAGAAATAACTACAGGATAGAAATTCTACATATCTGAGATTCCCACATGAAGAATTTTTGAGATTTTTTTTTTGACAAA
ATATCTCTGCATAGCCCTGGCTGTCCTGGAACTCACTCTGTAGACCAGGCTGGCCTCGAACTCAGAAATCTGCCTGCCTCTGCCTCCCAAGTGC
TGGAATTACAAGAAATGAGGAGGAAAGAGGCGAAAGAGGACATACTAACAGTTAAATTTTTCTCAAAGCTGTCAATTGTACCTAAGAAGAAACA
TAGTTGGTCCATGCCTGTGGCTGTGCATGGGCTTGTGTATGTGTGTGAGTGTGTATACCAGTGCCTGTGTGTGCACGTGTGCATGTGTACATGT
NNNNNNNNTGTGTGTATGTGTGTGTACTCACATCTGCATGGGGTATGTGTGGTGTGGGGGTGAATGTCCATGTCTGTGCATACTTGTGCCTGTG
TCTGCACACTAAATCTGCATGTGTACATGTGCGTTCATGTGTCTCCACAGGCAAGTTTCCTAAGCTCCCTAAAGGGGAGGCTCCTGGCCTATAA
AATGGGAGTGACTATAGGATTCCCAGGATTAAGCAGGGAGACAATTCAATCACTGCTGGTTCTATGATGGCATACAGTGGGCAAGGAAGGGAGA
TCATGTCCTATCTAGCTTCCATTAATGAGTATGTAGCCTCTCGTCTCTCTCCAAGCCCCAGGGTCGCATCTTCCCCAGCTCCTGGAGGTGGAAA
TCCCTTCATAATTCCACAGGTTTGCCTTTATCTATAAGTGTCTGTTCACTGAGTTCAGCCAGTTTCCCTGTGTCCTCAAGCTGATGTCACCCAA
GCGCAGAAAGTTCTTCACAGTAACACACAGTCCTCTCCAAGCACAGTCGCTTTTGCCACGTTGGCTTTTCTCTAAGAATTGTCGAAGTATTAAG
TGTATACATACAGATCTGCGCTCCATTCTGCTTTTCCAACCACTCAGCCTGAGATGGAGAGGTGTCTCTGGGTCTCAAGAGAGAATGTCCAGGG
GGTTGGCTCTACCAGGTCTCTGGAATAAATTTACCTGTCCTAGTCAGTCCTACTGGGTCAGTCTGTTAGGTCCAATGCTCACCAGTTAGAACCA
AGCTTGCCTTAACTTCTGAGTCCATTTGTTATTTCTTACCTCATCTTACCTTAATAATTAGTGCAAATCGTTCTGTCCCTACTGTGTTCAAAAA
AAGGAAACTTTATGGAGCTTTTTTATCCCCTTGTAAACCAAGATCTTTTAAAAATTATTTTAAGGTTGTTCCTTGGAGQCCACACATTTAAGTC
CCTCAGTCTGTCATTAGAAGGCAACCCTGTGGAAGCCTTTCCTATTTTATTTGCTCTTTTTTGCTTTTGCAAAAAATATATGACTGTGGAGCTG
CAAACCTTGACCTCCTGCCTATTTAAACAGCGTCTGGAAATCTGAAGTAATTAGCTCTCTTCTTCTTCCTCTTCTTCTTCTTCCTCTTCCTCTT
CCTCTTCCTCTTCCTCTTCCTCTTCCTCTTCCTCTTCTTCTTCTTCTTCTTCTTCTTCTTCTTCTTCTTCTTCTTCTTCTTCTTCTTCTTCTTC
TTCTTCTTCTTCTTCACAACATCGTTGATAAGCCGTCTTGTCCTGCTATGGAATGTGACTACCCAAATCCTAAGGCATTGAGACCGACCATGTG
CCCTTACTGTCACTGGGTGCTGACCACTGTCAGTGTACAAAGCTAAGGAAGGGTAATTTCTAACTCACATTATCTGTAAATACTCAAGGGAATG
AGCACAGTTTTGGTGTGGGCTTCCCTGTACCTTAGAAGCAGCAGACTGCAGGGAGGAAAGTGTCTTCCTGGGATCTTAGCATATGCTGATGATT
TCCAACAATATTTAAGGCTGTGACATTTCAAGAACGAGTGGTGTCAATGTAAACAAACTGCTTTCCTAGCTGAAAAGAGGAAGGGAGAGGGAGG
GGAGGGAGGAGGGAACCAACCAAGGGGGAGGTGTTTCTGAAATGATCTACCACTAGAGGAAAGCAATTGGAACACAGAAGCACCAGGTAATCTC
TTTTAGAAAATGTATTCTGAATTGTTTATTTGGATAAATACTGGCTTTAAGGAAATAAATTGTTGGAATGATACTCAGATTTTTTTTTCTCCTT
GTGCTTAGCCTCTGCCATGTTACTGATTTTAAAACCATTTTGNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNTATTATATATTTTGGAGATGATTTCATAGAATAAGCACACAAAACTTTCAGTTTTCTGAGACCA
CTTACAGTTAGCCAGTCACGTAAGGATGTAGAAGAATAAGTCAATAAAACCGCTCCGCCCTCCATTCAAATGGTTGCCTGCCTCTCACTGAACC
AAACACTACTTTTAAAATTCATGGTAGAAAGTCCAAATCCTGTTGTGCTCACTTTTAAACTTCCCCAGCAACAAATTTGTCAGACTGTCACACA
GTAGCAGCTTAAACAAATCTTTCCCTCGCAGTTTTTTTCCTTTTTTTTTTTTAATCATGTAAATTTTAGTGTCACTCTGGGGGAAGCCTGGAAG
TGAAAGTTGTAAACCTTTCCTTTAGCTCTTGGTGAGGCAAGCGAGAGAAAGTGTGCATTGACAGTGAAATGCGCACTCTGGGTGGGAAGGAATT
CCTCATTTCCCTCTCCCGCTGCAGGACTTGCATGAACTCTGTCCAAAGCCGCAAGGACTGCGCGTTCATGGTGCCCCTCGGTAGAGTCCCAGCT
GCGCGCTTGCTGATTGCTATTTTGCACCAAAGACGGGGCGATGATTTTGTTTGTCTGTGTTTTTCAAGTGCGTGTCAAGTGGCTCAGTGACAAC
CCACACAGGTCCAGGGATGCCCGAGGCCGGATTTTTGCAGTTTTTCGTTGCTTTATCCTGATCGAGCTTCGAACTAACTGAGTTACTAGCAGTA
ACTCAGCTGAGCAGCGCTCAAGGTGCGAACCTTGAAAGCCAAACTTGGTGCCTGTGTTCATGTAACTGTCTTTCCTAGGCTTCTGCCAGACAGA
CAACCCTAAACTCCATGCGCAGTTTGAGGTTTGACCCCAACGCGATTCCTTTAATCTTTTGAAGGGCCACAGAGACACCCTTTCCCCTGGAGTC
AACGTCGAGCGTTGTCTCTGCCACACTGTTCTGATGATGCCCTGTCTCGGTTGGTCTGTGTGGATGTGCTTGGCTGTGGCTCCAGCAGCACTAG
ATCTGAAGTCGGCTGCTCAGAAAATGCATTTGAGGGGAGTCTAGCTAGCTATTTGAGGGAGTCTCGCTTCCACTGGCCATCAACTCTCCAGTAA
ATACGGCACACCAGAACCTGGCTGCACTAGGAAGCTGTGGCCCTGGCAGGCGAGCGCAGCCAGCGGGATTCGACCCCTGAATTGGGACTTCTTT
AAGATTCGTTTTAAAAGAAAAGCAAGGAGGAAAAAATGTCTTCCCAAAGCTCCGGATCCCCCCAAACGCCAGGGGACCACTGAGCTTTGCCCTG
CTGTCACGATCTTTGTGCTGAAGGAGGAAAGTCGGGCCCACTCGGGAAAATACAGAAGGCTGGAGAAGTTGAGCCTTCGCTGACAATCGCTGGA
CACCTGAGGAACTCATCGTCCTGAACTACCATAAGGCGCAGCCCAGGCAAAGAGGCTTTGAGAAGAGATGAGACCCTAATTTGGGTGGATCACG
ACAGTAGAGACCCACCAGCCGGGGGATTCCCATGCAAGCTGGACCGGCTGGCTACCACCGGTCTGAGAGACGGGGGCGCTCAGGTGGGTTCTGG
TGGCCACTTCCTAATGTCTGTCCCCAAACTCACAAGATCGACCCTAAGGACCTCCAGGTGAGACTGGTCCTCGCGCCGTCGGGGCGTGCCCAGT
GCCCTGCTCCGCTTCCCACGACCCCTAACCAGGGCCGCTGCTGCCCCCTCGCCTCCGGTCTGAGCCCCGACATGACCGTGAAAGGCCTGCCTGA
TCCTCCATCCCGCCAGCTCCTACCCGCCTGGGCCAAGACAAGCCACCCCAAACTTCAGACAAGGCCTCCGTGCCGCGACCTCCACAGCTTCTTC
CTCTTCCACGGAACCCAGGCTGCGCAGATGCGTGTCGAAGGAAGACACACGCCCGGACCAGCCAGCGACCCCAGCACAGATGAAAGTGGAGGGG
TTCGGTCAATATGTGGACAGTAGCGGGCTCCATGGAGTGGGTAGTAAGAAGTTCCGGAGGAAGCAAGGCCCAAGAGAAGTAAGGGACCCCGGAG
CACAGGCCAGCTGCTCCGGCTGGGGACAGTAGCCGGCAGCGAGCCAAGCAGGAAGCAGACCCTTAAAGCTCTTCGGCGAAAATGCCCTCGGCGC
CGCGGGGACGAACAAGGTGGGGGAGGTGCCCCTACATTGCTTAAGCGGAGTGCGTTCGTTGCCTGAAGCAGACCTCCGGACTCCCGCCGGAGCT
GGAGGGTGGATTCGAGCTCATGAGTTCGGGGTGGCCTGAGTCCCCCATGCTTTCAACTTTTTTTGTAGTCTAGAGTTCTTTGAGGCCTTGGTTA
GAAAACTATCTCCTCTCCAGCCGCTGCGCACACTCAGAACGCGGCGCGCTGCGTAATCAAGCCGCAGCCCAGCCGCAGAGAAAAGACACTAAAT
AAAGCAACTCTGCGAAGGAGAGGATGCTTAAAACCCAAAACTTCTGCGGTGGAGCAGTGGCTGGCCTTGTTTAAATGACTCGGTGCCACTTCTC
CTCAGCTGCTAGTCTGGCCCAGGGAGGCGCTCAGAGCTTCCCCCACTCGGCCATCAGCCCTCCAACCCTCTTGCGGCCGAATCCCACCCTGTCC
TCCCAGGACCCCAAGCCTTGGCTCATGGACTGCAGTGCAAGGCTGCCTAGGGACCATTCCCGGATGCCAGCCCAGGTCCCTGAAGCCTCGCGGT
GGCAGCTTTGTGTCCAGGGAGCACAGGGCGTCTCCCTCCCGCTGATCTCCAGGCCCAGGGCTTCTCCGGACGCAGCCCTCGGTCCCCAAGCCCG
ACCGGTCTCTCGATTCCTGTTTCGGGGTCTCCAGGACCGAGGCCACCAGTGGCGCCGCTGCGCCCGGCAAACCCACCCAGGGCGAGTGCACCAC
CGCGCGTGGCTGCCTGGCGTGGGATCTGAACCTGGGCCTGGCTTCGGGCAAACATGGCAGCGTGTGCCTGTGGGTGCGGGGTCGGCGTGGGTGC
GGGAGCTGGGGACGGTAGGGGCTGCCACCGAGTCCCGGGCCGGGCAGCGTACAGTAGCGCGCGGGGGCCCGCGGGCCAATTCCACGAGCGCGGC
AGCCGGCCGCGCCCAGGCTGATACCGGAAAGGCCTGTGATTGGCCGTCGGTCTCCTCTATGCAAACGAGCTGAGGGAATGCAATTCCTCGGGTC
GGGCTTACAGTAAAAGCACGTTCATTTCCAGGCACTCTCATTCATAGAGCCAGCGGGCGCGGGCAGGACGGGCGCCCCGCGGCCGGACCCAGCC
AGGGCACCACGCTGCCCGGCCCCGCGGCTGGCACTTCCATCCTGAGCTCCCCGAGACGCAACAAACTCACCCTTTGCGGCTCTTTCTAGATCTG
CGTTGGACCTTCTTGTTTTCCTTTCCTTTCCTTTTTTTTTTTTTTTAATTTTTATTTTTATTTTCCCTTTCTTACTTTTGAATGAACCGGCTTC
TAGGCTTTGGATGTTTCAACTTCTACGCTGTCAACTTTTCTCCGATTGTTTCTTTTGACAACTGCGGGAGAAAGTGTTCCGGAGTTCCAGGCGA
GCGGTGAAGTTGCGTGTGCGTGGCAGGGTGCGTGGGAGTGAGCGTGTGTAACTCGAGCCTACTGGTCTACGGTGCCTCCTCCCAGGCCCGCTCC
ACCTGCTGCGGTTACACGGTGCTCGCTCGTGGGTATTTGTGCGCCCCAGCAGAAAACCAGCCACCGCCGGGCCACCTTCGAGGAAGATCATCTT
GCCAGACTCTGGGCTGACTCACTCAACCAATGTAGACAGTGGCTGACTTCCGAAGAGTGCGAGTCTGCCTGTGTGTGTGACCCAACTGCGGCTC
AACTCCCACCAAACCACAGGATCAGCCACAAACTTAACCAGCGTTCCCAAACCAGAGTCCTCAGCTGTGQGAAGCTCTACCCTCACTGTCATCG
ACTTTGACTTTTTTTTTTTTAAGATTAGCTACAGATCTTTGTAAACCACTGACCATTTATTTGGCCAGAATTTATTGAGTAATGACAAGTGATC
CTATCCCAGCCTTCCAACTACTGAAGCTGATTTTCAAGGCTACTTAAAAAAAAAAAAACTGCAGCAAACATTAATGGATTTCTGTTGTGTTTAA
ATTCTCTACAGATTGTATTGTAAATATTTTATGAAGTAGATCATATGTATATATTTATATATACGAGCACATACATTAGTAGCACAATCTTTTA
AAGTTACGGCTCTTGCTTTTGAGAAACCAAGTGAGTTTTTCATGGTAAGAGGCGCGCTCTGTATCGAAGACACTCCTAAGTTTTGTATTTTGTT
GAGACTCTAAACAAAACTGACCCGCAAGAAAGAAAAACAAACTGACAGGCAACTAACTGCTGGAACTTCCAAATCTAGTTTTTGCTCGTCATTT
TTTGGTTCTTGTTGTTGTTCTGCGCATAAATATTTTAGGACGCGTATGGGAATTTTGCCTCCGGGACCGTTTGTAATAGCCAAACACTGAACTT
CAACTCTCAAAGCGAGGCTCTGTTGGGCATTTGACTTTGAATTCTGGTTGCCCACTTTTTAACGCACTAAGCGGCCAGTTGCTAACCCTGCTTG
GGTGTGAGGCCGATCCGTGTAACTACATCTCAGTCCCCGACTGATCCTAATTTACTCTCCTACCCCCACAACCCTCCGGTAGTAATAAAGGCTT
CTGAACTTGTATGTTGGTCTCCCGGGAGCAGCTTGCAGAAGATCCGAGCCCCTGTCGCCGTCTAGTAGGAGCTGTTTTCAGGGTCCTTACTCAA
TCGGCTTGTTGTGATGCGTATCCCCGTAGATGCCAGCACGAGCCGCCGCTTCACGCCGCCTTCCACCGCGCTGAGCCCCGGCAAGATGAGCGAG
GCGCTGCCGCTGQGCGCCCCGGATGGCGGCGCCGCCCTGGCCAGCAAGCTGAGGAGCGGCGACCGCACCATGGTCGACGTACTAGCTGACCACC
CTGGCGAGCTAGTGCGCACCGACAGCCCCAACTTCCTCTGCTCCGTGCTACCCACTCACTCGCGCTGCAACAAGACCCTGCCCATCGCTTTCAA
GGTACTCCTGCCTCAAGAGGGTGGCGGGAGGAGCAGGTGGAGGTCAGCCGATGGCCAGAGTGAGCCTAGGGGGCAGAGGCTAAGGCGGCTTCTA
GAGAAGGGTGCTGCCTCTAGTAGAAGTCTCTGGTCCTTCTCACTGAGTTTGGGATGGGGGGGGGGATGCTGCGTTACCGTGGCGACCCTCGGGA
GGGTCAGCCTCAGAGGTATCCAAGAGGGACTTTTTTTAAAAAAAAATCAGGATAATTTCACAGCGCCTACCAACCGCCTTCCTTATCTTTGCTA
CGAAATAGAAAAAGTTAGAACCTTGGATCCCGCCAGTCCATCTTCCTTAACCTTCCCCGAACCTCTGCATGAACACCCAGACCCTTTTGTATCT
CTCAAATGAAAGCATCAGGAAATAAAATAAGAGATGCTTTAGTACCTTCAGGTCAGAAGAAAAAAATAAATAAATAAACCAAGGGAAACAGCCA
GGGATCACACACACACACACACACACACACACACACACACACACACACACGCCCCGCCGCCTTTTTTTTTTTTTTTTTTTTTTAAATCGCGCTA
TAACATTCAGCGTGTCCAAAGTCCGCGGCAAGGAGTAGTTGGTTAAGTTTGCCAAAGTCCGGAGTTTTTCCGAGCCTGTAGCCTGCTCCTGCCA
GAAGCCGCAAGTATTGAATCCTGTTGCTTTTTCTGGAACCTCCAGAATATTATCGCTGGCTCACCTAAATTAAGCCCCAGGCAAGAAAGGTGAC
AAGGTGTCTACCGACACCCAAAAAGGTCCAATCCAGCAGCTTTTCTGTTTGAGCAAGCGGCCTTGTACACCCGGCAACTTCAGGTGGGCAGCTC
ACGTTGGTCAGGTCGCTTTTCACCCTGATCTTGCCCTAGCTTTGTGTTGCGTCTAAAACTTCGCTTTCAGAACCTATATATAAACAACCTCGGG
CGGTTTAAAAAAAAAAAGAATAAGAAGAAGAAGAAGAAAGAAAGAACCACCCTACAACTCTGGCAGGGAGCTGGAGGAATTTAGATTCTCTTCT
TTGGTCTTCTCCTGAGGTGGGTTTTAAAAGGTACAGCTGAGAGCAAAAAGACTCTGTCAAGTTGTGTTTTTAAAAGGCTAAGAAGGCGGGACCC
GAGCTGAAAAGGGAAGCTGCTCTCCTTTTTCTTTTTTCTTTTTTTCAATTTAGCTGAATAATTGTCTCATTTAGGAGGGAGATTAATTTAATTT
AAGCTTTTCGTAGTTCCAGGCCTCAGCTGGATCTTGATCTAATTACAATGAATAGCGCTCACTTTTTGACAAGTGCTGTCCGGTTCCCCTTTTC
ATGCTAAAAACACCATCCACATTAAAAAAAAAAATCATAGAAGTAGAATTTCCTCCTGACTCTCTCTGTACAATTGCCACCTTTACCACTGTTG
TGAAAGCAACTTTTTAAAGGGGTAGAGGTGGGGTTGAGAGTCTGAAGGGGAGCCAGGCACTCGGGTTTTCCTTCCCTTCCAAGTATTGGGGAAT
CCGTCCCAATTCCTTTAGAAAAACAAATTTAGCTGGTGTTGTCTTCTGTTTACCCTGAGAGGGACCTATCTGGGCTCAGCCCTAAGGTCACTTT
CAGAGAACAGAGAGAAATCAAACTTATTGTAGTCAAGGAAAGGGCCAGTTAAGGTAGATCTCCCTGCCAGCCTAGCTAAAGGGACTGTGGCTTC
CTTGAGGGAAGAAATTGGGTCTTTTCTGAGCTTTGTTGCTGTGGTTGTTGTTGTTGTTATTGTTGTGGTTGTGGTTGTTCTTCTTCGTCTTAGT
CTACCAGGGAATCCCAGTTTATCAAGACACTAAATAACTGATTTTTAAAATCGTCTTCAGGAGTTGACGTGGTCCCGTCTTGAGCTACCACATG
GAAATGTTTATTTTTCTTTTCAAAAGAATTAAAGACCTTACACATTGCCTTTACAAGCCACGAAAAATAATTTTTTCCTTTTAACAAAAATAAT
CACTGCCCTAGAGATTCCCCTTTACAAATGATTTTGGAAAGACCTCAGTGTGTTTCCCTGAGTGACAGCCAGGAGCCATGACAAAGGTCACGCT
GGACAGGGAAGTTGAAAAATCAGACCTCTGGATTAATATCTGTCACCACCTATGCTGTCCCACAGAGGGACGGCTGCTTCAAGGAGCTCACCAG
TCAGTAGATAGAATTTGCGGAGGGAAAGGAGATTTAAAGGCTGGGTTTAAGAGGAAGCTCGACTTCGAAACAGACCATCTTCCTTGCTGTATTT
TCTATGGCCCCTGGCCCCTCTGTGTGCCAGCTCCTACCAAACACACACAAGCAGTTTCTACCAACAGTCTTTACATTTACATGTGAATTAAGAT
TCCGGAATCTCTTTAGGGGTTAACCCTGCAGGACTTATCCTATTCCCTGCTGCCGCCACCACCCCCAGATAAAAATCGGATGATGTGAAGCCCT
GTTCAGATCACTTGTAGAGAAGCATCTCCCCGAACCTTCCGCAATCTTGTTAAGTCCTCCGCTTTAATCTTCAGAATTATCAGGAGACTTACAA
TCATGAAGTCCCTTGAAAATTGATTAAAATTATGCTAATTACTCCTCCCATCCTCCACCCCTCTTTCCACCTATAGACTGTTTATCTCACCATA
GAAGAAACACTTAATTTCACTGCCGAGAAACTGGGAAATCTGCTCCATACTACACAATATTTGCAGATACCGTCTACAAAGTTGTCTAGGTGAC
TTGCAGTAGATTTTATAATACTGTAATAAAAGAATTTGTTTAGCCCCTGGTACACCAGAGCACAAAAATATAAACATACCTTTATCAGGGAAGT
TCAAAGACCATGGGTGCTGATGGAATATGCTTTCCAATAAATAAATAAATAAATAAATAAATAAATAAATAAATAAATAAAATTCTGGCCGGAA
GACCATCATCCCTAGCTATGTGTTAAATGTCATCTCAGATAAATTCTACGTTCCATTCCACAGCCTCTTTGAAACTCTGGAAGATTAGAGAATT
TGCAGTAACTAAGTTGTTGCTTTAATTTCAACCTGTAAAATTGTTGGAAAATGCCAGTTTCAAACTAAGAATAGGCTAGCCAAGAACAGTGCTG
GGAAGTCCTCAGAGCAGAGAAGACAGCTGACATTTGGCCCAACTTCCTGATACTTGAAGAACAGGAGAAGTTCAACAAAATATGAGCTATCTTG
CGCCAGCAACTCCCTTCTTGTCTGTTTGGTCTGTACTAAATGTAAATGTTTCTCTTGCCTTACCTATTGTTTATAGCTTTTTACTGCAGACACT
GCTTTGTATTTCTGTTTGAAGTTTGATTTCTTTTCATTATTTCTATATCATCTCTCAGTGACAAAAATCTATATCAAATTGGAAGACATCTTAG
CTTTAAATAAAACAACAACAGTGCTAGTAACCTAGCCCTAAGTGTGCTTGTCATTGTTTTAAGTACAGAGTTAAATGTCCCTGGATTTAATTTT
GTCTCCTTCCCTTCTCCATCAAAACTCTTTGGAGATAAATCAAAAAGCCCAAGAAGTAGTAACCGAGCCACTCGATCCCCCCTTCCTGCTTCCT
TTAGAAATGGGGGTGATGCAATTTAAACTCCTAATTTTTAGACTTTTGAATTCCAACATCAACAAGCTTTTCTTGCCCACCATGGTCCTCTTGT
GCTGTACACGAGGGGGCCTCATGGGTTTGACCTCTTGGTAATGAGGGATCCCTGGAACAACCATGGTAAACTGAGGCACGGTGGGGCACAGTGG
GAATTCATGACTTGGCGTGCCTTTGGGCGCTCTGGAACAGGATTTATTCATTTTGGCCTCTTAGGAGATATGGGCCCCAACAGTCAGGCATTCA
GACTGGAAGCCCGGATGTCGATTCAGTGCCCTCGGAGCCCAGTGGGTGTTGAGATGAACTGGAAAGGCCTAGGTCTCCACAGGGAGATGAAGGC
AAGGAAGTGAAGCATTTTCACAGAGGGTATCTCATCCTTGTGAGCAAACTGTCCACAGGCAAATGAAAACATTGATGAGAGAGATGGCGGCAAA
AATAGACAGAAAACAGAATTTACAGCAGTAACATTTTGTAGGAATAGAAAGAAAAGATAATAATGATCAATTAACCGAGCAGCCGAATTTATTT
CAATGATGTTCTTTGAAACAAAAGAGTTGTCTTATTACCACTCCTAATTAAAGGCATCAAACATTTGTGGTGGGATAGAAACATCTAATTACAT
ATTGCTTGGAGAATAAGCGTTTTTAAAATCTCCTTTGTAGAAAACACGTCCAACAGTTGACATGAAAGGGTTAAAAGCAGAGCCAAGGGGCCGC
AGGGTCCTTGTTTCACAAAGCAAGTGAGTGAAAACAGATAAAAATTAAAGAGATAGCAAGATACATACCCAGTCCTAGAGAGCAGAGTGTCCTA
GTGATGTCCTAGGGGTCCCCTCTGTCCATTTCTCAGGGTAGCTGGGGCAATTTCCTGACATACGTTCTTTAGAACCCAGGAGATAGAGCAGCCA
CAATGAATAAAATCGCCCAGATAGCCATCTGCAGGACATCGAACAGCGGGAAGGAGACATGACTAAGCACCCAGTAAGAATGGCGAGCAGAGAG
ACCAGACCTTGATACCAGATTACCACATCTGCCGCCGCGTTATTTCATACAGAATGAGAAATATCCCTCTGATAGATTGTGAACCAGCCAGTGT
ACAAAAACTGAGAAAAACAAAGTGGCAGTAGAAGAAGAGATGTTTCTGCTGCCATCCCGGGAAAACCTGCCCGAGGATGACCACACAACTGAGA
TAAATAACAGACATCTCACCGAAGACTGACAGCATGTGTCTGGAATGGTTGGAACAAACTGAACTCTCGGGGCATGGCGATTTGAAATCCTCTG
GTCGCTTTGAATATGGAACAAGGCAAACATAACCGATATCAAGTCAAAACTGGAAACTTTAGCTGAAGTCAAAGAACGGCAGGCTTGGTGGTGC
ACACCTGTAAATCCTGACCTCGAAGGCTAAGAGGACTGGGAGTTCAAGGCCATTCTAGGCTCCATCGTGAAACCATAGCTCTCTAAAACAAAAC
CAAAAGGACCAAAGAGCAGCCAAGCAGTGGGTAATGTTTTGTGAAGCACTTTCAAGGCTTCAGAGTGTTTTAATCTGAAATTACTACTTTTTTT
GGTTTTCTGGATATAAAGTAAGCTCATCTACAAGTTGTCTTTCCAAATATACTAAGCCCTTACCTTTTCAGTGGCAGTAACGGAATCCCCCAGA
ACACACTTCATACAGATGCCAGACAGCACTCTGGTTCTACCTTTGATTTGAGCAGAATGACCACACTGGAGCACCCGTACTCACTGAGGTCATC
CCGCTTGTAATTAATAATAAAGAAGTGGCCCCTGTCTACTTTTTCTCCTTGGGTGCAAGAGTTTGCTGCTGGTGAGTGTCCAAGAGCGGCACGA
AGAGGTGCCAGAGATTAGCTGCAGACAGACAGTTTAAGTATTTCCGGGTCATCCTTGGAAGCTGCACACTGGGTGGCACACACACCTCTAACCC
CAGCACTAGCAGAGAAGACAGGAAGATCGGGGGTTCAAGATCACTCAAGGGTTCAAGATACAGTGAGGCGGGGGCCAGCCTGGGCTATCTGAGA
CCCTGCCTCAAACAACAACAACAACAACCAGTAACAAATTTAGAGCAGAACTAAATTTAAATACTTTCATCCAGGTTTCCTTAGGTGCACCAAG
AAGGGGCCAAGCAAGTAAAAGGTGGGGCCAGAGAGATGGCTCAGTGCTTACCAACATGATTCATGATACAGAGAATGGGTCTGATTCCTACAAC
CTATGTCAGGTGGCTCACATCTGCCTACAACTCCAGTCCCAGGGTAATTTGCTCCTCTGGCCTTCATGGCCTGCTCTCAAGAAAATGCCCACCC
ACATACACTTAATTCAAAATGCCAAGATAAATCTTTTTTTGAAGTGGATTAGATCACAAATCTACAAACACAAGACTTTCTGTCTTTCGAATAT
GAGGTCTTAAGAGATAACTGTACCAATGAGAAACAGTAGTAGCTTTTCAGAATTCCTGTCCCCGCTTGTCTTGTTCCCACTGTGTCCTGTCACC
TTCTGTGGCTTTTCTCCTTTAATCTCTGTGTCTTCCTTATTTGAAAATCCACACCTGTCTCTGCATCGCAGGACTCACAGCAAGCTGTTTCTGT
CACGATGATCAAATGATGACGGACGGCTCTTCTATAAACTAGAAAGTGTCACCATTAAGCATTAAGTTTAACATCAGCGCAATTGCCAGACAAA
TGACACAGCCAGGTGCCTGTGACAGCACTGATGTCTGCATTTGTCCCTTGGTTGACGTCTAGGTGGTGGCACTGGCGGACGTCCCGGATGGCAC
TCTGGTCACCGTCATGGCAGGCAACGATGAAAACTACTCGGCAGAACTGAGAAATGCTACCGCGGCCATGAAGAACCAGGTAGCGAGATTCAAC
GACCTCAGGTTTGTCGGGCGGAGCGGTAGAGGTGCGTATCTGTCACGGATGGGTGTGAAATTCGAACCCGGGTTTTCCAAATAGTAGCCGTTTC
TTATTATTGATGCAGCATAATGATATTTATTTAAGGTAATGTTTATTTGATACCTAACAACTGGATATTATCACGTTTTGATGACTAAATTGGC
TTACAACTGACTTGGAACGCACAGCGTGTGATGGACTTGCAACGCACAGTGTGTGATGCGTCAATTGTCAAGTTCAATAAAACATTCCACCGTT
TTCCTAAACTGAGATAAGTGAGCGAACCACCCCAGAATGAGGACTCAGATTCTATAATCGGTGGTCACTATGCGATAATCAAAGCCATGAGCCT
TTATCATATGGTACTTACGCTCCCTCGAACATGCGACAGGGTTTCAGTTGGTCACATCAATAATTTTGGCGGAACCCTTATTGATATAGTTTTA
AGTAAGCTTATATTCCTAGGATTAAAAAAAATGGACTCTTACTGGTACATCTGAGAGCAGTGTGACCTTCACTATGCATTCTTTGTGTATGGTT
TGGTGGGAGAAGCCAGGCTCACTTAACATTTAGATACTGCTCATGTGAGAGACCAAATTATTTCCTTACCCCAGCTGGACATGCAGTTATTATG
CCTCACCTTTAACCAGTTATTAAACTATTGGAGTAGAAAGAGGACCTGCTCTCACTGTAGGCTTGTCTGCTTTCCAGTTATCTTCCGAGGTATG
TGTTCTCTCTTCATTTCAAGGAGCTACGGCACATTTTACAAACATCAGCAGGCCGTGTGAGAGTCTAGGAGCAAGGTCTTCGCTTAAACCCTCA
CCTTTCTAATGTAGAATTGAACGATCTTAGCCGTAAATGATATCTCTAGCTTAATCAAAGCGGAATGACATATATCTAAGTCATACGATCAGTT
CTCAGAGAGGGGAAGACACAGGGAGGGGCTAGTCGTGTCCATCGCTCAGTGGTAGAGCCTTCCGTGTGAAAGGCCTTTAGTTTGATCCCCAGCA
CCAACCAAGCAAAACAAAGCAGGTAGACTGGCAACAGACAAGAAATGTCAAGCAAAGGGATTGTTACTACTCATTCTCAAGCCGTTCAGAACAG
TGGAAATTCCGTGTTCATACTGGGAAAGAGGAGAGAGTTGAGAGTGTGAGCCTGGACTTCTGTGTTTGCTCCCCTACCCCTGCCCCCAGATGCC
CAGGGCATTTTCAGAATGATGTCACAGCTGGAGAAAAAAATGTAGAGAACATATAATGTTATTGAAAATATGTGTTCGCGCTACTGTGCTAATA
ATAGGGTGATTGAAAGAAGAGTCTACCCACTTATCGAAGGCCTGGGAGAGCCTGCGCCCTTAAGTGGATCCACTTGGAATATTGAAAGTCTGCC
TTTCAGCAAATGCAATTCGCATTACGGTTCAGAGGCATGTGTTTAGAAAATATCACCGAGGTTTCAGATGAGTCGCTGGCCGAGCGGGTGGGAA
GCCAAGAAGAAAGGAAGACCCCAACAGCCAGGCCCACGCCCACCTGCCTAGCAATAAGCTTAGTTCGTAGGATCTAGCTGGGTTGCCACAGAAC
ATGTCCCCATACTCCTAGGCGTCTAAGTGGGTCTTTTTGTCATGTTTGGCTATGGAAGTTTCGAAGACTATTTAATGGACTATTCCAGTGAGAT
TTTCCCGTAGGGAGTTGCATGGTGGCTTTGTGACGACCGTGGCAGCCAGTTGATGATGATCAAGACTGCCGTTCTCAAAGCAGCGTGCCTGCCT
ACCTTGGTGGTGCTATGTGCTGGTCGCCTATCCCTCTTTGGCCATTTCTCTTTTGAGTGGGTGACTAAGAGGTGCTCCTGTTTACCTGAGGAAC
AATGGACTGTTTCCCCAAGGCGTGGAGTGTTTGCACTCTGCTGTCTGATTCGTGCCAGTGTGCACTTGAGTTTGCATAACCGCCCCCCCCCCCC
AGGAAAACAGCAGCACCCAAGGCGAGGCTGGTTTCCCATACCCTGGAATGAAAAAAATTAATCAGATGCAAAAAATTGGATTATATTTGACTTG
AGCAGGCTCTCCTCTTCATGAGGGGTAACGAGTAATTAAAACTCAGGACGACTAATTGACCACTTGACCCAGTTATGGTTTCTATATATAATGT
TGGTCTTGAGTTTTTATCCTTTAAATTTTTCATGTCAATACCAAGACAAGTGAGCTGTCGGTGTAATAAACATGGTTTTATCTCATAATTTCAC
TTTGTAGTCACAAAATATGATTTTATTTTGATTTTTCATCCCATCAAAGCCTGCTGGATATTCCCCACCCCACCCCACCCCACCCTCTCTCACT
CTCTTTCTCTCCTTCTCTCTTGCGATTTGCTTCCCATTAGAAACTGAAGAAATGACTCTCACTAATTCTCACAATTAGCCCTAATCTCCCTGGC
TGAACCTTATCCTTTAAAAGTTGCATTATGCTTTATGGAACGATCTTAAGACCCGACTATTTCATTTTTCATTTTTTTAAATGTGCCTAAAAAT
ATGCCCTCAGAGGCCACACACTGGCCCAGAACGTCCTGGCCTTCCAACACATCCACATTCCGTAAGGTCAGTTTGGGATTCTGTCAGTGATGGC
TTCTCGGAGGGCCCTGGGTACTCCGTGTGGGATAAAGGGTAGAGATGACATCAATGGACACCCTCCTTTCTTGTTACCAGATGCCAGATTTTGA
GGATTGTGACCCTCACAGAAACTGCCAGTTAACCAAGGAAGGATTTTTCTCTGTTCCAAGAAATGGATCTGTCTGGGAGCTGGCTGATGCTCAG
CTCTAACCTCAGGTTCAGCCTGTGACTGGGAAAACCTGCCTGTCTGGCTTCTAACCCCTGGCTGCCTGCTGGCCCCTGTGAAAACCTGGTGAAG
GCAGAGGAAGCTCCTGCCTGCTTCCTCCATGACCATGGCTTCCGCCTGCTCTCCTTTAGATCAGCTCGGGTGTATCTCCTCATAGGAATGTTTC
TCCCTTTGATTCCCAATCTGCATGTTGATTATCTGACGGCCTCTACAAAGGAAGCAGAGATAGCTGCTCCCGGTTGGGAGTTTAGGGTGGGGTG
AAAATCAGAGGGGATCTTGGGAGACCCTCTCTGAAGTCCAGAGATTAGGAGGGCGCTCAGGAGGCTGCCCAGAACAATGCATGCTTTAGTCACA
GGGCCTGAGATTATCACTCATCTCCGAGTTCAGGGTCAAAAAGAGATCTCTTTCATGAGGCAGCTCAATTACCTCAGCCTTTTTCTCCGTCTCT
AACTTGCTCCCATGAGCTGGGGTGTGAGGTGACAAACATTTAAGAAACCTTGGCAGTGACATCTGCAAACACCCATTTCCTGCTGGGGTGTTTC
ACCAGCTGCCGAAGACTTGATCCATCAGTGAACTTAGCTTTAACGATGCCTGTCACAGGGAAACAGATCACGCAACTACCAAACATGACAACAG
CCGTGTGGCAAATGTGAGGAGGGAGACCACAGCCAATGCTCACTTGTTCCAAGTTGGTGATTCTTTAAATCTCTCGAAGCTGTCATAGATAATT
CTGAAAATCCCCCCAAAGATTTCCTAAGGTCCAAAAAAAAAAAATGCACACGGGCCCAGATTTCTATAGTCCAGCTTAGTTTCGGGATTGTTTA
TGGATCTTCCTGGCACGTGCTGATACTAAAACGTGCGAATCTGGAACGTGTCTTGGACTGCTGAGGGTCAGTTCAATGCCAAGTCTTAAACACA
GCCCATACTCAGAAGATAAATATACATGAGGACCCACAAGGTTGACATTTTCCTGTGCTTCTCTGTTGTGTGGATGGCGGTGATATCTGTAGAC
TCAGGACCTGGTTGGTAGAGGGTAGGAAGGTATAGCAATTTAAAAGGCCAGGGCAGGCATCCGGTGGACCACTAGGCATATGGCTGGATGTAAT
CGTCTGTGTGGGAGCGAAGCCACCATTGCCAGAGCAGTCTCTTCCCTAGTGACTGTCAGTCCTGGCTGCAGTGACATGCACGCACATCCCCAGA
AGACCAGTAGTCTGCTGCTTGCCAGGAACAGGTCTCCTTTCTTAGTGGAATCTGCTGTTTACGCTGACATCCTGGCAGCTCAGAACTTCCCCAG
TTTGTGCTTTTCTGAAGTGAAATTCCTTCCTGGANNNNNNNNNNNNNNNNNNNNNNNNNNNNAATATTTATCCCGTCACAGTCTGGGCTCCTTC
ATATTTGTGTAGTGTCGGTTTCTTTAAGCATTTCCATTCCGCTTTTACGTGATTTGGGGTACAGTGTGGGATGTTGTCTCTCTCGGCTGAGCGG
AAGGCTGTCGTGGAGACGCGTTGCGGGAAGGATAAACTGTTCTGGCGCTCCAAAAAGGGAGGGCCGCCTTGGTGTGCGGCTTTTCCGGGAGAGT
TGGTCTTAGTGACTCATGATGTTATCACAGGATCCATATCATTCTCTATTTGATCACAAATTCATAGAGGAACAAGAAATCCAACTGCCACCCC
TAGGGATGTTTCTCCGAGAGCAGTCCATGTAAGCACTAGAAACTGATTGTTGGGTCTCCTAGGGAGAACAAGAAGAAGGAAAAGGTGTGCGCTA
TGTTAAAATCCAATGTCCACTTTTCTTCCATTTTCTTTCTGTTTTTTTTTTGGTTCACGGTTTGTAATAGCGCATCTAGTAAAATCTGTTTTCT
TCGGATTTAACTACAAACAAACAAAAATAAAATGGTACAAAACAAAAATAAAAACTAAGCAAAAGCCAACAAAACTAAGGGAAGCCTGGGCCCC
TCAGAGAACAAGCCCCGGCCGGTCCTAGCAAACACTTCCTTTCCACAGCAGGCCAACAAGCTTCACCCCAGAACCAACAGCACTGACTCAGAAT
CTGCCTGTAAGGATCAGGTTTCCCCTTCTCTTTATTCCACAATAAGAGGGTTAGCAACACTTTCCTCAAGCAAGCTTCTCCCTGTCCAGCTTAA
AGCGTGGGGTGGCCGGAGCCTCAGCAGGGACCAGGAGCTGCCCGCTGGACGCTGTCCCTGATGGAATTTGTACTGTCATGCTCTACCCCAGCGG
GCTTTACAAAGTGCCAGGCATGGCGGCAGGTAGCTTTTTGTAGCTGAGGTTATTTGCATGCGCAGGATTTTTCCCATGTGGGGCAGAAGCAGCC
CTCCAGCCCTCCTATCGAGCCCCCTCCATCCCTTCCCCAGCAGCTAGGCTTTCCTTATGAGCCAGCCCTTTTTGGTCAGGACTAAAGACTATCA
ATCACAAACTACCTGTGAGAGGAATAATGGTTGAGTCACTTTTAGAATTTCCTGTTTGACAGCAGCATAGACATAAACAGTGATCCAGCTAGCC
ACTCAGTCAGAATAGTTCTAACAAATCAACCTGCTACCTCGCTCTTTCTTCTTCTTCTTCTTCTTCTTCTTCTTCTTCTTCTTCTTCTTCTTCT
TCTTCTTCTTCTTCTTCTTCTTCTTCTCCTTCTTCTTCTCCCTCCTCCTCCTCCTTCTCCTCTTCCTCCCCCTTCTCTTCCTCCTCTTCTTCCT
TCTCTTCCTCCTCTTCCTCCTTCTCTTCCTCCTCCTCCTCTTCCTCTTCCTCTTCCTCTTCTTCACCTTCTTTTTGGTACTGGAAGTAGGCTAG
TTATAGAAGAATATATTCTGTTTCAGGATTTTTTTTTTAAAGAAAAATTACTTGTTGAAGCAGACAGATGGTGTAATCTCTCGTCCCTGTCTCC
TCCCTGCTGTTGATTATAAAACAAGCTGAGAACAGTAGGCGGTGTTTAAGAATGACTGCGGAGTCACGCTCGATGTCTATGGAAGGTCGGTCCA
GAAACCCCCTCCATTCAATTCAGACATCTGCAGCTGCTCCAGCCACTTATACAAAATCGCACAGCCTTAGTAACTAACCCTCAGGACACCCTCT
AACATAATTTACATCACACCTAGATTGCTTATGAATCTAGCTAACAGAATATCGCTTGCACATTAATAGTTCCTGTGGTATTGTTTAGGGAATG
ATAATAAAAAAAAATAAAAATAAAAGAAGTCCACATATATGTTCAGTACAAATGGATTTCCCCACAGATATTTGTGATTGCAACTTCTTGAACC
CAGGGGACCCATGGACATAGGACCGACTCATGGAGTGGTGTGCTGTTTCTAGACTCTGTTGGGCCGTTAGGCAGAGGAGCCTCTACTTTCATTG
AGAGAGTTCTTGGACTAAAATGTTCTTGAAGCCCCAGTGCACATGAAGCCCCAGTCAAGCAATTAACAAGAATGGGTAAGCAAGCTGTCTGTGA
AAATGTCCCAGCGTGCCAGCCACCTATCAGGGAGCGGGTGGGTAACGGTAATCTAGCTGATCACTTCATGCCTGTGTTTGTAAACATGCACTAG
TGCGTTTCAGCCAACCCCTCTATGTCCTCCGTGCCTGGCTCTGCAACGAATCTCACATCCACACATGTTTGGGCCTCTGTGGACCGTGGAGCTA
ACAGTACCGTCCAGTTTTACTCTGCACTGCGTTGGGGTTCCAGCTTACAGCCCAGCCTCTATTAGCCTATATTGGACAGTCTATCCAGTTTGGG
AACTCTATTTCCTATGCCCAGACACTCTCCAGCCAGCGAGCCAGGCTACATGGGTCCCCACGAGTGTACAGAAATCCCAGTGGTCTCCTGTCAC
AGGCTCAGGCTGCAGTGCTGCAGCCCCAGCACGTTGCCTCTGGCTTCCTAAGCCAAGCAGCAAACACTCAGGAGCCACGCAAACTCCACGCCTC
TCACAGAAGATGGAAGTGAATTGGAGCCAAATTATCTGACTCATAGTACTCCATCCTTTTAGAAACTTAAGACCCTCTTCATGAACACATGGAC
CCAAAGGTACCACCTCCCCAACACCTCATCCCAGGAGGCTCAGGTCCAGAGAATGGGAGGCCTGGCCAGTGTCCTCTAGAGAACTGTGTGCCTC
TAATCCAGTCACTTTATGCAAAGGAAGGACTTCTATGGAATTCATCCGCATCCGTATCGCATCTTTCTGGATAAGACAAGAAGGGAGGGACTGT
CTGGAGGTAGAAAGGACTCTTGTTTGATTACAGTATTGGCTGTGGGTCTTCACAGACATTTTTATTTCTATTTGAGATGTCTGTATGGAATATA
TTCAGCCTCTATCAGTTTCTGCCTCTTATTAATGAGAAGCTATCTGAGTCTGGATCATTCATAAGGCTCTTTGTCTTTTTAAACGATGTCTGCA
GAACCTGCATGCAGTAAGGGCCCCATTTTCATCCTTGTATTTAAACCACCCAAGAGGCAGGTCCCCTTCCCTCTTCTTTCCTAGGTCAGTGATT
CATCAGTTTTGATGAGCCTCTTGCTTAGTTTGTCTCTGCTGCGATGACTTTTGACCCCTAAGGGACATTTAATGAGGTCTGCAGATGTTTTTGA
TTGTCACCCACGGAGCCTGAGGGGTGGTCCCTACTATCCAGTGGATAAGGCCCAAGATCTGCTAAACACCCTATTATGTGCAGAATGGCCCCTC
CCACCCCCAACAAAGAACAATTCTAAAACTTTAATCGTGCCAGGACTGGAGAAAGCCGGACTTGACCCAACACTTCCAGAAGTTGTTAGATATC
TATCTCCCTTGCTCCTCCTCCTTCCACTCTTTTCCTTCCTTGAAAAAAAATCCGCGAAAAATTTTAATTCATGGTATTCACACTGAGTCAAAGA
GGAAACAGACAGGAGGCTCAGAAGTTCAAGATCACCCTTGGCTTTGTGTTGAGTTCCAGGCCAGCCTGGCGTATAAGAAACTGTGTGAAAAAAA
CAAACAAACAAACAAACAAAAAGAAATAGTAAATGAATAAGAACGTGTCCAGAAGCCCAGACGAATGAGCTTTCGTGATATCTCCCCCCCCCCC
CCAGAGCTGCCCAGTGGTACCCTCAGTGGACAATTCCTGCCTGGCAGGTAGGCACTCACTCACTCCTTAGATCCCTGCTTAAATGTTCAACTCT
AGGCGCTTAGCACTGTTTAACGGCTCTAGGCTGTAGACCTTACCATCTTCTTAACAGAGAGCAGAGAGGATGTCTCCCATTGACACTTAAGAGC
ACCCATTTCCTTGAGGATGCCTTTCCATCAGGGGAAAATTGGAGAATGCTTGTGTCATGCTTTGTTGAATGTTTGACAGCAACCCCCACCTTCC
TCATGGTGACAATCTGAAATACCTGCAGACTCTATCAACCCTCCTCGCCTGAGGGTGGAGAACTTCAGAACAACTTCCCTCTGTGCCTGTCGGC
TCTCTGCCGCTTTTGCCCAAGAAAGGAGAAGGAGGAGCCTTGACGATCGATTTCAGAGATAAACGCTGGATACTCGTTAGGTTATGCCTTCCCT
GGGTCAGAACAAGCTGGCTCAAGTCTCCATAATGGGTCAGGTCTCAGCAGGGACAGTATCCCTAGAGGAGACAGGGACATTGGCAGGGTCACTG
ATAGACCTGTTTCTTGTGGCTCCTTGTGGATTTCTGAGAGAGAAAGCCTCCTAGCCTACCCTTCCTGCCCAACCAGCCTCCTGGAGCTCTGAGG
GCCCGAAAGGGTTATATTCAGCGGGCACTGAGATTTTAGATTAGCAATGCGAGAGAGGAAATTAAATATGGACTGTAATATGAAGCACTGTTTT
TCATGGGAAAAGAAGTTCTCCCAACTTAGCCAGTCCAACTCTTTTGTCCAACCGTTAATAGCATGCAAATATTTATGAATCATACATGCCCCTT
CTAATTCCCGTGTGTATTTTCACAATAAACAGTGCGTTTCGACAATGGGGCAGAATAATCCCTTTGTCCCGTAGCACACCTGTGCAGGCTGGCA
CCCGGCAGCATCGCATTCTTCCCGAGTCCTAACTCCTGTCTCCACCCAAGCTTAGGGGAGGTTTATGTCTGTCTGACCCCGGTGGACGGGCTCT
GGACGGTCCTCCAGGCAGTGCTCCAGGCCACCCCGTGTTACTAGAAGCTATTCTTATCTCATTGTTTTTAATAATTTTCAAGGGAGTGCTCACA
CTTGGGTCTTCTCTGGTGGACACAGAGAGTTCATGTGCACTGAGGTTTGCTGCCACCTCTTGCTGCACCGTTGACTTCTATTCTGGCCAAATTC
CTGGACTCCGGGTCACACTAAAGCCACCTGATTCCTCTGATTCATCGCAATTCCACAGAAACACGTATCTCTTCAGAATGATTCGGCGGGTCTT
TGGAAGGCGGTGTGAGGGAGGCTGAGAAGTGCTCAGGACGCCCTGAGATCTGTGAGCCTTGGTTTTTCCTCCTGTGTAGTGGGCATGGTCGTGC
CTTTCACTCAGGCCTCACAGGGTTGTTGTTAATAATAATAGCAATAATAATAATAATAATAATAATAATAATAATAGCAAGATGGTGTACTTAT
GAGGAGACTTAGGAGACGAGGCATCGCCGCTCTTTCTACTGAAGGGTGGGATTGTTGGAAGAGAAGGGAGGAGATGACAGGAAGTTAGGGAACG
AGGTGCCCCTCCTACTGCCTCTCATCCACGACAGGGGACTTTTATCCCTTTGTTCTGCTAAGGACCTGATAAAACGGAGGCGTCACAATTACTC
ATAAATCAATGCATGCATAACAGTAATAAACAACTAATCATTTTCAGGGCCCAGTCTGGGCCCTGTGTGTTGTGCATATGCAACCTACCTCCCT
TCCCTCTGTCTCTTTGCTTACCCCGTCTCCTAAAGGCATTTAGGGAGAGCATTTCATTCATTCATTCATTCATTCATTCACTCATTCATTCTTC
TCTTTTGCTTTTTTGGAAACAGAGTGAACACTGTTCAGCCCAGGCTGGCCTGGAATTCACAGTGATCCTCCTGCCTCAGCTTGCCATGTCCCCG
TTCATCTATTTCTTCATTCATCATCGCACATCTGCTGTGCACCTATTGTGTCGGCTAGCACTAGACGACCGTGAGTCCGCTGAAGAGGCGTTCT
CAAGGTCAAGGGTTCATGAGGGGCGACAGTGAAGCAGGCCTTCACTGTTCTGTGTAGACGTCTCTGTGACAAAGGACAGGTGCAACGGGGCCAA
ATACAATTGTGTGTAACCTAGCGAAGGGCAGATGGTTTCCCCGGGCAACTCGTCTCTCTCTCTTCAAACCTATGCCAGGCAACAGAGTGTGCAT
TTGGCGGCGGTGTGGGGGCGCGACTTGTCTGTCGAGGAACCAAGACATACAAGGGTGGCCTGCTCTGGAGGCCTTGGCATACAGGGGGCTGAGG
ATGCTTTAAGGGCCTTGACATCCATCCTCGGCACTAGGTCTGATGCCAGTGACCTGGCaAGCAGCCAACAGATCCTTGGCAGAGACGTACATCG
ACACACAGGTGTGGTGCACAAACTTAGACTGCAGTGTGCAGAGTAGGGAACTTGTTTGGGGGTTCTGGGGTGCTCAGAGACCAGTCTTACTAGT
GTCTCCTCTCAGGCAGCCTCAGATCTGTGCATGGAGAGCTCGCAAATAGCCCCATTTAGCATTAGCGCCCACCTCAGGCTGGTGTCCAGCTTTC
CTCTCTTCCATTAGCATCCAGTCCACCTGCCCTGCCTCCTTTCAGAGCAGCAAACGTTCAAGAAGATGCTGAGCAGGAACTCCACACCCCAACA
CTCCCCTCCCTCCTCAATGCCCACACCCATACCCAGACTCCCTGCTGCTCCTGCCCAGGTTGCTGTGGGCATGATGCTCTAGGCCACAGGTACA
GGCGGGGGATTAGTCAGGCAGCCTTGGGAAGCCACCTTAGGCTTTTTCCCTGCATGGCCCCTACACAAGGCAGATAGACCAGCACAGCCTGGGC
AATCTGGGCATCTATCAACGAAAGAAGTCGCTGAGTGGAAGGACAGAAGAGCACGCGGCCTTAATCTAAAGAGACTCTTGTGCAAGTCTGGGCT
AAAAACAGGCTAGTCATCTGACATAATGTGGGGCTAGGATTCCAAGGGAGTAAGACAGGACAGAACATGCAGCGTGAAGAAAGAAAGCAGAGTC
CCTGGGACTCTCTTGGTGCTTTGGTGGGGTCGGCAAGCGATATAAGTGTCTGATCCAATTTTAGGATGTTTCCCAAGATGGCAGAATCTATAGA
CATCTCCTTACCTTATTAAAATGGGCACAGGTATAGCATTTGTGTGCAATCCCCTCTCTCTCCTCTTCTGTATCTAGTTTGTGTCATCTCACAA
TGACTTTACCCTACCTGTCGCACAGCAGATGCCGTATCTCTGTCCTGTATGTTTGAGCGGTGGTGGGCCGGACCCTCAGAGACAGAGACAGGAC
CCACTGCGTTCATTTTTTCCCTAAGGTTGTTTTCAACCCAGCACATCAAGTACAGCCATGCTTCCCACGCACGGGCAGCCATCGCTGAAACTCG
GCAGAACTCGTTGATTCACTTTTTCAAGCTTGTATGTGTGTGTGCACATGTGTGTGCAGGTGTGTATGTGGAAGCAGCAGCTGCTGTCCACACT
CTTTCATCACTTGGGATTTTTTTTTTTTTTGAGGAAAGGTCTCTGACTGAACCTGGAGTTCACCATTTCAGTTGGCAGCCCCCCTCCCCCAAAT
CCATTTGTATCAACCCCCAGGAATAGGGTATAGGTGCCTACTGCCCACTGGCATTTGACACAAGTGCTGGGCGTCCAAACACACGTCTTCATGC
TAGTCCAGCAAGCACCTTATCTACTGAGCATCTCCCAGCCCATCAGCCCTCCTTTTTTTGTTTTGTTTTTGTTGTTGTTGTTGGGTTTTTTTTT
TTTTTTTGGTTTTTTGTTTTTTTATTTTGTTTGGGTTTGGTTTTTGGAGCTTTCTCTGTGTAACATTGGCTGCCCTGGAACTCACTCTGTAGAC
CAGGCTGACCTCAAACTCACAGAGATCTGCTTGCCTCTGCCTCCCTAGTGCTGGGATTAAAGTGGGCTATAAGCGCCCTGCTCTATTTAATTTA
AATAACATTAGAACAAAAGACACAAATAGGGTCCAAGCCCAGCCCCTGAGCTCTACTGAGTCACTTCCAGAGAACTCCAGAGAATGTCACACAT
GGCTTTCCCATTCTCCAAGTGGATGGCTCACTCTCAAACTGCTGGTCACTCCGCACAACCAGGCAAAGGTTGTTTCTATGGAGAACTGAGTTCT
GTGCTTCAAGTTTTCAGTTAGGCTCTTGAGGCTTGGACGTTGGCCAAGAGCAGAGACTCTGGTCTGCATTCTGCCTACATGGACACAGACAGGA
CAGGGTCAGGCCAGCTGGACACAGGGCCTCTTCGCACGAATATCAGTCAGCCTACAGAAGCTGTGCAAGTCCAATGGGTTGTGTCCTGCCTATA
AGTCCCTAAGTAAATTATGCAACTCTACCGTCAACAGATCATGGTGGAAATAGCAAGAGGAAGGGAAATGGAACGGACACACGTGTAAGTGCCG
GCTGTTAGGAAGCACCGGTGGGAAAATAAGGGGGGAAATGCTAAGTGCATGCTGGGAATATCCTCCAAACACAGAAGTTATCCCTGGAATCTCG
GGGAGAGCATTCATTTCCTTGGAAAAAAAAAAGTCCCCAACCGTGTGTGTGTGTGTGTGTCTGTGTCTGTGTGTGTGTGTGGTTAGGCTTGCAC
ATGTGTACACATGTGTGGATCTCACAGGACAATTTGTGGGAACCAGTCTCCCCTTTTTCAGTATAGGTGCCAGGGACTGAACTCAGGTGGCTGG
GCTTGGCCGCAGGCCTCCACTCACTGAGCCCTGTCGCTGGCCTGGGTGCGTGCATTTCCATGCTCAGAGTTACTTGTACCCAGAATTGGACATG
GCATAATCTACGGTCCCCTGTAACCTTCGAGAATCTTCGGGGATTTCCTGAGTTTGTGAAGAGCAGACCTCCCTCCCACCCCTTCCTGGCTGTT
TTGATTTTTCAAAGGCAACATTTTAAAAAGAAAGTTTCCTTCCACTTGCTTTGGGGTGCCTAAAGTAGCAACTGTGCAGAAGGTTCCCTCAGAG
AGACACAAAGAGAGGGATGTTACCGQTACTGCTTAAGGAAAATGGCTACCCCACATCCCCCGGGCAGAAGATCCTTTGCATGTGTAATCTTCCT
TTTCGTGACAAAGGGCAAGTTTCACATCCCCAGAAACAGTCCCCAAAAGGAGAAGATGACTCATGTGTCCCAACCTACACCAAGGAGAATGAGA
GTTACGGAGCCACTGAGAACTGGGGGTGGGGGGTGGGGATAGAGCAGCCCACCCACAGGCCAAGAAGGGCAGCGGGCTCCCAGCAAGAGACTGG
AGGCCAACGGGCTTGGAGGGCCGCCCAGGGGTCAAGCTGGAATGTAGTGATGTTTAGAGCCTGGGCAAGCAGTGATCTTCAGCCACCTATCTTC
TTGCTTACTTTTTGCTTCCTGCCCTCAGCCCTCCTAGGGGAAGCCATATACAGTGTTATAGTATCTTGATCCTGCAGTTTAGTCATTTGCTACA
TGCAAAGGGAAGCAGTGTGTCTCAGGCTAGACAGAGAGGCAGGGTCCTGCTGATTCTGTATTAGACCCCAGGGCTGTCAGTGTCCACCACTCAG
TCCTCCCCTGAGGCCCTGGGACTGCCTGGCCTTGCAAAGCATCCCCCACAGCCCCAGCAAGTGTCCAGGAAGGCTCACCCCGTGACCTTCTTTC
CAACTAAATAAAGCAGTTCGTATTTTGTTCTGCCCTTTGGATCGGCAGACTCGCGTCGAGCAGGACGCCACATGTTGCTGTCTTGGCTTGTTTA
TATAGTGGATCAGCCCCTCGCTGTCATGCCTTAAAGCGTGGCCAGCTGGTGATGCAGCCATGAACCGCCAAGTTCAAGTAGAAATCAGTCTGAG
CAGCAATTCTGACACCTGTTGAGCGTTCCTCCTCTTCCCCAATTCCTCCCCCATCAGGTGTCCTTGGGCAGCTCACAAAAGCTGTTACCACTTT
GTATTTTGCAGAAGAGAAAGCTACGGCTGTGAGACAGTAACAAAGTTGCCCTTGAGTATGACTCTACAGGCAACGAGTGGGATTCGAACACGGG
TATACTGGGTTGCTGAGCTCTCACACTTAGCCCTCTCCCTATGTACTTCCAGCTCTGGAATGTTCTGAGCAAGCTTTGTCTCTGGATGCTTGGC
TCAGTCCAGACTTCCCCCTTCTTCCCCTGGTCTTTCTTTTTTGATGCTCCTTCCTCTTGCCTTCAGCCCTCTTGTCACTCCTGTCAAATATCAG
TCCTCTGGGATCACAGCCATCCACTGAAGCTGATGCGGCGGGTTCCTTCAGTACCAGGAGCAGAATAAAGATTTCGAGGCTTCCCCCTCTCTGT
CCGCCCTCTCACTGGCTCGATGATTCAGACCATCAGTGCCTTACCCCTTGCCCCTCTACCACCAAAAGCCTCTGCCTCCACCCAATGCCCCTGA
TCAGCACCAAAGTGATCTATGTCCTGGCCAGCCAGGGTTTCTGGGAAAGTGGGGTCTCCCATCAGGCAACAGACTAAAACATGAAGTCTTGGAC
AGCCCACTCTCTCTGCCCTGCCTTAGACCCTGAAGATCCCAGGAAAGCGAGTGTTATTTTGCTGACCCGTACTCCTCTAGGTCGGTCCCTTGTG
AGTCTCTCCTCTCCTGGCGGCTGCAAGTTACGGCGGAGAAAAACACTTGAGAGTGTTTCACTCTCCACGCCCATGTACTTAAAATACCAAGCCC
ATAGGTCATTTTAATAAGGGAATTTGGCTAACAACACATGTGCGCTGGAGGCCCTAGCTCCCACGTCTGCACGTGCTGGCCAACCACGGCTGTG
TCTGAGCAACCACGGGCCTGGGTGGGTGGAGAGCAGAGCTTGCAGCTTCTGTAATTCTGAGCACCGAGATCTGCCTCTGGCTACCTGGAAGCCG
TTGTCAGGGACGGGCCTAAACCCAGCTGGAAAAATACAAACAAGAATGGAGGTCAGGTTAGAACCAGGGTGACAAAAAAGTCACCATGGTCTAT
AGATCATAAGCAGATGTCCTTGTACTCAAAGCTCTTACCAGGACTGGGGAGGTGGCTCAGTTGGCAGAATCCTGGCAGCACAAGCAGGAGGTCC
CGAGTTCTATCCCTAGAGCCCAGGTTTTAAATTTTTAAATTTGTAATGCATGTTTGAAATCCCAGTTCTGGGGAAGTGGAGACAGACACATCCC
TGGGGCTCACTGCTCAGTGGCCTAGCTGAATCCATGAGTTCCAGACCAGTGAGAGATCCTGTCTCAAATCACAAGATGGGCAGAGCTTGAGGAA
TGATGTCTAAGGCTGACCTGTCGCCTCCACATAAAACTGCACATGTGTGTTTGTGCACACACATACACACACACATGCACACACGTCAGCTGCA
ACCCCAGCCCATCGTGGGGATTGCCACCAGTTTATCTCCTCCCAGGTGAACCCCATTACAGACATGCAGTCTCCCTAATCTCCCTCCCTGGTGG
GAATCACTAAGTGGAATTCCTCGTATTGACACATTAAAGGATGACCATAAAGTCATAGGAACCATGGTGCTGAGCAGTGGCCATACCAGTTGGG
GGTCACAGCTGTATTTCTTTCCTAAGTCAGCTAGTCTCTGGCTAAGCCGACACTCTCACAGTCTTACACCTTCTCCAACCGCCAGTCACCTCCA
GGAGAGACTGCCGCTGAGTCCTGAACTTGTTCGTAGGCAGCTGCCTAGAACAGCTTGCACATTACAGTCACAGGGGCAATTTTCACTTTGGAGG
ATGGTAGTTGGGCAGTGGGTGGGAGATGGGGTGAGTCGAGGCTCCTGAGTTTAGATTTTGCACAGGTGGCCATTTATACGCCAATGGTGGAAGC
ACAAAGGTTGTGAGTGTAAAGTTTAGGGTCATTCCCTCTGTGGTTAAGTGAGGTGGTTAATATTTTATGTCTGTCTGTCTGTCTGTCTGTATTA
TGTATTTGTGTGTGTGTGTGTTGTGGCTTATCTATGGAGGTTCAAGGATAACTTGTGTGAGTCAGTTCTGTCCTTCCACTATTTGGATCCCAAG
AGTGAAACTTGGGTTGTCCCGTTTGGCAGTAAGTCCCTCTGCCCACTGAGCCATCTTGCCAACCTGACCTaAGTGATTTTTCAGCATCCAAAGT
CACCTGTTTAAGTCTTTGCCTTACACTCTTTTAAGAACTGGTAAAAATCCATCTTGCTCATTTCACGGGTCTTCTCTGACAACCAAATGAAGTA
ACTTCCATCGGAATGTGTCGGAAGCATTGGAAGCTCTGCATACACCTAAACATGTCTACATTGTGAAAACGAGATTCCTCATTGTAGGCAGAGT
TATCAAAGGAGCCCTGGTGAGAATTGAGCTGTTCTAGAGTCCAGAAACCCTTGCAATGCTAAGCCCAGGATGCTCTGTATTTGTGCCAGGATGA
AGAATGTGCCTGGATGGTCTCCACCCACCCAGGATCTCTGAACATGTTCTTATTTAGAAAATTCCCCGATGCTGTGAGGTTAAGGCCATCCATG
AAGACCTTCATGTGGAGACCATATGTGTTAGCCTTCATGTTGAGCTCACCTATGATGGCCTTCATGTGGAGCTCATGCATGACAGCTTCTAAGG
ATGGGGAGATTCAGAGACAAGGGCATATAAGGAGGCAGCATACAAAGGTAGAACCAGTAATTGGGGCACTGAGACCACAGCGCCTCCAGAAGGT
CAATACTCATGACAATCTTAGGAAGTCAGGAGAAGGAGAAGGAAGGAGCCAACCACCCCCATTGCCACCTAAGGTGTCTTACACGCACCTTCAT
CTTGGCCTTGTGCCCTCCAGAACAAGAGACTGAGTCTGTGTGCATTAAGCCATAGCACTGTATTACATCAGCCCCAACATTGCTTGAACTCACT
GTTCTTGAAGTCTTTGCACTTCCGAGGCTGAAAGCAACAGATGACATGATTCTGTGTGTGGTGACAGTGTGCTAGATAGTCACAGGTTTGGAAG
ACATCATTCGAGACCTTCATGACGCATTTCAGGCAAGCCATGGCTGAAGCCAGCATTGGAGAATGTTAGATGCTACAGTACTCCCCAACCTTGA
GCTGTTACCGAGCTACAAGCTCCCATGTAGGCATCTTACTGCCTCGGGCTCAGAAGACACTATCAGAAAAGATCTGTGAGCTATTCCACAAGTA
GCTACTCTTGACCTGGGACCCAGATTGGCAAAAATGTGCGACAATCCAACTCTTCGAATGCTCTATATAGAAGCACCTGATCTTATTGTCTGTT
CACTCTCCTCACACTCGCGCATGTACACACACACATACACACACACACATACACACACACACATACACACACACACACACACACACACCAAGGG
CCAGCTCAGGACCAAGCTATGTACCTATCAGTCACTGGTGTCTTCAAGGTCCTTTACCTATTTCCCCTTTGGCTTTACAATCTTCCATTGCCTG
AGAGCCTCTTCCTGTTCTCCTTTCTCACTTCTGAGGAATGCGAGGCTTTGGAGAAATGGGCCCTCCCCCCACCCTCACCCCCACCCCCAGGCAG
ATTAAGCTTGGACCATCACAAATGCTGTCAAAGCAGAAGGCCTAATTAATCAAGGTGAAAAGTGATTTATGGGGCCAATCGGAACTAGTCACAA
GCAGACTCTTCCTTCGGGGAAGGTCACCTGGGAAGCAGGTCAGTGGGAGACTCACTGGGCCTTGAAGTTGGCAAGACTTGAGAGAGTGAATCTG
GGTGGCTGGAGAAGTGGGGTTGAGTACAGAAGTCCCAGAGTCTGGAGGCACTACTTAAAGACCTGAGCTCATGAGCTGCAGAAAGAGGGACCGG
TTCTAGGGAGAAGATTCTGGAAACTGGCTCTAGGATTCCATGTTGATGATACCAAGGTCTCGCCCGTCCATCATGCGTCCTTGGTGAGAAAGGA
AGACCCATTGTGAGGTGTGCAGGGTGCTTACCGTACAGCAGCAGGCTCGGCACTGCCTTTACACAGTCAAAGGCTACAGAGAACGATGAGCAAC
TTCCTCACGTTACACATCAGCACACCATTCTTAACATGCCCACCCTCAGCCTCTGTGGACCATCCCTCCAAGTCTGCTAGATGCTAAGGTAGAA
AACCATATGGAATATCCCCTGATGGTGTATCCGCAGGTGTCTGATGCAAGTGCCAGAGCCTGGCAGCCTTCTTCCTGGGGAAGCTGGGGAAGGA
TTCAGGTACAAAGTCTCTTGGGCCGTAGAACCCCAGTCTCCACAAACCACTCCTGTCTGGTCCACTGCGCCTCTCAAGATGCACAAGACGCCTC
CTCCTGAACCTTGCCTGACACTATTGTTCTCTTTGGCACACACTACCAGACAGACTTCAGAGGCTCTGGGGTCTTTGGGAAACTGTACTCTTGA
TACGGAGTGCTGCTTGCTCTGGTTATAGGTTGTATTCTTGGGAAATGTCTCAGTGGCCTTCCACTTTCAGCTGGAAGTGGTATGGTTTTTCTAA
GGAGGTTAGGCGGGTAAGCACGGTTTTGCAGTAGCCATTGCCTTTTGGACACAGTTTGTTCCCATTTCCCTTGAGTTTCATTGTGTACTACGTA
ACAGCAGAGATTGTACTAGCTAAGGAACTAATATTTAGGTAGAAGACTTAATATTGCACCCGGCCAAATACGGGCATTCCATACAGAGCTCTGA
GACGGGGAGAGGTGCGTGCGTGCATGTGAGCCTTCGGACCCCCATCGGCAGACACCATTACAATGCTTTACCTCATTTATCTTTCTCTCGGGCC
CAAAAGGTAGAAATTTCCGAAAATTGCCTATACCAATTTTCAGGCAAGAACACCGAGCCTGAACAAGAACACAGAGAAGTCTAGATCTAGTGGC
TTGTCTGTCAAGCATCTGGAATTTAAACCACAATCCACCCCCTGAAGCCTGCTCTGTCTGTCCTGTCTATGTCCCTTGCTCTGTCCTATCCCAG
CCATGCTTTCGTAGCATCTAGTCATTGGGTATTTATAAATACTATTTACCATCAAGCATCAATTTCTAATAGGTACAGAGAATAACATCTAATT
TAGCATGTGCTGTGTTAATAAAAAGATAGAGAATAAGAATATGTTCTATGGAGGTGTGGTCAGGTATGGGGGTAGAGGCATCCCCTCCCCCTGA
AGTACCAGCCACATGACGGTATAACATAGAATAGACTTTATTCAGGCCATGGGAAGGGGAGTTAAGAGGGTAGCAGAGACAGAAACGCAGAGAG
AGGGAGAGAGTAGAGAAGTAGAGGCTGACCATGACCACGTGGAAAGAGGGGGCAGGGAAAGGGGAAGCAGGGGCGAACGGAAGAGCCCAAGAGG
GCAAAAGAGAAGCAAGGGAGCATGAGAACAAGAGAGAGAGGAGGGGACAAGCAACCCCTTTTATAGTGGGTCAGGCCTACCTGGCTATTGTCAG
GTAACTGTGGGGTGGAGTTTAGACAGAATGCTAACAGCATGTTTTCACACTTACAAACTCAGTGTTTGTTGATTTGGTTGGCCTGCTCATTTGT
TTTGAGACAGAGTCTCATGCAGCCGAGGCTAACCTCACACTCACTGAGTAGCTGAGGGTGACTTTGAACTTTTGATCCTCCTGCCTCCGTTACC
TGAGTGCTGGGATTACAGGTGTGCAGCCTGGCACGCAGTGTATACAATGCTGGCATAGATCCTTGGGCTTCATGCTTGCTAGACCACCACCCTA
ACCGCTGACCTACCCCTGGCCCCAGAAAGTCAGTCTTCTCTAATCCTCGGAAGTACAGAGTAAAGAGAGAAATGTGTGACGGTGTCTTTTCTTA
TTCTTTTGCCTGGATTTTACAAATATGCAGGGAAGACAGAGGGAGGTTAGCAGTAGATAGGTATGAGTCCCAGCTGTCAATTCCCAAAGTCCTG
ATATGGGAGTAAATTCTGATCTCCTGGATCTTATTGGTAATTAATGATTGACTTATACCTATGTGACTATGCACTATGCCCCACCTGGTGAACC
CTCTGTGTGCATTACAGATCAGAAATAACTGGTGAACCCACTGTGTGCATTACATGGTGAACCCACTGTGTGCATTCCAGATTGGAAACAAAAC
CTAGGTGTACCAGGAGAACAAGTCCTCCTGCCCCAGCTCTGTTGCCTTTCAAGCTTGAGGGCTGGACAGCATAGACTGACATAAGGTAGAGAGG
CTCAGACCACCCCTCAATATGTTCTGTTCTGTTTCCTGTCTCCACAGGCAAGAGCTTCACTCTGACCATCACCGTCTTTACATATCCCCCACAA
GTTGCCACCTACCATAGAGCCATCAAAATCACAGTCGACGGCCCCCGAGAACCCCGAAGTAAGTGAGCCCCCTTGGGGCTGGTAACTGTCTCGG
CTGGTGTACACAAAGACCCACCAGGTCTCACAATGTATCTGTGGCTTCAGCCTCTTTCCTGAAGAGATGTCAGGCCCCTGAAGACAGGAGAAGT
TTCCAGGTCAACTCTCTCACCTCTCAAACGTTGTTTGTGTGAACTGGGTTCTTTCTAAGGCTTTGGCTTACACGTCAAAGGGAAATTAGGCCAA
CTCAGATGAGTTAAGAACACTTTGGGTTTGTTTTTGTTTTCTGAGGCAAGGAGTAGACCCCCACATATACAGCAATGTTCAGTGTGCTCAGACA
CAAGCAGTGGATAGACAATTGGCCAAGTACAGGGTGTGACAATGAAGACCTGAAGGTCACGGTGGACAAGAAGAGATGGCTTACCATCCATGCT
TGTGTTAATTTGTTAAGGCCAGAGATGAAATCTGTGAAAATCATCCTCTCTTTTTCCCTTGAAGGGATCTTATTCTAGGATTGTTTAAAATAGA
GCCTGGGCCGGGCGAAGAGTACTGGGGGAAACCAGTAAATATAGAAAGATAGCCTCCAAAATACCCCGAAACTCCTGGGAGGAGAGTGTTTAAA
AAGGAGTGGGGTTGTTTAGTCTGAAGGAAAGATGTCTTCAGAGTGACTTAAGGACTATCTTGGAGTAAACACACGAGTGACACTCTTACAGGGC
CACACCCCGGCCCAGGAGCTGCAGCTGGGCCACAGTCCAATTCTGTAGGCAGATGAGCCCGGAACGAAGAGCAGCTCACCTCCAAACCATCTCA
GGGGCCCCTGGGAAGTGATACCCTCAGAGGCTGGGATGCGAAAACATGGGGTCCCTCCTGCACCCAGAAGCTATGGTGCAGAGAGATTATTTGA
TAAGAATGAAAGGTACTGAGAACAGGGGCCCTAGAACAGTCTCAGAGCCTCCCAGAACCCCAGTTTCTTGTAGAAATGTATGTTAAGGTGACAG
CCCCATTGTGAAACTTAACTAGATGAAGCCAGGTGCCCGGTGCAGGGCATGCGCTTACAGAATGTCAGGGTCTCCGTCGGACCTTCCTTGCCTC
GACCCCTCGTTTCAGTGTGGTGGTTCCATTGAGCCTGCTGAGGTAGTTCCCTCAACAGGCAGCCCTGTCCACACACGCATCTCATCTGGGCTCT
GAGGTAGTAGCCCTTGGGAATTTGGCTCTCTGAGGCTATTCAGTGGTCACTCTGGCTATGGCAGCGTCTCCTGCTTTCATGGTCATGACTTCTG
GTATCTGGACCACCCTTGGAACCAGTCACCTCTCTGCCTCCATTTCCCCAATTTTCCCTGAACCAGTAATACACAGAGAACAGCTGCAGTCACC
AGCAATACCACAGGGCAGAGTCTTCACCCTGGCTGTGCTTCCCTCCCCTCCCTGGGCTGGGCAGCTCCCTCCACTCTGCCCACCCTGTTCCCAT
TTTTATTCCCTACCAAAACCTGTTGAGCTTTTCCTTGTTAACGTCCTTGAGATTTCCTAGCTGTGGTCAGGTTCAGGGAACTCTGGGGCTTGTG
CTGGATACCATGCCCTACTCCTTTAGAGTGTCAACAGCAGACCAACGTAGCACACAGTCCTCACACACCCCAGGAAACCTCTTGCCACTCCCTG
GTTTCCCAGAGTAGACAGTCATGACAGAACACCCGAGGGCCATGGCCCAGTGCAGGGGACTGGAGAGGAAGCAAGCCGAGGTGATGACTTAGCA
CACGGCCAGGAGAGGGGGCGAGGTGTACGAAGACTGATGCTGAGACTGACCGTGCTCATAGGATCCCTCCTTTAGGTGTCCGTGGCTCTCTAAA
GCAGCTGCTGCCTCCAGGACAGATTATACAATGGCTGTGCACGAGGAAACACCTCAAAATCGCCACCCATCAGAACCCTTCTATTTTCCTTATT
CTTCCACACCTAAGAGAGACAGGCTTTCACCTTGTGTGTATTTTTTTTCCTGGAACATTCCAGAGAAGTGAGGGGGGAGCTGAGCCTTTGAAAA
AGCAAACGCCACCGGAAAGGGAAACGGCCCCCATATTGGGGCCCCTGCTTGCCTGATGTGTATAGATCAACTACTCAGATAAAGCACTTGCCGC
CTCTGCTATTTTATAACTTGTAAGAACAGTGGACCATTAGTGCTTAATTGTACTATTATCCTCCTATTCTTTTTTATTTATTTTTCTGCTTTGA
GTTGAGAGACAGGCTCTCACTATGCTGTCCCGGTCGGCCGGAACTTGCTATGTAAGCCAAGCTGACTTTGAACTCCTGCCTCCCCAGACCCTGA
GTGCTGGGCTTACACCGTGTGCCACCACATCTCATACCGGTTCTCATACCACACGGATCATTTCCACTCGCACTCTGCCCATCACCACTTTTGG
ATGCAGTGAGAAGTGAGGACCCCGTGTGCCCGTCAGCGTGGCCAGACTGGCACACCCGTGGAGATTTACGAGCGCTGTCCCACCCCTCATTAAG
GAAGGATCATAGATTCTGGATGAGTCTGGCCCAAGAACCCTAGTGTTTGCCACACTGAGCAACATCGCTCAGTTCTCCCACTTTACCAAATCAA
AGACTCAGAGAGAAAGAACCTCTCTGATCCTTCCAAGAAAAGGAAAATCAGCCCTAGAGAAGGGAAGCAACCTTACGTTTAAACAAGATGATGA
AGCCACAGGGTGGAGGGGGAGTTTTTCACTGTCTGACTTTGGTGTCTGACTTTGGAACACACATCTGTCCATCTGTCCATCCATACCATGCTGT
CTGCACTTCACATCCTATTAGTTCTCTCTCCTGCCTTTGCAATTTTTGTGTTTAATTGTGGACTGGCTTTCATGATTTCTCATGTCGGACCCAC
CTCTGAGGCTGCCCCATAGGGCACAAGGTAACCTCGACGCTGACATGGTTGTCAGGACTGAAGCTGTATCGTAGTAGGTTGTGTGCTCCAGGGA
CTCCCCAGCCTTTTGCAGTTCAGATGGTTACTGGCCGGTCCCAGAAACCCTCTGCCCCCGAGTAAGTCAAAACTTGCTTTTCACACAGTCCATG
GGATCCTTCTACATCTCCAGCCAATACAGAAATCTGTGAGCTTCGGGGAATTCAGTTCCTTACAGGGGAAGAGTGCCACCGGAAGGTTTTTCTT
TTCAGGCAGTAAATAGACCATTTGATGACTTGTTAACATACTGGAAGATAGTGTGGTAGAACATATACTAAAAGGGTGCTGGTATATGTGTGTG
AGGGGTACATATGGGTGTGTGTGTAGTATAAGTGTGTGTGTTTATGGGTTCATGTCGAGGTCAGGGGTCAGACGTCAGCATCAACCATCATCGT
CTTCAACTCTACTTGTTCTTTAACACAGGGTCTCTCTGTGAACCTGTAGCTCATCTGCTGACCGATGCTAACTAGCCAGTAAGCTGCAAGGACC
TGTATGTGTCTATTCTCCAACACTGGGGTTAGTGCAGACATACCAAATCACCCAGCCAATTACACTCGAGGTAGGGATGGGGACATGAGTCCTC
ATACCTGGGTGGCAGACACTCTACTGACTGACCAATCTCCTTAGCACCACCCCCTGCTCCATAAAAGGGTTTTCTCTTCACAAACAGAATTTTA
AGAGTGGGTTTATGTTGCTTTGATATTTCTAGAAACATGTAAAACTCCATCTCTCAACCCACTCGTGATGAGTCTTGGGAAGTCTGGTACCTGT
GACTTGGGGAATAGACTTCATTAAAATTCAAAAAGGTCAAGAAACCAATGCTGGCTGTGTCTGCCACAGCTTCTGCCCTCTGGTAAAGCTGTCC
TTGGCAGTCACTGTTCAAGCTCTGAGATTCACAGTGACGAGAACAAGATAGACCTGTTCCTTAGAACTCAGAGCCATCAGGATGCAGGTCTCAG
TGTCTTAGTTGGGGTTTACTGCTGTGAACAGACACCATGACCAAAAGGACATTTAATACGGGCTGGCTTACACCTTAGGTCCAAGCCTCAAATC
CTTTGCATGACCCTTTCAGTCCTGCGCTGTCAACTGCAACTGAGGCTGCACCTTCACCAATGACCTAACATGCCCTCTCACAGTGCCAAACCTC
TGCTGCTCTTCATGCCCCCTTCATGCCTTCAACACCAATACCACCTGACTGACTCTTACACATTACCAAGTACCACTGCAGCATGAGGCACAAC
CTTGGCTATCTCTGGAACACAGCTTCTTTGTACTCTCAGAAAACACTTCTCAGAAGATTTCACCTCAGTGATGCTCGTCTCTTCTTAATCACTG
CTAATTTTGTAGCTCCAGCTCACGAGCATCCATTTTCCCACTACCCCCTTCTATTCTTGACTCTAAAGCTTCAGCCCAAACTGCTCAGTTCTGC
TGCTTGCTGGTGCTGGAACATGGCCCCCTTGTTCTATTACATTATCAGTGCTGGGATTAAAGGTGTGTACCACCACACCTGGACTTAAGCTTTT
CTTCACCAAGAGCTTGCTCTGGTCCCAGGCTGGCCTTGAACTCTGAGATCTTCTTGGCTTTATCTCCAAGGATAGCCCCATGCCTCGATCTAAA
TTTAGCTGAGTAGGATCTTCCCCCAAAGTCCTACTCCCTTAATCCGATATCTCCTACAACATGGGAGTTGGCTCCATTCTGCTTCCTGGTGCCC
CTTTACTATTTAAACCATATATTTTACATTTTTCCTTTCTAAGCTTGCTATGCTTGCTTAAAATGCTCTTCATGAGACTCAACCAGAGAACAAA
GTCTCTGCTCGGTGTTTCTGGACTTCCTTTGCCATTGCAATTAATCTGAGTCTCTTCTCCTTAGCCTCAGGCAGACTCTTCTGACAAGGGCTAA
AGCCACCCATATTCTTCATCAAAATACCATAAAAACAGCCTCCAGGCCATGTACTGAAATTCTTTTCCACTGAAACTTCTTGAGCCAGGTCTGC
ACAGTTCAAATCACTCTCAGCAAAGAAGTCTTCCATATTCCTAATAGGATGGCCCATTGAGCCCTAGTTAAAGCATTCCACTGCTTTCCAAATC
CAAAGTCCCAAAATCTACATTTTTTCCCTAACAAAAGTATGGTAAGGCCTATTACAGCAATACCCCATTCCCTCGTACCAACTTTTATCTTAGG
CTTTTACTGCTGTGAACAGACACCAAGACCAAGCCGGGTGTGGTGGCGCACGCCTTTAATCCCAGCACTTTCGAGGCAGAGGCAGGCAGATTTC
TGAGTTCGAGGCCAGCCTGGTCTACAAAGTGAGTTCCAGGACAGTCAGGGCTATACAGAGAAACCCTGTCTGGAAAAAAACAACAAAAGAGCAA
AACAAAACAAACAGACACCATGACCAAAGCAACTCTTATTAGGACAGCATTTAATTGGAACTGGCTTATAGGTTCAGACGCAGTCCATTATCAT
CAAGCCAGGAACATGGCAGCATCCACGCAGACATGGTGCAGGAGGAGCTGAGTTCTACATCTTCTTCTGAAGGCTATTAAGAGAATACTAGCTT
CCAGGCAGGTAGGATAAGGGTCTTAAAGCCTACACCCACAGTGACACACCTACTCTGACTGGGCCACACCTTCTAATAGTGCCACTCCCTGGGC
CAAATATATCCAAACCATCACATTCACCAACTGATCGTTTCTAGATGAAACTATCCTTAGAATGGATCAACTCAAGGGCCTGTCACCCAACTGC
TTTTTAGAGAGTTTTGTTGAAGTCGTCCTGGGAGTAAATGCACGTGCTTCTGTATGTGTGTGTGCATGCATGTACACATGTGTGACATGTGGGA
CACGCATGTTTATGCAGGTGTGCCTACAGTGTGTTCCCACGTGGAAGCTAGAGGTTCATGTCACGTGTCTTCGTCCTTCGCTCTCCTTCTGCCC
TTTCGTTTGTCTGAGTCAGGCTCTCTCACTGAACCTGGAACCTACTGAGCTTGAAATTCTCGCTAGATTGGCTCATAGTGAGTCTCCACGATTC
TTGTATGTTCACCTCACCAGTGCTAGGGTTATACGTATGCGCTGCCACACCCGCCTTTTAGACAGGGATGCTGAGGATCTAAATTCAGTTCCTC
ATACTTGTGCAACAAACACCTTACTGACTGAACTGTCTCCCAGCCTCATGAATTTATTTTCTTCTTTGATTTTCAAGGGACATAGCAGACAATA
AACAAACAAACAAACAAACACAAATAAATAAATAAAATCTTACATGTGTATGTATGCATGTATGTATATTAAACAGAACCTTTATATTTGATGA
ATAATTTCTAATGAAAAAGCAAAATGCATGAAAGACATCTGTAAACATTCGGTGATAATGGACTCAAAATCCAGACTGTTAGAAAAGAGTGAAC
TCGGTGTTTGTGGCTTTATTTCTTAACATTGAAAGCAGTGCAGATCTTTTTCAAGATATTCTTTTCTCTGGATAAGAAGAGTTACTAATAATAG
TGGCTGACATTATATAGCACTCATTATTTGCCCAGCACCTTTCAAAGTGCTGCAGGCATATTAATTTATAAAATCTTGGGAGAAACAGTTAAGG
TAGTTGAAGCAAAAGCTAATTAAGAACCCTACCCAAGGTCACCCAGCTAATCAGCCACAATGGAAGCTGTGGAATCCAGATGTTGCACACCTAG
GTCTGCTTCTGAGCACTAAGGTATTTGGGCCTCGCTCCATTGTGATGCTGTATCTTTGATAAGGAAGCCTTATCACTTGGGTGTGACTCACATG
TGGTTTTCCTATAGCTTTGAAGCTGCCCCAGACGTTTGACTGGAGTCAGTCTTCCTACCGGTGGGTGTACTGAGTTACCCACGTGCTTCCCAGG
GAGTTCCCAATCCTGCCTGCTGCCACGTGTGCTAACGATTGGCTCTGAGGGTCACTGAGGTCTGCTTGAGGACATGGTGGCAATGGGACCTAAA
CCAGGGATAATGCAAAAACCTATATGTTTCCCAAAGAGAAGAGTACGACAGTGACCGACTGGAGCAGCCCTGAGGGCAGGTGTCTAAACGGGGA
TCCCCTTTAGCAAAGGGTCCCAGGTAAAGCTTGGACACTCAGTGTGTGCTCTTGCGGTTGCCATGGCTAGTCATGGTGCTCACAGGGAACCGTG
CCACGTGCCTCCCAGGTCCTTGGAGTGTTCAGATATCAGGCGTGTCCATCAGTTCAAGGCAGAATCATGGTGCATAAAGGAAAATGGAGAGTTT
TTCCTTCCTTCCTTCCACCCAGATGGAAAGCAAAAGATAGAGCGTCAAGAGCAGTGCTCTGTATGCCTGCTCTTCCCCTCTGAAGGCATTTCAG
GTTAAGCAGAAGATTCTGGGCCGCTTCACCCAAGTATTTTAGTTCCCACTCTGCTAATCTCTCTAACTACCTAGACACTTAGGGAAGCAGTTCT
TAAAAGCATTAATAATAACTGTGCCTAAGTGTTTCTAAGGAATGTTAGAATCTCTTCCTGTTTATTATTTTAGTTATTGGACACTAGACCTACA
AGAGTTTTGTGAGCATCAAGATTTCCCTGAATTCAGGCTAAGGCCTAGAAGTTTCTCTGGGGCAACTGAGGATGTGGGAGAAAAGATCATACTT
CTGTCTTTGTGAACAGCAGCAGTCCTTCATGACACTACCCATGAACAGCAGTCCATCATGACACTGCCTGTGAACAACAGTCTATCATGACACT
ACCCATGAACAGCAGTCCATCATGACACTGCCTATGAACAAGAGTCTATCATGACACTACCCATGAACAGCAGTCCATCATGACACTGCCTGTG
AACAAGACTCTATCATGACACTACCCATGAACAGCACTCCATCATGACACTCCCTCTGAACAAGAGTCTATCATCACACTACCCATGAACAGCA
GTCCATCATGACACTGCCTGTGAACAACAGTCTATCATGACACTACCCATGAACAGCAGTCCATCATGACACTGCCTGTGAACAAGAGTCTATC
ATGACACTACCCATGAACAGCAGTCCATCATGACACTGCCTATGAACAAGTCTATCATGACACTACCCATGAACCCAGCAGTCCATCATGACAC
TACCCATGAACAGCAGTCCATCATGACACTACCCCATGAGCAACAGTTCACTATGATACTACCCATGAACAACAGCCTACCTTGATACTACCCC
AGTGAGTTTTTAGTCAGTGCCCCCCTTCTGTGCAGAAGTTGTTAAAGTTTAGGTGGCTCTTCCTGCCTGAAGCAGCTTCTTCCCAGGTACCAGG
AAGACTATACAGGCATAAGCAAGACACCTCCTCACTTTGGCGCCTGGAAAAGAGAGAAGAAACATATAATGAATGGAACCTAAACCATTTCTTT
GAATGTATTTCTTTCAGTTAGAGCACTGCAAAATCATATTGCTACTTACAATACCATCCCACTGAAACTGGCGTCTACGCTGATACACTGTCCT
TGTTAGGAATGATTGTACGAAGCGCAGAAACAGAGAAAAGAACCCAACAATGTAATACTGTCTCTGGTGAGGCTTCCGTTCTTAATCTTAAAAT
GTGTAAAAAGATTTCAGGATCCTTAACTCCGGGACCTAGGCTTTCACCCTGGCGCCATCGCGCCTGTGGGTCTGAACTTGCGCAATTATCTTAA
GTACTTTTTAGTCCTTGGTTTCCTCTTGGGTCAAACTAGGCAATGAGACGAGATAATCTCTAAATTAGTTCCCACTCGAGCTGGGGTAACAAAA
TCTTGTCAGCAAAACAGAGGTATAGCTTCTTCACAAACAGGGCCCCTTTACAGGCCACAACCCTGAGCTGAGGTCATCATCCTTCATGTCCTCA
ACCAGGGCTTCCACAGTGGTTTTCAGATTCTCCCTCTCCCTGTCCCTCTCTCTTCCCCTCCCTCCTCCTTCCCCTCTCCCTTTCTCTCTCCTCT
TTTTCCCTTCCCCTACCCTCTCTCTGTTCTTGAATGGGCTTTGTACTTCATCTTCAGTTTCTCTAGCTATAAGTAACAAGCCTAATGTCCGAAC
AGCCCTGATCTGATGCCTCTGCCACTCAGCTTGGGATGCTGCACGTCATGCTTGCCCTGCTTAGCTTTGGGATGCTGATACCTGGCTCTCTGTC
CAACAGAGACAATGGCCATCCATATCAGTGTCATGGGGTCCCTGGGAACCTTCCCTGGGACACAGTATACATTTAGAAGACGTCGGCTATTGTT
TGTATTTACCTTGGAGTCTTGTGGGAAATGAGAAAGGACTTTTGACAGAAACAACAAAGGGATCTGTGAATTCCTAAATTTAAAAATTCATGAA
GTGGCCTTAATGTTGCGTTTTTTTGAGATGTAATTAAACATTTATGCATTTTGATTTTTTTTATTTCAGATTTTTTTTTATTTAGGCCCAGTAA
ATTTCTTTTCTGGCATATTATGGCCCCTTAAAATATTCTGGGCACTGTTCCTAAGTCCCTCACGGTTCCAAAAGACTTGTCCCCAAGTGTCGTA
CTTTGTGAACAACTTGAAGAAAACTTATGTCCTGGTAACAAAGAAGCTCACACCTGACACTCGGGCCTCAGTGTATGCACGAGACAGCTGATGG
AGAGTCTTCACATTGTTCTAGAAAAGAACAGAATCCCCCCGCCCCCCATCTGCCCTTGCTGACCTTAGGGCATTTGTTCCAGGAACCAACACCT
CAGATATCTGCCACACCCAAGCCCCTCATATCAAATCGCAGATTATTTGTACATAACTTGTGAGCATCTTCCTGTAGACTGTCAGTCACCTCCG
GGTTACTGAAGTACTGGAGACAAGGCTGGATAAATGAATACCTGTGAGACTGTGTTGTTAGGGAAGTCCGTCTGTGCTCAGTGCGGCACTTTTT
CTTCCTCAAGTATTTTCAGCCTGGATTTGGTTGAATCCGGTTGTATCAGCTGCAGATGTGGAGGGATGAAAGATGACAGACAGACAGTACTGGG
ATCATCACTCCACCGTCCCTGTACACTCTAGCAAGACCTGTACGCGGTGCACACACACAAAGCACCTAGCCTAGACAGGTTTCTGAAGGGCTCC
CTCCCATTGTGTGTATGAGAGAGAGAGGAGGGGGCTCTCCTGCTACCTGTGGATGTTGCTCTGGGAGACTCAGCTGTTGTCCATCTTCCGTGGG
GACAAATGACCTCCCACCTAGCTGAAGGTCTGTGGCAGACAGCCCCTGTTGTCAGAGCACACTACCATGCCTACCTCTCCCCTTTCCCTTTCCG
AGGCAATTTCAATTCTTTCAAGGCCTAACCACTTTCCCTTTTCCGTTTTGAAGCAACTCCGGAGTTTTCCCGAGGGTTTAGGTTTTCCTGTAAA
GCTGTGTGCTGGGAAAGGCCATTAGGAAAAGCCTGGTACACAGTGTCTCAAAGGACCCGGCCACTGACACACACACACACACACACACACACAC
ACACACACACACACACACACAAGGATTTTTTTCCCCCAAAGCTTTGATCATATAAGTAAACTTCATGCTCCTCATTTTAGATCTGCGTCTAAGT
TCACTGTAGCTAATTTGAATCTGCCTCTTTTTTTCGTGTCTTGCGTCCGAAAACCTTTTGTAAGGTGTCCCGCCTTGGGGCTCTGGCAGCATTA
GATCGTGCCCTTAGGAAGGGGGTAAATTAACTCCAATCTACCTGTGAGTGTTTCTGCTGTCCTCAGACCCGGGAAAGCACCGGCTATTAGCCAG
GTGCCAAATGCCCTCTTAGTGGCTCAGTTTTCGTCAAGGTCAATCTTAAGGTTTTCCCTGGGAGAACTTGCAAGTGTATCAAACTTTCTCAGTC
TCTGTCCATAGTGAAAGCCAAGGTTAGAAAAAATATTTAAGCACCCAAAGGGAAGTCCTTTTCCTGTGGGTTCCCCCACACACACTGCCACCAA
CCCCCCGCCCCCACGCCACCCTTAAAGCAACACAGTGGAATTAATCTGCAACTCCCCAGGCCCTTGTGAAGTCCATTAATCGTGATGCATTAAT
TTTGTGCTCCTACCAATTCCATATGCCCAGGTTCCATTAAAGCAAGTATTGACTAAAACCCTCTCTGTTACTGAAAAAAAAGTCCAGCGTACTT
GCCTGCCCTGAAATGCCGAACAGTGGGCATTTTATTTGCTTGCTGGATCTTTTTACTTTTGTACGCCGTGGCTGGTAAGTCATATTCTAGTCAT
TTGAGGCAAAAATGTAAACATTTCAAATAAGTCACTTCGTAAGTTCAGGATGACTTAATGGTCCCTTTTGGAATTCTGACCCGAGTATCTCAGC
CTTACTTGCCTGTGGGGTGGCTTCACGGCGTTTCTGGTCCACTCATTTTATAATGCTACCAGCAAACGTACAGGAGGGACATTTTTACTCCTGA
TCTGACCTTGTTCCTGGGGCCCCAGTCCCAAATTTCAGAAAAGAATCAGACGGTTCGACAGGGAGTAAGAGAGGGAATTGGAGTCAGACCTCTG
ATACATACTGGCTGTGTGCCTGTTTTTCCCTCTGTAGAAGGGGTTTCCAGATGCCCACCTTCCAAAGGGGAAGATAGACCAGGTGGGCGCCATT
GCTCTGAGGCTAGCTCCCTGACTCCAGCTCCCGAACTGTCTTGGCTCTGATAACAAACCTTCCTCACAGGGATTGCAAGACCATGCATGGAGAC
TTCCATCTGAGGCTGCTTCACATAGCCCTGCACATAGCACCCTGTAACATTTAAAGCACAAACCACCAACACAGGCAAGTCAGTCTCCTTCATC
CCCAACCTTGTATTTACCAAAGGAATCACATGGAATTCTGCCGAAGTGTTCACCAAATATTTACCAATACCAAGTTATTTAAAGATGAGGGGGA
AACCTGGCAAATTGGTTCCACAGGACTCAGCACTTCGAGCCTTTTCTCTTTCAACTTGACCTCACGGTCACTTTAAATCCCGAAGCACCAGAAA
CCAAACCCAAGCCTTACCAAGGCACCACCTTGTGAAACCTGCATCGCGCCTGCCTCTTCAGAGAACAGGCAGGTGCGTCCAGATAGAGGCTTTA
TCTGTCATATGTAGCTCTGTTTCCCAGCAGAACATTGGGCATCCATGTTGCTGTCACCTACCAAACCTCCTACCATCTACAAAATTCTACTGGG
TCAGCTATGAGTACCATGGGAGGGGAGTCTCTCTACTGTATAGGCTTTCGTCTTATTTGAGTGGTATATATCAAAGTGTGGCTTATCAACTAAG
TAAGAGAAATAGCCTATCCACTAAGTAATAGGAATACAATAGATGTGAATGAGATATTTGCAAACAAACCAACCTCAAAACCGACTGCTAATGT
TTTACAGGTATTGGGCAAGATCCTTAGTGATCTTAAGGAAAATAATGATTGTTCTCTTAAAGATAATTTTGGTTTTGTTAGACTTGCTTGTTTT
ATATTTTGTGCATGAGTGTTTTGCCTGCTTGTATGTTTGTGTACCACGTGCATGCCTGGTGCCCTGGACGCCAGAAGAGGTCCTTCCATCCCTT
CAAACTGGAATTACAGTTGGTTCTGAGCTGCCATGTCGGTGCTGGGAACCAAACTCTGGTCTTCTAGAAGAGCAACACGTGCTCTTAACTGCAG
AACTATCTCTCTGCCTGAAAGATAGGTTCCGTCTCGTCTCGTCAGATTGGTGAGAATGCAGGAGAAGTCCGCATTTAAATTAGCCACCCCTGTT
AAGCTGGACTTGGAGAGGCTTTTCCCTCCTCGTTTGCCTCCCTCCACAAACTTTTTAAGGCACTTGGAAACAAAGGAAGCAGCCTTTATCCACA
GTTCAGAGTTTTGCTTAAATTCATTTTCTTTTCCAGGACCAAATAGTTTTTCTTCTGCATACGGCTCTCATGGCGGCAAAACCATAGGAGAGAA
CAGCGTAGCCTGCTGCTTCTGAGTCTATATTTCCTGTGTGTGTGTGTGTGTATGTGTGTGTGTGTGTGTGTGTGTGTGTGTGGTGTATGTATGT
ATGTATGTATGTAAGTATCTGTGTGTCTATGTATATCTGTGAGGCTGTATCGGTGACAGACAGTGTGAGAGTGTACGTCTGTGTCTGTCTCTCT
GTATGTCTATTCATGTCTATGTGTGTCTGTGTATGTTTATGTGTGTCTGTGTGTGTCTATGTGTGTCTCTATCTATGTTTATGTGTGTCTGTGT
GTCTCTGTCTGTCCCTGTTTGTATGTGTATGAATGTGTGTTAGAGAGTGTGTGTGTCTATGTCTATGAGTGTGTCTGTGTGTGAGTCTGTGTCT
GTGTGTGTCTGTGAGTGTGTGTCTATGTGAGAGTGTGTGTTTGTGAGTGTGTATGTCTGTCTGTGTATGTGTATTTATGTCTCTGTGTGTACGT
CTGTGAGTGTGTGTCTGAGTGTGTATGTCTGTTTGTCTGTGTATGTGTATTTATGTCTGTTTGTGTATGTCTGTGTGTATCTGTGAGTGTATAT
GTCTGTTTGTGTATGTCTGTGTGTGTCTGTGAGTGTATATGTCTGTGTATGTGTATTTATGTCTGTGTGTGTGTTTGTGTATGTCTGTGAGTGT
GTATGTCTGTCTGTGTATGCATATTTATGTCTGTTTGTGTGTTTGTCTGTGTGTGTGTGCCTGTTTGTATATGTGTGAGTATGTGTATAAATGT
GTGTGTTACAGTGTGTGTGTGTGTGTGTGTGTGTGTGTGTGTATCTGTGTCTCTGTGTGTGCCTGAGATTCAGTTTCTGGGAGGTGAACCCATC
CACCCTGTGCCCCGTGCCTGCCTCAAACCCTTCAGCTACTTGATGCTGAGGCTCTGGAGGCCACTGGGTAGTCCGTGCATCTGCCAACACACTG
GCTGGGGGCAGGATCACACAATCTTTTCTGCTCTGAGAGTGATTCAGCCATTCTCAAAATAAATAAGTACCAGAGTGCAGAGAGGAAGAAGTGG
AAAACATTGAAATCTGAGTCTGGAGGGCGGAGTTCTTCTCAGCAGGGCTCCTCAGGGGCAGTGCCCAATGGAAAGCCCTCAGGCCCTGCCACCT
GGCTCTGTGCAATTCTCAGCTTCTGCTGGAACTTCAGTTTTGTCATCTACAAAGTTGGGAGGAGGGTGTAGTGAGGGCTAAGGCACTGATGAAC
TCAACGCTTGATTCAATGTTTCCAGGAGCAGTTGAACTTGCAAGGCATCCTCTTCCAGCCTCTAGCAGGTCACCTGCTCTGAGGAGTGTGTCCA
AAGGGGGATGGAGAAGAGATGGCCACTGGCGGTGTGGGCGGAGGCGCTAGTGAGGACCAAGATGGAGTACCGCCAGAGGGAGGATCGACCCATC
CAGCCCAAGCTGCTGCAAAGGGACAGGAATAAATGCAGAGTGGAGTGGCTTAAGCAGGGGAAATCATCAAAACTATATTTCCCATGACTTTGGC
AGCCTTAGAAAAAAAAAAAGAAGAAAGGCGAAAAAAATCTCTAAACACACAAAGTGCTCAATTGTCCGAGCCTAATGGCCAACTTCCCAAAGCT
GGCCGGCCAGAGACCTTGGTGTTAATTCCTCACCCAACTGGAAAAAAAAAGATGGTGTGAGTTCTGTTCACTTAATTGCCAGAGCGTTGCTGAG
TGAGGCTGCACCCCCCCCCCTTGCGTATGCTAGGCCTTTCCTCATTACCTGCCCTACCTATCTCCACAATCTAGCCACAAACAGCTTTGGTTGA
CCACACCACTTCCCTATTGAAACTTAATACTGTTTGACTCATTCGGTGCTGCTTAACCCGAAGCCAAAGAAAACAAGGCAGAGAATAGCTCGCT
TGTATGGAGTCTGGAAAGCCCTGCTCGGCCCTGCTTTCAAAGTCCTATACAGTAGCCTTACTATACCTCAGCTGCAGCTGCACAGAAAATGGAG
GAAGCAGAGGTGACCCTCTGTGTGTGTGTGTGTGTGTTGGCGAGGGAGGTGGGGGTGGGGGACGGAGAAGAGAACAGGAAGGGAATGGGGTGCC
CCCCCTGGGGGGCTGGAAGAGACAACAGAGCAATCTGCCCAGTTCCGGGATTCTCTTTCTGGGCTCCAAAGTGTATCCTAACAGACAGCAGTTT
CTGGGCTGAACAGATACACTAAAAAGAAAGACTCAAAAACCCATTGGAAAAAGCGATTTTCCTATAGAAGACAGCAGAGAGAGGGGAGTTGGAT
CTCACCTGACCAGCTTCCCATAGGACAGTCCCTAGGTGACCAGCGACAACGCCCACCACTGCCAAATGTGGGAAGCACTCACAGCCAGAAGGTT
GTCTTGGGTTGATAGGGAGGTAGCCACACTGCTCCCTACTCCTTGCCTGAGATGAGGTCACCAGGGGGGCAAATGGGGGTGAGGGGTGGGGTGT
GCTTCCAAATTTGGAATGAGTACAACTCTCCCTTTGTAAGTGACCAGGCCGGTGCTCTGCAGGTACATGGCCGCTTGCTGATGCGTACTTGTGA
CTGGTAGACAAGATTCTGCTCACTCGGCCATTCCTGGATTGTAGCTGCCGCCCCTCCTCCTCCCCAGTCACACACCCCAGTGGCTGAGAGACAG
AGTCACCCCCAAACAACTTGAAAGTAACCTTTTCTTGAGTGTGGCACTCAGCCAGTGCTTTGCTAAAACACAACTGTGAACTCACAGGTGCTCA
GCGTCTGCCCTGTCCTCTCTCTTTGGTCCCTTTTCCTTGAGGCCAGCCTAGAGCTCCAATTGCCTCTTTTTTTCCTCTTCAGGTTTAGGGATTG
ATGGAGCCTTTTCTTTACCCCCTTTTCCTAAGCACGGAGGGAGAAAGATCGTTGTTGATGCTGCGAGCCGAGAACCCCAGAGAACGACATCCAA
TCAATCACTCACTCGCTCGCTTTCTCACTCCTCCGTCCAGCCAGGTTTTACAGGGACATCACTCAGCCTCCTCCAAGAGGCCTCTGTTTATCTG
GACTCTGTGCAAACATACTCAGCACCACTGCCAATCACATGTAGCATGGCAGGAACAGGAGAAGGACAGCTCATTACGACAAGTGAGAGATGCA
CGATTGCTCAGACCCAGCCCTGAGCATGGAGATGGAGGTGGGGGCCTCTGAGTGTCTTCTCCTTCCTCCCAGGCCCGTGCAACCTAGGGCTTGC
CCTGCTGAAGGCCCGAGTCCAGGTGCATTTCAGAGACTAGAGAGCTCTCCTGCCTCTGCCTGTGTCCTGCAGTCCTCCCATGGCTGTCTTCCCT
GAAGTCTATCCGTGTCAGAGCTGCTCCGGTAGATGGATAAGAACAGTTACATCAGCTTACTTTTTAATTACACTTTACAGAGGCGGGGTCCTGA
GACCGTGCTTTCAGGAGAGTTAGATAACAGTTGAGATTACAGGCTGGCAAAGGAGCAGGTCTTGCCGTGTGAGTTCCCTGCAGTTCTCCCACAG
GCAGCCACCCCCACGGAGGACCACTGCGGGTCAGGAGCACAAGATAACCAGACGGCTTCTGCTCTAAGCTACAATTACCTCCTTAGTCACCAGA
CAAAGCAGGAGGTGACGGAGGGTCATGACCACAGAGACAAACCCCTCTACTCTCAGGACACCAGGGTCCCATGGGAGCCACCACACCTGAAGTC
TGTCACTCAAGGCCCAGAATGGAGAATGACTGATAGAGACTGCTGCTTTTTTTTTCTCCCTGAATCCTGGGGCTCAAAGAGGGAGAGAGGAGTC
CTGGGGCCTCAGGGACCAGGAGACGCCGGGCCTGTCAGCCTGTTGTCTGTGACTGTCAGCCTTCGTCTGGATTTGGCCTCCGCTGGCTTTACTG
TTCCGTGTGTGGATCAAGGAATAGTTTGTGTCTGATTGAGAAATGTGAATGTGTATGTGTGCATGGGTGGATCACACACAGGTACACACACACA
CAAATATATGACAAATGCATACATATATGTGTGACTTATATGTGTGTGTGATAGATATAATACATATATAGAACATCTTATATGTATATGTGAT
ATAAACACATTGTATGTACATATAACATATATATGTGCTATCGTGTATATGTGTGTAGTAGACATGTGCTACATACATGTGTGTAGTATATATA
AACACATTACATATATATTACATATATGTATGATATGATAGAAGTATGTACTGTATACCAGCATGGTGAAGATATGAGTTGATAGGAATATATA
CACATGCACATGTCTCAGTATGTGGTACGTGTGTATGTAGATGCATGCAATGCACATGGTATAAGCATGTGGCATATGCATGTGTGCCATCAGC
ATGTGTGTGAACACACACACGTGCATCAGAGAAAAGCAGAATCCACTCTGGCTGCTGACTCATGCCAGTGTGCTGTGCTTGGCTCTGCACAAGG
CTCATGCCAGTGTGCTGTGCTTGGCTCTGCACAAGGGACTTGTCTTACTTTTCCGCATGATGAAAAGAATGGTTTGGTGACAGATTTGACTCTG
ACAGGACAGTTCTTCAGGGAGCCACACTCCCCAGCCTGAGACTTTGCCTCAGCAGGTATCTTCACACCAGTCATACTATGTCCACACAGCACTC
ACACTGCTCACACACTTAGTTCTGGATTCCTTTGCCCCAAATGAATTAATTCTACTTTCCTCTAGTCTTGGGAAGGAAGGGTGGATGGTGCTGA
TCAAGAATATCTGTTTGCTGGGTTAAAGCTGGTTGTTTAGTCAGCATTTAGCCTTAGCCGGATGTCTGCCCGGAGCCTTAGGTACTAAGGAGAC
AGCAGGATTCATGTCTCCATACACTCAACGTTCAGCAGGCACAGACACACCACAGTGACACAATGGTGCTGTGGAAAAAGCCACATGCTGAGGG
TGAAGCCAGTGGGTAGAGTTCATGCTTAGAATGCATAAAGCCTGGAGTTTAATCCCTAGCAATGTGTAAACTTGGTATAGTGTAGATACATGTC
TCTGCAGTACTCAGAAAGATCAGGAGTTCAAGCTCATCTTACATAGTGGTTTCAAGACGAGCCTGGATTACATGGCAGCCTGACACACACACAC
ACACACACACACACACACACACACACACACACACACACACACAGAGAGAGAGAGAGAGAGAGAGACAGAGAGAGAGAGAGAGAGAGAGAGAGTC
AGAGCTCTAAAGACTGTCTTAATGGTTAGGTTGGGGACAAGCTCTAAAGACCAGGTATTTGCCTAAAAGAAACACTAAGCAAATCGAGCCAAGG
CATCCCATGAAGTTGTTAAGAAGCAGCAAGCTTAGAGCCATCCAGTCAGCCCCAGCCGAGGAGATGCGGGCGGTCGGGCCCCAGACGTAATTGC
CATGGGTGCCCTTGGGAGGAATCCACCTTAGTCCTTTAATTCCAGACTTGCAGATAAGGAGCCCACAGAAGAGTCTGCAGGAAGCACAGACTTA
GAACAAGGGGTGCCACATGGGTAGTCCTGTCTCCTAGTGAGGGCCGCCTCAGCGGGCAGGAGTCAGAGGCTGCATTGACACCCTGGCCCTCAGG
GGCACTCTGAGCTCTGACCTGCTACATGTCCTAAATAGAACAATAAGCTCTGTATGAGCACCATGGTCCCTGCTGTGCTCACACTCCCTCTGAC
AGTAAGGCAGGTCCCACTTCTCCCGCAAGAATTCTGAGGAGTCTCTAACTCCATGCTACTCAACCTATGGGTTGCAATCCCGCTGGCAACCGTC
TAGCTCCAAAAATATTTACATTATGATTCATAACAGTAGTAAAAAGGTAGCAATGAAAAACAATGTTGCAGTTCGGGTTCACCACAACGTGACG
AGCTGTATTAAAGGGCCGCAGCGTTAGGAAAGCTGAGAACCACTGTTGTCACCAAGGCCATCTAGCCACAGCAGGAGATATGACAATCAGCACG
AAGAGCCTCTCTGCATATACCTAACAACGGTTTAAAAACAGACCAAAGGATCGCCAGTCTTCACACCGAATGGGGCCATCACAAAGTGGCCATG
AACTTAGAGTACAGCCAGTCTTCAGTAAATATCAGCCCTGCACGTGCCAATTCATTCAACAATGAACAGAAATGTTCTTCTTAGAACAAACAAA
CAAACAAACACATGTCTATTAAGTGGGAAGACTTTTCTTCTTGTTGTTCCCCAAAAGATACAATGTAAAAAAAAATCTGAGTTTCAGAAGATGG
AAAGAATTCAGGAAGACATGCGTGGGTTGTAGGTACAAATATGGGGCCGTTTAGATCAGAAGCTTCAATCCCACCGGCTTGGTAGTATACAGAA
ATCAACCAGTCACCCACATATACTGAGGTAGGGATTGTAAGTGGTATTTTATTCTCCAGCTCTCTGCAAAGCACAAGTCTGTAGGAGAAGGGAA
GTCATGGTGCTCATGCGAAATTCACGGGCCAGGAGGGGGAGCCCCTCCCCCCTCCTCTCACACATATACCTGGCACCAGCAATAGCGGCAGCAC
ACAGATCTTCCTAGAAACTGAAGTTATACAGGTCTTTACTCTAGACTTCCTAGCAAGAGTTTCCAAGTGCTCCGGCTATAGAGATCCCTCAGAG
GGCTGCAGACCACTGTGCCCCTCTGGCCCTCCTCACCCATGGGTCATACTGGGGACAGAAGTGGGACAAAGAGGAGTGCATGACCTGTGATCTA
AACTCAATTTGGTTGTGTCTCTGTATTTCTTCACTTAGGCAGGGAGGGGGTCACCAAAATCAAAACAACTCATTCTGGGTGCTCTGGGTGTCGG
ATGGGAGGACTGGGGCACTGAGGGTGGGTTGGGACCCGTATTCTTCCTCCATCTGGCTGCGTGAGATGGGCAAAACGTTAGAACCTCTCTGAAC
TTGGCTTGTCATCTGTCATTTTAAAAGATATGGCTGATTTCTCTGGCCCTTTTCTAGTTCTTCGGTGTAACAGCTGTGTCCTGTTTTCTTTGCA
TGATGGCACGAGAGGAAATGAAGAGACGAGATGAAACCATCTAAGTTCACATAACATCAGACAATCTGTAAAAACATACATATATAAAATGTAT
TAGGGTATATATTAGCATACATCATATAATATCATTGCATGTTTTAGGTATAGTACGTGGATTAATTTAAATGTAGTTTAATTAATTTAAATGT
TTGCTAAACTGAATTTCTCTCTCTCTCTCTCTCTTCCCCCTTTTACGATGCTAGCACTGAGACTTGGGGCCTCGTGAATGTTTGGAAGCAGTTT
CTCACTCAGCTACATGCCCAGTGCTTTGTAATATGTGAATAATATGCAATAGGAGACAGTATTTCTCAACATTTAATTAGCTGGGAGATAGCCG
GTTTGCTGGTTGGAACGCTTGTCTTACTGCAAGACAGTTCAAGGAAGGGACAGAGCTGAGTTCAGTGCAGTTAGCCACTGACTGGGACACAAAG
AAGGGCGACGGGTTCTCTACACTGCCCTTCCATAGACATAAAACTGCCCCAANNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNTATATGCCATGTGATCTATCCAAGAAAACCAGAAAAACTGCGGTCAGAAATAGGAGCTGACAGGTTGGCTAATC
AAAGGTTTCCAGGAAAATCTGGCAATTCCACCTCATTGCCTTGATAGAGAAGCAGATGCTCTATTTCAGTCATTTACAGCCGCAGACCACAGTG
GTGCCCTGGCAAAGGCAGCTGAGGTGGTAGTGAGTTGTGCATGCTATTCTGCTCTGTTTTTAACAGAAAGAAGATGGCAACTGACCTTATTATC
TACCATAAAATACAATAACGCTTAGGGGTGCTCTGATGGGCAGCCACCTCAGGGAAATAGAGAGACCTGTCTGGGATGGAGCCACTGGGCAGAG
TAGCATGAGTTTACTTTATTATATCTTTCTCTTCCCATTTTTTGTTGTTTTCTAGGCTGTGTGTTTATAATAACATTCTGAGCAGTGTTTTCTC
GAAATATGTAAAAATAATAATAGAATATCTGGGACCTCCTTTCAGCTCTGTGTGAGCTTCAGAAAGATGCACCCAGTACCTTGTGGTCACAGCT
TGAAGCCCCACTCTGGAAACCTTCACGATGGACTCGCAGTGAACTTGAAAGCCCAGGTTACCAGAATGTGTGTGTTTAGTCTATGTTACAGTCC
TCCGGTGGTTTGATCCAGTGTTCAATTCCCTGTTACCCTCCAGACCCCACTAATCACTAAGAGTGAAGCAGGGATTTCAGAGTGGGCACTTTCC
ATACTCCTGCATTTAAAATTCAGGGGGCAAATTGATATTTTAATAGCATACAGATCCAGGAAACTAAAGACATAAAGCATTCATTTTCCCCCAC
CCTGACAGTTTCCATCGGAAGCTCCTATGTTCCATGCAAAATATTGCTGTTTCCCACACAAACCCCAATCGTTTCAGAAGAGAATTAGGTCCTC
TTGATTCTAAATGCACTTGGGGCCACGCCTTGAACATGATGATGCCCTGCGTTCCCTGGGTGTCCTGGTGTTGCAACTCTCTAACACCCTGTGG
ATGTGGGGTTCAAGCTTTCTCTCAGGCAGAACCTCTCCTAAAGACTCCCAGAATGCACACGGAGAGAGCATGGAATGAAGTATTCCCTAATCCA
CACCACTACACCCAGCTCGGGACTTTCTACACTCATGCGGCTTAGGAAGCAGCTCTGAGAAGCCAGGCCCAGGCATTCCTGCCAGAGCAGGCAG
TCATCAGTTAATCTCTGTAAATGACTACGATTCTGGCAGGTGTCAGCTGCTACGAAAACAGATCACATATTAGGAGTCCTTTTTTTTTTTTTTA
ACTGAGTAACGAGGGAAGAGAAATAAAAATAATATGTCACGTGTGTTGGCCACATAGGGGCTTGTCCACTTGCCACAAACAGCTTATTCCTGTT
TGAGTTCTCAGTGATCAAGTCCCCAAATTCACAGAGCCAGATACTCAGATAGCCATTCTTCAAAGGCTGGCCAGAGACACCACCATTAACTGAT
GCTCTCACTTTAGAATGATCATGCACACAAGTGAGGGGGCTCATGCGCACGCGCGCACACACAGACACACACACAGACACACACACACACACAC
ACACACACACACACGCATTCCCTTCCTATGGCTACACACTGTCCTTCACGGTGTTCTTCCAAAATGCCACTGGGTGCCTCAGGAATGCGAGGCT
GCGACACACACAAAGCCTGCTGCAAGCTCTTCCTGACCCACATTCTTTCTGAGACCATACAAGCTATCCAGCGTCATGTTTCCCACCTTCCTCT
TCCTTGACTGGCATGAGAGACCACGGCAGACACTTAAAAGGGGCCGCATCACGCTTTAAATGGCTTTAGAGCTCAGATTCTTTTTCTGTGACAA
AGCCTGAGGTTTCTCAGGTGGGATGAGGAGTGGGGACTGATTGGGTCGCAAAGAAAAGAAGAGGGAGGATCCAGAGAGGGCTGCACACCGAACT
ACATATACTAACACTGACCAACGTAGAAGGCTCTAGAAAAAGAAGTTTTTGTACTTCTCCTCGTAAAAGGCTGGACGGCGTGGCTCGGGAACTT
GGCCTCTTTTGTCCATAGAGCTTTAAATACTCCGGCTACAAGAGGCCTCTCCCATGCGCCCCGGCCGAGCCTGCGTGACTCAGGGTGTTTGTGT
TTGAGTCCAGTGTTCTGACCTCTGAATGACAGTCCAGATTTGCCGTGGCGATACGTGCAAAGAGCGAGCGCTGCATGTGAACTACACAAATATT
ATGCTTAAGCTTTTTTTTTTTCCTCCATTTGGAGCCAAACAGGTCTTGTCAGTGATAAAAATTGCTGCAATATAGTGGGCCCTTTTTCTGGCCT
GGAATCTGAGACCGTGTCGGCAGTGCCAAGGAGGCCCATAGCAGGAAGAAATAGCAAGCCGCGGGGATTTGAAACCATAACACAACTTCTACGA
CATAATTTTATAATTCTAGCAATCGGGAACTAGGTGGTGGGCGGGGTAGCCTTGTAACCACTCCTGTGCTGGAAGAATGCTCCAGGGTCCTCCG
TGTAGCTCTGGCCTGGGCTGGGGTAGCAGCAACAGCATTTCTATGGGCACAAGCCAACGGCTTCCCCTCTCTTGTTGTGAGGCTGTGTACAGGA
GGATGATTGTGGCTCAGGCTGTGTAACCATTGACAGGAGAATGGCCCTGGCTCAGTCAACGGTTCTAGTCAAACAAACCTTGAGAACCAGGAGG
AAACAACAAAGTCGGCCTCTTAAAAGGAGGAATGGATCTCTACTGTGACTGTGTGCGAGGCTGGGCTCCTCTACAATCTCTAGTCTGCATAATA
CTTTGAAGGGGATGGAGAGGAAAAAAAAAAAAAAGCGGGAGGCCCACCCCTCCTGACTGGCCCACGTGGATGCCCCTACTTGCTGTTTCCTCGT
AACAGAGACACCTACCACCCTTACAAGACAGCAAGCATCCCACCTTGCTTTTCATTCCGAGGGAAAATCAGCAGACCAGCTGCCCACGCCCTCT
GAGCTCCCAAGGGCCTGGCCTACTAGGAATATTGGAACAGACCCACGCTGCACCTTTCTGCCTACTTCCTCCTAGGGATCCGCCCAGGGAGTGC
GCTTCAGGGAAGTAAGGATGCTTCTGAGGAGCCTGTACGAAGGAGGAAGGGGCCTGGGTGTGTGAGGGCAGAGAGGGATGCCCTGTGCCTGCCT
GCGTGTAACAGCAAATCTCACTCCTCCGCTGTCGGTTTGCAGCTGCACCCAATTTCCTGTTGAAGGGACACGCTGCTACGTTCCAGGGCCCACT
CTCGCACCTCTCAGACCTGCTGCTCGGGTTTTCAGGAAGGCAGGGCATTCTTGACCCCGCGAGGGCACCTTTCTTGGCTGCCACAGTTCTGTTC
CCAGCCTGAGGCCCACAGCCAACAAGCAACGGGCTTCTTACCCGGGGCCACCATCAGCAGTTCCGCTCACTAGTGTCTGCCCACCCGAGGGGAT
GTGCGGAGAGCTGTGCGCACCGAGTCCCAGACTGAGGCAACAGCCAGGGACCAGTCGGCTGAAGGAACCTCACCGCGCTATCAGAAGGATAGCC
CTGGTTCCCACAGGCACCCAGCCCAAGGGGCAATGTCCAACAAGCTCACGTTCGGCGTGAAGGGAGAGGCAGCCGAAAGATAAGAAAACTCCCC
TCCACAACACGTTTTCTGATCTCTTCCCTCCCTCCTTACCCTGCCTCCCTCTCCTCCTCCTCCTCCCTTCCCTACCCGGTCCCTACACTAGGAC
ACCGCAGAAACTAGATGATCACACCANNNNNNNNNNNNNNNNNNNNNNNNNNNNNNNNNNNNNNNNNNNNNNNNNNNNNNNNNNNNNNNNNNNN
NNNNNNNNNNNCGCAGAAAGCACCGGGGCTGCCAGGCCTTTCTGGGTGCTCTAAGGGAACTGTTGAAACCCCGCAGCATCAGCTGGGAGCTTAA
TGAGGAGAACGCTGTTCCATCTGCAGAGTATCTCTACTCTGAGAAGTGTGGCTGCTGAGCATGGGAGGGAATCGTGACTGTGGAAGCCATATGT
GTACATGGCTACACGCGTGTATCTCTCTTGGTACCCTTGCAGTTATCGGTTCCAGGTATCAAACTCCACAGGAGTTCAATATGTCCTAGTACTT
GCCTAGGCCTGTGGACATCATCCTGGAGACTGTAATCCGTTCTGCGGCTAGCATCTAACACGCTATTCATGCTATGTTGACAATCACCAAGTTA
TATTACGTAGGGAATCCTGACGAAAATACATTTCAATATGTGTACATTTAAAAAGCTGTATTATCAACCCACCTTCCGTTTAATCTTTAGCTAT
AGAGCCTTCAGATACAAATAGCCTCCTGTATAGAAGCTGACGAGAGGTTTTTGTAAGACTTGTTTTCTACTATGTTGTTTTCTTATCTACCCAT
TAGTCTTGAAGCCACGATACACACAGTTGCTTGCATGTATGTATCTATGTATGTATGTGTTGTGTATACATGTATAAACACACATACACATACA
AATCCAGCAAGGGGTTTTTAAGTTCTTGTTGCCTTTAGATTTTATGCTAAGTTTTCATTTAATATCTCTTCTTTCAAAATATGAAAGACGCATG
CGTGTGTATGTATGTGGTGTGGGCTGTATCAGGCAGAACATTTTCAGATCAGTAGCGTGAGTTCTGGTCACTCTGTGGGCCAGTCACCAGTGGC
AAACACGCTGTAAGATGTAATAACTGCACATACCCTCTGTGCCAAGTTACATAATCCTCACTGCATTTCCCCAGTCGGACGGCTGACGTCGGAA
TCTGTTTGTTCAGTGATATACCATTATAATAACCAAAAGGCAGTTTTCTCTGGGACAGGTGGGACACAGCTATTAATATGGCATGCTTACAGCA
ATACTCCTGCAGAGATGCAGGCCTGGGCAACTCAAATGAACGGATGAGCAGCCCATACAAAGAAAGGGGCAGAGGATGGGCAGCAGGAGTGGGG
CTCTGCCCTTCAGAGAAAGCAAAGACTAGTGTCCTCTGAGCTTCACAGGAGAAAAGCGCCCATCTACTTGATAAGCTTTTTATCTGAGCCGATT
GCTGTCTCTGGGGAGAAACACATGCAGGGCTCACAAATAGGCCCACATTTTCCAGAATGTAATATAGAAAGCCTATACCCCCAGGCATGGTATT
ACACTCAACTTATCATAGTACTTGCAGGATGCAGACAGCAGAATCAAGAGTTCAAGGCCAGCCATCGCTACGTAGCCAGCTTCAAGCCAACCAG
AGATACATGAGACCGTGTTAGCACAGCACAGCGCCTCTCTTTTATATGAACACACACAAAAATTAGGCTTTCTTCCTAGGTATGTTTAGAAAAA
GGCCACACCAAAATCCTTAGGTGTTTGTGTGTGTGTGTGTGTGTGTGTGTGTGTGTGTGTGTGTGTGTAATAGTGACGAATAATCACTCAAAAT
ACCCACACTAATTTTCCAAGGTCCCACACTGCAGCTCGGTGGTGAATTGACGCAAATGACACACTTTACGTGAGATGGATGGACTCTGGGAGAA
GGAGATATCCAAGGACATTTTTCTGCCTTCTGTTTACTCTCACTTAAGTTCACACACGTTTTCTCCGGCATCCCATGTCTAAAACCCAAAGAAA
AGCCTGTTCTCTTGCCCTCTCCTGGCTGCCCCGAGCGCCCGTGAGCTGAGAAGGAGGCTGGTCTTTTACTTACAGTCAAACCAAAACTGGTAAC
CGTCTTTAAAGCCAGTGCCAAACCATGCAGGAGCAAATTCAGATGGTTGCCTCTGCCTGCCTCATGAACCCGCACTGCTTCAGTTTGCCGTCTT
ACATACCTCGTCCCTAGAAACTGCAGAGGGTGGGTGGGCTCAAGGGAGCCCTGGGCCTTTGGAACCACCCACCTGTGCACAGAGGCATGGTCAT
CGACAGGTGGCTGCGCATCGCTGACTCTCCAGAACACTGCACTTGCTCTTAGAGTGTCCTAGGGGCTTGGTTGGTACTCTGAGAAGATGTCTGG
ACTATATTGTCGATTTTTTTTCCTCTCCACTATATAAATTTGATAAGCAGCGATTTACTGGCCTCCAGGCAGACACGCGGTTTCAAACCCCATT
GAAGATACTGCATATAAAACCAATTTCTCTTTCTTGTGTTCTCTCTCCCACCTTCTGCGTTTCTCTCCTCTCTTCTTTCTTCCTTCTCCCTTCT
CCGGCTCTTCTCTGGTATCTCTTCTTATAAGAAAGTCCTATTTTATACAAATCTAGTTTTGAGTGAACTCCTGATTTTTTTTCACCCTTGAAAT
GTAAACATTAACAGGGATAGTGTAACAGGCAAGTTCTTCACATTGTTCCTGGCGTTGCGGGCAGTTACTGCCTGGTCCTAGCCGTAGAGCACAC
GTGCCTACTTAAGGTGGGAATTTAAGCGATGCACCATGGGACTGTGGCTCTCTGAAATGGCAGTCACAGTTTTAAGAAAACTATCCCAGAAGTA
GCCAGGGCGGTGTCCCCTGGACACCACAGAGACACAGGAATGATTGTCAGCTTTAGAGGTGTCACTCACTGCAGGGGAGCCTCTGTTGAGCAGC
AGAAGACTGCAGGCTGGAGCCACCCCTTGGCTCTGCCAGCAGAGGATCATGGGTATACAATATCACATTTCCTTATCTTACCAAATAGACACCT
CGAGTGATCTGCACTGGGGTTCAGATGGAGAGGCAAGACAGTTACCATGCCACCAAACCACTGTTTTGTTTCTTCAGGTTTTAACTTTAAAACA
GCCGAGGTTAATTTCGACTTCCTATTTGAGCAAGTCTTTGAGTGGAATAGAAAGCTGTTTTGTCATGGCTGCCTGTTTACGCAAGTCAGGACAC
CATTGTTAAGTCCTGCCAAATTTCAGGGCTTTTCTCCGTTCCTTTTACAGCAGGAGTAGAGCAGATTACAGAGTTCTGGGGTAGCCGGGCCTGG
TCCCTCACACCTCCCTTTTAGGAATGGCTCCAAGCAACAGACATGCGTCATTTTAGGTCTTGQGCTCTGATTTCCCTGGGAACCATCATGAAGG
TGTCCTGACTTCTGCCTGGGGGACGGGGGGAGATGCAGATAAGGTGTAAAGATGCTGATTCCGAAATCCTTCAGACCAAATAAGTACTCTAAGC
CTCTTGCTGTAAAGTTTAACTTTCCTTGGATTCGTTGTGATGAAGCTGAGCTTAGACTTCGAGGTCTACACTGTTCTTTTTGCTCGGTAATCTC
TGGTGTGACTTCTAGACAGAAACACAGTCTCCCCAACTTCTTCCAGCTTTGTTACGGGGTGGATATGAAGTCAAATCCAGACCTCTCTGAAGGC
ACAGGCAGGAAGGTGGTGTGGAAGGACACACTTGTGCTCCTGGCTCAGGGACTGGTGTGCTACTAGGCCGACAGGCATGGAGAGGAGTTGGGCT
AGCCACTTGTGTAGAAGATGTGGACTGAATCTGTTTGGTTGTAATTGGAGGTTGATGCTGCTGTCGACGTTCATAATAACATCAGACTGTGTGC
TGGATGTCTTGCTAAGTCATGTTTCGGGGCATTTTATTTTTAAAGATGGGTAGAAAAGAAAGCCAATCAGAATACTGAAATTCTGAGCATGCCC
TAAGGGGGATCATGGGGTAAGGGGACTTCCAGCAACTCTTTCTTGGGGTAATCCCACACACATTTATGAGTCTCTCTCCTGTGGCCTTTTATAA
CTAAAGCCTTCTTCCCAAGTACCATGAGATCAGCCTACATGAGAGCAGATGCCCCCATCTCAAGCTCCACGTGACCGAGGAAGCCCACGTGCTC
TCAGGTGGCCCCCAGATTTCCAGTTTTCTAATGCATTGTCTTAGGCTCCTTCCAAGTCCCTTTGAAAAAGTTATATTCGCTACTGTCTGTGAGG
TTCTTAGAGGTAGCCTTACCATGTCAGGGCAATTCCCTCCACCCTCAGAATGTACGAACGCAAGGAAGCATTTCCACCTTCCAGAGTAAGGACC
AAACTCCTGCATCCAGAGAGCCAGGCCACAAGAGCATGTGAAAAAGGCTTGTCCTGCCCACCACCAACTCACATAGCATAACTAGCATAAGCAG
GGGGACACAAAGGGACCTGCGAGCCCTTGCCGCCCTGGGGTAAAGCATGGGACTGCTCAGCTTCGCTGCCTGCTTTTCTCTCCATTTCTGCCTG
CCCCCCCATCCCCCAATCTGTCGTCTGTGTGTGTGTGTGTGTCTGTCTCTGTGTGTGTGCATGCCTGCCTGCACCCACGCATGCGCTACCTTTT
GTGTTTGTCTCTGTCTTTCTTTGTCATATGCCTGTCTCCACCCC1AGTCACATACATATTTAAAGCCAGCGTGGGATGQGCCTGTTGATTTCCT
AGGCCATTTCCTTTGAAATGGCCATTTCATGCACACAGCGTAAAGTACTAAAAATTAACCCTTGCAGCTTGCTTATAGCACTAAGTAACTTTCC
CTTCAAAATAACTCCTGTCTGTGGCAGGCACTAAACCCGAAGAGCTGCAGGACCGGAGTTGTACACGCACAACACGCCAGAATCAGATGTTCAC
ACAATAAACAGGGCAGTGGGCAGCGGAAGGGAGAAAGAAGAACTGGATAAGAGAAAACCCTGAAAAAAAAAGAGTGCAGACTACAGAGCCCCTG
CTACATCCTGTGTGCTTCCCTGCCCCTGTCCTGTGGTATGTGTTGCCACGCCTCTGTCACATGGTATGTGCTGCCCCGCCCCTGCCACGTGGTG
TCTGCTCCCCTCTTCTCACCCCACAGTGAGAAGACAGAAGGAGTACTCCAAGTTTATAGAAAGAGAAGCGCTTCTAGGCAGCAACTGAGGATGC
CCTGTCATGTGCATATATATTCTTAGCTGGTGCAACACAAACTCTATGTCTAAAGAAAGTATGAATCTGCGGGCTAAAGAACTGTACCATAGAC
ATTCAGGAGAAGAGAAAGATGGGTGTCAGGGAGGAAGCGCCAGCAGGCCTTGGTGTTCTCCCTGTTTGGGCCCCTCATCTATTGAGCCTGAGGT
ACTAACATAACCTCCCACCTTAAGGATCGCCACAGGTGCCAGCACCTTCGCCCCTAAAACTGTTCAGTGTCACAACCCTGGTGTATTATGTATT
AACCTTACCCCCAAACACTTAATCAGCCCTTCAAAGAATCACTTACCACTTAAACTGACACTACCTTTGTAACCCAAAACCACAGTTTAATAGG
ACCTGGTTTCCAAAGCGGTTCATGACTGTTGCTCAAAGAGGTCTGCCCATGTTCTCAGGATCCTGTTAGTCCCTCGTGGATACCTTTTCTCATG
ACAAGGGAAAAGTCACTTACTATGGGACTTGGTCTCTATGATCAAATCGTCCTTCTGACGCTTCGAACTTCTCCCTAAGGGCTAATACTCTAAA
CCTTGTCCTCAGGGCCTTTCAGTGGTCTTTTGTGTATTTGCTGAATGGTGATGTTAAATATTCGAGGCTTCAAAAGGAACCAGTCCTTAGCTGC
TAAGGCGGGAAGAGTACAATCACAAACTGGCCCAAACTGGCTACGGCATATACTCTAAGATTTTTAGCAAGAAGTTTAACCTCTTAAAGCCTCT
GTCTTCCCATCTACAAAATGGGAATGGTAATTGTTCTTGTCGGAATGTCCTTTGAGCGGTCACTGTTATAATCCTTGTCGACAACACCAACTTG
GCTAGTTTTCCTGTGGCTGACACAACATACGGACCACAGATTTAGTGACTTAAAACAATGAGAGTTTATTCTCTCAAAATTGGAGGCTACAAGT
CCCAACATTAAGGTCACAAGAGGTGGCACTCCCATTGAAGGCCTAAGAGAAGGACTCTCTGGCCTCCTCTAACCCTTGCCAGCTCCAGGCACTC
ATTGGTCCATGGCTGCATCCTCCCCACCCCCTCTCTTCTGACAGCTCTTCTCTGTTCCATCTCTCCTTCTTCTTCTTTAAACGTCTCATGTTAC
TGGATCTAGACCTAGGCTACTCAAGACAGCGTCAGGCCCTTCACTTAGCTGTGTGTGTGAGAGACCAGACCCTTTGTCAGTGAGATCCTCGTCA
CAATCTAATGGTCAGAATATGGCCCTGCCCTTTCTGGTGCCACCGTTCAGTGCCAGAGATTAAAACCTGTTACATTGATGTCATCGGAACATTA
GCCGGTGGATCCCGGTCAGTCCAACGAGCCCCTTCCCATCTGTCCCTCAGAGTAGACCTAGTGTCCAGATAAATGTGATGTCATGCCTGTGTGA
CCCTCTCTTGTCATCTGCACTTCAGCATCTCAGCCCTTCATTTGCCAGAGCCATGTCCTGATTCTTCCATTCCCCAGCTCAGAGAATAGAGTTT
CTTTTTTTTTTCCTGGGCAAGATCAGCTATTTCCGAGTGTTTCTAGAGCACCCATACAGATGAAGCCAAAGGGTCAATGGTGGTCGGAAAGGGG
GGCTTCCATCCCCCTACCCCAATACCTAGGGTGTAATGTCAGAACACAGGAGGCTTGGAGCCTCCAGAATCCTGTGGCCACATAGACAGCAGCG
AGCACAGAAACAAAACTTGCCCTGATCCCCAAAGCCAGCCAGTTGCGACGTACTCAACAATTCCACCTCCAGATGAACCCAGGCCAGAGGCAGG
CATGTTGTTGTTTTGThGGACCAAGCAGCAGGCGTTAGCACAGAGTCTTGTCAACCAGATTCCCATGCCAAAAGGACCATGGTTTGCTTTGGGC
CCCGGTCATGCCAAGCCACAGATCACAGATCACACATAGCCTTTCAAGTCTGGAGGAGGGAGAGAATTTTTAACGGCAGCTGTGAAGCTTTATC
AAAGTTTACCAAAAGTTTGGCCCGAGAGAATAGTGTCAGGCTGAAAGGGCCACCTAGGCATTCTCCCAGTGCTGGGCTTTGTAAGTGCAGCCCA
AAGGCTTCATCACTCTGTCTTGTCTTGTTTCCCCCTGGCTCCAGGACACCCCATTCTAACTCTAGGGGACCATGAACTAAGGCACTTGGTGTCC
CTTATCTTCCAGACAATAGCCACCTAGTACCGAGTCTCTGCCCTGTTACCACACTTCTTGGCCCCACACAGACTCTATATTCATATTAAACAAT
TGATCGCAGAGCCGCAGAGCAGCCAATTGTTGTGGAGATTAAAGGTAGGGATCGCGGAAAGCCTGCAGACGCACTGTTTACTGACAAGCCTCCC
ATCTCTCCACAGAGTTTCCTCCCATAATCCCTTTCTGGCAGGACATTTTATGTGTCAATGCCGGGCGATTCGGTTACCCAGGCACAGTCAATAT
GACAAACTAATAAGGAGAAAAATAGAGTAGAACTCACCCTCAAAACAAACACAGATCACTCATGTGCCTCTCTGTGCATGCCTGAGAACACAGC
AAGAAAGAAGTGGGACAGGGACTGTGCTGTTTCCTTGTTCTGTTGATCTCTCTAACCAGGTGATGGAGGGTCTTCAGGCGGAGTCCTAGCTGAG
CCTGGCTCCTTTGAGCAGTGCTTACCACTTTAAAAGTAGCATTTTCAGAACAAACAGAACCATGTTTCCTGAGCTAGAATGAGCCTCTAAGCCA
CTGGTAGTAGAGGCTGGCCAGAAGGGACCCTTACTTCTGACTCAGGTTGAAAGGTCACAGAACCTGGGCCTCCCTCATTGCTCAGTGTCTCCCT
CGGGCTCATGCAAGGCTTAGAACCTTCCAGACATATTTGCAACATTTGAACCATCACTGATGGTTCAAATTCTGATCTCCATGGATCATAACCT
TATCCTTTTTACAAAGCTGGTGTCTTAGTTACGGTTTTGCTGCTGTGAACAGACACCATGACCAAGGCAAGTCTTATAAAGAACAGCATTTAAT
TGGGGCTGGCTTACAGCTTCAGAGGTTCCATCCATTGTCATCAAGGTGGGAACATGGCAGAGTGCAGACAGGCATGGTGCAGGAGGAGCTGAGA
GTTCTACATGAAGAGTAGAACTGAGAGTGCCACCTGAATCCTGCTAGCAGAATAGTGACTTCCAGGTAGCTAGGGTGAGTGAGGGTCTTAAACC
CATGCCCACAGTGACATCCCTACTCTGACAGGGCCACACTTTCTAATACTGCCACTCTCTGAGCCAAGCATATACAAACTATCACAGGTGGGTA
TTCATTTTAAACACAAAACTGCAGCCCTTTCCTCTAAGACGGCATTATGTGGGTGGAATCTCTGCTGTATCAGATGGGAAGACACCTTCCATGT
CATCCAACAACTATGGGATGCCGTCTGAGGACACCAAGTCTGTCCCTGGTTCCTTCCTGTCCCATCTTAATTCTTACACAGTTTCACTTGTTGA
GTCACTGAATTTGGGGTTCCAGGATGTAGCACAGTTAAACTCATCAAGGCTTGGGCACCGTGGTGACGAGTCAGCCCTGGGAGTTTGGCAGGGG
ATGTGCTAGAACCATGCTGCTCTGGAATGAAGAGACACCACTGATAGATTAAAGTAGAACTTCCCCAGGACAGGAAGGTCTATGAACTAGTCTT
CTCGTGGGTGGGATGGTTCACCCATTAGGACTGTCTCCAGCTTCATGGCAGGTCCTCTCCCCATCAGGCGTTCCAGGACCAGATCTGAGGCTCA
GAGCTAAATCTTCACTATTTCCTCTCCACACACTTTGAACGGCTGCTTCGGGTGGCTTTGAGAGAATATCTTCAGACAAAAAGGGGCAGAGGAC
TAATGAGACTGAGGACTGCTCGCCTTAGAGTTGCTGCTCACACAAGGGAAATGAACACGATGGCTTTATGGTGTCTCCTGGGACGACGGCGCTC
CCTCGGCCACTCTCCTTCCATTAGCACATCTCTCTGCACATATCAGATGGCAAATTACAGAACGATAGGGTTATTAAAATATCTGTAAAAGTCA
GCCCAGGCTCAAGGGCTTGACTTGGCCCTCCTGCCCGTTCGGCGGTTTATTTACATCTCCCAAGGTCAGCTCGCCAGCATCTGTATTTTGCGGT
AAACCGCCCACTCCGCAGACCAACCAGAACAGCCCACTTTTGAAGATGCGCTTCCCTTGGTTTTCCAGTAAGCCACTGGCACTCTTAAGAGTGA
GCCCAGAGCTCTTCAGACCTAATCCTACTGTCGCCCCCCACCCAATTTTAAAACTTCAGGGTAGGAGCGTGACCGTGTCTGCAGCCAGATTATC
ACCAGAGTAGTTCAGGCAAGAAAAACTATCATAATGATATTGTTGGGTTCACACATTTACCAAAGGCAATTATATAAAGCCTTCGTAGTTCATG
TTCACAGAACAGATTGCGAGTCGTCTGGTGAGGTCACAGGCAGAGGAAATACCCTCTACAAGGTTCAGGTGGCCCACTCCTTCTTTTTCATAGT
GCCTGACACTCCCCATCCCCCCATACATCTTTCTCCAGGGGGCGGGGGGGGGGAGGTGGGGGGGATGCATATGGGTGAGCACACACACACATGT
ACTCCTGAAAGTCGGAGGACAGCCTCAGGTGTGGGAGGACGGTCTCAAATGTCAGCCTCCAATAACTGCCACATTTGCTTGAGACAGGGCCTCT
TATTAGCCTTTTGCTTTGCTCAGTGATTAGGCTAGAAGGCCAGCCAACTCCAGAAGTCTGCCTGTCTGTATCTCCCATCTTGCTGTAATCCTTG
AGGTAACCGCCATATGCTGCCACACCCTGTCCTTGGGATGAAACTGAGGGCCTCATATTTGTGAGGAGAGAGCTTCACTGACTGAATCTCCCCC
TGCCTGGCACTGCTCTCTACAGTTTCCTAGGCCTTGCTCTCAGGCAAGTGAGTCACTGCTTGGTCCTAAACATTCTGTAATTGTGGGCACACTA
TCTTGAAGGGATTAGAGTCACCCCTAACACCCTCAGGGAAACCAACCTCCATGCCCTAGAGCCCCCACCCCACCCTGCTGCTTCTCATTGGCAG
ACTTTTGTGGTGCTGGTGGTGGTGGTGGTGGGGTGATACTTTTTGCTGTGTGATCAAGCTTAGAGAGGCTAAGAGCCACCATATGTTGCCAAAT
GAGAGAAGCCACTGCCCAGACATGGATCTGTCTGATTTAATATGATTTTCCATAACTGACAAAGCACCGGTTTGAAATTGAATTTGTGTAAAAT
TACAGATAGCCAGCTCAGGTGGAAGGGGAAGGAAGCCTTGCTATTCATGTTCCCGTGGCTTTTCCCGGTTTCCCCATTTCAGTCCTTCACGCCT
TGGGGGAGGGGAGACTCGGTGGACGGCCTACGGAGGGTAGCTCTTGCAGTCAGACTTTCTCCCTGGCCAGTCATGGCTTCCTCTGAGGAGGACT
CCAGAACCTGGAGATGTTGGGGCGAAAGGACAAATCCTCTGTCCATCCATCCATCCATCCATCCATCCCTCCCATCATCCAGCACAGAACTCAT
CTTCACACCTGGTCATCAGAGGGGCTCCAGGATAAACTTGGGGAGAGGGGGTACCTAGAAAAGCCTAATTCATTAGGACGGCCTCATGCCAATC
CAGAATGCTTTGGGGCTCAGACTCCGACTTTGTCCACTTTCTAGTTGTAGGTCCTTGAACGAAGGAAGCACTCAGGTCCCTGTGCCACACTGTT
GCCATCTGTAAAATGGGCACGCTCATAATGCCTGACTCATGAGATGGTCTTGAAGACGAAGTCAGCATGATTATAGTATTGGGTGGTCAGCATG
CTGTGTCTTGGTATGGTTGTGTCTTGTTCACTCCTGCAGAAACCCCTCACTGAGGCTGAGATGCGATCCTGGGATGTGTAGAGCACAGCGCCCA
GCGTGTCTAAAGTTCTCGACACTGCTCCCGCCGTCTGTGTAACCATAACTGCCATTCCTGCTACAGCTCTTGCTGCCGGCTCTCCGGCTGACAT
CAGGCCTGTCCTGCGAGGCTCACAGCCACTCTGTGACAATGGCCTTTAGAGTTTTGCTGATTTCAGTTTGGGGTCCTTGGGTAAACTCCAGCGG
GACCATCCATAGTCACTATCCACATCTTTGCCTAGCGCCTCTCCCCTGCCCCACCACCCCACCCCACCCCACCCAACACCCTGAAGGTCCAGGC
GCCCTCCATAACTGAGCTCTCCCAGGCTGTCCTCTTGTACTGAAGTCGGTCATATGGGGAACGGCTCATTTCCCCAGCGCTGGCTTAGCCTTAC
TCAGCCTTTTCCCTGTAATGGGATTTTGGCTCTGGGAGAGGGCAGGATTTAGTTTCTTATGGTCCCTTTTCTTTTCTTCGCTTAATGTTGAATT
TTATTTTTAAAACGTTTTTTTTAGAAGGAATAATTAAAATAATATATTTTAAAATGGGGTTGTTTTTTTGTTTTAAATAAACAAGGCCATACAC
GCTTTCTCATCCTCTTCAGTTCACAGCAAGCACTCCCAGACTGCTAACAGCTGTAGTGAGCTAACAGGTCACTTCCTGAAACCAGCGGCCCTGA
CTGGGCAGCGTTCAGGGGGTGGTCCCTCCCCCTCTCCGTTGCTTTCTTGAATTAGACTCACCATATATGAGTGGCCTGACTCTTCTGTGTTTTT
AATGTTACCGTCTACTAAAGGGTTTCTTTGAATGAGGTCTCATCTCCCTGACTGATTTTAAGTCAATACCTCCCTCTGCCAAATCCACCACTCT
AAGCCTGCTCTCAGCTGCAGCCCAGGCTGTCAGACCCAGAAGAGGACAACAAACAGTGTGGGAGGTGACACAGCCACAGCTGTGGGAAGGACAG
CCACTCAGGGCAGGCCCTAGGCTGGGACAGGGGGGAGGTGCCGAGTGACAGTCCCCTCGACCAGTTCAGAATGGTCCTGCCAGACTTATGTCAT
TACTCCCAGAAGACTGCATGCATGAGCCGAGACCTGTATCCAGCCAGAGGTGCCCCTGTTACAGCCAAGTCTTTGCCATGAATTCAGAGTCAGG
GCCTTGAATGGGTAGATCAGAGACTGTTTTGGAATTTGTAAGATTAGCCACCACTTTCTTCCCCCCCCCCCCCCGCCCCCCTCCCAAGCTACCA
GGGAACAGGATCCTGCATAGCTGGTGGCTTCTGGCTCCCTTCCTTAACTGAGAGACAGACCACTCCAGCATCCTGGCTCTTAAGTGATATTTAC
TTAATAGTTAACTGGGTTTCAAAAGAGAATGGGGACTTGACTCCCTTCACTAATTAGCCCTGCCGGCTGGCTTGTCATCTCCACCTTCACCACC
CACGGAGTGTGCTCTTGACAAGTCATGACCCGAACTCCCAGGGGACTTGCACTCTGGGCTTGCCTCCTCCACTGTCTGAGAAACACAAGTCTCT
AAAGAAGATAAGGCTAGAGCAATGAAGGGCATTCTGGGAAAACGTAAAGGATCCCGCCCCCCCTCCCCAGATCCCAGAGAAGCAATGGCAGCCT
GGAGGGGAAAGTGACTTGCCACAGGTGGCATACACTGTTATCCGAGTGCTTGGCTGTCTTCTCCGGTTCGQTCGCTGGACACATTCCTGGTATT
TGGAAGTAGCACAGGCACCCTTCCTGAGGAGTCCCTTCCAAACACCATCCAGGCGAACGAGGGGCTGTTGGGACAAGATAGGGCAGACCCACGT
GTGCCTGTGCACAGGGCTCCACCCAATTTCTCATCCTTATATTTTATACGTGTGTGTGTGTGTGTCTGTCTGTGTGTGTCTCAGTCTCAAATAA
CCCAAACAGGCAGGGTACTGATACTGCAGACAGTCTGCAGGCACCTGCCCAAGANNNNNNNNNNNNNNNNNNNNNNNNNNNNNNNNNNNNNNNN
NNNNNNNNNNNNNNGGAAGTCCTAGGTCAGCCAATGCTTGAATTCTTTTCCTTTCCTGTCCCCTCCTCCCTGCCCACTCCCATCCCCCCCCCAA
TTTAATAAATTTATATTTAGAAACTAAATATAGTTTCTAACCAACAGCAGCCCTGTTATGAAGAACATCTTGCAGCATAAGGAGAGACATCTCT
TAGAAAATCAATTTCCATCTGTAGAGACGTGGGATAAGGAAGGGAATGTAACCTTTAGCCCACCACCACTCAACTCTGGAACCTGAGCCCATCC
CCGTCACAACTCATCGTTGTGTCTCTTCTCGTTCTTTCTTTCCTCTGCTGACATGAGAAAGGGAATCACTCACTGGTGAGGTACCAGACATCAC
GCTACAGTCCAGCCTGCTTCTCCCACAAGGAGAGGCAGACTACTGAGCTGGCTATGGGCAGCCCCACCTGTAAACTCTGCAAAGCCAGCCCCTT
GCCCCAAGGCTCTGACAGCAATAGATAGAGCCCAGGTTGGCTCCCCTCACGGTTGGTGTCCCTGAAGGGGGATTGGTCAGCCATGAACTCATCC
TCACAGAATTGTATAGGGGAGCCTCAAAACAAGGCTTCTGCAAACTATCCCAGGCATTCTGTGATGTAAGAGCCCACGATATGGAGGCCATGTA
GGAACCCTTGGTGCTTCTCTTCCCCTCAACCCCCCACCATGTTGCCACCATGCCTCAGGGACTCAGAAAACTCACCAGTCTCCCAGTCAGCTGG
CAGCTTAGGTGCCTGTGGCCATCTTTCTCAGAGCTACAAAGAGCTCCCTTACACACCAAGCTCATCTCTGAGCACTACAGGCAAGGCAAACACT
GTTAGCTCATGGGCAACTGAGGACTGAGGAGACTTGAAGTTCCTTGAACTTCACGTTCTTCTTGTAGCATAAGTCATTGTTGCTCGGCCATCTT
GCGTCAGCCATCTTGCATCAGCCATCTTCCGTCGGCCATCTTGTGTCTCTATGTGGATATCACTGGACAGGAACAGCTTTTCAGACACGGCAGA
ATGGCCACAAAGTACAGGAATAATGACGCCTCTGACCTCAGTGTGCCTTCTGCTGCTATGACTCTTGAGTGGCAATGTAAGTGAGGATCCTGCA
GCTTCCCCTTGGCTCCTTTGCCTTCAGGTGACCATTATAAACCCCCCTAGGCCCTGTGCGTCCACCTGGTCTGATCATGTAGCCTTCAAAGGCC
ATGCCCACGGCCGAAGAAGCTATTAATGCCTTTCAAGCCAACTGAAGGCCAAAGACTTGGCGCACAGAGCTCTTTTCGCTGCCCATCAAATTCC
CTTCTCGAGCAATGCCTCCATTCCTGTGGTGTTCCAGGCTCTCCCTGGCTCCCTAGTCCATCCAAGCTCTGAAATCACTCTCAGCCAGTCACAG
GAGCAGGATGGAGGCAGGGAGTCTGGGGAAGGTACCAAGTGGATGTGAGGTTAGCTTACCTGTCTTCCTCTCTGGCTAGTCCTGTTCCCTCCTC
ACCCTGTTCCCACACTCTCCCATATTTCAGCAATCCTCACATGTCCCTTCCTACAAGGACCAAACTGAGTCCTCTCCCAGTTCCTAGACAAGCC
TGACAGTCCCTCTCACTGTAACATCTCCCCATGTCTGCTCTCTGGTTATACTTGACAGGTGGTGGCGCCAAGTGCCTTTTCCTTCACTGTCCTG
AGTGAGGCGTCATCCTGCCCAGTTAACAACCCCACCCTGCTAACCGGAGGACTGAATCTATGTGCTCCAGCATGGCCTTCCCCACACATCTGGG
CTCTGAGACCCACAGAAGGGTCAGCAGAGTCCCAAGTACCCTTGAACACTCCTTCACTTCTCTGGGTCCTCAGGAATACAGGCCACGAGGTACA
GAAACCAGTCAGTCCCTGTAGAAGCATTTTTTCTTCTGATCTTAGGAACACAGGTCAAAGTGGGACACTGGTGGGTGTGAACCCCATTTCAAAG
TTCCAGGCAGGAAACAATGCAGTGACTGGGAGGTCCCTTTGCCTCTTGACATTATGGCCCATAATGTTGTGGCCTTCGATACCCGCCCCTCCCC
ACCATGCTGTTTCCAGACCCACACCAAATACTCCTCCCATGACACCCTGCCTTCCTTCCTCTGCAGACAGCTCAGCCTCACCTGACGCAGACTT
CCACATATTTGAACCTGCTTTGCATGCTTAATTAGAGACCTAGGCCCTGAAATAAGCTGTCTTAGTTACCCACAACTTCCTAGGGATCCCAGCC
CCTACCACAGCACAATACTGCCTCCACCAGCCAGGTTCCTGCTTCCTGCCCTTCAGGGGTGTGTGTGTGTCTGCTGCTTCCTTCTCAGACCCAC
ATTTCTAGATTCTTTTTCAAATCCATCCTTCGAAACTCTTTATTTAGCGCTCTCTCTCTCTCTCTCTCTCTCTCTCTCTCTCTCTCTCTCTCTC
TCTCCCCACACACACCTTTTCACCTTAATAAAGCCTTGCCCTGGCGCCAGGGCTGGAGCTCACCTGGGAGAGTGCTTTCCTAGGGCTGTGAACC
CTAAATTCTATCCCCAGCACTTGGCACCCACCGACATCTGTACCACCTGGATTCCAGCATTCAGGAGGTGGAGGCAGGAGGATTAGAAGTTTAT
GGTCATTCTCAGCTCTCACGTATATAAGTTCAAGGCTAGTCTGACCACAAGAAATCTTTTCTTTAAAAATAATTGATCCACTGCCAAGTCAGGG
GGCTTCAGGGCCCCGTCCCCAGCTTCTGGGGACTTTACCTAGTATGACTCCTACCACGCAGGAAAGTCAGGCTTGTCTTCCTCTCATTGTCCTT
CTCTGGTCACAGGATGGTGCTCGGACAGCCAGCCAGACGCTCTCTCTCATCCACCTGTCTCCTTACCCACCATTTGTGTGAAAGAGTGTCAGAT
TGTGGCCCCAAACAGGAAGCCCTCTGGCCCTCTGGCCCGCTAATCCCCTTTGGGAGGAACCTTCACTAGGTACTCTTAGAGGGTCTTCTGTTCA
AATCAAGGGGAAAGTGGTTTGAATGAGCTGTGTTTTGTTTTGTTTTGTTTTGTTTTGTTTTTAAAGAAGACAGGAAGTTCCTGTTTTTTAGAAA
GGCTGTTCATTTTCAAAGAACCAACTTACAGAGCCAATCGACCATAGGATTGCTGCTGGATTTTTTTCCCCCAATGTGGTACACAATACATGCG
AAAAAGAAAGCGGCGACCCCCACCAATGAGAGATAGTCAGTAACACAAAGGGAGATATTATAATCGGCTTTTGTTGGTGCTTGCGTCTTCTTGT
TGAAAACCAGAAAGCCCCTGAGGTGCTGAAGTCCGGACAGAAGCTGATGTTATCGGCCCCCAGAAATCCTGCCTGGAGTCCAGTGGCAGGCAGG
ATCCGAAGCTCCCAAGACCACAGCAACTGGCCCTGCTCAGTACAAACGGTGCTTTCAGTCTCTGGTAAACAGCCTGTGCCCCAGGGCTCAAATC
CTTCAAAAGTTTGCCTAGQTAATTGCCTTTCATTGCTGTCTGCCCTGCGCTCGTCAGTCTAATCTGGAGGTTCAGACAGCCAGGCCAAAGCTTG
CGAGTCTGGAGGGGAACAAGCCCATGAATCTAATCCACTTGAAAGACAGACTAATACAAGTGCAGCTAACTCATTATTTTAAGTGATTTGAACA
GAAGGAGTATCGCACTCTCTATAAAAGGGTTTCAAGTCAGTGCAGTGCAGTGTTTGGCAGCTTAAAGCCATTAGAGGAACTGCAAAGGGTGACA
GTGTGACTCCCCGTCGCCGTCCTCATTTTATGAAGCCTGGTTCCTCCCTGTGACCACCCTAAAATGGTTTTTTCCTGTCCTGCCCTTGGTTTTT
GAGTTATTTGTGAATTATTATAGTAAAAGACTCCAACTTTTGTCTTTGATCCAGGCGTGGGTACGAAAGGAGACACAAATGTAAAGACAGAATT
CTGAGGGACCCCTCGGACCTCTGAATACCCCCAGATCTGAAAATAGGAAGAGCTACCCTCTTCAAAGCCCCAACACCCTCAGCACTTAGAGTTT
TTGAAGGTTAAGCCTTCCCATTTTGCCGGTATGATCATTAGGTAGGAGGAGGGGTGCTTTATGGAAGTAAACCTTCATCCTACCACCAACCTGT
GTGACGAACAGAGACCGTCCCTTCATCTTGCACTCCAGCTTTCTCTGAGTTGGAGCTGGCACAGGTAGCCTCTGGGGCTGCTTCCTGCCCCATC
ATTCCATGAGTCTTTGGAACAAGACCTCAGAACTGTTGACAACAATCTGCACCATTATCTAGTTACATGTGACTCATGCCCAGAAAGCCCAGGA
GAAGCAAGTGCTGGCCTGTCCCTTCCAAGGTAGCAAAGCCAGTGGTCAACTCAGGGGCAAGCTTATTGGCCATTTACACCTGTGGCTCCCCCGG
GGCTGGATTCACCCAGTGCCAAGTCTGCGTGTCAGACTGAGACTGTGACAAACCAGTTTCTGCAAACACCGATGTGGAGACCAGGTTCTCCTTA
GGCGCTGCCTAGCCATGGTTGCCCCCACCTCAGCCCCTCAGTTCATGTGTCTTCCACCCAGGCCACCGTGCTAGATTACATCTCTCGAAATCAG
AGCAGCCTCTAGAAGTCACATGCTCTGCTTCCATGTTGAAGTTGGCTGAACTACCCTATTGGTGTGACGTTCCCTGGGTCAGTCTTCCCTTGTC
ATTTTAGTTTCCCTTGACTGTGGGAGCTGAACCATTCTTTTACGGGAACTTCTTATGCACTTAACAGCTACCAGACAGCACCCCTGGTCTCTAC
CCACTCAATGTCTCTGGCTCTCCCGACTCTAATCAAGAGTAGTCCTGTTTCCTGACATTACTAAAAGTCCTCTAGAAGAGAGTTCCCTACTGGT
TTAGACCAGCCTGGGAAAGGCAGAAGTGTTTAGTCCATGCGTATAAGATGTCAATGTGCAAAACTCAGTTGATTGGGTGTCTGTCTGCAGATCA
CTTCGGCCTCTGGGTCTCTGGCACCTCCTTCCTCCACTGGCCAGTCCCACTCACTGTGTGATCAAATGTGTCGGTATGTCCCACAATCTCAACC
TGTACATAGCTTTTTTTCTTCCATCTGCGTCCAAGTCACTGAAAACCTATCGCTTCTGTATGAAAGGCTGCCTTGACCTTGTAAATCAAAGGCG
TTCTCTGGAGGGAGCTCTCCCACTGCTGGGCTCCGTTCGGTATCATCTTTCTCAGGGTGACCTCATCTATTCAAGTGTGAAGGTTGTATTTGAA
ATGTCATTTACTTTCCCTAGTCAATAAATGAAGCCATGGATCTTTAGATCATCAGAGGCCTCCTTATTACCGAGAGTCTCTACGCTGTATGGGT
ATGTGTCTTTTGAGATCTGTGTTAGAGTTTAAAAGAATTCTGTCAGACTTCCGTTTGCCTCTCTGAATCAGAGCACGGGGTGGAATTCCTCTGT
GAGTGGAGGGTGCCCCCAAATGAGTCTGCCTTTATTAGTTTGTGTCCTTTGTTCAGAGCACCAGGGATTACTTTATGAAAGCACGCGTGACTCT
TATCTTCAAAGGTCTCCCCTGCATGGCAGACTCTACCTCACCAACCTGTGTTACTAATCCATTGCCATCCCAATTAGTTCAGATCAACGTCAGT
TACTTGGGCTGTGCAAAGGGGCCGCAAATGTAAGTGAGTCAGTCCCCCTCCTGTGGTTCTCCCACGAAAATGAGAGATTACAGGCGGGGAAACA
TAGAAGCGAGAACTGTCTGGTGCCTCAGTGTTTGAGAGTGAAGTGAAGACCTCAGGGAACCCTAAACAAGCCAATGCTTGAGTAGAAATACACG
ACCCCTGCTGCGGGGAAGTTTCTTGGCCCCGTCTCCCCACCCTATTGCTGATGGCAAGCATCTGACAAATTTAGGAGCCTCGGACACACAGTTG
GGTCTGACAACATTTGCTGGGTCCACGTGGAGGAGGAACCTTGTGTTTATGCCGCGTGATAAGCACCGAGCCTCCAAAAGGCACATCTATGGAA
AAGAGGCTGGAGTTTGGTAGGTCCATAGGATGGCTCATGATAATTCCTAGCCACCCATCCCACTGTCATCTTGAGGTCATCAGCAGAGGGATGG
TAAGGTATGTGAGGGTTACATCTTTCTAGGTGGACAACAAGACCTATGAGAATACACTTCTCTGATCTACCATTGTTTCTAGTACTGTGGGGAG
GCCATGGAAGATCAAATCCAAGTGCAGTGATTAATGTCCATTCCATGGACAGTCAGACAGGACAGTTCTTGTGAAAGATGTCTTAGAACAACCC
AGATAGGAACAGCCAGGAATCATGAAAGGCTGACCACCCCCCACCCCCAAGCAGGAAAACCCTACACTTATTTATTAGATTCTTCTGATGCAGA
GAGAGACCTGTGCATAGCCTGGCTAGAGGATGGGAGAGGGAGTTCCCCGCCCCCACCCCACAGCTCTCTGAGGTGCTTCTCTTGGGCTTTGCTT
TGGGGAATTGATTCTAAGAGGACAAGTCTGGTGTGTAGGAAAGGAAAACACACCAGTGTCCCAGGTTCAGGCCAGAGAGAACAGACATTTAAGA
CCAGACCAGAACTCTGAACCTACCCCTCCACACACACACACACACACACACACACACACACACAACGCACGAGGAGAAGAACATGGGGGCAGAC
ACTGACTGGATCTCTCTTGCAAACATTTACCAAAGGGGGCTAGGCATTTCACCTCTGCGGAATTTTTCTATTTCCCCTGTGTCAGAATGTTCTT
CTGTGAGTTGAGAGTGTGCAATGAAGGCTCTTAAAGAAAAAACAACCCTTTGACATGACTCACACCTCAGAAGACAAAGAACAATGAATCCCTG
GCTTGCAATTCAGCTTTCAGACCCTGTGTCCTGGCAGGCACAGCGCCACCCTGCCATGAACCTGCGTTCTGCAGAGCGGGTACGGACTCCCTCT
GGAGCTGTGGTGTTCAGGGAAGAGACGCTCCCTCATGCTGCGTCTCCGCGCTGGCACTTAGCCACCGGAACCACACACGCATCGTTGTGGGAAC
CACGGTTTGCACCCTGGAACTCTGGCTGGCTGGGACAAGTCCTATTCCTGGTAGCCTTCCGGTGTCTCTGCTATGTGGACCACCGGCAGTGAGG
ATAAAGAGTCTGTGCTGGACAAGCCCAAGCAGTCCTCACTCTGGACATCACTACCATTCCCTGAAAGGGTAGCAAGGGGAGACTCTCTTGCCTT
TTCTTACTACAACTGAACTGCTAATGGTCAGGCCAAGATACTATCTCACCCTAACAGTACCTCACAGGCTGATGCAATGACCCCTCCTCACAGG
AGGGGAGTATGCCCACTTGCCTCAACAGTAAGAGCCAAACTGGTGTCTCCTTTGCAACCTCTCAGACTGGAGAGTCATGCCCTTGCTCTCTTGT
TGCCTGAGCACTGTTGGTCCGGGAATAGGGAACACTGGAGCGGTACCCAGATACTAGAGATGCCTGGAGCCAGTACAAGCCACAGCACAACCGG
CATCATCTGTTCTGTTTTGGGGAATATTCTACAGGATATTGCATTGCATCGTCTTTTCTGTCCCCATGAATATCAATTTCTCTTAATACTCTGC
GGAAGAATGAACGCTGTGATCTCTGCTTAATACCCTGGTCAGAGGAACACAGGGGACAGGGCCTGAGTTCTGTCCATTAAGCCACCTTAGAAAT
TCCCAAGAAGTTCTAACATTTAACAAAGTGACTAGTACAATGCTAGACTACAAGGTTGAGCACGCACACGCCATGGGGCTGGCGTAGACGTGAC
CTCTGCTCTCCCACCAGGGTCCCGAGTGTAGGCTCAGTGACAGCCCTTGGCACAGAGATCTGGGAGCTCCACAGGTAGCGGTGACCCCCAAAGG
AAACAAAGCCACCTAGTGGCTTGATAGAGCCTGGATTAGGTCGGACTTCAATCTAGGCTGGGCCCTGATTGACATTGTACAGGTAATAATAGAT
CCATCATCACCACCCTCTGCAATTCCAAAGAAGAATAGCCACTCCTTTCTTGGTGCCTACCATCACCCTAAGGCATGTTAAGGACCCTCATAAA
GTCTCAGCTGTTCACTTAATGTGACCGCCCAAGGGCCAGCTCTTACTGCATCCAGTTTTCAAAAGACAGTTGGTGCTCCCCTGAAGACCCAGCA
GCAGCCCAGAGCAAGCTGGACAGAACAAGCCACCTTCTGCAGGCAGCCCGGACCAACATCTCCAACATCCCCTGACCTGAGCCACACGAATAGA
GCCTGCAGTGTGTGCTACTGTCAGTACATTCCACTCTGTAAGACACCCACAGGCATGAGGTCCTCCCGCTCGCTCGCAGCTTCCCACACGAGGT
GTCAACAGCTTCTGTATGAGTGACGGTCTTGTCCCTCCATACAAAGGGCTGAGCCCAAGCTCCCAAACTAAACTACCTGGGGCCTCGGACCCCT
GACACTGAATTTCTCCTCACCTTATTCAAGGGAGGAGTAAGGACGAATATGAGGCTGGAGAAGAACCACTGGGGGCTGGGGACAGCAAGTCATC
TTTACCCTCAGCCTTTCGAGACTCTATAGAGGAGAAAGCAATGGAGGAGAGGATGATTCAGCAGCCGCTCCTAGGATGGCACCTGCCACATGGT
GGCTGAGCCTGACTACAGGCCTAGAAAACATAAGTGGACGTCAGAGGTGCAGGCAGCTCGTGCAATGCGGTGAGGAGTCGGGGTGGGGCTTCTT
TAACAAAGTTCGGCGAAATAACTTATCTCCAGGAAAGGACACTCCACAGACCAGACGGGGCCTTTCGTGCCCTCAGAAGCACAGTTCTGTCTGG
ACGCCAAACGCCACTCTGTCTGGAGGAGAGACGGTGCCAACAGAGAATGAGAGGTGTTATGAAAACAGCGTGTTTTCTAGAAGCGGGTGACTTA
TCCCTGAACCCTTGCACAGGGAGGACTCTGCCTAAATACAGTGCCCTTTCCAGACCTGTCTCAGACTGATCTGGCTGAGAGGAGCGGCCTCCCT
CGTGGCGAGGGCTGACCTATTTCCCCTGGGAACTCTCTGGCAGCTCTAATGAGGAATACCACTTTGCATTTATAGAGAGCCTTTCATCTGTGGA
TCTCAAAGCTGTACCACAAACATTAATTAACTCTCACACCTGGGAGCCAGGTGAGTCTGCTTACCCCCCAACCCCATTTTTAAAAAATGAGCAA
ACAGAGATTTGCCACGAAAAGGATTCTGGGGCGACCCACCGGGGTTAGGAGTGTCCCTGATGTTGGGAATAACCUTTTCAGATTTGTACCATCC
TGACCACAAAGTCAGGAGGCCTCCCCGGGATGAGTGAGGAATTTGGCTACCGGAACAGGAGATGAAGCCACACAATTCCTTCTTCCCTTTCTCA
GGTGGCCTGTCACCTCCTGGGTCTATAGATGTCATTTAGAATGGGTCCCTCAGACCCCGAAGGCCTTTGAAACAGCCTTACCAGGCAGGTCTCC
ATCCTGGCTCACTCTGGATGCCTCCCTTCAAAGTAACCGTTGGAACAGTGACCGCAGCCGAGACGCACATGACTAACCAAAATGTCTGCAGCCA
GTGGGAGACAAATGCTTGGCAAATCCCAGGGCTCTTTCACAACCCTCTTTTCTTCCAGGATCCAGAACTGGTGGGAACTGACTCCTGTTATCAC
TGGTTCTCACTGGTGTGCTTAGGCGATGCTAATCCAGTGCTGGCTACCTGCGCAGTGGGGGTTGGGGGAACGACCTCTGGGGTGACTCCCGTCT
GTTCTGACTTCTGGAGCTGTTGTGGGTTTACTGCCCACCGCAGAAGCCAAAGGAAACACTGCTCAAAACCACCAGAGGCAAGCAGCCTTTCAGT
TGGGTCACCTGCCCAACACCAGGGCTGTGAAGCTGACATTGCAGAGTGGGGGGTGGGGGTGGGGNNNNNNNNNNNNNNGNNNNNNCCTGACTCA
CATCCTGTTCTGTTCTCTGAGGCATCCGAGAGGGCATTACACCAGCTACCCATGACTGAGCTGCACTGGGAGGGCGCCCTGAGGATGACTCTGC
CTTCTGCCTTGCACAAAGAGGGGGAGTCTTCTAAGCAAAAACCCAGAACAGGCCCCTTGGGTTTATCCATTCTCAGAGCAGATTTTGCGCGTTA
AGAAGCAAGTGGCCCTGCATTGCATGGCCCACTGTTTATTTTCAATGGATCGAGGGTGAACGGCTTGTTGTTTTAGAGATGCCCGCCAAATACT
CAAGGAACAGAGCCCCGACTACAGCAGAGCCCCAACGTCCTCACCCACAAAACCCAGGCTGTGAGGTCAGGTGCCAGGGCCACCAAAGGCTCAA
GTGAACTAGGCTGATGTCCCCGCGTGTCCTTTCCTTGGACAGGGAGGGAGGGCACAAGGCTGAATCGGGATGCTTTGAGTTTTCGAAACAGGAA
CATTTATTTACTCTTCTGATTGGAGTCAGCTTCATCGACCACTTTAGATACGGCTACTTCTCTTTGCCTCGGGTGTGGGGCATTCTTTCCCCCT
CCTCATCCCATACGCAGTAGTTACTGGCGTTCTGGCTTTCCTTTCAAATATGTTTTCCGAACCAGAAATTTCCCTTCCAAAGCTGCCTCTTCTC
CAGGCCCTAAATGTTGGTGTTTTCATTTTCATTAAGTGACTGGCAGTCGACATATCTTTTCAGATCTCTTATTTGGTGGCTATGGCATCTCCAG
ATAATCTCCAATTTTTGAAGATTTCTAAGTAATAGTTCTGTTACCGGTTTTTGTTGTTGTTGTTTTGTTTTGTTTTTCGGGGGGAGGGGAGAGT
GTTGTTTGGTTTTAGTTTAAGTCCTTGTGGCCTAACGAAATACTTTGTATGATTATGCCTTCAAACCCAAGCAGTCTTCTGTGGCCTCAAGTGT
AGTCTGCCTTAGCCAGTCATCTGTGTGCCTCAGAAGAAAGGAGCGGCCTACCCGTGTTCTGTAGGCCAAACTGATACTGTCCAGGTCAGCCGAG
TCTGAATTTCTGCTTGCATGGTCTATCGGTGACTGGAAGAGGGTGCTAAAGTCTCCTGCCGTAAGGATGGGTTTCTCTGCTTTTCCTTGTATTG
CAGTTAGTGAGACCCTTTGTTCACGGGACACCTGTGAAGGAAAATGTATATTTGGTTTTCCGTACTATCATAACGTAACTTATCTCTTATCCCT
GAAAAGCTCCATCTTCAATCGGCTTTGTAGAAAACTTGATACAGCAACTCAGGCTTTCCTTGGATTGGTGTTAGCACAGCTTCTCCACCCTTTT
ATGCTTGCCCTATCTGAATCTACATTTAAATGAGCCATTTGTATGCGTCAGGTAGTTAAGTTTTATTTGTAGCTTGTTCTGACAACCTCTGTGG
TAGATTTAGACAATTCACCTTTATAGATACAGTTGCATTATGATCTGCCATGGTCTAACTTTTATGCCTGTTGCACCTGGTTCTTAGTCCCACC
CCCGGCTTTCCTGCCTCCTCTGAATGGAATCAAACATTTTATGTGACTTCATTTTAGCTTCTTTATTGCCTATCGGGTAGCAAAATGATTTTTT
TTTTTAAATAGTTTTGTGGGAGCTAGAGAGCTGGGTCAGACATTAAAGCACTGAATGCTCATCCAGTGGACCTGGGTTTGATTCCTAGTAACCA
CTAGTGGCTAAAGGCGGTCTGTTAACTCCAATCCCAGGGAAATCCAACACACGATTAAGGTCTCCCTAGGCACTACAAGCATGAGCTACACAGA
TATGATGCAAGTAATACAACACTCATATGCATTAAACAATAAATAAATAAATAAATAAATAAATAAATATTTTAAAATTGCTTCAATAATTGCT
GTAGAGTTTAAAAACTAAGCCCACAATCAAATAATGTTATTCCTTAATTGTTATATGTTACTTATATTTAAACTTTTGTTTACATTTTTAAATT
TAGTGTCTCTCTCTGTGTATATGTGTCTGCCTCTCCCTCTCCTCTTCCTCCTCCCTCCCTCCCTCCCTCCCCTCCCCTTCTCTCTGTGTGTGTG
TATGTGTGTTTGTGCGTGTATGTGTCTGTTTCTCTCTCTTTGTGTGTGTGTATGTGTGCATGTGTGTATATGGTGTGTATATGTGTCTGTCTCT
CTGTGTGTACATGTCTGTCTGTCTGTCTCTCTTTTATGTATGTGTCTGCTGTCTCTCTTTGTGTATATATGTATATGATTCTGTCTCTCTCTAT
CTATATGTCTGTTTGTCTCTATGTCTGTGTATGTATGTATGTATGTATGTAGGTATGTATGTATGTATGTGTATGTGCCTGTCTCTGTGTGTGT
GCTGTCTGTCTGTCTATCTCTCTCTCTCTGTCTCTGTGTGTGTACATGTGTATGTGCCTGTCTGTGTGTGTGTGTGTGTGTGTGTGTGTGTGTG
TGTCCTGTGCTGTACATATGGAGGTCAAAGGACAACTCACAAGAAGTAGTCGTTTCCTTCCACTATGTGCGTCCTGAGGATCAAGCTCGTGTAT
TGGACAAGCGCCACTCTGCTGAGATGTATCACTTCTTTCTTATCTGTCATTATTGCCAATTATTTTCTTGATCTTTATGCTGTAACTGTCCAAC
AAATTGTGGCTGTTATCACTTCTAACAGTTAACTTTCAGATCCGTTATGAATAGAAAATGGGTTTTATTTTATTTGATTCACTTCTTCTGGGAT
GACCTCCTTTTCACCCAAGATCTAAGTTTCTGGTCCATTTCXFTTTCTTTCTGTCTGAGGCCCTTTAAACATGTCTGTTGGTGATCATTCATTT
CCATTTCATCAGAAGATGTACTGCTTTTCCCTTTGCATTGGAAGAATAGTTTTGCTGGGTATAAAACTCTGGGTTGGTTTCACTTTGTTCCCTT
CAGACCTTTGGTGTGAGTTTTCTGTGATCGTCCAGTTGGGTTCAATGATTACAAAGCTATAGTTGACAAAGGCATGTAGTTCCCCCAGTGTCTT
TGTTACTGCCTCCCTTGTTGTCTTTGAGTTTCCCATGAACTCTGCGTTAGTCTATGCTTAAAGCTCTTTGGTTGGCAGTCTACCATAAGATGGG
AAACCATGAGAACTCAGTTGAGTTCCTGGGCCACAAACATGTTTCTCCTGGACAGTACTATAGCTCTCCAGACTTGTATTCTCATTATCAGTCT
ATATTCAGCCTCCAGCAACTTGTGACATCTCCATGTTGTACTTCCATGAGCTTCTGTTTCTAATCAACAGACCTTGGCTTGGTTTTACATGCTT
GCCTGCCTCCCAGGACTTGCGTGGTGCCCTGCTTTCCTGCTAGAATTCTGTGGGAGATACAGGAAAGTCACAGATTTTCTGTTCTACTTTTTTC
CTTGTTCTTAGGACAAGAATGGTAACTCCAAAACTCTTTACATTTTAGTGCGAAAAAAGGAAGAAGCAGGCCACTCTCGTATGAATTATTTGGC
AGCGTCATGGTGGACTCTAAAGATGTTTTGATGTTGGTACCTTTAAGACATTCTTTTCCTGAGTGAAATATGTCATGGTGCTTTGTACTGTCTC
CCTACCTTTCCCCTTATCTTTGACCTCCATTTCAAATGGAAGGGAATGGGGTTCAGGGATGTAAAAGGAGCCCCTCTTATCTATTATTTGTAAT
GGCCTGGTCATGTAATCTCATCAAAACCATTTGTCCTGAAAACAAAAACAACCAGCATACAGAAGGCTCAACTTTTCTGCTGCTTTTAAAAACA
GAAGCATGGATTGATTAAAACACCGAATTCCTGCTTTCTCTGTGTCACGAAATTCCAGACTCCTTACTCATTCAGCATCTAATCCCCAGAGCCT
ACCTCTTCTCAAGATACTCAGCTCCAAGCACTCCAGTAGTACCTCAGTCCCACCCCAGTTCAAGTGTGTGTTGCCTTACCAGATAAGTCTCTTG
GCAGGACTGAATTTCCTTTAGCAATGCAATCATGGAAATCACTCAGATTAATTAACCATTTTTGATGGCCACATATTGTGACAGGTCTTTTTCC
TGTTGCTTGCTTGTTTCTTGATGTTACTCTTGCTAGTCAGAAGAATGTAAGTCTTGCCTGAAGCTAATTTATGGTGGAGGCAAGAGTCAACCCC
AAATCTGGTTGACTTACATCCTAATGCTTTCCTGCCCTGCTCCTCCCCTCTGCAGAACATAACCATCTCACAAAAGTCACTGCTGCCCTTGCCT
CTACCTTGGAAGCAGACATTTGCAAGTCCAAATTAGTCATGTGATTTAGCAGCCAGCCTCCATTCAACCTTTACATCATCCCGTGTCTGTAAGT
CTTACAAAGTAAAGACCATAAAACCATTTCCTAGTGAATCCCTACCACTAGGGCCTGGGTGAACCAAAACTATTTGTGGGAACATGAATGAGGG
AACAAGTAAATAATTTCCCCAGCCTATGAATATACAAGTGTTTGAATGAGTGAATGGGTGAATGAATGCATGCATGAATGAATGCATGAATGAA
TGAATGAATGAATGAATGAAGCAACTCTTAGTGTGCTGACATGTGCATGAGGAAGGAACCTTTCCAAGCATCACAAAGTTAGAAGCCTTTCCCT
GATGAGCCAAGGCAGGGGCCATGTGGCTCCTAGTCACCACTATCACTGGCCACATTTCCTGTGCCTGGCACACGGTATATGTTTAACCAACTCT
TGTTCACTTGGATGAATTGGCTTACCAGAGGCAACTCCTTCAAGACTAGTACACCTGCACTTTGCCGAATGCCTAACGGTGACTCTCTCAGCCT
GTGGCCTGTTCTGGATAGCTCCCAGTTTAATCCCTCCTTGTGGCATTTCCCAGAAGGCACACAGTTACGATTTTAAGTACCTACACCTCGCCTC
CCATCCCCTACACGACTCTTCCCTGACTCTAGCAACATATAGCAAAGATGAGTTGGACTAATTTGAACGGGGAGCCTCCCCTGTTGCCACTGGA
TGGCTTTAGAGATCATCTCTCTGCCGAGAGAGAAGTATGGACGTTTGGTCTAGGGCAACTGGCCCTCTCAGCAAACCGATGGGCCTAGAGCTCC
TTTTTCTGATAGGCAATCAACAATATGGTTGTTATGATTCTAACTTTCCTGTGTTCCTAGCCCCACCTCCCCTCCCATCCTAACAACGATAGAA
CCTCTCCCACAAACGCAGAATGCACTCTATTTCCCTGCCTCTTGTGGTTTCTTTTTGTTTTTAATAGAGCATTAAAAAGCTGAAAACAACCCAA
ATTACAAGAAGAGGCTCACCACCACCACCTACTGGCCCACCCCTGGCAAAGGACCCAAGTAGGTTCCTGGTGCTGGCGGAAATGATGGTTGATG
GGCCGTGGCGTCAGTGACCCACTCAATACTGTGGTGGGAAGTAAGATGATGCCAAATCTATAACCTGTGTTTAAAAAAAAAAAAAAGAAGAAGG
CGCAGATTCTGAGCCTGAATCATAAAGGCCGTTCCACCGAAGCGAGGCTGTGAAATATTACGCAACGTTCGCACGGTTTAATTACGAGTTTAGA
AAGCCCTAGCCTGAGTTTTTCACTACAGTAAAACATGACAGAGCATGAACCCTGGCACCACACTTTATGCTTAGTGTGGTTTTAGACACAGGGC
TGGGGTGCTAGGCTGTCTACACAACAAGGCATGGATTACAATACCGCAATTCACACCCCGGGGGCCTTCATGACAATTACAGAACCTCCTTTGT
TTGCTTTTTAAGTGATTAAAATATTTGAATAGTATATGTCCATTTGCTAAGTGTGTATATTGAAAGCACTGGCATGTCTTGTGGAAAACATCTG
AGAGTGTGAGCTTTGCGCTCCCCGCACACACACAATGGAGCAAGATCCTTACACTGATGTGGGATGCAGGACGGTGTTGTTTCTACACACCTCC
GGGCCTCAGGAACAGTCGGCTGTAAAACCGCACAAGGCTCCACCTGCATCCCACCACTTGTCTGATGAGTTTAGTGTGTGCCAGCTCCCTTGTC
TGTGCCGTCCCTTCAGACAAACTCTCCACCATTGGGAGGATGGCAGGAGATCTCTTCACCGGGAGATTCTAAGTTAAAATGTTGGCCCAGGAAC
AAGCAGGTCTGTGGTCCTTACATTGTCGGGTTTGTGGTTATCGTTGTGTTCTATTTTCCCGGACATTAAAACCAAACTGAGTGAGACAAAGGAA
CAAGGCTTATTGAGCCCCGAAGTCAAGTGCACAAGAGACATTTGCTTCAACTCCCGGCTCTGTCCCTGCTCTTTCTTGGTGACAATCTATAAAA
TACATTCTCAAAGTTTTGCCCTTCAAAAACACCACTGGCCCCAAGAGTCCATGCTACCTCGTTTGGTCTGTTGTGAAGCAGCCTCATTAGTGTG
CCTGTGTTTTGGTCGTTCACCATCTTTGCACTGTCTGTCTGTCTGTATCTCTCTCTCTCTCTCTAGTCTCTCTCTCTCTACTCTCTCTCTCTCT
AGTCTCTCTCTCTCCCTCTCTCTCTCTCTCTCTCTCTGTGTACGGGGGAGAGTTATCTTTTTACTATCGCTCTCGCTATTGTTTCTATGGCATT
TGAGGAATGGCATTTGCTGAAGTAGAAAACATACACATGCCCGTGGACATTTCCTCAGGGTCCCCCACGCTCCAGACCCCCTTACATCCTTTGT
GCTTCCTGAACAAGGTCGCCATCTGAGCCGATACAATGTTTCTGGTGATGATAACTCTAATTGCTGGCCACGATGAATGTGTTATCCTGAATGT
GTCACTTAGAGTGATCCAGTACTACCAGAGTCCATTCCCAACAGTATTAGTGTGAAAAAAAAAATGTATATATATGCTATATATATTATAGATA
GGTAGATAGATAGATAGATAGATAGATAGATAGATAGATAGATAGATGGAAGGAAGAGTTAGCAAATAAAACTCCCCCTAAATCCATGAACTGT
ACTTCAGTATCTTTAATGGGCCTTCATACAACAGACTCATCAAATACTTGGGCTCTAGTGGTCTCATTAGACACAAGTGAACAAGCTGTGTAAA
CACATGTATAATCATTTAAATAATGGTTAATAGCTATCATGATGAGAACTTTTCGTCTTAAAACCCCTGCCCCACCCCATCCATGACCAACATC
CTTGTCTCCCTGCAGATGCCAGGCAGATCCAGCCATCCCCACCGTGGTCCTATGACCAGTCCTACCAGTACCTGGGATCCATCACCTCTTCCTC
TGTCCACCCAGCGACACCCATTTCACCCGGCCGTGCCAGCGGCATGACCAGCCTCTCTGCAGAACTTTCCAGTCGACTCTCAAGTAAGCCCTGT
GAAAACACACCTGCAGCGGGGCTCGAGCGGAGCATTCGGAGGACCGCGGGAGACTCGGGGAGACACAGAAAGACACAGGAAAGGCGGGACATGG
CTAATGGACCAGAAAAGGGTTCAAAGACCAAATTTCCTGCCTCTCTGCGTGTCCAGTGCCTGAGCACTGATCTTTCTTAACATGCTAAGAGATG
CCAGATGAGGAGGCAGCAATGCCTTAGTCCATCCCTTTGGGATTTGGGGTTTGCTCATATCTGGTGAGAAACGTTGGTGGCCAATGGTTACTAT
TCTGATAAGTGGAAGTGGCCTAAAGATTTTTGTACCTTTGGCCAAGAAATTAACATCCCAACTGAATTCTAGACCGAGGCTATTCTTTCCACAA
GTGGCCAGTTTTTATGGCTCCTGCATACACCACAGTGGGGTACATACTGGAATAGGACACAAATACGCCCCAAGATAAACATTCCCAGCTAAGA
GCTGTTCAGACTGAGGCTAGGAACCAGGTAGAAAGCTCAGGTGTCATGGTTAGCATAATTGTTGTTAATACGAGCTCATTACATATTAGTCATT
TAACCTATGAGCCCTTTGGGCTTGGCACACTCACCTTCGCTCCTGAGCAGCGAAATCAGAGGTACTGGGGGAAAAAAATTCCTTTGTTGAAGGC
AAAAGAAGAAACTTACTGTTTGGGGCTTTGAAATTGGAGCCGAAGCAAACTGTGAAATACTTCAGAGAGCTTCGTCCTTTAGAGGCCCGTGAAT
GCACTCCCATGTGTGCCGGGGGAGGGGCAGGCTTTGCTTTCGGACTCTTGAAGGCAGATTAGAATAGATGAGGGGGCAGGCAGAGGAGTTGGGG
CTGCTGCTTTAAATGTTCTCAAAGACTACATTTCCAGACAGCCACGATTGCTTAAGACTCCTCCAGCGGGTCAGCTGCTCCCTCACAATCACCC
TCAATTCACCCCGCCTTTCCCCAGCAACCATACTGACATTTGGGCTGTATCATCTTTTGTTGTGGGAACAGGGGACTGGCCTATGTGCCAAACA
ATAGATAGATGACCCTTGACCTCCAGATGTCAGTAGTAAATGGCCCTGTCTGTCCATCAGAAATATCTCCAAGGCTGGGGACCAGCTCAGTGGG
CAAAGCCCTTATCTTGCAAACATGAGGACCAGGTATTAAGGCAAAGTGCAGTGGCACAGGCTGCTAGTACCATCACTAGGACATGCAGACATGT
CAGTATCCACACTAGTTGAATTAGAAACCTTCACGCTTGGGAGAGACCCTGCCTCAAAAAGCCAAGTTAGATGGCTGCTGAGGAACACACACAC
GCACACGCATGAGTCTGCACTCGAGGGTACTCATCAATGAACACTCAGAATGCTCCCACTCACCCCTTACAAACACAAACCCACAGAAAATGTT
GCTGGACATTGTCACTTGTCTCAGCTGGGGGCATTGAGAACTTACTAGCTTCAACTAGGCTTGGTGCTGGACTGAAATGCCAAAGCCAGNNNNN
NNNNNNNNNNNNNAGAGAGAGAGAGAGAGAGAGAGAGAGAGAGAGAGTTTGCCACGACCCAAAAGTGACCCTCTGCTGCCCAGTAGGGAAGACC
AGAGAATGACAAACAACTGGCATAGAGTGAGCCTCAACTTGGCAAAACTCCCTCTAGAAACCAAGCCCCGAGCACAGGCCTCAAAAGCCTGTAA
TGTGTTCTCATAAACCTGGGCCAGAGAGGGTAGGTAGGGTCCCTGAAAGGAAGAGGCTGTCAGGCCCACAGCTTGTCAGCCCAACTTGACCCCT
CTGTGGCTGATGTGCCTTCTAATCCCACCAGTGACAGCTTCAAGTTCTGGGCTTTCAGCCTCCCCCAGCCCGACCTGCCTAGCTGCTCAGCACA
CACACATGCGTTCTCAGCAGACGGGCCCTGTCTGTCTCTGTCCCTTGCAGACAGACAGACTCCACACCTTGTCTGAAAGGAGCTGCCACGTGAA
GGTTTATCTTTCCTCCTCATTTCTCTTCTTCCTTTATTTTACCCTCTTCCTTTTCTTTTGGTTAATTAATGTCTAGCTGCCAACCTGGGCGACA
GACAGCGCATACTAATCTGAGTCACGCAGGAGACCCTTTTAAGGCGGTCATTATCTGCTCAGGTTCTTTAATCCTTTGGGGGCAGTGGGAGGGG
CCTGGGCTGGGACCTCTTTCATCCACATCAAGCTTTAGAGCCAACACTCTGCGCTCTGTCAAGGTAGAGACTTAGCACCATAGAGGGAGAGACA
CTGCCAGTGAAAGTGGCTGAACCTTCTAATCCTCACACTTCCTACTTTTAAAGTCTCCCCATGAATGGGAGAGGGGGCAGTTGTCACCTGAATT
GAGTGGGTGGGGGCGGCGTGCACCGGACTAGCCAAAAGAATAGAGGAGCCGTAGTTGGGTGATGTATAAATTCTGTGTGACTGCTGATGTGTAA
GGAAAGAGGTTAGACAGGATTTAAAAATGAAAACACGGCGAGCTGGTGAGGTAGCTCAGTGGTTAGGAACCTTTGTTGCTCTTCCCAGGCCCCC
CACATTAGCCCGCTCACAGCTGCCCATAACTCCAACTCGAGGGCATCTCTCTCCTTCCTCCAGTGTCTGCAAGCACACACACACACACACACAC
ACACACACACACACACACACACACACATGCGCGCGAGCACCTAACTAAAAATAAAATAGCTGTAAATGAGAACACGTATTCGAATAGAGCACTT
GAATTTGAACATATCTAAAAGGCCCATGTCTTTTTTATTACAGAGCGTATACAAAGCGAGACAGGGAGGGATGGAAGCAGAAGGGGAGGAGGGA
GAGAGAACCTGACCTCTCGTAGAAAGACGCCACACTATCTCAGATCTTCCACATACTCTCCCCCCAGCAGCTGAGACTGTAACATTACTCCTGC
GCTTATGCCCATGACAAATCACGGACTCACTCGQGATTCCGGTAACGTGGTACCATTACCACATTACCATGTAAGAACTGCGACAGACTGGCAG
AGCATGTTCAGACACGGCTCTGAAACGTGGTGACCTCTCCTTCATTTTTTTCCAGTGGATTCACCTTTTTTGTCTCAGGACAAATGAAGCAAGA
GGGATAATGGQCAGAGTCACTTGTTTGTGTCCAGTGTATTGCATCAGATGACAACACGCCGATTGTGTGTGTGCCTAATGCTCTCCCTCCTGCC
TCGGCTTCTCCCTAGACTGTATAAATCTAACTGGAAAAAAAAAATGGAGTAGAGCTTCCTGTTAACTCTCAAGCATGTCATGGCTCTCTGTAAG
CAAAACAAGTGACACAGAGTTTCCTCATAGAGGTCCCCGGTGAGGGCGCAGAGATGAAC
MOUSE SEQUENCE—mRNA
CTCATTCATAGAGCCAGCGGGCCCGGGCAGGACGGGCGCCCCGCGGCCGGACCCAGCCAGGGCACCACGCTGCCCGGCCCCGCGGCTGGCACTT
CCATCCTGAGCTCCCCGAGACGCAAGAAAGTCACCCTTTGCGGCTCTTTCTAGATCTGCGTTGGACCTTCTTGTTTTCCTTTCCTTTCCTTTTT
TTTTTTTTTTAATTTTTATTTTTATTTTCCCTTTCTTACTTTTGAATGAACCGGCTTCTAGGCTTTGGATGTTTCAACTTCTAGGCTGTCAACT
TTTCTCCGATTGTTTCTTTTGACAACTGCGGGAGAAAGTGTTCCGGAGTTCCACGCCAGCGGTGAAGTTGCGTCTGCCTGGCAGGGTGCGTGGG
AGTGAGCGTGTGTAACTCGAGCCTACTGGTCTACGGTGCCTCCTCCCAGGCCCGCTCCACCTGCTGCGGTTACACGGTGCTCGCTCGTGGGTAT
TTGTGCGCCCCAGCAGAAAACCAGCCACCGCCGGGCGACGTTGGAGGAAGATCATCTTGCCAGACTCTGGGCTGACTCACTCAACCAATGTAGA
CAGTGGCTGACTTCCCAAGAGTGCGAGTCTGCCTGTGTGTGTGACCCAACTGCGGCTCAACTCCCACCAAACCACAGGATCAGCCACAAACTTA
ACCAGCGTTCCCAAACCAGAGTCCTCAGCTGTGGGAAGCTCTACCCTGAGTGTCATCGAGTTTGACTTTTTTTTTTTTAAGATTAGCTACAGAT
CTTTGTAAAGCACTGACCATTTATTTGGCCAGAATTTATTGAGTAATGACAAGTGATCCTATCCAGCCTTCCAACTACTGAAGCTGATTTTCAA
GGCTACTTAAAAAAAAAAAAACTGCAGCAAACATTAATGGATTTCTGTTGTGTTTAAATTCTCTACAGATTGTATTGTAAATATTTTATGAAGT
AGATCATATGTATATATTTATATATACGAGCACATACATTAGTAGCACAATCTTTTAAAGTTACGGCTCTTGCTTTTGAGAAACGAAGTGAGTT
TTTCATGGTAAGAGGGGCCCTCTGTATGGAAGACACTCCTAAGTTTTGTATTTTGTTGAGACTCTAAACAAGACTGACCCGCAAGAAAGAAAAA
CAAACTGACAGGCAACTAACTGGTGGAACTTCCAAATCTAGTTTTTGCTGGTCATTTTTTGGTTGTTGTTGTTCTTCTGCGCATAAATATTTTA
GGACGCGTATGGGAATTTTGCTCCGGGACCGTTTGTAATAGCCAAAGACTGAACTTCAACTCTCAAAGCGAGGCTCTGTTGGGCATTTGACTTT
GAATTCTGGTTGCCCACTTTTTAACGCACTAAGCGGCCAGTTGCTAACCCTGCTTGGGTGTGAGGCCGATCCGTGTAACTAGATCTCAGTCCGG
GACTGATGGTAATTTAGTCTCCTACCCCCACAACCCTCCGGTAGTAATAAAGGCTTCTGAACTTGTATGTTGGTCTCCCGGGAGCAGCTTGCAG
AAGATCCGAGCCCCTGTCGCCGTCTAGTAGGAGCTGTTTTCAGGGTCCTTACTCAATCGGCTTGTTGTGATGCGTATCCCCGTAGATGCCAGCA
CGAGCCGCCGCTTCACGCCGCCTTCCACCGCGCTGAGCCCCGGCAAGATGAGCGAGGCGCTGCCGCTGGGCGCCCCGGATGGCCGCCCGGCCCT
GGCCAGCAAGCTGAGGAGCGGCGACCGCAGCATGGTGGAGGTACTAGCTGACCACCCTGGCGAGCTAGTCCGCACCGACAGCCCCAACTTCCTC
TGCTCCCTCCTACCCACTCACTGGCGCTGCAACAACACCCTCCCCATCGCTTTCAAGGTCGTCCCACTGGGGGACGTCCCGGATGGCACTCTGG
TCACCGTCATGGCAGGCAACGATGAAAACTACTCGGCAGAACTGAGAAATGCTACCGCGGCCATGAAGAACCAGGTAGCGAGATTCAACGACCT
CAGGTTTGTCGGGCGGAGCCGTACAGGCAAGAGCTTCACTCTGACCATCACCGTCTTTACAAATCCGCCACAAGTTGCCACCTACCATAGAGCC
ATCAAAATCACAGTGGACGGCCCCCGAGAACCCCGAAATGCCAGGCAGATCCAGCCATCCCCACCGTGGTCCTATGACCAGTCCTACCAGTACC
TGGGATCCATCACCTCTTCCTCTGTCCACCCAGCGACACCCATTTCACCCGGCCGTGCCAGCGGCATGACCAGCCTCTCTGCAGAACTTTCCAG
TCGACTCTCAACGGCTCCGGACCTGACCGCCTTCGGCGACCCACGCCAGTTCCCTACTCTGCCGTCCATCTCCGACCCGCGCATGCACTACCCA
GGCGCCTTCACCTACTCGCCGCCCGTCACGTCGGGCATCGGCATCGGCATGTCAGCCATCAGCTCGGCCTCTCGCTACCACACCTACCTGCCGC
CGCCCTACCCCGCCTCATCACAGGCGCAGGCCGGGCCCTTCCAGACCGGCTCGCCCTCCTACCATCTATACTACGGCGCCTCGGCCGGTTCCTA
CCAGTTCTCCATGGTGGGCGGAGAGAGATCGCCCCCCCGCATCCTGCCGCCCTGCACCAACGCATCCACCGGCGCCGCGCTGCTCAACCCCAGC
CTCCCCAGCCAGAGCGACGTGGTGGAGACCGAGGGCAGCCATAGCAACTCGCCCACCAACATGCCCCCCGCGCGCCTGGAGGAGGCCGTGTGGC
GGCCCTACTGAGCTGAGCGCCATCGCCATCGAGGGACTGGGCCTGCCGTCCATGCACAGACCCCGCCAGGAGGGCCCTTGGAGGCCACCAGGAA
GAATCCCGGAGGGAAACTGTGAATGCTTCTGATTTAGCAATGCTGTGAATAAAAGAAAGATTTTATACCCTTGACTTCACTTTTTAACCACGTT
GTTTATTCCAAAGAGTGTGGAATGTTTTCGGTTCGGGGTGGGGAAGACGCAGCCCATCCTGTTTGGCATCTATTTCTTATTTCGGAGTTTTCTT
TTCCGCACCTTATCGATTGCAAAAATGCCTGTTTGCATCTGGGTGGTCATTTATTTTTAAGTGTGTATAGATTTGAGCTTGCTTTTTTTTCTTC
CTTTGACCAACTCAAAGAAATAAAATTCCCTTCTCTGTAAGGTTTATTTAACTTTTAGACTTTCATGTAGCTTTGGGGTTTTATTTGTGTTTGG
TTTTTGTTTTTGTTTTTATTTTTAAAGAGACAGCTACAGCTTTGGGTCATTTTTTAACTACTGTATTTCCACAAAGAAATCCCTAGATATTTAT
GTATCTTGATGTTTGAACATTTACATATGTGTTGATACTTTTTTAATTATTTAAATGTACTTATATTAAGAAAGATATCAAGTACTACATTTTT
CTTTATAATAGCCAAAGTTAAATATTATTGCGTTGAAGATGTCTGGAAAAAAAAGAGATCGCTTGGTTAACTAGAAATATTGTTTACATTAAAC
TCCCTTTATGTTATTCAAACAAGTTGGTAGGTAACGCAGCAATGTTTTTAATTGGATTGTAGACACTGAGGGTCACTCCAAGGTCAGAAGTACA
AAATTTTCTGCTAGGCTCAACAAATAGTCTCATACCTGGCTCCTTCCCTTCAAAAAGAGAGGCAAACTCTGTCCTGAAAGGGTTCAGAGAGGTG
CCAAGGATTTGCTCTGAAGAGGATTTCATTTTGGCCTGGAGATATACTTGCCCCAAGGCCTCCTCATTCTGGCATGCTTTATCACAGAGCTCAA
CCAAGTAAGCTGTTGGTCAGGGGTTTACTTACATAGTATTTACATAGACCCAAACCACTGAATGTGATTTTTAAATTGCCTTCCATTAATAGTA
CCCGTTCATTGATGAAAACCAAAACTTGAGGCTGTACCCCAAAGATCCAAATAGAAGAGTTAAGACCAGGTGTCTTTGAGGCCTAAAGGCTGAG
TTTTAAGAGAGTGTACCCCAAAAGTCTGAAGGAGCCGGTTTCCTTCTCCCAGTCTTAGTGGAATCAGTCATGGGAGGCAGATGCCACGCCCACC
TGTGCAGGATGCTCCTCAGAAGCTGCCCCTTCACCAGCATCTTCTCCCACCAGGCCGAGCCCCTGACCTTTGGGGTGCATCAGTGTGATAGATC
CTGGTCTCTGCAGTCCGCCATGGCTACGGTTCAGATGTGCATCGTGTCACTGTAAATGTAATGGTACTGTTGTTACAGTGGAGGACTTGGTCAA
AATCCAGTTGTTCTACAACGTATGAAGCCTAACCGCTGGTTCTGACATACATGTGCTCAAAATGA
MOUSE SEQUENCE—CODING
ATGCGTATCCCCGTAGATGCCAGCACGACCCGCCGCTTCACGCCGCCTTCCACCGCGCTGAGCCCCGGCAAGATGAGCGAGGCGCTGCCGCTGG
GCGCCCCGGATGGCGGCCCGGCCCTGGCCAGCAAGCTGAGGAGCGGCGACCGCAGCATGGTGGAGGTACTAGCTGACCACCCTGGCGAGCTAGT
GCGCACCGACAGCCCCAACTTCCTCTGCTCCGTGCTACCCACTCACTGGCGCTGCAACAAGACCCTGCCCATCGCTTTCAAGGTGGTGGCACTG
GGGGACGTCCCGGATGGCACTCTGGTCACCGTCATGGCAGGCAACGATGAAAACTACTCGGCAGAACTGAGAAATGCTACCGCGGCCATGAAGA
ACCAGGTAGCGAGATTCAACGACCTCAGGTTTGTCGGGCGGAGCGGTAGAGGCAAGAGCTTCACTCTGACCATCACCGTCTTTACAAATCCGCC
ACAAGTTGCCACCTACCATAGAGCCATCAAAATCACAGTGGACGGCCCCCGAGAACCCCGAAATGCCAGGCAGATCCAGCCATCCCCACCGTGG
TCCTATGACCAGTCCTACCAGTACCTGGGATCCATCACCTCTTCCTCTGTCCACCCAGCGACACCCATTTCACCCGGCCGTCCCAGCGGCATGA
CCAGCCTCTCTGCAGAACTTTCCAGTCGACTCTCAACGGCTCCGGACCTGACCGCCTTCGGCGACCCACGCCAGTTCCCTACTCTGCCGTCCAT
CTCCGACCCGCGCATGCACTACCCAGGCGCCTTCACCTACTCGCCGCCCGTCACGTCGGGCATCGGCATCGGCATGTCAGCCATGAGCTCGGCC
TCTCGCTACCACACCTACCTGCCGCCGCCCTACCCCGGCTCATCACAGGCGCAGGCCGGGCCCTTCCAGACCGGCTCCCCCTCCTACCATCTAT
ACTACGGCGCCTCCGCCGGTTCCTACCAGTTCTCCATGGTGGGCGGAGAGAGATCGCCCCCGCGCATCCTGCCGCCCTGCACCAACGCATCCAC
CGGCGCCGCCCTGCTCAACCCCAGCCTCCCCAGCCACAGCGACGTGGTGGAGACCGACCCCAGCCATAGCAACTCGCCCACCAACATGCCCCCC
GCGCGCCTGGAGCAGGCCGTGTGGCGGCCCTACTCA
HUMAN SEQUENCE—GENOMIC
AATCCACTGGTGCCTTTGTGTGGGAATGAGAGTGAATCTAGAGAGATGAAGACAATTATCTGGTCGGCTGTTAGGAGGCTCAATAAAAAACACC
CCTGCTCTCCGGTGATCCACAATCAACAGTGAGATGACAAAGGAGTCAAGCAGATGGAGATAAAAATACCAGCTTGGTCACTGATAGAACTTTT
GGAGAGCATGGCTTAGGTATGTGGCTTCCTGCTACTGAGCTCCAGAAATAGGGAGACTCACTCACCAATACCCAAGCAGTGTCTCCCCAAAAGA
ATAAGGAGAGGGAAAGAGCATGCCCAGGGTACTTGTTTTCTTATTGCTGTAAAACGAGCAAATTGCCAAGCTTAGCACCTTAAACAAATTTAGC
CCACAGTTCTGCAGGTCGGAAATCTGGTGGTGCTGTACTAGATTCTCTGCTCACAGCTTCATAAGGCCAAAAATCCAGGGGTTGGATAACCTGA
GCCCTTACTTGAAGGCTTAGGGAGATAATTCACCTCCAAGCTCATGCAGGTAGTTGGCAGAATGCAATTCCATGGGGCTGTAGCATAGTGGCCC
TCGTTTCCTTGCTGGCTGTCAATCAGGGGTCACCCTCGGCATCTAGAGACTGCTCTCTAGTTCTTGCAAGTGCCTGCTCTGTCTTCTAGCTACA
ATGCTGACTTCACTTCTGCCCCAGCCTGAGAAAACACTGTGTTTGTAATGGTCTCCTGTGATTAGATTAGGGCTACCAGGAAAATCTCTCTGTT
GCATGAGTCAAAGCCAACTGATTAGTAACCTTAATTACATATGCAAAAAAAAATCTCTTTTTTTCCATGTAACATAACCAGAGCATGACAACTC
ATCATATTCCCAGCCCCAGGGATTAGGGTGGAAAGTTTGAGGGGTTCATTTTATGCTTCTGCCTTCTACAACCAGTTTCTGCTTCCAACGCTAC
TAACTAGGTAATTCAATCTTTTATGGTGGAGGAGGAATGAGGGTGTAAAAAGAGGTCAAGAGTTTTACACTAAGGAATGTTGTTCCACATGGAA
ACCTGAAGCCAGGGAAGGGGTGGTTCCACCACTTCCTCTCACCCGCCCCACCACCCAATATATTTAAATAGCTATAACCCAAAATATATTATAA
TTCAAGTCACATAGGAGATGTGGAAATGATGATAAAGCATGTGATTGGTCTTTAAGAGGCAATTTCACCTGAGGCAGAAGGATCTGCCCCCCTC
CAAGATGAGGACAGGAAGGGTAGCTCTTAATCCTGCAGAACTGTGCTAAAGACCTTGCATTGAATTCCAGATGCCCAGGAGAACACAGCAGAAC
TATGGTGCAGGAGACTAATCTGGAAGTACTGTATACAATTAGCAGGAACCCCCTTTTTCAGAGATCTTTGTAAGAAATCTTCAGAGAGGTAATC
AGAACTTGAACCAGAATGATGTCAATAGGAATTGAAAGGTGGGCGCCGAATTGTTATCAACCAAAGAAATCAAAAAGAGATCTTGGCAACAGAC
TGGAAATCAGCGCAAAGATCAATCCATGGATCCAGTCTCGAACTGGGAGAATGATGGTCCACTGGGGAAAACAAGAGGAAGACCTGGGTTGAAG
TGGAAACTGATGAATTTTATTTTCAGTAAAATACACATGGCAAACACTAATTTTCTCAGGTTGCTTCCATCTTACACACATGCACACATATATA
TACACAAACACCTCTCAACACTGGTCTTTCTCTCCTCCTGCTACCTCACCAGTTTCCCTCCCTCCTCCAACAAATTTCATTCTCTCCTTCCCTA
CATCCCATTCGCTTTCTACTCTCTGCTCCATTTCTGCTGCTGCCACTCAACTGAAGCTCCTTTGGCAAAGGTTACATGGCACTCTCTGTAGACA
AATCCAGTGGAATATTTTCATATTTTCAGTCCTTCTTTTCATGGCCCCTTTGTGGCCTTTGGGCCATGGTTTTCCTCCAAAGCTGGCATTTTCC
TTTATTCTTTTCTTTTCTTTTTATGAGACAGAGTCTCATCCTGTCTCCTAGGCTGGAGTGCAATGGCACAATCTTGGCTCACTGCAACCTCCGC
CTCCTGGGTTCTAGTGATTCTCCTGCCTCAGCCTCCTGAATAGCTGGGATTACAGGTGCCCACCACCATGTCTGGCTAATTTTTGTATTTTTAG
TAGAGACAGGGTTTTACCATGTTGGTCAGGCTGGTCTCGAACTCCTGACCTTAGGTGATCCACCTGCCTTGGCCTCCCAAAGTGCTGGGATTAC
AGGCGTTAGCCACCATGCCCGGCTGCATTTTCCTTTACTCTTATTACTTATTCTCTCCTCTTTCTTTTCCTAGCTCCATCTCCTTGGTTGCTCC
CTCTTCTGCTCACCCTGGAATGTTGATAATCCCAGAACCTGAGAATGGTCTCTTCTCACTGGAAACAGTCTCTGAAACTAGCATAGAGTTTGAT
TGTACCCTAGATACAATCTGAAACTACAATAGATATTCTAAGGATGTTCAGCTCATTATTTCTACATGAAACCTCTCCCCAGAATTCCTGTTTT
CCCCACCAGCACCTCCTATTCTGTTCTGCGCATCCAAAGCTCAGAGAGACATCACGATGTCTCTCTGTAAACCCGCTGTTTCTGGTCTTCTCTA
CTACTATTAAAGGACAGATGCTGGGGAGACTCCTCCCCCTCCCTCACCTCAATCCCCTCTTCCATCTCCATTGCTTAATCCAGATCCTCATGAC
CCCTTGTCTGACAACTGCTATAGCTCCCCACTCCTCATCTCTACTCCCTCCAACTCACCGAGCTGTTGTTGACAGACTTATTTTTCTAATATAT
ATATTAATTTTGTTATTTCCCTGTTTGGCTTTGACAGATCAATTTTCCCGCATGTTGAAGTTCACATTTCCTCAGGTTGCTTCTGCTTCCAATG
ACCTATCTACGTCTATGTATTTTTGAACTGCTGCACTCCCTCCCCCACTCCCCAACCTGGTCTCAAACCTATGCCCCAACTGTACTGGAACTGA
CACACTGATGTGTTTCATAAACTCACGCCCTTATTCATGATGCTTATTCAGCCCAGAGCACCATTTCCCCACCCCACCGTCACCTCCCTAAACC
CCTCCTCTAGCTCTTTGAACCACCTAAAAATCTACCCAACCTCCAAAAGTCAAGTCAAAAATCACCTCTGCTAGGAAGGAAGTCTTTCCTGAGC
CCACCAGGGGCTGTTGGCCCCTCTTCCTCTTATATTCCCTCCCACCAGTTCCTGGGACCCTGGACATGTGTCCACCAACAGCCATGACACTTCT
TTACTCTCATCTGTTTGGTGTTTGTCATCAGTGAGCCTAGCACCTTGCCTGGGACAGAGGAAGCATTCAAGGAATGCTGAAGGCAGGTGAATCA
AATACCTTGTATCTAGGAAAGTCCTAAAGATGCCCTTTTAAACTAGTGGAGGTGGGAAAAGACTAAAACGCCATTTTATATTATTATCAATATA
AAAGCTTCTCAGCCAAATGGACCTTGGCTTTCTCCTCAAACTCTGTGCATCACAGAAATGACATTAAAATCTGACTCCTAATGATATGAGGTTT
GCATGGTGAAGAATTTCCAAGATATTTTCTGATCTAGACCCTGAGACGGGGGGGATAAAAGTGAAAGATGAGATCAGAGTTAATTTTTTAGAAC
TCTACCAAGGATAGCCACAAAAATAATCATGATCCAAAAGGAACATAGCTTACCATGCATGTGGACCTATATGTAGATATGAATATGTGTGTGT
GTGTGTGTGTGTGTGTGTGTGTGTGTGTGTGTGTGTGTGTTTCTATGTAGGTCAAGAGTGATGTGAATTTCGCCAAGTTACCTGAACTCTCCAA
GGGTGAGGTTCCTTATCTGTAAAATGGGGGTAGCAATGATGTTCTTGTGAGGATTAAATCAGGCAAAGTATATAAAAGCACTTCACCCATTGCC
TGGCATATAATTACATTCAATGGGGGATGTAGGGAAGAGAGAGACAGAGAGAGAGCATGCAAGCTAGCACACTGGAGGATGGGCTACCAGATTC
ATTGTTCACTGTCTTAAACACACGCAACCTCCCCTCTCTTGCAGTTTGGGTTCTGGCATCTTTAACAGTTTCATGGGCAAATCACTTCAAACCT
CCACAGGTGCTACTTCACCTGGGCAGCAGTTGCTCACCCCCTACCCCTTCCCCCTCCCCAATTTAACCACACTTGCTGTGTTCTCAAGGCTGCT
GATACACAACAAACTAAGAAAGTTTTTAATAGTTACCAATCACAGTTTTCAAAATCCACTCATTTTTGCCCTTGTTTTTGGGTTTAAAAAATCC
TGGCATTTTCCTCAGAATTGCTGAACTATTAGGTGTGTAGATGAACTTGTGTTTTCTTGCTCTCCTACTCCAATTTCTTAGACTCAGACATGGA
AAAGCTTCTCTCGGTCTCAAGACATAATTTCCAGGGTGCGTGGTTCCGCTGGGTGTGCTGTCTGGTCAGTCTTACCGTTGTGGATTCCACACCC
CCACCCCACTGCCCCAACACCTATTAAAGCTGAGCTGACGTAACTTCAGGATACATTTATTATTTCTTACCTTATCATTCACAATTAGTGCAAA
TGATTAGTTCTCTGCCTTGCTCTTTAAAAAGAAAAGTCTTCATGGAGTCATTTTATCTACTTGTGAACCACAATCTCGTTTTTTTTTGGATGCT
CCTTTGACACTATACATATTTAATTCCTTTAATATTTCATCATAGAGCAGTCCTTCAGAACCTCTTATTTTAGTTGTTCCTTTTACTTTGGCAA
AAATATAAGACTAAAGAAGCTGCAAATCTTGTTTTCCTGCCTATTTAAACAGCATCTAGAAATCTGAAGTAATTGTCTCCTCTTATTTTAAAGG
TTTTTTTTTAAAAAAAAAAAAACAGCATACTAAATATTGTGGCATGGAGTTGAAAGCTTTAGGTCTGCAGCATTTGAAATGCTCAAATCCTGTC
TTTAAAGAACAGTATTTCCTCTTTGTGACTCAGTGCTGTTATCAATATACAAGGCTGATAGAGTAATTTCAAATTCACAACATTATATGTAGAT
ATTCAAAGGAAAGAAAAAGAGCTTCATGTGACTTTGCTTAATGGGTTTACCTATAGTTGTAAATGTATACTGTAAGTGATCCATATTTGGGAAT
TATCATGTAACAATTATTTCTAATAATAATTGATATAATCTATTAGAACTTGTCAGAGGTTTCGATGTAAATAATTTACCTGCCTAGCAGACAG
AAGGGAAGGAGAGTAAGCAGGGGAAAATGTTTCTAAAATGTCCTATCATTGGAAGTAAATGATCATAAATGTTTTTGCGTTAAGTTAGGGTTTG
GTTTTAATTTTCCCTTGAATTCTAATGTTCCTACACCTTCCAAATATATTTTAAATCTTCATTAAATAAATCACATTTTAATCACACACGGAGC
TTAAAACAGGTGATTCTATGACAGATATTCAGTATTAGCTAGTGAAGGAGAACATTGGTTGAAGGTACCTAAAACCACGTGCATAAGGAACAGT
TTATTTCTTATTTGGTGATTTACTATTTCAAGTCATTTTTGTGTCAAACCTATAAAAATCTCCAGTGCTAAAAAGTGTAAGAAGAAATGAGCTA
GCAAAAAAAAAAAAGTCGGGGGAAAGTTAATTTTTAAACTTGCATTACTTTTATTTTCCTACTTTCTGTTATTTCAGAATGCATACTTGGAATG
AATCCTTCTAGAGACGTCCACGGTATGTTAAATTCCAAGTTATATTTTTAAATAAAGTACACACCTTCATAGATGTGTATATGATTTTATATCT
ATGTATCTCTATGTATAACTTGTCATATAATTTAACCAAATAATTAAGCCTGTTTCTGCTGATTCCAATATACTTAAAAGCAGTTCTCCATATT
GTAAATGTTATACCATATAAAATATTTCAATGAAAGCTTAAATGATACCCTCAGTTCTACAGCGTTTACCATAGGTGCATTCTGATGCTATTAA
ATGCCTCTATACAACTCTCTAAAAACAAAACAGAGCTTGGCTTCCCTCACAGCCCAGCTTATTACCTGTGTTATAGCAGTGGTAATGCAAGATT
GTTATGAGAGTAAAATGTTTACTAAGCATTCCTAAATTTTAAAAAGATTAATTTTAAAATGTCCTCAGCTTGCTACTTCTGAGGTACACAATGG
GTTTATGAAGTGAAAGTGAAATCATCACACATTAACCATAGCATCCCACAGAAACATTCAGATGGAGGAAGGATGTAATGGAAGAACTAAGGGC
TCTCCTCCCCCCTCTCATAGTAATTAGATTTCAAAAGTGAAGTAATCTCAAAGCCACTTCAGAAGAAAAAAATGTTTAATTAAATTCCAATGTT
CAAAACTGGATAGTGTGTATGGCAGGTGATTTGTACATACATGTTACTCTTCATCAAAATTCTTTTCCATCCCTGTGACATCAATACAAACTGC
AGCTATTTGGTTCCAAACCATACCAAGATACATTCTATTTTTTAAAATGTAAATGGTCATTTAAAATACAAATAATTCTTTTAGACGTACTCCA
TTTTTACAAATGTGATTTTGGAAATATATTGCTCGCAAAGGGTAATTTTAAGAGAATTGTTGAGATTCTAATCCCATCTTTATGCATATTTTGG
GGAGGCTTTTGAAAGAGCAAAAAGGTAAAAGATTTAGTTATCTTCTCAGACCACCTATAGTTCTTTACCTAAAGTTGTAGAAGAAAAACGTCAA
TGCACTGTTACTGTTTTCCAATGAAATTATTTTTCTTCCAACTAACCAAGCAGTACTTTTTAAATCCACTGCAGAAATGTACATTTCATTCCTG
TTGTGAGCCGTTTTCAATTCTCCTCCCTCCCTCTCCCCGCAACAAAATTTGGAAAAATCTGTCACGTAGCATCCGCCCAACGTATCGCTTCGTT
GTATTTTTTTTTTTTTTAATTTTGCATTATGACAAAAGTAAATTCTAGCATTACTCCAGGGAAACACTGGGAATGAACCTGGTAATGTTTCCTT
TCCTTTCTCCCTTAGGCAAAGGAAGGGGAATTCTGTATTGTCGCTAAAATGAGCGCTCTTGGTGGAAAGGAATTCCTCACTTCGTACACGCTGT
GGCTGTCCTTGAAAGTGCAGGCTCCGTAAAATGCCCGGCCGACTAAGGGGTTCCCTTTCCCCGGAGCCCCTGCCTCTTTCCATTCATTTTAGAT
CCTCAGTAAGGAAGGGCAGAGGTTGGTCTGTTTCTTCTTTTCAAGAGGGCACCGCGCTGCGCAGTTAACAGCCTGCGCCGCTCCGGGGAAACCC
AAATGTCCCGCGATCCCATCGAGTCAAGGAGCAAAAGCTGTGATTGCTATCAGCTTTTTGTCCGCCTTCAAACTCGGGGTTTATCCGTTGAAAC
TCCTAGCTGAGCAGCTCCCACGACGCTGCCTCTGACGGCTAAACTCGGTGCATGCAAGGCTGCGACCAACTTTCCTTACTCGATAAAGGAACTT
GACTCCCTCCTGGGAGACCGCAGCCCGAGTCCGACTCGCAGCCCCCGTCGCGGGGTTTAGGGTTTTAGGCAAGCGGCCCGAGACCCAGAGGAGC
CTCCCTGGACCTTTCCGAGGGCCAAATCACCCGAGTCTGCTATTTCCCCTGGAGTCAAAAGCCTACTTTTGGAGCCGCCACACCCTCGTGCTCC
CTCCCGCATCTCGGGGGTTCCGGGGGATGCACGTGGGGGTGGCCCCAGGAGCCCTGGGTGGGATCTCGGGGAAATGCATTGGAGACCAGGCGAA
AAGGTTTCCCTTGCCCAGAACTTGCCTCTAGAGCGGGCCGCGTCCGAACCCGCAGGCAAAGGCGCTGCGGCCCCTGCGGGCGGGCGGGCGCGCG
CGATGCACTCTTCTCAGGGACACGAGTTAGAAAGTGGACGTTTGGGCGGGGGAGGGGGCGATTTACAACTGGAATTAGGCTTAAAAAAATCATT
TTAAAAGAAAAATGTAGTAAGTTTTCTTTCCGAGACCCCAGGTCCATTAAAACAAAACTGAACTCGGGTGCTGQCAGTGCCCAGGGGGAGCGGA
GGCGGAGACGGTTGAGGCCGCGGGCCGCCCGCCTTAGCCCCCCGGAAGCCCGGGCTCCTGAAGCACAGATAGCCAAGAGCGGCCCGAGTCAAGC
CCCAGGAACAGGGGCTTCGGGAAGACACCCCGACCGGCCCGCACAGCGGGGAGGCCTGGTCCGAGAGTCCCCACCTGGCCGGCTGCGACTCTAT
ATTGGAACCTCGGAAACGCGCCTTGGCCGTTCCAGCTCAAGTGGGCTCTCGGGCGGCTTCCTGTCTTTTATCCTCGAACGCTACTTCTTCCCGG
GCCACCCGAAGGCCACGAGGCGGGCCGCACAGCCCCGCGCCGTCGGGGGCGTTCCGGGAGCTCCGGGGACTGCGCGCGGCGTCCAGGGAGGCCG
GCTGCTGCCCCCTGGCGTCCGCCAGAGCCCGCGGCGCGTCCCGACAGCTCCGCGCAGTCCGGGCCCGCCCGGGAAAGGCCTCCACGCCAGTCAA
CCCCGCCCCCCTCCTGCGTCCGGGCCCGCGGCAACGCCCGGGGACACTCCCTGCGCGGCAGCGGCCACAGCACCCTAGCCCTGGGGGAGGGAGG
AGCGGGCGTCAGGGCCCCCGTGAGGTCGGCGAGGCACCCCCGCGCCGGGGAGCTTAATTGAGGGGCTCCGCGCGGACCACAAAGCAGCCCGCGG
TGGAACGGGGCCGGGATCCGGGCACTGGGTTGCAGCCCCGGGGTGGATGAGAGGCCCCGGGAGGTGCAGGGCCCGGCAGGGAGGGCGTGGAGGG
ACTCATGGTGACGGGAGCAGCCTCCGCTGGGAGGGAGCCCCCCCGGGCGGGGTCGGAGTTGCCAGCGCACGCCGGACCCCGGGCGGGGCCCTCC
GACCTCCGCCAAAATGCCCGCAGCACCGCGGCCACCGGCGCAGGGGGAAGCGCCCCCCCGCACGCGAAGCCGGGGTGGGTTCCGCACCTGGGGC
CGCTCGAGCGCACGGGGGCGGGGAGAGGAGCGGGGGCCGCTTCCGGGGAGCCGGAGGGGAGACGCGGGGCCCGGGCAGGCTGTCCGGCTGCTGC
GGGTGCCGGCGCTACAAGGCCCGGGAGCAAGCCGCGTGGCTGCTTTCAACTTTCCTTCCAGCCGCGCGTTCTTGGGGTCTTTCCTGACTAGGAA
ACTCTTCGCTGGCCGCTGACGCACGCAGCAAGTGAGACGCGCGGAGGGATCGAGCCGCGGCCCGGCCGCAGCGGTAAAAAACACCCATTACAGA
AACCGACCCAAGGGAAGACCCTGAGAGCAAACGGCACGCGCCGACGCCGTCCGGCCTCATGCAAGGAATCAGCGCCGCTTCGCCGCAGCTGCCA
GCCGGACGGTGCAGGAGCGCGGAGTGGCTCCCCCGGGCCCCGCGGCCAAATCGCACCCCCGCCCTGGAACCGGAGGCCTGGCCCTGGGCGGCCG
CGCAGGATCTGGGGCCGTCCGCCGGCTGCAGCCCGGTTCGGCACCCTCGTGGCGGCCACTGCGCCTGCCTGCAGCGGCTCGCGGGGCCGGGCCT
CTCCGGGTGCGGCGCGCCGGCCGGGTCCTCCTCGGGCCGGCGGGGCCTGCTTTCTTTTTCCAGGTCTCCCGACCGTGGCCACTCGCGGAGCCGC
ACAAAGCCTGCGTGTGGCTGCGGAAACGGGTCCGCACGCGGCGGGGCCCCGGGCCAGCATGCAGGCTTGGATGCGGGGCGCGGGGGTGGCCTCC
GACTCCCCGCCCGGGCTGCGTACAGTAGCGCGCGGCGCGGCGGCGGGGGCGCGCGGGCCAATTCCAGGAGCCGGCGGCGGCGAGCCGCGCCCTG
GCTGATACCGGAAAGGCCTGTGATTGGCCGCCGGCCGCCCCTATGCAAACGAGCTGAGGGAATGGAATTCCTCCGGCCGGGCTTACAGTAAAAG
CACGTTCATTTCCAGGCACTCTCATTCATAGAGCCAGCGGGCGCGGGCGGGACGGGCGCCCCGCGGCCGGACCCAGCCAGGGCACCACGCTGCC
CGGCCCTGCGCCGCCAGGCACTTCTTTCCCGCGCTCCTAGGGACCCCAGAAGGAAGTCAACCTCTGCTCCTTCTCCTTGGCCTGCCTTGGACCT
TCCTTTTTTTGTTGTTTTTTTTTGTTTTTCCCCTTTCTTCCTTTTGAATTAACTGGCTTCTTGGCTGGATGTTTTCAACTTCTTTCCTGGCTGC
GAACTTTTCCCCAATTGTTTTCCTTTTACAACAGGGGGAGAAAGTGCTCTGTGGTCCGAGGCGAGCCGTGAAGTTGCGTGTGCGTGGCAGTGTG
CGTCGCAGGATGTGCGTGCGTGTGTAACCCGAGCCGCCCGATCTGTTTCGATCTGCGCCGCGGAGCCCTCCCTCAAGGCCCGCTCCACCTGCTG
CGGTTACGCGGCGCTCCTCGGTGTTCGTGCCTCGGAGCAGCTAACCGGCGGGTGCTGGGCCACGGTGGAGGAGTATCCTCTCGCTGCTGCCCCA
CTCAGCGCTGAGTCACCCAGCTGATGTAGACAGTGGCTGCCTTCCGAAGAGTGCGTGTTTGCATGTGTGTCACTCTGCGGCTGCTCAACTCCCA
ACAAACCAGAGGACCAGCCACAAACTTAACCAACATCCCCAAACCCGAGTTCACAGATGTGGGAGAGCTGTAGAACCCTGAGTGTCATCGACTG
GGCCTTCTTATGATTGTTGTTTTAAGATTAGCTGAAGATCTCTGAAACGCTGAATTTTCTGCACTGAGCGTTTTGACAGAATTCATTGAGAGAA
CAGAGAACATGACAAGTACTTCTAGCTCAGCACTGCTCCAACTACTGAAGCTGATTTTCAAGCCTACTTAAAAAAATCTGCAGCGTACATTAAT
GGATTTCTGTTGTGTTTAAATTCTCCACAGATTGTATTGTAAATATT1TATGAAGTAGAGCATATGTATATATTTATATATACGTGCACATACA
TTAGTAGCACTACCTTTGGAAGTCTCAGCTCTTGCTTTTCGGGACTGAAGCCAGTTTTGCATGATAAAAGTGGCCTTGTTACGGGAGATAATTG
TGTTCTGTTGGGACTTTAGACAAAACTCACCTGCAAAAAACTGACAGGCATTAACTACTGGAACTTCCAAATAATGTGTTTGCTGATCGTTTTA
CTCTTCGCATAAATATTTTAGGAAGTGTATGAGAATTTTGCCTTCAGGAACTTTTCTAACAGCCAAAGACAGAACTTAACCTCTGCAAGCAAGA
TTCGTGGAAGATAGTCTCCACTTTTTAATGCACTAAGCAATCGGTTGCTAGGAGCCCATCCTGGGTCAGAGGCCGATCCGCAGAACCAGAACGT
TTTCCCCTCCTGGACTGTTAGTAACTTAGTCTCCCTCCTCCCCTAACCACCCCCGCCCCCCCCCACCCCCCGCAGTAATAAAGGCCCCTGAACG
TGTATGTTGGTCTCCCGGGAGCTGCTTGCTGAAGATCCGCGCCCCTGTCGCCGTCTGGTAGGAGCTGTTTGCAGGGTCCTAACTCAATCGGCTT
GTTGTGATGCCTATCCCCGTAGATGCCAGCACGAGCCGCCGCTTCACCCCCCCTTCCACCGCGCTGAGCCCAGGCAAGATGAGCGAGGCGTTGC
CGCTGGGCGCCCCGGACGCCGGCGCTGCCCTGGCCGGCAAGCTGAGGAGCGGCGACCGCAGCATGGTCGAGGTGCTGGCCGACCACCCGGGCGA
GCTGGTGCGCACCGACAGCCCCAACTTCCTCTGCTCCGTGCTGCCTACGCACTGGCGCTGCAACAAGACCCTGCCCATCGCTTTCAAGGTACTG
GCCCGGAGAGGGTGGTGGGAGGACAGGCGGAGGCCGGCGAGGCCGGGGGAGATCCGCGAGGGCAGCGGCCAGGCGGGCGGGGCCGGATTCTGGA
AAGGGGCCCCCGCAGCCCCGGGTCGGTCTTCCTAGCTTGCGATGTGGGGGATGCTGTGTTGCCGTGGCCACCCTCGGGAGGCACTCGTCTCAGC
TTTCTGGGGCGGCCTCCCAGGAGGGCTGAAAACATCAGGCTGTCCCCAAAGATATCCACTGGGAGCCCCTCGGGACCTCCCTGGCAGGCGCCTT
AGCTTTGATACAAAGTAGGAAAAGTTATAACCCAGCCCGGCAGAGACAGCCCGTCTGCTAACTTACTGCTGCCCCTACCGCTGGATTAACACCC
AAACCATTTTGTCTTCCTCAGATGAAAGGATCAGGAAATAAAATAGTAAGACTTTTCTGCCTTCAAGTCAGTGAAAAGAAACTAACTAGGGAAA
CTGTCAGGGATTTTTTTTCCCCTTCTCTAACCGCCCTATAGGAATTAGCATGTCCAAAGTCCACAGCGGGCGTAGTTAAGTTTGCAAAAGTTTT
CTGAAGAGTTCTCTGAGCTTTTTAGCCTGCTCCTGCCAGAAGCCGCAAGTATCGAATCCTGTTTCTTTTCCCGGAACCGTTCTGAATGCTATCA
TCAGCTCACCTAGATTAAGCCAGGGGTAGCCAAAGGTGACGAGGCTGTTTTCGGAGAGCCAGGGGTCCCAGCGGCCCGCTTTCCTGCTAGAGGA
GGGGGCGGCGCGTAGCCGGAGACTTCAGCGCGGCGGCGCCAAAGATCTCCCTTCACTTTTCACCCCTGATCATCCCCAAAGCTTTGAGCTGCCT
TTAAAAACCTTGACCTTCAACAGGGGTATGAAAACAACCCCAGGCGTTTCTAAACAATCACCCTACTACACTAGCAGCAAGCAGTAGGAATTTA
ATTTCTGCAGTGGAGATTCTGTTTTGTCTTTGGTGGGTTTGAAAAGATGCGACTAAGTAAAATCAGTGCGGGTCATTTCCTGTTCGTTTAAAGG
GCGAATCAAGGCAGGTGGTTCCAAGTCTAAAGATAAGCTACTAAAAAAAAAGTTTAAATAATTGTCCCCTTTAAAGGGAGATTAATTTAATATA
AGCTTTTTATAGTTCCAAGCCTCAGCTGGCTCTTGATCTTATTACAACGACGAATATTCACTTTTTAAAAAAGTATTGTTGCGGTTATTTTTAA
GCTTTATACAGTTTCCACACTTAGATTGTGATTTCTAAAACAAAACGAAATTTGCAGTTCCCTGTGTCTATAGATTGGCCTCTTTTTCCACTCA
CTTTTCTAAAAGCAGTTTTTTTTGTTTGTTTTGTrTTTTAGTGGGGGTGGAAATGAGGAGAAGCAACGAAGGAACGTCCTGGCTTGCTTGACTC
TTTTTTCCCTCCCTCCGTTTACTTAGATTTGGGTTTGGGAGACCATTTTACTTTTAAATCATTCTAACCCTTTGCCTAAATGGACTCAGCGATG
ACTCTTTAGGGCACCCGCATTGAGAGGGGACCCAACAGGGTTGGGCCATGGGGTCACTATCAGATAAAGAGCAGAGGCAAGACGGGCTGATAAC
CACCAAAGGCCATTAAAACCTGGCTGCCCCAATGCACTAGGGCTCTAAGAGATTTGTGGCTTCCTTTTGGGGGGGAAATCAGCGTCTCTTCTGA
ACTCCCTCCTCTGCCAGCGAACCCACACTCAAATATATTAAAAGACTAAATCATTAATAAATAATCTTCAGCAATTGGAGCAATTCAGAGTCTC
AAGCCATTACATGAAAGTATTTATTTTCTTCTAAAAAATGAAAAATTATACATATTGCTTTTATGAGCTGTCAGAATTCTTTCTAGCAGAAACC
ATAATAACCATAGAGTTTCCTTTTTAAAATGACTTTTGGCAATTCACTTAAAAACAATTAATTTGTTTTATTCAGTGATAATCTATACGCTACT
AAAAATCCAAATACAGAGCTCTTGAAAAAAGTTCAACCACATCCCCTGAGAACATTTACGGGAAAGGAAAGCATAGCAGAGAAATCACACCATT
TGATTATTGTCTGTCACCACCCCAATCCTCATAAAAAGACGCTTCTTCCTGCAGCAGACCTGCTCACTGCATAACTAGAAGAGTAGACAAAATG
TTCAGTGAGACAGTAAACGTCAAGGGATAAAATGTTAGGCATAAAAGCAAGCAAAACATCAAAACCTGCCTACATCCCGGQCTCATTTAGTGTG
TGTCCCTGGCTTCACTCTATCTGCCCACCCAACCACCTCACAAGCAGCTTCAACACAAATTATTACTTTAAAATTTGGATCTTAAATAAGCCGC
AGGCAATCTCCCTAGGAAGTTAACCAGTAAATAATTATTATATTCCCTATGGTAAAGAGAAGCAAATGTGAAACAATGATAAGGCCTCATGATA
TTGATGAAAAGATCACTCAAGGCCTTCTTAGGAATCTCCTGAAGTTCTGTACGTAAATCCTCTACGTCAATCTTCTGAACTAAAGAGAGATTTA
AAATTATGAATTTTCTTTAAAATAGATTACAATAACCCTACTTGCTTATTTTGAGCCCCAACCTGTTCTCCCCCTACTCCTTCCCTCCCATGGA
ACACCTTTATTTTTTACAGTATGAGAGATACATGTTTTTTACTACTAAGAAAACCTTAAGTTTGCTGTGTGTTTTGCTGTACTTACAGTATTAG
CCTAAAAAGCTGTTTTGATGACTTAGGATGTATTTGGTAATACCATGGTGTTATAGCTTGTTCAGGCCCTGGTACACCAGAGTCCAAAGACGTA
AACGTGCCTTCAGCAGGGAAGTTCGGAGGTCTTGGATTCTGATGGTAATATTCTTTTCATAAAAAGGAAATAAGTAATATTTTTGTCGGCAAGA
TCACAGTCCCTAGCTATGCCTTAAATATCATCTCAGCTGAATTCTGTGTTCCACTCCACAGCCTTCTTTGCAACACTGGAAGGTTAACGGATTT
GAAGTAAATGAATTGTAACTGTAATTTCAACCTAATTAGAATTCGTTTAAAATGCCAGTTTGGACCGTGAACAGGCTAGCCAAGAACACTATTG
GGAGGTCCATGTAATATAGAAGAGAGTTGATATTTGGCCCAACTTCCTGATACTTTAAAAACAGAGAAGTAAAACAAAATATGAGATATCTTTG
GCCAGCAAGTCCCTTCTTGCCTGTTTGGTCTGTGTTAAAATGTAAATGTTTGTGTTTCCTTACCTATTCTTTATAGCTTTTCATTCCAGTTGTT
TCTTTTTTCTGTTTCAAGTTAGATATTTTTACATTCTCTCCATATTGCCCATCAGTGACAAAACCTGTATCAAATTAGCACGCATGTTAGTTTT
GAATTAAAAAGAAACTTTTTAAACCTGGCTTAAGTATCCTTGTTAACAATTGGACGATATACAGTATTAATGAATTGCCCTGCATTTACTAGTT
CCCCTTTACCCACCCGCCTCCACCAAAAAAAAACCCTCAGAGATCAATACAAGGCCAAGTGAATCATATGTAGGTGCCCAAATATGGTTGATTC
TGCTGCATTTAAAAAAAAAGAGGACTGGGAGTTAAACTTCTAATTTTTTTCTTCTTAAGTTCCAACATCAACAGCCTTTTCTTATTTAGCCTCC
ATGATAATTTTATTCTATGTTTGTTTCGACTTGGCGAAGGTCTCCTGAATTTGCCAAATTACCAATGAGGATAGTATTGGAAAGATGAGAGAGG
CACAGGCTTGGAGCACTGTGAGGGGAACAGCAGTAGGAGGCAGATAATTCAGAATACATTCCTGTAGTTATAAAAGAACATTTATTTATTTTCA
CCTTTTTACAGATCCAGACCCAACAATGAGTTATTCAGTTTGAAACCCACGTAGTGGACGCAGTCTCTGTATACCTGCTGAATGTTGAGTTACA
ATAGACAAGCCTGGGTTACCACAGGGAGAGGAGGATGAGGAAGTAAAAATATGTGTTCAAAGAGCCCATTTCAACACTGTGAGCTAACTTTACA
ACGGGAAGTGAAAATCATGAAAAAAAGGGGAAAATTTGTTGTGAGAGACTAAAAAGAGCAAAATAAAGCAAGCCCATTTTTCGGGAATAGGAAA
AAAAAAAAAACAGTAGCAATAACCAAATAACTGATCAGGGAGATTTATTTCCGTGATATCTTTTTAAAACAAAAAGGGAATTGTCGTATCACTG
CTCTTAATTAAATGGCAACAAACACACTTATAATGGGAACTAAATAGCTAATTAGAGGGTGCTTGGGGAATAAATAGCAGAGTTTTAAAATCTC
CTATATGGAAAACATGTCCAACAATTGGAATGAAAGGATTAAAAAGAGAGAAAAGGCCCAGACCTTGTTTGGCAGTGCAAGTTGGATAAAATAG
AGGAAAATCCCTCCCTGCTTCTATGAGACTAGATTATCCTGGTCTAGTCTTGGGGTTGCAAGTATGTTCATCTCTCATAGTGAGTTAGTTCGTG
TTCTGTGCCACAAGGACTGAAGCAGTTCTAATGAATAAAAACAATACATAGTCATCTGGAZGCCACAGGGAAAGGGTGATGGCTAAGCAGATCA
TGAAAATGGGAATCTTGACAAACATGACTTCGATATCATACAAGTGAGTCTATCGCAGCCTTTTTTACATATAGAAAAAGAGCCCTGAAAATAT
ATACTACAAAAAATTATGTAGCAGTTATTAACGTAAAAATTGGGAAGTACGAGGCAGAGGAAGGAGTGATAACACTACATTAAGGAATCCTTTA
CTGTTACCAATTCAAAAACAGCAACCAAAAATGATGATGTAATTGGAATGAGTAATATAAATGTCACTACTGACTTTACCGTAAAGAATGTCAC
AAATGATCACAATAAATACAGCTTGCCGCATTTCGATTTGAAGTTGTATGACTGCTTTGAAAAATAGAAACAAAGGAAATACAGCCAATAGTAA
ATGTCTAGACCAAGAAAATAGTTTCTAGAAGGCCTAAATGATAGCCAAGAAATTGATGCTATTGTACAAACTATCTTTAGGTCATGCTTTTCAG
AGTATTTTACTTTGGAATTCATTTTCTTAATTACCTGAGTATAAAAACATTTATCTACCAGTTTTCTCCAATAGCCCTGATAAGACTTCATACT
TACTCAGTTACATTAAATAAATTCCTGGTCAAGATCAGCTGGGCTAGCAGAAAACCTCAGGCATCTGTGAGGACATGAGTTTACACACGCTGAG
ACTCACAGATACAAAAATGCAACCCAATTCCACCCCTGAATTGACGGGAGTGCATAGAAGTGAATGTCCCGTCTTTCTGAGGTCTGTTGATTTT
GTAATTAGTAAACGAAGGGTGCATTTCTGATTTTTTTTTCTTGTGTGCTAGAATTCATTGCTAGTAAAACTCAAGATAATAGCGATGAGTAGGA
GGTATCAAAGATGAACTGTAGAGGGACAGTTTAAGTTACTTAAGAATCGTCAGCAAGATGAAATCTACTTTTAGCAGAAATTGGGTTTTTTTGT
GTTTTTTTGTTTTGTTTTATTTTCTAAAAGTAAAGTCTGCACCTTGTTCAGCCTGTTAGTGGAGGTCTGAGCAAGTAAAAGATGGGTTGGATTA
TAAACTTACAAACACAGGATGTTCTGTTTCTCAAACGGGAGAAATTAAGAAGACATGCTTGTATTCACGAGACGGCATAGCTACTCAAAATCCT
TGATATCTTGCTATGGTTAGTCTTGTTCCAACTGTGCTATGTGACCTACTATGGCTTTATGAGGTAAATTTAGTATATGTGTCACTATTTGAAA
ATTTACATATAGTTATACATAATCTATTAGGTTGGTGCAAATCTAACTGCAGTTTTTCGAGTTAAAAATTGCAAACACTGCAATTACTTTTGCA
CCAACCTAAAATATAATAAAAAACTGTCTTCTCTTTATAACTTTTCTGTTGTTTTGCTTAAGTTATCATTGCTATTCCTCTGCAACCTAAAAAG
AAATCATTGAATATACATTTAATTTTAGAATAATCACTACACAAATGCCCTAAAAGTGTATGTATAACATCCCTGATGTCTGCATTTGTCCTTT
GACTGGTGTTTAGGTGGTGGCCCTACGGGATGTTCCAGATGGCACTCTGGTCACTGTCATCGCTGGCAATGATGAAAACTACTCGGCTGAGCTG
AGAAATGCTACCGCAGCCATGAAGAACCAGGTTCCAAGATTTAATGACCTCAGGTTTGTCGCTCGAACTGGAAGAGGTACCTTATCTGTCAAAA
CTATGCTTGAAACACGTTTCATGGCAACAAACCCACATTTCAAAATTCTAGTGATTTCTGTGATGGAATCCCTAGAAACTCGGTCTGTCTTAAC
ATTCTTATTGCTATGTCAAGCTCTTAAAAATATTTATTTGAAGTTTAATATTTTTAGCAGAATCTCTATATTATTGCTTTATGTTACTGAGTGG
ATTTAAGACTGATTTGCAACATGCAACGTAGGAATTGTGAATTTCAAAGAAACGCCTGAGATTTTTACTAAAATCCAAAAAATATAAAACTGAC
TTTAATGAAACTAGGATTATACAATTACAATGAGGATTCTCTCTTGGATAACAATACAAAAGCTATTTAATGGAAACTGTTGACGTATGATCTG
TGGGCTATCTGTGAAAAGTGACAGAGTTTCCAATGACCACTTCTAATAATTTTAGGGATTTCTAGTGTATGATGTTGAAACATGGTTTCAAGCA
AGCTTACCTTTTTAGAAAAAGACATGAAGAAATAATGTTAGTACCATTGATGGCAGTATGATCTTCCTCTTGTATTTCTTTTCAATGCCTTATT
TAGGAGGAGGGGTTGGGCCTGGTTAGCATTTTAGATACTAATAATATTAGAGATTGGATTATCCAAGAAATTGTTGCATTACTTCAGCTCCATG
TGTGATTTTATTTTTCCTTGCCTTTAACTCATTATCAAAGCATTAGAGCAGAAATAGGAATCTTGGCTTATTGTAATTTTTTCTGGTTTCCAAT
TATTTTCAAGTATTATGTTTTCTTTACATTGTAAGAACCAAATCATATTTTATAAATAGCAACAAGTAGTAAAAGTTTAGTTGAGGGAGGTAAG
ATAGTTTTTTAAACTCATTTTTTGTAATATAGTACCGATTAAGCTTACAAATAAGTTATTTCAAGTATAATAAAAAGACTTATCGTAAATACCT
AAATAACAGTAATAGTTCTCGTATTGAAAAAAATGGAAGCACCAATCAAACAACATTTAAAATAAGAGAAATTATTACTCTTTAAAAAGGCATT
TAAAAACAAGAAAATCTCAGTTCTCAGAGAATTTGATTAGTCAGAAAGCAGAAAGGGGTAAATGTGAGTTGATTATTTTGCTGATTATTTTTGC
TATCCTAGCTATCTGGACCATTTTCATAATAGAATCATACCTGGAGAAAATAAAAAATTTATATAATGTCATTGAAAATATATATTCATAGTAC
TATATTAATAATAGGGTAATGCAAAGAAAATTATATCCACTTTTTGAAAGCATGAGTGATGCTATAAGATATAAAAGCATAAGTCATTAAATGG
GTTCACTTGTTGTATTAAAAGTCCCCACCTTTCAGTAAATGGAATTTGCATTATGATTCAGAGATGTGTCTCTAGAAAACATCTTAAAGGCTTC
GGATCAGTAGCTGGCAGAGTGGGTGAGAAGCCAATAAAAAACAAACGCCCCACAGCCTGGTCCATGCCCATCCGCCTAGCTAACTCTCTTAATT
CAGTTCTTGTGGATTTCTGCTGTTAGGAATCAATAGCTAAGTATCATTCAATTGCTTTTCTAGAGAAAGTGTTATCATCATACTATGCATCTGT
GTGGGTCTTCAAGTTAGGTTTTGGTCATAAAAGTTTTAAAAACTATTTAGTCATCCGTGCCAGTGACATTTTCCCACAAGAAGTTCAGTAGTCC
CTTTCCGGCAATGCACAGTAAACCAGTGATAATAAAAAAATGAGATTTTGAAAGCAGTGTGCTTTCTTTTCTGGCATTGATGGCTGATCTTGCA
TCCCTCATTGACCATTTCTGTTTTGAGTCGCTGACTAATAGGTGCTTGTGTTTACTTGGGTAACAGTGAACTGTTTCCCCAACGTCTGGAGTGT
TTGCATTTGGCTGCAGGATTCGTGCCACTATGCCCTTGAGTTTGCATAAACCCCAAAAAGTAAAATACCTCCAACCAAGGCTCGGCTGTCTTCC
CATACTCTGGAATGAGAAAATTAATTGAAATGCCATAAACAGACAATATTTTGAACAGACTCTCCTTCCCACCAGTGATAATAAGCAATAAAAA
ATACGGAAACCCTAATGCAACATTTGACCCAATTGTAATTACTGTGGATATGAATATTCTTCGTCTTGAGATGTATATATATATGTGTGTGTGT
ATATATATATATATATTTTTTTTTTTTTTTTTAGTCAGCCCTACAAAAGTAAATGAGCTGTCTGAAGGCCCGATTTGACTCTGTCATAATTTCT
TTCCATAGTCATAAAATATAACTTGATTTTTCTCTTTCAAGTGTAAGTCTCAACACCAAAGCCTGCCAGATTTTTTTCTTTCTTTTTCCTTCTT
CTTCTTTTTTTTTTTTTTTGCAATTTGCTTCCCATTACGAAGAGAGCAGATCATTCTGACTAATTTTAACAATTAGTCTTAATCTTCCTCACTG
AATCTTACCTTTAACAGTTTCATTACGTTTTATTAAGTGATCTTAAGACAACTGTCTCATTTTCTGTTTTTGAAAATGTGCTTGAAAATATACC
CTTGGCATGAACACACCACGCCATGGCTCAGAAGGCCCTGGCTCTTGGCTCATCCAAACTTTCAGTGCCAATTTTGAATTCTCTCAAAAGCCAC
CAGTCTGATGGATTCTCCAAGGGGCCTAGTAAGTGGTGCAAAATGAAAGGTAGAGAAAGAAGTTCTCTTTCTGTGACCAGCTGCCAGTCCATGG
GGATTGTAAACCTCATAGAAACTGCCAGAGGTTGAACATGGAAGGGTTTTTCTCTGTTCTACAAAACGAATCTATCTGGGCGCTGGCTGCCATC
CAGGATCACAGCTCCTAACCTCGGGTTCATTCCGTAACTCCGAAAACCTGCCTGTCCGGCTTTGATGGCCTCGCTGGCTACACTGGCCCTGTAC
AAGCCTGAACCTTCTCTCTTTTCTTCCATTCCCTCCTTTTTCTCCTTGGCAGTTTAAATCTTGGTTCAGAGTGTATCTCACCCTTGGAATGTTT
CTCTCTTTGATTCAGTATCTCCATGTAAATCGACCGCTCCTTTACAAAGGAGGAAGAGATAGCAGCTCCCAGGAGATGAGGGTTAGGGAGCTCT
GGGGACCGGGCTGAACTCCAGAGGTCATCTGAGCACAGAGGTTATTCTTCAGAATAATTCACATTTTAGCCAAATGGACTGGGACTCCATGACA
CCCCCAAAAAATTGAAGTTCAGGGTCAAAAGGAATTCTCTTATGTGAAACGAATTCACTGAGCAGCTTATATCAGTTTACATCTGCAGTTGAGG
CAAGTGCTACATCACCTGCCACCTTTAGAATGACAAGAAAGTGTATCATGGCTTCAGCTGAGAGAAATTTAAGAAATTTTAGCAATTGGTATCC
GGGCACAGTGGCTCACACCTGTAATCCCAGCACTTTGGGAGGCCGAGGTCGGTGGATCACCTGAGGTCACGACTTCAAGACCAGCCTGGCCAAC
ATGGTGACACCGCCATCTCTACTAAAAATACAAAAAATTAGCCAGGTGTGGTGGCGGGCGCCTGTAATCCCAGCTACTCAGGAGGATGAGGCAG
GAGAATCGCTTCAACCCAGGAGGTAGAGGTTGCAGTGAGCCGAGGTTGCGCCACTGCACTCCAGTCTGCGCAACAAGAGCAAAACTCCATCTAA
AAAAAGAAAAAAGAAAAAGAAATTTTAGCAATGAAATCTGTGACTGCTTCATCCCAGTTGGAGTATTTAATCAGTCAGAGAATAACTGATCAAT
CAGGAAATACAGCTTTAGTAATTTCATTTCAGAACCAAAGAAGAAATCTCTCCAGCTGCTTTATCAGCTGCATTGTTGCTGCAGGAGGAAAGAT
GGTCTACTGCACATTTCTTTCTAGTTTTTTCTTAGAATATGATTTTTAATCTTTGGGGAACATCATAGATACTTTTGAAAATCCTTTGGAAGGC
TTTTTCCAAGGACAAAAAAAAAATGCACGTGTTCACTTACACACAGAATTACGTATATCATTTCAGTTTCTAGGCTACCAGAACTCATTTATGG
ACCTTCTGCGGATCCACAGACCCTACGCCAAAAATTTGTGAATTTGCAACTTTGCATTGATCATGCAGGGCCAGCTTAGCCCTATGCCGCATAT
AGTAGACACCTGAAAATGAATAAATACATGTACAAATGAGCTAGACATTTTCTTGAACCTGTGCTGTTTTGGTAACACCTGTGTTTTTGTTATC
GCCGGGGTCTGGTTTGTCTAATTAAGAAAGTAGATCTCCTTGAAACCACTGGCCAGGCCTTTGGATGGCCATCAGCTATGCCCACATATCATCA
CGCGTAAAGGGGAGGGGGCCTATTCTGTCATTGAAAGGACAGTCGCTTCCCTCGTACTGTGTCCACACCCAGCCCTGTAAGGCCAAGTGTTCTG
TGCCCTGTCAGGATGGCGTTGAGTTTCTTGGCGGAATTTGTGTTTACATTGACCTCCCTGGCAGCTTTAATCTTGCCCACTTTCAAAATAGTAT
CCTTTCCCAACACATTThTACAATGAAATTCCTTCGGTATGTTCTTGCTGTGAATATCTTCCCAGACATAATATTTATCCTGTCACAATCTGGG
TTATTGCATATTTGTGTAATGTTAGTTGCTTAAGCCATTTTCATTCTTAAATTATTCTACATTATTTGGGCTAAAGTGTGGGAGGTTGCCCTTC
AGGTGAGTGGAAGACTGTGGTCAAGAGGAAGGGAAGGTACATTTAGGGAAGGATAAACGGTACTGATATTCTAAACAAAGAGACATGGCTTGGC
TTTATGTAGCGGTTGTTACCAAGAGATGATCTGAATGACTCGCAGTATTATCGTAGGATCCCAGTCCTTCTCTATTCTATTAACACATTCATAT
AATAGAAGAAGGAAAAATTCATCTGACCACCCCAGAGTTGTTTCACGGGTAATGTTCATGGTATAAACTGGCAGTCAGATCTCCTGGAGAGAAT
ATAAGGAAAAGACATGAGTTATATTAAAATCAACTCTCCCATTTTCCCACTTTCTTCTGTTACATACTGATCTACACTTTAAAATAATATATAT
ACTGAAATCTGTATTCAGATCTCATTGCAAGCAAATATAGATGAATAAAGACCCAGTCTCTGATATACCAAGCCTCAACCAGCCCTAACAAATG
CTGCTTTTCGACGGGCAGGTCAAAAGCTTGCCTCCATGATCTGATGTAGAATTCATCTCTAGAGACCAAGTTTCTCATTTCGGTTTCACATTTG
CCAATAAAAGGATAGCAACACTTTCCTAAAGCAAGTTGCTCCCTGCCCTGCTTAAAGCCTGGGGTGGCCCAGTCATGGCAAGGACCAGCAGGAA
AAGCTTTCTTGAAAACTGCTTGTGCTTGCAACAGGCAGTTTCGCAGTTGATCCCTTCATAGTTTTCTTGCTGATTTTGTGAAGTGTCAGGCATT
GCTGATGGCGTCACACAGCTTTTTTAAATTGAGGTTATTTGTATGAACAATATTTTATAGTTTGATGGGCAGAAGCTGTACTTCTCTCCACCCA
CCCTCAACCCCTTCCTTGCCAGCCGCCTTGTGTTTGGTGGAGGCATTTCTTTTTGGTCGGGACAGAAGACTATCAATCAGAAAGTGCCTGCAAC
AGAAATAATGGTTGAGTGATTTTTAGAGCTTCCTGTTTCACCAACAAGCATTATAGACATAAACAGTGATCCAGCCAACGGCTGCAACCCTGCT
CTGGTTCTAATAAATCAGTTTTGTATTTTCTTTTTTGTGCTGGTTGGCAAGGGGCAAGAGAGGGTGGAGAAGGTTGGCTAAAGAAGAACATATT
CTGTGTTTCTGGTTTTTTTGTTGTTTTTTTTTTAACGAAAAATTACTTGTTGACAACGAGAGATGGTGTACTGTCTTTTTTTTTTTTTTTTTTT
TTTTTTGAGACAGAGTTTCCCTCTTCTTGCCTAGGCTGGAGTGCAGTCGTGTGATCTCAGCTCACTCCAACCTCCACCTCCCAGGTTCAAGCGA
TTCTCCTACCTCGGCCTCCCAAGTAGCTGCGGATTACAGGCATCCACCACCACGCCTGGCTAATTTTGTATTTTTAGTAGAGACGAGGTTTCAC
CATGTTGACTGGGCTCGTCTTGAACTCCTGATCTCGGGTGGTCCACCTGCCTTGGCCTCCCAAAGTGCTCGGATTACAGTCGTGAGCCACCGTA
CCTGGCCGGTGTACTGTCTTAAGGCTGTTTTCTTTCTGTAGATGATTATGACACAAGTTCAGATCAGTAAAGAGATGTTTATCAATAATCATAT
ACTGTCAATATGGGGAGAGTGTATGCTTTTGAAGGCTTCATTGATTCTTAGCCAGTGGACTGTGTGTGCGTGTGTGTGTGCGTGTGTGCAAGGG
GGAGAGAGAGAGAGAGAGAGAGAATGAGAATATCCATACTTCCATTGAGAGACTTCTTGCGCCAAATTATCTTTGAGGACTAAATTCATGCTAA
TAACATCTTCTCATCTAAAGCCCAAATAAAGCAATTAATGGAAATTAATAAGTGAGCAGTGTGGATTTAATGAGCTGCCTATGAATACTATGAC
ATGCGCTCGTTTTGTCGGCTCCCTGGCGGCCCATGGGGACAGATCGCAGCACTTCAACGTCTGTATTTATAAACATGCATTCCTGCACTTCAGC
CAGCGCGCATACTCCATCATTGGTTCTGCTGTGAGTCCTACATTCAACGGAGATGTATTTTGATCTCTGTTGAAGACAGAGCAATCGACGGCAT
TCAGTTTGCACTGCACTGATTTGGGATTCCAGTACATTGCCCAGTCCCTGTTAGTCTATATTGCGCAGCATGTCCCATTCAGGAGCTCCATTTC
ACATCACCGAACGTTCTCCAATGAAGGTGTCAGGCAGTTGAGGTCCATAGAGGAATCCCAGCAACTTCCTTTGAGAGGCTCACGGCCATTCTGC
TGCTAGACTGAAAATGCGTTTCCTCTGATTTCCTTAGTGAGGAAGCATTCATTTAGGAGCCATTTAAACTGTGTTTTTCTCAAAGAAGATGTAA
GTACATTTCCTGTTTATTTCTCAACTTAGATTAATTTGGGCTTTTACCTTGAGATTTAAGACCATCATTAAGAATGTATGGGCAGGGCCATCTG
CAGAACTACCCCACCTCCCCCATCACCTATCCCAAGGACGTGCCTGTCCAGTTAATACGAGGGCTGGCCACCACTCTCGGGTGGTGTATGGCCC
TAATTGACTCACCCTATGCAAACCAAGGTAAATCACAGGAATATGGGCAGATTGAGCATAATTGCTCATCTGTGTGCTCATCAGATCTCTGGAG
GTGCAAACACTCCAGTTTGCTCAAGGCATTGGGTTGGGGTCTTTACAGATTTTTTTCCTTCATTCTATTTGAAACTATTGTATAGAGGATAGTC
AGTATTAATCATCTATCTTTCTATCTATCTATAATTAGTAAGGAGCTAGTCTAACTCACATCATAATAAGGCTTTGTCTGCCTCTTTAAATGGT
GTGTCTCTAGTACCTAGGACAAATGTCCTGCCTTCATTATTCTATCTGAAACCACTGCGCAGTTTATTATCAGTATCTTGTCTCCCTGCTTATC
AATTTTGATCATAATCTTGCTCAGTGTTTCCCAGCTGGGGTGATTTGACTCCCAGAAGACATTTCGCAATATCTGCAGACATTTTTGGTGGTCG
TGTATTGGGTTGGGGAGTGCCGGTGGCATCTAGTAAGTAGAGGCCAGGCATCTACTAAACGTCCTACCATGAACCTGACAGCCTCCCCAACAAA
CACCCAAATGTCAGTTGTGCTGAGATTCACAAACTCATCTAGCTCCAGTCATGCGTGATCTCGCCCTCATCATGCATAGGTTGTAAAATGTTTC
CCTTGCCCCTTTTATTCTGACATTCTGCCTCCCTACAAACTTATCAAAATGTATGAAAAACTTATTTTAAATTTTATGAAAAACTTATTTTTAA
AACATTGCACAGAATAAAAAATAGAGAACATCACTGAATATTAAAAGTTTTTTTTAAAGGGAAAACAGCAGTGTTCTGTATGCATAGAATTGCA
CCATCCCCCTGGCGCCACTGACCCTGCCTGCTATCCTGGGTCAGGGATGTGCAGTGGCTGGAGGAGTGGCTGCAGGTCGGGGTGGTACACACAG
CAGGGCAGCTTCTCCGAGAAGGCTCCCCACTGTAGCCACCCCCTCATGAATGTCACACAGGTGAGGTCCACGAGCACTGCAGAAACTAGGAAGC
CCTCTGCCAGAGGCTCACTGCTTTTCCCTGAATACTTCAGCAGAGAGTCCATGGAGCCACTCATCTAGGAATGGCAATTGATGTCAGTGGACAG
TGTCCACTCATATTAAAAAAGTGATAAAGAGCCCGGCATGCTGGCTCACACCTGTAATCCCAGCACTTTAGGAGGCCAAGCCAGGAGGATCATC
TGAGGTCAGGAGTTTGAGACCAGCTTGGCCAACGTGGTCAAACCCCATCTCTACTAAAAATACAAAAATTAGCCAGGAGTGGTGGCACATACCT
GTAATCCCAGCTGCTCTGAAGGCTGAGGCAGGAAAATTGCTTGAACCAGGGAGGCGGAGCTTGCAGTGAGCCGAGATTATCCCAACTGCGCTCG
GCCTGGGTGACAGAGCAAGACTCCGTCTAAAAAAAAAAAAAAAAGTGATAAAAGCAAGGTCTTGGTGTGTGTCACTGACAGACAACTCCCAGAC
ACCAAATCGCTCAGTCTGCATACCCTAATAAAATGAAGGCACTACCTGGCAGGAGGGGAGACAGGACCAAGGTTTGTGCCTTTGCCGAGGCCCA
CAGGATGGGCTTGCAGATGCCACCCGCAGACAGATGGCTGGGTTCCTCTGGAGCTTTATCCCACCTGCCCCAGCATGTCAGTTTTGCCATCACC
GGTTTTTACACTGAGATTCAGGGCACCATGATTTGAAAACAAAAATCCCTAGGGAGACTCCCCTAGGTGTTCCCAGTGCATCAAGGTAGAGCCT
GCTGCAGGTTGAGTTTCTGGAATAGGTCAGGCTGTGTCTGCTTTGTTTGTTTGTCCGTTTCCTTGCTTTTCTGCTGCATGGGGACCCTCACGCC
TAATTTTCCTTCTCCGTGTATACATCTGCTTCCTTTAAGCTCCCTACAGTAGAGCAACGCTCCTAGATTCGACAGAGATCACTTGTGTGTCATG
TAATCTGTGACTAGTAAGCATTAACTATCCAAATGGGAGTTCGTGGACGGTGTCTGAGCCAGGGACTGCACAGAGCTCACGAGTTGCTGTAGGT
GTGCTCTAGAGAGAAGGTCACTGGGGAAAAAGCTTGAAAACAGCTGTGGCATGCAAGGGTGTGTGTAGGAACGGAGACCCAAGAGCACTGTGGG
GGAAGATGTGCTCAGGAGCCCCGAGGAAGTGGGCTCACAGGAATTTTCCAAAATCAAGAGATCCAGCCTTTAACTAGAAAGCTCCAGGTGGTAT
CATGAGTGACATTAGATGATGCCTGATACATGGTCTCTGAATGAATGACTGTTCAGGACTGTTTGTTTTTTTGTTTTTGTTTTTTTTTGAGATG
GAGTCTCGCCCTGTCACCAGGCTGGAATGCAGTGTCGTGATCTCAGCTCACTGCAGCCTCCGCCTCCTGTGTTCAAGCAATCCTCCTGCCTCAG
CCTCCCAAGTAGCTGGGATTACAGGCTTGCACCACCACGCCTGGATAATTTTTGTATTTTTCATAGAGACGGGGTTTCATCACGCTGGCCAGGA
TGGTCTCGATCTCGTTGACCTCGTGATCTGCCTGCCTCGGCCTCCCAAAGTGCTGGGATTACAGGCATGAGCCACTGTGCTGGGCCGGGACTGT
TTTGATGTTAGGCAGTACAGCCTAGTGGTCAGCACGTGGACTTTACTGGCTTCCCCACTGTGTTTCAACTTGGGTGTTGCTGACATTTAGGGCC
AGTTATTCTTTGCTGTGAGTGGCTTTCCTGTGCATGGTTGAACATTTAACAGCACCCCTGGCCTCTACTCAGGGGCTACTAGCACCCACCCTCC
TGGTTGTGACAACCAGAAATCTCTGAAGCCATTGCGTCATGTCCCCTGGGGGCAAATCGCCCCTGGTTGAGAACCACTGGCTTGGAGCTATCTG
TCCTTGAACAAGCTACTTGACCTGTCTAAGCCTGATTTTGCCCTTGTCTGTGGGAAGACAGAGGCACAATGAGGATTGATTACCTGAGACAAGA
CATAGGTTAGGAGTTGTTACTTTTTGCAGTGACACCAACACCATGGGGCAGACAATGGGGACTCTGTGAGTGCATAGCAAACAACAGCAAACAG
CAACAACAACCAGAGGTCTGCTTTTTTTGTTTGTCTAAATGGGTGAGCTTCCAAGGAGGAAACACAATCCTTATCCTTATTTCAGGGCCCCATA
CGGTTCTGTGGTCGTGAGAGGGTTATATGCGGCAGGCCCTCAGATTCCTGTTAGCATAGGAGAAACGAAATTAAAAATATGTACTGTAATTTGG
GCACTGCTTGTCATGGGAAAAGAAGTTCTCCCAACTCAGCCTTTCCAACTCTTTTGTCCAAACATTAATGGCATGCAAATATTTATGATTCATA
TGTGCCCCTTTTAATTCCCAATCGTATTTTAACAGTAAACACTTCATTTTAATAATGAAGCAGAATAATCCCTTTGTCCTGTCGCTGGGTTACA
CAGGCTGGCACCTGGCAGCATCACATTCTTCCTGAGTGATCATTGCTGCCTCCACCCAAGTTGAAGGGGTTTTGTAGCAGTGCCACGGCAGCTC
GCCTTGGACTACTTGAGGACTTCCGGATGTTCCGCTGGAGCCATTCTTAAGGTTGTGTCTCAGCATTTTCATAACTGCAAAAGGTGGCATCCAT
GCATATTTTTCTCTTTTGGCCACGTATAAACTCAGCACTGGTTCATCTGGCTGTCACTTGGAAGGGTTCTTACTACACTATTATCTTGCATTCT
GGCCAAATCCCTGAACTCTAGATTACACCGAACCCATTGTGTCCTATTTAATATTCCTTTGATTCGTAATGATGACATAGAGAGCACACACATC
TATTAGCGATGATTCTGTGAGTCCTAGAAAAGTAGCATAATGTAATATGAGAAGAGGGCACACCTGTAGGACTCAGATGTGCCAGCCTCAGTCT
CCCCATCTGTGTTCAATAGGTTTAAGAGTGCCTTTCATCTATGACTTATATCATCTATGTTAGGATGATAAAAGCGAGATGGGTGTGTATGTGC
TATACTTAGGAAGAAGAGCTAGGGCCCCTCTTCATGATGTTCTTCCTTCTACAGCCAGAATCCCTGAGGGAGGAGGGGAGAGAGCAAGCCAGAG
GCAGCATGGGTCTTGCTGAATTTGAAAGTTCGGAATGATGCTCTTTTTCAGGCCCCACTCCTCGATGGCAAGGAATTTATAATTCCTTCGTCCT
GCTAACGGCCTACTAAAATGAGAGTGCATGAGATAACAGCAGGCATTCCATAAGTATTCACAAGTAAAAGCCTATGTAATAATAATATAATTAC
AAACTTTTATTGGGCATTCACCCTGTGATGGAAATATAGTACCTATTCCTGTTTTATTAGAACTCTCCCTCTGCCCCACCCCCCAACGTCATTT
ACTACGCATTTCTTTCATTCATTCATTTATCAGATATACATTGTGCACCTACTCTGTGCCAGGAACTGGGCTAGCATGAGGGAAATACGACGGT
CAGCCAAAGAGAACCCACAGCCTTGGGGGCTAGTAAGGAACATGGCAATTAAGTAGGCCATCCCTGTTCTGAGTAGATAACACATGATAGATGG
GTCTGCCGGGAGCCAAGGCTCAGAGGTACAGAACCTGGTGAAGGTTCAGAGGGTCTCCCTGGACCACTTGTCATTCACTTGAGGACCTGCAGTG
ACAAGGAGACTAACCCGGTGGAGGCATGAAGAAGCATGTCCCAGCAGAGAAAGCAGAGTGAGGAAGGAGCACCTCGCCTGTTGGGAGCTGAGAG
ATGCTCCAGTGGCGGTGGTTGGGGAGTGATGGCGGGGAGCCTTACAGAGACATAGGGCGGGGATTGCCTTAACATCCACGTTGGGAATTTGGTC
TGATCCTAATGAAATGGAGAACCACTTATAACTAACTCCAGATACAAATTGTGGAAAGATACTTGGCTTACAGTTTAAAGAATGGAAAGGACAT
TTGGAGGGTGCGTGGGGAGCTTAGAGACCCATCCAGAGACTGAAAGCTCTCCTTTCTCCTTTCAACCAGCGCCAGCCATGTGTATATCCAGCTT
GCAAGGACCCCATTTGGTAGATGCCACTGTACCTACAAGTCAGCTCCCTCCTCAAGCTTGCCTCAAGCTTCCCTCTCTTCCCTCTCTACCTCCA
CACTCACCTGCCTTTCTTCCTACCTGCAGTGGCAAGTGCTGCAAGGCTGGTGAGCAGGAGCTGACGCCCCCCAAGCCTTCCTTCCCTGAAGAAT
GTCCATGCCTCAGGCCTCCCAGCTGCAGCTCTGTGTGGCCTTGGGCTGTGTGTTTGAGGCACCCGGCTAGGGGCACTATCGGGTCAAGGAGGAG
CCCAGGCCCAGGAGGTCACTTTGGTCACTTGCCCTGTGTCACCCCAGCCCAAACACACAGCCTGATGGATGAGTGAAGACTGGTAAATCTGGTG
GCTGTCCTTGGGGGAAGCACAGGGCAGCAGGTGTGGCCTTGGTCTCCAGGGGGCTGCGAATTCAGGGTTTTGGGGTGTTTTGGTTAAAACTTGG
TTGGAGGGTGCGTGGGGAGCTTAGAGACCCATCCAGAGACTGAAAGCTCTCCTTTCTCCTTTCAAGCAGCGCCAGGCATGTGTATATCCAGCTT
CATGAGAACATCATGGTCTTTTGTGGGATAGGTGATATTCTGCCAAATAAAGAGGCCCGCCCCTCCACTCCTTTTGCAAACCTGAGCTTTTCCC
CTGCCTGCAGTTGAGCCCAGCATACCTGTGCCTTGGAAAAGCAAGCTATGCCTTCCAAGTGAAATTCGACGTTGTAAAAGGACTGGCTGTCGGG
CTGAAAGTTTGCACAGTGATTCATTGATTTTTAGAGACAGGGTCTTACTCTGTCACCCAGGCTGGAGTGCAGTGGCGTGATCTCGGCTCACTGC
AACCTCCCCCTCCTGGGTTCAAGTCATTCTTGTGCCTCAGCCTCCCAACTAGCTGGGACTGCAAATGTGTGCCACCATGCCCAGCTAATTTATT
TTATTTTATTTTTTATTTTTATTTTATTTTATTTTATTTTTTATATTTTTGGTAGCAATGGGGTTTCACCAAGCTGCCCAGACTGGTCTTGAAC
TCCTGAGCTCTAGTGATCCTCCCGTCTTGGCCTCCCAAAGTGCTGGGATTACAGGCATGTGCCACCATGCCTGGCCTCATTTATCAAACTTTTC
TTTATTGCCTCAGAATAGAAGATACAAAGATGAATAAAATTCTGTCCCTGTCCTCAAACAACTCATTTTCATAGAACATCTCCAGAGAAAATGC
TTTGCATGTCTTCGGTTTTCTCCAAGTAAAACAGCTATTTTTCAAACAATCCATAATTCCAAACAAATGCCATTTCCATGGAAAACTGAGTTCT
ATATTCTAAGGACCTGATTTTCAGTCTGACTTTTGAGGGTAGTCCTGGTGCCAGTAGCTTAGGCCTCAAGTCTGCATTTTGCATGTGTGATGTG
AGTGCGATTGGATCAAGCAATCTGCAAATACAGGCACTTTGCACACAAGTATATACAGTACGCCGACATCTTAATTCAATTCAGCATCGAGGGT
CAGGAAATTATGTGCAAGTTGAAGGATTTGTATCCCGTCCAGAAATCCCCAAATAAATTAATTATACAACTCCACTGTCAAAAATAGTGGCAAA
TGTTTGGGTAAGGAAATGGAAGAGGAGCATAAGCATGAATTTTGGCTGTTAGGTTGTTCCAGTGGAAAACTGAGGAAATCAAAATTAAAGTACT
TCATAAAAAATACTCCAAATGCAGACTACTCCTGGAATCCGAGGGTACGTTAATTTTTATACTTAGTTATCCGGCAATTAAAATTAGACCCGAG
CCATCTATTCTAAAAGGAGAAATCTCCCTGCAACCTTTCAGGATCTTCTGGGATTTCCTGAATTTGCAAAGAGCAGAACCTGAGCCTTCACTGA
CTCTCTTGGTTTTTAAAAGCTGCGATGTTTTTCTTTTATTTTTCCTTTCTCACTCTTTGGGCGTTTCAAGACTCTGGGCACAACTGGCTTTCCT
GCAGACAAACACAAGACTGAAAAATGTTGCGATTGTGTTTCCTAAGCACTCTGAGCCCCCAGGGCAGACGGCTCCTTTGTGTCTGCAGCCTTCC
TTTGCGTGACAAAGGCCAAGTTTCACATCCCCTGAAACTGTCCCCAAAAGAAGAAGATGACTCATGCTTACAGACCTACACTAAGGAGAGTGAC
AGGAACAGAGTCTGCTGCAGAGCCAACTCAGGTAGACCGGGGATACCTGCAAACTAGACCTGAGGAGGCCGAGTCACAACAGGAGTCGGGGCTC
GCCCGTCAGACACGQGGTGTTATAGGAGGATTCTGCTCTGATGTGGCCTGGATGCACTTTTAAATATGTTCTTCACACCTAGGCAAGAAATGAC
CCCGAGCTGCATGTCTTTTTGCTTTGTAATCTCCTTGCCACCCTCAGCTCTTTGGGGAGCAGTGTGCACATTTGAAGCAATCCTGAGGTTTAGC
CACTTCCCAGATGTGGTGAGGAAGTGGAGAGTCCGCTGGGCCATGCGCAACTGCAGGTTCCACCCGTGGCTTTATTTGACCACAATTGGCTGTC
AAACTTCACCAGCCCCAGGGCCGCCAACGTCAAGACTAATCAAGCCTAGACCTGGGGCCTCCCCGCTGACTGGCCCTGGGGAGTGTGTCATGGC
AGCGAGTGTCCAGGAAGACTCACCCCTTGACCTTCTTCCCAACTAAATAAAGCAGTTAGTATTTTGCCCTGGCATTTGGATGGACACACTGGCG
TCGAGGGAGACAACACATGTTGCAGTCAGGAGCACTGGCCTGGCTTGTTTACATAGCATATCAGCCACTCACACTTGTGCCTTACAAAGGTCGC
CAGCTGCCGCGGCAGGAGTGAACTTGAACTTGAAGTTCAAGTTGCGTTTAGGCCTGGTAGCAGCCCCATCACCTGCTGAACACACACACACACA
CACACACACACACACACACACACACACACACGCACAAAACCCCTCCTCATGTCAGACGTCAGTGCCTACTTCATCCCCAGCTCCTTCTTACGGC
ATCTGTCACAGCTCTGGTCTTTGTGGAATCACCTTTATCATGTCTTTAATGATCATTCCACTCACATTTACTGAGACCTTTCCCTGTGCTCTGC
TTGTATATTCACCATTCCATTTAACCTGCAGAACAATGCTACGAGATGGAATGACTGTTATTAACCCCCTTTTACAGATGACAAAGCTGAGGCC
CCAGAGAGGTCAAGTAACTTGCCTGAGGTCACACAGCTAGCAAGTGGCAGAGCGAGGTTTTGAACCTAAGTAGGTTGCCTTGACAGTCCACACT
GAATGAATCACCACTGTGTTTCCACCTTCATGGGATTTTATATGGAATTCTTCTCTCTCACTGCTTCCAAGCCTACATCAAGACAAACCTCATC
TGCCCTGTTGTTTTTTGTTCTCACCTTCTTATTCAGTATGAGTCTCGTTTGGTGCTTCTAATGGTCTTCTAATCGTCCTCCTGCAACCCAAGCC
CTGGCTGGAACTGATGCTGCTGTGGTGGTCAGGGTTCAGTGAGCAGGAGCAAGGACTTGGTGTTTACCCCTTGTTTACCCCTTTCCTTGGCTTC
CTTCCATGCAGGCCACCCTGTGACCCCGTGGTCGCCCCCTCTGGAACCAAGAGCTGCCCACTTCCAGCAGCACAGACAGGATCACTCCTGGGGC
CATCTGTGCACTGACCAAAGCCTCTGCTGGCCTCACCAGACCAACGCCAGTGAGTCCTTCAGGGAGCCTTGGATCCTCCAGGCTGCCAACAGAA
ACACCGGCCCTCTCGGCAGCAGCCCCATCCTTCCACCCCTGCACTGGGTCCTGAAAAGCCCATTTTGGGGCCGTTGCTATTTAGCCAACCTGCC
CTCCCTTGCTCTCCTGTGATTTCTCACTATTCCGGCTGCAGCTCGCTGGGAGAAACACTTGAGAGTCTTTTGTGCTCCACACCCATGTACTTAA
AATACCAGGCCTATACGTCATTTCAATGAGGGAATTTGGCTCATAACACGTGTGCCCCGAGGCACAGATCCTACTTCTGCAGATGCTGGCCAAG
AAGGGCTGTGTCCCAGCCGCCATGGGGTGGGGCCACAGAGAGGGCAGGGCCACGTGGAGGGCAGAGCATGTGGCTCCTGTCAGGTGCGCCCGTT
GCTGACTGCAGCCCACTATCAAGGCTCTGCTGGGTCTCATTGGAAGAATACAAAGGACGTGTAAGGCAGTTTTCGTGCTGTGGAAAAATGTCAC
CTACACCTGTGAAGAAGTCATCACTGTGTCTGTAAGATAATAAGCAAATGTCCTTGTACCCAAAGCTGTAAACAAGAGCCCTTTGTGAGGGAGA
ACCACATGACTGGCCAAAACGCCCCACCTACTAGTGTGTGCTCGGTGGAAATGTCTTGTGACTGTGCTTAAAATGAAGGGCATCATTGGGAGGC
CAAGGTGGGCCGATCACAATGTCAGGAGTTCGAGACCACCATGGCTGACATAGTGAAACCCCATCTCTACTAAAAATACAAAAAATTAGCTGGG
TGTGGTGGTATGCACATGTAATCCCAGCTACTCGGGAGCCTGAGGCAGGAGAATCGCTTGAACCCGCCAGGCAGAGGTTGCAGTGAGCCGAGAT
CACGCCATTGCACTCCAGCCCAGGAGACACTGCAAGACTCCATCTCGAAAAAAAAAAAAAAAAAAAAAAGGATCGGCATGAAATAATGAATTCA
GATAATCACCATTCATTTCACAAATTATCACTTTGATTAAGTTTTACTCCTGATTATATAGGTTAGTCTGTGGTTTACCAGACGGGGTGTCATG
AGTGCTCAACTGCCAGAGGCCCAAACGCACCTCAGTAAGAAAATGCTTTTGAGCTATAACCCAGGTTGAGTACCATTGGTACATTAGAATCACA
GAGTCACATTTTACTTTTTCGGGCAGTGGTAGCTGTGGATAAAGTATCTCCAGTCCACATTTCTTGTACTCGTGCTATTGGCTTTGCGGGTGCA
GATTTATGACCTCAGGGATAATAACCGGAAGAACAGTGAGTAGAAAGCTCAGGGATATGAGTTTTGCTGTATATCAAAGCTGTCTGACTTTGCC
AAAATTACTTAACCTTTCTGGGCCTTAGCTTTGCTACCTATTCATCAAGAACAATAAAATCCATCTTGTTTATTTCATGAGATTGGTGTGAGGA
CCAAATCAAATAGTATATGGGAAGGTGTTTAAAAAGTTGTAAGTTCTACACGACTTAAAAATGCCAGTATTATGAATGCAACCATTCTTTGTTG
TCATTTGGGTAGTCGTGGATAGCGTGGTGGTAGGAGAGCCACTATCGGAGCAAGACTGTTCCAGAGGGTAAAACACACGCGTGCCTGTAGAGCA
GTTGTCACTGGTAGAGCCATGATGGGAGCTCTTACTACATTGCTATTTGTACTGAGTTAAATACTGTTCTCCAAAAAGTGTATGTCCACCTAGA
ACCTTAAAATATGGCTTAAGGTCTTTACACGTGTAATCGAGATAAAGTCATACTGGACTTGGGTGGGCCATAAGTCCAATGACTGATGTCCTTC
TAAGAAGAGGGAGGGGACACCCAGTAGGAAGACAGTCATGTGAAGATGGAGGCAGAGGCTAGAGTGCTGCAGCTGCAAGTCAGGAAACGCCAAG
AAATGCTCGCCACCATCCGAAGCGAGAAAGAGGCAAGGATCCTCCCTAGAGCCCTCAGGGGGAGTGTGGCCCTGCTGCCACCTTGATTTGGGAC
TTCTGACTTCCAGAATTGTGAAAGTCTTAATTTCTATTGTTGTAAGCCACCCAGTTTGTGATAATTTATTACAGCAGCCCCAGGAAACAAATAC
AGTATTTTTATGAAGTCACTGAGCTGCTAGAGTTGTAGGCAACAGAAGAATCACACGTGGACCTGCCCGCCTGCACATGGCTATGGGCATCTCA
GGCCAGCTGCATTCTTGGAAGATGAAATCAGAGAGTTGAAAGGGACATTGCATGCATCCCATGAACCCCCCTCATTTTGCTGATGAGGAAACTG
AGATCTGGAGAGGTAAAATCGTTTGTATTGCATCCCCAAACTTGTAACTCTTTTGCTGAAACAACCACCTCAGTCTCTCTCTCTCTCTCTCTCA
TACACCCACATGCACCCCTAGACCTACTCAGGACTGGGACAATGGCATTATCTATTGTTGTGTCCTCAAGGGACTTTTTTTTTTTTAATTTTAA
ATACCCCCTCTCTCCTAAAGCCCCTTCCCACTTGCACCCTTTCCCACCTTTCTGTGAGGGCTACATGGGTATATAGAAAGGGGTCCCCACCTTC
TGGGAGACTAGGTCTACACAGCCGCAAACTGCATTCAGTATGACACCCTAAATAATCAAAGGTGAAGGATGATTTATGAGGCAGCCCAAGCAGC
CATAAAGCAAAATGTACTCATCAAGGAAGGCTGCCTGGAAAACAAGTGACTGAGTGACTGCCCGTTCCCAGATGCTGAAGGGCAGGAAGACTCG
GGTGAACGGTCCAGCAAGGAGCCTTGGATTGGCGAACTGGCTGGAGGAAGGAAGACCTCAGCATCTCAATGCCATGGCTCATGGGTAGAACTCG
GAGGGTGCACACTCGGAGTCAGGTATGCGGAGGAGAGGGGGTGAGTGAGAAATTACTTGGGGCAGQGTCTTATTAGTCAGAAGATCCTAAAAGC
AGGTTCTGGAGTTCAGTCTTGATGATACAGGTGATAACCTTCATGGGTTCTTGACAGAGAGGGAACAGGCTGACCATGAGGCTTGTAGGACAAT
CTGTTAGTCAGCTCTGGAACATGTGGATGTGGCTGCTCCCCGTCTGTGGCTGTCATTGCTCCTGGATCTGTTCTTGGAAACCCAGAGACTCCCC
AGTGAGATGGGCAGTTGCCCCCACGACCTGCCTCCCTCTGCGGCAGCATCTTCCTCCACATCACATCACATACCCTGCTTGCTGGGCCTGGCCA
AGGCCTCTTCTACCCAGTCCTCTCCTTCAGCTAGATGCCACGGTAGGAAAGCATCCAGAGTGTCGCCTGATAACATCTGAACTGATGTCCTGGG
CTGGCAAATCCCCAAAGGCCAACAAGACTCGTCTCTATGTTCTGGGGACAGCCCAGTTGATGGGAAACATGGATTTGGAGCCAGAGTCTCCTCG
CATCCCTGATCCTCTGCTCCATGGAAGTCACTCCCCAGAAGCATCCTCACCTCGTTTATTCCACAGGTCAGCTGAGACTTTAGTTATAAAGGCA
CAATCGGCAGAGCAGCCTTCATCATCTAATTGTTACTTGCTATTGCTCTTTTTATTATAGACTTTAATACAGATAGAATCCACATCTTTGGGGA
GTGTTCGCTGGAGATTTCTCACGATTACCAATCGTGGCTTGCTGTCGCCAAAGATTATATTTTCATTGAGCATTTTTCTGTGCCCTTCCTTTGT
ATTAAGACACGCATGACATTTGTTTTTCTGTGGCCTTCCTTTGTATTAAGACATGTGTGACAATTGAAAATATTTTCCTTTCATCTGTCTTTGA
TTTACTTTATGCTACATGATTAAGAGGAGAAACTATGTTGATTAAAGAACTACTGTATTGATGTAGGAAGATTGAACATCAGACTTAATCAAAT
GTTAGGGTCCTATTATAGACCCGTGAGAAACAGAGAAGAGAAATAAAGCAGACAACATACACACACACACACACACACACACGCACAGACACAC
CCTGTTGAATACCGTGTGCCAGATAACTGTTTTAACAGGGTTACTCACGGTAAATCATTTAATCTCCCCTAAAACTCTCTAAGGTAGGAAAATT
TGCATGTAACCATCTTCCTGGTAAGGATACTGAGGAACATAGAGGTGTCTAGTGACTTGCCAAAGGTCAGCTAGCCAGATTTATCTTCTGAATC
ACTTTACCAACAAGAAATGTTTTTAGATTAGTAAATCTATGCAATGAAGATTTTCTCACCTTTTATAAATATATGTAAAATGTGTAATGGAAAT
TTTGTGCATTTTTTATTACTAGTACTTATTGAATCTCAGTCTGTCAGTTACATATACTTAAGTCCATTTATGAGTCATCAATTTTATTCTGACT
GATCCCATGAGTACCTCTAATAACATCTCGCATTTTCAAATAATATTTAAAATCAGCCAGACTTCGTCTTTTAAAAATATAGACAGAATGATTC
TAATTTAGCCTGTTTTCAAACTTAGAAATTCTATCTTTCCTGATCTTCAAAGGTACAAAGTAACAAGAGAAAGATGGATATGGTTTCTTCTACT
CTTATTTGCCCTCAATTATAAATAATTGGGAAAGATGGGAGCAGGGAAGTTGGGAACCAGAAGTGGAGAGCTGCCAATCTCACTCTATGGTTTC
CGGAAATCCTGACTTAACAGAAAATAAATTCTAATCTTTCTTGATCTTGTATTGTTTATTGATGATTACATTAAAGCCATATAACTATGTGAAG
TGTTACACTTAGGCAACTATCATAGGTGTGTATTCCAGAATTGAAGCAAAGCCAAAATTCCGGGAGTGTTGTCAAAACTGGTAACTTGTGCTGA
AGGGCTCGACAGCATAAATTAATCATTGGTTATTCAACAGATATGTTCAGGCCACCAACCTCATTCTGTTTTGTTCTCTATCGTCTCCCCACAG
GGAAAAGCTTCACTCTGACCATCACTGTCTTCACAAACCCACCGCAAGTCGCCACCTACCACAGAGCCATCAAAATCACAGTGGATGGGCCCCG
AGAACCTCGAAGTAAGTGCATCCACTTGGGGCTGGTACACCCTCCAGGCTGGTACACCCTCCAGGCTGGTATACTCAGGGACCATGTCTCAGAT
TCCTTGGGTTCAACCTTTCCCCCAGGGGGATGTCACCCCCCCGTAAAGCCAAAAAGTTGCATTGTCAATACTCTCGCTACTCAAAGCTTGTTTC
TGTGCACTGAGCTCTTTTTGAAGCATCCTTTTAGAGGGCAAAAGGAAATTAGGCTGACTGAATCATATGTTAGAACCATTCAGGGCAGCATGAA
ACTAAGCACTGCTTCCTCAATATACACTAAAGTTCAACAGGATCACAAACAAATAGTAAATGGAAGATTGGCCAGCCACAGAGGCAGCCCAAAG
AGGTCTGAGGGTCAGAGTGGACCACAAGGGAGTAATTACCGTCTATGCTGGCCTTTTATTAATTCATGAGGGCCAGAGATGAAAACTATGAAAA
TGACCCTTTTCTTTCTATGGCACAGATCAAAATCTTATTACAGGCATTTTTAAGATAGAGAGTGGGAGGGTAGAAGAATGTCTGGGGAAAACCA
GTAAATATAGAAAGATAGTACCAAAAAATAACTCCTGTGAGGGAAGCTTTTAAAAAAATGAGTGAGGTTGTTTAGTCTGCAGAAAAGATGTCTT
CACAGTGACTTAATGACTATCTTTGAGTAAACACCAGAGTAACGCTCTTTTCCAGAACCACACCCTTCCCCAGGATCTGAGGCTGGGTTACAGT
TCCACGTCTGTGGAACTGCCCAGCACCCCCGCCCAGCATCCACATGTCTCCAAACCGTATCAGGTCCCTGGTCGAAAATTAATATACTCTCTTC
TTGGAGTGAGGGATATTGAAAAACAGGGCTTCCTACTGTCCCCAAAAGCCAAGATGCAGGGAGGTCAGGTTCTAAAATGAAGTCGTTAAGAATT
TGGGCTTTATGGGCAAGCCAGTGGAATTCTTAAGCCTTCAGTGAGCTACAGAGCCTTCCTGAACCTCACTTTCCTCTTCTGTAAAATGGGGATA
ATAATGAGAGCTTCTTTATAAGATCAAGCCAAATGATGCTGTGTGCCTGATGCAGAGAATGAAGGCAGGAAATGTTAGTGATCCCCAGTAGGGC
ATCCCTGCTTTCATCCCTGGCTACTGCTCCAGTCCCCTCTAGATGACCTTCCCTGCATGGTGGTTTCTAGGTGGGGTTTTAAGGTAGTTCTCTG
CAGGCTATCCTGTGATCACGGCAGCTTTTATGGCAGCCTTCCCTGTGGTCCTGGCCACAACCTCATGGCATCTGCGTCACCTTCTGGAACCATT
CCCCAAGCTGCATCAACTTTCCCAAAGTCCTTGAGTGGATGGAGCTCTGCAAATAATCACCATGCCCACCAGGGGCAGGCCACCAGGATATGGT
GTTCCTCCTCTCCTTTTTCCCTTCCTTCCATTTGCTGTGCTCGGTGACTCCCTCCAGATCTGACTGCATTTTTCCCCAGAGGGCCCTCTCAGCT
TTTTCTGATCGAGACTTTCTGGCCATGCCTAGACTTGGGAGACTGTGGGACTTGATAACAGACCCTGATTCTTCTTTATGAGTGAAAAGCAGGC
CATAGACACAGACAGGACCACACAGGTCTAAGGAATGACTGTGAGCTCTTCTTAGGTTTCCAGAGTAGGAATGTCAACCTGGACATTCCTACGT
TAATGTAACCCTGCATTAAACAGAACATAAACACCAAAGCAAGTTTGTTCTCCACAGAGGACCAAAGAGGAAGCAGGCTTGAATTATGGCAAGC
CCCCTGGCTGGGAGGGAGGCAGTTGTTAGGTATAAGAAAATCGACTTTGAGGGTGACTAAACTTAGAGAAATGCTCCTTCAGGAGGCTGTAGAA
CCACTGTATTTAGAACTAGAAGCAACAGCCGTCTCTGGCACAGTTGAAATGCCGACCAGCCTGAAGAAGCCCAAGCCAGGTGACCCTCGGTATA
CCCTCTACTTCTCTTATTTCCTCATGCATAAAGAAGGCTGGGTTTTGCCTCACTGCCAGCATTTCCTGTAACATTCAAAGAAATAACAAAGTTA
TTGAGCTTCAGAAAACAAATGGTCGCGACTCTGACTTTTAAGAATTGGCAGTCAGAGGGGAAAAAAGATGTCTATGCGATTGTAACGGTCTGGC
TGTATATACAGTTTACTCTGCCCCTGCTGTTTTACAACTTAAGAAAACAGTTGTTATATGGAGAAGCACCCTCTGAGCAGACAGAAGGGATTTG
CGCCAAAGAAGCATCCATTTTTACACAGTCTTCCCTGCCAAATGGAATTTGGGGCCTGGGCAAAAACTAAATGCCTGGAATAAGTATAAGTATG
ATATATAGAGGTGCAATAGCCCCAGGTAGAAAGTCTCACATTTTACAAGGGACTTCTAGAAAAAAGCCAAGTGCATGCAGACTCCCAGGTTGAC
TTGGGTAATGCTGACTTTTCTATTTGCCCTGTGAAGCAAGAGGGGCCATCACTGCTAATTTTAGTATCCTGGTTTCTTATGCTTTGAACGTCTT
CATTCAGGGTGGGAACTTTCTATAGTAAGCTTTGCTCACAGTTGCTTTCCAGTGTAACATAAAGAGAGGACACTGCATGTAAATCAGGATGCCC
AGATGTTGCCAGATCTCTGGATGAATAAGACCTCCCGAGTCCCCTCCATGATTATGGTGCAATGAATTCTGGGAAATCCTGGCAAAATGGCCCT
GGCATTGCCAAGGTCAAATTGAATCATTTTGGAAAATTCTCTAGTTTCATAAAATCAAAGACAATGAGAGCTAGCAGGGGCCTGAGAAGAGCCT
TTAGGATCATCACCCCATAGGTAAGGGAAAATAGCCCCAGAAAAATGAAACGGTTTGCCTAGCTTTAAAAGAGATAGTTGATGATGAAGCGTTA
ACAGAATGAGGAGTTCCTGATGTCTACTTCTCTGCTGTCCTTCATTTATCTACCTGTCCATTCATCTGTTCATCTCTCTATCCATACCATACTG
TCTGTACATCATCTATTTCTTTCTTATAGACTCTTTGACCTGCCCTCGGTATCCTCACATTTAAAATGAGGACTTGGCTTATATGATTGGCTGG
CATATTTGGCTCATCTCCAAGACTGTCTATTATAGAAGGGCAAATGGTACTGCCTCATTGCAGGTGTGGAGCCTTTGACTTGAGGTATGCCTGC
AAGCTTAACTAAATAAAGCAGTTATCTGCCATTTGGAGGGACAAACTGCCATTCAACCTGCCATTCCGCTGACTAAGTTAAGGACTGACAATGA
GAAGCTGTGCCACAGCTCCTCTGTTTTTCATGCCGACGGGTAAACTTGATGCATCCCTCCCCTCTTCCAGCGTTTTCAGTGGTGAAGCTGTAGC
CAAAAGTATACGCCAAAGGGAATTTAAAGTGTTTTTCGTTTCAGATGGTTTCTTGCTCTTTTACAGTAATGGACCTCCCCCTTAGAAAATCAAA
ACTTGTTTTTCTCATATTCCATAGGATTTTAAGAAATATCTTAGAGTAATATAGAAATTATTAATTGTTGAAAAATTAACTTTATCATGGAGCA
AGAGCACCATCAAAAGGTATCCTCTGCCTGGAATCGGTTCTTACCAGCACCAAGGTGGGCTCTGTTTTCGGTTAGCACGTAGAGCACTTAGTAG
CTTGTTGATATACTCAGAGTGTCTCCTCCTTAAGCACAAAAGATAGAGCCAAAGGATTTTTCAAGAAAAGGATATTTGACAGTTTTTTCTTTGT
TTGTTTGTTTGTTTTTGAGACAGAGTCTCACTCTGTTGCCCAGGCTGGGGTGCAGTGGCACAATCTCGGCTCACTGCAACCTCTGCCCCACTCC
CAGGTTCAAGCGATTCTCCCGCCTCAGCCTCTGGAGGAGCTGGGACTACAGATGCCCACAACCACTCCCAGCTAATTTTTGTATTTTTAGTAGA
GACGGGGTTTCACCATGTTGGCCAGCCTGGTCTCCAACTCCTGACCTCAAGTGATCTGCCCACCTTCGCTTCCCAAAGTGCTGGGATTGCACGC
ATGAGCTACTACGAATGGCTGAGAGTTGTTTTTTTAATGTATGTTTTATATTTCTAGGATCTAATGAAAAACTCCAGCTCTCAGCTCACAAATC
TGTAATTTAGGTGTCTCTATGCTAATATTGAGATCCCAAAGCATCTTCTCTACAGAGATTTCATTAAATAATTTCAATCCAGGAATTGGTTCAA
TGCCATGGAGGAAGATATTTTGCCAAGGAGAGGTTGAGGAGAGTGAGCAGTGCGGACCCTAACTGACCCGTCTTGGGGCGCTGGTCCTGGTCGT
AATGGATTGGGCAGCCTTTACCTGTCTGGTCAGGTGTTTGTTACGTCTGGAACATTGAGAAACTAAATTAAAAATCAAAAGGTTAATTAACGTA
TTGGCCATTTCTGAGATAACTTCTGTCTGTCCCCAGGTTAAAGGAGTCTTTCACGTGACTGTGTTCTGCTTGAGCTCTTGATTTTTTTGTTTGT
TTGTTTAATTAAATATAGTTATTTCTATTTTAAAAGTGAAAGAGCTGGCTGTAATTTTGGAAAGGTGTAACCCAGCAGATGCCGGGAAGTACAA
ACCTGGAGGGAAGGACAGGCCTCTGCAGCTGCCCCCCTCCTCACAGGCAAGGATGACCATGTCTAGAGCTCACGGGATTCCTTTTGGGTGGAGG
CAGCGTTGCCTCTGCTCTCTGGGCAGCCCGTCCTTGAGGACCACGCTGTTAACGTACACTCTTGTGATGTGGTCTGTTTAAGGCTGCGGCTTCT
AGCATCCATACATCACGTGACATCTGGGATACTGGCTTAGAGGGCAGCTAGGGAGTAAGGTAAAATGTGCAGCTTTCTTTTTTCTTTTTTTTTT
TCTTTTTTTTTTGACGTGAAATCTCACTCTGTTGCCCATGCTGGAGTGCACTGGTGCGATCTTGGCTCACTGCAACCTCCGCCTTCCACGTTCT
AGCGAGTCTCCTGCCTCAGCCTCCTGAGTAGTTGGGATTATAGCCATCCACCACCATGCCCGGCTAATTTTTTAATTTTTTTCATAGAGATGGT
AAGGTGGAGCCATTACTTACAAACTGTAGAGTGCTTCCTGAGGCACTAGCTTTCTTTCTGTCTGATTTTTGTTTTTAGAGGGAGTTATAGTTAA
AATAGGTTGATCAAAGGGCCTAATCTTCACTCAGCTACTTTTTAAAGTACTTGGTTGAGATCATGTCATATGAGTATAATTCTTCTATCACTTT
GAGTGTGTATAGCAAACCTATTATATATCTGAACATGAGAAATAGGATCTTTATATATGGAGAATGTGCTTCAAGTGAAGAAGAGAAATGCATG
AAAAGTACCTATGGGCCAGGCGCAGTGGCTCCCGCCTGTAATCCCAGCACTTTGGGAGGCCAATGCGGGCGGCTCACTTGAGGTCAGGAGTTGG
AGACCAGCCTGGCCAACACAGGAAAACCCCATCTCTACCAGAAAATACAAAAATCGGCCTGGTCTGGTGGTACACGCCTGTAGTCCCAGCTCCT
CAGGAGGCTGAGGCAGGAGAATCGGTTGAACCCAGGAGGCGCAAGTTGCAGTGAGCCCAGATGGTGCCACTGTACTCCAGCCTGGGTGACAGAG
TGAGACCTTGTCTCAAAAAAAAAAAAAAATGAAGGAAGGAAGGGAAGGAAGGGAAGGAACCAACCTGTAAACTGGTGGTGATAATGGACCTAAA
AATAATTGCTCTTAGGATTCAGTAACACCTAGTTCACATTGTTATTTTCTCTTAATCATAAAAATACTCGATAATAGCAATAGCTAACATTAAA
TAGTGTTATTTGCCCAACACTGTTCACAGTGCTTCACGCATATTAATTTATATAATCTTCAAAAGAACAGGCAGGGAAATTGAAGAAAAAATAA
ATCGTATACCTTGCCCAAACTCACGCAACTAGTAAGCCACCATGTAAACTAAGGCGTCCAGATGCTGGCACGCACCGATGCCACCGTCTGTTTT
TAAGAACTGCACTGGCCGCTTTTTGAGCTTCATGGTAATGTAATTCCTCTATTTTTTATTGGGACAGTTTATCAGTAAAGTTCTGGGTCACATA
CAGTTTTCTTCAAGAAAGCAGAGCCCTGTTGCTTTGGCATTCATCATTTTTTTTGTACTGACAGGAGGGAATCACTGTTTTCCCCGGCAGGGCT
GCAGGAAGTACCCTCCCCTCCCACACCTCCCCAGAGTTCATAATACACAGCCATGGCCATGCTCGTCACAGGTGCCACCAGTTGGTTCTGCTTC
TGTCACCAGGGTCCACTTGGGGACCTGGCATGAAGGGGGACCTGACCCCGGGGTGTTGTACAACATAGACCTCTTCTTCCGGTGTTGGCCTTCA
CGAAAGATGGGTAAAGTGGAGTGACCTGCAGAGTAGTGGTCCAAGTCTGGGTCCCCCTTTAACAATGGGTTTCGTGGAGGGCCCGTGTGCACAG
CACGTAGCCGCCGGGGGGATGAAAAGATGTGTCACTCTTTGCTCTTGAATGATTATGAAAATGTCAGATGGTTTTATCGATCTGTGATCTCAGG
ATTGTATTACCAATCCACAATCGCATTGCTCACTGGGAATTGCTTCAAGCGTATTTTCCAGGTAGTGAATGATGAGCTACTTAATTTCCTCTTC
CCTTCAATTCATCAGATTTGGACTTGGACTTTAGTTCTTTCTAGATGCAATCCTGATCCACACACGAATTAGAAAGTAATTCTTCCTCCCCTCC
CACCTAAAGCAGGAGCAAGGCAGAGGCTCAAAGAGGAGGAAAATGCTGTCGTGACTCACATCCCCTCCTCCCCACTTTGAAAGGCATTTCAGGC
CAAGCTGAAAGTTCTGGGGGAACTTCCCCCTGGTACTTTTAATTCCTTCTGTGTTAAGCTCCTCAACTACCTGATTACTTGCCAAAGGCAGTTT
TTAAAGCATTAATAATAATCGTGCCTAAATGTTTCCAGCGAATTTTAGAATCTCTTTCTGTTGAATATTTTTAGTAGTATCAAACCAGTCCAAG
AAGAAGAATTTTACATGCATACAGACTTCCTTGACCCTAGAGTGGAGGTTAAGGTCTGAAAGGGCCCCTGGGGTGAATGGGAACCTAAGAGGAC
CGTGAAGCGAATGGGCTTCTGTCCCAGAAGCGGCAGTAACCCACCTGGTGCAGCTCTGAGTGAGCTGGCCTCGCCTGTGTATTCTTGGTAACTT
TTCCTGTCTTATACAGAAACCACTGAGGTTTTTTGTGTGTGGTTGTTGTTTTGTTTTGTTTTGTTTTGTTTTGTTTTTTGAGACAGAGTCTTGC
TCTTTCGCCCACGCTGGAGTGCAGTGGCGCCATCTCACCTCACTGCAACCTCCACCTCCTGGGTTCAAGCAATTTTCCTGCCTCAGCCTCCCGA
GTAGCTGCGACTACAGGTGTGTGCCACCACGCCCCGCTAATTTTTTGTATTTTTACTAGAGATGGGGTTTCACTGTGTTAGCCAGGATGGTCTC
CATCTCCTGACCTCAGCTCATCCACCCGCCTTGGCCTCCCAAAGTGCTGGGATTAAAGGCATGAGCCACTGTGCTTGGCCACCACTGAGATTTT
CTAAGATCGCATTTGTATTGCTATAGAAAGAAAACAAAATAAGAGTTCCTTCCACCTAATGTGAAAACCCAAAGAGATGATCTAGGCACCTTCT
CAATCCAATGTGGGAGCAGAGAAGAGGGAAGAAACTTACACCATTACATTGAATTTCTCTTTCCGTCTCGACCCTCGCAAAACCATATCACTAA
TTTGAATGCCACACCAGTGAGGTCAGGATCTAAACTAATATATTGCAGTCCTTGTTAGAATTCATTGTATGACATGCCATAAGAAAAAAAAAAC
CCTGTCTGCATTTAGCCTTCTGTTTCTTAAGCTTCAAACATGTAAAAAGTTTTCTCGATCCTGAATTTAGGGACCTGGGTTTTCACACTGGTGC
CACCAGGCCAGTGGGTGTGAACTTGAGCAATTATCTTAAATACTTTCGTCTTCATTTTCCTCTCCTGTTGAACCAGGCAATGGGACGAGATAAT
CTCTAAATTAGTTCTCATGCAAAAAGGGTAACAAAATATGGTCAGCAAAATAAAGCCGTAAGTTCGTTCCCGTAAACTGGCCCCTTAGAGGCCG
AGGCCTCCAGGCCTTCCCTGGGCAGTGCCATCCTAAATCACATCAGCCAAATCCACATGCACTCAGGTGGTACATGAGATAGACGTCGTTTTTT
TGCTGTAAAGTTTTTAAAGGGCTTTCTAATTTCTTCTGAAATTCTCTGGCTGTAAGTGCCTCCCCTGCCTTCTCTGCCACCAACATGTAATCAA
GACTAGGGGTTGAGGTCTCCATTTTGGCACTTGCTAACACTATGAGAGCCGATTCCTGGGGGAGCAGCTGGTCTGACTGAGCCTCTCCAAAACG
GGGACAGTTGGTAGTACCACCGTCACATCACTGGAAGGCTGAATGCACCAGCCTCTGTGGCTTAGAACCATGCCTGGCACACAATACATACTCA
AAAGAGGACAGGTATAATTCAGATTTAGTCTTGAAATCCTTCTGGAAATCAGAAAACTTTAACCAAAAAAGATCTGTGGCTCCTAAATTTAAAA
ATTCATGAAGTTGCCATAATGGTACACTTTTTTTTTTACATTTAATTAAACATTTATAAATTTTGATTTATTTTTCTGGATTTCGTTTTGTGTT
GAGACCCAGTAAAATTTCTTTTCTAGTATAGCATGGGCCCTCGAAACACTCTGGGCACTGTTCCTAAGTCACTAACGGGTGAAATGGCCTTGCC
CCCAAATGTTGCACTTTGTGAAAAAGTCAGAAAGAAAAGTCACGTGTGGGTGACATCAAACATCAGTGCTTAAGATCTAACACATGGGTCCTGT
TCAGTAGAGGCATGACACAGCTGCACAAGTCTCCACACTGTCCTAGAAACGTCACGTGTAATGTAGCAGGTGTCTTGCGGTCGGAATTCTTGTA
CTAGGCAAATGCAGTGTGTCTCCCAAGCGTGCCTCGGATCTCTCCCCGCAGCCAAGCTTTGCAAGCACATAAACATGGCCGGCCACCCACTGAG
TTTCCTAAATCAGTGACCAGCACTGGAGACCCTTCCATGTGTGCAACCCAGCTCCGCCTGCTGCCTGTGTCCTTTGGGGGTCTGATGGGGCAGC
CCCAGTCTGAGATGGTCAGGCCTTGTCATTGGGTGCACGTCTCCCATGGGGATGGATGGACCCACAGCTTTCAGATGGATTGCGGTTGGCAGTC
ACAGCATCCCAGCACATTAAAATACATCAACTCCTGTGCTTTCAGGTAGATTTAGAATTTTTCTCATACCCTAGCCACTTTCCATTTTTCATTT
TGAAAAATTTCTCCTTGAGTTTTCTCAAAGGCTTCAGTTTTTTTTGTTGTTGTTGTTTTTTCCTGTAAGCCATGTAACTAGGAAAGGCATTAGT
AACAATTTGATACACTGGGTCTCATGGAACTCCACTACTACCACCAAAATAATAATAGTAATAATAATAATAATAATTCCAGTTTAGCTTAAAT
AAGTAAAAGCTACCCCCTTACACCATATCTGCATTTAAATTCATTTGAGCTGATAGGCAATTTCTGTGTTCTCCTGTTCATCGCATATTCTTTT
CCTTTAAAGGCAACTTTTAGAGAAGGTTTCCTTTTTTTTGCTATCAAAGTATTAATTATGAAAAGTTGGCAATACAGAAAGGGTAAATTAGGTA
TAATCTATTACCGTACATTATGGCTATATTATCAGACATATCACAAGCCAGTGACATTTAGTTGGATGTGAAATTCCTTTTTTATAGTATGATT
GACACATGGTATATTTTCATCAACTTCTATGTGTAAATTATGCCGTGTCACTGGTAAAATTTGCTGTCATGAATAGCCAAATATTCTGGATTCA
CATTCATAGGTGAAAGCCAAGCTAATATATATATATATAATATATATTATATATATACTATATATTATAATATATACTATATATATAATATATA
TACTATATAATATATTATATATAGTATAATATATTATATATACTATGTATATAGTATATATAATATATTATATATACTATATATAATATATATA
GTATATATAATATATATATAATATATATTATATATTTTATATATAATATATATTATATATATATTATATATATATATATGTACCCAAAGCAAAG
TTCTTTTCTGTTTGCCTTTTCAAAAAGTAACACAATGGAATCAATCTGCCAACTCTCAAGACCCCTAGAAAGTTATTTAAAGGCAGTACATTGG
TTTGGTACTTTCATGATATACATATACTTAGTTTCCATTAAAGCAATTATTGACAAAAACCCAGACTGCTATTGAAATCTTTCAGTGTACTTCC
ATATACTGAAATGTCCAACATTTGATACTTGTTTGATTTTGTATTTGTTTTTGTTCACCATGGCTTAAAAGTCATATTCTGATTATTTAAAATA
AAGAGTAAACATTTGAAATAAGTTCCTAGATACTACATAGATTTATGGCCCTTTGAATATTTTCCTGGTACCACCTTATTTTCACTTACCTTTG
AAGTGGTTGTATGATATTTCCAGTTCATTCATTTGAAAGAGCTAGGAGAAATATGCGGAGCCACGTCCCACGCGCCTCTCGGACACTACTCGCC
ACGCATAGCCCAGTGTTTCAAAAGTGAACCAGGGGGCTGGGCGCGGTAGCTCACGCCTGTAATCCCAGCACTTTGGAAGCCCGAGGTGGGCCGA
TTACCTGAGGTCAGGAGTTCGAGACCAGCCTGGCCAACATGGTGAAACCCCGTCTCTACTAAAAATACAAAAATTAGCCGGGCATGGTGGCAGG
CGCCTATAATCCCAGCTACTCGGGATGCTGAGGCAGGAGAATCACTTGAACTCGAGAGGCAGAGGTTGCTGTGAGCCGACATTGCTCCACTGCA
CTCCAGCCTGGGTGACAGAGTGAGACTCTGTCTCAAAAAATAAAGTGAACCAGGGTGCTCAGGTAGCCTCGGTGAGCGAGAAAGGAGTTTGGAG
CTTCAAGTCTAAAACTGCGTTTCACAGTTTTCCCATCTGTAGAATGGACTGCATGATACCTCTCTTGCTAGGGGATGTGAGGATTAAAAGAGAT
AGCCTGAAAGGCGAGCTGGAGCAGTGGCTCATGGCGAAGCTCCAGAGCTGGCTTCCTGATTCAGCCTGGCCTCCCCTTCGGGATCAGATTAACC
TCCCCACCCTCAGCCTCCTTGGCTATAAAACCAGGAGTATGATATCCCTCTGTTGGGGATTATTGTCCGGATTGAATAAAATAATTCATGTTAG
GGCATAGCACCGTCCTGGGCACAGAAGTGTTCCTTAAATATTAGCTGCTATTATTAAAGCACATGCTCTAGACGGGTAGGCCCCTTGACCATCT
CCCAAAGACCTTATGAGAGGAAACAAATGGAATTCTGTTCAAGTGGGGGCCAAATATTTACCATTGCCAAGTACTTAAAAAAAAAGAAGAAAAA
AAAAAGCTTGGCAGATTTGGCCCTCACAATACAGTACAATAAGGGCTTTTTCTCTTTTCCTTCCATACTACCTATTATTAATTCCAATTACTGC
ATCACTGAACTGGCTTCAACAAATGTATTCCTCATTGAAACATAACCCTGTCAACCATGTTGCCCTACTTCCTTGAGGGAGAGGTGATCTTTTT
CCAATTAAAGGTTTTATCAAAATGCAGTTCTGCTTCTTAACATGAAGCACCTATGCTGTTGTCTCACCTGCAAAGCTCTCAGCATCTAGAAGCT
TCCCCTGGGTCATCTATAAGATGGGAATAATACTATGAGTTCTGCCTACTGCACAGGGTTCTTGAGGACACATTGAGATAATATATATCAATGT
GCTTTTTAAGTAGTCTGTCAATTAGATAATATAAATATAATTAGATAATTAAAAAATAGAAAATCTATTATTGTTTTGCATGTACTATATTAAA
ATCTTAGGGATTTTAAATAAAATTAATTGATTTATTGAAAGTAGATATTATCTCGTCGTGTCTTTCAGGATTGGTGAGAAGAAATTAAAGAGAA
AAAATTTAAACATTTAAATCAGACCCCACTGTGAATCCATACTTTTAGAAATTTTACCTTTTTCCATTCACATATCTTAATCTTTTCTCTACCT
ACAACTAGATACCCTTTTTAAATGTTCGAAACAAAAGAAGTAACCTTGGCAAATAGTTGAGAACTGAGTTTTATATGAATATACTTTTCTCTTG
CAAGACCAAATACAGTTTTTCTTCTAAAACTCTGTTATTCCATTTTCTTTCAATGAAATGCTCACAGAACCAGAAGGCTTGGGAAGAGGCTCAG
AGCACCTAAGAGTTGCTGAGTCCGTATTCCTTTTTTCCCCTCTGGCTTTTTATATATGCTGCTCGCCTGAGATTTCTGGATTTGATTTCAGGGA
GGTGAAATGTTACACCCCTGAGTCTCAGTGCCAGTTTCCCAGGACGAGTCAAAAACTGCAGCTGCTTGGAGGTGAGAATCTTCCAAGGCTCCAG
AGGCCAGCTCGCTGTCCTGTTCATTTGCTGACATGCTGCCGGGAGCACCTTCAGAGAAACCCGTTCTATTCTTCGGGTGATTCAGCCAGTCTCA
AAATAAATAGGTAGCTGAGTCCAGGGAGAAAGAACTGGAAACCACTGAAATTTGAGTCTTGAGGGCTGGTCTGTTCTCGGGAGCTGCTCTGTTG
AACTCTAAAAGGAATCTTGAGCCCTGGGCGAGGGAGTGGCCTGACCTGGTGGAAAGACTGCGGGCCTGCCCGTGGAGTCTATGTCTGGCCCTGC
TAGCTCAGTCAATCTCAGCAGCTCATGTCTCCTCTTAGACCCTCAGATTCCTCATCTACAGAAGGAGAAAGAAGACGAGATTGAATGATTTTTA
AGGTCTTTATCAGCTTCTAAAAGTTTGATTTGATGCTTGTAGGAGCAATTGTTCTTTCAAAGCACCATCTTCTAACCTCAGGAACGAAAGCCCA
GGGGCCCCTGGGATGTCCTGCCAAACTGGCAAGACAAAGATTGTGACTGACAAGGTGGGGGCAGGGCAGGAGAAGGGGGAAGAAGGGAGAATGG
ATCAACCCCAGGCCGGCGAAAGAGATGAATGAAGGTCCACGCCACAGTGACATGCTCTAAGTAGCGGAAATCTTCCTAACTAGAGTTCCCATGA
CTTTGGCAACATTTGAAAAAAAAAAAAAAAAAAAAAGACATCCTTAAACCCACAAAGTACTCAATTGTCCAAGCCCCATTATGGCCAACTTCCC
AAAGCTGGCTGGTCAGAGACCTTGGTGTTAATTCCTCAGCCAACTAAAAAAAGATGGTGTAAGTTCTGTTGACTTAATTGTTGGAGTGTTTCTG
AGTGGGGCCGCCTCCCTCCTCTGCCTCCTCACAGTATTATGGGCTATTCTGCATTACCTGCCCTACCTATCTTCATAATCTAGCCGCAAACAGC
TTTGGTTGACCACACCACTTCCCTATTGAAAGTTAATACTGTTTGACTCATTCTGTGTTACTAACCTGAAGCCAAAGAAAACAAGGCAGAGAAT
AACTGGCTTGTGTGGAGTTTGGAAAGCTCTTGGAGGCCCTGCTTTCAAAATCCTATACAGTAGCTTTACTATATCTCTCCGCTGCAGCTGCTGA
GAAAATGAAGGAAGCAGAAGTGGTTAAAAAAAAAAGTTGGGGGTGGGGAAGAGACTGGGAAGGCAGTGGGGCGGGGTCCCCCTCCGGCTGGGAA
AAGACAGGGTAATCTGCGGCAGTTTATGAGGGCTGTCTCCCTCACCTGAGGTCTTGCTTTCACACACCAGAGTGTTCACACAGAAGAGGGACTG
TTGAAGGAGTTAAAAGGAGAGGAAAGTTCAAAACAGTTGTTGAAAAGGCAGCTCTCCTATTGCCTGCGTAACACAGAGGAGCTGCGAGGCTGGT
CTCACCAGGCCAGGCACCCATAGGCAGAGACTCAAGGTAGACAGGCCAGGGGGAAGTTGTGTACCTTTCGAATCGGGAAAGGATTGAGGAGGGG
GAATGTTGCCGGACAGAAGGCTGTCCTGGGCGTCCATCCTTGCCCTCCATCCCTGTCTCCATCCCTGGCAAGGATGGAACCTTCCCTTCCTTCA
TTTCCTAACTCGCTCAAATGTGGTCACCAAGGTGAATAGCAGCCACTAGTGTCTTGGAATAAACACAACTTTCCCTCCCAATGTTACCTGGCAG
ACAGTGGGACCAGCATCTTCATCACCATGCTGTGCTCTGTGGGAGTCTAGGCAAACAGCCACTTGCCCATTCAGGTAACTTTAGAAGAGGTCTC
CCTCCTCACAGATGCCCTTCTCAGCAGGTGGCTGCTTCTCGCACGTGGACAACAGTGCATAGAACTGAGCCTGATGCCTGAATCAGCCTGTCAT
GACACAAACAACCGGAAGCCTTATTTGTGAGGGAGGGTCAACGTTCTGTTCAAAACAACTTGCTGCGGTTGGCTTTTGTACTAGTGAGTACTCA
GGAGCTGTGAGTGGCCAGGGTACCCTGTCCTTCTGTACCAGCCAGGACCTGCTCAGCCATCTGTCCAAGGGCTTTGCAAGGCCAGCCTAGAACC
TCAGTTGGCGTCTGTGACAGGAGAGAGCGTCAGAGTCAGATGCTGTTCCCCTGCTGTTGGGAAAGTCCCCGAGGCCCAGGGGGCAGGGCTTTTA
GAAATAGGAGGGAATTTCCTTCCCTCTTTTTAGAAGTACAGAGGAGGAGAGATCATCCTCCACACTGCTGGATCAGCATTCCAGAATAAATCTA
GTCCAGGAAAGGGGCTCACTCATTCATTCATTCACTCACTCACTCATTCATTTATTTGTCCACCATTTATGGACGCATCACCCAACCTTGGAAG
GGCCCTGCCTTTACCTCGGTCCTGTCCAAACATGCTCAGTGTCTCTGCCCATTATGTATGAGATCTGCTGCTGCGGGAGGGGAACTGAGATTCC
TGCAGGGAGGTTAGGAGGCCAGTGTGGAGAGCCTCAGTGCTGGTAACATGCTCACGCAAGCAGTGTGTGTGCGCCTCCTGGATCTTCTTCTCCT
GGCCCCCAGGTCTTGCCTGTCTACCCCAGGACTAGCCCTGCTGATGCCTGAACTCCAAAGCCATCTCAGAACCTCCAGCCACCGTTCTGTCAGT
CAGTGCTCATATCTCCATAGTGCGCCACCCGCCACCAATACTTTCTTTCTCTTCTGCAAGTCGCCTTCTCTTTAGAGTCTGTCCCTGGGCAGGA
GCTACTCTGATCAACAAATCCATGTCACTGGTGGTTACCACTCTCTGGTTACTTTTCAGTTACGCTGTAACTCGGGGTTTCCTTGAGATAACAC
CTTAACCTAGGGAGGGTTGCTAATATTTGGGATTATAGGCTGGTAGAAGAAGAAAAACCTTGAATTTGGGAGCTGTCTGCACCCTTCTTAAGAA
ACTGCCTCCACTTCCCCCACAGAGCGCTACCTCTCGTCAAGACCAAAAGCCAGCAGGTGGCTTCTCTGCCCTGAGCTCAAGTTACCTCCTTAGT
CACCAGACAGAAATGGACAGTGAGCCCTGGCGACCGGGGATGGCAGATTTAGATTTTGAGTGCTCTGCTTTTAGGAGAGGCCCGAGTCTCATGT
GAGTGGGCGAAAAACTCCACTGCTCGTGGCAATGAGCCCTGGACTCAGGGAGCAGAGTTTTGTTTGGGTTTTTGTCCCGTGAGTCTTGGGCGCA
TGGTGCAGGGCCGCTGGGGAGGGGCAGGCCTGTGTGCGTCTCTGGCTTGAAGTTGTATTGCTTCTTGATCTTGCCTTGCCGTTGGCCACCCTTT
CCTGTCCATTGACTGTTGCTTGCTACACTTGCTTGGCCCATGTTCCTGAGGGCGATCACAGAACGGTGTGTGTGTGTGAGTCGATGTATGCATG
TGTGATCGATGTATATGTATGTATGTGTATATGTGTGTATGTCAGTATGTGTGTATGTTTGTATATATGTCTCCCCATCTCTGTATATCTCCCT
GTGTGTATATGCATGTCTGTCCCTGTGTGATATATGTATATGTGTGTGTGCGTCTGTATATATGTGTCTGTCCTGGTTTGTGTCTGTATCTGTA
TGTGTGTCTGTGCATGTCTTAATATATGTGTCTGTGTGACGTATGTACATGTGTCTGTGTGTATTTATATGTGTGTATCCCCATGTGTGTTTTA
TATCTGTGTGTGTGTCCAGCTGTCTGTGTATGTCTGTGTCTGTATCTCTTTACATGCATGCCTGTGTTTGTGTGTATATGTCCATGTGTGTCTG
TATGTGTCTTTATACATGTGTGTCCATGTGTATCTATATTTTGTTTTGTTTTGTTTTTTGAGACAGAGTCTCGCTCTGTCGCTCAGGCTGGAGT
GCACTGGCATGATCTTGGCTCATTGCAACTTCTGCCTCCTGCGTTCTAGCAATTCTTCTGCCTCAGCCACTTACTTGTAACTGGGACTACAGGC
ACGTGCCACCATGCCCGGCTACTTTTTGCATTTTTTAGTAGAGATGGGGTTTCACCATACTGGCTAGGCTGCTCTCGAACTCCTGACCTCATGA
TCTGTCCGCCTCAGCCTCCCAAAGTGCTGGGATTACAGGCATGAGCCACCGCGCCCAGCTGCATCTGTATGTTTTTATGTGTATGGCTGTGTCC
GTGTGTATGTTTGTCCATGCATGTCTGTATGTGTGTATGTGTGTGCATGTGTGCCATCTGTGTTTTTGTATGTCTCTCTGTGTGTGCGTCTCAG
AGGGTACTCACTACAAAGAAACAGGAGAAGCTGGTGGACTTTGCTTATGTCAGAGTGCCACTCTCAACTCAGCAAAAGAGCCTTTGACCTATTC
TTTTTGATTTTTCACTATGAAAATAAATGAGTTTTCCTGATTGATTTAATTTTTTTTTTTTTTTTTTTTTTTGCAGACGTCTCCCTCTGTCACC
CAGTCTGGAGTGCAGTGGAGCGATCTTCCCTTACTACAGCCTCTACCTCCTGGGTTCAAGAGATTCTCCTGCCTCAGTCTCCCAAGTAGCTGGG
ACTACAGGTGCGCGCCACCATGCTTGGCTAATTTTTTTATATTTTTAGTGGAGATGGGCTTTCACAATCTTGGCCAGGCTGGTCTTGAACTCCT
GACCTCAAGTGACCCGCCTGTGTCGGCCTCCCAGAGTGCTGGGATGAGCCATCAGGCCCCGACTGATTCAACTTCTAATGAGACAATTCTCATG
AAACTCCTCAGTTATCATATTGTATTTTTTCTCAACAAGAAGTTTTTCTCTACTTACATTTCAGCTGAAAACTTTAATTACAACTTCCTTCACC
TGAAATGATTTCTGCTTTCCCACAACCTAGTGGAGGGTGATTGGACAGCACCAATCATGAAGGACCTGAGTACTCATTCATTCATTCAGCCAAC
ATTAGTGAGCCTCCTAGGCTCCAGTTGGTTTTATAGGTGCTGGAGACATGGCTGTGCAGAAAAAATACAAAATCCCTGTCTTTGTAGACTTTAC
AGTCCCATGGGTGCAGACAGACCCTGGATAACATTAACATGTGCTATGGAGAAAACCTAAGCCAAAGGGCTAGGGACTGGCTGGATGTGGGATA
GTCTGCAACTTAAAAGGGGATCTCCAGGAACTCCTCAGGGAGAAGATGACATTTCAGCAAATACTTGAAGGGTGGGAGGGATCAGCTCCACAGG
GATCTCAGGGAAGAGCTTCCTGAGCAGAGGAAACAGTAAGTGCAAAGGCCCCAGGGCAGTGACACAGGGGCAAGAGCAAGGTGGCCACTGCTGC
CGGAGCAAAGAGGGGAGCTCAACCTATCTGCCTTCTAGTTTCAAGTAGGAGGCATTTGCCTTAGAAAACAGGCTTAAGAAAATGCCACCTGACC
TCAGGTGACATCACCTGTACTTCATGTCACCTGAGAGGCCACTGTAGGACCATGGTTCGGGAAGGCCCAGAGTAGGAAAGCCCCATACACAGGT
GGACCCAGTGAGTGCTTGCTTTCCTGTTACCACCCACAAGAGTACAAAAGCTCTTTCCGACCAAAGGCTCTGCTATTCCTGCTGGTGATTCTCT
GGGGCTAAAGCATCACCCCTCTAGAAATCGCTGAGTGGGTAAAAGGGATTCCTAGTAAGTGGAGGTATCCTAGGGGACTTGCCACGATTCATCC
ACCTCTAAGAGGCTTACGGGCATCAGCTAGAGGAACAGGGGCCTGCACAGGGAGTGTGGGCGGGGTGGGTGACAGGTACTCACCTAAGACTTAT
GTGCCTACTGCCACTGATGGAAAAGGTGGGCATTCAGGGCAGATGTCTCATTGTTTTCCAACCCAGACAAGAAGCTGGCAAATGAGACCCTGGA
TGAGTGTTCCGCAGAGTGGCTTTCTTCCCAGGTGCAGCCTCTGACAACTTCCTAGCAAATTTTAAGGTGTGTTTAATGTCCCACTCCCTCTGGA
CTCACCCTTGCGATGGTAGAGGGGGAGGGGTGAAAGCAGAATGGAATGTCCCAGTCACTACCGTAGCACGAAAGCCTCTGTGAACGCTTAAGGC
AGAGAGGAGGGCACCTTTGAATACTGAAGTCCAAGGTCACTCATAACTAACTGTCCAAAGGGTCACTGATGAAACAGCTGGAGGCAGAGGGAGA
CTGAATTCTTAGGACACCTCGTGCAAAGCTGCTGACCCCCAGCACACACAAAGATAGAGCAGGCCAGGTGGGATCGCAGGCTGGGCGACCTGGT
GGGGATCAAGACAGAGCTCTGAGACCAACCTGACAGCTACACTTGGCAGTGGCTCCCGCAGCCAGGTGTCTGCCTGAAAAAAGACAAAAGGGAA
AACTTTCAAAGTCGTAACCTCATTAGAAGTTGTTCAAGAAACACCGTGCTTAGAACCAGTTGAGTAGATAATGGCAGCAGATGTAATTGTTGTG
CATGCTTTTAGAGTCCCTTGGAGTAATCCTTCTTAGTCTCTTAATTCCAGCCTTCCAGATACGGAGCCTCAGTGATGAAGAGACTTGTTCTTGT
CCACAGCAAAGGTTGGTAGAAAAGCAGATTTACAATGGAGAATTCTTAACGCCTACTCTAGCATTCACGTTTCCCTGGGACCATGTTGTTTTTC
TAGAGGGGAGGAGGATTGTGGCATCTGCATTAACACCCGGTCCTGTGGGCACTCAGAGAACTTAACTGCTGCATTTGTGGAACGGAATAAAGGT
TCCATGTAACCCTTCCCTGCCATTCCCCAAAGTCTTTCTATACATGGCACAGCAGAAATTACTTCCTACTTGAGAATCTGGGGTTCACCTTAGC
AATCAGACTGATTATCATGACAGTGACTCTGACAGAATGTGCCCAGTTATTGTAGGAGACAGAGACGGTCACACACCGGCCTATCTGCAGGGCA
CAGTTTGGGCATTTTCAGCTGCTCTGTCTCTATTGAAGAGTTAAAAAAATGAAAAGACGGCCAAATAGTTATCACCAAAAGGAGGCCGTCAGTC
ATTGCTCATGAACACAGTCTAAACACAGTTCAGGCATATTTAGTTCGCGTGTTTCTCCCCAGCACACAGCAAATCAGAACCACGCAGGAGAAGG
GAAGTAGTAATGCACATTGGAAGCTTGAGTATCAGGAGGAGCTCTCCCTCTCATGCAGATATTCCCAGCGTGCTCCCTGTATCGGCAGCCTCAG
CATCACCTGGGACCTTATTAGAACTCCAAATTCTCAGGCCCTTCACCCTAGACCAACCTACCAAACTAGAATGCCCAGGTAATTCCATTGCCCA
CTCAAGCAGGTGAAACACAGCCTCACAGCCTGCAGGTCACAGTGTGGCTGGGGCTGTCTTCTGATTCTACACACACACTGCGACCAGAAGTGAG
ATGGTTGAGGCAGAATGCAAAAGTTCAAGTGCTTTCGTGTCTCTGAGATCTAAAAGTATGAAGCATTGTGCACTTCTCCACCCTGTTCGCTTTG
AGTTCACAGAAATGTGTATTTCTAGCTGCACTGAGTGTACATCCCGAGTGTGTGTGTCGGTGGGTGTTGAATTTCTAGGCACACTGAGTGTACA
TCCCGAGTGTGTGTGTCGGTGAGTGTTGAATTTCTAGGTGCACTGAGTGTACATATCTGAGTGTGTGTGTCCGTGGGTGTTGAATTTCTAGGTG
CACTGACTGTACATTCCGAGTGTGTGTGTCGGTGGGTGTTGAGAGTGTGTGTGTTTTCAGACCTGCCCTCTTCCTGTAACTGGCCGTGTGAGAT
TGGGCAAATGACATAACCTCTCTGAACCTCACTTCCCACACATTAGTTAAAAACCAAGGCTGATTTCTGTGGCCATTCTCCAGTTCTGAAGTAC
TCCAACCTTGTCGTGTTGTCTTAGCTTAACGATATGAGAAGAAATTGGGGTZGGGCAAAGTGAGGCTACCCATAGTTACGTAGCACTGGACAAT
ATATAAGCATGCATATATAATATGAATTAGAATGTATACTGACATACTATATACCATTATATAGTTTATATTATATAGTATATATAATATACAT
GTGGTATGGTAAATAATATTGAAGAATATTTCCCAACATTAAATTAGCCAGCATATGGTTAGGTTTGCAATTAGGGAGATTTATTTTACCATCT
GCAAGGGGCTTCAGGAAGAGTCACATTCAGTACTGTCACTTTGAAATCCTAATGACCCTGCAACCTAAAGGTTAGACTGTGATACCAGGAAAAG
TAAGCTGTTCTCTACACACATTGATTTCTTTTCCATTGTCATAAAACAGTAGCAAATCAAAGGAAGAAAACCCCATATTTTTATATCCAGATTC
ATGTAGTGAGTATCCAATTGACTGGCCAACATCTTCTGAAATACAGATATCCTTTTAAAATTGACTATATAAAATTCTTATATGCTTTTAGAAG
AACCACAATTTTATACATCTATATACAGAGAGAAATATATCTTTTTGGTATTATGCTTTTGCATAGATTTGGCTCAACCGCTCATCATGACAGT
CTTCTGAGTATATACCTTCCTCATTTTTCTGCTAAGAATCTATCAACAAAACTAGTAAACCCATATCGGAAACATAGACTAATATGTTGTTGAC
TGCCAGAAAATTCTTTATTCTATCTAACCAAAATTTTCCAAGAAACGGTGGCAATTTAACCTTATCTGGTTTCTTTAATGGAAGATCATATATT
ACATTTATGTTACTTACAGCCACACATCACAATGCTGTCTTGAAAAAGGCAGCTCGGTTATCAACGAGATATGCATACCTGCTCAGTTTATTTT
GTATGGCAGAAAAGTGAAAGCTGGTCTCACTATCCGTCACAAAATATCATAACATTTAAGGATGGCCTAATGAGCAGCCAACCCCGAAAACAAT
AGGAATGCCTTTGGAATCAAGTAAAATTTTGCAAACGGCATTGACTTATTTTTATTATATACAATTTTTTCCCCTTTTTTGGATTTTTATGACT
CTGCATACATAATAAAATTCCAAGCAAGTACTTTCATTTTTTTCAAAAATGAGAAAATGGTACTAGAAGAATTTAGCACCTTTTTCAACTCTGC
TTAAGATTAAGATGTCCCAGGGTGAAATCTTGATGTGCCCACTACTTTAAACCACCGCCCTGTAAACATTCACAGTTGATTCCAAACATCCTGA
ACAACCCAAGCTGCCAGCATGCAGQTGTTCAGAGCTCCATCCTGGTTCTACCCAAGGGCTTGACCCAGGGTCGAATTCCCTGTTGCCCTCTAAA
GCCCGGTACTCAGTGAGAAAGAAGAAAAGTGAATTGGATAGTAGACATTTTCCATACTCCTCTATTTAACATTCGGGGGGCAAATGAGCCTTTT
AACTGAACACAGATACAGGAAACTGAAGACATAAAGTATTCATTTTCCCCAGCTTGAAAGTTTATATCCGAAGTTCCTATATTTCATGCAAAAT
ATTGCTATTTGCCACATGAGGTCTGATGGTTTCAGAAAGTAAGAATGAGATGTTCCTTCGATCTAGCTTGTAACTGCATCCAAACCACACCCTG
AATATGATAATGCTTCTCATTTCCCCTGTGCCTTGGTGTCACAGCTCTCCAGCACCATTGTGAGCCTGAGGGTCAAGACCCCTACCAAGGCAAT
AACGTTCCTACTGACTCCCAGGAACACACGTGGGGAGAGTGGAATGGGCTCTTCCCCAATCCTCCAGCATTAAACTCAGCCCTGAGACTCCCAG
GGCTTAGAAAACAGCTTAGAGTGAAGAATTGTTTTCTCTGAGAGTTATGCCTGAACACTTCTGCTAAAGCAGACAGTCATCAGTTAATCTCTAT
AAATGACTACAATTTTGGCAGGTATCAGCTGGTATAGAAATTGCTCACAAGTTAAAGCTCTCATTTCTAAGTATATAGTAATGAGGGGGACTAA
TTAAAATAAAAAACAAAGAAACAAACAAACAAAAAATACCTCACGTTGTGCCTGACCACACGACTGCCTTACCTGGAATGATGATTCCTGTTGG
GTGTCTCCGTGGCACAGGTCCCCACGCTTGCTGGCTAAGAGAGACACAACTGCCAAGAGCTGTTTTTCCAAGCTGTGGCTGTAGACTCTACCAC
GTCAAGTGCTGTGTATTCAGAATCACCTCGCTCACATCACTCTCCTGGGTCACCCACTGTCCTTGACACAGTGTTCTTCAAAAATCCCACTAAG
TGCCTGAGGAATGTGAAGTTTTGAAATGAGCACAAAACCTGCCGGCAGCCTTTGCCGACTGGCATTATTTCTGAGGCAGATAGAGCTCTCCACA
ATCATTGTTCCTGCCTTCTTCTTTACCCCAACTCGTAATGCCAGGCATTAGACTGTGGTAGGAGGTTAAAGGGCTGAGTTATTATGTTTTGAAT
GGTTGTTCCATTGGAATTGTTTGTTTTTCTCCAAGAAAACTGCAGATTTTTCAGTTTGATCATCAGGAAGCCAAGGATCTATGGCAGAGATAAG
AGGAGGGAGGAGCTAAGGAAGGCTTCATCCAGAGGCACTCAGTCACACCATGGCTGACCAACACAGAGCGCTCTAGAAAAAAGACATTTTCATA
CTTCTGAGGGAAAGGGATGATCGCCGTTGCTGCAAGACCTGGCCCCATGGCCTTTCATCCACAAAGCCGGTGCTTAAAATACGCAGCCAAGAAC
AGGCCTCCCACATGCATGCTCAGCCTGCGTGACTCAGGCTGTTTGCGTTCAAGTCTCCTGTTGTGGGCTGGAGCGCTCAATCAGAGTCCAGATT
TCCCTTGGTGACACATGACAAGAGGGGCCATTGCATTTGAACTACATAAATATTATGCTTAAGTGTTTTTCCTCATTTTGGAGCCAAACAGGTC
TCGTTAATGATAAAATCACTGCAATATAGTGGGCCCTTTTTTTCTGGTCTGGAATTTGAGACAATGTCAACTGTGCCAAGGGGCCTGGCAGCAG
GAAGAAATGGCCAGCCATGGAGATTTGCAGCCATCATGCAAGTTATACCATTTAATTTTATAATTGTAGCCATTGGGAGCTGGACTACAGTGCA
CTTCTCGTGGTCTGCTCTTACCCCAGGCTGCGGGAGTGGAATCAAGGGGGCCTCTTTCTAGTCTAGGATTGGGTCAGGGCATGTGAAATAGCAC
TTTATGGGGGCAGCGGGCAGGCTCTTTCTCAGTCTGGCTTTGGGGACATTTGGCAATAGGGAACAGAGGTTGTGCAATCGATCAAGGACTCTTT
ATTGGAACCATAAGGCTGAGGAAGCAGCAGGAGGCCATTGTCTTTAAAAGAGAAATGAATGGCCCAGGCCTCCTGTGGCTACCTCCCTACCTTG
GAGCCTCAGCTTTGCACCACAGCACTGGCTCTGGTTTAAGGACATTTTGAGAAAACCGTAGGCCCCAGTCCCCCATGAGTGGCCTCTGCTTCCT
GCTTCCTGGTGACGGTTGTTTTGGTGAAGAGCATGATGAGGACACTTGCTATCTAAATAGACACTGAGGGGCCCACCATCCCACCCTATATAGA
CACTGAGGGGCCCATCATCCCATCCCACCAGTCCTGAGCCCCCTGGGGAAGCTCACCAGATAGGCTGTAGCTACCTCTGGGCTCCAGTGGGCCT
ATTCTGCTGGGAATATTGGACAAGACACAGGCGAGGCCTTCCTGTCAACTCCTCCTTAGGGGTACATGGGGGAGTCGGCTTCAGCGAAGAAGGA
ATGCTTGTGAAAAGCCTGGGACGACAGGGAGAGGGAGAGGGGCTGAGGGGCTGAGGATGCCCTGCGTGTACCTGGAAATGGGACCTCCCCAGAC
GCTGTGGGTTTGCAGCCACGCCTCATTTCCTCTTGGAAGGGGCCCTACCACTAAGCTGCGGCGCCCACTTTGCCGCCTCTAAAACGTCCCAGTG
GCTGCGTTTGCAGTAAGGAACAGCCGTTCTCATGCCCCTCGGAGGCCACCTTTCCTGGCTGCCACAGCATAGCCCCTGGCATGTGGCCATGTGC
CAACAATTAATGCGCCTCTTTCCCTCCCGGCATCACCACCACCGCCAAGTTCTGTATCTCAGTGAGTTGTGCCCATTGCAAGTGGGTGTAGAGC
GTTGCGGAGGGTGCCAAGGCCCAGACTGGGGGAGCACTCTGTGGCCGAGGCGGTGAAAGGGGGCCCATTCTGCTGAGAGGACAGTGGCCCCAAA
TTCAGCTGGCATATCTCTAGCGAGTCTATGTTGGGGTGAGGGGAGAGAGAGGGGAAAGACAAGAAAAGCCCCACTTTTAGGAAATCCACAATAC
TTTTTCTGATCTCTTCCCTCCCTCCTTCCCTCCCCCCATCCCCTCCCCTCCCTGCTCCCCACAATAGGACATCGGCAGAAACTAGATGATCAGA
CCAAGCCCGGCAGCTTGTCCTTTTCCGAGCGGCTCAGGGAACTGGAGCAGCTGCGGCGCACAGCCATGAGGGTCAGCCCACACCACCCAGCCCC
CACGCCCAACCCTCGTGCCTCCCTCAACCACTCCACTGCCTTTAACCCTCAGCCTCAGAGTCAGATGCAGCGTAAGTACCAGATGGAGCCCACT
GCCCGCCTCTCCTGCACCTGGGCCACCACCCACAACTGGCCCCCATGTGCACACACCTTCCCAGACCAACTGGGGTTTCCCCTTGATGCTCAGA
GAAAGQCCTCGAACCAACAGCACCACCTGGGAGCTGTTGGAAATGCAGAGTCTTGGGCCCCACCCACCCCAGACCCACCGACTCAGATCTGCAT
TGTAACCAGATCCCCACAAGGAAAAGCACTGCTACAGAGGATACGAGGAGCTCTGGGTATGGATATCCATATTGGATAGATAGCTCTCTTGAAT
TTTTTAAGAGTTAGCTTTGCCTATGCTATTTTTTTACCTACCCATTAGCCTTGAAGACACACACCCACACACACACACACACACACGTCAAGCA
AAGTGAAGATGGGATGTTTCAAGTTCTCGCTATTGCCAGATTATTTGTGGGTTTTGTATCTAGTGTTTTTTTATTATTAAGAAATAGTTGAAAT
GTATGGATGTCATCACATCAAGGGTGTTATTTGCAAATCAATAGAGAATGCAGGTCCCCCCAGCCCATGGGCCTAGCTGGCAATTACTAAAGCG
CTGTAACATGCAATAATTGCCTAAGGCCCACTGTGCCAAATTAGATAATACAACAAGTTCATTTACACTGTAGACCAGTGACGTCAATGACTGT
TTGCTCTGTGATACCGTTTCAAAAATCCAAAATGCAGACTTTTCTCTCTGCCATGCAGGATGCAGCTGTGTGTGATATGGTTTACAGTAATATT
TCTTTCTCAAAGTAGCAGGCTTGTTAAGGAAAAGATAAGCAACACATCTGGGGAAAAGGGCAGGTGGCCAGCAATCGATGTGGTAGCTCTTTGC
CCCTCTCGGACAGCAGGAATTAGCTTCCCCAGGCATTTTCTTATGTGAGTTGTATTGTGGGATGTACAAATATCATCGVGTTCCTTTGGGTTTC
CAGGCCAGTAGCTCTCTATTTTGGGTTCAACATGGGTTCTCAGGCCGCGCGCGGTGGCTCACACGTGTAATCCCAGCACTTTGGGAGGCCAAGG
GCGGGCGGATCACCACGTCAGGAGATGGAGACCATCCTGGCTAACATGGTGAAACCCCAACTCTACTAAAAATACAAAAAATTAGGCAGGCATG
GTGGCGGGTGCCTGTAGTCCCAGCTACTCAGGAGGCTGAGGCAGGAGAATGGTGTGAACCCGGGAGGTTGGAGGTTGCAGTAAGCCGAGATTGC
ACCACTGCACTCCAGCCTGGGCAACAGAGCGAGACTCCATCTCAAAAAACAAACAAACAAACAAACAAAAACATGGGTTCTCAAAAGGCATGCC
CACTGTCTCCCATGGAGCTTGACAGCCCATGCCATTAGCTCTCACTGTTAGGTTTCTCGGGAAGGTTCTTCTACTTCATTGGAAAATTTCCAAA
TAATCTTTCCAGAAGATACTATGCACACAGCTAAGTGGCCTGTCTGTGGAGTAACCCTTTTGTAAACAAACAGAAACCTAAAAGCTTGATGTTT
TGGGGGGCTGCCTGTCATCTATAGGTTCATTTAGGTGTATTTAGGAAGACGATCCATGAAACCACTGGTTTCCTGTTACATAATAATCATTAAT
AATGATTTAAAATGTGTACATTGATTTTTTTTAAATTCCAAAATACAAGCGTATATGGTAAATTAAGTCAAATGGTATGTTCAGTGAGCGAGAT
GGGCTTGGGGCAAAACAATACTTTGCTTCCAAAGAGGATACAACTCTCAAGGAGATTCTTTCATCTTGCCTTTAAGGTCATTTAAACTAATTCA
CATAATCTTCAGAAAACTAATTCACATCATCTATTCATGTCTAAAATCAAAAGGAAGACTGTTTTCTTAGTCTCTCGTTGCCTAACTGGCCATT
TATACTACTAGGTTGATTAAGGGATTTGCCTTTTTCTGCTGATATGGGAACAAAAAGTCTTAAGCATTTTTAAAGCCAATGGAAAATTCAGCCA
CATGGGGGAAAATTGATATTGTCACCATTGAGTTGCTCTGTTTCTTGGTGAAGAGTGAATCTAATCTGATTTCCTTCTTCATCAGATATGCCTC
TTTAACAACAAAAACAAAAAAATGGGGGGAAGTTCCACATTGAGTTCTCATCCTCTGAAGCTCCTGGGCAAAAGCATTATTGTGGACCGTGTGA
TGGGCATTCGTCAGCTCTTTGGAGACCTATATGCTTAGTAGAGTATCACGTTTAACACTAAGCATCGCCCCTTTTTTGTTCCTCTTGAAAACTT
TTACTTCTCCAACATTTAAGAAATCATAACTCGCTTTTAGAGTTCCTACGGGCTTGGTTGCTACTCTAAAGAATGTGTGAACTATATTGCTGCT
GTTCTTTCTTTCCAAAATATGAATTTGATATTCAGTACTTCACTTGTAAAATTTTAGGAACAACTTTTAAAAAATAAGTATCCACAATATTTTC
ATGTGAAACAACTTTTCCTTTTCCTTTTGTTCTTTCTTTCCTATAAGAAAACGCTGTTTTACGCTAATCATGTTTTGAGTAGACCCTTGGGCTT
TTTCTCTTGAAATTGGAAAGTTAACAGGTATAATATGCAAGTCTTGTTCAGCCAAGACCTTCAGTTTGTTCTTGACAGTAGGAAAATGCTTATT
TTCCAGGCTCAGCCCTGAAATTTCTATGTTATATTTACGAAGATACTTTAAGAAATACAGCATGTGTTTCTGGCTTTCTTTGAAATGGCAGTGG
TAGTTTTCAGAAAAATATCCAATTTGTCAGAGTAGTTCCACAGTGGTGCTGTCCAGATGCATGAATGTTTTTCAGCTTTACAGAAATGTCAGTA
ACCGGGGGTTTGGCGAGCTTGCCGGGATTTATACAAAGGCTCAGGAACCCTACCTTGCTAGGACTTTGTCAGAAGGGTTATGCCAGCTCCTCTG
ACTCTGTCAGCAAGAAAATACTGATATAGAATAGCACCAATACTTACCTAGAATCACATGTGCTGGAGGGTCACTCAAAATAGACATCTCAAAT
TATCTGAAGCACGGTACAGATTGAGAGGGGGCAAGTTGTCATGCCACTAAACCACTGTTTATTTGTTGGGGTCTTAACTATTAAAGCGCTGAGG
TTTATTTCAACTTCCTATTTTACCAGGTCTTAAACTGGTAAAGCCATTTTGTCATGAAATAGAATAGAAAGCCATTTTGTCATGAGTAGACCCT
TCGGCTACACGTTTCTCCCAGTCAGGACACCATCATTAACTTCTACGATCGAGTCTTAGAATTCTTTTTTAATTTATTTCAAAAGATAGAACAG
GGCAGATTCTAGAGCTAGTGGTACCGATGCCTGTTCAGTCACAACTCCCACCTAGAGATGGGTTTGGGCAACCCGAGTGAGTGATTTTAAAATT
TCACCCCTGATTGACCAAATATGATGTATCACATTTAACGTGTCGTAATTTCCTCCTTTCTGGGAATATGAGGATGGAGTAATAGATGCTGGTA
CAGAAAACTGAAACCTAGATAAACTAGGAAGCCCATTTTGTTGTTCTTCTGCATGCTGGTTGTCATAAGCATGTAGGAAGAAGATTTAACTCTT
TAACTGGACCAGTTGTGAGCTATTTTCACGTGGGTCAGACATTTGTACATATTATTCCATTCCCTCAACAATCCTGTGGTATCATTTATAGACA
GAGAAATCGAAATTCAGAGGTTAAAAATTCCCCAGTATCTCAAAGATTGTAAGTGATGAACTTGGACGCAAACCCAGACCTTCCTTGCTACAAA
GCCAGTGCAACTGGGATGGCCGTTCCCTGGGCGTGCACTCTTGGGTCAAGGATCGATTTACTACAGCGCTGACCAGTGTCGACAACAATCCCAA
TCACCAAAGGAGGGATGGAAAAAGTGTTGGCCTATCCACTCACTTGGCATTCTTGTATTGAACATGTTCTATTATAATTAGATGTTGATAATAA
GAAGAATTTTGGGGCCAGGCTGTGTGGTGGGCACCTTAATAAACTGTCTCCTTTGGTACATGTCCATTTGTTTATAAAGTTTGGGAGAAAAGAA
AACTCAGAATAATGAATCTCTAAATAAGAGTGAAAGAGGGGGTATATGAGCAGGGAGAAGGCTTACCAGCCACTCTGTTTACTTTATTGGGGCC
ATTCCCTTCCTACTTTTGAAGTTCATTGGGACCAAGTGCTTATCATCCAAACCCAGTCAACATCTAATCCACTTTTATGAGTATGCGTGCCTCA
CTTTGAGTGTTCCATATGGCCAAGAGCGGTCCCTGACCTCTCAAATCATCTCTTGATTTTTAGTATTCCAAGTAACTTCTGTCTTGCTAATTAT
TTTCCCCCAAAATTAAAGTTACTTAGCAAGGACTATATTCTGGTGCTGCTGTCTGCAAAACTCTGAGATACTGAAATGACCCTGTTTCTCAGTT
GTATAGAGTTATTTAACTCCGTCTGATTCTCTCTTTATCATCTGGATAGACAAAAGGAAGAAAACCTTTCCACCTTCCTGTAGGAGAAGGAGAT
AAATCCTCTTTCCAGCCTTGGAGGTAAAATATCCAACTGCATTGCATTGCTTTCCTTTCTCTCTCCCTCTCCTTCCCTCTCTTCCTTACTCTTC
TAGCAATTTGAAGTTTCAAAACAAAGGGAGTAGGGACTATTTGATTACCTTGAAAGTGGAGGCTACAATCAAACACCAGAAATCCTCATATGTG
AATCTATAAATGGTCATTTAATGGTTACACAAAGTAACATATTCAGAATGTGAGAAATTAATTTATATAATAGGCTATAAACATGACAGGTGAA
TGATTTCATTTAAAAATAGCTTTTAACAAGGTGTAGTAGGGTATGAGAGCCTTTCCAGGTCTATAGCATTATAATCTTACCCCGACAACAAGCC
TCAATCAGGTGTTCACAGGAATAAATAAAAATAATTAAAGGAAAGAAGCGACAGAGAGAGAAGAAATGAAAACCAGTGGGATGACTGCACAGAT
TCACTGCAGGGCTTGATCTTAGTGATGAAATATTTCTTCTGTAGACTGCATATGTTCTGAACTTTCCCCTTATGCTTCTAAATATGTAGGCCAG
CACACCCATGCACTAAAAGGTTTCATTTGGAAGATTAATAGTTTCAGCAAAGTTGTAGTTGGAGCATGTATTTGATATTGATGGCTACATATCA
GAAGGCTTGAGCTCGGAATCTAATAGCCAAGAAAGATAGCAGATCATAGTGGCTAACAAAGAATGAGGGCTCAACAGGCACACTACCTTAGTTT
CTACCACATTCACTATAGGACTTTTGGCAACTTACTTAACCTTTTAAAGCCTTAGTTTTCTTATCTGCAAAAGACGACTAATAATTATACTAGC
AGATCCTAGGACCACTGTGAAACATAAAGCAGTGGACATTAGAGCCAGCCCATGGTTAACACTCAAAAACTGTTAGCTGTTATGATCAACATCA
ATAATATCAAGTTGTCCTAGTCTTATATATTATAGACTGGGTAATATATAAAGAAAAGTTTATTCGGCTCATGGCTATGGAGGCTGGGAAGTCC
AAGAGCAAGGTGCCATCATCTGCTGAGAGTCATCCCATGGCAGATAGGTGGAAGGGCAGAGAGCATCTGTCAGAGAGCTCACTTTTATGACAAA
GCCACCTCCATGATAACTAACTTACTCCCTCAATGGTAGCATGAATTCATTTGTGAGGATGGAGCCCTTATGACTCAATCACCTCCCAAACGCC
CCACCTCTCAACATTGCCATGATGGCAACCAGGTTTCCAACATATGAACTTTTGAGAAGACACATTCAAACCATAGCATTCCACCTCTGACCTC
CCCAAATTCATGTGATTCTCATTTTAAAAATACATTTATTCCATCCCAGTGGTCCCCAAAGTCTTAACTCTTTCCAGCATTAACTCAAAAGTCG
AAAGTCCAAAGTCTTGTCTAAACCAGATATGGGTGAGATGCAAGCAAACATTTAAAGCAAATTCCATTAGCTTTGTGAAATCAAAAACAAGTCT
ACTATAATACTTCCAAAATATAATGGTGGGACAGACACAAGGTAGAAATTCCTACTCCAAAAGAGAAAAACAGGCAAGAAAATAGGGGTAACTG
GTCCCATATAAGTTCAAAATCCAACAGGGTAAACAATATTAAGTCTTAAAACTCGAGAATAATCTCCTTTGACTCCATGTTCTGCCTTCTCGAT
ACAATGGGGCTAAGACTAGGCCCCCAAGGCCTTGGGCAGCCCTGCCCCTACAGTTTTGCTGGGCTCACCCCAAACTTCACCTCTCTCACGTTGA
AGTCTTGTGTCTGCAGCATTACCAGGCTAGAGTTGCACACTGGTGGCTCTGCAGTTCTGGGTTCTCAGGGATAGCCCCAATTCTGTGTCTCCAT
TAGGCACAGCCTTAGTGGGCACTCTTTGTGGCAGCTTCAACCTCACATTTCTGTTCAACATTGCCCTGCTAGGTACTGTCTGCAGTGGTATTCC
CCTGTGGTAGGTCTTGGCCTGAGCTTCTAGGCAGTCTGTGGCATCCTTCGTAATCTAGGTGGAGGAAGCCATGCTTCCACAGCATGTATTCTGC
ATGCCTACGGAATTAGCACCACATAGAGACCATCAAGGCATACTGCTTGTGCCCTTCAGAATGGCAGCCTAGCTATACCTAGGCCCACTGAAGC
CACAGCTGGGGTGCCCAAGGTGTGCTGCACCAGGATGCAGGAAGCAGAGACTAGAGGCAGCCCTGGGCAGTGAGCCCATGAAGGGTCCCCCAGG
CCTGTCCCCTAAAATCATTCTACCCTCCTAGAGCTCTGGGCCTATGATGGGAGGAGCAACTTGAAGATCTCTGAAATGCTTTTCGGGGTCTTTC
TCCCATTGTTTTGATGAACAGAATCTGGCTTCCTCGCATTCATACTAATCTCTTTGGCAAATGGTTGCTTGGCCACACCCTTAGTACTCTCTTC
CAAATACACTTTCACTCTTTACATGGCCAGGCTGCGAATTTTCCAAATCTTTCCACTCTGCTTCTCTTTTAATTCCAAATTTCATCTTTAAGTC
ATTCTTTTGCTATCACATCTCACTATATGTAGTTAAAAGTAGCCACACAGCAGCCTCAATGCTTTGCTGCTTAAGTATTTCTTCCACAAATATC
CTAGTTCATCCCTCTTAAGTTCTGCATTCCATTAAGTCCTAGGACACAGACACAATTTCACCCAGCTCTTTGCAACCTTATATTTTATAAATAT
GGCCTTTACTCCAGTTTCCAATACCTTGTTCCTCATTTCTATCTGCAGCCTCATCAGAATGGCCTTTACCAGCATTCTACCAGCACTCTGGTCA
CATAACTTAAGTAATCACTTAAGTAATCTCTACGAAGATTTAGACTTCCCCTAGTTCTAAGGTCTTCTTTCAGAGCCCTCACCAGGAATTGCCC
CTAATGGCAGAACCGTTCTTGGCAATGCAGGCTTTTTTCTAGCCTGCTCCTCCAAACTTGTTCAGCCTTCACGGTTATACAGTTCCAAAGCCAC
TTACACACTTTCAGGTATTTGTTGTAGTAACAACCCCACATCTTGGTACCAACTTTCTGTCTTAGTTCTTTTGTTAAATGCCTGAGACTGGGTA
ATTTATAAAGAAAATAAATTTAATTCACAGCTGGGCACACTCGCTCATGCCTGTAATCCCACCACTTTGGGAGGCCAAGGCAGCCACATCACTT
GAGGTCGGGAGTTCGAGAGCAGGCTGGCCAACATGGTGAAACCCCCATCTCTACTAAATACAAGAAAAAAAAAAATTAGCTGGGCATGGTGGCA
CACACCTGAGTCCTAGTTACCCGGGACCCTGAGGCAGGAGAATCGCTTGAGCCGGGGAGGTGGAGGTTGCAGTGAGCTGAGATCATGTTACTGC
ACTCCAGCCTGGGTGACAGAGTGACATTCTGTCTCAAAAAAAAAAAAAAAAAAAAAGAAAGAAAGAAAACAAAAGAAATTTATTTCATATGGTT
CTGGAGGCAGGGAAGTCCAAGAGCATGGTGCTGGCATCTGGTGGGGTCCTTGCTGCATTAACCCAAGGCAGAAAGCAAGGGGCACAAGAAAGCA
TGTGCAAGAGAGCTTGCTTTTATAACAAAGGCACTTCACCAATAACCCACTCACATGATTATGGCATTAATCCATTTATGAGGGCAGAATCCTC
ATGACCCGTTTGCTCCCTGCAAAGGCCTCATCAACACCACCACAGTGGGATCTAAGTTTCCAACATGTGAACTTTTGAGGGACATAATCAAACC
ACAGCACAAGTATATTAGTTTCCTGTGCCTCTTTTAACAAATAACCACAAACTTCGTCGCTTAAAACAACAGAAATGTATTCTCTCTTAGTTCT
GGAGGCCAGAAGTCCAAAATCAAAGTGTTGGCAAGACCATACATTCTCTGAAGCCTCTAGGGGAGGATACTTCCTTATCTCTTCCAGCCACTGG
TGGCATTCCTTGGCTTGTGACTGTATCAGACTGTATCATTCCAACTTCTTCTTCCCTCTTTATATGGCCTCCTTTGTGTCATCTCTTTTGTCTA
ACGATACATGTTACTATATTTAGACCCACCCAGGTAATTCATCATGAACTCAAAATCCTTCACTTAATTATAGCTTCAAAGGCCCTTTTCCAAA
TAAGCTCACATTCACAGTTCTGGGGGTGAGGAAATGGTTCTGGAGCCACCGTTCAAACTGCTGTATCTTTGTACAAAAGACAGGCGACATCAGA
CCATTGACCAGGCACAGCAGGTGAGTCCAGAGATACTCTCCCAAGTCCATCCCCCAGAACAAACATGGTAGACACGTAAATGTGAAACTGAACA
TCTCATGCACAAATGCCCCAATCGTCTTACTATAACCTCAGTCCATCATTTGTTTGAACGCAACATCATAGTATTTTTATTTCCCAGCCAGAAA
AATGTATGATTTTTTTTAGATGAGATCAGATATTTCCTTAATATTTTCTAGAGCACCTATACCGATGGAATCAGAGGTCCTGACAGTGATAGAG
AAAGAGCTCTCTTCCCTGTGTCCCCGGAGCACTCCCAGATGGGGAAACAGGAGGCCCACGTGCCTCTTGTCCTAGCCTCCAAAAGCTCATGACA
GCTTAGATCGTAGGAACAGAAAAATGGGAACAAAAGATGACATGATCACCAAAGCCAGACAGTTGGGATGGACTCAAGAATTTGACCTCCCAAA
GCCAGGCATGTCACTGTTGTTTTGAAGCAAGCTGCCTTTTAAAAAAGTGACTTGATTGCCAGGCATTGTTGACTACACACCCATTTCTAAAGGA
TGGTGATTTACAAATCGCCTGAAGATAGTCTTTCAAATTTGGACATTTAAAAAAGAAACTTTTACTGTAGTCATGAAGTAGTATCAAAGTTTAC
CACAAGTTTGTATTGAGAGAAGAACAAACAATATATGCTAATATGAAAAACAGCTCTACTTAGAAAGCTACTGCTTGGGTTTTCTTATTAGGCA
TAGTTCTCCAGACTGAGTTGGTTTTACTCATCTACATGATTTTTCCTTGCCTTATGGAACAGAAATTCACGCCCACTCGAATTCAGTTATTTTA
GGGCTCTTTAAAATCCAGTATTTGTGATTTAAATCATGCGGAGGGACTTTCATTACCTGTGTCTTTGCTTATTTCTCTCTGGCCCTCAGAACAC
CCCACCCTGACCTTTAGGGGAAATTGACAGAGGCAGAGGGTTTCACCTGCCTCAATTGTCACCAGCCCTGTTACATTCTTCCTTCCAAGCCTTA
GCCTCACAGGGACCTTCTCATTATTGAACAATTGCCTTCAAAGCAGTAGAATAGCCCAATTGTTATGGAGATTAAAGATACCGATTGCAAAACT
CCTGTAAATAAAATCTTCACTGACAAACCCAGTTTCTTTTCATAGGCTTTTCTTCTGTAATCTCTTTCTGGCAGAACATCTCATGTTTTCATGT
TAGAGATTCAGTTACCAACCACAGTAAATAAAGCAAAATAATAATAGAAAAATAGTATAGAACTCACCCTAAAAACAAACATTGGCCAACCATG
TTTATTTTTTGTCTCTCTTTGCACTCCTGAGAATTGATAGGGGAAGAATGTACCACCTCTAATTCAGGTGATTTCTGATTAGCAAGCTATGGAA
AGTCTTCAGGTTGAGTTTTAGCCAGTTCACGCTCCCCTAAATCGCATGGAATAGACTATTTTCTGTTTTAAGAAAAAATAGAACAATGGCACTA
AATGCTTGACTGAATGTTTGACTAAATGTTGACTGAATCATGGATAGGAAAGATTGGCCAGAAAAGACAGCCACTGCCTCCAGACACAGGATGC
CACAATCCTGGGCACCATCATTATTCCATACAACCTTAGGGTCATTTTTAGGGTTTAGAACTTTCTCAATAGGGTTTCAAGATTTTGAAAAGTG
TCTTCCAATTCTGATCTCCGTAGATCCTGTTATGGGAATTAACCTTTTTGGAAGGGGATTCTTGTTCTTAAAGATGAAATTCCCTACTTTCTTT
CCTGGAGGGAATCAGTATGGGCACAGGGAAGAGGAGATGGCGATTCTCACCTGTGTGTCTCATGTCACCTAACACCTATGGGGTGGCATGAAAC
TTGAGCTTTAAAACACACCAGGGGCCAGGCACAGTGQCTCATGCTGGTAATCCCAGCACTTTGQCAGACCCAGGTGGGTCGATCACCTGAGGTC
AGGAGTTCGAGACCAGCCTGCCAACATGGCAAAACCCCGTCTCTACTAAAAATACAAAAATCACCTGGGTGTGGTGGCGGGCACCTGTAATCCC
AGCTACTTGGGAGGCTGAGGCAGGAGAATCCCTTGAACCTGGGAGGCAGAGCTTCCAGTGAGCCGAGATCAGGCCATTCTACTCTAGCCTGGGT
GACAAGAGTGAAACTCCATCTGAAAAAAAAAAAAAAAAAAAAAAAAAAAAAACCCACCAGGAATGGAAATGGTGCTTAATTCCTCTACCGTCTT
GGTTCTTCCACACAATCTGAACTGGGTCACTGAATCTTAGCCTCCGTAAGTCAACATCATCAGTTCCGTTTAATAAGACTTGGGCCACTATAAT
GTCCAGCTCAATCCTCCTGGAAGTATGGACGACACTGCAGGATCCTTCAGTTGATGTCATAAAAGCAAAGCATCCCTAGGGAAGATCTGTGAGT
CAAGCCTCAAGGCCCTCGCTGAGTCAGCGCTAACTTGAGTATCACGAGCTCACCCGTCTGGGACGGTTAAGTCTCCCAGCCCTGTAAATTGCCA
GTTCCTGCTATGGATGATGTCCTCTTTACCTTACGGCTTTATGTGTTCAACCTCAGTCATTCTAAAGCCAGATCCAACACCCAGAGAGCTTATG
CTGTGCCAGTTCCTCTTTATATCACTGGAAGGACTACATATGTCCAGTTGCTTTGGGATTTTTTTAAAGAACATCTCAAGATAAAAAGGGAGGA
AGAAATAATGTGACTAGGGATTGCACTTCCTGGAGTTGCAGGTCACATGAGGGACACGAACATGGTGGTTTGATTAAGTCTCGCGGGATTATAA
CACTGTTGTGGCCACTCTCACTCCATTAGTGCATCTCTCCGCACATATCAGATAGCAAATTATATAATGATAAAGTTATTAAAATATCTGTAAA
AGTCAGCACAAGCTCAACGCCTCGACTTGGCCTTTCTGCCCACTAAGTGGTTTATTTAGCTCTCTCATCTCAAGGCCGQTTAATCTTCATCTAT
ACTTTGTTGTAAATCACCCGCTATGTGGAACAGCCATAACTGCCTGAGTTTTAAGATGCGCTTCTGTTGGTCGTCCAACAAGGCTGGTATCTTA
AAGATGAATGCAGAAGCTTTTGAAGCATGAGACTCACCCACTTCCCCGTTTTAAAATGATAGCCTTTGTTTGTGATTGTGTCTATACCTGGGTT
ATCACAGAGCAGTTATGACACCAACATTATGACAAGGACATTGCCTGGTTCACATGTTTACTGAAGGGAGTAATAAAATAATGATAGCTCCTAT
TTATAGAACAGACCAATGTTGCTGGTTCATTGAATCCCCACCACCAGTCACCTGTAGGTGCATGACATCGTTAGGGGGAAGCATTGCCCCGAAG
GTCCTGCCTGTGGTGTCTGGTTTGTTTTCACGTGACCCACCACTGTTTCCTTTACCACCAAGTCCCAGTCCCTCATCTATCATGTAAGTGATTA
ACCAGGTTGTCTTCAACACTAACATTCTGTCATTCTGGATACTGCAGTTCCCGTGAATTAATGCTCACCCCTAACTAACATTCTCAGGAAAACC
AGTAGGGTTGCCCCAGAGCCCCTCACTCTGCTGCTTATTTGGAAGGAGCCTTTATAGTTCTGCTGTGTGAGAGGCTCTGAGCCACCATATGTTC
CTAAATGAGACAAGCCGTTGCCCAGACATGGATCTGTTTAATTTAATATGATTTACCACAAATTACGAAGCATGACTTCAAAATTGAATTTGTG
CAAAATTACTGATGGCCAATTCAGTGGAAGGGAAGGCAAATCTTGTTATTCCTATTCCAGAATCTCTTCCTGATTTCCTCATTTGATTCTTTCA
CGCCTTGGGGTAGGGGAGGGGAATGCATTCGCGGCTCTATAAAGAATTGTCCTTATTTTCGAACTTTCTCCCTGGTCACACATGGCTCCCTCAG
AGGAAGACACAGCACCCTGGAGATGTTAAGGCAGAAGTCAGTTCTTCTGTCCATCCCTCTCCCCAGCCAGGATAGAGCTATCTTTTCCATCTCA
TCCTCAGAAGAGACTCAGAACAAAGATCACAGCCCTCAGAATGCACGTTATGAGGAAGGCAGAATGTGGGTCTGTAATTCCTCCGTGTCCCTTC
TCCCCCTCTGCAAACCGTCGTAACAATAATAGTTCCTAACACATGGGACAATTGTGAGGATTAAATGAGTTAGCCTGCAGAAATCACTTGATGC
ACAGCACATGGGAAGCATTGTGTCTATTTATTAATCCTTCACAAAGTCTTTGAGATATATTTTTATCAAATATTTAGCATGGATCCCGGTACAC
TTTCAATACTTAATAAATGGTCAATGTTATTCTTTTTCACTATTATTGCTATTTATGTGCTCTTATTGGCGGCATTGTTGTATCACCAGTTAGT
TGTACCCATCGGTCCTCTCCAAGTCACACAGCTACTCTGCGATAATTCCCCCGGTGTCTCCTTATTCCTCGGGAGCCCCAGAGTGGATAAAGGT
ATTGGTGGTTTTAGTGGTATTTTCATTTGAGTTTTGTTTCCTTGGATAAACCCAAACAGGACACCTTCCCATAACCACTGTTCCACAAGACCAA
ATGGTAAACTGTTAAAGTTGAAAGGACCTTAAACTTCTTTGCTCAAAGCCCTATATTTTACTCATGAGACACTCGAGGCCCAGGGAGTTTCAGT
GATTTTCCGAACTAGAGGCAGACCTAACATCCAGGTTTTCTGGCTCCTCGCCCAGTGCTCTTCCCATTCGCTGGTTCGGGTCTGCCTCAGAGGT
AGCCTCAGAACAGTCTCTAGGGTTGGTGTGGATCATTCACTTGGGCACTGAACCATGCTGTACGTCACACTGCCTTTTTCTGGAAATACACCAT
CTCAGGGAGATGCTAGGTCCCATGGTTGGGTTCAGTATCATGTGCTGCTTCTGAGTCTCCATTTTTAAAAGGTTGAATTCGTGCTAAGGAGGAG
TGATGCTTTTTTTTTTTTGAGACGGTGTTTCACTCTTGTCACCCAGGCTCACTGCAACCTCTACCTCCCGGGTTCAGGGGATTCTCCTGCCTCA
GCCTCCCCAGTAGCCGGGATTACAGACTCCGGATTACAGAGTCTGCCACCACCCCCAGCTAATATATATATTTGTATTTTTAGTACAGATGGGG
TTTCAGCATGTTGGCCAGGCTCGTCTCGAACTCCTGACCACAGGTGATCCGCCCGCCTCGGCCTCCCAAAGTGCTGAGATTACAGGCATGAGCC
ACCATGCCTGGCTGGACCAGGGATTCTTAAAGCATGCAACGAAGGGAAGTCCTCCTCAAAGCCACACCTACCCTCCTCAGCCCCACTCAGCAAG
GAAGCGAAATCCTCCTCAAAGCCACGCCCATTCTACTCAGCCCCACCCAGCAAGGAACGGATATCCTCCTCAAACCCATGCCCACCCTCCGCAG
CCCCAGCCGGCACCCCAGCCCTGCTCCCTGTTGGGGACAACCAGCTACAGGAGACCGACAGGTCACTTCCTGAGCACAAAGCCCACGTGGGTAG
CAGGCAAGGGTGAACCCTGACCTCTCCCATTACTTGCATGAATTGGATGCATTCAACAAATGTGAATCGTCAGCCTCTTTGGTGTTTTTAATGA
AGCAGCTACAAAAGGATTTCTTTTAACCAAGTTTCACCGTCCTGTGTGATTTTGAGTAAATACCATCCTGTGCCGATTCCTCCCACAAGCCCCG
CCCTTTAAGCCTGCTTCGGGCCTGTGGCTTGGGCTGGCGGGCCAGAAGCAGGATGGCAAACAGGTGTGCGAGTGTGACAGGGCAGAGGCTGGAG
CCACAGCTGTGGGAATACGAGGCTTTGGCAGGCCGAGGCCAGGAGGGGCGGGAGGCTGGTGCTGACTGATGGTCCTGTCATCCCGGTTCAGGAT
GCCACTGCCAAACTTCATGCCAATTGCTCCCAACAGAGTACACCGCTATGAGAGGGAGAGAGCTGGGACCAGATGAAGGCATCCCTGTGACGGC
CACGTCCTTGTCCATGAATTCAGGGTCAGGGTGACGAATCGCAGGGCCAAAAGCTACCCTGGAATCTCACAGGAATTTGCAGAGACCAAATGCC
CTTCTTTCAGAGGCACCAGAGTGGGGGCTCCCGCATTGACGGGTGGCTTCTGGGCCCCCTCCCTTCACTAAGACTTGGTGACTTGGTAATCCTC
CCTCCTCAATGAAACTTATCTAAGAATTGATTAGGAACACGGACTGGAGCATGGCAGTAACTAGCAGTGCAGACCTGTCATCTCCATCTTCCCC
TCCCATAGCGTGTGGCTCTTGCCATGTCATGACCACACTCCCGGGGGCTCGAGCTCTCCGCTTGCCTCAGCAGTCTGAGAACCACAAGTTTCTA
AAGAACACGTATGAGGAGGAGAGACTTTTCTTGCTCAAAGACATGGATCCACTCACAAACAGCATTCAATGGTGCTCTGAGTTTCCTCCCAACT
GTAATGACCTCCTCTGACTTGGGCACTTCTTACAGTGACTTTGAACTTCTGAGCATTAGAGATGAGTGAATCGCAGAAAACTAGACCCCTTGTT
AGATGGATGAGCCCAGAGAGGTTAAGAGACTTGCCCAGGGTCACACAGATACCACCCATATCAGAGCGCTCAGCTTTCCTTTCCCCTCCTCCCC
AGCTTGGACACATTCCTAGGATTTGGATGTAGCAATGTGAACCGTCCTGAGCAGTCCCTTCAAAACAGCACTTAGGCGAAAGAGGGGCTGTTCC
AACAAGACAGGACAAACACCCGTGTCCCTCTGCACGCTGCTCCACCCACTTTCTCATCTTTATATTACAGAAAAAGAGTCTCAAATAACCCAAA
CAGGCAGGGTATGATCTGCAGACAGTCTGCAGGCATCTGCCCAAGAAGCTGCCGQTCGGCTGGTCCATCCTCCTCTGATTTATGTTGTGTGTGT
TAAGCCTTAAATTGTTGAGTCTCTGGCTGGCTGCAGTGGTATTACTGGAGGGCTGGTGCATGTTTTCGGCTCACAGAGCTGGCTCCGGTACTGG
AGGTGTTGGCCCCGAATCAAGTCCAATCTCACACCTGTCTGAGACAATGAGGGCCTTGTTAGACACATCTCCAGTCGAGGAGGTGCAGGCTGCT
TATTTTTCACATTCAGAACCAGAAGGAGGGGGGTCTGGGACAAGGGGGATGGGAGAAGCCAAGGGCAGACGGGAAATGCTGGCGTCAGCTGCCT
CTCGCCCCCTTTCCCTCTCCTTCTCCCTCCTCCCTTCCCCCGCTCTTATTTAATAAACTTCTCCTTACTTTTGGAAATTAAAAAACAAACAAAC
AATACTAACAGTTGCCCTCTTATGAAGGATAATTTCCTACTGCAGAACAAGGAGCTCGTGGCCTGTTGTAGGATAAATTCCCAGTCATCATGGG
AGTGGAGCAGCCCAGGGGAGGAGTGGGGTGAATAGGCAGGGGCGCAGCCCCCACCCCACCCCAGCACTGAAACTTGAGCTCTTTGCACACTGAG
GGCGCCTGTCAGTGAGAGGTTTTCTCTTCTGGTTTTCCCTCCACTGCTGACATTTCAGTGACAGCCGCAGAAACTAGAGGGGACTTTGTCCCCA
GCTCCATATTATTCATTGATGAAACCGCAAACTGTTCTGTCCTGGTTCAGCCTTCCTGTCCCCTGTGATCAGAGCAGACAGAGCAGGCCACCAG
GACTTACGTCTGCAGACACTTTGCAAATCCAGTTCTCAAGAGGCCCTGACATGAGGCGGCCCCGGGCAGGAGTCCTTCTGGGTACAGGCTGGCA
CCCCTCAAGGGTGATCATCGCTCTGAAGGATCATTACTTAGTCAAACCCACCCTGAAATGACTTTCTATTGCAGGACCACACGACGTGGAGGCC
CATAGTGTAGGAGTCAGGAAAAGAGCTTCCCAGACTTATCAAAAACCACTCTTTACTGCGAGAGCCAACAGTAAGGAGCCTGTGTAGAAGAGCT
AAGAAAATCTCACACATGTAGTGGCCATGTCTTGGGGGCTTGAGTAACTCGACAAATCTCCCAGCTCTGTTTGTGATCACGTAGGCTCTTACCT
GAGCCGGGAGGCATTTAGGTGCCAGCAGTCACTCAAACAAGGAATGGGCAGCCCTATCCTTCTCATCCATACACAACTTATTTAAGCACTAAGC
CCCATTGCTGACTCCTAATTTGGCCTCAGCGACACAACAGGGAAAATCCATATGCTGTTTATTTCATTTGAGACTGAGGACTTACCAGAGATGA
GGTTGCGCCAGTCTGTTTCTTGTAGTATAAATCATTTGTAGCTCAGCCACCCTTGTATCTCTGTATGTACATCAGTTGACAATCCCAGAGAAAC
AAAAGGACAACTTTTCAGAGGCAGCAGAGTGACACACCGTGAATAGCAATGAGCATAGGCAACCTGGACTTCTGGCTACACTCCAGCCTGAGTT
TGAATCCCCTCTATGTGACCTTGAGTGACGTGTGAAGACCCAATGTGCTCACTGTTGATACTTTCCCCTTCAGAGAGTCCAGGGACTGCACTGC
ATAGGTTCCCTTAGCCCTGCACTCCTGGTAGAACCTGTCTGAATATGTCACCTTTGGAGGAAAGTCGCCGAAGAAGCAGGTGTTGCTATTTCAG
CTCAGCTGATGCCAGCGAGCCCTGATACCTATGGACACCATGGAACCACCAGTTTCCTCTCTCCTTCTCTAGCCATGCCTCCCTGCCTTGTTCT
GGGCCACAAGGTGCTCCATTCTTCCGAAGCTCATTCTTGCTCTGAAATGACCAAAAAGTCAAGCCAACCAACAGCAGGGACCCTCGACACTCAG
CTGGGTGAATGCTGCAGATCTTCCTGCTTGATCTCTGAACACCCATGACCAGTCCTGCCCACAAGCTTTGGTCCATGCTGGATGCCTACCCAGA
AACTTCCTTCCTCTCCTCCCCACCACATCCCCGAATCCTGCCATTCTTCAGGGCACCTCACCTGCCCCATCTCCTCCAGAACATCTTTGTCCTC
ACTGAGGCCCAGACTGACTCCCTTCCCGACTCCTGACTGATCTGTTGGTCTCTGTCACTTTGGCATTTCCCCACACATTCCCTTCTGCTGTTAC
CAGATAGGCTGCATGTTTGAGTGACGCTTCACCAACTGTCATGGCTATGATTTTCCACCATCTGTGCTGTAACACAGTAAAAACTCAACCACCA
CCACCGAGATGCCATCCAGGCAGGCAACCCTGAGCGTGCCCCCACCCGCTACACACTCTTCTCCCCAGAGCACATCAGGCTTCTGAGATCAACA
AGTTGGAAGGAGACTTCCACGTGGGCAGGAACGCTCTCTCCTTCCCAGGACCCCCACGAACCTAGCCCAGAGCTGCAGAGAAAGGGGTGCATGA
GTCGTTGAGGAAGGATTTTTCCTTGCTCCTGGGACAGCAAGTCAAAACTGTTTGTTTTAGGTTTTAAGTTAATTTCAGAATATCCTCCCCTAAA
CAATTTCTTTCCTTACATAGCACCAACTCTCTGCTGGGGCCCTTTGGCTGTTTCCTTTCTCCATTCTTTCCTTCTCCTTTCCCAGATCCCAGCT
GGGAACCTGGGGGGAGAGCCTGGCTGGCACCACCTGCCCTTTCTTGGGCCATTTCCCTTCTTGTTTGTGATGAGGACGAGCCTAGGAAGTCTCC
ATGCTTCTTGGCTTTCTGGCCCTCATCACTTTCACAGCCTTCTGCTAACTAAGCTAGTGAGCCCCCCTGCCCTTTCCCATTCCCTCTCTGAGGC
CAAACTCTCTCATCCCAGCTTCTTTCTTTTCATCTTCCTTCCTCTAAAGACTGCTCAGCCTCATCGGAGGCTGCCTTCTGAATCCTGACCCTGC
TTTCCACATTTTGTAAGACATCTCGGCCCCAGAGTAACAGTGCATGATTTACCTACCACTTCCTTGGATCCCAACCCCTACTCACTGCTAGCTC
CTGGCAGTGGCGTTCTTCCTCCTTCCTTGGTGTGCAGATGTGCCCCTGTGTGGCCTCCTTCTCTAAAAGCATTCTACATTGTTCTTGGTTCCAG
CACTTCTGAAACCCATCCTTCCAAGTTTACTCTTTTTCTTGCTATCCCTCGTATTTTCCCACTCCTCATGGAAGCTTCTTTCCCACACCCACCC
TGTGCCCTATGGCGGCCGCTTCAGGACCCCCAACCCCAGCCCTGGAGCGTTTCAGTGCTGCCATCCTGCTGGCTTCACTTCGTGCTTCCTCCAC
AGCAACTACAAAAGCCAAGGAACCTTCCCCAGTCCCTCCCCTCCCCACCATTCTCCTAGCAGAATTACATTTTAATTTTCAGCTGAATGCATGA
AGAACCATTCCAACACCTCTGTCTCACCCACAGTTCTCTAAAACACACAAACAAAAAAAAGTCTGGGTCAGCAGAGGGGAGACCATGGTCACTC
TCCTTGTCAACCTTCCCTTTGCCCATTCTTTGTTTTCTCTTCCCTCCACCTCTGTTCCTCTGAAACCTCTTTTCTTCAAATCAAGGGGAAGGTA
ATTCAGAGGTGCTGTTGAGACTGGTTTGCTTTTTAATTTTTTTTTTCAAAGAAGACGGGAAGTTCCTGTTTTTTGGAAAGGTTGTTCATTTCCA
AAGAAGTTACACAGCCAGTCAGCTATAGGATGATTCCTGGCTGCATTTTTTCGGGGTAGTACACAATACAAAAAAATAAGTCAATGAGACAGAG
AGAGTCAGTCAGTCAACACAAAGGGAGATGTTATAATAGGCTTTTGTTGGTACTTGCATTTTCTTGTTGAAAAACAGAGCAAAGACCCCGAGCT
GCTGAGGTGTGGACAGAAGCCTGATGTTATCAGGCCCCCAGAAATCCTGCCTGCAGTCCAGTGGCACGCAGGATCCAGAACTGGAAAAGACGCA
GGCAACTGGTCCTGCTCAGCACAAACGGTGCTTTTAGTCTTTCGTAAACAGCCTGTGCCCCACGGCTCAAATCCTTCAAAAGTTTGCCTGGGTA
ATTGCATTTCATTGCTGTCTGGCATGTGCTCATCAGTCTAATCTGGAGGTTCAGACAGCCAGGCCAAAGCTTGCGAGACTGGAGGAGAGCGAGC
CTGTGAATTTAATCCACTTGAAACACAGAATAATACAACTGCATCTAACTCATTATTTTAAGTGATGCTAGTCCAAAAGGGGATGGTGCTTTCC
ATAAAAGCATTTAAAATCAGCACGCTGCAGTTGCAGCCACTTGGCAGTTTAAGGCCATTAGAAGAAAAGGCCCACACAGGTGACAGTGTGATCC
CTGTCGCCGTCTGCTTGTCCCTGAAAGCCTTCCTCCTCCCCACGACCACCTCAAAACACAGAAGGCAAAAGGAATGGTAACTTCCATCCCTAAT
TTTGACTTTGCGTTATTTGTACATTTACACCAACAGACACCAACTTTTATTTGAGATTTCCGGTCATTTTGGCATCGTTTCAGGGTGGAGAGAA
GACATAAATGAAGTTAGGACTTCCCACTCCATCTCACCAAGCTCAGGATCTCACAGAGCTATAAGCAGGCAAACGTGTTCCCCACCAGAACCAT
CTGCACAATGTGCTTTCCAGTGACTTCTTTTTTTTTGAGGTTTACCTAGGTCTTTTTGTTCTCTCAGTACATGGTAACTAGATTGATGGCAGGG
TTATAGTTTCTAACTATCATGTAGTTAAAGCACTAGTCTACAAAGAGCCACAAGGTCTGTCCCTGCTCTGTTGCTATCTGCTGTGGAGAATAGG
GGGAGTCCCTTCACCATTCCGCACTGCAGCTTTCTCTGAGATGGGGCTGGCATGGTTGTCTCTGCTGTCTCGGACGTTGCTTTCTGCTCTATCA
TTCCATAAGACATTAGAATGAAACCTTAACTAATGTTGACGGTGATTTGCGTCATTATCTAGTTACATGTGACTCATGCCCAGGAAGCCCAGGA
GAGGCAAGTGCTACCCTGTACCCTCCCAGATGGCAAAGCCGACGGTCAGCTCAGGAGCAGTCTTGTTGGTAGTTCACACCTCCAGCTCACCCAG
GGCTCAACATGCCAGCTCTAAGTCTGGGTGCCAGGCCATGAGACTGAGAGAGCAGTCGCCTTAAACCTTACATCGGGATGGAGTTTCCCCCTGG
GTGCATCATGACTGCAGCCACCCAGGATTCTCACAAGGGCTCCAGAAAGTGCTCACCACACACCTCATCTCCTCATACCCCTCAGTTCATTTGT
ACTGTGCCCAGGGGACAATGCTGTGTTAGCTTTCCCCGATCCTGAAGAGCACCTCTAGAAGTCACATCTTCCGTCTTGTCATCATGGTAGCTGA
AACCATTGTGGCAGTGTGTGCTTTCCCTGAGCAAGGTTTTCTTTGACACTTTAGGGCACGCTTGATCTTGTGGGCAGGATCATTCTCTGTTGTA
GGGGCCATCCTGTGCATTGTAGGATGTTGAGCAGCATTCCTGGCCTCCACGTACTAGATGACAGTAGCACTCCCCCTTCCTCAGTTGTGGCAAC
CAAAAATGTCTCCAGACATTGCCAGAAATCCCCTGGAGGAGAAATCACCCGTTTGATGATCGCTATTTAAGAGTAATTGGGAAATTCACAAGGA
TCTTCTAGTCAATATACACAGGATCTCAATCCCTGAAAAAAATGTGACTTACTTAGCTATCTAAACTTGGTAGTAAGTCAATTTTGACCCTTCA
GTTCTGTAGGTCCTAGCATTCCTGTCTTCCATAGGTCATCCCTCTTGTTGACCAAGAGTTTCATAGTTGTCTGCTATCTCAACCTCTACATTTA
ATTTTTTTAAAAAAAATTACTCCATTTTAGCCCAAGTATATGGCTTCTATGAGAAAGCCTTCCTTGGCCTTGTAAATCAAAGCAGTTCTCTGGA
CGGAGGTTTCCCACTGCTGGGCTCAGTATCATCTTTCTCAGGGTGACCTCATCTATTCAACTATGAAGGTTGTATTTGAAATGCCATTTACTTT
CTCTAGTCAATAAAACGAAGCTGTGGATCTGTAGATCATCAGAGGTCTGCTTATTACCAAGCGGTTTTGTGTGTGTCATTTTTTAAATCGAGAT
CTGTGTTGGAGTTTAAAAGAATTCTGGCAGATTTCCATTTGCCTTTCTGAATCAGAGTTTGGGGTCAGCGAGTGTAGATTCCCCCTAGATGAAT
CTGACTTTATTAGTTTGTGTCCTTTGTTCAGAGCACCAGGGATGAGCTTATGAAAGCACAAATGGCTCTTATCTTCAAGTGTTTCCTTTATGCT
GCAAATTCCACCTCACCAACCTGTGTTATTAACCCACTGCAATCCCAATTAGTTCACATCAAGGTCAACTCTTTGGGGTGTGCAAAATGGCTGC
AAACTTAACTGAACAAATTCCCCTTGTGTAGCTCTCACATGAAGGTGGGGGAGAAGACTGCAGCAGGGGAACTTAGAAGAAAGAGGGAGTTTTC
TGATACCATAGAAGAAAGTGATAGTGATTCCCCTTGGCTCACAAGCCAAAGAAATCCAATTATTCCAAATAATTCAAAACTTGCATAGGAACTT
GTCACCAAATGAGGGAACATTTTAGCCTCTTATTCACTCTGTTGCTTGGTGGCAAATATTTAAGAAATTTCAAAAAATAGAGAGGTGCAAGAGA
GCTATTTCTCAGGGGCATGGAGGTTTCTTGGGGGCATTTCTCCCGGGTATAGAGGGCTGTTTCTTGGAGGCTATTTCTGTGCATGGAGAAGCCT
TGAGTTTCAGAGGTATAATAAACACTACTCCTCCAGAAATATGTCTGTGAAAAATAAGCTGGAGCGGGTGGGTCTGCAGGACACTGTGTTTAGG
AATGAAGGCTGCCTCTCCTGCCATCTCTCAGGAGCTCCCATCAGAACAGACAGTAAAGACAGGAATGGCATGTCTCCAGTTGTGGGGCAAGGCC
CCTCCTAGTGTGAAGAAAATATACTTCCTGGATCTATCATTATCTCTAGCATATCAGAGGGGGGAATAAAAACAACTGCGGTGGCAAGGCCCTG
CTGAAAGAGAAACTCATTGTTCTGCACATTCAGAGACTTCCTAGTTGTAAAGCCGTTCAGAGCTCTTGGAGGTGAAGGGGCAGCCAGGGAATCA
CATAGAGTGAAAGACCTGTGTCCAGCGGCCAGGTCAGAGGGCGTGTAGAAGACGACATGAAGCATTCCCACAGGGCAGTTCCCTGAGGTTCTCT
CCTCCAGCTCTTGCTCCAAGGAATTCTAGAAGCCTTGCCCTGATGTGTGCGAAAGGAAAACAAGGTGAACAGGATGCAGGTAGGAAAAGAAGAG
CTGTGACAAGGTAGTCAGATGGGAGGACTGCATGAGAAAAACAGAGGAAGAGAGCACAGAAGCAGCCCTTTGATTTCACTCGCGTTTATTGAAG
GGGACGAGGCACTTCCACTTGGGGAATTCTACTTCCCTTTCTCAGGATATCCTTCAAATTGCGAGCATGGAATGGAAGCTCTTAAAGAAAGAAC
GGTTGCCATTCATTGTGGQTCACACTTCACAAGACAAAGAGCAGTGAATCCTCGATTTGTAAAATGCAGCTTTCAGAATCTGTGTTGTGGCTGG
CACGGTGCAACACTGCTGTGAGGCCCTGACGCAGCTGTGTTCTGCAGGGTGGCTATGGTCCTCCTTGTGAAGCAGGAGCCTGCTGCTCTGCAGG
AGGAAGGGCTTATGTTGCATCCTAGCGGTGCATGCTACCCCGCTGGAATAGTGCACCTTGGAACTCTGGCTGCCTAAGCAAGGTCTCATTTCTG
GTGGCTTTGTGAGACCAAGCCTCTCCCTCTTCTGTTACCTGGAGCAACAACCAGCTAAGCCAACAGTACTCTCCAGACACTGTTATCCTTTCCT
GGAAGGGCAGGGGAGAGAAGACTGTTTCCTGCCTTCAAGGTGCCAGCTACTGCATTGACAATATCCGGCAGAGGGGCACATTTCACCCTCCCAG
GAATCTAAGGGACAGATGCAATAACATCTCCTTATGGGGGGAAACTGAGTCTCCAAGGCATTAAGTTACTTGCTTCAGCACACTGTGAGTGAGG
GGCCAAGCTAGGGTCTCTGTCCTTACCTCTCAGACTGCAGAACTATGCCCTTATTGTATCCTTACTGAAGCCCTTTTGGCTTTTAGAGAGACAG
CTCTGGGATGAGGTTCCCAAATCCTGAAAATCTTTZGGGCCAGAACAAACAAAAGCATCTTTATGTTAAGCCATATTTCATGTTTATTGTTTTG
AATTCTCCTTTTCCCCCATTCATATGAGTTTTTCTTCTGATTTTATTAGAATGCCATGCCTCAGAGTTTGCTGCTTAAAACCAGAATCCCTGGC
ATAAAATCCAATAAGCCTGCCTTAGGCCCCATGTCATATGGAAGCAGAAGACAACAGAGACTGCTTGCTGGGCTTCAAGCCACTCTAGAATTTT
TCAAACAGTTCTAACATTTTACAACATCAAAAAAGTGCCAAAGTAAAAACTAAGGCTGCAGCTAGGAGAATGCCCACTGAGAAACCCGTGGTTG
GGTTTGTCTAGAGAGGAAGGCTTTTGAGATCACTGCTCCATGCCCCCTCGCCCTCTCCCACCAGAGGTCTGGCTCAGGGACAGCACTTGGGCAC
AGAGAGCTGGGGCAGTGGCAGAGATGGATGCGGTAGTCACCAGACGAAGCCCAGGCTGCAAACTGGCATCACACACACCTCTCTCGAACAGATC
TCCATCTGAGCTGGGCTCAAAAGCATCGTATTCTCCAATATCGACAACTCATAGGTTAGGGGGTTCATCATTTTGTAGCCTCTGCAGTCCCAGA
CGTAAAGTCACTTCTTTTTTTTTCTTGCGTGCTGACACCCTAAGGCATTACATCGACCCTGGCAAATCTCAGCCATGTTACTTAACATGATCTG
CCCCAGCGACAGCTCTTAACTGTATCTGTTTACCCAGAGATATCACAGGTGCCTCTCTACGAAATCCCACCCGCCCCTCATCACCACAGTCGAC
ATTGGCTGTAAAGCACGGCCTTCCATGCCCAGCCTGGACTGCAGGTTGGAGATGACAGTCTCCCAACCCAGGCCTTCCAAAGCATAGACTATCG
GCGTGCACAATCCTCTTGATATCAGGTGCCACCTCAATGGAGTGTTCTTCAGAAAATTCTGTTTCTTTAGCTGTGTGCAGTCCTGGGAGATGCT
GTGCCTCTTCTGCTTGAAGCTTCCCAGCTCCCACTTGAGGTGTCAGTGATTTGTTTTACTGGAGTGAGTATCCTCATTCTCCACCAAGGGTCCA
GTCTCAGGATGGAGGCTGCTTGAGATATCCCAGCCCTCAACACAATATTTCTGGCTCATGTTTTTCAAGGAACTAAGCGGGGCAAAGGTGCCAG
AGGAGTAGAAGGGAGCAGAAAGTTGTCTTTACCCTCCTGGAGAAGAAGGACATGGAGGAGTCGAAGGAAATAAATAGGGTGGCCATAGAAGAAT
CTAAACATCCATGATGTGCTGGTGTTGCAGGAATGTACCCAACCTGTCTGTAGAGCTCCTTCAGGCCTGGAAAGTTGTATACGGGGGGCAGAGG
TGCAGAAAGTGCACAGTGCACAAGATCCTTATGGAAAAGATGTCTGCAACAAATGTTTATTTAACAAAGTACAGACAAAGAATCTCTCTGCAAG
GGAGGAGGCACCAGAAACCAGAAGGTGCCTTCTGTTCCCTCTGAACTATGGTCCCTCCAGCTGTGTGCACTGAAGGCCACTCTCCCTCTGCCTC
AGGGACACGATGCCAACTGAGAATGAGACATGTTCTAAAAACACCCATCTTTTCTGGAAGCTAGTGACCTATCCTTGAACCCTCCCTCCAAAAG
CACTCTGCCTAAATACGTAGCCCTTTCCCACACGTGTCTCAGACTCATCTGGCTGAGCGTAGCAGCCTCGTGGTGCAGGGTCAACTGTTTCCCC
TGGGAACCGTCTGACAGTGCTAATGAGCAATTGCATTTTGCGTTTATAGAGCACCTTTCATCTGTGGATCTCAAAGCTGCCCCACAAACATTAA
TTAACTCTCACACCTGTGAGGCAAGTGAGCCTGGTTACCCCCATTTTTAGAGGTGAGGAAACAGAGGTTTGAAAAGAAAAGGACTCCAGGGTGA
ACCAGCAGGGTTGGGGGCGGCCTGATGTGAGGAACCAGCATTCTTGATTCACACGGCCCTGACCACAAAGCCACAAAGCTTCCCCGGCGAGGTG
ATGAATACGGGTCCCGGAAGCAGGGAGTGGTGGAGAGGCGGACACAGGGCTCCCTTCTTCCTTCCCGCAGGCAGCCTGCCACCTCCCAGGCTGG
CAGCTCGGCAGTGAGGATCGGATCCCTCAAAACCTGAGGCCTTTAAAACAGCATACCCAGGCAGGGTTCTGCCCCGGCTTGGTCTGTCTGCTTC
TCTTCAAAGTAATTGCTGGAACAACGAATGCATCTGAGCTACACATGACTAACCAAGATGTCTGCAGCCGGCCAGAGACGAACGGTTGGCAAAT
CCTGGAGCTGTTTCACAACCTCCCTTTTTTCCAGGATCCAGAGCTGGTGCTGACTGACTCATGTTATCATGGGATTTCAATCATAAATAGCGGG
TGTGCTGACCCGGTGTCTGCTACCTGGGGGTACCTGTAGTAGGAATAGCTGACTGCTCTTCTTACTTGTGCTGTGCCGGAGCTTTGCTCCCTGC
TGCAGAGGCCAGAGGAAACACTGCTTCTCAAAGACAGTGGAACTTTCAGGGAAATGTCCCTTTCAGAGCAGAGTCACCCACCCCCAGGGACTCT
GAAAGGCCCCTGACATCACACACCTCTGACAGATGTGATGCTGGGAAGCATCTGGGGTCACCCCCTTTGTCTCCAAGGGGAGCCTAGGCCTTTC
TTCCCAGGGGTCCCACACAGTGAGATGCAAACCACCACTGGGGCTCCCCCTAACACATCACCCTGGAAACTGACTCACATCTCTGAGTCCTGAG
TCACATCCTGTTGTGTGTTCTCAGCTCTCCAAGACGGCATTACACCAGCTCCCCATGACTGAGGTGAATCTGGAGGGAACCCTGGGAACGTCCC
TACCTTCCTCCAGGATGAAGGAGGGAAAAAATTCCTCCAGGGAAATAACCAGGGCTGCCCCTAGGGTTTATATGTTCACCAAGCTGATTTGCCT
TTTTCAAAAATAGATGGTAGAACCCTGTGGTCTATGTGACTTTTATTTTTAAATAGCTCTGAGAGATGACTGAAAGACCTGGCGTTTTAAAGAT
GTCAGCCACACAATCAAGGAAGGGGCTTTCTAACTATAACCTTCTCCAAGCCCAGGATGAAGGGTGCCACCGCTCAAGGGGTGCTGTTGGAAAC
CCAGCTTCTCAGGGTGGGTGTTTAAAAGACCCTAGAGTGGACAGTCAGAGGAGGGCCGGGAGGGGTCTCTATCCATCTTCTCCCAGCGCCTGTG
GGCAGCACGAGGACAAAGAGGGATGTTTTGAACTTTTCAAAGAACAAGATATTTTCTTCTATTGATTTTGTCTATTATTTTCCTGTAACTTGTT
TCACTGGTTCCTAGTCTAATTTTTATTATTTCTTCTCTTCTGCTTGCTTGAGGTCTGTATTGCTATTTTCTCCAGTCTCCTGAGGTGGAAGCTT
CAGTGACTCATTCTGGATCTTTCTTTTCTAATATATGTATTCAATGCTATAAATTTCCCTCTAAGTACTGTTCTATCTGCATCCAAAAATTGTA
ATAAGTTGTATTTTCATTTAGTTCAAAACATTTTTTAATTTCTCTTCAGACTTTTTATTTGACTCATGTCTAATGTAGAAGTATGTTGGTTTGT
CTCCAAATATTTGGAGATTTTCCAGTTATCTATTATTGCCTTTTTAATTTAAATCCATGTGGTCTGAGAAAATACTTTGTATGCTTTCTATACT
TTTAAGGATCTTAAAGTGTGTTTTGTGGCCCAGAATATGGCTTATCTTGGTGAATCTTCCATGTGAGCTGAAGAATGTGTATTCTGCTGTTGTT
AGAGTATTCCATAAATGTTAATTAGATCAAGTTGACTGGTAGTGCCGTTTAGGTCAACTGTGTCTTAATTTTCTGCCTGCTTGATGTATCAATT
ACTGAAAGAGGGTTGTTGAAGTCTCCAATTGTAATAGCCTATTTACCTATTTCTCCGTGTAGTTCTATCAGTTTTTCCCTCATGTTTTTTGACA
CTCAGTTTTTAGGTGCACATATATTAAAGATGCTGTCTTCTTAGAGAATTCACCCACATTATCATCATGTAATTCCCTTCTTTATCCCTGAAAA
TTGTCCTTCCTCTGAAGTCAGCTTTCTATAAAATTAACATCCCTCCTCTGGCTTTCTTTCAATCAGTGTTAGCATGGTGTACCTTTTTCCATCC
CTTTACTTGTAATCTGTATAAATATTTTTATTTAAAATAGGTCCCTTGTAGACAATATGTAGTTGGGTCTTGTCTTTAATCCACACTGCCAATC
TCTGTCTTTAAATTGGTGCATTTAGAGTATTCATATTTAACCTAAGTATTAATATAACTGCAGTAGTATCAATCATATTTATAACTTTTATATT
TGTTGCACCTGATTCTTCCCCCCACACCTCTTTTTCTACCTTTTATACGTTTAACTGAACATTTTATATGATTCCATTTTATCTCCTCTGTTAG
CATATTAATCATTCTTTTTAAAAATGTTTTTAGTTACTGCCCTAAAGTTTGAAAAGTACATGTATAACTAATCTACATCTAGCATCAAATAACT
ACCACTTCTTCCTTCCTGTTTATATCATTACTGCCTTTTATTTCATTTATCCACATGCTATAATCACTCAATACTTTGTTACTATTATTGTAAA
CAGTTATCTTTCAGATCAGTTAAGAAAAATAAAACTTAATTTTACCTTAATATAGTACTTTTCTAATGCTCTTCCTTTTTTATGCAGTTCTTTT
TGACATTTCTCATAGGGCAGGTCAGCTGGCAATGGATTATTCCAGTTTTGTTTGTCAGGAAAATATCCTTATTTCTTTGAATTTGAAGGATGTT
TTGCTGAATGCAGAATAATAGTTTGGTAGCTTTTTTGTTGCAACACTTCATGTATTCTCCTTTCTTTGTGTTTGCATGGTTTCTGAAGAGAAAG
ATAATGTAATTCTTATCCTTTTTCCTCTATGGATAAGGTGTTGGCTTTTCCCCCTCTCTAGCTTCTTTCAAGATTTTCTCTTCTCTTTGGTTTT
TTGCAGTTTAAATATGATATGCCTGGGTGTAGATTTGGTATTTATTATGCTTGGTGTTCTCTGAGCTTTCTGGATCTGTGGTTTGGTATCTGTC
ATTAATTTTGGAAAATTCATAGCCATTAATACTTCAAGTATTTCTTCCACTGTTTTCTTTTTCTTCTCCATCTGGTATTCCAATTACACATTAC
ATCTTTTCAAACTTTCTCACAGTTCTTCGATGTTCTTTCTTTTTCCTCTTTATCTTTTTGTTTGGTAAGTTTCTGTATCTTCAAGTTCATTGGT
TCTTTGCTTTGTCATGTTCTGTCTACTAAGTAAGCCATTCCTCATTTCTGTTAGGGTGCTTTTGCTTTTTAGAATTTGCTTTTGAGTCTTAAGA
GTTTCAGGCTGCGCACGGTGGCTCATGCCTGTAATCCCAGCACTTTGGAAGGCCGAAGTGGCTGGATCACCTGGGGTCAGGAGTTCGAGACCAA
CCTAGCCAACATGGTGAAACCCTGTCTCTACCAAAAATTACAAAAAAAAAGAAAAAATAGCTGGGCGTGGTGGCAGGTGCCTGTAATCCCAGCT
ACTCGGGAGGCTGAGGAGGAGAATCACTTGAACCCAGCAGGCGGAGGTTGCACTAAGCCAAGATTCCGCCACTGCACTCCAGTGTGGGCGACAG
AGCGAGACACTGTCTCAAAAAAAAAAAAAAAAAGTTTTCCATCTCCCTGCCTACATCATCCATCTCTTCTTGCACCTTGTCTTCTTTTTCCACT
AGAGCCATTAACATGTTAATCATTGTTAAATTCCCTGTCTTGTAATTCCAAAATCTGTGTCACATCTGGGTCTGGTTCTGATGCTTGCTTTATC
TCTTCAGACTGTGTTTTTTTCTTGCCTTTCTTGTAATTTTTTATTGAAAGTTTAACATGATTTATCAGGCAATAGGAACTTAGTAGGCCTTTAG
CGTGAGTTATTATTCTCATGTACCCAAGAGTTGGCCTGTATTTAATCTTTTCAGAAGGTACCAGAGACTTCAAATTCCTCTAGCGTCCTTGTTT
TTGTCTCCCATGTCATCTGTGAGCTTCTCTATCAACTCCTCCTTAGTCAGTATGTTGAAGCTCTTTCAGCCATAACCCACTGTTATAATAATAT
ATTGGATCCTGTGAGAATCTGGTGGAGTTCCTGAATGAAAAACCCACAACAGTGCACTTCCTCCTGAAACTGTAGCCCCTAGGAGTGTCTTATT
CGCACACTAGTCTGTACTGAGCCTCTAGCAATCTGTCAAAATCACCATTAAGTGTTCTCATCAGTTTATGAATTCAGAAATTCTGCTCCCAGCA
AGCAGACCTCACTATGATTCTCTATATTTCCCTGCTTCTCAAAATTTGGGGTTGCAGTTTGCCCTGCAACTTCAGCTTTCCATTGGACTTAAGA
AAAGTCATGGATTTTCAGTTTGTTCAGCATTTTTTCTTATAAAAACAAGAGTGATGACTCTCAAGCTCTTTAAATTTTGGAACTGAAACTAGAA
GCAACAGGACATTCTGACATGAATTATTTAAAAGGGTCATTGTTGACTCTAGACACATTTTAATATTGCTGCATCAAAAGATAAAAGCATTCTC
CTTTCTTCTTGTCTGATATATGCCGTGTACACCTCTGAGCTCACCACTAACCTCAACTCTACCTTATCCCTAACTTCCCATTTCAAATGGAAGG
GAATGGGGATCAAGGAGTAGAAGGCCTGTTTTACCCATTATTGATGATTAAAAAGCTGAAAATAACCCATCACAAAATGGCCTCACTAAAGTGG
CTGCATCAGACATCATTTAGCCTGGAAAACCACTATGCATAAAGGCTCAAATTTAAATTTTTGAATCCAGTGTTTTTTATTTATTATTATTTTT
TTTTTTCTGAGACGGAGTCTGGCTCTGTCACCTAGGCTGGAGTGCAGTGGCACGATCTTGGCTCACTGCAAGCTCCACCTCCTGGGTTCATCCC
ATTCTCCTGCCTCAGCCTCCCGAGTAGCTGGGACTACAGGCGCCCGCCACCATGCCCGGCTAATTTTTTGTATTTTTAGTAGAGACAGGGTTTC
ACCGTGTTAGCGAGGATGGTCTCGATCTCCTAACCTCATGATCCACCCGCCTCGGCCTCCCAAAGTGCTGGGATTACAGGTATGAGCCACTGCG
CCCCGCCCTGAATCCAGTATTTTTTAATAGAACATGAGTTGGGTTGAGATACTGAGTTCCTGCCTTCTCCATCCTAATCCCAAATT1CCAATCT
TTGGAAATTTGCCCCTTTACCATTTAAGCTCCTGACCCCAGCTCCTCTCCAGCCTGAAGAGTGTGTCTGTCAAGACTGTAGCTGTGAACAAAAC
ACTATTACTTGAGCCTTATCCTGGAGCAAATGTGTGTGGTCTCACCAATGAACATCAGATAAGTCCCTTGGCATGGATTTCTTTTCCTTTTTAG
TAATACAATCATAAAGGTTATTCAGACTTATTGAGCATTGACTATGGCCAGAAAGTGTGATACTTGCTTTCCATATAGTTTATCTCACTTACTC
CTATTACCCTTTGAAGTCAGAATTCTAACTATGAATCAGAGAAAATAAGTGACCTGCCTAAGGTCATCCAGTTGGTTTATGGTGAAGGCAGGAT
GCAAACTGAAATTTGTCTGTGATTTGAGTTCGGTGCTTTCCCTAGTCTCCTCCTCTCAGAGGCAGAACATAAGCATGTAATAGATACCACTAGG
GTTCTTGTTTCTACCTTGGGAACAGCTGTTTGCAAGTCCAAATTAGGCAGCTAATCTAGCAGTTAGCCCAGATACAGCCCTTGGGCACCCTAGT
AGCTGTAACTTCCAGAAGGCAGGAACCTTCCATTCACTAGTGAG1CCCCAAAACAGTGGCCCAAGTGAAGCAAAAATGTTTGTTGGAAATATGA
GTGAATGAAAAAGTAATTTCATGAACACATCAATGAATAAATGAATGAAGCAACACCCAGTATGATGATTAATGCCACAGGAAGATGCCTTTCC
AAGCATCACAAGATCCTGGGATCTTCACTGACTGTAAGTGCCTGTGGGGCAGGGACCATGTTGGTCTTATTCATCACTCTCCTGCCAGGTAAAT
TAGCACATTCCTGGCACACTGTAAATGTTCAATAGCTTTTCTTCTTTTAGATCCATTGACTTATCACAGGCAACTCTTTCAAGAAACAACTAGC
CCTGTGCTCTGGAGAATGATTTACAGTGGTTCTCTTCCTCAGCCTGGCGCCTTTTCTGGATAACTCCCATCCTACTCTCCTCTACTTCCTCTTC
CTAGAATACACAAATTTATTATTACAGGCACCACTCTTCACCCCACATCTGATTC
HUMAN SEQUENCE—mRNA
GCGGGCGCGGGCGGGACGGGCGCCCCGCGGCCGGACCCAGCCAGGGCACCACGCTGCCCGGCCCTGCGCCGCCAGGCACTTCTTTCCGGGGCTC
CTAGGGACGCCAGAAGGAAGTCAACCTCTGCTGCTTCTCCTTGGCCTGCGTTGGACCTTCCTTTTTTTGTTGTTTTTTTTTGTTTTTCCCCTTT
CTTCCTTTTGAATTAACTGGCTTCTTGGCTGGATGTTTTCAACTTCTTTCCTGGCTGCCAACTTTTCCCCAATTGTTTTCCTTTTACAACAGGG
GCAGAAAGTGCTCTGTGGTCCCAGGCGAGCCGTGAAGTTGCGTGTGCGTGGCAGTGTGCGTGGCAGGATGTGCGTCCGTGTGTAACCCGAGCCG
CCCGATCTGTTTCGATCTGCGCCGCGGAGCCCTCCCTCAAGGCCCGCTCCACCTGCTGCGGTTACGCGGCGCTCGTGGGTGTTCGTGCCTCGGA
GCAGCTAACCGGCGGGTGCTGCGCGACGGTGGAGGACTATCGTCTCGCTGCTGCCCGAGTCAGGCCTGAGTCACCCAGCTGATGTAGACAGTGG
CTGCCTTCCGAAGAGTGCCTGTTTGCATGTGTGTGACTCTGCGGCTGCTCAACTCCCAACAAACCAGAGGACCAGCCACAAACTTAACCAACAT
CCCCAAACCCGAGTTCACAGATGTGGGAGAGCTGTAGAACCCTGAGTGTCATCGACTGGGCCTTCTTATGATTGTTGTTTTAAGATTAGCTGAA
GATCTCTGAAACGCTGAATTTTCTGCACTGACCGTTTTGACACAATTCATTGAGAGAACACAGAACATGACAAGTACTTCTAGCTCAGCACTGC
TCCAACTACTGAAGCTGATTTTCAAGGCTACTTAAAAAAATCTGCAGCGTACATTAATGGATTTCTGTTGTGTTTAAATTCTCCACAAATTGTA
TTGTAAATATTTTATGAAGTAGAGCATATGTATATATTTATATATACGTGCACATACATTAGTAGCACTACCTTTGGAAGTCTCAGCTCTTGCT
TTTCGGGACTGAAGCCAGTTTTGCATGATAAAAAGTGGCCTTGTTACGGGAGATAATTGTGTTCTGTTGGGACTTTAGACAAAACTCACCTGCG
AAACTGACAGGCATTAACTACTGCAACTTCCAATAATGTGTTTGCTGATCGTTTTACTCTTCGCATAAAAAAATATTTTAGGGGTGTATGAGGT
TTTGCCTTCAGGAACTTTTCTAACAGCCAAGACAGAACTTACCTCTGCAAGCAAGATTCGTGGAAGATAGGGTCTCCACTTTTTAATGCACTGG
GCAATCGGTTGCTACGAGCCCATCCTGAGTCAGAGGCCGATCCGCAGAACCAGAACGTTTTCCCCTCCTGGACTGTTAGTAACTTAGTCTCCCT
CCTCCCCTAACCACCCCCGCCCCCCCCCACCCCCCGCAGTAATAAAGGCCCCTGAACGTGTATGTTGGTCTCCCGGGGAGCTGCTTGCTGGGAT
CCGCGCCCCTGTCGCCGTCTGGTACCAGCTGTTTGCACGGTCCTAACTCAATCGGCTTGTTGTGATGCGTATCCCCGTAGATGCCAGCACGAGC
CGCCGCTTCACGCCGCCTTCCACCGCGCTGAGCCCAGGCAAGATGAGCGAGGCGTTGCCGCTGGGCGCCCCGGACGCCGGCGCTGCCCTGGCCG
QCAAGCTGAGGAGCGGCGACCGCAGCATGGTCGAGGTGCTGGCCGACCACCCGGGCHAGCTGGTGCGCACCGACAGCCCCAACTTCCTCTGCTC
CGTGCTGCCTACGCACTGGCGCTGCAACAAGACCCTGCCCATCCCTTTCAAGTGCTGCCCCTAGGGGATGTTCCAGGATGGCACTCTGGTCACT
GTGATGGCTGQCAATCATGAAAACTACTCCGCTGAGCTGAGAAATGCTACCGCAGCCATGAAGAACCACGTTGCAAGATTTAATGACCTCAGGT
TTCTCGGTCGAAGTGCAACAGGGAAAAGCTTCACTCTGACCATCACTGTCTTCACAAACCCACCGCAAGTCGCCACCTACCACAGAGCCATCAA
AATCACAGTGGATGGGCCCCGAGAACCTCGAAGACATCGGCAGAAACTAGATGATCAGACCAAGCCCCGGAGCTTGTCCTTTTCCGAGCGGCTC
AGTGAACTGGAGCAGCTGCGGCGCACAGCCATGAGGGTCAGCCCACACCACCCAGCCCCCACGCCCAACCCTCGTGCCTCCCTGAACCACTCCA
CTGCCTTTAACCCTCAGCCTCAGAGTCAGATGCAGGATACAAGGCAGATCCAACCATCCCCACCGTGGTCCTACGATCAGTCCTACCAATACCT
GGGATCCATTGCCTCTCCTTCTGTGCACCCAGCAACGCCCATTTCACCTGGACGTGCCAGCGGCATGACAACCCTCTCTGCAGAACTTTCCAGT
CGACTCTCAACGGCACCCGACCTGACAGCGTTCAGCGACCCGCGCCAGTTCCCCGCGCTGCCCTCCATCTCCGACCCCCGCATGCACTATCCAG
GCGCCTTCACCTACTCCCCGACGCCGGTCACCTCGGGCATCGGCATCGGCATGTCGGCCATGGGCTCGGCCACGCGCTACCACACCTACCTGCC
GCCGCCCTACCCCGGTTCGTCGCAAGCGCAGGGAGGCCCGTTCCAAGCCAGCTCGCCCTCCTACCACCTGTACTACGGCGCCTCGGCCGGCTCC
TACCAGTTCTCCATGGTGGGCGGGGAGCGCTCGCCGCCGCGCATCCTGCCGCCCTGCACCAACGCCTCCACCGGCTCCGCCCTGCTCAACCCCA
GCCTCCCGAACCAGAGCGACGTGGTGGAGGCCGAGGGCAGACACAGAAACTCCCCCACCAACATGGGGGGGGCCTCCTGTTCGCGACAAGCCCG
CCGGGATCCCGGGCCCTGGGCCCGGACACCGTCCTGGGGCCGAGGGCGCCCGACGGACAGGATCTCGCTGTAGGTCAGGCCCGCGCAGCCTCCT
GCGCCCAGAAGCCCACGCCGCCGCCGTCTGCTGGCGCCCCGGCCCTCGCGGAGGTGTCCGAGGCGACGCACCTCGAGGGTGTCCGCCGGCCCCA
GCACCCAGGGGACGCGCTGGAAAGCAAACAGGAAGATTCCCGGAGGGAAACTGTGAATGCTTCTGATTTAGCAATGCTGTGAATAAAAAGAAAG
ATTTTATACCCTTGAGAAAAAAAAAAAAA
HUMAN SEQUENCE—CODING
ATGCGTATCCCCGTAGATGCCAGCACGAGCCGCCGCTTCACGCCGCCTTCCACCGCGCTGAGCCCAGGCAAGATGAGCGAGGCGTTGCCGCTGG
GCGCCCCGGACGCCGGCGCTGCCCTGGCCGGCAAGCTGAGGAGCGGCGACCGCAGCATGGTGGAGGTGCTGGCCGACCACCCGGGCGAGCTGGT
GCGCACCGACAGCCCCAACTTCCTCTGCTCCGTGCTGCCTACGCACTGGCGCTGCAACAAGACCCTGCCCATCGCTTTCAAGGTGGTGGCCCTA
GGGGATGTTCCAGATGGCACTCTGGTCACTGTGATGGCTGGCAATGATGAAAACTACTCGGCTGAGCTGAGAAATGCTACCGCAGCCATGAAGA
ACCAGGTTGCAAGATTTAATGACCTCAGGTTTGTCGGTCGAAGTGGAAGAGGGAAAAGCTTCACTCTGACCATCACTGTCTTCACAAACCCACC
GCAAGTCGCCACCTACCACAGAGCCATCAAAATCACAGTGGATGGGCCCCGAGAACCTCGAAGACATCGGCAGAAACTAGATGATCAGACCAAG
CCCGGGAGCTTGTCCTTTTCCGAGCGGCTCAGTGAACTGGAGCAGCTGCGGCGCACAGCCATGAGGGTCAGCCCACACCACCCAGCCCCCACGC
CCAACCCTCGTGCCTCCCTGAACCACTCCACTGCCTTTAACCCTCAGCCTCAGAGTCAGATGCAGGATACAAGGCAGATCCAACCATCCCCACC
GTGGTCCTACGATCAGTCCTACCAATACCTGGGATCCATTGCCTCTCCTTCTGTGCACCCAGCAACGCCCATTTCACCTGGACGTGCCAGCGGC
ATGACAACCCTCTCTGCAGAACTTTCCAGTCGACTCTCAACGGCACCCGACCTGACAGCCTTCAGCGACCCGCGCCAGTTCCCCGCGCTGCCCT
CCATCTCCGACCCCCGCATGCACTATCCAGGCGCCTTCACCTACTCCCCGACGCCGGTCACCTCGGGCATCGGCATCGGCATGTCGGCCATGGG
CTCGGCCACGCGCTACCACACCTACCTGCCGCCGCCCTACCCCGGTTCGTCGCAAGCGCAGGGAGGCCCGTTCCAAGCCAGCTCGCCCTCCTAC
CACCTGTACTACGGCGCCTCGGCCGGCTCCTACCAGTTCTCCATGGTGGGCGGGGAGCGCTCGCCGCCGCGCATCCTGCCGCCCTGCACCAACG
CCTCCACCGGCTCCGCGCTGCTCAACCCCAGCCTCCCGAACCAGAGCGACGTGGTGGAGGCCGAGGGCAGACACAGAAACTCCCCCACCAACAT
GGGGGGGGCCTCCTGTTCGCGACAAGCCCGCCGGGATCCCGGGCCCTGGGCCCGGACACCGTCCTGGGGCCGAGGGCGCCCGACGGACAGGATC
TCGCTGTAG

TABLE 19

MOUSE NOMENCLATURE
ICSGNM I12ra
Celera mCGSSG9
HUMAN NOMENCLATURE
HGNC IL2RA
Celera hCG23482
MOUSE SEQUENCE—GENOMIC
TAATGATTAATAATAATAATAATAATAATAATAATAATAAGAAGAAGAAGAAGAAGAAGAAGAATGAGAGACCTTGGAACACTCAGCCCCTTGT
TGTGCATGGAACCCAGTAGAAGAAGTGGGAAGAATGTGAGAGCCAGAGGGGATGGAGGCCATCAAAAAAACACAATCTTCTAAATCAACATCAG
CAAAGCACCTATGAAGAGTGAAGCAGGACTGGAGAGATGGCTCAGTGGTTAAAAGCACTGACTGTTCTTGCAAAGGTCCTGAGTTCAAGTCTCA
GCAACCACATGGTGGCTCACAACCATCTGTAACAAGATATGATGCCTTTCTCGTGTGTCTAAAGACAACTACTTACACATAATCAATCAATCAA
TCAATCAATCAATCTTTGGGCTGGAGCAACAGGGGCCGGAGTGACCAGAGGTCCTGAGTTCGGTTCCCGGCAACCACATGAAGGCTCACAACTG
TCTGTACAGCTACAGTGTACTCACATACACAAAATAAATAAATAAATCTTTAGAAAAGAGAGTGAAGCAACATCCACAGGGCCTGCATGGGTCT
ACACCAGGTCCTTTGCATGTATATTATGGCTTCCAGTTTAGGGTTTTTAAAGGATTCCAAAATGTGTGAACAAGGTGGACTTGATTCTTGTACC
TTCTCTTGGGCTCTTTTCCTTCTGTCTGTTTTCTCTAAATCCAATGTCTTAGTTTTTTGTATACTTAAGGTTATAGTTAGAGTAGCATACCATA
GCATGTCCTTCACAAGCCTATTTCTTTTCTAATGAGAGACACAAAAGGAGTAGATCTGGGAGGGAAGGGATAGGAAGGGGACTGGGAGGAGTAG
AGGGAGGGAAACCATAATCAGGGTATATTATATGATGGAAAAATCTATTTTCAATAAAACGAAATAACTTTAAAGAAAGATAATTAGGGAGTAA
ATATGATCAGGTGAATATATGAATGTCTGAAAATGTCATAATGAAATACATGACTGTACACTAACTTTTTAAAAAAAGGAGTGATCTTGGGACT
ACAGACTCTTCAAAGATAACCAAAAAAAAAAAAAAAAAAAGAGAGAGCAGGCTGTTGAGCCGAATAGTGATTGAACACGAAGTGTACCGCAAAA
AGTGGGGAAGCCATGTTGAAGAGGGTAGTCTAAGGCAGATTTCTTTGGGAAGATTTGCCTGGAATTAATAGATGAGACTGGAGGAGGTGTGAGG
GCTGGCTCAGTAGCTAGGCACTTGCTTAGCATGTGCCAGGCCCTGCTTTAAATACCAGCACCGGAAAAAAAAAACATAAAGGATTGCAGAATGT
TTGAGTCAATCATCTCAGTCGGAAAGCTATAGTTANNNNNNNNNNNNNNNNNNNNCTATAGTTACATAGACCTCTAGACAAAACGTGACACAGA
CATGACTTCACCTTCCTCCCCAAACCCCACAGACTACTGCTTCAGAACCTCACAATCTTTGCTTCCACCAGTCCCTACAGGTCACCAGTGCTGG
ATCGAGAGATTGAGCGATATACTATTGTCCACCCTTGAGCTAGAACCACATCAGAGGAGCTCAGGGAGCCTTTCCTGATACCTACGTGCCAAAG
ACACAATAGAAAGTGGGATCATCGATTGGGGGGTGCATCAGCCAATGCAGTACCTGCAGTAAAAGCATGAAGACCTAGCTTTGGTGCCCAACCC
TGAGGGACCGTGTCTCAATCTCTGATGAAGAGTGATTGAGGAAGAACCTCCAACAACAACTTCCTCTGGTCCCCACACACCTGCAAACACCAGC
ACTCATGTGGGTGCGCGCGTGCGCACACACACACACACATGCACACGTATGCACACACACATACACTCATAGCCACACATACATACATATACAT
ACAAACACACACATACACAAATGCAGACACACACAAAAACAGTCATGTATACATACAAACACACATACACACATACAGGCATGTGTGTGCACAC
ACATGTACATTCATACACACATAAACACACATGCACATGTGCACACACAGCCACACATGCATATACACAGTCATGTGCGCACACACACACACAC
ATACACACACACACACACACACACACACACACACAAGGAATCTGTCTACAAGCTAGCATAAGCTGCAAATGATACCAAAGCTGCTGTTTGGTTT
TCAGTGTAGGGACTGACTTTGTGAATCTCACAAGCTGTTAGTCTCCCAGTACTTTATTAGGGGAGTAACTCACCAGTCAGATGGCCCCCTCTGG
AGCTCAGCATTTGCACAGAGGACAGCTCCTTTGCGCATACTGGATTCTGGTTTTCCACAGGACCCTCTCTAGTAAAACCTACTGGAAATTACAA
GCTGAGAAATTCAGCCTTGTGCCACAATAACAGGATGCACCTGTAGATTTCACAGAATTAGCTGCTGTCTTCTCTTAACGCCAATTTGAGAGAA
AGAAGCCTGTTTGTCTCCTGCCAAACAGGGCAGAATCTCAAGTATTCCCATATATCTGCAAATGATGGAAGTTATCAACCACAGTGACACTGTC
TTTCTAGCGGTCACTTGACAAAGACACGGACTTGCTTGCCTCTCTTTTCCACCATGCTACCCCCTTGTTGGCTCCTTTGGGACTTACCCATTTG
GAATGTGATATAGTTAATAGCGACCTGTCTGGCCCTGCCTCCCCATCTCCATTTGAGAGCACCAATCCTTTTCAATTCATTTTTGATTACACAG
TCTGGCACAGGGCCCACTTTCTAGATCCTTAACTAGTATTTTATGCAATTGAATTCTAGAAAAGTACATGAACTGTGTAAAGGTGACATCATGA
GCACTTAAATAGACATCCAAACCTATGTCCACGCAATAAAGACTCCGTGCCCCATAGGCTGACAACCACCTCTAACTAACCCAGAAATGCAGTA
AATATATAAATGATGAGTTCCCTGAGCTACTAGCCATCCTCCAAGAAAACAAAGGCCCCTTTCCTCCTCAGCTCTCTCCTTTTCGACTTTCCCT
TCCTCACCTCCATTTTCTTCCCCTCTCTTTCCACCTACCCTTTCCAGTCCTCTCTCCTACCCCTCCTCTCTCTATTCTTCCTTCCTTTCTTCTC
TCCCCTTTCTCTCCCCTTCTCTTCTTTCCACTCTTCTTTCTTCCTTCTCTTCTCTGGTTTTCTTCTCCTCTCTGCTTTTCCTCGCTCTCCCTCC
ACACCATGCCCCTCTCCTCCTCTCTCTTTTCCTCTTCTAATTCTGAGACCACAAATGAGCACATCCCTCTTCAGACAGGCAAGTGAAAAGACAG
AGTGACGGAGCTTTTATGTGGTGTCACTCGGGTAGGACAGGCAAGTCTGATAGTGCCAAGGATCCAGTGAGCAACAGCTTCCCTTTTTAAGGGC
TGGTTTCTTTTTCTCCTTGAGTAGATTATGGGCACAGACAAGGCCTTGACTTCTCTACTTCAGAGCCCAGTGTAAGAGCTTGTGTAGTTGCAGG
TGCTCAGTCAACATCAGCTCATGGCCTCCTTAACAGAAATGAAACACTTTGACCTGAGTTTCAGTTCTTCCTCTCTGTAGACTTCTCTCTAGAC
TTCTCTGTAGACTTCTCAAGACATTGATAGACAATGAATAGACAGGGGTAAAGGGGAGAAGAGAAGAGAGGGGCGAGAGAGAGACAGAGACAGA
GAGACAGAGACACTGAGAGAGACAGAGACAGAGAGACACACACAGAGAGAGATCGTGATACTCTGAACTGCACACCTCCCTCTTATGCCTGGTA
CACAAAGAAAGCCGGGCTCAGAATTCTAACACACCCTCAAACCCGATTCAACTCCAGAATGTATAAGAAAAAGTAATTTCATTTAAAAAGAACA
TCTTTGAGGTGTTGAAAAAAAAGAGTTACTGCCACAAAAGGAACTATAAACAATTACTGTCAAATGATTTTTAGGATTATTTATTTATTTAATT
GAGACAGGATTTCTTGGTGTAGCCTTGGCTGCCCTGAAACTCATTCTGTAGACCAGCCTAGCACTGAACTCAGAGATCCACCTGCCTCTGCCCC
TGGTATTACAGGCATGCGTCACCACCAACACCCCCCCCCCCCATTTTTAAGGATTTCTTTCCCTTTTTTCTTTTTAAAGATTTATTTATTGTTA
TATGTAAGTACACTGTAGCTGTCTCCAGACATGCCAGAAGAGGGAGTCAGATCTCATGACAGGTGGTTGTGAGCCACCATGTGGTTTATGGGAT
TTGAACTCAGGACCTTTGGAAGAGCACTCAGTGCTCTTACCTGCTGAGCCATTTTGCCACCCCCCTTTTTAGGATTTCTTTTACTCTCTTGATT
GTGCCCCCAAAATTGCCCCAGTCTTTCTATTCCATCTTGATACCACATTTCTTTTTCTTTTTCTCTTATTAACTCACTTTCTATGAGTATTTAT
GTGTGTGGAGTGCACACATGTGTACATGGTTGCACATGTGTGTGTATCTGAGCTCCTTGGTGTGTGCACACATACACATGCATATTTGACGTCG
GGGTGTCTTCCTTGGTCACATTCTACTTCATCTTTTTGAGGCAAGTCTTTTGCTGAACCCAGAGTTCCACTGATACAGAGTCTAGCTAGCCTGC
TCATTCTGGGGATCCCTTTCCCATCCGCCAAGTGTTGCCGTCAAAAGCAGCCACTGTGCTGCGCTTTCCCATTGGTTTTGGAGGTTTGTAGTCC
AGTCTTCACACCTGTGTGGCCAGTGCTTTATCCACTGACCCATCTCCCAGTCCTTCAGCATCTTTTCTAGAGCACCAATTTTCTCTCTCTTTGC
ATCTCTAGTTGCTGAAGCCTTACCTGCCAGGTCAAACATGTCAGAGACGCAAAGAGACAGAGAAGTTCTGGGAATCAGGGCTTCACACTTGCTA
ACCCAATACACTGTCTTTGAACTATGCCCCATCCTGAACTATGTCTTAACAACTGAAAGTAATGGTAAAATAATCCAACTGATACCTGTCTTAA
AAGATAAATATTTATTTTTACTTACTTATTTATTATTCTATGATCATTGGTGTTTTGCCTGTAAGTATGTCTGTGTGAGTGTGCCAGAACTCCT
GGAACTGAAAGTATAGATATTTGTGAGCTGCTATGTAGGTGCTGGAGATTGAACCTTGGTCCTTTGGAAGAGCAGCTAGTACTTTTCATCACTA
AGTCATTTCTCCAGACCAATTATCTTTGTTTTTTCTATGTATGAGTCTTTTGCTGTGTGTATGTATACAAATGCATCGTGTGTGTGTGTGTGTG
TATGTGTGTGTATGTGTGTGTCTGTATGTGTGTGTGTGTATGTGTGTNNNNNNNNNHNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNTTAGCACCAGACTGCATCATAACACACTGCTCGT
ATGTACTGTGATACTGCACTTCATAAGTGACTACAAACATTATCTTTCAATTCTTTTAAACAAAGAATACGCCGGGCGGTGTGGCGCATGCCTT
TAATCCCAGCACTTGGGAGGCAGAAGCAGGTGGATTTCTGAGTTCGAGGCCAGCCTGGTCTACAAAGTAAGTGCCAGGACAGCCAGGACTATAC
AGAGAAACCCTGTCTTGAAAAACCAAAAAAAACCAAAAAAACAAAAAAACAAAACAAAAAAAAAACCAAACTTTGATTCTGGAGGTGTATCACT
TAGCAACGGAGCATGTACCCCATTGATGGGACATGTATCAGTACATGTAAGACCCTGGGCTCCAACCCCAGCCACAAACTAAACAACAACAAAA
AGCCCAACTTCTTGAAAATGTTAAAAAAAAATCCTTAGTCTTTGATTTTCTTTTTTAAATGAGACATTGGGCCTTCCAGAACCAGAGAAAATAA
GAGGAATAGAAGACAAAACCTCAGTAATAGGATTTAAAAACAAACAGATGACTAGTTCTACAAAATACCTTTGTTAAAACAGTTTACATGACTA
TTTTAAACTGACCTAACAAAAAAGTAATGGAAATCTAATCTTGGCATGGTAGCCTATGCCTTTAGTCCCAGCACTTGAGAGGCAGAGGGAATAG
ATCTCTGTGAGTTCAAGGTCAGCTTCGTTTGTCTACATAAGAAGTTTCAGGGCAACCAAGGCTTCATAGAGAGACTATGTATAATATGCTAGGC
CCTCCCCCATCAATTACTAGTTAAGAAAATGCCCTACAGCCAGATCTTAGGGAGGCATTTTCTCAGTTGAGGTTCCCCTCTTTCAGATAACTCC
AGCTTGTGTAACATTGACATAAAACTAGCCACCACATTGTTTCATCCTGTTTCAACATGTCCTGTCGCTCAGATTCTATCCTTCAAAGTCTTCC
TCGCCATCAATCCAACTGACATCTCTACCCTCACATTCCTTTGGCCTCCCAGTCAACTGTACTTCTAGTTAGCTGTTGAGCACAGATTATACCT
GAAGGACCTACCTAAAAAGGCTACATTCACCCTTCAAGGACAGCAACTTATGTCTTTCCTGAACATAGTCATCCATAACGTTCTCTTTGCTAAG
CGTCTCCCGCTCTTCTTCATCATGATCACTGGGCAACTACCACCTTCTACTGTTAGAAAGAGCAAACGCTCACCTCTAACCTAAGAGGAGGCAA
TCTGGGTCACATAGTTGATGCATGATATGATGTGCAGTTTCTTCTGAGAAGTACCAGACATTTCTGATAAGAGAGTTGAGCAACTTCCTCATAT
GTGAGACTCAGCTTTATACAGTTGACCAACCAACCACCAGTCCTGAAGGAACATTAGTGACCCTTAGACAAATGTTCCTTAAATTAATCTATTG
GGGTCATCTAGCCTACCTTTTTCCTTCCTTAAAAGTCTTTGCCAGGAAACAAGTATTTGAAATAGACTTAGTGGAAACTACCTATTTCCAGCAG
GTTTGAAACAAAACTTGGACATGTGCTCACAAGCATTTGTCTGAAATGGCTTGAAATGTATCCTTGTTGTATATTCACATTCACATATCTCAAT
GCATCATGGTTGAACACCCATATGTGTTATGAAATCAGTGCTTATTTCAAAAATATATTCCATGTAGCCTGTCTCCACACCCCACACTGTGATA
CCTTTATCCTAAGATCCTCAGACTCTTATGAGTTCAAAGTGCCTGTGACACTCCTGTAACACTCCTGTCTATAAATATACTGATTATATGCTGG
CGTGCATTTTTCCCTCAAGGGAGGAAATTAAGATTTCACCAGATCTTATTTGCAAAACAAAACAGTCAGCAACTAGGCTAGAAGTAAAAAGTTA
TGTCAATAGTGGGTGAAAAGACAGCTTGGTGACACTATGAGAGAAGGCAAAGGGTTCTTTCTGAAAGGCCTCCTGGGAGAAGCTAGACTTAAAA
TCTTCCATTGCAGCTCTAAACACATCTGGACAATAGTCTGTTTTCTGCATTTGTGAATCCCACACCCATGGAACTATGAATCGTGCATCAGAGT
TATTTAAAACCACCGTGCATGGAGTGAACCAATACCGAGGTGTTTGCTTATCATTTTCCTTTGAGCACACAGCACAGCCTTGAACTCAGTGACA
CTCCTAAGAGGGCTCTAGGGTCAGGCCAACTTAGATCAGATGCTAGTCTTTAGCTAAAGATGCCCTTCCACCCCCGTTGCACGACCTTGCTTCT
CAGTCTTTGTTGAGTCTTCTGGGGGAGAATCCCCCTAGAGGACTCAGTTTACAAAACCCTAAGTGAGACCACTGCCAAGAAGTGCTTGCTCACC
CCTCCTCCCGCGGCAGGGAATCCCCCTTTCCTTGTACAGGCAAAACACAAAAAAGGACTCATAAGTGAAGCCTGATCCTTCTCACCAAACACTG
CCCACACCTCCTAGTAATTGAACTTGAAAAAAAAAACTGGTTTGAAAAATTACCGCAAACCATATTGTCATAAAAAAAAAAAAAAACACTTCCT
ATATGAGATCACAGAACAGAGTAGGCACAAGTTCCTGCTGAGCAGATCAGCCTAATGCTTAAATAGAACAACTCCTGGCTGTCATTGACATTGT
CTAAAAGCCAAGATGACAGACTGAGACCCCTGAGCCCTTCTTCTGGCATTCTCCCAGGAAGATCCAGTAAAGGGGTTGACCCAATATACTGCAG
AGAATTTCATCCAGTTCCCTCCTCCATCCTGATCCCATGTGCCAGGAAGATGGAGCCACGCTTGCTGATGTTGGGGTTTCTCTCATTAACCATA
GTACCCAGTTGTCGGGCAGGTAAGAGACCAGGAACTGCCCTGGGAACCTTGGGAAGACTAACAGAATCTTGGTAAGCCTACATCTGCTCAGCTA
TACAACCAGAGGGAACAATGAGATGGAAAACTTTGAAGAGGGAGTTGAATCATGCGTTCTAACTCTTGTCCAGGGTCCACTAGGACCTCTTTTT
GTGCCTCAGTGGTCCCTAATCTATCAACCTTTCTTAAAATAGCAGGAAGCAATTTCTCATTCATAGTCCTTTTTAGGTTTCATTCCCAGGGATG
GTTTGGCTTTGCTGTCTTGGAAGTTTCTTGTAAATATCATTTTATGGCAATTTCAGAAAACATAAATATATTCATTTTTCAGAGCATGACTTTC
CTCTGCTTCTCTGGGTGTAACGTCTGTGTGTTTTACATAAATATGAATTCCATCATCATAGATTTTGTCAGGTATAGTTTTAAGAAATAAAAAC
CATCACCAACTCAGACATCAAACAACTCTTCTTAATTGAGGATGAATTCCCTAATACCATTTTTTCTTGCTAATCCAGAAAGAGAAGTGAGGTT
TGTGGAAATACATATATTTCAAGTCTATTAGATAGATTATAGATAGATAGATAGATAGATAGATAGATAGATAGATACATAGATATGGGACTGT
CTCACTATGTAGTCTTGGGGTGCTTGGAATTTGCCATGTAAACTAGTTTCGCCTCAAACTCATAAAGATCTACCTGCTTCTGTGCATAACCCCA
CACTAGGCTGAAAATATATTTTTTGAAAGCCTACAGTTTCATATTCTTAATTTCTAGGAATTTATTCTTAAATGACCCCAGAAAAATGTATTAA
GAAGAGTAAAATAAATAACATATACACGAAGCTTGATTGCTGACTGGGGGCAAGTGATCACATTGTGTTGGGTCACTTGGGAGCATCTTGGCTT
TGGAGTTAAGATGTTTATGATTTTAGGAAGCTTTTATTGTGTGATTTCTCCCCTAAAAGTATCTATCCCTCAATGGTGTCTTTGTCAGGTTAGT
TCAAGTCAGAACAACCAAGTCTTTACAGTTATGTGTATTCTGTCTCTGAGGAAAGGCTTTCAAGTAAACTTCTGTTATCAGCTGTGCTTGATTT
ATATAAAGCTCATTTACTTGGTTTTGCAAAACATCTGTCCACTACTTCTTATGCATGTGTATATGGGGAAATGACAGCATTACAGGTTGGAACA
TGCCTGGAGCTACAGGGGTGTGGGGCTGTCAGAGAAGACTTCATGAAGATAGCGTGTCCATTGAGCCCTGGAGACTGGTAAATTTCCATAAGGA
AAGATTTTAGATGAGAAGGCACATGGATCAGGAAATAGAAGAGTCTGAGGAGTCAGTATATGTATTCTCTGAATATGGGGCTATTGGATAAAGA
ACAGAAAGATAGGAATGGGGCCAAAGGCATTAAAGCTCTGGACAGGAAGCTCAATCCTTTTCTTTTCCTGTGTTCTTGGGGATAGAAATCCAAG
GCTTTGAGCACACTAAGCAAGCACTCTAACGCTGAGCTGCATTCCTAGCCCACAACTTAAAATACTTTAGTTGTATTTGGTAGGCAACAGAAAA
CTACTTGAAAAATGTCAGGGAATGCCATTTATAGAGTACACTTGATAAATCAGATGTATACATTTTATTACAACTTCTAATCACTTTACAGTGT
GGGTGTCATGAATCAGTAGAAGTAACCCCAAAATCAGAATATCAGTGTTGCAAGGCTCACATCACTTTTGTATTTTCTATTGGAGACAAGACCA
GAGGTGATTCTGGCTGATAATAGAAAGAGATCAGGTGCAAGGAGTTATGTCAAGAGGCAACTTGGTTCCCAGTGGGAAACAGAAAGAAATGGCC
AGAGGTCAGCAAGTAGCTCAGAAACTAACCCAGGGCAAGAGATGATCTTATTTCAACTTTGTTTATCGTCCAGGAAGAATTTAGTTTAAGCAAT
TAATGGGGCTGGAGAGATGGCTCATTATTGGTTTAGAGCACTCATTACTCTTGTGATGCGTTGACAACCCAGGACCCAAGATGGTGGCTCAGAA
CCATCTGTAACTCCAGTTCCAGGTGATCTGACAACTTCTTCTGGCCTCCATAGGCACCGTACACATGGCAAATAGGCATAGATGTGGGTCAAAT
GCATATACACGTAAAAATAAATAAATATTAAACTTTAAGAAACGGTAGAAAAATAAACAAATCTTTCTATTCCTTGGGGGCAAGAGGTCTTTAA
TGAAATAAGACAGACTGTCAACAGATTCAATTTCTAGCTGATTCATAAGGCAGATTTTCATTTTGAAAATTTCCAAAGCCACCATTTAAAATTG
TGATGTGGTACAAGGGATGTATTAGTCAACCCGGGTGTTTTTAGTCTCTCTATCACTGGGCCTTGGGATGTGGTTAAAAGAGAATTCTCCCTGA
TGTTGCCAACACAGCAGCAGCTAACTGAAGCCACCTTTCTTAGAACTGTGGAAACTCTGAGCTGTCTCTTGATCATTTTTCATTCTAGGAATTC
TGTAATCAAATCCTTATCCCTCTAGAGTCAGAAGACAGAAGGTATGGAAACATGTTAAGCTGGTACGCATGTCTAGTTATCCCTTCTGGTAAAG
GCTACTGCCCACACTAGAAGTTCCGGTCTTCAGCTCCGTTATGCTTCCCAGTGCCCCGTCAGTGTCTTCACAGTGTTAGAGCTATTGTGACTTT
GTTCATCATCCAAAATAGACCTTGGAGACATGTGGTTTTGCTAAGCACTGGGAATACTTAATAAAGCAAACATTACATTTGTCCCTAAGGGTCT
AAGATAGCCTGATATGGAACTTATGCCTGTGCTTGATTTAGCTTGGAACATTGAAAAAACAGTAATTTATTTCATCCACTTACATGTCATCTGG
CCATGCTCTCAAACAACAGCTTCCGAATGCTCAACACCTCCATCACTGGGTCCCTCCCAAACGCAGGGATTTTTGTCTTCATGTATGCATATGT
GTATTTTTGTGGCTATGTGTGTAGATGAATGTGTGTGGAGGCCAAAGGTCAACTTAGGTATTAATCCTCAGGTGCCATTCACATGAGTTGGTTT
TCTTATTATTTGGGTTTTTTATTATTGTTGTCTAGAGATGGAGTTTCTTGCTGGTCTGGGACTACATGGTTACCCTAAGCTGGTTGGTCAAGAA
TCTGCCTACTTCTGCTTTCTTAGGGCTGATACTAAAGGTATGCACTGCTGTAATATATACATACATACACACACACACATATATATACATTATA
TATACATATACAATATATATACATTAAATGTATCAGTCTTTTGCCTGCATGTTTATATGCACACCACATGCCTGCCTGGTGCCCTCAGAGGTCA
AAAGAGGGCATTGGCTCACCTTGGACTGGAACTATAGAGAGTTTTCAACTCCCATGTGGGTGGTGGGAAGCAAACCTGGGTCCTCTGCCAAAGC
AGGTGCTCTTAACCTCTGAGCTATCTTTCCAGCTGCATAATCATTTATGTGAGCACCAGGGATCAGAACAGAGGTCCTCATATTTATGAGGTAA
GCACTTTATCTACTCAGCCATCTCCCCACCCCTGAACATCTCTCTCTCTCTCTCTCTCTCTCTCTCTCTCTCTCTCTCTCTCTCTCTCTCTCTC
TCAAAAACTTTGAGAATTCTAGATATTTTTGTGTAATTTCTTATGGAAACCATTCCTGTGGTAGCACTGAGTGCCATGAGGATAGGCTGCACCC
TGACATGTTCATGTCAACTCTGCTTTCCTTACTGTCCAATGATGGCCGGGCATTTTACAGATAGAGGTTGAATTCTCTTAAGAAATAGTGTGCC
ACCAGTGGTCAGTGAGAAATAGAGCTAGGAGGCAAATGAAATTTTAAAGATGTATAGTATACAACAAGGCACCTAATGATGTTAGAAAAACCAA
AGGCAAGACCTAGCAAGTCACAGCTTTCAGCAAACTTCTCTCTGCAACAGGAGAAGGTTGTTGCAAAGATCTTGAAATCCCCTTACAATTCCAT
CATTATACATCAAGAATTCTGGGGGGAAAAAGACCTACTCTCTCCTCTCCTCTATCTCTCTCTCTCTCTCTCTCTCTCTCTCTCTCTCTCTGTC
TCACAAACACACACACACACACACACACACTCACTCACAAGTCTTTTTTATCCTGGGGTACTTTTGGAAACAAGAAAAGTTCCCAACCAAAGAT
ATGCCCACTACCATGTGGATTGAAAGGTCACCGGGTATCAGCAGACCCAAGCCTGAGAGCTGGGTTCCAGGTTATTTCCCCACTCTCCAGAGTG
CAACGCTCTGCACTCCACCATCACTACTAATTCTGTACTGCTCGAGTTGTGGCTGTAGCGTAAAAACACCACTGTCTTTCGGTGAAGTCTTCTC
CCGTTTGATGCCTTGCTATGCGGATTTCTTCCTTCAAACTGTGTTCATGTCTCGAAAAAGGAGTGGGAATTTATCTCTTTCTTCTCTTTGTTTT
GAGCCATTCCCTCTGGGGGAGGGAGGGAAGGGAGAGTCTTTATTCAATGCATTACTTCAAAAAGTTATAGATCCCAGACCTGAGATGCCTCTGT
GGTTAAGAGTGCATACTACTTTTGCAGTTTGGTTCCTAGTACCCATGCTGGGAGACTTACCCTGCCTATTTCAATCCAATGACCTCTTCTGTCC
TGCATGTATGGATAGACACATACATAAATAAGACCTTGCTGTCCTGGAAATCAACCTGTAGATTAGAACACCCACCACCTCAGAGCTCCACCTG
TCTCTGTCTTCTGAGTGCTGGGCATGCACCACTTCTGCCCAGTGATCCCAGGCCTCTTGATGACTTGATTACATCTTCTGTATGCCTTTATTCT
GATTCTCAGAAGTGATATCTCTTTATCCATGCAGCCACCCATTCATGCATTAGGATTATAGAGGTGGATAAAGGTACAGTCCTTGTTCTCCAAC
ATGAAGACTAAACATCTGCTGATTGATTCTGATTAGGAAGACTGCTAAGGGAAAACTTGCATCTGCCAATGAGTCAACATTCTGTTCCCTTTCC
AACCCTCTGCCACATTTTCAAGGACATCACCCATTTGCAGGGAAAACTACAACTAAGTGATCTCTGAACATCTTCGGTGTTTTCTACTCTAATG
TTGCTCACATCTTTTTTGGCTAGTTGATTTTATGGTCATTCTAGATTTTGCCTTTATCAACAAGACAACGCAGGTCTCACCCGGAGCTGTCTGT
GGAATCATGTTTCCCAACCCCAGCTTCAGTTACAACATGCATCTGGAATGGCTGAGACTTCTTCAGACCAGTGAAGTCAGACTCTCAGGGGAGA
AGAACCAGACCTACCAATGACATTATTTTTATGTCCCCCCAAACAATGAAGATGCAGTGAGCAATGAGATCCGTCTAATATTGTAGAGAAGTAA
TATAATGGAATAATCCTACAGCCGGGCATGGTGTACATCGCTGTGACCCTTCCTTGCACTTGGGTGATTGGTGCAGGAAGATTTTGAGTTTGAG
GCCAGCACAGAGCTCCACAGTGAAACTATTTTAAAAAGAACGAACAAGAAGAGAAGGAAGAGGGGAAGGGAGAAAGGGAGGGAAGGGAAGAAGA
GAAACGCAAATGGTTTTTCATGCTGGTTAATTTCATTGCTTCATTTTTATTCTTTGAAAATAGAGGAGAACTTCTCAAACTTTACAAGACTCCT
GGGGATTTTGTTATAAAGCATTCTGTTCGGAGAGGGATTCGGTACCTTGAAATTCTGTTCAAACAGGCTCCAACACGAGATGTGTGTTGTCAGT
GCATGGGCCCAGTTCTCAGCAGCAGGCTATCATGTAACAACAACAACAACAAAAAAATGCACACTATGTAACCACACAGAGGTTTCAACTAAAA
TGGGAGACAACTTCAGAGAAGATTAAATATTGATCAAAGGTTGCACAGTCTATATAACACAGCCTATAAATATTATAAGTCGTCAGAATAAAGA
GAGCAGCCTACTGCCTGGGTAATTAAAGATGATGCTTTGAGAAGGGCTTTTGGAGAACCTAGCTCTGAATGATGCTTAAGAATAACAGAGCATT
AGCCGGGCAATGGTGGTGTACACCTTTAATCCCAGTACTTACCACGCAGAGGCAGGCTGATTTCTGAGTTCGAGGCCAGCCTGGTTTACAGAGT
AAGTTCCAGGACAGTCAGGGCTACACAGAGAAGCCCTGTCTCAAGACAACAAAACAACAACAACAAAACAAAACAAAACAAAACAACAGAAAAT
GTTAGAAGACAGTATTCTGTGATCATTCTCCCAAACCTCTACACATCCGCCATTATCCGTTTGCAGAGATGTGTGTCTGAACAAAATGATTTTC
AAGTGCACAGTGTTTCTCTGATAAAAATTAATACTTTCCCCACATAAAAAAAAAAGCTGAACATCGTGGCACATGTTTGTAATCGCATTGCTGG
GAAAACAGAGATCGGTAGATCGCTGGGGCTTACTGGTCTACCAGCCAAATCAGTGAGCTCTAGGTTGTTGGGAAACCATATCCCAAAAGAAAAG
ATAGATGGTATACACACTACACACACACACATATAGTAGTAGTAGTAGTAGTAGTAGTAGTAATAATAATAATAATAATAATAATAATAATAAT
AATAATGATAATGATAATGATAATAATAATAATGATTTTGCTCAGGCATAAGACTTTGCATGTTCCCCATCCTACTTAAATCCTCCAGTGCCCA
TAGGCATTAGTTATCTCTAGTAGATTTCTTTGACATGATGAATTACTGAGTAGCCTTGCCAGTACCACATGACACCCTGGATATAGTCAAAGAA
TTTTTTAGTTTTCTTCTACCTCTACATCTGTGTTTTTAACTATGATGCAGTCCTGTTAACCTCATACCACAAGGTCAGAATCCCATGGCTTCTA
ACACGACAGCCTCCTTTTACCCTGCCAGTTCACTAGGCTTCAACCAGCAACTCTCAAGGTAGCACCAGACATCCTACATGCAGGAGGCTGAGGC
TCACATGACTGGACTACCAACAGTAAACGGTTTCACCCAAAAGAAGCACTGTTTTTAAATTTTTATTTATTTATTTATTTATTTATTTATTTAT
TGGTTTAAATTTTTTCTTTTCTTATATGTATGGGTGTATGGATATTTGACCTGCATGTATATATGTGTACTCTCGGCATGCTTGGTGCCAAGGA
GGCCAGAATAAGGTATTGGATCCCCTGAAACTGGAATTATGGAAAGTTGTCAGCCACTATGTGGGTCCTGGGACTTGAACTCAGGTCCTCTGCA
AGAGCAGCAAGTGTTCTTAAGTTGTGAGGCTTCTCTCCAGTTCCAGAAGAAACATTTCAAACAGTGATGATCATAGCGAGAGCTTATGCTCACT
GTCTACATGCCATCTCTTAGTATTGTGTTAAATCAGTGTCTCTTGGTTCAGTTTTCAAAATAAGGTAGTTGTGTTGGCCATTGTGGTCTATGCC
TTTATGTTCAACACTTGGGAAACAAACGCAGCAGCTCTCTACACAGTGCCAGTCTCCTCTACACAGAGCAGTGTTCTCAACCTAACACTGCAGC
CCTCTAATGGAGTTCCTCACACTGTGGTGACTCCCCAACCATAAAATTATTTTTGTTCCTACCTCATAGCTGTAATTTTGCTACTTTTATGAAT
TTTAATTTAAATATCTTTTTTTCCCAATTGTCTTAGCAACCCACAGGTTGAGAATTGCTGACACAGAGAGTTCCAAGCCAGTTAGGGCCACATA
GTGAGCTCTGGAAATAAAACAACATAAAAAGCAGTTCTTATTATCTTCCCTTTGCAGTTGACCAATGGAGTCTTTGGTCAGTTAAATCAGTAGC
TGGTGTAACACAACTAGGAAGTCTCTAGAATCTGAGTTTACAGAATCTCTACCCACCATCCTGAGCCATGACCACCTTACACAGCAGCTCCACA
GTCTACCTGAATGGCCACCTGTTGAATCTCTTGTCTTTACTTTTGTGCATGTGTGTGTGTGTGTATACATATGCATGTATATACACATGTGTGT
GGAGGTCAGAGCTTCATTCACACGACATCTTCTTTTATCCCTTGACCTTATCTTTTTGAGACACTCTCTCACTGAACCTGGAGCTCACCAATTC
AGCTAGACCACCTGCTCAAAAGCTGCAGTGGTCTTGTCTTCCCCAGCCCTCTGCTGGGGCTACAAACATACATCATGGTGCTGGCATTTCTACC
TGGAGGGTAGGGAATCCAAGCCTGAGTCCTCTTGCTCTCCCAGACAGTTACACCTCAGCATACCCTCAGCCTCTTTTTTTCCCCCTGAATGTAT
GTAGCTCAGGGTGGCTTAGAAATTCATGAAGTGATTAAGGCTAATCTCAAACTCCTGATCACTCCACCTTACTTACACGTCTGGAGTTCACTCT
GGTAGGACCTCCTCAGGACCCTGCTAGTCTTAGATCCTGTATTCTCTGAATCCTTCTGTTACCTTCAAAGAGCAGCCTGGCGTGTTTTCCTTGC
ATCTGATAGCATTTCAAAGAAGTCACAGCAGCAGAGTCTGGGCTAGCAACCGGCAAAACAGAGGAAATTCTGAGCACCTTTGGCCATCAGTCAC
CACACCTTCTGAGCACCTTTGGCCATGAGTCACCACACCTTGTTCAGGAATTGCCATCTCTGCCCACAGAATCGCCCACTGTTTTCTGTGTGGG
ACCAAAATAGATACCTCCTTCATAAACTTTGTGTGTGTGTGTGTGTGTGTGTGTGTTCTCTCTCTCTCTCTCTCTCTCTCTCTCTCTCTCTCTC
TCTCTTGGTAATAAAAAACAAAAGACAAACATGGGTATGTGCTTCCAAGGGCAGACTAAGAAGCCACTTACAGGTGAGTGTGACAGCTGAGAGG
AACCGTGGCAAGGAACACCCTACCCTGGTGGCAGAGCTTCTCGATATTTGTATGTCTGCTGCTTTGCTTCTCAGTTCATTCATGATCTATTGAG
CAACTTCACGCAAGGTTAGAGAGGCAGAGATGAATAAGACCCAACTCTTGGCCTCCAGGAGCTTCTTGCTTATGCGGAGAGAATACCCAAGCTA
AGGGCTGAGATTTAAGTAAGAGCACAGAGCTGGTGGACACACACTCCACTGCCCACTCCTCCTGCAAGACCGACACACCATAAGCTAGGAACTG
CTTCCCATTATATGTACATGCAGAAGAGAGAAGGGCTCACGCTGTTTTGTCAGTGTGAGTGACTAGAATTAGAATCCAGCACTTTCCATTTTGA
GTTTAGAACAATCTGCAGAGGTTTTGTGCTTGTTTGGCTCAGCAATACCTTTCAGCGACATCTCAGGTAGGACGTACTGGGAGTCCTTCAGTAA
CAGACTGTTTCCACAGGCTCAGGAGTCTCTGAGGCACCATCTATAGGCCAACTCTCACATGTCTGAGTACATTTGAAGATCTCCCAGGTCCTCA
ACATCTATGACCTCTTCAAGGAAGAAAGGAAAAGGGGGAGGGAGGGCAGGAGGGAAGGNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNCTAAGGATTGAGCCCAGGGTTCAATTCACACTAAACAAAACACTTTACCTCTGAGCTGGGTCCCC
AGTTCTTACTATACTGTATAAAGCCGAAAAGAAAAGCAGTGCTTTAAAGATGTATTAATAAACTATAAGAATAATACATAATAAATTCATATGT
AAAAAACCACAATGTATATATGTAAATGAGAGCAGCCCTAAAACCAAACAGGCCTGTGTCCCCACCGAAAAGGTCACTTTATCATGTTGTGGGG
AACCGCAAAAGAAATGTTTCTAAAAGTTTCTTTTGAAGTCCCCGACATCTATGCTGACGTCAGCCTCTTCCTGCCCAGCGCTCTGAAAGTTTCC
AGGGAGTTTTGGGGCCAGTGACTTGACACAGGGTTGTATGTTTGACACTTGCTGACTGTGGGCACGTTGTGGTGGCTTTCCCTGCCTGCCAAGC
CCCACTCAGCTGAGCATCAGCATGGTATGTGCTTCCTGTCCCCGTGCTAGTGACACCTCTCTGTTCAGACCTTGATTTAGAACAAAAAAGAACC
TTTGTGAAGAGAAAACTCCTTATTTAAAGGAAAGTAAACCGAACTTTTGAGAGTCTTTTTAGAATAAAAGGGCGACTCCTCAGGAGTCCCGCTG
GTACCCCTGAGGGGAGGTGAGCCTTGAGGGCTCGCTGCTAGGTTTCCCTGTCTGGTCCCAGACATCACTTTCCCTTTCTAACTGTCTCCTCAGA
GGCCGCTTCTGGAGGTAATTTTTAGCTGCCAATGCTGAGGAACTATTGGCCAGAGCATGCCACCCTGCTTCTTAGAGAATACAACTGCCACCAT
TGAAAACCTAGGCTGTCTAACCAGTGAGCTCAGGGCGTCCGCCTGTCTCCACCTCCCTTTTCCTCCTGTGCTGGGATTTCCATGTGCACAGCCA
TATAGAGTTTATAGAGTGCTGGGGATGTGAACTCAGGTTCTCATGCTTCTGTCGCAAACCCACGAAACCATCTCCCATCCTTAGTTTTATATAT
TTCACTATACATTCATATTATAATATACATTATATATTATATAATATATATTATATAAAACATATGTAATATATATTATAATATATAATACCAT
GGTATTGGCAGTGCTTAGAATAGGTGCCCCAGATTTTCGTAACTTTTTCTTTGTTTTCTGTTTCTCTTCTAATCTTTTTTTTTTAAGTTGGTTA
TTTTATTTTTTTACATTTCAAATGTTATTCCCCTTCCTGGTTTCCTCTCCGCAGCCCCCACAATCCCATCCCCCTCCTCCCTGCTTCTAGGAGG
GTGGCTACCCCACCCACCCACCCACTCCCACCTCACTGCCCTGGTATTCCCCTTCACTGGGCATCAAGCCTTTACTGGACCAAAGGCCTCCCCT
CCCATTGATGCCAGATAAGGCCATCCTCTGCTACATATGCAGCTGGAGTCATGGGTCCCTCCATGTATGCTCTTTGGTTGATGGTTTAGTCCCT
AAGAACTTTGGGAGGTCTGGTTGGTTGATCTTGTTGTTTTTCCTATCGAGTTGCAAACCCCTTCACCTCCTACAGTCCTTGCCCTAACTTTTCC
CCTGGGGCCCCTGGGCTCAGTCCAATGGTTGGCTGCAACTTAAACAGCTGTCCCACACCATGGTTACTTCTCCAGTCTTTAATTGCATTTGTTT
AACAATTTATTTTAACAATTTATTTTCTTATGTAATATATGTGTGTGTGTGTATACATCTATATATATATGAGAGAAGAGGGCATCAGAAAACA
GAGGGTATCTGATCCCTTTACAGGTTGTGAGCCACCATGTGGTCGATGGGAATTGATTTCAGGACCTCTCTGGAAGAGCAGCCAGCGCTCTTAA
CAGTTGGGCCGTCTCTCCAGCTTGTATTTGTTTGTTTACTTATGTATTCATTTACTTATAGACAGGGTCCAGCTATGTATCCTGAACTGACTTG
CAGTGTAGCTAGGCTGGCCTCAAACTCATGGTAATCACCTCTGTCAGCCTTCTGAGTTCTGGGACTACACATGCAGGGGAAATCCCACAGATAT
CCATTTGAAACAAGCCCTCACGATTACAACTTGCTTCTCAGAAGGCTCTGTCTTTTTTCAGAAGACTGTGGGCCGGATTTACCAATTTAGCAGT
GCTCCGCTAAAAATATGGCCCCCTCCCAAGAAATCCGTTTCTAAGATTTCAGGCGAAAACTGTCTTTAGTTTTCAGAGAAAGAGGAGGAATATT
TCTTCCCAGACTTTTCTCTCTCTCTCTTTCTTTTTTAATCTGATAACACTTTAATTTAAATCAAGTTGACATTCTGTGGTAAGTTTGTGGTTAA
CTCTTTCAAGTGCTGAAGCTTGTTTTCTGAGGTGACTGGAGAGTTAGAAAAAGACAGGCAGCCCAACCACTGTACTCTGTAAGCATTACAGGCT
CCTGGATGTGACAATCTACAGGCCGCTCTGTGAGACAGAGGGCAGTTCTCAGCTCCTTGCCTGGGTTTCTCTTTCTAAGAGCAAATTCCTAGTT
TCTGTAGACCTACTGTTTGTTGTTTGTGTAACTACTGCCAAGAAGGCACAGGGTTTTCTCCAAAATGGTCTTACTAGTGAGTCACACATTCCTG
CCTTATGGAAACCCAGGCTTTGACGAAGCCTGAGAGAACTGGGCCTCAGAAACGTGTGTCAGATGACAACAGGACTCACAGACACAAAAGGCTA
ACCACCGTTCTGCAAATATTGTTATTTCACTGTTTCCCCTTTAATTTTAACAAACTTTTCTTTAATAATCACACACACACACACACACACACAC
ACACACACACACATTTTTGACAGGGTCTCACTGTGAAGCCCTGGTTGTCATGGAACTATCCTGGAACTCATTATAGAGACCAGGCTGGGCTAGA
ACTCAGAGATCCACCTGCCTCTGCCTCCCGAGTGCTGTGATTAATGGAATGTGCCACCATGTCTGGCTTTCATGAGTATTCTTTCAATCATCTT
TGGATTCATTTCCTGCTATTGGCATCGCCATATAATTTTCCACCTTTAAACAGAGTCATATCAAGAGATTTTCCACTAGTGAACCATCCATCAC
TGCTGAGATAAATTCTAATTGATCAGAATATTAGTTTTTGTCATACCTGGCTAGAACCAAGTACAAATATTGTAATCGATATTCTGTTGTCTGT
TTTTATAAGTGAGATGGTCTTACGGCCATTTTCTCTGCAGTTCATCCCAAGTTTAATAGGAACACTAACATAACTCTGTAAAAGGAATTAGAAT
CATTGAAGTGTCTGTCCTCGTCTGAATGAGAATAGCTGCCATAGGCTCATAGATTTGAATATTTAGTCCCCAGTGAGTGGAATTTCCAGTTTGG
GAAGGATTAAGAGGGCTGACCTTTATTCAGGGTTTTGAAAGACTCATGTGATATTCCAGGGTTGTTTTCTCTCTCTCTCTCTCTCTCTCTCTCT
CTCTCTCTCTCTCTCTCTCTCTCTCTCTCTCTCTCTTTCTCTCTCTCTCTCATCGCTGTGGACAGAGATCTGAGCTGTAAGCTGTTCCTTTACT
CTACCATCATGGATTCAAACTCCCTGAAACCATATGCCAAATTAAATGTTTTCTTTGTAAGTCCTGGTACCCTATCTCAGCAATGGAAAAGTTA
ACTAAGATGCCCTCTGAAACAGTTTGTATTAAGCATCTGCTACCTATTTCATATATTTGTTCACAAGTTGATTTTTTCAAATGATTGGTTTATT
AGGTATAATTCTCCTCTTTCTTCAGCTTCTTTCTCTCTTTCTAAGACAAAATGTCGCCATGTACCTCAGGCTGGGCTTGAACTCACAATCCTGC
TCATTTTTGAGTTTTTCGTGGATCAGTAGTTACACCTCCCTTTTCTTCAAACTGATGCTCTGTTGTTCTTGCTCCTTCTAGTCCTGACTATTTT
CAGAATCTACTTAAATGAATAGTTTTTAATTTGGAGGACATTCTCTGCTGGTTTTTTTTTTTTCATCTTGGTCAATCTTTCATTTACAGTTTTG
ATCTCCTTTCTTTAACATTATTCCATGGTTTTTTAACTTCCTCCAGTAAATATTAAATGGGATAATGCCTGTTCTTTGAACACGTATTTTCCTT
TGAGGTTGTAGACATCCCTGGAGAGGCTGTCCTGGCTCAGTACTGCAGGGTTTCTGGTTATCATTTTTGTTGCTATTCGGTGCAGAATATTTTA
CAGAATGAGTTCTGAGTTTCCCTTTGATCCATGGATTTGTTTGGAAGTTTGAGGTTTCTTTTTTAAAATGTGTGAAGTTTTATTGTTGTTTGTG
GTTTCGCTCTCTGTTTTTAACTTTTAATTTCATTGCGCCATGATTAGAAAATGAATCTGTGTGATGCTTATTCTTTGACACTTGTGGAAACCAC
CTCTGGTGCCTAGTAGCAGCTCAGTTCTCTTTACCACTGTCCCATCCGTGATAAAGAGAGTGTACATTGCCTATGTATTGGTCACACAACTACA
TAAATATGCACAAATCAAGCTTACTGACAACTAACAGACCTTCTTATAGAGTAACCTTTTCTTATTAGTGGTCCTTGCAACATCAGTTTCTTTT
GTAGTGAGTTCTGGGCTTAATTCTAGTTGGGTTACTTTTTGTGGTTTCCCTTGGCTTCCTGAGCTGTATTTGGTAGCATCTGATTGGCTGACCT
GCTTTTACTATAGGATTCCCATGAGTGTGGTCTCTTTGATAGTTCAGGAGTTGTCTTCTAGACACTGCAGTATGTGAAATGTATTATCTTTGGT
TATTGCCGTAGAGTCTGTGTTATGTAACTTGGAAGAGTGTAGGGTCATTTACAGCAGTGCCTCCCTTGTGTGGAGACAGCACAGTGTGTTACCT
CTTTCTGTTGTTACATCCAGTGTTGAGTCTTCACCCTTGATCCTAACCCCACTCACTCCCCTGCATCTTGGGTCTTTTGTCTCCTTCATTATTC
ATGTTGAGATTTTGTCTAAGTTTGAGGAGGACTAAGTAATGAATGGAATATGGTATAAGTTCTCTCATCTGCGATGGAAGTATATCTTACCTCC
TACCTTGTGTGTTCACAGTGTTGGCCTTGATCTCCAGAATCAACACGAATGTCAATGGTTCAGAAGCAAATATGTTCTACAACATTTATTTGCG
TTTATACAATAAAACCCTACTCAAGTAACATCTACAACACCAATGACCGTAAGAAAGTGAGGACCTAAAATCAAACACACAGAAGAGATATATA
ATAATGAAAGTTACTTATGCTTATAATATTTCTAATTATACTTATAATAATTATAAGTATACTTATACTTAATTATACTTATGTTAATTCTACT
TTCATTATTTAATTATTTAATTATTTAATTATTTAACTCTACTCTCTCTATAAAGGCCCGAGTTAGATTATTAATAAATACAACTAGATAAAGG
AGTGAGAAAAGACTAATGATGGGACACAAGGCAAACTTAACAGGCAAAGTTAAGCAAGCTGAAGCTTCCCTCTTGCGTAGGTGATGAAGATGCC
TCTTTCCCCAGCTTCTGTTTTCAGAGTTCACTGTGCTTGTGTGTGTGTGTGTGTGTGTGTGTGTGTGTGTGTGTGTATGTGTTATGGCATGTGT
TTATGTTTGTACATGTAGTGTGTATTTATGTGGTGTGTGTGTGTGTGTGCGTGCATGTGTGTGTGATATATGTGGTATGTGTGGTGTATGTGGT
GTATCTGATGTGTGTGGTGTGTGTGTGTGTGTCTGTGTGTTGCGGGTGGGCCTTGTCTTCTAGTCAAACAGGTCAAACCAAACACGACAGCACC
TGTTTGGTGCTGTTTGGTGCCGACAGACACTCGGTTCCAGATGCCTTGACAAAGTCACTGCAAGGCTATATTCACACCCCTGAGCTACCCTAGC
AAATCCCAGGACTCTGTGAGGTAGGAGTCACAAGGATGCTATTCTTGGTATCTTTTGTGATGCAGCAGGAGGGGTTGCTTTAGCAGGCCCATGG
TGAGGCATCCCCCACCTCTGGTATCAGCCGTAGGACAGCTCTAGTATAAAATGGAGTTTATTTGCGACATGGGAGTAGAGGCAGAGGAAGGAAG
GGGACAGAGAACAACAGAAAGAGAGAAGAGAGGAGGAGAGAGAAAAAGGGAAGAGGCCTTCCAGGAACATGCTGAGGGAGTGGGAGACAGAGAG
AGAGAGGGCAGAGAGAGAGAGACAGAACAGGGAGAGAGAGGAGGAGAAGGAGCAACAGGGAGGGAGAAGGGGGAGGAGGAGGAGGAACAGGAAG
GAGAGAGAGGCCAAACAATCCTTTTTATAGCAAGTCAGGCTAGTACCTGGCTCTTCCTAGGTAACTGTCGGGCAGAGCCTACAAGAAAGGCTAA
CAATTCTCACTGGCCATTCTTGTCCCTGTGCCTGCAGGAGTGAAAAATCCCCATGTCGTTGGCTAATCCTCCAACTGTCTCAGAACCTCAGAGC
AGGAGACCATGACCAAGCCAAAGCATTTAGTGCTGAGGTGCAAGTGAGGCGTCATCACTGACTGACTCATTGCCTCCTCCAAATCCCCGACAGC
TGCCCTTCCCCTCCTTATTAATAAGCGGCATGTAGCTAGGCATAATGCTGGAGAAGGAGCTGAGAGGTTGACGTCTTCATCTGCAGCCACCAGA
AAGAGATGTGTGTCACACTGGTCATAGTTTGGGCATATGTGACCTCAAAGCCAACCTTCAAAATAACACACTTCCTCCAGCAAGGCCACACCTC
CTAATAGTTCCACTCCCTATGGGTCTTTGGGGGGCCAATTACATTCAAACTACCATAGTGCCTTGGTCAGGGTTTGTATTCCCACACAAAATAT
CATGACCAAGAGGCAAATTGGGGAAGAAGAGTTTATTCAGCTTGCACTTCCATACTGTTCTTCATCTCCAAGGAAAAGTCAGGACTGGAACTCA
AGCAGGTCACAAAGCAGGAGCTGATGCAGAGGCCATGGAGCCATGTTACTTACTGGCTTGCTTCCCCTCCCTTGCTCAGCCTGCTCTCTTATAG
AACCAAGACTACCAGCCCAGAGATGGCACCACCCACAATGGGCCCACCCACCCTTGATCACTAATTGAGAAAATGCTTTACAGCTGGATCTCAT
GGAGGCATTTCCTCAAGGGAGCCTCCTTTCTCTGTGATAACTCCAGTTTCTGTCAAGTTGGCACAAAAAACCAGCCACTACACACAGCTACCCT
TAATATCCAGATAGTTTCTCACTAGTGTGACTCCTGTGTGCTTGCCTCCTGTGTGACAATTAAAAAGGACCATCACGCTAACCTACTATATGCA
AGGGAGTGCAGTAAAGAAACAGCTAACATGATTTCTCCCATGGTGTGGTTAAAGTTTTTACTGTAGAGATGAGAAAAAGCAGACAGGAAAAGAA
GCAGACTGATCATGCCCAGGACAAGGGAAGCAGGAGAGAATAGTAGTGCTCGGGACGGACCAAGACATAGACGACAGAGCCTAGTAAGACAGGG
TAAGTGAATAGAGAGAAAGAGCAGGAATAGTGGAGAATAGTGGGTTATAAGGAGGCTGCATAGGCAGGGAGAGGGAACAGCCTCGGCACTGTCA
CGGTGGCTTTGAAACATATAACATTGTTGAAATAATTAAGTAATAAGTTAATCCCAGCACCGGTTACTTCCCACCTCAATGGTTTATGTTCCCG
AACAAAACATATACACACAGTCTTTATATTTTAATATGCCTTAAACAGCACAATAGCTGGGTCACTGCCAAACCTCCATGAGGTTAATCCTGAA
TTATTACTTACTAATTCTGTGTTCTATCTTGGCTGCTCTAGACCAAGTTGGGCAGCCCCCTAGGCCATGCTCTCAGTTTCTTCACAAGATCGCT
ATGCCTCTCTGTTCTCCATTCTTCCCCCATCTCTCCTCTCCTCCACACTCCCCCTACGTGGTGGATCATCTCTTTCTCCCTCCTCAGGGTCCCA
AGCCTGGTAATCTTTAAATCCTGCTTCTGTCTGTCCTCTTCAGCCATTGGTCATTGGCATCTTAATTTACCAATCAGAATCAACTATATGTAAC
TTTATATATCAGTAAACAACAAAACAAAACAAAGCAAGATTGGGAAGTAATCTAAGCAGCCATTAAGACTTGTGGAGGATTTGTAGATAATAAA
GCAACAAGAGATTATTTCAAGGGGGGGTGCTCATCTTCCCGCCCTTTCCAGCAAGAGACCCTCCTTGTAGTCTACTGAACACAGCAATGCATTT
GTGATGATTAATCTCCTTGTTGACTTGAGGAGGGTTAGAGTCACCATGAAAGCAAATCTCAGGCATATCTGTGAGGGACTTTTCAGATTAGGTT
AGTAAGTATGAAGACTGACCCTAAAGTGTGTGGTGTCATTTCCAGTAGCTGTAGTCCTAGTTGGCATTAAAAGGAGAAAGCTAGCATCCATGAA
AGGATGGATCTCTAGCATCCATCTCTCTCTGATTCCTGATTATGGATCCGCCATGGTCACTGGCTTCAAACTCCTGCTGCTGGGACTTCCCCCC
ATGATGGACTGCACCTGAGCCAAGATAAATTCTTTCTTTCTTTCTTTCTTTCTTTCTTTCTTTCTTTCTTTCTTTCTTTCTTTCTTTCTTTCTT
CTTTTCTTTCTTTCTTTCTTTCTTTCTTTCTTTCTTTCTTTCTTTCTTTCTTTCTTTCTTTCTTCCTTCCTTCCTTCCTTCCTTCCTTCCTTCC
TTCGCTTTTGTTGGGTATTCTGTTACAGCAAAGAAGAAGTTGGTAGGCTGTGTTTCACAACGCCTATGCTAGGATGGCTTGTTTTGTTTGATCT
GAACACCCGTCAAGCCAGGGTAAACTTGAACCTGGCTATGACGCAGGGGCCAGGATTCTCCCCAATGGGCACCAATGTCAAAAGCCCAGAAGCA
CAAATCCATATGGCGAAAGGCAATGTCTTAGAGACTTTCGGATGCTGTTCTCATCATATCGCTGATTGACGGTTCAAGGAAGCTGCCACATGCT
GTTTGATTGGCATTTGGATGCTTAACATAAAGCAATAACAAAGTCAACCTTACTTGCCAAGAACTTAGCATATGTTAGACAAAAACTTCCTGGG
TCAAAAGAGAGCAAAGAGAGGGTTCCTATCCGGTGGTTAATCACAAAGCAGAAGGGCACATTGGGTTTCACTTCCGGTTGGTTTGTTCAGATGG
TATTATGGAGGCAGTTGATAGGGTAGAGAGAAGTCGTACAGAGAGAAATGGTGAGATGCTGTTGCCACACAGGTGGACAGTTGCTGAGTTGTAT
GGGAGTCTTGAACGTTAGTGGAGAGGGAGTATGAATGCCAGAGTATTGTGGCTCTTGGCAACTGAGCGGACATCAAGAATCAGAAACATGTGGA
ATTCGCTCAGATGGTGACAGTCACTAACAGAAGCAGGTCAGAAAGGGAACAGGGTGGTTTGTATGCTTATTTCTAGCTTTTTGTTTCAAGAGCA
TTGGAGATTCACACTCTCTCCTCATTCTCTTTGTTCTGTATTACACAGTCAGTTTTCTATTTAAAATAATTTCACCAAGGTAAAAGGCAATTTT
TAAAAAAAGGATAAACATAAAGGCAGCCATTAGACTCCTGGCTTTCAGTTCCCCCCACTATGAAAATGCGGTTTGGGTAGCCTGACCTCTTGGG
ACCTCAGTCTCTAACTGTATGAAGCCAGTTGAGAATATCTTCCCTTTGTGGCTGCACAGAGCATTGGAGGTAATATACACAAAGTGGAGTTCAG
AAATTAGGTCTGTTATTACATATCTTAAAAATCCAAGCAGAGGGACCTGGGTGCTCTAACTTCACAGATAATGATCTGAACTGAACCACAACAT
CAATAAACACTGCCTGGTACCCAGATGGCTTAGACAGATGAGACATCTCCCTGAAAGTGACAGCTGTCAGATGGGGAGTGGGGGCAACCTGTCT
CCTGGAGGTTACATCTCTCTGACATGGAAGGAACATTTCTGAACTTGGAGAACTCAGGGTTTTCCACAATTCACAGAAAGGGCGCAGAAAGATC
AGTTTCTTTTTTATGAGTCTATCACTCTCAGAGCATTTCATCATGTTAAACAATATTCATCATGTTCTACTGAATCGTCTTTCACATACCCAAA
CACACACACACACACACACACACACACACACACACACACACACTTTCTGTAAGGCCCAACCATTCATTTGGTCTGGAGACAACCCTGACCTTGG
CTTTCTTATCTGATCTGATGAGGAAGAGGATGGCTCTCAGGCAAAAGCCACACTGTTCATCAGGTGACTCACACTCTTTTTTCTTTTTTCTTTT
TTCTTTTTTTTTTCGAGACAGGCTTTCTCTGTGTAGCCCTGGCTGTCCTGGAACTCACTTTGTAGACCAGGCTGGCCTCGAACTCAGAAATCCG
ACTGCCTCTGCCTCCCGACTGCTGGCATTAAAGGCGTGGGCCACCACGCCCGGCGACTCACACTCTTGTTACACTCATGAGTCCCTTATATAGT
TCAGTCAATACCTGTCCCGCAGACACCATGCTTGCTGAGTGTTTTCTAACCTCCCTTCTCACTCCCAGTATATGGAAAACCAGGAGGCAGCAGG
GAGAAGAAGCCCTTACTCAGCGTTGGCTGAGCTACAGACAAAACATTTCCTTAACCACTCCTTTCTCAAACTCTGTCCACTTTCTTAGGGAGCC
CTCCTTTGGAGTCATTTACCTAGGGCACAGGAGTCCCTATCGTTCTCATCTTAAATATCATAGATAAACCAACGCTACTGTCTCCCAGCCTACC
TTTCCTCCGGCTACCGACTAGATCCCGAGTAAAAATACAAAGCACTCTGAAGGCACGTTCCAAAGCACGAAGGGTCTGCTCAGTGTGCGCTGGA
AGACAAGAGTAGCTCGGCAGGCAGTACAGACAGAAAGTAGCGTCCAGCAGCCAGAAGTTCCCCCTCAACCTTAACTATCTGCTGTGTCACAGTC
GACAGAAAGCATCACCACCGCTAGAGGGCTCACTTCTCCAGCCCAGTCTGTCTCTTCAGGAGGTTTGAATGCCTGCCACTGGGAGCTCTCTGGT
CGTCCTCCATTTAGCGGGCGCTGTCTTATTGATAAAACAACCACTTGCCTCCCGAGAACGCTTCAACTGTCAATATTGCTGTTTAATTCAAGGG
GCACAGAAGAATTCAAGGCACAATTAATTCTAGTTTCATTTTGTTTCTAAAAATAAAACTGTTAAAAAAAATCAAGGGCAACCTTTTCTATCTG
AGGAGTGGAGGCGAAGCCATAAAACCATGTAAGGAGGATGGCAGTTAGCGTAAGAAGTACGGAGCCATGTCTGTCCTCCTTGATCTTGTTACTA
CATATAGAGGTCTCTTCCTGGGAGCAGAAGGGTTTGGCGAGAAAAGCACAAATGACCTCATCCCACAGCAAGGCTCTACCCCTAAAGGTTCTGC
GATCATCCCAAACAGCGCCCCATGCTGCCGACCAAGTGTTCAAACATAGGTCCCTATGGACTGACATCCACATTCAAACCACAACAAGCACCTT
TCCCTACCTGCCCTGTGCTGCAGCCCACAGGGAGGAGGCTTTGTAATTCCCAGAGTATTCGCTGAAGGAGTCACAAGTTCCCAGAACGTCTTGT
ATTCCATTTTTCCTGGAGACACTAGATCCCCAGATAATTCCTGCATTATGGTCTTATTTCAGGGAGAGTGACTGGGGAGAGATTTGGCAAGCAT
GGGACTACCCCTCATACAAGATGGGCAACATACTCCATCCTGGAAGAGTTTGGCGTTAACTTTCCCAGTGTAGGTCTCTGGTCTGAACCCTTGT
ATTGGGTTGATTAACAACTGCTGGCCGCTTCTTTCGCCCTGTGCCTACATGAAAGGCAAAACCACATCCAAGTACCTCGGAGTCTGAGATAGGA
GTCTCCCAGGAGCCCCATATCCCAAGAATTAAGGTGGAAAAAGGCTGGGAGGTGAATTTGGGAGAAGTGGTTTGCGACTATTCAGGAGGTAAAA
GTAGAGAGGGAAGACGCCAGTCACCTGAAGTCATTTTAGACAATAAATTAGGATTCCTTTTTTAAGTTGTGTACATGGAATTCTCCTTCATGAC
CTCCCAGAGAAGCTTGTAAACAAATTATTTTTACTCATTTTTGGCTGTTGCGCTGATGTTTCTTTTATGACACCAGAGCAACAAGAGCTAAGAA
TATCCCCAATTTACTCTTAAGGGATTAAAAAGAATAAAATCTCTAGGGCTGCCTTTCCCATGGGCTCTTCTGCCTTGGAGTCTTTGAATAAGTT
TTGGAACACATTTGGCAAGGCCTTTCACGGAGAGAGAATCATAAGCAATTCTGAGAAAGGTTCTTTTAGAAATGGAAAATCTGTGAGAGTACAA
AATTCTACAACTGTCTCCGTGACCCCACATGACCTCCTTGGACACTGGACTAGTTCCGGATGACTGTGGAAATTTCCATGGCAAACCCTTGTCT
AGAAAAACATCACCGAACTCTGAGGAATGCTTAGGGGTGCAATGTCATCCCAAAGAAAACTTATCTGACCTTTCTTAAAGTTATGCATGTGAAA
GCTTTCTTAGCTTCTGCTTTGGTACCAGATTCCAAATATGATCAAGATGAATCCCTCACTATTTGCACTGACGAGTAGTTACTCAAGAATTGCT
AAATTAATTGGTTTTGTTTCAGTGAAATTAGCTATAGACGACTTATTCTTAAAAGGTTTTATTTATTATTTTAATGTATTTAATGTGCCTATCT
GTGCCTGTGTGAGTGCATGCACACTTGTGTGCATAGAAGCACATGCCTGAGTGTTATTAATGTCCATAAGATATTCCGGTGTGCCATTCTGCCT
ATTCCTTTGAGGCAGAGTCTCTCCCTGAACCTGAGGCTCAGACTCTCTCAGCTAGTCTGGAACCTACACTATTCGAGCTGTCTTCCTGTTCCTG
TCTGCACTTCCCCTCCGGGCTAGTGTTCCAGGCTTTTGCAGGAAGCCCCCATCTTCTGCAGGGGCCCTGGAATCCAAAATCTCGTCCTTGTGAT
TGTGGGGCCAAGAGCAACAGCGGATCTGTCTTTCCAGACCCCAAACATGCAGGCTTTATTTTACTAATCACTATTGTGGGGGTGGTCATTGGGG
TGTGTGTGTGTGTGATGTAAGAAAGACCTTATAGGGAAGCTATATGAATAGGTCTGATGCAACTTGATGAGTTTTGACAACTGTGCGCAAGAAT
ATGAAGAACACTCGGTTGTTTTTTTTTTTTTGCTGTCTCCTTCGAGTTGACGCTGCCTACTACAAGAAACCACTGGCCTAATTTCTCTCTGTTT
AAGTTCTCTACCCCCTCTCCCTCTCTCTCCCTCTCTCCCTCTGCCCCCCCATTTACATCTAAGTGAATCATGTAAGTGAAGTCATGTAAGTAAG
TATGACTAGCTTCTTTAGTTTCTTTTTTTTTCGACATTTCTCTATGTTGTGTGGCAGGAATTCGTTCTTTTGAATGCTTTGGTGCATTTTTGAA
TTTATACTCTATTCTCACAATGATAGGTACATATGTTACTGGGTTGGTTTTGTTGCTTTAGAGATAAGGTCTGGAGTAGCCCAGGCTGGTATCA
AACTCATAACTCTCCTGCTCCAGCTTCCGGAGATCTGGGATTATAAGTATTTCCCACCATGTCCCAACTTGCGTTCGTTATTAATTAGAAGTCT
GTGTCAAATTCTCTCTTGGTGCACATTATTTCACTACTCTGAGACAAATACTTCAGGGTGGAATTGCTACACAGGTCTATATTTAACAGTATCT
GAAATATATTCTTTAAATTATTTTAGATTTATGATGTGCTCATCAGTATTTTGCCCACATAAATATATATGCACCATACTCATGCCGGCTATTT
GCAGAGGACAGAAGACAGCATCACGTCCCCCTGGAAATGGAGTTACAGAGACTCTTGAACCACAGTGTGAGTTCTAGAAACCAAATCTTCATCC
TCTACAAGAATGCCACCTCTTAATTCCTGAGCCATCTCTCCTGTTCCATTTTAAATACCTTAAACCATTGGTTCAAAACCTTCCTGATGCTGCG
ACCCTTTAATACAGTTTCTCATACTACGGTGAGCCCCCACCCATAAAATTAGTTCGTTGCTACTTCATAACCATAATTTTGCTACTGTTATGAA
TCGGTATGTAACTATTTTTCGAGCTAGAGGTTTCCCAAATGGCTCACAACCCACAGGTTGAGAACTGCTACCATAAACAGTTTGCCCAAGTAAT
TTTATCTTGTGCTCTCAACACTATATAAAATAATTCCAGCAGCTCAGATTCCCCTTCACATTTGGTGCAGATTTCTGTCTATTTTTTTTTTTTT
TCTATTTCACCCATCCTAACGGGCAAATTCATGGTGGTTTTCAATTTGTGGCTTTTTTTTTCCCTTGAAAAAGTTATTTATTATTTGTGTGAAG
TGCATAAGCACACACGTGTGCAGGTCAGGGAGCAACTTTCAGGAGTGGGTTTTCTCCTTCATCCATGGGTTCTGTTCAGTTAACTTGGGCCATC
GGGTGTGGGGGTCAAGCACACCTAGCTTGCCTCTCTCTCTCTCCCCATCTCCCTCTCTCCCTNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNACATTTTTTCAG
GTGCTTCTCACCCCTTCCGTATTCCTCAGTTGACAATTCTTTGTTTAGCTCTGCACCCCATTTTTAATGGGGTTACTTGAATTTCTGGAGTCCA
GCTTTTTGAGCTCTTTGTATATATTGGATATTAGTCCCCTATCACATTTACGGTTGGTAAAAATCCTTTCCCAATCTGTTGGTGTCCTTTTTGT
CTTAATGACAGTATCTTTTGCCTTACTGAACCTTTGCAATTTTATCACGTCCCATTTGTTGATTCTTAATCTAACAGTACAAGCCATTGCTGTT
CTGTTTAGGAATTTTTCCCCTGTGCCCATATCTTTGAGGCTTTTCCCCACTTTCTCCTCTATAAGTTTCAGTGTCTCTGGTTTTATGTGCAGTT
TTTTGATCCACTTAGACTTGAGCTTTGTACAAGGAGATAAGAATGAACCAATTTGCATGATTCTACATGATAACTGCCAGTTGTGAAAATGCTG
TCTTTTTTTCCACTGGATGGTTTTAGCTCCCTTGTCAAAGATCAACTCACCATACGTCTGTGGCTTCATTTCTGAGTCTTCAATTCTATTCCGT
TGATCTACCTGTCTGTTGCTCTACCAGTACCTTGCAGTTTTTATCACAATTGCTCTGTAGTACAGTTTGAAGTCGGGCATGATGATTCCACCAG
AGGTTCTTTTATTGTTGAGAATAGTTTTTGGTATCCTAGGTTTTTGTTGTTGTTGTTGTTATTCCAGATGAATTTGCAAATTGCCCTTTCTAAC
TCAGTGAAGAATTGAGTTGGAATTTTGATGTGGATTGCATTCAATCTCTACATTGCTTTTGGCACGATAGCCATTTTGACTATATTAATCCTGC
CAATCCATGAGCATGGGAGATCTTTCCATCTTCTGAGATCTTCTTCAATTTCTTTCTGCAGAGACTTGAAGTTCTTATCATACAGATCTTTCAC
TTCCTTAGTTAGGGTCACACCAAGATATTTTATATTATTTGTGAAGGGTGTTGTCTCCCTAATTTCTTTCTCATCCTGTTTATCCTTTGTGTAG
AGAAAGGCCATTTGTTTTACTTAATTTTATATGCACCTACTGCACTGAAGCTGTTTATCAGGTTTAGGAATTCTCTGGTGGAATTTTTGGGGTT
CACTTATATATACTATCATATCATCTGCAAATAGTGATATTTTGACTTCTTCCTTTCCAATTTGTATCCCCTTGATCTCCTTTTGTTGTCGAAT
TGCTCTGGCTAGGACTTCAAGTACAATGTTGAATAGGTAGGGAGAAACGTGTATTTTCTTAACAGCTTGGTGACTGCCACTTCAGTGAGTGTGT
GTCTTCAATAATCACAATATTTTCTTTGGAAACATGACTATTCGAGTGTTTGTTCATCTATCGATTCAGTTGCCCTTTACTGTTGCTATGTAGG
TTTTTGTGTATTCTGGAAACAAATATTGTCATATATACATTATAAATGGTTTTCTCACAGCCTGCCACATCTCCTCCATTTTCTCATGGTCTTT
GGATTAGCTGATACTTTTGTAATTTTTTTTTCTTTTATAAAATCTTTAACCTGGAGCAGGGCATGGGAGGGTCCACTCACACCCTCTGATGCTG
GGGTTACAGGTGCTTGTGAGCTGCTTGGTTCAGGTGGCTGGGAAGCGAACATTGGTCCTCTGCAAAGGTAGCAAGCATTCTCAGCAACTGAGCC
ATCTCTCCAGCCCCCTTTTCCTTTTATATTCAGTGCATGCTCTGTCCTAACAAATCTTCACCTATCCACAACTTCTAAAGCTGTCTGCCTTTAT
TTCCTTTTAGAAGTTTTACAGTTTTAATTCTATGATTGCATGTGATACACACACACACACACACACACACACACACACACACACACACACGTTC
TTATTATGGAAGAGAAATAAGTTTTCTCTGTGGAAGGAAGCTCTTTTCCCTTTTCACATTTATCCACACGACTCAAGAGTATTTGTTGAGAACG
TTGTCCTCTTTTCATGAATTGCTTTGGTAAATCGGCGCGGGGGCAGTAATTAAATGTTCCTCAGTTTCTAGAATGTATATTCTGTCGCTGTACT
TGTTTTTCTCCCAGTTGCTTGCTGTCTTGATGGCTAGCTTTATAGTAACAGATGTTATGAGTCTCCTATCATACACTTTATCTTACAGTTATCT
TAGATACTTCCAAGGAAGTTTGAAAACAATTTTCCAATTTTACTAATATGTACATCAAACCAGTGAATTGAAGGTAAACTGACATGTCTTTTAA
AAATCAGCCCTCCAATTTATAAACAGTGTATCTTTCTCTGTTTATTTCACTTAAAAATTTTTTCTCAGCAGAAATTCTTTTGCAATTTTCAAGT
CTGTTCTTGTAGGTGTGGGTGAATTTATTCTTGAGTATTTTATGATTCTGATGTGTAATTACAATTAACTTTAGATCTTAATTAATTCATTCAG
TTTAAATATATAACAAGTGATTTGAGATAGCCACACCTACCTACTTACATACGCCATTATGTAGAATTCTCGGTACTTTCATGCAAAGAGGCAT
TTTTAGCTGTGTTAAGTGCCCTTTCCCACAAAACTACTTTCTGATAGTAACATAGCCCATATCAGCTTTCCTATGCTTATCTCTGCATATCTTT
TTCTATTCTTTCACTCAATTCATTTTGGTTTTTATATTTTAGCATTTTCTTTGTGAGATAGTATGCAGTTGTTGTGGTCTGAAAGTTTCTATTT
TAGAAAAAATTGTTCATTTATGTTTACTATAATCACTAGTAGTGGGTTGACGTTGATGTCTGCCTTCTTGCTCTTTCTTCGTGCTTCTTCATTT
CACCTAAACAGATCTGTTTAATACATATTGGAATGTTCTGTTTAATTTATTCTGTTATCTTTTAATGTGTGCTCCTGGTAGTATTTCATTATTG
GGTGCCTTCCCTGGAATATTTACTATGCATTCTTAATTTTCTATGTTCTATTTAGTTAGCATTGTACCATTTCAGACATGCTAGAAAAATTTAA
AAAGCTAAACGCCCATCAACCTCAGCTTTCTTCCCCAACTTGTCACGTTTTACATTTAAGTAGGTACTAAGATCTGTCCTGTAGGGACCAGCAC
GGTGGCTCAGTGGCTAAGAATGCATGCACACTGCTCTTCCAGAAGATCTGAGTTTAGTTCCAGGCACTCACACTGTACAGCTCACAATCACCTG
TTTCTGACCCCCTCTTCTCCTCTCTACGACAACTCCACTCACCCCCTCCCCTAACACACACACTTAAATCAAAAATAAATAAAAGACCTACCAT
ACAGTGCTATTTTTTTTTTATTTTAGACATCTCTTTCAAGAAAAAACTGGAATTTTCTCATGAATATATACTTACTAATGCCCTTCATTATTCC
CAGTAGATTTCTATTTTTGACTAATATAATTTCCCTGGGTCTTGACATTTTGTCCTTAGCTTTCTTTTCACTTTTAATATGCACTATAAGTGTG
CTAGTGGGGTTTTGTGAACTTTCTTTTACCTGAAAATGTGCTAACCCCATCCTCATTTTTGAAGGGTGTCACGTCTAATCAGGATTGAGCTGAA
AGCCTGTCTGTGTGTCTGTTTCGAGGCTGGCTCTGTTTCCCTGTTATTTTCTGGCTCCCTTTCCTCTGTCGTGAAGTCACTTCTCATCCATCAC
TGTTCCCACTAATGTGTGTGGCCTTTCCCTCTGGCTCCTTTAAGACATTCTCTTTAGATTTTTGGCAGCTTGGCATTAGATGCATAACAATTTG
CTTTTAAAAGTCTACTCTGGTGTCATGAACATCTTGGATCTGTAAGTTAACATCTGTCTTCAAGAGTCAGAAATGAGCTGGCAAGATGGGTAAG
TGTGTACAGGTGTTTCCTATTGGTGGCCCTCCCACATTTCATCCTTCCCAAACAGTTCCACCAAGCAGGACCAAGCATTCAAATATATAAGTCT
ATGAGGGACATTTTCTTTCGAACCACTAAACAGAGGGATAGAAAAGCCGGCGCGGACTCCATCTGTCGGTTGCCGTGATAGATAGAACATACTT
CTTTGCCTTCTACTTCCTGTCCCAGCTACATCTCCAGCTGACTCTTGGGTGCCTGCCCACACTGAGGGCGGGGTCTTTTCCCTATTATTTTGCT
GACCTGTAGTCAGTCTTCTCAGGCAATGTGCTTACAGATACACCCAGAAGTATGTTTCAAATTCTCTAGGCATCCATTAGAGCCTTCAGTCGAC
ACCTCAAACTAGCCGTCATGATACTCACCAGTCTAATGTATAAGCACGTGCACTGTTGGCCTCGGTGTTGGCCTCAGCATGCCGTTCCAGATGG
AGCAGGTGGGCAGGATGGCAGCTTGATTACAACCACCTTCACACACTCAGCCATCATGCCATGACAGCCCCCTGCTTTCCTGCCACTCCCACTC
CTTACCATCACTCACTCCCCCCTCCACCCCCCACCTGCTGTGGTAGTCTCCTAGTCACCTGATGTCTTCAAGCACAAGAGCTTGTTGCAATGGA
ATGGTTTTCACTTACATCAGGGTCCCTCTTCCCTTCAAGAGTCGGGCATAGAGAGCTGGCTGTGGCAGTGTGCACCTGTAATCGAAAGACTTGG
GACTGAAAGCACGATGGTAGGAGTTCATCCTCAGCTACATAACGAACTTGAGCCCAATTGGGCCTACAGGAGAACACTCCCTCTCAAAATGATT
ACATGGGAACCTCTATTTCTGGTGATCATGCTAGGACACCTGACCTGTCTCAGAAGCTTTGGCCCACCCAACCTCTCTCCTGTGTATGCTAGAC
ACAAAGTACCTTCAGAGTCAAATTCTACTTATTTGTACTTATTCCTTCTACAGTTTGCATTTTATACTGTAGAGGAACTTGAGTGAACACTCCT
ATAGCATCTCTGACACAAATATTTATCTCTTCAGTTTTATACAATGGGTTTCATTGTGACTTTTCCATAGATGCATATAATGTATTTTGATCAT
ATTCACACACACACACACATACACACACATACACACATATCATCCTCTGTGCTCCCACTGATCCCGAGGAGGTAGAGTACGTACCAATCCTTTA
AATTGAGAGAAGAAGCATGTGAGCAAGCTACTGCACAATCAAACTCATGTCTCACCAACATTCTCCTTGCCTTTGCAGAACTGTGTCTGTATGA
CCCACCCGACGTCCCCAATGCCACATTCAAAGCCCTCTCCTACAAGAACGGCACCATCCTAAACTGTGAATCCAACAGAGGTTTCCGAAGACTA
AAGGAATTGCTCTATATGCGTTGCTTAGGAAACTCCTGGAGCAGCAACTGCCAGTGCACCAGCAACTGTAAGTGTCACCCTTTTGTAACCTGGA
TGGGAAACGATCTAGAGATAAAAGGTGACAGACTCCAGCTCCCATTAATTTGGGTCAACAGATAAAAAATGTGGATAAATCTATGAATAACAGA
TTTTAAAGGCTTCTGTAATATCCACTCACCTAACGTTATTTAATGACGTCTACTTGGTCTAAGAGCTAGGCTTACTAAGTTCTTGACTAAGCCT
GAGTCAATCTGATAGATGCACGACTTTGCTGTTGGCTTCCCCTCTTCATTGAGTTCAGACTCCTCCTTCACACAGCCTGTATCCAGACTCAGTT
TCTTCTCTATTTTGCGGTGTCCATCTATTTTTTCATTGCATATATCTATTATATATAGCACTGTGTTACATAACAATATTATCATGCAGGTATA
AACTGTAGAATGAGCACTTTCTGCCCTGACCTGAAACAGCCACTCTCTTCTATATTCCCCCTCCCCTCTGTTACTAACTTTTAGATTCCTTAGA
CAGTTTTCTTTATACTTTTGGGTTATATAGACATACTTATGTTTAGTTACATAGACAAAGTCTACCAACTGCAAATGAAAGAAAACATATTTCT
CTTTCTGAAGCTGGCTTAATTTGCTTAATATTGTTATCTCTAGGTGCATTCATTTTCTTCTTTATGGCTGAAGAAAATTCCATCCTAGCTGCTG
AGCTGAGGGTAATGGCACATGTCTTTAAACTCAGCACTCAGCACCCAGAGGCAGGTGGATCTCTGTGAGTTTGAAGCCACTCTGGTCTATATAG
TCAGCTCTAGGCAAATCAGGACCAAATAATGGGATCCTGACTCATGTGTGTGTGTGTGTGTGTGTGTGTGTGTTTATGTATACATATATTAAAG
AAAAATCCATATATTCCACATTTGCTTTAATCATTCCTCTACTGTTAGACACCTAGGTGGGTCCCATAACTGATCTGTTGTTCCAGTCTCCTTT
ATTTTTGACCATGAGGATGGAATGGGTCAGTGAGCCAACCTATGGGTGGAGGGCAGAGTTTAAACATGACTGCTCTATTGAAGGGCTTGGCTTC
CTCATTTTCCAACCATGTCCCTTCATCCAAAGCTGGATTTCTATTCTCCTCTCACTGAGGTGCTTGCCTGTAACCTCTCTGCCAGTTTTTCACA
AGGGCCAGAGTTTTCTGATCATTGCCTTAAGAGCACATTTCTCCAACTTGAACTCCTTTGAACCAGGAGAGGGGAGTGGAGGAGTTAAATAAAC
GTTGTGGAGTTAGATTACTCCTACCTGTCATGTGCCACACTGGTGCCACTACTGACAACGAACAAAGCCACTTATGTTTGATCCACATCCACAC
AAGGATTTTCTAGGAGAGAATCTTGCATTTTTTTCTAGGAATGTGTTGTATCCAATGCCAATATGTATATGAGTATGGAGAGGTCAGTGCTAGT
CTAGGTGTGCTCGCCCTATCCAAGATGCCCTACTCACCCCTGCCTGGCTGGGGTGCCTGGGCACAGATGACCTGCTGCTCACGGGAGGCAGAAG
GAAAGGCAGAATGATGGTACACAGCTCATCTTTTCTCTGAAGCGCAAACGGCAGAGTCTGGGATAGATGCCATGTTTCCCACTCACCTGTGTAT
ACCTGGGTGCCACAGGTGGATCACTGTGAGTCAGGAATGTGGGTCCCCCAGTCCACACTTCCCCCTTAGGGTGCCCATATGCCAGGCAGGAAGG
GGAACGCACACCCCAAGTCAAACCCGGTTTGATTCAGAGTGAGTGCTGGGACAATGGAGTCGCTGGAACAGAGAACGCCTTCTTTAGAGATTCA
GGTGTGGCTCTTTAAGACCACGGAGATGATCTTTGTCTCTTCATACTGGTTTATTTGATGGTGGGTGTCACTACATACACTTTAGTCAGGGTGA
ACCAGGGATTAAATCCCTTTTGCCGGAAGGAAGGAATACCCAGGAAGAGCAGCTCATTGGCTGATGGCTAAGGGATACCTCATTAGCAAGGAGA
AGAATTCCAGGTGCGTATGCTATGTGACTCAGTGGCCATGGTTCTCAGTGGAGTGTCATGGTTACATATTCCAGAGCAGGTAGAGGCAAGCAGG
GAATGGCTTCACTCCTGCCATTCTCAATTCTGAGATTCCCGGGATTTAAGCTCATTCAAGCTTGCCATTTCTCATGCCTGTCTGGTAACAAGGT
CCCTCATGCCCTACACAGGAGCGGTCTTTTGCTTATCTTTGCACACCCAGCACAGATATGTCTCTGGGCACCAGAGAGGTGTCCCAGGGTAAAT
GACCGCATAGACAGCTCTTTCTATTCTGAACATTTGGGAACTTACTTGCCTCATTTCTCCCATATTCTCCAGCCCATGACAAATCGAGAAAGCA
AGTTACAGCTCAACTTGAACACCAGAAAGAGCAACAAACCACAACAGACATGCAGAAGCCAACACAGTCTATGCACCAAGAGAACCTTACAGGT
AAGACGTGCATCTCAGCAACCATCCTCTACCCCCCTCCTGGGAACTGAGAGAGATTAGTCCTGTTCTCAACAGCCACGTGCAACCCATTCTCCT
CCTCCTCTCAGATTTCCATGCACATGACAGCATTGCTGATTGTTGGCAGGCTCGCTGCTTGGGAGCATAAGAATTCAACTCCTATAGACTCAAT
TCAACTCAAAACCATGTGCTGGGAATATCTGCCATCTAAGCACCACCCACAGACGAAACACATCGAATAGACAAGGGCCTCTGCCCTTACAAAT
TGCTCCTATTTTCTCTTAAGCACATGCCAATGGACTTCAAGCAAAACCATTGATAAAAATTTGCCTTTTACAGCACAGAGTTCTAAAATGGTAA
CCCCACACACTTATTCACTACTTAATGTGGTGTGGATGTGTGGGCTCTGTGTCTACATACAGTTTCCCAGATGGAATGATAATACTTAAATTAA
GCCATAATGTCAAGAAGAAAGAGGCTTACTAGTGTTAGAGCCGGGGTTTCAGAGATCAGTCACCCTTAAGAAGACCCTAATAGTCTTATCATAA
AGTTATAACCTTACAAAGAACATTATTACATCATGTATAGTCCATCTGAACCCACAGCTGAGTGAACCAGCGAATAGGAAGATGTTCCTTGATT
ACCAAAGAAACTAAGACACAGAGCCTTGAAACGGCCCAGCTACAGTCACATCACAGAGGGAAAGCAAAACCTCTAGTCCCCCTTTCTTCTGAGA
CCTTATTTTTAGCTACTTAAGAACTAGTCACAGCATCCAGTATTGGGGTGGGGGGATAAAGTCCAATCTAGAAGCATTATTGCAATGTGATGCT
TTCATTTCCCTTTCTTTCCCAGCTCTGTCCTGGAATAGTGTTTTTCAAATACATTTAATGTTTTCTCTGCTCTCCTGGTATCTGGTGCCAACCT
CGACAGTTACTGAAAGGTCTCTGTTGGTGGGGTGTCAATATTTGTTCTTACTCAAAAGACAATCAGATGAACTTCGCATGGTTGTACATGTCTG
TAATCAAGCATCTGGGAGCAGACGCAGGAAGATGAGAAGTTCAAAGCTGTCCTGAATTGTATGATAACCTCTTCAGCCCCAAATAATGAGGGAC
GGAGAGAGAGGGGAAAAGGAAGGGAGGGNNNNNNNNNNNNNNNNNNNNNNNNNNNNNNNNNNNNNNNNNNNNNNNNNNNNNNNCCCTTTTTGGC
CCTGGTATTCCCCTGTACTGGGGCATATAAAGTTTGCAAGTCCAATCGGCCTCTCTTTCCAGTGATGGCCGACTAGGCCATCTTTTGATATATA
TGCAGCTAGAGTCAAGAGCTCCCGGGTACTGCTTAGCTCATAATGTTGTTCCACCTATAGGGTTGCAGATCCCTTTAGCTCCTTCGCTACTTTC
TCTAGCTCCTCCATTGGGAGCCCTATGATCCATCCATTAGCTGACTGTGAGCATCCACTTCTGTGTTTGCTGGGCCCCGGCATAGTCTCACAAG
AGACAGCTACATCTGCGTCCTTTCAATAAAATCTTGCTAGTGTATGCAATGGTGTCAGCGTTTGGATGCTGATTATGGCGTGGATCCCTGGCTA
TGGCAGTCTCTACATGGTCCATCCTTTCATCTCAGCTCCAAACTCCGTCTCTGTAAGGGATGTTTTATAATTTGTAATAGTTTGGGGTTTACAG
AAAAGAAAAATTAAGTTCAAACAGGTTCCATCAACTATAATGTTTCCTGGTCTTAAAATAATTTGTTCTCAGACTAAAACTCAGTCCCAGTGTT
GTTACACAAAGTGCAATGTAACAGAAAATGGCTCAGGTCAGGGCCACCCAGCTTCCTGTGGGGGCAGAGAGCTTGAATGTGGTTCTTTTGGGGA
TTGTTTTGGGTAGTCAGGTGTTTTGAAAATCTGGAAGGTTCTAAACCTACGAGTAATTTATGATTCAGAGTCCGACAGCATATATAGTTCTGTC
TGATTAAATGGAAGGCCATTGATAGGGAATTCCAAGATTAATTCAGAATAGGCCACGATCTCTCCGGCCAAACTTCAGCCAAGCCTGGGTTCCT
TCCAGTGAGTGAGAACTTGCTGGGTGTCGCGGATCTTTAGAAGGGTTGACATGGTCGATGTGGGTCGTGCTTCAGCTGTGGTTTAATCAAAGAT
CTATTTCTCTGAGATTCTCTCAGTGACTCCCCTCCTCCAGACATTAGCTTTGTCCTTGGTCAGCTCCCCCAAGTCCTAGGCTGAATGCTAACAA
TTTGTTTGTTCCTCCTCACGTGTTCAGCAGCTGTACTGAGAGGTCCGTCCATCTGCTCCACCCAGCTGCTGTGTCTCAGCAGAGTCTCAGGGTC
TTTGCAGCATGGGTCAAATGAAAAGCACATTCCACCTGGGCTCCCGGCAAGCATCCCCTCCGCACCTGCCGCTCCCACTGCCGGTCCACCTTCC
TCTGTCTTTCTCACCTCATCACCTATCCGACCCTTGTGGGAGTGACCACCATGTGACTTTTCCTGGGGAGACCTCAACAAACATCTGGTCACTA
GATAGAGCAATGACTGCAAACTAAAGAAATGATTCCACCCAAGTATCATGTAATGAACCAATCAATTCACTGGGCTTACTCTTGGGAGCAAGGA
TAGTTCAAATTCAGCTGCTTCATCAGAAACCCCACCCCAGCTTGGATAAAAACCCTCGTAAGCTGGTTTGCTGGAGGTCCCAGGGGGACCGGCG
GACAGCCCCAACAGTTTGAGTCTCCTTCTCTCCCGTCATTCTAACTCCTTATGTAACAGTAAGAGAAGAGGGGCGGTGAGCTGCAGGAGCATCC
TGTCATCTGTGGGGTTTGTTTATTTTATTTTTCTGAGTCTTAAAGAGCCGCTCTCCAATTCAGGAAATAGCTGCGCATCCCTGGGTTCCATGCC
TTCCTACAATGCCTACATTTCTGACAGCTGGAATCTATCAACACTTGTAGGTTCAGTGAAAGCAAAACAAGTAAATAAGAACCGATGTCCAGTT
TTAGCCACCAGAGTTGTTCTGGGTGCAAGAAGTAACCTAGAGGCATTTGCTGTGTCTTCCCAAACTTGCCTTCAAAATTCTCTTCCTCGTCCAG
GTCACTGCAGGGAGCCACCTCCTTGGAAACATGAAGATTCCAAGAGAATCTATCATTTCGTGGAAGGACAGAGTGTTCACTACGAGTGTATTCC
GGGATACAAGGCTCTACAGAGAGGTCCTGCTATTAGCATCTGCAAGATGAAGTGTGGGAAAACGGGGTGGACTCAGCCCCAGCTCACATGTGTA
GATGAAAGAGAACACCACCGATTTCTGGGTAAGTTGTTCCTTTCTTGTGTCCTTCCCATTCTTTCCTCATCTGGACCACACTGCCATGGGATCT
GGGAAAGCCACGTGGCCTTCTGGGTCTCACCTTTTAAAACACAGAAAGAACACTCGAAATGCTGAGGTTCCCTTAACTCTACATAGAGATGGTT
AAAGGCAGCCAAATGAGGATCTCTCCGGTGCTCCCAACCATACACGATGCTGTCTCTAGGACAGAGTGCTATCCTGTGTCTATTGAACCCAGAG
CTTCCACAACTTCTGCCCATCACCAAGATATCCTATTGTTCACAAGCGAAAGTTCAGAATCACACCCATGACTCTTCTTTGTACTTATATGTCA
TTTGTACTCTGTTCCATGCTCATTACAGTCCCCTTAAAGGTCCCCCTGATTTTCGATAAAAAGTACAAGTGTGTGGGAAGTGACTTGCCTTAGG
ATGGGCCTTCACATGCCCAGTCCTGCTCACATGCAACCTGCCTGTGCAGAGGTCTTCACCCATCATTGTTTGAAACCTTACTACTTCCATCATC
AAAAGGTGCTCATGAGTTTTTCTGGTTTCTTTGGCTACTCCCGTCTGTCACATTTCATGACTTCCTCTGTGGTTCCCTTGGCATCCTCTTTGCT
TCTCAAAACTTCCCTCCAGAGAGTCACCCCCATGGCCAGGGCATACCAATACTTCCTAGCAAGACCCTGGCTTTCTGAGTTAGGGTTACTATTG
CTGTGATAAAACACCATGATCAAAAGCAACTTGGGGAGCAGCACCCTTATTTGACTTTCATCTCCACATCATCATTTATGATAGAAGGAAGTCA
GGAAACGGCAGGAACCTCGAGGCAGGAATTGATATCATGGCCATGGAGTCATGCTGCTTATTGGCTTGCTCCTCATGGTTTGCTCAGCCTTGCT
TTCTTATAGAACCCACGACCACCAGTCCACAGGTGTCCCCACCAACAATGGGCTGACCCTTCCCACATCCATCACTTATTAAGAAAATCCCCTG
CAGGCTTGCCTACAGCTCAATGTTATGGTAGCATTCTCAATTGAAGTTCTTTCTTCTTCAACGACTGATCACAAAGGAACTGTCAAAGCTCTAT
TCGTCTCCATTTGGTAGTGGTGGTGTTCTCTCTCTCTCTCTCTCTCTCTCTCTCTCTCTCTCTCTCTGTGTGTGTGTGTGTCTGTGTGTGTGTT
ATTCTGACCCATTTCCAAGTCAGGGGTGGAATCCAGGGCTGCGTACAAAGTGTGGAGAGAAAGATTTGTCCACAGTTCCATAGATAGCCACTGT
CAGGGTTAGAATTCCCGTTTACATACTTTTCTATGGTCCCTTAACTGTTTTCAGTGCCCTAGTGTGGACCTGTATCCCAGAGACATGCCCCAGA
AAAGCTGCCAATGTTTTACCGGGTATTTCTAGGAACAGGAGCCCCAGATGGTGAGTGGAAGAAGGCATTAGATCCCATATCACCATCTTTGACT
CCAGATACATTGCTTTCTTTCTTCTGCAGCTAGTGAGGAATCTCAAGGAAGCAGAAATTCTTCTCCCGAGAGTGAGACTTCCTGCCCCATAACC
ACCACACGTATGGAGAAAACGTCCACGAACAGACAGACATTTGGCCATCAAGCAAGGCTATGCAAGCCCCTCAAGCGATGAATACAAAGAGGAA
AGGAAGCTAGCCCTACTTGCTTCCTTCTTCTCCAAAGCAAGAATATGGCCATAGCAGCATAATCAACCCAAGGCAGTCTGGGAAGATGGCTCAG
TTGGTAAAGTGCTTGTCATGCAAAGCACGAGGACCTGAGTTTGATCCCCAGAAATCACACTCTATTTTTAAAACCGCTAGTAACGCAGTGATTA
CCTATAATCCTGCCTCTGACCACGCAGAGACATGGTGATCTCTGCAGCTTGCTGGCCATCAGCTACTGTAGCAGAACTAATGAGCTCAGGTTCT
GTAAGAGATCTCCAAGAAATAACATAGTCAATTAAGAAAGACATCTGATGTTGACCTCCCGCCTCCATAGGCACACATGCACACGCCTGCACAC
ACACATGAATGTGTACACCCTCTCTCCACACACACACACAATGTAAGGAAGGAGAAACCACAGGACAGAGGTTGAAGTCTCCAGACACTCACCT
GGCCCTCACATCGCTTCCCTCAACCTGAGTTGTAGCCGCCGGCGCCCACGAATTACTGGGTTCCTGAAAGGAAAGCTCGGCATGGATGGGAAGG
ACGGTGAGACAAATAATGACCCAAGACAAAGTTCTCTGGTCAAGACTCAATCTTTAATTGTAGGGTCTTGGATTTAAAGCAAGGAGGGAGACTC
ATCCCCAACTTTGCCAGGATCTCCATTGCTTTGTCAGGTTCTCCTCAGGAAAGCAAGCTCAGCTGCTCAGCAGGTAGCTTCATGCAGGAAGCTG
CAGATGTGGCACACAATAGACTTTACCTAGGTAGGAAGACTCCACCCTAGGTGGGCTAGCCAGCTGTCGCCGAAATACGTCTGATTCAGCCGCT
CAAGGCTGGGGGAAGGGCGCACTGAGTGGCTACCTACTGGGAAGCCCCTCCTCTCCCAGCCTTGTGCAGTCCTATGCTAGCCATAAAATAACTA
TCCCCTCATCTCTCTTCTCCACAGACTTCCCACAACCCACACAAACAACTGCAATGACGGAGACATTTGTGCTCACAATCGAGTATAAGGTAGC
AGGTGGGCCAGCTCATTTACTTTACGGACAATGGTCAACCATGCAAAGACGTGGGTAGTTTAGCTGGTTGAAACGGCCACTAATTCTTTCACTA
ATGGTTGTAAGGGTAAAGAAGACTTTAATCGCTATCATTGCTCAAAATCCCATGGGTCAGCTGTAGTACGTTGGGAGTTGTATCAGCTACCTTT
TGTGTTGCTGTGACCAGAAGCCTGACAAAACAAACCGAAGGAAAGAAAGGTTTGTTCTGACTCCCAGTTCTGGGATCCAGTCCATCACTATGGG
CAACCCGTGGCAGCAGGAGACTGAGCAGCTGGTCACAGTGCATCTGTGGTCAAGAAACAGAGAAAAGCTAGCCACTCTGGTGTAAACCTTTAAT
CCCAGCACTTGGAAGCCAGAGGCCAGCCTGGTCTGCATAGTGAATTCCAGTATAGCCAGAACGACATAGTGAGACCTTGTTTCAAAGAAATAAA
AGAATTTTTAAAAAGAGAAAGATAAATGTTTGTTCCCAGCTTACTCTCCTTTTTATGGAACCTATGGAATGGGGCCCCACATTTAGAGCAGTCT
CCCTACCCATTTAGCCTAATCTTGATAACCTCTCAAGGACGTGTCCAGAACGTTATCTCTAATGTGACTCGAAAACCTGTCAATCAACATAAAC
CAAGAGCATCATCAGAGTTAAATCACCCAAAACCAGTTTCTCCCACATACATTTTCAAGAACAATGCCTCCCAAACTTTCCTCAGAGACAAAGC
AAGGATCCAGGGAGAGAAAATATAGTGTCCATACGCACCTCAGTTATTCCTGGTAGGAGGCAAAAGCAGGCCTTCCTGAACCACGGAATCAATG
GAAGCTTCTGGAAGATTCACATGTCAGCATTTGGGAAGTGTGAGCACTTCCAGGCGATATCGGTGTGTTTAGGATTTCTTATCTTGTTTCAATA
GTTCCTTTATGAAGGATCATGCTGCTAATGAGGACTATCCTCTGGGCCTTCCTCAGCTTCAGCTCTTCTGTCAAGTACCAGCCTCTTTCTATGG
CTGTTCCTTCTATGCCCCCTCTGCTCTTTGAAGGCTCCTTGATCCTTCTAAGAAATACCCAGGCAGAAGAGGAAAGAAAGAAGAAACTGCAGGC
CCCAGCTGTTCAGGAGGTCCTTGATCACCTACAGAATTTTCTCACCTGATCTACAGATCCAACCCCCGATCCATGCATTCCCAAAGCATGGTCC
TGACCTGAACTCCATCTCCCTCTCTTGACAGTGGCCAGCTGCCTCTTCCTGCTCATCAGCATCCTCCTCCTGAGCCGGCTCACCTGGCAACACA
GATGGTAAGTCCAACACTGGAAGGACCCAGCACCCCAGTGTCTCGTCCCTGGCCTGGCATCGTGAACATTGCCTAGAGCTTGACCTCAGCTGAC
ACGCTGGGAAGATGGCTCCGACGAAAGCATGAGATGGGTGTCCAATTCCTAAAAGCTTTGTTTAAAAAAAAATCGAATGTGATGATGGCATTTG
CCTATAATTGTATAATTCAAGAGCTGAGCAGCCACGCAGAGGTGGATCCCTCATACTCTCTGGCCAGCCAGCCTAGAGAAACTGGTGAACTCCA
GGTTGATCAGGAGACTCTATTTCAAAAGTAATGTGAATAATATACTAAGGAACAAGATTCCAGGTTGACCTCTAGACTCCACAGTCATTTACTC
CCTGACACACGTACTCTTACTGAATGTGTATCCCCGGTACTGAGTGGGTTACATTAGCAAATGTCACCCCTACTCTCAGAGGTCTGTGCTGTTT
AGAGATTAGGAAAACCTAGGCTTCCCGAATGTGCAGATAGCAAGAGTTTGAATCCTATTTAGAGATCTTCAACCTTGCCATCCATCCACCAGGG
CTCTACCAAGGTCGCTGGAACAGACCATGCCACCCCACCCTCCCTCCAACTCCGCTGATCATCCACTAGAAACCAGCATGGGTAGCAACGGATG
GAGAGAGGAGACGGCGACCATGGCTAGACCACGCTTGATGGTATGGATGAAGGTTGCTAGATACTCACAAGGCTTTAAAAAAAACATTCTTGCA
TTTATCATTAAATACAGTGAAAAGCTCCCGGATAGCAAGAGCAGGGGGACGCTCTCTATTCTGTGTCTGTGTGTCTGGCTGTGTCTGTGTGTCT
GCCTGTCTGGGAGTTCCCTTGTTTTTTAGGCCCTAAGTATCTTGGTCAGTGATATCTGCCATGGCTCATATAATGTCTTTTTATGGATAAACAT
ATTTTCAAGCCATGATTAAGTAAAGAGTTTTGTGTGAGACAGAAACCAGAAATCAGAAATCTAGGTGAACATACAAATTGCACGTGCATGTGTG
CACACACAGACACACACACAGACACACACACAGACACACACACACAGACATACACAGACACACATAGACACAGACATACACAGACACACAAACA
CACAGACACACAGACACACACACACACACTGGGGGGTTGGAGGAGGACTGTGTATCATGGTACATTCCATTGATCTCAGCACACAAGAGTTGGA
GACAGGAAGATCATGAGTTTGAGTTTGAAAGCAGTCGGGCTGTATAGAAATTCGAATGCCAGCCTCATAGTGAGAGCTGTCTCAAAGTAAATGA
GTAAATAAGATGCCAAGGGATGTAGTTCATTTGGTAGAGTGAGAGCTGAGCATGCCCACGAACCCCCGACTTGATCTCAGCACCATAAGAAACC
AGGTATAGCGGCATAAGCCTCTAATACCAGCGCTCACGAGGTAGAAGCAGGGAAATCCAAAGTTCAAGGTCACCCTGGGCTACATGAGATCTTA
TATTAAAAAACTAAACACATTATTTTTTAAAGATTTATTTATTTATTTATTTTATGTGAGTGCACTATAGCTGTCTTCAGACACACCAGAAGCG
GGACTTCGATCCCATTTCAGATGGTTGTGAGCCACCATGTGGTTGCTGGGAATTGAACTCAGCAATTCTGGAAGAGCAGTCAGTGCTCTTAACC
ACTGAGCCATCTCTCCAGCCCCAATACATTATTTAAAGAAGAAGAAGAATTAGAAGAAGAAGGAGAAGGAGGACGAGGAGGAGAAGAAGAAGAA
GAAGAAGAAGAAGAAGAAGAAGAAGAAGAAGAAGAAGGAGGAGGAGGAGGAGGAGGAGGAGGAGGAGCACGAGGAGGNNNNNNNNNNNNNNNNN
NNNNNNNNNNNNNNNNNNNNNNNNNNNNNNNNNNNNNNNNNNNNNNNNNNNNNGAGGAGGAGAAGGAGAAGGAGAAGGAGAAGGAAGGAGAAAA
GGAGAAGAAGAAGAAGAAGAAGAAGAAGAAGGAAGAAGAAGAAGAAGAAGAAGAAGAAGAAGAAGAAGAAGAAGAAGAAGAAGAAGAACAAAGA
GTTAAGAGTATAAAACCATCTTGAGAAAAGCCAGATACATCCTCATTAGAGTCAGCTGTCTGAGATGTTCTGTCCCTGTCTTTGTTTCATTTCC
TGTGGGTGATATAAAATACCCCAACCAAAGCAGCTTAAGAGAGAAGGGGCTTCTCTGACTTACAGGTCCAGGATGCAGTCCACCCTTGTGTGGC
AGCTAAGGTGGCCGAAACCCGAAGCCTTTACTCATATCACACCCTCCATCAAAAGCAGGGATCAATGAATTTGCATGCACACTTATCCCAGCTC
ACTTCCTCTTTCTATACAGTCTAGGACCCAAATCCATTTAGACTGGGTTTTCCCACATCCATTACAGTAATAAAAACAATCCCCTGCAGGCATG
CTCACACATGACCTAATCTAGGTAATCCACCATTGAGATTCCCTTCCGGTAACTTTAGGTTCTGTTAAATTGGCAATTAACACTAAATCATCAC
ACGCCTGACGTCAAGGGTGTGCCCCTCCTGCCCGAGCAGGTTCTGGCCAGCCCTGATACTTTAAATCTTTAATAACAGAGTCATACCTATGTGG
GTGAGAATCCAGTTTCTTCCATTCTTTTGTTGTTCATCTGGCCTCCTTCTCATTTTCCTCAACTGGAGCAAGAAGCAGAAGGCATTGAGGTCCA
CATGAGGGTACCCCGATAGTTGCTGTGCTAAGAGTTACTCATGGCCCTGACACTCTACCCATTTCCTTAAGATGTTCTTTTAGAGTTCTGGGAG
ATAAAACGTCATCTAATATCCCTTGACAGTCTAGGCCCCTCAGTCCTTGTGAGACCTTCTTCCTGCTCACCACGAGCATCTACCTGGCATGGCA
TTTTCGTTTGGTGAAGACACCAGACTCAGAACCAGAGGACAGGGCTCTTCATGAGCAACAATAGCTTAGAGTTTACAATGTGAAGCAAGGAGCT
GTCTAAGACTGTGAGATTCCCCTAGACCTCCCAGTAGCGGGGCCATTAGGGAGACTGAAATAGAATCTCAATATGACCCCTTTGGTCAAAGAAT
TTGTCCAAGAAAACAAAACAAAACACTTCTGAAATCCCAGCCACAAGGAGTCCAGCATAACAAGGTCCTGTTGCAGACACATAGGTGCCTGTGC
TCACGTTTGTACATATATATATATTTACATGTGTGTATATATGTCTGTATATATATGAAAAAAGTTTACTTTCTAGAAGCTAATTTCATCTAAT
GCTGCCCTCTTGTGGCCAATTGCAAATCTTACCTCTGAATTCTGAAAATTTCAGTCTTTGCCACTTTGGAACACTTACCAGGAGCTAGGAGGGC
ACTGGCTGGCTCACATTAGTTACGTTTTTAGGAATTTCATGGGTTTTGTTTTGTTTCGTTTCTTTTCTTTGTTTGTTTGTTTGTTTGTTTGTTT
GTTTGTTTGTTTGTTTGTTTTGAGACAGGGTTTCTTTGTGTAGCCCTGGCTGTCCTGGAACTCACTTTGTAGACCAGGCTGGCCTCGAACTCAG
AAATCCACCTCCCTCTGCCTCCCGAGTGCTGGAATTAAAGGCGTGCACCACCATGCCCGGCCTCATTTTTTGTTTTAAATGTTCGCCCTGGGAA
TTTAATCCAAGGCCTTGAGCATGCTAGACACATGTTCTATTCAGCTATGATCCAGCCCTTTGAACTGTTTTGTTAATACTTTTCTCTCTTTTTC
TGTTTCGTAATGTTGATTTTTGGGTGGGATTGGAGCACGAGCATCCGGAAGTTACAGGACAAGTTTTGAGACTTGCTTTTCTCCTTCATTCACT
AGGATTGGTGCCAAACTCCCTTACTCCCTGAGACATCTTTCTCCCCCAGTCGGATTGTTAGGTTTTGTGTGTTTGATTTGGTTTAGAGCAGTAG
TCCTTAGCCTTCCTAATTCTGTGACCCTTTAATATAGTTCTGTATGTTTAATGGCTAATCTTACTCCAACTCAGCAGTTATTAGTCCACAGGAC
TAAATGAGGAAAAATCCACAACTGCCAAGTGTCAGACATTGCACCGCAGGCCAGAGACATGCACAACCCAGCCCTGCTCGTAGGAACTAGTAGA
CTGGAAGAAAAAAAAAACTTCAAATATTTTTGTCGTCCTCAGCATACACGTCAATAGTAGAATGTTTTCTCAGCCCTGGGTTCAATCCTTAGTA
TAGAAGAAAAAGAAGAAAGGGAGATATGGGTGGAAGGAAATACGGAGGGAGGAAGGGAATGAGGGAGGGAGGGAGGGAGGGACATTTGTTGAAA
GTCTAAGATGGAGATATGTAAGCAGAAGGACATTTAATCTACATGGGAATCAAAAGACAGCTGACTGACCTGGTATACTCTGAAGTGTCAGATA
AAACGAGCCACGAAACATCTCCTGGAAGAGGAAGCTCGGGCACCTATGCAATTGTGAATCTGTGTGGTGATGTCTGAGAGAAGGTTGAGTTGTA
GGACATAAAATGAATGGGCACCATGGCCACAATGATACCACACTGCCCAGACAGTCAGAAATACTGTCATGGAAGATGAGGCCGCATTGAGGAG
CTGGGCCAAGATGCTGATGGTGGTGGCACCTCACATCACCTCACCTAGCAGAGAAACCGTTATCTTGGCCTTCTGGGTGATCTGCTGCGTAGAG
GCTTACCGTGGGCAATGTGTTGACAATATGATTGCATTATGGTGGAATAAATCCACATAGAAGTTGGTGACTGTGCTTTTCTGCGTCTGTGGAT
CTGAGTGAATGGAGGAAACAAACCAAGGGGTATAGAAAAGAATTGAGCAAATTAGTATGACTGGTAAGCTGGATAAAAAGATGGAGTATGGAAG
AAGGATGAGTCAGAGAGGGCCCGTGGCTTTCTCAGACAGGGAGCCTCTGTAGCCACTACATCCTACAGTGAAATGCAAATTGGGGGTCTCAAGC
AATGCATCCCTTAGAGGTTAGCCACAAGCATAGCCCAGTAGGACATCTTGCTCAGACCCACTCAGCTCTATAACCGGCCTAGAGTTAGTTGTGT
TGTGAGATGACCTTCTCTTTGTTTGTTTGTTTGTTTGTTTCTCAAAACACTGGGTTGAATTGCCAGTGCACAGAGCAGAGACTGAGTCCACAGC
CAGTGTTGGGCCCCTGGGTGGTTGCTGGAGCTTGAGGCTGTTTTTCGTCAGGTGGGAGGGTAACAAGATTCTGAAGTAGAACTCCAAGTTAGAC
TCTCAGCTAGGGGGCTCAAGGGGACTGTTCAGATGGAATGGGTTTGGGTGGAGACCATGTTGTTCTCAGTATACAATGGCATGCATGGCTGTGA
CAGGGTTATTCTTCTTCCAGGAGGAAGAGCAGAAGAACCATCTAGCAAGCTAGAAAAGTCAGAGCCCAGGCAACCGGATGCGAATCACAAAGCT
CAAGCCAAATCTGAGACGCCAAGCATTCACCTAACGGCTGTTTCCTTCTGATCCCTCGGTTTCTAGAACATTCTGAAGTCACAGGACATAACAG
CAACTCTATCACTAAACTGGACTTTGCCATTGAAGAATAGGATCTAACCACTTCAGCACAGCAGTTCTAAAGCTTTAATGGGACAGAGGGCCCA
ACAGTGCTCTGTGTGTTTTGTTTTTCTGTATATCTGTTGATCGGAGCTGAGATGGTGTGGTCACTTTTCATGTAACATATAGTATACAAAAAGT
AGCTTTAGGTTGACTTCATTGTTACAACCCAGTTTGGAAAGCCCAAGTAAAACTCAGCACTAATGTAAATAATTCCTCCTCCTCCTCCTCTCTC
TTTTCATCCTCCGCTCCATCTTCCTCTTCTTCCTCCTCCTTTTCCACCTCCTCTGTCCCTACCCACCCCCACCCATCCACTTTCCTTCTTCCTT
TCTGCTCTCACAAGCTCATCCTAGCTACACGTGCATGGCTGCCTCCTTTTTCAACCTCTGTTTGCCTAACTGGCTCTTCTGATTTCATCACTTA
CTGATCAGCCTTTAAAACTCTGAGCTGGCAAAGATGACTCTATCTATGTTCTTGGCTCAGTCCCAGAAGGAAACCCCCTTTTCATGAAGCTTCA
GTTTTGACATCCTGAAGAACAGAAACTGTGGCAGAACAATCTTCAGATAACATCAAAACAAAGTGGAGAAGCCACGGGAACTGTGGAGCTCTGG
TATTCAGAAGCCTGTGTCTAGGGTCTGCGCCAGGAGCAGAAGGCTGAAGGAAGTCCCAGGACGTGCACTTAGATGCTTTCCCAGCAGGCCACTC
TAAGCGCTGGTTTCTTTGGGACAGCTGTCAATTGTACGCTCAATTTAGCCTGCACTAATCTGATGCTTACAGGTGAACACTCAACGCACAGGTA
TGGACTTGGTACATACCGTGAAAACACTGGAAAGAAAAGAATACTTTCAAGTTTACAGAAGGAAGGAAGGAAAAAGGAAGCAGAGGTGGTGATT
ATACAAAAGATTAGCTGTAGACTGGATATCCCAGGCATCCTCGGATAATGCCCCCGCCCCAGCACCCTGATCCAGGTCACCAAAGCCTTGTGAG
ATCAGACTGCAGAGCCAGTCTGTCTCTGAGTCAGTAAATGTAGAATTTGGATTTCTCACAAGTTCCTGGCGGTGTCTTTTTTTTTTTTAATATT
TTTTATTAGGTATTTTCCTCATTTACATTTCCAATGCTATCCCAAAAGTCCCCCATACCCTCCCCCACACTCCCCTACCCACCCACTCCCACTT
TTTGGCCCTGGTGTTCCCCTGTACTGGGGCATATAAAGTTTGCAAGTCCTGGTGGTGTCTTTATGCTGATCTCTAGCCCACACTTTCTGAGGCA
CTGGGCTATCCCAGTGTGCTCTCCTCTTCCACAGATACCAAAAGCACCTGGGTTTGATGCTCAGACTTCTGAGCACGTTCTTGTTCAATCTCTT
GCGTAAGATTTCCTCTCAGATGAGTTGAGTCAGATTCTCATGTTTAACACTGTTTTAGGGGATTCACACAAGCCCAAACTATCAGTTTTCATTT
CTGAAAAGGCTGGAAAATTTTATGAAAAACTTTCAAAGGTCAGACAGAGCCATTTTCACTCTTTTATGTGACCAAGTATGAACCAGATCTTTCC
TATCTATGGTCTCCCCTTTCCAAAATATATCTTTTGTGGGGACACGGCAAGGAGGAAAGTTAAATAGAATCTCAAGCTACTAATTTTAGAAAAG
AAAAAAATATTAAACTCTTACTAAAGAGCTGTGGGTAGTGGTACACACCTGTAAGCCCAGCTCTCAGGAGACTGAGGCAGGAGGATTGCAGTGA
GTCAGAGATCAGCTTCACCTACAAGCAAAACCCTGCCTTACCCCTCAACCTTTCCATAAAAACAGTCTTACTTGTGTAAAATTAATTTTTAATA
CATATTTGTGCACGATGTGTGTGCCTGGTGCCCAGGGAAGCCAAAAAAATATGTTCAATGTCCCTGGAATAGGTGGTTATGAGCTGCCATGGAG
GTGATGGGATTTAAACCCCTGTTCTCTGAAAAAGCAGCTAGTTCTCTTAACCACTGAGCCATCTCTACAGCCCCATTAAATTGAATTTTATTGT
CATTACTCAATATGGGAGATGGGGTAATGATAACAATTTTTTTTTTATAATACTAAGATGTTTAGCTATTTTACTCTCCTTCACAAGTGTAGAG
TAGAATTTTCTAGAAGCTACATGCATGATATTATCGCTCTGGTGTTAACACATAATGGATTTATCTTGTTAATAAAAGAATTAGTAAATAATAT
TTTAAATTTTTTCTTTGTTTTAGTTTTAAGATGATTAATATCTATAGATACTAGTGTACATTAAGAAAGCCTTTGGGATCCTCAATCATTTTGC
ATGGTTTTAGTAATTTTTTAATAACATAAAGAAGGTCTGACAGATTATGCTAAACAGCTATTGTGGTATGGATTAGAAATGGCCCCCACAGGCT
CCTGTGTTCAAACATCAGCTAGCAGTGCTGCTTTGGGTTTTGGAACCTTTAAGAGGTGGGGCCTTGCTTAAGGAGATAGGTCACTGGAGGTGAA
CCAGACTTGCTTCCAGTCTCCCTCTCTGCTGTCCCAGGAAGCCATCATATGAGGAGTTTCACAACATACTTCTGCTGCCACAGAGTCCCTCCAG
TAAGGCACGTACTGGCCACTGTGTAGTGTACCCTCTAAAACTGAGCTAGAGCTCCCCTCTCCTCCCTACACTATCTCTGTTGATGCTTTGTCAC
GGTGATGAAGAACATAACTAAGGCAAACACAAGACATAGTTTCCAGACTCACGTGAGCATCTCAGCCAGACTCAGGCACAGCTGCGATGTGGGA
ATTATCAAGCATGAGATCCAAAGCAATGGAAAATGAATCGTTATGACAGAAGCCTACATCTAGTCTTCCCTTCTTCCCATTAGTAATAATAGCC
CGTGTTTTAGAAGAACACATCTTTTTGGTGTTCTAGGTAGCTTATATTGCAAATGTGGCACAATCTAAGAGAAATCTGGGATGAGGGAACCTCA
GTGAAAGATTCTCCTTGATCAGATTGTCCCACTGATGATTGATATCGGAAGGCCCAACCCACTGTCAAACGCACCACCCATTGGCAGGATGACT
GGGGTTATAGAAGCAAACAGGCTGAGCATGAGCCAGTGAGCAAGCCAGTAACCACTGGCTTCTCTTTGCAAGAAGCATGGCTTTCTTCCGTGGC
TTCTGTTTGATCTCTCTCAATGATAGATTGTGACCTAGAAGTATAAGCTAAAATAAACCATTTCTTACCCATAGTTCCATCGGTTGTGGTGTTT
ATTTCACAGCTACAGAAAAGTGAAACACAACATCTTTCTCCCAAGCTAATGGTGTTTGCCAGCCTCCTTCAGCTACCTGTGGGCATGTGGTTAA
GCTCTGAACACAGAGGTGTGAGTAGAAGCAGTGTGTGACGTCTGGGCTGTGCCTTGAAGAGAACATAAGTGGCATTTTCTCTCTTAGCTCTTCA
AACCAGCAGGCACATGGCCTTGTCTTGCGAGCTAGAAATCCCAATGGCAAGAACCCCATTTTAAGTTTGACAAGGAGACAAAGTTGAAGAAGTC
TAGACATTTGTCTGATGCCAGGGAGGCATTGCAACATGGCTGATTGCATTGGACTGAACTTTGGTAAAAAAACAAACCAGTTGAGGCCATGACT
TTTACCTTGTGAGGACAAAATTAACGATCACTAACACCGGTACTGTTACCAGCTATATACAAACCCTCTTCCCATAACAAATTTTTTCTTACGG
TAATACTGGCATCAAAATATTTTGAGAGAGAGAGAGAGAGAGAGAGAGAGAGAGAGAGAACGAACAATTACTTGTTTTGTGTTGAATGACTCTG
CCTGAGAGTTCCCCTCCAGCAGGCAAGTTCCTGAGATGAAATGCATGAGTTCTTATGCAGACATCACCATCTCTCGGGGCTTCTGGGTAAGTCA
TGAATCGAAAATAGAAAGCTTCTGGGGTCCCTAAAGCCCTTCCTCTTATTTCCATTCCACTCCTCAAGCGTGAAGGATAAAATACATTCCCTGT
TCTGCTGTAATATTAAGCATTTGGATTATTTTAGTCTGGTTCCTTGGCCATATATCAAAACCTTATAGTAAGGGGCCAGTGAGATGGCCAGTGG
ACAAAGGTACCTGCGGCCAAGCCTGATGACTGAATTCAATCTCTAGAACCCAGATGTTTAGAGACCTGGTGCCATTAACTCCACATGTGCACCA
TAGTACAGGCTTTTTTCCCATCACACATGCACACACACACACACACACACACACACACACACACACACGAAGCCCTTATTTGTAGCAGCAAGTT
TAGCTACACACACACACACACACACACACACACACACACACACACACACACATCAGTTAATTTTTGACTAGCTCAAAGGCTGAGCACTCCTAAA
CCTCCCTCTGCTAGCAAATGCTCCCTTCTGCTATTCCTGACACTCCTGAACCTTCCCCTGTCACCCCACGCCCAATGTAGAAGTGGCCAGTGGC
TACTCACCTGAATTCTTACATGGCTGGTTGGCTTTCTCTCCTGCAGTGCTTCCGTTCTATCCTTCTCCCCTGAGTGTTCGCCATTCTCTGTCTT
CATTTCTCCCAGTTCCTAGCCTGCGCAAATCTAAAGGTCCCACCTTAGTCTACCTGTCCAGCCATTAGCTTTTGGCTCTTTATTGATTGAACAA
AAACCAATTGCGGACAAGGACCTTCAGCGTTAGGACATGCACATTCCCAATTTGGGGGGCCAGATTAATTCAAAGCATTGGAACCAATCCCCAA
CACTTATATATTAGAGGCAGAACTTGGGGATACAGTCTCCTTGAAGCTAAATTCTTCTTTGGATAAACAACACTACTCGTGACATAGAAGAGAT
AAAAACCCTACAAACTTGGTGTTTTTGTTATTGAGACACCGTCTCACTGTGCAGCCCAAGCTGCCCTTGAGTTTACAGTCCTATTTCAGCTCTC
TGGTATTTCAGGTATGCATCAACTTACCAAGCTATAAACTTGCCTTTCTTGAGCCTTGAACTTCCTATGTATTGACAAATACCCTTGAACTTCT
GGTCCTCCTGTCTCTACCTCCTCACTGCTGGGATCACAGACACATACCACCATGCTCCATTTACACAATCCCCGAGATAGATCACAGGGCCTAG
TGCAGATTAAGCAAGTACTCTGGCAAACCAGACCAAAAAAAAAAAAACCAAAAACCAAAAACCAAAAACCAAAACCAAACCAAACCAAACCAAA
ACAAAACAAAACCTCAGACAGACACCAGTATATTAGGATACTCTTGAATCCTCCCTTTTTCGTCCCCTCCAAAATTGCAACTAGAGTTGTAAGT
TCTGCCCCCCATGCAAACAAACAGTCCAGATAAACTGAACTCTAGTGGTAAATCTCAATGAGGGGGACCTCTGATCCACATATGTCAAGATGGA
GGTTTCCAAACACTACATGCAGAAACTCCATAGCTGAAGTCACAGGATGGCGGGGTGACACTCTGTATTCCAGCTAACAAGCTAGGGAACTCGA
CTGTGTGCACGTCCCTTGTAACCACCTACACATGAACTGGGAAACCTAAGATCCCTCGATGTTAATCTGCTGGGTTGAAAAACATTCCACCTCC
TGTGTGACCAGAGAAGTCGGGGAGCAGGAAACAACAGAACTCGTTTCCTTCCTACCCACTCACAGCCTCTCTGTGATGCCAAAGGGAAAGATAC
AGAGTACTTAAGGCCAAAGCTTGAGACTTCTTGAAGCTTTGTGAGTTGTGGAGGAGAGACAGTTCTAATCCCAGAGTCCTTGTTAAGCTGAGGT
ACCTGAGAATTTAGAATGGTCTAGCTGTGTTTTGAGACCAACCACTATGAATACAAGCCCGGCAGACTGGACTTGGAAAAACAAGGGAAAATTA
GAGACGTCCTCTGAATTTTCCTTTAATAAATATGGCTCAAACAAGATCCAACTGTTCCTACCTGCCCAACTTGTAAGGCATGGTTAGCTCAGGA
CACAAACAGGCATACCTCTCAGGGGAAAGAGCTCAGGGACCCTGCCACAGGCCTGCAGCAAAGTGAGTCTCCAGATGGGCACTGTCCTGGGAAT
TGAATGGACACCCACAGACATGCAGGCTGCTACCTGCTACACTCCCTCTCCATTATACAACATCTTTGACCCCAAGGTCAAAATTTAAACCAAA
TTCGAATTGACTTAATAATTAATCTAGGAAATTCCTGCAGTGGATAAAGGAAATAAACTTGGGATGAAAAATATTCAAGCCAGGACACTGAGGG
CTTAGAATCAGATATTAACCACTATGACTATAAACTATTGTTCTTACCCTAGGTTTGGAGGCCAATCTGTTCCCTTGTCTTAGGAAAAATACAT
AATATTTTGCTCCACAAAGGACACGTTTGGGAGTCATGGCCATTTCCTTTCCTCTCCAACAATTTCCATCCCTGAAGATGTCTTGTGTGTATGT
GTGTGAATGTCTGTGTATGTGTGTGTGTGTGTGTGTGTGTGTGTGTGTGTGCATCAAACACTCAACACGAAGCAACTTAAGGGAAAAAGGTTTG
TCTGGAATTGCTGTTCTAGGGAATGTGGCTCATCTTCGCAGAGAAAGTATGACAAGAGATGTAGAAGAAGCAGGTCACATCTAAAATCTAGAAA
CAGAGGGATACTGATGGTCAGTTTGCCCATGAAACGGTACCAGACATTTCAGATGGTTCTTCCCTATTCGCAAGCATCTTAATAGACCCACACA
GAGGTGCGTTTCCATGGTGATTCTAAATTCAATCAAGTTGAAAATCGTCATTAACAATTACAAATTCAAAGAAATCCTAAAAGACACCTTATGG
AATACAAGGCGAACCCATTATGCTATCTCAAAGCAGGTACGAATGTCCATCCGTCGTTGGTGGTCCACAAGGGGAAATACTCTTCACCACACTC
GTATTTTTAACTGAAAAATACGATCGATGCATGCGTATCCATTCCTGTGCTGGCTGGTTTTGTGTCAACTTGACACACCTGGAGTTATCACAGA
GAAAGGAGCTTTAGTTGAGGAAATCCCTCCACGAGATCCAACTGTAAGGCATTTTCTCAATTAGTGAGCAAGGGTTTGGAAAGGCCCCTTGTGG
GTGGTGCCATCTCTGGGCTGCCACTCTTGGTTCTACAAGAGACCACCCTGAGCAAGCCAGGAGAAGCAAGCCAGTAAGTAACATCCCTCCATGG
CCTCTGCATCAGCTCCTGCTTCCTGACCTGCTTGAGTTCCAGTCCTGACTTCCTTGGTGATGAACAGCAGTATGGAAATGTAAGCTGAATAAAC
CCTTTCCTCCCTAACTTGCTTCTTGGTCATGATGTTTTGTCCAGGAATAGAAACCCTGACTAAGACAATTCCCCAAGCTGAAACTTAAAAGAGA
CAGCACCTGCTTCACCAACTACTCTTTGCTTTCCTTGCCTGGTAAGTTTAAAGATACGTTCTTTTTTCTATCAGTGTTTCAGGGTGTAAAGACC
AATTAGAATATACTCAGTCAATTTTTCATACAATGTAGCTCAAATACAAAACACATTTGTATGCTTACAAACCATGAATGAGCTGTCATACTTA
CTGAACCTCACCTGGCTGGTTGTGATGCATATCCAGACTTCCGGACCCACATGGAAATTACAGCTGTGCTAGCTGTCTTAGTGTTAGAAGATGC
TACTACGTAGGCTGAGGGAGAATTCTTTATAACAGTCTTACTCAGTTATGAGTCCTGTATGTTATACTACTGACCTGGCAGGCAAGATATGCTC
ACTGGTTCAAGAGCTTCATGATTGATGAGCAACTACTTTCTGATTCAATTTGAGGCCTGTTCCATTCAACTCATGTCTGGTACAGTAACCTTGA
TCAAAAGTCAGAGTCCAGGCAGGCCATAGGTACCACTAGAGAAGNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNAAGAAAATGAGCTGTCTATAAACGCTCAGGAAGCCCCCTTAT
TCTTTACAATGGAGTGTTGGTAGACCCTATGCAGGTTGTGAGCATGTAATCACAGCTTCTGAGTGCAATACACATACACACACACATACACACA
CATACACACACACACACACACACACTCTGTGGAAATAGAAAGAGAACTATCTGTAATGATACAAGAGATCAGTGGTACACGACAAGCGAGTAAT
GAGCATGAATATGATGCAAGTGCATCCTATGTACATGTTTATCTGTGAGAAAAGAGACCATCAGCAGCAGGGAGGGGACAAGAGGGTAATGACC
CAAATGCATTATATATACACGTATTAAAATGTCATAATGAAGTCTATCATTATATACATCATTATGTACACAATGAATATATGTATCAACTTTA
ATTTTTTCAAAACAAGAAATTACAGCTTTCTCATTGAATCTACAGTAAATCTGGCCTCATCCACTAGTTGGCAGGAAAGCAAAAAGTCTTGTCT
AGTTTAAACAATTCTAATAGGCATTGCTGGATTTCTAATGAACAAAAATCCTTCCTCCAGATCAGCAAAATGGCTCAGCAGGGAAAGCACTTGC
CACGAAACTTGACAACTAGAATTTGATCCCTGGGATCCACGTGGCAGAAGGAGAGAAACAATTCCCACACGTTGCCCTTTGACCCGCTTCTTCC
AAGCAATGTGGAGACAGCTGGAGAGAAAACTGGCTCCATCCAACTTCACAGTTCTGTAAAGAGTCAGAGTCTACAGAGGTGACCGTCCACAAAG
GCAGTGCTCCCAGATGCAGACACTGATTCAGCCTCACAGCTCACAGTTCATTGTGAAGGGGGAGTCGGGGGCAAGAAGAGCTCAAGGCAGGAAG
TGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNACACTGTAGCTATCTTCAGACACACCAAAAGAGGGCATCAGATCCCATTATGGATGGTTGTGAGCCACCTCATGGTTGCTGGGAAT
TGCACTCCAGAACTCTGGAAGAGCAGTCAGTGTTCTTAAGGGCTGAGCCACCTCTGCAGCCCTATAGCACCCGGGGCAACCTGTGTAGGGATGA
TATTGCCCACAGAGAGCTCAATTGTCCCTCATCGATCATCAATCAAGAAATGCTTTAGAGACTTGCACCTCCTCAGATGAG
MOUSE SEQUENCE—mRNA
AAAAGCCAAGATGACAGACTGACAGCCTGAGCCCTTGTCTTGCATTCTCCCAGGAAGATGCAGTAAAGGGGTTGACCCAATATACTGCAGAGAA
TTTCATCCAGTTCCCTCCTCCATCCTGATCCCATGTGCCAGGAAGATGGAGCCACCCTTGCTGACGTTGGGGTTTCTCTCATTAACCATAGTAC
CCAGTTGTCGGGCAGAACTGTGTCTGTATGACCCACCCGAGGTCCCCAATGCCACATTCAAAGCCCTCTCCTACAAGAACGGCACCATCCTAAA
CTGTGAATGCAAGAGAGGTTTCCGAAGACTAAAGGAATTGGTCTATATGCCTTCCTTAGGAAACTCCTGGAGCAGCAACTGCCAGTGCACCAGC
AACTCCCATGACAAATCGAGAAAGCAAGTTACACCTCAACTTGAACACCAGAAAGACCAACAAACCACAACAGACATGCAGAAGCCAACACAGT
CTATGCACCAAGAGAACCTTGCAGGTCACTGCAGGGAGCCACCTCCTTGGAAACATGAAGATTCCAAGAGAATCTATCATTTCGTGGTTGGACA
GAGTGTTCACTACGAGTGTATTCCGGGATACAAGGCTCTACAGAGAGGTCCTGCTATTAGCATCTGCAAGATGAAGTGTGGGAAAACGGGGTGG
ACTCAGCCCCAGCTCACATGTGTAGATGAAAGAGAACACCACCGATTTCTGGCTAGTGAGGAATCTCAAGCAAGCAGAAATTCTTCTCCCGAGA
GTGAGACTTCCTGCCCCATAACCACCACAGATCTCCCACAACCCACAGAAACAACTGCAATGACGGTGACATTTGTGCTCACTATGGAGTATAA
GGTAGCAGTGGCCAGCTGCCTCTTCCTGCTCATCAGCATCCTCCTCCTGACCGCGCTCACCTGGCAACACACATCGAGGAAGAGCAGAAGAACC
ATCTAGCAAGCTAGAAAAGTCAGAGCCCAGGCAAGCGGATGAGAGCCCAGGCAAGCGGATGGGAATCACAAAGCTCAAGCCAAATCTGAGACGT
CCAAGCATTCACCTAATCGGCTGTTTCCTTCTGATCCCTGGGTTTCTAGAACATTCTGAAGTCACAGGACATAACAGCAACTCTATCACTAAAC
TGGACTTTGCCATTGAAGAATACCATCTAACCACTTCAGCACAGCAGTTCTAAAGCTTTAATGGGAGAGACCCCCCAACAGTGCTCTGTGTGTT
TTGTTTTTGTGTATATCTGTTGATCGGGAGCTGAGATGGTGTCGTCACTTTTCATGTAACATATAGTATAGAAAAAGTAGCTTTAGGTTGACTT
CATTGTTACAACCCAGTTTGGAAAGCCCAAGTAAAACTCACCACTAATGTAAAT
MOUSE SEQUENCE—CODING
ATGTGCCAGGAAGATGGAGCCACGTTGCTGATCTTCCGCTTTCTCTCATTAACCATACTACCCAGTTGTCGGGCAGAACTGTGTCTGTATGACC
CACCCGAGGTCCCCAATGCCACATTCAAAGCCCTCTCCTACAAGAACGGCACCATCCTAAAGTGTGAATGCAAGAGAGGTTTCCGAAGACTAAA
GGAATTGGTCTATATGCGTTGCTTAGGAAACTCCTGGAGCAGCAACTGCCAGTGCACCAGCAACTCCCATGACAAATCGAGAAAGCAAGTTACA
GCTCAACTTGAACACCAGAAAGAGCAACAAACCACAACAGACATGCAGAAGCCAACACAGTCTATGCACCAAGAGAACCTTACAGGTCACTGCA
GGGAGCCACCTCCTTGGAAACATGAAGATTCCAAGAGAATCTATCATTTCGTGGAAGGACAGAGTGTTCACTACGAGTGTATTCCGGGATACAA
GGCTCTACAGAGAGGTCCTGCTATTAGCATCTGCAACATGAACTGTGGGAAAACGGGGTGGACTCACCCCCAGCTCACATGTGTAGATGAAAGA
GAACACCACCCATTTCTGGCTAGTGAGGAATCTCAAGGAAGCAGAAATTCTTCTCCCGAGAGTGAGACTTCCTGCCCCATAACCACCACAGACT
TCCCACAACCCACAGAAACAACTGCAATGACGGAGACATTTGTGCTCACAATGGAGTATAACGTAGCAGTGGCCAGCTGCCTCTTCCTGCTCAT
CAGCATCCTCCTCCTGAGCGGGCTCACCTGGCAACACAGATGGAGGAAGAGCAGAAGAACCATCTAG
HUMAN SEQUENCE—GENOMIC
CTGTGCCTAGGCTGGAGTGCAGTGGCACAATCGGTAGCTCACTGCAGCCTCAACCTTCCAGGCTCAAGGATCCTGCCACCTCAGCCTGCTGAGT
AGCCGGGACTACTGGCACGCACCAACACACCCAGCTAATTTTTAATTTTTTTCTAGAGAATCTGGTCTCACTATGTTGCCTAGGCTGGTCTCAA
ATTCCTGGTCCCAAGCAATCCTCCTGCCTTAGCTTCCCAAAGTGCTGGGATTACAGGTGTGAGCCCCCACACCCAGCCCACTTCCGCTCTTAGG
GAGAGGCGAGTGCATTTCAGAGAGCACTCGATGCTCCCAAAAGAAGAAGTTGACAGAGCCCACGTCCCCTCAGCCCTTCACACAGCCTCCCCTT
GTCTTCCTCTCCTACTCAGCACTGTGGCTGCACTAGGACCCCTCATGATGACCAAGATCCTCATAAAGAGGACAAGAAGTTCATCCAGTACCAG
TCAGGCCAATATCCCAAAATTAGCATAACTGGCAGAAATGCAGAGGCATCAACAACTCCTCTTGACAACAGGGTTGGTAGAAGAATCAAGGGCC
TAATTTTGATGCCTGGCACAGGTTATGGGAGCAGCAGAGTACATGCTGCCCAGCGGCTTCCAGAAGCTCCTGGTCCACCCCGTCAAGAAGCCGG
AGGTCCTGCTGAGGTGCAACACATCTCAGTGTGCTGAGACTGCCCTCAATGTTGCCCCCAAGAACTGCAAAGCCATCCTGGAAACAGTGGCCCA
GCTGCCCATTGGAGTCACCAATCCCAGTGCCAAGCTGTGCAGCAAAGAAAAGGATTAGACTGCTCATGTGCACGTTTTATTTGTTTTTAAATTA
AAAAACAAAAAAAAGAAAGAAGGAGGAAGTGGAGGAAGAGGAAGGAGCAGCCACGACGAGGAGGAATTTACATCAGCAGGCCGATCACATACTA
GTCCTTGAATTCATGGTTGAATGTACTGACCATTCTCCCTACGGACACTTGAAGGTTTCCATCAAACCCTTCATAACATATTGCATAATTGTGG
ACTCTAGACTTGGGCCACAACTGAGTGATTTTTTTTTTCTCAAACCCAATTCCACAAAGTAAAAGCCTGATGAAATACAGTGAAGGCTGATGCC
TTATTAACTGTGTTTCCCAAATCCAGAGCTCTAAAAAGGTTCTGCATACAGTCATTCATTCAATGCTTAACGACTGAGCATTAATTCCATGCTA
AGTACTGAACTCAGCACTAGGAATAAGAAGGCGACCTAGAGGCATATCCTCTCTCTAAAGATGCATAGAGCCTCATTGGAATGATCAGCCGTGT
CTCCAGAGAGCTACAAGGCAGTTTTCAATTGGTAAATGCCCTGAGAGTGATGGGCTTGTGGCATGTGTAAGGGTTAGACAGACCTGGGACCTAG
ACATGACACCACTCCTGACGAATTATGTGAGTGTGGGTGTTTCACAACCACAATGAGATGCAATGCCTGCACTTGTAACATGGAAATAGTGATG
GCATGCCGGCCCCGCCAGATTGCTGTGAGAAGTCAGCGGCAGAGACATGCAACATTCTCAGCACAGTGCTTGCCATGTAGTAAGGGCCTAGTCA
GTGCTAGTGATTCCTTTCAATATTCCTAAGATGCAGATAAGGGAACAGCCCAGAGGAGGGGGAGCACTTCCAGAGGGAGGGATGCGGTGAGACT
TCCTTCAGCAGGGTAGCACTGGAGCTGGGTGTTAAGGAGTGAGTGAGCTTTGGGCTTATGGATTTAACAGAGGAAACCAAGAAAAGAGGAGGCG
GTGTTGCAGGAACAGTGAGCAGTTGATGATTTTTATTTTGTTCTCTGGTCTGCTTGGGAACATTTTTGTGGCAAAGACAGCATGAAGGATAGCG
AAGAATTAATACTGAAGAGATAGGCCAGGGCAGGTTATGAAGGATTTTGAATACCCGGCTAAGAAATGTGGGCTTAATTTCAAAGACATTATGG
ACACTCCTAAAATGTTACGTTGTAGATAAGGGAAAAGTATTCTTCCAGAAGATTAAATTGGGGCTGGGCACGGTGGCTCACGCCTGTGATCCCA
GCACTTTGGGAGGCGAAGTGGGCAGATCACCTGAGGTCAGGAGTTTGAGATCAGCCTGGCCAACGTGGTGAAGCCCCATCTCTACTGAAAAAAA
AAATTAGCCGGGCATGGTGGTGGGTGCCTGTAATCTCAGCTACTTGGGAGGCTGAAGCAGGAGAATCACATGAACCTGGGAGGCAGAGGTTGCA
GTGAGCCAAGATTGCACCACTGCATTCCAGTGGGCGACAGAGCAAGACTCAGTCAAAAAAAAAAAAAGATTAAACTGGAAGCAATGGGTAAGAA
TGGTGGAGTGGAGAGACACATTTGAGTGAGGGAAGCCAGCTGAAAAGCTGTACAGACGTCTAGACAAAATGTGACACAAGCATGCCTTCACCTC
CCTGCAAAGACCACAGACCACTGAGCTCCAAAGGGGGTTTGGAATCCTTGTCCTGGGCCGCCATTCGTAACTCATCAGTGCTGGCTTGAGAGAT
AAACTCTATTATCCATCCCTGAACTAAAATCATGACAGAAGTGGCCAGGGAGCTTTGCTGCTATCCCCCCAGGAAACACGTCCTCCACTCAAAT
GGAAAGAGGACCCTCTGACAACATCTGTGGGACCCAACAGCACTGGTCACCACAAGCCACAAAATGTTAACAAAGTCAGTTTTCAATTGTTAGG
GACGGAGGACTCAGTTCATGATTCATACAAACCAACTGTTCTCTCCCAGTGTTTTCTGGGTGGCACAACCCACAAGTCAACAGTGGCTTGGGAA
CTAGACATTTGAGTAGAGTTGGGTTATTTGATTCATAGTGGATTTTGGTTTTCCACGGGACCCCTGTGCCCTTGTCTAGTAGAATCTGGTGGAA
ATTACAAACTCCAGAAATTCAACTCAGTGCCGCAATAACAGGATGCACCTGTAGATTTCGTAGAATTAGCAGCAGCATTCTTTCAATACCAGTT
TGAGAGAAATAACCCTGTTTGCATAGTGCCAACTGGGGCAGAATCTGAAGTGTCTTGCCTGCCTCTCGGCCATGGGAGGCACCGATCTCAGCAA
CATCTATCCAACCGCCATCCACTTAACAAGCAAAGATGGAAGGCCCTCGTGCTCCAGGCTATCCTGCAATGGTTCATTTGCTTTTGATGGGATT
ATCACTTTGACAGATGTCCTTGCAATTAACTGGGGGCTTTCTGCTTCCAATCCAAATTCCCACAGGTGGATCACTGGCTCTTGGGGAGCAAAAA
CCCTTCTTTGTTTGTTTTTGCTTCTCCCAGAGCCTGGGCAGAGCTCCCCATTCCTGGATCCTACCTTGATATGCACTGAATTGAATTATACTAA
AGGGTGCACCCAAGAGTGCATGAGCTAAGTAAAATGTAATTTGTGGGACCTCATGTGGACATTTCAGGTCCATGTGTACAAGATAAAGGAGAGA
AAATCCTGGGGGACACCACAGGCCTGGGAAAATGACCTCCTACTAAGCCTAACAAGAACATCCAGTACATAAACAGCAGGCTCTCTGCAACCGC
TTCGGTCCTTCCACTGCCACCCTCCCAGAAAGCAAAGGGTTATCTCCTGTTCGGATTTTGGCTCTGAGACCACAGCTCAGCACATCACTCCTCT
AGAGAAAAGAAAGTCTAAGATGAGAGAGTCAGGCTGCTTTCATGTGGTGCCAGCGTATAGTGCAGGGCAGGCCTGATTGCTGAACGGATCCCTG
CAGACGTGAGCGGTCACTTCCCCTTTTGAGGGCCCATTTCTTTTCCTCTTTGAGGAGACTGTGAGCGTCCTCAGTGCAGGGCTATGTTTTAGCT
TCTTGGCTCTCCTCACAGGTTCTAGCATAATTCTTTGATTAATTGCAGTCAACGTTTATTGATGGATTATGTGACAGAGGTGAAATTCTTTGAT
CTGGGTTTCAGCTTTTCCTTTCCCCCACTCCCTCCTTTTTCTTTTTTTTTTTTTTTTTTTTTAGAGGCGAGGTCTTGCTCTGTGGCCCTGGCTG
GAGTGCAGTGGTGCAATCATAGCTCATTGCACCCTTGACCTCCTGGGGTCAAGCGAACCTCAACGTCTTGGCCTCCTGAGTAGCTGGGACTATA
GGCATGCGCCACCACGCTCAGCTTCACCTTTTCCTTTCCTTGTGCTCCCCTGGCAGATATGCTGGTAAACAATGAACCAGTCAAGAGGACAGCA
ACAGGAGGAAAATGGGAGAAACCCCATTATTCGTGGACTAAACAGGCTTCCCACTAGTCCTGGCTCACAGAGGAAGTCATGCTCCATGTTTCAA
ATGTCCTCAAAGCTCGTTCAACTCCAAAATGCATGAAGACAAAATCGGAATTTTACTCAAGATTTTTGTGGGCTGTTGGGGAGAAGAGTTAACA
TTGCAAAGCATATTGCAAACTCTTATCTTCAAATCATTTTAAAATTTTCTTCCAAGCTCTTTATTATTTCTCCAGAGCTGCCCTGCTCTTCCCA
CTCTGGGTCCTTTCTCTGCCACTCTCCTCAGCATGACTTCTCCCTAATCGGCCAGCATTTCTTTTATTCTATAGAGCAAGTTTTCTTGCCCTGG
GCACCTCTCATTTGCATGTTATACCACCACACCAAGTGAGCCTCAGAGCACCAGTGTTTTAATCCCTGAAAACAGTCCTTACGGGATTCAGCAC
AGAGGTCCTCTTGGCAGCCTGTAGTCTCCAGAGTGACACGTCCTCAACAGTCAAAGTGAGCAACCTATGGTGCCCATCACCAAGTGGGGCTCAC
CATTTCCCTCTGCGTTCACTTCTCTCCTACCTTCTTCCTTATCCCATCCTTCCTCCTTCCTTTTTTTTTCTGCATTCAATTATTCATGGTTATT
CATTTCATACGGGCATCTGTGCCACTTGGTTCTACAGGCATTTTTTTTTTTTGAGACAGACTTTTGCCCTTGTCCCCCAGGCTGGCGTGCAGCG
GTGTGATCTTGGCTCACTGCAATCTCCACCTCCTAGGTTCAAGCAAGTCTCCTGCCTTAGCCTCCGAAGTAGCTGGAATTACAGGCATGTGCCA
CCAAGCCCGGCAAATTTTTGTATTTTTAGTAGAGACAGGGTTTCACCATGTTGGCCAGGCTGGTCTCAAACTCCTGACCTCAGGTGATCTGCCT
GCCTCGGCCTCCCAAAGTGCTGGGATAACAGGCGTGAGCCATCGTGCTCGGCCGGTTCTATGGGCATTGTGGAAGTGACCGTTAACAAGCCCCA
AGGGTAAGTAAAGGAAAATGAAAGGTGATTGATTCCTCACCACCTCTCCCCTTTTTTCCTCTACTCTTTGCCCCTGCTCCCCAATCTAGACTCT
AATAAACAGAAATGATTTTTGTTGCAAGCTGAAAACTCGTTCTTGTGGTTGGGGCTAGGGGGGTGGCAAGTCAAGCTTTAGAGTTGTCTGGATA
ATAAATGCTGTCTTCAAGGACTGTCCAACATTGTAGGAACAAGAGAACAACCCAAGTGAACAACTAACTCCTGTTCACAAATGCTACTGCCTCT
CTGCCATCCCCTAACCTCAGCCTCACACACTTGAATTTCGCCAATCCATTTGATGAGCTCAAATCAGTGAATTTTCCTCCTCGCCAAGCTCACA
AATTCTGTTCCCCGCTTCTTGCCCCACATCCAGGCATTACCAACTGCATTTTCCCTTTGCCTGATATTCTGGCATCAAGCCTCCTTGCCATTCC
AATTTATTTCACCTTCCTCCAGTCCTTATGATTTCCTTCCTGAAGAGTATTTCAGCATCTCTATGCCTCAATCTTCCCTGCACACCACACCAGC
TCGGCCTCATCATCACATCACTTGGCTATCTCCCGCCTGGGTTAGACAGCTTCAGTGCCTGAAGTCCATAGATCTTATTCTGTTTTTCGACGTC
CACCTGACTCTGAATCCAGCTGATATTTCTGCCCTTAGCTTCTACCCCTCTCTACTTCTGGTTAACTATGCACCACACTCTGCTTCCTCAGGAA
CCACCTACCAAGGCCGTATCCATCCTTCAAGGACAATACGTGGGCCTTTCCTGATCACATCAGCTCAACAACTTTTCCCTCCTACATTTCAATT
GCTCTTCTTACCATAATCATTAGTATTCACCCCGCTGTACGTCTAGAAAGAAAGTGCTCTTAAACCTAAGCCAAGGCAGTCTAGCTCACAAATT
TGTTGTCCGCTGTTCTGAGCAGTTTCTTCTAGGAAGTACCAAACATTTCTGATAATAGAATTGAGCAATTTCCTGATCAAGTGAGACTCAGCTT
GCACTGTTGACCCCCTGTCCCTGGATGAACCTAGTTACTTTTAACCAAATCTTCCTTTCTTGAACTTGTTCCTTTCTTGAACTTAATCTACCAA
TGTTATCTAGATAACTTTCCTCAAAAAAAAAAAAAAAAAAAAAAACCCTCTACTAGGAAAGAAATCTTTGAACCGGACTTATTGGAAATTACCT
CCTTGCAGCAGGTTTGAAACAAAACTTTGAATTTGCCTCACAAAGAATTTGTCTGAAACTGCTTTAGTATATGCTAGTTATATTTGTATGCACA
TGTGGCTTCATACATAGTGGTTGGACACCCATATGTGTTATCCACTTTGTTAGGTGACGTAAGTTCACAGATAAACTGGACATAGGGCTATCCT
CTAGAGCCCCACAGTTTGACACCTTAACCTGAGATCCTTGGACCTTTGAAGTTCAGAGTCCCCACAACCGCTGGGTGTGGTGGCTCACACTTGT
AATCCCAGTACTCTGCAGGGCTGAGGTGGGAGGATCGCTTGAGTCCAGGAGTCTGAGACCAGCCTGGGCAACATGACAAAACCCCATCTCTACA
AAAATTACAAAAAAAATTAGCCAGGCCTGGTGGTGCATCCCTGTCGTCCCAGTTACTTGAGAGGCTGAAGTCCCAGGATCACTTGAGTCAAGGA
GGTAGAGGCTGCAGTGAGTTGACATGGCAACACTGCACTCCAGAAACCCCATCTCAAACAAAACAGAGCAAAACAAAACAAACTGCCCATAATG
CTCCTGAAACTTCCTACGGATTTATCCAGAGTATGTGCTGATGTGCATTTTTTCCAAGCAGGGAAACCAAAGGGTCTATCAAATTAAGAAAAGA
GTTTTTACCATTGTGGAAGACAGTATGGCGATTCCTCAAGCATCTAAAATCAGAAATACCATTTGACCCAGCAATCCTATTACTGGGTATATAC
CCAAAGGAATAGAAATCATTCTACTATAAAGTCACATGCACCCGTATGTTTATTGCAGCACTATTTACAATAGCAAAATGGAACCAACTGAAAT
GCCCATCAATCAGACTAGATAAAGAAAATGTGATACATATACACCATGGAATACTATCCAGCCATGAAAAGGAATGACATCATCTCCTTGGCAG
GGACATGGATGAAGCTGGAAGCCATTATCCTCAGCAAACTAACACAGGAACAGAAAACCAAATGCCGCATGTTCTCACTCATAAGTCGGAGTTG
AACAATGACAGCACATAGACATAGGGAGGGGAACATCACACGCCAGGGCCTGCTGCAGGGTGGGGGGTGGGGTGTGAGGGGAGGGAACTTAGAA
CACAGGTGACTACCTGCAGCAGACCACCATGGCGCACTTATACCTATGTAACCAACCTGCACGTTCTACACATCTATCCTGGAACTTAAATTAA
AAACAAAAAAGAGTTTTTAATCCAAAAAAGATTAACAGCAACTAGAAGAAAGAAAGAGATATAAACAGTGAATTGCAATGCAGCACCGCAGGTG
TTATGATGGAGTACAAAGGACTGGAAAAGGCCTCCTGGAGGAAGCGACACTCAAGTGTCTTTCCATTTCACCTCTAAACTCATTAAGGGCAAAA
GCTTTGCTACAGCTTCAGTATGAGATCCTGGGCAATCCGTGACAAAATGGGTCTGCTTTTGCACCCCCAACTTCTTCTCACATCCCTGCATCGT
GCCATGCAGCATCAACTGGAAACCTCAGCATCAGCAAACGACGACAGAGCGTTCATCCGTAAGGTGAACCAGAAAAGCCAGTTCAATGACTTGT
TTAACCATGGTCCATCTCAGAACCAAGAGTTGGGCCTCTTATTTACCAGAAAAATTGTGGGGGCTTTGTGATATGGCTTTAAAAAAATCTTGTA
ATTGCCAGGCGTGGTCGCTCACACCTGTAATCCCAGCACTTTGGGAGGCCGAGGTGGGTGAATCGCCTAAGGTCAGCAGTTCGAGACCAGCCTG
ACCAACATGGTGAAACTCCGTCTCTACTAAAAATACAAAAACTAGCTGGATGTGGTGACGCGTGCCTGTAATCCTAGCTACTCAGGAGGCTGAC
GCAGGAGAATCACTTGAACCTGGGAGGCAGAGGTTGCAGTGAGCCAACATTGTGCCATTGCGCTCCAAAAAAAAAAAAAAAATACATTAACATA
AATTTAAATATTTTATAATGACAATCCACATTAACTACTTAAAGCATAAGCTATTTTCCAGGAGAGGCAGCAAGTGCATTCTACTCCCATGCCC
AAGAAGAAAGGACCGTGACTTTGGTGGGAGTACTAGGAGTTTCTACTGGAGCACTTCCCCGCAGAGTGAGAAACGTTCCTAGAGAGGAAGTTAT
ACCTGCTGTGGAATTTAAGAGAATCTTGTCATATTTTGACAAGTTTTTTGAGATGGAAGTCTCACTCTGTCGCCCAGGCTCGAGTGCAGTGGCG
CAATCTCAGCTCACTGCAGCCTGCACCTCCTCGGTTCCAGCTATTCTCTTGTCTCAGCCTCCTGAGTAACTGGGATTACAGGCGCCCGCCACTA
CGCCTGGCTAATTTTTGTATTTTTACTAGAAATGGGGTTTTACCATGTTGGCCACACTGCTCTCAAACTCCCGACCTCACGTGATCTGCCTGCC
TCAGCCTCCCAAAGTGCTGGAATTACAGGCGTGTGCCACTGCGCCTGGCTAATTTTTTTTTTTTTTTTTTTTTAGTAGAGACGGTGGTTTCACC
ATGTCATCCAGGCTGGTCTCAAACTCCTGACCTCAGGTGATCCACCCACCTTGGTCTACCAAAGTGCTCGGATTACAGGCATGAGCCACCAGGC
CCAGTCAACGTGATGTGTTTTGGAACCCTGAATTCCTTGGCTTGCCCCGAGGGTTTTCTTTTTCTTAATATCTTTGCTTGCTTTCTAGTATTTA
AAAAATTGTGTTTTGCTCTAACTATGCAATGGCTTTAAGTCTTAGACAAATTTCCAGGGAGCAAAACACACTCAACCATTTCATAATAATCAGA
AGAGAGCTCTGATCAATAAATAACCAAGACTGAATTTTACAAAATAATCCAAAGTTTAAAACCAAAGCCCACTTTTTGCATGATCCTTTAAGAG
AAAGAAATCTGGAAGCAAAACACCTTATAAAATGACAATGCACTTTCAGGAGCCCAGGGCACTCTGGTGAAATGATGATGGCTAGTACAGGTTA
TAAGCCTTGGGGAATTATTTATGAATTCTCAGCATCCTTCAGTTCGCCGCATCCTTCTCCATTATTTGAATATTGGAGGCTGCCTGACCAGAAT
CTTGTCAGGACTTTGCTCCTTCATCCCAGGTGGTCCCGGCTGACTCCTGAGGACGTTACAGCCCTGAGGGGAGGACTCACCTTATGAAGTGCTG
GGTGAGACCACTGCCAAGAAGTGCTTGCTCACCCTACCTTCAACGGCAGGGGAATCTCCCTCTCCTTTTATGGGCGTAGCTGAAGAAAGGATTC
ATAAATGAAGTTCAATCCTTCTCATCAACCCCAGCCCACACCTCCAGCAATTGAACTTGAAAAAAAAAACCTGGTTTGAAAAATTACCGCAAAC
TATATTGTCATCAAAAAAAAAAAAAAAAAAAAACACTTCCTATATTTGAGATGAGAGAAGAGAGTCCTAGGCAGTTTCCTGGCTGAACACGCCA
GCCCAATACTTAAAGAGAGCAACTCCTGACTCCGATAGAGACTGGATGGACCCACAAGGGTGACAGCCCAGGCGGACCGATCTTCCCATCCCAC
ATCCTCCGGCGCGATGCCAAAAAGAGGCTGACGGCAACTGGCCCTTCTGCAGAGAAAGACCTCCGCTTCACTGCCCCGGCTGGTCCCAACGGTC
AGGAAGATGGATTCATACCTGCTGATGTGGGGACTGCTCACGTTCATCATGGTGCCTGGCTGCCAGGCAGGTAAGGGCCTGTGGGTGCCCCCGG
AATTCCCGGAAGGCTGATGGGCATCCCTCTTCCCAGCCACACAACCACAGGGAGTCCCCAGGTAGATGGTTCCAAGAAGGGAGTTGAATCTTGG
GTTCCACCTCTTGCCTGTGACCCACGGGGACCCCAGTTTATGCCTCACTGTTCCTTGGTCTGTCAAGAGAGCCTGAAATAGCATTAGGTTCTCC
TGTCCTTCTCAGTCCTTGACAATTAATTCTGGGAAGAATAGTGTGGCATGATATTTGGGATATTTGGATGTTAACAGGGTCCCCATGAGCAGGT
TTCTCAAGAAATTATCTTTTATTCTTCAATGAATTTCCTTTTATTCTCTGCACTCCCACCCCCCAACTAAGAAGCTATCAGGAAGCTCTTTAAG
TTCAGGTTAATTTGCTGGAGACATGAGTACTCATATATCAAACACTGGGAGAATTTAAGATCTCAGAAAAGTTTAGCTTCAAGGATGTATACAC
TGCTTTTACGTCTTAGGAAATAACACCTAGGAATTTCCTTACAAGTGCAACTTTACAGAAATTTTTAAAAATATGCATACTCACTTTTAGAACA
TTACTACTTCTCATAATCCTATGTATATACATGATATGTATATGTATACGTGTTTATATATGCAAATTTTAAATTTCATTCCTTGTTTCAAATG
GATTTTCTCGAACATTTTTTTTTTTTGACACTGTCTCTGTCAGCCAGACTGGACTGCAGTGGCGCGATATTGGCTTACTGCAACCTTCGCCTCC
CAGGCTTAGCGATCCTCCCACCTCAGCCTCCTGAGTAGCTGGGACTACAGGCATACGCCACCACACCTGGTTAATTTATTTTTTTGTAGAGATG
GGGTTTTTGCCATGTTGCCCAAGCTGGTCTTGAACTCCTGGGCTCAAGTGATCCACTCACCCTGGCCTCCCATACTGCTGGTACTACAGGCATG
AACCACCGTGCCTGGCCAATATTTTTTTTTAAAGAAATAAAAAACCACCAACTTCTACCTCAAACCACTCTTCCTAATTGAGTGTGAATTTTCT
AATAATGTCTTCCCTTTGCTTACTCAGGAAGACAGATAAGAAAGTTTATAGTGTTTTTTGAAAGTCTATAATGGCAACAAGTAGAATGCTAACT
GGCTGTAGAAAGAATTTATTAAGAAATGAAACATAATGATGCATTTCAGGAGTCTGCTTGCTTGTTTACCCCAAGTGCCCAGTCATGCTCGGCT
GTGTACTGGCCCAGATGTAGGAGTGTCTTTAAAGAGGGCATTTTATTTACTTATTACATTTTTATTTATTTTTTTTTTACTTTTTGAGACAGAG
TCTCACTCTGTTGCCCAGGCTGGAGTGCAGTGGCGTGATCTCAGTCCACTGCAACCTCCACCTCATGGGCTCAAGTAATTCTCCTGCCTCAGAC
TCCTGAGTACCTACGTTTACAGGTGCGTGCACCACCACGACCAGCTAATTTTTGTATTTTTAGTAGAGATGGGGTTTCACCATATTGGTCAGGC
TGGTCTCAAACTCCTGACCTTGTGGTCCACCAGCCATGGCCTCCCAAAGTGCTGGGATTACAGGTGTGAGCCATCGCGCCTGACCTATGTGTTT
TATTTTAATAGGTTTTGGAGGACAGGTGGTGTTTGGTTACATGAATAAGTTCTTTAGTGGTGATTTGTAAGATTTTGGTGCACCCATCACCTGA
GCATGTATGCTGTACCCAATGCGTTGTAAGATTTTGGTGCACCCATCACCTGAGCATGTATGCTGTACCCAGTGCGTAGTCTCTTTTCCCTCAC
CCACCTCGCACCCTTTCCCCTGAGTCCTCAGAGTCCATCGTGCCGTTCTTATAAAGAGGGCATACTTTTAGGAAGGTTTTTCAATGTGATTTCT
ACATCCACACCGTCTTTGCTAACTCAGTCTAAGCCAAGAACAGGAAAATCTTGACAATTAATCATGCTCTGCCTCTGGAAGACACATTTCAAGT
AAACTCGTGTTATTTCTAGCTATTGGTGACTTATCCAAGGGTCTGAGTGGTCTTGGAGGCAGCAGGGGAACTAGAGTCCCATAAGGACTCCTTC
ACGCCATTCTCAACATCTATCCATACCCCATCTGCCCCTCAACACTATAGGAAATACAAAAATCAGCCAGATATAGTCCTGACACTTAAGCAAC
TTATAACCTGGTGAGGAGGAGAAAAACATAGATATAAATATAGTAATACCAGTCAGAGTGTGGTAAGATCTACGGAGCGTTGGGCTATCAGAGA
AGGCTTCATGAGCATCACATCTAAACCGAGCCCCAAGGGACCAGTAAATTTCCATCAATGAGATTCCAGGGAGAGAGACCCCAATCAGCCAAAA
CACAGGAGTGCCTTAGGGAGCAGCAGGTGGTCCAATGTGCTCCTGCCCTGGACAGGGAGGGTGGGGATATGGGGAATTCAAACTGGAAACTTAG
GATGAGGTAAAATGAATCATCTAAGGCATTAGGCATTTTTAGTCTGTATATTGCTGGGCCCTGGGATGTGGTCAGAAAGAGAATTCTCCTAGTG
ATAACTTCTTAACTTCACCTAGTAGGCAATAAAGAACTACAAGAAAATTTAAAACTACCATTTATAAAGTAACTTCTATGTGCCAGATATGTGT
CCTATTGTAATCTTCATAGCCACTTTGCAGTATGGATATCATGAACCCCTAGATGAAGCAAGAACCCAAAACTCAGACTCGCACAGGCAGTGAG
AGTGCCAGGGTTTGAACCTGGTCTCCCTGGATTGTTCTGTTTCTGTTCATTGCAAACAGGGTCAGCGCTTTATGGAGATGATACTGGCATAGAG
TGGAGACAGATCAGGTGCACAGAGACAGGTCATGAGGCTCTTAAAATAGTCCAATCACAAGGCAAATGAGGACCCAGTGGCCAAAGACAATGAC
AGGGGTCAAGTTTCTGGAATCAACAGGAATTTCTAGGCATCACTCCAGGGTAAGAAACCATCTCATCACACACTTTTTCTCCAAGAGGAACCTA
GTCTTGAAACAAATCAGACTTTTTATTCCTCAGGGTAAGAGTTCTCTAATGAAATCAGACTGTCAACAGATGCAATTTCTAGCTGATTCATAAG
GGCAGATTTTCATTCTGAAAATTTCCAAAGCCACCATTTAACATGGCAACGCTTTATGAGGACATATTAGTCAACTTGAGTGTTTTTAGTCTCT
ATATTACTCGGCTCTGGGATGTGGTCAGAAACAGAATTCTCCTGGTATTACCAATATGCCACGTTATTGAAGGCACCTTTCTCAGAACTGTGGA
AATTCTGAGAGCTTGTATGTCCCTTGATCATCTTTCTTTCATTCTACGAATTCTGTGATCAAATCTTTATCCTCTAGACTCAAAAATAATTCTT
TAAAAAAACAGTGTGGAAACCCATTGAGATAATGTGTTTGTGTAATTATCCATTCTTGTAAAGACAACTGCCCACAATGGAATTTCCCATCTTC
ACCTTTGTCAGTCAAACACAAAATGTTTAACCCTTCACATTTAATCTTTCAACTTCTTGAGATCCCAAAGTATTATTGCTACTCTTACTTCATT
CATCTTCAAGACATGTCTTGAGGCTGTAAGGGCTTTCCTAAACATTGGGAGCTCTTCATAAGACAGTTGCTACTTTCCTTAAAACAGCATGCCT
TAAATAGGCAGTTTGATAAATCACCTGTGCATATACTTCATTTTAGATGTGAGAATTGAAAAAGAAAAAAATTGACTTCACAATTCTGCACCAA
AAGAATGAGTTTCACGAATGCGTCAGTTAGAGGTGCTAATATACAACGGTTGCTGAGCTCTAAAACTCCACTACTCCACCCCACTCCCCTGATA
GAGAAGTATTCCCAGACTGGTTTTTGCCTAAAGCCTTGGGTATTCTGAATACCTCTTTGTGATTTTTTTTTCTCTTCCTTTTTCACACAGGGTC
TCACTCTGTCACTCAGGCTGGAGTGCAGTGGCGAGATATTGGCTCACTGCAACCTCTGCCTCCCGGGTTCAAGTGATTCTCCTGCCTCAGCCTC
CCCAAGTAGCTGGGATTACAGGCACCTCCCACCACGCCTGGCTAATTTTTGTATTTTTAGTAGACACGGGGTTTCACCAAGTTGGCCAGGCTGG
TCTCGAACTCCTGACCTCAGGTGATCTCCCCACCTTCGCCTCCCAAAGCACTGGGATTACAGGCATGAGCCACCGCACCCAGTCCTCTTTCTGA
CTTTTTTGTTGGAGTCATTCTTCTGCGAGCTATGGATATCATCAAGGCAGGCAGTCCCTCAACGTGGCAGCTCTTCCCACCCTTCCCTGCTCCT
CTTCTTGCCCAAGGATGAGCAGAAACTTTACAGAAAAAGAGACAATTCCCTCAGGAAATGAGGCATGTTGCTTGAGGTCACAGGGAATAGAATT
AGCAGGAAAATAAACCTTGCAGGAAGCAGAAAGTGGCCCAAACCACTGAGGAGAGGACACGGGGAGGCCGTCAGTTTTTGACAGATTTCTCTCT
GTAAAATGAGGAGGTCACCCAGGGATCTTGAAACTCCCTTCCAGTTCCTTCACTCTATGTCAAGGATTCCAGAAAGGAAATAAAATAAGCTCTG
CTTAAAAGGCATTTTCAAGCTGGGGAACTTTGGGATGAAAAAGTTTCCCAAATGAAGACATACTGGCACGGTCAGGGTTGAGCGACTTTTCTGT
ATCAATCCACCCAAGCCTGATGTCTCATTGTGGGTTATTTCCAGCTCTGAAGAAGTACTGCCTCGCACCCAGCATGACCCACTCCTTCTGTAAT
TGCTGCAGTTGCAGGTGTCAACGCAAAAACTCCACTCACTCAATTCTCAAAATCTATCCAATATCTCTCATGCCCCTTTTCAATACTATAGGAA
ATGTAAAAATGAGCGACATAGTCCTGATCCTTAAGAGACTCATAAGTTGGTGAGGAGGAGAAAGGCATAGATATAGTCATGGTAACACAAGTCA
TATGTGGTAAGATCTACTGAGCATGGCGCTATCAGAGAAAGCTTCATGAGGGTGACATGGAAAGGGAGCCCTGAGGGACTGGTAAATTTCCATC
AGTGACATTCCAGGGAGAGAGGCCCCAAGACACAGGGGCAGCAGGTGGTCCACTGTGCTCCTGCCCTGGACAGGGAGGGCGGGGTCCTCTGCTG
TTTGTTTGCTTGTGTAGGAATTTTCCCCTCAAACAATGTCCTTGTCTCCCATGGGAGAAGTTTTACATGAGTGGGAACCTGTTTCTCTTCTCTA
TCCCAGGCCATTCCCTGAGAGAAGAGAAAGGGGAATCTTTTTTTTTTTTGGACAGTTTCGCTCTGTCGCCCAGGCTAGAGCACAGTGGTGCAAT
CTCAACTCACTGCAACCTTCAGCTCCTGGGTTCAACTGATTCTCCTGCCTCTGTCTCCTGAGTAGCTGGGATTACAGGTGTGCACCACCATGCC
TGGCTCCTTTTTTGTAATTTTAGTAGAGAGGGCGTTTTTGCTATGTTGCGTAGGCTGGTCTTGAACTCCGAGCCTCAAGCCATCCATCTGCATG
GGCCTTCCAAAGTGCTGGGATTACAGGCATGAGCCACTGTGCCCAGCTGAAAAGAGAATCTTAATTCAGTTGATTACTTCTAAAAGTTATAAGA
CCTTCTTCACTGAAAGAGTTCACATACAACCTCAACTTCAATCCAGCCCTCTTAAATTTCAGTTCAATATTCCCTGCACATCTCCACATCTTTA
CATGAATTCTCAGTGATATTCCTTCATCCATCCATTCATGCATGAGGATTACAGAGATGAAGAAAGTTCCAGCTCTTATCCTCTTGTGCTCACA
GTTTAACACGGGAGATGAAACTGCTGCTCAATGGCTCCCATTCTGAAGTCAGCCAACAGTGTTTGCCAATAGCCCCCACTGAATACCCCACCAT
ATTCTCAAGTAACTCACTCATTTAGGGGAAAACAATAATATAATTGTCTCTAGAAATTTTAGCTATTCTCCACTCTAAGATTACTCAGTGTTTT
GACTGATCTCAAGGCGACTCCGTGGTTTGCTTCTGTCAAAAAGAAAACACAGTTCCCACTGGGAAATGTGTGGAGAGTAATGGTTCAAAACGCC
AGCTGTACATTAGTGTATATCCAGGATGCTTTAAAAAGTGATGGCCAAGACTCATAAGATGAATTAAGCGGACTCTCTGGGGAACAGAACCGAA
CATCACAATACCTTCCCTTCCAGTTCCTTGAATACTTCCAAATCGCACTTAGGATTGAAACTCACCAAATTAGAGAGATGGAATAATGATTTCC
ATGGTTGGTCTGATTCACTAGGAACAGGTATGCTCAAGCAGTACTTCTCAACCTTTAATGCATTTGGAAATCATCCAGGGATCTTGTCACGGAG
CAAATTCTGACTCGGGAGGTCAGGGGTTACCCCCTAAGATTCTGGTGCAAACAGGTTCCACAGGAGGCCTATGTTGTGGGTGCATGAACCCCAT
TTTGAGTAACAAGGCTATCAAGTCAGAGAATAAATACGCATTGCTTTTACCCACACAGAACTTGCGACTGAAATGAGAAAATAGGCTGAGTGTG
ATAGCTCATGCCTGTAATACCTGCACTTTGGGAGGCCAAGGCAGGAGGATCCCATGAGCCCACGACTTCAAAACCAGCCTGGCCAACATAGTGA
GACTCTGCCTCTTAAAAAAAAATTAGAAAAAGGGAAAACCAATCTGGGAAAGATCAAATATTGATCAAACATTGCAAATAATGCCATAAATGAT
CAGAATAGGGAGAGTAACATGCTACCTGGAGAACTAGAAAAAAAATTCTCAGAGAAGGTGGCCCTGGGACCTCCTGGCTCTGAATGGTGGTTCT
GAATATCAGGAGAGGGGCTGGAGAATGGTGTTTTCTAGTGATGCTGCTGGAGTCGTGTACATGGACCGTGTTCCATGACTAGTGAGCTGACCAA
TTAGAGAGATGAGGGGTTTGCCAGGACAACTACATAATGACCCATATTTCAACATACATGCAGTTTTGTGTGTGTGTGCCAACCAAATGAGTCT
CAGATTCTCGGACAAAAACGAATACTTTTGCTCAGACAGACAGCATGTGAGTGCTTCTCCCCCAGCTTCAACCCTGCAGTGTCTACAGGTGTCA
TTGGTCCCCGGTAGACGTCCTCGGTTCAATGGGTCACTGATCACTTATCACCAGCACCACATGACCCTGGATGGCAGCCAAAGAGTTCCTGGGT
CTTGTTTCCCTACCTCCACATTTTTTTCCTGACCCAGCCCGAGCCACGTGCACACCACAGGGCTCGAGTCCCAGTGCTTTTGTCCTGACAGCCC
ATTCTCCCACTCCCACACCCAACCAGTTCCCCGCCGGGTTTTTGACCAGCAGCTCCGCCTGCAGCCCTCCACCCGCATCCAGGCATCCTGCTTA
AGGAGGGGTTCAGGTGGCAGGAAACTCCAGGGCAACAAAGTGTGGCTGGCATGGGTGGGGTTGACCGGAAGGTCACTGTGCTGGCAGCAAGAGG
AACGGGTGGAGGGAAGGCAAGATACGGATCAGGAGTATTGACATCCTGTGCCAGTAGAGGGGCCTGCCCCATGGTGGAAGGTGTGGCCGGATGA
GCTCGCTGGCCCATGTCCCCCCGTTTCCCAGGCTTTTTCCTCCCACGCTGTATGCAGTGCAAGAACAAGCTGGTGCAATTGGACTAGCAGCAAT
TGAGTCCTTTTACCCAAAAGAAGAAGAAAGAGCAATGTTTCAAAACAATAAAAAGGAAGCTGCAGCCAGGTGCAGTGGCTCATTCCTGTAATCC
CAGCACTTTGGGGGGCCAAGAGGGGAGGATCCCTGGAGCCCAGGAGTTTGAGACCAGCCTGGGCAACATAGTGAGACCCCACCTCTATTTTTTA
AAATGATAATTAATTTTAAATAAGGAAGTTTGTGAAAGCCATTGGTTTCTTTATTCTAAAGAGCGTGGAAAATAGGACAGTCTCCAACAGGCCC
TGATTTCCTTTGGTAAGAAAATGAGCAAAGCTATAGACTAGGAAACATGACTGGTTAAAGCCGGCCCATGCACCAACTCCGATACTCATAGTTA
TGTTTCCTTTCGTTCTTATTTGCTTCAAGCTTTGATCCAGAAAACATTTGAACACTACCTAGAACCACCACAGTGAATATCCATTGCCTAAATG
CTATCTTTCAGGTATTATGTTCAGACATAGTATTACTTTGGTTAATTCTCAAAATAAAGTAGGTCATATGATTATCCCCATTTTCTGCCTGGTG
AACTGTGGTTAAATGGGCTTACAGAGATTAAATGGGTTGCCTGATGTAGCCCAACGAGGAAGGGGCTAGGATAGGAAGCAGGCGCTCTCATTCC
AGAACCTGCCTTTTTTCCTCCTGTCTAAACTGCTGCTAAAGGTCAAAGACAAAGGTGCCATGTGAGCAGGCAAGTTATTTTTGTTTTTTACAAA
GGTGAAGTAATAGGCTCTGAGTAATGCCCACTGCTCCTTCAGCCCCAGGTACATGCAGTGAGGCGGTACCCACAGAGCCGCGTGGCACTCCTCA
ACCTGCTAGAGCAGCCGGCCTCCTGCAGGATGGCTTAAGGACCACCTGGTCCTTGGGGTCTGGGTTCTCCGGGTCCTGCTGTTCCCTGCAGCCT
TCCTTCTACCCCCAGAGCAGCTTCGGGGCATCTTTAGAGAAAGCGGCAGTGTCGCACCTGCCCCAGCAGCCACAAAACACAGCAAATTCTCAGC
ACCTCCGGGCGTGAGTCACCATACCACATTCAGGAATTGCCACCTCTGCCCACAGAATCACCCACTCTTTTCTGTTTGGGACCAAAATAGATAC
CTCCTTCAGAAGCCGCTGCTTTCTGTACTCTCAGCAATAAAAAAACAAAGGCTAGGATAGGTGTGTGCCTCTGAGGCGTGAGGGGGGGCGCGTT
TTCTCCCCTGGGAAGGTTTTCAGAGAGAAGTTGTTGCTACAGCAGTACAGGCAGCTGATGGGTTGAGGGTACAGGAGGAAGACGGAAAGTCAGG
GAGATTGGAGACAACCTCTTGTGTGGTAGGACTTCCGGCTATTGGTTTGTTCATTCCTTTGTTCATTCATTCATTCAGCAAATGTTCATTGAGG
AACTGCTATGTGTCAGGCACAGGGTTTGACACACAGAGATGAATAAGAGCCCATCCTTGACCTCTCCACACACAATTATCACACAGGCTAAGTC
CTGAGATACAAGTTTAAAGAAGCAGTCGATGGAACACACAGATCGATGTTCATTACCCCTGCAAAACCCAGCACATCTCACATCAGGGACTGCC
TCATTCACATCATGTTAGATGTGACAGAGAGGACAGCAGAGAGCGTTGTTTTGTCATCAACTACTAAAATTATTAAAACCAAACACCACTAATT
TTGAACTTCAGGTAGACAGAGGATGTAGAGTTTTCCTTTGCTCAAGAAATTCCTTTACAGAGAAAATGGGAACATTTCAAGTGGGAGTATTTCA
ATTCCTTTAATAACAACCTGTTTTCTAGCTCCCACATTCCCCAAGTATTCTAGAAGCACCTTTCATAATGGACAGCTCTGACCAACTGCCCCAC
ACACATCCATGTGAACCGTTCTCCAATCCTAAAAGTCTGTGACTTCTCCAAGGAAAGGAGGAAGCAATCGTATTAGTCCAGTTTACATTATTAT
ATACTTTGTAGCAAAGCAGCAATGTTTCCTTAAAAAATTCTATATATATATATATATATATATATATATATATATATATATATATAGCTGGGCA
TGATGGCTCACGTCTGTAATCCCAGCACTTTGGGAGGCTGAGGCGGGCACATCACCTGAGGTCAGGAGTTCGAGAACAGCTGACCAACATGGTG
AAACCCCGTCTCTACTAAAAATACAAAAGTTAGCCAGGTGTGGTGGCACATGCCTGTAATCCCAGCTACTCAGGGAGGCTGAGGCAGGAGAATC
CCCTGAACCTGGAGGCTGAGGTTGCAGTGAGCCGAGATTGAGCCACTGCACTCCAGCCTGGGTGACAGAGGGAAACTCCGTCTCAAAAAAAAAA
AGAAAAAAAAAAATATATATATATATATTTGTGTGTATTTTCTCCTCATGAAATGAGCTCCTTGAAGCAAATCGGCCCATCTTTTCAGAGAAAG
ATCAGTTTATTACGTCACCAAGTAAAGGGCAAAGGAATCATTTCTAAAAGTTTCGTTTGAAGTCCCCGGAGTTAAAGTTGACGTCAGCCTCTTC
CTGCCCTGGTGCCCCGAGAGTTTCCCGGGAGTTTTGGGGATGACACCCGGCTGCATGCGTGACATCTCGCTGGCTGGGGGCAGCCCTCCTGGCT
TCCCCGACAATGAAACCACGCAGAGCCTGCAACCCAGCTGAGCACCAGTGTGGTCTCTGCTTCCGGTCCTTGTGGTAGCACCTCTGTTTACAGC
CTGATTTAGAACAAAAAGAACTTTTCTAAAGGAGAAAACTCCTTATATTAAATGGAAACCAAAACAAACAATTACCAAAAAAAAAAAAAAGTTG
GAGGGGTGTTTCAGAATAAAAGAGCCGTCTCTCCAGACATCTGTCTGACGCCCAGGTGGCGCTGACACCACAGGGTGAGCCTTTTGGCTGGAGA
TGTTGATCCCTGGTTTACTGCACTTCGGGCTTTCACTACCTTTCACCCACCGGACCATCAGTTTCACTTTCCCTCTCCCAGCGTCTCTCAAGCT
CTGCTTCTCTGGCCCCGTTTTCAATCTACTGCAACACCACCTCTACCCACGCCACCCATCCTCCTCCTCCTCGTCCTGCACTCCCATCCCAGCT
CTGAGCACTCCCGGCCCAGGGAGGCCTTGCTGCGTGCTCTGCTTCTGTGATCTTACAGATTCCTCATGGCCACCATAGGAGATGGGGACTGTTA
GCACTATTCCCCCACCCCAACCACCTTTTTTTTGAGACAGAGTCTCACTCTGTTGCCCAGACTGGAGTGCAGTGGTATGATCTCGGCTCACTGC
AACCTCTGCCTCCCGGGTTCAAGAGATTCTCATGCCTCAGCCTCCCCAGTAGCTGGGATTACAGGCATGCCCCATCGCCATGCCTGACTAATTT
TTGTATTTTTAGTAGAGACAGGTTAGTAGAGACCATGTTGGCCAGGCTGGTCTCAAACTCCTGACCTCAATTGATCCACCAGTCTCAGCCTCCC
AAAGTGCTGGGATTACAGGCTTGAGCCACCACGCCCTGCCACCATTCCCATTTTACGGATGAGGAAACTGAGACCATGAGGCAGTAATTCACTC
TCCCAAGAAAGCAAGGAGCCAGTACATCTGAGCAGAACCCATGCATTCAGGCCGACACAGGAAGCAAGCTGTCAACAGGCACTGTTCATCTCCT
GGGCTTCCTAGGCAGGCCCTGAAGCTCTGGTTTTCAGTCACCCTGTTATTCATCCCCAAGTTAATCTCCCCTGAAAGCACCTGTCGTGATGCCC
TTTCGGCTGCAAGAGCTCCAGTCATTTCCATTGCCTCAGGGGCACTTTCCTACCTCCTCTGCCTGGCACACAACATGCTCCCCACCCAAGACTG
TTCCCTCAGCACATAGCACGCCTTGGCCAGCAGGTCTCCTCCTCTCCTTCAAAGGGAACCTCCTCATTTGTGATTTCAAAGCTGCACTCCCAGG
AACACCCTCCTCCCTTACTGGAACAATCTTCCCCATCTTGTCTGCCTACCGGAATTTTTCCCAACCAGAAAAGCGTCTTGAGAGAATGGAAAAG
TCTTATGAATTTGGAAAGTTGACAGCTTGAATTCAAATCCTAGCTTGAACTTGTGAATTTCTACTTTCCTTTACGAAAACCTTTCCATGCCATT
CTGTGCAGGCACTAGCAAGGTGGTGGGAGGGAGATGGATACAGTAGGGTGAGTGCCGTGTAAAGAAAACGGAGCAAAGTCTGGGACTGCAGAGA
AATGCCACAAACCACAGACAGAAGAGTCCCGGAGGCTTCCCAGAGAAAGATGACACCCAATCCGACACCTCCAGGGAAAGCAGGAGTTAAGAAG
GTAGAAATGCATTAGCGAAGTGTGGCATGAGCATGATGGTCAATTTCAATGTGTCCACTTGCCTCGGCTAACGGATGCCCAGATAGCTGGCTAA
ACAGTCTCTCTGGGGGTGTCTGTGAGGGTATTTCCAGGAGACACTAGCATTTGAATTGAATAGACTGGGTAAATTTCTTGTTGGTAGACTGAGT
AAAGAAAATCACCCTCACCAGTGTGGTGGGGATCATCCAACCTTAGAGGACCAGAATAGAACAAGAAGGCAGAGGAAGGTTGCATTCTCTCTGT
TCAAGCTGGGACATCCGTCGTCTGCTCTTGGACATTTGCACTCCTGGTTCTCCAGCTTTTGGACTCTGGGACTTACAGCAGGGCCCCTCTGCTC
CTCAGGCCTTCAGTCTTAGACTGACCCACACCACCAGCTTTCCTAGTTATCCAGCTCCAGCTGGCATATTGTGGGACTTCTCAGCCTCCATAAT
TGAGTGAGCCAACTCCCATTATAAATATTCTCTTATTTATCTACATACATCCTATTGCTTTTGTTTCTCTAGAGAAATTCTGTTTCCCTGAATA
ATACAATGACCAAGGCCCAAGACAAAACACATGGTGAGCTCCAAGAGCCACGCAGAGTCCCGCAAGGCAGGAGAGCTCAGGCACATAGTGGAAG
CAGAGATTTTGAAGTTGGGCATGAGCTGTGTCTTGGAGTCTCCCCATGTCACTGGGCTTAAACTGTGTCCTGGAACGAACAGAACATCCCAGAG
GGGTCTCAGTCAGGGCATGGCTTGTACTTTGGAGAGACAGCATTGGGTTTATGTGAAGAATGAAGGAAGAAGAGAAAGCACTGGGTTTATGTGA
AGAATGAAGGGAAGCAGAAAGGCAAACCCATAGGGCATTGAGTAACCTCCATGGAAGTCCATGAGGGTAGGGTTTTAACCTCGGTGGGCCTCAG
CTTCTGTTCGTATAAAATGGGAATAACACAGACCCTGCTCCGTCCTGTAAGGCTACATAAGCTAGCCTGCCTAGACTGACCAGAGCACACAGTA
GGTGCTCACTAAATGTGACTTGCTGGGCTTCTAGTCTGCATGGCCAAAGGCCCTTCCCTCGGACCTTTCCGTGTGGATGCCTCACAGGCTGCTC
AGTCTACACATCCAGAACTGTCCTCCCTCCCTGGAACCTACTCTTTCTCCTCTCTTTGCTATCTCAATGAATCCCACCCCATCCTCCCCATCGC
CCAGGAAATCTCGGCAAAATCCTCATTTCTTCCTTGCTGTCCTCCATGCACCTAATAATCCAGCCTCAATTTTTGGCAATTCCAACTTCTAAAA
TCTCTTAACGCCAGCAACCCCTCTTCTTGCTTACTTATCACCACCCACCTCAGGACTCCCATGACAGCCTTCCTGTGATGCATGACGGGCACAT
GACAAGGGCAGTTGAGGGTCACGGGCCACGGCACCACCATCCAGCCAGCACATGAGGGAGGATAGGCGCCAGCACAGGCAGACACTTGTGCGTA
AGAGCACGCACTTGTCAGGCTGCCTGGGTCCAAGTCCTAGCTCCACCACTGAGGGGCTGTGTGTCTCTGGGCAAGTCACTGCGCCTCTCTGTGC
CTTAGTTCCTGCCTCTAGAGAACGGAGATGAGAAGAACACATCACCTCATAATTAATTACTGCATAAGTAATAATTTTAATTATATTGACTATT
ATACTCTATTAGTGTATGATGTCTTTTATTATATATATTTATCATATAATATACTAATTATATTAATGAGTATACTATAGTATTTTATATAACA
TACCCTAATTGCTTATTATATATCTGTATAATTATTTTATCATATAAGATAATTTCATAAGCTAAGATGGTATTTTATATATTTCATTTATTAT
ATATTTATATTTATATACTTTATTCTATTTGAATTGTGATGTCTAATAAAATAATAGATATGTTAATTATCCTTCATATTCATAATAAGCTCCT
TCATTGGTGTCTGGGAAGCCGAGAACATTTGTGCGTGTGAGCTCTCCTGTGTGTTCGGCATGCAGTGTGTTGCGTCTCAGCTGACGTGATTGCT
GTTTTTGGTGGCCCCGAGGCACAGCGTCCATGCTACAGGCAGGAAGAGCTTTGGAGCAGGCTGACTCCTGAGCTCTCCTCTGGTGCCGTGGCTT
CTGAGGCCCCCAGGTCACAACCAGCCTTTGCCATTCTAGTGTTTCCTCTATGTTCATCCCGCGTTCTGAGTGTGGCACAGGCCACACTCCTGAC
CCCACCAGCATCAGGTGCTGTGCACATGGTATGAGGTCGGGATGGAGTGCTGTTGGCATGGGGGTTGTTCCTTGGGCTACCTGTGTCCACATGG
TCTGATTAACCCCTTCTGCACATCACCAAGGGTTCAGCACCTCCTCAGGCTTAGGGCATGCGCAGGGCATGCAGGGATTTGAGCCTCCTTGGAC
AATGCATGTTCTCCATGCAAGCATCCAGAGACATGGAGAGGGTCTTTGTCCCTTTCAGTCCTAACATTCTGTGGCTTCATAACTACTTGTCCCA
CACCCGAGTAAAAAGCAGGATCTTCTCAGCACTGTGCTGACGAAGTCTTTAAACCTTCCAGTAGGACATGGGATCCCTGCCTGCATCTTAAGTT
CCCATGTCCTTAGGAAACGTGGCCACCTTGGAATTCAGCTCTCCAAGAAGCCCAGTGTGAATGCCCATTCCACGACCATCTGAGCTCACCCCCA
CTAACCCTGAGGTATCTGTTGAAAAGAATAGAACCCACCCACTGAAACTATCAGAGATCAAATGTTGTCTGTTGACTAAGAATTTGTGTTAAGG
CAGGGATAGTTGAGCACCAATGCTTTCAAGGAACTCTGGAGAAATGCAAGCCTGTATTTCCTCTCAGCATGGCTGCAGAAGAGGGCCAGGGCAG
CAGACAGGCATTTTGACCCACTCACAGATGAATACAAAGACTAAACTCAGCAAAAGCCTTGAGGTTGCATTTATGTGTCTCAGAACAGTCCCTG
GAGCACAGGCATGGGCTTTTGGTACCACGTAACCTACAACCGTAAGCTCTCTGCTCTATGCTGTCCACTCCATCAATCCCACAGTGCCTGAAAT
GTCATTGTCTTACCCGATGTCTGGGGTAGCATCTGTGAACACACGGATTTGACACTTTTATGATGCTGTTTGTCTCTACTCAACCCTTCTTAAG
CAAAATCACAGTAGACTAGCAGATCATGGTAGTATCTGAAAGCGCCAGGACCCCAGAACCTCAATCAGTGCTCCCCACTTCCCATGGTTTTCTC
CCTGTGTTTCCACATTTGGATTGTCATTGTCTGTGAAAAGGCATGATCCCTCTCAACAACACACAGTGTATGCAATGTGTGTCTTGTGTAAAAC
GGAGTGTGAATTCTTGAGAGCAGGAAGGAAAAGGGGTACAAGAGGTTCCCCTGCGAGATGGGTGAGGACCACGGCAGAGTGAGCCTCGCTGCTG
GGAACCCCTGAGGAGAGCTCTGGAGAGGGTTCTGGACACGTGGGCTTGGTCAGCCTGACAATTAGGATTTGTGTTAACAGGACCGGCCCTCACC
TGACACCAACTGGACTGAGGTTCCCAGGAGAAACACTTAGCATCATGACTTTGGAGGTTGCCATCATTTTTGATGACTTTTTCTTTTTCCTTCT
TTTCTGTTTCCCTCTTGTCAAGTTTCTACCTCTAAGTTATACTTACTTATTTTGTGTTTCTTTACAGTCTCAGGGGAAATTCCGTTGAGTTGAA
AATTTCCTCACCTCCTCCCTCCAGTTTCTCACAAGAGTCTCTCTCCTTGCAGAAAAGACTGTGGGTGGCAGTCACCAATTTAGCATATTTCTAC
TAAAAATATGACTCCATCCCAAGAAATTGGTTTTAAAGATTTGAGGAAAAACTGCTTTTAGATTTCAGAGAAAGAAAAAGAGTGTGTTTCTACT
TCCCCCTTTTAAATCTGATAACTTTTATTTTAATCAGGTTGACATTCTGTGGTGAGCTTGTGGTTAACTCTTTCATGCCCGGAAGCCTGTTGTC
GACCGAGAAGACTCCCAGGATTAGAACATAACCAGCGCCCAACATTTCATTTGCAGAAGCATCTGAGGTCCCAAAATGTGATGGTTTAGCGGCA
GCTGCTTGTAGGGGTATTTTTGGTTTCCTGCTTCTTTTTTCCCTTCTAAGTAAAGACAAAATAACTAGATCCCTCCACCCCACCACCCCATCAT
TTCGTTGTTGTTTAACAAGTGTTGAGTAAGGCACAGTTGTTCTCCAAAAGGGTCTTACCCTGTGAGTTACTCATTTTAGGGTTACGGAAACTCT
GACTATTTGACCCAGTCAACCAGGCCTCAGAAACTGTGGGTCAGATAACAAATAACTCATGGTGCTTCTCTCCTAATTACTGCAGAGATGTGTC
CAAGTGCTCGGAGATCTCCTCCTTGAAGCCAAGGTCTCCTCCTTGAACCACACCGCCTTCTTCTCCTGTCCCGAGCCAATCACCCAACTTTTCT
TTGATGACAGCAACTTCCTTAAATCCCCCAAGCCCTGAGACAGGCACTTTACTTCTCCCACATTTCCTTCAACATTTGTGCTTGCTGAATGAAA
CAAGGAAGGTGAATGTTTGCTTGAATGAACCAATCAATGGGTAACAGCACCAGTGCTAATGCTAATACACTCAACTACTCATTCAGAACCTACC
ATGGCCCAACGCTTGTGCTCTGCTCTTGGGTGCTGATCCAGCAAAGAGTGGGTCGAGGGGGAGTGGGAGCTGAAGTCCCTATACCACCACTTAT
ATTTTGCACTGTCTTATTTTTTCCTCTGGTCACATGCATACATAAATTTTATATCTCTGTAATTATGCCATTTAAAAATCTTCCTACTGAGGTA
GAAGTTGGGACTCAACCCCCGAGGCAGGCCTCAGACATCGGACAAAATTCAGCACTAGCAAAAACAGGATTGCCGCAGAAGCACCTTTCCATAA
GACATGCTCATGAGTGTGCCATGTCAGTTTACCATTGCCATGGCAACACCCACGCCTTCCTGCTCCTTTCCATAGCAGTGACCGGATGACCCAA
AAGTTACTACCCCTTCCCTAGAAATTTCTGCAAACTGCCCCTTAAACTGCATGTAATTAAAAGCACATATAAATATGAATGCAAAACTGCCCTG
ACCTACTACTCTCTGCCTATGTGGTCGCCCTGCTCTACAGAAGCAGTCACGGAGCTGTAGCACAGCCGGAACTATAACATTGCCACTTCAATAA
AGCTGTTTTCTTTTATTCTACCACTGGCTTGCCCCTGAATTCCTTCCTGGGCAAAGCCAAGAACCCTTGCGGGCTAAGCCCCACCTCGGGGCTC
GCTTGTCCTGCATCACTACCACCTTCTCTAGGAAGATGCTCTAGGCTTAATCATTGTGCTGAATTCAGCTGCTGAGGACTGAGAGGTGATTTGG
ATGATTGACTTCATAAATAAAATTGGGAGTTAGACTTTCCCCGCTGTGGACTTGCCACTGACTTGGGGGAGACGGGGGTTTCTTTCTCTGATAA
AGTTCCACATCCACAGACAGAAGGAAAAGGCCCTGAACTAATCACTTCCTTTGGAAGAAGCTTTGGTAAAGAATTAACATTTGGGAGGTTGTCA
AGAAAAACCTATTTGTTCTATAAATATTTAAAAATTATTAAAATTTCAACCAATACAGAAAAACACAAAGATTAATATAACCAACATGTGCCCA
TCATCAGGATTCAGTAAATGTTTACAGTTTACCTTGGTTGTTTCATGTCCTTTTTGTTTATTTTAAATACATGCTCTTGTCGTGCGTGATGGCT
GATGCCTGTAATCCCAGCACCTTGGGAGGCCGAGGCAGGTAGATCACCTGAGGTCAGGAGTTCAAGACTAGCCTGGCCAACATGGTGAAACCCC
ATTTCTACTAAAAATACAAAAATTAGCCTGATGTGATCCACGCGCTTGTAATCCCAGCTTCTTGGCCGGCTGAGGCAGGAGAATCACTTAAACC
TTGGAGGCAGAGGTTGCAGTGAGCCCAGATCTCGCCATTGCACTGCAGCCTGGGTGACACAGCAACACTCCATCTCAAAAAAAAACAAAACAAA
CACAAACAAAAACAAATAAATACATGCTGTAGATACAGTGGAGCTCCCTCTCGGTCCAACCCAATCTCATTCTTCCCCTCCAGTAATTGTTCTC
TTGAATCTCGGCTGTATCTCATGATGCAATATACACTACTCGTTTGTTTTTTATTTTATACATAAATGCATAATATACTGTTTCTATTTGCAAC
TTTGCTTTTTTCACTCAGCAGTGTTTCTGAGATTCATTTTTGATGTTTATTCTTTTTAACAACTGTACATTATTACATTGTATAATAATACAGA
GTTCTTGATAATCCCAAGTTTATTAATCTAACCTTCTTTTGATGGACAATTAGATTGTATCTGATTTTTCCCTATTACAAGCAATGCTCTAATA
AACAGCCTTGAATGTGTGCACATATGCAAGAATTTCTCTAGGAATATACCTAGAAGTGAGATCACTGATCCTAGAACATGGACAGCTTCAATTT
TACTGCTATATTTAAAATGTTCTCTTTGAAATCGCTATGCTAATTTATAATCCTCATCAGCAATTTATGAAATTTCCTGAATTCCTCACATATT
CCTCATCATCACTTACCATAGGGAAATTTTAACATTTTTTCCCAATCTGATAATATAAAATAAAATCTCATTACTATCTTAAGTTTCATTGATA
CCTGGTGATATTGAGAATGCTTCCACATGTTTTTTTGGTCATCTCTGTGGCCTCCTTGTTCATATCCTTTGTCCCTATTTTTATTGGGTTGTTG
GCCTTTAACTTATTGATTTGTAAGTCTTCCTTATGTATTACGGATACTGAGTCTTTGGATAAGTATAGATTCTTGCATCTTCTAAGTAGACGTT
ATTACTTATAAATGACAATTTTATTTTTTGTTATCCACTTCTTATTCCTCTAATTTCTTTTCCTTGTTCCATTGCATTAGCTGGGGTGTTCAAT
ACAGTATACAATAAAAGCAGGAATTGTATATGTTTCCACCATTAAATAGAAGGCTTTTAAGCTATCACGTACATGTTATTTTCTGTAAGCTTTC
AGTGTCTACTGTCTATTGGTTAAATAAGTTTCTTTTTATTTCTACTACCTTAAAAGTTTTTTTTCTTTTAATCAATAATGGATATTCTTTTCAT
CATCATTAGATGCTCTTCTTCATCTATTAAAATGATTGTGTAGGTTTTCTTCCTTTATACCATAAAAGTATTTAAGTACATTGAGAGATTTTCT
AACGTTAAACCATTCACTATTGCTGGGATAAACTCTGACTGATCAGAATTTTTTTTTTTTTTTTTTTTTTTTTTTGCTTAATACATTGCTAGCA
TCAGGTGCAAATGTGTTAACATTCTCCAGTTTGACTGTAGAATTAATTTCTTTTTGTAATTCTTCACCTTATCAACCATATGTATTGTTGAGTG
TATACATGTTCAAGATTATCATAACTTCCAGAATAATTTTTATTTGTGAAAATGCCTTTTTGATCTAGTTTCTCTTATGTTATTGTAGCTATGG
AGCTTTCTTATATCTCGCTACCTTATCAACAATATGTATTGTTGAGTGTTTACAGGGTCAAGATTATTATAACTTCCACAGTGATATTTATCTG
TGAAAATACTTTTTTGGTCTAGTTTCTCCTGTTACTAAAGCTATCGAGCTTTCTTTTGGTTGCTAAATATCTCCATATAATGTGTTTTCCAGCC
CTTTAGTTCAACCTTCACATGTCACTGTATTTTAAAGACGTCTCTTGACATAACAAAAATAGACCTTGTGCAGGACTTTGGGGGAAATTTTTCC
CCCTCAATCAGAATACCTCTGTCTTTTACAAGCAAGCTTAATTGATTTATATTTATTGTACTACATTTGGACTTATTTATGCTATTTTACTTTG
AACTTTTATTAACCATTATTTTTCTTTTACTGTTGTATTTATCTTTTCTTGTCTTCTGTTAAATTGATTGACTTTCCTTTATTTTCTCTTTTCC
TTCTACTGGTTTGTGTATTATATGTTTTAATTCTACTTTTAGAATTAAGTCACCATTGGCCGGGCGCAGTGGCTCCCCCCTGTAATCCCAGCAT
TTTGGGAGGCCGAGGCGGGTGGATCACGAGGTCAGGAGATTGAGACCATCCTGGCTAACACAGTCAAACCCCGTCTCTACTAAAAATACAAAAA
AATTAGCTGGGCGTGATCGTGGGCGCCTGCAGTCCCACCTACTCGGGAGACTGAGGCAGAATGGCCTGAACCCGACAGGCGGAGCTTGCAGTGA
GCCAAGATTGCACCACTGCACTCCAGCCTGGGAGACAAAGCGAGACTCCGTCTCAAAAAAAAAAAAAAAAAAAGAATTAACTCACCCTTTAAGT
TCTCAAAGTACTCGTGATGTTAAAAGTCCACAGTTAATTAACATCTCTCCCAGTTCCAGGATCTTCTGTTCAGAACTTAGCACATTTCTGGCAA
CTAAGGATTGACAACTGTTAAAAAATATCATTTCCCATTATATTTTTGGATTGGTTGTTGCTGATATATAAAAAATGTTGAATTTTATAAATGA
ATTTTCTATCTGGCCACCTTATGAAACTCTTAATAGTTCTAATACTTTAACCGTCTTGGGTTTTCTAAGTACTTAATTAAATTCCTCACATAGA
ATGATTATTTTTATCTTCTCTTTTTCCAACAATTCTATGCCTTATCTATGTTACCAGTTTTATCAGATCACTAATAACGAGGCTTCTTTTAAAG
TCAATTGGAGTGAGAAAAACCCAAAGATGCCATTAAGTCCTTGAGTGTCTTTGGGGAAGAAAAGGTAGGTTTACGAAAAGAGAGACAAAAGGCA
AAGTGAACAACTAACACAATACCATGTATCGCAAATTTAGGTGTGATTTAATGATGGCTAAGAAAAAATTCTAAATTATATATAAAAGTGTATT
CGTATATAAGGTAGTTTAAATAAATAACAGGTAATGTGGGCAGGCAACAACCATCAAAAATGACTGGAAATATTCAAATTCAACCATGGTGATT
TGGTTGGCAGCAATCAGACAATTGACTATGCTTCAGTAGATTGAAATCCGTTATAATCTATTAATGTGAATACAGGTGACAGGAAACAACCGAA
TACAGCATTCTCATAAATAGTTCTTAGAATATGAATTCAAAAGAAACTCTATTTCCTCGTTGTGACACAATCAAGCTTTAAGCAATGAACGCTT
TAATTATTGTTTTTCAGCTTAGAAAGTAACTATATATTTTATCATTGGGTTCTAAAAACAACACCGCTCAAGAGGTCATTGTAGCAACAATCAA
AATTTGTGGAGCATTTTACAGATTTCAATGTCACTTATGTTATTTCAACTGATCCTCATATTTCCATCCTTCATACCCTCACGATTCATGCTTC
AGCAAAACATTCTACCTGTAGTCTCTTGTATCTTCCATATGATGATGCTGTTTTACACTTTTACACATTCTCTGCCAGAATGGCCTGACGTGCC
TAACCTGCCAGTTGAACTCCCACCCACCTTTCAGGCACCTTATAGTTAAGAGCAGCCTTCATTCTGTTGCCTGGCTCATGGGCAGGGGCAGGAT
TCCCAGCAAGGGGTACTATGTCAAGAGGCCTAAAAGGACATATCAGCATGGTAAAAGGAAATGGCTTGGGGTGTATCAGAAGCTCCTATGGTTA
CATGATTGGGAGGAATTCTTTTCATTTCGTGTTACAGACGGAGCATCTGAAATGAGAAGGGACCACAGTGAGCCAGTGAGTGAAAGAGGAGGAA
GATTCTGACACCCAGAGCTTTACCTTCAACATTTCCTTCTGTTTATTCCTTATCTAGACTTCCATTTTCCTACCCCATAGATACCCACTCTTTG
CCCCTAACTTGCCTTCAACTCCTCTACTGCCTTGTAAATGTCCCATGTGTGCTTGAAAAAAGAGTGGGCTCTGCTATTGTTGGAATCAAATTTG
TTAATTGTGGTTATCACATTTGATTGTTCTTACTGATTTTCTGTCTGTCCTATCCATCATAAAGGAAGACACTGTTAAAATTTCTCATTATGAT
AGTAGATTTCTAAATTTATCCTTGTTTTGTCAATATTTGCTTTGTGTATTTTGAAGTTATGTTATTGGGTGCATACAAGTACGTAAGTGTTATG
TTTTCCTGATTCTTGATTTGTATATTTTGAAGTGATGTTTTTGAGTGCATACACATATAGCATTTTTATGTTTTCCTGGTGAATTGAATCTTTA
GCAATACGTACTGACTTCTGTATTTCTATTAATCCTTCTGCCTTGAAGTCTGATGTTAATATAACTCCTTGGATTCAATTGTCCATTCTTTTAG
TTTCAAACTTTTGGAGCATTTATGCTTTAAGCATGTCTCTTATAAACATCATATAACTGCATATTTTTAAAAATATATGCAGTCCAACATTCTG
TAACTGCAGGGTTAATCTATCCACATTGATAATTATTACTGATATAGTTTGATGAATTTCCACTGTCTTATTTTGTTCTTTATTTTTTACCTTC
TCTTTCTATGCATTTCCCCCTTCTTTTGATTTAAGTAAGTTTTTATCCTATTTTATTTTCTTCCAATACCTTGTAAGTTATTCACTTTACTCTG
TCATTTTAGATGTAATTTTTAACATCTTAACATGAATCCAGACTTTACAAACCCTAGATTTAACCAATATCATAGCCTTCCTCTCAAATAATAA
ATAACCTTAGAACAAATTGCCTCCACAAACCTTTTCTACACAGTTCCCTCCTCTTCCTCCTTGCTTCTTTGTGCGCATTTATGTTTGCTATTTG
AAAGAATTTCCTGAAAGTAAAAGTGAGATTTTTTAGAAGGCTGACATGAGGGCAGCAATAGTAGACTTTCACGTGACTCCTGTCCCACCACCTT
GAGCTGTGTGGTTTAGCCAACTTACTTAATCTCTCTGAACCTCAATCTCTTGCTTTCTAAAGTGGGGATGACAGTATCTACCTTCCAGAGCCAT
GGTGAGCATTGAAGGTAATATATGCAAAGAAGCAGCACATAGGCAGCGCTCAGACATCATAGTTATTATTATATAGATAACCCCAAATGGAGGG
CCTGGGCCCTCTGACTTCTTCAAAGATAATAATCTGAACTAAACCACAACATCACACAAACACCTCCCAGCACCCAGATAGTTTAGACATAGCT
CCACCTCTTTGTAAGAGATAACCTTCAACAGATAGGTGAAAAGCACCCTCCTTCCTGAACATTATGATTCTCGGTCTTGGAAAAGCACTGAACT
AGGAGTCAGGGAACCTGGTGATCTGTTGTAATCCAAGGCTTGCCACAAACTTGTGGAATGACCACAGGCCCAGTGTTCTTATCTATAAAATGTA
GAGGTTGGCCTAAATGATCTTTGAGCTCTCTTCTAGTTCTCATATTCTATGATACCATTAAAAGGGATGTTCTACATCCCTTTTGCACCCAAAC
CACTTACTTTCTATACGGCCTAAACTTCTGGTTTGGGTTTCCCAAGGAAGCGAGAGACAACCTTCAATATTACGCATATCTTTGGAGTTCTTAT
TAAACAGTAAACCTCATCTCACTATCAAAGATAGCCTCTGGCTCCAGCGTCCTCTTTCCAGTCTCCCACAGAGATTTCCTCTGGGAGTCAACGA
GCTTAAGGTGAAATTTGGCTAGGGGTCTGGGAGGAATTCTTTTCATTTCATATTACAGACAGAGCATTTGAAATGAGAAGGGGCCACAGGGGGG
CAGTGAGCGGAACAAGAGGAAGATTCGGGTCTCTGACACCCAGAATCTATCTGATTCTGACACAGAAGATGAACCGCGTTAGAGCAAGAACCAG
ATCCCTCTTTACCCAGTGGCCCAAGCTCTTGATGCGTACCTGTGTCCCTTGTAAAGCTCAGTGAAATCATCACCTAGACATTTCCTGTGCTCCG
TGCGTTATTCTAATCTTCACTCCGTACAAGTAGAGCTTAAAGTTCTGATATATGGACAGGAAAGAGAAGAGGGATCAAGCCTTTATCCAGGGCT
GGCTGGGCTAAGAGTAAAACAACTTCACCTGGATTCACCCAAGGGGCAGAGAATCTTATCTCAGACTCGGTCCACATCTTTATCAGCCCCCTTT
TCCCTTTATCTTTCATGTTGCTCACATGGGCTCCAGGGGCCAGATGAGTTTTCTCTTACTTTGAACATCAGAGAAAAAAAATAACGACAAAAAG
TTACTCAACCGACAAACGTTTCTCACCCACACACCTTTCCTCCAGTCAATGACGTTTCCACTGCACCCTGTTAGACGCTGAGTGAGGAAGCATC
TGGGCACAAACCGCTGTGGATGCAGATTGAGCAGCACTCACTGTGCACTGGAGGGATGGGAGTCCTGTCCTCACCAAGGGGGCAGATGGAGAGG
GGGCAGAGAGAGCAGCCGGGGCTTCACTCCCTGGAAACTCTCATACTCCAAGTGCGGCCTGGGGCTCTGCAGCCCCAGCATCATGTAGGAGCTT
GTTAGAAATAGCCTCAATGTCCACACCTGCCCTGACAAATCCCAGTCTGCAAGATCCCGGGGGCTCTGCACACACGCTGCTGCGAACAACCTCA
GCATCCGTCCTGCCGGCTCTAGAGCAGCCAACCGGGATATCCTTCTTCTTCAGCAGTGGTGTCCGCTCTCTAAAATGACTGTCCCCCACATCTC
TTTCCAGGGTTGGTATTTCTGGTTAACTCAAGGGACACAAAAATAGCTCAGGGAAAAATTAATTTTCATTCTACTCCAATTCTAATAATGAAGT
GGTAAAAATTAAAGGGACAGCTTTCAGATTATGGGAGTGCAGCTGCGGGTATGTGGCAAAATCGAAGAAAGACGGCTCATAATTCCACGTCAGG
GAAGACCTGCTCGCCTGCCCAGGCTCCTGTTTATCTTTACTGTCAATGCCGCCTCTTTTTTCTTTGTTTGTTTTGTTTTGTTTTCACTTAACCT
CAGTGGCAAGAAAAATGGGGCCTCTTGCCACGAGGAGCAGAGAGTCACCCGCAAGGCAAAGACCACCTCATTTCACCAAGTTCCTCGTCTTTTA
TTTGAAATCCAAGACATAGAAGCTGCACATTTGTCCTTCTGGCAACAGTGACGACTGACTGAGAGTGGAGTCGAACACTCGAAAGGAGAGAGCA
GGTGCCTGGGAGTCAGAAAAGCTGGCTTGAATTCCTAAATCTGTCGCCGTTTGTCCTGGGTGAGGTCATTCTGGTCATCTGTGGCAGGAGGACT
ACAAATGCCTGGCTTTTCCCTTTCTAGCGGGCACACAAAGAGACAGAGAAGTTGAATCTTTAGCAATCCATTGAAGGCCAAACCCCACAATGCA
AAGAGAACCATTGTCGAGGGCCCTTCACCTGCACCCTCTGTTCTCCCAGACCTGTTCCATGATGCATTCCAGAGGAGGTGGCTCTCCGTCACTG
TCACTTGCGATTGCCAAGGCGTCAGCCAGACAAGGCAAAAGCTCCCAAGGGGTGTTACCCTTCAGCTGCATACAGAACTCCTAGAGGCAACAAA
TCCACAAGGAAAGATACACCCATGATCGTGTATTTCAGCCACAATCATGGACGTCAGCTGGAAAAGGACGAGGAACAAGTGGGATTCTGCCCAT
GTCTGTCTAATGAGCATCCACAGCAAACTCCAACGGAAGATGTGAAACACGCTCAACACTAGAAGAACTCGGTTTGACTCTGTCTTGGTGTGGG
TCCCAGGCCAAAGCCCTCGGCATTTTCAGGATTAATGATCACTGCTGGCTGTCTCTTTTTCCCTCTGCCCACATAAAAGGCACAAAAACTAAAG
AGGAAGGCCACATCACCGCAGAGCCCGTGGTTCAGGATTCTCCTCCTGCGACCCCCGATTTCTTTTCTTTTCTCACCTTAACAGTGTCCAGAGT
ACTTTTATTGCCAGAATAGAGATATAGCCTGCTCAGAGCCTCATGTGCGGCCCACCACTCAGGAACAAAGGGAGAGACAGATACTAAGCATGTG
TGCAACAGAGAAAATCCAGCCCACGAAACCGTGTGCAAGAGCAGCTCCGTCCTGCCTCAATCTGGCTTTGGGAAGCCCACCTAGAACTTGCCCT
GGCGCCAGTCACCCACTCAGAGCCTACTGCCATGAACGCAGGCCCTCACTCTCCATCCCTGCCCACACAGGGAGACCCAGGGCAAACAGAAAGT
CCCCTAGCGGGGGCCCAAGGAACCCCTCGCTCTCTCAGGGTCCCATTCAAGTTTCTGGGCTAGTCTTGCAGACTAAATACCACTCAGTTGCCTG
GTGGGAGTAGGTGCTGGGCAGGGTGGACAGGAGCCCCACTGCTTCTCAGCCCTGTGCCAACCCCTTTGTGTCAGACCCTAGTGCAGGGTGGGGA
GCCCCAATTTCTAAGCCTTGAGTGTTTACCACCCTACACCCCCACCTCCAACTCCCGAATTGATCATCTCAAATTTTCCCACAGTCCATAGGCA
CCAGACACTTCAAATAATCTCAGAAAAGAAGGTGATGTCTACATCCAGAATCGTCCCAATACTTTAAAATTACAGGGAGAAAAATATTCCCCTT
CCCCTCTTCTAAGGAAAATTCTGTCTGCCTCCCAAGCTCCCAGGTCCCTTGGTGTGAGCTGGGGGAGTGCGTAGACCTAGGAATTGGAAAATAT
TCCGGGAGAGAATGATTTATGTTGAGAAGACCCATTGGGAGAAGCAGGAAGTGGGTCTGGAAAGACACGAGCAACTGTAAAGGCAGAGAAAAGC
CCTTGTTAGCTGAATTTTGACAAGTAATTTTTGCCATTGTTAGTGAGTAAAACTAGAGAATGAGGACAGTTGTATGCTGTCTTAAAAAATAAAA
GAATGATAATATAGGGAAAGATGATTTCAACATTTTGGAGTCATATAAGTGGAATCCTCCTGTTATTACTGTGCAAGACTTTTAGACAAATTAT
TTTTTATTCTTTTTTTGGATGTTGTTCTGTTGCTGCTTTTATAACACTACACTGAGAAGGCCTAAAAAAAAAATCCCAAATTTATTCCTATTAG
ATTGAAAAAGAGCAACCCTCCAGCGTACCTTTCCACACACTCTTCTACCTCCTGCTGCCTTCAGGTGATGTTTGGCACTTCCTGACAAGGCCTT
TCAGGGAAAAGGATTATAGCCAAGCTCTTTAGTGGTTACTGACCTACAGACCTGGAGGTGTCAAGATCCTAAATTCCAGGAAAGATTCTTTATG
AAAGGAAAGTCTGTGAGAATAAGAAGTTCTGGAACTTCCCCTTGTCCCCGCATGACCTCTTCAGACACTCGCCCTAATTTATGATGACTCTGAA
AATGCCCATGACCTCATCTAGAAAAACGCACTCTCATGGAATTCAAGAGTTTCTAAGGGCACAAAATAGCTTCCAAGACAATTTGTTTCCTTCT
TTTCAAATTGATGTGTTTAGAAATGTTCTTGGCTTGTGATTGACATCTCAGATTCCAAAAACATCTGAGGTCAATCCTGGTTATTTGTGCTAAT
GAATAATTACAAAAGAGTAGCTAAATATGCTTCTCGCTCATTTTGTTGAGCTAAAATTACTTATAAAGAAATGCATAGATCTGCGGCATACAAT
TTGATGAGTTTTGACAAATGTGTACAGCTGCAAAAACGTTTTCATCACTTCTGCCTTCTGGTCAATACCCACCTACCACAAGAAACCACTGTCT
GCTTTCTATCTCTGGATATAACTTTTGTCTTTTTGTGAACTTCATATAAATGAAAGCAAAGTGTATGCACTATTTCGTGTTTAGCTTATTTTGT
TCAACATAACATTTTTGAGATTTATCAATATTGCCTGTATCAATAGTTCATTATTTTTTATTTTTGAATGGTTTCTATACTATGACTATATCAT
AAGCTACTTGTCTGTTCTCCCAATGATGGATGTTTGAATTATTGAGTTGGTTATTATTAATAAAGCTACTATGTACTAGAATGGAAGGGGCTTA
ATAAAAGAAAGAAGTTACTATAAATATTTTATAAAAGTCTTTCTGTAGACACATCCCTTCATTCCTCTTAGATAAATACCTAGAACTAGAATTG
CTGAATTGTAGGGCAGGTATATATTTAACGTCAATAAAAATACTTTCTTTTTTTGTTTTTTTTTTGTTGTTGTTTGTTTGTTTCTTTGTTTGTT
TTTGAGACAGAGTCTCACTCTGTTGCCCAGGCTGGAGGGCAGTGGCGTGATCTCAGCTCACTGCAACCTCTGCCTCCTTAGTAGAGAAAGGGTT
TCGCCATCTTGACCGGGCTGGTCTCAAACTCCTGACTTAAAATGATCCACCTGTCTCAGCCTCCTAAAGTGCTGAGATTACAGGCGTGAGCCAC
CATCCCTGGCCAAATACTTTCAGATAGTGTTCCAAAGTAATTACATTACTTACACTGCCACCAGTAACATAAGAGAGTTCTGGTAGTTCTACTT
CCTCCCCAGTATTTGACGTATCACTCTTTTATCCATTTCCTCCATTTCCCCAATGGGCTATCTCATCATTGTTTTCAACTTCCATTTTATTGAT
ATCTGATGTTAAATACACTTTCAAGTGTTTATTGATAGTCGTGTATCTTCTTTGGGGAAGTGATTGTTCAAGTCTTTGCCCATTTATTGGGCTG
TTTGCCTTTTACTCTTGCTTTGTTTTTCTTTATATATTCTGCACACAAGTATTTGTCACAAATATATATATTGCAAATATTTTCTCTCAGTCTA
TCATTTACTTTTTCATTTTTTTCTTTTTTTTTTTTTGAGATGGAGTCTCGCTCTGTCACCCAGGCTGGAGTGCAGTGACGCGATCTGGGCTCAC
TGCAAGCTCCACCTCCCAAGTTCACGCCATTCTCCTGCCTCAGCCTCCCCAGCAGGTGGGACTACAGGCGCACACCACCACACCCGGCTAATTT
TTTTGTATTTTTAGTAGAGACAGGGTTTCACCGTGTTAGCCAGGATGGTCTCCATCTCCTCACCTTCTGATCTCCCCGCCCCGGCCTCCCAAAG
TGCTGGGATTACAGGCGTCAGCCACCACACCCTGCCTACTTTTTCATTTTCTTAATGTCTCTGGATGAGTAGACATTCTCAATTTTTTTCTTTT
ATAGTCAGTGCTTTTGCATCCTCTATGAGCAATCTTTGCCTATCTCCAACTTGCAAAGTCATTTGCCTTAATTTTCTTCTGAAATCTTTTTAGT
TTTGCCTTTTATCGTTACATCTATAAGATATAGACCTAGTACATAGATCTTTATCTTTATATATATAATAAAATTAATTCTTGCATAAGATGTG
AGTTTTTTTCTCATACATTTATCCATTTGCTCCAGAAGCATTTGTCGAAAAGATTTTTCCTTTCTCATTGAATTTCCTTGGCACATTTATTTGT
TTAAAAAAAAAGCAGTTGAACAAACATGAGTTCAGTCCATTTCAGGACTTTCTGCTCTGTTTCATTACTGTTTGTCCTTATGCTAATACTACGC
TGCCTTGATTTCCATAGTTTTATAGAAAGCCTGAATATCAGATGTCATGAATCCTCCAAATGTATACATCTTTACGATTGTCTTAGATATTCTA
GGTCTTTTGTGTTTCTAAGGAGCTTTTAGAAATAACTTCCAAATTTGTACTAAAAAATTGTGTTGGATTTTTAAACTCTAGTTCCCTTGAATTA
ATTGATCAATTTAAGGTGAATAGATTTTTTAAATGAATGTTACAATCTATAAAGATAGTATATTTTTCCATTTGCTTAATTCTTTTAAAATATG
TCTCAGCAATATTTCAGAGTTTTCAGTTTAGTGGTATTACAAGTTTTTGGTAGNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNTCACTCACTCTGTAACCCAGGCTGGGGTGCTCGGCTGACTGCAACCTCCACCTCCC
AGGCTCAAGCAATTCTCCTGCCTCAGCCTCCCAAGTACCTGGGACTACAGACATGTGCTGCCACACTCGGCTGATCTTTTGTATTTTTAGTATA
GATGGGATGTCACCATGCTGGCCAGGCTGGTCTGGAACTCCTGACCTCAGATGATCCGCCTGCCTTGTCGCCCCAAAGTGCTGGGATTACAGGC
ATGAGCCACCACACCCAATTTTACGGGTCTGTTTCTATTGAGCACTTTTTTGAGTAGGTCAAGTATGTCACATTTTCCTGTTTCTTCACATGTC
TAGATTTTCTTTTAATTTGCTGGATATTATGAGCTATACATTATAAAGGGTCTAGATTATCTTATGGTCCTTTAAAAGGTAATGCATTTTGCTC
TGCCAGGCAGTTAAATGACTAGCGCATCCTCTTGTCTATTAGGCTTCGTTTTAATCTTTGTAAGGGCAGGTCTATTTTGGTTTTGAAATTCATC
CTAGGGATGCCTCTTTCTCTAAAGCAGGATTGGAAAACTACAACCCACAGCTCAGACACCTGTTTTTATAAATAAAACTTAGCATAACTCTTCT
TCTCTCAGCCCTCACAATGGACAATCAGGGCTAAGTTTCACTGACTTCTGCCCTGTTCAGGTTCAGACCAGCTCTTCGCTAAGAACCTGAGCTG
GACCCCTATACATAATTTTGGGGCGACCTTCATGCAGTGTCCTCTCCAGAGCCCCACCCCACAAATCCCAGTCATGTCACGTCAATGAAACCAC
TCTGCTTGGGTTCCACCTGGGTCCTCCCGGGAAGGAAAGCACCCCAGGAAGGAAGCCAGCCCAACTGTGGGCGCCTCTCACGTCTTTCCCTCCT
CCAAAATCATGTAGCCCTGAGCTGCCTGTTGTTCAGCACCTGAAAGCACTTGCTGTATACATTTGGACTGGTTTTACAGCTGTTTCAGCAGAAA
CGTAAGTTTTGGTCCCAGTTACTCTGTGATGGCTGGAAGCAGAAGTCTTCACTGGTTTTTCTCATTATTATCCATGTCTTGCTTTAGTAACAGA
TTTATCTTCTGATACTTAAAATATTGGTTGGTGTTGATCCATAATAAGTGCCAACAAATGGTACCAGTGATATGAGAAGTACAGAGAGACAAAC
TTTTGCAGGAGTTAAAGGTTAAGGTAAACTTCAGTCCAGTCTCTATCTGAATTACCATTCTCTTCTTCTGGACAGTGAGAAAAAGGAAAGGAAC
AGAACTACCATCTATTCCATACCTGTTATTTTCCAGGCTTTCTTCAAGTTACCTTAGTATATTATTTGCTATAATCATTAAATCAATAAAATAA
TAATTATTACAGCACCAACCTGAGCCTTAGAGAAGTAAAATAATAAAACTATGTACCTTGTAAGCTGCTGGAGCTAGAAGTTACAAACCCATCT
TTCTCCTTCCCACCATGCTGCTGCTCTTGCCTTATCATGAGATTACCAGCATTTATTGAGCACTTATGTTGCCTGTTCCACTGTAATTTCTGGC
CCATGGAGCCCATTTAAATTGCTTTTGCTTAGTCCCTTGTTCGTTAGAGATGCGCCAAGCTGTCTGATTTCTCCCTATCTTCGTTTTTTTTTTT
GAGACAGAGTCTTGCTGGGTTTCCCAGGCTGGAGTGCAGTGGCGTGATCTCGGCCCACTGCAACCTCTGCCTCCTGGGTTCAAGTGATTCTCCT
GCCTCAGCCTCCCAACTAACTGGGACCACAGGCACATGCCACCACGCCCGGCTAATTTTTTTGTATTTTTAGTACAGACTGTGGTTTTCACCAT
GCTGGCCAGGCTGGTTTTGAACTGCTGACCTCATGTGATCTACCCGCCTTGGCCTCCCAAAGTGCTGGAATTACAGGCATGACCCATTGCACCC
AGCCTCCCTACCTTCAGTTCTTAGGGGAGACAGGAGAGTCCTGAGCCATTGGATGGGGATGGTCTTCCGGAGGACAAGGTGCTGGGCAGAGTCT
AAGGGCACAGAGAAGCTTCACTCAACCTGAATGACCAATACACAACCGGCCAACCCACAGCCATAAGTACAGCCAGCAGCCCTGATCAGCCTCC
GTGTGGGCTTCCATTGACCGAGAGGCCACAGACTCCTGCTTTCCTGCCTCTTCCTGTCCCTGTCATCTCCCCGTCCACTTCCTTTATCCAGCAT
GTGATATTTCCCCCCAAAATCTAATCTCTTAGAGCAGAGAACTTGTTGCAAAGTAGGCAATGGAATTATTTTTCTGATTAGACCTGTGCCACTT
TTCCTTTTAATAGTTTGATGTTTGACCACTGCTTCTGGTGCCTTTAGTGAGATACCAGAGTCCATTTTTCAGGCTGAGGGAGGGTAAGGTCTTC
ATAATATAAGAAGGTATTTGGTGAACTCAGAGGTGGGCAGGTGGGTGAGAAATACACATTTCCTATTTGCCAGACATTGAGTGAAGCACTTTCT
GTATATTTCTCAAAAAATGGCGCACAGTCATTAATTATAGAAAGTCTAACAACTCGTATATCCTTAAAAGTATCTTGCACAGACATATTTTGCC
CCTCAATTAAATGTACGTAACTTTGCTTCTTCCTTTCTTCACATTCCTTGACCCTCCTCTCTCCATACTTGCTTGACATGAGGCATATTCGGAA
TAAAACCTTGTTAATTTCTACTCATCTAGTTTGAAATACATTGTGCTTTGGAGAGGAGCTTGGTTGTAGTTAATTTTTGAGCCAGCTCTGATAC
AAAGCTTTCTAAAGAACAATGGTAGCAGCCTTAGTTGCTTTCTCACAAAAAGTACGGCATACCATCTTTTAAATAAAGTGAAGAAATATGTGAT
TAAGTCATTATAGGATTAAATATGTGAGTACTTGCCTGGGGCCTCTTGAGTTCCTAATAGGCTTCTTTTGCTTTGCAGAGCTCTCTGACGATGA
CCCGCCAGAGATCCCACACGCCACATTCAAAGCCATGGCCTACAAGGAAGGAACCATGTTGAACTGTGAATGCAAGAGAGGTTTCCGCAGAATA
AAAAGCGGGTCACTCTATATGCTCTGTACAGGAAACTCTAGCCACTCGTCCTGGGACAACCAATGTCAATGCACAAGCTCTGGTAAGTGTCCCT
TCTGTCACTACCAAGAACAAAAGAACTGCAGCAAAGAGTGAGACAGAGCCCAGCTTTTGTTCACTCAGCCCTATAGACACAAATCCGCCAGTCC
AGTCTACACCATAACTAATTTTAGTGGTTCCTCAAAAGTCATTCACTAAACACAATTTTTTTTTTTTTTCCAGACAGACTCTCACTCTGTTGCC
TAGGCTGGAGTCCAATGGCACGCGATCTCGGCTCACTGCAACTTCCGCCTCCTGGCTTCAAGTGATTCTCCTGCCTCACCCTCCTCAGTAGCTG
GGATTACAGGCACCCTCCACCATGCCTGGCTAATTTTTGCATTTTTAGTAGAGATGGGGTTTCACCATGTTGGCCAGGCTGGTCTCAAACTCCT
GACCTTGAGTAATCTGCCCATCTCAGCCACCCAAAATCCTCGGATTACACGCATGAGTCACAGTGCCCGGCCACACAATTTTATTTTTATTTTT
GAGACAGTCTCGCTCTGTTGCCCAGGCTGGGGTGCGGTGGTACAATCTCAGATCACTGCAACCTCCACCTCCTGGGTTCAAGTGAATCTTGTGC
CACAGCTGCCCAAGTAGCTGGGACTACAGGTGTGCACCACCATGCCTGGCTAATTTTTTTATTTTTAGCAAAGATGGGGTTTCGCCATGTTGGC
CAGGCTGGTCTCGAACTCCTGGCCTCAAGTGATTCTCCTGCCTGGGCCTTATAGGCGCACACCACCATGCCTGCCTAATATTTTGTATTTTTTG
TTAGTGATGGGGTTTCCCCATGTTGTCCAGGCTGGTCTTAAACTCCTGGGCTCAAGCAGTCCTCCTGCCTCAGCCTCCCAATGTGCTGGAATTA
TAGGTGTGAGCCACCACACCTGACCTAGACACCCTCTTTTATCCTGAGCTGGAATACTGATCACCCCCCTCACTGGGGCACTCAGACTCAACTC
CTTTGCCAAACTCTTCACAATAGAGAGGGCTGTTCTAGAAGTTTCCTTGTCATTGTGTTAAGATGGCGTCTCTAACTTCAAGCATTCTGAACAG
CCTTAGGGAGCAAAGGATCAGTGTGTTGTGGAGTGAGACCACTCCTGCTGCTGTCAAACAACACAGAGACAATTGACAGCAAGCAAGGAGTCCC
TTTTGGTGAGAGACACACACATGCACACATAACATACATGACATACACAGACACGTAACATACATGGCATACACAGACACGTAACATACATGAC
ATGCACAGACACGTATACACACGCTCAAAAACATGTCGTGGGCATTGCCATATGCACTTGAATACCCAGAGGCCGGTGCTGGGAGGGCATACTC
TGGTTTCGTATTTGTAAGCTCAGCAGCCAGACACCACCACACACATCGTGGGCCCTGGACAGCTGTGCTTCTCAAGTGAATGAATACATCAACA
GTGTTTTCTATGAAGCATTTGTGAAATTATCTGCATCATTCCATCTATATTTTCTAGCCACTCGGAACACAACGAAACAAGTGACACCTCAACC
TGAAGAACAGAAAGAAAGCAAAACCACAGAAATGCAAAGTCCAATGCAGCCACTGCACCAAGCGAGCCTTCCAGGTGAGAGATGATTCTGTCCT
CCAGCTAACTCCTGCTAGCGCACCCTTCTCCTGCAGGCAGATGATGTGTACCCAAAGGAAGAAGGTCTCCCCTGCCTTCTATAAACACATGCAT
GTGGCTCCGTCCTCCCAAGACATCATTGCAGACAAGAATTAGTGCAGAGTTGCTCAGAGGCTGTTTGCTCAGCAACTAAAGCCAATCTGACGGC
AGCCAATCCAGCTCTCGGCTTACTCCCTGGTTTGGTAACTGGCCAAATAGAATTCAACCCAAATTCAACTCAACTTAAAAGCCTCTCTTGGGTC
TGTGTGCCAGGCCCTGCTGATAGAAGACACAAACAAGAGTGTGACAACGATCCCTGCCCTCAAGGAAACGCTCTGCGTCTTCCCCTTAAGCACA
AGACAACGCACCGCAAAAAAATTACTTGCTTCCCAGTATTTCTTCCAGGGCAAGGTTGTAAAATGTGCATGCCACACTTGTGAGTCTCAAAAGC
TCCAGATGCGTGTGTCCTGGCCAGCAGTCTGTGCATGAGTGGGCACTGTGGCTGTCCCTTGTCTCCCCTCTGCTTTCCCATGACTGAATTGGGA
TTCAGTTATGGTGTCAAGAAAATGGGCTGGGCGCAGTCGCTCACGCCTGTAATCCCAGCACTGTGGGAGTCTCAGGCAGGCAGATCACTTGAGG
TCAGGAGTTCGAGACCAGCCTGGCCAACATGGTGAAACCTTGTCTCTACAATCATACAAAAATTAAGCTGGGCATGGTGCTGGATGCCTGTAGT
CCCAGCTACTCAGGAGGCCAAGGCATGAGAATTGCTTGAAATCAGGAGGTGGAGCTTCCAGTGAGCAGAGATTGCATCACTGCACTCCAGCCTG
AGTGACAGAGGAAGACTCTGTCCCCCCTCAAAAAAACAAAACAAAAAAGAAAAAAAACCCACAGAACCTGCTAGGAAGAATTGAGCTTTCTAAG
GTCAGGCTAGCCTTAGGAGACTTCAGAGCCCTTTGCTGATTTTACAACCTTACAAAGAACTGTATCAGATACCATCCCATTTGAGCCTGGCTAT
ACAATGAGTCAGGACCAGGAAGGGTTACTTCAACTTACTGAAGAAACTGAGATGCACAAAAGATAAAAAGATTTCGTTAAGCTCACATAGTAGG
GCTGGAATCAGACCTCAGGTCTCCCAGATTAGATGAGGAAACTTTCTGCTGATTACTTATTTTAAAAGAGTTTGAATTTACTTACAGCTTCCAT
TATTTTATTTTTTAAAACAGACTAATCTAGGGGCACAGTTGCAACATGATGCTCTTACTCCTTCCCCTTCCGACTTTGTCCTGGAATGGTGTTT
CCAAGCACATTTGTTTGTTTTGTTCTACTGGCTCCCCCATAGTCACCCTGCACACATTATTGGAAGGCTTCAAAGAGGCCAATGCTTGTTGTCC
TTTCTCGAAGGCTGGCTCTGCCTGAGGGATGTTTCAAAGATTGTCTCAGTTTGGGTGTTTTCATTGGAAAGAAGCAAACCCAATTCAAACATGT
TCAGACACTTACGAATTGTACTGGCTGTCAAACAACCTTTTATCCTGAGACCAAAACACAAGTTCAGTCGTGATGTTCAAGGTGCCACATGGCA
GGAGCCAGCCTGGGACAGGGCCACCCAGCTTCCTGCTGGGGAAGAGGCTCTGGAGGATATTTACAGAGGCTCTGTTGGGTATGCAGGGTTGGGG
AGAGTCTGGAAGTTCAGAGCACGTGCGCAGTTTCACAGGCTCGGCTTAGACGGCACATACAATTCCATATGTCCAGGTTGGGGGCTGTGGGTGG
GGAATTCAGAGATTTAATCAGAACAAGACATGGTCTCACCCCACAGGTCCGTCAGGCCTGGGATGTAGTTGGGTGAGTGGAAAGTTCTGGGGAT
GGGAAATTTTGAGACGTCTTGAAAGGGCGCATCTGTGTCAACCTTCAGGTGTGGCTTGATCAGAGATGGTGCTCCATTTCTCTCAGCTTCCCTC
AGCCCGTCCCTCCTCCTGGCTTTGGTGTTGTCCCCAATCCTTCCGACATCATCCCAAGATGCCCAGCCGCTGCCCCCAAGAATGCACTTCCTCA
CTTGTCCAGCAGAAATCCAAGGTGGTCTTCATGCTCCATTGGCCCAAATAGGCTTGACCCTACCCTTGTGTCTCAGCTGAGACTGCCCCGCCCA
CTCCTGTCCACTGTCAACACCTGCACTGTCTTCCAAGAGGCAGTTGTGGCGTTCCTGTCTCGGGGCCGCACCAACCAGAATGAATCGCTCATTC
CTCTGTGTTCCCATTGCCAACGTCTGCTATCCTTCACATGCCTTTCTTACCCAATCCACTGTCCACCCCTTGCAGGAGTCACAGGGTCTTCGTG
CCTTCCTACAGGGCCTAGCAAACTTCCCCACGCTCAGACGTTCTCAACATATATTGGTTGAATGAAAGGATGAGCAAGCAGTCAAGAGAGTAAG
AACCAAGGTCCAGTCCCTAGCAAGAGGCAACCTGGACTCACTCGCGGTGCTGTCCCCAACTTGCCTTCAGAGCGTTCCTTCCATCTTCCAGGTC
ACTGCAGGGAACCTCCACCATGGGAAAATGAAGCCACAGACAGAATTTATCATTTCGTGGTGGGGCAGATGGTTTATTATCAGTGCGTCCAGGG
ATACAGGGCTCTACACAGAGGTCCTGCTGAGAGCGTCTGCAAAATGACCCACGGGAAGACAAGGTGGACCCAGCCCCAGCTCATATGCACAGGT
GAAATGGAGACCAGTCAGTTTCCAGGTACGGTCGCTCCCTTCTGGGGTCCACAATGTTGCTTTGCTCCATCAGGCTGACCAGGGTGGAATCCCT
GACCACTCCTGCTGGACAAGAGTGGTCCTTGGACAAGACCCTTGATCTCCCTGGGCTTCCATTTTTTCTCATTCTGAGAAAAAAAAAAAATTTA
AATAGACCAGATCAGATGGACTCTAAGGCCTCCTTCAATGAAGCTCCCCCTGTGCCCTGAGTGGGGAGAAAGGTTGGAGACAGAAGAGAAGGAA
AGCCCTCTCCTAAGCACCCTAGATGCTGCCTCGTAGTTGGTCCTAAGTCCACTACTTCTCTAAGCCACCAAGGCCTCCTCTGGCTCCAAGGCCA
AGTCCAGTTTCCCACTCACACATTTTTCCTCTTACTGGTGATCGTGTAAATGCATGATCTGAACAGCCCTCCGCGGTCAGCATGTTCCTTCCCC
TTGTGCCATTCCCCACCTCAGCTTGTAAAAATAAACATAAAATGTGCAGAGTCGGGACTTACTCAGTGCACATGCGCGCACACACACACACACA
CACACATACTCATGCAAATGCTCAGCCCACCGATGTCGAGGTCTCCTGCTGTCTTCCTTCTGAAATCTTATTAGTATCACGACAATCAAAAGTG
ACTTACATGCACGCTGGGCACAGTGGCTCACACCTGTAATCCCAGCACTCTGGGAGGCCAAGGCGGGCAGATCACTTTAGGTCAGGAGTTCGAG
ACCAGCCTGGCCAACATGGTGAAACCCCGTCTCTACTAAAAATACAAAAATTATCTGGGCGTGGTGGTGGGTGCCTGTAATCCCAGCTACTTGG
GAGGCTGAGGCAGGAGAATCACTTGAACCCAGGAGGTGGAGTTGCACTGAGCTGAGATCACACCACTGCACTCCAGCCTGGGCAACAGCAAGAC
TCCGTCTCAAAAAAAAAAAGTGACTTACATTCAACCACAAGTATTCCTGGTTTTCTTGCTCACTGCACTTCTCATACTCCATGTCCTTCTCTTG
GGTTCCCTAGGGAGGCTGTTTTGTTCCCTTAGCTCCTTCCCTGGGCCACCCTGACAGCCAGGGAATGCTTGCTGCTTCCTTGGAAGAGGGTGAG
AGGCAGGGCAGAGCCAGGGTTCCTCACCCCTCAGCAGAAGTGGAGAGCCTGGCACAGAAGCAGCTGTCCCTTAGTGCATCTCTGCTCCATCCAG
TGGTTCAATGCCATTCGTATTATTATGACCTGTTTCCAAGTCACAGATGGAAACCAGGACTGGGCAGGGAGGCTGGGGAGAAAGCATTGCTCAG
AGTTTTGGGGACAGCTAGGGTCAGAGTTAGAATTCAGCCTCGTCTGCTCTGCTGAGACCACTTCACCGCTTCCCAGTGCCCCTGCTGTGGAACC
ATGTGGGGTGCCATCCCTGGCGAAGTGCCCACACCAGCATTCCTGCCTCCTCGGGCGTGTCTGGGGCCGGCTGCCTGGGGCAGGCGGGGTGGGA
GTGAGGCCCTGACTCCTGTGTTTAGCTCCACCAGCATCACTTACTCTCTCCCCCAGGTGAAGAGAAGCCTCAGGCAAGCCCCGAAGGCCGTCCT
GAGAGTGAGACTTCCTGCCTCGTCACAACAACAGGTGCGGGAGAAGACAAACGCTGGACCACAGAGGCCTAGTCCAAAAGGGCAGGGGTGACCA
GGAGCCAGGCTCAGGGAGATAGGCCGAGGTGACCTGTAGGGGAGAAGCCCACAGCAGCCTCCTCTCCCTCTCAGCAGGGACAGGGCCTCAGCAA
CCTGCAGGCCCCAAAGCAAGTGTCAGAAAGAGGGAACCCAGGACCAGGACCAAGCACGGTCCCCAGGCAGAGATGGAACACCTTCCCTCACCAC
CACCACGTGTCTCCCACCAGCCTCTGAGCTTCTCATTCACAGAGACACCCTGACTTCCTTTAGCCTCGTGCTGTCCTAAAGTCACGGTAGCAGG
ACTGTCTCTCTTTATCTCTTTTTCACAGATTTTCAAATACAGACAGAAATGGCTGCAACCATGGAGACGTCCATATTTACAACAGAGTACCAGG
TAGCAGGTGAGTGGGGCACTGGCTTTGTGGACAAAATGTACACCAGGCTGAGATATGGACAGGTTGACTGGTTAGTAGGTTGGTTAGTTGGTTG
GCTGGTAGGTTGGTTACTTGGTTGATTGGTAGGTTGATAGGTAGGTTGGCTAGTTGATTGGTAAGTTGGTTCGTAGGTTGGGAGGTTAGTTGGT
TAGTTGGTTGGTTGCTTGGTTGGCAGGTTGGTTTGTATGTTGGTAGGTTACTTGATTGGTTGGTAGGTTGGTAGATTGGCAGGTTGGTGGGTTA
GTTGGTTAGTTGGTTGGTAGGTTGGTTGGCAACTTCATAGGTTGGTTGGTGGGTTGGTTGATAAGTTGATAGGTTGGTTGGTAAGTTGATAGGT
TGGTTGGTGGGTTGGTTGATAAGTTGATAGGTTGGTTGCTACGTTGGTTGTTAGGGTGGTAGATTGGCAGGTTGGTTGGTAGTTTTGTTGGTAG
TTTGGTTGGTTAGCTGGTTGGTTGGTTAGTGACTTGGTTGGTAGATAGATTGGTAGATTGGTTGGTTGGTTAGTTTGTTGGTAGGTTAGTAGGT
TTGTTAGTTGGTTAGTTGATTGGTAGGTTGGTTGGTTGACTGAAAGGACCCCTTCCTTACACTCCACAATCTGTGACTGAAAGACTAATCCAGA
TACTGACGTAACTCAGAATTCGCATGAACCAGTTACAGGATGCTGTGAGCAAACTCTCAAGTCAGCCCTATCAGCTCCTCTCAAGCTGATCTGG
AAGGGCTGTACCCCGGGCCTCTGCCAGGGGTCAAGAGTGGGAGGAAGGAGCCACGTAGCCAGTCAGAGAGGACAGGCTTCCAGAAACACTGCCA
TTGTTTCCTGGTAGGAGGGAAAAACACACTCTGAGTCTGCATCTGCCTAGAAAGTAAAGAAATGTCAGCCTCCAAAACCTCAGCAGAGCAGCAT
CTGGAAAACCCGACCTCTTTCAGGAGATGTTAATGTGCTTCTCATCATCTTCATATTTCAACATTTCTTTTACCAGCCGCTCTCGGTGTCCTTA
AACGAGGCCTGGCCTCTGAGCCTTTCTGAGGGGCTTTTGTGAGAGAAGGGTGCCTCCTGAGGGGAAGCTCCCTCCACCCTCACCCTGTCCTCCC
TGCCCTGTTCCTCAAGACTCCTAGGAAATCCCATGGGCAGCCACCTACTTCCTGAGACAGGGAAGACAGGCTCCAGCCTCCGCCTTGCTCTTTG
GGGATGTCCTCAGCCTGCCCTGTGGCCCTGGCTCCTGCTGCCCCCATGCCAAGCCCAGGGCCCTTTGCTCAACTCCCTCTCTCTATTGACAGTG
GCCGGCTGTGTTTTCCTGCTGATCAGCGTCCTCCTCCTGAGTGGGCTCACCTGGCAGCCCAGACAGTAAGTGTGGCATCACCAAGGCAGCCCTT
GGTCAGATCAAAGTCCTGTACCCAGCCCCACCCTGCCTCCCCCCTACCCCCTCCATGCTCTCTCTAATCACCTGCAGTTCAGGGAGTGGCAAAG
GGAGAGGCCAATCCATGCCTCGGAAAGAGTCAGTTAAAAAGAATTAAAGCACCGCCGGGTGCAGTGGCTCACGCTGGTAATCACAACACTTTGG
GAGGCCAAGGCTGAGTCGATCACCTGAGGTCAGGAGTTTGAGACAAGCCTGGCCAACATGGCAAAACCCCATCTCTACTAAAAATACAAAAAAT
CAGTTGGGCATGGTGGCAGGTGCCTGTAATCCCAGCTACTTGGGCGGCTGAGGCAGGAAGATTGCTTGAACCTGGGAGGCGGAGGTTGCAGTGA
ACAGAAATCTCACCACTGCACTCCAGCCTGGGTGACAGAGCAAGACTCTGTCTCAAAAAAAAAAAAAAAAAAAAATTAAACGACCAGGGAAATG
GGAAGGCAAAGGAGCCTCACATTCATTGAGCACCCACTATACACCAGGCTTGATGCTTGTTTTATAAACATTATTGAAATCTACCTTCTCAGAA
ACCTTTGGAATAGCTGTGGCCTAGAGAGATGCAACAGCTTGGAGAAGATGCCACAGCAGAGGCCAGCACTGCAACATGAATCTAGAGCCCATGA
TTTTCCCACCCAGCCCCTACCTATAGAGGACCCAGGGCAGCATCTATGCTGAGCTTTGCCTATCCTCTGCTCCCCACCCTCCCCTCCACACAAG
CTCACACCGGCATCCCCTTGACCAGGAATGTAAACCATGTGTGGTCTTTTTGCATTAACTCAGATTTTCAACCCTGCATGCATCTTAGAACATC
CAGGAGACAGTCTTCTAAAATACCACTGAGTCGCAAGATATGAGTAAATTCCACACGAAAAGGAAGGCAGTCCAAAGGGCTGTGCTTCTCCTGT
GGCTCAGTGTTATTCATAGCTGTGACATCTTTTAAGAGAGGTTTACAAGAGGGAAATCCTGTCACCAAAACCATTGTGACCCACTTTGAATTTG
GGGGGAATCTTCATTTGCAACAATAGAAGAACAAATTTCTTGCAATAGGAAATATTTTTGTAGCTTATGTAGGTTGTTCAGCCAAAACACCATA
ATAATTTAAAAAGTTAAAAGATAGCCCAGGAGTTGTGGCTCATGCCTGTAATCCCAGCACTTTGGGAGGCTGAGGCAGGTGGTTACCTGAGGTC
AGGAGTTCAGGACCAGGCTGGACAACATGGTGAAACCCTGTTTCTACTAAAAAATACAAAAATAAGCCAGGCGTGGTGGTGGGTGCCTGTAATC
CCAGCTACTTGCGAGGCTGAGACAGGAGAATCACTTGAACCCAGGAGGCGGAGGTTGCAGTGAGCCAAGATCGCGCCACTACACTCCAGCCTCG
GCAACAAGAGTGAAACTCTGTCTCAAAAAAAAAAAAGAAAAAGGTAAAAGATATCAATGATTGGGCTCCAGTCAGAATGTCTGGGGGTGAGCCC
AAGACATCAGTACGTATTTTTAATTGAGATAAAATTCATCATTTAACCATCATAAAATGTACAGTTCAGTGGTGTCTTAGCCCATTTTGTGCTA
CTGTAACAGAATGCCTGAGACTGGGTAACTTATAAAGAACAGAGATTTATAAAGAACAGAGATTTATAACTTATAAAGAACAGAGATTTCTTAC
CATTCTGGAGGCTGGGAAGTCTGAGGTCGAGGGGCCCCCATGTATCAAGCGCGTTTGTGCTACGTCATCCTGTGGCAGAAGGCAGAAGGGCAAG
AGAGTGAGAGACAGCAGGAGGGGCTGAACTCAGCTTTTATCACAAACCCACTCCTGCAATAACAACATTAATCCATTCATGAGGCAGAGCCCTC
GCGACCTGATCACCTCTTCAAGATCTCACCTGTCAACATTGCTGGATTAGGGATTAAGTTTCCAACACCTGAGCTCTGGGGGATGTATTCAAAC
CACAGTAAGTCGTATTTAGTATGTTTGCAATGTTGTACAGCCATCCCCACTGATTCCAGAATACTTCCATCATTCCCAAGAGAAGCCCCACACT
GCCAAGTTCCCCATTCTCCCCAGCCCCGGGAAACCACCACTCTATGTTCCGTCTCTATGGATTTGCCTATTCTGGACATTTCGTCTAAATGGAA
TCACGTGGCCTTTTATGTCTGGGATAGTCGTACGTTTTAAAGCCCCTCAGATGTATCTAATACATATGCATCCAGGTTTGACAACCCTACACCC
GGCCATTGGTTTTCAAACTTGAACTAGCACCAGAATCATCTGTAGGAACATAAAGAAACGATAAATGTTTGAGATGATGGATATGCTAAATACC
ATGATCTGATCACTGTACCTTGTAGGTATCAAAGCATCCCTACATACCTCATAAATATATATAACTATTATGTGTCAACTTAAAAACATTTGTT
GAAAGAATCATCTGGAGGCCTTGTTAAAGCACAGATTGCTGGGTCCCACCTGTGGAGTTTCTGACGCAGTCGTCTTGCTTAGGTCCAGGAATCT
GTATTTCTATCAGGTTCCCAGGTGATACTGACGCTGCCGTTCCAGGGAGCAGACTCTGAGGATCACCGCACTAGGCTAACAGCATTAAGAATCA
GGGAAGCTGGCCAGACTCACGCTTGTAATCCCAGCACTTTGGGAGGCCGAGGCAGGCGGATTACTTGAGGTCAGGAGTTTGAGACCAGCCTGGC
CAACATGGAGAAACCCTGTCTCTTCTAAAAATACAAAAATTAGCTGGCTGTCATGGTGCGCACCTATAATCTTAGCTACTCGGGAGGCTGATGC
AGGAGAATCGCTTGAACCCAGGAGGCAGAGGCTGCAGTGAGCTGAGATCGTACCACTGCACTCCACCCTGGGTGACACAGCAAGACTCTGTTTA
AAAAAAAAAAGAAAGAAAGAAAAAAGAAAAGAAAGAAAAAGAAAAAGTAAACTCTAGAAACTCCCTCTGTCTCTCTCTTGATTATCCAGGGCCT
CCCTGACCCCTGCCAGCATCCACTCTGGCCAACCCTGATACAAACTCCACAGTAAGACCCTCAGAAAATACAGATCCACTCCCCCAAAACCAAA
GCCCTTAGCCCAGCCAGAGACAAAAATAAAATATCTCTTCCCTTCTTATATGCGTCATCAGGTCCATAACATACTCCTAATTTTCCTCATGGGA
ATTAAAAAGTATAAATTACTGCAGTGCAAATGATAAGCCCCCTAGGGTTATTGTAGCACGAATGATTCATATACCCGGCAGTCTACCAGTTTCT
CTAAGGGGTTAGCTGACTCCTAAGGTGTCATTTTAGGCTTCCAGGAGGGGAAGGATGTGACGTGGCTCACATGTTGTACGTGTACTCAGTACTT
GCATCCTCTCCTTCCTCAGGCTGAACACCTGACAAGCCTTAACACCTTGCAAGGATTTTTTTCATTTGTTAGTTTGAAGATCAAAAGCCTTCGG
GCCCAGAGGACAAAGCTATTTGGAAGTAGGAATGGTTCAGTGTTTATGTTGTGAAACAAATAACTGTGTGAACCTATGTGAGTTCCCACAAGGT
CTCCCAGAACTACAATCTTTGGGGAGACTCAAACATACATCTAGAAATGACCCCTCTAGTCAGTTTGTCCAAAAGCCTTCCTAGAATCTGATAT
CAACCCATGGTGCCCTCTTGTGGCAACAGAAATGCTGCATTTGAATTACCGAAAGATCACTGTAGCCCAGCCAGAGAGTTTTGGTTGGGGAAAT
ATTTTTTGTTTGTTTCGGGCAATGACCAGGAGCCCGTGATGTGCTACAGAAGGCTCACAGTGGCTTGTGAGAGCCAGCGGTTAAATTTTCGGGA
CTTCTGCAAACTGGTTTTTAAGCATAGCCATTATGGAAAATTAAAGTATTTAGGCTGGGAGCAGTAGCTCCTGCCTGTAATTCCTGCACTTCGG
GAGACTAAGGTGAGAGGCTCGCTTGAGACCAGCCTGGGCAACAAAGTGAGACCTTGTCTCTACAAAAACATTTTAAACTTAGCTAGGCGAGGTG
GCACATACCTGTAGTCCCAGCTACTGGTAAGGGCGAGGTGGGAGAATCCCTTGAGTCCAGCAGTTCAAGATTGCAGTGAGCTGTGACCATGCCA
CTGCACTGCAGCCTGGGTGACAGATTGAGACGCTGTCTCAAAAAAAAAAAAAAAAAATTAAAATATTTAAATTTACAATTTAATAAATTATATT
AAAAACAAAGGCAAGAATTACTTACAACTGATCACTTCCTAATTATTTGCTACCTTTTACTGTTATCTTTGTTCGCTGGGTGGTTTACATCTAT
TGTGTCCGCATGGTGGAAATACCGTTTGACGGTGGCTACCGCGCATCTCTTCCCAACCGCGTGATCAGTGATGTCACATTGCCAGCTGGAAGCT
GTGTGTGGAGGGAGGATTGACACCAGGGAAGACAGGCAACCGTCATCAGAGAGCTGCTTGTTAAACATCACCAGCACACCTCTGCTTGGAGTCT
TGTGCTATGGGAACTCTGTGCAAAGTAGGGAGTAATTAGAAATTCCCATGAGCCAAATAAGAACTGGGAAAAGGCTAAGACCGTATTGTCCAAT
ACAGTAGCCACTAGCCACCTGTGGATACTTAAGTGTATTTAAACTCAAATAAATTAAATACAATTAAAAATTCAGTTCCTCGGTTGCACTAGCC
ACATTTCAAGCGTTCCCTAGCCACAGGTGTCTGTGTTATCGGACAGCACAACTGCAAAAGATTTCCATCATCTCAGAAAGTTCTATAGCACAGT
GCTGGCTAGAAACCAAGTGCTTTACTGCATGCACATCATTTAGCACAGTTAGTTGCTGTTTATTATTCCTGTTCCACAGCTATTGTCTGCCATA
TAAAAACTTAGGCCAGGCACAGTGGCTCACACCTGTAATCCCAGCACTTTGGAAGGCCGAGGCAGGCAGATCACAAGGTCAGGAGTTCGAGACC
AGCCTGGCCAACATAGCAAAACCCCATCTCTACTAAAAATACAAAAATTAGCCAGGCATGGTGGCGTGTGCACTGGTTTACAGTGAGGACCACA
TTTTTTTGGTGCCGTGTTACACATATGACCGTCACTTTGTTACACCACTACAGGAGGAAGAGTAGAAGAACAATCTAGAAAACCAAAAGAACAA
GAATTTCTTGGTAAGAAGCCGGGAACAGACAACAGAAGTCATGAAGCCCAAGTGAAATCAAAGGTGCTAAATGGTCGCCCAGGAGACATCCGTT
GTGCTTGCCTGCGTTTTGGAAGCTCTGAAGTCACATCACAGGACACGGGGCAGTGGCAACCTTCTCTCTATGCCAGCTCAGTCCCATCAGAGAG
CGAGCGCTACCCACTTCTAAATAGCAATTTCGCCGTTGAAGAGGAAGGGCAAAACCACTAGAACTCTCCATCTTATTTTCATGTATATGTGTTC
ATTAAAGCATGAATGGTATGGAACTCTCTCCACCCTATATGTAGTATAAAGAAAAGTAGGTTTACATTCATCTCATTCCAACTTCCCAGTTCAG
GAGTCCCAAGGAAAGCCCCAGCACTAACGTAAATACACAACACACACACTCTACCCTATACAACTGGACATTGTCTGCGTGGTTCCTTTCTCAG
CCGCTTCTGACTGCTGATTCTCCCGTTCACGTTGCCTAATAAACATCCTTCAAGAACTCTGGGCTGCTACCCAGAAATCATTTTACCCTTGGCT
CAATCCTCTAAGCTAACCCCCTTCTACTCAGCCTTCAGTCTTGAATTTCTAAAAAACAGAGGCCATGGCAGAATAATCTTTGGGTAACTTCAAA
ACAGGGCAGCCAAACCCATGAGGCAATGTCAGGAACAGAACGATGAATGAGGTCCCAGGCAGAGAATCATACTTAGCAAAGTTTTACCTGTGCG
TTACTAATTGGCCTCTTTAAGAGTTAGTTTCTTTGGGATTGCTATGAATGATACCCTGAATTTGGCCTGCACTAATTTGATGTTTACAGGTGGA
CACACAAGGTGCAAATCAATGCGTACGTTTCCTGAGAAGTCTCTAAAAACACCAAAAAGGGATCCGTACATTCAATGTTTATGCAAGGAAGGAA
AGAAAGAAGGAAGTGAAGAGGGAGAAGGGATGGAGGTCACACTGGTAGAACGTAACCACGGAAAAGAGCGCATGAGCCCTGGCACGGTGGCTCA
GGCCTATAACCCCAGCTCCCTAGGAGACCAAGGCGGGAGCATCTCTTGAGGCCAGGAGTTTGAGACCAGCCTGGGCAGCATAGCAAGACACATC
CCTACAAAAAATTAGAAATTGGCTGGATGTGGTGGCATACGCCTGTAGTCCTAGCCACTCAGGAGGCTGAGGCAGGAGGATTGCTTGAGCCCAG
GAGTTCGAGGCTGCAGTCAGTCATGATGGCACCACTGCACTCCAGCCTGGGCAACAGAGCAAGATCCTGTCTTTAAGGAAAAAAAGACAAGATG
AGCATACCAGCAGTCCTTGAACATTATCAAAAAGTTCAGCATATTAGAATCACCGGGAGGCCTTGTTAAAAGAGTTCGCTGGGCCCATCTTCAG
AGTCTCTGAGTTGTTGGTCTGGAATAGAGCCAAACGTTTTGTGTGTCTAACAATTCCCAGGTGCTGTTGCTGCTGCTACTATTCCAGGAACACA
CTTTGAGAACCATTGTTATTGCTCTGCACGCCCACCCACTCTCAACTCCCACGAAAAAAATCAACTTCCAGAGCTAAGATTTCGGTGGAAGTCC
TGGTTCCATATCTGGTGCAAGATCTCCCCTCACGAATCAGTTGAGTCAACATTCTAGCTCAACAACATCACACGATTAACATTAACGAAAATTA
TTCATTTGGGAAACTATCAGCCAGTTTTCACTTCTGAAGGGGCAGGAGAGTGTTATGAGAAATCACGGCAGTTTTCAGCACGGTCCAGATTCAG
ATTAAATAACTATTTTCTGTCATTTCTGTGACCAACCACATACAAACAGACTCATCTGTGCACTCTCCCCCTCCCCCTTCAGGTATATGTTTTC
TGAGTAAAGTTGAAAAGAATCTCAGACCAGAAAATATAGATATATATTTAAATCTTACTTGAGTAGAACTGATTACCACTTTTGGGTGTTGAGG
GGTCTATAAGATCAAAACTTTTCCATGATAATACTAAGATGTTATCGACCATTTATCTGTCCTTCTCTCAAAAGTGTATGGTGGAATTTTCCAG
AAGCTATGTGATACGTGATGATGTCATCACTCTGCTGTTAACATATAATAAATTTATTCCTATTGTTTATAAAACAATAAATGATATTTTTTAA
AAAGTCTTGGTTTTTATTATAAATATGATAAATATTGCTACCTATAATCTACATAACCAAAAACCTTTATAGTTTTTTGGTGCTTTTTTTTTGT
TTTTTTGTTTTTTTTTTTTTTGAGATGGAGTGTTGCTCCGTTGCCCAGGCTGGAGTGCAGTGCACAATCTCGGCTCACTGCAGCTTCTGCCTGC
TGGGTTCAAGTGATTCTCTTGCCTCAGCCTCCCAAGTAGCTGGGACTACAGGCACCCACCACCAACTCCAGCTAATTTTTGTATTTTTACCAGA
GACGGCGTTTCACCATGTTGGCCAGGCTGGTCTCGAACTCCTCACCTCAAGTCATCCACCCAACTCAGCCTCCCAAAGTGGTGGGATTACAGGC
GTGAGCCACGGCACCCAGCCTGGTATCCTCTATACTTTTTAATAGTTTTAAATAACATGCAAAGATCCTGTGAAATGGCCAACAAGCACATCAA
CATCACTGGTCATTTCAGAAATGCGAATCAAAACCACAATGAGATACTACTTCATACCCATTAAGATGACTTTAAGAAAAAGAATAGGAATGCT
CACACATGGCTGGTAGGAATCTCAAAATGTGCAGCCACTGTGCAAAACACACTCTGTTTTGGCAGTTCCTCAAAAAGCTAAACATAAATTTGTC
CTATAACCCAGCAATTGTATTCCTAGGTACACACCCAAGAAAGACAAAAACGTGTCTAAATACAAACTTGTACACGACTCTTTATAACAGCATT
ATTCATACTCACCAAGAGGTAGAAACACCCAAATACCCAACAACAAAGGGATGGATAAAGGCCGGGCATGGTCGCTCATGCCTGTAATCCCAGC
ACTTTCGGATGCCAAGGTGGCCACATCATTTGAGGCCAGGAGTTGGAGACCACCCTGCCCAACATGGTGAAACCTCATCTCCACTGTCTCTACT
AAAAATACAAAAAATTAGCTGGTCATAGTGGCGCGCATCTATAATCCCAGCTGCGTGGGAGGCTGAGCCACCAGAATCCCTTGAACCTGGGAGG
CAGAGGTTGCAGTGAGCCAAGATGGTGCCACTGCACTCCAGCCTGGGCGACAGAGGGACTCTTCTCTCCAAAAAAAAAAAAAAAAAAGAATTGA
TAAGCAAATCATGGCATATCCCTCCCACTGGAGTATTAGCCACAGGAAGGGATGAAGTATTGATCCATGCTGCCATTTAGATGAACCTCAAAAA
CATTATGCTGAGTGAAGGAAGCCAGACACAAAAGATCACTACTGTATGACTCATTTAACATGAAATATCTAGAATAGCCAATTCCATGGAGACA
GAAAGCAGATAAGTGTTTGCCAGGGGATTTTTCTGGGGTGGTAAAAAAGTTTTGAAACTAGAGGGAGGAGGTAGTTGCACAACATGTTGAATAC
ACGAACAGCCACTGGATTGTATACTTTATCATGGTTAATTGTGTGTTATGTGAATTTCGCCTCAACTTTTTAAAAAGGAGATCCTGAGGCCAAA
ATATTTAAATATTGCTTATCTTATTCTTGATGCTCAAAGAAGGGTCATCAGCCCAGAAGGATCCTCCTGAAGTCTTTATGGTGACTTTAGTATT
CTACTAAGTGAGTTATCAGGGGACCCTGAATTTTTGTTTTCAAAAACCAAAATTAGCATATTACTAAAGTTTATCGAAGACTCCACTTTGGTAT
TCTATCGAGAACGTAAAATTTGCATAGCTCCCAGGAAGGCTGCTGTTCCTGATGCATCACCAGAACTATCGTGAGTTGGTTTCAGTTTTTACTC
GCTGGTATTCCTTCAGCTAAATGATGAATTCCTGCCAGTGATTTTCATGGAGGGGTTGTGTTGGCTGCCTCCCAACGTGCATAATCAATTAATT
TGCTGTGGGTTGAAAATCTGAACAAACTGAAAGTCAATAACTCTTCTTAGGTCTGTAGAAGTAAGTAAGATTACAGGGTAGCTCACTGTCCCCA
ATATTGGAGAGGCAGACAGGCTGATACAGGGAAATCATAACCTCCTGGACCAGAAACCCAGGAACAAACCCTGCTACTGGAACCAGCAGAGGGG
CAGCAAAACCTAAACTGGAACTGACAAATTGCTGGAGGCTCAGTGTGGACACTTCTGAGAGATAAAAATTTCAGGGGAGCAACCCAGTCACAGG
GGGTCCCCACTTTCATGACCTTTCCCTCCAGGAACTCAATGAGGTCTTCACAGTGAAGATGGCAGAAAAACTTCCTTGTGCTTTTGACGGGGGA
AAGAGAAAAGGTACTGTCACACGCATCCGTGTGAAGAGAGTCCACCAACAGGCTTTGTGTGAGCAACAAGCCTGTTTATTTCACCTAGGTGCGG
GCGGGCTGAGTCCGAAAAAGGATTCAGCAAAGGGTGGTGGGATTATCATTAGTTCTTATAGATTTCCCATAGGCATACAAAGTACATTCACAAG
GGTGGGGAGAATATTACAAAGTACCTTCTTAAGGTCAGGGCAGAACATATCGTATCAGTTAGGGTGGGGCAGGAACAAATCACAATGGTGGAAT
GTCATCAGTTAAGGCTATTTTCACTTCTTTTGTCCATCTTCAGTTGCTTCAGGCCATCTGGATGTATACGTGCAGCTCACAGCGGATATGATGG
CTTAACTTGGGCTCAGAGGCCTGACAGGTACCATTCTCAAATATGCCAGAGCATTCAGTACTTGTCCAACAAGGCCTGCCCTCAAGAAACAGTA
TTTTCCTCACTATCTTAAATTTTACCATAGTCTAACCAACCTAAATTTTACCAGTGTCTAACCAACCTGAAAGAAGGGAAATAACCAGCTCTAG
CCTCTCTGCCCTTCTTGTCTCATCTAAGCGAGGACGCGAATGAAAAGCACTTGTGAAGGTCACAGCCCAGGGGTACAGGCTTGCTGAACGCTGA
ACCTAATCATAAGACTATGGAATGGTTCCCCTCCCCCTGCACCTTACCACCACATCAGTGGGGCTGTTGTGCGCTCACAGGCGACTACAGTGGA
AACAACTGCACATTTCAGTCCCTCTTTAAGAAGCAGTCTCTAGGGAAGCCCAAAGATGAGAGGGGAGACAAAAACAAAGACACTAGAGGGAATT
TTAGCCTCTGACATCTACAGCTACAGCAAATAGCAAACACACCCTAACTCCTAACCAGATAAACATAAAGGCTCACACTAAAGGCCTATTTACC
TTGGTTCCTTTTATGCTGTATATCAGGTCCAGCTTTCAACAAAAAATTACAAGGCATGCTAAATGGCAAAAAAACACAGATTAAAGAGGCAAAG
CAAGCATTGTAACCAGACTCGGATACTACCATGTTGGAATTATCAGACTGGGAATTTAAAACAACTACACAAATAAGCCATTATGACAGAGATT
TCTATGGTCCTGTAGATCCATTCTTCCCTTCCACCATCAGTAATAAAATCCCCAAATTTTAACCCAACACATTTCTGCCCCTAGCTAAAGACAA
CACTTTCTAGCCTCCCTTGCAGCTAAGTGTGGTCATGTGACTAAATTTGGATGAGATGTAAACCAAAAAGTATCTGAGACAGGTCTAAATCCAT
TCAGAGGTTTATATTGCCAACATTAAACACCATGACCTGTGATACAGCATCAGGAGTTCCTGAGAACATGTGACCAAGGTGGTTGAGTTACAGC
TGGGTTTGATATTAATGCATTTTAAGGAGACAGAAATTATAGGCGAAGACATAAAGCAATACATGTAAGGTATACATTGGTTCAGCCTAGAGAG
GTGGGACATGTCAAAGTGGGAGGCTTCCAGGTCATAGTTGATTCAAAAGTTTTCCGATTGGCAATTGGTTGAAAGAGTTAAGCTATGCCTGAGG
AGTTGAAGTCAGTAAAAAGAAATGCTTGAGTTAAGATAAGGGGAGTGGGGTTGTGGAAGACACTGTCCTTGTCATGTACACGAAGCTTCCAGAT
AGCAGGCTTCAGAGAGAATAGAGAGCACATATATCTTATTGCAACTTAACAGACGTCAGACTCTCTGGAAAAGACCCATTAAGAGAAGGGAGAT
TCCTCCAGTATGAAAATTAAGAGACACCTTTGCAGGGCCACTTCAAAATATGTCAAAAAATATTTTGGGGTAAAATAATTTTATTTCCTTCTTG
GCCTGTTATATGTCATGTGATGCTATACCAGAATCAAGCTGGGATTGGGTATCTTGTTGCTAAAGCATCTGTTTCTTAGGTCTTATGATCTCTA
TTTTAATGTAATCCTGGTCAGTTGATGCCTGAATTCCAAAGGAAGGGATTACAATGAGGCGTGTCAACCCCCCCACTTCTTGTCATGACCTGAA
CTAGTTTTTCAGTTTCTTGGGGATGCCCTTGGCCAAAAAGGGGCTCATTTAGTCAGTTGGGGGCTTACAATTTTATGTTTGGTTTACAGAGATA
TGAGCAGAAGGGACGTCTGTGACGCCCAGGGTGTGCCCTTGAAGAGAATGTGCACACCCTCCTTCATCTCCTCTCACTGGTGGCTGCGTGCTCT
TGCTCTCGTGAGCTAGAACCACCGGGAGGATAGAAGCCACACATGAAGCCTGAGAGGGAAAAAAAAATCCAAGGAGGCTGGGAGTCTGACCCCA
TGAGGTGCCCCAGCAGCAGTCCACACACTCGGACTGCATATCAGACAGAAATAACCCTCCCATTTGCTGCGATTTTTGCCCTTGTTAGGGCAGC
ATCCTAACTGTATCAATCACCCACACTCCTACTAAAATGACACCCTCGGCTATCGTTTGGATATGGTTTTGTTACCACAAAGGGGTCCTGCTCC
AGACCACAAGAGAGGGTTCTTGGATCTCATGCAAAAAACAATTCAGGACGAGTCTGTAAAGTAAAGTGAAACAAGTTTATTAGGAAAGTAAAGG
GATAAAAGAATGGCTACTCCATAGACAGAGCAGTTCCGAGGGCTGCTGGTTACCTATTTTTATGGTTATTCCTTGATGATACGCTAAACAAGGG
GTGGATTATTCATGCCTCCCCTTTTGAGACCATATAGGGTAACTTCCCGATGTTGCCATGGCATTTGTAAACTGTCATGGCAGCGGTGGGAGTG
TAGCACTGACGACAACCAGAGGTCACTCTTGTCTCTATTTTGGTTTTCGTCGCTTTTAGCCGGCTTCTTTACTCCAACCTGTTTTATCAGCAAG
GTCTTTATGACCTGTATCTTGTGCCAAACTCCTGTCTCATCTTGTGACTTAGAATCCCTTAACCATGTGGGAATGCAGCCCAGTAGGTCTCAGC
CTTAGTCTATCCAGCCCCTATTCAAGATGGAGTTGCTCTGGTTCAAACACCTCTAACAGTTCTTTATCCCAAGCAAATCTCATGTTGGACTGTG
ATTCCCAGTGTTGGAGGTAGACCTAATAAGAGGTGTTTGTGTCATGAGGGTGGATCCTTCATGAATAGATGATTGCCCTCCCTGGGTGAGGGGG
GTGAGTGAGTGCTCATTCTATTCATTCCCGTGAGAGCTGGTTGTTAAAAAGAGCCTGGCATCTCCCCCCACCCTTTCTTCCTCTCTCACCATGT
GACCTCTGCACACAACAGCCCCCCTTCACCTCCCACCAGGAGCAGAAGCAGCCTAAGACCTTTCCCAGTGTAGATGCCCAATCTTGAACTTTCC
AGACACCAGAACTGTGAGGCAAATAAAACTTTTTCTTTATAAATGATTCAGTCTCCAGCGGGGCATGGTGGCGCACACCTGTAATCCCAGCTAC
TCGGAGGCTGAGGCAGGAGAATCACTTGAACCCACGAGTTGGAGGTTGCAGTGAGCAGAGATCGCGCCACTGCACTCCATCCTGGCCACAGAGC
GAGACTCTGTCAAAAAAAAAAAAAAAGAGAGAGAGAGAGGGAGAGAGAGAGAAAGGAAGGAAGGAAGGAAGGAAGGAAGGAAGGAAGGAAGGAA
GGAAGGAAAAAAGCAAAGAAAGAAAGAAAGAAAGGAAAGAAAGAAAGCAAAGAAAGAAACAAAGAAAGAAAAAGAAAGAAAGAAAGAAAGAAAA
GGAAGGAAGGAAGAAAGAAAGAAAGAAAGAAAGAAAGAAAGAAAGAAAGAAAGAAAGAAAGAAAGACCAGTCTCAGGTATTCTCAGGTTACAGC
AACACAAAACAGACTAAGACACCCTCTCCACCAAAATTCTGTTTGTATAGCATATTAGTATCAAAATCTTTTCAATGGAAAATGGACTCCAGAA
TCAATATTCAACATGACAGCAGAGTTTCAAGTAAATACATGTTTTCTGTTCCATAAATATTATCTGAAAAAGATTTCCCTACAGGAGATAAACA
AGTAGATAACATAAAATAACATTCGTTCTCTTTATTAATTTCAATACTCCCCTACTTTTCTTCAGAAAATCACTGCATTCATTCTCCAACACCC
CTGAAGCCTTTCTGAAATGTTGGCTCACATGGAGATGTTCCTTTTGCCTGATGTCTTCTGGCTAAGTCAGAAACTGCAAAGGGAAACCTGCTGA
AGTTTTAAAAACTTTTCCCTGTTATTTCCGTTTCTCCTCCTAAAGAGTGCAGTGCGAAACACGTGTCCTCTTCTTCTACGATGTTGATGAACAT
ACCTTCAGAGCTAGCCTATTGATATGAAGGTAAGGATGAAAAGAAATCTGTTATCTCAGGGCAGTAGGATTATCAAGCACCATATCAGCAAATG
CATCACTCGAAACTAAATTGTAAATATAGCCATGCACCGCATAACAATGTTTTCATCAACAACAGACTGCATATACACGACAGTGGTTCCATAA
GATTATAATACCATATTTTTACCATACCTTTTCCATGTTTACACACACAAATACCCCCAGGCGCAGTGGTTCACATCTGCAATCCCAGCACTTT
GGGAGGCCAAGGCGGGTGGATCATCTGCGGTCAGAAGTTCCACACCAGCCTGACCAACATGGTGAAACCCCGACTCTACTAAAAATACAAAGTT
AGCCAGGTGTGGTGGCGCATGCTTGTAATCCCATCTACTTGAGAGGCTGAGGCAGGAGAATCGCTCGAACCTGGGAAGCGGAGGTTGCAGTGAG
CCAAGATTACACCGTTGCACTCCAGCCTGGGCAACAGAGTGAGACTCTCTCTCAAACAAACAAACAAACAAACAAAAAGATACACAAATATATA
CCTTAGTATCACAATTGCCTGCAGTATTCAGTATAGTAACCTGCTTTACAGGTTTGTAGCTACGGACGACACGCTATAGCAGCCCCAGAGTGCA
GTAGGCTCTACCATCTAGGTTTGTGTAAGTTCACTCTGCGACGTTTGCACAAAAACAAAATCACCTAACAATGCATTTCTCAGAAAGTATCCCC
ATCATTAAGTGACTCGTGACTGTAATAGAAAATGAAAACCCACTGTCTCTCATAACTGTGTACCCTCCAGTGGTGCTTCCTGCATGGCTCAGCA
ACCTTATCAAGAACCTGGTTATGGGTATTTTTTCTTCTCTCTTTTCTGTCTCCAATGATGTCAAATTTATTCAAAGACCAGCTTAGCCTCGTGA
TAGCCAACAGGTTGTAGCTATTCCAGAATTAATATCAGATTATCACATCTAGAAATAAGAAAAGACATAAGGTTCCCTCGTTGGAGCTTCCAGC
AAATGTCTTCTCCAGTCTTGTTGACTCAAACTAGGTCTCATATCTACTCCTGCACTGTGACCAGAAGATGTGAGATGATTCTGATTTCGTAAGG
CCCAAGCCACAGACCTCACTCCTAGAACCAGTAATAAAGAGAAGATCTTAAAAGCAGCCAGAGAAAAACGAACCTTTGCAGAGAAATATAAATA
AGGACGACAGCCAGACCCGATGGCTCATGCCTATAATCCCAGCACTTTGGGAGGTCGAGGCAGGTGGATCACTCGAGGTCAGGAGTTGAGACTA
GCCTGGCCAACATGGTGAAATCTTGTCTTTACTAAAAATACAAAAATTACCCAGGCATCGTGGCACATCCCTGTAATCCCAACTACTCAGCAGA
CTGAGGCAGGAGAATCACTTGAACCCAAGAGGTGGAGGTTGCAGTGAGCCAAGATCGCACCACTGCACTCCACCCTGGGTGGCAGAGTGAGATT
CTGTCTCAAAATAAATAAATAAATGCAGATTTCTCTCTGGTAACAGTATAAGTGGAGCAATATCTTTAAACTACTGAAAGAAAAAACTGTCAAC
CTAAAGTTCTATATGCAGGGAAAATAGCCTTCAAAAATGAAGATGAAATAAGTATTTTTAGACATATAAAAGTTGAAAGACTTCATAACCAGCA
GGCTTACATGCCAATAAATATTAAAGGACTCGGCTAAAATTTCACTGGGTTGAATAATGTCTCCCCAGAATTCATGCCTACTTGGAACCGACAA
ATGTGGCCTTGTTTGAAAATACAATCTTTGGAAATGTAACAAGTTAAGATGAAGTCATACCACATTAGGCTGGACCTTAAACCCAATATGACTG
GTGTTCTCATAAGAACAGCGAAATCTGCACGCAGACCCACACACAGGGAAGACAGCCAGGTGAAGACAGAGTCAGAGATTGGAGGATTTTTTTT
TTTTTTGACACAGACTCTCACTCTCTCACTTAGGCTGGAGTACGGTGGCACCATCTCTGCTCACTGCAGCCTCTGCCTCCCGGGCTGAAGAGAT
TCTCATGCCTCAGCCGCCCAAGTTGCTGAGATTAGACGTGTATGCCACGACACCTGGCTAATTTTTGTATTTTTTTGAGTAGAGACCGGGTTTT
GCCATATTTCTCAGGCTGGTTTCAGACTCCCGGTCTCAAGTAATCCACTTGCCTTGGCCTCCCAAAGTGCTGGGTTAGAGAAGTGAGCCACTGC
GCCCCGCTGGAGAGGTTGGAGCTTGATGCCACGAGCTGAGGAACACCAAGGATTGTCAGCAATAATCAGGTTATACACTGTTTGACTCCATTTA
TATAAATTTCTAGAAAATCCCAACTGCCCTATACTGACACAAAGCAGATCAAACACTGTCAGGGGAAGATGACAAAGGAACATTATCTTGATTG
TGGTTTTAGGGGTGTGTACTCATGCCAAAACTTACCAAACTGTCTACTTCGAATATGTGCAGTTTTTCAATGTTCACTAAACCCCCAATAAAAC
TCTTTCTCAAAAAATTACCTGTTTGAAATTCCT
HUMAN SEQUENCE—mRNA
GAGAGACTGGATGCACCCACAAGGGTGACAGCCCAGCCGCACCGATCTTCCCATCCCACATCCTCCGGCGCGATGCCAAAAAGAGGCTCACGGC
AACTGGGCCTTCTGCAGAGAAAGACCTCCGCTTCACTGCCCCGGCTGGTCCCAAGGGTCAGGAAGATGGATTCATACCTGCTGATGTGGGGACT
GCTCACGTTCATCATGGTGCCTCGCTGCCAGGCAGAGCTCTGTGACGATGACCCGCCAGAGATCCCACACGCCACATTCAAAGCCATCGCCTAC
AAGGAAGCAACCATGTTGAACTGTGAATGCAAGAGAGGTTTCCGCAGAATAAAAAGCGGGTCACTCTATATGCTCTGTACAGGAAACTCTAGCC
ACTCGTCCTGCCACAACCAATGTCAATCCACAAGCTCTGCCACTCGGAACACAACGAAACAAGTGACACCTCAACCTGAAGAACAGAAAGAAAG
GAAAACCACAGAAATGCAAAGTCCAATGCAGCCAGTGGACCAAGCGAGCCTTCCAGGTCACTGCAGGGAACCTCCACCATGGGAAAATCAAGCC
ACAGAGAGAATTTATCATTTCGTGGTGGGGCAGATGGTTTATTATCAGTGCGTCCAGGGATACAGGGCTCTACACAGAGGTCCTGCTGAGAGCG
TCTGCAAAATGACCCACGGGAAGACAAGGTGGACCCAGCCCCAGCTCATATGCACAGGTGAAATGGAGACCAGTCAGTTTCCAGGTGAAGAGAA
GCCTCAGGCAAGCCCCGAAGGCCGTCCTGAGAGTGAGACTTCCTGCCTCGTCACAACAACAGATTTTCAAATACAGACAGAAATGGCTGCAACC
ATGGAGACGTCCATATTTACAACAGAGTACCAGGTAGCAGTGGCCGGCTGTGTTTTCCTGCTGATCAGCGTCCTCCTCCTGAGTGGGCTCACCT
GGCAGCGGAGACAGAGGAAGAGTAGAAGAACAATCTAGAAAACCAAAAGAACAAGAATTTCTTGGTAAGAAGCCGGGAACAGACAACAGAAGTC
ATGAAGCCCAAGTGAAATCAAAGGTGCTAAATGGTCGCCCAGGAGACATCCGTTGTGCTTGCCTGCGTTTTGGAAGCTCTGAAGTCACATCACA
GGACACGGGGCAGTGGCAACCTTGTCTCTATGCCAGCTCAGTCCCATCAGAGAGCGAGCGCTACCCACTTCTAAATAGCAATTTCGCCGTTGAA
GAGGAAGGGCAAAACCACTAGAACTCTCCATCTTATTTTCATGTATATGTGTTCATTAAAGCATGAATGGTATGGAACTCTCTCCACCCTATAT
GTAGTATAAAGAAAAGTAGGTTTACATTCATCTCATTCCAACTTCCCAGTTCAGGAGTCCCAAGGAAAGCCCCAGCACTAACGTAAATACACAA
CACACACACTCTACCCTATACAACTGGACATTGTCTGCGTGGTTCCTTTCTCAGCCGCTTCTGACTGCTGATTCTCCCGTTCACGTTGCCTAAT
AAACATCCTTCAAGAACTCTGGGCTGCTACCCAGAAATCATTTTACCCTTGGCTCAATCCTCTAAGCTAACCCCCTTCTACTGAGCCTTCAGTC
TTGAATTTCTAAAAAACAGAGGCCATGGCAGAATAATCTTTGGGTAACTTCAAAACGGGGCAGCCAAACCCATGAGGCAATGTCAGGAACAGAA
GGATGAATGAGGTCCCAGGCAGAGAATCATACTTAGCAAAGTTTTACCTGTGCGTTACTAATTGGCCTCTTTAAGAGTTAGTTTCTTTGGGATT
GCTATGAATGATACCCTGAATTTGGCCTGCACTAATTTGATGTTTACAGGTGGACACACAAGGTGCAAATCAATGCGTACGTTTCCTGAGAAGT
GTCTAAAAACACCAAAAAGGGATCCGTACATTCAATGTTTATGCAAGGAAGGAAAGAAAGAAGGAAGTGAAGAGGGAGAAGGGATGGAGGTCAC
ACTGGTAGAACGTAACCACGGAAAAGAGCGCATCAGGCCTGGCACGGTGGCTCAGGCCTATAACCCCAGCTCCCTAGGAGACCAAGGCGGGAGC
ATCTCTTGAGGCCAGGAGTTTGAGACCAGCCTGGGCAGCATAGCAAGACACATCCCTACAAAAAATTAGAAATTGGCTGGATGTGGTGGCATAC
GCCTGTAGTCCTAGCCACTCAGGAGGCTGAGGCAGGAGGATTGCTTGAGCCCAGGAGTTCGAGGCTGCAGTCAGTCATGATGGCACCACTGCAC
TCCAGCCTGGGCAACAGAGCAAGATCCTGTCTTTAAGGAAAAAAAGACAAGG
HUMAN SEQUENCE—CODING
ATGGATTCATACCTGCTGATGTGGGGACTGCTCACGTTCATCATGGTGCCTGGCTGCCAGGCAGAGCTCTGTGACGATGACCCGCCAGAGATCC
CACACGCCACATTCAAAGCCATGGCCTACAAGGAAGGAACCATGTTGAACTGTGAATGCAAGAGAGGTTTCCGCAGAATAAAAAGCGGGTCACT
CTATATGCTCTGTACAGGAAACTCTAGCCACTCGTCCTGGGACAACCAATGTCAATGCACAAGCTCTGCCACTCGGAACACAACGAAACAAGTG
ACACCTCAACCTGAAGAACAGAAAGAAAGGAAAACCACAGAAATGCAAAGTCCAATGCAGCCAGTGGACCAAGCGAGCCTTCCAGGTCACTGCA
GGGAACCTCCACCATGGGAAAATGAAGCCACAGAGAGAATTTATCATTTCGTGGTGGGGCAGATGGTTTATTATCAGTGCGTCCAGGGATACAG
GGCTCTACACAGAGGTCCTGCTGAGAGCGTCTGCAAAATGACCCACGGGAAGACAAGGTGGACCCAGCCCCAGCTCATATGCACAGGTGAAATG
GAGACCAGTCAGTTTCCAGGTGAAGAGAAGCCTCAGGCAAGCCCCGAAGGCCGTCCTGAGAGTGAGACTTCCTGCCTCGTCACAACAACAGATT
TTCAAATACAGACAGAAATGGCTGCAACCATGGAGACGTCCATATTTACAACAGAGTACCAGGTAGCAGTGGCCGGCTGTGTTTTCCTGCTGAT
CAGCGTCCTCCTCCTGAGTGGGCTCACCTGGCAGCGGAGACAGAGGAAGAGTAGAAGAACAATCTAG

TABLE 20

MOUSE NOMENCLATURE
ICSGNM Nfkb1
Celera mCG22374
HUMAN NOMENCLATURE
HGNC NFKB1
Celera hCG39336
MOUSE SEQUENCE—GENOMIC
TCTTCTGAATATCATGTTTAGATTATGTAAGTATTCACCGGCTTTCTGTCACTATAAACCAATACCTCAGTTGATTAATCAATAAAGAAAAAAA
GGTTTAGTTGATGAGTTTTGAAGGTTCCAGTCTACCAGTCTGTTACTGCAGACGTCCACCGTCATAGTATGTCATGGCCAGAGCACCTGGCAAA
GCATAAACTACCTGCTCGTCAATCAGGAAGCAAATAGAAAAGAGAACTGGTTGCGGTCCCACAACATCCTTCCAGGTCATGTCTTAGTAGAGCA
CTTCTCCCAGGAGAAGCACCCTCTTGCCCTTGCCGTCAACACATGAACAGCTTGGGGACTCCTACTGAGGTAACAGTACCCATTCTGCATACAT
TCCCTGAGATTAACTGAGACTTGATTCATGGCCCGCTGTTTTGTCTGCCTTTGTGAGTGTTCTGCTTGCAGCCCTACTAAACACAGATGCCCAA
TGTGGTATTCAGAGTAGTGGGCCTTTAAGAGGTTATGAGGTCATAGCACATACCCTGCCCCTGCCATGAATAGGATTTTAAAGGGGGCTGATGA
AGGAGGGTTGTCCCCTCTGACTCTTCCACCTTTACTGTGTGACAATACAGTGATTGTTTCCTCGTGACATTCCTCATTTGCGTGCTTTGACCTT
GCACTTCTTGCCTCCAGAGTGATAAAAAATAAACTTATGTTTTTTATAAACTACCCAGACTCAAGTACTGTTTAACAGTACAGACATATGAAGA
CAGTTGGTCCTGTTTGTAGTCACCACCGAAAGAGGAAAGTTGAAAACTTCCACTCTACTTGTGGATTTCTGTCTCTTCCACAAATGTTTAGGTC
TACTACTTTGTACTTCATGTATTTTGGAGACCTATTAATAAGTGCATGCAGGTGGAGGTCATTATTTTTATAAGGGCTCAGTAGTGCATACATA
TAAGGTTCCAACACTCAGCATCTAGAGGCAGGAGGGTTGCTGGGAGTTTGAGCCCAGCCAGAGCTATCTATAGAGACCCTGTCATAAAACAACA
AAATTATTACTGCCCATTGATGACCTCGCCCCTTTATTCTTATATATATATCCTTCTTTGCCCTTAGTAATATTCCACCCCCTGATCTTCTCTA
TTGGTGGTTATGATATAAACTACTTTGCTGTGTTTAGTATTTGTATTATTAGCACAGTATCCCAGGCTTCTGCTTTTAAACACTTTGCGTCTTT
CATGTTAAAAATAATTCATAGGAAAAGCATTTTCTTGCGTCTTGCTTTTTCTGTACTGTTTACCATGAATGCTTTTTATAGCTGAGTTTATTTT
ATTTGCATTTAATATTCGACCTCTTCTTTATCCACATTTTGTCTTTCATTGTCATCTTTTAAATGAAATAAACGTACCACTTGAAGAGTTCCAT
TTTATCTCCCGTCTGGTAGTTCTGTAAAACGGTGGCTTTCTGTACTACGACTTTCTGCATCCTGCGTCTATAGCGAGTGAAGCGCTATGTACCT
AATGTGCAAGCTTTGATTTGTTCTCACCTCTCTGCTAGTGCTGGTAGACTTTTTTCCCTCCATGGGTTCAGAATACTCCATAATATATTGTGTT
GTTCAATGTGTTTGTTGTACAGTAAGTTTGAACATACTGCAAAGCGAAAGCCCCATGTGCCCACACCTTTATCACCAGTAGTGTTTTCCTTGTT
TCTTAAAGGTCCAATTCTATCTCATTTCCCTTCACCACAAAGATCTGCTTTTAGGATGTCTTCTGTCACTTCTACGCGTTATGAATTCTCTCAG
CTCTGCTTTATTAGAAAGTGTCAGTTTACCTTTATTGTCAAACCATTCTGGGACAAAGCTACCTGCTTCTTTGTGGCTTCAATGTTTTTGTTGT
TCATTATAGATATCATGATGTGAAGAGTTTGCATGTTTTTCTTTTATTCAGAGTTGAGTTTTGTCCTAGTAGGCCTTACTAACATGCATTTAAA
GTCGCCCTTATTTTATGATGTGTTCTTGACTCTTATAACACGGTTTTCCCCTAGACTGCTATAAGCACACATCTTCAGAACGTTTACTGATGTC
TCTTCAGCCCACGTGATTGCACCCTCAGTGTCTCTCAGCTCTGTATGCCGTCTCTAATCTTATCAGAGCATTTATAGAACAACAGTGGCTTTCA
CTGAGCTTTTTGGGATCTCTGTTAGGATTTCATCAGAGTAAAGGTGGGCTGATTATAGATTTTTGAGGCTTTGCACATATACCATTTCTCTGAC
CAATGAGATCACTGCTCTTTATTTGAACTCTGCTTCCTGTGGCCTGGATTAGAAAGTGCCCTCAGTCAGAAGGTGAGCTTGGAGTTCACTTAAA
CTGTGCAGCCTGTATCTCTTGTTTAGTGTCTCAGGCTATTGTGTTTCATAATTACATATGAATTTGCCAAGATCAAATAGTGGTTGATTTATAA
TGACTGAAATTAGATTTGTGTGGATTTTTAAAGATTTTTTTTTTCAACACAGGGTTTCTCTGTGTAGCCCTGGCTGTCCTGGAACCCACTCTGT
AGACCAGGCTGATCTTGAACTCAGATCTACCTGCCTCTGCCTCTGGAGTGCTAGGATTAAAGGCATGCTCCACCACTGCCCAGCTGTAGCATGC
TATTAATGATTTTGAAAGACATTATTCCAGGGCATTTATATATTCAATATGTTGGCTAGCTAGCATTTCTCTACAGCTTTGTTTTGAAACTTTC
TCATCATCCCAGAAGAGTGCCTTGAATGTGGTAAATGATGGCTCCCTTCCCAGTCTTCACTCACTCCCCTGGCCTCTGGCAAGCAATGATGTGT
TCCTCTTCCTACGAATGTGCTTGTTCTTGGTATTCAATGTGAAGGGAATTGAGTATAGGTGAGCGACCCTTTCTGTCTGGTTTCAGTATAAATC
ATTTAGTCTACTGGTTTTGAAATACAAAATCAAAACTCCCATTCCTGGCTGTAAGGCACACCATTTAAGGAGACTGTAAATATATAATGTATGA
TTAAGTTGCATAGAAACAGTGAACCATAGTGCAAAACCACCAGGAAATGTTATAGACCTTATCGACTGAGGCGATAACAGTGGAACAGAAAATA
CAGGTTTTATGGAAGAAGTTGATTTTGAAGTACCATGTTGTGGAATACATAATATCTAAACAGGATAGAATCTCGAGTGTGAAGAGATGTACAC
ACTGGAAAGAAATGACATAGAGCTGTTACATGTGTGTCTGTACACATCCGTGTGTGCGTGTATGCAGTGAGCATTCACATGTGTAGAGGTCAAA
GGACAATTTTGGAAGCTGCTTCTTTCCTTCGAGCATGTAAGTTCCAAGTAAGGACTTAGGTCCTTGGGCTTAGCAGCAAGTGTTTTTACCTGCA
GAGCCCTCTTGTTGGCCAAGCAATGCATCTTTAAATGATTGATTTCCCTATAGTAAAGTGCTTGTGTTGGATCATGGTGATAGTTGTGGTGCTT
ATATAATATATAATATAATATAATCATATGCTGAGTAGTTGCTCTTTGGACTTCACTATGTAAAGCACCTGGCTGTGATCTGAAAGATATTTAA
TCAGGAAGATACCCTCCCATGCGGGTGAGCGGTAGAAAAGCTGTTTCTGTCAGAGTGAGGTAATAAGGTCTGCCCTACCGTGTGGCTATGGACA
TAGAAAGGAAGTGCACACACAAGAAACAGTGTGAGGAAAGAAGTGAGCGACAACAAGACTGTTCACTGCATTGGTTGGAGAAGGCTACATTTGC
AAGAATGACACAGTCAGGAGTCCGGCTTGTTCTCCAGTAGTAAACTGTAGACATTAGGGTTGATGGGTTTAATGTATCATTAACTTGTTTTCTA
ATGGCATCGTTACCTAAAGCTAACAGGGGCTCATTACCAAATACACCTTCAGAGCCTTAACAGTTCTAATCATGTACTGATGTCACGGGGGTTG
GGCTCGTATCTTGGATAGCTTGGGTTTTTTGTTTTGTTTTGTTTCTTTTTGCTCTTTCCTTTTTTTAAAAAAAATTATTTATTTTTTGTTCAAT
TGAGAACTCAGTAGCCGATGGAGAAAAGCCACTGCAAAGTCCAGACCGTGACTAAGAGTGTAATTAAAGCTGTGATACTGAATTGCCAGGGTAA
TTAACCCATGCTCCACACGCCCTCTGTTCCCATGACACTGACCAAGCTGTATCGTGTAGGCTGCTGTCTTTCCATTTACTAACCCTTCTGATTG
GCTAGCCTTTGCTGCGGCGAGATAAATCATGAAGAAGCACATAGAAACACAGATATGCCTCATTTCTTGCGACCTGTGACTGTGTGTGGTGATT
TGTCTGGCTGCTCTGTGGAGGTCCTCAGCTGCTCTGCCACCGACGGCTCAGCCTGGGAAGTCTTGGCTGAGCTCTGCTGCGTTTCCACTCCACA
CGTGGGTTCTCATCCTCCACTGGGCTGGCCAGGCCTTTCCCTTCCAGGAAAGAAAATCCAGTCATTGAACGGCTTCTTGAAAGCCATGTTCATG
ACTCATGTCTGCATCCTCCTGTTGGTCAGGGCCTGCCAAGGGCCTGACAACATTCAAGGGGCAGAGATGTCAATTTTATTGTCCCTGTGTTGTG
AGGGATAGAAAAGGGCAGTGGCCATTGTTTTGAGCCTACTACAGCTATAAGCCCTGAGTTCAGACTATGCCACTCTTGTGTAGTTTTGTTTCTT
TGGAAACTCACAGGGTAGCCAAACCTCTTTCTGATGGGTTGATGAAGGGCCAAAAACATCCAGGTAACATTTCAAACATGGTTTCCCTGTGATA
CAGACCATGGCATACACAAGTATCACAAATCTGTTCCTACAGCACTGTGGATGACACACACACACACTCTCTCTCTCTTTCTCTCTCTCTCTCT
CTCTCTCTCTCTCTCTCACACACACACACACACACATGTTTTAATTTTTAGTTTAGCTAACTGTGGCTTTTGTGAGATATTCTCTCTCTCTCTC
TCTCTCTCTCTCTCTCTCTCTCTCTCTCTCTCTCTCTCTCTCTCTTTCTCTCTCTCTCTTTCTCTCTCTCCCTTCACGTTTGGATTGCCTTGTA
AGTAGCTCATCTGAAATTCATGCACTGTAAGGTCCTATTCTGTTCTCTGGGTTACTCTGTAGAAGAGTTCTATGGGCTCACCAGGTACTAGATG
TAGGAACAGAAAGCACACTCGGGAGAAGGCATGTTGCAGAAACGAGAGAAACATGCTGAGACTCCTAATGTGCATCTCCCCAGTGGAGACAACA
CAATTTGACTGGACAGTGTGTGCATGAAGGCTAAAGAGCGCTCAGGGAATATAGAGGGAGCCTCTTCCCGCTCCAGTCACCGTGACAGCATGCT
ACTTCGTGTGTTGGCATGCACAAAATTGGCAGAACGCCTCAAGTGTGGAGAATTCACCATTATAACAAGATTATAGTAAGCAAAAAATCACTCC
ACATCACGTAGAAGAGGGAGTTGGCAAAGGCGGCTGCTTTAAACCAGCTCTTTGCAATCTTCTCTAGACCGTCTTATCTGAAAGCAAACAAAAA
ACAACAGGAACAGCTTGAATGTTAGCAGTTATCCTGGCAGGCAGCACTCCAACCCAGCCCACCCCACCCAGCCCCCAGCACAGGTGTGTAACCC
TTTAGTAGCCTACTTAATATCGCTGAAGAGATAGATGGCTTAACTAGTAAGAGTGCTTACTGCTCTTGCAGACGACTCAGGCTGGTTCCTAACT
CCCACATGATGACTGACAACCTCCTGTCACGCTGGTTCCAGGGGATCTGGCACTCCCCTCCTTGCCTCTGCCAGTACCCTTACATTTGTGGTGC
ACAGTCATGCTTGCAGGCAAAACACTCAAGCCACATAAAATAAAAATTAATAAAACCTTGAAAAAAAGATCCTAGCTAGCAGTTAATATCATGA
GTAGACTAGCTCCAGCCAGAACTTTTGAGAGGCTTGAAGCAAAGATGTTTTTAAATGAATAATACTTCTGTCAAGAGGAAAATCACATCACCCA
TTACGCTAGTACAAGGGCCATAAGCTGTGCAGTTAGAGAGGCCAGCTCTAGTCTGTCAGCCTGAGCAAATGACTCATTCTCTCTGAAGCTTGGC
TCTTCAGGTGGACTCTACTTTCTGGGCTCAGCATAGCAAGAGGGCAGCATACGTAAGCATTACCTCCCCTTCCTTAGTTATGTGGAAGCACTCT
GTCAAGCTTAGCTTTCCCCATACAAAGGAGAGCACTTCTCCTGAGGCCAGAGCACTCACTGGCTTCTCCTCCTCAGCCTAACTGGCACCAGGGA
TTAGGTAAATTGTCCCTGGGTTAATCCTGAGCCTTCCAGGATCTTTGGATCACTTCTAGAAGATGGTAGGACCAGCCATCCAGTCCTGTGAGCC
CAGAATACTTCAGACAATAACTTCTAAACTCCCCCTCAGGGAAAAATACAAGTTATAGCGTAATGCCATGCAACCACTTCAGACTCAAATATGT
TAGACTGTCAGTAGCTATTGGCAGAAAATTTATCTGTCTTTGCTACAGTTCTATTGCTCTGAAGAGACATCATGACCAAGGCAACTCTTCTAAA
AGAAAGCCTTTAAATGGGCCTGGCGTACAATTGTAGACGTTTAGTCCATTATCATGATGGCAGAGAGCAGAGGACACAGGCAGGCACAGAATCA
GGAGCTGAGAGTTCTGCATCATCGTCTGTAGCCAGAGAAAGACAGACTCTGGACTTGGCATGGGCTTTTGAAACCTCAGTGCTCCTCCTCAGTG
ACAGACTTCCTCTAACAAGGCCACACTTCGTAATCCTTTTAATCCTTTCAAATAGTGTCACTCCCTGGTGACTAAGCACACAAATATATGAGCC
TGTGTCTTTCTCAGGACAACGAAAAGTTTATCTTGCATCTTTCTTCCTATAATTTATCATGAGTGAGCTATTAGACTGTCTGGTGAGTTAACAT
ATGTTCTGTATCTCCCACTCACTAATAACCTTCGCATGGATTAGCTAGCTGAGTGTTTCCCATGGGTCTGCCACCCCATTAGGCTGACACTCCA
TCATGAATGGGTTTCTTCGTCAGCTCTCAGATTTGCATTTGCAGTGCGTGCATGTATTGTTCTAGGTCATTGTCACGATGCTCCTGGTGAAATA
ATATTGAAAATAAAAGAACTCCGAAACCAGGTTCTGTAAATCTCAGTGTGGTAGCATGCACAGAAACATTGATTTATTTTTAAAAGAAAGCTTT
TAGAAATCAGCATTACTGAAAAAAAAAAAGTCCATTGCCAGCCTTTATCATTAGTGTGACATACTGTCAGACAGGAGGACACACAGAAAGGGGG
ACGCTTCTGAAGAGCAGTCAGGAATAATTAGGAGTGTTGACGAATTCATACTTCATATTTTATTTAACACTGTCAGGGCATTTTAGGTTCAATG
ATTTAAATTTAGATTCAGCCCCCTTTTTATTCTAATACATGTCTAATGTCTATTAGATAAATTTCCATGTCACTGTACAAATCTCCCCATTGTT
ATAGCAATTCCATAATATTTAACTTACAGTTGTACAACAAGAATGCAAACAGTTAGATCAGAGCACATCACAAACTCCTGAAAATCGACTTGCA
GAATTTACGCCCTCTGAATATTTTACATGGTATACACTTTATGTGGCCATAGCCCTTACATTATGTTTTCTTCAAAAAATTCTCTATGCAAAAG
AAACTTTAACATTTTATAATTTAATCTTTAGCAATTTATTGTGCGTCCAAGATACAAAACAAAAAATGTTGTGAAAAGTCTCTTTTCCCAGTAT
CTTTGCCCCATTGCTAAACAAAACAGTACATTAAATGACACCTAAGTGACACCTGTTATGGTTACTAGAATTTTAAACCACCCTGCCACCCCAC
CCCCTCCCTGATTTCCAAGTGCACCTTCTAGCTGGTGTCTGACCGCCTACTTTTCATAGTGCGCATTGTGGTTCTCTTTCGATTATGGTATTTT
AATGGGTTTGGAAATTTAGAGAGCCTTTTCCCCCAAAGAAATCGAACAGAACAGAAAGAGTTGTTGGTGTTGGTGGTGTTGTTGTTGGTGGTGC
TTACAGTGAGTCAGGCCTCGGCTGCCTATATCTCTGAACCTAGATGCTATATAGTTACTGACAGGAATTCTCCTGACTTTGTCCTTGATCGCAT
ACAGCCATGCCTTTAGAATTTCTCCACTCAGTCCAGCTGCTTCACCAAGATGCCCCTGTGTCAGATTGAGTTACTAGGAAGATTGCCACAGGAA
TGCAGGAAACAGTTCGTTCAGCATTCATAGAATATGTCACATACTCAGGACTTGGCTGGTTTTGTTACATTGTGCTTTGTATCAAAACCACAAC
TCCCCGGATATTTGCTGAGACCTAGCTACATTTTCTGCGTCTCCTTATGTGGTGAGAAGATCCCTAAAGATTCCTGGTTGGGGTAGACAAATGC
ATAAATGAGACCCAGGCTTGTCTCTCCCTTTGTGTCTCAAGGTCCTTCCTCAAAAGCGCAAAAGCAAACTGGGGGGAAGGCTGAGAAAGCAGGG
GTACATAAGGGACTTTCCCACTGCTTTCTCATCCCCAGCTCCAAAGCTTAGTCGATATTTCAACATTTTAGGTATAAACTTGCTTTTGCAATCA
ATGCAAATATTACTCAGTGATTCATCCTTTCCAACTGACTCTCGGTTTCTTTCTTTTGACATTTAATTTAAAATTAGAAATTAGCATTTATATC
ATGCTAGAGCTGCACTTACCTTTCAACGCCTCAGTAATGGCCAAGGCTTTATGCGTCAAGATTCTAAGGCACCCGAGTGTTCCTTGAGCTTCTG
TAGTACCTGACGGAGTTAAGTTTTTAGCCACATATAATCTAGAAACCGTAGGAGAGGGTGCCATGATGACAAGGTGCCCAGTATGGTCGTTTGT
CAGACATTTAAAGTCGAGCCGTCTTCATTAGGGCCTCATGTAGGGAATGGACCAGGAGGATACCCTTACCTCTGTCAAGTGCGGTTCGACTTGA
ATCTTCCAAGTTGGAGTGAGACTTCTTTATGTTAAATTCCCCCACTCAAACTTCTACAAATCATCCAATTTTAAAGAATGATTTTTAAGATGAT
AATATATTATATCATTTCCCCTTCCCCTGCCTCCAAGTTCTTTCATGTACCTCTCTCTCAAAACTTCATGACCTCTTTTTCTTTAATTGATGTA
TCTTTTTCTAAGTACAAAAAAAAAAAAATCAATCTGCTCCATCCATCTAATGTTACTTGTATGAATGTTTCCATTGGCATTGACTAACCAATCA
GTGTACATTTTTGGGAAACACAATTTCTCATGCTCTCAGCTTTCCCTATGTGCCTCTAGTTCTTAGTCTAGGGTTGAGACCTCCTGAGCAATCT
CCCTTTCACATTGCCTGTCTGTTGGTGCCATCCTTGTTCAGGTACACACAGAATCAATCACATAAAGGTACTAAGTGGGTTTCGACAGTTTAAT
AAGGCAATAATCTCACCTCCACCTTAGAAGATTGTCTTTGGATTGTTAGGAAAGAATATCATCACCCAGGTTAATGGAAAACCTAGAGTCATAA
GGATAGTGTAAAACATTGTTTGTGGAAATTAATTAGTATCCAAAGTCACATACCAGACAGTGGCAGAACCCTGGTATTTTCAATGATGTCATAC
CCCTTTACTGCATCAAGTCAAAATTCCCCTGTTTTAACTTTTTTGTCTGAATAAATATGTTTAGTTCCAGCTGCTTAGAGAGTTTTGTTTTGTT
TGTTTCATTTTGTTTTAAGACAAGAATCTTACTGTGTAGCCCTGGAACTCACCATGTAGACCAGGCTAACCTCTACCACAAAGACATCTACTTG
CCTCTGCCTCCCAAATGCTGAGATTAAAGGCGTGTGCCACTACACCCAGCTCGTGTTTTCTTTTTAAGTATGTATGTAAATTGTGTGTTTGTGT
GTGCCTGCATAAGTTTATGTATACTTTATGTGTGCAGGTACCCTCAGAGGCCAGAACAGGGTGTCAGAGCCCTTGTAACTGGAGTTTGACGGTC
GTGAGCTGCGCATGTAAGTACTGGGAACTCAATCAGGGCCTTCTGCACGCTTAACTGTTGAGCCACCTCTCCAGCTTGTGTTTAAACATTTTCA
AGTGTTTCTTGTTTTAAACAACCCCACTTCCTTGCACACACCTTTGTGAAGTTGTGCCAAAGGACTTAGACCTCCTACTTTTCCTTTAGTTGGT
GCTGGCCCTGCTCCATGTGGCCCTGGGAGTTCTCCAGAGCCTCCCGTGCATCCCATTAGTCACGTGGTGTTATAGCACAGAGCCTTTCACTGGA
AATGAAGCCTCAAGTTATGGGAATGTCCCGAGATGTTTTTATTAGTGTAAGATAGGCTTTTGATGGAGGCTAGACAGTACATTGGATTGAAAAC
TAACTAGCCTCCATTCTTCCATCAGCCTATGTTCAAGCTCCAAGGTGGGCACAACAGCCCCATCCTTTGATACAGAGTCTGTTCCCTCATGTGC
TAGAAGCCAGGGCTTTTCTACATTTGGGTTTTATTCAGCATGGAACGCAGTTTTTTAAAAAAAGATTTATTTATTTATTTAATGTATGTGAGTA
CACTGTAGCTGTACAGATGGTTGTGAGCCTTCATGTAGTTGTTGGGAATTGAATTTTTAGGACCTCTGCTCACTCTCCTCGGCCCTGCTCACTC
CAGCCCAAAGATTTACTATTATACATAAGTACACTGTAGCTGACTTCAGACGCATCAGAAGAGGGCATCAGATCTCATTACAGGTGGGTGTGAG
CCACCATGTGGTTGCTGGGATTTGAACTCAGGGCCTTCAGAAGAGCACTCAGTGCTCTTACGCGATGAGCCATCTCACCAGCCCAGAATGTGAT
TTTGAGGTGCAGATAGATGGTTCTATGTGTTTATCAGGGTCTCTGGGTGCCTGAAGGCCGCTGAGGCTGCTTATATTAATAGCTTGCTAAGTTC
AGTAACTTTCAGACTTTCAGGTTTGATGTTTTCACTGGTTATTTTTTTAGTGCACTCTTTAGAGTTACAGAATTTTCATTGCACTTTGACAAAG
GCAGAATGACTCTCACCAGTCCTCACAAGAGGGCAGGGGGTGTCGTTCTATGCCCACTCAAGTGTTATGTAAAATGGTATCAGGACTTCCTCAG
CCTCTCAACCGAGTCTAGACAGTCCTAGGACCATCTCAGAGTTCATGAGAAAAAAAATGCACAAAGTAGAAGTGGCCACAGGGTTCCATCTGGA
CCTGATTTTTACCCAGAGGTAGGACCAGCTGTATCAAACCGAGGACCCTGAGCTTAGCCAAAGGGTTTAGATCCCGTGGTCTCCCTACAATGAG
AGGTAGATGCCAAGGAGGTACAGCACGCATGATGCCCTCCTACAGAGGTCTTTTTTGCACACATTTCTGGTCCCTGTAGAAATACAGCCTAGCC
CTGCATATGTGCCAGAGTTGTAGGATCTTTAAGATTCACTCCAGTGCTACAGCTTTCATTTCTTGTTCTGTTTTGTACAGCTTCACCATGGCAG
ACGATGATCCCTACGGAACTGGGCAAGTAAGTCCTCAGCTCACTAGCAACTTTGTTTCAACGCGTTCTTATCCCAAACACACAGAGGCAACGCT
AGTAACTCAGTATCAAGCAAACCCTAAATTAGTAAACACTACTCTTAATTTATTTTCAGTTTTTCTGTTCTTCCAGAGTTGGAGAGGGCCCTTT
CTTATGTATTTTTGCATAAAACCGTATATACTACATGCCAGAAAGTAGCCACTGAGTCAGTTGTGGTGTTGAAGGTCTGACCCCAGTATTTCAT
ATGGTGACTGGTATATTAAAAAAATTGCTATTCAGTGAGATTTCTAGGGCTGCTATTAACATCGCTACAAACCTGGTGACTTGACACAATAGAA
GTGTATCCTTTCATAGGTCTCGAGGCAAGAACTCTGAATTTGGGGCTGGCAGAATAGTTCCCACCTCCTACCACTGCTGGGTGTGTTTACAGAT
CCTTGGCATCACACCAGCCACAGTCTGTTCTTGTGTGGCTTTCCTCCACGTCTTCACACATACCATCTCCTCTCTCTTAAAAACACATCACTTT
TTGGATGTAGGATCCATCTCTGTCTAGTGTGAATTCATCTTAACATATATAAAGGCAAAGGCTGCCGTGTCCAAATAAGTCACATCAAGGAGGA
GCACTTATGAATTCTGAATATATTTTTTTTCAAAGGTACTGTAGTTAAGCCCACTGCAGTCTGTGGGAGTGAGTGTGCTTTGAGAGCGGAGCAG
TGTTTATCATCACGTCATTGTCGCTGTCATCATAGGTGTGACAGCAGCCATGACACAGACTGACACGAGGATATCTGTATCTTCACTTTCCTTC
TAGTTTTTTCTCTTAAAAAATCTAAACCCATAGAAGTGTTAGACAGTCTGATGATACTTCCATTTCTTTCTCTTCTCCCCATAGAGAATATAGG
GAGCATAGAACTGATTCTTGAAAGCAGTTTCTAGCTGCTAGCATGATTACAGTGGCAATATTAAAAGTGGACTCTAATGGTCTATTAAATTATT
TAAAATACTGGTATCTCTTTGCTCCTCAGCTATCTACTGAGACTTTATAGAAGACTTTATGCCCAGAACATTAGCATCCTAGAGACGAAGGGAC
AGGTGATATGCTCACTGGGCTCAGGACTGCATCCCGGCTGGAGTAACGGAACCACACTTTACACAGTCTGTGGATGTTGCCATGGTTCACTGAG
AGACAGAAGCAGTTGGAGGGATTGCTGTGTGTTCCTTTAGGGAAAGAGCAGGTTAAGGATGACTTTGCAAAGGAGGACTAAGCTGAGCTTTGAA
AGCTACAACCAGTGCAGGGGCACTATTTACCAAGGTAGCCCTGCCACAACATTCATTACTACTTCACATTTAAATTTTGGTGGTAAACACCCGA
GACTACCATTTCCCAAATTTATGCTCGCAAAGAGTCAGCCAAAAATAAAACTGAAAATGAAATATTTGGGGGAGACCTGGTGTTTCTATAGTCA
CACAGCCCCAGAGCCGCAGTCAGAGCTGCCATGGCCTGCACCGTAGTGACAGCTGCAGTGGCCTCAGAGCTGCAGTCAGAGCCGCAGTGGCCCT
CTTCTTCTCACCCAGCGGGCGGAGTGAGCATGCACCATCCGCTCCTGACCATGTTATAAAGATGGTGGTACTGGGTAAACCTAAGGATGTTTTC
TTTTATGGCAGCATAAAAGACACCTGGCAACCATACGCTCAAACCCCTTGTTTAACCTTTCACTTTTAATGGGCCTCCTCTAACACAGGCCAGC
CCTCACCACCATGAGAGCTGTCTGCTTGGTTTCAAACCATGCTTTTCTGTCTGTACACAGGAAATCTTAGCAACCAGAGATGGTCTTTTATGCC
ACAAAACAGTAAGACCTGCCAACCACAGTCATTTTCAAAGTGTCTCATGTGGGCAGCAGTTCTGAGCTCATTAGCACCGTCGCCATCTTCTGAT
AGTTCAGTTGTGCGGCAAGCGGAGGCCCTTGGAGGCTTTTAAGTTTTTGTCTTTGGGTTGGTTTTTTCTTCCTGTCTTAAGCTTTAAGTCTATC
TCTTCTAAAGTTTCCGATTTTTCAGGGAGGTGTCCTCCGTGTTTTAGCCACACTGGAGTTTGTTCTCCACCTGTCACCAAGCAAACACCACATG
CCGCCTTCAACAGGACGTCTGTCTTAGTGGGCTTCTCCCCCAGCCAGATCACTCCAGACAGACGCGCAACCACAGCAAAGGACTTCTTTGTGAG
GTCGTTCTCTTTGCTCAGAGATGGAGGCCTGGAGCTTATTATAAAACCTAGTTTCCCATTCAGTGTGTCTTTCATGGTTAATAGCGAATGGCAC
AGATGCGCTTTGCTTTTAAGGATTGAAATATAACACACTCTTTGCTCCTCAGTTGAAAATTTGCATCCCTCAAAAGTGGATTTGAAGTGACTCG
CATTTAAACATCACATTTTAGGGGCCAGTAAGATTTTTCAGAGAGCAAAGTGCTTACTAAGTTAGCTTGACTTTGACTTCCTCCACTACCCACA
CATAAGCACACTCATGTTACACACACACACATATCCATCTCATGTTAAACACACATACTACACATACATATGCACTCTTACACTATGCACACAT
ACACACCTTACACAAACATATATGTCCATACACTCAAACACACACACTCACATACTCCCACTACACATACATACACTTTCACACTATACACACA
TACATGCACACACACTCACACTACATATACATACATACTTTCATACTACACATGCATACACACATACTACGCATACATATACACTCTCATACCA
CACACACATACACTCACACTACACACACACATACACATTCTCACACTACACACACAAATGTAATAAACATGCAAGCATTACTCTTCTTATTCTT
GATTTAACTAGACATAGTAGGATTTGAGGTACCCAAGAATATCTAGTGATTTACTAATAAGGACAAAAAATCCCAAAAACCAAAAACCAAAAAA
AAAAAGAAAGAAAGAAAAAAACCGTCTAGCTTGCAAGCTGACCAGAGAAAGTAAAAGAAATAATTTGGTTTCTATGTTGTCCATAAACATTTCA
ACAAGTCACTTCTGGTCAGTGACACAGATAGTAAGCTCACACATACAAATGTGATTTTCTAAATGAGCTAACTCGGAAAATTCCTTTTGGAGTA
ATTATTAGTCATAACTTACCAAGGTAGAGCTTAATTCTATCAAAACGTTATATCTTCACTTTTTCAGTTTTCTTAGTTGTGTGTTGTTTCTGTA
ACAAGTTACCATAGTATTTGAAACAACACAAATATCTTCTAAAGATTAGAATAAAAATGTCAGAGTCGAGGCATCAACAGGGCTACACTCCTAG
CAGCGTTTATTTCTTCTCTTCCAAGCTCTAGCCGCTGCCTGTAAGTCCTGCTATCATCCCCCACTCCTCCCTCTGTACGCCTGGGTGAGTCCTG
CTAGCATCCCTGACTCCTGCTTCTGTAGGCACACTGCACTTTGATTTCTCACTCTCCTAAGTAGCTCACTTGGATATTCCAGCATAATCCTGTC
ATTTAATTGTCCTACATTGATGGCACATGCAATATATCTTTTGCCACAGAAACCATAGGTTCTGGGGAATAGGAAGGTTCTGTGGAGTAGCATG
TAGACATCTTTAGAGCCATTGTTCACTGTCCCTTCATCTACTACATTCAGGTATTTATATTGTAATAGAAAATAGAGCTCGGCAGTGGTGGCAC
ATGCCTTTAATCTCAGCACTTGGGAGGTAGAGGCAGGTGGATTTCTCAGTTCGAGGCCAGCCTGGTCTACAAAGTGAGTTCCAGGACAGCCAGG
GCTACACAGAGAGACCCTGTCTCGATAAAAAAAAAAGAAAGAAACAAAGAAAGAAAGAAAGAAAGAAAGAAAGAAAGAAAGAAAGAAAGAAGAC
GACGACGACGACGACGACGACGACGAAAAGAAAAACAAAATAGAGGCGGGAGAAAGTGAGCCCTCTGCTCCATCATGTTCTCAGTTGTATTGCC
AGTCATTTTGTAAGTTGTCTAACTCCTGCCTGTAATTCTTTCAAGGCCTTGAAGTTCTGTAGCTCAGTAAGTAATCTATTTATCATCAGAACAG
AAGCATACATATCTAAGATCATGTGCACGTATGTGCTGTCTGTCTGTACGTGTACATTCACAAGTGTGTGGAGGCTGAAGGTGGCTCCCCATCT
TTGATTTTGAGAAACCGTCTCTAACCTAACCTACAGCTCCTGGATTCAGCTAAGACCTCCAGCCAGCAAATCTTCATGTCTCCACCTTCCTGTA
GCTGGGACTGCAGACAACTGCCGCCAGCCCAGCTTTTCACTCGCGTACTGGGGCTCTGGACTCAGGTTCTTGTGCTCTACAGCAAGCACCTTAT
CAACTGGGCCATCTCCCCACCCTCTCGGATGTAATGAACTCAAGTTCCTAAGAGGGTGCGAGATTCAGATCACTTACTCTACACATTTTTCTGA
AGGGTCCAGCATGCCTGAAGCTCTCTTGGCTCCGAAGATGCACAGCTTGCGGGATCCACCAGGCCCTTGTCTTTAGGGACCCCATAGCCATTAT
TCTCCTCAAATTTCTTTGTTACATAAGTTACACACACAGGCGCCTAAGAAGGTCCCAGAAAGGCACGTTAAAATTTTGATTTCAGGAATCCTAT
TTTTGCGTTTTTATTTCTAGTTTTCAATCATGTAATTAGTTTTCTGCCTTTGTGTATTAGTGCAAGAAGAGAAATTAAAATATTTGTATTTTCT
TCTTTACAGATGTTTCATTTGAACACTGCTTTGACTCACTCAATATTTAATCCAGAATTATATTCACCAGAAATACCACTGTCAACAGGTAAGA
AAAACACCCCCTTTTCCTGTTGTTTTGCTTTCCCTTAAAAATATTTGGGGTCAGGAGTCAGGCAGATGTGGATCAACCAAACTAAGTATATATG
AAATTACTATAAGGAAATCTGTTGCTTTGTGGGTGCTTGGGACCTACCCCAGCCCCTCTGGAAGAGCCACAAGCACTTTTAACCACAGAACCAT
CTCTTTAGCCACAACAGTCTCTTTTAAAATTCAACAATATTCATAGGAAGGAAGTGGTGATCCGTCTTTTAAAACCACCCTTTCAGTATGCTTT
CTGGAAATGTGTCTCACAAAAATGCTCCGGCGTGCGCGCACACACATCGTTTTAGCAACTGTGGTTTCTGGTCTCTGTTAGAATACCCTGAAGC
CCTGTCTTCACCCCGTGTAAGGACTTTCCTCTTCTCCGTATCTCATTACGCATTAGTGCTCCTTTTTGCTTTACCGCAGCTCACAGAGAGCCAG
TCTTAGCCCCTCAGTTCTGTATCTGAGGGCCAGTCTTGTCAGCCAGGACACGAGGCTCTGGGGAAACAGTAGGAAAGTAACCTTTATATTCTGA
CCAGACCAAGCGTACAGAACTAATGCTCCTGACATGACTGCTAATTAACGAACCCACAAACTGTTTAAGGTCCCTTTATCCTATAGCGCACGGG
CTTCCTTTAGACTCATTATTGACTGGCATGTCTGTAGCCTTGGCCTGATGGCTCTTTTGCTGCTTTGATCACCTGTTGGAGCTAACCCGTCTTC
AGCAGCCACTTGTTAGTTGGTGGAGATGCGTATGCAACTCATTGTCCATTACTGAGGTGGACAGCAGGTGCTTTGTGTGCTCCGGATTTCTGTG
TCTCGTGATGTAGTTTTTATCATTCTCATTTAAAATGACTATGGTTTTGCCAGCGCCTGCCTTACGGAAAGTTTTCTACAGACGTGGGAGGAAG
GAGAGTTAGTATGTCACCCTCCTCACTGGTGCCCAGAAGTTACATCACGCAGCCCTGTCTGTCCTTACCCCAAATAACCACCACAGTTTATCTG
TAATCTTAAGCTGTCCAACACGCTCTAGGTCCCCTTCTTGTGACCAAGTAAATGCAGGGGAGGCAACCGTGTTTCCTGTGAACTTCTAAACTCT
GAGACCCCAAGAGCTTCCTTTAGCATTTCTGCTTTATTGAAAATGTTGCAGACTCGGTTCTGCCCTCAGGGAAAGCACAGGTTTCTAGAAAATG
CCTCTAATTAGTTAGAATACTAAACAGCATCCTGCAGGCAGATCATATTCTAATGTGTTCTCCTTTTCCTTTGGTTTATGGACTTAGAAATAAA
GTTATCCAAAGAATCATCTCAGAAAGTCGTTGGTGTTCATATCATTGCGCTTCGGGAATCTTCTTTTCCATAGTGTTAGAGAATCCTTCAGAAT
TGCATCCAAGATGCATTCTTTTCCTGTTGTAATGGCTCCTTGCTTGCACTAGCTCTCCGGCTCTGTTTCAGTCCGTTGTCCTGTTTTTATAGTC
CAAGATGGATGGGACATTTTGTTCAGATTTTAAAGGACTAGAAAGGCTATCTGTGACACTGTTTGCTTGTAATTTTCATGTGGATTTTTTTTCA
ATCTTCTCAACTTCTTAAAATCTCAACGTATTAGTATTTCTAACCCTGTGTTGGGAACTCTCTAACAAGGGACAGCTGATAGGTCTGTGTCATT
TTGCTGTGATTTAATTTTCTTTTTACAATGGAAGATCATTTTTTTTCTGGCACAGAAATGTAGCCAACAGAATATTAGCCTTAAAACAAGTATC
TGTGTATCAGCTCCAGTGTTCTACTTCTCTTACCCTTGGGTGTGCTCTTCTAAGGGTAATGATTGCCCCCTCTCCCCATGGAACTTCACATATG
TAAGGAAAACCTAAAACCAATCCTGTGAGTGTTTCAGACGGGCTGTTAAGAAGGAAGAGACTTGGAGCCTAACACGAGGATGTGTCCTCTGCCT
CCATATTTCAGCAGCATCTTATCCAAATGCTACCACCTTCCTGGCCTGGCTTCTCAGTAGGCTCTTTTGGATGCCTAGAGGAAATGTATATTTT
TAATATTGTTATACAAGCTGTCTGATATGCCTAGCATGAAATCTTTTTCTAATCAGTTTTATTTCCTCATAAAAGTTAAATTCGAGCAATTTTC
TTTTTGAAATTTAAAGTTAACAGTTACCTTTCTAAAAGTTTAAAGTGTCAAGAGATGAATCCCATGAGACAGAAGAAGGTAATTTGCTACATTA
CTGACCCCAGTAACCACCCATCTGTCTTTCTTATTTGGCTTCAAATTAGCTTTCCAATTGCTTTCCTGGTACAGTCTTTAGCCTTCCGTGTTAT
CTGATCAGTCATAATATTGAGAAGGTAAAATGTATTCTCTTTGAAGTTACAGTTTATTAAAAATTACAACCTGAACTTTTATCTCACCTCAAAT
TTACATATGCAAAATAACCCTTAACCTTCAGCCGAGAAAGTGAATTTTCAAATGAGCACAGAAGTGTTCTTCTAGACTGTCTTCAGCTTCAAGT
CTTAATTTTCACTCAAACAACTATAAAATGTCGAATATGTTTGTGTATTACACAGCTATTGGTTATCATACTGGGACATAGAAGCCATCTCACG
CCCTTATCGAATTTTTTATCATAACATTTATATTTATTTATTTTGAAATTTCAAGTCAGATATCATTGGTATATAAATTGAGCCTTTAGGTCCT
TTTCATAGTAATTAATTGGCATGCATTTATCTTCAATTACATTATTTTTGGTATTTTATCATAACTCTGGGCTGTTTTAGAACAGAGTTCTTAT
GAGCACACACTGTTAGATACTTTATTATATCTGCATCAAAAGCTGTGAGTAAGATGCCAGGGTAGTGGTCCATGCTTTTAATCCCAGCACTTGG
CAAGAAAATCTCTGTGAGTTTGAGGCCAACTTTGTCTACATAGTGAGCTCCAGGCGAGCCAAGGATACATGGTGAGTCTCTGTCTAACAAAACA
AAACAAGCCATGGAGAAGCAAGAAAAGTTCTTCAGGTCTCACAGAGGGCAAGGAAGAAGCCAGTTAGGCAACAGTGGCTGTCCCTTGGCTTGAA
GTAAGGGAAAGACTACATTTCAGGAGCACTGTTAGGAGAGGCAGGTCGCCCAGGATCTCCCTTCTGTGGGTTTCTAGCTTCAGTTCTTCATTTA
CTCCTGGGCAGTTCTCTTTGAGGGAGGAAAGTAGTCTGCAGGTGAAGGCCACCTGACACCACAGCACTCAGAGCTGCTCCCTGCACTCAGCTGT
ATTGTGCCCTAAATGTTTGTTGACACATGAGTCTGAGGACGGCTCGGATCAGAATCTCTGTCAGCATTCAGACCTTTTGTTCAGTGCCAGTGGT
GTACCATTACCATTCAGAAAAAGCCACTTATTTACCGTTAGTCACTTAAACTATAAAACTANNNNNNNNNNNNNNNNNNNNATCAGACACTCTC
ATCTGGCTTCCTTGGGCACTAAGCAAAGCACTTATGAGCAAAAAATAAATATTTTTTAAAACATTTATTTAATATTTCAAATGATAACACAGAT
CAGTAGAACACACTTCACCTATAAAAATTGATTTGAAATCTCTGTTTAAAGCATAACTTCACCATTTGTCTTGCTTTGTATTTCATACAAGATT
CCAGAAGTCTCACAGTTTTCTTTTGGTTTTCTTGTGTTTTCCTTGTTTTTAACAGATGGCCCATACCTTCAAATATTAGAGCAACCAAAACAGG
TAAGCGTAAAGCCGTCGGCTGTTTCGTAATATTACATCTCATGGTAAGGCTCGGTCTCTGAGTAATAGTTAAATCTGATGACAGAGGTAAGGGT
TTTATCTGAATAATCGGCAGCCTGCCGTGTTATGCAATAACTTCCTGTGTCTACATTTTTCTTACAAATGGCTTTGAAACATCAGTTAATTATA
AATCCTTGCTAGTCTGTCCTTTTTCCAGCTTAGCTCATCAGCTGCTTCCAGGGAAAGCACTAAAGAGTCTCATTCATGAAGAAATACTCACACA
CACACACGTTCACAGCCACTCTGACGCTCTATTGCTCATGGCCATGCCCACAGGGTACCCCTCCTGCCTTCATAAGCTCTGGCCTTTTCTGTGC
ATAGAATGTCTAGCTTAACGCAGTAAACCCACTGCGACACATTAGAGTGGACTGGGAGATCTCCCCTCCACCCCTCTGGCTTTAATCCCAGCCT
TTTTGAAGCAAGGAGAGGATTTTTTTGTTTGGTTGTTGTTGTTGTTTTTGTTTGTGTTTTTGTCTGGCTCTTTCGCTTTCTACTAAATATTGTG
TTCACCAGCTGAAGATTAAAATAGCCTCAAAACTGCAAATATGCTCATTAGAAGTTGCATGCCTCTAGTACTGTAAGAGAGAAGAGAATCTTCA
ATATCTGCCGACAGAACATTTCAGTCATTATGGGATGGAAGTTATGGTATAGCCTCCCACACAGTGAGAACCAAGTCACTAGATCTCTCGGTGG
ATATGCTCATCACAGCCGTGGCGCTGAAGGACTGTATACCTGGCCTACCGAGTTGTGTCACCCAATGTCTTTTCTAGGCTGTTCCTTCTTCTAA
CTCAGGAGAGTGGGTTAACACATTGTGTTGTATGACACTCAATGCTAAGAAGTGAATGCTGACAGCATTCGGTGCTGTGCTGTAGAATTCTGAT
GTGGTCAGCTGTTAAATAGGAGAAACATCTTAGGGATGTCCATGCTACTTTGTAAAGCTGAAATCAGATGGAAATGTTAATTATAAAGACAGGA
ATGTCACATGGAACTTTGGGGCATTGTTTGGTTGCTTATAGGCGGCCTCTTAACACACACACACACACACACACACACACACACACACACACAC
ACGCACACACACATACTTATACACATGTACACCCTCCCTGGGCTGCAGATAACACCTGCCCTTGCTTTATCTCCACGTCCATCTTTGTAAAGAT
GATTTCTCACATAAACTCGCTGTTCTCGGAGCTCTTCAGTAGAGTTGTGTTCTGTGAAATTCTGCCCAGTAATCTGGAATCCTGGAAAATGTCC
TAGCCTGGAAGATTTGGTGAATTCATAAAACTGATGAGTCCAAAATGTACCTACACATCAAATAGATTTTTCAGATCCTTTAGAAAATTTTAAA
TAATTTATTTAATTTTATTTTGTGCGCATTGGTGTGATGGCGTCAGAGCCCTTGCAACTGGAATTACAGACACTTGTGAGCTATGACGTGGGTG
CTGGGAATTGAGCGCAGGTTCCTTGGGAGAGCGGACAGTGCTCTGCTGAAACATTTCTCTAGCTCCCCTTTTTAACAATATTAAAATTGCATTG
ATTTATTTTTTAATGTGTTGCTCTCTTTGCCCCCACCCCGTGTGTGTGTGTGTGTGTGTGTGTGTGTTATTATGAACCCCAAAGTCATAGGTAT
TTATGATGTGTTTGAACAGCTAGTATAGTTCCAGAATTTAAAATTTAATTCATTTTGTGTGAATATATAAAATCATATATGTATAAAACATCAT
GTTTGATTGCCAGTCCGATTTTATTTTGGTATCTGGCTTTAGTGAAAGTAAGAAGGTTGTATGTAAGCCAGGCTTTTTCACTGGATGTATGGAG
TCTCTCTGAGGTTAATGACTCATTTGAAGTAAATGGCGTTCCTGAGATGAACAGCGATAGATGCTGAACGTTTAATTTCAGAATACTAGAATTA
GAAATGTGTCCAGAGCGTGCATTTTAACACTTCCTAATTTTACGTTGTTATTTAATTTAAAACATTAGTTTATTTCTGAGTCAAAAAATGTCTG
TAGTCATTGCTGGCTTCAAACTGGTGCCAGTTTTCCTACCTCCCCTCTCCAAGTGATAAGATGACAGGTGTGAGCCACCATGCCCAGCTCAAAG
GCATCGATGCTCCAGCCGAACAGCGCAATACACATTACAACCTTCACAATAAATCCTCCCTTGGATAGGACGTTCAATATTCTTATTCCACCGA
GAATAATACACAGTGTCACACGCTGTGCAATAAATCATGCTGGCTACAGCGTTAAACGTCACTCCCCATAAGGTATGAGAGGTACAGGTACAGA
TTTTTCCAATGCCACAAAGAAATTTTGTAATTAAAGATAAGGAACCACTATGTTAAGTTTTTGAGTTAGAAATGTTCTTTTTCTTGGAGGCCTT
TTGGGCACAGATGATGAAAATGAACCAAACCACACACTACATGTCTTTAGACTGTCCAGTTCCGTGGCATGTTTCTCACTGTTAACTGTTTATT
CATGTTGTGTTGTGTTGTGTTGTGCTGTGTGAGTTTTGTACTGCTTTGTTTCGAGGCAGGGTCTCAGCAGTGTAGCCTTGGCTGGCCCCAAACT
CACAGAGCTCCATTTACGTGCCTCTGCCTCACAAGCACTGGGGTAAGATGTGTGGCAGTTATAATCTCAAGGTGTGTTTGAATTTTCTCAGTTA
AGTGGATCCCCATCCTAACGGCAGCCCATTTGGGGCTCTTGGTGTTATACATGCTATTATTGTATAGTTTGGGGGTGATTGAGTTCAAGCGTTA
TCCAGACTGGATCCCAGTAATATTAATTTTTACAAACTTTATGATCTTATTCCTTGAAGTGCTCAGAAGACTGCTAGAGTCACAGTGTCGTAGC
ATACAGCATGTCTGTAACACAGATGCACTTAATCCTCCAGAGACTTCACAACCGCCACCAGGAGAAGGGGGATGTGGGGGGCGACCACCCTCTC
AGAGTCGAGGAGGAGGAGGAATGGGGTGAGGAACTTGGGGGGGTGATGTAAATAAATAAAATAATTCTTAAAAAGACACCTTTATGGTCTGTGA
TCAGAGCAGATAAACGAACTCCTTTAAGTAATTAGCATAACTTTTACTGATGGCAGAGCGGAGAGCTTGGCAGCAGCCATTGGTAACGAGAGGC
TCCAGTTGAATAAAGTGCTAACGTATATCCAAAGCCTTAAACTCTTCATCACTCCTGTGTTGAAGGTGGGTGCCTGGCTTCTCCGCCACTGCTC
AGTGCATCATTTTCATACTGGCTGTTAGAAAGCATTTCTCCGTCTGCAGAGTTGTCTTCCACAGTTTTGCATGTTTAGAGACGTGTGTGTACAT
CTGTCATTGTTGCGTTCAGTAAATACCGCTGTGTTCACCTCTGCACAGGGGATTTCGATTCCGCTATGTGTGTGAAGGCCCATCACACGGAGGG
CTTCCGGGAGCCTCTAGTGAGAAGAACAAGAAATCCTACCCACAGGTCAAAGTAAGTTTGAAACCTCTGCTCTCACTGCCCAGGAGCAAAGCCA
AAACGTTCAAGTGAATGTGACTGGGATCTTCCTTTGCCCCTCAGCGGGAGTGTTTACATGGTTCTTAGAATTTTTAAATAGAAATTCACCTCAT
GAAAATGACGAATCGTCAAAACACCATTAACATCTCCATTTAAGTGAGATGGTTCCAACATCAACTAAGAAAAACTTAACTACCTCTCCAGTCA
TCTTAAAGCTGTCTTTTCATCTTAAAGTATTTTTAAATATTCTCTGATGTTTTAGTAAAACTTGCAGCCAGGTGTCATAGTGCATACCTGTAGT
CACTGGACTTAAGAGGCTAGCAACAGGACTGTTAGATATCCCAAGCTACCCTGAGCTACATAGCATGCTTGAGCTCAGCCTGGGCTAGGTGAGA
CCCTTATCTCGTAAGAAAACGAAGAAAATAAAAAATGTCTTTCAGCCAAATTTCAAATACTGGGTTTATAGAGATTTCATGACAAGACCCTTGA
CAGAATATATCGGCTATCGAGTATATTAATTATTTAAGATCATAAGTTCAGACCCAAGTGATGGAATAAACAGGTTGTAAGCAGGCTTATCTCA
CTGACCAAACATATAAATACACAAATACCACTAGCATGTACTTCACAAGGTCTTTTACAGTTTCAATGAGGAAATATGCTTAAGTTCTCTGTAG
AGAGCCTGAGAGAAACCGTGTGCAGCTACCATGAATGAGTTCTGTAGCATGGGGGGCAGAGGGGTGGGGGAAGCTTCACTGTCTTTACCTTGGG
GTGTCGGGTGTAGCTTCGCTGCTCTTCGCTTTTTACCTTCTTGATCTAGTTGAGACTACCTTCCTCTAGTTCAGAGTTTCTACTTTCCTAATCC
AGTGTTGTGTTTCAGAGACTGACAGTGATTTTCCAAGGAAACAGTTAAAAACTTTCGAGTGGTTTAAAAGGTTTTATTTATTTGCTAATCTGGG
CTTAGACTAAATGTTACCAGCAGTAAACGTTATGGCACTCCAGCCTCTGTCTTTCTGCTGGCTTGAGATCTCAGACGCTCTTGCCCTCAGCTGA
GCTGACACTGGGGAAGTTATCTTCAGTTTTGCCTCCTAGGTTGCTTGTCCCCCTTTTAATGTTGTACTTCCAGGCTCAGGCCTTCCAGGTTCTT
GCCCTCTCTCTAACACAAAACAGCCATACCATCCTTTATCTTTCCAGTCAAAAAAATGTGAAAAAGCCATACCATCCTTTATCTTTCCACTGAA
ACTCAGCTCCTGGCTTGGGTCCTGAGAGTCACATTTTCATTTTAAGGCTCCCAGCTCTAGTTGTGGAATGAGCCGTTTTTTTTTTCTCAGTAAA
GGTAACTGAACGTGGAAGTGCAGTGGCTTTAGTGCACAGAAGGCCACAGTTATATTAGACAGTGTTTTTCAGAGAAGTCGAACCAGGTCGGTGA
AGACAGGGAGATAGTTAGATGGTTGACTGAGCGATAGCGGAGGAGATGAGAGTAATCGGCCTCAGCTGTGACGATTATGAACGACACCAAGACC
CACTCTGCAGCGTCTGTAAAATGTGCGCAGGGACACTGCTAGGATTCCAGCACAGAAGCCTGGGCGCCCTGTAACCAAGGGGACTCTTCACTAA
ATCTGGGCGTGGCTATGCTTTTCCTCTAAAGGCAGGAGAGGTTGGACTGCTGATGCCCTGGAGGGGCAAGAAATGAAGAGTGTCCTCCCTCCCA
TTCAGGTGGGGCAGTAGCCGGAGGGAGGGAAGGAGAGGATTCTCTGGTGTTTTGGTCCAGCAGGTCTTCTAGCTGAAGGGAAGCTCTTCCATCA
GTCCAGCAAACTTTCCAGTGTCCTCTGGAAACGTGCACACAGCTAGGAAGCAAATGCTTTATCAGCTTTCAGCATCTGCCCAGTGTAGTCGACT
GCACACCGGAAACTTCCCTCAGGACAGTCTGTGGCTTCCAGTCCTGCAGAGGCCAGGCAAGGAATGCACAGCAGAGCACCGGCGAAGAAAACGG
TTATTCCACACCCCTGGTGGTCCTCGTGTCTCCCATCTGTTCCCTCCTGTAGCCTTATTAACACTGAGGAGCTCAGGCTGAGGTTAACTGGCCC
TCTTCAAAGGTGCTGGGCTTCTTTCTCCTGCAGCTCACTGGCCCTCCCATGTGAGCGTGTGCAGCTGAGGGGCCACGCGCCAGCGGCTGAAGAA
CAAGTCAAGAAGGACTGATAAATCAAGTCAGGGAGGATGAGCATTCTGAACAGCAAAGAAGAAATGGGGGCAGAAGTCATCAGGGGTCAGATCT
AGAATTTCCTACCTGTGTGTGTCCTGTACAAATATTGTGTGCAATTATGTGTCCTTATCTGCACCAGGGTCACTGAACATCCCTTGTCAGCACT
GATACTGATCATAACTACTTAAGAACCGATGTTAGGAGAAAAAAGTAGGGTTTTTTTCTCTATTGGCCTTCTACCAAAAAAAAAAGAAAGAAAA
TGTACATTGTGTTCTATTTATGAACTCTTTCCTTCTGAGGCACTAGAATTCTGTGGTCTGCCATGCATGAAAGATGGGAGTGTCTGTAGAAAGA
GGCTTCCAGGGGTATCATGGCTTACTGCATACCCACCCATAATTCATCGTAGAGGTGGGTCCATGTACTTCCTGAGGGAGCGACTCTTCCGTAG
CCTTGCACATGCAAGGCTGTGACTTGCATGAAGTATATGTCTTCACACTCACCTTTTCTTCACCCCCACTGACAATCTCTGTCTCCCTGGACTC
AGGCCCTCTTTCCTGCCCTGCCTCTTCAAGCACCCCTCCCTGCTGTCCTTAGGGAGCGCCCCTGCAAGGTTAGTGGTTGATGTGATGTGAAGAT
TGCAGTGAAGATCTGAAGGCTCTTGAAAGCTTTGGGAACTGGTCATGACTTAAGGCTGCAGAAACCAAGCTTTGCTTTATGGAGCTTGTAAACT
TACATCTTGTTTGGTCGTCTACTTGTTGACTGTTTGAAGCATCAGGTTGACTTGCAAGCTAACTCTAGGAATCTCCCAAAACCAGCACTTCCTA
TTAGAAGTATTTCCACGCTGTCGTAGATGCTGGGATGCAACCTCAGTCGTCACCCCCTCCCCCCACTGAGGTACCACCTTTTCTAGAACTCAGG
CGGCTGTACCATGTGTATGTTCCTCTCGTGTAGTTGAGAAGAAAGAACCTCTTTGGCTCTCTAGGATCTTGTTGTACAACTCTTCACGTTCTTA
TTTCCTCTCTCTCTTTTGATTAATTTCTCTGCGATTACTGGTTCCTATGTCCTCACAGCTATCATCAATGGTTTCTTCAGATTGAAGCATTCCT
TCCACTGCCTTCAGGAGCTCGGGGTGGGTGAATAGTCTCTTTTAAACCAGCTACTTCATATTTCAAAATATTTTAAGCATACAAGTTGTAAAGT
TCTATATAACAAATACCATGTACCAAAATTAATAAATTTAATAGATGGTAATATTTTGCCTATTTGCTATCTGTCTATCTGTTCTCTCTCTCTC
TCTCTCTCTCTCTCTGATTCTCTCTGAAGTACAACATAGTTACAGTGGGAGCCCCTACTCAGCCACTGCTCTGTCCTCTTATCCTCTTTCTTTG
CCCAAAGGAAAACCACTGTTCTGAACATTCTTTATCTGAGATGCATTCATATCATATACTAAAATCACTTTCAAGAAAGTTACTTTTTACTATC
TTTAATCCAAAGTTTTTTATTATATATGGTGGCTCTTTTTCAAACTATATCCAGGTTAAAACTGGGCCCCTGAAGGCTTAAACATTGATTGCTT
TAATCCAGTCTCTTCCTCTTTATTGTTTGACTAAAAATGTCTTCAGTCACAGTGTGGAATTTATCCTGACCCTTAAAGCAAAAAATCATATTCA
TGAGGAAGCTAGGGAAAATAAATATCCAGAATTTAAGAAAGTTTTCCACTTTTAAAAAATGGAGTAATCCACTGTCTAGCCTGTAAGCTCTCCC
TCCAGTGTTTGCTTGGTTCTGACTTTATCTGCGGCTCCTGGGAGAACAGGAGAGACAGCAGAGCTCCTCTGGGCCAAGCCTGTCGCTACCTGTT
CTTGACTCAGCAAGGAGTGCCAGGGAAGGCTCACTGCCCTCCTTTTCCCGAAAGTGGTGCAGAAAAATTTTAAATCAAAGTGTCTTCCTTGAGC
AAATGGAACAGGGCCAAGAGGAGAAGCCACACGTGGCTTTAGGAATGCGCCTGCAGCCCGTCCTCACTTGCTTGAGTCCTTCTGTGCTGGGCAC
CTTGTGAACATTGCACTTGGACATCAGTGACTTTCATTTCTAAAACACAGTTTGTTTCTAATCATCCAATGCCAGGAGGTTGGAACACAGTGAT
GCTAATATGACGATTCTCATCAGCTTTCCTGGCAGCTGCCCTGCCTGCCACTCTCCTGGGGTTTGATGGTTAAAGAACGGGTTTATTGGAATGT
TTCTGTGAACAACACCTTTGGCTCCCTTCTCCCACCTCCTGTAGTGTGCACTCTTACGTGCCTCTCACTGAACAGTCCTTAATGTTACGTTTCC
GTCAAGGTTAATAAAGCCCGGCTGATCTGCTGTAGGCTTGAGGATATCACATATCTTTGCAAAAGCGCCCTTTCCCTTGCACAGGCTTGTCTGC
ATCCGGAGACTTGCCATCCTAAAGATCTAGTTCCAATACTAATTTTCAATAATTTAGCCTCATCCAGTTACCTAACATGCCAGGGGGGAAAGAT
CACCACAACAGATTGGGCATTCAGAACCTACCTAGTTCATAGCATTTATTAGAATGTCCTTTGACACTTCCAAGTGTTTACTGTGCTCAAATAA
AAGAAAAGAGCTATGTTCCCTACCAATACTATAGTCTGTGCTTTGTAACACACACACACACACACACACACACAAACACAGAGTTTATTAAAAC
AAGGGCTTTGTAACCCAAGGTTCATGGATCCTTTTGTGGTTTCAGAAAGGTTTGCAACCTCTGACTCTACACATAGAGTTTTATCCATATTTGT
ATGCATTTTTAAAGACTCGAACATGTGTAAAAGTAATGCTGAAGGCCATTGGCTTTTTTACCACAGCTGATTAAATCGATCTCTACAATTTTGG
AGGGATCCGCCCGGGGGCGGGGGGAGGGCGACTGGCTCTCACTGTGTAGCTTAGGCTGGCCTTGAACTCATCTTCCTACCTCTGCCTCCAGAGT
ATTGAGAAATTCCAGGGATGCAACTGTCTTCAATGTTTTTCTTCTTTCTTTTTTCATTTCAAAAATTTACATTTACACTTTTTAGTTTTACCCA
TTTTAGGCAAGTTGAAGCGCTTTGTGTTTCTCAGCATGTAATTCAAGCTAGTTGCTCTTTAGTATTCCATTGACTGGAAAAGATGTGAGATTTA
GCGCCTCCTCACACACACACACACACCACACCCCACCCCCGCCTTTCTGTTCTCTCCTTCCTCCCCTCTCCTCCCTCTCTCCCACTCCCACCGA
GTCTCTTGGCACTGACTAAATACGATGGTTCAAAACCAAAAACAAAACAGAATCATTTCCCTGATATTCACTCAGCACGGTGGCAAGTCCTGCA
CTACAAGGAGCTAACATCCTCTGCGTGGCCATAATAATCTACACTTCTGCATAACTTTTCTTCTACGTATCCCAGGCGTTTTGCGATTGCCTCC
ATGAGTAAAATACTGACTTGTTAAAAAACAAACAAACAAACAAAAAAACCAGCCCTTTATTTCACTGTTAGCATTAGAAGTTAGAGAAAGCTGC
TTACTGAAGGAAACTGTTGAGCAGTGAGAATTGATCCTGAGAGTTAGGAGAGCCAAGCTCCTGAGGGTTGCAGGTTGTCTCACTTTCCAGGCTT
GGTATGCTGGCCTGCCTCGCCTCCCATTTCCTCTTCCCCTCTCCTGTTTCTCTGCATACTCATTGCTCTTGGGATGCTTGTTCCTAGTGGCAGT
GTTCAGTCTGAAATCATCTCTGATCAAAGTGGTCTCTGTTTGAAGACGATCATTACATTCAGGCCGGGAATCCCGGTTTCGTAAAGCCACATGT
GAAGATTGGTTTTGATCACCCAGAGTCAGTAACCAGAGCCTATGATCCTCATGTCCCCAGCCACCTTCTCCATAGTCAGGGTCTAGGGTCTTTG
GTGGGTCTACGGATGTTCTGCTGGCTCTCCTGCAGTCCCCATCTTCCCCCTCCCACTCTAAGTGGATTTTAGGTCAGTCTTCCAAACAACTTTC
ACTTTCTTGAGGTCTAATGGAGGTGAGTGGGAGGATATCTGGGCAGACATGTGAAACAATCCAGTCTGAAGACCAGGGGAATGCAGAGTAAGTC
CCCAGCATCTTCAGATAACCGAGTTATCTATCCATTGCTTGATAGATAAGTACAAAACAGGTTTGCTTGTATTAGAGAAGACTTGGAATTGAAA
ACAAGCCAGAAGAAGGTAAGGCCAAACACACATTTTTCAAATCACATATCATTGAAATGGTCTAGACATTGGAAAAACAATGATCAGGTGGGAG
AGTGGATAATGACGTTTCTCGCCGTCGAACACAGAACCCTGCAGGCTGGCAGAAAGCGAGAGCTGAAAGCAGAGCAGAGGGTGTCTAGCTGAAG
TCCTGCGACAAACTAACATGGCTGCATGTGTGTGTGTCTCCTTCTCTACGCACAGCTCTCAGATTGGCATGCAGTACTCAGCTTGTGGGCAACA
TATTCACGGTGTTAAGCACCAGCTGTGAACTGATGGCCAGCGTGGGGTGTGACATTACTTCCTAGATTCTCATTGGCTAAGCTTTGTGAACTGA
TGGCCAGCGTGGGGTGTGACGTCACTTCCTAGATTCTCATTGGCTAAGCTTTGTGAACTGATGGCCAGTGTGCAGAGTCTCACTGGCTAAGCTG
CAGCTTCTTCATCTGTAAAGCTGACGTAGAGAACCAACTCAATGGAACATGTGCGTACATTCAGTTGCTCAATGCCGGCACACAGTCAGCCTGG
AAACACAATTACACCCGTTTTCATATAAAATTTCAGGAAATTGGGTACTACTCCAAAAGCTTGTGTTTAGAGCTAACACATGATGTCATACGAA
ACTCACAGAAAACAAGAGGATAGAAACAAGTGTTAGGTTTTGTTAGGTCCCTTCACTGTGTCCTGGTAGGCCCTATGTGTCTAATTTGCACAAC
GCTGTATTCATTCCTAGGTTGCCATAACAAATAACTGCAACCTGGGTCATATTAAACAACAGAGTTTATTGTTCCAAAGTTCTGGAGGCCCAGA
GTCCACAGTGAAGTTGCCAGTGTGGTCTTGCTTCCCACAGAGGCTTCAGGAAAGAGTCAATCCTGGCCTCTTCCAGTTTCGGGTACTTGTTGGC
AGTCCTCAGCAAGCTCTAGCTGCAGCCACTTCTCTGATCTCTGCTTCTGTCTTCACAGCCCTCTTCTCCCTGACTTTCTATCTGAATCTCTCCC
TCCTTTCCCTCGTAAAGACACCAGTCAGCGTGGTTAGGGACCACCACGCTGTGTAGCAGGACCTCTTCTTGACCAATCGCATGTGTAGGAAGCC
TGTGTGTAGATGAAATCATGTTCTTAGCTCCTGCATAGGCCCGAGTCCTGGGAGATGCTCTTCTCACTTACTACAGGCACGATGTGCAGTGGTT
ACAGAGTGTTCCCGAGTGTGTGCATTTAAGGCCATGTCGTTCCGTGCTGTGCTCAGAAAGAAGCCTGTGTGCACCACGTGCTCCCCTGTGTACC
TTTGTTAGATTCAGGTAGTGGAAGAAAATCTCTCAGGTCTGAGCAAATTCCTCTACTACGGTTTTCAACATTTATTCTAGTAGATGGAAGGTGT
GCTAGAAAGTGTCTGGCATCAGACTCTTGGATGGCACTCTGAATTGTAGCATCTGCCAGTTTCCTTCCCATAGTAAATTTCAGGCTGCTAACAT
GAAGTCAACTGGCTTGCAATAAATAAATAAATAGATAAATAAATAAATATTTTAAAAAACAGTGAAAATTTAACTATTCTCAAATTAACTCCAT
GAACTAGTCAACCGGTTTCTAACCTACTAGGTGTTAAGCCAGCAAAGTTAGAGCGAGAGAAGGTGATACATTGTTTCCTGTAGGAGCTACTTTC
TATGCACATGTTACATAATATAAAGATGTGCAGGCTATGAGGGAACCCGATAGGTGTTACAGCTGGGACTTCATTGTGTGATAGTTAAAGAGAG
CTGCGCTTGAGTGGGATTACTGCTGTTTAGAAGCAAGGCTAGACCTCGGTCCCATTCTGCCACTCGCTCGCAGGTGGGGTGTTCTTCAGAAAGT
TGCTAAAGATCTTCAAACTTGGTTTTGTTTATGAGATGAAAGTAGAAACAACTCCTTCACAGGAACCATGCGTGCTTACGTAAAAAACTGACCT
CTTAGAAAGGCAGCTGCCATACTCCTGGCACATCTGCGTCATTAAGTGCTCATTCGGACACGTGTGTAGAAAGAGCCATGGAGATGACTGTTTT
TTTCCTGAGAGCAGAAGCTTGCAGGTACTTTGTCAACAGCACCTAGAAGGATGCCCATCTATAGCACACAGGCAGCACTTACTTGTACAGTGAG
CAGGTCGTCAGTGCTCAGAAGCTGGAAGGATCATCTAAGGAGGCACTTGTCCTCCCATGCTGCCGCTAACCAAGTCACTCGAGTGGCCACCAGG
TCTCCACCTGTCACTGCTGCTCACTCCCCACCAGCCTGCTCCTCTTTGCTTCTCCAGCCTGCCATCACTCCCAGCCAGCACAGCCACATCCCAG
ACCTGGCTGTACACTGTACTGTAGCTCTTAGACATAGCACCAATCTATTCTGGGTTGAAACTGGCTTTTCTGCCTTAGTTTCTAAACCTATTTT
AACTGTCCAATTGCAAAATAACACTTAAAGAAACTAAAAAGGTTTAAGCAAAATAGACATTTATTTATTTTAAAAACTGAAAATTCTAAGGGAA
GAAGTAGAAAAAAGAAACCCAAACAAACAAACAAATTTGCCAAATCCGTAGAAAAACTCCCATTGGGCTTTTTAGCATCTCGACTTCTACCTTA
GAGGCCACTCAGCAGCAGCACGCTCACTGTCAGAAGACCTGCATTCCCAAAGCTGACATCAAAGCTTATGAGACACCCAACTGGCAGTTCCTAG
GCCTTTGGTACTCTGAGTTGTTCCACTGCCTGCCCCTGCAGCAGTGAGACAGGGGATAGGGTTGAGCCCTGTGTCCGTCAGTCGCATACTCTGC
CGCTGGTTCTACAACTGGGATTAGTCACTGACTAGGAACATGGGGAATGAACTGAGGTTAGGATTCACTGAGCAAAGAGCTATGCGCTAGTGAC
ACGCGGATGCTCCAAGATCACAAGGATTAGCTCATTCGTCCATCCCGTGACCAGCCTACAGTTTCTGAACTCTCATTCCTAACAGTTGGTAAAC
CCGGGTTCCTCTCTTGAGAATGACTGTTCCATGCTACTCCTAATATATGAAACTTTTGTTTTTAAAATAAACCAAAAAGTTTTTTGTTTATTTA
CAGATATCACTTGCATTTAATAAAATTTCAAGAAACACAAAGTTATAGGTAGTAAGTCCCCTTCTGTCCAAGATTCCTTTGTTCCCTGGAGAGG
AGAGCTGTTACCGGCTTCTCAAGTGCACTCCCAACCGTGCCTGCCGTGTGTTTACCATGTGTGTTTATACCTGCTGGCACACTTCCACTCACGC
TTCCTCGTGTAAGTAAGCATTAGTGTAGCGCTTGTGAGCACGCATGTAGTCTACTAATCTATGAACCATCTGCTCCTTACTTTTTTCACTTACT
GAGTCTGAAATTTGCTCAGATTAACAGCAAACTCCACAGCTGGCAAATCCAGATTCAAGTCCAGCTCTCTCCCCTCAGTAACTGTGTAACCTTG
AGCTAACTGACCACCTTCTCTGTGCCCCTATCCAACTAGCATAAGAACAACTCCATGTCTCATAGTGTTGTTGGGAGTACTAATTGAATTAGTG
TTGAAAGACCAATGTCTGCTCCAGCGAAGAGTTCTGATGTTGAATTTCCTACCCATCTACTCTGTAAA~GCTGTGTGCTTTCCATGGCTGCTTG
GGGCATGCTGTCTTCTCTAAGCTCCATCTGTCTTCTATCACAGGCCAACCTTAGTAACCCTGCATGATAGTAACTTCTCCCTTATCCTGGGGTC
TTTGTCACTTTGGGATGTTTTGTTATTGAGATAGCTTTTTAAAAATACTTATTCATTTTTATTTTATGTGTCTGAGTGTTTTTGCATACATGTA
TGTATGTGCACCATATGTATCCAGTGCCCACGGGGGCCAGAAGAGGGCGTTCGAATCCCTGAAACTGCCGTTGCAGTTGGTTTTGAGCAGCCAT
GGGGATTTGCTAGAAGAGCAGAAAGTGTTTTTAACCACAGAGCCATTTCTCCAGCCCAGAGATACCATTTTAAAATTATAATCTGTGACTGTTT
TGTTTTGTTTTGTTTTGTTGAAACTAGGTTTTACCATGAAGCCCCCTAGCTGGCCAGCAACCCACTGTATACACAAGTCTGGAATGGGACTCAC
AGAGGTTGGCCTGCCTCTGCCTCCCAAGTGCTGAGATTAAATGCATGTGCCACTGTGTCTAACCCTTTCTTAATGTAGTGTGTTTATTCTTTGA
AGAGTCATAAACTAGATTTTCACCATATTCACTTCCCACTCTTCTTTTTAGCTCTTCTGAGGTCTACCTCCTACCTCCTCTCACTTTCTTCCTT
CCTTCCTCCCTCCCTTCCTTCCTTCCTCCCTTCCTTCCTTCCTTCCTTAATTCATTCATTCCTTTCTTTCTGCCTATGTACTAATGGATATGAC
ACTATCCACTGACCATGGTCATTCCTATAAACATTTTCCTTTCATAAATGTTACTCTATGCATGTCGAGGTCAGACGCAGCTTTCTAAGGTCAG
TTCTCACTTTCTGCCTGTCTCTCTTGTTCCCCACTGTGATGCATCCTCCAGACTTGATGGCCCTGGAGCTCCCAGCTGGTTCTCCTGTCTCTGC
CTCCCATCTCTCTATGGGGTGTTGGGATTATAGATGTGCATCACCTAATAGGGCTTTTTACCTGGGTCCCAGGGTCTCAAACTCAAGTTGCTGT
GATAGGCATATATAACAATGCTTTTACTCATGAGCCTCCCTGGATGCTGAATTTTTTTTTATCATAGCTTTATTTTGACTCTTTATTGTGCCAT
TTGCTTCTTATATGTTTAATGATGTGTTTTTCAATGTGATGACCCTTTCAGAGAGTCTGCCCTGAGTTGTTCCCAGGCCTCGCCCACTTGAGTG
TGTTCTGGGGATGCCTTCCTAATGAGCCCCATGCCGACTCTGATGGCTGCCGTACTATGTCTTACTCAGATGTGCCCCTCCTCATTTCTAGACC
TTATTTCATGTGCATAAGAACTCTGTCTTTTTAGATTCCCTAACACTGTGCTCAGTTAAATTCCTTTTCATTCTTGCGTACTAGGTGATTACCC
CAGTTAGCAATAGAAATATGCAATTAGTATTAGACATAAGAGGAAACATGTCTCTCCCGTTCATCTCTTACTGAGCTCTTGCTGTGTCTCACAG
CCAATCATATTCAGCTCCACCAGGGAGGCGTTGGTATCCTAGTTTTCAATTGGGTTTGATGAACACTTGGCCAAAAACAGCTTGTCTGTCTACC
TTTCTTATGTACATGCACACACACATATCTCCTAATTACTTTCCCCAGCCTACACTTCTCCAGAAAACCAGGCTTCAGATCTCGCTACTTGGTC
GACTTTACCATTTGGATACCTGGTAGGCTTGCTGAGCTCAAAGTTGAATGCTTGATTCCCTACTTGAAGTTGTCCACTCTTACAAAATGGTAAC
CCATCCCCCAGTTTCTCAGACCAAGCGGTCTTCAAGTTATTCTTGACGCTCCTCTTCTTGATCACCACTTATAATCTACCCTACTCGCTATTTC
TTCAAACTAATTAGAATCTACTTGCTTGTTGCCACCTCCACCCCATGAGCCCTGG1ATAAACCATCCGCCTCCTAATGGCTGCCATTTGCATGG
GTGATCCCTGTCTCAAGCAGGTATTGCTAACTGATTACAAATTTAGGGTTTGCTCTATCATTTTTTTTGTTAGATAGCATTTATCTTTTTGTAC
TTTTCAAAGTAGTTTTCTCTTCTTCACACTCACTAAAAAATATTTCAGACTAGTTTAAATGTTTCTAATCTAAAAACACAAAGTCAAAAAATGC
TCTGAAAGAACCAGGGGAAAATGAATGGGGGTGGGTGGGGCGGTAAAGTACTTGTCATACAAGCATGAAGAGCTTGAGTCCCCAGAACCCACAC
GACAGCTCAATGCAGTATAGTAGTGCAAGGCTCTATAATCTCATTGCTCCTGTACAGAAATGGGAGGTAGGCACTGGAGACTCCCTGGAAATTT
ACCTGTGGTCCTGAGCGTGTCAGCTCAGTGCAGTTCTGGATCCTCTTCTCATCCGGGAGCCACGCATCACTCCTGCCATGATTTACTGTGATAT
TTGAACGGTGCTTGTCACCAACCAGCAGAAGTGTTGCATGCCAGCTCTCGTGTCCGCTAGACCTCCCTCCCCCACTGCTAACTGACTGGGCTCA
TCCTCCATCTCCTGCTCTGTCCTGAAATGGCTCATCTGTCCAAGGGACCTGATTCCTTAGCACAGTCGCTTTTCCTAGGTTTTCCTGAGGCTCC
ATCATTCATCTTTTTTGTTGTTGTTGTTTTTAAGTCTTGAGTTCAGACCCTATTTCACACATCTCATTGTTGCCATCATCACACACTCTCCTTC
CCTCCCCATCCTAGGTTTGTATCTCCTTTTCCCAATAAAATGCAAACCCTCATTGCCAATAACACTAGAAGTGCCTACTCAGTTGATTGGTCCT
ACAATACTTCCAAACACATGGGGAGATAGTATGGTTTCAGGATGACGACGCCAGTATCAGCCCTACCAACTTAACAAAGAGTTGCGTTTTCTTT
TTATTTCTTTAACCAAAATTCAGCAGGGAATAATCAACCAGAATAGCGTTTTCATGGGTTACATTCTTTTCCTTCTAGGAAACTGCGTTACCAT
TGAACTATAGGGTTAGAATACTTTGTTTATGATGAATCTTAAGAGTTTTCCTATTCCTGCTGATTTTGTTTTATTTTTGAATGGTGGCATGTAT
CGGCCTCAATTACCTTCCATTTGTGTGAGATAAACTGGACGATTTGCCAACTATCATAAAGTCCGTTTGCTTTATTACCTTTTAATCAGAGAGA
GGAACAAAGAGTTCTGGGATGGTTATTCTGGAATGTGAACCCAGTAAGCTCACGTTGTCACATGGGGTGTTCCATTTCTATATTTGGAGCCCTC
TTAGTCTTCCTCACAGTCACAGAGTACACACATATATGCTTCTCGCACATAGGCTTCGTCCTGGCTCTGCCGTCACAGACACGAATGAGTTTAT
TCCTCTGCCCCGAGGTTACAGAGAGTAGCCAAGCAGCAGACACGTATGAGATGATACTGCGGTGTTTCCTTGGAATTTGGGGAGGATGCTCTGC
AGTGGGGTGCATATGTGTATGTGTGTGTATGTTCCTGAAGTGGGTGGCCTTCCCTGAATGCATTCCTACTGTGTAGTCATTCCAAACTTCTGAT
GGTGGAATGCCAGCATCACAGAGCACTTTTCATACTGGATGTCAGAGCCATGGCCCCTTCACAACACACGTGCTTCACTCTCAGAGCGGTGGGC
CTGTCACACTGCGTGTGCCTCACAGATAACATAATCTTCCAAGCTGGTGATGTCATCCTGGAGAGACAGAGCCAACGATGAGCCCCAGGGGATT
GTTCCTAACCATTTTAACAAGTCTCAGCCCTAAAGTCTTTATGGTTCTTATCAAGTTTTTTGACAAGAAGTGGTAGATAACAAATACTTTAAAT
TCAAATTAATCTTGAAAACTGGAGCGAAGTGCCAAACTCTTCAGCCTTCATGTTGAACAGAGACACACACATCCAGAAAAAAAACAATGAATCC
ACAAAAGTACAGAGCAGAGGGGTGAGTCAGCAGCAGTACATGCGGAGAGAGTAGAGTTGACGTGCCTAGCAAGAGCTAGCAAGGCCTGCAGCTG
CCAAAGAAGTCGCTCCTCACTAGGCAACAGTAATGGAAATATGATGTATAACAGCCAAGAGGTGATAGTCTCATGGCCACCCTGCAGGAAGATC
ACACTAGATGCGTGCAGCCTGCTGGGCAGAGCACAGTAGTCGGCTGTGGGCGGGGCGGGGCCAGATCAGGATGGTTGTGTGACCACACTGGAAT
GGCTTACAGGATGGTCCTGGCTGTCCTCGGTGTTTGAATAGCTATTATAAGAAAATGGAAATGGACCCCACCATCACAAGACAGGGGAAAGAGG
GAGCGGTGAGCTGAAGTCCCACAAGACACCAGCTTCCTGCGAATCACAGCCACCACAGGACGGGGTGACTTTGTCGTCACTGCTCTGAACTAAC
CTGAAAGACGGTAGAGGAAGAAAGGGAGGCTGCGTGGGCTTCAGCTTCACAGCCTCGGAGTCTCTGGCTCTCCATCCCTGTTTTATATGGCATT
AGACAGAGTTTTGCTTAGTAAATCATACCTTTGTAACAAGGGCTGATCAAGATTTGGGTACAAGGAAGTTATAATTTCATAATTTCATGATAGA
TAAGAAAATATAACAATAGATGCTAGTGACTTGCTGGCCATTTTCTCCATGGGTCAAAGTGCATGACTTATTTTTTTACACTAGTATGTCAGTT
TATTAACACAGGCCGAACAGTTTAGTCACAGCTTGAAAGGGTTTGGTTTGGTTTCGTCCTGAAAGACCACGTACAGAGTGATACCAGATTTTTC
CAACAACTTGCTGGCATTATCCTAGAAATCAGTAGAAGTTTTGGTGTTTACCCCTCCATGCCTTCGTCTACATCATGCTATGCAGATGGCACAC
TGCTTTGGTTAACCTCACTCTGACACGCAGATCGTGACTTCTCTTCTGCCATGCTCCTGTCTGGGGGCGTGATGTGACCGATGAAGTGTCACCA
AGAAATAAGACAAAATTTCCCAGGTCTACTCACTGGCAATTCTCTTTATTTAGTAGGGGTTTTCCACACGAAATTGCAGGAGAAGAGTTGAAGT
GCTCTTTGTCTTGTGTACTTTCCTCTGACTTTTCTCCTTCCCAGAGTGGCCCACATTGCCTTGTAAAGGAGCTGTGTTGACCATGATACTGATC
AAAGTCAGAGTGCGTTCTGCCAGTCAGCTAAAGCCTGATTGCTCCTCTGCAGTGGACTTCTGTAGTTAAACTCTGTTTTCCAAACATGAAAACA
CATAAACAAGGACTGGAGATATGGCTCAGCAGCTAAGATCACATACTTCTTCTGCAGAAGACCCAGGTTCCGTTCTAAGCACCCACATCGGGCA
ACTCACAACTGCCTATAACTCCAGCTCCAGGGGACCCAACACACTCCTCTGGCCTCTATGTGCACCTGCACTCATGGGCACATATTCACACATA
TATACATAATTTAAAATAAAAATCTTTTAAAAATTAACGAACATATACCCAGAGAGTAAGTATTTGTCTCTAAGAAGAAATAAATCGGTGATAT
TAATCTGAAACAAATTTCGACATCACAGAATCAAAAATATTTCTGATTATCCTAAATTAAGATCAAAATCATTTTAAGGGAATTCTGGAGAGGT
GGCTCATTGGCTAAGCTCTTCTCTGCTCTTGCAAAAGACCCAGGTTTAGCACCCAGGACCCACATGGCAGCTTGGCAACTCACAGCCATCTGTT
GTCCCAGTGCCAGGAGACCTGACACCCTCTTCTGGCCTCTGCAAGCACCTGCACACACATGGTGCACGTATTTCACGGAAAACAATTATAGACA
TAAAATTCAAATTAATTTTAAAATGTCATTGTAAGATCATGCTAGATAAATTTTGTATAAATCATGTCTGTTTTCAGACAGGGATTCTGTACAA
ATCTAACATGCATTTTAGACGACTTAGTTTTGAAGTTTACACAAACTTTTTTTAAAACTTTGCTTAAAATAGCTAATTATGACTTCATTTGCCT
GTTTTGGCCCTGCCCCCCATGCCCAAAGCCTGCTGACATCCCCAGGACCAATAGTCCCTTATTCTCAGTACTATAATAAGTACTGAGCTCCCAG
GGCAGAGCCTGCACTGCAGTCACTGAAAAGCACTGTGTTGAGTACCCAGACCCAGCCTGGATGGGAAAAGTTAATATCATTGCCTAACATGAAG
GTTTTCTTTGTCTCTAAATTTTGATTTTTCCATGCTATCAGAGGCCCCCTTGCAACAGGTAATCTGAGTGTTGTCTGTTTTCTCCGTCCGTCAT
CGTGGTTAAGTGTATTTTAGATGCTCACAATTGTAAAGCTAAATTGACTTGACAAGGTTCTTTCCGACCCAAATTCTGTGATGTCCGACTCTTT
TTTTCTTCTTTTATTTTGGATTTGTGATTGTTGACCATTGTCCTGTATGTCTTACATATATACTTTAGGGAACCATAAGCATTTTAGAGCTGGA
AGCCATGCTAGCTGTGGGCTAGGACTAGACATTTTGCAAAGAAAGCATTAGGAGACCAAAGTGGTTAGTGATTCAAACAAATTCATAGCAGTAG
CTAAAGACTAAAATTCTGACCATCTTCAGAGGACTAAAAATAATCAAAGTGTTATCTGAAAATTTCTAACTCCAAGCCAGGTATATATATATAT
ATATATATATATATATATATATATATATGTATGTATATATATTTACCCCAAACAAATTAAAGCTTCCTATAATTCTGTACTCATATATGTATAT
GGGGTGTGTGTGTGGAGAGAGAGAGAGACAGGGAAAGAGAAGGGGAATTAAATATTTTTGATAAACGTAAGCTTTGATAAGATCTGCCTGACAC
AAAATACAAAATGCATTTGGTATAAACAGCATCATTCTCCCTTTATACCAAAAACGGAAATTTCTTCGCATTCTATACATTTGAGAATGACACG
TCGGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNCCGTGTTTGCTGCTCTGTACTGGCTATAACCTGCACAAG
TAATGGCCTCAATACAACTGTTTTCTAATAAAAGCATCCGTGATGTTATACCTCTAAACTAAGATTGGTTTATGTAGGTCTGCCATTGAGAGTG
GTTATGGGAGGATTCCTGTCCAGCATTGGGACTGTATCTAAAAACAGCAGTCTAGCTAGAAAAAAACAGCACCTGTACAAAAAAACAAAAACAA
AAACAAAAAAAGCAGCAGACTCAAAATAGGAATCTCACAGGTCTAATGGGAATCCTGAGTACTTCTGCTGCAGAAAAGTATCCTTAAGAGAGCC
CAGGTGAAATAGTGTTCGAAAGAAAAACAGGGACATGTTGTGCAAAAAAGATAGACTATTGCTTTGTTTGTAAGGAGATGGGTCACCCGTACGC
GTTTTAATAGAGTGGTAATGAGCATCACTAATCCCTGAGCTTCAGAGCACAGACTCGGCGTCTGTCTGCAAAGGGGGCTGAGTTGGAGCACTGT
TGTGATTGATTCCTCTTCAGTTAGACTTAGAGTTCAAGCTAAGATGGATAGGCAAGGCAGTGAGATCAGAGGAAAGGGCTGAGCACGGGGGGAG
GGGCAGGGGACGAGGAGGGAAGTGCCCTGGCTCTGAAATTTGGCAGTTAAGTGGAGATAAGAGGCAGCGCACTCCCACACACTAGGACGGTTTT
GGTACAGTGGCTCTGTTCAACAGGAGGCAACGTTAGGTTTAAGAAAAAAGGTAAGCTGGGCGTGGTGGTGCACGCCTTTAATCCTAGCACTCGG
GAAGCAGAGCCAGGCGGATTTCTGACTTCAAGACCACCCTCGTCTTCAGAGTGACTTCCAGGACAGCCAGGGCTACACAGAGAAACCCTGTCTC
GAAAAACCAAAAAAAAACAAAACAAAAAAAAAGAAGGAAGGTAATTAGTTAGGCTTGGGCACATGGAGTGATAAACTGCTGTGAACAGAAGGGC
TTCCTGCTAACGGCTAGAAATGTCACACATGCTTATCTGAGAGATGAAAACACTGGGTTATTGGAGGGGGCTGTGAGAGCGAGCGAGCACAGCC
CGGGCATCTGTTTGAGGACACGACCCTAAGCAGACCCAGACGTATCAGCTCACACTGCTGCTGGCGCTGCAGGCGGTTGTGAGCCTCTCCAAAC
AACGCGGCCATGAACAACCAAATTCAGATCCACTGCAAGAGCAGCAGGCTCTTAACCAACAAGCATCTGTCCAGCCTGTAGACTCTAAGTTAGT
ATCATATGGAGTAGATGACACAACTCCATCTTGTGGATGGATATCAGATAATGTGATGACTTCCAGAAAGAGACTAAGAGTCCTGGACCGGCGC
TTGTGAGCTCTTTCAGAAGGCGAATGTAGCCAACCACCTCGTGATGGGGGAGAAGGGAGGTCAGAGGGACCACGTTTCAGGGATTGTCATAGGT
CAAACCATAAGTGACTCATGGGTTTGGAAACTTAAGGCAGTCAGAACCGTTAAGCAACCTTTATTTGAAGACTAGTAGAAGTACTTAACTTTCA
GAAGACTTCGATGTGAGAAGCTGTTTCTCCCTGGTGACTAGAGTTGTTCTTGAACGCTTGGGTGTAGAGAAAGGAATTCAAGCAACACTGGGTA
GACTCTTTTAAGAACCTTTCTCACAGACTTAGAAGAAGAAAAAAAAAAAAAAAACGTCAAATGAAACAAACAACCCCCCACCCCAAGCCAGCCC
TGTTTTCTTGGAGTGGCACCTGCCGCCATGTGTTTGAATAAATGATAGCCTTTACCATCATCAAGAAAGGGCTGCTATTTTTAGAATAAGAAAG
CAAAGGAACTATGTGGATAGCTAAGGGGAAGGGTCATTTAGATGCTTTGTAGGGGTGGAGAGTAATTAGGGGATCTAGAATAAATATTGTGGGT
AAAACAAGATGGCTTTTCCTGGAAAAGGGAAGAGACACAGTTCTCTGAATTAGTGACAAGAACAGGTCCTGAGCTTCAGTAACAGAAAGGAGAG
ACATTTGTGGAGGACCATTGTGGAGGCTTCTCGTTAGATCTCAGGAAATCCCAGCAGCAGATGTCTGTGACGGCAAGAAGAGATAAAGGAGAAT
TATTTTGCATCAAGAACTGTTCTAAGCAATTTAAATTATCTCTTCTTAATCCTTCTGAAAACTCAGAGACTATGACATTGAAAGTTAAACAACT
TTCCCAGAGCCTGATATCAGCTTGTTATGGAGGGTCTAGGATTCAGAGCTATTCTGTCTGACTCCAACGCCTGTGTTCTTATGGTGAGAGGACT
CACTAGTTTCAGGTCTTCGAAGTTCACCCCTATATCATACCACGATGAGAACTTTGACGGAGGTCCATCTGTCCATCAGAGAAGGATGCTCTAA
TATATTGGTTCTCAACCTACCTAATGCTGTGACTCTTTAATACAGTTCCTCATGTTGTGGTGACCCCCAACTATAAACTTATCTTTGTTGCTAC
TTCATTGCTGTAATTCTCCTACTGTTATGAATCATAATCTAAATATCTGATAGCAGGATGATCTTGGGTAACCCCTGTAAAAGGGTGGTTCAGC
CCCCCAAAGAGTCTCTACCACGGGTTGAAAACTGCTGCTCTCGTGAGAAATTGTGAGGCTGAGAAATTGGGGTGGAACAAAACTCTAGACCCTG
TAAGTCTGACAGAAGTTAAGATAAATGGACAGAAGACAATGTGAGGCAGATGCAGGAGACAAGAGGCCTCAGGTCAGCAGCCAACTGGAAACAG
GAGGGAAGGCAAACGAGGGTTCTGACTTCATGGGGACAGCTGTTGGCTTGACTTGACTTTCTGCTGGTGATGACTCAACAGTCTATTTCCACAG
CTGCTGTCATTTCTTTGGAGGTTTCGGTACCAATTCTGGTCTAGAGACAAGACCAGGGTCCTAGACACACAGTTAAGCTTAGGTTGTTCTCTGG
TATTTAATAAACAAGCTGTAACTCAAATAGCTTGTTGAGAGGCTGTGTTGGGATCCAGGTATCTTTAAAGGCAAAAGAAAAAGCATATATCACT
TTAATTTCTTCCTACCTAATATAACTGTTTACACTTTAGTAGTTTGCCTTCGATAAAGAACATTTAGTGCTATCTCAATTGTTAGTCACGCCCA
CGTTAACAAGGTAAAGTTACAGGAAGTACTGTGAGCACAAGGAATAGGAAGGACGGGTGAAAGGAACGCTTCTGTTCTTATAGTCCTTCCATTC
TCACATAGAAGATAGGACATTATTGATACAGGGGACATGTTACATACTTAACAGTTATTAACATACAGAGAAAAGATGAAGCAGGGCAGAGAGC
AGAAGATGGGGTGGTCGGAGCTTAGGTGGAGGAAGATTCGAAAAACACTTGTCTGAGAAGAGAGCTGTGCACAGACTGGAGCTGGACAGAAGAC
AGCAGACATCCTGGAACAATGTCCCAGCTAAGCACGCAAGTCCAAAAGCTCTCTGGTGGGAGCCCTCTTGGCAAGTTCTGCAACAGCAGGGTCT
ACTATAGATGCATTGGAAATACAAGAATCAAGGTTGAATCAGGACCCCACTCCCCCAAAACCCCCGAGGCTCCCTCAGCCCCTGTCCAGCCTTT
TTAAGAATAGAGACATTAAAATGTTTTATTTACTTCTGTTCTTTAACATGGGAAATTGTAACCATGTATTCCCAGATCTGTGTGTTGAGGCCCT
TGTGCTAATCACTCAATTAACATGGGAAATTGTCATGTATTCACACAACTGTGTGTTGAGCCCCTGTGCTAATCACCCAGTTCCAAGTGTTTTC
ATTTTTGGAATTTTGAACTGAGGAATGATGACTTAGCATGTCCTGACTTGAACAAAACTCACTCAGAACAGGTTCTGAGGAGGCAGGCAGGGAG
CCAGGGAGTAGCCTGAGAAGTGGTTATTTGGGGCTTCGTTTGAAGATCTGGCTTGATTTTATTTATTTTCTAATTTTTTTCAAAAGAAGAACTG
AAAAGGCAAATAGAACAGTGAAAAGAGAAAGGTAATAAAGAGGACCCAGCCCAGGGAGCACCCCTTCACACTTCACCTTCTACCTAGACGTGTA
CCTGCCTGGCAAAGGGCTGGCAGAGGGAATATTTCTAACACCATCCTATAAATGACAAGCCACATTCCTATGCTCTTATGGAATCATGGAGTAT
TAAGCCCCCACACAAAGGTGAGGATTTGTCACATGTCACCAAAGCATTGAGACCCAATTGTACCTGAGGTACCTAGGGGCGCTTGACCTCTAGA
TATTTATGATGCATGTGATAAGTTTCAAAATTAAAAGCAAACCAAAAAGAAAACAAGGAAGCCCTGCATCAAGGCTGATCAGGTGAAACTTTCA
GAACAGGGGTACATGCAGGTAGAGGTGTTTCTCTCTTCTCTTCTCTTCTCTTCTCTTCTCTTCTCTTCTCTTCTCTTCTCTTCTTTTTTCTCTT
CTCTCTTCTCTTCTCTTCTCTTCTCTTCTCTTCGTGTGTGTGTGTGTGTGTGTCTGTGTGTGTGTGTGTGTGTGTGTGTGTATAGACTAGAGTT
TATTCAGGGCCTGGGGAGGGGAGTTAAGAGGGTACTGGAGTCAGAGAAAGGCAGAGAGAGGGAGAGAGTAGAGAAGTAGTTGCTGGCCATGACC
ATGTGGAGAGAGAGGAGAAGGGAATGGGGAGAGAAGGGGACCAAGAGGGAGAGGCAAGAGAGCAAGGCAAGAGTGAGGTGTTACCCTCTTTCAG
AGCAGGATGTCTGCAGGTAGAGGTATTACCTTCTTTCAGACCACAGGTGTGGGCAGTTAGAGGTGTTACCCTCTTTCAGGGCAGGGGTGTAGGC
AGATAGAGCTTTTACTTGCCTGCTCTATATGAAACCCTCCTATCTGAGTGGTGGTTTGCCATTGTAGTTCCAGCCTGACTCTTGAGATGTTGAT
CACACTAGTAGGGAGTGTCAGGAACAGTGCAAATCCATGAACCCTGGTGAAGGTCCAAGTGTGCCAGCAATAGTGGTGAATCAAAGAGGTTTTC
ATGTATCCTGTCCCTGCTTTGCATTCCCACACCAATATTCAGAGGGCTAAGAGTGAACTCCACAGTGTTCTGCATCTTCGCTGTCTCTCCAAAG
CTTACATCACTTGCTCAGGCTGTCTAAACTTGCCTTCTTATCTGCAAGTTAGGGTACTACTAGTGCCAACCCTGCCTATCTCAGGGTCATCAGA
AAGCCCAGATGTTCTCCATAGATAAGAGGGAAGGACAAANNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNCAAGACCGACGAGACCAGTGTC
ATGAGTCCTTCCTATGCCATTAGCTATAATCCCGTTTAGCAGGCTTGATCTGTGCTTCCTGGTTGCACAAATAAATAATAAAGAACAGTAAACT
GACTCCCTGTCATCCTTGAGAGGACATACATTATTAAACCAATATACTATTTCGTCTCTCTCCCTCCCTCCCTCCTTTCCTTCTTTATAATTTG
ACTTGACTCAAGAAGAGTAAGCAAGTAGCCTTTTTTTTTTTTTTTCTTTAAATGGAAAAGACTTTCAGGATGACAAGGGCACTCTTGCTCCTCC
TTTTCACACCTAGCATGACCCCTTTCTCACAATATGCCTCACCAGTGCCAACAAACAAGCAGGACTCTCTGTAGCTGTACCTCTGCTCAGATCG
GTGATTACACCCATGGCGCATTAAAGCCAGTTAGAGAGAGAAAAATAACCGAGCCGAGTGTTGACATGCTGCCGTGGAAATGTGAATAGACTTT
GGGGAGGCAATGGCAGACCCCGAGTTCAGGGCACCACCACCTTACCCCAAACCATTGGAACCAGGTGGTGCATGTTACTTGAGCTTGCAAAGGG
CTGGTTCTTTCCCCGGTGATATGGGGTGCCACTACTGACCCCACATCGCTATTGTGGGGATTTCTCCCTCCTCGGCTGTAGAGACAGAATATGC
ACATAGACTTAGATGAGCACGTGCTCTGGGAAATGAACAAGCATGCGAAGTAAGGCGTTATTTAACTCTTACTAGCCAGTCACATATCACTTTC
CTACCCTATGTCCTCCAGTCATTCTCGAAAAGGGCACAGTCGTGGACCCATGATGGGCTGACATTGTGAGGAACGTGGGAAACACAAGACACAC
GGGCTATTGGTGCAGCTGCTTGAAGGAACTCCTCCTGTGCGCTGTTAATGAGCAGGATGAGCGCTCGAGAGCACTGAGCACCCTGCGAGCATGA
CTGCGCTCGACCTGCTGAGAATGCCTTTCGAGCATGCGCTCTACCCGCCCGCAGAGAACGCCTGCCAAGCTGCCTCATCCTCCACTTGCTCCTT
GCTTACTCAGCTCAACTCTGCCTTCAAATCTCAGTCCGCATGAGTTAAACACACGGAAGCTTTCTCTCACTTCCCAACCGTGCCAGACACTGTT
ATTAGACATTCTGTTTGATGAGTTCTCCAGTTAGGTTTTACCTGTATTGTTGAAGTTATCCAGTTAGCGTCAGTCCCACTGTTTAATTCCGTGA
GAGAAAGAAATTGGGCTTGTTCTGAACGAGGTGTAGCCCCCACTCTCTGACCTTCTACAGTGTGGGGCTCTTAGAAAGGGGCGTAACAAACTAA
ACTGGTAAACTTTAGTACTCAGTGAAATAATGGGCTAGATAAGAAACAATTTTCTGAGATTGTTGATATCATCAGGAACCAAGTGTTGTCTTAA
CTCCTTTAATCTTAAAACAGTCCGGTGATACTTCGTCATTAATTTGATTTTTGAACCTGGAGAAGCTGACACACGTCACTCCACGACAGCTGGG
GGATTTGAGCCTGGCTTTGAGAGATCTTAACCTAACCATGACCTGCTGTCGTAAAGAGCAAGGTTGGGTTCACAAGCAGAGCCAGGCCCTGTTA
GAACCCTGAGGATGTTCGAAAGAAAAAATCAGCACAAAAGAATGATTTTAGGTATGATGTCTTACATTTATGCTGCTTCTGCTTTCTGTCAGAG
TACGTTAGCCGAATTCACCCAGAGAGCAGCCTCGCTTTTGAAACTGTGATTCTGACGAAGTTCTACGCCACTCATCTCTTGTTACCCCTTTGAA
GAAACAGCAAATGCTTTTCCACAGTAGGTCAGGGCTATCTGAGGTTAAAAAAAAAAAAAAGTACGTGGCTGACTAAGAGGGCTATACCTGAGCC
CATGACTTGTAGTTTTTATTCTAAAAGGAGCTCTGACCCATGGACCCAGAATTCTAGAAGAGTTTTCCTTCATCAGGGAAGGCCAAGGAAGTCT
GATTGTCCCCAAGAAGGGCAGCTGACACTAGAATAACATAGTATTTTGTAGAAAAATAAAACATACCAAAAGAAAGAAAATAAATGTATTTGTA
GAAAACTAACAAAACACTGCAATGTAGGAAAAAAGAAAAACCTTCCCCTTGGAATTGATGTGGGCCAAGTATCTTCCAGCCACTTGCTTCAAAT
GGACCTATTAGACATCCTCTAAGTGAGGGAGCATCTGACAGTCTGACATAGCCCCTGAGGGTTGTCCTCTCCTCTGCTGAGTACTCTTGTGAAG
TGTTTGTGTACTTGAATGTATATATTCCTCATTAACAGCCCCTTGAACATTACTGAACAGGTGAAGAGACCAAGCAAGGTCACACAGACTGGAA
CTGCCTCTCGCCCCCAGCCAGCCTAGCCTGTCTCTGAAGTCCACACTCTTCCCATTCCGAGCAGCTGACTATCCAGGATGGCTCCTTTCGCTTC
TTACCTGTGCTTGAGTGCTTACAATTAACAGTCTCTGTTCAGATTCTACTCTCAGAAACACCTTCCTAGTAGACAGTTGCCCTCTATAAACTTT
ATCTTCAAGAGACGTAATACTTCCATTCCCTTCCCAGAATAAGTTTCTACATTGGTGTTGAATACCTCTTGGCCACTATGGCATGATAAATGAT
GGTTCCAAGAAATCGTTGTCAAGGTTTGCCTGTGCCGCACAGCTGTCCCCACAAGTAAGGCTTTCTTTCTCTCAGATGGCATCTTGGAAAGTAC
AGAAAGCCCAGGGCCCTCCCTGTTTTCATTCTGTGGTCTGTGTAAAAACCAAATGCTACCATCCCGACTACACTCTTTCAGATCGATCAAACAG
CACATTGGTCACTGGCAGATCTTTCAGGTCCCTCACCTCAGAGTCCTCCCAGTTCCTCATTCTTGCCGCCCTTCAACCATGAAGTTAGTCGTAA
GAAAAACAGTAAGTGCTTCTTGAATCCACCTCCCACCTCTTTCCATCAGCGCCAGTCACTAACTAGCCTGTCAGCGCAGCTTCAGAAGGCTCGG
ATTCCAAACTCTCAGGCCTTCTCCCATGAGGCCTTTCTGCTTAAGTGGAAAGCCTGCCTGATTTCACCCAAGTGAAGCGCTTCCTTGAGCCTTC
CTGTGCTGTGTTCCTGTGCACACTGTCAGTTAAAACTGGGATCACGTGACTCGCTGCCTCTGCCGATCATTAGCAGACCGATGTGATCAGCCAT
CCAGAGCTCGAATCAGCCATCCAGGGACTTTGTAGGAACACCATCATCAGCCTTTGAGCAAAACCAGAACTTGGTATTCATTCTCCTACACTTA
AATGGGGAAGTATTTGGCCTGTGAAGAAAGACGACTTGCTAAATTCACTTCATTTTAATATCAGAGAGAGTTTTAAGTTCTGTGCCTAAGAAAG
CAACTCATCCTTTGGGACAGTCAAGGTTAAACTTAGTACAAAACCTTTCTCTGAATTACGTCACTCTGTAACTATCCGAAGGCAGTCCTCCTGA
TGGCCTCGCCGCTCTGGAAGAAGTATGCCCTGGTCTGTTTTATCTTCTGTTGGTTGACAAGCAAATTACACAGGATTAAAGTTTGCTTGGGCTG
GGGTTTGAAGCCTGGCAGGGTTCAGGTTGTTGGTCAGCCATATCTCACAGGAACTTTGCAGCCCACCACAAGACAGGAAATTACATAACAAGAG
AGGCCATCTTGGCTGAAGTATCTCTTCCTCTTTGTATAGCTCACAGCTCCGTAGATGAGGGCTGGCTCTTATGGTGTATTCGATTTTACCTCCT
AAAGATCAACTTCCAGACACCATCACTGCATTACATTTCTAGGCTCAAAACGTCTCATCAGTTTCGGGGTGGGTTCAGAAGCAACTCATTATAA
TGTTGTATCAGGGCTCAGGCAGGCAGGCCTCCTTGGGGAGTCTAGAGAGCTGCCTCGCTGGCCAGAAGGGAGACCCTTCGTTGTTCAGGGCAAT
GGTATCTTTTCTCCATACTCTCTGGGGAGTTACGCGAGCAGTGATCAGCTTCCCGGCTCTGACCATCAAAGGTTACACTGGACAAAATCCATAT
TGAGAGCAAACTCTGTAGAGCCCTGCCTCCGTGGAGTCTGGACCCCGCTGATAGTAGTGATATTATAAGTGAGCAGGCTGAGGAAGGCTGTGTG
TCTTTACAGGAAGCTCCTTTAGCAGATGCAGGCTCTTCACGTTCACGTTGACATTTATCCAGAGAGGCGACGTACCTCATGCATTTCTAACAAT
AGGTGGGATTTATGGCAGGTTCTGACTTAATCGAATTCAGAGGGGAGTAATGCCTCCGAGCTACTCATGGTGTGAGTCTCATGGTGTGATTTCC
ACGGTGTGATTCTCATGGTGTGGTTCTCATGTTGGGCCATCTTGACGAAAAGTTCTACACTGTAGAGGTCGGGATGGTCAACTAGACAAGAAAG
ACTTGGTCATCTCCTCTCTCCTGTCCTGTCCATTGCTGTTCTTACCACGTTACAAATACACAAGTGAAATGATCGCCTCACATGGAAAACTTAA
CTTTGTAAAACAAGAGCTAAACAACCAAATGTGAAACCTACAAATTTTTAGTCAGAAAGTTTGCTTTTTTTATTTAAGAAACTCACAGACTCCT
GAACATATACAGTATAATGTCAGAACCCATGCCTGGGGAAACTACTGAAGAGTAGTGGGTGTCCTGTTAGGTGATGTGGACCAAGCCATGGGCT
TATGTCATTAAAAACAGCCTCCAGAGCCAAGCCCCTGAACAAGACTACCTTTCTGGTAATCACATGACAAGGAGTTAGGACTCAGTCTTTGTTT
GTCTCTCTTACTTCATCAGTCACCCATTCCCTTCTCGTGCATGGTTTGCCCAGTTTTCATCACTGAAAATCGTGACAAGGTTTCAGAGAAGAAA
CAGTGTTGGTTAGTAAGGACCCTTGTGACAGTCCTACCTAATGAGGCATACTCTGTTTTCAGTGACTTGTTTCCTGGACATATAAACAACCCCC
ACTACCATTTTGATTTATTTCCATCCTGTCATGTGTTCATTGTATATTTTTTAAGTTGAGCTATAGTCAAATATGTATATATCTGCAAAATGTT
GTATTTATGCTGATTGGTTCCATGTTACAGTCAGGTCAGATGTACTAACTGTTGAAAATGTGCTTCTCATATAGATTTGCAACTATGTGGGGCC
TGCAAAGGTTATCGTTCAGTTGGTCACAAATGCAAAAAACATCCACCTGCACGCCCACAGCCTGGTCGGCAAGCACTGTGAGGACGGGGTATGC
ACCGTAACAGCAGGACCCAAGGACATGGTGGTTGGGTAAGTGGGCTGTGTGGTATGGAGGGAGCGGGAACACGCGGGTGTGAAATCCATAGGCC
CCTTTCCTTGTGGACCTGTGCAAAGCCTGAGAGGGTCTCACACACGCGCAACCAGATAAAGCCAGGGGAATGTGTGTCCTCTCCTTTGAACTGC
TAAGGAAATAGAGGTGACCAGATATCTGTCCTGCCTGAGACAATCCTTGCATCTGTAGTCTTGGCCTCTCAAGGATGGGTTTGGACAATGTTAT
AATTATAAGCCCTTGACCTAAGAGGCATTTGTGCCACTTTCAATTATATTTTTCTTCTCCTGGAATTAAAGATGAAGAAAAGAATGAAACAACT
ACCTTAACTATATAGAAATGATTACCATGGAGATGCTTTTTCCTTCCTTGAAACAGCATCTCCACTATAGTTTAAGCTTACCTTGAACTCACAT
AGATCCTGTTGTCTCTACCTCCCAAGTGTTTGGGTGCTCGGATTAGAGATGTGTACCCCCCCACACACACACAGAGCTCTGATTTTTGCCTATT
TGGGATTTTCTTAATAGAGTTCTAGTGGGAACATTTGAGACATGAAGCAGATAAGTAAATATTGTTTCAACTCTGCGCTACTTATAAAATGTGT
GCTTTTTAAGTCAACAAGACATGCAAATTGGTTTGGGCACTCATTTGATCTGGAACATAAGTAGAAATTAGTGTATTCTTTCAAAGTCAGTCAA
ATACAACAATTTTTAGTGAATGAAATTTTATAGAAGTAATCTTACTTCAAAAGAATCACAAAATATTTAATAAGACATAATTATGGGCTGAGAG
AGGATGAGTTTAGTTTGCAAGTATGATGGCCCAAGTTCAAACCCCTGCCCTCTCATAGCAGCGCTTTTCAGAGCACAGGCATTTATCCTCCCAG
CTCCTGCAGGGGAGCAAGACGCAAAGACACGAGAATCTGGAATCTCATGCAGTAGCTGGCCTGCTGTGGTGGACAGACACCAAACAGATCCCAT
GTCAAACCTGAAGTTCTCCTCAGACCTCCATATGCACAGCACATCACATAGTCAGCTCCGTTCGCACACATAAAATAAGGGCACTCACTATGCT
AAGGTTTTATATTTCATTATCCTGAAACACTAAAATAGCAGACAATGTAGTAACTTAAACAGCAAAAAATGATGTAAGCATTCAGTCATCTCAA
ATACAGATAACTCCTATTTTCCAGACTCTTAAAAAGCAAAGGGGATGATTCAGTGGGTAAGTGCACTTTCCACCAAATCTGAGAGCCTGAGTCC
AACTCCTGTAAGCCACATGGCAGGAGAGAACCAACTTCCACAAATTATTCTCCAACCTCCACATGCAACAAAGGTATAAGTGCACACACACACC
ATGTAATATAATTTTTAAATAAAAAAAAGTTAACTAAAATATTTTTAAAGCCAGAAGGTAAGAACTGGGGAGACAGGTCAGTGGGTAAAGTATT
TGCAATGCAAACTTCAGCGCCTGAGTTCTGAGCTCCAGCGCCCACATGAAAATCCAGGTGTTAAAAAAACCAAACTGATAAATAAAATAAAACC
GGGAAAGGGCAGAGCACAGCGGTGCATGCCTTTAATCCTAGCACTCAGGAGGCAGAGTCTAAGGCCAGCCTGGTCTACAGAGTGAGTTCCAGGA
CCGCCAGGGCTACACAGAGAGAGTCTGTCTCACTGCCCCCCAAAGCCAGGTGTAGTGATGCACCTGGAACCCCAATGTCTGGCCACAGGGATGG
AGCAGATCCGCGGGGCTCACAAGCCAGTCAGAAAGCACGGCCCAGTCCATGACCCTGTCTCAAAAACCCGAGAAGTGACTGAGGAAAATTCCCT
GCCTGTGTTCTCTGTGGTCCATGCAGATACATTGTTGTTAATTTTATTTAATTGTGTACATCATCATTCAGAGTTCTCCACTCTTACATCAAGT
AGTGTCTGGGAAGACCCCAGTGAATGTGTGCTTTTTTTAGGAGATTGCTTAGCCAGCCTTGATGAAGTCTGACTCTGTTGCCCTGCAATCTTGG
GAGGTTCAGCTTTCAAAGTCCCAGGCATCATTTCTTCATTTGACGACGAAGATGCTCAAGTGTGAGAAGCTCCCAGAGAGAGGGCCCTTCCCTT
TAAGTCATTCATCTCTGCTGCTTTTTCACGTTAGGGAGAAAAGTATCCGAATTGGAGGAAGCTCAAGTTAGTTCTCGTCGACTTCAGCTTGTCT
CGGCATCTCCTGGCTGCCCTGCTTCTCAATCTGCCGAAACTGACATGCAGGGTGACTGTCAGTTTTAGACAACTGATCGGTTAGTGTATTCCCT
GATTGATTGGCCACATCATAGCCAATCGTTTTCAACCATTTCCGTTCTTTTCGTCTTGCTTAATCTCTCTTACAATCATATGTTCTTAAGGAAA
TCACAGGTGAACTTTTCTATCAGTATTTCTGTCAGTAATATTTATTATTATAAAACTTGCAGAGTGTTGCTTCTCTGTTGGGTTTAATAATCTT
TCATGATTTAGAAGAAACTAATCGTGGCTGTGTCTGCTGCTCTGAGGTAGACACAAGGCCTTGGGTTGCTCTCCCCTGCAGCACTACCGCCACT
AAAAACACTAATGGCTGTTTATGCTGGAGCCTAGGTTGCAGCGATGGAGGTAAATTCAGTACCTGTAAGCAGAGCGGAGAGGTCCGTCGGTGTC
TTTTAGTGTAGTGGGGCACGCGGCTCTAGAGTTGGGAGTGTGCATTACTCATGATTTAGCAAACATTTTCGACATCCGATTCCTGACTAGGCTC
CCTGTGGTTTGATCTAGTCATAGCTGGGTGGGATTTTCTAAGGTGAGGAAACTTTCTCGGAGAGATGCAGTCGAGGAGATGCCGCCTGAGTGAT
AGGGGGTAACCACAGTAGCTTTTCCCACCCAAGAATCTCTCACTGTAGAATGTCTGCAGTCGCTTTCCCGGTTGGCATTTAGCTGAGAGAATTC
TGCTCATGGTATTTTCCCCAGACACCATTTTCTTCGTGATTCCCTTGTCTCTGTGTCTCTGAGTTTCTAACACGGTCCTACACCTTTTGCCTTT
TCTTCTGTTTCTCTTCGATGGAAAAGCAGCGTCCTAGCAGCCGGGAAGTCAGACCTCCTCTATGCTTCTGTCACTCTGCACGCCAGAAGGGATG
GAGGCCAACTTGCATCACTTTCTAAAGACTTGAATTCCTTTCCTTACAAAATCAGAAAGACACAGCCTCTCTCTATCTCCAGGCACTCAGTGTT
TAACCCTCTAGACCAAGCCCACATTAGCTAATGTTATAAAACAGCAGGAGGAAACTTGCCACTATCTCCTCATGGTATGACATCGCTTATTTTA
TTTAGTTCTCACTTGTAGGCCACAAGTTTAGGAAGGCAGCCAAAGGTGTTTCTTAGACTTGTCAATACTAGAAATAGAACCAAGATAACATTTT
CCAATACTACAGATTAGACTGAATATAGATTCACAAACAAGGCACCAGTCCAAACTATCAACATATGTAAAAATGACCCCAAGAAGACCAAGCT
TTGAACACATCAAAACCAAACCGACCCCCTTTGTTTACAGTGTCTTTCACACTTTCTTCTCTGTTTGAATTCTTAATAAGTTTAGTTTCTATTT
TTCCAAATTATTTTATCAGTATTAGAACTATCATCTCTTGGGGTTTGATTATGGATTTGATTGTTTTAATATAGGCTCATTAACTTCAGTTAGC
TTTCATTAGACCTCTTGTCTCTTATGCACTTAGTTAGACTTCACTGCCTTTCTTAATAAAAAAAAGAAGAAGAATCCTGTTTAGTATTCTATTT
TGACCAGAATGTTAATATTTTTAGCAACACTCTAGTTCTCTGAAATTTTTTGTTCAGTGGAAAATGTCTGTGCATTTCTACTTTGCTGTCGGCC
ACGTCCACCAAAAGTCACCACACCCTGTTTGCTTACTGGCAACCTGGGTTGAAAGAGATGTACAGTTCTTTTTATTTCCCCCTCCCAACTAATT
TATGTAGCTTATTGCAGTTGGTAAGCAGGAGAGCAGAGATTCTCAACCTGTCGGTTGCAACCCCTTTGGGCGTCAAATCGTCTTTTCACAGAGC
ATGCCTATGGACATTTATATTAAGCTTTATAACAGTGGCAAAATTACAGTTATGAAGCAGCAATGGAATAATTTTATGGTTGGAGGTCACCACA
GCATGAGGAACTGTTAAAGTGTTGAAGCATGAGGAAGGCTGAGAGCCACTGCCCTGGATGATAGTAGATATGAGAACACACAGGAGAGCCTTTG
GGCTCCCTCAGGAGAACTGCTACCTTAAATTACTGCTGGACAGATCCAGTGTAATTGTGTAGCTAAACACTAGCCCACAGGAGAGGCACTGCCT
AGAGAAGGTTGGACAAATATGGACTTAGGTTCAAGGTGTGCCGTACCAAGTTGGTGTATGCACTGTGAGACGAGAGCTGCTTTTGAACACTGAA
ATCAAGGTCTGAAACAACAAACGCCAAAGAGGGCCCACTGTGAGCCCCAACCCAGTGTTAGACACATCAAAATTTGAGCAACAGCTTCCAAATG
ATGATGAAGATGAAACATCTCACCTACTTCACCATCTTTATTCATCCCTACAGGTTTCACTCAGCCTCCGTCACTTCATAGAAAACTGATGCCA
GCCTCCATCCCATAGAGTGGGATTTCACAGAGCAGTGAAAAGCCAGGCCCTCCAGCCTCAGCATTCCTCTTGAGTTAATGGAAAGGTTTGTTTG
AAAAGTCCTGATTCCGTAGGGTTCGATGGACCCCCACATTTTTAGTAAATTCTAAAACAACACTGATCTTGCTACTTAAACACTCCCATGTTGT
GAATATGAGACGAAGAAGCAAGGTTGTGGTTTCAGCCCTTTTATTAGTCGTTGTAGCCTTTCTGTTTTTAAAATCTCATTGCTCCAACACTCCT
CCTCTCCAGTGTTCATTTCTCTGAATTTTGATAATCATGTGGCTACTCCTGATCTGACGCTGATCAGTTCTCTTTGGTTTTATGTCTCAAAGTC
GTCCCTTCATCTTCTCTGTTCAAAGGTGTTTTTTGAAGAGCAGAGGATTCTAAGTTGACAATATGTACTTTCATGTGCTGGTTTTATGTTGATT
CACCGTCTCAGGTCTTGACCTGTTTCCAGCAAGAGCTCTCTGTAGGTCTGCCTCGGCTCTACTGTTCCCAGAGTGTGATTGTTTCTCTAGCAGC
GTCTTGACTTCTCTTTAGCACTCGTAAGCACTGTGAGTTTTGATGGGTTTGAGCGTAGCTTTCTTTTTAAGCTTGAGCTTCTTAGTCGTCATAG
ATCCTTGGATTTACATTTCTTATTAAATCTTAAACCTTTTCAACTGTTAAGTTTTCTGTCACTGTCACTGCCCCTGCTCCCCATTCCTGTTCTC
TGTCACTTTCTCTCTTCCAGACTAGGGTTGAGTTGGCACAGTGACTAAGAGAATACCTTCTGAATAGTCAATCTGATGCCTCATGTATTCCGAG
GCTTCCTGTTTATGAGAATAGTAGCTCTTTCCCTTAGCCTAACAGGGCTTTCTCACAAATGTTTCCTTTGGATTTTTGAAAATCTGTGATGCTC
TGGATATAAATCCTAATCTTCTTAACCTCAAGTCTTTGCAACTGCAGAGCCCTAGCTGGCTGCCTGGGTTCTCCTTCCCATCCTGCAACCTAAG
CATTAAGCCAGGTAATTGTAAGAGTACCTTTCTATTTTTTTTTCTCGGATGACTGTTGTCTGTCACCTATCTCCAGACCATGACAATCGTTTTC
TCGTGTGTTTTGGCTATTTAATTCTAGAGGAACCGTCTAGGTCTATATTACTCCACCATGGCCTGTGGTGGAACACTGGTGTGTTGTTTACTAG
GGTAAACCCTTCCTTCATGATTCATATAATAAATAAGCATGATGCCTTTCTCCAGATGTGAAGCTGGGTTACGTGTAAGAAGTGCAGCCGAGGT
GATGGTCACTCATGTTCCCAGGAGATGAGCCTGGGCTCCTAGCGGGGGAGCAGTCTGCACAAGAGTGAGACCCAAAAGGGCAGACACAAGATCT
GTTAGACACAAGGACCTCCAAGCACATTACCACGTTGGGTGCATAATGTTTGTGATGCTCCCTCCCTTCAAGCTTTCCTGTGGTCAGCAGGCAC
CTGCTGAGGACCCACATGTCAGCCTGCATTCTAAGACCTGGGGATCTCTGTCCTGAAGGAGCTGTGGAAACACAGCATGTTGACTTCTCTGTGT
CCCTGTAACTCACTCCACATCTCGCCTTCTGTCTGTGTCTGTTTCCTTCTGCCTTCACCTCTTCTCCTCCCTTTCTCCAGTTTATATGACCTTT
GCACAGTAGCTTCTTCTCATGTGTGCAGGCAAGTTGTCAGATCTCTTTTATTGTCATTTTATATTACATTTACCGTGTGTGTGTGTGTGTGTGT
GTGTGTGTGTGTGTGTATGTGTATTTCTGCTTGTCGGGGGAGGCTATCTGTTCTCTCTCTCTCTCTCTCTCTCTCTCTCTCTCTCTCTCTCTCT
CTGTGTGTGTGTGTGTGTGTGTGTGTGTGTGTGTGTGTGTGTGTGTGTCAGTGTCAAAGCCCAGAACAACTTGTGGAAGCCGCTTCTCTTCCCC
TCCTATGTGGGTTCTACGGATTGAACTCATGTCACCAAGCTTGGCAGCTAGTGTTCTTACTGACTTAGCCATCTCACTGACCTGTATCATTTTC
TTCCTTCTACTAATATTTATTTTACTTTTAGAGTAAAAGTCCTTACAAACATCTATATGGCTCTGAGAGACCCAGGCCCTGCACTTTTCTCTTT
GAGTTCATCTGTCCCCTCTGACTCTGCTCCTTACCCTCAGCCCATGTGCCACCTTTCTGCCACTGTCAAAAACAGCAGAGTGGCCCTGCCATGC
GGTCTTCACATTCACCAGAGTGGTCCTGCCATGAGGTCCTCACACTCACCTGTCTGCAGCTCTGATACTTAGAATCCACTTCTTCTGACCTCCC
TCAGGACTGGCTCAGCTGTGATCTCAGGGTCCTGTCTGGGTTCTCCCTGTCTGGCTCTCCTACCCTCTCACCCCTCCCAGGCCTGTCTTCTTTT
TCTTGTTTACATGTATTTCTCTTTTAAGTTACACTGTGAAATCTATCATCGTGTACGCTAGCATGTGCATTGTGTGAGAGCACAGTGAAGCCAC
ACAAGCCTTCCCTAGTAAAGCATACGTACAAGGGCACCCGTACTCTGTGACTCCTGAGTTGACATCTAATATGTTCTGAGACGTAAGGTGATTC
AAGTTTTCCTCCAACTCTTTTAGTAACCCAGTTTAACCTGGTCTGACAAATCCACTTCTCTGATCAGTCTCGTTAGACGCCTCTCTGCAGAAAT
GTCAATCAGCTGAGTCCATGGTGCACCCCAGCCCTGCCAACAGTGTCTAGTGACGTACAGCATATGCCATATATCACCCTCATAAAGTCCCAAA
TTCTGATGTTCATCTGACTTTGGTCTCTCTAAGATGCTGAGTGCTTGTGTACTTTTTTCGGTGTGCTGTCTGCTGACCTGAAGCCCGCCCCCCG
GCCATGACCTGCCTGACTTCTCCTCTCACTGTTTTTCTCCAGCTTTGCAAACCTGGGAATACTTCATGTCACTAAGAAAAACGTATTTGAAACA
CTGGAAGCACGCATGACAGAGGCGTGTATTAGGGGCTATAATCCTGCACTTCTGGTCCATTCTGACCTTGCCTATCTACAAGCACAAGGCGGAG
GAGACCGGCAACTCACAGCTGAGCCAGCTGACCCAGCCTTAATTAAGGCTTCTATGTTCCACGATCACTCCACCCATCACCCTGCCACGCTATC
AGAGGGAACGGGATCAGTGCAGCCCGGTCTTTCCTTTCTGTGCTGGGCACCGTGGAGAAGCCGAGCCCACACACAAAGACTCTGCATGCTTGTT
CTCACCTATGTCTGCGCAGCCGGCGGTGCAGAGACGTGCATTTCCCTGTACTCCTCGCATTCTATCTGTGCCTTAACTCTTAGCAAGCGTAGGA
GAGTACATCGGTGAGCATGGGAGAAGACAATATTTACAATACTCCAGAGAAAACGCAGCTTACCTTTGATGGGTTTTTAGGCAATTTAATAAGA
CCATTGTGTAAAATTTTACTAACAGTTTAAAGAATTTCAGGCCAAAGTAACCAAAAGAAAAAAGAAGACTCAGTTATTAAAATTAAAGGTGAAA
ATCAGGGAAACATTACCAGAGGTAATGGTCAAATCCAAAGAATCATTAGGGCTTTGAAAATATGTATTACAGCAAATTAGAATATCTCACAGAA
ATGGGTAAGTTCACAGGTACATCTGACCTGCCAGTGTTAAACTAAGAAGATAGAAACAACTTACACAGACCTAAGACAAGCAATAAAATATCTC
CCAACTGAAAAATACCCGGTCCTAAATGGAATCATGGCTGAATTCTGTCAGATATTTCAAGAGATAACGTAAATCCTCCTTAAACTGTTCCATA
AAGTAGAAAGGGGAAGAATATCAACAAACTTATTTTCCAAAGCCAGCATTACTTTTATACCCAAACCAGGTAAAAGAAGAAAAGAAAAGAAAAG
AAAAGAAAAGAAAAGAAAAGAAAAGAAAAGAAAAGAGAAAAGGAAGGAAAGGATAAGCCGATTCATACATTCCCAATAAAATAATAGAATGAAA
TGAAAAATATACAAACGGGAAAGGAAGAGATGAAAGAGCCCTATCCACAGACAATATTGTCCCACACACAACACCCCAGGGCTGCACTGGAGAG
GCTTAGCTCTGATTGAGGATCAGCCCTGCGTCATCACCACACAGACCCCGTGGTTTTCCTATATCGATAGTGAGTGTGTCGAGAAAGAAGTCAG
CAAACGCGACGGCCCAGCACAGTCACACGCATCCCCCATAGCTTCAACATGAGCAAATAAAACTCCTCGCAATAAACTAACCATGTGAAAAGTC
CTTTACAGTGAACATGTTAAGACACCGAAGAAGAAAGAGATGTCAATCAATGATGGAAAGGCCTCCCATGTTCCTGGCAGTTAACAGAGTTACT
ACCACAAAAATATGGCCAAGTGTGGTCTGTAGATTGAATGCAATCCACAATGCCAATACCGTTCCTCCTAGAAAATAAAGTTCACTTGGGACGA
CAAAAGGTCTCAGGTAGCCAAAGCTATAGGATAGTAAGAATATTTCAGAGGTATCACAGCTGCTGATATCAGAGGTATCATAGCTGCTGATTCC
AGGTCACACTACACAGCCATGGTAACAAAAAAAGCATGGAAGTGGGGTCAAAACAGACGTGTAAATCAGCAGAAGAGGAGAGTCAGAAAGGAAC
ACAATGTAGCCACCTGATACCCGAGAAAGATGCCAAAATGTGCATTAGAGAAAAGACGGGCTGGAGAAGAAGTGATGCCGGTGGCGAACCGGCT
AGCACATATAAGGAAGGGAACCAGATCCCAGGCTCCTACCCTACCAACCAATTCAACACGGAGCAGAGATCTTAATGGAAAAGGTAATGCTCTG
AAACAGCTAAAGGAAAACATGCAGAAAACGCTTCGTGATGTAGGCAAGCCCTTTCCGAAAAGGGCTCCAGCAACTCAGGAAATAAAATGGAGAA
CTAGCAACTGAGATTCAAAGGCGTCACAGCAGACGAAACAGTTTCCTGAGTTGCAGAGACGACAGCCTTCAGTGGGGCAAAGAATTGTTCCTAG
CTATGTAGCTGACAGATTAGTATCTAGGTTCTATAAAAACTCAAAAAAAATTAAATTCTCCAAACAAACAACCCAGTAAATAAATGCGCTCAGA
AGAAAGAGAGGGTTCTCCAACAATGAGTGCAAACAGCCAACAAATGTGAAAGCATGCTTCACACCCTTAGTCTTTAAAGTTCCTCTGAGGTCCC
ACCTCACCCTGGTCAGAAGTCATTAAGAAAACAAGTCCAGGAGCCTGCCAAGTAGCTCAGGTGGTACAGTACTTACCTCTTGTGTAAGAAGTCC
TGGGTTTGATTCCCAGCACTCCCTACAAACGGCTGCACTCCCAGATGAGTTCAAGGTCGTCCCCAGATGTGTGTAGCAGATGTCTTAGCAGGTA
ACCCATACAAAGCCTGCTGCCCTGAATTCAGTCTCCAGGACCCACATGGTAGAAGGAGAGAACCCCTCCCCTCAGGTGGTCCTCTACCCTAAAC
ACACGTGTGTTATGACCAACAAATGCAAGAATGTAAATAAGAAAATCAAAATGGAGATTTAGAATAACATGAAGTAAAACTGCATAGACACAGC
TATCCACTCCTGCGGATATTTACAAAACTCTAACTCCACGTACTATAACATACATATTTCCCTGCACATATGTGTTTATTTCAGCCATATTCAA
GAGCTACATCAAACAACCACCGTAGCTCTCCATTAGCAGATGATGGATTAAGAAAGCATGATATACATGAGTAATGGAGTTTTTTTTTCAGCCA
TTAAAAAAAAAAAAAGAAAAAAGAAAGAAGGGCTGGAGAGATGGCTCAGCAGTTAAGAGCACTGACTGCTCTTCCACAGGTCCTGAGTTCAAAT
CCCAGCAACCACATGGTGGCTCACAACCATCTGTCATGGAACTCGATACCCTCTTCTCTTGTGTCTGAAGACAGCTATAGTCTACTCACATACA
TAAAATAAAAATAAATCTTTATTTTTAAAAAAGAAGGGAAGAAAAAGAAAAGAAACATTTGCAGGGCAGTGTATGCAACTGGAGATCTCTGTAT
CAAGAGAAATAAGCCAGAACAAAGAAGATGAACATCACCTACCGTCTCTCTCGGTTCTAGATCCTAGGTTTTATATAGATGTACATAAGATATT
TTGCATATCCGTGCATAGGATATGAAAGTAAGAGGAAGGTACTCCGTTGGTAGGGAGGGGACGAGTGGATATGAGGAGGGGAGAAGGGGCCTTA
GGTACGGTCACAGTGCTTGTTCTACGATCAAAATGTTTTCAGGGAGTTATCCCGATGAGCACTGAGTATATGCCAAGAAAGACTTTAACGAAGT
GTTTCTAGAGGAAAAGAATTGGTCTTTGAGGCCATTTCCTGTCAAATGTTCACACTCTGACCTGCCTGGGCATGAGCAGACTGTTCTCTGTGAC
GCTAGACAGAGAGAAGGAGATCATCCGCCAGCCAGCCGTGCAGCAGACCAAGCAGATGCACCTGAGCCTGGTGCGCCTCATGTTCACAGCCTTC
CTCCCTGACAGCACTGGCAGCTTCACTCGGAGACTGGAGCCTGTGGTGTCAGACGCCATCTATGATAGCAGTGAGTACTCGTTCCCAAGATGGG
ACAGTTTCATCTAGCTGCATGGAGGGCAGGGCCACCATGTCACTTACTCCCCAGAGCTGAGGCCTGCAGACTGGGACCTAGTCACATAGAGCCT
CTAGTTTATTGTCTACAAAGTTAATACTGCAGTCTACGGTAAAGGGTCAATATCCAGTTAAGCGTCTTAAGAACATGTCTGCCAAGGTAACACG
TCTTCCTCAGAGAGTGCATTATATTCCTTACATGTAACACCAGTTTCTTACTATGAACACATGACTGTAAGCATAGATTGCTCCTGCTCTAAAA
AAAATTACCTTAATTTTTGCACCTGGCAGCACGGAAGAAAAATTGATAGAAGACAAGTATCCCTCTTGTCTCTCTTTATCCCCAGTTTGAAATA
CATTTATTAAGACAGGAACGTCAAGGTAACTAAATAAATGAATAGTATGTCAAAATCATCTTTCAGGCTGCAGATAATAGAATGTGCAGGGTTT
CCAGTAGCTTGTAGAATTTACTGTGTGGCTCTATTGTTCGATATGCATTGGGCTTAACAAACAGTTGTTTCAGGAACCCTGCAAGCCTCACTGA
ATGGTCATAGAATACGATGCACAAGGAAATCCCATGTTCTGCATCCAGCCACGCATGCGTCTGTCTGATTTAAACAAGTGTGAACGGAAGTGAA
AAGTTTATTGAATGGAAAAGAGTCTTACTGAACAGAGATGAGCTACAGCTGTTGGAGTTTTTAAAGGGGGAAGCTTTGCCCCACTCACCACTAT
GGGCAGCTTTGCCCCAAACCACACAGAATGCTAATTTAGCTTGGGGGAAAAAAAACCAGAACATCTTCACTTTTCTTTGAAGAATATGTGTGTG
TGTGTGTGTGTGTGTAAATGTGTGTGTATATATATATATATATATATATATATATATATATATATTAGCTTAAAATGTCATTTTCTTAAAATTT
TTAAGAATTTTACAAATGACATAAAGTTAGCATGAAACAAGCCATTGGTCCAGAAACATCAGACAGCTCTCTCCTTCCTCATCAGCCTGTGTGT
TAGAGAAGCCATATGCCACCATCCCTCCCTGACCTGCTCCTTTCTGGGGAACCATGAGTGCCCCAGACCCAAGAACTCCTCCTAGTACCTTGGA
AGTCTTTCTTGCTCTATTTGTGTCTTGTTAAAGGCAGTAAGAGTGAGCACCATCATGGAATCTTTGTGAGGCTAATGCTAAGCCAGGGTATGCA
TTCTCTTCTCCCTTTGAACACAAGCCCCGAATGCATCCAACCTGAAAATCGTGAGAATGGACAGAACAGCAGGATGTGTGACGGGAGGGGAGGA
GATTTACCTTCTCTGTCACAAGGTTCAGAAAGGTACGTACACACCTGGTCTCTAGATGTAGGGTGTGGCTGTCAGAGTTCTCTGGAAACAGCCG
ACCAGGGGTGTTCTCCTTTTGCTGTGGGAGTCTCAAAAAGACAGCGTTCTTCAACACCATTACACGCATTGCAATCACATGCTTGAATACGCCC
GTTTGCAGATGACATCCAGATTCGGTTTTATGAAGAGGAAGAAAATGGCGGAGTTTCGGAAGGATTTGGGCACTTTTCCCCCACGGATGTTCAT
AGACAGGTAGGTGGGTTATTATTGCGGGTTTTATTTTAATGCACTTCTGCCTTGGTCAAATTCAGTCCTCAGTTTCCACATATTAACAAAGAAA
CCGTCATAAGTCTCTTTGACATCCATACAGCAATGCTATGTTTATGGCTCTTCCAGTACTCTGACATAGAGGCTGAGAAGTCTAAAGGGCTGAA
GCTGATGCTGTCCTGATGAGCTGCTGTCTCTGCCTCATTCGTTACATGACCAGGCTCTCCCAGCTGATGACATGAGCCACCACAGTAATAGTCA
CTCTTTGACAGTGACTGAGAAATGGTGTTTCAAATAATTGGCAACAACCCAAATGTTAGCCAGTAAATGACTAATTAGATAGTGGTGTATCTAT
GTAATGGAACATCATACAGTTACTAGAAAAGAACGCTCTGTGAATGAACAAAGACCTCAAAATAGAATGCAAAGATTTGTAACAATCGATATGG
AACACACTGTATGTAGAAACAATTCTATGTAGAAACAATTCTGTGTTCACCTGCACACGAAGCCTGAGACAATAACAACATCAGCTCCCTCTAG
GATCTCAAGTTAGTGCATGAGTGGGAAGCGGGGAGGCAGGGAGAACTGTCAGCATATGCTACCTATTGCTCATCCTATATTTGATGACATCTTG
ATCCTTATAAACATAGAAAGACCGAAACTAAGTCTCTCCAGTTCCTTATTATCCCAGCACTGCCCAGGACTAGCATGTGACACCTCCTGCGCAG
CCTCACTTAGCATCACTGTCTGCGGGCTGTCAGGGGCAGCCCTGGTTCTGTCTGCTTCCACATCCACTCCTTCTCTTGCTGCTGGCAGTGATCC
TGGATGTTTTCAAGGCTAATCTGAAAGAGAAATGGCTGAGTCGCGTGATCCGCCATCATGCTGTTGTGTTTTACTTTCACGGGGGCAGTATAAG
TGTGGGTATAAGGTACTAACTGCCAACTTTCTCCAGGTGCTGGGTTTGCACACACTTTATTTAAGTTCAGAGTATACATAGACGTGTATAGAAT
GTGCCTGATGCTCACCGACACTAGAAAACAAATGTCCTACACTAATGTTTGGGTCTTTTGCACCCTGCAGTTTGCCATTGTCTTCAAAACGCCA
AAGTATAAGGATGTCAACATTACAAAGCCAGCTTCCGTGTTTGTTCAGCTTCGGAGGAAATCAGACCTGGAAACTAGTGAACCGAAACCCTTTC
TCTACTACCCTGAAATCAAAGGTACCTGCCTCCTTTAGACTCTTGTGCTTGCTCTGAAAGACCAGTGGGCCCCTCCTCTACTCAGCCTCCCAGA
AGCTGCTGCCTGGATGCCTGGATGCACGCTGCATAGTGTTAGGGGCAAGGAAGGTCATATGCCAAGGTCATGGAATTAGCCCCAGGAATTTATG
TTACTATATTGCTGCCAAACTTATATGACACTTACACATTTGGCTATTGTTGGGTATTAGAATCTAGTATGGAGCCAGCAAGATGGGTAAAAGC
ACTTGCTGTCAGGGCTGACCTGAGCCTGAGTCCCAAACCTGTGTGCTGAAAGGAGAGACGTACCTGCCAGAAGATGTGGGTGGTCCTCCACCAG
GAGGGGGCTGGGCGGGGAAGAAAGGGAAAGTAAAAAAAAAAAAAAAAAAAGTAAGGATTTTTTCAGCTACTCAGAAGCCCTTAAGAATTAACCA
GAAGGCTTAGCCACTCTCCAGCCCAGAACTATAAAATGAGATGGTCACTATACTTTCCTCACAAAAGACACCTCAGGAAAGGTGCACTATAGTT
AAAATGCAGATTACGATTCTTTTTGACGTGCTAGACATCATATTCTCTGGGCCTTGTAGACACTAGGCAGCTCCTCTACCATTGAGCTGTAACC
CTGGTTCTCAAATGATAAGTACCCAAAGGGAAGCATCCACAAACTGTATCAAATAGTTGAGGAGACAGTCCTGGATTCCTTGGTCTAAAGAATC
AGCCAAAGAATTTCTCTTCAGTGGGAAGCCACTTACCACATTATGAAGGTGAATGGAAGTCAAGGCTCTGTTTATGAAAGAGAGATGTTCAGAG
GCAGAGGTGTGGCAGCTGGTGTTTGTCCTGGGAGAAATGATTACTGTGAATCTGTGTGGACTGAGGAAGGTTGATTTAGGCGAGGGAGGCCAGG
TCCACGCAGTGAAGCCTTTCTCTGGTAGCTGGAGGTTTTTGCTTTGACTCTGATGCCTAGGCCTGCCTGCTCACTGTCTGGAATGATGTGGTCA
GAATGTTGCTACAATTCAGTGGTGCCTCAGCAGAGTTAGAAAGGGTTCTAGCAGCCAGAAGTTAGCATCCAGCACATACTTCTATGTTGGTGCT
GTGGAGACCTGGACCTTGGGTGAAGAGAAAGGAAGAGAGACTCGAGAGTCTCAGATTGTTCTAGGGTTTCCAGACTACAAGTGTATGTATGGAG
TGGTTAAGTGGATAAGAGGAACAAGAGAGAAGAGTCTGAGAATGAAAGAGAAGCTTAGTGTAAACATATTTCAGTTGGGGTCAAGTGCAGGAAG
GGGGCCAGTCAAAGCCATGACTCAAGTGGGTGCGGGCTCAGCACTGAAGAGAATCCCGGCCCCACAGAGTTTAAGTGTAGGGACGCACAGTGCT
GCCGTCAACCTCTCTGTACCTCTGTTTCGTAAGTTTTGTCTTCACCGCCATGGTTCTGAGAAGAGATCCATGAACTTGGAGGCTACACAAGGCC
TGGGATCTGATAAAATCAGAAATGTCTGGCTCAGAGAGAATGGGGCTGGAGAGGATGTCACCAGGTACAGGGTCCTAGGTCCAGCGCCACCAAG
GCCCCCAGGTATCCGTCCCTTGAAGGCAAAGCCTGATGGTGCCCAGGAGTTTCTGGGAGGCCATGTCTCTTTTTGAGGTATGGCCTCCCCATTC
CTGGGTCAGGCTTATGGTGAGCTCTGTGGCTGGCCTACTTCCTTCCTCACCAGCTTGAGGTCTTCTAGATCCTTCTGTCCAGACAAGGCAACAT
GAAATTACTGCTTTACCTGAAGCCTCTGCCCTCTGAAAACAGGAAAAAAACATTATTTTTCTAAGAAAAAAAAAAAAAAAAAAACAGTGTATCT
CCTGAAATGTGCCTTTAAAAGAGCGTTGTCATTGCTGATGCAGAATCATGAGAAAAAGAAAAGAACTGCAGGTTCCCTGTGCAGGATGTGAGCC
TTGAAACGCTGAGAGTTTGGTTGCCACGCAACCTCTCCTCCTAGTGTCTGCATCTGAAGAGTACAGTAGACAAGGCCCAGCTTCCTGGGCACTC
CACGCCAGCAGAGTGGGAGTTTTCTAATGCCTGGATTAGAGGATTGGGGTTCCCTGAAGTCCTGGTTACCTCACATCTTCTAGCTCATCATGAA
TACAAATGGTATACTTGAAATACAAATGAGGCTCCACCTACACACAGGATGGCTGAGGTACCTGTTGTGTTCCGTGTTTCTACACTGGAAACAT
TTTCTCTGTGAGATCCACAGTTCATCCACTGACTAAATGAATATAGCTATTGGTTTTGGTATAACTCCTGGGAAAACTTTATAAATATTTCTCC
GAGCCATCAGTTAGAAAGTAGACAGTCTCTTCACCTCAGCCTCACTCCTTTCTGGCCGAAAGCCGTTTTCTTCCTAATCATCCTGGTGTGCCCA
GGGTTGGTTTCCCAGGGACTGGCAAAGTCCACCATGCTCTCCTGCTTTCCCCATCAGCTGCAGTAGACAGTCCAACATAGTCCAAAAGTCCTTG
TGCCACACCGTGGGGTGATGGAAACACCCACAGAACTAGAGAAGTAGGGATCTTTCTGAACATAGACCATGATGAAAGCCAGCATTAATCAGTC
AGACACAGGACGATTGTTCAGTATTGGCCTATTTCCTGGATAGAAAGCACCATGTTGACTATTTAATGTAAACACAAAACCACCTTCACTTCTC
AGCCCCAAACACAAAGAAAACAAACAAACAAATATCTGCTAGGATACTCCCAGACCACAAAGGAACAGGAGCACCATGTCAGATCTCCCACACA
GTAACAGGACTGAGAACATGGTGGCTGAAGATCACACGGGCCATGACAGAACTCAGGGCACATGGCTGTCGATCACATTGCAGTAACAGAATTA
GGGGGGCTGTCTGGGTTAGTAAGGAACACAGACAGTGAAAGACAAGAGGAACACAGAACTCTGTGAAGAGCACCAAGATAAGTCTCGGACCACC
TGGACCTGGGCAGGCCACTGTCCCAGAGGTCACTCACTGTCCCAGAGGCCATCTTTATTTTGGAGTCCGAGATCTCACGAGCCAAAACATCTCT
GTACACCCTCTCTGTGTTTCTAGGATCGACATTATATCTCTTACCACAATAACCAGTAAGGACTCGGTCTCCATTGCCTCATGCCTGCACTGTC
TTCCTTAGAGTTTGACTCTAGATGTCCTGTATACAGTGATAGATGTAGCTCCTAAAATGCTGTCCTCTATGTCATTCTCTCAGACATTCAGCAA
GTCCCTCACATTCCTATCCCTGAGGCTCCTCAGCATGGGTGACCCCAGTCCTCCTTTAACCTTTATTATCAAAAGCACCCACCCATGTTATCCC
TCTGAGAAGCCCTGTGTCTTCCCAGAATATCATGTTCTTCGGCCATAGTTGTTAGCCTTTATAGAGGCTTGTCATGCTGTAAATCTGTTTTCAC
GTGATTTCACATCATCGTATGATGCATACCACCATAATCCATCCCACTAAATCCCTCTTTGCCTTGAAATTGACGGTCCTTGACACCAGAGATG
TGTGTTATGTTTCTGTCCATCCCAGCACCTTCCTCAGTCTATGTACCCAATAAATGTAAGCTGGTTATTATAGTTGTCTTAGGTGAACAAGGAC
AACTGTTCTGAACGTTAGCTGGGTGTGGTGGTGCACACCTCTAATCCTAGTGTTCACGAGGCAAACGCAGTTTTGAGTCTTGTTCAAAACCAAG
CTAGTCTACATAGTGAGTTCCAGGCAGCTACAGTTACATCGTGAGACCCTATCTCAAAATAAAGAAGTAAAAGTGCTTTAAAAAGAAAAAAATA
TGTGTATCTTTGGGTTATCTGAATCTTGAGAAGCAATGAGATCTCTCACCATCTTGTTACCACTGGCAACTTAGGAGGGGGCGCTCAGGAGAGT
TGGGTGTGTGCCGCACTGTGGTCTCTGAGGCGTGCTCTTGGTCTTCTCGTCCATTGGAATAAAAACCATGCTTCCTCCTCGGGTGCCTGTTATT
TCCCTTTAATGCATTTAAAAATATCATGAGAATCAATCGTCTCTCTGCGGCTGCTGGTGGAACCCGATCAGTCTTTCCCCCCTGGTTTCCCTTC
CTGTAAATACACACAAAGAGGAAGTGCAAAGGAAACGCCAGAAGCTTATGCCGAACTTCTCGGACAGCTTCGGCGGCGGCAGTGGAGCCGGAGC
CGGTGGTGGAGGCATGTTCGGTAGTGGCGGTGGCGGAGGGAGTACCGGAAGCCCTGGCCCAGGTACGAGAAACATCTCTTCTATCTAAAGCCTT
TCATGAAGAGGAGGGGGTAGTTTTCTTTCCCAGGTTGCCAAACATGTCTGCCCATGGAGTGATGGTGTCATTACTAATTATTAGATTTCAATTA
ATGGAAAATTTCCTACAGATGTAAAAGAATTTTTGTTCTCAAAGAAATGTATGTCCAACTCAAGGCACAGCCACTCCCCACCCACCCACCCATC
AAGTTTCACTTTCTCTCGTTTCAGTTACACGCGGTCAGCTTGAGTCTAAAAATATTACCATTGTGTTGACAGAGACAGACCACACACACAAAAC
TGTCTTCACAGTCTGCTGTTGTGACTGCCCTATTACTCTGTGTTCTTGGTCTCCTTCTGTCTTTTTACGTTGTGAACTTTACCCACGTCTGTAT
GCATCTGCAGTTTTAGGGATACACTGGGGGTCTCGGACGTTCTCTTTCAGGATAATATGTGTCTAGTCCCGAAAAGTGTAAGAAAAAAGTCAAC
TGAAGGGTTTCTGGAAGAAAACCTTTCTCAAATCATTTTTGACTGTTTCCCTCCTCCCCCTCCCCTTTGCTTTTGGAGAAGCAGTTAGTCTATT
ATTCTGAGAAAATATTAAAACAACTATCATACCCACCGCATGGTAGATGGGTATAATATGCCTACACAGCATACTCTGGGCTGGAGACACTGTA
CTGTAAACAAACTGAGTCACAGAAAATCATGCCAGGGAAGCACTTCAATGGTGTGTAAACTACTCCTCCTAACTGCCACGCATACGGCACAGTC
AGTATTTATGTGTTCCTCTCGGGTCCGGCTATGATCCCCGTGCGTGGATTTCCAGCCATCTAGTTACACTCAGTTTTTAAGCATCGTTGCATCA
TGCTTGTCGATGCTGAGTAACGCTGCTTTTGCCTCACATCTACCTACCTTAAGCATTTCAATGGAAATACAGTTCTTATAGCAGCATAAGTGTG
CTGTTTAATGACTCACACCTGTAGCGTAAGTGACTTGAATGCTGTTTCCTGTCTCCTCTGTAAAATAACTGTGCTGAGCATGGCCTTTCTTTAT
TCCAGATAACATGTAAATGCTTTAAATGTCAGAGAGTGTAGTGCTAAGTGCTCGCTGCCCCTCTAAATTTATCATTCATTTCTCCACTAAAGTA
TCATTATATTACGTATTGGGTTCTTCTCACCAGGGGTCTTCATATTTAACTATTTTTTTCTGTACCTCTAAGTCTTGGTGTGAGTACTTACCGT
TAGCTTTTCCCTTCACCTGGAAGAAAGGGACCTGCTCAGCCTTAAGTCCTCCAAGCTGCTAGTCTCGGCACTGCCTGTTTGTTCAGCTGTCTGT
TTCCCACAGTGATTCTGCTAACTCTCGGGCAGAGCCAGCCTGTGTAGGCTTGTGCCTCCCCATGCCACAGCCTGCCTCCCTGAGTGGGTCCCCA
TACTGTTAACAATCTCCCTGATGTTCCCTTTGTCTCATTGCTGGTTCAGCGTATGCCTACTCGAACTACGGATTTCCTCCCTACGGTGGGATTA
CATTCCATCCCGGAGTCACGAAATCCAACGCAGGGGTCACCCATGGTAAGTCGGATCGGAATACCCAGAGCACTGCTCGGGCAACAGGATCATC
TCTAAAAGCTTTGGAAAATTACTTTCCAATAAATTAATATTCTCAGGTTATTCTAGGACAGCATAAGGGACACCCCTGACACGTATTTGCTCAT
CACATTGTGTGGAAGCAGGACAGACGCTCTAGACAAAAGATGAGACTTGACTACGCAAACACACAAATACCACCTTCACATCTGTCCCTTTACC
CCAAGTCCTATCCTTCTGAAATCAGTTTTCATTTTGTAGGGTTGGCCTTTTCCCACTCTTGGGCATCCTCTGAGTAGCTGTGGCTACAGGCTGC
TCGTGGTTTATATAAAGCTGCATTCTTCTGTACCATGGAAATTATTCTATAAAGTCTTTCCATATATAATGCCAATTCATGCCAGATGCGTACA
TCACTGTAATCTTTTTTAAAGATTTTTAACTGGCATTGTCGTTTATCCTATAATCGGGTAACCATTCATCCCATAAAATAGAACTATTCTTTCC
ATTACCTATAACCTCATGACCCCTGTGCAAGGCCAGTGCTAGGCTTGGCACCTTCTGGACAGAGGTGAGGTTCAGAAACTTGACTTTCAGAAGA
CACTGTAGCAGGATTGGGTTTTGCTCCAGGTTTGCCCAGGAGTCTTACTGTCACTCTCTCACATAACGAGGTTCCTACAGCAACCACAGTCAGT
CTAGTGATGGTGCCCTAGAGCCCTCCTGGCCACACAGAATGGAAATGAGCTCTGCTCGGCTCCCTTTCCCTTGCCACTCAGGATTAGGAAAGAA
AATAACTCCTTTCGAGAACTTTCCAACTGGCTATCAGCGCACAGAACTTTATCTCATTTTTCTTCTTCACACATAAACAAAATGACATCCATGC
CTTGTTTCTGTTTGGTTTTGTTTCAGAGAGAGACAGAGACAGAGAATATCACTGTCTAACCTTGACCTTCTGGGACATTGTCGGTGGAGCTCAC
TACAGCTGGCTACAGAATGAGTTTTGGGAAGGCAGGCTTTTAAATTGTTTCTGAATATTTAAAGTATTTCCGAAACATCCCAGGTTTTTTTGGT
ACACACCTTTAATCCCAGCACTTTTTGTGAGTTTAAGGCCAACCAAAGCTACACAGCAAGACCCTGTCTCAAAAAGTAAATAGAGAAGGAAATA
TAAACTATGTCCAGAACATATTATTTGTGTTATAAACATTTATAGTTTTCCATTTCCTGCAGATAAGCCTTCTTCCTATTTTCCTTTTTTCTGC
CCTATGTTGAGACAGTGAGCTATACCTTAATAGATTTTCTACGTTTTTTTCCCTAGGCACCATAAACACCAAATTTAAAAATGGCCCTAAAGAT
TGTGCCAAGAGTGATGACGAGGACAGTCTGACTCTCCCTGAGAACCAAACTGAACGTCAACCGCCCACCCTCCCCATGGCCTGCACCAAGACCG
AACCCATCGCCTTGGCATCCACCATGGAAGACAAGGAGCAGGACATGGGATTTCAGGGTAGGTGAGCTTGCCCACGAGGGCTTCCAGGGACAGC
TGGGGTGGGGTTTCAGGGTAGGTGAGCAAGCCCACAAGGGCTTCCAGGGACAGCTGGGTAGGGGCAATTCTAGGTACAGGAAGCTGTCTCCAGA
AGACAGGTTTCCTTCCTGTTAATCTAAAGCTGATGAGTCAGCATCTCCTCTGAGTGCTCTGCACCGTGCCCTGTGCCCTGCAAGAGAGGCGTCC
TGTGGATCCAGTCATCTTTCCAGCCTCAGCTCTGATTATAAACTGCACAAACTTGCATGTACATTGCCTAGTCTGTGCAGTTCTGGGTATGAGA
AGAGGCAACTCACTTTGTCCCTGGCCCAGGTAACAAGTCTCCATGTCCTTCGCGCACCTCCCTCCCAGCAGCCGGCTCAGCTGCCACACTGAAG
ACGTTTGCCTGAAAATGGGAGTTTTCCTTTATTCTCTTGTCCTCCTTGAAGAAGTAGAATTGAAAGGGAACGTTCCATCTGAGCAGGCCTCATT
GGCATATGAGTCATGATTCTCGATCAGACGCCATCTGTAAAGAGATAGTAACTATTTACACATAGTGGCCGTACACATGGTTACTATCACATAT
TTTTTTCCCTTTTTTCTGTTTTTAAAATACTTCTTCTGAGATGCACAGTATATGTGTACAGAGCCATATGGAAACAGGGTAGATTTGCCCATTT
GGGTTGAGGTTAGTCTGCTGTCCCTTGACCTTAAGATAATAAAATTTCCAAAACCCCTGGGGTTTCTGAGACTGCCTGCCACCCAAGGGTGTGT
TTCCTCACTTTGACTCAGACAGGATTCCAGAAGAGACTTTGTTTGTAAAGTGATTGGCTATTTAAAATTATGGCAAAAGCCAAGTACCCTGAGA
TTGGTTGCTCCCCTTCAATCATAGACACTGATTGTTCTTCTCAAAATAGCAGATGTTTCTAGATTGTTCCTCACAGACACTTTAAAATCAACCT
CTTAACTGTATTATCTGTCAGTGAGACAATGCCCGAAATGCCCGATTGTGCTTGTTCTAGTCAACACAATCCAGAGGCAGCAAAAGGAGCATTT
TCAGCTAAAATCGTCTGCCATGCCGCACACGCGGGGCCACCAAATCACACTCAGCTGTGCTTGTGGGCATTACCTTGCAGATAACCTCTTTCTC
GAGAAGGCTCTGCAGCTCCCCAGGCGACACGCCAACGCCCTTTTCCACTACCCACTGACGGGGGATGTCAACATGTTGCTGGCCGTGCAACGCC
ATCTCACCGCCGTGCAGGATGAGAATGGGGACAGGTCAGTTGAGTGGGGACTGTTGCACAGCAAGCTCTGTTTCAGGACGGCGAAAGCCAAGGA
TACTGCCCCCACAAACTGCCTTTGAAAGGGAGCTGCCCTCATCTGACAAGACCTCGAGTATGCTGTCTCCCTAGGCAAGAAGGCTCAGATCTGT
GTAATCATGCTAATTTTACACCCCTGCTTTGTGTACTAAAATAGTAATTGAGAAAATTATTGACAGCCCTGCAAACTCCCTGAAGTGTTATTGT
CATAGTGTACCCAAGAAAGCCAGGTAAGAACAACATTAGTAGAGTAACTAAGAGGAAATGCCATTCTCTCCTAGCTACTAGCTGCTTCTCTGTG
GCCTCTGTTAAGGGTCACGAGGATGTGAGTGGGTCACGCTCTCCCTCAGCTCCAGGCCAGGCTGTGTCCGACTCTGACATTCCACATCCCATGC
CTGCTGTCTCTTCCTGAGGTTCTGTGCACATAGCTACCTGGCTCCTTCCTAAATGGTTCTCTTAGTCCTTCCATTGAGACTTCTGAAATGATCT
AAAAGCCTATAAATATAGCCAGAGACAGCAGTCTTGAGATTCAAGTGCTTTATCTAAGTGATCCTAAAATAGCGTTAACACTGAAAAGAAGCCC
TTTGCTGTGGAAGCATTCTGGAGTGCTGTGTGCTGAGTCTCAGTTTAAAAACAAAGCGCTTTAGGAGAATGCCAACCTCAGCGGTTTCGTACAA
GGACCTTTGTCCTCGTCACTTGCAGTGTAACTAAGGATGTGGTATAACCGCTGCTCTTATCCTACACAGTACAAGAGGAACCCAGACGACCTTC
TGTGAAAAAAGCTCTGAATAACACAACGCCCCTTTAGAAGGAAATCATCCAGTTAGCATTCAATAGCAAATAACCACAAAACACAGCAGATAAC
CAGAGCAGGACACCGGTGCTCCAAGGGTCATCTTTAAGGACTGGCGAAATTCATGAGTAGTGAGGGCGAGTGGATTAAGCAATTAGGAAGTGAG
ATGCTGCTGTCTGAAGGAGAGGCTGTGGAAAGGGATGCCAGGCACATGCAGTCCAGCAAGTGTGTGCAGAGGAATGCAGGAACATGTGTGTAAG
GACCTGGGTCTTCATGGGGCAGCTGAGACACTGGCCTTCATTCCCAACTGGGTACAATACCCAGAGCCTCGTCAAGCCAAGACTTAAACAAGCA
GGCTCCGTGTAAGGTTAGGGGTATGACACCTGGGAGACACAGAGGACCCACTCAGGAGTATACATAAGGGACTATTCTGGTTTCTCTTCTTTAT
TCATCTTGTCATATTTGGCTTATAATCTGGACCCTGTCACCATTAGTCATCATAGAAATACAATAAAGAGAAACTATAGAACCCTCTGGTATCG
GGGAACTTCCCCCAGCGGTATGAAGATCTGAAGGGGAGGGGACGTGCTTCTGTGAAGTGCTGAGCTGCACATGCAAATGTTGAAGTAGCTTTTG
AAGCCCTTTTACTCCACGTGTGTCTGTCCGCATCACCTCTCACAAGCCTCCTGTCAGGGTCCCATCAGGAGCACCACAGGAGCCTGAGGTGTGG
CTGCGGTGCTTCTCGTCAAAGCGTGCGTGCTTCACTTGCAGTTAACACACGCCTGGGGTCATCTAAAGACGCCACCATGATCTTCCCTGAGAAG
TATTGCTAAGTGAAATATCTCAGGGATAAATAACAAGACCTTGTTTAGAAATAGAAGTCACTTCTCATTGTTACGGACCAAGAGACCTCCGCAG
CTGAGTTTTCCCAAGCTGCTCCTAAGCCTAGGCATTACCTTTCTTCATGGTCTTGTATGTCAGGGAGAGACCAGGGAGAGCTGCCAGGGACAGA
GGCATGGACACAGAGCTGGCTTGGAGCTTAATGCCATTTGGTTTCCCACCCGCCAGCTGCCTTTGCACTTCATGTGTCTGGTCAGATGTTAACA
CTCCGGGGGCAGGTGGGCACGATCAGTGTGTAGTAGCCTGCGATAGGCAGTGGAGAGGAAGTTCTCCAGAGAAATAATGTCACCTCTGATCAGT
ACATTCAGTACATGGTAGGGCCAAGGAAAGCTACACATAGTAGCTAGTCTTCAGCTCCAGTCATTTCTTGACATGTTTTTGTTTCTGTCGCTTG
CTACATCCCCAGTTAATTATTGCCACTGTGTGTCATTCCAGTGTCTTACACTTAGCCATCATCCACCTCCACGCTCAGCTTGTGAGGGATCTGC
TGGAAGTCACATCTGGTTTGATCTCTGATGACATCATCAACATGAGAAATGACCTGTATCACCTAAGCAGCCACCACAAGGCCGTCGAGAAGAG
AAAAGGTTGCCTTAGTAACACGTGCGTGAGCTGTTCTCAGTGATGGGAAACAGGAAAACAGCATCTTCTCAGAAGTGGCATCTTAAAACCATCC
TTAGTTGGGAATGTCATTTTACAGAGAGATGCGTTGGGATCATGGGTTTGATGGCAATCACACTCCATGGAAATTCACCATCTTCTAGAAAAGG
CAGACTTTAAGACCTAGAAGTCAGAGTTGATAAAGTTTTGAGGGCAAACCTCAGAGGGGAAAAAGTCACAGCTGTCAGCAGAGACATGTTAAAA
AGAAATCCAAGTTTCAAAAGCCAGACTTACAGAGACAAAGTTTCGACCTCAGATGAAAGGATGGACCATCCAGAGACTGTCCCAGCCGGGGATC
CATCCCATAATCAGCCACAAAACGCAGACACTATTGCATATGCCTGCAACATTTTCCGCAAAGGACCCTGATATAGCTGTTTCTTGTGAGGCTA
TGCTGGGGCCTAGCAAACACACAAGTGGATGCTCACATTCAGCTATTGGATGGATCACAGGGCTCCAAATGGAGGAGCTAGAGAAAACACCCAA
GGAGCTGAAGGGGTCTGCAACCCTATAGGTGGAACAACAATATGAACTAACCAATACCCCCAGAGCTCATGTCTCTAGCTGCATATGTAGCAGA
AAATGGCATAGTCGACCATCACTGGAAAGAGAGGCCCCTTGGTCTTACAAACTTTATATGCCCCAGTACAGGGGAACGCCAGGGCCAAGAAGCA
GGAGTGGGTAGGTAGGCGAACAGGCCGGGAGGAGGGTATAGGGGACTTTCGGGATACCATTTGAAATCCAAATGAAGAAAATACCTAATAAAAA
ATTGGAAAAAAAGAATATTTTCAAATTAAAAAAAAAAAAAGAATTTTGAGGTCTGCGGAAGGAAGGGTATTCGCACAGCAACTGGATCAAGTGT
ATGTCAAAGAAAGGCTCTGAGGTCAGCTCCTGGAGAGGTGAAAGGCTGTAAGAACAGGAAGAGGCACCTCAGAGTCCCATCCAGCTCAGGTGTG
AGAACGTAAGCGAGCCCCGAAAACTCCAGAGGAAACCACCTGCAGATGGCACAGTGAGCTGAGGACAGAGGCAGATGGTAGGGGAAAGCAAACC
AGAAACTCCACTGGCAGACACAGTCTGGGAGCTGTGGCCTGGCCCAGGATGCTGCCCTGCTCTACGGCTCCTTCCTCTCTCACCTCTATTTCTC
TCCAACCCTTTTGTGCCCCTACCCCTCTCTCTGGGCCTGTTGCTCTTATCCTCTTGTTGGCTTCTAAGTTCCTCTGCACTCTCTTGCCCAGAGT
GTCATTCTGATCCCCACAGCCAGGCCCATGCTGAGACCTCTGCTTCCAGGTCATATTGCTTTCATTGAAAAACTGTGGCCAGCAACCGTGAATA
ATTTGTTATTTAGAAGAGATGAGATGACAACTAGTGGAATTCTTACTTGTTTGTTTGTTTGTTTGTGATGTTTTTTGTTGTTGTTGTTGGTTTT
TTTGGTTTGGTTTGGTGCTTGGGACTGTACCCAAAGCTGAGCACAAACCCTTCCAGCAATCTACAACCATGTTACTCCATTTTTAAAACAAGTT
ATTCAAGTTGTTATCTCTTCTTATCCATGCTTCTCTCTGTCTCCCTCTGTGTCTCTCTCTGTCTCTCTCTTGTCTGTCTGTCTGTCTCTGTGTG
TGTGTGTATCCCTCTCTATCAATCTCTCTCTGTCTCTCTGTCTCTCTCTTTCTCTCCCTGTGTCTGTGTATCTCTCTCTATCAATCTCTCTCTG
TCTCTCTCTCTCTCTCTTTCTCTCCCTGTGTCTGTGTATCTCTCTCTATCAATCTCTCTCTGTCATTCTGTCTCCGTCTCTCCCCCCCCCCCAA
TGTGTGTGTATCTCTCTCTGTTGATCTCTCTCTAGCGTACCCAGGCTCGCACTGAGCCATCAGTGTCCTACCTCAGCCTCCCAAATCATAGGCT
TGACAAGCATGTGCCTGGCATGTTATCTCATTTTTAATTTAAAAAAAAATCATTATTTTAGAAGAAATTAAAAAGAAACAAAGAGATGACTCAG
TGGACGAAGTATCTGCTGTACAAGTGTGAAGACCATGTTCAGATCCCCAGACCATCGCCAGCCAGATTTAGTACCACACCCCCCCACACCCCGC
CCCCGCCCCCCCCTCCCCAGTCAGGGCAGGAGACAAGCATCTTGCATATGCGGGGGGGGGGGGCGGACAAAAGACCCTGCCTTATAACCTTGCA
CAAGGCAGGCCGCAGTACACACGTTGCCCTCTGACCTCATGCACCTCCCTGCACCCACACGCTCAGAGAAGGAAAGAAGGGAAGGAAGAAAGGA
GGGAGGGAGGGAGGGAGGGAGGGAGGAAAGGAAGGAAACTAACTGCACATACACACTAAAACAAAAGAGCTAAAAAATTTTTATTTTAAAGGAA
ATCATTAAATTCTTATTTTTTAATACAATATTTTAAAGTTACTTTGAAAATATGAATTGGAGGCTGGAAGTGGCTCACATGCTTAAAGCCTTGA
GTTCGTTCGCTCCCCACACCAAAGAAAAGGCTGAAGCTGTTAGTTCGTTGGCCTGTAGCTCTCAGACTTTGCCGTTTTCAGAGGAGCACAGCTG
CTTTCTGCACAGCACTCTGCACTTCTCTGTCTGCAGTGATTCCTATCCAGGGGAGATGACTAGGCTAAGGTGTTATTTTAGATGTGTTCTTCAA
GATCTGTCTTCCTCGGGCTTCCATGCCATCTTGGGATACTATTGGTATTTGGAAAGTCATTGGGCCCCGAAAGTTGAATTCTCCAAACTTTTAG
GCAGATGGCACTTTTTAAAAACTTTGTATTGATTCTCTGTGAGTTTTGCATCCTTTTTTTTTTTTTTTTTTTTTTTTTTTTTGGTTTTTCGAGA
CAGGGTTTCTCTGTGTAGCCCTGGCTGTCCTGGCACTCACTTTGTAGACTAGGCATCATGCACCCCAGTCCCACTCATCTCCCTGTCCCCTCAT
ATCTGCCCTCTGCCCTTGCAGCCTCCCTCCCACAAAATAAACACTCAAGTAACAGGAAAGACAAAAACAAAAAAAGCATAGAGAACATCTCGAT
GTGGGAGCGATAGTGTGTCACCGGGCATCCCACAGTGTATCCCTCTGTCCACACATCTTCACTTGCAATGAGTGTTTGGTTCTCTGTGACACGG
TAATTCTGAGTTCCCCAAAGTATAAGCTTTTATGTTATTTCAAGATTATGCTTATACTCAATTTACAATAATAACAAACATGTGGCAAAAGTGA
GAGCCTGAGAACTAACGACATAAAAGGCCGTGTCACATGCCGGAGAATGCAAGAACAGAACGCAGTTTAAAGACTCACACTTGGCCTAGTCTAG
AGAGTGGGTGGCTTCCAACAGGGATCTGATAGTGACACATGGTCACAGAAATGTACTATGGTTAGTCGAAGATCTTTGGGGAGAATGTTTGAGA
TAATTTACAAATAATCCAAATTACTTAGAGAACTCTATACTTTTCTGCCAAGAATAGTTATTATGTCATGCATCATCGTGCATATCTATAATTC
CAGCCCAGAGGAGTCTGAGGCAGGAGGATTGTTGTGAGTTTTGGGCCATTCTGGCCTATACAGTGGGTTCCAATTTTGTCTGTATTATATTATA
GGGTCTTGTTTAAACAACAAGAACAACAACAACACAAAACCAAACAAGCAAAGAAAAAAAACCCTCCAAATTAAAAAGAAACTAGTTCCCAAGC
TGGGTGTGGGGCACACATTTGTAGTTCTAACACCCAAGAAGACACAGGACGACTGACAGCTTCATGTCAATCTGGGCTACACAGCAAGTTCTGG
GCCAGCCTCAAACACAGGAAGACCCTCTCTCAAACAAAACAAAGCAAAACAACTCCACAAAGCCAAACAAAGAAACCCAAAAGTTATGAGAATC
ACAAACGCTGCCTGTCTATGTCCTTGAGGCTAAGAAGCTCCTATGGCAGGGAATGTTTGTGCCCATTAACTGACTGAAGCCAAGGTGGGAAAAA
CTGGGGAGAAAGGTCTGAGGGCTCCAAATCAGAGCAGCTTTGCCTTCACCTCAGGGGAGAGGTTCCATCCACAAACAGCTGGGTCGCACACAAT
AGAGCCTGCTTCCGCAGATCCTATCACCAACAGCCAGCAGGGCTTCCTGAATCCAACCTCCAGCAGCCGAGGGAAGCAGGTTTTGCAAAGAGGA
TTGGCAAGTAGCAACTTCCTCTTCTTCAGTTTATTCTTATACTTGTGATGGTTAAAAATGCAATCTGCCACTTGTCTGCTATGAGGTAGCCTGG
GTGCAGGCCTCATGCCTTCCTCCACCTCGCCTCTCACTACCTCTACAGGCAGTCAGGAGAGCTGGCCTGGGCTCATGCAAGGGGGTGAGCTAGC
CCTGTCCTTCACAGGCTTTCGAGAGCAGGCCTTCCATCTAGCCTGTGCTGCAGAGTAGAGAGGACCCTCGTCGAGGGAGCACAGTGAGCAGCCC
TGAGGGTGTGAGAGCAGGAGAACTGGCCCTGCCCCTCACTCACAGGCTGTAGTGTATCGGACAGTGGGTCCTGCCCTTTGGGCAGCACAGTGGA
GCCAGCTCTGCAGGCATAGGGAGATCCGACAGCTCCCTATTTCCAGATGTGACTGTTGCATGTATACAGGAAGAGTGCTCTTCCAGTGTTGGCT
ACAGACAGCTGCTCTGGGAAGACATGTATTCTTTAGTGTCCTCACATGCACTCCACACGGTTTTATACATACATTTCCTATCCCACATCAGCCT
TTCCCTTTAAGTTTCAATTTAAAATACTTTCTCAATTCATAATTTTTGCAAAATTCAACTGAGAGGACCTGTATATTTCCTGAGATTGAACCAG
ACGCTTACCTCCTAAGTTCCCCCAGCAGTTACATCCTACTTAACACAATAATACAATTACCAATTGCAACAGATTCTAGTCAGATCTCAATATT
TGTATGTACTCATATGCATGGATAGCTCTGTAACATTTTTTTTTCCTAAGCTGAACATGGTGGTATTGTGTTTTAGTTCCTGTTATTCAGGTAG
CTAAGGCAGGAAGAGTCCTTGAATATAGATGTTCCGATGTAGCCTGGCCAGAAAGCAAGACTGTTTCTAAGCAGAGTGAATGAATGAATGAATG
AATGGTCAATCCATGTGCTTGTGGATGTGTATGTAGCATTGTACTGTGTCCCTGTCTTGGGTCTTCACGCTGTTTATCCTTTAGTTTCATAGAC
AGCTGGTTTGTTCTCATTGCTCCTGAAGACAATGCTGTCATGTCCCCTAACTAGCTGCTCACTGTCCACACGCTGTGCACTGCAGTGAGCACCT
GCACTGTACCCATGGGTGCCACAAGTGGGTTTCAAGGGCAGTGTGAGCCCTGAGCTCCACAGATGGATTCTAGGGATTCCTTGAGGCACGCATT
TTATGCGTGTGTAAAATGTAACCCTTGGGTGTTGGTGGCCCACCAGCCACTGCTTCCCAGCAGGAAGAACTCCCTGAATGGGAGGTCGGGCAAG
TCCTCACTACACAGCCTGGGCTTCAGTGTAAACCGCCTACAACACAGACTGGCTGGCATTGAGAGTTGCTGCTACTCTCTACCAGTGATCTCAG
CTTTTTGACAGACACTTTTGTGATTGGGTTAGCCCCACTTGTTTCTGAAAACAGTTTTCCATGCGTCCTACTTACAACCATGGAAGCACAGCTT
TGAGGTCAGGGTAGTCAGAAAGACCTCCCAGAGCCTATTATGCACAGTGCCTGGGATTCTTTTCTCCCTCAACTTTCTTAGGGAGAGTGCTGTA
TTGGCTAACTATGCCCTGAGGATCTGGTAGCACCTGTGATAGTATTACAGTGCAGCTTGTATCTGTGCGTCTCTTGAAGCCAGCCTGCAGCAGT
GTGTTCTGGTTTAGCCTATGTACTTCCACACCATTCTGGAAGGATGACATCTTTCTTGATTGCTTTCTTTTAATACTCTATTTATCTTTTGTTG
TTAAAAACACAGCATTGGTTCGATTTGCAAATGTATAGAATTACCAGCTATAGATAGATACCTATCAGAAATTCAGAGAAATTAAATTTTCTTC
TACAAGAGTTAAAAAAAATCATGTACCAAACGTATCCCATAACCCCGACTGTTGAATGCTGGTTCCCTCCGTGGGCCTTTGAGCCTGGCATGCA
GAACACGAAGCTCCTTGAGTGACAAATGTCCCTACAGTATGACTCCATAACGTAGCCTGAAGCCAGGGGCTACAGACATCCCTAAGCAATGGCT
CTCCCTCTCCTTCTCTGTGTACACACCTCTGCACTTGGCCGTGATCACCAAGCAGGAACATCTACTAGAGGATTTGCTGAGGGTTGGGGCTGAC
CTGAGCCTTCTGGACCGCTGGGGCAACTCTGTCCTGCACCTAGCTCCCAAAGAAGGACACGACAGAATCCTCAGCATCCTGCTCAAGAGCAGAA
AAGCAGCGCCCCTTATCGACCACCCCAATGGGGAAGGTAAGAGCAGTCGCCTTGCTGGCTAACCCTCCCCTCTCGGCTGCAGCATAAGGGCTTG
GCATTTGACAAATGCGTCTGACATGACTTTCACCATGAATACTCTCCTCGGCCAGCTGGAGTTTGTCATATCTTGTGGTTATTTCTGCTGTTTA
TTACAGCAAACGCCCTTAAAGAGTATTTGTGAGTATTGCTTAGTAAGTGGAAAAGCAAGCCCGAGCAAACATTAGTCTTTATAAAGCATCGTGT
GTGTGTGTGTGTGTGTGTGTGTGTGTGTGTGTGTGTGTGGAGAACGTTTACATAGTAGCTCAGTGTCTCCCCAGAACACAGTGGGAGCTTGTGG
CTTCTGAAAGTAGAACAGTGGACCGTCTGAGGCTTTGAAGCACGCCCTGAACAGAAACCGGAAGTGCTGCGACCTGGTGGTGTCCCTCCTCCCC
GCAGCCCAGCTCTGTCAGTCTCACCCACATCTCTGCCTTTGTTCGGCCTTAGGTCTAAATCCCATCCACATAGCTGTGATGAGCAATAGCCTGC
CATGTCTGCTGCTGCTGGTGCCTGCCGGGGCAGAAGTCAATGCTCAGGAGCAGAAGTCTGGGCGCACAGCGCTGCACCTGGCCGTGGAGTACGA
CAACATCTCCTTGGCTCGCTGCCTGCTTCTGGAGGTAAAGGCGTACTTGTGATCTTGACCTAAATCCCCCTGGAAGTTTTAGGGAAGTCTTTTC
AAAGAATGACTCTAGGGCCAGCTCGCTGTCAGTATTCCACTGTCTGCTTTCTGCATCCCCAGCTCCACCCCAGCTCTCAACCTGGGGATGCCAG
TGACAGAGCCAGCCCAGTATAGCAGTTGGGTGAATGATGTAACCTGAGGTGCAACGGAGATGTGGAGGTAGACAGTGCAGGAGACAGCCCCTGC
TAACTCTTTCTTTGTGGGCGGATTTAGGGTGATGCCCACCTCCACAGTACCACCTATGATGGGACTACACCTCTGCATATACCCGCCCGAAGAG
GGTCCACCAGACTGGCAGCTCTTCTCAAAGCAGCAGGTAAGACGCTGTGGTCACTGATACCAGCAAAGCAAACTGCCTGCAAGTTCAAAGCTGC
CTGCGGAAGCTGCGGTTCTGCCCTGGGAAAGTGCCGGTAAAGGAGACCTGCACCCGGGCCACTCTGCAGCGGCTGCACTGGAGGCTCTGCTTGG
CCTCGCGTTTACCTTGATGCTCACGCTAGTTCCCACTCTCTGGTTATCTACGTTGCCTTTGGGTTGTTGAGGTTTACTCGGTACCTATAAAATC
TTGAGCTGTGTCAGCTTCTGGTCATATTACAAGTCTCCCAGAAGGAATATCTAAAGACTTCCTTTTGCAAAAAAAAAAAAAAAAAAAAAGTCTC
TTAGAAGCTGAAAGCATGGGAAAAGCAAATGAGCCCTGTATTTGTGTTACTGTGCACGAGGCACTAAGACTTGCTGGCCTCCACTGCCTTTCCT
CGGCTCCAGGCACCATGCTCGCATCCCATATTCACAGTCTCCTTTACCACATCTACTCCTCAGTAACTCTCTGAGATAACAACTGTTGCTATTA
ATCCCATCTGAGCACCAAGAAGGCAGACTACAAAGATGATCAAGCCCCACTCCCAGTGTCCCGGAAAGCTGAGTCACAGCACCGCAGCCTGACT
GCAGACTCTTATTCTTCACGCTGTCTTTCTTTCTGCTCTGCCTGGGGTTCTAGTCCTTCCTGTAAACCACTCAGAAACATTCCAGCTCATCTCC
TGTCTCCGGCGCTTTCTGTGTTCAGTGTTAACACATGCTTGATGAGGTTGGCTGGCCTTCCGGGCCTGCCCAGACACACCTGATTTACTTCAGA
GGTTGATAAGGGAGGATTTGTGAACGACACATTCCTTATAGATTCAGGCAAAGAGGCCTTGGTTGTAATGTGTTGAGTCATTTTGGAAGTCACT
GATTATACTGGGGGAAAAAAAAAAGGTTGTCTCTTAGTCAGACACCTGAAATCTCAACATTCAGAAGGCTGAGGCAGGAGAATGGCCACAAGCT
TAGGCTACCTTGGTCCACATAGTTCTAGGCAGAACAAAGATACATAGTGAGATACTGTCTCGACAGTAAATACATAAATGTAAACACTTTTACT
TGAATTCACCATGAAAAGGTAGCAAATAATATCTTGTGTGACATAAAGAAAACCACACAAAAAGCCTAGCCCTGACCTCCTCGGAGTCACAAAG
GTCACTTCAGGGACATGTGTAGCCCTCGGGTCTTCAAACTCATTTCCCAGTCATGTTCCTTCCCATGTTTTGCTCACGTTATAAAAGAGCAAGT
GAGGCAGCAGAGAGTGGCAAGGCAGCAGTGTTGGCCCAAGTCAGAGGTTCTGTCGGCAGAACTGAAGCAAACACTTCATTCCAAAAGGCTGTGC
TGAGCCTGCCTGCCTGCCTGCCACTGTTCGCCTACCCGGAGCCGGACACCCATAGGTAGAATAGCCAGGAAGGCATCACAGGTAACTCCACTAA
CATTAGGCTTACGTTTTATGTTGTCTTGCTCGGAGCTGAAGAACAGGCAATATCCCCAAGATTTGTTAGCACATTTTTTCCCCATAACAACACA
TCCTGCCTAGCATAGCGGCCTTGTGGTGTCCATTTCTCTGTTGCTTAGGAACTAGAACTAGGGCCCCAACAGTACAGCACTCAAGGACAGAGCT
GACCAAGCAAATCTGCTCCACACAAGTAAGCGAGAGTGTCAGAAGTGTGTATTGGTAGATGTAAATGTGAAGGTTCAGGACTTTACCCCTGTGG
GTGCATAAATTGCCACCTACTAGTCCTTAGGTTAAAATGTATCCTTTGTAGTATAATAATAATAAGAATAATAATCCTGCAGAGTTACAAATCC
CAGGAGTACTTGAAGTCTTTCATGTATTTACTTTACATCCTGATCACGTCCTCCCCTCCTCCCAGTGCCCTCCACATGGCTCCTCCTCCCCACC
CCCTCCCTCTCTCTTCACAGGGTCCTTGGCTACCTACTTCCATAGAAGTGAATTAAAATTTTTTAAAAAGTTGTTTTAAATTTCACTAGAAATG
AAAAGTTATCCTGTAATTGCCTTAATGAGCTAGAGGTAACCTGTCCCTCGACATGGGAATCCTGTGTCTGATTGGAGGCGGACAAGCTTTTTCT
TTTACACAACACTGTATTTCCCTCACTCCATAGGAGCAGACCCCCTGGTGGAGAACTTTGAGCCTCTCTATGACCTGCACGACTCTTGGGAGAA
GGCTGGAGAAGATCAGCCAGTGGTGCCAGGTACCACACCCCTGGACATGGCTGCCAACTGGCAGGTGAGCTCTGGCCAGCCCATCTCATGGGTA
TTTACGGGGGAGCCAGTGGCTCAGGAATATAAAACAGATGGTCTTCTTGGCATGCTCCTCCCTAAAGAACATGCCTTTGTTTTTATCAGGTCTT
GTTACCGAGAGGGTCTTAATCTTCAAAGATGGTCTTCATCCATGAAACATCAACGTCCGGTGAGCCAGAGTGTAAAGCTGTTTGCGTAACTGGA
TTAGAGTTCAGATATGAGAGGTTAGAAAAGCACAAAATGCACAGAAGAGTTACCCTGTCAGCACACGACCAGAGTTCTTGCCATTTCTGTCAGA
AGCTAGCTTGCTCAGAGCTTAAGTAAGAGGTTTCTGAACATAAAAATAAACTAGAAATTCTCTTGTCCATTTCCATAAACATATTAAAAACCTT
CCTTAAAACACAATAGGGCTTACACTTAAAGGAGAAAACGCTAGGTGTTTGGACCAAGAGAAAAGACCCAGAACCCAAAGTAGGCAGTGTACGG
GTCCAACACAGTCGGGTTTGTAGGAATCTAGACTCTCTGTGCACGTCTAGGAGGCTGTTTGCTGGCTGGGTTCCTACACGGAACACAGAAGACT
GACCCCTGACCTGATTCCTACCCTGTGTACCATCAGCAAATTCACCTCAGAGAACACCAGGACTTTTAAAAATCATTTCTGTGAATATTTTCTC
ATGTTTTTCTATCCGACACCTATTTGACATACTAAATGGGAAACCGTATGAGCCTGTGTTCACATCTGATCATATACTACCACAAGGTGAGGTG
TGGCAGGACAGATTCTCTCAGCCAGTGTTGCCCCATGCTGTGGTTTACAGGAGGAGAGGAGAGGCCAGCCTGGGCGAGGGTGTACTGCAAACCG
TCTAGATCTTATCTCAGAATCTGAGATCCCTGGACGTGTGGCCCTGAGCTGCTAGAAGCACCTACTGCTGCTCCTGAGCAGGGCCTCTCCACTT
GGCAGAAGCTTGGTGGCCTCCATCAGAAGTTGCACAATGACCTGTCCTCTCAGATTGATTGTCCCTGGAAGGTTAAAGCAGTCATGTTTCTTCC
TTAGGGGACATGAAGCAGCTGACACAAGACACGAGGCTACAACTCTGCAAACTGCTGGAAATTCCTGATCCAGACAAAAACTGGGCCACTCTGG
CACAGAAGTTGGGTCTGGGGATATTGAACAATGCCTTCCGGCTGAGTCCTGCTCCTTCTAAAACTCTCATGCACAACTATGAGGTAACGCGCCG
CCGTACCATTTAGAGTTGTCTGTGTAACTATTGGTAGTTGACCGTGTCAGCTCCACTGAGCAGCACAGCCTACCCCCTTACATAGTAACTTTAT
GGAAGGAAACCAGTCATTGCCTGGAAGGCAAAGGGAGCCAGGCCAGGATGGTGCCTTTTTCTTGAGAAGACTGGTTACCCGTCTCTCGGCATTC
CCTGTTTTCAAATTCTCAGAGCCTTCCTGTCAAAATACTACTGCCCACAGGATGGGGACCCCTCCAAGCTGTGATCGGTGTGTGCATCCCTGTT
TGCAAGAGGGAGCCCCCCAGGGCCATGGCTTCATTGTTCTGTCGGGTGACTCCTATTAGAGCAATTCCAAACTAAAAAATAGACAAACATGGGC
GAGCTACTCCGAATGACCCTGTCATTGCTAGCTCCTCTCTTCCCTGGATTTCTGTGCGTAGACATCACCGTAGGCAGTCTCCTGTCTCCTGTCT
CCATGAACTCACAGGCTTCTTTCCCTTCCTTCCCCTCTTTTTTCTTATCTCTCTCCCCNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNCTACACAGACGCCATTGAAGTGATCCAGGCAGCCTTCCGCACCCCGGCAACCACAGCCTCCAGCCCCGTGACC
ACTGCTCAGCTCCACTGTCTGCCTCTCTCGTCTTCCTCCACGACCCAGCACATAGGTAAAGACCCTGAGAGAGTTCCTTTCCTCGCTGCCAAGG
TCTTTGTGCTCTGGGACGTGGCACTACACTCCCTTTCCCTAGTCTTTTTCCTGACATGTGTTCCTCTGTGGCATGCCACCCGCTCCCTCCCCAC
AGTCCTCCTCTGCGCACACAGGAACAGGCAGTTCCCACAGACACTCACATTGGCATATAGGTCACTTAGAATCCCAGTACACACTGGACATGGC
GGGGAGGGTTAAAGAAACACTACTTCTCATATTTGTTAAATTTGGAGAAAGATTCTAAGAAAGGTGCACAGCCCTGGGCCACAGGACAAGAAGT
AGGACCCAGGAAATATGCATACTGTATGACCCTTCCTTTCATGCCACCACCCTTCAGGTCCCCCACCTCCTCCCTCCCTCGGCACTCCCCGTCC
TTCAGCTCCCACCCTGCTCATGGTCCTGCCTTTGTCTTCCCACACTAACATCCACCTGTCACCCCCAACACATGCATTTCTACGAATGGATCTC
GGTACAGGCTTGAATTATGTCATTGTTCCCGGCCCGTCCTTTCTCTAACCTCTGCACCCTTTCTTCTTGTTCATTTTCTACCCTAGCCACAAAC
TCCACTGAGACCAGGAACAACTAGCATTCTCCCATCTTTGTATTATGCCAGCACCCCGGTGGCAGGCCTTCCATCTGGGCTCAGTAGGTTATGT
CTGCCTTCCACCTGGGGCAGTCCTCCCCTGGGAGCCTGCCTGTCACCTTCAGCAGGTCCTGGACACCTGATTCTTGCCTCTTTGTCCTGTACTG
TCCTTCCTTCCCCACCTGCTGTGGAGAACCTCTTGCTAGGATGTAACTTCTCATCTTTAAACACTGGCCCGCCTGGCCACAGGCTGCTTAGCCC
CGCTTTGATTAATGTTACATCTCAATGAATGGCTTTGTCCATTGATAGCGCTTTGGTTACCTCATGTTTTCACGTTTCTATCATACATGCTGTT
ATTTTTGTTCGCATTTCTCATATGGGGGGAATCTACAGGTAAACACAGCCTCCAAGTTCAGTAGAAAAATGTCATCATAAGAGTATTATATCCA
ATAAACAGTAAACAGATTTTTTAAAAGAGAAAGAAATTTAGAGAAAAACGAGACATCACTTCCTTGTTACACCCTTTAGTTGAGTTCAAACTTC
TCAGAAGAATATGATAACTGCTGGTCACCTCACCACCTCTGTCTCCCATCTCCTTCTGTCCCCTCCTCTCCTANNNNNNNNNNNNNNNNNNNNT
CCCATTTTCACATGTGTTCCCTACCCTTTCCATCCCTTTAAAGCCTGTTTTCCCCCCCCTGGAGGCCTTTGCTAGTTTTCTGGCCTCTGCTCAC
GCTCACTTCCACATGAATATACATACGTGACTGTTGGAAGCTAGGATCCACACGTGAGAGAGAACGAATGGTATTTATGTCTCTGGGCTGCCTT
GATTAGTATTCTCCATTAACCTGCAAGTTTCATCTCTCTTTACGGCCGAATAAACTTTTATCATCTTCACACAACTCTTGGCTCTTGCACAAAC
TCATACTGGCCATGACTGTCAAAAGTATAGCCAGGCTGTAACAAAATCTAAGAGTGGCAATGAATACTTTCTGGCATTCAGGTCCTCATGCTTA
ACCCTGAAAGGGCATCTCTTCTTGGCAGATGCCAAGTTCCGCCCATCCTCACTGCTTGCCTGAACTCCAACAGTGGCTCCTACTCAGCCTCTTT
GACTCTTTCCTTGCCCCTCCATCAGTTCTCTGCACCACAGTGATGTCACATGATAGGGCTCCAGTCACAATTAAAAACTAAATACTTAGCAGCT
AGGAACACGGCAAGTGTCCCCTCCTCTACCTTTGGGAAAGCCAGGTAGGGTGACAAATATGTACAACCCGATAGCCCACTCATAGAGGAAAGGA
CACCGAGAGAGGTGGATCCACGGGACTCACTGGCCAGTCTCTAAAAACAAGATGGAGAAGGAACAAGGAAGTCACACAATGTTGACCTCCCCAC
ACCCCACACCTCCACATGAGCACACATCCCACAAACACAAAGCAAATACTTAAATACTGTATTAAATTTCCATCATTGTGACCAACTTAGGAGA
GATTTGGTTGGGCACACAGTTTTTATAGGTCCCATGGTCCACTGCCTTCGCGCTATAGCAAAACCCTTCATCACGGAGGAAGCACATGGTAGAG
GAATCCTACTCACTTCAAGGTGTTTGGAAGTACACAGAGATGGGGAGGAGCCCCAGTACAGATCTCTTTCAAAGCCACGCCCTCCATGACCTAA
CTTCCTTCTGTGAGGCCCCATAGTCAAAGAGTTGTATCACCTCCAGATAATGTCACAGGCTAGAAACCAAGCCTTTACTTAGCACACAGCCTTT
GTGGGACATTTTAAGATCCGAACCATAATACACAGTAAGCCTTTCAAATGCTGCCCAGTCCCTGTTACACAACAGACAGTTGACTGGATGAGGT
AACACCAGGACACCGCTCTGGCATGGTCATTGAGTCTCAGTGTTCTGACGTTAAGATTCAATGATGAGAGTCAAACATTTGCTTAGTAAGTGTT
ATGCTTCTGTTTCTTTAGTCACTCTGTACAGAAATAATCTGCTCTGGTTCCCAGGAATGCTTTTCAGGTGATAGAATTTAACTTAAGCTTTTGG
TATAGACACATTTAAAAAAAAAAAAAAAAAGCCTCATTTTCTTGCACCAAAAGAAAATTTTCTAGGCAATGTGTATTCTTTAAAAGCTCATACA
GCTCAGAGAAAGGTGCCATTTTTAGTTCCTGCTTCTGTTATGAGTTTATTTTGCCTTGTATTCAGCAGTTTGCCCACCCGTTAAAAAAAAAAAA
AAAAAAAAAAAAGACAAAAGGTAAAATGATAGAGCAGTTAGAAAAATATGCCTCACTTTACAAAGGAAAACATGTTTATCCTGAACCAAGAGAA
TTCAGAACAGATTCTGTTCAACAGAAAGACTGATGTACCCTGTAGCACTCTCTAATGTCATTTGATAACCCTGCCATTCACACAGCCAAGCAGC
ACCGGTGGCATGATGGGGAGGTGGGATGCCGTGCTCCGCAGGAGCTAGCAAGCACAACCCTCCATACCATAGTCCACACCGAGCCTGGGAGGGA
GCCCACTGCTGTGGATGGTGCTCTAGGGCACTGTGACAAAGCTCTCTGGGTTTATGTCAGAAGGGATTCAGGGCAGGAACCCAAAGACTTTGAA
TCTGAAACTGCTGAGCACCCCGGCTTGCTGCCTCATGGGCTCATGCTCCGCTACCTTTCTTTTCCTTTTTTTGTTTTGTTTTTTTTTTTTTTTT
TTTCGAGACAGGGTTTCTCTACATAGCCCTGGCTGTCCTGGAGCTCACTTTGTAGACCAGGCTGGCCTCGAACTCAGAAATCCGCCTGCCTCTG
CCTCCCGAGTGCTGGGATTAAACGCGTGCGCCACCATGCCCGTCTTCCGCTAGCTTTCTTACACAGCCCTGGACTGCCTCTCAGGGAGTGGTGC
TGTCTACACAGGATAGGCCCTCCTCCCGATCAGTTAACTGTCAGGACACCCCCACAGACCAGTCTCATCTGGCTGGGCCCTCCCTCATCTGCAC
CTCCCTCCTCAAGTGATTCTAGACGTGTGAAGTTGACAGTTGAAGGTGACAGGGACAGCAGGCAGAACAAACCCCTTGCCATGGCCTAACTCAG
CCCCTCATGAGTCTGTGCTTTCTCCGCAGATGAACTCCGGGATAGTGACAGCCTCTGTGACAGTGGTGTGGAGACATCCTTCCGCAAACTCAGC
TTTACAGAGTCTCTTACTGGAGACAGCCCACTGCTATCTCTGAACAAAATGCCCCACGGTTATGGGCAGGAAGGACCTATTGAAGGCAAAATTT
AGCCTGCTGGCCGTTCCCCCACACTGTGTAAACCAAAGCCCTGACAGTCCATTGCATCGTCCCAAAGGAGGAAGGCAAAGCGAATCCAAAGGTG
CTGGAGAATCGCCGGCCTGCAGGGTCACTCGATTTCATTCAAGGCCTTCCGAATTTGGCGTCCTTTCTTGGTTCTGAAATGAAATGTAGTTGCC
ACGCACAGACGGTGTCTAGCAATCATGGCGCTCGCTCGCTCAGCTGCACTCTATGGCTCAGGTGCAGTGTCTTGAGCTTTCTCTGCTGCTACTG
GATCACATTTGCTTTGTGTTCTTACTGCTGTCCCTCCGCTGGGTTCCTGCTCTCATTAAAAGGTGTCGCTGTCCCCACCCGGTGTCCTTTCTAG
CCATCTACTGTAAGTTGTGCATTCAAATTAAGATTAAGGAAAAACATATTTTTAAATGAGTACCTTGATGCGCAATAAAAAAAAGACATTTCTT
TTTTTAATGTGGTTTATCTGTGATTTAAAAATAAAAAACACATGAACTTACAATATTTAAAACATGCTACAATCAGTGCTGAAAATAGTATTTT
CCCCGTTTTATGCATTTTACTATTGTAAATATGTTTTCTAAATCAAATACTTTAAAAGAAGAAATGTTGAATTTATAAATGCTATTTACTTTTT
TATTTTACTTTTATAATAAAAGTACAAGCACATTGTTGACCTAATCTTGTCTGATCTCTGCAAATGCTTTCTCAGAGGAGTCCCTGTGACTGTG
GGAACAGCCCCACAGCCCGTTTGTCACATCCACATTCTCCTTTAGACACATTTCTTTGAAGCTAGAAATGTTCAAACTGTGGAAATAAAAGTGT
TCTAACCCAAGACTGAGGTTCTGACCCAGGCAACATCATCTGGACGCCTAGCATCAACATTCCAATCTCTACACCGTTCCTCTCCAGCGCATGC
TGTCTGTGTCTATGAAGGATAACAGACCCCAGCTTCAGCACTTCATCTTCTGGATACGATGTGGCCGTTGCGTTCATAGCTAACTACACAAAAC
CTGTACAAGATGTGGTCCTTCATCATGTATAGAGAGGGACTGATGAAATACACACACACACACACACACACACACAGACACCACACCAACTGGC
TGTTAATAGTTGCTAGGGGAATGGGAGTCCTTTCTTCAAATGGTATAGCACATACTCCAGTAACTATTCCCTCCCCATGTTCATGCAGGCAGCC
CTAATTAAACTCACTCATACACACAAGCACAGAAGCAGGAAAGTAGGAGGCGGAAACGAGAAGGCAGCGGGGTTTGTGGATATGATCTCAGTAC
ATCCTATACATGTTTAAGATCATGAGAAAATATACATTTTAATTAAAAACTAAATTCCAACATATGTAAAAGATTTTCCAGTATGGAGTTTAGT
AAGTCTGCATGCCTGAAAGTAGAAAGAGGGAAGATTAATGGCTTGAAAGAGGTCTTAGTTTGGACTCTGCTGTGTGACCAAGGTCATTTAGTGT
GTAGTGTAAAGATCAAATCCAAAACCTTCTGGTGAAGTGGCTTGGCAGTTAAGAGCACTGATCTTCCAGAGGACCAGTCAGTAGGTTTAATTCC
TAGAACCCCTCACAGCTCACAACCACCTGTAACTTCGGTCCCAGGGAATATGATGCCCTCTTCAGGCTTCTGTGGGCACCAGGCATATAAATGG
TGCACAGATATACATGCAGGCAAAACATCCATACACACAGAAAGAAAAAACCAAAAACCTAAGGCCTTGTCATGCCACACAAGTGATCTACTAC
TGATCCCACGGAAGGGCCTGGAAACCCACCAGCTATTACCTTTGGGAACTGCCTTCACTACAAAGAGCTCTCACCCCCATTTCCATGGTAGTCA
TATGAATGCCTTTCCCATTGCTGGGACCTTCAGATCCTGGGAGACAGGCTAAAGTGATCTGGGCTATCTCCCTCACACAGCTCAAGGTACCCCA
AATAGACATCTTGTATGTCAGTGTCCATATGACAAAATATTCCAAACTTACAATGAAGAAAATGGAGGTCCAGAGAGCGCAACTCAGTTGTGGC
TAAGGTGCATCTGTGTTTATATCCAGATACTTAGCGCCTAGGCTTGTGAGTTAGAGAATGATTTTATTGCGACAAAAAAGATGATAATGACTGA
ATCTATTAGTAGGGATTTGGTATAAGATTAAGTAAAATATCATTAATGAAGAAATCATTAATTATTTAAATATATAATACAAGCTACTTAATAA
TACTTAAATTATTTAATAAATATTCTAAGTGCCTAATCATCAATTAATCGGAATCCCTTTCTACCAAAAATTCCATATGTATGAAAATTTAAAC
CATACGAATTTTAGACATACCACCAATCTTAGACAAACACCCTTGACATCGAGAAGGCCCTAATCGAGACTTCATTCTGTGATGTAAAAGCAGA
GCAGATTCTACCTCTTAGAATGCATCACAAATGAACAGAAACTGGTTAAACAGCAAATTCTGTTTTCCATTACAGTACCCTCTCTCCACCCTAA
CACACCCTTATTCTGCGTTGGAAACTTTAAGACAAAACTGTCTTGCTTTGGTGTTTCGTAATACTTCCTGGGACAGCCATGCCACCATGCCCCA
GCCAGGACTTTCTATGGCTGTTAGCTGTCCGTCTTAAAACATGGTGCTGAAGCCCTTCCCACAGAGAGACACCATTGTTTCCTGGGTTTAGGCT
CCTGAGTCCAGGCCACCTGTGGCAAGGCAGGAACGATGCTGAGTGACTTCCGAAACTAGGTCCGAATTTCTGCCAACCTTCCCGGAACTACCTT
CTCCTGGAACTCAGTCACCCTGTTCAGAGGGTGCCAAGCAGCCATGAAGAGAAGGCCAGTACAGGTGTTCAGCCAACAGCCTTAACCGAGGTCC
TAGCCAAAGCTTGCCAAGCCGACGGCTGCCACGTGACCCTGCCTAGCCTTTAGGCTGTCTCAGCAGATGCTGACCACAATAAACATGGGCTGTT
TTGCTGTTCTCAAAGGACAGATTTGAAAGCAAATTAAATACTAGTGGGGGTTTTAAGCCCTGGGTGTAGAGTGGACACCACCATTCTCCAGGCA
TTTGAAAAGACAGCTTGCCTACCATAAGTTCAGCAGAAAAGATACTGACTGGAGAGACGC
MOUSE SEQUENCE—mRNA
AGCGGCCGCCGCGGGCGCGCTCTAGCAGCGCAGGCCGGAGCTCAGGGCCCCGCGCGCCCGGCCCGCCCCGCGCTTCTCCGCCCGCGCCGCAGCC
ATGGCGCGCCGCTGACCCGCCCGCCCGCCCGCCCCCGCCCCGACCCGGCTCGGCTCCCGCCGGTCCGCGCCGCTCCGCACCGGAGCCCGCAGGC
GAGGAGAGGCCGCGCGCATCTCCAGGGTACCCTCAGAGGCCAGAAGAGGGTGTCAGAGCCCTTGTAACTCGAGTTTGACGGTCGTCAGCTGCGC
ATCTTCACCATGGCAGACGATGATCCCTACGGAACTGGGCAAATGTTTCATTTGAACACTGCTTTGACTCACTCAATATTTAATGCAGAATTAT
ATTCACCAGAAATACCACTGTCAACAGATGGCCCATACCTTCAAATATTAGAGCAACCAAAACAGAGGGGATTTCGATTCCGCTATGTGTGTGA
AGCCCCATCACACGGAGGGCTTCCGGGAGCCTCTAGTGAGAAGAACAAGAAATCCTACCCACAGGTCAAAATTTGCAACTATGTGGGGCCTGCA
AAGGTTATCGTTCAGTTGGTCACAAATCGAAAAAACATCCACCTGCACGCCCACAGCCTGGTGGGCAACCACTGTGAGGACGCGGTATGCACCG
TAACAGCAGGACCCAAGGACATGGTGGTTGGCTTTGCAAACCTGGGAATACTTCATGTCACTAAGAAAAAGGTATTTCAAACACTGGAAGCACG
GATGACAGAGGCGTGTATTAGGGGCTATAATCCTGGACTTCTGGTGCATTCTGACCTTGCCTATCTACAAGCAGAAGGCGGAGGACACCGGCAA
CTCACAGACAGAGAGAAGGAGATCATCCGCCAGGCAGCCGTGCAGCAGACCAAGGAGATGGACCTGAGCGTGCTGCGCCTCATGTTCACAGCCT
TCCTCCCTGACAGCACTGGCAGCTTCACTCGGAGACTGGAGCCTGTGGTGTCAGACGCCATCTATGATAGCAAAGCCCCGAATGCATCCAACCT
GAAAATCGTGAGAATGGACAGAACAGCAGGATGTGTGACGGGAGGGGAGGAGATTTACCTTCTCTGTGACAAGGTTCACAAAGAAGACATCCAG
ATTCGGTTTTATGAAGACCAAGAAAATGGCGGAGTTTGGGAAGCATTTGGGGACTTTTCCCCCACGGATGTTCATAGACAGTTTGCCATTGTCT
TCAAAACGCCAAAGTATAAGGATGTCAACATTACAAAGCCAGCTTCCGTGTTTGTTCAGCTTCGGAGGAAATCAGACCTGGAAACTAGTCAACC
GAAACCCTTTCTCTACTACCCTGAAATCAAAGACAAAGAGGAAGTGCAAAGGAAACGCCAGAAGCTTATGCCGAACTTCTCGGACACCTTCGGC
GGCGGCAGTGGAGCGGGAGCCGGTGGTGGAGGCATGTTCGGTACTGCCGGTGGCGGAGGGAGTACCGGAAGCCCTGGCCCAGGGTATGGCTACT
CGAACTACGGATTTCCTCCCTACGGTGGGATTACATTCCATCCCGGAGTCACGAAATCCAACGCAGGGGTCACCCATGGCACCATAAACACCAA
ATTTAAAAATGGCCCTAAACATTGTGCCAAGAGTGATGACGAGGAGAGTCTGACTCTCCCTGAGAAGGAAACTGAAGGTGAAGGGCCCAGCCTG
CCCATGGCCTGCACCAAGACGGAACCCATCGCCTTGGCATCCACCATGGAAGACAAGGAGCAGGACATGGGATTTCAGGATAACCTCTTTCTCG
AGAAGGCTCTGCAGCTCGCCAGGCGACACGCCAACGCCCTTTTCGACTACGCAGTCACGGGGGATGTGAAGATGTTGCTGGCCGTGCAACGCCA
TCTCACCGCCGTGCAGGATGAGAATGGGGACAGTGTCTTACACTTAGCCATCATCCACCTCCACGCTCAGCTCGTGAGGGATCTGCTGGAAGTC
ACATCTGGTTTGATCTCTCATGACATCATCAACATGAGAAATGACCTGTATCAGACACCTCTCCACTTGGCCGTGATCACCAAGCAGGAAGATG
TAGTAGAGCATTTGCTGAGGGTTGGGGCTGACCTGAGCCTTCTGGACCGCTGCGGCAACTCTGTCCTGCACCTAGCTGCCAAAGAAGGACACGA
CAGAATCCTCAGCATCCTGCTCAAGAGCAGAAAAGCAGCGCCCCTTATCGACCACCCCAATGGGGAAGGTCTAAATGCCATCCACATAGCTGTG
ATGAGCAATAGCCTGCCATGTCTGCTGCTGCTGGTGGCTGCCGGGGCAGAAGTCAATGCTCAGGAGCAGAAGTCTGGGCGCACGCCGCTGCACC
TGGCCGTGGAGTACGACAACATCTCCTTGGCTGGCTGCCTGCTTCTGGAGGGTGATGCCCACGTGGACAGTACCACCTATGATGGGACTACACC
TCTGCATATAGCGGCCGGAAGAGGGTCCACCAGACTGGCAGCTCTTCTCAAAGCAGCAGGAGCAGACCCCCTGGTGGAGAACTTTGAGCCTCTC
TATGACCTGGACGACTCTTGGGAGAAGGCTGGAGAAGATGAGGGAGTGGTGCCAGGTACCACACCCCTGGACATGGCTGCCAACTGGCAGGTAT
TTGACATACTAAATGGGAAACCGTATGAGCCTGTGTTCACATCTGATGATATACTACCACAAGGGGACATGAAGCAGCTGACAGAAGACACGAG
GCTACAACTCTGCAAACTGCTGGAAATTCCTGATCCAGACAAAAACTGGGCCACTCTGGCACAGAAGTTGGGTCTGGGGATATTGAACAATGCC
TTCCGGCTGAGTCCTGCTCCTTCTAAAACTCTCATGGACAACTATGAGGTCTCTGGGGGTACCATCAAAGAGCTGATGGAGGCCCTGCAACAGA
TGGGCTACACAGAGGCCATTGAAGTGATCCAGGCAGCCTTCCGCACCCCGGCAACCACAGCCTCCAGCCCCGTGACCACTGCTCAGGTCCACTG
TCTGCCTCTCTCGTCTTCCTCCACGAGGCAGCACATAGATGAACTCCGGGATAGTGACAGCGTCTGTGACAGTGGTGTGGAGACATCCTTCCGC
AAACTCAGCTTTACAGAGTCTCTTACTGGAGACAGCCCACTGCTATCTCTGAACAAAATGCCCCACGGTTATGGGCAGGAAGGACCTATTGAAG
GCAAAATTTAGCCTGCTGGCCGTTCCCCCACACTGTGTAAACCAAAGCCCTGACAGTCCATTGCATCGTCCCAAAGGAGGAAGGCAAAGCGAAT
CCAAAGGTGCTGGAGAATCGCCGGCCTGCAGGGTCACTCGATTTCATTCAAGGCCTTCCGAATTTGGCGTCCTTCTTGGTTCTGAAATGAAATG
TAGTTGCCACGCACAGACGGTGTCTAGCAATCATGGCGCTCGCTCGCTCAGGTGCAGTCTATGGCTCAGGTGCAGTGTCTTGAGCTTTCTCTGC
TGCTACTGGATCACATTTGCTTTGTGTTGTTACTGCTGTCCCTCCGCTGGGTTCCTGCTGTCATTAAAAGGTGTCGCTGTCCCCACCCGGTGTC
CTTTCTAGCCATCTACTGTAAGTTGTGCATTCAAATTAAGATTAAGGAAAAACATATTTTTAAATGAGTACCTTGATGCGCAATAAAAAAAAAG
ACATTTCTTTTTTTAATGTGGTTTATCTGTGATTTAAAAATAAAAAACACATGAACTTATCAATATTTAAAACATGCTACAATCAGTGNTGAAA
ATAGTATTTTCCCCGTTTTATGCATTTTACATTTGTAAATATGTTTTCTAATCAATACTTTAAAAGAAGAATGTTGAATTTATAAAATGCTATT
TACTTTTTTATTTATAATAAAGTACAGCACATGTGACT
MOUSE SEQUENCE—CODING
ATGGCAGACGATGATCCCTACGGAACTGGGCAAATGTTTCATTTGAACACTGCTTTGACTCACTCAATATTTAATGCAGAATTATATTCACCAG
AAATACCACTGTCAACAGATGGCCCATACCTTCAAATATTAGAGCAACCAAAACAGAGGGGATTTCGATTCCGCTATGTGTGTGAAGGCCCATC
ACACGGAGGGCTTCCGGGAGCCTCTAGTGAGAAGAACAAGAAATCCTACCCACAGGTCAAAATTTGCAACTATGTGGGGCCTGCAAAGGTTATC
GTTCAGTTGGTCACAAATGGAAAAAACATCCACCTGCACGCCCACAGCCTGGTGCGCAAGCACTGTGAGGACGGGGTATGCACCGTAACAGCAG
GACCCAAGGACATGGTGGTTGGCTTTGCAAACCTGGGAATACTTCATGTGACTAAGAAAAAGGTATTTGAAACACTGGAAGCACGGATGACAGA
GGCGTGTATTAGGGGCTATAATCCTGGACTTCTGGTGCATTCTGACCTTGCCTATCTACAAGCAGAAGGCGGAGGAGACCGGCAACTCACAGAC
AGAGAGAAGGAGATCATCCGCCAGGCAGCCGTGCAGCAGACCAAGGAGATGGACCTGAGCGTGGTGCGCCTCATGTTCACAGCCTTCCTCCCTG
ACAGCACTGGCAGCTTCACTCGGAGACTGGAGCCTGTGGTGTCAGACGCCATCTATGATAGCAAAGCCCCGAATGCATCCAACCTGAAAATCGT
GAGAATGGACAGAACAGCAGGATGTGTGACGGGAGGGGAGGAGATTTACCTTCTCTGTGACAAGGTTCAGAAAGATGACATCCAGATTCGGTTT
TATGAAGACGAACAAAATGGCGGAGTTTGGGAAGGATTTGGGGACTTTTCCCCCACGGATGTTCATAGACAGTTTGCCATTGTCTTCAAAACGC
CAAAGTATAAGGATGTCAACATTACAAAGCCAGCTTCCGTGTTTGTTCAGCTTCGGAGGAAATCAGACCTGGAAACTAGTGAACCGAAACCCTT
TCTCTACTACCCTGAAATCAAAGACAAAGAGGAAGTGCAAAGGAAACGCCAGAAGCTTATGCCGAACTTCTCGGACAGCTTCGGCGGCGGCAGT
GGAGCGGGAGCCGGTGGTGGAGGCATGTTCGGTAGTGGCGGTGGCGGAGGGAGTACCGGAAGCCCTGGCCCAGGGTATGGCTACTCGAACTACG
GATTTCCTCCCTACGGTGGGATTACATTCCATCCCGGAGTCACGAAATCCAACGCAGGGGTCACCCATGGCACCATAAACACCAAATTTAAAAA
TGGCCCTAAAGATTGTGCCAAGAGTGATGACGAGGAGAGTCTGACTCTCCCTGAGAAGGAAACTGAAGGTGAAGGGCCCAGCCTGCCCATGGCC
TGCACCAAGACGGAACCCATCGCCTTGGCATCCACCATGGAAGACAAGGAGCAGGACATGGGATTTCAGGATAACCTCTTTCTCGAGAAGGCTC
TGCAGCTCGCCAGGCGACACGCCAACGCCCTTTTCGACTACGCAGTGACGGGGGATGTGAAGATGTTGCTGGCCGTGCAACGCCATCTCACCGC
CGTGCAGGATGAGAATGGGGACAGTGTCTTACACTTAGCCATCATCCACCTCCACGCTCAGCTCGTGAGGGATCTGCTGGAAGTCACATCTGGT
TTGATCTCTGATGACATCATCAACATGAGAAATGACCTGTATCAGACACCTCTGCACTTGGCCGTGATCACCAAGCAGGAAGATGTAGTAGAGG
ATTTGCTGAGGGTTGGGGCTGACCTGAGCCTTCTGGACCGCTGGGGCAACTCTGTCCTGCACCTAGCTGCCAAAGAAGGACACGACAGAATCCT
CAGCATCCTGCTCAAGAGCACAAAAGCAGCGCCCCTTATCGACCACCCCAATGGGGAAGGTCTAAATGCCATCCACATAGCTGTGATGAGCAAT
AGCCTGCCATGTCTGCTGCTGCTGGTGGCTCCCGGGGCAGAAGTCAATGCTCAGGAGCAGAAGTCTGGGCGCACGCCGCTGCACCTGGCCGTGG
AGTACGACAACATCTCCTTGGCTCGCTGCCTGCTTCTGGAGGGTGATGCCCACGTGGACAGTACCACCTATGATGGGACTACACCTCTGCATAT
AGCGGCCGGAAGAGGGTCCACCAGACTGGCAGCTCTTCTCAAAGCAGCAGGAGCAGACCCCCTGGTGGAGAACTTTGAGCCTCTCTATGACCTG
GACGACTCTTGGGAGAAGGCTGGAGAAGATGAGGGAGTGGTGCCAGGTACCACACCCCTGGACATGGCTGCCAACTGGCAGGTATTTGACATAC
TAAATGGGAAACCGTATCAGCCTGTGTTCACATCTGATGATATACTACCACAAGGGGACATGAAGCAGCTGACAGAAGACACGAGGCTACAACT
CTGCAAACTGCTGGAAATTCCTGATCCAGACAAAAACTGGGCCACTCTGGCACAGAAGTTGGGTCTGGGGATATTGAACAATGCCTTCCGGCTG
AGTCCTGCTCCTTCTAAAACTCTCATGGACAACTATGAGGTCTCTGGGGGTACCATCAAAGAGCTGATGGAGGCCCTGCAACAGATGGGCTACA
CAGAGGCCATTGAAGTGATCCAGGCAGCCTTCCGCACCCCGGCAACCACAGCCTCCAGCCCCGTGACCACTGCTCAGGTCCACTGTCTGCCTCT
CTCGTCTTCCTCCACGAGGCAGCACATAGATGAACTCCGGGATAGTGACACCGTCTGTGACAGTGGTGTGGAGACATCCTTCCGCAAACTCAGC
TTTACAGAGTCTCTTACTGCAGACAGCCCACTGCTATCTCTGAACAAAATGCCCCACGGTTATGGGCAGGAAGGACCTATTGAAGGCAAAATTT
AG
HUMAN SEQUENCE—GENOMIC
TTGACAAGCTGGTTCTAAAATGAATTCAGAAATGCAAAGGACCTAGAATAGCCAAAACAACCTTGAAAAAGTGAACAAAGTTGGAGGACTAACA
CTTCCTGATTTCAAGACTTATTGTAAAGCTATAAAGTAATCAAAAAGGTGTGGTATTATCATATAGATAGAGAAAAGATCAATGGAACAAAATA
GAGTCCAGACATACAGATACAACTGATTTTTAACAATTTGCAAAGGCAATTTAGTGAAGAAAGGATAGTTTTTTTTTTCAACAAATGCTGCAGA
ATCAGTTGGAAATCAATAATCTAAAAAGAAAAAACTTCAATCCATATCTTGTACCACTATATGCAAAACTTAACTCAAAATGTATCATAGACCT
GAATTTAAGTCCTCAAACTACAAAAGTTCTAGAAGAAAATAGACGAGAAAATCTTAGTGAGTTTGAGTCAGGCAAAGAGTATGTGGATACAACA
CTGAAACCACAATGCAGTAAAAAACTGACAAATTATACTTCATCAAATTTAAAATCTTCTCTGCAAAAGAGGATGAAAAGACAAGTCACAGGCT
AAAAAATATGTATTTGCAAATCATATATCTGGTAAAATACTTTTATCCAGAATATAAAATGAACTCTCAAAATTCAATAATAAGAAAAAAAATA
CACCAAAAGTAAACAAAACATTTGAACATGCATTTCACCAAATGGCAAATAACCACATAAACAGATGCCCATCATCATTAGTCATTAGGGTAAA
GCAAAGTAAAGCACAATGAGATACCACTAAACATATCAATTGAGACGATTAAAATAAAAAAGACTGACCATACCAAGGATTGCAAATATATGAA
GGAGCTAGAACTCTCATTTCCTGTAGTGGGAATATAAAATAATATACTCTTTGGGAGAACAGTCTGCAGTTTCTTAAAAAGTTACATGTATGCT
AACTATACGCTCTAGCCATTTCACTTGTAGGTATTTCCCATGACAAAAGAAAGTATATGTCTATATAAACAGTTAAATAGTTACACACAAATGT
TTTAGTGGTTTTATTAATATATCCCAAAACTGGAAACAACCTAAATGTCTATGAACAGGAAAATGGATAAATAAATTGTGACTATCTATACAAT
GGAATGCTACTCAACAATAAAAAGGAATTAACTACTGATACAACAAGGTGGATGAATTTTAAGATAATCATGCTCAATGAAAGAAGTCAGATAA
AAAATAGTACCTACTGTATGATTCCATTTATATGAAACTCTAGAAAATGCAAACTAATCTATGACAGAAAGCAAATCAGTGGTTGCTTAGGAAT
GTGGAGGAGTGAGGAATGGAAATGAGGAACTGCAGAGGGCCATGAGAAAACTTTTCAGCTGATGCACATGGTCACTGTCTTTATTATGGTGATG
GTCTCATGGGTATATATATATATCAAAAACAACTCTCTCTCTCTCTCTCTCTCCAATCCCCCAACCCCATCTCTTACAACAAGAAAGTATGTCC
CTAGTATATCTGGAAGCCATCTTTTGACCAAGAGAAAAGCCAGCATGAGGAAACTGTTAACACAGGGACAGAAGAAAAGAAAATGTCTCTGAAG
GGAAGAGAGCCAGAATTGTGATTACATTAAACCTAAAGATTTTCAACGATGTGACCTGATGAAATTCCATAATGGTTAAGTCATTTTGAGTCAT
AATTTTACATCTTTTCACCTTAGGTGAAAAGCATCCTAACCAAGATAAGTATATGCTCAGCATTATATTGATAATTTATAATACGCCATTGGAA
AAAAACTCTCTTCCTGGCATTTAATTATATTACTTATTTCAAAGAAAATGCAGTAAAAAACTGATCTGTATATCTACTGATATGTCTATCAGTA
AAACAGATAAGTATATCTATGTATACCTACTATGCATAAGCCTTACAGGTGCTACATAGAGGTGATATCTGTTGTTTATACTCTTTGTCTTCCC
TTCTTCCTTTTTTTGATAACAGTGCCCCAATTTACGCTTAGCTAACCATAATCTAGCATTACTAATTCATGTCTTCCAGATGAGATAGACCCTC
CTTGTCACTAAAAGTAGACATGTGACTCACAGCATTGTAAGAAGATAGTAATGGAACACAGGTCCCCAAATTTACCCTCCCAATCTCTTCTCAA
TTACTTTCCCCAACAAGAAAAAATGCTACTGCAGGTAAGCAGGGGCATCTAAAGCAGGGTCACTTGAGGTCACAGGCTCCAGGGTCTCTAAAAG
GAAAGAGATACTCTCACTCTTCTCTGTTTTAGGTAGTCTGGAGGGCCTTGTTATAGAAAGACTTAAAACAAAGGTCTCAATTTTATGGCTTCCA
GTGAAAGTTAGCCTGAGGAAACCTATGAATGCCTTCAGGAAAGCATTACATGGGCTTATTTGTGTGTGTTCTTGGGAAGGTCAGACTAATCTGG
GTAGTGACAGGTCATATGGAGGAACGCTGCACTCATGGAAGAGTTGCTTGTAGCCCCTGAGGTTAGGCAAGCATTCTCCCTTGCACCCAGGGAG
CTCTGGGAGAAGAAAGACTTTTTTTAAAGCTTCAGCTGGGTGTGGACAGATTGCCAGTTGGTTTACTGCCAGCCCACAAAAGCAGAAACCTTTC
CCCTGGCAGGTTTGCCACTGCATAAAAGACCCTAACTACTTGTCCTGTAATTATAGGTCAGTGTATACACAGCCTGAGGTCTGAACAGGAAATG
GAAAATAATAACTTGCCCTAGTAGTACTAGCAAGCCAGAGAGGAAAACTAAGATAAACACTTCAAACAGGTGTTTTGTTGAAATAGAACTGTTA
GAAGAGCCAGGTGAAAACTTTTCTAAAAAGTAATAAATTCACTCAAGAAGATATAATTAAAGATTAGGAGTTAAATATATGATAGATGCTAGAA
TGATAAAATATACATATTTTAAAAATAGGTAATGACTCCATGTGTTTCTGACAGTATACCCTTCCTGGTTGAAATAACTAAAATGAAATAACTA
ATGGAAATAACTGAAATGCTACATAACATATTTTTAAAAATTAATACTGCTTAGCCAGCATGAAATTCAGGAATCTAAATATTTCCAAAACTGA
AAAACTGCAGGGAATAGCTGAGAAATTGACTTAGTCTGTTTTGTGTTGCTATCACAGAAAACCACAGACTGGGTAATTTATAATGAAAAGAAAT
TTGTATCTCACAGTTCTGGAGGCTGGGAAGTCCAATATCAAGGTGCCAGCATCTTGTGAAAGCTTTCTTGCTACATCATCCCATAGTGAAAGGC
AGAAGAGCAAAAGAGCAGACACTCAAGAGACAAAGTAACCTACTCCTACAATAATGGCATTCATCCATTTATGGGGGTAGAGACCTCATGACCC
AATCAGCTCTCAAAGGTCTCACCTCTCAACACTGTTGAATGGGGAGTTAAGTCCCAACACAGGAACTTTGGGGGACATGTTCAAACGATAACAC
TGCCTATCTAGTCCTTGTCACTGGACAGAAAAAGAAGAAATTCTCACCTGTGAATTCATAACCACTTACACTATATGTGATGATCAAAAGCACA
CAAGTAAAAAATTTTATTTAAGGTTTTTCCAAGTTAATAGTATACCAGATAACAGGCAAAAAGAAGCACAAATATTCCTTACTCAATTTTAACC
TAAGCTTCAAATAATTTCCAGAGATTGTTTCAAGAAACGTAAGCAATTTATTATTTATAATTCAGTTGACATATTAAACAGCATATGAAATATA
GCTGAAAAGAGAATTGATAAACTAGAAGGCAAATATAAAGAAATTATCCAGAATGAAATCCAAAGAGATAAGAGATATTAAAAGACACAGAGAT
TAGATTGAGAAGATCTAACATATACCTAATTTAAATTCCAGAAAACAAGTCTAGGGAAAATGAAAGAGAGGAAATGTTACAAAAGATAATGGAC
TAGGAATGTAACTGTTGAAACATCAAATCTTGAGACCCAGGGAGCCATATAACATGCTTCCAAAATCCAAGCTTATTATATTAATATACAAAAA
CATTAATATTAATATTATATATTAATGTTATTGTTAATATAATCATTATATTATTAAACATATAATTTATACATAAACAATATATTATATATAA
ATGTATATGATATATAATATACAATAGTTATAATATATAATAAATTATATATTGACTTCTATATAATATAGAATTATATAACTTATAAATTATA
TATAGCTTTATATATAAATCAATAATTATATATTGATGTGTACAATTATTATTTTATATTATTATTAATAATTATATAATTGGTATTATATAAA
TATTAATTTTATATTGCAATAGTTATAAAGTAAATATCATATAACATTATTAATATAATAAATATAGTATATTAATATTAATAACTTGTATTTA
TTAACAATATATTAATAAATGACATAATTTCACATTTCTTTCAGATTTCTGTAAAAGCTTATCAGGAGTTATATATATGCAAAACCATATTTAT
CTTATTAAAGAGGCATGTATAAGTGTCAGACCATCACTTATGAATAGATTTGACAGCCAGATATACAACCTTTCAGAAAACAGAAGCAAACCTC
CTCCATAAAAACAAGTAATTGGCAGCTGTCTCAATGATGTCCAGTAACTAGCCTTATTTCACTCAGAACAGTTTTGTCGGCTTCATATAGCATG
ACCAATCACCCTGGTTTACTAGAGCCTGAGGGTTTCCTGCGCAAGGGACTCTCTGTGCTAAAAGCAAGATAGTCCTGGATCAACCGGGATGAGT
TAAGTCTCTATTTCCATGCCTTCATAGAAATCATTAAAAGATATCTCTCTGTTATGGGCTGAATAATGTCACCCACCCTGAAATTCCTATGTTG
AAGTCCTAACCCCCAGTACCTCAGAATGTAACTGGATTTTGAGATAGGGTTTTTAAAGAGGTATATAAGTTAAAATGAGGACAGTGGGATGGGC
CCTAATCTAATATGACTGATGTCCTTATAAGACGAAATGATTTGGACGTGGACACAGAGGGACAACCACATGAGGACAGGGAGAAGATGGCCAT
CTGCAAGCCAAGGAGAGAAGCCCTCAAGTAAACCAATGCTGCCAACACATTGCTGTTGAACTTCCAGTCTCTAGAACCATAAAAAATATTTCTG
TTGTTTAAGCCACCCAGTCTGAGATATTTTGTTTTGGCAGCCCTAACAAACACATTCTCTAAGGGTTTATTCTCATTTTTGGTTTAGTGTATTT
CAGCTGATGATTATAGCCTAATCCTCCCCCTCTTCTTCGGGCCTCTCTTCTCTGTACTCTTCCTCCTCATATCTCACACAGGCAGATTTGTTCC
TGCCTCCTCACCTACATTACGAGCTCCGCACAGTCTCCTTTCTTCTGGCTCCGGCTCCCTCATTAATCTTTCTGTTATTAACGCTATTAACTCG
TTAAACTATCTCTCCACCTTTCCAAACTTCCATTAACTCTGGCACTCCATAAGTGTTAGTCAAATAAATGAAGAATCACACTAACTCCTCTTTC
CTTCCCTCTCACTCCAAACTCTTGGTGAGATTCTTTTGCACTTATGCCCTCGTTTCCCCATTTCCCACTCACTTGTCAGTCTCCTCATTCTTGG
TTTTCCTCATTATATTGAAACACATCTGAAAAACTCATGTAAGACCACAGCCACTGCGTATCAGTGCTATTTGAGAGTTAATAAAGTTCCCTTT
TCCTCCAAGACAGCGAGCAGAGTGAACAGAAGAAAGACTGGTTTCTGCCCTTGGCTGCACAGCCATGCCCCGAGGCCGGGCTTAGGATTTTATA
ATTCACAAAGTCAACTGTCTCTTCCCCTCTCCATCCTTTAGGAACTGTCTGCAGTATCTGACACTGTTGAGAGCTCTCATACCCTTTAAACTCT
CTCTTCTCTGGCTTTCTGAGCTTTCCTCCCACTTTTGATGGCACTGCAGGAGTTTTTAAGCATCTCTCTCTCATACCAACCCCAGAAATAAAGT
CATTCCTCGAATTCCTGTACTTAGCCCCTTGTCTCCTAACTCTTTGACCACTAATGTTTTAGAAACACTCTCCTGACAACTATCGCATCTTTGC
AGTTGTTTCCATTTCCAACGCCATGTCTCTCTGAGCAGCAGCCTTGAGTTTTCAATTGCATATTTATTAGTCATTTCCATATGGACATCTCACT
AAATCTGTCATACTAAACATGTCTAAAATATGTATGTTTACATGTTACATGTATGTTTGGTATGTGGGAATGCATTACATTCCCACAGATTTTT
TCCCAGTTACAGTGTTACTATTCTAATCACTCAGACTAGAACACTCACAGCTATCTATGAGTCTTCCTCTCACTCAAACTGCATATATAATCTA
TAAACAGGTCCTGTTGACCTCATCTTCTAAAAATCTCTTACATGCTCCTTTTTATATCATTGGATAATAGTTTAGTGTGCTGCTTAATATCATG
CACACTGGAGCCAAATCTGTTAGGTTCAAATCCTCACTCTCCTTCTATTTACTAATTATGAAACCTTAGGAAAATTATTCAACTTTTCATCTGT
AAATATGGGGATTATAATGGTGATCCACTTTATATGACTGACAACCAAATAAGTGAATACGTGTATAGCAGCTATTAACCATATGCTTAGCCGA
CCTATTTCAACCTCCTAACCAACCAGAAGAACTGTAGTTCCCTGCTCTCTTGCATCTAAGTGGGGCCATGGAATGTGGGCAGAAGTGTTGTGTC
ACCTTTACTCAGACGCAATTAAAAAGAAGACATACTTTTCCTATGTTCTCATTGCCAATGAACACACTACACAGATCTCAACCTCAGAATTCTC
TCCACACTGGTATTTCCAAAATAGCTTCATGAGAAGGGAAATTTGTGTAGTTTCAATGATTTAGTGATGCTATGGTTTGGGTATTTGTCTCCTC
TAAACCTCATGTTGAAATGTGACCCCAATTTTGGAGGTGGGGCCTAATGGGAGATGTTTCGGTCATGGAGCTGGATCCCTCACAAATAGATTGA
TGCTATTCCCTCCAAGGAGGAGAGGATGAGTGACTTCTCACTGCATTAGTTCCTATGAGAGCTGGTTGCTAAAAAGTTTGGCATCTCTCCCCTC
TCTCTCGCCATACGATCTCTCTGCACACACCACCCGCCTTTGCCTTCCATTGAGTGGAAGCAGCCTGAGGCCCTCACCAGACGCATATGCTGGT
GCCATGCTTCTTGTACAGTCTGAAGACTGTGAGCCTAATAAACCTCTTTTCATTATAAGCTACCTAGTCTTGTGTATTCTGCCATAGCAACACA
AATGAACTAAGACAAGTGACAAACACAAAATAACTTGTCTTTTAGACAATATTGATGTCTGAAATTCCACATTGAGGCATTTTTTTCCTCTTCG
TAGCCTGATAATGGCAAATACCTTCTGACACCTTAGGATGGATGAATTTGTAAGAGATATTCCTGGAAGCAGGTTTGTACAGTGTAGAGAAAAT
GTTGGGTAACAAACCAGGAATGGAGGACGGTTAATCAGCAAGCCAAAAAGCTTTTATAGTTTTGCCTGCTCTGCTGCATGGAATTACATCCCCC
CAAAACATATGTTCAAGTCCTAACCACAGGTACCTGTGAACATGAGTTTATTTGGAACTAGGATCTTTGCAAATGTTATCAAGTTAAGATGAGG
TAATACTGGATTGGGGTGGGCTCTAAATCCAATGACTGGTGTCTTCATAAAAGAAAGGTGAGGGAGAGTTGAATATAGCGACACAAAGGAAACA
CGGGGAAGGAGACCATGTGACAATGTAGGCACAGAATGGAGTGTTGCAGCCAAGCCAAGAATGGCAAGGATTGCTGGAGCTACCAGAAGCTAGG
AGGAGGCAAAGGATTCTTCCCTAGAGCCTTCAGAGGGATCATGGCCTTAATGGCACCTTCAGTTCAGAATTTTAGGCTCCAGGACTGTGACAGA
ATAAATTCCCGTTGTAAGCTGTTCAGTCGGTGGTAAATTTTTACAGCAGCCCCAGGAAAATATGCCTGCTCAACACCCTACCCTGAATTTTAAG
TTCATTTTATGGAATCATTTTGAGTTTGCAGTTTGATTTAATCCTTTTCAGTAATTAACGCTCTTCATTTTTTACTCCCAAATATGTCACACTG
GCTCTACTCTTATAACAATTGTATTTATCTATCTTTTGGTAATGGGGCTGCCACCCCTTGCAGAATGAAAAGTAGAGTGTGTATGCTCACACCT
CCCTCCCTCCCTCCCTCTCTCCCTCCCTCTCTCTTTCTTTCTTTCTCAGAGTATCGCTCTGTCTCGCCCAGGCTGGAGTGCAGTGGCGCAATCT
TGGCTCACTGCAACCTCTGCCTCCAGGGTTCAAGCGATTCTCCTGCCTCAGCCTCTGGAGTAGCTGGGATTACAGGCGCCCGCCACCACGCCCG
GCTAATTTTTTTGTATTGTTAGTAGAGATGGGGTTTCACCATATTGGTCAGGCTGGTCTCAAACTCCTGACCTCAGGGGATCCACCCGCCTTGG
CCTCCCAAAGTGCTGGGATTACAGGTGTGAGCCACCGCACCCGGCTCACAGAATTTCTTAAAAAGTCATTGTCTTCAGTCTTTATGGATCCTCT
AAATTCCAGCTGCTCAGCCTCCCGGAGCTAAATGAGATCACTTAAAGGTCGACTGTTGGGGCCTCCAAGGTCGCCTCGGAGACTCACCTCATAG
GCAATCAAATCCATCGCTGGGTTTTTGTGTGTGTGGGTTTTTTTTCTTTATTTTTTTTTTCCAGAAAAACACTCCACCAAGAAGGTTTTTATAA
CCTACTGGAGGAGGAGGATGGAGAGTGGGCACGAAAGGACTAGGATGAACATGCCATAAGCAATCTAAATGCCACTGCAATATTGCCTGGTGAG
GACCTGATTACTGATGTTTTAAAGCTAAGCTTTCAGTTGTCACTCCACCCAGTAGTGAAACAATGAGCTCTAAAATATATATTTCGGCTCAAGC
TTTCTTATGTGGGGAGGTAATCCACCCGAAGGTATCCCCAGCCTGTACCTAATACAGTGCCCAGCACTAAAGCAGCTCAGATGCCAGTGAATGG
TGGCCACTGGGAGGCCTCTCAGTGGGTGCCAGTACCGGTCTCTTCAGAGAAAAAGAAAACTCCCCTCTGCCAGATCAGTATTTTATGAGCTGTG
AACCAAAACCATTGCTACCACCATCACTATAATTCTATCCACAGTAATTATCATAAAGGCCTAACAATGCCTTGTAGATGAACATTCTGAGTAA
CTGCTCTATAACCAGGAGATTTAAGACCGCACCAAAAACCAGTAGAGGGTTATACTTTACTGGGCACAAGTCGTTTATGATAACGAAATTGTAG
TTTAATCTGTGAAGAGATGTGAATGTAACTGAGACACGCTTAAATGGAATATACAGATGAGCTTTATTTTTATATCTGGCATGCTTGGATCCAT
GCCGACCCTCCAGCTGCTCCGGCCTGCCCTTAGGGCCTATGGACCGCATGACTCTATCAGCGGCACTGCCACCGCCGCCGCCTCCGTGCTGCCT
GCGTTCCCCGACCATTGATTGGGCCCGGCAGGCGCTTCCTGGGGGCTTCCCTACCGGCTCCAGCCCTTGGGATTCGGGAGCGCCCTGCTAGGAA
GCCAGAGCCCCGCAGGGGCCGCGGCGTCCAGGCCGCCTAACGCGCGCCCCTCGCCCGGCGCCCCGAAGCGGCCCCGAGGGGCGGGAGCCGAGGC
GAGCGGCAAGGCCGGGCCGGCGGCGCACAGCGCCCCTACAAGTGCGGGCTTCCCCCACCCCCGGCAGCGACCCTACCTCCCGCCCCCGCTGCGT
GCGCGCGTGTGTCCGTCTGTCTGTATGCTCTCTCGACGTCAGTGGGAATTTCCAGCCAGGAAGTGAGAGAGTGAGCGAGACAGAAAGAGAGAGA
AGTGCACCAGCGAGCCGGGGCAGGAAGAGGAGGTTTCGCCACCGGAGCGGCCCGGCCACGCGCTGACAGCTTCCCCTGCCCTTCCCGTCGGTCG
GGCCGCCAGCCGCCGCAGCCCTCGGCCTGCACGCAGCCACCGGCCCCGCTCCCGGAGCCCAGCGCCCCCGAGGCCGCAGCCGCCCGGCCAGTAA
GGCGGCGCCGCCGCCCGGCCACCGCGCGCCCTGCGCTTCCCTCCGCCCGCGCTGCGGCCATGGCGCGGCGCTGACTGGCCTGGCCCGGCCCCGC
CGCGCTCCCGCTCGCCCCGACCCGCACTCGGGCCCGCCCGGGCTCCCGCCTGCCGCCGCCTCTTCCTTCTCCAGCCGGCAGGCCCGCGCCGCTT
AGGAGGGAGAGCCCACCCGCGCCAGGAGGCCGAACGCGGACTCGCCACCCGGGTAAGCCAAACTCGGGCGAGTGGGGGCCCGGCAGGCGACGCC
TGGCCCAAGTCCTGCCGCCCAGCCCTCCGCACCCCCTCCAGCCCCACTCGGACTTCCTCATTCCTGCGCTAACCGCTGGGCTAGACCGTGGGAG
GAGGTTGACAGTAGCTGAGAGGCACATGGGATTACCGACAGCGGGGAAAGACACATCCGGACCTCCCACGGGCTAGTCGGCCGAAGGGCCGCGG
CCGCCCGGCGGTCATTTCTCTTCACGTCCCTCCGCGGGGCGGGAACGCGAACCGGAGGGGAACTCTTACAAACTTTTAAAATCCCCAACCCCAG
CCCCACCTGGGGGATGGTGAGAAGGTTGGAGTGCGCTGCGGCGCGGAGGAGAGAGGGAAGGTGGTTGGTGCAGTGCAGAACCAGATTTCACCTA
AGGGCGTTCCATGAAATGAAAGCAGAAGTGCTTGTCAGTCCTCTTGGCTGGGAAAGCCCTCCCCAGCTCCCCAGAATTGAATAGGAGAGTCTAT
TCATCCCTCTCCTACTTCTAAAAGAAACCTGGCTCGCTCTCTGTCTCTCTCTCTCTCCCCCCTCCCCCCCTCCGTGCGCGCGCGCGCGCGCACA
CACACACACACACACACACACACACACACGATATAGTCACCTGCTATAGGACTTGATTCTGATCCTCCGGGGCCTGGCATTTGAGAGAGAAAAT
AAATTACCTACCTGGATACTTAGAGCACTTTTTAGACTTGATTATGTAAAACTCTTGGACAGTGCCACCATGCATTATGCATGACCCCTGAAAC
AAAAATAATGTAAAAACAAGGCCTATCCTAAATGCAAGTTTTTCTATGGTCCTTAAAAACAGACACCAGGGAATGTTTGCCCTGACTTGCTGGT
TTATCCTGAAACTCAAATGGGACTTAATGATACAGGATTTAATTATCAAGATTGACATGTTCATGGACTTTGTTAAGTAGAGGTTTTCAGCATT
ACAGTTTATGAAATAACCATATTTAACTCAAAGATTTTTTTATAAAGCTCATAATCTGAAAAAATGCTTATGGTATTACTAAAACATTTAATAG
CTCTCGCATCAAAATATTCTACATACTGGTGATCTTTTAGTAAATAGTAAAGGTTAGGGTCAAGATATTCAATAATTTTTTTCTAGCACAATCC
CACAACTGAATATTGTCAAGCAGTTTAAACTGCATTCGTGTTTCTTAAAACTTTAAAAGGGAAACTTAAAACTAAAGTATGTTGTTTTTCTGAT
TTTAATATTGTGCTTTCTAGAGATGAATCCTTTTACTGTTTGAGGCACTATAGGAATTATGTATTTAAATGTATGTATTTAAATTTGAAGCAAT
GTACTTTTTTGAGTTTATAAACTTGGGAAGACAGGAAATAAAAAGATTTGTTTCCTGTAGAATTTTACATTCATGATTTAATTCAATTGCTAAA
ATGGAAAGAACATGTACGATTAATGGAGACTTGGAATCTGAGATTATTCATCCATATTGATACACCTGCAATTCCTAAATGCCCTCCCCTGCTT
GTTTAGATAAAATGTCTTCCTGGGCTGCAGCCTACTGGACAAACTTGAACACAAAGGAACACACGTTATATGTACATTTAATACTGGAACCGAA
AAGCTGCTTGCAGTGAAAAGCAAACCACTTCTAAACTCTTTGAGACTTTTTAAAGAAGGTAGTATAATCCTTTTAGGCGTTGGTGGTGATGTGA
AAATCTATTATCTTTTTGCTGAAATTTCATTCTTATGTTAGGCATTGGCACTCACTTGTGCTGAGTAAATGCCCGTGTTTTTCAAACCCAGATA
GTAAATACTGGGGGTATACATACAAAAATAGTCCTTCCCCTTTGTAAGCATTGCTTTGATCTTTCCACTTCCTTTTTACTCTACCTCTTAAACA
AGATCTGTGTTGATTGAGTTGATTAAAGCACAATTAATCTGAAATAGGCAGAATTTTAGATTTAGTCATTATATTCCTTACATTTCTGTTGTTA
CTGTTCCTGGAAACAAATTCAAGTAGTTTGAGAAACTAATATTTATGCAGCTTCTTTTAACTACACTTTTAGACTTGCAGATAATTGATTAAGT
AAGTTACTACCTTGACTTTCAGGGATTGCTCATTGTGGTAGATACGACGTCTAGATAGGGAATTGGCAGCTACATGAATGTTGCACTATCATTC
CATCAGCAAGACCTTATTTTTACCTAATTTATGAGAGGTATTTCTCTGTTCAGAAGAGGTGAAAGTTCTGGCTTCTGGGGGGAAGTGGTTACTT
CATAACCTTCAATTGGTTTGAACTTGGGAGAAGTAAGAAAAGTAGTCGATATTTTCAAACAGTAAAATAATTGTATCTGAGTTGCTGTATGGAT
TTTTGATGAAGTACTCAAAATGTCATCTTTTGCATCTTCGGGTCTTAGTAGATGTGCTATTCTGAGTATAGAATTGACTGCAGTAGGAAGTGTA
TCTGGAAATGCACAGTACTGTTGTATAACCAATAGAGTAAATTCTCAGATTATGCTCTTCTGGTCCACAGATTGATCAGAAATCACATTAACAG
GAATGGATGTCTATGCAAAATTTCTCATTAATTTTCTTACTTTTAGTAGGAAGTTTGTTTTATTCGGTTGGTCTGTTGGTTTTCTGTTTACAAG
GTGAATTGTCTACAGTAGTGGGATTGTTATATTAGAAGAGAAGGAGAAGGAAAAATAGAAGTTAGTGCTAGAGGATATGTTAAGATGTTCTGCT
CCTACATCTACATTTGTGTCTAATCAGCATTAATTGATGACTACGTCTTGGTAAACAGTAGACCATACTGGAGCAATTTACTCTGCTTTTTGGG
AATTAGTTCTCATTTGCACTCTTGCACCTCCCATAAATACTGAACTCACTTGTAGTGTTCTTAGAGGTCAGCAATAAACCATCAACACCATATG
ATCTCTCTTTGAATTTTGTCACTACTTTGGAGACAGACAGTTTGGAAGAACTGAAGGTACTCAGCCTCAGGGAGAAAGGTTTGAACGTTTTGGC
ATAGTCCTGCAAGTCAGTTTGGCTCCTTGACTGTGAATTTTTTTAATCAAAAAAGATTAATATTTGAAACCACATAATTTGGACACTTAATAGT
TCTGGTAACATAACCAAGCCTTTTATCTTTGTAGTATTTCATTAATTAGTATCCAGTGCCTCACTTTTAGAAGAAAAAAAGAGAAAAAAGTAGT
AACATTCTTTTAATGATGATGAAATTGCTTCATACCTTTGGTCTCTCCTGTATTAGGGGTTGTTCCTGGGTGCAAATTAATATACTGTCTTTTA
CATTTCCGGAGGTTGTTGACCTGGCCTTGAAGATCAGCCAACAGTTTAGAAAATAAGTTGTCCAAATTGCATTGCTACACCTCAAATAATTTAA
AGCCTGACAGTTCTGTACAGAATAAGAGTCACATAAGATGTGATTTGTTAAAGATTATAGAAATCTCAGAAGATAGATTATGGATGAAAATATT
TGTTTAATTTAGAGTACTAATAATAAAAATAGTGCATTTTACATTTTAAAAAGAAAACAATTTTTATTGTGTCATATTTTCTGTAGTAACAAAC
AGAATTAATTATCAGTAATACAAATTTCATCTGCTTTAATTTATTCCCTGTTTTTGAAATTAGAATAATGATATATTTGGTTTAATTATCCATT
ACAAATTGCCTTATTATTTTTCATTCATCTTTCACCATTAATCCAGAAACCTTAAAATAGCTTTTATTTTGACCCATGCCTTCAGTAGATATCA
ACAGACTTGAGTTCATCTATGGATCCTATTAGTTTAAACTCTCACAAATTTTGACTGCTTAAGCTTTAGAAAAGAATTTATTCTTTAAAATAGC
CAAAATTATAAAAAATGAAATAAAGAAAAAGCTGTTTGAGAGAATCAAATACTCTAGTTTAACTAAAAATAAATAATATCTAATCAATACTGAC
GAAAGAAATATCTGATGCTTAGCATTGTACTTGTTGGTACATAGTAAGCAATCATTAACTGTTAAATGAATGAATGATATGAAGTTGTTGCAAA
ATTTTTTTTTAAATGGACAGCCATCTTCTATAAGGAACAAAAATTACATTATTTTGCTTTGCCTTCTGGAAAGAAAGGAGCACAAAATAGTCCT
TGTGTCTTAGATATTATGGAAATCTTATCTAAAGACCACTTGAAGTTTACTAGAACAAATAATTTATTAAAAAGTATCTGGCGCCTTCCAAAAG
GGGAACATTTCATTCTGAAAGAGGCATTTATGAACTAGAAAAAAAATGCCTTTTAGTTTCAAAGTTTTAGAGGCACCAGAGCAAGATTATAAAG
TAATTGGAATGATAAAACGTGCTTGTAAAATTGAAAAATGTAATTTTTTGGTGAAGTTAGAATTTCTGTACACTCCATTGAAACTGTAATATCA
AATACTAATACCTCCAAAAAAGAAACAATGCTACAAGCACTGCTTGGTAATCTAAGAATTGTTAACACTTATCTAAATTAATCAACCAAAAAGT
TAAAACAAAATATGTTATTTTGTTATGTTACCTCTAGAATTAAACACCTATCACCCATAAATCTGTCACTGTTAAAAGTTACTAGATGGCAAAC
CTCAGGAACAAGCAGGCAGTTAGCAGGGTTTACTGTACTTTCCACTTTCATACTTTAATGATTTTTCCAACACTATAGAACCTGGGGCCTCATC
CTAGCACAGTTTGTTCATTATTTTTCAGTTGTAGCATTTTTTAAAAGTTGGCCTTGAATTTCTGAATTGAACCATTTATTTTTTTGTGGACACA
GTTTTTTAAATGAAATATACCATTTAGACTAGAAAAGAGCAAAATGCCAATACTCCCTGTCCTAGTGTCATTGTGCCCCCTGGGAACCCTAATG
CAGTCCTGTGGGAAAACAACCATTTTATTGAGCGCTGCTCACTTCAGTTCATTCTGCTTTGCTGCCACTTAGTTCTGGTTCTGACCCTGATCTC
CCCTCACATCACTCACAACAAACATTCAAGTTTAAAAATTAAGTTAAAAGTTAGTTACATATATATGTAACTAATGTGTATATTAAATATACTG
ATATATATTAGTTACATATACAGATATGGCAACAACAAAAAAGGTTATATATATACATATTTGTAACTAATATGTATATTAAATATAATGATAT
ATGTATAAATACATATTTGTAACTAATATAATTATATTTAATATATATTAGTTACAAATATGTATATATAATATTTTTTCTTGTTGCCAGCAAA
ATGAGAAAGAAATTAAATGTTAACATCTATAACAATTGCACTGTAAAGCCGTCATTGTTGAAAAGTACTCCGAAGATTAATAGCGGCAGTGTTT
GTTAAAATAATAAAATAATCCAGGCACTCTGCCTGTGACTTCCCCATGAGATGGCTTCCAGAAAAAGATAAATTTTTGTGTGTTCCCTTACGAG
TTCAGTCTGCAGCTTCTGGGCATGAAGACAAGCATAAACAAAACAAACAAGGCTCTAACTTTGTTATAATCCTTACGCTACTACTACAAATGTA
TGGACAATTCATGCATTTCATATTGGAAAATAAAGCTATTAGATTTTTCTTTCAGATGAACCTTTTTTTTGTAATTTATTTTTTATTTTTTATT
TTTTTTCAGTAGAGATGGGATTTTGCTCTGTTGGCCACCCTCGTCTCAAACTCCTGACCTCAAGTGATCCACCCGCCTCAGCCTCCCAAAGTGC
CGGGATTACAGCCGTGAGCCACCTAGCTTGGCCAAGGTGAACCTTTTTGATAAATGAGGCTCACTAATATTTCTAGGATTTACTTTTCACTAAC
TTGAACTAAAGAAAGTATAAGACCTGCATAAAGTTGAGCATACCTCTATAATCTCCCATTTTAAAAAGCAATTTGTTTTTAATGACTTTTATTT
GTTAAAAGCAGTTATTTCAAAAAATTATTTTTATTATTTATCATGTCATTTGCACTGAAAATACTATACCATATTTCAGTGTTAGGGCACACCT
ATCCCTCTTTATCCCAGACGTCAATTCTACCATATAACAAGTATGTAAAAAAGAAAACATAAAAGTGATGACAGACTCCCTAATCAACCTATTT
ATTGGTTCTAAGCCCAAGCCTGCAGTTCATTTAAATACTGAAAATACATTACTTAATTGTCTTAGAAAATATCTCTTCCATGACAAGAATTTTA
GGAATGAGAAAATAACTATTTTTGGATGTTTTAACACAAATTCCCAAATCATGTTTTATTGATATTTGAAGAACCTCTTTGTCTACCAGTTCTT
AAGTATAGGCTTATTTTGTAACGCTTAAACATGTATTGGTGTTTGACTGTTAAGACTTCATCTATAACCTTCAGTGTCTTTTTCTTTTAAATGG
GGTATGGACTTGGTAATGAGGCCAGAGGAGAATACCTTACAATTTATAGCATCTGCATATAATACAAGAACCTTCATGATTTATAAAATTAAAA
TGATGATGATGTGGAATGATGGAAGAAGCATGTGTGCGTGGCTATTTATGTAGAGGAAATTAGAAGTTGAAAGCAATGTTAAGAATTAAAATTA
AATTACACTTAATTTTTTGCTCAGTTGGATAGTCAGGAAAGTACTTAAATCAGCAATTGATTTACGTAAAATCAAAACTGAAAACTGAATATAC
AATCTAGGAAATAGGAAAAAAATTTTTAGTGTGTTCATACTGCTAATTTCAAACCAAGCAAAAAACCTCGGCAACTACTTTTCAGAGGAGATGG
CTTAAAATGAACCAAAAATATAGCCATTTCTCTACCCTATTCATTTAATGCATTCATTTATTATACTGCTAATACATCATAAGCTGAATAGCTT
AAGCATCGTTTGTATATGTGTATGTGATTTTAAACCTGAGTCTTTTGAAAGATTGGTCAAAGCAAAGGATAATGAAAGCATAAAGATTTAGGGG
GAAGAGTCCTCTATAAAACTAAGACTTAAACTTTGTTATCAACTTGAATGTGAAGATAGAATAATCTAGAAAGTGAATGAGAATTATAAATAAA
AGGACAAATCATATATCTCATGGTTGAGAAATACAATGAGATAAATTGTATATCTCATCTCATGGAGGGTCATAATGAAAGAAGTAAGAGAGAT
TGCCCTTGGATAGAACCTTTCGGCAGACATTTCATAATGGATTATCATATTTTTGCTAAACTGGTAACAATGGACTTTTCACCACATTATGAAA
AAACACTTTAGAAGTCAGTTGCGATCAAGCAAATATAACTACCTGGGAAATTAAAATCAAAAGCACACCAGTCCAACAATAGTCAAATGCATTT
ACTCACTCTCATACCATATCATAGCGTTAATTTGAGATAATCTCTTTATGGTGTCTCTTTATATATTTACTCGATTTCCCATTTAAGACATTTA
ACCAAATTTAAAAAGCAATCCTTAGAAACTTTAAAAATATTTCCTATTTTAAACCATTACATAATCATTTACCCTGCAGTTACAATTCTTTTTT
TGTGATGGGTGGGGAAGTATCCCATTTCTCTTAGAAAGTCTTCTGTAATAGCCAAATAGGAGTCAATCCAAGTTGCTTAGAGAGGTAGTTCAAT
CCTGTGTTGAAATAGATGACTTTTGGAGTAATTGTGCTATATTTACTGAGTATCTGTTGAACTGTAGGAGTCCAGTCATTCAAGGCAAGCTACA
GAAATGATTTTATTGCTGAGGCACTTAAGTATCTTCACATCTATATGCCAATATTAAATTTCTCATCATCCTTCATCCCCACCCCCTTGCACAG
ATTGGTATCCCCAGCACATTAGGGAATGCGGTTGTTAGCCTGTTAGGGTCCATTCAGTAGATTTCTCATGCCAGAAATCTACAACCCCTTCTTA
TACATTCAGCTTTCTTCTTCAGTCAGTCACCAAGTTCTATCAAGGCAGCCTCCTCATCTCCAGAATCCATCCCTTCCTCGTTGAATTTCTAGAT
TTTATTTTTGCATTGTCTCCTGCTTCTAACCCATCGTTCATTTCGCATAATCGTCAGCTTTCTAAAGTGTAAATCGGATCATGTCTCTGCAGTG
AATCCCAGTTCTCTTCACAAGCTCATTCTCCCACACCTCACCCCCTTACTCCCTCCGCTGTTCCCCAACCATCCCCTAATCTCAAATCCTCCAC
ACCGACTTTCAGTTTCCTATCCACAGTTTCTGACTTTGAATGCCTTTGCTTATGCCCTCCCTGTATGAGAAGTGCCTTTTCTTTCCTAGCCATC
CTGCTAACTTCTAAACCCTTAGGCCTCGTTTGAGCATCACCATTTCTCTCACTTGCCTTTCTGTTTTCTCCCTCCCCTTTCCATAAAGAGTTAT
GTGCCCCCATTCTGTGTTCTCCTGAATTCCTGTGGGTCCTAGAAAAGAAACATCTTGAGGGCAAGGAGTTCATCTTATTTACTTTTTATTTGCA
CAGCACCTGGTGTGTTTCCTGAATAAGTAAATACCGAACAAGATATCAAGAGACAGTGCTGCCAATTAACTGTGTGGCTCTGAGCAAGTTAATT
AACTTCTCTGAGTCTTATTTTTGTTCTGTAAAAAAAATGAGGTTTGACAAAATGAGCATCTCTCTCTAAAATTATGTGTTTCTTTGATGTAATT
GTATTAATTGGAAGACTTCAGATTTTCTTGCCATATCAAGATAGGTCAACTTAAGAAAAATGCATGTGAGTAAGTACCAAGCCAATTCTTGATT
ATCAGTATGATAATATGAGGAACTGCCCATTTTGTCGATAATAAGTTATAATTTTTGGTCTCAGTGTAGGTTCTTTCTCAAATCCAGATGTTTC
TGGTGTTGCATTTTTTCATTATTATTCAGTCTCTCGCATTCATATTTCTCCAAAGAAATTTATGGAAAACTTGAGACAAAAATTGAATGTAAAG
TTTAATATTTAAATGCTAGATTGATTCAAGGTCTCACTAATTATAACTATATTTGGAGGGAGAAGAAGGGGAGGTGGTGGTAGTATGAGCTGAA
TCCTCCTCTGTTCCTTCCTAGAAGGAACACAACGTCCTACATCAAGAAAAAAGACAGAAGCATATCTTGGAGATATCAAAAGAACCACCAGGTA
AACTAAAAAGAAAAAGAGTGTTAAAAAGTCAGCCTTTAAAAAGCAGGATTGGAGATGAGGAGGGGTGGAGTAGGGGAAGCATTGCTTGTTTATA
GAAGCTCTTCCATCTTCGTATAGTGTGATTTTTATTTTTTTATTATTTTTTCCTTCCTTTTTTTTGACCTCCAGCTTTAGTATAGTACAGTTGA
CAGAAATTATATATGTTTATGGTACACAACATGATCTTTTGATATACATATGCATTGTGAAATGATTAAATCAAGCTGATTAACATAAGTAGGG
TTTTATTTCTTTAACAGTGATTTATTTGAATATATGTTGAGTCCTTTCTATGTGCCAGACACAGTTCTAAGGACAAGGCACACAGTGCCAAACA
AGACAGAAATCCAGAAATTCATGGAGCTTAAATCATGTAGTCTTTTCAGTTTTTAAAATTTTCTTGCTGGGCGTAGTGGCTCATATCTGTAATC
CCAGTACTTCAGGAGGCCGAGAAGGGAGGATTGCTTAAACCCAGGAATTTGAGACCAGCATGGACAAAATAGCGAGACCCCAGTCTCTACAAAA
AATTTAAAAATTACCTGGGCATCGTGGCACATCCCTGTCGTCCCAGCTCTTCAGGAGGCCGACGTAGGAGGTTCACTTGAGCCTGGGAGGTTGA
GGCTTCAGTGAGCAGCGATTGTGCCACTGCAGTCCAGTCTAGGCAACAAAGCAAGACTGTCTCAAAAAAGGAAAAAAAAAATAGAAAGAGAAAC
TTTATTTTTACATTAGCCTTTTTTAAGTTGAAGAATCCCATAGCTAAAAAAGTACACAAATATTAATGCTGCAACTTTAGTGATTTATCTCAAG
GTGAACACGTTTAACCTAGTGGGTGCTCAAGAAATAGAACACTCGCAGCACTCCAAAAGCATCCTATCTGCCCTTTCTTAGTTAGGACCCCTTC
CCTCCACTGTCATTTAAAAGATTTCACAAACAACTTTTGGATTATATGTATGTCAATTCTGTGACTCATATATATCCACATAGACAATGGAGCG
TTGACTGAATGAATTCATTATTCCAAGGTGCTAATAATGAGCTTCACACCAGCGGGAACTCTGCTGTGTCAGTTGGGCTCTAAGCTCAGTTCAT
TTCAGATATTCCTCACTAAACAAAATACTGTTTTTCCTCAGTTGTTAGTAAAAAATAGAAAACAGATTACATTCTCATTTACTTATTACTTATG
AATGTAAAAGTAAATATTTTGCTTAATACCAAGTAGCCAGGGTCACTGACAGAGACATGGGAGAAACAAGTATTCACATCTGATGTTATCAGAC
ATAGAAGAGAGAGGTATAGTTCACCTATTTCTCCCCACTCAGTTTATACATTGACATCTGTATGTGTGTTGTTTTGTTTTGTTTTGTTTGTTTG
TTTTGAGATGTCGTGTCGCTCTGTCACACAGGCTCGAATGCAGTGGCGCAATCATGGCTCACTGCAGCCTCGACTTCCTGGTCCCAAGCTATCC
TCCCACCTCAGCCACCCAAGTAGGTGGGACTACAGGCATGCACCACCACACCTGGCAAAATTTTAATTTTATTTTACTTTTTAAAATTTTTGTA
GAGATGGGGTCTCCCTATGTTCCCAAGGCTTGTCCCCAAATACCTGGACCTCCCAAAGTGCTGGGATTATAGGTGTGAGCCACCACACCCAGCC
ATGTATATGTTAAAGCGTTTATCTGTATGAGTGGTCAGTGTAGAAAGGGCTTCATGGAGCCTGATTTTGATGGCTTGCAAATTATTATATTTGT
GTATGTGTAATCGGAAGGCCTTCGCCGTGATTATTTCTTCAAAGGAACCCTTTGTCCCTCCAGGGTGATTGTACCCATGTTCATAGGACCAAAC
ATTGCTGGTTCACATAATGACTCTTTTTCTTTTAAAACATAAAGGACATATTTTTATGGATTTTTCAGGATTAATAGAATAAAAATCTAAAGTC
ATATTCAGATTCAAGAGAACTGGATCAGTTTCTCTGTATTTGAACAAAAGAATACTAATCTCTAATTCATAATGCATGAGGTCTACTTCTTCCT
CAGTCTGAAAATATGAATATTCCAAAATGGTACTCAAGGCAGAGGCTATATATCAAAATTAAAATTCTGTGATACTATGTTTTTAGGGAAGAAG
AAAACTGTAGCATTAGAAATGTAGACGCAAAAGGTCAGAACAAAGTCCAGTATGTCAAGTCCCTTTATAATGCCATCTGAATGAAAGACCTCAC
ACCATGAACAGAGGTATTGAAGCAGTTTTTATGTGGAAATGGTAGGCAGCAATATCTCAGGAGTGACTTTAATTATAACATTTAAGGTTTTATC
TAACTTATTATAATCCTTAGAGAACTTCTCTAGCTCTACAAGTTATTCTTGGCAGAATTTGTCCTAACATAGTATTAATAAAAATAAATTTTAC
TTGAGTATGATATAAATTACTCTTAATTACAATATAATTTTAAAATGCCCAAAACATCTTTTGATCTTATGAATTTCAGTCAGCTATGATTAAG
AAGACAACCATAGGCAAAAATTTCTTTCTTATGGAACACGTTGGAATGCATTCTAATCACTTCTCTAAGAAAATCCCTGTCCCCCATGATTATA
GTGAATATGATACAGCAATAATTTAGATGCTTCTTTCAGGGGGGTCAGGTTATGTATTCTACAGTCTATTGATTCTAAGAGGCACTTTTTTCTC
CCCCAATTTAACAGTTTTGTTTAATTATATACATTTCAGGATTTTTTGGGGATATTTTTCTATCACTGGTTTCTGATTTAATTCCACTGTATTT
AGATAACATATTCCGCATGCTTTCAATTCTTTGAAATTTATTCAGACTTACTTTATGCCTCGGGACGTGGTCTATCTTCGTGAATATTCCGTGT
GTATTTAAAAATAATGTGGACTCTGGGCCGGGCGCGGTGGCTCACGGCTGTAATCCTAGCACTTTGGGAGTCCGAGGCAGGCAAATCATGAGGT
CAGGAGATCGAGACCATCCTGGCTCACACGGTGAAACCCCATCTCTACTAAAAAAATACAAAAACTTAGCCAGGCGTTGTGGTGGGCGCCTGTA
GTCCCAGCTACTCAGGAGGCTGAGGCAGGAGAACAGCGTGAACCCAGGAGGCAGAGCTTGCAGTGAGCCGAGATGGCGCCACTGCACTCCAGCC
TGGGTGACAGAGCGAGACTCCATCTCAAAAAAAAAAAGAAAATAATAATAATAATAATGTGGACTCTAAAATCATTCAGTGGGGTGTTGTAGAC
ACACTAGGTTCTTTTGATTGACAGTTTTGTTCCTAAGCCTTCTATATCTTTCCTACCATGGTTTGAATGTGTCCCCACCAAATTCAGGTGTTGA
AACACGATCACCAATGTCATGGTACGAAAAGCTACAGCCTCTAAGAAGTGATGGGATTAGGTGCCCTTTTAAAAGCCCTTGCTGGAGGAAATTT
GTCTCCTCTTGCCTTTCCGCCCTCTGCCAGTTGCGGACACAGCATTCCTCCCCTCTAGAGGATGCATCCCTCACCAGATACCAAATGCTGGTGC
CTTGATCTTGGGCTTCCCGTTGCCAGAACTGTGAGAAATAAACCTGGGCTCTTTATAAATTACCCAGTCTCAGCTATTCTGTTATAGCAGCATT
GACAGACTAAGACATTTCCTGACACTCTGTTGATTTGCTTTGATCAATTACCGAGAGAGGAATGTTAAAAATCTTAACTGTAATTGTGGATTTG
TCATTTTTTCCATTAGTTCTGCCACGTTTTTGCTTCATGTATTTTGAAGCTTTGTTAATAGGTGCGTACACAATAAGATCATTGTTTTTCTTTG
ATGAGTTGGCCCTTTGATTCTTATATGCAATGTTCCTCTTTGTCCCTGGTAATGTTTATTCTCTTGAAGTCTACTTTATCTGATATTTTAGAAT
GTAGCCTGCTTTTCTTATATTTAGCATTTGCATGATATAACTAGCCCAGTGTACCATCTTCATACATTTAAACTGTGTCTTTAATATTAAAGTA
CAATTCTTATGAAAAGCGTATACTTTAATCTTGTTTTTTAAAAATCCAGTCTGATCATCTCTGTCTTCTAGATGAGTTCAGTCCAGTTCCATGT
AATATTATTATTATTATTATTTTTTTTTTTTTTTTTTTTTGAGACGGAGTCTCGCTCTGTCGCCCAGGCTGGAGTGCAGTGGCGGGATCTCGCC
TCACTGCAAGCTCCGCCTCCCGGGTTCACGCCATTCTCCTGCCTCAGCCTCCCAAGTAGCTGGGACTACAGGCGCCCGCCACTACGCCCGGCTA
ATTTTTTGTATTTTTAGTAGAGACGGGGTTTCACCGTTTTAGCCGCGATGGTCTCGATCTCCTGACCTCGTCATCCCCCCGCCTCGGCCTCCCA
AAGTGCTGGGATTACAGGCGTGAGCCACCGCGCCCGCCCCCATCTAATATTATTACTCGTATGTTGGGTTTAAGTCTCTCATCTTGGTAATTAA
TTATCTATTTGTGCTATCTCTTCTTTATTCTTTTTTTTCTCCTTTCCTGCCATCCTTTGAATAAATCAAGTTTAGTGTTTTAAATTCCATTTTA
TCTCCTTCATTGGTTACTTTCTAAAGTGGTTACTTTACCACTTTAGAAAAGTATTTGTTTATTACAATATGCATCTTTGAGTCTCTAATTAATG
TGAGAACTTGACAATGTTATACTTCTGTTTGTCCTTCTGTGCTGTTATTGTTATACATTTTACTTCTTTATATGTTATAAACTCCAAAATATAT
TGTCTTGTTTTGCTGTGAACAGTCAATCGTCTGTTTAGGAAATTAAGAAAGAAGAAATAAAAGTCTTCTGCTTACTCACATATTTATCATTATT
GATGCTTTTTATTCTTTCATACAGGTCCAAGTGTCTATCATTTCCCTTCAGCATGAAGAACTTCTTTTTAGCATGTCTTTTAGCAGGTCTGCTG
GTGATGAATACTCTTAGCTCTTGTTTACTTGAAAATGTCAATTTCACCTTTATTTTTAAAAGATTTATTTGCTGAATATTCTGGACCAATCTTT
TTTTTTCTTTTATCACTGTAAAGGTGCCTTTACATTGCCTTCTACCTTCTACTGTATCTAATGATCATCAGAAACATCATCCTTATCATCCTTA
TCAATTGTTCCCCCATATATAATATGTCCTTTCTTGTCCTTTGCTATTTTGGGATTTTTTTTTTCTCTAGCATTAGTTTTTAACAGTTTGGCTG
TAATATGCCTAACCATGGTTTTATTTCTCTTTATCCAGCAGAGAGATCTTTGAGATTCTTGGATCTGTGGGTTGATTTTTTAAAAATCAAATTT
ATAAAAATTCTCAACTGTTATTTTTTTGAATATTTTTTCTGTCTTTTTTCTGTTTCTCGGATTCTATGTTACAAATACGTAAGACTCCTTAATA
TTGTCCTGTAGGTCACTGAAGCTGTGTTCACTTCTTTCTGCCTTTTTTTCTCTGTAATTCAGTTTGGGTAGTTTTTATTGACCTTTCTTCTAGT
TTATTGGTACTTTCTTCTATAATGTTAAATATGCTAAGTCCATCTAGTGAAATTTCATTTCAGTTCTAAAATTTCCATTCTTTTTTTTAAAAGA
AAGGTATGCATTTCTTTGCTGATATTGCCTATCTGTTCTCTCATTATGTTCATCTTTTCCTTTACATTATTGACCATATGCTATTAGCTATTTT
TGTCATTTGTGTCATTTTTTTTCTCAGTCTTATTTTCCTATTTCTTTGCACGCTTCATTATGTTTTATTGTACATTGTCGAGAGTTTGGGTTTT
TTGTCTTCCTTTAAAACGTGTTGAGCTTTGTTGTAACAGGCAGTGAATTTACTGGTGGATCACCTAGTTTTATTAAGATAGGTTTAGAGTAGCC
CTTATTTTAGGATGTTTCTAACTCCCATCGTGTGGTCTTTCTGAAATCACAACTGAATGCTCACAATCTTCACTCATCTCTTTCCACCCTGGAT
TATTACAAATTCAGTGTCTCTCAGCACTATATGATGTCTGGAATTTCCATTTAGCTCTTAGATGCCCAGAAACTGTTTTCTATTAGGTTTTGCA
GGGTCTTGTCCTACACTATCACAGCTTGGTATTTGGCCAGTGACCAGAGATGACCCCTATGTAGATTTCTGAGCCTCTTGCTTTTTGTGTAAAG
CCTTTCTTTGTGATATTCTGCCCATTGGTGTTCTCTCTTTGGGCTCTCCCTGTGTCATGATTTGGAAAGTGTCCTCAGTCAAAAAGTGAGGGCG
AATTTAGAATTTACCTAAATCACCTTATAATATATAGCCCTATGAAGTCCAATGTCTTAAATTACATCTTTAGGTATTTTGTATGGTTTCACAG
TTGAGTATAGCAAGGAGGGAGGTTCAGATAACTGTTAATCTGTGATAACTAAAACCAGAATTTGACATTCTGACAAACTTGAAATTGCAGTGCT
TCTTAAAATTAATACAGTATTACTTAAAAAACAAAAACAAAAAAACCAGTGTTTTCCAATTTCCCAATACAAAAAAGCAAAACTCCCATCTCTA
CTTTTTAGGAACTTCTATCCATTTGACAACATCATAAATATATAGGTAAACTTAGATTGCAGTAAAACTATGCTGTCTAGTGTTAAATCACCAG
TATATATAAGTTTTATAGATATTATGTGACTACAGAGCTATAGGTTATAGGAAAAATATAGGATTTCTTGGCAGAAATTGGATTTAACTAGAGC
CTTGAAGAGTGGATGCTATTGAGGTAGAAAAGATGGCATTTCAGGATGCACGCCATATTGTGAACAAATCTACAAGATCGAAATGAGCAAGTCA
TTCTGAATGATTAATCTGTCTATCATAGAGGGTTTTGATTAGGACATAGTAGTAGTTGATGCTTATAATATGATTTAATATAATAATGTAATGT
GCTGAGTGGTTGAAGTCTTTGGACTTTATCTTATAAGTAATATGGCAATAATGTGAAAGATTCTTGAAGAGGAAGAAACCCTGCAGTGAGGGAG
AACAGTACAGAGCCTATTACTGTCATTCAGAGGTGAAGCAATAAAGGTCTCTCCTAGGATGTTGGAAAGAAGGGGCAGATCCAAGAAACAACAT
GAAGAAAATAATCAGCAGAAATTGGTTATTGACTAGATATGTGTGAAAATGATATATTGGCAGAAATTGCAAAGTCAAAAATGGGCCTCATTGC
AGCAGGAATGATGAAATTCCTTTTAGATAGACAGGTGAAGGGATTTAAAACATTAACTTGTTTTAAAGGTATCACTGCTTAAAGCTAACAGTGA
TTTATTAATATTTGCACTCTGAAAGTTTTAACAGTTTTAATTTTATGTATACACCACAGTGTTTTGGCTACTATACTTGCCTCTTTTGTTCAGT
TGGGAATTGAGTAAACTAATAGAGTAAAAAAATGAGTACAAAGTGGGCTTTGACTAGAACTCTAACAAAGTGTGATCACTGAATTACCAAGATA
ATTAACCCATACCCCACATATACTCCATTTCCCATGACACATATTATATTGCATTGTTTTTGCCTATTTCCCTTCCTACTTCCTAAACTGTTGA
ATTAGTTTCCTTTTGTTGCATTAAAAAAAAGGAGAATCACCACAAAGCAAATTAAAAGATACTGATTACTTCTTCCAATTCTGTGGGCTGCCTA
GTTCATCTGGCCTGGCCTGCTTTGGCTAATCTCTGAGGTCTTTTGGCTACTTGACTGGTACTGTTTGTAGCTACTCAGCTGGTGCCCTACTGCT
GTTTGTCTCAACTTGTTTTGGCTACTCAGCTGGTGCTAGGGAGCCTCAGCTAGGACTGCCTGTCTGTGCTCCACATGGTCTTTGATTTTCCAGT
GGCTCGTGCAGGCTTGTTTGCATGGTAACAATGTTCCAAGAAAGCAAGAATGATCATTGCAAGGCCTCTTATAAGCTGGGTTTAGAACTTGTAC
TTCTACCTCTTCCTATTGGTCAGAGTATGTCCGTAGGTCTGCCAAGACTCAAAGCACAGAGAAATAGATGTTTCCTCTTGTTGAGTGGCATATA
AAAAAAAGTGTGGCCATTGATTTCAATCTCCCACAACCGTAACCTCTGTGACTACAGCAACTGCATCACTCTTTTCCACTTTGGTGTTGTTGGC
ATAGTAGGCATAAATATTTTTAGGTGAATGACTAAATTAATGAAGGAAGGAAAACATTCAGGTAGTATTTCAATATACTGTAGTTTTAAATTAT
ATATATTATATACATTTGACATAGTTTAATATATAAGTATATACACATATAGTTTTCATATATAAGTATATTATATATTATATAAAATATTTAT
ATGTTATATATAAAGTATATATATAAAATATATAGTTTTAATATGTAAGTATATGTATATAATTATATATAATATTATAAGTATATATACTTTT
ATCATACCATCAGTGAGTATAGACATGGTTCAGGGAGAATCTCTTTATTCTCCTTCTATCTTTTCAGATAGACCAAACAAAATTTTTTTTTAAA
AAAAGAACTATCACTTGAGTTAACATTGTTTGGTATTTTTATCTTCATAGGTGATTGCCTTGTGAATAACTCATTTTGTGTATTGTCAGAATCC
TATTCTGCTTTTCAGGCTGTGCTATGGGTAGACTAGCTCTAACCAGAACACTTCGGAGACCTGAAGCAAAGATACTATATTAAAATAAGTAATG
TTATATGCCAAAGAGGAAAACCACTTCTGCCTCCTAAACAACTGAAAGAACCAGAGACTGTGGGGTCAGATTAGCTAGCATTACTATATGAACC
TGAGCAAATGACTTACTCTCTTTCAGCTTCAGTGTCTTATCTAGGACATTGGTTCTCAGGATTGGTGTGAGAATTAAGAGAAATATGTGAAGTT
CTAGCTACAGTTCTTTACCCATGAGGTAGTCAGTAAAGTTTACTTCTTTCTCCATGTGAACCAAAGCACTGTCATGATGTGATGATGTTCATTA
GGGTTTCTCATGCTCAGCGTTATTGATATTTACGGTTGCATAATCCTTTATTGTGAGGGGCCATTCTGTGCAGTATAGGATGTCTACCAGCATC
TGTAGCCTCTATACACTAGATGCCAGTAGCACCACCTATCCAGCTGTGACAGGCAAAAATGTCTTCAAACGTTGCCAAATGACCCCTCGCGGCA
AAATGCAAACAGTTTATCTAGCAGTGACACATAGCCAGCCACCTTAGAAAGGGAATTGTCAGCGTACCCATAAGTGTAGAATGGGAAACAAAAT
ACTTGAGTCTTCTCACAATGTCTAAACCTTTTGCTGTCTATATTCCTCTTTTTATAATTTACCACAAACATATATTGCTTATTATGCCATCTAA
TAAAATGGCTAACATTTGCTGTGTATTATACTCGGTGTTAATAAGCTTTGTATGAACTCACTACCTGAATGTTGTATATGTATCTGCCATACCT
GTTGGATTGACATTCCACTGTAAACAGGTACCTTATCTTTACCAGCTCCAAGCACAATGTATTTGGTATATCAGGTAGCTATTCTACATGTTAA
TTGATCACATTGCACCCAAATGAAACATGGATGTGGAAGTGGAAGGACGACCTTTAAAACATGGAAAATTAGTTTATACAAATCTAAATGTTGT
AGTGCACCAAATTGATTCCTTTTTAAAAACTTTCAGAAATCAGCTTTTAAAAAAAAAAAGTAACTAAGCCATGATCAACTTTTCTTTCATCACC
ACTAAGCCAATATCAGAAAGTTGTCAATCAACAGCTCTTTTAAACTTGAAATTTTTCTATTGTATTTCTTATGAAGATTTCAGTACTGCTTAGA
AGTTTTTGGCTCATTTATCATGTTTTATTCACTGTATCTCATTAATTGTTAAAGATCTCAGACTCAAACCTTAAATTCAGGACAGATTTAAATT
TTTCCTTTTTCCTATTTAAATATATCTTTCTAATTACATAATTCAGTTAGAAAATTACAATATAGAAACATATAACTTAGTAAAAGTTTCCAGT
TACTGTATACAGATTATATTGTATACTGTATTATTTTTAACAGTTGCATATTTAACTTATAAATGTGGATAATTATAATTAAACAATCTGTTAT
CAAAGGACATACAGATTATTTCCAGTTCCTTGCTGCAGTGAATATCCTTGTACTTATTTTTTTACACACTTGCTCAGATAATTTTTAAGTACAA
ATTCCTGACGTAGAATTGCAGAATCAAAAGATACTCTCATTTGAGTGTTTCACATTTTATGCAAAGTATTTTGTTTCCTCCAGAAAATTCCTCT
GTGGAAGAGGCTTCCTCACATTCTTTATCAGTTTATTAGCCAGCAGAGAAATGATGAAAAGCCACCCCTTTTCTTGTAGTTTCATCCTCATTGC
TATCCAAGATGACGTATTAAAATCACACCTAAGTGGTGTCTGTTGTGGCTGAATATAATCTTGAAAGTAGAGACACTTCTCTCTGATTCCCAAC
TGTACTCTCTGGATGGCACCTAAAATTTTATTTTTTCAAACTGAAAATCATTTTAATGAGTTCGAAAAGCTTTTATTTTTTAAAATGAAATCCT
ATAGAAAGAATACATTTTCTTACAGTGGGTCATGGTCAAAACTCTGAATATCTCTGATCCTAGAAAGTTTCCCCAGTTACAAGGACAGGCGTGC
CCCCAACTCCATCCTTAATGGCTCAATTCTGTGGCTTTCAAGTTAATCCACTCAGTCCTACACCCTTCACCAAGAAGCTCATGTGCTACACTTT
GGTGTTAGTCGGGGCATTGTCACACAGGTTCAGAAAATAACCCATTAGGCATTTTATACAACTCATCACATGTTCAGAACTAAGCTAGTTGTAA
CATTATGTTTTTCATGTGAAAAAACACAAGCAATTCACTGCCTATTTTATTTGTTGCTAGCTAGCTATACTTTCTATTTCTTTTTCCATGCTGT
GAAAATCTTCCTAGAAACCTCACTACTGGGAAAAGAGATGCAGAGATTAGTCCCATCTTCTCCCCTGGAAATTTACTATCGCCCTTTTGGCTTC
AAGGTCCTTCCTCAAAAGCAAAGAATTGAACAAGGGAAAGTGAGGGGAAGGGCTGGGGGAACAGGGAGGTAGAAGGGGTTTTCCCTGGTCTTTT
CTAATCCTTTAGTTCCAGACGCTTAGTTGGTATTTCAACATTTTAGAAATAAAACCTGCTTTTATAATTAATGCAAATATTACTTAGTGATTCA
TCCTTTCCAAATGATTCTTGGTTTCTTTTGACATTTCATTTTTAAATTGTGAATTAACATTGACATCACCATAGCGTTACTGTTACCTCAGCTC
TTTACAGTGAACTGTATTGCAAAGGCTATAAAAGTTCAAATTTTAACTATCACCCACGTGAGCCTTGGACTGCTGTTAGGCTTTTGAAACTGTG
ATCCTTTGGCAAGAATTGGTGTATAAATCATGGGGAGGATATTGAAGGGGAAGATCATGTTTAATTTGGCAGTTGGGTGCCTGCAATTGACAGA
TTTTCATTAAGGTTTTAGCTATAGTAGTGATCATGCATCAGTTCTAATAAAGTAGGTTTCGTTTGAATTTTAAAAATTAGAGTGAGGACTGCTT
TATGTTAACTGTTTACATTCAGAAGTCTGGAAAACCATCCACAGGTTTAAATGTATATAATCAGTATAATTAACACGTCAACAGCCTCACCTCT
GTTTTTGAGGACCATAGGCTAATATTATTTATGAAGGTTCAGGAGATCAGTCTAGATCGACAGAAAGCTTAAGGGCAAAGATGGTTTTAATATT
TTTCTTGGAAGTTTGAGCTTCTGTCCTATTCTAAGGATAGGGATTTTGACATTGATTGTTCTAGCTGGTATTCACTACTGTATTTAAGTGTAAT
GTTTTTACTAGTATTCTTCTTATAACTGAAAACTATGCTAATGATTCTTTACTATAGATATATACTCATAAATACGTGTAAAAATCCAAGTTTA
CCAAAAAATATTAAGACTTAGGGGATATGTTCTTTGAATAGGCATTTATTTCCTAATTAACATATTCTAATCTCAACAAGGTTATATTCTTTTC
AAAAATCCATTCCATCAAAATCTGTCATTTTTACAGATGAAGAAACAAACGCCACAGTGAGTTTAGTTAATATCCAAGATCACACAGCCAGCAT
GATAGAACTCTTAAATTTCAAGCGACTTACCCTCTTTACTCTATCAGGTCAAATTTCCCTTTTTTAACTGCTTGTCTTAATTGAGATTTCTTTA
ATTGTGGCTGTCTGTATATCTTCAATCAATACTTTCTTGTTTTAGACAAATGTTTCCCTGCAAATCTGCATGAACTTCATGTTCTGAAGGACTC
AGAGCTGCCCTTTCCTTTTTGTTAAAGTTGTACACTGCTCTGCATCCCCCTAGAATGCTCATCAGCTTTCTCACTCTTCCTCACTCACACAGTG
CTCCAGCAGGAGAGGATTTCTTTGAAAATCAGATTCTTTTTTGGAGCCCTTTCTGCTTATTGCAGTGTTATGTCATATATTCATTACGGTGAGG
GCTAAACTCTTTGAACAAAGAGACCTGACAGTGCATTTGGGTTAAAGAACAAAGTTTATTTCTTTCTCTCATCACCACCAGCATGAGCAGCCTG
GATGGTCAGAGCACATCTGTCTCATGGCATTGTTCAGGGATCCTCATTCCTTCCCTCTCATTTATCTGCCATCCCCTAGGCTATTAATCATCAT
GTTTTTGGTAGAACCCAGGTCTTCTCCATGTCTGAGTTTTTATGGAGGAAGCATGTCAAGCGCCTTAAGCCCACACCTGCAACTGGTATGCAAG
AGTTCTGACATTGCACTGGTGAGCATTTGGTCAGATGGCCATCTCTAACTCCAACGAAGTGTAGGGTATACACTAGGTAAGCAGTCACATTTCC
AGCTACAATACTAATTCTATGGAAAAGGAGACAACATATTTTGATGGGTAGCTGACATTCTCATCTGCAGTCCTAAGTACTCATGACTGCTAGA
TAGACTTTATGAAAAAAAAAAACAAAAATAACCCCAGCAATTAATATTTACCCTCAGCCTTGTGTTTCTTTAATTTTCTCATTTTTCATGTCAC
ATATCTAAAATTTGATCTAAATAAATTTAAATAAAATTAACCTTTATTTGAATAATAATAAGGACATTTCTCAAGGAAGTAAATCTCAAATGAT
ATGTGATTTTAGATACTATCTTTATCTGACCATGTGGTTCCTTCCTTCTATCAGGGAGGGCCTCTGGGTAATAGAAGATTGCTAGGGCCTGCAG
TATATTAATGTTTAGCTGGAGTTGATTAAATCTGAGACCTATAGCCTTGATAATAAGGACTTTTATTTTTGTATTTTTATTGCACTTTTAATAT
TACAGAGTTTCTGTACCAGCTTGACAACAGCAGAAACATGGCTTGCTTCTGTGGAACACGCTAGAGCAGCAGTCCCCAACCTTTTTGGCATCAG
GGACCAGTTTCCTGCAAGACACCGTTTCCACAGACTGCGGGCTCGGCGTGATTTTGTGATGATTCAAGCACATTACATATATTGTCCACTTTAT
TTCTATTATTATTAACATTGTAATATGTAATTAAATAATTATACAACTCACCATAATGTAGAATCAGTAGGAGACCTGAGCTGCTTTTCCCACA
ACTAGACAGTCCCATCTGGGAGTGACGGGAGACAGTGACCCATCTGGAGACAGTGACAGTGATCATCAGGCGTTAGATTCTCATAAGGAGCGGG
CAATCTAGATCCCTTGCATGCACACTTCACAATACGGTTTATGCTCCTATGAGAATCTAATGCCACTGCTGATCTCACACGACACGGAGCTCAG
GCAGTAATGCAAGCGATGGGGAGCAGCTGTATATACACATCAACCTTGGCTCGCTTGCCTACTCCTCACCTCTTTTTGTCCTGCGTTATCCATA
GCCCAGGGTTTGGGGACGCCTGCACTAGAGGACAGAGCCTGGTACACTATAGCAGTCCTCCCCTTGTGTTATGCAGAGTACTGATCAGTCCCTT
TTGAGCTTCTCAACCTTGTATAAAACTTTGAGACAGTTCTGGGACCATCACAAGGTTTATGAAGGGTTAGAAAACAATACACTTAAGGAACTAA
GACTGTAAATCGTTGAAGTTCAGAGGGCGATAAAAAGAAATGGCTTCTAAGAATCCCAGTATGTATGATTTTTTATACAAAGCATAAAGATGAT
TTGTACCAAACTCAGGACACTGTGGCGAGCTTAACACGAGGGTATTGAATCATACGTAAGTGGTAATTCCTAGATTATGCAATCTCCTTGCTGT
TAGATTTAGATACCAAAGAGGAACTACATCATGTCCTCCTCCTATGGTCTTCAAAAAAGGATTATATTTGGTCTTACTGGTATAATACAGTTTA
TTCCTGCATGAATTCCATGGTGATAGAATTTTTAAAGTTCATTCTAGTGTTACAGTTTTGTTTTGTTTTGTTTTAATACACACCTTCACAATGG
CAGAAGATGATCCATATTTGCGAAGGCCTGAACAAGTAAGTGTCATAATCTCACTGATAACTTTATTTAAATATATTCATATTTCAAAAATATG
CAAAGGCAACATTAGTAAGTTAGCATTAGGAAAATTCTAAAATTAGTAAATTTTTTTTTAATTTCAGTTTCTCTGTCCTTTCATGTGAAGTTGG
AGAGGATATGTCATGTGAAATGAGTAACTTACACCCGCTTTCACACATATTGTTGCACAAAAATTGTATATGCTACAGGATAGCTATTGTATTA
GCTTTCTAGAGCTACCATAACAGATCACCACAAACCTGATGGCTTAAAACAACAAAATACATTATTTCACAGTTCTGGGGGCTAGAAGTATGAA
ATCAAGGTGTTGGCAGGGTTGGTTCCTTCTGGAGGCTCTCAGGAAGAATCTGTTCCATGCCTCTCCCCTAGCTTCTGGTTGGTTCCTCGGAATC
CTTGGCTTGTAGCTTTATCGCTCCAATCTCTTCCTCCATTGTCACGTGCGCTTCTTCCCGCCGTGTCTCTACTCTGTCTCTAAACCTAAATCTC
CTCTTTTTTTCTTCCAAAGGCATCATTGGATTTAGGGTACACCCTTATCCAATATGACTTCATCTTAATTTGTTTATATCTGCGAAGACCCTAT
TTCCAAATAAAGTCACATTCACAGGTACCGGGGATTAGCAGTTAAACCTGTCTTTTTGGGAGGCAACACAGTTTAACCCACTACAGCCCCTTGA
CATATTTATTTAATGTGTACACATGGATGTAGAGAGTGGAATGACAGACCATGGAGATTCAGAAAGCTGACGGATGGGAGCGCAGTGGGATGGG
GGTGGATGATAAGAAGTTACTTAATGGGTACAATGTATGTTATTTGAGTGTTGGATACCCTAAAAACCCTGTTTTGACTACTATACAATCTATG
CATGTAACAAAATTGCACTTGTACCCCATCAATTTATACCAAAAAAAAATTTTATTTAAAACTCATCTAGTAAGGAGACAGATTGTTGTAGTGA
AAAGATCTTTGAACTCAGAATTTGAAGATCTTTATATAAGTACTGACTACTACAGATCAGCTCCTCAGACTTGAAGAATCACTTAGTCCCCGTA
ATCCTCACTTTTCTCATTTGTAAAATAGAAATGCTAGCACCACACTAACAGGGATGAGTCAAAAATATTCAATGAATGTGCTTTGTAAGCATAA
AACTGTTCCTATCATTATATTAGTACTTATGAAAGGATAGCAGTGTCCTTTTTTCTTTTTCTCCTATATTTTATTTTGAAAAATGTTGGAAAAA
ATAAATAGAAATGTTGGAATAATCTAACAAATCTACATTGGTTCTTAAAACCGACATTTTCGCTTTTCTCTTTGCTCTCTTTTTCGTGTGTGTG
TGTGTGTGTATATATCTATATATGTATATGTATATACATAATCATTTGGAAGCAATTTTCAGACTCTGGAGCAATTATAATGGCAATATTAAAT
TATTTATGTAATGATTCCAAGAAGTATTTAAAACATGCAGATTTCTTCATTCATCAGCTATTTATTGAGGTATTACTACGTGCCAGAACATTGG
CTTAGTGGGGAAAAAATAAGTGATATATACACTCTAATTTCAGGAACCTACTATTTTAATAGGAAGAACAGAGACATTGTTTTCATATGGTGAT
ATATGCTGTAGTAGTAGTCTGCGCAGAGCGGGATAGAAATGTTAATAAGCAGCATCACTAGGTGCTACCTTGGAGGAAGACCAGACTTACGGAG
GACTTTCCGAAGGAAGTCTTCGTAAGCTGTGTTTTGAAAGATGTAGTTTGCTAGACTAGAAAAGGTGAAGCAACACTAGGTGCCAGGATAGCCT
GGTCACATTTAAATTTTACTGCCAATCATTTAAAATACTGCATTTCTGAAATTACACGTGCAAAGAATCACCCAGAAAAATAAAAACAGAAGTG
GAATATGTTTTAAACCCTCTGGTATCATTTCTATAAATAAAATGGACATATATAGCATTTATCACTTCCGGTGATATTCTGATTGATTTAGAAA
GAATGACTGCATTGCTTTTCACATTAAGATGCTCCTTTCTAATTTTATTTTTAAAATGGTGATTTAGGATAAATCTAGCAACATTTTCTTTTAT
TGCAGCAAAGACATTTGGCTAATTGTATTATCAAAACCGTTTGTTTTACCTTTCGCTTTTAATGTGCTTTCTCTAACAATTAAGCCCGAACTAA
CCAGCATGAGGATTGTGTCTGCTTGATTTTAAACCATCCTTTCCTGTCTGTACACAGGAAATCTTATCAACAAGAGATGATTCTTTATGCCACA
GAAGACTAAAACCTGCTGAAAACATTCATTTTGAAAGTTTTTCTTATGTGGGCATCTTTGCGATCATCAGTAACAGGGGTGGGGCATATTCTGA
CATTTCCATTTTTGAGGCAAGCGGTCTTGAAGTTTTTTTCTTTCTTCGCTTGAGCTTGGTGGTTTATATTTCCTAAAGTTTGCATGTCCTCAGT
GTTTTAGTGGAGTCTGGAATTTGCTCTCCACCCTGACCTAATAAACAATGCAAGCTGCAGCACAACACAACTTCTCTCTTAGTCTGAAGTGAAG
AGTATATCTCTCTCCTAAGGTGAGCCCTTTTGCCCACTCGCCAGTCATTTAGCCACCACAGGAAACAGTTTTCCTGCTACGTTATCACCTTTGC
TCTGAGACTGTTAAAATCCTGTATGAAATGAGGCCCATTTTTGCAGAACATGGTTTTTCATTCCTGTATTTCCTTTTCAGCAAACAATGAATAG
CATCAGTGAGCTTTTAATCTTATAAGCATTGAAGTGAAGCGCATTCTTTGTTCCTCAGTTGAAAATTTGCATCCCTCAAATGGATCTGAAGTAC
TGTTTATTTAAACGAGATTTTTCTTACTCTTAATTTAACTAGGCATACGAAGATCTTCAGTACCCCAGAACTTTCAATAATTTACTACTAAGGG
CAGGAGACAAACTGTAATACTGGCTACTAAAAGAAACGAAAAGCTAGTTCAGCACACTTACTAGTCTCTCATAAAAAGTAAAATATGGCTGGTT
TCCATCCTCTCCACAGACATTTCAAACATATAACTTCTTTAGGCTAAAATAAATAGCAAGCTCAGAAATACAGTTTTAAAGTCCTAAATGAGCC
AGCGGTTACAATGTTTTTTGATAAGTAATGAATAGTAATTTATTAACATAAAATGTACTTTCTTCAAAATATTCTGCATGCCCACTCTGTTGCT
GCCCTAACACATTACCACATACTTGGGGCTTAAAACAACACAAATTTATACTTCTGAGTCAGGAGCTCAGAAATCTAGAATCAAGGTGTTGGCA
GGGCTCCTTTCCTTCTTGCGGCTCTCATCTCTTTGCCTTTTGAGCTTCTAGTGGCTGCCTGCTTTCCCTGTGCACCCTCTCATCACTCCAGCCT
CTTGCTTCCATTGTCATACTTCCTACTATTCAGTGTGATTTCCTGAACCCCTCTCATCAGGCCCACCTGGACAATTCAAGACACTCTCTTCATC
TTATTATCATTACCTTAATGACATACGTAAATTTCCTTTTGTCATATAACGTAGCACTCACAGTTTCTGAGGATTAGGATGCAGACATGTTTAG
GGGGTCGTTATTCACCTTAACACACCCACTAAATTCCAGGTATTTTGTGTTATGCTAGGAAACTGAAGGTAAAAGAAATGAGTGAGCTCAGTGC
TTAGTTGTCTACTCCTTGTACTGTCAGTTATATTTTCAGTTATTCTGTAAATAGTCCCAGAGGTGCTCTCTAGCATTCTTTCAGGCCTTGACAT
TGGATAGCTAGATGAGGGGTATATTTACCACCATAGAGGAAATACTGATTTCTAGGATTATATTATTTTGTTTATACTTCTAAGAGAGTACAAG
CTTCAAATCATTCATTCAACAACTATTTAAGTATCTGTTCTGTGATAAGCATGTACTTGGCCCTCAAGATATACCAGTAAGCAAGCTAGACCAG
GTCCCTGCCTCTGGGGACTTTATAGCCAAGTTATGCTTCTCAAATCCCTTTGTTACGTAAACGTTGTCCAACCCTATGTAAAGTTTTGTACACA
TGAGAACTACCTAGGTCTCATGAAGATCTTTTAAAACCCTCATTTAAAATATTTTCACATTTTTCTTTTTGCCTTTACTTTCATTCCTATTATT
ACATTTTAGGTGTCCCAACTTCAAATTTTTCATATAATATAGGAAAAATAATGATTGAAACATTTAAATGTTCTTCTTTACAGATGTTTCATTT
GGATCCTTCTTTGACTCATACAATATTTAATCCAGAAGTATTTCAACCACAGATGGCACTGCCAACAGGTAAGAAAACTCATCCCTGTTACCCT
GTTGTTCTGCTTTCAGTCTTAGTAAAATGCAGGATTTGTTAATAGTCTGTCCTACAAACTCAGTTGTGTACCTAGAAAGGAAATGGTGATTTGT
TTTAAAAACCAATCTTTTAACATACTTTCTTGAAATATATTTGCACAAAAATATTTCATCTCAATTACCCCTTGTATGGTTTACTGCATTTCAT
ATCTGCTAGGACTACTCAGAAGGAATTTCTTTAGTCAACATAAGGATTTTCTTTATTATCTCTGTATTCCTTTTAAGTCCTTTCTCCCAGATTC
ACACGAGGCAGTCTCCACTCTTCATTTCTGTTTCAAACATTTAAGAGGGCCTGTTGTGTACTACCAGTGTGCCAGTCACTGGGGAATCAAAGTG
TACAGAAATCATAAAAATCATCTCATTAGAAAAATCATCTTTATAAAAGCTTATAACTTTGCACTCTGACAAGTTGGATAAAACTCATAATCCT
TCCTTCTACCATGACTGTATTAATTGTGCCGATTGGCAACTGTTTAATTTCGTAAGAGTAATAAAAAAAAAACTCTCTTTCTTAAAAAAAGTGA
TTTATTTGTATGTAAGCTTGGTGTGATGGCTCTTTTTATGTTCTGTTTCTCTCCTCACTTCACCCGTCACCAGTGGGACTTAACAGATGTTCAA
AAGCAGTGTGACTGAATTATGGAAGTTATGTGTAAAACAGAGTTTGCTAATCGCTGAAGGAGGCATCAGCTCTTTTATTTACCACGCACTTGCT
TGTCTTCATTGGGGGACTTTATCCCATTTGTATTATTCTCATTTAAAATTATCATCCTTACTACAGTACTTACTTTTCTTTTGTTTTAGATCAG
AAAACTTTTTCTGCATTTATATCTTTAGTATTTCTAGGAGTTCATGGGAGGGAAAAAACTAGATCTACTATACTTTCACCTTCACTAGTAAAGG
TGTATTTCAGAGGTTTTTCTGTCTTTACCCCTACAAAACATTATTTTTAGGCTGCCATGTCTGAGATCTTGTGAAGTGCTGTCCAATTTGAGCT
CTAGGTCAGCTTCCTGTGAACAAGTAAATGTCTTGCTAGTGTGTGTGTTTCTTGGGAACTTTTTAATTCTGGAGTTCAAAGTATTTTTCTTAGT
ATTAATGTATTACTGAAAATATAAGAACTTGGTTCTGTCCACAGGAAATACACACATTTTCACTTAATGTCTTCTGAATACAAAACAGTATCCT
GAGGCAGACGTTTTCTTAATTTTATTCCTTCGGATTTATGGGCATGAAAATAAGATTATCAAGATAACCATCTCAGTAAATATTCTGTGGTCAC
AGCTTTATGTTGTAGAAATTATTTTGCCATAATATTAGAAAAACTATTCAAATTACACCTAAAAATCTAATTTGTTAGTTCTTCTTACACCATC
ATTTTTACCTGTATGTTTTTCCCTCTTCATTGCAGTGTCATAGTACAATTTTTATAGTCCAAGATATATAGGATGTTGTATTCAAATTTTTAAA
GAGGTAGAGAGGTTGATTATCTAAACCAATATTAACTTGTAACTCTAAAATGGATTATTTTAGTCTTCTAGATTTTTTGAAATCTAAAACCTTT
AGTAGATCTAGTACTTGTTGAAGACTATGGGATGCGACATAACTCTTAGGTCTAAAGTTAAATTTCTCTGCACAGTGGAATCTCCTTTTCATGG
CACAGAAATGTGGCCAAAAGAATGTTGGCCTTAAAACAACAAATACATGCATATCAAATCTAATTTTCTAGTTGATTTAACTTTAATGTGTTGC
CTGAAAGAGGAAAAATGGCTCTATTTTACATGAATTTTCACATATTTAAAGGAAATTAAATCCTACAGGAAAAATAATAGCAAATTTGCAGTTG
TTTTTAGCAGAGTTAATAAAAAGTGAGAGACTTCAACTTTAATGTAAACATGCTTCCAACTCCTCTGTATTTTTCATGACATCCAAATATTGCT
CTGTTAGAGGACTGTCTTGATATGTTCTTTCACCATTTTAACAGGGAATGTTTTTATAAGTGTTACATTGTCATACAGGCTGTATCTTAGTGTG
AAAATATTTCTATTTCTAATCAATTTTATTTCCATATCCAAAATTAAATTCTAGCCATTTGGTGGGGGAATTTTTAAATTATGATTATGTTTCT
AAAAGTAATTTAAAATGTCCAGGGGTGAATTTCATACAGACAAAAGATCGTAATTTAATACATTGACCAGTGACTCCCCCTTTTTTTTCTTGTG
TATGCTTCATTTCAGATGCATTCATTTTTGCAACTCGCTTCCTTGTAAATTTCTTGAATATAACAGGAAGTAATTATACCCTTGTTTTCTCCAG
TTCTCTAGCCTTATCCTCATTTATCATAATATTGAAAAGATAAAAATAAATTCTCTGGCCGGGTGAGGTGGCTCACGCCTGTAATCCCAGCACT
TTGGGAGGCCAAGGCAGGTAGATCACGAGCTCAGGAGTTCAAGACCAGCCTGGCCAAGATGGTGAAACCCCGTCTCTACTAAAAAAATACAAAA
ATTAGCTGGGCATGGTCCTAGGCGCCTGTAATTCCAGCTCCTCTCCAGCCTGAGGCAGAGAATTGCTTGAACCCAGGAAGCAGAGATTGTGGTG
AGCCGAGATTGTGCCATCGCACTCCAGCCTGGGTGACAGAGTCACACTCTGTCTCAAAAAAAAATAAAAAAAATTTTCAGAAAATTCTCTTTTG
TGTTATAGTTTATTTTTAATTTCAACCTGAAGGTTTATCTCCTTACAAATTTATTTAAAATAAGAGGTCTTGAGCTTCTTTCTGAGAGAAAATG
AAGATTAAAATCCACATACAAGCATTCTCCTAAAATATTTTTAGCACCAAACATCTTAATTTACATTCAAATAAATGAGAACCACCATATGCCT
TTATTTATTGCAAAGCTATGGATTATTGTACTATTGACATAAAAATCAGCTGGGAAGCTTAAGGACTTTTTCATATTGAACAGTTTGTACTTAT
GTTGTCAGACATTCTGATTTTCCTTTGAATTTTCAAGTCAGATACCATTAGGGTATGGATTAAGCCTCTACTTTTCTTTGCGTTTTTACAATAA
CTAATTTGCATGTACCTATCTTCAATTAAATTATTTTCAGTATTTTATAAAATGCCTGAGATGTTTTAGAACACAGTTTATATCATAGCACAAT
ATTCAAAATTGCTTATAAACATCAAAAGTTATGGATTAATATGAAAATTTTACCATGTCTGATAGACCACATTACATTGGGAGTGTCGGGGAGG
AAAGGGGAAACAAACTAACCAGTTAAGGAATGAAGGCAGTACTTTCCCTTCAAGTAAAGACGGGAACAGAAAACCTAGTGTAAGGAAAAAGAGA
TAGCCTGAGATCATTATCTGTCTATCAGACGTATACATACAGGTTGCTAGATCAGTATCTTTTAATTTCACATTAAGCACACTCTCTTTGAGGA
AGGAAAGTCAGTGTCTAATGATGTGAAGGCCAAAGAAAATGTCATAGTGCCCAGAAGTGGTTGCTGCTTTTAATTGGTATTTGTCATTTTAAAA
TGCTTTGTTAGCAGACAAGATTATGAAGGCTGTTGACATCAATTCTTCCCGGCCATATGCCTTTTATTGGGTTAGGAAATGTGCCATCACCTTT
CAACAAACATTTACTTATTGTGGTTCGCTAAACTCGTAAAACTATTGTTAAAATAAGGGTTAATTTAATATTTCAGACAAGGAAGAGAAATTAT
ATCCTTGTGGAATTTTAACCAAAAATTGATTTGAAATTTCTACATAAAAACATAATTTCAGTTTTTTACTTGCTTTACGTTTTATACCAAATTT
GAGAAGCCTCACAGTTTCTTTTGGTTTCTGTTTGTTGTTTTTGTTTTTAGCAGATCGCCCATACCTTCAAATATTAGAGCAACCTAAACAGGTA
AGATTAAAGGGGTGGGACTTTAAATGTTAGATTCCAGTGTCTAATATTGAGATCATAAGCACTGAAGAATAGTAAATGAGTTCTATGAAGGAAG
TAGTTTATCTGAAAGATCAACAACCTGACAAATTATGTAACAGCTTTATTCATTCACTTTACATTTCTCTTACTCATTGTTCACACTGTCCTGA
GCCTCAGTATGTAGTTCTGTAAAACTGCAGTTAATTACAGTATTAGAATTACAGTTAATTACACTATTCTGCTGTCTCTACCAGCTTAACTTAC
CAGCCACTCCCAGGAAAGGCAGAGGTGATTTCCATTCATTTTCATTTATAAGGAAATACGTATGCATACAGTTCACATCCACTGAGCGATATCC
ATCTATTATCCTTGCACATGTAGTACCTCTTAGCCTTCCTCCTTCCTTTGGTAGCCCCAGCCTTTTCTGTGCTGCCTTTGGAACATGTAACCTA
ACATACTGAAAGTACCAGTGCGAAGTAAAATGTTAGAATGAACTATTCAATTCTCTTCCCTCTGCTGTCAGCCTCTTTGGAGTCAGGAGAGGAA
CATTTTTTCAGGTGCTTCCCCTTACCACTAGAGTCTGTAGAGAATAGCTCAGAACTGAAATGGCCTCCTCACTGTATCCAACTCTGTGGATATT
TATAGGAAATTGGATGGGACCAATGCTGTGAAAGAGAAAGAAAGGCAATCTTTAATGTCTGCCTATAGGACCTTTCACTTATTAGGGAATGGAA
ATAATGGTGTAGTTCCCCACAGAATGAGGACAGTCAGTAAATCTTTGGATGGATAAACTAATCACATTTGTGTGTGACTGAGGATATGGATACC
TGTCCTCTTGCAGCTGTATTACCCAATACATTTTCTAAGTATTCTTTTCATTAGCTCAGGTGAGTGAAACACTTTTGCATTTTGTAACATTTAA
CACATGCTAACTAGGTCACAAGCTTAAATAAAGCATCCACTGCGTTGTGGTTTCCAGAATCGTGACATTATATATGTGTTATATTTGGTCAGCA
TTTGATATAAATGTCTTAATGATTGCTGAACAGGCTATCCTTATAAGGTTGAAATTAGATGAAAGTTTTTGCTGTGGGGGCAAGAATGCCACAT
GAAACTTTGGGCCATGGTTCTAGTTCTTGCAGGTGTCACATCCTGACTCCTCAGGTTGCCAGTAACACTCCATTCCTCTACCTCCATGTTCAAC
TCTTTTAAGCAGTTATTTCTCAGAGAAGGTCATCAATTCCCAGTGAAATTTTATCCCTTAAAGTTCTGTGGGCTCTGCCATCCTGGAAAATTTC
CCAGCTTTTAAGATCTAGGTAGTCTATGAAACAGAAATCAACTGAAATTTGTCTGCATATGCCAAAGTATTTTTTCCAATATCATTTTCATAAG
CATAGCACTACAATAAGAATTTTTAAATGTAATTCCTTATTATGCATCCAACAGTTATAAGGAAAAATTGGCATTTATGATTTACCTGAAGGGT
TAATGTAGTTCCAAAATTCAAAATTTAATTCATATAAAAGCTTATGTGAGTAAAACAATGTGTTTACCAAAGTGATGCTATTTTGATATCTGAA
TTCAGTGAAAGTAAGAAGGTTGTATTCAAGTCAGACTTTCTGACTGAATGGATGTAGCCTTGCCTTTGAGGTTGATGACTCATTTTGAAGCAAT
GGAGTTACTCAGATGAGTGAATGTGGATTAAAGGCAAAATTTTGATCTCAGAAATTTAGAATCAGAAATGCATCCAGACAATGCATTTGACGAC
ATCTTCCTGAAAACAGTTGTAAATTTTCATCCTCAGATTAAAAACTTCTGAGGATTTAGATACTCGTATGTAACCATGAAAAATATCTATTAAG
TATTGTCTATTTGACACCACTCCCAATAAGATATAAACACATACGTGTCTATATTTTTCCAACTGTCCCAAAGGAATTTTGTGATTAAAGATAA
TGAATTGTGTGTGTGTGGTTTTCGGGTTTTTTTTGTTTTTTTTTTTTAAGTTAGAAAAAGATTCTTTTTTCTTTGGCACCTCTTAGCCACAGAT
TTTCCCCAGCTCTGACGGAGATGAGTCATAGCATAGACTACTAATTTTTAGAACATCCAGTTCTGTTGCATATTAATCAGTGTTAATTAACTAA
TACTGTTCAAAAACTCAAGTTGTGTTTAAATTAGCAGTCAAGTAAATCTCTATTCTTATGGTTAGCTAATTCTGCAGGCTATTATACATGTGTT
GTTTTTTTCATGGTTTTAGAAATTTCATTTAATTATTTAAAAAACCAACAGTGTTTGCTTACAAAAGGCATAGGGCTGAAATTGTAAATTATTT
TTAAATATGAATTGTGTGAGAACCAAAACAAAGGAATTACCTCTGAAACTTCATCTCCAAAGGCTTCCCTTGTGTTTAGATCTGGAATCTCCCA
AGGAATTGTTTTAGGTCTCACTGTGATGAGTTGATCATAAACTTTAGACATTTGTGTGCATTAAGGAAATTTCCCAGACCGGGGAATTTACTTT
ATGCCTTACTTATCTGGTAAATGTGTTTGAGGACTGATCTTTGGAAATCAGGAAGTTTTTGGATAATATAGGAACAGTTCCAGATGCTTCCCAG
AGATTTAAATCAGTTCAAGGCACCTGGACTGCTCAGAGACTGCAAGACCCTACTTGGCATAATGAAATGTAGTATAGTCTAAGTAGTGCATACG
TTTTACATTTGTTCATGAGCTCAAAGCTGTCAAAATCTGCATGTTTGCTGGGGTTCCACAATTTAATGAGTAAGGGAGTGAACCCCCACCAGCA
GCTAGTGGGTGTGATGAGCTGATGGGCAGATCATCTAGTGCTGTATATCCACAGTTGGTCCCACAATAGAAACAAAAGGTGAGAATGTTCCTTA
AAAAATCTTAACGTATTTTCTTTTCACCAAAAAGTGAATCTGTAAAAAGAAAATCATCTGCTTAAGCGACTATGTGGTTATAAACTACTAACAC
TACCACAAATTGATTGACATCATTATAAAGTTGAATCAGGAAATACAGCCAAACCTCCCTTAATATGATGATCAGGTCATAATTATCCTTGTTT
TATTCTTTGTATACAAATATGAATAAGTTAGCTTTATAAATCATAAAAAGTTAGAACTGGAGTAGACCTTAAAGATGGTCTGGTATAATCTTCC
CTTTTGTACAAATCAAAAAATATGACACTTCTTAGGTGTTTATCAGCCCACAGTAGTTTATATAGTGAACGCTTCCAGCTAACTTCATTAATTA
ACTATATCTATAAAATTTCTGGGTTAGATAGTATTTAAGGGAACTACCTAGTTTTGGAATCATTCTGCTAGGTTAATATTCAATCTTCAGCTTG
ACCATATTAATAATCATAAAAACAAAAATCTTATCCTCTGAAATGCTGAGAGAAGCTTAACAGATGCAGGGTCTAGCACAGGGTGTGTTCTACA
ACGCTGAAACAGTATATCTAAATAAACATGTCTGTAGTCCCTGCTCAACCACCTCTAATCAGAGTAGATAAAGGAATGTCTTTAAGTAAGAGTC
AAGGAAGCATAACTTTTATTAATATGGTACAGTTCAGAGCTTGGCAGCAGCAATTTAAGACAAGGAAGCTCTGACTAGAACAAGCCGTAACATG
TTAAGTCTAAAGCCTAAACTCTTCAGCAGATTACTCTCCACACATGCATAGCATGACAGGTTCCATGGGCTTAGGTACCTGCCTTTTTAGCCAT
ATCTTAGTGTACAAATATCGATTAATACCATTTTTCGTAGTAAGATTACCGGAAAAGTGATTCTTGTTTACAGAGCCCTCTTTCAGTTTCATGT
TTTTCTTCTCTCATTTAGTAGACATAAGATTTAAAAATTTGTATGTACCTTTGTTGCCGTAATTTTTAATAAGTATTTCTCAAACTTAATTGGC
TTAACGTTCACCTTTGCAGAGAGCATTTCGTTTCCGTTATGTATGTGAAGGCCCATCCCATGGTGGACTACCTGGTGCCTCTAGTGAAAAGAAC
AAGAAGTCTTACCCTCAGGTCAAAGTAAGTTTGTGGTAGCTCTCCTTCTATTTGAATTCTGGAAATTTTGATTTCCTACGATTTCCAAGGAATT
GCTTTAAATGAGTACGGGTTGCCTTCGCTCCTAAGCTGAAGTGTTCACATGATTATTAAAATTTTTAAATAGAAATTTGTCTCCTAGCAATAGA
AGTGACAGATACTAAAACTTTGTTAACATTTCAATTTAGTAGAAATGTCTTCAGCATTAGCTAACAAACTTACTATTTCTTGATCATCTTAAAT
ATTTTTAAAAATTGGATACTTCCTGAAACTTTAGTAAGTCTTTGAAACAAATGCTTGATTTTCACTCTTTCGAGATTTAGTGAAAAACCAAACA
AGTGACTGAGTGTATGGATTATATAGTATATTATTTAAGATTATGAATTTCGATCCAGACTTTCTTATTAGCTGTGTAGCTTTGAATAGGTTTA
ACCAATACTTCTAAACTTCAGTCTCTTAATGTATAAAATACAAAAATATTAATAGTATATACTTCATAAGGTCCTTTAGAGATTAAATGAGAAA
ATGTGTACATGTTCCATAGAGAGCCTGAAAGTACCCTAGGGCCTTGGCCTGAGCAGATGTGTGGCCCTCTTGGAATCAACTGCTACGTCACATG
CTGGTGGTGTATTTGGTCATACCTTTTACCATGTTCAGGCTGTTTGTTTATTTGAGAATATTTTTCTTTAAGTTAATATGAGACTTTTAATTTC
ATAATATGCTATGTTTCATAGACTGACTGATGATAAACATAGACTGATAATCATTTTGCAGATAAACGCAGTCAACAATTGTAATTTAGTTTTT
TATAAAAGTTATTATTTAGTCAGTAATTCAAGGCTTGGACAAAATAGTTCTCACTATGTGTGTGTGCCCCTCAGGGTGTAACATACTTTTGGAC
TCAGTCTTTTTATTGGTTTGTTTTTGTAAGTAGATGAAGCTCACTAGAGCAGGCCCTCAGCAAAGTTGTCATGTAAAGAAATCTGGCCGGGCTC
GTTGACTCATGTCTGTAATCCCAGCACTTTGGGAGGCCGAGGCTAGCAGATCACAAGGTCAGGAGTTCGAGACCAGCCTGGCCAATATGGTGAA
ACCCCATCTCTACTAAAAATACAAAAATTAGCTGGGTGTGGTGGTGTACACCTGTAGTCCCAGCTAGTCAGGAGGCTGAGGCACAAGAATCGCT
TGAACCTGGGAAGCGGAGGTTGCAGTGAGCCGAGACTGTGCCACTGCACTCCATCCTGCACAACACAGCGAGACTCTGTCTCAAAAAAAAAAAA
AAAAAAAAAAGAAAGAAATCAGTCTTTGGTTTTGCCAGCCTTGCATTTTGTTTCTTCCTTTTCCTCTTATCCATCTTCTAGGCCCAGGCCTGTT
AAATTTCTTATCCTTATTCCCAAACAAAAGACAAAATGTTGTGACGTCGTTCTTTACCTTTCAAGTGAAACTCACCCATTATTTAGTATGGGTA
CTACTAGTCATAGTTGCATTTTAAGTCTCTTAGCTCCAATTGTGGAATGAAACATGTTTTTTCTATTAAAGGTAATTACGCCGGTAAATATGAT
GGCTTAAGTCCACAGAAGCAGACCACGGTCTTATGGCCTCACATCCTTTAGAACCTTGATGAGGAATGAGGAGCAGCAATAAAGAACTAATTTG
CACTTATTAAACACCAAAGACTTGCCAAGTGGTTTGTATAACATTATCTCTTTTAATCTCTTAGAAATCTCAGTAGCTAAAAAGTGCAATGCCT
GTGAACCCAAACAGACTCAGAGAATGGAATTATAATAACTGGCCTTTCTCACAAGACTAGTGGCTCTATATCCTGCAGCAGGCTATTGCTCACC
GAACCTCTCAGATATAATGTGTGCATTTGGGGTACTGGGGGCAGGGAGATAAGGGACAAATTATGGGCAGACAATGGTGTAAAAAGGCATGGTG
AATGATAACTCTTGAAAACAAGGGAGAGAGAGGCTCACTGGTCAATGGGTACTCACGTATTCCGCAAGCCAGGCCCAGCACTTACCTTTTTAAC
ACATGTGTCTGTGTGTCACATGTATAAATATTGTGCCTTTATGTATGTGTATATTTGTGAGAAGGGGATTGAAAGACACATGTTATCAGCATTG
ACACTGACTATAATGACTGAAGAATTAACTTTAACCGAAAAACTGGCAGTTTTTTCTGACTGGCTTGGCACAAAAAATATAAATTATGGTATTT
TGTTTACTTCTATTGCTCTAGTATTTTGTGACTAAGTAACAATTCTTGATACTAACAAACACATGGCATTAACTTTCTTATAAATTTATTGGCT
AAGAAAAAACTTTATACATCCATTTATTCCCAGTCTTCATACATCTTTCTCATGTAGAGGTTTCATCTACTCAAAAACATATTGCTGCCTCAGT
CACAAAATAATTTACAAATGAAACAACTTAGTGGGCATTAGATGTTCTTGTAGAATGAAAGTTTCTATATCAAGGAGTTTAGTACTTACTGGGA
AATATAGATATGTAAACGACTAATAATAAAGCAGGTTCAACTACAGTCACATTATTTCACCAAATCATTTATTTTTTATTCACTAATTCAGTAT
CCATTTATTAAGCATTTAATCAACTTGTAAGAGGAGGAGGAACTTAGAGGTTAGAGAATATGACTATACAAGCTGTCATTGTTCCCTTAGCTTG
TCATTGTTCACTGAAAATTGAAATGGTTCTGCTGTTCCTAACAGTTATAGGAAGTAATTTTATTTATCCAAGAAATTCTTAATTGGAAGGATGG
GTAGGAGAGAAAGAAACAGACAACGGCCAGGTATTTTTTTTTTCCAGAGCTCTTCTCCAAATATCCCTTTTAATAGTTATTTCTGCTCAGGATG
ACCTTTAAGTTTACAAAGGAATTTTAAATCCCCTTTTCCTCTAATGTTTAAGCAGAGGAAAAAATTGTTCTCCATGCATCTTTCTAATCAGGAA
GACCTTTAAAATTGTGCATGTCTTTTCTGTGGTACTTTACTAAGCACCTGCTCGGCGAGAGACTGTAAGGGAAACAGTGGGATAGATACATCTA
AAGTTTCTCAAACTTGGCATTATTGACATTTGGGGCTTGAAAATTCTGTGTTGTGGGAGGCTGTCTGACACGTTATAAAATATTTAGCAGCCTC
CCTGACCTTCACTCACTAGATGCTAATAGGATTCCTATAATTGTGACATCCAAAAAGTCTCCACATTGACAAATGTCCCCTGGGGGGCAAAATC
ACCCCTCTTTGAGAACCACTGATGTGGATGCAACAAAGAATCAACCTTAACCTTACCCATTTGCATACTTTTCTACTATAAAGTTGTCTCGCAT
AGATTACGTAGAATTTCCAAGTGTATTATTTTATAAACGTGCCTTTCTTGGTTATTAGTAGATATTTCTTATTTTCAAGATGGTGATCATGGTG
TCTGTATAATTTCTGTTAGAATATCAGAGTTAGTGTACCTGGATACTAAGGAGAAAGATAGAAAAGATAAAATATGTATCTTCAACATTTTGTA
TTCTGTGATGACCCTAAAAGCTAATGTGGGAGTTCTATTGACTGTTATAAACTATTACAGAAGAAAAAGTGAAAGTGGAGAGTTAGGTCCACTG
TGTGTGAACAATGGGACGGTCTATGGAATGATGCTTTCAGGTGGGCAGAGGCCTCGTGGAGGGTGGCCCCACACTCACCCCCAGAATCCAGAAG
CCATTCCCACAGGAACTTCAGAGGGAGAGCATTTACATTTACAGGAAAGAAATGATACTTCTGAGCCTACATTTGCCACATATTCATTAAGTGC
ATTTGTTCAGGCATTACAGTAAAATGCCATGGGCTGTGCTTTTCCTCAGCTACAGCCTCTCTCCCTCCAACCTCACACCCTGTCTTCCTGTCAT
GCCTTTGTGAGAGCGCCACCACTCTGCGGTCCCTGGAGAGGAACCTGCAAACGCTGATAGTTCAGTGCATACTCGGTCATATCGAGACAGCAGG
AAAGAGTCAGGGGCCAACTTGAAAGATTTGGGGCCTGGCTGTGGTTTGCAGGCTGTAGACACCAAACTATACTTTATAGAACATGTGAAAATAT
AGCCTGTATGGGTTACTCTCTTACTGATTGTCTAGATTTGATGCCTTACATTAAATCATTTTAAAACCAACTCTAGAAGTCTACTCAAATCCTG
CATTTTCTAGTGGAATAGTCTCCCATGCATAAAACCCTCTTTCGCATAGGTGTAGGCTACCTTACAGACCCTCTTACCATGTTGAGATTATTTC
TAGAACTCAGGCTACTGAACCATCTGGTGCTCTGCCTGTGTCTTTCACATAGCTAAGAAGAAAATTTCCATTTAGCTTTCTGATATCTTGCTGT
GCAAGTCTTCATGCTCTTTTTTTAAAAAAAATTTTAATTATAAATTCTTTCAACCATATACAAAGTTTAAAAATTCACATAACAGACACTATGT
ACTACAACACAAATTTAATAGGTATTAGCATCTTACCTTATTTGCTTTTGATAGCTCTCTCTCTCTCTCTCTGTCCTCTCTCTCTCTCAAATAA
AATAGTTACAGCTAAAACCCCACTTATCCATCACTTTGTCCTCCTTCCTTCTCTTAGGTAAGCTGCTACTCTGAACTTTCACTCCCCAAGATGT
TTTTATATTTTTACTACATAAGAATGCATCTATAAATACTGGCATTTTCAAGAAAGTTAATTTTTACTAACTTCAACCCAAATTTGCCATTGTA
TAGGATGGCTCCTTTTCAGGGCAAACATAGGTTCAAACTGGACGCCATTGAAGATTAACTGGTACTCACTTTGATCCTATTGATTCCTCTTTGC
TGTCTACATAAAAATGTATTTGTCACAATGTAGTTTGTACTAACTCTTATAGCAAAAAATAATATTCAGAGAAAAATCTAGGGGAAGTAAGTAT
GCAGACTAACTCTTATATCTTGTCCTTTCATAAGAAAAATTTCCCATCATTAAACATTTAATAATCTATTATACTGCCTATAAAATTTCCCTTT
AAAGTTTTTTCCATTCTAATTTCATCACGGAGCTCCTAATAGGAGAGGCTGGCAGAAAAGCACAGGAACCTTCCTCTAGGCCAAGCTTGTCCCT
GCCTGCTTTTGACTCAGCAAGGAGTGCCAGGGAAGGCTCACTTCCCTATTTTTCCCGAAAGCCACGCGTGAAGTTTTAAAAATCAAACTGTGTT
CCCTTTCACAGAGGTGGGTGACAAGAGAAGCAGCCCCATGGGACTTCAGAAACTCTCACCAATCCTCCTCTAGAGTGACCAGAAAGTACCCCTT
CCTATCCTAACTAATGTGACACGTCCTTCTGTGTGGTCCACTCTATGCAAAACCAGAAATTGACAAGAATGACTTTGATTGTAAAGTACAAATA
GCTTACCAGTTAGTATTCTTAGTTTCTCTTTAGTTATAAATATCAAATGCAACGGACTTGAAACCCAGCTGATAGAAGTGTAACTATTTTTAAT
CAAACATTTTCTGAAAGCTGGTTCTCACATATGTTGTCTTTCCTGACTGTTTTATCTTCAGTGGAAGACGTGTTGAAATGTTTAGGTCAACAAC
AGCTTTTTTGTTGTCCCCTTCCACTACTGATTTAAAAAAAAAAAAAAAAGGCTCCACTCTTCGTGCCTTTCCTTAAAAATCCTAAATGTTAAGT
TTCTGTCGAGGTCGAATAGAGACTACCCGATCTGCTGTGGCATTTGAGGATATCACATATCCTTGCATAATCTCCCTTTCCTTTGTATAGACTT
GTCTGATTCACGTGCTTTGGATCTATTTGGAGTACTAATTTAAAATAATTTAACCTGACTCAATTACTTAAAACAACACGGGAGAAAATCACCA
ATAATCAGCTATGTGTGCGTATAGAACCTAGTTCATATCATTTATTAGGATTTCCATTGAACCCCTAAGCTGTTCACTGCGCAAGTATTTAATT
AGGCAAAATAACCATACCCCTATGTACATTATACTCTGTGTTTTGTAAACCATGAGAAAAGCTGCTTATTGAACAGAAGGGCTTTGTAACCTGG
GGTCTATGGATCCCTGTTGGTGGTTTCAGACGCTTTTTAGATTCCTAAATTTATATCCACACTTACACATGTATGTATGTCCATTTTTTCTTGG
GAGATGAGTATAACTTTTTATCATATTTTCAAAGGGAAAGATTTAACATGGTTAAGAATCATGCTATAGCTTGTATCACTTTAGCTTTTTGATA
GCAGCTATCTTTTTAAATGATCTATGTAATATTTTTTAAGATTTTTTCTTTTTTTATTCTTGCATTTTTAATCAATTTTAAATCAACTGAAATG
CTTTATGTTGCTCAACACATAGCCCAGAAAGCCTCCCTTCAGTTTTCCAGTAGGTCGAAAAAAGATGAAACTGAATTGATGGCCAAAAGAGTTG
AATGGGCTTCACTGCTTTTTCTTTCTTGCTCTTCCATACATTTGCAGATTAAGCATGAAGCTCACCATTAGTGATTGAAGCCAAAAGAGAGAAG
GCAATTCATTGAACAAATATTGATTGAGCACTGTGTGTCAGGTACTGCTCTAGACAAGCAGACAAAACTCCCTCCCATATGTAACCACAATTGA
GACTTATAGGTAATTTTTGTTGCAACTACTTCAGAAGTTTTGTATTTTGTCCTAAAACAAAATATTACTTTGTAAAATATTTTTTTAAATAGCC
CTCTAAATCCGCTGTATATATTCAAAGTCACTATTATAAACTGGAGAAAGCTACTCATTAGAGAAAACCTATCACACAGATAATAGCTGGGAAG
CTTAACCCTTAGAAATTATTACGAGAGATATACACTCCTTGAATTTAAGAACTGTCTGAATTTTAAATTTTGTTTTGTCAGTTCTCGCTTGCTG
ACCTTCCTCTATTGGCCATTCCCATTTTTCTGCCCTCCACTCTTCCTTGACACATGATTATTTCTGGGACCACTGTTCAATCCGAAACAAATCT
CTGGTGGCTTCTTTTAAACCCAGTGGTCTCAGTCTGAAGGTATAGTGGGGCAACTCTCGGCATTCCATTCTGGAGTCTTGACCTCATAAAGTCA
CATATTGGCAATTCGCCTTGATAAGGTCAAGTGAACACACAGAAGCACTAAGATCCCCACATGTGTTCCACTGCCTCCTCCACAGACAAGTTGT
TTTTCAACCCATCTGCAGGTCCAGAGGTCAACCTCCACGCTACCCTCCACTCTCAGTCTTTTCTCCCCACCCTCCAGATGGATTCCAAATAAGA
AGCCTCCACACTACCTTCACCTCCCTGGGGTCTCATGGACATGGGCTGGAAAGTGCTTGCAGACTTTCACAGACAATGCTTTTTGCCTTTAGGT
CCCCTTCTACCCAACAGAATGATTATAATAATGGCTTGATGTGCTTACTTACATCATCTCATTTAATCATCACAGTAATCCACTGATGCAGGTT
CTGTGATCCGCATTTTATAAATGAGGAAATTGAAGCCTAGAGAAATTAAGAAGCTTAACTTGGGTCTCACAGGCTTTTAGAGATGGAGCCCAAG
ATTTCAGTTCATGCCATATGGTTTAAGGGCCTGCACTCTTAACCATCGTGGAATACCATTCATTATCATAATGTTAAGGATCTGTTTCTACTTT
GCAGGAATCTTTTAAATCTATGGTTCACAATCATGGGTGGGCTTCAGAATCGTCCAAAAATGTTTCAACCACAGAGACGCCTGGGAACCACTGC
ACAATTGCTAAATCACAGTGCATCATCTAGCCAATCTTGGATTCTACAATAATTCTATGACAGTTTCTAAGAATACATGCCATTATATAATGGC
AACATATTACAACACATAATTCAATAGATATTCAATGTATACCGTGGAATAGAATATTATGCCACTGTGTTTTAAAAAGATAATTAAGAGGATA
CGGCACAACGTGGAAAACTGCTTATAACATAATATTAAGGATAAACCTCAGCTGGGCATAGTGGCTCACGCCTATAATCCCAACACTTTTGGGA
GGCCAAGGCAGGAGGATTGCTTGAGCCCAGGATTTTGAGCCCAGCATGGGCAACATAGTGAGACCCTGTCTCTATAAAAAAAATTTTTTAAAGA
AAAAAAACAGGATTTCAAATTATTCCTAATATTTGTAGTGATTACAAATATTAGCTTCTTTTTATAAAGCCTGGGTGGAAGCATATAGATGTAT
CTTTAGCTTTATTTATTCATCCATGACTTAAAGAAGATTGGTTAACACCTGACTCCACCTTGAGTAGCAGAACATTGGACTGAACGAAAAAAAA
AAATGTGAAGCTGTCTTACTTAGCTACAGTTTCCAGCATGTTTTCTCAGGAGCTCTATGGGAGACAGTGCAGTGTGGCTCTCAAACCTTACTAC
CCATAAAAATCTGCTTCAAAACCTACTTAAAATATCCATTTTATTCCATTTCCAAAGCCTGGTAGAGTAGGTAATTCTCCCACTACAATACACT
TAGTAACACTAGAAAAAAAATATAACATTTTCTTTAATGCATAGCTGAGTTTATTAGAAAGTATGAAAACTACTGGGGCGCCGTAAGACAAAAG
AAAACTTTGGGAAAACACAATAGGATATGGGTGGACTTTGGGGCCACAGCTGCCCTGGAAATATTTGCTGACCTAGATAATCAAGAAGCTTGGT
TTTTAACAGCTTCCTAGAGACAAAAGATAACGCCTTGGGCCTTTCCAATCTCACCAATTGAAACTGCAATCACCCACTTAATTCAGGACTCTCT
CAGGGCTATAGTTTCTGTTGATGGGTGATCCTCAGGCAGAGAGGTGATGAGGAAGTTGCCTGTCTCAGCCTAGGGGGAATCAGAAGATAAAATG
CCTCTCTTGAGAATCAATAACTACAAGCCCAATTCTTACAGATTCAGAGTTAAGTGTATGCTCCCTGAATAGAGTAGGAAACCCCAAGTCTAGA
AATGAATATGAAGTTGTTCCACATTGGGGATACCCTCACAGCCTTGCAGAAAACCATAAAAATCTATGTGCCTCAAAGAATCATGACCTCCTCA
TCAAAAATTAGAAAATATGTATGCAAATAAGCCACTCTGTTTAAGAACTGGCAGAACAATGAAAAGCAGAATTAAATCCTCAGAATCTTCAGAT
AATTGAATTACCAATTGTTTGATATATAAATACAAAATAAGTTTGGAATAGAAAACACACCAGGCAGAATGAAAATAAAACCAAGTCGAATTTT
TAAAATTAAATGTGCGACACTGAAAATGATTCAACATAGTTAACATGGATAATTTGAAAGAAAGGATGAGCTGGCAGGTACAAAAGAAGGAGTA
AGAGGTGTCTAGTTGAAGTCCTAGAAAGAAGTATTACAACTGCAGATTTCTGGCTCTACATTTACAGTTTCTAGGTCTGGGGTGAGGTTTAGGT
GGTTGTGGACCACACTTTTAGGTTTTAAAGCATTGGCTCTGAAGTCATTTGGTTCAGGGCTGGGCATGATGTTGGTTCCTAGTTTTGCGACTTG
CAGTGTTACTTAACTTCGCTAAGCATCAGCTTCCTTATCTATGAAACTGAAAAAATAATACCTATGTCATAGGATATATGTGTAAATCAAGTTA
TTCAGTACATGCCACACAGTAACCACTTAAAAATGACAGTTGCTTCTATTTTTATATAACCAAATACACTACTACAAATATTGAGTGGCAGCTT
TGCTGGTTCATTTATGGATTCATGGAGCTACACAAAACCTTCACCAGTCTATTAATTTATTTTTAGTACAGTCTCTCTAATCAGTATTGCTTCC
ACTGTATATTGACCCCATATCCTTTCTTCTGTCTGGGGTTAAGATAGGAGACTGAGATCAGCTGTGTCAAAAGCTTTCTTTTAGATGTAACACA
TGATTTCTTATGAAAATCTCATACGTAGGATACCAGAAAGGAATTTATTAAATTCTTGTTAGGCTCCTTAGTTCTATTACTTTTGTAGGGCAGT
GTTTTAATTTAGGATACTTATGATAGAGAGCACCCCTTCCATAATCAGGAGAAACACAAGATGGAAATAAAGATTTTATTACTTAGTGGTCCTG
GGGAGCACACAGCACTCCAGGGAAGGCCACACACCAAGATCAGGGAGCATGTGGAGAAAGAGAGAGAAAGGAACTCACGGGCCATTGCCTTTAC
TGGGTCCAGGACATTATCCAAACAGGTTTCCCACACGCAGTTTTAATAGGTGGGTTTCAAGCAAGCAGGTATGAGTTCCAGGAGGTCACAGTGT
GATGAAGAAGAAGTCACTGTGGCATATCTGCACAGTCCATGCGAGGTGTGAGCATCAGTGAAGACCAGTCAGGTAGGCTATATGTAGCTGGCCC
ATGGGCAGCCGATCACCAGGTGGCAATTGTATAATGCAGATATCTAGATTGGCCACATAGAGGAACCGGGACGAGCTGTAGTACTGGAAACTGC
GTCGACGGTCACTGAGCCCTGCTTTAGGTATGAGAAAGTCCAGCTTATATTCACAATCGATGCAAAGGCAGCATAAAATTAAAAGCATTCACTG
CCAGCTGCCATAACAAGTTACCACAAACTAGGTGGTTTAAAGCAACCGAAATTTATTCTTTCACAGTTCTGGATGCTAGAAGTCCAATATCATG
GTCTTCGCAGGGCCGTGCCCCCTTTGAAGGCTCTAGGCAATAATCCTTCCTTGCCTGTTTCTAGCTTCTTGTCGTTGCTGACAATTCATGGCAG
TTTGTGACTTGCAGCTGCATCATTCCAATTTCTGTCTCCATCTTCACATGAATACTGTCTTCTCTCTGTGTCCCTGTGTCCAAATCTCCCTCTT
CTTTCTCTTAGAGACCTGTCATTGTGTTTACGGCATCCATAACCCTATGTGACCTCATCTTAACTAATCATAATTGCAACGACCCCATTTCCAG
ATAATACAATATTCTGAGCCTCTGCCTAGATGTGAATTCTGGGGGTACACCATTCAGCCCACTACAAACATATTGTCGCATTGAGGTTATAAGC
CAGTAGCCCTACAAGTATAGGATGATGCCCATTTCAGCCTGATAATACACTCCGAAAGAGATCACATGGTAACATTGTTGCCTCAGTTAATCAT
AGAAATACTCAATTAGTATAGAAATAACAAGAAAAATGCTTTGTTATTCTCATGTCTCATTTATCGAGTTCTTACTATGTATCTGTTATTGTTA
TCCTTATACACTTCTGCACAAAAGATGGTTTTATCCCAAGTTTTAGCTTAGAAACTGAGGCTTAATGAGTAACTTGACCAAGAATGGCTTCTCT
TTCTCCATTTCCTTCCTCTCATGGTGTTGTTTTATATAGATAAAATAATATATATTATTCTATTAATATAGATTAATATATGTATTTATATATG
TACATTTTATATATGTATAAAACTTATGTTGTAAGTTTTATATACATATAATATATATTCTCTTTATATATTGAATTTATATATATATAAAACT
TAACACTATTAAGTCTTAACACTATTAAGTTTCATGGTGTTGAGTTATATAGTTCTATTAATTATATATATATGGGGGGAGGGGTGTTTCTCCA
GCCTAGACTTCCACTTTGAAATGTAGTCTCCAGGCTCAGCCACTTGTTTGACATTGCCACTTGGATGTCTGGTAGACTTAACATATTCACAACT
GAATTTCAAATCTTCCCCACCAATCTGCTCTGCCTCATACCTTCCCCTTTCCGGAATGTGGTTGCTCCATCCTTCCAGTTGTACAGGCCCCAAA
TCTTGGAGTTTTCCTTGACTCCTCTCTTCTTCTCAAATCCCACATGTAATCCATCAGGTAGTCTTATTGGCTGTGCCTTCACACTTTTTTCAGA
AACTAATTATTCTTCAGCACCTCCAATGCTCCCACCCTGGTATAAGCTACCATCCTCAGCATCTTTCTTCCACTCGATTTTACCATCCATGAAT
GATTATTTCCTAAATCAATTATCAATAATTGTTATAAATTTGTGATTTTTCTATTTCTATCCTTCCTGTCACTTTTCCTGCTTTTTTTAAAAGA
GCTTTCTTCATTCTTCTTTAAAAAAAATTAAAATTAACATCAGTATGAACTCGTGGATTCTTTTGTTATTCATTCTACTATAACCCATTCCTGT
CATTATTCATTTTGATTCTTAAATTGTCCTATTCTTGGCCAGTGGGAGTCTTTCATGCTACCTCCTATGTCTTTTTGATATGTCCCTATCTTTT
TTTTCCTAACACTTTCCTTATTTCTGAAATTATAAGCTATTCTAGGTTCACCCTATACCTTCACTACCCCAGTCCAGGCATCGTCTGTCTCTCT
AAGAAGTCCTAGTTCCTTTTACCAGGAGAAGTTTTTCTAGACTCTTTCATTAGGCAGAGCTACAAAATTGATTATTTTAAAAATCTCGAGTTCA
TACACTAATTCATAATTCTAATTCCAATCCAACACTGCAGGCTTCTTCCCTCCCTCCCCTCATCCCATACTTGTATTTGCATTTGAGATATGAA
ATGTGTATCTCATATCCCATATTTCTTCAGTAACTGAAAACCCTCATCATCAATGATAGCAGCATGCTTACTCATTTTGCTCAGTCCAATAATT
CACACACATTTGTAGAAAAATAGAATAGTTTTAGAATCCCTATACCAATATCTGCAAGCTTACTATATTCGGTTCAAAATTTCTTTGTAGTTCT
TTTGACCAAATGTATTCAACAGAGAGCATTCAGTCAGAGTAATGTGTACAAAACTTACTTGGATTAAATTTTTTCCTTCTGTGTATTTATGTTT
CCATTGAGCTATAGAGTTAAGCTATTTTTTTATTTCTATTCCATTTTAGGGGTTTTCCCATCCCTGTTGATTTAGTAATTTTATTTTTTGAATA
TCAACATTTGTCAGTTTCACATCTTTCATTTATATGAGATTAACTAGACCCTTTAGAAATCATTATAAACTCAGGTTGTTCTATTAATTTTTAA
AATAGGTGTATAAAAACCAAAGCGATCAATCAGAGCCTTGATTTAAAGTACGTTCTGAATTATTTCAGACACCCGAATCCATCTAAACCCAGAA
CATTTCATTTGTAGATTTTAAAGACTTCTTGGCCTTCCTACCTTCCTTACATTTATGTAGCATGTGTTTATCCAAGGCACACCACCTGCCAGGC
ACAGTGCTGGGCTCTGTGTTGACACACATGAATGTCATCATCTTTCTGTAGCCCCCAGGTTACATCCCACCACAGCCAAGATACAGACATCTGT
GAGAGAGTAATCAGGGTATTACTCAGGATTTGCTGCAGGAATCCCTCTTCAAAGGATATTCTAAATTGGTGTGTGTGTGTGTGTGTGCGCGCGC
GCGCGCATGTGTGTGTGTGTGTGTGTTGAGGTGGACTGGGAGAAAATATACCCTCCTTGAGATCACCTCCTGGCTAGATAGTCTCTCCCTTCTC
TGTTTGTGATAGAATCCAAGTGGAATTTGACAGGTGTACTTCTCAAACTAGAAACTAGAGAGCTGCTATCTCACTCACCGTAAGAAGAACGAAT
CACACCAAACCTACATCATTGTCTTCTATGACAGGATTTCTACAAGGGTGAGATAGTGAGATGCTCCAGATCCCTGTTTTGGTAGTTCACGGAT
TCACAGCATCTCCCATGCTGGTGATGTCAACCTGAAGAGATATTTCTAGTGATAGGTTCCAGGGATCTTTCACCGAACACTTTAATATATTTTA
TCATTGTCCTGAATACAGCCCTATAAGGTTCTTACTAAATTTGCAGGTGACATGAAGTGATAGACAACAAACAATTTGAATTCAAATTAATTTT
GAAAGCTGAGACAATCCACCCAATTCTTCAGCAATTATATTTAAAAGATATAAATACATGCAGAAAACAAATTACAAGTACAGAGTCGAAGAGA
GATGATTTAGCAGCAACGCATGCAAAGAGATCTAGGAGTTTTAGTTAATAACAGACTCAACATGAACCAGCAATGCAATGTGACTACGAAACAA
GTAATTCCTCATTAGGCTGCATTAATGGAAACACGATACATAAGACCAAGGAAGTGGTGGTCTCATTTGCACTCTGTGATGAAACGCCACACCA
GGAATACAGTGTAGTACTGCTGCCCACCACATTTGTAGAGAAATAGAAACCAACTTGAGTGTCTTCAGAGCAGAATGCTGAGAGAACTCAAACT
TCATTCAAATCACCAGTAGTTACTGAGCCTGAGAAATTTATAGCCTCCAGGATTTGGTTGTCTTCAAATATATCAATGGCTGTGATGGGCAAAA
AAGGATTAAATGTGTCCTGTTGTCACAAGAGGATGAAATAAGGAGCGGTGAATGGAAGCCCCAGGCGATAGAGGTTTCAGTTGCTGCTGACAGT
CAGGGCTGTCTGAGGATGGAATGTATTGCCTCAGGACAAAGTGAGTTCATTGTCACTGAATTGGTTCAAAAATGACCAGGAAGACTACAGGGCA
GGGAAGCTAACTTGTATTACAATAAGTAATTTTTTTCTTTTTTATTTGTATGCCCTTAGAAAGATGTTTACTTAACTTGATAAATCTGTAACAA
GGGATAATCAAGATTTGGTTATAGGACAATTACAATTTTCTGACAGATCCCCCAAAATAATAGAAATTAATAGCTTACACACTACTTTATCCAA
GCTGTAGAATGTATAACTTACTTCATATACTTTGTCTTTAGTCAGTTTATACAGGCTGAACAACATAAAGTTAAGACATAAAAGGGTTTTTTTT
GCAGGTTTAAAGTACTACCATTTTTCCAAATTACTGGCTGATCCTAGAACTTTGAGAAACTTTGGTGTTTCCCCCCTTCCTACTTTTGTTTACA
TCATAGTACGTAGATGGTACAATATTTTGGTTAACCTCAGTCTGACACTTAGATTGTGTTCTGTTTTGCCCTATTAATGTCTAGAGTGTAAAAA
GACCAGTAAAGTAGCACCAAGAAATCAAAGAAAATTTCCCAGTTCTACTCACTTGTAATTCTCTTAATTTAGTAGGGGCTTTCCACAGGAAATC
ACAATAGAATAGTTGAAATGCTCTTTATCTTATGTACTTTCCTTCTGAAGAAAGGGACAGTTAATTGGCTTTGTTCCCAACATGGTCCAAATTT
TTATACAGGGCAGCTGTATTTACTGTAGTACAAAGGAGATTCTACACCGAATGATGTCTCTAAGTATTTTCAATAATTAAGAACCTAATTGCTT
CTCTAGCCAGTGGGTTTCAGTGAAACTGATAAAATAGATTTTCTAAGCAACAAAAATACATAATGAGGGTGGTTGTATACCTTTAAGAAAAAAG
AAATCAGTGATATTCAATCCCAAGTAAATTTGGATATCACAGAGTCAGAGATATTCTTAATTATTTTAAATTAAATTTGAGTCAAAAACAATGT
GTTTAAAGACTTTAAAATCATTTTAAAAGAATGTTAGAAATTTACATTAATAATTTCTATTTTCAATGAATAAATGAGAAACCTAGAATTTTTG
TTTTGGACACACTTTGATCATATCTAATCTGTTCTACTTCCTCTAATTATTTTTATGTTATTAGGATTTTTAAAAAACCTCTTAGATTAAAATT
TCTCAGTGTATCTTTGTTTGCGTGTCTTTTTTTCTCTCTCCACTTATGTAATTGAAGCATTTCTGACATATCCTCAGAACACACAGATTCTTAT
CAGGACTTAACTACCCAGGGCAGGGCCCTCCTTGGATTTACTGAAAATAAACACTTTGGGTACTCAGTCTCAGTTTTAATGACAAAAGTTGAAA
TCTTTGCCTAACATGAAGGTTTTTCTTTGTCTCTAAATTTTGATTTTCCATGCTATCAGAGGTCCCATTGCAACAGGTAATTTGAGTGTTGATT
GTATGTTTTCTCAGTTTGTAATTGTGGTGAAATGTATTTAAAATGTTCACAATTTTAAAGTTAAGTTTACCTATAAGGTTCTTTCCAACTCAGA
AATTCTCTCATGCTAAACTTTTTTTTTGTAGTTTGAAAATGTGATCTTTGGATGTTGTCTTCTGTCTCCTATGCATATGACTAGGAATCATAGA
ATTTTAGAGCTGGAAAATGAGTCAGACATTATCTAGTGATTCTGCAAATGAACAATTAAAAGGCCCAAAGAGGTTAGGTGACTCACGCAGTATC
ATAGCAGTACCTGGTGACTAGAACTCTGCCCACCTACTGCAGCACTAAAAATTAACCCAAATGTTTTTATCTGAAAATTTCCAGACTTCAGGCT
AGGCCTGTAAATAAATAAATAAGAAGTTAAAACATATTTGAGAAAGGTAAGCTTAATATGCCACATTTGGGAAAAGATGTGCCTGACACAAATG
TATTTGGTGTAATAGAGAACATTCTTCCATTATACCGAAATGAAAATTTTGAACATTCTCAAAATTCTTGAATTTTGAGACATTTATGTTAAGC
ACAGTTTATTCAGACTCTCTATAAACTGTGCTGTCATCCCTGTTGTTTTCTCCAAGCGGAATCATACTTAAATTACAACTTTAGGGAGTTTAAT
GTTTCCTATTTTTGCACTCCTGGGATATTTTTCAGTGACTCAGAAGCATAAACTGACACTGTGAAGTTAAGGCTGTGTTCCAGTGGGAATGACA
GCATGTCTCTAGGACGCTGATGGAGAAAGCCCTCCTCATGCAGCTGTCTTGCGGAGTCTCCCTCCTGGTGGTGAATGTGAGATCACACAGGGCC
AGCAGCCAGGGCAGTACATCTCTGAGTCTCGCTTCTGGTTTTACCTACAGCCAACCCTTGGTGATTGTTTGCCTCCTTGGCCAAGCCCCCACAC
AATAGCAAAACTGTGTCTCAAGGTCGAATTCTATTAATACCTGACAGCTCAATTTATAAACATTTAAGATGTTTTACCATCTCTGCAGCCAGTC
TCTCTCTAAATGAAAATTGCTTCTCGAAGTAGAAAAGCATAAGGGAGTACAACGGCTTCAAAAAAAAATCCTTTTAGGAATAAGAATATTGCCA
GGGTTAGGTGTTCCTAACTGGTTTCAAGCAGAGGAGTTAGTAAAATTCCAAAAGTTAATCAGCTTCATGTACTCAACTTTTGCTAGGACTTTGC
AGAAACTCCTCAGGGATCCCTCAGAGTTAGATTAGGGAGATGAACCCTGCCAAAGGACAGTAACCATTCAACAGCTGGATAGCCTGTTGGGCCC
CACTGTCCTCAGCCTTTCCTAATGTTGCCTGGGAACACCACTTTTCTGCTCCCTCCTTTTTCAGTGTAGCCTCGACACAATTATTTATTTATTC
ACTAGCTCTTTTAACAAATATTGATTAAGTAGGTACTGGTTCTGTTCTAGGGGTTGGAAAAACAGAGAAAATTAAATAGTGCCTTTGTCCTCAG
GAAACAATCCATTTAATAGAGAGACGAAGATATGCCCTTAAATACCTGAATCACCAGCATAACCAGCACCTGCGGCTGCCATGTGGGAATTGTT
GAACAAGATAATTTTTGAATCAAAGACTAAATGACCTAATTGCTCTAATGTAAAGATAAATTTCTAAGAGTCGGCTAGTTGGGATGATTATAGA
AGGATTCAAGGGAGAGATGTATGAAACCATAGATCTAACGAGAGAAAACAGCACAGCAGAAAAAGCAGCAAAGGCTCAAAAGTAGAAATCTCTG
AGATCGAATGGAGAATAGTAAATAATTTGCTGCAATGAAGAGCACGTGGAAGGCAAGGGTGGTAAGAAGGCTGGAGATAGTAGATAAGGCCAAA
TCATGCAAGGATTTAAACTGTATGTTTTTAGAAAATGGGTAAGCTATAGAAATTTTATATACCAATGAGAAATTATCAGCAGCATTGTGCTTTA
GGACTACAAATCTGGTAGCAATCTGCTAACTGATATTGGAAGGGAAAGGCCCAGAGACACTAGTTGGAAGAATTGCAGTGATTCTTCATGAGCT
AAACTAAGACTTCAAGCTAAGATAGATAAACTAAGGAATGACAATGGAAGAAAGGGACAAACTGAGATTTTTGGGGTCGTACTCAGCTGAACTT
TGGCAACTGGTTAAACATAAAAGACAAGAGACAGAGAGGAATCCAAGATGAAAAGACGATTTTGCTGCACTTTTTTAGAAGGCAGCATTTGTAT
TTCCATTTCCAGGAGGTGGAGTAGCTTTGAGAAGAAAGGTGATTAGTTCAGTTTTGCGCACATTGAGCTGTAACGGTATTATGGACAGGAAGAT
ACCCTACAAACAGCTGGAAATTTTGCACATGCTTAAAAAGAAGATGAGAACATGAGATTTCAATTTAGCCATCATCTGCATGGAGTAGACAATT
CAATCCAGGAGACTTGATAAGAATGTCAAGGGAGTGATCACGATAAACGTGAGAAATGGACTGAGCATCGATCTCTGCTGAAACATCTCTTTGA
GCAGGAAGAAGAGTGCCTTAGCAAAAGCAGCCAGTGAAATGGGGAGTTGGCGGAGACTTTCAAGGAGGGAATGGTAACATATCCCATTCCCAAA
GAGGTCAAGTAGGGTTGAGGAATAATAAATGGCTAGTGGGTTTGGAAACATGGAATCTGTCATGACCTTTACACACTTTTTCATTTGATGGATC
GGGGGATTACTTACATTTGCAAAGGGCCTCAGATTAAGTGGTAATGGTTCAGCATTAGTAATTAGAGATTTTCTTATGTACTTTGTAGGGACAA
ATAGAATGGCATTCAAACAGGTTTAAGTTCCTCTTAAGAACCTTTCTCATTGGATTCTACAGAAGAGCAAATGAAAGAATCGTTCACTATTTCT
TGGTATCATGCCCAGCCCATAGGTTTCAATTAATGTTAACCTTTACAGTTGTCATCATCATTATCCAGAAGGGGCTTTTTTTTAGAATAAGGAA
GAAAAGCAGTCCTCATCTGTAGACAAGGGAAAAAATCCTTTGGAAGAGAGAAAGAGAAGGGTAATTCAAGAAGATCAGGAATAAACATTAAGGG
TAAAAACAGAATGGTTAGCCCTGGAAAAGGGAACAAACATGTTTCTCTGAATTAGGAGAGGATATGTGAAGTAACAGCGATGTTTTGAGCCTCA
GTAACAGTAAGGAGAAGAGAGAGTGAAGGAACTTGCACGCAGTGACTTCTACCTTCTCATTAAAGTCAAGAGAGAATCTCAACTGCAGATATCT
GCTTAACGTAGGAAGACAGAAATAAGAATTGCTGTCATTTATTTTGTGGTTTTGATATAGCAAAAACTGTACTAGACACTTTACCTCCTTTAAT
CCTCAGAAAACTTAGAGATTATATATTTGTATTCACATTCTACAAATGAGAAAGCAAGTAAAAAAGTTAAGTAACTCTCCCAGAGTCCCCCAGA
TGGTCATCGGAGCCCACATTCAGAGCCAGCTATGTCTTACTCCAAAGCCTGTGCTCTTTATTGAAACATACTCCACTAGTTTGAGATCTTGGAG
GTGTACCCCTGCACTACTATCAGGACTACCCCTGATAAAGTCCTGTGGGGTAGTTGTTCCTATCCATCACCACAGTGAATATCAAAGAAGGGCC
CTCTCATTTGAGGAATAACAGACAGTGAAATTGAGATGGACCAGAAATCCAAATCCTGGATCCCTGAGGGAAACAACAAAATCAAGCATAGAGG
AAATATCTGAGTCTGGTGCCAAGACTCATCAGTACAAGAGAGAATGCCTAATGGGCAATAGCAGTTAGGGTGGGAAAAGAGAAGTACAAGGGGG
TGTCTTGGAATTTGAGGGAAGAACTGCTGCCTTAGTTTTCGAAGGATCATTATTCGAGAGTTGCCCTCCCTTCACTTTCTCACAGCTGGCAGAA
TTCCTACCTCTTAGTGGTTAGATTCCTTTTTACCAGATACTGTAGGCCCACTGCAGCAACCACAGCCCCCAGCCAGAAACCCTGCCTCCAATTT
AGACCAGTGTTCTGCTCTGGTGTGTAATAATCATATTCTAATTTAAATAGCCTATTGAGATGCTACATTGATATCTAGCCTTTTTTTTTTTTTT
TTTTACTTTTAAGTTTTATGTGCAGGATGTGCAGGTTTGTTACATACGTAGACCTGTCATGGCGGTTTCTTGTACTGATTATTTCATCACTCAG
GTATTAAGCCTAATATCTGTTAGTTCTTTTTCCTGGTCCTCTCTGTCCTCCCACCCTCCACCCTTTGATAGGCCCCAGTGTGTGTTGTTTCCCT
CTATGTGGCAATGTGTTCTCATCATTTAGCTCCCACTTATAAGTGACAATATGCAGTATCTGTAATAATGGGTTTTCTGTTCTCGTGTTAGTTT
GCTAAGGATAATGACCTCCAGCTCCATCCATGTCCCTGCAAAGGACATGATCTTGTTCTTTTTCATTTCTCCATAGTATTCAATTGCCCATTTT
TTAAAAGACAAAATAACAAAAAAAAGAGTACAGTTATCTCCACTCCTCACCCACTACCTGCCACCTAACCCAACCCTTTATATTTTAGTAATTG
CCTTCCATAAATAACATTTTATCCTGTCTTAATTATGCATTCATTAGTGTATTAATTATTGAATGCCCACAATATGACAAGCATTATTTCAAGC
ACTGAGGATACAGCAGAGAATAAATCAATATCCTCACTGTTTTTATTCCATTTTTTTAACAGACAGGGTCTCACCCTGTTGCCCAGGCTGGAGT
GCAGTGGTGCGATCATAGCCCAATGCCACCTTGAACTTCTGGGCTCAAAGGACACTCCTACCTCAGTCTCCCAAGTAGCCGGAACTACAGGTGC
ACACTACCACACCCAGCTTCCCCACTCTGAGCCTCCATCCTAATGGAGAAGAGAGGCAGTAATCAAAATACATGAAATGTGTAGTTATTAGATG
GTTATTAATGTATAGACAAAAATTAAGCAGAGAAAGGAGCAAGGGATTAGGGGGTCAAAGTTTTGGTAGGGGTAGAGTGGTCAGTGAAGGCCAC
CTTGAGAGATGGCAATTATGTAAAGCCTGAAAGCAGGTAAGAAATATACAGGTATACAGATGTCCCTGGAAAGAGTGTTCTAGGCAGAAGAAAT
AGAAAGTGCAAAAGCTAAGATTGCTCACCTGGCAAGTTCAAGAACAGTGAGAAACTGATGTGGCTGCATAGGAGTAAGAAGAAAAAGGACGGAG
AGGACATGAGATGGTCAGATAGCCTAGGGCCTTGTAAGCCATTATAACGATTTTGTCTTTTAATATAGAGAAATATTAAAGGTTTTTGTTTTAA
CTTTTTAACATAGAAAAATGTAATCATATACAAACACAGAATAGTATAATCAGCCCCCTTGCACTAATCACTCAGCTTTAACAGCTTTCACTTT
GGGAGGATTTTGAGTGAAGGAGTGATAAGACATGGCATGGCATGACATGGCTTGAACAGGATCACTCAGGAAAATAAGCCATGGGGAGACAGGA
CAAGCAGAGGAAACACAGGAGCAAAAGCTTAGGAGACAGATGATGGTGGCTGGAAACCAAGGGAGTAGCAGTGAAGGTCGTGAGAAGTGATCAG
ATAGAGGATATTTTTTTGAAGATTTGAATTAATATTATATATATTATACTTTAAAGAAGAATTTGAAAATGCATCTAGTGTAGTAAACATGGTA
AGAAATACAGAAAAGACCCCAGGCACAGTGGCTCATGCCAGTAATCCTAGCACTTTAGGAGGCTTAGGTGGGAAGCTCCCTTGAGGCCATGAGT
TCAAGGCCAGCCCCGGCAACATAGTGAGACTCTGTCTATATGGAAAAAAAAATTAACCAGGTGTGGTGGCATGCACCTCTAGTCCTAGCTACCT
AGGAGGCTGAGGCAGGAAGATTGCCTGAGCCACGAGGTTGAGTCTTCAGTCAGCCATGATCATACCACTGTACTCCAGCCTGAATGGCACAGCA
AGACCCTGTCCCTTTAAAAAAAAAAAAAAAGAAAGAAAGAAAAAGAAATATAAGAAATGGCACTAGAATATCAGTATTATATACATTATTTTCT
GTTTAAAAATCCACACAACTGGCAAAAAAAAGTGACATACATGAACATTTATAAGACAGTGCATTTATAAATTACAAACTCTCTGCTCCTCCCA
TATTATTTTGCAGTGATGAATGATTACGCCATCCAAACGTAAAGATTTATTTGTAATTTGCTAATGATGGAATGACACCTTTTGAGGTACAAGT
TCCCAGCAATTCTTGGAATTAGCAATAGTGTGTTACTTCTTTCTCGAAAGAAGCATGATTTGTAAGCAGCTATATCTGATGTAGTAAAACTGAA
AGTTAAAATAAAAAAGGAAACCGTGGGTCAAAGTATGAGGGAAAAGATAAAACTTTCAGAGCAAAGTTATACATAGAGATTTTATTCATCGTCT
CTGTGTAACTCACGAGAACTTCTTAGCTTATTTATTATGTCAAATGGCAGTTTGCTCTTCTAATCCCAGCCTGATATCATGAGCCATATATGCT
GTTGGTCATTCAAAAAGGGCACTAAACAAGGTGAAAGAATGTCAGTGAATCAATGCAACTCCATGAGTACTACCAGGAAAAGAAAAACAAAAAA
ACATGCCATTAATAGTGGGTCAAAGAGGATTTTTTTCATGCCATCCCCTACCTTACTCAGAATACCACATTTATATTCAGAAGACTCATAATGA
ATTCCAGAGTCCTCTGTGTCTTGGCTGTATGACTTTGGGTTATGTCACTTTGTTAATCTGTTTGAACTTCTGTTTTCTTATCTACAAAATAGGG
GTAATAGAGTCAACTCTGCCTCCCTCACAGGGACTTTCGGAGGCCCAAGGGTCTTTACAAATAATAGAGAAGGAAAGAAACTGGCACTTTAATA
GTACCTACCATGTACCAGGGACTGCGTAGTCCCCACGGCAGCCCTCTGAAGTCATGTGATTGGCCCATTGTGTAGATCTAGAGCTTAAGACTCA
GAGAGGTTATTTAATCTGTGTAAGGCCACAGAGCTAGGAGGGGGAAAAGCTGGGATTGGATCACTGACTAGATCAACTTGAAATCTCCTGCCTG
ACAGAGAAAGAAGAGACTAACTTTACTAAGCATCTTTTTATGCCACAAACTGTGTTGGACATAATTCTGTACTATCTTATTTGGTAAAGGTCAT
TCTGTCCTTCCTGGTTGCCCTGGTTGCTCAACAAGTAATAAACAATAAACCCTGACTGTTTTTCATCCCTGAGAGAAGAAATATTATTAAAATA
GCGTTATTCTCTTTCTTTCTTTCCTTTTTTTTTTTTTTGTCTGTGTCTGTGTGTGCGCCTGATAATCTGACTTGCAACCTCACAGAAAAGAATG
AATGCATGGTTTCATGTCTGTGGCATGACTCTGTGAAGGAGCCTTCCAAGGGGACAGATACACCTCTACCACTTCTTATAGCCATTCCTGAGAC
CTCTCTTCTCACAACACATACCAGCAAAGCCAAACAAACAAGGTGTTTAGGAATAACTGTCATCTATCAACACTCTCTAGGCAAAGTAGAACTA
TGAAACACTGACGAGACTGCACACACACAGAAGAAAAAATGATCATGTCACATGGTAGCACAGAGCAATGGGAAATGTGAATAGTTTGTTGAGG
TGGACAGACCCAAGCTCAAGGATTGCCTGATCGCTCCATTTATAGGAACCATGGCAACCTACCTTATCTTACTGAGCCTCAGTTCCTCCAGCTG
TGAAATGGGGTACAACCTCTGACCCCACAGGGCTGTTGTGGGGATTATCTGAGTTAGCATGTTCCATAAAACACAGTAAGTTAGAAAGTATAGC
TGTATAGGCTCTGGCAGTTTAACAAGCAAGAGAAATTTGACAGTAATCATTTAGTTACTGAACCCTGTCTTCATATTGCGCTAGGTTGCATATG
GTTTTCCTGCCTGTCCCTTCCAATCACATTTCAGAAAAAAGGAGGCAGTAGTGGAGCCTTGTGGGCTAAAGTGGTGAGGAGGGTGTCATGGAAG
ATTCAGGTTCACCTCTTCCTCTTGAAGAATGGGTGCTTCTCAATAGGGAAGTGGACGAACAACCTGGGAGTTCTCAGCTTGAAGAAAATCCTGA
GACGTGATAATGGGTAGGTCCAAGGAGGTCCTGAGTAGGACTCTGAGCACAGAGCCTTTCTTTGCATGTGCTGTTTTTCCTGCCAGCAAGACCC
TCTAAACCCTTTGGCCTCACCCACCCCCTTGCCTCCTTATACTCCTAGCCTTCTTTCAGATCTCCACCCTCAGTACTCCCTCAGGAAGCTTTCC
CTGACCTCCTGACCCAGTCAAGTGCTTCTTATATATCTCCTTTGTAAGCTCTTCTCAGTTACACTTTTACATTTATTTGTCAGATTATTTGATT
AGTGTCAGTCCCACTCTTTAATTCTCCACAAGAACAGAGATTGTGTCTGTTCTGCATTCACCATGCATCGCTTCATCCCCTAGTTTACTGCAGT
GTGTGGAACATAGGACGGGCTTTAAATACTCGCTGAATAAATAGAGAAATGTTCGTTATTAGTGAGGTATTACCTAGGTGGGACAGAGGATTTA
TATTTGGGATTGCTAATCCCATTAAGTGCCAAGCACTGCATTAACTCATTTAATCCTAAAAAAAAGTCCTATGAAATCAAGTGTATTATTCCTT
TTAACTTTAAAGACAGAGAAACTCAAATCCAGAGATGTTAACCATCTTTCCCCGTTGACACAGCTAAGTGGTAACAGTCGGTCTTGAGCCGGGC
AGCCTGGCTTCACACTTCCTAGACTTTACCACATTGTTCTGCTGCTTAGTGCAGTAGAGGACAAAGTGAGGTGCACACGTCAAGTCAGGACAGA
TTTCCGTTTGAAAATTGTGTATTTTCCCCTCCTCTTCTTCATTACCATTGTCATCATCATCATCATCAATCACTTCACAAGCAAAGCTAAGCAG
AAAAGAACTACTTCATGTATCACCTCTTACATTCATGTTAACTCTACATTCTGCCAAAGTGATTTGGCTGAATTTACCCAAAGAGCAAAGTGGA
CATTTCAAATCCTGATTCCTAGAAAATTATAGACTACATCCTGTAACCACCTTTGAAGAACCAGCATGTACTTTACCCCAACGTGACAAAATTG
TTTTAAATAAAAAGAGGTAATTTGACTAAGATTGGTATGATCTGAGACCCATGGTTTCTACATCTTATTCAAAAAGCAGATCCAGCATGTGGTC
ATCAGTAGGTATTGAGTCATAAAGCTCCTGGAAGAAAATCTTAAGTTTCATCAAGAATGCCAAGAAGTCCTGGGTGTCTCCAAGAATGATAGCT
GTTACTATAATAACATAATTACTACTGGAAAATTAGCAAAGCCCAATAAAATATGGCATGCAAAAAAATATTTACCCTGGAAATAGAAGTGACC
TAAATCAGTCGTTTATTCCAGCCACCTGCTTTTAGATAGTACTTAAAAAAAAAACTTTCAGGATGGTCATATTGTTTAAAGTTCTCTAGGTGGA
AGAGTACTTTACAGTAATAAATTAATAAATAAGACAAGTTAATGCTTTTAAACTACTATTATTTGTTGAGCATTGTTGTAAAGGCTTTACATGC
ATTAATGTATTTAGTCCTCACAACAACCCTTTGAGATACTATTATCATCATCTCCCTTTTACAGATGGGGAAACTGAGCAGTGCCCAAAGTCAC
ACAACCAGGAAATGGGGAACTATCTTCAAGCCCAGCTGGTTTGCGCCTAAAGTCCATGCTCTTACAGTGTCCCTGGGTAGCTGAGTACTCAGTT
TTGTCACCTTTGTCTCCTTCACTTTAACTGAAAGCTTAACTCTTTTTTTTTTTTTTTTTTTTTAAACGGAGTCTTGCTCTGTCACCCAGGCTGG
AGGGGCAGTGTCTTGATCTTGGCTCACTGCAACCTCCACCTCCTGGGTTCAAGTGATTCTCCTGCCGCCTGCCGAATAGCTGGGATTACAGGTG
CACACCACCATGCCCGCCTAATATTTTGTATTTTTAGTAAAGACACGCCTTCACCATGTTGGCCAGGCTGGTCTCAAACTCCTGACCTCAGGTG
ATCTACCCGTCTCGGCCTCCCAAAGTCCTCCGATTACAGGCATGAGCCACCACGCCCGGCCCGAAAGCTTAACTCTTAATAAGAGCCTGCAATG
TTCAGACGCTTTGAAAAAAACCTAACAGACACAGTTGCTCTCTATAAACTTAATTTTCAAACGAACCACTATTCAAAGAAGTAAAAGGGAGACT
GAATAAAAAGAACTAACTTCTCCCTAGAGCAAATGAATTTCTCAATCGATTGCAAACAGTTCTCCGTCATCATGACATCCTATGAAGTATGGTC
TCCCCCATGTTAGAGTGCCAGTGTCTTCATGTACCATATAACTTGCAAATAGTATCACTGTCTTCTCACACTTCAGCTTTCTTCTTTCATCAGT
ATTTTGGAAAGTCCACAAAACCCAGGGGGCTCCCTGTCTGTTGCCCTGTGGTCTCTGTCAAAACCAGATGATGCCATGCTGACTTGCACTCTTT
CAAATGGATCCAACACCACATGAGTCACTGGCAGCATGTTCAGCTCCTTTACCTCAGGAATTACCCAGTTCCTCATTCTTGCCACCCTTCAGCC
GTGAAGTAGTAATATGAAAAATACATGAGTACTTTTTGAGGATACCTCGGACCTCTTTCCTTCAGTACCAGAGACTAATTGCCTCATAATGGTA
ACATTGAAGAGCCCAGTTATCTGACGTGATTGGGCTTTAATTATGGATTCCTTCTGGTGTCCTATTAAATTGGAAAGCCTAGCTGATTTCACCC
AAATGAAGCATTACCTCATGCCTTGCTCTCTTGCATTCCTGTGCTGGTGTCACTTAAAAGTGTAATTACATGGTTTATTTCGTGTTCTTTCACT
CTTGATTTATTGCTGAAGAAGCCCCTGTGGGATATATCATCATAACAAAGACTGATTTCAGATGTCAACACCAAGAAACACAACTGGCATCAGC
AATCTATAGAGAGTATCTAGGGACAGCAGTCAGCTTTGGAGAAAACCCAGAACTTCTCATTCCCCATCCCACATTTAAGGGCAAAGACTTTCTC
CTTGTTAAAGGGAAAGCACTGAGTTCATAATTAGCTTTATACCCAGAGTTTTTTTTTTTTAAATGTTATACCTGTTTAAGAAATTTTTTCTGCT
TTGAGGTGGTCAAGATCAAATTTAATTAGCAACCCTCTTCGTGATTGGCTTGCTCTGGGGACCCTGCTCTTTGAGAACGAGGTTAGAAGCTTTA
GAAGCAAACCTTTGCAGCAGTTCTAGCCAAGTGCATGCATACAGGCCTTGAGGTGGAGCTGCACAGTTGACTGTAGCGGAGTCTGTCCTTTGTT
GACGAGTGAAACTTCTCTCCCTGCAAACCCTCTGGGGAATAATGCCAACTCTGATCAGCTTTTCGACTCTTGGATGAAATATTGGGTTGGGAAA
AAGTCCCTACTAAGAAAATTGTACCAAAACCCTGCTTCAATAAAATATGAATCCATTTAATGAAGTTTATAGTAGAAGTAGGCTAGTCGAAAAG
GATTCTGTCTCTTTCCAAGTATTATTGGCAGGTTTAGGGTCTTCCTCAAGTTTATATCATTAGGACTTCCATCAAAGAGTTATCTTTAGGTGTG
ATTCCCATCCTCCCTCCCTCTCTCCCTCCCTCCCTCCGTCCCATCGTTCCTTCCTCCCTTTCCTCCTTCCTTTTCTACCTTTCTTTTTCTCTCT
GTCTCTCTTTCTTTCAAGCTGTGGGCTCCTTGTACAGATTTATGCATTTTGAATTACTGCATAGTTGAGAGATTTGGGGCTTAGGTTATTCAGT
TCTTCAAAAAGTACAGTATGAGCTAAGCAGATACGGTGACCAACTAATCTAGGTTTTGGCTCCAAAAACCCTGTGTCCCAGAAAACCCCTCAGT
CCTAGGCAAACCAGGATAGTAGTTCATCTTGTGAGGAGATTATAGTGCATTTCCAAGTATTTAGCTGAATTTATCACAGGGCTCTGACTTGATA
GGATTTAAGAGGGTTAAAATAAGCTCTTTTCTGTTATGTTAGTGTGACTTTAGTCTTGGCACTTTCTCAAGGAAGGAATCCAGTCTACTACAGG
GGCTAGGTGACTAAGGAGAAGAAAGAGGATATCTAAGTGTTTTTTGCTAAAAATGTCATTATCTCCATGCTATTTCCTTTTTATTCCTATTGTC
ATCATTTTAAAGACATACCAATAAGAAATAAATTACTTTAAATAGAAAACTCTAATTTTATCAAGCAACACCAAACAGGGGTGAAATCTAAGGA
TTTGTACTTAAGGAGTACACTTTTTTGAATTGAAGAAACTTACTATGACCTGAGCATATGTAACTCAATTATCAGAAGTCAGAGAGGGCTACTG
AAGGCTCAGGTTTTCTAAGAGATGACTGGACGCCAAGTCCTCAGGCTTGAGTGTTTATAGCCAAACTTCAGAACCAGGTCCCTGGCAAGATTCT
CCTGTCTAATCACATGGCACAGAGTTAGGATGGACTGTTTCTGCTTACCTGCCTTCTTCCTTTGATTTCCATCAACCACCAGTTTCTTTCTCAT
CAATCATTTACCCAGCTTTTGTGACCAAGAATTTTGATGAGGATGCAGAGGAAGGCATGTTCTTGGTTAATAGGAAAGGAGGCTTCATGACAAC
ACAGCATCCTCTAAGATGTGTTGCTCCAACTTTCACAATACATATTGTGTTTTAAGTGGCCTGCTTCTTGGAGTGTATCTTCCCACTACACAAA
ACAACCACCATTAACATTTTCATTTACTTCCACCCTGTCATTTGTTCATCACATACTTTTAAAAACAGGGCTGTAATCATGTATATACATATAT
GAATAACTTTATATTGTACTTTTTATACTTACTGGCTTATCATAAACATGTTTCCATGTTGCTCCAGAGTCAGATATGCTAACTTTTCGAATGT
GCTTCTTATATAGATCTGCAACTATGTGGGACCAGCAAAGGTTATTGTTCAGTTGGTCACAAATGGAAAAAATATCCACCTGCATGCCCACAGC
CTGGTGCCAAAACACTGTGAGGATGGGATCTGCACTGTAACTCCTGGACCCAAGGACATGGTGGTCGGCTAAGTAGGGGTATATGATGCTGTGG
AAGGTAGGAACAGATAGAATATGGGATGCACGAACTTAGAATGAACAGGCCCCTTTCATTAGGGACCTTTCTGAGACCCTCAGATGACCTCAAA
AAACTGTGTAAACTTGTGCTTTCATTTGGACTCCAGCAAGTAAGGTGACCACATCTCTGGATTTGCTTGAGACAGCCCAGTTTGCCCCTGACCT
AATTGTTTATAGCACCCTCTTCTACTCTTAAAGGTGGCCCAATTTCGATGATCACTTATATGTGGTCACTTGACCTACAAGGTATTTGTGCCAC
TATTAATTTTTTTTCTCCTAGAAATAAAAATAAGGACAAATTTAAAGAAACATAACTATAGCAGAAATCATCACTTTATGTATATTTTTACATT
CTTGATGATTTCTTCAGTGGATTTATGGTGTGAATATGTAGGGCTTTAAGTGAATGCATAAATATCATTTCACCTCTCTGCTGCTTAGAAAGTA
ATGCACAGATAAGAAGCCAAGGAATGCAAATTTATTTTGGCATTCATTAGGCTTGAAATGTAAATAAAAAATGACGTATTCATTTAAGGGTTGA
TCAAATAAAACAAGCAATTTTTAATGAATGAAGTTTTATAGAAGTAATTTCAATCCAAAAGAATTAAGCTTTTAAATAAGACATATTTATTGTG
CTAAAAAACTTGTATTTTCATCTTCCTAATACACTAAAATCGCACACAGAATAATAGTGTAGCACACAATACAAACTGATATGTGCAATCAATA
ATTCTAAAAATTGTTGTTTTAAGTCTTTCTTTGGAAGTCCTTAAATGCAAAACAAGAAATTTTTAATTTTGTTTAATTTTTAAAAACATTTTTC
AGGGTTTTCTAATCCTGGTTTAAATAGTTTATGCTGAAATCCCAGTTAATCTGGCTTATTCACTCAAAAGAAAGGAATTGTTTAAATCAACTAT
AATTAAGCTGATGGTTGCATTTCAGGCTTTGAATTCCGATCAAGTTTCAAATTGGTTATTATTTTGGAGGTTGTGCTTTCATGTTCCTAGGTAT
TTCTTTGTTTGACTGTGAAAATATTTAAGTGTGAAAAACCTAGAGAGAGAGCTTTCTTTTAAGTCACTAAAATCGGATTCTTTTGATGTTAGAA
AAAAAAAAGACTCTAGATTCAAGGAAGAAGCTTAAATTGATTTGAAAAATTAAAGTGTGCTTTATATGAACTTGTTTCAACATCCTCTTGAAAG
ACTGGCGTCTCTCCTGTTGTATGTCACAGTTTGTTCATCATGTGTTGTGGAATACCCTTCCAGTTCCTGATTGACCTAGGAGAGCTTTGAAATG
CAGGCTAATTATCACAGTTTTAGACTGATTGATCACTTAGTGTTTCTGCAGATTTATTCCAGTAGCGTTCTGTCAGTTTGAAGTGTTTATTTTC
TTTGGTCTTGTTTAATCCCTATTATAATCATATCCTTTAGAGGAAATTTTAGATGAAGTTTTCTACTAGTATTTATTATTATAAAACCTCCAGT
ATGGTGTCTATAATTTGGTGTTTCTGTGGTTTGCTTTAGTAATCTTTCAGGATTTTAGAAAGAACTAATGGTTATTTAAATTTGAGACCTTAAA
GCGGGAATAGAACTAAATTGTTTCTAAGGAAAGTGAGGGATTCTTTCACTCTTGTAGTGCAGTGGGGTGTGGCCTCAGTAGTTGGGAGAACTCA
TCAGTCATTCACTCATTCAACAAACATTTTGAGCATCTCAGCTCCTGAATTTGATCATTATGATGTCTGGTTGTAGAGCAGTGAGATTTTCTAA
CCCTATATAATTTCAGTGAGAAATTTAATCAACATGCCACTTGAGAGTTACAATAGACTCAGTAGTCTTAACGTTTTTAGTGTTTCCTATGAAA
AGTCAGTACTCACTTTCTAATTTGATTTTTAGCTGAGAAATTTTTGCTTACAATGTTTCCCAAAATACACTATTTCTTTCATAATTCATTTGTT
TCTATCTCTCTCTATATATACATTTTTAAGATTTTCAATGTCAAAACTTATTGCATTTATGTAGAAAGAACATCGTGCTAGCATTCTGGAAACC
AGTATTTTATTCCCAGTACTGTTGCTCTCAACACAGGACTGGGGGCAAGTTACATGACTTCTTGCGGACCTTAATGTCTTCCCTCATAAAATGA
GAGAGTTTCACTACAGAATCTTATCTTCAGGCTGTTTACATCTCCAGAATTCTCGACTGAACTAACATTACGTAATATTATGAAATAACATGAG
CAAACTTGCCACACACATATTTATGGTATGACATTGATTATTAGTATTTAGTTCTGACTTGTAGATAAGAGTATCACATAGAGAAACTTAGGAA
GATAGCCAAAATTATGTCTTAGACTTGTCAATACTAGAAATGAAGCTAAGATAACTTGTTTTCAATAATACAGATTACCCAAAGAGTATAGATT
CACAAGCAAGACACTAATCCAACATACCAAAAAAGATATCCAAAAAATGGCAATGAGAAGACAAATAACTTTTAAATTGTCAAAACCAAAAGAA
TTACTTTCTTTGGAGTGTTTATCTTCATCTTTCCCTCAGTATGAATTCTTAATAAACTTAGTTTCTACTTTCAAAAGGATTTTATCTATGTTAT
AAATATTCATGTTTTAAATAGCCAAGTCTTGATTCTCGTTTGATTGCTGTAATGTAGGGGATTTGTCATTTTAATATTTTTTAAGTTAGCTTTA
ATTTTGTCTTTTTTAAAATTCATTTATATAATTAGCCTTTACTGCCTTTCTTGGTAAAAAATAATAATAATAATCTTAGTTTGGTGTTTTATTT
TAAACCCTGAATATCAACAATTTGTTTCAACATATTTTAGTTCGTTGACATTTTTATTCCATGGAAAATATCTGTGTTCCTTGACTTTGCTTTA
GGCCAGTTCACTTACCAGTAATCACCACACTCTCTCTGCTTTCTGGCAGTTTAGAGACATGGAATTTTTTTCCAATTAATACATATAGCATATA
CCAACTGGTAAGCACTAGGAGTAGATATATAAATATCAAAATACAGAGGAGAAACAAGGCCTTCTTCAGTGTCTGTTGTTCCCCTTTTTGTCCA
TGAGTTCTCATCATTTAGCTCCCACTTACAAGTGAGAACATGCAGTATTTGTTTTTCTGTTCCTGCATTAGTTTGCTAAGGACAGTGGCCTCTA
GCTCCATCCATGTTCCTGCAAAACATATCATCTTATTCTTTTTTATGGCTGCATAGTATTCCATGGTATATATGTACCACATTTTCTTTATTTA
TTTATGTTATTGATAGGGAATTTAGGTTGATTCCATGTCTTTGCTATTGTGGATACTGCCCCAGTGAACATTAACATGCATGTGTCTTTATGGT
AGAATGATTTATATTCCTTTCGTTGTGTACCCAACAGTGGGATTGCTGGGTCAAATGGTACTTCTATTTTTAGCTCTTTGAAAAATCACCACAC
TGCTACAAAATCAATGTGCAAAAATCACAAGCATTCCTATACACCAATAACAGACAAACAGAGAGCCAAATCATGAGTGAACTCCCATTCACAA
TTGCTTCAAACAGAATAAAATACCTAGGAATCCAACTTACAGGGGATGTGAAGGACCTCTTCAAGGAGAACTACAAACCCCTGCTTAATGAAAT
AAAAGAGCACACAAACTAATGGAAGAACATTCCATGCTCATGGATAGGAAGAATCAATATCGTGAAAATGGCCATACTGCCCAAGGTAATTTAT
AGATTCAATGCCATCCCCATCAAGCTACCAATGACTTTCTTCACAGAATTGGAAAAAACTACTTTCAAGTTCATATGGAACCAAAAAAGAGCCT
GCATTGCCAAGACAATCCTAAGCCAAAAGAACAAAGCTGCAGGCGTCACACTACCTGACTTCAAACTATACTACAAGGCTACAGTAACCAAAAC
AGCATGGTACTGGTACCAAAACAGAGATATAGACCAATGGAACAGAACAGAGCCCTCAGAAATAATACCGTACATCTACAACTATCTGATCTTT
GACAAACCTGACAAAAACAAGAAATGGGGAAAGGATTCCTATTTAATAAATGGTGCTCCGAAAACTGGCTAGCCATATGTAGAAAGCTGAAACT
GGATCCCTTCCTTACACCTTATACAAAAATTAATTCAAGATGGATTAAACACTTAAATGTTATATCTAAAACCATAAAAACCCTAGAAGAAGAC
CTAGCCAATACCATTCAGCACATGGGCATGGGCAAAGACTTCATGACTAAAACACCAAAAGCAATGGCAACCAAAGCCAAAATTGACAAATGGG
ATCTAATTAAACTAAAGAGCTTCTCCGCAGCAAAAGAAACTACCATCAGAGTGAACAGGCAACCTACAGAATGGGAGAAAATTTTTACAATGTA
TCCATCTCACAAAGGGCTAATATCCACAATCTATAAAGAACTTAAATAAATTTACAAGAAAAAATCAAACAACCCCATCAAAAAGTGGGCAAAG
GATATGAACAGACACTTCTCAAAAGAAGACATTTATGCAGCCAAAAGACACATGAAAAAATGCTCTCATTATCACTGGCCATCAGAGAAATGCC
AATCAAAACCACAATGACATACCATCTCACACCAGTTAGAATCGCAATCATTAAAAAGTCGAAACAACAGGTGCTGGACAGGTTCTGGAGAAAT
AGGAACACTTTTACACTGTTGGTGGGACTGTAAACTAGTTCAGCCATTGTGGAAGACAGTGTGGCGATTCCTCAAGGATCTAGAACTAGAAATG
CCATTTGACCCAGCCATCCCATTACTGGGTATATACCCAAAGGTTTATAAATCATGCTGCTATAAAGACACATGCACACTTATGTTTATTGCAG
CATTATTCACAATAGCAAAGACTTGGAACCAACCCAAATGTCCATCAGTGATAAACTGGATTAAGAAAATGTGGCATATATACATCATGGAATA
CTATGCAGCCATAAGAAAGGATGAGCTCATGTCCTTTGTAGGGACGTGGGTGAAGCTGGAAACCATCATTCTGAGCAAACTATCCCAAGGACAG
AAAACCAAACACTGCATGTTCTCACTCATAGGTGGAAATTGAACAATGAGAACACTTGGACACAGGGTGGGGAATATCACACCCCTGGGCCTGT
GGTGGGGTGGGGGGAGGGATAGCATTAGGAGATATACCTAATGTAAATGCTGAGTTACTGGGTGCAGCACACCAACATGGCACGTGTATACATA
TGTAACAAACCTGCACATTGTGCACATGTACCCTAGAACTTAAAGTATAATAAAAAATAAGCAAGTTTACAAGGGCAAAAATAAAACAAAAAAG
AAAAAGCACCACACTGCTTCCACAGTGGCTGAACTAATTTGCACTCCCACCAGCAGTATATAACTGTACCCTCTTCTCCACAGCCGTGCCAGCA
TCTGTTATCTTTTGACTTTTTAATAAAAGCCATTCTGACAGGTGTGAGATCATATCTCATTGTGTTTTAATTTGCGTTTCTCTACTGAGCTTTT
TCCATATGTTTGTTCGTGGCATGTGTGTCTTCTCTTGAAAAGTATCTAAAACAGTCACTTATCTTTTAAAGAAACTTTTTAAATCAGAAAAAAG
GTGTTTATATTTAACCACGTATTTATCATTTGCAGTGCTGTTCATTCTGTTTCTTAGGTCAAAATTTCTATCTGGTATCATTTTCTTCTGCCTC
AGGCACTTCCTTTTATTATACTGCTGATCTGATGCTGATTAATTCTTTCAGTAGGTGTATGTTTTCATAGCTTTTTATTTTATCTTTGTTTTTC
AAAGATATTTTGAAGAGTATAGAATTTTAGGTAGACAGGCCGGGCGCAGTGGCTCACGCCTGAAATCCCAGCACTTTGGAAGGTCGAGGCGGGC
AGGTCACCTGAGGTCAGGAGTTCAAGACCAACCTGACCAACATGGAGAAACCCCGTCTCTACTAAAAATACAAAATTAGCCAGGCATGGTGGTG
CATGCCTGTAATCCCAGCTACTCCAGAGGCCGACGCAGGAGAATCACTTGAACCTGGGAGGCACAGGTTGCGGTGAGCCGACATCGCACAATTG
CACTCCGCCCTGCCCAACAACAAACAAACTCCGTCTCAAAAAAAAAGAATTATAGGTTGACAGTATTATTCTTTCACATCCTTAAACTATGTTG
TTCCACTGTCTTCTGATTTGCCTTGTTTCCAAGAAGTCACCTGTCAATCTAATCTTTGTTCCTCTGTATATAATTTTTTTTTCTCTCTAGCAGC
TTTTCAGATTTTCTCTTCCTCACTCGTTTTAAGCAATTTGATTATATGGATATTAGCATAGTTTCCTTCATGTTGCTTGTGCTTGGCGTTCATC
GAGATCCTTAGATCTCTGGGTTTATATATTTAGTACGTTTTAAAACTTTTTGGCCATTATTTTTTCAAATATATTTTCTGTCCACTCCTCTTCA
TCTTCTTCTGGAACCCCAGTTGCACATATATTTGGCTATGTGAAATTTCCTACAGCTCACTGATGACCTGTTTTTTAAAAAATCTTTTTTTTCT
CTTTCATTTTGGAAAGTTTTATTACTGTGTCTGCACGTTCACCAATATTTTTGTCTGTAGTGTCTAATATGTTCTTAATTCCATCTAGTGTATT
TTTTCCTCTTAGACATTGTAATTTGCAATTTCTATAGATTTGTTTGGGGTCTTTTTTTTTCATATCTGCCATGTTACTCCTTAACATACCAATG
CTTTCTTCTTTTTCTGATCATATGGAATATATAATAGCTATTCAATGTACTTGTGTACAAATTCTGTCTTCTGGATCTATTTTTGTTTAGTTTT
TGTCCTAATTATGAATTATATTTTTCTATTTCTTTCCATGCCTGTTAGTTTTTTATTGGATGACAGGTATTTTGAATTTTGTATCGTTAAGTCT
TGGATTCCTTTTTTTTTTTTTTTTTTTTGATGTGCTTTTAAATACGTTTGAGGATTGGGATGAAGTTAAATTTGGGAACAGTTTGATTTTTTTG
ATTCTTTCTAAACTTACTTTTAAGCTTTATCAGAGAGACCAGACAAACCTTTACTCTAGGGTTAATTTGACATCATTGCTAAGGCAGTACCTTT
CTGAATTTTCAACCTGATGCTCCATGTATTACAAGATTTCTTGAACTATTCCAGCCCCATATGTGTTACAATAATTTTTCTGCCTCCACCTCTG
TGGTGGTTCTTTTCCCAGCTTTGACATATTTCCTCACACACAGCACTCAGCTGAAGACTCCAGTGATCTCTGAGTGTATCTCTGTTTGCGGTTT
CTTCCTTTCCGGTACTCTGCTTGTGACTTTTAGCCTCCTTGGCTTCCTCCAATATTTAACTGTCTCTCCTCAACCTCAGAGATTGCCAGGCTCT
GTTGGGGTTCCTCCTTCCTGTGCTGCAGCCTGGGACTCTTTAGGCATTAAGCCAGAGTAATTACAGGGTTCACCTTATTTATTTCCCTTTTCTT
AAAGATTACTGTTCTGTGCTGCCTGTTTTCTAGTGTCTAAAAACCATTTCTTCATGTATTTTAGATATTTAAGCTTCAAACCAGTCTGTTTTAC
TCTATCGTTACCTGAAGCAGAAGACTGACTTCTGTACTCTTTCATTTGTTAACCAGAGTAAATCCTTCATTATTCACATAATAAATTAATAAGG
ATGATGTTTTTCTCACAGGGACTAGATTAGGCAATATACATGAAAAGCATATTATTGACAGTAAAGTGTAAGATGCTACACAGATGTTTATCAT
TGCTATTACAAAGGAGATAACCCCGTTTTCCTGCAGTTAGGGAAGTTCTATATGGGAGTAAGGCTGAAAGGGCCAAAAGATATAGGTATTGTTT
CTGAAAAACTGCCTATGCTTCTATGCATATAAGTATGTCATGTTGCATATTTTTCTGTGCTGTATTAATTCATGCATTCCTTTATCAACAGATA
CTTATTAAACACTCATATGTCAGGCATTGTTCTAGGGACTAGAGATCTCTCCCTTCAAGGAGCTTATTTTCTAGTGGTATATTTTCTGTTCTGT
GTCTTAGCTATCCACTTTTTTCATCTGCCTGGACACGTGACTTATTCTGTCTCTGGGCCTCTGGTATGAGTGCTCATTTCATTCTGCCTTATAA
CTCCTATTTTCTTCCCTACTTTATCTGACCTTCCTACCTTAGCTTGTTCATTCTTTCCTTCAATCCAGTTGTCATGAAATCTCTTTCTTTCCTC
TACTAATTTTTTTTTTCTTTCTTTCTTTCTGAGTAAAAGCCAGAGATCTGCCCCCCTCCTTACCTCTCTGAATTCTTTACTTACTTCTGACAAC
TTGCTCATTTCACTCCAGCTACATTGACCTCCTTGCCTTTGTTGTGTTTTGAAAACACCGGCGTGGTCCTACCGCAGGAATTTTGTACTTACTG
TTCCTTTTGCCACATTCATCATTCATATGTTCATGTCTTCCCCATCGCTTCCTTCAAGTTTTTGCTCAGTTGTCATCTTTTTACTGAGTGTCCT
TCCTGACCTCTCCCTACTTTAAAATGTTATCCTTTTTTCTACTACCTCTCCTGTTCCTCATTCCTACCTTATTTTTCTGGATAACACTTATTGC
CTTCTAAATTGTATCGTATAATTTACTTCTTTGTTTCTTCTCCGTTGTCACACTAGCATATAAATTCAGTGAAGGTAGAGATTCTTTTACTGCT
GACAAAAGCATCTAGGACATTTCCTGGCACTGATAACGATCTGCATAAATATTTGTTGAGTGAATGAATCTCCTTGGTAAAGTCCTTTTTTGTT
TGCCTGTTATATTTATTGAATAGACTTCTTTGTTTCATGTACTTTAATTTCAAAAGTGAATGGCTGGAACATTTTATATATTTTCTTATAAATC
ATTTTCATTGCTTTTTAAGCTTATCACATATTTTGTTTTATAAATATGTAGCCTTCGTGAGATAAAAGATTCTCCATCCCTGTTTACGTGTATA
CTTAGATGACAACTCTAATGGTCATAAATAATTCCAACCTTATAGATAACTCAGGAGAAGATGAGATTATAAGTAGACTTTAAATCCCATTCAG
AAACCCAAGGACAATTCAAAAGGAAAATAATCATTCCAAATATAATATTCTTCTTATTCTTAAGAAGTTGTTAGTATTTTGAATTTTGAATTTT
TAATACAGCTCCCATAGAAAATACTTTAAAATGAACCCGGTAAGACTTCCTCATTAAGACATATAGACACATATGCTCCCACTCCCGTAATCAA
ATTTGGAAGTCAAATAAGCTCTGAAAACCAAAACATTCTTCCAAGTTTATTGCAGACTCATTTGGTAGTAAAAACCAATCTGACCTGATGTGTA
GCTGTTTATAGTCTTTATTTTTCCATTTGGTGAGTCCAATTATATATTTCGCTGAGGAAATACTAACATGTTTGACTACATTGTGTGCCCCAGA
CCTGCTTAGAGTATTATCTAATATGCAGTATAGGCCATATATTGCCTTTTTAACATCAAAAATACCCTAAATTCTGAAATGCATGTAGCCCCAA
GAGATTTGTATAAAGCATTGAGGGCCTGTGTATTTTTTTTTAATTTACAAGTGAAGCCAGATTCCCTAGACATTAAGTGCCTAACTTTTGGTTG
TTGTTGCTGCTGCTGTTACTGTTTTTTCTCCAGCTTCGCAAACCTGGGTATACTTCATGTGACAAAGAAAAAAGTATTTGAAACACTGGAAGCA
CGAATGACAGAGGCGTGTATAAGGGGCTATAATCCTCGACTCTTGGTGCACCCTGACCTTGCCTATTTGCAAGCACAAGGTGGAGGGGACCGGC
AGCTGGGAGGTAAGCATCATTTTCCTCGCCTTCATCCTCCAAGGGGTCCAGGCTTTGGTTTTCATCTGTATGAATTATATGTTCATCTGCATCC
CTTCCAGTCTCTACCCCACACTGCTGTCACCTTTTCCTTGCTCACAAACCTTTCATCGCATCCTGCTACTTCTAGGATAAAAACTATAGCTCCA
TAGCCTGTCATACAAAGCCCTGCTTCTCTGGCCCCAGCAACTTTCAGCCTCATCTCCAGCCCACCTTGCATCCTCCTCTCGCCATCTCTGCTCT
CTGAATAACTGAGGCATATTTATACCTTTGTATGTGTCTTCCTTTACCAAAAACACCCCACCGTTTCCATTTCACCCTCTCGAAAACCAATGCA
GAAATCAAGACTTTGTCTAAAGAACACCTCTGTTTTACCTTCCCCAACTCACCTTATTATAAGGCATTTTTCTTTCTTTGGGCTCTGCAGACAC
ATTATAAACTATTATCTTTATCATAAAAATTGTATGATTTTCCATTTTCTTGCCTTTTCTCAAATTTTTTTTTCAACCCCATTCCCTGTCCCAC
CACATGCCCCATTTCTACTTTTGTATTTTCTCCTTTACTCTCCCCGTCCACTCGTCATCTGATACTCATGTGATTCAAAACCGATTATCAGTTT
TCCACTAAATATACTCCATCTTCTGATACCCAGACTCAAAACCTTCCCATCCTCACTGACTCCCCCTCCTCGCCTGTGCTGCGAGGCTGTGGTC
CAGCCATTCCGTGCTCTCTGGCATTCCACACTACATTCCATTTTCATTGTTCCACTTATAGTAGTCTTCTCCCTGGTCTCTCTCCCTCCCCACT
TCTAATCAAGTTTACATCTTGTAGCCAGATAGATATTACTGAAGTTCTGTATCTTGTTGCTGCTCCGTGTCTCCGTTTTGCCTACAAAATCCAA
ATTCTGTAAGTTGGCTTTTTGGATCTCCCAAGAGCTGGCCTCAGGCTGCATTTCCAATCTATTTCCCGTTTTGCCCCTTCAGGTCAAAGTCTAC
TACTTGGTACGACCACCATCATTTCTCACTTTCCTCCCTTGCCACATTCTTTCTATTTTTGCTCTCTCCTCTTCCTGGAATGCCTTTATCCACT
CTCTTATATGAATGTGTTCAAATTCTGCCACCTTTTCAATATTCAGAGCAAATACCTCTTCTGCTCCAAAGCCCTCTGAACTCTCTCCTTCTTT
GAAATCCCACATCACTTCATTTGTAGCTCTCTTGTAGTACTTCTCTCTTGAATCACTACCTTTTATGATATTATAGCTTAATTATGACTATATC
CATCAGCGTGAAAACTCCAGCAGGAGGGAAGGAAACAGATGTCCCTGAGTCATTCAGTTCCTTCAGAAGTTGTTTTTGAGTACCTACTATGTGC
CAGGCTGTCTGCTTGGCACTGTGGAATGAGCTCACTCCCTGTGCCCAAGCAGCTCTTGGAATAATTTGCACACTTCTCAATACAGCTGGTGCCT
AGTAGGGTTATTCAATTCCATTCCATCACATCCTGTGTGCCTTTAACTCTTACTCTAGAGAGGAAAGGGAAAGGTGGGGGAAGGACAGGAGAGG
AGGGAGGCGATCTGATACACGGAAGAGAGTTTTTACTGTAATCCGGAGAAAATCCACGTCTTCTTTGATGCCTTTCATATTGGATGACATAAAT
GAGACTGTTTTTAACACTTTATTACCAATATGAAGAGTTTCAAAAGAGCAAAAATGGGTTTTTATTGTAAGTTTACATTATTTGGCCTTTATAA
AAGCATGGTCTTTTAAATGTTCACACTTCCCTGGGCATGAATGGACTGTGCTGTATGGCCCTAGATCGGGAAAAAGAGCTAATCCGCCAAGCAG
CTCTGCAGCAGACCAAGGACATGGACCTCAGCGTGGTGCGGCTCATGTTTACAGCTTTTCTTCCCGATAGCACTGGCAGCTTCACAAGGCGCCT
GGAACCCGTGGTATCAGACGCCATCTATGACAGTAGTGAGTACTTCACTTCCAACAGGGGGCACACCAAGAATACACTTCCAGCCCTGCCCTGC
CATTTACTTGCTAGCTGAGTCCTCCGGAAGGTAACTTAATCACTTTAAGCCTCAGCTTTATCATCTGTAAAATGTGAATAGGAAAATCTAGCTT
GAAAGGTTGATGTCCAGGTTAAATGAGGTATTTTAAGAGGGGCTCAATACATGTAATTCTTTTTCCTTTGATGTACATTTTTTAGTCTTTTACA
TGAAATGTCACATCTCATCTTATTTATGAATGTCTTGCTATAAAGATATATCGTTGATACTTTTAGAACGGAGAATAATCCCAATTTTCTTTGG
TGGTGGGGGGACTCTGGAAAGGAGTTTATAAAAGACAACTATTATTCTTACCCATTTTCTTTCCCATATGTTACTAGCTTTATCAAGAGAGAAA
CTTGAAGTTAAATGAATGAGTAGCATAGACAGCATGTGACTTCAAAATCCTATTTCAAGCCCGGTTTGGCGTCATGCCCTGTAGTCTCACCTAC
TTGGGAGGCTGAGGTGGGAGGATATGGAGTTTCACCCCAGCATGAGCAACATAATGAGACCCTATCTCAAAAAAAAAAAATATATATATATATA
TGTATATATATATTAATTTATAGTTCCTAGGACTTTTTAAGAGTTTTACAGTAACCTTCAGCATTTACCATGCAACTTTCTTGTTTGATTATGC
ATAGGATTGATTAGACAGGTATTTTCAAAAAGCCTGCAAGCATAGTACTCAGACTCATTGAAAGTTTATAGAATTTGGCCTGTGTGGAAAACTC
TGTGCTCCAAGTACAACAACTAACTGAATTCTCTAATTTAAACAAGTTTGAAGGGAAGTGAAAGGTTATAAAATGATACAGACTCATACCGCAG
AATCACCTTAAAATGCAGCTGTTGGAGAAAAAAAAAAAGGAAGCTTTATGCTATTACCACAAGAAAGTTTTGCCTCTTCACCACACAGAATCCT
AATTTAGTTTGGGAAAAAGAAACATAACATCCCCATTTTCCTTACCTGTAAAATAAAACGTAGTAAGTAGTTTAAGAAAACTGTACTTTTCCTA
AAGTTTTTACGACTCTTGTAAATAGAATAATAGCAGAATCATGGAAACAAATTTGGAATTGAAGTCTTCACCTCATTGAAAGCCCAGAAATCAA
TTAGCTTGGTACTTCCCAATACATCAGAAAAGCTCTTTCTCTTTTTGTGTCAGTCTCTCTGTGTTAGAGGGACAAACAAATACCTGTTCCTTTC
TACCTCATTTGTTTTGGTGATCATAAATGACCCATGCCCAAGAAGTGTTCCGAGAATTTCAGAAGCCATTCTTGTTTTATTTACTTTGTTAGTG
AAAGCATGTATTTAAGCTTTGTTTTGGTCATGTGTGCTAACGGGAGGGTCCTACCTAAAGGACTGGCTTGTTCAGCTGAGGATTAATCATCTTA
TTTGTTGTTTTTCCCCTGTGAACAGAAGCCCCCAATGCATCCAACTTGAAAATTGTAAGAATGGACAGGACAGCTGGATGTGTGACTGGACGGC
AGGAAATTTATCTTCTTTGTGACAAAGTTCAGAAAGGTAAATACATTCTGTGATCTCTGATCTCAAGAGGTGTGATCTTGACACACTACAGTTC
TGAGTGTGTCTGTGAGTCACATTTCAGCAGTGGACAAGAAACATCCCTCTGCTGCCACAGAAAGTCTTAAAAAGCATTTACCTTTTACCTCTTC
CAAATGTAAATTGTCTGTGTTATTTTTTCCTAAGTCAACTAAATCACTTTTAGATTTCCCATGGAAGTAAAAAGAAATAAGAAAACCGTGATAA
TTATAATCAGTGACTTTCAGTATCTTTATACATTAAAATTAATATCTGTACTTTGTTAAACAAAAATAAATAATAGCCCATTCTTGACACACAT
ACAAACACACACACAGAATAATGTTTTACATAGTATTAAATTGTTTTTATTTCTTTTGCAATGCTTTGATATAACACTGAGAGAACTTCTCTGA
ACACTTCCACAGATGTGATGTTTAAGAAAAGGAAAGAAAACAGGGAATACAGGGAAATTCTCAGAGCACATTTCTCTCACAATTTTCCCTTTGA
GGAAAGGTTCTTGGCGTTTATCTTTCATTAGATATATTACAACATTGATAGATTAGTAAAAGCTGAGGAAAATGTCTTATTTTCTCAAACTGCA
TTCTTTTTTTAAATAAAATGATAGTTGGAAAGTGTAGTATAATATCTATTTACTACAGGGTGAGCATTTTCAAAGTAAGCAGATACCTAACTGA
AAATATAAATAAGTAAAATAAATCAATAAAAGACATGTGGCTTTTAAAAAAATTATCATATGAGATTAAATTCTCTGAGGTATATAGTCAGAAA
AAAATGGGATCAGATTTTAAAGTAAACACATATATACTTTATCCAAAAAAATTTACAGGCCAGGCATGGTAACTCATGCCTGTAATCCCAGCAC
TTTGGGAGGCTGAGGTGGCAGGATTGCTTGAACCCAGGAGTTTGAGGCTGTAGTGAGCTATTATCATGCTGCTGTACTCCAGCCTAGATGACAG
AGCGAGACCCTGTCTCAAAAAAAAGAAGTTTACACTTTCTCTGCCTACACTAGTGTCAGCCTTCTTCCAATCCACTCTGTCCAAGTAATACTCA
TTTCCAGATTCCTGGGTGTACATACTGTGAGTAAACACAATCTGAGTACCTTTATTTGTAAATGAGATCCTTAGTCATTATCATTACATTGGAA
TGGCTTAGTAAGAAATGTCTTGTACTTGGCTTAAAGAATCTCAATAACTAGGTCCAGTGTAGTCCTACCTGAAGGCAAAGGAGATTGTAAGATG
TTTTATCCATTCCTACTATTTCTAAAATTCTACTTACATAGAGATAAAAACTTTGAACAAAGTCAATAGTCTGGCCCTTTCTATGAAACGGAAA
GTGAAGCCACGAAGATGCCAACCTAAGTTACATTGTCACATTTCTGTAGAAGTGAATAGAGTATTTATAAACATGCTGAACATGGAGTATTTAT
AAACACACTCCATGACTAGAGTGTGCAATTTAAGCATTGCATTTTAATTAGAAGGGACCTGATTATTATAGTATTTTGACCAATTACTCAATAT
GAACTAAGAGACTATTATAGTGAGACAGTTTAATAGTGTGTTAACAGCCTGGACTCTGAAATTATACTGTCTATGTTAAAATCCTGGTTCCTCC
AAGTACTAGCTGTTCACCCTTGGATAAGTACTTAACCTCTTTGGACCCCAGTTTCTCTTTTGCAAAGTGAGGATAATAATAGTACCCATCTACA
TCACAAGGTTGTGGTGAGGATGAAATCAGTTAATATGTGTATATATGAAGCACTTAGAATAGCATCTGCCATACATTAATAGTAAAACTTATAT
AAGTTAATTATTTTTATGTACATCTATATATTGAATTGCACTGCCTCGTTGGATTCTAATATTCTTCCACCATCGTTCGTCTTTCTCAGGATTG
GCTTAAGGATGGAGAACCAGTAAGGTCAAAGATTAAAACAAAATTCAAGTTAACTAGGTAAATCTAGTATTCTGTCATTGTGAATGAAAGTTGG
GGCGCATTACTGTGGTAAATAATGAAAAAAGTCAGTGATTTGTGAAGATTTTAAAAGACTGAACCTTTTGATCTTGTTTTTTAAAAGTGTAAAT
AGATAGTAGGTAGAATATTAAACCAGTTTTATTTTTCAGCATGTTTATAAATACTATTTACACTATGTGAAATTACACACTTCAATGTGATTGT
TTGCAGATGACATCCAGATTCGATTTTATGAAGAGGAAGAAAATGGTGGAGTCTGGGAAGGATTTGGAGATTTTTCCCCCACAGATGTTCATAG
ACAAGTAAGTGATTTATTATTATTATTAATCCTTATTATTTTTAGAGATGGGATCTCACTCTGACACCCAGGCTGCAGTGCAGTGGTACAATCA
CAGCTCACTGTATCCCCCAACTGTTGGGCTTGAGGGATCCTCCTGTCTCAACCTACCAAATATCTGGGACTACAGGCATGCTACCATGCCCAGC
TAGTTTCTTCAGTTTTATTTTTTTTGTAGAGATGATGTCTTGCCATCTTGCTCACGCTCGTCTTGAACTCCTGGACTTAAGCGATTATCCCACA
TTGGCCTCCCAAAATGCTGGGATTATAGGCATGATCCATATTACTATCATCATTTTATACTTTCTGCCTTATTGAAATTTAGTACTTAGCTTCC
ATCTATAAAAAAGAAAAATCATAAATATTTAACTTCCATAAAACAGTACTATTTTTAAGACTCATGCAGGTCATCCAAAAAAGTTACCTACTGT
AGAGTTATGGGCAAGGTTCTGTTACATCGTTTGCTCTGAGATGTTGGCAAGATATTTAATTGCTCCCTAATCCCCTAACCCATAAGACAGTGCC
CCACAGGTCCTTTAATGAAAATGTTTTTAAGTGTTCTACCAAAGTAATTATGATGAGACCTATTTTATGATAGCACCTAATTATGATAACACCT
TAAAAGGGTGTTCTAGGAGTCATCTTCTAATTGAAGCTGATGTCATCCTGTTAAATGAAGATTTCCCAATGGCAACTGATTAAATGATCAGTTT
AATCATTTAATAACATGATAATGCTGAGCCCCCACCTTAATTATTATTCTCCTATTTACAAGTAATTGAAGAGTATTCTCCTATTTACAAGTAA
TTATCTGGATTGGAAACAATCCAGATATTCATCAGTAAGTGGCCAGTTAAATTATGGTCTGTCCTTTTGATGAAATATCATGTAGTTATTCAAA
AGAATGACTACTCTGCGAACTAATATAGAAAGATCTCAGTTATACAGTCTTAAAATATTTTAAAAGATGGGTGTAGAACAAAGTGTCTGTAGAA
ATGATTCTATGTTCATGCATAAAGAAATTCTGGAAGGATACCTAACAAATTAATAACAGTATTTACCACTGGCTGGTACACTGTAGCAACTGCA
TGGATGGGGAACAAGAATGTCAAAGAGAATACCACTGTATGCTTTTTTATATTTTATAAGATCTTTATCCCTGTGAATATATTACATAGTCAAA
AAATCAAACTGAAAAATGCTTAAATCCCTAACATACCACCAAGAACTAATGTGTTATCATGCCAATGTAAGCAATGTTTAGTTTCTTCTCTTCA
TCACTGGCCTGTACCCATCTCTCTCTTTCCATACCCAGTCTTTCTGATGTTGCTCTAAATAATCTTGCAGGCTTTTAAGGCTGCTTTGGAAGAG
AAGAAAGTATATGCAGTGAATTTTATAAGCATGATATTTACAACTAGAAGACATTTGAAATAGACAAATGAAATAATAAAGCTTCGTAGTACAT
TAGCATAGCATTGTATTTTGATTTTACAGTAGCAATTTCATAAAAATGTATCGGTATAGAATTCTGAGTTTGGAACTTTCTCCAGGACACGGTG
TTTTTTAGTACACTGTGTCTAAACATTGGGTATAAAGAAAGCATAAGGAATGTGTTTAATGACTAGCATTACTGCAAAAAAAAAAAAAAATGTA
GTCCTACACCAACATGTGGTTCTTCGTATCCTGCAGTTTCCCATTGTCTTCAAAACTCCAAAGTATAAAGATATTAATATTACAAAACCAGCCT
CTGTGTTTGTCCACCTTCGGAGGAAATCTGACTTGGAAACTAGTGAACCAAAACCTTTCCTCTACTATCCTGAAATCAAAGGTAAGTCAGTTGT
TTAAAATCTTATGCTCATATTTTATTTTATTTTATTTTTGGTTTTTGTTTTGTTTTGTTTTGTTTTGAGACAGAGTCTGACTCTGTTGCCCAGG
CTGGAGTCCAATGGTGCAATCATAGCTCACTGCAACTTCCAACTCCTGGCCTCAAACACTTCTCCTGCCCCAGCATCTCAAATACCTGGGACTA
CAGGTGTGCGCCACCATGCCGGGCTAATTTTTTTTTTTTTTTACTTTTAATAGAAATGAGGACTTCCTACATTGTCCAGGCTGGCCTTAAACTC
CTGGCCTTAAGCAGTCCTCCGGCCTTGGCCTCCCAAAGTGCTGGCATTACAGGCTTGAGCTGCCATGTCTTGCCATTGTGCTTGTTCTGAGAAG
AGGACAAGCTAATTAGAAAAGATCAGTTAGTCACCTCCTTTGAACAGCTTTCTAGTAACAGGTCCCTGGATCCATGGTGCTTATTTTTAGAAGA
GACAGTAGTATATTATTTTGAGGTCATGGAATTAGTCTAGCTTTTTAAAATAATGTTATTTCCACCAAGCTTATATGAGATTTACACATTGAGA
ATTTGTCTTTGAATTTGAGAATCTAACATTTATTGACTAACTCAGAGTTCAAGCAGCTGAGATCAAAAAATCAGCCTTGCTGTTTAGGAGAATC
AGCCACTTAGTCAACCAGCCTCAGCCGGTGGAACAGAAAACTTGCCCAACATGCACTGTGAAACTATCAAAATGAGTAAGGCATCATTCTTTCT
CTCACAAAAGATGCTTTAAGAAAGGGCACTCATAGGTTTCTTAAATAGTTATAATGTAAATTATGATTTTAATAAGTACCGAAAGTGAAGCATC
CATAAAAATATAAGAGAATGTTGAAGAGAGAGCCCTTGGTCCCTTTATTCTAAAGCAGCCTCCAAAAAGAAGGCTTCGTCAAGGAGATATCATT
TGGCCCTCAGCATTTGGGGTATATTTCAGCAGTTGGGTGTTAAACAGAGAAGGAGAGGGTTCTCTAGGTTTAAGTTACTTGAGGAGCTTAAAGA
GAGAGGTAAACACACCCTTTATGCAGAGGGAAAAAAGTGTTCTGAGCGCTCGGATGGACATGAAGGCCAATGTGGGTAAGGAAGGCCAGGGCCA
GGCTAAACAGGGCTGTGAATGGCAGCTTAAGAATTTTGCATTAATTCTGGAGAGGGATAGCCGGGCCTGCCTAGGTTTGGAATGATGGCTCTGA
CTTCTTTATGTCACCACTGAGGGTACCATTTAGGAGCCAGATGATGAGTCAGAAGAGTAATGCAAGAATCCAAGTGAGAAACAGTGCAGTTAGT
AAAGGTCCTAGTGCCAGGAATGAAAGGAGATGGTGGACTTAAGAGAGAGTGCAGTGATACTGTGATACAGTGATACTGTCAAGAGCGCCTAGAG
GTCGGAGGTGTACGAGAGACGGGGAGTCATAGATGGGTCTAAGACTCCCTGACCAGGAAGCTTCTGATGTGGTTGACTTGATGAAAGGAACAAG
GGAGCAAAAGTAGATTTGGGTGGGAAAAGAAGGAGCTCAGAGTAAAACATGTTATATTTTAGGTCCCTGTGGGAAAATCAAGTGTAAGTTCTTA
GTAGGCAGATCAAAAGGCAGAACTGGAACTCAAGAGAGGACAGGGTGAGAAATTTAGCATTAGAATCATCTCAAAACGAGATATAACAGGCACC
AACTGAGTCATCATTCAAAGGTATTTCTTAACCTAGAGTCCACTGACCCCCAAAGAGTCTGTGGATAGAAATGTACTTTGATATCATCAGCTTC
TTTTATAATCCTGTTTTCTATTTTACATACTGAAAACCATCATTCTGAGAAGACATCCATAGAGTTCACCAGACTGCAGAAGGGGCTGTGGCAC
AAACAAAATTAGAAACCCCTCAGTTGGAGGGAAGTGAGACTGGGAAGGGAGGTCAGTAAAATGATTGCCACGGGGCACCCAGATCCTTGCTGAG
GTTGGAAACCGCAAAGGTGCAGCAGCAACAAGCCACATGGGTGGGTGTTGTCTCCTGTCAGAGCAAGATCTGATAGGTAATCTTTGAAGAGGGC
TTTCCTTTCCTAGAATTCGTGCTCCCACAGGAGTCTTTAGAAAACGCTTCTACAAGTTCTTCTTTCTTTTCTGAGGTTGTGCCCTCTCCATTGT
TTGGTCAGATTTGTAGTGCACTCTATATCCTGGTGGTTTGAAAACATTGTCAGCACAATAACTTTTCTTTCTTCACATGAGATTTCATACAAAA
TCCCAGTGTATCGACATGGTAGGCTTTGATTAAAATAGGGGTGAAGGGCCCTGCCAGCTCGGCCTTCCCAGAGGCTGTCCCACCTCTCCTTACT
TCCCTATCAGGAGCTGCTGGACCTCTTTTGAATGCTTCTGCCTACACAAAATGATGTGAAAATCACTGCTTTAATTTAAACACCCTTCTTTAAA
AGTAGACACAGAAGAAAAATATGCATAATTTTTTTAGACTTTCTGAGGAAAAAAATAGATTTCTTCTCCAGAAATATGTCTTCAAATAATGCCA
GTTTTTGCTAACACAGAGAATCATGAAGAAAGAAAACCCAGGTTACCTGTATGTGTATGAACCTACAAACATTCAAAGCTCAGCTTGGTATCTA
GGCTGCCTGTCTCCCTTTCCCAGTGGTATCTATGCCTCAGAAAAGTATCTTATAGTGGCGGTATATTCAGGTGATTACTTTAATGCCTCGTTAT
CATAGTAGGAACTATCCAATGCAGGGATTAGAGAATGGGGTCACCTGAAACTTCAAAATTGCACCTGTATGCTTCATGCACCTACCTTCCAGGC
TGTCATTGATGAAAACCTTCATACAATACAAAGTTTAACAACTGTCTGGAGAAATACTTACCCATTGAGCTCTGAAGGAAAGAATGCACAACAT
ATATATATATGAGGACAACAAATTACTTGATTCTATAGAAACCTTCTGGATTATAAAAACTCAAGATAATAGGACCAAGGAGAAATAATCTGAC
CAGGCAATATAACTGGTCAGAAAAGTAGTACGCCATTTAGCATTAGAGTAAGTACATAGAAATGGATCCAAACTGTACAACCCAGGGAAACATT
GCATACATTTCTCAGAAGTCATTGAACACACTTTTCCTTACCCCCAGTCTCTGT
HUMAN SEQUENCE—mRNA
GGCCACCGGAGCGGCCCGGCGACGATCGCTGACAGCTTCCCCTGCCCTTCCCGTCGGTCGGGCCGCCAGCCGCCGCAGCCCTCGGCCTGCACGC
AGCCACCCGCCCCGCTCCCGGAGCCCAGCGCCGCCCAGGCCGCAGCCGCCCGGCCAGTAAGGCGGCGCCGCCCCCGGCCACCCCGGGCCCTGCC
GTTCCCTCCCCCGCGCTGCGCCATGGCGCGGCGCTGACTGGCCTGGCCCCGCCCCGCCGCGCTCCCGCTCGCCCCCACCCGCACTCGGGCCCGC
CCGGGCTCCGGCCTGCCGCCGCCTCTTCCTTCTCCAGCCGGCAGCCCCCGCCGCTTAGGAGGGAGAGCCCACCCGCGCCACGAGGCCGAACGCG
GACTCGCCACCCGGCTTCAGAATGGCAGAAGATGATCCATATTTCGGAAGGCCTGAACAAATGTTTCATTTGGATCCTTCTTTGACTCATACAA
TATTTAATCCAGAACTATTTCAACCACAGATGCCACTGCCAACAGATGGCCCATACCTTCAAATATTAGACCAACCTAAACACAGAGGATTTCG
TTTCCGTTATGTATGTGAAGGCCCATCCCATGGTGGACTACCTGGTGCCTCTAGTGAAAAGAACAAGAAGTCTTACCCTCAGGTCAAAATCTGC
AACTATGTGGGACCAGCAAAGGTTATTGTTCAGTTGGTCACAAATGGAAAAAATATCCACCTGCATGCCCACAGCCTGGTGGGAAAACACTGTG
AGGATGGGATCTCCACTGTAACTGCTGGACCCAAGGACATGCTGGTCGGCTTCCCAAACCTGGGTATACTTCATGTGACAAAGAAAAAAGTATT
TGAAACACTGGAAGCACGAATGACAGAGGCGTGTATAAGGGGCTATAATCCTGGACTCTTGGTGCACCCTGACCTTGCCTATTTGCAAGCAGAA
GGTGGAGGGGACCCGCAGCTGGGAGATCCCGAAAAAGAGCTAATCCGCCAAGCAGCTCTGCAGCAGACCAAGGAGATGGACCTCAGCGTGGTGC
GGCTCATGTTTACAGCTTTTCTTCCGGATAGCACTGGCAGCTTCACAAGGCGCCTGCAACCCGTGGTATCAGACGCCATCTATGACAGTAAAGC
CCCCAATGCATCCAACTTGAAAATTGTAAGAATGCACAGGACAGCTGCATCTGTGACTGGAGCCCAGGAAATTTATCTTCTTTGTGACAAAGTT
CAGAAAGATGACATCCAGATTCGATTTTATGAAGAGGAAGAAAATGGTGGAGTCTGGGAAGGATTTGGAGATTTTTCCCCCACAGATGTTCATA
GACAATTTGCCATTGTCTTCAAAACTCCAAAGTATAAAGATATTAATATTACAAAACCAGCCTCTGTGTTTGTCCAGCTTCGGAGGAAATCTGA
CTTGGAAACTAGTGAACCAAAACCTTTCCTCTACTATCCTGAAATCAAAGATAAAGAAGAAGTGCAGAGGAAACGTCACAAGCTCATGCCCAAT
TTTTCGGATAGTTTCGCCGGCGGTAGTGGTGCCGGAGCTGGAGGCGGAGGCATGTTTGGTAGTGGCGGTGCAGGAGGGGGCACTGGAAGTACAG
GTCCAGGGTATACCTTCCCACACTATGGATTTCCTACTTATGGTGCGATTACTTTCCATCCTCGAACTACTAAATCTAATGCTGGGATCAAGCA
TGGAACCATGGACACTGAATCTAAAAAGGACCCTCAAGCTTGTGACAAAAGTGATGACAAAAACACTGTAAACCTCTTTGGGAAAGTTATTGAA
ACCACAGAGCAAGATCAGCAGCCCAGCGAGGCCACCCTTGGGAATGGTGAGCTCACTCTAACGTATGCAACAGGAACAAAAGAAGAGACTGCTG
GAGTTCAGGATAACCTCTTTCTAGAGAAGGCTATGCAGCTTGCAAACAGCCATGCCAATGCCCTTTTCGACTACGCCGTGACAGGAGACGTGAA
GATGCTGCTGGCCGTCCAGCCCCATCTCACTGCTGTGCAGGATGAGAATGGGGACAGTGTCTTACACTTAGCAATCATCCACCTTCATTCTCAA
CTTGTGAGGGATCTACTAGAAGTCACATCTGGTTTGATTTCTGATGACATTATCAACATGAGAAATGATCTGTACCAGACGCCCTTGCACTTGG
CAGTGATCACTAAGCAGGAAGATGTGGTGGAGGATTTGCTGAGGGCTGGGGCCGACCTGAGCCTTCTGGACCGCTTGGGTAACTCTGTTTTGCA
CCTAGCTGCCAAAGAAGGACATGATAAAGTTCTCAGTATCTTACTCAAGCACAAAAAGGCAGCACTACTTCTTGACCACCCCAACGGGGACGGT
CTGAATGCCATTCATCTAGCCATGATGAGCAATAGCCTGCCATGTTTGCTGCTGCTGGTGGCCCCTGGGGCTGACGTCAATGCTCAGGAGCAGA
AGTCCGGGCGCACAGCACTGCACCTGGCTGTGGAGCACGACAACATCTCATTGGCAGGCTGCCTGCTCCTGGAGGGTGATGCCCATGTGGACAG
TACTACCTACGATGGAACCACACCCCTGCATATAGCAGCTGGGAGAGGGTCCACCAGGCTGGCAGCTCTTCTCAAAGCAGCAGGAGCACATCCC
CTGGTGGAGAACTTTGAGCCTCTCTATGACCTGGATCACTCTTGGGAAAATGCAGGAGAGGATGAAGGAGTTGTGCCTCGAACCACGCCTCTAG
ATATGGCCACCAGCTGGCAGGTATTTGACATATTAAATCGGAAACCATATGAGCCAGAGTTTACATCTGATGATTTACTAGCACAAGGAGACAT
GAAACAGCTGGCTGAAGATGTGAAGCTGCAGCTGTATAAGTTACTAGAAATTCCTGATCCAGACAAAAACTGGGCTACTCTGGCGCAGAAATTA
GGTCTGGGCATACTTAATAATCCCTTCCGGCTGAGTCCTGCTCCTTCCAAAACACTTATGGACAACTATGAGGTCTCTGGGGGTACAGTCAGAG
AGCTGGTGGAGGCCCTGAGACAAATGGCCTACACCGAAGCAATTGAACTGATCCAGGCAGCCTCCACCCCAGTGAAGACCACCTCTCAGGCCCA
CTCGCTGCCTCTCTCGCCTGCCTCCACAACGCAGCAAATAGACGAGCTCCGACACAGTGACAGTGTCTCCCACACGGGCGTGGAGACATCCTTC
CGCAAACTCAGCTTTACCCAGTCTCTGACCAGTGGTGCCTCACTCCTAACTCTCAACAAAATGCCCCATGATTATGGGCAGCAAGGACCTCTAG
AAGGCAAAATTTAGCCTGCTGACAATTTCCCACACCGTGTAAACCAAAGCCCTAAAATTCCACTGCGTTCTCCACAAGACAGAAGCTGAAGTGC
ATCCAAAGGTGCTCAGACAGCCGGCCCGCCTGAATCATTCTCGATTTAACTCGAGACCTTTTCAACTTGGCTTCCTTTCTTGGTTCATAAATGA
ATTTTAGTTTGGTTCACTTACAGATAGTATCTAGCAATCACAACACTGGCTGAGCGGATGCATCTCGGGATGAGGTTGCTTACTAAGCTTTGCC
AGCTGCTGCTGGATCACAGCTGCTTTCTGTTGTCATTGCTGTTGTCCCTCTGC
HUMAN SEQUENCE—CODING
ATGGCAGAAGATGATCCATATTTGGGAAGGCCTGAACAAATGTTTCATTTGGATCCTTCTTTGACTCATACAATATTTAATCCAGAAGTATTTC
AACCACAGATGGCACTGCCAACAGATCGCCCATACCTTCAAATATTAGAGCAACCTAAACAGAGAGGATTTCGTTTCCGTTATGTATGTGAAGG
CCCATCCCATGGTGGACTACCTGGTGCCTCTAGTGAAAAGAACAAGAAGTCTTACCCTCAGGTCAAAATCTCCAACTATGTGGGACCAGCAAAG
GTTATTGTTCAGTTGGTCACAAATGGAAAAAATATCCACCTGCATGCCCACAGCCTGGTGGGAAAACACTGTGAGGATGGGATCTGCACTGTAA
CTGCTGGACCCAAGGACATGGTGGTCGGCTTCGCAAACCTGGGTATACTTCATGTGACAAAGAAAAAAGTATTTCAAACACTGGAAGCACGAAT
GACAGAGGCGTGTATAAGGGGCTATAATCCTGGACTCTTGGTGCACCCTGACCTTGCCTATTTGCAAGCAGAAGGTGGAGGGGACCGGCAGCTG
GGAGATCGGGAAAAAGAGCTAATCCGCCAAGCAGCTCTGCAGCAGACCAAGGAGATGGACCTCAGCGTGGTGCGGCTCATGTTTACAGCTTTTC
TTCCGGATAGCACTGGCAGCTTCACAAGGCGCCTGGAACCCGTGGTATCAGACGCCATCTATGACAGTAAAGCCCCCAATGCATCCAACTTGAA
AATTGTAAGAATGGACACGACAGCTGGATGTGTCACTGGAGGGGACGAAATTTATCTTCTTTGTGACAAAGTTCAGAAAGATGACATCCAGATT
CGATTTTATGAAGAGGAAGAAAATGGTGGAGTCTGGGAAGGATTTGGAGATTTTTCCCCCACAGATGTTCATAGACAATTTGCCATTGTCTTCA
AAACTCCAAAGTATAAAGATATTAATATTACAAAACCAGCCTCTGTGTTTGTCCAGCTTCGGAGCAAATCTGACTTGGAAACTAGTGAACCAAA
ACCTTTCCTCTACTATCCTGAAATCAAAGATAAAGAAGAAGTGCAGAGGAAACGTCAGAAGCTCATGCCCAATTTTTCGGATAGTTTCGGCGGT
GGTAGTGGTGCCGGAGCTGGAGGCGGAGGCATGTTTGGTAGTGGCGGTGGACGAGGGGGCACTGGAAGTACAGGTCCAGGGTATAGCTTCCCAC
ACTATCGATTTCCTACTTATGCTGGGATTACTTTCCATCCTGGAACTACTAAATCTAATGCTGGGATGAAGCATGGAACCATGGACACTGAATC
TAAAAAGGACCCTGAAGGTTGTGACAAAAGTGATGACAAAAACACTGTAAACCTCTTTGGGAAAGTTATTGAAACCACAGAGCAAGATCAGGAG
CCCAGCGAGGCCACCGTTGGGAATGGTGAGGTCACTCTAACGTATGCAACAGGAACAAAAGAAGAGAGTGCTGGAGTTCAGGATAACCTCTTTC
TAGAGAAGGCTATGCAGCTTGCAAAGAGCCATGCCAATGCCCTTTTCGACTACGCGGTGACAGGAGACGTGAAGATGCTGCTGGCCGTCCAGCG
CCATCTCACTGCTGTGCAGGATGAGAATGGGGACAGTGTCTTACACTTAGCAATCATCCACCTTCATTCTCAACTTGTGAGGGATCTACTAGAA
GTCACATCTGGTTTGATTTCTGATGACATTATCAACATGAGAAATGATCTGTACCAGACGCCCTTGCACTTGCCAGTGATCACTAAGCAGGAAG
ATGTGGTGGAGGATTTGCTGAGGGCTGGGGCCGACCTGAGCCTTCTGGACCGCTTGGGTAACTCTGTTTTGCACCTAGCTGCCAAAGAAGGACA
TGATAAAGTTCTCAGTATCTTACTCAAGCACAAAAAGGCAGCACTACTTCTTGACCACCCCAACGGGGACGGTCTGAATCCCATTCATCTAGCC
ATGATGAGCAATAGCCTGCCATGTTTGCTGCTGCTGGTGGCCGCTCGGGCTGACGTCAATGCTCAGGAGCAGAAGTCCGGGCCCACAGCACTGC
ACCTGGCTGTGGAGCACGACAACATCTCATTGGCAGGCTGCCTGCTCCTGGAGGGTGATGCCCATGTGGACAGTACTACCTACGATGGAACCAC
ACCCCTGCATATAGCAGCTGGGAGAGGGTCCACCAGGCTGGCAGCTCTTCTCAAAGCACCAGGAGCAGATCCCCTGGTGGAGAACTTTGAGCCT
CTCTATGACCTGGATGACTCTTGGGAAAATGCAGGAGAGGATGAAGGAGTTGTGCCTGGAACCACGCCTCTAGATATGGCCACCAGCTGGCAGG
TATTTGACATATTAAATGGGAAACCATATGAGCCAGAGTTTACATCTGATGATTTACTAGCACAAGGAGACATGAAACACCTGGCTGAACATGT
GAAGCTGCAGCTGTATAAGTTACTAGAAATTCCTGATCCAGACAAAAACTGGGCTACTCTGGCGCAGAAATTAGGTCTGGGGATACTTAATAAT
GCCTTCCGGCTGAGTCCTGCTCCTTCCAAAACACTTATGGACAACTATGAGGTCTCTGGGGGTACAGTCAGAGAGCTGGTGGAGGCCCTGAGAC
AAATGGGCTACACCGAAGCAATTGAAGTGATCCACGCAGCCTCCAGCCCAGTGAAGACCACCTCTCAGGCCCACTCGCTGCCTCTCTCGCCTGC
CTCCACAAGGCACCAAATAGACGAGCTCCGAGACAGTGACAGTGTCTGCGACACGGGCGTGGAGACATCCTTCCGCAAACTCAGCTTTACCGAG
TCTCTGACCAGTGGTGCCTCACTGCTAACTCTCAACAAAATGCCCCATGATTATGGGCAGGAAGGACCTCTAGAAGGCAAAATTTAG

TABLE 21
MOUSE NOMENCLATURE
ICSGNM Fyn
Celeram CG1380
HUMAN NOMENCLATURE
HGNC FYN
Celera hCG348O6
MOUSE SEQUENCE—GENOMIC
CCAACACGCAGTCCCAGTGGTTGGGAGACACAGGGCTCCCTGTCATTCTTCAGAGGAGACGCCTGCCTCACTTCAGACTTGCAATGCAGATGGC
GGCTGGCAGATTCTGCTGTGGCTGTTTCCACAACCGTGCTTCCGAGCAAAGGGGCAAGCGATGGGGATGCCACGCCCACTGTCCCAAGCTTGCT
CCTTTCGATGCGCTTTTCATGTTTTGTTTTGTTTTTTTTTTTTTTTATTACACTTTTACTTAATCTGTGTGTGTGCCCGGGCAAAGCTTCCATG
CAAGGCGTGGCATGGAAGAGTGGAGGTCAGACAACTTGGGAGCCATCAGTTCTCCCCTCTAGCAGGAATCAAACTCAGACCTGTGGGCTTTGCA
GTAGGCATCCTTACTCACTGAGCCATCCCACCCTCCGGCCATCTGCTTCTGTGAGAGCTGGATCTTCGTGACTAGTGCTACTGAATTAAAGGAA
GAGAAAGAACCCCCTGTTTTCTGAAAGGCTGGGGCTCATGCAGAATCATCTGTCCAGGAACCCCCATGCTGACTGGGGGGATCTGCGGAGTGAG
GTTTTGTCCTGCCTGAGAAGGAACCTCCTGGGCTCTCATCCTCCTCCTACCTATAGAAAGGACATTGGCGATGCCTGTTCAGGCAGCCGCTTTG
TTCTAGTGTGTTTGCTTGTGCCTATGCCAGGTGACACTGTGACTCCTGGCCCACCGCCAGTCCCGCTACAACTGTTACTAAAACAGAGGAGGCC
AGAGAGCCAGGCTTTGCGACTGAGCCGCAGATGTGACACTGGCTTTCCTGCTTGTGCTAGTTTGCGACTAGAAGCCCGTGGTTCTACAGAGCAT
GTGCAGAGTGGGGGAAACAAGCCTGACTGGATATGGGCAGTGAGAAGTGATGTATCCAGCAGACCTTAGTGTTTCAGAAGAGTGCTAAGTTGTA
TAAAGTGGGGCTGCTCCCTCCTGTGCTCCACCGGGATTCAGGAAAACTAGCTCAAAAGTCAAAGCTAGCTGGGCAGTGGTGGTGCAAGCCTTTA
ATCCCAACACTTGGGAGGCAGAGGTAGGTGGATTTCTGAGTTCGAAGCCAGCCTGGTGTACAGAGTGAGTTCCAGGGCTACACAGAGAAACCCT
GTCTCAAAAAAAAAAAAAAAAAAAAAAAGAAAAAGAAAAAAGAAAAGTCAAAGCTGTGTCAGCTGGAAAGGGTGAAAACAACAAGCATAAGGAC
AAATCCCCCTGCCTCTACAACCAGCAGCATCCAGAGAGTGAGTGCTGGTTAGACCTCCCTTCCCTAGGGCTGGCCAGTACACAGCTCAGCCCAG
TCCAAGCCCTCAGTTCCAGGAAGAGCCTGCTTAATTAAGGATTGTCATTCCTACATGGTTAGGGTCTCCAAGCATATAGGACTTAGAACTGAAG
CATATTCATTGCCCCCACCTTGTCTGCCTGTAAGACTCTGGGGAGCCTTAGCTTACCTGGGACCCCCTGAGTGAACCACGTGAAGCCGGGATCG
ATGCAAAAAGCAAGAGGAATTTATTGCTCCAGTGCACTGGGGCCATCCCAGACCCGAAGGAGTGGTGGCTACTCCCAATATCCCAGCGAGTTCT
TATATGGTTTCCAGGGGCAGAATAGAGCATCAGCAACTAGGCACAATAAGATTGGTGGAATAGTGCACCCTTTAAACTGATTGGTCTTTAGGGA
ATGAGGTGACAAAGACTTTCCTTGTCTGAAGGTGGCAACAGTCAGTACTGTGGAATGTGTCCCCACCGGCAGGTCCGATCCTGCCATGTGGTCT
GAGAAATGGTAATTAGCCTCTCCCTTCCGGAGGGTCAGGTGTTTCATGACCTTCCCAAAGTTCCTAAGCTGACCTTTTCATGCCCAGGTCTGGC
GTGATCACCCATTGCGTCCTCAGGTTTGACATCAGTGCCTCTGCTTCCCTTGCCACAGTAAGGTTTCTGCAGCTCTCTCTGTGTGTTCTCACTG
CCTGCTTGCTGCACAACCCCTCAGCACCCAGCAAGGAGTAGAAAGCACGCTCACTGCTCTTGACTGGTTAGCTATTGTGCCTTCCAGAAGTTGC
CTCTTAATGTGCTCCCCTCCCATCTCCAGTTTTATGCTTTATTACTATGTGATTGTTTCAACTTACATACAAATTCAGTTGAGAACTGAAACAA
GGGAACTTGAACTCATCAACATGGTGCATTCTCACAGTGCAGTCTCTGTCACGCGCCTGCGGTGATTCAGCCTGTCCCCCTAAAGCGCATCGCA
GGCATCCATTTTGTAGCTGGAATGGCCCAGTGCTGCCTTGACACAGCGCACTGCTTCAGAGTACTCACCGTGTCTGTTTTCTCCTCTGCTTACC
ATTCTGTCGTAGACATACCTTATAAATTCACCACCTCTCATTAAACACTTGCATGTTTTCTAATTTCCAAAGTGATGAACAGCATTTTATAAAA
CCTCACTTAGCACATACTCACAGGCCCAAAGGCAAGCCTGTCTTAGGCCCCTTGTTGACATTACCAGTTTGGTTTACCAGAAGTCCCCACAGTA
CTGTAGGAGCAAACTCATTTCAAGGGACCCTCTGTCACTGTTGGGTAGAGGATTCATCTGTGAATTTAGTTACTAATTAAATCACTTACTAATC
TAAGTGTTTCCCTGATTACTTGTGGAAACTGGTGTTTCCCCTAAAGCTTCAGTGGTGTGGGGGGAGAGGAAGGTGCTACCAATAGTGTTCCCTC
ATTTCCTGCCTAGTTGGTTTGTCTTATCTATATAAATCCATTTTTTAAAATATCTATCTACAAAATAAACTTGTACATAAATGTGGATAACAGA
ATTATTCAAAGGAGGAAGTATCCAGGATATTCATGATTTCTAAATGGATGAGCACCTTATGATATAGTCATACAGTGACAGATATTTAGTGATT
AATGTAATGATAACTTTAATACAATGCTATATTACTACACTGCAGATGAATCTTAAATGCGTTGTGCCCTGATGAAAAATAGAACGAAACCAAG
GTTGTGTATCATGTGATTAAATCCATACCAAATATCTGGATTAGGCAAACCCACAGAGACCTAGGTAGGTTAGATGGGCGTCAGGGAAGATAGG
GGTCATTGGCAAGTGGCTGCTTAGAGCACAGGGGTTTCTTTGAGGATGAAGAATGTGTTCTGGAATTAAATGGCAGTAATGGCTCCTTTATAAA
AACCAACTGTGCCACACTCTTCTGTGGTCTGGAAGAGTTGGGGAAGGTGTGCCCTGCCTCTCCAGAATCCCTTCGTACAAACAGGGCTAATACT
CTCATTTGCATCAGCACCTTCTCTCTCTCTTCTGTCGACTCAGAGATCCAAAGCAAACTGGGCGCAAGAAGCATCTCTCCCTCTCCCTGGAGCC
CTTCCATGATTCCCTGTTAAAACTTCATTTGGCCATTTAGGTTTGGGAGCCCATCCAGTGGCTGGCTGGCTGACAGCAGTGTGAATGCTCACTC
TCGGGGGCTCACGCTTCTGTCTCCCTTCCATTAAGCCTTCAGAGCTCCAGGCGCAGTGAGGGGGGTGGGGTGGGAAAAAGGAAAAGACTGCAGT
TTCTCAGCCTAGGGGTTATCTACACAAGCATCGACAGAGCTGATTCCAGGTGCCGGGAATGCGCCTCTTTTCTGCTGTGCCATGGTGTTCTATG
AGTGATTGGTTTCATCCATTGCTGTCAGGGTGGGACAGGATCCATCCTCGTCCCCTCAGAGCCTTTGCAGTGACCACACACATGGATGATCCAG
ACACTTGCTGTTTGTGAAGTAAAGCTTTGATTTGATGCGTAGCAGGTCCAGTCTTCTGGCCACTGCATGTGGTCAGTGTCTGCGTTGTGCACAC
TCCCCTGTCCCGTGGCCTTCTCACCTGAGAGACTGCAGGGTGTCAGAAGGAGCGTTTGGTAGGGGCTTTCCTTCACAGCATTCTACCACAAGTA
CATACGTCTTTTGTGTTTGGTTGGGGGTGGGGCAGATTATACGAACACTTCACTTCATGAGTTAATTTGCCTGTAAAGTGAATCATGTCTTAAA
TGCAATAGGAGAGCTCATTTTTATACCAAGGAAACTTTCCGCTACCGCCTCCCTCGTGGTATGTATAAATACCCTTGCCACAGATAGGGAAGAC
GGGGAAATTGAGGGCTGCTTTAGTTTTGTTGGCTCAAACAGTCATTAAGATGCCAAACCATACCAAGGAAGAGGTGACTCACTGTGGTTCCAGC
ACCTCCATAGAACCCACTATTCACAGTGTGGAATGTAGATGGAGACAGAGGCACCAAGGCTAACAGAGGCACTGTGCTCTCTGTCCCTGGACGT
GGTGTCAAGAGGCGTCCTTCCCTCTGACTTTCCCGCCATGCCTCTTTTGTACACTTTTGTGTGGACTTACCAGGGCTCCATGGTACAGAAAGCA
GCTTTGTTGCAGAAGTCCTGAGTTCTGGACATGGACTCTAGTCACCTGTTGTCACCTCTCGTCCCCACCCCGAGCTGAGCTGTGGACACATTCC
CCTACCTCTCTATAACAGGGCCCATGTTTGAGGACTTAGAATGTGGTGGTGGGGTTGCGTTTTCCAGTGCAGTACAGTGCAGTGCCTGGCATAC
ATGAGTATGCATACATACACTTCGGATGTTGGCTATGACTGCCAGGATGTGGAACCACCACCTCTACAGTGCCCCATCCCGTAACTAGGCATTC
ATCTTCTGCAGGCTTCGGACAGCATCCGTGTATTAGCTCACCATTCTGTGGGCCGTAAGCCGGCACGTAGCATGACCGGAATCTCTGATCAGCG
AGGCTGAACTAAGGGTGTCAGCTGAGCTGTCACTGGGGCCTTGCTAGGGAAGAATCGGTTTCCTGACTCACACAGACTCTTGATCAGTCTCAGT
TGCTCTGGTTTGCTGTGGTCTTGGAACTGACATGCCAGTTCCCTGCTCTGCCAGCTGTGAGCGCTCCTCAGCAGTGGAGAGCCCACATCTGGCC
ACGTGTTAAGAGCTGCAGGAGAGAGGATCCTCACTTTGAATGCTTCTTCTCACACTTCGGCTCTGCATGTGCAAGAAGAGCCATGACCTCCAGG
GCTTACTTGATGAGGTCAGCTCACGGGGACACTGTCCTTCCTAAGGTTGGCTACGCCAAGTGACACAGCCTAACACAGGAGCGGCTGTTTAATT
CACACATCCTGGAGACTGTACAGGAAGTTGGAAGGTAGCTTACAGTTCTGCGCACCACAGCACAACCTCAGCAGGGCCAGGGACAAATGGCTCA
CTTCTTCCATCCATCTGGCAGCAAGTATACTTTGCCAAGTGGGCAGAACTGCACTGCACTGCGCTGACACAGGGTGGACCCAGCACTAAGGCTG
TCTGCGGCTCAGCCCCCAAGGTCTTACAGTTGCACAATGCAAACCCGGCTGGATCTTTCCTTTGGCTTAGGACCTCCACCAGTGCTTATGCAAC
CAAGAGGCACCAAGGCACCCTGCTTTTTCTCCATCCACCTAATTCCTAGGACCTCTCTGACTGTACCGAAAAGACAGATAAAGACCCTTAAGCT
GCCAGGGTCATCTGGCTGTGTTCTAGGAAGAAGGAACAACCCATTGGGCAGGATGGAAAGGTGTTGGTGAATCAGGAAACTTGGCTGGCAGGTT
CTGTAACCCAGCTACCACTGCTCCGGGCATAGTGTTGAGCTGCCTAAATGAGGCTCTAGCAGTTATGTGCGGAAAACTCCTAATCAGCTGCCCT
GGCTCCTCAGGGGATTCACTGTCTGCCGAGAATCTAGTAAAGTTCCTTAAAGTCCTAGTAAAGTTACTCATCCTGTGGATGGTGTTGATTTTCT
CAGCATCAAAAGCTGTGTGCCAGGAGCCTCTCTCCTAAGGTCGCCTCACTCTGACTGTGCTCCGCCTCCTCCTGCCCTCTCTCATCCCACTGCT
GTTCATTCATGAAGCGGTCCACTTTTGCCTCTGCCCCTGACACAGTGTGAGGAGCTGCAGGGGACCTACAACTCTGCTCTGAATTCCTCATTGC
TTATGTGTCGTTGGTCCGGCTCCCATCCTTTTCTTACTCTCCATTGTCAGCTTTGCCCAAGAAAGAGCGATGCTAGAGTGGAAGGGACCATGAG
GCAAGTCGTGTCAGAGTTCTGTGGCCAGACCTCACTGTCCTTTCCCAGGCCAGCGCTGCCCACTAGGCTCCCAGGGTGAGAATGGGGCATGGGA
GGGGGATAGGGATGAAGAGCTAAGTCTAGAGCTGTCACTTCTCCCATTCCTTCTTAAGTTGGCTCCAAGGTAGCAGCCTACTCTGCTAAGATGG
CGGCTACTCATTGGCTCTTTTCCCTGTATGTTGCAGAGAAAGGCATTTGCCAGCATGCCCGGTGTCTCATCCAGGCCTTGTTACCCAGCAGCTG
TAGAACTTGATCATGGGCATTGCTCTTGGGTTGGCAGCTGAGGAAATGGAAGCTTTCTCATTGTTTGTGTGCCCAGCCTCAGAAGTTGAGAGTG
GGTACCCACCTGGGCCCACCTCTGGTCAGAACGTACCTGACCTGTGATAGCTGAGCCCCCCCCCCATACTGCAGGGCCCCCTCTGTCACAGAAG
AACTGTGTGGAGGGTTTGAGGCCACACCATGACCGTAGTATAAAGCGATCATATCTGCTGTGCTCATGGGAGGAGGGTGTGGCTGTGTTTCAGT
GTGTCTGGCAACTCTTCCCCTGTTGTGTAACTTAAATAGATTTGTGGGACTATGTCCATTTAAGGAAACAGGGTCCCTTAGCTTTCAAAGGGAT
GGAGAAACAGGCTATAATCTGGATATCTGGACTGGTCGCACAAGGTCAGGTTAGTGAAGACGCTCTGCTGCCAGCTCTTCTAGACAGACACATT
GACTAGGTACATGGATGTGGTGTCTTACGAGTCAGCACTCCAGGGCCCCAGTGTTGTCAGAGTGGCACTGACAGTGCTTTGCAGTGACCTACAA
ATCAAAAGCAACTAGAATAGTTAGTACACTTACTCCATGTTGGTGGACACAGAAAACACCGAATAATAGAGATATTCTTTTCTGGTTTTTTTTG
TTTGTTTTTTGTTTTTTGTTTTTGTTTATCGAGACAGGGTTTCTCTGAATAGCCCTGGCTGTCCTGGAACTCTCTTTGTAGACCAAGCTGGCCT
TGAACTCAGAAATCCACCTGCATCTGCCTCCCAAGTGCTAGGATTGCCGTGCACTACCACCGCCCGGCAGAGAGATTCTTATCTTAGTCCAAAT
GCCCAGAAGTTTGCCTCACAGTGAGGAGCCTGATGACACAGAATGCCATCTGTATAGTGTCTCCTCACACTCTGTCTAGGAGTAGTTAGGCTGC
CTGGGCCACATGGGAGGGTGAAGACCACAGATTCTCACACAGATCATCATGATGGACTCTTAGTGTCTGCATTATTTCTGTTTTGTCTTGCTCC
CATTTCTCTCGTTACTCCTGATTCTTCCTCTAAATCAGTCAAGAATAGAGCGAGATCCGAGTAGAAGCATATAGTTAACTCAGAACATCAAGGG
CATGCACTGCGTTCCCTCTGCAAAGATTACCTTCTCCAAAATTGTTTTTTGATCATTTTGGTTTAGTCACTTGATGTCTGTAGCATGCTTAGCT
CTGCACCAGGCTTGTAAGGTGCACACAGCTGGGAAGCAGAGGACCAGCAGCCAGGCCCTGACTTCAAGGGGATTATGCCTTTCATACCTTCACT
GTGAATTCTGTGTTAACACTGTGGAACTCTTTTTAGGGACTTTAGAATGTTGCCTTTTCTTTGTTACGTGAGACACGATATCCTGTTTTCTGCA
AGAGTTCTAATGAGGTGGCTTTGTTGCATTAAAAAGATGGTTTGCAGTGAAAGTTCTCATTGGTTTTAGGTTTCGGAGAAAATCAGGGTTTCTC
CCGGGTAATACCTGTGACACTGGGAATTGCTTGTTGCAGGAGACTATTCTGTGCCTTGTAAGAAGTTTGACAGAGTACAAGTACCCCTTTCCCT
AAGTTTTGGCAACTGAAAACATCTGCAAGCATTGCCTACTATGTCTCCTGGTGGACAGGGGTGCCAGGTGTGAGCGTGACCCTCTAATTCTAGG
CTCACATCTTCTGCTTAAACAAATCAATGAAATCCCCAAGAAAAAAAGCACTTGGAGCAAGTATGTTCTGTCTGGTCCTTGCCAAAGCTCAAAG
CAACGTTGTACCATGGAAGAGTTAAGACAAAGTCAGCGAGGTACATACCCATCTGATGGTGCAGCAGTGGTGGTGAAATGCCTCAGTAGATTAT
GCCTTGGCGATAGTTTCCTGTTGGAAACTCATTAAAATCCTTCGCTGTCCATAAACAAGAGAACGCCTTGGACACAGCATGCTGTAAATTCATC
TTGAATGATTTATGAGGAAATGGGTTCCAGATTTGGTGATCTCAGCTCAAGTAGTAGGCGACTGCCTGACAAACATTCTCATTCCCAAGAACTT
GAAGTCAACTCACTAAGCATTTGCTGAAGTGTAGGTTGTGAGAGCTCTGGAGTGTCTAATGTGTCCTGGTAAGACGAGCGCAGTGCTTAAGAAC
TGCCACCAGCATCTCCTCAGCCTGTGACAGCTCCTACGTGGATATGAGTTACCCTTTATGGTCTTGAATCTCTTCCTGGTCTGTGCCAGCCCTG
CCTTCCCCAGAGAGTGTATGCTATTAGACATCATGTCACGTGGTTCTTATTTGACGCTTTGGTCCCAAAGCGACTTTTCAGAGTCTTGGCAGGG
ACACTTCCTGCAAGTTCTGTCTGCCCACAGTGCACTTACAGTGGTAACATTCATTACCTTCCCTGTATGTCTGAGTTCTCACTTCTCACCTACA
TACCAGTAACATTAGAAAATGCAGGACAGTATGGTGATGCACAGCTTCCAGGTCATCCTAGTCTACATGGACAGCCAGTTACATAGTGAGACCC
TGTCCCCAAAAAGGGGGGATGGGGTCGAGGGAAGCAGGACATATTGTTGATGAAAGTCACTGGCAGAAAGGTTGGGTTTGGTACAGAGAGAAAG
AAAAAGAAATACAGCACAAAACAGACTTTTTCCTCATCCGGTTGTCACTTCTTCAGTAACTCCTGTGTGCTGGGCATTGGCCGTATATCTGTGA
AGAAATGGACCTTTGTCCATGGGGCAGAGGGACAGTAGACAGGCCACCTCTTAATGTGGCACAGTGGGTCCCAAGCTGAAGGAAAGCATAGAAG
CCAGTGGGCACTGGGACATGGTGGGTTCCTTGCTTGTCCGGAAGAAAGGGCAGATGTGGTCAAGGCCTCATCCCCAGCAGGAAGGTAAGAGCTG
ACGCAGGAAGAGTGAAGTAAACAGAGCAGCCCTGAGGAGGAGGCCCCTCAGGATTCGGATAGGAGCAGCTGGATCTTTGGGGATTTACCAGATG
AGGAGCCCTGGGAAGGCAGGAGAGAGGAGGGTGGGGCGGCATAGCTCAGCCTTTCCGTCCACCACTGCCTCTGGACCATAGCAACACTCTCTCT
AAATGAGCATTTAAGAATGTCAAACTCTAATAGAAAATTGAAATGCTAATTAGCAGCGTCAAGTGCTTGTGCTTCTGGAGGCTGTCATGACAGC
CTCTGGTCCTGCCCAGCTTTACTTTCACGCAGTCACTACTTCTCAGTTTTAATGCGGACCATCCTTTTGTGAGTAGTAAGTGCTCACAGTGGGA
GTGTCTGGTAAATAGTCCTGGGGCTGCATCCTCTCCTGTCCAGGTAACCCAGCATCCTCCTTAGAGGGCACTGTATTGGAAGGTTTTGCTCTAG
TGTTGAAAAACTCACTTTATTGCTCATATTAGTCCTTGCGGTGACTAAAGAAAAACCTAAGAAATTGGTGGCCAACTTTGGGCAGTGGTTGGCC
GAGGACTGAGATACCTCCTCATCCTTGCCCCTTGGCCCTGCTTTGATCAGCCTGATGCATCTCCTGCCCGTCTTCCGTGTTAGAGCAGCCAGGA
AACCTGCATGCTCACAGCCTCCTTTCTCTTCCATCAGGAGCTTGGATAATGGGCTGTGTGCAATGTAAGGATAAAGAAGCAGCGAAACTGACAG
AGGAGAGGGACGGCAGCCTGAACCAGAGCTCTGGGTACCGCTATGGCACAGACCCCACCCCTCAGCACTACCCCAGCTTCGGCGTGACCTCCAT
CCCGAACTACAACAACTTCCACGCAGCTGGGGGCCAGGGACTCACCGTCTTTGGGGGTGTGAACTCCTCCTCTCACACTGGGACCCTACGCACG
AGAGGAGGGACAGGTAAGCCATTGTCAATCAGGATGGGTGAGGGCACAGCTGTGCTCTTTCACAGTCATTGCTGTGTGTGGATGGGGTGGGTGC
AAGCATGCCACAGGTGNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNACACACACACACACACACACACACATACATATACATATACATGCTTCAGTTGACTCATTTTAACAGAGAAGTTT
TGGTCAACTTTAGGCCCAAGTTGACTATCCAAGAACCAAATGCCTGGCTTCTCAGGAGCAGGAGACCTCTTGTCACTGCTCTGAGAATTCAGCA
CCAGTTAGTGCTGCTAACTGCACTGCAGTGTCCACTGTGGCCTGTCAGCCAAGGGCTCCCAGTCCTGCCTACCCTGAATAGGGCCAGCTCTGGG
GGTACCCAAGCACTCCAGTGTGATGTACCCTTCCTGACTTACGGGGTAGTTTCTGTCCCTCTTCCCACTCTTCGGCCTGCCTGGCCAGTGTGGG
TGGTTGTCAGCATGTTCCTCATTTCCGTCTGCTTGTCAGTATTCCAGCCTGGACAGCCCTCGAGTTTTCATTCATCTCTATATTCTATTGTTAC
ATTCTCTGTGTGGGTTGTTTTGCTTTGTGTGACTTTTGAGACAGGCTGCCATTTTGTAGCCCAGGCTGTCCTCAGACTGTTAATAATCCCTCTG
CCTTAGTGAGCGTTGGGAGTACAGCCATGAAATATCACACCGGCTGTGTGAGTTTCAAGCTGAGCTCTGTGTGCCAAAACCATTTCATATTCTC
TCTCGTCCCTTCCTCCCCCAGGGCTCCTTCTGTTTCCCACCACAGGGCTGGGTTTCACCCGCACATCTATTTGCTGACTTAGCTAGTGCTCTAT
AACGTATCTGCCATACATCCTCACACACATTCCCTACAGTCTTTGTAAGCTGCCTCGTGTATTTGTAGTCTTCCAAATACTGTCCCAGAGTGTG
AACCATGGTAACCAGATATTTTGCATTATTTGTTCCAAAATTGTTTATTACAAAAGTTTAAAAGAATGTAGCGTCTCTTGGCTCATCTGGGGGC
AGATGCCTCGTGTCTGCTCTGACAACCTATCATGGCTTTTTACCCGGAGAGCTGACGCTGCCGAGGCTAGGGCTCTTTCTCTGTGGACAGCTTA
TGCAGTGGAGTTGGAAGAGGGTCCTGACCGGCCTTTGGACACGTGTCTCTTCCAGCACTGCACTGTCGATGTCTACCCTGTAGAACAAACCCTA
TGCTCACCCCTGTGCTGTAATGACACCAGCTTACCCTCAAATCCAGCCTTGTATGAGGCCCTAACCAAACCTCCATGGCCTGTACCTGCCCCCC
ACCTCAGGTGCCTGGAAAGGAAACAGACAAACAGTCTAGGGCCCTCTGTCCTTAGAAAATACAGAAGAAGAAGGAAAGCCAAATGAATACTCAA
CACACATGCACCTCCACTGTGAGAGGTGTTTGTTGAGATTTTTATGTTTGCTGACTCTTAATTAAGCAAACAGAAGCATCAACCCTGTTCATTC
ATTCATGAAGGCTGGATGTGAATCCCAAGTGAACATGTCTCATTCATTAAGGTCACCAGCATGGGCTCCTTGGATAGACTTAATGGAAGAGGCC
ATCTGACTCCTTATATTATTTGAAGTGTACACCGCCAGTACCCTGGGGCTGGAGAGGTGCCCTGGCTTAGGGGTTAAGAGCCCTTACTGCTCTG
ACAGATGACCCAGGTTCTCTTCCCAGAACCTCCATCAATGGCTAATAACCACTGGCAACTCCAGTTCAGAGGAAGCCAGTGTCCTCTTCTGGCC
CCTACAGGCTCTGGCCCACATATGGTGCACATAGGCAAAAGCACATATGTTTAAAGATATATTTTCAAGAGGCAGAGCCTGGGATATGGGCGGG
CGTTCCATCTTTGGGGAGGGGGAGTGGTTGCATTGTGTGTCCTGGGCTTTCTGGATGCCTTGCTGTCAGACCCCTAAGGTCGTGAGCAGTCTTC
ATTGCTTCCTCCTCCATCCCCAGGGCATAAAACCCCCCAGTGATGGAGAAAGAGTGCAGGAAGGGCTGTTTTCCACGAGTGAAGATGGCTTGGA
TCTGCTCTTATAAAAGAAGAGACATCTGTGAATGAAGTACGGAATCAGAGTAGGCATGGCCGTAGAGGCTCCCACAGTGTGTTCTAAGCCAGAC
TCCCCTAATCTCAGAGAAACCTTAAGAAAGGAAGACTGAAGGAGCCCAGCTCCCCGGTCAGGACATGCACCTGAGACAGATGAAAGGCAGGTCC
TCCCTGGAAAGGATGATCCCCACAGCAGTGTGGCCTTGGGAGAGGGGAAGCCAGGTGGTGATAAGCCATCACACTGGGAAATGTGCCGAAATTA
AAACAAGAAGCCAAATTACAGAGAAATTCAAAGCTCCCATTAGGTATCACATCCTGTGTAACAACCACAAAATTAACAGCTTAAACAGCACATA
CATTTATGATCTATATCTATTACCTGCGTTCTGTGGGTCTGAATCTGGAAGGGAGAGCCCGGTTAGAGGCTGTAGGCATGAGTGGCAGAATCAG
GTCCCTGGCCAGGGTTAGCCAGTCTGTCATCATGCTGGCGTGACACCCTCCTTAAGGTAGTGTGGAAGTAGAGAAGTCACCTTCTTCTGAAACA
TGTGTCACACTGGGGACATCTGTCCCCTTTGTACACTGTGTTAAGTAAATCAAGTTGTTATGTTCCTAAGGAAGAGTGTGCTGAACAAGGGCAT
GAGAACCAGGCAGGAGTCACTGTGCTCTGTCTACTGCATGGGGCAAGCCCTAGACTGAGTGGAATGCTGGGCTGATTTTTGCTGTTGGAACTTC
TGGGATGTGGGACAGTCCATGGGAGTCGGCAGGTGAGAGAACCTAAGATGGACGTGAGGATGAGGGAGTAGCCAGAGTGAGAGAAGTAAATGCA
AGGCAGAGGAAGAACCAAGGGGGCAGGAAGTAGCAACAGCAAGACCCCGGAACTGACAACCATGACCACAGCTGCGGGTGGGGGCCAAGGGCAC
TGCATCTCCCACCTGGGGTGAGCATTGCAGAGACGACTCGATGTCCCCGAGCCACAGAACAAGTCTCTTCCATTCACTGCTACCTTGTGCTAGG
GATGGAAGAGTGATCCATTTACCCAGACCTTAGCTCAACCTTCAGGGAAACACGTGGGGGATAAAGTAGGCCAGTACTTGCCACAGATTGATGT
CAGTCAGAGCCTTGTCTCTTACTCAGTGCTCATCCAGAGTCAACGTCCGTCAGCTCTAGGGTTTTGTCATAAAGCCTCCTCTATACAGACCACA
TTATAAAGCCAGCCATGTGACTATGACCATATTAATTACAATGCCTCAATATGTTTATTAATGCTAAGTGTAGATTTTTAAGGATGAGACTTTA
ACAAAAATGTGATTTCTTAGTATCAATTCCCTGTGTAATAGTCTCCGCTCTTTTTTTTGGCAGGAGTGACACTGTTTGTGGCGCTTTATGACTA
TGAAGCACGGACGGAAGATGACCTGAGTTTTCACAAAGGAGAAAAATTTCAAATATTGAACAGCTCGTAAGTTTGGGGTGGAGCACACATGCTC
TTTGGTTTGGGGGAAAGGTTCTCACAGCAAATGCATGTGCTATCTGAAAGTAAGGAAAGAATGCCTTCTGCACTCACTAGCTCATGGCATTCGA
GCCTGAGCGAACTGAGGACAAAGTGCAATACTACATGGTACTAAGTGCTAAAGAGGGCACCAGAGTCCTTTTAGAGCCTACTTCTTAAGTGTGC
ACGCGAGAGAGCAAGCTTCTGTGTGTGTGTGTGTGTGTGTTGCAAGGTCAGAGGAAGCAATCGGGTGTCCACCTCCGTCACTTTGAGGCAGCGT
CTCTCCTTGATCCTGGAGCACCTAGGTTTCAGCTCGGCTGGCAGCCAGCAAGCTTAGCAGTGGCCTCCCCCCCCCTCCCCCGCCCCACACCCCC
CTCGGCCAGCACTGCAGTTACAGGCTTGTGAGAGACACGCTGCTGGGATGCAGACTTGTGAGCGAGCACTCTTAACCACTGAGCATCTCTCTAG
CATCTCTCTAACCCCAGGGTCCGTGCGTTTGCATCTCAGTAAAGAACATAAAGCAGCCGTGCCCATCCCCGGGAAAAGCCGCATTCTCAGATGA
CTGGCGCACGGCTTGGGTGCCAGCCACGTGCTCTCTGCTCTGAGCTGCTCTCCATTATTCAGGTGTGGGCATTGTCAGAGGCCTCGCCCTTCAG
GCCAGCAACCAGATCCCCCCATCTTCACAACTGTGAAGCCTGTTGAGGTGGTTTCCTGAGGGAAAATATGGGAGTGTCTGTGCTCTTAAGAAGA
AAGCAGAGGCCTTTCACCGACTTGAGAATGTGACCACCAGGAACAACTGTCACCAGGGAGCAGGCAAACTTAGGGTAAAGGTTAACACCCTTTT
CAAGCCCCACAGTTAGCACCTGTGGAGGAAAAGCAGTTGTGCCTCTGCTTTCTGTCAGTGCCAGAGCATCACCTCCCTCAGCATGCATAGATGA
GAGAACGGAAGGAAAATCCACTCCCAAGGAGATGGTACTGTCGTGCCTCAGCACCTCAAGTGTGTTGATGAGTGCTCCCCACTCATGGGCAAAG
ACTGCCACCTGGGACGAGGCACCTGGGGTGAGGTGGGGTGGGCGCTGCTCTTGCTAAGAAGTCTGATTGCGATTTCTCTGGCTGAATTTCAAGG
CAGTTGAATGGCTTGAAGCTTGGCCCTTCTGCCCCCCCCCCAAAAAAAAAGAGGAACCTAGGGGTTGAGGTAAGAGGTAACACATACTGGAAAG
AGGAGGGTGTGGCAATGGAGAACCCCCTGTAACCTCAAGAGTGTCTCCATGGGAGGCCTGCCCTTCTCCAGGGTGCCCCTGGCTCTCAGCACTG
CACGGAGAACATTTTCAACAGCAAGACCCCATGCCTCCAGACACACTAGTAACTCTTCTAAATGCCAGCAGGCACTGTCTGCATAAAGCAGGAG
GCTTAAAATTAGGAGCAGTTGTCAACACCAGAAGAGAGGTGGCTGTGTTTTGGTGTTTTAATTTATTGAAATGCATGGCAAAGGTGTGTATAAC
CACGCCTGGTGCTGGTTGGGCAGCAGTGTATATGCTGACTTACTGTTTCGTAACCTCACATTTAAAAATACAAAAAGTGATACATTGATGTTAC
ATGTATTTTACCACAAATTTCAAGGACAGAATTTACTGTATTCTCACATACTAAGCATGTCCGTGCAGCCAGAATTACCTCCCCCTCCACCCCC
AGAGAGCTCCTGCAGGCACAGGGGTTTTTAAAATAGTAAATGGCTGCCATTTATTGAAAGTGCCGTATGCACTGACCATGTCATTGCTGTCGCT
GATCCTTCACAACAGCTGTCTGAAGTATGTGCCGTGACATCTGAGAGCACGGGTTAGGGAGCTGCCCAGTGCACCGTCCTGGCAGGTGGCAAAG
CCCAGGGTGAGATGTGGGGCTGAGAATTCAGAGTGTGGTGTCAGGTGTGACCTGGGAAAAGCCCAGATGCTGCCATCAAAGGCAGCTGGAGTGT
CTTAAGTGACGGTGGGAATGGAGGGAGGTGGCTGCAGCGTTGGAGAGAGGGGCGTTTGCCTTTGATCTCTTCCCGTTTATGATTGCTTATTGTT
AGTTTGAGTCTCAGATGAGCGCTGCTTGTCAGAGTCTATTGACTGGGTTTTGTGTAAAGCTCCCCAGTGGGTACAATGTCCTTTAACTGACAAG
GTCACTAGAAGTTTGAAACTCTGTAGGCCCTAGGAAGTAAGAAGTAGAGGGGTAGAAAAAGGCACATACAATTCTGGGCACCTGTGCACACTCC
CGACATAGGCCTGTTTTGAGGTTGCAGCCATGGCCCAGTTAGTCACTGGCAAAGTCAAGTCCCTGTCATTGTCCTTTGTTATGGAGGAGTCTAC
ACAGTCAACATGGAGGAGTCTACACAGTCAATAAAGCGGATTTTAGAGGAATGTTTAGGGGATGCAGCCTATCTCACTGTGCATTTTATTTGAC
TGCATAGCTTTTAACGGTTGCAGAGTTCTCTGGTCCTCTGACCCTGTTCTCCTAGAAGAGAACATTGCTAAGGGACTTTTCCTTTAAGGATAAA
ATTCTTTGTCCTACATGGTATTTAAGTGCCCCTTTTCACGGTTATGGAAAAGAAAAGTAAGGTTTGTACAGCAAAACGATATCCTGGAGTCAAG
GGTAATAACAGGGAAGGGAATACAGAGCAAAAGACAGAGCAGATGGTGTGAAGGCCTCCCTCCCTCCCTCCACCACCACCAGCCCTGACAGGAG
AGGAATGGGGCTCTGTCCTCCCCGGGGCAGCTGCCCCCCCATGTGTTCCTTCTGACTACCCCTCCCATCAGGACGTGCTCAGAAGGCTCCTCCC
AGTGCACACAGGGTTGGAGGGGTCCTCGCTGACCCGTTGGGAGTTCTCAGTAGTAAAAGGCTTGCGTTCACAGCACTGTAAGTTCACCTTCTCT
CCCCACCCCCGTTCTCACACTTTGTAACTGGTGATTTACCCACCTTGAAACAGCATGTCCTTAAAATGCAGTGGGTGTTTTACATAGGTGCAAT
CATGGCCTGACCCAGGACCTACTGAAAGCTAGTTTCTGTCATAATCAGGAAAAAAAAAAATTGGTTTGATTTTTTTTTTTTTTTTAATAAGTTT
GGTGGTTTGGTTTGGTGTTCAAAACAGGGTTTCTCTGTGTAGTCTTGGCTGACCTGGAACTCACTCTGTAGACAAGGCTTGTGTCCAACCCAGA
GGTCCTCCTGTCTCTGCCTCCCCAGTGCTTGAGATTAAAGGCATGTGCCACCACCACCCAGCTGGGGAAAAAAAAAGTTTGTATTACCCAGACA
TGTGATAAAAGTAGTTTCAAGAGTCTTATCTTTGAGTAAGAAGTACAGCTGTGGAATGCAGGAAGAATCCAGTATCCCTGGCTGGCCAGACAGC
TCAAGGCTGGCTGCTCTTGGCAAAGCCACTTCCTGCCTCCATCTGATCTCCAAAGTATCTGGACTTCCAGCACCTGAAGCCGCAGCCCTGTGAG
GGACTTGAGGATTAAAGACGGGATGGAAGAACAGAGTCTGTTACAAAAACTTAGAAGCCATTCTCAACCAGTGTCGGATTAAATCACGGGTTTG
GGTCAGACAATAAGACAGTATCTTACCAGGTACGAGTGAATTAAAAAGCTGAACTGTAAGAACAGTTTAAAGAACTGCAGCTTTATAGGCTGGA
GAGACAGCTCAGCAGCTCAGGCCAGGGAACCTACTACAGTTCCCAGCAGCTCAGGCCTGGGAATCTAGTCCAGACCCCAGCAGTCAGTCCTAGG
AACCTTGTCTAGTCCGCAGAAAGCTGAGTGTGGCAGGCAGCACTACTTGTAACCCATTCCTGGGAAAGCAGACAGACAGGAGGAGTCACTGCTC
ACCCAGTCTCGCTAAATGAGCTAGCTGCAGGTTCAGTGAGAGTGAGAGTCCCTTTCTCAAAAACAAAGAAGAGAATAACTGAGAAAGATGCCCA
TGTAGTCCTCTGGCCTCTGTAGACATGCAAACACACTGGCAAGAATACCTGAACAGCATGCATCACACACACACACACACACACACACACACAC
ACACACACACACACACACACACACACACACACAGGCACAGATTTTCAAATGCTGATTAACTACAGTAGCTCAAGTCAAGAATAAGGGTCCTGGG
CTGGAGAGACGGCTCAACCGTTAAAGGCTAGGCTCACAACCAAAAATATAAGTGTAAGGGTCCTGTTAGAGATCCCGCTCCAACCAACACTGT
GTGCAGGACTGTGCAGGTGAATGGGTGCTGCCAGTGAATTAAATAGTTTTTGCTTGTTTATTTACCTAACAGCTTGTAGTAGATATTTTGAGTC
TAGGACCAGTGAGAATAGAGTGTGAGAAAAATGCCACAAGATGTGACATTTGCCTTATAATGTGTGACTGTTTGTCATTTGTGTGTCACTCTTG
TGAGGGGGAAAAATGGCCCACAAACGTTAAGTCCAGAGTGGTGCTATCTTTCTTCTGAACAACTGTTACATTCTAATGTGTGTAAACTTATTTA
GATGGCTACACCAAAACAAGACAGCTGATACTAACTTAAATTGGAAATTGCCTCATTAGTGAGTCAATTCATGATTACTCAGTCACCTGAGTAA
GACTGGGCTTGTCCCCGATTGCAGCAAGATCCCCCAACATCCTGACATCACCTGAAATTGGTGCTCGCTCTTTTTCACTTTGATTTAACTACAA
GTAACTTGATGAAGGTTAGCATGCGTTAGGAGACAAGCCTGGGCTGCCAGCTCTTCATTCTTCTTCACTGTCTGGAGCGGTTCCCAGGCTGGGA
GCAGTGAAGGTTGGTTGCTGTGAAGAGCCAGTGGGTGATCTTTATGTTTTTCAATGATGAAATGTCTCCATCTAGTGGAATTTCAGAGCAGTAA
ACACTCACTATTAAAATGAAGGTAAAGCCTTTTTCTTAAACACATGGTGGTCAGTATAACTTCCAATATGCATTTAGATGCTAGACTTACAACT
ACATATATTTCTGTGGGGCTAGAAATGGGAACAGTGCTGACTTAAAATGTTAACTTGGTTTTCTCCTTGGGGGTGGGGATGGGCTCCTGGCCTT
GTGTGCACTAGGCAGGCACGCGGCCACAAAGCATCTACGTCATGAGATGTACAAGGCAGAGGCACACATGCACACATGCACGAATGGTAGCTAA
GAGAGATGTTCACTTGACAAAGGACATGGCTCTCCTTGGGGGAGTTTTGTCTATGTAAATTCTTTGATGTGTATAAATATTTCAAATAAGAATA
TGGACACAATGTGATTATAATTAAACAGTTTTCTGGGTAAGAGAACTACAGTATCACTCTCATGGTTGATCTGTTAGTTTAATAAGGTTAAAAA
GGAAAGATGCTAGGAGTTTCTTTGTTTGTTTGTTGTCTCACTATGTAGCCCTGACTGTCCTGGATCCCACTTTGTAGACCAGGCTGGCTTTGAA
CTCATTGAGATTCACCTGGCTCTATTTCCAAGTGCTTGGACTAAATGTGTGCTCTAACACCCCTAACCAAGAAATAGGTTTTATAATCCCTCAA
AATTAGAGAGATAGCTCAGGAGTTAAGAGTGTGCTGCCAGCACCCACATCAATAAGCTCATAACCATCTGTAGCTCCAGCAGGATAGAGGTCTC
TGGTATCCAAGGGCACCTCCACTCCTCTGCACATTACTGCATAGAGAGGCACACATGCCTACACATAATTTTTTAATATAATTTTTTAAAACTT
ATATTAGCACTTTGGGTGTGTGTGTGTGTATGTGTGTGTTTTCTACTGTTATTTATCTCATAGCCACACCCACTTGGATGAGTTCCCATTTTAT
TCTTTTATTCTGTGACTTTCTAAAGCCCCACCAGATGTCACTGGTCCTTGGTACCCCTGCTCAGAAGTTTTTTCTGTAGGGAACCTGAAGGAGG
GAGGACTCTGGCTTAACAGGAGGCCAGGGTGTCTACCTAATGTTGACCTGGGGAAGGTCTTCCCAGCCCAAAATCAGTACGTTTTCTTCCCTGA
CTGAGCATCAACAGAGCCCAGCCAAAGACAATTTTAAAAAAAAGAAAAAGAAAAGAAAGGCCTGAGGTTTCTCTTGTTACCTTCACAGACTAAA
AAAAGAAGAAAAGGAGGTGATGTGTCTGTAATGGTCAGAGGCTGAGGCGCCTAATATGGTGACCGGCAGGCCTGTTTCCCTGGCCCTCATAGAT
AGAGAGGCTTTCATTTGGTGCAAGTCACAGCTAAACTCCACGCACATGATCATTGGCCCACCCACTGTTAATGGGAGGAAGCTCACATGCCATA
AACCTTCATGAATCCAAGCGTATGGATAATAGTTCTATATATAGTGACTGTAAACTGAGAGTCGGAAGTGCCCTTCCTGATTCACACTCTGAGA
ATTTTGCTGCAGTGGCTCAGAGATCTCTTCTGCCCTCCAGCTACACAACTGCCTGCCCTCCATCTGAATCTGGGGGTGGGGGTGGGGGGAGCTA
TGAATGTTTAATAGAGTAACATCCTGTTGAGAGTGTCTGAGGAAATCCTGTTATGTGGTGTTCTTATACTACCTGCAGAAGGTCATGTCTTCTT
CCTACTTCGTCAAGAGCAAGCTTGATAGGTCCACTAAGCATACATCCAACCCTCACTGGGCCTCCTACTGCTTCTCCAAAGTAAACTGAGGCTG
GTGTGTGTACTCCACACACACACACACACACACACACACACACACACCCCTCATTCAAGATTGCCTCCTACCCAGAAAGATGCTCCTCCCCGAG
CCCTAAGTGGGAAAGTTTCCGTTTCAGGATCTCCTGATGATGCCCTTGAACTCCTCAACCTCCTACTGGGATTTCCACCCTTAGCTTATGTGGG
GCCGGGTATTGAACCTAGAGCCTCGTGCACGTAAGAAAAGCACTCTGACTTCAGCGCGTGGTGCTTTGGATTTCTTAGGGCCCGTGTTGAAAGT
TTGCAGAAGGGGGCACAAGGCTACAGAAAAGGCATAGGGTCGGCCTGCAGAATTCCCAGCTGGATGGTGCGTTCAATTCATGTCCCTTCTTTTC
CTTTGCAGGGAAGGAGACTGGTGGGAAGCCCGCTCCTTGACAACCGGGGAAACTGGTTACATTCCCAGCAATTACGTGGCTCCAGTTGACTCCA
TCCAGGCAGAAGAGTATGTTGTTGTTGTTGTTGTTGTCTGTCTAAATATCTGCTTTTAATTGTCACATAAAATGCCAGTGACACAGCACAGGAG
CGAGAAGACCTTTCCCAGTGGTTAAGGGTTGAGAGGCTACAGTCCTGCTGTGCTGTCGAGTCTGCGCAGTACACTGTTATTCACTTCGTGGTTT
CCTTTGAGCCCGTCCGTTTTCTGATTGAGAAGCTCTATGTGAGTACCCTAGATGAAAACCCAGTAACTTGCTCCAGATGAAAAACCAATGTGGG
GAAACCACAGCTGCAGTGTGTGTTACTGAGAGCCCACCGTGCGCCTGCAGTCTGCTCCCTGAGCACTTGGGAGGCAGATAATTATGAGAGATTA
AGCCCACACACACACACAGGAGTCCTCTGCTTGATAATCAGAAATACTGAGCCTTCTGAAAAGACATAGCTTTTTGTTCCCCAGGCAGAAGAGC
AACTTGGAAACTACTGCAGTTGTGGGCTTCGAGGGATATTTTCCTTGGACTGAGGTCTCGGTTGGTGTTGTGGCTGGTCGACCTTGACCAGCAG
TTATTTGGCCCCACCCTCCCACTGGAGGGAGGCAGTTTTGGAAAGTGGTAGGAGTCTGGGGCTCTCAAAGGATGGCTGACTGGTCATTGTTCAT
TCTCTTTCCAGAGAAACCTGTAGGAACTCTTAGGAAATGGGGAGGGAGGTTGATGTCCCAAGCTCAGGAACATGGATGGCCTCTACCCCACCTA
CAGGGACAGACCTCACCAGAAAGAGAAAGGAGTCACTGTCGGTTGCTCACGGCCTGCAGCCTGCTGTGGATTTGCGGAGGCACAGCACAGAGGA
GTAGACCTTCACCCTAGCCTAATCCTCATGCGTCCATCATTCTTCTTTAAGTCTTTTGTACTCCTACACTTCGATCTGTTTCTGCTTCTCCAGC
ACTGTGTTTTGAGCTGTTTTACCCCCTTCGTTACTAGTGTGTTTCATTATCAGAAAATAGGAAATATGTGTATTTGTATTGAATTTTTAGGACA
GATTGGTAAATATCTCCTGTGTCTTTTCGGAATTTAAAACTTAACTACAAAGTAACATTCCTTTGATTTAACTGAGAATCCACTTGGGGGTGCA
TATTTAAGTCATATTTATAATGATAAGGTTTTTTTTTAATATTTTGGGCTTTTTAGAGTATAAGTTAGCTTTTTAAAATACAGCCTGTGGTTTT
TACACGTGCAATTAATCGGTATTTTGCACAAGCAGCGTGTGGTTAAGTGTAACCTCTTGTTCTGCAGGCCTTCCTCTCACCAGTTTTACACAGC
ATTTTAATTGAGTTACTGATACAAACCAAAAAAAGACATTTCTCACATAGCTGGTGTCGTATCCTTTACAGCAGCTATTGCTACGCTGGGATTG
TGGAGAGTTTGGGTACTTCTCAAGTCACTGAAGGTGACGCTCCTTCGGTGAAATTCTTTGACTCACAGAAGGATCTGATGGTGAACTTCCTAGC
TACATTCAGGCATGGTGAATTGAAGCTTCATTTTTAAAACCATTTGCTTTCATAGATGAATACTGTGATGGTTTGGTCCTGAAAGCTGCTCCTA
CTCACAGATTCTGGTGGAATGTTCCAGAACTCTCCCTTGGGCTCTTGGCATACTTATCCTCTCATATACAATCCAGTGCTGAAATGTGAACGGA
TTGCCGGGCGTTGGTTAGGACTCAGCAGCTCAAGTGGAGAGACAAAAGACGCATTCCGGTCCTGAGGAGGCCTGTGAGGCTTCCTGCCACGGCA
CAAGTGTGCTGTCAGTGCTGTTGCAAGGGAATCTTAAAATGTCTGCACCACCGACTCATTTTTATGGACATGCCCATTAACCGTGCACAGCTTT
CTCTCAGATGTGCAGTAAGCAATCCCTGCAGCTGTGTCAGAGAAGCGCAACTGCAGGGCACAGGCCAGGTACTTGGTAAACGCTCCTCACGAAT
GCTGGACAGGGAGAGAAAATCAAACCAGGCTTCCAAGAACAGCAGCCATCTCAGATCAGGGGTAGAGCTGAGTTCCCGTCTTCAGAATTCCTGT
AGGTAGAGACTAAGTAGAAGTGTTATCTGTCTTCCTTCTTTATCCATCCTGAGTGGGCTCCTTTAAAAAAAAAATCATGCTTGATTGATGATCC
TTATACTAGAGCTGAGTCTTGGTATTTCCCAGGAGTTGTAATTATGCTCAGAAATCAAGGGTCGTACAGCTGAGCTATGGAATCGTCCCCTGCA
GTTGGCATTTCAAACAAGGTGGTGGGTTTCTCTCTGTGCGTGCAGGTGGCATTTCACACACACATCCCTGTCGTCCTGGAGGGAAGGAACACAA
TCCATGATTCAAGATTTTTTTTTTTTTAAGAATGGAAGAGATGCAGAGTGGATAGAATCAGGAAAGATTGAAAAAGGACTGAATGAAGCTAGAA
AAAACTAGTTTGGGGCAGTGGAAGCAACATTAGAGGCGAAGGAATTTGTGGAGGAAAAGCTAGCTACATTGCTCAGTCACAAGTGTATTTATTT
TCTTGCCTGTGTCTCCCTTGAAGGTGGTACTTTGGAAAACTTGGCCGCAAAGATGCTGAGAGACAGCTCCTGTCCTTTGGAAACCCAAGAGGTA
CCTTTCTTATCCGCGAGAGCGAAACCACCAAAGGTAAGCTAAGGAGAGGGCAAAACTGATGTGCTGTTCACATCTGAAGGTCTTTGGGATCGGA
AGGCGTGGCTCTTGCCTGGTCCGGGGCCTTCTAGCCTGAGCCTCCTGCTGCTCTCCACCTCCCCTGAAGACGCTCGAACTGCATTTACCTCCCA
GAGATGATCGGGCTCAGAGGGAGGCGCCTCCAGAAACAGAGGCCAGGCTTCCGCCTCAGCTCAGACTCCGTCCTGTTCTCCTGTTCGAGTCAAG
GGCGCGAACTTGGTTTTTCTTACCTTTTTGATTTGGGGTCTGTGTGTGTGTGTGTCTGTGTGTGTGTGTATTTTCAAGACAGTGTTTCTCTGTG
TAGCCCTAACTGTCTTAGAACTCACACTGTAGACCAGGCTCGCCTTGAACTCAGAGATCCACCTGCCTCTGCCTCCCAAGTGCTAAAATCAAAG
GTCTGCCTGCCCTACCACCACCCAGTTTAAATTTGTTTTTCTTTCCTTCCACTCTGTTTTGTTAGTGGAGGACTCCTTCCTCTTCCCTCTGTTT
AATAACTCACTTCAGCAGGCAGAGGGGCAGAGAACCATGCTGCCACACACTGGTGACCTGTCCCTCTGCAGATTGCCAAGCAGAGCTTTGGAGC
TTCCTGCTAGGGGATAGACTTCCATTTCTTTTGTTCCCACCCTCTGTTCCTCCCGCACCCCTGCCTCCCATCAACCACCTCTGTCCCCACCCCT
GCCTCTCCACCAACCACCTCTGTCCCTACCCCTGCCTCTCCACCAACCACCTCTGCCCCCACCATCCCTGCCTCACACACACACACACACACAC
CCACAGCCCCACCACACACACACACACACATATGCCATCCCTGTCTCCCAGCCCCTCACTATCCCTGCCTCTCAGCTAGGCCACAGGAAATGAA
AGCAGGAGGCAAGCTCACCTGGAGTCTGCTCTTCATCTATCCCAGTGGGATTTGGTCTGTGCAATATTAGACAACACAAGAAACTGAAAGGAAA
CTTTGAGAAAAACCAGAAGTTTTTAGTTCCAAGAGCAAAGTCAACTGTATTGCTAACATCTCTGAACACTGGCAGGATAGCAAAAGTTGTCTTT
GTCCTGTTACAGAGTGGAGATATATCTGTCTCTTTCGTATGTGTGTGTGTGTGTGTGTGTGTGTGTGCGCGTGCGCATATGGTTTGTAATAATT
ATTAATCAGAATAGTCACATAAATGAGGCCTGCAGAGCAGACTGGGGCTCCGATGATTGGTGTGGCTAATGCTTGCACGTTTACCAGGTGCCTA
CTCACTTTCCATCCGTGATTGGGATGATATGAAAGGGGACCACGTCAAACATTATAAAATCCGCAAGCTTGACAATGGTGGATACTATATCACA
ACGCGGGCCCAGTTTGAAACACTTCAGCAACTGGTACAGCATTACTCAGGTAACCCCAGTGCCGTCACCGTCACCGTCACCGTCACCGGTTAGA
AACTGGAGGCTGTGAGGGAGGAGGAGGAGATACGGGACAGAGTCACTAGGTAGAGCTGTCCTCTGCCCGCCCACCCCCACAGTCAGACAGACAG
ATGGAGTCCGGGGTTCTGTTGCTCTTCTGCCCCAGATAGGATTTTCCTCCTGTGTTTTCCAGAAGTTCCCCCCAGTAGAAGGCAGTAGTTTAAA
TCTTGAAAGCTGTATTTGTATATTAATTTTCATAATAAAAATCCTTGCACGTTCTTTACCTTTGCCTTCATTGCCATGCAGAACTGTGAGAGGA
AAGGAGGAAAGGAGCTGTCCCTAGGCAGGCTCAGACGGCCCCCAGGGAGCCTCACTTGATCCTTCACCGCAGCAGAGGGAAGCACATTCTTGCA
CTCTGTAGAAGAAAATCTGTGCTGCTGCAGGGTTCCCCTCCGCCCCGCCCCCACTCCCCCCCCCCCCCCAGCTTGCCTGCTGAGGCCACAGCTC
CCTTACCTCCATCTCCTGGACTTTCTTGGTTTTTTGTTTGTTTGTTTGTTTGTTTTTGTTTTTTCAATTTTTAAAGACTGGGTCTCGCTATAGC
CCTGGTGGCCTCCAACTCAGAAATCCCCCTGCCTCTGCCTCCTACTGCTGAGACTGAAGGTGTGCGCCACCATACCCGGCTACTGTGATGACTT
TAAACAAAGATTAGACTTTGTCTTTACGTCCCATAATCCTGTTCTTCTGCTGTTCTTGGCCATTCAGTTATTGGGTGTATGTGCGTTCATGTGC
GTCTTATGTAAATATGTAGCTCTATGCGCAGTGTTTAGTGTGTGCCGCTTGCCCTCCTTGGTTAACAAGTGTGTCCTGCTAAACAATGCATGTG
TACATGACCCCTCCTTTTTTCCAAGGTAAGGTCTCACTTGTAGCCCTAGGGAGCCCAGTATCCACTTTGTAGCCCAGGGTGGACTTTCAGAGTT
TGAGCAATCCTCTAGTGTCAGCATCCCAAATGCTGGGATTACGGACGGAGCCACCACACCCTTTTTACCACCATTGAAAAATACCTGTGCTGTT
TCTGCCACACCAAAGAGACTTGTAGCCTTTTAAAAATAACAATACGAAATGCCTATGTACTGATTCTGGCCACTGATGAGCAGTTAAGAAATTA
ACAGTGGCTGAGCCAGTGATTTGGGCATTAGCTCTCTCACTCTATGTGGATTCTAATGAAATACTGAGAGAAATGGAGCAGAAAACCTCCTTGC
ACTTCAGTGATATACACAAAACGCCTGTATGTTATAAGGATTTTTTTCAAATGCTGAAATATATTTCATTACAGGCAAACATCCGCAGGGTCCC
CAGGGTTTGGATCATTTGAAAATAGTGTGCTGGAAAGGGCACCTTAATAACATCATAAAGCTGAAGGAGGGAGCCGATGTGAAGCACCCTAGAC
GAGTTTTAATCTCAGGGAAAAAAATCCATCTTGAGTTAGATTTGTTTTTATATTGGCTTTTCCTCTTTCCTCTTCTCTCTTGCACATAGAATGC
CTTTGAAATACCGACACATAAATAATTTCCTGCCTAAACATCTCAGAAACAGATTTAAAAAAAAAAAAAAAGGAATGAAAAGTAAGTTAAGAAT
TCCCAGAAAAGAGACAGCCACAGTTTGTGCTTAGAATCCAAGAGGCTTCCAGGGACGTAGGGGGCTTTGGCCCCCTCGGTTCAGGGTCTCTCTG
GCCCCCATCTTTCCCTGCCTGCTTACTCTCTGTTGGCGTTTAGTCAGTCTCCCCATACCTTTGCTAGATTCTCAAAGGAATATTCTTTTCAAAA
AGAAGGGGTGGGGGAAGACCAAGTTCCCAGTAACCTGAATGGAAGCTACTGTTTCCCTGCTTTTCATGTCACTGAGTTTCTGTGAATGTTGTTA
ATTTCCATTACTTCTTTAAATCCAGACTCCTAAATTATGCATAGAAAGCTGTTTAAAAGGTCGTGTTTTTGTTTTTGTTTGCTTTTAGTGGAAA
ATTGCAAAAAAGCAATGTGAGCACACAGGAGCAGTAGAGTCAGAAGGACTGCAGAGAATGTGACATCCTCTCCCGCTGCAGGTGTCCTCCTGAG
ACCGGCTAGCTCTAACAGACCAGAGGTCTGTGGGAGGCTCCTATGCCAAAGTCACATTTTAAACAGACATTTACCAGGGAGAGTCCCTCAGTCC
CTAGATACCGGAAGGGACAGGGACAGTTTTTATTTTTATATGTACTTTTTGAAAGTAGCAAGGGCATTTTGAATTTGAGGCAGTGAATACGAGT
TCATTAGACTAAAGTACACAGAAGCCAAAGCAGGCCGTGCCTGTGAGCAGCGAGCATGCCCGATGGTACAGCGGTGTCCATATCTGACACATCC
TCATCTGCCTCCAGCAAATGTTTGCTCATGTGCGTCGAATCTCCTGTGGCCTGTGCGTGTTGTCGTTACATATTCTGCTCTTCTCTCCCTCACC
GCTGCCCTGCTCCTCCAGAGAGAGCCGCAGGTCTCTGCTGCCGCCTAGTAGTTCCCTGTCACAAAGGGATGCCAAGGCTTACCGATCTGTCTGT
CAAAACCAAAGATGTCTGGGAAATCCCTCGAGAATCCCTGCAGTTGATCAAGAGACTGGGAAATGGGCAGTTTGGGGAAGTATGGATGGGTATG
CTGAGATTCAATTACTCTCTTATTAGCTTCCTCGTTTGGAAAATCCCAAACATCAAGATGGAAGGTGGAAATAAAGATATTTTATGATCAGGAC
AAACAGACAGTTTGAATTATTCAACTTTTATTTGGAAAAATTTGGAATCTCTCAAAGAGAGCTAGAAACAGGCAAGAACTACAGCTGTCTCCAG
TTGTGCTACAGTGTTCCAACCTGGCTCAATGTTGCCGTCAGGGAAGTCACCTTCCATCTTTCCTAGTGCATGCATGATTATGGATTATAAGGAC
TGTTTTCTTTATTAATTCCCTTTCCTGTAGAGAAAGCTGATGGTTTGTGTTTTAACTTAACTGTGGTTTCATCAAGTTGTACCCCACAAACTTC
TGGATTGGCTAAAGATGCTTGGGAAGTTGCACGTGACTCGTTGTTTCTGGAGAAGAAGCTGGGGCAGGGGTGTTTCGCTGAAGTGTGGCTTGGT
AAGGCAGAGGGCACATTCTGTTTTGGCTGCATCTCAAAGCCAATGAAAAGGACAGTTTTGAACAGAAATGTGTATGTGGCCGTTGGGACAAAGA
CCCTTAACCTTTGTGACTAGATGGGCTCAAGCCACCCAGGTTTGCAGAAGTCAGTGGCACCTCGCTGTGTTCACCTGTCCTATTTCATGTGGCT
CATTAAACACCTGCAGGACAGGAACCCAGTGATGGTGACGCTTTCTCTTCCTGTGAAATGGGATGGTAAGATGCCATAGAGTAAGGTCCTGGGC
TTTCTTCACACCAAGCCTGGGTCTAAACCCTTGCTGGGTGATTAAGTATAAATGCAGGGCCTGTTTAACTCCTCTGGCTCCAGTCCTCATCTGT
AAACTTGGAATAGTACCTGCTTCGCTGGATTATTTAAGGATTATATCAGACATGGTTAAAATCTCCTGTAGATCTTGGATCCATGCACACATAT
CTTCATTGTGTGCACATTCTTCAGTAACGGCAACTGCACTGGGCCCTTGAGATGGCTTTCATAGCCCTCTGGGTCAGTTGCAACTTTACTTACA
GACAGACTATAAACTTAGACCCCTCCCCAAAATGTTTGTCCATAAAATGATAACAAAGTGTATTGGGGTCTCCCTTCCTTAGAGTCTACAAGAA
TACAACATGTACAGATGGTCGTAAGGTGTCAATCTTAAGCCATTACATAAGCACCAGGGTGTTTCCCACAGGACATCAGAGCTGGCGTAGGTGC
CTAGGCAGTTACCCAAGCTGTGAGGAGTGGAGATGGCACTATGCTTAAAGTTACAAAATGTTATCAGTCAGATCCCGTAGACCACCTAAATGAT
GTCAGAGTTCCCGTTACACCAAAAGGCAGACACTCTCTGAGATAGAAGAACTCTTTACACCTGCCACTGGTTCTTTTTCACTTGTTTTTCCCCT
GTAGGATGCATGTTGTAATTTTATGTTTTAACAGAATAATAAATGCTTCAGATATAGACCCCTTAGAGTGGAAAGTCATAGATGACACCTAGGT
AAATCTTAGTGTGAACCTGGATTCTCTTTCAAATTGTAAGCAATAGAATCTCAAATTAGATTTTTATTTTCCTTTTATAAAATTATCCTTCAAA
GCCATTGGTTAGAAATAGAACAAGCCAGAATTCTGTCTTTTTATTTGAGAACAGAAAGCCAAGCTAGTTCATTCCTGTTTTACTGATGAACTGA
CAGTGGCCATGAGCCAGGTCTGGCCTTTGGGTCAGCTTGATTTTTAAAGACGTACTTAAACATGTCACATGTACGTACTTTTCTTTCCTGGCTG
CTCTTTGATTTCCCAGCCCACCTGATAGAAGGCATTGTTCCTGTGGCTAACTCATGGGAAGCTGGAGAAATTGACCTTACCTGGGAAAGTATGG
GCAAGGAGATGGTGTGCTCCTGGCCTGTGCTTAAGGGTGCACTTAGCTTGTCTTCTGAAGGACTCAGTCAAGTGTCTCATCCAGTAGACAGTTG
AAAGCCTACTCCAAGCATCCTTGGTATGGCCAGATTTTTATAGCCTCTAAGATGGAATTTAATCTAATTTCTCTCAGGCTTTTCCTTTATCTAA
GGGACAGCTGTTGGATACTCAGGCCAATTCTAAATCCTAGACTGTCATGACTTAAATAAGAGCCCAGAGCTTTCTGGTGTAAATTCACAATTAT
ATGCATAAGCCATAAAAAGAGTGATACATATCAAATGGGAAAAGTTAGTGAATTATTAAACAGCTGTGAGTAAACTTGCTTTTCCTTAAATACT
TGACAGAGTCCTTGCCATTATCTTTGCTCGTAAGAACTACTTTAAGGTTTCAAAATTTAAAGTCTGATTTTATGCACAAAGTTGCTGGTCAAAA
TTATTTTTCCCATCAAAAGAGTACCTTTGGATTGACTTACTGTTGTGGGGTATCTGTAGGTACCTGGAATGGAAATACAAAAGTAGCCATAAAG
ACCCTTAAGCCAGGCACCATGTCTCCGGAGTCCTTCCTGGAGGAGGCGCAGATCATGAAGAAGCTGAAGCATGACAAGCTGGTGCAGCTCTACG
CGGTCGTGTCTGAGGAGCCCATTTACATCGTCACGGAGTACATGAGCAAAGGTCAGCCGCACCCCTCCCCCTCCCCTCCTCTCNNNNNNNNNNN
NNNNNNNNNTCCCTCCTCCCCTCCTCTCCCTCCTCCCCTCACAGGGCTGCCGGGGACCGGAATCCTCAGGGATTCCACTCTGCTCACTCAGGAT
AACACTTAGAAAAGGGGAACATGGAGTTTGCCCCTGGATTCCTGAGAAGTGCAGTGCCTTTTGCATGTTCCACATCAGGAACGTTCCTCTGCAC
AGTTAAAACATCCATCTTTCTATCCAAGCTAATGCAGAGCTGAGGCTGTGAGAGCTGCTGCCATTTTGAATGGGTGGCTTCTGTTCGGTTTCCC
TTTTTAGCGCTAAGTAATCAATAGCAGATGGTTGCTGAGTTAAGTTCTGTCCCCATCTCAACATCCTTCCCCCCAGCTTTCCCCTTAAACATCA
GGTGAAGAGGTGACACGCAGTCATGACCTCCCTCCCTCCCCTCTCGGCGCATGCATGTGTTGACGCACACGTGAGCATGCGTACATCATTATAA
TCAAACCGGCATTGTTGGGGAATGAAGCCAGAGATCATTTTGTTTTGGAAAAATAAATGAGCCAAACAAGAAACAGCCTTAAAAGGCTGTGTGC
ATTGTACATCAGAAGACACCAGGGTAATTTTTCTTCTCTGTGTACTTTCTTTTAAAATATAATTGGAATTTTTTCCTGGCAGATTATATGCCCT
TTGGATCCAGGGTTATTTTGTAAATAACTTTGTGATTAAAGCATTAAAAAAAAAAAAAAAAAAAAAAAAACACTGACAGCCATCCAGCCCCTGC
AAATGATTATCTCCGCCTCGGTGTCAGATCCGTGCCTCCTCACTCAGCCTCACACGAGTGGCTCTGAAGCTCTGGCTGTCCGAGGCACAGGCGT
GACTGGCTGGGATCTAATTAGGAAGCTCCTGCCCCACCCATATTCCTTGGATGCTTAAAATCTGAAAATAAGTCAGCAGTTCTGAAAGTCCCTT
GTACCTGAGGCACCTAGAGAGCTTCTAAAAATACGGGCATCTGTGTCTGACTCTGGGATTCCTGATGTAGTGGATAGAGCCTGAGGACACGCCC
TTCCAGCAAATACTGTCACTACTGTTGCTACTTGTCGGAGCCTCAGCCTGCTCACCTGAGCTGGCTCTCCACATCAGTCGCCTGTACGTCGTCG
GTTACTGGGACATGAAGGATGGTTTGGTTCCTCCTGGTGACAACATAAAAGGAGACGTTCATTTCTTCTGGTTTTACTAACTCCTTTCTCACTG
CTTATTTTCAGAAGCTACAACTCGCTTCTTAAAACATAAACTTGATTTCTTGGTTTTTGTTCAAAGCTTTCTTTTTCCATTGATGCAATTATCC
TTTTCATCTCCCTTTTACTTCCACTAAGGAAATCAAATACAGCCACTATTGAGAATCTTTGCCTCTCTGAGTAAACTAAAATTGTTTTGTTTCA
TTCATAGGAAGTTTGCTTGACTTCTTAAAAGATGGTGAAGGAAGAGCTCTGAAGTTGCCAAACCTTGTGGACATGGCGGCACAGGTGGGCCTGG
GGTCTTAATTCAGGCTGTGGCTGTACCAGCACGGACTCTCAAGGTGTTGTGGTGTAACAGCTGTGCCCCCTTCCCTGGGTTTCCACAGGTTGCT
GCAGGAATGGCTTACATCGAGCGCATGAATTATATCCACAGAGATCTGCGATCAGCAAACATTCTAGTGGGGAATGGACTAATTTGCAAGATTG
CTGACTTTGGATTGGCTCGGTTGATTGAAGACAATGAATACACAGCAAGACAAGGTGGGCACTTCCAGGGCAGTGGCTCCTGATTGATCTTTGC
CTGCACAGCCTTCTCCTTACCTTCATTTCCTCCCTCTTCCCTTTGTTTGTCTGATCTGGCGCGAGCTGCACAGTTCTCAGGTGGGAGCTAGTGG
ATCTAGTTGGCGTGTGCAGTGCTGGTCTAGTAAGTGGGCGTCTGTTCCCATGGCTTGCCTGGCTTTTGGGAAGTATCTAGGCCCACGGTGTTCC
ACTTTTGGAGAAAGAAGGCTTTGAGAAGCCAAGTTATAGCTCATTCTCCATTAGCCACCTCTGAGCAGAAATCAGGTGACACTCCAAGTCAGAC
AAGTTCCTTTACAAAGGAACAGGCGTGGTGTTGGAAAACGGAGAACATAATAAATAGCAGGGGAGCCAAGGCTCAGAATGCAACAGGAGTAGGT
AGCGCCAGAGCAAAGACACCTCCTGGTAGAGAGCCTGCACATGGAGGCGATTCCTGATCTCAGTCAGCAGCTGCCTGCCAGGCCCAGCCAGGAC
CAGCTGGGACCAGCCTGAGACTGAGCTCTGCAGAGCGCTGAGGAGAAAAGAAATGGTCTGGAGGGCCTGGACCACTGCAGTGCTGGAGAAGCCG
TCTCTTCTCCTGCCTCACCCACCCCCTGCAAAGGGTCAAAGACATTAGTTTTATGTAAACCACGTACCAGACCAGAAGCCAACCCACTTGGGGA
GGAGGGATTGTTTGTCAAGCACCAGATGTCAGGAAAGAATTCCCAAGGTGTTTTCTGCCTTCCTGGCTGGCTGCTTGCATGGCAGTGTCCAGGC
ATAGCACTGAGGAACTAGCAACAGTGGAGGCCTCTGTGTGGGTTTTAAAGCCGGAACTCAGGCAGGGAGTAGGTTTCCTTGGTGAAACTAACCG
AAGCCTGGCCACCCCAGCCAAGCTGAGCAGTAGTGGGGATGGATTACAGGATGGTGCATTCTGCAGTTTTATGTTGAATAGTAGTTGTTTCGCT
CATTTTTGGCAATTCCTTCCGCCTTTCACTTGCCTCAGAATGGAAGTGCAGTGAATGATGTGAGAAAATGTTAAACTGGGGCCCTGCCTAGGTA
CTGACACTATCCAAGTGCTCGGTTGCATGTCGGACAAACCGCCCAGCTATTTGAATTCCCCACTCCCACCCCTGGCACACGCCATCTCTTGCCC
TTGTGGGATCCTGGGGGGTACGGGGGGGGGGGGTATGCACACAGACACACACCCAAAGTCAGGGGTCACAAAAGCAGGCTGTATCTCTAAGCAG
AGAGGAATACAGACGTGACTTGGTGACAGGGCATGCCAAGAGGGTCTGAGTGAATATGATCAGAGGCCTTGCCCTGAGGGAGAGCCTGTGAGGG
TTGATAACCAGCATGTTCCATTTAGGAAATGGCTCCAAAGAAACTCAGGAGCTTCCGTCTGTGTGTGAGAAAGTCTCCGTGGCAGAGGGAGCAG
TGCAAGACTGTTTACTTTTAACTAGACTTTGAACTCTGTAGAGTCTATGGTTAACACCCTTGACCCATCTGTGAATGAGAGGTGTCAGGCCAGG
AGCTAACAGATGCTACCAGAGTACTGTTGGGATGGACTGGGTACATAAGTCATGCATCATTCATAAGGGTGCCTAAGACTGTGGCCTCAGGCGG
CTGGCACCTCTCATGTCAGGTTCTGCACTGGAACCCATCCAGGGCCCTCTCAGCCTGGCCATGCAGCCTTCCTTCCCACTTCCCACCATGACTG
CCTCTCTGCCCTGCCTCCCCCCCATGACATCCAGACCACAGTCTGAGGCCCTTTCCAAGGACAAGCCTCAGTCCTTTGTTCAGCATTTCCTTGA
CAGAAGAGTCCACCTTTCCCTCTTAGAATATTATTTCTTCTTTCTTATCTTTGTCAACTGTCTTCAGATGCATACCAAACACTGAAAAGGGTAA
GCAATAAGGGTTACAGGAGCTAGAGTATGACAGGAGAGACGGGCAGTCTGTGCTGTCCTGAAGCTGAGAACCAGGAAAGGCCAAGGCACGTCAG
GACCTGGAAGGGGCAGGAAATAGACCCATAGTTAGCATATTTGTTCATACAAATCACAAAGCTCACAAGTGGAAGGTCCGGATTTGAAAACAGT
ACAGCCCACCTGACCAGGTGTGCTCTTGAGCTGTGCCCACTGTCTCCCCCCAAGACAGTGAATCAGAGAATAGAATGGAAAACCAGAGAGAATT
AGTCCTAAGTCCCACTTCCCCTTTATTTTATGGAAAAAGCCTCAAGGGACAACCTCTCAGATTCACATGTGTCAGGGGACCAGAGAGACTTTTC
CTTTTGCAGCCTTGGCTAGCCCAGCTGTTCCTACAGTTCCTTCACTGTGCCTGCCCTGCCCTGGCCTGTCCACGAGATGGAAAGGTCCGGCATG
TGTTTGCCAATGTATTCTGCAGATGGCATGTGTGCTGCATGCCGGCGTTCCTGCCGGGGATTAATAAGCTCCTCTAGACAGCTGGTCCATGTGC
CTAATAATATTTGCAAGTCGGGTTTTATGTCACCCGCAGGCCACAGCCGTGCCGCGTGTTGATTATGGGAGTGCAGGCAGTGTTCTGAAACATG
TGCCAAATGTAGCTATTTTAGGACCATGAATGACAAATTGTTTGCAAAACAGTTGTGAGTTTCCCATCTCAAGGTGCTCTGAAATGTCTGTCAC
CACGGACAGACACACACACAAGGTGTGACAGCACAGGACACAAGGACACTGCAGTATGGAGCTCTGTTACAGGTCTTCCATGTGTCTTTGTCTC
CTACAAGCCCCATACTCACCCCCTGCCTGCCTGGCTTGTGAGGAGAAGCCCAGTGTCAAAGAAGAATTCAAGACCAGGCCTAGTGCCTATGAAG
GTTACCCTTCACTTGACAGCATTCTAGAAGCAACTAGAATCATTTGGGGTTCCTTGTAATGGCCTGGACACTTCAAAATAAGAACTAGTTCTTG
TTTAGTCATAAGCTCTGCACTAAATTCTGTCCATCTGGAGTCGCTTAAATAATCCATAGCAAGTGTTTGAATAGTTTGTTTATCAAAGGAACTT
GGCTAAAAGTTGTTAAGGAGATACCCAGGCCAAAGCAGAGGCCCTGTGTGGTCTCAGCTCCTCTGGGGACTGAAGAGGGAAGATCTCATCAGCC
CAGGAGTTCGAGACTGGCATGCACAAAACTGTAAGATTCCACGTCGAAGGAAGAAAGGAGGGAGGGGGGAACGCAGAGGAAGCCTAAGGGCACA
TAAGGACAGTTGGCTAGCATGAGAAAGAACACAAGAACATTAAACTCATGCAGCAAAGCCCAGCAATACCCACTGACTTGGGCCCTGGGCAAGC
TGGGTGATTCGTCCATGGGCAGATTACTTATTTTTAACTGCAAATGCATCTTCCCTGTCTGTGCGTGTTTGCAGCTACTGGAAAGCACTTGTGT
GTCTAACCTAGCCCCAGCGAGTAGGAACTGGACAGTGTCGCTTGCTGCTGTTCTGTTTTTACTATACAGAGGACCAGGCTGCTAGAGAATAAAC
AAATGGGTAGGTTGCAGTTGTACTGGAAAGAGCCAATTACCATTCAGGGTTAAAAAAGAAGCTAAAGGCCCTTTCCAGGAGGCAGGTGTAGGCT
ATGGTTAGAGTGGCACTGTCAGCAGCAGAGAAGGGAGAACGTCGGGGCCATCTTAACATCTCAGCGAGAGAGAATGAGTATGAGTTGGGATTTG
TTGTATTTGAGGTGTCTGTGCCGGTGCATCTAAGCCCAGAGAAGCTGCTGCAGCATCGCAGCTGATCCACTGCCACAGCAGGGTCCTCTGACCC
TCATCCTTCCCACTCTGTCACCTACGCCTCTCCTAGAACTCTACTGACAGCCACCTCTCTACCCCCAGTCCCACGATCCTTCCAGAAGTTACTG
GAGCCCCCAGCTGGGCCCAGGGCTTCGAGGTGAGGCTGTGCTCTTGAGCCACACCCTCTCCTCAGCATCAGTTGTTTGCTCCTCTGTCACAGCC
CCTGGCCATCCGTTCCTTCTCATGGAAAATGAAAATGGGTGGGAGAACTCTCCACAGACTTGGTCTTTTTCCTCATTCCTCAAGCAGCTGAGAG
CTGGCTAATGCTGATTTGAACCTGGCACACTCAGCTCAGCTAGACCTGGCCATGTTTAGCAATGACTTGCTCTTGTGGACACTTGATTTCTCTT
CTCTTCTCTTCTCTTTCTCTTATCTTCTCTTCTCTTCTCTTCTCTTTTTTTTTTCTATTTTCTTTTTTAAAATTTACATTCATCCAGTAAAGTA
GACAAATCTGGTATGCAACTGGGTATGTGTTTACATAGTTTTGAAAGCACCTCACAGAGAAAGACAGACCTATAAAGACCATTTCATTCCACTC
TAGTCCACAATCGGCCCCAGAGAGGGCTGTTTTCTGTTGCTTCAGATTCCCTCTCCCTGTAAGGAAGTTCATGAGTGTGACTTCACAGATCCCT
TCCTTAGTAAGCCACCACTCTCAGGAATAGCCTCGCCTAGCCGTGACGTACTCTTTGATCTGCTTCTTGCTTCCTGTAACAGGCCTTGGCTTCT
CACAGCAGACTGATATTTCACTGAGCAGAGGTACCATGTGCCCCATCACTGTACGGAGGAGCAGTAGGGCAAACTCTACTTGGGCTGGTACAGA
TGAAGCTGCCGGGCGGGAGACACACCCCTATCTCCCGGGTACTCACCCTGTCAGAAGGGCTGGTACTGGCTTGTCAGCAGCAGGTGCTGTCCCA
AGAACACCGATTTAAAAGGCAACTGCAGCTAGGTGACTTGCCTGGTTTGGAAATCAGAACCCCCAGGTTTAAACCACTTCTGTCACTGACACAC
AGGGTGGCCTCAAGCAAATCAGCTACCTCTTTGGAGCATCATGTTTATCCTCTGTAAAATAGATAGTAACAGGGCCTGCCTCACATGGATACTA
TATGATTGAGTAAGCAATCTATAAAAATTTGAGGGTGGTAAAATATGAAGAATTATTTTAAAACTTCAATTTTTTGTGATTGACATGTTTTCCA
TAGAGAGGGTTTTATGGTATTAAAAATCATTTAAAGAGAAAAAAATGTTAGTAATCAATAGAGGAAAGGTACTTATAAAGCCATAATACACAGC
CAGATAGGTGGATGATTGATTGATTGACTGATTGATAGATGATAGATAGGTGAGTGGATGTGTGTGGAATGTGTGCATATGAATGTGCATGTGT
ACAAGAGCACACACATATCATTATTTATAGCCCAGACCAGTCTATTTTTCTGGTTCTATCTACATGTCCCACAGACCCTCTGTTCTGAACTCAA
CGAAGGGAGAGTCAACTAACCCTGCCCCTCCATTGTCTTGGGCAGGCCTTCATTCTTTCCTCCTGTTAAGAACCATTCATGGCTGGGTGGGTAT
GGTGGTACACACCTTTAATCTCAGCACTAGGAAGTCAAAGGCAGGCAGATCTATTGAGTTTGAGGCCAGCCTGGTCTGAGTTCCAGGGCAACCA
GGGCTACATAGAGAGACTGCCCACTCCCACCCCCACCCTCACCCCCACCCCCAAAAAAGAAAGAAAAGAACTATCCACAGCCCCGAGCTGCATG
CACATGGTTTTTATTTATATTTATTATATATCATTATTGTGTCTGACATCACATATGTACATAAGTTCTCACATGAATCCTGGTAGCAATTCTC
CTGGATATCACTAACACAGGTCTCGCTGTTCCTCTGTGGGGATGGGGGCCTAAAGACTGAATACAGCCCTCATGAGCACGGAACATTTATTGGC
ACTGTCTGTGTACAGCAGTGGACATAGCTCTTATCACTGCCCTTCTCCTCGCTCTCTGCAGACACACTTTTCTGCTCTCTGTGCAGTATGTACA
GGCTTCCCAGAAATGGAGTGCTAGAATTCTGTAATCCTGCAAGAACCCTTAGAGAAGGTTGTTGCTTATGTCGGTGTTTGCTGCACACTGTGAG
CTGGTCTCTGGGGGAAGTTCAGGAGCTAGCTTCCCAGCTAGATAGACCTGTGCTCAGATGCTAGCCTAGCTCTGCCTTCCCTTGGGTTGCAGCC
TCTCTGGGCCTCAGCATAGTCATCTGGGACCCCCTCTCTGGTGGCTGTGGCTTAGAAAGGGCAGAGCTGTTTGGTGTCCTTTGCTTCTGTTCTC
CCTCACAGGACTTTCTCCTCCCAGACCTCCTACCTCCTTTCTCTGTATTCCTCACTCTGTTCAAAAAGATGGCCAGGCCTGGGCTGACTCTGAG
CCTGGCCGGAGAAGTCGTGGCATTATCTTAGTATGAGTCCTGGAATCTCTGCAGATCTGCTCGGGCACCTGGGATAAGTTGCTTTGAACCTGCC
TTCTTCTGCAAACAAAATTGTCACACGGTCTGAGAACAGACAGGAAGCACTCACCACAGAGGACTTAGTGCAGTTGGCAGTGGCATTAACAAAA
GCAGTGATAAGTTGACCCTATAGACACATACAGCCGTCCTCTGCATACACGCCCACAGATAGTACCCACGAGCCATGCTGTACAGGGCCTTCCC
ACTGCCTCCTGCCAGGCCTTGTCCCCATTTGAATCATGAAGAGCAGGAACATGGAGTTTCTCTGTGGGTTTGGTGGGTTGGTTGATTTGTTCCA
AGGCAGCAACCAAACGAGCATATAGGAAACACATGTCAAAACGATGTAATCTTTGAGTCTGTGGACTATTTGGACAAAAGGAGAACACTCAGAA
CTCTGGGTAACTTTGTTACTGGTTTATATATTAAGTAGAACAAGAAAAATGTGTCTGTTCAAATACTCCCAGTATTGAGCATTTAAATTGAATA
GATTATTGTCTAGAGTAAAATTCACTTATAAATTAGACTACAAAGAATTCATAATTGTTACAGTTTCCATGTTCATTCGTTCACGTTAAAAATT
GTTACTGCATAAATTAACACGTTCTTTCTTGGAGAACATATTTTAGAAAGATTTGTTACTTTAATAACTCTTTGTGTATTAGAAATCCCAGATG
CCGGTGCTAATTAAATGTAATCAGATTTTGGATTCAAAAAGCACAGTAGCCATTTAGCACAAAGGAGCAGGCAGCTAGGTTGACAAACACGGTC
AGGAAGGACTGGGTCCCTGATTCTTTTAGCTTTCCAGTTTCACAATGCAGAGCTGTGAATGCAGTTCCTGTCTTTCCTTTGTTCTGATGACTGG
GGATTATTAGACTCAGGCCGCCCCCCGCTGTTCCAGCTCAAGGACTCCTGCAGAATTATTAATCAGCTGCTTCAGAGGCCCTGTAATCCAGTGG
CAGATCAAGGCTGATCTCACCACGAGGTGCCCTCCCTCTGCTCCCCTCAGCGTATGAATTTCTTGTTCTCTGTCAACAACAGCATCGCCATTCC
CACACTGTGTGCTGTGACCTGAACAGCAAATGTCAGCAGAGAAGGGGGGAGGTGGAATCCTGTTAATTGTTGAATTTAATTTAATTCTTTCCTT
GCTAAAGAACTGAACCCATCTTTTAAGAAAAGCAAACCTCTTCCCGTCTCTTATTCCCCAGGCAGAGCTTTTAAAAGGCTGTAGTGAAGTGACC
AGGCCACACCACCCCACAGGCCCACACGGGGAGTTTATCATCCCCAGAGCTGCCAGAAGCCATGTGCTGAGCCCCACGTGCAGAACTGAGCCAA
GCTGCCTCCCTGAACTGTCCAGGGCCAGAGTTTGGTGCACCTCTGTCCCTGGGCAGAAGTCGGCTTCATTTGTATTCCGCTGCCTCTGTCTCTA
GGCTTCCTACCACTGGTGTCTCTGGGAGGGACAGCTTTGTTCATCTGCTGTAGCTTCCGCCTAGATTAGTAGTCAGGGGAGCAGGGCAGATGCT
GGGAAATGGAGATGAACTGTTGGGAGCATCATCCCCTCCTAGCAAGGCTGTGCTGACAGCCAGATGAAATAAACGAAGGGGGCTTTTAAATCAC
TCATTTCTCCCTAGTCCCTTTTAAGATTAAAAATGCTTGCAGGGTGGCAGGAAACTGATTTCTACCACTTCCATCCTTGGACAGCTCTGTGAGG
TGGCCTAGGGAATGCAGACAGGGCCTGGTGCCAGCACAGGTCCCTGAGCCGACTCTGAGCACCTTCCTCTGTCTGCCCAGCCCACCAGTAGTCC
TGCTTTGTGCTCACAGGGGCATCGTCCTCTCCTCTGTGCTGTTTTGGGCAGAGCTGCCTGGCAGTTGTTTATGAATGGTCAGGAGCAAGCCCTC
ACTTTGTACAGCCCACGTTTCTTGGTAATCGTCACTAAGGAAGCAGATGGTCACCCTCTGTGATTTTAAAGTGTTTAAATAGGAACTGCAGACT
GCCATCAAACATCAGAAACGTCTTCTGGCTTCCTTGTCGTACTCTGCTGGCTGGCAGAAGGAGCCTGCATTTGCATCAGCTGGAGAGTGAGGGA
CAGGCAGAGCACATGCACACTGCCTGCAGCTACGAGCCTTTCTCCTAACCAAAGGAAGGTACCTGGGCCCAACACACTGCTCTCCACAGGGTCC
CAGAAACATAGGCACTTTGCCAGATTGGAAAGCAGAAGACAGTTTAAACCAACCTGTGGTCATGCTTGTCTTCTGTACGTCCCCTCCTCTTGGG
ACGTTTTTTCTAGACTCAGAGCCAAGCCCCAGTATGATGAGCCTGTCCCACTCAGTATCCCCACTGTCCACACTGCCTAGCACAGTGTGGCCTG
GCGTCGCCCAGGTCCCTCTGGTGGGTGGCTCACTCTGTCTGTGTTCTGCTACATTCTTAACCGAGTAATGGGCCTGCCAAGAACCCTCTCTATC
CATGCTCACTGTTCAGCCCCCGGGGAGCTTGCTCACGCTTTCAGCTTGAACGTTTCTTCCATGGATTTATCCCAAACAGACCTTGGATCTGCGC
CTAGTTAATCAGCCCAGAGTTTTACATTATGTTCAGGCCCAACTCCTGTCTCCTGGCACTCAGGTTTCACTCATCTCTCCCCTCTCAGAGTCCC
TGATGAGACAGCACTGTCTTAATCCTTGTCACCTTCTCTGTCAGACTCCTTCCTCCACGCCCCATCCCCAGCCCCGTAAGCTAAAAATTTCAAA
TGGAATATCAATCATCCTTTCGTGCAGCTGGATTTTCCTTTATATTTATTCTCAAAACATGACGCTAAACTCTATGAACTTAGAGTGGAAATTT
CAGCAGAATATGTTACCAGGGGTAACTGGAAACACTCAGTTTATGATTTCAGAAGCATTTGGGAAATTTGAACGTCATCCTTGGCCTATTATGA
GAATTAGGAGTTTTCCTCTGCCAAATTTGACCGGGTTTGAAATTATTATTGTACTTGAATCACCACCAAATTCCAGGTTGGGTCAGCTTTGCTT
CAGCTCCTAACACAGGATATCCTGTCCTAGACTTCTTGCTGTCTCCACCATCCAGTCAGCTTACTGTTACTGCCATAGTCATGGCTGCTGTTCT
TCTCTAGGGTGACTGTACAGTCAAAGTACTACATACTGTTCAGCATTGTATAGGATAATTCGCACAGTCTGTGGCTTTGGAGTTAAAGAGGCTT
TGGGGGTGGCTCTTTGGTTTAGAAACCTAGGCTCTATTGTTGAAGCCAGTGCATTGGATGGCTTCCTCTGCTGCCCTGGTTCTTTGGATCGCTT
CCTCCGATGCCCTGCTTCTTTAGCTGGAGGAGATAATCTCTGAGACAGTGCAGTGGAACTGTCACCGACAGTCCCAGGCTGGACTGAGCTTGTG
GTGTGTGATGGCCCCAACAGGCTTGTATTGACCCCAGCTGTGATCCGTTCTGTGTCTGGGGAAGTCATGAGTCACACTGGTCCCTTCTCTGTCC
CCCATCTCCCTCATCTCCTCCATGGCCCGAGCCATCATCTTCGAGACAAACCACTTTGTAGCTCTGTAGGTTCAGCCCAGTAGCTGCATTCCTC
CAGCTTTAGAACATTCCTACAGGAGGCTCCCTCCTCCTGGCGACCCTGGAGTGCCCCACGCTGTGCTCAGGGACTCGGCGGCTCCCTCCTCCTG
GCGACCCTGGAGTGCCCCACGCTGTGCTCAGGGACTCAGTGGCTCCCTCCTCCTGGCGACCCTGGAGTGCCCCACGCTGTGCTCAGGGACTCGG
CGGAGGACACTTGGAATTCTCGGTGGCTGATGGATGAGTGTGGGGATTCTCTGCATCCTCTTTCCAGCTTTTGGAAGTTTCGAAGGGCAGTGAT
GCTGAGGGCCTTTGTCAGTGCAACTCCATCAAAGCATTTTCTCAGCTCTCAGCTTGTTACTTACTGGGCATCAGACACTCTGCTGAGACCTGTG
TACCTCCTGCCATCCCAAGTTACAGACAGGAAACTAACACTGGGGCTTTTTTTTTTTTTTTTTTTTTTGGTTTTTCAAGACAGGGTTTCTCTGT
GTAGCCCTGGCTGTCCTGGAACTCACTTTGTAGATCAGGCTGGCCTGGAACTCAGAAACCCGCCTGCCTCTGCCTCCCAAGTGCTGGGATTAAA
GGCATGCGCCACCACTGCCCGGCTGACAATTGGGCTTTTAAGGGTCTGGCAAACCCAGTCTGCAAGCCCAGGTTTGCCTCACTCCAAAACTCCT
TTTCTTCATTTATTTTAACGTCATTAAATTCCGTCTTTACCAGCTCCACTCGTATCTGCAGAGCCTGATTTCTACATGACCATTCTTGCCCTTT
GGAAGAACATTCTAGCCCAGCCCACCGTCGGGCTTGCTGCCACTGTCACTACAGAGAGGTGGTCCTTTTTCCTAGCTCTTCCTTTTACATCATT
CAGAAAGCAGCCTTGGGACAAAAGGTATCTGGAGTGTCCCTGTGAGCATTTCAAACATTGTCACCATCTTAACGTATGAAGGAGAGTCCGCTAC
AGGAAGCTACTAAGTCAAGACCCGTTTCTGTCTGAGGCAGAAGTAGAGGACAGGTTTCCTGGCTGACCATTGGGCATGCAGCCATTCCTTTAAC
TGGACTGTGACTTGGGCTTGGAGTGGCCCATCCTGATGTGACTCTAACCGGACATCGCGTCTTCACTTAAAGCAGAGAGCACTAGGCTGGGTAG
GCTGAGAAAAGCCATCATCTCTTACATTATTATATCGCTAGTATTTTCTTTGTAGTCAGTTTTTTTTTTGTTGTTGTTGTTTTGGGGTTTTTTT
TTGTTTTTTGTTTTTTTGGGTTTTTTGTTTTGTTTTGTTTTGTTTTTTTTAAATCTTGGTCAAAGAAGACAACATATTTGGCCATTGGGGATTG
AGTAAGTCACTGCTCAAGTGGGCCTTTGTTGTTTTGTTGTTGTCGCTGCTGCTGCTGAGCAGTTGTGGTTTAACAGCTTTAGCCAAAGTACTGG
GACAGTGTAAGTATAGAATACGCCAACCTTGAAGGTCTGCGCTGTGCTCGGAGCTACACTCAGCAGCATGGAGCACGGGGTCTAAGTGCTCTGA
CGCTGCAGCCAGTCTCCTTCCACACATCCCGTTTCCATGCCTTACACTTGTTCCTCGACGTGTTGTTCTCTCAGCTGAAGAACAGAAACGTCTG
CGTGTGTCAGATGTGCGGCACACAGAGCTAGTACCGATGATCTGTTCCATAATAGCAGGCGCTTGTAGTGTCCAGCATTAAATTGCCACTTCTG
ATCAGGTTCCTCCCAGAAGACTTCAGGAAGAAGTCTTGAGGAGACAGACAGAAGGCTGCAGCTAGGCAAGTGGAGGGAAAGATGGCATTTCAAA
CAAGCCGATACAACAGAAGGTCTGTGATCACGCCAGGTGTGGTACCTGCTACAGGAGCGAAACGCAAACTAACCTGGATTAGCTGTATGTTTGC
AATAGGAAAAACCGAGGCTGACTGGATATGTGCGCCCTGGAAACCAGGTGGTGGATTCTGATTTAATTCAGTTGGGACCCTCCCACATGGCTAC
AGAAGCTGCCATTTACAAGGATTAATTGGAGGGGCACTGCTGAAGGCTAGGTACCAAGGCAGGGGGACTCGGCAGGGGCACTGCTGAAGGCTAG
GTACCGAGGCAGGAGGACTCAGCGGGACTGGTGACTAGAATCTGGGTAATTTTGAGAGACAGGTCAAAGAGCAGAACCAAGGTGCCAGGTTAAC
AGCAAGAGGACAGTGGTCTGGAAAGAGCAAGGGGAAGAAAAGTCCCAGCTTAAGGTTTCTGCCCTGAAGCCTAGCAACACAGTGACCAAACCCC
AGAAAGGGATAGTAAAACACAAACAAATGAAGGGATTTTGTTCCTTTCTTAGATGTAAAAAGTAAGAATTAATTTCTTTTTTTCCATTTTTTAT
TAGATACTTTCTTTATTTACATTTCAAGTGTTATCCCCTTTCCTGGTTTCCCCTCCCAAAAAAAAAAAAAAAAAAAAAAAAAAAAACCCTTTCC
CCATCCCCTTCTCCCTCCTCAAAAAAAATTAATTTCTAACTCAGTCATTTGGTGACATTTTTCTGCCCACTGCAAGACTCTCTTATAATCTTGC
ACATAGTAAGATGATATACTGAACAATAAATTAAGCTTACAGTTGCTGTATTTTAGGTAAAGCCAGGAGACCCTAGAAACCCATAAATATTTGG
AAATAAAGAGCCAGGAGTTCAAATGAGAAACATCTGAATGTGCCCATCCCCCTCACACACGCAGCAGGAAAAATTAGTGACAAGAGAGGACATA
GTGGAGAGAAGAGTCGGGAACACTGAAGCCCCAATGGCTGTGGTTAGAGTGAGTGGAAGAAAATTGGACATTGGAGACGAGAGAGACAGGAAGA
GGCTATGCGTAGATCAGATGCTAGGCCTGTGGGGAAAACTCCAGGCTCACTTATGGTGGGAAGGAGAGAGCAAGCCAGCTCAGCAGCGGTCAGG
TGGGGTCTTGTTGTTGTTACTGTTCTTAGGGACAAACTTAGGTAATTGAGTAAGACCTGCCATGCCTCTTCCCCTGACTAGGCACACAGATGAT
TACAAACCTGCTTCGATGGTAAGGACCAGTGCCCAGCAAGCCAGAAATGACTACTGCCCACTTCGTCTTGCTATGACTACTGCAAGGCCCCCTG
TTGCCTGCTCTTTCTGCCACCAAAACCACTAACATCCCAGTAGCAGCCCAGGAACTGCCCCCTCCCTGGTCCCCTGAAGCATAGCTATGGCTCC
CTCTACATCTCTCAGATTCACTCTGACACTAAGGGGCTCTGAGTACTCTGCATTTGGTATTAAAATTCGGAATCTGCAAGTGTTTAGGTTTCAT
TAGTGTGTGGCAGATGCTTTCTCATTGTAGTTAATTAGCAGTTGTTAGACCCTTAAACAGCCATCCAGACATGACACTAGTACTGTCAACAAGA
ACAAGGGAAGACACCTCAGAGACAAGAGACCAAATAAGTTACCCATAGGAAACCAAGGCTAGTCAGCAGGAAGGAGAGCTGGACAGCTAACGCA
CTGCCCAGCTCTGCTCTCATCCAGTTGGTTCTCTCAGGAGTCTTCTGTGTACCAGACATGCCAGGCTCTGAGCTGGAGACACACACAGTTGTCC
CAAAGACATGTTCCAACCCTCAGAGACAGTGCTGCCAAGAGATACACTAATGATAACCTATGCATCTGTCACATTTCAAGCTATAATAACTCGG
AAAAACAGGCTGCTATGGCAGCATGGAGTTGGGGAACCTGTCCAAATGTTATTGATCAGGCAAAGTCTTTAAGCACGTAGCACTCAGGCCAAGA
GCAGAGGGGTGGGGAGGGTGGCACAGCAGGGAGTTTCATCTCCTTCTGAGTCTCTAAGCTCTCTAATTTGGCCCAAATGAAGCACTTACTCCAG
CCCCTAGCAGGACCCCTTATGTGCCCCATTGCTGGGTGACCTTAGACATACCCTTCTCAGTCTCAGGTTTTACCCCTGCACCCTGCATAGGCAT
ATGCAGAGAGACCAGGAGAAACCAACTCTCTGACATCCCACTCTCAGAACCACACTGTCAGGCCTGGGACTTTGTACCCTCTGGAGGGAGGAAG
CTAAGACAAGAGCAAATAGAAGTCGGAAACTAAGCACTGCTTACTTCTTTTCAGTAATAGCTTGTCATTGCTTCATTTTAACCTTTTGTCTTGA
AAAAGAAAACATTAATTTTATTTTTTTTTTAAAAATCTGAAAACCAATACAAACAAACAAAAACTACACCTTTAATCCTGCCAAGGCAGAGGCT
GGTGGATCTCTTGAGTTCAAGACCAGCCTCATGTATATTGTGACTTCCGGGCAGTAAGCGACACAGAGAAACCTTGTCTCAATAAACAAAAGAA
CAAACAAAAGTTGTGTTGAGGCTGGAGAGATGGCTCACTGTTCAGGCTCCAGAAGCCCTCAGTTCAGTCCCAGTACCCAAGTCAAGTGGTTTTA
CAACCTCCTATCACTCAGACTCCAAGTGAGTCCACATTCTCCTTCCGCCTCAGAGGGCACCAGCACACAAGTGCATAGACTTACACACAAATAA
AAGAAATGTTTTTAAGCTTGTGTTGAAGGTAGCTACTAAAAATATGGACAAAGCCATTCTTTAAATTTTATCCTTTTAAATACTTGTTGCTTGG
TGTGAAAAATACTTTGTCTTACAAAGCAATAATGCAAACTGACTGTATGTAAGTCCCCTCCCACCCATCACATGCATACTCACTAATGCCCTCC
TGTCAGAAACCCTCAGATGATGCAGACTCTGTTCTTTATGTAAATTTTGCGTATCTTTTTTGTCATTGTTTTGTCTTTGTTTTGTGATTTTTCT
GTGTAACAGCCTGCCTGTCCTAGAACTTGCTCTGTAGACCACGCTGGTCTAGAACTCAGAGATCCACCTGCCTCTGCCTCCCAGGTGCTGAGAT
TAAAGACATGTGCCACCACTGCCTGGTTTAATATCTTTATTATACCTTAGAAAATGAGAAAATGAGAAAGCAGATTAGTATAGCTGGCTGATAC
ATTTACTTTTTAAAACCTGAAATCTTAAAAAATCTTTACTCAGAAAATCAGAGTGAAAGCCACAGGTTACCACATCTGTAATCCCAGGACTCGA
GCCACAGAGGCAGGAAGGACCAGGAGTTGGTGGGCACAGCATCTTCAGCTACATGAGTCTGTCTCAAAGAAGGGAGACCAAGATAACTGTCTGA
ATTGTGCGTAGTTTACTGCCAGAACCCCATCCACTGGCCATTGTTGCAGGAGTCTGAGGAGGACCCCAGGATTCTTTTCCTGCCATTTATGGTA
CAGGGTTTTTATGTATGCCAGAGAGAAATATCCCATTTTACTTTAGATATTGTCAGATTATAGAGCCATGGATTGCTCTAAGTGAGGATGTATT
TAATATCTGTCCATCCATTCATTTATTTATGAACAAGAGAATTCCAGCTGAGGTGCGGTCCTGGCTGTACACTCGACACTGAGGAAAGCCTGAC
CCCCTGACCCCAGTGGCTTGGAGTTGAAGCTGGACGGACAGAATACCAAGGACAGAGAGCTGTGTAAAGAATGCTTTTAACGAAATGTTAAAAC
AAAATGACTGCTCAGAGAATCCCAGATCATGCAGGCGGTCGCCAGCACTTAAGGAAAGAGAGAAACTCACTTTAAATGGAATGCATAAGAGTGG
AAGAGAGTGATAAGCACTTGCCTGCTCCAGTGTTAGGGTGAGATGCCACAGAGAGAAATCAGAGGATTTTCTGCAATTTTGATTTCATACTGGC
ATGAAAGAGATGGAAAAGAAAATTGCATTGCTGGAAAACGCCAGGGTATATTTGTTAACATTGCTATGGTGTACTGTATTTAAAGCAAAAATGT
AATTGCTGTAATTTATCACGTCTGGCTATTAAAACCTCCATAATTTAAAGATTTTAATCATTTGCCACTTTTGCACATAGTGACTTTTCGTAGA
TGATGCTGTGACAATTCTGATTTTTATCAGCCTTTATATGCTTTGTGGCAGCTCCTAAGCATCATTAGTGTGATTTCAGCATGCTATTGTTTGC
ATAACTAATACAAAATCCTTGTGTTTTTTTCCTTTGGGCTGTTCAAGCAGTGCCCAGAACTGCTTTATAGTTGCTGACCCTAACATGAAAACGC
AGGCTGCTAGGTCCATCTTCATCTGTCCACATCTTTCTAACTCTGAGACAAGCAGGGTGGGCTGTGCCTGTCCTTTAGATGTGGAAAGCGGATT
CTTCTAATAACCAGAGACTCTTGAAAGTGTCTCTGCCTTTGAAATGAGTGTGTGTGTGTGTGTAATGTGTGTTCAGGTGTGTGTGTGTCTATGT
GTGTGTTCATTTGAATAAGAATGGTCCCCCACAGGCTCATATATTTAAATGTTAGCCATCAGACTCATAAGCTGCCATCCAGGCATGCCCTGTC
AAGATGGACAGAATGGGTAGGAGGAAGTGAGAAGTCAGCCACGGGCAAAATCATTTCCCCATGGTATGTGTAAGAAATAGAAAATATGQCCCAT
TTCAGTATAATGTATGGGCTCTATCCATTCACTCCATCCTAGGGGAAGCTGGAACTTCTGTGATTTGTGGCACCTCATAAGACCAATTTGTGAC
TTTATATACCTCAGGACACAGGAGAATAGTATGATGACCTAATCCTGTGTGTATTTATTAAAATATAGCATTATTTGAGAGAGATATGGGAGTA
TTCATGTATACATACATATTATCTTAGACACTGTTCTGTTGCTATGAGAGACACCATGACCAAGGCAACTCTTATAAAGGAAAGCATTTAATTG
CGGATGACTTACGGTTCAGAGGTTTAGTTCATTATCATCATGGTGGGAAGCATGATAGTGTGCAGTCAGATATGGTGCTGGAGCGGTAGCTGAG
AGTTCTACATGCAGATCCACAGGCATCAGGAAGAATACCACTAGCCTGCCTTGGGCTTTGAAACCCCAGAGCCCACTGACACACTCCATCCATA
AGGGCCACACCTACCCCAGCAAGGCCACACCTCCTAATCTTTATAAGTAGTGTCACTGCCTGCTGACTAAGCATTCAAATGCAGGACCCTCTGG
GGGCCATTCTTATTCAAATCACACACACACACACACACATGCACAAATCAGGAATCATACTCCAGACCAAGCACATACCATTCAGTGTGATCAT
GGAATCGTGTTCAGAAAGCTAGCTAGCTCTTCTCTTTACTGACATACAAGAGTTTTGAAGTCCAACTCTTTGAAGAAAGATTTAAAGTTATTTG
CTTAAAGTCACTGAGCCAGGCCCAGGGCATAATTCATCGCCACACCCGCTATTCCTTCACCATCTTCTTCCTGTTCTCGTCCCCCAACCTGTCC
CCTGCAGATGACTTTCCAATACTTCGTCTTCCCTCTTCACCCCAGTGTAGGATCTCCGGCTCGTGAGTAACCGCGTCTCCATTCTGCAGGTGCG
AAGTTTCCCATTAAGTGGACAGCCCCCGAAGCGGCCCTGTATGGAAGGTTCACAATCAAGTCTGACGTATGGTCTTTTGGAATCTTACTCACAG
AGCTGGTCACCAAAGGAAGAGTGCCATACCCAGGTAAGGACACAGACCCGAGAAACGGTTTGTCTGTTGGGTTCTATTTTAACTTCATTTTACA
GGAGTCTTGCCCAGCTGCCTTTCTAAGCCCTGTGCTCAGCCTTCTGCCAAGGTATTTAGCCAGGGGAATGCAGAACTCAGAATTGCCGTGACCT
GTACACCAAATTCAATGTGCTAAGCAGCTGGTATAAATGTTCCAGTTCCCACAGGTCATGGAATTTGTCCTGTCTCAGTAGGTGCATGTGTCAT
GGCTCCCACAGCAGAAATGTCCCTGGCAGAGCCTGTGCCCAAATGGAGCTTGTAGACACGTCTGGGAAAGGGAAGGAGAAAGGGTCCTGTTGCC
TTTTGATCTCGTAGCACTGACCCACAGAGCAAGCCTGGCAGGAAGATGCCCGTCCTGTTCTCTCTGAGGCTCCAGCCCCAGATGGCCCCAGGCG
GAGGTGCAGGAAAGGCAGTTTAGGCTGACCAACCCATTCTCTGCCTGTGGCTGAATGGCCAAGATACACAAAATGGAGTGTATTATGAGGGTGC
GGGGGCCTCTGGGAGCCTGCAGTTGTTCTGGGCTGTCAGATGCTCCCTCTGTGAAGAAGCTGTGGGTTAAGGTTGCCCATGCTTAAATTCATTA
ACTTAGCCAGACGGAGACATAAAGGACACGCACCATTTCGCCTCCACACTTTACTGGTTTCAGGGCAGGGACGGTCTGCTTTGTGAAATGTTAA
GGACATATTTTTCAGTTACTGCTGAGCAAAGAAAGAGCATAGCTGGCTCACTCCCTTCATTCCCTGCCTCTTAAAAAGGGAGAAAAGAAAAGGC
CCACAGTGATGGCACCGATAGTAGAGATTAATGAGGAATGTAGGGCTCTTGCAAATCCTGGGCTTGTAATCATATCTCTGCAGTCAGAGGAGCC
AGCGAGAGCCGATTAATTTGTAATCCCTTTGTTCATTTTGTCTGATTGTTTCTGGTCATCATCAGAGAACAGAGAAGTGTTCATAAAGAGCCCC
TCTGTCTAGGCTAGTACAGCCTCAAGACCTCTAAGGGAGGAAGATTGGCACTGAGGCAGGATTCACAAGTGCCAGGCTCTGAAGCCCCAGCCCA
GCATTGGGAGGTGTGGGTGTGTGCGTCTTCTTTATTGCTGCTGCTGCTGCTGCTGTGGCTGCGGCTGCTGCAGCGGCTGCTGCTTCCGGCATGC
ACAAGTGGGCAGACACTCTGGGAATAGTCAACTGGCAGAGAAGAAAGGGCTGCCCAGGAGGGGGTGGGGCCCAGCAGGGGTGCTTTTGAATTGC
TTCTCAAGGATGAAAATATACCGTCTCCAGGTTTTAAAAGTGAGAAGGCACTTTTTAAAAATTATTATTGTCTATATTTTGAACTAATTTCTTC
CTGATCTCCCCAGGTTTATAGACACCCGGTGTGTTAAAAGCAATCACAGGAAAAGGTTAATGTTGTTTTGCATTTCCGTGAAGCTTTCTCTTAC
AGTAACACAAAGATGCTTTTTGTTCTTGGGTCACACACACACACACACACACACACACACACACAGAGGCCAAGACAAAGAGGTGGGTGTGGGG
GCTCAGAAGAGGAAGTATTTGGAGACCGGGGTTTTGTCAGGTTGGGTTTTAGCACAAAGAAAATAACCTAGGTTTACAAGTCTGAGTGGATGCC
TTCACTCAGCATCATTTTGTGTGGCCTGGCCTAGCATTTGTTCCCTTGGGAAGTAGCACAAATGAGCCATTCAGCTCTCAGGTGGCACTGATGA
GTTCCTGTCCCCGAGCAGGTACTATTCGAGCTGGAGGGAAAACCATGAGCCACTAAATAGTCCTGCCCTCACGGAGCGTTACTGCGGGAGAGTG
TTTGTGCCGTGCTTATGGAGGGGAGGTTACTTAGATGGGCAGACTGTGTGTGCTCCCCGACCTGAGTTCTCATTATATATATGGCAGAATCGGG
GACACAAGATAGCCCTTCATTGAGAGACCCATGAAGAGAGCAGAGGAGTTAAGTGAGAACTGGGTCCAGCAGAGTATGTGTATTTACTATGCCT
TCCATGACTGCAGCTGCCTGCAGGGGCCATGGGCAAGGGAGAAGGCAAGTAAATGAGACCATGGAGAGCAAGAGGCTCGCTCACGATGAACAAG
ATGTGTCGTCCTGCCTGTCACCCGGAAAAGCTGCAGAGACTGTAGGGAGGGTCTGAGACGATGGATGAGGTGACGTCCTTGATGGCCTTAGAAA
GGGGACCGAGCCAGTTGTGAGGGGTCTGTGTGCTGCCGTGAAGAGCCATTGGAGCCTGGCAGGTTGACTTGATGGCCCAGAGAGCTGCCTGCAG
CAGAGAGGCCATTCCAGACCTGTTCTCATAGCCACGGACACTGCTTAGTTTTATTTTGTAACTCAGCTACTCAGTTGGGCTTCTGAAAGTGACA
GAAACTCTAAGGGAGGAGCTCCTGCAGAGACATTATAGTTACAGGGTTGCTAGCCTAGCAGAGGGTAGCACTTAGCATTGTCCACATGTCACCA
TGGTTGTCGCTGTGCCCCAGTCGGCCAGCCTGTGCTGAGTGCTCACCTTGCTTCAGCCCTCCTGCACTGGATCTCCCATTCCCTGCTGGTGGGA
GTCCAGGAGAAAGTCCAGGTCTCTGTGTATCAGATGCTTCCATTATGCACTTTGACACACTAATTTCTCAGTTTCCTAGAACATATTTTCATGT
AATTCTCTTCTCAAGAATGGAAGGTGGTGATAAACTTGCCTGAGTTCTTTCATACCTGAGATTGGCCCTTTGTCCCCACGTCACATAGGAAAGT
GCTCTGGCTCATAAGTGACAGTGAAAGGAGATGGGGGCTGACTTACAAATGAGCTTGGCAAAGATCCCCAGACATTTCCACCCAGCATGTAAGT
CAGTGCAGTCAGCGTAGAAGGGAGCCTTGGAGAAGAACTGAGTGATGCTAGAGGCGTGGCCAGATGAAGGGCAACCTAGCACTGAGTAGGCGCT
GCTGGTGTCCTCAGGCTGTGCTCACTGAACTGACCAGAGACGGAGGAAGGAAAGAAGGACACACTGAAACTGAACAAGGCCTTGAAGCCGTAGA
GGAATATTGAAGTGTTGTCCTGAGGTTAAAGGGAGCTGGTCAGGTCCCTATCCACTTCCAAGATGGGTGACAGAGACGGACACCATTGCAACGC
ACAGCTCGCCTGCTGCAGCCCTGTGGGCTAAAAATGGGGTCCCGCATCCTGCCCAGGGCTCAGGCTGCTGGGTAAGGCAGGACATGGGAAGTCT
GACTTGGTGCACCCCATGCCACTGTCAGGCTTGAGAAGCTGCAGGACCCTGCTCTGGGGGCAGGGTAGCTCATTCTGAGGTCTCTGCGGACCTG
CCGGGGTTGGCTTGGGGTCCCCGGGCCTGCACAGAGGCTGAGGACAGCAGGTTCTAGCTCTTGGGTACCACAGAATGCCTCAGAAGAGCTGAGA
ACAGAGTAGGGGCCTTAGGGGCTGGCTCTGGAAAAGAGTGGGAAGAGAGCTCAGGCTGGTTCCCGAGGGGAGGAGAGTCTTTGGCTTGCTCAGT
GGTCAACTCAGGTTGGTTAAAAGGGTGGCTGAGAATGCAGAGAGGCCTTCTGTGTGAGATTAGGTTCTGTTCTTTAGAGCATTGTTCCCCACTG
CGGGTCCCCATGAGAAGAGACAGTCCATGGTCTTAAGGTGTTTAAAGAGAAGGAAGAGACTAGGGAAGTAGAGACCGGCCATGGCCACATTGGA
GGGAGGGGGGTGCGGAGAGGAGAGCAAGAGAGAAGCAACCCAGAAACAGGAGTAAGAGAGGGAGTAGGGGGCAAACATCCCTTTTGAAACCTGA
GCTTGCCAGGTAACGGTGGGGAGGAGCATATCTGGCTGTTGCCAAGTAACCGTGGGGGTGGAGTCCAGACAGAATACCAGGAGCTTGGGGCCCT
GTGTGACTGGTGGCCACAGAATTATGGAGCTGGGGCCTCATGTCAGGAGCCCAGTGTCTGGGGGCGTAGCAAACGCCTTCTGTCCCTTGCAGGA
ACACCTGCTGGGTCTCCAGGGTTTAAACCTAACTCAACCAGAAAACAGGCTGCCTTTCACAGTCCTAGACAGCCTATCAGCTGTGCCATCCTTG
CTGGAGTCCAGCCTGCTTTCTGTATGCAGCGCTTTAAGTGTTCTTTGCATCGAGATTAGAACTGTGTTTAGGCTAAGGGATTGTTGTATGTGCG
CAATGGTAATGCTGGCGCCAAAGCTGTTTCCTGAGCTTGCTGAGTAAAATCGTAGCTTTGCATTTTAGCATGACATTTCACCTCTAGAAGCTTT
CTCAAATTCTTTCCCCCTCCGCCTTCCCTGCCCCTGCCTGTATCAGGCTGTCACCACTGGGTGGTGACCTCCTAATGGAACTTCCTGCCCCCCC
CACCCCGTTCCTCTCCCTTCCTCCCCTCATCCTCCACCATTTTGTCATTGTCGCCAGCAGAAATGTTATCCTGGAAAACAGATGAATTATCTCA
CTCTGGTCCTGATTAAAGATGCCAAGGGCTCTTGCCCTTGCCCTCCAGATAACTCCAGAAGGCCGCACCCTGCACAAGTCCACAAATATTTATT
GAATGCCTGTTATGCATTGAGTGCTTTTCCAGACCCAAGGATTATAGCAGTGAAGTAACACGAGAGTTCCTGCCCTCAGGAACGTTTTTTTTTT
CTTGAAGAATATAGGCAATACAAACCAACAAACTACACTCACCCTGGCTAGTAGCATTGAGTGGAGCAAAAGAGGAATGTGGTTAGGAGATGGT
GACAGTGGAAGTCACTAAGAGGTGCTCGCTGACTATAACTCTGATAGGAGCTAGCCTTGGGCCAGGTGGGTGTCTAGGGGCACTGTGCCACCTG
CAGGGAGCCAAGGAGCCAGGGAGCCAGAGGCCTGGAGCGATGACTGGTGGCTTGTGGGGTTGTAAGCAAGCATGCTTTTTACTCTTTTTGTTGG
GGTTTAGTTTTGAGCTTGTATTCTCGTTACAATGTTTCTCCTTGGCCTTTCCTCCTTCTAAACCTTCCTAGTTACCCCTCCCCACACACTCTTT
CAAACTCATAGCCTCTTTTTTCATCAGTTGATATTGCATGCATAGATGTGTCTGTATACACATGCATATATATTTCTAAATACAGCCTGCTAAG
TCCATACAGGGTTGCTTGTGTCACCATTCCACACTAGATGACGAGTTGGTGTGCTTTTCCCTGGGGAGGGCCACCTCTCGCTCCAGCTTCACTC
AGTTGCCAATAGTTTTTGTGTAGGGTTCAGGTCTCATTGGGCTTTTTTTCCATTCAGTTGACATAACCATTGGTGTCCTCCTTCGTCAGGTCAT
GTTTAGGCAGTGATGCTGGTGAGACTTTACAGGCGTAGCTTCTGATGTTACTAGGAGACAAAACCTCACAGAATGCTGGTCCCCTGGCTCTTAG
AGTCTTTCTGCCTCCTCTTTGCAGTGTCCGGAGCCTTAGGTGTAGCTGTGTATTGTAGGTGTGAGTGTTCTCCTAACGTGTGTGCAGTGAGCAG
TGTGAGGGGAAGACTTGACTTCGGGTGTCTCTGCTGTCTCTCTCCCTTTACATCTTGGACACCCCAAGGAATTTGTCTATGACAGCATCTCACT
GGGTTAGCCGAGAAGTGGCTATGTAGACCAAGCTGACATCAACCTCACAGAGGTCTCTCTGCCTGCCTCTGTCTGGAACTCCCGGCAGCTCTGC
CTTCCTTGAGAGGAACGGGGCATCTTCCATGTTCTGATTCCCAGCAGTGTCTGCCCACAGTAGACTCTGTAGAAGCAAATAAAGGGAATGTGTG
AATAAAAGGGGAAACATTTGAACTTTTTTTTTAAAAAAAAAAAAAAGGAAGACTCATAGGTTTTTAAACATTTTTAAAGCTGCCCCATAGCCTC
CAGAGCCTTATAACTCTGCCCGGTTATCCTCACATATGGCCAACACGGGGTGAGTGTTCCCAGCCGCAAGGTAGCCTCTGTCCCCAGAGTTCTG
AGAAGTTGACCCCACGTTCAAATTTCATATTCCAGTAACCCGATAGGCAGAATGTCCTGGCCATGCCAAGCCGTGGCCCTCGACTGAACACCTG
CTTGTGTGCTGGCGGGATCTGGCAGAACAGGGCTCTTCTCTTAAGCAAATCTGTTTGTCTGTGCATTGGAAGCCTCCTCAAATCGTTCTTTCAG
CAAGTGTCAGCTGCTTAACTTCCTTGAGCAGAACTAGTCACTTGGTAATGACCTGGAGGAGTAAGGTGAGCAAGCAAAACTGCTCGCACCTCCT
GTAAAAGCCCAAGCGCCGCTTTTTGCCCCCTTCCTTCCCCCCCCNNNNNNNNNNNNNNNNNNNNGCATGATCACATGCCATGAAGACAGGTATT
CTTGTGTTCAAGCTGCTGACAGGGGCAGACTCCCTCAAGGAGGCAAGTGTGGACTGTCCAGGTAGAGGGGCCATGAGAAACAGTCTGAAAACAG
CTGACCTTAGCAGTGCATGATGGGTCCTCTGCCTCCGCAGCCCATGGGCCCCAGGGCCAAAGCACTGCAGTACACTGTGTTCCTTCAGAGAGCT
CGGAATGCTGTCTCCTCCCCTGCAAGGTTTCTCTTGGCTGACGTGGCTGATAAGGGGAAGAAAGGATAGGAGGCTGTCCGTGAAGCTGGAGGGC
ATAAGAAGGGGCAGTCTCCCAGTACAGCCTGAGCGCAGCGCTCTGCCGCTGTCTGGTCTTAGCTGTGGTCAGGCCCCTGCCTCCCACACTGGCA
CCTTCACCCTCTTTTTTCACTCCTCTCTTCAGAGGACAGAGGAGTTCTTTGTAGCACTGTATTATCGAAAGCTCAGGCTTGGAATCCCAGCACT
TGGGAGACAGGAGCAGGATTATTACTGTTAGTTTCAGGTCAGCCTGGGCTACGTAGCAAGCCCTTGTCTCCAAAAATAAAGAAAAGAAAAACAA
GAAAGGAAAAGCCCAGAAAGTATAATGCTGCATTATCCACTTCAAATCACCTTTGTGTGCCGCAGCTCTCCCAACCAGCATTTCTGATTTTATT
AATACAGCGGTTTTAATAAAAACTGCTGCCCCAGGTGACTGGTGAGGGGGCTAACAGTAAACAAAACTAGTAATTATTGTCTTCTCAGGGTCAG
TCCTTCATCTCCATCTGTACCCTCTGGTCAGTATCCCTCTGCTCCACATTTGTACTCTGATCCATGGAGTGGCTCTCCAAACCTCCTGGTTGTT
CCTGTAAGATAAGGGAAAACACTGGGTAATCTGATAACACATGCAGCGGTGCACTGCAGGCTCGCTGAGCAAGCACCATTAACTCCACTGGCCT
CTCTCTCAGCACCCGTGTGGCAGCTCACAACAGCAAGCAGACAGATGGGTTTTTAAGAGAGCTTATACAGGGAAGCAAGGGGCCTGAGACAGAA
GGCCCCATCCATACACAATATAGTATCATTCTCTTTGAATGAAAAGCTCAGTTCGAAAAAGAAATTTGATATCAGATAGCCTGAGTATCATCTC
CATTGTTAAAACATTTATTTACACCATGCAGTTGTCCCGATTCCCACAGCCATGGTGAAGTCCACGCATGACTAGAAAATAAACCACAGACTAC
AGGTACGTCTGCTCTGAGGTCCCTTGAGAAGCCACACAAGTGGGAAGCAGTAAAGTATTAATACTTTTTTGGTGAGACCTTGTGCTAAGGTGAT
GGATAAGGTGTTCTGTTAACAAAAGGAGGCGCTGAGGCTGGAGCAAATCGGGTCCCCTGCCAAGAGGTACAGAGTCTGGAAGTGACATCTGTTC
GGCGTTCTGCCTGTTGAAGTGGCTTTTGTTCCTTTCCTCAAGAGCCAGAAAGAAAAGTGAGGAGGGTCTACTGAGTTCTTGGACAGCTCAAAGA
ATAATACTGATGCCTACAAGAAGAGGAGAGGCAAGGACCCAAAGGATAAGGGAAAGAAAACATGGGTACATATGACAGTCTGCGTGGCAACTGC
ACAATGTCTGAGTTTGAAGGAAGAGTTGGAGAGCTGACTTCTCGTGTTTGAGAAGGAAGCCAGCAAAATGACTCAGAAAGAATAATACAGAGAT
CATGTCAGGAAGTGAGTGCAGTCCACTTGGCCAGAGGCTCGCAGCTACAATGTTTTAGGCAGAAGGACTGGAGCTGTTACAGCCATGACCATGT
TTGAAAACATGAAAGAAGGAGTTGAAGGGACATCTCAGAGCAGCAGGTTGTGGAGTTCTGTGAACTCAGGCCCATCACCTGGGTCCTCGGAAAG
AGTGGAGACATGCTGTTCTCCTGAGCTCTGGAGCAGAGGAGAGCTGAGATCAAACTGGGTAACGGAAGGGTATTTTTTAAGAATATTATCATGC
ATGGTGTACTTAATTGAAGTACTGTTTATGAATGGCAAACTCACTGAGTCCTTGCTGAGCCAGAACATCTTGACCATTGCAAAGGTTCACTGCA
GAAGTGGCAGGTTTGCAGGGCCGCCCACCCACGTGTGTTCTGGCCTTTGGTTGGTTGGAAGACAGCTATTCTGTTGCACTCGTCACTCAGTCAG
TTGCAGTGTCTACTCTTTTGACAAAGCTGCCGTTGGTATGTGTCTGTTTCAGCCAGTGCGTAGAATTGTTCATATATTCCCTGGACTTTGGGAG
GTCTGGAGAGAGTACCTGGAAAAATAAAAGGAGAAATGGGGTGGGGGTTGGGTGCTGCAGGAAAGGAGGCAGCAAGCTAAGAATGCATATCAAA
GCTGACAGTGTGGCCTTGCAGTCTAGTCCAGTCCTCACTCTGCATATACTGAGTATACTGCATATATTCTGTCCCATGGACGGAGGCCTTCTCA
GCAAATGTGACATGCTGTTTTGGGTTGTCACATGTACTATGTCCAAGTCTTGGGTGGAGCACTGGAAAAATAGGCTTGACCCAAATGGCCAGTT
AAACTTTCCATTGGCAGAGACTGAATTAAACGTTTAAAGAAAAGAAGTATTTCCATTTGAGGAATTTGGATTTTTAATGAAATAAACCATTTAC
TGACAAAAAATTCACGGAATTCTGGGAAAGTAGTTAATTGAATCCTGTGGTGCTGTGCTGTGCACTCAGGGCCAGGTTAAAGCCTTGAGATGTA
TTTGGGGAGCGGTTGGAGCGGTTGGAGCTGCTCCCTAGAAGCCAGATTCTAGGGAGAGTCTACTGTCATACCTCTGAGCTTCAGGCCTCTATGG
ACCTTTCCTCCAGGGGCTCCTGGCAGGAGCCAGAACAAGGGAGATTCCTGCAGACCCAAGCTGTGTAGTTCCAGAAGCACCATGAAACTAAGAG
GTTTATCTACTTAATTGTGCAGCAAGGAATTGCCTGAGAGAATACCAGCCCCTATGTATGGCTGTGTTCTTGTCTTGGCCCTTTAGGCTTCCTG
GAATAGAGACAGGTGGCTGTCTTGGAGCCCCAGCTTCTCAGCTAACCTCCCATGGGAGCAAATGCACATTGGAATCAGACATTGTTTTAAAGGC
AGTGCACTGATGTGTTACATAACACAGAAGGGGAGTCTGGGATGGAACATAGGAAGGAAGTGCCATCAGGGCTTCACAACACAGAGGGTTTATG
GCTGCTTCATGTTTGTAGAAATTTTTAATCCCAGTCCAATGTTTCTAGCCACTTTTGACCATTTAGTTAGCAGGTAAAAGATGCACACAACCTC
TATATTTAGAGTAAGCCTCAATCAGCACTAGAGCTGGGCAGATACCTACCATCTATGCTATTAAGCCTACTTTCCTATCGATAACCCTGAGCTA
TTACTTACTGTTTCATTTGAGCTGCACTTAATTCCAATTGGCCAACCCTCAGGGCCACAGTTTCAGGCCTCCTAACCCATGGCACTTCTCCTTC
CTCCTCCACCTTCTCCTCTTCCTCATTCCCCCTTGTCCCCCACCCCAGGCCCAGGAAACCTGAACCCCACCTATGTCTCTTCTGTCCAGCTATT
GGCTGTTGGCATTTTTATTTACCAATCAAAAATAACTTGGGGGCAGAGTCACAGGTCTACATGAAAACTCCAGGTCTTGCCCCACACACACACA
CACACACAATATTACAATAGACAAGAAGACCAAACCCCAACAGGTTCAGCTCTGTTATTCTGCCGAGTGCCATTTCCTGAGCTTACTGGGGTTT
GAACTGTTCCTGAGGAAGTGCTGGCCTCCGTTCCTTCCCTTTCTGTCTGGAGTATCTCAGAAGAGAGCTGCAATCCGTGTACCTTCCCTTGCCT
TCAGCTCTCCATCCTAGATCCTGGCTCCTCAGTATCTGGGGGAGGAATTGAGCTCGTTGGCTCCAGGGAACACCCAACAGAAAAAGGTACTCCC
ACACTGGCCTCAGCATTCTCCCAAGGCAAGGGGCTTGCCTGGCTAGAGTGAAATTATTTGGTCATATTAAAGGTCTACTTAAACACAGTCAAAG
GAATGCTAAATTTTGTCACCACCAGAAATTATAAGTACAGAGAATAAAAGAGCATCCTACAAGGACTTAGACAAACAGATGACTGAAATAGTTG
TCATCCAGGAAGACCTAGCTAGGATAGGTCCAGCTTCAAAGATGAGAGCAACCCAGAAGCCTGGATGCTCCCTCCAGAGGGAAACAGGAATGTG
ACCTCTGGCCAAGTGACAGGTTCTGACCCAGTGGTCACCTCCCCCAGAATTAAAGAAGGCTGTGTTGAATATTATGTCGATTCCTTACTAGTGA
CAACCCTGAGACTCATGCTGTAAATTACAGCAGATTCTTGCTGCTGTAAACTAACTGTCAAGAGTTAGGGAAGATTTTAGATGTCAGGCTCACA
AGGTCTTGGCAGAGAGAGGGAGAAGGAAGGGAGGAAAGGGAGGGAGGGTAGGCGGGTGGGCGGACAAGGGAGAGAGTACCTGGAAGCAGCTGAG
AAGGGAGCACACTGAGTGATGCCTGCTTCAGCTTCCCATGATGCCTTCTGCTCTGCAGCCTGAGAATGTCAGCATTAGGCAGCTCCTGCAGAGG
GGCAGGGAGGTCTCAGATGATAGATGGCCGGATGCTGTCATTCATAAGCAAAGGCCCCAGAGACACCCTGCCTCCCTGTCACAGTTCCCAAACC
TCAGAGCACTGTGAGGGTCACCAGGCCAGATGGTGCCAAGCCCTGCTGGCATGAAGGCAAACCTCAGACGCCATGCCTAAGCATGAGGAAAACT
GTGACATTCACTGTAGCTCTGCACATAGAAGACCTGTTGCTTTTATTCTACAGGGTGTTTGTGTGCTTTAGTCATACAAACATTTCTTTTTTAA
TTTCTTTAACTTTACATTGGTGTTCTGCCTCCATGTCTGTCTCTGTGGGGGTGTCAGGTCCTCTGGAACTGGAACTACAGGCAGTTGAACTAAC
TGCCATGTGGGTGCTGGGATTTGAACCTGGGTCCTTTGGAAGACTGAGTCATCTTTCCATCTCCCGAAGATTCCTTTTTAATTATGTTTCTTGT
TAATTGTGTGAGGATATGTGCACGTATGTACCAGTATCTCCTGAAACCAGAGGTGTCAGTTGCTCTGGAACTGGAATGACAGGCCGCCTAGTAT
GAATGCATGCTGAGAATTGAACTTGGGTACTTTGCAACTCTAAGCAACATTCTAGCAACATTAAGCCCCTTCCCCTAGTCCTGTGTTTTACCTT
TCCTAGTGAGTATAATTTACACATAACAAAGGTTAATTTAAAAGTATACTCCCATGAGTTTTGACTAGTGAAATTTGATTGTTCACACGGACTA
ACTACACTGGGGTCATGGACTGCAGCCTGAGTTATTTCTCATGGCAGAGCATGGCTCCCAACCTGGCTTTGTTTTAAACTGTTTCAGTGACAGA
AATAAGACAGAGCTTGCATGTAGCCCACACAAACAAAATATTTGCTGTTGGCCTTTCACAGGAAAAACATGGGCATTCAACCATGGCTTCTGTC
ACCTCTCAGCTCCCTCTGCCCTTTCACCCGTGGCCCCCACCCCAACCCCAACCCCCATCCGTGCGCAGCAACCTCTGATCTGGTTTCTATCATG
CAGTCTCTGGCCTTTCTAGGGTCTCATCCAGGTGAAATCGTACAGTGCGCTGCCTCTTGTGTCTGGCTTCTTTCACGTGTAGTGCTTTGAGAGT
CACTGTCTGGGCTTTTGCATCTATGAGTTCTCCATTCCTTTGTTCCACTCCTTCGTGCTGTGATAGCCTATTACAGGCATGGTTCCCCTCCTGA
GAGGGAGGACGGCCTCCTTATGCTTTAACCTTAGCCACGTGCCCATTCTCACTGGCCCCTGTCTTCGCAGGCATGAACAACCGGGAGGTGCTGG
AGCAGGTGGAGAGAGGCTATAGGATGCCCTGCCCACAGGACTGCCCGATCTCCCTGCACGAGCTCATGATCCACTGCTGGAAAAAGGATCCGGA
AGAGCGCCCGACCTTCGAGTACTTGCAGGGCTTCCTGGAGGACTACTTTACGGCCACAGAGCCCCAGTATCAGCCCGGTGAAAACCTGTGAGAG
CCTGCGCTTCAGACGCCTCTTCCCGAGGCCTCCCTACCCCTCCCCATTAGCTTCCAATTCTGTAGCCAGCTGCCCCAGAGCAGCGAGAACCGTC
CAGGATCAGATTGCATGTGACTCTGAAGCTGAACTTCCACGGCCCTCATTAATGACACTTGTCCCCCAGTCCGAACCTCCTCTGTGAACCATCT
GAGACAGAAGCGTGTTATTTCTCAGACTTGGAAATGCATTGTATCGATGTTATGTCAAAGGCCAAACCTCTGTTCAGTGTAAATAGCTGCTCCT
GTGCCAACAATCCCAGTGCTTTCCTTTTTTAAAAAGAAAAAGCAAATCCTATGTGATTTTAACTCTGTCTTCACCTGATTCAACTAAAAAAAAA
AAAAGTATTATTTTCCAAAAGTGGCCTCTTTGTCTAAAACAATAAAATTTTTTTTCATGTTTTAACAAAAAATGATCAGGACAGGTGTTTGGGT
TTTTTTTCCCTTTTTTATACATATGATATATATGTTAACATATGTTCCTGTACATACACCATGTGGGTGCTACCATGGAGACTGGCCAGCGTAG
GCCACATAGCTACAGGACCGGAGTGGGGATTACTGCAGAACCCTGCCACCAAAGCACTGGTGTCAGCCTGCAAGCCGGTGGCCTCATTTTTTGA
CTTCTACGAAGCATGACGTCCTCTCCATTTGGACTGCACTTTTTTGGTTCCTAATCATACCTATAGATTGTTCATGTGACTTTTTTCAGGTCCC
AGGGCCTAGTCACGAGTTTTAGTGCCAGTTTTTGCTCCAGCGAACTGTGATTCGTTCTTGAAACTTAGGAGTGAGCATTTTAAGCAAAAAGCAG
CCAGCCAGTTCTACCACAAGAGCTGCAAGACGGAGACCACACAAACTTCCTGTATAAATATGAATGCTGAAGGGTTCAGGTGTTTTCCTTTTAT
TTAATAAATCTTGTAACCACATTTAAATGGTCTAAACCCATAGCATTGTAGTCATGGCAACCTACTAAACTTTCTCATGCAACTAAAAATTATG
GGAAGGCAAGGGTGGGGGGTTGTACACGTCCCATTGTAAAATAAGTGTTTTACTGTCCTGTACTGCTAATGAGTGACTCTCCGTGTCAGGAGTG
CTCCAGTGAATAACTATGCACTACTTTACATTTCATGGGGGTGCACAAAAAAAAAAAAGTATTACATTTTTAGTTGCTGTTTGTACCAACCTCG
AATTACGTATGTTTAACAACAAATCAATTCCTATTTCTATTGAGTTTTTTAATACTGACTAGCAATTCTGAAGTCTTAATTCCTTTTTTTTGTT
AATGATTTACTTGTGAGTTTACATTTTTAAATTGTTTAACTTTCCTAATTTAGTAATTAAAAAGAGAGCATTTTACATTTGAAAAAAAAATTTT
TGTTTATTTGTGTACACCACAGAAGGTACCCTAGGCGTTCTGTGACATACCTGGCCCGTCCCAGATGGTGAGGAAGCACCTGGTAAATCGGGCC
ACTGTGCAGCGATTTCCAAACGACAGAAACATGCAGAGCATCTGGAGACCCTCCCCACCAAAAGGCAAAGCAGTTATCTGGCTAACTCAGAGTG
GCTAAAGCAAAACTGGGCGTACTTGAGCGCCATAGTGTATCCCGCACTGTCTCTCGCTGGAATTCACCGTGTCCTATTTGTACAGGAGACAGTA
GTCTTTCTTGGTCTCAAGTCATAAATACATTTCCAAGTATCCTACTTCTAAAATGAAGTTGCTAAGCATGTGACCATGTGGTATGGGGGAGAGG
GGGAGGGGGGACTGGGTAGCCATCCAGGCCTGGATCTACCATGGTTCCCTGCAGAACTCCGAACCTCCCCTGCAGGCCAGTGTGTCTGGAGACA
AAGTACAGACAAGATGCTCTCCCAGCCTCTGGCAGCCCTGCCTTCTTACAACTCTAAGTCCACCGTGTCATATGGAGCCATATAAGGAGGAAAA
TCTTTCATTGTTGTGGAAATAATTACCTTTAGAAAAGATTATAGCGTTTTGGGGAGGAAGATGAAACATTTTTTAAATCAAAATTGTTACTATT
TTGGATATGAATACCATTGATAGAGTTTTGAAAACTTACGAGGTGCAGTTTTTATCACATAGGATTAGTCATTAGCAGGTTTTAGATTTTTGTT
AAGATAATTTTATTTTCAATCCTCACTTTGCATTAGACAATTTGCAAATATTCCCTGGAGGGTTTTTGTTTTGACTCATTGAAAGTCTTTGAGG
GAAAAAAAAATGCACTTTTAATTTCATACAGCTTTTTAAATTTCATCAAGTGAGTTCTCTGTGGGTTCCTGTATATAAAAGAAAACTAAGGACA
TGCATCACTGAGAGAGCACGCTTACGAGGTCAGAGTCCTGGAGCACTCTAGAATAGAAGAACTACAGTCAGGAAGAAACCAAAGAAATGTACAG
GTTTTTCCAAGAGTCCAGTGTGCTTTCTAGGGAGGCTCAACTCCCATCTGGAAGTGTCTGCTTCGTAAGTGATAGACACTGGGATCGGGAAACT
GAAGTTGCTCACCAGAAAGTTCTCTTATGACAAAGGAAAAATAGCACTTACTGTGAAGCAGGTGAAAATGTTTGGGGAGAAAACCTCTTTGTTG
TTTCTCTTTAAAAAAAAAATAATAACATTTTTATGTGTATTGGTGTAAGGGCGTCGGATCCCCTGGAACTGGAGTTACAGACAGTTGTGAGCTG
TCATGATGGGAACTGAACCTGGGTCCTCTGGAAGAGCAGCTAGTGTTCTTGACCACTGAGCCATCTCTCCAGCCCCTGTTGCTTTCCTTTAGTA
ACCCAGAATGAGGACTAAGGACTTACTCTCTGAGAGCCAACTGGGGTTACCTAGTTTGCACTTGGATACCTTTTTAATAAGCTATTCTCTTAAA
AGCTCACATAGGAAGCTCACTGGAGAAGGCCATTTTCATAACACAAGAGTAAACATTGGTTCACTCCACTGTCGTTGCACCACACACTCAGGCT
GCTGCGTTGAGGGACCTGATAGGATCCCAGCAGATGCGGGGTGCCACATGTCAGCAGCGACTCAGTGGACTTTAAGGGCCCTGCCCCAGTGACC
AGGTGTTTCCATGGGGAAAGCCAAGGGTAACCATGGTCCCTCGAAACCTGCCGTGTGACTCAAGATCTCCAGGCACCTGGCTGTGTTTACTCAC
CCGGAGACGACAGGAATCCTGTGAGTAAAGTCTTGGCAGCAGAGGATTCTCTAGTACCAGCAGAGCTATGAAATCTATCTATGATTTCTCACGG
GGCTATAACAGCGCCCGCCGTATTAATTGGGATGGTTCTTTCCTGGGAAGGTAAGATGCTCAGCGGTACACCCAGGAGAAAAGCGCCATAGCTT
TGCTGCAGAAAAGATGCCATGTCTATTGTTGGCCCTGACGCAGAACAAGGCGGCCCACAGGGTGGGCGTTTCCTAATGGTGTATTTGAGTAAAA
TGGTATTTGGTCAGTTTGTTCTGGAAGATATTTTAATACATTCAGATGGCTTTTGTTGGCTTTCTTGCCACTGTGTGTTACACTGACCTTCTTT
CTGTTAGGCTAGTGTCAGAAATATACTTTCTGGAAGGGAGATGAGACGGAGGGAAGGAAGGAGATTAGCTTTGATCCTGAAAGAAGGGGAAAAA
CAGCCTGTGGCATCATTATCTAAGTTTAATTTTTTTTTTAATTCTCAAATGTAGATTCCAACTCTACAGAATGTAATAAAAATGCCTTTATTAT
CTTCTAAATATCAAATGAACCTTCACAACTGTGTTTATATCAATATTCCATGAAGTTTTATGACAAACATCCAAACTATTCCTCATTTTTCCCG
ATGTATGAAATATACAGAATCCTATGGGAAGGACATGGAGACCTCTTTATATATCTTTATTTTAAAATAAAAGCGGGAGAATATAGCTAAGTGG
TAAGCCACTTACTTTGCATGCCTTTGTCTCTTGTCTCTCTCTGACTCCCTCTCTCTCTCTCTCTCTCTCTCTCTCTCTCTCTCTCTCTCTCTCT
CTCTCTCTCTCTCACCCTCTCACCCTCTCACCCCACCCCTCTCAAACTCTAGGCATTGAAATATAAAGCGGTTTGTGATTTTGTAGGAACCACC
CTGTAAGAATGCAGTCCTAGACAGCAGCCAGAACAAGCAGACTTCCTCTCTGGAGTCCTCAGAACTCATGTCACTGTGTGCTTCAAACCACCCG
TGTGTTCAGTAACACTTCTCTTCTTTCAGTGGGTTTTCTGTGGCAGACTGAGCACTCCTGGAGACCAGAAACTGCCCAGCATTAACGATTCTCC
TCTCCCTCACCTTGAACGGAGATGAGCTTCTGGCAGATGTGGGGCCAATGCATGAGTGGATGGTTAGATGGATGGTGCCTACCACCCAGTGGGG
TGGCGCTTTGCCTTCAGATGACACGCTTATCTCAGAGCCTGCTGGGTGTGGGGGAGCTGCGGCATGCGGCCCCAGCACTAGCCTGGCCTGCTAC
AACTCGAAGTCATCAGTCTTCAGGATAACTGTGAAAGCTTTCTCTCCAGCCTGAACCAGGCACAACCAGTGACCCCACTCCTTCCCCCAGGTCT
CGGCACAACACCCTCGGTAGAAAGTCAGACTTCTTGGACCGTGCTAGGTGACTGCCCAGAGTAAAAGACCAAGTGAGGTATAGTTCACAATGCA
TGCAAGTGAGGTGCCATTTGAATTTGATAATTTTATGACAACTGGATATTCCAGTTCCTAAGATAACCATCTTTGTGTTCATATAAAAATGTAT
CTCAACTGGGCGTGATGGTACACGCCTTTAATTCCAGCACTTGGGAGGCAGAGGCAGGCGGATTTCTGAGTTCGAGTCCGGCCTGGTCTACAGA
GTGAGTTCCAGGACAGCCAAGGCTATACAGAGAAACCCTGTCTTGAAAAATTAAAAAAAAAAAAAAAAAAGTATCTCCATGCCCTCCAGATGTT
CAGTGTCCACGGCCTGTGGCTTGCAATGCCCCTATTGCAGCTCTCATGACCCAGGTACTTCTGGCCTACGTGAGAAGCTCTGTATGAAGTGGTA
ACTTATTGTAGTGTGAAGACTAAACGTGAAAAAGGTATTGTTGCCCCCTACGCATGGGGCTTTCCTGACTTCCCCACTCAAACAAGAACAGCCT
CATGGTTGAGTGGGAGGGAAGCCAAGGGAGAGCTTTATGACAGAAAGGAAAAGATAAGCCAGCTGGAGCCTCTGGAAGCAGAGGTCAGATCAGC
TTTCCTGGCCAGTCTGTTGCAAATCAGAGCAGGAGGGATGGAAACCTTAAATAAAGGAGAAGACTGAGCTGGGAAGAGAGGCGCCCTGGTCATC
ACTCTCTGGAAGAGTGGGAGGAAGGCAGACATGACAGGAGGTACACATGAGGACTAGTTATTCCTTGCTTTAAACTTCTGATGAACGTTCACTC
CGAGCTAGACCACGTGTACCAGAAACTCAAAGGTGTATGTTAATATTCACACCCCAGTGAAGACAGAGACCCAGAGCTGTCTGTAGATGGGAAG
CCCAGGGCTGAGTCGAAGGGAGGCACCCGTGCAGCATGGGGTCTGCAGGGCAGGCAGCACACCGTGGAGTGGGTTAGGTGTCACATAATAGGGA
CAGAACTTCTTGCTTTCAAGGTGACACCTGATTCTCATGCATTGAACACAGAAGCTGGTCAGTAAATGGATCTGAACAAAGAAAAAGCAAAGCT
ACATGCATCACCTCCCCACACACATCTGTCCTTCTAGATGGAGGTGTGGTAAATGGCTGCAGTCAAAGACGGGTAAGGTGCTTCGATGTGTGGG
TGGGGGAGGTGTAGCGAGTGCATATGGAACCTTCCTTACCTGCCAGCCTGTCAGAGATGCGGTGTCACTGTGCGCCTGTCTCTGCCTCCTGCCT
TGTGCAAAGAATGTTGGGATTACAGACATATGCCTCTGCCTCCAAATTTACATGGGCACAAGAGACCCAAATTCAGTTCCTCACAAAGGCAAAG
CAAGCACTTTTTTACCCAGTGAGTCATCTCTCCAGTCCCAAGAATAAGGGACCAGACAGGGTGTTGATTCCCAGGGTCCCTGCTGGAGTATGAC
TAGGAATATAGAATCCAAGAATTGCTGGCAAAGAACTTGTACTAACTCGGTCGCTCTCCCTGGGACATTTGACAGAGCTCTCCTGTTTGTCCGT
TCTTCGCTTGTTGGCCGCTTCATTTGCTCTGTCATACAATTTGCAAATTTATAAAAGTTACTGCTCAGAGTCACAAGGTGCTCTGGTTGTTTTG
TTCTGGGTATTTTTCCTTTAGTTAATTATCTAGTGCTTGCTCTGTAATAATCAATGGGTAGCTAATTGGAAGCAAGAGAGAATGAAATGAGTAT
TATTTGAATAGTGTTTTTAATGAAAAGCACACATGAAATACTATAGGGAAACAGCACAAGAGAATTTTTTTTCTTTTTTTTTTTTTTTTTTTTT
TGCATGTGTTTACAAACAGATTCACATAAGCTTGTCACAAACCAAATCCAAGTGTATGGATCATTTTGTTTCATTTTCCAGATCCCCCCCCCCC
CAAGGGGGTGAGTGGGCATTGGAGGATACCAGGCGGAGGCCAGGCCTCAGGAGATGGAGAACTAGAGCCCCAGCCGTCCTAAATGAAGCTGAAG
GGCTGTCAGTTCCCAAAGAGTGCATGAGGAAATGGTTAGCACGAGACAGCACTTGCATAGAGAATCAGAGATGAGTAACAATGGTTCCTGGTAA
ATTAGCCATATTACATCATGCACACAGTAACGGAGGGACGATTAGGAAAAATGAATACTAATTTGCCCAAATCCTTTACATAAAATGCATTGAC
TCTTTTTTTAATTTGATAGACTTTTTGATTTTTTAAAAACATGGGCAGCTCCAGGAAGCTCCCAGCAGAGCATCATTGTGAGAGGAGGAGGAGG
AGGGGGAGGAGGAGGAGGAGGAGGAAGATAAACCATCGAAGGCCTTCATAAGCCATTTCACCCCCCTCCTCTCAACCTGCGAATATCAGAAAAT
ATTTGTTTGTACATTCCTGAAGGTCTCTAAGTGAAAATGGAAGGCATTAATCATTGTCTCTGCTATCACTAAACCATGACCTCACCATGTTGGA
GGGCAGGAAAGTGATGCTTCCTTCTATCTGTATGTCTGCGCTGACTCAGACGGTCTCTGGGTTTTGAGTAGTAGGGGGAGGATTGGAATGGAGC
AGAGCTGTACTTAAAACGGCAAAGGCAAGAACAAAAGCAGCATGCGTGCAGCCAAAGGGGACCTTGGAAGTTACACAGATGAAACAAAACGCTA
TGCTTGCCTGAGAGTCTGGTTTCCAAAGCAAACGCAAAATGGAGAGCCTTGTTCCAACCATAAAGGTCTCACCATCTATAGTTCCTTTGCAATA
AAAGGGGCCCTGTAATCTACTGTGACAGCCAGAAGCTGCACTGGTGCTCTGAATTATGATATTTGCTCACGACTAATACAGATTTTTCCAAAGC
AGCGTAGCATTTGTAGCTATAGCATCCTCTGCCCTTTGGCAGAACACAGCCATGTCCGCCAGCTTTCCCTGCTTCCGTTCCTTCCTGTCCCAGC
CCCCACGCCTTTCCACCTCTTCCCATTACAATCAGACCATTCTTGCTTCCTCCTTTGTTTGCTTCGGCACACCTCCCTTCATAGGATCCCTGTT
AATTTATTAAAATCAGACTTTTTTTTCTTTCTTTTTTAAGATGTATTCTCACTATGCATCCCAGGCTGACCTCAAACTCAAGACCTGCCTGCCT
CAGCCTCCCTCCCAACTGCTAGGATCTGGGGCGTGTATCCCTTAGGTCTCTACCCTTCAAGGCAAACCTGTGGCTGCTGGACTGCATCCTGAGC
AGGCATCCTGTAGGAGAGTTGCCCAGTCTCCTCTGGAAACTTACTGGTCTCAAAGTTGAACTTTGTAACCCTCAAATCTCAACACTTCTCTCCT
GGGAGCCCATTTTAGGTAGGCAGTTCCCTGACCCCAGTTCTCCTCCCTGCCTCGCCCCCAGCCCCCAGCCCCCAGCCCTCCCCCCGACCTATGA
TTTATCAGACTTTATACCTGAGTCTCCAGCGACTTGCTATCAATCAGCTTTATGCAAATAGAAAAGGCGACCCTACCTCAACAAGCTTTGCTGC
CATCTGCCAGGAAATTGCTACAATAAATATGCAAAAGGATAATGATTCAAATGTGAATGCCACCCTTTCAAATCCTGCAGTACAAAAAAACGGC
ACAAACTGGGGTGTCAATCCTGCAGTGTGAAATCACCGGAGCTGTCCTGAAACCCTGCATAAAATCATGGCCCCATGAAGTCAGCCTTGCCTGT
TCGTAGCAATTTTAAAATCTAAGCCTTTCAACCGTGATCAGAAACTGTGGAGAGGCGGTCCCCAGGTAGCAGCCTGTAACAATGACAGACTTCT
TTCTAGAACTCAGCCAGAGCGTGGACTCTAGTGTAGGGCACACAAAAGCATCCAAGTTAAGGAGTGGGCACACTTGACCATGAGGGTGCTAGGG
CAGAGACCATGCTCAACCAGGGTGGGGCAGGGGGTGCACCACACCCCTCTGCCTTCTCACTAATCACTCCAGCATCACCTCCACTTTCTGGTAA
TCTACAACACAAATCAACCCCAAAACCTCATGGCAGAAATAGATGCACATGGGTGCATTTTGTACAGAACTCTGCAGTTCAGAGGGACTTCTAG
CTACGACTGGCCTCTGCTCCTCAGGGCCGCAGCTGGGACTGCATGGAGGAGGCTGGGCATCTTTCACAATAGCTCCTAGCTGCTGAGCAAGAAC
CCAGCTGGGACTGTGGGTGAGGACCTGAGCTCCCAGGTGGCCCGTGGGCTGGCTCACACCGTGGGGATCAACCGTGTTGAGACATCCCAGCTGA
CTCATGGCAAAGCAGAGACAAGCTGCTCCCACCAAGCCCCGCCCAAACCAAGTTTCTAAACAAAAGAAATGACTGAGGACTCTAGCTATGAGGT
TTGGGGTTGTTTTATAGCAGCTCATCACCAGAATGTGTAGGCTATCACAGCTCCCTCGTATGCCTCCCCTCATGCTGTGTCTAGGAAAATGAAA
ACCGAATCTCCCAGGCTGCTATACTGTTTGCACCACAACCGTGAATTAGACTATACCGGTCAGATGTAGTTGTGGAAAGTTTGTAAGGCCAAAG
GGAGGTATATGCCAGGCCCCTGAAGCTTGTGCTTGTGTATGCCCTAGGAAGCAAGAACATGTAGCCACAGCTCTGTAGCAGCAGCTCCAACATT
GGTTTTCTGTCTCTCACCCTCCTGGTTCATGCAGGAGACCACAGTTCCGCTGTCTGAGTGGTTCCCTTGGTGAACCTCTTCCAGCCCTTCCAGT
AAGTCCATAAATTGTTACATTACATCTCTCTTCACTTAAAAGCCATAGGGTGGGGCATGCATCCTAGGCTGACCTGAGTCAGCATCCCCAGGTG
AGCCCCACTGTTCCATCTCCTATCTCTCTGTCCTTGCTTGCTTGCTCGTTTTCTCTCACTGGGCACTCTTGTTTTCTTTCCATGCTGAGGATGG
AACCGAAGCCTCCAATACAATACACTTATCAAGCACTCTCAGCCCGCTGGTCTTTCTTTTAGTATTTATCTATACTTGTGCCGGTGGGTTATTT
CTCTGCCCGTTGAAATGAGCCAGTTCAAGCCAGATTAGATTATATATAGGCAGGTTTATTTGGGTGGGGAGGAAGGGTGGNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNCTTCACCCCCACCTCCACACACAATCCTTCTTTTTTTTTTTTTTTTTTTTTTTTTTTTTGCTCCTCCAAAAAAAAATGTCTGGAT
TGTATAGGGAGTAGCCTCTGGGGGAAGGGCAGCCCAGCCCCTGGGCTGGAAAGTTCAGGGTTGGGGGCAGGGTATGCCAGGTAGGAACTGAGAG
ATGCTGGGAGAACCTGCAGGCCAGGTCTGCTTTGATGTGTAAAACATGCACCTAAGTCCCTAAACCCAACAGTGCCAACCCAAGTTTCTCATGG
CTGAGAAAAAAGTCCATTAGCTAATGGGAGACAAGGAGTGAGGACTAGAAAAGGACACCCTCCGGGGCCTGTTGGGTTGCTTTCTTTTGTATGT
AAAGTTTTCTAGAAACACAGCCATCCCCATTTGATTCTGTCCTGTCCAGAGCTGGCTTCACTCAACACTGACAATGTTGGGTGACTGGTTGACC
CAAAACTCTGAAAATACTTAGAATTGTCTCTCTGTGGATTCTGTAGGCACTGTGGGTCAAGTACCGCCGCGTTCGGGCTAAGCACTTCCTGAAC
ATCCCCGCTGTCATGGGGACTTCCATCTGTCATCACTATTAGTCTTTTTCTGCTGCTTTGTTTTGTTTCTGTGCAAACAAAACAAAGACTATTC
TGGAACTCACTCTGTAGACCAGCCAGGCTGTGAACTCAGAGATCTGCCTGCCTCTGCCTCCCTTACTAGTCATGGGAGTAACAAGTAATAACTT
ACTTGTCAGTTATTACTGACAAGGAAACCAATTTAGAGAGATCTGGGCCCAGTTGATTGAGCACATGGCGATGCTGTTAGCAGGGATACAAGAG
CCAAAGGAACAGAAAGGACTCATGGACTGGTGACTGGTGAGGCACCTGCAGCCCCATGTGCTGGGCTGTGCCCTCTCATGTCTTGCTACATGGC
TAGGAAGGGTTTCAAATCAATACAGCATTGGGATTCAAAGCACAGTTCTGTCACCAGCTAGCTGTGTGACTCTGGGCAAGCTATTTGACCTCTG
AGCCTCTTGCTTAGCTTCAGGGTCCTCAGTTAGTAAAGGGGACTTTTGAGGAAAAAAAAAAAGTCCTATGTGTAGGATATGAAAGCAGAAATGG
ATAGTGTGTGGAGGTGGGGTGAGGATGNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNAGGTCAGGACAGGGCAGGGTGGGAGTGGGGTGGGATGGGGACTGGGTGAGGTGAGATAGGGGTGAGG
TAGGAATGAGCTGGGGCCGGGGTGGCATGGGGATGAGGTAGGGTGGGGTAAGAGTGAGGATAGGAGTGAGGTGGGGTGGGATGGAGTAGGGTGG
GGATGGGATAGGGGTGGGGTGGGAGATCAGCA
MOUSE SEQUENCE—mRNA
CCTGGGCCCCGCCGCGGACGCGCGGAGCCGCCTGGGCCGCGCCGGAGGAGGGCGGGGAGAGGACCATGTGAATGTGCTCCGGAGCTGAGCGCCA
AGCCAAGCAGTGTTTGAAAGGAAGCAGGATGCTGATCTAATCGTGGCAAAAAGTCAGTCCGACCGCTGGTTTCGAAGACATGTGGTGTATATAA
AGTTTGTGATAGTTGGTGGAAATTTGGGAGCTTGGATAATGGGCTGTGTGCAATGTAAGGATAAAGAAGCAGCGAAACTGACAGAGGAGAGGGA
CGGCAGCCTGAACCAGAGCTCTGGGTACCGCTATGGCACAGACCCCACCCCTCAGCACTACCCCAGCTTCGGCGTGACCTCCATCCCGAACTAC
AACAACTTCCACGCAGCTGGGGGCCAGGGACTCACCGTCTTTGGGGGTGTGAACTCCTCCTCTCACACTGGGACCCTACGCACGAGAGGAGGGA
CAGGAGTGACACTGTTTGTGGCGCTTTATGACTATGAAGCACGGACGGAAGATGACCTGAGTTTTCACAAAGGAGAAAAATTTCAAATATTGAA
CAGCTCGGAAGGAGATTGGTGGGAAGCCCGCTCCTTGACAACCGGGGAAACTGGTTACATTCCCAGCAATTACGTGGCTCCAGTTGACTCCATC
CAGGCAGAAGAGTGGTACTTTGGAAAACTTGGCCGCAAAGATGCTGAGAGACAGCTCCTGTCCTTTGGAAACCCAAGAGGTACCTTTCTTATCC
GCGAGAGCCAAACCACCAAAGGTGCCTACTCACTTTCCATCCGTGATTGGGATGATATGAAAGGGGACCACGTCAAACATTATAAAATCCGCAA
GCTTGACAATGGTGGATACTATATCACAACGCGGGCCCAGTTTGAAACACTTCAGCAACTGGTACAGCATTACTCAGAGAAAGCTGATGGTTTG
TGTTTTAACTTAACTGTGGTTTCATCAAGTTGTACCCCACAAACTTCTGGATTGGCTAAAGATGCTTGGGAAGTTGCACGTGACTCGTTGTTTC
TGGAGAAGAAGCTGGGGCAGGGGTGTTTCGCTGAAGTGTGGCTTGGTACCTGGAATGGAAATACAAAAGTAGCCATAAAGACCCTTAAGCCAGG
CACCATGTCTCCGGAGTCCTTCCTGGAGGAGGCGCAGATCATGAAGAAGCTGAAGCATGACAAGCTGGTGCAGCTCTACGCGGTCGTGTCTGAG
GAGCCCATTTACATCGTCACGGAGTACATGAGCAAAGGAAGTTTGCTTGACTTCTTAAAAGATGGTGAAGGAAGAGCTCTGAAGTTGCCAAACC
TTGTGGACATGGCGGCACAGGTTGCTGCAGGAATGGCTTACATCGAGCGCATGAATTATATCCACAGAGATCTGCGATCAGCAAACATTCTAGT
GGGGAATGGACTAATTTGCAAGATTGCTGACTTTGGATTGGCTCGGTTGATTGAAGACAATGAATACACAGCAAGACAAGGTGCGAAGTTTCCC
ATTAAGTGGACAGCCCCCGAAGCGGCCCTGTATGGAAGGTTCACAATCAAGTCTGACGTATGGTCTTTTGGAATCTTACTCACAGAGCTGGTCA
CCAAAGGAAGAGTGCCATACCCAGGCATGAACAACCGGGAGGTGCTGGAGCAGGTGGAGAGAGGCTATACGATGCCCTGCCCACAGGACTGCCC
GATCTCCCTGCACGAGCTCATGATCCACTGCTGGAAAAAGGATCCGGAAGAGCGCCCGACCTTCGAGTACTTGCAGGGCTTCCTGGAGGACTAC
TTTACGGCCACAGAGCCCCAGTATCAGCCCGGTGAAAACCTGTGAGAGCCTGCGCTTCAGACGCCTCTTCCCGAGGCCTCCCTACCCCTCCCCA
TTAGCTTCCAATTCTGTAGCCAGCTGCCCCAGAGCAGGAGAACCGTCCAGGATCAGATTGCATGTGACTCTTGAAGCTGAACTTCCACGGCCCT
CATTAATGACACTTGTCCCCCAGTCCGAACCTCCTCTGTGAACCATCTGAGACAGAAGCGTGTTATTTCTCAGACTTGGAAATGCATTGTATCG
ATGTTATGTCAAAGGCCAAACCTCTGTTCAGTGTAAATAGCTGCTCCTGTGCCAACAATCCCAGTGCTTTCCTTTTTTAAAAAAGAAAAAGCAA
ATCCTATGTGATTTTAACTCTGATTTCACCTGATTCAACTAAAAAAAAAAAAGTATTATTTTCCAAAAGTGGCCTCTTTGTCTAAAACAATAAA
ATTTTTTTTCATGTTTTAACAAAAAAATGATCAGGACAGGTGTTTGGGTTTTTTTTCCCTTTTTTATACATATGATATATATGTTAACATATGT
TCCTGTACATACACCATGTGGGTGCTACCATGGAGACTGGCCAGCGTAGGCCACATAGCTACAGGACCGGAGTGGGGATTACTGCAGCGTGATC
ATGCAAGCTCACCCCCTTCCAGCAAAACACTGGTGTCAGCCTGCAAGCCGGTGGCTCATTTTTTGACTTCTACGAAGCATGACGTCCTCTCCAT
TTGGACTTTTTAATAACCTAATCATACCTATAGATTGTTCATGTGACTTTTTTCAGGTCCAGGGCCTAGTCACGAGTTTTAGTGCCAGTTTTTA
GTCCAGCTCAACTGTGATTCGTCTTGAAACTTAGGAGTGAGCATTTTAGCAAAAAGCAGCCAGCCAGTTCTACCACAAGAGCTGCAAGACGGAG
ACCACACTAACTTCCTGTATAAATATGAATGCTGAAGGGTTCAGGTGTTTTCCTTTTATTTAATAAATCTTGTAACCACATTTAAATGGTCTAA
ACCCATAGCATTGGTCATGGGCAACCTAAACTTTTCTCATGCAACTAAAATTATGGGAAGGCTAAGGGTGGGGGGTTGGTACACGTCCCATTGT
AAAATAAGTGTTTTACTGTCCTGTACTGCTAATGACTGACTCTCCGTGTCAGGAGTGCTCCAGTGAATAACTATGCACTACTTTACATTTCATG
GGGGTGCACAAAAAAAAAAAGGTATTACAGTTTTTAGTTGCTGTTTGTACCAACCTCGAATTACGTATGTTTAACAACAAATCAATTCCTATTC
TATTGAGTTTTAATACTGACTAGCAATTCTGAAGTCTTAATTCCTTTTTTTTGTTAATGATTTACTTGTGAGTTTACATTTTTAAATTGTTTAA
CTTTCCTAATTTAGTAATTAAAAGAGAGCATTTTACATTTGAAAAAAAAAAAAAAAAAAAA
MOUSE SEQUENCE—CODING
ATGGGCTGTGTGCAATGTAAGGATAAAGAAGCAGCGAAACTGACAGAGGAGAGGGACGGCAGCCTGAACCAGAGCTCTGGGTACCGCTATGGCA
CAGACCCCACCCCTCAGCACTACCCCAGCTTCGGCGTGACCTCCATCCCGAACTACAACAACTTCCACGCAGCTGGGGGCCAGGGACTCACCGT
CTTTGGGGGTGTGAACTCCTCCTCTCACACTGGGACCCTACGCACGAGAGGAGGGACAGGAGTGACACTGTTTGTGGCGCTTTATGACTATGAA
GCACGGACGGAAGATGACCTGAGTTTTCACAAAGGAGAAAAATTTCAAATATTGAACAGCTCGGAAGGAGATTGGTGGGAAGCCCGCTCCTTGA
CAACCGGGGAAACTGGTTACATTCCCAGCAATTACGTGGCTCCAGTTGACTCCATCCAGGCAGAAGAGTGGTACTTTGGAAAACTTGGCCGCAA
AGATGCTGAGAGACAGCTCCTGTCCTTTGGAAACCCAAGAGGTACCTTTCTTATCCGCGAGAGCCAAACCACCAAAGGTGCCTACTCACTTTCC
ATCCGTGATTGGGATGATATGAAAGGGGACCACGTCAAACATTATAAAATCCGCAAGCTTGACAATGGTGGATACTATATCACAACGCGGGCCC
AGTTTGAAACACTTCAGCAACTGGTACAGCATTACTCAGAGAAAGCTGATGGTTTGTGTTTTAACTTAACTGTGGTTTCATCAAGTTGTACCCC
ACAAACTTCTGGATTGGCTAAAGATGCTTGGGAAGTTGCACGTGACTCGTTGTTTCTGGAGAAGAAGCTGGGGCAGGGGTGTTTCGCTGAAGTG
TGGCTTGGTACCTGGAATGGAAATACAAAAGTAGCCATAAAGACCCTTAAGCCAGGCACCATGTCTCCGGAGTCCTTCCTGGAGGAGGCGCAGA
TCATGAAGAAGCTGAAGCATGACAAGCTGGTGCAGCTCTACGCGGTCGTGTCTGAGGAGCCCATTTACATCGTCACGGAGTACATGAGCAAAGG
AAGTTTGCTTGACTTCTTAAAAGATGGTGAAGGAAGAGCTCTGAAGTTGCCAAACCTTGTGGACATGGCGGCACAGGTTGCTGCAGGAATGGCT
TACATCGAGCGCATGAATTATATCCACAGAGATCTGCGATCAGCAAACATTCTAGTGGGGAATGGACTAATTTGCAAGATTGCTGACTTTGGAT
TGGCTCGGTTGATTGAAGACAATGAATACACAGCAAGACAAGGTGCGAAGTTTCCCATTAAGTGGACAGCCCCCGAAGCGGCCCTGTATGGAAG
GTTCACAATCAAGTCTGACGTATGGTCTTTTGGAATCTTACTCACAGAGCTGGTCACCAAAGGAAGAGTGCCATACCCAGGCATGAACAACCGG
GAGGTGCTGGAGCAGGTGGAGAGAGGCTATAGGATGCCCTGCCCACAGGACTGCCCGATCTCCCTGCACGAGCTCATGATCCACTGCTGGAAAA
AGGATCCGGAAGAGCGCCCGACCTTCGAGTACTTGCAGGGCTTCCTGGAGGACTACTTTACGGCCACAGAGCCCCAGTATCAGCCCGGTGAAAA
CCTGTGA
HUMAN SEQUENCE—GENOMIC
TCTCTCCCTCTTAATTTTTCAGTTGACAAAGTATACTTCATCAATGTGTAGTTGTTTCAACTTACATACAAATTCAATCAGTAACTGATAATAT
TAAAGGGAACTTTCTTATAGAAAAATAAAGGTGATTTATTCTCATTATAAAAAAAGTACATTCTTGGTATAGACAAATCTGAGAAAAAAGACAC
AGAGAGCAAAGAAAGAACAGTTGCCCATAGTGTTATCATCTGCACACCACCATAATCTCAGCATGTTTCCTTCCAAAGAGTTTTCTAGGCATTT
GTTATATAGTTGCAATCATGTGATCCTTCCTAATATAACATGAACATCTTTCCATATTGCTGATAATTTCAGTAAATGTAAGTTTTAATTTCTG
CTTAACTTGTCTTTATGGGCATACCTTACAATTTACTTTAGCCATTGCCCTCATGCTAAACAACTAACAAGTTTTCTAATTTTTCAAATGATAA
ATAATACTTCATAGAACCTCTTCATGTCATTTTTTTAAAAGGCGTGTCTTTAGCACACATTTACAGGTTAAAGGACATGCCACCCCCGACACCC
TTTTTTTTTTTTTTTGAGGCAGTCTCACTCTGTCACCCAGGCTGGAGTGCAGTGGCGCCATCTCAGCTCACTGCAAACCTCTGCTTTCCTGGTT
CAAGCGATTCTCATGCCTCAGCCACTCGAGTAGCTAGGTTTACAGGTGCCTGCCACCACACCCATCTAATTTTTGTATTTTTAGTAGAGTTGGG
GTTTCACTATGTTGGCTAGGCTGGTCTTGAACTCCTGGCCTCAAGTGATCCACCCACTTCAGCCTCCCAAAGTGCTGGAATTACAGGTGTGAAC
CACCACACCTGGCCGAGTATGCCCATTTTTAAGAATTTTTTATATTGCCAAATTGGTTTACCAAATGGTTTTACACTGATTTCTGGTCTTCTAT
CAGCAATGTAAGAGAGTCATATTCATTTCAAGGAAACTTCATCACTATTGAGTATACTTATTTTTTTAATGTGTGAATTTAGAAGTGATACATA
GTATCTCACTAATCTAAATTGTATTTCTCTGATTACTACCAAAGGCAATATTCTCCCATATGTTTCACAAATTGTTTTTCCTTTTTTTGTTGCC
AATATATGTTTCTGTTTCCTAAACTTAGAGTTTCTCTTATCTGTATAAATTTCTTATATATTGAAATATTAATCTAAGAGAGTTGAAAACATAT
CTCCACAGAAAAACTTGAGCATGAATGTTTGTTGCAGTATTATTCATAAGAGCCAAAACGTAAAAACCCAAATGTCCATCAGTTGATGAATAGA
TAAACAACATCTGGTATATCCATACAGTAGAATATCTTTTAGCCATAAAAAGGAATGAAGTACTAATACATGTTACATGAATGAACCTGGAGAA
CATTATACCAAGCAAAGGAAGCCAATCACAAAAGACCACATATGATGTGACTCTGTTGATATGAAATGTCAAGAATAGGCAAATCCATAGAGAC
AGAAATTAGATTCGTGGTTGCCAGGGGCTGGGGAGAGAAGGGAGTGGGTAGCGATTGCTTAATGGGCATGGGTTTCTTTCTGGAGCAATGAAAA
TGTTCTGGAATTAGATAATGGAAATGGTCTCCTTTGTAAATGTACTAAAAACCATTGGACTGTGTGCTTCAAGAGGGTGAATTTTATGGTATAT
GAATTATATCTCACTTTAAATGAAATTTTAAAAATATTAACTACAAGGGGCTTTTCTATGGTCCAAAAAGGCTTGCGGGCAGGGGGTGGAGCCT
GAAAAAACTTTACAACCTGTGCTCTAATTCTTTCTGAAACCTTTGGCATAGATGGGGTTAAAACATATCATCCTTGTTTTCATCACCATTTCAC
TCTTGTCTTAACCCTGAGATCCAGAGCAAAATGAATTTAAGAAGAAAATGTTCTTACTCTTTTCTTGGAGCCTTTCCATGGTTTGGTGTTAAAG
CCTCATTTGCCCATTTAGGTTTGGGACTGGTTCCAGCATTCAGTTGACTGCAGTCTTAAATCTCACCACTCGGGTCTGATGCTTCCATTTCTCT
TTTATTGAGCGCTTTTGAGTCTTCGGGCTCGAGAAATGGAAGGCTATGCAGAAAGGAAAGAACATGTTTGTTGCAGGGATGTGAGCAGCTGCCT
CTTAGCCTGGGGGCCATTTGAACAAGAAAAGCCAGAGATGGGGACCCACAAAAATATTCTAGGTGCCAGCAGTATGGCTTATTCTTAAAGTAAC
ATGACATTTTTAAGAGTGATTTATATTACTTTGGTTCCCTTGCTGTGCGGTATGAGAGAAGGTTGATCCTCTTCCCCCTCCAGAGAACCTTTGC
AAAGTCATGCGTAAATGATCGAATGGGTTCAACTCCACTTATTTCTGAAGCAAAGCTTGGGTTTGATTTTTAAGTAGGCTCTGTTCTCCCATGA
TTGCATGTGGCTAGTGTCTGCATTGTGTATATTCCCACTTTTACATCTATCTTCTCACCTCAGAGACTGTAATATTTTAATACTTGCATTTGGC
GAGGGGCTCTCCCTCACAGGCTTCACCAAGAGTAGAAAGCTGTTATTTTGTGCTTGGTGGAGGCATTATATGTGTATCTCAGTTTATGCAGTAA
ATATGTTCCAGAAAGTTTTGCCTGTAAAGCAAATCATATTTTAAGTGCAATAGGAAAGCTGATTTTTATACCAAGGAAACCAGTGCCAACTCCT
TCATGGTACTTATGAATACCCTTGCCATGACTAGGAAAGATGAGGAAATTAAGGGCTGCTTCAGTGTCATCGGCCCAAATAGTCATTAATATGC
CAAACCGCACGAAGGAAGATGGTGACTCACTGCACCTAGTATGGTCCAGCATCTCTACAGAATCCACTGTCTTGGGGGAATGGAATGTGCATTG
GGGATGGAGGTGAGACCTAGTCCCTGAAATCAGTGCCTCCGTTAGTAATGTGTACTAGTCCATTCTCACACTGCTGTGAAGAAATACCTGAGAC
CGGGTAATTTATAAAGGAAAGAGGTTTAATTGACTTGCAGTTCCGCATTGCTGGGAAGGCCTCAGGAAGCTTACAATTATGGCAGAAGGCAAAG
GAGAAGCAGGCACCTTCTTCACAGGGCGGCAGGACGGAGTAAGTGCAAGCAGTGGAAATGCCAGATGCTTATAAAACCAACCATCAGATCTCAT
GAGAACTCACTCACTATCACGAGAACAGCATGAGGGAACTGCCCCCATAATTCGATTACCTCCACCTGGTCCTGCCCTTGACACATGGAGATTA
TAGGGATTACAATTTGAGGTGAGATTTGGCTGGGGACACAACCAAACCATATCATAATGTGTGAGATATTGAGCTCTGTGGAACCAATTCTTCC
CTATAGGATTTTTCCCTCAACACCTCCAGCCCTCACACCCACACCCTTGCATGGACTGGCCAGGATTCCATGGTCAGGAAATAGCTCTAGTATA
GAGACTCTGTGTGTTGGCCATGGAATCAGGCTATCTGGTTTCAAATCCCATCTCTATGACTGACATAGCTGTGGACAAATTCCCCAACCCCTCT
TTTCCTCCCTTGTAAAGTGTGGGGAAATAGTAATACCTGTGTTTTAGAGTTGTTACTAGATTTTAATGGGACAGCATTTTGCAGAGTCCAGCCT
GCACCTAGGCTACAGGGAACACTGATTGGATATTGGTCATGACTGCTGAAATTGGAGCCATCATCCCCATGGTGCCCATCCCACCAAAACATGT
ATTAGTTTTCTGCTGCTGCCTAACAAAGTACTACAAACTTAGTAGCTTAAAACAACATCCATCTGTTAGCTCACATTTCTGTCAGTCACAAATC
CAGGCACGGAGGGGATTCTCTGCTCAGCAAAGCCGAAATCAAATGTTAGCTGATTATGTTGTTGTTTGAAGGCTTGACTAGGGAGGAATCCATT
TCCAGGTTCATTCAGGTTCTTGACCGAGCTGTTCTCAGCTCTGACCACCCATGTGTCTGGCCACATAGCCCCTTCATCTTTAAAGGCAGCTGTG
GAGAATCTCCCCCACAATGAAGGCCTCTCTCACTTTTAATCTACTTTTCCAGGAACTGGCATACCGTTCACGGGTTCACCTGATTAGGTCAGGT
CCACCCAGGATGATCTCTCAAAGTCGGCTGTGCCAAATAACAACCCGATGATGGAGTGACTACCCCATCATTCATAAATCCTAGGGATTTGCAG
GGCACATACACCGGGAGTAGGAGTCCTGGGGGACAGCTCAGAATTCTCCCTACCACAATGTCATTTAGCAAGGCACATATTTTCACTCCCTCCA
GTTGGCAGCAAAGAGAGGACTTTCCCACATTGGCAGAGTCCTGCATGCTTTTTTTCATAGCTGGGCTGGGGCCATTGGCTGAACCCAGCATCAG
TGCTGAAAGTGTTTCATCCCTCAAGTATTAACATCTCCAGAGGGCCAACCCAGCAGTGACTTTTTCTCTGCTTCAGTACCTTGGGAAATGCTTG
TGATCCCAAGAGTGGCCAGCATAGCCCTGCTTTCTGTCCAGCCTCCCCAGTCCTCCCTGGCCCTTCTGTCCCGTCTGTCCCCAGAAGGCAGCAC
TGCCTGCACTCTCTCATGGGGTGTCAGGATCATCTGATGCAGTATGCTACAGAGGAGAAAAGCCCCATTGTAAAGGATTGAAAGGTGCTGGGAA
ATTAGGAAATTTGGGTGACAGTTTCTGTGGCCCAACCTCATTGCTGCTGATGTGGTACTCAACTGTCTCTGAATGTCCTTCTGCTCCCAGTAAT
GACAGGGGAAAACTAAAGAAGGCTTTCCTAGGCTTTCAATGGATTAACTCTCTCCACTGAGAATGTAGCAAAAGTCCCCAAGTAGATTCTTATG
GGTGTTTCTAATTTTGCGAGGATCAAGAGCCCTCTGCCATGAATCTTCTATCCTGAAGTCAAATAATTTCAGATGTGTCTCTGCCACATCTTAT
CCTTTTCCTAATGCTATTCCTGCTGACTGAAAAAGTGTGTTTGCTTGATAAGCTTCCAGTTTTTGCTTTTTTCCGAGAAAAGTGTGAAACCATG
CAGTTGATCTGCAGTTCAGCTTTAAATTCTTTTTTCAACTGCCCCCATCTCAGTTGCTTGCATTTGGTTTCTAGGTTCTTAGTTCAGTTCCCTT
CCTTGTTCTTATACTTCCATTATGATACCAGGAAACATGCCACCTCTAAGTACGGGCCAAATGTGGCTTTACCTGTCACTCTCCAAAGTCTGCT
GGAGGCTGTCAGAAGCAGGGATTATGCCTAAGGGCTGTCACTTCTTCACACCCACCTAGGTTGGCTTCTGAGACAAAAGAGCATACACCCCTGA
AATTAAAGTTATTTGTTGTCGGTTTCACTTCCTCTTCCTGCCATTCCTCCTCTGCCTGCAAGTGTATGTGTCTCAAGAGAAAGTAATTTGCTTG
TGCAGAAGACTGCCAGTGGCCTTTTTCCCATTTTGGTACCTAGCTCCTGCACACACCTGAAATCTTATCACACGTCCTGCAAGCTTATAACTTG
GGCTTACTGGCATTGCTTTATTGATTGGCACATGACGAAATATGAATGTGTCTGTGTTTAATGTGCATCTTGTAGACTGGACTGTGGTCTGTCT
ACATCAGGAGGAGGGAGTGTATGTTCAACCAGACTCATCTTGGATCGAAGCACCCTGTGCCTGTGGTAGTAAAGGATTGAATCTATCCTTTAGG
TCAGTTTCCGTCAATCACTACAGGGCTATGTGGAGAAGCATATCTCCCATAGCATGGCAGCAGAAAGGGCTGTGATCTGTTGATAATGTCTGCC
CTGCTTATGGGAGCAGGGTGCGGCGTTCCTTGAATCCTGTGTCCGGCAATTCTTCCCAGGTTGCTTAACACTATTGGCACATCTGTAAAGAACC
ACAGGACTAGGGAAATTTTAAATAGATCAAGTTTTATTTGCTTAGTAGAAAAAGGGCCCCTCCCTTTGCAAAAGGATTGGAGAATAGAGGCTTC
ACAGCTGTAATCCAGACACCTTGATCACAGGCACAGGATCCAATTGGTGAAAATGCCCTTCACCAGTTTTTCTAGGCATGATAACTTGCAGAAG
ACTATAGCCATGAGTTCATGGCGTGTGGTATTTTCCTGATCAGAGTAACTCCAGGGCTCCACATTCACGATGGCATCATTCAGTGTTTCCCGGT
GCCCCAGAGATCTATAGCAAGTGAATTAGTACATTCTATGTCACGATGCACTTACAGCATTGGACATTAAAGCCACAGCACATAACTCTGCATA
ATAAAGGATACTTTTATTTTAGCATGAATGATCAAAAGTTTGCCTTGTAGCAACGAGCCTGGGCACACTGAATACAGCTCATGTAGCGTCTTCT
GACTCTTCTGTCACGAAGCAGTTAGTCTGACTGCCCCACGGGGGAGAAAGAAGACCACAAGTTCCTACATAAATATTTTCATTGTGATTAACTT
TTAATGACTTGAAACTGCATTTACTGATTTATTTTCACTTCTTCCTATGTGTCATTGAAGAGAAAAGAGTAAGCAAGATCTGTATAGAAACATT
ACTTAACTCAGAATGTCAAATGCGTGAAGTGGTGATGGTGCTGTCTCTTCTATAAAAGAGGATTGTTTTCTCCAAAATGATTTTGATTATTTTT
AATTTATTTATTTAACTAATATTTATTGAGTGACTACTGTGTGCCAGGCACTGTTCTGGCCACGCAAGATGCAGCAGTTAAAAAAAAACAAAAA
TTCCTGCCTTCAAGGAGCTGATGATTTTTCTTCTCTATTACTTCATAAAATTATAGATAAGCTGCTAGTTGCTTATTATAGTATATTTCTTTTT
TAAAGTGGATTTTGAAAGATTATTTTTTCTTAGTTTGGTTAAATGTAGGATCCTGTTTTCTTCAGGGTTCTTATATGGGGCTTATTTTGTTTTG
CTGGAGTTGGAAGAGTGGTTTGCAATGAGTGTTCTCATTACTTCCAGGTTCTGTTTCTCTATCACAGTGCACAAGCTTCTAGAGAAAATCAGAG
CACTTCATGTCACTCTTTAAAACTGCCTGCTTCTACCTAACATGCCTGTTCGCCTGAAGAAAGCATGATGGGTTGCATGTTAATCACATCCATG
TAAGGGAAACAGCTGTTAAACTGCTTCAAAGATGTAGATATAAATTCTTTCCTTCGTGTAAATCAGGATTTCTTGACCTTGACACTATTAACAT
TTGGGGCCAGGTAACTCTTGTTTGTAGGAGGCTGTCCTGTGCATTGCAGGAAGTTTTAGCAGCATTCTTGGCCTCTACCCAGTAGGTATCAGGA
GTATCCCTCCCCTCTGAGTGTGACAACCCAAAATGTCTCCAGACATTGCCCAATATGTCTCTTGCAGGGGAGTGGTAAAGTTACCCCCAGCTGA
GAACCACTGCTGTAAGTGATATGAGTCTGTGATTCAGGTCTCACATTCTCTAATTACAGTAGATCAGTGAAATTCATGGAAAAAAAGAAAAAAC
ATCTGGGACAAGAAGTGTTTTCTGTCTGGTCTTTGCAGAAATGTGGAGCAACTTTCCACCACGAAAGAGTTAATATTATATGAGCAAGTTATCA
GTCTGATGAAGCAACATCAACAACAAATGATGAAATTCCTTTAGTACATCATGCCTTGGAAATAGCTTGCATTTCCTATTGGAAACTTGTTAAA
ATCATATCCATTTACCATAAGTTACAAAGCATGTTGGGCACGGTATACTGTAAGTTAATCTCAAATGATTTAGGAGTAAATGTGTTCCAGATTT
GGTGAGCTCAGCTCAAGCAGTAGGCGTCTCTTTTCCAAACATTTTCATTCTCAAGAACTGACATTCAATTCAATAAGTATTTATTGAAATGTGT
AATATGAGAGAGCTTTAGAACAGTCTGATACTGCTCCCATCACCCAGTCCAGCAGAAAAGCAGTGCTCAGAAGAGTGCCCAGGGAGCACTCTTC
TGATATGGCACCTCCTGCATTGGTTTGCAAGATAGTATAGAAAAGCATGCCTTCTCCTTTGCTGCCTCAAGCCCTTCCCATCCTGTGCCAGCTG
TGCCCTCCCCAGTGAATAAATGTTATTACACATGATGGCATGTGTTTCTTATGTGATTCTTTGGTCCCAAATGGGCTAGTTTGCGGTTTTGGCA
GGGACCTCCACTGGCCTGTTCTACCTGACCCCTGTTGGCTTGTTGGGGTATTGCAAGTCCAAAGGGGGACCATTTAACCAACTTGTATTTCTGA
ATCTTCTTCACTTCTCACACACATACCAGTAGATTTTAAAATGCAAGGTACACTGCTGATAAAAGTCACAGGAGGGGCCTGTCACGGTGGTTCA
CGCCTGTAATCCCAGTACTTTGGGAGGCCGAGGCGGGCGGATCACCTGAGGTCAGGAGTTCAAGACCAGCCTGACCAACATGGTGAAACCCCAT
CTCTACTAAAAATACAAAAAATTAGCTGGGTGTGGTGGCAGGCACCTGTAATCCCAGCTACTTGTGAGGCTGAGGCAGAGGTTGTAGTGAGCCA
AGATCGCTCCACTGCACTCCAGCCTCGGCAACAGAGTGAGACTCCATCTCAAAGTCACAGGAGGGATCGGCTTTAATACAGAGAGGGTAAAAGA
GAAACATAATAGGCTACAGAATCTTCACTCATCTGGTTGCTATTTATTGTTCCAGGCACTGGCAATGTAGCTGTGATGAAAACAGACAGGGTCC
CGCTTTCCTGGAACTTGCAGTCCATTGGGCAATGAGACAAATAAACAGGCCCTGTTTTAATATGGCGCCAAGGGTGCCAGAAGGAGGGAGATGA
GGATGCCATGGGAGCACTTCAGAAAGCACAACCCAGCCTGGAAGGGCAGGGCAGTCGGAGAACGCCTTGCAGAGGAAGCGGTGCCTTATTAACT
AGGACATGGATACGTCACATATCAAAGAGGAAGGGGAAAAAGTGTTCAAGGTTATGTTCCAAGGCCCGAAGCGGGTGGGGGAATGGAGTCTTTG
AGGATCTGAGAAGTTGGGCGTGTTTGAATAGGAGAGCCGAGACAGGAAGAGTGGGAAGTAAACAAGGAGGAGGAAATGAGGATGCTTCTCAGGA
TGCTGACTGGAGCAACTGGGACACTGGTGGTCCCATTTACTTAGATGAGGAGCCTGGGAGAAGAGGGTTTGAGGAGGGTGGGGTGGCAACAACT
CGAGTTTTTAATCCGCCACTAACTCTGTAACATGGGAATGATTTTTTTTTAATGTTTTTGAAGGATGTCAAAAGCTAATGGAAATTTGAAAAGT
TAATTAGCAGCTTTAAGTGATTTCATTACTGTAGACTATCATGACATCCTGCAAATATACTCTGACCTCTTAGGTTCTGCCGATTTTATTTTTC
ATCCAGTAACTAGTTCTTAGTTTTGATACTTAGCATCATCTTAGTGCTTAAAATTGACCAGTTTTGTGCCTGAAGATGTGGTACCCATGAGTGA
AGCAGTCTAGTGACTTGATTGAAGGTTGTATTCTGGCTTATCCAACACTCCTGTGCCCTCTTCCTTAAATTTCCTGTGCCCTGATGGAAGTTTT
TATTCTAATGTTGCATAGACTCATTCTACAGAGCATGTATTATTTTTTTTAGATGGCTAAAGGCAGAATTGGTAAATATGATTGGACCGTCTGT
AGGCAGGTGTATGCTGTTACAGAATTTTACGAGATACAGGCCTTATTCGAGGAAAGTTGCTGCATGGTGAACATTGTGCTCCGTGATGATGCTG
TCATCCAGAACATATGGGGAACAAATGCGATTGTGGGGTCAAGGAACTGAACTTCCTCCTAACCATCTCCCCAGCCACTGCCTTTCCTCATGCC
CCCTAATAGGCCTGAAATAGCCAACAGTGAGCCTTGTCAATTACTCCTCGTTTACTGTAGAGCAACCCAGCAACTAGGGAGCATCCCAGCTTAC
GTGCCCCCTTTTTTGTCTTTGGCAGGAATTTAGATAATGGGCTGTGTGCAATGTAAGCATAAAGAAGCAACAAAACTGACGGAGGAGAGGGACG
GCAGCCTCAACCAGAGCTCTGGGTACCGCTATGGCACAGACCCCACCCCTCAGCACTACCCCAGCTTCGGTGTGACCTCCATCCCCAACTACAA
CAACTTCCACGCAGCCGGGGGCCAAGGACTCACCGTCTTTGGAGGTGTGAACTCTTCGTCTCATACGGGGACCTTGCGTACGAGAGGAGGAACA
GGTAAGCCCCCAAAGTCACAGAGTGCAGAGAGGGGGCAGCCACCCTTAAATCTTTTGGCAATTGAACATCTGGGTTCCCATCACTTCCCTTTGA
CTAGGGGTTTCACATGTAAGTTTCTAATCTGTCTCCTCTACTAGATGAGGGGTTGTAAGAAAAGCCAGAGTTTGATGTACAAACAAAATGCTTG
GCTACAGGGTTGCAAGGTGCCCCAAGTTGCTGCTTTCTGAGGATTCTATATGGGTTAGTGAGCTACAACATTCTACTGAGGCACTGTAGGTCAA
TGACTCTCAGCCTCACCTGGCTGGTTGGCACAGAAGAACTAGGGCAGCCTTTTCAAAACACGGATGCCTGAGCCATGTCCCAGTCTTTTGAATT
AGAGGCTCCAGGACTGGGTCTGGGTGTGTGTATTTTGAGTTCTCTAGATATTAATGTACTCCCTCTTCCCACTCACTTCTTCCTAATCCTCAGC
CTGCCTTTCTTTTTTTTTTTTTTTTGGAAGTTTCCTTGTCTTTCATACCACAAGGCTATATTCTGCTGCCATGTGTATTTGCTGACTTAGCTAA
TACTTTGTAAAATATCTGGCGTACGTTCTCAGACATATGTTTTCCGAAATATTTGTAAGTTATGTCATGCATCCCTAGTCATCCAAATTTTCTC
TGAGTATGTGAACCATGGTAACTAGATATTTCACATTATTTGTTCCAGAATTATTTTGTTTATTGCAAAAGTTTGAAAGAATGCAGCATCTCTC
AGTTCATCTGGCAGCAGATGTCTTATATAAAAGTTTGAAAGAATCCAGTATCTCTCAGCCCATCTGGCAGCAGGTACCTTTTCACCTAGGGAGG
AGGCACTACTAAGGCATTAGCAAGTTTTTTTTCCCTCTGCACCCAACACACATCACACATCAGTGTAGTTGTATCAAGGAGCCTGACCCTGAGC
CTTTGGACATTATCTACTTCTCTTCTGCTAGCATAGCACACTCTGCCCAGTAGAAGAAGTATTTATGTTCACCCTCATGCTGTGGTGACTCTAG
CTTTCCTTTTAACCCAGGCCTTGCATGGGTTGAATACGTGCTAAGTACCTTTACCCATCCTTCCCACCTTCCCAAAGGTGCCTGAAGAGACACA
GACAGGCAGTCTAAGGCCATCTGTCTTCTGATGATGCAGGAGAAAAAAAGAGGCAAACACAACCCCCATCCTTGTGCCTACCGTGAGAAGCATA
AGGGCTTTGCCAGCCCAGACTGTTAATTCTACTGACACAGCCATTGTCCTTGTTCATTCATTCACGAGTGCTGGATATGGACCCCTGGGTCTGC
AGGTATCATTCATTAAAGTCACTAGAGAGGGCCACTTGTGTTGAGCATGTGGGTCTCCACCGTAGTCAGGAGGAGTCTTTGTTTAAGAGGCTAC
CTGATCCCTTATAAAATTTGAAAGTTAAAACACAGTACTCCCAAATGTTGGCAGATTTTTCTGCCTTGAGTTATTTGGTTTTAATATATATGCA
TGTCCTTTCTCCCAATACTGCCTTCAGTCCTGTAAGGCCAAGAACAACGTCTGTGTTTCTCTGTAACCTCCTTTGATTCTCCCCTCAGCTCCTG
GAACATGGAACATAAAAGCCCTCAGTGAGAACTTGTCAGCTGATGCAAGGAGAGAGTGGGGTGCTGGTCTTCATGTGCCTTGCCTTCCACACAG
TGGGGCTGGCTTGGCTTCATTTTTAAATTGTTTTTAAAGAAGGGAATCTGGTCATGGAGTGTTAAATAAGAATAGGCATGACCTTACGGGCACC
CACATTTTTGTTTTAAGCCAATCTCTTTTCTTACCTTTCAAAGAAATCTAAGGAAAGAAGGAAAGTTGCCAAATGAGCCTAGCTTTGCATGGTC
AGACCTCTGAATGCTAACTCTCCTGAGGAAAGTCCCACTTGGGATGGGGCAGAAAGACAAGCCCCACTCTTCTCTGTGGTCCTTGAATAGTGCC
ATGACACATTTCTTCCCTTGGAACTACCTACACAGCAAATTGGCAGCTTGGTAAAAAAGGTGGACTCTGGCCGGGTGCAGTAGCTCATGCCTGT
AATCCCAGCACTTTGGAAGGCTGAGGTGGGTGGATCACTTGAGCGCAGGAGTTTGAGACCATCCTGGCCAACATGGCGAAACCCCGTCTCTACT
AAAAAATACAAAAAATTAGCCAGGCGTGGTGGTGGGCGCCTGTAATCCCAGCTACTCGGGAGGCTGAGGCAGGAGAAGTGCTTAAACCCGGGAG
GCGGAGGCTGCAGTGAGCCGAGATCACGCCATTGCACTCCAGCTTGGGCAACAAGAGTGAATCTCCATCTCAAAAGAAAAAAAAAAAAGTGGGC
TCCCTGTCATTTCTGTAGGTCATAGCAGGCACTCTGAAGTGCAGAGAGACCAGAATGAAAAGGCAGACTGCAGGGAACTACTATTAGTTATCAA
CTGCTGTATAACAAATTGCCACAAAACTTAGCAGCTTAAAACAAAACACATTTAATTTCTTTTTTTTTTCAATTTCAGATGTTTTTTATTCAGA
GGTTCTTAAAAGAAATAAAACAGAAAAGCTAACAGTCTGATCAAACATACAGCTCAAAAATGTCTTTTGACGTTTAACAACAAGTCCTAGGAAA
GAAAACTACAGAGTTATCTTGAATGGGGTAAATAAGTTACCACTGGCAAGTCTGTGGCACTAGTAAAACAAAAATAAAAAATTAAATCTCGGCC
AGGCGCAGTGGCTTACACCTGTAATCCCAGCACTTTGGGAGGCTGAGGCAGGCAGATCACGATGTCAGGAGTTCAAGACCAGCCTGCCCAACAT
GGTGAAACCCCGTCTCTACTAAAAATACAAAAAATTAGCTGGGCATGGTGGCAGGCACCTGTAATCTCAGCTACTCGGGAGGCTGAGGCAGGAG
AATCACTTGAACCCGGGAGGCGGAGGTTGCAGTGAGCTGAGATTATGCCACTGCACTCCAGCCTGGGCGACAAGAGTGAAACTCCATCTCAAAA
AAAAAAAAAAAAAATTAACTCTCTTGATCATATAGATATCTCTATATCTCTTTTTTTCAATCGGGTACAAAAGATCTTTCTTCATAATTTTTTT
TATAATTTAATGGCTATCTACTATATGATGTTTAACTAATTTTTTTTTTAATATACAGAGGTTATGTTTCCCAAATCTTACCCAGACGAGTATG
GCACTTAGTTAGACATTTCTGGCAACAGATTTCCCCTCCCCTCTAACCAAACTATCAAATTTCTGCCTTAACTAATGCAGAAGTATCAACTAGT
GGGAGACCCAGGTTTATAACTGTACTAGGTACACAGAGGGTGCTGCGTTCAGCCTGGGCTTTCACAAAGCAGTAATATTCCAGGGAGCATAGAA
CCAGTGATACCGTGACTTTTAAAAAATAAAACAAAAACAAATTTATCTTATGACTATGTAATTTTCACTAGAATCTACACTTCTCAGAGCAGCC
AGGACTTTGGAAACTATGAAATGTCACTGACATTTATCATCAAATACAAGCATAAAATGTAAGAACTAAAAAATACTCAAAAGAAATAACTGCT
TAGCCCTAGCTTCTGAGCTAGGAGGATTTGGGCTGTGGAAACAGGGAGGGCCAGCAACCTGTCCCAGCACAAAAGAAAATAGACTCTGAGGTAG
ATAACTGATCATACACAGCTGACCAGACAAGTACACAGTTTTGGAAGGCAACTTAATTTACCATTGCTTTTAAGACATATGCAATTGCCTTAAT
AAAAGAGCTTTAATAACAGTGTATTATCTTATATGTACTAAAGTAGAAATAACTGTGTATTTATGAATCAGGAATTTCATAGTTTCAAAACTGG
TTTTCTTTACACTTGAACACTTGTAGTGATGACATAAAGTGTGGCACCGCTTGGATCCAAATCTTCCTTCATGCTTTTTTGTTTATGTGTGTCC
ATACATCAATTAAAATTATAAAACCTAAATTTACAGTTTCTGTGGGTCAGGAATCTGCGAATGGTCAGTTCGGTCCTTCAGAATCTCTCACAGG
CTGTAATCAAGGTACGTTGGCTGGGCTATAGTCATCTCACAGTTTAGCTGAGAATGATCTGCTTCCAAACTCATGTAGTGCTGGCAGGATTCGG
TGCCTCGCTGGCTGTTGGCCAGAGGCCACCCTCAGTTCTTGGTCACATGGGCCTCACCAGCATGGCGCCTTGCTTCATCAAAGTACACAAGCTG
AAGTGGCAATAGAGAGAGTCTGCTAGAAAGACGGAAGTCACCATCTTTTGAAGCCTAGTCACTTAAGGGACTGCACATTCTGTTGAGTAGAAGC
AAGTCGTTAGATCCAGCCCACACTCAAGGGGAGCGGCTTGCACGAGACTGAATACTAGGAGGCAGGAGTCACTGGGGACTATGCTAGAATTATG
CCTGTTGGGTTGGGCACAGTAGGTCACACCTATAATCCCAGCACTTTGGGAGGCCAAGCCAGGGGGATCACTTGCACCCAGGAATTCGAGACCA
GCCTGGGCAACATAGTGAGACCCCCGTCTCTATTTGAAAAAAAAAAAAGAATTATGCCTAATTATATGGAGGAAATCCTAGGCCAAATGACACT
GGATATTCTCTGCTGCTGCTAGCAAACTTCTGTAAACCAGGAAGGGCCTCTCTAGAGAGCCAGATGAGAGGACATAGGGTGGGTGTGAGGAAGA
GTGAGTGCTACAAGACAGTGTGAGGAGAGGTGAGTTCAAGGTAGGGGGAGGACAAGGGAGAGGGTTCCATTTTCAGCAGTGGGGCGAGCAGCAC
AAAACTGCAAAATGAACTGCAGATGCCAAGGGCAGTACATCCTCTAATTGGGGTGAATATTACAGAAACGGTCTTCTCTAAATTTCCCCAAGCT
GCAGGAGGCATTTCCTATTACGTTCTATGTTACTGTTTTACCAGGAGAAAGGGTGGTGCATTTTACCCAGAAACTAACATAGGCTCCAAGTGGA
GGAGAAGATACGACCAGCAAAGGATGTCAGTAACTGCTGCAGAGCCCTGCAAACCTGAACCCTGACGCTGACGCTGCCCTTTTGTTTGGGATGC
AATTTTAATATTTCCCTGCTCCTCAGCCCTCATTAAAAGCTATTACAGAAAGAAGCCCTTCAGGTCTAGGTTTCTTTCAGATACAGTAAGGCTT
CCCCTACAAAGACCAAGGCATAAACACAGCCCTGTGACTGTGGTTTTGACACGTTAATTACAACACTTCATCATGTTATTAGTGCTGGCTAGAT
GTAGATTGTGAGAATGAGATTTTAGTGAAGATTTAATTTCTTGGAATTAATTTCCCCTTGTGTAATAATCTCCCTTCTTGTGAATTTCGTTTCG
GCAGGAGTGACACTCTTTGTGGCCCTTTATGACTATGAAGCACGGACAGAAGATGACCTGAGTTTTCACAAAGGAGAAAAATTTCAAATATTGA
ACAGCTCGTAAGTTTGGAGAGGGAGGAGAAGCATGTATACCTTTCAGGTTGGGAAGGGGTCTTGCATTTCTGGTCTGCATCTCTTCAGATGTCT
GAAATGCTAAAAGAACATTTGCTAAATTCACTGGTTTACAACAATATACTTTGAGAAAAAGAAGAGTCAGTGACACAGTGCAGTATTTTATGGC
ACTGTGTGTCCAAGGAGATTTTTGGAGAGTACCAAAAATACTAGCACAGTCCATGTTTATTGGTTTTGGTTTAGGTTTAAAGGTTGTAAAATCA
CCAGCATCGCTCTCCACAGCCCTGAGTGTTTCTACAAACCTACAGTTTCAAGTGATTGGCACAACCCTTGGCTACCCACGTTTCGTGCTCTCTG
CCTTGAGATTTTCTTCACATTTCAGATGTTACACTAGCTAAGCCCTCACTTGAGGCTTGCAACAAGTTCTTTATTTTATAAACTGCTAAACATT
TCTTTGGTGAAGCCTGTGGAGGCGTTTCCCTATAACAAAAATTTGGGAGTGACTGGAATCTTTTGGAGCTAGAAGAAGTGATTTCACCCACTCT
TGAATGAATATGCTGCATGGTTGTTTAATGTTGGCCAGGAGGAACAACAGTTAAACAAGCAGTGGGGAGATTTCAAGAGTGAGGATCACTCACT
TTTCAAATCCTACATTCAGTACCTCCATAAGAAGAGTAAACACGGGTCAGATTTTCTTAGGGTTTATAAATACTGCCTCTATTAGATTGAGGCA
GTGTATGTGGATGAGGCCCTTCAGAAAAAAATCTAGGTTCTGAAGAAAGTGTGGGGGGGTGGGGTTCCACATCAGCAAGCCAGGCATCTCCAGC
GTCCCTATGAGTGCTCCCGGTGGTTGGGAAAGAATGCCACCCAGGGGCTGAGGGGCACACCAGGAAGTTCTTCTATGCCACCAAGTCTGGTTGC
TTCTCCTCTGCTCTCTGAGCAAATCAGATTCCATGTTGGCAGAAGGACTTGGAGCCAGGCTCCTCAGCTTTCCCAGAAGGATATGCCAGAGGGA
GTAAGAGGCAGCCCTGGTCACACTGCACATCCCAGGAAAGAGGAGTGCTGATTGCCACAGGGACCTCCTTCAGGCCTGAAAACTGCCTCCTTGG
AAAGCCCACCCTTTTCTTGAATGCCCCCTGGCTCACAGAACCATGCAGTGAGAATATGCCGCCTTTCAACAGCAAGAACTGAATACTGCCATAA
AGGGCCAACGACTCTCTTCGAGACATCAGAAGGCACAGTCTACATAGCCACAGGGTTTTCAAACATATATTAGGATTGTCATCATTAACAAAGG
TGTGGGGTGGTGTTTTTTTAACTTTTTGTTGGAACACATGCAGAAAAGTGCACGTATCAAAAGTGTACACAGTGATGTTGCACAACAGTGTGAA
CGTACTTACTGCCGCTCAACTGTACACTTAAAAATGATTAAAATGATAAATTTTATGTTGCCTATATTTTACCACAATTTTCAAAAAGTATACA
GTTTGCTGAATTTTCACAAGCTGAACATGCCTGTGTGGCCACCAGCCAGGTCAAGAAGAGTGTTACCTGCCCCCCAGGAGTCCTCCTCCTGTAG
CCACAGGAAGTTTTTAAAAAATAATAAACAGCTACCATTCATTGAAGGCTCCTGATGTACCAGCCATGAGCTAAGTTACAAACATTCTCATGCA
TTCTTCCACTACTGTCTGAGGTATGCATCGACGTCATCGTTTGATATCTGAGGAGCCAGAGGCCCAGATTAGGAAACTGCCCCAGAGCACAGAG
CTGGCAGGTGGCAGAGCCCAGGGCGAGGTGCAGGGAGGCATCCTGAGAACAGTGTCAGGTTTGATCTGGGAAAGGCCCAGATACTTTATCAAAG
GGGATGGGAACCTTCAAGTGACAGGAAGCTTTGGAGAGTTGACTGGGGCTGGGGAGGAGGGGATTGGGCTTTGATCTCTCTGGGTATACCTCCT
TGCTGTTATTTTGCTGCTTGTTGGAGTCCATTAACTTTGAATTCTGAAATCCTCCGCCCCTTAGCCTCCCCTACATGCCAAGGTTGATTAGAAA
TTTCAAGACTATTTGTAAACTTTCAACTGCAAGAACCAAGGAATAGTAAGAGTGCAGACCCAGCTCTCAGGAGGCTTTGTCCACACTGCACGTG
CGTCTGTGCATTACGCTGGAGGGCGTTGGAAAGTCAGTCCCTGCCAGTGTCAGTCCCATGGCTTCTTCCTTTGTTAATCTGGAAACGATCTGCA
GTGTCACCAAATTGAAATTTTAGAGAAACACTTAAGGAGGATCCAATCCGTCTCAATCTCTATTTTGTGACTTCCATGCACGGCCTTTGATGTT
TACGGAGTCCTTTGAGCCTCCAACCCTGTTCTTGTAGAGGAAAATATTACAAAGGTTCTCTTCCTCTTAATGTTCTTCCTTTCTGCCAATGGAA
TTCCATATCAGTGGAATTTTAAAATTCCCTTTCTCAAGGTTATAGAAAAGAAAAATAAGGTTTGTGTAACAAGACAATATCCTGAAGTCAAGGG
TAATAACAGGGAAGAGAAAATCGTGAAGGAAAAACAAAGCAATAATACAGAAGCAGATGGCACACGGCTCCTCCCCTCCCCCATCCTCTCCCGC
CTCTGGCTCTGGTCCTCAGAGGAGAGGGAGGAATGGGGCTCTGTTCTGCCAGGGCAGCTGCCACACCTCCTAGTGTGTGTCCTCCCACTCCCTT
CCCTGGTGGAGCCCTGGTGGCGCTGGAGGCCACAGGGCTGGCTTTGTTAAAGTACAACATACACCGTGGGATGTGCATGTATGTATGTACCCAG
AGGCGGTGTTCCTGCTACCCAGCAAGGGGCAAAGGTGCTTCATTGACCCGTTGTAAGTGCTCAGTGTCATGTATTAGGACATTATTAAATTTGA
ACCCCTTTTGCAAAGTATTTAAGTTCATCCATTTTATTCCTTGTTTTCACATTTCAAGACTAGTCGTTTACCATCTACGAACAGTGACTCTTTA
AAATGTAATACATGTTTTACACAGAGTGGAGTATTGGTCACAGCTGAGGGTAGGTTTGTTGTTTTTTTTTTCCTATTGTGATAAAAAGAAATCC
TGGTTTAGAGCCCCACCCAAGCATTTGATAAAATTGTTACCGACATAAAATGCTACACTACTGAGCAGATCAAGGCTGACTTCTGTTGGCAAAT
AACTACTCCCTGGCTCCAAGGCTCTAGGGCTCCCAGCTCCGGGGCCCACAAGCCTACGAGCAACTTTGGGGTTTGGGGGTAGACACTTTTCTTG
GTGTGGGGTGTGGTGGGGGCACAGAATCTTTTAGCAAGTCTTTAAATCAGAGGAACAGAGACAGCTGTTATAAAACCTTAAAAGCCATTCTAAA
CCAGCGCCAGGCTTTAAATCATATATTTGAATTAGAGCAAGATAACAAAGAATATCTCACCAAGTGCAAACTGAAAAATCTGAACTATAAGAAC
CGTTTAAAGAAATACAGCTTTAACAATGCTAATTACCCACAGTAACTCAAGAAATAAAAGATAACATAGTAGAGATCCTATTTTCCTAAATCAA
CATGAATATAGAGTATATCTATTTATAGGTGAATTGGTGCTTCTAGTGTATCAAAAAGATGACTTAATTCACTCGCTCAACGGCTCTCAATGAA
TATTTTGAGTCTGTACCAGCCTCTCTAGGCTCAACATGTGAGGTTTACTACATAATAAACATTGAATCTTTGTCACTTTATGTCTCCTTTGATG
AAGAAGATGACCCTAGAAAGCCTAGAAAAAGAGAAATCAAGGATTTTATCTTTCTCATGAGTAGCTGAAAACCCTTGTGAGATTAATAGGGTCA
AGTGAGAAAATCATGAATTCCTGATTCATGACTTCAGAATGAGCTGATAATTGCATTCTGATCTGTGCTACACTATTTAGGTACCTAGACCAAA
ATGAGATAGTTAATGCTAGATTTGAATTGGAAATGACTTCATTAATGCCAGATCACCTGAATAAGGCTGGGCTTGTCCCAACCTGCAGCAAAAT
GTATTGGCTTCCCGCCCCTATTGACAGTCTCAGATACCCTAAAATTGATGCTAGCCATTTTTTTCTTAATTTAATTCAATTAGAACTAACTTGA
TGAAGGTTATCTTGCATTAAAAAACAATAAGCCTCTGCTACAAGCACTTAATTTTTTCCTCGCCATTTTGGAGCATTTCCCAGGCTGGGCGGGA
GAGGGTGGAGGGGGAGGAGCTGGGTATTGTTATTTTTAATGATGAAATGTCTCCATCTAGTGGAACTTCAGAGCAGTAAACCTTGTCACTATAT
TAAAGTGAAGATTATGTCTCCAAAGCCATTTTCCTTAAACACATACAGTGTTTAAAGAAAAAAATAAACTTCCACATATGTTCTTAGATGATAT
CCTTACAACTGTATGTATTCCTTTTGGAAGGCATAGAAATGAGACCAGTACAAACTGTTAACATTTTTAATGTCATGATTTGAACCTCAAAGAG
AGGGGTGCTTACAATAGGGGAGCAGCACAGTGCAGATCCCACCCAGTTGGTAACGATCTAGCTCTTAAGTTGGGCAGTGGGTTCACGGGGTTCA
TATATACTGTACCTATATTCTTCAGTATGTATAAATATTTCCTAACAAAGATCTAGACATGAACTAAATTTAAGATTATGAATAAATCTAGACA
TGAACTAAATTTTATGAATAAAGAACTTTCTGAATATGAGTACTGTACTTTTGCTGGATTTGTAATAGGGTAAAAAGGAGAGATTAGGAAACTA
AATTAAAGCATAAGGTTTTAAAAACCTTTGCTGGCACCTTGGTTTTTTTTGTGGGCTATTCTGTTACTTATGCCACCAAACATTTTCCCACAGC
AGCACCCCCAGCACATGCTGGCTGGTCACCTTTGTTCTGTTGCTCTATTAAATCCTATCTGGAGGCGTTGCTCACTGCTCACCTTGCTCAGAGA
GTCCAGCTTTAGAGAGCGGCCTCTGGAGTAATGCTTGAGTTTAATGTGGAGGCTGATTATTCTCTACCTGATTAAATCTGTCTGGGGAGCATCT
TTACCATGGCCAGAAGCTAACACATTTTTTTTTTCCCCTGGCTGAAAGCTTTGACACAGAACTCAGCCAAAAGAACGATTTTTTTTTAAAATGC
CTGAAGTTTTGCTTGCTGCCTCCAAGGACCAAAAAAGGAGGAAAAGAAGGTCCTGAGCCCCAGGAGATCAGATTTGCTGGGAGGTCTGATTTGC
TTGAGGGCAATCATGTCGTTGACTGCAGGCTGGTGGAAGGTTCTGCAGATACTCAGTGGATGTCAGAGCCGCAGCGCCACATCCATGGCCTCCC
CGATGTTAATGGGAAGAGGTACACACTCCATTCCTGCTCTTCATCACAGGTAGCCCTGTTTGAATCTAAGCAGATGAATTTGAACTGCTTAATG
TACTCACTGGCTTTAAGATGAGAGTAGCTACAAGATCCCCTTTCACCTCCTCTGAGAGCTCCCCAGATTGATACCACACTGCTGTGCTGTGGCA
TGGAGCCTAAGGCAGTACAAAGTGGCGTGACTAAAAATCTCACTCCCTTGCCGAGTTTCTGTGCTTCACCTGTGCGAGCACACGCAGCCATTCC
CATCCTCACCCCTTGCCCATGAGCCTCAGGCCAGTGCTCTGGACATTTGATGGAATCACTTCAGACTGAAAATGCCTGGGGAAGGTGATCCTAC
TGTAGCATCATTCTACCTGCAGAGAGGTGTCTCCCCATCTTTTTGAGAGCAAGCCAGGCACGTGTTTAGAGCATCCCTTCATCCCTCATCAGAC
CTCCTAACCTCCCAATGTTAAGATGTTAACATTTCTAATGTCATGATTTGAACTTGAAAGAGAGGGGTGCTTACAATAGGGAAGCAGTACTGTG
CAGCTCATATTGACCTCAGACCTCCTAAAAAGCTTCCCTTCCCAGATGATCTGCAGCTAACATGTTCTGCTTAATTAGGCTGAGGCTGGATTCC
TTTTCCATCCATCCAAGTTAAGGACACTAACCAAGCAAAATGCCCCCTACCAAGAGAGACTTCTCATGGCCAGGATTCCACATTCTTAATTCAC
AAATAAGGCAAGAGACAACTGTGGGTTCATCTCATGGCTGACTCGAACTGGAACCTTCTGACCTTCACAGGGGGACTATGTTGGAGTTCAGAGT
AAAGGAATAGGTTGGAGAAATCGGGTGGCAGGATGATTAGGTGACAGTTATACACTTCAGACCACAGTGAGTCTCTAACTGCAAGCTGTTGAAA
TGGTCAACATATTTACTTTTCTTTTTTATTTACAGGGAAGGAGATTGGTGGGAAGCCCGCTCCTTGACAACTGGAGAGACAGGTTACATTCCCA
GCAATTATGTGGCTCCAGTTGACTCTATCCAGGCAGAAGAGTATGTTTCATTTTGTTTTCACTTAACTACTTGATTTTAAAAGTTGATTGCCGC
AATAAAATGCCATCAGCATTGGGCAGGAGGGAGAAGAAATTCACAGCAGTGATTAAGGCTGAGTTTTTCAGGCTTTCTGCCCCGGTTTTTACCC
TGTGTAATACTTGCACTATTATTAACTTAATGTTTCTCTTGAAGCCTGCTCACTTATTTTGAAAGAAAACTTTCTATCAATACCCTAAATGAAA
AGCCAGTAAAACTTGTCATAGATTAAAAGTAGCCATAAAAATATGAAATGCTAGCTAGAGCATATATTATTGCCAACCCAAGTTTCTAAGCCCG
CAGTCCACTCCCTCTGTTGGGAAGAGAGGGGAGAGTAGCAAGTAATTGCCACAGATTAAACTAAGATTTTTCTCCTAAGTGTGATCAGAAATAT
TAAAAGAGTGCTGAAAAGACACCTTCTATGCCCGGTAAAAGTGACTCACATACAGTGTGTCCCCCCAACTTGGGAAATACTACAGTGATTTGGA
ACCTCAGTTGGCTTCTTTGGTAGTAAACAGTGACGAGCAGTTATTTGGCCCAACTATTCAACTGGTAGCAGTTTAGGAACGTCTTCAGTAGCCT
GAAGGGATCTAAAAGGGATAACCTTTCTCCCTTCTTCTGACCCATGAGGATTAACAGGCACTCCCAAGGGGAAGAACTGCCAGTGGCTGTTTGC
CAGCCCTGAGGGAAGGAGAGATGGAGCCATGATTTCCCAAATGCAGGAACTTGGCCTCCTCCTGACACCTTGGGTCTTGCCTCTCTAAAAAGGA
AGTTGAGAAAAGTCAGTCACCAAGCCCAACCTTTGGCTCTGCTATTTAATTTCACCATGGATTTGTTTAAGTAGTGCAAGAGGGATTAGACTCC
ACACCCTAGCCAAATCCCCAGTTGGATTGTTCTATTCTTTAATTCCTTTGTATTTCCTCTAAGAGGATCTGTTTTGTTTCCCACTCTCAAATAC
TCAGTGGTTCTTAGTTGTTTCTTTCTCCTAGTGACTGTGTTTCGTCATCAGTGAATCAAGGTATTTGTATTTAATTTGTATTTTAGGGCAGATT
TGCAAACATCTTTGTGTTCTTTCAGAATAAAAAGTACCACCAAAATGTAGCATTACCCTGATTTAACTGGGAATTTGTCAGCTATATATTTAAG
TTATTTTTATAATGGTCAACTTTTTAAATATTTTGTTTTTAAAGAATATTATTTTTTAAAATAATGCCTTCGGTTTTACATATGCAATTAACTA
TCATTATCTTAAAATACTATTGTTTTGGTGCAATCCATTGTTCTAACAATTCCTTTTTCACCACTGTTTTACACGGCATTCTAATTGGGTTATT
GGTACAAACCCAGAAAGCCATTTCTCACAAATAATTGACGTACTAGTCTTTACGCCACCTAATGCTAGATTAAGAATTGTTGCACATTTTGGTG
TTTCTAAAAGTCACTGAGGAGCTAATACGCAATGTCTCTTTTCTTCAGTGAAACTTAATTCACAAAAGAATTTGACAGTGAACTCATGATACCT
ATATTCAAGCATAATGAATTGAAATTTCATTTTAGAATCTGGATTCTTTCACAGTTAAATATTCTAAAAATGGTTCCCACACACAGATTCCAAT
TGAATGCTCCAGCATTCTCCCTTGGGTTTTGGGGGCACCTGCTGCTCATTCAGCACCTAGTGCTGAAATAGTAACTAATTGCCAGGCTTGGTTA
AGGCTTGAAAGTGCAGGGGGAGGATATAAGGCAAATAACAAAAGTCTTGAGGTGACTTAATGTGATGCCTCCTAAAGTGGCACAGAGTATCATT
AGTTTCTTCAGGGATAATATATTGAAATATCTGCAACACCAAGTCATTTTAAGGGCATGCTTTTTGACTGGGCACAGATCTCAAAGGTGTACAG
TAACCAGTTCTTACAGTTGTGCCAAAGAAAAGATGTACAGGGTCCACCGTGATGGCTCCCTCCTGTAATCCCAGCACTTGGGAGGCCAGGGCGA
GAGGATCACTTGAGCCCAGGAGTTTGAGACCATGCTGGGCAACATAGGGAGACAGTCTCACTCTGTCCCCAGGCTAGAGTGCAGTGGCCCTGTC
TCGGCTCACTGCAACCTCCACATCCCTGGTTCAAGCAATTCTCGTGCCCCAGCCTCCCGAATAGCTGAGATTACAGGCACATGCCACCATGCCC
AGCTAATTTTTGTATTTTCAGTAGAGTTGGGATTTCACCATGTTGGCCAGGGTGGCCTCGAACTCCAGACCTCAGGTGATCTGCCTGCCTCAGC
CTCCCAAAGTGCTGGAATTACAATCATGAGCCACCACTCTTAGCCTCTACAAAAAAAATTTTTTTAGTTAGCAGGTGTGGTGATGTGCACCTTT
AGTCCCAGGAGGCTGAGGTAGGAAGATTCCTTGAGCCTGGAGAGGTCAAGGCTGCAGTGAGCTATGATTATGCCACTGCACTCCAGCCTGGGTG
ACAGAGTGAGACCCTGTCTCAAGAAAAAAAAAAAAAAAAGAAGAAGAAGATGATGAACACAGATCACAATCCAGGTGCTTGGTAAATAAATGCC
TTTACTTATCCAAGTCCTCACACACCTAGGGGAGGAAAACAAAATACAAGTAAGTACTCATGGGCGTTTTTCTCAGAATTCTTATAGGGAGGAA
GTTATTGAAAATACTATTCCTTTATTCATATCATATCTATCTAATCTGACTTTTTTTAATGCTTGATTATCTTCATATACATTAAGACTGTGTT
TTGTTATTTCCCAGCAGTTACAATAATGCTTAGATATCAAGAGATTCAAGAGTTTTGTTTTTTTGTTTATTTTTTGTTATGAGACAGAGTCTCG
CTCTGTTGCCCAGGCTGGAGTGCAGTGGCGCAATCCTGGCTCACTGCAACCTCCACCTTCCAGATTCAAGCAATTCTCCTGCCTCAGCCTCCTG
AGTAGCTGGGATTACAGGTGCATGCCACCACACCCAGCTAATTTTTTGTATTTTTAGTGGAGATGGGGTTTCACCATGATAGCCAGCATTGTCT
CCATCTCCTGACTTCATGATCCACCCACCTTGGCCTCCCAAAGTGCTAGGATTACAGGCATGAGCCACCATGCCTGGCGTTTGTTTTTTTTGTT
TTGTTTTGTTTTGAGACACGGCTTTGCTCTGTCACCCAGGCTGGAATACTATGGCACAATCTCAGCTCACTGCAGCCTGGGCCTCCCAGGTTCA
AGTGATTCTCCTGCCTCAGCCTCCCGAGTACCTGGGATTACAGGTGCCCACCACCACACCAAGCTAATTTTTGTATTTTTAGTAGAGCTGGGGT
TTCACCATGTTGGCCAGGCTGATCTCAAACTCCTGACCTCAGGTGATCTGCCCACCTCGGCCTCCCAAAGTGCTGGGATTACAGGTATGAGCCA
CCATGCCCAGACTAGAGTCTTTTAGATAAGTGTGGAAAGGGTCTCCTGCAGTAACCATTTCAAATCAAGGTCTTAGATTTCTGGTTTTTTGCAT
ACAGGTGGCAGTTTTAACACAAACCAGTTCTGGTCATGTCTAGAGGGGAGGAAAACAACCCACAACTCAAATCTGATTTTTTTTAAAGAGTGAA
AGAGATGAATTAGGGATAGAATCCAGGATAGATTGAAAAGGAACTGAATTAAGCTAGAAAAAAAATACATCCAGGGAAAGGTTAAGGACTAAAT
TGGAGGAGAGGGGAATTCATGGGCAAAAAAAAAGCCTATGCTGTTGCATTTTGTAAATGACTATTTCAAAATATTTTCTTTCCTTGATCTAATT
TAAAGGTGGTACTTTGGAAAACTTGGCCGAAAAGATGCTGAGCGACAGCTATTGTCCTTTGGAAACCCAAGAGGTACCTTTCTTATCCGCGAGA
GTGAAACCACCAAAGGTAAGATAACGAGTTGAAGAAGTTGGCTTGTCATTCAAATCTGTGGATTAGTCAGAGGGGATGAAAGGCTTATCTCTAA
TGTACAAGGATTTTAAAATGGCACATACCCTCCCTGCCTTGTGAGTGGAGTCCTGTTTTCAGTCTTCCCTCCAGGCTCTAAAACTGCAGACAGC
ACCAAAAGAACTACAGAGGCCCCAAATCAAGACCTTCCTCCTGAAACGGGCTGGATGCAGCTACACTGCTCCAGGCACACAAGATCGTAGAATT
TTCAGAAGTCCTTTAGAATTTTGATTTTCTGAAATCTTAGTTCCTCAGGTGCCAGTCATACAGGTTGTTTTTGCTTTCCTTTTGTTTTTTAAAT
AACTGAGGACTTCACTTCTCTTCCTCCAACTGTTGGACTTTTCATTATTTAACAACTCCTTACAACCCACAAGAGGAGACAGGTCAAGGAAGAA
AAGAGTAGTGGCCTCTACCTTAATTTGCCAGATTATCCATTCCTGTAAAAAGTGTTGTAAAGCAGAAGCTTGAAGCTCACTTCTAGAGAAACAA
GCCTTAGATTTCTTTTGCACATGCTGCTGCTACCTCCCAGTATGCCCCTGGGTAGAGAAGACAAGGGAAGCAAGCTCAGCCATTCATCGTGGTC
CTTCTTTACCCTTGGGGGAAAGGAGTGTTGGAATTGAGTCTACGCTGTATTATGCAACAAGGGGTACCGAACAGTGTGTTTAAGAAAAATGTCC
AGGAAGCTTTCAGTCTCAAGGGCAGAATCATTTTATTGCTAATATATTCTGACATTTGCAGAACATCAAGAAGCATCTTCTTCCTGTTACTGAG
TGGAGATGCTGTGTGTGTGTACATGTGTGTGTGCATCCTTGGTAATTATTAATCAGAGTCAAGAAAATGAGGCCTAAAAAGCAAGCTGATACTC
TAAATGATTGGAGCTGCTAATGCTTTCCACGTTTACCAGGTGCCTATTCACTTTCTATCCGTGATTGGGATGATATGAAAGGAGACCATGTCAA
ACATTATAAAATTCGCAAACTTGACAATGGTGGATACTACATTACCACCCGGGCCCAGTTTGAAACACTTCAGCAGCTTGTACAACATTACTCA
GGTAACCTTAATTCTAACTACCTACTAACCATTTGGATGCTGGAGGGAGTGGATAGGCCCAGAGCAGGAAAGTGGAAAGGGGTAAAACTAATAT
GTACAAAATCACTTATAGAGTATGTTTTCTTTGCCAGCCCTTAATGGTAGGGCTTGGCTGTATAGGTTCTATTGCTATTGTTCTACTGTTGTTA
TTATTAGTTCCACATGTTTGTCCGAAGATTCTATAATATTTTTGTTTCCCAAGTAGAAAGCAATAGGTTAAATTTTAAAAGTTGTTTGTTTTTT
CATCTTCAAGTTGTCTTTTCCCATTCCCCGCGTTGCATGTGAGAAAGCAGTCTCTCGGAGGGCGCTTACATGGGGACTCCTACAAGGAATATTG
TGTTTCCACTATCACTTTACCCTCAGCACTGTTGCAGGAATAGCTGCACGTACATTTCATTCTCATGCAAGCCATTTATCATGTTCCCCACCAA
TACTGCTGTTTCCTAACTAGGCTTAGACTTACACAACCCACAGCTCTTAGATCTATTATGTCTATTGTGTTAAATTAAACTTTTATAATGTACA
TCAGTTTCTGTCCTAACACCCAGATATCACCATTATTTTGTTCTTTCTGAAAACATATAATTATTAGATATGCCAGTTTATACATTTTATCAAAG
ACTCGTTTTATGTGCAATTGTTAAAATGATTGCTTTCTTTCCTAGATTAGCAAGTATTATATGCCACTATATAATACATGTTCACATGTGCCTA
TAGATATAGGTGCACATGTATAGACACATGTCTATATCCTATTAAGCCTAGAGATATTTTTTTGTGTGACCCATTTCTCCATTTTCTATTGTTC
TCCAGGACAAAGGCAAGACTCAAAAATAGTATATGATTTACCTCTACTATTGCTCTGTGCACAATCATTGAAATATGTATGTATAATTTTTGGC
ATGAAAAAGCAATCTTTCGAAAATGACAATAAGAAATGTCTGTTGATCCCTTGCACTAATGAACAATAGAAAATATTAACAGCACTTGCAGTAC
TAGTTGATTATGGCATTGGCTAACTCGGTTTATGTGGATTCTAATGAAACATTTGGGAGAAATGGAACAAAATATCTCCCTGAGCTTAAGTGAT
ATACTCAAAATGCCTGCATGTTGTAAGGATTTTTCAAATGCTGAAATATATTTCATTACAGCCAAACATCAGCAGGATCCCCAGGGTTTGGATC
ATTTGAAAATACTGTGTTCGAAAGGGCACCTTAATAACATTGTAAAGCTGGAAGGAAAGAGGGGATATGAACAATAGACACTTTTTTTAATCTC
GGGGAAAAAAAATCCATCTCACGTTAGACTTGTTTTTATATTGGCTTTCCCTCTTCCCTCTTTCTCTCTCACACAATAAAATGCCTTTGAAATG
CTGACACATAAATAATTTCCTACCTAAACATCTCAGAGCCTGATTCCATCAAAAAGAAAGGAGTAAAAAGCAAGTTACAGCCCAGCAGCACATC
TGCTTTCCCTGGGTCCGGGGGCTGCCAGGAGGGAGCGGGAGGTCTGTCCACCTCACAAGGCAGGCTCTGTCAGCTTTTGTCACTCCCTGATTTC
TTATTCTTTGTTACCTTTTTTCGCCTGACTGATTTTTACTTGGCATTTAAGTTCCCCTTAGCACTGCCAGATTCTAAAAGGTTATATTCTTTTT
AAAAAAGAAGAGAAAGAAAGAAGGAAAGAAGACAAAGAAAGAATAAAAACCTCCGAGTGTTAACTACTTTTTCCTTTCTTCTTTTTTTTATAAA
GAATACATTCTTTCACATCTTGAATTTCTGTGAATTTTAGTTTCCATTCTTTCTCCTTTCAAACCAGACACCTAAATTATACGTCGAAGACTGT
TAAAAAGTTGTTTTTTTTTTTTTAATGGAAAATATCCAAGAAGCAGCCCAGGAGTATCTGACATGGTGGAATGGAATCAGTTAGAAAGCGAAGA
AATCACTAAAAAAAGTTACTTCTTTTTTTCCCCACCAGTTATAATCTTCAACCTTACTAGTTTATAACAGTTTAATGTCCTATAGAAGGATCCT
CCACTAAAGTTATAATTTTAAGTATAGTCATATAGAGAGATCCCTAATCCCCTGGGTAATCTAGATACTAAAGGTGGGGAAGAACAGTCATATT
GACATTCTTTAATCCAAAACCACTGTTTGAAATTAGTAAGGATATTTTCAGCATTCCCAAAAACATGTTATTAGCACGTTGAGCTGAAAACGTT
TTTCTTCCTCAGTGAGTACAGAAACCAAAGCAGTCTGCGTGTATGTCTATGTATAGACTGTATCGTACCTGGGCTCATGGAGTAGTCTAAATTT
AAAACGTCCTCTCTTCTACCTCCAATGAAAATGTTTCCGTGTGTGGCGTCTGATCTTCCACCGTGTGTCTGGTCGTCTGCTGGTGTAGCGCTGT
TTAAGGAGCGCTGTGTGCTGCTAGTGTTCCACGATGTGTGTGGTCGTCTGCTGGTGTAGTAGCACTGTTTGAGGAGCACTGTGCGCCGCTAGTG
TGGGTTTACACTTATGAGTGTTGTCATTACATGTGTTCTGCTCTTCTCTCCCTCTCCTGCCCCTGCCCTGCTCCATCAGAGAGAGCTGCAGGTC
TCTGCTGCCGCCTAGTAGTTCCCTGTCACAAAGGGATGCCAAGGCTTACCGATCTGTCTGTCAAAACCAAAGATGTCTGGGAAATCCCTCGAGA
ATCCCTGCAGTTGATCAAGAGACTGGGAAATGGGCAGTTTGGGGAAGTATGGATGGGTATGCTGAGACTCAATTACTCTCTTATTAGCTTCCCC
GTTTGGAAGATCCCAAACACCAAAGATGGAAGGTGAAAATAAAGACTGCGTGACCGGGAAGAAAGTTTGAATTACTAATAGTGGGGAATAATAA
TTTCAGTTTTGGTTTTAACATTCTGGAATTCCTAAAAAAAAAAAAAAAAAAAAAAAAAGATAGTAAGTGGGCAAAATTGGCAAATGGCTCAAGT
TGTACTATTACATGACTCTCAATTCATAAACCAGCAAATGGTCCAAGGAATGTTCCAGCTGCCATAAAATATCTTGCTCTTCATTTCCAAAAGC
AAATATTTGTTGAGTGTCATCCTAACGGGCAAGTCACCTTCCATCTTTCCTAGTGCATGCATAATTATGGATTATAAGGACTGTGTTTTCTTTA
TTAATTCCCTTTCCTGTAGAGAAAGCTGATGGTTTGTGTTTTAACTTAACTGTGATTGCATCGAGTTGTACCCCACAAACTTCTGGATTGGCTA
AAGATGCTTGGGAAGTTGCACGTCGTTCGTTGTGTCTGGAGAAGAAGCTGGGTCAGGGGTGTTTCGCTGAAGTGTGGCTTGGTAAGGCAGAGGG
CACATTTTGTTTTGGCTGCATCTCAAAGCCGATTAAAAGGATGGTTTTGAACAAAAATGCACACACAGCCGTTGGGACAAAGACACTTAACCTG
GATAACTAGACGGGCACAGGCCATCCAGATTTTCAGAATATGCTGATACCTTGCTGTGCTCATCTGTCCTACTTCATGTGGTTAATTAAGGCAC
CCGCAGAACAGGAAACCAGTACACTATGAATCTGGTGTTTCCTCTTCCTAAGAAATGGGAAGGTAAGATACTGTATATGGGCAGCCATAAGAAA
GACCATGGGCGTTTTGCTCAATGGGCCTGGGTTTGAATCCTTACTAGCTAATTAACCACGAACAAGGGGCATATTTAACTCCTTTAAGCCTCAG
TTTTTTCATCTGTAAAATTGGAATGATACCTTTTTGTTGTTGTTGAGACGGAGTCTTGCTCTATCACTAGGCTGGAGTGCAATGGCGCGATATT
GGCTCACTGCAACCTCCACCTCCCAGGTTCGAGTGATTCTCCTGCCTCAGCCTCCCGAGTAGCTGGGATTACAGGTGCCTGCCACCACGCCCAG
CTAATTTTTGTATTTTTAGTAGAGACGGAGTTTCACCATGTTGGCCAGAATGGTCTCGATCTCCTGACCTGGTGGTCTGCCAGCTTCATCCTCC
CAAAGTGCTGGGATTACAGGCGTGAGCCACCACGCCCGGCTGGAATGATACTTATTTAAAGGATTATTTTTAGGATTACATGGGTTAATATGTT
TAAAGTCCCTTGCAGATAGTAGATATTCAGTAGACATACATACTTCTCTTTATTTGATTGTCATTGATAAGGCTGGCTGCACTGGGCCTTTGTA
ATTTCTTTTATAGCCCTTTTGGGTCAGTTACAGCTTTGCTTAGAAACAATTCATGGGTTTAGGCCCCACTCAAAAACATGTTTCAGTAAAACAA
TATTAATACTTATGGAATCCTCTATCTTCCTTCCTCAGAACTCATTAAAAACAATATCGATCAGTACCCTTTGCTACGTGTGGGTATAGATCTT
TAGGTGTGGTCTGCATTAAAAGAGTTAATACAGGTGGCCGGGCACAGTGGCTCACACCTGTAATCCCAGCACTTTGGGAGGCCAAGGCAGGCAG
ATCACCTGAGGTCAGGAGTTTGAGACCAGCCTGACCAGCATGGAGAAACCCTGCCTCTACTAAAAATACAAAATTAGCCGGGCATGGTGGTGCA
TGCCTGTAATCCCAGCTACTCAGGAGGCTGAGGCAGGAGAATCACTTGAACCTGGGAGGTGGAGGTTGCGGTGAGCGGAGATCGCACCATTGCA
CTCCAGCCTGGGCAACAAAAGCGAAACTCCGTCTCAAGAAAAGGAAAAAAAAGGAGTTAATACAGTTAAAGCACTTAAAACATAGCCCAGCAAA
TGATAAGTACTACAAAGATGTATGTAGGTATGTTAGGTGGATAAATAGGTAGTAGACAGACATACACATATATATGTATGAGTTAGCTAGTTTA
ATAAATCCACATTATTGTGTCCATCTCTCTCTGGACTTAATCTCAAGTATCGTAAAATATATGACCTCCTTCTTCCTCATTCACCCTCTAATAA
TAGAGGATTTTAGTCAAATTACTCACACAATCACAGAGAATTGGACCCTATTAGAAAATTTATTTTCAAATTTTACAAATATCAACTTTAAATC
ATTACATATACTGTTCACATTTTGTTTTCCAATAAACACAAGACAACATGCCCATCAGAATTGGGATGGATGTCTGTGTAATTGACGAAGTTTG
TATTACAGAGGAAGATCTTAATGTATTTAGAATTTTGGGTGCTGACTCTTAAATAGCAAAGATCACTTATAAATTGTCAAATTTCCCATGAATG
CTTAGTGGGCAGATATTTTATGAAGTTTAAATGAACTTTTCTTTAAGCCACTTGTTTTTTCATAATTTATATTTTGTAGGTTGCATGTTGTAAT
TGTATGAACCATGTCTTTAACAGGATAATAAGAACTCCAATTTACAGAATCCTTAAATTCCAAAGTCAGTTATATTAAATATAAAAATCTTAAT
TTAAACCAGTCTTCTTTTTCAAATAGAAAATAATCTAAATTAGCAGATTTCTTCCCCTTTATAAAGTTATTCTTAAGCATCATAGGTCTAATAA
TGGAATACACAAAGAACCTAGGTATGGTTTTGTTTTTTGAATGCTGGGGCTGAAAGAGACCCAAAAGATAATCCAGTTCCATTCCTATATTTTA
CAGATGAGGTAACTGAGGACAATCATAATCCAGGCCTTGCCTTCAGATTTGCTAGATTTTTTTTTAACATGCTAAATTTAAATATTTCTGTTCC
CTCTCAGCTGCTGTTTAATTTCCCCTGTCTACCTACCTGACAGAAGCCATTGTTCTTGTGGCTAGCCATCAGTAAAGCTGAGGAAATTTGCCTT
ACCTTGTTGAAGTATTGGCAAGCAGACTGTGAGCTCCTTGCCAGTGCTTATCAGCATGCTTGGCACACCTGTGCAAGGACTCAGTCAAGTAACT
TGTCCATTGCAGACATTGAAAGTCTAGAGGTAATTTCCAGACATCCTCATGTGGTGCCATATTTTTAAATGGCACACCCCATAAACTTAAATAA
TCTAATTTCTTCCATGCACACTGTACTCCCTAAGGGACAGCTATTAGATGGCCAGGCCTGTTCCAACCCCTGGACTTTCATCACTGTATGGTGC
AGAGCTTTTGGTATAAATGTGTAATCATATGACTGAGCCAAAAAAAGAGAGAGTGGTAATATATCAAAATTTAAAAAAATTTTAATGAGTTATA
AATCTTAATATAGCCATCTTAGCCATAACCACAAATAAACTCATTTTTTCTGTTAAAATACTTGACAGAGTCCTTGCAATTGAATGTCTTTGTT
CAACAAAAACTGTATTAAGTGTTTTAAATTTAAAATCTAATCTTATGCAAATAGCTGGTGGTCAAAACCTTTTTCCATCAAAAGAAGATCTTTG
GTTTGACTTCATATTCTGGGATGTTTGTAGGTACCTGGAATGGAAACACAAAAGTAGCCATAAAGACTCTTAAACCAGGCACAATGTCCCCCGA
ATCATTCCTTGAGGAAGCGCAGATCATGAAGAAGCTGAAGCACGACAAGCTGGTCCAGCTCTATGCAGTGGTGTCTGAGGAGCCCATCTACATC
GTCACCGAGTATATGAACAAAGGTTGGGCAACACCTCTCCTGTCTCCAGCTCACAGTGCCTTAAGGGGTTGTTTAGGGGAGAGAAATGGAAGTT
TCCTACTTGCTACTTTTCTAGTTTCTGCCTGGGTCAAGTATTCCCATTAGGAACCACCAGTTATGTGCTCGTTAATGAAAACATTTTTTAAAAC
AGACTAACCTTGCGTTTACCCCACCCCAAATCCTTATGAAATGCCAAATACTTTTTACATGTTATGTGTGTCAGAAATTCAAGCTCCTCTCCAT
AATTAAAACATTCATTTTTAATATCAGGGTGGTACGGAGGGTTATGATAGTTTGCTGTTATTTTGAATGTAGTAGCTTTTGTTACATTTCCTTT
TTGCAGGGCTAAACAATTAATATCAGACACTGGTCTCTAAGCTAAGTTTAGTCTCCATCATTTCAACATCTTTTTTATTTCCAGCTCTCTTCTT
AAACGTCAGGCGATGAAATTACATGCTTTCAGAGACCACATTTATTCTCACATTCTCTCCCCTACAACCCCTCTGTCTTCCTAATCAAACCAGC
ATTGTTAGGGATAAGGCCAGAGATCATTTTGTTTTGGAAAAATAGATGAACCAAACAAGAAACAGCCTTAAAAGGCAATACATTGTTTATCAGA
GGATACCAGTGTAATTTTTCTTTTCCATGTACTTTCTTTTAAAATTTAATGGGAATTTTTTTTTCTGGCAAACGGTTTGTCCTTTGTATCCAGG
GTTATTTTGTAAATAACTTTCTGATTAAAGCATAAAATATCACTGATGACCATCATAAGCCTCCAGCTTCTGCAAATGATTATCCCTGCTTTGG
TGTTAGTTCTAAGCCTTCTTAGGCTGTGTTATAATAGTGGCTCTGAAGCTTCCAATGTTTCATTTCAGATGTCTGATTTAAGGCTTGGCTGGCT
GTGATCTAATTAAGGGGAACCATCTGCCCCACCCATATTCTTCAGATTGTCTTTTCTTGATTCAAAATCTGTAAATAAGCTAAGCTAGTGAGTC
TCAAACTTTACCTTGTATTATTAGAATCACCTGTAGGCCTTCTTAAAACCCAAGTTTCTGGACCCAACCTTCAAAGTTTCTGATTTACTCGATC
TGGGATGGGACCTGAGAATTTGCATATCTAACAAGTTCCCTTGTGTTATTGTAGCTGCTGGTCCCAGGACCACAGTTTTGGACCACTCAGCTGG
GCTGTATTTTCCCCACATCAATTCTTTGTGCAGGGGTGATTGGGGAATGGGATTGAAGAGGTGCAGTTCTTCATAGGTTCCTCTTTATGACATA
CAGGGAGATTGAGTCCCATTTCTTCTAGTTTTATCAAATATATGTTGTTATTCCTAGAAATGGAATTAATGCTGTTTGTTTTTAAAAGGTAACT
GTTACTTCTCCATGCCTATACTTTTTTCTACCCTGCTTTTTTATATAAACCTTTTTGTCCCCCCATGTGGATGGCATTATTCCATTTGACTACC
TTATTACTTTTAATAAGAAAACCAAATACAGCTCTCTGTTGGGAATTATCTTTCAAAGTAAATTGAAATGATTTTATGTTTTGTTCATAGGAAG
TTTACTGGATTTCTTAAAAGATGGAGAAGGAAGAGCTCTGAAATTACCAAATCTTGTGGACATGGCAGCACAGGTAGGCCCTGCAGGGTAACTT
GATGTGCCATAGATTTAATGACGTGTCTAAGTACATTGGGTCGTGGTGTAACATGTCCTGTTCCCTGGGTTTCCACAGGTGGCTGCAGGAATGG
CTTACATCGAGCGCATGAATTATATCCATAGAGATCTGCGATCAGCAAACATTCTAGTGGGGAATGGACTCATATGCAAGATTGCTGACTTCGG
ATTGGCCCGATTGATAGAAGACAATGAGTACACAGCAAGACAAGGTGGGCACTTGAATGACAATGGCTCGTGATCACAGCTGTGAGAACAGTCA
CTTACTGCACAGCCCTTCCTTCCTCCTTCCCTTCCCTTCCTTCAGAAAATACTTGAAATGTCATTCTTCAGCTTGGTTTGGACACCTTGATCTG
GCAGGAATTTGACAGCTCTGCAGGTGGGAGACTGTTGTGGGCCCAGTGGAGGGATGAGATATTGGTTTTTGGTGGGTGGGTGGGTATTGGTATT
TGCTGCTGTGGTCCTCTTGGCTCATGCTGGTCTCCTCACAGCGTCCAGGCCTAGAATACTCTGTTTTTGGAGAAAGAGACCACATTTATTCTCA
CATTCTCACATGCCTGTGTTTTTTACAAAGGCACAGGCAGGGTTTCAGAAAGCCAACAGGGCATGGTGCAGTGTCTCAGAGCTTGTAACAGCAA
CAGAACTAAGGCCTCAAAGGTAAGGGGGCAGAGAAGTGGGCACAAGAAGTCAGAATGGGCCACTGCACAGAGAGGGCCTCCTCAGTAAAGAGAG
ACGTCAGGGATGGATCTGGGGATTCACACCCAACCCCACTCTAGCCCACTTCTGGGTCTTCTGCCAGACCCAACCAGAAGTAGAGGTCACAGGA
GCTCAGCGATGAATCCTTATGGGTCCAGAGCAGGGAGAGGGAGAGTGGAGAGTGGACTTACACTACAGATGGTAACAGCATAGTTACTCGAAAA
TCTCTACCTCCTTTCATCATCCCCGCCATCCCACCCTCTGCAAAAATTTATAGAAATTAGCCTTTAAGTAAAATATATCCCAACTCAGCAGCCA
TTCCAGTGGGGCAGCTTTTCAGGTGCTATTAGAGCACCAGATACTAGAAAGGAGTTTTTAGGAATATTTTCCTTCTTCCCAGATACTGACAAAG
TGGTCACCAGGTAAAAGTGTGTGATTAAGTATGTGGTTTGCAATCGAGTATAGGCTCCTCATGTAGAATATAATGTTTATAGCTGGGGCAAGGG
GGAACTTTCATTGTTGGTGTTTCTAACCAAAGCCTAGTCATCCCAAGCAAAGCGGAGCAGTGACTGGAATGTATTACAGGGTGATGCTTTCTAC
TGCATTTCATAATGGCCAGCTCTGGAGTCCTTTTCTTTCTCTTTTTCCTTCTCCTTCTCTTGCCCCTGTTCCTCCTCCTCTTGATTCTTTCATT
TCATGCCAATTATCCTCTCCCTTCATTTGATCTGAGAATGGAAGCATTCTTAGCAGCCTCAAGTGATGAGGAAAAATGTTAAACTAAGACCCTG
GCTGCTACCGATACTGTCCAAATGCTGAATTACATGTCGGTCAGACAGCTCAGCTATTTGAATTTCCCCAACACGTGGCAGTTCTTGTCCTGGA
GCCAAATGCCCACACAGCAAGCATACCCGTGCGTTACACACACACATACACCCCCGGTGCTGGGCCAGATGGCATGCTGTATCTCTAATCAAAG
GCGCACTGCAGCTTTGATTGGCACTGGCCGTCCAGAGAGGAATGTGCATGTGAATTTGAGGCAAGGTGAGCCGAGAGGACCTGAGTGCCAGTGG
ATCTGAGACTTGCCCTGAATGAGGGACTTTATCCTGAGGCAAGTCTGTCAGATCCCAGCTCCTCAGACTCAGAAGGAGAGTGGGCAAAGGTTGT
GAGACATGAATATTTGATTAAGGAAAGGGCTAGAATAGAGCCGTGTGCTCACCCTTCTGTGTGTGAGTCGAGTTTTTCCCCTTTTCCTGGACTT
TGAACTCTACAGGGTTTGTGGTTAACGCTCATGAACCATGGGTAAATGAAAAGGTTGCCAGGCCAGGAGCCAGAGGATGCTGGCAGGGGTGCTG
TTAATAGGGCCTGGGGGTTTAGTCCACGCCTTATTCACTGGAGTGAGGCTCCTCAGGCTGTGGCCCTAGGTGATTGGCACCCTCGCTGGTTCCC
CTCCCATCAGGCTGCTGCCCTGAGCGGGTCCCAGGCTGCTGCCCTCAGCCTGGCCCCCAGCCGTTCCTCTGCATTAGCTGTCCTGATTGCCCTT
CTGCCGGCTGCCCAGCTGCACCATCATGTCCCCAGGTGCCGTCAGAGCCCCTTTCAAAAGGCCAACCTTGTTCTTTTAGCCTTTGTACTGCGTT
TCCTTACTTAATTTCCAAATCGAGGGTCCCGTCCTTCCTTCTTCAAACATTTTTCTTCTTTCCTCCCTTTCCCAAACACCTTCAAATCCATGCC
AAACACTAGAAAGAGAGAAGTAAGAAGTAAGTGAACTGGAGAGCTGAACTGGGGGGGGGGGAAATGAAGCTTGGCAAGGCAGGTCCCAGGATGT
CCCAGGAGAATAAAAGACATGCTGTCCTGCAGCAGAGGGGTAGGGAGTGGCAGAGCCATTTATAAAACACCTTCCAAGTGCCAGGCCTGAGAGG
AAACGTGAGGACCAAGCAGGGAAACAATAGGAATAGACTCAGAGGGGTTAAGTAACTTGTACAAAGTCACACAGCTAGGAAAATAGCAGAGCCA
GGATTTGAATCCAGAAATGGCTGACCCTGTTGCATCTACCATTCTAAGCTATGCTGACCACATCTCATCAAGTCAGTGATCAAAAGAACAGAAA
ATAAAGATAGAGAGGATTAGTCATACATCCCTTTTCCCCTTTATTTTATGGGACTGTTTTTAAGGAGCACCTTCTAGTGTGTCTGTATGAGGGA
GTTGGAGAGACCTTCCTTTTCCAGCACTGGGACAACCCAGCTGTTTATAAAATTCCTCCCGAGGGCCTGCCCTGCCCTGGCTGCTTTACGAGGC
AGAAAGGGCCTGGTGTGTGTCTGCTGGTGTACTCTGCAGATGGCATGTGTGCTGTGTGCTGAAGTTTCTGCTGGGAATTAATCAGCTCCTCCAG
ACAGCTGGTCCGCATGCCTAATAATATTTGCAAGTCGGGTTTTATGTTACCCACAGGCCACAGCCATGCTGCATCTTGATTATGGGAATGCAGG
CGGCATTCGGAAACATGCACCAAACACAGATATTTTAGGACCATGAATGACAATTGTTTTCAAAACAGTTGTCAGTTTCCCATCTCAGCGTGCA
CGGAAATATCTGTCACCCCTTGGGCAGATGAGGTGGCATTGCATAGGGCACAAGCAGAATTGGAGTGTGGAGCTGGGTTCCACAGTCCTTCCTG
TGCTCCTTACTTCGCACCATCCCAGCTACCCAGGACTGAGCAGCAGCCCATGTCAGGGAGGTGGTCAAGGCCAGGCCCTGAGCCTGTGAGGTGG
TCTTCCACTGACAGCACTCTAGAAGCAACTAGAAATTTGGGGCGATTGCTTATTCTCTATTCCCTGGACATCTCACTCAATTACAGCAGATAGT
TCGTCTTTAGTTACAAACACTTTGCTAAATTCTGCCCAATCAGAGTCTCTAAATAATGCCAACATCAGTTATTTGAGTGCCTTATCTATGAAAG
GACTTTACTAGGAGCTACTGATATCAAGAAGCCTAAGACTGCACGACAGTCAGCAAGCATGAGGAGGAAGACGTGGGGGATTGTTGAAGTCAGA
TGGAACTGGTTTTGAAATCCAGCGCTACCACCTGATGTTCTGGGGTCTTAGGCAAGTTATTTAACTTTTGCCTACCTCAGTTTACTTATCTGTA
AAATGGAAAGACATTTTGTCTGCTGTATGCATTTTTGCAAAGATTAAGTGAAACAATGCATGTAAAGTGTTTAACAGGGTACCCAGCACATAGT
GATAAATGTTGCTTGTTACTGTTGGTATATCTGGTACAATACTAAGTTCTTGGGGAATAAACAAATGAGTGAATGGATTGTAGATACATGGAAA
GTCTCAAACACTATTCAGGAAAGGGAGAGAAGAATCAAAGATAATTCCCAAGTAGTCAGATGCAGGTGATTGTGAGAATGGCATTACCAATAAC
AGAAATGAGTACTTGGGAAGATCTTACTTTCAGAGAAAGAGGATGCATTTGGGTTTGGATCTGTTGAGCTTGAGGTGGTGGTCTTGGTCCTCCA
AGCCCAGAAAGATGAGTCGGGGTGCCCTCACTGGGTCTCACTGCCACAGCTGGGCCCTCAGCCTTCCAGCCCTCCCCTGAGTTCTGGTCACAGC
CACCTCCCTACCCCACCACTGCACCTTCTTCCCAGAATCATTCCAGAACTCACCACAGGCACCACAAGGATCTGCTGTCCATCTTCTTGTCCAT
ACCACACCTGTCTCCTGTGACCCCCGACATAGGCAATGTCACAAGCCTTGCTTCTGGAAATTCACAGCCCCTCCAGCCCTCACTTCCTTCAGAG
ATTGCACAAGCAGGAGAGAAGCAGTGGGAAAATCCTCTGCAGACTCTAGTTTTTTCTTCATGAGCTTTCAGTGCCGACTAATGTTGATTTAAAC
CTAACAAGTTCAGACAGACATGGCTGTGTGTAAGCATAACTTCCTTTTGTGGATACCTTTTTGTTCTTTATGAGCTATAACACACATACAGTAA
AACGAACAAATCTGAAGTGTGCAGCCCGATGAATTTTTATGTACATATATGCCCCTGAATCCACCCCGAAGATAAAGATACAGAATACCTCCAG
CACTCCAGAAGACCCCTTATTCCCACTCCCAGTCAGTAACCACCCCCACAAATAGCCTTTTTTCCCCTTTTCACCATAGATTGGTAAGTTTTGC
CTGTAAATGAACATCACAGACATGAATCCTATAGTATTACTCTTGTGTCTGGCTTCTTTCCCATTATGTCTGTGAGATTCTTCCATATTGCTGC
ATGGAACAGTAGTTCTTTTCATGGCTGAGTAGTATTCCACTGTACAAATATATCACAGAGCCTCCATTCTACTGTTCCTGAACATTTGGGGTTA
TTTCCAAATTGGGGCCATTATAAATACGGCTGCTGTGGTGATACACACATGCCCTCATCTCTCTGGGCTGTGTTCTCCAGTGGAATTGGTAATT
ACAGGGCAAACCTAGGTTAGCAATTGCAGTTACTGCCTAACAGATTTTGTGGACATATTTTTAAAAAGGTGAATACACACTTAGTGTGTCATTT
GCCAGTACTGTACTTGCCTCTAAGCTGCTAGTGAGGCAGCCTAGTGAGACTGAGGAACCAGGGGTTGGACATTAGGATCCCTGGCTTTGAATCA
GCTCTGTCACTTACAAGCTTGGTGTCCGCAGGCCAGTTACTTAAGCTCTTTGAGCCTCATTTTTATCATCTGTAAAATGGTGATAATAAAAATG
ATCACCTCACATCATTAGTGTGAAACTTACATGGGCTATCTATAAAAGTTCTTTGAGAGTTGTGAAATGTAAAAATCATCCTTATCACCTCAAC
TTTTTGTATTTGACATACATTTTCCTTGAAAACAGTCTAAAAAGTTTTTAAAGAGAATCACTATTAGTAATAAATAGGGAAAAAATCAATCTTT
CAAGAAAAAAAATACTGTCTCCCTATTTTATAGCCTAGGCCAGTCAGTGGATCTGGGCAGTCACCACTTTGATATACCTGCATGCTCCAAGGGC
CCCCTCACCCCGGACACAGCGGGGAGTCACACATCCACTGTCCTCATCTCCGGCCAGCTTGCCTATTTTCTCATATTTGCATTCTCTGTTGAGA
ACTATCCCTGCCTCTGCGCTGCCTCTCTTGTTTTTAATCTTGTGAAGTCTGAATATCAGATGGGCCTTCTCAGCATTTTCACTGAGGCATGTGC
ATACACACATCAACATGAGCACACATGAGCACACACACGCTGCATGAGCACACACACGCTGCGTGGGCACGGCTGGAGCAGTTCTCCCCAGTAT
CTGTGGAGTCGGTCTTACTGCTTCTCTATAGAAGTGATTTAGTTTAAAGACTAGCAGCGGGCCCTTGTAGACACAGAGCACATAGTAGTATTTA
ATAAATATTTGTTTACAGCAATGGAAATAACCAAGGTCACCAACCCCCCCACACTCTTGTGAGACATGCTCTCCTGCTGTCTCTCATCAATGTG
CATGGCTCTCTCTGGGAATCAAATGCTGTTGTGGCTGCAGGAACCTCCAGAAAGGGTTGTTTATTGCATTGATATTCCTTGCACACTCCAAGAT
TGTCTTTGGAGTGATGGGAAGCACAAGAGCTTTGGAGCTAGATAAGCCTGTGTTCACATCCTGACGCCGTATTCCTAGAGCATAAGTTCTGGCC
CCTCTGAGCCTCACCTTTCTCATCATAAAATAGGATTGGTGTTGGTTACCTTTAAGGTGGTTGTAAGAATTCAAGAAGACAATTAGTAGAAGTT
GTTGGTATTATTCACTTCTTCCAGCTCCTCCCCACAACACTTTGGAGTTTTTTTGTTCTGTTTTATTTTGGTTTGTTTTTTGTTTTTTGGGTTT
TTTTTGGATAGCTTGACCTCTTTTCTTCTTCCCACATACACCTCAGTCCCCTCCCAAAGAGGGCAGGGCCTGGACTGGTTAAGAGCATGACCCA
TGAGATCAAAGTGTCTTGGTATAAATCCTAGCATTGCTGCCAATTCACTGTGAGACCTTGAACAAGTTGCTTAACCTGTCTGAGCTTTTATTTC
TTCCTCTTAAAATAGGGATAATAATAGTTATCTATATCATAGAATTGTATGGCTTCCCTGTGAATAACCATAAAGCAGTTGGCACAAAGCCAAC
ACTGAGCTGATAGTGGTAGTTGGCAGTAGTAGTAATAGTAATATTATTATAATATTGATCCAATGGATGCCAATAGCTGTGTTTCTCTCTCTCT
CTCTCTCTGACACACACATACACACACACACACACACACACACCCACCCACCCACCCCACTTTGGGTTTTCCTGATCGCTCCCATCAGGGCTCC
TTCCACATGGAGGCTCTGCCTCTATCATCCTTTCTCCTGATTTGAATCCTCATGAAGGACAGGAACTGGGGTTGCTCTATGGTTTTGGTTGGTT
TGTTTTTTAAGTTCAAAATTAATGAAACAAATGTCAAGGAAACATAATCTTTGGATCTTTGGACTGTAAATAAAAATAGGAACATTCGGGTCTT
TGTATAAGACTGCTGTAAGTTTAGGCTTATGTATTAAATAGAAGAAGAAAAATGTGTCTGTTCAAATGCTCCCAGTATTGAGGGTTTAAATTGA
ATAGATTATTGTCTGGAGGAAAATTCAAGTATAAATTAGACTACGAAGAATTACTAACTGTTGCAGTTTTCATGTTAATTATTTCATGTTAAAA
AATTGTTACCGCATAAATTAACACATTCTTTCATAGAAAACATATTTTAGAAAGATTTGTTACTTTAATAACTCTTTGTGTATTAGAAATCCCA
GATGCCTGTACTAATTAAATATAATCAGATTTTAGATTCAAAAAGCACAGTAGCCATTTAACACAAACAAGCAGGCAGCTAGGTTGACAAACAC
ACTCAGGAAGAAGTGGATCCCTGTTTCTTTCGAATTTTGAATTTCACAATCCAGAACTGTGAATTCAATTCCTGTCCTTCCTTTGTATTACAAC
GACTGGGGATTATTAGACTCAGAGCGCCAGCTTTCCAGCTCAAGGGCTCCTGCAGAATTATTAATCAGCCGCTTCTGAGGCCCCTGTAATCCGG
TGGCAGATCAAGGCTGATCTCGCCATGAGATGCCCTCCCTCTGCCCCCGTCAGCACATGAATTTCTTGTTCTCTGTCAACAACAGCATTGCCAT
TCCCGCACAGTGTGCTGTGACCTGAACAGCAAATATCAGGAGAGAGAAAAGGAAGAAAAAAAAAACCTTTTTAATTACTGAATTTAATCTAATT
CTTCCCATGCTAGAGAAGCAAACCATCCTTTCATGAAAAGCAAACCCTTTAAAATGCAAGCCATTGTCTTAAGCACCGACTAGTGTGTTCCCAA
CTCAGTGGTGCCGCCATAAAATGACCATGCCAACCCCCACCTCCCCAGCCCACATTGAGAGTCTATCATCCTGAGGGCTGTCTAGGGAGAGACC
CCAGGGGACTTGCCCGACAGAATGCCCTGCCCTCCGAGGTCAGACACGGCTTTCCCGCTGAGCTGAGTCGCATGCCTGAACCGTGCTGCTTGTA
ATCAAGCTTCCCGGATCCGAGTTTGGTACACTTCAGTCACTAAGCAGAAAAGCTCCTTCATTTGTATTCTGTCGCTTCAAGCTCTGAGCTTCCA
CCTCGTTTCTGGGAGGGACGGCTTTGTTTATCTGCTGAGCCACCGTGGAGCTTCGGGGCCCAGATTAATAGTTAGGGGGAGCAGGTCAAATTGG
CAGGGAAATGGAGATGAATTGTAGAGCGCATCCATTTTCCTCCCGGAAAGGGTGTGTTGAAAGCCAAAGATTAAAATTAAAAGAAAAGGGACTC
TAAATCACCCTTTCCTACTACAGTCTCTCTTTAAAGATTAAAACTGTTTCAGGTCGGAGATGGGACCAATTTCTTTCATTCCCACCCTCGGGCA
GCTCTGACTCTGCCCAGAGGATACAAATGGATTGGAGGTAAGCAGTGGTCACCTCAAGCCCAGCTTCTTTGCACACCTTTCCCCTTTATCTGTG
CTGGTCAGTCCCATTTTGTATTCTGTGTGAGCATCTCTTTCTCCTTTGTGCTGTTTTGGGCAGAGCCTTTTGATGGTTGTTTATGAATGGTCAA
GAGCAAGTCCTCAGTCTGTGCAGCCCACGTTTCTTGGTAATCATCAAGAAGGAAGCAGATGGTCACCCTCTGTGATTTTAAAGTGTGTAAATAG
GAACTGTAGACTGCCATCAAACATCAAAAATGTCAGTTGGCTTCCTTGTCTTGCCTTGCTGGCAAAAGGAACTCCTGCATTTGCATCATCTGGG
TAATCATGGACACGCAGTACATTTGTAAACTGCCCCAAGTTCTGAAGCCTCATTTCTTACAGCCAAAGGAGGGGGCTCAGTGGCATTGCTCTGC
CATCCCCAGGGTGGCTCCCCCATTGGCACAGACACTGTACCAGATTGAAAAGGGGAGCGGTTTGAAAAAGCCACCCCAAAGTGGTGTTTGTCTT
CTGTATATCCTATTATCCTTTGATATGTTCTGAGCTCCCAGTCAAACCCCATGACCTCCAACTGTTCCCACACAGTATCCCCAAACCCAGTGCT
GCCCATGCAGCCCAGCTCGTCATGTGGCATGAAGTCAGACTGACACGGGCCCTCTGCCCCTTCTGGGGGTGGCTGGATTCTTGGCATCATTTCT
GTGTTGAGCTACATCCCACACATCCGTTGATCACTCAGGTGAGTTCCTCTCACCACCCTGGGCAGCAGTGTGCAGTTGGTTACAAAGCTCTTGT
CTTTTGTTCTGGATTGAAAGTCCGCAAACATAGCGTTTATGCTGTTGAAGCTGTGAATCAGAATTTTTATTCTGTTTGTTTTGTTTTGTTTTGT
TTTCTCTTTACAGAGGTAATCAAGTTACAGAATTTTTATTCTGATATTACTTCCTTTCTGGCTCTTCGGAAACTGGAAGGATAATTGGAATTAT
TCAGAGCCTCAGTATTCTAGAACAGCAAGCAGGGCTCCTTTTTTCTTCAGTCCCACCCAAGATAACCACTTAAATAAGCCTCCATAACACAGTC
TGTACTCCAAGGAAAGTCCCTTCGATGGTTCTCAGATTTCCTTCTTAAAAGGACTAAATACCCACAGTTTCAGGAGATGCCCATTAATGTTATC
CTCTACTTTTGTGTTCCCCCCATAAGTTACCCTGGGTTTGTCATTGGTCACCGTTGCAAGGAGGGTGATGGAGAAAGGCAAGCAGCCCAGAGTT
TCCTCCCTTCACTTCCTGGCTCCTCTAGGGATATTTGGAAGGAATGTCACAGTTTACCTTCTGGTAGCCTCCATGTCTGGATCGAATGCCCTTC
CACAATTTATCCTGCCTATCCTTCCCCGACTGCAAAACCCCAGGATTAATACCTAGAGTCCAGCCTTGCCCCTGCATTCTCCCTGGTAGCTGAG
CCTACTCCCCTGTGTCCTCTAGTTGTCCTGGGTTCAATTTTTAATACCCACTGATGGACCTTCTGTCTCTTCTCTGTCCAGTCTTGAACTCTGG
GCTCTCTTGGAACTTTCCTCACCCCTCTCAGCCTGAATATTCCTTCCATGGATTCCACTCAACCAGACTTTGGATCTGTGCCTACTTAATCAAC
CTTATCTTTGCAATATGTTCGGGCCCACCTTCCACTCCTTGGTTCTTGTCCTCCTTGGCCTAACTTGTCCCTTCTCCACTCACATCCCCGGTGG
GACAGCATTCCTCCTTCCTCCCAACCTCCCTCCTTCTCAAACTTCTTGCTCAACATTATGAAATTTCAGATGGAATATAATCTTAACCCATTGT
AACATATACTTTTCAAAAAAACATGATGCTAAAACTTTATGAACTTGAAGTAGAAGTTTCAACAGAGTGCGTCACCGGGGATAACTGGAAAACA
CCCAGTTGTATTACTTCAGAAGCATTTGGGAAATCTGAAAGTCATTCCTTTGGCCTAGTGTGAAAATTCTGAGTTTTGCTTTGCCAAGTTTCAT
TGGGTTTGAAATCATTGTTTTGTTTAAATAACCACAAAGTTCCAGGTCAGGTCAGCTTTGATTTTGCTTCTTTCACAGGATATCCTGTCCTGGT
ACCGCCTTGTCACCTCCCCCTCCTGTAAACCAAATTTCTGTTATTGGCATTGTTTGTGATTTTTTTTTTTTGTTTTATTACCCAGAGTAACTTT
CAAGTTGAATTATTGCATACTATTAAATATTGGATATAATAATTTGCAGAGTTAGTGGCTTTATAATTAAAAAGGGCTCTGGGTGGCTCTTTGG
TTCAGAAAAACAGTGTCTATTGTTGAAGTTAATGGGTTTCCTCCTTGAAAATGCTATTAGGGCACAGCTCAGATTCTCTGTAGGAAGGATTTGG
CTTCTTTTATTCAATACTGACTTCACCTTCTCCACCTTGGCCTAGTTTTTTAGCCAGAGGGGATATTCTCTGAGACTATGGAATTGGCCTGGTC
ACCAAGAAAAATAAAAAAGAGAGAGACTTGGCAGTTCAGCTCTCCGGAGATGTGAGCTGGGCCCACCCAGGCCTGTCCTGGCCCCTGCCGTGAC
CCTCCCCACCGCAAGGAGAGCCATGGGTCATGCTGCATCATTCCAATGCGGCTGCCTCTCCCTGAAGCCCTCATCTCCTGCTTATGTTCTCTTT
TCCTGTCTCTGCTGAGCCCTCTTCTTCCTAAAGGCAGTGCCACCATTTAGTTCTCTGTTGGGTTTGCCCGTGAAGTCTGTCTCCCTGGCTGCCT
AGAAACCCCAAGGGCGGAGGCAACCTGTGCTTGGTTTTCCCAGGAGCGCCTCACAGGGTGCTGGGGACATCATGGACCCTCAGAAGATTCTCAG
TGATTGGTTGATGAGTGTTTTGATTAGGAGGGTTTTTTCATCTTCTTTTCCTCTTCTGATGTTTGGAGAGCTTTGGCATTGCTGATGGCATTTG
CCAGTGCAACTCAGGGACTTTAGGCCTTTACTATCTGAATAAAATAACAGCTGGTATTTATTCAGCTATTACCAGGTACCAGACGCTGTGCTGA
GCCTTTTACATCAACTGTTCTTCCCACCACAACCTTAGGAGGTTATCATCCCCAGTTTACAGACAAGGAAACTGAGACTTAGGGATGTTTTAAA
TGATCAACCAGCAAATCCAGTGTGCAAGCACAGGGATACCTGACTCGGAAGCTCCTTTCCTAAATGATCATAGTGGTCCTTCCACAAGTGACCA
CTCAATCCCAGCTCAGCCCACAATCGGACCACTTTGCATTGTCACTGAAGAGCATCCACTCCTCGTTTCCTGGCCGGCTTATGTACTCTTACTG
CAGGAGGTGATTTGGCAGCAAAAGTATCTGCGGTCTTCATTGGAGCATTTGAAATGCCCATGCCATCATCTTAATGTATGAAAACACATAAAGA
ACAGGAAGTAACTGAGTCAAGTAAGCTATTTCTATTTGAGGCAGAAGTGAAAAACAGATTACCTGGCCACTAGGGATAGAGGGACTTCATTAAC
TGGAATGTGGCCTGGGGCTTGGGATGGGCCATCCTGACATGATGACTTTGGGCAGTCATCCTGTTGGCACTGAAAAACCGAGAGCATCAGTCAG
GCTGTGCTGTCGTATGAAAGGAAGATTTTATTCCTATAACTTTTAAGAGTGAAGAAAATTATTAGTTTATTACACCACTGTATTGACACTCTTT
GCGTTTAGTTTGATTAAGTCTGTGTAAAGGAAGAGAATTACTTTGACCGTCAGGGTGTCAGATAACTTACTGCCTGGCTGGACCCTTTAGCTAG
TTAGAGCATTTATTGCTGTTTAACCAGCCTTAGGCAAGTTGCTATGAAGAAATATGTGTCTCCAACCTCAAAGGATTTGCATTCTATTTAAGGA
GATTTTTTAAATAACTTATGAAAGAACTAGTAAAAGGACACAGAGGAGACATGGACACAGTGGAACAGTGTATAGTGTTTATGCAGTCAGGCTC
TGGCACCTTCGGTTCAAATCCCAGTTCTGTCATCTACACTTGTCCTTTGTTTGGCACATTGCTTTCCTCTCGGTGTTCCGTTTTCTCATCTGTA
GAACAAGACTATTGCTGGCATCCACCTCATGTGCCCAGTGCTTTGCATGCCTGACTCTTAGCAAGCGCTCAGTGTATGTTTAGCTATTGTGGTA
GCTGCTATTATCAAGCATTAAACTGTTGTTTCTAGCACTGGCGCTAGGTGCATTGGAGAGATTAGTGAAGGCTGGAGTAATCACAGATGGCTTC
ATGAAGGAGGTGGGACTGGAGCTGAGGAGGCTGTAGCTAAGCAGGTGAAGAGAAAGAGAGCATTTCCAAAGCCAATGTAAAAGAAACAAGTGTG
AGCATGCTAAGTGCAGGACACAGGTTATAGTAAAGCAAACTGAACTAGATTAACTGTGTTTGGGGTAGCAAGAACCAAGTTAGGTCAGGCACGG
TGGCTCACATCTGTAGTCCCAGCACTTTGGGAGACCAAGGCAGGAGGACCACTTGAGCCCAGGAATTCGAGACTAGCCTGGGCAACATAGCAAG
ATCCCATCTCTACAAAACATAAAATTAAACTAGGTGCAGTGGCTCATGCCTGTAATTGCAGCACTTTGGGAGGCCGAGGTGGGTGGATCACCTG
AGGTCAGGAGTTCAAGAGCAGCCTGACCAATATGGTGAAACACCGCCTCTACTAAAAATACAAAAATTAGCCAGGCATGGTGGCGTGCACCAAT
AGTCCCAGCTACTCAGGAGGCTGAGACAGGAGAATTGCTTGTACCCATGAGGTGGAGGTTGCAGTAAGTCAAGATTGCACCACTGCACTCCAGC
CTGGGCAACAGAGTGAGACTGTCTCAAAAAAAAAAAAAAATTAAATTAAATTAAACATTAGCCAGGCATGGTGGTGCACATCTAGCATCCTAGC
TACTCAGGAGAGGCAGGAAACTTGCTTGAGTAAGCCCAAGCAAGAAGAAGAGCCCACAGGGGAGACACACAAAATGCTTGGAAAGAAAGGAAGG
ACATGGGGAAGAATAAACTCAGAAAAATAATTCATGCATTTCTCATAGAAATGTTTGAGCTTTGCGGGTTGGAATATGCTAAGTTTTACTGCTT
TGTTACCGAAATTATTGGTATTATTCACATGTGAGAAGTGTGCTAAAATAGTACAAATGTACCCAGAGCCACTCGTTAAGTAAACATGACAGAA
GTGACAATGGAAGCATCATCTACCCAATTGCTAATTGCTGATAGACACCCATGGGCCTTGAAATGTCACGATGCTTTTGAAGAGTTTTGAAAAG
TCAAAACACTTTTCCCATTCTGTCTCATCATAAGTCATTCTCAAGGTAATGATGTGTTATCTTCCATGTTTTAAAAGACAAGGGGAAAGAACGA
CGTGGGAAGTCATCGCACTTCTGCTCACAGCCCAGTGACTTTCTGTTGCGCTTAGGATGGAATCCAAAATCCTAGTACAGGTTACAGAGTCCCA
CAAAATCTGTCCTTGTACCCCATCTTATTATTCTTCATAGCACTTTATTGTAGTTGACATATATAATAGCCATTCATTTATTTTATCTCCCTCA
AACTAAGCTTCCAGCTCCATGACTGCCAGAGACTGTGTTCCCTTTCGTCCTCCTGGCACCGAGTGCAGTGCCAGGCACTGGGGAATACTCCAGA
AATACTTGTGGTAGGAAGAAAGAACAAGCCAGCCCAGCAGTGGTGGGTGTTTGTGTTGTTATTTCTGTGGACAAACTTTGATAACTGAATAAAT
CCACAGCCCTCTCCTTTCTCTTGCTTGAAGCATGGATGATTATTAACTTTATTTTAAATGAGGAAGACCAGGGCTTGGTGAGGTCAAAATGGTT
GATGTCTAGTCACTTGCATTCATCCCAGTCTATTAAGAATTTATTCTTAATCCAGTCTATTAAGGATTTTCCACTTTGTTTCACTGTGAACTCT
CCCAAGGCCCCTTGCCACCTCGTCTTTCTGCTGTTGTAACTAACCACCCCCATCCCCAGCTGCAGCCCCAGTGAACTGGGCCCCCTTGTCCTCA
GAGAACAAAGTATGGGGCTCCCCCACTCAGATACATTCACTCCGACCCAAAGGGGATCTGAACACACTTCCTTTGGCAGTGAAAATGCAGGGTT
CTCCAGGTGTTTTAAGTTTCATTAGTGTGTGGCTGTTGCTTTTCTATTTCAATTGGCAATTCTTAGACTCTTAAAAGGACATCCAGACAGTGAC
ACTGGTGCTGTCACCAAGGACAAGTAGAAGACTCACCAGAGGGCAGGGAACTGATAATACGCTCTGTAAGGAACTGTGGTTATTCAGCCAAAAA
GGGAAGGGCTGGGAGGGCTGACCAGCTACTCATTCATTCATTCAGCACCTGTTTATCAAGTGCTTTCTCTGCAGCAGACACTGTACTCAGTACT
GGAGATGTGGCCATCTGGGAAGACATGCTCCAGCACTCAAGCACAGTATTTATCTGGGGCAGAGACAGCCTTCAGACAATTCATGCATTCATTG
CCCTTTAATAATAACAGCTTCGTTAGGCAATATTTACTGAAAAAAAGTAGTGACAACTACAAAGAGGGAGGTGCTCTGGCAGCATAGAAGTAGG
AAACCTACCGCAGTCTTTAGTGATCAGGCAAAGGTTCCCTGTGCAAGTAACACTTTGGCTAGGAGCAAAAGAGAAGTAAGAATTAGCCATGGGT
TAAGGGCTTTTGATAAAGGAAATAGCCTGTCCGTGAAGCAGGTAGGAGCTTGGCATTTGCAAAGCAGTCAAAGAAGGGCAATGGAGAGGTTGCA
GAGGAGAGAGGAGGGAAGCAGAGGAGAACAAGTCTGGGATGATAAGAAGCAGCTGGGGAAGTAGGTAGGGACCAGATTATGCATGTAGATTCTA
TTAGAAGGCTACTATAGAAACCCAGGGGGGGACATGCTGAGCAAGTGAGGACTAGAGGAAGGGTAGTGAATTCAAGATCATGAAATGGTCAGGA
TGTAATGGACCTGACACGGGCAGGGACAAAGCAGGAGAGCACAGAGATCATTCAGTAAAGATGTATTGACCTCCTTCAATAGGCAGGCCCTGTG
CTGGGATCTGAGTGCACAGTGATGAAAAATAGAGTGTCCAGGTGAGGATGATTCTCAGGATTCTGTTGTCACCAACTGGGTGGTTTACAAAGTC
ACCAAGGATTGAGAAGCCAAATTGTGGGGAAGATAAATTTTGGATGTTGCATTTGAGGGGCACTGACTTGTATAACCAATTTCATGGAGAGACA
ATTGAGAGGGAAATTGGTTATGCAAGTCAGGGACTCGAGAGAGAGGTCCCATCTGGTGATATAAATGCAGGCATCATTGGGGTCAAGATAGTCA
TTGGAGCCATGGGATTGAATAAGATCCCTGATATTACACAGAGTTTGTGACCACTTATTTCATATCTCAACAGAATTCAGAGAAACAGGATTGA
GCTCTATATCAGAAATTATTTAAAACATCGACACATTTATTCATTACAGGAATAATTCAGCCTAAGGCTAACTGGTCTTATATATGTTCCTGGG
AGTTAAATGACTAAGGCACAAAGATCCTCTTACATGGAAGTTCTAGGACATGGGTACCAGTGACAACACCAGGTTGTCAAGTTTTCACAATGAC
AAGTCGAGGTTTCGGCAATGAGTTTACCTTGGGAGTTTGGTAAAATTTGCAAATAGTACCTGGAAAACATTCAGTGGAAAACATGCTTCCAAGT
ACACACACCACACACACACACACACACACACACACACACACACACACACATATTATGGCCTTCGTGTATTGGCTTATCCTAAAGAATCCTAAAC
TGTTAAGCTAATTCAGTAAGAAATGACAGAGTGGTCACTTCTTTTTTTGAATTCATGTAAACATGGAACAAGTACAGTAAGTGTTACAGTGAAG
CTCAAGATATCAGTGACATAGATGATCATTACCTGCATAGTTTGCTCTGTTTCCTCTAAATAAAATGCAAGGAGGAGAAAACTGTTTTCATATC
TAAATCCTGCTCCCAAGCAGAAGAATTTCAAAGTGTGAATCAGCTAGTTACATAACAATACCAAGTTAACTGCATTGCGGTTCTCCTGATGTTT
TCTCCGTTGTTCTGTAAGTCCATACTACTTAGAAGACCACTTTGATTCTGACTTTTTGCCTGTCAGAACTTTACTTTCAAGGGAAAGGCAACGT
TTACACCCAGGAGATCCTTCCTTCAAGGTGTCTGAAGTCTCCTAACTTACAGAGCTCACTGCAGCAGCCCTGCAGCAGGGACATCTGTGGGATT
GTATAGGTTATGTACTACACAACCTTAGGGATGCTGTTTTCATAGACTGGGATTTTACACTGTGCAACCTGCAAGGCTAGTTTTAAAGACACCT
GCCAAAAATCCCCAGTTAACTCCACCACATTTAGAGCTGCATTTTAGGGCCAATAGCTTTGTACCCTCAGGAGGTGGGAAGCCAAGATAAGGGG
AAACAGAAGTAATGAGATGTGTCCAAGAGGAAGCAGGGATTAGCTCTTTTCAGTAGAAACCTCAGTTAATCATTAATACCTTATTGCTGCTTTG
ATTTAACCTTTTGTCTTCCTTGAAAAGGAAAATGAGTTATCTATAAAGAAGCTTCTTTTGTTGAAAGCAGCTGCTGTTTTAAGACAGAGAAGAA
GCAGGAGAAGTTGGACAAAGACATTATGTTTTATTCCTCCAAATACATTATAAATACATTGTTTGGTGTAAATATATTGCTTGCCTTAGAAAGA
AAATTAATACAAAACAAATTTAAGTAAGTTCCGACTAGTAGTAACTCAGACCACATATCTACAAATGACTTCCTTTCAAAGATTCCTTACATTA
ATGCAGCCTTTGTTTTAACATTTTTTGAAGGTTTATATATTATAATCCTTATTGTAGATTATAAAATGAGAAAATAATAAAGCTGACTAATGCA
ACTGGCTGATATTGCCGTTGGTTTTTTTTAATCTGAAATTATGAAATCCCTTGGAAAATCTAACATGACAGTATCCAAATTTTAAATAGTCACT
TTCAGCAACACACACACCTTCACCAATGTTGAGTGATTCAAAGAAGGGGAAAAACAACTCCTTTGGTTGTCATTTTATTTACAGGATTTGTCTG
CACAGCAGAGAAAAATGTTCCATTTACTTCAGAGATGGTCAGAGATTTCCAAGCCATTTGGCTCAAGAGATGGTTTAAATAGGAATGAATGTAG
TATACCTTTAATTCTCTGAAGTAAGAGAACTCCAGCTGAGGTTTGGTCCAGAGCAAAGTCTGCTCTCAACATTGAAGAAAATCTATCACATTAA
TCCCAGTGGCTCAGAGTCCAAAGCTGCGTGAACAGAATGCTAAAGGATAGGAAGTGTGAGAAGAATGCTTTGAAAGAAACCTTAAAAACAAAAC
AACTGACTATTCAGAGAATACCAGGTCTTATGTGGAATCATCTAGCACGTAAGGAAAGATATAAATAATTATGCCTGAGAAACATAACTAATTA
AAAATAACTCCATGCATAAAAGGAAAACTATAGAATAACTTTTCTGCTCCAGTGTTAATGTGAGATGCCTCATAATGAAATAGTAGGATTTTTT
TCTTTCATTTCATTTCACACAGACACATGAGAGATGGAGAGGAAAATTATATTGCCTGAAAATGCCAGGGTAGATTTACTAAAGATGTTATCAT
ATGCTGTATTTAAGACAAAAATCTAATTGCTGTAATTTATCATACCTCTGGCTATTAAAACTGTGCCAATTTAAATATTTTAATCATTCGCCAC
TTTTGTACATAGTGACTTTTTATAGAAGATTCTATGAAATGTTCTGATTTTGTCAGCCTTTGTATGATTTTCAGGAGCTCCTTAAGCAGAATGA
ACATGCTTTAAGCATGTAACTGTGTTTCCATAAGTAATAAAAATTCCTTGTGTTTTTCTTTTAGTTTGTTCAGTAAAATACCTGGAACTGCTTT
ATAATTGCTGAGCTGAATGTTTGAGAGCACAAGCTGCTGAATCCCTTTTATTTTATTTTATTTTATTTATTTATTTATTTATTTATTTTTATTT
TATTTCATTTTTTTCTGAGACGGACTCTTGCTCTGTCACCTAGGCTGGAGTATAATGGCACGATCTCGGCTCACTGCAACCTCTGCCTCCTGGG
TTCAAGTCATTCTCCCGCCTCAGCCTCCCAAGTAGCTGGGACTACAGGCTCGTGCCACCATGCCTGGCTAATTTTTGTATTTTTAGTAGAGACG
AGGTTTCACCCTGTTGGCCAGGCTGGTCTTGAACTCCTGACCTCAAATGACCGCCCGCCTCTGCCTTGTAAGGTGCTGGGATTACAGGTGTGAG
CCACCACACCCGGCCTGAATCCCTTTTAATTCCCTGCACACACATCAAGCTTGTAGAAAGCAAGGTGGACCACACCCTTCCTTTTGGTTTAGAG
ACAGGATTCTTTCAATGATCAAATAAATTTTTGAAAAAAAGGACTCCACTGACTTTGACATGAATTTGCACCTTGGTCTTGAAAAGTCAGAGAC
TCATAAGACAGTCATGGAGGCAGAACTGCTCTTTAAAAAACAAACATTAAATAGTACTCTTGCTGGGTGGTAGAAGAAAAAGTGGTAAGAAAGT
GATCTTACAGTATGGAAGAAAAAACTATCCCAGGAGTAAAGATGGCTACTACAGGGAGAAAGAATGGAGGAAAAACAGCAAATGTGGAGAGAGA
AGGAGCTGGAGGCAGATTCCTTTCCCTGCTGTGGGTGTGGGAGGCCTTTGGGAAATCTAACACATTTCAGCAGAACACATGGGCAGTATCCAGT
TGTCCCCACCCTGGGAGCAGCTCAGTGGTTTAATCAGGTTTTGTGTACCCAGTGAGACCACACTATCTTATATACCCGAGGACTCATGAGAGCA
GCATTATGACTAAATCCTATGTATGTTTATTAAAACATAGCATTATTTCAAAGTTAAATATATATATAATTTTCCTAAATCATAAGAAACCAGG
GCTTCTTCCCCCCTTTATTGAGGTACTAAGGTGCTTTGAGCTGGATTACTTACGGTCACACAGTCAAGACCAGACCCTACATCTTCCCAATCCC
AGCCCATTCCTGCTTCACCTCCTTTCTGTCTTCCCCTCTAAACCTGCTCTCAGGAAAGTAGCTTGCCAAGTGCCTTTCCCTCCCACACCCCATG
CCCATTGCAGTGCCCTCTGCCTGATGAATAACCAAGTGTTCATTCTGCAGGTGCAAAGTTCCCCATCAAGTGGACGGCCCCCGAGGCAGCCCTG
TACGGGAGGTTCACAATCAAGTCTGACGTGTGGTCTTTTGGAATCTTACTCACAGAGCTGGTCACCAAAGGAAGAGTGCCATACCCAGGTAAGA
GCAGGGCCTCACAGACATGGGCCTGAGATTGGAGGCTTTTTTGACACCATTTGGGAAAACTTCATCCACTTTGCTTTCTAAGCCCCATCCTCAG
CTTTCTGCTGAGCTTGAGTTTGAAGAACATGGCCAGGTATTCAGCGAGAGAAAAGCAGAACTGGGAGCCACCATGTATGTAGACAAGCAGCCAC
CATGAACTGTGTGCCAGGATTAATGTAAGTAGCAGCTGGTCTAAATGCTTCAGTCCCCCTCCAGTTCATAGAATCTCTCCTCTCTCAGTAGGAG
TATGTGTCTGTGGTTCCCAGAGCAGAAATGTCACTGGCAGTGCCTCTGCCCAAATGGAGTTTGCAGACATGTCTGGGAAAGAGAAGGAGAAAAG
CCCCACTGCTTTTTGATTACACTGTAGCACTGACCCATGGAGTGAGCCTGGCAGGAAGGTGCCAGGACCTGTTCTCTCCACAGGACCCAGCTCC
AGATGGCCAAAGAGAGCAGAGGCACATGTGAAATGCAATTTAGAGCCCACTCACTGCCTGTTGCTGAACAGTCAAAACATGCTATACCAAAGCA
AGTATAATGTAAAGAAATTGGAAATTCTTAGACTCTGGAATGTTTCTGTATTATCAGATTTATGCTCTCCAAAGAAACCACGAGTTTGGCTTAT
CCATGCTTAAATTGATTAACGTCGACAGATGGTGACATAAAGGACACCCATGCCATTTCACCTCCACGCTTTAGTGGTTATAGCACAGGGACGA
TCTGCTTTTTTGAAATGTTAAGGACATATTTTTCAGTTACTGCTGAGTGAAGAAAGAACATAGCTCCCCCTCATTCCCTGCGTAAGAAAGGAGC
TTAAAGAAAAAAAAAAAAAAAAAAACAATGATAGAAACGATAGTGACGATTAATGAGGAATTTAGGTCTCTTGCAAATCCTGGGTTTGTAATCA
TAGCTTTGCAGTCAGAGGAGCTAGCGAAGGCAGATTAATTTGTAATCCCTTTGTTCCTTTTTTATGATTGTTTCTGGTCATCTTCAGAGAACAC
AGAGAGGGGCACATCAAGATAATCTCGTTCTGGGTTGGTGTAGCCTTGAGACTCCTAGGAGAGCCAAGTTGACATGGAGGCAAAATTGACAAAT
TCCCAGTCTCTGAAGTGCCGGCACTGGGCGTTTAGAGATGCGGGTGTAAGTCAGTTTTATTGATGCTGCGGTGACTGCTGCTCCCGGCATGCAC
ACACGGGCAGACACTCCGGGAATCGCCAACCAGCACAGAAGAAAGGGCCGCCCTGGGGGATGGAGCACAGCGTGGGTGTTTCTGAATTGCTTCT
CAAGGGGGGAAATGTGCGGGTCTCAGGAGGGCGAGGATGTGAAGGCATCTCTTCTTTATTTATTGTCTGAATTTTGAACTGATTTCTTCCTGAT
CCCTCCCAGGTTTATAGTCACCCTGTGTGTTAAATGCAACTGGGGAGAAAAGGTTAATGTTGTTTTGCGTTTCTGTGAAGCCTTCCCTTCCAGT
AATACAAAGCAGCTTTTTGTTTCTCAGGGTCTCACACAGAAACCAAGACAAAGGGGTGGAGGTGGGGGCCTTGGAGAAGGAAGGCAGGTGCGTG
TTTCTGAGGGAGTCTTTTGTCAGGTTTGGTTTTAGCATAAAGAAAATGCCCTCATTTTACCACTTAGACCAGGATTCATTCATTCAGCGTAGCT
TCAGTCACTGTGAACTGTGCCAGGCATTTTCCTCTATCGGGGAGATAGTGCAAATAAGACCCTCATACTCTAAACAAGCCTTGATTAGTACCTG
CCTCAGGCTAGGCATTGTTAGCACAGTGGGCAAGTAAATAAATTTAGTCCCTATGTTCATGGATATTACAGTCAGATAGATAAAATATGCATCA
AATCCAGGAAGAAACCAGCCTTCTTTATTCCTTGTTTATTATCAGAGGGAGTCATCCTGAAAGGATGGACTACTTGGCTTTACCTACTATGGGG
CCTAGATCTTCATTCTGATAATGGGGTCAGGGACACCTGATCCAGGTATCCACACACAGCAGGTCCTGAGAGGGACTCTTCAAGCTGAGTCCTG
TGAGGATGGCAGAGGAGTTTGAAGTGAGAACTAACTCCAGCGGCTCCTGCAGAGAGAGCTGTGAGCAAGTGCTGGTCTGCAGTGGGCTGTGATA
ACTGCCTCTAGCTGTGGAGGAGTGCTGGAGAGGGGGCTGTGTAGGGAAGGCAAGTAAGCCAGACCTTGGAAGCCAAGTAGGTTTCCTCAAGCCC
AGAAGGCGGGAAGAGCATCCCAAGGTCTGGCACGTGGGAAGGCACATAGGCCTGGGAGGACAGTTCCAGGGACCACAGGATGAATCTGAGAAGA
TGCCATGTGCAGGGGCACACCTGGAGATGTGACTGGAAGGGTGGCCGAGAGCCTGCGTTGGGGCCTTGCTGGTTGGCAGTGGCAACCAGGGAAC
CGTGTCCTCATTGGGGGTGTCGTGAGCTGGTCCTCATTCAAAGAAAGCCACAAAAAATTGTTCCCAGGCCTGTTCCTAGCCATGGGTTCTTCCT
GGGTTTTGCTTTTTAACATAGCTATAATCCTATTTAATTGGGTTTGTGAATCCAACAGAAAACTCTTCTTGAGGGAGAAGCTTTTACAGAGGGA
TTCCAATTATAGGATTATTACCCTAACGAAGGATAATAATTCCTAAGGAACAAGAGGTTTCACGAAGCTTCCTTCAGATGTCACTGTGAAGGCA
AAGAAGGACCTTCCTCTCTGCATTCTTGCTATTTGTTGGTGTGGCAAGTCAGTCAGTGTGCGCTGAGGGGTCCTTCTCAGCTTCCCACGTTGCG
TCTTTGTGCCTGTTGCTGGTACCTCAGGACAGACTCCCAGTCCCGGTCCATCATGATTTCCATTCTGCATTTTGATGTAGTCATGTCTCATGTC
TCAGTTGGCTAGAGTATATTTTGAAGTAATTCTTTTTTGGAGAACAGTAGATGGTGGTACATTTTTCTGAGTCTTCCATACCTGAGAATAGCCT
GCTGTTTCCTTGACACATAAGAAAGCTCCTTTGCCTGGTAGGTGAAGATGGAGGTGCGGGAGGAGGGGGTGACATATAAATTGGTTTTATAAAA
GCTTTCCAGGCATCTTTGCCCAGCGTGAAAGAAGGTCCAGAAGTGACAATCCCAGAAGGAACTTAGAGTGGAGTATGTGAGGTGTGACCAAATA
AAGGGCAAAGGGAGGCGGAGATGTTAGAACATTGGCAAGTTGTGTGAGAGGAGTGGACAAAAGAAGGGAAGACGACAATCTCAAACTGAATGAT
AACCTGGAAGCCGAAGGAAAAAATACTGAAATATGATGCTGAGATTAACGGGGGTGGTAGAGAAAAGTTACATCACCTTACCAGATGAGGAAGG
TTGCTTTCGAGATGTGTAACAAGTATGGATGCAGTTCCAAAGAAGAGCTCCATCAGTTCCCCGCTAGCAGCCTGAGCTGAACGCATTCTCTGCA
TTGCAGTCTAGCTTGCTTTTCTGCAAGCAAGAGTTCAGAAGCTTTCTTTACTTCAAGATTAGAACTGATTTCATACTGATGGATTGTTGTATGT
GAGAAATAAAAACACTTAAGTAAAAGCAGTTTTCTGGGGTTGATGCATAAAATCAGTTATGCATTTTTACCTGTCATTTCACCTTAATACCTTT
CTCAAATCCTTCTTTTCCCCTCTGCTTCCAGTTTCTCCCCGCCTCCCCACCACCCACCCACCCACCATTCTATCATTGTTGCCAACAGAGTTAG
TATTCTAGAAAACACAGACCTGGTTATCTCACCCTGCTAGTTAAAAACGTCCACGGCTTCCCTTTGCCTTTCAGATAAATCCTAGAAAACGTAA
TTCTACATAGTTCTTCATAACTCTTTTTCTGTAGTCATTCCACAAATATTTATTGTGCACCTACTATATGCCAGGTACTGTTGCAAGCACCAAG
AATACAGCAGTGAAGCAATAGACAAATGCTGCTGCCCTCATAGACTTCCATTCTTCTTTGAGGAGACAAACAATAAACAAAGATAAATAAGCAA
AATATATTGAATGTTTGCTGCTGGTAACAGCTAGGGAGAAAAATAAAACAGGGAAGGGGAGAGAGGGGAGAGTGGCAAAGGAGCACCTCACTGG
TGAGTAGAAGACCTAAAGGAAGCAAGAATCCAGCCAGGTGGGTATCTGGGAGCACAGCCTTCCATCAGGGGAAGGGAGAGTGCAGAGACCCTCC
AGAATCAAAGGAGGCCAGCATGGCTGGAGCAGAGGGAGCAGGAGGTCCCAGAGGCCCCAGGGGCAAATCAGATAAGGCCTTTCTAGCTCATGGT
TGAAACTTTTGCTTTTAGTCTTAGTGAGGTGGGAAGTGAGGGGATTTGAGCAGGAGAGTGACATGATTTCTCTCTTGCTGTCTTCCATCCTTCA
CACGTTACCAAGACACATCAAGAGATGTCTCTTGAACATGCAGGTCTCTCTTTGTGGCTCCCCTGTCTGCCTCAGAACCCTTCAAAGCCCAACT
GAACATCATCTGCTTTGGAAAGCCTTCCCCATGTCCAGACCAAATGGGGTGCTCCTTCTGCTTGCCTCCATGACACCTAGCATATTTCTGTTGG
GCCCCCCGTTTGTGTGTATTTCTCAGCAGCTTCAGAGAAGGGAGTCCCTTTGATGGATTCCCGAGGTCCAGCACAGGGTCCACCCCATAGTAGA
CTGTCAGTAAGTATAAGCAAATAAATGAGTGAATGAAACAGAAAAAAAAGAACTCACCTATGAGGTTTTTGAAACAAGGTTTTTAAATTTAGCC
CATAGCCTGCAAAGCCTTTTAACTGCATTCCATCATCTTCACACATGGATATATATTTTCCTAGCCACAAGGTAGCCTCTGTCCTCAAAGTTCT
TCTGGAAAATTGACCCTGTGATCATTTCAAACATCATGTTCCAACACTATTCTAGGCAGAGTGTCCTGGCTATTCTGTGCCCTGGCTCTTAACC
GAACACCTGTTTTGTGGATTTGGCAGGACCTGGTAGAACAGGAACACTTTCTTTTTTTTTTTTTTTTTTTTTTTTTTGAGATGGATGGAATCTT
GCTCTGTCAGCCAGACGGGAATGCAGTGGCATGATCTTGGTTCACTGCAACCTCTGCCTCCTGGGTTCAAGCGATTCTCCTGCCTCAGCCTCCC
AAGAAGCTGGGATTACAGGCACCCGCCACCACGCCCGGCTAATTTTTGCATTTTTAGTAGAGCCAGGGTTTCGCCATGTTGGCCAGGCTGGTCT
CAAACTCCTGACCTCAGGTGATCCACTCTCCTTGGCCTCCCAAAGTGCTGGGATTACAGGTGTGAGCCACTGCACCAGGCCAGGAACACTTTCT
TAAGCAAACCACACTTATTCATTCATTGGAAGCACTTTCAAATCATTCAACAGGTGACTTTCTTGAATACATCTAGTCGCTTGATAATGACCTG
AAGAAATAAGGGAAATTAATGAAACTGGTGGCTTTTTCCTTAAAGGATTCCAAATGCCACTCTGGTCCTCCCTCTGCCTTAGAGCCCTGAACCG
TAGATACATTTTCCTTTGTGTTCAACATAACAACAGGAGCAGGTTTTTCATGGGAGAAGAAAGGGTTAGGACTCTCCAACCAGGAGGAGACTGG
GAAGCCGAATGAAAGCTCTCATCTTAGCCGAGGGTGCGTGTCTCTCCCTGGCTCGTCCACAGTCCCTGGGGTTTTTCCTGCAGCTCATGATGTC
CCCTCAGAGAGGGAACCCTGCCTGGAATGCCCAGAGGTCATGCTGTCACCTCCTTCTACAAGATTTCTCTTGGCCAGCATGTCTAGTGAGAGGA
GGAGGGAGACCAGGGGAGAATGCGTGTGAAGAGGGTGGCCAAAGAGGGACTTTCGGCCCCCAGTCCTGTGTTCCAGCCTCACGTGCTCCTTGTT
CTCAGGGGTGCAGTTCCGGGGCCTTCCTCATGGGCACCCTGCCTCTCTTTTTCACTCCTTTTTTTTTTTTTTTCTGGGACAAAGGAGTTCTCTA
TGATACTGTATTATCAACTTCAAATACAGAAATACCTGTGTATTCAGTTACATGAGTAGCTGTCTTTCTGTGTTTATTAATACAATGGGCTGAA
TAAAAACAGCTGTCCTGAGGTTATCTGGCAAGATGAGGAAAGAGAAACAAAGCAAGTCATTATTGTACCTTCTCAAGATAAGTTCTTTATTCTC
ATCCACACTCTTCCATCAGCATCTCCCTGCCCAGAATTTTCACAATGATCCTATACTAAAACCTAACTCAGTGTCACGGTTATTAACCAGTACG
ATAGCAAGGACACATGAGAATCTGTTAATACAAGCACATGCGATGGCGCAGGCTCATGCTGAGCAAGCACCATTAACACCCCTCCTCCTTCCCA
GCGCCAGCACAGCACTGCTCAACAGACATGTAAACACCGGGCCTTTTATGCACAGAAGCAAGGGGAGCCTCAGACCCAAGGCCCTATGCACAAC
GTTGTGTGATTCCCTTTGAACGGAAAGCTCAGTTAGCAAAAGGAATTTGGGTATCTGATAATCTAAACATCATTCCCATTGTTAAAGTGCTTAA
TTATTCACATGGCACAAAAACCCAGGTTTTGCAACCCATAGTCAAATCCAGATGCAAACATGGACGGGTAACACTTAAAATCCCTGATTGTACA
GATATGGCCCATCTGAGATCCATATTGGGAAGCTGCACAAATGGAAAGCTATAAAACACAAACACTCTTCTTTCATAAAAGACATTTTTTCAGA
TAGCTGAAAGCACAATGAATGTTGAGGTATTTTGTTAACAAATGGAGAAGCCAAGTTCAAGGGAAGTCAAGTCACTTGCTAAAAGATGCAAAGT
TAGAAGTCAAGAAGTGACATCTATTTCGGTGCTCTGCCAATGAAAGTGATTTTTATTCCTTCCCTATAGAATCTAAAAGAAGACCAGGTATAGT
GGCTCACAGCTGTAATTCCAGTGCTTTGGGAGGCTGAAGCAGGGAGGATCGCTTGAGGCCAGAAGTTCAAGATCAGCCTGGGCAACATAGCAAG
ATGCTATCTCTACAAAAAAAAAATAAATAAAAATAAAAATCAGCCAGGCATGGTGGTGCATACCTATAGTCCTAGCTGCTTGGGAGACTGAAGT
GTGAGGAGGATCGCTTGAGCCCAGGAGTTCAAGGCTGCAGTGAGCCATGATCATGCCACTGCACTCCAGGCTGGGTGACAGAGCAAGACCTTGT
CCCTTAAAAAATTTTTTTTCAAAGAATCTAAAAGGAAAAAGGGAAGACTACTGAGTTCATGTACAGCTCAAAGAAGTGGAATAGGGAAGTTTAA
AAGAAAAGGGAAAAGAAAACACACACACAACAATAGACACTGTTGGCAACCCTACAGAGTCAGAGTTTGAAAGTGAGAGTTGGAAACTTGACCT
CTGAGTTTGCTGGAGGAAGCCAGCAACATGATTTAGAAAGGATAATATAATCATAATGGCAGGAAGTGAGCACAGTCTTCTTGGCTGGAGAATC
TCAGTTGCAGTGTTTGGGGCAGACTGACTGGAAATGATGCTATGTCGGGGACCATGTTTGAAAGCCTAAAATCTGAGTTGACAAGGAGATGTTC
CATGGAGGCTCTGGGAAAGAGGGACTCGAAGGCAGACCCCCACACAGCGGACTAGTAAGGTCTCGCTCTGTCACCCAGGCTGGAGTGCAGTGGC
ACCATTTCAGCTCACTACAGCCTCAACCTCCTGGGCTCAAGCAATCTTTCCACCTCAGCCTCCTGAACAGCTGGGACCACAGGTACATGCCACC
ATGCCCAGGTAATTTTTGTACTTTTTGTACAGACAGGGTTTTAACATGTTGCCCACGCTGGTCTCGAACTCCTGAGCTCAAGCGGTCTGCCTGC
CTCAGCTTCCCGAAGTGCTGGAGGTGTTTTATGGATTGAGTACTGTTCAGTTGTACCCTATGAAAGTGACCCACCACAATGGCCTGTTTTCCTG
AGCAATTCTAGAGAGACAGCAGAAGGGGCTGGTGGCTCCCGTGAGGCTAGAAGGGCAGAACAGCAGAGGAGGAGAGGGTTGGAACAAAATTGGG
CAGTGGCCTCTGTGCTTGCTGGCTCCCCAGCCCCAAGCCGCCTCTCTGTGGGAACCAGGGAACATTCATACTGCTCGAACGTGGCTCTTCCCAC
AGTCAGACACCACTGGCCAGCCAGGATCTCCCCTCCTGTTGAAAAATGCTCTCCCTTGCAGCTCCCACTAGGAAACCTGGAAGGCCAAACTGTT
TATGACATTGTTTCCCTAAAATGTGCTCAGACACCATGTTTTATAAAGTTTCTGTCTCTTCTTTCTGTTAAGAAAGGAGAAAAAGGATCCCAGC
TACTCAGGAGGCTGAGGCATGAGAATTGCTTGAACCAGGGAGGTGGAGGTTGCAGTGAGCCGAGATTGTGCCACTGCACTCCAGCCTGGGCAAC
AGAGCGAGACTCTGTCTCAAAAAAAAAAAAAAAAAAAAAAAAGGAGAAAAAGGAAAAACATTTCCAGCACTCTTGTCCACCTCCTCAGTTGGAA
GTGTAATAAAAAGATTCTTCTGGCTGGGCATGGTGGCTCACACCTGTAATCCCAGCACTTTGGGAGGCCGAGGTGGGCAGATCACTTGAAGCCA
AGAATTCAAGACCAGCCTGGCCAACATAGTGAAACCCTGTCTCTACTAAAAATATAAAAAATTAGCCAGGTGTAGTGGCATGCACCTGTAGTCC
CAGCTACTCAGGAGGCTGAGACAGGAGAATTGCTTGAACCTGGGAGGTGGAGGCTGCAGTGAGCCGAGATCACACCACTGCACTCCACCCCAGA
TGACAGAGCCACGCTTCATCTCAAAAAAAAATAAATAAATAAAAAATAAAAGGATTCCTCTCTCTAGTGTACCCATGTCTCTCAAGACACTCTC
CCAACTGCCCTGGTGCCCAAACTCGTGTTTATTCCCTTCCTATTCACTATCCTTCACCTCTCAGGTATTAGGTAAAGCCAATGTCTTGGGGAAA
AGTGGCTAAAAAGAGGAAATGACAGTTTTCTTTCTAACAAAAATATGAAACCATCTAAGAAGAAAAAAATGAAGTAGACCTATCTTTTTGTTAG
TGTGCAGAGTAAAAGCCATTTAAAAAAAATTATTTAAAAAAACCATGATTTTCAAAATTCATATAGCTGAGAATACTGTATTTTAAATACAGGT
TAAGTATCCCTAATCCAAAAATATGAAATTCAAAATGCACCAAAATCTGGAGCTTCTTGAGCACTGACATAACACGCAAAGGGAATGCTCAACT
TACTGGTTGAGCAGCCCTATAATGCAAATATTTCAAAATCCTAAAAAATCTGAAATTTAAAACACTCCTGATCATAAGCATTTCAGAGAAGGGA
TACTCACCCTGAATTAACACAATCCATTCAATTTAGCTATTGTTTATAAAATAAAAACTAGCCCAAGCCTTGCTGAGTGGGCATATCTTGATTG
CCACCGTTATTGTTGCAGAGGTACATTGTGAAGTTGCCAGGTCTGCAGGTCAGCCCGTTGCAGGTCCTGGTTTATGGATGGTTGGAAAAGAGCT
AGTCCCATTCCAATTGTCATTGTCAGCCAAGTGCATTGTCAAAATATCCCACTTTCCTGGCAAAGCCCTCATTGGTATGTTTCCACTTGGCCCA
GTGCGTGGAATTGTTTTCATATACTCCCCTTGCTTTGAGAGGTCTGGGCAGAAGAAAGCAGGAGAAGAAGAGGAGTGGGGAAGAGCGGAGGGGT
GCAGACAGGAAAGGGGAGAGCGAGAAGGAGCCTGACCATGTGGGTCTGCCAGGACCCTGCATGGGCCAGCTTATGCCCTGTCCCCTGGCTCCAA
ATGCCCAGGAAAGACTGCCTCGGGAGACGCTACATGTTGAGGTTCACATCACCAAGTGTGCTGTGTCTGAGGCTTGGACCGAGCACTGGAAAAA
TGACCCAGTGGCCAATTAAACTTTCCATTGGCAGACATTGGATGAAAACTTTTTTTTTAAGTGAAATCTTTCCATTTGAGTAGTTTGTTTTTTA
ATGAAATGAAACATGTACTGACATGCGCTCTGTGGAGCAATAGGAGAGTGATTAATTAAACTCTGTGCTGCCATTTTGGAGGGAGGCACTCAGA
GCCAGAGTCAAGCATTAGGAGGCGTTTCAGGGGCTGTAACCGCACCCAGAAGGATCTGTCAGGCCCTTCTGCCTGTTTCCCCTCAGTTCCTGTG
GACTCCTGGAAGGGACAGAGACAAGGGAGATTTCTGCTGGTCCAAGCTCTGTCATTGCTGAAATATGAAACCAAGGACAGTACTAAAAGATGCG
GCAAGGCAAATGCTTTGGAGAGTGCCAGTTCTGCTACGTGATTACACACTCTTCCTAATCTTTCTGGGCAATATGTATGAAGAAATAAAAACAG
TAAATAGCCTCGTAATCAGTTCAGGTTTTGTGGCCAGATGAGGTCAGTTCAGATCAGTTCTGTAGTCAAGTGAGTTACAAAAACTTGATGGTGT
TCAGAGCTCGTTGGATGGTGGAATTGTGGAGAAGGAAATGCTGGCCTCTGTCCGCGTTTATCTAGAGCCGGAGGAGGCTGCCTTTCATTCCCGA
GCATGCTCCAGTTCCTCCCTTGCTTCCAGCTCCCCGCCCCCATCCCCCATTTTCAGCCATTGGAGGAGGATGTAGGCTAATTAGCCACAGCGCT
GCCTCTCACTCCTCCCATGCAACTGAGGAAGTCGCTGGTAAAGGCATGCATGGCACAGAGGCAGGGACCCTGACTCCTTCACCAAACCTCTCTC
CCTAAAATAAGGGGTCGCCCTGACACGTGAGTGAGGTTATTTTACAATAATTGCATTGAAGGCCTACTTCAGTACAGCAGAAACAATATGTGAA
TTTTTATTACCTCAAGAAATGATAATGGCAGAGAATAAGAAAGCATCCTGAAAAGGTCTGGAGAGACATGTGACTGACAGGCTGTGGTTGACAG
CCAGGAGGAGCCAGGATACACTCAGCTCCACAAGTAACAACACAACCAAGAGGACTGGCAACAGACCCCGAAGTCCGAGTGTTCCCTCTGCAGG
GGGACAGGCTGTGCTCTGGCCCAGCGCCTCTGGCTGTGCTGACAAGTTTCTAGCTAACGGGTCGCCACCTCCCCTAGAAGTAAAGGAAGCTCCA
TGCATATGGTGTGTGCTCCTGCTGGCTGTAGCCCTGGGAAATAGGGGAAGAAATCTTGGTTTCTATATGGCTTGACATCGGGTTCTTGCTCCTG
CAAATTAACCGGGTCAGATACTGGGGAGGATTCCAGATGACAGCCTCACCGGCCAGGAAGCAACCAAAGCACAGCACAGCTTGACGGGGAGCTG
GCAGAAGGTGATACAATGAAAAAGGGAAGCAAAACTAGAGAATAGTGGCTGGCAGTAGTTGGGAGGAGGTCAGGACACTGGCCACCCCCTAAGC
ATGGTAGTTCCTGTAGTGTTAAGGCAGCCAGTGTGCAGGAGGCCGCACTCTCTAAGGAGTTAGCGATCTGTCTTCTGTTAGTGCTATGCAGCCC
AGTGTTTCAACAGCTTCCCAAGAGTCAGAAAAACCTCAAATGATGGATGGCCTGGCTGAAGTCATCAGTAGAGATAGGCCCCGGACATGGTCTA
TCTCCCCATCAAATTCACCAAACCTCAGGGCACTGTGGGGTTGCAGGGCCAGACAGTGCCCAGCCACGCTGGCATGATGGCAGATCTCCAACAG
TGGGGAACTAAGAGTTTGCCCAGGCATGAAGAAAACGAATTTATGAAGTTCATAAATGGAGTTTATAAATGCTATAAACAAGGGGCAGCATGCT
TTCATTCTGTGTGGATTTTTTTGTCTGCTTTATTCATACATTGTTGTCAGTTTAATTGAAGTATAATCTACACACAATAAAATTCAACAATTTT
AAGGATGCAGTTTATTGAGTTTTGACAAACATAAACAGTCACACCACCCCATACATAATATAGAACAGGGGTTGGCAGGTCTGGCTCCCAACCT
GTGTTTGTTTTGTACGTTTTTAAAGGGTTGTTAAAAAGAAGAAGACAGGGATCATATGTGACCCGCAAAGCCAAAAATATTTACTGTCTGGCCT
TTTACAGGAAAAGTTTGTTGACCCGTTGACCCTGACAGAACATTCCCATCCCACTTGGAGTTCCCTCTGGCCCCTTGGCAGGGATTCCTCCCCT
CCCCCAGGCAGCCACTGATCTGCTTTCTATAGATGTAGTTGCGCCTTTTCTAGAATTTCGCATGAATGAAATCATACCGTATGTTGCCTGTTGT
GTCTGGCTTCCTTCACTTATCATGTTTTGAGAGTCATTCTGAATTTTTCATGTCTCAGTTCTGCACTCCTTCTTTACTCCTGAGTAGCATGGCA
GGGTTTGACTCCTGGGGGGATGGGAGTACTCCGCATGTTTTAGCCACATGCAGACGCAAAGAAGAAGAAAGGGGATCTGCAAGTGCGGCGGTCA
GGGCCTGGATCCCCGGGGTGCCCACTCTCACTGCGCTTGTCTTTGCAGGCATGAACAACCGGGAGGTGCTGGAGCAGGTGGAGCGAGGCTACAG
GATGCCCTGCCCGCAGGACTGCCCCATCTCTCTGCATGAGCTCATGATCCACTGCTGGAAAAAGGACCCTGAAGAACGCCCCACTTTTGAGTAC
TTGCAGAGCTTCCTGGAAGACTACTTTACCGCGACAGAGCCCCAGTACCAACCTGGTGAAAACCTGTAAGGCCCGGGTCTGCGGAGAGAGGCCT
TGTCCCAGAGGCTGCCCCACCCCTCCCCATTAGCTTTCAATTCCGTAGCCAGCTGCTCCCCAGCAGCGGAACCGCCCAGGATCAGATTGCATGT
GACTCTGAAGCTGACGAACTTCCATGGCCCTCATTAATGACACTTGTCCCCAAATCCGAACCTCCTCTGTGAAGCATTCGAGACAGAACCTTGT
TATTTCTCAGACTTTGGAAAATGCATTGTATCGATGTTATGTAAAAGGCCAAACCTCTGTTCAGTGTAAATAGTTACTCCAGTGCCAACAATCC
TAGTGCTTTCCTTTTTTAAAAATGCAAATCCTATGTGATTTTAACTCTGTCTTCACCTGATTCAACTAAAAAAAAAAAAGTATTATTTTCCAAA
AGTGGCCTCTTTGTCTAAAACAATAAAATTTTTTTTCATGTTTTAACAAAAACCAATCAGGACAGGTGTTTGTTTTTGTTTTCTTTTTTATAAA
TATGAATATATATAATATATATGTCCCTGTACATATACAATGTGGGTGCTAATGTGGAGACTGTGGCCGGCCTGAGCCACCAAGCTGCGGGACC
CAGAGGGAGGATTTTACTGCAAGTCAGCATCAAAGCACCGGTGTTATTCTGAAAACACCAGTGGCCTCATTTTTGGCTTTTGCAAAGCATGAAT
TTTTTCATTTGGATTGCACTTTCCTGGTTCATGACTGTACCTGTAGGTGGTTGTTACTTTGACTCTTTTCAGGAACCACCCCCCAAGCTGAATT
TACAAGTTCTGTTAGCACTATTTGCTTCAACTTACTGCGATTTGTTCTCAAAACTTAAAAATAAGCAAGCAAATGGCTGATACTACCAAGAGAA
CTGGAAGATGGATACCACACAAACTTCTTGTATAAAAATATGAATGCTGAAATGTTTCAGACATTTTTAATTTAATAAACCTGTAACCACATTT
AAGTGATCTAAAACCCATAGCATTGTAGTCATGGCAACCCGCTAAACTTTCTCATGCAACTAAAATTTCTGGGGGAAATGAGGGTGGGGGTTGT
ACATTTCCCATTGTAAAATAAGTGTTTTAAATGTCCTGTACTGCTAACGAATGACTTTCTATATGTCCAGGAGTTCTCCAGTGGAATAACTATG
CACTACTTTACATTTCATGGGGATGCACAAAAACAAAAAAGTATTACATTTTTAGTTGCTGTTTGTACCAACCTTAAATTACATATGTTTAACA
ACAACAAATCAAAAATCCTATTTCTATTGAGTTTTTAATACTGACTAGCAACTCTGAAGTCTTAATTCCTTTTTTGTTATGATTTATTTGTGAG
TTTACATTTTTAAATTGTTTAACTTTCTTAATTTAGTAATTAAAAAGAGAGCATTTTACATTTGAATTTTTTTTTTGTTTATTTGTTTAAATCA
TGGAAGGTACTCGTAGTTCTGTGGCACATCTGTCCCACACCAGATGGTATGGAATCACCTGGTGAATTTAGCCTTTGTGCGATGACTTGCAAAT
GACAGAAACATTCAGAACATCTGGAGAGTTCTTTTCCCTCTAGAAAGGGTAGGCTCTTGACCAGCTATTTCAGAGGATCATTAAAGCAAAAGTA
GGCATACCTTACCATCTGAATTTATCAGACATTTTTTCTAACCGGAATTTACTGTGTCCTGCTTGTAGGAGAAATAGACAATCACAAGAAGAAT
CTTTCTTGTCCTGGAGTCAGTATATTTATTTCCAAATGTCCCCCTACTTCTGTAATGAAGGAGCTAACGGAGCGACAACCTGGTGTGGTAGGAA
GGGCAGTGGGTGGACGCAGGCTACCTGTCCACGCCTGGCTCTGTGATCATTCAGCTTCAGGACCCTGAGCCACCTCCTGGCATCTGTCATTTTC
AGGGCCTGTCTGTAGAACTGAAGGGGTTACATGAGATTCACTCCTTAGATCTCTACAGCTCTAAGATCCTGTAATTCTAAGATCACTGATGCCA
TGCGGGGTCATACAAAGAGGATGGTTTTCTTTGTTGAAATAACCTTTACATTTAGGGAGGGCTTCCAACATTAAAAAAAAAAAGATGAAACAGG
TTCTCTGATTAATCAAAACTTTATGGTATTGTTTTTAGCATTAATAGACTTGCTAAACTGATAAAATTTATTTATTTATTTATTTATTTTTGAG
ACAGAGTCTCTGTCACCGAGGCTGGAGTGCAGTGGTGCCATCTCGGCTCACTGCAACCTCTGCCTCCCGGGTTCAAGGGATTCTCCTGCCTCGG
CCTCCTGAGTAGCTGGGATTACAGGTGTGCGCCATCATGCCTGGCTAATATTTGTATTTTTAGTAGAGAGAGGGTTTCACCATGTTGATCAGGC
TGGTCTCAAACTCCTGACCTCATGATCCGCCCGCCTTGGCCTCCCAAAGTGCTAGGATTACAGGCATAAGCCACAGTGCCCAGCTGATAAAATT
TTAAAGATCTTATACATATATTATTATCACATTATTAGGTAGTTGATTAACATATTCTATATCTTTTTCAAGATAACTTTGCTTTTAATCCTCA
CACCCCACTAAGTTGGATAATTTGCAAATATTTCTGGAGGATTTTTATGTTCTCATTCATTCAAAACAATCTGGAAAAAAAAGAACCTGTTTTA
AATAGAAATTATTTTTAAAAAAAAGTCTCTTCAAAGTATAGCCTTTAATTTATCAGTTGAGTTCATGGTGGACTGTCTTATATGAAAGAAAGCT
AAGGACTTGGGTCACCTCAAGGGCACTCTTAGGAAACAGCGTGCTGTAGAGTGAGGTTGCAGAGTGAAAGTAAGAGAACATCTGTAAATACAAA
GAAGTCAAAGACTTCCAGGTTTTTTCTTCCAAGAGGCAAATGTGCTTTCAAGAGATATTCCTGAGGTAGACCATTCCAGAGAGTCCCCACACCT
CCCAAGAAGCCTCTCTTCCTCATAAGTTGTACAAGCTTAGGGTGAGGAAACGGAATTTGCTGACCAGAAACTTCTCCTGTTAAGAAAGAAAAGT
ATCGCCTTAATGTCAGGGTGATGGTGAGTAATTTTTTGTCTTCAGTAACCTGGCAGTGAATAAGTATTTGCTTTGTTGCCTTAGGAAAGCTGGC
ATCTAGCCCTCCTCTGCTGGAAAGGGAGCCTTCCTAATAAACTATTACTTTTTACTTTTTTTTTAAGACAGGGTCTTGCTCTGTCACCCAGGCT
GGAGTGTAGTAGTGCAAACACAGTTCACTGCAGCCTCAACCTCCTGGCCTCAAGCGATCCTCCCAGCCACACTAGCTAATTTTTATCATGATTA
TTTTTTGTAGAGATAGGGCCTTGCCATGTTGCCGCAAGCTGGTCTCGAACTCCTGAGTTCAAGCAATCCTCCTGCCTTGGCCTCTCAGTGTTGG
GATTACAGACGTGAGCCACTGGGCCAACCTCTTTTTACTTCTTTCAAACAACGGGCCAGATGCGGTGGCTCACGCCTATAATCCCAGGACTTTG
GGAGGCCAAGGCGGGTGGATCACGAGGTCAAGAGATCAAGACCATCCCGGCCAACATAGTGAAACCCTGCCTCTACTAAAAATACAAAAATTAT
CCGGGCGTAGTGGCATGCGCCTGTAATCCCAGCTACTTGGGAGGCTGAGGCAGGAGAATCACTTGAATCCAGGAGGCAGAAGTTGCAATGAGCC
AAGATCGCGCCACTGCACTCCAGTCTGGCGACAGAGCGAGACTCTCTCTCAAAAAAAAAAAAAAAAAAAAAAAAAAAATCAAATAAACCTATCG
GAGAAGACCATTTGCAAAGTTCAGACATTGTAAACTGCGACTAAAGGATTTCTGTTCACTCCATTCTCTGTAGCTCCTGATCTCCTCTGATGAT
CAGTTCACGGGCTGGGAGGATTAGAAAGCCTAGGAGACAGAGGGTCCCATGCTTTAGAACATCAGGGTAGATAAAGACAAATGATGCATTTGGC
TCTGCCGTAGTCGTGACACAGAAAGATGTATGGGGAACCAGGCATTTCCGTGAGAAAAGTTAAGAGTGTATATAATCCCTCAAACACAGCCACA
CTGTTGAAGATTTCCTTACAAGCACCAGAAGGTGTTTACCTGCTTTGAGAAAGATAGGAATCCTCCTGTGGCTAAGTGTTGGCACCAGAGGATT
TTATAGTAGCATTAGTGTGGCAAAATGTTATCTGCAATTTCCCCGGGAGTCTTAAGAATGTTTACTATATTAATTGGAATGGTTCTTTCATGAA
TCTTGAAAGAGTATGGTTTTCAACAACAGGCAGAAGAGAATTGTACTAAAGCCCACAGCAAAAGGGATGTCATCTGTTCATTAGTCATGACTCA
GGACAAAGAATGTCACATGATAGGCATTTCCTAATAAGTTGCATTGGAATAAAATGATCTTTGGTCAGTTTGTTCCTGAAAGGTATTTTAATAG
GTTTAAATGACTTTTGTTGACCTTCTTACCACTATATATTATACAATACTGTTGGAATTCAATTATGCTATCAAAAATATTTGTTAGAAGGGAG
GGAGGAGACTGGTTTTGGTTTGGGGGCAGGGGAAGGAAGCTTAAACACATTTTTATTCTAAAAATGATACAGGGGGATATAGATTACTAAAGCA
ATTGCCAAATTTTAAATATATTTTTTATCTCAAAAATAGTTTATAACTACAGAGAAGAAAATAATGAAAAATAGTACCTTTACTGTATTTGAAA
TATCAAATAGACATTCATAATTTATGTACACCTATGTTCCCAGAAAATTCTCATACATAATAAACTCCTAAATCATACCTCATTCTTTCCCAAC
ATGCAAACTATTCAGAATCCTATGGAGGGACAAATATACCTTTATTTTAAAAGTTAATTCTGAGCACTGAAAATAAAAATAAATGGTTTTCCAG
TGTTGCAGGGACCATTCTAACAAAACGTAACCAGTGACAAACGACGGCTTCCTCAACAGCCAGAACACTGTGCTCCTCTCCATACGGCTTACTA
CTCTCTAATAACCTGTTTAATGCCACCAATGTGTTTAATACTGCTCTCCTCTAATGTGTTTGCCCTGCCAGACTGTGAGCCCGAGAACAGAAAC
TTCCTGATTGGTTCACCATTATTCTCCCTGCACCTAGAACAGTGATGAGCTTCTGGCAGATGCTGGCTGGACTGATGGATGGACTGTTAGGTGG
ATGAGGCCCACTGTCCAGTGGGGCCAGTGGTTTACCTTCGAATGACACATCTACCTAAGAACCTGCTAAGCACATGGGACTGACTGCATGCTGC
CCACAGTGCTAGCCCTGAGCATTATGAATGTGTGGCCACCAGTGTTCCAGATATGATTGAAGTCCCAATCTATAGCCTGAAGTGAACACCCTTG
ATACTTCCCAACTCTTTCCCCAGAACTGTCACAATGCCAGTGAGGATAAACTTGTACCTCTTGGAACCTGAGAGACAGCATGAAGTAAATGGCA
CCAAGTGATACATACTCTTATTTTGATTCCGTTTGGTTTTGAGATGGGGTCTCACTATGTTGCCCAGGCTGGATTCAAATTCCTGGCCTCAAGC
AATCCTCCCACTTCAGTCTCCCAAGTAGCTGGGACTATAAACATGTGCCACCACCCCTGGCCACAGTCTTTGTTTTTTGGGGATGCGGGGAGGC
GACAGGGTCTCACTCTGTTATCCAGGCTAAAGTGCAATGGTGTGATCATGGCTTACTGCAGCGTCGACATTTTGGGCTCAAGGCATCCTCCCAC
CCCAGGCTCCCAAGTAGCTAGGACTACAGGTGCATGCTACCACATTCGGCTACATTTTTTTTTTTTTTTTTTTTTTTTGGAGAGACAAGGTCTT
ACTATGCTGCCCAGGCTGTAGCCTTAGGTAAAGTGCCTTCAAAGGCTCGTTTTAGCTTTGTCATTTTATGAAAAGTTGATATTCTACTCCCTAA
GATATCCATCTTTGTCTTCAGATAAAAATTATTTTCCCAAGTCATCAAGTTGTGCCATGTTACCCAGCCTGTGGCTTGTAGTGTCCCTGACACA
CCCTTGCATAACGCAGGGAGCCCCTGTGCCCTGTGTGAGAAGCACAGCGTGAAGATGTTGTCCATAATATTGTACATGGAAAAGGTAACGTTAG
CCCCCATTAATGTGGATTCTCTCATCTAATTTCCTCTTTTCACCCAGCAAAGTGCCCAGTGACTGAGAATGAGACAGAAGATGAGGGCGTTTGA
GGATGAGAAGGAAGAGGAAAGTCATCAGAGCCACCTGGAAGTAGAGGTCTGGTCAGGTTGGCCTGCTTAACCAAGATGGCAGCCAGGGGAGTAG
GAAGGATGAAGACATTGCACACATTTGGAGGAAGAAACCAAAGCTGGCATGACAGGAGTCCTTTCCAGGGCTGTTCAAGGCAACGGGATGCAGG
TGGATATGACTGTAGATGGGTGTAAAGACTTACCATTCACTCCTCCAAACTTTCGTTGTATACTTATTATGGGCTAGGCCCTGGGCCTAGCCCC
AGAAATTCAAAGACAGACAATTAATATCTACAGTCTAGTAAGGATAAAGACCCATAAAAATCGTTGCAATAAGCTTTCAGATGGAAGCACAGTT
GTTAGCAAAGTAGTAACATCTAGCTCAGAATGGGGGGGTACAGCGGAGGCTTCCTGGAAGAGGTGCTGCCTGAACTCTCTTAAAGACAAATGGA
AGTAGGTTACATAGTGTTTAGCGGGGCCTGGTGCTCCTTGCCTTTGGATTTGTTTCCTTCTGATTTCTAGCCCCCATTAGGGATTACCTCATGC
TTTCAATATGGAAAACATCAACAAATGGGTCTCAATGAAGAAAAACCAAAAGTACACACAAGCATCTCAATCACTGTCTCTCTGTTTCTCTCCT
CTCTTTCTGTCTCTCTCTCTCCTCTCCCGCCACCCCATTAACAGTATATAGGTACACCATTTATTCTGATTAGTGGCCACTAAATAGAGGGGGT
GGTAGATAATAGCATTCAAGGACGGAGGAAGGTTTTAGCAGGTTGTTGGCTCCAGTGTCTCGATTAGGATATGGTCTGAAATATATTCACATTC
TCATAAAGTATGAGAATAAAAGAATTGCTAGAAAGAAGCTTGGAATAATAATAACTCTTCACTACTTCTCTTAATAACAGCTGGTGGGAGGCTT
TCTCCTATTTACAAATCCATTGTCATTCTTCCACTTACTAGCCAGGCTGTTTCGCTTGAATCATCGTCCGATATGTACATTCATCATGGAAGGT
ATATACTAGCAGTTGCATGGTGTTCTGGTTGTTTTGTTCTGGGTAGTTTTCCTTTAGTTAATTATCAAGTGCTTACTATGTAATAATCAATGAG
TAGCTAATTGGAAGCAAGAGAGAATGAAATGGGTATTATTCAGATAGTGTTTTTAATGAAAAGCATAGACGTAGTACTGTGGGGAAATATCACT
AAATAGCAAGAGATTTCATCTGTGTGTGTGTGTGTGTGTGTGTGTGTGTGTGTGTGTGTGTGTTTACAAAAGGATTCATTTAAGCCACTCATAA
ACTAAATCCAGGTATGTGAATCTTTTCTTTTTTTTTAGACTCCTCCCAATGGGTAAACATTGGAAGACACCAGGAGGGAAGCCAAGCCTCTAGA
GATTGCTCAGGAACCCAAACCACCCCAAATGGAGCCGGAAGGTCTGTCAATTCCCAGAGAGGGAGGAAATGGTAAGCAAATGGCAGCAGTTGCT
TAGAAAATTAGGGAATGAATAACAATGCTTTCTGGTAAATTAACCATATTACATTGTGCACCGGAAACCGATAGGAAAACCCTGTTAAAAATTG
AATAATAATTTGCCCACATCATTTATGCAAAATGCATAATTTTCCTACTTCAGTGGACTTTAGTTTGATTTAAAAGATGAGCAGCTCTAGGACT
ATTCACTAAAGCATAATTTGTAAGATAATTGGGAAGGGGAAGAGAAATCAGACTGTAATCAGCACAATCTTTCGTAAGTCACTTCACCTTCCTC
CTCTCACCTGCAAATTTTGGAAAATATTTGTTTACCCTTTCCTGAAGTTCTCTAAATGGAAGTGCAAAGCATTAATCATTGTCATTGCTACCAC
TAAACCATGACCTCACCATGAGAGGGGGGAAAGCCATGTTTCATTCTATTCAGTATTTTTCTGCTCTAATTCAAATAAGTACTTGTTTTGAATA
GTGGGGAAAAAAATGGAAATGTAGCAAAGCTATAGTTAAAACAACAAGCCGAGAACAAAAGCAACATGCATGCAGGCAAAGGAGACCTTGGAAG
ATACACAGATAAAACAAAAAGCTGTGCTTGGCTGGGATTCTGTTTCAGAATCAAATGCAAAATGGAGAATATTGTTCCAACTGTAAAGGTTTTG
CAATCTACAATCAGTCAGAATGAAAAGATATCATGTAATCCACCTTAACAGTCAGAAGCTGCACTATTGCTGTGAATTATGATATTTGTTCATG
ACTAATGTAGATTTTCAGACCCAGAATGGTTGAAGGTGGGCAGGAATTTGTAGGTATACCAGATACCCTTCACTTCAACAGGGCACACCCATGT
CAGCTGATGTTTATATCTCTTCCTCTCCTCCTTTTCCCTCCTCCTCACCCCTTGCTCCCCAATCTCATTAGAATCATGAAATTTTTACTTATAT
ATTCATTAATTCTCTTCATCCCCATCTCTCCACAAAAGATCATTATCTACTTATTAAATTCAGCCCTTACACAGTGTCTCATGCCTGTAATCTC
AGCTATTCGGAAGGATGAGGCGGGAGGATTGCTTGAGGTCAGGAGTTCGAGAACAGCCTGGGCAACATAGCCAGATGTCATCTCTAAAAAAATT
AAAATTAAAAATTTAATAGGGCACGGTGGCACACACCTATAGTTCCAGCTACTCAGGAGGCTGAGGTGAGAGGGGATCACTTGAACCCTGGAGG
TCAAGGCTACATTGAGCTGTGATTGTGCCACTGCACTCCAGCCTGGGCAATAAAACAAGACCCTGTCTCTGAAAAAAAAAAATTTTTTTTTTTT
AAATAAATTCAGCCCTTAGAGTTCTACCTTTCAGGGCAAACCTGTTTCCTTTGTTGGACTAATTCCTGTCTCTTCTACAAACTTGCTGGTCCCA
AAACGCTGAACTTTGAACCCCACAAATCTCAACACTTCTTATCGGTTGGGTGCCCATTCTGGTTAAGCAAAAGGACCTTCCCTAATCCTAATAC
TCGTCCCCACTCTTTCCATCTCTATCCCCAACCGCTGCTACTGGCTGAAGATTTATTAGATACTAGTTTTGTACCTATGCGTCCTGCCACTTGC
TATAAATCGCCTTTATGCAAATCGAAAAAGCTACCCTGCCTCAAGACACTTTGCTGCCATCTGCCAGGAAATTGTTGTAATAAATATGCAAAAG
GATAATGACTAGAATGTGAATGGTGCCCCTTAGAATTGTGCAGTACGCAACTGGCACAACTATATGTGACAACCCTGTTGTTTGAAATCACCTG
AACTCTCCTGGAACCCTGTGTAAAAATCACAACCACCATAGGTCAGTTTTGCCTTTTCATGACAGTTTGCCATTTCAAAATCTAAATCTTCCAC
CATGATCAGAAATTGCTGAGGGGCAGTTTCTAGGAGAGACAGAGTCCCCACGCAGCAGTAACTATAAGATTATAGGGCTCTTCCAGCATTCAGC
CCAAGCTTTGACCTGCTACCTGGGCAGATACAACCCAAACCATACTTGGTTGATTTTCGGGAAGCATCTAAGTTAGAAAGCTGGTAAGGCTGAA
TGTTGGGGTGCAGGGGGAGGTAGACCTAGCTTACATTCAGGTCAGCCATTACCCATCTCTGGCTTTGGCGGCTTCATTTCTTGTTGATTGCCCC
ATCTGTTCTCCTCCCTCCTTCCCATTCAGCACAACCTGCACGTACTGGTGATCTCTCACTACATAACAAACCACTCCAAAACTTAGTGGCAGAA
ATAGAAACAGT
HUMAN SEQUENCE—mRNA
CGCGCTTCTCCCAGCGCACCGAGGACCGCCCGGGCGCACACAAAGCCGCCGCCCGCGCCGCACCGCCCGGCGGCCGCCGCCCGCGCCAGGGAGG
GATTCGGCCGCCGGGCCGGGGACACCCCGGCGCCGCCCCCTCGGTGCTCTCGGAAGGCCCACCGGCTCCCGGGCCCGCCGGGGACCCCCCGGAG
CCGCCTCGGCCGCGCCGGAGGAGGGCGGGGAGAGGACCATGTGAGTGGGCTCCGGAGCCTCAGCGCCGCGCAGTTTTTTTGAAGAAGCAGGATG
CTGATCTAAACGTGGAAAAAGACCAGTCCTGCCTCTGTTGTAGAAGACATGTGGTGTATATAAAGTTTGTGATCGTTGGCGGACATTTTGGAAT
TTAGATAATGGGCTGTGTGCAATGTAAGGATAAAGAAGCAACAAAACTGACGGAGGAGAGGGACGGCAGCCTGAACCAGAGCTCTGGGTACCGC
TATGGCACAGACCCCACCCCTCAGCACTACCCCAGCTTCGGTGTGACCTCCATCCCCAACTACAACAACTTCCACGCAGCCGGGGGCCAAGGAC
TCACCGTCTTTGGAGGTGTGAACTCTTCGTCTCATACGGGGACCTTGCGTACGAGAGGAGGAACAGGAGTGACACTCTTTGTGGCCCTTTATGA
CTATGAAGCACGGACAGAAGATGACCTGAGTTTTCACAAAGGAGAAAAATTTCAAATATTGAACAGCTCGGAAGGAGATTGGTGGGAAGCCCGC
TCCTTGACAACTGGAGAGACAGGTTACATTCCCAGCAATTATGTGGCTCCAGTTGACTCTATCCAGGCAGAAGAGTGGTACTTTGGAAAACTTG
GCCGAAAAGATGCTGAGCGACAGCTATTGTCCTTTGGAAACCCAAGAGGTACCTTTCTTATCCGCGAGAGTGAAACCACCAAAGGTGCCTATTC
ACTTTCTATCCGTGATTGGGATGATATGAAAGGAGACCATGTCAAACATTATAAAATTCGCAAACTTGACAATGGTGGATACTACATTACCACC
CGGGCCCAGTTTGAAACACTTCAGCAGCTTGTACAACATTACTCAGAGAGAGCTGCAGGTCTCTGCTGCCGCCTAGTAGTTCCCTGTCACAAAG
GGATGCCAAGGCTTACCGATCTGTCTGTCAAAACCAAAGATGTCTGGGAAATCCCTCGAGAATCCCTGCAGTTGATCAAGAGACTGGGAAATGG
GCAGTTTGGGGAAGTATGGATGGGTACCTGGAATGGAAACACAAAAGTAGCCATAAAGACTCTTAAACCAGGCACAATGTCCCCCGAATCATTC
CTTGAGGAAGCGCAGATCATGAAGAAGCTGAAGCACGACAAGCTGGTCCAGCTCTATGCAGTGGTGTCTGAGGAGCCCATCTACATCGTCACCG
AGTATATGAACAAAGGAAGTTTACTGGATTTCTTAAAAGATGGAGAAGGAAGAGCTCTGAAATTACCAAATCTTGTGGACATGGCAGCACAGGT
GGCTGCAGGAATGGCTTACATCGAGCGCATGAATTATATCCATAGAGATCTGCGATCAGCAAACATTCTAGTGGGGAATGGACTCATATGCAAG
ATTGCTGACTTCGGATTGGCCCGATTGATAGAAGACAATGAGTACACAGCAAGACAAGGTGCAAAGTTCCCCATCAAGTGGACGGCCCCCGAGG
CAGCCCTGTACGGGAGGTTCACAATCAAGTCTGACGTGTGGTCTTTTGGAATCTTACTCACAGAGCTGGTCACCAAAGGAAGAGTGCCATACCC
AGGCATGAACAACCGGGAGGTGCTGGAGCAGGTGGAGCGAGGCTACAGGATGCCCTGCCCGCAGGACTGCCCCATCTCTCTGCATGAGCTCATG
ATCCACTGCTGGAAAAAGGACCCTGAAGAACGCCCCACTTTTGAGTACTTGCAGAGCTTCCTGGAAGACTACTTTACCGCGACAGAGCCCCAGT
ACCAACCTGGTGAAAACCTGTAAGGCCCGGGTCTGCGGAGAGAGGCCTTGTCCCAGAGGCTGCCCCACCCCTCCCCATTAGCTTTCAATTCCGT
AGCCAGCTGCTCCCCAGCAGCGGAACCGCCCAGGATCAGATTGCATGTGACTCTGAAGCTGACGAACTTCCATGGCCCTCATTAATGACACTTG
TCCCCAAATCCGAACCTCCTCTGTGAAGCATTCGAGACAGAACCTTGTTATTTCTCAGACTTTGGAAAATGCATTGTATCGATGTTATGTAAAA
GGCCAAACCTCTGTTCAGTGTAAATAGTTACTCCAGTGCCAACAATCCTAGTGCTTTCCTTTTTTAAAAATGCAAATCCTATGTGATTTTAACT
CTGTCTTCACCTGATTCAACTAAAAAAAAAAAAGTATTATTTTCCAAAAGTGGCCTCTTTGTCTAAAACAATAAAATTTTTTTTCATGTTTTAA
CAAAAACC
HUMAN SEQUENCE—CODING
ATGGGCTGTGTGCAATGTAAGGATAAAGAAGCAACAAAACTGACGGAGGAGAGGGACGGCAGCCTGAACCAGAGCTCTGGGTACCGCTATGGCA
CAGACCCCACCCCTCAGCACTACCCCAGCTTCGGTGTGACCTCCATCCCCAACTACAACAACTTCCACGCAGCCGGGGGCCAAGGACTCACCGT
CTTTGGAGGTGTGAACTCTTCGTCTCATACGGGGACCTTGCGTACGAGAGGAGGAACAGGAGTGACACTCTTTGTGGCCCTTTATGACTATGAA
GCACGGACAGAAGATGACCTGAGTTTTCACAAAGGAGAAAAATTTCAAATATTGAACAGCTCGGAAGGAGATTGGTGGGAAGCCCGCTCCTTGA
CAACTGGAGAGACAGGTTACATTCCCAGCAATTATGTGGCTCCAGTTGACTCTATCCAGGCAGAAGAGTGGTACTTTGGAAAACTTGGCCGAAA
AGATGCTGAGCGACAGCTATTGTCCTTTGGAAACCCAAGAGGTACCTTTCTTATCCGCGAGAGTGAAACCACCAAAGGTGCCTATTCACTTTCT
ATCCGTGATTGGGATGATATGAAAGGAGACCATGTCAAACATTATAAAATTCGCAAACTTGACAATGGTGGATACTACATTACCACCCGGGCCC
AGTTTGAAACACTTCAGCAGCTTGTACAACATTACTCAGAGAGAGCTGCAGGTCTCTGCTGCCGCCTAGTAGTTCCCTGTCACAAAGGGATGCC
AAGGCTTACCGATCTGTCTGTCAAAACCAAAGATGTCTGGGAAATCCCTCGAGAATCCCTGCAGTTGATCAAGAGACTGGGAAATGGGCAGTTT
GGGGAAGTATGGATGGGTACCTGGAATGGAAACACAAAAGTAGCCATAAAGACTCTTAAACCAGGCACAATGTCCCCCGAATCATTCCTTGAGG
AAGCGCAGATCATGAAGAAGCTGAAGCACGACAAGCTGGTCCAGCTCTATGCAGTGGTGTCTGAGGAGCCCATCTACATCGTCACCGAGTATAT
GAACAAAGGAAGTTTACTGGATTTCTTAAAAGATGGAGAAGGAAGAGCTCTGAAATTACCAAATCTTGTGGACATGGCAGCACAGGTGGCTGCA
GGAATGGCTTACATCGAGCGCATGAATTATATCCATAGAGATCTGCGATCAGCAAACATTCTAGTGGGGAATGGACTCATATGCAAGATTGCTG
ACTTCGGATTGGCCCGATTGATAGAAGACAATGAGTACACAGCAAGACAAGGTGCAAAGTTCCCCATCAAGTGGACGGCCCCCGAGGCAGCCCT
GTACGGGAGGTTCACAATCAAGTCTGACGTGTGGTCTTTTGGAATCTTACTCACAGAGCTGGTCACCAAAGGAAGAGTGCCATACCCAGGCATG
AACAACCGGGAGGTGCTGGAGCAGGTGGAGCGAGGCTACAGGATGCCCTGCCCGCAGGACTGCCCCATCTCTCTGCATGAGCTCATGATCCACT
GCTGGAAAAAGGACCCTGAAGAACGCCCCACTTTTGAGTACTTGCAGAGCTTCCTGGAAGACTACTTTACCGCGACAGAGCCCCAGTACCAACC
TGGTGAAAACCTGTAA

TABLE 22
MOUSE NOMENCLATURE
ICSGNM Nfkbil1
Celera mCG15940
HUMAN NOMENCLATURE
HGNC NFKBIL1
Celera hCG22180
MOUSE SEQUENCE—GENOMIC
CCTCCCAGGCCTTGGGAATACAGGAATGAGCCACTGATCCAGCTTCAGTTCCCAACTATTAAATCCACCACAAGGGGAAGGGTCAGCCAAGGAA
CTGAAAGTCTATTAGTTTTTTGGCAAGGTATAATGACATACAGCCAGAGGCACAGGCACACAGACATAGACAATGCCTCCAGATTACAATCTAT
TTTCAAAATCCTAGGAAAAAGATATATAAATACGCTAAAATGTTTCAGAGGCCAGTAAGCTTGCACACAGACAGGTTAGGAAAACAATGTCTAA
TGCTTGGTTATGCCGAGCAATGGAAACCTCAGAGATCACAAGATCTCTGGCCTCCTTTAGCATTTTGCACATACTGCCTTTCCTTTTCTTCTTC
TAGAATTCCCCCTTTTAAATCTGTCATGCTGGGCGGTTGTGGAGATGCTCGCCTTTAATCCCAGTAGAGGCAGAGGAAGGTGGATTTGACTCAA
AGGTCAGCCCAGACTGATCCAAGCTGCTCTTTCCCCACCCTAACCAGAATAGGAAGTCTGCACACATCTCTCTACTCACAGGGCCAGCTTACAA
GGCCTTTCAAACCCAGCAGGCCACTCACCCAAGCATGGAAATATATAATCTCAAAAGTTCCAAAGGATGCCAACTGTGAGGCCACACCACGTTC
CTTCACAGAGGACACAGGCCACTTGTTATGACCCCCGGAAAGCTTTAACCACTCTAGAGTTAAAGTCCACACCAGGGCTGTTTTAAGAGCATCT
AGCTTCCTCTTGCCACATACAACCCAACTCTCAAACAATCTACCAAGCAATTCAGGAGAACAGAACAGAGGCTCTGGCCAAGCCAGGTGGCAAA
GGCCTGCCCCAACTACTCAGGAACCTGTGGCAGGAGCCTCAGGTTCAAGGCCTACTATCTCTAGACTAAGGTGAGTTCAAGGCCTACCAGGACA
ACGTGGTGGTTAAGAGACAGTCGTGAACATAAAAACAAAGGACCCAGGGGTGGCTCAGTCCCACACATAGTAGCTCAAGGCTCTGGAATGGAAT
CTGCAATACCAAAACCAACTAACCACAAAGTGTTGCGGTGGTATCACCTAAAGCTACGAACTCTCAGGAACACACAGGTGTATTCACAGTATGG
AACCTGGAAGGGCAGCCACAGAAGGGTGTGGCACTAACTGAAGTCTCTCATGTGACCACACCACTGACAGCACAGGGATGCCCGAGGACAGCCA
ACCTCCCTCCCAGTGCCTGTCTCCCCACAGCCTCCTGTCCCAGGTTTCTTTCCCCCCTTACCTTGACATTCGGCATGTACTTAGAGAAGCGCTC
ATATTCCTTGCTGATCTGAAAAGCCAGCTCCCTAGTGTGACACATCACCAGCACAGACACCTTTGAGGGAGCAAATGGAGACAGACACAGTAAA
GTCAGCCCCTCGTCACAAGAGCATCCAGCAACTTGTCAGTGATGTTGGTGAACATTCCCCTTGAACTGCAGAGGACACCAGCTGAGAGCACATG
GAGCCAGAAATGGCCTCCTGGTGATTCAATCTCCACACCCTCTCCAGCACTGCCCCCAACATACCTGCCCAGTAACGGGCTCCAGCTGCTGCAG
TGTGGCCAGGACAAACACTGCTGTTTTTCCCATGCCTGACTTGGCCTGGCACAGGACATCCATCCCCAGAATGGCCTGCGGGATGCACTCATGC
TGGACTAGAAGGAGAAAGGAGAGACATTTTTGCACCATGCCAAACCCCTTTCTCCCCAGCCGACTCATCATATGTCACCTTGTCACCTTTCAGA
CCCCATCCTTTCCCTGACCCTCATCACTCTTACACTCAAGTCAAACCCTCCTACCCCAGCTCCCCAGTTCCGCTAAGAAAGAACCCTCGAGAAG
CTACCAACAGGCCACCTTCCACGTTCCTGGCTAAACTTACTTTTCTTACACCGTAACAATCTATGAATTCGACTGCCACAGGCCATTTCTCCTA
CATAGGTTGCTGTGCTTTTGGGTCATGTAACCCTGCAACCTTGAACAAAATGAAGGACTTGGTACCTGACCCAGAAGCCACAGAAGCCAGCATC
CTGATCGTTAAGGTTTCCGGAAACGACACTTGGCCTGGCCCAAAAGAAGGGAACTAAAGGTCCAACCAGGCCCTCTCTGGGAGTTCGAACAGTC
AGAATGACAAAAACCAAGCAAAAAAGCCAGGCACAGAACAAATGGCAGCCCTCGGTTTAATGGAGAACAAAAGCCTCTTCTACAGCTGCCGTCT
TGGGTACATTCTCAGAGCTTGGCCTATCCCAAATAAATTCAAATCCTATGACCCTAGACTTGAACCTCCCTTTTCTTGCACCAGAGGTGCTAAA
TAAGGTCTCTACACACAACAATAAAATGTACCCTCTGATGGATGCTCAAAGCCACAGTCAACGATGGCCCGGAGCAGCTCTGGCTTGAGTAGAA
AATCTCGGAAGCCGGAGCTATGGATGGAGACGTAGGAGCCCTTGACGTCTTTCTTGGCGGGAGCCTCGGTCCCATCTGCCCCAGCGGCTGTCTC
CACCTCGTCGTCTTCGTAGTCCAAGAGCTCATTGTCCACATCGTTCTCTGCCATAATTGGGCCGGCAGGGGGAGAAGGGAGGTCTCTGAGTTAG
GTGTCCCAGAGCAGGTGAGGAAGCAAGGGCCAAAGAGAGGGGTTGAAGAACAGCAAGGGGAAAGACACCAGTTTCCGACAGAAGAGCTGGGAGA
GGGGACAACAAAAAAACCGGAAAAACTCATCAGTCACGAATGGACTCGTCACTTCTCGTCGATCCCTACACCCGTCCTATTCTTCCCTAAACTG
TGAAACCTTTGTCCCGAAAGAAGAGTTAACAAAGAGTCTCATGACATAAGAAACGGGAACACCAGACGGGAGAGACAGAGTGGGCTCCACCGCT
AACCAGACTTTGTAACACACACTTCGACAGGCAGGAGCCAACTGGGAGACCGCAGTAAAATAAGCGTGACTAACGACTCACACGAGGGTTGAGA
CACAAAGGACAAGGGAAGACAGAGACTGCGCTTTCTCCTGCCACCTCACGGTGGTGATCCCCAACGTCAGCCATCAGTCAAGGGTGATAGATAA
GGGTCAGCACTGCGCAGAGCGCTCACTTCCCAAATGCAAACATCATGTGGCTGCCGCTATTCTCTTGCTCAGAAACCCCCGAGTGGCCGTCCCA
GCGGCGCACACCTGGACCGGGGAACAGAAGCAGGAAGGAGGGTGGTGCGGGGCGGAGAGTGGGAAGTGTATGAATGGCTCCATTTCAAGAAAAA
AAAATTTTTTTTTTGTTTTGAAAAAGATGGGTGTGTGCAGTTCAATGGCAGTGCTTGCTTAGTAACGCGAGAGGCCGCAAGGTTCGGCTCAGCG
CTGCCAAAGTTAATTTAAAAAGTCGCACTAAGGAATTAAGGTCACAGCTGAGAACGATGCAGGATTAAGAGTGTGAGCTGGGTGGCCTTAGAAA
GGAACGCCTTGCAGTGCTCTCGGAAAAGCTTCCCCAGAAGTGCGCGACACCATCGGCCACGCACAGAGGCAGCCATTCACAGAGAGCTCCAAAT
AAGGCAGCGAAAGCGGGGTCAAAGGGCTGGACGGGCCCCGGAGCGAGATCTTCCCATCCCTGGGCGAGCAAGAAAAGGCCCGGAGGGGAGATGC
GAGGGGCCCCGCCCTCCTCCTGGCGCCGCCTCCGGCCGCCGCGGCCCCGGGTGGGCAGAGGACCCACTGGAACACCCAGGAAGCGCGGAGACAC
AATGGCGGCCGAGGCCACCATTTTGGATCGTTCCCCCCTTGTCTCCCAGCTCCCCGCCAAGATCTGCTCTCAGAAAGGGGTGAAGACAACAGAA
CTGGACGCCCGCCTGCGCGCCGGCGGCCACGGCGCCGACCCGGCACCGGGTATCAGGGACCCGCGCGGCGGGGCGAGCCGCGGGAGAAGCAGAT
GGCGGTGGATGGTGCACAGGGCCAAAGCTTACCTAAGCACTGCGAGCGTGTATGGCGGCAGCAACGGGGACGGAGGCGGGAAGCTGCCTTCACT
TCCGGTTCCAGGCTCGCCCCGGCCTCCCGCCTTCCCGGCCGCCAGGGCGCACGCGCAAGGAGCCGGAAGCAGGCTTTCGCCGCGCGCGCTCCGC
GGCTGGCTTTGCAGAACGCCCACTTGAAATGAAGCTTCTGACGACGCCTCCTCCGGTCGCCAGTAGCAAGAGCGCGACAGGAAACTCACGTCTT
CCCCCGCAGAGCTCTCCATTTTGTTCTTCTACGCTTATGCAACACCCTCCGACGGTCGGCTCTACTTTAGTTTTGTTTGCCACGCCAGGCTTCT
CCCTGGGCACCTCTCTGTGGGCATGGAGGACTCCGCGATCGAATGAGAAGCGTTTCTCACGGGGTCCTTAGCCTCGTAGTCAAGGTGACCCGAG
AGCGGCTAACTCAGGCCTCTGCTGGAGGGGAGGGGGACACGTGACCACGCCCATTACAGTCGTGCAGAGAATAGGCAGGATTTACCCAAAATTA
TATCGAAAGATGGAGGCTTACATTCTGTCTAGGTGCTAAATAAATGGTGGGGTTTGAGGTTATGCACCTTTCCATAATTCTTGCTAACTAGAGC
AGCAGAATAATCTCAGTTCCATTATTCAGCCTGGACATCCCCCTCCCCCCATGTGGTATTCCCTTTGAGATGTTACATATAGCAGAAGTGTTGT
CCTGTTGCCTTGCTACATAAACACAAGCTCGGGCTTTCCCTATTAAGGACACTTAATATTAATACAAAGTAACACTAAGTATCAGTGTTGCCAA
CCAGCTGGCTGTGTCCTGCTCATCGCATAAGCTCTCTGGGCTGGGGCTTGGTAGCTTTTGATCCCTCCGCATTTGGCAGCCCTAAGCAGCAGGC
CGGCGGGTTTTGTATAAATCAGAGAGGAGTGGGAATTCCTCGGCTCTGGTAACTTATTACGAACTGTAGCTCCAGAAAGTGCGGTACCTGGGCC
TTTAGCTTTGAAGCGTTTCCAGGAACTCTCCTAGTGACCTAGTTGGCGGGCTCTTAGCCACTGTCTTCCTGTTACTGGACCTAATGTGGTGACA
TTGTGGAGGGTAGCATAGTAAGGTTTGTCCCAGCTGACCCAAGTGAGCCAAACTTTCTGCTTTACAGCCAGCAAAGGGTGTTTGCACCAAACAC
AGCAAACAGCAAACTCGTGCAAATGAAGACTTGTGTTTGCACCAATCATAATATTGTCCCTTTGGCATCAGTGATAACACATCTTCCTGCTACT
GGACTATGGGAAGTTCCACCCTCCACTGGGGGAGGAGAGAGAGGACAGAAACAATCCTGGCTGAGCCATTAGCTGTGTGGTTTCCTCAAAGACT
TTATAGAAGATTCAAAATGGCCATGCTGACAGTCCTTGTTAAGGTAGCCAGGGCAGGAAGGGATTGTCTTAACAGCTGGCTGTGATTTTAACAA
TGCTTAAAACCTGGGCCACTGGGGCCACAGCAAGGTGAGTGCCCCTCATCTGTCTTGCCATTTTTTTCTTCCTTTCTCTGTTACTGAGTTTGAG
CAGTTATATAGACTGGCCTCAAGCTCTCAATTCTCCTGCTGGAGGATTTGAATGCAGACCCATGCCCCGCTCCACTCTTCCTGTAAACATATGA
AAATAGAGAAATAAAACAAGGTGGCCAAGGCAATACAGCTAGCAGGGAAACACCAGGGTGTAAGCACCCCAATTTCCTATCACATGTGTCCCTC
CTCTTCAAAAATCTCCTTGCTTTTGTATAGCTATTTCCCCCGAAACATTTCCTCCTGATGAGAGTGGAGAAGGCTCAGGAAATTCAGAAAAAAA
GTTGCAAGACACGGAACACTTCTCCCAGATTGTGTCACCAATAAACAGCTCCTGGTGGCAGGACTCTTTGGTCTGCATACAAGTCACGCTGGTG
GGCGACTTTCTGATGGGAAGCTACTCCAGTCTCACTCCTATAAGTAACCTATCACCCACCATACTGCCAATAACTCCAGCGAGCTCTTTGGTCT
GCCAAGTGAGACTTGGGTGGTGAGATATCTTTGGTGTTTCATGACTGCCTTATTTGGCATAGATTTTTTTTTTTTCCTGAGACAGGGCTTCTCT
GTGTAGCCCTGGCTGTCCTGGAATTCACTCTGTAGACCAGGCTGGCCTTGAACTCAGAAATCCACCTGCCTCTGCCTCCCAAGTGCTGGGATTA
AAGGCATGCGCCACCACACCTGGCTTATAGATTTTTTTTTAATGTCTCTCTACAGAAAAGTCATATAAGACTATGAATTACCTCATTGATTCAA
GTTCAGGGTGAAATGAGCAGTGAAGAGAACTGATAAAGGAATGGGGGTCAGAGACCAGTAGTGTCCCACCCCCTCACCCCCTACCACCACCACC
CCAACTTCCTCCTTCCCCACCCCAGCATCTTTTTATTCCAAAGTATTCCTCAACTGCCAAGTGTGTGCCAAGTGCTATCTAGGCTCTGGGGATA
CAACGACCTTTATCATCAGATACATAATGAGAGCCAATACCCAAACATAATGTCAAGCCAGGCGTGGTGGTGCACGTCTTTAATTCCAGTACTC
AAGAGGCAGAGGCATGTGGATCTCTTGTGAGTTCAAAGCTAGCCTGGCCTAGCAAATTCCAGGGTAGTCAGAGATACTTAGTAAGATAGTAAGT
CTCTGTCTCTCTGTCTCTCTGTCTCTCTGTCTCTCTGTCTCTCTCTCTCTCTCTCTCTCTCTCTCGTGTCTGTGTGTGTGTGTGTCTGTCTGTC
TGTCTGTCTGTCTGCCTGCCTGCCTGTCTGTCTGTCTCTGTCTCTCCTGCTGATGAGCAGTGTAAAACCAGGAAAGGGACAGAGTGCTGCTCTG
CCAACATTTCCCTTACAACTATCTATGCAACTCTCTGTTCAAGAGGGGACACAGTACTGTGTCCTTTTGTTTCTCCATAAACCTTCCAGGACAG
CCCTTTGCAGTGTTTTGGAAACTATATAAATGCATAACTCTGAGGACAGCGAACCAAAGTGAGCAACGGCAAGGCAAAAAGCAGCCTCCTTTCA
CCGACAATTGACTCATCCACCAGTATTTTATTGACACTCCGCTGTTACACAAGGAGGCAAGGTGCTGAGGGCACACAAAGGAATGCATGCATGT
GAATGTCATAGATATCTTCAGCTCCCGGAAGCAGCAAGATTTGGGAAAACAGAGAAAGAGGGGGGCCAACCATGAGAAAGACCAGCTCAGGAGA
AACAGAGAGGTGCTAAGAGATTTGATATATATTTGGGACTCGGACAGAGGGGCTAAATAACATTATCATTTAACTGTTCGGGGGCATGATGTGC
CAAGGGAGAGCCAGAGAAACAAATTCTTTAGAGGGTTACTAGTGTCACAGTGGCTGAACAAAGCTGGAACACGAGGAATAAAAAAGTCAGGAGA
GGGGGTTAGAATTCTGGCAGGCACGCAGAGTGCGGTCATGATACAGGCACTCCGTCATCTGGAACTATGCTGAATTCTCACAGATAATTCACAG
CAGCCACCTTACAAAGTGAGCGTTGTTGCTGTCATTCCAGTCACAAAGATGAGGACACTGAGGCTTCCAGGTTAAATAGTTGTCCCAGTTGTGT
AACCAGCAAGGACCAGGGGTGGGAGAGGAACAGAGGTTCCCCTGCTCTGAAGTCAGGGTGCTTTCATAACTGTCACCAGGACATATATTTCACA
GAGGGGTCCGGTGGGGATGACGTGGGCATTCATTAGTAAACTTCCAGTGGGTGTCATGGAACAGAGAGATTCGGGTTTGTCGGGTTTGAACAGG
CTACTTGTAAGTCTCAGGGGGGAATATTGGATCCTGTCACATAGAAGCCTCCCCTCCAGCAACAGGTTATTATGTGAAGCGATTTGGTGGGTGG
GGGGGATGATATTATGGATTGAACCCAAGGCTCTGCGCATGCGACACAAGTGGTTTTGAGTTGGGATATTTCTCTGTGGACGGAGCTGACAGAA
GGAGTCACTCTAGGGATGTGTCCCTGAGCGATGGTTCTAGACAGTAACCCGATAGTTGGGGTCGACCTGCGGCCTGACTTCACAGACCATGCCG
AGAAGCTGAGCCAGGACGCGCTCCCGATTCCTCTGCTGGGAACTCTGCATGCCCTGAACCTGCCGTCTTGTGGCCTGCTCCACTTCAGCAGACA
GGTTCCCCTGAGAGCCCATGGCCTGGGGGATGGGGGCGGAGGAGAGAAAGAAAAAAGCAGACACAGAGAAAGGAGGGTCCCAGCATGTAGGAGG
AAGGGTTCAGAGGAAGGCCTGGGAAGCCTTTTCGAAACTATTCCCACCCCACAAAAACATGTAGATACCAAGCAGGTGGGCACAATCCTTACTT
CTGAGACTGACCATAAAACTGTCCCCACCATACAGACTGTGAACGCCGCCCCACAAGTCTCATGTACCCTTTAAAACCCTCAGACCTTACAGAT
CCCGTCAAAGCTTTACCAATGTGTGGTCCTTGTCAGAGCCTCTTCAGCTCTCAGACACCCTAATTTCTTAGTCACCTGTATACACCTGGCAAGA
GGCACCCACCGATTTGCACCTGGGTGGGGCCATCCCTGTCCCCCTCAACTCACCGCCTGCTGCTTGCTCTGAAACTCCTGCTCCCGCTCCCTGC
GGTATTGCTCCACCTCCATTTGAGCCTCCTCCTTCGCCTGCTTCAGTCGCCGCGCCTTCCCTAGAGGCAGAAGGACAGTGAGTGTACACACACA
CACACAACCAGGTGCCTTAATGGGATCAGTGCCCTCTAGAAAGTTATCCAGTTTCCTGTCAACCAACCAAATGCTAGACTGTAAAACCCACCCG
TTTCTTCTACCCTAATAGCCTCAGTCCCCCATCTGCCCTCATTGTCCTTGGGCTTTTTTTTCCTAAGGACTCAAATGCCCGCTATCCTCTATTC
CTAAATTCTCCTTCCATATTCCTAAGAGAGGAAAGGGGGGCTCACTCTTCCTGGCATCGGCCACCTTCTCCGCTGCCCGCTTCTCAGCCTGGAG
GAGCTGCTGGATACCCTGGGTCTGACTGGCCATTTTTATCACTAGAGAGGATGCTGTTCCGAAGGCGACCGAGGTACCAGACGGCTCCCACCCT
CCAGCGCCTACTCCTCCGGGCCCTTGATGCGCTCCTCCACTCCCACTGGGTCTTAGCGCTCCCGTTTTCACTCAGTCTCCTGCACCGGGAGGGT
GTCTGTCATCCTCACACTGATGACTGGTCCTCCTCTCGAACCTCACTGAGGTAGTACCCGAAACGGCCGCCCGAGGGCAGCAGAGACCAGCCCT
GTGCTGACTACTGCAGCAGGTGCAGGGCCTCTCCGTGTCACCAGGGTCCACAGAGATGGGTCTGGGGGAAGCCGAGGGCAGAGTGTTTCGGTGT
GAGATCTTGATTGGTGGTCTCGAAGGAGACACAAAGGCGATGAGAATGGTGTGGGAAGGTAAGGATGCGCTTGGACACAGTACGCTGGGTTTCA
CTTACACTCCCAACTGTGTCTCCACGCCTCCCTCCGCCCTTCCCCCCTCTTTTCAGCCTCCATCTTCTCTTCAGACTTTCCCCCCAAATCCGAA
CGTCTGCACACATCTCCCTGCTCTGTCCTACGTTTGCCTTGTTAGTGTCTAACCATGTGTTTTTCTTTTTTGATCTACCCATATATTTGGCCTC
ATTGTGGTTGTGTGGTGTGTGTGTGATTATGTGTGTGTGTGTGAGATTGTGTGTGTGTGTTTCGAGACAGGGATTCTCTCTGTAGTCCTGGCTG
TCGCTGCAGCTCGCCCTATAGACCAGGCCTGCCTCTGCCTCTGCCTCCCTTCGAAAGCTCGCCTCTCATCTTCCGCTTGCAGGAGGACCCTATG
TCGCAAATTGTCTTGTTTCCCCTCCGGAGACCTAATCACAAGTCACCCAGTCCGAGCGTTCGGTACGATTTCCGGGATGGAACACACAGTTCAT
TTCCGCCCGAAACTTGCGCGTGTGCTTGCGCCATTTCCTGCATCCTGGCGCGCCTCCTCCCTCCCCACCTCCCTTCTCCGAGCGTCTTAAGCCC
AGGCCTCCGGCCTCCGTCTCTGAGGGTCCTTGGGGGGGGCGGGGAGCAGGTCTGATGAGTAACCCCTCCCCCCAGGCCCCAGAGGAAGAAGCCT
CTACGTCTGTCTGTCGGGTACATTATATTCCATTTCTAGATCGCTCTTTGAATTTGTGACTTTTCTCCAGATTTGTGGCTAATGAAAATTGCCC
CCATCTGGCCTTACTTGATTTTTGTTGTTGTTGTTGTTTCCCTCCAAAGTTCAGAATTAACTTTTTTCTTGGGCTCTGGAACCTTAAAACAACT
ATGTATCATTTTCTCAAAAATTACATCAAAAATTAACTCTGTGATTCCCCCCTCCCCACCAGCCCCCAACTCCAACCCCATCCCCACCCTGCGC
CCCAAACTTGCCATCTGCTAAGGGATGAGAGTTTAGAATAGGCCTTATCTCTTGCTGGAGATGCAAGGGGGAGGAAATGGGAGGTAAAGGGAGA
AACGGGGAAGAGCGGAGCTGGGACCTCACCGCTGCCATTTCCCACTTCTTCCTCAGCCCCAGAGTTGTTCCATGGCCTCCGCTTCCCGCCGGCA
CCGCCGAGAGCGACGCTTCCGGCGGTACTTCTCTGCAGGACGGCTGGTGCGAGCCCAGGCTCTCCTCCAGCGACATCCCGGGCTTGATGTGGAC
GCTGGGCAGCCCCCGCCTCTCCACAGGGCCTGCGCCCGCCACGATGCCCCTGCCCTGTGCCTGCTGCTGCGTCTCGGCGCTGACCCTGCCCACC
AGGACCGCCATGGCGACACGGCGCTGCACGCTGCTGCCCGCCAGGGCCCTGATGGTGAGTGGGTCCGTGGGAACTGGTCTTGAGAAGCTGGCCA
GGTCTGAACTAGAAAGAATGCGTGGGAAATAAAGGCCTGGTGTGGTGGCGCAGGTCTTTTGACTCTGGGCAAGCAGAGGCAGGAGAATCTCTCT
AAGTATGTAGCCTACACAGTGAGCTCCAAGACTACAGAGAGACCCTGTATCACACAACAAAATAGTTGGGAAATAAGCTAGTTCTTTGGTCCTT
GGGAACTGTTGAAGATCCCTGGTTGGGATGTGGCCATACCTCTCTAGTAACAGAAAAGCTTATCCTAACATCGCCCAATCAACTAAGGGGTGTG
TGCTAGCGTGGTGTGATTGTTTGTTTGAAGATTTATTTATTTTATGTATATGAGTACACACTATAGCGGTACGGATAGTTGTGAGTCTTCATCT
GGTTGTTCGGAATTGAATTTTAGGGCAACCGCACTCTCTCCTGCCCATATTTATTATTATAAATAAGTACACTGTAGCTGACTTCAGGCGTGCC
AGAAGTGGACGTCAGATCTCAAGACAGATGGTTGTGAGCCACCATGTGGTTGCTGGGATTTGAACTCAGGACCTTCAGAAGGGCAGTCAGTGCT
CTTACCCACTGAGCCATCTCACCAGCCCGATTGTTTTTGTTTTTGAGTCAGGGTCTTACCGAGTCTCTCAGGCTGGCCTCTGCATCCCAAGTGC
TGAGGTTGTAGGCTTGTGTCTCCCACACCCAGCCGCATAGCTCGCAACTCTTTCTTTACATTTCCTAGCTTTCAGCTCAGTGACTACTTTGTTT
CTTTGATGGTTTTTTACCTTGCAGTTGACTGCTCAGGTCAGATCCCAGCTCCACTCACCCCACCCCCACCCCCTTAGTTTTGCTTCTCTCCTTG
TAATAATGCCACAAACACCTTACAAAGTATCCATGCTTTGTCACATGATCAGCTGGGCCGGCCAGAGGACTTGAAACTTTTACTTGGTGTTATG
CCGGAGATCTGCTGCTGTGTGTTAGGGATGGACTCATGTCCCTTGACACTTACCAGGCGCCAGACCAGTTTTCTTCATTCTCACCAAAATTCTG
TAACAAAAATCCTACTTAAGCTGGTGGGTGCGGTCTGGAGGCGGAGGCAGTCCGGGCTCTGCAAACTCAAGGCCAGTCTGCTCTACATAGTGAG
TTCCAAGCTAGCCGGAGCCACGGTAGAGAGACGTCAACAAAACATCTCCAGTCAGCATCGGTAGTGGCTCCACAGTGCAGACAGCCTCTTTCTA
GTTCATTTGTGTTACTTACTGGCAGATAGCCAGCCCAGAAACAGCCTCCCCGCCTAGAAAGACAGGAAGTTCACAACACACTCCTGTAATCCCA
GCCCTCGGGAACACCTCAAGGCCAGTCTGGGCAGGGCCTAAGAGTCTCAAAAAGTGAAGGAAAAAAAAGCCATCAAGCAGATCTCACCTAACCT
ACATGGCGGCTATGGTGTGACTAGTTTTATCCCGCTGAGGTTAACACTCAGGGGTTTCCTACCAGAACCCTGCACTCTCCTGCCCCTGCTCCCC
CAGCCTCTTGACCTAGCCCCAGATGGCATACTTCTAGATCAGTTTTGCCAAATCCCACAGTTCTGGAAACAGCGTAGGTGTTGCCTTTGCAGTG
ACAGCAGAAGCAGTTCCCTGGAGAGCAGCTTCCAGACTAGAACTGGAGGAGAGGCCCGTCTGTTAAGAGACGGACGGCCTTCTGCCCACAGATC
AACAACCCTGCTCACTCACTCATCTCCAGCTTGGCCTCTCCTTGATCAAAACCAAAACCAAACCCAAAATCCTCTAACACAAATAAGATGGGCC
TGCGTAGTTGATTTTCTTTAAGCTTAAAGAGAGGAAGGCTGCAGCTGCCTGGCTGAGTTTATTATGGGATACCTGAAGAGAAGAAGTCCCCAGA
CTCCTGAATACATCCTGTGGAATCTTTATATACCACCAGTAGGGCTGTTCCAAGCAGAGTAGCAAGCCGGAAGCAGCCTCACTTCCAGTTTGAG
GTTTTAAGAGCAAAAAAAATGCAATTGTGGCGTATACAGAAAGGCAGCTCTGGACAGCTGCAGCAAGCAAGCTTTGCTTACAGAAGCAGAGAGG
TAGCGGTTAGATGATCAGGCAGTTAACCTTCAGCTCAGAGTCACACTGTACAGGAATTTATGGCTGGACCAGAGACCAGTTTACTCTAAGAGCT
TATAGCCGTTCAGTTCACCCAAGGTTGTTATAGCACTTAAAGTATTCTTAGGTATGCTTTCAGGAGATGTGTACCTAGATAGAGTTCAGCAGCC
GGCTACAGTTTAAGAGGCAGCTCCTGGGTGGTAGCAGTGTGGTGCCTAGGGTTGCCGCTTACGGATCCTGAGGCCTGCACCCTTACCTCGGGAG
CTTGTTCGCCTCCGAAACGGAGGTTGTGATAACACATGAGCCACGGACTGGCTGCAGCTTTGCTACGTAATGATCAGTTCGTGCGGTTCAGTTA
ACTTGGCTGAGGATCTCCTAATGGTGGAATAGGCACCGCCACCGTCGTATTGAATTCTGAGAAACATGTGAAACATTTAACACACCGTGGCGAA
AGTGAGGCCTCCCTGGGGAGTTTCGGGAACTTGTCTGCATGTCACAGTTGAGAGGTTGCAATGGTCCAAAGAGGAAACCATCCCTCCACACTTC
TCCCAACCCCTGGTCTTTCAGACCGTTTCCCGGCACCTGCCAGTCCTGCCACGAGGTGGCGCTCTAATCCCAACTTCATCTCATTCAGCGGCTG
CCCGCTGCTTTGAAGATAGTTGAATGAGATTGATCCATTGAGCAGATTGAGCGATTGAACCTGCAGGAGTCTCCCCTCCACTCTCCTGTTAGGT
CAGAGGGCTGCCCTAGGACACTGAAATGACTGGGGAGCGGGGGGTACCACACACAGTAGGGGGCCAGAAGAGGCTCACATTTTAGGCTTTGTGT
GTGTGTGTGTGTGTGTGTGTGTGAATGTCATGGAAGATCAGTTTCAGCTGTCAGAGGGACCCATCAGTAAAGCATGCGTCAAACAGTGGCCACT
CTGCAAAGAGCAAGCATGAGTGGACACTGGCAGGCAGTATAGTTCTCTCCGTTTTTGGGGGGAGGGAAAAACAAGCTATCATTCTGATGTTAGG
GTCTGGGGTTTAAGGTTTGCTTTGGTCTTCTTTAGGGTTTGGCTTGCTGGTTTGCTTGGTTTTTTGTTTTTTGGGGGGCAGGTTGTTTGTTTTG
TTTTTGTTTGTTTGTTTGTTTGTTTTTACATAGATTCTTTCTGTAGGCTTGTCTCTTCTGGAACTCAGTATGTAGATCAGGTTATCCACAAACT
CAGAGACTGATCCGGTCCCTGAGCCTAAGTCCAGGGTTGCAGGTGTGAGCCCGCACCAAGCCTCATCCCCAGCCTTCGTTTTTCTCTCTTTAGT
GGAGACACCACCATGTAGCCCTGGCAGCTCTTGACTGATATTCCTGACCAAGACGCAGGCCTGCACCTCCAGGCTTGTTTTGTGGTTATGTGAC
ACTCACTGTGTGCTGATCCGTCAGCACGGCATAGACAGCATCCACTGCCTCCCTGATCTGCGCAGTGGAGCCAGAGACGGCCTTGTTTCAGAAT
GACTGGGCACAGTTGATGTTCCACCTATCCTTCCAGCCTCGCATTCTGCACACCCACAGACCTGTCACCTCGGCCGCAGTTGTCTTCAGTGACC
CTAGCACAGGTTTCACAAGCCTTACCCCGTCAGGAAGTGGGTTGTGAAAGAGTTTACCAGAGCCAGACATGGTGGCATAGGCCTGCAATCCCAG
CACCCAGGAGGCAGTGACGAGGATTACAAGTTCAAGGCCAGCCTTGGCTACAAGGCCATCCTGAGCTACAAGAAACCCTGCCGGGGTTTGGGGT
GCCAGAAATAAATAGAAGGCAGACAGTAGAGAGGAGCTGTCTGCACTTGGCATCAGGAAGTGTTTTCACACCATGGTACCCTGGTCGTTTAGGT
CTCGAGGGAGGGACACTTGTGTGGGCTCATGTCAGATGCTCAGCTGGATATTCCTCTACTGTGGGATACTTGTCACAGTGTAAGTTATTTCTTA
GAGTCCAAAGTTGTCATGAGTTACCAGGGGTCAGACCCTGATGGGCTGAGCCTGCTTGTTAGAGAGAATTCTCTGTAGTGCTGTTGTGAGATCG
ACTGCAGCCTGGCTCATTGAGCTTTGTTCTTCTAATCTTAAAATTTAGTTTTGTTTTTCTATCTGTGTGTAGAGGTCAGAGGATACAGTTGTTG
GTCCTCAACTCTCATTGTACTTGAGACAGAATTTTTTTTAAGATTTATTTATTTTATTTATGTAAGTACACTGTTGCTGTCTTTAGACACACCA
GAAGAGGGCATCAGATCTCATTACAGATGGTTGTGAGCCACCATGTGGTTGCTGGGATTTGAACTCAGGACCTCTGGAAGAGCAGTCAGTGCTC
TTACCCGCTGAGCCATCTCTCCAGACCATCTTGTTTGTTTTCCAACCATGTCCTCCAGGCTAGCTGGCCCATGAGTCTCTATGGACTCCCAACT
CCACCGCTTATCTTGCAGTGGGAGTATTGGGATTGCAGATGTGGGCTGCTGTGGCTGGGATTGCAGATGTGTGCTGGTATGTGGGATTTCAGGT
GTGATTGCTGGGGATGCGAACCTGAGTCCTCAAATTTCCGTGGCAAGCGCTTTACCAATATTGTGTTCTTCCAGACCCCAAATTAACTCTTTCT
GGTCTTGCTCAAAGACAGTAGCAGTCTTGCTTGATCCCAGCCCTGGGCAACCAGCCATCACAGAACATCACCCGCACACCCCTACACATGAGGC
TCACAGAAGATGGAGGCCATCATCTGGAGTTGCATCAGGGCTGAGTGCGGCCCTTGCCCACAGTGCTGATACGTCAGAGTCATGAGACCACAGG
ACATCAACTTTGTTGGAAAGAGGAGGTGGAGTTACTGTTGAAGGGGACCAGAGGTCAGAGGTCAGGCTGTGACACCAGAGCAGGGGCTCCTGGG
CGGTGGGAGGCAGCATCAGTGTTTACGTAGTGGCTGCTGTTCCTGAGGCAGCACCATCTGGAACTGAGAGACCTGAAATCAGGGAGACATTGAT
TTACCATAAGGTTGAGTAAGCAAGGGCCAAAGACACACAGCAGACTTAAAACAGAGGTTCCTGAGAGCTGAGGGAGTCCAGACGCCTAAAAGAG
CTGCTAGGAGCTGCCCCACGGAGGCTGGTGAAACCCCCGAGTTAGACAAAGCAACAGCTGCTTTGTTTCTCACTGGAATAAAGAGATGAGAAAC
ATCTGAACACCAGGGAGGCAGCACAGACTACGATCATGCAGTTCTGAGACTTGCAAATATTTGTAAGTTACAACAGGAAAGCTGTGAACATGGA
AGAGCTGTCCCTGGACAGGACCGTGTGTCACCTTGTGTGTCATAGCCATGTGGCCTTCTCGGGAGGCTCTGAGAGTCTCAGACCCAACCTTAAG
ACAATAGAATACAAGCAAAATAATGGTGAGTGTCCCCTCGTCCAAGGCCAGCATCACACTGGGCCTGTGGGGAAATGGAGGGCATTAGGGTGTG
TCTGTGTGTGTGTGTGTGTGTGTTCATGTGCGCGCCGCACCAGCCATTGGGATGGAAAAGAACCCTCTCTCTGTCTAGGGAGCCTGAAATGTTG
GGGTAAAGGTGGGACTACCCAGATAGTTTAAGAGGTCTCCTTAGGGAAAGCCAGAAAGAATCCCTTGAAATGAGCCCCTCGGTGCCTGGAGGGG
GTAGGAAGACAGAGCTTGAATTGGACAGCGGGCTTCCCAGTGCACCACTGTCGCTCTGGCAGAAGACCCAGGTTCAGTTCCCAGCACCCAGGGG
ATCTGGCCCCTCTCCTGACCTCTGTAAGCATCAGGTACCCACGTGGTGTGTATACATAAACGTTAGGAAAATGCTCAGACACATAAAATCTAAA
AACACATTTTTTTTTTAAAATGAGTTTCAAAAATTCTATCATTAGAGCTCAGAAAGGAAGAGAAATTACCTCAGCTGCTCACTAGGATCTGGAT
GTGTTTAAGGATGGCTTTCACCTGGTGCTCCCCAAAAGAAGAGAGGGCTCTCCAAGCAGTGTGCAGGAGTAGGCTGCTAGGCTTTGACTAGAAC
AACCCTACCTCCAGACACAGAACAGAGGCCTTACAGCATGGCTGCAGTCTGAGAAGAAAGGAGCTGACAGGCCTGCCCGTCCACTTAACTCTGA
AGGCTTTTGTCCTGAGAGAGGGTTGGTTTCAGGTTGTTTGTTTATATGGCTGTTTGGTTGGTTTTTGAGACAGGGTCTCTCTGTGTAGCCATAG
CTGTCCTGAAACTCACTCTGTAGACCAGGCTCGCCTTGAACTCAAAGATCTGCCTGCCTCTGCCTCCTGAGTGCTGGGATTAAAGGCAGGAGGA
AGGATTTTTATGGTAAATCCAGTTCCCAGGTTGTGTGAATGTGAAGTGCTCCCAGTCAGACTCGGAGCCGTACTCACCCTGGCACCCAGTGGCA
TAGCGCACAGGGACAGCACAACCATGAAGGGCTTGGAAGGGCCGAGTTTGGAGTGCAGCCTGGTGAGGTCCCTTCCTTGTTAGATTTGTCCCCA
CCCCCTTTCTACAAGATGGAGGGCACAGCAGCAAGGAGGTGGCACTGGATCCTGAAGGCCTTGCTTGTTCTGAGTGACACTGGGAATGAGGACC
GGGTCCCTGACAGACTGAAGGGAGCAGGGAGTCAGATCCAGGGCTCATATCCCGAGACAGTGGAACCCTGGGATGTTGTTTGTTTTTACCACCC
AGGATCTCCTAGACTTGGTGAGCAGGGTGCGTGTGCGTTAACATCTGCTTCTAAGTTCTCCCTGCCGTGGTCCTACGCTGCAGACTCTAGAAGG
GAGGGGCCCTGAGACCCAGGAGGAAGGAATTGAGGGATGTGTATCAAAGGCAGGGCACAGAGCAGGCGGTGCAGGCTGAGCGGGCTGAGCCCAA
GGATGAGTATGGCAGCCCAGCTATGCTGAGACAGCGTCTTGGGGACATTTTTATGGCAGCTTTGGGAAGGTTNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNAGAAGAGGAAGATGGGACTTCCCCCGGGAGGAGGTGGCTGATTTTGTTTTATGCTAATATTTATCATCATCTTCTTCTCCTTCA
TCATCATCATCTCTTATTTTTGTTGCTGTCTTCTGAGACAAGGTCTCTACATAGTCTCGGCTGTTCTGGAACTCATTATGCAGTCCAGACTATC
CTGGAATAAACAGATCCTCTCACCTCTGCCTCTCCAATGCTGGGATTAAAGATTTGTGCTCCCACACATGACTTGAAAACAAATGAGCAAAATC
CTAGTTTTGTTAAAACTCAGTTTCAAAGTAACCAAAAGCCCGACAAACTTGGCAGAAAACAATCTTCATGGCCAAGACAAAATTTCTGTCACTT
GAAGGAGCTTTTGGTCAATGCTCCAGGCAGCATCTCCCAGACGCTAGTGTGAGGCAGAGGTTGGCCTTCAGCGTGGGTGCGCATGCTGCACTGC
ACCGGGTTTCACCGGGTTTCGGTGCCCCAACCAGCTCACCCTCACCTCCAAGCTGCAGACCTTGTGGCTGACGCTCTCACGTGTCAGTTTTACT
CTCGTCTTAGCTTTTCCGTTTTGGAAAAGTCCAGTCATTTCACCTTAGGAGAATCTTAATTTACACATACAGACCTTCTTCTTAACTAAAAATA
CCCTTTTCCTTTCCACCTTCCTCACCAAGAGCGCCTCTCCACACTCCATGCCTCTGCGTCTCCTGTACACTGTCTCATTTTACTCTTTACTTCC
TGACCAGAGCCTTGTCTGTTCAGCTGCATCCGGGACAAAAGGTCTCCTGGCATCACTGCAGACATGATAGCTGACTAGGGGGCGCTTGTCTGTC
CAGAGTCTTTATAACTGCTTGGATTTTTTAAAAAAGCTCTTTTTATGCTTAAAAAAAAAATGTTGAGATAGAGAATTACAGAGTATTCCATTCC
TGTAGAAGCTGACAAGGATAACAATTGACCTTGGGCGGTAATTGTCTCAGTTTAAGCCCCAGACTGGAGACATGGCCCACGGTGTTGCAACAAT
GTTGTGTGTTAACCTTGTAGTTGCAAGTTTTAGATTCTAGATGCAGTTGTTCCTGCCCCTCATCCCCTCACCCCCCCCCAAAAAAAAACAAAAC
AGAAAGAAATAGCAAGTTGTTGGGTTTCCTTGGAGGACCAGGGGGAGAGGAGAAAAAGAGAGCGGGATGGAGCAGAAGGGGTGGGGTGAAGGCT
GTTCAGTGGGGGCTGGAGATTCTGTTGGGTCCAGTGATGGCAGACTCCGTCTAGAGCAGTTGTGTTTGGGGCAGGTCCATCAGCGGGAGGAGAC
ATAGACCAGATAGTGTTTCGGCCTATGTCAGCTGCCTTGTCCAGGGCACAGATGTCAGGGTGTGATAGTTGTTCTCTGGATTTCCTACCTGAAG
TCTTTGAATGTGTGTGGATGAGAGGGAGTGCCGTTGGAGGGAAGAGTCAGAGGGGAAGGTGGGGTGCAGTCGCCAGGTGGAGGGATAGGTGCTG
GTCTCGGAGAAGCTGGTGGTTGAGTGGGACCTGGCGTGGCCATCAAGTCTGTCGCCTAAGGCTGAGTTCAAGGGAGGCCGGTATGGGAGGCACC
TGTTCTTGTCCCGTGCTGAGGCCATAAAGGGGGGATTTTTTTTTTTAATTAAAATAACACCAAAGAGATCCAAGTGTAGGACAGGAGTGAAATG
GTGTGTTGTGCTGTGGCCAGCCTAGTCTGGCCAGATTTCATTACAAAGGAAAATAAGGTTTTGTTGGCTGTGTCTTTTGAGAGGTTGAACATAG
AGTTTAAAGAATGTGAGTCAGAGGTCATGAAACCCATCATACCAGAGATGCGCGCTCACCCAGAAGGCCTCGGAGGGCAGCAGGCCTCTTTCCA
GTACTGACTTGACTGAGGTGCCCAGAACCAGGGCATCCTCTGAGAACAGAGACCTCCTGGTCCATGGGGTGAAATCCAGTTGGTCAGGCCAGAC
TGACCTCAAAGAGAGACACACAAATAGCTGGCCAGAAAAGGGGGTTCACTGCCCCTGAGAGAGTGAAATTTGGGCTACCCACGGGTCTCTTTAT
TCAGAAAGTCTGGCTGGGAGGAGGGAGGTGAGCAGAGTTTCTTATATGGTTGTCTATGGTGCTGAGGTTCCAGCCCCCAAACCTGTCCCTTTTT
CCGTGTGGTCCCTCCTCCCTCTGAGTGGAAGTGCTGTGCTGTTAAAGGTGAGGAGAGACTTACACTTTTGTCATCAAGGAAAGAGTATTTCTTC
TACAGAACTTTGCATATTCGCTTCCCTGTGTGAAAGGGTGCTCCGGCAAACATGATGGCACAAACCTCGCGGGAAGGTGGGCAGGAAGAGAAGA
TGTTAGTGAACATTAAACGCATTGATTTAGGGGCTGGAGAGGCAGCTCAGGGGTCAAGAGACCACAATGCTCCTGCAGAGGACCCAGGTTCAGT
TCCCAGCACCCAGGTTGGGCAGTTCGCAACAGCCTGTACCTGCTCCCCGTGAGAGAGTACCAGGACAGACGGGTATGTAGAGACCCTTGTATTT
TTCTGGCTACTAGTTATATTTTTCTGGTACTTGTTATATTCAAGTACACTATAGCTGTTTTCAGACACCCCAGAAGAGGGCGTCAGATCTCATT
ACAGATGGTTGTATTATTTTGTGAGTAACCTGTTCATGTCTTCATTTATTTCCTTTGGAAATTTTTTCATATTTGTACATATATGAAAATACTT
TTTAAAAAGTATTTAATCCTTAGGGCTGGAGAGAGGGCTCAACAGTTATGAGCACTTGACTGCTCTTCCAGAGCTCCTGAGTTCAATTTCCAGC
AACCACATGGTGGCTCACAACCATCTGTAATGGGATCTGATGCCCTCTTCTGGTGTGTCTGAAGAGAGTGACAGTGTGCTCACATACATAAAGT
AAATAAATACATCTTTGGGGGGGTACTTTAAAATTTATTTCGTAACTTCACTCCAGCTTTAGGAAGGTAGGTGCTGGCAGAGGCCAGGCGAGGA
AATGAGACTCCCAAAGCGGCTCTATTACCCACACATCTCTCTGTTCAGAAAAGTCTGGCTGGGACAGGGAGATGGGACTGGAACTCTCTGTAGA
TCAGACTGTCCTGAAACTTAGAGAGATCCGCCTGCCTCTGCCTCTCTGCCTCTTCCTACCAATTACTGGAATTAAAGGCCTGTGCCACTATGTC
CGCCCTAATAAAACCATTTTTAAAGATATATTTATTTATGTATCATTTATTTTATGCATACGAGTACACAGTAGCTGTACAGATGGTTGTAAGC
CATCATGTGGTTGCTGGGAATTGAACTCAGGACCTCTGTTCACTCTGGCCCAAAGATTTATTTATTGTTATATTCAAGTACACTATAGCTGTTT
TCAGACACCCCAGAAGAGGGCGTCAGATCTCATTACAGATGGTTGTGAGCCACCAGGTGGTTGCTGGGATTTGAACTCAAGATCTCTGGAAGAG
CAGTCAGTGCTCTTAACCACTGAGCCATCTCTCCATGAAGGGTTGTCATGCATGCATGCACAGTAAAGCCCAGCAGGCTTGCACATGTTCCGAC
TTAGAACACCCACCCTCGGCAGATATGTGTCCATGTCCACACTCGTGCAAGTTTAAGGCTAAAGGCTTTGGAGACACAGTCTGACAGACAGGGT
AAAGAGAGGTCTGTGTCTCAGCTCACAGTGGAACACTCATTACAGTCATCAAAGAGTGAAACAAACGGCAGGTTCCTGGGGGAAAATAAAAGAT
AAAAAGCCATGAGGAGGGAGAGCTGTTAGTTCACCCCAGCCTGACTCCACAGTGAAACCTGTCTCATAAAAACACCAAGATGAACAGCTAGCTG
CTGCTGTCAGCCTGTGTGGGATGTGTATGCCCCAAGGATTCTGTTAGCTTCAGTTGGATTGTGGTGACCCATAACAAGGTAGGAGAGTCACGAG
GTTGAGACCTAGTCTGCAGTTACAAAATGAGCTCTAGGACACCCTGGGCTGTGAGACCCTCTCACAACACACACACACTCACACATACACACAC
ACACACACACACATACCATAGCCAATAAAATAAATCGGGTATTTCAAAAGAAAACAAAAACAAAATGAAGCCAGTTGCCAAGATAGGCAAATAC
CTCTGAGTAAATGGGCATAGTGTGGCCACAGCTCCAATAACTTGCCGTGTGCACAATTGTATGGAGCAAGGTAGGACTCGAGGTTGCAGCAGAC
TGTTAGCAGTTATGATGTTAAGCTGGATATGTTGGTATACACACATACACACACAAAATAAAAAGACAGAGTTAAATCTTCCATATGAGCCTGG
GCCTACTCAGAACATATAAACACACACATAAGCACACACTATGCACTCACACACTCAAACACACTCTGTACTCTCACATAAATACACTGCATTC
TCACACATAAACTCACTGCCTCACACTCCCATAAATACACACTATGTATTGACACACTCATAAACACACACTGCACTATTGCACTTGCATAAAC
ATACATTGTGCACTCTCACACACAAACTCTGCACTATCACACATAAACATACACTGTGCACACATAAACACTGCACTCACATAAACACATACAC
TTGTTAACATAAACATACTGCATGCTTATACACACATAAACACTCAGCACTCACACACACATAAACACCACACTCACATACACACATAAACACT
ATGCATGCTCACACTCACATAAACACACAGTGTGCTCTCACACGCTCCATACACAGTCACACCCTGCAGTGAGCCCTAGTGGGAAGAAGTTGAA
GGACGGAAGCCAGAAGCAGGTCTGCTAAGTGGGACAGCTCGGGAGGGTTTGTGGCAGATCACAGCAGGAAGCCTGCCTTGCCCTGATGTGGTTT
CAGAGTGGATTGGCACCTTGGCCTGCTGTGTAGGCCCTTCCATCCTGCTGGCAGTTACAGGTCAGCCCTGCCTCTGTGCCAGCCTCCTGGTAAG
TGAGCTAACTTCCTGGAACCAGCAAGGCCTGTGTCCCATTTCCTTCTACTCTAGGAGAGGAAGTAGGGCCCCGGCCCAAATGCTGCCCCAGCCG
CTCGCCTCCCTCTGGCTCCTGATGGAGGCAGGTGGTGGTCCCAGCTGCTGGGCTTCCTTCGGTAGGGTGAAGCGATGGCATCTGGCTCCCCTCA
GTTTACCTGGTGCTCAGGCTCAGGAGCAGGCACCCAAGGTGCTTGGTCCTTCCCTGCCCTTGACCACCATCATTTAGGAGGGACTCACTCTGCG
CTGGCAGCACCTGTACGTCACTGCCACCCTTCCCTCCCCTGGGGAAGGCTGGAACATTCTCCTGAAAACTTTTATTTTACTCAGATTTATTGGG
TAGAAACAGTAAATGTACTACTATGTTTGTGTGTAGTGGTGCAGAACACAGTGTTTTGATATTCTTTATTATTATTAATTTACCATATTTGGAC
TTTTTGAGACAGGATTAATCCTGTAGCCCTGGCTGACCTGCAACTCACTCTGTAGACCAGACTGGCCTTGAACTCTCTACCTCCTGAGCACTGG
AATTAAAGTGCCACCATGCCTAATGTGATAATCTTAATATGTAATTTTAGATGAGAACAATAGTTTGTAATATGTATATAGTGATAATAGCTTC
AAAATAAAAAATAAAAATAATCAAAAGAGGGCTGAAGAGATGGCTCAGTGGTTAAGAGCACAGATTGCCCTTCCTGAGGTTCTGAGTTCAATTC
TCACCAACCACATGGTTGCTCACAACCATCAGTAACAGGATCTGATGCCCTCTTCTGGTGTGCATGAAGACAGCGACAGTGTACTCACATACAT
AAAATAAATAAAATCTTTTTTTAAAATAGTCAAAGGAATTTGAAGTCGTCCTAGGTGCCAAGGGCAAGAAAACGCGGTTGTAACACTTAGAGCA
AACTGAGATTGTCTGTGGGGAGCAAGTCAGAGCGTTCAAAACTCCACACTCGTGCCTTATGTGATTGTTAGTGGCTGTCGAGTTAAGAAAATCC
AAGATGGCAGCCTCGCGGAGCCCCAGCTGACTGCTCACGGTCTCTGCCCCTAGCTTACACGGACTTCTTCCTGCCGCTGCTGAGTCGTTGCCCA
TCTGCCATGGGGATAAAAAATAAGGATGGAGAGACTCCTGGGCAGATTCTGGGCTGGGGACCCCCCTGGGATTCTGCCGAAGAGGAAGAAGATG
AGGAGGTTTCCAAGGAGCGGGAATGGCGGCAGAAGCTACAAGGCGAGCTGGAGGATGAGTGGCAGGAGGTCATCGGGAGGTTTGAAGGTGAGGA
GCCCATTGCCACCCCCAGCCGCCCTCCCTCCCGCCCCTGCCCTGTGTCCTCTACTCCAGGCCCAATGCATGTGGCCTCTTGATTTTACTCTTTG
TCTTGTAAGTCCGCTAATCATAGCCTCTCTCCGACAACCTCATCCCACCTCCTGAACAGATGATGCCTCCCGGGACACGCAGGAGCCCGAGTCC
TTCTCAGCCTGGTCAGAGCGCCTGGCTCGGGAGCATGCCCAGAAGCAGCGGCAGCAGCTGGAGGCAGAGGGATCCTGCCGACCTCCAAGGGCTG
AGGGCTCCAGTCACAGCTGGCGACAGCACGAGGAGGAACAGCGGCTTTTCCGAGAGCGAGCCCGGGCCAAGGAGAAGGAACTGTGTGAGAGCCG
AGCCAGGAGGGCTCAAGAGGCTCAACGGGACAAAGGGCCAGAGCCTCCCAGGGCTGGGCCCAGGGCAGAACACCCACGAGGGGCAGAGAGGGGC
AGCCTCTGGCGCTTTGGCGATGTGCCCTGGCCCTGCCCTGGTGGAGGGGACCCAGAGGCCATGGCTGCAGCCTTGGTGGCCAGGGGCCCCCCTC
TGGAGGAACAGGGGGCTCTGAAGAGGTACTTGAGAGTCCAACAGGTCCGGTGGCATCCTGACCGCTTCCTGCAGCGATTCCGAAGCCAGATTGA
GACCTGGGAGCTGGGCCGAGTGATGGGAGCTGTGACAGCCCTTTCTCAGGCCCTGAACCGCCACGCAGAAGCCCTCAAGTGACCCTAGGGAATA
AGCAAGAACCTGTGGGGATGTAGCCTCAAGAATAGACGGAAGGAAGCAAGGTCGTGGATGGGGATGGACAAGAAGGGGCAGCTGCGGCACAGAG
AGGAGAGGATATGGGACGGCTAACGTAATGGTTGTGGGGGTGGGGTGGGGGAGGCTCTACCACTGCTCTGGACTGCCATTTCCTAATAAGACTT
GGTTCCACACCTCACTCCAGAGTCTCCTCTGTTTTTTCCATTGCTATGGTTTTCATCACCCACGACATCTCCTTTCCCGCCCAATTGTTGAAAC
CCACAGCTCCCACACACCTGCGGTACAGTCACAGTGCGCTCTCAATGGAGACCAGAAGCCCCTGCCCGCCCTCCTTCAGAAGGCCTGGTTCATC
GTCCAGACCCTGTCAGCTCCCCACCCCACCCCCGCCGGGGTCCTGACAAGGTTGCAGTTGAAGAAAAATGTGAACAAGAGACAGAAAAAGGAAC
AACCCCTCCCTCCATCTCCAACCACATCCTCACCCCTGCTCAGCTCAGGGCTTTCCCTCTGCTGAGTCACTGAATGAGCTGGGTTGCGCGCAGC
AGGGCTAGGGGACCACGAGGACCTGGGAGGATAGAGGATAGAACCAGAGGGGATGGAGGAAGNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNGAAGGAAGGAAGGAAGGAAGGACTGGGCGGGGAGAAACTGTGTTGTTGGCCAC
CAAGCCTGAAAACCTGAGTTCTACCCACATAGTGGGGGAAGGGAACTGATTCCTACAAACTGTCCTCTGACCTCCATAGTGCCCTGTGGCACTC
ATGCACACCACAAATAAAAGTGTAATTAAACTTTTTTAGCCAGAAAGTGGTGGCACACACCTTTAATCCCAGCACTCTGAAGCAAAGGCAGGCG
GATCTCTGAGTTCGAGGCCAGCCTGGTCTACAGATCAAGTTCCAGGACAACCAGGGCTACACAGAGAAACCCTGTCTCATGGGTGGGGAGTTGG
AGGAATAGTGGATGGTTCCTGAGAAAGAACCAAGATTGGATTCTAGCTTCTACACACACATGCACACGGACAGGTGTACGCCTGCACAGATGAA
CATACATACATTTACACACCTGCATATACACACCCGCATATACACACCTACCAAGATGACAACTACAATATCCTATAAACTTCGTACAAAGGGA
CATGAAGATGTGCCACATGGTTATTGGTAGAACTGGGGAAGTTCTTGGATCAGCACCCGTTTCAGCTGCCTAAAGGGTTGGATGTGGCCTAGAG
CAGGAAGGTTCATGGCTGAGGAGAAAGACAGGGGTTCCGTTTCAGAGAGTTTCATGAATTCAGAAAAGACACTGCTGCACAGTGAGTAAACATG
AGGCCACTGTCAGAACCCTAGGAAAGAGGCAAAAAAAAAAAAAAAATATCCAAGCTAAATCTACAGAGAACTTGGAAGCAGCCTGCCCAAGGTA
CTACAGTGCTTGGCTCAGAACAGCCATGACCCTCAGGGGAAACGTCCTGCAATCGGGTGTGGAACCTAAGAGCTTGGGCATCTCCTTCTCCTCA
GAGGTAAAGCAGCCAGAGAAGTAGGAGAATGCAGAGGCCTGGGTGGGGGTGAGGTCAGCCAGAGAAGGAGCCTCAAGTCAAGTCGTGATGGGGC
AGCTACGACTTAGGCCTCAAAGAGATCTGCTGGGCGAGAATTGAAAGGACCTCGTAGGTTCACAAACTAGACGATTACGAATTTTAAGAGAGAC
AATTTTGTTTTATTAGAAGGGAGAGTTGCTTTTCACACTGAGGAGTGAGTGGGCGGTGAGCTGAGTGAGGGGCGGTGGGTACAAAGGATGGTGG
AGACAAGACAGGCATCATCTCTACCCACGAGTCTTGATGATGGGGAAAATCTTACCAGTTCCTGTTAGTCAAGGGGCCTTGTACTGTGAAACAC
CGCACAGAGGGAAGGCGAGAGGGAGACAGATTCCAGGGAGGGAATTTAAAACTTATCTTCAAGAAATGACACCAAATACGTCCGTGCGTTAAAC
AACATACGATGGAAAAGACACTAAGGACGGGGATGAGGCGGAGGGGGAAGGATTATAGATTGAAGGATCAATTTTGAAAAGAAAAGGTTTTTCC
AAGAAAGCTGTAAAGGGGAAGCTTTAGTTAACAGAAGGGCAAAGACGGAGGCAGGGAGGGAAGCCATCTTCAGGGGCCTTAAGCTCAGTGGAGG
GTGGGGGAGGTCGAGGGTTGTGGAGAACATTGGAGAATCCCTCGGAGACTGAAACCCGGGCGAGCAGCCTGGGGGCCCACCAGCAGCCCCTCGT
GACCGATTCTGCAGCTCAGGAGGAAAACCCAGGAAATGCCTTTCATTTCCTCAAACCGAGATCTTCAAGGTCTTCTTGGTTAAACATCGGGTGG
GAACATTTTTCAAAACTGAGAGTGTAACTGAAATTGTTGAGCCTGTTTGTTTGTGGTAAACAAAAACCAGCAAGAATTGCTGGGGGCTGGCTGG
GCTGAGAAATGGCTGGGCTGGGGGATGGGACTGGGGCTGAGGCTGGGATGAGGGAGCTGGGGAGGGGCTGAGATGGCTGGGATGGGGATGGTGC
ATTTTCCACATAAGCATGGGCACTTGGGCAGCAAGCCCAGCAATCCTACCTTGAGATAGAACATGAGGAGGGAAGGAGAATCCCCAGGCACTTG
TGGGCTAGCCTGGGCTGTGCAGCCACAAAACCTGAACCTGCCTCAAACAAGGGAGAAGGTAACACAAGCCTCCCGAGGCTGCCCACTGACCTCC
AGACACACGTGTGAAAGCGCACACAAGAATTTTTAAAATCAGAAGTAGCCCGGTGTGGTGGATTATGCCTGCAGTCCAGCCTTTAGACGTGAAG
GCAAAAGCACCGGGGGTTCAAAGTCAACTTCAGCTATATATCAAAACCAAGACCAACCTATGCTATTGGACAGTCTCAAGTCCCACATCCCCAA
AAATAAACACCAAGAAAGAATTCAGGGGGTTGGAAAGATGGCTCAGTCATTAAGAGTGCTGACTCTTCTTCCAAGGATTCTAAGTTCAAATCCC
AGCAACCACATGGTGGCTCACAACCATCTTCTGGTGTGTCTGAAGACAGTGACAGTGTAGTCATATATGTAAAATAAATAAATATTTAAAGGAA
GGAAGGAAGGAAGGAAGGAAGGAAGGAAGGAAGGAAGGAAGGAAGGAAGAGGGAGCGAGCTGCAGAGCTGGCTCAGAGGTTAAGAGCACTGATG
CTCTTCCAGAGGTCCTGAGTTCAATTCCCAGCAACCACATGGTGGTTGACAACTATCTGTAATGGGATCTGATGCTCTCTTCTGGCATGTAGGC
ATACCTACAGGTAGAACACTCATATACATAAAAAGTAAAATAAAACCAAAGAAAGAAAGAAAGGAAGGAAGGAAGGGAGGGAGGGAGGGAGGGA
GGGAGGNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNACCCTATCTTGAATAGAAAAAAACATTGAGATAGGTCTTACTCTGTAGCCTTG
TCTATGTCTAGTCTGAGCTTCCTATGTAGGCCAGGTTTCTCTCAAACTCATAGAGATCCACCTGTCTCTCCCTCCCCAGGTCTGGGATTAATGG
CGTACACCACCACACCTGGCACCAGTGATGTGAAACTGACCTTTGAACCCCACAAGGTAGAAAGAGAAACTGAACTGATTCCCGTAAGTTGTTC
TCTAACCTGCACACATGAACCGGGGCACGTATTCACCCCTGCATACACAAAATAATTTTTAAAATGTAATTTTTTTTAATGAAAGAGAGGCTAG
AGATCTGAGTCAAAGTGAAGGATTAACTCAGTGGAGAAGAGAGGATAGAAGAAACAGGTTATTATTCCTAGGGAAAATTCAGAGATCAAAGTCT
TAAAAAAAAAAAAGAATCATTTTATCATTAATCTGTGTGTGTGTGTGTGAGTTGTGTGTTGTGTGGTTGTCCCCACAGAGACCAAAAAAAAAGG
ATCAGAGCCCTTGGGGCTGGAGTTCTGCCGCACCTGACATGAGTATTGGAAACTGAATTCCTCCTGTCCTCTGCAGAGGCAGTACATGCTCCTA
ACCAGTGAGCAGTCTCTCCAGCTCCCAGGGTTGAAGCTCTTGAAAGGCAGCCTTGCTTCCAGGGGAAGCTGAGGTGGGAAGTGTGGCCTTTCCC
AGAGAGAAGCTGAAGCTGGCCACCTTAAGGCTGGAGAGCTGAGCCCAGAGCCGAGGACAGACATGTGCACTCTGTGTGGGTTTGAAGATGGTGC
CAGGTGATGAAAGATGAAGAGAAACTGAACCAGATGTCCAAGTTGTTAGGCATACAGTACAGATGTTGATGGAGATACAACTGTGAAGACAGGA
CGAGTACGGTTCGTGCAGAGTCAGGAGCCTCTAAAGAAGAGAATCCTCTTGGTAGCATGGCCACACTCCTCCGTCACTTCTCCCTAATGCTGGT
GTCAAAGGAAGAGCTACCTTCTCCAGAGAGGGCACAGGCGTTGTCTGGTGCCTAAAGAAAACATCTTCAAAGATGAAACAGGGGATGTGCGTAG
ATATTCCACAGCAAGAGGACACTGTGAACCAGATTCACAGAAGAGTCCTTGCTGGGTAGTGAACCACAGAGGGGACCAAGGAGAAGGACATCCT
GACAGGCTCGCTTTTAGAATCCATAACAAACAGGCTCGCTTTTGGAATCCATGACAAACTATGCATTCTATCAACACCTGGCATTTATTGTGGG
GAGGCGTGGGACACTAACAGGCCCCGTGGTCCAGGGCTTTTACATCCCAGCCCACCATTATGCTGGCAGCTCCTTGTTTCCCTCTAACCCCATC
TTGCCTGTGAACCTGAATGGCTTCAGACTTAACAACTCTCTCTCTCTCTCTCTCTCTCTCTCTCTCTCTTTCTCTCTCTCTCTCTTAATAAGAT
TTATTTATTTATTTTATGTATATGAGTACACTGTAGCTGTACAGATGGTTGTGAGCCATCATCTGGTTGCTGGGAATTGAACTCAGGACTTCTG
CTCACTTTGGCCCCACTCACTCAGGCCCGCTTGCTCTGGCCCAAAGATTTATTTAATATTATATGTAAGTACAGTGTAGCTGTCTTTAGACACA
CCAGAAGAGGGCGTCAGATCTCATTACGAATGGTTGTGAGCCACCATGTGGGTGCTGGGATTTGAACTCTGGACCTTCGGAAGAGCAGTCAGTG
CTCTTTCCTGCTGAGCCATCTCTCCAGCCCCAGACTTAACAACTCTTGAGAGTGGATTTAGATCTCATGTACCCAAAGCAGCACATTTGGCTTT
TCCTGCCCACCTACCTAAGGACCCTACCTTAGCTCCCATCCAGCTCCATCACCCAGACCCCTGTTTCTCCTCTTGTTCTTCTGCAAGGCAGCAT
AGACCAGGGAGCCACCTTGTAGACCAGGCTGGCTTCAGACTCACAAAGATCCACCTGCCTCCCAATCACCACCATCCCTCCTCCACCCTCTTCT
ATCACACACACTCACACCTTCCAAAAGGTCCCACTTGAACCGGAAAGATCACAGTTGAATAAAATGTCGATACTTTGTGTCTTCCAAAAGATCA
CAGTTGAATAAAATGGAGATACTTTGTGTCTTCTCACATGGTCTGGCCCTGCCTTCCTCTCCCACTGCATCTGACCTCTTCAAGTGTCTCTGAT
GTGGTCAAGTTCAGTTTCTTGCTGTCCATCAGCATCCCTGGCTTCCTCCCTCCTCCCTACCTATCTGTCACCTGGGCCTTTTCTTCAGTGCTAC
CCTTGCTGAGAGTCACATTACTGAGTGCTGTATCCAAGAGTATTCTCTTCCACTCCCCACGCCACCCTGCCTTGTTATCCCTGTAGGACTGTTT
TACAGATTCATTTTATTTTATGGCGATGCTGGGGATGGTCCCTGGGTCTCACTCACGCTAGGCAAGTGCTCTATCTCTAAGACACATCCCTGCT
CTTTGATGTGTCTTTTCCATCTGGCCTGGCTGGAACATGATCCCTAAGAGTAAAGGATTTACTCTTAGGATTCTTTGGCTCAACTTTGTGCCAC
AGTATACTTCCTGCCTTGAACAAACTTCCTGGTTGGTACTCAGTAAACATGTATTAAAAGCTGTTGGAGGGTGCGGTGAGGTAGGACAATGTCC
AGCTCTAATTTCTGGCTCAGGTAGAAGTTCCATCTAGTATCATGCTTTGTTTGGGGTCAGTAGCTGGATAACACCAGAGACAGGGTAAGCTGTT
TCACAGGAAGGAGTTTATTTGAGAGTCCTTTGAGGGGTATGTGCTTAGATTTGAGGTTACGTTTGAATTGTGATTTGGGTGAAGGTTAAACTTG
GGTAAAATCTGAGGCTAAAAAAACAGAGACTCACAGTAGTCTTTGACTACTGTCAGTTCAGCCTGGTTTGCAGGCTCCGGAGCTTTCTGTGGCC
CTTCCTGAGAGTGGTTATAGACGATGCACAAGACTCCTAGTCATCAGCCCTCCCCCCCGGGGAAAGTCCCAGACAGCTGTTGGCCTCCCCAAGA
GAGAAACTGAATTTCACACAACCTCTAAAACTGAGATTGAAACCAAAATTGGCCCATGGCAAGGCATATCCGCCAGCGTGCTGCCAGCTCCCGT
GACACAGCTTAGCCAAGGCTTACCCTTCCTTCGTCTGTCTCCATCATCCCCTTATGCACCCTCTCCTTTCCTCTGCCCCTCCAGGTCCCAGCCT
TCTGCTCTGACTCAAATCTAATCCTCTCTCTGTCTCTCTTTCTGTCTCTCTCTGTCTCTGTCTCTGTCTTTCTCTGTCTCCGTCTCTCTGTCTC
TCTCTCCCTCTCTCTCTGCCAATTTTCATTTTAATACAGCTTAGCCAGCGTCTCGGCTACCATCAGTTAAGAGACAAAAGCAACAGGAAGCATA
AAGACTCCCAGAGAAAGAGGAACAAAGAAAAGGAGCCAAAGATGACACAATGACAGAATTAAATTCTGTCTGAGAGAACTCAGTGAAGAGAGCC
TGCTTAGCCTGCTCAGGCTGTGGGTCCCATGGCCAGTTCTGCCAAA
MOUSE SEQUENCE—mRNA
CCGGGTGCATCCTGGCGCGCCTCCTCCCTCCCCACCTCCCTTCTCCGAGCGTCTTAAGCCCAGGCCTCCGGCCTCCGTCTCTGAGGGTCCTTGG
GGGGGGCGGGGAGCAGGTCTGATGAGTAACCCCTCCCCCCAGGCCCCAGAGGAAGAAGCCTCTACGTCTGTCTGTCGGCCCCAGAGTTGTTCCA
TGGCCTCCGCTTCCCGCCGGCACCGCCGAGAGCGACGCTTCCGGCGGTACTTGTCTGCAGGACGGCTGGTGCGAGCCCAGGCTCTCCTCCAGCG
ACATCCCGGGCTTGATGTGGACGCTGGGCAGCCCCCGCCTCTCCACAGGGCCTGCGCCCGCCACGATGCCCCTGCCCTGTGCCTGCTGCTGCGT
CTCGGCGCTGACCCTGCCCACCAGGACCGCCATGGCGACACGGCGCTGCACGCTGCTGCCCGCCAGGGCCCTGATGCTTACACGGACTTCTTCC
TGCCGCTGCTGAGTCGTTGCCCATCTGCCATGGGGATAAAAAATAAGGATGGAGAGACTCCTGGGCAGATTCTGGGCTGGGGACCCCCCTGGGA
TTCTGCCGAAGAGGAAGAAGATGAGGAGGTTTCCAAGGAGCGGGAATGGCGGCAGAAGCTACAAGGCGAGCTGGAGGATGAGTGGCAGGAGGTC
ATCGGGAGGTTTGAAGATGATGCCTCCCGGGACACGCAGGAGCCCGAGTCCTTCTCAGCCTGGTCAGAGCGCCTGGCTCGGGAGCATGCCCAGA
AGCAGCGGCAGCAGCTGGAGGCAGAGGGATCCTGCCGACCTCCAAGGGCTGAGGGCTCCAGTCACAGCTGGCGACAGCACGAGGAGGAACAGCG
GCTTTTCCGAGAGCGAGCCCGGGCCAAGGAGAAGGAACTGTGTGAGAGCCGAGCCAGGAGGGCTCAAGAGGCTCAACGGGACAAAGGGCCAGAG
CCTCCCAGGGCTGGCCCCAGGGCAGAACACCCACGAGGGGCAGAGAGGCGCAGCCTCTGGCGCTTTGGCGATGTGCCCTGGCCCTGCCCTGGTG
GAGGGGACCCAGAGGCCATGGCTGCAGCCTTGGTGGCCAGGGGCCCCCCTCTGGAGGAACAGGGGGCTCTGAAGAGGTACTTGAGAGTCCAACA
GGTCCGGTGGCATCCTGACCGCTTCCTGCAGCGATTCCGAAGCCAGATTGAGACCTGGGAGCTGGGCCGAGTGATGGGAGCTGTGACAGCCCTT
TCTCAGGCCCTGAACCGCCACGCAGAAGCCCTCAAGTGACCCTAGGGAATAAGCAAGAACCTGTGGGGATGTAGCCTCAAGAATAGACGGAAGG
AAGCAAGGTCGTGGATGGGGATGGACAAGAAGGGGCAGCTGCGGCACAGAGAGGAGAGGATATGGGACGGCTAACGTAATGGTTGTGGGGGTGG
GGTGGGGGAGGCTCTACCACTGCTCTGGACTGCCATTTCCTAATAAGACTTGGTTCCACACCTCAAAAAAAAAAAAAAAAAAAAAAA
MOUSE SEQUENCE—CODING
ATGAGTAACCCCTCCCCCCAGGCCCCAGAGGAAGAAGCCTCTACGTCTGTCTGTCGGCCCCAGAGTTGTTCCATGGCCTCCGCTTCCCGCCGGC
ACCGCCGAGAGCGACGCTTCCGGCGGTACTTGTCTGCAGGACGGCTGGTGCGAGCCCAGGCTCTCCTCCAGCGACATCCCGGGCTTGATGTGGA
CGCTGGGCAGCCCCCGCCTCTCCACAGGGCCTGCGCCCGCCACGATGCCCCTGCCCTCTGCCTGCTGCTGCGTCTCGGCGCTGACCCTGCCCAC
CAGGACCGCCATGGCGACACGGCGCTGCACGCTGCTGCCCGCCAGGGCCCTGATGCTTACACGGACTTCTTCCTGCCGCTGCTGAGTCGTTGCC
CATCTGCCATGGGGATAAAAAATAAGGATGGAGAGACTCCTGGGCAGATTCTGGGCTGGGGACCCCCCTGGGATTCTGCCGAAGAGGAAGAAGA
TGAGGAGGTTTCCAAGGAGCGGGAATGGCGGCAGAAGCTACAAGGCGAGCTGGAGGATGAGTGGCAGGAGGTCATCGGGAGGTTTGAAGATGAT
GCCTCCCGGGACACGCAGGAGCCCGAGTCCTTCTCAGCCTGGTCAGAGCGCCTGGCTCGGGAGCATGCCCAGAAGCAGCGGCAGCAGCTGGAGG
CAGAGGGATCCTGCCGACCTCCAAGGGCTGAGGGCTCCAGTCACAGCTGGCGACAGCACGAGGAGGAACAGCGGCTTTTCCGAGAGCGAGCCCG
GGCCAAGGAGAAGGAACTGTGTGAGAGCCGAGCCAGGAGGGCTCAAGAGGCTCAACGGGACAAAGGGCCAGAGCCTCCCAGGGCTGGGCCCAGG
GCAGAACACCCACGAGGGGCAGAGAGGGGCAGCCTCTGGCGCTTTGGCGATGTGCCCTGGCCCTGCCCTGGTGGAGGGGACCCAGAGGCCATGG
CTGCAGCCTTGGTGGCCAGGGGCCCCCCTCTGGAGGAACAGGGGGCTCTGAAGAGGTACTTGAGAGTCCAACAGGTCCGGTGGCATCCTGACCG
CTTCCTGCAGCGATTCCGAAGCCAGATTGAGACCTGGGAGCTGGGCCGAGTGATGGGAGCTGTGACAGCCCTTTCTCAGGCCCTGAACCGCCAC
GCAGAAGCCCTCAAGTGA
HUMAN SEQUENCE—GENOMIC
TAGTTGTTTGGGGATCTGGGCCTTGGTACAGTATCAAATAACACCAGAAACTACTTTCTGGTTTCAAGGGGGAAAAGAACAACTGTGGAATCAG
ACTGTCACGACGCTAATCCTATGGTAAATCTAAAATCATTAATGAGGCCAGGTGCAGTGGCTCACTCCTGTAATCCCAGCACTTTGGGAGGCCG
AGGTGGGTGGATCACTTGAGGTCAGGAGTTCGAGACCAGCCTGGCCAACATGGCGAAACCCTGTCACTACTAAAAAAAAACAAAAATTAGCCAG
GCATGATGGCACACTGTAGTCCCAGCTACTCGGGGGGTTGAGGCGGGAGAATCGCTTGAACGTGGGAGGCGCAGGTTGCAGTGAGCTGAGATCG
CGCCACTACACTCACAGCCTGAAGGACACAGCGAGACTCCATCTCAAAACAAATAAATAAAAATAAAATAAAATAACTAACATAAGTCGACCAG
ATTTGTGGCATAACAGGAGATACAGCATCACCTATGAAGGATTCTTGCCAAAAATGCTTAACTTCAATCAGATTTTTTCTTTTTTTTTGAGATG
GGAGTCTCACTCTGCCACCCAGGCTGGAGTGTAATGGCACAATCTCAGCTCACTACAACCTCTGCTTCCTGGGTTCAAGCGATTCCCCTGCCTC
AGCCTCCCAAGCAGGTGGGACTATAGGTGTGTGCCACCATGCACGCCTAATTTTTGCATTTTTAGTAGAGACAGGGTTTCATCCTGTTGGCCAC
ATTGGTCTTAAACTCCTGACCTCAAATAATCCACACGCCTTGGCCTCCCAAACTGCTGAGATTACAGGTGTAAGCCATTGTGCACTTGGCCAGA
ATCCTCAATATTCACACACCACTGGAGCTGTTTTAAAGTTTCCGGCTTTCTCTGCCACATACCCCAAAATTATTAAACTGATATGATTCAAAGT
CAGTATAAAGTAGTAAGAAAAGGGTGGTCTTGTGTTAAGCATCATCCATAGCCCAATTACGAATCCTCCTGTTACATAGGAACTCAACACTCTG
TTACACCACAGCAAACTAAAGCTTCTCCAAAATTAAAGAGACTATTGGCCTACAAGTTTCTTATCCCTCCAACTTGCCACACCCTCACTCTCAG
GTCTCTTTACCTTGGCTTACCTTGACATTGGGCATGTATTTAGAGAAGCGCTCATATTCCTTGCTGATCTGAAAAGCCAACTCCCGAGTGTGAC
ACATCACCAGCACAGACACCTTAGGCAGGAAGTATACGGAGACATATGGTAAATGTAGCTCTTCATTATCCCCTCTAGGGAAGTGACTGTCACA
AAAACACACCTGGGCCGATAATAAATGACTTCAATTCTGTGATCTAAATCATGAACCCCACGCTTGCGACAGAACATCCCCCACAGCTGTCAGG
TTGTCAAGGGTAACAGAGGTCATGTGCTCATGGCTCTGCAAGCATCATGTAGTTAGGACAAAAACACCCTTCCCTTATAGTCCTAACCAAAATC
CCCTCCCCAGCACTCTCCCCAAATATACCTGCCCAGTAACTGGCTCCAGCTGTTGCAGTGTGGCCAAGACAAACACTGCTGTCTTTCCCATGCC
CGACTTGGCCTCCCACAGGACATCCATTCCCAGAATGGCCTGAGGGATGCACTCATGCTCGACTAAAAGTTGCCGCGGGAGGAAGATAAATTAG
ACTTCAGTCTCCAGATAACTCTACCTTTTTCACCATGCCAAGCCCATTTCTTACCACTCAATTCTCAAAGTCTAGTATTTACCTGGTTCTTGCC
AACTTCCAGACCCATTTTACCTCTCTCTCCTCAATTACATTCACCTCAAAATCAGACTCTCCTAATTCCTCCTAGCTTTAGCCTCCTCCAGATC
TAGGCCTTCCCAGTCCTAGTAACAAACCCCTTGCATCTACCAACCGCTCACCTTCAATGATCCTAGCTCTGTCCTTATTTTTCTTAATCTGTAA
CAATTCATGACATTTGAATACCTGCCACAGACCACTTCTCCTGCTTAGGTTGCTATACTTCGGGTCACGTAACTACTACAACCCTGGACAAAAT
GAAGGACTTGGTACCTGACCCAGAAGCCAGTCATCTCTAAACCAGTCATAGAGGTTTCCAGAGACCACAGTTGGCCTGGCCCAACAGAGGGAGA
CTACAGGTCCAAGCAGGACCTTTCTGGAAATTTAAAATTAGAAGTCAAGTGACAAAATTAAAAATAAGCAGACAAGAAAAGCCAGTCACAAGAA
TGAATGGCAGACCTGGAAGTCACTTTTGGATCATTAGCACTTTGGTGCTATCACGAAAGAAAGAATAAGCCTGTATAAGCCTCCTCTATCCAAA
ATTGTTTTTGATACTTATCCCGATTTTTTCTTCCTCACTGTCGCCCCGGCTGGTGCACAGTGGTGCAATCACGGCTCACTGAAGCCTCAACCTT
CACCTGCTTAATTTCTGAACGTTTTGTAGAGACAGGAGTCTCGCTATGTTGCCCAGCCTTCTCTTGCACTTTTGAGCTCAAGTGGCCACCCTCC
TGCCTCGGCCTCCCAAAGTCCTGGGATTACAGGCGTGAGCCACTGCACCTGGCCCTCATCTAGCCTTAAGTATAAACCCTTACCACCACCTGAG
CAACGACAAACACATCTTTGTATTGTACCCTTAAAGAGCCCAATGAGCACTACATGCCCAAGAGAAAATTTACCTTCTGACGGATGCTCAAAGC
CACAGTCGACAATGGCCCGGAGCAACTCTGGCTTGAGCAGGAAGTCACGAAAGCCAGAGCTGTGGATGGAGACATAGGAGCCCTTGACATCCTT
CTTGGCAGGGGCCTCAGCCCCATCTCCCCCAGCTGCTCTCTCCACCTCATCATCTTCATAGTCCAAGAGCTCATTCTCCACATCGTTCTCTGCC
ATAACTGGGCCGGCAGGGGAAGAAGGGAAGGGGGATCTGGATGGGTTCTCGCAAAATAGGTGAAAACAAGGGGTGAAGAGTAGGGGATTGAGGA
ACAGCAAAGGAAAACAAAGATACTATTTCTAACAGAAGAGCTGGAGGGGGGAAAAAAAAAGCAAGACTTAATCACGAGCACAGCCTTCACCATT
TTTCCATCCCCAGTTCCCACTTTCCCTAAACCAGGAAACTTTTACCTGGAAAGAAAAACAGATACAAAACATAAAAACGAAAAGCAAATATAAC
AGAACAGAAAAAGCAGTACCAGGGAAAGTGGTTAGGACAGAGGTTCCCAACAAGATTAGCAATCACAGTAGCGGAAACCAGAAAAGTTGGAAGG
GGAAGACCAACTTATAAATTCTTGATCTGAAAGTAACAGTGAGGAAATAGAATAGATAATAAAAGGTAAAATATGACTAATAACTTAGTAAAGT
GGAAAATGGAGATGACAAGTAGAGTCCTGAAAAGTCCTCAAAGGAAGACTCCGCTTTCCCTATTATAATCCCACCCTTATGGATGCCTAACTCA
GCAGCCATCAGTCAAGGGTGATAGATGAGGGTCATCACTGCGCAAAGCGCTCACCTTTCGAAAAGAAAACATCATATGGCCGCCGTCCACCTCC
CATAGCTCTCAGCCTCCCACTTCTCAGTATCCTCCCTTCCGCTGTTTAAGCAAGCCTTGTGTAATTAGCATGGGGGGGAGGGGCGGTGCAAGAC
AAATGGCTCGGCCACAAAAAAACAAAATTCATGTCTCCACCCTACAATAAGAAAGCTAATAGGTGACAGAGAAAGGCAATCCCCGCCCAGGCTT
TAACAGGATCTTTACCAAGTGGTCTCACATCACTGTTACGCTACGAAGGTGAGACTCCTTTTGGAGAAACATACAATGACACCAATCGTATCGT
AAACACTTGGAAGGCACTCCAAATTAAGTTGGGCAAGTCAAGGTGAGAAAAATCCAACTGGGCCCAGAAACCAGCTCCTCCTCCCAGTCCCACC
GAGGGCCGAAAAAGAGCTCAAGAAAGAACAAGGAAGGTGAGAAGAGCCCCGCCCTCCGCAAATACCAAGACCAAGGGACGCCGAGCACCGCCTC
TCATTGATGCTGAGGCCTCCAATATGAGAAGAACCCATTGGAAGAAGGGAGCAAAACGAACACAATGGCGCCGAGGACACCATCTTGGATTGGG
TCCCCCCTTAGCTTCCCTTCCTTCCCCCAGGAGCTCTTTGCTCTCGAAAGGGATGCAAGCTAAGGAAATAGCGAACCAACTAGGCCCCAGCGAC
CAGACCATCGCCTGTGAAAAGGGTATCAGGAACCCATGTGACGGGATGGGTGCGGAGAAGCGCAGATGGAAACGGATTGTAGCGAAGGCCAAAG
CTTACCTAAACAGGGAGAGCGCCTATGGCGGCAGCAACAGCGACGAAGGAGGGAAATCTGCCTTCACTTCCGGTTCCAGGCTTCCCTCTACTCC
AGCCTCCCGCCTTCTTGGCTGCAAGAGCGCAGGCGCAAGGGACCGGAAACAGGGCCTTCCGCGGTTATACAGATCCGTGCGCTCCAGGCTTGCC
TTTGGAAAATGCCTGTCTGAAATTTGTTTTAAAACCGTTTCTAACTTCACTGCTACCTCCAGTAACAAAAGATATAAAGGAAACTAACGTCTCC
CCCCCACTGTTATCTTTATTCTCTTATCCTACTCCTCTCCATGCCCCTCATCTCTTCGTTTAGGTTTTTGCCACGCAGGTCTTCTCTGTAGGCA
CCCCTCCGTGGATGCGCGAGGAACGAGTGTGGCGAAGGCTGCGAGTTCCCACGGGGTCCTTGGCCCGGTAGTGAAGGTGACCTGAGGACTGCTG
GGCACGCACTAGGAACCGGCAGGCCCTAGCTGAGGGGAGGGAGGAGGGAAGTCTCAGGGAACTGGATTGCTCGGGGGTGTTTCCCGACTCTTTC
CCAGTCGTGGGGCTGGTGGGCGGTATTTTCCCAAAAGGATGCTGTCCGAGGTAGCTGATGCCCTAGGGCCAGTGAGTCAGGAAGGTGTTCTGAA
TCCGAGCGGGAAGACGGGGTCTGGATTCGGCCCCAAGTGTTAATAGTAGGGCTTGAGGGTTATACTACATTCCATTAATACTGTTTTTGTTTTT
GTTTTGACACAGAGTCTCGCCCTGTCGCCCAGGCGGGAGTGCAATGTCCTGATCTCGGCTCACTGCAACCGCTGCTTCCCGGGTTCAAGCGATT
CTCCTGCCTCAGCCTCCCGAGTAGCTAGGATTACAGGCGCCCGCCACCACGCCCAGCAAATTTTTGTTTTTTTAGTAGAGACGGGGCTTCACCC
ATGTATGACCTCAGGTGATCCACCCACTTCGGCCTCCCAGAGTGCTGGGATTACAGGCGTGAGCCACCGCGCCCGGCCCATTAATACTGTTAAT
TCGAGCAGAATGTTCTTGGCCCCGCCCCAACAGCCCCATTGTTCAACCTGGATTTTTTTCCTGAATGAAACATTTGCTATCCCCGTCTTTGAGA
TGGGGAGCCACAAAAGTAAGACCTGATGTCCTGCTGTGTAATAAAACAACAAACGTTTGGCCCTCTCCCTGTTAACATACTTAATCATTTAATA
CTAAGGAGTAGGTACCGTTATTCTCATCTTATTGACAGAAGCGAAGCAAAGCAACATATCTCAAGCAGTACGGCTGGTGAGGTTACAGCCAGGA
TGCAAACATCTCTCATTCTCTATTGTATTCTGCCTCCCTGCTCAAAGAATCTGGTTAGTAAATACACTGCAGGTTACCTTATTGGTTCAAATTC
TTGGTGAAGTAAGCTTGTCTTCAGTGACAAATGAAGTAACTAATTCAAGAATGGTGTCATAGAAGGTATTTTCCCAAGTATCATTTAATTTATT
CAAAAGTATTTATCAACTGCCTCCCTTGTGCCACATGTTGTCCTAGGATCTGGGGACACAACGGTGAACAGCCCTGTTCTCACAGTGTTTACAT
TACAGGAAAGAAAACACATAAACACAAATATAATGTCAAGTATCGATAAGTGGTCAGGGTGCAGTGGCTCAGGCCTGTAACCCAACCCTTGAGG
AAGCCGAGCCCGAAGGATTGCTTGAGCCCAGGAGTTTCAGACCAGCCTGGGCAAGTGACACCCCATCTCTACAAAAAATTTTAAAATTAGCAAG
GCATAGTGGCACTCGCCCGTAATCCCAGCTACTCAGGAGGCTGAAGTGGGAGGATCATTTGAGTCCAGGGGGTCAAGGCTGCCGTGAGCTGGAA
CTCCAGCCTGGGCAACACAGCAGGACCTTGTCTCAAAAAACCAGTAGCAGTAAGTGCTATGAAGAAAATGCAAGGTAAAGGGGCAAAGAGCACT
TGCTCCTACACTCCAGCTTTTCTCTACAGTTGCGATCTATAGTCCTCAGATTCCCAAATGAGGAACCATGTTTCTCACTTTAGAGAAATAATAA
AGTACTACTTGTTCTTGTTTCTCCAAGAAGTTTCAAAGGATAGCCATTTGGGCTGTTTAGGGAATATGTAAACAAAAAACAAGAAAGTGACTGA
AGGCCAGGCACAGTGGCTCACACCTCTAATCTCAGCACTTTGGGAGGCCAAGGCAGGTGGATCACTTGAGGTCAGGAGTTTGAGACCAGCCTGA
CCAACATGGCGAAACCCCATCTCTACTAAAAATACAAAAAATAGCCAGGCGTGGTGGCACACACCCATAATTCCAGCAACTTGGGAGGCTGAGG
CAGGAGAATCGCTTGAACCTGGGAGGCAGAGGTTGCAATGAGCTGAGATCACGCCATTGTATTCCAGCCTGGGCAACAAGAGCAAAACTCCATC
TCAAAAAAAAAAAAAACAAAGTGACTGAAAATGAGAAATGATGAGGCAAAAGGAGGCTGCTTCAACTCACCAATTTATTTGCCAATAATTATTT
TATTGATACTTTTTTTATTGTTACAATGGGAAAGTAAGGTGTCAAGGATATAGAAAGGAAGGGCATGCATATGAGGGAACACAGTATCATTTTA
GATCTTAGAAAGCAATGAGCATCTGATAAGTCTTTGGGGAAATAGGAAAGGAGGAAAATCTAATAAAGACAAAGATCAGCAAAAGAAAAACAAA
GAGAGGCTACAAAATGCAGTTATCTACCTGGAATTATAAGAGAGGGGCTAAATGTAGTCATCTCCTCTTTTTGGAGATCAGAAGGTCTCTGGGA
AAAGAGAAGAACCAATTTTTCAGAAAATAACTAGGGTCACAGAATGAACAAGTGGAATTAGAGAGCCAGTGATGGACGTGAGGAAACAGCTGTG
TAGGTTTTGACCAGTGAGCAGGTGGTGGTAATAGTATCACAGGGTTGCTACTTACTGAATCACTGCTACAACATGCAAGGAACTGTGCTAGACT
TTACAGAATGATTCCTAATCATTGAAGCAACCCTCACAAGGTAGGCATTATTATCATCCCAGTTTCACAGACGAGGACATCGACGCTACCAAGT
TAAGTAGCTTGTCCTGGTTTCACAGCCAGCAAGTGACAGGGTCAAGAGAGGGACCCACATCGGCCAGACACTGAAGTCAGGATGTTTTCCACAT
TCCTACTTCCCCATATTACAAATTTCACAGAGGGTTTAGGTGAGAATGACTTGGAAGTTTACAAAGTCCCAGTGAGGGTTAAAGAACAACAAGG
AGATTCAGATGTGAGCAGGATATTTATAAGTGTCACAGGAAAATTATTGGATCCTGCCTCCCAGGATTTCTAGGGGATGGAAAGAAGACAGGGA
TTATGGTGGGAGGTGATTTTGATTGGAGGATTTCTTTGAGGGAGGGAACTGGCAGAAGGAGTCAGGCCCTACGGTGGCCCTAGGCAGAAATCCG
GTAGTTGGGGTGGACCTGGGGCCTGACGTCGCAGACCATGCCAAGAAGCTGGGCCAGGACACGCTCTCGGTTTCTCTGCTGGGAGCTCTGCATG
CCCTGCACCTGGCGCCTTGTAGCCTGCTCCACCTCAGCAGACAGGTTCCCCTGGGAGCCCATGGCCTGGGGGGTAGGGAGAGGGGTGGAAGAGA
GAAAGGGAAAAGCAGAAACAGACAAGGGTCCAGGCATATGAGGGGAAAGATCCTGAAACAAAGCCTAGAAGAAAGGCCCTCTCAGAAACCACCC
CCATCCCACAGAAATATCCCAACACCAAAGAGATCAACACAGTCCCCTTTCCCCTTAGACCTAACATGCAACTTCATCCTAAAACAGACCGTAA
TATCCCCACCACCTCACCATCCATGACCATAAAACTCTACCCTCCACCACAAATGTTAATCATACTCCACATAGATGTTATACTTTACACAGAC
TGTGGCATTCCGCCCACAAGCTCTATGTGGCCTTCAAAACTCCCAGACTCTCCTACATATCATCACAAAGTTTCACCAATGTTGTGGTCCCTGC
CAGGGTCCCCTCAGCCTCAGCCCTCTGCCACCATATTTTCTTGTTGAGTCACCCTTACACACCTCACTAGATGCACCCACCAACTTGCAGTGGG
GTCTCATCCCGACTCTGCCTCAACTCACCGCCTGCTGCTTGCTCTGGAATTCGTGCTCTCGCTCTCTGCGGTATTGCTCCACCTCCATCTGTGC
CTCCTCCTTTGCCTGCTTCAGTCGCCGGGCCTTCCCTGGAGGCAGAAGAAAGGACAGTGAGTGGGGATGGACCCACACACACACAATGTAATAG
CAGGAGTCAGTCCCTTCCAGAAAGTTATACAGCCTTCTCTCAGCCAACCAGGTGCCAGATTCTAATATCCATCCATTTCTTCCCTCCTAACCAG
CCTCCAGACCCTAGCTGTCTTCCCGCCAGCCTTGGGTTTTCCCAAAATGTTTGCTGTCCCCCACCCCCAATTTTCTTTCCAAACTCCTAAGGGA
GGAAAGAGGAGACTCACTCTTTCTGGCATCTGCCACCTTCTCAGCTGCCCGCTTCTCAGCTTGCAGAAGCTGCTGGATACCTTGGGACTGACTG
GCCATTTCTGTTGTTATGGCCGATGCTGTTTTGAATGCTGTCAAAGTACCAGATGGCTCCCACCCCCCACCGCTTACTTCTCCTCCTCCAGCTC
GTTGCTGCAGTCCTCCACTACCCCTGGGTCTTAGTGCTCCCCTGCTCACTCAGCCTCCTGCACCGAGTGTCTCTCCCAATCTCATCCTCCTATT
GATGACTGGTCCTCCTCTCCAGCACTTCTTGCTCAGGCAGTACCCAAAGGGGCCGCCTGGGAGCAGCAGAGACCAGGCCCAAAGCTGCGGGCTT
ACAACAGGTTAGCCATCCCAGTCGGAAAGGTCTAGGGATGAGGCAGGGGCGGAGACGGGGGAGTACTGAGGTGAGAGAAGGAGAACTTGATTGG
TGGTAACAGAGGAAGCATAAAGGGTTGTGAATGCGGTGAAAAGGTAAGGATGTCATCATGCAACCTGTGTTGGGAAAAGAGCATTCTGGGCTTA
ATTCTAAACTAACTCTCTACCTTTCTCTCTCTCTCCACCATCCCGCCCCCTCCCCTGCCTCCCGTTGTTAACATCTCCATCTTTTTCTACATAT
TTCTCAAGTCCAAATTTTTGCATCTCACTTGCCCCATCCTACGATAGTCTTCTTCCGTCTTTTGTCTGTATTTTTTCTTTTTTTTGATCTGTCC
CTGTTGTTGTCCCACTGTGGTTTTTGTTTTTGTTTTCCATGTTTAATGTGATTTTTATCCTGTCTTTATCTCCTCTATTTTCTCTGTCTTCTCA
TCTTTTCGTCCATCACTGAACCATCTCCTCTCTCTGCCAAGTTAGAGGAGGCGGGGAAAAACCTCCAAATAACTCTCTTTTCTCCCTCCCCTCC
CCTCGCCTCCTTTTCCTCGCCTCCAGTCCAGTCTTCTGGTTTCAGACGGCCCCTTTAATTTAAGTTCCCTAGTTTCCCCTGGGAGATCTGGCCA
AGAACTACCCGGTCGGGGCGGAACGACATCCGGTAACGCCCCTCACAGTTCACTTCCGTCCTCCACCTGCGTCTCTGCTTGCGCCATTTCCTCC
AGCCTGGAGTGTCTCCGCCCTTCCCGCCTCCCGTCTCCGAGCTTCTTAAACACAGGCCTTCGGCCTACGGCTCTGGGGGTACTTGGGGGGGCGG
GGGCAGGTCTGATGAGTAACCCCTCCCCCCAGGTTCCAGAGGAAGAAGCCTCCACATCTGTCTGCCGGGTACATGATATTCAATTTCTAGATCA
TTATTGGAGATTATCTGTGACTTTTTAAAACTCAGATTTCTGCTGATAAAAATTTTCCCCATCCGGCCCTGTTGGGTTTTTTTAAAGTTCTTTG
TTAAAAATTAAAAATTTACCTGGGCTCCTGAGCCTTAAACCAATTATTTACCCTTTTCTCGAATTTTACATTAAAAAAATTAAACCTCTGATCC
TATCACCCCCCTCAAAAAAAATTTTTTTTCAAATCTATCATCTGATAAAGGATCAGGGTTAGGTTAGGCCTCATCTCTTGCTGAAGATATTAAA
AAAAGACGGAACCAAAGGGAGAAACAACAGGGGATGTCAGAGATGGAGGGAGAAGGACCAGCCAAGGCTGAAGTCCTGACTGCTGCCTTTTTTC
CTTCCCCAGCCCAAGAGTTCCATGGCCTCCACTTCCCGCCGCCAACGCCGAGAACGTCGCTTTCGTCGTTACTTGTCTGCAGGACGGCTGGTCC
GGGCCCAGGCCCTCCTCCAGCGACACCCAGGCCTCGATGTAGATGCTGGGCAGCCCCCACCACTGCACCGGGCCTGTGCCCGCCACGATGCCCC
TGCCCTGTCCCTGCTGCTTCGGCTCGGGGCTGACCCTGCCCACCAGGACCGCCATGGGGACACGGCACTGCATGCTGCTGCCCGCCAGGGCCCA
GATGGTGAGTCTGCTCAGTGGGGAACAAGGTCATAAGCAGCTGACCAGACCTGAAATGAAAGCCAACCAATAGTTGAGAAATAAGCTGGTTATT
TGGTCATCAGGACCTAGGGAAGGAGTTAACCAAGTTGGCATGTGGCTGTCATTTGTCCCTTTACATTACTGAGCTACCATTGTCTGAAGAACCC
AACATTCCCCAAAGATCAACTGGTCTTCAAATTTCACATCTGTTTAGATTAGTAGCTACTTTGTTTCTTGACAGATTGTTTGCTCGTAGCCAAA
AAGTAGCATAGAAGGTAGGCTCTGGAGTTAGATTGCCTGGATTCAAACCCCAGCTCCAAATCCCAGCTCCACACTTCATAGCTACGTATTCTTG
GACAGGTTACTTGAGGCTTAGTTTGCCCATGTGTAAAAATTAAAATAATAACAACCTTTGCTATGTGCCAGACATTTCTTATAAAGTAACACAT
TTAATCCTCACAACAATCTTAGGAGGTGAGTACTGATATTATCCCCCATTTCCCAGCTGAGGAAACAGGGCATAGAGAAGTCATTTGCCAGAGT
TACAGTTATTCACTGGTAGAGCAGAGATTATAACCCAGATGGACTAGATAGAGTGTCCATGCTTTTAACAGCTACATTGTCCTGTGTTATACAT
TATAGCATTGTACATTGATTGTGCCCATGTTCAGAGTACCCATGTTGTGCCATATATGTTTTGAGAATCAACTGACATAGTACATAATTAGAGT
ACCTGGCACACACGATAAGCACTTGGTATATGCTGGCGATTGTTGTTCCTGTTTCTCTGTTTTTTGTTTTTGTTTTTGTTTTTTATGAAGTTTC
ACTCTTCTTGCCCAGGCTGGAATGCAATGGTGCGATCCTGGCTCACTGCAACCTCTACCTCCCAGGTTCAAGTGATTCTCATGCGTCAGCCTCC
CAAGTAGCTAGGATTACAGGCGCATGCCACCACGCCCAGCTAATTTTTATATTTTTAGAAGAGATGGGTTTTCGCCATGTTGGACAAGCTGATC
TCGAATGCCTGACCTCAGGTGATCCACCAACCTCAGCCTCTCAAAGTGCTGGGATTACAGGTGTGAGCCACCACACCTGGCCTTGTTCCTGTTT
TTGTTATCAACAGGTCCATACTCCCTTAACCACAATTCTAAACTCAAAAACACTCTGAGAACCAACATTTTTCATCAGGCTGCCACCAAAATTC
ATTTGGTGACAGAAACCTAATCTGAACTAAAGTAAGACTATTATTTATTTTCATCCTACTGATGTCAATATTCATACATTTCCCTGCAGAAACA
CTCATGTGTTTGGTTCTTGGGCTGCCTAGGCCCTCCTGGGCTACCTAATATAGAGTGAGTGTACTTTTAGGTCAGCCCTATCAAGTCCCAAAAA
CATTTGAATTCTGCAAAACCTTTGGCACTGAAGGATTCAAATGGGGAACCTGGTGATATTATAATAGTGGTGGAGGCCAGGTGCGGTGGGTCAT
GCCTGTAATCCCAGCACTTTGGGAGGCCAAGGCAGTCAGATCACGAGGTCAGGAGTTCGAGACCAGCCTGACCAACATAGTGAAACCCCCATCT
GTACTAAAAATACAAAAATTAGCCAGGCATGGTGGCACACACCTGTAGTCTCAGCTACTTGGGAGGCTGAGGCAGGAGAATCACTTGAACCCGG
AAGACAGAGGTTGTGGTGAGCCGAGATTGCACTACTGCATTCCAACCTGGGCAACACAGCAAGACTCCGTCTCAAAAAAAAAAAAAAGAGTGGT
GGAAGCAGCTCTTTATAGGTAGAGCCCTGCTTACTAGAATAAAAGCTGAAACCTTCTTTCCCCATCTAGAGATTTCCTTCTGGAGTAAGAACAT
TACAGGAAAACCTCTAGATCCAGATGAACAACCCTAACATCCCCCAGCTCAAGTATAGACAGAAGGCCCCTCCCCCAAAACTCCCCCAAATGGT
CAAAAAACCCCCTATTTAAAAATTTCCTTTAACGTACCTGAGATAGGCTAGCATATTCAGATTTGTTTCTTGTTGTTTTTACTTAAAACAGAGT
AGGTTTACTGAGTGCAGGCATCTAACTTGACAGCTCATATTGTAAGAGGCAGGACCCTGGAAGGCAAAAGAGCAGATTACCCCGAAGCAGACCT
GCATCCAGACCCCAGCTCTGCCATCAACAGGGACATGCAGCTTACCTCTGTGAGCCCAATTTGCCTCGCAAAAATGGGAGTTTTGTTTTTTGTT
TTGTTTTGTTTTTTTGAGATGGAGTTTCCCTGTTGTTGCCCAGGCTAGAGTGCAATGGCGCGATTTCAGCCCACCTCAACCTCTGCCTCCTGGG
TTCAAGAGATTCTCCTGCCTCAGCCTCCCAAGTAGCTGGGATTACAGGCATGCACCATCACGCCCGGCTAATTTTGTATTTTTGGTAGAGACGG
TTTCTCCGTGTTGGTCAGGCTGGTCTCAAACTCCCGACCTCAGGTGACCTGCCAGCCTAGCCTCCCAAAGTGCTGGGATTACAGGCGTGAGCCA
CCGCGCTCAGCCAAAATGCCCCTGATAGTGTGGTAGGGATTTCCTTTATTGTTTGTTTGCTTGTTTGTTTTGAGACAGGGTCTCATTCTGTCTC
CCAGCCTGGAGTGCAGTGGTGCAATCATGGCTCACTGCAGCCTCTACCTCGTGGGCTCAAGCAGTCCTCCCACCTCAGCCTCCCTAGTAGCTGG
GACTACAAGCACACACCACCATGCCCAGCTAATTGTTTGTATTTTTGGTAGAGACTGTTTTGCTATGTTATCCAGGCTGTTCTGCATCTCCTGA
GTTCAAACAGTCTGCCCACCTCGGCTTCCCAAAGTGCCGGGACTAGAGGCGTGAGCCACCACACCCAACTCCATTGTATTGAATTTTAAGAAGC
TGGTGAGACTGATATTATCCCATTTACAGATGAGGAAAGCAGGGCCCAAAAGGTTCGGGAACTTGTCTGAAATCTCACAGCTCTCAGGTCATTG
TCTTCCAAAGGGGGACCCAAGCTCAGTGCCTTCACTCCCAGACCCTGGTGTCCTCTCTGGCCTTATTTACTCCTGGTCCTCTGCCAGCCCTGCC
ACCAGATGGCCTTCTAACTCCTTGGTTGAAAGGCCCATCTCATTCAGCTTCCAGCTTCCTTTTTCTTTTCCTTTTGAGACGGAGTCTTGCTTTG
TCGCCCAGGCTGGAGTGCAGTGGCATGATCTCGGCTCACTATAACTTCTGCTTCCTGGGTTCAAGCGATTCTCCTGCTTCAGCCTCCCAAGTAG
CTGAGATTACAGGCACACACCACCATGCCCAGCTAATTTTTTTATTTTTATTTATTAATTTTTAAATTTTTATTTGTTTATTTATTTTTGAGAC
GGAGTCTCCCTCTGTTCCCCAGGCTGGAGTGCAGTGGCAGTATCTTGACTCACTGCAACCTCCGCCTCCTGGGTTCAAGTGATTCTCCTTCCTC
AGCCTCCTGAGTAGCCGGGACTACAGGAGCCTGCCACCATGCCCGACTAACTTTTGTATTTTTAATAGAGATGGGGTTTCACCATGTTGGCCAG
ACTGCTCTCGAACTCCTGACCTTAGATGATCCACCTGCCTCGGCCTCCCAAAGTGCTGGGATTACAGGCATGAGCCACCATGCCCGACCTAATT
TTTGTGTTTTTAGTAGAGATGGGGTTTCAACATGTTGGCCAGGCTGGTCTCAAACTCCTGACCTCAAGTGATCCACCCACCTCAGCCTCCCAAA
ATGTTGGGATTATAGGCATGAGCCACCGTGCCCATCCCACAGAATGTCTTTTGGTTTTGTTTTTGTTTTCTGTTTTGTTTTGTTTTGTTTGAAA
AGGAGTCTCATTCTGTCGCCCAGGCTGGAGTGCAGTGGCACAATCTCGGCTCACTGCAACCTCCACCTCCCAGGTTCAAGAGATTCTCCTGCCT
CAGCCTCCCAAGTAGCTGGGACTATAGGCGTAGGGACTGTAGGCGTATGCCACCACGCCTGGCTAATTTTTTGTATTTTTAGTAGACACGGGGT
TTCACCATGTTAGCCAGGATGGTCTCGATCTCTTGACCTTGTGATCTGCTCACCTCAGCCTCCCAAAGTGTTGGGATTACAGGCGTGAGCCACA
GCGCCTGGCCAAAATGTTTTTATGTTTATTTTTCTTAGTATGAAACTCCAGCGTATTAAAGAGCATTGAGAACGGTTGATCTGGTAAATCGCTA
TAAAGGCGGCATTTCTTTTTTTTTTTTTTTTTTTTTTTTTTGGCGAAGTGGGGGATGGAGTCTCATTCTGTCGCCCAAGCTGGAGTGCAGTAGT
GTGATCTCGGCTCACTGCAAGCTCCGCTTCCCAGGTTCAAGCCATTCTCCTGCCTCAGCCTCCCAAGTAGCTGGGATTACAGGCGCCCGCCACC
ACGCCCAGCTAATTTTTTGTATTTTTAGTAGAGACAGGGTTTCACTGTGTTGGCCAGGCTGGTCTCGAACTCCTGACCTCATGATCCGCCCGCC
TTGGCCTCCCAAAATGCTGGGATTAGAGGCGTGAGCCACCGCGCCAGGCCTAAAGGGGGCATTTCTAATACTGGAGAAAGGTGAACTTTTTTTT
TTTTTTTTTCCGAGACAGAGTCTCGCTGTGTCACCCAGGCTGGAGTGCAATGGCGCAATCTCAGCTTGCTACAACCTCCGCCTCCCGGGTTCAA
GCAATTCTCCTGCCTCAGCCTCCTGAGTAGCTGGGCACCTGCCATCATGCCCAGCTAATTTTTGTATTTTTGTAGAGATGGGGGTTTCACCGTG
TTGGCCAGGCTGGTCTTAAACTCCTGTCCTGACCTGAGGTGATCCACCCACCTCAGCTTCCCAAAGTGCTGGGATTACAGGCATGAGCCACTGT
GCCTGACCAGGTGAACTATTTTATAAATAATATTGGAACATTTGGCTCATCTAGGGAAAAACAAGAGTCCCACCTCACACCTAATCAAAAAGTA
AATTCCAGCAGATTAAATACCTGAATATGACAGGAAGGTACACACCAAATTCATGGGACAGATGGCCTGGGGAGGAATGAAACTTGGAAGGCGG
TATCACGGTAAACTACCTTTATCTGTGATGATTTTATTTCCTTAAAATAAATTATACTACAAACATGACAGTACATTAACAAACCCTGTGGTAG
GAATGGGGTTGTGTATTGTATTATACTTTGTATTTTTGAGAGTTTTTTAATTTCTTTTTTGTTGTTGTTGAGACAGAGTCTCACTCTGTCACCC
AGGCTGGAGTCCAGTCGCGCAATCTTGGCTCACTGCAACCTCTGCCTCCCGGGTTCAAGCAGTTCTCTGCCTCAGCCTCCCAAGTAGCTGGGAT
TACAGGCATCCGCCACCACGTCAGGCTAATTTTTGTATTTTTAGTGGAGACGGGGTTTCACCATCTTGGCCAGACTGGTCTTGAACTCCTGACC
TCATGATCCACCCACCTTGGGCTCCCAGAGTGCTGGGATTACAGGCATGAGCCACCGCGCCTGGCCGAGTTTTTTAATTTCTAAAAATAAAAAT
GAGTATACATCTAAAGTAGTAGAAGAAAATGCAGAGAATGTTTTATAATCTTAAAATAGAGCCTTCCTAAGAGTGAAGTGAAATAAAAAGTCAG
AAAATAATATATTGATATAGATTTAACTACATGAAAATTTTAGGCCGGGTGCGGTAGCTCACACCTGTAATCCCACACTTTGGGATGCCAAGGT
AGGCAGATCACTTGAGCCAGGAGTTCAAGACCAGCCTGAACAACACAGTGAGACCTGGTCTGTACAAAAAATACAAAATTAGCCAGGCGTGGTG
GTACGTGGCTGTCGTCCTGTAGTCCCAGTTACTCAGGAGGCTGAGGTGGGAGGATCGCTTGAGCCCAGGTGGGGCAGAGTTTGCAGTGAGCAAG
ATCATGCCACCGCACTGCAGCCTGGGCAACAGGGTGAGACCTTGTCTCAAAAAGAAAAAAAAAGCTTTTTTAAAGATAGATAGAAGAAAATGTT
TGCAATATGTATAACACATACAGTATATAGAACCCTCATATCAATATATATAATTTCCAAAGAAAAAGTGGATAAAGAATATGAGCAATTCATT
CACAAAAAATACAAATAGCCAAAAGACATGAAAAAGAAAAAAACATTGTTAATAAAATAATTTTTTAATCTATCCAACTGGCAAAATTAAAAGG
GTTGATGATATTCAATTTTGGTAAAGGAAGACATAGGCACACTTGTATATTGGCACAAACTTATTGAGAGCAGTTTGACCAAATTGCGCATGTC
CTTTGACTCAGAAATTCCACTTATGAAAATCTACCCCCACAGAAAGACTGTAAGATACTCATGAGGCTGAACATTTTTTGAAACAAAGTCTATC
GATATTGGGGTAAGGAGATTTGTTTTGTACACACAGTGGAACACTAATTATAAAGTCGTCAAAAAGTATGAGGTAGACTGTATATATTCCTGTG
GAAAAATATAAATGAGATGTTAAATGAAAGAAGCAAGTTGCCAAGCAATATTTGTAGTATGGTTACATATCTCCAAATAAATGTGTCATATGTA
TGCTTTTAGGTACACCAAGGTGGGACTGGAAGGGATCACAGTACACTGTTAATAGTTATCAAATTTGTTCGTCCTTCTTTAAAAAGAAAATTTC
AACTTAATTATATCCCTTTCACATTAAAATGTCAAATAAAATTGGGGGAAAAGCCACCTACAGCCCCACCACCCATAGGCTAGAATTTTTACAT
ATTTTTTGCCAGTCTATTTTTTTTCTTTTTTTTTTTTTTTTTTTGAGATGGGAGTCTCCCTCTGTTGCCCAGGCTGGAGTGCACTGGCATGATC
TCAGCTCACTGCAACCTCTGCCTTCCAGGTTCAAGTGATTCTCCTGACTCAGCCTCCCAAGTAGCTGGGATTACAGGCACATGCTACCACGCCT
GGCTAATTTTTGTATTTTTAGTAGAGACAGGGTTTCACCATGTTGGCCAGGCTGGTCTCGAACTCCTGACCTCAGGTGATCCACCCGCCTCAGC
CTCCCAAAGTCCTGGGATTACAGGCCATGACCCACCACGCCCGGCCTCTTTTTTTCCAACCATAGGATTTGGTCTCTTGTTTTAGACAGTTATT
ATCTGATCCTCTCTCTCTCTCTCTTTTTTTTTTTTTTTTTTTTTTTTGAGATAGAGTCTCACCCTGTCACCCAGCCTGGAATGCAGAGGCGCGA
TCTCGGCTCACTGCAACCTCCGCCTCCCAGGTTCAAGCAGTTCTCTGCCTCAACCTCCCAAGTAGCTGGGATTACAGGCGTCAGCCACCACGCC
TGGCTAATTTTATATATAATATAAAATATAATTATATATATATTTTGTTTGTTTGTTTTAGTAAAGACGGGTTTTCACCATCTTGGCCAGGCTG
GTATTGAACTCCTGACCTTGTGATCCATTGTCCCCCCCCCCAGCCTCCCAAAGTGCTGGGATTACAGGCGTGAGCCACCGTGCCTGGCCACATC
TGATCCTCTCTACAGTTTTGTAGCCAGCTTTTTTCAAACACATGCCTCAGTTGTAGTGGCTGTTTAATATTATGTTTTTATAGTTGGAGGCCCT
ACTCCTTAAAACCTATGAATGTAAACCTCCCATGCAAGCCTGAGCACTCACCATGCTCACCACCTGAGCTCAGGTGGGGAACAAGCGAATGAGA
GACAGGACCAGGTACTTTCTGGGTGGGACAAGTTGAGAGGGTCTGTGACAGGTCACAGCAGCACTAGGGAGAAGTGCCCCCCCCACCAACCCTG
ATGTGATTTGGGTAGGGATGGTGGGCCTTCGCCAGCCACACCTGGGCCATTCTGTCTTCTTGCCTTCCTGGCCTTGCCTTCCCCTATTCCAGCT
TTCTGCCAGGTAAACAGTACTTTCCAGCACTACCAAATAAAGATTTAAGGACTGCTAGCCCATTTCCTCTTACCCCGGGGAAAGAAAGTAGGTC
CACAGGAAGGAAGGCTGCCTCCCTCCCCTTCTTCTATCCCCCAAGTGAAAGAGGTGGTTGGTGGCCACAGCAGGTGGGCCTGGCCAGGATGCCT
GGGTTGGCAGTGAAGGAAGTAGCATGGCACTCAGCTAACCTTGGGCCAGATGCAGCAAGGTTGGGACTGAAGAAAAGGGGAGTTCAGGAACGTC
GGTTCCTCTCCTGTTTTCTCTCAGCCCATGGGTGAGACCCTCTGTCACACTCCACTCCCTTTCCCCTGCCCTAGGTCAGCAGTCATTTGGAAAG
AGCTTGCTCTGCCGCCGGCCATAGTTGTTGCCGGTTCACCTCCCCACCCCTCTCCCATCATCCCCTGGTAAGGCTGGCTGAGAGAAATTCCCCT
GAAAACATTTATTTTACTCAGTTTATTGATAAGTGATAATAAAAGATAAGTATTACATATTTGTGTATTTATTAATGGCCCAGAGTAGAGCATT
CAGATATTTTCCCAGTCTGATATATGGTTTCAGGTGAGAAAATAAGGGATTTATATAGTGACAATATTTTTAAGTGAAAAGTGGAATACATAGT
CAAAGAATTTGGGCACTGCCTTGGTGGCTGGAGGCAGGTCAGAAAATGTCAGTTGGCATGGTAGAACTTCTAGGATGCTGAAATAGTTTGTGGA
GACACAAGTAAGAATTTTTTTTTTTTTTTTTTGAGACGGAGTCTTGCTCTGTCACCTAGGCTGGAGTGCAGTGCCACGATCTCCGCTTACTACA
AGCTCCACCTCCCGGGTTCACATCATTCTCCTCCCTCAGCCTGCCGAGTAGCTGGGACTGCAGGCGCCCGCCACCAGGCCTGGCTAATTTTTTG
TATTTTTTAGTAGAGATGGGATTTCACCATGTTAGCCAGGATGGTCTTGATCTCCTGACCTCGTGATCCGCCCGCCTCGGACTCCCAAAGTGCT
GGGATTACAGGCGTGAGCCACCGCGCCTGGCCGAGAATATTTTAAACTACCACACTTACCATGCATGTGGTAAACTATCAGTCTGTATTTATCA
GTGATGGTTATTTCCTAATACCCAGGAGGCATCCATGTGAGCCACCCTTCCATTGCTTTAAGACCAAGGGAGCGAGTGACCAGCAGGATTCAAG
ATGGCAGCTCTGCCGAGGAGTGGGAGTCCCAGCTAACTTCTGCTCCCTGCTCTCCCACCAACAGCCTACACCGATTTCTTCCTCCCGCTGCTAA
GCCGCTGTCCCTCCGCCATGGGAATAAAGAATAAGGATGGGGAGACCCCTGGCCAAATTTTGGGCTGGGGACCCCCCTGGGATTCTGCTGAAGA
GGAGGAAGAAGATGATGCCTCCAAGGAGCGGGAATGGAGACAGAAGCTCCAGGGTGAGCTGGAGGACGAGTGGCAGGAAGTCATGGGGAGGTTT
GAAGGTGAGAAGTCCACTGCTATCCACAGCTGCCCTTCCCCACTGGCTGCTTTCCATCTGCATGAATGCGTCACACTAGGCTCCTCTGCCCCCT
CCTCTGTGCTTCCCTGCTTCTTGGGGCCCATCACCTTCTCACAGCCTCTCTCCAACTACCCCCATCCCACCCTCCCAAACAGGTGATGCCTCCC
ATGAAACCCAGGAACCTGAGTCCTTCTCAGCCTGGTCAGATCGCCTGGCCCGGGAACATGCCCAGAAGTGCCAGCAGCAGCAGCGAGAAGCAGA
GGGATCCTGTCGACCCCCACGTGCTGAGGGCTCCAGCCAGAGCTGGCGACAGCAGGAGGAGGAGCAGCGGCTCTTCAGGGAGCGAGCCCGGGCC
AAGGAGGAAGAGCTGCGTGAGAGCCGAGCCAGGAGGGCGCAGGAGGCTCTAGGGGACCGAGAACCCAAGCCAACCAGGGCCGGGCCCAGGGAAG
AGCACCCCAGAGGAGCGGGGAGGGGCAGCCTCTGGCGATTTGGTGATGTGCCCTGGCCCTGCCCTGGGGGAGGGGACCCAGAGGCCATGGCTGC
AGCCCTGGTGGCCAGGGGCCCCCCTTTGGAGGAACAGGGGGCTCTGAGGAGGTACTTGAGGGTCCAGCAGGTCCGCTGGCACCCTGACCGCTTC
CTGCAGCGATTCCGAAGCCAGATTGAGACCTGGGAGCTGGGCCGTGTGATGGGAGCAGTGACAGCCCTTTCTCAGGCCCTGAATCGCCATGCAG
AGGCCCTCAAGTGACCCTAGGGAAGAAGCAAGAAACTTCGGGGCTGCAGCCTCAGGATGAGGCAGAAGGAAGGGTAAGGGAAAGGATGGGGACC
ACAAGGAAGAGCCAGGTGCTGCTCAGCAGAGGATATGGGTGGGAGCGAAAGTTGTAACAAGTGGGGGTGGGGGGTGCGGGCCGCCACCACTGCT
CCTTGACTCTGCCGTTTCCTAATAAGACCTGGTTCCACATCTCACTCCCAGTGTCTCCTCTGTCTTTTTCCATTGCTGTGGTTTTCATCACCCA
TGACATCTCCTTTCCCGCCCCGCCTGCTGAAACCCACAGCTCCCACACACCTGCAACACACACGCACACGCTAACACGGGCTCTGAGCTGGAGG
CAAGAAGCCTCTGCATGCCCCCTCAGTTCAGCCCTAAGAAGGCCCAGTTTGCCATCCAGTCTCACTCCACTCCCTACACTGGGGTCTTGTCCAC
CCTGCAATCTGTGGCTGGAGAAATAGATGCGAACAGAGGCAAAAAGGGGAACAAAACCAGTTTCCCTCCCCTCCCTGGCTCCCCAAGCTGAACC
ACATCCTCCTCCCCACTTAACACCCCCTTCCCCCAACACAGGGCTTTCCCTTTGCTGAGTCACTGAATGAGCGAGTTGGGGGTAGCCGGCGCTG
GGGGGCCATGAGGAGGCTGGGGGAGGATGGGGAATACAAGCAGAATGGCTGGAGGAAGAGCCCTGTGGGGGAGTGGAATTTCAGTTGCTAAAAT
TAGGAGCAGGGGAAGGAGGTGGAAAGAGCAAAATTATGTAACATGGGTTGTCTGTTCTTGGGCAACTGGAGCTCCACACCCAAAGCCAGCCAGG
CTGCTGGCTCCATCCATCTCTGCCCTCTAGCTTGTCAGTTGTATCTCTCTTCCTCCAGGGCCCCAATCCTCATCTCCGCCATTCAGCTGCTGCC
CCATCCTAAACCTGAGTTCATCTCTGGGCAGCCCAGGCATGGCCTTCCCTATAAACATTTCCTTTTCCAAGAACCAGTAGTTGAAGTCCTGAGA
GGTGGAGGGAGAGTCTGGGATTCCCACGGAGGAGAGAGGGGGGCTCCCTGGAAACTAAGATAGGTAGACCCCACTACCATCGCCCAGGACACAA
CTGGGAACTTGGCAAAAAGAAAGGACAGGGCTGCAAGGAGAGTACAGACATGTGCTGGTGAGTGCACTGTCTGCATAGTTACACCAGAGCATCT
TATCAATCAGAAACTTATCTTTCAGGTTTTGAGCCCAGTTCTCTACAGGAGAATCCCAGGAGTGGAAGTGGAAGGCAGTAGAAGACAGGGAGGG
CACGCCTCTGGGAACACGGGAACATGGGTGGGCATGAGATCCTTGAATAAGACAGCCTGAAGTTCGGAAGAGACCAAGGCCTCTGAAGGACCAG
GCAGATGTTCAGGGTGCAGGAGGGGGAAGGGCTGGTGAGAAAGATCCTGTGAGAGGAAGCTGCTGTGATTCAGAGAAGAGACTTCAAGCTGTGT
GTGACCCTGGCGTCCCGTTCCTCTCACAGGCTGGAGCTTTTCGGAAGTGGCATGCAAAGAGTCCAGGTTTGGCCTTGGGGGGAGTTGGGGTTAG
GATCCCTAAGCTGGAGGTTGAGAAGTAAATTACAGAAAACTCTGGTGACCAAATTTGCTCCTCCACCCAGGAGATTTCTCACTGGTTTTTAAGC
ACATCATTTCCCCTTCTGCAAGAGTTACATAAAACCAAAGCAAAATAAGCCCTGAAACCTGGGCCCACCGGACCACAGTCTTTTCAACGTCCCT
TCCTGGTGTCTGGCCCCCAGCCCTGGTGGGGGTTCCCCTGAGATAAGGGCTGTTCACTTTCTCTGACCACATGGTTTCCGCTTCTGTGTCTCTT
GTTTCCTAGGCTGATAAAAATACTGAGCCCTAGAGGCCCTGGCTTCCTCTGACCCCTTGGGGCAGGCAGCAGGCATCCTGTCCAGCATGGTGGG
GGCAGGGACAGGGGCCAGGGATTCCCAAGGGGTGACTCAGTGCCTGCCATGAAACAGTGGGTAGGTGGAAGTGTATCTCTGCTCTCTAGAGCTG
GCACCAGGAGTTGAGTCTCAGTGGAGGATGCATTGGGATTCAATTGGAGGAACAGGCCTGGAAAAGAATAATGAGATTGAAGAGGGTCAGTTTG
AGGACTCAGGTTGGGGCAGGTTTGGATTAAGTTAGGAAAAGGATCTGGGAGGGACTCTGTTCAGTGTAGGTCAACTGAGCATTATGTAGCCCAA
AGATAAATTTAAACCCTGCTTCAAGCTTACAATCTAGTGGGGAGGCAGACCCATACCTAATTAACTGATTCAAGGCATGATGAGTAAACTTTAA
GATGATTACAGAAAGAGGGGAGATTAAGTCCATTTGAAAGCATCCAGGGAGCCTCTGAGGAGACAGCATTTGAACTTGCTCTAATGAATGGGTT
CACTAGGTGGAGGTGGATGGAAAGGTTCCATAGGCGAATAACACGTCTTGAGCGAGCCTGACAAGTCAGAAAATGCAGTATGTTCTGGGAAAGG
AGCGTCCTGAGGGAGAAGAAGCACAGGTGTGAGGGAACATGTGATGAAGAAAGGACCACAGAAAGTCAAGGTCAGAGATTGGACTGCATCCTGT
GGGCAGTGGTCCAGGTGACGATGAAAAAGAGGGAGAACAGGTGAGTGCTGCAGTACAGACAAGGAGTAAGAAAACGGCCATCATCTTGTGAATT
AATACCTACTGTGTGTTAACCAGCCCTTTTCCTAACACCACAAATCCTCTCAACGTCTGTCCAAAAGGTGGGTGGTGGTGGCCAGGCACCTCAC
TCCTGTAATCACAGCACTTTGGGAGGCCAAGGTGGGAGCACTTTGGGAGGATCACTTGAGGCCAGGAGTTCGAGACCAGCCTGGCCAACATGGT
GAAACCCCGTCTCTACTAAAAATAAAAATACTAGCTGGGTGTGGTGTTGGGCACCTGTAATCCCAGCTACTCAGGTGTCTGAGGCACAAGAATC
ACTTGAACCCGGGAGGCAGAGGTTGCAGTGAGCTGAGATCATCCCTCTGCTCTCCAGTCTGGGTGACAGAGCAAGACTCTGTATCCAAAAAAAA
AAAAAATTATTAAGCACCTATTAGGAGCAGGGCACTGCTTGACAATAGGTATAAATAATAAAGTCACTGCCTTCATAGAACTTGCAGTCTAATG
AGACAGTATACAAATAATAACTATACATTATAGTTATAATGTATAGGTATGCCTGCTTAGGGTAATAAAGTGCTCAATGAAGGTTTGAGGTACC
AGCATGACTCTCAGGTGGAGATTTCCAGAAAGCAGGTCTGGAGCTCAAGAGAAGTTGGGTCTGGAGGAACAGATTTGGGCATCATTCCCTTCCC
AGTAGAGGTTGAGCCTTTGAGTGGACAGGATCTTCAAGGGAGGGGGCGGAGTGACCAGAAGAGCCCTGAGCATGATCAAAGGAAGAGAACCAAT
AAAGGAGAGGTTGGGGAGCAGTCAGAGAAGCAGGGCACGGGGGGCGGGGGATGCCAAGCAGAGACAGGGCAGCCATGTTTGAGGCTTCAAAGAG
GTCTAGTAAATGAGGGTTGAAAAGATTGTTGGGTTCAGGAACTAGACGATTACAAATTTTGAGAAAACCGTTTCCATTTAATAGAGGGGCAGAA
ATCACTTTACATAGGTTGAGGAATGAGTGGGAGGTGAGGAAAAGGAGGTGGTGGGCATGAGGTCAGAATGGTGAACACAGAATAACTGAGAATC
ATCTCTTAGTTCTACCCACAGATTTTACAGTTGAGGGAAATTTTACCAGTTCCTGAAAAAGTGGTTCGTTAAGGGGGCTAGTCTTTTGAGACAT
CACACGAAAACGGAAGGTGAGATAGACTGGACATTTGAGGGAGAAATATTACCAAGGAAGGATCACTACTTC
HUMAN SEQUENCE—mRNA
CCGAGCTTCTTAAACACAGGCCTTGGGCTACGGCTCTGGGGGTACTTGGGGGGGCGGGGGCAGGTCTGATGAGTAACCCCTCCCCCCAGGTTCC
AGAGGAAGAAGCCTCCACATCTGTCTGCCGGCCCAAGAGTTCCATGGCCTCCACTTCCCGCCGCCAACGCCGAGAACGTCGCTTTCGTCGTTAC
TTGTCTGCAGGACGGCTGGTCCGGGCCCAGGCCCTCCTCCAGCGACACCCAGGCCTCGATGTAGATGCTGGGCAGCCCCCACCACTGCACCGGG
CCTGTGCCCGCCACGATGCCCCTCCCCTGTGCCTGCTGCTTCGGCTCGGGGCTGACCCTGCCCACCAGGACCGCCATGGGGACACGGCACTGCA
TGCTGCTGCCCGCCAGGGCCCAGATGCCTACACCGATTTCTTCCTCCCGCTGCTAAGCCGCTGTCCCTCTGCCATGGGAATAAAGAATAAGGAT
GGGGAGACCCCTGGCCAAATTTTGGGCTGGGGACCCCCCTGGGATTCTGCTGAAGAGGAGGAAGAAGATGATGCCTCCAAGGAGCGGGAATGGA
GACAGAAGCTCCAGGGTGAGCTGGAGGACGAGTGGCAGGAAGTCATGGGGAGGTTTGAAGGTGATGCCTCCCATGAAACCCAGGAACCTGAGTC
CTTCTCAGCCTGGTCAGATCGCCTGGCCCGGGAACATGCCCAGAAGTGCCAGCAGCAGCAGCGAGAAGCAGAGGGATCCTGTCGACCCCCACGT
GCTGAGGGCTCCAGCCAGAGCTGGCGACACGAGGAGGAGGAGCAGCGGCTCTTCAGGGAGCGAGCCCGGGCCAAGGAGGAAGAGCTGCGTGAGA
GCCGAGCCAGGAGGGCGCAGGAGGCTCTAGGGGACCGAGAACCCAAGCCAACCAGGGCCGGGCCCAGGGAAGAGCACCCCAGAGGAGCGGGGAG
GGGCAGCCTCTGGCGATTTGGTGATGTGCCCTGGCCCTGCCCTGGGGGAGGGGACCCAGAGGCCATGGCTGCAGCCCTGGTGGCCAGGGGCCCC
CCTTTGGAGGAACAGGGGGCTCTGAGGAGGTACTTGAGGGTCCAGCAGGTCCGCTGGCACCCTGACCGCTTCCTGCAGCGATTCCGAAGCCAGA
TTGAGACCTGGGAGCTGGGCCGTGTGATGGGAGCAGTGACAGCCCTTTCTCAGGCCCTGAATCGCCATGCAGAGGCCCTCAAGTGACCCTAGGG
AAGAAGCAAGAAACTTCGGGGCTGCAGCCTCAGGATGAGGCAGAAGGAAGGGTAAGGGAAAGGATGGGGACCACAAGGAAGAGCCAGGTGCTGC
TCAGCAGAGGATATGGGTGGGAGCGAAAGTTGTAACAAGTGGGGGTGGGGGGTGCGGGCCGCCACCACTGCTCCTTGACTCTGCCGTTTCCTAA
TAAGACCTGGTTCCACATCTCAAAAAAAAAAAAAAAAAAAAAAAAAAAA
HUMAN SEQUENCE—CODING
ATGAGTAACCCCTCCCCCCAGGTTCCAGAGGAAGAAGCCTCCACATCTGTCTGCCGGCCCAAGAGTTCCATGGCCTCCACTTCCCGCCGCCAAC
GCCGAGAACGTCGCTTTCGTCGTTACTTGTCTGCAGGACGGCTGGTCCGGGCCCAGGCCCTCCTCCAGCGACACCCAGGCCTCGATGTAGATGC
TGGGCAGCCCCCACCACTGCACCGGGCCTGTGCCCGCCACGATGCCCCTGCCCTGTGCCTGCTGCTTCGGCTCGGGGCTGACCCTGCCCACCAG
GACCGCCATGGGGACACGGCACTGCATGCTGCTGCCCGCCAGGGCCCAGATGCCTACACCGATTTCTTCCTCCCGCTGCTAAGCCGCTGTCCCT
CTGCCATGGGAATAAAGAATAAGGATGGGGAGACCCCTGGCCAAATTTTGGGCTGGGGACCCCCCTGGGATTCTGCTGAAGAGGAGGAAGAAGA
TGATGCCTCCAAGGAGCGGGAATGGAGACAGAAGCTCCAGGGTGAGCTGGAGGACGAGTGGCAGGAAGTCATGGGGAGGTTTGAAGGTGATGCC
TCCCATGAAACCCAGGAACCTGAGTCCTTCTCAGCCTGGTCAGATCGCCTGGCCCGGGAACATGCCCAGAAGTGCCAGCAGCAGCAGCGAGAAG
CAGAGGGATCCTGTCGACCCCCACGTGCTGAGGGCTCCAGCCAGAGCTGGCGACACGAGGAGGAGGAGCAGCGGCTCTTCAGGGAGCGAGCCCG
GGCCAAGGAGGAAGAGCTGCGTGAGAGCCGAGCCAGGAGGGCGCAGGAGGCTCTAGGGGACCGAGAACCCAAGCCAACCAGGGCCGGGCCCAGG
GAAGAGCACCCCAGAGGAGCGGGGAGGGGCAGCCTCTGGCGATTTGGTGATGTGCCCTGGCCCTGCCCTGGGGGAGGGGACCCAGAGGCCATGG
CTGCAGCCCTGGTGGCCAGGGGCCCCCCTTTGGAGGAACAGGGGGCTCTGAGGAGGTACTTGAGGGTCCAGCAGGTCCGCTGGCACCCTGACCG
CTTCCTGCAGCGATTCCGAAGCCAGATTGAGACCTGGGAGCTGGGCCGTGTGATGGGAGCAGTGACAGCCCTTTCTCAGGCCCTGAATCGCCAT
GCAGAGGCCCTCAAGTGA

TABLE 23

MOUSE NOMENCLATURE
ICSGNM Flt3
Celera mCG12743
HUMAN NOMENCLATURE
HGNC FLT3
Celera hCG96642
MOUSE SEQUENCE - GENOMIC
GCTCTGTACAAGGTCATTTTCCAGACAAGGCCAGCCACCTGTGTTCAGGGACACTCTCCAGACAGAGACAGGGAAGCCCCACTAAGAAAGGGAG
ACCCCATTTTGCTCTATCTGGTCTTGGTGGACTGCTACCATCATAGCGAGGTTCTCCAACAGCTTTCCTGCTCAGTCACATATACTCAGATGTC
ACCGGCTTTTCTCAGACCACATTGCAAGGGAACCCATCTGTGTATCTGACTTCGTTCTTCATGGACTGTAGCTGCCTATGGATGGGGCAGTCCT
GACGTCTATCCTTGGAGTCCCAGAACCTGGTACTAGACACGTGCCTAATGAATCCTTGTTCTGGGAATGTCCTTAGTGAGTCATGACAGTGGGC
ACTGTACTCATGCTGGGTGCCTAGGGATTGGGATGCCCGTGAGGGCTTCAGCGTAACAGCCACACATAGGTGATGCCACATAGTGTGCAGGCTT
TCGTCACGTGAGCAAGCCGAGGAAGCAGAGCAGTATAGAGGCTCACGGGGAGGCCTTGCTCATGTATATCACTCACCACATGGCTCTTTTACTT
GTACCTGGAGGAGATTATCTTACAGAGAGCCTGTCCCTGGGTCCTCAGCAAGAGGAGTATGTCCTCTCAGTTTCTGAGTCATCTCTCTAGCTCT
AAGATCTCATCCCTAATGCAAATCCTGTCTACAAAAGATGATGGGCTCTGTCTGAGTTCTTCTTCTGTTTCTTTTTTGGACTAGCCCAAACCAC
AGCTCAACTTTGTCTATGCTTTCTTTCATTAAACTGACAAATTTTTAAAAAAATCATGTATGGTAGGTCTCTATGTTGATCAGCCTGGTCTACA
AAGGAGGTTCTAAGTTAGATGGGGCTAGAACCATATATATATGTATATATATGTGTGTGTGTGTGTGTGTGTGTGTGTATGTATATATGTATGT
ATCACATATATATCATAACATGATATATGTGATATATGTATATATGATATATGTGTGTATATATGATATATATAATCCTTATACAAATATCAAT
ATCTAATTTGTAAATTTCCTTTTTACAGAAAAAGGGTTTATAAACGCTACCAGCTCGCAAGAAGAGTATGAAATTGACCCGTACGAAAAGTTCT
GCTTCTCAGTCAGGTTTAAAGCCTACCCACGAATCCGATGCANNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNCAATTCCCCGGCCCCCATCCCCGGTATCGAGCACCTCTGTCCCCGA
GCTATAGACTTAGCCCCCTCAGTCTGTGAGTTGATTTATTGTTGAGTCAGAAAACTGAAGACTGAAAGCAAACCGCACCTGTGACCAGAGCCAC
TTTTCCTTCTCTTCCGGGACTAAAGCCTGCTGCATGAAAACAGAATCCTGGCAGTTTTCTGTCAAGAGACTGTGGTTTCCTGTCTAAGTGCTGC
GCTCTGACAGTACCGGGCGGCTAAGTGCGGTTGTGAGTGATGGGGGAGTCGGACCTCCTTGCCAACTGAAAACATCTGCCCTAGGTGTACTGAT
TTATGTTCTCAAACACAAATGCAGAAATAGCCCGAGCATAAGTCACAAAAGAGGATCTCCTTCTCCTGGGGGTTTGTTCAACTCTGGTCTAGAG
TGTTCTGGAGGCGAACGTCTCATTCCTTCTTTGAAAAGCAAAAGCCTGCTTTCCCCGGTTTCTTCTGTTAACTATGGTGCCTCTTCTGTCTTTC
TCTCCTTTACAGATTTTAGTTTTAGCCATGTGTATGTGGGTGACAGTCTGTGTGTGTGTGACTGCGGTGTCCCTGGAGGCCAGAGGCTTGGGAT
TCCCCTGGAGTTGGAGTCACAGTGGCTGTGAGCTGTCCCACGTGGGTACTGGGAACCCAGCTCAAGTCCTCTGCAACGGCACTGTGGGTTTGCT
CTTATTCGCTGCACCAAATCTGCAGCACAGCTCATGGACAAACACTGTCTCAACAACAACAACAACAGACAGACAAAAAACCAAACACACAAAA
CAGGAATGTAGAACATCTCATTAATAATTTTCACACTGAGGCTATGCTTCAATGATTGTTTGAGGTATGAGAGTTACAGAGATCAACGAATGCA
CTCAGCTTTCCCCTCTCTTTACTTCATATACATATCTGCAGGGGACTTAAAAGGAAAACGAGGCAGCTGTCTGTTGCTCTGGTCACTGTGACAA
GAGCTGACCTCTTTCTACCCTCTACGCTGCTCCTAGTGTTTCTTTCATGATAGTATGCTTTTTTTTAAAAAATTCCCATAAGTGATTTATAATT
GGTCTTTGAGAAAGTGTGTGTGTGTGTGTGTGTGCACATCTATCTGTATCTCTCTCTCTCTATGTCTGTGTTTATTCTCTCTCTCTCTCTCTCT
CTCTCTCTCTCTGTGTGTGTGTGTGTGTGTGTGTGTGTGTGTGTGTGTGTGTGTGTGTGTAGGATGGATTCCCTGGATGGAACTGGACTTATAG
GCAGTTGTGAACCACCTCATGTAGGTGCTAGGAATCGAACTCAGGTCTTCTGGAAGAGCAATGCTTGCTTTAAACCCCTGAGTTCTCTCTCTAC
CTCTAAATTTGCCTAGGGGGGTAGGGGTGTCTTGTGGGGTGTATATGTGTGTGTGTGTGTGTGTGTGTGTGTGTGTGTGTGTGTGTGCGCGCGC
GCGCGCACCCATGTGCAGAGGCCAGATCAACATCCTCTGTTGTGCTCCACTGTTTTTTAAAACAATACTTCATGTAATGCTTATGAGTGTTTTG
CCTGCATGTCTATCTATGCACGTGTGTCTGCTGGGGCATCAGGTCACTTGGAAATGGAGTTATGGGTGGTTGTAAGCCACCATGTGGATCCTAC
GAATCATGCTTTTAACCTCTGACTCATCTTTCTAACCCTCTCAACTATTTTTTTTTTTTTGAGACAGACTCTCTCATTGAACTTGGATCTTACC
ATTTTGGCTAGACTGGCTGACCTCCATACCCCAGCACTAAGGCTACAGAGCACGCACTAAGGCTACTGCACTCAACTTTTACATGGTGGGGACT
TGAATTCAGATTCTCCGGCTATACTACAAGCTTTATATACAGAGCCACCCTCTCTGCTTCCTATTTGTATTATTTATTTATTTATTTATTTATT
TATTTATTTATTTATTTATTTATTGGTGGGGAGAAGGGGACAGTTTGAGAAAGGGTTTCTTTCTCTCTGTATCCCTGGCTGTCCTGGAACTTAT
TATGTAGACCCGACTGGACCTTACACTCACTGAGATCTTCCTGCCTCTGCTTCCTGAGTGCATCAAGCTATGCACCACAACCACACCAGGCTCG
CATAAGCTCTTATCCAGAACTTGAATTCCAGAAGGAGAAGACAGGGAGGAACGGATGACCATGGAGCCTGTTTTCAGGATAGCTGGCTTGCTGG
CCCCTCTGTGGTCGTGTGAGAGCTAACACAGCCTTAGGGACTCCTGCTGCATTCCAGGATGGTTGTGAACCTGACTCAGGTGAAGGAGGGGATA
AACCTGGTTTAACCAATGGTAAGGTCGCGATGCAGCAACACATTTAATCCTAGCAGTCCAGGGGCAGAGGTGGCTCATCTTTGAGTTCTAGGCC
AGCCTAGGCTACACAGTGAGTTCTAGGCCAGCCTGGACTATACAGCAAAGGGCCATGTGAATGGGTAAGAAGCTGCCATTTTCCTTCTCTCCCA
GAGGATGCATATTGTCACTGTGTAACCTGGTGACACACAGCAGACCCTCTTGGGGTATCACGGAGCTGTGTCTGCCATGCATGCATCAGGAGCT
GGGCTAGGTAGCCGCATTTCATCCTGAGGCTCCAACCTACAAGCCTGAACTCCAGACACGTCCCATCCCAGCTAGAGCCGTCCTGTGTCCCTTT
CATTTCCAGTCACCTGAGAGCAGCTTCCCAGATGTGACATCTCAGCTCTGAGTTTAGAACCTCCATAAAGATGGCCAGGCTGTCATCTCTCTAG
CTCCGTCCCGTGCCTCATACAGGAAATGTGTGTGTGAACCAGGAAACGGACTCACTGCCACTCCTGGACATGACTTCTTGGAGAAAGCTGTGCC
TGTGTTGTCTTGCGTACAGCACACATGGCGCCCAGTGCTTGGCGTGAGATTCTCTGAAGGTTCCCAGAGAATAAAAAACATGGCAGCAAGGAAG
CCTGAGGTAAAGCAGTCAGCGCTGAACAATGTTTTTCCGTGTTCTATGATCCTTCTCTCTGCTGTGTAGGCTCTGGTTGAGGTGAGAGGTAACT
CAAGCTCTATTCAACTTTAATGGTGGAAAGTTTCAAATTTGCTAATACACCAAAGGAACTGGCATCCACTCACTCTCACTCCAGTCCAAAGATT
TACTTATTACTATATGTAAGTACACTATACCTGTCTTCAGACACACCAGAAGAGGGCATCAGATCTCATTAGGGATGGTTTTGAACCACCATGT
GGTTGCTGGGATTTGAACTCAGGACTTTCGGAAGAGCCATCAGTGCTCTTAACCGCGGAGCCACCTCTCCAGCCCAGGAGCTGGCATCCTTAGC
TCCCCACTAGATTCTGCAGTTCTGTTGCCCTGAGTTGGGATGGTAGAGGATCGAGATCATGTTTTGAGAGCTGACCTGAGTGGCGGGAGAGTTG
CTGGTTTGTTGGTTTTTTGTTTTTTTTTAATCTGTGTAGTAGCTGAACTACTGCAGAAGCCCCGATGCGGTCGTGGCACACGCCAGAAGTCTCT
CGGCTTCCCGACCATCAAAGCTTCATTTCTGCAATTTACTTCCTCAAATCACACCCTGAGGCTCCCTGGGGCTTGCCTTTCCGTGTTTGTGTCT
GGTTTCCAGCTGCCTGGAGCCCTTTTACTTCTGAAAAAGAGACTGAAGGAGATTCACCCCTTACTTTGGGGGCGAATTCTGTAGACTCACCGAT
AAATACTGAATCCGCATTAGTGTCGTGGTAGAAAAAGTATGAGCATGGCATAACTTTTCCCTGGAGCTATTTCATCCTGTCTTCCACGTATCTC
AGTCCATTCTGTCCCCTCTGAGCTCCCACTGGGTGTTTCCAAAGTGAAACAGGGGCTCTTTCCTGTGTTCGCAGTCTGGAGGACCTGCTAGGGG
CCTGCTCAGGACAAGAGTGGCCACATAGAAGCTCCTGAACAGGACCTGTGGCAGAGCTGACATTTTTGTTTTTAATGGAATGGGGCTTGAGGGT
AGGAGAGCTGTGAGCCTGAGTATATACAAAAAGGAAGGGGAAGATGTTTGTCTCTAGAGCAACACCAAGAACCCAACTAAATCTGTTTTCCCTT
TATTCATGCTGCCTGTAACCGTGCTGCCTGTAACCGTGCTGCCTGTAACCGTGCTGCCTGTAACGGTGGCCCCGGCCCTTCCTTTCTGGGGCCA
CACAGCGAGACCCTGTCTTAAACATATGTAGTAAAGTGTATTTACTGTATGGTTATATTGTTATATCTTAGTTACCATTTTTTAAAGATTTATG
TATTTTATTTATATGAGTACACTGTAGCTATCATCAGAGGGTTTCAGAGCCCACTGCAGATGGTTGTGAGCCACCATGGGGTTGCTGGGAATTG
AACTCAGGACCTCTGGAAGAGCAGTCCATGCTCTTTTTTTTTTTTTTTTTTTTTTAACATCTGCCACTCTTTTTTTTTTTTTTTTTTACATTTA
TTTATTTATTATATCTAAGTACACTGTAGCTGTCTTCAGACACTCCAGAAGAGGGCGTCAGATCTTGTTACAGATGGTTGTGAGCCACCATGTG
GTTGCTGGGATTTGAACTCTGGACCTTCCCAAGAGCAGTCGGGTGCTCTTACCCACTCAGCCATCTCACCAGCCCCCAGTCCATGCTCTTAACC
ACTGAGCCATCTCTCCAGCCCCTTAGTTATAATTTTCGTAGCTATATAGTGTTAAAGGTTTTAAAAGTTGAAACAGGGTCTCACTGTACAAGCA
CTGGGTAGCCTGGAACTGGAGACATAGATCAGACTGGCCTCAAACTCACAGAAATGACCACCTACCTGGCCCTCCATCTGACACTCTTTATGAA
TCATAAGCTGATTCCAGAGCCCTTAGCCAAGGTTGGCCTTTTTTAACTCCTCCCCCTGGGAGCTAAATACAAGCAAGCAGAAAGTGACCTGATG
GTAGAGAGGCCCTCTTCCTAATTCTAATGACACTGCATTGTCTCGGTCTCCAAAGATATGTCACTGGCAACTCAAATTTCCTCATGCCAAGTGG
GAAAGGGGAGGTGGCGGAACCTGAAAGAAAGGACGCTCTGAGCTGCCTGGTACCTGACGGTCCCTCTCCTTGCTGCTGGGGTCATATTCATGGC
AGAAAGCGAGGTGTGGTGGCACAGACCTGTCACTCTAGAACACTCGAGGCTGAAGGCAGGCCAAATGCAGGTCTGAGGCCTCCCAAGGTCAGAC
AGACAAAAGAAAGTGGTAAATCTGTCTGGACATTTGGTTCTGTGTCCCGCCAAGTTATGTGATCGATATCCTGACCTCAGGGGGCTCTGTCTGT
CTGACGCCAGGCACACCCTGTGGAGACGACAGCCCCATGGACAGAAAAGACAAATCATCATGCAGTCCTGGGCTTTGTTCCACACAACACACAC
ACACACACACACACACACACACACACACACACACACACACACACACCTATTGTGTGGTCATTACTTTCTCTCCCAAGTTGAGTGGAACTCTGAG
CATGGTCCTTGGCCAACTATGTCAGTCACCCAGTCCCTCTCCCCTCCCGGTCTCCATCACTGTCTCTGACTCCTTCTCTCCCTCTGTTTGACAT
AGGCGAGGCTCATTAGGTGAGCCACATTGGCCTTGCACTCTCTTTTTAAAGGATTTATCTATTTTTATTTGCTGTGTATTAGTGTTTCCCTGAA
TGTATGCGTTCTGTGTCCATGTGCACACCCGGTGCCCATGGAGGCCAGAAGAGGGCATTGGATGCCCTGACGGAGTACGTGCGAGCCACCCACC
ATGTGGGTGCTGGGAACTGAACCTGGATCCTCTAGAAGAGCAGCTAGCACTCTTAACTGCTGATCTTGACCTTGAACTCTCTCTTAACCCTCCC
ATTTCTGCCTGCACAGCACTGAGGTTAGAGGCATACGCATGTGCCTGTGCCCTCAGCTGCCTACCAAGCCCCCCCAGTCCCTGGTTGGCCTTTG
GGGTTCTCCCACAATCTTTCCACCATTTCATCCCTGCCCCCCAACCCCCGCCACATTTCTTCCTTGTCTAGAAGGCCAGGATAGAAAATGGGGA
ATAGGGCTGGAGAAATGGCTCAGTGGTTAAGAGCACTGACTGTTCTTTCAAACGTCCTGAGTTCAATTCCCAGCAACCACATGATGGCTCACAA
CCATCTGTAATGGCATCTGATGCCCTCTTCTGGTGTGTCTCAACACAGCCACAATGTATTCATATATGTGTGTGTGTATATATATGTATGTATA
TATATATATATATATATATATATATATATATATATATATATATCTTTAAAAAAGAATAATTTATGATATTGAGCCGGGCATGGTGCCGCATCTC
TTTAATCCCAGCACTTGGCAGGCAGAGGCAGGCGGATTTCTGAGTTCGACACCAGCCTCGTCTACAAAGTGAGTTCCAGGACAGCCAGCGCTAT
ACAGAGAAACCCTGTCTCCAAAACAAAAACAAAAAAAAAAAGAAAGAAACAAAATGGGGAGTAGTGATGTAGGTCACATGGTAAGGTGCTTGCC
TCACTCCTACAGAGCCCTGGTTCACCCTGCTTCCACAAAACCAGATCACTCACAACGCAGAAGCAGGAGTTCAAAGCCATCTTGTAGCAAGTTC
AAGGCCAGTCTGAGATGCCAGAGACTCTCTCTTTTACAAAAGATACTGGGCATAGTGGCACATTGAGTCCAGCCTCGTTTACAGAGTGAGTTTC
AGGACAGCCAGAGCTACATTGAGAAACTCTGTCTCGAAAAACTCTTCCTTGTCCTAATTCTAGGCCACTCAGGGCTACAAGAGACCCTGTCTCC
TCCCCTAGCCGCACCCCCTGCAGAAAAAGAAAGAAAAGAAAAGCGGTAAGAACTGGAAAAAAAAATGTATGCAATTCTCAGCCTTTAATTGCCG
AAAGCACTTTATTTTCCTTTGGGTGTTCCTGGGGATTGAATTCAGGGCAGTGGACATGGCAGGCAAGCATTCTAGTGCACGCTTCACCTGCAGA
CCAATGGCTGGAGGGCTCCCTCCTGGGAGGCTCAAAGAGTGACTTCCTTCTCATTAGCCGACCAGGAGCATCTGACTAAGAGATGGGCCGCTTA
GAGCGCTGGTGCCCAGGCACCCCTGGCTCAGAGCTCCATCTGTCCAGGTCTATCTTGGGGCCAACAGGGAGCCTGCAGCTGCCTTATTGCATAT
GCACACCAGCACCTGCAAGTCTGTTTAGCTCAGGGCCTCTTCTGTGTGTCCTTTCCTGCCTTTCCCTCTGTAGGAACATTCTCTGAAACCTCAA
ATTCCAACTTTAATTCTTTAGGGATGGCACTTCCCACACCCTAGCATATCTGCTCTGTTTCCCGTCCCTTTACGTGCTTTAGAGACGCTAGCTA
CTCGTGTACCCCAGCCCAGCCTCGAACCTATGATCTTCCCGCCTCAACCTCGCAAGCGCTGAGACGCAGGCGTACATCACCATGCCCACTTCCC
ATAACTGTGTCTGCTTCTAACATCACGCTGTGAATTTTAGGATCAAGAAAACACTCTGTCTTCAGATCTGCTGCAGGAGATTTGATTCTTCCCG
CGGTCAGGTCTCCGGTGGCTGAATGAACTCATGCGCTCCTAGACTCTCACTACCATCTCTAGACTTCCGGCCAATAACGTCTCCCACTAGGATC
AGGTCGATCTAAATGTTGTACATCCTATGGATTTTGTTTTCAAACGTCCAGTGAAAATGTCGTTTTCACCTCTGAATATTCTAGGCCATTTTTC
AAGGGTCCTAACCAATGGACCCCATCCTACCTCTTAGCCCTCTTTGCCCACACACCCCAGCGCCTCCTCCTGTTTCAGTCAGGAGGCCCAATTT
CTCTTCCATGCCCTGTCACCTTCCCGTCCTCAGGATTCTGGTGGCATCTCCTTAGCCCTCTCCTTTTTGCACTCGTAGCAAATGGGACTCCCGC
CTTCCCTCTGAGAAATCCACAGAACTTTCTACGCCGCCTCTGCCTCTCTTTGGCAGCTGAACGCCTTCGACTTGAAGCTTTAACTGCTTCTTTT
GTGGGGTTCCACACCTCACTTGGGCTGTGGTCTTTTTGAAGCCGAGGAGAATACCTAGGCCCTCACACACTGACACCTCGTTGCTGCGTGGAGC
CAGGAGTGACTGCAACCCTGTCTGGCAAGGCCTTGTGGGAAGTCAGTCGATGGATGAATGTGCCAGGGGATTCTGGGATGCCCCACTTCCTGTC
TTTCGCTTCCCTGCTACATCTTGTAGTCTGGCCATGTCGTACCCCCTCACCAGCCCCAGAGCAACAGGGACAACCAACAGTGGGTTCAACTTGT
GTGCTCCAATCAGATTTCTCTCTCTCTGAGTTGACTTATTTCAGCCCTCTGTTATGGTGACACAAGGCATCAGGGGAGGGGGACAAATTAGAAC
AAAGATAATGATGATGTATGAAAATCCTGTAACTAAGCCCATTAATTTGTACTTTTAAAAATAAGATTTATTTATTTCATATATGTGAATACAC
TGTTGCTGTCTTCAGACACACCAGAAGAGGTTGTCAATCCCATTACAGATGGTCGTGAGCCACCATGTGGTTGTTGGGAATTGAACTCAGGACC
TCTCGAAGAGCAGTCAGTGCTCTTAACCTCTGAGTCCTAATTTGTACTTTTTTTAAATTTTTATATCCTGTTCTTCACAGTAGCCTCTCCTGGA
AGCACCAACCTGAGTTCTTCTTTAATTCTTTCTCAAACAGCATATCTAAATTTTGCGATCATAAGAACAAGCCAGGAGAGTACATATTCTATGC
AGAAAATGATGACGCCCAGTTCACCAAAATGTTCACGCTGAATATAACAAGTAAGTTGAATCTCATCTTGTCTATTTTATTCCTCCATTAATTT
TTAATTAAGAAGAGCCAAACACAGCTTAGGAGGGACGATATCAAACTAATTTAAAAAAAAAAAAAAAGGATTATTTGCATGTATATGAGTGTTT
TGTAAGTGGTATGTATGTCTCTGGCCCACATGCATGCAGTGCCCACAAACCCCACAAGAGGGCGTCTGATCCCCTTGGAACTTGAGTTACAGAT
GGTTGTGAGCCATGATGTGGGTGTTGGGAACCCAGTTCCTCTGCAATCACAACCAGTGATCCTAGCCTCTGAGCCATCTCTCCAGCCTCTGAAA
CTGCTCTTGTAAGCGCCATCCTTCTTGTGTGTTGGCTGTAGTTATATATGTCCCTTTCTATACTTGTTCTCTCAGCTAGTACATGTTCCATCTC
CGATGTCCTCTGACTTTACTGCCTTCGCCCTATGAAAGGAACACTAAGGAAACGCTATACAGTCACCTGCTACTTATTTCTTTTAATGATTTTC
GTCTTCGTCTATGCAAAATAAGAAAAAGACACTATATTTCATTAAGGTTTTTGCAGATTTTTTTTACCTGCATTTTTTTTTTTTACTTGCATGT
ATGTCTGTGTGAAGGTGCCAGATATTGGAGTTACAGACAGCTATAAGCTGCCATGTCGATGCTGGGAATTGAACCCAGGTCCTCTGGGTGGTAT
TTTTAAAGCTAAGTGTTACATTGACGTTTGCAGATAGATGGAGGTTCTACACTTCAGCTCCCTCTAGGAGGTGAGTCATAGAGACGTGTTCCCT
CCACCAGTCAAGAAAGTTTTGTGACAGAGGAGGAAGGGACCAAACAAACTTTGAGGCATCCTAAATAAGGGGTTGTTCCAGGGGTACATAGCAC
CTGGGCTAAGAAGCAGCCTAAGTTGAAGCTAGCTCTGAACTGATTCTAGAAAAAAAAAAAAAAAATCTATCTTCCATGAGCTTACTGCGCGCTA
CTTTCTACAGAGAAACCTCAAGTGCTAGCAAATGCCTCAGCCAGCCAGGCGTCCTGTTCCTCTGATCGCTACCCGCTACCCTCTTGGACCTGGA
AGAAGTGTTCGGACAAATCTCCCAAGTAATGAGAACCCTGACGTCTCTTGAAGTGGATGAGCCAAGCCGGGCAGTGGTGGCGCATGCCTGTGAT
CCCACCACTCGGGAGGCTGAGGCAGGACGATCTTAGAGTTTGAGGCCAGCCTTGGGGAACACAGTGAGGCCCTTTCTCAAAAAACAAGAAGGAA
AAAAAGGAAAGAAGGGAAGGAAGGAGGGAGCGAGGGAGGGCAAGAGGGGCAAGGGAAAGGAGAAAGAAAAAGAAAGGAAGAAGAAAAGTGAGAA
TAAGGCATAAGATTCTATCACCTGTTGCCATGGATACAGCTTAAGAATGACATTGAAAATGCTTACCCTACAGCCACAGCTTGTTTTCTTTGGG
AACCATCTGTAGAAACCCTGCCTTCCTGTCCTACCATGACCCCACCCATCTAACTTCCTGTCCTACCATGACCCCGCCCATCTAACAATCCCTT
TCCACCATTTTTTTTTTCTTTTCAGTTGCACGGAGGAAATGGGAGAAGGAGTTTGGAATAAAAAGGCTAACAGAAAAGTGTTTGGCCAGTGGGT
GTCGAGCAGTACTCTAAATATGAGTGAGGCCGGGAAACGGCTTCTGGTCAAATGCTGTGCGTACAATTCTATGGGCACGTCTTGCGAAACCATC
TTTTTAAACTCACCAGGTGTGCAGCCATTTGCTGTTGTGATAAACACACCCTGTCCCCAGAAATTCCCCCACCTGAAGTTGGGCATTGTGGCTG
GCCTTTGCTGGGGAATTCTTATGAGATTGCCCAGTTCTGAGTGAGCATAAGGTCTTTTTGAGTCAAGCAACAATCCCCAGCAGACTGTAATTTG
ACGTTTCAGCACCCAGTTATCCTTAGGCTGTGTGACTCACAGGTCACGGAGCTGTGGAGTCTGGGAAATCCATCGTGTGAAGATTAGCCTCCAG
AGGTGCAGGAAAGCAGACTTAGTGATCTGGAATTTCTGTCCTTTTCCATTCTGTCAGTTCCCTTATTTACTTTGAGCCGGCTAGTCTAGATTTT
GGAGTTGTGTTTAGAGTCTTTCTAAAATGACACTAACAGTTTAGGGTCACCAGATAGTATTGATTAACTCCAGGGACAAAAGCAAGAGGCTCCT
AAGTTCACGTTCAGCCTGGGCTCCATAGTAAGACGCTGTCTCAGAAAAATGCCAAACAAAGCAAGCACGTAAGTCAAAATTGTGGCTTTGGTCT
CAGTAGTTAAGAACACAGGCTGCTCTTGCAGAGGACCTGGGTTTAATTCCCAGTACCCACATGGTGGCTCATAGCCATCTGTAGCTTTAGCTCC
AGCTCTGGGGGGTGGGGGGGACCTTAACGCCCTCTTCTGGCCTCTGAGGGCTCCAGGCATGTACATGGTACACAAACACACATGCAGGCTAGCA
GTCATACACATAACTTCTCTACATGTGTGTGACGAACCTTGATAACAAATACACAGACTTCTTTTTACCTGACACGGGCTCTGTAAAAGGTTTA
TATGGGCTTTATTCACGTGACATCATGGTCCAGTTTTTAAGATGGGTTCAGAATCGTTTCTGATTTTTCTGGGGACTCATTGTTTACAGAAACA
AACCAACTTTCTGTGGGGCACCCAGACAGAACACACAGATTGCTCATAAGGCCTGAAGATGGTGGTTACTTGAACAGGATGTGAGAGACTCTAA
CAAGAGACTTAAACAGGCCTGCCTGTTTTATCTCGCCCCCGCCTAGTGATTGCTTCTCTTGCAGGCCCCTTCCCTTTCATCCAAGACAACATCT
CCTTCTATGCGACCATTGGGCTCTGTCTCCCCTTCATTGTTGTTCTCATTGTGTTCATCTGCCACAAATACAAAAAGGTAAAAACCGGTTGGCG
AAGGCTCATCCCTGTCGTGACTCTGCAGCGCTGCTCACCCGCTGTTCATCTCTCAAGCAATTTAGGTACGAGAGTCAGCTGCAGATGATCCAGG
TGACTGGCCCCCTGGATAACGAGTACTTCTACGTTGACTTCAGGGACTATGAATATGACCTTAAGTGGGAGTTCCCGAGAGAGAACTTAGAGTT
TGGTAAGGATTGACTGTTCAGAGCTTCGGTGGCATTGCCTCTGCCTTGCAAACATTTTTTTAACCCGCACTTACTCCATCTTTACAGGGAAGGT
CCTGGGGTCTGGCGCTTTCGGGAGGGTGATGAACGCCACGGCCTATGGCATTAGTAAAACGGGAGTCTCAATTCAGGTGGCGGTGAAGATGCTA
AAAGCTACCAAAAGGTGGGGTTATCTCAGCAAAGTCCCTCAGAGAACGAGTCCAGCTGCCTCCCCTCCCCTTCTTCTGCCCTCTGCATTATTCT
GTCTGCTAAGCACTGAGGGTTAACGAGAAGCAGGCAGGGGCAGTCGTGAGCAAAGACTGCAAGCAGGGAAGGCAAGCCCCCACCCCTTCCTCTT
TCTCATTGTTCAGTTGGTATCAGCTTCTGTGTTACAGTTTGACACAGATTCTGGGTAGAAAACACACCGGGGACTTCTCACTTCCCACTCTGTC
CCATCCTCTCCCATCTGGCATCCACCCTCGAGCAGGGCCACCCTCAGCCGATTCTTAACCCAGCGCAGAGGATTGAATCCAGGATTTTGTGAAC
GCTAGGCAAGTCCTCTACCTCAGAAATACATCCCCAGCCTGTCATTCTATAACACGATGTCTTGCTATATAGGTCAGGCAGGCCTTGAACTCAC
AACTTCAAGAATGCTGAGATGTATCATCCCCTCGGGACTGATGGCCCATTCTTTTGCTGAGGTGTTCCCTGTAAGGATAAAGGGAGCCCCTCTC
TCCAAACACACACACACACACACACACACACACACACACACACACACACACACACACACTACAGAGAGAGTGACAGAGACAGAGAGAAACAGAC
AGAGACAGAGAGAGACGCAGAGAGAGACAGAGACAGAGAGTCACTGAAGTGCATTGCAGTACGCAGACCAAAAGGAGAAATGGCACACAGGACA
TCATGGACAGAAATCTCACAGTCACCTCCTACCCAGGTGACTGATGATGCTATACATCCTGGGTTACAGAAAGAAACGGAAGCTGGAACTGTTA
GAGGATAGCTTTAGGATACATACATTTGCCGGGCGGTCGTCGTGCACACCTTTAATCCCAGCACTTGGGAGGCAGAGGCAGGTGGATTTCTCAG
TTCGAGGCCAGCCTGGTCTACAGAGTGAGTTCCAGGACAGCCAGGACTATACTGAGAAACCCTGTCTCAAAAAAACAAAACAAAACAAAACAAA
ACAGGATACATACATTTTCTGTAGATCAAATTCAGGATGTGAAAAGGGATAGACTTTCCCCTCTGATATCTGCAAGGTACAAAACCTTGAAGCA
ATGTCAGAACACGATCACTTGTTACATCCGGAGAACAGATATGAACGTTTCTTTTTACACTGTTTTTCTTCACCCGTACATCCTAGTTCCACAG
AATAAGGAGTGTCGTCGCTGTGGTATTCCCATATGTATACTTTGGTCCAGTTCACTAGCACCGTTCCCCCTCCCCTCGTCAATTCTCTTCCTGT
CGGTCCATTTCTTCTTCTCAAATAGCTCCCCCCTCTACTTCCGTGCCTTTTCCTTTTAATCTCGATCCTACATATGAGAGATGATGCACTGCTT
GTCTTCCTGAGTTTGGCTTTTTTACTTAACATCCCAGTGTCTGGTTCCATCCATCTTCCTGCAGGTGGCCCATCCCGTTCTTCGCAGCCAAATA
CACCACTTCCTATGGATCGTATTTCCAACATCCATCCATCCATCTGTCGCAGGCCCCTAGGCTCGTTAGATAGCATGTTGATTGTAATAGTGCC
AACATCGACTTCCTGTGTGGAGTTCCTTATGCCTCCACTCGCTCCACGCACTCACCAAGGACCCCCCTCTATAACCCAGCTTCATCTCCTGTAC
CAATAGCTCTCATAACTGGTTCCCTAAGCCTTGGCTTTGTGTGCCCAAATACTTGTATCTCAGCACTGTTCATCCAGGCTGAAGTCCTCCAACC
CTGGCCTCCATCTCTACTCCAGCCTGGTAATTACTGGAACGTTTCAGTGAGACTACACATTCCACACAACCATAGAAACAAGGAAGCGGGTTCC
ATAGCTGGCTCTCTCTCAGCCTTCCCCAACAGGTTTCCTGCACACTGTCTGCTGGGCCCATCCTCATCTCTCCGTGGTCTCTGTGGTGGTCCAG
TTCTGTCTCTCCTAGAGACTCTTAATGGTAGCTTTTAAAAAGCATCCCACCGATTCTGCTAGGGGCTATGTGCTGAGGTGGGGCATGGCCACGA
TGCAGAACACCTTCTTGCTTGGCACTGCCCTAAGCATTGACAATGGCTTTCTACTGCTCTGAAAATGGTTCCCACACGGGCTGGAGAGATCCCT
CAGTCAGTGGTTAAGAGCACTGACTGCTCTTCTGAAGGTCCTGAGTTCAAATCCCAGCAACCACATGGTGGCTCACAACCATCTATAATGAGAT
CTGAATCCCTCTCCTGGTGCGTCTGAAGACAGCTACAGTGTACTCACATATAATAAATAAATAAATCTTAAAAGAAGAAAGAAAAAAGAAAGAA
AAGAAAATGGTTCCCACAACTTTGACCAGTGCAGCCGTGGCTCAGCCTTTCCGCCTCATTCTTAACCTTCTTTCGCTTCCCCAGAGTGCCTCGT
TTCCCTGCCCACAGGCTTTGCGCAGGTGAATCCTTCCACCTGCTCTTCCCCTTTTGCTGTAGGAAGCCACTTACCTCTCAAATGTCAGCTTTGA
GATCACTTCCTCAGGGACACCTCCTTTCACGCCATGGACAAGATCAAGTCCCTGGGGACCCTGGGTGTTCTAAGTGGTACAAGACATACTCAGC
ATGCTTGAGGCCCTGAGACGAATTCTAACATTAAAAATATAGTACGTCCTAAGAACACTATGTAGACTTCTCCCAGAATCGGAGACTCTAACAC
AGTTGCGTTTAGGCATTCAGTTAGGTTCTAAGCCCTCTGAAAGCGGCATGTGGCTTTTCCCGTGCTGTTGTTACTTAATACTGTGTATTCTGCT
GCCTCCGCCTGCGGTGGTCTTGTGTGACTCCTTTCGGTTCCCCACTGAGGGAGAACTACAGGGTCCTGCTTTTTCGGGCTGTCTGGTGTCCCCC
CTAGAGTTCCCCCACAGGACTTTCACTGTCTCTCTCTCATCTTGTGTGGTCATCCCTCAGTTTCTGCAGGAAGTTGGTTGCACAGTCCCCTTTG
CAGACGCCAGGGTCCCAGACGATCAGGTCCCTTCTATAACACAACAGTATGTGCATAGAACCAGGAGTCCCTGGAACAGAGACCCCAAAGAAAG
GTGTTACCCTTGTCCCCGAGGCCCCAAAACCCACGGATGCAAGGGTGCCCCAGGCAGCTATGCTAGCCTCAGTGACCAGAGCCCTGGGAGTAAC
AACCCATGCTTGCATCCTAGCAAGATCGTGGACACAGTTTGCTCCACTGCAGAGATTGCATTGGTTCATGTGTTGTCTTCTTTAACTTTCCCCC
ATTGACCACACTTACCAATGGAAGTTTCTAGACCTGAGGAGCAACTTCATGTTGGAGACATCTGTGCTTGCTGACCCTCCTCCTTCTGGTCTTC
CTATCAGCTGAATAATCTGATTTTTTTTTAACTACGAAAATCACGTTCTGTTTATCGTGTTGTCTACCTGCTTTGAGAGCTGGGTGGAAGGACT
TTTATTTTTCTCAGGGAAAGTCAAGTAGAAATGTATATTCTTGCTGGGCATGGTGGTGCATAATTTGATCCCAGCTCTCAGGAAGTAGACGTAG
GCAGGACTCAGTGAATTCGAGGCCAGCCTAATCTGCACGGTGAGTTCCACGCCAGCCAGCGCTATATAGTGAGACCCCGTCTCGAGGAAGGAAG
AGATTATACATTCATATAGTTAATATCTATAACTCACGTTTTAAAACGCTGTCTTAAAACACAGAACATTATTGTCTAAATTTATGTGTTATTT
TTGTGTCAGTTTTTCTCTTCCAGTTTTGAATGTGTGTAAGTTGTGATCTGAGCCAAGGAGAGCCTTGGTTTCTAAAACAGCATACAGTTCCCTG
AGGTCAATCCTGTTTCTGCTCCTCCACCAACAGAGAAAGCTGACAGCTGTGAAAAAGAAGCTCTCATGTCGGAGCTCAAAATGATGACCCACCT
GGGACACCATGACAACATCGTGAATCTGCTGGGGGCATGCACACTCTCAGGTAATGTCTACACATCTGCATATCGAAGCCCTGTTCCACACTAC
TGCAGCCCAAGGGAGCGAGGCCTGCGCTCTTTCCTCAGCCCCAGTTTGTAGACGATGGGTCTGTTTCTTCCTTCCAGCCCCATGCTCCCAGAAA
TAAGACTCAGACTCAAAATATATTTACAAATACCTTGACCATAGAGCTAGGCTCTCCTTTGACTAGATAAAAACTTAATATAACCCATTTATTC
TAATCTACATTCTGCCACCTGTGCTCGGGTGCCATGAGCCTCTCTCGTCTCGTTTTCTCACGTCTTCCTGGGTGAATCTCCCAAGCCTGGCTCT
ATCCCAGAATCCTTTCTGTCTCCCAGATGTCCTACCTCTTATTTCATTCCTAAACCATAGGCCATCAGACTTATATGGGCATAAGCTATTCTCT
TCACACCAGTTCAAGTGGAATTAGACGGTGTCCCCTCTGCAGAGCACATGACCCCGTCTTTCCCCTACTCTATCTGTCACAGACTCCTAACCGC
CTGAGAGAAAGCGGCCACCCTGCACATTCATGTTGTGGGGTCTGAGCTCCTCCCTAGACTGTGAAGCTGGCCTAAATGCAGTAGTGACATCAGC
TTCAGAACTCAGCACACACATACTGATATGTTGATGATGGTGAGCTCAGACGTTTAGAACTGGAGAGAAAGGCTAGAAGACAGATTCTTGTATA
CAGTGCTGAGATCGAGTGTAGTCTAATGTACGAGCTCATTTAAAGCACCTTCTGAGGGCAAGCAGAGCAGGAAAGGCCCAGGTCCTGTTGCGCT
ACAGTGTGATTTTGGAGGAAAGACCTGACCTGTGTCTAAACCTGTAACTACAATTGTAACTGTTTCAGGGCCAGTGTCTGCAGTCTGTCTAACC
TTGTAACTACAATTGTAACTGTTTCAGGGCCAGTGTACTTGATTTTTGAATATTGTTGCTATGGTGACCTCCTCAACTACCTAAGAAGTAAAAG
AGAGAAGTTTCACAGGACATGGACAGAGATTTTTAAGGAACATAATTTCAGTTTTTACCCTACTTTCCAGGCACATTCAAATTCCAGGTAAGAG
CTCCATCAGAGGTTTATGGCTGTAAACCCCGGGACAGACAGAATGACCACTGCACACACCTACCTGCAACCCCACCCGGGCAACATTCGGAGTT
GCTGTGTGTGGCCTGAGACCTAAGGCAGCTATTTGTTTATGCTGGGAATTTCTGTCCCATTATTTCTTATTTTACTTAATAAAGTTTCAATTAC
CCACCAGAAGACATGGTTCAAAGATCCTAAATAGAAAAGTCCAGAAATAAACAATCCTTAAGTTTTAAGTACCTTTACAAACAGCTTATTAATT
AACATTATACTATCTTATCATTCGTTATTATTGTTCGTGTGTTACTATGCCCAATTTATTTGTACTTTACTTACTATTATATTTCATTTATTAT
TTATTATTGTGTGAGTTTGTAGTGCTAGGGGGCAAACCCAGGGCTTTATGAATGCTTGACGCATCCTCCCTCTCTGAGCTACATGGGTAGTCTG
CCATGCATGCTCTAGAGGAACTATCCTAACACAGACGTGCGTAGCCAATAATTAGATCCTTTGGTTCATTTTTGGTCACTGGGCTATTAAATAA
ACTAAAAGCATTAAGCCTTTTTTCAAAGGCAAGGGGAGTTGAAAACTGGGATAAACTCTTATGGTGGGAAATACTGAGTCCTGACCTATTTGTA
GTGGTGCGGGCCTCTCTTTGATGCGCTTGAAAAGGTAATCTGGCCAGACAGTGGTGGTGGTGGCATGGTGCACGCCTTTAGTCCTAGCACTCGG
GAGGCAGAGACAGATCTCTGTGAGTTCAAGGCCAGCCTGGTCTACTGAGAGTTCCAGAGCAGCCAGGGATAGACAGAGAAACCCTGTCTCTAAA
AACCAGACCAAAACAAAACAAAAACAAACAAACGAAAGAATGAAAGAAAGAAAGAAAGAAAGAAAGAAAGAAAGAAAGAAAGAACAAGCAAAAG
GAAAGACAGAATCTGTTGCACCTATTTGCTTATCCCTCAGAGGAGGGGAACGTGGTTCACAGACGCTGGCTTGAAAAGATGGCGCTGGAAATGT
ACACCTTCCCACGAGGCAGAAACCTACACATCAAACACATTGATGAAAAGAATATCAAAACTCTAAATTTGTAAGCTCTGTGTTGCATGAACTC
TGAAAGGTGAAATGGCCTGTGTCTGGAGCAGCAGGATGCCCTGTAAATTGGTTCCTCTCTTTATAAACTTGAGGACATAGTAATCATTTACTGC
CCCAGTTTCTGTTTTATGTACCCAGCTTACAGCGTGACAGTGACTGAATCCCAGCTCTTATCCAGCTTCCCTCGGCAAATTCCAACACACAGAT
GTTTTTAATGTACACGGGGTACCGTTGACAAGCCAGCATCGTGCTTTCTGTTTAAAAGCCCATTTGTAACTGAAATCTGTTCCTCTGATGAATT
GTCCTTTTTGTAATAAAGTCTCTGCTCAGAGGAACAATCTAAATAACAAAGGAACAAATTTATTTTTAATAGCATGCCTGGTTCACGAGAAGTT
CAGTTACACCCGCCCTTGGATCAGCTCTCACGGTTCAATGGGAATTCAATTCATTCTGAAGGTAATGTTTTACTTTTCATTGGTATTTTACACT
CACTGAAATGAATCCAAGAGTCCCATGCCAGCTATTTTTAAACTTTTTAAAAAAATTATGACTGTATGTGTGTATGTGCATACACGTGTGCATG
CATGGTGTGTATATGTGTGTGCTGTGTGTATGTGTGTGTTTTATGTGTATATGTTTATGTGTGCTGTGTGTGTATCTGTGCATGCTATATATAC
ATATACATACATGTATATATATGTATATATATGTATTGTATGCATGTATGTGTGCTGTGAATGTTTGTATTTGTGTACATGTGTGTGTCGTATG
TGTGTGTATGTGTGTCGAGTGTGTGTATATATATATGATGAATGTGTATGTAAGGTTTGTGTTTATGTGTATATGTGTTTTTGTGTAGGGTGTG
TGTTTATATGTATGTATGTGTGTATATGTTTGTATGTGTGTATATCTGTGGAGGATATGTGTATGTAAGTATGTGTGTGTGTTTGTGTATAGAG
TATGTGTTTATGTGTATGCGTGTGTTGTATGTGTGTATGTGTGTGTTTATGTGTATGTGTGTATATATGTGTGGAATATGTGTATGTATGTGTG
TGTTTGTGTGTAGGGTATGTATGTATGTATGTTTATGTGTATGTGTGTGGTATGTATATATGTATATGTGTATGTTTGTAGGTGTATATATGTA
TGTGTGATATGTGTATGTATGTGTGTGTTTGTATGTGTGATATATATATACACACATATACATATATATGTATATGTGGGTTTGTATGTGTGTA
TATGTGTGTGATATGTATATGATGTGTGTGTATTTGTATGTGTGTGGTGTATATATATATGTATATATGTTTTGTGATATGTGTATGTATGTGT
GTGTTTGTATATTGTGATGTTTAAAGATATATATGTATATATGTATACGTGTGTGTCTGTGTGTGGTGTCTATATATATATATATTTATTTATA
TGTATATGTGTGTCTTTGTATGTGTGTCATATCTGTACATATGTATGAATCTGTCTCCTATATATATGTATGTGTGTATATATATATATCTGTC
TGTGTGTGTGTGTGTGTGTTTGTATGTGTGTGGGGTATAAGTATATATGTGTGTATGTGTTTGTATGTGTATGAAGTATATATATGTACATGTG
TGTATTTGTATATGTATGGTATATGTCTGTGTATATATATGTGTGTGTGTGTGTGTGTGTGTGTGTGTGTGTGTCTGTTTGTATGTGTATATGT
GTGTGTGCTTGTGTGTCGGCATGATCGTGCCGTGGCACCCATGAAGGTCAACTAGTAAGAGCTAGTACCAGGGCTCAGTACCAGGGTGTCAGCC
TGATGGTAGCTTTTCTCCAGTGAGCCATGCCACCAGAGTGGAAGCAGTGTGTCTTTGCTCTGGTCAAAGGCAGACTCTGTCAGTGCACACTTTC
TCGGAAAGTCAAAAGCTGATAGTTGTGAACTCTGTGTCCATGGTCAACAACAGGCACCTGAACTGTTTTCTTTGTTCGGAATATTCTTTATAAA
GTCATAAGCTAATTGGAGCCAGCTTGTACCTGGTAAGACTGTGAGCTCTAGAATCAGAGTTTGGTTATGTTCCAACTCTTTTCCTTTCGAAACA
GTTGGCTCCAGACGACAGTCCCCACTTCTCACTTATAAAATTGGTCCAATTCTACTTTCCTCATAGGACTGTTAGGAGGCATACACACAAGCTT
GTGAAGTATAAAGTACAGTCCATGAAATGTGGTACACAGTTCGTCAGTCTTTGCTTTTTTCTACTGACAGATTTACCTAGCATGAAGGGAACGA
GGGATAATGTTTGGGAGTTTTGCTTACCCTCTCTGTCTGTGCACCACCTGGGTGCAGTGCCCTTGGAGACCCAGAAAAGGGCACTCGATGCTCT
GAGACTGGAATTGTGAGCCACCATATGAGTTCAGGGAACAGAACCCAGGTCCTCTGGGAGAGCAGCCAGTGCTCTTGAATCACTGAGTCCCAAG
AAGGATTCATAATTAGTATTCCTTGTCTTAGCTCAGATGGCTTTAACAGATTATTAAAGACTGGGTATCATAAGCTACAGTATTTCTCACAGTT
TCAGAGGCTACTGCTTTCAAAACTGAGGTGCAGCAGACACTTGCTTCTCACATGCACATGCTGATCTTTCTGGGTCCTCAGGCCTCACACAGAG
GCAAGAGTACACGCTTTCTTCTCTTTAAAAAACTTTAAAAAAAAAGTATTTAGTGTGTGTCTTGTGGGGGAGCAACTTACAGGAATCCATTCTC
TCCTTCCACCATGTGGGTTCCAGGGATTAAACTCAGGTCTTTCGCCTCTGTAGAAACACCTTTACCTGACAAGCCATCTTGCCCATTCTCCTCT
TCCTATTTTATAGAGTTCATGGTCCCATTTTTATAACCTGGCCACTTCACAAACACCCTACTCCTGATACCATTGCTTGAGAGTTCGTTTGCCT
GTATGTGTGTATGCATGTATGTATACACGTATGTATGTATGTATGTATGTATGTATGTATGTATGTATGTATGTACGCACGCATATATGTATGC
ATGCATGCATGCATGTATGTATGTATATGTGTATACATGCATGTATGTACATATGTATGTCCACTGTATGTGCCTGGTACCTTTGGAGGCCAAA
ACATAGAGTCACATTCTCTCCAGCTACCATTGTTACAGACAGTTCTGAGCCATCATATGGGTGCTCCGAACCAAGCCTGGGTTCTCTGTAAGAT
CATCCAGTGTTCTTAGCTATGGACCCGTCTCTCCAGCCCCTGGGGATTTGAACAGGGCTTCATTCCAGAAAACAGAATGAGTGTTCAAGTAGAC
TAAGAGGAGACACGTTTATAGAGACAAAAAAATGACTAAGGGCAGGAAAAAAAGGGACGCAGAGATCAGGACAGGAATCACTCTGCACAACTTT
AACCCAAGCATCCAGGCGGCAGAGTGTTAGTCTGTGAGTTCAAGGTCAGCTAAGTCTACATAGTACATTTCGGGACAGCCAGGGCCACATGTTG
CTTATGAGGACAGGCTGATCATTCCAATCATTTCCCCGGGTTTGGAGTCAGGAAATGGACAAGGTGGCTAGTAGCTTAGCCCCAGGCTCCCCAG
GAGAAGCTCTTCTGTAAGGTTTGAAGCAGAGGCCATTCCATCTTCATGCCCAGGAACCTGGCCTGCGCAGAAGGTTTGATGGTTGCTTCGGATT
TCTCCGAAGGTCAAATAATCCTTTCTCTTTGTATGGCGGACCTTTTGTCTGGGTGACGCCATTCGATTTGGTCTATTGAGTCTAGAGCAGGAGA
TCAGATCAAACGCCTCTTATAAATATCTCCACACTGAGGTCGCCTTAGCTGCCTCTCACAGAGGTCTGATAGGGTACTGCCTAGTGTCTGGGGA
AAGTGACAAGTGGACGTCTTTTTACAGATGAGATTGAATATGAAAACCAGAAGAGGCTGGCAGAAGAAGAGGAGGAAGATTTGAACGTGCTGAC
GTTTGAAGACCTCCTTTGCTTTGCGTACCAAGTGGCCAAAGGCATGGAATTCCTGGAGTTCAAGTCGGTAAGTACCTTTTAAAGAGAAAACCGA
CAGAAGGCGCAAACACAGAGCCGCAGAAAGGAATGCATGCGTGCAGCTTTGAATGAGCCGTGTGTCTACGTTTGTTCCATGGTCATTTTAACAT
GCCTCCCTTGCTGTCTGCCCTGCTTCCCCTAATTCTTTTCATACCTGCAGCACGAAGGCCCAGAGTCACTGGTTAGAGCCAACGTCCCGTGGGA
TGTCTAGGGAAGTCCCAGAAAAGCCATACTGAAAGAAGCACAGTATCACAGTATCGTCATCCCTGCCAATACAGGGATTCATTCGGGTTGTTCC
CTGCAGTCGGGGGCATGAGCAGAGGCCCAAAGTTCACCAGCTCTGCCACCCCAGCCAGCCAGCCTGAACAGATTCAGCCTAGGCTAGCCACAGC
GCTGTGGAACCTGTCTCTTCTCAGTGGCTGGCCCTGTATTCACAAGTGCTGCAGCAGGAAGACTGCGGGACCCACCCCAAACCATGACCTCCCC
GTGCTGCGCTAGGCAATGCTGCTTCTCCACCCGCTGTCCCCTGTCATTCCACACTATAAGCCACCAAAGCCTCAAATCTTTATGTCTGCTGAAG
TAACATGGTAGTAAACAGATTAGCGGGGGTGGGGTGGGGGGGGGGAGACAACATCTTTAGCTGTGTACGCACCTACACAAACTATTGAAACACA
AGGAAGGGCTAGGTTGGTGTATCTTACTAAGTTTCTCCATAGGAAAAGATGTCTGTGTTTCAGAGGGGTAGTATGTTAATTAGGGCTCCCTAAA
GAAACAGAACTGATAGAATTAAAAATAATACATATATATGTATTATTTTTAATTATAAAGTTATATATAGGGCTGGAGAGATGGCTCAGTAGTT
AAGAGCACTGACTGCTCTTCCAGAGGTCCTGAGTTCAATTCTCAGCAACCACACGGTGGGTCACAACCATCTGTAATGGGATCTGATGACCACT
TCTGCTGTTTCAAGAGAGCAACAGAGTACTCATATAAAATAAATAAATAAGTAAAAATTTAAAAAGTTATATATATATAAGTATATATACATAT
AAAATTATATATTATTTTACTCTCTATGTAAAAATATTTTATGCACAGACATACACACATACACACACGCATATATGGGGGATTTGTTAGAGCG
GCTTACAGGCAGCAGTTCATGTAATCCAACAAGGTGCACGAGACTGGATGTCTCAGAAATCCCCATCTGGAGTCCCGGGGAAGTCTTAGAAAGC
AGGTGGTCCTGGGTCTACATTGGGATCCTGAAGAAGTCAGTGAGAAAGCAACCACAGCAGCAGAGCAGATGGACTTGCCAGTAAGAGTGAAGGC
AAGCAGGCAAAATGCAAACGCTTCCTCCTTCATACCCTCTATGGCTGCCACCAGAAGGCGTGGCCCAGGTGTTAGGCAGGTCTTCTGACCTCAG
AATCCAGAAATTCTCTGTCCCCCGTGCTGCAGAGCCGCAATGACCAGTTTAGATAATCCAGCACAGTTTGTTCATGTTCTCTAATTACAGCAGG
CCTTTTTATTTTTACTCTTAGGTGTGTTATGACATGGACTTTTGAAGAGCATCCAGTTTTTTTTTTTTAAAGATTTATTTATTATTATATCTAA
GTACACTGTAACTGTCTTCAGACGCACCAGAAGAGGGCATCAGATCTCATGGATGAGATGTGAGCCACCATGTGGTTGTTGGGATTTGAACTCA
GGATCTTTGCAAGAGCAGTCAGTGCTCTTAACCACTGAACCATCTCTCCAGCCCCGGGCATCCAGTTTTTAGACGGTTTGTCTTCACTGCAGCC
TTCCGCTCCAATCATGCCAAGCACTGTGCTCTTCCCCACTCTGTGACTGTGTCACACATTTGTGTGGCTCCATGCGACACTGTGCTGGGGCAGA
AAACCAATGAGGATGTGACTTCAGGGCCCACAGGTTTTATGTTTCTAGGAGAAAGACAAGCAGACCTATACATCAGATCAGTGTCTTGTGATTG
ATACTGTCACTAAAGTTAGCATGGCTACCCCAAGAAAAAAGGGGTGAGGAACAGGCAAGAGAGCAAGAGAGCCTCCCTTGGATGGAATCCAGAG
GGAGAGGGACTAGTCAAAGCCAGTCCAGGCTGGGCAGGCTTCTTGCTGGTCAGAAGGAGCCTTCCCAGAGCAGCTGGGAAGAATACGGGCAGGG
CAGGTAGTCAGGAGGACATGATGGGGATGGGGACAGACATGTAGGAGATGCCACATGAAGAAAGCCAGACTTGGAGAAGGAGGGCAGGAGTCAC
GGCACAGACATTCCTGTTTTTATAGGCTTTGTTCTTTTAAAGGCTGAGGAGAGCGTAGTGGACCCCCATGGGACACCATGTGCTCTCTTACAGA
GTTCATTCCCTTCAGAGACTCTGGGCTCTACTCTCATGTACTCCTCGAAACTCATCGTTATCTTCTGTCTGTCTGTCTGCCCCCTGTACCCTGT
ATGTGCATGTGAAAGCAAGTGTGTGTGTGTGTGTGTGTGTGTGTGTGTGTGTGTGTGTGTGTGTGTATGAGAATGTGGGGCCCATGTACCGTAG
TGTGTGTGCAGAAGTCAGAGGACAAACTGTGGGAGTTAATTCTTCCACTGTGGGACCCAGGAATCGAACTCAGGACATCAGGGTTACATGGTTA
ATGCGTTTTCTCACTGAGCTCTCATCTCTTTGGTCCCCCCAAACTCACTTCTTCTGAATGCTTTCTTCGATCTTAATCTATTTATTCATTTAGA
GACACGACCTCTATGTGTAGTCCAGGCTGGCCTCAAACTTAGAAATCCTCCTGCCTCAGCCTCTCCAGTGCTGCGATTATGGATCTGCACCACT
AGGCCTGTCAGATATGATGCACTTTTATTCTGAAAGCCAGTTCTTTATCCAAAAGGGATTCTGTTCTCGCACGCACACACGCACGCACAGGAAG
CTGGCTGTTAGAAGCGTCTCTGGAGCTAGGCGAAGGCCCCCTTTGGCACTTCTTTCGCTCATAGCTCCAGGGATGCCTCAGATGACTGCAGCTG
AGTTAACTTAGGACTCAGCCAGTGTTCTCGGGATGTATGACAGTCATGGGTCTCTGTCCTCTGTAACTGTTGTGAGTCTCTCTCTGAAGGTGTA
AGTCACAGACCATGTCATTCCAGCCTCTCTCTTCCAGTGTGTCCACAGAGACCTGGCAGCCAGGAATGTGTTGGTCACCCACGGGAAGGTGGTG
AAGATCTGTGACTTTGGACTGGCCCCAGACATCCTGAGCGACTCCACCTACGTCGTCACGGGCAACGTGACCCGTTTGCTTTTTGTCACGAGTA
CACGAAACACCTGCACTCTCTGTGATGCGTTTTTGATTTATGGTGACACCAAGGGGAGAAAAAAAAAACTCCCAGTGCTTCACTTCTTTAGGCT
CAAAACATTCTTTTGTGCTCACTAACAGCTAACCAAAATAGCCACGCATCTCTGGCGGGCTCGCCACTCAGAACAGATCCATTAGCCGTTGCCA
TGAGATCTTCACCTGAAAAAAAAAATGCCCTTACTCAGACCTGTTTCGGTTTCCTTAGCTATAAAGTGGCTTTATATGTGTTTTATGTTTCCGA
TCATGAGACAAGCTAGCATGTATAAAAAGCTTTTTGTTTTTTTTTTTTCTGAACACGCAGCACACACGTATTCACTATAAAGTTTAAAGGTGCT
GTAGTTAACTGGTAGCGTTTGTTACCCTGTTACTATATGAAGAGTTCAGGAAGACTCCAAATACAACATAACTGACATGTGTTCGTTCAAACAG
AATGTGAGGGGGCCAGAGAGATGGCTCAGTGGTTAAGAGCATAGAGTGCTTTTCCAGGGGACCCAAGTTTGATACCCAGCACCCACGGCAGGAT
GCTCGCAACCATCTATAACTCCAGCTCCAAGGAATCTAGTGCCTCTGGCCTCTTTGGACATGTGTGCTCATGTGCACATACTTACACATAAGTC
AAAATCAATTCAAAGTATGATGAGGCAAAAAATGAATGTTTAAGAAGGGAAGAAAGAGAGAGAGAAAGAAAGATAGACAGAAAATGATAGATAG
ATAAACAGACAAATGATTGATAGATAAACAGACAGAAGACATATAGACAATGATAGACAGACAGATAGACAGTGAGATGTCTCAGCAGGTAAAG
GTAGTTGCTACCAAGCCTGAGGGCTGGAGTTGCCCCCCAAAAACACATGGTGGAAAAACTGGAATGGTATAAGTTGACCTCTGACCTTCACCTG
TGTGCCGAGCATGCACCCTCCCCCAGCGCCCACACACATGCACAGGAATAAAAACATAATTATTCTTCTTTTCATTTTTCAAGACAGCGTTTCT
CTGTGTAGCCCTGGCTGTCCTGGACTCACTCTGTAGACCAGGCTGGCCTCGGNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNGGAGTCTCAGTACCATCATCCCTGCTTATGAAATGACAGCAGCCCAGGCTGCCCCAGTTCCTCCTCTCTGCTCGACACTC
TCAGTACTCACTTCGGTCTGAAAATACCCACTCTGTGAAGAGGAAATTGAGACAAAGAAAAGCAACTTGCCCGCGGTTGCTAACTTGCCCCGTG
TGGATCCGAGCGTGCAGAGCCTGCATCCCTGTCGTTCACGGTGGTGTCCTCCTGACTAACTTCCTACAGGATTCTGATTTTACTTCCTAGGTTC
TCAGAAACCAACCAATACAATCAATGACATAGCCCTTTAACCCAAAATGAGAACTCCAATGAATGCTCATCTCAGGTGGCTTAGAGCCGGTTCC
CAGGGATGGTCGCTGGCTGTGGTTACAGTACACTGACCTCTCTGTGTGCTTATGGGGTTTTCACCCCACCAGTGGGCTCTCCTGTGGGAAGGTG
AATTCCGTTCAGAACCTTCTCTTAATGAATCAGGTAACCCAGCCTGGACCACAGATTGGGCCAGTGACTCCATTTGCATAGGATTTAAAAGAGG
ATTTCCTTCTGGGGCTCTTCTTTAGCCTGGCCAATTAAACACATGAGGTTCCTACTGGAACCAAATCAGAACAAAACTCCGTGCCCCAGGAGCT
AACCAGCCACTGTCTCCTGATGCAAATCAAAGCCTGTGCAAACAGAGGCCAACCCCCTGTCCCGCCTCTGGAAGTTATTCAAGCCCCCGACGGC
AGCTATGGTTACTCTAGGCATGATCACGTAAGTCCTTGAGGGCGATGCTTCTCAGTTCTGAGGAAAGCTCCCGTAAATGATTCTCCCCGCGGCC
TTGAACTTGTCGACCTCTGAGCAAATACAGAGCTCTTCCTTAACGAACGAACGGCATCGCTACGGCACTTGCCAATCCCAAGCACAAGCCTGTG
TCCAAGATGACTTTTAAAGAAGTGTATTTTGCCAGGAGTGGTGCCCTACGCCTTTAAACCCAGCTCTCAGGAGGCAAAGGCAGGTTGATTTGTG
TGTTTGAGGCCCTATTCTGCTTATGTCACCAGAGCTACATAGTGAGACCTTGTCTCAAAATAGAAAAACAGATGAGCTGGACGGTGGTGGCGCA
CGTCTTTAATCCCAGCACTTGGGAGGCAGAGGCAGGTGGATTTCTGAGTTCGAGGCCAGCCTGGTCTACAGAGTGAGTTCCAGGACAGCCAGGG
CTACACAGAGAAACCCTGTCTCAAAAAAGAAAAGAAAAAAAAAAGGAAAACAAATGATACATAGATAGATAGATAGATAGATAGATAGATAGAT
AGATAGATAGATAGAGAGAGAGAGAGAGAGATCGCTATAGGTAGGTAGGTAGATAGGTAGGTATAGACAGGTAGATAGATAAACAGATACTTAG
ATAGATGTTAAATATAGATAATAGATGATAATGGGTGGATAGATGGATAGATAGATAAGAAACGTATTTCTAAAAAGCTTCTACTTTTTCTTTT
TTTCTAGAACATCATTTTAATATTTTCAACTTAATATTCAACAGACAGAATAATTATTTTAAGATATATTTTGTTGTTATGTCTCCTTTTCATT
TCTGATGTTATTAATTTGAATACTGTCTCTGTGCCCTCTGGTTAGTCAGGCTAAGGGTCTATCTATCTTGTTTTTTCTCAAAGAACCAGCTCCT
GGTTTTGTTGATTCTTTATAGAGTTCTTTTTGTTTCTATTTGGCTGATTTCAGCCCTGAGTTTGACTATTTCCTGCCGTCTACTCCTCTTGCGT
GTATTTGCTTTGTTTTGTTCTAGAGCTTTCATGTGTGCTGTCAAGCTGCTAGTGTGTGCTCTCTCCAGTTTCTTTTTGGAAGCACTCAGAGCTA
TGAGTTTTCCTCTTAGCACTGCTTTCATTGTGTCCCATAAGCTTTGATATGATGTGCCTTCATTTTCATTAAATTCTAAAAACATAGACTTTAC
TTTCTTTTTTTATTTCTTCCTTGACCAAGTTATCATTGAGCAGAGCATTGTTCAGCTTCTGTGTGTATGTGTGCTTTCTGTTGTTTTTGTTGGT
ATTTAAGACCAGCCTTAGTCTGTGGTGATCTCATAGGGTATATGGGATTATTTCAGTCTTCTTGTATCTGTTGAGGCCTGTTTTGTGACCAATT
ATATGGTCAGTTTTGAAGAAGGTACTGTGAGGTGCTGAGAAGAAGGTATATTCTTTTGCTTTAGGATGAAATGTTCTATAAATACCTGTTAGAT
CCATTTCGTTCATAATTTCTGTTAGTTTCACTGTGTCTCGATTTAGTTTCTGTTTCCATGATCTGTCCATTGCTGAGAGTGGGGTGTTGAAGTC
TCCCGCTATTATTGTGTGGGGTGCAATGTGTGCTTTGAGCTTTAGTAAAGTCTCTTTTATGACTGTGGATGCCCTTGCATTTGGAGCATAGATG
TTCAGAACTGAGAATTCATCTTCTTAGATTTTTCCTTTGACCAGTATAAAGTGTCCTTCCTTATCTTTTTTGATAACTTTTGGTTGAAAGTTGA
TTTTATTCGATATTAGAATGGATACTCCAGCTTGTTTCCTGAAACAATTTGCTTGGAAAATTGTTTTCCAGACTTTTACTCTGAGGTCGTGTCA
GTCTTTGTCACTGAGCTGCATTTCCTGTGTGCAGCAAAATGCTGGGTCCTGTTTATGTATCCAGTCTGTTAGTCTATGTCTTTTTATTGGGGAA
TTGAGTCCATTGATGTTAATAGATATTAAGGAAAAGTGATTGTTACTTCCTGTTATTTTTGTTGTTAGAGGTGGAATTATGTTTGTGTGGCTAT
CTTCTTCTGGGTTTGTTGAAAGATTACTTTCTTGTTTTTTCTAGGATGTAGTTTCTCTTCTTGTGAAGTGTTTTCCATCTATTATCCTTTGTAG
CGCTGGATTTATGGAAAGATACTGTGTAAATTTGGTTTTGTCATGGAATATCTTGGTTTCTCTGTCTATGGCAATTTAGAGTTTTGCTGGGTAT
AATAGTCTGGGCTGGCATTTGTGTTCTCTTAGGGTCTGTATGACATCTGCCCAGGATCTTCTGGCTTTCATAGTCTCTGGTGAGAAGTCTGGTG
TAATTCTGATAGGTCTGTCTTTATATGTTACTTGACCTTTTTCCCTTACTGCTTTTAATATTATTCCTTTGTTTAGTGCATTTGGTGTTTTGAT
TATTATGTGATGGGACGAATTTCTTCTCTGGTCGAGTCTATTTGGAGTTCTGTAGGCTTCTTGTATGTTCATGGGCATCTCTTTCTTTAGGTTA
GGGAAATTTTCTTCTATAATTTTGTTAAAGATATTTACTGGCCCTTTAAGTTGGGAATCTTCACTCTCTTCTATAACTACTATCCTTAGGTTTG
GTCTTCTCATTGTATCCTGGATTTCCTGGATGTTTTGGGTTAGCAGCTTTTTTGCTTTTTGCGTTTTCTTTAACTGCAGTGCCAATGTTTTCTA
TGGTATTTTCTATACCTAAGATTCTCTCTTGTATTCTGTTGGTGATGCTTGCATCTATGACTCCTCATCTCTTCCCTAGGTTTTCTATCTCCGG
GTTTGTCTCCCTTTGTGATTTCTTTATTGTTTCTATTTCCATTTTAGATCTTGGATAGTTTTGTTCATTTCCTTCGCCTGTTTGATTGTGTTTT
CCTGTAATTCTTTAAGGGAGATATTTATCTCCTTCTTAAACTCCTCTATCATCATCATCAGGAGAAGTCATTTTAGAGCCCAATCCGGTGTGAT
GTGTATCCAGGACTTGCTATGGTGGAAGAATTCGGTTCTGATGATGCCAAGTAACCTCGGTTTCTGTTGCGTGTGTTCTTACACTTACCTCCTG
CCATCTGATTATCTCTAGCGCTACCTGCCCTGGCTATGTCTGACTGGAGCCTGTCCTTCCTGTGATCCTAGTTGTATCAGAGCTCCTCGGAGTT
CAGCTGTCTCTGGGATCCTGTGGTTCTGGGATTCTGAGATCCTGGGGTGTTCCTGGGTGCCTGGGTCCCTCCTGCTGGTCCCAGTTACTTCTGC
TCCTCCAACAGGTGTTGCTCTCCTCCTCCACCTGATCATATCCTCCTTTTCCATATCAAGCATTAAAAGTGTTCTTAGGTTTAAATTTTATCAT
CTAATGCTGGGCAATGGTGGCATACACCTTTAATCCCAGCACTCAGGAGGCAGAGGCAGGTGCATCTCTGTGAGTTTGAGGCCAGCCTGGTCTA
CAGAGCCAATTCCTGGACAGCCACGGGTGTATGAAGAAACCCTCCCTTGAAAAATAAAAATAAGTTGAGTAAATAAATAAATAAGTAGATATTG
TCATCCAATGGAAAACAAAAACTAAAACTTGGAAAGGCTGGAGAGACAGCTCAGTGGTTAAGAGCACTGCCTGTTCTTCCAGCCGATCCGCGTT
CAATTCCCAGCACCTTCATGTCAGCTCACACTTGTTTGTAACTCTAGTTCCAGGAAACACAGCACTTTTACACACAGACATCAACGCAAAACAC
CAAATAAATCTTTTTTTTTTTTAAGTGCCCTCAAGTTTTAAATTTTATCATCTAACAACAACAAAGAGGGGCTGCAGAGATTGCTCAGTGGTTA
GAGCACTGGCTGCTCTTGCTCAAGACCCAGGTTCAGTTCTCAGCACCCATATAGTGGCTCACATCCATCTACAATGTCAGTTTCAAGGGATCCG
ACACCCACTCCTGGCAGCCTCTGGCACTGTATGCATAAGGTGCACTTACATACATGCAGGCAAAACACCCATGCACATAAAATTAAAAACAAAA
CAAAACAAAACAAAAAAAAACTTTAAAAAAATCTCCAAGTCTGCATGTGGGGGAACTTCACCAGCCTCTCTTGAATCGTACACAGGCACGGCTG
CCGGTGAAGTGGATGGCACCCGAGAGCTTATTTGAAGGGATCTACACAATCAAGAGTGACGTCTGGTCCTACGGCATCCTTCTCTGGGAGATAT
TTTCACTGGGTATGTGTGGCACTCTGCAGGCCTCCTTGGGCTCTGGCTTTGTGTCCCCCACCCCTCTGTACCCTTCCTTTACCCTGCATGCCAA
GGGCCGTTAACACACCACTCAAGACCCTAGCTTCTAGTCTTGCCTCTGTTGCCAGACTTATTTGCTGACATCTGAGATCTTTCTTTCCCACACT
CTGTAATTGATTAAGAGGCTTGGGGCCCTCTGGATCCACTTGCAGCCCTCAGTACAGGGTGCGTCTGGCACTGGGGAATTCTCTTTGCTTGTTT
TCAGACAGAATGTATACCAAATAGACAAATAGGAAGAACAGTGTTTGTGAGGCCCCACTGAGCGCGTGTTTTTCTCAGGTGTGAACCCTTACCC
TGGCATTCCTGTCGACGCTAACTTCTATAAACTGATTCAGAGTCGATTTAAAATGGAGCAGCCATTCTATGCCACAGAAGGGATGTCAGTACAG
CCAGACCGCCGTTGGGTATCAACATTTACCGTGTGCTCTAAGTCCTCCAGATGCATTGCAAAAACAAAAAGCTTGATGGCTTTGTTGGTTTGTT
TGTTTCTTCTTTCTGTTTTGTTTTCTTTTTGAGCCAAGGTTCCCCTGTGTAGCCCTGGCTGTGGTGGAATTGACTCTGTAAACCAGGCTGGCCT
CAAACTCACAGATCCAGCTGTCTCTGCCTCCTGAGAGCTGTTATTAAAAATGGGCACCCCTGGGCTGGTGAGATGCCTCAGTGGTTAAGAGCGC
AGACTGCTCTTCTGAAACCCCCTAGTTCAAATCCCAGCAACTACATGGTGGCTCACAACCAAACGTAATGAAAACTCATGCCCTCTTCTGGAGT
ATCTGAGGACAGCTACAGTGTACTTACATATAATAAATAAATACATTTTTAAAAATGTGCACTGCCACCACACCTGGTTAAAACACACTTTCCT
ACGACATTGCTGAGATCTTATTTATTTATATTTATTTATTCATCTATCTATCTATCTATCTATCTATCTATCTATCTATCTATCTAATGTATGT
GAGTATACTGTCACTATCTTCAGAGATACCAGAAAAGGGCATTGGATCCCACTACAGATGGTTGTGAACCACCATGTGGTTGCTGGGAATTGAA
CTCAGGACCTCTGGAAGAGCAGTCTTAACCACTTAACCGCTGAGCCATCTCTCCAGCCCTTGTTTGCTGTTTAAAAGCAGAGACATCTTGGTAC
AGTGGCACCCACCTTTAATTCCCACAGCGAGGAAGCAGAGAGGCAGCCAGATCTCTGTGATTTGAGGCCAGCCTGGTCGACAGCCAAAGCTGTG
TAGTGAGATCCTGTCTTGAAAAAAACAATCGCCAATCAATCAATAAATTGGGGGGGGGGGGCAGGAAGGGCAAGGAGACACTTGCCTATAATCC
CAGTACTCCAGAGGTAAGGTATGAGGCTGTAGGCTAATCTGTGCTAATAGCAAGACAAGAAAGGAAGGAAGGANNNNNNNNNNNNNNNNNNNNN
NNNNNNNNNNNNNNNNNGAGGGAGGGAGGGAGGGAGGAAGGGAGAGAGAGAGAGAGAGAGAGAGAAAGAGAGAGAGAGAGAAAGGAAGGAAGGA
AGGAAGGAAGGAAGGAAGGAAGGAAGGAAGGAAGGAAGGAAGGAAGGAAAAGAAAGAAAGAAAGCTTCACGTAGCTTGCGCCCTCTTCGCCACC
CTCCAAGGCTTCATTCTTCTGCTTGTCGTACCTGAGGATTTGACTTCACCTCACTCTCTGGTGTGGTACCAATTCTAGTCTTCCCACAAAGCCA
GCAGCGCACAGTCCACAGCCCCCACCCCCCACCCCCATGCTGCCCTCGGTGTGAACTGTGGGTGGAGCCTTGCCAAGTTCATTCACACCCCCTC
AGCTCTGCTCTGTTTTGCTCTCTCACAGATACTTTGTAATGCAATCCTGCTGCGCTTTTGACTCAAGGAAGCGGCCATCCTTCCCCAACCTGAC
TTCATTTTTAGGATGTCAGCTGCCAGAGCCAGAAGAACCGCTACCTGGAGGCTTGGCTTAATCATAACGGTCCCCAGGGAGATGTGTGGGTGTC
ACACCGGGCACTAATGGCCTCAGATCCGGTTTAGCCCGACCATCTTTCTCACCTCACTTTGCCTAGATTACCCTTCCACAGTCCTGTGCAGAAG
ATGTACTATCTCCTGTGGGCTTGGGCGCCGGGCTTTCCTGTGATTTCATCCAAGTGATTAATAGGTTAAAAGCTCTTAAGGGTCCAGTGCAAAG
GGCTAGACAACTAAATAAACCTTAACTCCTCCAGGAGCCACTGGCCTTTTCTAAGTCAACATGGTGTGAGGGTGGGTATGGATGGGAAGGGCTT
TAGCCCAAAGCCCTGAGGACCCATTCAGTCTCTGCTGCTCATTCCCCACTGATCTGAACGATGGCAAGTTTCTTCTTTGGCCTCGGTGTTCCCA
TCTGTAAAATGGGTTGCAAAGGTAAATGATCCTCAAGCAGCAGGATCTTGGTATTACGTCATTAGCCCCTCCTCCTTAGCACTCTTAGCACAGG
AGAGGTCTGCTCCAGATAAGGGTGGAGCCTTGAACACTCAGACCCCAGGTTCAGATCCTGCCTCCGTCACTAGTGTCTCACATAGTGACTCACA
TAATGTCTCACATTAGTGACTTTAATGTCACTAATGTCTCACGTAGCATTTGCAACTTTAGACTTTAATGTGGACATCTTGTGGGATGATAGTT
CTGTAAAGGACAAATCACCTGCCATCTGCCCTTTTCCTACTTTTTATTAAAAATGAGTGGCGGGCTGGTGAGATGGCTCAGTGGGTAAGAACAC
CCGACTGCTCTTCCGAAGGTCCAGAGTTCAAATCCCAGCAACCACATGGTGGCTCACAACCATCCGTAACGAGATCTGACTCCCTCTTCTGGAG
TGTCTGAAGACAGCTACAGTGTACTTACATATAATTAATAAATAAATAAACAAATAAATAAATATAAATAAATAAATAAATAGATTTAGTCTTT
TAAAAAAAATCAGTGGCTGCTGTTTCAGAAAAAAACAAAACCCATGGAGGGGACTGCACAGATGGCTCAGTAGTTAGAGCACTGGCTGCTCTTG
CAGAGGATCAGGGTTCACTTCCCAGTAAACAGGGTAGCTCACAGTCACCTATACCTCCTATTCCAGGAGCTCCAGCGCCCTCTTCTGGCCATTG
CAGGCACTGCATTCAAATACTGACACAGGTGCACACTTGCACACATAAAGTAAATAAAACTTCAAAACAAGCCTGGAAACTACCCAAATTCATA
GTTTTATATTTTTAAGATTATTTTACTTTTCAATTACATGTAGTAGTGTTTACAGGGACCCTCAGAAATCAGAAGAGGGTCTCGAATCCCCTGG
GGCTGGAGTTACAAGCAATCGTGAGCCACCTCACATGGGCTCTTAGAACCAAACTCAGGTCCTCTTCAAGAGCAGCAGGTGTTCTAACCACTGA
GCCATCTCTCCAGCCCCTAAACTCATACTTCATGATCTGTATAAAAGCTAAGGTGTATTTAAGTACTGTGCTTTCTAGGCACACAGCGAGTCCT
GCTCAGCCGGTCATTCTTCTCCCTTTCCAGATGTATCAGAACATGGGTGGCAACGTCCCAGAACATCCATCCATCTACCAAAACAGGCGGCCCC
TCAGCAGAGAGGCGGGCTCAGAGCCGCCATCGCCACAGGCCCAGGTGAAGATTCACAGAGAAAGAAGTTAGCGAGGAGGNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNCCATGTATCTGAAATATAGTATA
TTCTTGTAAATACGTGAAACAAACCAAACCCGTTTTTTGCTAAGGGAAAGCTAAATATGATTTTTAAAAATCTATGTTTTAAAATACTATGTAA
CTTTTTCATCTATTTAGTGATATATTTTATGGATGGAAATAAACTTTCTACTGTAGAAATGTTGGTTCTTGTTCCTTTGCGTGAACTATAAGCT
CATGGCCTCATCTCTATGTGAGATTTCCTGTTTTTCTGTTTGTTTCTTTGTTTGTTTTTTCTTTCTCTTGAGCAGGTGCATTCTTGGGGGATGC
TTGGTGTTGTTTGGTTTTGAGACAGGATCTAGTTATATTGCTCAGGCTAGCCTCAAACCCGAGATCTTCCTGCCTCTGCCTCAAGTCTTGGGGT
TAACCTCATCACACCCGACGAAGGTCAGCTCTTCACCAAAGTGTTAAAGCATCCTGAGCCGACCAGGCTGAGGACCGCGGGCACCACATCTGAT
CGGTTCTCCCATTCCCTCCTCATTCACTTCTGATCTTTTGAAACTGAGCAAGCTCCAGCCCACAGGTTTATAAACTCGCCTTGCTTTGTCCCTT
TGTACGCTGGACACCACTGAGTTCTGTCCTTTATTTCTTTGTTTTGTCCTATGACAGGGTCTCATGTGTCCCAGGTTGGCCTCAGGTTGACTGT
GTACCACAGGATGACCTGGACCTTCTGGTCCTCGTGTCCCCACCTCCCGACTGCTGGGATCCCGGTGTGTGCCACCATGCCCGGCTTCGCCTGA
TTCTTTAGCTAGCCTCTTTGCCATCTAAGTCAGAGGTTCTCAACCTTCCTAATGCTGCTACCCTTTAATACAGTTCCTCATGCTGTGGTGACCC
CCAACCACGAAATTATTTCATTGCTACTTCATAACTGTAAGTTTGCTACTGTTATGAATCGCAATGTAAATAACCTGATGTGCAATGGCTAGAG
GGTCACGATCCACAGGTTGAGAATGCTGCCCTACTGTGTCGCTTAGCTGCTGCAAGTTCCGTCTCCCTAAGCATTGACTTGGTGTGTGGTATGG
TATGGTCTCTTGTAAGCACCAGGTCTCTCCATTGATCCAGCTTCCACCCCTCTGCCTCTTTGCTTCTTTCACCCCCTAGATACTACCTGCATAA
TGTTTTCTCTCTAGCCAAAACCGCCTGGATGGTCCTGGATGTGTCTGGGCGTTTCCCAGCCCCCTCTTCTGCCTCAGTCTCTGTTTCCATGCCC
ATCAAGAATAGCTGCCTCCGTGGCAACCTGTGAGATCTTCTTGTCTATATCATTAGGTTGTCTCTGCACAGACAGCCTACCTATGTGATGCTGT
AATTAGCTTTTGTGATAATGTCCTCTCTCTTCTCTCTTTATTTATTTATTTATTTACTTACTTACTTACTTAATGTATTTGAGTACAGTCATTG
TCTTCAGACACACCAGAACAGGGCGTCAGATCTCATTACAGATGGTTGTGAGTCACCATGTGGATGCTGGGAACTGAACTCAGGACCTCTGGAA
GAGCAGTCAGTGCTCTTAACCCCTAAGCCATCTCTCCAGCCCATGTCCTCTCCTTTAAGCTGGAGTTTGAGCCGTTTGAAGGCGGATGTGATTT
AAGTGAATCCAAACACTCAGCTCCACGGAGGTAATAGGTATAAACAGACATTTGGTTACTTTTTAGCCTACTCATGTAAAAGCATAAGATATGC
TTGATGGCAAACATTCAGCTAATATTGTTTTTTATTTAGGTGAGTGGTGGTTAGCCATGTAACAAGCGTTGGAACACCTCTCCAGCTGAGCTCC
GCTTCAGGTGTATCTCTCCTACCTTCCTGTCTGAATGTATTCCTACCACACACACATGAGCATGCGCAGACGCACACACATGAGCATGCGCAGA
CGCACATACATGAGCATACGCAGACGCACACACATGACCATGCGCGCGCGCACACACACACACACACACACACACACACACACACACACGAGTG
GCAAGGCGGGCCTGCAGCATGCAAATGGGAAAGAAGTCTTTGTATTAGTTACCTTACTCTTGCTATGATAAAACAGACCTTGGCTTGCTGCAGG
CTCTTGTTTACTGTTAATATTGCTGCTGAGAACATGGTATGGAAATGGAAATACCCCCCCCATCTCTCTCTCTCTCTGTCTCTCTGTCTGTCTG
TCTGTCCGTCTCTCTCTCTCTCTCTCACACACACACACTTTTTAAAATATTTTATTTTATTCTGAGACAAAGGTTTTTTTGTTTTGTTTTGTTT
TTGAGACAGAGTTTCTCTGTATAGCCCTGGCTGTCCTGGAACTCACTCTGTAGATCCCCTGCCTCTGCCTCCCGAGTGCTGAGATTAAACGTCT
GCCCCACCACCACAAGGGTTTTTTATCCACCCCAAGCTCGCCTTGAACTCGAAATCCTCCTCCTTGGCCTGCTGAGGTCCTGCACTGCTTCTTC
ACATTGGCCTTCACAGCTGTCTCTGTCGCTTACCCCTGAGAACCTAATGTGGAACCCGAGGCCAACTCTCAGCCTCCCAGGCACAGCCTAGTGG
TTTGCCGACGTTCCTTCTCTCTCTGTTAGCTACCAATGGTCTTTCTCGCTTTCTCTGTCTTCTCTGTCTTGATGGAATTGGAGGAGGCAGAAGA
GATACAGGAATTTGCAAAGCCCCTTGAGCTACTTGTCGTGAGATGTGTGTTGATAGAGCAAGGCAGAATAGTGATGTGAGCTACCCAAGCCTCT
TCCTTCTTTCAAAGTGATTTTGCCCAGGGCATGTGCACATTGTGTCTGTGACATCCCTCTGGATGGAGCCAAACACACAGTCACATCTCGAGTC
ATGTCCTTGTGTGCAGCGTTAAAACCCTCAAAGGACTTCATTTCCACGGAAATGGATATTCTCTTGTGGGTTTCGTAAAAGTGGTTTTATAGCC
TTAATACTACCAAATGCTTCTTTTAATAGCATAATATTGGGGCTGGAAAGACAGTTAATAGTCCATACTGCTATTGTGGAGGACCTGAGTTCAA
TTCCTAGCACTCACAACTGGAACATCACAGCCTCTGGCAACTCCAGTTCTGAGCGATCTGACGCCCTCTACTGGCCTCCATCGGCACTCCATGC
ACATAGTGCATACACACACTCAGGCACATACACATAGACATAAAATACATAAATCCTTCTTTTAAAAAGCGTAATATTGACTTGTTCTTCAAAG
ATGTATTCATGTTATGCAGTGATCATCCCTAATCTGAAATACTAAAACCCAGAATCTGAGCTTGATGAACATCCGCATAATACGTCACACACAC
ACACACACACACGGAAAAGAGAGAGAGAGAGAACTCGATCCCTGGCCTCCAATGAACACACGTTAGAATGAAGTGAGTTAAAATTATCACATCA
AGTTACCACCAGGCTGTGTACATAAGGCACACATGAAATCTTAACTGATCCTTTGAATTATATGTGCAAAATTATTCCAGTCATAGCAAGTTTA
GTCTGGTTCCTCTCACTCCAGCCAAGGGGTACTCTACCTGCCTTACCCCAAGCTGACATTGTCCATATTGCTGATGTCCTATGAAAACACACTC
CTTGACCACCATCTTCAAACTAAGGACATTTGAATATGCTTCTGGGCTGTGTCGGGTAGAGGTTTGGAGCATCTCCTTCATAGAAAGTGAAGTC
AGCAGAGGTGGAAACTTAAGGGCTCGTTGGAAAGTCAGTGGTGCGGGAAGGTGTTTGCTAAGGCAGACACGTGAAGGAATATTTAGCTGAAGCA
GACACAGGAGAAAAGGATGTTCTGCTAAGCCAAGCATGTGATGAAGGACTCTTTGCTAACAACACGCATGCGTTAGTCCATCTTACGTTGTGTA
GCTCTATTTGTCCGGAATCTATAGAGAGAAATGCACCAAAACAAAACAAAAACAAAAACAACTTCTGGTGGTGTATTGTGGCTTCTTGCTGCTC
CCAAGGACTCCGATTGGCAGAGTAATGTCAGCTGAGACAGTCGCACAGGATGAGGCAAGACCTGTGCAGGACACGTGATGATTGGATGGAGTAT
AAGTAGAACTCAACAGACAGTCATGGAGACTGAGCTTGGCTTGTTTATAGAGCTAGCTGTGCAATGCTTGTTGATCTCTTGTCTTCACCAGTCT
TCGATTCCCCGAGAGAGGCACAGCTAAGAACTTCTCCTGTGTCCCTCTGGGTCCTTCCTGCTGACTCTGCTAGGTAGGTTGTGCCACCGCTGCT
GATTCTCTTTGCTGTCCCCGCTCTTCCAAACTGGACTGCTGATGTTTCTGTGAAGTGTTTGTGCGTGGATCGAGCTGCTGACCTGTGAACTGAA
CTGCTAATTTCCAGACAGCACACACCGGAGTTGCTCTAAAGAACCTTTCTAAACAGGTCCACTTTCCCCGTATCCTCCCCCCCCCCCCACTTCC
TCTGGTGGGTGGTGGACTAAAACGGAGGTTAAAGCATTTAAAAGCCATCATTAAAAATAAGGATTGGCAGAGCCGCACTCAGGCACTGCAGCGC
AGCTCAGGTCTCCACCAGGCGCGCGAAGCTGAGCCAGAGCCAAGATCGCTGTCCTTTCTAAGGAATATGGATTTGTGCTTCTCACTCGTCCTGC
CACCTTTGATATGGTGCTCCACCTAGCCATCAACGTGGGCAAAGCCTGCAAGAAGGACAAGGTAGACTACCCTGTCACCTACAGCATGCATCCT
GAAAGTGGGCATATGTTCAGCTGCATTCAGCGCGCCCACCAGAACGTGTTGGAGGTACACCCTCCCTCCCTGTTTTTCCTAACGGTGGGAGGTG
TTAACTACCCCCGTATAGCTTCTGGCCTGGGCTTGGCCTTGATTTCTTGCACCAGTTCTTTACACATATCGACACCCTAGCAAGCGGTATCGAC
GAGCCACGGGGCTCTCTTGCCCTCTTTACTCTGATGGGCACCGCCATGTGCTCTCCTTCCCAGCATCCTCTCCTTCCCAGCATCTTGGCTGGAT
CAGACCGGGCTTAAGCTGCGGGTCCAGATCCTGCCACTGAGGAAAGGGGGGCCGGGGGGGGCCTTCCTATTCTCATTTGCCTCCTACGACTCAT
CCTGATTTCCAGTTCCACTGTTTAGTTTTTGTTGTTGTTTTAAATATAATAAAAACTTACCTGACATCAGCCTCATACCTAAAAATGAAGAAAT
AAAAATAAAAATAAGGATTGAAAAAGTTAAGGTTACAGTTAGGATCACAAACCATGCCCCTGTTCACTTTAGTGTGGGCCAAGCTGTGTCCCCT
GAGGGGAGGGTACCCCTCTTTTTCTTTCCCTTTCTTCCTTCCTTTCTGTCTCTTTTCTTCCTTCCTTCCTTTCTCTTTTAAAAATGTATTTATT
TTATTTGTATGGGTGCTCTATCAGTACCTGCGTGACAGAAGAGGGCATCAGATGCCATTACAGATAGTTGTGAACCACCATGTGGTTGCTGGGA
ATTGAACTCGGGGCCTCTCGAAGAGCAGTCTACTGGCTGGTTTTGTGTCAACTTGATACAGCTCGAGTTATCACAGAGAAAGGACCTTCAGTTG
AGGAAATGCCTCCATGAGATCCAGCTGTAAGGCATTTTCTCAATTAGTGATCAAGGTGGGGAGGGCCCTTTGTGGGTGGTGCCATCCCTGGGCT
GGTGGTCTTGGGTTCTATAAGAGAGCAGGCTGAAAGCCGGGCGTGGTGGCGCACGCCTTTAATCCCAGCACTTGGGANNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNATGGGCCCACTAGGGTGTGTGTGTGTGTGTGTGTGTGTGTGTGTGTGTGTGTGTAAAACAGTAATTATGGAAGATGTTAGTCATGAAC
TTGATAACCAGTTGTGGGAAGCATAGGGGGAGTTAGAAGGGAAAGAAAGGGGGGTAGATTTCAAATTTCTGAAAAAAAATTTTTTTAAAGGGAA
GACATGGCAACTAGTACCCTAGTTATGGCCCAGCAGGATCCCTCTCCATCCCCCACCCCACCCCTACCCCGTGAAGAACACCACCAGTAACAGC
CTTAGATAAACTGCTGCTGTGATAAAACACTTACCAAAAACAATCTAGGGAAGGCAAAGGTTTGTTTCAGTTTATAGTTTATAGGCTGTTGTTG
AAAGAATCCAACCCAAGAACTCAAGGTGAGAACCTGGAAGGCAGATTCAACCCTCTCTTCTTGGGCACAAACACACACACACACACACACACAC
ACACACACACACACGCTTGTACACATACAAATACACCTAATTAAAAAAAATTTTTTTTTAAACGAGACAAGATAGCACACTCCACCAGAGGCAC
CTTACCCTGTTCTGTTTACACTAATTAAGTCTATGTGGTGTTAAGCATTTAGAGTTCAAAGAAAGATTAAAACGCAGTGCCTGTGCTCTAGAGC
AACAGCAAGGACAGATGAGGTGCTTGTGAGTTACCGTGTCGGTGCTGGGGACAGAACCACGGTTGTCCCTGAGGACACTGTCCTGGTAATCCAG
GCAGGTCACAGGGCCACCTGTTTGGTCTGCATGTGGTACACAGATTACATGCAGGTAAAAGCATACGTGCACACAGACAAGATGGTAATGGCAC
CAAGTGTCTGTCTCATTACATACGGCCACCGCCGCCACCTCTTGTTATGGCAATGCCTCGGGTGGAATCATTCTTTCTGCAGAAGTCTCTGCTC
TCCTGATCATAGCCTTAAGCGGAGGTGGCGGATAGCTCAGTATTCGGGTGCTCCTCGAGCATGCGTGAGACCCTGGGTTCTGTCTCCAGCACAC
ATATTACCCCTACCACCAACAAAACAGTTAAACCTGTGTTTCTCATCGCAGGACCGTTCTGGATAAGCTCCTCCCAGAGATATTTAAACCAGGG
TCAGCTCCCCTTGCACAGGGCCAGCATGTGTGTTCAGCAAACACAACCCATGAGGAGGAATTACCTCACTCGCTCGATGACAGCGGACCACAGG
CAGGGCAGAACACTGCCTCCAGTCCAGGATCAGCCATGGACATGGTGAAGGTCAATTCCATGGACTTCGGAGAATTTGTGGATGTGTTTGGGAA
CATTGTTGAGAAATGCCCTCTGATTGCCGCTGCTGTCTGGTCCGAGCGTCCATTCTCTCGCTTGGAAGACTTAGAAAATCACTTTTTTGCCTTT
ATTGATGCTCTCCCGAGATCAGGTAAGACTTCAGGACCGTGTGCGCGAAGTCATTTTCAATATTGTATATAAAAATATTTTAGATTACTTTAGC
CTCCTTACTCAGTTATTTAGCCGGGATTATTAGAAGGATCAGTAGACATCCTTTCAAACGCTAGTCTCTTGAAGGTGGACACAAGAACATGGGG
GCGGGATCGGCAAGGTAGCATGGATGGCCGAGGACAGCGGAGGCAGACGCAGGACAATCTCTGAGGTCGAGACCAGCCTGATCTAGAGCGAGTT
CCAGGACGGCCAGGGCTATTGGAGGTCAAGAATGGGAACAGGGCAAAGGCCTTGCTGACTCAGAAGCTGAAGGCCCACTTTGGCAATGCCCTGG
ATGAAAGAGAGGATAGCGAGGACCTCCCAGGGACCCCAGAGAGGAACGGGAAGCCCTGAGCGATTTTCAAAAAAAAAGGGCCGGTTTGGACCAG
ACAAGGATGCATTCATTTAGATCCCAAGACTCTTTGCAGAGAGAAGTAAAATGGGAGCAAAGCAATGTTTTCTGGGGAGAGTGGGCCACAGGGT
GGGGAGAAGAGGGCGTAGAGGTTTATTCAGGATGGCACAGCGGCCGAACGTCCAAAGGTACATAGTCCCTGTTCATAGGCTACTATGGGTTCTG
AGAATAAACCCGACATGCCCTTGGGGAACAAAGTCAGAGGGTAGGAGTCACCTGCCCTTGAGACTGTTCTTAGTCTTCGGTCAGGAGGGTGGTC
TCACCACAGAGCAGGCACTCGCAGCTCCGTTCAGTATGGCTCTGACACTCTGTGGATTCTCAGACTCCTCTCTATGGCTGCCTATGAGAACTTA
CAGAGGAAGAGCACAGGTCCAGGTTATAAAATCAAGCTGAGGACTGGACAGATGGCTCAGCAGTTAGGAGCACTGGCTGCTCTTCCAGTGGACT
GGAGATCAATTTTAAGCACACATTCCCCACCCCCCAAAGATCCTCGCAAAGGCGTCTAACTCCAGGATCTCGTCTCCTGCTCTGGCCTCCGTAA
ACACCAAGGACACATGTGATGTGCAGACATACATGCAAACAAAACACTCAAATATGTGAAATTTAAGTGTGTGTCTGTGTGTGTGTGTGTGTGT
GTGTGTGTCTATCTGTCTGTCTATCTGTATGTATGTTTGCAAATCAACTAATCAACTCAGGGTCTTTCTTGCTTATCCTGGGCACTCTACCACT
AAGATGTACTCAATCTTTTTTCACAATGTTTCTAGAACAGAGAGAGAGAGAGAGGAGAGGAGAGGAGAGGAGAGGAGAGGAGAGGAGAGGAGAG
GAGAGGAGAGGAGAGGAGAGGAGAGGAGAGGAGAGGAGAGGAGAGGAGAGGAGAGGAGAGTCTCAATGTATTTGAAGGGTCATTTAAAAATCTT
TAATGGGAAGTGGTGGTGAAC
MOUSE SEQUENCE - mRNA
GCGGCCTGGCTACCGCGCGCTCCGGAGGCCATGCGGGCGTTGGCGCAGCGCAGCGACCGGCGGCTGCTGCTGCTTGTTCTTTTGTCAGTAATGA
TTCTTGAGACCGTTACAAACCAAGACCTGCCTGTGATCAAGTGTGTTTTAATCAGTCATGAGAACAATGGCTCATCAGCGGGAAAGCCATCATC
GTACCGAATGGTGCGAGGATCCCCAGAAGACCTCCAGTGTACCCCGAGGCGCCAGAGTGAAGGGACGGTATATGAAGCGGCCACCGTGCAGGTG
GCCGAGTCTGGGTCCATCACCCTGCAAGTGCAGCTCGCCACCCCAGGGGACCTTTCCTGCCTCTGGGTCTTTAAGCACAGCTCCCTGGGCTGCC
AGCCGCACTTTGATTTACAAAACAGAGGAATCGTTTCCATGGCCATCTTGAACGTGACACAGACCCAGGCAGGAGAATACCTACTCCATATTCA
GAGCGAACGCGCCAACTACACAGTACTGTTCACAGTGAATGTAAGAGATACACAGCTGTATGTGCTAAGGAGACCTTACTTTAGGAAGATGGAA
AACCAGGATGCACTGCTCTGCATCTCCGAGGGTGTTCCGGAGCCCACTGTGGAGTGCGTGCTCTGCAGCTCCCACAGGGAAAGCTGTAAAGAAG
AAGGCCCTGCTGTTGTCAGAAAGGAGGAAAAGGTACTTCATGAGTTGTTCGGAACAGACATCAGATGCTGTGCTAGAAATGCACTGCACCGCGA
ATGCACCAAGCTGTTCACCATAGATCTAAACCAGGCTCCTCAGAGCACACTGCCCCAGTTATTCCTGAAAGTGGGGGAACCCTTGTGGATCAGG
TGTAAGGCCATCCATGTGAACCATGGATTCGGGCTCACCTGGGAGCTGGAAGACAAAGCCCTGGAGGAGGGCAGCTACTTTGAGATGAGTACCT
ACTCCACAAACAGGACCATGATTCCGATTCTCTTGGCCTTTGTGTCTTCCGTGGGAAGGAACGACACCGGATATTACACCTGCTCTTCCTCAAA
GCACCCCAGCCAGTCAGCGTTGGTGACCATCCTAGAAAAAGGGTTTATAAACGCTACCAGCTCGCAAGAAGAGTATGAAATTGACCCGTACGAA
AAGTTCTGCTTCTCAGTCAGGTTTAAAGCGTACCCACGAATCCGATGCACGTGGATCTTCTCTCAAGCCTCATTTCCTTGTGAACAGAGAGGCC
CAAAATGTTCACGCTGAATATAAGAAAGAAACCTCAAGTGCTAGCAAATGCCTCAGCCAGCCAGGCGTCCTGTTCCTCTGATGGCTACCCGCTA
CCCTCTTGGACCTGGAAGAAGTGTTCGGACAAATCTCCCAATTGCACGGAGGAAATCCCAGAAGGAGTTTGGAATAAAAAGGCTAACAGAAAAG
TGTTTGGCCAGTGGGTGTCGAGCAGTACTCTAAATATGAGTGAGGCCGGGAAAGGGCTTCTGGTCAAATGCTGTGCGTACAATTCTATGGGCAC
GTCTTGCGAAACCATCTTTTTAAACTCACCAGGCCCCTTCCCTTTCATCCAAGACAACATCTCCTTCTATGCGACCATTGGGCTCTCGTCTCCC
TTCATTGTTGTTCTCATTGTGTTGATCTGCCACAAATACAAAAAGCAATTTAGGTACGAGAGTCAGCTGCAGATGATCCAGGTGACTGGCCCCC
TGGATAACGAGTACTTCTACGTTGACTTCAGGGACTATGAATATGACCTTAAGTGGGAGTTCCCGAGAGAGAACTTAGAGTTTGGGAAGGTCCT
GGGGTCTGGCGCTTTCGGGAGGGTGATGAACGCCACGGCCTATGGCATTAGTAAAACGGGAGTCTCAATTCAGGTGGCGGTGAAGATGCTAAAA
GAGAAAGCTGACAGCTGTGAAAAAGAAGCTCTCATGTCGGAGCTCAAAATGATGACCCACCTGGGACACCATGACAACATCGTGAATCTGCTGG
GGGCATGCACACTGTCAGGGCCAGTGTACTTGATTTTTGAATATTGTTGCTATGGTGACCTCCTCAACTACCTAAGAAGTAAAAGAGAGAAGTT
TCACAGGACATGGACAGAGATTTTTAAGGAACATAATTTCAGTTCTTACCCTACTTTCCAGGCACATTCAAATTCCAGCATGCCTGGTTCACGA
GAAGTTCAGTTACACCCGCCCTTGGATCAGCTCTCAGGGTTCAATGGGAATTCAATTCATTCTGAAGATGAGATTGAATATGAAAACCAGAAGA
GGCTGGCAGAAGAAGAGGAGGAAGATTTGAACGTGCTGACGTTTGAAGACCTCCTTTGCTTTGCGTACCAAGTGGCCAAAGGCATGGAATTCCT
GGAGTTCAAGTCGTGTGTCCACAGAGACCTGGCAGCCAGGAATGTGTTGGTCACCCACGGGAAGGTGGTGAAGATCTGTGACTTTGGACTGGCC
CGAGACATCCTGAGCGACTCCAGCTACGTCGTCAGGGGCAACGCACGGCTGCCGGTGAAGTCGATGGCACCCGAGAGCTTATTTGAACGGATCT
ACACAATCAAGAGTGACGTCTGGTCCTACGGCATCCTTCTCTGGGAGATATTTTCACTGGGTGTGAACCCTTACCCTGGCATTCCTGTCGACGC
TAACTTCTATAAACTGATTCAGAGTGGATTTAAAATGGAGCAGCCATTCTATGCCACAGAAGGGATATACTTTGTAATGCAATCCTGCTGGGCT
TTTGACTCAAGGAAGCGGCCATCCTTCCCCAACCTGACTTCATTTTTAGGATGTCAGCTGGCAGAGGCAGAAGAAGCATGTATCAGAACATCCA
TCCATCTACCAAAACAGGCGGCCCCTCAGCAGAGAGGCGGGCTCAGAGCCCAGTCGCCACAGCGCCAGGTGAAGATTCACAGAGAAAGAAGTTA
GCGAGGAGGCCTTGGACCCCGCCACCCTAGCAGGCTGTAGACCGCAGAGCCAAGATTAGCCTCGCCTCTGAGGAAGCGCCCTACAGCGCGTTGC
TTCGCTGGACTTTTCTCTAGATGCTGTCTGCCATTACTCCAAAGTGACTTCTATAAAATCAAACCTCTCCTCGCACAGGCGGGAGAGCCAATAA
TGAGACTTGTTGGTGAGCCCCCCTACCCTGGGGGCCTTTCCACGAGCTTGAGGGGAAAGCCATGTATCTGAAATATAGTATATTCTTGTAAATA
CGTGAAACAAACCAAACCCGTTTTTTGCTAAGGGAAAGCTAAATATGATTTTTAAAAATCTATCTTTTAAAATACTATGTAACTTTTTCATCTA
TTTAGTGATATATTTTATGGATGGAAATAAACTTTCTACTGT
MOUSE SEQUENCE - CODING
ATGCGGGCGTTGGCGCAGCGCAGCGACCGGCGGCTGCTGCTGCTTGTTGTTTTGTCAGTAATGATTCTTGAGACCGTTACAAACCAAGACCTGC
CTGTGATCAAGTGTGTTTTAATCAGTCATGAGAACAATGGCTCATCAGCGGGAAAGCCATCATCGTACCGAATGGTGCGAGGATCCCCAGAAGA
CCTCCAGTGTACCCCGAGGCCCCAGAGTGAAGGGACGGTATATGAAGCGGCCACCGTGGAGGTGGCCGAGTCTGGGTCCATCACCCTGCAAGTG
CAGCTCGCCACCCCAGGGGACCTTTCCTGCCTCTGGGTCTTTAAGCACAGCTCCCTGGGCTGCCAGCCGCACTTTGATTTACAAAACAGAGGAA
TCGTTTCCATGGCCATCTTGAACGTGACAGAGACCCAGGCAGGAGAATACCTACTCCATATTCAGAGCGAACGCGCCAACTACACAGTACTGTT
CACAGTGAATGTAAGAGATACACAGCTGTATGTGCTAAGGAGACCTTACTTTAGGAAGATGGAAAACCAGGATGCACTGCTCTGCATCTCCGAG
GGTGTTCCGGAGCCCACTGTGGAGTGGGTGCTCTGCAGCTCCCACAGGGAAAGCTGTAAAGAAGAAGGCCCTGCTGTTGTCAGAAAGGAGGAAA
AGGTACTTCATGAGTTGTTCGGAACAGACATCAGATGCTGTGCTAGAAATGCACTGGGCCGCGAATGCACCAAGCTGTTCACCATAGATCTAAA
CCAGGCTCCTCAGAGCACACTGCCCCAGTTATTCCTGAAAGTGGGGGAACCCTTGTGGATCAGGTGTAAGGCCATCCATGTGAACCATGGATTC
GGGCTCACCTGGGAGCTGGAAGACAAAGCCCTGGAGGAGGGCAGCTACTTTGAGATGAGTACCTACTCCACAAACAGGACCATGATTCGGATTC
TCTTGGCCTTTGTGTCTTCCGTGGGAAGGAACGACACCGGATATTACACCTGCTCTTCCTCAAAGCACCCCAGCCAGTCAGCGTTGGTGACCAT
CCTAGAAAAAGGGTTTATAAACGCTACCAGCTCGCAAGAAGAGTATGAAATTGACCCGTACGAAAAGTTCTGCTTCTCAGTCAGGTTTAAAGCG
TACCCACGAATCCGATGCACGTGGATCTTCTCTCAAGCCTCATTTCCTTGTGAACAGAGAGGCCTGGAGGATGGGTACAGCATATCTAAATTTT
GCGATCATAAGAACAAGCCAGGAGAGTACATATTCTATGCAGAAAATGATGACGCCCAGTTCACCAAAATGTTCACGCTGAATATAAGAAAGAA
ACCTCAAGTGCTAGCAAATGCCTCAGCCAGCCAGGCGTCCTGTTCCTCTGATGGCTACCCGCTACCCTCTTGGACCTGGAAGAAGTGTTCGGAC
AAATCTCCCAATTGCACGGAGGAAATCCCAGAAGGAGTTTGGAATAAAAAGGCTAACAGAAAAGTGTTTGGCCAGTGGGTGTCGAGCAGTACTC
TAAATATGAGTGAGGCCGGGAAAGGGCTTCTGGTCAAATGCTGTGCGTACAATTCTATGGGCACGTCTTGCGAAACCATCTTTTTAAACTCACC
AGGCCCCTTCCCTTTCATCCAAGACAACATCTCCTTCTATGCGACCATTGGGCTCTGTCTCCCCTTCATTGTTGTTCTCATTGTGTTGATCTGC
CACAAATACAAAAAGCAATTTAGGTACGAGAGTCAGCTGCAGATGATCCAGGTGACTGGCCCCCTGGATAACGAGTACTTCTACGTTGACTTCA
GGGACTATGAATATGACCTTAAGTGGGAGTTCCCGAGAGAGAACTTAGAGTTTGGGAAGGTCCTGGGGTCTGGCGCTTTCGGGAGGGTGATGAA
CGCCACGGCCTATGGCATTAGTAAAACGGGAGTCTCAATTCAGGTGGCGGTGAAGATGCTAAAAGAGAAAGCTGACAGCTGTGAAAAAGAAGCT
CTCATGTCGGAGCTCAAAATGATGACCCACCTGGGACACCATGACAACATCGTGAATCTGCTGGGGGCATGCACACTGTCAGGGCCAGTGTACT
TGATTTTTGAATATTGTTGCTATGGTGACCTCCTCAACTACCTAAGAAGTAAAAGAGAGAAGTTTCACAGGACATGGACAGAGATTTTTAAGGA
ACATAATTTCAGTTCTTACCCTACTTTCCAGGCACATTCAAATTCCAGCATGCCTGGTTCACGAGAAGTTCAGTTACACCCGCCCTTGGATCAG
CTCTCAGGGTTCAATGGGAATTCAATTCATTCTGAAGATGAGATTGAATATGAAAACCAGAAGAGGCTGGCAGAAGAAGAGGAGGAAGATTTGA
ACGTGCTGACGTTTGAAGACCTCCTTTGCTTTGCGTACCAAGTGGCCAAAGGCATGGAATTCCTGGAGTTCAAGTCGTGTGTCCACAGAGACCT
GGCAGCCAGGAATGTGTTGGTCACCCACGGGAAGGTGGTGAAGATCTGTGACTTTGGACTGGCCCGAGACATCCTGAGCGACTCCAGCTACGTC
GTCAGGGGCAACGCACGGCTGCCGGTGAAGTGGATGGCACCCGAGAGCTTATTTGAAGGGATCTACACAATCAAGAGTGACGTCTGGTCCTACG
GCATCCTTCTCTGGGAGATATTTTCACTGGGTGTGAACCCTTACCCTGGCATTCCTGTCGACGCTAACTTCTATAAACTGATTCAGAGTGGATT
TAAAATGGAGCAGCCATTCTATGCCACAGAAGGGATATACTTTGTAATGCAATCCTGCTGGGCTTTTGACTCAAGGAAGCGGCCATCCTTCCCC
AACCTGACTTCATTTTTAGGATGTCAGCTGGCAGAGGCAGAAGAAGCATGTATCAGAACATCCATCCATCTACCAAAACAGGCGGCCCCTCAGC
AGAGAGGCGGGCTCAGAGCCCAGTCGCCACAGCGCCAGGTGAAGATTCACAGAGAAAGAAGTTAG
HUMAN SEQUENCE - GENOMIC
CACTGAGACGCACATGTTATCTTTCAAACAAGTCAATGGACTAGAATAAATCATCCTTTCTCAGCATGTGGTCTGTTGAACAACTGTGTAAGAA
TCATCTGAGCAAATGTTTAAAATGTGGATTCCTGGCTCCCATTCCAGACATGCTGACTCAAAACTGGGGCAAAACGCTCAGACTCTGCTTTTTT
AATGAGAACCTTTGGTGATTCCTATCACAGGTCTAGATCTGTGCTGTCCGGTACGGTAGCCACCAGCCACATGTGGCTATTGAGAGCTTGAAGT
GTGTCTGGTCCAAATTGAGATGTACTCTAATTGTAAAATACACACTAGATTTTGAAGACTTAGTATGAAAAAAAGAATGTAAAATATATTATTA
ATAATTTTTATATTGGCCGGGCTCGGTGGGTCACACCTGTCATCCCAGCACTTTATGAAGCCAAGGCGGGTGGATCGCTTGATCCTAGGAGTTT
GAGACCAGCCTGGATAACATGGCAAAATCCCATCTCTACAGAAAATACAAAAATGAGTTGAGTGTGGTGGTGTACACCTTTAGTCCCAGCTACT
TGGGAGGCTGAGGTTGGAGGATTGATTGAGCCCAGGTGGTCGAGGCTGCACTAGCCATGATCACGTCACCGCACTCCAGCCTGGATCCACAGCA
AGAACCTGCCTCAAAAAGAAAAAATAATAATAAAATTAAAACAATTTTTACATTGATTATATATTAAAATGATAGTATTTTGGATATATCGCAT
TCAGCGAAATATGTTATTAAACTTAATTTCACCTGCTTCCTTTTACTTTATACAGCCAACAGAATTTGTAAATGATGTACAAGGCTTGAAGTTG
TGACTCACATTCTGTTTCATTGGCTAGCCCTGTTCTAGATGAAGAGTAGGAGTTCTTTTCACCTTGATTCCGCTAAGTGTTTGTTTCATGATTA
TTGTTACTCTTTATACCTAAAAGGGATTTGTACTAAAAGTCCCTTGTTGGGACTTTTAGAATCTCAGTGCAGGGTTTGCATGGAGTTGTCTACT
GTCTCTTACTGAAACCCCCCAATGTTCAGAGCATTCATTTTTAAAGTAATTAAGAATGCAGACTGGTCTGGGAGCTGACTGAAACACACTCCCC
CAAACTCAGGGTTTGTCAACAGTGGCGCTGCTGGCATGTTGAGCTGGATAATTCTTTGTTGTGAAGGACTGTCCCGTGCACTGTAGGATACTTT
TCAGCATCCCGGGTCTCCTCCCACTGGATGCCAGGAGCACATTCCCTTTTCCCAGTGTGACCAAAAATATCTCCAGACACTGCCAAATTGAGAA
AAATAACCCCTGGCATTTTTTTTTTTTTTTTTTTTTTGAGAACCATTGCCTTAGAGGCATCAACCCACAGTCATCGTAAGCTGTTATTCAGAAA
TATCAAGCTTTTTTTTTTTTTTTGAGACAGAGTCTTATTCTGTTGCCCAGACTGAAGTGCAGTGGCACGATCTTGGCTCACTGCAAGCTCCTGG
GTTCAAGCAATTCTCCTGCCTCAGCCTCCTAAGTAGCTGGGATTATAGGTGCATGCCACCATGCCCAGCTAATTTTTTGTATTTTTAATAGAGA
CGGGGTTTCTTCATGTTGGCCAGGCTGTTCTCGAACTCCTGACCTCAAGTGATCCAACCGCCTCACCCTCCCAAACTGCTAGAATTACAGGCGT
GAGCCACCGCGCCTGGCCCATCAAGCTTTTAAATGTAGAAGGTTGGAGAGTCAGCCCCTCTTTACCCTAGTTGACTTGATTACCACTTGGCCTG
TGTTCAGAAGGAAATCAGCATATCACTGTCCCCTCTCTCCTTTGTATCCTATCCCCATGTTAGATAAGCTCTTAGCATTATGGGAATCTCTTCT
GAACCTGATTCAAAGAGAGAAGGGGTCGATTCAATTTAGTTCAACAAATATTACTTTAATATCCTCTTGATGGGTGTGATGCTGTGATGGGCTG
CAAGAGTGAATAAGCTAATGTTGCTTTCCCTCTCCCGCAAGAATGTACACAGATTGCTACTGTGCTGGTGATATCTGGTATAACCTTTTGAGAT
TTTACAGAATCGCCAGTTGGCGACCATGTCACTGACATATGGGTCATGGGCCAATGTACTGGGCTGCTTCATGTAGTTTCATTCCATGGCTACA
ATCCATACCCATGACCCCAGATTGTTATCCTATAGAGGTAGCCAAATGAATGAATTAGGTGAGCTCCTGACTCCTATAGCGTTTTGTACCTCAC
TCATTCCTACTTATCTGGCAACAGATAAGTATGTATGTCACATCTCACCAGGGAGTTTATAAACTCTAAGAATACGTATGCTGCCATATTTCTC
TATCTTTGTACAATGCCTTGTTCAGTAAGTGTGTTTAATAAATGAATGAATTCATCATCATATCTGGAATGCAACTCACAGTGCAGTCTGGGCC
AAAGATGAGTCGACAGCATCCTCTTCACTGCAAGGACCACGCACATGGTTCTTAGCACAACACCCAGTACTCAGTGTTTATTGTTATTTGAAAG
TTCTTGAAGAGCTACAGTGTTTGTGCTAATGATCTGCATTGTTGGTACTGATAATGTACCAGGTGTTATGGGCCACTCACGACACACAGCAAAG
GAAAGATGGGCCCTCCTTCTTCATGAGAAAACATGCTCAAAGTACTGAACAAGAAACGAACAACACAAAATTTGAACACGCCTCTGAGTGCTCT
AAATGTCTTTTTCTCTCCACATGAGGCTCTATCTTGAGATGGGGATGGAGGACTAGATTATAGTCAGGGGTAATTTCCACTTTTCTTTGTTTTT
GCCTTTTTTTTTTTTTTGAGACAGAGTCTCACTCTGTCGCCCAGGCTGGAGTGCAGTGGCATGATCTTGGCTCACTGCAACCTCCGTCTCCTGG
GTTCAAGTGATTCTCCTGCCTCAGCCTCCCTAGTAGCTGAGATTACAAGCATGTGCCACCCTGCCCAGCTATTTTTTTGTATTTTTAGTAGAGA
CGGGGTTTTGCCATGTTGGCCAGGCTGGTCTCGAACTCCTGACCTCAAATGATACGCCCACCTCGGCCTGCCAAAGTGCTGGGATTACAGGTAT
GAACCACCGCGCCTGGCCCTTCTTTTTGCTTTTTTTTTTGAGACGGAGTCTCGCTCTGTCACCCAGGCTAGAGTGCAGTGGCGCAATCTTGGCT
TACTGCAACCTCCTCCTCCTGGCGGAGGTGATTAAGTGATTCTCCTCTCGGTGGAGGTGAGTAAGTGATTCTCCTGCCTCAGCCTCTCGAGTAG
CTGGGATTACAGGCATGCACCACCACACTCGGCTAATTTTTGTATTTTTAGTAGAGACGGGGTTTCACCATGTTGGCCAGGCTGGTCTCAAACT
CCTGACCTCGTGATCTGCCCGCCTTGCCCTCCCAAACTGCTGGGATTACAGCCGTGAACCACCTTTCCTGGCTTCTTTTTGCTTTTTAGAGTCA
GGGTCTTGCTATATTGCCCAGGTTGGATTCTAACTCCTGGGCTCAAGTGATCCTCCCGCCTCAGCCTCCTGAGTAGCTGCAACTTGGCGTGTGC
CACTGCACCCGTCTTTCCTTTTGACTTTTTATTATAGAAAGTTTCAAATTTACTAATAAGTTGAAATAATAGTGCATGAGCATTCTTTACCTAC
CACCTAGATTCAACAATTTAATTGAACCGAATTTGGTTTGTGAAGTACAATATTTTAGTCATGTATCAAGATTTTATAGGCAAAGTGATACAAT
TTTTTAAAAATCTGTTACTGAAAGGCCCCTGTAATAACACTACCCCTGAAAAAACTTTGGTGTGGTCATGGTATGTGCACCAGAAATCTCTCGG
CTTCCCCACTTTCATGGCTTCATTTCTCCAATTTACTTCCTCAAATATTACGCCAAAGTTTACTAGGGCTTGCCTTTATGTTTTATTAAATTAT
AACTTTCTAGCCGTCCTGGTGTCCTGGTCCTGATAAAGGTTTCCAGGATATTTCACCCATTAGTTTGTATAATTTCCTATCTCTACAGACTTAA
TTACAAATTCCAAATCTACCTTAGCTTTTTCCACTTTTATGGGAAAAAAAAAAAAAATAGGACTGTTGTAATTACTTAGTAACCTCTTCTGAAA
CCTTCTCCTTAGCCTTGCCTAAGCCTGATGTTTTCCTTTTTCCTGAAATATTTAATTTTCCAACTCTGTGAATCTAAGTTTTGCCCACAGTAGG
CACTAACCGAGTGCTTACGGGGTAAACAGCTGTGTGGTGGTCGTTTGGGTTCTGGGTGGTGAGCCAGGGTCCTGCTCAGAAGAACCACAAACTG
AAAAGCAGGCTCTCACATACAATGAGTGACACACAATTGCAGAAGATTATGGAGCCTGGGAGAGGAGTTGTTTTTTTTTTTTTCCTATTTTCTG
AAGGAGTTGAGCCTTGAGCACAGACAGAAAGGAGGGAAAATGAGGGAAAAGTTTGAGTCTAGAGAAATACCAGCAGCTCCTACCTAAATGCCGT
TCCCATTCACTTCTGCAGTTTGAGACAACATTCCTGGCTGCAGGTCTTCCCAAGACCCCACTCAGTTGTAAGTTATTAACCACATCTTAAGAAG
CCATTCTGTGAGAATAAAAGATCCTTCTTTGAGCTCTTACCATGTGCAAAATCCTATAGGTGTATTACCTCATTTAACTCTCATAATCCCATTT
TATGAAATGTGGAAATTGAGGCTAAATTACTTGCCCAATCACAGAAGGCAAAGCCAACACCCTTTATTCCTTCTTTTTTTCTGAAATTGCCTTA
TAAAAACACATAATTCCACCTCCAGAGATAACATTGCAGACTTTTTCTATGTAAGCACCATGATAGAAACATACAATATGGATCTTTGTATAAA
CATGAAATAAGGTACCATTTGTAATCTGATTTTTCACTTAAAATATATCATGAGTATCTTTCTATATTAATAAGTATGTTGTTATATCTACCTT
ATTGCTGTAATGGCTGCATAGTATTCCACTTTTCAGAAATTTTTTATTTTAACTAACTCCTGTTGATGGGCATCAACTTACTTTTTTTTCTTTC
TCTTTCTTTGACATTTTTATTTATTATTATTATTATTATTATTATTATTATTATTATTATTATTATTTCGGATGGAGGCTTGCCCTCTCACCCA
TGCTGGAGTGCAATGGTGCGATCTCGGCTCACTGCATCCTCCACCTCAAGCGATTCTCCTGCCTCAGCCTCCCGAATAGCTGGGATTACAGGCG
TGTGCCACCGTGCCCAGCTAATTTTTTGTATCTTTAGTAGAGACGCAGTTTCACCATGTTGGCCACGCTGATCTCGAACTCCTTACCTCAAGTG
ATCCTCCTCCTTTGGCCTCCCAAAGTGTTGGGATTGCAGGCGTGAGCCATCGTTCCCAGCCAAGAAACCCCTTTTCTTAGTTTTAATGAGAACG
CATCTTCTTAGACACCGAGGTATTGTTTTACTGTAAACTCAAATGTCCTGACCTCCGTCTCCTGCCGGCTGGCCATTTGTAGAAAACCCCTAGG
CGAAAGAGGAAGCTTTGCATCTCCCAATCAGCCTAATTAGTTAGTCCCCTTGGAATTCCCATGGGGTCAGGACACGGTTGTCATTGCAGTTGTC
ATTGTTTCCAGGGTTAACTGTCATAAGCATGAGGAAAGGCAGTTTAAAGCCATTGGATATTCAGTTTTGTGTTCCTGCTATTTATGTGACCAAT
TTCATACTTGGAGTTTGTTCAGAAGAGTGAAAAATGTTTCAAGTGCTATCTCTTCTAAACCATGGCAAACCACACTCTATTAAGACAGCACAAA
AGACTGAGGAAGAAAAATGTTTACGAAACATTTTGCTTTGGTCTGCCTCATGATAAGTAATTGTACTGGTCATTTCTTTTTCCACTGAATTGAC
TGGTGTCTCTGACATGCTCTCTGGTGAGCCACACTGGCTTCATTACCTCCTCTCTTCTCCTTTTTCAGGGTCTTTGGCCATTGTCCAGAGAGAA
TTACACTTTCTCTCTTAGAAGCTCACGTCTTTCTTCTCTTGGGCCACTTCCACGCCAGCCTCTTCCACACTGCCTTCCTGAGGTCGGGCACTGG
CATGGAGCTCTGTTTCCTCTTCTCCTCCCTAGGGTTGTAATCTGGCTTTCCCCCAATCTTTCTATTGTTTCCACTTCACCAGTGATTTCCTCCT
TGTTGTCCAGAAATAGGTCAGGATAAGAAATTCCCCTTTTTGACTTCTTCAGCCTTCTTGTTAACAAAGCAAAAGAAGGTTAAACACCTCATTC
AATGCTCAGCATTTTTTTTTTTTTTTTTGAGTCCGGGTCTTGCTCTGCTGCCTAGGCTGGAGTATACTGGCTCCAGCAATTCCCCTACCTCGGC
CTCCCAAGTAGCTGGGACCACAGGTGTGTGCCATCGCACCCAGCTAATTAAAAAAAAAAAATTCTGTAGAGACAAGGTCTTGCCATGTTGCCCA
CGCCGATCTCAAACTCCTGGGCTCAAGTATTCCACCCTCCTCGCCCTCTCAGAGTGCTGGGATTACAGATGTGAACCCCTGCACGCACCCAAAG
GTCAACTTTTAACTGCCTGAAACTTTTGATAGGTCAGAGAGATGAATTCTCTTGTTCACAATACACAGTCAGAATTACCTTGGCTTACGAGTTC
AGGGCACAGAACACTGGTGTCACACAAACGTGGGCTCAAAAGCTGCAATTACGCAGTGGCGTTTTCTGGATTTTTTTTGTAGAGATGGGATCTC
AGTAGATTGCCAAGGCTGGTCTCAAACTTCTGGCCTCAAGCAGTCCACCCATCTCACCCTCCCAAAGTGTTGGGATTACACGTGTGTTTTCAGT
CTTGGCGACCAACTGGGATTCTCTTCTCCCCTTTATCTTCTGTATCCAATCTAGCCCCTGCCATTTCTTTTTTTCTGGTGCCTCTAGGGTTTGC
TCTTTTCTCTCCTTTCCCATTCTCCTCCATTAAACAACGCTCCCCTTGTCATGCTCCGAAACCTCAAATCTGAACTTTTACTTCTCCTATCACA
GAAACTTCCCACACTCCAACTGCTTATTACCCCTCTACCTTCACCACCAAATTGTAAATTCTGTGAGAACAGAAACCCAATTTTTTGAATTTGC
TGTTGTGTCCTCAATTTCCGTCACAAACAGGTTCTAACATTGACAGTTGAATGAAGGAATGAATGAACGAGTTCTTGGATTGCTACAACTGCCT
TTAAACTTCCAGTAAACTTTTGCTGCCATCAGCAGGTGCATATGGGGCACTCTTCATAGGCCAAGCCACCCTGACTCATACCTGTTATCCCAGC
ACTTTGGGAGGCCGAGGTGGCGAGGATGGCTTGAGGGCAGGAGTTTGAGACCAGCCTGGGCAACATAGTGACACCCCATCTGTACAGAAAAATT
ACATAAAATAAAATTAAGGCTAGGTGCAGTGGCTCATGCCTGTAATCCCAGCACTTTTGGAGGCCAAGGAGAGAGGATCACATGAGGTCAGGAA
TTCAAGACGAGTCTGGCCAACATGGTGAACCCTGTCTCTATGAAAAATATAAAAATTAGCTGGCTGTGGTGGCGTGTGCCTGTAATCCCAGCTA
CTTGGGAGGCTGAGGCAGGAGAATTGCTTGAACCCAGGAGGCAGAGGTTGCAGTGAGCCAAGATCACGCCACTGAACTCCAGCCTGGGCGACAG
AGTGAGACTCCATCTCAAAAAAAAAAAAAAAAGTTTTATTGCAATGCTCAGGGCAGTAAGCATTGTTGTAGAAAATAAGTAATTTGTATTTTTA
AAATGCGTAATTCATCCCGGCACTTTGGGAGGCTGAGGTGGGTGGATCACTAGGTCAAGAGATCAAGATCATCCTGGCCAACATGGTGAAACCC
CGTCTCTACTAAAAATACAAAAAATTAGCCAGGCGTGGTGGCGCGCACCTGTAATCTCAGCTACTCAGGAGGCTGAGGCAGGAGAATCGCTTGA
ACCCGGGAGGCAGAGGTTGCAATAAGCCGAGATAGTGCCACTGCACTCCAACCTGGGTGACAGAGTGAGACTCCATATCAATAAATAAATAAAG
TAAGTAAATAAAATACTGTTCATGATATTCTGCTCCCATGAGAAGCTTGCCGTTCCTCCTCATTGCAGCCCTATGACTTCCCGTCTGGCGCCTT
CAGTCGCTGCTTTGTCTCCTCCTTGGATGCCTTTTCTTTTGTCCTCTCATCAAAACCAACTCATCCGTGAGGCTCCAGCTTGAGCTCTACCTCC
TCTATGAAGCCCCCGAACCTTTCCCACCGACACAGACCTCTGCCTTCTTTGATCATCTGCTGGACTTCAGTTTAATGCTTTAATGATTCCACTT
GTATCGGTCCTGTCTATCCACCCGGTTTAGGTATCTTTGAAGATAGGAGCTACCTACTTTTTAGGTCTTTTGCATCCTCCACACTCCTCATCTA
AAGCTGCGTCCACTCTGAGTCACCTTCCTAACCTGCCAGGCAGGGTCTTTTTGTTTTTTAAACTTTAAGTAGGCTAGGCCGGGCGCAGTGGCTC
ATGTATGTAATCCCAGCCCTTTGGGACTCTGAGGCAGGCAGATCACAAGGTCAGAAGATCGAGACCATCCTGGCTAACACGGTGAACCCCTGTC
TCTACTAAAAAATATAAAAAATTAGCCGGCCGTGGTGGCAGGAGCCTGTAGTCCCAGCTACTCCGGAGGCTGAGGCAGGAGAGTGGCGGGCACC
CGGGAGGCGGAGCTTGCAGTGAGCCCAGATAGTGCCACTGCACTCCACCCTGAGAGACACACCCAGACTCTCTCTCAAGAAAAAAAAAAAAAAA
CTTTCAGTAGGCTATGTGGAGGTCGAGTTAGATATCTTCGGTACAAGCCTTAGATTTCTTTTTCTGTTTTAAGGGAATTTTTTTCTTTTTCTCC
AATTTTGTTACTGTGAGTTCACCAAGGCATATTCAACAGCTCATTGCTGCCCTGCCTCTCCTGCACCATCCAACATCTCACTGCAGTATCCAGC
AACCCACAGGCCTGCTGGGCGTGGTGGACAGTCCCCAAGATCAGGTTATCCGTGTCAAAATATTGAGTGGTTGATGAGTGCTCCAAGTAGGCAC
TAGAGCCTGCTGAGAGCACTCAGAGGTTTCTGCACAAGCTCAGTGGATTTGAGCCCTCATCATTGTTCATTGCTATGTAAGTCAAGGGTTGGTC
ATAGGTCCTGCCTTGGACTGTCATTTCAGGAAGCAGGGCAGCTACAGCAGCAGTCCCCAACGTTTTTGGCACCAAGGATCCATTTTGTGGAAGA
CAATTTTTCCATAGATTGGGGGTAGGGGGATGGTTTCGGGATGAAACTGTTCTATCTCAGATCATCGGGGATTAGATTCTCTTAAGGAACGCAC
AACCTAGATCCCTGGCGTGTGCAATTCACAGTGGGGTTTCCGCTCCTGTGGGAATCTAATCCCTCTGCTGATCTCACAGGAAGCGGTAAAGCTC
TCTTCCCTGCTGCTCACTTCCTGCTGTGTGGCCCCTTTCCTAATAGGCCATGGACAGTTACCAGTCTAGAGGCTGGGGGGCTGGGGACCTCTGA
ACTACAGGCTTCTCCACCTGCACAAGTTCTGAGAAGTTAGACTATCCAGAGCAAGAGGGGCAGGAACTTCCTGGTCTCACTGAGAATCTTCGGC
CAATCTCAACTCCCAATGGGCATATGACTCACACGGTGATCATCTTAAAATGCAGATTCTGAGTCAGTGATTTGAAAGGGACCTGAAATCTTGC
AATTGTAACAAGCTCCCAGATGATACCAATGGCGATGTTGATGCTGCCTGTCTGTGGACCACACTTTGAGTAGCAAGGGTCTACAATATGGACA
TTTCCTTTTCCAGTGAACTTTAGCAAGTGAGGAGCATTTCAAGGAAGACACCTCCCATGCACACTGTCATTAGTTGCAGCATCTCCTGTAGCAA
TTGGGCTAAATCTGTCTTTAATTCTTTTTCAAACAGCATATCCAAGTTTTGCAATCATAAGCACCAGCCAGGAGAATATATATTCCATGCAGAA
AATGATGATGCCCAATTTACCAAAATGTTCACGCTGAATATAAGAAGTAAGTTAAATTGCCCAGAATTTGATTTTTATTTTTTAATTCCTTAAT
TTTTTATTAGTCTTAACTAGGAAAAGTTAAGTAATGGCTTAGAAGGCCTACTACAAAACTGTTTTTGTACGCTGAAGTGTTTTGAAACTTTACT
CTCATATCTACTGGCATTGATCATATATCTCTTTATTTTTTATGTTATTTTCTTCTGTTTCATTTTTAAGACCAGGTCTCACTCTACCACGCAC
GCTGGGGTGCCATGGCACAATCATAGATCCCTGTAGCTTCAAACTCCTGGGCTCAAGTGATCCTCCTAAGCCTCCCGAGTAGCTGGGACTACAG
GCACGCGCCACCATGCTCAGCTAATTTTAAAACCATTTTTGTAGAGAGGGGGCCTCCCTTTGTTGCCGAGGTTGGTCTCAAACTCCTGGCCTTA
AGGGATCCTCATACCTTGGCCTCCCAAAGTGCTAGGATTACAGGCGAGAGCCACTGCACCTCGCCATATATCTCTTTCTGTGTGTATCATCAGA
AGCTGAAAGACTTATTTCACATTGTCTCTGAATATGAATTAGTGTTATGACTTTATTGATTTTAGTATTTTGAAAGGTCATGTTAAGGAAAGGC
TGAAAGTCATTTTATCTCTCATTTATTTGTTTTTAATAATTTTCATTTCTTTGGCTTATAAAACAATGAGAAGTCCAACTTCTCCTGAAGCTAA
GAATCATTTTAAGGCATGCAAACAGGTGGACGTCTGTACCTCAGCTCTCTCCAGGGAGAGGCTGGCAATGGAAAAATGGTGTGTAGCAGAGAGG
TTGCATGGCAAGGAGGTTGCATGGCAAGGAGGAAAGAAGAAATTACCAGAGGTCTGGAGGCATCCTAAATGAGAAATCCTCCCAGAAGCACAAA
CTGCCCTGGAGTAAATGGGTAGCCTGTACTGTAGGCAACTTTGAAACTATTTCCAGAAAGTACCATACTCCATGAGCTTATTTTATTTTATTAT
TTTCAGAGACAGGGTCTTGCTGTCTTGCCCAGGCTGGTCTTGAACTCCTTGGCCCAAGTGATCCTCCCACCTTGGCTTCACAAAGTATTGGTAT
TATAGGAGTCAACCACTATGCCCAGCCAGTGAGCTTATTTCACACGTTCTTTTCTACAGGGAAACCTCAAGTGCTCGCAGAAGCATCGGCAAGT
CAGGCGTCCTGTTTCTCGGATGGATACCCATTACCATCTTGGACCTGGAAGAAGTGTTCAGACAAGTCTCCCAAGTAATAAGGACATTGATGCC
TCTTATAATGGAAGAACATAAAACTCTCTGACCTATTGACTCTTTCATGAATACGGTTTAGGAGGAAATGATGAAATATTTAAAATGCTAACCT
TCTCAATCAACCATAACTCTCAATAGTCATAGACTGTTTCTGATAACTTATACTGGATGAATCTCTTTTAGAACTCGCTGCAGAAATCCTAACT
TCCAATCTGATGACAGCACACCTGTCTAAATTCTTTGCCATTTTTTTTTTCAGCTGCACAGAAGAGATCACAGAAGGAGTCTGGAATAGAAAGG
CTAACAGAAAAGTGTTTGGACAGTGGGTGTCGAGCAGTACTCTAAACATGAGTGAAGCCATAAAAGGGTTCCTGGTCAAGTGCTGTGCATACAA
TTCCCTTGGCACATCTTGTGAGACGATCCTTTTAAACTCTCCAGGTACAACAGTTGTTCTTGTGATATTCCACCATCAGGAATTCCCCACTAGA
AGTTAGTCCCCATCGCTGACTTTTACTTGGGGAATTTTACAGGAGAGTATAGGGTCAGTGTGTGAGAAAAGGTTTCTTGAGTCAGGCAAGAATC
CCCAGAGAGACTGTTTCACAGGTTGGCGCGGAGTCCTCCCTGTGCCCTTGTGACTCATGTGTCACAGTGACGGGGAGAAGCAGAAACTGCCGGT
GTGGTGAATCTCCATGGAAAGATCTAACTCTAGAGAAACAGATTTTGTCACTTGGAGTCTCTGTCATTTTCCCTTTTGATTAGCCTCCTTATTT
GCCCTCAGCTTGCTGTTTTATAATTTTTGGTTTGCTTTTTAGAGCCCTCCTAAGTTAAACAGTTGTGAAACCTGGCTGCCCACCCTAGTAGTTA
GGATCACCAACCTCACAAAACTGACTTCTCCACTTTCATGTGCGACCTTCATAAAAGGGATATGAACTTCTTTTTTCTCTGACTTGTACATCCT
GTAGGTTTAAATCCATGCATTTTTTTTTTTTTGAGATGGAGTCTCGCTCTGTCACCCAGGCTGCAGTGCAGTGGCGTGATCTTGGCTCACCTCT
GCCAGGGTTCAAGCGATTCTCCTGCCTCAGCCTCCCGAGCAGCTGGGATTACAGGCACCTGCCACCACGCCCAGCTAATTTTTGAATTTTTTTA
GAGACGGGATTTCACTGTGTTGCCTGGGCTGGTGTCTAATTCTTCGACTCAAGTGATCCTCCCATCTTGGCCTCCCAAAGTGCTCCGACTACAG
GCGTGAGCCATCGTGCCTGGCCCAGATGTAATTCTTGTAAATTTACCCTAGTAAATGGTTTTAAAGAATGGATTGAGAATAATCCGCAATTTTC
TAGGGAGGAAATGTATCCTTTACAGGGACATTCCCTGATTGTCTGTGGGGAGTAGAGTATATGTAGAGTGGTTGTTAGGACTGAAAATGATTAT
TACTGAAACAGGATGTGAGAGATTATAATGAGTTGTCCACTATTTATAATGTCACACAGGAATTCTGTTTCATCGCTGAGTGACACTCTTTTGT
TGCAGGCCCCTTCCCTTTCATCCAAGACAACATCTCATTCTATGCAACAATTGGTGTTTGTCTCCTCTTCATTGTCGTTTTAACCCTGCTAATT
TGTCACAAGTACAAAAAGGTAAAAGCAAAGGTAAAAATTCATTATTCTTTCCTCTATCTGCAGAACTGCCTATTCCTAACTGACTCATCATTTC
ATCTCTGAAGCAATTTAGGTATGAAAGCCAGCTACAGATGGTACAGGTGACCCGCTCCTCAGATAATGAGTACTTCTACGTTGATTTCAGAGAA
TATGAATATGATCTCAAATGGGAGTTTCCAAGAGAAAATTTAGAGTTTGGTAAGAATGGAATGTGCCAAATGTTTCTGCAGCATTTCTTTTCCA
TTGGAAAATCTTTAAAATGCACGTACTCACCATTTGTCTTTGCAGGGAAGGTACTAGGATCAGGTGCTTTTGGAAAAGTGATGAACGCAACAGC
TTATGGAATTAGCAAAACAGGAGTCTCAATCCAGGTTGCCGTCAAAATGCTGAAAGGTACAGTATAGTGGAAGGACAGCAACAAAGATGCCCAA
AAATGGGAGGCACAGTTTCCCACCCATGCCTTCTTCTCTTTTCCATCCTTTTAATGGTTACTGTTTGCCTTGTTTCAAGGCTAAAAATGCAGTG
GATTGGGGTGTCAACCCAGTCATGAATACAAAATTCAAGTCATAAATACAAAACTCCACCTCTTCTTCCACTTCTCTCTTTGTTTATTTTTATT
TTCTTTTATTTTTTTGAGACAGGGTCTCACTCTGTCACCTAGGCTGCAGTACAGTAGTGTGATCATGGCTCACTGCAGCCTCCACCCCCTGGGC
TCAGGCTCCTCAGCAGCTGGGGCTACAGGCGTGCACCACCACGCTCAGCTAAGTTTTTTATTTTTGGTAGAGACGGGGTTTCACCATGTTGCCT
AGGCTGATCTGGGACTCCTGAGCTCAAACTATCTGCCCGCCTCGGCCTCCCAAAGTGTTGGGATTACAGGCGTGAGCCACTGTGCCAGGCCTAT
TTAGTTTTTATAGGCTTTGATATTTGGGGATACAGTTGCCACAGGCAGAAGTTCTTTCTTACATTTGTGTTGTTTTATTTTGTTTTATTTTTAG
AGACAGGATCTTGCTCTGTTACCCAGGCTAGAGTGCTGTGATCATAGCTCACTGCAGCCTCGACCTCCTTGGCTCTAGCAATGCTCTCACCTCA
GCCTCACAGGAGTGTGCCAAGACAATTGGCTAATTTTTTTTTTTTTTGGTACATACGGTGTCTCACCATATTGCCCAGGATCGTTGCAAACTCC
TGGCTCAAGTGAGGCTGCCTTGGCCTCCTAAAGTGCTGGGATTACAGGTGTGGGTTGAAACAGACTCTGGGTAGGAGAGAAAACTGGGGCCCTT
TGTATCCTCACTTCGTTGCTCCATCCATTCCGATCTGGCGTCCACCCCCATTGCTGGCCTCACCGTTACCACCGCTGACCCAGCAAAAGCATCT
CCAGCTGGCTCTCTTGCTAAGGTCAACAGTGACTTCAAACCAAACCAAGCCTAATGGATATTTTTCAGTCCTCATTACTTGATTCTTCAGCATG
AATTGATGCTGTTGATTCTTCTCTCCATCCAGAAGCTTTCTTTCTTTGGGTTTTGTGTCACCATATTCTGGTTTTCCTCCCACCCCTCTCCCCT
TCTCAGTCCTTTGTGCCCTCATCTTCTTCCATCCACCTTTTGTTGTTGTGGCTGATTTTGAAACTCAGTCCTTTGTGGGCTCATCTTCTTCCAT
CCAGCTTTTGTTGTTGTGGCTGATTTTGAAACACAGCCTTGCTCTGTTGCCCAGGCTGCAGTCCACTGGTGTGATCTCAGCTCACTGCAACCTC
CACTTCCCACGCTCAAGCCATCCTCCCACTTCAGGCCCCTGAGTAGGGGGGACTACAGGCATGCGCCACCATGCCTGGCTAATTTTTGTATTTT
TGGTAGAGATGGGGCTTCCCCATGTTGCCCAGGCTCCATCTTGCTTTTTATGGGGCATCCCTGTCCGCTTGCTCCTGGACCGCCTTGCGTTTTC
GCTCCACACACTCAACACAGTCCATCCCCACTACACCCGTGATGTCCTCCCATACACCAAGGGCCCTCATGCTTGCGTCTCCTTTCGGAGCTTC
AGGTTCATATGTTAAATGTTTGGATGTCTCAAAGGTCCCTTTAACACAGTGTATGCAAGGTGAATTCCCCTAACCCTGGCCCTGCACCAGCAGT
CCTCTCTGTGACTGGTAATGAATGCCAGTGAATAAATAACCACATTCTATATAGTTGGGCACACTAGAAACCCGTGACTTTTCCTTGACACCTT
CCTCTTTCTAACACCCTGTGAGCTAATCCTATCAGGTTTCCGATTAGTGTCTCTTGATTCCATCCTCGTCCCTCCATCTCTATGACCACCACTC
TAGTTCAAGGCTCTCTCCTGGACAAAAGCTTCCAAAGCAGGTTCCTGTATCCACTCTTGCCTCCCTTTGGCTTCTCCATACTGAGGTCCAAGTG
ACAGTGGCGAAAGGCTAAGCAGATCAAATGGCTTTCCTACTTCACATTTCCTCCACCACCCGCATGGCTGGGCCACCGCTGCCTCCCCACCTCT
TGCTCTCGGCACCCCAGAGCGCTGGCCTTCTTTCAGTTCCCCCAGCACTGTCCTCCCACCCACTGTGGGGCCTTTGCACAAGTGGATCCTTCTG
CTTCCCCTTCCCCTTTCATGTAAGTATCTCCCACTGCCCCCTCAGATCTCAGCTTAAACACCACTTCCTCTGTCTCACACCACAGACAATATTG
AGGCCCTTGCAATGTCCGACATTAGCTCTCTGCACGCTTTCCCCCGTAGAATCCATCACCTTTGTCATGACATTTTTTAACATCCAATTGTTGT
CCCATCTGTCCACTAGCCCCTGTGCCTCATGAGGGCACGGTCTGTGTCTGTGGTGCTGTGCTCTCTCCTTTCTCCACCTTTGCCCACTCTTTCA
CAACCCTCAGCACTGTTCTCCTCCCCTTCCACCATGAGTCACCTGGCCAGCTCCCATTTTTCTTCAGGACTCAGTTTAGACATTGAATGCTCCT
TTCTCCACCCCTCCAAGGCTAAGGTAGCTATCCCTTTTGACCCTACAGTACCCCTGCACTCCTTTGCCACGTTGCATTTTTTCTTTTTTTTTTG
ACACGGAGTCTCACTCTGTCGCCCAGGCTCGAGTGCAGTGGCACCATCTCAGCTCACTGTAAGCTCCGCCTCCTGGGTTCACGCCATTCTCCTG
CCTCAGCCTCCCTAGCAGCTGGGACTACAGGCACCCACCACCACGCCCGGCTAAATTTTTTTGTATTTTTAGTAGAGACAGAGTTTCACCGTGT
TAGCCAGGATGGTCTTGATCTCCTGACCTCGTGATCCGCCTGTCTCAGCCTCCCAAAGTGCTGGGATTACAGGCATGAGCCACCGTGCCCAGCC
CAACGTTGCATTTTTTCTTTTTCTTTCTTTTTTCTTTTTTTGAGATAGGGTCTCACTCTGTCACCCAGGCTGGAGTGCAGTGAAGTCATCATAG
CTCACGGCAAGCTTCAAGTCCTGGGCTCAAGTGATGGTCTGCAGCCTTTCCAGTAACTGGAACTACATGCAGGCACCACCAAGCCCAGCTAATT
TTTATTTTATTTTATTTTATTTATTTATGTATTTTTACTTTATTTTATTTTTAAGTAGAGACAAGCTCTTGCTATTTTGCCCGGGCCTTTCTTG
AACTTCTGGGTACCAGCAATTCTCCCGCTTGTCTCAGCCTCCCAAGATGTTGGGATTAGACGCGCAGGGACCCACGTCTTGCCTGCCGTATTGC
ATTTATGGCACTGTATTGTCATGGCTTATTTACTTCTCTGCCTTCTCCAGGAGACTCTAAGATCCAGGAAGACAAGGGTTGTGTCTTGTTTTTC
CTTCATTCCCCAGTGTCTAGGACATTGGCTCTTACAGAGTAGGCACTCAATAAATACTTGTTGAATGACTGAATGAAAGAGGTAATGGTACATG
AAAATGCTTAGAAGAAGCTAAGCATGCTAAAAAATGTAAGATATTATTTTAGCCATGAGGAATAGGACATGAACAAGTAAAGCCCCGAATCCCT
GTCCAGCGTGAAGCTGAAGACATGATGTATTAGGGGGTCCCAGTGCCCACAGGAAATCCCACCCCCAAGCTAAAATCTTGGAAACAAGGATACC
CCTGAGAGCTTGCCCAAACCAAAATAGACGATGTCAGCACCGGAGAAGCCACTATAAAGGTTAGGATGCCTTCCTGGACAAAATAATGGAACCT
GGCAATCGAACCAAATCTTCACTGAATGTAAAGAAGAGTTACTATATCTGGAAGAAAAAGTCTGTTCACAGAGGGGTGGTGCCTGGAGCCTGGA
GAGGGAAGTCCAGAGACCCTGCCCTTGCCCCTGTGGCTGCCCGGGCTGCCCTGACCAACTGCACATGAGCCGCAATGACGGCAGCCCTTGGATT
CCATAGCGACTTTCCTCACACTGTGCTTCTAAGTCAGCTTATAAGTACAGTTTGCTGAGCTATCTGGAATTTTCCCTAGAGAGAGAATGCATTG
GCCAACCAATATGTTCTAGCCTGCTTGGCAGTTTTATTTCTCAATGTCAATTAATGGGTCTCTTGAGCACCTGATAGTGGGAATTAGTCCTCTT
TGACCTTCTCCCATTGGACTTGCTGGCAAATCTAATGTTCTAGACCTCAGGGCCACAGGCTAGAGTCAACCATGCTCACGACACTGTTATCTCT
CTCTGACTACAACAGATCCAAGAGTTCAAACAAACAAACAAAAAGGACGTTTCTCCCTTCTGTTAACTTGTACTGAGGAGGAAAAGCATGTGTT
ATTATTTTGTTTCTTTGTTTTGAGGGCCAACTGTAAGGATTTTTATTTTGGTAGGAGTAGAGGAGTCCTGTTGAAAGGTGCATTACCAGATTCA
ACATTTATACTATAGTCTTTGTTTTGTTTTGTTTTGTTTTGTTTTTGAGACAGAGTCTCGCTCTGTCACCCAGGCTGGAGTGGTGCAGGGGCTC
AATCTCGGCTCACTGCAACCTCTGCCTCCCCAGTTCAAGGGATTCTTTTGCCTCATCCTCCTGAGTAGCTGGAATTACAGGTACGCACCGGCAC
GCCCGCCTAATTTTTTGTATGTTTAGTAGAGACAGGGTTTCACCATGTTGGCCAGCTTGGTCTTCAACTCCTGACCTTAAGTGATCCACCCGCC
TTGGCCTCCCAAAGTGTTGGGATTACAGGTGTGACCCACTGCGCCCAGCCCTATACTACAGTCTGAATTCTGCCTTAGGCTGGGCATCGTGGCT
CATGCCTGTATTCCCAGCACTTTGGGAGGCGGAGTTGGGAGGATTGCTTGAATCTAGGAGTTCAAGACCAGCCTGGGCAACATGGTGAGACCCC
ATCTCTACAAAAAATATAAAAATTAGCTGGGCATCATGGCCTGTGCTTGTACTACCAGTAACTTGGAGGGCTGAGGTAGCAGCATCACTTGAGC
CCAGGAGGCAGAGGCTACAGTAAGCTGAGATCATGTCACTACACTCCAGCCTGGGTGACAGAGTGAGATACTGTGTCAAAAAAAAAAGAAAGAA
AGAAAAGGAAAAAAAAAAAAAGAAAAAGGAGAAATTCTGTCTTAAAACACAGGATATTGTTTGTTTGTTTATTTATTTATTTATTTTAAGATGG
AGTTTCACTCTTGTTGCCCAGGCTGGAGTGCAATGGTGCAATCTCAGCTAATTGCAACCTCTGCCTCCCACATTCAAGCGATTCTCCTGCCTCA
TCCTCCCAAGTAGCTGGGATTACAGGTGCCTGCCACCACGCCTGGCTAATTTTTGTATTTTTAGTAGAGACGAGGTTTCACCATATTGGCCAGG
CCAGTCTTGAACTTCTCACCTCAAGTGATCCACCCACCTCGGCCTCCCAAAGTGCTGGGATTACAGGCATGACCCACCATGCCTGGCTTCTCTC
ATAATTTTGAATTTAAATAAATAATGCAGATTGACTCTGAGCTGAGAAAAATTTTATTTAATGACATAAAATTCGTATTTTACTTGAGTTTTAC
ATTTTTAATGCTCCTTTCTTTGACAGAAAAAGCAGACAGCTCTGAAAGAGAGGCACTCATGTCAGAACTCAAGATGATGACCCAGCTGGGAAGC
CACGAGAATATTGTGAACCTGCTGGGGGCGTGCACACTGTCAGGTAACCCACTTCCACGAAAATCACCTCATCAAAAAGACTGTAGCTTGATGA
CAAAGAGGATGTTTGCTCTCTCTCTCTCTCTCTCTTTTTAATTCTTTGTCTTTTCTCAGTCACAGTGTGAACTCTCAAACCCAAGTGGAATTAG
ACACTGAGCACTCTATAAATCAGAGGGGTCAAAATAAAGAATGCCTCATTCATCTGCTTTATCGTGATATATTGCATAATTCTCACCTGCTTAA
AAACAACAACAACAACAACATTGCAAGTACATTTGGTGGTGTCTGTGCTTCTCTCCCAGCTTGGAAGCTGGTGCACAGGCGTGGTGAGACTGGT
TACAGAGCCTGGCCACTGCCCCAGTTGGGCTGCCGTCAAATACGGCACAGCCTAGCTAGGATATCAGACTTTACTTTCCCAGAATTCTCTGGGG
CATGCCCTCTGATCTTCTCTCTTCTGCCTAGAAATACCCAAATGATGTGCCCAAGGTTCATTCTTTCTGCAGCAGCAGCCCTGGGGCTGGTACT
AAAAGGAAAAAGAGGAAAGTGAATCTGAGAGAGGACTGGGAATATATTTCTCCCTCCTCAAGCAAACAGCAAATCAGCATATAGTTACCAAGTG
TTCACCTATAATACACAGCTAGAACATTACTTTCAGTTGCCAACTGGAGAATCCAGGAGGCCAGTGGTATTGGTGAGGCCACACATACAAAAAA
GGGCCACAGAGAATGTCAAGAAGATGCTGCTTTCCAAAGAAATTTGAGCAAAAGCTGAAACTGAATTTAAAGTATAAGAAGAGCTAGGCTCAGA
AAAAGTTGTACTGTCCCCAAGTCAGCAGAGAACCAAGCCCTCCTAAGAGTATGTTGTCTGCTACATAGACTTCTGAAATAACAGTTTGCTTTGT
GTATGCCTATAATTGAAACTGTAACTATTTCAGGACCAATTTACTTGATTTTTGAATACTGTTGCTATGGTGATCTTCTCAACTATCTAAGAAG
TAAAAGAGAAAAATTTCACAGGACTTGGACAGAGATTTTCAAGCAACACAATTTCAGTTTTTACCCCACTTTCCAATCACATCCAAATTCCAGG
TAAGAGGCTGGGTCAGGGTTTCGTAATTACACATCATAGAACGTAGGCAAAGTGTGCTGTTCAAACACACTTCGTTGACCAGTTTCTAAAGGCA
CCGTGAAGTCTGTGGGTCCTGCAACCTAGAGATTTCCTGAATCTTTGCTATAGTTTCCCTGTTGACATGGAACGAGTCCTGTCCCTTTCCTTCA
GTACACCTACTATAGCTGAGCTAGTCTCTCTCCACCCATGCCTGCATCACACCATAGACGCCTATTTGGTTTCGCCAAAGGTGCATTATTCGGT
CTTTCACGACACCTGGTTCCCTCACAGGTATTGGTAGAAAGAATGAAGACTTTTTCCAAAGTCCTTAACATATGCAAAGAGAGCAGCTCGGGTC
CTGGGACTATTGCGCAAAAGAACGCTGAACTCAAATTGGATTCTAAGATATTAGGATGGATTGATCGACACGTGATGGGGACACAGTAAAGTAG
CACTAACCTTGTCTCTGCTCTGTTTATCCTCCGCATCCATCGGCATCCTGAGAGATGCTTCGTCCTGCAGGGATTTATTGCTTTACCCGGCATA
AATGAGGGCTGTAGGAGTTTGCTGGATCTCCTCCTCATCCTTGAACTTTCTTGCTAATTACTTGAGCATCATGGCTTTGTACCACCTTTCTCTG
AATGGATTCCCAGGCATTCTCATGGAGGCATTCGTTGAGCCTTGGAGCATTCTTTTTTTTTTTTTTTTTTTTTCAGACAGAGTTTCGCTCTGTT
GCCTAGGCTGGAGTGGTGCAATGGTGTGATCTCGGCTCACTGCAACCTCCACATCCCAGGTTCAAGCGATTCTCTTGCCTCAGCCTCCCGAGTA
GCTGAGATTACAGGCATGTGCCACCACGCCCAGCTAATTTTGTATTTTTAGTGGAGACAGGGTTTCACCATGTTGGTCAGGCTGCTGTTGAACT
CCTGACCTCAGTTGATCCACCCACCTAGGCCCCGCAAAGTGCTGGAATTATAGGCATGAGCCACCGCACCTGGCTGACCCTTCGCGCGTTCTTA
TTTCCCTCCCTGTATGGAAGTTCTCCCTTATTCTGAAGGGTATTTGTTTATTGGAGACTTTTTTCTTTACCAGGCGCTTTTTAGGGATACATTT
GAGAGTCCCGGCTTTGTTTTCTTAGAATGTAAGTTTTAAGTGCAGGCCTCTGGCCTTCCAGGTCTGAGACACAGACCCAGTGTCTTAAAGCCAC
CAGGCCCCACGGACATCCCCATGCACGTCCGTGTGCAGCTGCCCTTAACCTATGCATGCCCGTCACACCGTCCTCTCCTTTACATTCATTTCCC
AGCTCATCGTCTAGAAATGAGAACTCTATAAAGCCACTCATATTCTGGAACAGAGGGGCTATCCTACTGAGTCCGATGTGTGTGACCAGTATCC
ACTGGTCAGTACTTAGCCCCTTTGATTCGTTCTGTTCACTGCAATATTAAATACACTGAAATTATTCCTTTTGGAATATTTGTGGTCTGAAAGC
ATGAAGCCATTTCCTAGATAAATGTAAATTGTAAATTGGGTTAAACTCGTATATTACAATAAATTGAATCGTGATCTACTTATAATTCCTACAG
AGCTCCTCAGGGAAGAATTTTTATATACCTTAAAAGAGATTTGTATTATTGCTATTTATTAATTTCACAGTAAAAAGAAATGAGCTTTACAAAG
GCAAACTGCAAAAAAGAAGGATGCTCAAACGCTTACGGGACTCTCGGGAACATCTGTATTATGTGAGGGAAAGTCGCCTGAGTTCAGAAACCAA
AGAATGAGATCGATGTGAGTCCTGGTTCCACTCGAATCATATTTATCTCACTGTGCCTAATTTCTCTTTTATAAAATGCCACCCATCTCATAGG
TTGTGCCAACGATTGAGTGAGGTCCTTTTTGAACTGTAAGATTCTATATCGAAAACAGGGATTACTTTTATCTTAGACTCGCAAGCCCAGTTAC
ACAGATGTTTTTAATGTACACAAAAAATGGACAAATGGATCGTCTGCTTTTAATTTAAAAGTCAATTTATAACTGGAATCCATTTATTTGATGA
ATTACATTTTTAAAATAAAATGTCTGCTGAGAAGAATAATGTAAATAATGAAAAAACAAAATTTTTTAATAGCATGCCTGGTTCAAGAGAAGTT
CAGATACACCCGGACTCGGATCAAATCTCAGGGCTTCATGGGAATTCATTTCACTCTGAAGGTAATATTTTATTCTAAGTAGTAGCACTTTAAA
ATGGAAATGAATGCAAAAGTTTTATGTTAGCTATTTTTTAAAACTCTGTAAACACAAAAGCATTTTAAAATTTGCTAATTGTCAGATGATAGCT
AAAAGCCAGTTTGTATACTGTAGATTTTATGTCAACATTCAACTAATAAACACTGAGGACTATTTTATTTGGTCAGATTATTCATTTAAAGTCA
TATATTAATTACAGCCATCTTATTACCTGACAAGTGGAAGAGATAATGAGTTCCGAAGTTACATAGAATTTGGATTAACTTCTGCTTACTTTGC
TTTCTGACTGGATGACTTTGGACAAGTTATGTGACCACCAGAATCACAATTTTCTTGTTTATAAAATAGGCCTAACACTACCTTCCTCACAGGA
CTGTTAGAAGGAGTAAATGAGGGGCTGGCCGTGGTGGCTCACGCCTGTAATCACGCCTTGGGAGGCCGAGGCAGGCAGGTCACCTGACGTCAGG
AGTTCAAGACCAGCCTGGCCCAATATCGAGAAACCTTGTCTCTACTAAAAATACAAAAATTAGCTGGGTATGGTGGTGCACGCCTGCAATCCCA
GCTACTTGGGAGGCTGAGGCAGGAGGATTGCTTGAACCCAAGAGGCAGAGGTTGCAGTGAGCTGAGATTGCACCACTGCATTCCAACCTGGGCA
ACAGAGTGAGACTTCAACTCAAAAAAAAAAAAGAAAGAAAGAAAAAAAAAGGAAGGAGTAAATGGAAGAATATAAAATGCACAATATAGTTTAT
AAAATAGGATAGATGGGCCGGGCACAGTGGCTCACGCCTGTAATCCCACCACTTTGCGAGGCTCAGGTGGGTGGATCACGAGGTCACGACATCA
AGACCATCCTGGCTAACACGGTGAAACCCTTCTCTACTAAAAATACAAAAAATTAGCATGAGCCTGTAGTCCCAGCTACTCGGGAAGCTGAGGC
AGGAGAATCCAGTTGCAGCAGGCCAACATCGTGTCACTGCACTCCAGCCTCGGCCACAGAGCGAGACTCCTTCTCAAAAAAAAAAGCGATAGAC
AATTAATATTAATTCCTTTCCCACTGTGACAGCCCTACCTAGTGCAAAAGAGAAGTATCTAAAACTCCTGACTAATCAATCAGGAGGTCATCGG
TCATCAAAGCAGTTCCAAAGTTTATCTCTAACCAAAACAAGTCTCCAGTGACCCTTCATAGGCAGCATAAACAGGTCATCTCTAATGCTGCCGG
GAAGATAATGCGATAGCTTCATGTCTTAGCACCTCACCTAACTCTGGCTTGAATAACCTTCCCATAGAGATTTAATAAGGCAACTTCTTAGAGT
TTTGGCAAAAGTGAAGACTGGCTCTATTTTACAGATGAAATTGAATATGATAACCAAAAAAGGCTGGAAGAAGAGGAGGACTTGAATGTGCTTA
CATTTGAAGATCTTCTTTGCTTTGCATATCAAGTTGCCAAAGGAATGGAATTTCTCGAATTTAAGTCGGTATGCTCCTTTTAAAGAAAAAACAA
ACCAAAAAAAAGATTTGAAAAGATGTGCTTATATATGTTTTCTTGTTTATTTTAACTTGTATCCCTTAAAGTCTGCCTTTTCTCCCCTGTTCAC
TGTTGGAGTCACCTAGCGTGTTACCTTGGCTCTTGGCTAATTGTAGTAGCATAAAGGCCTGGGATCCCTTGTCAGGTCACGGCTAGCACAGCCC
TCCCAGGCTGCACAAAGTCCCCTGAAACTTCCAAGGCTGAAGGTCGAGATAGGAAGTCCTCAGAAATCCTTCCTCAAAACAAAGCGTAGCATTG
TCAGTCCCTTTCAGAACGGCTGTTCAGGTTGTGCCCTGCATGTGGGCATTCAACCCAGGGGATGAGAGGAGGCTGAAACCGAGCCAGCCACCCT
GAGTGTCTTTGTCTAATTCTCACCCTGCGTGCTGTGGGCTGGCAGCAGTCCTGCAGGCTCACTGCCTCCTCTCTTGCTAACCCCAGATCCATTC
GTTGGTAAGGCCCTGGAGCCTGCCGTGAACCATGTCTCTACCCCAATTTTGCACTCTTTTTGAACTGCCTTTCCACCCGCTGTTCTCTGTGATT
TCAAATGTCTCCTGCCTATAAGCAGATCATAGCCCACTCTCTGGCTACCTTCCTTCCTCGTTTGCTTGCCTTTCATTACCTTCTCTTTATTTCC
CAAAATTCTTCCCCCTCATTTTTCTTGCAGTCTTCTAATCTCCTCCTCCTCTTCTCCTCCACTTCCTTCCCCTTGCCTCTTCTCTCCTGTTTTC
TCTCTGAATTTGGAAAGACTACTTTCAATTGTATTTTTTTGTTTTGTTTTGTTTTGTTTTTGGAGACAGTCTCGCTCTGTTGCCCAGGCTGGAG
TCCACTGGCGTGATCTTCGCTCACTACAACCTCTCCTTCCTGGGTTCAAGCAATTCTCATGCCTCGGCCTTCCGTGTAGCTGGGACCACGGGTG
CATACCAGCATGCCTGGCTAATTTTTTGTATTTTAATAGAGATGGGAATTCACCATGTTGCCCAGGCTCGTCTTGAAATCCTGAGCTCAAGCAA
TCCACCTGCCTCAGCCTCCCAAAGTGCTGGGATTACAGGCGTCAGCCACCACACTCGGCCTTTTTTCTTTAAAAAATTACTGCCTCAGCCAGAT
GTTCATGGTGTGTCTCTGCAGTTCCAGCTACTTCGGAGGCCAAGGTGGGAGGATTGCTTGAGCCCAGGAGTTGGAAACTGCAGTGAGCTAGGAT
CGCACCACTGCACTCCATCCTGGGTGACAGAGCAAGTCTCTTTCTCTAAAAAATAAAAAATAGCCAGCCGCGCGTGGTAGCTCACGCCTGTAAT
CCCAACACTTTGGGAGGCCGAGGTGGGTGAATCACGAGGTCAGGAGATCGAGACCATCCTGGCCAACACGGTGAAACCCCGTCTCTATACAAAA
AAAATTAGCCGAGTGTGGTCGCGGCCGCCTGTAGTCCCAGCTACTTGGGAGGCTGAGGCAGGAGAATGGCATGAACCTGGGAGGCGGAGCTTGC
AGTGAGCCAAGATGGCGCCACTGCACTCCAGCCTGGGCGACAGAGTGAGACTCTGTCTCATAAAATATAATATTTAATTAAAAATAATAAATAG
GCTGGGCACAGTGGTGTACGTCTGTAATCCCAGTGCTTTTGGAGGGAAGCCCAAGAGTTCCAGGCTGCAATGAGCTATGACTATACCACTGCCC
TCCAGTCTGTCTATTCAAAATAATAATGAAATAAGCTTAAAACCAATAATAATACAAGTTTTAAAAAATTACTGTCACAGTAATATTTCCACAC
CTCTGAACTGCGACGCACATTTTTCTCTTCATTTTCCCCACCTGCTGTTTGGATTCTTCTTGCTCACGCATCTTGCCCACCTGAGTCTTTCTCT
ATTTCACTTATACAACTACAATCCTGCTTTCACAAGCATTTTGTTTTTGTTTTTGAGACAATGTCTTGCTCTGTTGCCCAGGCTGGAGTGCAGG
GGTGTGATCTCAGCTCACTGCATCCTCTGCCTCCCAGGTTCAAGCGATCCTCCTGCCTTGGCTTCCCAAGTAGCTGGGGTTACAGGTGCACACC
ACCACACCTGGCTAATTTTTTTGTATTTTTGTAGGGATAAGGTTTTGCTATGTTGGTCAGACTGGTCTCAAACTCCTGACTCAAGTGATTTGCC
CGCCTCGGCTTCCCAAAGTGCTCGGATTACAGGCGTGAGCCACTGTACCTGGCCCACGAACATCTTTATAGAGGTGGGAGAACACCTACTTAGA
TTGTCTGGCCTGGTTTGCTGCCCATAGCTAAGTAAATACTCAAACTACTCCCAACTGGCAGTCAAGGGGCACTGCTTCCCCTTCAACCCCAGGC
TTCTAATAATATGAATTTTTGAAGAACATCCACTTTCTTACGCCTTCTATCCTTTTATCTTCACATCAGGCCTCCCCTTTGAATCATGCGTGTC
ACTGTCCCCTTCTTGTCTCTCACCCCCTCCCTGATATTCCTCTGGGTGCCCCTCACTTTCCTCTGTAGGCCCTTGACTTCATCAAACATTTGTT
GAGCCCCAGCTACATGCCAAGCACTGTACTAGATCCTGGAGATACAGAGTCACTTATGATATGACTCCTGGGCTCGAAGATCTTATGAATGATA
GGGGAGACAGAGACAAGCAGACATGCAAGTCCAAGATGACACTGAGCCCGGGGGTGGGTGGCTCAGCCCTGTAGTCCCAGCACTTTCCGACGTT
GAGACGGTGGATTGCTTGAGGCCAAGAGTTCAAGACCAGCCTGGCCAACATGGCAAAACCCCATCTCTACTAAGAATAGAAAAACAGCCAGGCA
CGGTGGTGTACGCCTATAATCCCAGCTACTTAGCAGGCTGAGGCATGAGAATCGCTTGAATTGGGAGGCAGAGGTTGCAGTGAGCAGAGATTGT
ACCGCTGCACTCCAGCCTGGGTGACAGAGTGAGACTTTCTCAAAAAATAAAAATAAAAAATTTTAAAAATTTAAAAAAAGACAAGACTGTAGCA
TGATAAGTCTTCTGACTGAAGTAAGTTCTGGCAACCCAGGGGAGGGATCTGCACAGCAGACCTGGTTGGGGGAAGGCCACATGCAGTGGAAGAG
GCTTTCTGAGGGAGTGCGTGCTTCATGCTTGGACTAATGGAGATTTTTGAAGGAAAAGGAAAACCAGCAGGGAATGTGGAATAGGATAGGAAGC
ATGGAATCAGGAGGAGTTTTTTTTGGAGAAAGGTAATGAGTTGGACATACTGAATGTGAGGTGCCTTGAGCAACCAAGTAGAGCGTGGTCTAGC
AGACACTTCCACACTTCTCCTGTCTGAACCTCAAGAGAAATGTTTGGGTTGGAAATACCGATGTGCGCAATGGTGGTTGCATCCCTAGGTTTGC
ATGAAATGCCTTGGGTGAGGGTGTGCGTTGTGAGGATGCAGCCCTCAGCAGTCACAGTACGCTCGTGCACAAAGCCCCTGAGGGAGACAGGAGC
CGGCAGAGCAGTGGGGAGAAAGCAGGAGTGAACCATGCGCAGGGTTCCCAGAGGGAGGACCAGAAGCGGGAGGGGCTGGCCAACAGCAGCAGAC
ACCACAGGGAGGTCCCGTCAGATCGAGACTGTGTGGGTGCCAAGCCAGTCAGTGCAGGCCTCGGCCAGAGCAGCTTGGCCACACAGGCAGGAGG
AGGAGGCAGGTTGCAGACCTGGGGTAAAGTGGGCAAAGTGCAGGTAGATGGCACTGAAAAAACTGGGTATGGAGGAGGGAGCTCTAGAGGCGCT
GTGGCACAGGGCTGCCAGTGTTCATGGTGCTGCTTTTTATTTTTTAAAGATGAAAGAGTGTACATTGGACCTTTGCTACTAGTGCCCTGTTCCC
CTTCAGATCTTCCTTTGTAAAGTTCATTTTCTTGTCAGACCCCTTTGATTAACATCACGACATCTCTCAGCTCAGGGACTCCTTCTAATTCCAG
TTCAGTAAACCTCTCCAAACTCATTTTTAGCTTTTTGTTTTTCCGATTATTTCCCTCGATATTACTCCATTTATTCCTAAAGCTATTTTCCAGT
CAGAAAAGAGATGCTGGCCAGGCGCGGTGGCTCACCCCTGCAATCCCAGCATTTTGGGACGCCGAGGTGGGTGGATCACCTGAGGTCAGGAGTT
CGAGACCAGCCTGACCAACATGGCGAAACCCCGTCTCTACTAAAAACACAAAATTAGCTGGGCCTGCTGGCGCGTGCCTGTAATCCCAGCAACT
CGGGAGGCTGAGGCAGGAGAATCGCTTGAACCTGGGACCCAGAGGTTGCAGTGAGCCGAGATCACGCCATTGCACTCCAGCCTGGGGAACAAGA
GTGAAACTCCGTCTCAAAAAAAAAAAAAAAAAGAAAAGAGATGCTATTTCTCACACCCACAATAAGAGTACTGGAGGACAGCTCTGAGATTCCC
TCTGAAGGCTCAGGGGAGGGCCCCTGTTGGGGAGCCTCTCACTTTTGCTCGGAATCTGCAAAACATCTGCTCCTCTTTGGCCTCTGGGGATGCT
TCAGAAGCCGCACAAACAACTGCAGCCACCATAGCTGCAGAATTAGGTCTCCTATATTCACTTTGTTTGTTGCACATCATCATGGCCGCTCACG
GCACAGCCCAGTAAAGATAAGAGGCCTTCCATCACCGGTACCTCCTACTGAAGTTGAGTCTAGAAGAAAGATTGCACTCCAGGATAATACACAT
CACAGTAAATAACACTCTGGTGTCATTCTTGACAGTGTGTTCACAGAGACCTGGCCGCCAGGAACGTGCTTGTCACCCACGGGAAAGTGGTGAA
GATATCTGACTTTGGATTGGCTCGACATATCATGACTGATTCCAACTATCTTCTCAGGGGCAATGTGAGGCTGCTATTTCCTACTTATTTTTAT
ACGGCTATTTTGTGTTGTGTCGTTATCATGGTAAACAACTGCACTCACTGTGGTGCATTTTTGATTTATGGTAACATCAAAAAACCCTCACAGC
AGTCTCCTTACTTATGCTTAAAAGGTTTTTCTGCAGCTTCAGGGAATCTTCCATCTATAATAAAAGCTGAGCAAAATAGCATCACATCTGTCCT
TGGGCTAGCTAGTCACAGTAGATCAGTTAGGGGCCTTTGACTGCAATCCTCACTTTAAAAAACCCAAAATCTCAATACTTGCACTTAGTTCAGT
TATCCTAGCTATAAAGTCATGATTGAGTTATGTTTTCAATTATGTCAAAAGCTAATAGTCACAATAAACCACTTTAAAACATATATAGCACATA
CCTATAAAATGTTAAAGCTAAATACTTAAATATCAATTAATGAATATTTATCATCCTTTTACTGTTAACACAGGAAGATCTAAAACATGAGTGA
ATTGGCTGGGTGCAGTCGCTCATGCCTGTAATCCCAGCACTTTGGGAGGCCGAGGAGGGCAGATCATGAGGTCAGGAGTTCGATACCAGCCTGA
CTAATATGGTGAAACTCTGTCTCTACTGAAAATACAAAAAATTAGCTGGGCGTGGTGGCGTGTGCCTGTAATCCCAACTATTCGGGAGGCTGAG
GCAGGAGAATTGCTTGAACCCAGCAGGTGGAGGTTGCAGTGACCTGAGATCGCGCCACTGCACTCCACCCTGGGCAACAAGAGCAAAGCTCTGT
CTCAAAAAAAAAAAAAAAACAACAACAAAAAAAACAGTGTGAATTGACTTGGTAATGAAAAACTAAGCACAGATGTGTTTACAAAAAGAACTAT
TACAAGTAATAGATGACAAAATTGTGTAGTTGAAAATGTGTGTTTTTAAAGAATTACAATACCTGGCTGCGTGCTGGTGGCTCACGCCTTTAAT
CCAAGGCACTTTGAGAGGCCAAGGTGGGAGAATTGCTTGAGCCCCGGTTCAAGACCAGCCTATGGAACATGGTGAGACACTCAACTGTCTCTAT
AAAAAATAAAAAACTAGCTGAGTGTGGTGGCACACGCCTGTGGTCTCAGCTACTTGGGAGGCTGAAGTGGGAGGATCACTTGAGCCCAGGAGGT
CGAGGCTGCAGTGAGCTGTGATCACGCCACTGCACTCCAGCCTGGGCAACAGAATGAGACCCTTTTTTTTGAGACCCTGTCTCAAAAAAGAATT
CCCTACTTGTGAAATAATAATAATATTAAATGCTGACTTAGTGCTTACTATGTGCTAAACATCTAAGTGCTTTATGCATATTAATTCATTTATT
CCTCAGAGTACCCACTTGGAAACTGAGGCACAGAAAAGTAACTCGCCCATGGTTACACAGCATGTGGACTTGAACAGGAATAGTCTGAGCCCCG
AGTCTGTGCTAACCACTATGTTGTAGTGTTTCCCATGAGGGTTATGCACTTACCTCATAGCCTTCATGTGGCAAAAAGCCAATAAAACCAACGT
GAAAGCACCTTGTCCAGGGTGAACATCCAGTAAATGTGAAAAGTCAAATTAAACTGTATTCCCACTACAAACAAATAATCATCTTAGCTGATTT
ACGATCTACTCCTACTCATCATAGACTGCTCATATGTAGTCATTCAGTTTCTACCCCACCACCTGGTTGTCTCTTATGTGGGAAAGTGAATTCC
ATTTGGAACCACAATTAATTTCTTTAATTAATCAAATTAACCAGACTTGCCTGCATGAATCAATAGCTCATCAGTTTGCATACATTTAAAAGAG
GGTTTACTTGGCTGATTAAGCATGTGCTGATTAAACATGTGCTCTCAACGACTCGTCAACCCATGTAAAAGAAAATCAGTACAGCCAGGTGTGG
TGGCTCACAGCTGTAATCCCAGCACTTTGGCAGGCTGAGGCGGGTGGATCACGAGGTCAGGAGTTCAAGACCAGCCTGCCCAACATGGTGAAAT
CCCGTCTCTACGAAAAATACAAAAATTAGCTGGGCATGGTGGTGCGTACCTGTAATCCCAGCTACTCGGGAGGCTGAGGCAGGAGAACCACTTA
AACCAGGGAGTCTGAGGTTGCAGTAAGCTGAGATAGCGCCATTGCACTCCAGCCTGGGCAACAAGAGCAAAACTCCGTCTCAAAAAAAAAAGAG
AAAGAAAATCAGTACAAAGCTCTGCCCCAGTAACCTACCCATCTTGTCTCTTGATTCTAGTCTGAGACTATACTAAGTCAAAACTCCCTTTCAT
GCTAGTGGAAATTATTAAAGCCACAGAAGCCACACTGGTATCATTTCTGGAAGTGATGGCGTGGGCTCCCCTCCCGACATTATTGTGGATGACA
TTTCCACTAAAGGTCTTCCCAGAAATGCTGTAACAGAAGATCTCACTAATTCACTAGTACCCACTTACCCCTAGGCAACTGACTGGCTATTCCT
TAATGGAAAGTACCTCTTTCCAGTGGAAACATGGCAAGTTAAACTTACCCATTCCAAGCACAAGCCTTTGTTCGAGAGGAGTTGTAAAGAAATA
TGCCTTATGTTTTAGGCTAGTATTATTAGAATGTTAATTTGAAAGTGAAAACCTTTCACGTGAGGAATGTGATTTTCTCAATAAGGCATTAAGA
GTGGTCTTAGGAAGATCATGCATTTCATCTTAATACAAAACAAAAGAAGTCATGTCTGTATGTGGAAAATTCACCATCTTATCTCCTCTGGGTT
TTCCACAGGCCCGTCTGCCTGTAAAATGGATGGCCCCCGAAAGCCTGTTTGAAGGCATCTACACCATTAAGAGTGATGTCTGGTCATATGGAAT
ATTACTGTGGGAAATCTTCTCACTTGGTAAGTTGGGCAGCTGCTGGCCTCCCTTGGCTCCTGGCTTTGCATCCTCTGCTCTCTCGGTGCCCCCC
CACCCCTCCCCCCAACACTGATGGGTCTTACTTGTATTGCAGCCCATGACTTTATGTTTATTTGCTGGTGTCCTTTAGCCTCTCTTCCTCACTG
GTCTATAACCTGCTTGAGAGCGTCAACCGTGACTTCTTTTTCCCAGCCGCAGCCCGTGTGCCTCCAACAGTGTCTGGCACATGGCAGATCTCAA
TACGTGTTTAATTGAATGAATCAATAAACAAATGAAGAAATGCATGAATGGCTGCAATTGTCTAATGTTTTCCTCAGGTGTGAATCCTTACCCT
GGCATTCCGGTTGATGCTAACTTCTACAAACTGATTCAAAATGGATTTAAAATGGATCAGCCATTTTATGCTACAGAAGAAATGTAAGTTCAAG
TCAGACTGTAATTTGGTTGAAATCAGAAAGGTACAGTCTGTCTACTTCCAAATGCATGACCAAAAAAAGTCAAACTCCTTAGTGCAAAGCGTAT
AGGATTCTTTTTTTAAAAAATAGAACTCAGGTCTTGCTGTCTTGGCCAGGCTGGTCTCAAACTCCTGGCTTCAAGCAATCTTCCTGGCTCAGCC
TCTCAAAGTGCTGGGATTACAGGTGTGAGCCACCACACCTGGCTGTCTGATTCCATTTTAAAAAGAAAAGAAAACTCTTTGTTTTGGGAGTTTC
TGA
HUMAN SEQUENCE - mRNA
CCAGGCGGCATCCCAGGGCTGGGCCGGCGCCCTGGGGGACCCCGGGCTCCGGAGGCCATGCCGGCCTTCCCGCGCGACGCGGGCACCGTGCCGC
TGCTCGTTGTTTTTTCTGCAATGATATTTGCGACTATTACAAATCAAGATCTGCCTGTGATCAAGTGTGTTTTAATCAATCATAAGAACAATGA
TTCATCAGTGGGGAAGTCATCATCATATCCCATGGTATCAGAATCCCCGGAAGACCTCGGGTGTGCGTTGAGACCCCAGAGCTCAGGGACAGTG
TACGAACCTGCCGCTGTGGAACTGGATGTATCTGCTTCCATCACACTGCAAGTGCTGCTCGATGCCCCAGGGAACATTTCCTGTCTCTGGGTCT
TTAAGCACAGCTCCCTGAATTGCCAGCCACATTTTGATTTACAAAACACAGGAGTTGTTTCCATCCTCATTTTGAAAATGACAGAAACCCAAGC
TGGAGAATACCTACTTTTTATTCAGAGTGAAGCTACCAATTACACAATATTGTTTACAGTGAGTATAAGAAATACCCTGCTTTACACATTAAGA
AGACCTTACTTTAGAAAAATGGAAAACCAGGACGCCCTGGTCTGCATATCTGAGAGCGTTCCAGAGCCGATCGTGGAATGGGTGCTTTGCGATT
CACAGGGGGAAAGCTGTAAAGAAGAAAGTCCAGCTGTTGTTAAAAAGGAGGAAAAAGTGCTTCATGAATTATTTGGGACGGACATAAGGTGCTG
TGCCAGAAATGAACTGGGCAGGGAATGCACCAGGCTGTTCACAATAGATCTAAATCAAACTCCTCAGACCACATTCCCACAATTATTTCTTAAA
GTAGGGGAACCCTTATGGATAAGGTGCAAAGCTGTTCATGTGAACCATGGATTCGGGCTCACCTGGGAATTAGAAAACAAAGCACTCGAGGAGG
GCAACTACTTTGAGATGAGTACCTATTCAACAAACAGAACTATGATACGGATTCTGTTTGCTTTTGTATCATCAGTGGCAAGAAACGACACCGG
ATACTACACTTGTTCCTCTTCAAAGCATCCCAGTCAATCACCTTTCGTTACCATCGTAGGAAAGGGATTTATAAATGCTACCAATTCAAGTGAA
GATTATGAAATTGACCAATATGAAGAGTTTTGTTTTTCTGTCAGGTTTAAAGCCTACCCACAAATCAGATGTACGTGGACCTTCTCTCGAAAAT
CATTTCCTTGTGAGCAAAAGGGTCTTGATAACGGATACAGCATATCCAAGTTTTGCAATCATAAGCACCAGCCAGGAGAATATATATTCCATGC
AGAAAATGATGATGCCCAATTTACCAAAATGTTCACGCTGAATATAAGAAGGAAACCTCAAGTGCTCGCAGAAGCATCGGCAAGTCAGGCGTCC
TGTTTCTCGGATGGATACCCATTACCATCTTGGACCTGGAAGAAGTGTTCAGACAAGTCTCCCAACTGCACAGAAGAGATCAGAGAAGGAGTCT
GGAATAGAAAGGCTAACAGAAAAGTGTTTGGACAGTGGGTGTCGAGCAGTACTCTAAACATGAGTGAAGCCATAAAAGGGTTCCTGGTCAAGTG
CTGTGCATACAATTCCCTTCGCACATCTTGTGAGACGATCCTTTTAAACTCTCCAGCCCCCTTCCCTTTCATCCAAGACAAGACAACATTCTAT
GCAACAATTCGTGTTTCTCTCCTCTTCATTGTCGTTTTAACCCTGCTAATTTGTCACAAGTACAAAAAGCAATTTAGGTATGAAAGCCAGCTAC
AGATGGTACAGGTGACCGGCTCCTCAGATAATGAGTACTTCTACGTTGATTTCAGAGAATATGAATATGATCTCAAATGGGAGTTTCCAAGAGA
AAATTTAGAGTTTGGGAAGGTACTAGGATCAGGTGCTTTTGGAAAAGTGATGAACGCAACAGCTTATGGAATTAGCAAAACAGGAGTCTCAATC
CAGGTTGCCGTCAAAATGCTGAAAGAAAAAGCAGACAGCTCTGAAAGAGAGGCACTCATGTCAGAACTCAAGATGATGACCCAGCTGGGAAGCC
ACGAGAATATTGTGAACCTGCTGGGGGCGTGCACACTGTCAGGACCAATTTACTTGATTTTTGAATACTGTTGCTATGGTGATCTTCTCAACTA
TCTAAGAAGTAAAAGAGAAAAATTTCACAGGACTTGGACAGAGATTTTCAAGGAACACAATTTCAGTTTTTACCCCACTTTCCAATCACATCCA
AATTCCAGCATGCCTGGTTCAAGAGAAGTTCAGATACACCCGGACTCGGATCAAATCTCAGGGCTTCATGGGAATTCATTTCACTCTGAAGATG
AAATTGAATATGAAAACCAAAAAAGGCTGGAAGAAGAGGAGGACTTGAATGTGCTTACATTTGAAGATCTTCTTTGCTTTGCATATCAAGTTGC
CAAAGGAATGGAATTTCTGGAATTTAAGTCGTGTGTTCACAGAGACCTGGCCGCCAGGAACGTGCTTGTCACCCACGGGAAAGTGGTGAAGATA
TGTGACTTTGGATTGGCTCGAGATATCATGAGTGATTCCAACTATGTTGTCAGGGGCAATGCCCGTCTGCCTGTAAAATGGATGGCCCCCGAAA
GCCTGTTTGAAGGCATCTACACCATTAAGAGTGATGTCTGGTCATATGGAATATTACTGTGGGAAATCTTCTCACTTGGTGTGAATCCTTACCC
TGGCATTCCGGTTGATGCTAACTTCTACAAACTGATTCAAAATGGATTTAAAATGGATCAGCCATTTTATGCTACAGAAGAAATATACATTATA
ATGCAATCCTGCTGGGCTTTTGACTCAAGGAAACGGCCATCCTTCCCTAATTTGACTTCGTTTTTAGGATGTCAGCTGGCAGATGCAGAAGAAG
CGATGTATCAGAATGTGGATCGCCGTGTTTCGGAATGTCCTCACACCTACCAAAACAGGCGACCTTTCAGCAGAGAGATGGATTTGGGGCTACT
CTCTCCGCAGGCTCACGTCGAAGATTCGTAGAGGAACAATTTAGTTTTAAGGACTTCATCCCTCCACCTATCCCTAACAGGCTGTAGATTACCA
AAACAAGATTAATTTCATCACTAAAAGAAAATCTATTATCAACTGCTCCTTCACCAGACTTTTCTCTAGAAGCCGTCTGCGTTTACTCTTGTTT
TCAAAGCGACTTTTGTAAAATCAAATCATCCTGTCACAAGGCAGGAGGAGCTGATAATGAACTTTATTGGAGCATTGATCTGCATCCAAGGCCT
TCTCAGGCCGGCTTGAGTGAATTGTGTACCTGAAGTACAGTATATTCTTGTAAATACATAAAACAAAAGCATTTTGCTAAGGAGAAGCTAATAT
GATTTTTTAAGTCTATGTTTTAAAATAATATGTAAATTTTTCAGCTATTTAGTGATATATTTTATGGGTGGGAATAAAATTTCTACTACAG
HUMAN SEQUENCE - CODING
ATGCCGGCGTTGGCGCGCGACGCGGGCACCGTGCCGCTGCTCGTTGTTTTTTCTGCAATGATATTTGGGACTATTACAAATCAAGATCTGCCTG
TGATCAAGTGTGTTTTAATCAATCATAAGAACAATGATTCATCAGTGGGGAAGTCATCATCATATCCCATGGTATCAGAATCCCCGGAAGACCT
CGGGTGTGCGTTGAGACCCCAGAGCTCAGGGACAGTGTACGAAGCTGCCGCTGTGGAAGTGGATGTATCTGCTTCCATCACACTGCAAGTGCTG
GTCGATGCCCCAGGGAACATTTCCTGTCTCTGGGTCTTTAAGCACAGCTCCCTGAATTGCCAGCCACATTTTGATTTACAAAACACAGGAGTTG
TTTCCATGGTCATTTTGAAAATGACAGAAACCCAAGCTGGAGAATACCTACTTTTTATTCAGAGTGAAGCTACCAATTACACAATATTGTTTAC
AGTGAGTATAAGAAATACCCTGCTTTACACATTAAGAAGACCTTACTTTAGAAAAATGGAAAACCAGGACGCCCTGGTCTGCATATCTGAGAGC
GTTCCAGAGCCGATCGTGGAATGGGTGCTTTGCGATTCACAGGGGGAAAGCTGTAAAGAAGAAAGTCCAGCTGTTGTTAAAAAGGAGGAAAAAG
TGCTTCATGAATTATTTGGGACGGACATAAGGTGCTGTCCCAGAAATGAACTGGGCAGGGAATGCACCAGGCTGTTCACAATAGATCTAAATCA
AACTCCTCAGACCACATTGCCACAATTATTTCTTAAAGTAGGGGAACCCTTATGGATAAGGTGCAAAGCTGTTCATGTGAACCATGGATTCGGG
CTCACCTGGGAATTAGAAAACAAAGCACTCGAGGACGGCAACTACTTTGAGATCAGTACCTATTCAACAAACAGAACTATGATACGGATTCTGT
TTGCTTTTGTATCATCAGTGGCAAGAAACGACACCGGATACTACACTTGTTCCTCTTCAAAGCATCCCAGTCAATCAGCTTTGGTTACCATCGT
AGGAAAGGGATTTATAAATGCTACCAATTCAAGTGAAGATTATGAAATTGACCAATATGAAGAGTTTTGTTTTTCTGTCAGGTTTAAAGCCTAC
CCACAAATCAGATGTACGTCGACCTTCTCTCGAAAATCATTTCCTTGTGAGCAAAAGGGTCTTGATAACGGATACAGCATATCCAAGTTTTGCA
ATCATAAGCACCAGCCAGGAGAATATATATTCCATGCAGAAAATGATGATGCCCAATTTACCAAAATGTTCACGCTGAATATAAGAAGGAAACC
TCAAGTGCTCGCAGAAGCATCGGCAAGTCAGGCGTCCTGTTTCTCGGATGGATACCCATTACCATCTTGGACCTGGAAGAAGTGTTCAGACAAG
TCTCCCAACTGCACAGAAGAGATCACAGAAGGAGTCTGGAATAGAAAGGCTAACAGAAAAGTGTTTGGACAGTGGGTGTCGAGCAGTACTCTAA
ACATGAGTGAAGCCATAAAAGGGTTCCTGGTCAAGTGCTGTGCATACAATTCCCTTGGCACATCTTGTGAGACGATCCTTTTAAACTCTCCAGG
CCCCTTCCCTTTCATCCAAGACAACATCTCATTCTATGCAACAATTGGTGTTTGTCTCCTCTTCATTGTCGTTTTAACCCTGCTAATTTGTCAC
AAGTACAAAAAGCAATTTAGGTATGAAAGCCAGCTACAGATGGTACAGGTGACCGGCTCCTCAGATAATGAGTACTTCTACGTTGATTTCAGAG
AATATGAATATGATCTCAAATGGGACTTTCCAAGAGAAAATTTAGAGTTTGGGAAGGTACTAGGATCAGGTGCTTTTGGAAAAGTGATGAACGC
AACAGCTTATGGAATTAGCAAAACAGGAGTCTCAATCCAGGTTGCCGTCAAAATGCTGAAAGAAAAAGCAGACAGCTCTGAAAGAGAGGCACTC
ATGTCAGAACTCAAGATGATGACCCAGCTGGGAAGCCACGAGAATATTGTGAACCTGCTGGGGGCGTGCACACTGTCAGGACCAATTTACTTGA
TTTTTGAATACTGTTGCTATGGTGATCTTCTCAACTATCTAAGAAGTAAAAGAGAAAAATTTCACAGGACTTGCACAGAGATTTTCAAGGAACA
CAATTTCAGTTTTTACCCCACTTTCCAATCACATCCAAATTCCAGCATGCCTGGTTCAAGAGAAGTTCAGATACACCCGGACTCGGATCAAATC
TCAGGGCTTCATGGGAATTCATTTCACTCTGAAGATGAAATTGAATATGAAAACCAAAAAAGGCTGGAAGAAGAGGAGGACTTGAATGTGCTTA
CATTTGAAGATCTTCTTTGCTTTGCATATCAAGTTGCCAAAGGAATGGAATTTCTGGAATTTAAGTCGTGTGTTCACAGAGACCTGGCCGCCAG
GAACGTGCTTGTCACCCACGGGAAAGTGGTGAAGATATGTGACTTTGGATTGGCTCGAGATATCATGAGTGATTCCAACTATGTTGTCAGGGGC
AATGCCCGTCTGCCTGTAAAATGGATGGCCCCCGAAAGCCTGTTTGAAGGCATCTACACCATTAAGAGTGATGTCTGGTCATATGGAATATTAC
TGTGGGAAATCTTCTCACTTGGTGTGAATCCTTACCCTGGCATTCCGGTTGATGCTAACTTCTACAAACTGATTCAAAATGGATTTAAAATGGA
TCAGCCATTTTATGCTACAGAAGAAATATACATTATAATGCAATCCTGCTGGGCTTTTGACTCAAGGAAACGGCCATCCTTCCCTAATTTGACT
TCGTTTTTAGGATGTCAGCTGGCAGATGCAGAAGAGCGATGTATCAGAATGTGGATGGCCGTGTTTCCGGAATGTCCTCACACCTACCAAAACA
GGCGACCTTTCAGCAGAGAGATGGATTTGGGGCTACTCTCTCCGAGGCTCAGGTCGAAGATTCGTAG

TABLE 24

MOUSE NOMENCLATURE
ICSGNM Dntt
Celera mCG2378
HUMAN NOMENCLATURE
HGNC DNTT
Celera hCG24648
MOUSE SEQUENCE - GENOMIC
GATGCTCAGCCCTCCAGGGCAGGAGAGGGCTATACTCTAAGTGGAGAGATAAGCTACACTTGTCCATTTATAGAGTGGTAGGTAGGTATATTAA
CGGTGTGTTCTCTGAGGAAGTCTCCAGGACCTGAGATGAGGGATGTGTGTGGGGTCAGCATCTCTGGGGCAGTGGGAGAAGACTGGTCAGGAGG
AATCACGGTAGAGTATATCAACACAGGCAGTTGTTAGAGCTCTGTGATCATTCAGTGCTATCACCCATGGCAACAACCTCTCCCAACCATTGAT
TGGGAATTGGTGTGGGTTTCCCCTAAGGGAAGTCCTGACAGAGATGTGGAGGCATTTTCTCTGAGCCATCAGCCTCCTGTCTGAGGACCTGGGG
AGGCTTTGTCAGCATCCTCTATAGAAGGTCAGTGTCTTGGTGATCACTGTCTATGCTGGAGAGAAGAGAGAGAGGGTCTGAGGCTAAGGGGCTG
GACGTGATCAGGGGATCTTGTAGTGATATCAGTTTCTGTTTACTAGCCGTTTTACCCCCACTTAAGACAGCGTAAACTCCCAGAGCTACAGTTA
TTCCCATTGTCTTAGGTCCCCCGATAAAACTCAGGAAAGAAAACCAAAACCATTTAAAAGATCTCTTCAAGCCACGCAGGTCAGACGCTCACCA
AGTTTAGTCTAACTCGACTAACATTTTTGTTGTTGTTGTTTTGTTTTGTTTTGTTTTTTTGAGACAGGGTTTCTCTGTGCATCCCTGGCTGTCC
TGGAACTCACTCTGTAGACCAGGTTGGTCTCGAACTCGAAATCTGCCTGCCTCTACCTCCTAAGTGCTGGGATCAAAGGCGTGTGCCACCACTT
TCCTGGCTTCAACACTTTTTTTCTTTCTTTCTTTTTTTTTTTTACTGTTTTTGTTAACAGTTCTATTCGGTCTTTCAATTAGTTGTCTAAAGAC
TGCAAAAGTCACTTTTGAAATTGCTTCTTTCTGACTTGACTCCTTAGCCCAGCAAGTTTGTCCTCTAATCCCCACTCTTATGGTTTGCATCTCA
GACCCCTGCTTAAATTTTACCTTCTACTTAATTGCACTGAAAACAAGTAAAGCCTTCTCAGAGCAACTTGGTTAAAATCGGGTCCAGTCCCACC
CACAGACACCTGCCCCCTTCCTCCTTCCCACAGCATGTACACCAGCCACCTCTAGTGGATTATTTACCGTCTGTTTTTTATCTAATAAATGGTA
AGCTCTGAGAGGGCAGGGACCAGCTGTTTGAGCCTGGAAGAACTGGCTTCTGGAGGGCACCCGACAGAACTGTAGGTTGAACTCATGGCTGGAC
TCTCTGCTTCCATTAGCTCCACTCTGGCCATTGGCAACAATGTCTAGCTGGAATCACAGCTCTGAGTCTCCATCCTAATCCTCTGGGTAGATCC
TGTTAAGCCAGCATGCTCACCTCCAGCGATAGCTGAAGGTCAGGACAAATGTTAAAGTGTTTCCTAATACAGACCATCTCTATAGATGTCCACG
ATTACTAATATCTCAGCGTATTGTTGACTTCTCAGTGAAAGGGTATGTGCTCTGAAAGCTGTGACCAGAACTGAGTGAGATTTTCCAGGTTATG
GGTAGTGGCACAGGCTGGTCGACTCCATGTGTAGAAGCCACCTGAGGACAGCCACCTGAAGAGCGTGAGTGGCTGAGAGTTCAAGAGCTTGTAA
TGTTCACCTGCCATATCTACTGCTCTCTGACCCGGTACTCACGGCTTATCAGCAATCCAGAAGGAGAGGGTATTTAGCGGGTTGCCTCAAGAGT
TTACTGAGCAAAAGAGAGGCTGGAAAAGATTTCAGAGATAGTGGGGAGTGGTGGTGAATAACCCTGTCTTAGGATTTTGGAAATGTTACTAAGT
GGAATTAAATAGTCCCGGAAGTGAAGACAGAGATGCAGACCAGGCATTGGGGCTCCCACTTGTAACTTCCACACTCAAGAGATAGAGGCAGAAA
GACAGAGCGTTCAAAGTCATCCTGAGATCAAGGCAGTGCAGCCTGGCTACAGGAGACCCTACTTCAAGAAGAAAAGCAAAGAGCAAGAAGGGGA
ATGGGCTAGAGATACAAATGCAATAGAAGCCAGGAATGGCAGGGCTGGAGGGCTCTGCAGGCTACCAGGGGCCATGGATGAGAAGCCTTTCTTC
CAGCCAGCGACCAACAAGCAGTTCAGCCTGTGGTCTGGGGCTGCCATCCCACTTGAGACTCTGTTCCCATTCCCGTCTGGTCATAGTTCACATC
TCACAACTATCATCTTCTCATTGGTTCCCTCTCTCTTCTCTGATTCACCCTTGCCTAGTTGGTTTTCAGGAGGTGCCATATTGCTCAGTGGGTC
TCCATGCAAACTCATATCAACGGCTCCCACGCCTGCAAACAAAACCCCAGCTCTCCTATCTACACCTTTCCTGTGGGCAACTGCCTGTCCCTCC
TCACACCCACAGGGGATGCTCAGGATGTAGCCTGCTCATTTTGTGTTCCTCTGGAGTTTAATAAATATGGTTGGATGAAAACTAGAGGCCACCA
GTACCATGATATGTCCATCGACTCCCTTGTCCTGTCCTGTCTCACTTGGTAGATAACAAAATAAAGTGGAAATACAGAATGTTCTTGTATTAGG
CACTCAGATGATAACTATTACTGTGGGTGGCTTCTCAATGACAAGAAACTTTGAGAAACTCTATAGTTTTCCCCAGTGGGGCAGTTCCAGAAGA
CACCTGCTGTGAAGAGCCAAGAACAGCTGTGCAGGCAATAGAGGAAGCTGGCACTCACTCAGTAAAAAAACCAAACCAAAACAAGACCCTGCCT
GCAGTTGGATGGCAAAGTTCCCAGATGGGCAGCTTGACATAAAGAGGCGCTGTGAGGTGTTTGATATAAAAGAAGATAAAACCGCCGGAAACAG
ACATCTCCTCTCCTAAACTGACTCCCTCAATGTGTGCAAAGAACAACCCGGCTCAGGGTAAAAATAGTGTTTGTGGATGAAGGGGGATGTGTTT
GTCCACCCAGGGAAAGACTGCAGCTGGCCAGCCTCTTTCCTGCACAGTGGAGGGTTTGAAGGACAGTCAGATGGGTGACCAAGGAGGAGAGAGC
AGGGTGGAGATACAGATGTGGATCCATATTAAACCCAGAGGAGAAGCAACCGCTCAGGAATGGCAGAATGGTGGCCAGATGTGGCCTTTGTCTC
CTCATCCCTGTGACATCATATCCAGGCAGGAGCACCAGTCATGCCAGCTAGCACCTTGGAGTGGCAGGGTGGGAGGGCAGGGAGAATGGTGAGT
GGTGCCCAAGCAGGCCAGGCAGGCAGAGGAAGTTTTGTCTTAGGAGGCCGTGAAGGTCAGTACTCTGTTGGAAGCTGAGGAGTCCACCGGTGAC
AGAAATTGCCCAGGCAGACACACCCAAAAAGTGTCAAGTGGCTCAGGCTTGTCTAGTCCGGGGTCATGATACTCCTTACTAAGTCAATAGATTG
TCTAAGTACTGTGCAAAACAGCAAACAGAACAGGGCCGAGCCGGGCTCCAGACTCAACAAACCAAGCAGGATTCTTTATTGCAGGGCCCCATGA
GGTGTTGGACATTTTAATACTCGTTGCATAGAGTTTGTCTGATCCCTCTGACCTAACTCATTACAGAATCATCATCATCATCAACTTTATCAGC
AGTCATCAACAGAGTCTCACTGTATAATCCAGGCTGGCCTCAAACTCATGACCCTGCTGCCTCAATTTCCCCGTTTCTGGGATCTCAAGTGTGC
TCCACCATACCCAGCACTGAAGACTGTCTTGTTTGGCTTGGAGCATCTCCCTGAGACCCTGGGCTCCTTGCAGATCTCTTCACCAGATCTCTTT
ACCCTCCCTAGGATGCAGAGTCTACCTTGAGTGGCACTGGCAGAAATATTGGTGAAGTGCAAAGAGACACAGGGACGTGAAGGGGGAACAAAGA
GAAGTCACAGCCTTTCAGTTAACAGTATTGTCCTGAGAAAGCACTGCATGCCAATATTCCTGTATTTAGTCTTCATAAACACTCACAAGACAGA
AACCACTAGAACCTCACTGTGCATATTAGGGAACTGAGGCACAGGAATGTCTGGCAACACACCTGTGGGTATGTCTGTGTGTAGCTCATAATGC
TGTGTGTTATGGGCAAGACAGAAAGATGGTCGGGGCAGGAAGGGCCACCAGAAAAACCTGGGAAAACCCTCCATCACTTTGTTCACAGGGCTCC
CACCAGATCAAGTTTGGAAGTATTAGTTGATGTGGTTCAGGGAGACCAGACCTCCTTGTGTGTTCACATCAACAACTGAGAACTCTAGCCTGAG
CACAGCAGTGGGTGTTGCTTCCACTCAGGTGGGGAGAGGGTGGTCCTGGGGCCTCCCTATTCCCTTTCCTGGCATCACGCACATTCCCCATCAT
CCCCTTGACATCAGCCAGCATATAATCCTGACATGTCAGGAACATGTATTCTCCCACATCCCACATGCTCTCCTGTGGCCCTGCCATAGAGAGG
TAGACTCTTGAGGCTACTCTAACCAATAGGCAATGAACAACAGCAATGTGTATTACTTGGCATTGAGTGTGGGTACTAGGTAATCCTGCTGTGA
TCTCTGTCCGACTGCAGCAGTGGCTGAGACGGTCCAGATGTGGCAGGTACAAACATGTCAAGCTTCCATTTGGCCTGGATTTAGACTAGAGCAG
AACCCTCCAACAAGTAGCATGAAGGAAAAAAATAGACTTTGATTTGTTTAGCCAACACAAAGGATTTGGAACTAATTGGCCATTGACTGGACCA
AGGAGCCACTTAACACCTGATGCAATTAGCACCAGAAAATTATGGTATAGAAGGAAGAAATTAACTCCTGCCGCTATCAGGAATGCCAATGGAA
ATAACAAAACAATTGACTTTTGTATAGTGTTTCATAGTGTGTGTGTGTGTGTGTGTGTGTGTGTGTGTGTGTGTGTGTATTCAGAGGACAACCT
AGGATGCCACCCACTTGTTTTGAGACAATGTCTCTCACTCTAATTAGGCGAGGCTGGCTGGGCAATAAACCCTAGTAATCTGCCTGTCCCTGTC
ACCCTAGCACTGGATTACAAGTTCATGCTGTCACAACCAGCTTTTAATACGAATACCATGGGATCAAACTCACATCCTTCATGCTTGGGTGGCT
AACACTTTACCAACTGAGATAGCTCCCCAGACCCATGTTTCACAATGTTCAAGGTACTTGATAAACAGAGTTTAATTAATTCCAAAAGTGACCA
CCATGCTTCCATGATTCTGAGCAGTTTTCCACCTCCTTTCATTAAGCCGTGACTGTGTAGTCCATTTTCAGCTGTTAGAGCAAACCATCTGAAA
CTGGGTAATTTATAAGACTTATTTTGGCTAACCGTTCTGCAGGCTGGGATGTCACTGAACATAGATAAGGTTGCATTCTTCTTCCCATGGTGGG
AACAGAAAGGCAAGAGGGGTGTGTGTGGCAGGGATGAATAATGAAGGGTGGCTTTATGTTTCAACAACCCTGCATCCAACCTGTGACAAAGGCA
CTAATCCTTTTTGATGTCTAGTTGCCCCTCCAGGTCCACCTGGGAACAATGCTCCAATGGCAATAAAGTTCCCATATAGGTTTCGAGGGTAAAA
ACTATTTTCAAATCATAGTACAAAGGCTTCAGGCTACATGATGCTATTGTGAAGGACCCCGAGAGTCAGAAAACTGGGATTGGAAGCCCCAGAC
TTCATCTCCAGAGTTGCTTAGGGTGAGATTCCTAAACGGTTCTAACTCCAACCCCTCCTTTCCCCACCATCAGGGCTCTGGATCAGTAGAGTAT
TGCCTGACAGTCAGGCAGGACCTCACACAGGATGAAGCAGACACATGTCCACCCTTTATTCAGCTCACTTAAAATAATCTCACCACCTTTGAGT
CACCAAGATGTAAACACAAGAGAAAAAAATTAAAAGTAATTTACTGTTGTGGCTAAAATACAATTCCTGTCTATTGTAGGAGTCTGTTAAACAA
CAGCCAAACATAGTGTGTAGTACAAGTTGCTCCAAGAGGTGGCGTTGGGAAAGGCCTTGTAAGTTGCTGGGTTAGATCAGCCTGGGAAGCATGC
TTGACTCCATCTCAAAGAAAAATAAATAAGGAAAGGAGAGGAAGGAAAGAAATAAGTCTGTAAAAGAAAAATGCACAAAGACAAAGCTAAAAGT
CCTGCCTAAACCCCCTGAGTGGCAGGATTCTCTGTTCTGATGTAGGGCTGGTCATATGCCTCCTTGCTCCTCTCTATGAGAATTGGTCACCACG
TATTATGAGCTGGTTTTGTAATTATTAGATTGTGCACATTTACACATGCCATTAACTAGTTTATGGCATGACTTACCTGTTACCCTTTATCCAA
CAGCTGGCTAGTAGACAGCTCGTGTTGTTTCTAAACTCTATGTACAGATCAGGCCATGATGAGCTGCCTGGTATGTGCGTCTTCCTGAGTCTCT
CTTCAGATGGAGTATGCCTCTAGTGGTGTGGCTAATGCCCTGCGTGTACCTCTGCTCTCATTGTATTAAAGATTCAGTTGGGTGTTTCATACAA
GAGAGAATTTACCATAGAAGACTTCTTTTTTATTTGTTTGGTTTTTTTGTTTGTTTGTTTTTTTGACACAGGGTTTCTCTGTGTAGCCCTGGCT
GTCCTGGAACTCACTTTGTAGACCAGGCTGGCCTTGAACTTGGAAATCTGCCTGCCTCTGCCTCCCGAGTGCTGGGATTAAAGGCGTGCGCCAC
CACACCCGGCCATAGAAGACTTCTTTAATGGGCACTGGAAATCTAGGAATCAGAAAAGGACACTGAGTCAGCTCAGACTGACTGTAGAAAACAC
GTCCCACTCTTGGGTGCAGCAGTAGTTAACACTGGTCAATGTGATACTTGGATAACTTAACAATTTTATAAACTATAGATCTGATTTTAGATTG
AGCTAAGTTGACCCTCTGAACAAAATAAAAGTTACTATTGACTCTTACTTAAAGTTCTCCTCTGTGTCACGCCTTCTGTACGGGGGTGAAATGG
GCTATCTCAGTTCTTGATTTAGATGTGGGGAAAACAGGCTTAAGGAAGTGAAGCAACTACGCAAGGTCACAGCCTCCCAGCTGTGCAGATGGGA
GCTCGTGCATCCCTGGCTGCATCCTCTGTCTGTGTGCTATTCTCTGTTCCTATTGCTGCTTCTGTGGTCTCACATTGCTGTCCAGATAGAAGCT
TGTGCATCTATGGCGGCATCCTCTGTCTGTGTGCTATTCTCTGTCCCCGTTGCTGCTTCTGTGGACTCACGTTACTATGGCGATGATACCACCC
CGGGGCCTAGCTAGAGGACTGCCTTGTTCATGCTGTCAGACCACTTGGAGGCTAAGACTAATTCTATCATGTAGCTGTCTGAAACCAAGCATCT
AACAGCGGGCCAAACTGTGTGTGGGGGGAGGCTTCTCCCCACTCATTGGTCTCTATCAAGCAAACATTGCACCCTAGTCTCACAGAGCTGCACA
AAAGGCCCCTCCAGATGATTAAGTTCCTTCTGCTACATGTCCGTGAACACCGTCAAGGTCTCTACCACACAGGGAGCCCACTCTGCACTTTTGA
CAGGTGTACATACTTGCACAGAGCCAAAGTCTGTTCACTACAGCTCTCTGTCATTCTTGCATGAAACAGCCTTGTCGATGCCCGATGGCAGCTG
TCATAGCACCCTCCTTTGGCAGTGCTGAGGTGCTCATGCGTTCATACGCTCTTGAAGGCAGTAAAAGTCCCAAATGCTGTGGTGGAATACATGA
GAAGGGTCCATATTTATTACTGGGTAAGGAGGCATGACAGAAGGAGGCTGTTTGGTATGTGTATGTGTGTAAATACATGTGTGTGTGTGTACAC
ATATATATTTGTTTTTGCTTTTTGTTTTTTGTTTTTTTTTTGTTTTTTTTTTTTTTTTGTTTTGTTTTTTGAGGCAGGCTTTCTCTGTGTAGCC
CTGGCTGCCCTGGAACTGACTCTATAGACCAGGCTGACCTTGAACTCACAGAGATCTTCCTACCTCTGCCTCCTAAGTGCTGGGATTAAAGGCA
TTGTACTACCAGTCTGGTTAAGAGCATACATTTTTAATTTTGATTTTTTAGTTTATGTNNNNNNNNNNNNNNNNNNNNCTCCTTGTGGGTGAGC
CTCACCACCCATTCAATGTTCTCTCACACAGGCCCTCACACAGAAGCACCAGCTCTTAGGAGTAACTGAAAGTTTCCTGCTAGTAACCTTTGTT
AGACCCAGTTCTACCCACAATAGTTTACATAAAAATGAAGCAACAAAGGAACCCCCGGGAAGACCGTTGCTTTGAAGCACAAATGTAAACGGTC
TGGTTTCAGCACCCTCAGATTGCTTTCTGTGCAGAAAAGGGCTAAACAATCACTGCCTGCCTCACTGGCAAGGTCACATTCCTGGAATTACTCC
TTACACTGTAGGAGACGCCAGGCACAACCGCTGTTATTTGGGAGAATGTTCTTGTAAACCCTTTCTTCCTCTCTGACTGCACAGCCTTCTCTCA
AGCTCATGACCACAGCTGTGTGGGCATCTCCAGATGACACGAGGGAGCTGGTCCTCCCATAGCACTTATCTGTGATCCACTGTGGTTAACTGAC
ATTTTGTAGAGTTCTTTCCCTCTCCCTGTAGGTGAACCATCAGCCCCCCCCCCCCCGTATTATATGGGTATCTAGCTGGCAGTATGGTCTTATA
AGAATTCAGTCAGCAAAGGTTTTGAGGTCTTGCCTAGGTCCTGTAGTAGGTGTTCCAGAAGTGAGACCTACTAAGTAATATTAACTAAAGATGA
AATTTTCATTTTGCACAGGGCTGTGAAAATGTCCCTAACTCCATGCCCATGACTCTTCTCCAAAACTCATCTCGTCTCAGAGACCTATGTTAGT
TATGTAGCGACCACTCTCTGGAAACTTAGTGCACTGAAAAAATAGAAACCAAAAACAAACTAAGTTGCATCACGGTTGAAGTCTGAAGAACTGC
ATTCATGATCTCATTTGCCCCATACTCTGTGATCTGCAGTGTCTGAACCTTAGCTTTCTATAAAATGTGCTCACACACTGGAGCCTGTCGTCCT
GTGCCCACTGCAAAACGATAGTAACTAAAGCAGCAGAGTAGACAGAGCCAGGAAAATCACAGTTATGGGCTTGTTGGGAAAAAAGTGACCATGG
GGCCAGGAGAGATAGCTCAGTGATTAGGAGCACTAACTGCTCTTCCAGCAGGCCAGAGTTCAAGTCTCAGCAACCATGTGGCAGCTTACAACTG
TCTGCAATTCCAGTTCCAGGCAATCTGACACCTTCCAACACCAATGCACATAAAATAAAAAAAAATTAAAAAAAAAAAAGCAACCAGCAGAGTG
AGAAATTCAGGTGGCATTCACGTGAAATCACAACACCTGTATGTAACACATCTGTGCCAATTACAGACATCCAGTGTACCATAACGTAGGTCCG
AGAGAAAGAATAGAAGTGAGCTAGAGTAAAGCAGGAATCAATGAATAAAGTCAGCAGGAGCAGTTAGATGACTAAGTGGTGATGATAGAAGCTT
GCTACCAAGACTGACAACCCACGTTTGATTCCTGGCACCTACAACGGTGGAGGGGGAAAGATGCCTCTGACTGCCACACTCATGTCATGGCATG
CACACACATGCCCCCTTGCGCAGTAAGCAAATGTAATACATTTTTTAAAAAATGGCTTTTAAAATGCAACCTAAAGGGTTTCAACATGTCTAAA
GATAAATAACAGTACTCAACCAGTGGCGAGGTCCCCCTTTCTTCATAAGTTTATTCTGACGTACTGCCGAGATCAAGTGTATGAAAACCCAAGT
TCACTGTTCTTTCTGTCAAGAGCTCCATTTTGGGAGAAAGGACCTGATCAGAGTGAGACATTCCTTCAGCAGGAAGTTGTCACCAGGGTGGTAC
CTATGGGTCTGCTGGTGAGAGGACATCAGAGCCCTCATTCTGGAGACACCACCTGATGGCACAGACAGAGCTAGACTGTCTGCTTCCATGGATC
CACTGCAACCAGTCCACCTGGGTCCTCGGAACAAAAGACCCAGCCAGCTGGGCACCCCAGTGGCTTCCACGCCTTATGACATCCGATTTCGAGA
CTTGGTCCTCTTCATTTTGGAAAAGAAGATGGGAACAACTCGAAGACCCTTCCTCATGGAGCTGGCCCGAAGGAAAGGGTTCAGGGTGGAAAAT
GAGCTCAGGTAGGACAGCCCTGGTCACTTTGCAAGCATGCAAGAGCCCTGAGGACATCCCCAAGGGACCCAAGGGGCCAGTTGGCTAAGGGAGA
TGAGTGGGGACTAAATGAGATTATATAGAAACTAATTGGTACCAAAAAATAATATTTTTTAAAAAAAATATGGCTTTCCATGACTGACTCTGAG
ATTAAGTATCCAGGAGTGATAACCAGAAAACTAGTCAGAGAACCACATAGAAACCACTCGATTCCATTCTGTACCAAACTGAACCAACACCATT
GGGTTCAATTTTTTACATTGCTACTTTGGAGCAGGGAGGGTGTTCTGATTGTCTGGCTCTGACTTATACACACCTGCTCTGCTAAGGGTCTATG
GTGGGCTAGGCTGAGGTGAGTCACCACGGGTACCTTAATGGCCTTGGTGATGGTTTTCCAAAGGGTGGTCCAAGTGGGCTTCAGGAAGAGATGC
TGGTTAAAGATGTTTGTCTAGGGGTGACAGATGGCACCCCAGTGTGCTATGATCAGAGTCTAGGCAGGGAGGTGATTGAAGCTACCATCTGCAC
TCGTTCAGTAAGCACCAATTATCATTGTTTTGCTGACTATTGTCTACGTTCACTCTCTTGACCCAATGAAAAGAATCAAAGCGATTCAGAAAGT
CCCAGTAAAAGGCCCTGGTCTGCCTGACGCCTGCAGACACAAGTGTTCCGCTTACAGTGGGAGTGGGGGGGGCACTCAGTTCACTGCCTTCAGC
CATGCAAGTTGGGAGGGGTGCTGAACATACCGTGTCAGAGATGATCTCACTTTTACACTTGGGATTTTGGAAAGTCTTTGAATTTAAAAGAAAA
TTGCCAAACGTTTCTTGCTGATGAAATGCTGGCTTTGGAGACATTGAGGAGACACATTTCCAAGGTCAGTACTCAGGAATTTCACACTCCACAT
GCCCAGCTGAGGGGATCTTTTTAGATTTTTTGCTAAAATGTCCCCAATCAAAAATTCCTGAGGTTCTGGGCTTCTCTCTTTTCTGAGTCTTTGT
CACATGGTCTCCTATTTTTTGTTTTAAGTCATCACAGCCACTCTCAACTTCATTCCTTGATGTTCTTTTCACACAATCCATTATTTCAACTCGT
TTTTGAGTATAACTAATCTGTTAGCCTTATGTACAGTGTTAGCTTTAAAAGAAAAAGAGATTAGATAGGTAAAGGGGGAGGGATAAAACAGAAA
AAACATCTTCAAAGAGAAAGTTAAAAACAGAAATCAAAAGTTTTAGCAGCTACCAAGATGCTTTTCATGGTAAAGGAACTGAACAGGGACCAGT
CAGGAAAGGGAGAGCCATCAGGCACGGTGCCACTCACTCAGAAAGCTCTCTGATAAAAGGTCCATACATCTGTTGGAGGCCCATGGACTGAGTC
ACCAATGCAGCAAATGAGCCGAGGCAAGTGTCAGGGGTGATGATGATCACGAAGCAAGTATCTTTGCTGGAGAGGTGAATCAGAGTGGTGCCCC
AGATGCCAGCAGCCAGAGCTATGCCTGCTGCTGGGGAGACAGGAAATGGGTGAGCAAGCCGCTGCTGTGCTCAGGATGGAGCAAAGTTCTCAGG
CGGAGAGCACAGACACTTTCAACGTGTGTGTGTGTGTGTGTGTGTGTGCGCGCGTGCTGTAGAGGAAGATGAATCACAGGAAGCAGAAGCCCCG
ACGATAACAGAATTTCTGTGCCCAGATAAAGAGTCATTAATTACTGTAGGGAGAACCTTTAGGCTTCATCTCCTGGTGCTGGCCCTTTCCAGAC
CAGCAGTTNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNATTTAACCC
TTGGAACCCCAGTAAAGGAGGAAGGAAACCAGCTCTACAGAGGTGATTTCTGACCTGCACATGTGCACCATGTCATATGCTAATGTGCATATAC
AATCATTGTAATTACTGCTAGTGATGATTAATTAAAAGGAAAAATGTCAATGGTACTGATTAACAGTAATGGCTACACTTCTTTCTTAACTTGT
AAAATTCAAAGGCAAAGAGAATGCTAAGCTCTGGAACAGGCTAATGTCCACATCTCCTGTATACTTTTGGCTGCCAAGAGGCTCAGTCTTGGCT
CCTACCTTCCCTGAGATCCTCTATGTACTGAGTTCTCTGGATTGAGTCTGTTCTCTCCAACATGAATGTGTCACTTTGAGTGGAATATTCAACT
ATTCTATGTGCCAATAGAAAGCTGGTTCCTAAAACATGAAAGAGAAGAGATGCTAAGTTCCTTGAGTAGTTCTGAACATCGAAGAACAGGATAT
TGAATTTATTCTGTCACCAGCATCCTAGACTTTAGAGCAGGTTGCTAACTTCAGCTCACCTAATACAGGCTCAACAAGGGAACCAAAATCTCCT
GTTTTCCATGCCCTGATTGCATCCTGAGTTCCCATAGAGTGAGATACTTGAAAGCTCTCATCTGTCATCTCCCCTTCTCTATAAATACTTCTGC
TACCTTCCCCTGTGTCTTGAAGCAAATCACTGTAGTCAACGCAGAAGAATATGTAGCTCGGAAGGATGGCGTTCCATCTTTCTAACCATCCAGG
CGCTTCATGCTAGCTTGCTAACAATGCTCAGGTTGTTGTAAACACATCAGAGGACAGCATTGTCCTGCAATGGAAGATGCTCTCTTAGCTGAAA
GCATCACAAAAGCCTTTGAGTGCCTGCTCTGTGCAGTATGTGTGGAGCTCTGGAAAAACAGAAGAAGACATGAGCCTTGCAAGCAAAGAGCTTG
CTATGATAAGGAAATCATGAATTGTATCTTGAATTGTGCTATTATCTTAATTACTTTTCTTTCTTTTAAAAAAATATTTACTTTTATCTTCGTG
TATCAATTAATGATCTGCCTGCAGGTACGTTTATATGTATATATGCATGTATGTATGTGCATATGTATGTATGTCTGTACATATGTATGTATGT
GTACCACATGGGTACAGCCCCTTCAGATAGCAGAAGAGCGTGTTGGATGGTCTGGAACCGGGTTTACAGATGGTTGTGAGTCATGTCACACACC
ATCTCTAGGAATCGAACCTCCATCCTCTGCAAGAGCAGGATGTTCTCTTAACCACGGAACCAACTCTCCAGCCCCTCGGATTCTCTTCTTCCTC
CAATAAAGTACACTGGCAAAAGCAAATTAAGGAAGACAGGGATTAGAGCTCACAGTTTACAGTCCATCATATCAAGGAAGTCACAGCAGCAGGA
GCTTGGGAGAGCTGGTCACCTCACACAGGTGACCAGGTACAGAGAGCAGTAAATGCCTCGCGTCCTGACATGTGACTGTAGACAGACTGTGATC
AAAAACTAGCTTTAGCCTCCTCAACAACTCATTCCTTTCGGACACCTTGTAACAGGTGTGTGTTGGCTAGTCTGGCTGCCTCTCCCCCCCACCC
CCCATCTGTTCACTGTTTGACCTGTTCACAGTGCGACTTTCACTGGAAAGTCATTATCTCAGAGCATTTGCCTTCAAAGCCCACGGGGGACCTT
GTGGTAATAAAACCCACATTTTCTATTTCAAAAATTATGAGCTTCAAGTTAATGCAAAGTTTTATAATACCATTAAAGGTTGTTAAAGAATCAG
GCAAGATCCTGACCAAGGCTGGAAGAGAAAGCTGTCTCTTTTGAATGCTGCTGTGTGTAGATTAGAACCAACCTCAGCTGCCTTGCTCTCAAGG
GTTCTAAAACACATTGGTTGCTCAGAGAACTGACAGAGCTGTGGTTGGGGTGGGTTGAGCCAGCTTCTTTAAGCAGCTCAGTTTAAGTATAAAT
ATGGCTAATGTCATTGTTTTCAAAATACCCAACATGACCTCTCCCAGGTGACAAGACCATCCATGAGAGAGATAACACTCACACTCGAAAGCGT
GGTGGGTAACGATGGCTGCCTCTCTTATTCATAGAAGGCATCTTTAATTATATGCAGTGAGGCACTGCCAGCCAGGAAGAGAAGGCATGAACAG
GCAGAGTGGTATATAAGAAAGATGAGAAAAGCCAGGAGTGGTGGTGCACACCTTTAATCCCAGCACTTGGGAGGCAGAGACAGGTGAACTTCTG
AGTTCGACGCCAGCCTGGTCTACAGAGTGAGTTCCAGGACAGCCAAGGCTATACAGAGAAACCCTGTCTCAAAAAAAAAAAATGAGAAAAATAC
TCAGAGCTAAGATTTGTGAGTTAATCCCATAATACATAGTTTCCGAACACCTGGAGCCCTACCTGACAATCCTAGCAATATTTTGAACATTACA
TGAAATTATACTTCTCATATATATTCTACTTAACATATAATAACTGCATATACCTAGGGGGGATAGAATAATAATTGAATATGCATATATAGCA
TGTAAGGATCAAAGTGACTCTGTCTCTTTATAGCTCTATCATTATAGCCACCTGTCGTAGTCAGGGTTTCTATTTTCTACAAACATCAGGACCA
AGAAGCAAGTTGGGGAGGAAAGGGTTTATTCAGCTTACCCTTCCACATTGCTGTTCATCACCAAAGAAAGTCAGGACTGGAACTCAAGCAGGTC
AGGAAGCAGGAGCTGATGCAGAGGCCATGGAGGGATCTTACTTACTGGCTTGCTCAGCTTCCTCTCTTATAGAACCCAAGACCACCAGGCCAGC
GATAGCACCACCCACAAGGATCCCTTCCCCTCATTGATCACTAATTGATAAAATGCCTTACAGCTGGATCTCATGGAGGCATTTCCTCAAGGAA
GGCTCCTTTCTCTGTGATAACTCCAGCTTGTGTCAAGTTGACACACAAAACCAGCACACACACCTCCTTCCTAGGTCTCCATGATATAAACAAA
GGGCTATTGTGACCTCTGTGTGTCTGTGTTTCAGGAGGGTGAACATTACAACTTATTTCTTTTAAATGTATGTTGGTTGCCCACCTCATATGGC
TGATGTGAAGACTAAGTTTTATACACATCCCCCACACACATATCCCACACATGGTAGTGTGTCATATTTGCAAGTGGTGTAGCATCTTTACAGC
ATGTCTTTCATGCACCCATATTTTTGTTGTTTGTTGCATCCCAGGAGCTCCTCCATCCCTGAGCATCCCCAGAGCCTATGTCAGAGCATAGGCA
GGGCTTTACCTAGAGGCAGACTTAGAGAACGACATGCAGGGCAAAGCCAGGCAGTGAGAATGTGTGGGGGAGGCAGCACTGCTTGCAAGATGGA
CCTCAGGCTTCTCCACTCATCCTGGAGGGGGGCAGCAAAACCCAGAGAGCCCAGAGCAGTTGGTGGCCTCCCTGCAGATCTTCCGCCTTGGAGT
GCATCTATAGCATCTATTTTTAGATTCAGGATAGCTCTAACTTCCTCCAGTCTTGTAGTCCAGACATTCAACATAAAATACGAAGACAAAAATT
ATACCCAAGGGGGCTTCCTGGGCAGATATGTTACAGAGAGTTTCTTCATTCATATCTCTGCCCCTTGGAGGGACGGTTTGCCTTTAAGTATGGC
GCCCGTTCAGATAACCCTTCTGTGAGGAGGGTGGAAGGGAGCTCACTGAACAGGGAACTCCTCTGCTCTGCAACAAGCACTGAGTCACAGATTG
CAAACTCATGGCTACTCAAGCTCATGGGCTTTGGGTTAGAACAGATCTGTGATCAAATTTCCACCATGCAGTTATTAGCAGGGAAGGGTTTTAA
TGTCTCCCAGGGCTATCTCCTCATTGCTATGTGGGTCTGATAATCCCTGTCTCACAGCATTACTAATAATTAAATGTATGACATGTACATTTAA
TGTATTTAATATTTAATACTACTACCTGGTATAAGGCAGTGCTTCCATACTCTTGATATTTTCAATACTGAAGAAACACCTGGAGCCCAGTTCC
CAAATCCACATGTCCCCGGGGACAGATAATTGTCACTGTGCATCGATAAGGAACTCTCAGTGGACAGCAGTGGAGGAACCAAGTTGCCCTGTGA
TACTGTCTGGTGAGGCTCAGGATTGAACATGAAGTGTTTAGTCCAAGAGAATCATCTGCAGGAGGCCAGTGACAGCCACAAACCAGAAGAAACA
ATGCCAGCTCTGATTAGACAACAGGGAGACAGGATCGAGTAGACTCAGTACGAGAGATGAGAACAGAGATAGCACCAGGATGGACGTAGCCTCT
AAATTCTCGTCTTTTAAATTATTTGTAGCTAAGGACCAGTTTAACAAAAAATAAAATCATATTTATCAGTGCTTTAATCTAACCATCCATGATA
AAGCTGGTTTGGCAATGTCAAGTGGAGGAGAGTGGGTCTCATTTTGGACACTCCTTTTACTGATAACTTTGCCTTCCATCTCTAGTGATTCGGT
CACCCACATTGTGGCAGAGAACAACTCAGGGTCAGACGTCCTGGAGTGGCTCCAGCTGCAGAACATCAAAGCCAGCTCCGAGTTCGAACTCTTG
GACATCTCCTGGCTCATCGAATGCATGCGAGCTGGGAAGCCAGTGGAGATGATGGGGAGACATCAGCTTGTTGTAAGTGTCTCCTTTGCTGGGT
GGCCACTGGCATGGGCATCAGAATCCACAGACTTGGGTCGATTACAGTCTTTTCTCACGGAGTTCACTTAGCTCTGGTGGACCTCCATTTCATC
CTCTTTATATAACAATAATGCCCACCTCCTGAGATGCTGTGGAAATCGAAGTAACATGCATACATGGTGCACACTGCTGACATATTAATAGATG
GCACTATTTTCTTTAAAAAAAAAGAATTTTTAGTGTGTGTGTGTGTGTGTGTGTGTGTGTCTGTGTGTGTGTATGTGTGTGCACATGCGTGCAG
GTGTCCACAGAGGCCAGAAGAGACCAATAGATCTCCTGGAGCAGGTTGTGAGCCACTCAGGTGTTTGGCAGGAGTAGCACTCAGTCTTCACCAC
TGACCCATCTCTCCAGCCCCACAACAAGAGCTATTTTTATGAACACATTCACACACCGATATTTCAGAATCTCAGCCTGGAAACTCAGAAAAGT
AAAGAGTTTCTTTTTTTTTCCCCATTCTGATAATTCATGAAAAAAAAAAGAAAGAAAGAATATGACTTCTTAAATCTAAATCTTTTACCAATAA
TGCTAGAGTCTCTGGGAAACGGGTCTGGATCATCTCTAATAAAGGAAACAAAGGAAATAAAATTTAATAGCTGGTTCTTTAAAAAAATTTTTAT
TGGTTATTTTATTTATTGACATTTCAAATCTTATCCCCCTTTCCAGTTTCCCCTTCACAAGCCCCTTGTTCTCTCCCCACTACCCTACCTGCCC
ACCCACTCCTGCCTCAGTGCCTTAGCATTATCCCACCCTGGGTCATTGAGCCTCCACAGGACCAAGAGGCTTCCCTCCCATTGATGCCCAATAA
GGCCATCCTCTGCTCCATTTGCAGCTGGAGGCCATGGGTTCCCTCCATGTGTACTCTTTGGTTGGTGGTTTAGTCCCTGGGAGCTTTAGGGGGG
GTTGGTTGGTTGATATTGCCGTTCTTCCTATGGGGTGCAAATCCCTTCAGACCTTTCAGACCTTTCCCTAACTTCCCCATTGCGGTCCCCGTGC
TCAGTCCGTTGTTTAGCTGCATGCATCTGTATCTGTATTGGTCAGGCTCTGGCAGAGCCTCTCAGGGGACAGTTTTAACATGCTTCTGTCAGCA
AGCACTTCTTGACATCAGCAATAATGTCTGAGTTTGGTGTCTGTAGATGGGATGGATTCCCAGGTGGGGCAGTCTCTGGATGGCCTTTCCTTCA
GTCTCATTTTTGTGATTTCAAAAATATATATCCTGCACAGACTTAAGATGTCACAATGAAATTCATTATCTGGTGTAATTAATATGTACTAATA
AGAAGAACAATCATACACGCCTGTGACTATACACTAGACAGGTATGCAACACATTTTTGAGTCCTCTATGATTTTGTCCTCTTTCAGAGACAAT
CGTCATGAATTGTCTTAAACGTTTTCTTTATATTTGAGTAAATGTTTATAGTTATATGTACACTTACTATTGGCTAATTAACTGTCATTCAAAA
TGGATGCTTATGCTTTCGTGTAGGTGAATAGAAACTCCTCCCCGAGTCCTGTCCCAGGCTCCCAGAATGTTCCACCACCTGCTGTGAAGAAGAT
CTCCCAATATGCTTGCCAGCGAAGAACCACATTAAACAATTACAACCAACTATTCACGGTAACGAGGTTTTCTCAAAACCCAGGGCAAAAGGGG
GGCTTGAGGTTTCTTTCCCTGAGTCGACAAGTTAAATTATGATTTTCTCGAGATCTCAGAATGCTTCTCCATGCTTACCCACCTAACCTGACCT
CTGGTTCTTCGTCAAGTGGGCCTCCTTGTTCATAGACTCCCAGCTACTCAGGGGTCAGGGTTCATCAGAGTAAACACACTCAGGAGAGTGTCAA
GCATCGGACTTCTCCTCTCTAACTTTCGAGCTGCATCTGTGAGGAAGAGCCGGAAACGGATGGCAGCCACTGAACCCACTGAAGCACAGCTGCG
TTCTCTGCTAGGAAGGCAACATGCCTCACCTTAAATAACCCCGGCTCTGGGTCTGAGTGATGAACACAGCCTTCATCCCACAAACACCAGCTGT
GTTTTCTGACCACACTAAGAGACCTGAAAGTCTCAGGAGTGTGACAGAACCCTGGCTAAAGACTGGTCCAGAAGATAGGAAAGGGTTATACCCA
ACCCAGTCATGACTGCTGGGACACCATGGATCCCAGGTCACTACTTGATTAAACTGTTTCTAAATGTCTAATCATTGAACTCAGTTTCCTCATA
AATGCATTAGATACATATCTTTTCAGTTCAGTGACAGAGTCCCACAGGGGAGGATGCAAAGGTACAGGAGCTTGATCCTGACTCCAAAGGCGCT
CCATTAGTAAAGGGCACAGGAGGGACTTGCAAGTGACTCCCACGAGCACACCTGCTGACCTCGTTTTATTGTTTTAGGATGCCCTTGATATCCT
GGCTGAAAATGATGAGCTTAGAGAGAATGATGGTTCTTGCCTGGCATTCATGCGAGCATCCTCTGTACTGAAATCTCTGCCATTCCCCATCACC
AGCATGAAAGACACAGAGGGGATTCCTTGCCTAGGGGACAAGGTGAAGAGTATCATAGAGGTAAGGGAGAAGTGGAATTTGCTACATCTCCAAA
GAGGTGAACGGGAGACAGAACAGATCGCATAAGTATGGGCTCTCTCCTCTATTTATCTATAAATGGATCAATATAGCAAGAAACAGATATATGG
GTGTAGACATATAAAATCCATACCCAACACGATCCCAGTAGGAAGACTTTCTCGGGCAGTGACTTTTACTAGCAGCGAAAGACTCGATCGAGCC
TTAGATCTCCAGAGCAGCAGAATATTCTGCTTTCCACCCCAGTCTGGGTGGGAGACTCGGTCTCCCTCAGCCCTTCCTGTACAGCACAGGGTGA
TGTTCAATGGGCTGATTGTGCATCTCTCTGTTCTCAGGAGAGACTGGGCAGTAGTTACATGCTTAGCAACATTTAAGGACTTCCCAACGAGGCA
CCGGTGTCATCTAGTAGCAAGTTTCAATCTAACCAAATGCAAGGTACATCCTTCTAGAAAAATATCGGAGCTCCAGACAACAGAGACCAGACCT
CATAAGGACTTGATAATGGCCAGCTTTTCAGTGGGCCAACAATTAACCCGGTGGTTTAAAAAGCCCTCTTATTTCCACAACTCGAATCAACTGA
AAAGAAAATGACCAGGGTGATTTGAACAATGCTTGTCAGAGAATATTTTGTCTTAGAGTCTCTAGGTTCAGATCTTTATCTTTCTGTATCCTCG
TCAGAGGTTATGAAGGTCTAGGATCTATGGTAAACCAAGATTTTTTTTCAAGATTACAAGGAAATTCTTGCTTTCTCCAGATCAGCCAGGGAAA
AAACCCTCAAGTTTCAGGACAAACCAGTAGTTGGGTCTTGCGCGCGCTCGACTGGCCAGGAAGAACGACGCTCCAACAGGATCCTTCTCCACAC
GTTTATTGGGAGAGCTTGATTGTAGAGGGGAAAAGACTTCGAGCCCAGAACTGGTGCTGCTTTTATAGGCCTAGGAGGGGCGTGTCTCACACCC
GGATTGGTTATGCACTAAGCCTCATTTGCATCTTCCTCATCTGATTGGCTACTCTCTCTCAGTACCTTACAGAACCTCATTATCATACCTCATT
TGCATGTCTCACATCTGATTGCTTATACTCTCAGTACCTTACACAACCTCATTATCATGCCTGGGCCAGGCAGTGTCTTTGCAAAAAACTTTAC
TGCATATGTACACATTGGTTGTTTGTCCAATCTTATGCGTGGTGGCCAGCAGTAGTCAGTGCCACTCTGCAACGGCACATGTGGCTTCCCACAG
TTGGGGCTGGGCTGTCCGTTTGAGACTGGCACTTTGATTATGCAGAGACATAACTCAAAATCTTTTGTTCAACCCTCCTTGCAGGGAATTATTC
AAGATGGAGAAAGTTCTGAAGCTAAAGCTGTGTTAAATGATGAGCGATATAAATCCTTCAAAGTAAGCGACTGTACATTTGCATACTGGGGCGT
GTTTTGTTTTATTTATCCTGTTGTTTGCTGTCGATGGTTATATAACTCAGGCTGCTATACCAAATGCCACAGAGTGAACTGATTAGTCATAGAA
ATTTATTTTCTCTCAGTTCTGGAAGCACAAAGTTTAGGTTAACTTTCAGTGTAAAGATTCTGTGGAGGACTTTGTGGTTTTCAGTGGCCAGCCA
CCCTCTTGCTGCTGCCCTGACATAGCAGAGATACAGATGGACAAATGCTCATTCTCAGCTAGCTTTCTTTATTCTTACATTATTGTGTGAAGCA
CGACTCCTTGCTTAGGGAATGGCACCTTCCACAGTGGGCATCCTACATTAATTAACACACATGCCCACAGGCCAAGGTAGTATGGATAATTCCT
CATTAAGACTCCTTTCCCGGGAGATTCTAAATTGTGTTAAAAATTAAACTAACCAGCACATCCCACAAAGACACTACATGCCAAGACTTACCTA
TCACACTGAGACTACCTACCAAGAATCTGTCACTCATGGTTACAAATGTCAAAGGGACACAGGATTCAAATAGAGCAAGTGTGGTCCTGAAGCA
CAAACTCTGTCTGAAAATTCCTCATTGCACCTCAATTTCTGGGACTTAGGGTTCACCACCCTCCAATTTGTTTTTGTTTTTGTTTTTTTACCTT
AAAGTTTATGGTACTATGGCCAGTCCATTCAGTTCATAAGTGATACAGGGCCTAACATACGGCCAGAACAGGAGCTAGGCTCAGAGTACAGACA
GACCAGGGTTCAAGTTTGTACTTTGCTGTTATCCACTTGTAGATCGGGTGAATTTGCTGAGGATCTTTGAGCAAGATTTACCTCATGAGTCTCC
TTCCACCAAAGGTGTGGTTCAGGACTTCATGTAAAAGACTGCAAAATCTGAAGACAGACAGACAGACAGATAGACACCTTCTCATACAGACACA
GAACAGGCTTCTTCTTACACCGATGCCCAGCCATCTTTCACCTGTTTCGTTTTGTGACACTTTGATTATGGAAGGATCCTTCAGAAACAAAAAG
CCTCTCAACGTTACCATCCCCAGGCTCAAGAGAGAACTCCCTAAAGTGCAGACAGACTGAGCAAGAGTCCCAGAACCAACAAAAAAGCTGGGTG
TGGTAGCGCACCTTTTAAACCTAAGCACTGAAGAGTTAGAGAGAAGCAGAGTGGCCCTAAGGCTCACTCGCCAATCGTCCCAGCCTGAGGTTCA
CTTCTGGTTTCCAAATGCACAGTTATGCATGTCTGTGCAAGTACATACATGCGCGCGCACACACACACACACACACACACACACACACACACAC
ACACACTCACTCACTCCCCATCCCCTTGATGTTTTCAAGAGAAACTGGGCCAAAGGTTTACTTATACTTCTTGTTCCTGGCCTGAGACTTTGGA
AACTGTCGCTCTGTTGTCATACCAACGATTTGGGTGGATAACAGCCTACCTTTCCCTCCCTTTCAGCTCTTTACCTCTGTGTTTGGTGTGGGAC
TGAAGACAGCTGAGAAATCGTTCAGGATGGGTTTCAGAACTCTCAGCAAAATACAATCAGACAAAAGCCTGACGTTTACACAAATGCAGAAAGC
AGGTAACGGTCTTGGGGAAGTCACGGGTCATTCTGGGCTTGTCAGGGTCCAGTGACATGCAAGCCAGTTTGACTTGCTCCAGACTTGAGACTCC
GGTAAATCTTGGCTCCAGCACTTGTCACTGAGTGATCTTGAGTAAGCTAGTTAAAACCTTGAGCCTTCGTTGCCTCACTGGTGAACCTGACCTA
ACCAGGGACTAGATCAGCTTGCGCAAAGCCCTGAGAATATGACAACAAGGCCTCTTCCTGCCGTGCTATGGTTCTCTTTATCCAGAAGTTACAG
CAGAGTGCGCACTGTCCCGATTCCTACAGACACACCTTAGGTCTGGCTGTCTCCCTGTGTAAGAAGCATGCATGAAGGGTCGAGGACAGAAGGG
TAAGCTGACGTGCAAGAAGGACAGAGGGGACTACTGAACAAAGTACACTCTCGACGGCTTGGTTCACACTCGGAATGTGCGCTGGGGGCTGGAA
AACGGAAGACAAAGGGGAGAAAGTCTAGCCACAGCCTTGATCTGTCCAATCAGAGCCCTAAGCCTAGGAAGAGACAAGAGTCCAGAGCAGAGAC
GGAAGAGCTCTCATTATCATCTGAACGTCTGGCAAGCCCCGCGGCCACCACGTAGGCCAGTTCACTCCCTTCCATGGACAGTGCTGGTACCTTT
TCCAGAAATGAGCAGATGCGATTCATAGCCCCTTCTAAACGTGTGAAAATTTTTTAAAGAATTATTTATTTATGTATATGAGTACACACTGTAG
CTATACAGATAGTTGTGAACCTTTATCTGGTTGTTCGGAATTGAATTTAGTACCTCTGCTCCCTCAGGTCATCCCGGTGGTCCACCATATGGTT
GCTGAGATTTGAATTCAGGACCTTTGAAAGCACAGCCAGTCCTCTTACCCACTGAGCCACCTCACCAGGCCAACATGTGAAATTTTTAAATGTT
TCCTTTTAATGTACAATAAAAATAGAGTAAAATTGTCCTTCAAGAGGGAGTCACTGCTATAGTTCCCTAGCTTCTGGGGAAATATGTGAAGTTA
TCAAGTTTTTTTAATATGGAAGTGATAGAGTCTTCTACCAGAGGATTCTGGGTATGCGCAATTATTCAACAGCTATCTTCTGTTTAAGTGATAA
TAGAAAGACAGACAGACAGAATGAACAAAATGATATCATAAGAAGTGGTAGATGAGGTGAATCATATCTTCGGGGGGGTCCCATCAATGACACT
ATAGAAAGCCCCTATTAATCTTATAAGCCCACTAAGTTCTTTATAAAAACATTTTTATTTATTTTTAGTTTATCTATATGGGTGTTGTCTGTAT
GTTTGTGTATGTACCACATTTGTCCAGTGTCCAAGGAGGCCAGAAGAGGGTATAAGCTCCCTTGGAACTGAAGTTACAGATGGTTGTGTGTTGC
TGTTGGTGCTGGGAATCAAACCTGGGCCCTCTGGAAGAGGAGCTAGTACTCCTAGCTACTGAGTCATTTCTCAAGTTTGATGATTTCTGAGTGA
CTATTAATAAGCTTTATATAATGCCACACAGGAAATAAAAGAGTCTAAGAAAAAAACATCTAATATTTCTACTCTGGATGCCAGAAAGTGTAGT
CTAGTAAGGGTGTTTTACACCAGCTCTAGAGATGCTGTTAAAGAGAAATGTATATGACTATCAAACCTAAGGGCATGGAGATGGTTTGGAACAA
TAGTGAGGACTCCTAACACTTACAGCAGAAGTGGGGAACAGTGGAATATTTTCTCAGAAAGCCTTGAACTTGTGATGTCCTGTTTCAGCATCTT
GAGTGCTAGGAATGCAGGTTGAGGTGTTGGCTGACATCCACACAGTCTTAAAAAGTCATAACCTCACATTCTTGCACATCATCTAGTGAGTGAG
CGGAGGGATTTCCTGGCACCACCCGAGGTGAACTTAAGTGCCAGTGATGATGGCAATGTCTATATATATTAGCACCTAAAAGAATGTTGCTTCT
TAGATCAAACACACACACACACACACACACACACACACACACACACACACACTCACTCACAGCAGCCATGTCCCAATCAAAAGGCAACTGTTGA
CAACAGAACAAGAGAAGTTTATGTCGTGTCAGTTATCGATATGAAGATGGCAGATCCCCCAAGGAACTGGCTTGGTAGTAACAAGTGATAGGTG
GATTAGCATCACCATCTTTTCCTGGTCCTGAAAACACAGTTCTGCAACTGTCCCGTTTAGAGATTACAGATGACTTTGAACTTGGTAACTGTTT
ACAGTGTGCTCAAGCCAAATGACTTTCTGGTCGTGTCTCATCCTGGGGTGAGAAACAGGAGCCAATCAGCAGCCTCTAGAGAAGTACATCCCCC
TCCTGTTTATCGAGTGGCATTTGGATCCCTTAAATGAAAGTTCAGCGTCTTATATGGAAGCATGACTGTCATCAGGCTCTGACTCTCCCAGACA
TGCCCAGCTTCCCACCAGTACTTTACCCCAATCCTTGGAGATAAGCCTCCTTCTCGCTCTGCCTCCCTCCCACTGCTCTGTGGTGACAGCCAAA
TGCTCTGCTCTCCCTCGTGACCCTGCTTCTGAACGGTCATTGGTTCATAGTCCTCAGGCCAGGAAGCAGCACGTCAGCCCCTCGAAGGCACTTT
ACACTTGGCTTTGGACACTGACCCACCCCTCTTCCTCCCACTGTCTTTTCTAATGAAGCCATCACTGCCAAGTCCTGAATTTTCCTACCCCCTT
TTAAATAGTATGGCATAGCTTGTTGGTCAAGAAGGCTGGACACAGTGCTACATACATGGAATCTCAGCACTTGGGAGGTAGAGGAATTAGGAGT
TCACAGAATCTGGGCCTTTCCTCAAAGGAGCAGCAGCAGCAGAAAGAGGAGGAGGAGGGGGAAGAGGGGAGGAGGAAGAGGAGGAGGAGGAGGA
GGCAGTGTTGGATTTTCATTATAATATCAGATAGGATCAATTTAGATTCCAGAAGGATCTGAATACTTCCCCCTTGATATGAATATTGTATACT
TCTACATGAGGAAGTCAAAACTCCTTTGAAACCATGAAAAAAACCCAAGCAAGTGGGCAAGGCAGTGCCATAGATTACTGGGCTCTGTACAGAG
TCTGGCCAATGCCTAAGAGCACTGTATGAGACAGCATCCTCTCCTATCAACAGCTGGTCCTCTTATCTGTGCCTTTCTCTCCACTGGCTGATCA
GGATTCCTCTACTACGAAGACCTCGTTAGCTGTGTGAACAGGCCAGAAGCCGAGGCTGTCAGAATGCTAGTTAAGGAGGCGGTTGTGACATTTC
TTCCAGATGCCTTGGTCACCATGACTGGGGGGTTCCGCAGGTAAGTGATGTAGCTGTTTTAGAACCCCTCTTCTACCTGCCTCTGAAATGGTGC
AAAACGCCTTTTCTGACAACAGAACCAACCTCACACTGTAGCTTTGGCTCCTGCTGCCTGTGTTTCGTGTCAGAGTCTCAGATAGCTTGACACC
CCGCACACAGAGCTGTTTCTTACCTCTGTGACTTTGCTCAGGTGAAACTTTCTCTACAGTTTTGTGAATTTGGTACTAATCTGACTTCACAATT
CCTTCAGACTCAATTTAGATGCTGCCACCTCCAGGAAGCCCTCCTCGACTCCTTATAGCTCTATTCTGGGCTAGGAACTTTCTTCTGTGCTTCC
CAGGTGCCTACTGCATCATGTGTGCTTTCCCCGTGGAGCTTAGTTCCCACGGAGACCAGGGTGTGAGCCTGACCACTAAGTAGATGCTCAAGAA
ACTGGTTGAGTCAAAGAAAGCTCAATGAACAAGCCTTCAGACATGAAATCTTCAGTTGGATACAAATCCCTCCATTTCAGAAACACCCTTGTCC
CTCCACCTCTCCTTTTGAGATCTTACCTGGTCACTTGCATCAAAGAGGTTTTCTGGGCTCGGTCTGTCACTTAGCAGAGTGGCTACTTAGTATG
GAGGAAACCTGGCTTTGATTCCCAGTATCCAGTAAATCAGGTATGGTGATGTAGACCTATAATCCCAGCACTTGGGAAGTACAGGCAGGAAGAT
AAGAGGTCCAGGGTCACCCTCAGCTTCAAAACGTGCTCAAAGCCAGTCTGCATCATATAAGACTCCATCCTTAAAAAAAAAAAAAAAACAAAAA
ACAATCATAAAGAGCAATCTCTTAACCCATACCATAGTTCCAAGAACGCTACCTTGTATGGTGTTACCAAAGCTCTTGGTTGGCTGCCTATTGT
CATACATGTTTCTCTATGTATGTGATGCATATAGAAGTAATATTTATTACTTGCTTTCCATCCATATACAACAACACACATTCTGGATGAAGTT
TTTTTTTTTTCTGTCACAAATTTGTTTTGCAAGAAGTGAGTTGAACAACCAGGAATAGCTGGTTTTAAAATGTTGTGAGTATTTTGAACTACAA
ACCTCACACTTCAGACACAATAACAAAGAGTTTTGTTTTGTTGTTTTGTTTTTAAATGAAAGGCTAAAAGTCCAACTGATTTTCCTTTTGTGCT
TTTTTCCCCCCAAACTAGGGGTAAGATGACTGGACATGACGTACACTTTCTAATTACCAGCCCAGAAGCCACAGACGATGAAGACCAGCAGCTC
TTGCATAAAGTGACAGACTTTTGGAAGCAGCAGGTAAGGAGGCCAGATGGCACCTGCCATTTAAGGCTGGCTTAGATTGATTTCTGCTTGTTTC
TTTTCCGTGTGTGTGTGTGTGTGTGTGTGTGTGCTTGTGTGTGTGTGAATGTGTCTGTGAGTGTGTGTGTGTGGTATCTATGTTATGTTTGATA
TGTATGTGTGTGTACTGCTGTGCGTGTGTATGATGTGTGTATGTGTGTCTGATGTGTTTGTTATGCATGGTCTGTATGTGACACGCACATGTGT
GCATATTTTTGCATATGTGTAGGCACATGGGTGCGGACATGTCGACCCTGAGTTTGATGTTGAAAATCATCTTCCGCTACTTCTCCACCTTATT
CATGGAGGCAGGCTCTCTCAATCAAATCCAGAGCTCATCGATCTGGCTAGTCCTACTAGCCAGTTTGTTCTGGGAATCCCTTTGTCTCCACCTT
CTGAGGCTGGAATTGCAGGCAGGCTGTCATGCGTACCAAGCATTTGTATGGGTTCTGGGGATCTGAACCTTGGCCCCCTGTGTTGTAAAGCAGG
CGCTTTAACCACCGAGCCATCTCCCTAGCCCCTTGCTAATCTTAGGTGTTACATGATCAGTGTACAGTGACTACTATTCTCACTTGTTTGTAAC
ACCTCATCGCAGCACCATGTGGCTGGCATGAGACTGACCAGCTTTGGGTCCTAGGCAACACTTTACGGGCATGCATCTTGTTTGAAGAAGGAAG
ATAAGGAGGGCAAGCTGGGCCCTGCAGAGGTCATGGCCAATGGCTCACACTGGATGAAATCAAACCTGCCACGTGGCAGCCAGTGGTTAATGGT
CAAAGCTGAGTTAAGTTAACCCCACAGCCAAAAACCTCTGGCTCTCTAGTCAAGCACCATTTCCTACAGACACCAAGGGCTCTTTCCTGGGATC
CTACCTGTCTGGCTGAATGGAGGAGGCTGGACCAGAGTTTTCCTAGTACAGACAGCTTTCCCCTTCCTGGGCTGAGCTCACTGATGCACCAGAG
AGATAAGACCAGATAAAGTGAAAACTAGAAGGGGCTGGAGACATGGCTCAGTGGTTAAGATCACTGACTGCTCTACCAGAGGTCCTGAGTTCAA
TTCCCAGCAACCACGTGGTGGCTCACAACCATCTGTAATGGGATCTCATGCCCTCTTACTGGTGTATCTGACAGCTATAGTGTACTCACATAAA
ATAAGTAAATAAATCTTTGGGCTGTCGTGGCACACGCCTTTAATCCCAGCACTTGGGAGGCAGAGGCAGGAGAATTTCTGAGTTCGAGGCCAGC
CTGCTCTACAGACTGAGTTCCAGGACAGCCCTAGCTATACAGAGAAACCCTGTCTTGAAAAAACAAAAACAAAAAAAAACAAAAACAAACTACA
AATAGCCACCCCCCGAGAGCCATGAGATTTATAAATGAAAGCTAGATTTCAGATGCACATCCAAATTCATCTGTGAGACCATTCCATGTCCCCC
CCTGTCACCCCTCCTGAGAAGTCAGGAGAAGGTGCACTGGAGAACCCCTTCAGTGATAGCAAGATAGGCATGACAAAAGTCAGAATACTTTTCC
AATTCTCGATTCTCTGCTCCAGGGGTTGGGAGTGGGGGACGTTCGGGGGGTTGGGTAGGGGAGAATAGGGGAAGATTAAAGACTCACTGAAAGA
CCAAAGAATACCTCCCCACCCCCAGGCCTGTTGCCAAGGCTGGAAACTCATTGATGTTTGTTTTTGTGCCATTGTCCTATGCTAAGACATCTGC
TGGACTTGAGAGGTGACAGAGGAGCCCTCTTTGCATCCCTAGTTTCAAGGTAGTTTTCCTCTGGACCCTGGGTACAGATAGTCATTAGAAGCCC
AGGCCCTGCATTCGGAGCCAATAGTAAAACAGGCCTAAATGAGACTTTAGAAGTGCTGGTACAACCTGGGGTGGTGCTTAACAGTTCATGCCCC
ATTTTCTGTCTGTGCTAACCCGGGATTCAATGAAAGTCTCGGCAATTCAATAGAAAATCTCTGGTGTCTGACAAATGGTGGCTAAGATCAGCAG
CAGCAGGATTGCTTATTCAATTCAGTTTGAGGGCCAACCACTGGGCTGCAGGGGACCCAGGAACCAGCAGGCCAGTTCCTTCCCTTCCCTCAAG
TACCGTGATTCAGTGTCCCTGCTCTGAATGACCCATCCCCCTGAAGCCCTGGACCCATCCCCAATAACTCAATTGTGAAATCAGATGAGTTCTT
CCCATCTGGCTCCCACTTCCACTTGACTCCCTTCCCTGCCAGCCTAACCCTGAGCTTCACTTTACAGATGGCCGTGAGCACAGCTTAGTACAGT
GCATACCGGTGTACTCACAGTACACACACAGCATGCCCACAAGATGCTTAAATGTCTGCGAAGTAGGTTGAACAGCCTTTAGTGCTTTCTCCTA
TCTAAATGGTGTCCTTTGGAGACAACCCCTGGGTCAGAGGTAGGAGAAAGGAGGTGAAGAGCTTAGAAATCTCATCTCCAGGGAGGTTGTAATT
CCCCCAAACATTTGTTCTGCCTGAATTCCAAGCAGACTGCTGGTAGCTCACCTCAAAGGAAGGAGAGAGACTTCATGACTCAGGCTTGCGGTCA
GGCCTGGTAAATCGGACCGGGGTACAGGGACCCTAAGTGCTTCAGGTGGTCTGTCACCCCTGGAGTTCATGGAACTTCTGGGAGTCAAAATGTT
GAGTCTACAGAGTAAGACTGTAAGTCAGAACCAACCATGGTGGCTCACACTTCTCACCTCAGCCATTCAGGAGGCAGAGGCATGAGTTCCATTA
CAGGATACCTAGAACCAAAAGGAGTTTAAACTGGCCATGACCTTGGGTTCTGAATTCATAGGTTTCACTGACCTCAGATAGGAAAAGATACTTT
AAGAAGAAAGAAAGATAGGAAAAAAGAAAGGAAGGAAGGAAGGAAGGAAGGAAGGAAGGAAGGAAGAAAGAAAGAAAGAAAGAAAGAAAGAAAG
AAAGAAAGAAAGAAAGAAAGGAAGAAAGACCCACACCTATACTACCTATACTGAGCATCCATAGGCACCTCCTATCGTCAGTCTCTAAATACTA
CACTGTGACAACACTGCATGCACTTATATAGTATTAAGTAATTAATCTAAGAGTGATTGAAAGTCTGTGGGGATGTCTGTGGCTCATATATAAA
CATTCTTACCACTTTGCAGGAAGAACGTGATCATCTGTTGCTACCGTGATGACTGCTAGACGACTGAGGGATAACTATGGGACAACTGAGGGAC
AACTGTGGGATAACTGAGGGATGACTGAGGGATGATTGAGGGATGATTGAAGCATGACTGAGGGATGACTGAGGGATCACTGAGGGATGACTGA
GGGATGATTGAAGAATGACTGAGGGATGACTGAGGGATCACTGAGGGATGACTGAGGGATGACTGAGGGATGACTGAGGGATGACTGACGGATG
ACTGAGGGATGACTGAGGGATGGCTGTGGGATGACTGAGGGATGATTGAGGGATGACTGAGGGATGACTGAGGGATGACTGTGGGATGACTGAG
GGATGATTGAAGGATGACTGAGGGATGACTGAGGGATGACTGTGGGATGACTGAGGGATGATTGAAGGATGACTGAGGGATGACAGGGATGACT
GTGGGATGACTGAGGGATCACTGTGGGATGACAGAGGGATGATTGAGGGATGATTGAGGGATGACTGAGGGATGACTGTGGGATGACAGAGGGA
TGATTAGGGAATATTGAGGGATGATTAAGGGATGATTGCAAGATGACTGAGGGATCACTGAGGGATCACTGAGGAATGACTGAAGGATTACTGA
GGGATTACTAATGGATGACTATGGAATCACTGAGGGATGACTGAGGAATCACTGAGGGATGACTGAGAGAATCACTAAAGTTGATGACTGAGAG
ATCACTGAGGAATGACAGAGGGATCATTGAGGAATCACTGAGGAATCATTGAGGGGGGAACTACAGGATCACTGAGGGATGACTGACTGAAGAA
TAATTAAGCAATATTGAGAGATGATTAAGGGATAATTGAAAGATGACTGAGGGATCACTGAGGGATCACTGAGGGATCAATGAGCGATCACTGA
GGAATAACTATAGGATCACTGAGGAATGACTGAGGACTCTCTGAGGGATGACTGAGAGATGACTTCCCCCACTTGAGTTTGAATACTAGATTGG
TTCCTCTGACGAGAAGATGGGAAGGAACTGAAAACAAAGGTCAAGAATCCTGCCTGGAGGACTCCTCATAGAGTCCTGACAACACTTCCTGCTC
TTACACCATTGCTTGGGGTAGCCTATGGCTAATTGGTCCACTAAGTTGGAAGAGGTAGATCCTCTTTCTTGTAAGAAGCAAAGCTGAACTGCCC
TAACCCCACCCTGATCCCTCCGACCCCCATACCCTGGGAGCAGAGAGAAGGAATGGCAGGCAGAGCTCCTTCCTACCCTAGCCACTGATCTTCT
AATAGGAAGACAACCCTGGGCAGCCATGGCCGAGCCTGAGCCCAACCTCAGAGCAGTTCCAGAGGAGATAAATCACACTGAGGGACCTGGGGAC
AGGAAAGGAAACTGCAAACAGTAGGGGAAACCCAACAGAAAGCTGGCCTGGAAAGCTAGGACCACGTCTGAGGTGGTACCAGGCTCCACTACAG
ACAGCACCCAGAATCAGATGGAACGCCCCAGTCAAAGCCTCGCCAAGTTCAGGATCAATTCACCCAAGAATGTCCCATCTGTCACCAGCCAAGT
GCTACCCCCCACCCCCACCCCGTGTGCAGCACAGGAACCCCTAGGGGCTGGTGCTTGCCTAACATTCCCTTGAATTACTTATTAACTTTCTGGA
TAGAAACTTGAGATAAAGCAGGCTTCGTGGGCCTTCTTTCACTGCCTCCGAGATGGAGAGAGAGTTCTTTCAGGGCTGGGGTGTACTCAGTGGT
AGAGTGCATAGCTAGCAGGTTCAACCCTGAGCATTGCAAAAACAACACAACAACAGATGTAACAGGTCACCATGCCCCACACTGGGTCTGGAAT
TTCAGACACAACCTTTTGACCCAGTCCAGACAACCGACATGCAGGGAACAGTGTGAATTTCTGTTCACCGTGTCTTTGCTTTTCAAGGAGCAAA
GTTGTCTCTCAGAAGCAAACAAGGAGAGATGGAGGCTCTGAGGTTTTCTTATTGGGGACTTTGAGCTCACCCTTTCTGTGGAGAGAATCATGTA
TTACCTCCTCTCCCAGATGCTGGATGAGGAAGAACAAAAATGCATCCCCATTTGTTCAGCTCTGAAGAGGCTCTCTTGAGAGCACTGTAATATC
ACATGGGTGCCCCACCCCCAGCCCCCTAGGACCCTTTCTAAACCCTCCCATTGCTTTTCAGGGGTTCCTTTTGTACTGCGACATCTTAGAGTCA
ACCTTTGAAAAGTTCAAGCAGCCCAGCAGGAAGGTGGATGCTCTCGACCATTTCCAGAAATGCTTCCTGATTCTGAAGCTGGACCACGGGAGAG
TGCACAGCGAGAAGAGCGGCCAGCAGGAAGGAAAGCGCTGGAAGGCCATCCGTCTACATCTGGTCATGTGCCCCTATGATCGCCGGGCCTTCGC
CCTGCTCGGATGGACCCGCTCCAGAGTAAGAGCTGTCTCCTACTAGCTTTCCTGGCTCAGCCGAAGAAAGCTTCCGCTGCGGGGCACAACGCTG
AGAGAACTAAGGGACCATGCCCCCAAGCCTCTAAACTGGGGCATGGTTTGGGTACATGTTCACACGAGTTAGATTCATTTAGATTCCCAATGTA
TGGTTCACCAAGCCAGCAGCAGTGGCCTTACCTGGGGACAGGTTAGATTTATAAATTCTTCACTGTATACCTACCACTACATCAGAGGCTCTAG
GCAGTTAAGCTCTGACACGCATGTTTGTAAAACACCCTAGGTAACTCACACGTATCTCAGGTCAGAGCTACTGAAACCTAAGAAGTCTCAGCGT
AGTGAGTAACTCAGATGGATTCCGGTCCTAGACCTACAGAAGGACCCAACCCTGCCTCTTGGACACCTTCCTAAAGCCTATAAGATAGATGGGA
GTGGCCCCCTCTGGGGGCAGCAGTGCACAGCTGCAGGGTGGTCGACTCAAATGCTGCTCTAAGAAACTAGTGAGGGTTAAAAAAAAAAAAAACA
ACTTTCTTTTTCAAGTTTCAAGAGGCTGAAGCTGCTCTGTCCCGGGTTGCGCCGCTTTCAGGTAAATAACACTCCTCCAGCCTTCTCTCTAACA
GCGGCCCTAACCTCACAGGGCTCTGTGGAGGACTCTCTGGGACAGCAGTCCATCCACTCCTCTTACATGTCATGCCAGGCTCCTGTTAAGCCTC
TGTTGCCCTGTACCCTCTGGGGCTCAGGAAAAGACTGGATTCCTTAGTTAGGGTTATGATAGCTGTGGCAAAATACCACGATCAAAGAAACTTG
GAGACGGAAGGGTTTATTTTACTGACATTTCTTTATCACTGTTCATCATCAAAGGAAGTCAGGACAGGAACTCAGGTAGGGCAAGGACCTGGAG
GCATGAGCTGATGCAGAGGCCTTGGAGGAGTGTGGCTTACTGGCTTGCTCTCCGTGGCTTGCTCAGCCTGCTTTCTCATAGAACCACCAGCCCA
AACAAGGTACCATCCACAATAGACTGGGCCTTCTCTCATTAATCACTAATTAAGAAAAGATCCTACAGATGGATCTTACCAAGGCATTTTCTCA
ATTTAGGTTTTCCCCTTTCAGATAACTCAAGCATGTGTCAAGTTGACACAAAGCTATCCCACACAGCACAACTCTCTTGGCCATTCTGCAGCCA
TTTTGGTTTTTAATCACCTCCTTCGAGCCCTCGTTCAGTGATGCTCACCATGGAAGCCCTGCTCAGTCTGGATGCAGCACTGGCTTCCTATCGG
CCCATAGTTTTCTTATTTCTTGTTATTATAAACCATTTGTGTGCCCAGCTAGATTCTGCTCCGGAGGAACTGGCACTCTGCAGGTATTTGGCAG
GTGAACTGAATTTCTCTCTGCACCACAGAAACACTCTTGGCCTTGGCCGACGAGTTCAGGAATGTTTCTCACTCCCAAATCACACAGGGCAGGG
AACCGGGAGTACCCCATCATTGACTGCATATAACCAACCCCCATTACACCTCCAGCCTCTCCACTTGCTGGGAACCAGTGTTAGCTGTGGGAAG
ACACTGCCAGTCAAGAGCTGGGGTGAGCTAAGCAGGACCCACAACTTCCCAGACCAGCGCTAAGCAAATCTCCTTCCTCAGCAATTGTTACACA
AAGCTGGTTCCACCTACAGCTGCTTCTGTAAAGGGCTTCTGGAGGCAATGTCTCACCCAGTCTCAGAAACTTTCCAGCAGAGAGGAAATGAATT
CTTACTCCTAGAGGGTGCGACTGTCCCAATGTTTTGCCTAATCCCAGCCGGAAAGACAAAGTTCTGGACCTGGGAAGTTTCCAGGTTTCTCTCA
GGAAACCAGCAGCAGTATTTGTGATTCACTGTCCTTGTTTCTAGTGTTTGCTGGTCACTTGTCCTAGGCTTCAGATTTGCTCCCTGAAACCTCA
GGGTATTCTTTTGATCATGATAGATGTCATTCCGGGGCAGGTGACTCTTGACAGAATCTCAGATTGAGTGGACATCAAAGAAGAAAGGACCTTA
ACGTTCACTCCCTCCACCTTGGGACAAAGCAAGGTTCAGAGAACCTACGTAGTAAGGACACATAGATAGCCATAATATCTGGGCCAACATCTCC
CCACTCCTCACCCTCTATTATAATAATGATATTTGAAACTTAAAACGATTTAGGTCACATTTTCCTCTGGACCAGGTATTATTCTAGAAACAAC
GCTCCTACTGTCCATTTATCCACTCAACAGATATTCACACATCACCTCCCGCCTGTTGGGCAGTGATTGACAAGGGTCCAAGCCAATCAGCTTC
TTGTTCACATTGAGTTTCTGTTCTAGTACAGGGAGCCCAGAGCTCAGCCACCCGGGAGCTTTGCCCTGAGGAAGAAAGTCACCCCAAGAATTTA
TGTTAGAGACAGCAGTTTCAAACACCCAAGGGCTTCGGATAGCTCTAAACACCATGTACCAGAAATAATCTGGACTGGAGGGTAATTTGTTTTA
ATTCTCTTTGTAGCAGTTTGAGAGAGACTTGCGGCGCTATGCCACACACGAGAGGAAGATGATGCTGGACAACCACGCCCTGTATGACAGGACT
AAGGTAGTGTTCCCTTCTGGGGATGGGTGACTGACCTTTACCTGTAAGCCCTCCCTTTGAACAGATTTTCTAAAACAATATTCATTACACTTAT
TTATTTTTTTTACATTGGGGAAAATGGTTGCATGTAGAGGTCAGGGGAGCCAGTTCTTTCCTATCACATAGGTTCTAGGGATGGAACTCCGGGC
CTCACGTTTGGCAGCAAGTACCTTCACCTGCTGGTCCCACTTATCCCATCCCTACCCCCAACCCAAACTGATTTTAGTGTCTAGGTTTGCTTCT
TTTTGCTGCAAATGTTCTGTTCTCTGTGGATCACAGTTGAAAGGAGCTGGAAACATTGCTTCAACCCCTACTGGGCCCAGGGTGCAACTCCAGG
CTTCTACTTCAGAGAGTGAAGTAGCAGGGCTCTAGAACCAGCACATAGGTGAGCAGAGCTCAGCTGGCCGTGTTCCTGGTGTTTACGGTCTTTT
ACTCCAAAGAAAATCAGGGATTCCTGTAAATCATAAATGTCACAGGAATAACCATCCAGTGTAGGGGCAATCACCTTCTGAGGGGGTCATCAAT
GGGTGTCTCCCACAGTAGAGCGACACAGGTGCCCACAGGTGGATGGGAAGCCATCAAGCCCCAGTCACTAACCATGGGGGACTTGAGTGTCAGC
AGCATCTCATTAAAGCCACGCCTCACAAAAGTCGCAAGTTACCATGTGGCTGGCTGATGAAGAATGAACAATTCTATCGTGAAATCCATGAACA
AAAAGGTCTCCTGTTCTTCAAGTGGTCAAGGGCACCAAATTAGGCTTGGAGGGAAAAGAAGGTATAAAATCACCAAAAGCAAGAACTGATCTCC
TTGCCTCCTAGGGGTTTATTATAGTTTAAAAGAAATGGATATGCATTGGATATCTAACACTTAACCTAGAACTTTAAATAGACTGACAGCAAGG
CCTCTCTGGGACTTTTGACTTCATCAGACTGAAGTGTGATTCAAATTTCAATATATTTTATTGCGGGGGGGGGCTGGGCAGGGAGACAGAGACA
GAAACACAGAGAGAGAGACAAAGAGAGATTCAGAGANNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNCCCAGGAAATTGT
AAGTGCCTCAGTGAGAAACCCTGTCTCAAAAAAGTGAGGAGGGGGAAATGTTAGAGGAAGGCATCCACTGTTGACTTCTACCCTCCATATGTAA
CACGCTCATGCACATATACCCCCACACATATGCGTACACATGCATAAACATGCACTCACATACACACACAAAGGAGAATAGACACTCAGGGAAA
AAATATCCACGTGTCATTTGCTAACAGGGGAAGACTGTCACGATTTCACCTTTGGATGGGAAAGTGTCAAAACTACAGAAAGCCTTGGTCAGTT
GGTGGTTGGNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNCTGGCTGACTGGCTGGCTGGCTGATTGGCTGGCTGGCTGGCTGGCTGGCTGGCTGGTTGGCTGGC
TGGCTGACTGGCTGGCTAGCTGGCTGGCTGGCTGGTTGGTTGGTTGAAATTAAGGGCTCACTCTACAGCCCAAGCTCTTGCCTCTGCATGCCAA
GTACTGGGATTCCAGGTCACCACACCTTGACTTCTGACCAAAGCAGGGTGGCATGCTTTAGCCACCACTCATTCTAACAAGACACTTCAACAAG
TCTGCACTGAGGTGCCAAAGGAAAATGCCTGAGAGTGTGTTTCTCCTTGTGCTTCCAGAGGGTGTTTCTTGAAGCAGAAAGTGAAGAAGAGATC
TTTGCCCATCTGGGACTGGACTACATTGAACCATGGGAAAGAAATGCCTAAGACGGAGTTGTTGACATTTTTCTTTTCGGTTTAGACAAGCTGT
GCTCTGTGTTAGTAAAAGATGGCTTGGGAAAGTCTGGGATTCTTTGACCTCAGAGGATGCTGGCAGCAAGTGGATCATAAGTATACTATGCAGA
GAGAATATTCTAACGCAACTTTGAAGTCTTGATAAACCAGGATGCAGCCATGGTTCACATTCTTGGGCCATCTGTCCTACTGTTGCATAGAATT
CATAGTCTTTTGTCTGCTTTGTTTGAATAAAAATGGTCTTGTCATTGCAGCATCATCTAGAGAAGCGCATCAATGCCCACCTGACTCCTGTGTG
TCTGAATCTCTGTGTATTGCCAATAAAAATAGGAAACAGAAGCATGAAGTTTTCTTTGCTTTGTTCAGTTATTTTATTTCTAAACACAAATGGG
AACGTGGAAGAGTAAAAGCTTGCGGTTGATTCTGAAGGCATGCATGGGTGTAATTAGTACATCAGACGTTCCCAAGAGGACCCTGGGCCCCCTC
AGAGGTCCTGGGAGGCAGGCTGCTGATTCCTAGCTGTATCCACTCTCCTCTGAGCTGGTTCCTTTCGCACCCAGCCCTGCAATGTATCAGGGAA
GTGTTCTCATTGCTCTCTCTTGGATGCAAAAGCAATTTCCTAAACCATACAGGAAAGACTTCCTTTTCCTCCTGGAGCTCTTAAAGAGAGCGTA
GGTTTCATCTCAGGGAACCCATGGGAGGAACTCAGTCTCTGCAACAGCCCTCACCTAATCATTCTTACTTCCCATGTCCTCCAGTGGGGCACAG
GACAGATTCTATGCCTTGAAGGGACCTAAAGGCCACCTATTGAAGCAATATCACTGTCTTCACCCTCCTGTAGAGCTGGAGGCAAACTGATGAG
TGTCAGCTGTTCCCAGGGGATGAGGGCTGGAAGGAAGTTGTGCCGAGGAAGGAAAGTGATGCCCCATGAGAAGCGAGGTCCCTCACCACACGGA
CCCTCGTAGGATTCTCCTAGGAGGACAAGAGGAATCCGATGTCACTAGTTACTGTGCTTTTCCCCATAGAGGGTTAACCAGCCTGACCCAGCTG
CCTCTTCTCCAAGAAACGCTTTGCCATTTCTTAAGGCCTGGTCAAAAAACCAGGGTGGCCTTGGGTTGCAACAGGACAATCCGGTCTGCCCCAA
AAGATTCGATTGTGTCATACTACTGCTCCTCTAGGAAGACACAATCCATCTGGGACTGTCAGTCACTTGGCCAAGAGGCCACCCCCCAGTAAGG
TCCTTTCACCTAAAACTAAGGTTTTGGGAGGAATAACTAACTGTCCTTCTAAGAAGATGGTGTACTTCCCCCGAGCCCCGAAAAAACCACCCCT
TCTGTGAAGTTGCTTATAGAACTGTTATTGCAAAGCTTTGCCAACCCCACCTGCTTTGTGAAATTTACTCAAATAAGCTTAACTTTTCCTTTTC
TTAAAAAAAAAATCTCTTTTTCATACTTTTGGATATAGGCCAGAGCCTAGGCTGTCTCTGATGCTCTGGCCTTTAGTGCTTTTTTCTCTGAAGC
CATATCCTTCCACTTACCAAGTGAAGTGACTGCTTGTCTGACATCTGACTGCAGCCCTCCATGCTTAAGCAAAAAGCCCCGTTCTTCTCTCTTC
CTATATACTTCCTCTTCTTCCCTCTCTTCCATGCATCTGGCAAAATTTAAACCTTTTTTTCTTTCTTCCTTTTCTTTTCTTTCCTTAAATGCTA
ATAAAGCTATCCCAAAGCTGCTATCTGAGCTATTTTTTTTTAACCTTTCATTATTAGGCCCAAGAATTTCAAGAGCATAACCCCAAATTCCCTA
GTAACATCAACATTGCAGCAGAACACCCCAAGGCTGGGCTCGGGCAATACTGGGGGAGATGAGGCTTCAATCTGTAGCACAAAAAAGCAGCCCA
CGCCCTTGGGAAGTGAGATTCGCAGCATCAGTTTGACCAGAGCCCAGGTCTCACGGGCATCCAGCCTCAAGCCCAAAGTCTGGAAGCACATGGG
ACCCTGAAGTGAACGTGTACATGGGGCTGCTTGAATCCAAGCTCCTCCCAATACCTTAAGGGGTCTAAGAACAAAACCCTCAGGGAACCCAAGG
GTTGTCTAGCAGGGTAATAGGACATTCTGCCAACCCTTAAGGAGACAGGCCTTCCCACTGCTGGTGATTGACAGATGCCTCTCCAGCCACTTTA
GCTCTATCCATTACACCTTTAGAGTCCCATGCCACAGTCACTCAAAGGCGATCGCTCAGCACCTCTCCATGCAGGTATGTGCCCCAGTCATGGG
ATAGTGCATCAAAGGTTCACACATGACCTCAGCAGCCTGTCGAGTGGTTGAATGGCAACCAACTACATGGCATTGGCTAAACCAGTGCTCCCAG
ATTCTCCCTCATGTCCAAACATGGGCATGGGAATCTAAAGGTGATAAGTCTTCGTACCAACAGAAGTCCTGAACCTTGCAAGTTTAGTGAAAAC
AATTTGAAATGAAGATTCTAAGCAATTGATTGTAAGGAGAATTAAAATGAAGAACACAAAAACAAAAACAAAAAAACAAAATAGTCATACTTTA
TTTTATAAGTTGCAAGGCTTGCTGATCTGTATATTTCAGTCACGCCTCACAGCTCTCCTCGGGGATCAGTTTTATAAGTATAAAACTGAGGCTC
TAGGTGGCCAACATTCTAGGCTGGAGCTGAAACCCATATATGTCAAAGCCATAGTCTCTTCAAGTTCTCAGGGAGTCTAAGGTCATTACCCATC
ATGCAAGAGAAATGAGGCTTCATCCCTGGGCACAGCTGATTGAAGATGTTCAAATGTGTTTATCTAAGACCTGAGCCCATGAAATAACCCAAAA
TTGAATTTCAGAGGGGCTTTGAAAGGCAGCTGTTAACATGTGATGGTCATTGGGTTTACAGAACCATTCCCTATAGCTCAAGAAATCCAACTAT
TTCTAAACAACCAGAGGCCCCATGCAAGAAGAGTTGGTTCTCTGGGATATCAAGGTGGCTCAACAAGACCATGGATGTTTCTAAAGATGCCCTC
AGGCCTCAGCTCTCCCACTGTGAAGCATGGTGTATTTCAAGGCAGTGGCTGTGCACTAGAAGGTCACAAGGAAACTGTCTGGGGAAGTGAAGGC
AAAGATCAGGCCCGAACTGGTGAAGTTGTCCCTAGATGTCCCTTGCATAGCAGGTGTAGCTATCCCAACCCATCCCTTTGCTCCTGTGTTCATC
TTTGGTCCTCTTGATGCCTCTCCCTCACAAACATCTTTTCTTTATGGTACTTCAAAGCAAATGGCTCATTACAAAGAAAACTCATTAAAACATG
AATATCAGTTTTATATTAGCTTACTTCTCTCAGTGTTATGATAAAGTCAACTTAAGGGTGGAGGTATTTATTTTGGTTCAGTCCAAGAAAGGTG
TGGCAGCAGGAGTGTGGGGTAGCTGGCCAAACACTTTATCTGTCGTCAGGAAGGATAGTGATGAGTTCTGCATGCTCAACTTGAATTCCCAGAA
CCCCAGGCTATGGAATGATGCTGGGCCACATTCAGGTGGATCTTCTTACTCAGTTAAATCTTTCTGGAGGTGTGTCTCCTTGGTGATTCTCAAT
CCCATTGAGTTGACAGTGAAGATTAACCATCCAGGACTCGACCATCCTAAAGGTTAATATGTAATGTGTAACACAGATCTCTGTACTTGTGATT
TGGAACTTGGTTGACACCCAAGGGGAAAAAAATGGTGTGCAGAGTCGAAGTGAGGAGACTGCCAGTGACCACTAGAGTTATTTTTAAATGTCAA
AAATTCAGATTACAGGCCACAGAGAAGGTTTAGATTTCCTTGTCCACTTTGTGACTCTGGATGGATGATAAGTAGGATCGGAAGATGTGGGAAG
AACTGGGTGAAGAGGATTGATATCTCTAGTGTCTACTAAGTGGAAATTGCTTCCTTTATTCCTGCTCTAATTTTTCCAGTGGCCAAAGGAAGCA
CAGAACCCAAAACAGGCTCGGGGAGGGTAGATGAGCTCATCTGACTTCTCAAAGTGCTCAGCAGCACGTCTGACTAGGGAGTGCCTAGCTCATT
CTCAGCTGTATGACACAAATCAGCCACATACTTCCTTGTGTCGGGGCATGGACGGCTCCATGCCACCTTTATCCTGAGATATTCAAAAGGAAGA
AATGGCTTCTGTCACATGTCTTTGGTTAGGTTTTATGCCTGTCCTAACTTGTGCCAAGCTGGCCTTGGTCAATGCACCCCCCAAAAAAGCCAAC
TCTCAAAGCTTTATTATTTTTTAAATGATAACATTTGGAGCAGCTAGAGGAGAAGGCAGGACACTCGGGAGCCAAGCCGTTTCCCAAAGGCAGA
CTTCTCTCGATTCAGAAACAGCACATACGGGCCTCATCTGGACACATAGTCGACTCACCTGCGTCCCCTTGCTGATTACCCTGCAAGGTCTGAG
ATCGAATGAACCTGCCTGCAACCATCCCCTAAAGGGGCAAGGCAGCCAGATGTGTCTTGGATCTATAAATGAAATTGATGTTGAGTGACAGCCA
GCTGACTCCTCGCCTCTCCAAGCATGAACCCTGAGCACAGATTCCAGGTTTTCAGCCCTGATCCAGCTCTGGGTCTGCTCCCTGATGCGGTGAC
ATCATTCTAGGCTCAGGCTGGGTACAAGCCACCAAACTCCTCTTCTCCTTTCTAGTTCTTCAGGAGGACGGTAGGTGTCCGGCAGGGGCTCCTC
GCTTCCCGAGACAGTCTTCACATATATGTGCCCTTCTTGCCTGCCCCGTGGGTTCATTTCATGTGTGGGTGACCGCCTAGGATTCTTGAGAACA
CAACAGATTTTTAAAATGATACAGTAGAGACTCCAGCTTCCCGCTAAGATGAACTCAAACAGCCATATTGACCCTTGAATGGGAACAGCAAAAT
TCAGACCAAACATTTGAAACAATGGTTGGCAAGACAGTGCGCATCGGGAGAGTAAAGACACTGAACCTTGAGGGGCAGGAAATGAGTGACAGTT
GCCTGAGCTTGTTGTCATCAAGGGGTTTTCAGGCCATGCAACTCTCCCTGGCTAATTATTGCTGGCAGACCGTCTCCAGCTTTTAACTTTTAAT
CTATTCGAGACTATATTCAGAAAGTGTCGTTCTATGGGTTTTTTTTTTTTATAAAGTTGGCTATTGCTTTTACAGCCAACCAGATTCTCTCTGT
CTTGTAAGTGAAGCTACTTATATCTAATATGATTGTTGATCTGGGTGGGCTCAAACCCACTCTCTTGCTTCTCTTTTTCACTTGTCCTGTCTCT
TCACTTCTTAGTGGTCTTCTCCTGCTTTGTCCCCTATAAGCTTTAGAACCTTACAATGTTTACTGAAAATGTGTTGCGATTTATATCACAGATA
TATTTATTTTAATTTAAGATTGTCTATGTCTATAGATGTTTTGCCTAAATGCACACCCCTCGTATCCCACGAGGCCAGAAGTCTGCAGATGACC
TAGAATTCGAATAATACGCTGTAGTGAGCTGTTATGAGGGCCTGGAAACTGAATCCAGCTCTTCAGACAAAGCACTTATGACTGCTCAGCCGTT
CTCTAGCGCCACAATTACACGATGATAGGGATGCTAAGAGTACCACGAATTTGTGGCGGCTGACAGCTTGATGCTGCTGAGTCCAGGACCACAG
ATATAACCATCATAACTTAGCTATTGTTGAATGTCTTCTACTAAGTTTTATTATTTCCATCATAGGATTAGACATTCGATCAGATTTATTCTTT
AGCATTCTATGTATTTTATTACCACTATAAAATAGGGTTTTTATAAAATTCCTTTTTCTAACTGATAGCAATTGATATTAAGAAATACATATTG
CTTTTGAATTTTTTTTCAGTGCTGGAAATCAAACATAGGCCTTTGCTCATACAAAGTGGGTACTATACCGCAAACCTAAGTCCTCTCTATTGTT
TTTATATACAATAAACATATTTGACTATTTTCCTAAGTCCAATAATTTACCTCTAGATTATTAAGTTTTTCTATGTTGACAGTCATATATCATC
TACAAATTATGATAATTTTGCTTCTTCCTTTTCAATGTTATATTTAATTTACATATACTTATATTTGTGAATATATATTTATATATATGCTATA
TTTGTTTATATTTCCTTTCAATGTTACATTTTTCTGTCCCATTGTGTATGTTAGGCCCTAAAGTTCAGTGCTGAATGTAAGTGATGAACACTCT
TGATTCACTTCTGATCTTCAAGTCATATTCCTTTGGGTGTCCCTTTTCAGGTTACCATCCTGTTCTAGCTTGGCTTTGAAAGTCCATGTCAAGG
GCTGGGCAGAAATGGTGCTTGCTAGCAAGTATAAGGATCTGAGTTTGAATCCCCAGCCCACATCAAAGCCCAGACAAACTACAGCCACCTGCAG
CCGCAGCCCCCGCCCCCGCCCCCGCCCCCGCCCCCGCCCCAGCCCCACCCCCAGCCCCGCCCCAGCCCCGCCCCAGCCCCGCCCCAGCCCCAGC
CCCGCCCCAGCCCCAGCCCCAGCCCCAGCCCCAGCCCCGCCCCAGCCCCAGCCCCAGCCCCAGCCCCAGCCCCAGCCCCAGCCCCAGCGCTCTC
TCAGGGAGTTGGGTGGCAGAGTCAGGAGAATCCCCGGAAGCTGGTGGGCCAGCACCAGCAGAGGAAAACGGCAAAGAGACCCTTCTTAAGCAAG
GTGGGTGGGAAGGGCTGACACTGAAGTTATCCTCTGACGTCCTCTCACATACATTCCATAGTATAAATACCCACACACAACCATGTGCACACAT
ACATTGTGCATACATTAAACAAACAAGTAAAACTGCTTTATCACAAGTGACTTATCTTTTAACAAAACCCCATTTTTTTCTTATTAAGATAGCC
ACATATCTGTTTGTTTTGACCATTAAATAAACTATATTAAGCTGATTTCCTTTTTCTGCTGTTGCTATATTTTCAGGACCGGGCACAGAACCCG
ATGACTTCTACATGTTAGGTCAGCACTTTACCACTAAGCTACATCCAACCCCCCATTTTCTAATGGTCAATCAGCTTTGAATTCCTGTCTGGTC
AGTAGACACATTGAACACCTCCATTGTGCTTCCACACATTTGGTTGACCTCCTTGCCAGAAGGTCTCACCTTTGCACAAGCGCACACTTAACCC
TTACCGTGCTACACTTTCAGTCACTCTCAGGGATCATAATCAAACGTGCTTACCTCAGATATTTTCGGATTGTCATAGAGTTCTGAAATTTCAC
ATGGTATCTCAGTCTCCTAAAATGGTTAAAAATAAATACATAAATAAATAATAAATAAGTGTATGTATGTATGTATGTATGTATGTATGTATGT
ATGTGAAGGGCCATAGCTCTAACCATTCAAATCCTTGTGTTTTAAGCCACTGTGCAGATCAGAATTGCCAGAAGCCTTTAAAACAAAGCAAACA
CATATATAAGAGCCCACCTTGAGCAAAATGAACAGATAGATTTAAGAAGCATCAGTCCTCGGCTGGCATCGGGAAGAAGTGAGCAAGAGCAGAC
ATGAAACCAGACTGGCCATGAGTTGATAACTGGGTCCTCTGTGCTCTACTTCCACAGTAATGAATGTGTAGGTACAGCAACCCCCCCCCCCCCC
GCTGCACTTGCAGTGGTTTCAAGTGCCAGCCAGGAATGGGGATCTTAAGATGTTAATGGAAATTTCAGAAACCAAAGGTTTTAAATGCCCTTTT
GAACAGCGTGACAAAGCCTTGAGCCACCCTACCTCATCAGAGCCTGGATTAGAAGCCTGGCCTTCTATCTCCCTATGTTAATATCTACCAATAG
AACAGGCATGGTTGTTCATCAATAGAGAATGAAATGACAACCCAGAAACCATTTGATACAGGTATGAAGCCTTGTCAGGTAGGATGCCAGGAGT
GTATACACATCCTTTTGTATGTAAGTCATGCTCACTTAGATACAGGAGTGGACAAGGCTATGGTCTTATCATATCCCTCCCATAGACACTTCAT
CCCTCTTTGTCTCTTGTCTTCCCTCATGTTCTTACCATCTGACTTCCCCCCTTTTCCTCCCTCAGTGTAGACGACACCCCACTGCACAGAGACA
TGTTTGCATGGCATCAGTCATCTCCCTCCATTCCTATCTTTTCCCACCCTGAATGTCATCTTATTGCCACAGATTTACTCAAGCAACCAGGGAG
TTATGTGAAAATCAGCTCTGCCGGAATCTACTGACAAAGGACATCACAGAACAGAAATATTTACTGTCTTGTCCTCAGAAACAACAGAGGTTCT
GAGTTCTGACATGCTAACGCCAGACAGAGGCCAGTGCACGGACGTACCCACTCAAGCTACTCATTCACACGCAAGAAGCCAGTTATCACCTCTA
CGGGCTCATCATCGATGGTTCTTCTTCCCTGGATCAAGGTAAACAGCAAAGCCACCAGCAGGGCTGTGGCCAGCAGGGATGGGATTCCGGCTGC
CAGTCCAATAGAGGGACCACAGTCCTGTCAGAGAGAAAACTGAGTCAGAAAAGCCCAGATTCTTCAAGAATACAGCTTACACCGCGTGTTCTAG
CACACACCTTTAATCCCAGCATTCAGGACGCAGACGCAACCAAATCTCTGTGAGTTTGAGGCTAGCCTGGTCTACAAAGCAAGTCCAGGACAAC
CAGGCCTGCTTACACTGAGAAACCCTGTCTCAACAAAACAAAACAAAATGTAGGACATATTCAGTGATTAAAATAAACAAACAAAAAAGGTGCA
TGGGCTCTGACTGCATTGGAAAAATGAAATATTTCAAAAGATATCTGTACTGTATATGACTGGTGACTTGCTCTCCTAGGACCTAACTATATAG
TTTAAGCCCATTTGCACTGCTGATCATGTGTTAAAACAAGGGACAACTGATTTGAATAGTAGCCTTACTCAACAGAAGCACAAACTACCCTTCA
GAAGAAGAAAAGCCAACGTACAGTTTGGAGGCACAGTTAAATGCCATAAATGCCATGTTCACAATCGCCTTCAGCCTGAAAAAGTCAAAGCTTT
TTTAAATGTTTATTTTTAATTTTTTTTTTTAGTACTGTGGAGAGTGGTTAATGTAGAGATTTACAACTAGCCAAAGTGCAGAGAACACATGTCG
GTGGAGTGCTCCGTTGTGACTGGGATTTCTATATTTTATCTCCTCTTCTGAGGCTCAAGGAATGTTTCAGAAGAGTGGGGAGAAAAGCTAGAAG
GGCCAGAAGTTGAGAGACACTAGGGAGAAATCAGTGTCTTACACTCAAGACTGGAGCATGCCACACATGAGCTCATGGCAACTGTGTTGTCTGC
ACAAGATCAAGCTACGCAACCGCCTAGCACAGCTCCGGCTGCGCACATGGGCTCCCACAGCTGGCTGCCGAGATGCTGCAGTTAAAGGCTTTCA
GCAGAGGGAGAGCCGGGTTCTCCAGAGGTTTGGTCCCTGGTAGGTCAGCCATACTCCAGTGGTTGACACCAAAACTATGCACATAAGTCAGCAC
GAATTGGATTTAACAGTTATTAAAAAAATTTAAAAGGAGAAGAAGTTTCGAGAGGTGTGGAGGGGAATTTAAGGAGAGCTGAAGTTGAGAATGG
GGTATGAATATATTCAAATATGTTGTGTACATGCACAAAACTCTAAAAGAACAATTAAAAAGTTATATTAAAAAGAATAACCTAGATAGAGAAA
CAAAAAGTCCGCGTTGTTAGCACATCCTATGCCATCTGTTGTTATTTTGAAAACATTCATATTGCATGCATGACCCTGCTTTCAAGCGCCCACT
CTGGTCTGAGTGGTAACAGACATCCTTGATAGCACCACAACGTTTCAGGGTTCCAGCATTTTCTGGAAACACTAAAGACGATGTGCAAATTCCT
CTAAACACCCTACAAATGCAACACTAGCGGGGTGTGATTTTTACAAAAGCGTGAAAACCCGTGTCACAAATACATGGAATGAGGACTGTTTGCA
GAATGTCATTTCACACATAAAACCAAACATTTCACACAAACAGACCAAGACGTAGAATGCTCTTGTAGAATTCTCTCCATCCCTTACTTGTGAC
CACCATCCCGAAGTCTATCCCTGTAGATGCTCCTTATTTGGTTTCCAACACACAGATCCAAACATAGCCATTGACTCTTCTATCCCTGGCTGCT
TTCCTCCATGCTCTGTGAATTTGCCTGAGTATGTGTGTGTGTCTTTCCTTGCTTGCATTTTATTTATTTATTCATTCATTTATTTATTCAACGT
GCATGTATGTGCACCTTGTGCATGCAGGAGCCTTCAGAGGTCACAAGACACCATAAGACTCTCAACAGCTAGG
MOUSE SEQUENCE - mRNA
ATTCTGGAGACACCACCTGATGGTACAGACAGAGCTAGACTGTCTGCTTCCATGGATCCACTGCAAGCAGTCCACCTGGGTCCTCGGAAGAAAA
GACCCAGGCAGCTGGGCACCCCAGTGGCTTCCACGCCTTATGACATCCGATTTCGAGACTTGGTCCTCTTCATTTTGGAAAAGAAGATGGGAAC
AACTCGAAGAGCCTTCCTCATGGAGCTGGCCCGAAGGAAAGGGTTCAGGGTGGAAAATGAGCTCAGTGATTCGCTCACCCACATTGTGGCAGAG
AACAACTCAGGGTCAGACGTCCTGGAGTGGCTCCAGCTGCAGAACATCAAAGCCAGCTCCGAGCTCGAACTCTTGGACATCTCCTGGCTCATCG
AATGCATGGGAGCTGGGAAGCCAGTGGAGATGATGGGGAGACATCAGCTTGTTGTGAATAGAAACTCCTCCCCGAGTCCTGTCCCAGGCTCCCA
GAATGTTCCAGCACCTGCTGTGAAGAAGATCTCCCAATATGCTTGCCACCGAAGAACCACATTAAACAATTACAACCAACTATTCACGGATGCC
CTTGATATCCTGGCTGAAAATGATGAGCTTAGAGAGAATGAAGGTTCTTGCCTGGCATTCATGGGAGCGTCCTCTGTACTGAAATCTCTGCCAT
TCCCCATCACCAGCATGAAAGACACAGAGGGGATTCCTTGCCTAGGGGACAAGGTGAAGAGTATCATAGAGGGAATTATTGAAGATGGAGAAAG
TTCTGAAGCTAAAGCTGTGTTAAATGATGAGCGATATAAATCCTTCAAACTCTTTACCTCTGTGTTTCCTGTGGGACTGAACACACCTCAGAAA
TGGTTCAGGATGGGTTTCAGAACTCTCACCAAAATACAATCAGACAAAAGCCTGAGGTTCACAAAAATGCAGAAAGCAGGATTCCTCTACTACG
AAGACCTCGTTAGCTGTGTGAACAGGCCAGAAGCCCAGGCTGTCAGCATGCTAGTTAAGGAGGCGGTTGTGACATTTCTTCCAGATGCCTTGGT
CACCATGACTGGGGGGTTCCGCAGGGGTAAGATGACTGGACATGATGTAGACTTTCTAATTACCAGCCCAGAAGCCACAGAGGATGAAGAGCAG
CAGCTCTTGCATAAAGTGACACACTTTTGGAAGCAGCAGGGGTTGCTTTTGTACTGCGACATCTTAGAGTCAACCTTTGAAAAGTTCAAGCAGC
CCAGCAGGAAGGTGGATGCTCTCGACCATTTCCAGAAATGCTTCCTGATTCTAAAACTGGACCACGGGAGAGTGCACAGCGAGAAGAGCGGCCA
GCAGGAAGGAAAGGGCTGGAAGGCCATCCGTGTAGATCTGGTCATGTGCCCCTATGAGTGCGCCTGTGCCCTGCTTGGATGGACCGGCTCCAGA
CAGTTTGAGAGAGACCTTCGGCGCTATGCCACACACGAGAGGAAGATGATGCTCGACAACCACGCCCTGTATGACAGGACTAAGGGGAAGACTG
TCACGATTTCACCTTTGGATGGGAAAGTGTCAAAACTACAGAAAGCCTTGAGGGTGTTTCTTGAAGCAGAAAGTGAAGAAGAGATCTTTGCCCA
TCTGGGACTGGACTACATTGAACCATGGGAAAGAAATGCCTAAGACGGAGTTGTTGACATTTTTCTTTTCGGTTTAGACAAGCTGTGCTCTGTG
TTAGTAAAAGATGGCTTGGGAAAGTCTGGGATTCTTTGACCTCAGAGGATGCTGGCAGCAAGTGGATCATAAGTATACTATGCAGAGAGAATAT
TCTAAGGCAACTTTGAAGTCTTGATAAACCAGGATGCAGCCATGGTTCACAGGTCTTGGGCCATCTGGCCTACTGTTGCATAGAATTCATAGTC
TTTTGTCTGCTTTGTTTGAATAAAAATGGTGTTGTCATTGCAGCATCATCTAGAGAAGCGCATCAATGCCCACCTGACTCCTGTGTGTCTGAAT
CTCTGTGTATTGCCAATAAAAATAGGAAAC
MOUSE SEQUENCE - CODING
ATGGATCCACTGCAAGCAGTCCACCTGGGTCCTCGGAAGAAAAGACCCAGGCAGCTGGGCACCCCAGTGGCTTCCACGCCTTATGACATCCCAT
TTCCAGACTTGGTCCTCTTCATTTTGGAAAAGAAGATGCGAACAACTCGAAGAGCCTTCCTCATGGAGCTGGCCCGAAGGAAAGGGTTCAGGGT
GGAAAATGAGCTCAGTGATTCGGTCACCCACATTGTGGCAGAGAACAACTCAGGGTCAGACGTCCTGGAGTGGCTCCACCTGCAGAACATCAAA
GCCAGCTCCGAGCTCGAACTCTTGGACATCTCCTGGCTCATCGAATGCATGGGAGCTGGGAAGCCAGTGGAGATGATGGGGAGACATCAGCTTG
TTGTGAATAGAAACTCCTCCCCGAGTCCTGTCCCAGGCTCCCAGAATGTTCCAGCACCTGCTGTGAAGAAGATCTCCCAATATGCTTGCCAGCG
AAGAACCACATTAAACAATTACAACCAACTATTCACGGATGCCCTTGATATCCTGGCTGAAAATGATGAGCTTAGAGAGAATGAAGGTTCTTGC
CTGGCATTCATGGGAGCGTCCTCTGTACTGAAATCTCTGCCATTCCCCATCACCAGCATGAAAGACACACAGGGGATTCCTTGCCTAGGGGACA
AGGTGAAGAGTATCATAGAGGGAATTATTGAAGATGGAGAAAGTTCTGAAGCTAAAGCTGTGTTAAATGATGAGCGATATAAATCCTTCAAACT
CTTTACCTCTGTGTTTGGTGTGGGACTGAAGACAGCTGAGAAATGGTTCAGGATGGGTTTCAGAACTCTCAGCAAAATACAATCAGACAAAAGC
CTGAGGTTCACAAAAATGCAGAAAGCAGGATTCCTCTACTACGAAGACCTCGTTAGCTGTGTGAACAGGCCAGAAGCCCAGGCTGTCAGCATGC
TAGTTAAGCAGGCGGTTGTGACATTTCTTCCAGATGCCTTCCTCACCATGACTGGGGGGTTCCGCAGGGGTAAGATGACTGGACATGATGTAGA
CTTTCTAATTACCAGCCCAGAAGCCACAGAGCATGAAGAGCAGCAGCTCTTGCATAAAGTGACACACTTTTGGAAGCAGCAGGGCTTCCTTTTG
TACTGCGACATCTTAGAGTCAACCTTTGAAAAGTTCAAGCAGCCCAGCAGGAAGGTGGATGCTCTCGACCATTTCCAGAAATGCTTCCTGATTC
TAAAACTGGACCACGGGAGAGTGCACAGCGAGAAGAGCGGCCAGCAGGAAGGAAAGGGCTGGAAGGCCATCCGTGTAGATCTGGTCATGTGCCC
CTATGAGTGCGCCTGTGCCCTGCTTGGATGGACCGGCTCCAGACAGTTTGAGAGAGACCTTCGGCGCTATGCCACACACGAGAGGAAGATGATG
CTGGACAACCACGCCCTGTATGACAGGACTAAGGGGAAGACTGTCACGATTTCACCTTTGGATGGGAAAGTGTCAAAACTACAGAAAGCCTTGA
GGGTGTTTCTTGAAGCAGAAAGTGAAGAAGAGATCTTTGCCCATCTGGGACTGGACTACATTGAACCATGGGAAAGAAATGCCTAA
HUMAN SEQUENCE - GENOMIC
TTTGGGATTCAGATTCTATTAGTGTGCAATGGAGCCTGTTCACTGATATTTTAAAAACTCAGGTAGTTTATTGTGTAGCCAGGGTTGTGAACCA
AAGTTCTACACAGAGAACAAATATTTATGTAACAATGTATTCATGTAACAGGCAAACTGGGTTTTTGTTTGTTTGTGTTTGCTTTGTATCCATT
GCTCTGGCCTGAATGTTTGTTTCATCCACAAATTCACATGTTTTTGCCTTAATCCCCCATTTGAGATTAGGTGTATTTGGAGTTATACCTTTGG
GAGTAATTAGGACATGAGTGTGGAGCCCTCCTGAACGGGATTAGTGCCCTTAAAGGAGGAAACACGAGAGAGATGATCTCGTTCCCCACCATGT
GAGGATACAGCAAGAAGGTGGCCATCTGCAAACCAGAGCTAGTGCCCTCACCAGGAAACTTTTCAGCTGGCACCTTGATGTTGGATTTCCCAGA
CTCTAGAATGGAGAGAAATAAACTTCTCTGGTAATTTTGTTACAGCATCCTGAAGTGACTAAGACACCCTGGTGATCTGGGAGACTCCAATGTG
TGGAATTTACCTAGTTTTCCTCTAAGACCTGCCAGCCTCCTCTTGGATTTTCCTCGCACTCTTCCCCTTCTCTCCCTGTGCCCTCCTCCTAAGT
TATCTAAGGCAAACCTCTTCCTCCAGTAGAAGTTCTTTCTGTTGGACACTCTTCTGCCCTTTCTAATAGAGCTGAGATTTACTAGTAGTACATA
GGTTTACTTCCGCTGAGAATAAGTAATCTGCTCTCTCAGAAGCTTTGGAAAGGACCTAGGCCCTTTTTCACCAAGTTAAAACATTTCAGTTAAA
TATAATCACACCATTTAGCACCTAATTATAAACTATTACCTCCACAATTTAAAAATATTGAATATGAGATTTTGTTATTTTTAAAAAGTAAACA
ATTACTCACAGAGCTTTATAAAAAAAGATTACTATGTTGAAAGTGAGTCCCTACTTCCCCCTACCCCCATCAAATTATTCTCTCTGAGAAACAC
ACGTTTAAAATAAATCTCAAATTCGTTGCATTGAGAGCTGACTTTGGTACCAAATTCACGATGTGACATTGTTTCTGCTGCGCCGTTGAACCCC
TTTTTCCTCCCAGGTGGTGATGAAAGACTTTCACTTCCTGGCAAAGAATAAGAGAGGGTTTTGGCTGCATCCCCAGCAAAACCAGCTTTAGATA
AATGGTTTGGCACAAACACTTGTGGGCCTGAGCTCTCCCCTTCTATGCATTAGGGGTGTGTCACTGACAGAATTCTCACTGGTCTCTCTGGATG
GAAGAGGAATGGTGTTCACAGCCTATACTTTGGGCTGTCATCTATGTACAAAGACATGTGCTAAGCCCTCTAACACATACAAAGAAGGTTACAC
TCTAAATGGGAAGACGAGCCATACTTGTCTACATAAAGGAAGGTGGTCAGGGGCAGAAGGTAATTGAAACAGGGTAAGTATCAACAATGTGTTA
TTAAGGTGAGTTCCCAGGAAGCCAGCCCTGAGATGGGAATTTGTGTGCAGAAGTCTATTGGGGATGCCCTTGGGATCATCATCTATCGGAGCAT
GAAGGAGGAGATTGGCCAGAGAAAGAGTTGAACCACTATTTGATTGTAGTACAGATCTCAGCCAATCTAATAGGATGCTCTAGAGTTACCCTGG
CCCTTCAGAGTTGTCACCCCTTGAAATGAGATGACCTGACCTTTGTAACCTTCCCCCGATCATTGACCAATAAGTGGAGGGGTCTGTCCTTAGG
CAAGTGTCTCTCTTTGTCAGTGAGCCATCAGCTCTGCCATTCTCAGTAGCAAAAGGAATGGTGACTCCATCCTCAAGGGGTCTCTGGGCAGCTC
ACCACAGCATCCACTACAGAGGGAGATCTGTGACCTTGAAGATAAATGTGTTTTTGGTTGTTGCTGTTTTGCTTTTTTTTCTTTGAGATGGAGT
CTTGCTCTGTTGCTCAGGCTGGAGTACAGCAGTGTGATCTCGGCTCAGTGCAAGGTCTGCCTCCCAGGTTCAAGTGATTCTCCTGCCTCAGCCT
CCTGACTAGCTGAGATTACAGGTGTGTGCCACTATGCTCAGCTAATTTTTGTATTTTTAGTAGAGACAGGGTTTCACCATGTAGGTCAGGCTGG
TCTCAAACTCCTGACCTCAGTTGATCCTCCTGCCTCGGCCTCCCAAAGTGCTGGGATTACAGGTGTGAGCCACTGTGCCTGGGCCAAGCATAAA
TGTTTATAGCACGAGGGAAAAACGTACATTAGTAAGGCACTGGGAGTAACTGGGGAATCTGAAGCGTATTGTTATGATCAGTCTCTACTTGCTA
TTCATTTCACTCTGATAAGACCATCTCAATCCCCTAGTGTTATAAACACTACAAATACTACTATAGCATAAAATGTCCTCCATAAGATCCAGTA
AAGAATAGCTGAAATATTTAGCTGACTCTTTTCAAACCATAAAGGTCAAACACATTTCAAGTTTAGTTTAATTCTATTTTACAGTTTTGGTTTT
ATAACCCAAAACTATTGTTTTCCAATTCACTGTCTAAATAATTGTCAGATTCTGTCTGTCTCTCTCTTTATTTATTTATTTATGAAATCAGATC
TGGCTCTCTTGCCCAGGCTGGAGTGCAATGGCACAGTCATGGCTCATTATAGCCTCAACCTTCTGGGTTCAAGCAATCCTCCTGCTTCAGCCTT
CTGAGTACCTGCGAAGACAGGCGTGCACCACCACACTCAACTAATTTTTTTACTTTTATTTTTGTAGAGATCGAGTCCTGTTTTGTTGCCCAGG
CTGGTCTCAAACTCCTCGTCTCAAATGATCCTCCTACCTCAGCCTCTCAAAATGCTGGGATTATAGGCATGAGCCTGGCCTCAATTTTTTTTTA
AGGTAGTCTTTTTTTTTTCCAGTAGTTTTTGGGGAACAAGTGGTGTTTGGTTACATGGATAAGTTGTTTAGGTGTGATTTCTGAGACTTTGATG
TACCCATCCTCTCTCCTTTTACAATGATTTCTTTATGATGTGCCTCCTTGGTCTGGCACATTTATGCCTGTTTCCATCGCTACCATCATTAGTT
TCTTTCAGATCTTTGCTAAAATGTCACCTCCTCAATAAAGCCTAATTGGATAATCTTAGACTGCCATCCCAACCCTGGCATACGCTGTCCCCTT
TATTTCTCTCTACAGCATTTATCACCATCTGGCCTGCTTGAGATTCTTGAATAATTTTATTTATTGTCTGTTTCCTTCTAGTAACAGACAAGTA
TGTAAATCCCATCACGCAGGGGTTGTTTTATTCACTCTCTATTATTAATGCCTAGAAGTGCCTCGCACATAGTAGGCACACAGTCAATATGCAT
TGAATGAATGAATGGTCCCAGCTGAGGCTTCCACGGTTCTCTTCTGGCCATTTGCAATAGTGACCCAGCTGGAGTAGCAGCTTCAGCTCTCTGC
ACCTTCCAGGAATCCCGTTTTGGACCACGCGATATACACATCACTGCAGGGCTGCCTTCTGTGGCCCTGCCTCGGGCCTGCCACTGTCCTGCTC
AGAAGCTCCCATGACTTCTTAGTGCCAACTCAGGCTCTGGGTGGTCTCTGAGGTCTGCTCCAAACCTAACTTCTCTCCTTTTCTCCCATCTGCT
CAAATCCTTCCAGCCCTCCTGGCCCCCGACAGGCTCCCTCTCATGTGAATGCCCTTCCTGCCACCCTTTCCTGCCCTCCTCTGAATTCCTCCAG
CAGATGAGTGTAAGGGGGCAAGTTAAACATCTTCCAGGGCCTTCTGAAGAATGTGAGGATAAATGGTAAAGCATTTCACAAAGCCTAACATCTT
TAGAGATGACTATTATTGCCAGTATGTCATGATAGTATTATTGTATCATAAACTTCTCAATTCAGTAATAGATGTCTGGAAGGTTGAAGCTAAA
GCATCAAGCCACATTCTCCAGTTTACCAGTAATAAAGCTGATCTTGTGACTCTGGTCACAGTGGTGTGCTGGAGTCAGCTGCTGCCAGCTCACA
AGAACCCATGGCTAAATCTTCTGGAACTCTGCGAGCCGACTGCTAAACATTCACATGCACCCCACTGCCAACTGACCCATGCTTGTTTTGCAGT
CACTTCATAAGCTGCAGGAGAGAAAGATGTCTTTAACTGCTTACCTTAGAGACGTTATCAGTCAACAAAAAGAATTAAAAGGATCTGGTGACAG
AAGTGAAGGAACAGTTTGGGGAGTGATGATCAGTAGTTCTGTTTCAGGCATTTTGAATAATATTTTTCAGTGGAATGTAAACGGGCCTGGTGCT
GAAGAGAGAGAAATGGGCTAGAGATAAAGATTTGGAAGTTATCGGATATTGCCAGGACTGGAGGATTTTACCCCCGGATACCCAGGAGCCATGG
CCCCAACCACTGATCCACAGCACAACGTCTATCATCTCACTCAGGCTCTGTTCTCATTCTCTCCAGTGGCCCCAAGTCATATCTTATTATAATT
GTCTCCTGATTCCTTCCCTTCTTTCTCCCTCCTCAATTCACCCCATTCATTATTGCCTGATTAATTTCCCTAAGCCACCATTTTGATCACCTTC
TGTGTTTGCTTAAACACCTTCAGCAGCTCCTAACTCTTTGCAAATAAAATCCCAACTGTCCTATTCAAATTTGTCCTTGAGGCAAATCCCTGTA
CTTTCTCCCCTCCATGCCTGATATTCATGCTGTCACCCATAATTTTGTATACAGTTAAGTTTAATAAACATTTTGGAATAAAGACTAGAAGTCA
CAAATAAGCTCCTGTTGTCCATCAGTTCCATTGTTCTAACTATCACTTCTCATGTTGTAGATGAAGGTGAACCCCTGTTATAACTAAAATATAG
AAATTTCTTGTACTTGTTAGGCACCCAACATATATAGCTACCAAATGCTTCTTAATGGCAAGAAACAAATTTTCCAAGGTTTATTTTTCATTCA
AGACACTCTACACATGCTCCTGGCAAGGCAGTTCTAAGCTCCAGAAGACACTTGCTGCCAAGAACCAAACACAGCTGTTCAGGCAACAGAGGAA
GCTGGTGCTCGGTGAAAAACCAAACCAAAACAAAACCCTGCCCGCACATTTGAGGATGGCACAGGTCCCAGATCTACACCTTGAAACGAATAGA
AGATACAAGATATTTGATATAAAAGAAAATGAAAGTGCCGGAAACAGAAGTTTTCTCTCTCAAACTGACTTCCTCCAACGTGCACAGAGAATAA
CAACAGGACTCCTAAGCATAAAAATAGTGTTTGCTGATCGGAAGAGGCCTACTTCTCCACTGAACCATGGAAAACCTAAAATCCTCCCCATTGT
TTCCTGCCCCATACACTTGTACTCAATAAAGATCTCTAAGAATGATCATGTGGAGACTAAATATTACCCAAAATGGAAAAAATTTAAAAACAAT
CCCGAACTAAGGGGAGAAGTGGCTGCCTGGGAGCATCAGCATGGAAACCAGTGAGCAGCTGTGTTCTTTGTTTCCTCATAGCCATCATCACCTG
AACAGGCAGGAGCATTCGTCACATCTGCCAGCATCATGGGAGTGGCAGCATGAAAGGGAGGACAGAACAGTGGCCCAGCTCCCATGTGGACCAG
GCAGGCAGTTCTCATGCTATCTTATTCATCACTTACAATGTGAAGAAGTTGGCACTATGACTGCTATTTTACATGTTGGAAACTGCGAACCCCC
CATGGGGTAAGGGAATGGCCCAAGCTCATACATCTCGTAAGTAGCAGGTGGTTCAGGCTTGTGTAGCTCCTGAGGCACCAATACTTCCAGTTGC
ATTCAAAAGGGTGCTGTAGGTGTTCTACAAAACAAACAAAGAAAAGGTAAGCATGTTGAGAATACAGTGGGCGAACACAGTTTATTACAAACCT
TTAATGTTCTAACACACATTGTATAGAGTTTCTCTGGTTTCCCTACACTTAATTCACTGCAGAATTTTTGTTTTGCCTGGAACACTTCATGCGA
CCAGTGTTCCCATAAATCATTGCAGGACACCCCTCTGGTCTCATTAGAGTCTTAGGTCTGTTTTATTTTTTTGTCTTGTTTTGTTTTCTTTTTT
GAGACAGGGTCTCACTCTGTTACCCAGGCTGGAGAGCAGTGGCATGGACAGGGCTCTTTGCAGCCCCGATCTACTGGGCTCAAGTGATCCTCCT
GTGTCAGCTCTTCCTAAGCGGGCTGGCACCACTTTTGGAAGAGTCTAGGGAAAGCAAAAGACCATTGGGAAGGATTCTGACAGGATTACCTACA
AAGTATATCCGAACACAGGGAAGGGAAGCCCCAACAAGGAGAAACCTGCACTATTACAATAGCAATGGACAGTTCTTGGAACTTGCATGCCAGT
GGTCTTTGAAGTGCTTTAGTCCTCATAGCCACCTGTAAGATAGAAATCATTCTAACCCCCATTGCAGAGATAAGGGAAATAAAGCAAAGAAAGG
TTAGGTTTTGAATTCAGGTAGTCTGGCTCCTAACACCTGTATTCTATTTGTTCATATTGAAGAGTCTTAAATGGAGCAGAATTCTCCACCAAGT
AGCATAAACAAATAAACTTTTGTTAAGACACTGAGATTTGGAGATTATTGACCAATCTAGTAGATCAAGGAGAGTGCTCAACATCTGATGCAAA
GAGCTTCACAAAATCATAGTATAGTAAGAAGAAATCAATTCCTGCTATTGGCAGCAATGCCAAAGAGCATAACACAACTCTGTATAGTGTTTTA
TTATTTTCAAGGCACTTCATGAGCACGATTTAATTTAGTTCCTGAGAAGGCGATGAGACATCTGTGCTCTCACACCTTATTTCACACCTCCCTT
CACCACAGGGGATTCGTTTAACAGACATTAATTGCTGAAAAAGCAAAAAGACCACATTGAGGTAACTCAGAGAAACAAACTGCAAGAAGCACAC
TACACCACTGGGCTGGAGCAACAAAGGGATGGAGCTGGGATTGTTGAAAACTGGAAGTTTGCAGCAGGGCTCCCATGGAGCTGGAACACAGATC
TTTAATAAGGAAGTGCTGCCTGTCGTCCCTGGTGCCATAGAATTTCATAGGGAAACCTCTGCAGAGCTGGGGCTCAGACTTTTGAGGAGATGTC
ACCTAAGTGGTATTGATACCTCTGAAGGGTACAGTAAGACTGGGTCTGGTTGTGCAGAAAAGAAAAAAAGAAAAAGAAAAAAGCTAAAGACTAG
AACCAATCACTCCTGTCTGAGTAGAGAACCATTACTGCGAAGACCCTGACCTTTGTAGCAGCAAGCAAACAAAAAAGGTCACCTCTTTCCTCTT
ATCTAGACTTTCAGTCTCCCTCTGGTATCACCATTGGCAGAGCCTGACATAAAGTCAGCTGGCAAATGAGAAAGAGAATTTGCTCAGTCCCAGC
CTCAACCTCATGGAATCCATCAGAAAAAAGGTAGATTTGCAGCTCAGAGATGATAGCCTGATAACCAGACAGGCATCAGTACATTTGAAATTTT
TGTTGGTAATGTTTTCAATACCAGTGACTCAAAGCAAATTAAAATGTGACATTGTTTAATATGATATTTTGATCATTTAGTAGTTTCACAAAAC
ATATATATCCGATTTCAGATTGAACTCGAATGTCTCTAAAAACAAAACATAAATTACTACGAACTGACATTTATTGAAAGTGCTACCTTTGTGT
CAAGCACTCTGCCCAGCAGGTTATGTGGGCTATCTCAGTCCTCATTTTACAGATGGGGTAAACCTGAGGCTTAAAGAAGTTAAGCAACTTGCTT
TCAAGCTTCAGAGCAATAGAGACAGAATTTTTATGGGCAGCCTGACGAAGCCTGAGCTGTTAGCCACATCTCAGTACTACCTCCCCATTCCCTT
TACTGCTTACCTGGTGCACCTTTACTATTAGGTGGCTATACCTTGGGAACGAACACAACAATCCCAGACTCTTGCTAGAATCATTTACTTAACT
CCCTGCTGCAGCACCATTTGGTAGCTAAGACTGCATTCTAACATGAAGCATTCAGCTCTTCAGTAACCAAACACATAATAGTGAGGGTCAAAAT
AGACTCTCATCTTCCAGTTGATCTCTAGCAAACAGTCATAGCATCCGGGAATTTCAGGGCTGCATTATAGGCCCTTACCAGATGTCTGAATTCC
CTTCTGTTACAGCTCAATAAACAGTTATCCAGCATCTGTTTGAATACTTTTCATGACTTGTAACACACAACTGTATAAAGCTGCTCTCTGTATA
TTTTTAGACAGCTGAGTTACTAAACACGTCTTACTTGCGGAGAACTGGAATCTATTACTCTACAATTTCCACCTGAAGATTTCTGATTTTGCTT
TCTTGAGCAACACAGAGTGCTTTACCCATGCCTTAAGGCAGCTGTCACATGACTCTTCCCCTAATACGGTTGTGAGCTGCTCACGTGTTTTGGA
GATAGCAAAAGTCTCAAATAATATGGCACAGACGGCCAGCCATGGTGGCTCACACCTGTAATCCCAGCACTCTGAGAGGCCGAGGTGGGCCGAT
CACCTGAGGTCAGGAGTTCAAGACCAGCCTGACCAACATGCTGAAACCCCATCTCTACTAAAAATACAAAAATTAGCCAGGCGTGGTGGCGTGC
GCCTGTAATCCCAGCTATTCAGGAGGCTGAGGTAGCAGGATCGCTTGAACCCAGGAGGTGGAGGTTGCAGTGAGCTGAGATTGTGTCACTGCAC
TCTAGCCTGGGCAATAAGAGCGAAACTCCATCTCCAAAAAAAAAAAAAAGGACAAAGGGTGAGCAGAGCTCAGATTGGTTATTGGAGAAGACAA
TACAGAGGAAGGCTCCTTGGTGGTTAAGAGTAGTCTTTTAAAATCAGACAGATCTGAGTCCAAATCCTAGCTCAATCATTTCTCACTTGTGAAA
ATTCAGTAGCTCTGTCACCTTGGGCAAATTGTTTAGCCAAATGAGAAAGGTAGGACCACCTGACAGTGGTATTGTGAGGATCCTGTCAGCTAAG
GCTGGTAGGTGCTTGCCTTGGCCGTCTACTAGCTGCTCAGGAAATGAGATCTATAGAGTCTATTGGTGAAAGACAAATTTAATTTTGCACATAA
AGTTATGAAAATGTCTCCTTATGGCAGTGGTTATCATCTAAAACTCATACCATCCCACTGAAGAAGACCTATTTTAGGGTTTATAACAGACTAC
TCTCTGGACAACCTTAATCCACTTAAAAAACTAATATATGCATACAGCATTTGCAATCCAAAGAACTGAATTCAAGATATGGCTTCGCCCCTTA
TTCTGTCATCTTGAATAACTGAGCCTTGGTTCTCTTGCTTATAAAATCTACCTATGTAAGTGAACAGGTGGTAATACCCACCATGAAATACTAT
GGAGCAGTTAGAAGCAACAGAGCAATTACAATACGGATCTGGAAAACATAGTTCCAAGTGAAATAAAAAGGTGATTGGCAAAATGAGAAATGTA
ACAATCCCATTTATGTAAAGTCAAAACAACCACAAGCAGAATATAACACCCATGCAAATAACAGACATCCAGTACAGTGTAATGATTGTCTACA
AAAGGAGGAGAATGCAAGTGAGTTGGGAGGTTTAAAGGGGATCAATAAATAAAGACCACAGAAGGGCCTCAGTACATTTAGAAATAAATAACAT
GCAAACAATGATGCTTCCCTACCTTCCTCACGAGGTTACTCTGAGGAGCTAATGAGATTGTGCGTATGAAAACAAAAGTTAATTGACTGTCTTC
ATTATGATCTCCATATTGGACAAATGAACTGAACACAGATAAAAATTCCCCATCACACTTTGGCAGGAAGCTCTTGCCAGGGCAGCACCTGTGA
AGCCCTGGCCTGGCTTCAGAGTCTGCTGGTGAGATGACATCAAAACCCTTCGTGTAGGAGGGTGGCAGTCTCCCTCCCTTCTGGAGACACCACC
AGATGGGCCAGCCAGAGGCAGCAGCAGCCTCTTCCCATGGATCCACCACGAGCGTCCCACTTGAGCCCTCGGAAGAAGAGACCCCGGCAGACGG
GTGCCTTGATGGCCTCCTCTCCTCAAGACATCAAATTTCAAGATTTGGTCGTCTTCATTTTCGAGAAGAAAATGGGAACCACCCGCAGAGCGTT
CCTCATGGAGCTGGCCCGCAGGAAAGGGTTCAGGGTTGAAAATGAGCTCAGGTAGGACAGCATCGATCTTGCTTTGTAAATAAGCAGAGGCTTT
GTGAACAGCTTCTTGGGAACCCAAGGAACCTGTGTTTTCTTCCACATGTGGAAGAGGTGATCTTCCCACTGGGAGAACAGCACATTTGAAATCA
AAGAACAACTAGTTAAGGAAAACGAGATAAGTGGGGACTGAATACAATTATATTGAAACTAGTTGACACCAAAAGATGAGATAAATTTTTTGTT
TTTTGCTTTTCACTCCTATGAGATGTGTTATTTAGGGATGATAAGCAGAACACTAATTGGAAAATTTAATCCTGAACCACTCAATTCTATTCTT
GACCAATTTGAACAAAGTATCACTGGGTTCAACAAAAAAGTTTTGCCTACTTTGATGCAGAAAGATGATTTGGTTCCTTGGTTCTTACCTATAC
ATCTGGGCGTGCTAAGGCAGCTAGCATCTGTGGTGGGCGAGGATGACTAGCCAGCTTACCACAGCCACCCAGATGTGGCCTTGACCACATTTTT
CCAAACAGTGGTCCTAGTGGGGTTCTTAGAAAGAGAGATGCTAGGTAAAGATGTATTCACTGTTAAGACAGCTCTGTCCAGGGATGACAGACAT
CACACTGACTGAGATGTAATCAGACCCCAGGCAAGGAGGCGATAGAAGCATTTGGACCACCTAGATTCGTTCACTCACTAATCCACTCACCTTG
AACAGCAATTATTCTTGTTGTTTTTCTAACTTCTGTGTAAGCTCTTCACTGATCTCAAATACATCAAGAGAAAACAAGCAAAATCCAGAAACAA
ATGGTATTAGAACCTTGACATAAGTGATGGTTCTGGATTAAACTGTTAAGGGGCTTGCAATGAAGGAGTAATGATTTATGCAGTTGACCGCTTT
GACTGTGCAAGAAATAAATTGACCACAAATTTTCAGCCAAAACTATAAAAATTTTATGTGAAAGGACATTTTGATTGTATAAATAGTCTTTGAT
ATTTGAAAATAAAACTGACAAGCATGTCATGCACAAGAAATGTCAGCTTCCAAGTGTTGAGGAGACATATTTCAAAAGTCAGTTTTCAGATATG
TTAATGTTCGCAACACCCTGATTCATATGACATTGACTTTTCATTCATTTTTTGCTAAAATGCCCCATTCAGAAATTCCTGAATTTCCTATGGC
CCTTCTTCCACCTGATTTCTCTTTGCAAGGTCTCCTTTTCTCTCTTGCTGCCTCTTTGTAACCTCCTACAGCCACTCTGAATCTCATTTTCTAA
CATCCTTTCCAAGAAAATCCTAGATTTCATCTAACTGTTAAGCCTGCCTGATATTACAGACTTACATAGAAGGCTAGACTTCGAGAAGACAGGA
AGATTATATAGGTAAAGAAGCCAAAAAAATAGATTTTTTTTTTTTTTTTTTTGAGACAGACTCTTGCTCTGTTGCCCGGGTTGGAGTGCAGTGG
CACAATCTCGGCTCACTGCAACCTCCGCCTCCCAGGTGCAAGTGATTCTCCTGCCTCAGCCTCCCAAGTAGCTGGGACTACGGACACGCGCCAC
CACAATCACCTAATTTTTGTGTTTTTAGTAGAGACGGGGTTTCACTATCTTCACCAGGATGGTCTCTATCTCCTGACTTTGTAATCCGCCTGCC
TTGGCCTCCCAAAGTGCTGGGATTACAGGCATGAGCCACCACGTCTGGCCAAAAATAGATTTTTTGGAAAAGAAATTAAAACCCTTAGCAAATA
TGGAACTGCTTTTCATGGTAAAGAAATTTGACAAGGGTTAGGCAGGAAGGAAAGAACCAGAAGCACCACTTACTGGAAAGCTCTCCATTGAAAC
GTCCAAGCTTCCAGCAAGGACCCGCTGATTTATTATAGACACAGCAATCAATACATACAACGCGGCTGAGGCAGGCACTCAGCGAGATGATGGG
AGGTAAGTTTCTTCTGTTCGCAGGACACCTGAAGAGGCAGCTACATGCCCCAGATGCCTGCTGCCAGGGACCAAGCCCAGCTGCTCAGGCGGCG
GAGGAAGCTCACTGCTGCATGGTGAGCAAACTGCCGCTGTGCATGTGATGATGGCAAAGTTCCCAGGCTCGTAGCTCAGAAGACACATTAAATA
CAAATGTGCTCTATGGCGCAAAAATAAACCACAGGAAGTAGAAGTCCTAGGTCTCAAACGGAATTTCTGTGCATAGATATATAGACTTTAATTG
GATAGAGAAATAAAAATTATTGTAGGGGACAAATCTCTAGATACGAAATTTAGAAACCTCAAATCCTGGCTCTGAGCCTCCCCAGCCATGGGGT
ATGGGACAAGGTCCCCTCTCTCCCGACTGATTTTAATATCCTGATAAATGGCCTATCTGATGTGATCACCAAATGAGATAATGCATGTCAATGT
GCTTTGATAAATTAAAACAGTTAGACAAATGTAAAGTGTAATTATTTTCTTGTGAATTGCTGGATCCATTGACCTTTACAGCAAAACCCTAAGG
AATGGGCTCTCCCTCCACAATATGAACAAGGTCAAATGATATCAACAAATAGAACAAAGACAGGAAAGAAACCCGTCAACATGTTATAACTGGT
TTTTTGGGAGTCGCGGTTGGTAGGGAGAGATTATGGCTGTTTTTATTTTCTGGGTTTTCCAGTTTTTTTTTTTTTTTTTTTTTTTTTTTTTTTC
AGACGGAGTCTCGCTCTGTCCCAGCCTCGAGTGCAGTGGCACGATCTCGGCTCACTGCAACCTCCACCTCCCAGGTTCAAGCGATACTCCTGCC
TAAGCCTCCTGAACAGCTGGGACTACAGGCACGCGTCAGCACGCCCAGATAATCTTTTTTATTTTTAATAGAGTTGGGGTTTCACGATGTTGGC
CAGGATGGTCTCGATCTCTTGACCTCGTGATCCGCCCGCCTTGGCCTCCCAAAATGCTGGGATTACAAGCGTGAGCACCGTGCCTGGCCTCCAA
TATATTTTTTACAATAAGCATATATATATGTATGTGTCTGTGTGTGTGTATATATATATATATATATATATATATAAGCATATATATGTGTGTG
TGTGTGCATATATATATATATATATATTTTTTTTTTTTGAGGCAGGGTCTTGTTCTGTCACGCAGGCTGGAGTGCAGTGGTGCCATCTCGGCTC
ACTGCAACCTCCGTCTCCCAGACTCAACGGATCCTCTCACCTCAGCCTCCCAAGTAGCTGGGACTACACATGCCGGCCATCCCCGGCTAATTTT
TGTAGAGATGGCGTTCCGCCATGTTGCCCAGGCTGGTTTTGGACTCCTAGCTCCTGCCTTCACCTCCCAAAGCATTACACCTCTGAGCCACCGT
GCCCAGGCTACAATAAGCATGTACTTTAAAAATAAAAATAACAAAGAAAGCTGTTTTTTCAGCTCATAGGCAACAGTTTTCATTGTTCCTAACA
CATTCTTTGGCTGTCAGAAGGATTAAATTAGAGAAGAAACCTGATTCCTGCTTTACTTGGGCCTGAACACACAAATTAGAAATGGTGTACCTCA
AAGTGTTGCACTGTCTGGAGCTATTTCATTCTGCAACATGCATGCAACATAATTGGGTGGAGCTTTGCTAACATTTATGCTATATTGCAAAATA
CGTGCCATTCAATGTGCCATAGGAAGCTGGTCTTAAAACGTAGATAAGGGAAAGTATTAAAACCCTTGAGGCATTTTTGCATATTTATGAAAAG
AATACTGAATTTTATTCCATGGCTAACTTCCTAAATTTTAGAGCAATTTGCCACCCGGGTCAAGCTGGAGAAGGATCAGAACCTACTGCTTGTC
ACTTTCTTATTTCATCCTGAGTTCCAATACGTGGAGCTATTTTAAAATTTCATTCTGTTATCAACCCTCCTCCATAAACACATTACTGTCACTC
GAGTCCATGTGAAGAGACCACCAAACAGGCATTGTGTGAGCAACAAGGCTGTTTATTTCACCTGGGTGCAAGCGAGCTGAGTCTGAAAAGAGAG
TCAGCAAAGGGTGGTGGGATTATCATTAGTTCTTACAGGTTTTGGGATAGGCGGTGGAGTTAGGAGCAATGTTTTGTGGGCAGGGGGCGGATCT
CAACAAAGTACATTCTTAAAGGTGGGGAGAATTACAAAGAACCTTCTTAAGGGTGGGGGAGATTACAAAATACATTCGTTAGGGTGAGGCAGAA
ACAAATCACAATGGTGGAATGTCATCAGTTAAGGCTATTTTCACTTCTTTTGTGTATCTTCAGTTGCTTCAGGCCATCTGGATGTATACGTGCA
CGTCACAGGGGATATGATGGCCTGGCCTGGGCTCAGAGGCCTGACAATTAGTTAAGCTTACTCTCTGTGTCTTGACCCAAATCATTGATGCTGA
GGAAGAAAATGCACGACCAGGAAGGTTGGCTCTCCACCTTTGCATCTACCTAGCCGTTTAACACTTGCCAAGTTCCATTGTTCAAGCAGCTTGA
CACCCACCAGAGGATACTGTCATCCTTCCTAGACTTCCATAGTTTATCTACAAGGATGCCACTGGAAGGCATTGTCTTCAGTGAAAGCAAGATG
CAGCATCATGTCATCACCAATAAAGGGCTTTGTTAGTACCTACTCTGTGCAGTATTTGTCAGGTTGTGGAGACACAAAATAGTGGAAGACATGA
GTCCTCCAAACAAGGAGTTTACAGTCAGACAGGAAAGTCAGGAATTGTACCTTTGTCATCACCTGGAATTAAAAAAGTAGCATAGCATATATTC
CAAGTTTGAACAAGTTTTCTTTAAAAAAAAAAAAAAGTTAGCATTTGCCTGCTCATTTTTTACTTTTAGTCTTCTAGTATTATTTTGGTCTAGT
TTTATCTAAACTGCCTGCTTGCCCTTTCCATTCAGTCATTATATAATGTTTGACATGTGGATTCTGTCTCACTCATTATCCCAAAACCATCGCC
TCCAAAAGTCCCTGGCAATCTCATGGTAGTAAAATGCATTATTTTTTTCCACCTGAATGTTGGTAAACACTCTCTCCTTGACTAAACTGTAGCA
TGGCTTCTAGCAGTTAAGGCCATGTCCCTAGGATGGCCCCCTTTAAAATGCCTGCCTGAGAAAGTTCATCACTGCCAGGAGAATGTCCTGTTTA
TTCTACCCAAAGGCAGGCAACAGGGAGCTGGCCCGTGAACCCTCTCTTAAAGCAGTTACTTTAGAAAGTTGCAATGATAAATCCTTTCTCTGTC
CCTTTGAGATGTAAATCTTCTAACCATCCAGAACTGTCTTCCAAGGATCTGAAAAAAGCAACCCTTCAAAGAGGTAACTCCGACTTTCAGATTC
TGTGGGAGGGAGGGCCTAACTTCAGACGACCTTCCTCTAATTTACACCACAAAAGTTTGCTTCTCCTCCAGGAAAGCTCCAATTAACAAACCCA
GATGGCCTAGTCACACCCACCAACCCCCCTACTACCCCTCAGCGGCACACCCTTCCCCTAGCAGCACATCCCAGTTTTTTAAAAGTCTCATGCC
TTTTGTTCCCATAAACACTGGGGCCTCTTTCCTATTGCCATCATTATGACTGAATATAACCATCCTTCCCACTTTAACTAGTGTCCAGCTTTGT
TAATCTTTGGCAATGTTCTATTCTACACTTCAAATTTATGGTGTCTGAAATCAAAATATTCAAAGTCATTGTTTGCAAAATAAACAGAACATCA
GTCGAACCACGTCAGTTTCCTGCTCTAGAAAATAAAAGTTGAACTGGCCACTCTCCAGGCCAGCCCCAGAATCCAAACACAGATTCCCTGGTTT
TCTTTACAGTTTTTCTTGCTCTTTCCCTACCACCTTGAGTGCCTTCTCCCTTTACCTCTCTGCTTCCCAGACCTTAGACTCCTGAAAGCCCCCA
GAGAGAAGCTTTCCTCCCTAGAATGCTGGTGGGAGTCACTGCCTAGGGTGGGCTCCCAGCTTTTCCTTTTGATGAGCAAAGGCTCTTAATCTCA
TCCAATTACCCCTCTCTGACTCCACAAGCCACGTGTAGACCTCAGCTACAGACTTACACAATTTTCTTTCACATAGGAGGCCTCAAGAAATATT
TGCTCAGAGAATGATTTATCTGCTGAATATAAAACCAGCCCAAGCAACTTGTGTTTACCAAGAGTAATTTTTGTAATGAATTTTTTTTCTGCAA
TGCATGAGTATTAATTTTGTAATCAGCAACATAATAAATATTTTATTTTTATAAATTCAAGAGCATGAGCATGAGATATAAGCTTTTTTCTTGC
TCTGATCCAAATTACTTTGTATTTCAAAATTTGCGGCTTTAAATTAACAAAAAATTTCTTAATCCCATTTAAGGTGGGCCAAAAAAAATCGAGG
CAAGATCGTCTCAGTCATAAGTACAGCAGGGCTGGAGACAGAAACCTGTCCTTAGTGGTGCTACGTGCTAGTAAGACTTTCCAATAAAACCAAA
CAAAACTGCATGATCTTTCTGTTCCAAGGATACTGAAACAGATCAGTTGCTAAGTGAAATTGGTAGAAGGGTTTTTAAACTGTAAACAGGTCAG
TTTAACTGTAAATGACTATTTAAGTCACTGTTTACAAAATAAGTCCTATATCAGATGGGAAGAATCAGGAATCACCTCTCCCTGGGAACAAGAC
AGCAGCTGACCATCCATGAGGATCCAGAAAGTCAGACAGAGGGTGGGAAGGAGTCTATACCCAGGCAAATAAAAATAGCTGCCACTTTCCTCCT
GGAGGGTTTTTCTAATTATACTTCATGAGAGGCAGGATCAACTAGGAAAAGAAAGACAGGAAGAACAGGGAAATTATTGCCTGAGGGAAAAAAT
GCAGAACTGAAGTCTGTGAGTTAATTCCATAACCCAGGGCTTTTCAGACATCTGAGTGCCACAGTCACGATTCTTGTAATATCTTTTACCACAT
CTTTTATTATAATATATACGTAATCTGTTTATTTTGTTTGTTTATTTGTTTGGGGGGAGACAGGGCCTTGCTCTATCACCCAGGCTGGAGTGCA
GTGGCACGATCTCAGCTCACTACAATCTCCACCTCCTGGGTTCAAGCGATTCTCGTGCCTCACCCTCCCGAGTGGCTGGGATTACAGGCACGTG
CCACCACACCCAGCTAATTTTTGTATTTTTAGTAGAGACGGGGTTTCACCATGTTAGCCAGGCTGATCTTGAACTCTTGACCTCAAGTGATTCG
CCCACCTCGGCCTCCCACAGTGCTCGGATTATAGGCATAACCCACCACACCTCGCCAGTGTGTTTCTTTTAATTGTCCCTAAACTGACTCATTT
AAAATAAAATTAGCCTTGTTCTAAGGAATAATATCCATAAAACCTATGATCTGAGATGCATAATTTAAAATACAGATATAAATAAACACATAAG
CCTTTTAAATGTTCAATTGTGTACCTCCTAAAATTATGTCACGTACCTGCAGAGAAACGAGTACCATGTTGGAAAACACAGCTGTCATTGAGGC
CAAAGGCTCGAGTCGGAGTCAAATCTAGGCTGGGGCAGTTCAATAGAAAAGATCTTTCTCTACAGTGGTGTTTCTCAAAGTGGAGTTCTTCGGC
TGTGGGGTGGTTTTAAAACATAGGTCCTCCTGTGGAGAGGGAGGGGCAATTTCCCCGTCCTTTGCATCTGAGAGTGCTGGTGACTGATTCAACC
AGTAAGGTACAGTGGAAATGAGACTGTGTGACTTTCCCTGGGACACAATCTCTGCGACCCCAGAGCCTCATGTAAGACATTGGCGCCTCCTCTT
AGACCACATAAAGTTGCTTGGGTCCCCAGTCCCTAGTTGCACTGACCCCACCTTTCCAGCCATCCTCACCAAGCTGGCAGAATGTAGAACCACG
GGGAACATCACCACCTCAGCAGAAGAATACAGGGCAGAGAGATCACCCAGCCAAGCCTGCCTGAAGTGCTGACCCACGAAATTGTGAGATATAA
TAAAATAGTTGCCTTTTTAACTCATTACATTTTGGGATCACTGGTTATGCAACAGTAGATAACCAAAACACACTGACTATATCACAATCACCAA
TGGAGCTTGTCAAAATGCATATTCCTGGGCCTCAGGTGCAATTCTCCAGGAATTCTGAAGTCTCATAATGGGACCCAGTAATCTGAATTTTAAC
CTAGCTTCCTGGATGTCTCTGGTAGACACTAGAGTTTGAGAGACATTCCTTTAGAGCTATTCATTCATTCATTCATCTATCCAGTCTACCGTCA
TCTTTTTACCATTACTATAAATCTGGCCATGTGCTGGGCTCCAGGCATAACCCAGAGACCATGCCACTGTCCCTGCTCTCAGGGACACCAAGGA
CCAAGCTGGGCACCTGACAGTCAAGCACTAGTTGAAGGAGGAGACCCTGAATCTTCTTCCCTGTAGCAGACTGAGGTACCAATGCCAGTTCTCT
CAGAATTGCTATAATTTGTTCAAGACCTCCCAAACTTACTCCAAACCACTAACAATCCGTCATGGCCAAGAAATTAGGAGAAGATTGAATTCAC
TGATATTTTGGAAATAGCCATCTCAGCAGAACCTTGGGATCTCTGAGGTTGAGCTTCCCTCCCGGACGGTTTCTGTCACCTTTTATTATAAGAA
GCAAGCCAAGGTCTCTGTGGGCAAGGCAGAAACAACTTCAGCCTCAATCACAGATTCATTTGCCCTTGAAAATGACATTCATGTATATTTTCCC
CCAAGTAGAAAAGTGACCATAGATAGTTCATTACAGAAAATGAGAAAATAAAGAAAAACATTTCAAACACACAAACAAAGCTCCCCTCAGGCAT
CTAACCACAGTCTAGCAGAACGTTAAGAGGTTGCTTGAGTCAGGCACACCTGGGTTTCAACCTCGCCTTCATGTAAGCTCCCCCAGGCTTGGTT
TTCTCCTCTGTGATTACATTGCCTACTGTGCTAGGCTGTTGTGACGATTAACTAAGAATATGTGCACATGTGTGTATGTCATTATCATTATCTT
TGCTAATAGTCACAAGTATTTCTTTCTAGTCTGATTCCCTTTATATGCCAGTATTTATTGTTGGTTACTTAATAGTGCCTTGAGCCCCATCCCA
TCTGTACTCCCCAGCATTCTAATGTCCCCATCAGAGGCAGGGCTGTGTCCAGAGGAATCCTAGGTGGAGAGATGGTGGCGATGGAGCAGCAGGG
TAGGAGTGGATGGTGACAGGCCAGAATTTCCACGATCACTCCAGCGCTCCCCCTTGACACGTCCATCTCCCTCAGAGGTTTAGGGAAAACCCAG
AGCCCAGAGAGCCTGACAGCCTCCCTGTGGCTGCTCCGTCTTGATGTGTGGCTATGGCAACTGTTCTTTCAAGTTTTCTGGGGCAGTTCTACAT
GTCCTGTAGTCTAGTTTAGTCCATATAATATTGTACATAAAATAGCAAAACACAAATTCTATGTGGCAGCAGCAAGGCAATACGTTTACTGCTG
TTATATAGGAGGTCCTATTCCATCTGTATTTCTGCCCTTGGGAGGAAATCCATTTTCTTATTATTATGATACCCATCCAGATGTTACTCCCTCT
GCAACAAGAGGAAGTGAGCAGCTCCCTCCTTTTCCAAAGAACACAAAGTCATGGATGGCCAAGCACATAGACTTTGATGAGAACAGCCATAGGA
TCAAATCCCTGCTCCACCTACTAATTATTCGCTGGGTGAAATTTTTAACATTTCCAAGGCTATCTCTTCCCTTTTTTTTTTTTTTTTTTTTTTT
TTTTGAGATGGAGTTTCGCTCTGTCGCCCAGGCTGGAGTGTAGTGGCACAATCTCAACTCACTGCAACCTCCGCCTCCCAGGTTCAAGTGATTC
TTCTGCCTCAGCCTCCCGAGTAGCTGGGACTACAGGCGCGTTCCACCACGCCCAGCTAATTTTTTGTATTTTTAGTAGAGACAGGGTTTCACCG
TGTTAGCCAGGATGGTCTTGATCTCCTGACCTCATGATCTGCCCCCCTCAGCCTCCCAAAGTGCTGGGATTACAGGCATGAGCCACCGCGCCCA
GCCTCTCTTCCCATTTTTAGATGGGGATGATCATATCTATCTCATGCCATTATTGATAATTAAATGAAGTGATGTATAAAGTGTGCCTACCAGA
CTGTCTAGCATATAGTAATATTCTAATATTGTTAACATTTCCAGTATTCGAGTCTTCAGAGCAGAGAGAAAACCACTGTACCAGCAAATAGACA
AACAGCTGCCACAAGATAGTGATAACAAAGTATCAGCAGAGCACACTAGAGGCCATCGGTCTCCTTAGTAATACTGCTCGGTAAGGGCTCGGAG
CTGAAAGTCAACAACACACAGAGACTGTCAATTGCCTGTGAGGGGCTGATGGCAACCCCCACACGAGGAAGCACATCACAGCCCAGATCTAGCT
CGCCACATGCAGAGGATCAGCCGAACTCAGGCAACACAAGACGATGGGAAATACAGTCTGGGCCACAACACAGATTCTCTCAACAAAATTTCTT
AAATTATTTGTGGCAAAGGACCAGTTTTCAAAAAATGTGTTGAAGACCAATGCTTCTTTTTTTTTTTTTTAAGCCACAATAAAACTATTTTGGT
GATGTCAAATTACAATTAAAGTTTCTAAATGCTGACTTACAATTTCTGAACTTACCTTGTTGGGAACTAGTGGCAACCAATACTGAGCTCTGAG
TAGCATTGGCTTAAGAGGTTTTGTTTGACTTTTTGGTTTTTGTGTGTGCTTAAGATTATTATTGCTCTTTTGTTTTGTTTGCTAGTTGCTTATT
GAACAGGAAGTGGGAGTTTAAATCAGAGCCTTCCTTCATATGACGTACAGCCTGTCATCCACAAATTTGTTTGTATTAATTAAGAAATGCATAT
GGTTCTCACCCAACAATGTGAGCGCAGACATTCTCACACTCTCTCAGAGAATCCCAGGAGAAGAGCCAGTAGCGGGAGTGGTAAGAGTCTAGGT
GGTTAGGGGTCCCAGGGTTCCTCTTTAACATACCCTTCTCCCTCTCAGGGTTAGGTAAAACTCTATTTATTGTAATAATATTATAATAAATAAT
AATGTATCATAAGTATAATAATATAATAATAATTTTTCCCTCCAAAAGGCAGAAATATAGATGGAAAATATAGATGGAATAACACTTCCTATTA
ATCCTTGATCCAAAGAATCCTTCAGGAATTTTATTATAAATAAATTTTGAAATAATATGGATCTACAATAGGGAAGTGGTTATCCATCTTCCTT
TAATTCTTGTAGAATGTTGCTTTGCCATGACTTATTTCTGTTTAGGCACATCCTGCCATCCAGATGGTCTCAGCTCTGACCCCAGCCCACACCT
CTCTCTCAGCACCAACCTCAGATCCCAACTGTCAACCAGATATTTTAATTTGGATCTCCTGCTGTCCTTCAAATGCGGCATGCCTGAAATAAAC
TTCAGTTCCACCGCTCCACCCCTTCCCCCAAAGAGACGCAACTTCCTAAATCTGATTTCTACCAAAAGTGCCATTACTTTACTCTGATTAATAT
GCTGGAGTAATATTTGACTCCCTCCCTGAATTCATCTTTCATAGGTGAGCTGATGCAGATGTCTACCATATTTCTCCTCTGCTTTTCTGTGCCA
GACTGGGTTAAAATCATCTCACACTTACACAATCATTTCATCTTCCTAAGTAGTTCCGCAACCCTCCCTACCCCATTACGCTCCCCTCCAAATC
ACTGTATGTACCAGACTCGTCTTTTAAAATAACACTGTAGTGCTATCAATGTTCTGCTCACAAAAGCCTTCACTGGCTCCACAACCCCAGAAAA
TAAGATCAAACCTCCTCAACAGGCTTCTAAGACCTTCTAGAATGTAGTTGTCCCTACGAATTCAGCCACATCTACAATTACTTCCCAATATAAA
CTCTCCACTCCACCCAGGCCATTCTCCTCCCTGTCCTTCTGGCCCCTGACCCTTTTCTTCCAGGACGAACCCTAAGTTCTGCCTTTTAGGTGTG
TCATTCAGGACTAGACACAGTCCCAGCTCTGCCACCAACCAGCTGCCTCTCCCAGCTTACACCATGTTTTTTTCCTTCTCTGAACATGGCAGAT
CTTCATCCCTCATGTCTTGTCATAATTCACATCATAAGCCATCTGTTTCATGTAACATACTAGTGTCTAGTAACTTCCTTTTTCTTCTTTGCTA
CCCAACCTCCTCCTACTACCATCACCGCCACCACTATCACAAGGAATCTAGGGTTGTGCATATAATAGCAAATTTAAAAATACCTCATGAATCA
GATTCTATCAAAAAAAGAAGATGGAGAAAGAGGAAATCAAGGAAACAAAATAGGAGGAGAAAGAGAAAATATTGCTATGTTTTCTCTTCGATGC
TTGGGTATGCTGTGTGCTCATGCTATTGCTCAGATTTATAGACTATTTGAACTGAAAGGATCTTAAAAATCACTGGGTCCAATAATATAATGCT
GAATGTGAAGACTTATTAAAAACTAGAAATAACATATCTGGCCCAGAAAAAACAATAAAAGAAAGATGAGTATCATTTACCAGCTGTGTGACTT
TTAGAATGTCACTTGACCTCTCTGTTCCTCAGTTTCCTCATTTTTGAAGTGTGAATGATGTAACACTTATTGAGGAGCTAAATGAGATGATGTA
GGTAAAGCACATGGCCAATGCCTTAAACATGGTAAGTGCTTAGTACCTGTTAAGAAAAAGATCCCTGTCCATGCTTCGAAAACATGTAAATCAA
AATTTCTTGAAGGCTGGTTATAAATAATTAATTTATTGTTAATAATAAACTGTGAGATCATTTAATGGGGTGTTTCTATGGTCTGAATATTTGT
GTCACCCCAAAATTCATAAATTGAAACCTAATCCCCAAAGCGATGATATTAGAAGGTGGGGCCTTTGGGAGGTGAGTAGGTTATGAGGGCTCCA
CCTAGTGAATGAGAAATTAATGTCCTTAAAAAAGAGGCCCGAGAGAGCTGCCTTCCCCTTCTGCTATGTGAGGACACACAGTGGGTGCCATCTA
TGAAGAATGGGCCTTCACTGGACACCATATCTGCCGACCCCTTGATCATGGGCTTTCCAGCCTCCAGAACTGTGAGCAATAAGTTTCTGTTGTT
TATAAATTACTCAGTCTAAGGTATTTTGTTATAGCAGCCTAAATGGATTAAGACAGTTGCTTAATCCATGTAAGCATAGCTACATCTCTACCTA
TTTCTATGTCTACTTTGGATCATAATATAAAACGTATTTCTAACTGTGGGTCACTGTCAAACAAGTTTGAAGGCCATTGGATTATTAAAGTCCT
CCACTAGTAAAAATGGAGCAGCATGCCAAAACCTCATGTGGAATGCTAGGTGCTACTTTAAGAGCAGTTTTACCTTCCATCTTTTCTGGACCTT
GGTTTTCCATCAACGCCTCAGTCCTGCTAATGTTGTCTTTATTGTTCGTGCATCTGACATGTAACCCGGACCACTGTTGCCATACTAATTAATG
TAGAGAGAATTCCCTTGCATGGAGCCCTAAATTCTCCACACCTATGGTTAGGTCTAGCATGTGTCTTTCTCCATCAGATGGAATGCTCAGAAAC
CTTGAGGAAAGAGGAGAGCTTCTGATTTTGAAAGATGTTTCAGCTGGTAACTCTGTCTTGCATTTTGCAGTCATTCTGTCACCCACATCGTAGC
AGAGAACAACTCGGGTTCGGATGTTCTGGAGTGGCTTCAAGCACAGAAAGTACAAGTCAGCTCACAACCAGAGCTCCTCGATGTCTCCTGGCTG
ATCGAATGCATAAGAGCAGGGAAACCGGTGGAAATGACAGGAAAACACCAGCTTGTTGTAAGTGTCATGGGTGTGATTTTCACTGTTCTTTGCT
TGATGGTTAAGAACATAGGCTTAGGATCAACGGAGCTGGGGTAAACCCTAAATTCTCTGCTTACTACCTGTGTGACCTTCGGCAAGTCACTTAG
CTTTACTGAGCTTCAGTCTCTTCCTTTATAATATGACATAACGATAATGCCAGCCTCACAAAATGGTCATGAAGATTAGAACAACACACATGTA
AAGCGCTGAACACAATGTCTTGACTATAGCAGGCATCTAACGAGTGGTCCCTATTTTCCCGAACACATTTATACACAAATCTTTCAAAATATTT
AAGCCTTGATATTCTAGCAGCCAGAAAAGCAGGAGATTCCTTTTTTTCTCTCACTGTGATCATTCAGAAAACAAAACAAAACAAAACAAACAAA
ACAAAAAGTTAAGCTCTCAATTCCAAATATTTGAGCAATTGTGCTAAAATCTTTGGGAAGAGGGCTTGGACCATAACTACAGAAGTTCATAGAA
AACGAAGATGATATTTAATTGATAGGTCTTTTCATTTTTTTTTCTGATTTCAAAAATAATACGTGTTGATAATAGCAAGTTCAAATATTAGAGA
TGTATATAACATAAATTTTGAGTCTCCTATAATTTTATCCTCCAACTACAGACAACTACTTCCAAATTTTTTCCGTACATATCTGTTATTACTA
TTAATTTTTATTGAAAATGGATGCTTATGCATTTGTTTAGGTGAGAAGAGACTATTCAGATAGCACCAACCCAGGCCCCCCGAAGACTCCACCA
ATTGCTGTACAAAAGATCTCCCAGTATGCGTGTCAGAGAAGAACCACTTTAAACAACTGTAACCAGATATTCACGGTAACGGGACTTTACATCA
ACACCCAGGGCAAACGAAGGGCTTGCAGCTTCTTTCTGTGGACCGCAAATTAAATTATAAATTTTTCTGAAAACCTGGGAAAGTTTTCCTCCCA
CCTACCTGCACCCCTACCCTGATCTCTGATCCTTCTATCCTTCTTCTTTCTATCGAGACTGGGTCTCCTTCCTTCCTGGAAATAGACAAATACA
TTTAGGTTATATTCTTTAGAGACAGCGGATTACTATTTACAGATAGTTGTCATTAATGGGCGCAACACTTGTCAAAAGCAAATATTCTGAAAAA
ACAAATACTGGAATTAGTCCAAAACATAGTAAGAGCAAATTACATAGAAATGGTAACAATACATCAACAGCTTTTGCTGGGAAACCAAAAAATT
GAGCAAACCTAGATTCCAGCCTTTCTGATATTCACTAGTTTGGGCAAGTCAATTTCTCTCTCTGAGCCTGTCTCCTCATCTATTCAAAAAGAAA
TTGGATGGATCATATCTAAAGCTCCACTCAGCCCTCAGATTGGTAATTGTGTTTTGAATGGCCCTAAAACCCACAACTGCTTGTCTGCTAGCCT
AGAGTCAGTGGTTAGCTGGATGAAACATGTAGTAGTAGATCAATCCAGAGCTTCTCATTTCTAGACTTCCAGCTCCATGTCTGTGTTGGAAAGA
CTAGGAAATTGTAGCTACTGGATATGCTGAGCATGAGATCACACAGCTACAGCTGCTTCCTCCCCTGCTAGAAATACAAACGTGCCCTACCTTA
AATAATCCTGGCTCTGGGTCTGTGTGACCTACGACTACACTCATTCATTCAACAAGCCTGTGTTAAGTGCTGTGTTTCATGGGGCTGCTCAGTG
ATCAAGGCAGAATTCTAGCCACAGGCTAGTGGGAAGATAGTACAGAAGCAGGGATTATGCTGAGTCATGACTAGCAAGATGCTGTGAGCACCAA
AGCATTTTTTAGTTAAATCATCTGTGAATGTTTAATAGCAGTGTACTCCAAGTTTCCGCATAATGCACTCTGGTACCTGTCTTTTCAATTCAAC
TCTACTCCATGACACCAGAGAGGCATACAAGGGTATGGGAGACCCCATCCTGACCTAGAAGGAAACACGCAAGTGGTATTCCAATATTTAAAGG
AGAGCAAGAGCCGTTTGTGAGTGACCACTGGCTCAGGGGCTTGGAAGCAAATCTCCTGCTTATCTGGTTTTATCCTGCAGCATGCCTTTGATAT
ACTGGCTGAAAACTGTGAGTTTAGAGAAAATGAAGACTCCTGTGTGACATTTATGAGAGCAGCTTCTGTATTGAAATCTCTGCCATTCACAATC
ATCAGTATGAAGGACACAGAAGGAATTCCCTGCCTGGGGTCCAAGGTGAAGGGTATCATAGAGGTAAGGGTGAAATGGGATTTGTCCCACTTCC
CAATAGGTAGGTGGGAACGGGGGAGAGAGGGATGTAGCTAACAGATTTTCCTGTCAACTATGTAGATGTGGTGTACATCACAAATTTTCCTTTA
TACATATTCCTCTCTCTCTCCTCTGTCTCTCTCTCTCTCTCTCTCTGTTTATTTTTATACACATACACACACACATACACATATATATATGTTA
GGGGTTGTGAATATCCTACTCACATGGCACCAAAATACGTTAGTGGTCGCAGGTGTCAGAGTTACCGATAGTGAATCCGTTCGGGTCTGCAGCA
ACCCCAATTATTGCTTCCGCAGAAGAAAGAATTCGACTGGGGGGCATAAGGCAGAAAAAGAGACTGAGGCAAGTTTCAGAGCAGGAGTGGAAGT
TTATTAAAAAGCTTTAGAGCAGGAAAGAAAGGAAAGTACACTTGGAAGAGACCCAAGTGGGCAACTTGGAGGACAAGTGCAGCGTTTGACCTTT
TGACTTGCGGTATATGTTGGCATACTTCTGGGGTCCTGCGTCACTTTTCCCATGATTCTTCCCTTAGGGTGAGCCACCCGCATGCACAGTGCCC
TCCTTGTCCTTGCGAGGCGAGCATGTGCAGTGTGTCTACTGGAGTTGTATGCATGCTCACCTGAGGCATTCTTCCCTTTTCCGGTGGAATGCTC
CGGAAAGTCATACTCTGCCATTTTGTCTCTTAATGTGCATGCTCATGCCCACTCGCCCAATTCCTGAGATTTTAATGGAAGCTGCTGATTACCA
ATTTCAAGTGTTTTTATCTATTGGGAAATTCCCTCTCCCTGGCGCCAGTTGTGACTAATCATTTTATAGGGTGACAACTGCCAGACCATCACGG
AATTGCCTCTCTCTAGCACTGGCTGCAACCAATTATCATTTTAGAGAGACAGTGTGATAACTCCTGGACCATCACCTGATGGTCGCCTGACATT
CCTGGTTTGGAGGGGGGAGCCCTCTCCTGCCCTGCTCATGCCTGACTAGCTACCTACTCTAACATATAAATAGATACAGACGTGTTATTTATAA
TCCATACCCTAATATTATCACAGCTGGAGCTACATGGAACCAGCTAGTAGAAGCAGAACTCTCCCATACTGTTATTAATCACAGTGAAAGACTC
AAGTGGCCTAAATCTTGACAGCAGGGAAATGGTCTGATTTCCCTCAGTTCCACCTGGGGGCGCCACCTTAGAGCAGTGGTGCGTTTGACATGAA
GTGGAGGTGGTTCTACACCTCTCCAGGGAGAGCGCCAGAGGTTGTGCTTATTTACTGAACGGTGTTTAGGGATTCCCCAGCAAAGCAAGTTGGT
CATCTGGTAGTAAGTTTCTGTATAGTCAAGTGCAAGGTATATTATTCAAGGACAATTATCCCAACTCTACTCAGAGGAAGGGGGCTCTGAGCTA
GACATCACAAGGGCTTGAATGTAGATTGGCTTTTTGGTGGGTCCAAAATTAACCTTGTCAATGGAAAAGTCTTCTTATTTCAGTAAGGTCATAT
TCATTGAAGAGAAAATTATCACCAATATTTGGACATACATTTTTCAGTGAATATTTTGGTTTGGGATTCTGTAGATCTGGATCCTAACCATTTG
TATCTTTGTTCCTAAGGATCGTAAAGGGGTCCTTGGATATATGATGAGCTAGCATTTTCAAGATTGTAGGGAAATGTCTTGTTTTCTCCAGATC
AATTAGGGAAATACCCCTGCATGTGATGCATGCACATTTCATGAGAAACCAAACTACTAGGTCCATGAATTTGAGAGTCTTAGACAGTAATTGT
CCAACATCACTAATTTAAAATATTTTTCAACTGTCCATTAGGAGATTATTGAAGATGGAGAAAGTTCTGAAGTTAAAGCTGTGTTAAATGATGA
ACGATATCAATCCTTCAAAGTAAGTGATTTTACATATATTTATTGAAAATGGTTTTTCAGTTAATCTTATTACTTATTCTGGCTGGTTGTATTA
GTCAGCTTGGGCTGCAATAACAAAATACAGAGATGGCGTGGCTTAAACAACAGAAATTTATTTTCTCACCATTCTGGATCAAGATGTCAGCAGG
GTCAGTTTCTGCTGAGGGTTCTCTTTCTGGCTTCTTCCTGTGTCCTCACATGCTCCCCTGCTCACATGGTGAGAGAGAGACAGAGAGAACAAGC
AATCTTTCTGGTGTCACAAAGACACTAATCCTGCCCCGGGCACCATAGCTCATGCCTGTAATCCAAGCACTTTGGGAGGCCAAGGCGGGTGCAC
TTGAAGCCAGGAATTCGACCAGCTTGGCCAAGATGGTGAAACCCCATCATTTTACAAAAATACAAAAATTAGGTGTGGTGAGGCATGCCTGTAA
TTCCAGCTACTTGGGACATTGAGACATGAGAATCACTTGAGTCTCGGAGGCAGAGGTTGCAGTGAGCTGAGATCACGCCACTGTACTCCAGCCT
GGGTGATAGAGTGAGACTGTCTCAAAAACAAGACAAACAAAAAGACACTAATCCCATCATGAGAGCCCCACTCTCATGACCTCAAAGGCCCTGT
CTTAAATACGGTCATACTGGGGATTAGGACTTCAACATACAAATGGGTTGGGAGGGGGAATTCCGTCCATAGCTTTGGTCTAGGAAGAAAATGC
CTAAATAAATTCAGGTGCCCAGATTATTGCCATGCTGTCGTGATGTACAAATTGCCTTACCCTCCAAGAGTCTGTCACTCATGGGTACAAACAT
CATGGGACATGAGGACCATGGGGGGGTTGGTGGGCACAGGACGGAACTGCTCTTAAAGAGCAGATTTGTGTCTGAAAATCTCCTTACTGCATCT
TCATTTACTTGGAAGCAGGGTTTATTACCTCCCAATTTTCCCTACCTTAGAGTTGCAAGTGGAAGGGACTTCCACCAGTTCCTTCAAAGCAAAA
GTGATGTACAGGCCTAAAATATGGCCATGAGCAGGAGCACAGGCTTGCAGTGTAAACAGACCAGGGTTTAAGTTTGGGCTGTGCCACTTCCCAC
CTGTATGATGGTGGAGAAACTATATAACCTACATGAGCATCTCTTTTCAAAATGTGACGGTACCTATGCAGAGCATGAGTGTCGGTGACAGACC
CAGCAAAAGGGAGAGCCCTATAGAGCTAGCGTATCACTCATGGACAGCACAGGCAGGCCGTCAATTACACTATACCTTAGCCCTGCACAGGTGC
CAGAACCTGCTTGTTACTTCTGTTTCATTTTGTAATGCCACATGTCAAGAAAGGACTCACCTGAAATAAAAAATGCCCTTAAAAATAACCATCA
CCCACCTGCTCGTTATATTCAGGAGAAACTAGGTCAAAGATTTGCTTATACTTTTTCTTCATTCCCTGAGACCTAGAAAGGGTCATGACTCGGA
TGTTATAATGATTATCTTAGAGACTGATGGCATGCCTTTCCCTCCATTTTAGCTCTTTACTTCTGTATTTGGAGTGGGGCTGAAGACTTCTGAG
AAGTGGTTCAGGATGGGTTTCAGAACTCTGAGTAAAGTAAGGTCGGACAAAAGCCTGAAATTTACACGAATGCAGAAAGCAGGTAAATTGTCTC
TCCTAAAAGTCATTCTTGCTATGGGCTCGTCGGGTCCTCCTGGAGCTCTAAGTCACTTACACTGCAACTCCAATCTGGGAGAGCTGGGACACTT
CTTTGATGTCCATTGATGCTTGGATCTAAATCCTAGCTCCAGCAGTTTCCAGCTGTGTGATCTTGAGCAAGTTGGTTAAAACCTTTGAACCTTA
GTTACCTAATCTATGAAAGAGAGATAACATTGATACTTAGCTGAGAAGTCAATCAGTTAATCCAGGCAATGCCTCAGCACTGTGTCTGACACAG
AGCAAAGGCGCCCTCCACTGTGGCTGTTTGTGGTCCTTCACCCAAATGTCAAGCCAGGTTGTGCTGCTGATCTGGTCCTTACTGACTCTTCCGG
ACATTGGGTCCTGGCTGTCTCCCTGGGTAAGAGTTGTGGGTGAAGAGTCAAAGACAGAAGGGTTGGCTGCTAAGAATACTTGAACAGAGTTGTT
GCCTGGCACAAATGTGAAGATGCCCTAGAAACTGTAAAACACCAGGAAAATAGAAGGGAACTGGTGGAAATCTAGGCACAGGGTTGGTATACCC
AACCAGGACTCTTTAGCCTAAGACAAGACCGTGGTGGGACATGAGTGCTGTCATCACCCACCTGAAAGCCTGGCAGGGCCTCTCAGAGGTTGAA
GGTAGCCTGGTCACTTCCATTTATGAGGCTCCCAGTAAGTTTTTTAAAAATGAAGGAATGGCAATACAGTGTGAAAAAAACTGTGTGTTTAAAA
AAATGTTTACTTTTAACAAGCAATAAAAATACAATACAGTTGTGATTCAAAAAGGAGTCACAGCCATAGTCCCTACTCATTTTTTGTAAAGTTG
CTAATCTTTGAAAGATTGCTAATAATACAATGCCACATAGGAAATAAAAGAGTCTAAGAAAAAAAGATCCAGTATTTATATTCCAGGACCCTGA
AAGCACACGTTTAATAAGGATGATTTACACCAGTTCTCAAGATGCCATTCAAAAGAAATTTAAATAAGTATTAAACATAAGTAGCAAGTAAATG
GTTTGGAATGATGAGTAAAGACTCTCAATGCTTAGAGAGAAATTGGGAGGCAATATAGTGTTTCATTTGGATGAAACCCTTATCAAATGACGTC
CAGTGCTCTCAGGATCTAACGGCTTCCAATTCCCTAGCTTTGTAAACTGGATTTTTGTTCTTCCTGTTGAGCTTTTCCAACCCTTTGAATGAAA
CAGCTTTATTCATTTAGTCAATCAGTCATTTCCATCTAACTGTTTAATGCTACCAGTTCTATGCAGAAATCAAGGAATGCCCTCCCATTATACT
TATACCCCCACAAGTAGTTATAGGCACCCCTTGGAGCAGCACTACAATGAATGGAATTGCCAATGGGAATAGCTTAGAATACTGTGAAGGGTAA
GCTAATTGAACATATTCAAAGGACACTTCATGGTCAGATGCATAACCTGATCCAGTTTTACTCATATTTGCACTTGATTGCAGTTCCCTGAGGC
TTGAGCGTGCACATGCCACGTTTGCCTTGAAAACTCAGTTTATGAACTATAGCAGCACATATTTAACCTTATACTGTGTGATAAGTTTCCTGTA
ATAAGTTAGTCGCCTTGCCATACCAAAAAGGAAAGCCAATTAAATTACTTTCATGGATGTCAACAAATCATCATAGTCTTACATGTTCATTCCT
GATAGCTGCACATCTTCTTAAAGAAATAGTTGTGAATCCTGACTTTTGGATCGTGTATATCACTGCATAAGCACAGGTGTTTGTGGTTACCCAG
TGAGTTGTACTTTGAATATTAGTCATGATGACAGTTTCTGTATTACAGCCTAATAGAGCATTGCTTCTTAGATCAAACTCCTCATGCTCACAAA
AATGGCCATGTTGCAATAATTTTTAGAAATGTGGCTTTTTAATAAAAATAAAAAATAAGTATCATATTGAAATAAAGAAAGTAGTGTTCCACAA
GTCGCGTGCTTTGGTATTGGACACAGTTTATAGGTGTATTCATATGAATACCATTTTCTCTTCGTCTTGCCAGCATCTTTCCGTGACTGTCCCC
ATTAGAGATAACATGGGAAATTTGGCAGGGTGTGCAAGCCAAATGGCCACCCTGCTGCCTCCCTTCCACAATCCCAGGGTGAGAAACAAGGACC
AATAGATTTACTCTTGTCATTTCAGAGGGCCAGTGGCAGGTGAAATCGCAAAGAAGTAGACTTAGGCTTTTCAAAAGAAAGAACTGGCACAACA
GAGTTTACAATTAGGAAACAGGCTCCTCTGGAGCTATCAGAGTTCCCCATTATTAGAGTCAAGCAAAGTTTGAATGGCCCCCTCTTGGAGACCC
TAGATAGGAGAGCAGCGATGCTGAAGCATGATTTGATGTCCCTCAGGCTCTATGGATCTAATCACCAGGCTCATCTCCTCACCAATACTTCACC
CCAATCCTTGGAGATAAGCCTCCCTTCTCCTTTCCTCTTTCTTTCCAAATCTCCCTCCACACTGCTCTGTGGGAACCACCAAGTTAGCTTTTCA
TCATAACAACTCTGAACTTTCATTAATTCATGCTGGTCAGGCAAAGAAGCAACAAGCTAAACCACCTAAGATATTTCCTGCCTTGCTTCAGACA
CACATCTACTCTGCTCCTATTCTTGACTTTAATTTTTTAATGGATCCATCACCTTCCAACTCTAAATCTCCTCATCCCAAATCTTTATGCGTTA
CCTTTTAAAAAACAGTAGGACACAGCCTATTGATCAAGAAGGCTGGTTCTTCAGTAGGAAGTCAGATCTGAACAGTTTGGATTCCAGAGGGATG
TGAAATATTGTCTCCTTCTCAGGAAATTGTAAGCCTCTAGGTTAGAAAGCAAAGAACCACTGTTACCCTGAAAAATTCACAACCAGGAGAACCA
CATCGACTATTCTGTCTCTGTGGGAATGGAGTTTGCTGTTGATGCCTCTAGTCATGTTAGCTCTATCGTGGGGACAGAATCCCCTTCTACTCAG
GGCTGGTTGTTTCACACACGTGTCACTCTCTCCATTGACCTGTCAAATGGCCTCTCAGGATTTCTGTATTATGAAGACCTTGTCAGCTGTGTGA
CCAGGGCAGAAGCAGAGGCCGTCAGTGTGCTGGTTAAAGAGGCTGTCTGGGCATTTCTTCCGGATGCTTTCGTCACCATGACAGGAGGGTTCCG
GAGGTAAATAACTTGGGTCGCTTTGCCTCCTCTGCCCGAATACTTCAGTGGCTCCCTGACTTGGACAGGCATCTGAAACGGCACAAAAGGCTGT
GATCTGGTGCCAGCTTCTTTCTACAGCTTTATCTCCTGCCTCTCACATTTCATTTCAGCAACCCCAAATGGCTTGTCATCCCCTGCACATTCAG
AGCTGTTTCTTCCCTGTTCCTTTGCTTACTCAGACTTTTCTGCTTTGACTGCCATTCCTCAAATTGTCGCTTGGCTGACTTCCCCTCATTCTTT
AACACTCAATTTAGATGGCACCTTCTCCAGAAAGCCTTCCTTGACTCCCTCTTTCTCCATGCTGGATTAGTGCCCCTCTTCCATGCTCCCTGAG
TACCTGCAAAGCCCTTGATCCACATAGTTACAGTGATTTGTTCCTGTGTGTGGTTTCTCCACTAAGTTTGTAGAGCTTAGTTCTCAGCAAGACT
AGTTCATGATGCCTAGCACAAAGTAGGTGCTCAAGAAATCTTGGTTGAGTCAATAAATGACTGACTGAATGAATTCATGAATGAGTGAACGCTT
CACAAGGATGTATGTTGTTGTTGGATATTGAGGCCTGCATTTCAGAAAGGGATTTCTTTCCTCCCACAGCCTCCTCTACCTCTTTCTCCAGCCC
TCTCACCTGGTGGCTTTTGGCCATCCTAACAAAATAGGTTTGGGGCAAGGAAGAAAGTAAGAGAAGATCAAATCTAAGATAATGCCTTTACTCC
ATGTCATAATTCCAAGAGCGCTACCTTGTATTATGTGAATACAATTCTTAGTTTGCTTGTTGTCATCCTTGTCTCCATCTGTGTATCTGTGGGT
GCATGTAGGTTTGTGTGAGTGTGTAGAATAAATAATTTTTTTGTTATTTTTTGCCTTGATATACATAATCAGATACATGCTATGTATGACTTTT
TCTGTGTCATAAATTTATTTTGTAAGGAACAGCGATGTGTAACCAAGGATATTTTGTTTCTTAAAAAAAAGTCAAGAACCTTCTATGTTGCAAA
TCTTAAGCATAGGGCATAGAAACAAAGGTGATTTTTTAAAAATGAAGACTAATATCTAATTGATTTCCATTTTATACTGCTTTTGAAAATTAGG
GGTAAGAAGATGGGGCATGATGTAGATTTTTTAATTACCAGCCCAGGATCAACAGAGGATGAAGAGCAACTTTTACAGAAAGTGATGAACTTAT
GGGAAAAGAAGGTGAGAAGAAAGATGAAAAATACATGCACACGCAAACATTATGTTAACACTTGAATTTTGCACATTACTTGCCTTATAATTGT
GTAATGACCATCTAATCAATTCTCAATTATTTGTGGCAGCTTATACAAAGCCATATGACCTACAATAAGACTTTCCTGTTTTGGGTCCTACAGT
ACATGTTATGAATATGTCCTGTATCTTGTTTATTATACTTACCCTATTGCCAACAGGGCCAAGGGTGGCTATGCATTTCTGTTTCACATGCAGA
GGCATTAAAGCACCCAATCAAAGTGAAAAGTATGCACTATGAAGACCTGGGAAATGTAAGCTGGAAAGCTGCAAGCTTGGGTGAGGGGGCAGGA
CTGCAAAGACTGGGATAAAATCAGTCTAAGAGGTGTTGATAGTGCCTCGATAGATTCGAGGCACTTCGTGCTGAGATGGAATGAAGGAAACTAA
TGAGGGCAAGCTGAGCCCTGCAGAGGTCATGGCTCAGAAGCCCCCATACATTGCAGAGATCAGACTTGTGACAGGCCTATTGCCTGGTGGAGGA
AACCAAAGTAAGCTAAGCTAACCCTGCAGCCAGAGCCACATGCAAGCCCTGACTCCTGTCCAACTCTAGTGAGAGCCTCACTTCCTAGAGAGAC
CAGAGGGTCCTACCCTTGGGACCTGTAGATAGGTCTGGCTGGGCTGGTGTTTCTCCTCACAGCTTCCTCCTTCCTGAGTAGTTTGCATCTGTTT
GGTGTGGACCAAGCTTACTGATATCCCAGAAAAGGAAAACCTCAGATAAAGTGAAAATAAAAGCTAGAAACACAACCCTAGGGCCAGGGGCACT
GCAAGTGAAGACTCAACTTTAGAACCAGATCCAAACTGACACGAGTTTGAATACTGGGCGTCGAGTCTTCCCAGAAGGAGTTGGAGGGAATTAG
AAGCGAAGGGCAGCGAGGGAATCCTGGCTAGACAGCCTCACACTCAGCCTTGCAACCCTCCTCAGAAGGCACCTCAGACTAAATTTGGACGGAG
CCTTCCTGTCCGAGGTCAAGCTCAGTGCATCTCCCTCCTGCCCCACAGCTCTCTTCCTGAGAACTGGCCCCTCTGTACCCTGGCGAGGATGGCA
AGGATGGAGAGGTGGGGAAATGCCTCCTCCTAGTCAGGAGTTTCTCAGTGTCCAGTAGGAAAAATAAAAGAACATGCTGATGTCAATATTCAGT
CAACCAGTGGGTATTGATGCGCAAGCCACAGCCCAGAGGGCTGCCCTGTACATAGCCGGGGCTGGAGATCTAGAGGTGGCATCAGGGGTTGTGT
GCATGTTTGACAACTGTGGCCTGCTGCAGACAACCTTCTGAATATGCAGACACGAGCATTACGTTCAAGGATGCATTCTCAAACACCTTGCAAA
ATTCAAGACAAAACCCAATAAAGAATGGCCCATTTCTCTCTTACCCACCAAGTGACTGTTTACTGTTAGGAATCCAGCTTTGTGGGCTTTTTTT
TTTTTTTTAAGGGAAGGTGTGTTATTACAAATTTGGCTTTCATGTGAGACTTGTTCATATGTTTTATATCTCCTTTTTCAAATTTACATAATTG
AAATTTACACAAAAGGCTGCTATTCTGTACTAGTTTCAGCAAGTTTCCTTAAAAAGTGGAATTGTATTTTCCGGAGCTCGTGATTCGAGGTTAA
CTACGGCAACATCTACCACCTCCTCCAGAGGTTCTTCCTAACATTAGTGCTTCCCCCTTCAAATGGTGTTTTTTCTTCAAGGCGTTGAAGGGTG
AAGAGTTCCCTCTATTGAGGTCAACAAAATAAAAAGTATCTTTTTGAATTGAGATAATGTCTTGTGCAGGTTTGTAGAAGAAAGACAAAGCATG
ATTCCTAACAATAATCAATTTACCAATGATGCTCAAAGTGTGGTCCCTGGTCCGGGAACACCAGCATTGCCTGGGAACCTGTCAGAAATGCCCT
CCCTGCCCCTCCTGCATCACTCTGGGGACAGAGCCCTGCAAACTGAGCTTTAGCAAACCTTCCAATTGCTTCTGATGATAGCGCTTTTAAACAT
CTAACCTAAAGAGTACATGGTAACAAATCACACATCCCAGTAGCTTGATGACATTATTTGAGGGTTACTTGCTGTTTTTGTTGCCAGATTTAGT
GGTTGCTAGCACGTTTCTCTCTCCTGTCCCCACATCTGCCCTGTGCCATCAGGAACTCCCACCAAGAATGGCAACTCGATTGGGCACATCTTAG
AATCACTTGTCCACTTTCTGGATAGGCAACTTTAAGTGAAACATGTGTCATTAACTTTATCCCACTTTCTCCAAGGCAGGTGGACTAACACTCT
CATGAGAAAGAGTCTCCCAAGTGCTACCTAGAAATAGCAAGTCATCTTGCCCCAAAGTTAGACTCAGCCTCCACCCACAACGCTTTTGGCTGGT
TAAACCTGGTCATCAGTATAATATGAAGGGACACTGTCTTTGTCCATTTACCTTGCTATAAAGGAACACCTGAGGCTCGGTAATTTATAAAGAA
AAGACGTTTACTTGGCCCATGCTTCTGCAGGATGTATAAGAAGCACGACACCAAGCATCTGCTTCTGGTGCGGCCTCAGGCCGCTTACACTCAT
GGCAGAAGGCAAGGAAGAGCTGGCAGGTGCCGACGTCACAGGGTGAGAGAGAAAGCAAGACAGAGTGGGGAGGGGCCAGGCTCTTTGTAACCCC
CAGCTTTCACGGGAGCTAACAAAGTGAGAACTCACTCATTACCATGAGGATGAGGACGGCACAAGTCATTCATGAGAGATCTTCCCCTATGACC
CAAAGACCTCCCATTAGGCCCCACCTCCAACATTGGCGATCAAATTTCTACGTGAGATTTAGGGGGACAAACATCCAAACTATAGCAGACACTA
ACTCAAAGCTGCTCCCCAGAAACAATATGATTTTTTAAGTTTTATTATTTATTTATTTCTGAGACAGGGTCTTGCTCTGTCTCCCAGGCTAGTG
TGCACTGGTGTGATTACAGCTTACTGAAGCCTCAACCTCCCAGGCTCCAGGGATCCTTCCACCTCAGCCTCCTCAGTAGCTGGGACTATAGACA
CGTGCCATCATACCTGGCTAATTTTAAAATTTTATGTAGAAATGAGGTCTTATTATGTCACCCAGGCTGGTCTCAAATTCCTGCAGCGTGGCTT
TTTGTTTATGGTTGGTTTGTTTTTAAAGGACTGAGGAGAGCCCAGAGCACTAGCTGAGGTTCAGAAGTGGAGAGGGAGATTTTCTTGCAGTTTT
GCCATTAAGTGGCTTTGCTTCAGCCTCTCTCATCGCTAAATTCCTTTTCCGTAAAGGGAAGATAATGCAACCACTTTCTTAGTGTTTTCTCCTA
GTGTTTGCAGGTGGGCCTTTAATGCAAGGCCAAACACATGTCCATGTTCTGCTCGAATCTGAAAAGATTTCATTAAGAATCCAGTGTTAAATCA
TAGATCTTCTTCATCAGGGCCTTTTCCTGATGCCATTCTGTTTCTTTTCAGGGATTACTTTTATATTATGACCTTGTGGAGTCAACATTTGAAA
AGCTCAGGTTGCCTAGCAGGAAGGTTGATGCTTTGGATCATTTTCAAAAGTGCTTTCTGATTTTCAAATTGCCTCGTCAAAGAGTGGACAGTGA
CCAGTCCAGCTGGCAGGAAGCAAAGACCTGGAAGGCCATCCGTGTGGATTTAGTTCTGTGCCCCTACGAGCGTCGTGCCTTTGCCCTGTTGGGA
TGGACTGCCTCCCCCGTAAGTGCTACATGGACCCATCGGATGATGTTAGCTTTCTGAAAGACGTAGGCCGAGTCTACCTGGGCCCAGCGGAGAG
ATGACGGCAACAGGGGCTGGGTGACAGGGGAGGGGCCAGTACCCAGAAACCTCTAACTCAAGGCATCCTGTCCACACATGGCCAAGTATGACAG
ATTAATTTAGAACAGTTATTCCCAGTGTGTGGTCCCCAACACCAGCAGCAGCAACCTTACCCAGGAACAGGTTAGACATGGAAATTCTTCACCC
CACTCCAAACCTATGGAATCAAATCAGCATCTCTGGGGATTTCAGCCCAGAAATTTCTTTGGAAAACCCTTCTAGGTCTTTCTGATCCACCTCA
AGTAGGAGAACCACTGAAGTACAGAAACCAGCAAGGGGATTGAAATAGGAAACAATTAATAAAGTGGAGAGGCAACTCACAGATTGGGAAAAAA
TATCTGCACATCATACATCACATAAGGGCCATTATCCAGCATATACAAGGAAACCAAAAGACTCAATAACAAGAAAACAAATAATCCTATTTAA
AAATGGGCAAAGGACTTGAATAGATATTTCTCAAAAGAAGACATATAAATGGCAAGCAGATATTGCCATCTTGCTTAACATCTCTAATCATCAG
AGAAATGCAAATTAAAACCACAATAAGATGTTATCTCACATCCGTCAGATTGGCTATTATCAAAAACATGAAAGACAGCAGGTGTTAGTGAGGC
TGTGGAGAAAAGGGAACCCTCTTGTATTGTTGGTGGTGTTGTAAATTACACATTTTGGAAAATAGTATGGAAGTTCCTCAAAAAACTAAAAACA
GGGCTACCATATGATCCAGCAATCTTACTTCTGCGTATATAGCCAAAGGAAATGAACTCAGTATGTCAAAGACAGGTCTGCACTCCCGTGTTCA
TTGCAGCATGATACACCATAGCCAAGATATTATATAAAAACAACCTAAGCATCCACAGCAGATGAATGGATTTTTAAAATGTAGTATATGCACA
GTGGAGTATGACACAGCCTTTCGAAAACAGGACATTCTGTCATTTGAGACAACATGGTTGAACCTAGAGGACATTATGTTAAGTGGAATAAGTC
AGGCACAGAGAGACAAATACTGTATGATCTCGTTTCTGTATATGTAGAATCTAAAACAGTCAAACTCGTAGAAGTACAGAATAGAATGTAGAAT
GGTGGTTAGCAGAGGCTGGGGGATGAGGACAGAAAATGGACAGGGAAAAGGGTCAACAAGTACAAAGTTCCAGTCAGACAGGTGGAATAAACTC
TGTTCTATTGCACAGTATGGTGACTATAGTTAATAAAAGTGTATATTTCAAAGTAGCTAAAAGAGAGGATTTTTAATGTTCTTACCACAAAGAA
ATGGTAGATGTTTGAGGTGATGGACATGCTAATTAGTCTGATTTTATCATTCTGCAATGTATATAGATGTATCAAAATACCATATTATAACCCA
CAAATGCATACAATTATTATATGTTAATTAAAAATAAAATCAAGCTATAAAATTTTTCTGAAAGAGCACAAGTTGAGTTCATCCCAGACCTGGT
TGCAAGCTCCTGCTCTGCTTCTTGCTGTTGGGTGATCTTGGACAAGTTCCCTATATCCTGTAAAACTCAATTTTCCCGTCAGGAAAATGAGGAC
GATGACCTTAGCTACTTCCCTGAGTTGCAGAGACATTAGGTGACATGATGTTCTCAAAGCCTCAGCACCAAGCCAGCACGTGGCCAGTACTGGA
TAAGTAGTAGCTTCCATATCCTGTCACTCACAGGAGAGGACACCTGTGATTTGAGTCCCATTCGCATGTGTTCCACCCTCCCAACTAAACACCT
CACCCTGTTTCTCCTGTTTTCCCAGGTAGCTGGTTGTTGCTTTGAGTGAAATGGGTTCGTAGAGGCTTTTTTATACAGATGAATGACACAACAC
AAATTAATTGTTCTCAGCCGTGGGTTAAATGTCAGGTGTCTTTGCTTTGGACTGGAATTAATTCAGCACAGCATCTTAGCACTGGGATTTCTTA
CTCTAATCACAAATGTTGACTGACAACTAAATGCTTTTTTCATTCCTAAAAAAAGGGGAAGAAAAGAAAAAATATACCATAAATGCAACTTGTC
TGATAGACATAAATCTTTGGGAACACATAGTTGATTGAGAAAGGGGAACGTTCCAGAAAGAAGTCCTTGGTTATGCAATGTCATGCCAGTTAAG
CATCACTGGCTGAAGTGCTTGACATTTGCCCATTTTATTTTTAAGATGGACATGTTTTCAACACATTCTAGTCTGTTTCCTCAGAGTACTCACA
AATTGTTGCTGTTGGTTCTGCAAGGCACTGGCAGAGCACTAAGCTCTTTTGCTTTTTAGCTGGGTGACTTTAAACAAATAAAGGACTTCTGATT
GCAAAGTCCATCTCCTCAACCATCCCGGGTGAGCTTTCCTAAAATAGCAAATTGTTCCTTACCACTAGAGGGCACCAGTATCCCAGGCTTGCTC
CCTACTCCCACATGAAGAGGACAAAATTTCTAGATTCTGTTTCTCTCAGGAAAACTAGTCTCCATGACAATTCGTTGCCCTCCTTTCCCAAGGT
GTTCCTGTCCTCTGTCTTAGGCTTCATGTATTGCTCCCTTCAGTCTCAGAGCCCATTCCTGACTGTGGGAGATGTCATCCAGGAGCAGTTGACT
TAGACGATCAGTTTCATGGCATCTCAGTGTGAAAGGATGTTAAAGCCAGAGGGAGTCTTAATCATGTCCTTCCTCCATCCTTTCATTCACTCAA
CCAAGACTCACGGAGTCTGGATGACTTGACAGCAGATGCACAGCCAGGTAGGGACAAAGTCCAGGCTGGAACGGGTTCCCTGACTCCTCCATCC
TCTTTTACAATAACAATATTCCAAATCATGTAACACCCACCAAACTAGTATTTATCCAGCATTTACTCTCGACCAGGCATTATCCTGGTAGAAC
GCTCACGCTATTCATTTATTCATTCAATAAAACATTTGCTATACATCTTCTACATCCGAGGCACTATTTGAGGGAATACAGTTGTGGACAGAAC
AAACCAGGTTCCTGCTCTCACAGAGCTTACATTCTAGTGTGTTCATCGGAGCACGCACTTCTGTCACCAGAGAATTTGGCCCTGGGGGAGGAAA
ATCACTTTGAGAATGTTTAGTGGAGTTACATATTTCACTAGAGGGATGTAGCCACCTAATACTGAAGACATTCTAGACGTGTTAATAATTTATT
TTAACTATCTCCTATCCAGCAGTTTGAGAGAGACCTCCGGCGCTATGCCACACATGAGCGGAAGATGATTCTGGATAACCATGCTTTATATGAC
AAGACCAAGGTACAGTTCTCTTCCTAAAAGGGGCTACTTTGATCCTCATCCCCAAGGCTGGCCCCCGAGCTCTTTTCATGGACTGATTTTAGTA
TCCAGGTATGGTTTCCTTTGTCATGCGTGTTCTATTTCCAGTTCATCATAGTTGAGGGCAGCAAGAAAGATTTCCACAGACTCGCAGTTTCCAC
CCGGCCCAGAATGCAATTCGAGAGATTTTGGCTGAAACATGATGAGCTAATTGGGCTTCCAGAACAAGTCCAGCAGTGAACATTAACTGGGTTT
GGTATATCCTCGGTGTTGGCAATGAGTAACTCCAAAGTAAACCAGGGATCCCCAGAAATCACAAATACTACTTGTGTACCCCTCAAATGCAGGG
GGCAGTGGCATTTTGAGTGGCCATGTAGGCCATTAACAACAGTCTCCTGCAAGAAAGAGAGCAACAGAGCTGCCCTGAGACATAAATGAGCATC
CAGCTAGTCTCATTGACTACCCATGTCACTCATTTGGTGTGCTAGTCTTACTGTGGTCATGATCCAATTCATCCCTCAGCAAAACGATAGATGC
CTGTAGAAGTTAGAAGAGTTAAATATCCAACTGTTAAATCCACAGTTCTAAGGGCTACTCTTCTTACAGATATCAAGGACACCAAAATAGGCTC
CATGGGGAAGAAAACTCAAAATAACTAGAAGTAATGGTAGTGGCCAGGCGCAGTGGCTCACGTCTGTAATCCCAGCACTTTGGGAGGCCGAGGC
GGGTGGATCACTTGACGTCAGGAGTTCAACATCAGCCAACATGGTGAAACCCCATCTCTACTAAAAATACAAAAAATAACCAGCTGTGGCGGCT
CGCACACACCCAGCTACTTGGGAGGCTGAGGCAGGAGAATCACTTGAACCCGGGAGGCAGAGGCTGCAGTGAGCCGAGATCACACCACTGCACT
CCAGCCTGGGCAACAGAGTGAGACTCTGTGTCAGGAAAAAAAAAAAAAAAAAAAGTAATGATCATTTCCCTCTAGGAGCTTGTAGTCTAAATGA
GATGTATTCAGTTAGAGTTGTCTCAGTTACAAATGTTAGAAATCAAATCTAAAGTGGCATAAACAAAAAAGGGAACTTGGGTTATATACTGCAA
TGTCGAAGCGTTCAGGGGCAGCCGAATATGGGTGCTCAGGCAGGCTTCCCTCAGTATTGCCTTTATTCTCAGGCAGGTTTCCCCATAAGTCCCA
CTCTTCCACCTACTTCAACAGAGAAGAGCCTGTTTCCTAATAGTTCCCCCAAGAATCCTACCTGATGCTCATTGGCCAGGCCTGGGTCATGTGC
CTATCCCTGAACCCATTACTGTGGCTCTGATTGGCCAGACTTGGGTCATGTGCTGCTTCTTGGAATTGCCAGCAGCTGCACCTAACCCTCCTGG
ACTGAGAGTCAAGAGGAGTGGTTTCCCATAGALAAATCCACGTGCTGCTCACAGAAGAGGGGGCTGTGGATGCTGGCAGGAAAGCCTCGGATGC
TAAGCATGTGGGTGAAGGCAGATTCACATAGTACAATGAAGGAACACTTACAAATTAATACATTCTAAATAGTTCAATGTAAGCAAGTACCTTC
TATGTGCCTAGCACCGTGCTATGTACAAGAAGGGATGGAGGAGAACAGCTGCTTGGTCTTAAAGAGATCACAGAGTGGGATCCTGCTGGGGTTA
GAGGCAAGCATGGAAAGCTAAGGAAAACCCAACACATACAAGTATCCTATAATTACCAGCAGTACAAAATTATAACTGCATGTTAGAATTACCC
AGTGCGTTCAGATAGATTGATTATAAGATCTCTCCTGAGCTTCTGATTTCACTGGACTGGGGTATGGCTCAAGCTTCAATGTTTATTTTTTAAA
CTCCATCAGGTTGTTCTAATAAACAGCTGTGGTTCAGAACTACAGCCCGTAGGAAAAATATCCAAATGGCTTTTGCTGAATATGTTAAAGCTGT
TATGATTTTACTTTAATTCATTTAGTACCATGTTTTGGAAAGTCAGTTAAAATGACTTTTTATACTCAGAAGCAGGGAATTATGTTTGGGCTGT
AATTCATTCTGACAAGGCAATTTTATAAGTAACACTTTCACTGTGTCCACACCATGCTGCTTATGAAAAATATCTGAATGCACATATTTCTTGT
TATGTTTTCAGAGGATATTCCTCAAAGCAGAAGTGAAGAAGAAATTTTTGCGCATCTGGGATTGGATTATATTGAACCGTGGGAAAGAAAATGC
CTAGGAAAGTGTTGTCAACATTTTTTTCCTATTCTTTTCAAGTTAAATAAATTATGCTTCATATTAGTAAAAGATGCCATAGGAGACTTTGGGG
TTATTTAGGTCTTATTGAAATGCAGATTGCTACTAGAAATAAATAACTTTGGAAACATGCGAACGTGCCACTGGTAATGGGTAAGGTTCTAATA
GGCCATGTTTATGACTGTTGCATAGAATTCACAATGCATTTTTCAAGAGAAATGATGTTGTCACTGGTGGCTCATTCAGGGAAGCTCATCAAAG
CCCACTTTGTTCGCAGTGTAGCTGAAATACTGTCTATCTCTAATAAAAACAGCACGAAACAAGATGATGGAATCTGTTTTTCTTCACTCAAATA
TTATAAACTATTTTAAAACACAAGTGTAACCAAGTGGAAGAATAAAGAGCTTACAGTTTGTCTTAAGGGGACAAACAGGTTTAATTAGAAGATG
CAGAGATTCCAGAGGGGATTTGGGTCCGTTCTGGAGCTTGTGGAGGAGGGGTGCTGCCTCTGGACTGGCTCACCTCGGCTCAGTGCCTGCTCCT
TCCCGGCCATCATACAGCTGGTTCCCTAAGCATCCCACCACAGGACCACATAAAGGATGTATTCTCCCTGCCATCGTGCTGAAGGAAAGACAAT
GTCCTAAACCATCAAAGAAATATCTCCCTCTTTTTTCCATCTTGGAGCTTCTGTCTACACCTTATGGCTCAGTCTCTCTCAGGAAGTCAGTCTC
TCTTGGCTTCTTATCTGAGGGCACCCAAGGGAAGGGAGGGAGCCCTGGCTGAGGGAAATTTTGGAACTCCCAGGCTCCAGACCCATTCTTTCCT
CCTGCCTCCTCCAGCAGGCATAGACCCTTTGCCTTGAAGGCAGTTAACAGGTCATCTCTTCCAGCTGCTTCCGTCTCTTCAGCCTGCTAAAGAG
CTGGGAGGCCCATGACGACCGTTTTCCCTTCCTTCCAGTCCCTCTGGGAAGAGGATATTGGGAGATACAATACCTTTTGGCTATTGTGACTAAT
GCTGCTGTGAACATTGGCCTACAAGTATCTCTTTGAGTCCCTGCTTTCAATCCTTTTGGTACCCACCTAGTAATAAAATTGCTAGATTCAATGG
CTATTCTATACTATGTTTAACTCTTTGTGGAACCACTGTACTGTTTTCCATAACAACTGCACCGTATTACATTCTTAACCAACAGTGCGCAAGC
GTTCCAATTTCTCCACATCCTCAACACTTGTTATCTTCTGCATTATCTGTTTTATAATAGCTGTCTTAATGGGTGTGAAGTGGCGGGTGAAAAC
TTTTGCCCATCCTGGAACCCAAAGACAGTCCAGGCTGAAACTAACACACTGAAAGGGCCTCAGATGGTGTGTGTGCCTGTCACATCTTGAGTTG
ATGGCTAAAAAAAGCTGGGGACAACAAGAGGGTGGACAGCAGCCACCCCCACTGGCCTCAAATCCCAAAATCCACAAAGTATTCTAGTCTTTTC
TGCTGCCCAGTGTTTCCCAGTTATGACATTCAAGATTATTCTAGCTAGTAAGTAGATGAATATTTTCATACTGATATAATCAATAAATATTTAT
TGGGTGCTTACCATGTCAGTCATTTTTCTAGGCACAGGCCATAGAAAAGATAACAAAACTGTCAAAAATCTCTGCCTGTATGATTATAAGCATA
AAGCTTATGTTCTAGAAAGGTGTTTATTTGTGTTCGAAAGGTGTTAAGAAAATGGTTCCTTATTGATGGTAGTGATATAAAGCTTCCTTTTTGG
AATAGGTTTATTTATATAACAAAAGCGACTCAAGGTCAGGGAAATCATTTAGGAAATATTACCAGTGAAATGGGGGAGTGGCAAGAATGAGGAG
AGAGGGACGCAGGTGGCTCACATTTGGGAAATGTGCAGGTGGCCTTGTCGCCTTCACTGAGCAACTCCCCAGCCCTCTTCTCTGGAGGTCCCCA
CCACACAATAGCCCCAGGAGGTCCATGCCAGAGGAGTTTCCCCATCAGAGAACCTCACAGATTCTAAATGCTACTTTCCTTATTTGAAAAGGCC
TGTTGGAGATTCCACAAATGCCATGTCTTTTTTTTCTTTTTTGCTGTAAGCCAGAATTTTTATCAGTTCTGGACATGTTTTTTTTTTTTTTTTT
TTTTTTTTTTTGCCGTAGGCCAGAATTTTTTTCAATTATTGTGGTGACTAGTTACCAGAACTCAGCCTTCGCCTGTGCCTCCTACTTTGGCAGC
AGACCGCCCTCTGATGGCTGACAGAAGAATCCTTGGAGGCAAGCACAGTATGATATAGCAGATCAAGTAGGAAGCAGATTATCTGTTAAAAACA
TTGGAACTATATGTAAGAAGCCATGGATCACCGATGCTCTATTTGTTCACTTTTTTCCCAGCCTTTTAAATCTGTGTTTTATTTGGGGTAGTTT
TTATTGCTGTATCTTCAAGTTTACTAATCTTTTCTTCTGCAATGTCTAGTTTCATTAAACCTCTCCAGTATTTTATCTCACACATTATAGTTTT
CATCTTTAGATGTATGATTTAGTTTTTAAAAACTATCTTCCATGCTTCTATTTAACATATTCAATTTTTCCTCTAGCTTATTAAATACATGGAA
TATAGTGTAACTGTTTCAATGTCCTTGTCTACTAATGATATCATCTGTGTCAATTTTGAGTCAGTTTCTATTAACTGAGTATTCTCCTCATTAT
GGGTCACATTTTCCTCCCTCTTTGCAAGCTTGGTAATTTTATATTGGATTCTAGACATTGTGAACATTGTGAATTTTTTCCTGGATATTTTTGT
ATTCCTGTTAATATTCTTAAACTTTGTTCCAGGATGTGGTTAATTTACTTGGAAACAGTTTGATGTGTCCTCATCTTGCTTTTGAGCTTTGTTA
GGCAGGACTCATCTTACTTTTGAGCTTTGTTAGGCAGCACTAACGTAGTCTAATCCAGGGTTAATATTCTTCCCCACTACTGAGGCAATACTTT
TCTCAGTGTTCTGCCTGATGCCTGGTGAATTATGGTGTTTTCTACTCTGGCTGTTTGGGAACAGGCACGGTTTGTGGCCTCCCACTGAGAGGCC
TGGCCCATTACAGCTTAGGGTATTGTTCCCTCTAATCCCTTTGTGTGGTTCTTTCTCCAGCCTTGGGTAGTTTTCTCACATGCATATGCTGATC
AATGGAATAATCAAGAGGTCTCCTCTGTCTCTGTGCAACTCTTTCTCCAGTACTCTGCCCTGCAAATTCTATTCACCTTGACCTCCATCCACTT
CCAGCTCTATCTCACTAACAGCATGTGCAAAAGCCATGTGGTGCAAGGACCATGGTGTCAGAGAAAACGCCCACGTGGCAGGAGGGCCATGGCA
TAAAATGAGCAGAGAGAAAGCTCTTGCAGCTGGAGGTTCCCACGTGTCTGAGTTGCAGAGAGAAAGCCTATGTTTGTGGACAGACCATGGCATC
TGAAGGGCAGAGAGGAGGCCTAAGTGGTTGGGGAATAGAAACTAGAAGAGGTGGGACACAAGACTAGGCTTTCCCCAGCTCTCAAAACAAAGAC
CCATATCCTTACCTTGGCTTTCATAAGTGCGTAATCCCTGGCCACGTGAGTCAACAGTAATCACTGTTTGACTCTTCATGACAAAATCAGTCAC
ATTTATGGTAGAATTTTGTCAACATGGTTTGTTCTCGCTCACAATCAAATCTTTCAAATCATTTTAGCCTGGAAATGAAAGTCTATGATAAACT
GATTCTCTTTCATTATAAATCAGATTTTAAGCCCCAGGTTACACTCACCCTGTCCTCCAGAGGCCCCCTTCAGTTTTAGAAATAGTTCTACAGC
CTATTCGACAAACTCGGCCAACTTCCAAAATGTTCCCCAGTCTCACCTGTCCAGTTTTAGTATTTTTCACCCCTTAACTTATTGGAGTTCAGGT
AGCCTCTCTATAGGATGGAGGAGAGAGGTTTTTTTCTGAGTTTTACTTGGAGGTACAGCACTTTAATGTATGCCCTGACATAACCAGACATTTC
AGCCATTTGGAATTCTCAGAGGATTGAAGTGCTATGCCCATTTCTCCTTCTCCTCTAACTTCAGGTAAAGGAAAAAGAAGAAGAGTATAGATTC
TTATCTTATTTGAGCCCGAGAGAGATAGTTTTATGTCTTTGAGCTTAGAGGCAAAGCACCTGTATAAAAGCTTTCCTGATATTATTTCCTTGGA
TGGAGATTCACATTCAATTGGGTAGAGAAACGTTTCCACAAACACCCAGTTTAGCATTTTCATTATGCCCCCAACATTGTCTTACGACCTGGCA
GATTGTCTGACATTGGAAGGAAGCCAGTGCACATTTGTTAAATCAATTCACGCATTAAGCAATAATTCTCATCAAGATGGTGGGAGGGCAAAGG
TTATATGACTTCACAATCTTTCAAAAATTAGTTATCACTTTTTGGTCTTCAATTAACATCGCAATGTTGCAGGGAAAAACGCCTTAAGCAAAAC
TAATAATTGTTATTTCTTGGCAAAATAAAGAAGAAGTGAGTATCTGTCTCCACCAGCAAAGTCAGGAACTGAGAAGAATCATAGAGGGTATAGA
AGGCTGTCCTGATGGGAGTGAGGAACAGGCAGGCAGAAGGAGTGCTTTGCAGGGTTTGGGGAAAATCAAAGTAGAATGTTTGGACTGGATGGAT
GTAACTTTATAGATAAACCAATTCAAACACCAGATGATACAGGAGGAAATGAGACCCACACACATCGTGCAACCTGACTAAGGTCACTTAACTT
ACCAGCAGCCCTGGGTTAGGGAAGCAGAGCTTCTGACTTTGGGTCTCATCATTTTGCTTACCACACAGAGCTGCCCTCCATGAAAGACTGAATG
TCTTCTAGCCTGGCAGAGAAAGTGCCAATATCGGGAATATCCCTGGGATAATCTTGGTTCCAGGGACAGAGCCAATGTTCTCTAATATAGGCAA
AGAAAAGCCACAAACATTTTGTAGGTTCATAAGAATGGCCATTTTACTTTTCACTTCATTCCAACATAAGTTCTTGTGTATCATAGCAGAAGTA
AATGACAGAAATGCTGAACCTTTCTAGTTTAATAGAAACAATGTTTAAATATTCTAAGCCGTGGATTATAAGTAGAATTAAAATAAGTGATCCA
AAACACAAGTCATCCCTCACTTTATAGCTACATGGCTTAGTCACATGCATATTTCAATCCAGCCTTACAACCACCCCATGGGCTAAAAGGATGG
AGGATGAGAACTGCTGTCTCCACTTTACAAGTACAGAACTGAGGCTCTGGATGATCAACAATCGGCCCAAGGGCTGACACCTATAAGTATCAAA
GCCAAGGCCACGGCTGAGTTCTCAGTAAATCCAAGGTCCTTCATGACATGTAGGAGGAAGTACAAGCACCAGGATGACTGGGAATGTCTGTTTA
AAAATGGCTAAATCTGCTTGTTTAAGGTAATTAGATCCAGAGGACTTGGCACAAAACTGAGTTTCAAAAGGGGCTTTGAAGAGCAGGTATTGAC
ATGTGGCAGAGATTGGGTTCATAGAACATGTTCTTGGTTATTCAGGGAAGAGGGGCATGTTTCTGAGTAATCAGAAGCCCTGTGTGAGAAAGGA
ATATTTTCTAGGATATCTGTGTGGCTCATGGAGCTGCTGGATGAACTTAAGATCCCCTGGACCCAGGCCTTCCCACTGGGAGGCCTGGCCTGCT
AGAGGGCAATGGCTATACATGGGGGAACCAGGCTGCCTGGAAGCCTCCTCATGGGAGTGAAAGTGAAAGCCTGTCCCAAGCTGGCTGAATGGCA
GGCACGTTGCTCCTTGGCCCATTCCTGTGGTCCTGTCTCCATCCCCTTGGGTCCTCTTGATTTTTCTTTCTGCCAAACAGCTTTCCCCTAGATA
TCCAACGACTTGGTTCTTTTCTCCAAGTAGACTTGGTTCTACTCCACAGAGTAGATACAGCCCTCATTTCTAATAATCTTTTCGAAAAACTCTT
GCCTTCCCCGCGGCCCCCCATCTTTACTTTCAACACACACACAGTCCGTCGCAGAACTGCCCTCCTTAAATACTCTCTGATTACAGACACAAGT
AGGACCAATGAATCATGTGTCTCCTAGGCCAGGTGTGTGGTAGGGTAGCTGACACATGTTAGAAGGAAGTCTGCCCAAAGACACAGAGGAAATG
AAACTCATATTGAAGGCAAAAGAAGAGTACAGAAGAAAAAAAAAAAATGGCTGGGGATGGATTGGAAAATGGGAAGAGGAGTTACTGGGATAAG
AGAAAGCTGGAGGGTGGCCTCTCCTGGAGTAATCCACCCAAAATAAGGCAACCTGGTTATAGTTTGCCCAATAGGCTCAATGGGCCAACACCAC
TTGAAGTAGCCTCTCCTTTTCCATTTGGCCTCCTTGGAGACAGTCCTCAGCAAGCTGAAGCGGCAATGCTATGCGTGGGCTTAAGGGCTCTTGT
TTAATCTGAAGCCTTTAAAAAATAAAATAAAAGAAGTTGTAACTTTAACAACTATCCCTAAAGCACAAAAGTTAAAGTTATTTGAGGAACTGCC
ATCAGAGGTATCATAATGTCTTACTTTAAAAATTAAGCAAAATGTTTCATTGTAAAGACAAAGTGTTGTTTTAATATGAAACATGAATTTCCAT
CAACATTAACATCTTTAAGGTTAAATGTAACACCACCAGTCTCTGTATTTGGAATTCGGAACTTGTAAGAAACTGCCTTTTCCTGATTCATATG
AGGACACGATCACACCTAGATTGGAAGATTCACATACTACCAGCAACACCTAGAATCATCTTTAGATAACAATTTGGATCATAGGCCATGGAGA
AAGTTTGTCCTAAATGTTCCCTTGTGTGGCCCTGTGACTATGGGGAACAAATGCTGGTGATTAGCTGTTTTATCAGGCAAGCAATATGGGAAGA
GGTAGGAAGAAGGTGGTAGAGAGAGAGAACTAACATTTTGACTGGCAACTAAGTAGCAGACACTTCCCTTTATTTCTTATTTAATCTTTTCAAT
TATGACATGAGGCAAATATTTCCCCTCCTTTAAAAAAGCAAAAACTGAGGCTTGAAAATGTTAAACAACTTCTCCAAAGTCGTACAGCTGCTCA
GTGGTAGGTGTGACTTAAGAGTCCGTATTCTTTCCACTGCTGTGTGACATGATTGAGCCAGGTGCTTCCCTGTGGTCAGGGAATGTGTGGCTCC
ACATGGTTCCTACCCTGAGATATTCAGAAACAATTAACTGCTTCCTCTCACACATCCTGCATAAGTTTCATTTGTATCCCAACTTGCACCAGGC
TGACCTCAGCAACACTTCCATGAAAGTCAGATCTGATGGCTTTTTTGAGAAAAAGGATATGAAAAAAAAAGATGAGAAGGCAAGACACTCAGAA
ACCAAGCCCCTTTCCCAAAATGACAAAGAAAAGCAAATTCCCACTGGGAGCAGGAATAGGATCTAAGGACCCCATCTGGTGACTCACATGTACT
AACTAAAGCACAAGATCTGAGAGTTGGAATTAACCATGTTGGGGATGTCCCCTAAAGAGACAAATCAGCCCCAAAGTGTTCCAGAGCTGTAAAT
GAAATTTGGATTGATTAAGAAGCAGCTTGGCATCTTTCCTCTCCAAGTACAAAACCCTGGGTTTTCAACCCTGTTCCAGTGCCAGGTCTGCTGC
TCCTTGACTGAGCTGCATCATTCCAGGCTCAGTCTGGGCACAAGCCACCACAATCCCCTCCTTCTTTCCATTTCTATAGTAGGACGGTAACGGT
CATGCACAGGTTCCTCGCTTCCTGCTACAGTCTTCACATATATGTGGGCCTCTTGTGCTCCCATCGTATTCTTCTCATGTGTGGGTGATCTCCT
AGGATTCTTAAGGAAACAAATAGGTTTTTTAAAAACTACAGCAGAGAATTCTTTCATACAAGATGGACTAACAGCAACCACATTGACCCTCCCA
CCTGAAATAACCAAAATCAGACACAACATTTGAAGCAATAGTTTTCAAGACACTAGACAACAGGCAAGGAGGGACAATGGTCCTGGAGAGACAG
AAAACAAATGAGGAGAGACCCAGAATTACCTGATCTTACTGCTTTGGGAGAGCTTTCAGGCCACTCAACTATCTTTTGTTTATTGTTAACAAAG
TTTCTCATTTTTTCTCTTTTAACTGTTAATCTATTTGTGTATTTATATTTAAAGTGAGTTTTGTAGGCATCATATGATTGGACCTTCTTTTTAT
AGCCAATGTGAAATCTCTTCCTGTTAATTCGGGTATCTAGACCATTTACATTTAATGTGATCATTGATATGGTTGGGTTTAAATCTACCATCTT
GCTATTTGTTTTCTGTTTGTTCTACCTGTTTGTTGTGTCTTTTTAACTCTTTTTCTACTTTATTTCATATAGATTTTAGAAGAACCTCATTAAG
TTCCATGAAAAAAATTGATTGGGATTTATATTGAAATTACATCTGTTATATAGATCAACATTGTTTGACATCGTTATGTTATTAACACTTTCTA
TCCAGGAGCATAGTATGCAACCCCCATTATTTAGGTATTATTTAATGGGTTTTTTAACCAAAGTTTTATTATTTTCCATAAAGGTATTACATAT
TTTTGTTAGATTTATTCCATAGCATTCTACATATTTTAATACCAATTACCATCACAAATGGTAATTTTTAAAATCACTTTTTCTAATTGTTGTT
GATGTTGAAAGTATAAATTACTTTTATATATTGATTTTTCTATCCAGTAAACTTGTTAAAATTTCTTGTTAATTCAAGTAATTTTTCTGTAGAT
TTAGCTTTTCTTTTCTTTCTTTCTTTCTTCCTTTCTTTCTTTTTTTTTTCTTTTTTTAAGATGAAGTCTCACTCTTGTCCCCCAGGCTGGAGTG
TGATGGTGTGATCTCAGCTCACTGCAACCTCTGCCTCCCAGGTTCAAGTGATTCTCCTGCTTCAGCCCCCCACGAAGTAGCTGGGATTACAGGC
ACCTGCCACCACACCTGGCTAACTTTTGTGTTTTTAGTAGAGATGGGGTTCACCATGTTGGCCAGGCTGGTCTAGAACTCCTGACCTCAGGTGA
TCCACCTGCCTTGGCCTCCCAAAGTGCTGAGATTACAGGCGTGAGCCACCGCACCCAGCCAGATTTAGCTTTTCTATGTCGACAATCATACCAT
CTGCAAATAATGACAATTTTGTTTCTTCCTTTTCAGTGTTACACTTTCTAATTTTTTTTTTTGAGATGGAGTCTTGCTCTTGTCACCCGGCTGG
AGTGCAGTGGTGCGATCTCAGCTCACTGCAACCTCCACCTACCAGGTTCAAGCGATTCTCTTCCCTCAGCCTCCCGAGTAGCTGGGATTACAGA
CGACCGCCACCACACCCAGCTAATTTTTTGTATTTTTAGTACACACAGGGTTTCACCATGTTGGCCACGCTGGTCTCAAACTCCTGACCTCAGA
TGATTCACCCGCCTCAGCCTCCCAAAATGCTGGGATTACAGGCGTGACCCACTGCACCGGACCTATACTTTCTAATTTTTAAAAATCCTACTTC
ATTGACTAGGATCTAAAGTATAATATTGAATAGAAGTGGTGTTAGTGAAGATCCTTGTTTTATTTCTGATCTAAAGAGAATGCTTTCAACATTT
TACTGTTAGGTATGATGTTAGCTGTAATTTTTTGAAAAAGACATCTTTCAAGTCAAGGAAGTTCCCATTCTATTTTTAATTTGCTGAGAGTTCA
TATCACTGTTAAGTTTTTTTGAACTTTCTTCCCCTATTGAGATGGTCACATGAGTTTTCCATTTTAGTTTGTCAGTAAAATAAATTATATTAAA
TTGATTGTCTAATATTAAAGGGAGCTTGAATTTCTGTTCAGCCAATAAAAATGCACTGAGAACCTCCTTTGTGCTTGAGCTTCCTCTTGGTCAT
CTCTTTTCTACATGTGCTCATACTGTGCTTCTTTTGGTGA
HUMAN SEQUENCE - mRNA
ATGGATCCACCACGAGCGTCCCACTTGAGCCCTCGGAAGAAGAGACCCCGGCAGACGGGTGCCTTGATGGCCTCCTCTCCTCAAGACATCAAAT
TTCAAGATTTGGTCGTCTTCATTTTGGAGAAGAAAATGGGAACCACCCGCAGAGCGTTCCTCATGGAGCTGGCCCGCAGGAAAGGGTTCAGGGT
TGAAAATGAGCTCAGTGATTCTGTCACCCACATTGTAGCAGAGAACAACTCGGGTTCGGATGTTCTGGAGTGGCTTCAAGCACAGAAAGTACAA
GTCAGCTCACAACCAGAGCTCCTCGATGTCTCCTGGCTGATCGAATGCATAGGAGCAGGGAAACCGGTGGAAATGACAGGAAAACACCAGCTTG
TTGTCAGAAGACACTATTCAGATAGCACCAACCCAGGCCCCCCGAAGACTCCACCAATTGCTGTACAAAAGATCTCCCAGTATGCGTGTCAGAG
AAGAACCACTTTAAACAACTGTAACCAGATATTCACGGATGCCTTTGATATACTGGCTGAAAACTGTGAGTTTAGAGAAAATGAAGACTCCTGT
GTGACATTTATGAGAGCAGCTTCTGTATTGAAATCTCTGCCATTCACAATCATCAGTATGAAGGACACAGAAGGAATTCCCTGCCTGGGGTCCA
AGGTGAAGGGTATCATAGAGGAGATTATTGAAGATGGAGAAAGTTCTGAAGTTAAAGCTGTGTTAAATGATGAACGATATCAATCCTTCAAACT
CTTTACTTCTGTATTTGGAGTGGGGCTGAAGACTTCTGAGAAGTGGTTCAGGATGGGTTTCAGAACTCTGAGTAAAGTAAGGTCGGACAAAAGC
CTGAAATTTACACGAATGCAGAAAGCAGGATTTCTGTATTATGAAGACCTTGTCAGCTGTGTGACCAGGGCAGAAGCAGACGCCGTCAGTGTGC
TGGTTAAAGAGGCTGTCTGGGCATTTCTTCCGGATCCTTTCGTCACCATGACAGGAGGGTTCCCGAGGGGTAAGAAGATGGGGCATGATGTAGA
TTTTTTAATTACCAGCCCAGGATCAACAGAGGATGAAGAGCAACTTTTACAGAAAGTGATGAACTTATGGGAAAAGAAGGGATTACTTTTATAT
TATGACCTTGTGGAGTCAACATTTGAAAAGCTCAGGTTGCCTAGCAGGAAGGTTGATGCTTTGGATCATTTTCAAAAGTGCTTTCTGATTTTCA
AATTGCCTCGTCAAAGAGTGGACAGTGACCAGTCCAGCTGGCAGGAAGGAAAGACCTGGAAGGCCATCCGTGTGGATTTAGTTCTGTGCCCCTA
CGAGCGTCGTGCCTTTGCCCTGTTGGGATGGACTGGCTCCCCTCGGCAGTTTGAGAGAGACCTCCGGCGCTATGCCACACATGAGCGGAAGATG
ATTCTGGATAACCATGCTTTATATGACAAGACCAAGAGGATATTCCTCAAAGCAGAAAGTGAAGAAGAAATTTTTGCGCATCTGGGATTGGATT
ATATTGAACCGTGGGAAAGAAATGCCTAG
HUMAN SEQUENCE - CODING
ATGGATCCACCACGAGCGTCCCACTTGAGCCCTCGGAAGAAGAGACCCCGGCAGACGGGTGCCTTGATGGCCTCCTCTCCTCAAGACATCAAAT
TTCAAGATTTGGTCGTCTTCATTTTGGAGAAGAAAATGGGAACCACCCGCAGAGCGTTCCTCATGGAGCTGGCCCGCAGGAAAGGGTTCAGGGT
TGAAAATGAGCTCAGTGATTCTGTCACCCACATTGTAGCAGAGAACAACTCGGGTTCGGATGTTCTGGAGTGGCTTCAAGCACAGAAAGTACAA
GTCAGCTCACAACCAGAGCTCCTCGATGTCTCCTGGCTGATCGAATGCATACGAGCAGGGAAACCGGTGGAAATGACAGGAAAACACCAGCTTG
TTCTCAGAAGAGACTATTCAGATAGCACCAACCCAGGCCCCCCGAACACTCCACCAATTGCTGTACAAAAGATCTCCCAGTATGCGTGTCAGAG
AAGAACCACTTTAAACAACTGTAACCAGATATTCACGGATGCCTTTGATATACTGGCTGAAAACTGTGAGTTTAGAGAAAATGAAGACTCCTGT
GTGACATTTATGAGAGCAGCTTCTGTATTGAAATCTCTGCCATTCACAATCATCAGTATGAAGGACACAGAAGGAATTCCCTGCCTGGGGTCCA
AGGTGAAGGGTATCATAGAGGAGATTATTGAAGATGGAGAAAGTTCTGAAGTTAAAGCTGTGTTAAATGATGAACGATATCAATCCTTCAAACT
CTTTACTTCTGTATTTGGAGTGGGGCTGAAGACTTCTGAGAAGTGGTTCAGGATGGGTTTCAGAACTCTGAGTAAAGTAAGGTCGGACAAAAGC
CTGAAATTTACACGAATGCAGAAAGCAGGATTTCTGTATTATGAAGACCTTGTCAGCTGTGTGACCAGGGCAGAAGCAGAGGCCGTCAGTGTGC
TGGTTAAAGAGGCTGTCTGGGCATTTCTTCCGGATGCTTTCGTCACCATGACAGGAGGGTTCCGGAGGGGTAAGAAGATGGGGCATGATGTAGA
TTTTTTAATTACCAGCCCAGGATCAACAGAGGATGAAGAGCAACTTTTACAGAAAGTGATGAACTTATGGGAAAAGAAGGGATTACTTTTATAT
TATGACCTTGTGGAGTCAACATTTGAAAAGCTCAGGTTGCCTAGCAGGAAGGTTCATGCTTTGGATCATTTTCAAAAGTGCTTTCTGATTTTCA
AATTGCCTCGTCAAAGAGTGGACAGTGACCAGTCCAGCTGGCAGGAAGGAAAGACCTGGAAGGCCATCCGTGTGGATTTAGTTCTGTGCCCCTA
CGAGCGTCGTGCCTTTGCCCTGTTGGGATGGACTGGCTCCCCTCGGCAGTTTGAGAGAGACCTCCGGCGCTATGCCACACATGAGCGGAAGATG
ATTCTGGATAACCATGCTTTATATGACAAGACCAAGAGGATATTCCTCAAAGCAGAAAGTGAAGAAGAAATTTTTGCGCATCTGGGATTGGATT
ATATTGAACCGTGGGAAAGAAATGCCTAG

TABLE 25
MOUSE NOMENCLATURE
ICSGNM Znfn1a1
Celera mCG3994
HUMAN NOMENCLATURE
HGNC ZNFN1A1
Celera hCG1736622
MOUSE SEQUENCE - GENOMIC
TTTAGATAATTTTCTGTTGTATCACATTTTCCACTGGTAAATTTGGAGCAAATAAAATTCCACAAGATGGCTCCCTTTGACCTAATGGGGGGAA
AAATGTTTAATTTATCATCCTTTAAGAGACATGGAAATGAGTAGTTGACCTTTTGTAAGCTTATGTGAGACACTCTAAAAAGTGCCCCAGTGTT
CTTAATGGAAACTAGAGGAAGCCATCGACCCCCTAGTGCGAGCCCCACTTTTAGCCTGGAAAAAAAAAGTGGCCTTTTCTCCCTTGTTTGTGCT
GAGTTTTGGGTAGAGTAGAAAACTAAAGTGATAGAGACCTTTCATAAAAACAGATAAACTGTGTCTCTCGCTTCATCAAATGAAAGCCAGCGGC
TACGTATCTCAGGTCCCGGAAGCCAAGGTTGGCAGTTAATTTGTAAAACATGGTTTGGAAAGTTCAAGTTCTTCTCCAACCGAGAGTGCGCCAG
TGCTTTATCATGACGTTATATGTGAGGTTATATGTGTTTGATAGAATTTAAACGATTTTCACTTGAATTAAGAACATCCAGGACTTTTGTCAAA
GTCATGAACTTTTCTGAAAAGGACTTGGAGCATTGAAAACTCATTGTTGCTGTCTGTTGGGAGACTGCAGAACAGGCATTAAAACCGAGCTTCC
CTGTGTTTGGCTTTCTAAAGAGCAGTTTCTGCCCTATTATAAATAGACACGCCGATTTTTGTAACTGTACAAATGAGGAAGTTGTGAGGTTCCA
TGAGGCTCAGCTGATGACCCTTGTCTCCTGGGTTTGCATGAGGACCTTGTAGGCTGAAGGTGAAGGATTATAAAGCCGTCCAGATAGCCTGGGT
TGGCTGGGTACATCTTACACATTAGCCTCAATCTTGTGCATGGTGTGTTGTATTGGCATGACTATCATGAGGTCTGGGACAGGGGACCCTGCTT
CCTAGCCATACCATAGAATAAAACTTTTTAGAAATGTAGACATTCCTTAGGATCTAACTGGAAGGCCAATGCAGGTCGTCAAGTGGCATTTTTG
TCATTGTTCAGTTTATTGTCTGTAGTTGTTATCATATAACTGATTTTCCTTCTGTTGACACTACAGTTGTCTTGCTTATCCCCGTAGAAGTTGG
GTGAGCAAGGGTAACTTTCCAAAGTACTAAAGCCATTAAAAGACCATGATGTGACTTAGGGAACTCAGTTTTTAAATCTAAGTTATAGTACTGT
AAACATAGTTTTGAAAAAATAAGAGAACATATAACAAATTAATATTTTAATAGTTTTTATTAAAAAGTTATTTCATTTTAAGTGACTATGGTTG
ATTTTTATAGGTGCCGTCAATAACACCAACATCAGTTTCAGTACTTTCTGGAAAATGGCCACAGAGTCCATGTGGCCTACCCTAAGTGACAGCG
AGTGACAATGAGCTGGCAATCAGTAAATCATGCTCCTCTCTGGAGCAAAGACTAGATGCTCCTTAATAAGGCTGGGGTGTGCCAAAGTGGCCTT
TTATATTTAAAGAAGCAAAAGGCCAGTGTATGTCACAACAAACTCGGTGATGGTGCTGGCCTGGAGCGTGAGCAGCATCCCATCTCGTTGAGAC
GATGAGTAACATGAAAAGCATGTATGCTGGGACCTAGGAAGTTCTTCTCACTATCACAGTGAACAGTCAAGTCAGAGCACCTTCTCAGTTCCAC
TGGGTCAGTCTGCGTGTAAGGACTTCCAAACAATAGGATACCTCTATGAAGCTCTGTGTGGCTTAACTTCTTTAGGTCCAAACAAAATAAAGCC
TTTGGCAAACAAACTGGCCCAAACCCTTTTAGCTAAAGTAGAATAGCGTTGTAGAAACACCCGCATCAGAGTGCACTTGCTTAATGTTATAGAT
GTACACGTGGTTTCCCTCCACCACATACAGCTTTAAGGTTGTATTTTCAAGTATCCACTTGACTGAATTTACTTGTATGTCTACATAATTTCAC
TGCCGAAAGTCACAATGAGATATTGTNNNNNNNNNNNNNNNNNNNNNNNNNNNNNNNNNNNNNNNNNNNNNNNNTTACTTGGGAGAACCGAATT
CTACCGTCTCTTCTAACAATTACAAACTATCAGCACTTCGGGAATTATCTGTGTGGATCAGTCTTGTGGAAACCGTTGCTGTATTTTAAAGATA
GAAATCTCATTTGTGCTGCATCTCCCTTCCATCACACTCCTCTGGACTACACGTAATGGTCTTCTACAGCAAGAGGGCAGAAGACAATGCTTCT
CAACTTCGGTTTTCCGCCTTAAATGATGAAGTGTGAAAAGCCACTTATGTTTAAGGGAGCTACTTAAGAAGTGGCCAGTGTTGATGGTCCAAAT
CAGAAAGCATGCTAATTGTAACTGCGTGCCTCTCCTCAGTGGCTGTGAGTGGGTGGCTAGTATGGAGGTTAGAGAGGAAACACAGCCTTGTTGT
CTGATCATGGCTTACCTGGGGGAGGAGAGAGCCAAGGCACCACTGACTGTCATGTCAGCACACATGTGAGCCTCTTGCCTCCAATTCCATTGTC
TTTCTTATATAAAATCTACCATCTTTCTTGTTTTTTGATTCTAGCCTAGGGATTCACTCTGATGCCCAGCCTAGCCTGAAACTCATAAACTCAT
ATGTAGCCTAGGCTGCTAGTCCTGGGATTACAAGGGTGAGAGCTACAGTGCCTGGTTCCACCATTTTCTATTACGTGTCACAGAACAGGTACTC
AGTGTTGCCTTCTAATTGGGAAGCAATCTTTATTTTATTCTCTCTCTCTTTCTTTCTCCCCGCCCCGTGTGTGTGTGTACCTGTGTGTGATGGT
CTGCATGCATATGTATAAAGGCCAGAGGTCAAAATGGAGAATCTCCCTTATAGTTCCAGGCCAGAGTCTGCCCCTGACTGGCTCAATTGGCTGT
CAATCTAGCCTCACTGCTCTTTTGTGCCCACCTCCCCAGCACTGGAACTACAGTTGCCTTACTGGGCTTTCTCTGAGGGTTCCTGGGATTAAAC
TCAGGTCCTCACATTTGCCTGGCAACCGCTTACTGACTGAGGTATTCCTGTAGCCCCTTGGCTCCATTTTTAACAGACTTCCTCATTTCCCTTT
GACCCTCCTTCATATCAGTTTCTGCCCACAGAGGGGAAAATGTGAAGCCAGGGCTGCACATCTGAGTGATTTGCCTTCTGTGGCTTAGCTGACT
GGGGGAGGAGGGGCGTGGGGAACACTAAATTGGATTCCTATGCTCCTGTTTAAATCTTGTTTTTCATTGTGAGTTGTACCAGCAAAGCTCTGGG
AGATAATCATGAAAATGTGTAGATTGGCAGCTAATTTTTGAAAAAATGAAAAGAATCAGAGATGAAATAGGAGCGCTCAGAAGTTTTTATGTTC
TCCCAACCTGTTTTGTCAAATTGTTACACAAACCTAGAAGGTCTCTGACTAGATACAAAGACGTCGCATATTGCTGCTCTTTCTCGTGGAGCAT
CCCTTTTGAAAACTACAGAGTAATTCACAGTGATGTGATAAATTTGCAAAAAAAAAAATGGAATCTTCAGTGTATAGATGAAAAGACATGTCTT
TCACAGAATCATGTGTTTTATTCAAGAGGTCTGTGACCTAGGCATGTTTAATAGGTGATAGTCCCGAGCTCAACCCTGTCTTCCACATGCACAT
TTACTTCCTGCCTTCCGAGAAGGACTTTGAGTATTAAGTGGCTAGTCCCAAGTCTCCCTGGGACAGCTTCACCAGCAAGAAAGAAAACCACCAG
GCACATGTAACTGTATGGCCAGAATTTAGTTGCTGTACAAGTTCTGTTATGAGAAGTTTGTCAAGTTTCAGGCTATTGCAAAGAATGAGTTTAT
ATTCTAGCCAAATATGAGGGGCACCTTCCTCTGTCTGACATTTGGATCTTGTCTCCACTGTGGTGTGTTTATGGAGTTCTTAGAGCAGGTAGGA
AAGGCAGACACTGTCATCTGATGACTGGAAGTTCAGAGCTCAGCTCTGCTATTTGTTGGTGGTGGCCTTCGGCAGGCAACCCTTGCTTCATGAC
TCTGCTCACAGCCTCGGTCTTCTCTTCTGCAAAATGGAAATGATAAAGCAGATTCCTGTGACAAATCAGGATTGGCTGCCAGCTCCATGCTGGA
CTCTGAGGCAGGCTGCCTGAGAAAGTGCTGGGCAAGGCAGGTTGTTCACAGTCTCAGGAAGCTCGTTAGCAGTTTAGCTCTCAGATGCCTCGCA
TGTAATGGGCTGCCTCAGAGGTGCCTGTACTCCTCCTTTTCTTGCTATAGACCAGGTTCTCAAGGACCCTTATAAACACAAAAAGTTAATGTAC
ACTGTTTTTAAATCTCAGGACATTTTTCAAAGCTTATGGAAGTACAATACAGGCCAGGTAACAGACTAATGTCTACTGACCTCCACTCCTGCCT
ACAGCAAGCTTTATATGCCATGTGCTTAGCAGTTCTCCTGATAGCCATATTGTCTGGGTAACTTGAAAAGAAAGATGTTTGTGGTTTCTGCCTG
GCCTCGAGTTTTTTCATTTTGATTTAATAAATCTAGGCTCAGCTTGTGAAAATTCTGGGCCAGGCCCTGGGTTATGACATAAAGCACCACAGGA
AGAGGTGCTTGCTGAAGGGAGATCGGCCAGCACCCCTCCCTTGAGGACTTGTTGAGGTTCTGGGCTCTACTAAGTCCTCCTCTGGGCCTTGCAA
GTTAGACATGGGCCAGCACTAAGGAAGACCTGGCCTGGTGGCTTACCTGGAGACAGCAACTGCCCAGGGCTGTTGGATGCTTCTCCCAGGAGCC
AGCCAGGTTCCAGCCTGGTGGCAATGGCCTCTTCGTGGCAAGAGGAAAGGCTCCTGGCCTCCCAGGAAGACTCCCATGGTATGTAGTTTTGAAT
GTGTGCTTCCTGCCCAGTCCCCAGAGCAGTGAGGTCCTTCCTGTGCAGTCTTGAAACCTGGCTCCCAGCCTCCTCTTTCGCGCCTCTTCTCTAA
ACTTGGTGATGTGTTGAATCCTTCTGTCTGAATCAAGGGAATGAAACCTGTTTGTCCCTGTTTAATCAGAAGAAGAGAAAAACAGTCAAGAAGG
AAGAGGGTCAGGAGGGGAGAGAGGGAGACACAGAAATAAAGAGATGAGAAGAGAAGAGAGTTAGATGAAGAAGACAGGAGATGCAAAGAGATCC
CGAGGGATGGTGGGGTTATAAGGAAGGCCTCACTGCTCACTGTCCTAAAGAGCCAGCCTTGGGCAGCCTCTGTCATGCTGGCCTGCACTTATCT
CTGCATCTATTTCCCCTTGTGCAAGATTCAGAACTGCCTGCTCCAAAGAAACAATAAAAGCATTCATGTTTAGAAGAACTGCCCAGGTGAGAGC
TAGTTTACTGACATTGTGGCATGCTCTCCGGTGGGTTGCCTCTATCTGAGACTTGCTTGCTCTTCTCATGCAAAGTGGACCACCACAGCATGTT
TCTGTGACCGGCTTATTCCATCCAATACCCCTGCCCAGACCTGAAGATGGTAATAATTTGCTTTGGTGGTTAATTCCCTATAGTGAATGAAATC
TCCTTGTTTTATTTAAGGCTGAGACTGTAAGATAACTGAGCTCAAACTATTTGTCTTTTTAAGTTTTTAAAATGTATTCGATTTCTCTGACTCT
CTCTCTCTCTCTCTCTCTCTCTCTCTCTTTCATACAAGCACACATGGGGCAGGCAGTCTATGCAAGTGTATCTATGTATGAGTGTGTGTGCATG
TGGCATGTGGAGGTCAGAGGATAGCCTGTGAGAATCAGTTCTCTTCTTCTACCACGTGGCTCGTAGGGATCAAACTCAGGTTGCCAGACTTGAG
GGCATCACCATTATCTTTTAAGACATCTCTCTGGCCCTTTAAGTCCTACCCTTCCCCCTCAGATTCCCTGTATTGCTTTCACATGGCAGCAGAT
GTACTTTGTAGGTGTTTTGTAAGCAGTGCCTGGTGGTCAGCAGAGTACCCAGGATGGCCTAGACTCCTTCAGGACACTTAGGGATGTCCTTTGT
AAACTTTGCCTTAAAAGACCCCAAATGCATTTATCCCCCACTCTGCATTGGTAGGTTACTTCTTTAGTCTCTACCCCAAAAGATGCAGATCTTT
GGTTTTGGGACATTTGAGGGGCTTAAGAAGTCTCTGCAGAATTCGTAGGACACAGTACAGACTGACTCCAGCCCTTAGCTTAAAAATTATCAAG
TACCTGAAACTGTTGTTCTGAAACAAGGGACCCTGCTGGCAACCATACTAGTCACAAGTCCAGAGAGGGTGGGGTTGAAAGTGTGCATGCAAAT
CTAAGGGAGGTAGACTGTGTCACAAGGGTCCATATGCCCTGGCTGGCAGGGAAGGGAGTCTGTCGCTAAGTGACCCACAGAGCAGCCATGTACA
GAGGTGACCTACTGGGATCCTCACATGCACCTGTGTTACCACAGTACCCACTGGAACTTGCTCCAAGCACTGTTGGTGACCTCACTTGTCTGTC
CCCCTGAGGCATGACAGCTGGCTCCTTTGTCCATGAGATCACTGTTTGGAGAAGGGCAGGCACTCATACTCATGGCCCTGATAAAACATTTGTT
TCTATTTGACTTGGAAGTAGCTTGGGTATATTATCTGGGCTATCACTGCTATCATCTAGTGAACCAAGCCTTCAATGCATAGCCATTAAAATAC
AATAATGGCACTGTATAGTAGTTAAATTAAACATTTTAGCAGTACNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNCCATCCACGTATACCATAAAAT
ATATTAATGATAAAAAAGATATATAAAGTTTTAATTATATAGATAACATCACAAAATTACTTATATAGCTATATGGCATGTGTATGTATACATA
TATAAAATAAATACTTTCATATATTAATATAAAACTACATGTACATATACCATGGCTTACATGTGTTCCGTGTTATATAAAACTATGTATATAG
GTGCCCTTGCCTACACAGTTTCCCAAACAACTGGAATCCCCCATCAATTTCCAGGTTTCTTTTAAACGTAGGCCTCAACCCATAGCCCCATAAC
TGAACCAAAGAGATGGAGTACCTAGAAAAAAAAACAAACAAAGGAACCTCACTTAGATTCACCAGATAACCTGAGTCTGCTGACAGCGATAGAT
GTCTAGCAGACCACGGTGAGCACTTTCAATGAAGCCACCAGAGTATTCTACTGGACATAGATTTCTTATCGAGCTACTGAGTGTTGCATACTAA
TAACATAACATCTAAGGGTACCCTAGTGCCCCGAGGTGTGGCCCAGCTAAGTCCACTGCATTCATTTTTCTCCCTCTCTGTCTCTAGGTCCCTC
CCTGATATTTGTGGTAGTCTGTGTACAGTTTATGGAGCACTCACCTGGCCCACTATAAGGCAGCCTTTTCCTATGGGTGTTTTCAATTCAAACC
AAGATGAGGAGCCAGCCACAGGCCTGCAAAGCAGGAGGTTTGAGCCCTCTCCTGGCTTGTTCTGTGATCTCTGTCTGTGAGAGAACGCTGGTGG
AGTCTGCAGCTAGAGCTGAAGAGTGGGGGACAAGGACAGAGCGTGTATTGCCCATCTGCCAGGCTGGAGGTGTATTCATTATTGATGGAGGTAG
TGCGTTGCTGTTCAGATATGCAGCCTGCCTGGGTAAATGAGACATTCTTCAGCAAATTGCTTCGTTTTTTGATTGCTGATTCTACGCGTGTCAC
CAAGCTGACTCAAGGTTCATCGATGCATGCTCAGTAAATTAGAAAGAACATAACTATGGATCAGCCAAGAGAATGAATTCTGTGCCTACAATGA
CCCAGGGCCATTTAATTTTCTGCTTAATTTTGTTGCAGTCAGTTTGCATTTTGGGTTATTATGCAGTAGGAAATTAACAATAAATAACAAATTT
GGTCCTCCTGTGCTTGTAATGATATTTTTATAAATCTTTGTAATGCTGTTTTTTAAAGGATCAAGGTCTGTGCTAGGCTGATACCCCAGCAAGT
ATGTGAAGAGGAAAATGCATTATTCTTGCTAGATAGCCTTGTTGTTAAATAGCATAAGCGTCTCTCTCTCTCTCTCTCTCTCTCTCTCTCTNNN
NNNNNNNNNNNNNNNNNNNNNNNNNNNNNNNNNNNNNNNNNNNNNNTCCCTCCCCATATTTTTGGTTTAAAGTAAAATCCATTTCTCTCTTCTC
TTCTCAGATAACCTGAAGACAATGGATGTCGATGAGGGTCAAGACATGTCCCAAGTTTCAGGTGAGACCTTGGGTGATAGTTGTATGTCCAGAA
GTTGTGCTAGCTGTCCTTCGGGAAGCTAGCAAGGCTGCCATAAAAGAAAAGGCTCAGAGCCACGTGTGTCTGTGTTCAGAGAACTTGAACATAA
AAACCTCCCAAGTGAGATTAGGTAGTGTGAACTCTAACAGCAATTCAACCCGGATTAAGGAAATGCTGTACTTTTGGAGACAGTTGTAATAGAT
TTGTCTACCAAACATACAGAACACATGCCAAAGGTGGCATTTAAAAAATAAGAGATGAGAGAGGAAGAGGAGGCCACAGCAGCAATGAAAAGAA
AGCCTAGCCAACGACGGAGCCCAGTTCAGAGTCAGAGCACTTGCCACGTGAGAACAGCAAATCCTTTCTCATCTTACCAGACTGAGAAAGCGAA
ACACTCTGGCTAATAGAAAGGTGGAGCTGGGTTCCAGGCTCTCCAGTGGGCAGCACTGACGGTGGCCCATCCAGTACATCCTGCCCTGCTCCTG
CACGCATGAGTGTATGATGTCCGGTTCTGCCTGGCCCTGCATCTGGACAGTACCTGGAAGCAGACTTCCTGGACTTGGAGATTCAGTCAGTGTT
TCTGTTTCACAGCCTCCAGGCAGTTCCCCAGTCCCATGAGCCTGTCCTCTCTATGCCACTGGCCAGCCTGCCATCTGTGCCTTGGGCAAGCTGC
CTCCTCAGGTTTTTGAAGAGTTGTTGATTTGTTTGTAAGTTTGCATTTTGGTCCAGGTGCCCCTCACAGGACCCGCCCTTAATATGTGGTGTTG
CCTGGATCACTGGGTGACCTGACCCTCTGGAGATGGGCTTGTGGCAAGTCCTGTGCTCTGTCAGCTCTTTCTTTCCATTGTAAGGCTTCAGACT
TAAACTTCCCACAATGCAAAGAATAATGAATGGCTACTTTTAAAAGGTGTTGGTCTTATGCTCTCCTAGCTGAAAGTAATGAATGTGGTCTCTT
CTCTATGCAAACAAACCTTGTTCTTGTTATACAATCTACCCACTTCAGTTTAAGGAATTCATCTTAAAAATAAACTCTTACAAGTAGACAGAGC
ATGTAAGCACATGCTCACTGTGGTGTTGTCATTAGTACTTAAAAAACTATGGTGTGTGCACAGACCCGAAGCCTCGGCTCTTAAAAAGGAGGAG
GATATGAATATGCAGGTATGGAAAGAAATCTGAAGTGTATATCAAAATGGGAAAGATACAAAGTATGATTCCATGTGGAAATATAAATTGCACA
CACGTGTGCACACATTCTGCATGCTTCCATGAACAGTTACAGTGATTCTAGAGTGTAGATTGACCTGGACTTCCCCCAAAGCTGAAGGTTAGAA
GAAATCCTGGCGTGCTGAGGGGGGAGGGGAAGACATTGTTTTTTTCTTGTACTCTGTATAGTTATTTAACTTCCTTTTCTTTTGTGTTTCTTAA
GCAAATCTGTTAAATGAAAAGATGCTGTTTAGCTTGTTGTATTTAGCTTCCCTTGCCATCGCCCAAAGCCCAGGAAAATGGTGCTAAACATAAT
TAAAATAGGGTGATGATCAGGCTCTGCTTTTGAAAGACCTCACCTACGGGCTCACCCCTTACAGCAGGATTGTTTGGAATATCACAGAAACAGG
CATGTGGAGAAACTCCAAGGGCAAGAAGAATCCCATCTGCTTGTCTTTGCTGCTTTCACCTCAGATGAGTTCTCAGTTTTTATGTTTCAAGATG
CACTCAGTGGTCCACACCCTTGGGCCACATGCCGCCTACTGTCTGACGTCCTAATACCAAAACATTAGGCTGGAGATGAGACCAACCCAGATGA
CTATCAGTGAAAAAATGGGAAAAGTGTGGTATGTGTCTATAACAGAATATTATTTAGTTATGAATGTTGGAATTGATCATTAAGGACAACATGG
ATAGACTCCAAGAGCATTGAGTTAAGTGAAAGAGCCCAGGTTCTAAAAACCAAAGGCAGGCACACACTCTGCTTCTTCTGAATTTGTTCTTATA
AATGAATGGTCTGGGTTGGTGGATTTGCCACCCACACATATTCAAGGGTTGCACACTATATGTATGACCGAAATCTCAAACATCACACTGTACC
TCATAAACATGAGCAATTATTGGACGTCAATAACAAAATTTAAAACAATAAAGATACAACTTCCCCCAGCCTCGAAAACCACCTCTAAAATGTT
AACTATGATGGGTAGTCAGTATTTTCATTTTTCCATTTGATCCCAAGACTTCCAAAAACTTTAGTTTCTTGAACTTCTTTTTCTTGCCCTTAAC
ATTATGCTGGATGGTGTGTATTGTAGGTTGTTCTCAAGGAGAGGACTGTTGAGGGCAGGGACAGCAACACAGCCTGAAAAAACCCTTCACTTTC
ATTTTTTGTCTGAAAATATTTGGTCTGAAATTTTCTCTGTATGCCCATAGCAAAGGAATGACACCCCTCACCATGATACAGCCGCTGCATTCTG
TTCAGCCTTGAACATCCTGAACGATGATTAGAGCATCATAGAGTGTGTCACACTTAGGAAAGAATTACATATTGCTTTTGCACTCCTTTAAATT
TGATAACCTCCCAATTCAAAGCAATGCTTCCTCCTTTGTCTGCTAAGACTGGGAAGAAAGTAGGAAGGGTTCATGTAGGAAGGTTGACTTGTTA
TTTTGTTTTGTTTTGTTTTTCTTTTTTTCTTGTTTTGTTTCTCTTGCATCCCTGGGGATGGAACTCAGGACTTTATTCATGCAAGACAGGCCAC
TGAGCTATAATCCTAGCCTGGGAGTTAGCTTTCTTCATAAGAAATTGAGGGATATGTTACTGAGTCATTTCTTTTTCATAAGATGTTCAAACCT
AGCAGTTTCATAGGATTCCCCCTTAACATGTGCAATACCTTAGAAACACATTCCTTACTACACCCCTCTCTTCAAGGCCCTTGCATTCTTCTTC
CCTGGGTCTACTCTAAATATGGACTGTGCTCTAATTGGTGTCACCTACAGCATCAATCCTAGAGGTTGATGGTCATTGCAGATCATGCTCACTG
ACTTGAGTCTACGAATTGTGGAAAGCTTTCTGATAGAAACTCGCAGCTGTGTCCAGCCTTATTACACTGTCATCTATCAAGACCACACTAAGGT
GCTTCCTTTGGAGGTCCTTAACTCACTTTCTGATGGAAGCTCATTTCCTGGTCTTCCCTTCATCAGAGTTTTCACGTTACTCATGCGTTTTTGT
TTGCCTTCTAAAGAAAGAGGCAGTCTATGGGAAGAACCTTCTGGGAGTTGAAAGACCAACTCCACACCCATTTCTGATTGCAGCTGTGCCACCC
AAGCCAATGCTCTTGCCTCCTAAACCATGGTGTGTCTCCTATAGCAGCAGGAACTCTGGCCAGTGAATCTCTGAGGCTCCTCTGGCTCATATCA
CCTGAACTCTGTATTTTATATTTTCTTAATGCTGTTTGTAATGTGTTTGTTGGAACGTGCACACAAGGAGTACACCCTGTAGACATACATAATC
TGTGTAGGGAAGAGGAGCCTCATATATATTAAATAACAAAAGTATTCTACCTAGTTAATCATAGTAAATCACATTGATTGAGCACTTGGAACTA
CCCAAATAACATCCTAAGCATTTTCTCATTTTTACCTTATAAAAACCCACTGAAGGAGGATGTAGTTACCTCCAAGTTGCAGAAAGCTAAGACT
CAGCCTATAAGAAATTGACAGGAAGTCCCATAGGGAGCCAGACAGTTATGAGCCTACTTCAGCCCTAGGCTAGAAGCAACTTACTCTCCTCAAG
TGGTGGCTCTGTAGTGAATACATCTATTACCCTGGTATTACTCTCCCTTAAGCCCCTCTGTATCTTACAGATGCATGCCTTTGGGAGAGATTTC
TAGAGATTTGCTGGGTAGCTGGGCAAATCATAATACACTTAAGAACTCATTTACTTTTAGTTTCCGATTAAGGGCACAATTTCTTTAATTTGAC
CACAAACTTCTCAAAGACATACTTGACACACAGGCAACAGGCAAGTTAAGTATAAATTGGTGTGATTATAATTTCACCAGAAGTGTTGTTTTCT
AAGTAAGTGAAATTTTGCTTGCCTCTTCAGAGTCATTCCATTTGACTTATTAAGCATTTGCTGTCTTCCTTTCTCCACCCTATTGCTTTTGCTA
CCTGGTAAAGGTACCACATAGCCTATGCCTCTGTGGTGTACTACAGGTTACCAAGAGTTTACTTAACAAGTTGGTGTTCATGCTTGTGATTAAT
TTTCATCATGACCTGCTGAACTATGCCCAAATCATGGATTCTAGCCTCTTGTTATACCTGAGGAGAGAATTCTAAAGTAGTGGCCATCGCTCCT
TCCCATCAGCTCTCCAATTGCTTTAATCAGACCTATAACCCAGGTTAATCATTGTATTCATAATTGGGGTGTGTTGGCTTTACCTAGCACTTGG
GGGTTTTTTTGTTTTGTTTTGTTTTTTTAAGTAATGATTCAACTGGTTAGTGGGGCAAAAGCCAATATGCACAATAACTTGGTCTATCAGAAAG
GTTTTCATGAGATGGTAAATTTTCTGCAGGAAGACCTCCCAGCAGTAAAAGAATGCAAAGATCCTTGGTCATCAAGCCCCCCTGCCCCCAGAGT
GGTGGATGTCTCTGCAACAGAACTCACACTGGGGGCTTTTAGGCAAATCAGAGGACCTCTTAAAAACCATCAGTCAGCCGTGGTGACGCAGGGT
TGAATTAATGCCACGCTCTTGGCTGCCATGTTAATGCCATGTTCCTTCAGGTCTGATTTTGTACTCTATACCCCTATACACATGGGGACTGACC
TGTTGTTCCTCCTTCTCCCTGTGTGGTTTCATCTGCTCTGAGAGTTCCTATTGATCTGTCTGACCCACACCGTTTTAGAATCACAATCCCTACT
GTTTTCATTACACCGTGTCCTTCATGTAGGAAAAGTACCAGGATATAAGATGGGAATAGAGGTGAGATGGCCTTTTAATGTACAACTTCAGCCA
GCTGTGATTCTTAAGAAGTCCAGCTGTTACTATGAACATCATCCTTAAGATAGAGTTGAGTTTTGTCCTTTCAAAGATGGATCAGCCACACTTT
TAAAACTGAGTAACCACCATGTGTCTTTTTGCATGCAATTAACTAATCATGAAATGGACTGAGAGTGTGGCCTGCTTTTATTGGCTAATCCTAG
ATCTTTATCACACATACCCCTCCCTCAAGCTCTAGTGTGGCATGGCATGTCCACAGGCCCAGAAGTATCATGGATGATAATATCAAAGTTATTA
TATGGAAGACCATCACAACAAAACAAGTCAAAGCAGTTCCATTTCCCCGTCTCCAATTCCCACTCAATATTAATGCCAGCTGGTGAATCACGGG
TCCCCATGGCTCCACTCTCCGTCTCATGTACCAGCCTCTTAGGCTATGGCGTTGTGGCCAGGGGCCCCACTCCCAATCTCATGGAGGTTCTTTT
TATAATTATTTCTTTTTTTTTTTTTTGAGGTTATAGCTGTCATTTTCCTCTCCCCTCTGCTCTTTGGGGGTTATTTACTGGCCTTTCAAAAGGC
AAGTGGTGTGGGTATCTGCTAACCAGCCTGCCACTTCCCCACTTCAATGTCTCTACATTATAAAGAATATCTGTTTAACCAGATTTCTGTGTGC
TTATTGTCTTTGAATACCTCCCTAACATCCCCAAACTTTGAAAACCTTCATTCTGGGCCATGACTCTGTATGACTGAAGCTGTCAAATTGTTTG
TATACCTTCTGCATGCTCCTGGATAGAGGCTTTCCTTGCTTATTGCAAAGATGAGAAGCAGAGCCACAAGTCCTGCCACTGTTACTGTAAATGT
CTTCTATTTAGGGTGAGGGAGACAGTGCTGACTACTGCTCCAAGATTCTAGCACAATGTGAACACTTTTAATCAGCCTCTTGGGCTTTTTGTTT
TTCTTTGGGAAATTATTATAATCTTACCCCATCTTTAACTATTTATCTCGTATCAGCTATCCAAATATACCCAATAATGCTCTTTCTGAAAATA
TGCCAGTTGCTCTAATCAAGCTAACCTGGGATAGTACATTGTGATGTCTCTTTCTTAGCGATTGTAACACTATTCCCCAAAGCATGTCTTTGGT
GCAGATTCTATTTTAAAGATTCCAGTAAAGCTTGTCTCATGTACTTATTTTAATGATCTTTTCTGGCTCCTAAAAAATGTTGGTGCAGTTCTTG
TGAGTTGATCCAATTTTCATACCATCTCTGTTCGTGTTCTCAGAAAATTTTACTCATTGGCATGTTGTTTACATAATGCTCACAAAAGTCTGGG
TTAGTGTTTGTTGGGAGGTCATTTCGCCTATTGGGCCAAGTTGCCAACTCAACGGGCACACGTCTGGTAGCCTACCTTCAGGATCTTGAGCCAG
ACTTTCTTTTGTAAGTAGCTACAATGAGTCAGTCAGCATAGATTTAGGGCCTCACTGGCTGTTTCTCCTATTCAGTTTGAGCCTGTCAATCCCT
TTAAATCAGCTCCAGAAAGGTGCACACTTTCAGAGCTCCAGCCTTGGTGAGAAGAGAGCGTTTTCTACCAGCGACAGCTTCACTGTGTCGATCT
CAATTTCTCCATCCTGGAGCTAGACAAACTTCATTCTAACCCAGCCAATGATTGCTAGGGAGATGGGATTAGGAGGGGAGTGAGGGGGTAACTG
GAAGTGAAAGTGGCCAAAAAGCTGTCTACACGTGTATGAGATTATCAAATAATAACACAGAAGGGAAGGAGGCAGAGGGAAGGACCAAAAAAAG
CTGTATATGCATGTGTGAGGTTTTCAAATAATAGCAAAGAAGGAAGAACGGGAGAGGAAGGGAGAATGGGAACCCTACTTTATGATGTGTAACA
AACTTTTATCTTTGTATGCTTTTGAGTTTGTCAAAGCACGAGAAGAGCCTCAGAAATACAGCCACCATTAGCCCAGCAGTAACATTTCCAAAGA
TGTATACAATGATGATCTTTAAGGATATGTCCAAAATTGACTGAAAGGAGGTTCAGTGCAAAATTGATTATATAGACTCAAAACAATATATAGA
GTCAAAAGAAGCAAAACAATGAACCTAATTCCTTATTTCCCCTATAAATAAAATTGTAGAAATAGGTTTGTATCAACAAATTGTATTGAGGANN
NNNNNNNNNNNNNNNNNNTATATCAATATTTATGTAGTAATTTGGTATCAGGAATAGTTAAGGCCACAGATGTTGTTTTGCTTTTTGTTGTTTT
CCTTTTGTAAATCTTTGTTCTGTCCTCTGATGTCTATTTAGTGCTTAGCAGTGATATTCATTCCTAGGAAGAGAAATATGTATTTGTCCTAACA
GGGAAGCATTAATTCAGAGCAGGTACCCCTTCAGTCTGCCCTAGTGCTGTCAAGGTGAAGCCCAGGCAGTGAGGTTTTCCCAGCAGCTGACAAC
GCCTGCCAGAAACTCAAGCTCTGATTTGTGTCTGCAGGAAAGGAGAGCCCCCCAGTCAGTGACACTCCAGATGAAGGGGATGAGCCCATGCCTG
TCCCTGAGGACCTGTCCACTACCTCTCGAGCACAGCAGAACTCCAAGAGTGATCGAGGCATGGGTAAGTGGCTCACCCTGCTGTCCTGCAGGCT
GTAGCATATGCACAGTTAGGAAATGGCTGCTGTGTAGGATAAGGTAGGTGGGCTCATTCCCTCTTGTCCTCTGAGGTCAGTCACATAACAGGGT
TTCCTCAAGTTGGGATATGGGTGATCTGCTTTAATTGATGTGTTTATTTTTAAGGTGGTTTTCTGTTGACTACAGGTGATCTTGGTGTTACTAT
GTGCTGAACAACATGGAACTGTATGAAATACATTTGTATTAATTTAAAGGTCACTTTAAATCAAGGAGTTCGGCCAGTGTTGGAGCATATAGAT
CTCCTTTGCTTAGTCCTGAAAACACCCTGTATGCTAGGGGTTCTTTTCAAAACATAAACAGATGGTCACAACCTGCCTGTGGTTCTCATCACCA
AGCGAGAGGCTCATTCTGGGAAACAGGAGAAATTGTCCTTTGACCATGCCACACAGGGAGAGCCCCTACTTCACAGTCAGGCTAAGGACTTAGT
TCTTGTGGAGAAAGAGGTGGGCTTGCCCAGAATTTCCACAGCTGCTGTGGAACCTCTGCAGAATCATCATGCTGTAGGTGTGATTTAAGGAACC
GAAAACCAGGCTGGTTCCTGGAGAGCCCGAAAGATGTGTGTATTTCACGTGTCATAGATGAATAGGTCACAAGTGCTGTGGGCACCTTGTCCCT
TTGGCCGTGTAGTGTGAGCAGGGAGCAGGCTTCGCTGGCACTGCTGGCTGGCTCCCTCACGGGCCTTTGCATCCTAAATCAAGTGCTCAGTTCC
AGGTGCCCTTGGTATGCGCACTCTGTAGAACATGTCATTCCCAGGGTCTTCTCTCCCTGACTGTCACCATGTACTATTTTCAGTTCAATTTCCT
TTCAAGATGAAGTTTAGAAAGTAATCATTTCAAACTTGAAAAATTAAAATGCCTTGGTAAACGTTTTGCCTGATTTGGCAGTAGCAGTTGACTT
GGCCCTGAAGACAGGAGGAGGCTATTTGATGTGAGCCTCAGAAACAAAGAGAGCAAAGTTTTTGGTGGACAACTAGAAGCCACAACATCACCTG
CTAGAAAAACTGAGAGCAAAAGCCCAGAGAACAGATAGTACATGGGAAAGGGATGAGAAAACCCACCATTGGAACACCCCCAGCCACACTACAT
GGTGGCCGCCTTGTCCTGTGTGATTGTTGAAGGAAATCCACCCATTTCCACATGTAGGCTATATATAGCCTGTAAGTTTCATAATGTGTAGAAT
ATAACATTAAATAGAATGGGGTAATATCTAGGAGGTATTCACAAATTACAGGAAATAAAGTTGAAGCAATACTTCACCACACTGCCTTATGGAG
TGGCTCATGAGAATCATGGATTAAAAAGGACAGGGGACCAGGAATCAGGAGAGTTTGGAATAGGATCAAAGGGCAACAGGGTCAAGCAAGAGTC
ACCATGGAAACAGGGAGGCAGGAGGGCAAGTCTCAGAAGGGACACATCCAAGATTCTCGTCCCTGACTGTGACCCTCAGGGCAGAACTTCTCAT
TTCTCTGAGCCTCCATTTTCCTCATGTGAGCGAGAGCACTACCGCATGCCCCAGAGTGGCTTTGTGGGCCAGATGAATAAAGCAGAAGACAGAA
TGGCAGACAGACTAAAAGAGAGAGAAAGTGTGTGGGTGCATCGCAGGCACAAATGTTCCTAGGGACTGTTCCTAGAGAAAAGGGATCCATGCGG
CCTCAGTAGTAAGATGAGGACAGACAACAGAGCATAAGGAAGAGATGAGTCCATTAATAAAAAACGGACCCATAAAACACGCAAAATTGAAAAC
AGCCAGGAGAGAGAGAGAAATGTGTTAGAGAAGGACAGCGACAAGAGGAGACTGACTGCAGATTGAGAGCTCAAGCCTGTGGAGTGACCAGCAG
TGAGGACGCAACAACCTGGGTCACTCAGTGGAGAGAAATGGGCCTGCTACTGACAGCTGTGACCATGCCAGGAGACTCTGTACCAGACAAGAAC
AGACAGAGTTAGAAAACAAGTGGTGAGAGACTGGAAACCAGCCCCTGACTGGCTAGTGGGTCATTCCCAGGATTAGAAAAGTTGGGGTTTCACA
CACTGCTGCTTTCTGTAATAATGCTGAAAGGACACCATAGATCTATGTATAAAAATTGGGACAATGGCAGTCAGAGGCAGAGTAATACAAGCAC
CATTATTGGGCCTCCATTTTCTTCAAAGTCTGAATAAGAAGAAAAAAAGATTTGAAGAGCTGCTTGAAGAATTTAAGTCAGACCCGAAGAAGAA
CTTTCTGGAAAGCAGGGATTTAGTCATCGGAACGGTCTACTTAATGGCACTGTGGCTTGTTCTTCTGAAGTTATCTCTTTAATAATGAGAACTC
TCAGCTCTCTGAAGTGGTTGTGTCTGAAACCATTTGAAGCTGACGAAGCACTCAATTGACCTCTTCTGGCAAACCTGAGTTTACCAAGTCTCAG
ATGTTTGCTGGAAGAAGGGCAGAGCCGATTGCACTGACACAGAACTCTAGGGCCTCATAAAGGCCTTCCTGTGGGACAGTGTCCAGGTTGATGT
CTAGAGAGCAGATGGGCAACTATAAAGAAAAGTGATGGCTCAATACTCTATTCCTTTGCTCAGTAATACATCTTCCTTCTTTCTGGTGAGTTTC
CCCCTGGTTTGGGTTTGCTCTTGTCACTTGTGGCCTAGAAGAGAACAGACCTGTGCTCTCCTTCCCAGCTGGCAAGCTGGGGATTGTGGCCAAA
CTGTGATAAAATCCAGAGCACCACTCTGTTTGAATGTGACCATAGGGAAGCTGGGACAGAGAGAGAAGGGAACCCAGAATGAAAGGTGGAATTC
CAGGCTAAATGCACAAACACCTAGAAAAAGCCAAGCCAGTATGAGGCTTGGCAGAGTCCAAGGGAAAAGCCAATAGAAGGATAGGCCTGCTCAA
GGCTGAGCTGAAAATGGGATGTTGCTAAGCAGGGGAGGCTAAGTCCATGACTGAGCACTGGACAATCTGTGCAGAACCCAGACAGAAGGAAATG
GGATTGAACTCTGAGAAGGTGCATTCTAGGATATGCACCCTTCAGTCTCCCTTTGAGTGTATCTCAATCTGATATTCATCTGCCAGATCTGAGA
GAGGCTGACCCTGTACAGCCTCTCAGTGAGTGCTTGCACCTAACTTTTACTCTTCTTAGCTTAGATGAGAAGAGAGAAAACAGATACAAAAGAA
AATCTACATTTTTCATTCTTCTAGGAATCCTGAAATAGTGAAGTGTATAAAAATATTGATCTTCGTTTTTATTTTGTTTGGTTGTCTCCTCCTC
CTCCTCCTCCTCTTCCTCTTCCTCCTCCTCTTCTTCCTCCTCCTTCCCCTCCTCCCCCTCCTCTTCCTCCTCCCCCTCTTCCTGTTCTTTTTTC
TCCTCCTCTTCCTCCTCTTTCCTTCTCCTCCTGCTCTTTCTCCTCCTTCATTTCTTCTTCTTCTTCCTCCTCCTCCTCCTTCTTCTGTATTCAT
TACATTGACTCAGCCATGAGGGCAGGGCTGAAGCAGGAACTATAAGCAATCATTTTAAGTTCTCATTGTATATAGGCAATGTACATGTTACTGT
GTCACATGAGAACATTTAGAAATATATTGTCACATATTGACAACTAATTGTCCTGTGCCAGTGTCCTCTCCCATGTGTATGTGTGGGGGTAGCA
ATAAGTTTTGGTTGGCATACAAAATCTGGGATTCACTGTATCATTTTTAAACATACTATGGCACCAGGCTTATTCGCCCCATCACTACCATCAA
CTTTAGGCAGTCAAGGATGGGTCCTCAGGATATAGAGCAGCCATGAGATTCTAGGCCTAGGAGAAAGAAGGGCTGGAGGGAGGTTTTTAGGACT
GAAAGAGAGACTGCAAGCCTGGCTGGCGAGTGGGAAGCTCGCTTCAAGTACGGATGTCAGCCCAGTTATCTAATCAGCAGCCAAAGGAAGTTGT
TTTTATCAGTGAAATGCATCTCTTCTGAAGCGACAGCCCCACTTCAGCCTAGAAGCAGCATTATCTGGCAGCATCTGAGCAGCTGCAGCAAGTG
CAGGGACTGGAGGTGCAAGTAGGCAGGATCTCCAGCCTCGGGAGCTGCCACCAGCACTGAGTTCCTCTTTTCTGGAGGTGACTGAAGTATGTAG
GTTGGCTCCGTGTGTGCCTTGATTTCTCTTTCTCCCTTCCTTGCCCCGCTTCAGAGCTGAACGTAGCAGAGCACATCTGTCAAATTCAATACTG
TTCATAATGTAACTTACTATCTCTGACCTGAAATGCTTGTAAAAGCCACTGGTACTCCACCATAGGCATTTCTAAGTCAGCTCAGAAGTATTCC
TTTTTCATGGATTTCTCAAAACATGAGACAGTAAGCAGTGGTATAAAAACTATTTTCCCACCCGCCCTCTCTTTCCATCAGAGGAATGAGAATT
GCAGGCTGCCACCCAGGTCTTTGGCCTTCTGCATGCATCTCCCTTACTCTGTCAACCAGTAGGAATCACAAAGATCTTCACAGACCATCCTAAC
AGTTGGTTATTCCTGAACGTTGAAGGGACTCTGCATATCACCTGTCATCATGGATTGGGCTTGGGTGTACACAGGGAGACCAAGCAGCAGGAAT
TTAAATCCATTCCTTGGAGGAAACAGAAACTCAAATGTGTCAGTCAGCACCTGTGTGTGCATCCTTGCTTCTGTGTATGTGGGCATTATGTGTT
GTGTGTATGATGGATGAATGCATTTATGTGCATGCTAGATGTGTATGTGCTAGTTTATGTGTTCCTGTGGGTGGCATGTGCATATATGTGTATA
TTTGTGCACATGTATAGTATATGTCCATATAAATATGTAATATTTTATGCATGTGTGTTTGATATATAAATATGTGTAGTGTATTAGGGTATGA
ATGTGTGAGGTATGTAAACAATGTATATGCATATGTTGTGCTGTGTATTTATGATGTGTTTGTACATGTAATATATACGTATGGTATATGGTAT
GTGCTATGTGTAGTATATATGTATATCTGTATGGGATGTGTATTTGTCATAGTATGTGTGTGGTATATCTATGATATTTTATGTATCACAGAGT
ATATGGGTATGGCAAGTTTATAGTGTGTATCTGTGATGAGTATGAGTGTTTGTATGATGTGTAGCAGTGTTTGAGTAGTGTGTCTGGTATATAT
TTGGTATCTGTGACTTGTGTTATATGTATGTTTGTACCATGTGTGTGGTATGTATATATGTGGCATGTATGTGTGTGGCTTGTATGTGGTATGT
GTGATTTGTGTAATATATATGTAATATTATATATGTCTGTGTCTTGTGTGTAGTGCTTATGTATGGAGTATATATGTGGTGTATATATGTTTGG
TATGTGTTATATGTGTATGGTGTGCAGGGAGCTTGTATGTGCTGTGTATGTATGTGGTATTCATGTGTGTAGTTGAACAATTTGTGTAGTACTA
TGTTGTACACACTGTGACATGTGTGGTGATATGTATATGTGTTATGTGTTATGAGATAAACGTGTGGTTGTTGCTTTTTCTACACTTACAGAAA
TTATGTTAGGGTATATGAAAGTACACGCTGCATGCCGTGGGAGCATGTATAGACATTAAAATACAACTCACAAAAGTCAGTTCTCTTTGTCCAC
CAGTGGGTTCCATGGACTGAAGTCTGGCCATTAAGCTTTGCAGAAAGCACCTTTACCGACTAAGCTATCTTACTAGCTCAAATGTGTGGATCTT
GACAGTATGCCATGGCTATTTTCTTGGAGATTCTGGCTTTCAACTAGATGCTTTAGAAATAGTTAGAAGCCCTGGTTCTGGTGTGTCCTAAGAT
CATCAGTGCTGCGGTGACTGCCATCTCTGCACCTAGGAGATGAGGGGTTCGCCTTACCTAAGCAATACATGCACATTATACTCTGACATGGAGT
TCACCCCAGCCAATATCCATGATGTTGTTAATGGCATAATATTAATGTCAATAACTCGTGTTGTGCCAATGACAAAAATTACATATCATCTCAG
TTGGGCAGCCCTTTGCCTGAGATTCCATTTGCAAGTGACATGGGGCAGAAGCCTGGACACTAAGCCTGACATGAAATCACACCCTGGGTCCCAG
AGAGGACAACTCCAGGTCTCTGAAAGAACTTCACTACCTTCCTGTCTGAACCATCAACTAGAACTCCTCCCCATAAGAGGCCTAAGCCAGCTCT
GCCAGGCCCTGGCAGAGAGAGAATGGAGACAAATAAGCTAATGTATGGTTGGGAAGAACATGGCTTTCCACTTTGGGCTAGCCTCTTCCTGTAC
ATAAAGGTCACTCTACAACCCACTTAGTGCCTTGTATTTGTAGACTGCTGACCATGAAATCCTGAAATTTCTTTAGTATCTGACCCAAAACAGA
ACCACTTACATGCAAAGCACAACCCTCCCCCAACCCGTGCTGAATTCATTTTAGTATTTGTGTACATGTTGTTTGGTCAGCTCCCAGCTTTGTT
TAGGATGTCTGGACCTCAGAGATAGAAACTCATCCCAGAAACCTTTAGTGGATAAGATGGACTCCCACTGCCTTGGAAACCTTGACAACTAAAG
AAAGCTGTAGTGCCTGTGTTACAACGACACAACGATAAACTCACAAGAATATGAGCTTTTTGTAGAGAATATATTCAAAGAATAAATAGTGTGT
CCTAACTGGGCTGAGGATTGGGGTTCACGAGGGGTTCAGATTCATGCAGTAGCTGGTATGCTCAGCAGTATGAGAGGGAGCCAGGCCTATTTTA
ACTGTAGAATGAGTTGAGCAAAGAGGGAAGAATGCTTAAAAGCAAAAGAGTAGAGGCACAAGAGGACCCCAGCATCTTGATCTGGGGTGTGAGC
AGTGGTGCAGGCAAGGGGCCAGGTGATACCTCCAGCTGCTTTACATAGTAGACTTTGTTCTCACGATAGCAACTGTTAGGAGTCTTGGTCTCAG
AGAAGGGTATTTGAACCTGTGCCAAGCAGTGTGAGTAAGGAGAAGTCAGGGAGAGATGGGGTTTGTCTACTGTCTTGGTATAGGTCCATGGGTA
TAGTGACCAACTACAGTGATTGTGGCAACAATGATAGTAATCCAGCGCTGATTAGCATAGAATCATGATAATTCTGATCCAGGGAATTGGCTGG
AATGTAGGGATGGAAGAGGCAGATGTTCCATCTATGCATAGAGTCAGCTAGGTGAGACAAGAGAGGGAACCCTTGGAGGAACACTAGCCTTTAA
CTGTAGAGAAGAGGGGTAGAGGGGAGGAGACAGTCCTGCAGTTCAGATGAAATTTCTCAGAAGAATTATAGGATGGAAGCCACAGAAAACACAT
GGGACAAAAGTGAGAAAGGTCAAAGAGAACTTAGTGAGTGCCCTTACTGAGGAGAATTTGGGCCAGTTGTGCATTTCCCAAAAGTAGCTCAACG
AGTTGCACGCAGAAACCAGACTGCCACGGAAACCAGACTGATGATCAGGGAATGGGAGGGTAGCCATGAGGGAGGATGGACAAGTCAGATGGCA
TGGGAACATGAGCTGAGAGTTGCTGGCTGAAGGGGAGATTTCCAGGCTGATACTTTAGGATGAGAATAGTTAAGAAGTAGGAAGGAGGCCAGGC
GTGGTGACACATGCCTTTAACTGCAGAAGCTGGAGGATCTCTCAGTTGCAGGCCAGCCTGGCCTACACAGGGATTTCCAGGACAGCCGGGGCTA
CACAGAGAAACCCTGTCAAAAACTAAAATTACGGGAAATAGGAAGGACTGGAGAAAAATAGACTAAGCTAAGAAGAAACCTGGGAAGCAAGCAG
GGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNCCCAGCAGTATGGATGGAATGATGCGTGTTGCCTTATTGGGGATGAGA
AAGGGAGAGGAGCTATCAAGCAAGAGTTTTTCTAAGAGGAAACATGGCAAGATCTGATGCTCAGCACCTCAACTGTTTTAGGATACTGATTTCA
CAGTAGCACAGCTGTTGTTCAGGGCAGGGATACAGAGCCTTTGTGGGAGGCTGTCCCTGCCACACTGACCAGTAGTCCTACCCCACCCAGGTCT
CTGTGATACCCACATGTCCCCATACAGCTCCCAGGTGAGAAGCTGACTCAGATGTCAGCCACACTTCCCTCAGCGTTCCCACACTCGAGAGCTT
TCGAGTTCACACAGGACATCTTCAGCTGCCTAAGACTTTTATTGTGGTCACTAATGTTCCAGAACCCAACATAGAAATTAAAAGTATACTATAG
AGAATTTGGACCAAAAAAGATGCCCAAGCCCCACTCTCAAATGTGCAGCCCTTCTTCCGGCTTTTTTTCTCTTTGTGTCTCCAAAAATACAGGT
TAACTGTGCAAAGCGGTTTGTTTCTGTAAATAAAACCCAGCATTAATCTTATCTTCACTGTACTCTGACCACTGAAATTCAAAAATCGCATTGT
CCTCCCTTCATTTACTCAGAGAAACAAAGAGGAGTCCAAGATTAAAAGGCCCTGGAACAGATAGACAAACGCTACTTTCTGTTTCTGGGTTTGG
TCCCAGTGTTATTTTCTGCTTTGTTCTGTGTGTGTTGAAAGAGTCAGTGGCTCTTCTGTATGTAGAGCAGTCAACTGACATTCCGTTAACTTTC
CTTCTGCATGGGGTTTCAGAAGAATGGAAAAAAAAAAAAAAACAGGTTTTGTGCTGTGGTTGCCAGTTGCCACCTGTCCTCCTATCTGGTTAGG
ACATTTTTCTGATTTGGGAAGTATAAATATAACCCAGAAAAAAAAAAAAAAAACAGCTTCCCTTCCTTTTATGCAAAAGTAGAAAGGTTTAAGT
AGAGACACCAGGCTCTACCCATCACCCTTCAAGTTTGCACAGAAGCAGCTAAGCCCTGTGCAGATGCCTGCTGGGGGAGGGGAAGTGAGAAGCG
TCCCTCCTCAGGCCCTGAGTGGTTGAGTTCCAGCTTCTGACCTCTACCAGACGAGCTCAACAGAGCACAGATCTGCTCAGAATCTCAAACCTTT
TGTTGGGCTTTATTTATTTATTTATTTATTTATTTATTTATCTATCTATTTATTTATTTTGATATGAGAAACTTAGAAATAATTCAAAATGTAT
GCTCAGTTGGCAGGGAGTCAGCTGACCTCAATAGATCCATTCATGCTGCCAGACTCTTAATGAACAGCTTGAAGACCACCATGGTTCGAACTAT
TGACACCACAGATACTTGGCCAATGCTACAAGTTAGGGCTTTTTTCGTTTTGTTTTAATTTGGTTTGGGATTTGAGAGCTGGCTTGATACAATT
TTCATATCATGACACGGAAGTGTGGGGGGGTATTTTTTGGATAGCTATTTGTGCAGTAAAGGCCAATAATTGGCTCCCAGTCGCTCTCTGCCAT
TGGTATCACAAATGAGGCCTCCTGCAGCAACTGCCCTCTCCCTTGTCATGAAGAGAGAGGACTTGTGTTGAATCTGTCAGCTATGTTCCACCAG
GATGGCAATAGACATCATGAGGTGACTCCCCTCGCTGCTCCCTGAACCCATGGTGACAATTTCTCAGTAAGACTTAGCAATATGCAAATGTGGA
AGAGAATGATCAAAAGTTCACCTTACCATGGAGGGACCCCAGACTCTTGGACCCCCTGAAGCACCCCACTTGCTGGAACGCACTTCTGTGGTAA
AAAAGCCATTCATGCTACCTCTGTTAGGAGCAAGGTCAAGGCAGGCATCTCTGGCACAGGACTGGCGTGTGTCATTGGAAGGCTGGCTGCCAGG
AGAGTACTTCCCCTACAGGGGATGTGGCTGCAGGCTGTCGATTTCCACTGAAGTCACCAAGCCAGGATTCCAGTGAAGCACTGAACGTCCAGCA
TCTTCCCCATCCAGCAGGGTATGAGGTGTGATCATGTGATCCATGGATGGCATCCAGGCTGACTCCTGCTGCCTGGCAGGGCCCAGGCAATTGT
GGACATTGACTTTTTCCAAATGAGAGACCGTTACCCTAGAAGTGAAACCCTGCTGAAATGAGAAAAAGGCTTTTATTACAGCCCTGCAGGCAGC
CATATGATTATTGTTATTATTATTCTTAGAACCTGGCAAATGCTGAGTGCTTTCACAGTGTCTCCACACTCCACTTGAACTTGGTGGCAATAAA
AATAAGAATCAGCAGTGACCCCGGAGCATTTCTCTTGGCTTCTGTTGGCACCCCAATGTGTAATAATGACAATTAAGTATTGCCTGGTGTAGTG
ACCACTTGTTTGAGCCTAGTATGACAGTTGGAACTGCCTTCAGTGAGATCAGGGCTGGGGAGAGAAGGCTGACTTAAGTGCAGGCAGGGGTTTA
AATCCTAAGTCCTAAGAAGATGATGCTGGCTTGCCGTAAAGCCATGTGTGTTTCCTTACAGTTTTGGAAGACTGTTACTTCTCCGTGGGTGTTT
AGCCTAGACTTTGCACCTCTTGCGGGTTTCCGTTTCTCTGTGTGTTTCTTGTATTTCACTAACTACTTTTGTTATTGGTGGGATGCCTCTTAAG
TATTCACCCTTAGCTTAACCCGCACTGTGCCTACATACCACACGCATGCCCATACTCCCTGTCATCCCCTGGAGAGACAGACAGACACACAGGC
TCACCTTTAGCTCAAGACTTTGAAGCATGTCAACCAGGAGCAGTCCCTGTCTCGGTCAGGAGTCTCAGATGAGTGGTAGCCATTGTTTCTGGAA
TATAGCTATGCCATGCATGATTGCCTGTTTTAGTGGAGCACAGTCAGCTTGAAATGTAGTAATCTAGGTGCTAGATGCTTCTGAATTCCCTAGA
GAAAGGAATTATTTTTTTAAAGGAAAAAAATCCTTCAGGTAAAATTAATGTTAATATATTTTATTTAGTCAATATACCCAAATAGTATTACAAA
TATGTACTCACTGTACTTGTTGTCTTGTTTTTTGAAAACTTGTACTTTTCCCGTGGACTGAGTGTTTGAGTCCAGTGTTTGATGCTTATATCAC
ATCTCAGTTTGGGTTGAAGCCAGCTATTTGGAGGGGGCTGATTTTTTTTCCAGTGCTGCACTCAAACACTGAGCTGTGGGCCAAGCCTTTGGGC
AGGGATCTTGACAGTTCCTGAGGAAAGCATATAATTGCATTGTAGAGAATATAATGTGAATGCTTGCTGAGTTGTATCATCTAGTTCTTCCATA
TTTTCCTCAGTCTCAGTCCTGTCCCGTTTGTACTGTTGAGTTCCACTTGGCATCAGCATTTTAGCAGGGGCCCCACATGCATTTGCTTCCACCC
TAAGAGCAGTAGGAGCAGAAATACAGGAACCTGGAAAGCCCATCCTCTTCCCTGAGCCCAACTGACATGCGTGGGACAGGGGATGCTTCTGGCT
TTTGTTTGTATCGCTTCCGTCTATTCCTATGCTCTCCCGTGTATCACTGTGAGCCTATTCATTAAATGAAACATCTTCAAAACATGACAGTTGA
TTCATAAACAAGGCACATTACAGAAGAATACCACAGGCATCTGCTCCCACCAAACCCAGCCTCAGACACACAGTATGACTGCTGTGGAAATGGT
GGCCACTGAGTCCTGTTGCTGTTCCCTAAAAAAATAACACTGCTGCAACCTGATGAGGCAGCAGGGAGGAAGGGGTGTTGGAAAGGACTTTTAA
GGGAGAAATAGAGGCCAGCTTCCCTCCTGCAAGCTTAAAATGAAGCCTGGGATTTGCCTCACTACCCACTGTGGTTCTGAAGGCCCAGGGATAG
ATGGCTCTGGCATTCCCGCCTCTCCCACTTCACTCCCACCTGAGCCCAGCTTATACCCCCAGCCTTCGCAGACGTCTGCACGCCACTGCCCACT
GCACTTCTTACAGTTCAGGAGACAGGGTGTGTCCCTGTCCATGTGACTGCTGGGTTTCCTGAGCACGCCAGGATCCACAGAGATAGGATCTGTC
ATTGTGGCTTTCAATTGTTATTGTCTTATGATTCTCCCTCCCTACCTCTCCCCTCACCCTTCCCACCCTTCCACCCTAGTCTTTTCATTTTCTC
TCTCAGTCTTCTTNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNTCTTCTTTCTCCTCCTCTTCTTCCTCCTCATCTTCCTCTTCCTCATCTTCTTTCCTCCATTCCTTCAAACATTTGGTCATA
GATACATTGTCATCAAATTTTGGTTGCTTGCATGGATCAAAGGACCAATAGATGGGTTGATTATATTGTACTAGGCCACCCTCAGCACTGCTCA
AGAGGCACAGGCAGCTCGTCATGGATAGCCTGGCTGTCATAAGTAACCTGAAGCATCTTTAGGAGCCTATTGTCAGTAACTAGCAGAGTCAAAG
CCACATTTCTCAAGGCTTTTGTGTTTTGTGTTTTTTCTGTTTTGCTTCCTGTTAAATGAGTTAGTTGGTCATTCCCTGGATCAGGATGGATTGT
AATGAACTATGTCTAGCTGGTTGTATCACAGAAAGCTTACCACCCAGTGTCGATGGGTATGCAAGCAGGACTTCTGAGCGCATGAGAGTCTTGA
CTTTGTTGACTGCTCCAGGCTATAAACCAAGCTTTACTGTAGCAAAGTTTGAAGAAGTAGATTTGTTTTTTCCTGAATTGAGAAAACCAAAAGA
AAATCTGCATGATTACTTGACATAGTCCTCAAAGCTGTGTAGTCTTGACAATACTGCCATTGCGCCTACCTCTACCCTGGAGTGTCATGAGCAT
CACAGGTGGACATGAACCTGTTCCTTTAAAGAGTTACATGTACACCATGTCTTGCCTCTGAATCATCTTCAGATTTGGAGCTAAGGGGAGATGT
GTTCTGAGTGGCTCGAGCATTCTAGAGCTCAAGGGACAACTGAAGGTAGTATTACGAAAGACACTTGTCCAGGCACCCTGTTAGGCAGAAGCCA
CCAAGTAAGATGAGATCACAGAATTTACCCTTTGATAATATATATACATATATATACAGGGATAGAGAGTCAAGAAAATAACAAGAGAATGGTG
AGCAATAAAATATGGAAGAATATAGTTTGTGGTACAAACCACAAACCATTCTGTTGGTTTTAGACACTTAAAGTTCAATATTTATGAAATAGTC
AGCTAGGTGTAGTGAAGTTCATAGCTGCTTTTATCAAATTCTCTGATGGTAGTATGACTATTCTCCTGGGAAAGGCAAAAAATTGTCATGGGTT
AGAAAATGCCATTTGAGTCACCAGTGGACCACATGTGTCACAGTCCCATTGATGGTATCTGCTAGTCTATCATAGCTGTCTGCATCTGTTCTTA
ACCCGCTGCATCATCACATAGGACTGAGACCACCTCAGGGTGCATTTCTTCTTCAAGAATATCTATGTTTTTAAGTGATGCATTAAAGTAAGAG
CTTCTGTCCCAACATGGATGGAACAGACAGTGAATATACATCATTGGCTTTAATGTTAACGCTTTTCTCCCATGCCCCATACCCACAGAGGAAG
GCAAACACAAGCACAGCTTGTGAGCAATATCATGTTGCTGCTGTGTCAAAAGGTCCCTATTCCCATGAAATACACTGTGTAAAAGGCCACCCAG
ACCTCATCTGGACTCAGCAGTCCATTTAAAACCATCAGCAGGCAGGTCACTCGCTGTAGGTCCTGGCAGCATGGGGGGCCAGACATCAAGCACT
GAACTAAGTCTATGCATCTGCAGACCATTGCTCCTGTCTACAACATTTCCTCAGCAGCCAGAGCTCTTCCAGTTGGAGATGAGACAAACACTAA
ATACCCTTCCTTCTAATAATTCCTTTCCTTGGAATATAATAAAGACAAAGAGTGAAAAACTACAGTGTATTTGCCTTGCTTCTCACCATTCTCA
CTCTTTAGAGGTTTCTTGTCCATGTGCATTTCTCTTATATCAGTGTGGTGGCATGTCATAGCTAGCCATGAATCCAGAGTGTTCACAGATAATG
ATCTATGGGATCATGGCTTCTTAAGCACATGCTCATTGTTTATAAACTCTTAGAGTTCTGAGGACGGAAGGCTGAGTAGGAAGGCCACCTCCCT
CACAGTTGGTCTACTCTCACAAATGTGTGAGGGGTCTGCAGCCACATCCCTTGTTGCCTGCTGACCTGCAGCTCAGAAACAGTTTGTCAGAAGT
CTGAAGACACTCATAGATCTTTTGCATTCTGGAACCTTTGACATTTGGCTGAACAATGGGATCTGCTCATCTTCTTCTTGCTTGGTTTAATGTG
TCTCAGCTCAGTCTTTGGAGGTCTTTTGCAATTGCCAGACACATAGACATTGTAGAATAAGACAGGACTCCTGCCCTTAAGAGGTTCTTAGTTA
CTTCTTCGGAAGATGGCTGAAATATCCCTTGTTGCTGTTTTGATACCGTGATACTCTGTGGATAGAATGAGGGAATGATGAAGTTAGCATTGCT
GAGCTACTAAGGGTTCTGCATTGATCCCTGATGCCCTAGACAACACAGACACCTACAAGAGGAAACAGCTCATTGTCCAAGCTTACTGGCAAAG
TCATGTTTTCAAGCCATTCAAAATCCAGTTCTTTAAAGGGATCCAAAACAACTAATACACTTCAGTTAAAGGAGTAAAAACATCCAACTGCCCC
TGCACTGTTCAGTTCTCTTATTCCAGTGGAGGTTATTTTACAACGAGATTTCTCCATCAGGTCTTCTATTGAATTTCATTTTTACTTTGGCAAA
TTAATTTTCTCCTGAAATCCGTGTTTTCTGGAGAAAGGTTTGGTACCATGCCACATAAGTGTGTCAAATCACTCAAGAGCAGATGGTTTACTAT
CAATGCACAAAGAGTTCTTAGAAAGGTTAAGTGAAGGGGAACCAGGTAGAAAGAAAGCTTGATAATAATGGTAAGTGGAGAGGTGGCCATGGCT
GTTGGTACAGCCCTCCTGGTTTGTGTTAAGTGATGTTCCCTTGGTGCTGATGGAGTTCCTTACCCATTCTTCAGGCTGGGGAATCTCTCTTGAA
AGAAATAGGAGCTGGCCAGAAAGGCAGAATGTTTCGCTATAATTGAGATATAAATTTTAGCTTTGGGTTTAGAAAGAATGGCATAAATCCCAAG
ATTTTCCTTCTGTATGTATGCTGGGGCATTACCAAAATGATACCCTTGCTTCATTTCTCATCTGTCACTCACACCTCCACACTGGTAAATGCTG
GCAGAAATTGTTTGAGCTTGTCAGAAAAGTTAAGGTCTGAAACTGTTGCATTCAGAGGTACCACGCAATCTCTTCAGACCCACATGTGTCCCAA
CCAGGATAGGAGGTTTGCTGATCAGAGATGCCCCCATAATTGGGCTGTTAGTAATAATCTCACTTCAGGAAAGAAAGCGAGAACAGGGTAGGCA
TGTTCAGAAAGCTATCTGGTACATATTCTTTGAGGTAAAGCATCTTTAAAAGGTACTGCTTCCCCAACACTATGCATGAAGACTTGAAGAAGAG
TAAAGTCCTTTTGAAGAGTATGGGTATCACCCCACAACAAGCAGGGACCCTACTTACTGGAACAATAAGCTGGCCACCATTTTGTTTCAGGAAC
AAACCAACTAGACCACTATAATAGATACGGATGCCGTTGGCAATGTATCTATAGGTAGATATGTAATAAAATAAATGAGATAGGCTCGAGCAGC
TTCAACGAGCGGTTGTCATGTTTAACAGCCATTAGTAGATGTCCAGTCTCTCAGTAAGATCTATTCACACTCTGAAAAATGTGCTGCCAAGTAC
AGGGAGAGGTAGCACATGGGCCATGTGGGATAGGGTTGCTGTAGGAAACCTTGCCTCTGTGCTGTACACAGGCAGATTCCAAGCGCATGCCCAG
GGACCTGGCACAGGCTGAAAGAGTGGTCACTGTATGGAGTTGCCAGATGCCATGCTTGCTTACCTGGCTAGTTAGCTTAAGGGCTTCATTTTCT
GATCTTTGGCAGAAAATCTCTCACAGGACTATGGATCCAAGTTTTCAGATTCTTTAGGCTGTCACTGACTTTTTCTTTGTTGCTATCCAAAGAA
CAAATACTTGTTAAATGCCATTCTGAGGCCAAGTAGCTGCCATCTACTCTAATTTTGTTGGGGACATACTTGCCACGCATATAATGAGGAAAAT
GGGGGTGAATGAGATGAGCTTGAGTTTTTCTGAGGGGGCAGGATATTTTAAAAAAAAAAACCTGCAATGCATACAACAAGCAAGGAGAGAAATC
AGTTTTGAGGAGACATATGTAGGTCTTTAGATGACATATTATGTGTCTTCAAATATGGTGGAAACTGGCTTGCCGGCTTTGGCTTGGGAGTGAC
TGAAGGCCATTGAAGGAAGGCTTGAGTCAGGGATAGTCTTCAACAGGTATGAACAGGCACCCAGGATATGTAATGTTGAGAGTGACTTGGGTGG
CTTTCTCATCATCAAGTGATCCCTAGTCTAAAGCATTTGTTCTCTCACAGCCTTTTCCAAGAAAGCTGAGAGCATGACTCATCCTTATTAAAAC
CACTTGTGGCTAGGCTGAGGCAGGAGGATTGTGATTTCAAAGCAGCTGGGGCTACACAGCAAGACCCTGTCTTGAAAAGGAGTAGAGAAACTGT
CACACTGAAATTCCTGATTCTTACATGTTAACATCTTGAATGGATAAAGGTATGACTCAGTGGTAGAGTACCCATCTAGCTTGTGCAAGGCCCT
GGGTTCAATCCCCACTCCTGTGAAAACAATGAAGAGTGGGTATGTTATGGCATTGATTGAAAATAAGCAAGGGACATTTGAGGACCATCCAGGT
ACAATTTGAGTCAAGACTCTCAAAGCAAGAAGGCCTGTGTGTTTCAGTTTATAAAGGTATTCAGAGAGCCATAGGGTTGGAAGAATCAATATGA
AATAAAAAAGCCAATTTGAAAAGGCAAAATGATTCCCCAATGAGAAATACTTCATCAGCTACAGGGAGCATGTCATGTAGAGCATGGCTGCCCT
GGGGTGCACTTGGAGATGGTTCCTGACTAACTGTGCTGTCGCCTCAGTCTCAGAACTCAGGGCTGACACCCTGGGATACTTGGCCAGGTGGAGA
CATGATGTATTCTGTACTTTGTCTCCTTGAGCTTCAAGTCTGATCCCTGCTACCGGTAGTGCAAAGACCTACGAACTTTCCTCATCACCACTGG
TCCAGACCCTCTGGCTGCTGCCGTAGCTTTGCAAGCTATGGTTAGCACCCTCCTAGAAATCTAGTGTGCCAAAGCAAAAGCCGCTCCCACTCTC
TACCCTGCTCTTTAAAGAGTGTGTGCTGTGAGGGGCCCTGCAGCTGCACTACAGACCCTCAGCAACTGAATGCTGCTCAACGTTGCCGAGAGGT
GCTAACCTTGCCCACAGAGAGCTCTGCTCACTCCTGACCGTTAGAAATGTGTCACCGAAGTGTGAAAATACTTATGCTGTTAGACTTCCCATCA
TTTCTTTTCTTTCAATCCCTCTCATTTGCATGGGTTACTCATAAATGTAGATCTTGGGTATGATTTGTACAACTGCCCTGCTGTCGGTCTGTAG
GAAAGCCTTGCTGGATGGGCTTGTCTCTGGCCTTCATCTACAAGTGCTGGGTGCAAGTCAGTCACAGGTGGTGCCACAGGCAGCTGGCCTGGAG
CCGGGGGTGCAAAACACAGAGTGATAGTCGTTTGACACAATTTCAGCTCTCAAACCTGCTTTGTAAATGAAAACACTGCTTTTGGACTAAGTCA
TTCTCTCTGATTCCCAATTGTTCAATTAATCTATGTGTGTTAACACCTCTCTATAACATCATAGATAGTGCCTAGTAATGAACTCTGCGAGTTC
AGGCTGAGGAACCCTGAATGATCCGCCACTCTGCAGCTGTTCAGGAGTTGTACCCTTTTTGTGATGTGGTGAACTCTTTGAGGACTTCAGCACT
GTATTCTTAAGATACTGAAGGAGTGGGTTGAACTGTAGTAACAACAAGGATTTTGCTTTTTAAAAGGATGTCCCTGCATACAGCTTAGCAACCT
TGATTGAACTCAGCCTTTCATGGTTACACCTTAAGGTTGAAACCCATTTCATTTCATCATTTCCCAGAAGGGGCACTCAGTTCACCCATTTCCT
GTTTTTAATCTCAGGTTGTTCTGTGCTTGTTCCACATCTACTCTCCCAGTACCATGGCTATGGAAGCCAGCGTCACACTCTTGCCCCACCTGGC
ATCTATGCATTTTGCACATAGCTGCCATCCGTTCTAAGCTATCTGTGCACAGATCTGTGGACAGGGCTGGCTGTTCCCGTGGTGTCTGGTGCCA
TGGCCACTCATGGAGCTCGTCACCCCAGCACTGTTGTGCCATGTCTGACACACAAGCTCTCGTCTCCCTATGTACCACAGAGTCTCTTTAGTAT
GTCACAGAGGTGGACATGGTCGTTAAGACCAAAGCTTCTAAACTTGCTCCATCCTATTTCCAGTGGACTTGAACCACTGTGTGTAAAGAATTTT
CATTGACTGTGCTTGATACCTCCTGCCCCGACAGTCTTTAAAGATGTGTGTGTGGGGGTGGGAGGTGGGGGTGTCTGTGTGTGTGTGTGTGTGT
GTGTGTGTGTGTGTGTACTTGCACACCTTGTTGTTCCTAATGATGTTGAGTGAGACCAGCACATGACTGGCATCTGGTATACAACTGAGCCATG
CCCACACATCTACATTCATGTACCCACCCCTTCAGCGTGCCCTTCCCTGGCCTCCCATGTAACCAGAGTATTGACCTGACCCTTTCAATTCTGC
TCAGGTTTCAGATGGTTAGGACCCTAGGGACAGGAGCCATGCTTACGTTTGTTAACCCACAGGTCCTGCGGTCCACCAAAAATAAGTTGCCTTG
CCTGAGTAATCCTCGCTCTAAGCTGTCTTCTCTGTTCTCATACTGAGTCCTGTCTCTTCCTCTGTTAGGCTGCTGGAGATTAGATAAGAAACCG
TCTTTCCCCCCACCCCCACTACAGTCTGTAAAGCAGGGTCCTCACCCTCAGAGTGGGTGTCTCAGGGATTTGCCAGGAAATGCATGAGTTTCAC
TTCAGAACTTTGCTTATCTGGGTTTTAGCTGTTGTTTCTTTCCCATCCTGGGACCCTTTTCACTGCGACGTGCAGTTGGATGTGAATTACTGAG
TGATGGGGTCGGGCCAGCTACAGAGCTACTGCTCTTCTTAAGAGTGTACAAGTTCTTAGCATCGTTAGGCCACAAGACTGTCACTTTCAGCCTC
TGGTGTCTTTGGATTCTTCCTAGTTAAGCTTCTGTTGTGTTTCTGTCGCTTAAAAGTGACAGAAGCCTCCAAGTTGATGTCCAAATATGTTTCT
GTATTTGTAAAGGATGTGTTTTACAGGGGCTGGAGAGATGGCTCAGTAGTTAGGACACTGACTGCTTTTCCAGAGGTCCTGAGTTCAATTCCCA
GCAACCACATGGTAGCTCACAACCATCTGTAATGGGATCTGATGCCCTCTTCTGGTGTGAGTCTGAAGAGAGCAATAGTGTTCTCATATAACAT
AAAATAAATAAATCTTTTAAAAAGAGAGAGAGAGAGGAAAGGGGAAAAGAAGGATGTGTTTTACTTGGAACCCTCCTTGGCATCCTTTGGCAGC
ATCAGTAATGGAGACATGAATGTTTCATCGCTGCAGAGTGCACCTTTGCAGATTTCGTGCAGTGCCCGTTTCCGTCAACCGTTTTAGGTTGTTT
AAGCTTTGTTGTTGGAGAATGAGATGGTGTGGTGCTCACTGTGCTGTACAACCTCGCCTCGCTTTCACGGTCCGATTGCTTCATAAGGTTGAGA
GACACAAAGACCATGAGAGTCCTGGAGGCTGCAGCAGGACTGGCACATTGGGAGAGTGTGTGTTCCTGCACCAGCAGGTTTCCATTCACGATTT
CCCAGGAGCTTGGACTGGCAGGCCCTAATCTTCCTGACATCAGCCCTTCCCCCATCACTGGCAATGACAAGCTGTTTCAGCTTTGCCACCTCTC
CCAGGCCACTGTGGATGTCACCGCATAGCATGGCTGACTATCTAGTGCAGTCTCTGGCTGACACAGGTTAAGTGGCCATGGGTCACAAGTGATG
CTATCCTGGTCATATTGGTTAAGCTATCTATGTGACATGGCACACGTAGGACAGCCATATGTCACGTGACTACATTCAGTGATACAAGTGCGGG
CATCTACATGACCGTTAGGGTATTCTGGTAACAAATGGCACAGATAAGCACAGGCAACCAGAACCAGCACCTCCACCTAAGCTTGAGTGATCAG
TGGGGTCCCAGAGGCTCTGCTGGCAGAGGGATGGCCAAGGGCATGATTGATAAGGCTTTGCTCCAATGTTAGCTGTGTTATATCAACTGTGTTT
CTTTTATTATTTTTGTGTTTATTTCCATAAATACCTAAATTGTTAGTGAGATACTAACATTTCATTGATTACAGAGTTTCAATGACCCTCTTAG
TCGTTCTCCCCCAAGACATTCTGTGTTCAAAAATATGATGGTTTATATATGTGACACATAGAAAATACCATCCAGAGCGTCTGGCCAAGAATGT
GCCCATCAGAAGCCTTGAAGACAGCATGACAGGTGTAGGGCTGGCCAGGGTTTCTAGGGAAGGTGGACACAAGAGTCTTGAGCCCTCTTCTACA
TAGTCCTGCCCACCAGCCTCCACTAGGGGAATCTTCCCCAGCTTCCATTTTCCAGGGAAAGCCTGAGCCATCTGAAATTCCTTTCACAAAGAAA
CACACCTGGCCCATACAGGTGGACAGGTACCTTGTGCTGTGTATCATCTACGACCCAGTGTACGTAATATCTGAGTAGAAAGGTCTTGTGTAAA
GAATGCTCAGAGCACACACGAGCACAGAAGAGCCTTCACCCACTCAGTGCATGGCCTTCCCAGGGCTGGACATAAAAACACACAGATGTGTGTA
CACCACACAGTACTCTAAGCCACCAGTCTCACGTCAAAGTCCCTCCAAAGTTTCAGATGCTAGATGGCAAGACAGTAAACAAATAGGAACAGCT
CTTCAGAGCTACTAGCATAGAAAGTCTCCATGGACATCAGTTTGTTGTGAGAGCAACAGTGGCTCTGAGGTGGCTGATAATTCTCTGGAGCAGT
GAGACGGTTGGGACAGAGGCGTGTGACCTTTAATCCCTGCCTTGGTGAGGGTGCCTTCCATTTACACCAGGCCGCTCTCTCGCCTCCTCAGTGG
GGCCTGCGGGCTCTGCCACTAGCCTGTGACATGTTAGATAAGGACTTTTTCTTCTTGGTGTCCACACTCTACTAGTGGGCTGCCTTTGACTCAA
CCCTGTGCTTGCTGATTATCTCCACAAACAGAGTGCCACAGACTAACACGTTAGTTTGGAATTTTCCTCATTCTTCTAGGATATTCTCTTTCAT
CTGTTCTCTTCATTCAAAAAAACTGAGTTGTAGAGGTTTCTAAATGTGCCCTTTTTAAGTATTAATATATATAAATATATTCTCTGTTTCAGTA
GTCTACTTTTGTTGCTTGTTTGCTTGATTTTATCTCAGTCCTAACACTAGATTTTTATTAAAATGTAAGGAAATCTTATTGTGGTTTTTATTCT
CGCTGTTTAAATACAAGAGAAAAAGAACTGAGTGGCCTACCAGAAGTATCAAGAGTTCCTAGATTTTAGGATCAAATAGCAGATAGAGTCAAAT
ATGCAGGGCACAGTTGAGCAGCCCCTCTCATCCCACAAGACTAAGGTAGCCGTCATACTCTTCTGTGGAGAGTAGGAATACAGAGCTCAAACTC
TCATTTTATGTTCAAGAAAAGCTGTGTAAATACATGTATACATGCCTGTGTGTGAGAAGACACATCTGTGCATGTGTCTCTTGTATTTTTGCAT
CTGTATAGGTCTAATGGCATGTTTGCTGTTGTTCCATCTTGTGTGTGCATGCCTATTCTGTGACAGTATCTCTGCATGGGCCTATATTTGTGTG
TCTGTGTGCATGTGTGTATTGTGTGTGACCCAAGCTCTCTGCTAAACCCTCTGCATCATTCCCTAATCTTACTTCCCTTTCTGTCAGTTCTCCA
TAACTCTTTGATTTCAGTTGGGCTGGTGTCTTGAGCAGCTTCCTCTTTTCCTGCATTCCTATATTTCAGTGTTCACCCTCGTTCCTCTTCACAA
GCTCTTACCATACAGCCAAGAGGATGTGGAAACCCAGTTACAATATGTCACACAACACAGTGTCACAATCACCAAGCATGTCCCCACCTCCTGG
CGATAGTGATCAGGATGGGATAGGAGAGGCAGGGAGGGACATGGACAGAATAGAGAAGTTTGGCTCTGGGAAGCCCAGGACTTCCTGGTGCGTA
GCTGCTATGCCAGATGCATGGTGAGCAAAGTAGACCATTCCATCATTTTCTACAATGGCAGTTGGGGTTCACACATCTGCCTTGGGGCTAGGGC
CTCCCCGTCATTGCCTCATAATCCTGCTTTTCCTCCAAAATGCCTGTTCTGTTTGGCTCCCAACTTCCCTATTTCTGCCTGTTGGGTGTTATGG
TCTCCTCCCCTCCTTGGAAGTTTCTCCATCCCAAAGCCAGTTCCTACCACGTGACCAGCTGAACAAGCACCTCAGACAGTTCCTCTTGTCTGGG
AACTCCAGCTTCCATAGGGCCATAGGATGGCTCCCAGCCTATTATCTCCCCCAGCCCTGTGGATTTCAGAACACTGTCTCCATAACTCTGAGTA
GGCAGAGGATCACCTACATGCCCAATACAGGTCTCTTCCCACATACCTGACTTCTAATGAGCCCCCCTAGGAACCCAGGGCCCATGGTCCATTT
CTGCTGGTACTCCATGATGGCTCTTGGGCAGTGGCTATGTGTGTGCTGTATCTTATAGTACTGCCAAGCTAAATGCTCCTGAGCCAGAGACATC
TTTCTCCTACAATGTTCTCCAGTGACACCCTGGGGGTTAGAAGCATATTTCTAGAATGGCTGCATTCCTCCAATGATGTGTTTGGCTTGTTGGA
AAGGCCTTGTTTCAAAATCTATTAGAAGCAGAAAGTTTATTTAAAACAAAACATGTCAGGATTTAGGGAATTATCTGGGAGGAACTGGCTATGA
GGTCCCATAAACGTGGGGTGCATCCATAGCTTGCTGAGTCTCACTAGTAACCCTGGGCAGCTCTGTTGTCTGTGAGTCTTACTTCTGTCTCTGG
TAAGATTGGGGAGGGCAGTATTTATAAGGGTTGCTCCAGGAATTAAACAAGTAAATGCATGGTAGATGTTCAACACAGCTCCCTCACCTAACGA
AAGCTCGCAAGCTACTATGGGGACTGTGGGGGAACCAGTAGCCTTGACCTAGATGCTAAGTGCTTAATACGTCTGATTTTAACTGGGAAGAGCA
CATAGCTTAAACTACTCATTTCTATACATTTCCCAATAAAGTTCTATTGAATCATACAATGAAAAAAAACAGGAAAGAAGAAAGAAATTCTCTC
ATTTCAAAGAAATGCTAATAAGGACAGGACTTTTTGCAATGGCCTGGTGGTAAGAAATGGCTGGTCCCTACTAGGAAAAACATGGTATTTGAAG
GTTCTTCCCAGGAGATGTGACATTTTGATGAAAGTGCCAGTAAGCTAGGGGTTTCTGAGTAATGAGCTGAGCTCAGAGAAAGGTAATGGCACCT
GCGCAAAGGGTGCCAGCATTTCCTGAGTGCTGGGTGTCTTATCTGCCCACTCAGACCACATGGAGCCCAGAGACAGCTATTTTTCTGGATTTCC
TTGGGAGGAAGCTGAAACTTGGTGATGTCATGTGACTCTTCAGTCCCACAGAAATGATAATGTAGGGGTAGGAGGGCCTGAATAGAACTTGCTT
GCCTCTGTGGGCCCATTTTCCATGGCAGTTTCCAAAGCTTGGAGGCCAGATGTTAGGACCACAGTCATGACTAACACCTGACAGCTTTGTAATG
ATGGCCAAACAATATAGAATGTGGTCAGCATTTGTAATGCTCAGTTCCTGTAATGGGAGTCTAGAACAGAAAGGAAGAAGAAGAATAAAAGGGA
GAATGACTCTTGAAGACCTCTCTACAAAAAGAAACATCCAATGCAAAGGCTTTGTTGTGCTGAAGTTAGCAGTAAGTTCCCCCTAGACAGTGAA
GGGCTTGGACTCCCAGCATGTCCTGCCTTCTCACCATGTGAATTAGCAACCCCCAAATCTAATTCTGCCACCAGCTGCCATTCCTTGGCCAGAC
CCTCTTAACAGTGGTTCTCAACCTTCCTAATGCTACAACCGTTTAATACAGTTCCTCGTGTTGTGGTGACCCCACCCATAAAATTATTTTTTAT
TGCCACTGCATAACTATAATTGTGCTACTGTTATGAATCACAATGTAAATATCTATCTTTTCAATGGTCTTAGGTGGCCCACATTAAAAGGGTT
GCTGGACACCTCCACAAAGGGGTAACAAAACACAGGTTGAGAACCACTGCTCTATAGAAATGTGTTTCTGTTCAGATATAGAGGAAGCTCTTGC
AGGCCACATCTAACATCAGGTTCCTGCCCCCAGAGCCAGAATTCTGGAGAATGTGTCTTAGCATGGGGATAAGCTGATCTCATATTCAGAAGAG
TAGCAATGGGTCCCTATCTGTACTGTTCTCATCCTCCATGAAAATGTCCCTGTAAATATGATCTATGTGTGCTACACACCTGTACTGATGCACT
CTTGTCATGACTCTGTATACACACAACCTTGTGCTCTGGTACACCAGGCCAGCCAAGTACATCCATCCCCATGAGCACACACATATCCCAGCCA
CTGACCCAAGCTTGTTCTCCTGTACACATAGCCAGCTCTTGCAGATATCCTCTCTGCTCCTCCCCATCTGAGCTGGCACCTGAGAAACAGGTGT
TCCTGGGAGGAGCACATGGAGAAATAACCAAAGCAGAGCTGGGGTTTCGTTGCCTCCTGTGAGTGAAGTTGTCAAAAGCCTACTGACCACTCCA
ATCCTGATTTCTCAGCCTGAGTGTGCGTAAGTGCCTCTCCACAGACAAAGGCACACACTGCCAGCCAACACCTGGACCTGTGGCTGCAAAAGCT
GCAGGAAAACCGACATCCTTCCCATCAGTCCCCCTTCTTTGCTGAGTCACTCTTTGCTTGGAATTCTGACAGGTGCCCGTTGGGCTTCTGGAAG
GGAGGGACAGCAGAGGATCCACCCTCTGTGTGTCCCACGAGAGATTACATTATCTTCAGCCACCCTGAAGCTCGGCCTAGGCTTCTGGAATCGG
TGGGGCTGACACAAAGCCAGTTCTGGGGACAGGGAATGTACGGATAGCCACAAGGAGCTAAGCCATCTGATGTTCTCTTCACCTGAGCACCCTA
GGACTTAGAGTCCTCAGCTCTGCTTTGCATGTGTTCCACTCATTTCAAGGACCAGCATACCCTACACCTCACCCATGTGAGAGCTGCACGCTTC
TGAACTATATTTAAGAAGCCAGGTCAAGTACAATCCATGTAGTGTTTTCAGCCCTACATTTTGCCAATTGCTGGTTGGCATTAGCAGGCAGTTC
AACTTGTTGCTCTTGGAAGTATGTTGCTGGGAAGGTCAGTCTACCAGCCATGTGTAACCTCTTCACTCACCTCACACCTAGCAGTGGATGGAAG
CTCTCATTAGCTGGCTCATACCAATTTACTCTGATAACGACGAAGCTTGTCCTGGGACCCTTCCCCGCTTCCTCCCAAGCCTCTGTACTTCTCT
GGTGAACACGGCCACCTCTTCACTTTGTGGTAGGCGCCTCTGACTATGTCTCACCAGCACTTCTAGGGCCTGAGTTTTGGAAGATAAACACAGA
AGCCTCCTGGTGTCCCTCCTGACATCTGCAGTTAGTGCTCTGGTGCATGGCCTAATTATCCAGCAAGATTCCAGTGTATACTCTATCCTCAGTA
TACTGGCTCATGTGATCTAGTAAAGATGTCTCTCACTGCACAGAATTTCAAATGAAATGGGAAATAAGTATCTCAACTTTTAACTCATACCCAA
GACCTCAAATCATGTCACCATACTATGAGACACAAAACAAGCACCTTTGTAATAATGTTCCCCTCCTCTGCCCACACCTTGTTCACCTTACTCA
TCAGAATACTCCCATTCTCTGTTTTTAAAGCTTTCATTATTGTTTTAATTTTCTCATGCTTATGTGGTTCTTCTTGAATCCCAGGTCTTACAGG
TGATGTTTAGGAGCTTTCTCATGGCAATTTTGCATACTTTGTACCACACACATGAACAAGTAATGCTTTTGATGAAAATGTAGCACTTCTTTTA
AAAAAAAATCAAAAAAGCAAGAAACTTAAAACATTCATAAAATATCTCTAAGCTTTTTCCCCCAGACAACCAGATTTAACCTCCCAGCTGTGGG
TCTGTAAGACCCAGAGCCTGACCTAGTTGACATGAACTGTGTGTAAGAGGGCCAGGAGAGAACAGCTCAGTGTCAGAACCTGGGGGAATTACAA
ATGGCACCATAGTGATGACTTGTGGCTTCTCCTGTAAATCTGAATAGAAACATTATTGTTGCTCTCTCCTCCTCCTCTCTAAACGAAATTCTGA
ACCCATCTCTGTATTTCTAAGTCATACATTGCTTTTTCTAACCTGATTTGTTCTCTGTGACAATTTAGTACTGTGTTTGTAAAATAGAGACAAA
GCCAGGACAGGGTTGGCGGTGTGATTCAGTGGGAGAGCACATGCCTCATGTCTTCACAGCCCTGGGTCCTGCCCTCAGCACTGAAGGTAAAACT
CAGGTGTTCTACAACAACCAGAAAAAGGGGGACATTGTGTCCTCTGCATTCTCTGACAGGTGGAACTGGCAGCAAGACAAACTGATGCTGGGAA
CGACCCCAACGTCCATGTCCCCCAACAGCCTAAAGCACAGGGAAGATCACCTCAAACATACTACACATGAGATGAGTCCCCAACAAAATCCTGA
AACAGAAGAACACTGTTAGGGATTTCAGGGCTGGAACATACAAACATCCTCTACCCTGGCTCCATTGTCCTGGGTGAGCTGCCAAGCCCTCTTC
CTGCTGCTCTTCCATTTTGAGGAAGGTGACAAACGGTAGCTGGGTGTAGCCAATGTCATAGGCCCATAGGATTCTTTGTGGACAAGAAGATAAA
GCGTTATCTTCCCAGCCTGTGGCTTTGTGCTGAGCACAAAGCTAACTACTTGTCCTCTCAGCCCCGTGGAGTATTATCATGATTCCGCACACAA
ACCTGACAGAACAGATGGGATTATTCCCACTTGGTGGATAAGGAAAGTGAGCTCAGATAGTCAGCGCATGATACAGTTTCCACCAAGGAGGAAA
AGCCCAGCATCAGATGCCTCCCCCGACCTGGACATGGTCAGCTGCTTCTTCCTTCCATGAGATCATAGCCTCTAGACTTACAAATGTCTAAGTT
CAAGAACATCCAGAACCAATGGAAGCCAAGGGATTCATTTGATTGGAGAGCCAGTAGGCATAGACTGTAGTGATGTATGACACTGGATGAAATT
AATGTCAAAAATTACTTTCCCCACTGTTGACTTTGCTCTAAATATGCTTGCGTCCTAAAACTGCATGTGAACACCAACATGTTCTGCCATGTAC
TCTGACACTTCTCAATTACTGGACATGGATTTCTGAAACTATCCAACTATTGTAGTTATTAAGTAGAGAGCTTTCTAGCTGAACTGTGTCACAG
ATAACCTGTACCTCCTGTGCATGCTACCAACTGACACTGAGTCCTAGTCAGCCATGGCCTCTTTCTGAGCATGGCTATCGTCAGGTCACATGAG
CATTCAGAGCTGGGCATTTATCTGGAATACGCTTATGTGAAGACCAAAGGCCAGAGGGGGGAGGGTCTTCTGAAGAAGCGTCTCCAGGCTATGA
TCATCGGGCAGGCCCTGCAACTCCATAATACTGTGAGGACCAAACACAAAGAGTCCAGAGAGTAGGCAATCTGGTTCTAGGGGAGAGATGATCT
GGGTGTCTCCATCTCAGACCCAGAAAGTCACTTAAGCCACGAAGACACATGCAAATGGTGGCTGTGGGGTGGAGGGTGGGGCATTGCTCTGGAA
CAAGAATGTGCATGGTTAAATGGAAACCATAACATTTGCAAACATGGGTAATTTGTGAGCTTTGGTTATTGTTTTAGCATTTTCCTAAGGTCCA
TCCCTTTATTTATTGAATAGTGTGATTTGACAAGAGATCTATAATAGCAGTTGCCAGAAGTAGAGGAATTTTGACACAAGATTTTCAGTTCTCT
CACACAGCACTGTAGACTGCTGGGCCATATGGAAGTCAGATGTGATCATAGAGACTCAAGGAAGGTGACAGCCTCAGTCTGATAGACACGTGTG
CTGCGATGGGAGCCCAAAGGCCACAGCCCACAGTGCACAAAAAAACCTAGGCAGGTGGACAATGGTGAGCAGGAAGCACAGTCAGGAGGTAACA
CAGACCCAGCCTGGGCTTCGCAGTGGCAGGGGTATGATTGGAGCACATCAGGGAAACTGAGGCTCCTTCTCAAAGAAACTGGGATGATCATTGG
ACTTTGTGGGTGGCCATGGAGGAGCTCTGGTGGATTCATTGGCTAGAACCTTGTTTACTGACACACATGGAGAAATAGGAAAAAAGCTAGGCCT
TGGGGAGAGGGTGGGTTTGGAGATGGGCAGCTGTCTGGTGCCTGAAGGGCACAGGAGTGGCAGGCAGGAGGCAGCCCGGGTGCATCCTGTTCTC
TGGGCCTTGGCACAGTTCTGCTGCCTGCTCTTTCTATACTTGAATGCCTGGACGCATGCAGAGCATCGTAGCCTACACAGAAGGACCTCTGATG
TCCCAACAGGAGATGGTTGAAAGAGTTGGGACAGCCTCACAGCTCTTCTGACTCTGCATCTGGGTGAGTGGTGGAAGTCACTTGAGTAGGTGTA
AGACTGCACACCAATGCACCTCTCCACTGTGTCCACAGGTGGGACGTGGGGCTTCTTTGTATCTTAGCTGTCTCTTTGCAAGGCTGAAACCACA
ACGTCTTCTGTCAGTGATGTCATCTAGCTAGTGTCCTCTTCCCGTGAGAAAACCCTGATCAGCCCAACCAACAGGGAGGTTTAAGAAGAAATGT
ATCTGTCTATAAAATATACTGGTTCTCAAAGAGGAAGTGCTTTTGGCTTCACCCTACACTCCTGTAGAATATACCTATANNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNGAGGCCGGCAGATGGATCTCTGTGCATCCAAGGCCTGCCAAGTTTACATAGCAAGTTCTAAG
ACAGCCAGAGCTTCATAGTGTGATCCTGTCTCAAAAAATTTTGTTTTTTAATCTGTGCTATTTCATAAAACAGTGGCTTAAAGCTGCAGGTAGA
CTGTCTGAGAAAAGAGGACAAAGCCAAAAATCGCACCGTGCTCCCTAGGTCCCATTGTGGGGTGAGCTGCTTCTGATGGGATACTGTATAGACA
GGGTATCACAGGGCAATGAGCTTGCACCTGAAGATGGCTGCAAAAGAGCAAGCAGCCAGGAACTGTGTCCTGATTCAGAGCTTCTATAAGCAAT
CTGGGAACATTATCCAAGGTACTCTCAGTGACCCCTGAGCTTCCCGCCTCTGTGTGTACATGAAGAACACCAGGCTTAGCTGTGAGGACCACAG
AGCAGGCACAACTAGGCAGTTGTATTTGCAACACCCTGCAAGTTATTAGCTGGCCACGTGTTTGTCATTCGGGAAACTCTGTTGTAAATGCAGT
CACCCAGTGTGACCATATTCAGGCAAGTAAACACATATGCATGGGGGTGCACACTTAAGTGTGTGAATGCAGTGGCAAAACTTACACATTTCCC
AGTGCCTTACAACCACCCAAAAGAAGGCTGCTTGCAAAACTCTGTCTTTTGAATTATGAGGCTCATGTAACTAGCTTTGACTTTAATTTTCGTA
TTTCTGTGCTAAACACCAAAAACTTTACTTTTCACATTTACATCCTAAACACTATGCTTAATGCAAAGTATAGACCTTCCTGTCAGCCCAAGAT
CCCTTACTGCCAAAACAGTGATGGTGTCTACCTCAAAGACCCACAGTGCAGCTTAGAGGCTCCCTGTGGAAAGCAGTTTGTGTGCACCTGACAG
ACCCACTGAGTTGCTGCTGTGAGAGACTGGATTTTAGCATAGATCGGGGGTTTGCTGTTTGCCGTAATTCCTACCCACTTAGAACACCATAGAA
TGTTTTTTCTAAGTCCAGCCATTGTATCTGTATCAATTCCACTTTACTATGCCAGACAGAACTTAGCAACTGGTGCTATATTTTGCTTTTAAGC
TTTTGGTAGATTTTTGGTTAAGGATGAAAATTAGCAGATTGACTTGAGTTATGGTAGACAAATGATTAATTCTTTTGAAAGGGACTCAACATTT
CATGACCCAGCATCATGGTCTTTACATTAAATAGTAAAAATGTAAACTCCAGGCATGGCAATGAACACCTGTAATCCCCACAGTAGCAGAGGGA
GCAACAGGCGGCCCACCCCTGAGCTTTCTGGCTAGCCAGTGCAGTTGGATTGGCAATGTACAAATTCAGTGAGAAGCCCTATCTCAAAAGATGA
TGATGGGGGCTGGAGAGATGGATCAGCGGTTGAGATCACTGACTGCTCTTCCAGGGGTCCTGAGTTCAACTCCCAACACCATAATGGTGGCTCA
CAGCCATCTCTAGTGGGATCTGATGTCTTCTTCTGGTGTGCCTAAAGACAGCTACAGTTTACTCATATACATTGGATAGATAGATAGATAGATA
GATAGATAGATAGATAGATAGATACATAGATATTTTTTAAAAAAAAAGATTTGATGGTGATGATGGTGATGGTGATAATGTCTAGAGCAATTAA
AGAAACATCAGACGTTGATGTCTCCACATATGTATCCACACAACTGCACATGTACTATCCACATACATAAAGTTTTTCACGGGGAAAAAGGGAA
GGTTCTGTGCCCAAACAAATTTGAGAAACAGTAACTTAAGCAAAGTTTGATAACTATGTCTTTTTCCTGAAGTCCATCAGCCTTTTTTTCCTGC
TGAGTCAGTACTTGATCCCAGAAGTTTAATTTAGCCAGCTACTTTGTGTATACAATTCTGGCTGACATTTTGGGAAAGAAGTCATGTGAGCGTG
CACCCGGTGTGCATGCATGTACATATACACACACAGACACGTGTACAGAGTATAACAGGTCTTAGGAATTGGAACTCACTAGGAACGGCCTGCG
AAAGACTACAAGCACGGGGCTTGGTGCCATTTTCAAAGGCAACTACTAAGCAATACGGAAATCTGGTAGCTCCTTTTGGAGCAGCAATTCCCTA
TCCAACAGGCAGATGGGAAATGTTTTAATTTTCTCTGGATACATAAAAGAAAAGACAAAAAAAAAAAAGCAAATGAAGAACGTTTGAGGGGCAG
ACTACCCATGAGCAAGCACACTGATTGTGCAGACGTGTGCTTTCCCGTGTGAGACTGTGATGTGGCCAAGAGCACACTCAGCCAGTACTACTGA
ATGGTTGCTATTGGTTCTACGATGTTCTAGGCTGTTGAGGCCGTCGCAGCTTCCTCACAGTTCAGAGGTCCTGCTCTGAGGAGTGACTTTTCTG
TAGCTTGTCTGAAGACAACAATAACAAATTAGCAGCTAACTGGTGGTTTTACATAATAACATTGCCTTTTGAAAGCAGCACTGTCTTCTGCACT
CTTGTGTCCGTGATGATTTGACAACCTGGCTTTCTTTTTGCCTTCTCTATTGCAGTTGCATATGGGGCTCATGGCTTTAGGGATTTTCATGCAA
TAATTCCCAAATCTTTCTCTCGTAAGTATATGCTTTGCTTCTAGAAAACAGATGTTCATTTCATGGTCAGTTCCATCACACAGACACCACCACT
TGCATGTTCTCTCATCAACACCTTCCTTTATAACTTGAAAGTGACATGGACTAAATGTTTGCCATTCTATCATTAAACACCCAGCATCCAGTGT
GTCATTGATACAATAAACTGCAAAGACAGACCGTGAGGGTGTTGTTTGGTTTTTGTTTTGCTTCTGAGGCAGGGTTTCCCTATTATTCCTGGCT
GGCCTAGACTGCTCTGGGATGCCCAGACTCATCTTAAATTTGCAATGATATTCCTGTCTCTGCCTCTGCCCCTGCCCCTACCCCCTCCCTCTGT
CCCTGCCCCCTCCCCTCCCTCTGCCTCTGCCTCACCAATAAGGATCAGATAGTGAGAGGATTATAGGGGACTTTTGGGATACCATTTGAAATGT
AAATGAAGAAAATACCTAATAAAAAATTGGAAAAAAGAAAGAGAGAGAGAAAGAGAGAGAGAGAGAGAAAGAAAGAAAGAAAGAAAGAAAGAAA
GAAAGAAAGAAAGAAAGAAAGAAAGAAAGAAAGAGAGAAAGAAAGAAAGAAAGGTGAAGAAAGAACTAATGTAGCCACAGTGGGTGTCTCACTG
GGAGGCTGCTCAGCAGGGTCCTGATTAAAACTGTGATCAACAACTTTGTGGTCCATGCTTCCTGTCAGCATCAAACAAACCAACATCTTTTCAG
ATAACTTGTGCAATTTTGTGTGACAGGAAAAAAAAAAAAGGTGCACCCTTTTTCCCTTGCTCTGGAGTGCTAACCTCAGAACAGAGAAGAAAAA
CTCATGGGTTTCCAGCCCATTCTTCTGTGCTGGCTCGCCACGTAACCATGCACCATGTTAGCGTGCATGCAGCCTACCAGCTCTCCTCGGAGCT
GCTGTGCCGAAAACTATATGGTAATTTCATCTCTTAATACATTGAAAGTTGAAAATGATAACTATATTTGAGCAACCATAAGGAGTATTTTCAA
GAATAGAGAAGGCTTTTTTTTAATGCAAATAAAATCAATGCAAAAGAGTTACAGGTTGTTAGACCCCTTAGGTCTTAAGCCTTCAGACCTTGTA
TCAATAAAATGTTTAATCTTATGAAGCAGCAGCAGAAAGCAGAGTGTATTGGCATCACTTTGAGAAGAGCTTTGTCTGTGTGTCTTGCTTCAGA
TCAAACCAAAGCACACTCTACCACCAAATTACACCCTAAGACCAGCTTGCTGACATCTTACTACTCTAATCTTAATATTGAGATGTCAGCCGCG
TTAGGTTTATGATGTCCTGTTATCAGATATTACTTGGGAAGTACAGTACTTAGCAGTATCCATCTCAGGATGCTCCTGGTCAAAGGCCAGATAG
ACTTCCCTCTCCAGAATATTAGAAGTTTGCTAAGATATTTCTCCTATTAATTATTTCTTCAGTTCAATGTGAAGGTAAGAATTTGAGATTTCCT
TCTTCATGTAAGCATGTATGTTCACATAACATATTTTTATCTGTGAGCTGTGAGTGTGCCAGGGCAGGAAGTCATGTTAATTTTGAGCCTGCCA
ATCACAGTTTTGAAATGTTTTGGTAAGTCAAGTGCACATAAAGGTAAGACAGCACATTCTAAGACACATTTTCATTAAGCCTGAGGAATGGGCA
AAGCCCAGCATTACCTACTGACCAAGCAGATGGGTGAGGTTTTCAGGATGGGAAAAAATGACAGTGGGAGATGTTCTGAAACATGATAAATGTT
TTCATGTTTTTCATGTCTATCTGCTTATAAAGATTAGTTCATAGTAATACTATCTCTTTTTCTTTCTCTCTTCCCCTCAGTTGTATCTTTCTGG
AACAAATGGTTGGTTGAGCCTCAAGTCTCCGCCCAACAACAACCAAACTGTTCTGGGCCAATATCGCCACCTTGTGGTCATGCTTGAGAACTGA
CCTACAGGCTTCCTTCTGGAAGTTAAAACAAACTTTTGGCCTCCTCTGCCATCTTTATCCCATTTTGAGTCCCTAGATACCCCCCAAAAATTAA
ATATGCCTTAAAATTCTTCTTTCTTAATAAGTTTTCATTTTCTGATCTATTTCCATCTCTGACTTCATTGTATTTGAAGGCTAAACATCAAATA
AGATTATTTCTAAAATAGGCTTGATTAGGTAGAAACCCTATTATTTTAACTATTTTTAAATGATATTAAGTCATGCAATTTGTTCTAAATAATG
CTTACAATTTGTTCTGGATTTGAGACAAGGTGCCATGTAGTTCGGGATAGCTTCAAGTTTCCCAGGGAGTTAATGGAGGATGGCCTGGTTCCTC
CTAACCCCTTGTCTCTGCCTCCCACCACACTGCTGGCTCCTTTAGAGGAAATAAAAAACACTAGTCTCCACATACATATGTGTCTCAGGTCTTC
ACCTATTACCACAAATAAATGAAAATGTACAAATATACCTTATATATTTTAATAACTTTGCTTATTAGCTACAGTTATTATAAAGATCTTTACA
CTTGCATCTGCCTTTTTCCATTTAAAATATTGAACATTTCCTTTACCTGACATAAAACCAGGCTGGAATGACAACTTCAGAATTTTTTTTTCCA
ATGGGAAAATAACATTGTACATTCTGAAATGCTACTTTTCCAGTAAATATTAAGATGCAAGGGAAATAACAATGTAGACATCAAATCTGTGACC
CAGAGGCTACCCTGCATATTACACCTCTGCATTAAAAATGCTCAGTTGCTCACAAGGGCACACTTGGCAGCCAAGCCAGGCCTCCCAGTTCCTG
CTGGCCTGAGTTCCTTCACTTCTCCAGAGTCTTTCATTCTAACCTTTTCCTAGATGCTAGAGGCCTTGCCACACTCGGGTCAGGAGGGGCATTC
TCTTAGGACAGCAGGGTGACTATGCTGGTATACCTCTAAAATACATAATGAGAGGGTTTTTTGTTTTCTCCATTTTAAGTAACAACTGAGAAGT
AAGGTAGCTTTTATAAGGGGTTTGAAAATACCATGTTAACATAAGCCATGTTTTGTAAACACTCAAATTGGCCTCGCCCATTATCTGTCAGTAT
CTGGAAGAGTAGGTTGAACCCAGAGCTTTGGGGGGACAGGGGGAATATTCACAAGACTCCATTCATGTTTTATATTTAATCAGCTCAGTGAAGC
ACTTCATCAAGCTCCCTGTTGTTCTCAGTAGTAGACTAGGGACAGAGCTTACACAATATGTGTGAGTCTATTCCTTATATGTAGCATTTCACTG
AAGGAATACCACAGCTAAACAGTTCTAGAAAGAGTCCCTTGTTGTCATGGACAACCTTTTTGGTTGAATAGAAATCTGTCAGATTCCTTATTTA
GCAAGCACCCACTTTGCATTTATCCCCAGAGATATGAGGGCCTTTCCCATGTTTGTATAGAAACCCTTCACATAGCAGGAATCTTAAGTTCCCC
ATGCTGACAGTATAACCTGTTTTTTGCCAAATCATCTTCTCTCTGGTCACTCATCTCCGTTAGACATCACAGACCTTCCAAGTCAAAACATGGC
TGGCAGTGTCTGCATCACAGCCATAAGTCCCAGCTCAGCAAGATGTGTGCACAGCCTGCTGTGTCCCCACTACTTACGCTCCTTGGCCACACCC
ACAGGCATCTCCAGACACAGTATCTTCTTTCTGAAAAACATAAACTTTCAGTCTTTAACTTTCTGATTACTGATCATCCTTCCCGTACTATCTA
TTGACTCTCTCTCTCTCTCACTCGCTCGCTCGCTCGCTCTCTCGCTCTTACACACACACACACACACAATCATATATAATTAAAGCTCCAGTAT
AAATCAGGCCAAGTAGCTTGCACTTCATACTCTATGTCCTTGGCACCTATCCACCTATTTTTCCACATACATTTCTTACCCCCATTGTATCTGA
TTCCCACACTGACCCTGGGACAACTTGTGAGCAACCCTCATGCCTCCCGAGTTACCTTTCTCCTTTAATGCTATCACAGCTGGTGGCAGCACCA
TCTTCTAAGGCAGAAATGCCAAAGCCCTGCCTTCAGGTGCCCTAGTCACCCTAGTGATAGGCCCTCGGGGGCTTTCAGGAATCCAGTAGCCTAG
AAACCACCACCCATCACCTGATTCATAACCTGCTTCCTCCTGACTCTCCACTCTCCCTCATTTTGCAAGCAGCCTGCTAGAGGAGAGCTGCTCC
TGGTATGAGAAGCCTGAATGTTACTGCCCATGCACCCTGCTGTCCTGGCCTTCTCTGCGAAACACCCACATAGGCATTAGTGACTTGTGCCAAG
AAATCCCCCCAGAGGATTGCACATGAACTCTGCTGTTTTTATTCATACAGAAAAATGCTTACAACTGGCATGAGCTTCTCTGCCCTGCATGCAG
TGTGCTCAGGAAAGCAGCATCACAGCCTCCTTTACCTCAGTGGAGTCACGAACATGCTAAGGGGATGGATCATCCCAACACATGTTCTTCCACA
AGGTGATTTTTACCCAGCTGTGGATGCTTCCTGTTTATACACTTTGCTGCAGTTCTGTGTCTTAGCTGTCATGGCTAAAAACTTGCCACATGAG
CTATGATCCTCTTCATGGAATCTTTTACTACAGAGAGAGAGAGAGACAGAGACAGAGAGAGAGAGAGAGAGACAGAGACAGAGAGAGAGAGAGA
GAGAGAGAGAGAGAGAGAGACAGAGAGAGAGAGAATATTGAAAAATAACCAGAGGTTGAGATTTTTGTAGTTTTACTGTGCTGACGTAGTCTAT
GATCTCTTGCTATAACAAAGATTCCACACACTAGTTAGCATATAAACAATGTAAGTTCGTTTAGCTTGTAATTGTGGAGGATAAGAAGCTGAGT
GTGGTGTCTACATCTGGTAAGGGACTTAAGTGGCGAGATGGGACATTGCTCTAGCACAGGCCTGTCTTCCTCTTATTCAGCTTTGCGTTGCTAA
GTGAAAACCCAAAGCAAGGGGTTCCCAGAGGAACGTGTATCTGGTTACGCTCTAGAGGTTCCAGGCAACCCAGCAGGGAAGGCTTTGGTGAGAG
TGGCCAATGATAGTGATGGGAGCATGTGTCAGAGCCAATCACACATCAAAAGTTGGGAAGTAGAAAGTGACCAACCGGGACAATATAGTCCCAC
AGTCTTTTTGAGGGGACATCACTAGCCATCTAAGGACTTCCCACCAGGTCCCACCTCAGAACAGACTCTCACCCTGAGTGAGCCTTTGGGAAGT
GTTTTTCCAAACCATAGCCATGTGAGTGCAGATACTGGTGAATTTGAATCTACCCAAAGATAACCTAAGAATCGCTACTCCAGAGTCCAGTGTC
ATTAATCACCGCTCTGTGAACCGCAGACCTTTCCCCTCTTGCCACATGGATAGGATCTGGCCTCTCAGGGAAGTAAGGACTGCTGATTTGCAAA
GTTTCGCATCAGAGACTTGTACGGAAAAGCTTTTAGAGAATTCCCCATTACAGAATTTCTCAGTGTTGTCCTGGACATGTGCGGAATTCAGCCT
CAGAGTTCATTCTCACTTCCAGGACCTCAGAGCCCATGCTCCGGTTTGTACGTCTCATCCCATTACTAAGCCAGTAAGTCTTTTCTGCAAAGAG
CTAACAGGATAATATTGGCAGTAAGTAGGCACTGGAGACCTCTGTGTGGAGACCTCCCAGATCTAGAGCTAGCAGGTTGCTACCTCTGTGACCA
TAGCAAGTTCTTTGACTGAACCTCTACCACTACAAAATATAGGATGGTAATGGTGTGGCCCTTTTGAGTTGTTGTGAGGTTAAATGGCAAGTGC
CATTAAAGTACCAGAGGGTCTAGGACAAGGTAAGTACAGTAAGCACTGTTGGGATCAGCCAGAGCCTTGGTGGATGTCACTTCCTGTGCCCCAG
TCCACTGCTCCTTTCCTCAGTCCTCTCAGCTGCCCCAGGCTGGCCATGGCTATGAAAGCTCCTGTATTTACAGATGAGGAATGAGGCACAGAGA
AGTCAGATCACCTGCATAAAATCACTGCGCCTTTAAATAAAGGTGCCACCCCTCCAGGCCAGCCATTTTTCTCTTGGTGTCAGAACCTCAAATG
TCCCAGTCAAGAGAAATTTGGGAAATAAGATTCTCTCTCAGGCTGTGAAGCAGGTGGCCTCCTTTAACTCAATATTCTCCATTGTAAAGTAACC
TTAGGTTCCCCTCCAGGGTCGATTTAAACACACACCTGAATTCACCCCAAAATTTGGGAACCAGAAGGATTCGGGAAACCCAGAAGAGCACACT
TCAGACATCCTCTATGGCCATGTCCTGGCTGCTGCTCTGGGGACCAAACTTTGAGAATCCTTGACCAAAAGTGCTCAAATTATTATGTCAAATA
GAGGCTGGAGATCCAGATCAATGGTTAATAGTAATTGACTGTCTTGCAGAGGACCCAGATTTAGTTCTAAGCACCCACATTTGGCGCTTCACAA
AAGCCTGTAGCTCTAGTCCCACAAAGCTGCACACAGTGTGCATACATAGAAGCAGACACTCCCACATACACACAAACGAAAAGCAAATGCCTGC
TTCTTAATTATGTCACAGTCACCTCACTGGTTATTTTACTTAGAGCAGCATACCTCCCTAACTCTTATGCACCTAGAAAAAACACTGAGAAATT
ATGGCTACAGTTTCCACTGCCATTGCATGTGGGATTCACCACTGTACCATTAGTGTTTTGGGAGCATGTTTATGCAATGTGTTGTATTTTCTTT
TCAAATGAGTAGGCAAATAACAAAACTACATGACTTGTGCTGCGACTACAGCAGAGTCAATAGCGGTGTTTGTTTGTTAACCTACAAGCATCTT
CTTCTTTGAAAGACTGTTACCTGTTGCACTCATAAACTATTCCAGCTCACCTCTTAGCAAATGAAGGAGATGTGACGACACCTCCACCTTAGGG
CACAGGCTGGGGTATGCTGCTATAAGATAAAAAAGAAAGCCAATTGCTAAGAAAACGGAACTTGGACAAGGGAGTTGGAATCAGAGGTCCAAAC
AGCAAAGAGCTGTTCCAAATTTGTTGTGCATATGCGTAAGAAAATACTCACCCGTTCAGACCCGCATCTCTCCCTGCATCCCTAAGTGCATCCC
GTAGGTACCATCCACACTCTCTCGAAGCTTGGTGCATGCACCAGTCAGACTGCCCGCCCTGAGGTACAGGCTCAAAACTGCTTAGAAGCTCCAA
CTGTCTATTTGATCCCTTGTTGCTGCTGTGTTGCTATCTTGTGACTTATTTTTGCAGTGACACTGAGTGCCCTCCTGTGTTGTCTCTTTCAGCC
AGTAATGTTAAAGTAGAGACTCAGAGTGATGAAGAGAATGGGCGTGCCTGTGAAATGAATGGGGAAGAATGTGCAGAGGATTTACGAATGCTTG
ATGCCTCGGCAGAGAAAATGAATGGCTCCCACAGGGACCAAGGCAGCTCGGCTTTGTCAGGAGTTGGAGGCATTCGACTTCCTAACGGAAAACT
AAAGTGTGATATCTGTGGGATCGTTTCCATCGGGCCCAATGTGCTCATGGTTCACAAAAGAAGTCATACTGGTAAGGCCTGGGAGTTTTCCCTT
GAGTGTCTCCTGAGGTCCTGTGGGGGCTGAAGGAGCAAAATGTACTGTCAAGGGCTGGGGATGCAAGTTAGTATTGTAGAGCACCTACCTAGCA
CACACAGTCCCTAAGTTCAAAGTGCACAAGGAGGGTGGGAAAAGGAATGTCGTCTTGAAGCATGTTCCCCATAGACTAGCAGCGCGGCACTTGC
TTATAAATAGCTCCCAAGTTGTACTGAGGCACAGCTTTCTTATTCTAGGGCAGTGTCTGAAAGGTCTTCTCAGCATGTACCTAATCAACCCTGT
GAACGAAAGAGAAGGCTAACAGCATGTGTGTGTCAGCTTCTGAGACTCTGATAGTATACTCGCTAGAAAAAAAGAAAAAAAAAATCCCAGCCAC
AGTGCTGACTCTCATGTCCAATCCTTCACAAGACGAGATCAAAAGCTAGAGACACACCAAAACTGACTTCACAAATTTCTCATTGAGAAACTAC
AGAGAATACTTGTTTTAATCTTGTTAACACTTGGGATTCTCTATTCTGAGGCATTAGTGAATCATTAGTGAGCCATTAGCCTAGCAGCAAAATG
GTCTTTTCGGAAAGGGTGTAGGTCAGCATAAAGTGGTTCACCCTCCAGTCTGAGTCTCTGAGTCAGCTAGCCATATTGCCACAAGTTTTCATCA
CAGAGCAGTAGGGGACTTTCTCTCAAACTCCTATTACCGTAGAAGGTTTGCACAGGCTTTAAGACAATAAGAGAATGCAATAGTCTATTCAAAA
CTAAGAAAATGTTCAAGAGCCATGCCTACCCCTGCCACTGGCACTGTGGGAAGCCTAACCAGCTTCAGTCGGCTCTTCTACTCCTGTTCCACAT
GTATATGAACAGTCAACTCCTTGTTCAACCTAACTGTTACTCCACCACCACTAAGGACACTAAAGAGGCTTCCTAGAAACATCAGTGGTGTTCA
GGACAAGTGTTTGCATGGTATAAGGTTTCTTTGAGAAAAAAACTAAAGACAAACATTACTCTTCTTTCCATAGAAGTTTCTTGAAAGGTAGATA
TTATAACCTGGCAAATTCACCTAACTCTTATAGCCACAGAAAGCTCACATGTAAAACGTATAGAGCCTGGGCTATCACTACCCATACACTGGCT
TCTTTGGGCCCTGAGAAGGCTGGCATCTGCTAGTGCAGTGTCCAGGAGGGAGAACAACACGCACAGATGAATGATAGCGAGTTTTACTAGACTA
TCTCAAGAAGCTGAGGAAGATGGCTTGCAAAGGACAACCTTGTCTGTTTCTTACACGGGACTCAACAACCGTGCGTTATCCAAAAAGGAATCAG
GAGCTAGAACAGGTATAGAGCAGTAGTGTCATCACCAAGCAATGAAGGCGTTTCCAAGGTTGGGTTTTATATATGCACAGAAGCAGTTTCTAAA
ATTATATATTCCACGTATAGTATATGTACAAATGATTTATAACATGATACTTAAATAAATGTTTGTCTTCAACAAAAGTTTAGCTCTAAAGTTA
GGCAAATTTGGCTTTTCTGAAAGGAGCCTTTAGTTCTCAAGTGACTCGTAGAAATAGACCTGGCATACCCAGCCCTGAGAAAACCAAATGTGAA
GGTAACCTTGCTCTTGCCTTCTGTCACGCAGTGGAAATTAACATGGAGAACCCTGTGACAGATGCTCAACCAGTCTAGGGCCTGTAGCAAACCA
TTGCTGATAGAGAGCAAACCTCTGACTGTACAGTAAAAATCCCCTTACATATTTGACAAATGAATCAAATTCTCGTGTAGTAAATTGCTAACAT
ATGACTGCCTGCCTTGACTTAAAAAAAAAGAAGAAAAATTACATGTAATGATTAAACCTGCTATCAACAGATCTGGGAGGGTACCTGATATTTG
CTACATACTTTCCTCAACAAATAGTGACTAAGTATCATACCTACCCCAATATGCTAAGTTCTTCAATTATGGAACAGATAACTTACAGAAAGAG
GAAACAGGAAGAGACTACACACACGCACGCGTGCACTCATAGACAAAGTTTGGTCATTGCCAACTCCACAGTATATCTCCGTATATGTGAATAT
ACAAATCCCCAGCTAGAAGTAAAAACCCCATTTAGCCAACTAGGTTTGAATAGCTAGACAGAGCAGATGGCCTTTCTCTTCCAGGTCTGGGTGG
AATGTGTCAGCAAAGTTTTGGGCTCTGCATTCCTACAATAACAGCAGGTTATGAGAACACTGCCCACCCCTGTGAAAGGCGCTGGGAATCACTT
GGAATCTTGAGAACATGCAGGGCCTAATTCAGACTCTGGGAGAATCTGGAGAGCCAGCATTCCTATGAAGTCCTCGGAACGCTGGGCATGGAAG
CTAGAGCACAATAAGCCCCCGGGGACATCCTGCTGAGGACAGGACAATAAGCAGAGCCATCTCTGTGGCCTATGAGGAGCTGATGTACGATGTG
CTGCCCCAGTGGACAGCTAGGATGTGCACCAGAGCCTCTCTCTCTTCTTCCCATTGCCCATGACCCAGTCAACCAGCCTCATGACTTGCAGCCC
CTTTCTAGTAAACAAAGATCTCAGGGGCATAGCCCAGCTTCTACTCCTCTCACTGCGTGGCATTCCACTGCCGCCATTTTCTAAATGGCTTGTG
GGCACCGGTGTCTCACTTCCCCTTCGGTCCTCAGTTGGTTTCATGTTTGATTCAAGTGTCACAACCCCAGCGTAGCCACCAACAGCTCAGGGTG
CATCTGAGGATCACCCTGGGGTGGTAGGGAGCTGCAGAAGGGCAGCGGGGCCCACCTGTAACATCATTATTCTGAATAGCAAAGACTAGGAAGT
GGCCTGAGCAGAACAAACAGGCTGGCTTCCTGTCGTCTCCTGCCTAGCAGGTGCTGCCACAGGCCTGGTAGTTACACATCTATTCTTGTATGCC
AGGCTTTTTTTAAAATCTACCTTCTCCAGACAGGCCTTCGCATCTTCTACCAGCTCTTTTTCTTTTTTTAAAGAAGATATTCTTTATTACCCAA
GTAACAGGTCGCAAACGTTGTGACAGAATAAAAAGTGTGCAGTTCCTGGGATGGAAATTAAATTCAAAATTTGCAGTATCTAATGTCTCATCCC
AGTTAAATGTAGGTGGATACAAGCTTAAAGGACCTTAACTTAGCTACAGGCTGTTGTCCTCCAGGAAACAGGTCATGGCCACTGTCAACAGAAG
CAGACATGGGCACTGTCCCATGATCTTCGAACACACCCCCTTATGTGTAACACCTGCACTGAGTGAAATACCATATGATCCATTTTAGAAATGT
GGTCTTTAAGCCTCCCCACCATGGGCTGTACATTCATGGCCCACACAATCAATTGGAAATCAGTAGATTTACAGTAGCTCTGCCTTACCTTGCT
TTGTAGCAAGGCTTGAGACACCAGGCAAGCAGTCCCCATGGAGAGATTTTGACTCAAGTGAGAAGGCAGGTTTTAGTCCCCAAAGAATGCCCAC
CTTCCCTTCCTGTAGAAGAACAATGTGCTTTGAATTATCAAATTCAAAGAAATTGAAAGAAATTGATAATTCAAAGGCATTATTAGTCCTGTCT
GCCTCCCAATGGCCAGAACACTAGCTCGAATCACATCAGGGAGACACCTGGAGCGCCTCCCAGCTCAGAAAGGGGTCACTCAGCTGCTAAGCAC
GTGACTTGGCCCACTTGGATCACCAGGAGTGTCTTCTGAAGCTGTCCCGTGTGCTTTCCCATGCAGCCTGAGGGCCCAGCCCTCTCTGAGGAGC
AGCTACAGTGTTACGCACGTTCCACTTCCCCAGCCGGATCTTCCTGCATACACTGCTTACTTTTCTTGTTTCCGCTCCTCCAGTTGGAGCTGAA
CACTTTGTGAACACAGTGTCCGCTGAGTGTGCACTTGGGTTTCCCAGGCGAGGTGATGGTTAGTGAGGCTTCTAAATGCTAACACCTACAGTTA
TCGTTCTCCATCCTCCTTGACGTTAAACAGTGTCGTCACGTTCACCTCGGTCTAATCCAGACAGGAATTTGGGGAAACGTTGAATACTTACTGG
TTAGTATCAAGAGCCCTCTGACTGGCCATTAAATATCATTGTAAATTAATTCTAACTTTTGTGTTTGCAATGCCCAGTGAAAAAGACCTAGAGG
TAATTCGGAAATTCAGGCACCAGTATGCACATTAAAGCTTAACCTGGCACAGCTTTTAACAGTACCACCTCTGATAAAAAAAACTCTCTGCTGC
CCCTCCATTTCCGTGTTGGAGAAATGTTAATTCGGTATTTTTAACACATTGTTCACCCTTAAGAGAAATGTGTCTCGGGACATGGGTTTCATAC
ATAATGGTCTTTTAAATGAAGCATCTCTCCTTCAAAGGCAGCGTTTATAAGGCACATAATGGACACACCATTCCTGAGAGTTAAAGGCTAAGTT
AGTGTCCACCCAGCACGACAGTCACACACTCTTTACTCACAAGTAGCTCCTGCAACCATCTCCAAAGCCAGGGTGTAAGCCAAAGCTCTTGGGC
ACATAGTGCACGAACCCTGGGCTGTCTCAGTCACAAACCAAGAAACCTCCTTGGAAACGCCCTCAATGGCAGCAAGATCTGTACCTCGAACATG
ACTTGTCTACTGTGGACACAAAAATGAATTCGAAGGATCTCTGAGTTGTGAGGAAAGGGAAAGGCAGGAAGTCTCAGGCAACTCCGAGCGCGGG
AGCCAGGGGTTCCCTCTGGAGGACACGTCACGGCTGTCCTGTCCCTGAACTTCTCTTTTATTCAGTCTTTCTCCAGTCTCTCCCTCTCACGCTC
ACACTTTCTCTGTCTGTCTCTCTCCTGATTTGCCTGCTTAGGGACAGACAGGTAATGGAGAAGAAATTGAAAGGGAAAATACAGGGAAAATGTA
AATGAAAAGAAGTTATTATAACTGTTATAATGAAGTGCCTTTCATTTAAATATAAGAATGTAACGCTGAAATGAACTTGTGATCCTGAAATGCA
TTTTTAATGAGTTTCCTTTTTATTTTGCTGCTTCAGTCGCATATTTTAAAGACCCTTTGAAAAAAGCCACATTAAAAACCCCACCAAATGCCAC
AACACCCAAAATTGGATATTGGTTTATTCCAAAACTGCTGATCAGGAAGAGTGACTCTTCATCACAAAATGTTGCAGTCACTTTATTTAAATGA
GTTTGAATTTGCATTGTGATGTGCCATTCTGATTTAGGGAAAAAAAATTAGATTTTCAGATCAAATTGACCCAGCCAGTGAAGCGTTAAGCAGC
TGGCAGGTTTAGTCTCAAAGCCTCATGTTCTATCAAACCTGCAGCCGGTGGAAGTCACCAAGCCCATGGGAAACAACTTTCTCGAGGCCGCGCT
CCTGAGCAGCTCTCTCTCACTGCTTACCATGCTACCCTCTTCCCTTCTTGCCCAGGTGAACGGCCTTTCCAGTGCAACCAGTGTGGGGCCTCCT
TTACCCAGAAAGGCAACCTCCTGCGGCACATCAACCTCCACTCCGGTGAGAACCCCTTCAAATGCCATCTTTGCAACTATGCCTGCCGCCGGAG
GGACGCCCTCACCGGCCACCTGAGGACGCACTCCCGTATGTTCACTGAGCATGGCAGGGATGCCATGTAGGGACCCCGATCATGTGTTCCATGT
TCCCACCCACTGCCCTTCCAATCCACCCAGTCCACAGCCCTCTGGGGTGTTGGCCTCCTATGTGGTAATCCCTTATTCCTACTGAACCAGAGGA
AATGGGGGGGGGGGGTGCACACATGGAAACCCTGCCTCCTTGGTGTGTTTTACAAGAGCTTTCCCTCCCACCATGTCAGGTAAGTGGGTATTTT
TCTGAAGTATACTGCTCATGCCCTTAACCTTCCATACACATCCTTTTACACCTATTGCTGTCTCACTCATATCACTGGCTTATGTGGAATATAT
TGACTAGCAGTAATAAACGTAATTATTTTCGTTGCAGAGAGATAGGTTGGTCAGAATATAACCCTCTCACCCCAGCGCACACTGCCCCAGCCAG
AGAGTAACTTCCAGGCTGGAAACCAGCAGAGGCTTTGATCGGACTAATCAGTCTAGAGTTGTCAGCAAACTGCAGACAGGAAAATTTCCCTGTA
AGGATGGTCCAGATACTCTAGAGTCTCAGAAGACCATTTATATAAATGCTGGACAGTTGTCAGAATGTTCCCAACCTGTCACACAGACTCATCT
TTAGTTATCATACATCTTCTGCCTCCTGGGTGGCATCTTATCCAGTAGCCCTATAAGCCTCCAGGTCTGAGATATAGGAAATAAAAACTATATG
CTATGTTTGCTTGCAATCTAGTATTTGTAACATGCATATGTCGCTTCCATTCTGCACTGTCGTGTGGTGTCACGTGCTACACTGGCTTTAGGAG
CTCTAGGTTGAGGGGCCTTGCCCCTGCCCTCCTACAGTGCCCTGTCATAACAAGCTGTCACTCTGTCCTTGCTCAGTGACAGAAGAAAGGTCCT
CAGCTCATCTTCCCAAACAAGATCTCAAGTTATCCATCTCTTCGCCCCATATTTCCTAAGCCCCTCAACAGCGTCCTATGTGTGTGTGGCATGG
TAGAGTCCCGAGGGTCCTGCAGGAGCAGAAAGAAAAGACAGTCTGTCCTGCCTGGAGGATCCATAGAGATTCCCACAGCAGGAGCGCTACAGGG
CGGGGCCAGCTCAAGCTAAGTTTGCTTTAGAGGAACTCTAAACCAATGAGAGCAGAGGTTATTGCAGCACACATTTTAACACGTGGTATCCAGA
GCTGGGCTTTCTGGTTAGTGGTCACCTCCAGGTGGGCAGATGTAGAGAAGAAAAGAAGCTAAGGCTATGTGTCATCTCCCCTGTACTCTACTTC
CCTACCTGACTGGCAGTAAGCAGTCAGGAGACTCCTAAAAGTGTGCAGCAGAAAGTTGTGACTTGGGCGAGTTTCTTGGCACTGCCAAGAAAGG
TCGTCCCCTCCGTTGAGGAGCTGTCCTCTCAGGCAGCATCTCCGTCACAGGACGTGTCAGGAGACAGACACCTCCCTGTCTTAGGTAGGACACT
ACCTCAGATCTGGGAGGCGTTTTCTACAAGGAATGAGGAGACCCAAAGGCTTCCTGGGATGGAGGTAACTCTGTCTGTGTGTAGCTGCTCCACA
AGGTGAACGCACTCCAAGGCAGGGAGTGACATACAGACCTGGGTACGTCGCACCTGACTTAGGAGAAAGTCACAGAGACACAGTAGGCAGGTAC
CATGCACTCAGATTGGCAGCAAGCACGACTTCCCAAGCCAATGGAAAATTCCAAGACCCACTTTTACCCTGATACCACTCACAGCACTATCACC
CTTTAATTACTGGTTTACAGTATAAATCTTTCCTGCATCCCCATCCTTCAACTTAAGATAGCAAGATAACATCTTGTGAAATCAGTGCTGAGTT
TTGCGTTTTGCCTACATGCTTGATCATTGCATTTAAGGCATGGTGGTATCCTCCTCTCGGTGGGGCTAATGATTTGAATAATTATTTAAAACAG
ACAGAGGGCTCCTGACAGTTATTTCTGCCACAGAGTCACCAATCTCTCGTAAACATCTCCACCAGAGACCTGGGGCATTGGTGACTCGATTGTC
TGAAAATGTACTTAACAAACACAGCTCTGGGAGAAAAGAAACACTGTGTTTCCTTCTTCAAAACAATTTTACCAGAATGACAGGACTTCAGCCT
TTGTCTGCAAAGTCCAGGGTGTTATATTTTCTTAATGAATGGCCACGCCCACATTCCATTAGAAGCTAGGGGCAACCATAGCTTTCCCTAGCAT
CCTGTGGTGGTCTGACGTTCTAGACTCTACTGTGATGATGGAATTTGTGCTGGTATCAGGGTTGGTACTCTTTTGAAAACTACAAACTTTGTTA
CAGTCAGTTTGGTTCTCGTGTTCTAGCATTAAAGTCAATCATATACTAAGAAAAATCAGGTACCATACCATTTATATAGGGAGGCAATTTGATT
CACGAAATCACTAAAATTAAATTTTTTTCGGGAAAGGGCATGTATTAATTTCTGTAACCATAGAATATAAATGTGCAACCATATGTAATATTCG
TTCCATATTTCATCATTTGTGGTACATCACTGTCTCTAGACAAAAATTCTATATAACTGGAGAGACGGCCCAGCAGTTGGAGCACTGGCTGCTC
TTCCAGAGGACCCACATTTCATTCTAGCACCCACGTGGTGATTCATAACAATCTGTAAAGCCAGTTCCAGGGACTCCAACTCCCTCTTACAGCC
TTTGTGGGCAGACACCAGGCAAGCAAGTGGTACACAGACATACAGGCAGGAAAAAAACCCATACCCGTGAAATTAAGTAAAATAAAAATCTGTT
CTGGAGTCTATAGATGAAGCTTTCGTTTCCTTGAGAGCATCTTCATAGCATCTGGTCACTTAGGAAAGAGACTTAAGTCACACTGAATTACCAG
TTATCAAGCATTCCTGTTTGGATGGCTTTATATCATACTTAGCATGAGAAATGCAATGTGATGTTGAGGTGAAACTCTATATTCCACATAAAGT
AATTGGAGACCATCTTCTCTAAAAAAGGGTCTTTATTGAAGTCTTTAAAAACCGATATTATATGGACCCTTTCCTATTCTTCTAAAATACCTCT
CAAAAAAACCAGGAAGGACTAAACTCTTGTAGAACCACATTGCCACACATAAAAGCCACTGGCCATACTTAAGTTTTAGTAAATTAAAATTAAA
CAAAATTAAAATGTTAGTCCTAACATTAATCTAGCCAAACTTAGAAACTCCATGTAACTGGTAGCTACCATAGTAAATAATACAGTGAATGCCA
AGCTGAACAGATATGCAAAGAACACTTAGAGTGCTCAAGTCGTTAGCATGAGTTCTAATGGCCAAGGCTGATCTGGGAGTTTCATCATCTCATA
TTCTTTCGGCATAGAATAGAGGCTTTTCATATCCTCTTCCTATGCAAAACACATTGTATATCCTCCAGGTATATGAAAATGAATTTGGCTTATC
CATATCCCTTTTCATTTTGTGTTTATATATTGTATATTCTTAATATTCTTAAGTGGGATAGTATGTACTATAGTGTAAAATATATTTGTATTAT
ATAGAAAGATACAATTTAGAAATACTCATTTCTTTAAAGCCTTTTCCTGCCATGATTTCTTAGAGACTTGCGCAACCTCATTTTGCCTCCTGAA
TTTTCACCTTATTCATTAGAGGTTGTCACAGTAAGGGGTCAGCCTGGGGAGAGGCCCACCCTGACAGTGAATCTGTACTCTCCTCATGTTTCTG
TCCACACTGGAACATGGAGATAGTTTACTTTTTACCATTAAAGGAGACCCTTGAGTCCGTGTCATAGGTTAAGATCAAATTCATGAATTCTTGG
TACACTCAATGAGTCACCTGGGCATTAACATTTTTAAAATTCATTAATTTTGACTAAGTCCATAATAGTCATAGCTCAGTAGAGAGTACATTAT
TTTTTTATCTTTTTTTTTTGGTTATAAATGTATTGATTGCATCCCCATTACCCAGAAGGCCAATATTTAATTGGAGTCTTAAACTCAATTGTGT
TTTCTGCACTTGGTAAGCCTCACAAATCTGCATATTGTGGCCGGAGCTATAAACAGCGAAGCTCTTTAGAGGAGCATAAAGAGCGATGCCACAA
CTACTTGGAAAGCATGGGCCTTCCGGGCATGTACCCAGGTAACCACTGAGGCCCTGCTGAGCTGCACCCCTCCCCCTCCCACCGCCTGGCCCAG
GATGGGGCTCTGTGGCCTGTTTCAGCCACAGGAGGAACTTTTTGTCTTCTAGCAATGCATCTACACTAGGGACAAACCGTGCTTCTCTACTTCC
CAAGTTCTCAGTTGACCAGTCATACATAACCTGTCTTAAGACCTCCTTAACAGCAGTGACCCTTTTAAGAGTCTGTGTTGTGGGGTATTTTGTC
GTTGTTGTTTGCGTTGTTTGGTGTGTTTTTTTTCCCCAGGGGGTGGTGGGTGGGATTCAAACCAAGAATTGGCTAAAGCTATAGCTAAGTGATG
AATGAGCTTGCCTGGTGTGCAAGACGCCCTGAGTTTGATCTCCAGCACCACAAGTGAGAAAATAAAATAACCAAGTTAGAGTAGTGTCGTTCGT
GATTTCTTCTTTGTGTTTAAATATCCAAATAAAATGTGAGTCTTTTAAAAAAAACACTAATTACTCATGAAGGTCTTCAAAAAAAGAACTGTTA
AGCTAGAACTAAACATTGATTTAGACTGAGCTTTGAGTATCCACAGTTGGAGAGAGCAGAACTTGTTAGGAGACCTGGTTGGCACTGGTTCTGA
GCCTGAGGAAGAGTGTTGAATGTATATTGAGTACTACATTGTCGTGAGAATCAGTTTCTAATTTGTCCTGCCTGGTGTCTGTTGATTCAGAGCA
CTGCAGTGATGGCTTCATGCTCCAGTTCTTTCTCAAGAAGTGAGAAATTACTCTTGCAAGAAGCAGCAATCTGACCTCAGCATGCCCAAAGAGG
CATTTAATTACATTGGGTTCTTCCTGCCTCTGCCTCAGGGCACATTAAGATACACTAGATGATTATAGTTGCCCCCAAGCCAGTGCTACCCAGC
TTCCATCACTGTAATGGGTGTCTGAGACAACCGGTAATAATAGGAAGGAAATTCTGTTTTGCAACTTTGGAGGTTTCAAACCATGGTCAATTGG
CTCAATTGCTTTGGCCAGGTCATGCTGGGAGCACAGGTACACTGAAATCTCCTGCCAGAGAGACCCTAATAAGCCAGGGAGAAAGAGAGAAAGA
AAAGGTTAGGGACCCATGCTCCTTGTTGAGAGCATGTCCCAATGATTTAAGGACTTCCCCCAAGACCCCAGCCCTCAAGGTTCCCAGCATCTTT
TCCTTGGTTCATCCTAGAGCCAAGGCCTCCCACATGTGGGCCTGTGGGGGACCCTGCAGTTGCAAACTGTGGTTGCCTAGGTCATGTCTTACTT
CACTATTTCACCCCTGAAACCCCTGCCTTATACATATACACTGAGTGCTGGCTTAGGGAATATGGCATTGAGGCTCCATCCTGCCAATAAACAT
GTACATTCTCATTTGTCTCCTGACCCTGACCCTCCATCTCCCTCAGGTCACCTCATTACTCCCACGGACCTAGTTAGGTTGGGTCCATTAGTTG
AAAACTGCCTAACTCAAGAGATGTAAGCTCAAGAGATGTAAAAGTAAGGTTTGTTTTTCTGAATATGATTTTCTTTAGTAAAAAGGTTAAGGAT
ACCTGTTACATTTCACCTATATCTGCATAGGATAAGGAACTATCTCCTGAGCCTCACATGCACCAAGGACAGAGGAAAAAGTACTGGGCTCAGA
GGTGTGGTATGATGTCACCTGAGAGATGGAGCACCTGAGAGAAGGGAAGATATGTAAATTAGCTTGATAAATCACATGTATGAATGACTTCTAG
AATTAGATTTTATCACTTTCTGTATTTCCTCCTATTAATCAGAGTGGGCTGAATGTTGAGCTATTTAACAAGACACCCTCATAATTCACTGTTA
ACAGAATATGCCAGCAGTAGTATTTGACTAGAAATGCAGGTTTTAAAGTGACGATATAAAAAATGCATAAGGATTGTGTGGGCATGCAGGGTAC
AGAATACCCAAGTGGCAGAAACTACCAGGTCATCCTGGGCAAGGGGGAGATGTAGAAGATGATGGAGTGAATGAAGGCTGAGCCATTGTGAATA
TCCATTTATTGTAAAAAAAAATCTATCAGAAAAAAATATATTTAAATATTGACAAGACTAGGATTATGTGAAGATGTAGCTATGGGTCAAGGAA
CTCTTTGAGGAGCATGAAGATAAACTAGATAGAACCAGATAAACCTATGGTAAATCAATGGCCTGAGAAGGGAAACACACAAGGCAGGAACAGT
ATGGGAAGAGCTGTTTAAAAAGTAGAAGCTGATCACTTTACACTCTAAGCTCCTGGGGAATGAAGGAATTATATATTTTAGAAGTCCCTGCCTT
GCAAACTAGGTGTCAGGATAGAAGAGACTATGCAGGAGACAAACTTGTATCTTCTTCACTTATGGGCAGGGAGAGTTCGTATAGAAAAGCAGAT
GGAATGAGCTAACCTGTATGTCTGTGTTTACCTTAATGGCTGCTCGGCTTCCTACCCTGTGTGATTGCTGTGTGGTCATTAGACATGTGCTGTG
TGGTTGGGGACTGCTCCCTATAGAGTTGTGACTTTAGACCCTTTGTTCATTTCTGGACCAGATGATATTTGGTTCTCATTGTTGTTGTTAAATT
TGGTTAAGGCAATCTTGACAAAGACCCTCCAAAGTAACACAGTTAATTCACTGTGGTCCAACCTTGTGTCGTAGACAGCAGCTTGTTATTTTGG
TCTTAGGGATAATGTGGGTTGAACAGGTGGAGGATGGGGTCCTAGCAGGTGGCTAGTCATTGGCATTGCTGTAGCTGGTTCTGCCAGGGGCTGC
AGGATGAGAAATCGGCTCTTATTGGCACTTGACAAATCTGCAGTTAACACTGAATACAGCAAGATCTTGAAGTGCAAGCATTTCTAAACTAGCC
TCTCTAACTGTCTTTGACTTTAGTCATTAAGGAAGAAACTAACCACAACGAGATGGCAGAAGACCTGTGCAAGATAGGAGCAGAGAGGTCCCTT
GTCCTGGACAGGCTGGCAAGCAATGTCGCCAAACGTAAGAGCTCTATGCCTCAGAAATTTCTTGGTAAGAGTTAAATGTTTGCTGTCTCTTAAC
CTAGAATTATGTGGGTGTTCTAGACAAAAATTATTTGAAAGTAGAAGAAAACAAAAAAAAAATCTCCCTTTTGGAAAAAAAAAAAGCTTATTAT
TTCTTAAATACTGTAAATTTTTTCTAATAATATTATTCAAACTGATAGACCTTTTACCAGGAATTAGATAGGGTGTCATAGCATTTGGAGCCTT
TGGTATCATGTTTCTGTGGCCGCTCTGACATGGTTACATCAACTCTTTCTCCCCTCCAAGGACAGTGAGGGCTAATAAGAACCAAACAGAGATG
AGCCCCCTCAGAAGACAGCGGAAGGGTACAGCTAGACTTGCTGTTCTGATCACTGAATCCTTACCTGTCCTTGATACTATGCGGAATAATGCAG
AGGGAATGAGTTGTTCATGAGGCACTGTTGCCTGCCAGCTAAGGCGGACACTGTAAGGACAAGGAAGTAGCTGAGACAGATGCCTGAATGGCAT
CACTAGCCTCAAAGGCAAGCTTTACGTCAGAACAGAGATGCTCGTAAACTCTATGGGGGCAACATGGGGGAAATGACCCAACCTTCCCAAGCCT
GGGTTCCATTCTTTATGTGTCTCATAAGGCAACCGGAAGCATAAAATGCCCCCACAGATATAAAAAAAATTTAGAAGCCCTCAGTAGGAAGATC
AGCTACTATCATTGTCAAGAAAAGAAACCTTATGTCCAAGAAAAAAAAAATAAGGTGTTTGAGAAGCTGAGGTGGACTTTCAGAAAGGGGCAGG
ACCTCAGCAGCATGCCATACAGTGAAGGGGTCATGGAGTCTAGGCAGACAAGGAAAATTACTTTGCTGGTGTTGGGATCTAGAGCAGGACTAGT
GGGTATTTCCCAGCTTTGCGCAAGTACTCACTGGAAAGCAGTGAGTAAAGCTGTCCCATGATGCTCTGACTATAACAGTACTGGTCATTGAGGG
CCAAACTGGGCAGCATCATCTCATTTGGAATCACAGAGAAAGCAGGGGTATGCTCCCAATGGGAGCACAAGCTGAGCACATAAAGATGACATGC
GTCAAAATTATCTGGACATGGGAGATGGCAGGAGAATGGGGAGTAATTGAAAGAATGCTTTGGTGTACAAGGAATGGAGGTCACGGTAGGGCTC
GTGAAGGAAACTGTGACTTCCCTTGCTTACTGTGCCATTTGCAGAAAAGACAAAGCCACCTCTGGAAAATAAGAGTTCAGGGTTTGATGTAATT
GCTTTTAAGAATGCTACCCTGGTAAGGCTACCCCATGGAAGCTGTGCTACAAAGGATGTGCAAATAAAACGTTGGTGACACACACACACCTTTA
CAGAGCACCTACACTGAGGCAGAAAGACAGAGGTCAGGTTCTAGGAGTTGATAAGAAGGAGGGGAAGAAAGACCACTAGAAAACAAAACAGAAA
CCACTTATTAGAAATATTGTTCATACACTTGTCTCTCTGTGTATCAGCTTTTGAACCAAAAGAATGAAGGAGACTTCATATCCTGATCAAAGAA
AGAGGCAATCTTCGTCAAGGAGCCACCTCTTGGATGGCTGGGGCTTTAGAAAGTATGAGGTGCAGGGCCCTATCTTCTCCCTGAGGAGTGCACA
GAAGACAGGGACCATGATGAACTATCTGGGAGACTGGCAGAGATGAAGCAGCAAATGTGTTCAAGGGTTGTGAAGAGTAACTGACCTCAGGAGG
CAGGGTGTGACTTCCTCTTTCCAATTAGCAACCTGAACAGTGCCACATGCCTTAGTAATCAGCATCGGCAGTGCCCTGAAGCTTCAGGAGACTT
CCAGCCAAACAGAGCATCTGAAACTTTGTCATGGCTCTCCTGGCTGAATGGGCATTGTCTTGATTCACACGCTCTATGTGAAAGGGATCTACAA
AACTAGTGAAGCAATGCTGAGAGGGGAAAGCCATCTCCAGGGGAGAGGTTAAACACAAGAGGAAGTGGCCTTGTGGGAACAGGAGGACCAGGAT
AGCAGAAGTGAAGTGTGCAAGGAAGGGCTCCCAGCAGCTGTGCTCTGTTGGTGTCCCCCAGAGGTGGTTGGCAGTTCACACACTAACCAGTGCC
TCTTCCTGCTCTCGTGTCTGCCTTCCTCTCAGTGCTACATCACGACATTCCTGTCCACCCTTCCTACTGTGTCACAGTTTTCTCCCTAAAATGA
AGAGAATTGGACCTCAAAGGCTGCTCCCCACATTTAGGATGATATCGTTTTGTGTACTTGAGATGATAGATAAATAGATAGTAAGAGTTTTGAT
AGAACTATTTCATGTTTTTTCAGAAAACCAAATGCAACATCCAGAAATTACCAGTATTAAATTAGAAGAACATCATTGTAAATATCTTCCTGCC
ATATATATTTCAGTGGACAGGATTAATAATAGGTGGACTACAAATAGAGATGTGGAGGAAAGGCCTTTAAGTTCCTGCGTGCGCCACCTCAGAA
AGATTGCACCTGCTCTTCCCTCGGCTGGAGGTGTTGCCTGGCTTCCCCTCTTCCCCTGTATTGATTGAAAATCAGTTCACAGTGGCCACAGCAT
ATTCAACATGTCCTAGGAGATAAAGGCTCCTCAATGCACAGTCATTCCCAACAGGACTTGACTTTTTTAAAAGGATGACTATCAGGGAAAAAAA
ATTATTCTAAAAATTACTTTAAAAACAATCATTTTTATTGCTTTTATTTTTACATTGTACATTTCTATTCATATTCTATGCAGAAATCATTATT
CTTATATAATTTAGATATTCATCACTATATATTTTAACAGGTTACAGAATCAGACCCTGGTCTCTTAGGCTCATTTTCGTTTATTTTGATCAAT
CCCTTCATTGAATACACTTTACATTCAAGCACCCTTGTAGACATTCCATCTCCATCTCTGCTTTGAGTTTGAGGATGTCACCTGTCACCTCTCT
ACAGTAACCCCAGGAGTACGTAGCCTCTGGCCACTCTTCCTTCTCTGACATTCGCACATTTTTCTTTGTTGAATTATGTGGGTGCCAAGTACAA
CCTGACTTTCTTTTCTTTGATATTACTTTTCTCCCAAAATATGTAGTCTTCTTTCCTTGGGCCTAAGTCAACCATCATCAGTAAGAAGCTCTTT
GGTGTTTTACAGTCTACATGAAATGTAGCCTTTAAGAGTGAGAGGCTCCTTTCTTCCTCCTTGTCTTTTGGAAACATTCCTTTACATAAGTACC
AATTGTTCTATCTAACATGACTTCTATTCAAGAGTGCTGGTTTTCCATAGACTGGATAAATTGTCTCCCATCTCTGTTAGCATTTTGCTATTTT
AATTTCTTTATTTTTTTAAGTTTACTTACTGTTATTTTATATTTTTCCTGATTTAATTGAGTTCCTAGCAATGTTTATTCTAGTGTTTGCTATT
TTTAACTCATGTATTGAGTATGTATTGAATTGGATTTGGTTTTAATTCCTGAGATCTAAAATCTCCCTTTATTCTGGGTTGTTTTTTTTTTTTT
TTTTTTTTTTTTTTTTTTTTTTTTTTTAGTATTGTCTTTGAATAGCTTCAAAATCTGACTGCTTGCATATTCTAAAAACTAAAATAGCTAAAAC
TTGAAAAAAAAATTTTACCCCATGTCATCAAGTAATTATGCTTCATTGCTCCTCTTCCTGGAATTAAGTTTCTTTTTCCCCTAGGATGGACTTT
CTGGTTTGTCTTACTTCCCTTCTTGCCAAAAAAATATTTTCCCTCTAACCATCGCATTTCTTTTCATCCTGCTCATTCTACTTGGTCTAATGAG
GCTTGACTGACATTTCAACATTCTTCCCATTGTACCTGAGACTTTTCCTATGAGAACTATAAGTTTTTTAAATCCTGAAAGAATGCCAAGGATG
GTGTTGGGCGAGTTCAGGTGAGGCCAAGTACATACGGCTTCTGTTTAAAGTGTTCCCCTTTGAGAGCTCTCTCAGAGTTTTCAGATTTGGTGAA
AGCTCTAAACCAGCAGATGATGCCTTCTGTCTCCACAAGGGACATCTCAGGGCCTACTTCCCCTGAAGTACAACTCACCAAGGCCTTTGCTTGT
GTGCAGGGGAGCCAGCCCACATGCTAAGGTCTCTGGTGTCATGTGCTGCCTGAAAGTGGAATTTTCAGCTCCATCATCAAAATGAGGAGGGCAT
TTGCCCATTGGAACTCTGTCAGTGTGAGGGTTTGGAAGGAATGCTGAGAGTGTTGTGTTTCTAGAAGTGACATTACCCAAAGCTTTGTCCAGTC
TCAGGCAAGAATCTTCTGTACCTTTCCTTGGTCATATGTAGTTGAATGTAAAGTAGAAATAAGAAGATGGATGGATGGATGGATGGATGGAATA
GATAGAGATAGATAGATAGATAGATAGATAGATAGATAGATAGATAGATAGGATGGATAGATAGTAGATAGATAGGAGGGAGACAGGGAAAGAA
AGGGATGGATGAGTAGATGGGATGGATAGATAGATAGATAGATAGATAGATAGATAGATAGATAGATAGACAGACAGACAGACAGACAGACAGA
CAGACACACAGACAGATAGAATAATAGATTTCACTGGGGCACTGGAGGCTTTTATAGCTTTTAATTTTTCACATTTGGGTAGAGAAATTCTACA
GGTAGATTTGTCACCCCATGCTCATCTGGAATGTATATTGCTTTCTTTGCAACACCCACTTCTTGAAGCAGCAGGCTTCAGTCGTTGCTTAGAA
AACCACAGAACCACAGCTCTGGGGAGAAACAGGAAAGTACCACCTCTGGTGAGAGAACACAGACTTTTACAGCTAACAGAACTGGGCACCAGGC
CACACCCTCCTCCTCCATTGCTAGGGTTTGGTAGTAAATGAGATGCAAGTACAAGAATGAGTTCCTCATGTCCACATAATGAGATGGGTTCTCA
CCAAAACCACAAAAACAAACAAACAAACAAACAAACAAATAAACAAAACTCCCCCTTGAGTGGAGAGAGGAATCCCTTCATGGTTTCTACCATC
CACTACACGTGGCTTTTGCTCTGTCCAGTTTCTGAGAGTGTGTAGCTGCACATTAACACGTTTTCTTCCTGTAGCCTGACGATCATTGTATGTC
GTTACCAGAGTATGCGTTGGTTCTGGCATGAGTAGATGAAAGCAAATGAAATCAAGCCATTGTGTGGTGCACACCCAGGAAGATGAAGAAAATG
CAATCAGGAGGATACTGAATCAGGAGGAATATATTCCAGGGCTACTATAGTCACTGATTATTTTTTCAAGGACTATGGTTAGATTTATGAATTG
TGGAATACTTTTCCTACTGTAGATAAAGAGTTTGGGGGAAGTGCTCACCTCAGTGCTGGCATGAAAGACACTGGGATCATCTTCTACCACCACT
CCATCAGCTGCCTTCAGGTACCACCTGATTGAGCAGCCTCTCACCCTGGTAGCTCAGTCTGTTGCCCCACTGTGCCTGGGTAGTTCACTTTCTT
CAGACCCTTCACCAGTCCCCCTTTTTATTTGAATAAAGTCAGTCTAGTCTAAGGTTCCAGTGTATGATCCCAGCTGTACTTGGACATTCCCTAT
GCCCCCAAGAGCTCTGTCCCCTGGACCTTTAGATTGTCACCAAAGCCCCAGGCTCTCTGTCCCCTCGGGATCTTCACTGCTTCTGTTTCCAACA
GTAAGATACAAAAAGCCCCAGCTGGCTCTTATGAGATGAGAAACCTATGCAACACTCTGGTAGCAAGCCTTCCTGAGCTCGTGGGATTTCATAG
ATAGGGATGGAGTCTGCTGTCATCATTTAGTGTGTATCTCTCCTCCTAGCTGTGTATATCCAACCATACATATCCATTCTCACTGGCTTTCCTT
TTAGTCCCACTGTTGAGCCAGACATATTGAAGAGAGGAAAAAGGCATTGTGGGAGGTGAGGTATAAAAGTGCATCTGATTCGGCAGACTGCCAA
GTGCCTTCTTCTCGCTGAGAGGGCACTCAGTGACTACCTCTGCCAACAGGAATATAAACTATCCATTTACTGAATGAATATCTTCTCTGGGTCA
TGATTTCACATCAATGTATTTGGAAAATAAATGTTTAAGGAAATTCATTTTGTTTCATTATAAAACATTTTACTCCAAAATTGTATCTGAAGCC
AAATCTGGTCTCATCTTCTTCCATTGCCAGGGAAGATTTGTGTTAGCCAGATTTTCCATTATGATAGGGAAATATCTGAGATGTCCAATTTGTA
AAAAAGAAGAGGTTTGACTTAAGCCTTGGGGTATCCTTCTTCATGACTGATTAGCCCTATTGTGCTGACTCCGTGGCAGGAATGTTGTCTTGTG
GCTGTGAAGCAAAAGGGAAATATATGGGGGTCAGAGTTCCAGGACCCCCTTTAAGAATAAAGTCCCCCAAGTCTAAAATCTTCTCATTAGGCCC
TTCTTCCCTAAAGAGTTGAGAACCTTCCAACAACAGCATGTCCCTGGCTAGACACTAAGCTTCTGGGACATTGCGTTCAGCCACAATCATAGCA
CAGGTTGGACAATACCTGAAAACATGGGCCTTTGGGGACATCGAAGGTCCAGACAGTAGCAGAGTTCATGTAGTCCTACAAGCTAACCATACGA
GACCATAACTTTCTTGAGTCCCAGGTCTGCCCACTACTGAATTCTTAGTTCTGCTCATCTTTAGTATTACCTGTCTTTGTAAACAGTAAGTGAA
TGGTGCCCACCTCCAAGCCTACTCAAGTAGATCATCTCTGTTTCCTCCTTCCTGTTTCTGCTGAGCTAATTCACTACTGCTGACTAGGAAGACA
GGGTACATACAATTATGTTTAAGTATTCCTTCAACTTCACCATGCATTGGAACCCATCCACTTTTGAAATATTTATAAGAAATATATAGTTTAT
TATAAATAATATATAATCATGTTTTGAGAACATTTTTAGATTATTAAAATGATAAATGTAATATAGTATCTTAAATTTATTAAATATCATTAAT
TCATGGCTGTGTATTTTTATTAAATAATTTTGGGATAAATTAAACAGTATACATTTAGAATACTCTTTTAAAATATTTAAATTAAATATAATTG
TATGAACTTTATCATACTTAATGAAAGATGGGATTTACCCCTGTGAAATATGCCCTCTTTAATCTGACATGGATAATCTTTAGTTTCTTTTTCA
TTAATGGGTAAATAGGACTTCCTGAAGAACCTACCATAGTAAGTCCCTGGGAAACATGGCACCTTATCCCTCCTCAGCCCTCGCAGATGTGTCC
TGTCTGCTGTGACACTAGAACACCATTCAACCCCTGGGTGTAGATTTCACTTATGACCATCTACTTCCCGCAGGAGACAAGTGCCTGTCAGACA
TGCCCTATGACAGTGCCAACTATGAGAAGGAGGATATGATGACATCCCACGTGATGGACCAGGCCATCAACAATGCCATCAACTACCTGGGGGC
TGAGTCCCTGCGCCCATTGGTGCAGACACCCCCCGGTAGCTCCGAGGTGGTGCCAGTCATCAGCTCCATGTACCAGCTGCACAAGCCCCCCTCA
GATGGCCCCCCACGGTCCAACCATTCAGCACAGGACGCCGTGGATAACTTGCTGCTGCTGTCCAAGGCCAAGTCTGTGTCATCGGAGCGAGAGG
CCTCCCCGAGCAACAGCTGCCAAGACTCCACAGATACAGAGAGCAACGCGGAGGAACAGCGCAGCGGCCTTATCTACCTAACCAACCACATCAA
CCCGCATGCACGCAATGGGCTGGCTCTCAAGGAGGAGCAGCGCGCCTACGAGGTGCTGAGGGCGGCCTCAGAGAACTCGCAGGATGCCTTCCGT
GTGGTCAGCACGAGTGGCGACCACCTGAAGGTGTACAAGTGCGAACACTGCCGCGTGCTCTTCCTGGATCACGTCATCTATACCATTCACATGG
GCTGCCATGGCTTTCGGGATCCCTTTGAGTGTAACATGTGTGGTTATCACAGCCAGGACAGGTACGAGTTCTCATCCCATATCACGCGGGGGGA
GCATCGTTACCACCTGAGCTAAACCCAGCCAGGCCCCACTGAAGCACAAAGATAGCTGGTTATGCCTCCTTCCCGGCAGCTGGACCCACAGCGG
ACAATGTTGGGAGTGGATTTGCAGGCACCATTTGTTCTTTTATGTTGCTTGTTTGGCGTTTGATTTGCGTTGGAAGATAAGTTTTTAATGTTAG
TGACAGGATTCCATTGCATCAGGAACATTCACAACATCCATCCTTCTAGCCAGTTTTGTTCACTGGTAGCTGAGGTTTCCCGGATATGTGGCTT
CCTAACACTCTCCCCACCCACCCCACCCCCCAAAACAGAGCCTGAATCTTCATGAAGTGAATAAAACAATTATCCAAGAAGGAGTAAGGTGGAT
CTTGCCCTAAGCAGAGTTTATGCCACAAAGATTCTCCAAATCCCCCAAGACAGCACAGCCACTGGGGTTGAGCCATCTCAGGGAGCTCTGCAGG
TGAGCCAGACGACCAGATATAAGGCAGCTCCGGAGGAGCAGGGACATCAGCCTGTGCAGAGACCAACGCCAAACGTTGAACTTTGAAAGACTAT
TAAGTCATATATTGTATGGCAATATGCTGTCTGGACAAGTTGTGCAATGTGCTGAAGGGAAGGGATTGGAGAGCCTTGAAGACTCTTCTTCATT
TGCCTGATCAACCCGGCCTCCAGAGGGTTTTTTGCCCAGTAAGACGAGCTCAGTGCTCTTGTGATCATTTTTCTCTTATCGTTTCCATGCCGTT
GATGGCCCTGAACCTCATCACTGCATTTTAGAACCCAATCCTGAAATTGAGACCTTTTTTTAAACTTCTGATACTGTAAAACTTCTTGGAAGCC
AAAGCTTTCTTCCAAGCCCCATCCTCAGTTATCCTGGTTCCTGTTCTTCCCCGAGCTGATAGTACCAGGACCTGTTATTCCACAAAAGCACAGG
CATCCGTCACTTCAATTCAATCCCTGTTCAGATTATAGATATGGACTTTGCTATCTTGATAAATGTCTTCTCTATGTTATTTTGTCTGAAAAAC
CTATAAAACCATTATTAAGAATGACCATTTTTAGATGGAAGAAATGAGCCCAGCATCTCAGTGGCTAAAACACAAAATATCCATGCTTTTAAAC
AAAATTGTTAAATATTCCCAAGCTCTCTAGTATAAACACCAAGTAGCATGTGTTTTCACATAAAGAAGACAGGGGCCATGCAACCTTTATCAAG
TGGAGGTATTAGAATGTTGTAATGTTTGGAGACACAGTGTGACCAGTACAGGTTCCCAGAGAGGAATGCCCACCATATCACAGAAAGGTAGAGG
TGGGATCTGCTATAGCCAGACCAAGACACCGATGTCACGCTGAAGCCAAGTCAGTTAGCTGAAGATTCTCAACAGCAAGGCCTCTCTTAAGAGT
CAGTAATAGGGTTGTTACCATCCACCACCTCAACAAAACAAAAAGCTTATAATTGTAAATGTTTACAGCACTGTCTTCCCAGAAACTTTCTGAG
GTGATTCCAAAGAACTAGAGGGGAGATGGTCTATAACAGCTCTTGAAGTAAACGAGGTTCTTAGTCTCAGCTCTCCTGACATATAGGGCTTGAT
TATTACTGGTAGGGATTGTTCTGTGAATTGCTTACTACTACCCCTGGTCTCTCCCCAGTAGATGCCAGGAACATTCTAGCTGATACCTAACTGT
CTTCCCAGGTGTTCGAGGGAGCAAACCACTGATCTAAACTCTAAACGCTGAACTACGCAGGTTTTCTAAAAATGACAAGCCCTTGAAACCTTTC
CCAGTAGGCAGCCTCGAGCTGGACTTGTGTCTTTGGAATGCTGATGAATTCTATAGATCAGCATTGCAAATACACTTCAAATACGTCTGAGTTC
AAGTGCAGGGACTGAGTTCACCAAGGTGTCAACTGTGCTCAAAAAGTTCAAAAGTGTGTGTTTCTTTGTTTCTAAAACATTGTGGCATCTTTTT
CATTTGTTTCTAAAACTTTTTTTTTAGAAACAAATGAAGCACTTGGAAAGTGAAAGTAAAATTACAAATATAAGGATTTACACTGAAGAGAGAA
AAATTTTAGGAACTATACCTGTGAAAAGATTTTCTTCAAAAGGCAGGCTAGCCTTACCCAAATTCATATATGGCAGGTGTCAACCTCCCAAGCT
TACAGTTAGCAGGCAGCTTTTGCTCACTCATCCTTAGCCATGAGAGCCATTAAGTGTGGTCCAAGAAAGATGGCTCCAAACCCTACCCCCGACC
CACCAGTGGTATTCAGAGATTAAAGCAGAATTGTAAATAGTGGCTTCAGGAGCTCTTTTTTAGAATGCTTTGCCCCTTCCTCTCACTGCCTTTT
TTAGCCAATATAAATGTCAATTTGCACACCTTTTGTTGTGGTTTTATATTGTAACAGCATTTTTTTGAAACTATTGTATTTAAGATAAGGTTTC
ATATTATGTCCACAAGTAATTAAATTATGTTTGAAGGTGGCTATATGCTGTATCAGAAGTTGATGATGTTTTTCTTTAGCCGGTAAAGGAGGGT
TTTGCATGACCTCACTGTTTGTTCTGTGGTTTCTTCTGTTGTATGATGTCTCTCTTGAGTTTTGCTGTGTCATGAAGTGCGCTGAGATTCCAGT
GCCCTCAAGTTGTGTTTTAAGTAGCTATCAGAGGCAAGAGGCTTCCTAAGAGCAGGTTGACCTGTTGGCGACAGATGGCAATCACCATTTCTCA
TTCCTTCTTCTCCCTGTTACCCCAGCTTCCTGTCCCAGGTCCCTTCTGTGATTCTTACCTTAGTGTGCATGTGTGTCTGTCCTGGTGAGAGTCA
GGAGCATCGATATGTTATCATTGCATTATCACCAACGGCACGCACAGCCTAGCACCTGTTGCTTCACATACCGTCACACTCTGTTTCCAATTTA
GATACAACCACATAATAAAATGTTAGAGTCTTCAATGGGAAGCAGAGGTGCTTGTTATAAAGATGGCGGCTTATGCTTGTGTCACATTTTGTGT
TCTTTTCTTCTTTTGTTTGGTTTTAACTTAATTGTGACCCTTGTAACATCATCTTGCCAAAAAAAAAAAAAAAAGTTGAACTGGATTTATGTAG
ACATGTCAAGACGTACTATCTATTTCTTTGTCAGTTATAGCAATAAGAGTGGATAAACTCTAAAATCCAGATCTCCCACAATGAACATCCGTGT
TCTTTCTATGATTTTTCTTTCTTTATGGTGAGCCACAATTAAACTTGAGATGTACAGCCACCCAAACCCAGGAAGCTCATGTGCATCTGGTGCT
ATGGCACTCACTCTGAATAAGTGTGACCAGATATTAATATCCAATATTGTTTCCAATCCTTTCTAATACATTTTTTCATGATGTTGTGTGTGGT
TAAATCTTCCTGTATCACTACCACTTCAGATCTTCACCTCCTTGCTGACAGGTAAATGGAAGTGTGAGTAAGAAAATGAAGCCATCTCTGCAGC
CCTGACTCTAGCATGATATTAACTCATTTCTGTTTAGCTGCGAACAAGAGGTTTCCACTCGGAGCATAGAGGAGCTATCCTTTTGTACACATTT
TGCCTTAGATCACTACAGACGATCATTACAGAGCAAATAAATTCTACAAATATTCTGTCTGCCCCATCCAGTGAAGTCCAAACACAAGCATAAA
ACAAGCCCCAGTCATTGTGGGAGTCTGCTGTAGCCTCTCTGAAACCAGGAGGTGAGGGAGGAGCCCAGCCCTTGACACAAGAAGTGTGAGCAGA
TACACAGAAAGAGGCACAGCCAGTTAGCTCAGCTGGCCAGCTAGGGAGTCACTGCTAAGCCTCTGGACCTCAGCCACGTGGTGAAGTGGTGGCA
GAAGAGGAGAGCATTCAAGACAGAATGACAAACAGAATGACAGCTTTCGTGTCCTGCCTTTTATTCCTTTCCATTTTAAATAAGCACAAGATCT
CAGGATGATTTTCTGACACCGTGGAGCAAGGCTTTCTCATTCATTTATCTGCTTCACTCTGGAACAGATTGCCAGAGAAAATGATGCTAACTTC
ACTTCTAAGGAGTATGTCGATGTAAACACTGGGCTCTGTTTTCCATGTGAGCCTGGAATGCGCACTGAGGGATAGGAGGGAGGCCCAAGGTCCC
TGACCCCATTTCCAGTGTTTCTCAGTCATATTTTGATCAGCAGAATTCAGATTTCTACAAGCAGAAAATGATGTCCAAGTTCTGTGACTCCTTA
GTTAAACTTCCTTGCCTTCCTTCTTTGAACCCATTCCAATTCTCAAGAAACAAACCTTTGGGCACTAGGGATATGGCTCAGCCGGTACAGTGCT
CATCAATGCCCAGTGCCCTGGGGTTAGATCCCCACACACTGGCTGTGGTGGCAGTACTTGTAATCCCCGTATTCTGAGGAAGAGGCGGGAGTTG
AAGGTCTTTAGCTCCACAGGGAGCTCAAGGGAAGCCTTGGATTCCTGACACCCTGTTTCATAAAAAGAAAGAAACAAAACGTTGGCTGTTTCTG
AAAGTTGCCTTTTCTCATGGCTTCAACTAAGACACAGAGCTAGGAGCAAAAGAGATAGAAAATTGAGCAATTAGTGAAAGGCTCAGAGGAAGTT
CTCAGATGACCAAAGTGGGAACTCCGTGTTCCTCCATCATTCTGGAAACTTCTGGCTCTTTCTCTGCACCAGTACCCAAAGCAGCAACCAACTG
GCACAGCCCCCATATGTAGAGGTGAGGTCAAATGTTGCTATTGCCACACAGCATCCACCTCTTGTGTCTTTTCCCAGGCTACTCTCTCCACCCG
TCACATATACAACCTCTTGGGAAACCCAAAGGGATTCCCATGGCCCCTAGAATTACACTGCTTTTAGCAGCCCCAAACCCTTTGGCTTTTCTAT
TCCACTATTAACCTCCACCCTGAGCAAACCCACTCTAGGATTAGCACACAAGAAGCCCAGCCTCCAGTTCATCAACTACAACCCTGCTGGGTAT
CAGTTGCTCTAGAACTGCTATCTTAGAAGCCTCTCAAACTCTACCCTATCTTACCCCTATTGTTCAGTTGCAGCTTCCAGAGGTCACTTACTGA
ATATTCTGGAGTGGAAAGTATTCATGGGACAACCCGCGGCCCCAGCCCTGAGGTGTGTGCGTGGCCAGCCCTCCAGTACCTGAAGCAAATCATC
AGTCAGTTCACTGAGGACCAGTTAGATCCGGGACTCTCCTGAGCCTGGGCTCACAGTTGTCTGCATAGAGGAACAGTGTCTTCCCACAAATGGT
CTGAAACAGCTTAGATCTAGACAAAGGAATCAGGTGAGAAGCTGAGATGTTCATTTTGCTACTCTCAAAAGGCCAGCTTCCACGTGTAACCACC
CAGCAAAAAGCAACCACATTGAAAGATGCACATAGTCTAACCCTCAGTACCTCAGCATTTCCCCCAGTGGGCAGCTTTGACATCCAACCTGGCT
TGTGTACACTACAAAGACAGAAGGCCACATGCCAACCATCGTGGGTTTCCCTAGCTACTCCATCATAGATGATCTGATTTTCTGTGACTGTCCT
TGCAGGCCTCCTGCCTCCTGAGTCCCGCTACTGTGGTCCAACCATTCTGGGCTTTCAAAAGTGATTTGTGGAAATGGGGACCTGATCCCACATT
ACTGTCCCTACGCACAGGTCATCAGTGGTTGCACTGAAGTTTCAGAGCAGAGCAGAGTTTCCTCACCTGCGAACATGGACGTTGAGGTTGCATT
GCCTTAAGCAGGTGTCCCCCTGGGTGTGCTCCCAAAAGTATGGTCTGGTGCTTAAGAACAGAATTCTGGAGACCACCAAGTCTGTGTTTGAATG
TCTGCATTGCAGACTAAAGGAATGCAATGGTGGGCAGGTGACCTACCCTTTCCAACACCTCTTTCCTCATCATTGACAGCTGGAGAGCTGGAGG
CTAAAGCTAGTAACATCTGGCAAAACGTGAGCACTGGGCCTGGGTCATTCAAATATTCTGTTCTTTCTGACTTTTTTATATACTATCCTCTGCC
TCCTATCTTATCTCTGTGACAACCGGACAAGCCAGCAGTTCATTCGTATTCCAACCTTGGAGACCAGGTAGCTGTAGTGGGGAAGCAGCAAGCC
TCTCCTCCCTTGTAGCACCAGCCACACTGAAAAGTCCCCAGCCTTTGCAATTTCCCTGGGAGTTGCAGGGTCATGCCCCATTGGACACATGAAA
GACAGCACCTGGGAGGCCAAGCGTCACAAAAAAGATGCTGCTGTGCAGAGGTAGCTATAGCTCCAGCCCCTGCCCCAGGCCCCTTCTCCACTTG
GCTCCCGCTCCATTCTGGAATGTTCCTTGCAAAGAAAAGTGAATATAATATGATCCTCTTGAGATACAGTGTCTCTCTCAGAAAGGGAGGAAAA
TACCTACACCTATAAACTTGCCTTCAAACCACCCCTCAGCTGATCATCACCATACTGGTCCACAGGTTGCATGTCATATCAGTTTCTCACAAAG
AGTCACTATCCCCAAGACTTGCCCCAGAGCAACAAGGCAAGTGGCACAGGGCCAGAATCCAGAGCCAAATAGGAAAAAATAGTGACATGGGTAG
AGCCAGGCACAGGGAGCCAGATCTTGTTTAAATATGACAACTGTGGACATCTCTAGAGCCCAGTGCATGGTAGTTGGACGTGGGCAGCATTGTC
CACCCAAGATGTAATCGAAGCTCTGAGCTGTACTGCAGCTGCTCTGGAGGGTAGAAAACACTGCTCTGGGAGAGGGGAGAGGAACAGGAAGAGC
TGGCAAAGGAAATGAAAATGTTTGTACATGAAGGAAGGAAAGCCAGTTGAGCAGGCATCCCCAATTTTCATCTTCAAAATAAGAGACCAATTAC
AAATCCAAAAAAAAAGAAAGGCCAAATGAGGACAGCCACTGAGGTGGCCTCTGAGCCCAATATATAGTTTGCTCCAAATGACCCTGAAGGTCAA
TCATGAGTAGCAAGAGGTCATACAGCAGAGGTAGGCTCTGACCCCAGGCCTAGCTAACAAGAGAAGACTTCAAAAACACAGTGCCAAAGTTCAG
CTGCTCTCAGTTTCTTGGCCAGATTTTACAAAACAAGGAAGAAGACCCTCACTGGTATGTCTCTTGGCTCTTTCCACGTTTTGACAAGGCTAAG
AGCAGGAGCAAAGTGGACATCCTTTGACCTCTGACTCCTCCGATGACTACAGACGCAGCATGTCTAGGGAGAGGGCTACTTATTGCAGGATGCC
CTAACCTCCAGCCCAAAGAACAGTCTGGTGGCTCCTTGTACAGCAATGAGCCCCTGACCCGTACTCCATTTACAGTAAGCACTTGACCTGCGGC
AGAGCAGCTGAGCCCTGCAGACTCAGTGAATCGGCACCAAGGGAAGCAGATGTGCTCGCCATCCTCCTCACGCACAACCAGGAACCAGCCACTA
GGGGAGGAAATTTGCATAATAGGGATAATTTGGTACCAAAACTGTCCCCTGAAGTGAACTACATGGATCTCGGTATAGTTCAACAAATTTCACT
TCTTCTTTCCCATGTTTTCTCACACCAACTAGATATGAGGTAGCATGTGCAACTCGTCCAAAGGTTCGGGGGTGTTGTTTGATTTTTATTATTG
TCTCCCTTCATAAGTCAGTCCCATGCCTTTTCTCTCTTCAACCCACCACTCCCTTGTGCCCAAAGTGAGTTTCTGAGCTCCCTCAGAGACCTCT
CCCAAGCACATGCAATGTGGGGGACACCCAAATGCTCTTACCCTGCAACACAAGCAGATTCACAAACAGCAGTGGTGGTCATCTGCCTCTCTAC
TGATATACTTGACCAATGCTGCCTCACTCTGCCGCTTCCTAGAGCCTGCATGAGCTTCCTCATTGACATTATCCCTGTGGCTTTCTGTGGGCAG
AGGGTGGACCACTCCCAGGGTACAAAGGGTGTGTGGTTTCAGGGCTCCCCAGTTTCATTCCCCTTTCACGCCTCCTCCTTTCGTGGTACCACCC
TGATTCCCACCCACAGAGGCACAGCCCTTCTCTAACCCTTCGAGGGACTCCCTATTGCCCCCATAAATTCCCACTTCTGCAAGCATCAGGAGCT
ATTTTTACCATTAGTTTTATAACACCTCACAGGAAGGCTGTTGTGCATTTATATCTACTGATACTGTTACCAAATTTTACGTGACAAATACTCT
AAAATAAACCAAAGGAAAGAATAGCCTGAGAAAAAATCCCTAAGACACCACCAAGATGGCCAGATTGCTGTTAAAACACACACACACACACACA
CACACACACACACACACACACACACACACACACACACACACACCCTAAGTAGCCAGTGTGTTTTATATCAAAATTTGGGAGAAAAGAATTATAA
ACCTACACTCTCTACAGATAACACCGACAGAACAATGTGAGGTAGGCATACCCTGCCACAGGAGGTTAGGCCTAGGGCAGGCTTCCTCTTCTTC
CTGGTTGGCTGGTCCCACACGCAGAGTATGAAATGGCCTTGATGGCTGATCAGCACACACAGATGCTTAGATTCTAAACTGAAGCCTTTCGTTT
GTTGCAGCTGCTGACTTGACTTGTGGATTCAAATGAGTTAAAATAGGCTTCTTGTCTTTCCTCACTCCATTTTTTAAACATTTAATTTAATTCA
GTATATCTCAAAATACAAAAGTAAAGAGGACACTTTCTTATGAGTTCAACCTCACGGCCTCGGGGACCATAAAGTGCAGGAATCTATGGGTAAA
AAGCATTCTCCCCATCGTGACAGCCCTGTTCCCAACCCATCTGTTCAGACATCTTCCAAATCACATCTAATTCCCACACCATCCTACTCCAAGA
ACCCTGCTTACCCTCAAAGTCTTCCCACACTGCACTGGCTGCTAAAACAGAATTCTCGGCACTCGGTGTTTGGTGCTTCTTCGAGACCAGATCT
GTTCCTACACTAACTTTGCACTTGATCATTCCTGGTCCTGGCTGTTAAGTGGTAGTCTGTCCACATCAGACTAAAAGCTTAGAGGGTTCCAGGC
TCCCCAGAGCCCCCCATAATAATGGTATCCTGTGAGATCAGTCCTCCCTGTATCCTGGGCTCCCTGGCTCTCAGACGGGTCCTGTCCAAGGTGA
TGATCTGGACCCTAGCCCTTCTTTGAGGAGCTCCTTCCCATCTTTGCACTGCTTCAGCTTTCCCCTTGCGAGTGGAAAATGCAGCAGCTCTCAC
CTTGGAATCTCCAGGAGGGGCCACTCCTTGCCCCAGTATTCAGGACAAAGTATCCTGGCACAGGCAGTGGCCTTGGTGTGAAAAAAGCAAAGAG
AGGGGTCATGAGGCAGAGAAGGTGAGTGCAGCCCCTTGAAAACAGGGAAACTAGCCATTTTAGCCGCAAGTACAGGGCAGAGGGTGTCATTCAC
AGATTGGGACTTTTCTAACTTAAGTAATTGTTATTTCTATCAGAATATACTGGTATAAGCCAGTCCTATAAGCATGGTCAAGTTCCATCCCACT
GACTCTAATCAGGCCAGAAGTTGACCTGTCAGGTCCCAAGGCCAGTAATTGAAATATGACAGGACAACAGTTCACTGTTAAGGGGACACCAAAG
TTTTGCCAATAACTACAAGGTTGTTAGATGTTCAGAGTCAAGCACTGAACAGACAGGACCTAGAAACCAGTCAGGATCCACAGACCAGGGGCAT
CTCAACCAACTCTTGAAAAACAGACAGCAGAAAGAATGAATAGTGGAGACATGGAAGAGAATTAAAACCAGAGATGCCAAGTAGAGGAGGAAAC
AAAAAGAAACAAAGAAGCAGGGCGATAAATTTATTTAAAAACAGTGACATACGTAGTAGGTGCTAATCAATCTTTATCAGCAATCATGCTAAGT
GCCAGTGAACAACACACACCAATTAAAAGGCAGACACTGGCAGAATGGACCCCAGCTAAGACTTGTCTACAGGCTGTAGGCTGCTAGGTGAGAA
AGGTTACTTATGATCTTACCCAGCTGTGGGCCCTGTGTAGTACAAAACCAACCTACCGCGCAAGATGTGCCCGCTATCATAACATAATAGTTAT
GATAATAACCAACCACTTTCTGATTGGATTTTAAGCCTGCTACACTAGAGGGAAATCATACGTACTCAAAAGCCCATGATGAGGGCCTTCACAG
GCCCTGGTGAGAGCAAGGGAAGAACTTAGCTTTTACTTAAGGATGTGTTTATATCCACAGACTAGGTTTATAGCTATAAAGCAACAC
MOUSE SEQUENCE - mRNA
CCTTGGGGAGGCACAACTCTGTTGATAACCTGAAGACAATGGATGTCGATGAGGGTCAAGACATGTCCCAAGTTTCAGGAAAGCAGAGCCCCCC
AGTCAGTGACACTCCAGATGAAGGGGATGAGCCCATGCCTGTCCCTGAGGACCTGTCCACTACCTCTGGAGCACAGCAGAACTCCAAGAGTGAT
CGAGGCATGGGTGAACGGCCTTTCCAGTGCAACCAGTGTGGGGCCTCCTTTACCCAGAAAGGCAACCTCCTGCGGCACATCAAGCTGCACTCGG
GTGAGAAGCCCTTCAAATGCCATCTTTGCAACTATGCCTGCCGCCGGAGGGACGCCCTCACCGGCCACCTGAGGACGCACTCCGTTGGTAAGCC
TCACAAATGTGGATATTGTGGCCGGAGCTATAAACAGCGAAGCTCTTTAGAGGAGCATAAAGAGCGATGCCACAACTACTTGGAAAGCATGGGC
CTTCCGGGCGTGTGCCCAGTCATTAAGGAAGAAACTAACCACAACGAGATGGCAGAAGACCTGTGCAAGATAGGAGCAGAGAGGTCCCTTGTCC
TGGACAGGCTGGCAAGCAATGTCGCCAAACGTAAGAGCTCTATGCCTCAGAAATTTCTTGGAGACAAGTGCCTGTCAGACATGCCCTATGACAG
TGCCAACTATGAGAAGGAGGATATGATGACATCCCACGTGATGGACCAGGCCATCAACAATGCCATCAACTACCTGGGGGCTGAGTCCCTGCGC
CCATTGGTGCAGACACCCCCCGGTAGCTCCGAGGTGGTGCCAGTCATCAGCTCCATGTACCAGCTGCACAAGCCCCCCTCAGATGGCCCCCCAC
GGTCCAACCATTCAGCACAGGACGCCGTGGATAACTTGCTGCTGCTGTCCAAGGCCAAGTCTGTGTCATCGGAGCGAGAGGCCTCCCCGAGCAA
CAGCTGCCAAGACTCCACAGATACAGAGAGCAACGCGGAGGAACAGCGCAGCGGCCTTATCTACCTAACCAACCACATCAACCCGCATGCACGC
AATGGGCTGGCTCTCAAGGAGGAGCAGCGCGCCTACGAGGTGCTGAGGGCGGCCTCAGAGAACTCGCAGGATGCCTTCCGTGTGGTCAGCACGA
GTGGCGAGCAGCTGAAGGTGTACAAGTGCGAACACTGCCGCGTGCTCTTCCTGGATCACGTCATGTATACCATTCACATGGGCTGCCATGGCTG
CCATGGCTTTCGGGATCCCTTTGAGTGTAACATGTGTGGTTATCACAGCCAGGACAGGTACGAGTTCTCATCCCATATCACGCGGGGGGAGCAT
CGTTACCACCTGAGCTAAACCCAGCCAGGCCCCACTGAAGCACAAAGATAGCTGGTTATGCCTCCTTCCCGGCAGCTGGACCCACAGCGGACAA
TGTGGGAGTGGATTTGCAGGCAGCATTTGTTCTTTTATGTTGGTTGTTTGGCGTTTGATTTGCGTTGGAAGATAAGTTTTTAATGTTAGTGACA
GGATTGCATTGCATCAGGAACATTCACAACATCCATCCTTCTAGCC
MOUSE SEQUENCE - CODING
ATGGATGTCGATGAGGGTCAAGACATGTCCCAAGTTTCAGGAAAGGAGAGCCCCCCAGTCAGTGACACTCCAGATGAAGGGGATGAGCCCATGC
CTGTCCCTGAGGACCTGTCCACTACCTCTGGAGCACAGCAGAACTCCAAGAGTGATCGAGGCATGGGTGAACGGCCTTTCCAGTGCAACCAGTG
TGGGGCCTCCTTTACCCAGAAAGGCAACCTCCTGCGGCACATCAAGCTGCACTCGGGTGAGAAGCCCTTCAAATGCCATCTTTGCAACTATGCC
TGCCGCCGGAGGGACGCCCTCACCGGCCACCTGAGCACGCACTCCGTTGGTAAGCCTCACAAATGTGGATATTGTGGCCGGAGCTATAAACAGC
GAAGCTCTTTAGAGGAGCATAAAGAGCGATGCCACAACTACTTGGAAAGCATGGGCCTTCCGGGCGTGTGCCCAGTCATTAAGGAAGAAACTAA
CCACAACGAGATGGCAGAAGACCTGTGCAAGATAGGAGCAGAGAGGTCCCTTGTCCTGGACAGGCTGGCAAGCAATGTCGCCAAACGTAAGAGC
TCTATGCCTCAGAAATTTCTTGGAGACAAGTGCCTGTCAGACATGCCCTATGACAGTGCCAACTATGAGAAGGAGGATATGATGACATCCCACG
TGATGGACCAGGCCATCAACAATGCCATCAACTACCTGGGGGCTGAGTCCCTGCGCCCATTGGTGCAGACACCCCCCGGTAGCTCCGAGGTGGT
GCCAGTCATCAGCTCCATGTACCAGCTGCACAAGCCCCCCTCAGATGGCCCCCCACGGTCCAACCATTCAGCACAGGACGCCGTGGATAACTTG
CTGCTGCTGTCCAAGGCCAAGTCTGTGTCATCGGAGCGAGAGGCCTCCCCGAGCAACAGCTGCCAAGACTCCACAGATACAGAGAGCAACGCGG
AGGAACAGCGCAGCGGCCTTATCTACCTAACCAACCACATCAACCCGCATGCACGCAATGGGCTGGCTCTCAAGGAGGAGCAGCGCGCCTACGA
GGTGCTGAGGGCGGCCTCAGAGAACTCGCAGGATGCCTTCCGTGTGGTCAGCACGAGTGGCGAGCAGCTGAAGGTGTACAAGTGCGAACACTGC
CGCGTGCTCTTCCTGGATCACGTCATGTATACCATTCACATGGGCTGCCATGGCTGCCATGGCTTTCGGGATCCCTTTGAGTGTAACATGTGTG
GTTATCACAGCCAGGACAGGTACGAGTTCTCATCCCATATCACGCGGGGGGAGCATCGTTACCACCTGAGCTAA
HUMAN SEQUENCE - GENOMIC
AAAGTAGCATACAGGTTCTGACCTTCAAAGGATCAATAAACAAATGCATCTCCTGCTCTACACTTTAGCCATTGGAGCCAAATCCCATGGTTCT
ACCCTCAGTTTAGTCCCTCGGCTTAATCATCTTTTGATTGGCCATGGAATGTGTACCTAACTATAGTCAACAGTCTTACAAACATATATACATC
CTGTATATCTATTTGTACAAAGAATTGGATCTTCATCCTGCATATTCTCTTTTCAAAATTAGCCTTTGTTTACCATCTATAAATCAAGACAACA
TTATTCCTTCTCCCAAAATGCAGGGTTATTCAATACAACTTGTTGGAAACAAACCGGCTTGGCTGAAAATGTTTATGAGCTATAATCATAGAAA
ATAAAGGGATATTATAATGAGAAGTCAATCTTTCAGTTTAATTGCAGAACACTTTTTAAGCTACAGAAAATTGTGAATTTGAGCTCTGAAGTGG
AAATAAAGTTGCCAGTTTCAGACTGCATATCACAGAAAATGAATTCTTTTTCTTCATTAGAAAAACATATCTGCAAGTAATAAGATATTATGAG
TAAATATTACAAGAATACATTGTACTGGGCTGTGTGTAGCTACAGAATGCATTATATCTTTAAATTATTTCTAAATCACAAATTTAGAGAAAGT
CCTGCTCCTGGCATTACTAAATTATAATCTGTCTTCTTATCCTACAGAAATAAAGAGGTATCAACTCACTTAGGATAATTGCCTGATACAACCA
GAACAGGATTAGGGAAAATTCATTATGGTATTGCAGTGTTGTATTTTAAAAGTATTCTCTGCTTCCTTTTTGAAAAATGGCTTAAAAATGAGCA
GAATAATGCACATACAAGGAAAGTGTGAAATAAATTGTCAACTTTTAGTTGAAGGAAGGAAATTTGGAATATTCTGTGTTGAAGCCTTTACTGC
TAAGACAGCATTAAATAGGTGAAGAAGCAACACTTCTAGTGCATGATCGCAGAGCCCCAGACAGGACAGAAGCATAGGGAGGGATGGGGGTCTT
TCAGCTGTCAGGAGAGGAAAACACAGTCCCTCTGCATATTGGAAAACAAATGTGCCAACTCCTTGACATGTTACTTAAACAAAAATATTTTTCC
TGTAAGTAAGATAAACCCTTCAAAAGTAGCACCATGAAATGGAACTATCTGCTGAATATCTGCTCACTGTTCTAGGAACTGTGAGGAAACAAAA
TAGAAAAAAAACCAAATCTCTCCTTTCAATGAGAGCAAACAACAAATGTTACCAATGAAAACCAGAGAGAAGGCAGGAGAAAAGCTAACGCTCG
GGTGGGTCAAGGTCGTTCCCTTTCATGGAGAACAGACAATGACAACTCAACCACAATGATAATGGACCAAACACCTGAAGCCAGTAAGGGGGGT
GGGCAGGCAGAAATCCAGGGGAGAACCTTCTAGGAGACAGCAGTAAGTGAAAGGCAGTGAGCTGGTCTGGAGCACTTGAGGACTATAAGGTAGG
GCCAAGTGGCCAGAGACAGGCAAGTAAGTGGGAGAGTGGAGTCTGGCCTGCATTCAACACAGAAGCACCCCGATCTGACTCTGCTCTTCATCAA
GCTCTCTCTGGCTTCTGTGAGGGGCAGGGGTGGAAGTAGAGAGACCCCTGAGGAGGACACTGCTATTTGCAGATGAGAGGTGACCAGCCTGTCT
GCAATGGAGGGAGAGGAGGTCCGACCATGGACACCTGATACCTGCAGAGCTAGTGGGATTCCCCAATGGACTGGATGGTAAGTGAGAGAGAGAG
GCATCAAGAATGACTCCAAGGTTTTGGCCTCAGCAATTGCCAGCTGGTATTCAATGAGATGAAGGGGTCTCAGTGAAGCAAGTAAGAGAGGAAG
ATCAGGGATTCAGCTGGGAGACAGTAAATATAAGTTGCCTATTAAGCCTCCAAGTAAAGGTGTTAAGGAGGTGGGAGAGTTCAAAAGGAGGTCT
GAAGAGCAGATATTAATTTTTGGAGTAAGGGTACAGAGATGACATTGCAAACTGCTTCTGGAGAGGCCTTCAAGGAACTGACTGCGGAGGGAGA
CATGTCTGGAGAGCTGCAGCATTTTAGGAAAGAGGGGAGAAAACAGCCAATGAATAGCACAAGAGAAAAGCATGGCGAGTGGGGTGTCCTGAAG
ATCTACCTCACTGATATCGGGGTTCAATAAAATGTTCGTGCATTTTTTGGTAAATTTTAAAGTGCTAGATAAATGCTGACTTACTACCTTTGGG
ACCAGATGGACTTTGCTCCATCCATTGAGATCATTCTTTCCAGCTCTTCTTAGATTAAAAAAAAAATCCACTTTTAACTTAGTCAAGGTTAGTC
CCAACTGAGAATCTGGAGACACATTCCTGGGAATCAGAAGCTCTGCCTACTTTTGACTTGTTAGATAGCACTTTCTGAGAAATCTTACAGCCAC
CCATAAACAGGGGAAAGGGAGAAGGGCTAACTCACATTGATTACTAGGTATTACAATTGAAGACTTATGTTCATGCTTTTGTTTAATCCAAACC
ACAACTCCATGAAGCAGGATTCATTTTCCCTGTTGCAGAAATGACAGTGTGATCCTGAGAGAGGTGACGTAACTCAGCTGAGTTCACGCAGTGC
CCTCGCCATGCACCTGTGGTCCCAGTTACTTGGGAAGCTAAGGTGGAAGGATCCCTTGAGCCCAGGAGGTGGAGGCTGCAATGAGCCAAGATCG
TGCCACTGCACTCCAGCCTGAGTGACGGAGTGAGACCCTGTCTCAAAAAAATAAATAAATAAAAATAAAGAAAAGAAAAGAGAGTTGCTATCAT
AATTCAAAAATAACTAGACTAAATTTCAGTCCTACTTGATAGGCTGGAAAGGCTGTTCACATATCTTACTCTTTCCTGGACAGGTCCATTCCAT
CTGACCTCTCTACCAAGACTGTTGCTTCTAAGACTGACAATTCGGCCTTAGGGGAAATAAAAAATTTAAAGAGAAGTGAAGGAAAACGCTTCCC
ACAAATTGGAGAAAGGTGTCTTAAAACAGCAGTCCCCAACCTTTTTAGTGCCAGGAACTGGTTTCATTGAAGATAATTTTTCCACAGAGGTCAG
GAGGGGATGGTTTGGGGATGAGTCAAGAGCATTACATTTATTGTGCACTTGAGCTCTATTATTACTATTACATTGTAACATATAATGAAATAAC
AACTCACCATAATGTAGAATCAGTGGGAGCCCTGAGCTTGTTTTCCTGTAACTAGATGGTCCCATCTGGGGGTGATGGGAGACAGTGACAGATC
ATCAGCCATTAGATTCTCATAAGGATCATGCAACCTAGATCCCTCACATGTACAGTTCACAGCAGGATTTGCAATCCTAAGAGAATCTAATGCC
ACCATTGATCTGATGGGAGGTGGGGCTCAGGAGGTAATGTGAGTGATGGGGAGTGGCTATAAATACAGATGACACTTTGCTTGCTCACCCTCCC
TATACTGGTCAGTGGCCCTGAGGTTGGCAATTCCCGTCTTACAGAAATCAGTTCTTGGAGACATCTCATTTCTGTCTTGGGAAATGTGGCATAA
GCAGGAAGAAAGAGGATACGTGTGGAGCCAGAGGAGGAGCCACCATGATTCGCAAGGTAATTTGAAAAGAGAGGCCAGACGCATGGCTGAAATA
TTATAACTGGCCAAGATTTGTATCCCAGCCTGGTCACATAAAAACTGAATGCTTCAAAGTGCCGAACACTTACCCAGTGCTTGATATCTGCCAA
GGAGCCATCAAAATGTCCTTAATGTGCACTAACTCATTGAGACCTCAAACATTTCCGAGGATGTTTGTGCTATGGCAATGTTCCCATTTTACAG
AAGAGGAAACTGAGGCATGGACAAGTCTAGTAACTTGCAGAACTAACTCCAATAGTTTGGCCCCAGAGTCTCTCATTTCACAGCCACCTTCTGG
GCTTTCCATCAACCCCCAGCGAGTCCCCTTCCCTGAGCTTCAGTGTACTCATGTGAAATGGGGACCACAGTGCTAGCTTTGCTGCATCGTTAGA
TGGGAGATAATGCCTGCATGCCTGTCACAGAGCTCCCCTCAGGGTGGGGTGATAATTACAATATTACTATTAATCTTCAGTTAATTCTCTCTTT
TGAGACGGATGTTTCTAAGCTCCCCTGCCAGCCAAGTGTGGAGCAGAAAAGCATTTGCTCAGACCTCCTGTGTGATCTCTCCTCACCACCTCCC
TCCTTTTGTCCATCAAGCCTCCCCCAGTTCTGGCAACTCTTTAGTGCTATCCTTTGTGTTCCATAAGGCTCATGTCAGAATTTATTATGACACA
TTTTGCATCAGAATGAAACTATTAATTGTCCACCGACTTTACTACATTGTGAGCTCCTTAAGGGACAGATCTTCCTTTCTGCTTTCTTGTGACT
ACCTGCATCTCAAGAGCCACCTGCAATGGTACACTCTCAATAAATACAGGCTTTCTGAATAAATAAATGAATACATGCCACCAGAGTGTTTATG
CTTCCGGAAATCCTGCTCCAATCCTGTTCCTATTCAAAGAATCAACTGGAGTGACACACAGCTCTGTTCTTCTTTGTTCTGATCTCTTCAGGTC
AGATTAATATATTATTTTTTATTACTCATCAAAAACATCTTCAGAAACTTTTCATCAGAAAGATTTTTTGTGTAAACCTATCTATCTCCTCCAA
ATCACTATATCTGACTCAGTGTGATTTTTAGTCTCTTATAACCAAATGATATTGATAGAAATTTGTTGCTTTTGAGAACCTAAGTGTATTTCTC
TTACAAATTTCAAAGGGAATAAAGTGAAAGTATTATCAGGAAGTGGGAANNNNNNNNNNNNNNNNNNNNTGGGAAGAATTCATATGTCAGCTGC
AGCCCATCCCTGATTCGAAACCTACAGGCAGAGATGTGAATGGTCAGTAGTGCTAAAGGATTTCTGTGGTTCCTGCAGAACCAGGAGACACTGA
GGTGCCAAGACAGGGCCTTACAGATGTTTCAGGAAACCAACCACCCAGAGGAGTATCTTGCCAGGTTCACCAGGTGCTTCCTACCCAGGCACTG
CCAGCAGGACAGTCCTAGCTCTCCACCAGCCCCCTGCCTCTCCTCTGCATCCTGCTCCTTCACCCCCAGCAGCAGGCGGGAGCTAGTGGACTAT
GCTTAGAAATAGCCAAACCAACGAGGGAGCCCAAGAAAAAGGATCGAGGACCTCTCACCAGGCCAGTGATCATTACTTTTCAAAAACAGCAGAG
GCAGAAGCTGGAAGTCATGAAAACTGGGAAAAAGAAACCAAAGAAACAGTGTCTTTGTTGCAACAGTTTCATCCAAGTCCTGGGTATATAAATA
AAATAAAATTTTATTTTTTAGCAAAAAAAACCTTTTGCTGTTATTTAAAAATATTCTTGCTGTTTTCCAACCATCACAGGAGACATGCACTTTT
CATTGCTGTTAACAGATCTTTTTTTATTGAAGAAAAATACAATTATGATAACTTCCATAGATTATTTCATTGAATTTCACGTAATTCTATCAAG
ACCATTGTCAAGTAGATGTGCTCTGCGGATGAGAAACTGAAGCTCAGGCTTTAAACAACTTGCCAAACACAGTTACTAAGAAACAAAACTATTA
ACCATATCGTATCACCTCAATCATCCTTTTACTTTCCTTTGAAAGTAAAAAACTCACTCTCTTTTGAAAATCCCATCCAGGCTCTACCTGGACA
AAAGTGCCTAGCTGCCTCTCTTTCCATAGTGAGCTCTGAATAAGTAGCCTTTGATTAATCCTATAGGTGGTCTCTATTCTTTCGATAATACAGC
CTCATAAGAAGAAAAATTGGTGATTTAGCAATTATTGCCTGAATAAATTAAAGTGTTTTATTTAATACTTTAACATTATGAAGAGCGCAATATA
TGAATTAATATGTAAATAAAATAAAATGACTTTAGACATAATATGAAAAAAAACAGTTTTACATGTTCCAATTTCTCCCTATATGTTTTCCCAT
CCATTGTGCATTTTACTGTTGTCCCTAAGGGTTTGAACATTTGATTTATATCACCTCTCTGGTCCCTCTGAGTCTATCCACAGACCCCAGGTTA
TAATAATATAAGCTTGACTTCCTTCAACTCCACCCGGTTGGGGGCAAGGGTAAGGCATGGTACCTGCAGCCTGACTGCTGTTTTGTTTTGTTTT
GTGACAGGGTCTTGCTGTGTCCCCCAAGCTGGAGTGCAGTGGTGTCAATCATGGCTCACTGCAGCCTCAAACTTCTGGGCTCAAGGGATCCTCC
TGCCTCAGCCTCCTAAGTAGGTTGTTGGGACTGCAGGTGCATACCATAACACCTAATTTTTAAATTTTTAATACAAATGGGGTTTGATTTTTAA
AGTTTAAAATTTTTTTGTACAAACAGGGTCTTGCTTTGTTGCCCAGGCTCGTCTGGAACTCTTGGCTTGAAGTGATCTTCCCACCTTGGGCTCC
AAACTCAGGTGTGAGTCACTCCACCCAGCCAAGGAGCCTGACTCTTCACAGTGCACTCCTCATCTCATTGGCACATTGGTTGGGCTACCAGGGA
ATAACTGTTCTTACTTGGAAAGTACTCCTCTCTGTCAGGCATGTGGCAAGGTCTTCCATATGTGATCTCCTTAACCCACAAACAACCCCACCAG
GAGGGTTATATTATCACCACCAATTTACAGATGGGGAAATTCAGGCTTTGACAGATTAAGGACCTCAGTCAGATTCATAAGCCCAAACTATCTG
GGCTCTTAGCCCCACATGCTCCTGCCTCAGTGGGCTACACCTTGGTGAGATGCCTATGTTTCGGTCCACTGGTCCACTCCTGAAGACCAAGGCA
CAGGTTTAGACAGACGTACCAGCGGCTAAAGGTACTTCTGCATCCAAGAACTAGAGGTGTCCCCTGTCCCCTTTACAACATTGTTGGCCTCCCT
CTGGGCCTTGTACCTTATGTTTATTGCCTCATTTAATCCAGGTAAGGAGTCTTTAGGATTGGTGGCATGTATTCTTTTTATAGACAAGGAAGAT
GATGCTCAGGAGTTTGGATAACCTGCCCAAGGTCACGCAGTGGAGGAGAGGACTAGAAGGTAAATCAATGGACCTTTCACTACCTTCCCCTTCT
CTGGGCTGTAGCAGTGAGTTAACTGGCTCAGACTTGCCCAGCGGAATGCCCTTCAGGACACACTGAGTACTGTAGTAAGAGCTGCATATGCGCT
CAGGGAGAATGTGGATAGACGCTGTTTGTGATTCACGGGTAAAGGCCCTTCTCCCAGAGGCAGCAGAGGCACATCCTCTCTCTGGTGTGCCCTG
GCCCCCGACTTGGAGGCCTCCTGGGCCAGGCCAAGACCTTCCCCGGCAGCGATGGTCTCCAGCCACACTCAACTGCCCTGAAGGGACATTTCCT
GCTTATTCCCTTGCCCGGCTGTGTCCTCCACCCGGAAGGCCTGTGCCTTCTTCGCCTGCATGTCCTACCCTGAGGAGGCTCCCTTGGTCTTTCA
TCGCTCTCCCTATCGGTCTTCACGCCTTCCCGAACCACCGCGCCCAAGCACCACCACCTTCTCCGCCCTCTTCACAGCGCCCTCCTCCTCACAG
GGGTGCCCGGGATTTTTATTCTGTGCCTTCCTGGTGGCTCCTACAAGTCTGGAAGGGCAGGAGGCGCATCTCACTCCTCTGGGTCCCCTCCCCT
AGCGCCTGGCGGGAGCCCAGGCTGCATTTGTGGAATTCATGACTTTTTCTCTCCTGCTCAAGCTGAACACATTGCTGGCTCCTGCTCGGGTGGA
GCCCGGCTAATTAGAGTGAGGGGCTCCCCGTAGGGCGAAGGGGTGCGCTGTCAGATGTGGCATTCCCGTTTTACGGAGACACACGGTGTCTTAC
ACGCCAGGGACAGGTCTGAGACGCAAAGAGCCGTCGAGCGCCCTCCGCCATTGCTTCGCTGTCACCTCCGCCTGCAGCCACCCTTCCGCACGCA
CTTGTGTGTGCACCCAGGCCAACATGGAAGGCGCCATCCTAACTTCTGCCGTGAGCAGGTGGGAGGGAAGAGAGACGAGAGGTATTCCATTGGT
TGTCTGGGAAAATGAATTGCACCTTCCCCTCCCTTGCGGAGGATCAACTTTTCCCACCCCCTCCGGTGGGCACTCGCATCCTGGGGCCGGAGAC
TGAACCCGGGAGCCAAGGGGCCCCAGTTCCAGGGANNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNCGGCTGCGGGGCAGGTCGAGCAGGGACCGCCAGCGTGCGTCACCCCAAAGTTTGCGGGGTGGCAGGGCGCCCGCTCTGGCCACCCG
CCGCTCTGCGCGGCAGCTGGTGGCAACGCAAGGGCGCGGCGGGGGCGGCCGGCGCGGAGGGCCCCAGGTACGGGGCCCGCGGGCGGCGCTGTGC
GCGCGGGGCAGCCGGTCCGCCGGGAGCGCGAAAGCCTGGTCTGAGCCGGCTGGGGGCGGGGAGTGTGGCGGACAAATGGGGAACAATGCGAGTG
AGCAACTTCAGGAAGTCATTGTGAAAGAAAGCTGGGAAGAGCTCCGCGGCCAAGTTAGCAGGACACTCTAACAAGTGACTGCGCGGCCCGCGCC
CGGGGCGGTGACTGCGGCAAGCCCCCTGGGTCCCCGCCCGCCGCATCCCAGCCTGGGCGGGACGCTCGCCCGCGGCGACGCGGGCAAGCCTGGC
AGGGCAGAGGGAGCCCCCCCTCCGAGGTTGCTCTTCCCACCCGAGGATCAGTCTTGGCCCCAAAGCGCGACGCACAAATCCACGTGAGTGTTTT
CAAATTGAATTTCAATAGGAAAACTTGGGGTAACTGGTGAATTTAAAAAAAAAAAAAACACAGTAAAGAAAAGCGGTAAGGTTGGTAGACCCTG
GTGTCGCTCAGGTCCGCCTCTCTTTTCTCAGGACAGTGACAGACTTCACTTCTGTCAAGCGTCTGTTGCTCTCCACTGTGCCAGCAGGTGCACG
ACCAGGCCGACATGGGACACTTCTGAGCAGCCCCGCTGTCACCAGGAGAGGAGTTCTAGCTCCCAACCATATTTAAATTTATGTAGACCTACAT
ATACCCACGGAAGTCAGCCTTTATAAAGTCGTGTGTAAAGAGTTTTCCTTATATTTGAGCCGGGAGCTTTCTTTTTATACTATAAATATGATGA
GATCGAGTCTGAACTTAATTTCTGCAAGAGAGGAATTATCCCGGCTTTGAAAAGTTAGTCCTTTTGCTGACCGCAGGTTTGACGCTCAAGTCAC
CAAACCTTCTCAGGAAAACCCTTAGTAATATTAAGCCATCAGGTTACTTGCGGTTATATTTGAAATGTATTTTAAATATTTGTCAAGCATCGCT
GCTGATGCCTAAGGAACCTCGTGAGGGCTTGTTTTTCCTTCTAATTTGGAGGCATCTAATGACCCAAAACCGTAGCGATTCCATAGGGTCTGAC
CAGGCACAGCTTTCAAATCCAGCTTCCCTCTCTCTAGGGACTGCAGCCCACCCAGACTGAATTTCAATGCGGTGCGCTTTGCTTAGGTTACCCA
CTCACAATTTCCCACTGCGCCGCAGGCAGTATATTTCAGCTTTGAGATACCTTGTTTTAAAATTCCAGACAAAATGGTGTTGAGGAAATGTCTC
CTTACTAGTCCCATCAACTTCTGTTAAAAGAGGAAAATTTATGGAATTTGAAAATACTGCGTATGATATTTAAACTTTCATAGACATTCAAATG
CTTTTAAGGCCAGGTTCAATTTGGTTATGACTCGAGGGGTGGGGGGGACCCACATAGAAATGTCCTGGGTCCTCTTGAGTTTATTTCTTTGTTT
GAAGATGTTTGTTCAATGAGTTTTATTGTACTCATCTTTTATATGGAATTTTAAAAAGTAACAATTTCAGTATTATTTATATTAGAATGTGTCA
GAATTATTTCCGTGACAAATCAGATCATTTCGGCTATGGCTTAAAATGTACACGAGGCAAATATTCATGACAAGAAGATTCACCTTCTTACGCT
GGCATCTTCTAAAATGCACAACAAGTTAAAGAAATAATGTGTACACATACAAATAATGATGTCACATTAAAAATACTACACTATTCTTGCTTGA
TGGAATGTATCTGATTTCCAATTTCACCATGAACATATTTCATACATTTTTTACATGAAAAAAAACGTGACTCTTAAGTCTCACAGTCAATCAG
AGCTGGTCACCAGAACATTTTATTGAACTAAATGGTCATGTTTTCTTCCCCTTTTGTTTCACGGTGAGAGTTGAAGGAAGGAGTTTAGAAACTC
TCCAGTACTTGTTTAATTCATCAGTGTTCTAATTAGAGTGGTACCTCTTGGAAAACTACACACCCCCCTAATGCAGAAACATCATAGCAATAAT
CACCCACCCTCAGGGTCTCCAGGAGACCACAAGGGCTGCAGATAAAAGTCTGGATGTGTTAGGTTTGACCCTTTCGAAGAGTTTTACACAGGCT
CCTAAAGAGAAGATCAGCTGTGGCCGTTTGTAGCCATTTCCTTTGTCGAAAAACTAAGATCGCAGTCAATGTATTAGCCAAGAGGTCTAAAGCC
CTGTTGTACTGCAGGCCACTGTCTTCCTTGTTTGACTACAGACTTGGAGTTTGAGAACAGTGGTTCTTTGGTTTCGATACATTTTTTGTTCTTG
ATTTGGATGTCTGTGTTTCATGCGTGGTTAATATAGCATATTTTCAATATAAATGTCAAAAATTTTGAAATAGGAAAGAACTCTCTATATATTA
ATGTACTTATACACACACTTCAAGATTATGCATTTATTAACAGATACATGAAATAAATTCCATGTGCATATGCACATATGCACACACAGCGTGC
ACACACACAGCATGCACACAGCGTGGAGTGAGAGGCATGGGGCAGTGTGGAAGAGTTTTAACATCAAACAGACCTGAAATGAGTATTAAAGGCC
CCCTTTATTTTTAAACTTTTACTAAAACAAGATGGATTTCCCTATGTTATATAATGGTGAATTTTAGGCATAAATAACGTTTTTTGAGTGTTGC
ATAATTGTACGTATTAATGTAATGTAACTGTGGTTAACGAAGAATTCATCAAGGATATCACTGTTTTGTGGCATTTTTTTTTTCCTCCTCTAAT
CTTTGGACTTGTGAAATAATTTCACTATGAAATAAATGTTGGTTCTTGTCATATTCTAAGGGAGATTGATGTAAGTGGCTCCACTCCAGCTTAC
AGAAGGTAAACCACGACCTTTTTGCGTTCTCTGAAAACGCTTGTCTTCCGATGCCTCTGTTTCTAAGACTGACAAGCACTCTGGGGGCACTGTG
ACGCCTGCTTCTAGCGGCAGAGTTGCTGCAGCTCCTGTCCTGGCTGTGAACATTGTTCTCTCTCTCCTGTCTCTATGTTCATAACTACAGAGAC
TTCAGCTCTATTCCATTTCATATTTGTGCTGAATAATCATTCCATTTTATGGGAGAAAACACAAGATGTAAAAGCAACAAGTGACCCATCCTTT
GAAGCTTACAAGAAGAGAAACATTAATCTATTTCACGTCTTGAAAACAGATCAGTTTTATTTTGCTCAAAAAGGGCACATGTACATTTTTGATC
TAGGTCTTAGAAACGTAGAGTTTCAGAGGATCAGCATTATACACACTGTCACACACACACACACTTAAAATTCAGATGAGGAACAAGATAGGAA
TGAGGTTTTGTTAGGGACGCAGAGCACCTAAAACCAAAGGATATCGACAGTAACAAAGCTGTTTTTACTGTAGTGCTGACTGAACACTCATGCT
GGTGTCTTCATGTGGACCATGGCTTTCTTGTATTTCTTTGCAGTTTAATAAATGACTTCATATCTCAGGTTACCTTTCCACATCTCCTGGAATA
TATGTTTATGTCCTTAAAGTTTCAGTGTCGTCACTTTAGTAGCTTTAGTTTGAGTTTTTAAATGTTTGGTAATATTCCAACAAATATTTTTTAA
GACATTATGAAACCTTATGAAGTGCCATATATTACAAGTGAGATAAAACAGCAAGCAAAAGAAGGTTTGCAGAAGGTTTTTAAGTGGCGAAGTG
CGGGCCTGCCCATTTTGGTGTCTCCTTGGTGGTTACTCCTGAGAAGGGCCTGGAGGAAGAGCAACTGAGGCCTAATCTACAGGCAACTGCCAAA
TTGTTTCAGTTGACGTTTTTCCCTCTCATGTTTGACTATAATAAATAGGTAGTTGCCAGTGGAGCCTTCAGCCAACCACCTGGTAATAAACTGT
TAAAAATGGTGCAAACCCTAGGTCACAGGTGTGCGGGCCATTTGTCTTGCCTGTTAACAGGCCTGGCCTTAATTCTTTTCTCCCATGGCCATTT
CTGCCTTTGGGGAACTCACAATTCCTGTTGACTAAAAGAGCACCCTTTTCCACCACAAGCCTGACAAATCAGACGTCCACATAATTTCTGAACT
CGTTTTGGTTAGGACAGGAAGCACAGGCTCCCTTCCTGTCTGTGTTTTCCTAAGAGAAAACGGTCTTCCCTCCTTTTTTGCATATTTGGCAAGT
GGTTCCACCTTTCTCTGCACCCTCGTGGAGTGTGAAGGCAGCAGAGGAACCTTTTGCAGGAGGAAGAGGACACAGAGGCCCTGTAGCCAGGCAC
CAAGATCCCTCCCAGGTGGCTGGGTCTCACGGGAACTCCGAGCAGCCCTAGGTCCTCAAAGTCTGGATTTGTGTGGAAAAGGCAGCTCTCACTT
GGCCTTCCCGAGGCCTCGGTTGGTTGGTGAGTGCCACACCGTTTCTTTGTGTGCTTCCATGGATTGGAATAGCCATTCTGTTCTTCCGTCTTCC
CTGCTGGTGTTTCCACAGTGGGTGGCCTGAGCCCAGAGCAGCTCCCCATATCCCTGTGCAGGCCACCTGTCTCGGGTGATGGAGAGCATCATTA
TGCTCCGTCTGAACGCTCTGCTTTCGGATGGCCCCATGCTCCACCTCCTGATACCTCGTGGCGCGGGGCCACGGCTTAACGAATGGCTGAAAAT
GGGTCCTAATTAGTGGAAAAGTCCTTTGTTCATATTTTCTCACTCGAGTGTGCAGTGATTCATTTTTCTTCTGCAATCAGCTCACTGCTAAAGT
AAATCTGACTCTCTTCCCGCCATTGCACACCAAAAGTTAACTCTAATGGGTAGGAGGTTAGGTTTGTTGAGAGAGCAATGCAGTAAAAAGAGGG
GATCCAATGTGGTCTTGTCTGTCTGGTCTTCCTTTCTTCGTTTTTTCCTCCCTTGTCTTCTCTGTCATTCCCTTCCCTCCATTTGCCTTGCCTT
TCCTGTCCTTCCCTTCCCTTCCTTCCCCTCTTTCTTTCTATAATTGGTGGGGGGTTTGCACAGACTGCCAAAACACTAACAACTGTGTAAAGTG
TTTTTGAATGGCCTTACACATATTGAAGTAGATTTTTATGCTCCATTTTTGAGATCACACACTAAAATCTATACCTTTAAAGCATTTTCTGTTA
GTTTGAAACTATTTGAAAATGAACAATGTGGTTTAGATTACAGTCCTGTTCTGAAGCTACGAGTTCCACTATGAATATTGATTTATCAGTTTTT
GACAAATTTTTGTTGTTATACCAGATTTTCACTGGCAAACCTAGAGCAAATAAAATTCCACATAAGATACTTCCCTAGACCTAATGGGAAAAAT
GTTTAATTTAGAGTCTTTAGCACAAATGAGAATGAGGAATTGACCTTTTGTAACCTTACTTCTGAGGCACTCTCGAAGTGTGTTCAGTGCTTTT
AATGGAAACTAGAGAGAGCCACCAACCCCCTAGTGTGAGCCCCACTTTTAACCGGAAAAAGTGACCTTTTCCTCCTCCTTTGTGCTGAGTTTTG
CGTAGGGCAGAAAATTAAGCTGATATTCAAAGAGATTCACTGCAAAAACATATTGATAAATCGTATATTCTATTTCATTAAATTAAAACCATAC
TGCTAATTATCTCAGGTTGTTAAACATAAGGCAATTAATTATCATTTTAAAAGTTGGTAGGAAGTTGTGAGTACTTTTGCAGTATGAGTGTTTT
CCCGCTTTAGTATGAGGTTGTGTATGTTTGCTTGAATTTACAGAATTTTCACTTTAAGAGCAGACAATGTTTTGTTAAAGAAATGAAATTTGCT
AAAAAGGACCATGTAAAGTGAAACATTAAAAATAAATAATTTCAACTTACTTAAGAGCTGCAGAAAAATCTGATTGCTGTGTTTAAAATGAATT
TTCCCACATTTCGCTCTCTTATGGACAGGAGCATTTTCTGTCACGTTATAAATAAAGACATGCCCATTTTTTGTACCCCCACAAATGAGGAAGT
TGTAAGCTCTCTGAGGTTTTACTGATGAGCCCCCTCCCCCTGGGTTTGCATGAAGAGATCATAGGCCACAAATAAAGGACTACAAAATGGGGTC
TAAACTATCCTGGTGGGGCCTGATACCCACGTTTCGCATGGACCTTACGATGTGATGAATGGTTTTGGCATGAGTGTCTTAAGAATGCTTCCAG
ATTCGGGTTACAGGACAGCCAGCGCTGAGCTCCCTATTGCAGAACAAAGTAGGAATCTAGAACTTTCTTGCTAACAGGATCCAGCTAAAACACC
AAGTTAGATTCTTAAATGATGTTCTTTTCTGTCATTATTTGATTGTTGTCAGTAGCAGTAATTGTTACCAAGCCATTGATGCTTCTATTCTTCC
CTTTGCCCTTCTGAGACACAGCTCATTTTGACTTCAGTGGAACCCCTCGAAGGTGGGGTGATGAGCAAGGTGAATTTTCAAAGTAAAGCTACTA
AGAGACCAAACTACAATTTAAGGAACCTGATTTTTGAATCAAATTCCATATACTGTGGGTATAGTTCAACATAGATTAATTTCTTATAGTTATT
ATGAAAAAAATCTCATCTTGATGATAGCTGATAATTTTGTGGGTGTCGTAAACAAAACAGAGGTCAGAATTCAGTCCCTTGGGGAAAATTTCCA
ATTAGTAGGAAACCAAGTGGCCTACCTTAGTTTGAAGACACCCATCAGGATGTCTGCACCTTTTCATCCTCTCTGGAGGAAAGACTAAATACCC
ATTATTGTATATAGGTCAGGCCAAAGCAGCCTTTTATATTGCAAGGAATAAGAGGTAAATAGATATATGTGCAACAATGAATCCCCTAATGTGT
TTACTCTAGAACACATGTTCTTTCTGTATTTATATGTAGATTTTGTAGATCTTCTCTTACCACCTGCTAATGGTAGATACTGTATCTAAATAAG
TTGAGGAAAATTTATAGTACCTAGGAATGTGTCCTCAGTGGGCCAATCAATCAATCATGACTTCAGGTTATTTTTAATAAATATACACGTATGG
GTTCATAAACAATGGGATGTTCTTGTGAAGATCTAAATAATTTTACTTCTTTGGGACTAAATAAAATATAGCTTTTGCCAAATAAACTCACACA
AGCACTTATTTTAATAGAAGTCAAATGGCTTTGCAGAAACTTCAGTTTTACAGGTGCATTGTTTGAAATGTTACGGGTATACAAGTGGATTTCT
CTATTATGTACAGTGTTAAGTTTGAGTTTCAAAATGTCCACCTGAAATGATTTACTTGTACGTTAAGATAATTTAACTGCTAAGAAGGCAAGAT
AAAGCATTCTTTCTGACACCATATGGCCTTGCTGAGGGAAAAACTTACTGTTATAAGTTTGTGTTTATCTCTCTTTTTAAAAAAAAATGAAGAA
AAAAACGTTTAAAATAATGGGAACACAGCAGTTCCTGGGGTCCTCTGTCTCTTTATCTTATTATAGTAAATTACCAAAAAAATAATGACCTGGG
GCATGTCTGTGTGGACCCTTCTTTTGGAGGCAGTTTCTGTGTTTTGTAAAGCTGTAGGTTCTATTTTCATTGCACTTCATATTGCTGCACAGCT
CCTGACCATGCATGAAGGTCCTCTCAAATCGGTAAGAGGGCAGAAGAAAATGATTCTAAACTTAGATTTTTTTAACTTAAGTGATGAAGTGTGA
AACGCCATTTATATTTGAGGAAGCTACCTAGGAAGTGGCTCATGTCGATGGCCCAAATCAGAAGAGGGCCTGTAAAAGCTTCTATCAATTTTGA
CTGTGTATGCTTCTACCATGGCGGCTCAATAAACAGCAGTATTAGTTTAAGAGTGGATGGTACAGTAGTATAGACGGGAAGCCTCTCCTCTCCG
TGTGAACCGTGCACCCCTATGAGAGGGTAGAGACAATACAATATGCCTGTAACGTCAGGACAGACACTCATGGCCAGCTTGAACTCCAGCCCTG
GGCTTCTTGCAGCAACAAACGTGAACACAGAGGACTGTCTCCAACTCCACTTTCTCTATTTTTAAAACAACTTTTTGAATACAGTATCTGCCAT
CTTTTCTTATACCTCACTTTGAAACAGCTCGCTCCACTGTGGCATTTAAAATGTTCTCTTTCTTTTCCCTCTGTATCAAATACCTCTTTACCAA
GAAAACATTCAAACAGCATAGTTTTTAACTGTATTTTGAAAGGTTTCCTTAGTTCCCTTTGACCCTTCCTCTTTTGCATATCAGTTCCTGGCCA
TAAAAATAAAAAATGCTAGGACAGAATTGCACATCTGAGCTGATTTGCCCTCAAAAAGTTTCACAGTGGAACAAACCGCAGGAGGAGTTTTCTG
TGGCTCAGTTAAATGTCGGGGGAGGGTGGTGTGAAAGCCAAATTGGATTCCTGCTTTCCTGTTTAAATCTTGTTTTTCATTGTTATTTGCACCA
GCAATACTCTGTGGAATAATCATGAAAATGTGTAGATTGGCAGCTAATTTTTGAAAAATGAAAAGAATCAGAAATGAAATAAGAGTGCTCGGAA
GTTTTTATGTTCTCTCAACCTGTTTTGTCAAATTGTTACGAAAACCTATAAGGTCTCTTTGACTAGATACAAAGACTTTGCACATTGCCTTAGC
TTTCTCTTGAAGCATTTCCTTTTTTAAAATACAGTGTAATTCACAGTGATATGATAGATTTGCAAAAGTAAAATCTACCAGTCTGAAGATGAAA
GGACTTGTCTCTTAGCAGGAATAATGGGTTTTATTAAAGAGGTCTGTGACCTAAGGCATTTTAAATAAATTACAGGCTTGGTCCCTGTCTCCCC
CATGTATCTACTCCCTTCAATATAAGCATCATTGAGTATTTAAGGAAATAACCCCAAATGTAACTCTAGTGTAGCTTCACTTGTCAGGGAGGAA
AAAGTAAATAGCATACATTTGGCCAAATAACCAGAACTTTACTGTAGAAGTTTTATGATGAAATTTGCCTTTAGTGCAGAGTATTACAAAGATC
ATGTTTAGTTTCTAGCAGTATATAAGTAGCATCCATCCTTATCTGTCATGCATTTGGAGTGTGCGACCCCTGCACTGGGCTGCAACATTCTGAT
GGGCAAGAGTGCTAGGGACAAAGAGGCATCACCATCAGACTGCACGGGTTCAAGTGTCAGCTCTGTGGTTGATTAGCTGTGTGACCTGGGGAAA
GCTATTTCTCTTAGCCTTGGTTCTCTCATCTATAAAATGGAGATAATGATGCAGATGCCTTGGGTTTAATTGGGACAGTTAAAGACACATTTAC
ATATTTAGCAAGTAGGTGTTGAATTCTAGCTCTACATTGGACACTATGCCAGGTGCTCAAATAAACAAGTGGACAAGACAGACAACACCCATGG
TCTTATGAGGCTTAACCATTTGCCTCTTCAATGCCAGAAACTTAGTAGGTTGATTAGATAAAGCCAGTGAGTACCAGTATCCTTTTCTTTGCAG
CCTTTTCCTGGCACACTAAAAATACTCAGTACATATGAAATATCACTGGACAAAGAATCCCCCTTGGAGTACCAGTGGAGAAGGAAGGCATTTG
CTTAAAAGCAAACCAACAGAAAGACATTGTAAGGCAGTTGTTTAAGTCTCAGAGACCTATAATTTTTTTCTTTTTTCTTTTTTTTCATCTCGCT
CTGTCGCCCAGGCTGGAGTGCAGTGGCACAATCTCAGCTCACTGCAAGCTCCACCTTCCGGGTTCATGCCATTCTTCTGCCTCAGCCTCCCAAG
TAGCAGAGACTACAGGCGCCCGCCACCACACCTGGCTAATTTTTTGTATTTTTAGTGGAGACGGGGTTTCGCCGTGTTAGCCAGGATGGTCTTG
ATCTCCTGACCTCATGATCCGCCTGCCTCGGCCTCCCAAAGTGCTGGCATTACTGGCATGAGCCACCACGCCCGGCAACTACAATTGTTCTTAA
AGCTTGTAGAATTACTGTGTGCTACCAACAGACAGGCTAATTTTGAGTGACCCTCAGTACTTTGTACAGTTAATTTGGCACGCTGTGTACTTAG
TGGCTTTTTAACAGCTATAAATTTGGGCTGCTAGAAAAGTAGTAAAGTTGTGATTCTTGACAGGCATCTATCTGCATTTTCATTTTTACTTCAT
TTGTCTAGACTCAGCTTGTCAGAATTATGGAAGAGACTCCTTGTGTCAGGGCAAGCACTGTGAAGAGAGGTATTCACTGTCAGAAAAGAGAGGG
GAGCTGCAGGCAGCTCAGAGGCCTGAGACCCGCCTCCACAGGAGCCCCAGCAGGTTCGGTGGAGCTCTGGCCACACTCTCCTTTGGGATGCTGA
AGTCAGAATGAGTTCACTTCCCAGCCAGTCTTGCCAAGGCTCCTCACCTGGAAGCAGCAACTGCCCAGGGCTGTTGGATGTTTCTCCCCAGGGG
ACAGCCAGGTCCCAGTCCCGCCTCGGTGTGGAAGGAGGAAAGGCAGGGTCCAGGAAGCTGTTTCAGGACAGGCCCAAGGTCCCCCAGGGATGCC
TTTCAGGGTCAGCGGAGGCTGTAAATCAGCAGGGCCCACACGGCCTGGAAGAGGCCCCTGTGCTGTCGGCTTGCCCGGCTTGCCCGGCTCCTAG
TCCGGCTTCTGCTCCTCCTTTGTAAAGTTATGGATATGCTAATAGTTTCCAACTGAGACTAGGAAAGTAAGTCCTACTTGACACTGTTTGGTCA
GAAAGAGGGAGAGAAAGGAGAAGGACAGAGAGAGACTGAGAGAGAGACAGTCTCAGACAAAGGGAGACGGAGGGAGGGAGGGAGAGACAGAGAA
AGAGATGGGAGGTAGGTGTGGGAGGAGGGAGAGATGCAGAAGGCAGAGGAAAGACAGACAGAGATTTAGACCTCCCAAGTCAGTGAGCAGTCCA
GAGTTGGAGTGGAGGGTGCCTGGTGGCTTGTGACTGCAGACTCCACTCCCCGCTCCTAGAGGCACAGCCATGGACAGCTTCTGTCACGTTGGCC
CTGCACTTATCTCTGCATCTATTTCCCCTTGTGCAAGATTCAGAACTGCATGCTCCAAAAAAACAATAAAAGCATTCATGTTCATAAGAATTGC
ACACGTAAAAGGTAGTTTGCTGATATTGTTGTATTTTTTACTATCGCTTCTTTTAGCTCTTGCCTGAAATTGTTTGGGTTTCCCAGGCAAAGTA
GAAAACTGCGGTACGTTTCTGTGAAATAATTATTCCTTCTGGCATCTCCCTTTACAGACCTACTGATCTTGATTTTTCATTTAGGTGAAAGTTT
GTGAAAACATGCCATTAGCTTGCTTTGTGATTAACTCCTTTTACTGAATGTGACCTCCTTTTAAATTGAGGCCATATCAAGCTTAAATTCCATA
TTTTACCCGGCACTCTGCATTTCTTCCATGTGGGAGAGGAGGGGCTCAGTAAGTGCTTTGTAAAATACACAGCCGAAGTGATGCACGTGCTAAC
AAAGGAGTGTGACAGGACTTAAGTGCCCTTCTAGACACTTCAGGCTCCCCTTTGTAAGCTGTCTTGGAAGAGGCCACATTTCCTTTCCCTCAAA
CAGTTTCTCATTGTTTGATTATTCTTTTAGCCTTTCTCTGGAAGCAAAGCCACTTTTACGAGAAAGTCACTGCTTTTTCATCTCAAGAGATGCA
AGTTTGGAGTTTGGGGAAGTTTTCAGGTGCCCGTCAAGTCATCCTTTATGATGTCAGACGAGTCAGCCCACAGAATTCACAGGGCTCAGTGCAG
ACCGAAAACTTGAGGCCTCTTGTTCAGAAATTATTAAAAATTTTGGTGAACATCACCCCAAGCAAAGAGATCCCCTAAGCACCAGCCCCCAAGC
AACTGCACTCATAAGCCCATGAAGCCCCCTGCTGTCAGAAACAATGTGGTTGAAATTGTGTATGCACTTGGAAGTGAGATGGATTGCAAAACAC
AGGTCTCCATGCTGGGGCAGGAGTGGTGATAGGGCATGGAGTGGAAATGTCCAGCAGGCCCACGTGCGAAAATGCAGAGCTCTCTGGCTCTTGC
AGACTTGGCTGCTGACAATAGACGCGCTCCACGAAGGTGCTCGCTGTGGTGTGATCTGCTGCCCACCCCTAGCTCCCTCCAGGAGACTGGTGCG
GGGACTGTTTGCAAATGACTGCAAAAGTAAGAAGGTTCCCACAGAGCAGAGCTTGATTTGGGGACCAGCCGAGGGCAGTTTGTCAGGATTCCGG
CTTGAAACTGTTCTCACATCTCACCGCCTGAAAGGACGAGTGTGTCCAGAGGACTTAGCATTGATCACCTCTGTCTCCATGCAGCAAACTCAGA
GGCTCAGCCCGCATTCCACTGGAAGGGCGTTTGCCAGTGGTGTTGGTTGGAAGAGCCTTGACTTTGCCTTAGGAAACATCTTTTTTTAAGAATT
GAAAATAACTTGAGTATGCAACAGTAGGGCATTTGTTATATAAATTAGTTGACTAGTGTGTAGCCAGTAAAATGATGATGGTGGTGTGTATTTG
TTAAATAAAAAGATATGTGTGGTATTAAATTAAAAAATATTTTAAAACAACATATTTGTAATCTGTTTAGTGTCCTCTTTTTGTAAAAAGTACA
GAAATAAATATACAGAAAAAATAGTAGTCCTAAGTGGTAGAAATTATGAGCATTTTCTTGCCTTTAAAAAAAGTTGTAAAAGATTGTATCATTT
ATGTAGCAAAAAGTTTTAAGTCAGCATTCTAAAAATTTCGTGTTGTTATAGTTGCTGTGACAAGATTTAACTTCTGTATGCTTCACCAATCAAT
ACAGAGGTATTTAAGACCCGGTGTGTGATAGGCCGCGCTAAAATACTATACACATCTTCAGAAAACTAGAGAACTAACTTCTAACTTCCTATAT
TAGTGTGGCACGGCTGTTACAAAGATTTTTCTCATTTGAGTCTATCTTGCTTCTTTATCATTGTTTTGACAGTTTCAGAAGAATCGTGGCTTTT
CCCCTTTTTTACAGTAAAGGTACCTGAGACTCTTGACGTATTCCTTTTTGGAAATGCTTGTGCTGGTCACATGCTTGCATCTGGGCTAGTGTGT
CTGGCTTCCGTGTGCTGGTGGATGCTTACTCTGTTTTCTGAAATACTTTTTCTGTACAGTGGCGACTAGCTCTACTCCTAAGCCACACACCTAC
CTTGAAAATTCATGTCACTTTTAGAAATAGATAAAAGCCCCTCCCATCCAGAAAAAGTGACTATCATGTATATCCTCATCATGACTAATACTGA
TATTCCTGAAATTGAAAATACATATTCCATATGTACCATAAAAGGTATTAAAGATATATGGAGTGATAGATATATTATATATAACACTTCTACC
CTCACAGTTTTCAGCCTAATTGAGAGGGTAAGATCCCTGAATCATCCATCAGTTTTTCAGGTCTCTGCTGAAAGCAGGCCACAGCTCAGATCCA
CACATCTGAACCAGACACAGAGGTGCCCAAAAATAAAAAGGGGGACAGGGGGACAACCTGGTTTAGAGTCAACAAATAGACTGCATTTTCTGGT
TAGTGAAGGAGCTCTCCTGAAAGTCATATACCAGAGCATAAATGAGCAGATTTCCTTGAGGTCACCTTCTGCTGGCCATAGCTTTCTTATCTGT
GGAGCTGCCAGCTGTCATCCACTTTGGGGCACCTGAGACTGCCGAGCGGCAGGCCAGGACCCAAGTGCGAAACACAGAACACCTTTTTGTTTCT
ACTCCACTGATGCTGGGGTTCTCTCCCTGGTGTTTGTGGCTCGTAGTACACTCTGTGGAACATTCACTATGGTCATCGAAGGGCAGCATCTTCC
CAGTTGTTTCTTTCTTTTCTTTTTTTTTTTTAATTTAAACCGATCTGAGAAGCCAGCCATCTGTCAGCAAAACAGGAAGGCTCGGGCTCTCTCC
TGGGCTCGTTTTGCTCCCGTAGTGAGCGTCACTTCTCCCCGTGTAAGAGTGCTGGTGAAGGCTGAGGCAAGGGCCCAGAAAGATTGAGGGACAA
AGACAGGAGCGCCCGCATTGCCCATCTCCCAGGCTGGAGGTGTATTCATTATTGATGGAGGTAGTGCAGTTGCTGCTCAGATATGCAGCCCTGC
CTCGCTAAATCAGACATTCTTCAGCAAATTCCTTCGTTTTTTGATTGCTGATTGTACCCGTGTCACCAAGCTGACTCAAGGTTCATCGATGCAT
GCTCAGTAAATTAGAAAGAACATAACTATGGATCAGCCAAGAGAATGAATTCTGTGCCTACAATGACCCAGGGCCATTTAATTTTCTGCTTAAT
TTTGTTGCAGTCAGTTTGCATTTTGGGTTATTATGCAGTAGGAAATTAACAATAAATAACAAATTTGGTCCTCCTGTGCTTGTAATGATATTTT
TATAAATCTTTGTAATGCTGTTTTTAAAAGGATCAAGGTCTGTGCCAGTCTGATACTCCAGCAAGTATGTGAGGAGGAAAATGCATTATTCTTG
CTAGATAACCTTGTTGTTAAATAGCATAGCGGTTCTTTATCTCTCTCTCTTTCTCATATCTTATTAGTATTTTTGCTTTAAACTAAAATCCCTT
CCTCTCTTTCTCAGATAACCTGACGACCATGGATGCTGATGAGGGTCAAGACATGTCCCAAGTTTCAGGTCAGACCTTATGAGATAGCTGTGTG
GGAAGTTCATGAGAAAAGCTTCCCTGGGGCCGGAAGTCACAGTGCTTGGTATGCTCATGGGGGAGGAATAGGGGCTATTCTGCAAAAGAAAAGA
CCATGATGGAATTTGCCTGAGTGTTTCCTTCACCTGTTACAAATTATCTCACTTTGAGCTGAACAGAAAGCCTCCAAGATGAAATTAGTTTTAC
TGTTAAACTTCAGGAAAAAAAAACGGGAAGAGTTAAATACATTTTTGTACTGTTGGAAGGAAAAATGGCTGATTGGTTTAAAACCCAAACACAT
GCCAATGATGGTACTTAAAGAGAGAGAGAGAGAGAAGCTTGAAAAACATAATTGTTGGGCACAGTCATGACTGTTTGTTCATTAAGCATGGACA
CAACATTGCTCCCCTTTGCCATATATCTTTTCAAGCCGTATTGGATATAGCTCTTCTCATCCAGGAGACCCAGGAAGTGGAGAAGTCTGTAGTA
GGAAAAGCCTAAGGGTAGGTCACAGACTGTGACCATTTGGCAGCACTGAGGGTGGACGGCGAGCCAGTCCAACAAAACCGCACAGTTCCCCAGT
GCATGGACATAGGAAGACAGCTTTCTATCTGGCCCTGTATCCAGAGGCGTCAGCCCCAGTAGCAGCTTTCATGGACTTTGGGGTTTTCGGTATT
TCATATTTTTGAGCCTCACAGACTCACAGCCAGCCCCAGAGCCTGACTTATATTTGAGAAAGTTCTCAGTGGCACCTTGCCTTGGCTGAGCGCC
CTCGTGTTTTGAAGTTTCTATGGGATTCTACAAGTTGGTGCTCCTGATGAAGACCAGGACCTATGTGTGGCTGCTCCCCTGCTTGGTGGTTTCC
CTGGGGAAGGTGCAGGAGAGGATCTTCTGAGTTCCATGGAACTGGAGATAGATCTGCCAATCACAGGCTTCCTTCTCCACCACTCCTCAGCCGC
TCTATTCATGTTTCAGATTTTGGACTTAAACTCTCCCAGGTGCAAAGAACAAACAAAAGGCTAGCTTATTTTTCTTTTAGAGTGAGGCTTCGTA
TTTATTACAATATAATTGCCACATTCTTTGTGTAATTCTCACATTTATATCTTAAATATAATTCTCATGAATGAGAATTATATAATTCTCTTTT
TGTATATCATTGAATATTTTCACTTAATTTTTAATTTTTTTAATCGTCACAAAATAATTGTGTACATAGACACAAAATAATTGGGTACATAGTG
ATGTTGTGATATATACAATGTATAGTAATCGGATCAGGTAAATCAGCATATTCATCATCTCAAACATTTATCGTTTCTTTGTATTAGGAACATT
CGACATCTTCCTTCTAGCTATTTGAAACTATATATTATTGTTGACTACAGTCATCCTGCAATGGTGTAGAACACTAGAACTTATTCTTCCTACC
TAGCTGTAATTTTGTCTCCTTTAACAAATCTCTCCCTATCTTCCACTCCCCCGACCTTTCCAGCCTCTATTAGCCTCTGTCCTACTTTCTACTT
ATAATGATGACAGCAGCATTTGTTAGTTTCCACATGTGAGTGAGAACATGTGGCTTTTTAACTTTTAGAATGTGGTATTCAGGCACTTCATGGT
ACAGTTGGTAAAAGTGAAAATGTGTCCAAAAGTTTGTGATTATCTATATAAACAAAAATGGTATAAATACAAATATCAATTTTGCATTGAAGAA
CTTACCTTAGAGGTATATTCTCACAAGTGCACAGAGCATTTAAGCATTTCTTCACTGCAGCATTGTTATCAGTATTTTAAAACTATGGTACATC
CATGTACTTCCACATACAGCTCTTAAAAATAAGGAGGATATGAATGAACTAGTATGAAAAGAAGTCCAAATACATGTGAAAGTGAGAATAGCAT
GGTTCTGGATGGTATGCAAAGTATGATCTCGTTCTTTTAAAAGAAAATAAATTACATACACATACATATTTTCTATATGCTTGCCCATAACGTT
TAGGAAAATTCTTGGGTGATATTTATTAACCTGGACTTCCTCTTGGAAGACTGATGGTAGAAGGAAGGGGACGAGTTAGGGAAGAGGAGGAGAA
GGAAAACTTTGCTTTTCATCTTCTACCTTTTAGCATTATTTGAATTTATTTTCCTTAAGCGTTTACTTTGTTTCGTAAACAAAAAAGCACAAAA
ACAAAAAACGAGTTAAATGGGAAAAAAAGCAGTTTAGCTCTTTATAGCCTCTCATTTGGCTTCGCCAGCCTCTCACTGCAGCCTCAGAGAGCTG
GTCTGGGAAACACTGGTAGATGAGGACTGTAATCCTCACTCATGGAAGAGGATCTCATTCACTGGGTTTGCTGACTGTGACTAGAAGTGATTAG
GGTGTCAAAAAACCCAAGCATGTTAAAAATTTCCAGAGGCCAAAAAGATGCTTTCATTGTTCTGCTCTTCTTTTCCTTGTCGCTTTCACTTTGG
GTAGCTTCTAAATTGGTATTTTGCATGGTGCATTTAAAGAAAATGAGACCCCTTTGGCCAATGCAGGAGTCTACACTCTGATATTCTAGAGTCA
AAGCTGAATGCTGACACCTAGGAATTCATCTCTAGAATGTTTATATAAGGAATAGCCCCTCAGTATTCCGATCTCGTATCTTAGTAACGAAACT
AACAAAAGCCTGATTCTCCTCTGCTAGTTTTCTTGTCTTTACCATAATACAAAATAAGTAATTTCTTCTGCACCCTGACTGTTCAAAGGATAGG
GTAGCTGGGGGCGGGGACAAGAATGGAGACCTTATTACATAAGACTTCCTGAAAAAGGAAACTCTGTTTTTGTTTGAAATGATTTGGTCTGAAA
TTTAGTTTGTGTACACTTACCAAAGGGATTCCTATTTCTAAAACACTCATACTGCTTTTGATTCCTGTTAACCTTTGAGCACTCTACGTAATGA
TGAGAGCACTTAAAGAGTCATGTCACTTTTAGTAAAGAATCAAAGGATACTTTTTCTACTTCTTCGAGTTTGATCTCTGCTTCTCCAGTTAAAA
CCAGTATTTGTTTTTTTCATTTCTAAAGTTGGAAGAAATGACAGTTAGTTATGGCATAAGGATGTACATTTAACCAAATAGGAGTTGACATTCT
TGGTAAGAAATCTTACCAAGATTATGTTATAGATTATAAGAAATCTTAACAAGAATATGTTCCTAAATCATCCTCTTTTCCCATAAAATATTAA
AGTATCAGCAATTTCATAGGATTCAACCTAATGTATGCGAAATGCTAGATAAACAGATAAATACTTAATATCTGGCTTTTTTTCAAAGCACTGG
GTTATTTGTTCCTTGAGATTTATCCTAAATGTGGGCTATACCCTGGTTTACAGTGTCTCACAGATGTGTAGTAGTAGACACTCCATAAGTGTTT
ACTGACTTGAATCCACAGGGTACTGAGAAAATGCTACTGATAGACTTGGAGGAGAGCATATCTAAAGCAAGCTACCCTTTCCTTTAGGGCACGT
CTCACTAATTCTTTGGGTAAAGCGTATTTTTCTTCCTTTTGTGTTTTTGGCAGTCTTTCCAAAAATACGTGTTATACCTATGCATTATTTTTTG
GTTTGGTTTCTAAAGAAAGAGTCAGCCGGTGGGAAAGTGAAGGATGTGGGAACTGAGAGATCTGCATCAGCATCCCACCTCTACCTCCCACGAT
GGGACCTGAGACAGTTATTTTTCCCTCCTCGACCACTATACTATCATCTGTAACACGAGGGACTTGAGCCAGTTGATCTCTAAGGTTCCTCTGG
CACCTGTGACCCTAAATAGATATTGGATATTGGTTTAATGCTATTTGTAGTGTGTTTTTTTGGGGATATGGAAACCAGAAGTTTGTTTCCATAA
ACATAAACATAAACTGTATATATCTAAAGGATATGGAAACCTTTAGATATATATAATCTGCTTACGTAAAGAAGGTTTGTATATATTGCAGTGT
CAATGGGAATATTTTATCAAGTTAAGCATAGTAAATCACATTGATTAAATGCTTTGTATTTACCAAACATTACCCAAAGTGTTTTCTCCTTTCA
ACCTCACAAGGACCCACAGAAGAAAATACAGTTATCATTTCCAACCTGCAGGGAGCTGAGACACAGAGAATTTAAGCAACTGACCGGAAGTCCA
ACAGGGAGTCAGAGATTGCTCTGGGGTGTGATCCCCACTTGGACCCTACAGTGGAAGCTTCTCCACTACTTTATACAGTTGAGATTCTATATTT
TGAGCTTGTATTTACCCAGAGAATTATATCCTCTTGGGCAATTGTATATAATAAAACCTCATGCATTTAGGAGAGGCGGGATGACAGAACTTTG
TTGACTGAATTATAATCTACTTGAGAAATTATTTGCTTACATTTTATAAGCTAATTATACCATATCTCATCCAGTTTTCCCAGAACACTTCTCA
TAGGTAATGCTTTATTTGAAACATAGGCCATAGGTAAGTTAAGTGTAAATGTGTATTTTTATAATTTAACCAGAAGTTTATTTCATTTTTCTAA
ATAAGTGAAATTGTATTGCATCTTCTAAATTATTCTATTTAAACACTTGATGTCTTGCTGTCTCCGTCTCTGTGTGTTTGCATGTCATTGTACA
TCTTCTTAGGAAAAGTGTGGGAGCTTGACGCAATATATACCTTATGTTTCTATGTGCATATAGTTTACCAAATAATACCATAAGTTTACTTAGC
ATATTAGAATCCATGCACATTATTTTTATTTTATCTTCACCGCAACCCTGTGGGATAGACCAAAATCATGCTTTTCAGCCTCCTTTTTCCACTT
GAGGAAAGGAGTCTTAAAAAAGGGACCAGTCTCATGTTCCCATTCGTCTTACAACTAATTGGTCAAGCCAGAAAGCCAGAACTATGTCCTGGGT
CACTAACTCCTAGTCACTGTGTGTTAGTATTTGAGATGCCTGTTGGCTTGATTTAGTCATTTATTTTTTAGTGTTTTATAATCCTTCCATACTT
TTACATTTTAAATGGTTAACCAGGCAAATTGGTTTAAAATCAGTGCATAAAAATACTGTGCCTATCATGATGGGTTTCATGAAGTGATAACTTT
TCATCATGGAGATCCTCAGCTGTCACAGAAGATGAGGGGCCCTGGGTACAGAGGCTCACGTGAGGGATGAAAGTCTCAGCAGCCCGGACTTACA
CTTTGGGGCTTTTAGGCAAATCAGACAACCTCTTAAGAACTATCACTGAGTTCAGGCAAGGCCAGCTTGAATTAACACAGGGCCCTTGGTGGGC
ATGTGAATATATCTCACTTCACTACCATCCAGTTCTGACTCTTTACTAGATGCCCCTGTACATACCAAGACTGATTTTTTATTCTCCCTTCTCC
CCATGTCGTTTCTTCTGCATAGAGAGTTCCTATTGATCAGTCTCACCCATGGTATTTTAGAATTGCGATCCCTACTGTTTGATTATTCCTTTTT
CTCCCCCATGTTGAAAAAAATAAATGTCCCTGAGATGCAACATCAGGGACACTGGAGCACTGACATTTAGTTCAGTGCAGGAACTGAAGGCAGA
TGTAATTCTTAAGAAGNNNNNNNNNNNNNNNNNNNNTGTAGTGGATCTTGTTTTCTCATAGATACAGCAGTTAAATTTTTTAATAAAAGTAACT
AAGAGTTATTTGGATGTATTTTAGCATGCACTGAGCGGAAAGTACGACATTTCTTCATTGGGTAAGTCCTGATTCTTTATGATCCTCACTTGGT
TCCAGGGCCCCATGCATCTAAGGGTGTCTCAGAGCATCCTGCAGTGCTCCAGCATGATCGCAGGGAAAAGCTATAGGAGGAAAAGAGTCAATAA
AGTTTAGTTTCTCAACCTCCCACCTCCACCCCATAATAATGACAGCTGGTTAATCATGAGACGCGTGCACACCCCACACGCCCTGTACATGTTT
ACTCATTGGGATAGCATGTCAGGCCAGAAGGCTCCATGGTCATTTCTATGAAGGTACTTTAGCAGGTCTTCAAGAAGGCAAGTGGCCTGGGTCC
CTGCCTCCCCCAAATTGCAAGCTCCCTGCTTTATGTAGGAGACCTATGTGTATATTACAGTTCTGTGTAAGATTATTTTGTTATTCTTACCCCC
ACACCCACCCCCCAACCCCCCGCTGCCACCAAAAAAAAAAAAAAAAAATTCCTCTGACAACCTTCATAAAGTACCTGGGAGTTTGAACACCATT
GCTCTAGGAAGTCATCTTATACAAAAATAAGAGTTGTGAGGTGGTTCATATACCTTCCTGCGTTCTCCTATTTGGAGTTTTTCCCCATTTATGA
AAGAGGTGAAAACGCTAAGATATTTAGCAATTATTACTTTAAACATTTTCTATTTATAGGCCGGGCGCAGTGGCTCATGCCTGTAATCCCAGCG
CTTGGGAGGCCAAGGCAGGCAGATCACGAGGTCAGGAGATCGAGACCATCTTGGCTAACACGGTGAAACCCTGTCTCTACTAAAAAAATACAAA
AATTTAGTTCGGCATGGTGCGGGATACCTGTGGTCCCACCTACTCGGGAGCCTGAGACACGAGAATGGCTTGAACCTGGGAGGCCGAGCTTGCA
GTGAGCCAAGATCGCGCCACTGCACTCCAGCCTGGGTGACAGAGCAAGACTCTGTCTCGAAAAAAAAAAAAAAATTCTATTTACAGCATGAAAA
TAGTAGTGACTTAATGCACATTGCCAAGGCTTTAGCATAACATGAACACTTTCACTCAATGTCTCTCTGGCCTTTTGTTTTTCCTTGGGAAATT
CTTATAATCCTGCTCCGTCTTTAACTATTCATTTTGTATTGGCTATCCAAATATACCCAATAAGCTCTTTCTGAAAATATGCCAATTGTGGGTA
ATTACAGCTAAGCTGGAATATTAAATTGTGATGTCTGTTTTCCAGAGAATGAAGTAGTATTCCCCAGAGCATAGGCTTGGTGCCTGTGCAGGTT
CTATTTTAAATATTCCAGGAAGGGTTGTTTTATATACTGAGGATGATTTTACTGGTCTTGCCAGTCGTCTGAAATGCTGGTATTACTCTTGTGG
AAGGTTTATTCAAACAAACAAGGACATTTCACACAATACCTAGTCATGTTTTTCAGACATTTTAATGTTTGGTTCATCATTTGCACACACTCTC
AAAAATCTAGGTTTGTCTATGTGTTCATATCATTTTGCCTGTTGCCAGCTCAGTCAGCAGGCACACTCTCCCAGGCTGTTGCTGTTTTGTTAGA
CTTCTTCAGGACCTTCATCTAAAATGGTCTTCCACACGTAGCTATACTGCATAAGTTCACATCATCTGTTTCTTGCATGTGGGTTGTGTCTCAA
CTCAAGTTTAAGTTAGATTTGGAAGGGCGGAAACTATAGGAGTTGCAGCTTCAGTGGAGAAAAGAGCATTTCCTACTAGTTATGGCTTCCCAAG
GAAGGTTAGATTCCTCAGAGTAGGAGTGATTCCCCAATGCTAGAACCTTTGGTCAAATATAATTCTAATCCAGTCAAAATAAATACAGGTATTC
TGTAAAACCCGATTTCATTTTGTAAATCCTACTTTGTATAGTATAAGCAATTTTTGTATTTGTGTGGATTATATTTTATTTTCCTATTTCAAAG
AGAAGAATTTGTATTAGCAGACTCCCTTTGCATGCGGAGAGGGGATCATTTTCCCAGTAGGCATGGGGTTCCCTTCCATTCCTTGTCCAGTCTT
CTTTTCCCCACTAAGTTAAGTCAAACTAAGCAGCTGGTAAGATATTCCCTGGTTCTTGCAAAGAAAGTGAGCAGATGGCAGAATGTATAGCTCT
AAGCAGAATACCTGGTGTGGTATCCTCAAACACAAATTGACAGGAGGGTGTGGTGTGGCAAGCTCATTGTGGGGGTAAATTGGAATAAGCTTAC
AGGGGGAAGAGTTGACAAAAGATAGGAAGAACCTTAAAAATATAGATGCCTTTTATGCAGTGATAAAATGTCTAGATATTTATACTGTGGTGAT
TATTAGGAATATGTGCAAAGATTGGCTATTAGGATGTTCATTACAGTGTTGTTTAATAATTATAAAAGGACAGAAAGCAATGTGGACTCAAAAA
TAGGAAAAGAATTTAAATAAATCCTAGTGTACCCGTTATACATGAAATTATGGAAATATGACCCTGAGCATGGAAATATGTACATGAGAATGTC
TAAAAGCTAGTTCATTTTGAAAAACAAAATAATGTCACCTCATATTATTTATAGTATATAAAGATGATTTTAAGAGTGGCAGTGTCTGGGATTA
TAGGTGATTGTATTTCTTCCCTTTTGCACATCTATGTTCTCTCATTTGTATTGTGTGGGGAGAAGTGACTTTTTTTATAAAAAGAAAAAGGTAT
ATGCATCCCAGCAGAGAAGCACTGGCTCCACCCAGTACCTGCCTCCTCATGCCACCCTCTCAAGCCAAAAGCCGGGGGAAGCCCAGGCACCTTG
ACCATGACCGCCCGAGACTCACACTTCTTCTTTCTCATCAGGGAAGGAAAGCCCCCCTGTAAGCGATACTCCAGATGAGGGCGATGAGCCCATG
CCGATCCCCGAGGACCTCTCCACCACCTCGGGAGGACAGCAAAGCTCCAAGAGTGACAGAGTCGTGGGTAAGTGGGTCACCAGCGGCCTCTGTG
CCTGTGAAACCTTTATCTCTTTGTATTTTTCCAAGACAGTCATGAAGGGATGCAAGTCATTTTATCCATTGTGTTCCCTCAACTGGCATATTAA
AGAAATATGGCACAAAGATCAGCAGGATGGGGGTCCTCTGGTGTGTGGGAGGATGGACACTCACAGGCCAGCATGGCCGTGAGAGCCACACACC
CCGCAAAATGTCCAAGTTGAGGAGCAATCCTGCCCAGGGACGCGTCTCTGTCACTGTCCTCTGTCCTCACTGCACTTGCAGGAATATCAAATGT
TATGGATTGTAGATCGTGAAAATTACACACTTACGTGTTTTGGCAACAGTGCTTTTCAGTGTTTGGGGTTAGAACAAGCCACATCTGGCCATTT
TATGTTATCCCTCTAATCTCTAGTTCTTAAATTTCAGATTTAACTGAAAATAGAAAGTTTCATAAATGGTAATTTTTAAATGTTAGAAAAAAAT
CAAAGCACACATTTACATACCTTTTCCATAAAAAAGTGTGTGTGAAACCATCTGCAACTCCACTGGTTAACTTACACATGCGACAGTTTGTGTT
TGGAAAACTTCACCCACCCTCTTTATTTCAAGAAACCAAAGACGGAGAGAAGTTCAGTGAATTAACCAAGGTGAAATGACAGAGTCAGAATTGA
ACTGAATTTCTGACTGAAAAACTAGTTCTCGCTCCATTATACCATGTTCTAATGAGCTAATGAGTCAACAGTTCCCTGGAATACCTGTTTTCTT
CTTTAAAAAGAGAAAGAGCTGTGATACTGAGAGCTACCTATTGGCATAAAAGAATATGAAGGACATACAGGTAAAATGAGGTAGACTGGATCAA
TGTGGGTCAGCTACATAAGCCCCCATAGCCAAGGAGGGAACATGTATGTAAAATTCTCAGGCTTATGAGAATCACAACTGTATGTCTATAGGAC
ATCCTAATGCAATAATGGGAAAATGTGATAACTGAATTTTATCTACATGTATAGAAAACATATTTTGGGCCGGGCATGGTGGCTCAAGCTTGTA
ATCCCAGCACTTTGGGAGGCCGAGGCTGGCGGATCACAAGGTCAGGAAATCGAGACCATCCTGGCTAACACGGTGAAACCCTGTCTCTACTAAA
AATACAAAAAATTAGCCGGGCGTAGTGGTGGGCGCCTGTAGTCCCAGCTACTCGGGAGGCTGAGGCAGGAGAATGGCGTGAACCCGGCAGGCGG
AGCTTGCAGTGAGCCGAGATTGCGCCACTGCACTCCAGCCTGGATGACAGAGCCAGACTCCATCTCAAAAAAAAAAAAAAAAAGAAAAAGTAAA
GAAAACATATTTTGTATGTTTTTTATGTGTCTTAGAAACCAAGTGGACTAGGTATAATGGTTCTTTTTTTTTTTAACTCAGCAGCACAGTTAAT
GGCACAAACAGATAAAGATGGTGGCTAAAGCCATTTGGTGTAGATGAATCCAGAAAACAAGTACATTTATGAATTATTTTACCCTATTGGAAAA
TGGCTACTATATGTGGAAAGTAGAGGGGAAGAGAAATGAACTTGTGAATTATCTACTCTATTCTACCAAAGAAAGATACATATATCATTTCAGG
GTTTTCCAAAGTTGGGATGCAAAGGATCATGTTTCATTTTAATTTATGTATTTATTTTTGAGATTGTCTTCTATGTATTGCAAGTGATACTGGT
TTTACCATGCACTGTACCACTACGAGGGTTCCTTTCAAAACACATTNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNTTAGTCCCTGAAGCACCCAGCGCCAAGGGTACTGTTCTTC
CTGCTTAACACAGGAGCAAACAGATGCTCACACCCTGCCCAAGAACACAGAGGCTCAGGGACTGAACCAGAAGAAATGATCCATTAACCATGTA
AATGAACAGAGAGCCTGGATGTCAGAGTGAGGAGGCCAGCTCGGGTCGCTGTGGAGAAAGAGATGGGCTCTCCCTGGAATCCCTGCAGCTGCCC
TTAGAGGCTGTGAGCCATCGTGGCTAGGGCATCTTTAGAGAACTGAAAGTCAGGCCGATTCCTGGAGAGTGAAGAATAGCACACGTGCCTTCAA
AACAGTGTCAGTATCCTGTCCCTTACGGACGTCATATTGTTGGTGGGTTTTCCTGGGTGGGAGATCACCCAGTATCTGGTTGGCATTACTGATG
GGTTCCCAGAGCTGTCTTTTGGTTCAAAGTCCTGCCCTCGGTCCCAGCTGCCCCCAGCACAGGTGTCACCTTGGAGTCAGGGGCGGGTTTGTCA
TAGCCCATGGGTCCTGGCCCTGCCCTTTATCATGTTTGACATGATTTTCAGTCTACTTTTCCTTCAGGATGTAATTTAGAAAATATGATTTCAG
AATTGTGAAATTTAAAAACCCTAGAAAACCTCTTTGCTTTGCTTGTAAATAGCAGTTGATGCAGCCCTGTGGACAGGCGGGAACAGCCATGTGA
CGTGGGCACTGAGATGATTTGGGAGATTGAATGCTCCTCAGTGGGGCTAGAGGCCACAGGAGTGATAGGTTACTGTAAAGAAAGCAAAAAAGAT
GCAAAAGCCTACATGACAAACCAGGCACGACACAAAGGAGAGGCTTGAGAAGAGGAAGTGAAGGAGGGAGAGGATTCCACAAGACCGAAGTGCC
CAAGGAGACATGGAGGGAAGAGAGGGCTCTTCACAGAGCTGAGTTTAGAGACCACCCTCCATGGTTCTGGAAGGAAACCAGCCAGCTTTCATAT
CTGGTGACAGGCTGTTCATTAATTTGTAGTGTACAGCATTAAATAAATGAAGTATGTAGAGAACATTAGGAAACAATATAAAAAGGTAGGGGAA
TTCGTGGATAAAGGGAATAACCTGGGTACCATATTTCATCACATTATTCCATGGGAATAAGTCATCAGTGCAAAGGACTGTAAGGAGTGCACAG
CAGGAAATGAAAAGACAGAAGCAGTGGGGCCGAGAGGAGGAAATGGAGTTGCTGAAATGCAGGGTGAGCAGCAGGGGCGGAGAGTGCCCTGTGG
GTAAGGCACACCTCAGAAGGGACAGGTCCAGGCCCTCTCCTCCCTGGCCTGGAGCTTCTGGCAAGGCTTTTCTTCTCTGAGCCTCACTTTCCTC
TTTTGAAGGTGGCAATAGTAACTGAACCTGCATCACAGAATGGCCTTGAGGGTTAGATGGAACATATATAGACATAATTTGTTGAAGACAGAGT
AGCAAAGACACAAAGTAAAAAGGACAGAAAAAAGAAATGCAACTTGTGCAAAATAGAACTTTCAAAGATTTACAAGAAGGGTAAATGAAGCTAT
CGTAGCTTCACTTGGGAAAAGGGAGAGAACAGATGACCAGCACCAAGGATAGAAGAGGGATTTCTTACTTTAAAAAAAATGGTCCCAAAGAATA
AACAAACTGAAAACAGCCAAAAGAGAAAGGAAGAATTTGTTAGAATAGGGCAAGACTAAAGGACAGAGTAAGGGTGCTGGCCGCCACCTGACTC
AAGTTCAAGTCGAGCTGCCACTGTTGAAATTTCAAGGAGAAATGGTGAGAAGCTAAAAGATGCAAAAAAAAAAAAAAAGTGGGAAATTATTTGC
TAATAATAGATTGCAAGAGTGTCTGCTTTAGAAAGTGACAAGGAACACTTTTGGAAAGCTGTGAGCTGTTATGACCACTGGCATCCTTCAGTTT
TAGACAAATCTAATCTCAACAAGGATAACAGACCATATTAATAAACAAATTGATGGGAATGGTGGAAATCAGTTCCCTATGAGGAGGGTGCAAA
GTTATACTGATTGGTGAGTCACTCCCAGAATAAACAAATAATGGTTTCACACCTCATTGCTTCATTCAGTGTGGAAGGAACATTCTAGGTTTGT
GTGTAAGTTTGGGGAGAAGGAGACAGAGCCAGAGGGCAGAGCAGCCCTGCAGCCCTCAGTTGGCCTCCATTTTCTTCCCGGTCTGAAGAAAATG
AAACAAAGGATTTAAAGTGTTGCTTGAAGAATTTAAGTTAGACTTAAAGGAGAGATGTGGTCTTTACTCTCCTTTTGGCAAGGAGAGTTTAGTC
ATCAGAATGGTCTATTTAGTGAGACTGTAGCTTGTTCTTCTGAAGTGGTCTTTTTAAAAACTAGAGAACTCTCACCTCTCTGAAGTGGTTGTGT
ATGAAACTGTTTAAAGCCGAGGATGCACTCAGTGACCTCTTCAGGCAACCCCAGATTTACTCAGTCAGCCATGGCACACATGCTAGAGAAGTAA
AAAAATGAATTGAGACTTGTTCTGAAAACTCTTGAGCAGGGGCTCTTAAAAGTTTTTTGTTTAGGAATTATGTCAGGGTTGGGGTCTGGAGAGC
AGATAAGTGTTTTGTGTGAGAAAAGTAAAAACTTAGTTATTTTCTTCCCGTATTCAGTAATCCTTGCATTTTTTTCCTCCTCCTTTATACATAC
ATTTTTGCCAGTTACTCACTTCTCTCTTGTACACATTTCCGTGTCCTAAATTAACCAAGAGTCCTCAGCCACTAGGATTCTATTGACAAGCCCA
CAGGTCTGGAGGTGTGAGTAAGGGCATTCCTCCCCCATGAAATCAGGCAGCAGGGGAGGCGTTTGGGTGGGTGGAACAGAGGAGCAAAAGACCA
CAGGACAGAGAAGAAAGATTGCAGGCTAGAAAACGGGATGGCCACCCCTGAGAAAGAGGACAAGCAAATTTAGGGCTTAGAGTAGAATTGAAGA
AAAAACCCAGGGCACAGGCACAGGAATCCTGTAGGACTGAACTGGCACCATGATTGATGTAAGTGGAGATGGGTTGGTTGAATCCGTGGAAGGC
CAAAGGATTGAGGCCCATATGGGCCTGGGACAGGGCTTACTAAATGACTTGCTACCTCGCTTGATAATCGGGACAACCTCAGGCAGTAGTTATT
CAGGCGGGAGGACACATGAGTTTGAATATAGGGAGATAAGGTATATTTCTATATGCCCTGGTACATTCCAGAATGTGCTTCTCTGGCTCTGTGC
ACTGGAGCAGCCAACTCCTGAAGTTGGGAGGGGATCGCCATGGTTTCATGAGTGGTTTATTTCAGCTTGCATCTCACAGTGTGGTGTTCAGAGG
CATAGGCTCTAGGCTCCCCTGGCAGCACTGAGAGATAGAAACTGAATCAGCACAACTTAGGTCCTTCATGTTGAATAGTTTGAACAGAATTTGA
TTTACCCTAGCCTAATTGAAAAAAAAAAAAGTAGAAAAGAAATCCTAGGTATTTTCCCTCTGGACTCCAGAACAAATGATTGAGTGTGTAAAAG
CATAAATTCTTTTTATTCATTCAATTCAAGTAAAAATTTCTTTACTCAGCCATGAGTTCTAAGACTGCAGTGGGAATTGTAAGAGGATTACCAT
ATTTTAAATTGGCTATTCTGCTGCCAAATCTCCCCTTCATTTTACAGTTTCAGGCAGAAGACCACCACGGTAGTCGGGGGAGGGGGGTGGGTAG
ACAAATCGTGGTAACAGTCCTCTACACTCTGGGAGGGAAGGAGCCGTGCTGGGGACAGCTTTCACCATGGAAAGAAAGCAGCCGCTCTGCACAT
TGGCCAGCAGGCTTTTGAGCACGGATGCGGAGCCTGCTTATCTAATCAGCAGCTAGAGGAAGTTGTTTTTATCTCCGAAATGCATCTCTTCTCA
ACCGATAAACCCCATTTTAGCTGGGGAGAAATATTATCTAGCAGCATCTGAGCATTAGCTGCAGCTGAACTCAAGCATGTTCAAATGTGAGACA
AGTCTCAAGCCGAGAACTGTTTGCCGAACTTCAAGTGGCTGGAGTACTGGTTTTCAAGTTTTCTTTTCTTTCTTCTTTGAGGAATAACTGAAAG
CATATTATTTGGATTCTTGTTCACCTTGCTTTTTTAGTTCCTTTCCCNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNTTAAAAGTGCTGTCAGAGTAGATAGATAACA
TTTTCTGTTGCCGGGTTTTTGGTCCCAGTTTTATTTTCTGCCTTGTTCCATCTGTGTTGAAAGAATAGTTAGCTCTGTGTATAAAACAACCAAC
TGACATTTCGTTAACTTTCCTTCTACATGGGGTTTCAGAAACAAATCGGGAAAAAAAGGCTGGGGGGTTGAGCTGTGGTTGCCAGTTGCCACCT
GTCCCCCTATCTGGTTAGGGCATTTTCTCATTTGGGAAGTGTAAATATAACCCAGAAAAAACAGCTTCCCCTCCTTTTATCGAAAAGTAGAAAG
CCTTAAACAGAGTACAGCCTTAACATTGCCCCGGAAAGTTTCCACAGAACCCGTTAGACTAAATGAAACCTCTTGCTCTGGGGGAGTGGGCCAT
GCCAGGAACCTGCCCTACCAAGCCCTCTCTTGCTGGCAAGGCAGGGCTGGGCCTGGGCTACTGTCTCCCACCAGATAAGCCCAGCTTTCTTCAA
GGCCCTAACAGGAACCTACATTTCACAAATTACCGCAGACATGCTCACAATTTTAAACTCTTTGCCTGTTAGGATCAGTGAGATAACAGAAACT
TATAGCGAACTCAGAAAGCATGCTCCGGTGAGCAGTGAGCCACCTAGAATCAGCCAACTGGTAATATGCGGAAAATCTGCCAGGTTGTTGGTAA
ACTTTTGGTAGCTTGAAGTCTACCGTGGTTGGGAGTATTTACACCACAGAAATCGGTGAACGCTGCAAATCAAGGCTTTTTCTTTTTGAGAGCT
GGTTTATCTGTCCACCACTAGCTTAAATACACTTTTAATGTCCTGACAGCTGAAATTTGACAGTGAAATTTGAAAGGAGGAATTCTTTTGGGTA
GGGTTGTGTGTGTGTGTGTGTGTGTGTGTGTGTGTGTGTGTGTGTGTGTGTTGTGGAGGAGGAGCTGCGAAAGGGTGAATCTATTGGGGAGGAC
TGTGGGGTACATATAATTTGTCCAACAGAAGAGTTGGTTCCAAATGACTCTTTGCTGTTGAAGTCACAAACTGGAAATGTTGGAGCAACTAGAC
TCTCCATATTCTAAACAAAACACATCTTTAGCATTTAGTTTGGTTGCTGGGGGAGTATGATTAGAAAAGGATTTGGCCAGGCATGGTGGCTTAC
GCCGTAATCCCAGCACTTTGGGAGGCCGAGGCAGGCAGATCATGAGGTCAGGAGTTTGAGACCAGCCTGGCCAATATGGTGAAACCCCATCTCT
ACTAAAATATGAAAAAAAAAATTAGCTGGGTGTGGTGGCGTGTGCCTGTAGTCCCAGCTACTGGGAAGGCTGAGGCAGAAGAATCGCTTGAATT
CGGGAGGCGGGCGTTGCAGTCAGCCAACATTGTGGCACTGTACTCCAGCCTCGGTGATAACTCCTTCTCAAAAAAAAAAAAAGAAAGAAAGAAA
GAAAAGGATTCTGTGTCTTATTTATGAGCTATTATGCTATTGCTGTTATTTGGGGCTGAATGATTTTTATTTGGGGCTGAATTATTTTTAAATG
TAAAGCAAGATGTCTAAATTACATTAATATGTTACTAAATTACTATGATTTTTCCAGAAAAATATTAGCCCATGCAATATTCTTTAATTCCTGT
GATTAAGTTGCTCTTCTGCAAAGAAATTTCTGTTGACTTCCTTAGATTAAATGCAGCCCTGAGATAGGTGTGCACCTCCAGCAAGTGGCCTGTG
CAGTTCTCCCTGTGCCTCCAGGGGTGGCTTCTTGAGGATACTTTCGCAGTAAGGGCTTAGCAATATCCGCATGTCTAAGGAGTCTATCAGAAAA
CTACCTCCACTGTGGAAGGGCTGGGGATTCTATTTTTGTGCTTTGAACCACCTGACACTTGCTGTAACTCCATTAACTGAAGAGCCGTTTGGTG
CCCTAATTTCAGGAGTGAGTTCAGCTGCAGGGATCTCAGAGAGACAGGTCTAGGGTGTGTCTCCGCAAGGCCTCCTGCCAGGAGAGTACTTCCC
CCACAGGGGATGTGGCTGCAGGCTGTCGATTTCCACTGAAATCACCAGGAGATGATTCTGGTGAAGCCCTGAACATCCGGTGTCACTCCTCACC
AGGCTGGGTGTGATCCATGGAAGGCCTTCAGCATGCCCCCCTGCTGCATGGCAGAGCCCAGTGGCACATGTGGCTGTTCTCTTTTTCAGAGTGA
AGGACTGTTACCCTAGAAGTGAAATCCTGCTGAAATGAGAAAAGTGCTGTTATTATATTTCTACAGGCAGCTATATGATCATTTTTATTATGAT
TCTTGGAACCTGGCAGAATTTTGACTGCTTTTGCAATATCTCTGTGCTCTCCAGTTGAACTTGGTGGCAATAAAATTAAGAATCTGCCAGTGCC
CCCCAAGCATTTCTACTCTGTCCATAATGTTTAGTGACAATTAACTATTACCAGGTGTAGCCACCCTTACTTAATCTTAGTGTTATGGTGCAAG
TAATCCTTAGTGAAATTATTGTTCTGTAGAGAATGCTGTGATCTAAGTGAAGTGACTTTCATCCTGAGATGACAATGGAGTTGTTTTTGTCACT
GTAAAGGCTGCATTTACTCGCAGTTTTAAAGCATTATTGCTACTTATATATGGTCTCAACACTGATTTTTATATTTGACTTTGTAGCTTTAAGT
GTAAAAATTTCTGTTAAGGTTTTATATGTGTTAGTTAATTTTTCCCATTTCAATAACCATGTTTTATTATTGATGGAGTCTCCTAAAAATATAC
CCTTAAAATCTGCTTGCCCTGTGCCTGCACACATATTTTAAATGTTGGGTATGGAGTCTTTTTTTTTTTTTTTTTTAGACAGAGTTTCACTCTG
CAGCCCACCCTGGAGTGCAGTGGTGTGATCTTGGCTCACTGCAGCCTCCACCTCTTGGGTTCAAGTGATTCTCCTGTCCCAGCCTCCCAAGCAG
CTGGGATTACACGCCCGCCACCATGCCCAGCTGATTTTTTGCATTTTTAATAGAGATGGGGTTTCACCACGTTGACCAGGCTGGTCTCGAACTC
CTGACTTCAGGTAATCCACCTGCCTCAGCCTCCCAAAGTGCTGGGATTACAGATGTGAGGCACCGTATCCAGGCTGTATGGAGTCTTTCTAATC
AGGACATAGGCAGACAGTCCTAGCCCACCTTTATGCCTTATGAGACGCAACAACGTTGAACAGTCATTGTTTGAGGGACAGAGGTTTTACAGAT
GGATGATAACTAGCATCTGTGGAACATTATTTGTGAAATATAGAAATCAGAAATTCCAGCGTAGCACTGTCCAAGGGGAACATAATTTGACCTG
CATATTTGCTGGTCCATTTTTAGTAGTCACATTAAAAAAGAAAAATGACACAGGTGAAATTAATTTGAATATATTTTCTTAATTCAGTATGCTT
AAATATTATTTAAGTATGTACTCAATATAAGCAATTGTTAATGAAATATTTTACTCTTTTTGAACTATGTGTTTGAAACCCCGGATGTATTTTT
TTTTTATCTTCACCACACATTTCAATTTGGCTTGGTCACATTTCAAGTGCTCAGGACTCACATGCAGCTAGGGGTTACCTATTGGACAGGCAGG
CAGATCTTGAGAGCTCCAAAGAACTGTGTGTCATTATATTGTGGAAAAAAAAAAAACTTTTTAGATTAACCCTTGTTGAATGTTAGCCAAAGAC
TAGTCATTTATCAACTCTCTAAAAGTAGTTTTTAAATCAATTTGCTTTTCAAAACATTTTCTCAGTGTTGTCACAAATGTATGTGAACATCTGC
TTTTTAAGTCTTTCATTTTTTGCCCACATTTGATGTAAAGAAAATCTAGGTCCAGTCCAATTTTAACATCAATTGGGTTTATTTTATGTAGCTG
TGTTTTAAGTACAGGTCTTGTACTTGTAGGCATTTTGGTTAAGTACAAAGTAACGAGAATACAACAACATGTGAACATGTCAAACAAATCAGTT
TTTCAAAGCTAAGTAGCTGAATTAAATGTAGAAAAACATGACATTTAATTTTTGTCTTCATCCCTTCATTGTATTCCCATGTACTCTATGGTAA
CTTTTTTAAGTTGGGAAGTCCTCTTTAAATTTTTGAAATGAGGTAGCTGCTTTATAGACATGAGGCAGGTTCCCAGAGAGCGCCCTTGAGGACA
GGCTGTAAGTAAACTAAGCTGCAAAGACAGACACCATCACAACTATGGAATTGTTTTGCAAAAATTGTGGAGAGTGATGCCCCTTGATACTTCC
TAAAAGATGCTTTTGCAACCTGATAAAGCAACCGAGGAGAGGTGGTGAAGGAAAGGCTTTTTTTTTTTTTTTTCAAAGAGAGAAAGAGAAGCAG
CAAGCTGCCTGCCTCCAGCCTGAGAAAGCAGTTCCACCTTCGCACCCATGTCCTCATAGCCCAAGCGGGGCCACCCTGGCATGTGAATCTGCTC
CGCAGTGTGCAGCCCAAACCCCAGCCTTCCTGTGAAAGGGACACCTCCTGCTGCTAACCGCTGCTCTTCCTGCAGTTCAGGAGACAGCGTGTGC
TCCTGTGGCGGCTGCATTTCCTGAGTACACCATGATCCAAAAAGGCACAGATTCGTCATGGAAGCTTTGATTGGGCCGTTACTGTCTTTTGACC
TTTCTTGTTTGCTTGCCCTTTTGTTTCTTTCCTCCTTTTTCCTTCCTTCCTGTCTTTCTTCTTCCTTCCTTCCTTTCTTCCTTTTTCTTTTTAA
TTTTCCTCCTTTCTCTTTCTCCTTTCTTTCCTTCCTTCTTTCCTCTTTTTTTCTTTCCTCCCTCCCTCCCTTTCTTTCTTNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNTGGTGTCAGCACTCCAGGAACACATGGGATCCCCGGGAGGTGGAA
ATTGAGCCGTCTTTGGAGAATCAGCTAATGAGACAGATGCATGTTAAATGTCTGTTGTGGCCCAGGCACTCTGCTAGGCAGAGGGGTGAACCAG
AAGAATGAGATTCATGGGGCCAAAGAATTTGCCTTCTGGTGTAAGAAAAGATGGAGGCAGCTTGGCAGAAACAAAAAAAAGGTAAAAGATAGAA
ATGAAATACAGAATAATCTATCTCCTCATCCCACAAGCAATTCTGCCTGGTTTGTGGCTCACGCCTGTAATCCCAGCACTTTGGGAGGCCGAGG
CAGGAGGATCACTTGAGGTCAGGAGTTCGAGACTAGCCTGCACCACAAGGTGAAACCTCATCTCTACTAAAAATGCAAAAAAATTTAGGGCCGG
GTGCAGTGGCAGTTCTGCCTGGTTTGAACATCTGGTTCAGTTAAAATACTTCATGAAAATATTTAGCTTCATGCACTGAAATCCATCACTGCTT
TTGTGGGATGCACTGACTGCAGTCTGACTGTTCTCCTAGTCGAAAGCGGAGCAATAGCTTCTGTCTGCATGTGCATGAAACAGATGGAAATTTA
GAAGAGATTCTAGTGCAGACAACTTCATCCCACCCCCGCTGCCCACAGAGCTAGCCATCTGCACGGTGTCTTTGAACAATGTTATGTTGCTGCT
GTGCACAGAGATCCCCAGTCTCATAAAACATACTGGATGGAAGGCTGTCGAGAGATCATCAGGCCCCAGTGCATAAGTTCTTCCTAAGCATCCT
TAACAGGTGATCACCTCGCCTATTGAGAACCCTCAGGAGGCAGCCCACTTACGGAAGCACTGATTGTTACAAAAGCCCTGAACTAAATCTGCTT
GTCTCTAAAGCCAGCCCTGCAGATGTCTGCAGTGTAACTACTTTCTCTTCTCTCAAGCAGTTCAGCCCTTCAAATCATTGACAACAGCTCTACA
AAGCCCTCTAAATAGCCCGTTCTCCTGGCTAAAGTGTGTTGGTTTCCTCAGCATATGATGAAATAAACACCATGATGCCTTTGCCTTGCTTTTT
CTTGCTGGTCTCACTCTTCCCTGGCATTCTTCAATGTGCCCAAGTCCTTCCTAGAACATTGCACCCAAAGGGCCTTCCTGGTCTTTCCAGGGAA
TGCAGAATGAAAAAGAAATCCCATCCCCTGCCTCGAAAGGAGCTCTTCTGTGTGTAAGTGTACCTTCCCCCTCAGGGCATAATGGGAAACAGTT
TGCCAAAGCTAACTGTGTAATTTCACATGACTCACTGCCGTTAGTCTGAGAAAGCATGGCATATTAAACTCATTTTCCATGCTTCCCAACTTTA
TTTACTATTCCATTTCTGAGGGGCAGAGAGCTGAGCAGGAATAACACCTTCCTTGCTCCTCCTAATGGGGCACATGTGCACATATAAAATTGTG
TTCGATCTTTGCAGCCACCACCCATGTTAACTGTCAACTGAGAACTCATGAGTCACTTTCTCAGGAACAGCCCTGAAGACATTATCTCCTGCAT
CTTGTATTTGTGCCTTTGATATTTGGAGGGAAGGGTAGGATCTGGCAATTATCGTTATCTCATTTGGTTCAGTGTATTTCATTTCAGCCTGTTA
AGATCTTTTGGAAATACTGTATGTGCTAGTTACAGGGACAGTCATAAATAAGACAGCATTCCTGTCCTTGAGATGCTATTTCCACAGCTCCATG
AGATGCCTAGTTAAAAACAGAGCCTTTTTTCGAAACAGCCTACTAATCTGTTAAAATAATAATGCAAAAAGATAAAGTTAGCATACCTGAGCTG
CAAGGGCTCCCTCCTGGTCTGATCTCTGATAAGGATCAGATCCTAGAGCCTCTGAGATCCCTGTCCTCCCTGTCTGCACAAGCACTCCCAGAAG
GAGAAACAGTTTACGGTGGTTCATCATGACTTTGAACCAAGCTTAAAGTCAAAGTCATACTCTTTAAACCATTGGAAACCAAGTTTTTGCGAGT
TGCCTAAAGTGGGCAAAAATCCCACAATGCACCTGAGCACAAGCAGGAAAACATGCCATTGTCTCCCCAGGAGCCCTCCTTGACTTCTACTTTA
CTTTATCCATTGACACTAGTCTTATAAGTAGCTTTGTCTGTCCAATTTTTAATTGAATTTCCTTTTTATTTTCGTGAGTGGAAATATCACTTTG
AAAGAAATCAGTCCTCTCCTGAAATCCAGAATTCCTGGAGCTCAGTTTACATGTTTGACGCGTGTCACATCATTCCACAAGTCACTCAAGGGCA
GGAGGATTACCATCGATACGGAAAGATTTCTTACAAAGCTTAAGTGAAAGGGAAACCAGGGAGAAAGTGTGCTTCGTGAGAATAGGTATGCGGA
CGGCTCTTTGTGACGCTCTGTGCAACCCTCGTGTTTGCATTGAACCAAGCTGTGCTGCAGATGGACACCCATCCCATTTCCCCGACTCATACCA
GGGAGGCCCACTTTGCAAGGTACACAGAGGGGACCACAGAGCAGGGGGCCATGCAGGGGACCAAGGGACATTTTAGTGTAACCAAAGTGTGAAG
TATGCCCTTTATTTCAAAAGAATAAAGAAAATCACAGGTTTTCCCGCTGATATGCCAGGGACATTTCCAAGAGAATTCCTTTTTTGAGAGAAAT
CTCCTTTGATATCCCATCAGTCAGCCATACTGCATAATTGTTAGATAGTGAAAGAAATTCATTTTTTAAGTTTGTCAGAAAAATAAATTCTTTG
AAGTCTTAAATTGTTGCCTCCGGACATGACATATCTGGCTCTTCCAGAATCCATGTTAGTCCTAGCTGAGGAAGAAGGAGAAGGAGAGGGGCGT
TTGTTGATTATTGATTTTGTAAGATGCCCCACACGTTGGCTATTAGCAGAATTCTCACTTCTAAAAGGAAAATGAGTGTGAGCTATGTTCAATG
AGAAACAGTTATTTTTGGGACATTCTTTGAGGTAAAACACCTCCTTAAGATGCTGCTTCCTTATTGCTATGGGACCAAGATTAGAGCAAGAACA
TAGTGGTTTTCAGACCCTGGACATCATCCACAGCCGCACCAGAGGCCCTGCCCACTTGAACAATGAGACAGGCCACCATTTTGTTTCGAGAATG
AGCAAAGTGAACCACCATGCCAGATATTGTTAAGTCAGCAACTCTTCTGAAGATGGACATAGTAAAAAATAAACAAACAAGGCAGCACTTGAGA
TGGTCATGGCAGAGCAATCTCAACAAGCGATTTGTTATTTTGCACAGTGATGTACCCACTCATTGAATAAAATGCACCAGAACCATGCACTACA
CATGCTAAGGAGAGTTGTCCTTCAACAAAGGAGATAGGCCCCACTGGCCGTGGGGCAGTTTATCTTATTTGGTCCTTGTCATGGGCAGGCATGG
CCCTTTCTATGCTTCACAGATGAGGAAGGTCCCTGGCACAGGTCCAGTCTCCAGCATGGCCTAGAGGTGGCAGGTGCTACTTAGCATCGCCAGC
CTCCTGCTTGGTCATGGGGTCAGCCAGTTTATAACACGAACGGAGGTTAATGAACTGATCTTCCCATCGCACAACTGGTATGAACCCACATCTT
CTGATTATAAATCTTTTGCTCTTTAACTCTTAGTCATTACCACTGTCTAGTGTAGGCCTGTGTGTTCATGCCCTTTGTTGCCACCAAAAGATCA
ACTATTAGCTGAATAACATACTGAGACATGTTGGTGTTGTGTTCTAAATAGCACTAGTAAACTGTTAGGGAAATCTGACTATATAGCTACTATC
CAGTCTAGTTTTTCTTGCGAAGTGTTTGTTGAGTGTGTAATGAGGAGTAAGGAAGGTGAATAAGAAATGGCCTGAGTCTTTATAAAATAAGCAA
GGAGAGAAAACAGTTTTGATGAGAAGCCCATGGAATTTTTAGAAGATAGGACGTGTATTATGTACCTATAAGAATGGGTAGGATTTTAGAAGAG
ATGGATGGGGAAAAGGAACAGGGAGTGGGAACAAAACGTGGACCAAGGAAGAGCAGGTTTAGCCATGGAAGCCTCACCGCCGGCTTTGGTTTAT
CGTGGGCCAAGCGGACAGACCCTGTGGCGAGGGCTGGCAGCAGGGAGGTCTTCCAGAAGTCTATCCTGCAGGCAGTAACAGCCACCCAGTCTAT
AAGCTGAGTGGGCATGGGGGTGATCGGGAATGGGTGGGGAGTTATTGGGGTAACTTACCCCAAAATGATAGCTAGCTGGAACCATTTATTCTAT
TGCATTTTATCAATAAATCTTATAGGAAGTACCATCCTAGTGAAAACCCTGTCACATTGAGGGCATTCGTGCTTATGTTTTAAAACATGTTATT
GGGTCTATGAAAAATAACGCTGAAACCTATGAGCACCCTCCATGCAAAGTTTCACTCAAGACTTTGGAAACAAGACAGTGTCTTACTCACTTTA
TAAATTCATTCAGAAAGCCGTAGGTTTGAAAATTCCAAACTTAGATGTAAGAAGCTCTGAGAAACACATGAAATCACCCCCACATCAGTAGAGA
TGTCTCAGCAGACATGGGAAGAGGGGCAGCAGGGTGTAGGGACGTCGGGCAGCCCCGGGGTGGCCCTTGCAGGCTGGGCTTGAATCCCATGGCC
ACCACCGTCCGCTGGGAGGCCTGGAGCCGGCTGCCCACTCTCTGACCAGCACATCTTCATGCTGTATCCTTGAAGGGACCCTGGTCTGACATCC
TGTGATGCAGACCTGAATCCAGCACCCACAGGGTCTGCACATTCCCTCTTTGAGGTGGAGCCCAGCTCCAGAGGCTGGTCCCTGACTCTGTTTC
TCAAGAAGCCTGTACAGATGTTCCCCTCACCACTGTTTCCAGTCACCTTTGGCTTTCACGGTGCAGATGCTAAGTTTGATTTTCAGAGCCCATC
TGGGAATTTAGTGAACTGAACAGGTAGCATTTCTGAACCCACCCATAACCCATGCCCTCCCCACTGATTTTGAAAGAGAGTTTGCTCCACGTGA
CTTTGCAGCTGGGTAGAGAATCTTGGGGCAGGATTCCGAGGCAGGCAGATGAGTGAGGATAAATTGGGTTCTGACGGCACGTTACACCAGTGGA
CTCTAACGACGACCTCACCTCGTGCACAGATAATTCTGCCTTGTGCTTAACCGTTAGAAATGTGTCACTGAAGTGTGAACATATTATGCTGTTA
CATTTCCCATCATTTCTGTTCTTTCATTCCCTCTCATTTGCATTGGTTACTCATAAATGTAGATCTTTGGTATGATTTGTACAACTGCCGGGTG
TCAATCTGTGAAAGAAATCGCAGAGCAAGCTGGGCTCTGGTAGCGCTTTATCCCTGCGTGCTGGCTTGCCCGGGTTCACTCAGAGGCAGTCTCA
CATTCAGCTGCGCTGGGGCCAAGGACCCAGGGAGCCAAGTGTGCTTCTGTTTTCTCTATTTAGCAATTTAAGACCTGCGTTTAAATACTAGCTA
TGCATTCTAGCAAAACAGGTTTATATTTTAACACAGTAACTCTTAATTGTTTAATTCACTTCGTGTGTTACCTCTCGGAATAAAATAGTGGAAG
CCAATTAACTATAGACTTCATTAGTTTGGATTTAAGATCACCAAAACATTTACCACATCTCAATTGTTCATTACATGCTCTCTCTTTTTTAATG
CAGTTTTTATAATATGGGGAGTGGGGGTGTGGATTTAACCATTATATTTTTATGATATTGGGAGTAGATTTAACCATTATGTAAACTGGATTTT
TTAATTTTAAAAAGTCACTTATCTTGATGTAAAATCATGTCTTAGTAACCTTGATAAACTAAGTTTTGCATGATTACACCTTAAGGTTAAAACA
TATTTCATTTCATCATTTCCCACAAGGGCCACTGAATTCACCCATTTCCTGTTTTTCATCTCAGAATGTTCTGTGTTTCCTCCATATCTACTTT
CCCGGCAGCAGGACCCTGGAAGCAGTCACACCAACCTCATTTACCCCACCTGAGATTTGTGTGCTTTGAACATAGTTGCAATCAAATCAACAAT
ATCTTTGCTCAGAAATGGATATGTGAAGTAAAATGTGCTGCCCTGTTCATGTATGAAATCATTCAGCTAGCTGGCCAGTGAGCCCTTCATTGCA
ACAAAGATTTTTCTACAGCCCCTGCACTATCTGGGGCTATGTCAGGCCTCACACTCCTCCTCACACGTTTGGAGGCTACCTTGGCCAGTATTAC
CTTAATCCAGCATTTAGGGGAAGGAGCATTTCAAGACTAAATTTTCTAAACTGCTCAAGCCTACCTCATTTTATTTCTTGTCTATTTTAACACT
TTTGGATGAGGACTCTTCCTAGAACCTACTAACAATTCCCCCCCGCCCCCATGCCAAGATTCTTTAAAGACTTTCTTGAAAACGCTTCAGTCTT
TTCTTCTTAGCTCAAAAGTACTATCCTAAATACTAGCTCTGGCATTACAGGGAGTTAATTTGTGGGCACACACAGTAAATTATAAACCCCTTAA
GCAGAGAAGATTGTATTATCAGTTTATTAGTTTTCTTATTCATTCCTTCAGCACACATTTCTGTTGCCTCCAATGCAGCAGAGAAATTGACTTC
TACAGTTTCCACAACAGTCCAGATTTCAACTGTGTAGTGCTCTTTCAGCCACAAACTACACTTGTGTTTCCCTGGCCATAGGTCCTGAACCTCA
CTTCTGAAAAGTCATTGTGCATAGAGAGCTAATACCTCTACCCTAAATGATCCTGGCTTTCAATTCTCTTATCTGCTTGGATAGTATTATCTGT
CTCTTCCTCTGCATTCTAATTTGCTACTTCTAATCTGCTGGGAATTACAATAAGAAAGAACCATTTAATCATTTTTACAACTGTGCCTAAAGAG
AGTGTGTCTAAGTGCCGAGAGAGTGTATGAGGGACTTGCCCATGAGTAAATCCATGAATTTTAGGTCAAGGGTTTTTTGCTTCTCTTTTGGTTG
ATTACCTCAGAGATCAGTTTTACTTTCTTTCTCATTCTTGACCTATCATCACTAGCTGATATGGATGATGTGTACAACTCTGAGTAAGAATAAT
NNNNNNNNNNNNNNNNNNNNCTGAGTAAGAATAATGTCAATGGGACGGGATGGGATTGGCTGGTGATTCTGTTGATCTTAAAGTTTATATATTT
TAAGTTTAGTGTTTCAGAATGAGACCAAAGCGGTGACATTTTCAACCTCTTCGGTCTCTCTTCAGTTTTTTCATTTTAAGTTTTTGTTGTGCTT
CTATCACTTAAAGGAACCCTCCAAGTTGAAATCAAATACTAATGACATTTTTATCTAATGTATAAATGTGTTTTTATTATTTATTAGGAAATTT
ATTTTACTTGGCCCTCAGCCATGACATATCATGGCATAATCACTATCCTAAATTTGTATATCTTATCCTTGCATAAGGTAAGACTTTCTATGAA
TTACACATATTTGTATTTTCCTCTCTTACATATTTTAAGAATTTTTTTATACTTTGTTTTCTGCAAATGAAATATTGCTCATAAGCCAGGTGAT
GGCTGTCCACGCCTCTTTTCCCCTGCTTACCCTTGCTTAGATCTTATGGTAGAATCTTTTCATAGAAGACACAGAAAGACATGAAAGAAAGAGC
TGGAGAAGCCTGACGGGCTGCCCAGTGGTCTGCTACGATGCTCTCCCACAGCCCAGGCACAGGAGGCAGGGAGAGCAATGGGGCCCTTCCCTTC
CACCAACATTCAGCAGAATTTGCAGCTCCATGTTTCCAAAGCTTCCAGGGCACTTGCATTTAGAGACAGAGAGCAAGCAAGCTGCCTTTCCTCT
TCCTCAGTTCTGCCAGCCACACTCTTCCCATGATGAGCAGTTTCAGCCAAAAGCTCCTTCCCCTGCCCTAACACCTCCTGCAAGGCCTGAGGTC
TGGAAGCCACCTGCGCCTGCTGGCCCCTCCTTTCTTGTTTCTGCAATGGATGTTGTGGCCCTGTGACGGACAAGAGAAAAAGAAGTTGCCCTCC
TCCCTCTATCCTCACCTCCTGCCATGCTGTCACCCTTATAAGAGAGAAGGGCTAACCATCCAGGCTAATCCTCCAGTGATGCAGGAGAGGACAT
CCTGGCCGGAAGAGTCAGACCTTCCAGGTGAGCTCAGGTGGGTCAGCCCCCGACGCTGTCAAGAGCCCAGGGGCCAGTAGATGCCACTTTTGCT
CCAGGAAGAATCTTCAACTGTGTCCTTTTTATTCAAGGGGCTCTCTTTTCAGCGAATCTCTAGATGTACTAGTCACAAACACTCCCATTTATTA
AAATGTATCCATGATCCCACAATCCTTTTACATATTTCTCTCCAGGAGTATCACATTTCTGAGGGCCCTGTGCCTGTTTTCTGCAGGAAGTTGC
TGTTGTCCCGGGTCCCCCTGCCCCCACCACCTCTGTTACAAGAAGCAGACCCTTCATGCCACACTGGGACCCAGGCAGGCCCCAAGCCAGGATG
CTGGGATCTTAGTAAAGGTTGGAATGATGTCAGAACATAGACGAGGCAGAATTCCCCCCATAGCATCATCCTGGAGGGCGCTGATTTGTGTGCT
CTGCCAGCTTCATCTGTGACTCAGGATTTAAAAGCCCCAGGTGTGGTGTCCCTTCTCTGCCTGCAAGGTGCGTCTTTAGCAGTTCTCCCTGGTG
TGGAAGGATCAGTGGTTCTTGCAGCCTAGGCACCCTCCACAGCAAGCCCAACACAGGTGCTGTGAGCAGCTGGTTCATGAACGGTGATCCTGGG
GAGAAGAGGAGGATGAAAATGGAAAACCAGTGCAAAGGTGTGTCATCCAGTTGGTTACTGCTGTGGGTGCCCGGGCTTCCATCCCACCAGAGCC
CCCACTAGCCAGGGCAGCCTGGCGGAGGTGAGCATCATTCTGCTTTCTGGCTGCACCTGTGTGGACAGAGCCCGTTCCAAAGCCTCCCAGGCAC
AGGGCACACATCAAGCAAGGGGCAGAGCTCGGGCGAGGGCTCTGGGTGCCCCCGTTATTGAACACAGGCCTGAAGGGAGCCTAAGAGCTGGCTG
GCAGGAGATACTACAAGGCAGAAAGACAGGGCAGCAGTCTTTGTACTGTACGTTTTTGTTTTTTAAGAGAGAACAAAAAGTCAAGGTTGAGAGA
TGAGAAATACTCTTGCCAAAAAGAGAACACCAAAATCCTGGAGTCACGGGTCTTCCATTACCCTCTCTAGCATTTCTGCTTGTCTTTTCCCTAG
TATTGGTGAAGAATTTTTAAAGTAACCAACCCATACCGTTGGTTACAGGCCCTGTGGTCACCAGGCTGTCTCCCCATGATGGGGATGGAGAGTG
GTTAGTCCAGAAACTTAGACCTCCCCTCCAGCTTGTTGAATGCCCTGAAGTTTATCTAGAAGGGAAGATACTCAAGCGTAGGATTTCAGTGATG
CATTTGGACAGCATCAGTACTATGCTTCAGCGTCAAAAACGTCACTTTGGGTAGGAACAATACATAATGCGTGGCAATGCCTTTTGTGACACCT
GTTCAGGAGATTCCCATAGGAAGCTTCTGAGGCAGAGTCCTCAGGTGAGGAGGGGGACAGGCCTGGGTCTTGGGGGAAGGTGGAGATGACCAGC
CTCATGCCTCTCTCCCCAGTGGCCTCAGTCTCCATCAGGCAAATCTTGAGAAGCCTCCTCCATTCTGCAGGCAAATGACTGAGATGTGTGAGCT
CTGCTTCCCAACAGGCGGAAATTCACATGGGGAAGGGCACCTGTGAATGGCCTTCTAGAACTATCACGAAGTTCTGGATTTAGTACCCTGTGAG
AGCAGATCCTCCTGGGTGCACTCGGATGATCCTGCCTAGCCAGCTGGATTGCAGAGTGGTCATCATTATCAGTGAGCTATTGAGGGGTTGAGAG
CAGTCAGTGTCATGTATAAGGATGGGTTGGGGTACCGGCCAACGCCCGCCTTGAGGCCTGGCCGCCGGATGGGAGGCGAGAGCACAAGAGAGTC
CTGGCTAGGTGTGCCCCCCTCTGCAGGGCCACACCATCCACTGGAAACCCTGTGCCCACTGGGCTGCGTGCTAAGAGGCTGTGGCCAATGCTCT
TGTCCAAATTTTACTGAATCTGCAGTCTCTCTTAATTCACTCCAAAGTTTAATGTGTTAGCTTGCGAATAAATAAATAAATAAATAAATAAATA
AATAAATAAATAAATGGAAAGAAACAGTCCTCAGAAAGTCCCAGTCAAATTTTAATTCCAACAGATATTCAGCAGTTTCCTCTAAGAACAATGA
GAGTTCTGGGGCTAGCCAGTGTTTCTCTTGGAAAATAAGCAAGAGGGAAAGCGGTGCATTCATTTAAAAACCTGCCCTAGGGAGGCAGCGTTCC
CTGTGAACTCCGAGCCCACAGTGAGCAGAGCAGCCTCGAGGCCTCCCGGCTCTCCTGCCCCTCCTGCCACATGCCTGTCAAAATCCTAGATAAG
GACTTTTTCTCAGCAACTTCCACGCTCCTTCAGTGGGGATGTCTTTGACTCAGAGCTCTGCCACTGGTTATCTCCACGAACAGAAAATGCCACA
GATGGGTTAATTCACTGTGTTCTTCTCATTTTCCCTCAGTTTCAGCCTTTTCTCTCCTTGCCTGTTTTCCTCGCTTAAAAAATGATGTGGGGGT
CCCTAAACGCATCTACCCCGATAGATTTATGTTTTCTTTTCCATTAGTCCACTTTGCGTCTCAGCCCTAAAATTAAGTTTTTGATTATAATGTA
AGGAAGTTTTACCATATTTTAACTCTGGCTTTTTAAATACAAAAGAAAAATAACAGAATCGGCTTCTAGAACTATCAGGAACTTCTGGATCTAG
TACCCTGTGGGGACAGATGGTCTGGAGCCAGCGCACTTGGATGATCTCTCTCAGCCATCTGGACCGTAGAGTGAACTTGTTACTGTCCCTCCAA
GGCTGACAACTATGACCTCATACTCTTCTAGCAGTTTAATTTAGACCCAAGAAAGGCTGTGTGTGTGTGAATTTGTCAGAGTGTGTGAGTGTGT
CTTTGTGCCTGTGTTTCTGTGTGTGTCTGTGTGTACATGTGTGTCTGTGGGGTGTGTCTGTGTGTGCACGGCTGTGTGTCTGCGCGTGTGCATG
GCTTTGTCTGTGTGTGTGCGCACGCACTCTGCTAAGCTACTCAATGCAATTCCTTACTCTTACTTCCCTTTCTGTCACTTCTCCATAATTCTTT
GTATTTCGGTTGGGCTGGTATCTCGCGGCGGCTTCCTCTTTTCCTGCATTCCTATATTTCATTATTTGCTCTTGTTCCTCTTCTAGGGCTTTTA
CAATATAGCCAGGAGGATGTGGAAACCCAGTTACAAGATGACACACAAGCACAGTGTCACAATCGCTGTGCTTGGGCCCTACCTCCTGGAGACC
AGGGTGGGACGGTGTCTCTGGATATGGAGGGAAGGGCGGAAGATCACGGGGTGGCAGAAGGCCCGACTGTCCGGTCTTCTGGAAGCTGGGGTCT
CGTGGTCGCTGGGCTGGTGGTGCTCTAAAACCTAGAGTAACCGAGAGCAGGATGACTGCACCCCTGACTCCCGACTCACCGGGCTTCGGAGGGC
CATGGGTGTGTGATTCCACTTGTGATCCTCCTACTTCCCCAGCCCACCCTGGTGGTCTCCCACTTCTCTCTCTCTCCCTGTGGAGTGTTATAAC
CGACTCCTTCTTCTAGGCATCTCCATCCCAACCCCAGACCAGTTCCCTTAAACCACCTCCCACCACAGGACCCCCACAAGCAGGGACCTCTCAC
AGTTCCTGTTGCCTGGGGACTTCATCTCAGCTTCCAAGGCCTCTGAAACCAGGCCCTGCATCCTGGGCATGACTGTCCCCATGATTCTCTGAGC
CAGCCANNNNNNNNNNNNNNNNNNNNNNNNNNNNNNNNNNNNNNCCACCCCCCCACCGCCAAGTCCACTGCTGACCCTGCTGTAGCCCCCCACC
TCCACTTGTTGAGATTCCCTCTTCCTGCTTCTCATTTTGAATGTCACCCTTCTGTGACTTCTCACAACCCCCCTGCCACCCCTCCCTCCAGTTG
GCATGGCTTAGTCTTCCTAAGAACATGTGCTGCTTCCCAGTACCCAGGGCTGCTGTGCACCGTGGCACGTGAGTTTCGGTGCATCTTCCTCCTG
CTGTTTATAGATTGCAACATCCCTGGACAGATCTTGGATTTTCCCTCTATAATTTCTCTGGAGACTCCCTCGGGGTTTGCACACACATCTGTGG
AATGCCCAAGTTGGAGATGGTCCTCCCTGATGTGTAGTGTGGGCTATTCAGAGGGCCTCATTTCATAAACTCATTAACAGCTAGGACGCGAAGA
CTTGGAGAAGTTAGCTCAGGACACACGGAGTGGGAGGGAGGCAACGAGTTAAGAAATGGGCTTTGGAGTGCCTCGAATTTGGCCTCCACTTTTC
TTTTCTACTGTCTTACCTGGTCTTAGGCAGGTCGGGTGACCATCTGGAGTCGGAGTTTCTCTCATTTGTAAAGCGGGGGTGATATTTTTCTCAC
AGGGCTGCTTCAAGGGTTAACAAAGTTAATGTCTAGGAAGGACATAGCATACCACCTTCACCTGGTTGCAAGCCCCTGGAGTCACATGGCAACA
GCTGTGGAACCAGGTAAATGACTTGACTTACATGTTACACGCTCTTAGTTGTTCGATTTGTAAATGGGAAGAGTGTGTGACCCAAATTAGTCGT
TTCCAGATATTTCCTGGTAAATGGTTGTTGAATCATAAAACTAGAAAGATGCGAAAGAAAGGGACGAACCCCCCTTACTTCGAAGAAGTGTTGT
TCATGTGAAGGACAGGACTTTTTGAGACACTCTCATCCTAAGAAACAGCTGATATCTACTAGGAAAAAACATGACATTTGAAGTTTCTTCCTAA
GAGATGTGAGGTTTTGACAGAAGTGCCAGGAAGCCAGAGGTGTGTGAGTAGTGGGCCAATCTCACGTGAAGGCTGTGGGACGGGCCACGAGAGG
GGCAGCCAGCATTTCCTCAGCGCCAGCTAATGGCGGGGACCTGGCACCTTTGCTCTGTGACTCTCCAGCTGTATGGTGACATGAGGCTGGCGTT
TTCTTTGGTTTCATACGTGAGGAAGCTGAGGCTTGGTGATGTCCTGTGCATGGCTCGGACTCCAGAGCTACTCAGAGGCAGAGGCCGTCTGTAA
ACACCTTCAGGTGGTTGGAGGCACGGTGCTGCAGCCAATGCTGATCACTCACAAGCCAAGGCTTTATTGTAAGGGCTAAATAAAATAACATATG
CCTAGCATTTATACAGCACGGGGCTTTTTAGGAAATAGTGAGACGACCATGGAGAGGTGGAGGAAGAGAGAAGGGAAAGAAGAAAAAGAAAAAA
AAAGCCTTAAAGAGTTTCTTAAGAGAACTATATATTACAAAGTCCTTGGAGTTTTTTTTTCCCTGGTTAGACATAAGTTTACATGAAACGTTAA
ACGGCTTAAAGTAGCCACATCTCCTTCCTTATGTCTTCTCACAGCCACTTGTGAGTTTCTTATCAAGATACAGAATTATGGCCAAGTGCGGTAG
CTCACACGTGTAATCCCAGCACTTTGAGAGACTGAGGTGGAAGGATCATGTGTGCTCAGGAGTTTCAGACCAACCTAGGCAATATGGTAAAACC
CTGTCTCTACAAAAAAATACAAAAATTAGCTGGGCATGGTGGCATATTCCAATAGTTCCAGGCACTCGGGAGGCTGAGACAGGAGGATTGCTTG
AGCCCAAGAGGTCAAGACTGCAGTGAGCTGTGATCGAGCCACTGCACTACAGCCTGGGCCACAGAGCAAGATCCTGTCTCAAAAAATAAAATAA
AATAAAATAAATTCTGCAACAAGTCACAATTCCTTGCTCAGAAATCTCTACAGGTGTGCTTCTTTGTTAAGAAATGGAGGAAACATGAATTTCA
TCCTGACTTCTGAGTCTTGTAGAGACCAGATCTGACTCCTGCAGCCTTCTGCCCACAGAGCTGGCAGGCGGGGGAATGTGCGCAGAGTGAGGAG
CAGCTGATCTGACATTCCGAACAGTGGAGACTGCTGCCTATTTGGCCGCACCACTGTCCTTCTGCATGGAAAAGTCACAGTAAATAATGGACTC
TCTACCTGTGAGTCGATTTTACTGAGCTGCCTTTTTAAATATATGTGTAGAAAGGGCGATCTCTCTGTGTCATTTGCACATGTACATACACATG
TACACACGTGCACACGTGGTCACTCATGTACACATGTGTGTGCACCAGTGCACCAGACCACATGCACCCACCCAATTCCACGCTCCTCAGCCCC
TTGCTTTCCTCTGCAGACAGCCAGTTCAGCTACAGTTATGCCAGATTGCCTGCACTCTGATCTTTACCATCACCAGACTCTCAGCTTGAGCTCA
TGCCTGAGAAATCCACTTTCTGGCAGGAAGGCAGAAGAAACTGCCAAGGCAGGACTGAAGACTTGACCTCCACCTGTAGTGGGGTCAGGTCACC
AGAGGCTGGCTGACTCCCCAGGATTTCTCAGCAGAGTATTACACAAATACCCCTTCCTTAAAGATTAGCAACCACTTAAAGACACAAAGTGTGC
AGGACTGTACCCATGGCCGAGAGCGGCGGGATTAAGAGGGCAACTGAGTTGTTCTTCCCATCCCTCCTCCTCTGTGCTGAGTCACCTTCTGCTT
GGAATTCTGACAGGGACCCGTTGGGCTTCTGGAAGGGAGGGACAGCAGAGGATCCACCCTCTGTGTGTCCTGGGGGAGATTACTTATCTCTGGC
CTCCCTGAAGCAGGGCCTGCGCTTCTGGAATCTTTGAGGCTGACACCCTGCCAGCCCTGGGGATGAGAGAGAATGGCCGTGTCTGTCTGCAGAG
CCTGAGGAGGAGCTGAGCACAGCCTCCGGAGTTCTCTTCAGTTGATCGCTTAGGTGGACAAAGGCCACAGAAATGGATTTAAACTCCTCAGCCC
TTTCTTTGCATGTTTGTTCTCATTTGAAGTCAGAAGTGATATGTCCTACACCTCAAGAACGTGTGAAATGCACATACAATAACCCCATTTCAGG
AAGCCAAGTCCAGCTTAACAGTCAAAACATTTCCTTCAGTCTTTAGTCCTTCACTTTGCCGAACTCCCTTTTACACCGGCAGCAACAGTTTAAC
CTGTTGCTTCTGTAAGAGTGTGCTACTGGGAAAACCACATCTAAAACACGTGTGCAGTTACATCAGCTAGAGCACATGCTAAACAGTTGATCAA
AGGCTCTTGCCTTGTGGCCCACGCTGCAGACACTCTGACGACTGCAGAGCTCCGCAGCCCCACGTCGTCCCTTCCGCACTGCCTGCTGTGCCTC
TCCTCTCCATGTGGCAGGGAACACAGCCAGTCATCACCATGTGGCTCTGCCCGGCGCTGCCCCAGCATGTCCTGACAGGGCCTAGATATGGAAA
GGTCGCTCTCCATGCACACACCCCAAGCCCCTCCTGCCCGCCGTGTGACCCACACTCTTATGGGCAGCCCAGTTATTTTGTAGCATTTCCCTTC
CTTATCATTTTGGCCCAGTGATCCAGCACAAATCTCCCTTATTAGAATAAAATTTGGAATGACAAAATTAAATTTCATTTTTCACTTATATTGA
GGACCTCACACTCTTCACCCCTCCCACGATCCCTGACAAGAGCCTTCCTATCTAATCATTGTTCCTCCAGCCCTCTTAGTTTTCTTCAGCCTTT
CTTGATTGCCTGAATGTCCCTTTCCCTTCTCCTTTTAAAGCATGAACCAAGCTTTCTTACCCCGTTCTCATTATCATTTTTGCATTTTCTTCTT
TGCATATGATTCTCCTTAAATTATAAAACTTGGGGGTAATTTCTAGAGGTGCCATCATAGTGCTTCTGTCTACTCAGTGTCTTTAGAATCAGCA
AATATCATTTTTACAAAAAAGTAGTATTTCTTCCAAAAAAGAGTAAGCAAGAAGGTTACAACACTGGGAAAATATCCCTAAGCCTGTTCTTCAA
CCTCTTCAATGTTTTCCCCTAAATTGTTATATGGAGATCCTGGACCCGGAAGTTGGCTGACATGAAACAGGCCTAGCAGGGCAGCTGAGGAAAT
GCTCCAACCTCAGGATCCACGAAGATTGCACATGGCACCAAAACAATCATTTAAAAGCTAATCCTGGCCACGCACAGTGCCTCACGCCTGTAAT
CCCAGCACTTTGGGAGGCCGAGGTGGGCTGATCACAAGCTCAAGAGATCGAGACCATCCTGACCAACATGGTGAAACCCCATCTCTACCAAAAG
TACAAATATTAGCTGGGCATGGTGGCGCGCGCCTGTAGTCCCAGCTACCCGGGAGGCTGAGGCAGGAGAATCACTTGAACCCGGGAGGAGGAGG
TTGCAGTGAGCCAAGATCATGCCACTGCACTCCAGCCTGGTGACAGAGCAAGACTCCATCTCAAAAAAGAAAAAAAAAGCTAATCCCATAAAAG
AACCATCATTTTGAACCTGCTGTTTCCTTTCTTGTACTCTTTAGAAGTACCCATTTCTCCCTTTCTAAGCCATAGGTGTATTAATTGGAGCTTT
TTCTATCTTAATTAGTTCACTGTGAACAATTAAAATTGTGTTAATAAAACAAAAACAAAAATAGGACTGGTGCCTAGTTGTACTACATGAAGAG
AGAAAGGGCCAGACATTGGTTTTTCCTAATCTTCTGTCAAGTTCTCCAAATCATCTGCTCTGGAAGGTAGCTCCAACAGCTGGGATTTGAAGTA
AAGCATAGTGACTTTGCCCATCACTGACATGTCCCATTTGAAGCAACCAAAACTGTCCCTCGACCTGACACTCATCCCTGAAACACCATGAGGG
TAAGTGAGGCGCTTCGGAAGGTCCACTCAACCCCATTGCCAGATAGAGTAAGTGTCTGCCAGGGGCATTTGGAGCTGAAGGGAAGACTGTACAG
ACTCACGGTTTGCAGGCACTGAAGGCGTTTTCCTGCCTTCTTTTTCACCTTCAGTCGACTTGCAAAGCACTAGCCCTATTGTTTTTCCCATCTG
GGAAACATGGTGAATGTTGGTTGGTTGTAGCTAATCCTATGGGTCTTGAGGTCTTTGTTGACAAGAAGGTAGATGTTATCTTTATCTGCGTGTG
GCTTTCTACTAAAACATGAGCTACAGGGCTCTCTTTTTTGTTTTAAAGCATTTTTCCATAAGGTTCACCCTTACTATTGCTTATCTGAATAATA
TTACCTGCTGAGAAGTTTATTCATTGCTCACCAGTTGTAGGGAGATTTTGACACAGGACTGGAGGATTTTTTTCTCATCGTAACAGTGCAGACC
CATGGAAAGCTTGGAAGCAGTTGTGACCGGATAAGAGCAGGTTGAGGATGATAATCTTAGGGCAATGAGCAGGTTGATTGAGAGGGGTGCCTGA
AAGCAAGGTCCCCATGATGCAAGCAAACAAACTCACATGCCAGACGGTGGACAGGAAACACAGGCAGGATGTGGTGCAGGCTGGGCTGCCTTGG
CTGGGCAAAGGCAGGGGCTTCATGGCCATGCTGGGATGTGACGTCGTTGGAAAACAATAAGAATGGAGACCTCTCCAAAGACCTCAGACATAAT
TTGGCCATGAGAGGCAAGGGTAGAGGCGAGCCCCCTGTGGGTTGGAATGTATGGAATCCAGATGAAGCACCATCAACATGCATGGGCTACATAG
GAGAGCTGGGTCTGGGGGAAAAGATGGGATTTGGGTTTGGGTGTACATGTTCCAGGCATGGAGGGAAGAATGGGCAGAATGACGAGGATCAAGC
CCTGGTGGAGCCCCCAGACCAGGGCCCTGGGCATATTTCCACTTGCCTTCTTCTGCAAACTTTTGTGTGCCTGTTGGGTGGAAGGTACTATGCT
GGGGCCATCCAGGATCCGGAGCAGTGTAAGACATCATGGCCTGCATGGTGTGACCCTTGACATCTGAAAAGAACGAGCTGGTGGAGTGGGAAGG
AGACAAGTGGCAGCAGAGTTGACATCATCAGGACTGACTGCAGGACAGGCGGTGAAACGAGCCACTGAGGAGTTACCCAGTGCCCTGGGGAGCT
TAGATGTGAGACTTACACCACCCAGCACTCCCCAGCTCTGTGTCCCTGGGTCCCACTGAACTTGATTGTATTTTAGTTGCCTCTTTTCTAAGAC
CGGAGCAACAATACCTTCCTTGAGTGTGCATGTGCAGTCCATAGATGGCAGCCAGTGTTCTCTTCCCATGAGAAGACTCTGAGCCTCCTCACTA
GAGGGTGGTCTAAAAACAAATGGATCCATCCACAGAAGGTCTGAAAGGTTTTCATAGAATCTTGACTTGGTAGCACTTCAGGATCTAACTCGTG
CTTTCCAGAAGTGTGGCCTGTGGTGTTCTGTCTGGAGTATTTGGTCCTCCCATCCACCTCCACCTTGCTGTCCTGTCCTGTGACTCTCACTAGC
TCAGTCACCGGGTCAGGAAGCCCTGCCATGAAATGCTAGATTTGGAGGCATTGCCAAGGTACCACAAACCATACTCAAACAAGGTCATAATAGA
CTAGTGATCTCACTGTGGCTAACAGGTTCATGTGATTTAAAGAATGACAGCATTTTTTCTAAATTTTATAAATTTTATAAATTTCTCCTAAATA
CAAATCTGAATGTGTTTACCTTTAGACAGATTTTCCCAGAAAATTGTCAGTGTTCTCGAATTTGAAAGTAGATCAGATCTCCTCCCTCTAGTTT
TGAAAATCTCAAGTAGGTTTAGCCTCTCCCCAGAGTGACAACGGCACAAGAAGTTTATAATTTTATGCACAGCTAAATGCAAACTGAAAAGTGT
CGTTTGTGGGTATTTCATTTTTCCCATAATGTTCATCAATTTGGCAATTCAAATAGGATTCAGAATCCAAGGTGTTCCCACCATTTTATATGAA
AATGTGCCTGAGAGTTTGGGGAATAGAAATTCTTACCAAATAAGTTAAATTTCCTCTTGATAAGAATATATTTTGTAAAATGTAGAAAACTGAG
AAAAAAAGTTTGACACCTGTTTTATCTGCATCCTTTTATTAAACAGTGGCCTGAATTTACTAAAGGACAGAGAACAGGGTTGAGAGGTAATCAC
TGAATAATAGCTTCGCTAATCCCACAGTGACTGCTTCTTATGACAAGCAAGACGGTCAACTTAGAGGCAGGACTGTCTTCAGGGGCAAAAGCAA
GTCAGCACCTACAAACCTCTCCAAGACACTGAGAAAAAGCAGGTTGTATCGCTCATTTTGCAGCTTGCCAGAAGCAATGTGGGAATATTGTCAG
ACTGCACAGACCTGGGGCCAGCTGGCTCCTAGAGTGCGAGGAGCTCTCAGTGACTGCTGTGCATACTCACCTGCTCTGTGACTTTGTTCAGTCC
CTACCCTGGATGTGTGGTGAAGGGTGCCAGGCTTAGCTGCTGGACCAGTGAGGTCCCTGAGGGGGCAGGGCTAAGAGGTGTAGCAGTCGCACTT
GACCAGTCATTGATTCACTGGTTGGGAATTTGTTCATTGAGGGGACACCATTACTCCCCATGCCTCACGTCCAGGTCAGTCTGTGTATATTTGC
ATTAACACACAAACACACTTATCTGTGTCCCTGTATGTGCAGCTGCAGTACCTTACACATCTTCCAGTGCCTCTCAAGCATCATAAAAACAGCT
TCCACAAAACCTCTACTACCCACCTGGCATCCACAGAGCTCCCAGTGATTGGCTCCCAAAAGATACTGAACTTTGGCATCGAACAGAGTGAGTT
TCCACATTTGCTCTTGTACATAATTACCTGTCCAGCTAGAATACTACACACTTCTTTTACTGAGCCCAGAAGTTTATGTCCCTGAACACGGATG
GTACAGGAACAAGGGTGAGCGCTTATCAATTCAGCATTACTCTGGAGACATGAAACAAACTAATAGGTACAGCTATATTTTGTTTTTTAAACAA
TTTTGTGAACCACATATTTGGGACAAAAATCAACAAGTAAATTTGAATTACACTAGGCAAAGGNNNNNNNNNNNNNNNNNNNNNNNNNNNNNNN
NNNNNNNNNNNNNNNNNNNNNNNNNNNNNNNNNNNNNNNNNNNNNNNNNNNNNNNNNACTTTCCAAAAGAAGACATACATGTGGCCCACAATCA
TATGAAATAAAGTTCATCATCATTGATCATTAGAGAAATGCAAATCAAAACCACAATGAGATACCATCTCACACCAGTCAGAATGGTTATTAAT
AAAAAGTCAAAGAATGACAGTTGCTGGTGAGGTTGTAGAGAAAAAGGAACGCTTATACACTGTTGAGGGGATTGTAAATTAGTTCAACCATGTG
GAAGACAGTGTGGCAATTCCTCAAAGACCTATATACCATTTGACCCAGCAATCCCATTACTGCATATATACCCAAAGGAATATGACTCATTCTA
CCGTAAGACACATGCACGCATTTGTTTGTTGCAGCACTATTCACGATAGCAAAAACATGGAATCAACCTACATGCCCATCAATGATAGACTGGA
TAAAGAAAATGTCGTACATATACCCCATCGAATACTATGCAGCCATAAGAAAAAGAATGAGATCATGCCCTTTGCAGGAACATGGATGGAGCTG
GAGGCCATTATCCTTAGCAAACTAACCCAGGAACAGAAAACCAAGTGCAGATGTTCTCACTTTTAAGTGTGAGCTGAATGAGGAGAATACATGG
GCACATAGACGCGCCTATCAGAGCGTGGAGGGTGGGAGAAGGAAGAGTATCAGAAAAAATAACAGGTAGTAGATTTAATACCGGATGACAAAAC
AATCTGTATAACAAACCCCCATGACATGAGTTTACCTATATAACAAACCCGCACATGTACTCCTGAACTTAAAAGTTAAATTAAAAAAAAATTA
ATGTCTAATAATATATTACAGTATTCTTCATATTCAATGGCAATGTGTGAAGTGGGAAGTGTCTTGACAGAATTCGGTGTTTCAAGGCTTACAC
TTTGATGCCCAAGACTGCACAAGGCTACATTTTCTACTGGTGAGACAAATTCCAGACGCATTCCATTCAGATCTAATCTCTTAGCTCCTTAATC
TTCAGGGTACTGGTAAACATGAAGACCTCCCCAGTGCTGTAGTCATCATGATATGTACAGCAGGTGGCTGAGCTCTGGATGTAGACTGCAGGGA
TATATTAGGAAGTTAATTCTCAAGGCAAGTCATCTTCAAGCACCATATCAGCATGATCAGCAATATAAGTAGTATCTCAGTGCTTTGTTGTTTA
GTCAGAGTTTTGTACTCTATCACCCATTGTAATGTTCCTATTTGCAAAAGGTAATACATACCCTTTAAAACATCTTTGCTTTTTCTCCCATTAT
CGAGATGCTAGCAGCTTCATAAAGCAGAATAACTAAGGGCAAACAGATTATATAAAGGGTTGGAGCTCAATGAAGACAACAAGAACAGCAAAGG
TTATTGTAAAACTGGCTGCTTGCAGGCCAACAAGCACATCCATATGGAGGCAATCAGTTTATGCTACCTCTGTCTGTTTCATCGGATTCATAAT
ATTGACTTTATCCATTAGATTTGGACTACCAGGGGAATAAAATAAGCAGATCCAGAGTAAGCATTTGCTAGGAAATAATTCAGCCAGTCACTTT
GAAAGCTGTTCAAGAAACAGCTTTCAAAGTGTCTCTCAAACTATGTTTGCCCATTATCCCAATAATTTATTTCCCAATAATTTCATGGGAAAAG
AAGGAAGTTCTGTGGTCAGATAAATCTCGAAAACACTGGTTTAAGCAAAGTTCAGTAGGTCTGCTTCCCTGCAGGTCACCTCAGAGTCTTTACT
CTGCTAACCTAGGAACTCATCCAACAAGTTTAATTTAACAGCTACACTGTGTACGTCACTTTAACAGTCACTGAGCTGTGACTCTTGGGGGAAA
GATTGTGCGTGTGTGTGTGTGTGTGTACACATGTGTGCACATGTGCAGAATCTACCAAATCTTAAGAGAAAGGAACATGCTGCCAAACTGTCCT
GTGAAAGAGAATAGAAACCTGAAGATTTGAGGCAGTGATAGCATTTATGAAAGCAGCACATAAGGACTAATCACCAAAAGGGGTAGCTCTTTTG
TTGGTTGGGGAAAACAGGAATTTTTCCCCCACCCAATGTGCTGCATTTTCTAATTTTCTATGAACACTTCCTAAGAAAAAGCTGAATGAAGAAC
ATTTGCGATGCAATCAGCTCATTAAGAAACACGCACTTTTGTGGAGATACGTGCTGTCCCAGGAGATGCTCTGCGAGGAGCCGAGTGTTTGGAC
TGGAGCTGCTGAATGGTTTCTCACAGTTCTAGAATGTTTGGGGCTGCACCCTCTAAGATGTTGAACCCATCAGTAATTGCTCCAAACCACTTTA
TGGGATATAATGCTGTGAGTTGACACCTGAGGGGATTGTGGTCCTGTTCATGAGTAATTACTTTTCTGTTGCCTATAGAAGGGCCAGCAATAGC
AGATGAGTAGCTGAACAGTGGTTTTGAGTAATAAAACGTTCTTTTTTAAAAAAAAGTAATGCTTTCTGTTAAACTCTGACTATACTCTCTCCTG
GTATCACAACCCAGCTTTCTTTTTGCCTTCTTTATTGCAGTTACATATGGGGCTGATGACTTTAGGGATTTCCATGCAATAATTCCCAAATCTT
TCTCTCGTAAGTATATCCCGTGCTTCTGCAAAACAAAAGCATGCCTTCATCTCCTATCATGTAAATATCGTACGTGCATGTTCCTTCATCAACC
CCCGAGATACATTAAATATTCACTGTTCTATTCGTTAGACACCTACCATATCATTTTTGGGTATTTATACCATAAAGTGCAAAACGAAGGTCTA
GGCAGTTGTGCGGTGTCCTGGGAGCATGGCAGTGGAGTAACAGTAAGGGTTGGAGTCACAGTAGCACTGATGGGATTGTTACTTCGCGGATGCT
GCTGGACAGCTTTGAGATTACTCCTGTAATCCTTTTTATTGACTGTGAGGATAAAATGGCCATATTTTCTGTCACCAACAAAGAACTGAACATG
GTTTCAAGATAATTTTTACAATTTTGTGGGACAGGAGAAGGAGATATGCTCTTCATCTGTGGTTCTGGAGTATTAATTTCAGAACTGAGAAGAG
AAAATTGCAGGCCTCCAGGCCGTTATTCTGTCGTCCATCCCTGCTGCACCCAACGTACTGTTCCTGCTTCGTGCCCGCAGGACCTTCTCTGATC
TGAGCTGTTAAACCAAAAACAGATGTGAATTTCATTTTTCTTTTTTAGATATTGAGCTGATAAATGATAAATATATTTGGGTAACATCCTCAAA
TGAGTACTTTAAAGAACAGAGAGTGCTTTTGAAAATGTATAGAGAGCTGAATATTTAAAGCCATACAAGTTGATAATCCCCTTAGGACCTGTAT
TAGTAAATCCTCTAATACTATAAAGCAACATCAGAAAACACGTCGCATTTGTTAACACTGTGACAATTATAATTATACCCTCTAATTTGAACAG
TGCCAGTAACAGTTAAAGACAGGTGTCTGACATCCTGGTTATCAAAAACTATCTGGTGTCTAAGCAATTTAACAGTATCAAAGTACATCTTTTT
GCTCACAAACTAGATTGGCTACTTTTCCAGCCTATAGAAGTTACAGAAATCTTTCTTTTACTGATATTCCTTTGATTCCACTTTAAAGCAATAG
CTTGATACCTACTTTTTGAGATGTATATGTATGTACATGTAAATGCTTATATATGTGTATTTGTTTACACGTCACCTTATATGAACATATAAAA
CATTATGGTATTGGACAGAGAAATGATATTGATTTGAAGAATCATATTGTTGAAGTATTTTGGGAAGTCAAATGCACTTGAGAGTAAGCTAATG
CATCCTAAAACATGTTTCTGTGAAGTCTGAGGAAGGTGTCAGCCAGAGCATATGTTGAACCAGACCCTTCAGTGAAGCCTTCAGTCTGTAAGAA
TCGCTGCATGTGAAGATGTGTTAATGTTATGATAGTTACAGTTTTTATAAAAGAGATGATATACACTTGGATATGCTTTCTGTCTATATTTATG
CAAATGTGTCCATAAGGTATTGGTGTCTCTCTTTCTCTCCCACTCTCCCCAGTGTTGGAATTGTGACTATCTTCTCACACAAGCGGCTACTTGG
TCTTGATGCCTTCCCCCGCAAAACAGCAACCAAACTGTTCTGGGCCAATATCACCACCTTGTGGTCATGATGAAGAATTGCCCCCTTTGCCCTC
AACACCTCTTTTCTTCTTGAAAATTAAAAACAACCCCTTTCACCCCCTCTACTGTCCTTATTCCAGTTTGTGTCCGTAGTTGCTGGAGGAAAGA
AAATGCCTATTGCTCTTTTTTTTATTCTCTTTATCTGTTTACTCTTCTGCCTTTCTTTTCTCTCCCTGACTTCATTCCATTTAAAGGCCTTAAA
TTGCAAATAACGAAAAAAGTATTTTCTAATAATATACCCTAGTGGGACGAAAACAACCTTCTACATTTTAAATGATATTAAGTTATAATAAATG
GTTCCATGAACTTTAAAACGCTTAAAAGTTTTATAAATCTCTTTTAGAGGCAACACAAAAATATCTATATATATATCTTCTCTTCACCTCAAAT
TTTCATTTAATTACCGTGATTCAAGATATATCTTAAATATTTCCATTTCATTTGATTTTTACTTGCTACTCACCAGTGTTGCAAGAATATTTCT
GTACCATTTTTCAAGTAAAATATTGAGCATTTCAAGCTTACATGGCCTAAACTCACACTAGAGTGACTACTTCGGACTATCTTTTCAATGGAAA
AATGCGTTCTGAGTACCAAACATTTACTTTTTCAGTAAATATGAAGACACATTCTAAAAAGGAAAGGGAAATGAAGAGGAAGCTTAATTCATTG
GTCAGCTGGGGACTAACAGTGTACATTATACCTATGCATTAAAAAATGTTTAATTATCCATCAGCACATACAGTGCCTGGCACAGATTCAGGCC
TCTTGGCTCCTCTTGGCATCTGAGTTCCTTGGATTCTCCAGATTCTTCAATCCAAACTTGTCTATCTACCAAAGTCCACCTTCCATACAGGGGT
GTGGGGTCTCTCTTAGTACTGTTGGGTGCCAATAACATAGAAGCCCTCCAAGATAGGTCAATCAGATTTTAATTTTTCCCAACTTTAAATAGCA
TCTGGGAAGGAAAGCAGCTTTTGACTAGGAGTTTGAAAACATCCACTCTTCATATTTATTGTCACATTTATTGAGTAATAAAATTGGGCTCTTG
TCCATTATCTCTCAGTCAATGAAAGAGCCCACGGTGGGCATGAGCCAAAGTGTTCCGGAGAAAGAGAGGACCAGTCTCCATTTCATGTTCTATA
TTTAATCAGTTCAATTCAGCACCTTACTGAGTTTGACATTTTTCTAAGTAATGGGGTGGGGGGTTACAAAATATTTATAACTATCTTTCCTATC
TTCAGGGAGATGTACCATTTCACTCAAGCAATACCACAGCTAAGCAGTTCCAGCTCCGAGTAACAGAGGAAGGAGTGAGCGATTTAGGAACTCA
CAGAAGGGCACGGCCAGGCAACTCGGGCCCTGGCAAATGTTCCATAGAGACAGCATCCATGGGGTTGAACCAAGCACAATGCCTGCAAATCGGG
CAGGCGGGTTCATGCAATTGATTCTAGGGCAGGTATGATCAGCACCATTTAAGGAGTGGCAGTTGTTAAGAAACTCAAGCGATTTCTCATATGG
CATTTTCAACAGAATTCAGCAGGAGCCTTGCATATCCCTTGGGATGGGTTCCGGGGTATTATGGAGCTGCCAGGAGGTGCTCAGCTTCAGTAAG
TGCCAGTCATTCCTTCCCAACCTCCTCCTTCATTAGTGGAAGTATCAGCTGGTGTCATTGCCTTCTTCAGGAGGATGGATTTCAAAATGAAGGG
CCAACAAAACTCACATTCTCGCAGAGCCTCCGTCACAACTGCATGTGTTCACTGCCATCAACAGGCCAACACCCACTTGTTCTTTTCTTCTAGG
TAAAGGAAAAGGTCTGTGGGACAAAGGCCCTGAACTCCTCACTCCTTGAATGGGAGGCATATTGGTGGAGGCTGTCACACAACTACAGGTGGCA
GGAACCTGCCATCAGGGTTCCCATCAGCCTCCAGGGCTTGCTTCTCTGCCCTGTGTGTGGACACCTTCCATCACTGGAAACCCCTCTCTCAGAA
GAACCACTTCTTGCCCTCATCTCCCACTTGCCTCTCCTCGGCACCTTCTCCACCCTCAGGGGCCAAGCTGAGGAAGTGTGCATCCTGTGCCACC
AACCTTGCTCGCAGGAATTAGTTTCAGTCCCCAGTGACTGACTCTTCCAAACCTGCACCAGCCTATGCTGCCCTTTTGAAAGGAGGCGTGCGTA
CAGCCTGGCTGGTACAGAAGATTCCAGATTTCAGTGACTACACGACGCTGTCACCCCTTCTGTTTTTCTTTTTTCTGTCCACATCTCTGATTGA
GCATACCTTCAGGGAAAACCCCAGAAACCTTTTTCACACTTACATGGGAAGCCAAGACACTCTCATCCTGCGCTTGTGTTTTTAAAATCTATAT
TTTAATTTCACAGGTCCTTACATTTATGACTAATAGATTTCAGATTTTGAGATACAATCTGTAGTTGCAACTTATGAAGATAGTTTCAAGCCCT
GATTTCTGTCATCTATGAAAACAGTAAGCAAGTTGCTTGTATTCTCCTCCTAGCTGGATAAGAGACCCCCGTTTTCATGGACAGCCTCCTTGGG
TGGACAGAAGTCTCCTGATCTTGTCTATGTAACCAGCACCCACTTTGCATTTTTCCGCAAAGAAAAGAGGAGCTTAACCCCATTTACGGGAAAT
CCACGCACTGCTTGAATCTTACGCGCCCCGTGGTGACGGTGTTGCCCACTCTGTTCCCTTCCCACCAAATCCTGCCAGCACCTGGCAGTCATCC
AGTCCTTGTCTTAGACTTCACCAACCTTTCCCGTCATAACATGGCTTAGAAGTTGTCCGAATAGTGCTAGAAGCTTTCAGCCCTGGAATGCTCA
GCATCTGCCATCTAACTTCCATTTAAAACGTTTTGAGCATTTGCTGATACAGGTTCCTCTGTCCTTTCCCCATTATTTTCAGTTCCTTGGCCTT
GCCCATAGTGATCTGTGCATATTGTCTGCATACTCTTTTTTAGAAACCTAGAGCATCGCAGTGTCCTTAATTTTCCTCTTCAAACTCTATATAT
TCCTGAGCTGATCATCCCTACTGCTGTTCCTACAGAACTGTGTGACTAATCTTCTATATATGTCCCTACATCTAGCCATCTAGGTGGAGATCTA
GGTACCTATCTCCCTAAAATTGTACTTGGTATCCACATTGACCTCAGGACAGTATGTGATAGGCTCTTGTGCCTTTTCGACTAGGAGTTTGAAA
ACATCCACTCTTCATATTTATTGTCACATTTGTTGAATAATAAAATTGGGCTCTTGTCCATTATGTCTCAGTCAGTGAAAGAGCCCAGGGTGGG
CCTGAGCCAGAGTGTTTGTGTCCCTAGTTCTCTTTTGGTTCTGTTACAGCTCTCAGTGACAGTGTCATTGAGGCTAACAAGGACAGAGTACTGC
TTTCAGCCACCATTTGTCCAATGAGTGGCTGATCTCCAGGCCTCTGGCTTTGAGAACATCTGTCATATCTAAGGCAGCATCCATTGTGGCCTTT
CCCCCATGCTTCTGTTTCCTTCCTGTCACATAGCTTTGCCTCCTCCTGCAAGCAGCCTGCTGTAGCAGAACCGGTGTTCCTGAAGCCAGAAACC
CAAAGGTCGTGTCCAATGCTTCCCTGCTGTTCTGCTCCCCACCTGCAAGCGCCCACACACTGATCAACAGCACACGCCATAGACCATGCCAAAG
AATCCCAGAAATACTCATTCTTAATGATCCAGAAGAACAAGTGATAGCTTCTGGCAAGTTCTAATACCCCATGTCTCCCCATGCGAAAGCAAGA
GTCAATTCTCTCTAGCTGGTTGCCTCTGAATTTATGAAGTCAAGCCGCGTAGGGGAACATGCCAAGAGGATTATCCCAATGCCTGTTCTGCGTG
CTGTCCTTTTTCTAGCCTCGGAAGCACTAGGCCTACACCTTCGCTATAGCCCTCGTCTTAGCGCCTGTATTTGAAAAACTTGCCACACCAATCT
TAATTTGCTCCATTGTCTTCTAATCTCATTTTAAAAACCACAAGGTAAATGATTAAAAATAATCTTAGATTAAGGAGTACACAGATCTTTGCAG
CCTCATTGTGTTTCCAGAGCGGGCTAGTGTAGACACTGGTGAACAAGGAGGGCCTAGAGAATTAGCTTTCTCCCAGAAAGATGCACAGCCTCTA
CCTGAGAGTACCAATTCCAGAGAACTCAATGGTCATTAAGCACCATTGCCTCGACAGCCAGCCAGCCTCACTTGCCTGCCTATCTCCTTTATTT
TCCAAGTATCTTGTCCCTGGCAGTGGCGAGAGGTTAGCAGGAGGCTGCTCAGATGCTCTCGGTCTCTGATCTTCAGGATCTGAAGGGGAGAGCA
TTTGAAGAATCCCCATTGCTGGATTTCTCAGGACAATCCTGCATAATGCCAGGATCTGATGGAGGAGACAGGCAGCTCTGTTAATCCTCTGGTG
CATCCTCACTTCTGTGGTCTCCAAGTCCACCATGTCCCAGTTAATTCATTTCATTATTCATCTGTAAGTCATTTCCCTAAAGAGCTAATAAGAA
AACACTGGCAGTACAATCAGCCCTCCATATCCATGGGTTCAGCCTCTGGAAATTCAACCCACTGTGGCTTGAAAATACAGTATTCAAGAGACAT
GGAACCTGTGGATATAGAACGCAGATTTTTCGTATCCATAGGTTCTATAGCGCCATTTTAGGGACTTGCACATCTACAGACCTTCAGGAGTCTC
AGAACTCATCCACTGCAGATACCAAGAGATGACTGTAAGTGGTTAGGAATTTGGATGCTGGGGCCAGGCTGCCTGCAGTTACCTTCCAGCTCTG
CCACTTGCCAGCTATGTGACCTTAGCAAGTTGTTCAACCTCTCTGTGCCTTGGCTTCTTCAACTGTAAAATAGGATAATGATAGCACTTCCCTT
ATGGAGTCCTTGTCAAGGTTAAATGGCAGAGTACAATTAATGTGCTGTGTGCCCAGCGTGTGCTATTCGCGTTACGTGAAACACTGTACTGGGA
TCACTGGGGGTGAGCCTCCATGTGCCATGCCCCAACCACTATCCTCAGCTAACTTCTTGTCTGTCAGTGTTAGGCTGGTGCTATAATAATTTCC
ATTTTCATGGATGAGGAAATCAAGGCACAGAGAAGTTACATCACTTGCCTAAGATCTCAGTGCTTTTAAATAGTGCAGCTAAGATTCCAGACCA
GGTATTTTTATTTCAGTGTCTGGACTGTAGATCTCTAAATCGAGAGGAACTCCCTGAATAAAATAAGCTTGGAGTGCTGTTAACTAAGTTGGTT
ATTTAAGACAGTTCTTCTCAGTCCTCAGTGTACATTAGAATCTTCTGGAGGGTATGTTAAAACAGACTGCTGGGCCCACCGAGAGTTCCTGAGC
CTGCAACTCTGGGTTGAGCCTGAGAGTCTCTGCTTCTACTAAGTTCTCAGGTGATGCTTATGCTGCTTCTCCCAAATTTTGAGAACCATTGATT
GAAAATGTTGATCAAAAATTATGTGGTCTAGGCTGAGCACAGTGGCTCACAACTGTAATCTCAACACTTTGCGAGGCCAAGTCAGGTGGATCAC
TGAGCTCAGGAGTTCGAGAGCACCCTGGTCAACATGACAAGACCCATCTCTACAAAAAACACCAAAAAAAAATGGCTGGATGTGGTGGTGCACA
CCTGTAGTCACAACTACTGGGGAGGCTGAGGTGGGAGGATCACTTGAGCCCGGGAGACAGAGCAAGACCCTGTCTCAAAAAAGAAACATTTAGT
CTTGATTGTCATCTATCTCATTGATCATTTTACTTGGCAAAATTTACCTACTTACTCTTATTAGTCTGTAAAAATGGTTATTAAAATGGTGGCT
TTCAGTGTAACTACAAATTCTCTTAGTCATTATAGTGTTGGATTCACCAATGTATCATCAGTCAGTGTTTCTGAAGTGTGTAANNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNCCCTTTGGAAAAAAAAGCTCTTCCAGATACCT
ACATTCATAAACTATCCCAATTAACCCTTCAGCAAGTCGAACAACTCTAAGAAAGGATACCTCTTCTTTAGAACACAGGGTTTGTTTTTATGTT
ATTTAAGATAAACAGGAATTCAAATGGTCATGTACCAAAGCAACACAAAGAACTTCCGGAAATCTCAAAGGGAACTGTGGTCAGAACTCTGAGC
ATTTTTATGTTTACTGAGTTTTGTCCCAAAGTTTATTAATGTTTACATGCCACAAGGAAAGGTAGCATCACAATAAGAGACGTTTTTCAGGCTT
GATAACCACTTATTAGGTATTTTGCCAAACAAGTTCACACATCCTAGAGAGCTGGATTGTGTGACCCAGAACCCACCCTCTACGGCAAGGTGCC
CATCTGATCGGTAGGGTGTAGCAGTAGGCCTCAGACCACTCCTGACCTGAACCTGCTTAAAGTGAGGGCCCAATTCTAAAGTGGGAACTATGTA
AATACCTTTCTAGTGCATTTTCAGATAATCGCCACTGGGCCTATGGATGCAGAGGGCTGGCAGATCTCCCTGTAACCCCAGGTGCATCCCGAGG
CCTGCCACCGAAGCCCACTCAAGGCTGAATGCACGGCGAGCTCAGGCTGCTCTCCCCTTGGTATTTGCTAAGAACTTCTGTTTAGTAGCTCTCC
ACACCTATTTGATTGTCTTTTTGCTGCTGTGTTGTTTTGTTGACTTTTTTTTTTGCAATGACACTGAGTCCCCTCCTGTATTGTTTCTTTCAGC
CAGTAATGTTAAAGTAGACACTCAGAGTGATGAAGAGAATGGGCGTGCCTGTGAAATGAATGGGGAAGAATGTGCGGAGGATTTACGAATGCTT
GATGCCTCGGGAGAGAAAATGAATGGCTCCCACAGGGACCAAGGCAGCTCGGCTTTGTCGGGAGTTGGAGGCATTCGACTTCCTAACGGAAAAC
TAAAGTGTGATATCTGTGGGATCATTTGCATCGGGCCCAATGTGCTCATGGTTCACAAAAGAAGCCACACTGGTAACGCCTGGCTCAGTTTTTC
CTTTAGTGGCCTGGAGAAGGTGCATGGGGTTTGAAGGAGGAAAGCATCCTGTCTTCCTTGTGTTCTGAGCATGTTTCTAATTGACTGGTAGCTC
AGTTGTTCCAAGCGATTGGTTCCAAGTGGTACCGAGTCATAGAGTCCTTGTTCTGGTACAGCCTTGTAAAGGACTTCTCAACACGTACCAATTC
CACCCTATAAATAAAACAAGGGAAAAGTGAACAGCATCACATGACAGGCTTGGCGAGGGCTGCTATTATAGTAACACATACTAAGTAGTCTCAG
CTGAGCCCTCAGGGTACCTGTGCTGAGTGGTCACCCTCCACAAAACAAAAAATCCTGATACACCAAAACTTACTCCTCAAAGTTTCCACTGAGA
AACCATGAGTAAAATGTGTGTTTAAATTGTATCCAAACTAACAGGGTTTGATGTTTAGAAACAATAATCAATGATGGAATAGCAGCAAAATCGA
GTTTTCAGAAAGACCTCAGATGAGCTTTCAAATGGCTTGCCCTCTAACAGGAAAGACTTTGAATCAGATGCTTCTATTGCCACTGGTTATCAGC
TCAAATTCCTAAAGAACTTCATCCCAAATACCCTGTCTTGCTGAAAGGTTTACTGGAAGTATAAGAGAATGTCATGTTCTGTGTCCAGAAAGGA
AGGAACACCGGCACCCTAGTGTCAGCCACTTGTGCTCAGGCTCACAGGATCCCCCCTCACCAGAGGCGTGGGAACACGGCGAGCCCCAGCTGGC
CGCGCTCTGCCACTGTTTCTAATAGCCGGTCACGTTGATGGAAGTGTCACAGAGTTGTCCAACAGAACTGTCCAGTCAGAAAACCACCACTCAT
GGTGCTGGAGTGTCTTAGAAGTAAAATATGAATAACACACACTTATTTAACTGTAGGCAGGAGGTTTCTTATGAATTGAAGAGAAACTTTTCTT
TGCGTCGGAAGCTGTTCTAAAGTTGGGTAAAACACAATAGATCCACCACCTCTAGCAGCCACTGATAGCTGAAACGTGAAACATAGAGACCCTA
AGCTATCACTGCCTCTGAGCTGGCATTGTTAGGTCATCATAAAGCTAGCGTCTCCCACTGCAAAACCCAAGAGGAAAAAAATAGTTGAAAATCC
TATTTTAAAGGCCTAGCAGATTCTATAAGATACCTTGGGAAAATGATGACGATGACTTGAAATCAGACCCTCGTATGCTGCTTCCGTGGGGGCG
AACAAAAATATGTTCATCAAAAATTAAGCAGAAATGCAGAAAATTTTGTGAGCCAAAAAAGCTGTGTTATCAGGAAATGCAGATATTTGTGGGG
TTGTAATTTTTTATATTTGAATCGGGCGGTTTTCAAAATGATCTATTCCATTTGTAGTGTATCTGAAAACCTATAAAAATAAGTTGATATCAAT
AGATATCCATCTTCCATAAAAATTCAACTTCTAAAATTAAGCAAACTTTGCTTTTTCTAATGGCCCCTTTATCCTCAAATTACCCACTGAAATA
GACGGATCACACTCAGCCCTAAGTGAAGCAAGCGTGCATGAGAGTAGTCCCAGCCTCGCCTTTGTAATGAGGTGGAAATTAACATGAAGGTAGG
CTACCCTGTGATAGACACTTAACAGGATACTCGGGGACCCATGGTAATACATCCCTGATAAGGAATAGACCTCACAAATGAACTACTTGCCTGT
TAATTCATTTAAAGCCTGACTGTACAGTGAAAATTCTCTTAAATAAATATTTGATAAGTGAATCAAATTCTGGCTTACTAAATTGCCAAAATAT
AATGACTGCCTGCCTTGATAAAAAGAATAATTACATTTAATGAATAAACCTGCCAAGTACAGATATGCCAGGTGGCACCTGCTGTTTGCTGCTC
ACTTCTCCCAACAAATAGCAGCTAGGTGCCACCTGCACACCAATAAGCTGAGTTTTTCACTTACGGAACAAATAACTTTCAGAAGTCAATTTTA
TAGTTTCTGCCTTGCCCTTTGTTAAAAAAATACACACACTTGAAGCAATGAACTCATGAATTGTTTTCATCATGCATTTCCACTTGCCTAAATA
TAAAGGGTCCCAGGTAGATACAGAAATACCTGGTTTGGCCAAACTTGGTTTGAATAACTAGACATGCTAGAAAAGGTTTTCATTTTCCTGGGAT
CTGAGTGGAATATGTTACAAAAGGCATGCTTTCTGAATTCTCTATGCTTAAAACATTTCTAGAGCAGTGCTTCTCAAACTTGAAAGAGCATGTA
AATCACCTGAGATCTCGTGAACATACAACTCTGCTTCAGACCTGGGGGGAGGCAGGAGAGCAAGCATTCCTAACAAGCTCCCAGCTTGTGGAGC
ACAGAGCCGCCTCAGGCAGCAGGGCAGTGCAGCTGAGCAGTGACAGTGAACAGGGCCATTCAGAGGGCTCTCCACCTGGGGCTTAGGTACGACG
GGAATCCCCACTGGACAGGCTAGGACTTGCACTCTGGCCATTGTTCCTCCTCCTGCCCATGGCTGAGTCAGCTTCTCAGTCCCTTCCAGGATAC
ACTGAGAGGATTCAGGGGCGGTCTCGCCTCTGCCCATATCCCCACGTTGGTGAAGTACCACTGGCGCCATTTTCTAATCAGCTCATCGGCACCA
GCACGTCACTTCCCCTGTTGTGCAGCTCAGTTTTATATTTCTGACTCCCGTCTAATAGTAACATCTTCCCCACCAATGTCTCAGGGTTATTGTG
AGAACCAGAGAGGAGAGAGGTAGGAAGGAAGCAGGAAATGCAGGGTGCAAATACACCACATTATTATTCTAAATAATGGGATTTTTAATAACAA
AGACCAAGAAGATTGTCTGTGCCTATCTAGTTCCCATCTGTAAGATAAAAAGGTGCAGTCCCTGAGCCTCCTAAAAACAGCACCCTAATGGCAC
TGCTCCTCCCAGGTATCATTGCAGTAGACCTACGTCAGTTCTTGCAGTTCAAACACATTTTTTCACTTTCTTCTGAAATGCCCTCAAATTCTAT
GACATAGTATCCATGAAGACTTCCCTTTTTACCCAGTGAAGTTACTAGAGGGGCTTCAAACCTTCTTACAGAGGGTTCCTAAAATGGGAGATCT
GTGGAGTCATTGGGATTAAAAATTAAATTTCAAATTCATGACATCAATGTCCTCATCCCAGTTAAATAAAAGTAAATACAACCTTGAAGAAATT
TGACCTACTCAGCTGCAATAGCTGTTGACCTGCAGAAGGCAAATGATGGACCCCGGCCACGCAAGAACACACATGGCGTGGGCTGCGTTCTTGT
GCCCCAGCTCCATGGACGTGTCTTCTTAGCTGTGGACCCCTCCAAAGGAAACTGCTTGAGTGCAGTGCTCTGTATTGTCACTCCTGGGAATGCT
CTCTTAACACCCCCATGCCTTGTGCTATGTGTTCATACCAGCATAGGCACTCAGAAGCAAAAAAGGTTTCCAGTGAAATTTGTTTGCAGGCAGG
TATTAAGACATCAGACACTGCACCATCCAAGTGAACCCCTGCCCGCCTGCCTGCCTGCCCTGCTCTGCTCTGCAGAAGGGCATGTGTCTGTAGC
AATCTGCCCTTTCTGTCTGCGATGGGCAGAGACCCTGGCTCTGCCTCCTGTTTTCCCTGTAGCCCTCCAGACCTGGGGAGAGACATGGGACTCC
AGCACCTGCCCAGCACGGAAGGGGGTCTTTCCCAGCTGTAAGTGTAGGGCTGCATTCACTAATTTAGGGTCATACCACAAACACTGTTGTGTCA
CCAGGATGTTGTCACATGCTTATTGTGAGCATTTTTGTGACATGAGCTTTCTCCTGAGAGGCCACCCTGTCCAAATGAGAGCTGTAGTTGGAGG
CACATCCGTCTCATGCTGCCTGGGTCTCCCTGCACCAGCTCACTCTACTTTTCCTTTTTATCCACTTGGAGTTAAATGCACACATTTATGCACA
AGTGCACAGAAGGCCCAGAACTGTTCCTAATAGATAATTAAATGATCTCTCTCCAGTGGAAACTCTCCTGCGCTACCTGGCTGCTAGCTGTTTT
AGCCTCTTCATCCAGTATTGATTTACATGCATCGGTTTGTAACTCAGACAGTATAGTTAGTTAGTTTGTTTCACCATCACCTTCCAACCTGCTT
ACGTTTAAACTCCTTCTCTCTGCTTAGCTAATACACATTTCTGTAAGGATTTGGAGAAATACCAGATACCTCCTGGTTAGTATCAGGACCTCTC
CATTGGCCTATAATTGAACATCACTGTAAATTAGTTTCAGCTTTTATAGTAATGCATCTAACCCGCCAGCTTAAAAAAAAAGTGATGATAATCA
TGAAATTCAAGTGTCAATATAGACTTTTAAATCTGGTGTGGATGTTAATAGTGCCAGATATGCTTCTGTGTAATCATTTTTGAGAAGTGCTGTC
TTCTTTACCATAATCACATTAATCAATCGGGAAATGGTTAAATAGACATTTTAATACATTGTTTTTTCCTGAGAAAAAAAGTGACTCCAGGAAA
TATGTTGTTACAATATCTCAACACCAGGAATTGTGGTCACATAGCATTCTTCTTCTAAATAAAGCATTTGTTTTACAAAAGAGTTTCATGTTTA
CTTAAACGTGACAAATGTATCACTCTTCGAAGTGAGAGTTAAATGAAATGTTCCCTGATTTAGATTCCAAGACCTGTGACTACTCAGTAGGACA
ATAGCATATTTCTTTTACTCAGATACCTCTTTATCCCTTTGTCAGCTCCAGAATCCAGACACAGGTGCTGCCACACAGAGGAGAGCGTCAGGTG
TTTTCTTAGTCACAAATCCAGCAGTCTCACCCCTGAAAGAATTCCTTCATAACAACTAAATTGCACCTCTTAGAATACCTGTACTTAAAATATA
ATTTGTCTTCTATGGATATAGACAAATAATAGCTTAAATGTTTTAGGATTTGTAAAGAAAGAAAAAGCTAGAAATCTCAATGCAACTCTGACTT
GTATGTCCTGCCATAGGAAGGTTTGGGACTTTTCTTTGGAAGATGGGAGGCTTCCTTCACAGTTTCTTAACTTCTCATTTTATTCAATCTTTCT
CCATCTCTCTCTCTCTTTCTCTCATCCTCACGCTCTCTGTCTGTCTCTCTCCTGATTTGTCTGCTTAGGGACAGACAGGTAATGGAGAAGAAAT
TGAAAGGGAAAATACAGGGAAAATGTAAATGAAAAGAAGTTATTATAACTGTTATAATGAAGTGCCTTTCATTTAAATATAAGAATGTAACGCT
GAAATGAACTTGTGATCCTGAAATGCATTTTTAATGAGTTTCCTTTTTATTTTGCTGCTTCAGTGGCATATTTTAAAGACCCTTTGAAAAAAGC
CACATTAAAAAACCCCACCAAATGCCACAACACGCAAAATTGGATATTGGTTTATTCCAAAACTGCTGATCAGGAAGAGTGGCTCTTCATCACA
AAATGTTGCAGTCACTTTATTTAAATGAGTTTGAATTTGCATTGTGATGTGCCATTCTGATTTAGGGAAAAAAAATTAGATTTTCAGATCAAAT
TGACCCAGCCAGTGAAGCGTTAAGGAGCTGGCAGGTTTAGTCTGAAAGCCTCATGTTCTATCAAACCTGCAGCCGGTGGAAGTCACCAGGCCCC
CGTGGGAAACAACTTTCTCGTAGCATCGTCCTCATGTCCCCACGCTGACTTTAGTTCTCACCACCTCTCCTCTCTCCGTCCCACGAGAACGGCC
CTTCCAGTGCAATCAGTGCGGGGCCTCATTCACCCAGAAGGGCAACCTGCTCCGGCACATCAAGCTGCATTCCGGGGAGAAGCCCTTCAAATGC
CACCTCTGCAACTACGCCTGCCGCCGGAGGGACGCCCTCACTGGCCACCTGAGGACGCACTCCGGTAGGTCCCCTGGATGCAGTCCGGGGCTGT
CTGGGTGTCCCGCGATTCCTCCACTCTGCCCGCCTGGGTCCCGGATTGTGTCCTTGCTGGCTAACCCTGAGTCCCTCCCAGCTCGCAGTCCTGC
ATCGGGTGTGAGCTGTTGCTTCTTTGACATTGCCCCATCCCCCCTCCCCATATTCTCCTTTTCCCACTGCACCAGGGAGATTGCGCGCAGGACC
CCCATGCACATACACACACAGAAGTCTCACCTTAGCTAGCATGTTTCAGAACAGGCCTCCTCATCCCGTTTGTCAGGCTGGTATGCTTTCCCTA
AAGGATTTTGCGGGAAATGTTTTCACCAAGTGTACTGCTAAGACCCAAAACGTTTTCAAGTACAATTCTTTCTGTTGTTAAGTCCCGCTTTGGA
GTCTTTTACACAGGTCTAATGGATACCTTTTTTGCAGAGCTGGTAGAGAAGGGTGATTTAGGGCGCACCCACCCAGACTGAGCCCCGTGTGGCT
CTCACACCAAAAACCAGCCAAGGCCACAGTTACAGAGGCCAGTCTGGGGCTGTTACCAGATTTTAGACAGCAGCCTTTCTCTTTGAATTAGACA
GTTAAAGTACAACCCACATAATCTGGAGTCTTGACAAGATCATCATAGGCATAAACGCTCTATCATTCTCAAAACAGTCTCAGCCTGCAAAGTT
CAAATCCACTAAAGTTTGGTTAGATCCTCTGCCTCCTGAGAAATGGTCCTCGGTGTTTCATTATCCAGCAGTCCCATAATTCTACAGGGCAGAG
GAAGAGAGGGCTCTTGGCCGCCCTGTCATGGATCATGTTTGCCTACAGTGTGGTCTATACAACATGACATGGCACAGGTCTCCTTCATACCGTC
CAGTTGGGGATATTTGCTGTAGCATACTGCATGAGACTTCGGAGGCGAAAGGTTGATGGCTTTTGTCCTTCCCCTCAAGGAGCTTCCTGCCCCA
GCCAGCCACCAGCCAGGGCCCTTCTCCAAGCAGCAGCCTCTCTAAGCCGGTGCCCTGGGGGATGGCAATGCCTCAGACCAGCTACTCCTCACCC
ACCCTGGGTAGCAGGATAAGGAGGAGCCTCCCTCAGGGAGGCAGACGTGTGTTCTTTGTGAAATATCTGCAGCGGCCCAGGCCATCTCTTCCCA
AATGTGATGCGTGTATTTGATGGTTGAGGGTTTTAGAGGCTGCTCATTGTCTCCATCTCTTTATCACACATTTACTGAGCACCCCTGGGTGCCC
ATTCTGCACACAAGACCTCAGGTGCACACAAGGAAAGCACCAATGTGTTACAGGAGGCATGACAGAGTGTCTGAGGGGACAGTGGGAGCATAAG
GAAGGGGACAGTGGGTCCTGGGGTGGAAAGTCACGAGAGACTTCCCATAGAAGGGAGGGGCTTCTGAAGTACATGCTGGAAGGCGACTGTCTGA
GGTTTGCTTTAGAGACATTCTAAACTAGTCTGTGAGTCGGCAGTAATCCCAGACAGGGGTGCCGCACCCATGTTGGCAAGAACTCGGTCTCACT
CCTGGCAGGTGGTCCCTGCAGTAGTTCACCCAGTTGGCTGGAGACAAAAAGGAGAGGAGTCACAGGGGCTGGTGCATCACCTCCTCTCCATCCT
GACCTCCTTCCTGCCGGTGCACATGGAGAAGGATCCCCACATGCTCGCCATCAGAAAATTGTCATCATTTGGGTGATTTGACTTCCTAAAAACT
TCCAAGAAACGAGCTATACCAACCTCAGGAGTTGCTTGCCCGAGAGGGCGGGCAGATCAGCAGGGGGGCCATGGAGTGGGACACTTGCCTTGTT
ACAGAGGGACAGGGAGAACAGGTGGTTCCCCTGAGATAGGAAGACAGTAGCATGACGGTGAGCATCTTTAAATGTTCACATGGGTGATGAGCAT
GTATGGTGGTCCCACCTGGAGACAAAGGTGGTACCTGGGCCTGATAAGCAAGCCCCATCCAGGCCTCAGCTAAGAGAACTCTGCAGTCTGTCTG
CCATGCTAACCAGTGGTCTAGGAAGAAGCTTCACATCTGTTGTTTCTAAGGAGCCATGTAGACATAGACACAGTTGGCAGCAATATTGCCTTGG
GTTAGTGGGAACCACCACTTCCCATGTCAGTGGAAAACACAGACACTCATTCTTACGTTGACACCAACCATAGGTCATCATCACTCCTAGATTA
CTCTGATTGCAATGGAATCTCTCATGCCCATCAGTCCTTAAAAGCAGAGCTGCTCTCTTTCCTTTCTTGTTGTCGACTTAGGAACATGTAGTGG
AGTATTTTATGTTATTCTGGCTCCAGGCCTATGTATTTCATTTGAGGCATGATGGTTTCCTATTCTAAGTACCACTAAGTATTGAGTAGTTATA
AAATGGTGGTAGACTTTCCACTGTGTATTTCTATTCTGGATCCACGGATGAGCAAGTGGAGAAAACCCATCATGGATGCAGGGCTGCTGGGGAT
CTGTCTGTCCGAAAATGCTATAGAGACACACACTGCTCTTGGGGAAAAAAACAGTGATGATTTCTTCAAAATGATTTCACTGAAGTGAAATAAG
GACTTTGGCCTTGCTCTGGCAAATTCAAATGTGGCCACACTCTACTCCATTGTGGAATGGTGACTCCCCTGTCCCATGAAAAACCAGGATCAAC
ATCACAGCTTTCTCTAGCAACTGGTGGTGGCCTGAGGTCTTATGAACTAGCATGTTAGCAGGGATAGAGTTAGGGAATTTGTCCGATGCTGACA
GCAAGAGGGTGCTGGGGGAGCTGGAGGGGTGGAGAAGTGATCTCCCTTCCTGCCTCATGGCTTCCTTTGGAAGTTGCAAGCTTAGAATTTCTTT
CCCAAAGAATATTGGACTATGCTTCAAGAGACACTTCAGTTCAGTTGCTTACAGTGAACATACTTCTTATGTTCCAGAGTTAAACTCAATCATA
TTTTCAGAAAAATAGATATTCAGACATCATGTCATATTTACACGAAGCTATTTGATCATGGAATTTACTGAAGTGACAGCTTTTTCAGGAAAGG
GTATCGAGTGGTTATCTATATCCTATTAATTTCTAAAAGCAATGCTCAATAAAATGTGCAATTTTAGGCAATATTCTGTGTTTCAACATTATTT
CCTTATGTTGGGGGTACATCACTTTATATAGATAAAGAAAGATCCTTTTCATTCTCTATTAAATGGTCCAGAAGTCAAATTTTGGCTTTCATGT
TGGTCTTCTTACTGTAGCTTCTGTTCACCTAAGAAAGAGATTTAACCAACATAAGCTTTGGCTAAACCATTGTACCAAACATTTCTATTTGGAT
GCTCTTAACTTTTATACAATACTTTGAAATGTGTTCAATAAAACAACATGACAAAGAAGTAGAAGATTCTGCTATTAGGTTCGAAATTGATATT
CCTTACATAAGTGATTAAAGATACTTTATTCACTTTATCCCTTAAAATGGTATCTACTGAAGTCCTTAAACTTAGGCATGATATTAATTCCTGG
CTAGTCTTTCTTTTTCCTAAAATATCACTCAATAAAATCCAAGAAGGACTAAAACTCATTATATAGAACTGCACTGCCCAATACAGCAGCCACT
AGCCACATGCAGTATTTTTAATTATAATAAGTTAAAATTAAACAAAATTAAAACTTCAGCCCTCAGTGGCACTAGCCACACTTTTTGTGTCCAA
CAGCTACATATGGCTGGTGGCTGCCATATTGGACACCACTAATACAGAGCATTTCTATCATCATAGCAAGTTCTGTTGAACAAGGCTGATCTGG
AAGCTACACCATCCTAAAGCTCTTTGGCAAGAAAGCAGAGCTTTTCCATAAGCTCGGTTTATGAACAATATATTTTGTATTTTCTATGTATATG
TATATGAGAATACGTATACATAGAAATATATGAAAATACATATACATATAAATATACATATACATAAAATAGAAATACATATACATAGAAAATA
CAAAATATTTTGTATTACATATACATATGCATGGAAATACAAAATATTTTGTATTATATAATGCAAATATAAGATATATTTATATTATGTAAGA
AAATACATTTGTGCAATAAAACGCTTATTTCATTCAAGCATATTTGTCCAGCATGGTTTCTTAGAGACTGAAGTTACCCAGTTTAGCACCTGGA
ACTGAATTCACTCTGGCTGTGTTAGATGTAGCACCAGCGTCATGACAGATCCAGCCCACAGCATAGACTGAGGCCTACTCTGGAGGCAAATCTG
CAGACAATGTGGCCAACACATGGGCCTTCCTCACATCTCTGTCCAGTCGCTGGCATCCACGTTATAACACAGCAGTGGCTCGTTTTCTCCAGTT
AAAGGCTAGGCTTCCCTGATTGAAGCAGGAGAGAATTTGGTAGTCAGTAAAGTAGACAAACTAAGACAAAACAAGAGTCGCATGAGTTCTTGGT
ACAATTAAAGAAACCCTTGGCCACCAACGTTTTTAAATTCAGGAATTTCACCAAGTCCGTAACAGTTTTAGCTCTCAAGGGTAGAATTTTTTTT
TTAACTTTTTTGGTAATAATTGTATTGCATGCATTCCCCTTACACAGAAGGCTGGCATTTAATTGGGGTCTTGAACTCAATTGTGTTTTCTGCA
GTTGGTAAACCTCACAAATGTCCATATTCTGGCCGAAGCTATAAACAGCCAACCTCTTTAGAGGAACATAAAGAGCGCTCCCACAACTACTTGG
AAAGCATGGGCCTTCCGGGCACACTGTACCCAGGTAAGCGCTGCTGCTCGGAGGCCAGCCTGGTGGGCTCTCCCCCCAGCACGGTGGGGAAGGA
GGGCGCTCTGCATGCAGCCTTAGGAGCAGAGCCTTGGCCCTGCTTCCTGCCGGGGCTAGGAGGGACGGAAGTTTTTGGCCAATAGCATCAGTTT
CACCAGAAGCACGTTCTGCTTCCCAGCTTTCTAGGTCCTCATCTGACCAGAGAGAGCTTGATTTTAAAACCCTTCCCACTTCCAATCGGGAGAA
ACTCCTAGGATAGCAGTGACCTTGAAAGTTTTGGGGTTGTTTTTTGGATGTTGGTGATTTTAAAACAACAACAAAAAAAACACCTCAAGTAGTG
ATATTTCTTTGTAAACAAAATAAAATGTAAAATATTGTTTTGAAACAATTTTTTAACAAAGTTGATCAAATAAGAACTTTCAGGCTGTGATTTT
AAGCATCAGTTCAGACTGAGGGCAGGGTATCCACTCCTGCATGGAGTGTGAGGTTGAAGTTTGTTGGAAGCCCCTGGTAGTTGATAATATTGAG
GTTGTTGAAGGATGAGAGGAAAGGTCGCTTCTTTCTTAGGAGCTGTGATGCTCCACTGTATTGCATAACGAGATAGCACTTGATTCAGACCCCA
GAGCCTGTAGAATAAATGATGTGGAGAGAGTATTGAAAAAGGCTGGATTTATCTGGGGGAATGGCAATGTTATAAAAGGTTCTTCCCTAATTTT
TTAAGAGATAAGCAATTTATAAAGAGATCAAATAAAACTAATCTCTTTCAAAACAGTATTCATTTAGCACTACCCCTGGAAAAATCATCACTAG
ACTGTGACTCCAAATTTAACGAATCGAGGAAAATTTATTAAGCCTCATTCACCTTTCGGACTTTAACAAATCCTTCAAAAGGGAAAATTCACCA
GAGTACCCTCAGATTATTAATGGCAGATGACAGCAGGAAAAAAGAATGTTGAGAATGGCTAAAAATTGATTCCAATTAGTCTTGGCATGGAGAG
GCAACATCTCACCTGGCCAGAGCCCTGCAGCCAGAGTGCTGTCTCCTCTGCCAGTCAGCCAGGGATCTGGGGCTTACAGGCGATTCCTCCAGGC
CTCAATCTTCTCACCTGTGAAATGGGAAGATACCATTAAATTGTCATTTGGCTTCTTTCAAAATCTCAGGTCTAGAATGGAAAGGCATTGAAGG
TGAGTGGAAGAGAAGAAACTGGATGTTAAAATAATAATAATGTAACAGTTATTAATTCACATGGCCAGACCCCAGGGCAGAAGACTAATGGACG
AGAGGATGATTATGTTCCTTTGAAATAAAATGTCAACATTCAAATATTGTTTTTTTCTGTAAAAAATAAGTGAATGGGTCTTGGAAGGAAGTGC
TCCTAATGATGTAGTTTACATATTAATGCTTAATAAACCATTTTATTTACTCAACAAATATTCACTGAGGACTTATAATCAATAATACCAAGCC
CTATTGAATACAGTAATCTAATATACACATAGAATTGAAAAGACAAAATGAACAAAAGAAACAGACAGTATATCATTTCTGTTTCCTCCACTGT
TTTCATTACTAAAGAAACAAGCTGCAGTACACACCCGGCTGAGAGCTGCTTCCCGGGGAGCAGGAGAAGCAGCACTCATTTCACTCAGACCTGG
GTTTGAGTTCTCTGTTTTTGAATGTGACCCAGAGCTCATCGCTTAAATGCGACTGATAATTCGTGCCTGGCCACCCTCATAGCAGCGCTCTGAA
AGAAACATAAAGGAGTGGATACACGTGTGAGCAGGAGCCTAGACATGTGTCAGGTACTTACCTAAACATGGATGTTTAGGTCACTGACATACTG
ACACCATCAAGCTAATTCTTCTCTATTGGTCTCTATGTCAGTAAGGCTACGTGCCAGTAATGTCTATTAAATATTACCCTGTGGTGAGTATCAA
TTTCACCCTAAAATGTTCATTTATTCCCTTTGGTGGTTAAGTTACTTGGACTACCACAGTGATGCCTCTATGTCCCCACACTCAGGAGGTATGA
AAGCGTCTCTACGAAGGAGCAGTGGTCTGGTCTCAGTGTGGCAGCAGACAAAGCCAGGCTCGCACTTAATTACATTGAATTATTTCTGTCTGGT
ATGGCCTCAAAAGGTGCAAAATGATCATAAATATCTTTCGACAGTCACCTTTGGTTTTTGCCCTTGCCAGCACCTGGCCCTTGTAGGTGCTGCT
GGTTTAAGGAACTTGGAGTATTAAGGGGATCCATAAAAGGACTTTGACAGAAGTCTTCCTGAACTGTGCTTAGGGCCAGCAAGGCAGGCAGGGG
CTTGGTCATTTCAGCTGTGCTGTACCCGGATGAGTGGAAACTGTGCCTTCTGATGCATCTCTCCTCCTCTGTTCCCCTCTAAGAACCCCTTGTC
TGTGCTCACATGGATGAGCAACTAGTCTCTTCTCCCACCCCAAAAACCTCCTTAGATTGGGTGGATTCAGTAAAATCAAAGGCTCAGTTAGGCA
ATATAGGAAAAGATGAATTTTTGTGAATATAATTTTCTTTAGTAAAAGGCTTTTCGTCACCTCTTATAATTTCTCAATGTCTTTATGGAAAAAA
TAAGTTTCTCTTGAGCCTTTGGTGCACAAACAGAGTCACCAGCCCTTGGAGGTGCATGCATCTAATGACAGAGCAGCCTGTCACTCGCGAGCAT
CAGGCTCACTTGTTCCTTCCTGTGATTGTCTCTGGGGGGTGGAGGCTTTTGGGAGGCCCTGCGCCATGGTGCAGAGGAAGACCCCTGACCTCTG
GCAGCCCTGGGCAGGCTCTGGGTGTGGCTTGGCAGAGAAGGATGGACATGTCAGGAAGCATATTAGCTTAATAGATGAAGTGTGTGACCAGTGT
AATTCCGTTTTATGGATTTATATTTATCTTGCATATAAATCACGGTGTCGCCTATATATGTTTGCCCATTTAATAAAAAACACATATTCCAAAT
GTTAAAACATAAAATATACCAGCAACAATAGCCAATTTGAAATATATGTTTTCAAAGTAACATTCATAATACCAATGAAGACATAAATACTTAG
AAATGAAGAAAACCAGCAAGCCATAGTGAGGCAAGAAAGAAAACATGGGAGATTGGAGAGGTAGAGAGGTCTGGATGCCGTCCATGGGCCTCTG
CCCAAATTAGTGTATCATTTCAACATAAAACTGGCTTGTAAATTTTACTGGAATCTGGTAACCATGTATCATAAAGTATCTGGATATATACATG
AGATAATTGGCAACTTTTTAAAAGAAGAATAATGTCAAGTGATGTATGAAAATGTAGCTGAAATGGATGGAACTCTTTGAAGAATAGACAAACA
AATCAATAATAGAGATGGATACCCTACTGAAGACCCCATGATAAATCAGCATGAAATATCCGAGGCAGGAGGAAGCATAAGGCAGGAACGGTAT
TGGAAGAATTGATCAGGCATTTAAGATAATCAGTTTACATTATCATCTCCTTATACACTAGGATAAATTGCCGGGAAATGAAAGAGTTATATTT
TTATAACTCCAACCACAATAACTGATGTAAATATAGATCCAGCTATGCAGGATACAAAGTTGTATCTTCAATTATGGAAAGAGATGACATAGAA
CAATTGAGAATGACTACAAATGGCCCACACATCAGCATTTACCTCCATAATTTCTAAATTTTCTACAGCAGTCAGGGGTGCTTTGATTTTCAGG
AGTTTGTTGTATGCTCAGGGATAAGGGGCTCTGGGTTTTTCCTCCACATTGCCCAGATCTGCTGTAGGCTCTTGGGGGCCACATTTTTGTATCT
CCGTTCACTGTGCTGGTCCATGAAGTCAGATGTTAGATCACACAGTTCTTTGCGTCTCAGAGGGCTGCTGTTAGGGACTGACTGAGGTAGCAGA
TTTGGGAGTATTATGAAAAGTTAAAAGGACTTGTCAAAGGGACATCAATACTTAATCCCAGTCCAGCCTCTGGTGATGGTGGTGGTGCCTGTGC
ATTTGGACTTAAAGAGGATGATGTGTGGTGAGCAGCTGTTGGAGGAACAAGCATTTCCAGGTGGCTGGTCATTGGCATTGCTCCGGCTGCCTCT
GCCTCTCTGGAAGTGTTGCTGGGAAGATTAGAATTAATCTCTAGGAAGGGCCTGGCTCTTCTAGGCACTTAACAAATGTCACATTTAACATTGG
ACGCGACTGAACCCTTTAAACATAAGCCTTTCTAAACTGGCCTCTCTGTCTTTGACTTTAGTCATTAAAGAAGAAACTAATCACAGTGAAATGG
CAGAAGACCTGTGCAAGATAGGATCAGAGAGATCTCTCGTGCTGCACAGACTAGCAAGTAACGTCGCCAAACGTAAGAGCTCTATGCCTCAGAA
ATTTCTTGGTAAGAGTTAAATGTTTGCTGTCTCTTAAAAAAAAACTATGTCGGTGTTTTAGATGCAAGTAGAAATGAGTTGAGGGTGGAAGAAA
GGGAAAAAAATCTTATTTTTTCAAAAGGAAAAATTGGTAAGCTTAACATTCCTTAAATATCTTAGAATTTTTTCCAATAAGTATCTTAAAAATA
ACAAACCTCCCATCAGTTTTTCCTAGATTTGATTTTGCAGCATCTGGGGCCTGCCCTGTGATCTGCCTGTGGACATCGCTCTTAGGGGCGGCTG
CACCAGCGTGCACAGGGTGGAGAGTTTGGCCCTGGCTCGTCCGGGGGACACCACACTGCAGGACACTCCAGGCCTGGCCGGCTTCTCAGAGCTT
CAGATCCTCATTTTTCATATGAAGCTCCTAATGCTCCCCTTATGGGGGACTCTGAAGGGTTAATGGGAGGAATCATACAGTGACTGACCCCTGA
GAAGTGTCCAGTGAAGACAGGGCTTAGCTAGGATTGCTGTTTTGCCTAATGCTCTGCGGGATTAAAAAAAAAAAAGAAGAAGAACAAGACCATT
CGTCTCTCTAGGAGCATTGCCCAGAGTAGGTATTAGACACACCAACACCACCATCCAGCCAGACGCTGCAGGGACAGTGAGCCAGGGTCCGAGT
GGAAAGGCGCTAGGCTTGGGAACCAGCTCAGAGTCAATACAGAGCCACCGCCACTCACCAACTCTGTCAGCTTAGTAAAATGGCTCTGCCCCTA
GAGCCCTGGTTCCATCCTTTAGTATCTCACAGGGTGATTGTGAATATCCCATGACTCCAAGATTGAGAAAACGTTTAGAATCCCTCGGTGTGAA
GGTTAACTCTGTCCGGAAAGAGGACCAGTAAAAGCTTCATGAGGCTGAGATGCACTTTGGAAGAGGAATAGAGTTTCAGCACATTCTAGGTGTT
GGAGGAATGGGGGAATCTAGGCACATGTTTAAAATCAATGAGAAACCAGAATGCTGACCATGAGGGTTGGAGTGGGGGCCTAAGGACATGACGG
AGGAGCAGGGTGTGTTCCCAGCTTAACTCAGGTACCCATGGGGAAGCAGGAAAAGTGAAGGTGTCCTAGCCAGCTCTGCCACAGGATGAATGGC
TTCAGATGCCAGGTGAGCGAGGGACCCTTCATTCAGTCAGCAGGAAAGAAGCACTGGCATATTTTTTATGAGAACAAAGGCTAGGATAGTAAAG
ACAGCAAGTACCAAAAAATGACTGGAAAAGGGAGACTGTGGAGGCAGTGGCAGCAGGCATCGAAAGAACCCCTTCTCAACCGGAAGGGGTGGTG
TCAGAGGAACATAGGGCTGGGGGCAGGGATTAGGTGAGGGAAACCATGAGTCACACTGATTCTAGAGTAGTGTGCCCTTGATGAAAAGGATAAC
ACCAGGTTCTAGGAAAAGATGGGGTTCTGTTTTTGACGTGTTGATTTTCAAGGACTTCTGGTGTTTGTGACACATGGGGAAATTGTGGTGGGAG
AGAGGTGGGGCCAGAACAGGGGCTGGTGAGGCCAAGGGTCCCAGAGGGCACCTGTTGACCTGCAGGATGACATGAAGGGGGAAGGACAGAGGCA
AGGCCAAGTCCTGGGCACCAGCCTCCCTCTTGCAGCTTCAAATAGGGCTCCATTTTGACCTTTTGATTAATTAGAGGTTTGTCATACGTTGGGG
GTTGAGAGGAGCAAGGGAGAGAAGGATTCAGTGTACAAAAAGAATGAAAGCCACTGGCTCAGCCAGTGGCGAGTTGTCCACACACACATGAGCC
TTTGGACCATGAGAACGAGGGAGGCCTTGCCTTCCTGAACCGACTAGGAGTGAGGTCCTGTGCTGACCGTAAGCAGTGGGATTCCCACAGCACT
GGGCACAGAGCCCACGGGCTGCCTCCTGAGCAGCCAGCATCTGCCTGGGGTGGACACAGTGACAGAGAGATGGGTGGTGACTCGGGTATGGGCA
GAGATAAGGCAGCAAGTGTGTGCAAGCGCAGTGAAGGGTTACTGACCTTAAGAAGCAGGGATGGCGTCCTCTGTCAGGTGAGGAGCCTGGAGAA
TGCTTTCGTGAATGAACGTTTGCAGCCCCTTTTAGCTTTTCCACACTTGAAACCAAAGCAGAAATTCATCTGTGAAACTCTACTGGAGCCACTC
CCCAACCCCCACCCTTGTGAGACCACAATGTGGGCGTTGGCTTGAGATGCTTCTGTGTTAGTAGAAGAAATAAACAACACAGTGCTCTGATGAG
GCAAAGCGAAGATGAAAAAGGAGTTCCCAGGGGACATAGTAGGAACAGTGGACGAGGGTAGCAGAAGAGGAGTTTCGAGCAAAAGACTCACAAG
CAGCTGCATAATCTGTTGGTGCTTGGCAGTTCATTTGTAAAAATGATGCCTCTTCC
HUMAN SEQUENCE - mRNA
GAATTCCGGCGTCGCGGACGCATCCCAGTCTGGGCGGGACGCTCGGCCGCGGCGAGGCGGGCAAGCCTGGCAGGGCAGAGGGAGCCCCGGCTCC
GAGGTTGCTCTTCGCCCCCGAGGATCAGTCTTGGCCCCAAAGCGCGACGCACAAATCCACATAACCTGAGGACCATGGATGCTGATGAGGGTCA
AGACATGTCCCAAGTTTCAGGGAAGGAAAGCCCCCCTGTAAGCGATACTCCAGATGAGGGCGATGAGCCCATGCCGATCCCCGAGGACCTCTCC
ACCACCTCGGGAGGACAGCAAAGCTCCAAGAGTGACAGAGTCGTGGCCAGTAATGTTAAAGTAGAGACTCAGAGTGATGAAGAGAATGGGCGTG
CCTGTGAAATGAATGCGGAAGAATGTGCCGAGGATTTACGAATGCTTGATGCCTCGGGAGAGAAAATGAATGGCTCCCACAGGGACCAAGGCAG
CTCGGCTTTGTCGGGAGTTGGAGGCATTCGACTTCCTAACGGAAAACTAAAGTGTGATATCTGTGGGATCATTTGCATCGGGCCCAATGTGCTC
ATGGTTCACAAAAGAAGCCACACTGGAGAACGGCCCTTCCAGTGCAATCAGTGCGGGGCCTCATTCACCCAGAAGGGCAACCTGCTCCGGCACA
TCAAGCTGCATTCCGGGGAGAAGCCCTTCAAATGCCACCTCTGCAACTACGCCTGCCGCCGGAGGGACGCCCTCACTGGCCACCTGAGCACGCA
CTCCGTTGGTAAACCTCACAAATGTGGATATTGTGGCCGAAGCTATAAACAGCGAAGCTCTTTAGAGGAACATAAAGAGCSCTCCCACAACTAC
TTGGAAAGCATGGGCCTTCCGGGCACACTGTACCCAGTCATTAAAGAAGAAACTAATCACAGTGAAATGGCAGAAGACCTGTGCAAGATAGGAT
CAGAGAGATCTCTCGTGCTGGACAGACTAGCAAGTAACGTCGCCAAACGTAAGAGCTCTATGCCTCAGAAATTTCTTGGGGACAAGGGCCTGTC
CGACACCCCCTACGACAGCAGCGCCAGCTACGAGAAGGAGAACGAAATGATGAAGTCCCACGTGATGGACCAAGCCATCAACAACGCCATCAAC
TACCTGGGGGCCGAGTCCCTGCGCCCGCTGGTGCAGACGCCCCCGGGCGGTTCCGAGGTGGTCCCGGTCATCAGCCCGATGTACCAGCTGCACA
AGCCGCTCGCGGAGGGCACCCCGCGCTCCAACCACTCGGCCCAGGACAGCGCCGTGGAGAACCTGCTGCTGCTCTCCAAGGCCAAGTTGGTGCC
CTCGGAGCCCGAGGCGTCCCCGAGCAACAGCTGTCAAGACTCCACGGACACCGAGAGCAACAACGAGGAGCAGCGCAGCGGTCTCATCTACCTG
ACCAACCACATCGCCCCGCACGCGCGCAACGGCTTGTCGCTCAAGGAGGAGCACCGCGCCTACGACCTGCTGCGCGCCGCCTCCGAGAACTCGC
AGGACGCGCTCCGCGTGGTCAGCACCAGCGGGGAGCAGATGAAGGTGTACAAGTGCGAACACTGCCGGGT3CTCTTCCTGGATCACGTCATGTA
CACCATCCACATGGGCTGCCACGGCTTCCGTGATCCTTTTGAGTGCAACATGTGCGGCTACCACAGCCAGGACCGGTACGAGTTCTCGTCGCAC
ATAACGCGAGGGGAGCACCGCTTCCACATGAGCTAAAGCCCTCCCGCGCCCCCACCCCAGACCCCGAGCCACCCCAGGAAAAGCACAAGGACTG
CCGCCTTCTCGCTCCCGCCAGCACCATAGACTGGACTGGACCAGACAATGTTGTGTTTGGATTTGTAACTGTTTTTTGTTTTTTGTTTGAGTTG
GTTGATTGGGGTTTGATTTGCTTTTGAAAAGATTTTTATTTTTAGAGGCAGGGCTGCATTGGGACCATCCAGAACTGCTACCTTCCTAGATGTT
TCCCCAGACGCTGGCTGAGATTCCCTCACCTGTCGCTTCCTAGAATCCCCTTCTCCAAACGATTAGTCTAAATTTTCAGAGAGAAATAGATAAA
ACACGCCACAGCCTGGGAAGGAGCGTGCTCTACCCTGTGCTAAGCACGGGGTTCGCGCACCAGGTGTCTTTTTCCAGTCCCCAGAAGCAGAGAG
CACAGCCCCTGCTGTGTGGGTCTGCAGGTGAGCAGACAGGACAGGTGTGCCGCCACCCAAGTGCCAAGACACAGCAGGGCCAACAACCTGTGCC
CAGGCCAGCTTCGAGCTACATGCATCTAGGGCGGAGAGGCTGCACTTGTGAGAGAAAATACTTATTTCAAGTCATATTCTGCGGTAGGAAAATG
ATTGGGTTGGGGAAAGTCGGTGTCTGTCAGACTGCCCTGGGTGGAGGGAGACGCCGGGTTAGAGCCTTTGGGATCGTCCTGGATTCACTGGCTT
GGGGGAGGCTGTTCAGATGGCCTGAGCCTCCCGAGGCTTGCTGCCCCGTAGGAGGAGACTGTCTTCCCGTGGGCATATCTGGGGAGCCCTGTTC
CCCGCTTTTTCACTCCCATACCTTTAATGGCCCCCAAAATCTGTCACTACAATTTAAACACCAGTCCCGAAATTTGGATCTTCTTTCTTTTTGA
ATCTCTCAAACGGCAACATTCCTCAGAAACCAAAGCTTTATTTCAAATCTCTTCCTTCCCTGGCTGGTTCCATCTAGTACCAGAGGCCTCTTTT
CCTGAAGAAATCCAATCCTAGCCCTCATTTTAATTATGTACATCTGTTTGTAGCCACAAGCCTGAATTTCTCAGTGTTGGTAAGTTTCTTTACC
TACCCTCACTATATATTATTCTCGTTTTAAAACCCATAAAGGAGTGATTTAGAACATCATTAATTTTCCAACTCAATGAAAATATGTGAAGCCC
AGCATCTCTGTTGCTAACACACAGAGCTCACCTGTTGAAACCCAAGCTTTCAAACATGTTGAAGCTCTTTACTGTAAAGGCAAGCCAGCATGTG
TGTCCACACATACATAGGATGGCTGGCTCTGCACCTGTAGGATATTGGAATGCACAGGGCAATTGAGGGNCTGAGCCAGACCTTCGGAGAGTAA
TGCCACCACATCCCCTAGGAAAGACCAGGCAAATGGCACTGCACGTGAGAACCCCGCCCATCCGTGCTATGACATGGAGGCACTGAAGCCCGAG
GAAGGTGTCTGGAGATTCTAATCCCAACAAGCAAGGGTCTCCTTCAAGATTAATGCTATCAATCATTAAGGTCATTACTCTCAACCACCTAGGC
AATGAAGAATATACCATTTCAAATATTTACAGTACTTGTCTTCACCAACACTGTCCCAAGGTGAAATGAAGCAACAGAGAGGAAATTGTACATA
AGTACCTCAGCATTTAATCCAAACAGGGGTTCTTAGTCTCAGCACTATGACATTTTGGGCTGACTACTTATTTGTTAGGCGGGAGCTCTCCTGT
GCATTGTAGGATAATTAGCAGTATCCCTGGTGGCTACCCAATAGACGCCAGTAGCACCCCGAATTGACAACCCAAACTCTCCAGACATCACCAA
CTGTCCCCTGCGAGGAGAAATCACTCCTGGGGGAGAACCACTGACCCAAATGAATTC
HUMAN SEQUENCE - CODING
ATGGATGCTGATGAGGGTCAAGACATGTCCCAAGTTTCAGGGAAGGAAAGCCCCCCTGTAAGCGATACTCCAGATGAGGGCGATGAGCCCATGC
CGATCCCCGAGGACCTCTCCACCACCTCGGGAGGACAGCAAAGCTCCAAGAGTGACAGAGTCGTGGCCAGTAATGTTAAAGTAGAGACTCAGAG
TGATGAAGAGAATGGGCGTGCCTGTGAAATGAATGGGGAAGAATGTGCGGAGGATTTACGAATGCTTGATGCCTCGGCAGACAAAATGAATGGC
TCCCACAGGGACCAAGGCAGCTCGGCTTTGTCGGGAGTTGGAGGCATTCGACTTCCTAACGGAAAACTAAAGTGTGATATCTGTGGGATCATTT
GCATCGGGCCCAATGTGCTCATGGTTCACAAAAGAAGCCACACTGGAGAACGGCCCTTCCAGTGCAATCAGTGCGGGGCCTCATTCACCCAGAA
GGGCAACCTGCTCCGGCACATCAAGCTGCATTCCGGGGAGAAGCCCTTCAAATGCCACCTCTGCAACTACGCCTGCCGCCGGAGGGACGCCCTC
ACTGGCCACCTGAGGACGCACTCCGTTGGTAAACCTCACAAATGTGGATATTGTGGCCGAAGCTATAAACAGCGAAGCTCTTTAGAGGAACATA
AAGAGCGCTGCCACAACTACTTGGAAAGCATGGGCCTTCCGGGCACACTGTACCCAGTCATTAAAGAAGAAACTAATCACAGTGAAATGGCAGA
AGACCTGTGCAAGATAGGATCAGAGAGATCTCTCGTGCTGGACAGACTAGCAAGTAACGTCGCCAAACGTAAGAGCTCTATGCCTCAGAAATTT
CTTGGGGACAAGGGCCTGTCCGACACCCCCTACGACAGCAGCGCCAGCTACGAGAAGGAGAACGAAATGATGAAGTCCCACGTGATGGACCAAG
CCATCAACAACGCCATCAACTACCTCGGGGCCGAGTCCCTGCGCCCGCTGGTGCAGACGCCCCCGGGCGGTTCCGAGGTGGTCCCGGTCATCAG
CCCGATGTACCAGCTGCACAAGCCGCTCGCGGAGGGCACCCCGCGCTCCAACCACTCGGCCCAGGACAGCGCCGTGGAGAACCTGCTGCTGCTC
TCCAAGGCCAAGTTGGTGCCCTCGGAGCGCGAGGCGTCCCCGAGCAACAGCTGTCAAGACTCCACGGACACCGAGAGCAACAACGAGGAGCAGC
GCAGCGGTCTCATCTACCTGACCAACCACATCGCCCCGCACGCGCGCAACGGCTTGTCGCTCAAGGAGGAGCACCGCGCCTACGACCTGCTGCG
CGCCGCCTCCGAGAACTCGCAGGACGCGCTCCGCGTGGTCAGCACCAGCGGGGAGCAGATGAAGGTGTACAAGTGCGAACACTGCCGGGTGCTC
TTCCTGGATCACGTCATGTACACCATCCACATGGGCTGCCACGGCTTCCGTGATCCTTTTGAGTGCAACATGTGCGGCTACCACAGCCAGGACC
GGTACGAGTTCTCGTCGCACATAACGCGAGGGGAGCACCGCTTCCACATGAGCTAA

TABLE 26

MOUSE NOMENCLATURE
ICSGNM Tbx21
Celera mCG13239
HUMAN NOMENCLATURE
HGNC TBX21
Celera hCG27200
MOUSE SEQUENCE - GENOMIC
TTGGCAGCAGGGACAAGCAGAAAAACTGGGGTTTTGTCTCCAAAATCACTGAGAAAGGGATTAGAGAAAGCAGAAGCCAAATGGGGTGATATGT
CTAGGGTAGCAGGTGTTCCGAGAGGCCCCAGAGCCCAGGCCAGTGCACACAGGCACAGATATTTCCCCCTGCAGGGCAAGGAAATGGACTGGTA
TTTCAACTTCCAGCTATTGCTTCAGATGCTAAGAACTTGCCTGACCCTGATGCTAACCTCCCCTCCCTGACCCACAGTGTCAGAATCTGGTGCT
GGTGACAGGAAAAAAACAGGCGGGTATTAGGGGAGGGAGGGAGGGAGGTGTGTGTGTGGGAACTAATAGGCTACCTCACCAGCCACCCCACCCC
CAGCCATTTCTAGGTCCTGCACAGAATGCCAGCTGCCCATGGCCCTGGTGGGAGGAGGTTTCCACGGTGCCTTGTGATACACACTGGGTCGCTG
AGAGGGGTGAACTATAGACATCAGGCCTCAGGGAGATCACAGACTTGTGTGTCAGAGTGAATCTGCATCTGGGGTGATAAGTGGGGTTAATCTC
AAGGTCAGATCTAGCCAATCCATTTTCATGCCAACTTGGCCCTGGGTGCCCTGCATGGTGGCAGCTGAGACTCGGATGTCCCCTTTACTAGAGA
TGGTGCCTTGCCTGGCTTGTCTCCCCACTATGGTTATCGGTTTCCAATCATTACCTTTATTTCTATTCCATTACCTAGAGATCTAAGGACCCTG
AACGGTCCTTACTGTTCGCTGTTTTAGGAGACTCAGAAAAGAAATGGGATTGAGAGTGAAGGCTCATGGGAAAGGATGTCCAGCCTGAGGGAGG
GGCGGAGCCCTAACCCATCAGGCATCTCAGATAGATACACAAACCAGACTCTCAGCCTCTTTGCTCTGCTCCCTGGGGTTCATCGCACTGCCCT
ATGCTCTCGGGACTCACCTCAGGCTCTGCCTTTTACAGGCTGTGACTGTCACCATGTTCATAGGAAGAGGGGGGACTAGGAAAGCTCTAAGAAT
AAGCTGGAGGGCCAAAGTAGACTCCAGCAGGACTCAGAGTTGCTAAGGTGATAGAGAGGAGGCAGTTTGGAGAGGCAAGAAACTGGGTGAGTTG
GCTAGCTGTCTTTGCAGCAGTTAACCAGAGACTAGAGAGTGAGAAGGACTTCCATACAGGGTCTCTGGTGTTGGAGTGCAGGGAGTGCAATGTG
GCTAGGGTGGGCTGGGTGAAGGAAGATCTATGCTGTCCTTCCTTTAAGGAGGAATCTGTCCAGAGGGGACTAAAAAACAGAATTCCAAACAACT
CATGAACAGGGCCAGGCCAGCATCTTCTTGTCCCCAGTCCAGCCCCCGCCTCTGAAGCCCCCCCCCCCCCGCACACCTCTGGAAGCCCTCCTCA
GCACCCCCACCCCACCAAAGCCCACAGCCCTGGGGAAGCTGTGTGGCCTTGTCACCACGGACCCAGTGTCGTGCCGGCTGTGGCTTCTTCCTTC
TGACCTATTTTTTAATCATTTGATAAGATCCCCCCTCCCAAACCAAACCCCGGCCCCCGGGCCCGTCAGCTCCACCGCGTGTCAAAACCACCCT
TGTCAGTTTGCGTTGGTTTCTTAGTCCAGTAGCCCCTCCCCTCAGGGCCTGAGAGCGAAGGCTAATTACCCAGCTGCTGAGTGCATTCCCCCCA
CCCCACCCCCTGCTACTCCACCCCCAGCCCCAACAGAGGGGGCCCTGGAGGCCTGCTGCTAGAAGAAGCTGGGGCCTCCTTTCCCTCCTATTGC
AGGGCAGGAAGTCCTTCCTGCTGTCTAACCTCCATTCTTTTCTCTGTCCTGCCCCTAGTGGTGCTGAATGGCAGATGCAAGGACTGATATTCTA
GACTGGAAGGGACTTTGAGGCATCATCTCCTTTAAGGAAAGTAAAAGTCGCAGCCTCCTGACCTGGATTTAGAGATGTCCTCCTGGGTCTCACA
GAATTGTAGGGGATGGTCTCTGCCAAGGGTCTCAATTACAGGACAGAAAACTGAGATGCAGCGAGAGAAAAGGACAGGCCCAAGGTCATGTTCG
GCTGGTGGGGTAGCTAGGATCAGAACCCAGACCTTTCTGTCTTTCTGTCTGTTGCTGGGTCTCCCTCACTCCATTCTGTTTCAGTTTCTCCCTA
CCCGTGTGTGTGTGTGTGTGTGTGTGTGTGTGTGTGTGTGTGTGTGTGTGTGTGTGTTGATACACACTCTATGGGAGCTTGTGGAGGCCACAGG
TCAGTGTTAGGCATCTTCTTCAACTTGGTCTTCAAATACATTTATTTAAACATTTTTTTTCTAATGCGTGCAAGTGTTAGCATACATGTATATA
TGTCATCATATGGATGTGTGGTGCCCTTGGCCGCCAAAAGGGGAGTTACTGATGTTTGTGAACCACCATGTGGATGCTGGGACTGGACCTGGGT
CCTTTGCAAGAGTAGTCAGTGCTCTTAACCATTCAGCCATGTCTCCAACCCTCCCCGCCCACACACACTTTTATTTTTGGAGATGGGGTCTCTC
ACTGAATCTGGAGCTTGCCAGTGTGACTGGTCTGGCTGGCTGGTCACTGGGTCCCAGGAATCCTGCAGCCTCTTTCTTCCCAGAGCTGGGATAA
TAAGTGGATATGTTGTGCTTAACACAGAGGTGTGGGAGAGTTAACTCTGGTCCTCATTTCTTGAAACGAGCCCTTTATTGACTGAGCCCATTTT
TCATTCTGTCTTTGTTTTTGAGACAGAGTCTTACCATGTAGCCCAGGCTGGCCTGGAACTCAGCATCTTCCTACCTTAAACCCCCCCCCCCCAG
AGTGCTGACATTCTAGGCATGTGACACTACACCCACCTTATTTTCTCAGTGACTCAGTCCTTATTCCCTGAGCAAGGACATCACTTCGTGCCCC
TTGCGAGCCTCATCCTCAGGTCTGGGGTTGTCCAGCTTGCTCCATACACCATTAGAGATTGATCACTCCTCCAGCGTGTAGCTGCTGTCCCCTC
TTCCCTCAGACTATCAGCTGCTCCATATCAACTTACCCTGTCACATTATCGAGAAGGGCAAAGCCAAGCGTCTGTGTGTGAGAGAGGCCTCAAA
AAGGAGGCCCTGCACCAGGTATACAGGCATACAATAGAGTGCAGTTGGACTGGCCTTGAGGCTCTGACAGACTTGGAGATGCAGAGACAGACAG
ACAGACAGACAGACAGACAGACAGACAGATACAAATCCATGATAGAGATCACAGTAAGTTCTTGGAGCCAGGGATAACAGGAATACTCGAAAAG
GCAGGGACAGAGAGAGACCCTATTATTCGACATCAGCAGGCATATTTGTTATGAAATATAATGCGGCAAGGGGGTACCTGGCACAACCATGCCA
AGGTGTGTCCATGAGTAACTACACCACACAACAGAGTTGGTATAAAGAAAGTTTACTGGGAGAAGGGAGAAGAGCAAGGACTTGGAGCAGGGGT
AGAGGCAGGAGAGTGGACATGTAGACAGACGGACAGATGGGAGCAGACTCATGGGGGCAGAGAAGTGGCCAGGAGAGGGAGCAGAGAAGGACAG
GAAGAGGGAAGAAAGAATGCTGGGAACACGTGGGAACACAGAGAGAACTGGGNNNNNNNNNNNNNNNNNNNNATGTAAGCCACTGATAGGTTCC
TGGGAGGAGCCTAGTGGAACTGCTGTAAGCTAACAGTATTTATTGTTGTTTTAGTTTTAATTAATTTTCAGATAGAGTATACTGTAGTCCAGGC
TGGTCTTCAACTTGATAGGTAGGCCAGGCTAGCCTTTAACTTGATCAGGAGTTTGAGATGACCTTGACTTCCTGGTCATCCTGTCTCTACCTCT
CAAGCGCTGGCATTATAGACAAGTCCCACCAAGGCTGACAAGACAGGAGCGGTTCCACAGATCCAGACAGAGGCAGATGCGGCCAAGACCCACG
AAGCTGCAGACAGGACACAAGGCTCTACAGACATGTGCAGGCCACCACCACCAAGGCCAAGAAACACCAACCGAAAAAGCTGTAGTCCTGGTGC
TTAATCCCAGCACTCGATAAGCAGAAGCCGGCAGATCTCCGTGAGTTCGAAGCCAGTCTGGCCTACATAGTTGTAAGCCAGCCAGTGCTACAGT
CTCAGAAAATAAAGGAACGAAGAAGACAGAGAGAGCTACAGACAGACAGACAGGCTCAAGAGCCTGGGAGAAAGCAGGGCTCATGCAGATGGTG
AGGTTAGCTTGCCCGGCCTGGCACTGCTGGACTGTTCAGGAAGCTAGATAGCTTCCCGAGGAGACAGGGACAGAGGAGTCCAGCTACACAGGAG
TTAAGGTTGGCTCTCCAGCCTTGCCCTGGCTTCTCTCTCCCTTCCCTCCTCCCAGGCTCCCTTTACCCAAGGCCCTGGCATTCCCCCACCCCCA
CCCTTGGTAACCAGACCCTCCTTCCTGCTGAGATTAAGGCCTGCCTGCCAGATTCTGGGGTGGGGGGGGCAACACCTCCCCTAGGGCCTTCCTA
AAGCTTGTGCTCACCCTCAGTACCCTGGCAGCCTCTGTGATTACAGGGTTGCTCCAGTGATAGCATTAAGGGAACCCTCATCCCTAGACATGGG
GTGCCTGTGGAAAAGTTAGGGGGGAAAGGACCGTAGTTTGGGACTGAGTTGTTGTTCAGGACCAGAGGGTAAACGGTACCCATCTTAAGAGGTA
TTCAGAGTGCCAAAGGGTGACCCTCAACCCGGACATGAGATCCTGAAGCCCTTGACCTTGGAGTAAACCCCGGGTGCTGGCTACTGGGGATTCC
GACCTCTTGCAGCTGTCGCGGTTGGGGGTGGGGTGTGCTGTCCAGGTGCTGGCCACTGCCCTGCCCTCTCCCTGCTAAGCCGTCGGTTCCGGCC
ATGGGCATAGCCATGCTCTCTGAGGACTGGCTGGTCTGGGCCCTGAGCTGGGAGGGCTCCAGGGTGGGTGGATAACAGTGGGATGTGAGCAGAC
CTGAAGGCAGGATTGAGGGGTTATCTTGAGGAGGACTCTGTGTTTGGCATGTTTATGTACATGGGAGGAACCGGTGTGTGCAGGAACCTATCTG
GGTTGCCAGTCTGCCTCTGGGCATAAGTGGCCTGACGTACAGGCGAGATCTGAAGTGCATGGTCACTCACCTAGCTGGGAGCTCTTGGGGGAGA
AGGCTAATTCTAAGTTTTCAGAGTCCCCAGGCCTAGTTGATTACAACCGGCAGGAAATGGTGTACGGGGTCAGGAGGGAAGCAGGCTTGCCCCT
ACTCACTGAGAGGAGTCTCCTGAACAGGTTCGCTTCTGGCCCCAGGTCCTAGTGGAGGAGGGGGTGAGCATGAGGGGTCTTTTGTGGAGGACCC
ACAAAGCTAGATGGAAGACAGGGAAGATGTGGGTGGCTGGAAGCAAGAGACAGGCAGACAAGGCAGCACAGCCTGGGCCTCCATTCCCTTCCTT
CCTCAGGGCAAGGCTCTCGGCCTTTAGAGTCCAAATGAATCTTTTTCACTGACAAGGAAAGCAGAGCTGTCAGGATGCAGGGCAAAGGTCACCC
AGAGAGTGAGGCTTGGACTTGGGGGGTTCCCACCAAGCCACCTGTGGAAGATATTGTTTCCCTTTGTCAGGCTTTTGATACCCCAAAGCCTTTT
AGCTTCTGAGGTCTTTGCTTTCAAGGGCATCCCCCTTCCTTGTTCTCTCCCCCCCCCCAGGGGCCTGAGACTTTCTCACTGAGTCACAGCTAAG
GCTCTGACTTCATGGTTTCTTGACTCTGAATCTCTCCTGAGCATATCCTGAGCCAGGGACTCCTTCCTCTGAGCGCCTCGTTCAATCACTGTTC
TGGCCTCTGTGGTTCCAAGGCAGAGACTTGGCTCCTGGTGCCCTGGGCTCTGGGCCACCAGCCCTACCCACCCCCCACTCCGTTTCTATCCTTC
ACTGCAACCCTGGGATGGAGGACTTGGTCCTGCACACATGTTCAGGAGAGGATCAGGCCCACTGATGCACCTGAGAAGGGAGAATCTGCCCGTA
GAGTGTCCACCAGGGCACTGGAACGCCTGCGATGTGGCTTCCCTTGCCTTTCATCCACCTATTCCCTGTCCCTGGAAGTTTCTGACTTGGAGAT
AGCAGAGAGGAAGAGGGAGGATCCTGGACGTGGGATGTGAGGCATCCTCAAAGGCTATTGTCACCTTAAGTTCTTAACTCAAGACACTGTCGGG
CCCCAGCATCAACATTGTCTAAGGAGCAAGTCCCAGAAACCCACATGACAGCTTGCTGGGACAGGTGGTGGCTTAGAGGAATGACCAGGGCTGT
GTGGCCTTGGGAGAGAATCCCTGGTACTCTGTGTCTTAGACTTGAAAGAAGTCTTGCGCACTTGTAGAACAGCACATGGTGAGGAGAACCAGTG
CCAAGTTGGAGGGCTTAGGCTCAGCCCTAAGCCTGGGGTGGGAGGTGGCAGTCTCTCCCCGCTGCTCCCTGTTTCCCTAATCTTTTCTAAGAGA
CTATCAGACCTTTTCTTTTGTGTGGTGCTGGGGATGGAGCTACAGTCTCCTACATGCCAAGTGTGTCACATATGTGCGTGCATATATACCTGCT
ATACCACCATGCCCTCATCCCTTGGCTAGTTTTCATTTACTTGACTTCTGGAAAGAACGCTGGGCTGGTCATCTACCTCAGACCCTCCGCCCTA
GCTCTGTGGCTAACAGGGTGTGTGACTTCTGAAGTGTTCTGTCTCCCACGTTCCCCAGGGCCAGCCTATGGGTAGCACGTACTGGCAGGATCAG
AGAGGGGCTGGGAGCAGCAGCCACAGGGCAGAGAGGGGATCCCAAGGGGGCCTCCTTTCCTCAATCCTGGCTTCCCACCTCTCTAGCCCTGGAG
ACTGGGAAGGGGGGTTGAGGTGGCAGCAGGAGGTGTCACTGTCGGGGTTCTTCTTCTCACCCAAGCTGGCAGAGGGGCGGGACTGGCAGTGGTG
ACACCAGGGCCACTCAGCCCCCGCTTTTCACAAGCACTTTCTTTTTTGGGTGAAAGAAATTAGAGGGAGTGGGGGGGTGAGAGTGGTGAGAATC
TGAGGTTAATAAGGCAGGCTCTATCGACCCATCAGGTTATGGGGAAACCATTCTCGGGCCCCCCTCACTCCAAAGATGCACTTCCAGGACCACC
CAGCTGTGATAGTCATTCCCTTCTGCTCTGAAGAGCTTGTTGGGAGCATAGAACATTTTGTTCCGAAGCAGGGGGATGGCTCACTTGACCTGAG
GATCAGCGTGGAACATCAGGGATGGAAAGGCTGAGGAACAAGACTCCATTTGGCCTTTCACAATGGGACACTCAGCAAAAATGCTCCCCCAAAG
AGTTGTCGGTGTTCAAAAATTTTACCTCCTGGGGAAATCAGTCATTTGAATGAATCCCAGAAATCTATTTTCCACCATAGGGAGCCAAGGGTGC
AAAGGTCACCTTCTTTCAAGTCCTGGTGTCTCATTTTGACCAACAACACACAGAAGCCTTTATGGGCGAGTAGGGGGTGAGGGAGTGAGGAGGA
GGCCAGACCTCGATATCTGCTGCTGTCATGGATCCAGGCCTCATTCTCAAGAGCTGTGCTGATTGTCTGCCCCCACCTTTCCCTGGGTCATGTC
TGCCTCCTGGGGTCCCTAACTTCACCTTCCCCTGGCAGCCTGTCTCTTGGTTCAGCTCTAGTTGGAAGGAGAGTGTCCCCCTGGGTCCCTACGG
GTGAAGTCCTATTGGGCTGGGACAGAGATGTAGAGATGAAGTCACACCCCAGGGGAGAAGTGGGGTGGGGCAGAGATGAGGGAAAGAAATCTCT
TTTTAACGAAATAAAACTGCATGGAAGTATTTTTTGAAGCATTCTTTAAGATGTAAATCTTTATATATTTGACATGCAAAAAAAAAATCCATTA
CACCGTGCTGAGTGAAGAAAAAAACCAAGAACCCACGTGGTAAAGCGGACTGCGAAGTTGATGTAAAATTAGACATCTGTGTGTGTGTACAAAA
GACAGAGACACAGGAGGGTCTATACAAGGGAGGTATCCAGAAGGGTTTTCTTAAAATGTTAATACGGGAGCTAGTATGGGAGGGTGGTGGTTTG
TCTGACTTCTGTTTTTCTTCACTGCCTAGATTTTCTGTACAGAGGATGATTTTAGTCCTGAGGCCAGCAAGATGGCTCAGCAGTTAAAGGCACT
TGTTGTCCAAGCCTGATGACCTGAGTTCCAGACTCAGAACCCACATAAGGATGGAAAGAAAGGACTGACCCCACACAGCTGGACTCTGAACTCT
AAACATGCACCATGGCACAGATGTCCACACACAATAACAAGTAACTTGCTTCCTGGCGGTGACGCCCTCCCTCTGAGATTATGCCTCACTCGCA
AGGGTCTCCTTCATCACTCTCCAGAAGAGGAGAAGAGGAAACACAAGAAAAAGCGCCTGGTGCAGAGCCCCAATTCCTACTTTATGGATGTGAT
ATGCCCAGGATGATATAAAATCACCAGTCTTTTTTTAAAATTAATTTATTTATTATATGTAAGTACACTGTAGCTGTCTTCAGACACTCCAGAA
GAGGGTGCCAGATCTCATTACCGATGGTTGTGAGCTACCATGTGGTTGCTGGGATTTGAACTCAGGACCTTCGGAAGAGCAGTCAATGCTCTTA
ACCGCTGAGCCATCTCTCCAGCCCAAGTAACTCTTAAACGATACTTTACTTCTTATTTACTTACTTTTGAGATAAGATCCCCTGTAACCCAGGC
TGGCCCAAATTGTAGCTGAAGATGACCTTAAACTTCTGATCCTCTACCAACCCTCTCAAGTGCTGGGATGACAGGCATGTGCCACCATGCCCCA
TCTATACAAAGCTGGGGGTGGAACCAGAAGCGTGGGTGTATGCTAAGCAGGCACTCCATCAGTTGAACTATATCCCAGACCCCGGGGATGCTTT
TTATTTCAAAAGAAAACTCCCAATGGATTCATTTTCAGTTAGGAGGTGAGATTCTTCTCAAAATGCAGGACTTACCGTAGTTATTGGAATAGAA
CAGCGGAGGAAGGACTGTCTGTCTCCATCAGAGCAGGTGCTGGGCTCCCTGTCAGCCAGCTTGGACTACATACCTTTGTTTTTTTCTATAATAG
AATGGAAACAGAGGCACACAGACACCGGTTGGTATAAGGTACACAGCCCCTACCCCCAATACGAATATTTATTTCCCCAGCCTACCCCATCAGG
CCTGAGGGACTTTCAGGCAAAGGAAGTGACTTACCCAGGTCCTACACACCCGAGTCACAGTCAGAGTCAGCCCTGGGACTCTGAACACTGACAG
CCAAGCGCTACTTCCCGTGTCTTTCGCTAGCTGTTACGCTGCCACGTGAACGAGCTTTTGTGGGAGCATTTCAGCCAGCCCAGCCCACCGTGGG
CTGCTTTGGGTGCAATCTTCTTGGGAGACAGCTTCCAAGTTTCCTCCTGGGCTAGAGGGACAAGCATCATCCTCGGGCCTGGGGGGTGGGGTGG
GAGGCTAGCGATGGGGGGTGGGGGCGGCGGACAAGAGACAGCGAGCAAACACTTTGTGTAAAGTCACAAAGCCTGGGCATACAGGAGGCAGCAA
CAAATATCTGTTGACTATGGATAGTCTAAGTAGCTCTGAGCCTGCCAGAGGCGTGAGAATGCTCAGATGGTCCCATCTGGGGAAAGGGCCTTTG
CTGTGCCTTCCAGGACTGGCGGAACTTCCTGGGGGAGAGAAACCGTTTCTCTCCTCGAAGTTTCAGAGTTTCAGGTGGCAGGTTGACTCTTTCC
CAGATTGGGGCGACAAGAGACTTACACTTAGGAGTGGGGGTCGGGGCTTCAGTCCTCACCAAAAGCGAATTCGCGCTGTATTAGCCACGAGGGG
GCGGGGCGGGGCGGGGCCAAGGTGGCGGGGTGAGGGGACCCAAGGAGCTTCATAAAGCCACAGCAAAGGCCGCCGCGCCGCTAGTGACAGCGCG
GGCTGCAGAGAAAGCCCAGGAGCAGCTGCGCGCCTATCCAACGCGCGCTCAGGAGCCAAGCGTCCCAGCTCCCGCTCCAGTGAAGTTTCATTGG
TCTTCGGACGCCGCCCCCGTCGCCCCAGCCCTAAGGACCCTCGGGTCTCTTCGACGGCTGCTGGAAGGCGCCCAGCCCGCCTCGGATGGGCATC
GTGGAGCCGGGCTGCGGAGACATGCTGACCGGCACCGAGCCGATGCCGAGTGACGAGGGCCGGGGGCCCGGAGCGGACCAACAGCATCGTTTCT
TCTATCCCGAGCCGGGCGCACAGGACCCGACCGATCGCCGCGCAGGTAGCAGCCTGGGGACGCCCTACTCTGGGGGCGCCCTGGTGCCTGCCGC
GCCGGGTCGCTTCCTTGGATCCTTCGCCTACCCGCCCCGGGCTCAGGTGGCTGGCTTTCCCGGGCCTGGCGAGTTCTTCCCGCCGCCCGCGGGT
GCGGAGGGCTACCCGCCCGTGGATGGCTACCCTGCCCCTGACCCGCGCGCGGGGCTCTACCCAGGGCCGCGCGAGGACTACGCATTGCCCGCGG
GGTTGGAGGTGTCTGGGAAGCTGAGAGTCGCGCTCAGCAACCACCTGTTGTGGTCCAAGTTCAACCAGCACCAGACAGAGATGATCATCACTAA
GCAAGGACGGTGAGTGAGGTGCGCGCCGAGCGCAGCGAGCTCGCAGGGCGGAGCCGGCGGGAGTCTGGCCTCGGTGGCCCGCCGGAACAACGAC
TTTCCTCTTTTTTTTTTTTTTTTTTTTTGAAATCAAAATTTGAAGCTGCAGGTTGCTGAGTCTTTGTGTTCGTAGACTGAGCTTCAGTTTTAGG
AGACGGGGGAGCAGGGTTGCGGGGAGGGCACCGCAAGCTCCGGAGAGCCGCGGGTGCTGAGGGTATCAGAGGGCTCAAAAAGGATGATGCTGTC
GGCGCAATTAGAAGTTACTTTGGTGCAGAGGAAAGGATGACTCTCGCTTCACCGAGAGTTGGCCGGTGGGGGGTGGGGGGTGGGGGACTGGAGG
GGGTTAATTTGCTCCTTGCCTCAGTCTGGGATCTCAGTGGGCAGCGAGGAGTGTGTGTTATAGACCTGACTGACCGCCTTTCGGTCCTTAGGTA
TTCGGGAAATTGAAGTGTTGGGGGCAGAATATTTTTTGAGATCTCAGTGTGCCTGTTCCTTTGCTACTGCTGTGCTTAGGGTAGCTTTGGTGTA
TGGGGGAGGGGGGGAGCAGACTGGGATTTGGTGACATGGAGAAGCGGTCGCCAAGTTTCTTTTCCGGTCTTACTTTGAGATCATATGTCTAGAG
AGGGAGAGAGAGAGAGAGAGAGAGAGAGGAGTGTGTGTGTGTGTGTGTGTGTGTGTGTGTGTGTGTGTGTTGTGTAGAGAGGTGGGAAGCAGCA
AAACTTATAAAGGCACAAATAAATGCCGAGGGCCTGAACGTGCCAAGTTCAAGACCAGTTGCAGCTGGTGTCCAGTCTTGCACTGTGCTCTTTG
AGGCTAGTCCCTAAAAATATGTCCACTTTCTCACCCAGCTATTCATCAGTCCAGCCTGGGGGAAGATGGAGGGGGTCCCCTAAGCCTGTTGCAT
TAGTGGTATGGTTTGTGGCTCAGCCTGCTGCTTCCGGATCGAGTCTCTTTGCTGAAGAAAGAGAAACTTGCAGTTTCTGAGGGGTCATTTATAG
TATGCAACACCTCGAGTGGACCCTCTTGTGTCTTATGGGAAGAGGCAGCTACCTTTCCCAGTGAGGACCAGCCAGAACTTTGCCATTAGTGAGT
TAGGATGTGCGGTATTAGGATGGAGACTCAAAATGTAATTGAGAGATTGCAAGGGTGGGGTTTCTGGGAGGGGGACAGAAACCCTTGCCGGATC
CCAGGAGGCACTGCTAGAGTATATTCCCTTCGGATGATGTGGGGAGAGGGCAGATAGCTGCTAACTACAGCTGGTTTCAGGCTGCTGAGTTCTT
CCTCGCGGGGATGAACTGAGAATAAAACATTTGATTGGTTTTTGGCTCATCCTTCATCCGCAAAGACTTCTGAGGAGTTTCCTCTGCTCCCCCA
ATCCTTCTCTGAGTGTCTCTGGCACTGGCCAGGTTGCTGGTCCTAGAGAATGTCTGAACTGGAAGGATCCAGAAGTCCCTGTCTCTCACTGCAT
AGAGAAACAGACCTAGAGACAGCAGGGGACTCTTCGGAGGTTCCCCAGCTGGGACCAGGCCTCAGATGGCACCTGTCTGCTCCTTTTTTCTCCA
TTCTCCACAGCCACCTACACTGTCTGAAAGCCCTGCGTGGTACACCCAAGTGCTTTCGCTTCCATTATTTCTAGGCGAGCCAGGCAGGGCTGGC
CCGTTTCATACCCACGAATTCTGAGTTGCTGGGATAACCAGTGGCTTACGCTAGTCCCGCAGTAGACTAGGTGGGGCCTCTAACCATGGGACTT
TCTAGTATGTCAGCAACAGGAGGCCCTCTCATCCCGGGGCCTGGCCAGTCCTCTCAATGCCTTGTCAGGCCTCAGCTCCTCCAGTCAGAGAGAG
CATCAGCCACAACCTCAGTGATGGCTGAGCTGGGCTGGGGCCTGTCTGTCTGTGGGCTGGGTTGCTAGGGGAGGCATTTGGAGGGACACACTCT
ATATCACTGGGGCCTGTCTGTCTGTGGGCTGGGTTGCTAGGGGAGGCATTTGGAGGGACACACTCTATATCACTGGGGCCTGTCTGTCTGTGGG
CTGGGTTGCTAGGGGAGGTATTTGGAGGGACACACTCTTTATCATTGGCCTGGCTCTATGGACTCCTCTCCTGTGTGCGCCTATAGGATTTTTT
TCCCATGACCACTTTTGCTCTCTCCCGTTTGGATCGGTGCTCTTATTATTTTAAGTGCTGTAATTGGAACCTGGGGCCTAGTGTATACTAGTCA
TGTTTTTTTGTTTGTTTGTTTGTTTTAAAGCCAGCTGTGGTAGTGCACCCCTTAAGTAATGCACACGCATAATTGGAAACTAGCCTGTGCTACA
TAGTAACTTTCCCCTCCCTCTTTCTTTCTCTTTGCTATGTAGCAAAGGCCTCCTTTGAACTTCAGCTCTTCCTACCTCTGCCTCCCCAGTGCTG
GGATTACAAGGATGTGCCACCACGCATGTTTGACACTCTGTCTCAAGGAATCAAAACTAGTTTACTTTTCAATGCTAAGAATTGAACCCAGGGC
CTTATGTGTGCTCTGCACACACAGTCACACTGCTACTGAGCCACATCTTTAACTTCTTTTTTTTTTTTTTAAGATTTATTTATTTATTATATGT
AAGTACACTGTAGCTGTCTTCAGACACTCCAGAAGAGGGTCTCAGATCTTGTTATAGATGGTTGTGAGCCACCATCTGGTTGATGCGATTTGAA
CTCCGGACCTTCGGAAGAGCAGTCGGGTGCTCTTACCCACTGAGCCGTCTCACCAGCCCAATTTCTTATTATGACAAATCACACTGTATAAAAA
TAAAAGGCCAGGCGTGGTGGCACATTCAATCCCAGCACTCAAGAGGTAGAGGCAGGCAGAACTCTGTGAGTTCGAGGCCAGCTTAGTCTACAAA
GTGAGTTCCAGGACAGCCAGGGCTACACAGAGAAACCCTGTCTTGAAAACATCAAACAAACACACAAGACCAAAAACCCAAAAAACAAAAATAA
ACATAAAAGTAGAAAATATATCATCAGGCTTGGCTTTAAAACTATTCAGTATTGGTCTGGTCTTTTTTGTTGTGAGTTATTTTGCTTGTTTGGT
TTTTGTTTTCTTTTTGACTCGGGGTCTCACTCTACAGGCTCAGCGGGCCTGGAACTCACTATAGAGATGGGGCTAACCTTGAACTTATAGAGAT
TCCTCTGTTCTTGCCTCCTGAGTGCTGCCATTAAAGGCATTTGCCACCTTGTGGGTATTTTGAGATAGGATCTTGCCTTGCAATCAAGATTGGT
CAGCAATTGTTGATCTTTCTGCTTTGAATCTTTATGTTTCTCTGAAAATGAATTTCGGTTGAGACAAGATCTTAATTCATGTATTCCAGATTAG
CCTGGAATTCTCAACCCACCTGCTTCACCCTCGGGAATGCTGGGATCACAAGTGTGCACTGTAACCATGCTTGACTTAAAAGTGGAATTAAAAA
AAAGGGGGGGCTGGCAAAACGGCTCATTGGGTAAAGGCACTTGCTGCCACACCTGACAACCTGGGTTTGATTGCCCTGTGGGTTTGATTGCCAG
GGCCCAAATGGTTGAAGGAGAGAATTGACTCCCATAGATTGTTCTCTGAACTCCAAACATACACTATGACATGTGGGTATTGTCCACCACTTGC
ACCATCCAAGAAAAGAAAAGTAAAAATAAGAAAAACAACAACAAAACAAAACAAATAAAAAACAAAACAAAACAAAAAAATCAACCAGGGTTGA
AGAGATGGCTCAGTCAGTGAAGTGCTTGTTTTGCCAACGTGAGGACCTGGCATGAGGCAGAGACTGGCCGGTGGATCTGTGTAAATTCAAAGCC
AGGTCTACAGAGTGAATTCCAGGACAGCCAGAGCTATAAAGAGAAACTCCATCTCAAAAAACAAACAAACAAACAAAACAAAAACAAAAAATAA
AAAAATAGCCAATTACCTAGGTATGGTGGTGTTCGTAATACCTGGCCAGCCAGCCTAGAGTAATCAGAGACCTTTAGGCCAATGAAAGACCCTT
GTGTTTAGATAGATAGATAGATAGATAGATAGATAGATAGATAGATAGATAGATAGAGTAGACAGACAGACAGACAGTGCCTGAGAAATTATAT
CTGAGTTGTCTTCTGGCTTCTACATTGACATGGGCACACATACATACGTGCAAGTGCACACACACACACACATGAATAAATAATTTAAAAAATC
CAATTAGCACACCTAAAAAAGAAATTCCTAAATGCCTTCTGAGTGTTTCATAAGAGTGTGCTAGCATCATGTGTGTGTGTGTGGGGGGGGCAAT
CCTCATATTTTGGATCCTAAGGCCACACTGCATGACATGGGGTCCCTGTTGGATGGGGCAGTGACTCCCCATCATTTCCTTTGATGCCTCTCTT
CAGATCAGATCCCAGGGGTCCCATCCTGGAAAGCAGACAGGGAATACCACATCAGTTCAGCCTTACCAAGCCCTGTGGCCTCTGTATCTTCCAA
GTTTGAGCTCCAGCTATCTGGGAGGTCCTGGCTGTTTCCTTTTGCTGAGAGTTTTGGGCTCAGAGCAGAGTCCTTTCAGTCCTCCTGCTTTGGC
AGGCTGGCCTGTCCCTCTGTAGGTTTCAGTGACCTGTAAGTCACTACCACATCACCCACGGCTTTCTTTCTTTTTGAAAAATTCTTCCATCCCA
GACCTAGCCACAGCCCCTCAGCCGTTGGTCCTGGGCTGACTCAGTCTGTCCCTGGTCCCTCCTCTCCCACCCACCCTGGGCTCACCCACTAGTA
TGGATCTGTGGTAGCGATGCTTTTGGTTCTGAGTGAAGAACAGGGGGATGACGGCTGATGAGGGCGGTTCTAGTGGGGCTCAGAGAGGGAAATG
GAGCACAAGAAGAGTTTGAGGTGAGCCAGAGAAATGGCTCAGAAGTGAGGGTGCTTCCTGTAATCCTGACAAACTGAGTTCAGATCCCCCCACA
TACTCGTGCGGAGTAGGTTACCCCAACACGCAGGCATACACCTCCCTACAGAATAAAAATAATAAAATGTAATACTTGTCTTTTTCTTTGGTTC
CCCCTACCAGGGATTTTGAGACAGGCTGCCCTGGAACTCTCTCTGTAGACCAGGGTGGCCTTGAACTCAGACATCTGCCTGTCTCTGCCTCCCA
AGTGATGGGATTAAAGGCATGTGCCACCACCTTTCTATTAGATATAATACCTTAAAAACAAACAAACAAACAAACAAACAAACAAACAAACAGG
AAGAAGAGTCTGGAGTCTCCTTGGTGCACTTTTGGAGCAACCCGGGAGCACTGCGTGCAGGCTGAGCCAGTCAGTGTGTAGTGAATGGTAAATA
CAAATAATAGCCGTAGGACGAAAGGCTTTCTCAGTAGGCAAGAGAGGCCGGTCCCCGTGGGCGTGTGGGTGGGTACTAATTGCTGGTAATCCCC
ACTTTTCTGGCTGTGATTGGTTGATTATTATAATTAAGATAACTTTGGGGAGAGCTGGTGTTAAGTAGCTCGGTTTGCAGATGCGCTTGGTTAT
GTGACGTCCGGGTGGCGAAGGCGCTTCCTACTTCATGTGAGGTAGTGCCACAGCGCCTTAGTGTCTTTTCCCCATCGTCTCAACTCTCGAAATT
TTTAATTACTTTTTTTTTTTTACTTATTATTTTACTTCTAAAATTACTTTTACTTTTTAAATATGTTTTTATGAGTATATGTGTGTGGGAGGGA
TCATGTATATGCTATGAGTATGGAGGAGCCTATAGAGATTGGAGGTCAGAGCTCCTGGAACTGGAATTATATAGGTGGTATGGAGCCTATAGAG
AAGTCTAGTACAGCAGGCGTGGTGGCGCACGCCTTTAATCCCCAGCACTCGGGAGGCAAAGGCAGGCAGATTTCTGAGTTTGAGGCCAGCCTGG
TCTACAAAGTGAGTTCCAGGACAGCCAGGGCTATACAGAGAAATCCTGTCTCGAAAAACCAAAAAAAAAAAAAAAAAAAAAGATGTCAATTATG
GGTCCTCTGTAAGGGCAGAATGTATTTTTAGCCACTGAGCCATCTCTCCAGTCTTTCATTTCTTTTTCTCTTTTGGTAAGACAGAGGCTCACTC
CAGGCTAGCCTTGAGCTCCCTGTATCCAGGTTACCCTTGAATGTGCAGCACACCTTTTGCCTCAGGTTCCTAAGTGCTGAGATTTTAAGTGTAA
GACACTGTATCCCACTAAGCTTTCAGACTTTTTTTTTTTAATTTAGATTTATTTTATTTGTAAATCTAATGTTTGGAATTATAAAATCGAATGT
TTAATAATTACTCATCTAATGAATGAATGCTTTGCTCATGTGTATGTATGTCTGTACACAACATGCATGTCTGGTGTGCTCAGAAGTCAGAAGA
TGATGTTGAAGGCAGAGTTATAGGTAGTTGTGAGCCACCATGTGTGTGGGCTCTGGGTATTGAACCCAAATCCTCTGCAAGAACAAGTGCTTCT
TTTTTTTTTTTTTGTTTTGTTATGAGACAGGGTTTCTCTATGTAGCCCTGGCTGTCCTGAAACTCACTCTGTAGACCAGGCTGGCCCCGAACTC
AGAGATCCACCTGCCTCTGCCTCCTGAGTGCTGGGATTAAAGGCGTGCACCACCACACCCGGCTATAAGTGCTTCTTTAAAAACAAAAAAAAAT
ATTTATTTTTATTTTATGTACATCGGTGTTTTGCCTACATGTATGTCTGTGTTGAGTGCTGGATCCCTTGAAACAGGAGTTACAGACAGGGGTA
AGCTGCCATGTGGGTGCTTGGAATTGAAACTAGGTCCTCTTAGAAGAGCAGCCAGTGCTCTTAAGTGGTACACCATCTCTCCAGCCCCCAAAAC
AAGTGCATTTAATTGCTGAGCCACCTCTCCACCCATGGATTTTTTGTTGTTGGTGGTGCTATATTAAACCCAGGGTCTCATGGATGCTAGGCAA
TCAATGGTTTTTTTTTTAATTTTTTTTTTTATTTTTTAAGTACTTTGATGTTTTGCCTGCATGTATCTCTGTATCAGGGTGTCAGATCCCCTGG
AACGGGAGATACAGATACTTGTGAGCCGCCATGTGGGTGCTAGGAATAGAACTTGGATCTTCTGGAAGAGCAGCCAGTGCTCTTAACCCTGAGC
TGCCTCGCCAAACCCTAAACATGCACTCCTATACTGAGTTGCGACCCCAATTTAACTTCTCACATCTTTACAAAACACTTGTTTCAAGAGTCCT
CTTTCTCCTCCTCCAGAGTCTGGGCCCATGGACTAAGAATCTCAAAATCTCTTGGAAACGGCTACTCAGAAAAGCCTAGCTGTTCTTTACCTGC
TCTGAGGAGGACTCAGCTGGTCAATCCCATGGAAAGTTGATTGATGTCTGAAACTAGTCATTGCTTTGTTGTTGGAGACAGTGTCTCACTGTGT
AGCCTTGTCTGGCCTAGAACTCACTATGTTCCCTGCCATGTCCCACAGCCGTGTAAAAATCATCGCTAGGCGGGTTAGCCTTGGGGCAGCAGAG
AGTCATGGGATCTTGACTCTGTAAGTAGTTTTAAGGAGGTCAACTTGGTAGCAGATTCATCTCCACTACTGGTTTCTTCTTAGTAAAGCAACAT
GTAGATCAGGGAGCTATTTGGGGATTCTAGCTGCTGGTTATTTCAGTAGTGTGTGTGCATATGTGTGTGTGTGTGTGTGTGTGTGTATGTACAT
GTACAAGTGTGAACATTTGTGTAAAGGCCATTAGTTAACCTTGAAAGTCATTCCTCTGGAATCCTCTCTGTCTGTCTGTCTGTCTGTCTGTCTG
TCTGTCTGTCTCTCTGTGTCTCTCTTTCTCTGTCTCTCTCTCCAGAATCTCTATATGTAGGCTGGCTTCAAACTCACAGTGATCTGCCTCTGCC
TCTCAAATTCTGGGATTAAAGGCATGCGCCAGCATGCTTGGTTCATTTATTAATTAATTTGTTTGTTTGTTTATTTTTGAGACAGGGTCTCATT
AGCCTAGAGTCTACCAAGAAGGCTAGGCTGGTTGACAAGAATCTAAATGTCTCTGCTTCCCCAGTTTTGGAATTCTAAGTAGATGTCACCACAC
CAGATGTCACCACACCAGGCTGCCTCTTCCTCTTCTCCTCCTCCTCCTCTTCAGTGTGGGCACTGGGTACTCATGTCTTCGTGATTGTGCAGCA
TCTCGCCATCTTTTAAAGTGAAACTAAGACCCCCCCTCCTTTGCCTTTCACTTACAATCTTCCTGTATCTTTTGTCACCCACTATCGGGACAAC
AGTAGGACAGATGGACAGAAACCGTAGTGTGGGGTATTCATCAATCTCTCTATAAGCTTCAGTGTCTTCAGGTGTAGAGTTTCAAGACGTTTTG
TTTCTCCAAGGACTGTCTTGATGAGTTGGCTTGTGCGTAAGTGGAGGATGGATAAACATAGTATGAGGCTGGGTATGGGGCTTAGTGGCAGGGC
CCTAGCCTAACATCCGCGAGGCCCTGGGTTCTATATTTGGCACCAAAAAGGTGTTTGCTATTCTGTTCCTTTAGAATAGAAAATATGAATGAGA
CCTGGTCACTAGGTAAGAAAATATATATGTTTTATGTTTTTTTATGTGTATGAATGTTACGTCTTCATGCATGTGTTACAGGTAGTTGTGGCCC
ATCATGTGGGTGTGGAGAATCAAGCCCAGGTCGTCTGGGAAAGCAGAGCCATCTCTCCAGCCACCTTACCCTCTTAAGACAGAGTCTCCCTGTC
CCGTAGCTCTAGCTACCCTAGAACTGCATACGTAGACCAGGCTGGCCTCAGACTCATGGAGATCCATTTGCCTTTCCCTCCTGAGTGCTGGGAT
TAAAGACAAGTGCCAACCCAGGCATGCTGCGTGGACCTCTGTCGCTCAGAGGCCAGCCTCGCTCACATAGCAAATTCTAGCATAGCCAGTAATA
CATATGAAACCCTGCTTCAAAATAATAAAATACGCTAGCCAGTGGTGGCGCACACTTTTAATCCCAGCACTTGGGAGGAGAGGCAAGCAGATTT
CTGAGTTCGAGACCAGTCTGATCTACAGAGTGAGTTCCAGGACAGCCAGGGCTACTCAGAGAAACCCTGTCTCGAAAAACCAAATAAATAAATA
AATAAATAAATAAATAAATAAATAAATAATAAATAAGCAAGCAAGCTAGGCAGTGGTGACACACACCTTTAATCCCAGCATTTGGGAGGTAGAC
CCAGGTGGATCTCTAAGTTTGACACCAGTCTGGTCTACATAACAAGTTCCAGGACAGCCAGGGTTACACAGACAAACCTTGTCTTGAAAAAGAA
ACAGGGCAGGAGAGGTGTCTAATGGCTAAGAGTACTGACTGGCTGCTTTCCCAGGGGTCCTCATTTCAACTCTCAGCAACCACATGGTGGTTCA
TGACCATCTATAGAGAGATCTGATGCCCTTTTCTGGCCTGCAGGTGAACATGCAGCAGAGCACTCATATATAAAATAAAATAAAATAAAATAAA
GTCTAAAATGTTTTTTAAAAGCAACAAATATTAATAAAGACATGCACCACCATGCCCAACTCCCTATTTTTTAAACTTATTTTTATCTACATGT
ATGTGTGCTTTTGTGAGTTTATGTGCACCGTATGTGTGCAGGTTCTTGTGTAGGCCAGGGCCTGTTGAATCTCCTGTAGAGTTACAGACAACTT
GTGAGCTGCCTGGTGTGGTTCTGGGAACCAAACCCTGATCTACAGAAGAGCATTAAGCGCTTCAATTTCAGAAACTACAATCTGAATGGCTTAA
AGTGAATGGGTGAGGGAAATGGATTGAAAATATTCTCCCCACCTCGGGTTTTCCATACTTAGCTCTTGGTCTAGGGATGTAGCAAAGCTGGTAG
GATTCTTGTCTAAAATTCACTATGTCTTAGGTGGATCCCTAACACTACACAAACTGGGTATAGTGGTGTGCACCCTAATCCTTGCACTTTGGAA
GTTGAGGCAGGAAAATTCAGTCATCCTTAGCTACATTCTCAGTTTGAGGCTCCTCTAGTCTATAGGTTTCTTGATTAAGAGACAAAAGCTTTCC
TATCCCATGTCGCTCCCTGAGAGGTGGGATGTGGGACCTAAAGGGATGTCCAGTGAGTGTTTGAATCTGTTGTTCTGACCACACCGCTCTCCCC
GCCCCCTCCCCACTCCCAGCTGGGGCACAAAGGCAGAGCTCAGTCATTGGAACTGGTGATGCAGAGACTGGGCATGGTGGCTGTGGCAGTGAAG
GCTGTGGCTAGAGTGGACAGAAGAGATGGTGATGGTAGCGCTTGGAGACAAGACGGCTGTGATGGTGATAATGAAAGTCAAGACTAGCGTAATG
GCAACTGTGAGTCAGTGGCTGTAACTATGATGGAGATGGTGGGTGTGGTGACGGGAGTGACGATCGCCATGGTCATGGCGGAGGCGTAGCCAGA
GTGAGGGTGGGAGTGGTGGCTTTGACGGAGGTGGTGGTTTGAGAGATAGAAGACATGGTGAAATCGATGGATATATTAATAAGATTGTAGAGAT
GGTAGCTGCAGATGTGCTGGAGGTGGTGTCTGTAGACATGGAGGTAGCGGTGTCTGTATTATGGTGGACATGGTGGCTGTATTGGTGGTGGTGG
TGGGGTGGACGTGGTACTGGAGACAACATAGCTGTAATCATGGTAATGGCAACGGCAATGATTGTGGAAGTGATAGTAACAACCCTGGAAGTTG
TACTGGTGGAGACGGTGTCACCGTGATGGTGGAGATAGCGGTGAGAATGATAGTGGTTGTGATATGGAGGTGGTGGTTGTCGTGATCGTGATAA
TGGGGGGCAGCCATGGTGGCGGTGGTGGGCAGGCCTTTGCAGGACAGCGCTCCCTTTTCTGATGCTTTCACTTTAGAACAAGGGAGCGTCACGC
AGGGTCCCACCCACCCACCAACCTGGGACCCAAGACTAAGCATAAGAACGATTAGAGCCTTCAGGATGCCTAGGGTGTTTGATGAGTTTCTGAA
GGCCAGACTTGGGGCAGGGCACACTATAGATGTGACTGGAGTGTGGGACAGGGAGGACCTGTCAGTGACTTCCATGGGGCAGGAGTGTGTTGCA
GAGCGGGAGAGTCATCCTGGAGCTGTGGAACTCAGGCACACCCACCTTCTGGGACTATCTTTAAAGGGTTGCCTGGTGCCTTTGGCCCATGCAG
GAAGCTCACTAATGTTAGAGACAGTGGTTTCCAGATAATTATCTAGTAACTGATCTTTTGAGAGGAAAGCGTTATCAAAAGTCTGCAGGCTGGG
ATGTGGCTCAGTGCTAGACTGCTTGCCCATCATGCATTAACCCTTGGGTTTAATCAGCACCACTACAGAGGGGTAGGCACTGATTTACTGTCTC
TGCTGGTGCCCTGCAGCTGACTCTAAGCTACCAACATGACAGGGTTGAACACAGAATTGGGGGCAATGCACAGTAGCCATTGTCTCTGTTCTTC
AGCTGCCTCAGACAAAGACACAAGTAAGCTCAAGCCGGGGCCACAGTACAACACGTCAGAGCACTCAGAAGAGACTCTTGGGTTGTGTGTACCT
TGGCTTTTGGTAGAACGTGATTATTGTTAAGTGTGAACAATTTAATTTTGTTTTTGTTTTTGTTTTGTTTTTCAGGACAGGGTATCTCTGTGTA
GTCCTGGCTATCCTGAGACTTGGTTTTATAGACCAGGCTGCCCTCAAACTTCAGCGATCCTCCTGCCTACTGCTCTTAGGTGCTAAGATTAAAG
ATCTGTTCTACCACACTGCCTAGTGCAATTTAACTTTAAAAAAAAGATGTATTGCTGGGCGTGGTGGCGCACGCCTTTGATCCCAGCATTTGGG
AGGCAGAGGCAGGTGGATTTCTGACTTCGAGGCCAGCCTGGTCTACAAAGTGAGCTCCAGGACAGCCAGCGCTACACAGAGAAACTCTGTCTCA
AAAAAAAAAAACATATATTATTAATTTTGTGTGTGTGTGTGTGTGTGTGTGTGTGTGTGTATGGACATGTGGGTGTCAGTGGAATCCAGAGGCA
TTGGATCACCTTGGAGCTGGAGTTAAAGACTGTTGTAAGCTGACGTGGGTGCTGAGAACTGAACTCAGTCCCCCTGGAAGAACACTATATGCTC
TTAACCATTGAGCCATTTCTTCAGTCCTATGAATTAATTTTCTGTTACACACACACAGACACACACACGTATATTTAAATTTATTTATTTATTT
ATTTCATATATGATTACACTGCAGCTGTCTTCAGACACACCATAAGAGGACTACAGATCCCATTACAGATGGTTGTGAGCCACCATGTGGTTGC
TGGGAATTGAACTCATGCTCTCCGGAAGAGCAGTCAGTGCTCTTAACCGCTGAGCCATCTCTCCAGCCTACATCTATATTTAATAACTAACAAG
CTAGCTACCTTCTGACACACTTGTCAGTTGGCTCTTGAGGACAAGTGAAGGCCGGCTACAGCTTGACACATGCACACAGGTGACTGCCACTGAG
CCCTGGCTGATGGTCCCTGGAACTTCTCTGTTCCCCACAGGCGAATGTTCCCATTCCTGTCCTTCACCGTGGCCGGGCTGGAGCCCACAAGCCA
TTACAGGATGTTTGTGGATGTGGTCTTGGTGGACCAGCACCACTGGCGGTACCAGAGCGGCAAGTGGGTGCAGTGTGGAAAGGCAGAAGGCAGC
ATGCCAGGTACAAGCTTCGGGAGTGGTGGGCATGCGTTTCCTCCTAGACAGGGCGCCCCCTGGTGGATTACTCAAGCCCTTACCCCGATGTGAA
GGATTGAACCCTACAGCCTATCCACCCACCTCACCCTTCCTTTGGCTTCTTACCTCTTCTCTAATTTGATTTAACTGAGGTGGGGAGTGGGGGA
GGGAGAAAGAGACTCACTCTGCTCAGTCCTCTGCCCTTATCTGCCCCTGCTCAGCCTGTCTGTCCTCTCCCTTGCATCTTTCTCACGGTCCACT
CAGAAGAGGCAAAATCCCACATCTCCAGTGTCTGGTCGATACTTGACAATAAGGTTAACTGTCCACAGGGAACCGCTTATATGTCCACCCAGAC
TCCCCCAACACCGGAGCCCACTGGATGCGCCAGGAAGTTTCATTTGGGAAGCTAAAGCTCACCAACAACAAGGGGGCTTCCAACAATGTGACCC
AGGTAGGATTGTCAGACCCACCTGCCTATATGTCTGAGATCTACCCCCTGACATTTCCTAAGACCCACTTTGTACTGTTGGAGTCTCGTCTCCT
CTCCTCTCCTCTTCTCCTCTCTTCCCTACATAGCCAAGGATAACCATGAACTTCTGTTCCATCTGCTCCTGAGGGTGAGATCACAGGCATGCAC
CACCAGGCCTGATTGATGTGGTTCAATCAGATATTGCACCAGACTAGACAAAGGTGCTAGCAATTGAGCTGCTCTATGCCCCGGCTCCTTTTCT
TCCTCTCTATGAGTCTTTCCTACCCATCTTTGGGAGACAATGAGATGGGTGGACACTGGATCTCTGGCAGTGGTTGCTCCTGGGTCAACACAAG
TCAGAACAACATGAGGGCTTCTTCACTAGATCATGATCTGTCACCGAGATGGTCCTAGAGCTTCAGGGAATGGATGTTCAGTGTGAAAGTGACT
TTACATTGAACAATTATGGGTCTTGTTTTGGTTTGTTTTACACATTTACTTCATCCATCCATCCATCCATCCATCCATCCATCCATCCATCCAT
CCATTCATCCATCTATCCATCCATCCATCTATCCATCCATCCATCCATCCATCTATCTATCTATCTATCTATCTATCTATCTATCTATCTATCT
ATCTATCTATCTATCATCTATCTACTTATGTACTTATTTACTTAGTAAGTTACTCAGTTAGTTAGTTGTGGGGGAGTAAGTTGCTATGATGTGT
TTGTGGAGGTCAGAGGACAACTTGCTGAAGTTGATTCCCTTTTTCTACCACGTGGATCCCAGGGATCAAACTCAGGTCGCCAGGCTTGGCAGCA
AATGCCTTTATCTGATGAGCCATCTCCACAGACCCAGTCCTGTTTTGTTTTTTTGTTTTTTGTTTTTTTAAATTAAACAACAAAACAAAACCTG
GAGCTGGAGAGAAGATTCAGTTGGTAAAGTGTTTCCCTTGAAAGACAAAGACCTGAGTTTCATCCCTAGAACCTAGGTTAAAAGAGAAAAGAAA
AGCCAGTTGTAATGGCATGAACCAGTTATTTAAACACTGGGAGCGGGTGGCGGGGACACAGGTGGATCCCTAGGGCTCACTGGCCAGCCAGCCT
ACTTGGTGAGTTCCAGTAGGGATGCTTAGGGGAAGTTGCATGGTGGTAAGGTGGTCGAAGCCACAAGATGAACTCAGGCATCCTTTCTAGTCTG
TCTCACTGTGGGGCTAGTAGTTACGTAGGAGAGTGCATCTAGAGATGAGGCCAGCTTGAGGTCAGCAGGACCTTCCTTCCAGCTCCTTCCGTGA
CCCTGTCACCCCCTGGCCCCTCTCCAGATGATCGTCCTGCAGTCTCTCCACAAGTACCAGCCCCGGCTGCACATCGTGGAGGTGAATGATGGAG
AGCCAGAGGCTGCCTGCAGTGCTTCTAACACACACGTCTTTACTTTCCAAGAGACCCAGTTCATTGCAGTGACTGCCTACCAGAACGCAGAGGT
GAGCCCGGGGACGGGCATCTGCCAGAGGGAGGGTGGGACAGACTTCTTGAGGAGGAAGAGGGGACTCAGAGAACTCTATTTCCCCGTCTCTAGA
TCACTCAGCTGAAAATCGACAACAACCCCTTTGCCAAAGGATTCCGGGACAACTTTGAGTCGTAACTGTTCTGAGTTCAAATCCCCTTTGGTCT
TTCTGAGATGGGACTGTGGTTCAGTGCTCAACATGCCCTGTCTCTGCTTCAGGGGTCTGGGGACCATCATATTAGAAGCCTAATATAGCCTTGA
TCTCTCTTTCTCTCTCTCTGTCTCTCTCTGTCTCTCTCTGTCTCTGTCTCTCTGTATTTATTTTATTTATACGAGTACACAGTAGCTGTCTTCA
GACACACCAGAAGAGGGCATTGGATCTCATTACAGACGGCTGTGAGCCACCATGTGGTTGTTGGGAATTGAACTCAGGACCTCTGGAAGAGCAA
GCAGCCAGTGCTCTTAACTGCTGAGCCATCTCTCTAGCCCCCTCATTCATTGTTTTTATTTTTATTTATTTTTTAGCATGTACGCATCTGTTGA
TACGAGTGTCCCCTCGCCACCTGGACCCAACTGTCAACTGCTTGGGGGAGACCCCTTCTCACCTCTTCTATCCAACCAGTATCCTGTTCCCAGC
CGTTTCTACCCCGACCTTCCAGGCCAGCCCAAGGATATGATCTCACAGCCTTACTGGCTGGGGACACCTCGGGAACACAGTTATGAAGCGGACT
TCCGAGCTGTGAGCATCAAGCCCACACTCCTACCCTCTGCCCCGGGGCCCACTGTGCCCTACTACCGGGGCCAACACGTCCTGGCGCCTGGAGC
TGGTTGGCCCCTGGCCCCTCAATACCCGCCCAAGATGAGCCCACCTGCCTGGTTCCGGCCCATGCGAACTCTGCCCATGGACCCGGGCCTGGGA
TCCTCAGAGGAACAGGGCTCCTCCCCCTCGCTGTGGCCTGAGGTCACCTCCCTCCAGCCGGAGCCCAGCGACTCAGGACTAGGCGAAGGAGACA
CTAAGAGGAGGAGGATATCCCCCTATCCTTCCAGTGGCGACAGCTCCTCTCCCGCTGGGCCCCCTTCTCCTTTTGATAAGGAAACCCAACGCCA
GTTTTATAATTATTTTCCCAACTGAGAAAATGCCGCTGAATTGGAAGGTGCCCACTAACTTAGAAAACAGACGCGGGGCTGAGAGCCCCGAGCT
CTTCCCCATCCCTTCCCTGTATAGTGATTGGTTGGAGAGGAAGCGGGGCAAGAAGGATTCTGGGGTTTACTTCTTGTTTCCTGGCCCACAAGGA
AATACGACAGGAGTGTCCCCTGCCCCTTTCTCTGCCCGAACTACAGTCACGAACCTGGTGCTGCTTCTGACCCCATGGTTCCATGGAGAACGGA
GAATGGACTCCAGAGAGTTTTGGACCCAGAGGGACTTCATGGCTTTCTGCGAGGTGGAGGGGTCGGGGTGGGGAGTCCAGGAGAGCTGCTCTCT
TCCCCTGTCCAGTCAGTAACTTTCAACTCTTCGTCTGACACCTGTGTTAATCTCTGACCTGAAAGTGAAGATACACGCATTTTTACAACAGCCA
GCCAAACAGAGAAGACTCAGGTGACTGCGGGCGGACTGGGCCACCTGCGAGGAGACAAGAGAGGGTGGGTGCAGAGGAAGGGTTTGAAGGGTGC
ACATTTCACCAGGCGAGGTCACTTTGAACCGGTGTGTACACACACGGGTGTCTCTTTTTTATTTCTTCGGGAGGGGGGAGGCTATTTATTGTAG
AGAGTGGTGTCTGGATGTATTTCTTCTGTTTTGCATCACTTTCTGGAAATAAACATCGACCTGGTAAACACGCCCTCCTTCTGTGGCTGGTCGG
TAGGAGCTGTGTGTGTGTGGATCAAAGTTTGCTGATCTAGCAAAGTTGTCCTGTGGGCTCTTGATTTTGGGGGGTAACTGCCAGGAACCTAAAG
TGACCCCACTTAGGGATAAACTCAGAGTATGTCTTGTCCTCAGCACTCCACCTGATCGTTCATCCCACCACATGTGACCAAAGGACCAGCCCCT
CAGCCTATGTTGCCTGCTCAGAACCCTGGAGGGAGGCAGTGTGGGAGCAAGGTTAGGACCAGGCTGCGTCCGGTGGAGTTTAGGAAAGCCAGGA
CTCTTGATTTATCTTTGCATCACTCTCCATTTTTTAGGAGTTTGTTCAAGTGTTAAAGGCATAAGTAGAAATTATACCATGTGATGTTGCCACT
CTTTTGTAGGTCACGATCAGGCATACACTAGCAATTCCACATGAGTCACCTTAGTACACACTCGGCCAGCATTAGCCCTTCATGAAGTCCTCAG
TGTGGGAGCTGTCATTGTGAACTGGTGTGCACTCTCCTCTCTCTTTTCCCCCCACCCCTCTTATTTAGTTTTCATTCATAACCATAAACTTAAC
TCTGAAATCCAGCTAGACTTGGAGGGGAAGAAGGGAAATATGGGACCCCAGGCACTTCAGTGAGAGCAGAGATTACAGGGTGTTGCGAGCAGAT
GACGTGGGGGTGCTTTCCTGAGCTACAGAAGAAATGGTCTCGTCGGTAAGATGCTCGAAAGTCAAAAGAATTAGAGTTGTCCATGCTTGGAAGT
GGGGATCTTATTTTCCTCCTGGTTTTGCCATTCCTGGACCCCCAAACATTCCATGGCTTCCACGATGTTCATGCCTTCTCTCCCCTTCCCCTCA
GTCTTGCCAATGCAGGTAAAAAACCAGTGGGAAGCATTAATGTTTGGTCCAGCATTTGCATGGACAAGATGCCAATGACCGGGGTGCTTCAGGA
TGAAGTTCTCATGCTCAAACTTCTCCCTGTAGATGGACCTGCCGCCAGTGCCACGGTGGTGTGTGACATCACCACCTTGGCACATGAATCCTAG
AATTACTCTGTGAGAGTAGGAACCCTTACACCCAAATCCTATCTCTCCAGACAGAGCTCAGAGCACGGAAGTTCTCTGCTGCCTTTGGAACTGT
GTCTACAAACAGCTTCAAGATTATGTTGTAACCCAGGCTGGCTTCCAATTCACGGTGATCCTCCCATCTCATCCTCCCAAGAGCTAGGATTACA
GACACAAACCACCATGCCCAGCTTTCTAGCTCTCTCTTTCTGACAGAGAGAGAGAGAGACACAGAGAGAGAGAGAGAGAGAGAATTTGAGTCAG
GGGTAAATGGCTAGTTGCACAGACTGACCACCCAAGCTTGATGCCTTGAATTCATGCAACAGCTGAATCTGGTAGCACACATCTGCCATCCCAG
AGCTCCTAAGAGATGGGAGGCAGAGACAGGAGGGTGTGCTTGGCAGCTTACCAGATGACTGGCACAGTAGGACAGTAGGAAAACACTGGGGAAG
AGAACAGTTTTAACTTGGCAAGAAGAGAACCAACCTCAAAAGATTGCTCTGATTTCCAGGTTGGCCTTACAACATACACCAGGCTGGCCTCAAA
CTTACAGAGATCCACCTGCCTCTGCTTCCTGAGAGCTGGGATCAAAGATGTGCCCCACCACACCCAGCTGTGTATGTGTATATTTCTGTGGGTG
CCATGCAAACCACAAGTGGGTATCACATTCCCTGAAGCTGCAGTCAGATGTGCTTTTGACCCTCCAGATCAGTCATAGTTTGGTGGGAACCAAA
CTCAGGCCCTCTGTGACATCATCAGATGCTCTCAACTGATGAGCCACTGCTCCACACCCTAAAATTAAAAAAACAAAACAATTCAAGCCAGTTG
AGGTGGCAAGTGCCTGCAAACTCAGCACTCGGGTGACTGAGACAGGAAGGCAGACAGTTGGAGACCAACCCAGGCTGCACAGTGAGACCCTATA
GTGAGGAAAAGAATAAGGACAACTGCAAATGATATCAAATTTTGCTACACTTGAATAACTGACATTACCATTCAACGGGTTAGACAAACAGAAA
TGGTTTATAAAATTGTGACCCTACAGAAATCACGTTTTTGTCTTAATGAAAATGATTNNNNNNNNNNNNNNNNNNNNAAAAAAAATAGTCTTTG
AAAGGAACGCTGTAAGGTTAAGAAAGGTTTTTTGAGCTAGAGTTATAGATAGTTGTGAGCCACTATGTGGATGCTGGGAATTCAACCTGGGCCC
TCTGCAGGAGCAACCAGTGCTCTTAACCACTAAGCCATCTTTCCAGTGCATTAAATTCTTTTGTATTCATTTACTTGCTCATTTTTATACGGAT
TAGTGTTTTGCCTGCATCTATGGCTGTACACTTGGGTAGAGTGCCTGATGCCCACAGAGGTCAGAAGAGGGTGTCAGATGCCCTGAGACTGGAA
TTTCAGAGGGTTGTGAGCCACCATGTGGGTGTTGGGAATTGAACCTAGGACTCTCCAGAAGAGCAACCACTGCTTTACCCACTGACCTATCTAG
CTCCACACCACAAACTCCTAAGGCATTTCTCCACTATGTTTTAGTCGATACCATCTCTCTTAGGAATTATGATGCTATTTTGATTCATGATCAT
CCACTTTAAATGTAGGTGTTTCTTTCATCTTCTGTGTTCTGAATATTGAATGGCACATGTTCGTTTGAAGTCTTGTGGCAGGCTGGGAGTGGGT
ATGTGCATGTGGGTGGATGTTACTGAGGCTCAAACCCAGGGCCTCGGGTGTGCTAGGCAGGCACCCCACCAGAGAGCTACATTTCCAGTCTCAT
CGATGTTAAACCAGGGGTTCTCAACCTGTGGGTCTCGACCCCCCCCTTCCAAAGCACTGATGATTACGTTGCCATTCAAAGTTATAGTCATGAA
GCAACAAGATGATTTTGTGTGTGTGTGAGGGCAGTTCTCACCACAGCATGAGGAATTCTACTAAAGAGTTGCAGCATTAGGAAGGTGCAGAATC
AGGGCTCTGGATTCTCTTTCACATTGAAGTCTTAAATACTGGGTGATCCTTATCAACTCACATTTAGTAGCAAGGCTCTGGTAGTCCCTTTAAG
AGCACTTGCGTGTATAGGAGAAGTGGCTATCTCTCACCGGCTTCCCATAGGGTGATCCCGGAGGCTGACCCTGCAGAGAAGCTGGCAGTGGGGT
GCTGGATCTGACCTCCATTAGCTTATGAAGCCTGAATCTATTGAATCCGGTGGCACCTTTAATCCCAGAGGCAGTTGGCTCTCTGTGAGTTCAA
GACCAGCCTGGTCTACATAAGTGAGTTTCAGGACAGCTAGGGCTATATATTAGAGAAACCCTGTCTTAAAGAACAACAACAACAAAAGACCCTC
AGGCCACACTCGCATGCAATCTGTTCCTTAAGACAAAGAACTAACAGGTGCCAGCCATACCTGTTAAGAGTCCTTAACTCTGAGGGAAAAGCCA
GCCCAGCTTCACCCTCCCTCCCTTTCTCTCTTTCTCTCTCTCTCTCTCTCTTTCTCTCTCTCTCTCCTCTCTCTCTCTCTCTCTCTCTTTCTCT
CTCTCTCTCCTCTCTCTCTCTCTCTCTCTCTCTCTCTCTCTCTCTCTCTCTCTCTGGGACAGAGTTGATGAGAAATACTGGACTGTGGTCTTAC
AGTTCCACCTTATAGTCCTGTCTAGTGCCTTTTCTGTGCCCCTTGACAGGTAGGCAAGGTTAGACCATGTAATATGAAGCTTGCCCACTGAAAA
CCTGTAAGTGTCAGAAACTGCTCTCTTCATCTGCATTTGAAGGAATACCTCTCCTCTTTCCACCTTTAAGGATCCATTATGGCTGTGATAGTGT
AACTGATAGTCTGTGAGCTCAAGGGTCTGAGTGAGCCTTCATGTCCCTTCATGCCCCATCATACTAGACATGGTGAGTATCTCTCCTAATGAGG
CCACTGTCACTTCTCAGGGCCCCATGTCATGAAGCTGTTGTGAAGGGTGACCTCATCTCGGCTCCTTTCTATGAATCTGTCCATTCTCTCTTAA
GTTGAAACAGAACAACTATAGCTTTCCCTCACGGCATCCGGTGGCATCAGGTTGGTAGCTTGACATCGCCCATAGTGGGAATATTTACACTATG
GGAATAGATCCGTGCTGCAGTAAGGCACAGCCCCTCCCAAAGAGCTTGTTTCCTAACGCAAACTTGAGAGTTGGCATCCCCTGCAATATGAGTG
CACGGAGAGACCCCTCCTGAGAGGATCCATTCTCCACAGGAAAGCCCCCAGCAGCTGACAGCGCCCTGTGAGTGGGGCAGGAGAATGGAAAAAA
TGGAAGGTCACTTTTTTCACCCCCAATTTCTATGTGTTTATGGTGTGTGTGTGCATCCATGCACACGTGTGTATGTGAGTGTGTGCACGTGCAT
GTGCTATGGCATGTGCACTGACGTCAGGGGACATCCTTGGGTATCAGCCCTCCCCTCCCACCTTGTTTGAGACTCTATTATTTTTCTACCCTGT
GTATAAATTGCTGGCTTGTGAGCTTTTAGGGATTCTCCTGTCTTCATCTATCATCTCCTGGGAGCACAGGGATTACAGGTGTGTGAACTGCGTG
CCAAGCTTCTCCATGAGTGCCCCGCACATAAACAAAGCTGTATCACCTTCTATGACAACCGCTCTTACTCACTGAGCCATCTCTCTTGACCCTC
TGTGACAAGTGTGACTTTGATTTTCTTTTTTCCATTTTGCCAAACCGCTCTTACTACCTTTAGGCTCCCAAGTCCTGCCTTTCTCCTCCTCTCC
AGAGAAGGACTCTCTCCTTCTAGGTGGGGATGCTGGTGAGACGGCTCTGGGCGCTCAGCGTGAGAAGCAGACATGAGCATCTTGTCATGTCTGA
ATTCAGACTTCTGATCATCTACTAGTTCTCTGCAAGGGGCTCAACCCCCTCCTTCCACCGCAGCTGACTGCCCCTTGGGGGATGAATCAGCTCC
CTCGTTGCTAGGCCTTTTAGAAGACATGCTAATTCTCTGCTCCATTTTGTGCAATGGTGTTATTCCCAAGTCATTCTCAGCCACATAGAAAGCT
AGAGGCCAGCACAGGCTCCATAAGACCATGTCTCAAAATAATCTGTCAGATCATAGTCCTTATTTTTAAAAATTCTGTCCCCAACCTCTCCCTC
AGTAGGTCAGGTTTATATGATGGGAAGGGGTCTCCTCTTGTGCCTCACCTTATACCTTATCAGAGCTTGGTGAAAGGGGCTTGTTACATGGAGG
AGATGGGATTGAGAGTAGCTAGCAGATTCGGGTGCCCTTCAAATGGAGGCTGCGTGAACTGTGTTGTAATGGAGCTGTCAGGGAGATGGCCATG
TGGGAGGGGTGGGGTCCTTAGTGCTGTGTTGCTATGAGGATACAAGCCATGACCAAAAGCACGTGGGGTGGGGGGAGAAGTTTTTATATTTAGT
TTACACATTCTGATCTTGGTCTATTATTGAGGAAATATAAGGCAGGAGCTGAAGCGGGGACCATGGAACAATGCTGCTNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNTCTCTCTCTCTCTCTCTCTCTCTCTCTCTCTCTCTCTCTCTCTCTCTCTCTCTCTTTCTAGC
ACAGAATTGTCTTCCCAGAAGTGGCACCACTCAGAGTGGACTATGCTCTCTCACAACAATCAGACTTGCCTACAGGTCAATCAAGTGGAGGCAG
TCCCTCAGTTGAGATGCCCACTTCCCAAATGACTGGAGTTTATGTCAAGTAGACAAAAATAAAATAAAATTAAATTAAATTAAAGCAAATTAAA
TTAAAGTAAAATAAAAATAAAATAAAATATAAACACTCTACTAGGCATCTATACTCTCATTTCTCTGGAATCTGAGGCAAGAGAGTAGCCATGA
ACTCAAAGCAAGCCCAACCTATTGAGATGAGCGTGAACCTATCTCAGTGCACAGACACAGCAACACACTGTTAGTCTTGGTGCCCCCAGCAAGA
GCACAGGAAGAAATTCCCTGGTCCCAGCTCCTCTGAGGTCGTCCTGATGCTCCCTGCGTCCTGCAGCCACTGCAGGTGCTGGCGGCAGCGTCCC
TTTGGTTTGGAGTGGGCGTCTCAAAGTCTTCAGCACATGTCACTGAGGATATCCTCGCCCAGTCATGGTAACCAGTCTCTGTGAGCAAGAGGGC
AAAGACGCGATGTACGCACTTCCTTTTATGGGAAGATGCTACCCAGCTCTTCCCACACCAATATCGGCTACCCAGGCTGGGGGTGGGATGGAGT
CAGAGACCACATCTCACCCCTGTGGACATCATTTTCAGATCAAGAAGGTGGTGTTGTCGTTCCTAGATCTTGCTGAAGTTTTTGTTCTCCTTTT
CCTTTTGAGACAGCCTTTCCTGCAGAGTTAAGGAGACAGTTCTCTGAGACAGGGTTTCTCTATGTAGTCTTGGCTGTCCTGAAACTCACTCTGT
AGACCAGGCTGGCCTCGATCTGTGTGAAAGCAAAGCTTGTGGAGTTTGCTACTGTATACTCCTAACCATGGAGTTAAAGTCAGAAAGATGGCTA
GTGATTTTTTTAAAAATATTTATTTGTTTATTTCATGTATGTGAATACACTGTTGCTGTCCTCAGACACACTAGAAGAGGGCATCTAATCCCAT
TACAGATGGTTCTGAGCCACCATGTGGTTGCTGGGAATTGAACTCAGGACCTCTGGAAGAGCAGTCAGTGCTCTTAACCGCTGAGCCATCTCTC
CAGCCCCCGTGATGTTGTTCTTCTTGAGGATGCAGGTCCTATTCCCATCACCTATCTAGCATCTCATAATCAACTGTAACTCCAGTCCCCGGGT
ATTCGATCCCCTCTTGTGGTCTCTGTATGCACAGATATACATCCACACAAAACACCCATACACATAAAAATAATATAATTTAATTTCTTATTAT
AAAAACTTTCGGAGCTGCTATGAGTTCAAGTGTGACCTGCTCTTCACATGGAATTCTGGGCAGGCCAGGGCTATGTAGTGAAAACCTGTCTCAA
GAAACAAACATTACAGACTAGGTCCATATCTTATGGTGCTCAGAAAATGACTTGTGGTTCATTCTGATTGATCTGCCACACATCGGCAGTCAAT
TTGGCTGTGGAGTTGGAAGCTGACTGATGGGCACAATCTGGGAGCAAAAATGAACCCAGATTAGTTTAATTATCCTGGGTGATGATAAAATGAG
GGAGATAGTTCTTTAAAGGAAATTCATATGAGAGTTCTTTGTCTATCTGTCCATCTGTCTGTCTGTCTCTCTATCTCTCTCTGGGTCTCTCTGT
GTGTGTNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNATTATTCACAACACTGTGTGTGTGTGTGTGTGTGTGCG
CATATATGAGTACATACAAATATAGGTATGTGTGTGTGCATATACATATTGGAGCTGCCAAAATGGCTCAGCATTTGCAACCAAATCTGATACT
GTAAGAGTAAAACGTAAAAACAAAATTCTAGGCCTTTAGGCCCAGGCTTGGGAATAAGGCCTTTGTCCTTTGTGACTCAATGTTCCAAGGTATG
CTAATCCTAAAACAGATAGCGACATCCCTCCACCCACCCACTTCCTGGGTTCAAGGAGTATTCATTCAAAGAAACTCACCCTGACCTACCCTGA
CCATTGCTGGAACCCGCTATGCAAAGGTGCTATTGTTAGGGGCTCTTAGAAACTGTCTTGAGAAATAACCCTCACAATTACCAGGTATTCCTCG
TGACTCTTGTGACTTTGCACTTCCTTGTGACTCTTAACTGGCATCTTTGGTATTTTTTTTCCAACAATGCCCTTCCCACCTCCTTGAGTTGTGG
TTTCTTCCTTTAAATACCCCCTTATCCAGCTACTCGGGGCGTCAAGGTCCTCTACCCCTGCGTGGTGTATGACCGTGGGCCTGAGAGCGCTCTT
GAATAAAAATCCTCTTGCAGTTTGCAGCAAGACCGTTTCTTGTGGGTGATTTTGGGGTGTCGCCTCTCCTGAGTCAGAACGTGGGGGAGTCCTC
ACGTTGTGGGTCTTTCAATACNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNTCCTTGATCCCCCAGAAGTCATGAGAAAAGCTGAGCACTGTGGGGTGCACCCCAACGCCCAGCA
CTGGGGGAGTGAGGACAGGCTGGTTCCAGAGGATGGCCCACCAGCCAACTTAACTTAATCAGGCGGAGTCTCCCTCCCTCCCCCAGGCAAGTGG
GCTGCATCTGGGGATCACACACACGCACATACACCACACATCTGTTCATAAACCCACATGCACATGAGAACACACACACGGTATGTTTGCATGC
ATTGGCTGCCTTTGAAGACGGTCGTGTTGAAATAGGAGCATCAATGCAGTGTTGGTGGAAGACGGAGCCTTTGAGCAGTGACCAAGAGGGATGA
AAGTCCTCTCACGGGAAGAGCGGGTCCTTGGGGAATGCGCCCAGTCCTCCTTGTTCTGTTTTATATTTACTCTCACCATCCCACTTTCTGTCAT
GTGACAGTGCCCTAGCCACATCGTGCTGTTATGTCCGTGCACACCCACCTTCCACAACCCTAATTTAGACCAACTTCTCTCTCTAAATTACCCA
GTTGTGAATATTGGCATAACAACAGAAAACGGACTGAAAGCTAAATATCCATTTACATGAAACCAAATCACAGAAGGGAATAAAATCCTTCTTG
TTCCC
MOUSE SEQUENCE - mRNA
GCGGCCGCGTCGACCGCGCGCTCAGGAGCCAAGCGTCCCAGCTCCCGCTCCAGTGAAGTTTCATTGGTCTTCGGACGCCGCCCCCGTCGCCCCA
GCCCTAAGGACCCTCGGGTCTCTTCCACGCCTCCTCGAAGGCGCCCAGCCCGCCTCGGATGCGCATCGTGGAGCCGGGCTGCGGAGACATGCTG
ACCGGCACCGAGCCGATGCCGAGTGACGAGGGCCGGGGGCCCGGAGCGGACCAACAGCATCGTTTCTTCTATCCCGAGCCGGGCGCACAGGACC
CGACCGATCGCCGCGCAGGTAGCAGCCTGGGCACGCCCTACTCTGGGGGCGCCCTGGTGCCTGCCGCGCCGGGTCGCTTCCTTGGATCCTTCGC
CTACCCGCCCCGGGCTCAGGTCGCTGGCTTTCCCGGGCCTGGCGAGTTCTTCCCGCCGCCCGCGGGTGCGGAGGGCTACCCGCCCGTGGATGGC
TACCCTGCCCCTCACCCCCGCGCGGGGCTCTACCCAGGGCCGCGCGAGGACTACGCATTGCCCGCCCCCTTCCAGCTGTCTGGGAAGCTCACAC
TCGCCCTCAGCAACCACCTGTTGTGGTCCAAGTTCAACCAGCACCAGACAGAGATGATCATCACTAACCAAGGACGGCGAATGTTCCCATTCCT
GTCCTTCACCGTGGCTGGGCTGGAGCCCACAAGCCATTACAGGATGTTTGTGGATGTGGTCTTGGTGGACCAGCACCACTGGCGGTACCAGAGC
GGCAAGTGGGTGCAGTGTGGAAAGGCAGAAGGCAGCATGCCAGGGAACCGCTTATATGTCCACCCAGACTCCCCCAACACCGGAGCCCACTGGA
TGCGCCAGGAAGTTTCATTTGGGAAGCTAAAGCTCACCAACAACAAGGGGGCTTCCAACAATGTGACCCAGATGATCGTCCTGCAGTCTCTCCA
CAAGTACCAGCCCCGGCTGCACATCGTGGAGGTGAATGATGGAGAGCCAGAGGCTGCCTGCAGTGCTTCTAACACACACGTCTTTACTTTCCAA
GAGACCCAGTTCATTGCAGTGACTGCCTACCAGAACGCAGAGATCACTCAGCTGAAAATCGACAACAACCCCTTTGCCAAAGGATTCCGGGAGA
ACTTTGAGTCCATGTACGCATCTGTTGATACGAGTGTCCCCTCGCCACCTGGACCCAACTGTCAACTGCTTGGGGGAGACCCCTTCTCACCTCT
TCTATCCAACCAGTATCCTGTTCCCAGCCGTTTCTACCCCGACCTTCCAGGCCAGCCCAAGGATATGATCTCACAGCCTTACTGGCTGGGGACA
CCTCGGGAACACAGTTATCAACCGCAGTTCCGAGCTGTGAGCATGAAGCCCACACTCCTACCCTCTGCCCCGGGGCCCACTGTGCCCTACTACC
GGGGCCAAGACGTCCTGGCGCCTGGAGCTGGTTGGCCCGTGGCCCCTCAATACCCGCCCAAGATGAGCCCAGCTGGCTGGTTCCGGCCCATGCG
AACTCTGCCCATGGACCCGGGCCTGGGATCCTCAGAGGAACAGGGCTCCTCCCCCTCGCTGTGGCCTGAGGTCACCTCCCTCCAGCCGGAGTCC
AGCGACTCAGGACTAGGCGAAGGAGACACTAAGAGGAGGAGGATATCCCCCTATCCTTCCAGTGGCGACAGCTCCTCTCCCGCTGGGGCCCCTT
CTCCTTTTGATAAGGAAACCGAAGGCCAGTTTTATAATTATTTTCCCAACTGAGAAAATGCCGCTGAATTGGAAGGTGCCCACTAACTTAGAAA
ACAGACGCGGGGCTCACAGCCCCGAGCTCTTCCCCATCCCTTCCCTGTATAGTGATTGGTTGGAGAGGAAGCGGGGCAAGAAGCATTCTCGGGT
TTACTTCTTGTTTCCTGGCCCACAAGGAAATACGACAGGAGTGTCCCCTGCCCCTTTCTCTGCCCGAACTACAGTCACGAACCTGGTGCTGCTT
CTGACCCCATCGTTCCATGGAGAACGGAGAATGCACTCCAGAGAGTTTTGGACCCAGAGGGACTTCATCGCTTTCTGCGAGGTGGAGGGGTCCG
CCTGGGGAGTCCAGGAGAGCTCCTCTCTTCCCCTGTCCAGTCAGTAACTTTCAACTGTTGGTCTGACACCTGTGTTAATCTCTGACCTGAAAGT
GAAGATACACGCATTTTTACAACAGCCAGCCAAACAGAGAAGACTCAGGTGACTGCGGGCGGACTGGGCCACCTGCGAGGAGACAAGAGAGGGT
GGGTGCAGAGGAAGGGTTTGAAGGGTGCACATTTCACCAGGCGAGGTCACTTTCAACCGGTGTGTACACACACGGGTGTCTCTTTTTTATTTCT
TCGGGAGGGGGGAGGCTATTTATTGTAGAGAGTGGTGTCTGGATGTATTTCTTCTGTTTTGCATCACTTTCTGGAAATAAACATGGACCTGGTA
AAAAAAAAAAAAAAAAAAAAAAAAAAAAAAAAAAAAAA
MOUSE SEQUENCE - CODING
ATGGGCATCGTGGAGCCGGGCTGCGGAGACATGCTGACCGGCACCGAGCCGATGCCGAGTGACGAGGGCCGGGGGCCCGGAGCGGACCAACAGC
ATCGTTTCTTCTATCCCGAGCCGGGCGCACAGGACCCGACCGATCGCCGCGCAGGTAGCAGCCTGGGGACGCCCTACTCTGGGGGCGCCCTGGT
GCCTGCCGCGCCGGGTCGCTTCCTTGGATCCTTCGCCTACCCGCCCCGGGCTCAGGTGGCTGGCTTTCCCGGGCCTGGCGAGTTCTTCCCGCCG
CCCGCGGGTGCGGAGGGCTACCCGCCCGTGGATGGCTACCCTGCCCCTGACCCGCGCGCGGGGCTCTACCCAGGGCCGCGCGAGGACTACGCAT
TGCCCGCGGGGTTGGAGGTGTCTGGGAAGCTGAGAGTCGCGCTCAGCAACCACCTGTTGTGGTCCAAGTTCAACCAGCACCAGACAGAGATGAT
CATCACTAAGCAAGGACGGCGAATGTTCCCATTCCTGTCCTTCACCGTGGCTGGGCTGGAGCCCACAAGCCATTACAGGATGTTTGTGGATGTG
GTCTTGGTGGACCAGCACCACTGGCGGTACCAGAGCGGCAAGTGGGTGCAGTGTGGAAAGGCAGAAGGCAGCATGCCAGGGAACCGCTTATATG
TCCACCCAGACTCCCCCAACACCGGAGCCCACTGGATGCGCCAGGAAGTTTCATTTGGGAAGCTAAAGCTCACCAACAACAAGGGGGCTTCCAA
CAATGTGACCCAGATGATCGTCCTGCAGTCTCTCCACAAGTACCAGCCCCGGCTGCACATCGTGGAGGTGAATGATGGAGAGCCAGAGGCTGCC
TGCAGTGCTTCTAACACACACGTCTTTACTTTCCAAGAGACCCAGTTCATTGCAGTGACTGCCTACCAGAACGCAGAGATCACTCAGCTGAAAA
TCGACAACAACCCCTTTGCCAAAGGATTCCGGGAGAACTTTGAGTCCATGTACGCATCTGTTGATACGAGTGTCCCCTCGCCACCTGGACCCAA
CTGTCAACTGCTTGGGGGAGACCCCTTCTCACCTCTTCTATCCAACCAGTATCCTGTTCCCAGCCGTTTCTACCCCGACCTTCCAGGCCAGCCC
AAGGATATGATCTCACAGCCTTACTGGCTGGGGACACCTCGGGAACACAGTTATGAAGCGGAGTTCCGAGCTGTGAGCATGAAGCCCACACTCC
TACCCTCTGCCCCGGGGCCCACTGTGCCCTACTACCGGGGCCAAGACGTCCTGGCGCCTGGAGCTGGTTGGCCCGTGGCCCCTCAATACCCGCC
CAAGATGAGCCCAGCTCGCTGGTTCCGCCCCATGCGAACTCTGCCCATGGACCCGGGCCTGGCATCCTCAGAGGAACAGGGCTCCTCCCCCTCG
CTGTGGCCTGAGGTCACCTCCCTCCAGCCGGAGTCCAGCGACTCAGGACTAGGCGAAGGAGACACTAAGAGGAGGAGGATATCCCCCTATCCTT
CCAGTGGCGACAGCTCCTCTCCCGCTGGGGCCCCTTCTCCTTTTGATAAGGAAACCGAAGGCCAGTTTTATAATTATTTTCCCAACTGA
HUMAN SEQUENCE - GENOMIC
ACCATGTTGGCCAGGATGGTCTCAATCTCCTGACCCTGTGATCCTCCCGCCTCGGCCTCCCAAAGTGCTGGGATTACAGACATGAGCCACTGTG
CCTGGCTGATACTTAACTTTTTTATATCTCAGTTTCCTTGTCTGTAAAATGGGAACAATGGTTCCCACCCAGTTGCCTTCACACCATTGTGAAG
TCTAATGAAATCAAAGGTATATTGACTAGTCTTAACCAAAAAAAAAAAAAAAAAAAAGAAGAGAGAGAGAGAGAAAGAAAGAAAGGAAATAAAG
GGTACGAGTTGCCTGGGGATGTTCTTTTTTTTTTTTTTTTTGACAGTCTCTCTCTGTTGCCCAGGCTGGAGTGCAGTGGTACAGCCTCAGCTCA
CTGCAACCTCCGCCTCCCGGGTCCAAGCAATTCTCCTGCCTCAGCCTCCCTAGTAGGTGGGATTACAGGCACCTACCACCACGACCAGATAGGT
TTTTTTGTATTTTTAGTAGAGATGGGGTTTCACCATGTTGGTCAGGCTGGTTTCGAACTCCTGATCTCAAGTGATCTGCCCACTTCGGCCTCCC
AAAGTGCTAGGATTACAGGCATGAGCCACTATGCCTGGCCGGGATATTCTCTTATCTCATCCTTTACCCCTCAACCCAGAGTGGTGATAGGAGG
AGGGGGTTAATTAGGAAGAACTGGGGTTTTTACAGTTTCCAAACTCACTGGGAGTCAAGGATTGAGACAAAGTACAAGCTAGATTTGGCAACAT
CCAGGGTCATGCATGGGAACCTGGGAGCCCCATGCCCCACTGCCTATTTGCAGGGCCCTGGGTCTTGGGTGGCCCTAGTTCAGTGTGTGCATGT
GTGTGAAATGCAAGGTGATGAGGTCAGTATCTCAACTCCCAGTTACTGGTCCAGTTGCTAAGAACTTGCCTGACCATAACCATCCTTTCCCAAC
TCACAGAATGTCAGGTTCTGATGCCAGTGATTGGAAAGAAATGGCGGTGAGCAAGGAGAGGGGGCAATGCTGGGCAGCCATGCCACCTTTATGT
CCCCTGTGGCCAGAGACGTGGGTCCAGGGCCCTTCTCAGTGCCCAGCTCGGTCCAGCCAGCTGCCCATGGCCCTGGTGGGAGGAGGTAGGCTGG
ACGCCTTCTGACACTTCCTGGGTAACTGAGCGGGGCAAGCAGCAAACAGCAGGCTCCTGGGGAAATCTCAGACTTGTGTGTCACAGCGGATCTG
TGTCTGGGGTGGTAAGTGGGGTTAAACTCAAGGTCAGACCTAGCCAATCCATTTTCGCGCCAACATGGCCTTGGGTGTCCTCCATTCCAGTGGC
TGGGACCCAGGTGCCCACTCAGCTGGAGACAGCACTGCTGGGAGCCTAATGCCTGGTTTGTCCCTCTGCTTCACACACTGGTTGCTTGCCTGAT
TTCTGCCTGTGCCTAGGATTTATAAGGAAGGGAGTTGTGGCATGTGGATCCCAGGCCTCCGTTCTACTCCCAAATTTCAGGTGCTAATCTCTCC
ATGAGTTTCAGTGACCTCAGAAAAGGAAATGGGATTAAGCTGCAATTTAAGGGGTTGTGGGAAGACAGCTGAAAGAATGTCCCAGTCTTCAGTA
GAGGAGGTGGGAGGTGGCTGTAAACTGCCTAAATCGGGGGAACACTACCAGGAATCTCAAATTGCTGTACAATGCAGACACAGTCAATCAGCCT
CCACCTACTTAGATCTCTAAGTGCTTAGAGATCACCAACCTGTCCCAGGCTCCTGAGTCTATTACCTCACTCCTCCTTGTTCTCTGCCTTTCCC
AGGCTACTGTGGTTATGATTTGTGGCAGAAGTTGGGAAGGCAGAAAAGGAGAGAAGGAGGGAAAAGGACAATCTGAGAACAAGCTGGAGGGCCA
AAGTGGACCCCTTGCAGAGTTGGCAAGATGGCAAAGGGGATGCAGTGGCAGGTGAATTAGTTCAGATGGGCAAGAAATGTAGGATAAGTCAGCA
AGCTGCCTTTGCGCAGGAGATCCCTAGGTGAGAAGGTAAGAAGGGCTTCCACATGCGGCAGTAGCCTCAGTGTTTCTGGAATGGAGTTTGTATA
TATGTTGTGGGTGGGGTAGGGGGAACAGAAGAAGAGCTGCAGCATCCTGCCTTTGAGGAGGAAGATGGTACAACAGGAAGGCCAGGAGGTGGGC
TGCAGGTAAAATGTCCCCGTGAGACAGGGTTGAGGGAAGCAGAGGGCCAGCTGCTGGGCCTGCAGCCTTCTTGCCCCCACTCCCAGCTCACCCG
CCCACTGCGAACACTCCTCAGCTCCCCCACCCCATTCTGGCCCACAGCCCTGGGGAAGCGACATGGCGTTCTCACCACGGACCCAGTGCCGGCT
GTGGCTTCTTCCCTCTGACCTATTTTTTAATCATTTGATGGGATTTTTCCCCCCCAAACCAAACCCCCAGCCCCCTGGGCCCGTCAGCTCCACC
GCGTGTCAAAACCACCCCTGTCACTTTGCGTTGGTTTCTCAGTCCAGCCAGCCCCTCCCCGCAGGGCCTGAGAGCGATGGCTAATTACCCAGCT
GCTGAACGCCCCCCACTCCTGCCCCTCCACCCCTAGCCCTGACAGAGGGGCCCTGGAGGACTGCTGGGAGAAGCTGGGGCCTCCTCACCCTCCC
ACTGCTTGGCAGGAAGTCCTTCCTGCTGTCTAACCTCCATTCTTCCCTCTGTCCTGCCCCTAGTGGTGCTGAATCGCAGACACAGAGGCTGTTG
GTACTATAAGGGTCTTTGGGATACCTCCTTAAGGGACAGAAAGGGGCAACTAGTCTTCTGTCCTGGGTTTAGGGATGTCTTCTGTCACTCTGGC
TCTCTAACAGAATTCTAGGGGATGGTCCTTGCTGTCTCATTTTACAGAGCAGAAAACTGCGACACACAGAGGGAAAGGGCCTAGCCCAAGGTGA
TCTACGCCAGTGGCGGAGCCAGAATCAGAACCTAGAGTTCCATGTTTGTCTTTCCACCTGTTTCTGGGTCCTGTTTCTGGTATGTAGAGGGCCC
ACCCCCACATACCAACTAGGTCTGTTTCTGTTTCTGTGCAACTCCGCTGCCAGCCCTGAGCAGGTGTGTTCCCTGCCATGCCCTCTCTCCCTTG
CCCCCTGGCCACACAGAGCTTCACCCTGGCATTTCAGGGCTGGGCAGCTTGCTTCAGGCACTATTTAGGAATGGGACTCTTTCCCCCAATACCA
CCCATGCCCTTTTCCCTCTTCCTCTCTCATTCATCCCTAACCAAGTCCGTAAGTCTGCTCTGTCTCACCCGCACCCTGAGACATTAACCAGGTG
GACAAGGCAAAAAGCTCTAGGCCAGACACAGGAAGGCCAGAGCCAACTCACAGATGAGGCCTAGTCCCAGATGGACAGATAGACATGCAGGAGA
TACATGGAGCGCTGGATAGCCCTGGAGACACGGACAGTGACATGAAGATGCAGAGACAGACACAAACACGCTACAGAGACAAGGCAAGTTCCGA
GGCCCACAGGTAACAGACAGACACAGAGACGCAGACAGAGGCACAGAACCTACACTCAGGACCAGCGTGCAGACCTCTCACAGGTCCCAGGGAT
AGGAGTGGACGTAAAGAGGATACACAAGACGCAGGCAAAAACACAGGGACCCAGAGAGGGGCCGAATCAGACCAGAGGAACCCAGACACTCAGG
AGCCCTGAGAGACATGGGCACACAGGGCTGCCCACAGATCCCAGGAAGCCAGAGAGAACCTTGGGGAGAGAGGAGGCCCCAGGGGGTGGGTGGC
CCTCTGAAGGACAGCGAGGCCTGGCCTGGCAGAGCCTGACTGTGCCAGGAAGTCATGCCAAGATGGCAAGATGGGAAGCAGAGAGGAGCTTGGC
AGCGTGGGGGTTAACCCCTCAGCCTGTGCCCTCCCTCCAAGTCCCTTTTCCTTGGCTCTTCTTTTTCTTCCCTCCTCTCCCAGCTCCTTTTACC
CAAAGCCCTGGATATTTCCCGCCTCCCCAGCCCAACTCCTGGTAACCAGACCCTGCTTCCTGCCTGAGATTAAGGGCCTGTCTGCTGAGAGGCA
GGAAGGGTGGGGGCACAGCCTCTCCCCAGTGCCTCCTGAAGCTTGGGCTTGCCCATCCATACTGTGGCATGCCCTCCTGATAACAGAGTTACCC
AAGTACCTGATGTCTGAATATCCCTTCAGGAGGGCGCAGGAGCCCTAGCCTGGGAGTGCAGCGGGGGCGGCCTACTGCATGCGTGGGTGTGCAT
GGAGGGTGTGGGTCTTATGTGAAATGCTGGCAGGGGTGAGAGCAGCTAGAGATAGAGAGAGGATAGATGATTCAAAATAAGATGCCACGGAGAA
GGAGGACTGAAAGGCACCCAGGCCTGGCCTGGAGCCCAGGGGTAAGGGCCGCTCAGGCTTGGTGGGGGGTGTGTGGGGAAGGGTGACCCAGCCT
GCAGATGAGAGCCTGGAGCCCTTGACCTTGGGAGGAGCCCTCTGTTCTGTGTTCACACTGACCACCTGGTGCTGGGTGCTGGGGACTCGGAACT
CCTGCAGCTGTCACGGGGCGGGGGGGTTCTACAGGTGCTTGTCCCTCCCCTTCCCCCCTCTCTGGCTGTTAATCTGATGGTTCCGGCGATGCCC
TCTGAAGGACTGGCAGTCTGGGCCCAGAGGGGGAGGGGATTCAGGGCACGCGGGGTGGGCAGAGTCAGATGCTGAAGGAGCTCTGGGATGCTGG
ATGTGGGGCAAGAGCAGGCGGTGGGGCTGCAGGGACTGGATCGCAGAGATTTCCCTAGCATAGCTCTGCATGGGGTGTGTGTGCACATGCGTGG
ATGATGGCGTGTGTGCACGTGGTGTGTTGTGAGTCTACACCCCAGGAGCAAGAGGGCTGAGGGGGTCACTTTGCTAGGAGCTCTTTGGGGGCAG
GGACTGACCCTAATTTATCTTTGTGATCCCAGATCTCCAAGGGCCTGGCTTATCCCAGCGCTCAGGTACCCAGGGAATGGTATATGGGTATCAG
GAGGGGAAGCCTGTCCCTTTCTAATATGTGAGTCTTATGAACAGAGGGGCTCTCTTTTCGCCCCAGGCCCTGAGTGGAGGCTGAGGATGGACCT
GAAGGACACTTTGTGGATGAGCCACTAAGCCAGAGGGAGGAAAGGGCAGGTGTAAGTGGCTGGGACTACAGGAAAGAGGGCTTAGCACAGTTTA
TGCCTCTGTTTTGTTTCAGCCTTAGGACAGCATCCCTCAACCTTCACATGACAGACCCAGTCAGTCCTGGCAGAGGTCTGATAGCTCCATTTAC
TGACAAAAAAAACCCAGAGAGGTTAAGATACTTTAATAGGTAGCACAGCCAGTAAGGGTTGGGATTGAGGTTCCAACCCAGCCACCCAACTAAG
TCTCCCCACCCCATTTCTCTCTGCCCCAGCTCCAGCACCCCCAGGCCTCTCTGCCTCTGCAGACTTTCCTCCCATTGGCCCATCTCTGTCTGTC
TCCTTTCTTTCATGCCCCTCTCGCAGGCCCAGCCTGAGACTTTCCCACTGAGTCACGATCTGGGTCCCTGACTTTGTGGTCCCTGTCCCTCTCT
GGGTCTAAATCTTTCTTTCAGAAATGCACCCTAAGCCTCTCACTTTGCTCTCTCTTTCTTTTCACGCCTTCTCCCCAACTCGCTGCCCCCTCTC
TTTTACTATCTACCCTCTCACAGCCTCTAAGGCTCTGCCCAGCAGGGTGGAGGGTGGAAATCCTGGGTCCTGGTGCCATGCCATGCTCCACTAA
CAGTTTCCAGTAGGCTGGGCGTGGTGGCTGATGCCTGTAATCCCAACACTTTGGGAGGCTGAGGCGGGCAGATCACGAGGTCAGGAGATCGAGA
CCAGCCTGGCCAACATGGTGAAACCCCATCTCTACTAAAAATACAAAAAATTAGCCGGTCGTGGTGGCATGTCCCTCTAGTCCCACCCACCACG
GGAGGCTGAGGCAGGAGAATTGCTTGAACCCGGGAGGTGGAGGTTGCAGTGAGCTGACATCGCACCACTGCACTCCAGCCTGGGTGACAGAGCA
AGTGACTCTGTCTCAAAAAACAAAACAAAACAAAACAAAACAAAAAAACAGTTTCCAGTAATAGCCGCTCCTCCCCAGCTGCCCCACCAAACCC
ATCCAAGAGCCTTCCAGGTGCCTAGGGAGCTCAGCACTCCATACGTTTTCAGGAGAGCACCCAGGTGGCTGGTAGACTCTGGGGTGAGGAATCA
TCTGGTCCTAGAGCATCAATAAGGAAATCTCAATGCCTGCAGCCACCCTCCTCAGGCATGAAACCTCCTCCACCGCCCTCAGTGTGCATACCCA
GCAAGCTCAAGGCACACTGACTCGCAGGATGGGACTTGGAGATGGGACAGCAAAATGGGAGGGAGCCTGAGAGTGGGGTGCATGGACTCCCTGA
AGTCCAACGCATCACCTGGAGTCCTTAAACATACAGATTCTAGAGACCCGCCACAGGCCTGGGCCTCAGAATCTCTAGAAGCAAGTCCCAGAAA
TCCACATCGTTGTGGCTTCCTGGCATAGTCTGCAGCCCACAGGTGAAGCAAGTGCTCTGTGACCTTCGGGGAGCCTCTCTCATTTTGAGTCTTC
ATGGGAAACAGTTTGAAAGGAAGCCCTGAACGTTTGCAGAGCACAACAGCCTGGAGAGAACCAGTGTCTAGGCTGTGGGATTAGGCATGATTTT
CTTTTCTGTTTTCTAAACCTTTTCTTTTCTTTTTCTTTTTGTTTCTTTGAGACCGAGTCTCCCTCTGTCACTCAGGCTGGAGTGCAGTGGCACA
GTCTCAGCTCACTGCAACCTCCTCCTCCTCGGGTCAAGTGATTCTCTTGCCTCAGCCTCCTGAGTAGCTGGGATTACAGGGGCCAGCCACCACA
CCTGCTAATTTTTATTTTTGTTTTTGTTTTTGTTTTGAGATGGAGTCTTGCTCTCTCTCCCGGGCTGGAGTGCAGCGGCACGATCTCGGCTCAC
TGCAACCTCTGCCTCCTGGGTTCAAGTGATTCTCCTGCCTCAGCCTCCTGAGTAGCTGGGATTACAGGCGCCCATCATGCCCAGCTAATTTTTG
TGTTTTTAGTAGAGATGGGGTTTTGTCATGTTGGCCAGGGTAGTCTAGAACTTCTGGCCTCAAGTGATTTGCTGGCCTTGGCATCCCAAAGTGT
TGGGATTACAGGCATGAGCCACCACGCCCGGCCTGTTTTCTAAACCTTTTCCAGTGTTAGTATGATTCTTCCTCATTCTTGTGGCAAGAGCACT
GGCCTGGGAGTCAAGAGACCTGGGTTCTAGCTTCAGCTCTGCCACAAATGCACTGTGTGAGTTTGGTGTCTTATTCCATTTTGCATAGTTACCA
TCCACCGTGTCTCATAAAATGTGATGCTGGTTCGAGGCCATGTCTCTCTCATTCCTGGACACTTATCTTGTGTTTCTTCACTCCGGTCTCACCA
GGACCACCCTCATAATGCTCCCTGTATTGATGGGATAGAGAGGGCTGGGAGGAGCAGCCATGGGGAAGTGAGGGGATCCCAGGGGGCCTCATCT
CCTCACTCCTCTTCTCCCACCTTTCCAGCCCCAGGGCCCGGGAGGAGGGTGAGGTGGCAGCTGGAGGAGAAGGTGTCACTGCCAGGGCCCTTAT
TCTCACCCAAGCTGGCAGAGGGGCGGGACTGGCAGCAGTGACACCAGGGCCACTCAGCCCCCGCTTTTCACAAGCACTTTCTTTTTTGGGTGGA
AGAAATTGGAGAGAGGGGGTGAGAAATCGGGGATTGAGGAACCAGGCTGTAAGACCCTATAACAGCCTGTCAGGTTCTAGAGAGACCACCCCCA
CCCTTCCTCTTCCTCCAGAGGCATGGCCCCAGGGCCACCTGATTCTGAGATTCATAATTCCACCTGCCCCAGAAAGCTGGGTGGGAGCTCATGT
CATCCTGTTCAGAGGCAAGGGGATGAATCACTTGACCTGAGGATTAATGTGGAAAAGAGGAGGGGGAGAGAGAGGGAGGAGCAAGACTCCATTT
GATCTTCAACAGCCAAGCACTGAGCAAAAATGTCCCCCAAAGGGGTGTCAGCATTCAACAGTCTCACATCTCCTAGGGAATCAGTCATTCAGGA
GGGTCTTACTGAAAGCTCTCATGTTTCCCCAAAAGTAGTGAAGGATCCCAAGGTCACCCTCTCTTCCAAGCAATGCTCCCCCACTTTGAACATC
AGGCAGAAACTTCCCTGTTCCTTAAGGTACGGAGAAATGGTGGGTAAGGTGTTGGGGAGGAGGCCAGTCCTGTGTTTCTGGTACTGTCATGTAT
CCGGTCTCCAACCTCAAAAGATGTGCTGATTCTCTCTCCCCCAAGCCTCCCTGGGTCATGCCCACCTCCTGGAGTCCCCAGCTTTGCCTTTGCC
TGCCAACTTGCCTCTTGGTTCAGCTCTATTTGGGAGCAGAGAGTCCCCATAGGCCCCTAAGGGTGAAGCCCTGTCAGGCTGGGACAGAAATGTA
GAGCTGGGGCCAGTTCCCAGGGGAGAACTGGGGAGGACTGGGGTGAAGGTAGAGAGAGGAGAAGTTAAACTTTAACATGACAAAAATATTAAAG
TATTTTTCAAAGTACTCTTAAAAATGTAAATCTTTATCTATATACTGATATGCAAAATATTCCATGACACCTTGTGGAGTGAAAAAAAAGTACA
CATTATAAAGCAGCATGTGTAGTGTTTATATAAATTATATATTTGTGCATGTGTGCAAAAGACAGAGACAGAGAGTATATAACGAGTGAGGTCT
CAGAGGGTGTTCACTGAAATGTTAATCCTGTTTGGCTTTAGGGGGTGGAATTTGGGGTGATTTATACAGTCTCGTTTTTACTTTTCTGTGCTGC
TTAAATTTCCTATATAAGTATGTTTCATCTTTATACACAGGAAAACTCCCAATGATGCTATTTTCAATTGGGGGGGGGGAAACGGATACTTTTC
ATCATAAAACGAGTCTAATACATTAGTTAACATAAAAGGAAAATGGAGAAAGAAAAGCCTCCATCGTCCTATCTCCGAGCCACCCCTTCAGAGC
ATGTGGTGGGTTTCCTTCCTGTCAGCCGGCTCTGAATCTGTTCAAAGCAGCTTTTCCATCCAGGTATAATAATAGATTGGAAAGTGCTGCACAC
GTACACCCTCTCATGTAAGGCTTGCAGCCCCCCTGAAAAGTAGATCTGTTTATTTCCCCCAGTCTATGGATGAGGCACGTGAGGTTGACTTTCA
GGCAAGGAAAATGACTTGCCTGGGTCACATATACCTGGCAGGCAGAGCCAGACCTGGGACTCTCAGCACTGGCAGCCCAATGCTACTTCCAGTG
CCCATGACTGCCATGCAGGGTAGGGGAGCTTTTGTGGGCCCTGCCAGGCCCAGCTCAGGGCCCTGCAAACCCCTGGCTGCTGCTGATGCAGTGC
GCTTTAAGGAACATTTCCTCTTCTCCATCAGGTTCCAGGTCTGCCCCCAGGGCTAGAGCTACAAGATGCAGCCAACTCACCACATCTGCACCTC
TGGTGGGGAGAAAGAGGGCAACCCGAAAGGTCACTTAGCACAGAGTCTGGGCACACAGTAGGTATCAATAAAGATCGATTGAATGTTCATGGTC
AAAGTTCCTTCTAGTCTGCCCGTGCTCCGAGCCTCTGAGTGCCAGGAGAATGCCCAGCGAGTCCCACTTGGGCCATCTCGGAAGGCTTCCTGTA
GGAGAGGGCCTTTGAGCTGAGACTTCAAAGCTGGGCTGAATTTCCCCGAGGGTCCACAAGAGAGCCACGGCCTGGTGTGTCACGCAGCGGAGCT
GACACTCCCAGAAAGCAAGATCTTCGAACTACAGGGTGCGCGCAGCCTCTCGCTTCTCTCCACCATGGGGGGCCCTGCAGTACTCGCCAAGAGC
GTAGAATTTGCCTAGTATTAGCCACGAGAGGGCGGGGTGGGGCGAGGCGGAGCAGCCCCGAGCTGGCGGAGTGCCGGCGACCCGGAGAGCTTCA
TAAAGCCACAGCAAAGCGCTGCGACTCTAGTCACAGCGGCCCGCTGGAGAGGAAGCCCGAGAGCTGCCGCGCGCCTGCCGGACGAGGGCGTAGA
AGCCAGGCGTCACAGCCCGGGCTCCGGTGGGGTCCCCCACCCGGCCCTCGGGTCCCCCGCCCCCTGCTCCCTGCCCATCCCAGCCCACGCGACC
CTCTCCCGCGCGGAGGGGCGGGTCCTCGACGGCTACGGGAAGGTGCCAGCCCGCCCCGGATGGGCATCGTGGAGCCGGGTTGCGGAGACATGCT
GACGGGCACCGAGCCGATGCCGGGGAGCGACGAGGGCCGGGCGCCTGGCGCCGACCCGCAGCACCGCTACTTCTACCCGGAGCCGGGCGCGCAG
GACGCGGACGAGCGTCGCCCCGCCGCCACCCTGGGGTCTCCCTACCCGGGGGGCGCCTTGGTGCCCGCCCCGCCGAGCCGCTTCCTTGGAGCCT
ACGCCTACCCGCCGCGACCCCAGGCGGCCGGCTTCCCCGGCGCGGGCGAGTCCTTCCCGCCCCCCCCGGACGCCGAGGGCTACCAGCCGGGCGA
GGGCTACGCCGCCCCGGACCCGCGCGCCGGGCTCTACCCGGGGCCGCGTGAGGACTACGCGCTACCCGCGGGACTGGAGGTGTCGGGGAAACTG
AGGGTCGCGCTCAACAACCACCTGTTGTGGTCCAAGTTTAATCAGCACCAGACAGAGATGATCATCACCAAGCAGGCACCGTCAGTGCGGCGCG
CCGGCCCTTGGGGCCTCTGTGCCCGCGCCGGAACAAGAACGTCTCGTCTGTTTTTCTGGCTCCACAATGCTTCTGACTCCGTGTCCCTCACTGC
TTTGGCTTCAGCGTAGGGAGACAGGGGAATGGGGTTGTTAGGAGGACAGGGAAAGCTCCGGAGGGGCGTCTGTGCCCAGCCTGTTGCACCAACA
GCCAGAGGACTCACAAGGGAGACGGGTGAGTGCGGGACAGTGAGAAGTCACCTTGATTTAGGGGAAGGGTGACTGTGGCTTCACCTAGAATTGG
TGTGCGCCCCTGCCCCACTCTCTACTGTAGAGGAGTCGCAGCGGGCAGTGAAAGCCTGTGCTCTGGGCGGACAGGACGCCTGGGCCTCCTGTGT
GGGAAACTGGAGGGGAAGGGAGCCCCTTATCTCCGGGCCCCCTCCGCCCACCTCCCCCGGCTCCTTTGCTGCTGGTGTGCTCAGGTCAGCTTTA
GTGGTGGTAGTGGTGGTGGTAGCGGTGGTGGTGGTGTGTGTGTACGGGGGGAGATTGGGATTTGGTGACATCGAGAAGCAGTCGCCAAGTTTCC
TTTCCGGTCTTACTTTGACATCATATGTCTGGTGTGTGTCTGTGTGTGTGTGTGTGTGTGTGTGTGTGTCTGTGTGTGTGTATGTGTGTGTGGG
TGTGTGTGTATGGGGGCTAGTGCACTAAAGCTTGTAGGGGGCACAGATCCCTTCCAGGCACAAATGCCCACGGGCTGGGCAGATCAAGCTCACC
CAGGGGTCCAGCCTGGTAGCCAGCCCCACACTGCACCCTTTGAGGCTGGTCCAGTGAAAACCTTCCCCACACTCCTGTCCAGAAATTCACCGGT
TCAGCCTGCAGAAGTGGGGAAGGGGTGTCCCATGGCTTCATGGCTCAGGGTTCCTGAGCCCCGTGCGTGATGGGGAGAGTTTGGGGCTGAGGGT
GCTGCTTCCGGATAGAGCCTCCTGCGCAAGGAAAGAAACAGAAACCTGTGACTTGTGTGGTATTTGTTTAGTAAGCAACCCCCGGAGTGGACTG
TGTCTGATGTGGGCTGTGCGCACCCACCCTTCCCAGTGCGGCCCATGTGAGCAGGGGACCAGCGAGGACCAGTGTGGAAGGGCTGTTGTCATTG
GTGGACCTGGGATGCTGGGTCCCAGGTCCGAGAGGTGTGGATACCAAACGTGGGGCTTGGGGTGGAGGGGAGAGGGAGAAGGCCATGTTGGACC
CCAGAGGTTGGTATTCGATCTGGGCATTGCTGGAACATTCTTCCCTCCAGATGATTTTTGTGGGGTAGCCTGGGACTGGGGAACCTGTTGCCAG
CCACAACTGGCTTCAAGTTACTGAGCTGTTCCACTTTCCCTGGGATGAACCCAGGAAAGTTGGCGTGTTCTTTGATGGCTCAGCCTTCATCTCT
TAAGCCTTCTGATAATTTCTCTCTGCTCCCCCCAACCCTAGTCACCCTTTGGGTGTACCCAGACACAGCCCAAGCTTCTGGTCCCAATGTGTCT
GAATTGGAAGGGACCCAGAAGATCCTGTCCCTCTCACTGCACAGACAGGGAAACAGGCCCAGAGATAGCAAGAGACTCTTCCAAGGTCACACAA
CACACTCATTGCAGGCAGAGCTGGCACTACAGCCCAGATAGTATTTGCTTGGGTTTCTTTCTCTGGCCCCCTACGGCCACCAGTAAAAACAGCC
ACCCACGCTCTCTGAAGGCCCTGATGCATTCAGAGTCCTTTCACTTCCATTATTTCACTGTGCGTTAGGTAGGCCAAGCAGTCTCCCCATTTTA
CAGTTGAGGTCACTGAGTTGCAGGGTCAGCTAGGGGCTTGCACTCCATCACACAGCAGACCAGAAGTGGGGCCTCTTAACCTTCCACTCTGTTG
TGTGGTCAGGAGGACTTCCCACCCTGGGCCCTTGCTGGGGTCTCCCCACAGCCTCTGCCAGGCCTCTGCCTCCTCCTGCCAGTCTTGAGGGATC
GGGTGGATGGAGCATCAGCCAGCACTCTAGGGAGTTGGCCAGTGCTAGGGGGCTGCCTCTCTGCCTGTAGAGCCAGCTTCAGGGAGGCTGTAGA
GCACTTCAGGGAGCCACTTGAGGGGATGATTCTCGAAGTGTGTATCACCATCTCTACTCCCATGGCCTCCTTTCCTTGGTGTTCCTGCTGAGTA
ATTCTCACTTGACAAGTTTTTTTGTCCCCCTTTTAGATACATACACTTACATTTTTTTATTATGAAAAATTCCACTACATACAAACATGGACAG
AAGAATGTAACAGACTTCCATCCCCAGCTTCCACAGTTGTCAATATGGGTCCAATCTTGTTTCTGCATCCTCACTTCGCTTCTTCTGTCTGGAA
CTATATTAGTAATATAGTAATTACTAAGTACATTCTAGATATTATTTTATCTCTGCACCTCTAAAAATGACTTTTTAAAATGAATGCATTACCA
TTAGCAAACCTGAAAAGAATTTCCTAAATATCACCAAGCTTCTCTGATTGCTTCATAAATGTGTGTGTGTGTGTGTTTTCCAATCCTCACACTT
TTTGCTCCATTCTCAGGGGCTCCATACCCGGGGTCCCTGTTGAAGGAGGCAGTGGCTCTTCACTGTTTCTTTTGGTGCTTTTTCTTGGATCAGG
CTTCATAGACCCCACGCTGCCATGGAGGGGACAGAGGACACACAACCCCAATTCAGTCCCACTAGCCCCAGGTTACCTCCATACCTTCTCAGCT
TGGGCTCCTGAGATCTGGGAAATCAGTGGCCATTTCCTCTCAAAACAGGCTTTTGGGCTTATAGCACAGCTTCCTTTCCGTCTTCCTGCTTTTG
TGGGCTGGCCTATCCCTCCGTGGGTTCCAGTGACCCACCAGTCACACCACATCACCCATGACCTTCTTCCTTTTTGAAAGGTTCTCCCACCTCA
GGCCCACCTACAGCCTACAGCCCCACGGCTTTTTGCCTTGAGCTGACTCACGCCGTCCCCGATTCTTCCTCTCCCACCCACCCTGGATCTATCC
CTCGGTATTCACCAGCGCCAGTGGCATGCTCTTCCCAACGGGCAGAAATGGGGCACCGCACACGGGGTGATTCCTGGGTCTCCAGATTGATCAA
GGCAGCTCAGATGGGGCTGAGACAGGGGAACTGAGAACAAAACCGTCGATTATCCCAGTAGGAGACTTTGGTGTCTACGTTTGTGTCTGGAGAT
TTGGGGCAGCCTGATAATGCCCCACACAGGCTTGTGCGAGGGTTTCTGTGTGGCAGTGTGTGTGTGTTGCGAATGTCTTGGCGAGTATATAATA
GTTGATGGTAGACATTCTCAGTAGAGGAAGAGAGAGGCCTCTGTCTCTGTGGGTGTGTGGGTGTGTATTAATTGCTGGCAATCCCCACTTTCTG
GGTGTGATTGGTTGATTATTATAATTAAGATAACTTTGGGTAGCTGTGTTGTGTTAAGCTCAATCTGCATTTGGATTTGTGCATGTCAAGACAG
AGGTTGGTAAAACAACAATTTCAGTCCCACACTATCCTTGCTATCCTCACAGTCATTACAATTTTTTTTTGTCTCCACTTTCCCAACTCCAAGA
ACTTTAATTTTTTTATTTCTAATATTTTTCATGGGCCAGGAAGTGGACTTGAACCTCCCACATAGATAACTTTAGTCGATAACTTTCTACATCT
TTTTTTTTTTTTGAGATGGAGTCTCACTCTGTCCCCCAGGCTGGAGTGCAGTGGCTCAATCTTGGCTCACTGCAACCTGCACCTCCCAGGTTCA
AGTGATTCTCCTGCCTCAGCCTCCCAAGTAGCTGGGATTACAGGCAGAAGCCACCAAGCCCAGCTAATTTATGTATTTTTAGTAGAGACGGGGT
TTCGCCATGTTGGCCAGGCTGGTCCCAAACACCTGACTTCAAGTGATCCCCCCCCTCGGCCTCTCAAAGTGCTGGGATTACAGGTCGGAGCCAC
CGCACCTGGCCCAACTTTTCAGATCTTAAAAAATTTTTTTTAATTTATTTTTTATGGAGACAGAATATTACTGTGTTGCCCAGGCGGTCTCAAA
TCCCTGATCTCAAGCAATCTCCCGCCTCAGCCTCCCAAAGTGCTGGGATTACAGCCATCAGCCATTGTGCCCAGCTAAAAACATTTTTTTTAGG
CTTAAGCATGCTTAACTGGTTTCATTTGTCTTGGGGCAACTGAAAACAACTTTAATTAAAAATTTTTTTTAAATGAGACATGGTCTTGCTCTGT
CACCCACCCTGGAGTGCAGTGGTGCGATTATGGCTCACTGCAGCCTCAACCTGCTGGGCTCAAGCAATCCTCCCATCCCAGCCTCCTGAGAAAC
TGGGACTATACGCGCACATCACCATGTCCGGCTAATTGTTTCTATTTTTTGTAGAGATCGGGTCTCACTATCTTGCCCAGGCTGCAGCTTTGCT
TTTTGTCTGGCTGTTTGCCACTGGCTGTCTGAGTCTATTAAGTGGATCTGAGGTTGGTAAGACGAGGAGTTCCCTTGATGCCCAAGCACATTTT
GTACTTTGGTAGGTACGACACATTGCCAGAATACCCTCCCAAAAGGTGCTACCAATTTACGCCCACACCAATAGTCTATGAGAGGACCCATTTT
CTCACAGCCTCATCAGCAGTAGATATTATCACATTTTTTTTTTTTTTGAGACAAAGTCTCGTTCTGTTGCCCAGGCTGAAGTGCAGTGGCATGA
TCTCTGCTCACCGCAACCTCCGCCTCCCAGGTTCAAGCGATTCTCCCACCTCAGCCTCCCAAGTAGCTGGGATTACCGGCATGCACCACCACGC
CTGGCTAATTTTTGTATTTTTAATAGAGATGGGGTTTCACCATGTTACCCAGGCTGGTCTCAAACTCCTGACCTCAGGTGATCCACCTGCCTCG
GCCTCCCAAAGTGCTGGGATTACAGGCGTGAGCCACCACGCCGGGCCAATATTATCAATTTTTAAACATTGACCAATATGACTGAGAAGAACTA
TCTATCTCCTTGCTATTTTAATTTGCACTTAATTACAGTGAAGCAGAGCCTGTGCCAGGAGCTCCGAATCTGGGAGACGTATGACGACCCTGTG
AACCATCCCAAGGCTACTTAGTTCTTAGGGCCAGCAGAGGAATTTGGGGTCTTGGACCCTCTCTTATCAGGGTGGGATGGTAAGTGGCCTTTAG
GGACGCTCAATTTGACACCAGACCCATCACCACTCTCGGTTCCTTCCCAGCAAAGTGTCATGTAAATCAGCGGCTATTTTGGGATTCCAGCTGG
TGGTGATTCTAGACTTTTAGGGAGAAACCGAGATCTCCTTTCTCTTCTCCCTTTTCCCCAGCCTCTTCCTGTGTCTCCATTTCCCTCTACTAGT
GATAAAAACAGGATGAGCTGGGCATGGTGGTGAGCACCTATAACCCCGCTCGGGTGACTCGGGAGGCCAAGGCAGGAGGATTGCCTGTGCCCAG
GCATTTGAGCAGAGCCTGTGCAGCATAGCAGACTCCATCTCTTAAAAAAAAATCAGTATGGCCTGGGCGCGGTGGCTCACCCCTGTAATCCCAG
CACTTTCGGAGGTCGAGGCCAGTGGATCACCTGAGGTCAGGAGTTCGAGACCAGCTTGATTAACATGGTGAAACCCCGTCTCTACTAAAAGTAC
AAAAATTAGCCAGGTGTGGTGGCGGGCGCCTGTAGTCCCAGCTACTCGGGAGGCTGAGGCAGGAGAATAATGGCGTGAACCTTGGAGGCGGAGC
TTGCAGTGAGCCAAGATCACACCACTGCACTACAGCCTGGGCAACAGAGCGAGACTCCGTCTCAAAAAAAAAAAAAAAATTAGCCAGGCGCAGT
GTTGGGTGCCTGTAGTCAAGGAGAATCTCTTGAACCTGGGAGGCAGAGGTTGCAGTGAGCCGAGATCACACCACTGCACTCCAGCCTGGGGGAC
AGAGCGAGACTCTGTCTAAAAAAAAAGAAAGAAAAAAATGAATATGAAGAAATGGGGACCACAGAGCACAGCATGCACTCTGCAGTCAGAAGAC
CTCACCTCTGCTCTCCCACCGAGGACCGTCACCAAACCTCTCGGAGCGTCAGTTTCTTCAAATATAAAATTGGAAATAACATTTTATTTTTTAT
TTTTTCTTTGAGATGGAGTCTCACACTGTTGCCCACGCTGGAGTGCAGTGGCGCCATCTCGGGTCACCGCAACCTTCGCCTCCCAGGTTCAAGC
GATTCTCCTTGCCTCAGCCTCCAAAGTAGCTGGGACTACACGTGCATGCCACCACACCAGGCTAATTTTCTGTATTTTTAGTAGATATGGGGTT
TCACTATGTTGGCCAGGCTGGTCTCGAACTCCTGAGCTCAGGAAATCCACCCTCCTCGGCCTCACAAAGTGCTGGGATTACGAGTCTGAGCCAC
TGTGCTGGGCCATCTTTCGAACTTTTTAACAGCTTTCAGAGGAAAGATGGACAGGAGTTAGACTCTCCATTTACATGTGAAGAAACTGAGATTT
GGGCTGGGTGCAGTGGCCCATGTCTATAATCCCAGCACTTTGGAAACCTGAGGCGGGTAGACCACTTGAGGTCAGGAGTTCGAGACCAGCTTGC
CCAACGTGAAACTCCATCTCTACTAAAAATACAAAAATTAGCCGGGTATGGTGGCACATGCCTGTAGTCCCAGCTCTTGGGAGGCTGAGGCAGG
AGAATCACTTGAACCTGGAAGGTGCAGGTTAGAGTGAGCTGAGATCACGCCACTGCACTCCAGCCTGCATGACAGAGCGAAACTCCATTTCAAA
AAGAAACTGAGATTTGGCTTGAGAATTAACTTATCCAGGGTCATAGCGTAGTAATCTATTTTTCTCAACTCAGGAAACTCAGGGGAAGACAGGC
GTGTGACAGCACCCTTCTAGGACACCTTCCCAAGGTTGTTAGGTAAGTTGTCCTGGGGACCTCTGGGGATTAGAAGTCCCTCAAAAACAGTTCC
TAGGCCCCTGGAACCCTCCCTCCTCCAGGTTTCCTGACTCAGATAGGTCCCTTTTTCCTCATCCTTCAGGGCTTCTCATGAGAGGAGGCGGAAG
TGTGTGGAAATGAAAAATAATTCTGATGAGGATTAGCTTCTGCCATTCTGAAAACTCTGCTCTTCCTGCTTGTGTTAGAAGAGAAAGGCTAAGC
CATTGGAGCTGGTGATGGAATTGGTGCTGGTGGAGGTGGTGGTTGTAGTGACGGTACTACTGGTGGTGGTTGTGACGGTGAAGGTGGTGGCTGT
AGATGGTGGTCCCCGTGCTGGTGCTGGTGGAGATCGTGGTTATCGTGATGGTGGAGGGGGCAGTTGTAGTGACGGTGGTTGTGATGGTGGTGAT
GGGTGGTTACAGTGCTACTGGTGGTGATGGTGCTGGTTGTAGTGGTGGTGGTGACGGGGCTAGTGAAGCTGTTAGTGGTGGAGATGGTGATGGT
AGAAGAGTGCTTCTTTTCTGAATCTGTTACTCTGGCACAACAGCGGAATCATACAGCATCCCACCCCCTAAACTTCCTCTACTTTTCCTGAAAC
AAGGACTTTGAACATGAGCCCCGTGTTCAATAGAGCCTTCTTTCAGGTGGATGGGAAGCTCCCTGTTCAACTAGGATGTGTGCAACTTCTCTAA
AGGCTGAGGGCAGACTATACCGCAGGTGGCAGACCCAGGAGCATGGGAGAGGGAGGGCCTGTGAGTGGGTGTCATGGGTCTGGGAGTGTGTTAG
CACTGTCAGAGCCACGGCAGAGACGGTGGCTGACGAACTGAGGCGTGAGGACCTTCCAGGAACTGCCTCTGGAGAGCTGGTGGTGCCCCCTCCT
AGGAAGCAGCTAAGGTTAGAGACAGGGGTGTGCAGGTAGATGTCGAGCAACTGACCCTCTGAAAGAACATTTTAAAAAACTCTGATTTGTAGTG
TTTGCTGATCCCTGTGGTGTAAATACTCCTGCTATGGATGATTTCAAGCGACTCATGTGAGTCATTGAACACAGAGCCGGGAAGAGACTCACAG
TGCACTGTGATTTAGTACCTCCTCCATGCAGCTGCGATAGACGCAAGTAACCTCGACAGTGCAGACAATAGTAAAACGTAAAATAACTGGGCAG
TGATGATTTTTTAGTTTTTACTCCTTTTTTTTTTTTTTGAGATGGAGACTTGCTCTTGTCATCCAGGCTGGACCGCAGTGGCGCGATCTCAGCT
CACTGTAACCTCTGCCTCCTGGGTTCAAGCCATTCTCCTGCCTCAGCCTCCTCAGTAGCTAGGATTACAGGCGCCCACGACCACACCCAGCTAT
TTTTGTTGTTGTTGTTGTACTTTTTAGTAGAGACGGGCTTTCGCCATGTTGGCCAGGCTGGTCTCGAACTCCTGACCTCAGGTGATCCGCCCGC
CTTGGCCTCTCAAAGTGCTGGGATTACAGGCATGAGCCACTGTGCCGGGCCTACTTTGACTGTTAATATGTCTTGAATTGCAAGTTTATATAAT
TTAATTTTTAATTATGGCTGTGTTTAACCACTGGCTGGCAAACTCCCTAAACACCTTCCAGCTGGTTCTTGTGAGTGGGACGAAGCCGGCTACA
GCACACCACTGATGCCTGGGCACTGTTGCAGGGGGGACTGGCTCTCAAGCTGGAGCTGATGGGTGCTGGGGGCTTTCTCTCTTCTCTCCAGCCG
GATGTTCCCATTCCTGTCATTTACTGTGGCCGGGCTGGAGCCCACCAGCCACTACAGGATGTTTGTGGACGTGGTCTTGGTGCACCAGCACCAC
TGGCGGTACCAGAGCGGCAAGTGGGTGCAGTGTGGAAAGGCCGAGGGCAGCATGCCAGGTGCGCGCGCCCCTGGGAGCGGTGGGCTCTGTTTCG
CTGGGACTGGGCGCCCCCTGGTGGGCCCACCAAGCCCCTACCCCTAATTCCTAGACCTTTAACCCCCTCCCACTCCATCCCACGCCATTGCATC
CCTCCTGTTTCTGGCTTCCTGCTTTGCTCTAGCCTGTCCTCTCCTCAGTCCTCTGCCCTTCCCTGCCTGGTCCTCCCCCTGTGTCCTTCCTTAC
GTCCCTCTCGGGACAGGCAAAGCCCTACATCACCAGGGTTCTGTCCCGGGGGCTGCATGTCAAAGAGGTGAACTGTCCACAGGAAACCGCCTGT
ACGTCCACCCGGACTCCCCCAACACAGGAGCGCACTGGATGCGCCACGAAGTTTCATTTGGGAAACTAAAGCTCACAAACAACAAGGGGGCGTC
CAACAATGTGACCCAGGTAGGACCTGCTCTTCAAAACGTAGCCTCGCCCTGCTCCCCACCCTGGGTCTGAGACCTCCAAGGCCACAAGGGTCCT
GCGGGGCCAGTTTGGTTCATTTTTTCTTCCTTCCTACAGGAAGGAAGGTTCGTTTTTCTTCTGTCCTAAACGAAGGGTCATTTTTCCTCCTTCC
TAAACTCTGGCTGTTTCTCACCCACCTTGGGAGGAAGATGAGATGGGATGAAGCTGAATCTTGGACGGCGGTCATATTCAGCCCACACAGCCCA
ACCCAGCCTCAGGGTTTCTTCACCAAATCATGGGTTGCCATTGCTCCGGCACCATGAAAGGCACAGACTTGCCCCTGTGGTCCCAGGGAATAGA
TGCCGAGTTTCTCTAGGTTGAACAATCCTGTTAAAAGTGCCCTTGCTGGCTGGGTGTGGTGGCTCACACCCATAATCCCAGCACTTTGGGAGGC
CAAGGCGGGTGGATCACAAGCTCAGGACTTCAAGACCAGCCTGGCCAAGATGATGAAACCCCGTCTCTACTAAAAATACAAAAATTAGCCAAGC
ATGGTGGCGGCTGCCTGTAATCCCAGCTACTCAAGAGGCGGACGCAGGAGAATCGCTTGAACCCGGGAGGCAGAGGTTACAGTGAGCTGAGATC
ATGCCATTGCACTCCAGCCTGGGTGACAGAGTGAGACTCCGTCTCAAAAAAAAAAAAAAAAGAAAAAAGAAAAAAGAAAAAAAAGTGCCCTTGC
CCTAAAGCCCTGTTTGTGCTGATACCTTGGTCTCAATTGACATCTGGAGGTAGAAGAGGGGGCAAGAGGCTAGAATTGGTGCCCCAAAGAGTCC
GGACACTGGAGTTCGAAAGCTTGGGGGAAGTTACATGGTGGCAGAGCAGGGGAATGGACAGGATGACCTCAAGGTGCTTGCTAGCCTCACGTGG
GGCCAGCTGTTGGTGGGGGTGATGGGATCCTCGAAATCCTTCCTCCCCACCCCTACAACCCTCTTGCTCTGTCTACAGATGATTGTGCTCCAGT
CCCTCCATAACTACCAGCCCCGGCTGCATATCGTTGAGGTGAACGACGGAGAGCCAGAGGCAGCCTGCAACGCTTCCAACACGCATATCTTTAC
TTTCCAAGAAACCCAGTTCATTGCCGTGACTGCCTACCAGAATGCCGAGGTGAGGGCTGCCTGAGCCCCGGTGGGGAGGAGGGCAGAGTGGGGC
CCACTGTCTTCCTTGGGAGGGATTTGGAAAGTTCCCGAGCCCCAGACTCAGGACTCAGGTGACTCTATTTCCCTTCTCTCTAGATTACTCAGCT
CAAAATTCATAATAACCCCTTTGCCAAAGGATTCCGGGAGAACTTTGAGTCGTAAGTGCCACTGGGTTCAACTCAGCTTTGGTCCCTCCTGAGA
CACATCCTCTCCCTGCCCCTGAAAACAGGAGGGTGGGGCACACATCCTACACGTCGGCAGGCCAGGGAAGGAGGGTCGCAGAAGCAATGTCTCA
AACAGGTAGGCTCACAGGTGACTGGTTCTGCTTGTGACCCGTTTTCTTGCCTTCTATTTTTTTCTAGCATCTACACATCTCTTGACACCAGCAT
CCCCTCCCCGCCTGGACCCAACTGTCAATTCCTTGGGGGAGATCACTACTCTCCTCTCCTACCCAACCAGTATCCTGTTCCCAGCCGCTTCTAC
CCCGACCTTCCTGGCCAGGCGAAGGATGTGGTTCCCCAGGCTTACTGGCTGGGGGCCCCCCGGGACCACAGCTATGAGGCTGAGTTTCGAGCAG
TCAGCATGAAGCCTGCATTCTTGCCCTCTGCCCCTGGGCCCACCATGTCCTACTACCGAGGCCAGGAGGTCCTGGCACCTGGAGCTGGCTGCCC
TGTGGCACCCCAGTACCCTCCCAAGATGGGCCCGGCCAGCTGGTTCCGCCCTATGCGGACTCTGCCCATGGAACCCGGCCCTGGAGGCTCAGAG
GGACGGGGACCACAGGACCAGGGTCCCCCCTTGGTGTGGACTGAGATTGCCCCCATCCGGCCGGAATCCAGTGATTCAGGACTGGGCGAAGGAG
ACTCTAAGAGGAGGCCCGTGTCCCCCTATCCTTCCAGTGGTGACAGCTCCTCCCCTGCTGGGGCCCCTTCTCCTTTTGATAAGGAAGCTGAAGG
ACAGTTTTATAACTATTTTCCCAACTGAGCAGATGACATGATGAAAGGAACAGAAACAGTGTTATTAGGTTGGAGGACACCGACTAATTTGGGA
AACGGATGAAGGACTGAGAAGGCCCCCGCTCCCTCTGGCCCTTCTCTCTTTAGTAGTTGCTTGGGGAAGTGGGGCTCAAGAAGGATTTTGGGGT
TCACCAGATGCTTCCTGGCCCACGATGAAACCTGAGAGGGGTGTCCCCTTGCCCCATCCTCTGCCCTAACTACAGTCGTTTACCTGGTGCTGCG
TCTTGCTTTTGGTTTCCAGCTGGAGAAAACAACACAAGAAACTCTTGCGCATGAAGCAGCTTTTTGCATCTAGTGCGTGGGAGGGGTCAGGTGT
GGGACATGGGAGCAGGAGACTCCACTTTCTTCCTTTGTACAGTAACTTTCAACCTTTTCGTTGGCATGTGTGTTAATCCCTGATCCAAAAAGAA
CAAATACACGTATGTTATAACCATCACCCCGCCAGGGTCAGGGAAAGGACTCACCTCACTTTCGACACCTGGCCTGGGCTCCCCCTGCTCAAAC
ACACTCCGGATCAGACAAAAGGGGCTGGAAAGGGGGGAATGGCCCACATCTCAAGAAGCAAGATATTCTTTGTCGTGGTTGTGTGTGGGTGTGT
GTTTTTTCTTTTTCTTTCTTTTTATTTTTTTTGAATGGGGGAGGCTATTTATTGTACTGAGAGTGGTGTCTGGATATATTCCTTTTGTCTTCAT
CACTTTCTGAAAATAAACATAAAACTGTTGAATGTGCCTGCCTCAGTGCCAGCATGGGGGGACATGGATGGGGACTCAGTTGGGGTTGTACCCA
AGCTGGTGTACCCAAGGTGTTCTGTCAGCTTTCATTTATGGGGAACCTGCTAAGACCCTGAAATGACTCCAGCTGAGTTACAGCAAGGCCACAT
GTCCTACCTTCAGCACTCAGGGGGTTGGTTGATGCTACCTCTTAAGGCATCTTGGGACGGACAGAGAAGAATCCCTTGCCCTGTGTGCACCCTG
ACATTGAAAGGAGGGGTGTGAGGGCAAGGCCAAGGGCTGGACTGGGAGCGGGGGTGCAGGGCGCTGTGAGGCGGTGGCACTTGATTTTTCTTTG
CATTTCTAGCAGCCCTCCACCTTTATCTCTAGGGTTATTCAAGGATTAAAGAAATAAATATAAAATGAGCCATGTGAAATTACCATTTTTGTAG
GTCGAAAAGCCAAATATTAGCAATTTTATGTGACTCAAGCTAATACATGTAAAGGGTTTAAGAACATTGCCTGACAAACAGTAAGCACTCACTG
TGTAAGCTACTGTTACCAACAGTTTCTAGCTGTTTCTGTCTGTCTTTTTATACACACTGAATTGTGTTTGTAAAATAATACATACTTTTTTTTT
TTTTTTTGAGACAGAGTTTTGCTCTTGTTGCCCAGGCTGGAATGCAATGGTGCGATCTCAGCTCACTGAAACCTTCGCCTCCCAGGTTCAAGTG
ATTCTCCTGCCTCACCCTCCTGAGTAGCTGGGATTACAGATGTGCACCACCATGCCTGGCTAATTTTTGTATTTTTAGTAGAGACGGTGTTTCG
CCATATTGGCCAGGCTGGTCTCGAACTCCTGACGTCAGGTGATCTACCCACCTTGGCTTCCCAAAGTGCTGGGATTACAGGCGTGCGTCACCAC
ACTCAGCCTATACATGCTTCTTTTAAATAATTCAAGCAAGGAAGAAAAGTATAAAGACAACAATAAATTATCTCAAATCTTACCCATCAAGAAT
TATCATTAACATTAGGTGGGTTAGACAGAAATAGTTTTATAAATTGGAACCATACTGAAAAGGCTTTTTCTTAATGAAAATGCTTAAATTTTAG
CTTATAGAATTTAGTCACACACACACACACACACACACACACACACATCAAGACAAGCAAAACCTCTCAAACTCTCAAGTGAAATGAAGGGAGT
TGCTAAACTTAAGATAAATTTTTCTTCACTACAAGAAATATTTTCTTGGTTTTTTTTTTTTTTGAGACAGAGTCTCGCTCTGTTGCCCAGGCTG
CAGTGCAGTCGCACGATCTCAGCTCACTGCAACCTCCACCTCCTGGCTTCAACCACTTCTCCTGCCTCACCCTCCCCAGTAGCTGAGACTACAC
GCGTGTGCCACCACGCCCGGCTTTTTTTGTGTTTTTGGTAGAGACGGGGTTTTCACCATATTAGCCAGGATGGTCTCGATCTCTTGACCTCGTG
ATCTGTCTGCCTCGGCCTCCCAAAGTGCTAGGATTATAGGCGTGAGCCACCGTGCCCAGGCAAGAAATACTTTCTAAAAGAACACTCTCAGGCT
AAGACAGGTGCTTATAGGAAACATTAAGAAGTTACAGTTACTATGTTGCCCCACCACAGTCCAGGGTGCACCCCATTCTAATGGGGAAACATCA
TCATGGGCTTTGGGTAGTTCTCCCAGTTCTGTTTAAGGTCTGGAAAACCTGTTTTTTTTTTTTTTTTTTTTTTGGTCACAGTTCACATCCCATC
CTTTTTTGCTCTCTACTCAGAGGCACCTTCTAGTTAGAAGGAAAAAATACGGTGGCATACTTGGCTCCCTTTCCTTTGAATTTCGAATCTCATA
ATTTTGTAAATGACAGGAGATCTGCCACATTGTGCTCTGTGAGTTCAGATTATTTGTCCCCCTACTCCTACCCCGTGGCTGTCACTTTCTAAAC
TTAATGGGTTGCGTCTATTTCCTTTCTTGGAGGAACTGTGGTTGATAATTATAATACTCTTTTCGCTCACCATTTAAAAATGATTTTTGCTCCC
TTCATTAGATATCAAGTTAAGTGACTTGTGATCAAGCCATACTTTACCATCTTTTCTAGAAGTCTGACTGCATGTTTTGATGTGGTGTTATTTG
ACTCATAAAGGTTCATTACTATTATTTCTTCACTATTAGATGTACCTTTTACTGTATATACTGTGTATATATACACATACCACATATATCTATA
ATATATATAACCTAAATACCACACAACTCAAGATATTTCTTCAAGATTTAAACACCAATAATATCACAACTATATTTCCTCCATCTAATCTCCT
GCCTTTCCCTCATAGATAACCAGTATCTTTAATTTTGTGTTTTGCTTTTCTTGCCTTTTATTTGTTATATTTTTTATTTTTATTTTTGTAGAGA
TAGGGTCTTGCTATGTTGCCCAGGCTGGTCTTGAACTCTTGGGCTCAAGAGATCCTCCCACCTTGGCCTTCCAAAGTGCTGAGATTACAGTCAT
GAGCCATTGCATCCAGCCCCTTTCTTGCTTCTTAAAATTAAGTGTTAACACTTTATGTAAGTCTAACTACTATGCTATGTAGTTTTACTTGTTT
TTAACTTTATAAAATAATATATATGTGTGTGTGTGTATGTATACATACACACATATATGTTTTGATTTTTGACTCAACACTGTTTTTAAGATTC
ATCCATTTGTGGCAGGTAGCTATCTATTTCATTCATTTTTCACTATTTTATGATATTCCACTATGTGTATGTTTGCTATAATTTACTTATCTAT
TTTCCTGTTGACAATTTACACATTTTTGTTATTATGAACAGTTTTTAAAATATTTTTAATGTGTCTTCAGCCACACAAGTGCTAAAATTTCTCA
AGTCAAGTCTGTATCTAGAAATGGGTAGGGTATGTGAATGTACAATTTAACAAGAGAATGCCAAATTACTTTACCAATTTACACTCTCACCAGT
AATGTACAAGAGAACATGTTGAGTTTCTTTCTTTCTTTCAGCAGTAGTAAGGTTGAGCCTTTCTTCATATCTTTTCTAATCATAGGTTTGTAAA
ATGATTTGTTCATATCTTTCGCTCATATTGCTATTAGAGTATTTGTCTTTTTCTCATTGGTTCTGGTTCTTTATATATTCTTGACTTGATATCA
ACCATTATTTGGTTATTTATATTGCAATTATCTTCCTCTAGATTATTGTTCCTCTTTTCATTTTCTTTAAAGTGGTTTTCTGATGAACAAAAAT
TTGTAGTTTTAATGTAGCTAAATTTAACAATTTTACTTTTTTTTTTTTTTTTTAAGAGACAGGGTCTTGTTCTGTCACCCAGGCTAGAATGCAG
TGGTGTCATTATAGCTCACTGCAGCTGCAAACTCCTGGCCTCAAGTGATCCTTCCACTCAGCCTCCCCAGTAGTTAGGACTACAGGCATATGCC
ACCATGCATGGCTAATTTAAATTTTTTCTGTAGCGATGAGGTCTCGCTATGTCACCCAAGCTCATCTTGAACTCCTGACCTCAAGTGACCCTCT
CGCCTTGACCTCCCAAAGTGTTGGGGTGGCATGAGCCACCACACCTGGCCAACATTTTATAGTTAGTACTTTTCCTTGTCTCATTTAAGAAAAC
TTTTTCTGCTTGAGGGTTAGAAAGATATTCATCTGTATTTTTTTTTCCATAGGATTTGAACTTTTGCATTTGACATTTAATTCCTTTTACTGTT
TGGTGTGAACCATTCATTTTTGTTTTCGTTTTCCAATTTTTCCTGTTTGTTTTTGTTGAATAGTCTTTCTTTTGCCCATGGTAGTAGTCTCACA
TACTACTTCTGGTAGAGCAACGTCCTCTTTTTAAACTTTCTTCTTCTGAAGTGCTTTGACTATGCCTAGCTCTTTGCCCTTCTATATGATATAT
ATAATAACTTGGAAATTTTCATAACAACCAGACTGAGATTTTTATTGAGGTTGGACTAAATCTATTGTTCGATTTGAGAGAAGAGATAACTTTG
TGATATTGTCTTTCTATGCATGAACATGGGATATTTCTTCATTTATTTAGGTCATCTTTAAGGTTCTTTAGCAAAGTTTTCCAATTTGTAGGTG
TAGGACTTTTATATCTTTTGTTAAATTTATTCTTGGGTATTTCTTTTTTTCTCTGTAAAAGGCATATTTTAAAATATATGTTTCCTAGCTGTTT
GTTACTAGTTTGCAGAATTACAATTGATTTTTGTATATTGATCATGAGATCCAGCCATGTTGCTAAAGTCTTTTATTATTTTGGGGGAATTTCC
TATAACTTCTTTAGAGCTATCTCTGTCGCCAATCATATTATTTGAAAAAATGAGAGTTTTAGCCGCGCATGACGGCGGGCGCCTGTAGTCCCAG
CTACTCAGGAGGCTGAGGCAGGAGAATGGCATGAACCCGGGAGGCGGAGCTTGCAGTGAGCCAAGATCGCACCGCTGCACTACAGCCTGGGTGA
CAGAGCGAGACTCTGTCTCAAAAAAAAAAAAAAAGAGAGTAAAAAAAATGAGACTTTTATTTCCTTCTTTCTAATCATTATGCTTCTAATTTAT
TTTCCTTATCTTATTCGACTGGTCATAACCTCAAGTGCTGGTTTTTTGTCTTTTGGGGGATGGTTTCTTTCACTATTTTTTGACCCCACCTCTG
ATGGGGTCTCACTCCGTCACTCAGGTTGGAGTACAATGGTGTGATCATAGCTCACTGCAGCATTGAACTTCTGGGTTCAAGTGATGCTTCTGCC
TCAGCCTCCCAAATAGCTAGGACTACAGATGTGCACATTATGAAGCCTAGCTAATTAAAATTTTTTTTTTTTTTTTTTTTTTTGTAGAAACAGG
GCCTAGTTATATTGTCCAGGCTGCTCTTCAACTCTTGGCTTCAAGTGATCCTCCTGCATTGGCCTCCCAAAGCACTGGGATTAGAAGAGTGAGC
TGCCATGCTCAGCTCAGTTTTCCCCTATTTATTACACTTTTTTTTCCTTTTACTTGTTTGCATTTCTATAATTTATTTATTTTAGTGGCTTATC
TATACATTTTAATAAGTATATTTAATAATGAATGATGTTTTATCCTCCCTTCTCGTCAGTAAGATAATTTTAGATCACTTTAATTCTGATCTTC
CTCCTTCTGACTTACTGTTCCTCCTTCTGACTTACTAATAAAAACTTACTTCCTTCTTCTGACTTATTGATAACTCTTATATTATCATTTTAGT
AGCCATTCTAATAACCTTTTTTCATTCATGTTTTTCTCCTCTCAACTTACTCATTTCAGTTCTTTCTTTTTTTAACCCCTAAAAATTAGACTCC
TTTATTATTTTATTGAGACTATGCTTTGTCTGGACTTACCTTTATGTTACCATTCTTTTTGTTCATTTTCCCTTTTTTGCATTTCTCTTTTTTG
GGGGATCATTTACCTATATCTTTAAATCCTTTATAAGTTCTTTTAGAGAAGTTCTCTTTGTAAACTTCCCCTGGGCTTGTTTCTTATAAATGTT
ATCATTTTCCCTATTGGTATTGAGTGATTGTTTTGCTGAATACACAATTTTGGGTGAATAGTATTTTATTTATTTATTTTTTTTTTTTTTTGAG
GTGAAGTCGTACTCATTGTGTCACCCAGGTTGAAGTGCAGTGGCACGATCTCGGCTCACTGCAACCTCTGCCTCCCAGGTTCAAGTCATTCTCC
TGACTCAGCCTCCTGAGTAGCTGGGATTACAGGCATCCACCACCATGTCTGGCTAATTTTTTTTATTTTTTTTTTTATTTTTAGTAGAGATGGG
GTTTCACCATGTTGGCCAGGCTGGTCTTGAACTCCCGACCTCAAGTGATCTGCTCGCCTTGGCCTCCTGAAGTGCTGGGATTACAGGCATGAGC
CACTGCACCCAGCCTTTTCTTATCATTTTATATGAGTAATTCCACTGTCTTCTGGCTTCATTGTTGTGAGATCTGCTGCTATTCTAATTATCAT
TCTTTGTTAGGTCTTCTGTGTTTTTTCTCTGCTTTCCAGATCTTCTATTTATATTCATGCCCTAAAGATTCACTACCATGTGAGAAGGCATGAT
TTTATTTTTCCTTATCCTATTTGGGATACATTGTGTTTCATGGATCTTTGCATTCATCATTTTCATGAGCTCTGAAAATTCTCAGTCATCATCT
CTTTAAATATTGCTTCTTTCTATTCTCTCCAATTTCATCCTTTAGGACTCCAATCAGATGGGTGTCTTTTTTTTTTTTTTTTTTGACAGGGTCT
GGCTCTGTTGCCCAGGCTGGAGTGCGGTGGCACTTTCTTGGCTCACTGCAACCTCCACCTCCTGGGCTTAAGCCATCCTCCCACCTCAACCTCC
TGAGTAGCTGGGACTACAGGCATGCACGACCATACCTAGCTAATTAAAAGAAATTTTTTGTGTGTGTGTGGAGACAGGGTCTCGCTATGTTGCC
TAGGTTGGTCTTAAACTCTGGGCTCAAGCCATCTTCCCACTTTGCTTTCCCAAAGTGCTGGGATTACAGGTGTGAACCATGTTTATCCTTATAA
TTGCATTATTTGTTTTTGTAAACATTCTATACCTAGCTATTCTGTATTTTCAACATCTGCTGTCCTTGTAGGGGGTGATGATAGGGTTGTTGTC
TAAATCTGTTGTTTCTTCTGATTCTCAATGATAGTGGGTTTGCCTTCTTCCTTGCTTGGTGGTCTTCTTTCTTTCTTTATTAAATTTTCCTAAA
GAAAATCTTCAAGGAGTCTTTAGGCAATAACACTTCTGAGTATTTAACATGTCTTTATTTTACTTACACATGTGAGTGATAGCTTGACCCAGTA
TAAAAGTCTAGGTTGAAATCACTTTCTCTCAGAATTTTTTTTCTTAAAGACCAGTATCATAGTACTCTCAGATTTTTTTTTTTCCTGAGATGGA
GTCTCAGTCGCTCTGTTGCCCAGGCAAGAGTGCAATGGTGCCCTCTCGGCTCACTGCAACCACTGCCTCCTGGGTTCAAGTGATTCTCCTGCCT
CAGCCACTGGAGTAGCTGGGATTAGAGGTGTACGCCACCACGCCTGGCTAATTTTTATATTTTTAGTAGAGATGCGGTTTCACCATGCTGGCCA
GGCTGGTCTCGAACTCCTGGCTTCAAGTGACCCACCTGCCTTGGCCTCCCAGAGTGCTGGGATTACAGGCATGAACCATCGCACCTGCCCTACT
TACAGAATTTTTAAACCATTTTTTTCATTGTCACTTAGTTTTTAGTATTGCTCTTAAGAAGTCTGATGCCATTTTGATTCATGATCCTTTTTGG
TAGGTTTTTAGATCCTCTCTTGCTTCCCAGTGTTTTAAACATTCTGTATTATATACCTTGGTCTGAAGCCCTTTTTCATTTGCTATTCTGGCAC
ACCCAATGAGAACTATCAACCCAGAAACGCTGTTCTTCATTCTTAGAAAGTGTCTCTTCCTTGCTGGTTCAGTGGCTCATGCCTGTAATCCCAG
CACTTTGGGAGGGTGAGGCGAGCAGATCACTTGAGGTCAGGAGTTCAAGACCAGCCTGGCCAACGTGGCAAAACTCTGTCTCTACAAAAGTACA
AAAATTAGCCGGGCGTGGTGATGCATGCCTGTAATCCCAGTCACTCAGGAGGCTGAGGCAGGAGAATCGCTTGAACCCAGGAGGCGGAAGTTCC
AGTGAGCCAAGATTGCACCATTGCACTCCAGCCTGGGCGAATGAGTGAGACACTGTCTCAAAAAGAAAGAAAGTTTCTCTTCCTTTTTAAAAAA
CATTTTTCATATTTTTAATTTTTTTTTTTTTTTTTTTTTTTTTTAGAGATGAAGTCTCACTATGTTGGTCAGGTTGGTCTTGAACTCCTGGCCA
AAAAATTCTTCAACCAAGAGCATACTTCCTCCCAGAGAGCTGGTTTACTACCACACCACTGGGAGCTGATTTATTAGGCTGGGGCCCCCACAAT
GTTAGTGTGTAGAGATATGAGTTCTCCTAAAGAGACTTTTCCAAATTTCTTACCAGCTGACTGGAGAATATTGTAGGAGTGAAATATGAGAAGA
GAAACCGAGGGGCTAATTTTTTTCTTTTGCAAGCTTTCACTTACATTTTCTTCTTCCAGCTTTGCCTCACCCTTCCTAGACACTGTCTTTGGTA
TCCCCAAGTCCCATGTCTTTTTCTTTCATTTTCTTTAGAATGACACTCTATACTTCTAGGTGGTAGTGGTAGTGAAATAGTTATTTTGTTGCTC
AGGGCGGGAAGCAGTCATGGGCATCTTGATACATCTGATTTCAGACTTTTAGCCAATCCACCAATTGTCAGTACCATGCCTCAATCCCAGCTTC
CACTGTGCCAAGTTCTGAGCCTCTTAAGCATTCTTTCGGGCTAAATCAGTTCACTCATTGTTAATCCTGTTTGTAGACACTGTGGGCTATAGCT
CCATGTTGCTTAATCATGCACTACTCCTCCGTCTGCTTTCTAGTTTCCAAAAACTTGTTGAAATGTATTGTCTGGTTCCTTTTCCTCTGCTGTT
CTCTTTGTTCCTGTGGTTTCATACATTTGAAAAATTCCTATATTATCATTTTAGTAACCAATATACAATTTTTTTTTTTTGAGACAGAGTCTCG
CTCTGTCGCCCAGACTAGAGTGCAGTGGTGCAATCTCGGCTCACTGCAGCCTCTGCCTCCTGAATTCAAGCGATTCTCCTGCCTCAGCCTCCTG
AGTAGTTGGGATTACAGGTGCGCACCACCATGCCCGGCTAATTTTTGTAGTTTTAGCTGAGACGGGGTTTCACCATGTTGGTCAGGCTGGTCTC
GAACTCCTGACCTTGTCATCTGCCCGCCTCGGCCTCCCAAAGTGCTGGGATTACAGGCATGAGCCACCGCGCCCGGCCCAAATATACAAATTTT
AACCTAAACACTCTTCATCAATTTTGAATGGAACTTGGTACATTTTTTCAGCCTTCATGGATTGGCCTTCTTCTTTCTCTACCCCACACCCCCA
AATTTCAGACAAGTTTTCTTCTATTTTGTCTCCGATTACCTCCTATCCAGCTAGTATTTATTTTTCAGAGACATTCATTACTCTTCGACTAGAT
CTCAACCCTCTGTTTCTCTCCATTTTCTTTCTTTCTTTCTTTCTTTTTTTTTTTTTTTGAGACAGAGTCTCACTCTGTTGCCCAGGCTGGAGTG
CAGTGGAGTGCCGCGATCTCGGCTCACTGCAAGCTCTGCCTCCAGGGTTCACACCATTCTCCTGCCTCACCCTCCCAAGTAGCTGGAATCACAG
GCTCCCGCCACGCCACCACACCTGGCTAATTTTTTGTATTTTTAGTAGAGATGGGGTTTCACCATGTTAGCCAGGATGGTCTCCATCTCCTGAC
CTTGTGATCCACCTGCCTTGGCCTCCTAAAGTCCTGGGATTACACGCGTGAGCCACCACGCCCGACATTTTTTTTTTTTTTTTTTTTTGAGACA
GGATCTCACTCCTGTTGCCCAGGCTCGGGTGCAGTGGCGCTATCACAGCTCACTGCCACCTCGACTTCCCAGGCTCAGGTGATTCTCCCACCTC
AGCCTCCTAAGTAGCTGGGATTACAGGCACATGCCACCATGCCCAGCTAAATTTTTGTGTTTTTAGTAGGGACAAAATTTCATCATTTTACCTA
GGCTGGTCTCAAATTCCTGGGCTCAAGCAATCTGCCCGCCTTGGCCTCTCTAAATGTTGAGATTACAGGCGTGTGCCACTGCACCCGGCCTTTG
TTTCTCTTCCTATTACTTAGTTTTCATTTTTTGACTTTATATTCTGTCCTCTGGGAGAGATTTCCAAAGTTATCTTAGTAGTGGCATATTAACT
TGGGGATGGCTCAAGATTAATTTCTAAGCAGAGAGTGATCCAGAAGGATAAATATCTGTACTTATGATTTACATGAATGCCTTTCTGAGTGTCT
GTGGTTACAGCTATCTCTTAATATGCCATATTTTGTAAGAGTTTATATGGCAAGAGTTATAAACAGCT
HUMAN SEQUENCE - mRNA
CGGCCCGCTGGAGAGGAAGCCCGAGAGCTGCCGCGCGCCTGCCGGACGAGGGCGTAGAAGCCAGGCGTCAGAGCCCGGGCTCCGGTGGGGTCCC
CCACCCGGCCCTCGGGTCCCCCGCCCCCTGCTCCCTGCCCATCCCAGCCCACGCGACCCTCTCGCGCGCGGAGGGGCGGGTCCTCGACGGCTAC
GGGAAGGTGCCAGCCCGCCCCGGATGGGCATCGTGGAGCCGGGTTGCCGAGACATGCTGACGGGCACCGAGCCGATGCCGGGGAGCGACGAGGG
CCGGGCGCCTGGCGCCGACCCGCAGCACCGCTACTTCTACCCGGAGCCGGGCGCGCAGGACGCGGACGAGCGTCGCGGGGGCGGCAGCCTGGGG
TCTCCCTACCCGGGGGGCGCCTTGGTGCCCGCCCCGCCGAGCCGCTTCCTTGGAGCCTACGCCTACCCGCCGCGACCCCAGGCGGCCGGCTTCC
CCGGCGCGGGCGAGTCCTTCCCGCCGCCCCCCCACGCCGAGGGCTACCAGCCGGGCGAGGGCTACGCCGCCCCGGACCCGCGCGCCGGGCTCTA
CCCGGGGCCGCGTCAGGACTACGCGCTACCCGCGCCACTCGAGGTGTCGGGGAAACTGAGGGTCGCCCTCAACAACCACCTGTTGTGGTCCAAG
TTTAATCAGCACCAGACAGAGATGATCATCACCAAGCAGGGACGGCGGATGTTCCCATTCCTGTCATTTACTGTGGCCGGGCTGGAGCCCACCA
GCCACTACAGGATGTTTGTGGACGTGGTCTTGGTGGACCAGCACCACTGGCGGTACCAGAGCGGCAAGTGCGTGCAGTGTGGAAAGGCCGAGGG
CAGCATGCCAGGAAACCGCCTGTACGTCCACCCGGACTCCCCCAACACAGGAGCGCACTGGATGCGCCAGGAACTTTCATTTGGGAAACTAAAG
CTCACAAACAACAAGGGGGCGTCCAACAATGTGACCCAGATGATTGTGCTCCAGTCCCTCCATAAGTACCAGCCCCGGCTGCATATCGTTGAGG
TGAACGACGGAGAGCCAGAGGCAGCCTGCAACGCTTCCAACACGCATATCTTTACTTTCCAAGAAACCCAGTTCATTGCCGTGACTGCCTACCA
GAATGCCGAGATTACTCAGCTGAAAATTGATAATAACCCCTTTGCCAAAGGATTCCGGGAGAACTTTGAGTCCATGTACACATCTGTTGACACC
AGCATCCCCTCCCCGCCTGGACCCAACTGTCAATTCCTTGGGGGAGATCACTACTCTCCTCTCCTACCCAACCAGTATCCTGTTCCCAGCCGCT
TCTACCCCGACCTTCCTGGCCAGGCGAAGGATGTGGTTCCCCAGGCTTACTGGCTGGGGGCCCCCCGGGACCACAGCTATGAGGCTGAGTTTCG
AGCAGTCAGCATGAAGCCTCCATTCTTGCCCTCTGCCCCTGGGCCCACCATGTCCTACTACCGAGGCCAGGAGGTCCTGGCACCTGGAGCTGGC
TGGCCTGTGGCACCCCAGTACCCTCCCAAGATGGGCCCGGCCAGCTGGTTCCGCCCTATGCGGACTCTGCCCATGGAACCCGGCCCTGGAGGCT
CAGAGGGACGGGGACCAGAGGACCAGGGTCCCCCCTTGGTGTGGACTGAGATTGCCCCCATCCGGCCGGAATCCAGTGATTCAGGACTGGGCGA
AGGAGACTCTAAGAGGAGGCGCGTGTCCCCCTATCCTTCCAGTGGTGACAGCTCCTCCCCTGCTGGGGCCCCTTCTCCTTTTGATAAGGAAGCT
GAAGGACAGTTTTATAACTATTTTCCCAACTGAGCAGATGACATGATGAAAGGAACAGAAACAGTGTTATTAGGTTGGAGGACACCGACTAATT
TGGGAAACGGATGAAGGACTGAGAAGGCCCCCGCTCCCTCTGGCCCTTCTCTGTTTAGTAGTTGGTTGGGGAAGTGGGGCTCAAGAAGGATTTT
GGGGTTCACCAGATGCTTCCTGGCCCACGATGAAACCTGAGAGGGGTGTCCCCTTGCCCCATCCTCTGCCCTAACTACAGTCGTTTACCTGGTG
CTGCGTCTTGCTTTTGGTTTCCAGCTGGAGAAAAGAAGACAAGAAAGTCTTGGGCATGAAGGAGCTTTTTGCATCTAGTGGGTGGGAGGGGTCA
GGTGTGGGACATGGGAGCAGGAGACTCCACTTTCTTCCTTTGTACAGTAACTTTCAACCTTTTCGTTGGCATGTGTGTTAATCCCTGATCCAAA
AAGAACAAATACACGTATGTTATAACCATCAGCCCGCCAGGGTCAGGGAAAGGACTCACCTGACTTTGGACAGCTGGCCTGGGCTCCCCCTGCT
CAAACACAGTGGGGATCAGAGAAAAGGGGCTGGAAAGGGGGGAATGGCCCACATCTCAAGAAGCAAGATATTGTTTGTGGTGGTTGTGTGTGGG
TGTGTGTTTTTTCTTTTTCTTTCTTTTTATTTTTTTTGAATGGGGGAGGCTATTTATTGTACTGAGAGTGGTGTCTGGATATATTCCTTTTGTC
TTCATCACTTTCTGAAAATAAACATAAAACTGTTAAAAAAAAAAAAAAAAA
HUMAN SEQUENCE - CODING
ATGGGCATCGTGGAGCCGGGTTGCGGAGACATGCTGACGGGCACCGAGCCGATGCCGGGGAGCGACGAGGGCCGGGCGCCTGGCGCCGACCCGC
AGCACCGCTACTTCTACCCGGAGCCGGGCGCGCAGGACGCGGACGAGCGTCGCGGGGGCGGCAGCCTGGGGTCTCCCTACCCGGGGGGCGCCTT
GGTGCCCGCCCCGCCGAGCCGCTTCCTTGGAGCCTACGCCTACCCGCCGCGACCCCAGGCGGCCGGCTTCCCCGGCGCGGGCGAGTCCTTCCCG
CCGCCCGCGGACGCCGAGGGCTACCAGCCGGGCGAGGGCTACGCCGCCCCGGACCCGCGCGCCGGGCTCTACCCGGGGCCGCGTGAGGACTACG
CGCTACCCGCGGGACTGGAGGTGTCGGGGAAACTGAGCGTCGCGCTCAACAACCACCTGTTGTGGTCCAAGTTTAATCAGCACCAGACAGAGAT
GATCATCACCAAGCAGGGACGGCGGATGTTCCCATTCCTGTCATTTACTGTGGCCGGGCTGGAGCCCACCAGCCACTACAGGATGTTTGTGGAC
GTGGTCTTGGTGGACCAGCACCACTGGCGGTACCAGAGCGGCAAGTGGGTGCAGTGTGGAAAGGCCGAGGGCAGCATGCCAGGAAACCGCCTGT
ACGTCCACCCGGACTCCCCCAACACAGGAGCGCACTGGATGCGCCAGGAAGTTTCATTTGGGAAACTAAAGCTCACAAACAACAAGGGGGCGTC
CAACAATGTGACCCAGATGATTGTGCTCCAGTCCCTCCATAAGTACCAGCCCCGGCTGCATATCGTTGAGGTGAACGACGGAGAGCCAGAGGCA
GCCTGCAACGCTTCCAACACGCATATCTTTACTTTCCAAGAAACCCAGTTCATTGCCGTGACTGCCTACCAGAATGCCGAGATTACTCAGCTGA
AAATTGATAATAACCCCTTTGCCAAAGGATTCCGGGAGAACTTTGAGTCCATGTACACATCTGTTGACACCAGCATCCCCTCCCCGCCTGGACC
CAACTGTCAATTCCTTGGGGGAGATCACTACTCTCCTCTCCTACCCAACCAGTATCCTGTTCCCAGCCGCTTCTACCCCGACCTTCCTGGCCAG
GCGAAGGATGTGGTTCCCCAGGCTTACTGGCTGGGGGCCCCCCGGGACCACAGCTATGAGGCTGAGTTTCGAGCAGTCAGCATGAAGCCTGCAT
TCTTGCCCTCTGCCCCTGGGCCCACCATGTCCTACTACCGAGGCCAGGAGGTCCTGGCACCTGGAGCTGGCTGGCCTGTGGCACCCCAGTACCC
TCCCAAGATGGGCCCGGCCAGCTGGTTCCGCCCTATGCGGACTCTGCCCATGGAACCCGGCCCTGGAGGCTCAGAGGGACGGGGACCAGAGGAC
CAGGGTCCCCCCTTGGTGTGGACTGAGATTGCCCCCATCCGGCCGGAATCCAGTGATTCAGGACTGGGCGAAGGAGACTCTAAGAGGAGGCGCG
TGTCCCCCTATCCTTCCAGTGGTGACAGCTCCTCCCCTGCTGGGGCCCCTTCTCCTTTTGATAAGGAAGCTGAAGGACAGTTTTATAACTATTT
TCCCAACTGA

TABLE 27

MOUSE NOMENCLATURE
ICSGNM Stat5b
Celera mCG20224
HUMAN NOMENCLATURE
HGNC STAT5B
Celera hCG16577
MOUSE SEQUENCE - GENOMIC
CAGGGGCGGGCACTGCTGCTTGTCTCGTCACGATTCCTCGTGATTCATCTTAGCATGTCAAGTCTGAAAATGTAGAGTGCCACTCTGCCCGCCC
TATGGGTGGGGTTGCCCAGCCCCTCCGTTAGAGTGTTGTCCACCCGATTCACCTACTGATGTGCCTTTTCCTTTCTTTTTTTTGTTTTTGAGGT
CCAGGGAACTGATAGCAGGGCCTTGTGCCTTCTAGGCCAGAGCTCTACCAGTGGGCCGTGTCTCCAACTCCTTCTCATTCTGTGACTAGTAAGA
AGCGTGCAAAGCCAAGAACATCCTGTGCCCAGAGATGGATGTTATCTATGGCTTCTGCAGCTTTGATGCTCCTTGTCTGAGTCTACTGTGTGTC
TGTATGTGTCTGTGTGTGTCTCTGTGTGTGTGTCTGTGTGTATCTCTGTGTGTATCTCTGTGTGTGTCTCTGTGTGTGTCTGTGTGTGTGGGTA
TGTCTGTGTGTATCTGTGTGTGTGTCTCTGTGTGTGTCTGTGTGTATCTGTGTGTGTGTGTGTCTGTGTCTCTGTGTGTGTATCTCTTTGTGTG
TCTCTGTGTGTGTGTCTGTGTCTCTGTGTGTATATCTGTGTGTGTGTCTCCGTGTGTGTGTCTGTATCTCTGTGTATGTATCTCTGTGTGTGTC
TGTGTGTATGTGTATCTCTGTGTGTGTCTCTGTGTGTGTGTCTCTGTGTATCTCTGTGTGTGTCCAAGTGCACGCATGTATCTGCACAAGTCTA
TAGACGCCAGAGATCAATGTTGGGTGCCTTCCTCAATTGCTTTACAGCAGCAGTTCTCAACCTCTCTGGGGCTTGAACGACCCTTTCACCAAAG
CCCATGAGAAAACACCGATATTTATATTACAACGCGTAACAGTATCAAAACTACAGTTATCGAATAGCACAAAAATAGTTTTATGGTTGGGGGC
TCACCACAACATGAGGAACCATATTAAGGGTCGCAGCGTTAGGGAAGTTGAGACTGGGTATCTCAGTGAGCCTGCAGCTTGCCCACTCAGCTGT
GGGGGCCAGCAAGCTCCAAGGATCCTTCTGAATGCCTTCCCAGAACTGGGAGCACAAGTGCCTGCTGTTCTTTAACATGGGTCCTGGGAATTGA
ACTCAGATCCTAAAGACAGCACAGTGAGTGCTTTGCCAACCAGGAGCCGTCTCCCCAGCCCAGCCTGCTTTCTTTTTGAGGGAGCACGTGTTTT
AATATAAACATGGTTGTATTTAGGTGAGAAGTTTAAATATGCGAGAATCCACTGATCTGTGCTTACTCTCTGCACGCAGCAAATAGCCACCCAA
CCCCCGCGCCCCCCTTTTACCTTCTGTCCTGTCCTTCTGTTCATTACCTCTCTTCTTCCCAAAGCGACACACGGTGGGTGAAGATTTATGACTG
TCTTCCCAGAATTTTATTATCTGTGTTGCCCGGGCAAATTTTTTCCTTTTTTGTTTCTTTGTGGGCCACTTTCACTCCTGTTCCCTTCTCATGG
GCTTATGATTTTTTTTTTCTCCCCTGAAAGAGAATCTTCTTTCTGGTTGCTTATAAGCTAGTGGTTAGGTTGTTTTGTTTTGTTTTGTTTTGTT
TAAAGGGCAGATGGTGCTAGTATTGACCTACTTTTAGATGTGGTCATTTACCCTAACCTTGCTCGGTCAGGCCAGTATGAACACAGTCTTAATC
TTCTAACACTGTGTTTCATATTTTCTGTCTCTCAAAGTCCTTGTATTCGGTGACCACTCAACACAGTAATGATGTTGACATTGACAGTGAGAGC
ATCCACATGACCAGAGACCTGGTCCCTTATCCCCCTCCCAGGATCCCTCAGTTCTCAGCGGGTGCTGGGAGGTTTAACCATTTGCAATGCTGGC
ACCTTGCCTGGAACTTTTCAGATCAGAACCAATGTTTATCCATTGAGTCCATCTGCCAACTGTCCCTAGCCTTTGCTAAAACCAGAGTCTCCCA
ACACTGCGTTTCTCCTCTCTCTTTAAATTATTAAGTGTGACAACGTGTGCACAAGCACAGAGGGTGACATGACAACTCACCACAAGTTCTCAGG
ACACAGGCGGTGCTGGGCAGTGGCAACAGGGCACTGCACAGTCACGGCCTCTTGGCACTGAGAGATGGGGTCATCTTGGTAATGGCATTAAGGG
CATGTGCCACCAGGCCTAGCTTTTTTCTCTTTTTCAGTTACAGCTTTATTGAGATAGAATCACGTGTTATAAAATTCACCTTTTTATTTAGTTT
CGGCTGTGTGTGTCTGTCTGTCTGTACCATGGCATCCTTGTCGAGGTCAGATGACAGCTAATGGATGTTTGCTCTTCACTCCACTATGGGTTTC
AGGGGTCAAACTCAGGTCATCAGACTTGAATGACCAATGCTTTTACCTGAGAGCCATTTCCCTGGCCCCATCAACATTCTAAAATGTCCAGAAT
TCAGTCTCCCGGGCGTTGTAGTCCATTACCACTGTCCACCCCAGCCTTAATGTGTTTTCCTCACTTCCTCGCTTCCCCAAAAGTTCAACCTCAC
TCTTTTCTGTGGCTCTGAATTTGCCTATTGTAGAATCACAGTGTAGTTCAGGAAGTTTGTCCTCCACAGTTTTCAGTGTTCTCCTTCTGAGTTG
GGGGAACAGGACTCACAGTGCCCCTGCCCCACCCTTCCATAGGTTCTTGTCTGTAAAGCCCTTGTGTCAGCGGGACTGATTAAGAAATCAGAGT
ATCTGGGACTGGAGACATGACTTAGCAATTAAGAGCACTGGCTGCTCCTCCACAGATCCTGAGTTCAAGTCCCAGCCACCACAGGGAGGTTCAT
AGCCATCTATAATGTGATCTATACATAAAAAGGGAAGGAAGGAATGAAAGAAGGGAGGAAGGGAGGGAGGAAGGAAGGAAGAAGAGTATTAGGC
CCAGCAAAATGACTCAGTGGATAATGATGATTGCCACCAAGCCTGACATCCTGAGTTTGATCCCCAGCACAACACACAGTAGGAGAACAAACCC
CCTACAAGTTATTCTCTAACTGCCAAAGGCGTGCTATGGTGTGTGCGCACACACGTGCATACGCGTGCGCACACAAATGCAATAAAAATAAGAA
AAAGAATACCTTATTCTTAAAGGAAGGTAACTTTTGTCCTGTGGAGCATTAGGAAGCAGGAGGATAGCAGGAGCTGCTCATCAATCAGATGAAG
TTTGGAAAAGCTTGTTGGTGGGGTGAAGATGGAGGTGGGGAGAAGGAAGTGCTAAGTGTAAGTGTGAAAGAATCATTGGCTCTCCTCAGGGGCT
GACCACACTCAGGCTCTGTGTTACATACTTACACACACACACACACACAACTCAGAAGTTATAGAAACTCCGAATAGTAAAAACTAACCTGGCA
AACCATATCTCTCTGTGAGTGTTTTAAACCCTCATCTCTCCTCAAGCCACAATTTGTTCAGTCCATTGGCAGAGTTTCGCTGGAAGCTTCCAGC
AAGAGAGCCTGCTTTCCGCCAGGGAAGTTACAGCAAAACAGTCTCTGGTGTTCTGAAAACCTTCACAGTAATGCCTTTGTTTGCAAACGCTTCT
GCTTGGGGCTGGGAGTGTGCCTCAGTTGGTACAGCTCTCGACTAACAGGCACAAATTGACCCCCAGAACTAGACTAACAGAACACACCTATAAT
CTCAGTACACAGGAGGTGGAGCAGCAGGAGCAGGTATTGAACTACATAATGAGTCCAGTGCCAGAGGCAAGCCTGGGCTACCCAGAGCTGACCT
AAATATATTTGTTAAAGAAAAAGTTTCTGATAGAATGTTAGCTATTGATACAACATAACACTGATCACCAGCATAGTAGATATCCCTTCCTTTG
GTTTCCAATAAGCCTGTGAAGTTGAGGTTTTTATAAACAGGTACTTCTGGCCCCTTATTTAACCTTCTCCACCCTTGGTCTAGGTGTTCTGGGT
CAACATAGGTGCTGGGTGTGTTTAGCCGGTATAGCACTCTGTGTCCAGGCCCTGGCCAAGCTGTGAACTCCACAAGTGTGACTGCACAGAGCTC
GGCTGACTGTCAACACTGTCCTAAAAATATGATGCTGAATCCAGTTCCCAGGGTTTAGAGCGGCCCCTGTGATGATTTACCAGCGATACAAACC
ACTTCCCTTGTCAGAATGTCACGAACTGAAAACTGAGCTGCCGGGTCAATAAGCAGAGAAGAGCATGCGCATGCGCGGGGTAGGCTGTCTCCAT
GCGGGTGGAGGAGCCGCCCGGTGGCTGGATTGACCATGGGTACAGGGCCGGTGGGGGCCCCCCCACTTACACCTGTGTGCACAATGTTTCCTGG
TGAGTGTGGAGAGTTCTGACAGGCATGGGAGAAAAGCACTAACCAGACCACAGGTCTTGAGCATTTAAACTGGTAAGGAGTGGGAAGAGTTTTT
TGTCTGGAGTGAGTTACTTACCATTTAGGAAGTGGAGGCAAGGAGCTATAATGGCTACAACAGTAACCTTCATAAATCTATGTGGATAAAACCC
ACAGTTTAAAAGTAAAACGGACATCACTGTACTATTTAGTGTGGATATATATATACATTTTTTTTATTTTAACTGACACAACTGGGAAAGGGCA
TGCTCCCTCATACAGTGTGCGATGATCAGATCAGCTTAATTGTCAGATCTGTCACTCAACATTGATGATGTGTTTGTGCTGGGAGCGTTCAGAT
TCCTTCTCCTCGTGACGGATAGTGAGCAAAATCCACACCGGAGCTCTATTTTCTATCCCTATTAAAGTACTTTTATGTCGTGAAACTATGCTGT
ATTTTTCAAGATAAGGCAATTACCACTTTGTATTTACCAGCTATTATCCTTCAAATACTGTACTATTAAAAAAAAATACCATGACACGCTCAGA
ATTGGAAACATAAAAATATTCTGACCAAAAAATTAATATATATATTTTTTTTCTGACAAATTCTCAGAGCTCAGCATTCTACTCTTTAATAAAA
ACTAAGAACAAATATAAAACCTGAAACACAAATTGCCTTTATACCATGCAGCTCCCTAGAGTTCTTCTTCTGTTTCTGCAATATTGTTTTTACA
TCTTTTGAAGCTGGACACACCTTTACTACCCACATGAGTGTAAGTGTGAGGTAAAGATAGGTGGATTTCTGTGAGTTCAAGGTTAGCTGGTCTA
CATGTTAAGTTGCAGACCAGCCAGGTCTACTGAGTGAGACTCAGTCTCAAAAAATAAACAAAGGCGTTTGGGTTTTTTTTAGGTTTGGTTTGGT
TTGGTTTTTGTTTGTTTGTTTGTTTGTTTGTTTGAGATAAAGAGAGAGAGAGAGAGAGAGAGAGAGAGAGAGAGAGAACGTATCATCTTGTAAA
CCCAGGCTGGCCTTAAACCTTGACCTGTTTACCAAGTGCTGGGATTGCAGGTGTATACCACCCCATTAAGCTGTTTCTCTCTTAAAACAACAAT
AAACTCCGGAAATATGCTCAGCCAACAGGACAGCTAATAGATTTGTGTATTTGTTCATTTAGCCTATCGGTTCCCTACCAGTGTAGGTCACAAC
CCTTTTGGGGATTGAATGACTTTCTCACAGGGTCATCTAAGACCATCGGAAAACACAGATATTTACATTACAATTCATAACAGCAAAATTACAG
TTATGAAGGAGCAATGAAATAATTTTAGAGTTGGCATGACGAACTGTATTAAAGGGTTGTAGCATTAGGAAGAGTGAGAACCACAGATTTAGCA
AATACTTGTTTACTGCTCTCTGTGCTGGGCTGCTACACATGCATTTTGGGGACAGGGCAGTTAAGACATGTCCTCTTCTATTGCGGTTCAGTGG
AAGGAGACAAATTAAAACAAAAGAAAACTAGACAGAAAAATGACAGGCCTCTGTGATGAAAGAGAGATCACCCACATAGAGTCACCCCAGTTAT
ACAGTTATAGCTGAGGAGCTCATTAAAGATGTTTCATTCTACACTGTAGAATTTCTTCTTCTTTTAAAAAAAATTTGTTTGTTTGTTTGTTTAT
TATATGAGTACACTGTAGCTGTACAGATGGGTTGTGAGCCACCATGTGGTCACTGGGAATTGAACTCAGGACCTTTGGAAGAGCAGTCAGTGCT
CTTAACTGCTGAGCCATCTCTCCGGCCCTACACCGTAGAATTTCATACTCATGTTATGTTCTGTAATAACTTTGAAAAGAGCTCATCTGAAGCT
GGAGAGACGACTCAGTCGTTAAGAGGCTGCCCTTCCAGAGGACCTGGGTTGTATTCCCAGCAACCACATGGCAACTCACAACCATCTGTAACTC
CAGTTCTAGGAGATCTGACCCCCTCTTCTGGTCTCCACAGGTACTACACACATGTGGTCCACAGACCAACATGCAGGCAAACACCCACACATTA
AGTAGATCGGAAAAATATATATCTTAAAAAAGGAAAAAGCAGACATGTCCAATTGTGTTTGCTCATATTTGTTTATTAAAGCAACAACAACAGA
ATAGTACTGCTTATCTGCCAAGCACCTATGTACATAAAGTTATCGTGTGTTTAGTAAAGATGATGCATGCCAGACAGNNNNNNNNNNNNNNNNN
NNNTTGGTGTCTGTTGGAATCACTCAAATTTTCATTAAGGGAGCTACTGTGAGGCCCCACTTCCATCTTGTCCTAGGGCTGAGTGTGTGGGCGT
GCATATGTGGATCTGAGTCTGTGTGTGTGTGTGTGCGCGCGCGTGTGCATGTGCGTCTGTGTGTGTGTGTGTGTGTGTGTGTGTATACTTATGG
AGGCTTGAGCTATTCACCTCTTAATTTTATTTTTGAAACACGGTCTCACTCTGTTGCCCTGGCTGCCCTGAAACTTTCTATATAGACCAGGCTG
ACCCTCAAACTCATGAACAGCCACCTACCTCTGCCTTGTGACTATGACACCATACGCACCCCCTTCCCTCGATGTCTTTTGAGAGAGTTTCTAA
TCTCCTGGAGTTCCTCTCTCCATCTCCCAGCTCTGAGATTACAAGTGTCCACCAGGCTTTTTACTTGGATTCTACGATTTGAACTTGGGTCCTC
TTGCTTGCAAGGCAAACACTGGCTGAATCACCTTCCTAGCCCAGTCTGCATTTGTTTGTTTTCATATATTTATGTTGACTTGCTGTGTATGAAT
GTTCTGCACACATGTATATTTGTGTACTATGTGCATGCAGTACCCTGGAAGGCCAGAGCAGGGCATCAGATACTCTGGGTCTGGAGTTACAGAT
GGTTGTGAGCCATCAGGTGGGTGCTGGGAAACAAGCCCAGGTTCTCCTCAAGAGCAACAGTGCTCTTCACCACTGAGCCATCTCTCCAGCACCC
CCCTCCTTGTTCTTAACTAAGAAAGAGTGCTACCCGAGCCCCACTCTTCGTCTGTCAGGCCTCTGCATGCTTAAATCTCCAGTCTCCTCCACTC
CCAATCTCTTTACTCTTCCTCCAAAATCATGAGCTGCTCCTGGTCTGATTTCGAAACTTCACTAAAACATTGTACAGTTGTCTTCCAGATGTTT
GACTGGGATGCAGAATTGCTCTCCTGTTCCAGTTTTCCTGTGATGAACTCACACCCACTGAACTTTGCAGTATTGGAAAATCTCACTCTTATCT
CAGCCCTGGAGCCTGAAAGCTCCGAGTTAAACCATCCAAGTATCCTATAGCCGCACACACATCTAAAATAGGTAACCAACCGGCACCCACCCTA
CACCTCACTGCTAGCTAACTGACCTGCCCCATAAATACCTCAGTGTTACTCGATCCGACACCCACTGCTCACAGAGAGATCTTTGTTTCCTGGC
ACACATGTGCGTGCAGTCTCTGGGAGTCAACAAAACCCAGGTCCAGTGCACTGGACCATGGCTGTAACACAATCCTTAGGCCAACTGTGAAAAT
CAGTAGCTAAGTCATTTTGAGATTGGATTCAAGACGCCCAGATCCGTATTACCAAAAGCAGTGTTACACCAAGTCTGGTTTTAATGTCTGAAGT
TCCTATTAACATAGCTTCTCCACTCTGGGGAATCCCAGGTCTCTGCCAAATGCACTTAATTCTCAGGTCTGTCTGAAAGGGCTCCTAGTAGTTT
CAGAAAGCATCTATTTGTTTGTTTGTTTGTTTGCTTGCTTGCTTGCTTGCTTGTTTGTTTGCATACACTGTAGCTGTCTTCAGACACACTAGAT
CCCATGACAGACGGTTGTGAGCTATCATGAACTTAGGACCTCTGGAAGTATAGTCAGTGCTCTTAACTGCTGAGCCATCTCTCCAGCCCCCAGA
AAGCCATTTTCTAAAGCGTGTTTTTGTTGGTGCAGCTTGTTTTGGTTTTGATGTTGTTTCTTTAAGAAAGAGTCACAGGAAGCCCACCCTGGCC
TTGAATTCTTGATCCTCCTGCCTTCACATTCTGAGTTCTGGGATTAAAGGTTCTACCACAAGATCCTGTTAAAAAGAAAAAAAAAAAAACAGAT
AATCTCACTATTGCTGGGACTAGTGGCGCCAAGCCTGTAGTCTTAGGTTAAATTAGAAGACAGCAAGTTTAGTGGGCCATGGTGGAACAGGCCT
TTAATCCCAGCACTCGGAAGGCAGAGGCAGGCAGATCTCTGAGTTTGAGGCCAACCTGGTCTACAAAGTGAGTTCCAGGACAACCAAGAGAAAT
CCTGTCTCAAAAAATAAAACAACAACAAAAAAGAAATAATATAAAATGAATAAATAAATGAAATAAAATAAAATGATGATAAGTTGGGCTGGAG
AGATGGCTCAGCAGTTAAGAGCACTGACTGCTCTTCCAAAGGTCCTGAGTTCAAATCCCAGCAACCACATGGTGGCTCACAACCATCTGTAATG
AGATCTGATGCCCTCTTCTGGCATGTCTGAAGACAGCTACAGTGTCCTTACATATAACAATAAATAAATCTTTAAAAAAAAGATGAAAAGTTTA
AGGTCAGCCTGGGCTACAGAGCGAGTGCATAGTCAGCCTACACAACTTAGTGAGACACTGTCTCCAAATAGAAAGTCCAAAAGGGGCCTGGAAT
ACAGGTCAGCCATGGAGCTCTTGCCCAGCATGGCCCAGGTCATAGGTTCAATCTCCATTACAAAAGGAAACAACAAACCCTACAGAAAACCAGC
CCTGCCGCTCACCATTGTCACACAGCAATCCATTCTTCTCCTAGTCCACACATACTATTTCTGTGTTGAACTGTATGTATGTGCAAATTCCATA
GCAGTAACTCCAGAGGTGAAAGAACATTGGGGAGCTTGGCCTGATAAGCCTCTGCTCTGGGTAAGAGAATGGCCACTAAGCCACTAAGATAACA
CACAGGCCTCAGTCTGTCCCGAGATTCTCCGGTCTTTCAGATCCTGTTCAAATCTTCCTGTTCATCTTCCATTTCACAAAACCCAAATACCAAA
CCTAGAGTTAGTTTTCAGTTCACTCCGCCGATCATTCACGATGACTGTTGGAAAGGATGCTTATAACCCACGGCGGTTTCAAGCAAACGCCAAG
ACCAGGTCTAGGATATTAAAGCTGTTGGCGACGCCTTCCTCAATGCCGCTCGTTTTGATGGTTCTGCTGCAATAGTCTATGCTTCATAGGTCCA
TTTGGGGCCCTTCCCACGGCCATGCTGAGTGTCTGCATTTTGAAAACCTGACAAAGCTGGAAGTCAGTTCGAGAATATTGATCCTGGGTAGCTG
AGATGGTTCAGTGGGTAAAGGCGGTTACCGAGCAACCTGGCAACCTGCGTTTGATCCCTCGAACCCACATAAAGATGCAAGGAAAGAAACAGCT
CCATAAAGTCATCCTCTGACCTCTACACATTCACCGCGGCACGTGTGCGCCTGCACACACATCACATACGATAATAAAGAAAAAAATTTTTAAA
GATTTGACCTTTAGTGTACCAAAAGGAGAGAAAGCATTCATTCACTTTTAAACCCCGTGCATATTTACTCCAGTGGCTTTGAAGGGAATGGGGA
CAAACAAACAAACAAATAAATCAAGGTCTTTGTTTAACAGGTTGTGAACCATGGCTATGTGGATACAGGCTCAGCAGCTCCAGGGCGATGCCCT
TCACCAGATGCAGGCCTTGTACGGCCAGCATTTCCCCATCGAGGTGCGACATTATTTATCACAGTGGATCGAAAGCCAAGCCTGGTAGGTGAAC
AGCTTCTAGATCATCTTTTGGGAAGTACGAGGATATTGAACTTTGATGAGAAAAATGTTCTAACTGAGACTTTAAAAGTTGTGAGTAACCTAGG
CTTAGTGGTCCCCACCTGTAACCGTCATGATCCTAGCTGAGGCAGAAGGGTTGCAAGTTTGAGGCCAGCCTGAATTGCAGAGTGAGGCCCTGTC
CCTATCTTGGAAGAAGAAGAAGAGGAGGAGGAGGAGGAGGAAGAGGAGGAGGAGGAGCTCTGTGTGTCGGGCTGGGTGTGTAGCTCAGTGGTAA
AGTACAGGCATGAGGCTCTAGACTCAGCCCTCAGGACCTGTCTCCAAGAAAAGCTGTTTGAAAATCTTCATGTAATTCATAAAAAGGTATTCAG
CCTTCTTTTAGATGCAGCCAGACGAAATAATTCTTGCAGTTTTAATTTTTGTTGTTTTTTAAATTATGTATATATGTCCACGTCTGTGTATGGA
TGCGTACACATGAGGGCACCTATCTGCAGAGGCCAGAAAACAGCTGGAGCTCGCGTTTCAAGTGGTTGCGAACCACCTGGTGTGGCCGCTGGGA
TCTGAACTCAGGTCCTCGGGAAGAGCAGGAAGCTCTCTTAACCACACAGGCCTCTCTCCACCCTCTGAGCTTGCCTTGTTTTGGTTTGTTTGTT
TGTTTGTTTGTTTTTGAGACAAGGTCTCTCACTTTTTGAGACAGGCCCTGGCTGTCCTGGAACTCAAAAGTGCTGAGATTAAAGGCAAGAAAGA
ACCATGCCTGGCTTTTTTTTTTCCCCCCAAGGTAAAGTTTCACCGTGTAGTTATGAGTGGCCTGCATTCACTATATAGACCAGACTAGTCTTGG
ACTCAAGATCAGCCTTTGTCTCCCATGTGCTGAAATTTATATCTTATAAATCAGATATTTAACATATTGATTTAATAATCAAAACATTCATAGC
TCTCTGGTTTTTTGTTTTTCTGAGATAGGGTCTCATACTATATAGCTCTGGCTGTCCCGGAACTCACTAAATGGTTTTTTTTTTTTTTCTTTTT
AAAAACTAATAATGGTTACATTTCAGTGTTGACTTAGCCAACATTTTTATCTCTATTATGAATTATATACTTGAGCTTTAAAATTGTTAGATCG
GTATAACACAGTTCAGTAGCAATGCATCTTACCGTGCAGTGCTACGACAAAGCTTACAACACACGAGACAGCTGTACCACTGAGCGGCTGCCTC
CTCAGCCCTGCCGCATCCTCACCAGCCATCAGCGGTGCCCAGCAGCGTGCAGGGCAGGGAGCTTGCCCGTCGTTGGCCTCAGTCTCTCCGTGAG
TGTCCAAGTTCATCACAAGTTCATCAGACTAGCCAGGCTTTGACCAGGGTGGTGAGCTCCTCTTTGCAGAGCCAGCCTTGTTGCTGCTGGGAGA
AGGCTGTCCCTACCCACTGGAGATGTTGCCTTACAGTAATTGCTACAGCTTGTTAGCAAGGCTCCGTTCAGAAACAAGCCTGCCGAAGGAAGGC
AATTAAAAAAAATCCCAGCTTCATATAATTAAGAGGGGGAAAAACTGACTAAATATGAGAACATCAAACACACCTCAATTAGTCCAGTCCTTTT
CCTTTTCTTTCTGTTTTAATGATTTGGGAGATTTCGGAAAACATTAATAGGAAGTTTCTGCTCATTAAAAATCTCACTTAGAGGCTAGATCAGC
GGTAGAGCATTCGTCCAGCTAAGCAGACTGGCTCCATACCACAAAAGAGAGAACAAAAGCCAACTTTCGTTTATTCGAGTACAACAGCGCATTC
CTGTAATTCCACCTACTCAGGAAGCTCAGGCAGGGGAATTACTTAAGCCTAGGAACTCGAGACAGTCCTAGGCAAATGGGCTTTCAGCCAGCAG
ATCTCCTGAGTAGTCTACTTATTGAATAAGCTTTGGGAAATTGGGGTTGATATTAAGGAAAACGTGGGTTTTAGGTAGGGTTTTGTTTTTTCCC
CATAGAGAAATAGAGATATCACCGAAGAGTTTCCAGCTCTCCGTATTGAAGCTGTCATGAAGTCTCTGATTGTAGGCTATGGTTGAGTCCAAAT
TGAGAACCAGACTCCTGTGTTCTGTTCAAAGACTGGATCCTCTCAGCCGGGCAGTGGTGGCGCACGCCTTTAATCCCACCACTTGGGAGGCAGA
GGCAGGTGGATTTCTGAGTTCAAGGCCAGCCTGGTCTACAAAGTGAGTTCCAGGACAGCCAGGGTTATACAGAGAAACCCTGTCTTGAAAAAAC
AAACAAACAAAAAAAGACTAGATCCTTTCAACAGTGGGCTCCCAGTCAGAACAGAGGAGCAACGCTGTGCCTGCAGCCCGTCTAGCCTTCCACT
TCATACATATTTTCTTCAACCCATAGAGAATACACTTCTAAAAATTCGCTTTGTCCCTACCCTTTCCAGACACAGGTTGGGTGGTCTCTGCTAA
GGTCGTCTCAGGGCTTTGGTGCGCGTCTGCCTAGCTGTTTGCCACCAGCCACTTGCTAACTTCAGGGATTTGTGGGTGGCTGTCTTTTAGCTGC
CGCTGGCTCTCTTCCCTGGCTGTGGACCTGGTCAGCAGTAATTAGATATTATTTCAGCCTGACAGCTGTTTGGTTTGTCAGCGTGCTAAGTCAC
TTAAGTATTACCCAGCAGCAATTGTTGCCTAACTTAGTAGACAAAAATCTTTTAAGGACAAAAAGAAAGAAGATAAGGAGGAAGTGGACCCAAC
CCCTGTCACCAGCTGAACAGCTCAGGATGACACGAGGTCTGGGGACTGGCTTCTCGCCTTCACTGGAGGAGTCAGTGTTATTTCCAGGTCCCAT
ATCTGCAGTGATGACAGACTATTCTCCTGGGTGTGAATACTTGTAGGCATCTTCTGTCCTCATCCCTCCCTTCCAGGCTCCCTTAGGAAGGGCA
GTGACTGGGGGCCTGGAGCCAGTCAAGGTTGGAAAGAACATGCAGAACTATATACCCTGTCCCACATACCCTCGAGATACGAGTATAAACTGGA
AGTTAGAAGACCCCTTATTCCCTTTATTCCTAGGAGGGGGTGGGTTAATATCAGAGTCAGTCAGTAAAGGGAAGGGAGGAAGGAGTATTTAGAG
AAAGAAGTGTAAAGCTCAGCTAAAAGAATTGTAGAGTCAGGCATTGTAGACATTTAGCCAACTTATGTCGGGGTGTTTCTTAACCCGTGAATTA
TAGAAGAAATCACGTTCCAGACTCCCATGATGGCTCACACCTATAATCCCAGCACCCACAAAACAAATGTAGTATGTAAAGGCTGCATGTGTAC
AGGCTCCTATGCATGTATGTATACAGATTCTGTAATTTTTTTCCCCTAGGCACTCAATAGATCTTGATAATCCACAGGAGAACATTAAGGCCAC
CCAGCTCCTGGAGGGCCTGGTGCAGGAGCTGCAGAAGAAGGCGGAGCACCAGGTGGGGGAAGATGGGTTTTTGCTGAAGATCAAGCTGGGGCAC
TATGCCACACAGCTCCAGGTGGGTACAAACACTGGCCACACTTAGGCCCTGCCCTCTTGAGGACTTCTTCATTTAATTTTCTGTAGACACGTGT
TCTGATCTTTAACCCGGCATCGTAGAAATGTGTGTTTCACGCACATGGTTTTGATTGGCTTTGTGGTTGCGGCTGTTACCTTCTTTGAGACAAG
AGTCTCACCGTGGGGCTGGAGAGTTGGTTCTGTGGTTAAGAGCGCTTCCTGCTTGTCTTAGGGTTATTATTGCTGTGATGAAACACTATGACCA
AAAGTGAATTGAGGAGAACACCGTTTATCTGGCTTACACTTCCACATCACAGTTCGTCATTGGTGGAAGTCGGGACAGGAACCCAAACAAAGCA
GGAAGTTGGTGGCAGAAGCTCATACAGAGGCCATAGAGGAATGCTGCTTACTGGCTTGCTCCCCATTGCTTGCTCAGCCTGCTTTCTCATAGCT
CCCAGAACCACCAGCCCAGGCGCGGCCCCACACACAATGGGCTGCGCCCTCCCACATCAATCACTAATTAACAAAATGCCCTATAGGCTTTCCT
ACAACCCAAACCTATCATAAGAAGGCATTTTTCTCCACTGACGCGCCCTCTTCTGATGACTCTAATTTGTCTCAGGTTGATAGAAAACCAGCCA
GCGTGCTGCTATTGTAGAGCACCTGGGTTCAGCTCTCAGCACCCACATCAGGAAACTCACAGTAACCTGGGGCTTGGAGCCCTCTTCTGACCTC
AACAGGCATCCACCCACAAGTGACATACAGACACAGAAATAAAAATAGAAACTAATCTTTATTTAAAAAGAAATGAAAAGTTTCACTATTTAGG
TCAGATTGGCCTCAAACTGCCGATCCTCCTCCATCAATTCCACTCCACCCCCAGTATGGGCTGTAGGTAAGCACCACTCCCAGCCATGACTTTT
ACTCTCTTTTAACTTCTTATTTCAAAATAATTTTGAATTTGCAGAAAGTTGCAGCAGAGGTACAACAAGGCCCCCTTCATATGCTCAGGTTCAT
TTACATCATCATCTCTCTCACGCTCCTCTCTCCCTTTCCCGCCCCCACACCTTCATGTACACATGTACACATACACACATACACACTCTCACAC
ATACACACATACTCATACAACACACACACATAGTTACACAATACACACACATACAAACATACACATACAGATGCAGACACATACACACACATAC
CTTCACGTACACATGCACACACACACACACACACACACATACACACACACACACATTTCTAGTTGCGAGTAAGTTACATACATCTCTCCCCTGA
GTATCTCACCATGTATGTTCTAAGAACATGAACTTCCCTTACAGGGTCACAATACAGTTATTCAATTTAGTGTGTTTAACATTCACACAAACTT
TTACTTACTATTCTGTCTCTATTATCCATTTGTCAACTGACTCACAAGTGTCCCTCCAGTCCAGGGTCCAGTCCAAAGCCTCATATGATATCTA
GTTCACATATCTCTTTAATGTTTTATCTGGTTTGGTTTCAACTTTGTGACGTGTCTGTTTTGGAGATAGGGCCTCCATAGTGTTGTAGCCTATG
CTAGTCTTGAACGTTAAGCATTGAACATTAAGCAGTCATCTTGTCCCAGCAGTCCTGTCCACCTTTCCCTTTTCTTTAACCTCAAAACATTCAT
TTTTCGACCTTATTTTGGCTTTGATATTAACATTTTAAAATAATCACTTATTCCCTTACTTTTAATATTTTTACATTTTATTTTTAAATTTGTG
TGTGGCTATGTATGTCCACATGAGTGTAGTGCCCTCAGGGGTCAGAAGAGGGCATCACAGCCCCTCCAGTTGGAGTTAGAAATGGTTGTGAGCT
GCCTGAGGTCGGTGCTGGGAATTGAACTCGGGTCTTCTGTGAGAGCATTATGAGGTAGGGCCTCACCATGTTGTCTAAGCTGATCTCAAATTCT
AAATAACGGATGCATTGTCCTTCTGCAATGCCCCATCCAGAGAAAGAAGATGCCGGTCTTCCCTACTGTGACAGTAATTCTGATTCTAACTGAC
AGGAAATTTCCCAGTCCTGTGTCTTGTCTGCTGTGGCCTTTTAAAGACTCACCCGTTTCCAAGAATGTTGCTGTAGTGCTTTTGGCATTGAGGT
TCAGAAAGTAAAGCAGCCTTACCTTCTTAGTGCAACTGCATTTTCATAATGTCTTTCTCTCCATCCCGCCCACACCGGGGAACAGCACTGCCCA
TGACTCCATGGTTACCGCATCTCTACCATGAGTGCTCCCTGCCCTGGGTCCGGTTAACCCACAACAGAGTGGAGCAAAGTGATGTCTGCTGTTG
TCCTCTTTCAGAGCACGTACGACCGCTGCCCCATGCAGCTGGTTCGCTGTATCCGCCACATTCTGTACAACGAACACAGGCTCGTTCGCGAAGC
CAACAACGTGAGTGTGCCCATGGTTGTGGGAGAGGAGTTGGGGTGCCCTTCTTGAGCCGTTTCCCCCTGGATCCCACTTTGTTGAGGTGACCCA
ATATGCACTAGTGTCTCTTTAGGGAGCTGACCTTAGCTTTATAACTTAAGTGAGGCTACAACCACAAAGCAAGGTGAACATGTGCAAACTAACT
TAACCAAAGCAAATCAGTTTTGTGTTGAAGCATGCTCACAATCAGGGAAATTACTTAGTAGAAATTTCTTCACACTCTCTGGGCCAGCTGCTGA
AGGAGTGGATCATACCTAGTCACTTAAACAGCCAGATCCACAGGGACCTTGTGCATGCCTCCCCCATCACTTCCTCTGAGGGGCTACAGACACC
TAGGAGCCTTCGGGTGTGGCTTCATCCACGGTAGAAGCAAATGGGTAGAAGCAAATGATTTCAGCACCAGTCTGCCTTGAGATAGATACAGACC
ATGTTTTATTCCCAATGTCTCCCCAGCGAAATCACAGTCATGGTATTGGATCAATGAGTGAATGTGGAATCAGGGTTATCATCATCAGGAGGAA
GACCCAAGTGACCTCTGGGGCATGTGGAGTTCTAGTTTCTCAAAAGAATTGGGCACACACATCCTCTGTGGGACACTCATGTGGACGCCAGAGC
TGAAACAGAAGTAGGTTTTTACCGTCAAGAAAGCCTGTCGAGTCAACCGCCTCCACTCGCTTTCCATTCTACCCCGATTCTCACATGTGGCATT
TGTGATGTCTTCTGACCTCAAATTTAACCTTCGGGGTCTTAGCTTCACCTAGGGATTAAGACCGAAGGCTTCTTCCCACCCTCCCACACTGGAT
CTCCCCACCTGTCCCCAGGGCAGCTCTCCAGCTGGAAGTCTTGCTGACGCCATGTCCCAGAAGCACCTTCAGATCAACCAAACGTTTGAGGAGC
TGCGCCTGATCACACAGGACACGGAGAACGAGCTGAAGAAGCTGCAGCAGACCCAAGAGTACTTCATCATCCAGTACCAGGAGAGCCTGCGGAT
CCAAGGTGAGGCTGGCAGTGGAATCCTGGAAGCTCCGGGGGTGGGAGTAAGGTCTAGGAGCAAGCTCTGAGGAAGCAGAAGAGATCCTGGTTCC
ATTCTGAGCTGTGCCTTTGTCTGAGCAGAGTGGAACCATTCAGGCTGAAGAAGGAGCTGGGTAGCTTAGGGAGAGAACATGGCACCTGATGGGG
AGAGTGCAGGGTCTCACTCAGGCCTTCCTGTTCTCATCAACTTTGGGGTCGCTGTAGAACATGTTTCCTTTTTGACATTTAACGTGTTCATGGG
CTGGAGAGATGGCTCAGTAGTGGAAAGCACTGGCTGCTCCTCCAGAAGGCCTGAGTTCAATTCCCAGCAACCACATAGCAGCTCACAACCATCT
GTAACTGTAGTCCCATAGCGGCCAATCCCCTCTTACGCTGTGCATATATCCACACAAAATATGCATATATATAAGTAAATAAATCTTTGCCAGG
CATGGTGGCTCACATCTTTAATCCTAGCATTTGGAATACAGAGGAAGGTGACTCTATGAGTTTGAGAACAGCCTGGACTACAAAGAGAGTTCCA
GGACAGCCAGGGCTATTACTCAGGAACTCTATCTCAAAAACAAGCAAAAAGCATTCATAGCTTAATAAATAGCTTACAGAGGACATATTATAAT
TGTATATAGTAAACATTTTCTTTTTTTAAAGATTTATTTATTTATTATATGTAAGTACACTGTAGCTGTCTTCAGACACTCCAGAAGAGGGCAT
CAGATTTTGTTACGGATGGTTGTGAGCCACCATGTGGTTGCTGGGATTTGAACTCAGGACCTTCAGAAGAGCAGTCAGTGCTCTTGACCATCTT
GCCAGCCCCAAAGTAAACATTTTTAGAGTAATTGTTTGTTTGTTTTAGACAGGGTTTCTCTGTGTAGCCCTGGGTGTCCTGAGACTCACTTTGT
AGACCAGGCTGGCCTCGAACTCAGAAATCCGCCTGCCTCTGCCTCCTGAGTGCTGGTATCAGAGGCGTGCGCAACCACTGCCGGGCCTAGTGTA
GGCATTTTATGAATGCAGCCTTGCTGGGATGCGAAGTAGGAAGTACATCACATCTAGGAGTGTGGGTGTAGCTCAGAGGTAGAATGCTTTCCTA
ATGTGTGAGAGCCTGGCCTCCATCCCCAGCACTGCAGCATCTCTATTTGTGGTGCCTGCCATTGACTTGTATGCAGTTATTCAAGACTTTCCTG
TGACCCTGTATGCCTTCTGGACAGTGTTCCCTAGCCTTTGTTCTAAGGCACACAGAATTTGAGAATTCTTCCAGGAGTTGTGGAGCCTCCGCAG
CGCTCCCTCTTAACCCTACGCTTTCTTCTCTATGCGGGCGTGCCTGCCTCAGACAGACAGAGGGGGCTGGGGCCATGGCACTAGTGATTAAGAG
CACTGGCCTCTTTTGCATAGGAGTTGGATTTGGTTCCCAGAATCCACTTGGTGGCTCAAAACTGTCTGTAACTCCACTTCCGAGTAATCCAGGA
CCCTCTTCTAACCTCTGTGGGCACCAAGCATGCACGTAGTACACAGACATACACGTATGCAAAACACTCATACGCGTGCAATAAATCTAACACC
AATTTTAAAAATAAGACTCTCAGAAGAAACCTGTCCCCACACCCGCCTAAGGTCTCCTAAGGTCATGTGTGTAAAGCATAGCGCATTGCTATGT
TGACATTATTTACTGCCTCCCAGGCTTCCCAGATGATAAATCATTGAGAAGGAACTCTGTCTATTAGCTCTGTGCCCTGAGGCCAGCATAAATT
ACAGTTGCGGGCTTATCCTGGCTGTCTTAGTGTTGCTTTTGTCCATCCCAGCTTGTGTGTCCCTGGGGCAAGGACAAGGTCATTGTGACATGGT
TTTCAGAGGCCAGCCATGAGCTTCAATGAGACATGCCCGTATGCACAAATTCTTGCCAAGACCAGAATAAGGATGCTGGGCTACTCAAGGGCCA
CCGGAGGAAACAATAATGCCAGGACTCCGGTCTTTTTCTGGGCATGGAGCAAGGAACAAGAAGGTTTGCAGGCAGTGATAGTGGACAGGTAGTT
CAACCCACGACGTACTTGATGGAGCAGAGCCGATGAGGACTCTATGTGACTCTGTCTCCTGCTTCTGTCTCCCCGGTCTGTCCATTCGTATTGA
AAGGTACCAAAAGAAAACCGACGATGTAGCTCAGTTGGGGGAGTGCTTGCCTAGAATGCAAAAAAAAAAAACAAAACAAAAAAAAAAAAAAAAA
ACCTGGTTTTATCCCCAGAGCAGCCTCCTGAGCCGCTGTCAAGAGTCTCCCTTCACAGATGGAAGACTGAAGCCGGAAATACAGGGATTTACCC
AAGTTCGACCAGCTGATAAACCACAGGGCCACTTGCACACATAGGCTAACTTCAAACCTGTGGGGAGGGCAGGATGCCCTCTGTGTGGGGAGTG
TGGAGGCACACACTACTAAGCAGTTGCTTCCCCAACAAACACCTGCTCCCGGCCTAGGAGAGGCCAGGTCAGGAGAACCTTCATCCGCCTTCTG
AGAGAGCCCCTTCATCATGGGACACCCATTGCGAGCTGGAACTCTGACTTACTGTTGATGGGGTGTCTAGATGACCCTGAGAGTGGGTGATCCT
TCAGACTTCTTACTGGACACAAGAATTGATGGTGGTGGGTGTCCTGTCCCCTCCTCCAGCTCAGTTTGCCCAGCTGGGACAGCTGAACCCCCAG
GAGCGCATGAGCAGCGAGACGGCCCTCCAGCAGAAGCAAGTGTCCCTGGAGACCTGCCTGCAGCGAGAGGCACAGACACTGCAGCAGTACCGAG
TGGTTAGTGCGTGCCCTGCGACCCTCTGCCCCAGAGGGAGCTCTGTCACCTGAGATTCAGGGCACCAGGTGGGGGACAGAGCCTCGATGGGAGC
AGGGCCCCTCTCTCCTGGGGTCGACCGGGTGTGAGGGGGCCAGGCCCTGGGTTCTACCCCAGGCACTGAGCCATGCTCGCCGGGAGCCAGAGAG
CCGCTGTTCCCTCGCACAGGAGCTGGCTGAGAAGCACCAGAAGACCCTGCAGCTGCTGCGGAAGCAGCAGACCATCATCCTGGACGACGAGCTG
ATCCAGTGGAAGCGGAGACAGCAGCTGGCCGGGAACGGGGGTCCCCCCGAGGGCAGCCTCGACCTGCTGCAGTCCTGGTGAGGGTGAGAGGTGG
GCGGGGTGGGCAGCTTCGCCAACGATCCCAGCTGCCTCTTCTGTCCCTGCCGTGGCGTGAGCAGCACATGCCCACTGTCTGTGACAGCTCTCAC
TTTAGCAGTATCTTGGCTCTTGATGGCACTCGGCTCTTCAGACCTGTGCCACCTCCCACAAGGGACAGTGGTGCCTTGGGATGGGCAGGGCTAC
CCTTGAGCTGGTTGGTTCCAAGTTGGGGCCACCCTGACTGTCTTCTGCTTTTGAAAAGTGGGACTCTGCTCAGTGTGCCTGTGCAGGAGAGCGG
TCGCCAACGCCAGCACCTTTCTGGGGCGGCCCCTCCTTTGGTGGGAAGGAGGAAGAGTAGCATGCATCTTCCTCCCCTCGCCTGGGCTGGAACA
GCAGCCGCCATCCCCCTGAAGGCCCCCGGCTCCTTGCAGGTGTGAGAAGCTGGCCGAGATCATCTGGCAGAACCGGCAGCAAATCCGCAGGGCT
GAGCACCTGTGCCAGCAGCTGCCCATCCCATGCCCCGTGGAGGAGATGCTGGCTGAGGTCAACGCCACCATCANNNNNNMNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNCTCACTTTTTATGGAGTACTGGGCCTAGACCCCAGAGCCTTG
TGCATGCTGGGCAAGCATTCTACCAGCTGAACTATAACTATCCCCAGCCCATGATGAACATTTTCAGAATGTCTCCATGAGGAGGGGTCCTTAT
TGCATGCTGTTAGAAGAACTTAACTCTCAGAAGCGTGATAAAAGTGTCCTCGTTATTCGACACATCCAAGTCTGTCTTTCTCTAGTGTGAGACT
CTTGGCTCTTCAACTGTCATCTTTAGAGAACCGGGCTTTATTGCAAAGGCAGGGTTTTTATGAGATCTTCCCTGTGTGTGCTGCAAAAGCAGAG
TTTAGACTCCTAAAAAATAAAAAAAAAAATTAAAAAAAAAAAGGCCACAGTACACTGCTTTCAAAGTTTCAAAGCCTTATGGATTAGATTGCTT
CACCTCCATCCCTGTTGTCCACCAAGCTTTCTGTAACCTTGACGTGCCCTGCAATGTGTAACCAGTAGGGTATCTTGTCCAAATAATATGAAAT
TTTTTACATTCTCAGTGATGCTGATGACTAAGTTATCTTGAATGCTATTTAAAAATAAATACAGAAAGTTTGATAGAGTCGACCATCTCAGTAC
TTGGGCAGCCAGGACTACATAGAGAAACCCTGTCTCAGGAAGGAGGGGGGGCGAGGGAGAGAGAGAGAGAGGGAGAGAAAGAGAAAGGGAGAGA
GAGGTGTTATGCATGTTGGAATCCAGCTGTGATTCTGGGTCCTCCATTTCCTCCCTCTCGGAGTATCCAGACACCACTGTATGCTCGGATTTAT
TCAGGATAGGCTTGTATGCATTGTATGCATACACACTTTATTCGGGGTGAACCAGGGCTGTGTATATGAACCTAGGAGAGTGGAAAGGGGTTCT
TCTTAAAGTGAAATTACATTGGCTGAGGCTTCCGGCTACCTCATTTGCATGGAGAGGCATTCCTTGAATTTCACCATGGATTATGTGACCTCCC
CCAGAGTATGGTTTGGGCTCCCCAGATCAGAAGAAGAGGAAGCCCTGCTTAGAGAAGGTTGTTTTTTTGTTTTGTTTTGTTTTGTTTTGTTTTG
TTTTGTTTTGTTTTGTTTTTTTGGTTTTGGTTTTTTGAGACAGCGTTTCTCTGTGTAGCCCTGGCTGTCCTGGAACTCACTTTGTAGACCAGGC
TGGCCTCGAACTCAGAAATCCGCCTGCCTCTGCCTCCCGAGTGCTGGGATTAAAGGCGAGAGCCACCACGCCCGACTGGTTGTTCTTCATTTTA
CACCAAGCTCAGGGAAGCTGTCCGGACATGGTAGACTATATAACCTTAGTAGTGAGGTACAACAGACAGACAGACAGATGATAGGTAGTCCCCT
GAACAGTCATGGTAATGCACTCCTGTAATCTTAGCCCTTGGGAGGTAGAGTCAGAAGGATCAGAAGTTCATGTTTATCCTGGCTATATATTAAT
TTCAAGTTCAAGGCCAGATTGGCCTGCATCAGACCTATCCCAAACTAAGAAAGGGGCTTGGGGGATGGCTTAGTAAAGTGCTTGCCATGCAAAT
ATGGAGACATGAGTTTTTATATTTTATTTTATACATGTGAGAGTTTTGCCTACATGTATATGTGTGTAGCATGTTGTGCCTGTGGCCCATGAGG
CCAGAAGAGAGCATCAGATCCCCTGGAACTGGGGTTACAGATGATTGTGAGCTGCCATGTGGGTGCTGCAAAAACAGCCAGTGCTCTTCATCAC
TTGAGCTATCTCTCCAGCCCCAATAATAAATTTTAAGAAGTGGAGAACTCATGAAAAAACACACCTGAGGTTGACCTCTGACCTCCACCTATGT
GCACATATCTGCTTCACCCTCACACACCCCCACAGGGACGGCAGCAGGAAGGAAGCAGGAAGGTGAAGAGAGAGGAAGTCCCCTCTCCTGGCCA
TCTTGGTCACTGCCCTGTGTGGTGCGAGCACCAGACCACATCCCTCTGGCCCTAACTACCCTCGTACCTACTGACCAGCCAGCTGAATAGAATG
AGAGGCAGTGTATTACAAGTTCATATTCTAACTGTTTCTCAATCAAGTTGCCAACTAGAAACTTTCCACTCCTCCATGCCAAGTTGCTTCACTG
TGAGCTAGAGTTCTTGGCAGGTGTCAAGGACAAGCAAGTATTATACTTGGGGCTGCACATGTGTGCCCTCCCTCTGCTCACTTAGAAAGCCAGG
CTTGTCTGTCTGACTGCTCTCTCTCTCTCTCTCTCTCTCTCTCACTCCCTTCTTCCCTCNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNCAGCAAGCAGCAACT
GAGGGGGGGTGGCGGGGCGGGGTGGGGGGAGTCCTGTTTACTTAGCAGTGGATTCCCTGAAGAGCAGTAAAGTAGAAGTGAAGGGGGGAGGCAT
GGCAGGGAGGGGGGATCCATCTCTCCTGAGGGGTCAGAGGAGGTTTCTGAGGAAGTGACACGTGAATAGACTTGAACAAAGTAAGGAAACTGGC
TTTGCAGAGACCCAGAGGACAAGCATCCTAGGCTGAGACACAAAGCCCACCTCGGGTGGCTGAAAAGCTCCCCAGAGCCCCTAGGCTGCCTCCA
GCCAAGCAGGAAGTTGGTGATGGGAACTGAGGCCAGGGCACCTCAGTAATGGAGGTCGGGGTGAAAGGTCAAAGCGTGCATAATCCTAAGTGCC
CTGATTTCACACACACACACACACACACACACACACACACACACACACACACACACAGTTGCAGGTCAGTAATGCATTCATGTAGCACTCTCTT
CAGGATTGCACAGGATTCCATTACCTAGTGACTTTCGAAGATCACGGTTAGGCAATTGAGATTCTTAGTATTTAATTTACTTCATTTTCACTAA
TGACTGATATTGAGATAACCTAGTACAAGTACCTTGTTCAACTATTCCTTCAGATAAATTCCTAAGCAGAATTAGGTCAAAGGGCATAAGCTTT
ATAAGCTTTTTTTTTTTTGACGCATCCAAAACTGCATTCAAAAACCCAAATTCACATTTCCCACACCTCTCTCTTCCACATCTGCTTCTCTGTC
TTTTTGCCAACCACAGAATAGCCTCCATTCATGTCTGCCAGTCTGGTTGGCATGAAAATCGTACATTATAAGTCCTGTGTGTATTTATCTGATT
CTAGGCAACCTTTGCATATGATGATTAATAATTCCAGATTAATTAGCATGGTAGCAGAAGCTAGCATTTGCTGGATGGCTCTGAGTTTCCTAGA
GTATCCGTCTTAGTCACTGTTCTACCACTGTGGAGAGACACCGTGACCAAGACAGCTCTTACAAAGGAAAACATTTCATTGAGAGCTGGCTTAT
AGTGTGACTGGAACTCCTCTTTTACAGGAAACTGAGGCATAGGGAAATCAAATAATTTGCCAAAGATCCCAGGACTGGGAAGGGGCCAAGCCAG
GGTTTCAGCTTGGCTTAGCTCCTGAACCTAGATTCTTCATCCACCAATGGCCAGCTCTGGCCATTGATAATTTTTTTCTGTGAAAAACATGTTT
GTATTTCTTCCTTGGTTTTTTGAAGTTATATTTTTATATAGAAAATATACTCATCTGAGGGTGAGATGCTCAGTCTACAGAGTACTTGCTGCGA
GTTTAATCCCTAGAGCCCATACTTTTTAAAAAGCCAGGTGATGTGGCACTTGTAATACTAGTGTATGTGTTCCTCCACATGCCTGGGCACACAC
ATGCATGTACACACATGCACATAAATATGCCGCACAGAAACATGTGTGTATATACACACACACAAGAAAAATAGTTAATCCCCTATTGATTTAT
CTATACAGAAAATCTTTTCTCTGTATTAAGTTTTATTTTCTCAAGAACAAAGACTTGTGATTTCCATAGAAACCCAATCTGTCTGTTTTTAGTG
TGTGTGTGTGTGTGTGTGTGTGTTACACACATATACCATGGTGTGCATGTGGGGAGTAGGAAGACAGTCACTGGGAGTCGGCAGTCTTCTTCCA
CTGTGTGAGTCCCAGGGGTGGGACTTGTGTGATCAAGCTTTGTGACAAGGACCTTTACCCACTGAGCTGTCTTGCCAGCTCCAACTGTGAAGTT
TTCAAAGCTTTTATTTTTCAATTATGCCTGCAAGCACAGTCATAGTCTCAGTTTGGGAGGCTGAGGCAGCAAGATCCAAAATGCAACACCTGCC
TGGGGTAGAGAGTGAGTTCAAAGCCAGCATGTGCAACTTAGCAAGACACTGTCTGAAAAGAGGTGGGGGGATCTTAGGGGTAGAGCTTTTGCCT
AGTATATACGATGCCCTAGTATATACGATGCCCTAGGTTCTTCAGTCCCCAATACCAGACAAAAAGTCAGACATAGTCATAAATTCATAAGGTA
GAGACAGGCAGATCTCTGTGAACATGAAGGCAGCCTATCCTACATAGTGAGATCCTGCTTCAAAAGAAAAAATTTTTTTTCACAAATATGCACC
ACTAGAAGTTAACTACACGCGCATGGAAGTGTCTGCCTTCTCTGTCCGCACTTTCTAAACCGTCCCACCTGACCTGGAAGGTGGCCCAATTTGT
ACTTTCAGTGCCTCACATCGGCAGCAAGCCTCCCAGAAGACAGAAATGTTACCTGCTGTGTCGGTCATGTTCTCAGTTGAGATTAAGAGGCTGT
GTCCTAACCCTAGAAAAGATAAGTTCTTGAGGGAGGTGTCTCTGAGGTCCCTGGTTTTCACTGTTCTTCTCCCCGGCAGCGAAAGCAGCTGACG
GATACGTGAAGCCACA
MOUSE SEQUENCE - mRNA
AAGGTCCTGCAACGGGGAGGGAGCGAGCGGGCGTGCGAGAGAGCGTGCGGGACCGGAGAGACCCTGGCTCTGGTGGGGCAGAACGAGGTTGTGA
ACCATGGCTATGTGGATACAGGCTCAGCAGCTCCAGGGCGATGCCCTTCACCAGATGCAGGCCTTGTACGGCCAGCATTTCCCCATCGAGGTGC
GACATTATTTATCACAGTGGATCGAAAGCCAAGCCTGGGACTCAATAGATCTTGATAATCCACAGGAGAACATTAAGGCCACCCAGCTCCTGGA
GGGCCTGGTGCAGGAGCTGCAGAAGAAGGCGGAGCACCAGGTGGGGGAAGATGGGTTTTTGCTGAAGATCAAGCTGGGGCACTATGCCACACAG
CTCCAGAGCACGTACGACCGCTGCCCCATGGAGCTGGTTCGCTGTATCCGGCACATTCTGTACAACGAACAGAGGCTGGTTCGCGAAGCCAACA
ACGGCAGCTCTCCAGCTGGAAGTCTTGCTGACGCCATGTCCCAGAAGCACCTTCAGATCAACCAAACGTTTGAGGAGCTGCGCCTGATCACACA
GGACACGGAGAACGAGCTCAAGAAGCTGCAGCAGACCCAAGAGTACTTCATCATCCAGTACCAGGAGAGCCTGCGGATCCAAGCTCAGTTTGCC
CAGCTGGGACAGCTGAACCCCCAGGAGCGCATGAGCAGGGAGACGGCCCTCCAGCAGAAGCAAGTGTCCCTGGAGACCTGGCTGCAGCGAGAGG
CACAGACACTGCAGCAGTACCCAGTGGAGCTGGCTGAGAAGCACCAGAAGACCCTGCAGCTGCTGCGGAAGCAGCAGACCATCATCCTGGACGA
CGAGCTCATCCAGTGGAAGCGGAGACAGCAGCTGCCCCGCAACCGGCGTCCCCCCGAGGGCAGCCTGGACGTGCTGCAGTCCTGGTCTGAGAAG
CTGGCCGAGATCATCTGGCAGAATCGGCAGCAGATCCGCAGGGCTGAGCACCTGTGCCAGCAGCTGCCCATCCCAGGCCCCGTGGAGGAGATGC
TGGCTGAGGTCAACGCCACCATCACGGACATCATCTCAGCCCTGGTCACCAGCACGTTCATCATCGAGAAGCAGCCTCCTCAGGTCCTGAAGAC
CCAGACCAAGTTTGCCCCCACTGTGCGCCTGCTGGTGGGGGGGAAGCTGAATCTCCACATGAACCCCCCCCAGGTGAAGGCGACCATCATCAGC
GAGCAGCAGGCCAAGTCCCTGCTCAAGAATGAGAACACCCGCAATGATTACAGCGGCGAGATCCTGAACAACTGTTGCGTCATGGAGTACCACC
AGGCCACTGGCACGCTCAGCGCCCACTTCAGAAACATGTCCCTGAAACGAATCAAGAGATCTGACCGCCGTGGTGCAGAGTCAGTAACGGAAGA
GAAGTTCACGATCCTGTTTGACTCACAGTTCAGCGTCGGTGGAAACGAGCTGCTCTTTCAAGTCAACACCTTGTCGCTCCCGGTGGTGGTGATT
GTTCACGGCAGCCAGGACAACAATGCCACAGCCACTGTCCTCTGGGACAACGCCTTTGCAGAGCCTGGCAGGGTGCCATTTGCCGTGCCTGACA
AGGTGCTGTGGCCGCAGCTGTGTGAAGCGCTCAACATGAAATTCAAGGCTGAAGTACAGAGCAACCGGGGCTTGACCAAGGAGAACCTCGTGTT
CCTGGCACAGAAACTGTTCAACATCAGCAGCAACCACCTCGACGACTACAACAGCATGTCCGTGTCCTGGTCCCAGTTCAACCGCGACAATTTG
CCAGGACCGAATTACACTTTCTGGCAGTGGTTTGATCGCGTGATGGAAGTATTGAAAAAACATCTCAAGCCTCACTGGAATGATGGGGCTATCC
TGGGTTTCGTGAACAAGCAACAGGCCCACGACCTGCTCATCAACAAGCCGCACGGGACCTTCCTGCTGCGCTTCAGCGACTCGGAAATCGGGGG
CATCACCATTCCTTGGAAGTTTGACTCTCAGGACAGAATGTTTTGGAATCTGATGCCTTTTACCACTAGAGACTTCTCTATCCGGTCCCTCGCT
GACCGCCTGGGGGACCTGAATTACCTCATATATGTGTTTCCTGATCGGCCAAAGGATGAAGTATATTCTAAGTACTACACACCGGTCCCCTGTG
AGCCCGCAACTGCGAAAGCAGCTGACGGATACGTGAAGCCACAGATCAAGCAGGTGGTCCCCGAGTTTGCAAATGCATCCACAGATGCTGGGAG
TGGCGCCACCTACATGGATCAGGCTCCTTCCCCAGTCGTGTGCCCTCAGGCTCACTACAACATGTACCCACCCAACCCGGACTCCGTCCTTGAT
ACCGATGGGGACTTCGATCTGGAAGACACGATGGACGTGGCGCGGCGCGTGGAAGAGCTCTTAGGCCGGCCCATGGACAGTCAGTGGATCCCTC
ACGCACAGTCATGACCAGACCTCACCACCTGCAGCTTCATCGCCCTCGTGGAGGAACTTCCTGTGGATGTTTTAATTCCATGAATCGCTTCTCT
TTGGAAACAATACTCGTAATGTGAAGTGTTAATACT
MOUSE SEQUENCE - CODING
ATGGCTATGTGGATACAGGCTCAGCAGCTCCAGGGCGATGCCCTTCACCAGATGCAGGCCTTGTACGGCCAGCATTTCCCCATCGAGGTCCGAC
ATTATTTATCACAGTGGATCGAAAGCCAAGCCTGGGACTCAATAGATCTTGATAATCCACAGGAGAACATTAAGGCCACCCAGCTCCTGGAGGG
CCTGGTGCAGGAGCTGCAGAAGAAGGCGGAGCACCAGGTGGGGGAAGATGGGTTTTTGCTGAAGATCAAGCTGGGGCACTATGCCACACAGCTC
CAGAGCACGTACGACCGCTGCCCCATGGAGCTGGTTCGCTGTATCCGGCACATTCTGTACAACGAACAGAGGCTGGTTCGCGAAGCCAACAACG
GCAGCTCTCCAGCTGGAAGTCTTGCTGACGCCATGTCCCAGAAGCACCTTCAGATCAACCAAACGTTTGAGGAGCTGCGCCTGATCACACAGGA
CACGGAGAACGAGCTGAAGAAGCTGCAGCAGACCCAAGAGTACTTCATCATCCAGTACCAGGAGAGCCTGCGGATCCAAGCTCAGTTTGCCCAG
CTGGGACAGCTGAACCCCCAGGAGCGCATGAGCAGGGAGACGGCCCTCCAGCAGAAGCAAGTGTCCCTGGAGACCTGGCTGCAGCGAGAGGCAC
AGACACTGCAGCAGTACCGAGTGGAGCTGGCTGAGAAGCACCAGAAGACCCTGCAGCTGCTGCGGAAGCAGCAGACCATCATCCTGGACGACGA
GCTGATCCAGTGGAAGCGGAGACAGCAGCTGGCCGGGAACGGGGGTCCCCCCGAGGGCAGCCTGGACGTGCTGCAGTCCTGGTGTGAGAAGCTG
GCCGAGATCATCTGGCAGAATCGGCAGCAGATCCGCAGGGCTGAGCACCTGTGCCAGCAGCTGCCCATCCCAGGCCCCGTGGAGGAGATGCTGG
CTGAGGTCAACGCCACCATCACGGACATCATCTCAGCCCTGGTCACCAGCACGTTCATCATCGAGAAGCAGCCTCCTCAGGTCCTGAAGACCCA
GACCAAGTTTGCGGCCACTGTGCGCCTGCTGGTGGGGGGGAAGCTGAATGTGCACATGAACCCCCCGCAGGTGAAGGCGACCATCATCAGCGAG
CAGCAGGCCAAGTCCCTGCTCAAGAATGAGAACACCCGCAATGATTACAGCGGCGAGATCCTGAACAACTGTTGCGTCATGGAGTACCACCAGG
CCACTGGCACGCTCAGCGCCCACTTCAGAAACATGTCCCTGAAACGAATCAAGAGATCTGACCGCCGTGGTGCAGAGTCAGTAACGGAAGAGAA
GTTCACGATCCTGTTTGACTCACAGTTCAGCGTCGGTGGAAACGAGCTGGTCTTTCAAGTCAAGACCTTGTCGCTCCCGGTGGTGGTGATTGTT
CACGGCAGCCAGGACAACAATGCCACAGCCACTGTCCTCTGGGACAACGCCTTTGCAGAGCCTGGCAGGGTGCCATTTGCCGTGCCTGACAAGG
TGCTGTGGCCGCAGCTGTGTGAAGCGCTCAACATGAAATTCAAGGCTGAAGTACAGAGCAACCGGGGCTTGACCAAGGAGAACCTCGTGTTCCT
GGCACAGAAACTGTTCAACATCAGCAGCAACCACCTCGAGGACTACAACAGCATGTCCGTGTCCTGGTCCCAGTTCAACCGGGAGAATTTGCCA
GGACGGAATTACACTTTCTGGCAGTGGTTTGATGGCGTGATGGAAGTATTGAAAAAACATCTCAAGCCTCACTGGAATGATGGGGCTATCCTGG
GTTTCGTGAACAAGCAACAGGCCCACGACCTGCTCATCAACAAGCCGGACGGGACCTTCCTGCTGCGCTTCAGCGACTCGGAAATCGGGGGCAT
CACCATTGCTTGGAAGTTTGACTCTCAGGAGAGAATGTTTTGGAATCTGATGCCTTTTACCACTAGAGACTTCTCTATCCGGTCCCTCGCTGAC
CGCCTGGGGGACCTGAATTACCTCATATATGTGTTTCCTGATCGGCCAAAGGATGAAGTATATTCTAAGTACTACACACCGGTCCCCTGTGAGC
CCGCAACTGCGAAAGCAGCTGACGGATACGTGAAGCCACAGATCAAGCAGGTGGTCCCCGAGTTTGCAAATGCATCCACAGATGCTGGGAGTGG
CGCCACCTACATGGATCAGGCTCCTTCCCCAGTCGTGTGCCCTCAGGCTCACTACAACATGTACCCACCCAACCCGGACTCCGTCCTTGATACC
GATGGGGACTTCGATCTGGAAGACACGATGGACGTGGCGCGGCGCGTGGAAGAGCTCTTAGGCCGGCCCATGGACAGTCAGTGGATCCCTCACG
CACAGTCATGA
HUMAN SEQUENCE - GENOMIC
AGCCCTCCCGTGCCTCCCACCCTGACCCTGTGCCCAGTGGCTGTGCCCTTGGTTGACCATCCCTCATTTGGTCTCTGACTGTAAGGCCCCGAAT
ATCTGTGTCTTTCATTGCACATTTCCCCTTCTGGGTTTTTTTTTTTTTTTTTTTTTTGAGATGGAGTTTCGCTCTCGTTGCCCAGACTGGAGTG
CAATGGCGCGATCTTGGCTCACAGCAACCTCCACCTCCAAGGTTCAAGCGATTCTCCTACCTCAGCCTCCCGAGTAGCTGGGATTACAGGCATG
TGCCACCACTCCTGGCTAATTTTGTATTTCTAATTTTGTATTTTTAGTAGAGGCGGTGTTTCTCCATGTTGGTCAGGCTGGTCTCGAACTCCCG
ACCTCAGGTGATCCACCCGCCTCGGCCTCCCAAAGTGCTGGAATTACAGGCATCAGCTACCATGCCCGGCCCCCACTCTGGCTTTTATTCTCTC
TGTGTCTCTCCCTTGTCTCCTTAGGTCTCCATCTGGTTCTCTGATTCTGTCTGGGTTTCTCTCCATTTCTTCAGGCCTGTTCTCCCTTTGAGAT
GCAGTGGGGAGGAGGAGGAGCTATGGAACTTTCCCAGGATGGGCAAAGGGCACCCAAGGGAGTAGAGGGAAGGGTGTCAGAGTTGGGTTGGGAA
GAGGTGACTGTAGTCGTGGCTCCAGATAACACAACTACCCAGAAATGTCAAATTTGGGGTTTCTTTTTGCTTTCAGTCCCCTTGTTTGTCCCCT
TGTGTGGGTGATTTCCCACCACAAAGTGCCCTGGTGATTTTGGGGAACCAGGCTGTGGAGGAGAAAACTGAGGCCACGGGATGGGGGTTCAGGA
AGATCAGAAACTGTCAAGGGTCTGACTGAGCTGCGGTCCGAGGCTGGCCTCCTTGCCCCTAACTGCTCTTCACCCCCAAATCCAAGTCACAGTA
TTAGATGGGGAGTGAGGAAGGATTTTAGAAATCTTACAGAAATATATTCCCCAGGCTAGGTGCAGTGGCTCACGCCTGTAATCCCAGCACTCTG
GGAGGCTGAGGTGGGAGGATCACTTGAGGCCAGGAGTTTGAGACCAGTCTGACCAACATGGTGAAACCTCGTGTCTACTAAAAATACAAAACAA
TTAGCTGGGCGTGGTGTCACGCACCTGTAATCCCAGCTACTCGGGAGGCTGAGGCACAAGAATCTCTTGAACACAGGAGGCGGAGGTTGCAGTG
AGCCGAGATCATACCACTGCACTCCAGCCTGGGCAACAGAGTCAGACTCTGTCTCAAAAAAAAGAAAAGAAAAAAAAAAAAAGCAATATATTCT
CCAAAAGAAGACCTCCTATATTTTATTAGGAATAATAATAGTCTGCAGCCTAGGCTTCAGTTGGCACACTATATTCGTATCCTGAACTACATCA
AGGCACATTTGTGTACAGACATGGTTGTGCTCTTCAACTCAACGGATCCTTTCAATAACCTTGAAGGCAGTCAGGGCCAGAATCACACCCTTTG
ATTAGTCCATGAAAGAAACCTGCCCAAAGCTGTCTGGCCAGTAAGTGCACAGATGGCACCAGGATCCAAGTTGCCGGGCCTTGTGGGCAGGGTC
TCAGTATAACCTTTCTAAGCTTCCCCAAGCCCAGCACACACCGGCGGAGTGAAGAGGCCTCCCTGGCCTGGGCTAGCCCACCATTTTTCTGCGG
TGCGGAGCATGGCATCTCCATTCTCGCCCAGCCTACACCCCTGCTCTATTGAATGGGACAGTGTGCTCTGTGCACACTTACATGAAGGTGGGTT
TGAGTTTATGGATTAAGAGAATTATCCCAAAGGGCCGGGCATGGTGGCTCACGCCTGTAGTCCCAGCACTTTGGGAGGCTGAGGCGGGTGCATC
ACCTGAGGTCAGGAGTTCGAGATCAGCCTGGCCAATATGGTGAAACCCCGTCTCTACTAAAAATCCAAAAATTAGCTGGGCGTGGTGGTGCACA
CCTGTAGTCCCAGCTACTCAGTACGCTGAGGCAGGATAATCACTTGAACCAGGGAGGTGGAGGTTGCAGTTAGCTGAGATCACGCCGTTGCACT
CCAGCCTGGGCAACAAAAGCGAAACTCCGTCACAAAAAGAAAAAAAAAAAAAGAGGGAGAGAGAAAGAATTATCCCAGGTCCCCTTTCTGCCTT
GCCCTTGACGATCAGGTCTGCCCATGCCCCTACCCTGGGGAGAACAGATTGAGGTCACCTTGACTTGACCTTCCTTGGGGGGTGGAGGGAGGTG
GTACAGAGATGCCTTTGTTTCCTGTGCTCCAAGTCCCTTCTTCAAGGAGAGTTCCTGTTACATGGGAGCCAGAGGGGAGGTGGGGTAGGGGTGA
AGGGCCTTCCCAAGAAAGCTGGAGGCTTTCTAGGAAGTATCTTTTCACCCTGCGGCAAAGGGGAGAAGCCCTGGAGCACTTAAACATCCTGTTT
CACCCCCACCTTGCCTCTGCTTGGACAGCCTACCTGCACTCCAGCCCCCACCGGAGAAGGCTTCTAGAAGTGGGGCCATGGGCCCATGCTCAGA
GTATACCCCACCTCTGAGGCCAGGCCCTGGTACCTCCCTATAGAAATGCCACCCCCATTCTTGTCCAAGAAGACAGATCAGGACCTAGGGAGGG
TGTGTGTTTATATGCACAGGGTGAGGACTTCTGTGCAAATCTGGGGGAAGGGAAGGAGCTGCCGGAGCTTGGGTGAAGCCCGTTCCTGCCGCTG
TCCCCTATCCACCACTCTTCAGGGAAGTCACTTTACAGTGAAGTCACCAACAGAACACAAGGCCATCCTGCCTGAGGCATAGTGACTGTCCTCT
GTTTCTGGGAATAGCATGAGGGGAAGGGTGTGGGGCTGGACAGGGTGGGTAGGAGAGGAGTCCTGCTTCCTCTGCCCAGCTGCCCAGCCTGCCT
GCCCTGGGGGAGAGGCTTGTAGGCCGGTTTCTTTCTGGACGAAGAGAAGAGGATGCAGAGTCTGTGCCAGGTTCACATTTCCCAGCCAGTCAGG
TGGGCAGGAGGAGCCCACCCTTTGCAGCCCCCTCATTGCAGGGCCAGGCCCAGGAGCCTGGAGAGAGGAGAGAGGGCACACAGAGCCAGGCAGC
TGAGTGGAATTGGCACTGTCCTGGGCAGCCCTGGGTCAAGCCTGGCTTCTCATCCTCATCCATGATGACTATCAGGCCGCTCTTCCCGCTGAGC
TAGTTTCTCCTCCTTCCAGCCCAACAAGAAATCTTTTGTGCCTTGGCCAGCCCAGTACCCAACCTCAAGCCCTCTGGGGGTTGTGTGCGAGGGG
GCTGAGCATCTTCCACGACACCTCCCAGTACTCTGCCACTTTCCTTTGTTATGACTGGAGGGGGTTCCTTTCTGTAGAATCTCTTTCTCATTGT
TTCTTCGTTTCTTAAGTCTCAGATCTACCCACATCTTCCTTGCTCCAAGCCTCAAAAAAGTGCTTCGGCCAGGCTTTTCCCCTTACGCTGGCCT
CTCAACTCCAAGCACTAGCCCCTTTCATTCCATCTACCTGGAATGTCATTCCTCCCTTGGCCACCTGGTGAAATTGGCCTCATCTTTCCTAGGG
TTAGTGTGGCCTAGAGGAAAACACAAAGGCTTTGGAGTCATAAGGTCCTGGTGCAGACCCTGGCTCTGCTGTAGGATCTTGAGTGAGTGAGTCT
GCCTCTTGGATCTAAGCATCCTCACTTGTAAAGAAATAGCAAGACCCACTGCTGGGATTTCTGTTAAGACCCAGTCACAGGCCCGGTGCGGTGG
CTCAAGCCTGTAATCCTAGCACTTTGGGAGGCTGAGGCAGGTGGATCACCTGAGCTCAGGAGTTTGAGGCCAGCCTGACCAAGCCAGCACAGAC
TCAGCATGGTGAAACCTTGTCTCTATTAAAAATAAAAAAATTAGCCAGCTGTGGTGGCAGATGCCTGTAATCCCAGCTATTTGGGAGGCTGAGG
CAGGAGAATCACTTCAACCTGGGAGGCAGAGGTGGCAGTGAGATGAGATCACACCACTGCACTCCAGCCTGCGTGACAGAGCGAGACTCTGTCT
AAAAAAAAAAGACTCAGTCACAGTGGCTGGTGCATTGTAGGTGCTCAGAGGCCTTGGCCAGTAGAGCTGGGTGTTTTTTCCTCTGCCCTTCAAT
CCCATCTGTATTACAGGCCCCTTCTTATGGCCCTAACACATTCCCTAAAGGGCTATGTCCCACTGCTTGTCCACAATAGAGTCTTCATAAATAT
CTGTTGGATTAATGAAGCCCCCTAAACCAGCACAGAACTTCCGGAGCTTTGACGGTTCAGGGCAGCCCCCTGTTCACTGACCACCCCCACCATG
GTTCCCAGAAGACTCTCCCCCTGCTGCCCTGGCCTCTGCCTGCTCTGGTCCCACTGAGCTGCTTTTCTACACACAGTGGCAGCAGCAGCCTCAT
CTTTGCCTGGTGCTGGGCTGAATTTTGAGTTCTTAACCTGCCTGGCAAGCCAGCAAGATACTGGCTGCAGTGGCACTCTGCTCCCTCCTCTGCG
TCCGCCCCACCCTCCCTCGATAATCCTCCATCCCCCAGGACTCTGCGTCGAGAGAGCCCAGTGACCCTGTCCCAAGAGGCGGGGTCGGGGGATC
CTTGGCCTTTCCAGAGAGCCCACCCTTTAGCCTTTAGCTCTCCCTGGAAGCCACCCCAGATTGGTGAGGCACACAGGAGCAAGACTGAGAGCAG
GTGTGTGAGACTCTCCAGGTGGCGAGTCCTCCATGCACCTGCACATCCATCGCTCCCGTCCTCTCCCCTCCCCTCCATGTGACAAAGCACCAGC
TCTCTCCTGGCCAGCACACTGGCTCGGATTAGGCTGAGGCAAAGAAAGAGAGAAAATGCTTTTTTATCAAGTGCCAGGCACAGTGCTTAGTTGC
CTTCACAAATGCTCTCCTTTTTCCTTTAAAAAAACTGTCTATTTTACTAATGTTTTAATAGGGAGCATCTGCACATGATATATGTATTTTGTAA
CAAAAGATTCAAAAGGGTATGTGCTGAAAAGTTAGTCTCTGCCCTTTCCTGTCCCCCAGGGCCCTTCTGGAGGGGCAACAACAGTTGCTGGGGT
CTTTTGTGCATTTAAATGCACGGAAGGATTCTGTGGATTTGAAACATATATTACACTCTACCCCCTAACTATGATTTACACGTGATGGGAAACT
GAGATCCTCAGAGACATTAAGTACCTAGACCAAAGTCACACAGCTAGTTTTTTTTTGTTTTTGTTTTTGTTTTTTTTTTTTGAGACGGAATCTC
GCTCTGTCGCCCAGGCTGGAGTGCAGTGGCGCGATCTCTGCTCACTGCAACCTCAGCCTCCCGGGTTCACGCCATTCTCCTCGCTCAGCCTCCG
GAGTAGCTGGGACTACAGGCGCCCGCCACCACGCCCGGCTAATTTTTGGTATTTTTACTAGAGTCAGGGTTTCACCGTGTTAGCCAGGATGGTC
TCGATCGCCTGACCTCCTGACCCACCCGCCTCGGCCTCCCAAAGTGCTGGGATTACAGGTGTGAGCCACCGCGCCCGGCCAACAGCTAGTAAGT
TAGCAGAGCTGGGATTTAGAACCATATCTAACTTTTCACCACCAATGCTCTGGCAGAAGTAAGCAGGGGCACTGACAGAGCCCTCCGAAACTGC
AGAGCCCATTCCAAACCGAGGCCACGCCATTCTGAGGCCTTGGAGATCTGGCATCCCCGCCTTCTTTTCTGTTGAGCATCCTATACCTTCTCCA
CATGGCTCCAGGGTCCTGTACTGACCTATCCCCCTATGGAAGGATTCATCCCCACAGGGAAAAAGGGACATTCCCACAGTGCCCTTTCTTAGCA
AATGTGTGGCCGGTGGTTGCCCAGGGTCAGCCTGACTCCTGGGATGCAGGAGGGAAGCCATAATAACGAAGGCCTCTGGTTTCTGGCCAGAGGG
CATCATCGTTGTGGCTTCAGTGACGTTCCCAGAAAATGCCCTCATGATCTCTGCGGCAGCAGTGAGCGAGCATGTGAAGCAGGGAAGCTGCCCC
CTCTGAAAGCCATGTGTTCATCTATGAGAGAGAGAGGGGAGGAGGCTGTAGGATGGGAGCAGGGGCATGCATGGACAAGGGCTGTGGACTAGAC
CCCCAGGCTCTTCCCCCAAGCCTCTCTCCTGGGTCAGGCTGGTGCACTAAGGGATGAGACCCTCCAGTGGACATTGCTGCATCTCCAGGTACCT
TCTTGGGTCATTCCTGATGGGTTGGACTGTGGGGTCTTTCAGGATCCTAGCTTCCTGTTCAGTTTCTTTCTTTCTGTCATGTCTTAAGTCTGGT
GTCCCCTCAATTTAAGGGAATATGTCCCCGAGGCCTAGAAGAAGAGGGTCCCACCCCATCAGAACTGTCAAGCAGAGAAGCAGGTGTCAGGCCT
TCCCATCCCAGGATGACAACATTCTGTTTTGCTGAGGTAGTGACCCAGAACATTCTGTCCTCAGCATCTGTGCACACACCCAGCCAGGTCTAGC
TTGGTGCCTGGTGACCATCAGTCAGAGATGTGCCTAGAGCTGCAAGAATCAAGAGGCATTCCCACCCACCCTTCAGGACATTTTTTTTTTTTTT
TCTGAGATGGAGTTTTGCTCTTGCTGCCCAGGCTGGAGTGCAATCGTCTGATTTTGGCTCACCGCAACCTCCGCCTCCCAGTTTCAAGTGATTC
TCCTGCCTCAGCCTCCCAAGTAGCTGGGATTACAGGCATGTAATCCTGTAATGGCACCACCATGCCCAGCTAATTTTGTATTTTTAGTAAAGAC
GAGGTTTCTCCACGTTGGTTAGGCTGGTCTCGAACTCCCGATCTCGGGTGATCCACCCGCCTCGGCCTCCTAAAGTGCTGGGATTACAGGCGTG
AGCCACTGCGCCCGGCCCCCTTCAGGACATTTAAACTAGCGCCCTGTGGCTGCCACAAACCAGCTGGCTTGAGTCCTAGACCGCCGGTCTTGGG
AAGAGGAGTATTCATCTTAGCTGCTACTAAACACCACAGTAGTAGTTAACCGCTATACAGTGCTTTACAGTGAAGCACTTTACATGTAGTAAAC
ACTTATTTCCCGCAACCCGATGAGGTAGGTACTGTCTCTATCACCACATTACAAACGAGAAGATAGAGGAAAACACACTCAAGATTACCCAGCT
CAAGGAATTGACAGAGTCTGGTTTGAGAACCTGTACTTTTTTTTTTTTTTCTTTTTTTGACACAGGTCTCACTCTGTCGCCCAGGCTGGAGAGC
AGTGCCACAATCATGGCTCACTACAGCTTCAGGCTCGGCACAGTGGCTCACAAATGTAATTCCAGCACTTTGGGAGGCTGAGGAGGGAGGGTTG
CTTGAGCCATAAGTTCTTGAACAGCCCAGGCAATATAATGAGACCCTGTCTTTACAAATATTTTTTTTTTTTTGAGATGGAGTCTCACTCTGTC
GCCCCAGGCTGGAGTGCAGTTTCGTGATCTCGGCTCACTGCAACCTCTGCCTCTCGGGTTCAAGCAATTCTCCTGCCTCAGCCTCCCCAGTAGC
TGGCACTACACGCGCGCACCACCACGTCCAGCTAATTTTTGTATTTTTAGTAGAGACAGGGTTTCAACATATTGGCCAGGCTGGTCTGGAACAC
CTGACCTCGTGATCCACCGACCTTGGCTTCCCAAAGTGCTGGGACTACAGGCGTGAGCCACCGTGCCCGGCCTACAAATAATTTTAAAAAATTA
ATCTGGGAGTAGTGGTGCACGTCTGTGGTCCCACTTACTCATAACGCTGAGGCAGGAGGATTGCTTGAGCCCAGGAGGTTGCCTTGAGCTGTGA
TTGTGCCACCACACCATGAGACCTTTTTCTGTGAGACTGTCTCCAAAAAATAAAAATAAAAAAAAATCATCACACCCTTGACCTCCCAGCCTCA
ACTGATCCTCCCACCTTAGCCTCCTCAGTAGCTGGGACCACAGGTGTGTGTCGCCACACCGGGCTAGACCCTGTCCTCTTAATGACTGCATAGT
CCTTGTAACCTCCATTTGCATTCCTAGAGCATAGCATTTGTTCCAAAAATATTTATTGAGTGTCTGTTCTGGGCTTGAACACGAGTAGGAGGAT
TTAAAAGTGAATAGCATATTGCGTCTCCCCTTGGGGACAGAGACAGAAACATGAAAACATGAAATTGTCCTTCAACACAAGAAGTGCTGTAATA
GAGGCATGAATAACGCGGTGGGAAACAAGGAGGGAGGTCATGCTGCCTGAAGCAAATGTCACCGAAGGGCAGACATCAAGAAGGCAGGACATTC
CAGGCACAGGGAATAGCATGTGTCCAGGGATGAAAGCTCACAAGAAAGAGAAACATGAGTCAGGTGCGGTGGCTCATGCCTGTAATCCCAGCAC
TTTGGGAGGCTGAGGCGGGCAGATTACGAGGTCAGGAGTTCGAGACCAGCCTGGCCAACATGGTGAAACCCCGTCTCTACTACAAAAATACAAA
AATTAGCCGGATGTGGTGGTGTGCGCCTGTAATCCCAGCTACTCAGGAGGCTGAGGAAGGAGAATCACTTGAACCCAGGAGGTGGAGGTTGCCG
TGAGCTGAGACCGCACCATTTCACTCCAGCCTGGGCAACAGAGAGAGACTCCGTCTCAAAAAAAAAAAAAAAGAAAAGAAAAAGAAAAGATAAA
AGAGAAATATGGACAACATGGAGCACCTGAGTGTCAAAACATCATGGGCAGTAACTGTCAGGGAGATTGCCAGAGAGCTCGGGGTTGGTTGGCG
CATCCATCCTGCTTCCTGCTTCCTTCTCTGGCCTCCAAGCTCCCTGAACCTACTCTGTGCAAAGCACTGTGCCAGGTGTTGGCCCAGAGTATGG
GACTGCAGCAGTGGCCTGGGTAGCGGAGAAGGGTTTTTTGACCAGCTGGAGACCTTTGTGGCTGGTGTCATTCTGGAGGTGAAGAGAAGGCCGC
TGGAAAGAGCTGCCCAGGGAGGACTATGGGAGAACTTGCAGCTCTGGGCCATCTCTGGACATGAACGTTTTGCATACACACAGGGGAGCTCTGT
GTGTCTGAGTGGGGGCGGGAGGGGTCCTTCTCTCTCTTTTGGCCCCTGCAGCTTGGCTTCTGGAGCTTTCCTATGTCTTGCTTTAGTGCTAGCC
CTTGGAGGGTTGCCAGGGAAACCGCTGGCCAGCTCCGGGAGATACACCTAGCACAGGAAACGACAGTGGGTGACTCAGCCTGGCTTCCCACAAC
CGCAACCCCCTCCTCAGCACGTGGAAAGGATCTGGGGGACCCCCTTCCCCTCGATCCTACAGATGACCCTGTTTATTCTTTCTTCTCTAGAGAG
AGCAGTCCCTCCCGCAGCTCTGACTGGTGGGAGCCACCCAGATTTTCCCAGGAAGGGAACCGGCCCCAGGCGCCCCGTCCCCCGTAGGTTCACC
CTGGAACTCCGACAGGTGAATTCGGCCTGGGCGAATGTGAATTCCTGCCGTGCCCATGCAGGCGGCAGGAGCCCTGGCTGAGACGCCCTCCCGG
GGGCAAACCCAGGATAATTATTGATCCGGGCCCCTGTGGACTTCTGACCTCGGCCTCGGCGGGAGAGGACCCGGAACGCGGGCCTGGGCTCCCA
GGGAGAGAGGTGGGCGGGCCGCGGAGGGGGCCAACCAAAACGACCACGACAGTGCGCCCTAGCCCTGCTAGTGGTGGTCGACCCAACCCAAACA
AAGCAGCATGTGCTTGCGGTGCGGCGTCGGCTAGGGAGCCGAGCCGGGAAGACACCCCACCCCCGCACAAAGGGGGTGCGCGCGGCGCCGGCAC
CGGCACCGCCGAGCGCGCCGCCACCTCGGGTGTGCCGAGGGGGTCCGGGTCCTTCAAGGGCGGTCGCGGAGGGGGCGCGAGGGACAGCGGGCTA
GCGAGCGCGCGCCGGGCGGAACCAGGAAGCGGGGCGCGCTCCGGGTTGCCAGCGGCGCGCGTCCCTGGGCCTCCGCGCGTGAGCGGCGCGCGCG
CCCCCGCGGGCCCCGGCGGCAGGAGAGTCCGCGGCCGGAGCCGTCACCCCGGGCGGGGACCCAGCGCAGGCAACTCCGCGCGGCGGCCCGGCCG
AGGGAGGGAGCGAGCGGGCGGGCGGGCAAGCCAGACAGCTGGGCCGGAGCAGCCGCGGGCGCCCGAGGGGCCGAGCGAGGTAGGAGCTGCGGGG
CGAGCCCTGGGCCCGGGGCCGGGGGAGCCAGGCCGGGGAGCCAGCCCGGGCGGGGCCCGCGCTCCAGCCGCTCAGCCGCCCGTCAGGGCCGCGC
CGCCGCCATCACTCCCGGGCCGCGGTGGTTGCGCGCTGGGCGAGGGGCGCGCGCGGGGAGAGGGGGTCCGGGTTGGCTGCCGGGGAGGCTGAGG
GCAATGCGGGGTGCGCGAGGGGGACTTGCGAACCCCGCGGCGCCGCGGGGGGCGTGCGCTTTAAAGGCTGCGTGCGCGTGTGCTGAGTGGGTGT
GTGGGGACCCCGCGCCGACCCCATAGGAGCTCAGGGGGCTGCTGTCAGCCGCCTCACACCTTCACGTTGCAATTCCGAATATCCCCAGCCGTCT
TCCTTGCTTTAACTTCTTTCTTTCAAAAGAGGTTCCCGCGACTCCGGGGATGGGATGAGCCTGCCGGGGTGCCTGGTGCGGTGGGGGCTATTAG
CAAAGCTTCAAGAAACAGGGCCTTATAAGGAATCAAGACAACAAATACGAACGTTTCGATAAGACTCTGGGGGGCGGCATCCCACATCAGGACC
CCCCAGCCCTGGGCTCGTGCGTGTGGCCGTCGGATGCAGTGTCCTTTTTCTTTCTAGTCAGGGATAAAGCCTTAAAAGTGCTCCTGGTGTCGGG
GGGTAGATATGGAGAGTCGGAAGGGGAGTTGTATCACAAAAGCTGGTCTGAGGCTAAAGGAATCTGGCAAGATGGTCGGGGTGAACAAAGAGGA
AAGAACTCACACGGAGCAACAGGCGGTTGCAAACACTTGTACCCAGCAGTGATGGAAAAGCCAAAGAAGAAAACCAAGTGTCAGCTTTTAGAAA
GACGGGATTAATGAAAAGCACAAGAATTTTAAAGACTAAAAGAGATACCGATTCTCTCTTTGCCAAAGACCCAGAGTCAGTTCTTTTCTTCTTG
GTCCCTGTGATCCCGCCATACCTCGTATCAAATAAATAGTGCCAAAAGTGGGTCAAAGAATGTTAGAGCTGGAAAGGAACCCAGCGATGATGCC
ATTCTTTTATTGTACAAATGAGGAAACTGAGGCCCAGATAGGTGGAGTAAGTGGCCTGCTCACGTGACACGGCTGTTTGTTGAGTATCCAGGGA
TTGAAACTTCAAGGACTCTGAGATTCGTGCTCTTCCCAAAGCATCGTGCAGTATACAGATCTTTGCCTACATACAATTCCATACATTTTCTCTG
CAGTGTCCTCAGGGAATGCCCTTTGAAAGGGCCAGCGTGACACTGAATATCATACTTGGGCTTATGTCATTTTTTTCAGTCTTCAAGTGGTGTT
TATGGTCTGCTTGGATCTTAGGTTTCCAAAGCTGTTGGATAGCCGCCTGGGCCATAATAAGCTTTCAGCTGTTGGGTCTACATTATTTCAAATA
ATTTTGTTATCTTTCAATTAAGGTAATAGAACTGTGCCTCTCTCCCCAACATTCTATTGTTGTCCATTTGCTTATGTTATATTGCGTTATGTTC
ATTGCCTGTGAGTTCCTACGTGAGGATGTATTTTATTGAGTGTCCCTTTCATGTTTTAAAATTTCATTTTGTAACACTGTAAATATTGGAGTGG
TTCTGATCACTTCAAATGGGGAGAATCTTATTGATTGATTTTATTTTAAGGCTTTGGGTTGTTTTGTTTATTCATTTGGGTACCAGTTTCCATC
TCATGGAAGATCACAGTTGTTAGAATGGACATGGCTAAATAAGAGAAGGGGCTGCTGATTTTAAGAAAAACTTCCTATGTGACAATTTGTGGTA
TCTGTTACGTTTAATTCAAACAAGTCCACACCTTGGGTCAGGTAAGCGCGTTATCTAGGAGATGACTTGTTTGCTACTGTAGCCTCTGGAAGAT
GAAATTTCAAGCTCCAGATTGAGACACAGGGAAGCAATGGAGAGGTTTTTTTTTTTTTTTTTTTTTTTTTTGTAAAGCAAACCATCAGAAAAAA
AAAGTTGATTTCTATCACCAGTAGTTTGGTTATTGCATAACTTGAAGCTAAACTCTGCACAAAATAATCTTTGCTATATACCCAACTAGGTAAA
CTATCCCACCTTCTCATCAGTTCTCACCTTTTCGGTGCTTGTATTTTTTGCCCACGGGATATTAATATGGATTGTGTTCAGAAATTTAAGGTGT
GTACTTCATAGTATGTATGATTGTAGGAGAAAAATGTGCTATCATCGTTTGGAGTGTTACAACATAAATATTTTGCTCAACACTTCCTATCCTG
CTCTGACTTTTCATCTCATCTCCATTTTCTTCCTCTCTTGATTGTTTTTATGTCATTTACATATGTAAAAGCCACATCAAGACTAAAGTTGCCA
TTTATTACTTTTTGATACTTAAGTCGAGTTTTTTCAAATCATAGTATTCTTACTATTCAAATTGTCTGTAATTTTTGTTAGGCTTATATATTAT
GAATCCATTTGTTGTGGTTTATTTGGCTTGTTGTTATGTAGGTAGGTGACACAGGTAGTCTTATTGTAAAAGGAAGACGTTATAGATAGGTAAA
TAACATGCTCAAGATTAGATGCAGGTTACACCTCCAATCGAAGGAAGCCTAACTATTTTCGTGCCCTACAAAAATGTCTGTATCTCATAAAGTT
ATTCACAGATTTCTGGACATTCTATCTCTGACACATTTTTTCCCTTTCTGTTAAGAGTGCAGATTACTGCATTATTCCTACAAAAGGAAGCAAA
ATAACCCTAGTTTGTATGATTTTTCTGATAATTTGAAACTTATTCGTTTAAAAAGCTATCTGGCAATTCCAGGTTACTGGACTCTTGAGAAGAA
TATCCTTTTATTTTGATACTATAAAATATCAAAGTATCAAAATACTTTAACTGTGCAACTTAATAAAAATAATGCTTCAAAGTTAGCTCTGCAC
TATCCCTCTCCTCAAATGTGTGGTATTTAAAGGAAGAAAATGTTTTTAATTCTGAGCTTTGTGAATAGAACTCTTCTAAAATCAGAAGTTTTTT
TTTTTCTCCTGCAGTGTTGGAAACACAACAGTTTTATGCTTATTTCTGGGAATAAGAAAGTCAAACTTAACAATCTAGTTTCCATTGATCAGAC
AGTTTTTGTACTTAAATTTAAGTTCTAAGCATCAGAGTGATAAATACTTTTTAAAATTGTATTCACTGTTAATTATAGTGGCCTTATTTGAAGT
AAAATCTTGACATTGGGTGTTTTATTAATGATGATCCTCTGCTACTGAAAAGCCTATGTCAGTTTGGAGAAGATTTTGCACATCTAAATCTATG
CTGTTTTTACTAATCAAAAGAGAAGAGCAATTTACAGGTGTTAGATTTTTAGTTGGTTGAATGAGACCCTCACTTTTGATGGTTTCAGAGTTGA
ACTGACGTTTTATTACTTTTGTTAGTTTGGGTCACACCCATCACTTCCTTGGAAGGTATCTTTGAGGTTTGGTTTCCTTTTTTTTTAAATGGTA
ATATTGGCGTCCCGGTGCTTCCTGTTTGTATTTTCAAAATTAGGTCCACATGTTAACCTTCTTGTAGTGTCACATTTTTATTTTAAACTTATAC
ACACAAACATCAGATGCCATGTGGTTTGTCTCTGACCCTTTGCGCTACTTCATGACAGCTTTACTGAGTGAAGCCTGAATACCCAATCGGGAAG
GCAAGATGTTCTTGTCTTTTCCCAACTCTCTTCAAAGTTTAACAGCCTTGGAAGGAGTCTTAGAACAGCAACAAAAGCACAATTTACAGAAAAA
TGGAGGTGCTGCTATCTCTTTCCTCGCAGGTACTTGGAAAATGTATAGCACTAAGATAGAGTTGAAACATGTTATGTAGAGCCTTGAATGTCGG
TGCTTCAAAGAGGAGTTGCTCTGGGTTTACATGATACAGAACCCCATGGTTTATATTAAGTAGTAATCATAGCCGTTTTTCTGGACTCGCTTAA
AACCCATACACAGTGTCATGGAAAAGAGTTAAGAACAAGGTGAAATTCAGGCTATGGTATTAGAGCTCAGGAGGAATCACCTTCAGCCTTAAAA
CTCTTCCTTTACTCTCCTGGATCACTTAAGTGATAGGTCACTTTTAATATAAATGTTGCAGGATGAGTTAAGTGGAGCCTTGAGAAATATGTGT
AGATTGTTTCAGTGATTAAAGCAAGTGTTCTTTTCCTGGCCTTTAACTCGCTTTATGCTAAGAAGTGTAATAACTAAATAGACTCCTAATTGAA
TTGACATTCAGATGTGTTTGCAGTTTACCCGGAGTTTACCTCTTCTAAATTTTCATTTGGATCCCATTACTAAAAGCAGTATTGCATTAGCATG
GTGCACAATTATGTACAATTCAATTTGCAAATGAAGTTAATACTTGGAGTATCTTGACTTCCTATTCCCCTCCTGCCCCAATATTCAAGAAAGG
AGAAGTTATAATTTGTCACATACAAGATGGTTTCTTCTTTAGGTTTATTCTTAATAACTATGTCCATTTTCACATGGATGACACCACTGAGATG
ATGAATACCTCTATTGATTGATTGATGAAGAACATTTATCTTTTTCTACCTACTTTTGGAAGCTAGCTTGCAAAATCTACTGCAACATTCACTC
TTCATGTATCCTTCTATTACCTTTGGTACCCTGAATAACCTTTTAAAAATTTTTGTACTTTTCTAATATGAAGAGAAGTAGCTTCATATTCTAT
GCTAGTTTTCTGAGGTGCTTGTGCTGACTCATTTTTTAAATTATACTAAGATATAGAGCATGGTTTGAGAGCAGTAATGAGATTCTCAGAAAAT
ATGAAAACTTTCCTTTCTGACTAGTTAGAGAAAGGTTGGGGAAGACAAGGGGAAATACATCAATGAGCCAATCAATTATTGTTATTTGTTGGGA
TTAATTTGGTGACTAATATTTTTAATGCCCATCAGGCTACTAAATGTGAATTTGGCTTTACCCTGTAATGATAACAGGACAAACCCAACGGGTT
TCTAACATTGCTTCTTTTCCCCTGGTAGGGGATTTCTTTGCATATGGGAAATCAGGCCGAAGTTGCCAGTAACAGCCACATCCATGATTTGTAT
TAAAAATAACTATTCATCATTCTCCGCTAGAGCTCTGCTTACCATGGCTCACCTGGGAAGATATTCTGATTATGTATCTAATCATTATGATGCC
ACCTTTCGAGAGGTGAGCAGACAATTAACAAGTCCTTCAAATTATGTTCAAAACCCAGAGTTTGAGAAAATGTTCCAACGCCTTCAAGTGTGAA
GATAATAAAAACATTACCTAATGCTTACCGTTCACTGACAGTGTGCTGGGCAGACATTGTTTTCAGCTCTGGCCTTGTGTTAACTCGTGATTCT
CAGAGGAGTGGGTATTATTCCCAGATGGAAGATGAAGAAATGGCCACAGAGAAGTTAAGTAATTTGTCCAAGGTCACATTACTGTTCTTGGCGG
GGCTGATTCCAGAGACTGCATCCCTAACTAGTGTACAGCAAAAGCGAGCTGGCCACTGCCACTTTGTTATAAGACATTTTGTTTCTTTGAGGCT
TGTATTTCTTTGTGTTCCCAAATGGAGTCTCGTTGGTTTTTGAATAGAAAATATTTTTATATGTTTAGGATTTTTTTAAATGTTGATGAAGATG
GTGATAAATATTTTTCCAATTAAAGTATATACTTCTGGTGGAAATTATATTAAAGAAGAGAAAGTCTTAATTTTTATTATAAGTTGTCTTTCAT
TCTTCTTCCCCTAGCATTACCGTGTACATCATAGAAGAAGAGCTATAAGCTGTCTCCTTGGCCTCTGTTTACAAACAAAGGGATCATTTGTGCC
AATGTAGTGGGTTTGTGTTGACATGCACGTCTGTACTGAGAACTCAACAATCCCACTCATTTTATTTACAACTTTATTTTGCGGGGATTTCTGT
CCTATTCTTGTTGCATTAAAGCTTTTTAGAGCATTAGTCATGATTTCTGAGACAGATTTAAGAATTTTACTTATTTGGGCTAATTTATTATTAT
TATTTCTTGAGACAGAGCCTCGCTCTGTCACCCAGGCTGGAATGCAGTGGCGCGATCTTGGCTCACTGCAACCTCCGTCTCCTGGGTTCAAGAG
ATTCTCCTGCCCAGCCTCCGGAGTAGCTAGGACTACAGGCATGTGCCACCATGCCCAGCTAATTTTTGTATTTTTAGTAGAGACGGGGTTTTGA
CATGTTGGCCAGGCTGGTCTCGAACTCCTGACCTCAGGTGATCCTCCTGCCTTGGCCTTCCGAAGTGCTGGGATTACAGGCATAAGCCACCATG
CCCAGCCTATTTGGGCGAATTTTTAACGCAACTGAACTATTGTATCTCTACAGCTCTGCAAAGGCCATTACAAAATAGTCTCAGATTTTTTTTT
TTTTTTCATTTATGATCCTTACTTTCTCTCAGCACTTTTCCTTGGGAGTGGATTGTGAAGTTAAGTCTTGGAAACAGAATTCACAACCCTAAAG
CGTAGTGCTTTAACTTTGGGAGATCAAGTCACAGACAACAAGAACTTCAATTCCCTGTGCAGAGTATAAACTTGTTCAAATAAACAGAGGATGT
TAGCTTCCAGGATTGTCAACTGTCACCTTTGACCAGGGTTGAATTTTCTTTGAATGTTGACAGAAGTTACAATGTCAGTGGAAAAAGCTTAATG
TGAGGTATCTATAACTCAGAATTTCCTTATGTTTGTGTCAGTCACATCCTACAATTAAAGCTGCTTCTTTTTTTCTATGTCTGATGAGTTTTAA
AAGCCTTCACTGGGGTTTTCCAGCCTTTAGGTGAGAGTAAGTTAAAAAGTTTGTGAGAAACTATTTTTTGTTGTCTAAAATTCAGAGCCTAGGT
CGGGCCCAGTGGCTCACGCCTGTAATCCTAACAATTTGGGAGGCCGAGGCAAGCGGATCACGTGAGGTCAGGAGTTGGAGACCAGCCTGGCCAA
CATCCTGAAACCCTGTCTCTACTAAAAATACAAAAATTAGCCAGGTGTGGTGGTGCACGCCTGTAATCCCAGCTACTCAGCAGCCTGACGCAGA
AGAATCACTTGAACCCAGGAGGCAGAGGTTACAGTGAGCCGAGATCGCACTACTGTGCTCCAGCCTGGGAGACGGAGCGAGACTCCATCTCAAA
AAAATAAATAAATTAAATAAATTCAGAGCCTATAGCTGGTGGCAAGTCACTGTTTTTGAAGGAGTGGGATTTTAAAAAATCTAACCTGGGCTAA
ATGTTAAGTTACATTCATACATGTCATTGTAGCCTATATAAAAAGAAACTTGAACACTTGATCTCTTAAAGTACTAAAAATTGGAATTCATCAG
CTTTGTAATATAGCATTCATTCTACACTATATTTAAATAGGTAAAATGTTCTGTTAGAAATAATGCTAATTCTGTTTTTTTGTTTAATGTTTCT
AAAAATCTTTTGGTCATCTTTTAGGTTTAGGAAAAGTAGCAAATTAGAGATCTACAGAAATCTACAGCTCTTTAAAAATGAAACAGTTCACCCT
GTGAATTTGTTTGCAGGATTGGTGATCTGTTTCTGATGCCAGGAAGGTGGGACTCCTATCTCATTTCTGATAGAAAGTGAGGGAAAGCTATGTA
TAGTGAGAACTGGATACTAGATTCTTATTCCGAAGGCATGGAAGGTTTAATATGTAAGGATACAATGTAAATAGTCCCTCTTTTTGTATTTTTC
TTTGGTTATTGTTACGTAACTATAGTCGTTTGTCACTTAATGATACATGGTGACAAAGTTCTAAGAAATGCATTAGGCGATTTTGTCATTGTGG
TACCATCATAGAGGGTACTTACACAAACCTAGATGGTGAGCCCTACTGCACACCTAGGCTGTATGATAAAGCCTATTGCTCCTAGACTACAAAC
CTGTGGAACATGTATACTGTATTGAGTACTGTAGACAATTGTAACACAAGGGTAAGTATTTGTGTATCTAAACATATCTAAATATAGAAAAGTT
ACAGTAAAAACATGGCATAAAGGATAAAAAATGGTACATCTATATAGCGTACTTAACAGGAATCGACCTTGCAGGACTGGAAGTTGCTCTGGCC
AACTCAGTCGGTGAATGGTGAGTGAATGTGAAGGCCTAGGACATTACTGTACATTACTGTAGATTTTAAAAATACTGTCTGGTTAGGCTACATT
AAATGTATTTTGAAAATTCTTTCTTTCTTCAATAATAACCTTAGCCTACTGTAATACTTTTACTTTCTAAACTTTTTACTTTTTTTTTTTTTTT
TTGAGATGGAGTCTTGCTCTTGTGGCCCACGCTGGAGTGCAGTGGCGTGATCTCGCATCACTGCAACCTCTACCTCCCGGTTCAAGCAATTCTC
CTTCCTCAGCCTCCCGAATAGCTGGGATTACAGGCGTGTGCCACCACGCCTGGCTAATTTTTGTATTTTTAGTAGAGACGGGGTTTCACCATGT
TGGCCACGCTGGTCTCAAACTTCTGACCTCAGGTGATCCACCCACTTTGACCTCCCAAAGTGCTCGGATTATAGGTGTGAGCCGCCGTGCCCGG
CCAACTGTTTACTTTTTTACACTTTTAAACTCTTTTGTAGTAACACTTAGCTTAACACATAAACATGTACAGCTGTGCAACAATATTTTCTTTA
TGTTTTTATTCTTTTACTTTTTCTCTTTTTAAATAATTTTTAATTTTTCAAAACTTTAAAAACTTTTTAATTTTTCAAAACTTTAAAAACTTTT
TCTTAAAACTGAAGGCATAAACACACACATTAGTCTAGGCCTACACAGCGTCAGGCTCATCAGTATCACTGTCTTCCACCTCCACATCTTGTTC
CACTGAAAGGTCTTCAGGGGCAGTAACACATATGGAGCTGTCATCTCTTGTGATAACAATGCCTCCTGAAGGACCTGCCTGAGGCTGTTTTACA
GTTAACTTTTTTTTTTAAGTAGAACCAGTACACTCTAAAATAACGATAAAAAGTATGGTAAATACATAAACCAGTAACATAGGCATTTATTATC
ATTATCAAGTATGTACTGCACGCAATTGTATGTGCTAGACTTTCACATGACTGGCAGTGCAGTGGGTTTGTTTACACCAGCATTGCCACAAACA
TGTGAGTCATGCATTGCATTACAACATTATGATGGCTACAGCCTCACTAGAGGATAGGAATTTTTCAGCTACGTTTTATTATCTTATGGGACCA
CAGTCATATATGTGGTCTGTCATTGACTGAAACGTCATTATGTGGTGCCTGACTACATATATGGAAACTCTGAGTGTAGTCATGTTTTACAAAT
GAGTTTATCTTGCAGTGAGTTTAAATATCAAGTAAAAATACTAATTTCATTGTAATGTATCAAGAGATTGGCCTTTCTCCTCATGTTGAAGGTA
TCAAAACAAAATAGAAAATATTTGGCTTCCTGAGTGGCATGGCTACAATTCACATGCTGAGTTGGTGATGAGGCAAGATCCCTTGACTTTGCAA
TACCAGAAAAATGCACATAGTTAAAAAAAAATAGAGACAGGGGTCTTGCTATGTCTCCAGGTGATCCCTCCCACCTCCACCTCCACACCTCCCA
CCTCAACCTTCCACAGTATTGGGATTACAGGAATGAGTCACTGCACCGGGCCAAAAAGCATATAATTTTAAGGGAATGATTTTTTTTTTTTTTT
TTTTATTTTTTGAGACAGTGTCTTGCTCTGTTGCCCAGGATGAGTGAAGTGTGCCAGGAGTGGGTCCTTAAGTAAGAAAACGTATTACTTTAGA
CACACATTTAAAATTTTGTCCAAAAATGTGATATTCTCAAAATTTATGGTTTTGAATTTTATTTATTATTTTAATTTATTATTATTATTTTTTA
AAGAGTGGGGTCTCACTATGTTGCCCGGGCTGGTCTCAAAGTCCTGAGCTCAAGTGATCCTCCCACCTCACCCTCCAGAGTAGATGGGACTGCA
GGTGTGAGCCCCTGAGCCTGGCTAGTTTTGAATTTTTATTACCCAAATGTGAAGAGGTCTAGTTTGCACAGAACTTAACATTACATTCCAGTTT
TATTGGTAAATTTGAACAGGATATAAGTGGCTTTAATAACTAATAAAAGAAGATACATCTTATAGATACACATATGTCTTTTTATATGTATACA
TAAATATTTAAAACTTCTTTATGCCAAAAAATGCCAGAAACCAAAGCTAAGTGACAAAATAAGAAAAATATTTCTAATATATATGACAAAGCAT
TATCGGTAAATATTCGTAATATATAAGTAATCTATATAAGTAGTAAGAAAAAGATGATTCCCATAATAGAAAATGAACAAAAGAAGAACTACAA
ATAGCCGATAATCATATGAAACAGTGTTCACCCATACTCGAAATCAAGAAATGCAAATAAAAATGACAAGTTTTTTCAGTTAACAGATTTATGA
AGACTAAAAATATTGAAAAATAAGAGCAGTGTTGCTTGGGAATGTGGGAAACTGGCACTTTCATACTCATTGGTTGAAATGTAAATGGGTGCTT
TTTTTGCATGGCAGTTTGATACATGTATTCAGAGCTTAAAAGAGTGCATACTCAAGCTGGGCATGGGAGCTCAGGCCTGTAATCCCAGCACTTT
GGGAAGCTAAGGCAGTCGGATCACGAGGTCAGGAGTTCAAGACCATCCCGGCCAACATGGTGAAACCCCGTCTCTACTAAAAATACAAAAATTA
GCTGGGCATGGTGGCACGTGCCTGTAATCCCAGCTACTCGGGAGGCTGAGGCAGGGAGTCAGAGGTTGCTGTGAGCCGAGATCACACTACTGCA
CTCCAGCCTGGTGACAGAGCGAGACTTCGTCTCAAAAAAAAAAAAGAAGAGTACATACCCTTTGACTTAGCAGTTTCATTTAGATGAATTTCAT
TCTAGGACATCTGAAATATGTTTAAAGATTTGTACAAATAGGCATGGGCAAGGACTTCATGTCTAAAACACCAAAAGCGATGGCAACAAAAGAC
AAAATTGACAAATGGGATCTAATTAGACTAAAGAGCTTCTGCACAGCAAAAGAAACTACCATCAGAGTGAACAGGCAACCTACAAAATGGGAGA
AAATTTTCGCAACCTACTCATCTGACAAAGGGCTAATATCCAGAATCTACAATGAACTCAAACAAATTTACAAGAAAAAAACAAACAACCCCAT
CAAAAAGTGGGCGAAGGACATGAACAGACACTTCTCAAAAGAAGACATTTATGCAGCCAAAAAACACATGAAAAAATGCTCACCATCACTGGCC
ATCAGAGAAATGCAAATCAAAACCACAATGAGATACCATCTCACACCAGTTAGAATGGCAATCATTAAAAAGTCAGGAAACAACAGGTGCTGGA
GAGGATGTGGAGAAATAGGAACACTTTTACACTGTTGATGGGACTGTAAACTAGTTCAACCATTGTGGAAGTCAGTGTGGCGATTCCTCAGGGA
TCTAGAACTAGAAATACCATTTGACCCAGCCATCCCATTACTGGCTATATACCCAAAGCACTATAAATCATCCTGCTATAAAGACACATGCACA
CGTATGTTTATTGCGGCAGTATTCACAATAGCAAAGACTTGGAACCAACCCAGATGTCCAACAACGATAGACTGGATTAAGAAAATGTGGCACA
TACACACCATGGAATACTATGCAGCCATAAAAAATGATGAGTTCATGTCCTTTGTAGGGACATGGATGAAATTGGAAATCATCATTCTCAGTAA
ACTATCGCAAGAACAAAAAACCAAACACCACATATTCTCACTCATAGGTCGGAATTGAATAATGGGAACACATGGACACAGGAAGGGGAACATC
ACACTCTGGGGACTGTTGTGGGGTGGGGGGAGGGGGGAGGGATAGCATTCGGAGATATACCTAATGCCAGATGACGAGTTAGTGGCTGCAGCAC
ACCAGCATGGCACATGTATACGTATGTAACTAACCTGCACAATGTGCACATGTACCCTAAAACTTAAAGTATAATAATAAATAAATTAAAAAAA
AAAGATTTGTACAAAAGGAGTGTTACTTAAAATAGCAATAAATTGGAAAAATCTTGGTATTTCCATTAGAGGTTTAATAAACTAAATCATTTAT
TACACCTGTGTATAGCTGTCCAATAGAATACTGTCCAGCTATTTAAATTATGTTATAAAACAATATTTAATGACCTACACAAAATTCACTATAT
ATTACTAAATGAAAAATGAAATTACAAACAATCATATACAATTGTGCCGGAAAAAAGAACAGGCGCTGAAAAAAGATTTCTTCTGGGTCGTGGA
GTTCCCTGTGTGTGTGTGTGTGTGTGTGTGTGTGTGTGTGTGTGTGCGCGCTTTCTAGTATCTTTCCATTTTTCTATAATAAACAGATTATGGC
TCCGCACAGTGACTCATGCCTATATTTCCAGCACTTTGGGAGGCTGAGGTGCGAGCATCACTTGAGTCCAGGAGTTCAAGATCAGCCTGGGCAA
CATAGCAAGACCCTATCTCTACAAAAAATTAAAAAATTAGCTGGGCCTGGTGGCACATGCCTGTGGTCTCAACTACTTGGGAAGATGAAGTAGG
AGGATCACTTGAGCCTGGGAGTTCGAGGCTGCAATGAGCTGTGATTGAGCCACTGCATTCGTCCTGGGTGACAGAGTGAGACCTTGTCTCAAAA
ACAACAAATAAAAACAAACAAAAAAACAGATGATAATACAGGGAATCCTTTTGTAATCAGGAAAAAAAAGTTATCTTTAAAATGGCTGTGTGTA
TTGATATGTCTTAGAACGTAGAATTTGTTTTTTGTTATCAGTGTTTTCTTTTTAGAAGCAGAAAAAAGAGTTTGTGATGTTTTTGTTAATTTTT
TGGTATAGTATCCTGTTTTTTGACATCTTTTGTTGAACTATGTTAAAATCATCATAATTTCATTTAACATTTTTAGAGTTTAAATAATTTGAAC
TTCAGGACTGTGTTATTTCCTGTGCTTCGTGGTAGCCTGTATATTTTTACACAGAAGCTTATGGAGAATTATATTGTTATATAATATAATAAAT
TGTTAGGGAGACAGAGGTGAAAGGATTGCTTGAACCTGGGAAGTGGAGGCTGCAGTGAGCTATAATGGTGCCATTGCACTCCCCAAGCCCGGGC
AATGGACAGAGACNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNATCTCTATAAAAATACAAAAAT
TAGCTAGACGTGGTGCTGTGTGCCTGTACTCCCAGCTACTTGGGAGCCTGAGGTGGGAGGATGGCTTGAGCCCAGGAGGCAGAGGTTACAGTGA
GCTGAGATTGCACCACTGCACTCCAGCCTGGGTGACAGAGTGAGACCCTGTCTCAAAAATAAATAAATAAAAATAAAAAATAAAAAATCTATCT
TAACTTGGATTGTGCATTGTAATTTATTATAGTGAAATTGCTATAACAGAATTTTATTTTAAGCCAGTTTGGGGAATACCAAAACATTTTTAAA
AATCACGTAGTACTTTTTAGTGCTTTTAAAACCTAAATCCATAAAGTAGTTAATTAAAATGCATGAAGTAAATAAGTGTTTTTCTGTATTAAAG
ATAAAACTATAAACATATATTGTTCAATATGCCTACATAAATAAATCATTCTCTTCTTTTTGGTTATTTTTTGCTTATCACCTCTTACCTCCTC
TACTTCCTAAAAACATGATAATCTCGTTTAATTCAAAATTATGTTTTCTGGCCATACATGGTGGATTATGCCTGTAATCCTAACACTTTGGGAG
GCTGAGGTGGGCGGACCACTTGAGTCAGGAGGTCGAGACCAGCCTGGCCCGCTTGGTGAGACCCTGTCTCTACTAAAAATACAAAAATTAGCCG
GGCGTGCTGGCACATGTCTGTAGTCTCAGGAGGCTGAGGCAGGAGAATCATTTGAACACAGGTGGCAGATGTTGCAGTCAGCTGAGATTGCACC
ACTGCACTTCAGCCTCGGCGACAAAATGAGACTCTGTCTCAAGAAAAAAAAAAAAAAGGTAAGACATATAAACTAGGAAACTAAGCAATAATTG
TGTACAGTATGTGGAATATTTATGTTCTAGACCAGCACTGTTCAATACAAATATAATGTGAGCTCCAATTGTAATTTTCTTAGTCTGTATCACA
ATAAAAAGTAAAAATAAAATTAATTTTAACAATATACTTTAATATATAAAAATTATCATTTCAACATTTGATCAATATAAAAATTATTGATTAA
TGAGGTATTTTGGGCCAGTTTGGGGAGCCAAAGCAGGAGGATCACTTGAGTCCAGGAGTTCGAGACCAGCCTGGGCAACATAGTGAGACCCAGT
CTCTAACAACAACAACAACAAAATTAGCTAGGCATGGTGATGTGCACCTATAGTCCCAGCTACTTGGGAGGCCAAGGTGGGAGGATCACTTGAG
CCGAGGAGGTTAAGGCTGCAGTGAGTCGTGTTCGTGCTACTGCATTCCAGCCTGGGCAACAGAGTGAGACCCTGTCTCAAAAAAAAACAAACGA
ACCCAAAAGCCCACAAAAACACTCACATATTTTACATTCCTTTTTTTGTACGAATGCTTCAAAATCTGGTGTGTATCTTACAGTTATAAAATCT
CTCAATTTAGCCACCAAATTTTCACCAGAAATGCATGAACTGTGTTCAGATTTCATAAAACTTTCAGTTGATAAAGTAGATTCACATACTGAAG
GTGTTCCAAACGTACTGAAAGGTTTTCCAAGTATTAAATTGAACACCAGTTTCCAGGTTTACATTTAAGTTAATTCAAATGAGATAAAATTTGC
AATTCAGTTCCCCAGTCGCACTAGCTACATTTCAAGTGCTTAGGTAGCACATGCTGCTAGAGGCTACCATAATAAACAGTGTGGCTATAGGCCT
TCTAACGAAGGGAAATGAAATATAGGGCTGTCTCCTGCTTTATAACATCTGGCTTACAATCAACAAATACAAATAGGGTCGCTTGCTGCAGCAA
ACCACCATGGCACATGTTTACCTGTGTAACAAATCTGCACATCCTGCACATGTATCCTGGAACTTAAAATAAAACAAAACAAATAGGGTCTCTT
GCCCTGTGGGAACTGAAGCCACTGCTCTGTAGGAAATAAAAGTGTCTTTCTTTTTTGTTTTTTTTTTGAGACAGAATCTCGCTATTTTGCCCAG
TGAAGTGGCATCATCTTGGCTCACTGAAGCCTCCCCCCGACAGGTTCAAGCAATTCTCCTGCCTCAGCCTCCCAAGTAGCTGGGATTACAGGCA
CCTGCCACCACGCCCCGCTAATTTTTGTATTTTTAGTAGATACCGGGTTTCGCTGTGTTGGCCAGGCTGGTCTCAAACTCCTGACCTCAGGTGA
TCCACCCACCTTGGCCTTCCAAACTGTTAGGATTACAGGCGTGAGCCACCGCGCCCAGCACAAAAGTGTCTCTTACCTAAAGAAAGTGATGCCC
TTAAAGCTTCCTACCATTCCCTCACCCCCAAGACACACCCAGACACCCACAGATCCACCCTCTTGTTTGAAAACTGCTGCATCCTTGCATGCCT
GAAGCATTACCCATGTCCTTGAAGCTCCTGCTTTCAGAGGACCTCTCATTTCTTGGGAGTAGAAAAGCCCTCTCTGGCCAGGTGCAGTGGCTCA
CGCCTGTAATCCCAACACTTTGGGAGGCCAAGGTGGGAGGATCGCTTGAGCCCAAGAGTTTCAAGTCAGCCTGGGCAACATAGTGAGATCCCAT
CTCTGAAAACAAAATTCTGAATAAAAATAAACAGCCCACCCTGTTTCAGGTATACCTCAACTGTCAAATCACTTTTAGGCAGGGTGTTTTGTTA
TTTTTGTTTTGTTTCAGAGTGTTGTTTAACTTTATTTTTGAAACTGAATTTGTCTAGAAAAGAATCATATTCACCTTTTGGTCTTAGCTACTCT
TCATTTTTATAAAATACATCAAGTTGATGCTTAAAATTGGGGATCTAGTTACAGATGAAATTTTTCCTTTCAAAAGAGAGTTCCCCCCAGTTTT
GCTGTAGTCTTAGGAGATACTTAAGGGAGGGGAATCATTTTTAGTTCCCCCTAGGTGAGGTGGTAGCCTCCCTAAACTGCCAAGTTCACACCAT
GACTGGTAACACCACTGTGTGAGGAATACTGAACAAACGCACCTTCAGAAGGGAGACTTCCAGAGATTCTGGAATCTAGGTAATGCCACTTCCT
ACTTAGTTTATCTTAATGGTTTTGTTTGTTTGTTTGTTTTGAGACGGAGTCTCGCTCTGTTTCCCAGGCTGGAGTGCAATGGCGTGATCTCAGA
TCACTGCAACCTCCGTCTCCCAGCTTCAAGTGATTCTCGTGCCTCAGCCTCCCAGATAGCTGGGATTACAGGCAAGTTCCACCATGCCCAGCTA
ATTTTTGTATTTTTAGTAGAGACAGGGTTTCACCATGTTCGTCACGCTGGTCTCAAACTCCTGACCTCAGGTGATCCTTCCGCTTCAGCCTCCC
AAAGTGCTGGGATTACAGGCATAAGCTACCGTGCCCAGCCTTAATGGTCTTTTTAAAGGATACCTGATTTCAGAAGGAACCTTTGAAGAAATGG
CTAGAACTTCTGTGTTTCTTTGGATATTGCCTGACATCTAAGCCAAATTAGTTGACCTTTTAATGTTAAATAAAATATTCAATAACTTTTACTA
ATGGGTGGGCTTTAGGAACCCCTTTATTTTACTATGTTGCCAAAAGTAGGTTCATTATAACATTGGAAACATCCTGATGTAATTTATTGAATGT
AAAATTATTCCTCAATACATTTGAGGATGATTTCCATAAAGGGAAACAAATTCCTTTCTTCCAAATATACTGTGACAGCCTATTTTGAAATATT
TAAAATATGTCCCTAAAGATGAAAATTAAAGAGCGGACACAAAGTTGTTATGCTCTAAGGAGTCATTCTTTTGATTTAAAATTCATTATAATTC
CCTGGTTGTCTAGTGGTTAGGATTCAGCACTCTCATAAAATTCATTATATTGATTCTATAAAAGTATATTGTGTTGGATTAAGAAACACATGGA
CATTTTTAAGTGTTTCAAAATATTTTTGTTATACCTAGGTTTTCACTGTGGTGGTATTTGTTGTTTTAGTAGAATTTGACCTATATTTTATTCT
ATGGGAAAAAGAAGAAAGTAAACATTGGCTAGCTATCTACCACCCTGTACTGCTATTTGTGTGTGGTCCCATTGTGAATTTTTAACTCCATGCT
CTATATTTTTAGTGCTTTGTGAAGATTGAATCTGCAAATCACCTTTGAAAGACAAAGGATTTGATTTGATTGTGGCAATCATGAAAGTTATCCT
GTAAATAAAACTGTATGTCCTGGACCAAAGCTGGAATAATGCCACACTGAAATGATCTTAACTGTGGAATCCTTGATTGGACCCGATTTGTTTT
GCACATACAATAGCTCCATAAAGATGATAGGCTTTCACCAGGGATGAACAGATGTTCTGGTTTTATCAGAAATTGCTTTTGCATCACCAAGACT
TCACAAAACCCAGTAATCCCCCTGTTTGATGTTGTGCACCTGCTTTTAAAACTTCTTCCCAGATACTGCTTTGAACACACAGACTTTGTCATTC
ACCTGTAAGATTCAATTGTCAGGTGGTATCTGTTTTCTCCCAACAGAAATAAAGAGAAGAAGAATAAAAACAAAAACTAGAAAGTGTAGCAGGG
GCACAATTATTTATTTAAACTTTAAAGTATTTAATATTACATACCAGAATCTAAGTTACATAAAGCATGTCTGTTTTGTTCACTGCCACATTCT
TAGTACCTAGAACAGAGCCCAACATGTAATAGTTACTCAATAAGTATCTGTTAAATAAGTATAGTAGTCCTGCCTTATCCCAAGGGATGCCTGA
AACCTCAGGTAATACTGAACCCTACATATATATATTGTTTTTTCCTATGCATATATAATTATGATAAAGTTCAATTTATAAATTAGGCACAGTA
AGAGATTAAGAACAATGATAATAAAATAGAACAAGTATGTAGTCTTTTTTTTTTTTTTTTTTTTTTTTTTTGAGACAGAGTCTCGCTCTGTCAC
CCAGGCTGGAGTGCAGTGGCGCAATCTCCGTTCACTGCACGCTCTGCTTCCTGGGTTCATGCCATTCTCCTGCCTCAGCCTCCCCAGTAGCTGG
GACTACAGGCATCTCCCACCACACCCGGCTAATTTTTTGTATTTTGTTTAGTAGAGACGGGGTTTCACCATGTTAGCCAGGATGGTCTCGATCT
CCTAACCTCGTGACCTGCTCGCCTCAGCCTCCCAAAATGCTGGGATTACAGGCGTGAGCCACTGTGCCTGGCCGAATGTAGTCTATCTTAAAAT
ACTGTATCTTACTATGCTGTACTGTGGGTAACAGAAACCATAGAAAACAAAATCTCAGATAAGGAAAGACTATTGTATTCCCCTTTAATCTCAA
TACAGCAGGTGTATCCAGAGAACTTCAACCCTCAGAAGGACTGAAAGTGTGCCTTTTACTGTGGAAGAAAGAAAAGAGAGGGGGCTGGGTGCAG
TGGCTCATGCCTATAATCCCAGTACTATGGGAGGCTGAGCCAGGAGGGTCACTTGAGCCCATGAGTTAGAGACCAGCCTGGGCCACAGAGTGAG
ACGCTGTCTCAAAAAAAGTTGGGGGGATGGGGGAGAGGAGAGAGGAGACAGCAAATATCTCTTTGTTTCCTTTCGATTTCAGCATTAAAGATAG
AAAAAATGTGAGTTTATTAGGCAAAAACTCGTTTTTTAACCCCATTTTAAAGATTCTGGAGTTACGTATATATATCATAAAATTTACCATTTTT
AACCTTTTATTTTGAGATGGAGTTTCTCTGTGTCACCCAGGCTGGAGGTTAGCTCATTGCAACCTTCACCTCCTGGATTCAAGCAATTCTCCTG
CCTCAGCCTCCTGAGTAGCTGGGATTACAGGTGCACGCCACCATGCCTGGCTAATTTTGGTATTTTTAGTAGAGGTGGGGTTTCACCATGTTAA
CCAGGGTGGTCTGGAACTCCTGACCTTAGGTGAACCGCCCTCGGCCTCCCAAAGTGCTGGGATTACAGGCGCCAGCCGCTGTGCCCGGCCATTT
TCACTGTTTTAAAGTACAGTTCCGTGGCATTAAGCACATTCACATTGTTGTGTGACCGTCAGCACCTGGTCCATCTCCAGAATTTTTTCATCCT
AAACTAAAATCCTGTATTCATTAAACAGTAATGAGAATAAAACTCTCCATTTGCCCCTCCCCGCAGGCTCTGACAACCGCCATTCATTCTTTCT
GTCTCTATCAGTTTGACTACTCCACGCATCTTATATCAGTGGAATCATACACTATCTGCCCCTTTGTGTTTATTCCTTTTTTTGCAACATAATT
CTTTTCGTTGGAGACTTCACAGTTTTTCTTTCCTTTGATTGTTTAGTTTTTGCTTTGTACCTAGACCTGGCCAGTGATGCATGTGCAATGAGTA
TGCAAAGACAAGTTAATTCAGCAGCTTGTTGCATACCACTAGGGAGTTATTAAAGCTTTCAAAATGTGATTCCTCATCTGTATGCCTGTGGCTT
ATCTACTCCAAGTCTTGCTGATAAACCTGCACTGAGAGTAATAGCAAAGATTAGTCTTTCTCCATGTTTCATAAAACAAATTGTTCTCATTCTT
ACAGCCTGATTATGAACTGGAAAATTACAGGTACATTGTTGTAATTGTTTATACAGACAATGAGACATACTCTCCACAGGGGACCTCAGCTGCA
GGGGAGGTTCTGATTGCGTGTCATTAAGAAACGACAGTTAAGAAACTTTTTTTTTTTTAGACGGAGTCTCGCTCCATTGCCAGGCTGGAGTGCA
GTGGCACGATCTCGGCTCCCTGCAACCTCTGCCTCCTGGGTTCAAGTGGTTCTCCTGCCTCAGCCTCCCAAGTAGCTGGGACTACAGGCGCATG
CCACCACACCCAGCTAATTTTTGTATTTTTTGTAGACACAGGGTTTCACCATGTTGGCCAGGATGGTCTCAATCTCCTGACCTCGTGATCCGCC
TGCCTAGGCCTCCCAAAGTGTTGGGATTACAGGCATGAGCCACCGCACCGACCTTCTTTTTTTTTCCATTTTTAAAAATTTTCTATTCTCATTA
CATCTTCCTTTCTCCCTACTTTTCCTTCCCTTCCCTTCCAGTTTGTCCTTCCCTCTCTCTCTTTCTTCCTTTTTTCTTTCTTTTTCATTCTCAT
TTTTCTGAGTAGTTGCTGAAGACACACCCTCACCCTAGGGGTCCAAATATAGATGAAACAATCATACCTCTCTAGCAGTTCACCTGGAGGGAGA
GAGAAAAAGAGAGTAAGAGAACCAAAATGAAGGGGAAATATTCTTAATTTTTTTTTTTTTAAGGACAGAAACTAACTTGTTGAGGAAAAAGAGA
AATATACTGGGAGTTCTGACTGATAGGAACAAAGAGAGGACAGCTTCGAAGGATGATTTGAAAAGTAATCTATGGAATTTAGCTTTAAAGCAGG
ATCTTGCCCTGTAGACTACTTTATTATTATTATTTTTTAAGAGACAGAGTGCAGTGGTGCAGTCATGGCTCACTGTAGCCTCAAACTCCTGGGC
TCAAGCCATCCTCCCACCTCAGCCTTCTGAGTAGCTAGGATTACAGGTACATACCAGCACGCCTGGCTAATTTTTTAATTTTTATTTTGTAGAG
ATGAGGTCTTCCTGTGTTGCCCAGGCTGGTCTTGAACTCCTGGCCTCAAGTGATCCTCCTGCCTCAGCATCCCAAACTGCTGGGATGTTGTGGA
CTCCTTTAGAGCCTCTTGAAATAGGGCTAGTAAAGTGATTTTCTACCACTGTATCTGTGCTCATTTACATTGCGCAGATCACTTCTGCTTGACT
TCATAGTTTCAACCTATAACATGAGGGGTCAACAGGTAGATTCGGAACCATCAGAGTTTAGGCCGAAGCCCATAGAATATGAAAGTTATTTTTA
AAATTTATTTTTACAATGTCTACAAATGCAGTTGTTAAAGTGCCAGAGACAGAAGTATTGACAATGAGAAATTATCCAAAACCAGGCCAGGCGC
GGTGACTCACGCCTGTAATCCCAGCACTTTGGGAGGCAAAGGTGGCAAGATGACTTGAGGCCAGGAGTCACAGCCTGCAATGAGTTGTGTTCAT
GCCACTGCTCTCCAGTCTGGGCCACAGAACAAGACCCTGTCTCTAAAAATAATCATCATCATCCAAAACCAAGACTGGCTCCTATTGGAAAGGT
GGAAATGGTTGTGATGGAATTCAGCAACACCAATACCTTGGTATTTCTGACTTTCAGGATGTTAGGAGACTCTTTCAGTCCTTCCTGTTTAAAT
ATGAATGGATATGTATTGCCATTATTGCCCTGCCCCTACATCTCCCAAATTTAGATTGCTAGTATTTATGTAAGTTTACTAGTCACAAATAGAT
TCTTGCATTTCCCATCTCTTGTCATAGGGCCCTCTTTGAGTTCAATATGCTTTTATTGGCACTTACTTTGGTTTTCTTTACAGCAGCAGCTGCT
AATTAGTGACAGCTTTTGAGCAAGCAGCTTGTCATTTTTGGCAGAGATAGGGGACATCCATAGCAAGTCAAGACTATATTTTCATTTGCTAAAT
TTACTTTGCTACATGGAAATACATCTTGGATAAAAAATAGTCTGTAGTCAAAGGTGGCTGTGTTTACTGAATTATCTTCCTGAGTTGGCATTCT
GTCCTGTAGTTGAAGAACTCACAGAGATGAAAAAAAGAAAGAAGAAAGAAAGAAAGAAAGAAAGGCTTCAACGTCTATCAGACTGATGCCATAA
GTCCTGAGTCATCCTTCTAACATTAATCTACATTCTGGGTATAGAACCTCATCATCTTGTTTTGGCACAATCATAAATCTTTCTGGATTGTAGT
TAGTGGCATACATGGTAACTGAATGAGTGAATTGTTGACAAATGTGCTTTTCGTTTATACTCAGCTTCTTTCTTGAGATATAATTAAAAGAAAG
GTTAGTTTGCTTAGAGAAGATCCTTTCTTTGGAATTTTAGCTCATCTTAGCATCATCATATTTCATGTAATTCTTCTCTTTGAAATTGCTGCAA
TAAAGTGTTAGCTCTATTTTACAAACTTGTCACGATGTTTATGAGGGTGGCACAACTTGCTATTATGTTTACCTTCATTAGCACCAATGATGAG
TCAAAACCTCCGTCATGGTTTCTATAAAGTGTCATTCACTCCCATTATTCCTGTAATAAGGATTCAGATTAATCACCTCCACAGTGTTAAAAAT
ATCCTGAAGGAAATAACTGACCTACCATCCATTTCCCCCAATTTAGGTCCTTTTTGTAACATGGAGGTCTATAAAAATTGTATTCAATACACAA
TTACTAAATACCAACCATGTATATCAAGTACTATATTGGGGAAATGAAAATGAATCAGAGAGACATGGTCCTTGCTCCAGGATGGCTTACAGTC
TATTAGTTGGCCAGCAGATAACTTAGTTGCCTTGGTTCTTATGTCCAGAGTGTAGGGTAAATTCTAATTCTAGAGGAGGAAAACATCCCATCTT
TTAAAAATAAGATTATCAAAATGATCAAAGACATTGGATACAAATATTGTACTGATTCTATTTCTACCTAAAGGTGTTAAAGATTGTTTGAGGC
ACTTTTCCTAGGCTTGTGGAACTGCCAGGCATCCTTGTCCCAAATTTCCCCTTGATATATCGCACAGCACTGTTAAATTCAGAAGTTGCCTTCT
GTATCAGAGTGTCTATTTCTTGATGGCATAGCTTGGAGAACCCAGATGATCTTCTAGCATTCAACTCATACTTAATGTACATCAACAGTAACAT
AGTTATTTGTATATTTTCTTTTCTTTTTTTCTTTTTTTTTTTTTTTGAGACGGAGCCTTGCTCTGTCACCCACCCTGGAATGCAGTGGCACGAT
CTCGGCTCACTGCAACCTCCACCTCCTGGGTTCGAGCCATTCTCCTGCCTCAGCCTCCCCACTAGCTGGGATTACAGGCACGCATCACCACGCC
CAGCTAATTTTTTGTATTTTTAGTAGAGACGGGGTTTCACCATGCTGGCCAGTCTGGTCTTGAGCTCCTGACCTTGTGATTCGCCTACCTTGGC
CTCCCAAAGTGCTGAGATTACAGGTGTGAGCCACCGCACCCAATCTATTTGTATATTTTCATTTGAACATTTATTAATTATAGTAGGAATTTAT
CATGTATACCTTGTCTCCAACCCAATGCACAGTAGCATCAGGTAAGTTGCTGTTTACTTATAGGTATCATGTGAACAGGTATCCAGATGGCAGC
TAACTTCAGTATCCTTTGTTATCCTGTCTTACACACTGGGGTGTTTTACAAGTTGCTTTCTCATTTCTATTAATGACATTTCCCCCTGTATGTA
AAATGTTTTCTGCCATTACAAAGCCTAAGTTTCAGATGTTCTGTGATAAATATTGGTGATGGATAATAATGTAACTAATGGAAGCCCCTTAAGG
AATTTGCGAACAAGTTTAACAAAGTTAACAACTCAGCTTCTTATAGGAAATGTTGCATTGCTAATTCTGACCACAAGTGGTCAGGATCTAGGTT
TTGGCCTTATGGGAAAGTTACCCAAGCCCAGGACAGTTCTCGTTATTCTCCTCCCACTCACTTTTACAATCTAAAGCTTTTGGCTAATTTCTGA
TTGATTTAAAAGAATGAAGGTGAACCCCCACCCCCCAAATATGGCATCCTTTGGTTGTGCTTGAAGGTTTGTCTTCTTAGTGCTAACCCTCAGG
GAAGGGAGTCTTATTTAAAGCCTTTTTTTTTGGATTGTCAAATAATCTTTTTGTATTCATTTGTGAATTGCATTTTATGATAATTCATTTTGTG
GCTCGTGTTATAAAGGAGTTAAACTGCAGGTATCACTGTCAGGAGAAACTCATTTGACTAGCTGATGATTTTTTTTTTTTTTTTTTTGAGACAG
AGTTTCCCTCTATCACCCAGGCTGGAGTGCAGTGGCATGATCTCCACTCACTGCATCCTCCGCCTCCTAGGTTCAAGTGATTCTCCTGCCTCAG
CCTCCCGAGTAGCTGGGACTACAGGTGCCTGCCACCACGCCTGGCTAATTTTTTGTATTTTTAGTAGAGATGGGGTTTCACCATGCTAGCCAGG
ATGGTCTCGATCTCCTGACCTCATGATCCGCCCGCCTTGGCCTCCTAAAGTGCTGGGATTACAGGCATGAGCCACCGCGCCCGGCCTCGTGATG
ATTTTCAATAAGTAAACCACTGTGGTTCACAGCCTGAGCTCCGAAGGTGGACAAAACTCCTTGAATATTTATTACTACTACTGTTATTATAATT
CTATTACTACTGCTGCTAGTACTTTATTTTTCTCATTTCCCATGCTTATTCCTTTGCTATTATTTAAGGTTTGGTGATGAAGTATAGGAAGGGC
TTGGAAATGAAGTCATTTATTTTGTTTCCAACACAAAACAGTTTCTAGTGTGGGTGTGTGGGTGGCTTAATTTAACATAACAAACTAATACGTT
TTTCCTTGAACTCCATACTATCTTCATTTCAAACTAGGTATTCTCAGAGTGGGGTTAAAATACTCTCCATCATCAAAGCCCAGACATTTTAGAA
CAAGAGCTTGTCTCACAAAAAGCCTGGTGTCTCTGTTGCCACCACAACCTGAAACTAACGAGTTAGAAACCACATAGAGCCAACGACATGGGGT
AGGAGCTCGTAACATTTTCTCGCAAGTTTTGTGTAGATAAGTTAAGCCGGGAACCGATCTGGTTGAGAAAATGTTTAAAATCATCATTTTCCCT
TCCTTCTCCTTCACTTTTATACTCAAAAGGGTCTAATTTTGCTCCTCTGGGAAGATTGAATGCGTAGGCTTTCATGTAGTTTAACACTCTCTTA
ATTTAAAGAAGTCTGGGTTTGGTGCAGTGGCTCACGCCTGTCATCCCAGCACTTTGGAGGGCCGAGGTGGGTGGAGCGCTTGAGTTCAAGAGTT
CGAGACCAGCCTGGGCAACATAATGAAGCCCCGTTTCTACTAAAAATACAAAAAATGAGCCAGGCATAGTGGCACACACCTGTGGTCCCAGCTA
CTCAGGAGACTGAGACAAGAGGATTGCTAGAGCCTGGGAGGCAGAGGTTGCAGGGAGCTGAGATCACGCCACTGCACTCCAGCATGGGTGACAG
GGTGAGACCCTGTCTCAAAAAAAAAAATTATATTTTCTATCCTCTTCCCCTTTTTGTTATGTGTTTTACAGTCTTTTCTCCATGTCTAATCTAA
GAAACTTATATATTCATTGTATATATATCTACTGGTTGTCAGCATTGAACCTTGCCTGTGCCCTAGCATGTTGCAGAATGACTGACTTGACTTT
ATTTTGAGAATAAGTTCAAAATTTATTCTTTTTTTATCAGTTGACTAGGGACCTTATCTGTTGTTGCATTTATTATCTCTAAAATACCATCTTC
TTTTTCTTTTTATTTTTTTTGAGATAGAGTCTCACTCTGTTGCCCAGGCTGGAGTGCAGTGGTGCAATCTTGGCTCTCTGCTACCTCCACCTCC
CAGGTTCAAGCAATTCTCCTGCCTCAGCCTCCCGAGTAGCTGGGATTACAGTCACGCGCCACCACACCCAGCTAATTTTGTATATTTTTGGTAG
AGACGGGGTTTCACCATGCTAGCCAGACTGGTCTCAAACTCCTGACCTCAAGTGATCCACTGGCCTTGGCCTCCCAAAGTGCTGGGATTACAGG
CCTGAGCCACCTCACCCAGCCTACCATCTTATTTTTAGATGGAGACTTCTGGACATTGGTATAAAATCTGGATGGTTCCAGTCCTGACCTTTTC
TTCATGTTTAATCTCACCTTCCTAACACTTCCACCACGTTGAAAAAGCGTAGCTCCATATGATCTTACTGCCCTTCGTTCAGTCAAGCGAGACA
CTGACATTGTGTAGATTCTGGGTCTCACATTTGCTTTGTTTTTATCTGGAGCTTTACTGGAAACTAAATGCTATTGCGTACTAGCTGCAGAAGC
AGGAGGGGAAGTTGGCAAGAATTCGCTGCTTTCTGTTTGGTCTGTCTGAGGCAGCTGACCTTTTGGAAGGAAATCGCTGCTCTTCGCTTCTTCC
GGTAACAGTGCCGCCTGTGAAAGAACACATACACAGAAGGGCTCTGCCGTTATCCTGGCTGATGCCTTGCAGCATAAGCTGTTAGTTAAGAGGT
GGGAGCCTTTCAGTGCATCTGTTGTGTGCTGTAGGAGCATGCACTCAAGTTCCTACACCATGCTAAGTTGTGTGTAGATCTACTATTAGAGGTA
TAGTGAGAGACGGTCTGTTACCAGAAAAAGTAGGTTGCTTTAGCAAAAGTAATTGCATTTTATTTACTCCCACTATGAGTGCTGTAGTAAATGT
GGACAATTATTTTGTGCACAATTTTAGTTCATCTTAATGTTAAATGTCCTGGTAATTGCTTTTTATACCTGGATAACCGTTGTTGTTTTTTTTT
TTTCCCCAGCAGGACAAACATTTTGATTTGCTAAGACATGAGCTCTTATTAAATGTCATTTCTTCTCAGAATTAGAATAAGATTTTTTTTTTTT
TTTTTTTTTTTGGACAAGATCTTACAATGTCACCCAGGCTGGAGTGCAGCGACACAATCACGGCTCACAGCAGCCTCAAACTCCTGGGCTTAAG
CAGTTCTTCTGCCTGTCTCCTGAGTAGCTAGAACTACAGGCATGCGCCACCACACTGGGCTAATTTGTAAATTTTTTGTAGAGATGGAGTCTCA
CTATGTCGCCTAGGCTTGTTTCAAACTCCTGGCCTAATCAGTCTTCCCATCTCGGCCTCCCAGAGTGCTGGGTTTACAGGCATGAGCCACTGTG
CCTGGCCCAGAATTATCTTCTTCTTTTTCTCATACATTTTTTTTTTTTTTAGAGAATGAGTCTGGCTCTGTCATCCATCTGGCATCCAGTGGCG
CAATCATAGTTCACTGCAGCTACTAACTCCTAGGCTCAAGTGATTCTCCCACCTCATCCTCCCAAGTAACTAAGACTACAGGTGTGCGCCACCA
CACGCTACTACTTTTTGTTTTGCTTTCTTTGTAAAGACAGAGCCTTGATATGTTGCCCAAGCTGGTCTTGAACTCCTGAGCTCAGGTGATCCTC
CTGCCTCAGCCTCCCAAAGTGCTGAGATTACAGCTGTGAGCCACTACACCTGGCCAAGATTTAGCTTCTTAACAAACCTGCTACTCGTTGGATC
CACTTTTTCTGCAGGACTTGTTTTACTTAAACACAGCTAGTAGGACACCCCACAACTTGGGATTATTTTTGGAAAGCTGTACTTGGGTTTCATA
TCACCCAGTAAGCAAACAAGGGAACTAACCAGCTGATGAAGCGTTTCTCTCTTAAAAGAGGAGATTTCAGTTTTGTATTGTTTTAACTAAAACA
TATTCAGTTCAGTTTTTATTTTGTTATTTGAAATGACATATTCAAGTTATGTCTAAGTAAAAATGAAACATTTTAATTCATGGCAAATTAAGAG
AACACAGCTACCTGGAAAACCGGGCAAACTGCCATATCCTTCTGTATTTAGTTTTGGTTCGGTGGATAGGTGTGCTTTTCTAAGGTATTATTGG
TCTCTTCCCTAGATTTGCTGTTGATGGCAGAGATGGTTGGGTGAAGGTTTGTGTTGATGTACATAAAAGACATAGAGATTAGTACAGAGAGAAA
CATCTCACGGCCCCCTTAGCAAGATTGCCCTGCAGTGCTTCTGTTCTTGGAAACTGGTCTGATCTGTTCCACTTTCCTGGGGTAATGAAGAATC
CAGGGGGAATACAAACCCTGCGTTCGTTTTAGGTATAGGTTCTTGGCACAAAATAAATTGATATCAACAAAACAGGAAGATTTCTCCCCGGTTC
TGTTGGCCTTTGAGTAGGAGCGAAGTTAGGAACCAACAGCTGAGGTTTCCTGTGATCTGCTCTTGTAATGATAGCTTTCTAGACACCTTCAATT
GTTACTCTTGAACTGTCAACGGCCTTCACGTAAGCCAGTTGTGTTCAGGTATGTGATTAGAGGTAAGGGATGTGTTTGTTTGTGTTTAGAAATG
GGGTCTCACTTTGTGGCTCAGGCTTGAGCATAGCGGCGTGATCATAGCTCACTGCACCCTTGACCTCCTGGGAGCGCTCCTGTCTCAGCCTCCT
GAGTAGCTTGCACTGCAGAAGTGAGTCACCATGCCCAGCTAGAAAGTTTTTTATTTTAAAGAAATTCTGGTACCGTTGTGAGTGACTTTCTGTG
TATTTCGTTCTATTTACAGTTGGTACAAGTTTGGTTAGTCACATCAAAGATGAAAGAGGGCCACTAATATATATAAATATATGTCAGAATTACA
AATTAATCTGCTATTAACAGTCTTAAATCACTATTTGAAAACACATTTAAGGCACTATATATTTGAAAGTGAAAGAATTGCATTTTAAAAAAAA
TTTTTTTATAATTCTGAATTTTTACTTTATAATTCTGAATGTTCTACTTAAAATTAAAATTTTTTTATAATTCTGAATTTTTACTTTATAATTC
TGAATGTTCTACTTAAATCAAGGCTCCCCAGGTAAGACAGTGTCATAACCTGGATATTATCATGTTAGGAAATATTTCTTCCTGTGTTTTATGT
CTCTTTAAACTTGGTTCCTGTGGGATAGTCAGTAGTACCAATTCCCCATTGATTATTAAATTTATATCTTAAGTTTTACATTTTAAACACAATT
TAATCAAAAACTTTACAAAAATTGTACCAAAAAGCCCCTTCACCTAAATGTAACAACTGTTTACACTTCTCTTTGACCTTTCTCTATTTGCCCT
CTTGCATACGTGTTGCATACACTTTTCATTTAACATTCTTTTATTTCTACTCTAAAAAAATGTGTCTACAGGCCAGGTCTGATGGCTCACATCT
GTAATCCCAGCAGTTTGGGAGGCCGAGGTGGGAGCATCACAAGGCCAAGAGTTGGACACCACGCTGGCCAACATGGTGAAACCCCATCTCTACT
AAAAATACAAAAATTAGCGCTGGGCGGGGTGGCTCACGCCTGTAATTCCAGCACTTTGGGAGGCCGAGGTGGGCAGATCACCCGAGGTCAGGAG
TTCGAAACCAGCCTGGCCAACATGGTGAAAACCCGTCTCTATTAAAAATACAAAAAATTAGCCGGGCATGGTGGTCGGCACCTGTAATCCCAGC
TACTCGGGAGGCTGAGGCAGGAGAATTGCTTGAACCTGGGGGGCGGAGGTTGCAGTGAGCCAAGATCGCGCCATTACACTCCAGCCTGGGCAAT
AAGAGCAAAACTCCGTCTCAAAAAAATAAAAATAAAAAATTAGCCAGGCATTGGTGGCAGATGCCTATGGTCCCAGCTGCTCAGGAGGCTGAGA
CAAGAGAATTGCATGAACGCGAGAGGTGGGCGTTGCAGTGAGCCAAGATCATGCCACTGCACTTTAACCCTGTTGAAAGAGCAAGACTCAGCCA
GGCGCGGTAGCTCACGCCTGTAATTCCAGCACTTTGGGAGGCCAAGGTGGGCGGATCACAAGGTCATGAGGTCGAGACCATCTTGGCCAACATG
GTGAAACCCTGTCTCTACTAAAACTACAAAAATTAGCTGCGCATCGTGGCGCGTGCCTGTAGTCCCAGCTATTCGGGAGGCTGAGGCAGGAGAA
TCGCTTGAACCTGGGAGGCAGAGGTTGCAGTGAGCCGAGATCGCGCCACTGCACTCCAACCTGGGTGACAGAGTGAGACTCCATCTCAAAAAAA
AAAAAAAAAAAAAAATTAGCCAGGCATTGGTGGCAGATGCCTATCTATGGTCCCAGCTGCTCAGGAGGCTGAGACAAGAGAATTGGTTGAACAC
GAGCGGTGGGGGTTGCAGTGAGCCAAGATTGTGCCACTGCACTTTAACCCGGTTGAAAGAGCAAGACTCTGTCTCAAAAAAATAAATAAATAAA
ATAAAAATTTAAAAATGCGTCTACATGGTTTTTATATTTCAACATAGCTTTTATATTTCATTTAACTGGTTGCACCATATCCCATTAGATTAAT
ACATTATAGTCTAATCATCCCCCTATTTTGGACATTTTCGTTATTTCTGTTTGTCCCGACGGTATATATCCCTGGGAATTGGCCTGAATCAAGG
CACTTGTCAAAGAAAATTTTGCTAGGTAGAAACTAAACACTGTAATTCTCAATGGTAAGAGGGAGGCCATGAACTTTAATAGGGAAAATATTGG
TAACATGTGTGGATTTATTAATAAATTGCCTGTTTTGTTTGTTTTTAACTTTCAAACAAGAGGAAAGGGATTATATTTCATTTAAGCAGTATAG
AAACAGGGTATTCTACTAGCTGAGGAGTTTTTTTCCTGAACTAAAAGATTTGAATTGTCCAAGAGACACATTCTAAGATGAGTTGATCTTGAAT
TACAGTACAAATATAAACCTTCACTAGAAATTCTGAGGCTGGCTGGGTGTGGTGGCTCACACTTGTCATCCCAGCACTTTGGGAGGCCGAGGTA
AGAGGATCACTTGAGCCCAGGAGTTCATGACCAGCCTGGGCAACATAGCAAGACCCCATCTCTAAAAAAAAAAAAAAAAAAATTAGCCGGGCAT
GGTGGCACACACCTGTCGTCCCAGCTACTCAGGGGGCTAAGGGAGGAGGATCACCTGAACCCAGGAGGTTCAGACTGCAGTGAGCAGTGATTAC
GCCATAGCACTTCAGCCTGAGTGACAGAGTGAGACCCTGTCTCAAAAAAAAAAAAAGAAAAGAAAAGAAAGAAAAAAATTCTGAAGGTAAGATA
GGACTACATGGCAGCTTCAAATCATTTCTTATTTTTCCGTAATGCACATCGTCATTAATTTTGGGCTACAGCAAGTGCTTTTAGTCATCTGGAA
AGTGATACAGATGTAGCTTCTTCCCAAAGGGATCGCAAATCCATTATCTCCATGGATTTGGACATGGACATAATGATAACACATGTACACTGTG
GGCCACCATGGCACAAGACAGCCCAACTTGCATGCTGGTGGCCTGCAAGCTGCAGCACAACCCCTAAGAAGTCCAGATTTTTATTGACCACCAG
GGACAGTTTCAAAGTGAGAGTCAAACCTCTGCTTTCATTGCTTTGTTTTTCTTTTTTGGAATGTTTGCCCTTCATTTTGATTTTGAAAAAAGTG
TACATGACTTAAAAGTGGAAAGAATAGAACAATGAGTACTCGCATATACTTCACCTAGGTTCAGCCAGTGTTCACAGTTGTTAGATTTACTTTC
CCACACTCCCCACCACTGTCACTCTTCCGTTTCTCCTGCCAAAGTAAGATGCAGACGACATGGCCATTTACCCTCAAATACTTGACAGTGTATC
TACTCCCAGCAAGGGCATTCTTCTACGCAACCTGAATGCCATTGTCACTCTCAAGAAATTTAGCAGTGATAAAGTAATATCTAATATATAATGA
TATAATGGCCTTTAGAGCTGTGGGTGTTTTTGTTTGTTTTGAGACAGAGTCTCACTCAGTTGCCCAGCCTGGAGTGCAAGTGGCGCAATCTCAG
CTCACCACAACCTCTGCCTCCCGGGTTCAAGAGATTCTTGTGCCTCAGCCTTCAGAGTAGCTGCAACTACAGGCGTGTGCCACCACGCCCAACT
AATTTTTATATTTTTTTTTTATTTATTTTTTTTAAGTATAAACACATATATTTTTTAAAGTTTATTTTTAACATTTTTTTTTTTTAATTGATCA
TTCTTGGGTGTTTCTCGCAGAGGGGGATTTGGCAGGGTCACAGGACAATAGTGGAGGGAAGGTCAGCAGATAAACAAGTGAACAAAGGTCTCTG
GTTTTCCTAGGCAGAGGGACCCTCTGGCCTTCCGCAGTGTTTGTGTCCCTCCGTACTTGAGATTAGGGAGTGGTCATGACTCTTAAGGAGCATG
CTGCCTTCAAGCATCTGTTTAACAAAGCACATCTTGCACCGCCCTTAATCCATTCAACCCTGAGTGGATACAGCACATGTTTCAGAGAGCACAG
GGTTGGCGGTACAATTTTTGTATTTTTAGTAGAGACAGCGTTTCACCATGTTGGCCAGGCTGGTCTCGAATGCTTGACCTTGAGTGATCCTCTG
GGCTTGGCCTCCCAAAGTGCTGGGATTACAGGCATGAGCCACCACACCCAGTCGTGTTTTTGTTTTTGAATTACGCTCTTGGTTGTGTCTCTTC
AGTCTCCTTTAACCTAGAACAGTTCCACCTGTTTTGGGTGGGGGGTGGGATCATTGATTAGAGTCTCACAATTTGGATTTGTCTGATTGTTTCT
TCATGATTTAGTTTGTGTATTCTTTTCAGCAAATCATATCTGGAGGCACATAATGTCAGTTTATGCCATGATTGGGGATGTTTGATCACTCGGT
TAAGGCATTGTCCGCCAGATTTATCCACAGAAATGCACCTTTTCCCCTTTGAATAATAAGTAATATGTGAGTAGTACTTTGACACTAAATGCTG
TTCCCCAACAACTGTCACCTAATGGTTTTAGTAGCTGTGATGATCCTTGTCTCAGTCTACTCTCTTTATTTTTGAGATTGTGCATCTTTGGCCA
AGACTTTATATCCTATATTTAGATGAGAGTTTTAAATATATAGTAACCTGCTGATTGAAACCTGATTTTTATATTTAGTAAGTAGATCCTTTCT
CTACCTTCTCCTTTATTCCCTCCCTTACCCTTTTCCCCTCCTCCATTCTCTGTTCTTCCTATTGTAATATGCAGTGATTTGGCTGTGACATAAA
GATTAATGGCGCATTGTCTTCCCACAATACTATCATCTCTATGTTGCCTGGGCACATTTTTCCTTGTTTGCTTCTTTGTGAGCCAAATGCTTTT
ATTCTTTCTGTTCTCTTCCCATCTGTTTAATAATGTGATTTTTTAAAAAGAGAATTGGCTTCCTGATTGCATATAGGGTAGTATTTAGAAAAAA
AAAATTAAAGAGCAGATGGCTCTGGTATTAACCTGCTTCTAGATGTGATAGCCTTCTTACTTGATATTAGTCCAATTTTAATATGAATGAAGTC
TTAATCCTTTATTGCTGCTATTGAATATTTTTCCGTCCCTCAAAATCGCTATACTAGGCTACGCATGGTGGCTCAGCCCTGTAATCCCAACACT
TTGGGAGGCCGAGGTGGGTGGATCACTTGAGGTCAGGAGTTCAAGACCAGCCTGGCCAACATGGTAAAACCCTGCCTCTACTAAAAATCCAAAA
ATTAGCCAGACACACTGGCACATGCCTCTAATTCCAGCTACTCAGGAGGCTAAGCCAGGAGAATCTCTTGAACCCGGGAGGCAGAGCTTGCACT
GAGCCAAGATCATGCCACTGCACTCCAGCCTGGGTGACAGAGCAAGACTCTGTCTCAAAAAAAAAAAAAAATTGCTGTACTAGGTGACCACCAC
CATTTAATATATTTAATAATGTTGGTATCAACAGAAAGCAGTAAACATAATTAGGGAATTTGCTCTTTTTTTTTTTTTTTTGAGATGGAGTTAC
ACTCTTGTTGATCCATCAGCACAACTGGAGTGCAATGGTGCGATCTCAGCTCACCGCAACTTCTACCTCCCAGGTTCAAGCGATTCTCCTGCCT
CAGCATCCCAAGTAGCTGGGATTACAGGCATGCACCACCACGCCTGGCTAATTTTTTGTATTTTTAGTAGAGACAGGGTTTCTGCATGTTGGTC
AGGCTGGTCTTGAACTCCCAACCTCAGGTGATCTGCCCGCCTCTGCCTCACAAAGTATGGGATTACAAGCATCACCCACCACGCCTGGCTGGAA
CTTGCTCTTTATATTCTGTACAGAATCCTTAGTCATTCACTGGAGCTGATAGGTTTAACAGTTTTGAACCCTGGCTCCTTGCCTTGTACTTTTC
AGATCAGAAGCAATCTTTAAACGTCATCTCTTTGATCTGTCACCCAGCCCTAGCCTTTGCCAAAATCAGAGCCCCTCAGTATTAGGTTTCTTAC
TTTAATTTTTCATTCTTTGCTCTCTTTAAATTATGAAATATAACAAACAGGCAGAAAAGTACATAGAAAATGGCAAACCACCTTAAGTCCTCAA
CATGCATCCAGCCTGGTGCAGTGGTGCAAGTAAGTGCAGGTTCTCAGGTAGGTCCAGCCCAGATAGTACCCCTTAGTAGTATGGACCGACCCTG
GAGCAAGTGTTTCACCTCTTTCTCTTTTTAAAAATACCTTTACTGAGATATAATCACGCACTATAAAATTCACCCTTTGAAGTATATTCTCAGA
GTTATACAATTATCACCACTATCTAATTCCGGAACATTTTCATCATCCCCTCAAAAAGCCCTTACCTTTTAGCAATCATTCCCCATTTCTCCCT
CCCCTCAGCCCTTGGCAACCAACAATCTATTTTCCGTCTCTATGGATTTGCCTATTCCGGATATTTCATAAAAATGGAATTTACAGTATGTTCC
CTTGCATGCCTTTCACTTTACATAATGATTTCAAGGTCATCCACACTGTAGCATGCGTCAGTACTTCATTCCTTTTTAACATCTGCATAATATT
CCATTTTATTCATTGTATTTATCCATTCCTATCCATATTTATCTCTTTATCCTTTGCTGGACATTTGAGTTGTTTCCACTTTGCGGCGATTATG
AGGTGATTATGAATAATGCAGCTGTGAACATTTGTGTACCTGTTTTTTGTTTTGCTTTGTTTTTGAGACAGAGTCTTGCTGTGCTTGCCCTGTC
ATCCAGGCTGGAGTGCAGTGGCACAGATCTCGGCTCACTGCAACCTCCACCTCCCAGATTCAAGCAATTCCCCTGCCTCAGTCTCCCAAATAGC
TGGGATTACAGGCACCCGCCACCGTGCCCGGCTCATTTTTTTCTTTTTTAATTTTTAGTAGAGATGGGGTTTCGCCATGTTGGCCAGGCTGGTC
TCGAGCTCCTGACCTCAGGTGATCCACCCGCCTCAGCGTCCCAAAGTCCTGGGATTACACGCGTGAGCCACTGCACCTGGCCTGTGTACCCACT
TTCTGGGGACACATGTTTTCACTTCTCTTAGGCATATACCCAGGAGTGGAACTACTGGATAATGTACTAACTCTATGTTTACCCTTTTGTAGAA
TTGCCAGATAGTTTTCCAAAACTGTTTCACCATGTTACAATTCTACTTAATCTTGATGACCCTCAATTTCCTCATCCATAACGGGGATAATAAT
AGTGCCTACCTCTGAGCGATTTAGGATAAAGAGTAGATTCATAAAAGCACTTGTTATAACTAATATACTAAGTCCTTAGCTAGCTCTTAAATCT
GGATGGGGGATAATAATAACACTGATAAATGTTATTTATTTCTTATCCTATTTCTGAAGTTTATTTTTATTTATTAGATGAATTGAAATATTTG
TTGCTAATAATTCATAAAGCAGTATAATAAATGAAAACTTCAAATTTCATTAGTTGTTTTTTGTACTTTAATTATTAAAAGTCAGATATGTCCT
TGGAAGGAATAGGTAAAGGCATGGCTCCCTGCTGGCTTAGTCCTCTCTGTTCAACAACATATTAGAAGGAATGGAATAGGTAGTTTTCTTCTCC
TTTTTTTTTTTTTTTTTTTTTGAGACAGAGTCTTGCTCTGTCGCCAGCTTGGAGTGCAGTGGCATGATCTTGGCTCACTGCAACCTCTGCCTCC
TGGGTTCAAGCGATTCTTCTGCCTCACCCTCCTGAGTAGCTGGGACTACAGGAGCATGGCACCACGCCCAGCTAATTTTTGTATTTTTAGTAGA
GATGGGTTTTCACCATTTTGGCCAGGATGGTCTTGATCTCTTGACCTCATGATCCACCCACCTCGGCCTCCCAGAGTGCTGGGATTACAGGCAT
GAGCCACCGCACCCGGCCTCTTCTTTTTTCTTTTTTTTTTAAGAGTTTACTATGCATACGGCAAAATCAAAGCCTTGCTGTTACTGTACCACCC
TCACCCTTCACAATTCTCCATTCTTTCATTCAAAGATCAGACAACACACTTCATCCTGCCTTTCCACCACCACCTCCCAACAGTTCCTCTCTGC
AGAAGTCCTTGTGCCAGTGAAATTGATTAAGAAATAGGAATGCCATCTTTTAAAACGGCCCAGCACAGCCTGTAATCCCAGCACTTTGGGAGGC
CGAGGTGGGTGGATCACAAGGTCAGAAGTTCAAGACCAGCCTGGCCAAGATGGTAAAACTCCATCTCTACTAAAAATACAAAATTAGCCAGGCG
TGGTGGCGGGCAGCTGTAATCCCAGCTGCTTACGAGGCTGAGGCAGATAATTGCTTAAACCCGGGAGGCGGAGGTTGCAGTGAGCCCAGGTCAC
GCCACTGCACTCCAGCCTGGGTGACAGAACGAGACTCTGCCTTAAAAAAAAAAAAAAAAAAAAAGGCCAGGCACAATGGCTTCTGCCTCTAATC
CCAGCACTCTGGGGGAGGCTGACTTGGGAGGATCACTTGAGGTGAGGAGTTTGAGACCAGCCTGGGCAACATAGTAAGACCTTGTCTCTACAAA
AAAAAAAAAAAAAGTTTTTTTCTTTAATTAGCCAGGTGTGATGGCATGTGCCCACAGTCCTAGCTACTTGGGTGGCTGAGGTAGGAGTATCCCT
CAAGCCCAGGAGTTTGAGGCTGCAGTGAACCATGATCACACCACTGCACTCCAGCCTGACTGATGCAGTAAGACCCTGTCTCGGATGGATGGAT
GGATGGATGGATGGATGGATGGATGGATGGATGGATGGATAGAAAAGTTTATACTGGAGGATATTGGTTAAGGAGAAGGATAGCAAGAGCTATT
TGTGGAGGATTGATTTTTGGAAAGTTTATTAAATTGAGTTAAAATTAGAGATGGGAGAAAGAAGCCGGTTTTGTAAGTTTAAGGTAACAGTAAG
AACAGTAGTGAGTACTTACAGAGCACCTGCTCTTAGCTAGATACTGTTTTAGCTATGTACATAAAACTAATTCATTCTTACACCAGCCCCCTAA
GGGGCATAAGTATTATTATTCCCATTGTACAAATAAGAAAACTGAGGCACAGAGAGGCTTAACAGGCTGTCTTTGGCAGAACCGTATTTAGGTG
AGTTCAGCATCATCTCTAAAAGTTTAACCCCAGTTTAGAGACTTTGGCCCTGGGATGGGGGGTACGATGGAGAATCGGAATTACAAAATGCTTT
AAATTATACATATGAATCCAGCACACCAAATCTGCTGGATTCATATTATTCATATTATGCATAATATTATTCATATTAACCCCCTACTCCCACC
CCACACTAGAATTCATTCACTGAGTTGGCAGATACACAGTGGGCCTTCTAATGTCAGACACTACTCTCCACTGCAGTTTAGCTAGGGCAAGACC
TTGGTCACAGTGTTCTACAAGTCTTCAGAGAGAGAGTAATGTTTTTCTTTGTTATAGTTTATATTTTACTTTTAGAAAGGAAAAGCATCTGAGA
AAAGATATTTATGACTGAGACAAAATTATTCCAAATCCCACTCACTTATATCATTTATTTATGTGGTGGACATTCATTTTTATGGTTTTCAGTA
AGCCTGTAACATTATGAGCTATTAGACTTTGAAACTTTTATTAACAAGTGCCTGAGGGCCCGACTGATTTATTTGGCTCTCCAAGCCCATGGGT
ATATCCAGATATATAAGGTTGAGGTAGGTGCTTTATGCCTTCACAAAGTACACGGTTCTGCATCCAGGCCTGTCTTAGGTTGTTCTGAAATGTC
TGTGAACACAGAGTGCAACTCAGCCCACCTCAAATACTTCTCATCTTTGGCCTAAAAATAAGATTGTGAATCCATTTCCCAGTCTTTAGAGAAT
CCCCTGTGATGATTTACCAAAGAACAAACTACTTCCCTTGTCTGAGCATCACGCACTGAAATTGGAGCTGCCCGTAAATAAGCAGAAGATGGCG
GCTACTGGGGACAGAGGTACAGTCTTTGTAGATGCAGAGTAGCGTTCGATCCCTACATCAATGCCAGTTAGAACGATCAATGCCGGTTAGATGA
ATGCTACTCATATCAGGCATGTGGACATAGCCTGATAATGGGGTGAAGAGTTGTGACATGTATGAAGAAAAATACTACTTAACTGCCAGGCACG
CTGGCTCACGCCTGTAATCCCAGCACTTTGGGAGGCCGAGGCGGGCAGGTCACCTGAGGTCAGTAGTTCAAGACCAACCTGGCCAACATGGTGA
AACCCCATCTCTACTAAAAAAAATTAGCCAGGCGTGGTGGTGAGTGCCTGTAGTCCCAGGTACTCAGGAGGCTGAGGCAGGAGAATCGCTTGAA
CTCGGGAGGCAGAGGCTGCAGTGAGCCGAGATCATGCCACTGCACTCCAGCCTACGCCACACAGTGACACTCCGTCTCAAAAAACAAACAAACA
AAAAACCACTACATAGCTTCTAGGTGTTATTCACGTAACTCGGAGAACAGTTGGTTTTTTCCTAAACTGGGTGTTTAGAATTTATAATGATAAT
CTCAGTAAGTCATCTCATATTTCACATATAGATAATTTTCAGTTTAACAGTAAAATAAAAATGACTCTGTACTAGTTAGTGTACTTTTATTTTA
CACTTTTGAATAGCAGTGAGCAGAATCTACTCTCCAGCTCTATCAAAGCACTTATGTGTAGTGAAACTATATTGTATTTATCAAAATAAGGTGA
TTGCCACTTGGTATTTACCACCTATTATCCTTCAAATGGGATGCTATTTTAGAAAATAATTATTATGGCATGTTCATAACTAGAATCATAAAAA
TATTCTACAAATTCTAAGAGCTCAGCATTCTACTCTTTGATGAAAACTAAGAAATAATCTAAAATTTAAGGCAAAATAGAGTGACATTTTTTAA
TACCCCTACATGCATAGTATATCACCACAGTATTTCTTTCCTAAAACAGTAATAGGCTCCTAAATATGCTTCACCACCAGGGAATGTTAAATAT
TTGTTTGTGTATTTATTCGCTCCCCAAGTATTTTTTGACCACTTTCCATAGGCAAGCCACCGTACTAGACACTGGGGACACAGTCGTGAATAAG
ATGTGCTCCTGGCCGAGCGTGGTGGCTCACGCCTGTAATCCCAGCACTTTGGGAGGCCGAGGAGGGCAGATCACGAGGTCAGGAGACCAAGACC
ATCCTGGCCAACATCGTGAAACCCCATCTCTACTAAAAATACAAAACTTAGCTGGGCGTGGCGGTGTGTGCCTGTAGTCCCAGCTACTCGGGAG
GCTGAGGCAGGAGAATTGCTTGAACCCGGGAGGCAAAGCCTGCAGCGAGCTGAGATATCTGCACTCCAGTCTGGGCGACAGAGCAAGACTCCGT
CTCAAAAAAAAAAAAAATTGTAATTGATTATTTAAATGGAAATCTGTTCCATTTTCATGCCTTGGTGTTTCATACTGATATATTGTTCAATTAT
AACTTTGAAAAGAGCCAATAGAATCGGTCAAATTGTGTGTATACATTTATTTATGCAACAAAAAAATTATGATGCTTATGTGCCAGCTACCGTT
CTAGACCCAGGAGAAAGCACGGTGAACAAAACAAAGTCATTGTGTTCATGGTGATTTCACCCTGGTGGGAAGTCAGTAAGCAAATATATGATCA
ATATCACGAATTGATAAATGATAAGAAAAATAAAGCAGATTAAGAGGATAGAGAGTGACCAGAATGCCGTTTTATTTCAGTGGGCTGGCAAGGA
GTCTCTGGGGAGATAAAAGTTTTCATAGCTTTCCTTCTTTTGTCTCTGAAGTCAATTCACCTAATGAATGCAGATGCAAGAGAATTTTTGTATC
TGTTGGTATCACTCACATATTCATTGAGAGCTATGCTGACGCCCTACTTCTTACTCTTTACTACTTAGGAATACTAACACCTACATTCTTCTCT
TCATTCTTTTATCACTCTTCCATATACTTAAGTGCCTAAGCACCTTCATTCATCCATCCCTTATATAATTTTTTTTCTTGAGACAAAATCTGGC
TCTCTTGCCAGGCTGGAGTGCAGTGATGTAATCTCGGCTCACTGCCACCTCTGCCACCCAGATTCAAGCGATTCTCCTGCCTCAGCCTCCCAAG
TAGCTGGAATTATAGACGCATGCCACCACACCTGGCTAATTGTTTTGTAATTTTAGTAGAGACGGGGTTTCGCCATGTTGGCCAGGCTTGTCCC
GAACTCCTGACCTCAGGTGATCCACCCACCTCAGCCTCCTGAAGTGCTGGGATTACAAGCGTGACCCACCATGCCTGGCCATCCATTCCTTATC
CTTCATCCAAAAATCATGATTTGGTATCTGTTCTGGTTTTCCAAAATCTTGCGATATGATTTATTTGATTAAAAAATACATTGTGCAGTTAAGT
GACTTCCAGTGTTTGATTATAAAGTGGAATTGCTTCTGGAGAGAGCTCTTTTATCTAAGTGTTTTGGTGAACTCACAACCACTGAAATTTGATG
TCTTGGGTCAAATTGGGTCTACAGTGATCCCAGCCTTAATGCCTAAAAGCCCTAAATAATACACTTCTCAAGTGTTCTATAGCATTGCACTAGT
CTAAAATACATAACTGGCACTCCCCTACACCTCATTGTTGGCTAACTGACTTGTCCCATAAATAGCCCAGTGTTACGTGATCAGACAACTACAA
CTAACAGAAGAGATTTTTATTTCCTGACACACTTGTACAGTCTCTGGTAGTAAACAAAATCCAAGTCCAAGTCACCGGCCCCTAACTGTAACCT
AGTTCTTAGAAGAATTGTGCAAATCAGTAGCCAAATCATGTGGGATGGAATTCAAGATAACCCTGCCTTATACCAAACTCTACATTATGGAAAA
TAGTGTTAGAGCAAGATTCATTTTCAGTCCTTAGAGTTACCTGAAGTCCCTTCCCAGTCTACCCAAATGTTTCTGCCCTAATCTGGTTAAACTC
AAAGAGCATCTGCCAGATGAGTTAAAAATTGTCAGGTGAGTCTGACGGGGGCATCATGCTAGTTTTATAAAGCCTTTCTTTAAAAAATCAAGAG
CATGTTAAAAAAACAAACTACCGGCTGGGTGCAGTGGCTCACACCTGTAATTCCAGCACTTTGGCAGGCTGAGGTGGGCGGATCACAAGGTCAG
GATATCGAGACCATCCTGGCTAACACGGTGAAACCCCATCTCTGCTAAAAGTACAAAAAATTATCCGGGCGTGGTGGCGGGCGCCTGTAGTCCC
AGCCACTCGGGAGGCTGAGGCAGGAGAATGGCGTGAACCCGGGAGGCGGGGCTAGCAGTGAGCTGAGATCGCGTCACGGCACTCCAGCCTGGGC
AACAGAGCAAGACTCCGTCTCAAATTAAAAAAAAAAAAAAAGAAACTACCTGCTATTGGAGTATAATGGTTCATATTTCCAACTCTTAGGTGGA
ATGTATTGTTTTTGTGTCTGAAAGATTTGTAAATGCTATAATTTAACAACATTGCTTCATAAATTCCTTACCAGTAACTCAGGAGGTGAACCTT
CGGGCATTTTTCCAGATAAATCTGTGCTCTTTGTGTGCAAAGCATTGCTGCTTAGGTAACTAGTGATAATGCATAGGGTTCAATCAACACTTCA
GATATTTTCACTTTTCTTTTACTGGGCTTTTTCAAAATGGCATATGTTTATTATATTTCTGAAGTCAAAATACCAAATCTAAAACAAGTGATTT
TTTAAAAAAAATCTCTCAGCTGGGCGCAGTGGCTCACACCTATAACCCCAGCCCTTTCGGAGCTGAGATGGATCACTTGAGCTCAGGAGTTTGA
GACCATCCTGGCCAACATGGTGAAACCCCATCTCTACTGAAAATACAAAAATTAGCTGGGTGCGGTGGCGGGCACCTGTAATCTCACCTACTTG
CCACCCTGAGGCAGTAGAATTGCTTGAACCCGGGAGGCAGAGGTTCCACTCAGCCGACATCTTCCCACTGCCCTGCAGTCTGGGCAACAGAGCA
AGACTCTGTCTCAAAAAAAAAAAAAAAAAAACTCTCTGCTAATTATTTATTACTGTTGAAATGCTGCTTACCATGGCTGACAATTTTGAACAAA
AGTAAAGAATATGTCTACGATGTCAAAGTTAGCAATTCTTTCCCTAATTCTCCTTTTGTACTTTTGCTTATAAGACCTTAGTACATTTCATAGT
CAGACCTTCATAGACCCAGCTGTGTATCTTTCCATGGCCAGGCTGAGTGTCTACATTTTCAAAACATTTTAAAGTTAAAATTCAGTTCAAGAAT
ATTGTCTTTAGTCTACCAAAAGGGCTAAAAACATTTACTTTGGCCAAACGTGGTGGCTCACGCCTGTAATCCCAGCACTTTAGGAGGCTGAGGT
GGGAGGATCACTTAAGCTCAGGAGTTCGAGACCAGCCTGGCCAACACGGTGAAACCCCATCTCTACTAAAAATACAAAAATTAACCAAGCATGG
TAGTGTCCCCCTGTAATCCCAGCTACTCAGGAGGCTGAGACAGGAGAATCATTTGAACCCCGGAGGCCGACGTTGTAGTGAGCCAACATTGTGC
CACTGCACTCCAGCTTGGGTGGCAGAGCGAGACTGTCTCAAAAAAATAAAAATAAAAATAAATAATAAAATAAAAACATTTACCTTGGTAACTA
AGTGAATATTTACTCTCGTGCATCTAAGGGGAAAATAAAGAAAAAACGCCCAAAGCACTGATTGTTTGTTCAACAGATTGTAAACCATGGCTGT
CTGCATACAAGCTCAGCAGCTCCAAGGAGAAGCCCTTCATCAGATGCAAGCGTTATATGGCCAGCATTTTCCCATTGAGGTGCGGCATTATTTA
TCCCAGTGGATTGAAAGCCAAGCATGGTAGGTGAACAAGGATGTTTGCATCATATTTTGTGAAACACAAGAATATTGGAGTTTGTTTAGAAAGA
TGTCATGATGGCAACTTTCAAAGTTGTATTTTTAAACATTTTTAAATGTGATCCATAAAAAGGTATTTAAAATTTTTTTTCTTTTTATAGGTGA
AAGGTATTTAAGCCCATCTTAGATTGAGGGGATGGAATAACTCTTGCGATTTTTAAAACAGGATTTGTTCCCCTTTAATAGTAAATACTGTCTA
AAAGCATTAGAATATTACTGCTAAGAAAGAGCAGCCAACAAATATGTTTCGAGTGCCCATGGGATGCCTACCAGTGTCCTAAGTAGTAGAATTT
ATATGAAAAAAATTAAAGCCCAACATTGTGTTGTAATCCCAACACTTTGGGAGGCCAAGGCAGGTGTACTGCTTGAGCCCAGGAGTTCAAGGAA
GGCCTGAGCAACATAGCAAAACCCTGTCTCTGTACAAAATACAAAAATTAGCCAGGTGTGGTGGTGTGCACCTGTAGTACCAGCTACTTCGGAG
GCTGAGGTGGGAGGATCACTTGAGCCTGGGAGGCAAAGGTTGCTGTAACCCATGATCACACCACTACACTCCAGCCTAAGCAACAGAGTGAGAC
CCTGTCTCAAAACAAACAAACAAACAAAATTAAAGCCCTCATCTCCAGATAATTCAGTGTTTCACTGGACATGGTGGTGCACACCTATAATCCC
AGCTACCCAGGATGCTGAGGTGGGAGGATTGCTTGACCCCAGCAGTTTGAGGCCAGCCTAGGCAACATAGCAAGATCCTATGTCAAAAAATAAT
AATAATAATTTAGTGTTTAATGGGGCAGATAGGTGTGAATATGCATATAATTTAATTATAATTTATTTCTTATAGTTTATTAAATATAAATTAT
TTTACATTTCTTAATAAGTTTACATCTAACAAAAAACTAAAAATTAATAATTTAACAGTGGAGACATTGGTGTTTTTAATTTATATGTATTTTA
TATATACACATAAAGACTAAAAGAAAATTGAGTGGGTGTTGTGGCTCATGCCTGTAATCCCAGCACTTTGGGAGGCCAAGGCAGGCAGATCACT
GGAGGCCAGGAGTTCTGAGACAAGCCTGGCCAACATGGCAAAATCCCATCTCTACTGAAAATACAAAAAATTAGTTGGGCGTGGTGACGGGCGC
CTGTAATCCCAGCTACTCTGGAGGCTAAAGCATGAGAATCGCTTGAAGTCGGGAGGCAGAGGTTGCAGTGAGCCGAGATCGCACTACTGCACTC
CAGCCTGGGCAACAGAGTCAGACTGCCTTTAAAAAAAAAAAACAAAACTAAACGAAAATATACCCAAATGTTAACACTAATTACATTCTGGTGG
TAGATTTGATAACCATTTTAATTTTTCTTTATTTTCCAGATTACTGATTACATACTTGGGGGAGGGGAGAGTTGTTTTAAATTTCTTTTAGAAT
TAGTACAGTGCAGTCGACTAACAGCACAGGTTTTTAATTGGTATCATTATTACACACCAGGCAGCAGGATGTACACTGCTCTGCTCAAGGTCTG
GGAGGTTGCTGGTTGGTTACAGTCTGTCAGTCGATCTGACATATTCATTTGATACTTCAAGCCTCACCGTCTTAGCCATGGGTCCTCTTTGACA
GTCCCTGAGAGAACCAGCTTTGATGCTGCTATTTGCAAAGGGCCATATACCTGTCTGTAATGTTGCCTTAGAATAATCGCAATGCGTGTTAGTA
GCAAATTTCTGTTCAGAAACTAAAGATCACCAAACAAAGGCAATTTTCTAGGCAATTAAAAAAAAACTCAACTTCATGTAATTAAGTGAGGGAA
AAAAATTAGCTAAATATGAGAATGTCAGCTGTACCTCGTTTAGTGCTTTCCACTCCCTTTTCATTTTCTCTCCTGTTTTCTGATGTGGAGGATT
TAGAAAAACATGAGTAGGAAGTTGCTGTTAATTTCAGATTTCACTTAGCAGAGTTCTCATTTGGTGTTCTCACTGAATGAATTAAAAGACTTGT
GTTGATATTAAGGAAAACTTGAAATTTTTTAGCTTTGGTTTTCTTCAGAATATAAAGATTTTAATCAACAATTTTCAATTCTTTTTTTTTTTTG
AGATGGAGTCTCGCTCTGTCACCAGGCTGCAGTGGCGTGATCTCGGCTCACTGCAATCTCCGCCTCCCGGGTTCAAGCAATTCTCGTGCCTCAG
CTTCCCGAGTAGCTGGGACTGCAGGTGCCCGCCACCACACCGAGCTAATTTTTGTATTTTTAATAGAGACCGGGGTTCATCATGTTGGCCAGGC
TGGTCTCTAACTCCTGACCTTGTGATCTGCCCATTTCGGCCTCCCAAACTGCTGCCATTAAAAGCTTGACCCACCGGGCCCACCCAAATCTTTA
GTTCTTTATGTTTAAGCCATCTAAGCAAGTCTTTTTTTTTTTTAACAGAGAATTAAGGTTTCAAGTAAAGAACCAATACCTGACTTCTGTGAGT
TTTGCTCAGAGACTGGATCCTTTTAAGAGTGGGCTCCTGGCCGGGCACGGTGGCTCACGCCTGTAATCCCAGCACTTTGCGAGGCTGAGGTGGG
TGGATCACTTGAGATCAGGAGTTTGAGACCAGCCTGGCCAACATGGTAAAACCCCGTCTCTATTAAAAATACAAAAATTAGCCAGGCGTGGTGG
TGCACGCCTGTAATTCCAGCTACTTGGGAGGCTGAGGCAGGAGAATCACTTGAACCCGGGAGGCAGAGGTTGCAGTGAGCTAAGATCGTGCTAC
TGCATCCCAGCCTGGGTGACAGAGTGAGACTTCCTCTCAAAGAAAAAGAGTCGGTTTCCAGACCAAGGGCATTGTTGCCAGTTGAAACAGAGGT
GCAGGGCTGCCCTCATTCCTTCCAGCCTTTCATTTAATACGTATTCTATTTAATACATAGCGCACACATATTTAGAATCTGACTTGCCTTAACT
GCCTTCTCAAGGCGCACATTGGGGTGTACTATTTGATGGACTATGGTATCACTGCTTTTTTTGTTTTGTTTTGTTATGTTTTCGTAGAGAGAAT
CTTGCTTTGTCACCCAGGCTGGAGTGCAGTGGCGCAATCTTGGCTCACTGCAACCTCCACCTCCTGGGTTCAAGCACTTCTCCTGCCTCAGCCT
CCCAAGTAGCTGGGACTACACGCACATGCCGCCACGCCCGGATAATTTCTTTTGTGTTTTAGTAGAGATGGGGTTTCACCGTGTTGCCCAGGCT
GATCTCCAACTCCTGAGCTCACGTAATCCGCCTGCCTCGGCCTCCCAAAATGCTAGGATCACAGGCCTGACCCACCACGCCCGGCCGGTCTCAC
TGTTTTGATACACTTGTTGGTGCCTCTTTTTTGAAATTAGTTGCTATCTTCAGGGATTTAGGGATGGCAGAGCCTTTTAGCTACCACTATCTTG
TTTGGCTCCAGACCCAGTCAGCAGTTATTGAATGTTATTTCAGTCCGACAGCTGTTTGGTTTCTTCAGTGAGTAAGTAATTTGGTACTTCCCAG
TGTCAATTGTTGCCTAAGTAGTAGAAAGGAAGCAACCAACGGAAGGAAAGAAGGAAAGAAGGAAAGAAAGGAGGCAGCGAGACCATCCAACCCC
CGTCACCAGTTCAGTAGCTTGAAATCAATGCAAAGTCTGTAGCGTGGTTTTCCTTGTTTACTGGAAAAGTTGGCATTGCCTTCAGGTCCTGGGT
CTGCAACTAGTGACATGAATGTCTGACTCGTATAAATTCTTGTAGGCACCCTCTGCCTGAATGTCTCATTTCCAGGGCTTCTTAGAAATAGCAA
TAACTTGTTTCACCTGTCTCTACCTAAGAAACCCACAGATACCAAACTCCTTGAGGCTGGAAAGGACATGTAGAAGAGTCTTGGCCTTGTGCCT
CCTTTACACCCATGAGGGGTAACTATAAATAGGAAATTATCCAAGTCCTTATTTCCTTCAGAGTACACAGAACCCTTTCCTTTTCCTTCCATTT
CTCGGAGCAAATAGCATAAGCACCAAGCACATCATTGAGAGAAAGGAAGAAGGGCAGCACATTGTTAAAAATTTTTTTTTTTAAAAGAAGGAAA
TCTACTGTATAATCCAACTATTTAGTAAATTACAAATAGATTAACATAGGCATTGAGTCCATTTCTGTTGGGCTGTTTCTTAAAATTTGCAGTG
AGGATTAAAAACATCACTTTAGTTGAAGAAAAAGTAGGATGTGTTGAGGCTGTATTTATACAGGCTCACGTGCATGTATGTATACAGATTATGT
AATTTTTTTCCCCTAGGGACTCAGTAGATCTTGATAATCCACAGGAGAACATTAAGGCCACCCAGCTCCTGGAGGGCCTGGTGCAGGAGCTGCA
GAAGAAGGCAGAGCACCAGGTGGGGGAAGATGGGTTTTTACTGAAGATCAAGCTGGGGCACTATGCCACACAGCTCCAGGTGGGTGTGGGCTCT
GGGGTCACACTTAATTACCTTCCCTTCGGGGTCTTCTTCCTGTAGAGACTTTCTTTCTCTCCTTCAGTCCAGCATTGTTGAGTTACACGTGCAT
AAGGTTCTTGTTGTGTGGTTTTGGTCTTTTTTTTTTTTTTTACTTTTTATTTGAGAATAATTTCATACTTCCAGAAAGTTACAAGAATCATACA
AAGATTCCCCACATATCCCCATATTCACCTGTTCTTGACTTTTATGTTGTCATATACTCCTCGCTGTCTCTCGACACACACGTTTTTCAGTCAC
TGGAGAGTAAGTTGCATACCTTGTGTCCCTGCACCAGTAAGTACCTCTGTGCATTTTCTATTTATTTATTTATTTATTTATTTATTTATTTATT
TATTTATTTATTTATTTTTGAGACGGAGTCTTGCTCTGTCGCCCAGGCTGGAGTGCAGTGGAGCAATCTCAGCTCACTGCAACCTCTGCCTCCC
GGGTTCAAGCGATTCTTCTGCCTCAGCCTCTCAAGTAGCTGGGATTACAGGCTCCTGCCACCATGCCTGGCAGTTTTTTTTTTTTTTTGGTATT
TTTAGTGGAGACGGGGTTTTGCCATGTTGGCCACAGTGGTCTTCAACTCCTGACCTCAGGTGATCCGCCCACCTCAGCCTCCCAAACTGTTGAA
ATTATAGGCATGAGCCACTATGCCCGGTCCCTCTGTGCATTGTCTTTTTTTTTTTTTTTTTTTGAGATGGAGTTTCACTCTTGTTGCCCAGGCT
GGAGTGCAGTGGCGCGGTCTCGGCTCACCGCAACCTCCACCTCCCGGGTTCAAGTGATTCTCCTGCCTCAGCCTCCCAAGTAGCTGGGACTACA
GGTGCATGCCACCACACCCGCCTAATTTTTGTATTTTTAGTAGAGATGGGGTTTCTCCATGTTGGTCAGCCTGGTTTCGAACTCCCGACCTCAG
GTGATTCACCCTCCTCCGCCTCCCAAAGTGCTGGGATTACAGGCATGAGCCACCGCACCCAGCCCCTCTGTGCATTTTCTAAGAATGAACCTTG
TCTTGCATGGTTATAATACACTGATCAACTTCACTAAATTTAGCATTGATATAATACTTTTAGGTACTCATCTCGATTTCCATTTTGTCAACTA
ATTCAAACATGTCCTTTAGGGTATTTTTCCCCGCTGCAGTCCAGAATCCAGCCCAAGATCACATATTGCATCTAGTCATCATGTCTCTTTAATC
TCCTTTAATCTGGAACCATTTTGCAACCTTACTTTGTCTTTCATTACATTCGCATTTTAAAATAATTAATGTAGGCTCGGTGCAGTCGCTCACA
CCAGTAATCCCAGCACTTTGGGAGGCTGAGGCGGGTGAATCATCTGAGGTCAGGAGTTCGAGACCAGCCTGGCCAACATGGTGAAACCCCATCT
CTACTAAAAATACAGAAATTAACTGGGTGTGGCAGTGGACAACTGTAATCCCAGCTACTTGGGAGGCTGAGGCAGGAGAATCTCTTAAACCTGG
GAGGCAGAGGTTGCAGTGAGCCCAGACCGTGCCATTGCACTCCAGCCTGGGTGACAAGAGTGAAATTCCATCTCAAAAAATAATAATAATAATT
CATGTACCCCCTGCTTTTTAAACATAAAATGTTCCTCATTTGGGGTTTGTCTGATGTTTCCTCATGATTACGTTAACATTGCACATCTCCGAGC
AGACTTCTAAATATAGTTGGCTGTCCATGTCCATGGGTTCTGCATCCTGATTCCACCAACCGCAGCTCAGAAATATTCAGGAAAAGAAAAAAAG
TAAAAACAAATAGAAATAAAGATATGACAGAACAGCTATTTACATAGCATTTCTTTTGTATTAGATAGTATAAGCAATCTATAGGTGGTTTAAA
GTGTATGGGAGGATTGTATGCATTCTATGCAAAACGATGCCATTTTATATAAGGAACTTGAGCATCTGCAGATTTTGGTATCCAAGTGGGTCCT
GGAACCAGCCCCCACAGATGCCAAGGGACAATTGTAGCTGACTTGTCCTTCTCGGCTATCACATCCAGAGGGCGGCAGTGTCCCTCGTCCCCTC
ATTGGTGGTAGTAGTTTTGATTGCCCATTTGAGAGTGGCATCCAGTTTCTCCTCTTGCGAGGCTTTTAAAACTTATTTATTCCGTTTTTAAGAA
TGTTCCCCGGATACTTTAGGCAAATTAAGATGCAAAAAGTAAAACAGCACTTTGGGAGGCTAAGGCAGGTGGATCACCTGAGGTCAGGAGTTCG
AGACCAGCCTGGCCAACATGGTGGAACCCCATCTCTACTAAAAAGACAAAAATTAGCCAGGCGTGGTGGCGGGCCCCTGTAATCCCAGCTACTC
GGGGGGCTAAGGCAGGAGAATTACTTGAACCCAGGAGGCAGAGGTTGCAGTGGCCCGAGGTTGTGCCACTGCACTCCAGCCTGGGCAACAGAGT
GAGACTCAGTCTCAAAAAAAGAAAAAAGAAGTAAAACAATCTTACCTTTTCACTGCAATTGCATTTCCATGATGTGCCTTCTTGTTCCTGTTGG
TATTGGAGGAACAGTGCCTCCTGTGATTCAGGAGGTCCCTCAGGAGGCTCCCCATCCTGAGGCATGGTTAGGGCCCATAGTGTGGAGTGGCACA
AAGTGATGTCTGCTGTTGGATTCTTTCAGAACACGTATGACCGCTGCCCCATGGAGCTGGTCCGCTGCATCCGCCATATATTGTACAATGAACA
GACGTTGGTCCCAGAAGCCAACAATGTGAGTGTCCCATAGGGATGGGGAGGGCAGTCGGGAAGTCTTTTCTTACCCACCTCCCTCTGGACCCAC
CTTTGTCAAGGTGACTCAGGGTGCACTATTGGTGTCTCTTTAAAGAGAGCTTCATCCTAAGGGGCACAAAAGAAATAGGAGACTTGACTGAGTG
CAGCCCAGCACTTTGGGAGGCTGACGCACGCAGATTGCCTGAGCCCAGGAGTTCGAGACCACCCTGGGCAACATGATGAAACCCCCTCTCTACC
AAAAATACAAAAAAATTAGCTGGGCATGGTCGCATATGCCTGTAGTCCTGGCTGTTCGAAAACCTGAAGTGGGAAGATCCCTTGAGCCCAGGAG
GTAGAGGCTGCAGTGAGCCATGATCGCACCCCTGCACTCATGGGTGATGGAGTGAGACCCTGTCTCAAAAAAAAAAAAATGAAATAGAAGATGT
GCTCTCTGATCTTAACTGATTTATAACCTTGATGAGATTGCAACCACAAGACAATGTGAACAAGTGCAAAGTAATTCAACCTCAGCTGAATAAT
TTTTTAGTAAAGGCATGCTCATATTCACGTCAGAATACTTAGGAAAGACTTTTCCCTAGCCCCTCAATCAGGTGTCGAAGGAGTGGTCTGATCA
GGTCACTTACAGGGCTGGGTCCACAGAGACCTTGTTTATGCCTCCCCCATCACTTCACCCTGGGGTCTCCAGGATGCCTGCAAGAGGTTTGGCC
TCTTCCATGGTGGAAGCAAATGATTCCATCACGAGAATATCTTGAGTGAGGACACAGATGATGTGTGTTCACATGGCCCTGCTCCCCATGGCCT
CCCTAATGCTTAATGCCGTCCTTGCATTGGGTCAGTGAGTGGAAGACGAATCACTCCACCTCCTCATTAGTAGGAAAACCCAGGTGACACTGGA
CCAGGTTACGTTCTAGTTTCTCCAAAGGTAATTCAATGGACCTCGTTTGGAATACTCTGGTGGAGGTCAGATCTGAAATGGAAGCTTCTCCCAT
GCTTTGTGATCTGAGCCATGATCTGTCCGCTCACCTGTTTCTCACTTTTCCCACTCCACCCTGTGTCTCATGATGACGTTTGCTTTTCCTCCCG
ACTCAAAATTTACCTTTTGGGGTCTTACCCTGGTTGTAGGAGCTGGCTTTTCCTGGGGATTAAGGCCTCAGTCTTCCTCCCATTCGCACTGCCT
TGCACTGCCCCCCATATGTTCCCACGGTAGCTCTCCAGCTGGAAGCCTTGCTGATGCCATGTCCCAGAAACACCTCCAGATCAACCAGACGTTT
GAGGAGCTCCGACTGGTCACGCAGGACACAGAGAATGAGTTAAAAAAGCTGCAGCAGACTCAGGAGTACTTCATCATCCAGTACCAGGAGAGCC
TGAGGATCCAACGTCAGGCGCGGTGGAGGCACATTGTGCAACTTGAGGATTGGGCCTGGGGACTGGAAAGCAGCTCTGGTGGGGTTGGGAGAAA
GCACACTTTCCATTCGGAGGCAGCGACCTTTCTCTGAGTAGCAGGGACTTATTTGCATTGAAGACAGTGCTGAGAGGATGAGATTTAGGAAGGG
GAGATGACAGGACCCCCCGCTTAAGTGCATATCTCACTCAGGTCCCCACATCCAGGGAGCTTCTGAGCTGCCAGGGAAGACATTTCTTCTTTTG
AGAAGGAGCTCACAGTCTAAATAAATACTAGCAAACAATACTTTTCTATATAGTAGGCATATTATAAGTGGAGCTTTATTGAAATGATAAATAG
GAAATTATATAATTACATGTTTAAAAATTTTTCTGGCTGGGCACGGTGGCTCACACCTGTAATCCCAGCACTTTGGGAGGCTGAGGCAGGCGGA
TCACCTGAGGTCAGGAGTTCGAGACCAGCCTGGCCAACATGGTGAAACCCCGTCTCTACTAAAAATACAAAATTAGCTGGGCATCCCAGTGGCG
CATGCATGTAATCCCAGCTACTTGGGAGGCTGAGGCAAGAGAATCTCTTGAACCCGGGAGGCGGAGGTTGCAGGGAGCCGAGATCTCACCATTG
CACTCCAGCCTGGACAAAAAGAGCAAAATTCTGTCTCAAAAAAAAAAAAAAAAAATTCTAAGCTGGGAGTGGTTTTGCACACCTGTAATCCTGG
TGCTTTGGGAGGCCAGGGCAGGAGGATCACTTGAGGCCAGTAGTTTCAGACTAGCTTAGGCAACATAGTAAGACCCTATCTCTAAGGGGGAAAA
GAAACCTTTCTAACCAGGTACGTGTAAGTACTAAGGACCTGTATATGATTTCTAATGTTCTATGCAGTTATTCACAGAGGTCTTCCTGAAACAG
AGTACACTTAACGCCCCTTAGGTAGCCCTTATGGACAGAGCTTCCCTAGCCTGGTATAAATCCTGCTTAGTAGTTGGGGCACAGAGAGCTTCAG
TCTGTGGTGAGGCTGTGGAGCCCCACCAGCGCCCCCTTTTGACCCTGTAGCAGTTTGGCAGTTTGAGCTTTAATAGTTTCCTCTCTGCTTCCAT
GCCCAGCTCAGTCTCAGAGAAGCCTTGGCTCAACCACCCAGCCCCTCCAAACACACACACACACAGCACACACAGACAACACAGCACACACACA
CACCACACACATCACACACACAGCACACACACACACAGCACACACACCCCCCACAGCACACAGAGACACACAGTGCACACACACAGCACACACA
CACCACACACAGCACACACACAGACACACAGCACACACACACCACACCACACACACCACACCACACACAGCACACACACACCACACCACACACA
GCACACACACACCACACATAGCACACACACACACCACACATACACACCACACACACATAGACACACAGCACACACACACACACACAGCACATGC
TCCTCTAATTCCATGTATGTAGCTTCCATTTTATGTCTATAGTGATACTACTTATTTACTCTTTATCTTCTCCACTGGAATATAAATCTTTGAG
AAGGGACTATATCTTTTATCTCTGTACCCTGAGGCCAGCACAATGCCTGAAACATAACAGTTATTGAGTAAAGGTTGACAGGATGATTCTAGCC
TGTCTTCCTTTTTTATCTGAATCCATCCTAGACTGGCCCTAGAAGAAGGGCAGCTCCTTCAGAGAATGGAAACGGAAGATTTTGGGTGGCTATT
CCTACCCAAGTCCTTACACTGTCAACAGTATTTCTGATCCCGTGCCTGGCTTTTCTGTTTCCTGTGCATGGTGGCGGATTAGGTTTGGATTCAG
GTTGGGTTTGGATCTATCACGCAGTGTCTTCTCAGTCCCATTTGTCAAGTTCACTCAGCAGAAACACAGACTATTGATCTGCTTTTGCCAGGGA
AGGCTCAGCTGCCTCCAGTGGAGTAGGGAAATTGCTTCCCGTCTCCCTGGCCTCCTTCCCAAAAGCTTCTGCAAATTTCCAGGAGAGCCCTAAG
TGGGTCCCTTGCATACGAAGGCTGGGCCCAGGTGTTGGGATCACCACCACCCTCCCTGTGGCTGGGCAGGATTCCTGAGGTAGCCCTGCAGATA
CCTTGGATCTGTCCGTCCCTGGATGATCCTCTTCCTGCTCGATACCTGCAGTGAGATTGGAGCCATATTTCTGAGTTCTAGCTCTGGGCTGTTC
CTGGCTACATAGAGTTGCTTTTGTCTGTCCCAGCTTGTGCATCCCATGGGTCAGGGCTGGGGTCAGAGTGACCTGGCCCTCAGGGGCCAGCCAG
GAGCTCGACCAAGACATGCTGGTGCGCGCATCTTCTGACTCAGCTCAAGCAAGGACTCTGGGCTGTTGAGAAGGTGATGGGAAGGGATTAATAG
TCCCAGTGTGGTGGAGACGGGCTGTCGCTGGGAGGGTGGGGTGGGCCTGCGGTGGTGGTTGTAACTGTGGACATGTTTACTTTCTTAGCCTGGG
TTATTGATACACAGGTGCTCCTGTGTTCCACGTTCCCTGTGTCTTACCTATTTCATAATAATTTTTAAAAGGTAATGAGGCCTAACTTACTCCC
TGTCCCGCCATCAATGAGACTCCTCACAGATGGTGTGGAGGGAAGCCGCCTCCCTTCGGCCTCCCTTTCCTGAGAGAATCTTGGTGGCTCTGCA
TCCCCAGTGGCCCTGGACATGCTCTCCCTCTGCGGTCTGTCTGTCTGTCCGTGTTCCCTGGTTGGTTGGCTCGGTTCACAGTGACCTCACCTGG
ACAAACATCCTGCGCCCCTTCCAGCCTCTCAAGAATCTCATCCTCCCCTGAGGAGGGATTGCTGTGCTCAGGGACTAGACAGAGCCGCCGGGGC
TGGGTTTCCCTGAAGAGGCCTTGCTGCGGGAGGGATGGGGCCCAGCAGCTGCTTCTCTCTCTCACCTCTTCTCTCTGTCCTGTTCCCCGTTAAC
CAAGAAGCACGAACCTCACACAGAGCTCACCCATGTCAGGCAGCTTTCTGGCATCTTCCATGTCCTCATGTATTTAATCTTAATGCACCCCACT
GACGCAGGTGCCGACAATACTCCCGTTTTACACGTGGGGAAGCCAAGGCACAGGAAAGGAAAGCGATTTGCCCAGGTTCACCTGCAGTCAAGGG
CATGGGCACACTTACACCCCAGACAGCCTGGACCCATGCTGGGCCGCGAGAAGAGGGCTGGCAGCCCTGTGAGGCAGGCGGGCAGGAGGCAGGC
AGGCCTCCTCCCCAGCTGCCCCAGCACCCGTGTGCCCCCACACGGGACCGCTCACAGTCAGGAGGCTGGTGAGCAGGGCCCAGAGGCGCCTCCT
GAGGCGCCATTCATCTTGGCCCCCCTGGCTGTCAGGAACCCCGACTTGCTCTTGATGCAGTGTCTGAGCCTGGGAGGGTCTCGCTCCAGAACAG
GTGAGTGGGCTTCTCTAGGCCTCCTGTTGGACACTAGGAGGTGATGGTCATGGTTTCCTCTTCTCTCCTCTAGCTCAGTTTGCCCAGCTGGCCC
AGCTCACCCCCCAGGAGCGTCTGAGCCGGGAGACGGCCCTCCAGCAGAAGCAGGTGTCTCTGGAGGCCTGGTTGCAGCGTGAGGCACAGACACT
GCAGCAGTACCGCGTGGTGAGTGGGGTCCTGGGCCTCTCCTGGGCGTGGGTGCCATGAAGTCAGTCTCTGGGGACCCGAGGGAGGGCTGGGACC
AGCATGAGAGCAGAACCTGGGAGGGCAGGAGGCCTTTTTTCCTGGGGGCCTTGCGAGGCAGAGCAGCTTGGGGAGAGGGAACGGCCTGGGCCCT
GCCCTGCCATTTCCCAGCGAGGCCCTGCGGTGTTCTCTGGGAGCCCAGAAGGGGCTGCTCTCCTCCCTTCCCTCAGGAGCTGGCCGAGAAGCAC
CAGAAGACCCTGCAGCTGCTGCGGAAGCAGCAGACCATCATCCTGGATGACGAGCTGATCCAGTGGAAGCGGCGGCAGCAGCTGGCCGGGAACG
GCGGGCCCCCCGAGGGCAGCCTGGACGTGCTACAGTCCTGGTAATGGTGTGGGGCGGCCAGCGGGCAGGGCAGGAGGGGCTGGTGGGGATCCCG
GCTGCTTCTGCGTCCTGTGCTGGCTCCTGCAGCAGGCCCCCTCCATCCTGGGACAGCTCTTGTTTCTCCAGGGAGCCTGGTTCCTCAATCAGAC
TTTGGTCCCCATCCTGTGCACCTCCCCCAGGAAGGGGGCTGCTGTCCTGGGGGTGGGATGGGGCTCGGGTGTGTGGGGTGATGCTTGGGCTGTT
TGGGCCTAGTCAGGGTCCCCCCTCCTGTGTACGTCTCTAATTCTGGGAGGCAGGGAGCTCTGCTCTTCCCATGGGTGGCAAGTGTGGCGAAAGC
ACAGAGCCTTCCTCGGCCAACGGGAGCTGTGTCTTCGGGCCTGGCCTCTGTGCGGACCAGCCATTGTCCTCCTGTTGGCCTTGCCGCTCTCGTG
CAGGTGTGAGAAGTTGGCCGAGATCATCTGGCAGAACCGGCAGCAGATCCGCAGGGCTGAGCACCTCTGCCAGCAGCTGCCCATCCCCGGCCCA
GTGGAGGAGATGCTGGCCGAGGTCAACGCCACCATCACGGACATTATCTCAGCCCTGGTGACCAGGTGACTGCTGCCTGTTTGCCATGCCCAGG
AGCTTGGGGCAGCTCCTGCCTGCGTGGGGGGAGCCGCAGGTGCCTTCCAGACCAGCAGATCCACTTCCTGCCTCTCATCCCTCCCAACTCCATC
TCCAGTTGCTGTGGCCCTTGCCCAGTTTCTCCTGTGCACACTGCAAGAGATGCCCTTCGGCTGCAGCCCATGCCAGCTTCTGTCTTATCTCACT
CCCCTCTCCATCCTCTCCCTGGACTCTCACGGCTTCCCCAGGAACAGAGGCTGTGCCCGAGCTCTTCACCTCCTGAGCCCCTCGGGAGGCAGCC
CCACCCCCACCACAGACGGACTGAGAGCCCTTTCCCCTGCACCAGCCCCTCATCATGCATCTGTCCCTGTGTCCCCTGCAGCACGTTCATCATT
GAGAAGCAGCCTCCTCAGGTCCTGAAGACCCAGACCAAGTTTGCAGCCACTGTGCGCCTGCTGGTGGGCGGGAAGCTGAACGTGCACATGAACC
CCCCCCAGGTGAAGGCCACCATCATCAGTGAGCAGCAGGCCAAGTCTCTGCTCAAGAACGAGAACACCCGCAAGTAATTGTGCAGCTTCCTCTC
ATTCCCTGCTACTTGTCTCATGTCCTGGCAACAGGAAAAAAAGAATTCTGCCTCCTGTGTCAGATCCCTGTTTGCCCTTCTGGCCTCTTCTGGG
CATCAGCAAAGGAATATAAGGAAGTTCTTGGGCCTGGTGTGGTGGCTCACACCTGTAATCCAAGCACTTTAAGAGGCCGAGGTGGGCAGATCAC
CTGAGGTCAGGAGTTCAAGACCAGCCTGGCCAACATGGTGAAACCCTGTCTCTACTAAAAATACAAAAAATTAGCCGGGCGTGGTGGCAGGCAC
CTGTAATCCCAGCCACTAGAGAGCCTGAGGCAGGAGAATCCCTTGAACCTGGGAGGCAGAGATTGCAGTGAGCTGAGATTGCAGTGAGCTGAGA
TCACGCCACTGCACTCCAGCCTCGGCAACAAGAGCAAAACTCCGTCACAAAAAAAAAAAAAAAAGTTCTCTTACGAACTGTTTTCTGTGAAGAT
AGTTTGATCTTGTTTCATTTTGTAGAGATACGTCCGAGAAGATTACATGTATGTGTTATGTAGCTAACATTTCCTCTAAATGACGTACATTTTT
AGCAAACTTCTCTATTTCCTGCTACCCCAAATTATATTATTTACTCCGCATGTCCTGAGACCCTGGCATGGGTTTGGAGCTGGTCTCTCTCTGA
TTTGCAGTGATTACAGTGGCGACATCTTGAACAACTGCTGCCTCATGCAGTACCACCAAGCCACAGGCACCCTTAGTGCCCACTTCAGGAATAT
GGTGAGGGTGGAAGAAGAGGAAGAATGAATTTTTCCCTGGTCTTTGAGGGGAACAACTTATATCTTACGTTTCATCTTATGTTTCTTCTTTGTA
GTCCCTGAAACGAATTAAGAGGTCACACCGTCCTGGGGCAGAGTCGGTGACAGAAGAAAAATTTACAATCCTGTTTGAATCCCAGTTCACTGTT
GGTGGAAATGAGCTGCTTTTTCAAGTCAAGGTAAAGCCTTCCCTCAGCTCTGCGTGTGCGTGTGCGTGTGTGTGTGTGGTGTACTTTATGTTAC
AAAATATTTTTTTTTTATCACATAGCATCTTCTTCTTTTTGACTGTTGGTAAACTGAAAATGGACTGACCTCACCCTGAGAACTGTTTCACTTA
GGTTCTCTTAAGACATGAACAGAGGCCAAGCACAGTGGCTCATGCTTGTAATCCCAGCACTTTGGTAGGTTGAGGCAGTCAGGTCACTTGAGGT
CAAGAGTTCAAGACCAGCCTGGCTAACACAGTGAAACCCCATCTCTACTAAAAAGTACAAAAATTAGCCAGGCGTGGTGGCACGTGCCTATAAT
CCCAGCTACTCAGGAGAATCACTTGAACCTGGGAGGCTGAGGTTGTAATGAACCGAGATTGTGCCACTGCACTCCAGCCTGGGTGAAAGAGTGA
GACCCTGTCTCAAAAAAAAAAAAAAAAGACATGAACAAGCAAGCCCCAGTGCAGTCGCTCACATCTGTAATCCTAACACTTTGGGAGGCCAAGG
CAGGAGGATTGCTTGAGTTCAGGAGTTCCAGACCAGCCTGGAGCTGGTTGTGAGACCCCGTCTCAACAACAACAACAAATTAAAAAATAAAAAA
TAAAACATGAACAAACAATAATGAACTGAACTAGCTGGAGAGGTAGACTACAAAATGGATATGAGAAACATTACTTGGGATGAATTTGAGTAAA
GGTTGTGGTAAGTTTTAAATTTGTTATTTGTATTTTCTTCCCAGAAATATCTCCAATAGTCTTTTCTCTCTCCTGTTCTCCATGTAACTTTGAG
AAACTTTATCCTTCAGAAATATCTAAGCAACATTGCTTGTAAAATATTTAAAATTTCTAAGATTGTGCCAGTATGAAAAGTCATATTATTCCTG
GGGCCATTTAGATTCTGTGTCTGAGAGGAACATTAACTGAAACTTGCAAAATAAAAGTGTTGTTATAATAAGAAAAAAAAAAAAGAGAGAAGGA
GAAGAACTTGAGGCTCGTGGGCGCTCAGCCTTCTCTTGTGTCTTTTTAGACCCTGTCCCTGCCAGTGGTGGTGATCGTTCATGGCAGCCAGGAC
AACAATCCGACGGCCACTGTTCTCTGGGACAATGCTTTTGCAGAGCCTGTGAGTGTATTCCTCGGCCATCTGGGTCCCAAAATCAATGCCGGTG
TCATGAATCTCATTTATGTATTATCCTTTTAAAAAGCATAGGAAAAGGCCGGGTGCAGTGACTCACACTTGTAATCCCAGCACTTTGGGAGGCC
GAGGCGGTTGGATCACCTGAGGTCAGGAGTTCGAGACCAGCCTGGCCAACATAGCAAAATCCTGTTTCTATTAAAAAATACCGAAAAATTAGCT
GGGTGTGGTGGCACATGCCTGTAGTCCCAGCTACTTGGGAGGCTGAGGCACGACAATCACTTGAACCCGAGCGGCAGAGGTTGCAGTGAGCCGA
GATCGCACCACTGCACTCCAGCCTGGGTGACAGAGTGAGACTCCATCTCAATAATAAAAAAAAAGCATAGGAAAAAAGACGCTGTTTTCTTTAG
TGGGAGGCAGAATAGAAAGAATGTGGAAAGTGGAAAGAACATGGGCTTGACATCGGGCCAGGGTTTGAACTGACACTCCACCATTTCCTCACTC
CATGACCACATGGGCAGTTCACTTTACCTCTCTGAGCCTGAGTTTCCAGCAAGTGTCTGCAGCACAGGGATACTCTTGCTCTACAGCATTGTTA
GGAAGATTAAATTAGATAACATAGAAAATGCTTGCCACACAGTCAGTACTCAGTAAATATTATTTTGAATCCTTGGGTGAAACTATGAATGGGA
TTTGGGACTCCTAGGTTCACTCCAGAGGTTAGATCTCTCCAGAGGCTTACTTTAGCATAGAAGTTGCTTCATACCCTCTTCCTTTCAATAAATG
AAGATGCTGTTAAATGTCTTTTCCAAATTTTAGTTTCTGTGATTCATTCTAGGTCTCATGAAGAATGAGATACTTTGCCTCAAGGAAGATATAA
TTTGGTGGATTTATGCACCCAAACGCTATCTGAATCCCACTAGCATGCTGCTCGATGAGCATTTTAAAGATCTCATAGGCTGGGTGCGGTGGCT
CATGCCTATAATCCCAGCACTCCAGGAGGCCAAGGCAGGCAGATCCCTTGAGCTCCAGACTAGCCTAGGCAGCATAGCAAAATCCTGTCTCTAC
AAAAAATACCAAAAAATTAGCCAGGCATGGTACACCAGCCTCTAGTCCCAGCTACTTGGGGGGGGCTGAGGCGGGAGGATCGCCTGAGCCCAGG
AGGTCCAGGCTGCAGTGAGCCAAGATTGTGCCACTGTACTCCAGCCTGGGTGACAAAGCGACACCCTGTCTCAAAGAAAAAAATTTTTTTAGTA
GGCAAAGGTCATTTGTTACATATTTCCAATAGAGATTAGTCCATAAAAGCATGATAAAAGGGTTATCATTTGCGACATAACAGCATGTCCTTCT
CTAACTATATCCAACAAACCTTGATTATGTTTTGTTTTGTGGCTGTTAGAATATAGGATTTGACTGTTAGTTCAACAGCTCATTTACAAAACAG
CATCCACAGCATGGGTTACCTTCCAGTTGTGTGTAAAGAAGGTGAAATGGAACCTTCTGGCTCACGTGGAGAGGCTTAACGTTTCCCAACACTC
CTCTTTAGAGAAAAAGTATTTTACTGCAAAGGTGAGATTTTTAAGAGATTTACCATACTTCACTTACAAACTCTGAATCAGAAGCTCCTTTTTG
AAACTGTCTGTCTGCCTCTTGTCCTGATAAGCCTCCTAGATAAGATTGCCACATCTCTTGTTTCCTTCCCTGCCTGTCCACAGATCTCACTCTC
TGTGTACCTTTGGCATCTCCTGCACCATGTAATTACTCAAGTATCTTGTCTAAATAAATAAGCATTTTTAACATTCTCAGCTATGTTAATGACT
ACTTACATCTTATGCTATTTTTTTTTTTTTAATTTTTTTGAGACAGAGTCTTGCTCTGTCACCCAGGCTGGAGTACAGTGGCATTATCTCGGCT
CACTGCAGCTTCTGCCTTCTGCATTCAAGCGATTCTCCTGCCTCAGCCTCCCGAGAAGCTGGGATTACAGGTGCATCCCACCACGCCCAATTAA
TTTTTTATATTTTTAGTAGAGACCAGGTCTCACCATGTTGGCCAGGCTGGTCTCGAATTCTTGACCTCAAGTGATCCACCCACCTCGGCCTCCC
AAAGTGCTGGGATTATAGGCATGAGCCACTGCACCCAGCCGACCTTATGCTATGTTAAAAAAAAGACTTTCAGCTGGGCACGGTGGCTCACACC
TGTAATCCCAGCACTTTGGGAGGCCGAGGCCGGTGGATCATTTGAGGTCAGGAATTCGAGACCACCTTGGCCAACATGGTGAGACTCTCATCTC
TACTAAAAATACAAAAATTAGCTGCGCCTGCTGGTGCGTGCCTATGATCCCAGCTACTCGGGAGGCTCAGGCAGCAGAATTGCTTGAACCTGGG
AGGCAGAGGTTGTAGTGACCTGAGATGGTGCCACTGCACTCCAGCCTGGGCAACACAGTGAGACTCTGTCTCAAAAAAATAAAAAATAAAAAAT
AAATAATAAAGACTTGTGGCCAGGCACAGTGGTTTATGCCTGCAATCCCAGCACTTTGGGAGGCCAAGGCAAGCGGATCTCTTGAGCCCAGGAA
TTTGAGGCCAGCCTTGTCAACATAGCAAAACCCTGTCTCTACAAAAAACACACAAATTAGCCAGGCATGGTGGTGCACGCCTGTAGTCCCAGCT
GTTTGGGAGGCTGAGGTAGGATAATTACTTGAGCTAAGGAGGCAGAAGTTGCAATGAGCCAAGATTGGGCCACTGTACTCCAGCCTGGATGACA
GAGCGAGACCTTACCTAAAAAAAAAAATAATTGTGGAAATTTATTCTCCATATATATTTGCCTATGTGTCCTGTGGGTCAAATAACATAAGAAC
ATTGCAGCATTGTTAGTAATAGCTAAAGATGAGGCTGTAACACTGATGCCCATCTATGGGGGTCTGGTGAAATTTCTTTATGTGCTGATGGGAG
GTCTCCCAGATAGAGGATTAAGTCAAAAGAGGAAGATGCAGAACATGTGGATTTTGTCCTGTCATTTGTGAAGAAAAGGAAAAGGTATATGTCA
TTGCTGGCATGCATAGAATATCTTTGGTGGGATACACAAGAAACTGTGTTAGGTGCCTTTAAACAAAAGAACTGGGTTGCTGGCAGACAGGGAT
AGGGAGTGACTTCACTGTGTAGCCGTCTGTGCTTTTTGAATTTTAAATCATGTGAAAATCACCTATTGTAAAAAGTGTTTGTAACAAAATAAAA
TGGAGTACTATGGCTGAACGCGTTCAAAAGCAGTTTATTAAAAGTTAATCTTCTAACCTATTTCTCAATAAAGCCATCCAACTCAAAATTTCCA
CTCTTCCATACCAAGTTGTTTTACTTTGAGCTAGAATTCTTGGCAGAAGGAGTCGAGGGCAATGATTCCACGTGTGTACACACATGCCGACACG
TGTGCACACATACGCACACTCTCTCTGTTTGTACCTAGAGGACCAGTCTTTCAGATTTCTAAATTCCCAAGGTGTTTACAATCTAAGAGCCAGA
CCTTGGTTATGTTTTGGTTTCTCGCCCTTAGAACATAGGATTTGACTGTTAGTTCACCACCTCATTTACAAAACAGCATGGATAGCATGGGTTA
CCTTCTAGTTGTGTGTAAACAAGGTGAAACGGAACCTTCTGAATTTCACAGAACCGTGTACCCCAGCAGAGTCTGAATTTATCCTCAGGCCCTG
CAAGACAAATGTGTGTAACCACTCCCAGCCATCAGCGTTTTTCTTTTGTGGCTTGAGGGAATAAATCATGCAACTGTTTTCTCATCAGATTCTG
GCCTCTCTCCCTCTCAGGGCAGGGTGCCATTTGCCGTGCCTGACAAAGTGCTGTGGCCACAGCTGTGTGAGGCGCTCAACATGAAATTCAAGGC
CGAAGTGCAGAGCAACCGGGGCCTGACCAAGGAGAACCTCGTGTTCCTGGCGCAGAAACTGTTCAACAACAGCAGCAGCCACCTGGAGGACTAC
AGTGGCCTGTCTGTGTCCTCGTCCCAGTTCAACAGGGTGAGGAGCCCAGCTGCCAGCCGCTGTTAGATCAGTCCTTCCCAACCAGATGCTCTCA
GGAGTCTCCCTGCCCTGGGCCCTTATTCTTCATGTCTCTAATAAAATGTACCCAAAGTAATAGAACCTTCTGACTAGATGGACAACTTGAGAGT
TACCCAGGTCACGTGTAGAAAATTACATCTTTGCATATTTTAAATAAGTTGACCTTTGTCTACAGTTAATGCATTTTCTTCCTTTGTAAAGGAA
ATAGAGCAAAGCAGATGTTAACATAGCTCATGAGAAGCAAAGCTTTTGAGGGGATGCGAACTCTGGGCCTCTCTCCCCTTTACTCTCTTATGTT
CCCCATCTGTAATGCCAGATCAGGCTCCTATGGGACCCTCAACCCACAAAGAGGGATGCTGATGAAAGAAACTCCTCCTCTACCATCTGCTAAT
TCAGACGTCACTTTCATTTTATTTTTATTCATTTTTTTTTTTTGAGACACAGTTTCGCTCTTGTTGCCCAGGCTGGACTGCAATGGCACGATCT
TGGCTCACCGCAACCGCCGCCTCCTGGTTTCAAGCGATTCTCCTGCCTCAGCCTCCCGAGTAGCTGGGATTACAGGCATGCGGCACCACGCCCC
GCAATTTTGTATTTTTAGTGGAGACGGGGTTTCTCCATGTTGGTCAGGCTGGTCTCGAACTCCCGACCTCAGGTGATCTGCCCGCCTCGGCCTC
CCAAAGTGCTCGGATTACAGGCGTGAGTCCACTGTGCCCACCCTATTTATTTTTGTTTATTTATTTATTTTTGAGATGCTGTCTCGCTCTGTCA
CAAGCAGTGCAGTGGTACATTCTGGGCTCACTGCAACCTCCGTCTCCCAGATTCAAGTGATTCTCCTGTCTCAGCCTCCTGAGTAGCCGGGATT
ACAGGCACCCGCCACCATGTCCAGCCAATTTTTGTATTTTTAGTAGAGACGGGGTTTCACCATGTTGGCCAGGATGGTCTCAAACTCCTGACCT
CAAGTGATCTGCCTACCTCAGCCTCCCAAAGTGCTGGGATTACAGACGTGAGCCACCATGCCCGGCTATCACCTTGATTTGAATTTAATTTTTG
TTTCTTTTCCTACACCCCCCACCCCCAAGGTGTTTTAAATGTTTAATTTGGAGCCTAACAACGTGCATTTCTCAGAAGCTCCCAGGTGACACTG
ATGCTTCTGGTCACAGACCACACATCGAAACTAGCAAGGCACGGGTCAGCCCAGTCCCAGGTGAAAGTGTCTGTAGCAGGAGCCCACTGGGTGC
TGGTAACTGCAGTTTGGTATTTTCCCCTTCTTCCCTCTTTTCCTCTCCATGGGGCCATTAGGTAAAACTCACGTAGGCCGCGATATTTGTATGC
CTCTGACAAGCGTAGATGTTTGTTTCCCATTTAATTTTTCCAATTAAATATGTGTATAATATTCCAAGTGTTACTCTGGTGTTCTTATGTTCAC
TGTTGTATCTCCAACCACGACAATTTACCAGGACGGAATTACACTTTCTCCCAATGGTTTGACGGTGTGATGGAAGTGTTAAAAAAACATCTCA
AGCCTCATTGGAATGATGGGTGAGAATCATTATTTCTTAAGGCGCCGACAGAGTCTGAAGGTCTTCTCTCTGGCATCGTAAGTCACACTCTGCT
TCTGTCCTTTGTCCTTGGTGATCCAGGGCCATTTTGGGGTTTGTAAACAAGCAACAGGCCCATGACCTACTCATTAACAAGCCAGATGGGACCT
TCCTCCTGAGATTCAGTGACTCACAAATTCGCGGCATCACCATTGCTTGGAAGTTTCATTCTCGTGAGTCCCCCTCCCTTCTTATGTCCAAAGT
TAGCTGCTTTCGTTACAAAATTATTTTAACAAATAATTTAGTGGGTACTTACTATTTGTTGAGGTCAGTACTAATTATGGGAAACATAAATGTA
TAGCCATGACATACTCATAATAAGGCAGCCTATGAAAAATACTATTATGGGAAACACAATTGCTAATTTCGTTATACATGAAAGTATATGGCAG
CATGGTTACACATGAACATTTAACCATGACATGGCTACCTATGATACAAGAGTCCGTGCAGAATCTCAGTTGCTTCCCTCTTAAAATGTTTGCC
TTATTGTACTACTCTGCATAAAGCTTATTTCAGTGAAGAATTTGATGATTTGGAGTTCTTACAGTAGTAAAAATATTCCTTTATCCTAATTAAC
ATATGGATATTGCCATCAACCATTTCTACCCAAGATGTTAGTTCTTTTCTTTTTTTGAAACAGGGTCTCACTCTTTTGCCCGGGGTGGAGTGCA
GCGGCACAAACATGGCTCACTGCAGCCTTGACCTCCTGGCCTCAAGCAGTCCTCCTCCCTTAGCCTCCCGATTAGCTGGGACCACAGACGTGCA
CCACCACACCCATCTAATTTTTAAATTTTTTGTATGTTGCCCAGGCTGGTCTCGAACTCCTGGGCTCAAACAATCCCCCACTTTGGCTTCCCAA
AGTATTGGGATTACGGGCTTGAGCCACTGCACCAGGGCATCAGGAAAGTTAGTTCTCTCCTCTGACCCTCACAATAAGGGTAGCTTGCTGGTTC
ATTTTCTGTGAATTAAAGTTTTTAATAACTTTTGGCTGGGTGCGGTGGCTCATGCCTGTAATCCCAGCACTTTGGGAGCCTCAGGCAGGAGGAT
CTCTTGAGGCCAGGAGTTCAAGCCCAGCTTGGGCAACATAGTAAGACCCTGTCTCTAAGTAAATAAATAAATAAATAAGTTAATTAGGTGGGCA
TGCTGGTGCTCATGGTGTGCGCCTGTAGTCCCAGCTACTCTGGAGGCTGAGGTGGGATGATCACTTGACCCTGGGAGTTCAAGGTTACAGTGAG
TCATGATCACATCACTGTACTCCAGCCTGGGCAACAGAGTAAGACCCTGTCTCTTAAAAGAAAAAAAAAAAAAGGCTGGGTGCGGTGGCTCACG
CCTGTAATCCCAGCACTTTGGCAGGCCAAGGCAGGCGGATCATGAGCTCAGGAGATCGAGACCATCCTCGCCATCACAGTGAAACCCCCGTCTC
TACTAAAAACACAAAAAAATTAGCCGGGTGTAGTTGTGGGCACCTGTAGTCCCAGCTACTTCCGAGGCTGAGGCAGGAGAATGGCGTGAACCCG
GGAGGCGGAGCTTGCAGTGAGCCGAGATCGCCCCACTGCACTCCACCCTCGGCAACAGAGCAAGACTCCATCTCGAAAAAAAAAAAATTCATCA
CTTTTAATAGCTCTTATGCTTTCTCTGGTTGTGGGCATGCTTCCTAGCTTTTATATATCTTTGTATGGGAAATCAGGGATTGGGGGCCAGTAAA
AACTGTTTCTTCTACTGACCCCTAGAATACTGTTTGAACTTGAAACGTCTGAGATCATTGGATCTCTTGGGAAGTGATTTTTGTATTACCTGCT
TATCTACCCATCGCTTTTATTTTTATTTATTTATTTATTTATTTATTTTTGAGATGGGGTCTCTGTCACCCAGGCTGGAGTGCAGTGTCATGAT
CTCAGCTCACTGCAACCTCCACCTCCCAGCTTCAAGCCATTCTCCTACCTCAGCATAGTACCTGGGACTACGGATGAGTGCCACCACACTTGGC
TGATTTTTGTATTTTTAGTAGAGACAGGGTTCATCATGTTAGCCAGGCTGGTCTTGAACTCCTGACCTCAGGTGATCCACCTGCCTAGGCCTCC
AAAAGTGTGGGATTACAGGCATGAGCCAACATGCCTCGCCCCTACCCATGGCTTTTAAAACTATTTCAGGCCGCGCACGGTGGCTCACGCCTGT
AATCCCAGCACTTTGGGAGGCTGACGTGGGTGGATCATGAGGTCAGGAGATCAAGACCATCCTGGCTGACATAGGGAAACTCCATCTCTACTAA
AAATACAAAATTAGCCAGGTGTGGTGGCGCGTGCCTGTAATCCCAGCTACTTGCCAGGCTGAGGCAGGAGAATCGCTTGAACCAGGGAGTTGGA
GGTTGCAGTGAGCCAAGATCGCACCACTGCACTCCAACCTGGTAACAGAGTGAGACTCCGTCTCAAAATAAGTAAGTAAGTAAGTAAATAAATA
AAAGTATTTCATCTATAACCCATGGTTATTTTTAAATGGAGATTTCTATTGGAGCCATTATAATGGCAAGCTTGAATTTATAGTATGTTGGGGT
TTTAAGATTTCCTAATTCAGAAATCATGTTTAGAATGTGATTGTTCTGTTTATTGATCTAGAGGAAAGAATGTTTTGGAATCTGATGCCTTTTA
CCACCAGAGACTTCTCCATTCGGTCCCTAGCCGACCGCTTGGGAGACTTGAATTACCTTATCTACGTGTTTCCTGATCGGCCAAAAGATGAAGT
ATACTCCAAATACTACACACCAGTTCCCTGCGAGTCTGCTACTGGTAACAATGTTCGCATTCTGATTTGATTTTGTTGTGTGTGTGTGTGTGTG
TGTCTGTGTGTGTGTCTGTGCATACCTGCCTGTGTTATCTCTCTTCTCTGAAAACCCACACAATTACCATTCAGTGAGAGCAGTTTGAGCATTT
TCCAGGGCAGATGTAAACCTTGGGCTGGATTCTTCAGTCATACAGAAAACACATCTACCACTAACCCCAAATATCAAATTGAAGAAAACAAATT
CTTTTTAATACAAGCTTAGATCAAACAAAATTAGGTAAAATTTTGTTTTTCATGACAGAGATGGTTTTATTGCTTTCTGAGTTTAGAAGTATTA
CATATTTATTATAAAATTTTCAGAAGACACATAAAGGCATAAAAAAGAAAACAGGCTGGGTACGGTGGGTCATGCCTGTAATCCCAGAACTTTG
GGAGGCCAAGGCAGGCGGATCATTTGAGGTCAGGAGTTCCACACCAGCCTGGCCAACATGGTGAAACCCTATCTCTACAAAAAATACAAAAATT
AGGTGTGGTGGCAGGCACTTGTAATCGCAGGTACTCGGGAGGCTGAGGCATGAGAATCACTTGAACCCAGGAGGTGGAGATTGCAGTGAGCTGA
GACCACCCCACCACACTCCAGCCTGGGCAATAGGGTGAGACTCAGTCTCAAAAAAAAAAAAAAAAGGAAGGTCGGGCGTGGTGGCTCACACCTA
TAATCCCAGCACTTTGGGAGGCTGAGGCAGGCAGATCACCTGAGGTCGGGAGTTCAAGACCAGCCTGACCAACGTGGAGAAACCCCACCTCTAC
TGAAAATACAAAATTAGCCTGGCATGGTGGCGCATGCCTGTAATCCCAGCTACTCGGGAAGGCTGAGGCAGGAGAATTGCTTGAACCCGAGAGG
TGGAAGTTGTGGTGAGCTGAAATCGTGCCATTGCACTCCAGCCTCGGCAACAAGAGTGAAACTCTGTCTCAAAAACAAAAGAAAAGAAAACAAA
AAAACAGATACCACACAACATCTCTCATCTAGAGATTAACCTCTATGGATGATTTAGTGACTATTCTTGCACTCTATATATAGATATGTATTAT
ACATTGCATCACCCGTTGCTTGAACTTCCTGTGTGGAGGTTATCTAGCAGCAGAACGAAGACCATAAACAAGGAAACATATTCAGGCCGGGCGC
GGTGGCTCATGCCTGTAATCCCAGACTTTTGGGAGGCCGAGGCAGGCATATCACCTGAGGTCAGGAGTTTGAAACCAGCCTGGCCAACATGGTG
AAACCCCATCTCTACTAAAAATACAAAAAGTAGTCAGGTGTGGTGGTGGGCGCCTGTAATTCCAGCTACTTGGGAGACTGAGGCAGGAGAATCG
CTTGAACCAGGGAGGCAGGAGAATTGCTTGAACCAGGGAGGCAGAAGTTGCAGTGAGCCGAGACTATGCCACTGCATTCCAGTCTGGTGACAGA
GCGAGACTCCATCTCAAAAACAAACAAAACACAAATATATTAATTTAGTGATGGGCGCCCTGAAGACAAATAAAGCAAGGTAGAGGGATAGAGA
AGTGGCAGGGAGGAGAGTGGAGGTGTTATTTTACTAGAGTGGTCAAGGAGGGTTTCTCTGAGGACATGTTTGTTTTTTTTGTTGTTGTTTTGTT
TGTTTGTTTGTTTGAGATGGGGTCTCACTTTGTTGCCCAGGCTCGAGTACATTGCTCACTGCAGTCTCAAACTGCTAGACTCAAGGGATCCTCC
CACCTCAGCCTCCCACATAGTTGGGACTACAGGCATGCACCACCAATCCTCGTTAATTATTTTATTTTTATAGATATGGGGTCTTGCTGTGTTT
CCCAGACTGGTCTCAAATTCCTGGGCTCAAGCAATGTTCCTTCCTCAGTCCCCCAAGGTTCTGGGATTATAGGCATGAGCCACTCTACCTAGCC
AGGATGTGATGTTTGAAAAGACCTGAATGAAGTGAGGAAGCTAGCCATACAAAGATCTAGAGAAAAACATTGTATGCCAAGAGAACAAGTACAA
AAATCCTGGGTTGAGAATGGCCATGTCTGGAAAGCCTCAGGAGGCCCAGGGAGGCTGGAGCCTCTTGAGCAGGGAAGATAAGTGGTAGGAACTG
AGGTCACCACACTCCAGCCTTGGAGTTCAGGGTGAAAGGTTGGAGTCTAGACCATATCATATGTAATTTTTTGTGACTTTTTTTTTTTACTTGG
AAATATATCCTAGGTAGCTGGGCATGGTGGCTCATGCCTGTAATCCCAACACTTTGGGAGGCCAAGGCAGGTGGATCACTTGAGGTCAGGAGTT
TGAGACCAGCCTGGCCAACATGGTGAAACCCCATCTCTACTAAAAAACATACAAAAATTAGCCGGGCATGGTGGCAGGTACCTGTAATCCCACC
TACTCAGGAGGCTGAGGCAACAGAATCACTTGAACCCGGGAGGCGGAGCTTCCAGTGAGCCAAGATCACACCACTGCACTCCAGCCTGGGCAAC
AGAGCGAGACTCTGCCTCAAAAAAAAGAAATATATCCTATGCAGAGTTACAGCTCAGTAAATGTAATCTTCCAACCTCATTTTGATGACTGCAT
ACAATCCCACTGTATGGTGAGTTACATGTTGGTTATTTAAGATACTTATAATTTGGGCCTGGGCACAGTGGCTCATGCCTGTAATTCCAGCACA
TTAGAAGGCGGAGGAGGAAGGATTGCTTGAGCCTAGGAGTTTGAGACCAGCCTGGGCAACATAGTGAGACTCCATCTCTACAAAAAAGAAAAAG
TTAGCTGCGCTTGCTGGCGCATGCCTGTAGTCCCAGCTACTCAGAAGGCAGAGGCTGGAACATTGGTTGAGCCCAGGAATTTGAGGCTGCAATG
AGCTATGATTGTGCCATTGCACTGTAGCCTATGTGACAGAGTAAGACCCTGTCTCAGAAAAAAATAAAATCCTATTTCTGGCAGCATTTCTTTG
TTTTTAACTTCTTAGCCAATCTATAAAATACTCTGTACACTGTAAAGCACAAAAAACATGTTATGAAAAGGTCGCTCCTGACACTTGAAGCCCT
AGAAGAAGATTAGGGTAATAAGCAATTGGTGAACCCTGCAGCGTTTCACCCTCTTGGGCTACTTAAATGACAAGCTCCTTGTCCTATAATCATA
GTGTGATCGTCCTTGCCTAGTATAATTAACTTTCTACTGATTCCCATATTCACGTACCCAGAAACAAGACTTCATTCTCCCATGTCAGATAATC
ACATTCTTACCACCTGCTTACCTGGATTTCCATAGTCAGCCTTCTCAAAAGGTAACTGTGGTTGACCAGATCACGTGCAGGTTTATCTGCTCAT
CTGCCACATCCCAGTGGGGATTCTGTGGGAAGCAAAGGACACATCGCATTCTTGGCACCAACAGCACTCACTGGGACTAGTGACATCAAAGTAC
CCCAAATGCACAAACACCAGGCGTGAAGGACCGTGCAGGTGCTGGCAAAAGTAGCCTTCCCTTTCCTGAAAGACTAGACACTCACAGTTAGAGG
AACCTCAGACATCATCAGATCAGATCAGCGGCTCTCCAGTGCCTAGGCCTTTCCATTAGATCCCTGACGTGTATGCACCTCCTGCTTGAAAACC
TCCGGTGATGGCCAGGCACCGTGGCTCACACCTGTAATCTCAGCACTTTGGGAGGCCAAGGCGGGTGGATCACTTGAGGTCAGGGGTTCGAGAC
CAGCCTGACCAACATGGACAAACCCCATCTCTACCAAAAATATAAAAACTAGCTGGGCATGCTGGTGCGCACCTGTAATCCCAGCTACTCGGGA
GGCTCAACCATGAGAATCACTTGAACCCAGAAGCCAGAGGTTGCAGTGAGCTGAGATCGCACCACTGCACTCCAGTGTGGGCAACAGAGTAAGA
CTCTGTCAAAAAAGAAAAAAAAGAAAACCTCCAGTGACAAGGAAATCAGTCCTTCCTAAGATAAATCATTTCTTTTCCTGGCAGTTCTACTTAG
AACATTCTTCCCTGCATGACTGAACATTTGTCCTCTTATGACCTGCCCCTCCTGGTCCTGATTTTATCCCCTGGGTCACGTAGAACAAATCCTG
TCTCTCTGCTGCATCTTGGACTTTAAGCTTTTCAAGCTGTTTTTTTTTTTTTAATTACTATGCAATTAGTGCTTGACTTAGATTTGTATTGAAG
TACATAGTTTCCATGGTTTGAAGAATTTATTGAAACATCCAAGCCAATTTGGAAGATATCTTGAATTTCAGTTTTAGTGCTGACCTTCTCCTCT
ACCAGGTCTTCCTAACAGATTGAAATATTAAATGCTTGGCCAAGTGCGGTGGCTTATGCCTGTAATCCCAGCACCTTGGGGGGCCAAGGTGGGC
AGATAACTTGAGGTCAGGAGTTAGAAAACAGCCTGGGTGACATGGTGAAACCCCATCTCTACTAAAAATACAAAAATTAGCCAAAGTGTGGTGG
TGTACCTGTAATCCAGCTACTCAGGAGGCTGAGGCATCGGAATCATTTGAACCTGGGAGGCGGAGGCTGCAGTGAGCCGAGATTGCACCACCGT
ACTCCAGCCTGGATGAGAGAGTGAGACTGTTTCAAAAAACAAAACAAAAAAATATTAAGTGCTTGTGTCATATCCTTACATAAGTTAGGTGGTT
GTGTTGTGTCCTAAGAAAAGATGTGATTCATAAGGAAGATCACATTTTTGTTTCACTTGAGAACTTTCATTTACTACATTCTTCCCTTGGCAGC
TAAAGCTGTTGATGGATACGTGAAGCCACAGATCAAGCAAGTGGTCCCTGAGTAAGTGTCCAGGTGGCTGTGGCTCTCCTTCTGCCTCTTTCCT
CCTCCCCCAGACCCTGCCTCCCATCCTTATCCTGGCCCAGCTTTCCGCTCCCCCGGGGAAACTGTCTCAGCCCTGGGGAAGGACCTGGGCTTGG
TGATGCTATGCCAGTACCTGAGGCCCCACTCCTGTAGCTGGAGCCCCAGGGCCGAGTGGGGCACTTTGTCCTTCTACACCCTGAGAAAGACAAA
GATACCCCTTGGTCCCCTCCCCAAGTCAGCTGCCCTGGCCTCCCTGGCAGCTGAAGCTCTGTTCTCTTCCTTCTGCAGGTTTGTGAACGCATCT
GCAGATGCCGGGGGCGGCAGCGCCACGTACATGGACCAGGCCCCCTCCCCAGCTGTGTGTCCCCAGGCTCACTATAACATGTACCCACAGAAGT
AGGTGGCATTCTTGTGGGGGTCCACAGGGGAGGAACTGGGGGCTTGGCCCCAGGCTGGACCCCTGGAGGGCTGGGGTCAAAGGAATCCAGAGCT
CCTCTAGGTCTGAGACCACACAGCCCTGGCCCGGGCTCTGGCCTTTGGGAGGGAGAGATTTGACCAGAAGTGATTGGTGGTTTAGAGTCGGGTT
GGGCAAATGTCATCTGTTCCCCTCTCTCAATATGGAGATTTTTCTTGCTCTAAGGACTGAGTGGAGTTGTGATCTGTCTGGACAGCCGGTGTGA
GAGGTAACAGGGGAGGGTATTTCAGTCTGAGTTGCTGTACAGCCAGCTGTTTGAATGGTTGTTCTTTTCCCATGAAGCCCCAGGCTGAGGGCAT
AGGCTGGCTCAGACTGCTCCCCTGGTGGCCTGTGGGGCTTGGGGTGGTCTCTGGCAGGCTTTCCCCTTCCCTTCATCCCTCTTCTTTCCCTCCC
AAGCCCTGACTCAGTCCTTCACACCCATGGGGACTTCGATCTGGAGGACACAATGGACGTAGCGCGGCGTGTGGAGGAGCTCCTGGGCCGGCCA
ATGGACAGTCAGTGGATCCCGCACGCACAATCGTGACCCCGCGACCTCTCCATCTTCAGCTTCTTCATCTTCACCAGAGGAATCACTCTTGTGG
ATGTTTTAATTCCATGAATCGCTTCTCTTTTGAAACAATACTCATAATGTGAAGTGTTAATACTAGTTGTGACCTTAGTGTTTCTGTGCATGGT
GGCACCAGCGAAGGGAGTGCGAGTATGTCTTTGTGTGTGTGTGTGTGTGTGTGTGTGTGTGCGTGTTTGCACGTTATCGTGTTTCTCCCTCTCA
CTGTCTGAGAGTTTAGTTGTAGCAGAGGGGCCACAGACAGAAGCTGTGGTGGTTTTTACTTTGTGCAAAAAGGCAGTGAGTTTCGTGAAGCCTG
GAAGTTGGCCATGTGTCTTAAGAGTGGCTGGACTTTGACATGTGGCTGTTTGAATAAGAGAAGGACAAACGGAGGAGAAAGCACATGTGCTCCA
GTGAGTCTTCGTCACTCTGTCTGCCAAGCAATTGATATATAACCGTGATTCTCTCTGCTTTTCTTCTGAAATGTAGATAACTGCTTTTTGACAA
AGAGAGCCTTCCCTCTCCCCCACCCCTGTGTTCTTGGGTAGGAATGGGAAAAGGGGCAACCTACAAAGATTGTTGGGGCAAGGGAAGTCACAAG
CTTTCGGATGGGCGGTGGCTTTTCACAAAACATTTAGCTCATCTTATTCTCTCTTTGTCCTCTCTCCCCTCCTGCCCGCCCGCACCCTGGAATT
GCCACTCAGTTCCTCTGGGTGTGCACATATGTTTGGAGAAATACAGGAGAGAAAAGAGGGCCACGTAACTGAGAGCTTACAGTGCCAATGCCGT
TTGTGTTCTGGCCAGAGTGGAGTGCGCAGCCCTGACTCCCAGGCGCTGAGATTGTTGCCTGGTTACCCAGGAAGCTGCTGTTCCGGCTGCCCAG
CCTTTCTCTGAGCCAGCGGATGCACAGTCCGTGGCCTTCTTCAGGCTTATTGATGATGCTTTTTGCAAATGTTGAATCATGGTTCTGTTTCTAA
GTTGGATCTTTTTTGTTTTCTCCTTGCCACCCTAATTTGACATCAAAATTCTCTCTTGTGCATTGGGCCCTGGGTCATTCAAACCCAGGTCACC
TCATTCCCCTTCTCTGTTCACACCTAATGTCTTGAAGAGTAGGTAGCAGCAGTGTGGGCTGAACCTAGGCCAGCTTGCTTAGCGGGTCACCCTG
CTGTGAAGTCCTGGCAGGTGTTGGTAATGTGTGGAAATGCAGTCAGCAAGTTTGCTGGGGAGTTTGATAAAAGTATAAAACAAAACAAAAAAAG
CCTCGGTATAATTTTGTTCCACGACTTCTTCTGTACCTTTACACCACAACGAAGGAATGGGCTACAGCAGGTAGTGCAGGAAGAGGGGGGTGAG
CAGGTGTATTAAAATAGCTTACGGGTAAGGCCTAAAAGGTCACCCCTCGGCCCCCTCTCCAAAAGAAGGGCATGGGCACCCCCAGGAGAGGATG
GCCCCAAAAACCTTATTTTTATACATGAGAGTAAATAAACATATTTTTTTTACAAAAATAACTTCTGAATTTATCAGTGTTTTGCCGTTAAAAA
TATTCCTCTATAGTAAATTATTTATTGGAAGATGACTTTTTTAAAGCTGCCGTTTGCCTTGGCTTGGTTTCATACACTGATTTATTTTTCTATG
CCAGGCAGTAGAGTCTCTCTGCCTCTGAGGAGCAGGCTACCCGCATCCCACTCAGCCCCTCCCTACCCCTCAAGATTTGATGAAAATTCCAACC
ATGAGGATGGGTGCATCGGGGAAGGGTGAGAAGGAGAGCCTGCCTGCTCAGGGATCCACCCTCGTAGAGTCACTCCCTGCCCGTCTCCCAGAGA
TCCTTCACCAGCACCTGCCTCTGAGACCTCGCTCTCTGTTCCAGCAACCCTGGTTGGGGGGTCAGACTTGATACACTTTCAGGTTGGGAGTGGA
CCCACCCCAGGGCCTGCTGAGGACAGAGCAGCCAGGCCGTCCTGGCTCACTTTGCAGTTGGCACTGGGTTGGGGAGGAAGAGAGCTGATGAGTG
TGGCTTCCCTGAGCTGGGGTTTCCCTGCTTGTCCAGTTGTGAGCTGTCCTCGGTGTTACCGAGGCTGTGCCTAGAGAGTGGAGATTTTTGATGA
AAGGTGTCCTCGCTCTCTGCGTTCTATCTTCTCTCTCCTCCTTCTTCCTGCAAACCACAAGATAAAGGTAGTGGTGTGTCTCGACCCCATCAGC
CTCTCACCCACTCCCAGACACACACAAGTCCTCAAAAGTTTCAGCTCCGTGTGTGAGATGTGCAGGTTTTTTCTAGGGGGTAGGGGGAGACTAA
AATCGAATATAACTTAAAATGAAACTATACTTTTTATAATTTTTCTTTTTAAAACTTGGTGAAATTATTTCAGATACATATTTTAGTGTCAAGG
CAGATTAGTTATTTAGCCACCAAAAAAAAGTATTGTCTACAATTTGGGGCCTCAAATTTGACTCTGCCTCAAAAAAAAGAAATATATCCTATGC
AGAGTTACAGTCACAAAGTTGTGTATTTTATGTTACAATAAAGCCTTCCTCTGAAGGGATGGTATTGAGTCCTGTTTTTACTTTTTTCTGTCAC
ATGCCAAGGGAGGGCAGCTTGAACACGGAAAAGTTTCAAGTCATCAGCGTGACCCTGAAAAAGGGAACTGGTTGGGTGGTTGGGAGTTGGCTGC
CTCAGAGGGCACGTCATTAGCTCTGATGGCAGATGGGGCCTTTCATACCTTCCTGCTCATATGATGTTTTGAAAATAAGAATGATGGTCTCCTA
GCCTCCTGACTCCAGAAAGCTTGTTAGCCAGGATGGTCTCGATCTCCTGACCTCATGATCTGCCCACCTCAGCCTCCCAAAGTGCTGGGATTAC
AGGCGTGAGCCACTACGCCCCGCTGAAAGCCTGTCCTTAAGATTCAGCACATTTAGACTACCTGACCCTGACTTCAGCGTTGTGTGTGCGATGA
GAAATGGTGCCTTCCCTGCAGAGGCTCACAGTTGGAGAAAGCCAGCTACCACAACTGATTATGCTAGGATATCGCTTCCTGCTGCAATCAAGGC
ATCCCAAAGTCATTATGCAAAAGAACATGCAGGAAGGCCACTGAGCAACTCAAGCTTTTTTTTTATTTTATTTTTTTATTTTAATTGAGTAGCT
GGGATTACAGGCGCCACCACTCCCGGCTAATTTTTGCATTTTTAGTAGGGACGGGGTTTCACCATGTTGGCCAGGCTGGTCTGGACCTCCTGAC
CTCAGGTGATCCACCTGCCTTGGCCTCCCAAAATGCTGGGATTACAGGCGTGAACCACCATGCCCAGCCTGGAGCTGCCTCTTAAGGAACAAAT
CAGGGCCGGGCACGNNNNNNNNNNNNNNNNNNNNATAGGGCCCGGCACCGTCGCTCATGCCTGTAATCCCAGCATTTTGGGACGCCAAGGCAGG
TGGATCACCTGAGGTCGGGAGTTCGACACCAGTCTCGCCAACATGATCAAACCCCGTCTCTACTAAAAATACACAAAAATTAGCTGGGCGTGGT
GGCACGCGCCTGTAATCCCAGCTACTTGGGAGGCTGAGGCAGGAGAATCGCTTGAACCCGGCAGGCAGAGATTGCAGTGAGCCGAGATCATGCC
ATTGCATTCCAGCCTGGACAACAGAGCAAGACTCTGTCTCAAATTAAAAAAAAACAAAAAACCCAGCCATTCCACTTGTAGGTATCTACATAAG
AACCATGAGAACATATATGTACAGAAACACTTGTGACAAATGTTCATAACAGCATTATGCATAATAGCCAAACGGCAGAAACAACCCAAATGTC
CATCAACTGATGAATAAACAAAATGTATTATGGTATATCTGTACAATGGAATATACGGCAATAAAAAGGAATGAAGTGCTGACACACAGGACAT
GATGTTGTCACGGATGAACCTTCACAATATCATGCTAGATGAAAGAAGCCAGGCCGGGCACGGTGGCTCATGACTGGGACTCCAGCACTTTGGG
AGGCTGAGGCGAGGGATCACTTGAGTGCAGCAGTTCAAGACCAGCTTGGACAACACGGTGAAACCCCATCTCTACAAAAAAATACAAAAATTAG
CTGGATGTGGTGGTGCACCCCTGTAGTCCCACCTACTCAGGCAGCCTGAGGTGGGAGGATCACTTGAGCTGTGATCATGCTACTGCACTCCAGC
CTGGGTGACAGAGCGAGGAAAAAGAAAAGAAGCCAAGTACAAAAGACCGCATATTATATGATGCCATTTATAGGAAATGTCTAGGATAGGCAAA
TCTATGGAGACAGAAAGTAAATTTGAGGTTGCCTAGAGGCTGGGAAGGAGTTTGGAGGTAATAGTGAAAGGATACAGGGTTTGGGGGTTTTTTT
GTTTTGTTTTTTTTTTTGTTTGTTTTTCTTGAGACAGAGTCTCGCTCTGTCACCCAGACTGGAGTGCAGTGGCGCCATCTCGGCTCACTACAAC
CACTGCCTCCCAGTTTCAAGCGATTCTCCCACCTCAGCCTCCCAAGTAGCTGGGATTATAGGCGCCGGCCACCACACCCTGCTAATTTTTGTAT
TTTTAGTAGAGACAGTGTTTTGCCATGTTGGCCAGGCTGGTCTTGAACTCCTGACTTCAGGTGATCCACCTGCCTTGGCCTCCCAAAGTGCTGG
GATTATAGGTGTGAGCCACCACGCCCAGCCTGAGTTTGGGGTTTTTTATTTTATTTTATTTTATTTTATTTTATTTTATTTTATTTTATTTTAT
TTTATTTAACGAATTGGGCAGTCTCCTGAGGCACAGTAGGCTCAGAGAGACTCTGCAGGGTTTGTTTTTGAGGTAAGGAAAATGTTATTATCTG
TGGTGTTGGTTGCACATATCTCTGGATATACTAGATCTTGAATTCTACACTAAATGGGTGATTTGTATGGTGTGTGAATTATATCTAATAAAGC
TGTTACTGGGCCAGGTCTAGTGGCTTACACCTGTAATTCCAGCACTTTGGAAGGCCAAGGTAGGAGCATCGCTCAGACCGGGAGTTCCAGACCA
GTCTGGGCAACATAGTAAGACCCCCTGTCTCTACAAAAAAAAAAAAAAAATGTTTTTAATTAGCTGGGTGTGGTGGCACATGCCTGTAGTCCTA
GCCACTCAGAGGCTGGGGTAGGAGGATTGCTTCATCCCCGGAGTTCAAGGTTGCAATGAGCTGTGACCAGGCCACTGAACTCCAGCCTCGACAA
CAGAGCAAGACCCTGTCTTTAAATAAATAATAAAAGCAAATGCAAGATCTCAGCGAGAAAAAACGTGAAAGTGAGGTGAGTTAGCCAGAGTGAG
AACAGACCTGTTGAAAGAAGCTCAGCTGTGAAGGGAAGAGAGGTGCTCAGCAAGTAGTAGCTCTCTTCTCAAAGGTATCCAGAGGCAAGTAAGG
GGAGAAAGGGAGTGGTTTTAGTGTTTTGAGAGCCTAGGTCAAGAACACACCAGCCTGGGCAACATAGGGAGCTCTCTCGTCTCTACAAAAAATA
AAATTAGTTGAGCGTAGTGGTGCGTACCTGTAGTTCCAGTCCCTTGGGAGGCTGAGACAGGAGGATCTCTTGAGCCCAGGAGGTTGAGGTTGCA
GTGAGCTATCATCGCACCACTGCACTCCAGCCTGGGCAATAGATCAAGACCCTGTCTCAAAAACAAAAAACAAAAAAAGAGAAGTTTGTAATTT
TCCTTGGGATAAAGGCAACAGGCTAACACAGATGGTCACCCAGTAACCAGGTAAAGTTAAGATAAACTATATGTGACTGGCTGGGTGCAGTGGC
TCAGGCCTGTAATCCCAGCACTTTGGGAGGCCGAGGCAGGTGGATCACTTGAGGTCAGGAGTTTGAGACCAGCCTGGCCAACATGGTGACATGG
TGAAACCCCGTCTCTACTAAAAATACAAAAAATTAGCTGGGTGTGGTGATGCGTGCCTGTAATCCCAGCTACTCGGGAGGCTGAGGCAGGAGAA
TTGCTTGAATCTGGGAGGCGGAGGTTGCAGTGAGCTAAGATCGTGCCATTGCACTCCAGCCTGGGCAACAAGAATAGAACTCTGCCAAAAAAAC
AAAAAAAAAGAAGTTTGTAATTTTCCTTGGGATAAAGCCAGCAAGCTAACACAGATGTTCACCTAATAACCATGTAAAGTTAAGATAAACTATG
TGACTCGCTGGGTGTGGTGGCTCACACCTGTAATCCCAGCACTTTGGGAGGCCAAGGAGGGTAGATCACTTGAGGTCAAGAGTTTGAGACCAGC
CTGGCCAACATGGTGAAACCCCATCTCTACTGAAAATACAAAAATTAGCTGGGCGTGGTGGCACACACCTGTAATCCCAGCTGCTTGGGAGGCT
GGGGCAGCAGAATCGCTTGAACCTGGGAAGTGGAGGTTGCAGTGAGCTGAGATTGTGCCACTGCACTCCAGCCTGGGCAACAGAGCAAGACTGT
GTCTCAAAAAAAAAAAAAAAAAAAAAAGATAAACTATATGTGACAAGTCCTCTTACTTGAGGACTCGTTATTGTTTATCTTGAAAACGTGTATG
TAATGGGTTGTATGCGCTTGGCTATACAAAAAAGTAAGATTTCCTCCCTTCTTTGCAAGCTCTTAGCAGATCACCTGTCATGTACATCACATTG
TGGTTGAATGCTTATTCAATAATAAAATTGTTTTCTTTCTCAACTACCTTGAGGGAGAGGATGGGGTTTTTTTTGTTGTTGTTTTTGTTTTGTT
TGTTTTTGAGACGGAGTTTCGCTCATTGCCCAGGCTGGAGTACAATGGCGCAATCTCAGCTCACTGCAACCTCCGCCTCCCAAGTTCAAGTGAT
TCTTCTGCCTCAGCCTCCCAAGTAGCTGGGATTACAGGCATGTGCCACCACATCTGGCTAATTTTGTATTTTTAGTAGAGATGGGGTTTCACCA
TGTTGGTCAGGCTCGTGTCAAACTCCTGACCTCAGATGATCCACCCGCCTCAGCCTCCCAAAGTGTTGGGAATACAGGCGTGAGCCACCGCGCC
CAGCTGAGTTGGGGTTTTAAGAGTTCTTAAGCTTGCCCCTCTCCCAGCTCAAGTGGCTTAATGGCGTTCTTTCCCACACACACCTTTCTGTTCC
TTCACCTGCGCAAGTTTGAGGACCATGCTAGTCTGGTCCTAACTGCCCCAACTGCCAGCCTGGGCTAGCACCCGACATGAAGTCCTGTAGGCGA
CCTGCCCCTTTAAGAGCACCCCCCTCCTTCAGCTCCTCCCTCAGGCCTGGTGGCCGGATTTCCCAGTTGGGGCCCAGCCTTTTAAACCCATCGC
TCCTTCTTTCTGGCCGGATGTGTGCTGAGACCCAGAGTCACCCAGGGGTCTCCGTCACGTGCCAGGAGTAGGCAGAAGTGGGCTGTGACAGGTG
CATGTGGGCATGGACCCCACAGCCTGGGGCTCAGGAGGGACAAGGAGCTGGGACAGGCTGGATCCCGAGAGAAAGCAAAGCCACTCGGGAGTGG
GCAAGAGATATCGTTACGCAGCTGCTGTGTGCCCGACCGACCCTGTCCGAAACCCTTCCTGTACCTTCCCTTCCTCTTCCTTCTTTTCTCCCTT
TTTTTTCTTTTCTTTTTTTTTTTGACACGGTCTCACTCTGTTGCCCAAGCTGGGGTGCAGTCGCACGATCTTGGCTCACTGTAGCCTTGACATC
CTGGGCTCAAGGGATCCTCCCATCTCAGCCTCCCAAGTACCTGCGACTATGGGTGTGACACCACGCTGGGCTAGTTTTTCAATTTTTTGTACAG
ATGGAGTCTCCCTATGTTGCTCAGGCCGGTTGCAAACTCCTGGGCTCAAGTGATTCTCCTGCCTCAGCCTCCCAAAGTGCTGGGATTACAGATG
ATAGCCACCTCACCCGGCCCACCCCTACCTTCTGAAAGAGGCATTCTTATTCTTATTCCCATTTTGCAGATCAGGAAACAGAGCTCAGTGCAGC
CCACTAAATTGCTCAGGGCCCTACAGCTAACAAGCGGCAGAGGCAGGATCTGCACTCAGGTCAGTGGGACTCCAAAGCTTTGATAGTCTCTCCT
TTGCATCACCTGCCCCACAGGGTCCTGCCTTCTTCCCAATTTCATTTTTCCTCGGTTTTCCTGTATGCAGTAGAGCGTGGGAGGAGAAATGGAT
AATTGCTGCATGCCCCCATATCTCGGTTCCTTCCTCTTCCCAGGAGCTGCTTGGAGATGCTGCTGTGGCCACTGCTGCTGCTGCTGCTGCTGCT
GCCAACATTGGCCCTGCTCAGGCAGCAGCGGTCCCAGGATGCCAGGCTGTCCTGGCTTGCTGGCCTCCAGCACCGAGTGGCATGGGGGGCCCTG
GTCTGGGCACCCACCTGGCAGCGGCGGAGGCTGGAGCAGAGCACGCTCCATGTGCACCAGAGCCAGCAGCAGGCCCTGAGGTGGTGTCTACAGG
CAGCCCAGCGCCCCCACTGTTCCCTCAGAAGGAGCACAGGTGTGTTTCCCAGGGTCTGGGGAGGTGACCCAAAGAGAAAGAAACCCCACAGGAC
CGTCAGATTCCAGAGCCTGATGGCCCCCTTACCTCCTCCACTGCCAACGGAGTGGCCTGACCCAGAGACCCAGGACTGGCTTTCTTGTCCAAGA
TGAATTTCCCATGCTGGGTCCTTACATCCTCTCTACCCCAGGCCTATTCTCGGTTCCTCCCTCAAGGGTCTCACCAGTCTTGCCTGAAACTCTT
GGGAAACTCCCTTCCTGAGGCGTTTCTGTTGTCATTGGGGCTATAGACATAACCACCTTCCGGAATCATCTCCCTCTGACCAAGGCCAGCCAGA
CCCAGCAGGAAGACAGTGGAGAGCAGCCACTGCCCCCGACCTCAAACCAGGACCTTGGGGAGGCCTCTCTGCAGGTAATCTAGGATGGCCAGAG
CCCAGTCCTGGCCACAGCCCAGTCCCACCAGAGACCTCTGACTTGCAGCCAAACCCTCCAGTCATGGTGGGCAAAGAACCCCCAAATGGGGGCT
CTCGGTCTGGCTCTGACACCAGACCAACATTCTCTCTTTCCTCCTCCAGGCCACCTTGCTGGGTCTGGCAGCCCTAAACAAGGCCTACCCAGAA
GTGCTGGCTCAGGGACGCACTGCCCGTGTGACGCTTACATCCCCTTGGCCCCGACCCCTGCCTTGGCCTGGGAATACCCTGGGCCAGGTGGGCA
CCCCTGGAACCAAGGACCCTAGGGCCCTGCTGCTGGACGCACTCAGGTCCCCAGGGCTGAGGGCACTGGAGGCTGGGACGGCTGTCGAACTTCT
GGATGTTTTCTTGGGCCTGGAGACTGATGGTGAAGAGCTAGCTGGGGCGATAGCTGCCGGGAACCCTGGAGCGCCTCTCCGTGAACGGGCAGCT
GAGCTCCGGGAGGCCCTAGAGCAGGGGCCACGGGGACTGGCCCTTCGGCTCTGGCCAAAGCTGCAGGTGGTGGTGACTCTGGATGCAGGAGGCC
AGGCCGAGGCTGTGGCTGCCCTCGGGGCCTTGTGCTGCCAAGGACTAGCCTTCTTCTCTCCTGCTTATGCTGCCTCCGGAGGTCAGTGGGACTC
AAGGGCAGTGAGGTGGGGGCAACGGTCAGTGGTCACATGACAAGCAGCCATGCATTGGGCCCTACATAATCATCTCATTTAATCCTCCCCAACA
AGCTGCATGCTAGGGCCAGCAGCCTCCTTTTACAAGTGAAGAAATTCAGGCTCTCTAGGTTAAGAGTCTTGTAGCTGGGTGCAGTGGCTCACGC
CTGTAATCCCAGCACTTTGGGAGGCCAAGGTGGATGGATCGTTGACGTCAGCAGTTCCAGACCAGCCTGGCCAACATGGCGAAACCCCATCTCT
ACTAAAAATACAAAAACTAGCGGAGTATGGTGGTGCACGCCTATAATCCCAGCTACCTGGGAGGCTGAGGCGGGAGGATTGCTTAAACCTTGGA
GCCAGAAATTATAGTGAGCTGAGATCGCGCCATTGCACTCCAGCCTGGGTGACAGAGCAAGACCCTGTCTCAAAAAAAAAAAAAAAAAAAAAAG
GCCGGGGCAGTGGCTCACGGGTGTAATCCCAGCACTTTGGGAGAGGCTGAGGTGGGTCGATCACCTGAAGTTAGGAGTTTAAGACCAGCCTGGC
CAATGTGGTGAAACCCTGTCTCTACTAAAAATACAAAAATTAGCCAGGTGTGGTGGCAGGCGCCTGTAATCTCAGCTACTCAGGAGGCTCAGGC
AGGAGAATCACTTGAACCCACCAATCACTGAATCACTGAGCCGAGATCGCACCACTGCACTC
HUMAN SEQUENCE - mRNA
GGAGCCGTCACCCCGGGCGGCGACCCAGCGCAGGCAACTCCGCGCGGCGCCCGGCCGAGGGAGGGAGCGAGCGGGCGGGCGGGCAAGCCAGACA
GCTGGGCCGGAGCAGCCGCCGGCGCCCGAGGGGCCGAGCGAGATTGTAAACCATGGCTGTGTGGATACAAGCTCAGCAGCTCCAAGGAGAAGCC
CTTCATCAGATGCAAGCGTTATATGGCCAGCATTTTCCCATTGAGGTGCGGCATTATTTATCCCAGTGGATTGAAAGCCAAGCATGGCACTCAG
TAGATCTTGATAATCCACAGGAGAACATTAAGGCCACCCAGCTCCTGGAGGGCCTGGTGCAGGAGCTGCAGAAGAAGGCAGAGCACCAGGTGGG
GGAAGATGGGTTTTTACTGAAGATCAAGCTGGGGCACTATGCCACACAGCTCCAGAACACGTATGACCGCTGCCCCATGGAGCTGGTCCGCTGC
ATCCGCCATATATTGTACAATGAACACAGGTTGGTCCGAGAAGCCAACAATGGTAGCTCTCCAGCTGGAAGCCTTGCTGATGCCATGTCCCAGA
AACACCTCCAGATCAACCAGACGTTTGAGGAGCTGCGACTGGTCACGCAGGACACAGAGAATGAGTTAAAAAAGCTGCAGCAGACTCAGGAGTA
CTTCATCATCCAGTACCAGGAGAGCCTGAGGATCCAAGCTCAGTTTGGCCCCCTCGCCCAGCTGAGCCCCCAGGAGCGTCTGAGCCGGGAGACG
GCCCTCCAGCAGAAGCAGGTGTCTCTGGAGGCCTGGTTGCAGCGTGAGGCACAGACACTGCAGCAGTACCGCGTGGAGCTGCCCGAGAAGCACC
AGAAGACCCTGCAGCTGCTGCGGAAGCAGCAGACCATCATCCTGGATGACGAGCTGATCCAGTGGAAGCGGCGGCAGCAGCTGGCCGGGAACGG
CGGGCCCCCCGAGGGCAGCCTGGACGTGCTACAGTCCTGGTGTGAGAAGTTGGCGGAGATCATCTGGCAGAACCGGCAGCAGATCCGCAGGGCT
CAGCACCTCTGCCAGCAGCTGCCCATCCCCGGCCCAGTGGACGAGATCCTGGCCGAGGTCAACGCCACCATCACGGACATTATCTCAGCCCTGG
TGACCAGCACGTTCATCATTGAGAAGCAGCCTCCTCAGGTCCTGAAGACCCAGACCAAGTTTGCAGCCACTGTGCGCCTGCTGGTGGGCGGGAA
GCTGAACGTGCACATGAACCCCCCCCAGGTCAAGGCCACCATCATCAGTGAGCAGCAGGCCAAGTCTCTGCTCAAGAACGAGAACACCCGCAAT
GATTACAGTGGCGAGATCTTGAACAACTGCTGCGTCATGGAGTACCACCAAGCCACAGGCACCCTTAGTGCCCACTTCAGGAATATGTCCCTGA
AACGAATTAACAGGTCAGACCGTCGTGGGGCAGAGTCGGTGACAGAACAAAAATTTACAATCCTGTTTGAATCCCAGTTCAGTGTTGGTGGAAA
TGAGCTGGTTTTTCAAGTCAAGACCCTGTCCCTGCCAGTGGTGGTGATCGTTCATGGCAGCCAGGACAACAATGCGACGGCCACTGTTCTCTGG
GACAATGCTTTTGCAGAGCCTGGCAGGGTGCCATTTGCCGTGCCTGACAAAGTGCTGTGGCCACAGCTGTGTGAGGCGCTCAACATGAAATTCA
AGGCCGAAGTGCAGAGCAACCGGCGCCTGACCAAGGAGAACCTCGTGTTCCTGGCGCAGAAACTGTTCAACAACAGCAGCAGCCACCTGGAGGA
CTACAGTGGCCTGTCTGTGTCCTGGTCCCAGTTCAACAGGGAGAATTTACCAGGACGGAATTACACTTTCTGGCAATGGTTTGACGGTGTGATG
GAAGTGTTAAAAAAACATCTCAAGCCTCATTGGAATGATGGGGCCATTTTGGGGTTTGTAAACAAGCAACAGGCCCATGACCTACTGATTAACA
AGCCAGATGGGACCTTCCTCCTGAGATTCAGTGACTCACAAATTCGCGGCATCACCATTGCTTGGAAGTTTGATTCTCAGGAAAGAATGTTTTG
GAATCTGATGCCTTTTACCACCAGAGACTTCTCCATCAGGTCCCTAGCCGACCGCTTGGGAGACTTGAATTACCTTATCTACGTGTTTCCTGAT
CGGCCAAAAGATGAAGTATACTCCAAATACTACACACCAGTTCCCTGCGAGTCTGCTACTGCTAAAGCTGTTGATGGATACGTGAAGCCACAGA
TCAAGCAAGTGGTCCCTGAGTTTGTGAACGCATCTGCAGATGCCGGGGGCGGCAGCGCCACGTACATGGACCAGGCCCCCTCCCCAGCTGTGTG
TCCCCAGGCTCACTATAACATGTACCCACAGAACCCTGACTCAGTCCTTGACACCGATGGGGACTTCGATCTGGAGGACACAATGGACGTAGCG
CGGCGTGTGGAGGAGCTCCTGGGCCGGCCAATGGACAGTCAGTGGATCCCGCACGCACAATCGTGACCCCGCGACCTCTCCATCTTCAGCTTCT
TCATCTTCACCAGAGGAATCACTCTTGTGGATGTTTTAATTCCATGAATCGCTTCTCTTTTGAAACAATACTCATAATGTGAAGTGTTAATACT
AGTTGTGACCTTAGTGTTTCTGTGCATGGTGGCACCAGCGAAGGGAGTGCGAGTATGTGTTTGTGTGTGTCTGTGTGTGTGTGTGTGTGTGCGT
TGGTGCACGTTATGGTGTTTCTCCCTCTCACTGTCTGAGAGTTTAGTTGTAGCAGA
HUMAN SEQUENCE - CODING
ATGGCTGTGTGGATACAAGCTCAGCAGCTCCAAGGAGAAGCCCTTCATCAGATGCAAGCGTTATATGGCCAGCATTTTCCCATTGAGGTGCGGC
ATTATTTATCCCAGTGGATTGAAAGCCAAGCATGGGACTCAGTAGATCTTGATAATCCACAGGAGAACATTAAGGCCACCCAGCTCCTGGAGGG
CCTGGTGCAGGAGCTGCAGAAGAAGGCAGAGCACCAGGTGGGGGAAGATGGGTTTTTACTGAAGATCAAGCTGGGGCACTATGCCACACAGCTC
CAGAACACGTATGACCGCTGCCCCATGGAGCTGGTCCGCTGCATCCGCCATATATTGTACAATGAACAGAGGTTGGTCCGAGAAGCCAACAATG
GTAGCTCTCCAGCTGGAAGCCTTGCTGATGCCATGTCCCAGAAACACCTCCAGATCAACCAGACGTTTGAGGAGCTGCGACTGGTCACGCAGGA
CACAGAGAATGAGTTAAAAAAGCTGCAGCAGACTCAGGAGTACTTCATCATCCAGTACCAGGAGAGCCTGAGGATCCAAGCTCAGTTTGGCCCG
CTGGCCCAGCTGAGCCCCCAGGAGCGTCTGAGCCGGGAGACGGCCCTCCAGCAGAAGCAGGTGTCTCTGGAGGCCTGGTTGCAGCGTGAGGCAC
AGACACTGCAGCAGTACCGCGTGGAGCTGCCCGAGAAGCACCAGAAGACCCTGCAGCTGCTGCGGAAGCAGCAGACCATCATCCTGGATGACGA
GCTGATCCAGTGGAAGCGCCGGCAGCAGCTGGCCGGGAACGGCGGGCCCCCCGAGGGCAGCCTGGACGTGCTACAGTCCTGGTGTGAGAAGTTG
GCGGAGATCATCTGGCAGAACCGGCAGCAGATCCGCAGGGCTGAGCACCTCTGCCAGCAGCTGCCCATCCCCGGCCCAGTGGAGGAGATGCTGG
CCGAGGTCAACGCCACCATCACGGACATTATCTCAGCCCTGGTGACCAGCACGTTCATCATTGAGAAGCAGCCTCCTCAGGTCCTGAAGACCCA
GACCAAGTTTGCAGCCACTGTGCGCCTGCTGGTGGGCGGGAAGCTGAACGTGCACATGAACCCCCCCCAGGTGAAGGCCACCATCATCAGTGAG
CAGCAGGCCAAGTCTCTGCTCAAGAACGAGAACACCCGCAATGATTACAGTGGCGAGATCTTGAACAACTGCTGCGTCATGGAGTACCACCAAG
CCACAGGCACCCTTAGTGCCCACTTCAGGAATATGTCCCTGAAACGAATTAAGAGGTCAGACCGTCGTGGGGCAGAGTCGGTGACAGAAGAAAA
ATTTACAATCCTGTTTGAATCCCAGTTCAGTGTTGGTGGAAATGAGCTGGTTTTTCAAGTCAAGACCCTGTCCCTGCCAGTGGTGGTGATCGTT
CATGGCAGCCAGGACAACAATGCGACGGCCACTGTTCTCTGGGACAATGCTTTTGCAGAGCCTGGCAGGGTGCCATTTGCCGTGCCTGACAAAG
TGCTGTGGCCACAGCTGTGTGAGGCGCTCAACATGAAATTCAAGGCCGAAGTGCAGAGCAACCGGGGCCTGACCAAGGAGAACCTCGTGTTCCT
GGCGCAGAAACTGTTCAACAACAGCAGCAGCCACCTGGAGGACTACAGTGGCCTCTCTGTGTCCTGGTCCCAGTTCAACAGGGAGAATTTACCA
GGACGGAATTACACTTTCTGGCAATGGTTTGACGGTGTGATGGAAGTGTTAAAAAAACATCTCAAGCCTCATTGGAATGATGGGGCCATTTTGG
GGTTTGTAAACAAGCAACAGGCCCATGACCTACTGATTAACAAGCCAGATGGGACCTTCCTCCTGAGATTCAGTGACTCAGAAATTGGCCGCAT
CACCATTGCTTGGAAGTTTGATTCTCAGGAAAGAATGTTTTGGAATCTGATGCCTTTTACCACCAGAGACTTCTCCATCAGGTCCCTAGCCGAC
CGCTTGGGAGACTTGAATTACCTTATCTACGTGTTTCCTGATCGGCCAAAAGATGAAGTATACTCCAAATACTACACACCAGTTCCCTGCGAGT
CTGCTACTGCTAAAGCTGTTCATGGATACGTGAAGCCACAGATCAAGCAAGTGGTCCCTGAGTTTGTGAACGCATCTGCAGATGCCGGGGGCGG
CAGCGCCACGTACATGGACCAGGCCCCCTCCCCAGCTGTGTGTCCCCAGGCTCACTATAACATGTACCCACAGAACCCTGACTCAGTCCTTGAC
ACCGATGGGGACTTCGATCTGGAGGACACAATGGACGTAGCGCGGCCTGTGGAGGAGCTCCTGGGCCGGCCAATGGACAGTCAGTGGATCCCGC
ACGCACAATCGTGA

TABLE 28

MOUSE NOMENCLATURE
ICSGNM Sema4d
Celera mCG1273
HUMAN NOMENCLATURE
HGNC SEMA4D
Celera hCG29917
MOUSE SEQUENCE—GENOMIC
CCCACCCACCTCCTTTCACCATCATTCCGGGGCTCAGGACTCCACACAGAAATTTAGAGTGTCCTAGTAGTTCTCACGGTAGCAGCTGAAGGTT
TCTTACCCAGGGCCAGAGGGCTGCCATCTTATCCCTGGCCAGCCGCTCTCAGTACAGGTCTGCGCGGTCCGCGGGATCCAGGACTAATACCCAG
GGTCCTGGAGGAGAGTAAGAACTAGTTGGGGGCCTGTGGGGCTGGAGGAGGAGAGAATGACTCACAGGTCGCCAACCTATACAAAGAAATGGTG
TTGTCAGGATTTAAGCAGCATCATAACAATGACATGCCTTTGCCCAGGTCTCTGCAGCTGGCCGGCCACTGGTCCTCCACGGAACCCTGCTTCA
TGTCTGGCTCTCCCTGCCTCCTTAGCGCACACCCTGTTTCCGTTACTGTTCCATTGGCACCATGGCGGTCACTCTCCGTACCTGGCCACCCAGC
GTGACACCTACCCAATGCTCAGGTTTAGGGCATGAGCTGACTGAGCTAAGGCCATCTGAGGCTTGCCTGCCCCTATGAGGTAGCTTGAGATTGA
ATGCTGACCTAGATGGAGATGTCCAGCTGCTCCTGCTAGCTAGCTGGGCTTTGCCAATGTAACTACAGTAGGGCACGTCCCTGCCAACTGGAAA
TCTTGGCTCTGGGGATACTCTGAGGCCTGGGAACACCAGAGCTGGGACAACAGCTTTGTGTCAGGCAGCCACTCCGGAGCTCCTGGACCCCTCC
TGGGACTCTCCAGGGACAGGTCCTCTGGAATTCAGCAGTCAGGCTGGTGACAGTATTACCTGGGCCAGCATCGTAGAGGTGGGATACCTGGCTA
GTGGCTGGCAGGGTGAGGTTGGAAGGCCACACCCGGCTGGGAGGGAAGGTGTGGCTGCACTGTTTTCTGGAACCATAGCCACCAGAGTTTCACC
TCCTGTCTGAGCAAACGAGCAGCTAGGAATAGACAGAGCAGGCTCCTGTGGCCTGTGTCTTCAGGGGTGTGGAGTCCAGGGAACAATCTGCCGT
ACCACAACCACTCAAGATCCCCAGGTTGTGTTTGCTGTGGTGCTCAGGAGGGTGCCTGAAAGTCTATCTTGTCCCCACAGGAGCACCTAATGGT
ACCCTCTTTGTGATCTATTCCCGTTATTAGATCCCAGTGAGGAACCGTAAGTCTAAGACACAGTACGTGTATACACACACACACAGAGAGAGAG
AGAGAGAGAGAGAGAGAGAGAGAGAGGTGCATACCTAAGTGCATATCTGCAGATGTACTCACAAACATGTAGTCATGCATGTTTACACACATCT
GCAGATACATAGGTATGTGTGCACATGTGTATGCATGCACTTACATTGTTAGATGTCATGTTACATGTCATATGCATGTTATACACAGAGATAA
CATTTAACTTGGCACTTGCATGTGTGCCCATGTACATATACATAACAGACATGGCATCTGTGCACACACATGGGCATGGGTATGAGCACAGGTA
AACACATGTCAACCAGCAACACATGTCGCTGGTTGACATGCTGACACAGATGTACACAAGTTCATGTGCACAATGTGTGTGTGTCCACTCATAC
ACACACACAGTACCCTCAAGGCCATTAGCATTCTCAGGCCAGAGCAAGGTGACCATGCCCTGGCTGGGGTCGGTCCTGTCTCCAGTTGCAGCCC
CTGCTGAGATTTCCAGGGTTTGCTGGAGGCAGAGAGTTGCCCTCTGACAGCCCAGGCAGACTGCAGAGGGGGCTGCAGTTCTTTCTCCTGGGTT
TAGAGTAGGTGATCAGCCCTCTTAGTGTAGTAATGGGGCCTTCATGACCCTGTCTGTAATGGGATGTCAGGCCTTCTTCTGGAGGGCCTCAGTT
GTCGCAGGGGAGATCTCAGCTTTCCCTGGGGGAAGGGCGATGTATCTCTTTGTTTAAATTTAAACACCGAGAGCAAGGTCCTGAACTTGGCATT
GGATGGAGCCGTTCCTGGAAGGCACTGTGGCAGCCTGTCTGTGTGGGTGTGGCAGGCACATTGTTTCAAGTGAGTTTTAAATTACACCAAACAG
TGAGTAGTGTAGAGAAAAGAAGAAAAGTCAAGCCCAACGCCTTCCTCCCCTTCCTCTACTTCTCTGCTCCCCCCCTCCCCTCCTCCCCCTCCTC
TACTTCTCTGCTCCCTCCCCTCCCCTCCTCCTGTTCCCAGGGGCTTCTTTTAAAGAGCACCAGGGTGTTGGCAAGGCTTTTGCCCTCTAGTGTC
TGCCCATGGAATAACTGTGCTCTGCATTGTGGGGTTGGGTCTGCAGGGCAGGAGCCTGCAGCTGTAGTGAGCACAGCCTGGCTGAACTTGCTCT
GTGCTTCTGTTAAGGCAGCCCAGGGCCCCAGTAAACCCCACAGTTCCTACTTTTGGTCAAGACTGGCAGAGGAGTTTGCTGGCTGCCACTGAAG
GCCAGAAGGTGGCATTGGATTCCCCTGGAACTGAAGTCCCAGATGGTTGTGAGCTGCCGTGTGGGTGCTGTGAGTGGAAGCCAGGTTTTCTCAA
TCTACAAGAGCAACAACTGCTCTTAACCTCTGAGCCATCTCCCCAGCCCCTACCAATGCGTTTTATACAGCTGACATCCAAGGGCAGCCCCAGC
CAGACCACCCTGGGCTGGTGAAGAGTTTTGGTTATTTCAGCCACTTAAAAAAAAATGTTTCTCTTAAAATTTGTTTTAGTCTTGTGTCCTGACT
TTTTCACTCCAGGCACAAGCTGAGGCTCTTGCTCTCTGCCCCTTGTCATGCACCTCCACCCCTCCCCCACCCTCCTAATAAATGTATTCCCCAC
CCCCCACCCCCTTTGGTGACTGAGGCTGCACTGTGGGCCCTGGTAGCACATTCTGATTATATTTCCACTAAGGACCATTCTCTCTTCCCCTGGG
GTTCTAATTGCTCCATTTTGAGTGTTTGGTAATTCATTCTTAGCTGTTGGTGCAGCATCTTAGCCATGTTATCAGGCCCCAGCCTCTGAGACTT
TCCTTTTTGCAGCTGATAGAATGAGAAAAAAAAAAAAAACCCTTATTATTCTATGTTCACAGAATGGCTGTTAGACCTCCAGACATCTAGTCTT
GAGTTTCAGATAGCAGCATCAAGAGCGAGCTTAGGGGTCCAAATGCTGGTGGTTTTTTTTTTTTTTTTTTTTATTATTGTTGTTAGCCTATTTC
ATGGAATTGTTTCTAGTGCTTTCCACTTAGATTTCTTTAACTAGAAGCAGGGTAAGTTACAAAGGTGTTGAACTGGGCGTTCTGCCCACCAGTG
AGAGCTCAGGATCTGTTACCTAGAAGGAGGCAGGAAGTCACAGAAGCAGGAGGAGGCCAGGCTGGGTGGCAGGAGATTTGAGGGGAGGGGAGAG
CTACTGAGCGTGGGGAGGGGCAGTTGGATGAGGAAGGAGGTGCATCCAAGACAAGTTTGCAGATTGGGTGTCCTTACTAGCCATGGACCCAGCC
AGAGAGTGGGGCTGCAGGTAGGTGAGTGTGCTTTAGTGGCCAGTGCCTTCCTTGTCCTCCCGTCCACGTGGTTGACTAAGGAGCTGAGGGAGTA
GTAGGGTTTCATCCTGGGTCTCGGGGATACATGTGTCTTGTGACACTCTCACAGCTCTGAGAAGGAAGACGTTAGGTTTCTAAAATCCTGTCCT
GATGCTCGTGAACTTTAGGGGCAGCCCTTCTGCACCCTGCCCGGCATGTCCTTTCATGTTTCTCAGCCAGATGGGTCTCACTCCTTGTGGTGTC
TATGAACCACCTCCTGTCCTTCCGTGCTGTGGTGGGGCATCCTAATTAGCGGCTTGCGTGTCCATCAGCTCACAGGTTCAGGGCACCATGTTGG
GTCTGGTGTTGGGCAAAGCCCTTCTGGTCTGTTAGCCCCTGGTTCTGGACTCTCTTTCTTGGTATCTTGCTCGGCCCCACCCCACTTCTTTTCC
AGGGCTTAATTAATTAGCTTGTTGTTAATCAATTTTTGTTAATTAACAAAATAATTAATTAAATCCTGAGCCAGGACCTCACTGTGGAACCACG
GTAGGACTGGGACTCACTGTGTAGACTGGAGCTTGCCTCCCCCACCCCACCCCACCCCCCCCAGTGTTGCGATTATGGCCTGGGACTACATATA
GACTAAACTAGCAATGAACTCACAGAGCTCCCTCTGACCTATGGTTAAAGGCCTGTGCTACCACCTATTAATTTTGGAATCTGTTTTGTATCTT
TAAATTTTATTTCAGTCAGGGTCTTCTGTAGCCCAGACTGACCTCAGACTACATAGCTAAGGCTAGCCCCGAACTCCTCTTTCTTCTGCCTTCA
CCTGGTGGTGCATTGACATTCTAGGTTTATGAAGGGCTGGGGATAGAACTTTGGGGCTTTGCGCATGTGAAGCAAACATTCTACCAATAGAGAT
AAGGAATGTTTTCTTTTCTTTTTTTTTTTTTTTAAATAATCTATTTATTTATTACATGTAAGTACTGTCTTCAGACACACCAGAGGAGGGCGCC
AGATCTCATTAGGGATGGTTGTGAGCCACCATGTGGTTGCTGGGATTTGAACTCAGGACCTTCGGAAGAGCAGTCAGTGCTCTTAACCGCTGAG
CCATCTCTCCAGCCCAGGAATGTTTTCTTATAAAAAATGTTCAAGCAGGAATGTGACCTGCATGACCTTGGCTGGCGACCCTGGCAGGCGTTCA
GCCTTCTCTGTTCACACCAACATCTACTTCCATCAGGGGGATGTGAAGAGGAAAGAAATATCCATGTCCCATGCTACCTGACCCTCGCAGCTGG
AACAAATCAGAGGGCCTCCTGCGTGTCACAGCTCCCTTGTTCTGCTCATCTCCAGTTTCTGCAACACCCAGGATCTTCTGGGCTTAGACCAGTG
CCACCTGTGCCTCTGTACCTAGAGGGTGCTTGAGATCAGAGTGGTGTTGCTGTCCTGGCTTTCCTTAGCTGTGAATGCTCAGTGTTGGAGGGAG
TTGGTCACCATGGCTTGGACCCTAGCAACCCACAGCCCCCTTCCTCACTGTGGCAGGAGAATGGGCTCTTACGCGACAGGACGCAGCACTGTGA
ACTTGCTCAGCAAAACGCAGAGTTCATAATTGTCGGACTCACCACTGTCTGTTTCCCAGTCACTGCCTCAGTTCTTGGCTTCCTGCTTTCTGGT
TGGATCAGGGTAGGAGTCACCCCTTGTGCTTGGCTGCTGTGAATTTCTAGGGTAGGACAGGGGTCCGTCTGGCTCCTATTCTGTAGAACCGCTG
TCTATAATTGGAGTCACCTCTTTGCATGTCACCTGTATCTCCTTTTGAGCTATAGCGATAGTTAAGGAAAATGGTGAGCTGAACTGGCTTAGAA
TATCCCACTCCTTGGAGGAAGGAGGGGTTCGGGCTATCTTTAGGAAGCTTCTAAATCCATTGGGATAGAAACCAGACAGTCGGGGCTGGTGAGA
TGGCTCAGTGGGTAAGAGCACCCGACTGCTCTTCTGAAGGTCCAGAGTTCAAATCCCAGCAACCACATGGTGGCTCACAACCATCCGTAATGAG
ATCTGATGCCCTCTTCTGGTGTCTGAAGACAGCTACAGTGTACTTACATATAATCAATAAATAAATTATAAAAAAAAAAAAAGAGAGAGGAAGA
TAAAGAAGAGAGGGGGCACTTTCTATTTTAAAAAAAAAAAAAAAAGAAACCAGACAGTCTGTCCTTTAGTACCGGACAGACTAAGGGACTGAAT
AGAGATGCTGGAAGAGCCTGCCCTGCCCTCTGTGGGAGGAAGGGTGGGGCAGGGGAGGCCTGGCAAGTGGGTAGATGCCATGCCAGCATTGGGC
TCACATCTGGAGACTGTTGTGCTCCCTTCAGCTTCTAGTAACCACACCCAAAGGTGAGGCGGATCTGTGTATCAGGGCAGCTGGTGAGTGACTC
AGCGCTGAGCTCTGCATCCTGGGATCCTGGTGTGTGCTGGGCCTCAGGCTTGGCTAGGGCAGGTGAGGACAGGGACAGAGGCTCAAGGGACCTG
GCCACAAGTCCATCAGACAGCAAAGCCTGGTCTTCTGTTTCTTGTTTGTGGACAAATCAGAGTTAATTGCTAGTGTTTCCATGGTAATTAACGG
CTTCCTGGTGAGGCAGGAAGAGCTCGGGTTGCCTCCTGGGCTAGCTATGACTTTTGATGTGACCCACTACTCCTGCAGAATGGGGCCCTGAGGC
TATGTCTTCATGGGTACGGAGGTTAGTGTGTGCTTACATACTGTATGTATGTATGTGAGCACATGCGTGCATTTTCGTGCACATTTGTGTATGG
CTATGTGCATTCTGTGTGTCTACCCATGCATGTGCACCCTTACATGTATTCCCAGGGGAGTGCCCTGTAACCGCAAACCTCTGATGTCTGGTAA
TATTCCTGGGTTAGCATGTCAGAAGTTGGGAAGTGAGCCCTCCAGGGAAACTGAGACCCGTACTGTTCCTTTCAGTTGTGGCATGTCACCAAGC
ACCTGGCTGTCCCTCTAGCTAGTTATTTCCACCTTGGTGGGTCCATCCATCTTGGTGTGTTTTAGCCTTGTTGTCTTCTCTCTGTGGGATCTGT
CTGTCTGGCAGCCCCACAAATCCTGGATGCCTTCCATGTCCCTCAGCAGGCAGGTTCAGACAGGATAGACAGTAGAAAGGGATGGAGGGGACTG
AGAACCATCTGGAGCCGCTGGGACCTATAGTCTCGTCCTTTCCTTCTTTTGCAGGTCAGACGGGAACACCCGCAGCCTTGGCATGACGTCGTGA
AGGTGGCCATTGCTAACCTGACATGTGGGGACTCAGGAACCCCACCCCTTATGGGCTCCAGTCTGTGCTGCTGGCCCCAGCTCTGGGGCTCTAA
GAGGTCCTTGCTGCTACCCCACAGCAGCCTGCTGCCATCCATGTGTGCCCGTTGCTGAAGGCCTCGGTGGCCCCTGCCCATGAGGATGTGTGCC
CCCGTTAGGGGGCTGTTCTTGGCCCTGGTGGTAGTGTTGAGAACCGCGGGTGGCATTTGCACCTGTGCCTCGGCTCACCTGGGAACATGGAGGT
AAGTGAGTGTGAGCCATGAGTGAGTGTGAGTGTGGGTGCAGCAGCTCTGGAGAGTGTGGGGTCCCTGCTTTGGATGCTCTGTGGGGTCTGTCAG
GGCAGGCTTAAGCCGCTGGGATGTCAAAGGATGCTGTGTGACCAGGAGACCCCACAGCAAGCGCCAAGCTTAGGTTGGACGGTGTATGTGACCT
CAGTGGCTCACGGCTTACTTAATGCTGACGACATCCCAGGTACTTATGCTGCACTGTTGAGATTAGCTCAGCCTCAGAGAGGTCTCCAGGCCCG
ATTTCCAGGATGGGGACACACTGGCTGTCCCACATTCCAGGGAGGGAACTTTGTGAAGACAGAAGCCGGGAAGCAAAGTGCACTGAGAATGTCC
AACTCTGAAGCTACCTGGACACTCCGGGTGCTGACTTGTAGGCAAGTTCAGATGAAGTTCATTGATGGGGGAGGGGGCAGAATGCTGAGCTCAG
CATCACAGCAGGAACCTCCACACTCAGGAAGTTGATGCAGGAGGATCGTGAGTTCCAGGCCAGCCTGGGCCACGGAGAGAGACCCTGTCTAAGA
AGACCAGTATTGTGTATGTCCTGCCTGTGTTAACATGTTCTTAAACTGGTGCAGGTACGAAAAAGCATCACTTGTCTCTGGAAATCATTGGTAA
TGAGTGTAAATTTGTTTTGGAGAGAGATGTGTCCCCCCCACCCCCACCCCCATCATGACTTTTCTTTTTTTAAAGGGGGCACCGGGGAAAGAAC
ATAAACCCTGCACACACTAGGCAGATGTTCTACCAATGAGCATCTCAGCCCCTCGTTCCAGAGTTTTCGGTGTTGAGGGTTTCAGAGTAAGGCA
TTTACTTCACGAGTGAGAAAGTTCTCAGTCATTACGTATTTTGAGCCTACAGTGGGTGGTTAAAGACACATTAAAGAGGTAGAGTGTAAACCAA
GTGTCTTCGTCAGGGTTTCCACTGCTGAGAACAGACACCATGGCCAAGGCAACTCTTATAAAGGACACTGTTTAATTGGGGCTGGCTTACAGGT
TTAGAGATTCAGACCATTATCATCAAGGCAGGAAGCATGGCAGCATCCAGGCAGGCATGGTGCAGGAGGAGCTGAGAGTTCTACGTCTTCATCT
GAGGGCAAACAAGAAAAGACTGATTTCCAGGGAGCTGGGAATGATTCTCAAAGCCCACCTCCACAGTGACATACTTCCTCCAACAAGGACACAT
CTCCTAATAGTGCCACTCCCTGGGCCATGCATATTCAAACCACCACACCAAGTGTCTGTGATGTGCTAGAAGCCCCAGCATGAGAGGTTTGGAT
CTGGGAGACAGAAAAGGAGATCAGGAGTATAGCCTGGGGATGCATGCCATCCTCAGGGATCCAGCCGTGGGCTAGCTCCTCTCTTGTACTTGGT
GGCTGCATTGAGGTTACCTGGGGTGGGGGGGGGGNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNAACACACACACACACACACACACACACACACACACACACACACACACACAGCCTAGAAAGATAACAAACTCCCA
CAAGTTGTCCCCACACAGCCACTTACACACCATCACACATTCACATCTACACATAATAAAGACGTAATTGAGCACAAGGAGATGGCAGCAGCAT
CGTGGGGGTGGGGAGTAGGTAGGGGGAGGTGGTGAAGGGGAATTGAGGTAGCTTACACTAATCACTGGAAACGTCAAAAAAATGTAATAGAAAG
CTTTAAGGCTGATAAAAAAGAACAGATGGAAGCCCCCAGTTTTGGATAGAATTTAATTGACTATACTTTCATTATGTGGGGCTAGGGGGTCATA
GGACGACAACCTTGGGTGTCTTTCCTTGGGTGCTGGCCACTTTTAAAATTTCATTTTTTTTGAGACGTGGTCTCTCAACCAGCCTGGAGCTTGT
TAAGTAGACTGGCATGGCATCACTGCCTCCCCAGTGCACAGTGCCACACCTAGCCTTTTCACATGAATTCTGGGGATCGAATTCAGGTTTCTGT
GCTTCCTGGCAAGCACCCACTGAGCCATTCGTTGGTCTCCCCAGCCCTCTGCTGCTTTGTCCGAAGTTCAGGGCTGTGTTATCCCTGCTAGACT
GGGGATGATGGTCCCAGAACATGAGGAAGCCCTAAGGATGACTCTGGGGTCTCAGTCCCTGCCTGCTCCCTGAACCTCTTCCTAGTCTGACTTT
ATTCTGGCTGCAAACCAGTGAGCTTTGAGCTGTGGTGTGCCTTCTGTTGCTGTCTGCTTTATCCAGCAGTTCCTTGGGGTGGGGGTGGGGGGAA
CTGTGGTGAACTGGCTCTGACACGTTCCTCCCTCACAGGTATATTGGAAGGTCTCTGAAGACAAAAAATCCAAGTGTGCAGAGAAGGGGAAATC
AAAGCAGGTAATTGACCGCACGTACAAAGAGCCCACGCACACAGTTCGCAGTTATTTGGATCTTTTCCAGTAGCAGTCTTCTGCATGCCTACCT
GGTATCACGTGAGTCTGGTGGGCTCGGTCCTGCTGTCTCAACCCTACCAGCATCAGGATTGGTGCTCTCAGGCCAGTGCCCTGCATCCTCCGCA
CTGGAGAAGCCTGTTCTCACGGCTTGGCATTATGACTACAGGATGTTGTTTCTGCTGCCCACTAGTACCTGGTGTAGAATTGGCCTCCCCTATA
TGTTTCTGGCCTTCTTGAACCAGCTGAGGCCTAAGAGGTGTTGGGGATTTGATGGGATTTGTTATTGTTGGGTTTATTTTTTGTTGTTGTTGGG
TTTTGTTTTTTTGTGGGTTGTTTTTTTTTTTTTTCCTGTATTTTGTTGTCAGATACCAGTATGCGCACTCAGGGAGACTGAGCCTTGTATCCTT
TTTCTCAGGAGCTCTGAGTACCCTCTTGTGCCTGCCCACCTGGTGTCCCACCAGTGTCCCAGATGTTTTTAGATCACGAATGTTTTTAGATCAC
AGGAGCCAGGTTGTCCCCAATATTGGTCATGGTGGCTCAGCAGTGGCAGAGTTTATCAGCTGTTAGCTGATTTCAGAAACAGGGATTAAAACAA
CCACTAGGAGCCGGGCGTGGTGGTACACACCTTTAATCCCAGCACTCTGGAGGCAGAGGCAGGCGGATTTCTGATTTCGAGGCCAGCCTAGTCT
ACAGAGTGAGTTCCAGGACAGCCAGGGCTACAGAGAGAAACCCTGTCTCGGAAAACAAAAACAAAAACCAACCACTAGGTTAAACTTTCTTGCC
TAAATGGTAACTGAACACTTTCCCCGTCTTACTGGTCTCTAACTGTAGTGTTCACTCAGGACCCAAGGCAGGCAGGCATTACGATAGGCTCCCT
GCTGCAGGAAATCTGTGTAGCGGAGTGAGCTGATGCCTTGCACCACTGGATGTGGGGACCCACAGTGACCTTGGCATGGAGCCCTCCTGTGATA
CCCTGTCCATTTGAGGGTTGTTGGGGAAAAAAGCAACAACAACAACAACAACAACAAAAACCCCTTTCCCAACTTGAGATAATTTCAGGCCATA
GCCCATAGACTCTAAGTGCTGTTCTCCTGAAATAGCACATGGGCTCCAAATTGCCGTCTTTGTTTTCCCACTGGGCCCTCAGGGATACACAGCT
GGCTGTACTCTTGGGTCCAGCCTCTCTCTCAGTTCCTGCTAGTCGCTATTTCTAGTTCTGTGCTGGTGGCAAGAGTATAGGGTGGGGTCCCCAG
GAAGGTAGAAGGAGCCTGTACAATGTCTGTTTTGGCAATATGGGCCCCCAAAATGAACGGTGTGTGTTCAGCAAGGGTAAATACATTGCCATCT
GTAGTGGGCTCCCCAGTACAATTTGAGGTCAGCTCTGGTTTGGTCTTTGCTTTGCTTGGTCACCAGATACAGGCTGGCCATCTACTTAGGGATA
GAGCTAAAGTCATGCCTGGCACCTGCACTGCATGTGGACCACAGAGATAAAGTTTCCGTTGCAGAGCCTGGCTCATGGTGCAGTGTGCCCCCTC
TATGGGCATAGACAGACCAAACCTGAGCAGAGGAGAGAACAAGGCAGCAAATGACTGGCCTTTACCATGTGGATACAAGGGTTCTTTGATGGGT
GCCTGTTAATGGGGTGTTTCCCTTGCAGACGGAATGCCTAAACTACATTCGAGTACTACAGCCACTAAGCAGCACTTCCCTCTATGTGTGTGGG
ACCAATGCGTTCCAGCCCACCTGTGACCACCTGGTAAGACCACAACATATCCCTGTGTGAAGTGCTCACGTACCAGCCTGGCTAGCGACTAAAG
CAGAGCCAGCCTGTCTCAGTCAGGCTTTCTATTCCTGCACAAAACATCATGCATGACTAAGAAGCAAGCTGGGGAGGAAAGGGTTTACTCAGCT
TACACTTCCACATTGCTATTCATCACCAAGGGAAGTCAGGACTGGAACTTAAACAGGTCAGGAAGCAGGAGCTGATGTAGAGGCCATGAAGGAA
TGCTGCTTACTGGCTTGCTTCCCCTGGCTTGCTCAGTTTGCTTTCTTATAGAACCAAGACTACCATCCCAGGGATGGCACCACCCACAATGGAC
CCTCCCACCCTTGATCACTAACTGAGAAAATGCCTTACAGCTGGATCTCATGGAGGCATTTCCTCAAGGGAGGCTCCTTTCTCTGTGATAACTC
CAGCTTGTGTCAAGTTGACATGCAAAACCAGACAGTACACAGCCTCTCCTGTTCTTTCTGCAAACTGCAAGGACTAAGAACTATACAGTCGGAT
TTCTGACAGCAACAATTGCCCCGTTCCTCCTGATGAGGGTTCTGTGAGGTCCATGGGATGATCTATGGTAGCCATGAGGTAGCTGTACTTTCCA
AAAAGAATAAATACCGAGAGATCGGTGTTAGATACTGAAACATCAAATTGGATGTGCAAAACATCCAGCAGAAATCAGGGCAATCTGATTAAGG
AAGCTGGCTGGGGTTCCCAGCAGAGTTCTAGTCAGAGCAAGAATGTCCATTTGGGTGGGACTTTTAAATAAAAGAACAAACGGCAGCAGAGAGA
TGCCGTTGCCTTGGGAGCATCAGGCATCCATCAAGAACTGACTGGGGAAGTTGAGGCAGTCAGCTGGAGCCATCTCTCTGTAGCTGAGATCCCA
TGGGTGACCTCCCCCGCCATCCCCACCCTCACTCCCCAACCCCCCCACCCCGCCACTGTATTGTGAGATAAATAACAGTAAGCTCTTCTGCAGA
TGGTTCACCCCCTAGATGCTCTCAAGGGCACAGTGTTTCTCAAGGGCACAGTGTTTCTCAAGGGCACAGTGTTTTTACTCTGGGGTTGCTTGCT
CTTCTCTCTGTCACAGAGTCAGGGACCTTGGAAGACACTGGGAAAACATGCTTGGGTCTGAAATGGAGGAGGGGAAACCCTGCTGCCAGAACCA
TTTTCCCCCTTGAGCTCACACAGCACAAGAAAGCTTACCAATACGATGTATATTACAGGTCACTTACTTACTTAGCCCTTGCTGTGACCAAATA
CTTGATAAAATAAGTAGCTTAAGGGGAAAAGATTATCGAAGGCACCTCAGCCAAACAGAAGGCATCTTGTGGACAAGTAATGCTGGCCTGTAAA
CACCCAGGCGCACCCCCAGAAACCCGCTTTCACGGTAGGTCTCACTTCTACATCCACCACCTGGAGAGACACCCATGAGCCCATGGGTGACATT
TTACAAATAAAACGATAACAAATATGAAAACGTAATAGATGGGTTTCCTCTCTTCTCTGCTGGGCATAGATCCAAATTTTGCACCAAATGCCAA
AACCTTGGCTCTTCCCAAGCCTTATATTGTATACCTGATGGCCACGAGGCTAGGTGTTCAAGGAAAGGTAGCTAGCCTGTTCAAGGAAAGGTTG
GTCTGCTGAGCACGGGGTGAGTGATGCCATGCAGGGCAGGGCAGGGCAGCTCAGGAGGCAGGAGAGTAGTTCATTGCTCTGCTGCAGCCGGCTG
CAGATGACTGGAATCCCGGGTGGGGGAGGCTTCTGAAATGAAGAGCCTCATTCACCTAAACTCAGCCAGCTGAAGTTTAAAGCTTGTTCCCAGT
AAGAGTTCAGTTAGAGTTGTCTTAGTTAGGGTTGTACTGCTGTGAACAGACACCATGACCAATGCAAGGCTTACAAAGGACAACATTTAATCGG
GGCTGGCTTATAGGTTCAGAGGTTCAGTCCATTATCATTAAGGTAGGAACATGGCGGCAGCCAGGCAGTGCCTGGTGCAGGCAGAGCTGAGAGT
TCTACATCTTCATCTGAAGGCTGCTAGCAGAATACTGGCTTCCAGGCAGCTAGGATGAGGGTCTTATAGCCCATACCCACAGTGACACACCTAC
TCCAACAGGGCCACACCTTCTAATAGTGCTACTCCCTGGGCTGAGCATGTACAAACCATCACAAGAGGCTTACCTAGATTGCAAGAGATGTTTG
GTTTTGATCCCCAGGACCACAATACACTCACCACATGCCTATACACCATACATACATACACACACATACATGTACCATACATACACCATACGTA
CATACACACACACAAACTCCCCTCAGTTGGGGATATAACTAGATAGAACAGACTCATTAGAAATTCTCTTAGGCAGCTAGGTATGGTGGGAAGG
CTCCTGAGTATGAGGTCAGCCTAGCTTACAAGCTCAAAAGGAAAAAAAAAAGAGAGAGAATTAAGAAACTAAATATTTGTATAGATGTCTGAGG
AGAGAGGCCACTGCACAGGTTTGACTGGCTAAACTAACTGTGTACTGGGATACAACCAAAATTCTAGTGTGTGTGTGTGTGTGTGTGTGTGTGT
GTGTGTGTGTGTGTGTACACTTGATAAGTGCGGTCCGTGGATCCCATAGGCCTTTAGAAGCATTTCTCCTAAGAATGGGGACATTTTCCTGCCA
ACTGGTTTCATCATTGCAGATAAGGAAGCACAGTGATTGCCAGTATTCCCTCCGCCCCTCCCACCTCCCCAGTATCCTCCGCCCCTCCCGCCTC
CCCAGTATCCTCCGGCCCCTCCCACCTCCCCAGTATCCTCCGCCCCTCCCACCTCCCCAGTATCCTCCGCCCCTCCCGCCTCCCCAGTATCCTC
TGCCCCTCCCACCTCCCCAGTATCCTCCGCCCCTCCCTCTTCCCCATAGCTTAGAGCAGGGCACAGCCCCAGTGGCCTCCGGGCATCCCTGGGA
GGTGTGACGACAGATGGGTCTTATCAGGTGGGTCACACTGTCACCCCAGCTCACACTTCCTAACCTGACTGGTGGCCTTTATTGTTTTAACTGG
CCCGTTGGTCACTGCAGACACTGTGAGGTCCTGTGTGCTTGCTGGCCTCTGGCTTCCAGCCAGCAGTCTCTGGCACAGCTAGGAGCTCCTGACT
GTCGTGGGCAGCCTTGCTCCTGGCCTCTCCAGTAAGCAAAGTGTAAAGTGATGGCCTACTTGGATTGTCTCAGCAGTCCTGGAGTTCTGATAGG
CAGTCCTGTGGGTGGCTCCTGTCCCTTTAACGGGTTGCATCTGCTGATTTCTTCTGTGTGTGTTTAAACGCCTCTTTTAAATCCAGAAGTGAAA
ATTCTTCACCTTCGTTCTGTGGGCATTCCCCCCCCCCCCCACACACACACAGTTTGTCACTTGCCATTGAACTGTGTCTCATTTTTTCCCCCAC
ATGAGAACATTCTGATTCATTATGTGGTGATGTTTGGGTGGGTCTGATTCTCCTTGGATGGTGTTGGTTTCTTGTTTAGTTTTTTTTTTTTTTT
TTTTTGTCAGCTTGGCAGAAGCTGGACTTCTCTGAGAAGAGGGAACCACAGTTGAGAGAATAACACCTCATAAGATAAGATAGGTCTGTAGTCA
AGTGTCTAGGGGCATTTCACATTAGTGGTTGATGTGGGAAGAGCCAGCCCACGGAGGGGTGTATAAGAAAGCAGGCTGAGCAAGCCACGATGAG
CAAACCAGTGAGCAGCATTCCTCCATGGCCTCTGCCTCAGTTCCTGACTCTAGACTGCTGCTGCTTTGAGTTCCTGGCCTGCCTTCCCTCATTG
GTGGACTGTAAACTGTGAGCTGAAGTAAAGGCGGCCATTTATCACAACAATAAGAGAGCTAAGACACGTGAAATAGGTTTCCTTGTCAGGTGGA
AAGAGGTTATCCCAAGATTGTGTTACCTGCTGACCCTCATGAGCACACTGTGCCAGAAGCATGGACAAACACATTTGTTCTGTCCTCGCACAGT
GCCAGAATTTATTCAGCAACAATAAATTCCCAACGATTAGCAGTTTAGTAACATCAGGTTATCACTTAATCCTGCTTCCCTCAGTGATCAGGCC
GAGCAAGGCAAATGCAGATTCTTTATTCCCGTCTTTTTGTTTGTTTGATAACCATATTTCTCTGTGTAGCCCTGGCTGTCCTGGAACTTACTTT
GTAGACCAGACTGGCCTCAGACTCAGAGATCTGCCTCCCAAGTGCTGAGATTTAAAGCGTTCGACACCATGCCCTGCTCTTACCTGAATCTTAA
TTAGTAACGTTAACTGTCCAACCACACAGCACAGAGAAAATATGTGTATATGGCATGTATATGGTTATAGAGGGAATACAGGTTAAAGTCGCTT
GAGGTTTCAGACCTGGGCTCAGAGAATCTGCACTGATTTCAAACCAGACCAGTGCTGCGAGGGAGCCGCTGCAGTCAGGTGTTAGAGAGCTAGA
CACCTCCGAGAAGCTGGAAGCTAGAGTCTCCCAAGGCAGAAGCATGTCTCTGCTGAACAAACCAGGTAGCAACTGATAGTTTCGTGTGGGGCTG
TCCATCATAGTGTTGGGTGTCTATCACTTTAAGGAGACAAGGTGAGGATGTTACACCTAGGTCTATTAATTCGTAGCCCTGGCTTCCTAGGCCT
TTTTCGTACAACTGTGTGCCCTGACACACTCAGCTTGTATGAATTCATCCAATGGTGGCTCCCCTTTCTGTTCCCAGGAGTAAACCGTTTATCC
ATCTGTTTGGAGTGGATGGTGCATGGCAGACAGTGTAGCTTGCAGGTCCACCCAGGGCCTACTCATCCTTTTCCCCAAAGTGGAATCAAAAGCT
GCTTGTAAAACAGACTCTCCTAAGGCAAGGCACAGCCTGGAGACCGTTTTATACAATATGGGGCAGCTTAGAGCCAGGCACAGCCTGATCCCGG
CATGACCTGTGTTGTGGTGAGCTTGCTTTAGTAGAATGGCTTCTTCAGGAGCTGCTGGTGGTGGATGAGCGGGGGTGAGGGTTCTGTGGGTTGT
TTTCATTGGGTTTGTGTCGCCTGTTTTGTGAGGACTGTAGTTGAGCAAGCAACAACTGACCCTGCATATCTCAGGCTGTCACACACATGAGATG
TCCTTCAGCTGTCAACGGTCCCAGGGTGGGACCCACATGGATCAGGGCCCACTTACCCCACACTCTGGTCTTCTGAAGTGAAAGAATCTGTACC
TTTAAGTGATACAGGGACAGGTCCTCAGCACCGAGAGGTCAGATGTTTTCTTTTTATTCCATTGTAGAACTTGACATCCTTCAAGTTTCTGGGG
AAAAGTGAAGATGGCAAAGGAAGATGCCCCTTCGACCCCGCCCACAGCTACACATCAGTCATGGTTGGTGAGTCCTGCGTCTGAGGCTCTTTCA
CTCTCTGAGTTTATGGTGGCATGTGATGTTGCCACCTGGGACACTCAGGTCTTCCCTCTTCAGCCCTGCCTTCCTTGGCATCATCTTGGCACGA
GCTCCCTTCAGAGAAGGTGAGCGACTTGTGGCACAGACTCCTCAGTGAGAGCTTTCGCGTTTCCCATTCAGAGGGAGACTCAGGCCCCACCCTC
TGCAGCAGCATATGGCTTCTGGCTGTCGGTTTTTGTGCAGCACACAACCTGGATACTTGTGTGGCTCTGTGCCTCCCGCTGCGGTCCTGCATAT
CTGAGGCTAGGGGTCACACGCTGTCTTACAGATAGTTCATGTTGGAATCTCTCAGCCAGTGTGAAGGTTCAAGCTTCACTGTCAATGTATAACA
TTCAGTGAACTGTGAGATAGTTAATACTATATTACAAAACAGGCGTCATGTGTGACACTCCTGCGCAGTCATAGGCTAAAGTATGGGTTCTGAG
TATATTAAAGTAGACGAGCCTGGACCATGCTGCCTGGCAGTGTAGGTGCCTGCACATCTTTTTTTACTTACAGTGCTTTTTACTTAGGATGGCT
TCCCTGGGTGTCAAATACTGAGGAGTGTACTTGCCATTGCTATACTGACTGTGGCAAATTCTCATTTGTGATTAGCTATTACCCTTTAACCTGG
GAAAAAACAGTTAAATTTGGTGACTGATTGTTCTGATGAAAGATGCTGATGTAGGAAGCAGGAATGCATGTCAGAGAAGACGAGGACCCCCCCC
CCCCCGCCATCCTATAAGGAATTAATGAGGGTATAAATGAATTATGGTAGCCTGGTATGGGTGTGATAGCCTAGCAGAGGTGGAACTATGAGGC
TTGCTTACAGTCCAGCCTGGGCTAATGTCAGAACTTGTGTTAAACAGTTGTCCCTAGTCTCTGACAGAGGCTGAAGCTCTGATTGGATCTTCTC
TACTCCCTCTCCTCCACCACACGGGGCGAGCTCTACTCTGGGACGTCCTATAATTTCTTGGGCAGTGAACCCATCATCTCTCGAAACTCTTCCC
ACAGTCCCTTGAGGACGGAGTATGCCATCCCGTGGCTGAACGGTAAGTGTGTGCTGCCAGCCTTGTGGGTCTACCATTTTATTCTTCCTCTAGC
TGTCCCCAGGTCACAGGAGCCGGGGGAGGCGGGGGTGGGGTGGGGGTGGGGTGGCGGGCTGCTCATGTCAGATCCTAGGCAGTGCTCTTTGGAA
CTGCTCAAGGCCCGCACCAGAGTAGCTGTCTTGCTCACTGCTAATAGCATTGTTTAAATCTGATTCTCTGACATACCCCACCCAGCCCCACTTC
CACCCCGTCCCCCAAATGCAGTTTGGGAGACTTTAATAAAATGAAGCTCTAATGGGAAGTTGAGAATGTAGACACTGAGTTTGGGTGTCATCTT
AGATGTGGAGACTCTTGGGTCTGGCCGGGTGGTGTTGAAATGTGTCTTTACTCCCAGACAAGCATATCAGGGTGTGGATGATGGGGGCCCTTTG
TGGGTGTCTTTTTTGTGGGGATGCTGACCAGTGATGCAGGGCTTTTTCGAAGCCCCTTGATCTGGAGTCAGACCCTGGAGTCTGAGATCTGAGA
CAGCCAATTTCTCCTTGGGCCTCCCCAGGAGTCTGGAAATCCTTCGGGTGGCTGATTTGGCCCTTGTTTAAATTGGCAGATACTCTAGCATGCC
AGACCAACCACCAGCAGAGAGGCCTCACCCGGCTTCCTCTGTGGCTATCTTGGTGAAGCTGAGGAGGGTGGCGGGCGTCCTCTGGGCCTGGAGT
TCACAGTGTGGCATACATACTGTAGACGTCCTAGAAGGGAAGCTTCCAGGCTCTAAGGCTGCCCTTGCCAGCCTGCAGCACCCCAGGGAGCAGG
TAGAGAGGTGGCTTGGTAGCTCCGTGTACAGAAACCATCCTGTGATGCCACCCCACAGAGCCTAGCTTCGTCTTTGCTGACGTGATCCAGAAAA
GCCCAGATGGTCCGGAGGGTGAAGATGACAAGGTCTACTTCTTTTTTACGGAGGTATCCGTGGACTACGAATTCGTCTTCAAGTTGATGATCCC
GCGAGTTGCCAGGGTGTGCAAGGTGGGTGCTGTCTCCCTGTTCCCACCCACAGTCCTGCATGATGCCACTCCAAGATTCCTCTCTGGAAGTGTG
GCCATCTCCTATGGGTAGAGACCCCTGCGGCAAAGTTTAATGTACAGTTGAGGCACAGTAAGAGACCAGCAATAAGAGTTTTAGAGGTATGTGC
ATGGTTTCTCTTGAGACCCAAAGTAGGATTGGGAAGATGGCTTAGTGGTTAAAATGCTTACCACGGAAACAAAAGGACCAGCGTTCAGATCTCC
AGAACCTATAGATGCTGAGCGGATCCTCTGTAGAAAAGTGGAGATAGGGCCTGCTAGTGGGGCTGTCCTCATCGGCAAGCTCTGAGTTTGATTA
TGAGACCTTGCCTCAAAGAATAAGGTGGATGGTTCCTGACATCAACTTCTGGCCTCCAGATGCACCCACACACGAACACTCATATAGACACACA
CCGCACATACACACATACATGAAAGGAAAGAAACTCAAAGAGGAAAAAATTAGTCAGAGTGAGCACTTGTGATATTTTCAGTGTGTCACTAATC
ACAGGTAACTGAAATTGCAGAAAATGCAATCACGCACACGGGACCCGCCTTATTTCTTTCCAGCCCAGAGTGGAGTATAGTTTGTAACTGTCTG
TGTTGTCTCAGTCGTCAGCTCCCTGAAAATGACTAGGATTGAGCAGGCAAAGAGGGCACAAAAGGAGCTCTTAGTGGATGATCATGTGTTTGCC
CTAGGATGGTCTGCGGGGCCAGGGTCTGTGGTGGGAAGGAGGCCTCCCATCACTTGTTTAACTCCTGGGTCACAGCTGGGAATGCTGCAGGAGC
CAAAGCCAGTGGCACGCTGATGTGACACAGATGGGGTGGTTTTGCTTGTCATTGAATGTGTTTGGCTCTTTTCGTATTCTGTGATCCTGTCCCA
CATGGAAGTATTGCTTTGGTTACATAACTTGTCTTCAGTATTCTTGGGCTAGGCCGTGGTTGGGGACATATGGACGCCTGCTTCTGTGGCTAGT
CATCTGTCCTGCCTCTACCATGTGCTGTCTCTGGAATTCCAGGGCGACCAGGGCGGCCTGCGGACTTTGCAAAAAAAGTGGACCTCCTTCCTAA
AGGCCAGGCTGATCTGCTCCAAGCCAGACAGTGGCCTGGTCTTCAACATACTTCAGGATGTGTTTGTGCTGAGGGCCCCGGGCCTCAAGGAGCC
TGTGTTCTATGCGGTCTTCACCCCACAGCTGTGAGTTCACTTGTGAGTCCCTTGGCAGTTGGGGCCTGGTCACCCTGACGACCTCCTGCCCTCC
TGACACTCTCACGGATTCCCCTCCACAGGAACAATGTGGGTCTGTCAGCGGTGTGCGCCTACACACTGGCCACCGTGGAGGCAGTCTTCTCCCG
TGGAAGTACATGCAGAGTGCCACAGTGGAGCAGTCTCACACCAAGTGGGTGCGCTACAATGGCCCCAGTGCCCACTCCCCGACCTGGAGCGGTA
AGCAGGGTCTCGAGCTCTGGGGAGGGCTCATCTTCTCTGGATCTCAAGGCCCCCCAAGCATGAGACCACACAGATCCTAGTGCATCCCGGCAGA
GGGTTGCATGGCAACAGGAATCTGGGTGCTGGGCTGAGTAGCTGCCGGGCCTGGGGTGAACAGTGAAGCTGTTCCTTTCTGATCTCCTCTGTGT
TGGGTAGCAGGCCTCACTGCATGCTGCCCGGACGAGAGAGTCTACCGCCGGGCTCCTTTGAGAAGTGGACATTGCACAATGGCAGGCTTGCTTC
ATTTCCCATTTCCTGCAGACAAAAAAAACCTGTTTGTCAGCCTGGGCTGTCTCCTGAGGCAGCCTGTCCACTAGATAACAGGAATCATATTCTT
TCCTCCTAGATACAGTAAATTAGTAAAGAAAATTCTAGGGTTGCCTGCATATTGTTGCCACGTGGTGGATGGGTGTGGGTATGGAGGGGCTTGG
CTCTTGAAGCTCTCCCAGGATGGACTGTTCTGTGAGTGTCTGGCCTCGACACCTGGTTATTCTCATAACTCATAAATTCAGGTGTTCAGTAAAC
ATGGCTAGGGAGCTGGGACCAAAAAAAAAAAACTCTTTACAACTTTTTTTTCTTTTGTGCTACCCAGAATCTGGCTTTCTCTCCTCTCTCTCCT
CTCTCTCTCTCTCTCTCTCTCTCTCTTTCTCTCTCTCTCTCTCTCTCTCTCTCTCTCTCTCTCTCTCTCTCTCTCTCTCTCTCTCTCTCTTTCT
CTGTGTGTGTGTGTCTGTATAACAATATTTAACAAGCTTCCTTTGGCATACTTTCTTCTCATAGAAAAGCTGTGAAATGAGTGCTTTTTAATCT
GGAAGTTTGCTAAGTTTTTTCATTGGCTACTAATTCCTCTACTGATCCTGAGACAATTATGTCCCGTGGCTTTAAAAGCACAGCTCTTTGTCTC
TGGGGAGGTCACCTTAGCCCCTGTTACTCTTTGGAGAAGTGTGACCATGGCCCTCCAGGGCTTCTCTTCTGTGTGCTCAGTTGAAGCTAAGTGG
GCATGTGACCGCGGTGCTTTTACCCCACAGTGTATCGACAGTGAGGCCCGGGCAGCCAACTACACCAGCTCCTTGAATCTCCCAGACAAAACAC
TGCAGTTTGTAAAAGACCACCCTTTGATGGATGACTCAGTGACCCCGATAGACAACAGACCCAAGCTGATCAAAAAAGATGTAAACTACACCCA
GATAGTGGTAGACAGGACCCAGGCCCTGGATGGGACTTTCTACGACGTCATGTTCATCAGCACAGGTGGGTTCCTTGGTAAGATTGGGAAATTC
ACTTTTAGCCATCTATAGGTAGGTTTGGTCTCTCCAGGTACCAGGAAGGTTTGTGACAGCAGAAAAGGATTTCTGAGGTATCACAGCACCTGGG
ATTATGAGTCTCTGTGGCCATCAGGCACCTGGGGCACTGGTTACTAATGCAAGTCCCAACCTGTCGTACCTGGGCTGGAGTGGAGTTAAATAGT
CCCTGTTGTCATGGGACTAGTGGGACTGGCCTGGTATTTCCTAATTGGTAGTGGGAGACTCTGCCCCCTCCTAGGTAGGAGCCAGGTTCTGTCC
TGCCCCGAGCTGCCTTTGGAGATGCAACTCTCTCTCAGATGTCTTCTGGGATGGGGATGGAAAGTTGGAGCATGGCAGCTGCCAGAGCCCCAGG
GGCTACCCTTAGAGCTTCCCAGATGACTTGAACATTTTGCCCTCGTCTGCCTGCCTGGGCCCCTAACCTCCCCTGGAAGACCAGTAAAGTGCAA
CGGCCACCTTATACTGAATGTGGCTCAGGTGTCAGGGTGTAACCTTGTTGGTGAAGGTCATTGGAATCCACCATAGGACAGGGTTGTCCTCTTC
TGAGTGCCCCATTGAGGGGATGGCTTGAGTTATCCACGAGTGTGAGTGGTTGCCGCCAAGGCTGGAGTCTCTCTCCTAGTTTCATTTCTGTTGC
CGGGCGTGGTGGCGCACACCTTTAATCCCAGCACTCGGGAGGCAGAGGCAGGTGGATTTCTGAGTTCGAGGCCAGCCTGGTCTACAAAGTAAGT
TCCAGGACAGCCAGGGCTACACAGAGAAACCCTGTCTTGAAAAAACAAAAACAAAAACAAAAACAAAAAAATTAACCAGGGTGCCACCCTGGGC
CTGTCCCTCTAGGGCCTCCTTGCTCACCTGTGTTCTGACCTTGCAGACCGGGGAGCTCTGCATAAAGCAGTCATCCTCACAAAAGAGGTGCATG
TCATCGAGGAGACCCAACTCTTCCGGGACTCTGAACCGGTCCTAACTCTGCTGCTATCGTCAAAGAAGGTAATGTGGGTTTGGTTGGTTGGTTG
GTTGGTTTTAGGGGTGGGGGTCAGGTCCAGGCGCTTGTCTCCCTTGTAGCTTCTCTGTGGCTAGGACTGGCTAGCATGGGTCTCTCAAGGCTTG
GCAGCCTCTGGCCCAGGTGAACTTGGCGTACACAGCATGGGATCTGAGGCTTGGGAGGAGGCATGGGAGAGCCAGGGGCCTTGATTGTACTAAG
CCACTGGAGCTTTTGGGAGGTCCCTAACCTGGGGAAATGTCTGTGCCTTGAACCTGAAAAAAAAATGCAGATTGTGCTGGTGGGACATGACATT
TTGCTAATTTGTAAGGAGAAATAACAGGGGCTGGTGAGATGGCTCAATGGTTAAGAGCATTGACTGCTCCTCCAGAGGTCCTGAGTTCAAATCC
CAGCAACCACATGGTGGCTCATAACCATCTGTAATGAGATCTGATGCCCTCTTCTGGTGTGTCTGAAAACAACTACAGTGTACTTACATGTAAT
AAATAAATAAATCTTTTTTAAAAAAAAAAAAAGAGAACTAACAGGTCACCCTCTAGAGCTCCTGCCATACCGCCCTGTGCATCGGGCAGCCTTT
GCAGAGGCCTCAGTCACGGGGGCTCAGCTCTGCACTGTGGCTCCCACGAGCCACGGAAACACATAGATGACCAAACCTGCCCTCGAAGCCATGC
TATGTATTGCCCGAAGTTTTCATAAAAAGCTCCAGCCAGATGGTAGTAGTGCAAACCTTTGATCCTGGCACTCGGGAGGTGGAGGCAGGTAGAT
CTTTGTGAGTTCAAGGCCAGCCTGGTATACATAGTGTGTTTCAGGGCAGCCAGAACTATGTAGGGAGACCCTGTCTCAAAAATGAATGAATGAA
TGAATGAATGAATGAATGAATGAATAGGTATTTTTTAAGCCCCTAAGTGGGGTGCAGTATGGTTCATACCTCTAGTTCCAGACCTTAGAACATT
GGAAGCAGGAAGATCAGGAGTTCAAGGCTAGTCTTAGCCATATAGCAAAGTCAAGGCCAGCCTGAGCTAAGTGAGACCCCATTGCAAAAAATAA
AACAAGGTATATAGGAGTGAATAGAAATGGCCTGGGTGGGGCTGGAGAGATGGCTCAGTGGTTAAGAGCAGTGACTGCTCTTCCAAAGGTCCTG
AGTTCAATTCCCAGCAACCATTGTGGCTCACAACCATCTGGAATGGCATCTCTGATGGATGCCCTGTTCAGGTGTATCTGAAGACAGCGACAGT
GTACTCTATAAATAAAATAAGTCTTAAAAAAAAAAAAAAGCCTGGCAGTGATGGTGGCACATGCCTTTAATCCCAGCACTTGGGAGGCAGAGGC
AGACAGGTTTCTGAGTTCAAGGCCAACCTGGTCTACAGAGTGAGTTCCAGGGCAGCCAGGGCTACACAGAGAAACCCTGTCTCAAAAAACCAAA
ATGAAAAAAAGAAAAGAAATGGCCTGGGAGCTGGGCTTGGTCCACTGGCTGGAACTTGTTCCTTGTCCTCTGGCCTTGGGAAGATGTGGACACC
ACATTTTACATCTAGGATAAAACAAGGAAGTTCAGAACATGGGTTGTCTCCAAATCCTGACTACACTGCCTGCTGTCATGTTACCCTGGGCATC
ACTATATGGTCCTCTTAAGGTGGTCTCTGTGTGTGTATGCTTTTCATTAATGTGACTGTTAAGAGGACATGTGAGCCTCCTAAGTGCTGAGGGC
TGGCTTTGAGCAGCACAGAACCCAGGGAAAGCTAGGAGCAGGATCAGCTGCCACCTGCCCTGCTTCATGCCTCCTGGCCAAAGGTGCTCCCCTC
CCCTTGCTAGGGCTCTGCTGTACCCCCCTGAGAAGATGGGTCCAGGGCCACACTTTTGGCTTCAAGTGCCAGTGCTAGCCTCTTGCCTCTACCC
TTGTCTCTGATGCCTGGCTCTGGGACACTGCTGAACTGTGGCCTGGTGTGACTGTCCCCGTGGACTGCACATCTGTGGGCCCTGTGTTTCCAAT
TCAGTTCTCTTTAAGTAGCTGGCCACAGCAGTTACCAAGGGTCCATCAGTCTCACTTACCCGTGTGCTGGGCTTCTGCTAGCACCAGGTACAAT
GCTGGTCCTGGCTTGCACCCAGGAACATGGCACGCTCCTGTCCTGGAAGTCAGAGCGATGTTGATAGGCGTGGTCACATTGGCAGGCTGGATTC
AGAGATGACTGATAAGGATGTGTCCTTATCACAGGTACCTACTGTCTGGCTCAAGACAAGACTGGCATTTAAAGGTTGTGCCCTGTGGATTCCA
GCTGTAGTGCTGTGTCACTTACAGAGTCCAGGAGAGCAGTGGGGGGGGGGGGGGTCACATTAGGAGTGGGAAGCAACTGGAGAGGGCTTTTATG
TGGTTTTCTCAAGACACAAATGAAAGTCTCTTCACCCAAGATAGAGCACCAAGGATAGACCAGTCGAGCTCAGTGAGCCAGTGAGTTTTGGGGA
GGTAGCTACAGGGAAGAAGGAACAGGGGATTATTTACAAGCCCGTGGGTGGTTCGGAGGCAGCTGCATCCATATATCTCCCTTCACAGCTGGTA
GGCAGCTCGGCAGGGCAGAGTCTCCTCTCTACCGCAATTGTGTGCTGCTTATATAACCTTGGGGCAGCGGGCACTTGTGAATCTTAAACCTCGT
CAGGCTTCCTAAATTTCCTTCACTTTGAGTCTTGTGAGGCTCCCTCGGCGCTCCGGGAGGACGTGTTTCGATTGGGCAGACATAGAAACAGAGC
TGTCTCTTCTGGGAAGAGGAGCTGCTTTGGGCGTGCAGTGTGCTTAGCCCAGCTACCGAACGGTGGCGTCAGATCCCAGGCCCTGCAGTTCCTC
CACTGGCGTTTGAATCCTCCTGGGACAGGGGACACCCTGCTTTTCCAAACAAGATTTCTGTCTTAATGTGCTTTTCTGTCACTGCGATAAACCA
CTCTGACCAAAACCAGCTTGCAGGAGGAGAAAATTTCCTTCGGTTCACACTTCCAGGTTGTAGTCGCTGAGGGAANNNNNNNNNNNNNNNNNNN
NCCCTGCCCTGCCCTGCATGGCATCACTCACCCCGTGCTCAGCAGACCAACCTTTCCTTGAACAGGCTAGCTACCTTTCCTTGAACACCTAGCC
TCGTGGCCATCAGGTATACAATATAAGGCTTGGGAAGAGCCAAGGTTTTGGCATTTGGTGCAAAATTTGGATCTATGCCCAGCAGAGAAGAGAG
GAAACCCATCTATTACGTTTTCATATTTGTTATCGTTTTATTTGTAAAATGTCACCCATGGGCTCATGCGGGAATGGATTATTAAGCTTGCCCG
CTGGCTTCTGCTTAGCTGCTATCTTATAAAGTCAGGGGTGGCATCACTCACACTGGGTTGCACCCACCCTAATTACTTAGCAAATAAGAAAATG
GCCCACAGGCTGGGCGGTGGTGGCGCACGCTTTTAATCCCAGCACTTGGGAGGGAGAGGCAGGAGGCCAGCCTGGTCTACAGAGTAAGTTCCAG
GACAACCAGGGCTACACAGAAACCTTGTCTCAGAAAAGAAAAAAGAAAAAAAAAGAAGAAGAAAATGTCCCACGGACATGTCCACAGGCTAATC
TGATATAGGCAGTTCCTCAGCTGCAGTTGTCTGCCCCGCAAGTGTGGCAAGGTGACAATGAAAACCCATCAGGACACTGTCATTTAGGTCTTAG
CGGTCGGGCCTTGTCGCCTATTCTGAGTCAGTGAGTCCTTCCCTCCATGCAGTGTTTGACTGTCCTCTCTTCCCAGGAGCTGGTGGAGAAGCTA
GCAGCCCCTCCCAGTGCCTGAGTCCTCCTTACACTTGCCATCCGTCTCTCATACACAGGGGAGGAAGTTTGTCTATGCAGCCTCCAACTCTGCA
GTGCTCCAAGCGCCCCTGGCATTCTGCGAAAAGCACGGTAGCTGTGAAGACTGTGTGTTAGCACGGGACCCCTACTGTGCCTGGAGCCCAGCCA
TCAAGGCCTGTGTTACCCTGCACCAGGAAGAGGCCTCCAGCAGGTATGCCTAAGGATGGGGCTGTGTGCCTATGCCCCATGCACAAGCCATAGA
GTAATGTGCGCCTGCTGTCTTCTAGGGGCTGGATTCAGGACATGAGCGGTGACACATCCTCATGCCTGGGTGAGTGGATCTCCTTTGCTTCCCA
CCCCTGGGCTGGCAAAGCTCCCGGGGCGTCAGTTCCATGGTTTTCCTGTGCGGTCTGTCTTCCTTTCTCCTCTCTGAACAGAAGTCAAGTGTCA
CGCAGCAGACCTGTGACCTCTCCTTGTGGTGAACCTGTGATCTTTCTCTGTCCCTTTGGTTACAGAAAGAGGGCTCAAAGGCACACAGGGTGTA
TCGGTTCACTGGAAAGCACACCCTGCCCCACACCATGCTCCTCTGTTCTGCTTAGCTGGGCCCCGTAAACAAAACTGGAGCCTGCTAGGAATGA
GAGCGAAGGAGCCCTTGGGTATGACCAGATAGCCCTAGCCTAAAAACAATACTCAGGAACAGTGTGCGACAGTGTCATGCTTCAAGATGAAAAT
CCTACCCTAGAAAGAAGTGCCTTAAACGCTAAGGAGATGGCTCAGTTGGTAGGATCTGAGTTCGATCCCCAGAACCCATGTAACAGCTGGGCAT
TGGTGGTTCATGCTTGTGATCCTAGCCCCAAGGAGGGTGAGGCAGGTGGATCCCTGGAGCTTGCTAGCCTCACACAGCCTAGCCGACTTGGCAA
GTACCAGACAAATGACAGACTCCATCACAGAAAAAGACAAAGAGGGTAACTGCTATCTGAGGATGATACTCAGATTTCTGGCCTCTTTCTATAT
AAACACACATGTACGTACACACACACACACACACACACACACACACACACACAGAGCAAATGGCATCAGATTGCCTTCAGGCATGTGTAAATGT
CACACAAATTTCATGTGTAGCTTTCAGTCCCCTTCCAAAGATAGTTGTCTCGTTTTATATATGCAAATATTTCCAAGCCCAAAGCATTTTTCAT
CTTCCGTATTTCAGACAAAAGACACAGTTGTGGTTAGTTCTGGTGTCCTCTTGGGGCACCCCCTCCTGATGGCTGGCCATCAGGAAACGGCTGT
GACCAGAGGTAGGCATAGAATCTTGCCAGAATTCTAGTCTGCCTTCCCAGCCCCGTACCCCCTTCTCTGACTAGCCACATCAGAGTAAGAAGAG
GGTCCCTGGTTGCTAGCTCGCCTGCTTTAGCCATTAGCAGAAGTATTTGTCAGCTGACTGGGTGCCACTGTGAGGGAGCTGAGCCGTGCCCAGC
CCCTTCCTCAGCCTGCTGACCTTTCTCCAGGGCCAGGGTGAGAACAGAAATTCTCAGAGCTGTCGCCAGTTCCTTCAGTGTATGGAAGGGGGAC
GGGGTGGGGGCTGCTCTAGGGTCATGGATGTCTTTACCTTGGTCCTGAAGATTGCACCTGGCTCCAGTGGGTGGAAGTCTCATGCCGTCCTGTG
GGTATTAACCTGTTTTTTGAAAGCAGTCGAGCGGTCAGACGGAGTTGTTGACGGTGAGCACGTTTATTTTGATGTTCCAGATAAGAGTAAAGAA
AGTTTCAACCAGCATTTTTTCAAGCACGGCGGCACAGCGGAACTCAAATGTTTCCAAAAGTCCAACCTAGCCCGGGTGGTATGGAAGTTCCAGA
ATGGCGAGTTGAAGGCCGCAAGTCCCAAGTACGGCTTTGTGGGCAGGAAGCACCTGCTCATCTTCAACCTGTCGGACGGAGACAGCGGCGTGTA
CCAGTGCCTGTCAGAGGAAAGGGTGAGGAATAAAACGGTCTCCCAGCTGCTGGCCAAGCACGTTCTGGAAGTGAAGATGGTACCTCGGACCCCC
CCCTCACCTACCTCAGAGGATGCTCAGACAGAAGGTAGTAAGATCACATCCAAAATGCCGGTTGCATCTACCCAGGGGTCCTCTCCCCCTACCC
CGGCTCTGTGGGCAACCTCCCCCAGAGCCGCCACCCTACCTCCCAAGTCCTCCTCCGGCACATCCTGTGAACCAAAGATGGTCATCAACACGGT
CCCCCAGCTCCACTCAGAGAAGACGGTGTATCTCAAGTCCAGTGACAACCGCCTGCTCATGTCTCTCCTCCTCTTCATCTTTGTCCTCTTCCTC
TGCCTCTTTTCCTACAACTGCTACAAGGGCTACCTGCCCGGACAGTGCTTAAAATTCCGCTCAGCCCTGCTGCTTGGAAAGAAAACACCCAAGT
CAGACTTCTCTGACCTGGAGCAGAGTGTGAAGGAGACACTGGTCGAGCCTGGGAGCTTCTCCCAGCAGAACGGCGACCACCCCAAGCCAGCCCT
GGATACGGGCTATGAAACGGAGCAGGACACCATCACCAGCAAAGTCCCCACGGATCGTGAGGACTCGCAACGGATCGATGAACTCTCTGCCCGG
GACAAACCGTTTGATGTCAAGTGTGAACTGAAGTTTGCAGATTCGGATGCTGACGGGGACTGAGGCCAGCGTGTCCCAGCCCATGCCCCTCTGT
CTTCGTGGAGAGTGTTGTGTTGAGCCCATTCAGTAGCCGAGTCTTGTCACTCTGTGCCAGCCTCAGTCCTGTGTCCCTTTTTCTCTTGGGTTGA
GCCTGTGGCTCATCCCCTTTGTCCTTTTGGGAAGCAAGTATCTATTCCAGTCTCAAGTCCTGCAGTTGCTGGAGCGCTTACGCACCTGAGCCCT
TTGTGTCCTGGGGGAGAGATGGCCACCTCCGTGGGCTGCGAAGAGCCACCCCTTCCTCTTCCGATTCTCCTAGCAGCCACTCAGAGATAATTTA
ATTCCAGATTGGAAACGCCCTTTTAGTTTATCAGATTGGTAACTTACATCCTGCTCCCCAGATGGCACGGACAGTTTTCTTTCACTTAATTATT
ATTTTTTTTTTAAGGATTTTCGCTCCTATTGTGTTGATGTCTTAGGTCATTTTCTTTTTTTCTTTCTCTTTTTTTATTACCAGAGGAGATGTTT
TAATATTCATGAGAAGAGGAACATTTTCTAGATTTTTTTGTTGTTATATATTGAGATATAAAATATGGCTATGTTGCTTAAGATTCTCAGGGAT
AGACTTATTTTTGTTAACTTCATTCTTTCCTGCTCTTAGGAACATAGGCCTAAAATTGTCTCTTGAGTTTGCTCACCCTTTTGTTTTGGTAGCG
TTTTTTTGTTGTTGCTGTTATTGTTTCTAGTTTTTAATCTTATTCATTTTGAAGGATTTTTCTTTCTGAACTTTTTAAATTTTTATATTTTCCT
GCCATACATCTACAAAGTGGGTTTTGAGTGAGGGCAGGTGGCCCAGTGCCTTTGGGTGGCGACTGAGCTGGTCCCACGAGGGGAGGAGGGTTTA
TATACCCCATGACCCTGCGGCTTCTTGGCGCCTCCTGCCCATGAGGATCACATCCTGTCTCTCCTTGCTTCCATCTCTCATCACTGCCCTTGGA
CTTCCGCCTTGACTGTCCATGAAAGACAGAAATGGGTTGGGTAGTTGGCCTCCCAACCTCGGATGGTGACCGCAACATCCCGTGTGGGCGGCCG
GCCGCTCCTGCAGCCCGACTCTCCTGCCAGTGTCTTTCAGGATGTCAACGGGTGGTACGATTCTGGCATTTGTTTCTCCCTCACCGTGTGTGGA
ACACTCATTCCATGTAGAGGGTGACGAACTCTGGATTCCCCCCCACCCCCACCCCGTGCCGTGTAGACACTCATCTTCTGCATGACATGATCTA
CCATTCGGTGTAAACATTTGTGTTTATAAGATTTACTTTGTTTTTATTTTTCTACTTGGAACTGTACACATTTGAAAAGTACCCAAATAAACCA
GAAGCTTTATCGTTGACTGGGCCAGCCTTTGCCTTAGTGTGGAGGCTGGGGGCGGTGGGGTGTCCTCCTGTCCCTCTGTGACGGCCCTGGGTGG
GGGTTGGTCCTGGGATCTTGTCTGTTCTCATCCCATGTGTCCGGTTTCTTGCTTTACCTTCTCCGTCATGGGAATGGTTCTGACGACTTCTTCC
TCAGTGCCTTCTTTTGTGTCCTCTGTCTCACAGCCTCATCTCCTAAGTCCTTCCCTCCTCCTGGCTCCTCTCTATCCTGTCTGGGCCCTGACTC
ATCTTCTCCCAGAAGCCCTCGGCCGGCCTTGGCCTCGGGGCCCAACAGCCGTAAGCAGGTCACCTTACTGCCACCCTTCTTGAGTGACCAGGCG
CAGAAGGTGCAGGCGCTGGGGACCTTCTACCTCTTCTGCCAGGCCACAGGTGAGTTGAGGGGCCTGAGTGTCCATGGCTGTCCCCTCGCTCTGC
CCTTGCTCCTAGCTGTGCCTGAGGTTCAGCTGTCTGTGCTGATCCCGGGATCAACTGTCAACCCTGCAACGCTGTTGCTAGGGCTGCCGAAGGG
GTCATCCAGCCTCTCGCCGTAAAGGTACAGGTGTTGGGTCTTCTGAGCATGTAGGCTGCTGTGCCCAATGTTGTATGTCAGAAAACTGGACCTA
CAGGGCAGCAGAGAACCAGATGAGGCCACGATTTGCTAACCTGGGTTAGGTAGGGAATTCAAGGCCAACCCCAGGCAACTTATGAGAACCCTGT
CTTGGAGTTTTAAAATAGGAATTAGGATACATCCAGTGGTAGAATGTGTGACTAATGTGTACAAGGTCCCAGCCACAGCATAAATAAATACATA
AATGCGTGAAGTCCATAAGGTGGAGGTTTTCTCCCTCTGCTGGAGACTATCACATAAGGACATGGACTCTTGAAAGATGGTAGTTTACCCAAAC
CATTCTTTAGCTCAGAGATACCCATAGCCCCATCTTTCCCTGTGGGCTTTGGCCTCTGCACAGAAGTGAGGAATAGAGTTGATTTCAAGACAAA
GCAAATCCGGTTTATCTTAAAATTGCTTTTTAATTGTCGAAGCTATGGAGATCTTGAAAGTGCTATGTGTTTTGTTAAATTAGCTCTAGGATCC
CAGGGGCCTGTGTTGTCTGCTGTGAATGCAACATGCCGTTGGCAGCTGGAGGCCCCGTGGAGGCTGTGGGAGGTGGGCAGAGGTTGCTGCAGAC
CTACGTACCCTGACATGTCATCAGTGAATACATGGGCCTTGATGGCCCTTTTGAGTTGCTGTCTTAACTCTCGCTGCCCAGGCTGCTGACCCTG
GACACCTGAGCTTCAGCCCCCCTCTCCAGGTCCCTGGGTCTCCTGGTGTGTGTCACGGTGCCCAGTGATTCCCATTTTGTGCTTGCTGCTGTGG
ACCAAGCCTTCATGTCGACACCTGTTCTTGTTCCTCAGTAAGATATTTTAAGATAGATCATTTATTTATGCATGTCTACAAATCTGTGAGCGTA
TAAAGGGGCAGAAGAGACGCCTTGAAGCCAGAGTCACACATGATTGTACATCACCTGGTGTAGATGCTGAGGACTGAACTTGGGTTCAACTTGG
AACCTGCGAGAGAAGCAGGTGCTCTCAACTGCTGAGCCATCTCAGCACGTCTCTAAGGGGCAAGCCATTACATTTAGGGGAGTGTTTTCCAGTC
CCCGGTATTATTAGCAAGGCTTTCACAGGTATGGGTCCTCCGGTGTGGGAGAGGGTGAATGACTGGGGCCTTCCTTAACGAAGATAAGACTTCA
TTGTTAGATGCATAGAAACTACAACATCGGGCATTTCAACAAGGGGGAATTCGAAGCCGGGAGTTGTTTGCTAAGGTGATGGGGAACCTAGGAG
TGAGACAGGAAGGTGGGCTTCTCCAAAGACTGCTACAGGAAGCCACCAGCATGGGCAGAAGCAAAATAAAACCTCACTCACACTTGCCAGGCCT
CTGAGGTTGGGCATGACCTGCAAGGGTAGCCTTGCCGGCAATGTTCCTACCGCCGCACTCCTCAGGTACCACGAGAGACAGGAGATGCCCTTTT
TGAACTTTTTAGTGACCTAAGGGAACACTGGAAGCCACCGACTGAGGAGCCATGGCTGCGGACTGTGGGGGCCGGAAGTGTGCAGGGTGTGAGT
GGAAACTGAAGTGGAAGAGTGAGTTCCTCTCATGCCCCTCAGACCGGACTGAGCCCAGGCTGGGGAGACAGCTCACTGATTAAAGTCCTTACTG
TGGGAGTGTAAGGACTGGGGTTCTCATCCCCAGACCCCCACCTAAGTGCTGGGTAGGCTTGGTGACCCACCTGTAATTCCATCTTTAGAAAGTG
GTGATGGAATCCCCAGAGCATGCTAGCTGGCAGGATGAGCCATTTAGCAGTGCTCTGGGTTTGATTGACAGAACCTGTCACCTCAAGCCTTTGC
ATGCATCTACACACACACACACACACACACGCACGCACGCACGCACGCACGCACGCACGCACGCACGCACGCACCAAGTCTCTGTCTTGAGCAG
ACATGGGACCAAACCCTTACTCTCCCTTACCTACTTTCTCCTGTCCTTTTTTCCACAAACATTTGACCACACCCGGGGCCTGTCTTTTCAGTCC
TGTCCTCCACCAAGACAAGGCACTTTAGGGAAAGGCACCGCCAGCCTGGTGATGGCTGGGTGGGCTTTGGTGGTGTGAACATGCCAGGGCCTCT
GCTATCTTTGAGCAGGTGGCAGGTTGGGTCGGAGTTGGCAGACAATCTTATCTGAAGCAGAGGCCATGTGGTTTTGAACTCAACTCCCTGTAAC
CTTTCTACAAAAAGCAGTGTATGTCTTTGAGGTCTGCTGAGGAGGCCAAAGCAGCTGTCCCAGCTGACACCCCAGTTGGCTCTCCTAGCCCAAT
GACATGGGCAGGGCAGGGCTAGGTGGGCAGAGCAAGTTGGGACCCTTGACCTGCGTTTTTCTCTCTCCTAAAACAGCTGTTTGTATGATTTATG
TGAGTGGCTTGGGGCTAGCTGATTAAAGGCCTAGTGCGCTGTTTTTAGAACACAAGACTTAAAGAGAGGGTGAACAGGGACTCTTCACTCCCTT
GGCATGTGTCCTGGTGTCCGCTCTGGCTCTGCTGGTGACTCTGAGCTATCTTGGGGGAGCCTCACCCCTCTAGAGGGTTGATTAGAGGTCAGAT
AGACAGCAAGGCTTGTACTCAGGACTATTCTGCCCACTCCTGCTTCCACCCAGGACCTCCATGTCCTCTGGATTGGATGAGTGTTTTCCCAGTG
TATGAGGCAATTCAGACGCCCTGTTCTGCATACCCAAGTGAGATGACCAGGGAAGACATGAAAGCTGCCCCAAGCATGGCTGTGGGGGAAGGTC
AGGCTGCCTACAGCCTAAAGCTTGCAGGAACTCCCGTTTAGTTCCAGNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNTTTAGTTCCAGGAAAGGCAGCA
GGATCCGGCTCTGACTTTTCCTCCAGTCTTTGTGCCCGGGTCTGCAGCCCAGCACTGAGGTCCGGCCATACCCAGCCAGGTGGGTGGAGGGCTG
GAGCCGCTGCACTGTCTGACCTTACACTGCAGTTGCTTCCCCGTGTGCTGATATCACTGGGAGGTGCATGTTTATTCTGTCCTGCTGCCAGCTG
CCAAAGGTTGGCATATGTACAGCACATGCCATGCCAACACACACCACACTGTGACATACACTACACCACACCACACATACCACATATACCAGGC
ACCACACATAACTCCCACCACATACTCTCCACACATAGCCCCCAACACCACACATATAATTACCCACTGCACACACAACACATTTATCCCACAT
TTCACATAACCCCCAGTACCACATGCCACACGTACCACATCTTACATCCCACACACCATGCACCACATATCACACCATACATGCTTCCTTTGTG
TACCACATATATAACATCACACATACCACATACACACTCCATACCATATATCATGCTACACATACATGTACACACTACGTACTCAGTCCATAAA
CACTGCACACATCACACAAACCCCATGTGTAGCACACAGCCACACCACCACCACATCTCACCTATACCAAATACCTACATCACAAGTCACCCAC
ACTACAAATTGCCCACACCGTAAAAGTACCACCCAATGCCATGTGCGCACAAACCCTACAAACTTAAGACACTTACCTCAGGTGCTTTACGAAC
AGCATAAGCAAATTGCTTTTACCAAGCGGGGTTCCTAAGCAAGCACCTGTACATTTTTGGGTCCCCTCATTTAGTCCCTGTTTGGGGTATGGAT
TCGGGGGGCCCTAAGAGAGCTGAGGAATTGGGGTAGTGGGGACAATGAAAGCTGCTTCCTTTCCGGGCCTCCTGGGTTCTGGGCAGCTTCAGGA
TCTGGGCTCAAAGAAACTTTTGGGGCTCCTTTCTCCTAATGGGGGCCTCTGTGGTGGCCAGGTGGGTGGGGCTGGGAAAAGGGAGTCCCTGGTA
CCTCCCTTTCTCAAGGAGGGCAGGGGCACACCAGAAAGTGGTTTCCCATATCCATGACCTTTAAGAGTGTCCTCAACCATTGGGGCACTTCCTC
ATTCTGGTTCTTCAGGGGGTGGGTGTGTTAAAGATCACATGACCCTTATAGTGAGATGCAGAACTGGGTGGGGTGTGCCTAGGGTTCCTGCTGC
TCCAATTTAGGAATCCATCATAAATGCAGACAATGATGTCTTGAGAGCATCAGCCTCATCCTTGAGCAGTATCTTTAGTCCCCCCCAGAGTCCA
GAGCCCAGCTCCCTCATTCCCAGGGCCTGTTTCAGTTCACAGTCCTAAGAAGCCACTGATGGCTACATCGTCATCTCTCAAGACTTCCAGGTGC
AGTCCCGATTACCTGCATGATGAGGTGCCTGGGGGCAGAGACAGAGAAAGGGCCTCAAGCTTAGCCTCCCCTCACAGGTAACAGACATGCTTGT
ACTTGGTCTAAATTCCAGGCTGGCCAGGGCCAAATAAGATGCCATCTGAACTAGCAGCTGTCTGGACTAATGGCATGTGACTGATGGGGGAACT
CATGAAATCTGAGGCTACGGTATGAGAGAAGTTTCTAGAAGTTGAGGGAAGCTTTGAAAGAGCCCTTGTGGCCAGAGTGCAGACTTGTGGGGAC
TGTGCAGCCCATGGGGATAGGTGGGTCAGAATCCTTCCCAGGAGGCTTAGTAGTCACTAACTTGATGTCAGGAAGAGCCCAGCCCGTGTCCATG
ACTAAATACCTGACAAAAGCGAGTCAAGGGGAGGTTTTAATTATGGTTCACAGTCTGAGTCTATGAGGAATGCAGCAGGGGCATGCACCTATAG
TCAGGAAGCAGTGTTGCTCAGTTTGTGTTTCAACATCCCCTCCTCTGGGGGCATCACTGTGTAGCCTTGGCTGGCCTGGAATTTTCTAGACTAC
CACGTCTTCCAGTTCCCAGAAATCCTCCTGACTCTGCTTCCTGAATGCTAGGATTAACGGTGTGTGCCAGCCACCATGGCCAACCTCACAGGCA
TGCCTAGAGGCACATCTCCTAGGTGATTTTAGAGTGTCTTGAGCTGACAATATCTACCATCACATGAAGGTATTTGAGGCAGGTGGCACAAGAC
AGAAACAACTGTTAGCTTCAGTGAACCCCTAGCTAGGACATGGGTGAAAGAACTCTGGGAAACCTGTGGATGGGACGTGCCTGGGCAACTGTGA
TGCATAGGTCCAGTTCTGGGTCCGCCACCCAGTGCCAGATGGCACAGTACTGAGGGGGTGGCACCGTCCACTGCTTTTAATGCTCCTTCTCCCT
CTCTTTGATTTCTGCACTCAGAAGGAATTGGATAGTTAAAGAACCAACAGAGAGTAAGTACCTCTTCACCTCTCTCATGTTTTACAGTGATGCC
ACATTTAAGTTTACAGGTGCTGTGGTGTGGGGTTTGTGGCCAATGTGACCTACATGTTTTCACCCCCCACCACAAACTTGATCTACAAACAAAC
AACAAAAGCCCGGAGTTAGTGTTTCATGTTCATTCCTTGGTTGTAATACCTCAAGAGTTAGTTGTTCCAGTTGGATTGTAGATTGATACCCAAC
TGCAGAGGAATGGCTAAAACAATATGGTTCCATGAAAGGCACTGAGTTGCAAAGGAAATGCTTGGCCCTAAGATCTACAGGTAACACCCAAGAT
CCTACCAGCTCTGAATCCAAATCCACAAAGCTGCTCCTGTCCTGGATCTACACAGGCTCTGTTGTGTCATTGTTCCTTAAACTGTTTCACAGCT
GTTTAAGTAGTGTCGGCATAGTATTAAGTACTGTAAGAAATGTAGAAATGATTCCAAGTCTCCAGGAGGAGGTACATGTAGGTTTTCTGCAAAC
ATGTCATTTGATGTCCTCAGGGATCCCCTGCAGATACTAAGGGATGACTCCAGTTGTGCTCCCCAGTTGATGGACATCTGTATACAGATGTGTG
TGCGTGCATGTGGATGCGTTCATATACACCTAAATACATGGAGTTGTCGGCGGCTAGAATTGAAAACACTTGAGTAGAAAGCAAATTTAAGTCC
GCAGGGCTTGTCCACTAGTCTGCAGCCAGGTGGGTAGGCACAGGTGCTTCCGGAACACGAGACTGGCATGTTCCTGTCTTATGTATGTTTGTTT
ACTTTTTAATTTTTTAAGTTAATTTATTTTAGAGACAGAACCTTAGCATGGATCTCTGGCTAGCCTGGAGTTATGAGACTTACTCTATAGCTCA
GGTTGGCCTTGAACTTATGGTGGTTAACCCTCTGTCTCTGCCTGCTGAGTACCGGGACTACAGGTGTGCTTCATCGTGCCCACTCAATTCTGTC
TCAGTGGCATTTTTCAATTATAAAAGTAACACTTATTTATATTTGTTATTCATAAGCTCTTGGAAATTTATTTTTAAAAATTCAAGGGTCTGGA
GAGATGCATGGGTAGTTAAGAGCACTTGTTCTTGGAAAGGACATGGGTTTGATTCCCAGCACCCGAATTGTGGCTCAGGATCCCTGCAGCTCCA
GTTCCAGAGCATGCCATGCTGCCTCCTCATGCACGTGGTGCATTGGTAACAAGCCAGCAAAACACCCATACACATACAATAAAACCCATCCCCA
GGCTGGGAGGTATTTTAGTGGACAAAGCACATGCTGTGCGGCATGTACGTAATGCCTGGCAGGCAGTTGCAGTAGCCTACCTATAATCCCAGCA
CTCCCAAGGTGGGAAAGGGAGATCCTTGGAGCATGCTGGCTAGCCAGTCTGGCTGATTCAATGAGCTCATAGTATACTTGAGACCCTGCTTCAA
TATATATATATATACACAGTGCCAAGCAATAGGCGAAGACACATGGTTGACATCCATCTCCAAGCCCCACATGTATGAGTTGCATTCTGTTAAC
CCTCTGGAGCCTCACCAAGCACTGTATGACCTACATGTTTTTCAGTTTGCTTTTTTCTGAAATCAGTTTCTCTGAGAGGGTTCTCCTGACTGTC
AGTCACTGTAGTGGCTTCCTACCATTTGTATGTGTCGTCGTCGTCCCCACCCCCGCCCCCCGGACATTTCCTGATTGTTACTAACAGAAACAAG
CCTGTGGCCCTCCAGTTGCATTACCGCTTTGGAGCCGTCCCTGGGATGGTCCAGTTGTATTTCTACTTGATCCAGTGTGAGAGGAAAGCCTGGG
ACAACAGCACAGGGGGTCATTGATGAGGGAACCAGATGATGATGAATAGAGGGTTGGTGAAGTGAGGAGACTCCAGGCCCAAAGTTATAAACTA
AAAATCTCTTCAAAAGTCCAGTCTGTGGGGCTGGCATGCTGTTGGTAGAATGGTCACTTAGTATGCATGAAGTCCTAGGTTAGATCCTCAGTAC
TCCATCAACTTGTTCAGGGGTGCAGGCCTAGAACCCCAGCACGTGGGAGGTGAAGGCAGGAGGATCACAAGCGCAAGGTCATCTTTGGCTACAT
AAAGTTCAAGGATAGCTTGGGCTACACTGAGGTCCTGTCTTAGAAAAGAAAAAATGCAACCTGTCACCTGTAACTGAGTAGTGCTAACTCCCTG
CCTGGTACCATTCCTTCCCCAGGGCAAGTTCTCCCACCCTTGGCCACCCACACACCCTGGCATCATGGCGGGGACAATGCCAGGTGATTTTAAG
AGTTCCCAGGTTCTGCCTTGGTGGTTGTTATTTGTTGTTATTTTTTGTTGTTTTTCCTGCTCAGTGTTTTGAGCCCAGGTGGAGATTGCTATTT
CTTTTCTCCCCGTTTGAATGTCTCCCATCAAGCGTGCGTGTGTGCGTGTGTGTGTGTGTGTAGATGGCATGTGAAGGTGGCAGGACCTGGCTGG
CTGGTCCTTCCTGTCCAGCTCAGCTTGGTACATCACATACCTGGTAACAGTAGCTGACAAAATGTAGCACCACGAGGCTGGTTTAAACCATTGG
GCCATCTTCAGCCAAGCCCCAGGTAGGGCCAGGGAGTGAACCATTTATGAGTGTAGCTGCTTTGTTTTGCCTTCTCCTTGGTGGATGGAGGGCA
GCCTGCCCTTGCATGGCCTGAAGCTCTGGGGATCTAGGGTGAAGTCTGAGACCCAGACTATACATACCCATAAGTCAGCACTAACAGGGAAGTG
ACCTGTTGCCATCACAGC
MOUSE SEGUENCE—mRNA
GAATTCGGCACGAGGCCATCCATGTGTGCCCGTTGCTGAAGGCCTCGGTGGCCCCTGCCCATGAGGATGTGTGCCCCCGTTAGGGGGCTGTTCT
TGGCCCTGGTGGTAGTGTTGAGAACCGCGGTGGCATTTGCACCTGTGCCTCGGCTCACCTGGGAACATGGAGAGGTAGGTCTGGTGCAGTTTCA
CAAGCCAGGCATCTTTAACTACTCGGCCTTGCTGATGAGTGAGGACAAAGACACTCTGTATGTAGGCGCCCGGGAAGCAGTCTTTGCAGTGAAT
GCGCTGAACATCTCTGAGAAGCAACATGAGGTATATTGGAAGGTCTCTGAAGACAAAAAATCCAAGTGTGCAGAGAAGGGGAAATCAAAGCAGA
CGGAATGCCTAAACTACATTCGAGTACTACAGCCACTAAGCAGCACTTCCCTCTATGTGTGTGGGACCAATGCGTTCCAGCCCACCTGTGACCA
CCTGAACTTGACATCCTTCAAGTTTCTGGGGAAAAGTGAAGATGGCAAAGGAAGATGCCCCTTCGACCCCGCCCACAGCTACACATCAGTCATG
GTTGGGGGCGAGCTCTACTCTGGGACGTCCTATAATTTCTTGGGCAGTGAACCCATCATCTCTCGAAACTCTTCCCACAGTCCCTTGAGGACGG
AGTATGCCATCCCGTGGCTGAACGAGCCTAGCTTCGTCTTTGCTGACGTGATCCAGAAAAGCCCAGATGGTCCGGAGGGTGAAGATGACAAGGT
CTACTTCTTTTTTACGGAGGTATCCGTGGAGTACGAATTCGTCTTCAAGTTGATGATCCCGCGAGTTGCCAGGGTGTGCAAGGGCCACCAGGGC
GGCCTGCGGACTTTGCAAAAAAAGTGGACCTCCTTCCTAAAGGCCAGGCTGATCTGCTCCAAGCCAGACAGTGGCCTGGTCTTCAACATACTTC
AGGATGTGTTTGTGCTGAGGGCCCCGGGCCTCAAGGAGCCTGTGTTCTATGCGGTCTTCACCCCACAGCTGAACAATGTGGGTCTGTCAGCGGT
GTGCGCCTACACACTGGCCACGGTGGAGGCAGTCTTCTCCCGTGGAAAGTACATGCAGAGTGCCACAGTGGAGCAGTCTCACACCAAGTGGGTG
CGCTACAATGGCCCAGTGCCCACTCCCCGACCTGGAGCGTGTATCGACAGTGAGGCCCGGGCAGCCAACTACACCAGCTCCTTGAATCTCCCAG
ACAAAACACTGCAGTTTGTAAAAGACCACCCTTTGATGGATGACTCAGTGACCCCGATAGACAACAGACCCAAGCTGATCAAAAAAGATGTAAA
CTACACCCAGATAGTGGTAGACAGGACCCAGGCCCTGGATGGGACTTTCTACGACGTCATGTTCATCAGCACAGACCGGGGAGCTCTGCATAAA
GCAGTCATCCTTACAAAAGAGGTGCATGTCATCGAGGAGACCCAACTCTTCCGGGACTTTGAACCGGTCCTAACTCTGCTGCTATCGTCAAAGA
AGGGGAGGAAGTTTGTCTATGCAGGCTCCAACTCTGGAGTGGTCCAAGCGCCCCTGGCATTCTGCGAAAAGCACGGTAGCTGTGAAGACTGTGT
GTTAGCACGGGACCCCTACTGTGCCTGGAGCCCAGCCATCAAGGCCTGTGTTACCCTGCACCAGGAAGAGGCCTCCAGCAGGGGCTGGATTCAG
GACATGAGCGGTGACACATCCTCATGCCTGGATAAGAGTAAAGAAAGTTTCAACCAGCATTTTTTCAAGCACGGCGGCACAGCGGAACTCAAAT
GTTTCCAAAAGTCCAACCTAGCCCGGGTGGTATGGAAGTTCCAGAATGGCGAGTTGAAGGCCGCAAGTCCCAAGTACGGCTTTGTGGGCAGGAA
GCACCTGCTCATCTTCAACCTGTCGGACGGAGACAGCGGCGTGTACCAGTGCCTGTCAGAGGAAAGGGTGAGGAATAAAACGGTCTCCCAGCTG
CTGGCCAAGCACGTTCTGGAAGTGAAGATGGTACCTCGGACCCCCCCCTCACCTACCTCAGAGGATGTTCAGACAGAAGGTAGTAAGATCACAT
CCAAAATGCCGGTTGGATCTACCCAGGGGTCCTCTCCCCCTACCCCGGCTCTGTGGGCAACCTCCCCCAGAGCCGCCACCCTACCTCCCAAGTC
CTCCTCCGGCACATCCTGTGAACCAAAGATGGTCATCAACACGGTCCCCCAGCTCCACTCAGAGAAGACGGTGTATCTCAAGTCCAGTGACAAC
CGCCTGCTCATGTCTCTCCTCCTCTTCATCTTTGTCCTCTTCCTCTGCCTCTTTTCCTACAACTGCTACAAGGGCTACCTGCCCGGACAGTGCT
TAAAATTCCGCTCAGCCCTGCTGCTTGGAAAGAAAACACCCAAGTCAGACTTCTCTGACCTGGAGCAGAGTGTGAAGGAGACACTGGTCGAGCC
TGGGAGCTTCTCCCAGCAGAACGGCGACCACCCCAAGCCAGCCCTGGATACGGGCTATGAAACGGAGCAGGACACCATCACCAGCAAAGTCCCC
ACGGATCGTGAGGACTCGCAACGGATCGATGAACTCTCTGCCCGGGACAAACCGTTTGATGTCAAGTGTGAACTGAAGTTTGCAGATTCGGATG
CTGACGGGGACTGAGGCCAGCGTGTCCCAGCCCATGCCCCTCTGTCTTCGTGGAGAGTGTTGTGTTGAGCCCATTCAGTAGCCGAGTCTTGTCA
CTCTGTGCCAGCCTCAGTCCTGTGTCCCCTTTTTCTCTGGTTT
MOUSE SEQUENCE—CODING
ATGAGGATGTGTGCCCCCGTTAGGGGGCTGTTCTTGGCCCTGGTGGTAGTGTTGAGAACCGCGGTGGCATTTGCACCTGTGCCTCGGCTCACCT
GGGAACATGGAGAGGTAGGTCTGGTGCAGTTTCACAAGCCAGGCATCTTTAACTACTCGGCCTTGCTGATGAGTGAGGACAAAGACACTCTGTA
TGTAGGCGCCCGGGAAGCAGTCTTTGCAGTGAATGCGCTGAACATCTCTGAGAAGCAACATGAGGTATATTGGAAGGTCTCTGAAGACAAAAAA
TCCAAGTGTGCAGAGAAGGGGAAATCAAAGCAGACGGAATGCCTAAACTACATTCGAGTACTACAGCCACTAAGCAGCACTTCCCTCTATGTGT
GTGGGACCAATGCGTTCCAGCCCACCTGTGACCACCTGAACTTGACATCCTTCAAGTTTCTGGGGAAAAGTGAAGATGGCAAAGGAAGATGCCC
CTTCGACCCCGCCCACAGCTACACATCAGTCATGGTTGGGGGCGAGCTCTACTCTGGGACGTCCTATAATTTCTTGGGCAGTGAACCCATCATC
TCTCGAAACTCTTCCCACAGTCCCTTGAGGACGGAGTATGCCATCCCGTGGCTGAACGAGCCTAGCTTCGTCTTTGCTGACGTGATCCAGAAAA
GCCCAGATGGTCCGGAGGGTGAAGATGACAAGGTCTACTTCTTTTTTACGGAGGTATCCGTGGAGTACGAATTCGTCTTCAAGTTGATGATCCC
GCGAGTTGCCAGGGTGTGCAAGGGCGACCAGGGCGGCCTGCGGACTTTGCAAAAAAAGTGGACCTCCTTCCTAAAGCCCAGGCTGATCTGCTCC
AAGCCAGACAGTGGCCTGGTCTTCAACATACTTCAGGATGTGTTTGTGCTGAGGGCCCCGGGCCTCAAGGAGCCTGTGTTCTATGCGGTCTTCA
CCCCACAGCTGAACAATGTGGGTCTGTCAGCGGTGTGCGCCTACACACTGGCCACGGTGGAGGCAGTCTTCTCCCGTGGAAAGTACATGCAGAG
TGCCACAGTGGAGCAGTCTCACACCAAGTGGGTGCGCTACAATCGCCCAGTGCCCACTCCCCGACCTGGAGCGTGTATCGACAGTGAGGCCCGG
GCAGCCAACTACACCAGCTCCTTGAATCTCCCAGACAAAACACTGCAGTTTGTAAAAGACCACCCTTTGATGGATGACTCAGTGACCCCGATAG
ACAACAGACCCAAGCTGATCAAAAAAGATGTAAACTACACCCAGATAGTGGTAGACAGGACCCAGGCCCTGGATGGGACTTTCTACGACGTCAT
GTTCATCAGCACAGACCGGGGAGCTCTGCATAAAGCAGTCATCCTTACAAAAGAGGTGCATGTCATCGAGGAGACCCAACTCTTCCGGGACTTT
GAACCGGTCCTAACTCTGCTGCTATCGTCAAAGAAGGGGAGGAAGTTTGTCTATGCAGGCTCCAACTCTGGAGTGGTCCAAGCGCCCCTGGCAT
TCTGCGAAAAGCACGGTAGCTGTGAAGACTGTGTGTTAGCACGGGACCCCTACTGTGCCTGGAGCCCAGCCATCAAGGCCTGTGTTACCCTGCA
CCAGGAAGAGGCCTCCAGCAGGGGCTGGATTCAGGACATGAGCGGTGACACATCCTCATGCCTGGATAAGAGTAAAGAAAGTTTCAACCAGCAT
TTTTTCAAGCACGGCGGCACAGCGGAACTCAAATGTTTCCAAAAGTCCAACCTAGCCCGGGTGGTATGGAAGTTCCAGAATGGCGAGTTGAAGG
CCGCAAGTCCCAAGTACGGCTTTGTGGGCAGGAAGCACCTGCTCATCTTCAACCTGTCGGACGGAGACAGCGGCGTGTACCAGTGCCTGTCAGA
GGAAAGGGTGAGGAATAAAACGGTCTCCCAGCTGCTGGCCAAGCACGTTCTGGAAGTGAAGATGGTACCTCGGACCCCCCCCTCACCTACCTCA
GAGGATGTTCAGACAGAAGGTAGTAAGATCACATCCAAAATGCCGGTTGGATCTACCCAGGGGTCCTCTCCCCCTACCCCGGCTCTGTGGGCAA
CCTCCCCCAGAGCCGCCACCCTACCTCCCAAGTCCTCCTCCGGCACATCCTGTGAACCAAAGATGGTCATCAACACGGTCCCCCAGCTCCACTC
AGAGAAGACGGTGTATCTCAAGTCCAGTGACAACCGCCTGCTCATGTCTCTCCTCCTCTTCATCTTTGTCCTCTTCCTCTGCCTCTTTTCCTAC
AACTGCTACAAGGGCTACCTGCCCGGACAGTGCTTAAAATTCCGCTCAGCCCTGCTGCTTGGAAAGAAAACACCCAAGTCAGACTTCTCTGACC
TGGAGCAGAGTGTGAAGGAGACACTGGTCGAGCCTGGGAGCTTCTCCCAGCAGAACGGCGACCACCCCAAGCCAGCCCTGGATACGGGCTATGA
AACGGAGCAGGACACCATCACCAGCAAAGTCCCCACGGATCGTGAGGACTCGCAACGGATCGATGAACTCTCTGCCCGGGACAAACCGTTTGAT
GTCAAGTGTGAACTGAAGTTTGCAGATTCGGATGCTGACGGGGACTGA
HUMAN SEQUENCE—GENOMIC
CCTGACCCAGTCACCTCCCAAAGGCTCCCTCTCCACAAACCATCACATTGGTGGTTAGGATTTCAACATCTGAATTTGGGGGTAATCCAGACAT
TCAGACCATAGTATCACCTTTGAAAGACATGACACCAGGCATCCCTTTAGGGCCGCAGGGTTAACACAGTCCTCCCTGAGGGGCTGGCCAGCCA
CTTCCCAGAACAACCCCACACTGTGCCTGGGAGCCAGTGTCCAGTAGGGAGACCGTTGGCCCCCTTAGAGGTGGCAGCAGGTCAACATTAGGCC
AGTGGAAGATGATTATTTTTGCTAGCACTAACCTGCTTGGGCCCAAGGATGCATGCACACACCACACACCCTCCAGAAAAGACAGTATTGTCTG
TATCCAAGTCTGAAGAATAGAGTGATGTCACACAAATGGCATGACAGTCTGCATCTCTACCATGCTGGCTGCTGCTTAGAGGGGTCCCCACTCT
TCCTCTGAAGGGCCCTGTTCGTGCCGCCACTCTGCGTGCATCCTCATAGCGCTTGTCCTGCCGACATTGCTGATAGATGGATGCCTGTGTTCTT
CACTCCCACCGCCCTCTGTCTCTCTCCCTGGGTTTCCCATTCTGTACATGCTGCACCCAGGGGTTTGGGAAGTGGGTGAGGGTTAGAGCATGTC
TCTGAGATATCCTGGCTGTGGCTCGCTTGCCCCTGCAGGGATGTGGCTTGAGGGTGAAGTAGACTGGTCCTGGCAGCTGCCCTGCTGGGTGAGT
CCGGCCCAACACAGGTCCAGGGCTAGGCATGTCCCTGCAGAGCTGGAGTGTTGGTCTCCAGGGATCCATTTTAATTCTAGGAAAACTGAAGTTG
GAACAATAAACTGTTTATTATGCTTGTGACCTAGACAGCTCCTGAAACCCATTCCTGATTCCTGATGGGATAAGGCCATTTTATCCTCAGTTTG
CAAGGCAGTGACATCAGCATCCTAACTGGAGGTGGGAGAGGGGTTTCTGGGGGCAGAGGACATCCCAGGGTGGGGATACTAAGGTGGCCAGGTA
CTGGCAGGCCATGGTTGGTGAGGGCTGCGGTGTCTTCTGTTTCCCAGCACCTGGCTGCCTGCTTCCAGCTGGGAGGGAAGATGTGGTGAGTGCG
TGTCCTCCACTGTCCTGGTTGTAGGGCCTGGAGAGACTCTAACCTTTACCTGCCCCATCTGGGCAAACCCCTGAGATAAGAAATAAGCCAGAAC
ACGCTCCCCCAGGCAGTAGTTTGCATGCAGTGAGGTCAGGGGTCCAGGTAATGACTGCCTATGTTAGATTGGCCAGTCCACACCTGTCACCAGA
GATCCCCTTTCAGTTTCCCTGGGATGCCAGCCACGGCATTTTAATGCCAGTGTTGCATCCTGTTGCTGGCAGAAGCACTGACACCTTCCATGTG
GTAGTGACTGGTGTCCCCTCTGTAATCTACCCTAGCTGCCAGACAGCTGATCCCAGGAAGCAGCCGAaACACGGGTGACTGGACACTCCCCAGA
TGGCATTTTGGTTCATGGTTCCTGCCTTACAGGACACTTGCCAGCAGTCACTATTCACATCTAAGATGCAACACTAGATCAGAATGTATGTTCT
GCAACAGAGTGACACATGCACAGATGCACATATACACACATGCACACACAGATGCGCACATACATGTCTGCGTACACATACACAGATGCGCACA
CAGATGCATGCACGTATGTGCACATATAGCTGTCCACACATACATGTATATGCATACACAGATACATATGTATGGATATATGTACACAGGCAGA
CATGCACAGGTGCACACATACATTATACACACACATACACAGATCCCCATGCATACACATATGTATACACATGCAGACATCCAAGCAGATGCAC
ATGCACTCACACAGGTGTACATATGTATGCACACAGACATACACATGCATGTGCAGACATGCAGACATACACATGTACACAACACACCTGTACA
GATCTGTACAAATGTACAAACATCGATAGCTGCACATATCTATGCGGGTTCATCTGCACACATACAGAAATACAGATGCACATGCGTCCACATA
CACAGATGCATACACACAGATGTACATGTGTCCACATATATATATACATACACGCAGACATGTATGCACAGTGGTTGCTGCGTACTCCCATTTT
CTCTGCTGTCATCTCCTGCCTTCATTTGAAGACCCCTGGGGGTGGGACAGCTGCCTTCTGACAAGTACTGACAGGCAAAAGAGTGTGGGGCTCA
GAGCCCCCTTGGAGTTCCATTCTGCTGACATTTAAAGTGGGCTCGTGCACCCTCATGAACAGCCCCAATTACGTGTAAGTAATGAGAGTGACAT
TTCACAACCCTGTCTTTCTAATGGGCTCCTGTGTCAAGTCATTCTTTCTGAATGCAAACGGCAGCAATTTCACACTGTCAGGGCAAAATGCACA
TGCATACAGCTCAGCTCCCAGCGGGATACCTTTGCTTCTCAGAAATGGCGAAGAAAGCCAGCAAAATGGCCTTTTTCTCCTTTTGTTTTAATTT
AACTATTTAGAGTGAGGTCCTTAATGTAACACCCATCAATGATTATGGAGCCACTGTTCTGAAAGAACTCGCAGGGCCGGCAGATCTGTGTGAG
TTGTGGTGTGTGCTTTGCTTAAAGCAACTTTAAAATTATATGAAACAATGCATTCTTGTTGAAAAAGGAAAAAAAAACCCTCCAGTGTCACAAA
GAAAATGTGCAGCCCCCTCCCCCTCCCTGGGAGGAGCAGTATGTGGCATGGGGTCTGTCCTGACCTTTCGCTACTCATGTGGACTGTTTTAAAG
GCCATTTGAGAAGAGCAGCAGGGCCTGATTTATGCCATCGAGAATCTGCTGTCTGCTGATGAGATCAATACACTGCAGACCTGGATATAAATCC
GTGCCAGGAAGCCTGGTGGTCAATCATTGTTCTCAGGGCAGGGCCTGGAAGCCACGTCGGGGTGCCCGTGCCCTGCTGAGCTCTACATGGCAGT
ATCTGGATTGCTCCAAGGTAAAAGCAATTAAGAAAAAGGTTTCTTTTCAACATAACCAGTTTGAGTAACCGCATAAACAGCACCAGTCCTGACA
GTCCTTCGTAACCATTTAAAGCCACACAGGAGAACCTGCTGGCTTCCACCTGGGCACCTGCTGACACTCCTGGAAATACCTTCTCTCAGCCACA
TTCGTTTGGGGATGGCTTTGCCATCCCTGGGTTTGGTCGTGGATACTATTACAAGATTTGTTTTCAATCTCTCTTCACTGGCGATTTTGTTTTG
TGGAACTTTCTACTCTTGGCTCCTTACCTTTGTCCTCTCCGGGTCTGGCCCTAGTCTTCTCACTGGGATGTTTGTGTTTCTCCTGTTCTACCTC
AACTAAGTCAGGGAGGCGTTTTTAGGTCCTTAGGCCAAGTGCTATGAAAACAATACCACAGAGGGGGATGGAGTGTGGGGGTCAGGGGAGGCAA
AGGGTGACTCTGGGATGGAGCCAGCCACCAGCAGCTCCTGGGGGTAGAACAGTCTGCAAGAGGGAGCAGCAAGTGCCAAGCCTGCAAGGCAGCC
TGAGACGGGTGGGGTGCATCGGAGGGAGGGGGCAGGAGTGGAGTGGAAGAGGCTGGCCACATTAGGTGCTGGCATTCTGCATTAAATCCTGTGC
CACTGCCCAGCACTTCGGCTTTGCGGTGAACAATCCTATTTTTACAGCGTGTTTGAGCCCCCAGATCTGGCTGCCCTAGGGACCCAGGCTCTGC
ACAGAGTCAGCTCACTCTTCCCGTCCTTGGGAGCCTCCCTTCAGGGGGTCGTGTCTTCTGCTTCTGGATCCACTGGCCTGCTTTCCCCAGGCTG
TGAGGAACCAGAACATGGATGTCTTTTTAAGGTTAATTCATTTTATGAGAGTAATTAATGCAGGTAATTTTAAAGTGAAGTAGTTCTTCTTTAA
CTTGAGAGAGGAAGCAGCACTTGCTTGTCTTGTCCCTTTGTCCCTTCCCTGCTGCCCTGAGACACTGAGTTTCAACTATTTTCACTATTTTGGT
ATTTTCCTTCAAATTAGATATGCCTACATTTCCTGACTTTCCCATCTTAGATGTTGTGTTATGATGGAAGACAGGATTGTGTTCTCTGACTCTT
GTCCCCTCCCTTTCCCTCTCTTTCTTCCCCCAGACCTCCAGTGGAGTTAGTTGTAATTTTTGTAATGGGGGCTATGCAACTGGGCAGTGATAAG
TACACTCTGGTTACATTTCCATTCATGGGTCACTCTTGCTTCCCTGGGGTTAATAACTTCATTTTCTAATCTGTGTATCTGTTCTAATTCATCT
TCAGACTCTTTCCCAGAGCCTTAAAATGCATTTCTGAGAACAAAGCCACAGATGCAATCAGTAATCCACTGTGCCTTCTCCTGCCCCGCCCCAT
TCGAGCCTCTGCCTGCTCCCCAGCTCCCCGCTCTCTCTCCCTCTCCTCCACTCGCTTCCTAAGAAAGAAGACCTGGGAAGCAGCTCCCTTGTTT
TGGTGGAGCAAATCCTGCACTAGCTTTCTGAGAAAGAAGCTGAGCGAGGTAAAACATTTCAGAACTTGTAGGTTTGAAGCAGTGTTCTGTACTG
CACTCATACTGATTGATAATTTGACTAGATGGGGAATTCTACGTATGAAGTCATCTTCCCTGGGATTCAAAGGCGTTGCTTTGCTGCCTCCAGT
GGGGCACTCACAGGTAATTTCTTTGTGGCCTGTTTCTTTCTGGAAGCCTCATGTAACCCTCCTTTCAGGATGATGTGTCTGGTGTGGGCGGGTT
CCTTTCATTCACTGTGCTCCTGGCCGCTGTTATTCCGAAAGCTCAGGTCCTTCTGTTCTAGTTATTTTCTTTTTATTACTGCTTTAATAATTCC
TCATTTTTTTTCCCGGAGCTCCTGCCAGTTCATGTGGGACTCCTCAATGGATCGTCCAGTTGTCTGTTCCCCCTTTTTTCATCTCTGCTACGTG
TGTAACTTCTAGGAAACCTCTTTAGTTTACTTTCCAGCCTTTCATTTCTTTCACCACATTTTACTTTTAAGCTTGCTTCCATTTTCCACGTTGT
GTTTCTTTGTGACTTCTTCCAGGCCCTGGCTCAACTCTGGTCTGAAATTCAAATCAGTCAGCTCTGGTGGTGTGTGACAGGGTTGGTGGTAAAC
ACAGCCACTGTTAAAAATTAAATATGTGGACTTAAAATGAAACAAATTATATTTAAAAAGGTAAAATACTCAGAACTCATTCCTTTCTAGTATC
GTGTGCATTCTACTGTTATCTGTGTGGTTGCAGTTACTCGCCTCTTGTATCTGAGATGGCAGTGCCATGTGCTGGGTGCTGCTGTGCACCTCTC
CCCAGTTCAGCGGCATCATTCTGGGAGCTCGAAAGCAGCATTTACACCACAGAAATCAGCGTGTGCTGCAAGCTGGGCACCCCCCACCTTACCC
CACCGCCCACCCCCACTGCCCACCCCGTGGAGAGCCAGTTGTTACAGCTTGCCAGCACACCGTTGGTTACTGCTCACCTATTAATAGATTCCAA
AGTCACCTCCCTCCTCCATTTTTATCCTTAAAGTTCATATCCTTTTAGAGTCATTTCATTGGGTTTCCAGAGAGAGTTGAAATAAATCTCTGTG
TTCTCCCATCTCCTGGCTCTGGACTCTTAGCCTGGCCTGACCCCAGCATGCTTATTGGGATACGCAGAGGACTGATTGCAGTTGCGGGATCTAG
ATAGGAATTAGATCCCATGTAAACCATATCTCTCCAACAGTGATCCAGCATCTTTGGTATTTGACCCGAAATGAACTGGGCCTTGTGGGTCTTG
CGTCTCTGCTCCTAGCCCTCCGGTGTGGGCACTTACTTCCTTAACGTGTCCAGGCCATGAACCCTCACAGAGGCTGCTTTTGTGACTGGCAAGG
AACTCCTAGGCCTGTTCCTTGTTGTTACTGATTTTAATAGACTTTATTTTTTAGATCAACTTTAGGTTCATAGAAAAATTGAGAAGGTGGTACA
GAGATTTCCCATATGCTCCTTCCCCACACATGCACAGCCCCCTCATTTTCAGAATCCCCCCACAGAGTGGTGCCTTTGCTACAGTCGGTGAACC
CACATAGGTACCTTATCACCCGACATCTGTATCTGGACATTAGGATTGCCAGTTGGTGTTGTACATTCTGTAGGCTCGGACAAATGCATACTGA
CATGTGTCCACCGTGGTAGTATCCCTGCCAGCCTAGTTTCCCTCCCCTAAAAACCCTCTCTCTCCCCAGCCCCTGGGAACCACTGATCTTTTTA
CTGTCTTCATGGTTTTGCCTTTCCCAGAATGTCCTATAGTTGGGATCATGCAGTATGTACTTCTACAGATTGGCTTCTTTCTCTTTAATGATAA
GCTTTTAAAGTTCCTCTGTGTCTTTTCATGGCCTAATGGCTTATTTATTTTTAATGCTGAGTAACGTTCCCCTGTCTGGATGTAGCGGTTTGTA
TACCCACTCGCCTGCCCCTGGGCTGTCTCCACGGCTGGCTTCCTGTGTCGTTGGCACGCCTTACCTCTGCAGGCTCCGCATCCGTCTTCAGAGC
GCCTTCTCCCATCCCATGGCAGCTCATTGCTGTTTCTTTTGTGTGTATGGACCTTTGCCCAGCTGGGGGACATGCCACGAGAGAGGTTGTTTCT
GCTTTATCCTTGCTGTGCACACCTTGGCACTGGCTGCGACCTGGGTCTGGGTGTGGCTTGGAGGAGCCAATGTCAGTTCATTGCGTTTCACTGG
GCTGGGACTGCAGCACATGGACCAGGCCACTGGTGTCCTTGCTGCATGCATGACAAGAACCCTCTCCAAGGCCCTGCCTGCCGCAGGCCTGCCT
CCTCCTCCTCCTCCTCTAGTGGCGCGGGAGCCAGTGTGAAGTCTCTCACAATGCGAGTGTGCAGTCCTCTTCTGCTTTAAGGGATGGGGCAGCC
TGGCCACGTTGTCACTTCTGGCTCTCACTGGCTGTATGATGTTTGTCACATGGAACGGGAGAAAGGACGCATCCTTAAAATGCCAGGGGATCAT
TGCTCCAAAAGGAGGGGTAGCTGGTTTTTAATGGGAGCTTTTCTCTTGAGGGACTGTGGATAGCTTGTCACCACTGTTACCATCCTTTCTTATG
TCTGTCATTGCATGCAATGAGGACTGAGAAGAGCAAGGCCAGCAGGACCCTGGAGGGGCATGGCATGTTTGTGGGTGCCAGGCCCTGGGCTGGT
TGCTTCCACGCAGCCCCACTCCAGGGCGTGGGTTCTGTCGGGCCAGGACTTGTTTGTTCCAGGCTTAGCAGGAGCTCCATTCGTATTTGTCAGA
GCAATAAACATTATTATTATCAGATGCTAATTGATTAGTTATATATTAATATGATTTAGCAGATAATTCTCACAACTTTGTAGGCCAAGTAGGT
CAACTTCTGTTTTGCAAACAAAAAGTGACACCATTATGGCAAGGAGCTGCAAATGTGCCTGGAATTCAGAAGATCTGAACCTATAATTTATTGA
GTCTTCTTCATTGGGAGCCTATGTAGAAACCATTACTGGTTTGGGAGCTCAAAGTGGAGCAGCTTTTGGGAGCCACTCCTAATTACCAATCAGT
TATACAGAGGAAAAGCCTCTCAGATGAATGAGACTGTGCAGTGGGATTTAGACCGGCAACTTCAGAACAGACACATCTGGAAATGTTCTAAGTG
GTGTTGACAGGCAGGAGAGACAGCCGTTACACAGCCATGCAGAGGGGCCGAGCCCTGTGTGTTCAGGTGTAGCCAGGACAGTGCAGTGGAGGGA
GTGTGGATGAGGAGCAGTTGGATGGAGGTAGAGTAGGGTTGTCCATGGCCTTGCTGGCCATGATGAAAATGTGGCCCTTATTCTGAGTGATGAG
AAGGTGTTGAAACATTTCACATGGGGAAAGGATATGATCTGGATTTATGATAAGAGCATGACTTTGGCCACTCCTACTTTGTGGCAGTGGACTG
GAGGGAGGTTGTGTCAGTTTGGAATGGTATTTGTTGGTAACAAAAATCTGCAAGCAAGTAATTTAAAGATTTTAATAATTTTATTAGCAAGTCT
GCATAGACTCATAGCCTAGGGTCTCATAGGCTCTTGTGGGTTTCACTTGGCCATTCTTAGAATGAGACTTTTATCTTCATGTTGACAGCATCGC
TGCCAGAGCTCCAGCCATCACATTCTAGTTCCAGGCAGCAGCAGGAAGAGAAGGACTGAGGGAATTGCTAGCTGAATCAGCCTCCTTTAAAGAG
CTTTCCTGGAATTGCCATCTAATAAATTCCACATACAGTTTTTTTGGCCAGAACTCTCACCAGAGAAGCTGGGAGATATAGTGAGCTGGGCACA
CTGCCAACCCAAGTAACCTCGGAATTTTGTTAGCGAGGGTGCAGGAACATGGATGCTGGGGCAGGAAGCAGTGATCTCTACCATGGAAAGCAAG
AGGCCAGGATGAGAGGTGAGCATTGCTCAGATGAGTGATCGTGGTGGAGATGGACAGGAGGAGACAAGCCCCAGATGTGTTTGAGGCTGAGCAG
CAGCACCATCTATGGATTGGGGGTGGGGGTGGGGTTAGAGGTAGATGGGGTCGGCCCCTGACTGCTCGTGCTTTTTTTTAGGTGGGGACATGGA
CTGTAGGAATTGGCTGGAAGCAGAGTGAGGTTCTGTTTCGGCTGTGTCTGGCTGGGTGTGGAAGGCGGCATGTGGAGGTGGTAGGGAGGAGTGG
GGTTCTAGAGTCTGGAGCTCAGGGGTGTGGTTGTTCCATGACCCTCACAGGACTCCTGGATAGAAGGCGGAGCAAGGGACTCAGGGATCCCAGG
TTGCTGTTTTGAGTGAACTGTTTACAGAAGTGTAATGTGAAAGTGCCCAATCACTGGGTGCCCAGCTCAGGACATTCTCATAGAGTGAACCCAG
GGGTGCAGCTGGCCCCAGATCAAGGTGCTAGACGCCCTTGGGCCAGTCCTGTGGCGGGTTACGCTGTGGGGCCCAGCAAAGGTACACCAGGACA
GTAGCCATGCAGTCATCAGAGAATGTTGGGGACATGAGAGATGGGGCCAGGAGGCCTGTGCATGCCTCTCCCACCACCAGCCCCACAATTTCAC
TAAGCAGCATTAAGCCTGTTCTTTTACTAAGGGCTGTTTCTTACTGGGCAAATCAGAGTGAGTTACCACCATTTCTATGGTGCATAGACTTCTT
TAAGTGAGACTGGAAGAGCTAGCAGGGTCCCATTTGGGGCAGGACGTCAGACACTCCTGCGTGGTGAGGCCTCACGTGCGTGTGTTTCTACAGG
TGTACATCCGTGCATGTGTGTGTGTACATATGTGTGCGCATATGTTGTCTGTGTGCATGTGTGTGCACTCACATGTACATCTTAGCTCTCTCTG
CAGCCTCTGGTTTTTCTGGCCGTTTCCTCGCTGAGCGTTACAAGGCAGAGTCTCTAAGGGAAACTGAAAACCCGAGTTCTCTTCCTTCATTTTC
ATCTCATTACCAGCTGGACACAGCAGCCACACAGGAAGGAAGCTGGGCAGCCAGCCGGCCATGTGTGTCTGCCGGGTGCCCCACACCAGGTCCC
CTCCACGTGTCTGTCCTGTGTTCTGGGCTGTTAGCTGGAGGACCGCGAAGCACTGACTCCTGAAGACCCTGGCTTCGCTTTCCTGTGTCCGTTG
GGCTGTCCACTTCTAAGACTCTCTTTGTCCCCCGCTGCCTTCCGTATTCCTCAGCAGATGGACCCGGGTGAGGATCCAGGGCAAGCAGAGGGGC
TACAGAGCAGTGAGAAGCACCTCATCACTCACCATGGGTCCTGCCTTTTCTTTCTCATGTCCTTCCAGCATTTCTTCTGAGGCCCTGAAGAACT
ATCAAGTGGGCTTTGACATTGCGGTGGTGAGAGCGACCCCTCCTCACCTGGAGAACTGGCAAATGTGGATTCTCAGGGACCGCGCTGTTCACCA
GCTCCAGGCTGTGCTGCTGGCCCTGGTCCTGGGGCGCTGAGCCGCATCTGCAATAGCACACTTGCCCGGCCACCTGCTGCCGTGAGCCTTTGCT
GCTGAAGCCCCTGGGGTCGCCTCTACCTGATGAGGATGTGCACCCCCATTAGGGGGCTGCTCATGGCCCTTGCAGTGATGTTTGGGACAGCGAT
GGCATTTGCACCCATACCCCGGATCACCTGGGAGCACAGAGGTGAGTGACGAAACCTGCAGGCGTGCTGATGGCCTGGGATGGGGACCTCACTG
CCTGGGATGCTGAGGTGGGTGGCTGGGGACGGGACAGGGACCCCACTTCCTGGGATGCTGACGCGGGTGGCTGGGAACGGGACAGGGACTCCAC
TTCCTGAGATGCTGAGGTGAGTGGCTGAGGACGGGATGGGGACCCCACTTCCTGGCATGCTGAGGCGGGTGGCTGCGGATGGCATGGGGACCCC
ACTTCCTGGAATGCTGAGCCGGGTGGCTGGGGACAGGATGGGGACTCCACTTCCTGGGATGCTGAGGCGGGTGGCTGAGGACGGGATGGGGACC
CCACTTCCTGGGATGCTGAGGTGAGTGGCTGAGGACGGGACGGGGACCCCACTTCCTGGGATGCTGAGCTGGGTGGCTGGCGATGGGATGGGGA
CCCCAATTCCTGGGATGCTGAGGCGGGTGACTGGGGATGGGATGGGGACTCCACTTCCTGGGATGCTGAGGCGGGTGGCTGAGGATGGGATGGG
GACCCCACTTCCTGGGATGCTGAGGTGAGTGGCTGAGAACGGGACAGGGACCCCACTTCCTGGGATGCTGAGGTGGGTGGCTCAGAATCCGACA
GGGACCCCACTTCCTGGGATGCTGCGGTGGGTGGCTGGGGACGGGATGCGGACCCCACTTCCTGGGATGCTGAGGTGAGTGGCTGAGGTCGGGA
TGGGGACTCCACTTCCTGGGATGCTGAGGCGGGTGGCTGGGGACGGGATGGGGACTCCACTTCCTGGGATGCTGAGGCAGGTGGCTGACGATGC
TGGCATGTGATCTGTTGGATTCTCTGCTCTCAGAACTTTTGTCTGAGCAGACAGGCCTACTCAGCTGATCTAAGGGAGAGGCTGTCACAACTGT
AGATACTCAGCAAGGCTCGTGCAGACCTCCTGATTTGGTTTCTGAATCTGGGGACCCCTGACAGGAGCTTCCCTCCCTCGTACCTAGTAAATGT
TCGCGGCGTGCTGCCAAAGCACCCGCCCCACTGAGCCTCTGCTCCAGGCGCCTGAGAAGAACAGTGAGGCATCATAAGGATGCTCGTCCTACGT
GCAGCTGAGCCTCATGCCAGCTACTTTCCTTTCCTGCTGTTTAGTTTTTTTTTACTTATTTCTTCTCTTGGAGGATGTGTCTGGGGACCCCGGT
TTCTGACTCAGTGGATTGAGAGCTTACACCCTGGGTCCTGAGCAAGGTGTTGCTTGCAGGTGGCTGAGAGCTGTCAGCAATATACGCCATTCTT
GTGCTGAGAGAGAGGACCTGCTGCCTGTGTGCATCTCTCTTCCACATGGGAAAATTAGAACAGAAAGTATGAACACCTGACTCTCTTTGTTTTT
TCTCGAAGTCAGGGTCTGGCTCTGTTGCCCAGGCTAGAGTGCAGTGGGACAATCATAGCTTACTGCAGCCTCAATCTCCTGGACACAAGCAGTC
GTCCCACCTTGGCCTCCCAAAGTGCTGGGGTTACAGGCGCCCGCCACCACACCTGGCCCACCTGACTCTTAAATAAAGCAGACTCTTGGTAATA
TGTAAAAAGCCAAGGTCAAAATGCTCAAATTGTTGTCAGGACAGTTGAAGAAACAGGTCCTAAAAAAAATAATGCTGATACATTTATATTATTC
TTAAGCAGCTATTGCTCAAATCTTCATTTTCCTAAGTAGCTATTGATACAGTAACCTAATAAGCAAAGAGACGTCACTTGTTTCTCTGTCTATA
AGTTTGTGGTGGTGATCACTGAGAATTGCATTCGTTGATGGATATATTCTCCAAAAGAAATTTTGTGAAGATTTTGAAGTAAAACATTCTTTTC
CTGAATAAGAAAATCCTAAGTCATTGTATTCCTGTACACCTAATCTGTGGATGACAAGGCATGTAGACCAGAGTTGGGTGGGCAGAGGGTGGGA
GCCATCACAGGAAGCATCTGGCAGAGCAGGGTTTGGGATTCTGGGATCTGAGGGAGCAGGAAAGGGATGTCCGACTCACAGCTTCAAGTGAGCA
CACTGGCTCCGGGCTTGAGCTCTAGGGCTGTGAGTGGTTCCCATGAGCACACCTGATGGCTACACTGTGGGTCTGGCGTTGACTGAGGCCACCT
CCACTGTAGCAGTGCCTGTTGCAGCAACACATGGCCCTCGAGACATGACTGCACCCAGACGTGCTCATTGTCACTGAGACAGGGACCTAGGATG
TGCTCAGGTTTAAAAATGCTTCCATCGCTCTTTTCTTCCTCCCACTCTGACCCTGCCCTTGCATCCCACAGAGGTGCACCTGGTGCAGTTTCAT
GAGCCAGACATCTACAACTACTCAGCCTTGCTGCTGAGCGAGGACAAGGACACCTTGTACATACGTGCCCGGGAGGCGGTCTTCACTGTGAACG
CACTCAACATCTCCGAGAAGCAGCATGAGGTACGTCCTGGGCTCCCCTGCATGTCCACATAGCCCAGCTTGAGTGGGCAGCCTCTCCTGGCCCC
ACTGTGGGGCTGGGGGCTGACTTACCTGGAGCCTCCATTCTGTAGCTGTGACCATGCAGCCCCATCTTTGGGTGTCCACCAGCAGTTCACCCGG
GGACCACCCTCTTTGCTTTTTTTTTTTGAACTTTTTATTATGAAAAAATCTCAAACATACCTATGAGCACGAGACAGCTATGCCATGAGCCCTG
TGCCCACTGCAGCTTATGCAAGGCCGGGTGTTCAGTCTTGCCTACAGTACCCTCCACCTCCTGGGGCATTGAGGCAGATCCCGACACCACATTC
TTCATCTGGGTATCTCTTACTGGAAAGGGCCCCTTGTCGTGCAAATAATCAAAAATAGCTTCTCCAAAATTTATATCATCATACATTCAGCCAG
TGTTCATATTTCACCGACTCTTGTGGATGATGCTTGTTTAGTTGGTTCAAACGTTTAGTTTGAATCAGGTTGCAACCACGCTGTCCCAATCCAT
TGCAGCTGGTCACCCTGTCCATTCAGTCTCCGCTTCTAGGAGCCAGCCTCGGTTTCCTTCCTGAGAAAGCTACTCTGCACAGCCCATCCCATGT
CCCACTCTTCACATTGCTCCTGATTCCTCGGTGTCTTTAAACATTTTAAAAAATCAAACCGAATTTAAAATTTGATTGTAGCTCACTGCAGCCT
CAAACTCCTGGGCTCAAGTGATCCTCTTGCCTCAGCCTCCTGAGTACCTGGGACTGTAAGTATGTGCCACCACACCCAGCCCCACTCATGGTGT
CTTTAGCATGCACCTCTGTCTTCACATCCCCAGCTGGGTGTCAGATCCAGGCTCATCCCTCTCAGGGCTGTGAGGCGGGCTTCTCCATGGAGGC
AGGTGTTCCTCCTCCTCACTCTCGGTGGCCTTTGACCAGCGAAGTCTAGATGCTCTTCTCATCAAAGATTGCAAAACGCAATGTTCTGATTCTA
CCTCTCCTTCTCThCTTGTTAGCTGGAAATCGTATAAAAGGGGAAGTGCCCATCACCAGCTATTTTGGTCCGTAGAGGTCCCATTTCCTTTGGG
AAATGCAGAATCGATGCTTGGAATCTTTGCCTTTATTTACCAGTTTTCAGAATAATGAGTTAATGAGTCAGTTTCCCCTCTGAAGGGCAAGAGT
GAGTTTTTTGATTGATTTTTAGTGTCACAATGAAGTCATAGATTTAAAGATATTTGCTCTTCTTTGACCCATCACAGTGAAACACCTACCTCAT
GCTCAAATCGTCGCATCCATGGTCAACAGCAGCCTGTTTGGGTTGGCTCCTGAGACCTCGCGGTGTGGGGTTAGTTTTCTTGCTCTCTGGAATC
TGTTCCAGGTTCACGCTGGACATTTTCTGCCCAGATCTCTTTTTGTTTTCATCTCCAGGGTCTAGCTTATTTCTCAATTTCTGTTTGACACATA
AATGAGCTCTGAGCATTATGCTCTGAAAACAAGTATTTGGCTGGGTGCTGTGGCTCACGCATGTTATCCTAACACTGTGGGAGGTCGAGGTAGG
CGGATTACTTGAGCTCAAGAGTTTGAGACCAGCCTGGGCAACATGGCGAAACCCTTTCTCTACAAAAACTGCAAAAAATAGCCAGGTGTGGTGG
CTCACGTCTTGGGAGTCTGAGGTCCTTGGAAGTCTGAAGTGGGAGGATCACTTGAGCCCAGGAGGTCGAGGCTGCAGTGAGCTGACATCATGCC
ACTGCAGTCCAGCCTGGGCAACAGAGCGAGACCCTGTCTCAAAAAAACAAAAACAAAGTATTCAGCACTGTGAGGCACTCTGAGCAGAAAGATG
TGGTGGTCTCACCTCACTCAGCTCTTACTCTTCGCGTGTCTGTTAAATTCTCTTCCTCTGAGCGCCTTCCTGACCACACAGGCTCACACTCGGT
CTTCCGGGAATTGTGGACAGTGTGTTTGTGCTGGCCCCACAGGTGTTCAGTGAGAGCCAGACATTGCTCTGTTAATGACACCATGGTGGTCCCT
CGGGGTTGGGGAGAGGCTTGGGGACCTCTTATACTTAAAAGAAAAAAATAAAGAGACCCCCGCCAAGCATCAGTGAAAATCCCTGCAAACGTGA
TCAAGAGTCTGTTGAAGTGTACGTTCCCTTGTCCTTATGCATTTCATGCGAGTTGACGAAATCATCTGTACTTTAAATGCTGAAGGAAGTGGCT
ATTTTAATGAGACTCTGTTCCGCTTTTGTGTGCCCCTATCAGGACTTCCATTTCTATGAAATGACACTCCCTTAGTACTGTGTACTGCAGCCTG
GGTGCTGTTCAGGAACTCACAGAGTTAGTTAGAAACCTAAAGTGGGGTTCCTCTCCTCATACTCTCCCTCCTAGAAGCCCTGTGTGCTTCCTAA
CCTCTGAGTCCTGACACCTGCTACAAGCCTTTTGGTGGAGAAAAATACATTGTTACTGGTTTCTTTTGCATCAGTTATGGAACATCACAGAGAG
AGGGGGCATTTTGGGGAAGGTGAGGCAGAGATAGATATAGAAACCTTCCCTTTTGTCTTATGTTGCTTTCTATTCTGTGGGGGCTTTGGAGGGC
AAGGGGCTAAGGCCCCCACCCCCTGGAAGTTTCTGATCACGAATTGTCCCTGGTTTGAAGAAGAGGTGGTAGGAAACATGGATCTTACTAAGAT
GGTGCCTGAGCTGAGCTGGTGGTGTTACTTCATCACTTCCTCCTGCTGCCAACATGCAAGTTTAAGGAATCGTAAATCTCCCTTTCTACTGATA
CAGTGAATATTGTGGCTTTTTTTTTTAAACTGGACTGTAGAGTCACTTTTTTTTTTTTTTTTTTTTTTTTTTTTCAGACGGAGTCTTACTCTGT
TGCCCAGGCACACGAGGCTGGAGTGCAGTCCTGCGATCTCGGCTTACTGCAACCTTCGCCTCCCCGGTTCAAGAGATTCTCCTGCCTCAGCCTC
TCGAGTAGCTGGGATTACAGGCGCCCGCCACCACTCCCAGATAATTTTTGTATTTTTAGTAAAGATGGAGTTTCACCATGTTGGCCAGGCTGGT
CTGGAACTCCTGACCTCAGGTGATCCGCCTGCCTCAGCCTCCCAAAGTGCTGGGATCACAGGTGTGAGCCACTGCGCCCGGCCTGTAGAGTCAC
TTTGAATGGATGGAAAAAAGCCCACTTTGATGTTTGAAAATACTTATCGTTTAGGTTTTTGAAAAATAATTTAATTTTCTCCACTTTCACGAGT
TTGATTTTTGCTGCTGTTTTATGGTGAGTGATGGAACTTTTCGAGCTGGTCTTCCAGACTCCACTGGTTTCAGGATAAGGATCAAAGTGAGGAT
GTTCATGAAGGCCCCAGGCTGCTCCCCTGCTCTCTGAACCTCCCTTGCAGTCTCAGAATTGATCATCTCAGGACAAACCTCTAGGCTCTAAACT
CTATCATGTGCTTTTAAAATTGTCCATTTTTATACTAGTATCTCTTTAAAAGAATATGCACTCATATGGACTCTATCCCAACAGGTGTATTGGA
AGGTCTCAGAAGACAAAAAAGCAAAATGTGCAGAAAAGGGGAAATCAAAACAGGTAAATTTCTTTTGCAAGCTACAAAGAATCCCATTCAAGTA
TTTCTTGGTTGTTTTAATCTACTGAATGCCAGCATGCCTGCAGTGCAGGCCACCCTTTTCTCCAGGCTCTCTTCATTCTGCTGGGCTTGGTTTT
GTTGTGATGCGTCTTGGAGCTCATCCCTCTATTCCTGAGTATCAGGATTCGGTTTCTCAAGCAGGTGCCAAACATATTTGGCCTTTGAGAAAAT
ACTTTTTTAAAGTTTATATGAATTGAATGTATTCAGATGGCCTGAAACTTTGACTACAGAACACTGGTGAATAGTGAAGCCCACTGGTGAATAG
TGCAGAAATTCAGGGTTTGTTCTGCTTCCTTGCACCCAGAGGCCTTAGGCTTGGAGGAGTTGCTAGTCATGCAGTTTTTCTCAACCCCTTCGTT
GGACTTGCGACAGAGATGCCCCATTTATTCGGCCTGCTGTGCTCAACCTCTTGCACGAGGGAGCACATCAGTGAGCAAGTGTGGGATCTGGCCA
GCTGTTTTGGGTGCCAGCAGGAGCAGGCTTTGTGTGAGCCCTGAGGGTGGCACCCAGGTTGGGGTGCCTGTGACCCCCGCAGCCCCAGAGGGTG
CGTTACAGTGCTCTCTTATCTCTGCTCTCCATGGACAGTGGTGTGTTATAAGCTCAGTGGGCCCCTTTTCTCATTGTGTGGGACAGCTGTCCTC
TGCCAGTGAGGGCAAAGGGCCAGTATGACAGCCTTTTTTGGGTACACACACTCAGGGTCCTGAGCTCTTGTCCGGCATCCAAGAAGAATGAGGT
TGCATGGACACTTAAAGGATGGTGGAGTCAGAGAATTTTGTTTAGTGTTGGAAGTGGCTCTCAGCGGAGATGGGAGCTTGAGAGGGAACAGGAC
GAGCAAATAATCTTCCCTGAAGTCTGGCCAGCTCTGGCCGGCTCTTCTCCAAAGTTAAGCCATTTCTCCAAAATCCAGCCATCCCTCTGAAGTC
AAGTCACCTCTCTCTAGTCCAGCACCTTCTCTTCTCTATCAACTGAGTCTGGGCTCTTATAGGCACAGGATGAGGGCAGGGTGGGCTGTGGGTA
GTTTCGGAAAAGGCAACATCTGATTGTAAAAAGATGTTCAGAAAGAACCAATCGGGAGACGGCGGGCAAACAGGGATAGAAGTTCTCACTCTGG
GCTGCGGGTTTTAGGCTTTTTGGCTCACATGTGGGGTTTTGCCAGGGACCTGCCCCTGTCTGCCTAGACTTTCTCTGGCTCCTTTCTCTATCAC
TGGGACTTTTGGATTCTTGGCACACGTCCGTCCCTTTGGTCCCTCAGGCCCCTCAGTGTGGTACTCATTCAGGGAGCCCTCGGCCGAATACTCA
TTCCCGTCTGCAGCTCTAGGTTACCCTCATCACCCTCTGAGCCATCATGTCCCTACCTGGCACTGGGCATTCTTCCTCAATGGTGGTCAAGACA
AGCTGAGTTAAAGGCAAAAAAAGAAAGAAAACCCAAAGAAGTCTATGCTAAACTTTCTTAGCAAAATGCTAAACACTTGCACTGAAGTCTTTGT
CTCTAAATCCAAAGTTAGATTAGGAAATGAGAGGAAAAGCAGGTGGTGCAGTGTAGCTCCGGTTTCCGGGGGTGTGCAGGTGGGTTGGGGTGAA
TAGTGAATTACTTCCAACCAGTCTAAAAAAGAGAACTCTCCTAATCTGGGATAATTTCAGACTATAGCACATGTAACGCAAGTACTTTTAGCCT
AAAATAGTGGGTTCTGAAATGGTATCTTTGTTTTCCTGCTTGGGCCTTTCTGATGCCAAGCCGGGCTCCAGGCTCTCCTAGTGCAGCCACTCCC
ATGGGGGCTGGACTGGGACCCTGGGTTGGGTAGACCTAGTGTCGCTGTTCCTAGTTCTGTGCAAGTTGGAGGCAGGGTGGGGTCTCCAGAAACA
GAGAGGAAATTCTGTAGGGTGGACACTTCTCCAGGCCTGGCAACAGAACAAGTGGTCCACGTACTTAGCAAGAGTAAAATATATTGCTTTCAAC
AGTGGGTGTCCCAGTAAGATTTGAGGTAAGCATTCATTGAGTTTTTTTTCAGTCACCACATCTTTGCTGAGTATCAATTAGGGACTGAAACAAA
ATCCTGTTTCGCACCTGAACAGCATCTGGACACTGCAGAGACACAATTACTCAGGCCAGTTTCAGCTTTAGGTTTGCCTAGGGGTGGGGTTCGG
GTCTCAGTGAACCTTCCACTGTACGAGAACAGGCCAGTGTGTGAAGAGGAGTTGCAGAGGGTCTAGATGACAAGACAAGGCTGAGAATGCACAC
ATGCCTCGTTGCCCTGGGGCCACCTGTGACCTGGTGTTGGTGAATGTGGGTCTGTCCCCCTGAGATGAACGGCGTGAGCAGTTTGAATAGTGGA
GGGCATCTGGGCTGGACGGTTGCTAATGGTGCATTTTTCTTCCCTGCAGACAGAGTGCCTCAACTACATCCGGGTGCTGCAGCCACTCAGCGCC
ACTTCCCTTTACGTGTGTGGGACCAACGCATTCCAGCCGGCCTGTGACCACCTGGTAAGGCTGATGGGCACCTCCCTGTGCTCACGCCAGCCTG
GTCAGCAGTTAAAGTAGAAGGGGGCTCAAAGCAGACGTAAAGCTGTCTCCACCCTAAAAAAAAATAGGATTTTCTCAGAGCCAGCTGCAGAAAA
TGGGGGCAGCTTGTGGCCCCTCTGAAAGTCTTGCACAGCTGCTGTCAGGGGCACACAGCCCACTTGGCTCCTCTAAGTTTCGGTGTCCATGGCC
AAGGGTGAGGCCTGGGGTGCAGCTCAAGGTGCTGGGAGCCCTTGGTGCTAGTGCAGAGCATGCCATGGAGGGGCGTGCTGGAAAGTACAGACAG
CCTGAACATGGCATTGGGGATGTGCTGGGAAGGACAGGTAGCCTGAACATGCTATGGGGTGAGGGGACGTGCTGGAAAGGACAGACAGCCTGGC
AGCTCTGGGCATCTGGGTCAGGTGGAAAAGTCAGGACTTGCTCCTGGCTTGTGTGTTCTTGGTTGGTGCATCTGTGCTCTGGCTGTGGGACCCA
CCCTTCTGGTCGGCTGTGGTGTGCAGCTTGCTCACTGAACCACTCCTGGGTCCACCCACTCAGGGGCAGCAGGTCAAGTGTGCTGAGCACCTTG
AGAAGCAAGTCCGGCCTCTGTCCCTCACCGGCTCACATCCTTGACTGCCCAGCCTGGTATCCGGCACCTCCCCCTGTGTGACGGTCCTTTCTAT
CCAGATAATCTGATGAGCAATGTGATGATCATCAGAGAGGGAGAAGTAGCATTTTAAATAAGTCCATGTGAACATGGAAAATGAAGCACATTTT
CTACTGTGTATCCCTGGATTTTAATGAGTCCTGATAAATGTAAAATATGCCACAGTGAAAAGCATCATTTGCTAGAACCCTAAATTAATTGGAA
TTAAAATATTTTCCAATCTTGAGTCAGAGATAAATGACAAGACAACGTATGGCCTCATTAGAGACTTGATGAGGAAGCCTGGTATTTGCACACA
GACGTCTGGGGTGAGATTCATAGTAGGGGTCTACCCAGCTGCCTGTACCACAGATGCTGGCAACGAAACCTGATAGGGGGTGCGGGTCCTTTCG
GGATAGTAAGACCCTCAGTCCTCAAAGACAGTCGTTTGTATTTACTTTTGTGCAGAAAGTACGGTTGGACCAGAAACATTTTGCACAACAAAGG
ATTTTTCTGGCTAAACTCAATCAGAAGCTTAAAGTTCTGTCATTTAAACTTACATTCTGTAGATAGGTTAGCTGTTGAGGTTAGAGGATACTGT
ATTTGGTTTAAATTTAGACCTATAATTTTTCCTAATTTTTTTTTTTTTTTGAGACGCAGTTTCACTCTTGTTGCCCAGGCTGGAGTACAGTGAT
GCGATCTCGGCTCACTGCAACCTCCATCTCCTGGGTTCAAGCGATTCTCCTGCCTCAGCCTCCCGAGTAGCTGGATTACAGGCATGTGCCACCA
CGTCTGGCTAATTTTGTATTTTTAGTAGAGATGGGGTTTCTCCATGTTGGTCAGGCTGGTCTTGAACTCCTGACCTGAGGTGATCCACCCGCCT
TGGCCTCCCAAAGTGCTGGTATTACAGGCATGAGCCACCACGCCCCGTCAATTTTTCCTAATATTTTTAATAAAAGTTTTCACACCTGCAGAAA
AGATGTATCACTTGTATATCCTGGCAACCTAGATTGCACAATTGGGAGTGTGTGTGTGCACGTGTCTGTGTGCCTGTGCATGTCTATGTGTCCA
CATGTGTGTCTGTGTACATATGTGTGCTTGCTGAACTATGACCCGTGACTCCCAAATGCCTTCACAGGCATCTCCTAAGAATAGTAACATTTTC
CTATGTAACTTCAGTTTTATCATTATATCTAAGAAAACGTTCAATCATTCCCCAGCATCATCCAGTCTCCAGTCCCCGTTCAGATTTCCCCAAC
AGCCCCGAAACCTCCTTTCAAGGTATGCTAAATGGTCACATCCAGTGGGGTTCACATTCCCTAACCTAACAGGTGAAGAGCTGTGTGCTGCTTT
AACTGGCCTTTCAAGGGTGTCAGCTGCGGCCTTCCACGTGGTCTGAGCCTTCCTGCTCTGCTTCCGAGAGCGTGTTTCCAGCCAGCCTCCTGCA
GCAGAGGTGCTGGGTGCCCTTGCCCCGCCGTGGGCCCTGCCTGTCTTCAGCATGCCCAGGCTGTTGGCTGCAAAGTGGCATCCCCACGCGTGTT
CTGTGTGCTCTCTGGTTCCCGGCACGCTCAGCCCCTCTCTCGTCTGTGCTGTCCAGATGGGTTTCTTCTGTCACTTGCTGGATTTTGCCTGTTT
TTCTCCTGGATGCCCTTTCTCACTCGTTTGTCCACACTTCTCACATCTTCAGGGATGGAGACACCTCATCTGCCATTCTGTAGGCATTTCCCCC
AGTTTGTCACATATTTAACCTTGCCGTATTATTTCCCCATGTGGAACTTTCCAGTTCTCTCTGTGGTTGGATCTGTGCATTGTTTTCCTCATGT
GGCTGTGGGCACTCTGGCCAGGTAAAGAGAGGACCCCCAGATGGAAACGCCAGCCCCTGCTCTGTTTTACTACAGTGCGGTTCTGTTTTTAGTA
TTCAGATGGTTGATCCATCTGGAGTGTGTTTTGGTGGGTGGAGCGACGTAGAGATTCTGCCTGTTTATGTTTTTCTGCTTCCTATTTATTGGGT
CACCTAGCTGCTTAGAATGCCGTCTTTATTGATGTTAAAATTCTGCTCATGGGCTTCCGATTACATACTGAGCACGTCCTGTGTTGGGTTTATC
CTGGTGGCTTTCCAGCCCTAGAGCCATCAGCGTTGCCATTTCCATTTTGCAGATGAGTAAATTGACGGTCTGAGATCTTGAGAAGCTTGGCCAG
GGCAACGGAGCAAGGGCACCAAGGTGCTGATGTCTAGACCTGAGCTGAGCAACACAAGCCCTGGCCCTAGTGGGCGGGACTCCTGTAGCTCCAC
ACCTATCTGCTCGGCCCTGTTGGTCTTCCGAGAAGAGCAAATTTGTAAAATGAAGTGGTACAGAGACACATTGTTACTATAGAAATTCAGCCAT
CTGATGATGTGCTCTCTTTTTATTCAATTGTAGAACTTAACATCCTTTAAGTTTCTGGGGAAAAATGAAGATGGCAAAGGAAGATGTCCCTTTG
ACCCAGCACACAGCTACACATCCGTCATGGTTGGTGAGTCCTGCCCCTCCAGTTCCTTCACCGTGAAGATTACAGTGGCATGTTAATTACCAAA
CAGGATATTTGGATCTTCTCTGAAGTGCTGTCTTCCTTGGCTTTGTTTAAATAGGAGCTATCCTTACAAAAGGCACATGTCACTGCATAGACCC
TTTAACTTTTGAATTCATTGTTTGCAAGGATAATAGCCGCCATGGTAAACGTCACTTTGTATCCATTGCTGGCTCATGTCATTTGGATAATCCC
CGCGTGCTGAGCAGATGCTCCTGTTTGAGAGTAAGGTTTTGACTGAGCCACATGCTGTCTGCAGTGGTCTCTCACCTCTGAGCAAGTGGTCATA
GCCTGGTGTGTACCCACTGTTGCATCTGCCAGCATTTTCCTGGATGTGGGAATTCCTTGCTCATGTTTACTTATTGCCCCATGACTCTTAAAAT
GCAGCTGAACATGGAGCATGAAATTATCCCCATCAAAGTGAGAGCACACCTGTGGTCCTAGCTACTCGGGAGGCTCAGGCGGGAGGACAGGAGT
TTGAGACCAGCCTGGGAAACAGCAAGAACTCATCTCAAGCCAGGCTCAGTGGCTCATGCCTCGATCACTTGAGGTCAGGAGTTCAAGACCAGCT
GGTCAACAGGGTTAAACCCCATCTCTACCAAAAAATGTAAAAATTAGCCAGACATGGTGGCGCGCACCTCTAGTCCAAGCTGCTTGGGAGGTTG
AGGCAGGAGAATTGCTTGAACTGGGAGGTGGAGGTGGCAGTGAGCCAAGATCGCACCACTGCACTCCAGCCTGGGTGACAGAGTGAGACTCTGT
CTCAAAACAAAACAAAAAAACCTCATCTCGAAAAAGAAAGAACTAGAAGACACAGGACACCCCAAAAGTTGAGGAGACTGTCTTTCCCTTTCCC
CAGGCAGGGACTTAATTGGGTTATAAAGGAAAACACCGTGTTGAATGTTTGTCCCAGTGTTGGTGCCATAGCCTGAGAGGTTGGTTCCCTTTTC
CTCTTTTCTTCTGGGCCCCTGCAGATGGAGAACTTTATTCGGGGACGTCGTATAATTTTTTGGGAAGTGAACCCATCATCTCCCGAAATTCTTC
CCACAGTCCTCTGAGGACAGAATATGCAATCCCTTGGCTGAACGGTAAGGAAGAGCAGTGCACCTTAGTGGGAGGTGCGTGTCTCCTCATGAGT
GCACACCTGTTGATCTCTCCTTTTCTGTCTCTGAGCCCTGGGGGCCCCCGAGCTTGTGTTAGCCCCTGTTTGTGGGGGATACTTCCCAAACTGT
CCAGCAATGGCCAGAGTCCCAGGGCACCTTGTTGGGTGTTTTCACTGGTAACAGAGTACTGTTTTAAGACAGCTCCCCACTAACCCCCCAAAAA
TGGATTTGGGAGACTCTCATAAAATTGAGAATCTGATAGTAGATGGTAAACTAAGACTAATCAGATGGGGACATGGTATAATTTCCAGAATGAT
ATTAAAGTGTGGCACCAACACTGGTGTGGGACCTGCCTCCGGGCCCAGCCTCTAGTGCCCGGTCAGATGGGCTCTGGGTGCCTTGTTGCTGCCA
GCAGCACATGTCCGATGTCCGTCTTCAATGTGCAGGAAGAAGCTGGTGGCTGTTCATGGGGGCTTTTCTTGTGGGTGCCAGCCAGTAGAGCCTT
GGGTTTTGAACCTCTTGACCTTAGGGCAGATCCCAAGACCCCAGATTCAGGGACAGTCGGCCATTCCTCAGGGTGGGGCAGGATCTGGTGGTTA
GAGTTTGTTTATGTGAAGCATGTGTTGTTTAAACCAGCAAATGCATGCCTTACGTAGGTCTCTGATGCTGGTACCATCTGGCTCCCTGCCTTCC
AGTCAGGAGGCCCAGCCTCAGTTCTTGGGGTGAACCTGGGTGGGATGCACTGCAGAACCACTGTCGGGGGCACCTCTCTGAGGCCGAGGAGGTC
GGGGTAGGGCACCATCTGGGCTCAGGGCTGTCATCATCCTCAGATCCTGGAGTTTCTGTTTGGATTGTGGCATGTGCACACTCCAGACATCCTT
GCAGGGAAGCCCTCAGGCCCCCAAGGTAGTGTTGGCCGGCCTCGTGACCTCATGGGTGCAGGCAGTCCCCCAGCTCTGTGTTCTGGGATAATCA
CTGTCCTCTCTCTGCAGAGCCTAGTTTCGTGTTTGCTGACGTGATCCGAAAAAGCCCAGACAGCCCCGACGGCGAGGATGACAGGGTCTACTTC
TTCTTCACGGAGGTGTCTGTGGAGTATGAGTTTGTGTTCAGGGTGCTGATCCCACGGATAGCAAGAGTGTGCAAGGTGAGTGCCAGGCACTCTG
GCTTCGCTTGGCCCCTGCCATGGCAATAGTGTGCCTGGATGATGGGTGGCTCCTGACCTTCGTCTCAAATGTCACTTTCCCTAGCACTTTATTC
CCTTGGGCCTCAGATGCAGCTCTCAATGGATCTTATTTCTGGGGGTTGTGGGGACTGAGGGAGTGCTGGCCCCCATGTGTGACCCATTAGCCTC
CTCTTCTCAGGAGGTTCTTTCTTTTAATGCACCCTCTTCAAACCTCTAGCTTATAAAAAGCAAGGCTTTGTGGAATGTATTCACCAGAGTCTGT
TGGAATTTCACCATTTGGCTCTTAATCTACCTAAGAGTGAAACATTAACCGACAGCTTAAGATTAAGAAGAGAGAACCAATACAGAAGTTTGTC
AAAACACAAATCTTTAAGTACATTAAAAGGGGAGGGTGCCTTTAAAATGACACACTGTAGGCACAGTGCTCAGCACAAGAGAATGGCCTATGGG
CTTGATTACAGGAAATCTCAAGGTTGCTCAGTGGCAGTTTCCAGGAGGTGGATTGTCAGGCCTCAGGGGAGCTGGGGGAAACCACATGAAACAC
AGCCGCCATGTCCCTGGCCAGGAGCTCATCTCGGAGCAGGTAGCCATTCGTCTCTCATGGAAATTGGTAGATAGTTCTTGCGTGGCCCTTTCTC
ATGTAGTCATGACCATCAGGACATCAGAGAAGCAGGGTTCCTTTCATGCCTATGGCGCACAAAAGTGGGTGCCTTTGACACCGTTCCCTTAACA
GCCCCTTGCATAAGGAAGAAGGTTGGGTGATGTGCACACCTGAGTATCCACCTGGTGCTTTGCTCCCTGCCCAGGCCTTCCGTAGCCAGGGCTC
ACTGACCAGATCCTCCAGCTGCCCCCCAGCCGTGGCTGCCATGCTCCTGTCTTGCAGACCAGGAAACCATCCTATCAACAGTATCAGTGTTGGG
GGCAAGCCAGTGCCCCTTGAGATGGGGAGGGTGCTGCAGGGCCAGAGCTCCTGGCAGGCGTGCACAGTGCTCAGAATGCTGTCAGAGCAGCACT
GGCCAGGGGACTCGAGCCCTCCCCTGCCATCGGGAAGCCAAGGATCAGACTTGCCTTGACCTTGACTTATTACCCAGCACTGAGGGTTTGCAGC
AGGATCAGTGAGCAGATTCAGCGATGCACAGAGCAGCCACCTCCACCCCCGACAAGTTTCTACAAGTCTTAGGCACACTGCTGTCATCCCCTCT
GATCCCTTTTTTTTCCCCATACTTGTTTGAACTCCTGGACTAGGGTGGTTTGAGCTTAGGCCCCACAGCTCCTGCCTACCACGCAGGCACACAT
GAACCCTTCACCTGCTGCCCGTGACCTGAAAGCCACATGAGACCCCTGACGAAAAATGTGCTAGAAATTATTCTTTGCTAAAACATCAAGGAGG
AAAATGAAGTCCTTTTATCAAAAAGAGAATTTGGGCTCCTGTAATCACACAGCAGAGATGAAGGGGGAACATTTTCCCTGTGTCCAAAATAAGT
AAGCCTGCTTCTATTTCATATTCTTCTGCACTCAGGTTTTGTGGTTTCAGTATTTCTCTCCAGACCACACACAGTCATGTGTAGTTTATAATTG
TGTCTTGGTCATCGGATCCCTGAAAAGCGTTTATAAAGAACTTAGGTTCAAAGCACTTTAGCAGATACTTTGGGTAGAAAGGAAGTGAAGGATC
TATTCCTTTAAAGGGAGGAAGGTTCACACATTTGCCCAAGACAGGCTGCAGGGCCGGTGGCTGTCCCCTGGAAGGAGGCTGCCGCCACTTGCCC
CACCCAGAGCATGTTCATTGATGGGCCCTATGTCAGGAAAGTCACACAATGCCAGGGGCCTTGGCAGTTGGAAGTGTGTGAGCACGGCAGCACC
TCTGCGGTTGCAGGACTGAATGCTGGGCACAGGCAGGCATGGACGATCAAATGAGCTTTCAGTGAGAGGGCTTTTGTCCGTTTTGTTTTGTGTG
TTAACCCAGGAGCGTTCTGGGATCCAAGCTGGAACCTGGACGTGGTGCTGCGGGCCACTCACATAATTGCGGCATCTGTCTCCTGTTGCCCGAG
CTTTGTTTCAGTCAGGCACAGCCGCGCTGTGCTGGGCCATGGAGGGGTGCACCTCTGACCTTAGTGCTTGCTCACAGGGGATCCACTCTCGGCC
CACCACGTCTTCTTGTTGGGGTTTTAGGGGGACCAGGGCGGCCTGAGGACCTTGCAGAAGAAATGGACCTCCTTCCTGAAAGCCCGACTCATCT
GCTCCCGGCCAGACAGCGGCTTGGTCTTCAATGTGCTGCGGGATGTCTTCGTGCTCAGGTCCCCGGGCCTGAAGGTGCCTGTGTTCTATGCACT
CTTCACCCCACAGCTGTAAGTGCCCTGGGTGGAGTCCCCCTCCTTGCTCCCTTGATGCCGCCAGGTGGTCCCGTCACCTCTTTCCCCTCCCCAG
GAACAACGTGGGGCTGTCGGCAGTGTGCGCCTACAACCTGTCCACAGCCGAGGAGGTCTTCTCCCACGGGAAGTACATGCAGAGCACCACAGTG
GAGCAGTCCCACACCAAGTGGGTGCGCTATAATGGCCCGGTACCCAAGCCGCGGCCTGGAGCGGTGAGCTGGGGCACTGGGGGCGGGCCGTGCT
TTCCCTTCAAGGCCCAGGGCATGGAATCCAGTACAGGCACTGGGGCCTGCTCATGGGCTGGGTACCTGCCCACGCCAAGGCTCTCTGCATTGGG
ACAGGGACGGCCGATGAGCACAGCTCATCCTTCTTCCCTTTTGCATGTTACGTAACAGCCATTGCTACACTATTGCACAAACAAGAAGGCTGGG
GCAGCTGTTTTGCACAATTAACATTGCATAACAATTAGGTCTGTTTCATTTTCAGTTTCTTACACAGAAACACATGATTCCTTGGCAAAGGCCT
CTGCACTGTCCACTAGTCAACAGGAGTCACATTTTTTCCTCCTAAATATAGTGAACTTTATGTAGAAATTGGGAATTTCCAGGCCGATTCCTTC
CCTCACATGGGGAAATGGGTGTGACAGCCATGTGCACAGCCCTCTGTGGGGCTGTCACCTACATGGGGTGTCCTGGCCCTCAGAGTTCACTGCA
GCACCAGCTCCCTCCCAAGGGCAGCTGTCCCTGACACCATCATGGCTCCTTCTATGCATAGCTTCGTAAAATGTGTTCACTCATGGTCACAGAT
TTTGGTTTACACACATCAGGGAGCTAGAACAGGAAAAAACAATCCCTCAGGCTCTTATCCTATTTTAAATTCTCCTGGTTTTAGCCATGTTTAG
CATTCTTTTCATGAGAAAGCTGCAAATCAGTGTTCTGTTTTTTAATTTCAAAAGCTCAATAAGTGTTTTTCACTTACAAATAGATACAAATTCC
TTCCCTGAGTATGTATCGTGAATGCAATTCTGTCTTCCAGATGTGTTTTATAACTGTGTCCTCTGCTGGCTGGGGAGAGGCCACCTTTCCTCAT
AGTAAAGTTATTCACTGCTACCCTCATTTCAAGGCATGCATTGTGTGGTTTCCAGGTTTTGCAGCCCCCCCTGCGTTGGCTGCTTGTGTGGAAG
CACCCGTTGCGTGGTGCGACACGAGCACGTTAACAGCGGGGATTTTCTCCCTGCAGTGCATCGACAGCGAGGCACGGGCCGCCAACTACACCAG
CTCCTTGAATTTGCCAGACAAGACGCTGCAGTTCGTTAAAGACCACCCTTTGATGGATGACTCGCTAACCCCAATAGACAACAGGCCCAGGTTA
ATCAAGAAAGATGTGAACTACACCCAGATCGTGGTGGACCGGACCCAGGCCCTGGATGGGACTGTCTATGATGTCATGTTTGTCAGCACAGGTG
GGTTCCGAGGGCTTGGCAGGCTTGACAGTGACAGCACATCTGTGTCCTCTCGTAGGAAAACTGGGAAATCCAATTATTCTTTCGTGAGTAGTTC
CATTGCCTCCCTCGAGGTGACCTGGGAGGTTTGTGGGAGCGTAAGAAGATTTCTGAGAAGTCACCACACCAGGTGGCTTTCACATCTGGGACCA
CCAGGCTACTGGAGGTACTTGTTAAAAATAAAAATGCAAGTCCTGGCCTGTGGCTGGGCTGGGCTGGGGCAGAGGAGTACTGGCCGCCGAGGAG
TTGGGGCAGTGCCCACCCAGGGGGCAGGTGTGAGCCACCTGGGAGGAGGCGTCAGCCTGGCGTTTCCTGACTGCTGGCAGAGGACCACGTCCCT
GCTTCCTGGCCGAGCAGCCAGGTTTTAGATGTGCCCTCGCATGTGGACTCTGAAATAAGCACTTCTGGAACAGGGACAGGGGCCAGGAGCCACG
GTGGCTGGCAGCGCATTGTGGCCTGCCCTTCGAGCCTCCCAGCCAACCTCACATGCCCCCTGTCCTCACCGCCTGCCTAGGGCACTTACCAGTG
ACACCCCGAGGGCTGGTCGAGCATGGGAGCCCCCTCATGTGGCACTTAGCCTCAAGTGTAATGTCGACTGTTGGTGAGGATTCTTGGGGTCAAC
CATGGAACAGTTTATTTTCCTTGGAAAAAAACTGGAATAACCCATAACACTTGGGTTTCCAGTTGTCTGTTTTGCCAAAGCCTTTTCCTTTTTT
TGTTTCTAATCCGAAATCATCATTGTTTTCTAAGTATCTGTGGAGGGAATGACTTGAGAGTGAAGAGTGTGAAGTTTGCCGCTGTGGTCACTGT
CCTTGGTTTCTGTGTCTGGGTTACTGTCACTGACCTGTAGCTTTGGCCCTGCAGACCGGGGAGCTCTGCACAAAGCCATCAGCCTCGAGCACGC
TGTTCACATCATCGAGGAGACCCAGCTCTTCCAGGACTTTGAGCCAGTCCAGACCCTGCTGCTGTCTTCAAAGAAGGTAAAGTGACGCAGCCAG
CTGGACCGGGGGCTTCTCCGCTCGGTGGCTTTGATTCTAGGTGACAGGAGAGTTGGCAAGCTGGAGTCGGGCTCCTGGGCAGGCCCCTCTGTCT
GTCTGCCCTGGGTGAGCCAACCTTCAGAGTTTGACACAGAGCCATTCCGTGAGCTGTGGGATCTGAATTGGGGAAGGCAGGAAGGCAGACCTGG
GGAGACGGGACTTTCTCTTCGAAGTTGCTGGAATGTCATTGAGTTGTTGGTGCAGCTTTACAGGCTGTGGAGAGGTGGGCACTGCTCCATGAAG
ACACCTTTTTATGCCCTGAGCTTGGGGGGTGTGCAGACTGCATCTGTGGGATATGATAGTGATACAGCTGACTCGTCACCGTGGAAAATCCACA
AGAGAAGTGGTTCGTCTCATCTGTAAAGCTGTCTCCACCTGTGGCTGAGACTCTGGTCTGGCCTGCAATACTCCCAAGCAGCCCTTGGCAGGGG
TGGGTGGGCGGGCATCCACGCTGGCCTTTAGGGGGCTGCCTCCCTGGCCCTGGCTCCTAACGCGACCTCCAACCACCTTGTGGGATCCAGGCCC
AGTCAGTCCTCTCCTCCCTCAGCTGGGAGCAACAGGCAGAGGAAGAGGGCCCCTGTCTGGCCCGGTAAGATACCTACCCTTGGCAGGTGCGTAT
TTGCAAACACCCCTCCAGGTCTTCTGTCCTCTTCCCTCCCCTGTATCCACAAAGCTTGGGAGCCTGGGTCCTTTTACGTGGTCACCTCCCCTCT
GCTAATGGAGAGTTCTCCCTCAGATCTGAAAGGAAAAGAGAATGAAATAGCCTGAGCAAGTTATAACCCACTGGGTACTGGCATCTGTTCACCA
GCCTCTCGGTCTCCAGGACACAAAGGGAGCCCTGGAGATTTGAAGGGTCGACGCTTTAGCCACTGTGGGGCCTCACCCCAGCCTCGTTGTTGAA
AGAAGGGGACCCAGAAGCTCTTCTGGGGCCATCGTGCTGTTCAGTGGCCTCAGCCAGACTGGGCAGGCACCTACAACATCTCTAAAATGTGGAC
TTCTCATTAGAATGAAGATTGCCCCCCTCGTGTGGACATTCGTGAGAATGACACTCTGTTCCAAGGGTCACTTAGGGACCCAGGCTGGTGGAGG
CTTCCAGGGCACATGGGCACTGATCCTCAAAGGCAGAAGGGGGTAGGAAGAGGAGGGCCGCACAGAGGGCGTTACGGGTCAGTGCCCACCCGCC
ATCTCCCAGAACTCGGTCACATCGCCACACCAGGGCAGCTGGCCACACAGGGAGCCTCGCGCCCAGGAGGAGGAGCCAGTGCGTTTGGGGAAGC
GCTTGCTGGTCCTACTGGACCACCCGACCCGCCTGTCTCTGTAGGAGCCTCCATTCGTCGGGACGTGGCTTAGTTGCCTGGACAAGCGGAAATA
CATCCTGCCTGCCGTGGGGTCCTGTGCCTTCTCCTTGGGCCTCAGTAGGCTCCAGGTCCGGGTGCAAGGCCCAGCTCCATGGCCATGGCAGCAG
GAAGTGGCTGTTCAGTGGTAAAAAGACTGTGCCGCCTGACCCCATCAGGCTCTCTCACGGTGATCAGCAAATGGAGAAACGGCCACTACCACGC
CCTCCCCCTGTCCTACAGCTGGGGAGGTGCGCTGTGCCTCCACCCTCACCTCCTCTCCCCTGATGACAAGCATGGGGTTTTGCTTTTTATTTTG
GTAAATACACATCAGATGAAATTTGCCGTCTTAACCATTTTTCATTGTGCACGTCAGTGGCATTAAGAACATTCACGTTGTTTTGCACCCATTA
CCACTGTCTGTCTGCAGGGTGCTTTTCATCTTCCCAAACAGAAGCTTTTTACCTGTGAAATACAAACTCTTCATTTTCCCCTCCTCCAGCCCTG
GCCAGCCGCCATTCTGCTTTCTGTTTCTGTCTGGGTACCTCATATGAGTGGAATCACACAGTATTTATCCATTTGTAACTGGCTTATTTCACTT
AACATAAAGTCCTCAAGGTTCCTCCCTGCTGTAGCAGGGGTCAGGGTTTTCCAAGGCTACATACTCCTCTGTCGTATGTGAATACCGCATTTTG
TGAGTCCCGCATTTTGTGTGTGCGTTCATCCCTTGATGGACACGGTTGCTCCCACCTTCTGGCTTCTGTGAACAGTGCTGCTGTGAACGTGGGT
GTGCAGATACCTCAAGTCCTTGCTTTCCATTCCTTGGGCGCACACAGCAGATGCGGTTTGCTCGGCCCTCTCTGAGCCCACCGAGTAGGCTGTG
TCTGTATTTTCACCAGGACTCCTTGGGATGCTGAGCTACAGCTAGCGTGGCAGAGGGCAAGGAAGGCACTTAGACAGCTCAGTTGCCTCTGATC
ACGTCCACCGTCACTCCAACCTGCCCTCGGGGTCTCACGCGCACACACAGCAGCAGAGACAGAGATGGGAGCATGAGAGCTGCCTGCAAGGGGA
CAATCCTCAGGCCCAGGGAAGGCGGACACAGGGGGCGTCGAGGGGCAGGAGTGCACAGGACAGGCTTGCTCTCAGCGGGCCTCACTCATGCTGC
CAGGTGCCTGGTCAGGCTTTTTTTCTTTGCCTTTAAAAATTTTGTCTTGAAATAATTTTAAATTTACAGAAGAGTTTTGATGATTGCACAGAAG
GATCTTCAGCGCCCCCACCTAGCTCCACATGTCCGATGTCTCACATAGCCAGTGTGCATTTATCTGAGCCAAGGTGCTGTCATGGTGCAGTGCG
TTACCTGAACCCCAGACCCTGGACCCTCCTACCTCCCGCCCACCACATTCCTGGCTCAGCACCTCAGCTTGCACTGGACACTGCATCTCCTCGG
CTTCTGCTCTGTTCCTGCCTCCCTGACCTCAGCACTCCTGGAGAGGAGTAGTCTGGTGTTTTGTGGTGTGTCTGGTGTATTCTTGTGATTATCT
TGGGGTCGTGGTTTTAGGGGGAGCACCAAAGAGGGCGCTTTCTGGGGATGGGGCATCTGTATCACTCGCACCAGCGTGATGACCTTGGTCATTC
TGTCCAAGTCACCACCGTCTTTTTCCATGTCGAGTGTGTCGGGGCATGTAGTCCACACTCGGGGTGGGGGAGAGTGAGCCCCACCTCCCAGGGG
AGTCATGTGGGAAACCCCCACAGTCATTCATGCTTATTTGTAGGGACAGACTTGGAGGCTACAGAAATGCCCTGTTCTCCCACCCACTCACATC
CACATTGACCAGTGGCTCCTGCCTGCAGCAGTTACAGCTGCCTGTCCTGACGACCAGTTCCTCCTTCCCTGTCCCCTCTTCCCTTCCACAGTCA
TCCCTTGGTCTCTGTGTGGCGTCGTTTATTTTTTAAAGCTTAGTTTCATTTGGGCTCCACCCCAGCCCCATCCGGGAGAAGCGGAAGCTGGGGC
TGAGTCTCCCGCAGGGTGCAGGAGGTGGTCTGGATCCCCTTGGGACAGGCCTTGCCCCCCAGAGCCCACCCCACCCTCCACAGCTACCCTCAAG
CCTCTCTTTGGGGTGAGGAGTCAGGCGGGCCCCACTTGGCCACCCAGGGCACCCTGCTCTGCACTGCATGGTGTTCACGCCACCTCGCAGCTGC
CCCCTGCGGGACCCAACAGGACCAGCTGTCCCTTGGAAGTTACCCAGCTTGGCTTTGCTGCCAGAGAATGTTTCCGCCACCTCCACAAGCCGCG
CGATGCCCGAACTGTGATCTGGGATGTCATCCTCAATCCTTCCCACAAACGTGGCTCACACATGGCCTCATCTGCCCAGGTCTCTGCTGCTGCT
GCTGCTGTGAGGCCACCGGCGGGCCAGGTTCCCTCTGCTGTGTATTATCACAGTTTCCATCCTTGGGGGCTTTCCCTTTCTCTCGTGGGTCTGT
GTCCGCACAGCTGGATGTGTGCCCTGGGGCTTCCTGTATTGGCATCAGGGCCTGTGATCTTCCAACCACACTCTGAAGTTGACTCCGAGCGTAT
CCTCTGAGGATGAAGGTCGTGTTTCATCTGACTGTAAACCACTAAGTCATGTGTAAATTTGGGAATCTTGTATTGTTAACCAAAAGGTGTCCCC
AGGCTGTTGAGGCACATGCCCTGGGCAGAGGCTAGAGAGCGCGCTCCACGGGCCACCTCCAGGTCTGTTGCACGCCCCCGAGCTTCAGGTGGGT
GCACTGCATGTGGGGCACCCTCCTTCCAGCTGAGTTCCGTTCAAAAGCAGTCAGCAGCCCTGGATGCCTGTGTGCTGGGCACTGCTGGTGCCAG
GTGGGCTGATCCGATGGACCTGAAGCTGGTGTCACTGGGAACCAAGTGTCCCAGAACCTGGGAGAGTCTCACCTGCCCTCTGCTCTGCGTGTGG
CTGCTCTGGACCTCGGAGGTTGACTTCCCGTCTGTGAGCTCTTCCCTTTTACATATACATAAGTGAAAAGCAAAAGGCATTCTTCCTTTTTTAT
GTTTTGTATCACGATCTCCTCATCCTTTGCATGTCGTCCGTTTGGCATCCCTGTTATGTTAGGCCTCTGCTGGATCCAGACCCTCACGAGAGGC
TCCCCGCCCACAGGTGCTCACATTCTGGCTCGGGGGACGAGGTTGCGCTTCGAGGTGGTGTGCTTCAGCCTCTGGCTGCAGCCCCTTCTCACCT
ATAGTGAGGCCGGGAAGATGACCGGGAGTGCCAGGCTCATGCATCCTCTGCTGCAGGGAGGAGATCTGGAGGTGGGGACTGAACAGCCTCCCCA
CTGGCTAAGAGAAACACGTGCAGAGAAGCGCTTTGCTGGAGGGCAGGCACCTGTCGGTGCCCAGACAGGGGAGAGAGGGATTTGCACCTAAGGC
CCAGTGAGCCCTGACATAGCACGTCGATTCTGCAGGAAGGTCACAGACCTGCCTTGTTTGCCTCTGGTTCTGAGTACCTCTCCTCCTTACTGCA
ATGTCCCCTGAAGGTACACTCTGCCTGCTTCCCACACACTGGTGCAGAAGCAGAGGCCATGCTCCTGGATGCCTCCTTGCCTGATGCTAGTTCT
TCTCTGTCTCATACACGCAGGGCAACAGGTTTGTCTATGCTGGCTCTAACTCGGGCGTGGTCCAGGCCCCGCTGGCCTTCTGTGGGAAGCACGG
CACCTGCGAGGACTGTGTGCTGGCGCGGGACCCCTACTGCGCCTGGAGCCCGCCCACAGCGACCTGCGTGGCTCTGCACCAGACCGAGAGCCCC
AGCAGGTATGGGGCGGGATGCGCTGCCTGGGGATGTCCCCCATGTGTGTGGGTGAATAACACGTGCCGGTTGTTTTGCAGGGGTTTGATTCAGG
AGATGAGCGGCGATGCTTCTGTGTGCCCGGGTGAGTGACTCCTCGACGTCCCATTTCCTTTGGGAGTGGCGAGGTCTTGTAGCCATTTCACGGT
GCTTCAGTTCTGTGGCATTTGTGTGTGTTTTGTTTTTCTTTCTCCACTCGGAACAGAGGTCAAGTGTTCTGTAGCAGACTCGTGGTCTTCTCTG
TCTTTTTTTCTTGCCGAAAGAGTACTTGAAGCCACTTAGAGTGTATCTGCTTACTGGGAAGCAGTCTCCTTCCAAAACCATCCTGTTTTCTTTC
TGCTTAGCTTGGGGCTCATTAACCAAAACCTAAGCCTGTTATAAATGGGGAGAAAGGAGCCCATGAGTGATTAATCCTCAGGCAGTGTAAGGGC
TGGGGTGGTGTTTCCCCTGGGGCTCCCACCTGGGATTCACCCGCCTGTGGATGTCTGAAATAGCACCACACCCAGAGGGAGGGGTGGCATCACG
CCAGATGCCCCTTCCCCAGCAGCCACCTTCCACGGTGCTAAAGGCAGACTTGACCCTCAGTTCTGTGTCTTCCTGACCCTGACAAGACAGCTGT
CATCAGACTAAGAAAGGTTTAGGCAGCTGGGCATGGTGGTACACGCCTGTAGTCCCAGCTACTAGGGAGGCTGAGGCAGCAGGATTGCTTGTGC
CCAGGGGTTCAAGGCTCAGTGAGCTTATCATGCCACTGCACTCCAGCCTGGGGTGACAAAGTGAGACCCAGACTCTAAAAAGAAAACAAAAGCT
GGCCGGGCACAGTGGCTCATGCCTGTAATCCCAGCATTTTGGGAGTCTGAGGCAAGAGGATCACTTGAGCTCAGGAGTTCGAGACTACCCTGGG
CAACGTGGTGAGACCCTGTCTCTACAAAAAATACAAAAATTAGCCAGGCCTGGAGGTGCTCACCTGTAGTCCCAGCTACTCTGGAGACTGAGGT
GGGAAGATCCCTTGAGCTGGGGGGGTTGAAGCTGCAGTGAGCTGTGATTGCACCACTGCACTCCAGCCTGGGCAACAGAGCCAGACCCTGTTTC
AAAAAAAAGAAAAAAAAGAAAAGTTTAGGCAGGAGGAGAGTGCTGAGATGCACTGAAAGGCCAGTCTTCCTTGCAGCCATTAGCAGAAGCGTCT
CGTGTCTGATTCAAAGAGCTGACACTAGGCACAGAACTGGCCTCTGCTCAGCTTCCCCAGCCTGACGCGTTTTCTTCTGGGACACGGGTGAGAA
ATGGGATTTCTGCTGCTCAGAGCTGCCTCCAATTTCTTTGTCCCCATGGGCTGTCATCCAGGGCTCATCTTTGCTTTTTGGTCACGCATGTCTT
TATCTCCTTAGTTGTTCTGGAAGATTCCATGTTGCTACAGTGGGTGAAACTCTTGCGTCATCTTGTGGGTATTTTAAAGAAGTTGTTAAATAGC
AAATGGGGATTGTTGACGCTGTGCACGTTTATTTTGATGTTCCAGATAAAAGTAAAGGAAGTTACCGGCAGCATTTTTTCAAGCACGGTGGCAC
AGCGGAACTGAAATGCTCCCAAAAATCCAACCTGGCCCGGGTCTTTTGGAAGTTCCAGAATGGCGTGTTGAAGGCCGAGAGCCCCAAGTACGGT
CTTATGGGCAGAAAAAACTTGCTCATCTTCAACTTGTCAGAAGGAGACAGTGGGGTGTACCAGTGCCTGTCAGAGGAGAGGGTTAAGAACAAAA
CGGTCTTCCAAGTGGTCGCCAAGCACGTCCTGGAAGTGAAGGTGGTTCCAAAGCCCGTAGTGGCCCCCACCTTGTCAGTTGTTCAGACAGAAGG
TAGTAGGATTGCCACCAAAGTGTTGGTGCACATCCACCCAAGGGTCTTCTCCCCCACCCCAGCCGTGCAGGCCACCTCCTCCGGGGCCATCACC
CTTCCTCCCAAGCCTGCGCCCACCGGCACATCCTGCGAACCAAAGATCGTCATCAACACGGTCCCCCAGCTCCACTCGGAGAAAACCATGTATC
TTAAGTCCAGCGACAACCGCCTCCTCATGTCCCTCTTCCTCTTCTTCTTTGTTCTCTTCCTCTGCCTCTTTTTCTACAACTGCTATAAGGGATA
CCTGCCCAGACAGTGCTTGAAATTCCGCTCGGCCCTACTAATTGGGAAGAAGAAGCCCAAGTCAGATTTCTGTGACCGTGAGCAGAGCCTGAAG
GAGACGTTAGTAGAGCCAGGGAGCTTCTCCCAGCAGAATGGGGAGCACCCCAAGCCAGCCCTGGACACCGGCTATGAGACCGAGCAAGACACCA
TCACCAGCAAAGNNNNNNNNNNNNNNNNNNNNNNNNNNNNNNNNNNNNNNNGACAAGCCCTTTGACGTCAAGTGTGAGCTGAAGTTCGCTGACT
CAGACGCAGATCGAGACTGAGGCCGGCTGTGCATCCCCGCTGGTGCCTCGGCTGCGACGTGTCCAGGCCGTGGAGAGTTTTGTGTTTCTCCTGT
TCAGTATCCGAGTCTCGTGCAGTGCTGCGTAGGTTAGCCCGCATCGTGCAGACAACCTCAGTCCTCTTGTCTATTTTCTCTTGGGTTGAGCCTG
TGACTTGGTTTCTCTTTGTCCTTTTGGAAAAATGACAAGCATTGCATCCCAGTCTTGTGTTCCGAAGTCAGTCGGAGTACTTGAAGAAGGCCCA
CCGGCGGCACGCAGTTCCTGAGCCCTTTCTGTAGTGGGGCAAAGGTGGCTGCACCTCTGTTGGCTGAGAAGAGCATCCCTTCAGCTTCCCCTCC
CCGTAGCAGCCACTAAAAGATTATTTAATTCCAGATTGGAAATGACATTTTAGTTTATCAGATTGGTAACTTATCGCCTGTTGTCCAGATTGGC
ACGAACCTTTTCTTCCACTTAATTATTTTTTTAGGATTTTGCTTTGATTGTGTTTATGTCATGGGTCATTTTTTTTTAGTTACAGAAGCAGTTG
TGTTAATATTTAGAAGAAGATGTATATCTTCCAGATTTTGTTATATATTTGGCATAAAATACGGCTTACGTTGCTTAAGATTCTCAGGGATAAA
CTTCCTTTTGCTAAATGCATTCTTTCTGCTTTTAGAAATGTAGACATAAACACTCCCCGGAGCCCACTCACCTTTTTTCTTTTTCTTTTTTTTT
TTTTAACTTTATTCCTTGAGGGAAGCATTGTTTTTGGAGAGATTTTCTTTCTGTACTTCGTTTTACTTTTCTTTTTTTTTAACTTTTACTCTCT
CGAAGAAGAGGACCTTCCCACATCCACGAGGTGGGTTTTGAGCAAGGGAAGGTAGCCTGGATGAGCTGAGTGGAGCCAGGCTGGCCCAGAGCTG
AGATGGGAGTGCGGTACAATCTGGAGCCCACAGCTGTCGGTCAGAACCTCCTGTGAGACAGATGGAACCTTCACAAGGGCGCCTTTGGTTCTCT
GAACATCTCCTTTCTCTTCTTGCTTCAATTGCATACCCACTGCCTGCCCAGACTTTCTATCCAGCCTCACTGAGCTGCCCACTACTGGAAGGGA
ACTGGGCCTCGGTGGCCGGGGCCGCGAGCTGTGACCACAGCACCCTCAAGCATACGGCGCTGTTCCTGCCACTGTCCTGAAGATGTGAATGGGT
GGTACGATTTCAACACTGGTTAATTTCACACTCCATCTCCCCGCTTTGTAAATACCCATCGGGAAGAGACTTTTTTTCCATGGTGAAGAGCAAT
AAACTCTGGATGTTTGTGCGCGTGTGTGGACAGTCTTATCTTCCAGCATGATAGGATTTGACCATTTTGGTGTAAACATTTGTGTTTTATAAGA
TTTACCTTGTTTTTATTTTTCTACTTTGAATTGTATACATTTGGAAAGTACCCAAATAAATGAGAAGCTTCTATCCTTGATTTGGGGAAGACAT
TCCTTAGCGGAGGCTGTGCAGCCTCCCGCCCCTGCATGGCCCAGGCGGGACAGCTGGGCTCGCTCTTTTCTGTCCGTTTCCTGCTGTGTTTTGC
CCTCTCTGGCCTGTGACGTGCTCCTGGCGACTTCTTCCTCAGTGCCCTCTCTTTTCTGTCCTCTCTGTCTCACAGCCTCGTCTCCTAAGCCCCT
CCCTCCTCCTGGCTCCTCTTCCCTGTCCTGTCTGGGCCATGTGGGGGACAGGAGGCTTTCCTCTCCCTGGACCCCCTGGCCAGCCTCGGGTGCG
GGGCCCGACAGCAGCTCGAGGGTCTCCTTGCTGCCGCCCTTCCTGAGTGACCAGGCACAGCACGTGCACGCCCTGGGGAACTTCTACCTCTTCT
GCCAGGCCACAGGTGAGTTGGGCCCTCTCTGTCCCCTGTGCCCTGTCCATCTCTCTCCTCCCACACCTGGGGGGCGGCCGGGGGCATCCTCCAC
ATCCCTCCACATGTTCCTTTACCTTCCTTATCTGTCCTGGCTGCGTCCCTCGGGTTGAGCCGTTTCACGCTTTCATTGTGGGTCCATCCCGAGG
GAAACTGTAGACAGTGTGAAGCCACTTCTAGTGTACAGGGGTCAACCAGTGTTTTCTGTAAAGGGCTAGATATTTAGGCCTTGTGGGCTGCAGG
CTGTGTCACAGCCACTAAGCTCTGCTGTTGTATTGTAAAACCAGCCATAGTCAATACACAACGAGTGGTCAGGGCTGTGTGCCAGTAAAACTTT
ATTTATAAAAATGCACGGTGGGCCAGATTTGCCCCAAAGGCCCCAGTTTCCCAACCTGTGGTATATAGGATTGCTTCTCCCTTAAAAAAAAAAA
AAATTTCAAGAGACAGGGTCTCCCTCTGTCACCAAGGCTGGAGTGCAGTGACACAATCATAGCTCACTGCAGCCCCTGCCTCCTGGGCTTAAGC
CATCCTCCTGCCTCAGCCTCCAGAGCCAGAGTGCTTCTATTTCTGCTAACAATTTGGAGGTTGATATACGTGGTATTTGGAAATTTTAAAAAAT
TAAGAGCTTCCAGAATGGGAGTTGTTTTAATGACTTGCTTCGTGGTATTTAAAAGCGGAAAAGATTGGCGGGCATAATCCCCCTGCTTTGGGAG
GCCAAGGCAGGAGGATTGCTTGAAGCCAGGAGTTTCACAGCAGCTTGAGCAACATAGCGACACCTGATCTCTACCCAAAAATGTGAAAAAAACT
AACCAGGCTTGGTGGCACGTGCCTTGAGGATTGTTTGAGCCCAGGAGTTTGAGGTTACAGTAAGGTATGATGGTGCCACTGCACTCCAGCCTAG
ATCACAGAACAAGACCCTGTCTCAAAAATCATCATCATTATAAAAACAGAAAAGATTGAGAGACTAGTGTACTCCTCTGGGACCTGGAGGCATT
TGCCCTGGATTGGAAAATACTCGGATGGGATATCTGCTCTTGAAAGATAGTAGATGTCCAGAACCACACTTCAAAGATGTATCCTGTGCTTCAG
GCAGTGCGTGGTAGCCCTGGTGGCCCCTGTGTTGTCATCTGACTGATCAGTAGATACAGAAAGAAAGGAAGTGGCCTGTTAGAGCCTGCCCCGG
GGGAGGGATGAAGTTAATGAAGAGGAAAAGCAAACCCAATTGCTCTCTTTTTAAACATGTTAAACACACTGTCAGTAGCTCCTGAGAGTTTTAA
GGTGCTGCCCTCTCCTGTTAAACAAGCTCTGAGAGCCCCAGGTGGCCCATGTCTGCTGAGGCCAGAATGTCCCTTGGCAGCCAGNNNNNNNNNN
NNNNNNNNNNNNNNNNNNNNNNNNNNNNNNNNNNNNNNNNNNNNNNNNNNNNNNNNNNNNNNNNNNNNNNNNNNNCAGCCAGCACTGGACAGGA
GAAGCTGCCGGTGAGCATGGAGCACTGTGGAGACGGAGCTTTGCTAGGCTGGCTTGTGTGGTTCCTGCCCTCCACTGGGTGCTACGGACCAAGG
GCTGTGCTGAGCCCCCTGTGGCCGCTCTCACAGCTGAATGGTGCTCCTCGGCCAGCTAGTTTGCCTTGGTGCAGGCATCCCAGAGGACTTCATG
TCTCCATGGAGACTGTGTCCTGTCGCAGCACTGGGGTCCTTCAGGCAGACTCAGGAAGCTCCTCCTCGGTGGGGAGGCTGGGAAGCGGGCGGGT
CTCTATGAACTGGCCGTGGACCCAGGGCTCCTTTGCGGGCAGATGCAGGCAGGCTGTATGGTCAGGCAATGTTGGTTGGTTATTTATTTATTTA
TTTTTGAGGCAGAGTCTTGCTCTGTTGCCCAGGCTGGAGTGCAGTGGCACGATCTTGGCTCACTGCAAGCTTCGCCTCCCAGGATCAAGCGATC
CTCCTGCATCAGCCCCCCTAGTAGCTGGGATTACAGGCACACCTCACCATGCCCAGCTAATTTTTGTATTTTTAGTAGAGACAGGGTTTCGCCA
TCTTGGCCAGGCTGGTCTCAAACTACTGACCTCAGGGGATCCACCTGCCTTGGCCTCCCAAAGTGCTGGGATTACAGGCATGAGCCACCATGCC
CGGCTGCTGTTACATTTTTAAATGTGAGGCTCCAGGTGTACCCCCTGTAGCTAGGCAGGCATACACACAAGCCCCTGGGGTCAGCAGGGAATGA
AAGATGGGTGCCCAGGGAGAAGGGACATCACTGTTACAGCAACAGAAACCACCCCAGATATTTCAACCACAGGGCATTTAACACAGGGAATTGG
TTACAAAGGTGATAGAAGGGCTGGGAACAGTGGGAAGGTGAGGTTTCCATTATCAGCCATGGGCAGGAAGAGTGAAGTGTCAAACACTCAGTCT
CACTTGCTGTGCCACCGAGGCTGTGGCCGTGTGATCTGCAGCCTCTCCCTGCCCGCCACACTGCCTTCACTGCTGGAGGTCCTACTTGAAGCCA
CAAAAATGTTTCTTCCTTCCAACTTCCTGTCTCCTGTCAATGACTCCCATGGCCAGACCTAACAGAAAGCTGGCGGTCACAGATGGATATGGGG
AATGTAGCTGTGGGCTTGCAGCAGTGTGGCCAGGAGTAGTGTGTAGAGGGTAGGGGAGCTGAGGGAGGAGGGCAGTCCTTTGCCAGTGCTCAGG
ATGGACTGAACAGATTGAGAACTGAGGCCCCCAGCCCTCCGCCACCCCCTTTTCACGTGGTCTTTACTTTCTCTCCAGAGGCATTTGGCCCCAT
CTGGGACATTCTCGTTCACTTGTTCCACTGAGGCCACAGCCTGCGAAGGCTGTGAGACCTCCCCAGGGAGCACCAGCTTAGGAGAGAGTGGCTT
CAGCCTTTTCAAGGGTGGGCCGAGGCCAGGGCATCATGGGCATGCCGTGAACTCAGAGGGGCTGTGGCTGTGAATTTCCCGAGTAGGCTCCAGG
GTTCTGTTGGGGTCTGTAGATGCTTCCTGCGACAGAGGCCACAGTGTGACTTGCAAGTCCCAGAGACTTTCCTGAGGAGAGTGAGTGCCATTTT
CTGCCTTGTGGGCCAGGAATGAGCCATTCCAGGTGAGGCCCAGAGCCTGGAGATTGCAATACAGTGCACTCAAGGCACAGGCCTACCGCTTCTG
CCTGTGTCCTCCCCCAGCCACCCAGGGCAGGGCTGCTTTGGAACTTGGCTGGGTTTTTCTTTGCTATAATGGCTGTTTTCGTGGTAGCTGTCAC
AGTGGTTTGGGGGCTGTCTGATTAAAGGGCTAGCAAAGTGTCTTTAGAATATGAAACTTAAGAGGACATTCGTGGCATCTTCAGTTCCCCATGC
TGCGTCCTGGTGCCTGTAGCTCTGCTGGGTGACCCTGTGTCTTGGGGAGCGTCTCACCCCCCAGAGAGGGCTGACTGATGGAAGGTCAGCTGGT
CAGGGTTGCACTTGGGGACTGGGTCTGCCCCAGAGCTGGTGGAGGCCCATGTGTCACCCAGGAGATGTCCCCACCTGCCCCAACACTTGGCCAG
TGCTCAGAAAGTGGCCCCAGGGACTTGGCCATCCAGGAGAGGTGAGGGCCCTCTGAGGCCAAAGCGCTTCCTGAAAGGGGATCGGGGTGGGTCT
GCACAGGGGAAAACATAGCCTGTGGTGTGTGCAGAGGCTGCCAGGGCACAAGGGCCCAACGTGGGGTGCGGTGGAGGTGATGTGGCCACGTGGG
CGGAGTAGGGCGGAGAGGCCTGTGTCCTGGGGATGCGAGAGCTTGAGGCCTGGTGGTGGGGTGGAGGCCCACCCTTGTCCTGCACCCAGGGGCC
TCAGTGCCCATCCAAGGGAGGGACAGGGCCACACCTGTGGGCCTGTGTCAGGAGCTGGGGTGGGAGATTGGCCACCAGTGCCACTCTCCACCTA
GACACATTTCATCCCTTCTCCACCACAGTTCTCGCCTCTAGGAAGTGGAATTATCAGTGGAAGATGATACGGAAAGATCCACTGATGCCCCTGG
CACAGGCCAGCCTTGTCCCTGGGGCAGGATGGGACGGTCCTGAGAAGGCCCTGGGACACCCTAGGGGTGTCATAGTGGGAACTATGCCTTTGCA
GTGCCCAGGTGAGACAGCACATGGTGTCACCCCAAGCATGGTACCCTGGGCTCAGTGACAAGAGGGGTGGGCAGCTCACGACTGCACTGCCTTG
GGGCCCGTGCCCTGGGAGCCGCTAGCCCTATGATGCCTGAGTACCTGGGGGACTGAGTGCATCGGCAGGGCCTGGAGCAGTGGGAGTGGCCGAN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNACCAAACACAACCACTCACACATGCACC
ACACACAGCACAGCATACAACACACATCACACACACACCAATCATACCACTGTTACACACACACCACCACACATACTGCACACACCAAAGCACA
TACACACCACATAGACACATGCACGCTACCCACACCACACCAATCACATCCACACATACACAACCATACACACACACACACACATTGTTGACTT
AGGATCGTTTCAACTCACAGTCGGTTTATTGGGATGCAGCCCCGTGGCAAGTGGAGCAGCATGTGTATTGACAAGGGCAGGAAAGCCCTCTGGA
GCTGGTGAAGTAAAGTTAGCATGCAGCCGAGCTCTGCTCGTCTGAACTGATGAACCCAAGTTCCTCATGGATCACTCAGAATACAGATGGGTGA
AAAAATAACCCCACCACCCGTGTGCCAGCACATAGCGCATCTGGCATACAAACGTAAGGATCAATAGCGATGAATGGCGATGGGTTGTGTCTAC
ATGCAGATACAAATCATGCACTTGTACATTAGTGTGCACACACAGAAACAGATCGATATAATTTTTTAACCAAAGTGCAGTCTTTACCCTGTCA
GGGCCCTGCCTTCACCTCTGCCAGGACTATCTTCCATGTCAAGTTTAGACCCGTGTCATTTTCAAAGCTAATAATGGTTCCCCCATACTTTAGG
CAGCATTTAGATATTTCCATTTGGGATGGCCTTTTATGTTTTAACTACAGTGTTAGAAATCAGAGCCCTTCCAACCACTGCGATCAGAGGGCAG
CAGCAGAAGGCGCCCTTGGTCCTCTTTGAGGGGCCCAGGGCTCCGGAGAGGGCCCCCGTTCCTAGCACCCACAGTGGCCTGTTGGCACGCATGT
GTGTGTTCCAGCTGCTCTGTGACATTTCCCTTTGTTTCAGACTCTTGAAAGGCCACGGCCGTCGTGGGAATTAGTTGACCTTGTGTGTTGGGCC
ACCCTTGCTGGACAGGACAAGGTCGTCCTATCAACTCCCTGTGCTCTGAGGTCCTTCGCATGTTAGATGACGCAGCTTCACCACAAACTATCCA
TGTCTCTTCTGTTATCTGGGATAATTCCACGTGCCTTTACAACACAGCTGTCTGGTTACCTGGGGTCTGGAAATGCCCTCGGTGCCTCAGTGGT
GCCTTCCCACAGGCCCCCCATGGGAGCCAGGCGATGCCTACAGATGCCTCAGTGGTACTTTCTCAGGTCCCTTTGCAGAGAAGACGTTCACGAT
TCCTGCCCACATTGATAAGGCGGCTTGGCCAGTAAGTGGCAGGCATGAACAGGTGCATTCCAGAAGCGCTGGGGCTGGTCCAGTGCCCTGAGTG
GTCTAGAGCATACCAGAGTGAAACGTTAGGCTGCGTCGGCCTGGTGACTGTCAGGGAGATGAGGACTCAGAGAAGGGAGCTGTCAGAATTGCTA
CCTATGTGGCAGGGGTCTCACTCTCTCCTCCCTCCTCTTGGGGCCCCTGCCTACTCTGTGACTTCAACAGAGGACAGAAAGGATGACCAACAAG
GCCTCAGGGCATACCCCCAGCTGCACTGTCCTTGTCCCCAGAAGGGTCCCCAGAGTCTGGGAGGGAGGAGGGGCCATGGCCAGGATTAGAAAAG
ACCGTGCCCCAAGGAGAGAGCACAGAGATGGGCCAGGGAAGCAGTGGGATCCTCGGGAAAAGAGGCTGGGAGCAGGTGTCCGGGTGAGGCCCGG
GCAGCGGGTACAGGCAGGGACAAGCCAGGAGTCCCATCATGGAAGACATCATGGCCAGCGGCCAGCACTGTGTGCAGAGAATGGCCCCTCTGGG
GATGTGCCCGCTGCCCCATTAGTTGCAGGAAGGCCACCTGGATGTGAGTTTGGGACATGGGCTGGCCCCGAGGCAGCCAGCCCTGAGGTCACAG
TGCGCCACATGCATGGGGATGCCGGGAGGACACAGAGGAGTGCTGCAGGAGATGGCAGTCGCGGCACTTTCCAGGATGGGCCCGAGCAACTCAG
TCACTGGGTGGATGTCTCACCACCCCTCCAGGCCTTCCTCAGGGCGAGGCCACTGCCCAGAGGGCACCTGGTTTGTCGCAGAGTAGAATCAGCT
CCATATTTTGTAATCACATCCACGAAGCTCTGAAGCCATGGTGGTCCTGGCACCAGCTGGCTCCGTTAGAGGCTTGGGGTGGGAGTGAGCAGGT
GGGAGGTGCCCCAGTGACAGGCTGAGTGCTGGAGCCCAGGCTGCCAACAGCCTCCCCAGACTCCTGAGTTGATGTAATAAACCCAGCCCTGGTG
AATAAACCTGGCCCTTGCTTATACCAAGGTGATCAGTCTTCTCACGACAGTCCTAGCTGTAGGAGAAAGATCATTACCTACGGCATCTGCCACA
GCAGTTAATTTGCCTTCAACTTAACCTAGTTCCACTGACAGGAAGGGCCAGGGGGAAATATCTGTCCTCAGGACCTCTACCCTAGATCCTTGTA
AGCCACAACAACTTCCCTTCTTTGAGGGCGCGACCAGTGTCACCTGGCTCGCCACTGGGAAGAGCCAAGGCACATCCAGGTTGGTGGCTGCTGA
GCCCCACAGCATCAGACCAGCCTCTTGTCCCCACCCCACAGAGGCCAAGGGGGTGATCTGGGAGTAGCACCCCAGCCTGGCCGCTCTCTTGAGG
TAACAGGGTTGGATGGTTAGGGAGCTTGGAGCACTGGCCCAAGCAAGAAACCAGTGGTGTTTTCAGGAGGCCCTGCTGGGAGATGGAAGACAGC
TCCCCACTGGGGGCCTAGGATTCCAGGAGCTCCCCAGCAGTCCTCCGTGGGACATACGTGGGTTTGCACCTGCTGCCCTGTCCCGCTGAGGGTG
CGGTGCTGATGGGGTGGCCCTGCACACTGATGGTAACTCTCACTTTCCCTCTCTCTCATGCATTCAGAAAGAGATGGATAGTTTAAAAACGAAT
ACATGGAACGTAAGTACTTCTTTAACATCTTTAATGTCTTATGATGATCAAGCATGTATTTAAATAGTATTTACTCATCCCTGTTAGCGGTGGT
AAGCACGTGTCTGTTTCTGCTCACACCCCAAGAGTGTAGAGCTTTGAGGGTTTTGCAGTCAGTGGTCCCATGATGGGGCGTTGGGTGATTAATC
CAATCCAGCTTAGGCCACCCAGATTCTTAGTCTCAAGGGCTTGGTCATGAGAACCTTCCACAGGCTCCTGTCACTCAGTGACTCCAGTCCCCTG
GGTGGTTCTTGGTGCAGGTCACATGAGGGAGGAGACCGGGCTTTTATAGAACTCACTTCACTGTCTGTTCAAAGAAAAGTGTTACTTGATCATA
ACAGCTCAAAAAAATCTGTTTCTTAAAATGAGATTTTACCTGGATGCCCATCAGCTGGGAAAGGGAATATCTATTCCCCAGTTGATGGGCACCT
AGATGTACATAGAGACATATGTTTGTACATCTATGTAATATGTACTTATATTTGTAGATAGATACATGGAATTATCTGTGGCCAGAGTCCAAAA
CATTTTGAGTGGGAAAAAACAAATTATAGAGTGTTAAGTTTTGTTTTTTTTTAAATTCTGCACAAAAACCAAACCTGTTCACAAGGCCTTGTCC
ACCAGGCCAGGGCCAGATGGGCCGCCAGGCAAAACAGTTTTGGAGCACCTTCTGAATTTCCTTGAGTTTACAGAATGTTTCCTTCAAAGTAACA
CTTTTATAAAAAATTATTATTATAAAAGCAATGCTTGTTTGTTATAAGTTTGGAAACTCCAGGGAAGTATAAAGAAAAAATTTAAACAATCCCA
AATTTAACCATCCAGACGTAGTTACCACCAGCATCCCGGCCATTTTTGGGCCATGACTATCTACGTTCACACATGTATACATGGGTGTGTACAT
ATATGCACACATTCCTAGATATTGTTTCACTTATTTTTCTGTGTTGCTGAATATTCTTCGATGGCACTTCGAATGAGTGACTTGTGCCCCCCTC
CTTGGACATTTCCCAACTCCTCACTGATAGAAACAGGGCTTCACTGGGCCCATCCTTGCCTCGGGACTCATTCCTAGCACTGCCCAGCTAAAGG
GTCTGAGCTTGGTCAAGTCCTTGGGAGGAGCATTCTCTGGTGATCCTGGACAGTGGGGAATTGTTGACCAAGAGACCGGACAAAGATGAGCAGA
TGCCACAGTCAGTAAAGCAGGGAAAATGCATTACTAAAGTCAGCTTCATTAAACTGGCGATCGCTTCTTTTCAAAAGTCCAGCATGTGCCACAG
AAATGGAAAGCTCTCTCCCCACTACTGTCCTGCCCAGGGAGGGGAGCGTGGGTCCACCCACACCATTTAATACCTGTAACTGGATGCACCTCTG
CCCCTACACCTGAGAACAAGGCCAGTTTTTGTGTGTGCAGGTACTTTGGACTCTTTTGGCTCTGGAATTTGTTTTACCTGCTCAGTGTTCTGAA
CTCCTTCCTTTTCACTACTAAGCATTCTCCATGAAGCCACACTTCATTTTGTCATTGTGAGGGTGATCGGCATCTAGGGCAAATCTAANNNNNN
NNNNNNNNNNNNNNNNNNNNNNNNNNNNNNNNNNNNNNNNNNNNNNNNNNNNNNNNNNNNNNNACCAATTCTTACGAGGTGTCATGGTCAAGCA
GAGGCCATGGCCGGCACTGGTCTAGGTGGCCCATGGCCCATTTGGGGGCCTTGGTGGGCCTCGAGGAAGGGCAGGTGCTGGTCAGGGAGCCGGG
TCGCTAGGCAGCAGGTGGGTGTCACCCCAGTCCACGCTGCTGCGGTGCCTGGCCTGATGCAGGCTGTGCCTGAGGGAGACAGAAGCAGCAGAGT
AGGCTGGCCGGCCTCCCCAGCCCCACCACAAGCAGGAGCTTCAGAGACAGAAGGAGCACCTGCACCCCCTCACCTGCCCTCCAGGGCCTGGCCT
GGCCCTCCCAGCTCAACTCCAG
HUMAN SEQUENCE—mRNA
CTGAGCCGCATCTGCAATAGCACACTTGCCCGGCCACCTGCTGCCGTGAGCCTTTGCTGCTGAAGCCCCTGGGGTCGCCTCTACCTGATGAGGA
TGTGCACCCCCATTAGGGGGCTGCTCATGCCCCTTGCAGTGATGTTTGGGACACCGATGGCATTTGCACCCATACCCCGGATCACCTGGGACCA
CAGAGAGGTGCACCTCGTGCAGTTTCATGAGCCAGACATCTACAACTACTCAGCCTTGCTTCTGAGCGAGGACAAGGACACCTTGTACATAGGT
GCCCGGGAGGCGGTCTTCGCTGTGAACGCACTCAACATCTCCGAGAAGCAGCATGAGGTGTATTGGAAGGTCTCAGAAGACAAAAAAGCAAAAT
GTGCAGAAAAGGGGAAATCAAAACAGACAGAGTGCCTCAACTACATCCGGGTCCTGCAGCCACTCAGCGCCACTTCCCTTTACGTGTCTGGGAC
CAACGCATTCCAGCCGGCCTGTGACCACCTGAACTTAACATCCTTTAAGTTTCTGGGGAAAAATGAAGATGGCAAAGGAAGATGTCCCTTTGAC
CCAGCACACAGCTACACATCCGTCATGGTTGATGGAGAACTTTATTCGGGGACGTCGTATAATTTTTTGGGAAGTGAACCCATCATCTCCCGAA
ATTCTTCCCACAGTCCTCTGAGGACAGAATATGCAATCCCTTGCCTCAACGAGCCTAGTTTCGTGTTTGCTGACGTGATCCGAAAAAGCCCAGA
CAGCCCCGACGGCGAGGATGACAGGGTCTACTTCTTCTTCACGGAGGTGTCTCTGCAGTATGAGTTTGTGTTCAGGGTGCTGATCCCACGGATA
GCAAGAGTGTGCAAGGGGGACCAGCGCGGCCTGAGGACCTTGCAGAAGAAATCGACCTCCTTCCTGAAAGCCCGACTCATCTCCTCCCGGCCAG
ACAGCGGCTTGGTCTTCAATGTGCTGCGGGATGTCTTCGTGCTCAGGTCCCCGGGCCTGAAGGTGCCTGTGTTCTATGCACTCTTCACCCCACA
GCTGAACAACGTGGGGCTGTCGGCAGTGTGCGCCTACAACCTGTCCACAGCCGAGGAGGTCTTCTCCCACGGGAAGTACATGCAGAGCACCACA
GTGGAGCAGTCCCACACCAAGTGGGTGCGCTATAATGGCCCGGTACCCAAGCCGCGGCCTGGAGCGTGCATCGACAGCGAGGCACGGGCCGCCA
ACTACACCAGCTCCTTGAATTTGCCAGACAAGACGCTGCAGTTCGTTAAAGACCACCCTTTGATGGATGACTCGGTAACCCCAATAGACAACAG
GCCCAGGTTAATCAAGAAAGATGTGAACTACACCCAGATCGTGGTGGACCGGACCCAGGCCCTGGATGCGACTGTCTATGATGTCATGTTTGTC
AGCACAGACCGGGGAGCTCTGCACAAAGCCATCAGCCTCGAGCACGCTGTTCACATCATCGAGGAGACCCAGCTCTTCCAGGACTTTGAGCCAG
TCCAGACCCTGCTGCTGTCTTCAAAGAAGGGCAACAGGTTTGTCTATGCTGGCTCTAACTCGGGCGTGGTCCAGGCCCCGCTGGCCTTCTGTGG
GAAGCACGGCACCTGCGAGGACTGTGTGCTGGCGCGGGACCCCTACTGCGCCTGCAGCCCGCCCACAGCGACCTGCGTGGCTCTGCACCAGACC
GAGAGCCCCAGCAGGGGTTTGATTCAGGAGATGAGCGGCGATGCTTCTGTGTGCCCGGATAAAAGTAAAGGAAGTTACCGGCAGCATTTTTTCA
AGCACGGTGGCACAGCGGAACTGAAATGCTCCCAAAAATCCAACCTGGCCCGGGTCTTTTCGAAGTTCCAGAATGGCGTGTTGAAGGCCGAGAG
CCCCAAGTACGGTCTTATGGGCAGAAAAAACTTGCTCATCTTCAACTTGTCAGAAGGAGACAGTGGGGTGTACCAGTGCCTGTCAGAGGAGAGG
GTTAAGAACAAAACGGTCTTCCAAGTGGTCGCCAAGCACGTCCTGGAAGTGAAGGTCGTTCCAAAGCCCGTAGTGGCCCCCACCTTGTCAGTTG
TTCAGACAGAAGGTAGTAGGATTGCCACCAAAGTGTTGGTGGCATCCACCCAAGGGTCTTCTCCCCCAACCCCAGCCGTGCAGGCCACCTCCTC
CGGGGCCATCACCCTTCCTCCCAAGCCTGCGCCCACCGGCACATCCTGCGAACCAAAGATCGTCATCAACACGGTCCCCCAGCTCCACTCGGAG
AAAACCATGTATCTTAAGTCCAGCGACAACCGCCTCCTCATGTCCCTCTTCCTCTTCTTCTTTGTTCTCTTCCTCTGCCTCTTTTTCTACAACT
GCTATAAGGGATACCTCCCCAGACAGTGCTTGAAATTCCGCTCGCCCCTACTAATTGGGAAGAAGAAGCCCAAGTCAGATTTCTGTCACCGTGA
GCAGAGCCTGAAGCAGACGTTAGTAGAGCCAGGGAGCTTCTCCCAGCAGAATGGGGAGCACCCCAAGCCAGCCCTGGACACCGGCTATGAGACC
GAGCAAGACACCATCACCAGCAAAGTCCCCACGCATAGCGAGGACTCACAGAGGATCGACGACCTTTCTGCCAGGGACAAGCCCTTTGACGTCA
AGTGTGAGCTGAAGTTCGCTGACTCAGACGCAGATGGAGACTGAGGCCGGCTGTGCATCCCCGCTGGTGCCTCGGCTGCGACGTGTCCAGGCGT
GGAGAGTTTTGTGTTTCTCCTGTTCAGTATCCGAGTCTCGTGCAGTGCTGCGTAGGTTAGCCCGCATCGTGCAGACAACCTCAGTCCTCTTGTC
TATTTTCTCTTGGGTTGAGCCTGTGACTTGGTTTCTCTTTGTCCTTTTGGAAAAATGACAAGCATTGCATCCCAGTCTTGTGTTCCGAAGTCAG
TCGGAGTACTTGAAGAAGGCCCACGGGCGGCACGGAGTTCCTGAGCCCTTTCTGTAGTGGGGGAAAGGTGGCTGGACCTCTGTTGGCTGAGAAG
AGCATCCCTTCAGCTTCCCCTCCCCGTAGCAGCCACTAAAAGATTATTTAATTCCAGATTGGAAATGACATTTTAGTTTATCAGATTGGTAACT
TATCGCCTGTTGTCCAGATTGGCACGAACCTTTTCTTCCACTTAATTATTTTTTTAGGATTTTGCTTTGATTGTGTTTATGTCATGGGTCATTT
TTTTTTAGTTACAGAAGCAGTTGTGTTAATATTTAGAAGAAGATGTATATCTTCCAGATTTTGTTATATATTTGGCATAAAATACGGCTTACGT
TGCTTAAGATTCTCAGGGATAAACTTCCTTTTGCTAAATGCATTCTTTCTGCTTTTAGAAATGTAGACATAAACACTCCCCGGAGCCCACTCAC
CTTTTTTCTTTTTCTTTTTTTTTTTTTAACTTTATTCCTTGAGGGAAGCATTGTTTTTGGAGAGATTTTCTTTCTGTACTTCGTTTTACTTTTC
TTTTTTTTTAACTTTTACTCTCTCGAAGAAGAGGACCTTCCCACATCCACGAGGTGGGTTTTGAGCAAGGGAAGGTAGCCTGGATGAGCTGAGT
GGAGCCAGGCTGGCCCAGAGCTGAGATGGGAGTGCGGTACAATCTGGAGCCCACAGCTGTCGGTCAGAACCTCCTGTGAGACAGATGGAACCTT
CACAAGGGCGCCTTTGGTTCTCTGAACATCTCCTTTCTCTTCTTGCTTCAATTGCTTACCCACTGCCTGCCCAGACTTTCTATCCAGCCTCACT
GAGCTGCCCACTACTGGAAGGGAACTGGGCCTCGGTGGCCGGGGCCGCGAGCTGTGACCACAGCACCCTCAAGCATACGGCGCTGTTCCTGCCA
CTGTCCTGAAGATGTGAATGGGTGGTACGATTTCAACACTGGTTAATTTCACACTCCATCTCCCCGCTTTGTAAATACCCATCGGGAAGAGACT
TTTTTTCCATGGTGAAGAGCAATAAACTCTGGATGTTTGTGCGCGTGTGTGGACAGTCTTATCTTCCAGCATGATAGGATTTGACCATTTTGGT
GTAAACATTTGTGTTTTATAAGATTTACCTTGTTTTTATTTTTCTACTTTGAATTGTATACATTTGGAAAGTACCCAAATAAATGAGAAGCTTC
TATCCTTAAAAAAAAAAAAAA
HUMAN SEQUENCE—CODING
ATGAGGATGTGCACCCCCATTAGGGGGCTGCTCATGGCCCTTGCAGTGATGTTTGGGACAGCGATGGCATTTGCACCCATACCCCGGATCACCT
GGGAGCACAGAGAGGTGCACCTGGTGCAGTTTCATGACCCAGACATCTACAACTACTCAGCCTTGCTGCTGAGCGAGGACAAGGACACCTTGTA
CATAGGTGCCCGGGAGGCGGTCTTCGCTGTGAACGCACTCAACATCTCCGAGAAGCAGCATGAGGTGTATTGGAAGGTCTCAGAAGACAAAAAA
GCAAAATGTGCAGAAAAGGGGAAATCAAAACAGACAGAGTGCCTCAACTACATCCGGGTGCTGCAGCCACTCAGCGCCACTTCCCTTTACGTGT
GTGGGACCAACGCATTCCAGCCGGCCTGTGACCACCTCAACTTAACATCCTTTAAGTTTCTGGGGAAAAATGAAGATGGCAAAGGAAGATGTCC
CTTTGACCCAGCACACAGCTACACATCCGTCATGGTTGATGGAGAACTTTATTCGGGGACGTCGTATAATTTTTTGGGAAGTGAACCCATCATC
TCCCGAAATTCTTCCCACAGTCCTCTGAGGACAGAATATGCAATCCCTTGGCTGAACGAGCCTAGTTTCGTGTTTGCTGACGTGATCCGAAAAA
GCCCAGACAGCCCCGACGGCGAGGATGACAGGGTCTACTTCTTCTTCACGGAGGTGTCTGTGGAGTATGAGTTTGTGTTCAGGGTGCTGATCCC
ACGGATAGCAAGAGTGTGCAAGGGGGACCAGGGCGGCCTGAGGACCTTGCAGAAGAAATGGACCTCCTTCCTGAAAGCCCGACTCATCTGCTCC
CGGCCAGACAGCGGCTTGGTCTTCAATGTGCTGCGGGATGTCTTCGTGCTCAGGTCCCCGGGCCTGAAGGTGCCTGTGTTCTATGCACTCTTCA
CCCCACAGCTGAACAACGTGGGGCTGTCGGCAGTGTGCGCCTACAACCTGTCCACAGCCGAGGAGGTCTTCTCCCACGGGAAGTACATGCAGAG
CACCACAGTGGAGCAGTCCCACACCAAGTGGGTGCGCTATAATGGCCCGGTACCCAAGCCGCGGCCTGGAGCGTGCATCGACAGCGAGGCACGG
GCCGCCAACTACACCAGCTCCTTGAATTTGCCAGACAAGACGCTGCAGTTCGTTAAAGACCACCCTTTGATGGATGACTCGGTAACCCCAATAG
ACAACAGGCCCAGGTTAATCAAGAAAGATGTGAACTACACCCAGATCGTGGTGGACCCGACCCAGGCCCTGGATGGGACTGTCTATGATGTCAT
GTTTGTCAGCACAGACCGGGGAGCTCTGCACAAAGCCATCAGCCTCGAGCACGCTGTTCACATCATCGAGGAGACCCAGCTCTTCCAGGACTTT
GAGCCAGTCCAGACCCTGCTGCTGTCTTCAAAGAAGGGCAACAGGTTTGTCTATGCTGGCTCTAACTCGGGCGTGGTCCAGGCCCCGCTGGCCT
TCTGTGGGAACCACGGCACCTGCGAGGACTGTGTGCTGGCGCGGGACCCCTACTGCGCCTGGAGCCCGCCCACAGCGACCTGCGTGGCTCTGCA
CCAGACCGAGAGCCCCAGCAGGGGTTTGATTCAGGAGATGAGCGGCGATGCTTCTGTGTGCCCGGATAAAAGTAAAGGAAGTTACCGGCAGCAT
TTTTTCAAGCACGGTGGCACAGCGGAACTGAAATGCTCCCAAAAATCCAACCTGGCCCGGGTCTTTTGGAAGTTCCAGAATGGCGTGTTGAAGG
CCGAGAGCCCCAAGTACGGTCTTATGGGCAGAAAAAACTTGCTCATCTTCAACTTGTCAGAAGGAGACAGTGGGGTGTACCAGTGCCTGTCAGA
GGAGAGGGTTAAGAACAAAACGGTCTTCCAAGTGGTCGCCAAGCACGTCCTGGAAGTGAAGGTGGTTCCAAAGCCCGTAGTGGCCCCCACCTTG
TCAGTTGTTCAGACAGAAGGTAGTAGGATTGCCACCAAAGTGTTGGTGGCATCCACCCAAGGGTCTTCTCCCCCAACCCCAGCCGTGCAGGCCA
CCTCCTCCGGGGCCATCACCCTTCCTCCCAAGCCTGCGCCCACCGGCACATCCTGCGAACCAAAGATCGTCATCAACACGGTCCCCCAGCTCCA
CTCGGAGAAAACCATGTATCTTAAGTCCAGCGACAACCGCCTCCTCATGTCCCTCTTCCTCTTCTTCTTTGTTCTCTTCCTCTGCCTCTTTTTC
TACAACTGCTATAAGGGATACCTGCCCAGACAGTGCTTGAAATTCCGCTCGGCCCTACTAATTGGGAAGAAGAAGCCCAAGTCAGATTTCTGTG
ACCGTGAGCAGAGCCTGAACGAGACGTTAGTAGAGCCAGGGAGCTTCTCCCAGCAGAATGGGGAGCACCCCAAGCCAGCCCTGGACACCGGCTA
TGAGACCGAGCAAGACACCATCACCAGCAAAGTCCCCACGGATAGGGAGGACTCACAGAGGATCGACGACCTTTCTGCCAGGGACAAGCCCTTT
GACGTCAAGTGTGAGCTGAAGTTCGCTGACTCAGACGCAGATGGAGACTGA

TABLE 29
MOUSE NOMENCLATURE

| | |
|---|---|
| ICSGNM | Mdm2 |
| Celera | mCG3393 |

HUMAN NOMENCLATURE

| | |
|---|---|
| HGNC | MDM2 |
| Celera | hCG19697 |

MOUSE SEQUENCE—GENOMIC
AGCGACGCAGAGCACGTGAATCTCTGAGTTCAAGCACAGCCTGGTCCCGACAGCCAGGGCTTTATAGACAAACCCTGTTACAAAACAAAAACAA
ATATGTATCAATTTAGAGAGGGTCTAACATAACTAGTGTGGTTTTGCTCTCTCGGGATGGACAATTCTCCTCTTGGGTAAAGTGAGAAACTAAA
GGCAGCCTAGAGGAAAATGTTTTGCACTCTGTGTTCCAAGCTAGGATCTGGGAGGAGGACGGCTCCATAGAGAGCGTGTTAGAGAAATGTCTTT
GCGAAGCTGTGTGGGTTTTGTTAGCCAAGCAGTGCCGTCATCCGCCATAACCCAAACACTCCTGTCACCACACTAATGCATCTATTCAGCAAAG
CAAGTCACACTACTAGAAGCCCTCATTGTTGGTCACCAGATATGGTGACACACATGTTTACTCCTAGCATTCAGGTGACAGAGGTAGGTAGATC
TCTCTGACCCTCAGCCTAGCCTGCTCTACATGGCGAACTCCAAGGCAGCCAAGGTTTTTGTAAGTAAGACTCTATCTCAAAATACAAAATAACG
CCCCCACCCCCACCCCAAGAAACTGTTGGTCTTTTATAGAGAATCCTTGCTCACTCAAACAGCTTTCAGCATAAGAATATTCAAAATTAATTGG
CAACCGCTCCCACCATGGGTGTCCCTAATTACACATACCTTGTCATATTTACGAGTGTGATGAACTTTTAGCCCGTGTCACATACACTGATAGC
CATCTCCATAGTGACATGTATTTAGATCACATTACAGTCACGACTCATAGTTTGTTTTTTTAAAATGCTCTTAAAAATTCGACTAAAACAAAAA
TAAAATAAAATAAAACAAAACAAAGTCAACGCAAGCTTTCCTTTCCTAAAGTCTCTGCTGTGCCCGCAGTTTAGACAGAGTTTCTGTCTTCTGA
CTCCGCTCACTCCACACCAGCCAAGTCCTGGTCCCAGTGAACCAATGTTCTGTCTAAGCAGAACACGCTCCAGTAGACAAGCCTTCTTTTCATA
GACTAGCAAACTACATTCCTTTCAGCCTTACGTCTGATGGAGGAAATCATTCACCTCAGGAAACAAGTAAGCCTTTCAAAACAGCACACAGAAC
TTTACTGAACCTTTAATAAAAACATAGGACTAAAAATTACATTCTTTTTTAAAAATTAGGACAAATTTTAATATACATTTATCTTATGTGTGTG
TACGTGTATGTGCGCATATGTATATATGTGTGTGTATGTATATGTCTATATACATTTATCTTGTGTGTGTACGTGTATGTGTGCATATGTATAT
GTGTGTGTGTGTGTGTGTGTATTTCTCTGTGTATATATGTATGTATGTGTGTGCATGTATGGATATGTCTGTATGTGTTTGTGTGTATGTATGT
GTGTAATGTGTGTATGTATGTATATGTCTGTATGTATGTGTGTGTATGTGTGTATGTATGTGTATGTCTATATGTGTTCCTGTGTGTGTATGTG
TCTTTGTGTCTATGTGCATGTGCATGTGTGTGTATGTGCATGCTCAAAGCAATCATGTGGCAGTCAGAGGAAAACTTGCAAGAGTTAGGCTCCC
CTTCAACTATCCAAGCTCTGAGGGTCAGACTTGGGTTGTCAGGTTTGGCAACAAGTACCTTTACTTACTAAGCCATTGTGCTGGCCCTGAATCA
GGACAGATTTAAACAAAAAGATTAACTAGGCAAACTAAATGATACCTGGAATAGTCAGATGTCCGGGCACCTTAAATTAGAAGTTGAAGCAAGA
GAAGGGACTCCAAATGTGCCTTTTCGTCCCTCTCTCTGGCTCATAGCAATCCTTTGCCACATATTTATTCATTGATAGGGTCTCACTGTAGACC
CTCTTACAGAGAGCAGGCTACCTTCGGACCTTCAGCAGTTAAGAATCCTAAGTAGTGATTTAAGTGAGAAACATCCCTCATAGGCTCATGTCCC
CAATGGCTGGTGCTGTTTGGGAAGTGTTTAGCAGCTACCCCCTTCCTGCGGGAAGTCCATCTCTGCGGAGGGATGGCCTTTGAGGGCTTGTAGC
CCCACCCTACTCTCTCTTGGCTCTCTCTGCTTCCTGTGTGCTATTGAGATATGATTCCTTCTGCTTCTTACTCCTGCCGCCATGTCTTCCCAAC
CAGTGTGGACAGCCAGCCCTCTGGAAATGTTAAGCTAAAATATAATATTTTAAATTGCCTTTGGTCATGTTATTTTATCACAGAAAAAACAAAG
TAGAAATTAATACACTGATCCTCCTGCGTCTGCCTCCCAAGTGTTGGGATTACTGGCTTGTGTCACCATATCAGCTCATCTTTATGAATCTCTG
ATTTTTGTTTTCAGTACAAGGGTTGAAGCTAGGGCCTCACAAGCAGTAGCTATGCTCTCTCCCATTGGGCTGTGTTTCCAACTCCATATTTTTT
GTTATTGTTGTTGTTTTTGTTTTTTGTTTTTCGAGGCAGGGTTTTTCTGTATAGCCCTGGCTGTCCTGGAACTCACTTTGTAGACCAGGCTGGC
CTTGAACAGAAATCCACCTGCCTCTGCCTCCCGAGTGCTGGGATTAAAGGCATGCGCCACCACGCCCAGCTCCAACCCCATATTATTAACCCCA
CTGATTCAATCATTCATCATTCATCATTCATTCATTCATTTGACTTTTCTATAATGTTTTATAAGAAGCTGCATCATTTCTTCCTTCCAGGCCT
CTCTCTCTCTCAAGCCAGGCTCACACCATAGATCTTGGTGGCAGAAGGTCTTGTCCAAAGACTGCGAGTCACACCTGTAGTGAATCCCAAAGCC
CCTTGAAGCCATGACATCATCCAGCCCAACATCTGTATGCTGACATCTAATCAGTCAACATGCTGGGACATCTTCACCAATAAGTACTGTAAAA
GAACAGAAAGTCTTTTAAATAAATGTAAAAGATGGATATTTAAGGGAGGAGGGGCAGGAGAGATAGCTCAGCGGTTAAGAGCCCTGACTGCTCT
TCCAACAGTCCTGAGTTTAATTCCCAGCAACCACATGGTGGCTCACAACTATCTGTAATGGGATCCGATGCCATCCTCTGGTGTGTCTGAAGAC
AGCGATGGTGTACCCACATACATGAAATAAATGGATAAATAAGTAGGCTGGGATGCAGCCCCGGTGATAGATGCCTGCCTACTGTGCACGAAGC
CCTGGGCTCATTCCCTCGCTACACATAAAGTCTGTGGTGGTGGCGCATTCCTGAAATCTCAGCACCAGGGAGCTCACCGTTGTCCTCAGCTACA
CGGCAAGCTTGAGGCTGGTCTGGGCACTGTAGGAGCCTTGTCTGACTGAAAAGTTATAAAAATAACTTTTCAATTAAATAAACATGCAGGGAAA
AAAGTCAAAATTCTTCACTTAAACAAAGCTACCAATGGAGATGGATAAACTGCTCAGTGACTAGAAACATCTCTCCTCTTGTATAAGGGCCCAA
GTCGAGCACACACACACACACACACACACACACACACACCCTTGAAAATAATAATGGTATCATGGTGACACACACCTTTAATCCCAGCAGTCGG
GATGCAGAGGCAGGTGAATATCTATGTTCAAGGCAAGCCTGGCTTTCACAGTGAGTTCCAAGATAACCAGAATTACATGGTAATAGCCTGTCTC
AAAAAAAAAATAGCAGACTTTATTAATATACATATATATGTATATGTGTATGTGTGTGTGTGTGTGTGTGTGTGTGTGTGTGTGTGTATGTGTG
TGTGTGTGTGTGTGTATGTATGTTTGTCTTCACAGTGCCCTCAATCCACATGTACTTCACAAGAATGTGTGTGGTGCCGTGCTTGCCGTGCACA
GACAGGGGCTGTGCTCACCGTGTGCAGGGGCCAGCCAACCTAACAGGCAAGTTTACTAGCTTCAAGCTGCTTCCAACAGAAGTGTTCAAACTCC
CTTTCTAACGATGTATTTCTAAGCTGTGTGTGTGGTGGTGCACACCTTTTTTCCCAGCACTCTGGAGGCAGAGGCAGACAGATCTCTGTGAGTT
TGAGGCCAGCCTGATCTACAGATTAGTACAGTGAAACTGGAGTCAGTGGTGGCTGTGGGTGNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNTAGGCGCTACCATTTAACCACCCCTCCAGCTCCTTGTCCATTTGGTCTTTTTTTTTTTTTTTTTTTTTTTTTT
TTTTGGGTTTCCGAGAACAGGGTTTCTCTGTATAGCCCTGGCTGTCCTGGAACTCACTTTGTAGAACCAGGCTGGCCTCGAACTCAAAAATCCG
CCTGCCTCTGCCTCCAAAGTGCTGGGATTAAAGGCGTGCGCCACCACGCCTGGCTCATTTGGTCTTTTATTCACTAGTCTACAATGACTTACTT
ATTGATTGGGTCAAGCACTCATTAAAATGCTAGAATTACATGAATGAACTAATGAAGTTAACTCGTCGAGAGTTTCTCAACTAATATGTGTTCA
ATCCATGGTCCTTTTTCTTATTTCCTTCTTATAGCACTGGGATCAAGCCCAGGGACTTGCCCATGCTAGTTAGATGCTAAGCTACAGTCTCAGC
CCAAAACACTAAGCTATGTTCCCAATTCTTTACTCTGTCAGCAGCATGGATTAAGAGCGCTCTGCAAGGCTCTTAGGAGGGTTTCTGTGAGTTT
GAGACCTGCCTGGGTATACTGGGGCTACATAGTGAGACCCTATAGCAAAAAAAAGGGGGGGGGAATGTTAAAGGGTAAACAACAAAACAGAAAG
CAAGTCCTGCATGCAGCACGCTGTACCACGCCCAGAAAAGTAGAAGGGGTGCTCTTGGTTTACTGACCAGAACAAAGCCACACACTAAACGGCT
ACATGATGACACATGAGAGATGACACTGACTGTACCATATGCTGTGACTCTGACTCACCAAAGGAACATGACTCATAATGTCTTCATCCAATTG
TTCATCAGCCAGGCTGCTCTCTCTCTGTCTCTGTCTGTCTCTCTCTCTCTCTCTGTCTCTGTCTGTCTCTCTCTGTCTGTCTCTCTCTCTCTGT
CTCTCTCTCTCTCTCTCTCTCTCTCTCTGTTTGTCTCTCTCTTTCTTTGGTTTTTCAAGACAGGGTTTCTCTGTGTAGCCCTGGCTGTCCTGGA
ACTCACTCTGTAGACCAGGCTGGCCTCAAACTCAGAAATCCGCCTGCTTCTGCCTGCTGGGATTATAGGCGTGCACCACAACTGCCTGGTTCAG
TCTGGTCTCTTAAGAATCCAGTTTTGTTTTGTTTTGTTTTTAAGATTTATTTACTTATTTTATGTATGCAAATACACCATCATTCTTTTCAGAC
ACACCAGAAAAGGCCATCAGATCCCATTATAGATGGTTGTGAGCCACCATGTGGTTGCTAGGATTTGAACTCAGGACCTCTGGAAGAGCAGTTG
GTGGTCTTAACTGCTGAGCCATCTCTCCAGCCTCCAAGAACCCAGTTTCTTTTCTTCAAATGCCTCATTGCAGTTTGTTATTGGATAGCAATAG
AGAGTCAGTTTCTCCCTTCCAACACTTCAAGCTTAAAAGATCATATGTACATATGTTGATCAGCCTATCATCCCCCCAAAATATAAGCAGTATA
TTTGACCATGGCCAAATTCCATTTCAAAGTATGTCCTTCCTGGTTAGGCTTGCAGTAGCCATTCAGGAAACTAGATGAGGGGTGTACTTATTAC
AGGCCACATAGGCTCTATGTAGTCATAACCAGACAAGTGACTGCCGTCATGTTTACAGAATAAAGCTCTAGTTCAGACTCTCTTAACTTTCAAC
TATGAGCCGGGCGTGGTGGCATACTCCTTTAATCCCAGCACTCGGGAGGCAGAGGCAGGCAGATTTCTGAGTTCGAGGCCAGCCTGGTCTACAG
AGTGAGTTTCAGGACAGCCAGGGCTACACAGAGAAACCCTGTCTTGAAAAACCAAAAAAAAAAAAAAAAAAAAAAAACCAAAAAAACCTTTCAA
CTATGAAAACCAAACTATGATGAAAACCAAAGGTGATTTTTAAAGCCAACTGCCATTTAATAATAAATAATATTCATGCCTTTATGCGATGCGT
TAGGGTTTAAGCCTTTAAATAGCATAGATCTGACCAGACATGTTTAAGAAATGTCAACAGAGCCTGGTGATGGTGCTCACCTTTGACAGAGGCA
GGCAGATCTCTGAGTTCGAGGCCAGCCTGGTCTATAGAGTTTCCAGGATGTCGAGGGATACAAAGGAGAAACTCTGTCTCAGAAAACAAACAAG
AGAAAAGTGAGGGGAGACAATGACTATTGTCATTCAGGCGTTATTTTTTTTAATCACACGGACTTACTTTTGAAATCACATAAGGTCCCCAGGC
TTGGGAACAAACCGCTAGTCAGGCTTTGCTGGGTTTTAGATGGACTCACAACTTTCTAAATCTAAACCTACTAACAAAGAAACTACACACTTCC
TCTCACAAAGTCATTTTACAACCAAAGTCTCTCTCTCTCTCTCTTCCCACTTCCTCCCTCTCCCCTCTCCCCTTTTGAGACAGTTCTCATTGTG
TGACCCAGGCTGTGAACTCCCAGGTTCAAGCAATACTCCTGAGTAGCTAGGAAGACAGGTGTGCACTGCCATGCCAGCTTTCTGTTTGAGTACT
GGTTTGTTTGTTTGTTTGTTTTAAAGATTTATTTATTATGTATACATCGTTCTGCCTGCATGTATGCCTGCAGGCCAGAAGAGGGCGTCAGGTC
TTATCACAGATGGTTGTGAGCCATCATGTGGTTGCTGGGAATTGAACTCAGGACCCCTGGAAGAGCAGTCAGTGCTCTTAACCGCTGAGCCATC
TCTCCAGNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNTTAAACGCTGCCCCGTTTCCGCAGCCGTCTGCTGGGCGAGCGGG
AGACCGACCGGACACCCCTGGGGGACCCTCTCGGATCACCGCGCTTCTCCTGCGGGCCTCCAGGTAAGGGACAGCTCGCCGACGTCGTTTTGCA
TTTGAGAGCTATTGCCGAAAGACGTTTTCTGTCTCCTTCGTAAATGCATGTATCTATTTGTCCCTTTTCGTAGATGTTTTATAATTTCAAGTTT
TTATCGTGTGTGTTGTTGTTTTTTTTCTACTTGTAGGCCAATGTGCAATACCAACATGTCTGTGTCTACCGAGGGTGCTGCAAGCACCTCACAG
ATTCCAGCTTCGGAACAAGAGACTCTGGTTGGTATTTCTGCCTCGAGTGTGTAAATATAGCTTGTTTTACCAGGTAATCGAGGAAACAAAATTG
AAACCTGGATTATCGCACAGTCGTTTTAAGTATTTACCTACAAACTCTTAACTTTCTCGATTGCAATTGCATTAGAGAGTGGTCACTGCGACCT
ATATTTCCACAGCCCTTCCCCTGACGCAGGCTTTAGAAGTGCACAGTGTGTGGGTGCTGCTGGTTGTGACATGATGTTCTATCACTGTTCTAAA
TGGTAAACCGAAAGTCATAGAAGCATCCGTGCTTTTTGTGAGGATTTTAGTTATGAGAGGAATGCATAAAATATATAAAGTTCCAGTCTTTGCT
CTGTATCTCTTTAAGTTTTCTACTTTATATTAGCATATAAAGGTGTTCTACAGCTTGTCAGTAAGTTACAAAATTACTTTTAATAGCTGCATAG
GATTTGATAATGCGATTGAACCTCAGCCTTTAGAATGTGTGTGTATGCAAGTACTGTACTACTGAGCTGAGCGACAGTCTCAGGCTCACGACTA
CTGGGGGATGTAAAAGTTGCCCATGTGTTTGAGTAAAGTAAGGGTGAGGAAGATAGGGAACTAGAATCCTACGTCCTGGTAACCACATTTGATT
GCAGACTGCTCACTCTGCTCAGCAGAGGTCATGGCCGTCTTCGATCCTGCCAGCACGAATATTGCCAGCCCTCGTTTTCCCTTTCTGATGTAAT
TTCATACATATTTGTGGAGAATACATGTATGCAAGTTAAGTCAGTTCCTACGGTTACTGCTCTGTGGAGTAAAATGTATTGTTTCAGTGGGCAT
GTCCCAGATCTCCAGTTAAACAGCCTGTTTGTTCTGGGTTTGCCAATGTCTCTCTCTATTTACTCTCGCTCTTCCTTTCCTCCCCCATTTTTTT
TTTTGCATATTTTAGTAGCTTGCATAATTTTCAAGTATAGTTAACCTAAGAAATTTTTTTTTACAAGGACTATAATCTGATGTCTGCCCCATTG
AAGGTATCAAATTTGTTTATTTTGATAATTTTTGAATTTTCTTGTAATGCTTTGGAGGAATTCTTCCATTTCAAGATTATGCACATATTTAAAG
TATTTTCATATAGCATTTTAAAAACCTTTTCCGGGTAGATCTTTATTCAGAACTCTCTTTGTTAAGTGTGAGATTACAGTGTTGTGTTTTTATA
GTAGTTTTTAAAATTAACTTTATCTTGTAAGCTTTGATAAGAAACTGGCTTTCCATTGTAGAGTTTTGATTTGAGTTAATTTAATATGCAAAAA
TGTAGAACTTCTTGCTTTGGCAGTACGTATGTTAAAATTGATACACTAAGGAGAAGGCTAGCATGACTTCTATATGAGTTACATAAGAAAATTA
AGCATTAGTAAGTCTTTTTCAGCTGGGCGTGGTGGCGCATGCCTTTAATCCCAGCACTTGGGAGGCAGAGGCAGGCAGATCTCTGAGTTCGAGG
CCAGCCTGGTCTACAAAGTGAGTTCCAGGACAGCCAAGGCTACACAGAGAAACCCTGTCTAGAAAAAACAAAGAAACAAAAAGTCTTTTTCAAA
TCTTGTAAGAGTGAATTACTTAATCTGAAAATAAGGTCTTAATACCCTTTTTTAAAAAACTGATTTCTATAGGCTGGAGAGATGGCTCAGAGGT
TAAGAGCACTGACTGCTCTTCCAGAGGTTCTGAGTTCAACTCCCAGCAACTACATGGTGGCTCACAACTGTCTGATGCCCTCCCTCTTCTGGTG
TGCAGGTGTACACGCAGACAGAGCACTCATACACAGAATACATAAATAACAGAGTCTTTAAAACTTTTTTCTTTATTTTTGAAATTATACCATT
TCTCCTTTCCTTTTCCTTCTTCCAAAGCCTCCAGTATACCCCTTCTCTCTTTCATGACTTTTTTCATTAATTGTTACATGCCCAGTACATGCTA
CGTTCCCAGATACAGCCTGCTCAGTCTGAATAATGCCATTTGTGTATGTATGTTTTCAGGGCTGACCTTTTGGTATTGGATAACCAGTTTGTGT
GCTCTTCTGGAATAAATAACTGAAAAGTGCATTTTAAAATTGTGCTAAAAGTAGTCTTTAAAAAAAGAAAAAGAAAAGAGTGGTGCTGGAGAGA
GGGCTCAGCAGTTAAGTGCATTTGCTGTTGTTACAGAGGACCCAGGTTGGATCCCCAGAATGGATAAGGTGGCTCAGCCCTCTTGTAACTCTAG
TTGCAGAGGATCTGATGGCCTCTTTTGGCCTCCAAGGACACTGTTTGCACACGGTGCACTTAAATACATCAGGTGAAATACCAGTATACATAAA
ATAATAATAAAAGTCTTCAAATTACCAAAAAAATCCACAGAAGTGGTGGTCTGTGTGGTTATCTCAAACTAAAACAGCTGAGTTTGCAGATACA
GAGAGCATAGTCATGTTACATGCCTTCCTTCCCAGTTTCTAAACACTCTCTCAGCTTAGTCAGCTCAGTGTACTTACTGTCCTTGAAGCACTTA
ATAGTATTTTAAAAATAAACTAAATGCCTTTAAAGGTAGTGGTTAGTCCTATTTGTCCTCAGTACTCTGCCATGAGGCCACATTCTAGCCATGT
AGACAGAGGCCTGGCACAGTGAGATCTGCAGCCATTGCAACAATAGAGCCATACAAAATAACTGTCCTGACAGAGACAGCCGGCCAAGCAGCCA
GGAGGGAGCACGACTGCTCTTCAGAGGACCTGGCTTCCACTCTCAGTACCCACATGGTGGCTCCTAGCCATCTGGAACTCCAATTCCAGGGGTC
CAGCACTGTTGTGGTATCCCTGGGGACCAGGGACACAAGTGGTGCATGGAAGTACATGCAGGCAAAACATGCATATGCATGGAACAGACAGAAA
GCCAGCAGGCTCTCCGTGCTGGGCATCCACTAGCTATAGAACACGAGTGAACAGGAGCCTTGATCATCAAAGCTGACGTGCAGGAGTGTCTTTA
GAGTATCTGTGGCCGATGGTAACATACTTTGATATCAGACTGACGTGGCTAGAGCAAGCTCAGGGACATTTATCATGGTGTTCCTTGGTATCCG
TTGTCAGGCTGCCAGGCTGGGCTCACTAGCTGCTTTGTGGGTGACACCTTGTTAGGTAGGTGACAGTTGTCCAGTTTTTATCTTTTTAAGGATT
CATTTCTTATTTTATATATAGGAATGTTTTGCCTGCATGTGTATATGTGTACCATGTGGATGTCTGGTGGCTATAGAAACCAGAAGAGGGTGTC
GGATCCCTTGGAACCAGAGTTAGATAGTTTTGATCTATCATATAGGTTCTGAGAACTAAATTCAGGGTCTCTATAAGAGCAACAAGTGGTCTTA
ACCTCTAAGCCTCAGCCCCATCAACTATTGTCTCACTACTGGGAAAGCTGGATTAGAGTCAAATTGTAGCTGGCTACTTGTTTTTATAAATAAA
GCTTAATGTTATATATTTACTCTTATAGTTTATGTAGTACTTTTGCTTTTAAGTTGGCTAGACATAGAAAAAACACTTTCTGGCTCTTCTGATA
TTACTGGCATGCTCTTTAAAGATCATTTGTCATTAGTGAGTGTGTGTGTGTGTGTGTGTGTGTGTGTGTGTGTGTGTGTGTGTGTGTGAGAGAG
AGAGAGAGAGAGAGAGAGAGAGAATATCTAGCCTTTGCATGTATTTTAAGTGATAAATAAATGATGCATAAACCACATATAAAGAAAACAATGA
GTTGTAGCTGAGGGCTGTGATTGTACTCTTTGAATTGTTCATTACCTTGTGACTTTGGTTTCTTTAGCAGTCAGGAATGTGGTATATGATAGAT
TGGTGATAGGTTTTGCCCTAAGATATAGTCTATTCTCCTACATTCTATAAGCTCCCTTTCAGTAGACTGACGGATTTGTACAGTATAGTAAGAG
ACTGGATCTCAGCTAGGTTGTGTGGTGGCAAGCAGATGATGGAGCTGTAGGAAGGAGTCAGTGCTCACTCACACACTCCCCTGTGTGTGTGTGT
GTGTGTGTGTGTGTATTTGTAGCTCCTGCCTGCAGAGACTACAGTGGATGTTGGATTGCCTAGGATGTTGGTAGCTGCCTTTTGGTGGCCCTAG
GAATCTAGAATTGGGTCCCCTGCAAAGCAGCAAGTGCTTTTAATCTCTCAGCCATCTCTCCAGCATCTTGATACCGTTTTGATCAGAACAAATT
GAGTTTGATTATTTGACATGACACCAGGATTTGATGACTTCATGATAATTTCATAGTAGCTTTTCCCAAAGCATTTTCTGTCCTAACGCTTAGT
GGTGTGCACTTGTACTTAGTGTAAAACCCAACTTTCTGAAGTGCAGCTCGGTAACAAATTGTTATAAGCACGGCGTTTGCTGCCTGTATTGGGC
TTCTTTCTGGCCATACATAAAGGGCTTGTGCTGTAGAGTATGTTCATGGTGTAACATCAGCTCTTTTTCTTTTCTTTTACAGGTTAGACCAAAA
CCATTGCTTTTGAAGTTGTTAAAGTCCGTTGGAGCGCAAAACGACACTTACACTATGAAAGAGGTGAGCAGAACTGAGGGGTGGAGAGCCGATG
GCTTCATGGTTACATACAGGCATCTGTGGTGGTTTTTCAAGTTTGAGTGAGATTGGCCATACAGATTTTGTAATGAGCAAGCACATTAAGTTTT
GTGATCATTTGAACTGTAATTTGAAGCTAAAAATTCTAATTTACATGAATTTCTTCCTCCTTTCCTTTCTTTGAGACAAGGTCTTTTTTTTAAA
AAGATGTATTTATTTATTATATGTGAGTACACTGTACCTGTCTTCAGACACTCCAGAAGAGGGCATCAGATCTCATTACAGATGGTTGCGAGCT
ACCATGTGGTTGCTGGGATTTGAACCCAAGACCTTCAGAAGAGCAATCAGTACTCTTAACCAGAGCCATCACTAGCCCCTGAGACAAAGTTTTA
CTATGTAGTCCTGACTGTCCTGGAACTCACTATGTAGACCAGCTGGCCTCAGACTCAAGAGAGATCCACCTGCCTCTGCCTCCTGAGTTCTCTT
TTCTCTTTTTNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNAAAAAGAAAGCAAAAGAAAAAAGACCTTATACAACTGTTGGGATGCTCAGTGGGCTGATTCATAGACACAATGTTGTATTCTCTGCCTT
AACGTTTATTAGTAAAGTATTGTTTTCAGCTAGAGGTTGGTCAGTTGTAGACAGAAGTTTTGCATGGCTGAGATATAAGTGTTTCTCTGGCTCA
CTTAGGTTAAGGCTTGTTTACTTAAAACACTCAAGTTCTTGTGGCAGGTTAGGGGCTTCTTCTCAGCTACACATTTGGAAACGTCTCAAAGAAC
ACTTTGTTGGAGGAACCCAAGTGTCCTTTCTGTCCCACTGACTGTCAATACTTTGTGATCTATAGACATGTAAAAATTGCCTTTCAGATTGATT
GGCTCCTACTTAGCTAGTTAAAGTCTCTTTAAGTGTTAAGTGATGTTTTTAAGGTCATTAACTTTGGATTGACCTTTATAAAGGTTAGACAAAT
GATAAACAAGTATGGATTTAGTAGTAGGTAGATATGAGAAAATCGATGAAGGCCATCAAAAAGATTTAGTGCTTATGCTAAGCTGTGTGCAGTA
GGTCAGCTGTCCTCCTGCTCTCTGCGGGGAGTTGGCTTCAGGACACTGCCTGCCAAGTCTCTTACTTAAACCGTGAAACTGTCTCTGTATGGAC
ATCACCTCTTAGACATTAAGTTATCCCTAGAGTATCTGTAATACACAATGTGTGTAGTTGTTATACTGTATTCTTTAAGGAATAGTGACAAGGA
AAAACATTTCTACATTCAGGATGGATGCCAGGAATATTATTAGACCCACAGTCAAATGGATCTGTGGGGGTGGGACTTACAGATTGTTTATAGT
GTCTGATACCAGTTTTCAGTTACAAGATCTTTATTCTAGGACACTTGAGAATGTAGTGAGCTAGTGTGGTAGACAGTACGCTGGGATAGGTGTC
ATGTCCTGGGTTAGTTTCTCATCTCTGTTTATGTTTTTATTTTCCCCCCTGTGGTACTGGGTGTCAGATCAAGGGAGCACATTTTACCTACTGA
ACGACACTCGTGACCTTGTTTTCATACAGGAATAACAGTTCCCTGGGGTGTCAGGATGGTGAGACTGTACTGACAAACCTGTGCACTGTAAAAT
GCTGTCTAGAGTTACATTGTTGATGGGCTCGTTTTGGAATGACAAAGTATGTTGCTTAAGCTGCCATAATGGAGCACATGGAATATAATAATAG
TGATGGTTCGGGCCCTGGACTGTTTGGTGAAGACTATATTAATAACTGAGAATATTCCATTGGGAGATGTTACTCATGATGTCCTGTCTTCCTC
TGTGATGACCTCAGAGCTGTAGAAGTATACGTTTGTGACATGTCACTATTCTAATACATTGCTGTATTTTACCTTACTCTTTTTTTTTATTCAA
TTTTTTTTTAAGATTTATTTATTTATTACATGTAAGTACACTGTAGTTATCTTCAGACACCCCAGAAGAGGGCATCAGATCTCATTACAGAGCC
ACCATGTGGTTGCTGGGATTTGAACTCACAACCTTTGGAAGAGCAATCAGTGTTCTTAACCACTGGGCCATCTCTCCAGTGTCCCCCCCCCCCC
TTTTTTTTAAACCTCTTTTTTCTTTCCTAGTATCATATGTTAGTATCTAAGTTTCTGGGAAAAATTAAAATTTATAAGAAAAAAAGAAAAACAG
AAAATACATTTAAGTAATGTGGAGAAACAGTTACTTCTATTTTCATCAGTGTGGAAGTATTTAAAAGAAGCTTCTACAATACAACTGTTTGGTC
TTGTACATGATTATGGGTGTGGCTGTTAGTGTAAAGTGTAACTTGTTCAGTTAAGTGTGGTATTGCTGAATGTATAATGTATATATTCTATGTG
ATTTCCTTAGTACTTAGAAACCATATTTGTGTTTCAGATTATATTTTATATTGGCCAGTATATTATGACTAAGAGGTTATATGACGAGAAGCAG
CAGCACATTGTGTATTGTTCAAATGATCTCCTAGGAGATGTGTTTGGAGTCCCGAGTTTCTCTGTGAAGGAGCACAGGTAAGCCTTGTGCGTGG
TGGGTTATAAGAACAGCGAGCGGCTGTGCATGGGGCTGGAGAGATGCTCAGTGGTTAAGAGGACCCAGGTTCAATTCCCAGCGCCCCCATGGCA
GCTTACAACTGTTGGTACTGCCAGTTCTATAGGATCTGGTGCCCTCACACAGACATACATGCAGGTAAACATAAAAAACATCTTAAAGAAGTAC
CAGCTGGGCGGTCACTGTTTCTCACCTCTGTCAGAGAGGAACTGACCATGGATACAAAATGCTAACATTGGGTGTCATTTTTTGAGGCCCTACT
GCGATTAACAGTAGTCGTAGCATATTGTGTGGGAACCTGAACTCTGGAATCTTAGTTTTGATTGTGTGTGTGTGTATGTGCATATGCCACTGTG
AGTGTGTGGCAGTCAGGAGTCAGCTTGTCCTTCCACCATGTGGGTACTGGGGATTGAATGCAGGTTACCAGGCTTGACCTCTTTTGAGACAGGT
CGAGTCGTTAGTCTGTGGGTGTGTAAATGGATCTGACATCTCTCAGCTGTAGGCTCTCTACTCACATGTTGTTTTTTTTTTTTTTTTTTCTTTT
CTTACAAGGAAAATATATGCAATGATCTACAGAAATTTAGTGGCTGTAAGTCAGCAAGGTAGGTAATTTTGAATATTATAAACATAGTATAACT
TAATAACAGTTCTTTTTCATGTAAGATCTTATGTATCCTAGTCTAACCTTAAACTATGTAGCTACGGACCCTTGAGCCTTAAAAAAAAAAAAAA
AACCACAAACCTTTGAGTAGCTTTTTGTTGTGTATGTATCTGTATGTCTGTGTAGGGTGGGATAGATACATGGTGTATACCTGGAAGTCAAAAA
ACAGCTTGGCGGAATTGGTCTACTTTTTACATGGGTTCTGGTATCCAGCTCAGATCTGTAGGCGTGTGTGGCAGGCACTTTGACCTCCTGAAAC
ATCTTACCAGTCCCCTAATCATGTTTTAAAGACTTGGATGTTGTCTTAGTCAGGGTTTCTATTCCTGCACAAACATCATGACCAAGAAGCAAAT
TGGGAAGGAAAGGGTTTATTCGGCTTACACTTCCATACTGCTGTTGACCACCAAAGGATGCAGGACTGGAACTCAAGCAAGTCAGAAAGCAGGA
GCTGATGCAGAGGCCATGGAGGGATGTTCTTTACTGACTTGCTTCCCCTGGCTTGCTCAGCCTGCTCTCTTAAAGAACTAAGACTACCAGCCCA
GGGATGGCACCACCCACAAGGGGCCTTTCCCTCTTGATCACTATATTGAGAAAATGCCTTACAGTTGTATCTCATGGAGGCATTTCCTCAACTG
AAGCTCCTTTCTCTGTGATAACTCCAGCTGTGTCAAGTTGACACAAAACTAGCCAGTACAGATGTATACTTCATGTTTTAAAGACTTGGATGTA
TACTTCTTCGTAGAGTGCAGTTCTGGCTTTAACCCTTAGCACTCTAAAAAGGCAAAATATCACTGTGTGCTGCTTTCATTTGTGTTTGCTTCTG
GCTCTCTTTATTATTTTTAACTCTTTCATTGCCTACCAATTTTTTTTTAATAATTATTTTTGGGTGTATTAGTGTTTTGCCTGCATGTCTGTCT
GTGTACCATCTGTGTGCAGTGCCTTTGGAGGACAGAAGAGCCCATCAGGTCTCCTAGAACTTGTTACAGACAGTTAACGACTGCTGTGTGGGCT
GGGAATTGAAGCCAGAACTTCTGGTAGAGTAGCCAGGGCTCTTAACCATGAAGCTGTGTTCCCAGCCTCTACTTTTTTGTTGTTTTGAGACCAG
ATCCCCATAAATTATCCAGGCTCATTTGGAACCCAGGCTTCCTGCTCCCCAAGTAGCTGACGTTTGTTCATTTATTCAGCACTGCTGAGAGTTG
AACTTAGGCTCACACACCCTTGCGTTTAGAAAGTACTGTACTAGTTAGCTGCCCCAGCCAGGCTTTGACTCATCTCATTCTCTCTTAAAAAATT
AAAGTGACTTCTTTCATTTCACTTTATTTGTATGGGTGTTTTTTCTGTCCTGCTGTATGCATGTATACATGTGCACCACGCGTGTGCAGTGCCC
AGCAAGGTCAGAGGAGGAGCATTGAATCTCCTAGGACTGGAATTAGACTATTGCCACCTCTGGAAGACCTTCAAGTGCTCTTAAGCACCAGAGT
CATCTCTCCAGCCCCTCTTTGGAAAAAATAATTATACTGCCTGGTGTGGTTACACAGACATTTAATCTCAGCAAATGGGCGGATGGACTTGTCC
AGCCAAGTTTACATAAAGAGACCCTGTCTCAAAAAGGCAAAAAGCAAGTTTTAATTATGTGTGCGTCCCTTTATGTGAATATGTGCACATGAGT
GCAACTGATCTTAGAGGCCAGGAGTGTCAGCTTCCCTAGAGCTGGAGTTAGGGAGTTTTGAGCTGTGGTATCTAGGAACTAAGAGTAATCCAGC
CCCATGATTCTCCTTAAAAGTAAAAGAAAGTACATTTATTCGTGTTTAAGAGCATCCTTATGCCATGATGTGCATTGGGAAGTCAAATTGGATG
TCTTGTTAATCTCTGTCACGACCCTGGGTTATCAGGGTTTCTTCTGAAGGACAGGTAATGTTAAAAGCTGCTTAGTAGAAGCGTGATCAAACCT
CAGAATGCGTTGAGATGTTTCAGGAAGTAATATAATACCTAGGTAGGACCGTGAAAGAGTGGGGTTTCATCTCTTCTCTTCTCTTGCCCCCCTT
TGTATTCTTGCTCTTGTTTCTTGAGTGGACACTTAGCATTTTTAGGTAAGCATCATTTTAGCTCTCCTTGGCAGTTCTTTTTTAGTGACTTGAG
CACAGGTCCTTGTGTGTGCTTCACAAGCATCAACTCTGATGTTGAGCTACTTACTGAGAGAATTGGTTATTTTTGGGATGCAACCTTTTTGAGA
ACCTAGTAAAGGCACTTGATTTTCATTCTATAAGTGTGTACATGTGGATAGTGTTTTATTATTATTATTATGGTTTTTTGAGACAGGGTTTCTC
TGTATAGCCCTGGCTGTCCTGGAACTCACTGTTTAGACCAGGCTTGCCTCGAACTCAGAAGTCTGCCTGCCTCTGCCTCCCAAGAGCTGGGATT
AAAGGCATGCGCCACCATGCCCGGCTATTTTTTTTTTTTTTTTTAAAGAAAGAGTTTCTCTTTTTAAAGACAGGCTCTCACTATTAGCTATGGT
TGGGCTAGAACTCATTATGTAGATCAGGCAGACCTTTAACTCCCAGAGATCCATCCACCTGCCTCTGCCTTCTAGACTTACATACCGCCATGTC
TCCTATTAGTTGTATTTGAATACTAGTAGCTGTGGGCTCTGAAACCCCGTAATCCTTACCGTTACCCTTGAACATAGCTACTGTGTTCAGAAGC
TTGCTTGGTTTCTAGACTGAATCAACCATTCTCTTGCGATCCCACTATTCCCTTCTATATTCAGAAGTCACTGTGTTGTGTGTACAGTGTGTTG
TAAATGGCACCAGGATCCTCCCTAAAAAAAGAACAAAGTAATTAGTTTGGTAACTGAGACCTGTGGTTTTCGGCTTCTGCCTCATGATGCTGAG
GTTACTGGGACCTGCTAAGGCTCTTTATGTCATGCTATACAGTTATGGAATCTGATAGTATTTTACATTTTCTCTAGTAGTCACTCACTTAATA
AACTTTGACAGATTACTGACGATTTCTCTTGATGCTGTGTTTCAGTTTAGTTAAAAGTAATTTTGAAAATCCTAGGTGAGCCTGTCACTCCCGT
TACTTTATTACATCTGTTTTATAACTATACACAGCCCAAGACAGAGTTCCCAGGTAGCATAGCCTATCTATGTATTCCCTTTAAAGTTTGTATT
ATGCCATAATACAGGACAGTTCACTGCGGTCACCCAGTGTTTGAGGAGCTCGGCAGTGCCCAGCTGTGGTCTCACGGTTGATGGATGTGCTTTA
GGCGGTAGTCTAGGGTATGGATTGTGATTTTTGAGTAATTTTGTCCATTCCAGTTCTGGAACTTGGATGTTTTGAGAGCTTGGTCTGTGAAACT
AAGTACAAACTACACCTGTCTATCTACATAACTCTTTTAAATTCATTATTTTTTCCAAGCTAAATCGTTCACCTCCCTCACCTCTCTTGGTGAG
TGGTACCACACATTCTTCATCTAGTTAGACTCTTGGGATCACTTCCTTTTCCTTACAGACATCTAGTCTTAACCAGTTTTTATGTCTTAATTTT
TCTCAAGTTGATTGTCTTAGTTAGGGTTTGCATTGCTGTGAAGAGACAACATGACCAAGGCAACTCCTGTTATTAAGAAAAACATTTAGTTGGG
GCTTGACTTAATACTTCAGAGGATTAGTCCATTATCCTCAAGGCAGTAAGCATGGCAGCATGCAGGCAGATGTGGTGCTGGAGGAGCTGAGAGT
TCTATACCCTTATCCAAAGGTGGAAGGCAAGTAAGAGGCAGGTTTCTTCCACATTGGGCGGGGCCTGAGCAAGGGAGCCTTAGAGCCCACGCCC
ACAGAGACACACCTCTTCCAACCAGGTCACACCTCCTCTAGCCAGGCCACACCCTGGGCCAAGCATATTCAAACCACCACTTAAATACTTTAAC
TACATGTATATATGTATTGTATGCCGACTAATGCCACANNNNNNNNNNNNNNNNNNNNTATACACACACACACATACACACACAGTTGTCTTTG
TATTTCAAATACTGTTTGCTTGTTTCTGTGCTCTGTTTGCCTTCCCGTGTGGACCTCCTACATGCTAACTGAGGCCTCCTCCACTGAATCACTA
TGGCTTGACAGTCTCACTGAGTTGCCCAGCCTGGCCTGCTGGAGATGATTGCTGTAGTGTAAATCACCGTGCCCAGTTGGGAGTGGTGGTGGGT
TTGTTGTTTTTTAAATTCTATTTTGTCAGAGTGGTTTGTTGCTTTTTTTGTTGTTGTTGTTCTTTTTTTGTTTTTTTGAGACAGGGTTTCTCTG
TGTAGCCCTGGCTGTCCTGGAACTCACTTTGTAGACCATGCTGGCCTCGAACTCAGAAATTGGCCTGCCTCTGCCTTTGCCTTCCAAGTGCTGA
GGTTAAAGGCGTGTGCCACCACCACCTGGAGGTTTGTTGCTTCTTATTTAGGAGTTTTATGATTGCTCTCTGGGGAGAATAGTTCTGTGAGCTA
GGAAATTAGCTTTATTTCTTGAACTAAAACCCTTTAATAGTGAGAAGTTGCAAGATGGCCTCAGGCAAGATTATAGAACTGCTTGAATAACTGT
TGAGAAGAATAACCTTGTATGTGACTCAGCTGAACACGCATGGCTCTGTGCACAGCGACCCTGCACTTTACTGTTTGAGTACAGAGGAGTTTAT
TATGTTGGTGTCAGTCACAGGAACTGTTTGCGTGTTCATGATTTCTCACATACTAATCCTTCAGTCTGGAGTGTCTGTCCTATGCTAACTTCAG
CGTAGGCGATTCCAGAATCTGTCCTGATAGCTCTTCAGAGCCCCTCCCCCACGTCTACTAAACATTTTTGTTTCCCCTGCTAGTATATCTTAAA
ATTTGCTTATTTTGCTTCATAATTCCTGTTGAATTTATATCTCCAGCATGCAAATGACTCTTGATACAAATGGTTAATAGTTGTTAAAAGGTTA
GATTTTGTATTGGGCATGTGTTAGACTGGTATATGTTGAATTCTTGATTTCTTTGTTTTGATCCACTTTTTACTTTCTCTTACAATCTTCAGAC
TCTGGCACATCGCTGAGTGAGAGCAGACGTCAGCCTGAAGGTGGGAGTGATCTGAAGGTAATACTTGCATAAAGTTCTTACTCTAACACACTTT
TCTCTTTAAAATATCCTGTCTGTAAGTGTGGGTGTGTGTATGTCCTCCAGCCAAAAGAAAGAGGTGATGCTCACCTGGAGCTGAGTTACAGGTG
GATGTGAGCTGCCCCAGACATGAGTGATGGAAACCAAACACACCCCAGCAAGAACAGCACATGGTCTTCTTCTCTGAGCCACCGCTCTAGCTTC
ATGACACATAGTTCTTGAAAATTGGGAGGGTTACATAAAACAGGGTGTGGAACAGCCAATGTTTAATGTTTATTTTGCAATATTTTACTTTCTT
TTATGTACTTAGGATCCTTTGCAAGCGCCACCAGAAGAGAAACCTTCATCTTCTGATTTAATTTCTAGACTCTCTACCTCATCTAGAAGGAGAT
CCATTAGTGAGACAGGTACATAGGGACATTATGTGACATACTAAACAGTTGCTTGGTGTTTCCATTTTGGGTCAAATGACCCTTTGACAGAGGT
CTGAGACCTAAGACCACTGAAAACCACATGGTTGCATTATGATTCATCACATTAGCAAAACTATGGCTATGAAGTAGCAATGAGAATAGTTTTA
TGCTTGGGATCACCACTATGAGGAACTGTATTAAAGAGTCACAGTGCCGGGCGATGGTGGCTCTCATCTTTAATCCAGCACTTGGGAGGCAGAG
GCAGGCGGATTTCTGAGTTCGAGGCCAGCCTGGTCTACAGAGTGAGTTCCAGGACAGCCAGGGGTATACAGAGAAACCCTGTTTCGAAAAACCA
AAAAAAAAGAGTCACAGTGTTAGGAAAGTTAAGAGCTGCTACCCTAGACTTCTAATAAAAGGAGATGAAGTTGTTCTTTGTTTTGGGACAGGGC
TTCTATATAGATCAGGTTGACCTTTGCATAGTCTTTTTCTTTTTTCTTTTTTTTTTTAACTTTATTTATTTAATGTATGAGTGCTCTGCTGCTC
TTAACCACTGAGCTGTTTCACCAGCCCAGCCTTATTCTTAAAGCTTACTGTTGATGGGGAAATCGTGTACTGTTGAATCAGTAAAAGCACTAGG
AGGATTAAATGAGTAACTTGTGGAGGTAACGTCCACAAAGAAGGTTGCAAGCTTAGAAGTAGAGCTTATGTGTAGCTGAACAGATCCAATGAAA
CACTGCTGACTTTTGAAACATTAATGGTTCACTTCTCTGTTTAATATAATTGAAATTAAATTTTGTTGCAAATAGGTGCTGAAGATATTTCAAT
CTCAAATCAGTCCCAGTACAGGGAGTTTTAACCCCATGTCCTTTAATCTATAGAAGAGAACACAGATGAGCTACCTGGGGAGCGGCACCGGAAG
CGCCGCAGGTCCCTGTCCTTTGATCCGAGCCTGGGTCTGTGTGAGCTGAGGGAGATGTGCAGCGGCGGCAGCAGCAGCAGTAGCAGCAGCAGCA
GCGAGTCCACAGAGACGCCCTCGCATCAGGTAGTGCTTGGGTTTAAGAAATTATCTTACTTTTTAATATGGAAGTGTCTTGTCTGCCAGAATGT
ACACCACATGCATGCAGTGCTTTGAGGCCACAGGAGGGTGTGGAGTCCCCTGGAACTGGGGTTAGGGATGGTTGGAGCCACTGTGTGGGTGCCA
GGAAAGTGGGTTGACTACCACATCTCTGCCCTAGTTTTGAGTGCTGCAGATTAAATCCAGGGCTCTGTGCCCACTGGGCAAGTGTTCTACCAGT
TAATTACATCCCCAACACCCAACAGTCAGTGAGCCAACCTCTCTGCGGATCTCTGCCTATCTAGTTTCTTAGAATAGAACCTGACCTGCTGTCA
AAACAGAATATATATATATATATATATATATATATATATATATATATATATATATATGCTCTTCTAAAAATCTTCCATATTAAAAATTTGTTAC
TTTTTAAACTTAAGTGAAAATGCTTTTAATACAACTGTGTTTTGCTTTGTTTTAACTTACTTATGAGAACATTAGTCTCAGTAGTTTTACCCTT
TATTTACAGGGAACACTATGCCTGATAGTGTTTCAAATAATTTGTTAAAGGATCCTACAACTAATTATTACATTTAATAAGACAGGCTAGAACT
TGAAAGCTCAGCGGTTAAGAGCCCTGACTGCTCTTCCAGAGGTCCTGAGTTCAACTCCCAGCAACCACATGGTCGCTCACAACCATCTATAATG
GGATCTGGTGCCCTCTTCTGGTGTGTCTGAAGACAGCAACAGTATACTTAGATATAATAATAAATTAATCTTAAAAAAAAAAGACTACTTTTAT
GTCAAGCTGACTGTATTTTCTGGAAGAGAAATGATGTGTCTGCAGTTAAGCAGCATCTTGGTAGCAAATTGAGAATGCCTGTGTCCTGAATTCT
AGAGCAGTGTATTTTTATCAGTTGTAAGTAAGTTGATTTGCGGATGAACTTAGTGTATGATTTGAAGTTGAATCTCTTGTTTGCATTTTTGTGA
GACAGGGTCTCTTATTTAGTTCTGACTCTTGGCTGTCCTGGAACTCACTGTATAGACCAGGCTGGTTTTGAACCTACAGAGATCTTCCAGTCTC
TGCCTCCTGAATGAATGCTAGGATTAAAGGCATACACCACATGCCTGGCCTGAAGTTGAATCTTTTAATCTCAGCACTCAGGAAGCAGGGACAG
CAGAGCTCTCTGAGGTAATAGCCAATATAGTGTCACATGAGGTAATAGCCAATATAGTGTCACATGAGGTAATAGCCAATATAGTGTCACATGA
GGTAATAGCCAATATAGTCTCACATGAGGTAATAGCCAATATAGTGTCACATGAGGTAATAGCCAATATAGTGTCACATCAGGTAATAGCCAAT
ATAGTGTCACATGAGGTAATAGCCAATATAGTGTCACATGAGGTAATAGCCAATATAGTGTCACATGAGGTAATAGCCAATATAGTGTCACATG
AGGTAATAGCCAATATAGTGTCACATGAGGTAATAGCCAATATAGTGTCACATGAGGTAATAGCCAATATAGTGTCACATGAGGTAATAGCCAA
TATAGTGTCACATGAGGCCCCAGGCCAACTAAGGCTGCATCGGAAAACCCAGATGCCAGCTTCGTGTTTGTGTCTCGTCACTAGCACTCCGTCA
CACTAGCCTGTGAGTCAGGGAGGCCTTCGTCTTCCTGTGTTTAATAGGATAAACTCACAGTGGTGCTTCAGATAATAGTGGGTGTGCATATAAT
CACACACGTAGGAAAGGTCATTGTGGAAGATCTTTTTATGGTATTGTGGGAACCTTAACCAAAGAAAGATGAGTGGGGCTTAGAAACTCAGATT
ATCTTACTGAGAAAAAGTTAGTGTGTGGATTGATTTAGGAAACAAGATGTGGCTGGAGCATGGGTATTGAATAGGGTGGAGGGTGTGTTTAGCT
GGACATTTGAAAATGTTAATGCAGTCATTCTGAGTGTAACCTCGGGGCTAAGGGGCTCTGACTTTTCCTTCATGAGGTAGGACTTTTGAGTAAG
GTAGTAACCTTGAACCCAGTCTATGTGGTCTGGGGCTGAGAAGGAAATAGTGGCTCGGGATGAACCCGGGAGCTCTAAATGAGGCCCAGAGGCA
ATGCATTTACATTCGCTGCATTCTTCCTCCATAGGATCTTGACGATGGCGTAAGTGAGCATTCTGGTGATTGCCTGGATCAGGATTCAGTTTCT
GATCAGTTTAGCGTGGAATTTGAAGTTGAGTCTCTGGACTCGGAAGATTACAGCCTGAGTGACGAAGGGCACGAGCTCTCAGATGAGGATGATG
AGGTGGGTTTCTGTGTCTGTCAGCCTTTTAGTAAGATGAAGAGGCCATCTATTCAGAGTCTCTGGAAAGTCTTGCTTTTTCATTTGTTCAAGAT
AGAATATTGCTTTGTGGATTTGAGGTGAAATTTTATAGTTTCTTTGCTTGATTTATTTATTTAGATTTATTTGTTATGTATACAGCATTCTGCC
TGCATGTATGTCTGCAGGCCAGAAGAGGGCACTACATATGCTTACTAGCCACCATGTGGTTGCTGGGAATTGAACTCAGGACCTTTGGAAGAAC
AGACAGTGCTCTTAACCTCTGAGTCATCTCTCCAGCCCTTTGCTTGATTTATTATAGTCTCACATTACTAAATAAAAAACTTAGAGGAGAGTTT
GAACAATGTTTTTAAACTTTTTTTGAGTTGATGGGGTGTTTTTGTTTTGTTTGTTTTGCTTTTTGAGACTTGGTTTTTTTTTTTTGTTTTTGTT
TTTGTTTTTGTTTTTTTTTTTGGTTTTTCGAGACAGGGTTTCTCTGTGTAGCCGTGGCTGTCCTGGAACTCACTTTGTAGACCAGGCTGGCCTC
GAACTCAGAAATCCACCTGCCTCTGCCTCCCAAGTGCTGGGATTAAAGGCCTTCGCCACCACGCCCGGCGAGACTTGGTTTTTCTGTATGGCCC
TAGCTGTCCTGGAACTTGCTTTGTAGACCAGGCTGGCCTCAGATCTGCCTGCCTTTGCTTCCCAAGTCCTGGAATTAAAGGTCTGCGCCACCAC
TGCTCTGCTAATGATGGATGAACATTCTCATAATGGCTGGACCACAAAGTTCTCCATTTGGTCTTCCCATTTATGTATGTATGTGTGTGTGTGT
ATTTGTTTTTGAGACAGGGTACCACCGTGTATCCCTGGCTGACCCTGAACTGGCTATCTAGACCAGCCTGGCCTCAAACTCATGGGAATCCTCT
TGCCTCTGCCTCCTGATTGCTGGGATGAAAGGTGTGAGCCCCTACTTCTGCCCCTCCCTTCTCAGTACTGATTTGTTTTTCACCAATGTAAAGC
ATGCAACGAACAGTGTCCAATAGGACCCGAGTATCTCTTTCTCGGGAGCAACTCTACATTTCTAGTTCTTTTCTGTCTAATAACTCTAAATTTA
TAAAATGTCTTCTTGAGTGTTTTGTTTGGTTTTAAGTATCTAACAACTCTTGCCATTGCGACTAAAGGTTTTCCTATCCTGCGTTTCCTGCTTG
CCTTGAACTGCATTTAACTTTCTTTCAGAACTCTTCGTCCACAGTTAATGGCTCAAGCTGTTTGTTTTAAGACTTACTGAGAATTCTGGCTTCT
TGGTTGAAGGGTTGAATTGATGCTAATGAATTTGTCTTACTAGGTCTATCGGGTCACAGTCTATCAGACAGGAGAAAGCGATACAGACTCTTTT
GAAGGAGATCCTGAGATTTCCTTAGCTCTAAGTATATAGATTCTTCAAAGAAATAAAACACAATGTTGAGATCAGAATTAGGACTATAGGTAGT
TGTGAGCCACCACCTGTGTGCTCAGAACCTGAGCCATCTCTAGCCTGTCACTATTTCATATAGGTCACCTTTTGTGTGTGTGTGTGTGTGTAGG
GGAAAGTACCTAGAGTCTACTCTATTAGCTAATTTCTGGTATATAGTATTAGATATATTCCTCATGTTCTTTGCCTATTTATGTAAAATATGAA
ATGCTGAAAATTAAGCTACATGGTATACTTTTACCTTAGCCACATTGAAGTTATTAGCCTTGCTATGAACACTGACTGAAGAGTTACTGTCTTT
TTTATTGAAGGACTATTGGAAGTGTACCTCATGCAATGAAATGAATCCTCCCCTTCCATCACACTGCAAAAGATGCTGGACCCTTCGTGAGAAC
TGGCTTCCAGACGATAAGGGGAAAGATAAAGTGGAAATCTCTGAAAAAGCCAAACTGGAAAACTCAGCTCAGGCAGAAGAAGGCTTGGATGTGC
CTGATGGCAAAAAGCTGACAGAGAATGATGCTAAAGAGCCATGTGCTGAGGAGGACAGCGACGAGAAGGCCGAACAGACGCCCCTGTCCCAGGA
GAGTGACGACTATTCCCAACCATCGACTTCCAGCAGCATTGTTTATAGCAGCCAAGAAAGCGTGAAAGAGTTGAAGGAGGAAACGCAGGACAAA
GACGAGAGTGTGGAATCTAGCTTCTCCCTGAATGCCATCGAACCATGTGTGATCTGCCAGGGGCGGCCTAAAAATGGCTGCATTGTTCACGGCA
AGACTGGACACCTCATGTCATGTTTCACGTGTGCAAAGAAGCTAAAAAAAAGAAACAAGCCCTGCCCAGTGTGCAGACAGCCAATCCAAATGAT
TGTGCTAACTTACTTCAACTAGCTGACCTGCTCACAAAAATAGAATTTTATATTTCTAACTATATGACCCCCAAATTAGACAACATGGGTATTA
TTTTTATACATTAAAGCCAGAAAACTGTCTTAGTCCACATAAAATTCACTTATAATTTATCCTGGAGAGTAAATATCGTGAATATTTTCTTCCT
TTTTAGGGAAATTTCACTTGTTTATTTTATATTTGTGTTTTAAGTAATTTGCATTGGCTGTTTATATTTTCCTTATATTTTAAATAATCTCCGC
TTGGAAGGACTTTGGAAGTGTATGTGAGAAGTCCTTTCCATCTCCTGCAGATGATGGTGGACCTTCCTCATCAAGGGCTACACAAGTACTTGAT
TTCTGTTTTTTTTGTTAATAATAAGAACATTTAATTTATTTAGTGTCTTTCATGTAAAGAGTTAAAGACTATGTGAAGGATTGTATATTTAAGT
TATTGAAATTCTGAAACTGTAGTAATCTAAAATGTGTGAGTTGTGGGCTGCAGAGAAGACTCAGCCAGTAAAGGCACCTGCTGTGTACACCCAC
TGACACACATTTGATCCTTGGAACCCCCAGGAAGAGTGAACCAGTTCCACAAAGTTGTTCCCTGATCTTCAAATCGATGCACGCACGCACGCAC
ACACACGCGCGCGTGCGCACACACACACACACACACACACACACACACACACACAGTTTTAAAGGCATGAATTGCATCTGGTGGTATGTAAGTG
AAAACACACGCCTTATTTTCCAGCATTTTCAGCTTTTTGTCATAGGGGTGTGGCACAAGTGTTGCAGTTTGTCCCAGGTTGAAAAGCCTGAGGC
TGGTAGAAGCGCCTTTTTGCCTCAGCTCCGTGGTTCCTCGTCCTTGCCTATGTTTCAGGCCTGTACTTAGGCTAGGTTTAGAAACCAGCCCATT
CAGAAAGACTGAATCAGAACATGGATAAAGTGAACTCATTCTAAGATGACTCGTCTATCCATGTAGATTAATCTCCTCCTTCATAATAGGCCTC
TTCCCTTTGATTGAAGGGTCACGTCTAAGTATAGAAAACATAAAACTGTAAGGTAGAGGAAGCCAACCATACCTTTGTATTAATGTTGCGTTAA
AGCTTCAGAGACAAGAACAAGAACACTCCTCCCACGTGACAGCATTTGAATAGGAGGCGGTGGGTGCGGCAGCCTGGGCAGCTTCAGTCCCGAT
TTACAATAAAGTACCTTGTGTGTTATTAGTTCTTAAATGTTTATTTAGAAATGCCATTGATGTTATTTATTTGCAAATAAATGGTTTATTGAAG
AATTGTCAAAGAGATTTGTCTTACACATTTTTTTGTCAGTGATTTTGTTTTTAAAGATTTGTTCATTATATGTGAGTACACTGTAGCTGTCTTC
AGACGCACCAGAAGAGGGCGTCAGATCTCATTACAGATGGTTGTGACCCACCATGTGGTGGCTGGGAGTTGAACTCACGACCTTTGGAAGAGCA
GTCAGTGCTCTTAACCACCGAGCCATCTCAGCAGCCCCCATCAGTGATCTTTTTATAATTCATTTTTATTTTATGTTCATTGGTGTTATGGCTA
TATGTCTGTGAGGGTGACAGAAACCCTGGAACTGGAGTTACAGACAGGTGTGAGCTGCCCTGTGGGTGCTGGGTAATTGATCCATTGCTCTCCA
CCTTTTTAAAATTTGTGTGTATGAATAATTCGTCCACATGTATATGTATTGTGTGCATACACTGCCTTGAACTACATGCATGCAGTGCCTGAAG
AGGCCGGAAGAGGGCATCAGATCCTTAGGACCCGAGTTCCAGGCTAGGAGCCTAACCCTGTCCTCTGTAAGAGCAGATGCTGCTCTTGACCACA
CAGCTGTCTACATGCCGCCTTTTTTTTTTTTTTTAAGGTTTATATATTTTATGTATATGAGTACACTGTAGCTGTACAGATGGTTGTGAGCCTT
CATATGGTTGTTGGGAATTGAATTTTAGGACCTCTGCTGGCTGCTTGCTCCGGCTCAAAGCTTTATTTATTATAAGTACACTGTAGCTGTCTTA
CTATAAGAGGGCATCAAGTCTCACTACAGGTGGTTGTGAGCCACCATGTAGTTGCTGGGATTTGAACTCACAATTTCTGGGAGAGCAGCCAGTG
TTCTTAACCACTCAGCTATCTCTCCAGCCCCTAATAGCTACAAATTTAAATACTTTAGGCAATTTTGCCTAGGGATTTCCCAATATATTATATT
GCAATGGATTTTCTTTTGAGTCCGAGAAATCTTTGCGATTTAACTTAAAGTTAAGATACTTGTGTGGAGGAGATGGCTCAGTGCTTGTCTTGTG
CCCTGATTCCATCCCCAGAAACCGTGGATATGGTGGCACTCATCTGTAACCACAGCATGAGAGATGACAGGCAGACCCACAGGCGAATCATCTG
GAAGCTTGCTGGTCAGCTGGCTTGCAGTATGCATCATGACACAAACAAGAGGCTACCACAAGGTGGAAGGCCGGAGACTGTTAGGTTTTGGGTG
TTGCTAAATACTGGCCCTGGTTGCCTCCAGGGGCTGGGAAATTCTCACTTAAACCCAGATGATGCTCTGTAATCTTCTCCCCCAAGTTATCCCC
GATTGGCGAATAAAGATGCCTGCAGCCTGTAGCTGTGTGGAAGAGAGACAGGTGGGGTTTGGGGGTCTGACGCAGGAAGAAGAAAGAGGTGGAG
AGAGGAGAGTGTAGGTGAGTCTGGAAGAAGTCAGTGCTCTTAATCCCTGAGCCATCTTTCTAGCCCCATATAGCAGTCTTTTCTAGTTTGTAGA
TTTTTGTTATGTTTGCAAACAAAACACTAGTGGAAAGAGACAACATTCACTATATGAATATTCTTTATTGATAATCAGATAATGTAAATAAAAT
AAGCTCAGAGGTAGTCATGGTCCCTCCCAAGCAAACGTTGTAATCTTAGATAATCTGAGAATTTATTTATTTTTCTGTGTAGCCCTGGCTGTCT
TGGAACTCACTGTGTACACCAGGCAGGCTGGATTTGAACTCAGAGTCCCCTCTGCCTCTGCCTCTGGAGTACTGGGATTAAAGGCGTGGTCCAC
CACTGCCACCACTCAACCTGGGCACCTTTAATAGCTTCTGCTTCTGTGCTTTTAATGGCAGGAATCACTTAGAAGCTTGTTAAACAAAAACCCG
CATGAAGTCTCTCGCACACAGCTGTGGGTTGGCTTGTGAGCTTAAGGTTTGATTAAACGGCCTGTGAGACACAGTACAGCATCTTATTAGTGAT
GCAAGTCTAGAACAAACTCGGTGAACAATCTGTTGTAGGCACCAAGAACCCCATGCTCTTCACCTAATCCAGCATCATTCTCTCCCGTACCTGA
GGTCACCAGGAAGGCAGAATTGAGCTCACAAACCTTCACTAGATGCCCCCTCCGAGACTGAGAATGAAACTCCTGTTGCAATAGGAAAATAAGT
GTGCTATATGGCTTTGTATATTTTTCCTAGAAACAGATACGTCTCAACCCAGCTTCAGCTCATACATTGGAAAGCAGATCTGATGTTAATACCA
TTTTAGTACAGGCAGAGTCTGAGAAAACTGGGACTGTTTTTGCTTGTTTGAAACGGGGGTTTTTCTGCGTAGCCCTGGCGGTCCCAGAACTCTG
TAGAAAAGACTCGCCTTGAACTCACAGAGATCCACCTGCCTCTGCTGCCTGGGTGCTAGGATTTTACCATCTCAATTCAAAAATCCGCACGCAT
GTAAACTAGGCACTTTGTGAAAGTTTCCTTGAAAACGACGTGGTTTTTGGTAACCAGCATTTCCGTTTTCAGGTCACCTTTAGTAAGGTATATT
GCTTTCAAAACAGTTACAGACCCAAGTATTCCTTCTGCTTTCTCTACCTGGCAGAGGGCAACAGAATAATTTCCTGGTTATGTTTTGTCTTGGG
GGTAGTTAATGGAGATTAGACCCAAGCCTTTGCATATGCTAAGTACACTGTCACTGAGCTACACCCCTCACCCTGTTTTAGTGATTTTTACACT
GGGTTTTGATATATATTTCTGATTTAAGTAATGTAACCTGAATATGGACACAGCGGACCAGGTCTGTAATAGCTACTCCAGCGTCTACGGCAGG
CAGATCCAAAGCCAGTTTGGCTTACTATGAGTTCCAAGTGCAGCCTACACAGCTTAGTGAAATGCTGTGTCTGAACAAAAAGTAACAAAATAAA
GACGTAGCTGTGGCTCAATGGCCCTGTGCAAAGCCCTGGCACAGTCTCTGGTAAGACACGGGCACTTGCAAGTTGTTTCAGTACCAAGTCTCCG
GTTAGGCCCATGAGCAGCTCAGCGAACACCTCATTTCCTTTCACGGAATCTTAGGAAAGGGTCAACCAGTCAGAACTCAGAACACCAGCTGATG
GCGAACCAAGTTTGCTCTTACATATCTTACTGGTTTTGCTTTTCTAAGATGGCTAGTCCAGGCTAGCCTTGAGCTGTGTCGATCTTGCCTCAGC
CTCCCAAGCGCTGGGATAATAGTTCACCACCACACCTGGCTTCGTCTTATATATATACTTCAAATGTAATATACCCAGATCCCTTTTTGAACTA
TTAATACGCGCCTTCTGGGATATAAGTTAATGCTTATTTAAATAAAATGATCAAGATACTCATGCTATTTCCAAATTTAGAAGCGCTTAAAAAG
CTATGCAGACTATTGATATTAAAATATAGATTAGGTTTGGCACCAAGAAAGGGCACCACGGTGCCCACGTATCTCTTTAGAGTCTCAAGCCTTC
ATCGCCTGTCAAACCCCGCATGTTCTTACTTCCTCTGGGAAATGCATGTCACTTAGAAACCTGCACCTCGGAGTTACTGTTAATGTTCCTGTTT
GATATTTATCCTTTTTTTTTTCTTTTAATTTATTTACTTTTGAGACAGGGTCTGTCTGTGTAAACGAAGACATCCGCCCACCTCTGCCTTCTGA
GTGTTGGGATTAAAGGTGTGCGCCAACACGCCCAGCTTTCTCTTCTTCTAATGTTAGAATAAAGTTACTTTTACAGGGGACACTTTACTTCGAA
CTGTAAGAATTTGTTTTCTGGTCTAGAAGGCAGATTGGCTAACAGCTAATGCCTTTCCCTCAAGCCTCACAGCTCACCCATTTGCATTTATCTT
TGGATTTTGTTCTTTGACATACACGTGAAAAGTGTGTATGTGTGTGTGTGTGTACTAGTAAGCTGAATGTTGCCAAACATAAAACGCATTTACA
CAAAATTACTCAGATCCTTAGCGTTCTGGAAAGGTCTGTAGCCGTAGCTGAGACACTTCCCCATCCATTTATTTCCTGTTTTTTGGCTAGACTC
TGAGAAATTATTTCTCAAGCAGATCCTTGTCTTAAAGAGCTCATGGCGTGCTAGAGAAGGCCAGCACAGAGCAGTTCCACACCACGATGCTGTA
AAGTCACTAGTCAAAGGAACTTCTTTGAAGGATCGGCCTTGAACTTGACCACCTGGAACTCCATGGGCTTCCCCGTTCAACACGATGCTTGGAA
TTCTTCCTTTTAAAAAATTTATTTATTTTACGTGTATGACTACATGGTTGCTGTCTTCAGACACACCAGAACGCAGCCTCGGATCCCATTACAG
ATGGCTGTGAGCCACCATGTGGTGGCTGGGAATTGACCTCAGGACCTCTGGAAGAGCAGTCAGTGCTCTTAACCCCCCATGGAATGGAGTTTTA
CTGACCAGGAAACAAGACGTGACACAATCAGAATCCAGACCTCGCACCGACAGCACTTACTGTGTTGTGTTTAGGGAGCAGGAGAGCTTTGGGC
AGCCCAGCTCCCCAGGTTCTGTGTGGAAGACGGACTTGGCGAAAATACCCATGACCAGCAGCTGGGTCAGCGGCGGAGGGACGCTAACTGGCCT
GGAGAGGCTGTATACAGCAGCTTCCAGTGACTGGATATGGGAATCGAACAGCCTTGCATGGCGTCTCAACAGTCAACCGGATGAACTGCAGTTC
CTAGGCATCGTAGTCAGGTGGGTGTGTCTGCTGAGCAGCTGGAGTTGCATCTGAGGCTCACGCAAGCGACTGGGGTTATACAGGAGGGGTCATC
TTCCTACGACTGTGGCATCAGGTGCAGGGGGCAAGCGCAAACGCACGCGAGACAGAGGCCACTGCATCTGTACCCACATCAGACATGTTTGTCC
ATTTTCTCTCATTTGGCCTTAGACCATTGGCAAGAGTAAATGCTCTTAGTCCCGTTATCTAGAAATTTCTTCCTTTGCGGAGAACCACTTATAG
ACAATATCAGCTCTCTACAAATAACACGAAAGGTCGTAACACAGCAAGTGACCAGAAAGTGCCCGTCCTTGCGGCTCTGATCCACGTGGCTCTC
CGTAGACAAATTGTTTTTCTTGTAGGGATATCTGTTTTGCTTCTGAACTTTCTTACAAGTGTTTGGGACTCCTTCGGGGTGGCGTTTGTGTGGG
CAGCATGAGCTAACCAGTGACCTCCACTTTATCCAGACTGAAGATGGATTTGGTCGTTACAGGTCGCTCTAGGCCAGGTTTGGAAACATCCGGT
TTTCTTCTTAAGGAAAACAGTGGAGTCTGTCCCAATCGCAGTCCCACAGGAGGGAGGGTTGCCATAACCGGGAGTAACCAATCCCTGATTCCCG
GAAGTCGAGTTTCACACCTTGCTTTATTTAAAAAATACGTACAAAAGAGTCATGAAAAACACCCCCAGACTAGGCTTTGTGGCAGCCGAGTGGC
TTGGCATGAATTTGTACAGCGGAATCATCGGGCACGAAGGATTGGATACGGAGATGGAATCCGGCTGCCATCGCAGCGGGAGGTGAAAATCCTT
TTTAAGAGCACTGAAGGGCTGGGATTTCCCTGGAATAGTAAGCTTCGTGAGGTAGGAGTCGTGTCCAGGGACTGTAACCTGAGAAGGAACAAGA
AACAATTACTTGAGTGATTTCAGATGGGTGTGAGGTCCACGCAGAAGACAGATCTCCACCCCTTTGAGCGGTGCATGCGTGGCTTGAAAGCGGT
CCACGCCTCCGGGGCTATACCCCCCGGAAAGTTGCCAGTGTTTCTTGATGTAGGCCTTTGGTGCAAATGCTACCAAGAATTACAAAATGAAGTA
AGAACAGAGAGGGGGTTCAGCATCTAGTGAAGGAAAAGACTAAATGGAGGAAGCCACGACTGATTCTGCCACGGTCATTTTCAGTGTGCCGGGG
TAAAGGTGCTTTTTTTGGACCAAGCCTAGAGTATCCTTATTGGATATCAAGATTGTGAGTAGATTCTTCCTTCAACTTACTCCCATTCTCCTTG
CCACTGACCTAAGGTGTATAACTCTACTTCACTCCCCACCCCTCCTTTTTAAAAAGAAAGAAAGAAACAAAACAAAACCAAACCCAAAACCTTG
CACAAGAGCGGTTTTTTTGCCTGCACGTGTCTACCACTTGCATGCAGTCCTTACAGAGGCCAGAAGAGGGCAACAGATTTCCTAGAACTGGAGT
CGCGGATGGTCAGTTGTGAGCCACCCGGTGGATTCCAGAACTTCAACCCAGACCTTCGGAGCCCTTCTACCACTTTCTTTTAAAACTTTTAATT
GTGCCAGGCGGTGGTGGCGCATGCCTTTAAGCCCAGCACTTGGGAGGCTGAGACAGGCGGATTTCTGAGATCGAGGCCAGCCTGGCCTACAAAG
TGAGTTCCAGGACACCCAGGACTATACAGAGAAACCCTGTTTTGAAAAAACAAACAAACAAACAAAACCCCCAAAACCAAAACCAAAACCAAAC
AACTTTCAATTGTTTTAAAAACTAGTTTTGTTCTTGTGAATCTTCAGGTAGGTTCCGATTGCTCTTGGAGTTGCTACATAGACCAGGTTGACCT
CAAACTCACTGGGTCCACCTGCCTCTGGGCAGTGCTGGCATTCAGGGCTTCCTTTTGAGCACTTTATATATTCTAGAAACTCATCTCTTAATGG
AGAAGTCATTTGCAGATCTTTTTCCCCAGGTCTGAAGACACACGGACAATCTGCTCTTGGCTCCTTTGCCATGCTTCCTGGTTCGTTTTTCTTT
GTCTATCTTAATTTTGTGCCTTTGGGGCTCTATCTAAAAAAAACCCATCGCTTTACATAAATGGCTTGAAGTGTTTCCATTGTTTTCAAGCACG
CACACACACACACACACACACACACACACACACACACACACACACAATTTATACCTAGGAGAAGCTCAAACATCTGCATCTGCAGCTGTGCCCA
GGAGAAGTGAATGGTAGGTCTGGAGCCACCCGGTAGCTCTGTTCCTGGAGCTGGGGGACCCAGCTGGCACCACCTGTCTAAGTTCCTGATTGAC
AGGTAGGACTGAGGGGCTGTCTTGCTACAGTGCCAATGCTGTGGTTGGCAGGGACTCCCCTAGATCTCGTGGGGCCAGACTGAGACAGCACCGG
GGCTTGTGGCATGCCACATGGTAGCAGGGGGGGGGGGCTGTCTTTTCTTCAGTAAAATGTGCTCTGGACCAAGGCTTCAGCCAGAGTCCAAGAT
GGCTGACAGCTCCTATGCCAAGATGGTGTTTGGCAGTGGCTATGGAAGGGCAAGGAAAATGACAAAGAAAAGCCACTTTCAGAAGAGCTCTCTA
GATTGGATTTAAACCTTGGGATTCCTCATTCATGGTTCCCTGCCACACTGGTGCTTGCTGGCATCTCCCATCGTCCTCAGTTCACAGGCAGGCC
TGGGTGCCAGCGCTCAAGCTGCTGAGTGTTTCTCTCTTTCCTCTCCGGTCATTTCCAGGCACACCTTTGTGCATCTTCCTAATTCCCCCTCTTT
CGCTCCTACCATGTGGTGTCTGCTTAGCTGCCCTGACTTCTCTGGCAGGGCTACCTGTAAGGCACCTTGGCTTACACTGGCTTCCTCCTGGGCT
GATTGATTAGGTTATCCGTGTGTCTGTGTGTGTGTGTGTGTGTGTGTGTGTGTGTGTCTGTGTACGCGTTAGAATAGCCGTCTTGAGCTGGGGA
TGGTTCACTCGGTGAAAGCGCTTGCCCAGCAACCCCGACATGATTCAGTCTCTAGAATGTATGCTGGAAGGAGCAAACTGCCCTCAAAGCTGCT
TACCAACTTCTCCAAGTGTGCTATAGCCCACAGATGCGTACATAATTGAGAAAACATGGTAGGTGCTTGGTTAGGAAAACATATGACTTTCCTC
CCGGAGACAGTGTTTCTCTGTATAGCCCTGGCTGTCCTGGAACTAACTCTGTAGACCAGGCTGGCCTTGAACTCAGATCTGCCTCCCTCCGCCT
CCCGAGTGCTGGGGTCAAAGGCACATGCCACTGAGATGCCAAAGCTTTGAAATTCAGACTCCATTTTAGAAAACCTGACCTAAGTGTGAACTCA
GGTAGACCAACAGGCTGGTATCTAATAGCATTAGTTTCCAAGTTGCCCCCCAACAGAACCTGAGCCTAGCTCCCGTCTCTAGTCTAATCCCCAA
CAAGAGCAGAGTTTCCAGGTTACACCCTCAACAGGACCAGTGTCTCCAGGCTATTTCCCCAACAAATCTGCACCCCCAAGTTACAAGCCTACCC
TCTGCCTAACGACCACCAATGCAGAGGGGAGCAGAAGTTAAGTCTATGATAGACTCCCAGCACCAGCCAATCATGTTAAAGGCCACAGGAGCTT
TCCAATGAGATGCTTGCACATGTACTCCCTGCTTGCTGCTTACTATAAAGCCTTGACCCATAGACATTCTGGGCTCCCCACCACCACCACCAAA
ACTGTCCTGCCCTGCGTAATGGTGGTGCGTTGGGGGATGGGCGGGAGGGTTGTTCGAACAGAATAAAGACCCTGGTGAGTTGCACCAGATTGGC
TCCTGTGTGTGTTTTGGGGGATCACTAACATTTCCCTGGCGCAACACCATCACTGCGAGACTATATGTGATTCCCCCTCATTGGTTTGTTTCTG
CCTTCCACTCCTCTCCTATTGCAGGCCTCATGTTAACCTTGCCTTCCAAGCCTTGCTG
MOUSE SEQUENCE—mRNA
GAGGAGCCGCCGCCTTCTCGTCGCTCGAGCTCTGGACGACCATGGTCGCTCAGGCCCCGTCCGCGGGGCCTCCGCGCTCCCCGTGAAGGGTCGG
AAGATGCGCGGGAAGTAGCAGCCGTCTGCTGGGCGAGCGGGAGACCGACCCGACACCCCTGGGGGACCCTCTCGGATCACCGCGCTTCTCCTGC
GGCCTCCAGGCCAATGTGCAATACCAACATGTCTGTGTCTACCGAGGGTGCTGCAAGCACCTCACAGATTCCAGCTTCGGAACAAGACACTCTG
GTTAGACCAAAACCATTGCTTTTGAAGTTGTTAAAGTCCGTTGGAGCGCAAAACGACACTTACACTATGAAAGAGATTATATTTTATATTGGCC
AGTATATTATGACTAAGAGGTTATATGACGAGAAGCAGCAGCACATTGTGTATTGTTCAAATGATCTCCTAGGAGATGTGTTTGGAGTCCCGAG
TTTCTCTGTGAAGGAGCACAGGAAAATATATGCAATGATCTACAGAAATTTAGTGGCTGTAAGTCAGCAAGACTCTGGCACATCGCTGAGTGAG
AGCAGACGTCAGCCTGAAGGTGGGAGTGATCTGAAGGATCCTTTGCAAGCGCCACCAGAAGAGAAACCTTCATCTTCTGATTTAATTTCTAGAC
TGTCTACCTCATCTAGAAGGAGATCCATTAGTGAGACAGAAGAGAACACAGATGAGCTACCTGGGGAGCGGCACCGGAAGCGCCGCAGGTCCCT
GTCCTTTGATCCGAGCCTGGGTCTGTGTGAGCTGAGGGAGATGTGCAGCGGCGGCACGAGCAGCAGTAGCACCAGCAGCAGCGAGTCCACAGAG
ACGCCCTCGCATCAGGATCTTGACGATGGCGTAAGTGAGCATTCTGGTGATTGCCTGGATCAGGATTCAGTTTCTGATCAGTTTAGCGTGGAAT
TTGAAGTTGAGTCTCTGGACTCGGAAGATTACAGCCTGAGTGACGAAGGGCACGAGCTCTCAGATGAGGATGATGAGGTCTATCGGGTCACAGT
CTATCAGACAGGAGAAAGCGATACAGACTCTTTTGAAGGAGATCCTGAGATTTCCTTAGCTGACTATTGGAAGTGTACCTCATGCAATGAAATG
AATCCTCCCCTTCCATCACACTGCAAAAGATGCTGGACCCTTCGTGAGAACTGGCTTCCAGACGATAAGGGGAAAGATAAAGTGGAAATCTCTG
AAAAAGCCAAACTGGAAAACTCAGCTCAGGCAGAAGAAGGCTTGGATGTGCCTGATGGCAAAAAGCTGACAGAGAATGATGCTAAAGAGCCATG
TGCTGAGGAGGACAGCGAGGAGAAGGCCGAACAGACGCCCCTGTCCCAGGAGAGTGACGACTATTCCCAACCATCGACTTCCAGCAGCATTGTT
TATAGCAGCCAAGAAAGCGTGAAAGAGTTGAAGGAGGAAACGCAGCACAAAGACGAGAGTGTGGAATCTAGCTTCTCCCTGAATGCCATCGAAC
CATGTGTGATCTGCCAGGGGCGGCCTAAAAATGGCTGCATTGTTCACGGCAAGACTGGACACCTCATGTCATGTTTCACGTGTGCAAAGAAGCT
AAAAAAAAGAAACAAGCCCTGCCCAGTGTGCAGACAGCCAATCCAAATGATTGTGCTAAGTTACTTCAACTAGCTGACCTGCTCACAAAAATAG
AATTTTATATTTCTAACT
MOUSE SEQUENCE—CODING
ATGTGCAATACCAACATGTCTGTGTCTACCGAGGGTGCTGCAAGCACCTCACAGATTCCAGCTTCGGAACAAGAGACTCTGGTTAGACCAAAAC
CATTGCTTTTGAAGTTGTTAAAGTCCGTTGGAGCGCAAAACGACACTTACACTATGAAAGAGATTATATTTTATATTGGCCAGTATATTATGAC
TAAGAGGTTATATGACGAGAAGCAGCAGCACATTGTGTATTGTTCAAATGATCTCCTAGGAGATGTGTTTGGAGTCCCGAGTTTCTCTGTGAAG
GAGCACAGGAAAATATATGCAATGATCTACAGAAATTTAGTGGCTGTAAGTCAGCAAGACTCTGGCACATCGCTGAGTGAGAGCAGACGTCAGC
CTGAAGGTGGGAGTGATCTGAAGGATCCTTTGCAAGCGCCACCAGAAGAGAAACCTTCATCTTCTGATTTAATTTCTAGACTGTCTACCTCATC
TAGAAGGAGATCCATTAGTGAGACAGAAGAGAACACAGATGAGCTACCTGGGGAGCGGCACCGGAAGCGCCGCAGGTCCCTGTCCTTTGATCCG
AGCCTGGGTCTGTGTGAGCTGAGGGAGATGTGCAGCGGCCGCACGAGCAGCAGTAGCAGCAGCAGCAGCGAGTCCACAGAGACGCCCTCGCATC
AGGATCTTGACGATGGCGTAAGTGAGCATTCTGGTGATTGCCTGGATCAGGATTCAGTTTCTGATCAGTTTAGCGTGGAATTTGAAGTTGAGTC
TCTGGACTCGGAAGATTACAGCCTGAGTGACGAAGGGCACGAGCTCTCAGATGAGGATGATGAGGTCTATCGGGTCACAGTCTATCAGACAGGA
GAAAGCGATACAGACTCTTTTGAAGGAGATCCTGAGATTTCCTTAGCTGACTATTGGAAGTGTACCTCATGCAATGAAATGAATCCTCCCCTTC
CATCACACTGCAAAAGATGCTGGACCCTTCGTGAGAACTGGCTTCCAGACGATAAGGGGAAAGATAAAGTGGAAATCTCTGAAAAAGCCAAACT
GGAAAACTCAGCTCAGGCAGAAGAAGGCTTGGATGTGCCTGATGGCAAAAAGCTGACAGAGAATGATGCTAAAGAGCCATGTGCTGAGGAGGAC
AGCGAGGAGAAGGCCGAACAGACGCCCCTGTCCCAGGAGAGTGACGACTATTCCCAACCATCGACTTCCAGCAGCATTGTTTATAGCAGCCAAG
AAAGCGTGAAAGAGTTGAAGGAGGAAACGCAGCACAAAGACGAGAGTGTGGAATCTAGCTTCTCCCTGAATGCCATCGAACCATGTGTGATCTG
CCAGGGGCGGCCTAAAAATGGCTGCATTGTTCACGGCAAGACTGGACACCTCATGTCATGTTTCACGTGTGCAAAGAAGCTAAAAAAAAGAAAC
AAGCCCTGCCCAGTGTGCAGACAGCCAATCCAAATGATTGTGCTAAGTTACTTCAACTAG
HUMAN SEQUENCE—GENOMIC
CGAGTAGCTGGGATTACAGGCACCTGCCACCACGCCTGGCTAATTTTTGTATTTCTAGTAGAAACGGGGTTTCACCATGTTGGCCAGGCTGGTC
TCGAACTCCTGACCTCAGATGATCCGCCCGCCTCGGCCTCTCAAAGTGCTGGGATTACTGGCGTGAGCCACCGCACTTGGCAGTTCTTTTTTTT
CTTTTTTCTTTGAGATGGAGTCTCGCTGTGTCGCCGAGGCTGGAGTGCAGTGGCGCGATCTGGGCTCACTGCAAGCTCCGCCTCCTGGGTTCAT
GCCATTCTCCTGCCTCAGCCTCCAGAGTAGCTGGGACTACAGGCGCCCACTACCACACCTGGCTAATTTTTTTGTATTTTTTTAGTAGAGACGG
AGTTTCACCATGTTAGCCAGGATGGTCTCGATCTCCTGACCTCGTGATCCACCCGCCTCAGCCTCCCAAAGTGCTGGGATGACAGGTATGAGCC
ACCACGCCCGGCCAGTTCTTTTAGTTTTTAAAGCAGTCTCATTTCAGTTATTAATATTGACTTCTTCATTTTGAAACTTCAAATAATATTTGAT
TCAAAGCTTTCCTCCTCTTACCAGAGCAGGCTGGACCTGTGGCCCTGGTGACCTGGAATTGGAAATGGGGGTGTGGGATGTAGGGCATAGGTCT
TCTTGTCACATCCCATTCTGTCTTCCCAATTATTACTTGTGGTACTCAGAATTCCAAGAATGACTCCCAGTGACCTTGCCTATGGATGATCCCC
TCCCCTTTAAGCGTGGGAGGAACCTGTGAATATGAGGAGATATTACTCCCATCATTCTGTTACATTACATGGCAAAAGGGAGATGATCTGGGTT
GGCCTCATCAAATCACATGAGCCTTTGAAGTCAGAGTTTTTTTCTGGCTGATAGCAGGAGAGTAAGTCAGGTTCAAAGCACAAGGGTGATTCAA
CACCCTGTTGCTGGCTTGAAGATGGAGGGGAGTGTGTAGCAAGGAATGCAGGAAGCCTTCAGGAGCTGAGAGTGGTCCTCACTGCTAGCAAGAA
AAAGGGGACTACCGTCCTACAACCACAAGGAACTGAATTTGGCTGACAATTTGAATAAGCTAGAAAGCAGGTTCTTCCCTAGATCCTTAAGAGC
CCAAGCTGGCCCACACCTTGATTTAGGCTTTATGACACCTGGAGCAGAGGAACCAGGTGAGCCTACCTGGATTTCTGATATATGGACTGTGAGC
TAATAATTTGTGTTGCTTTAAGCTACTACATTTATGGTAATTTGTTACACAGCCATAGAAAACCACACATGACTCCTGCCATCCCGATACCAGT
TGGGGTGGCAAGGAGGAAGGAGAGGAAAATTACATTTCTTTACATAATTGCCCTTCAAAATTAGTTGCCCTTTTATTAGCAGGACATAACTGTG
GTTCATTGTATGAACAACCTCAACTAGATCATGGGAAACATTTCTGTCCCTTTCTGTTGGTTGTCCCAGTTTCTCTGATTATTACCAATTGACC
ACCAGTTCTCTTCATCTGGCAGGTGTCCAAATGATATTTCTCCAGAAAGACTCGTGGCATGGTCCTGGTAGGAAGTTGAGTAGGGGTATATGAA
TGAAGCTCAACTTCTTTCTCCCTAGTCCAGCACATTCTACCAATAGATATTCTATGTCTTTTGCTTCCAGGGTTCTATTACCCAGGTAGTGGCC
ATTTTAGGAGTTACCAGCAGAACTGCTTATACTCCTTCCATCTTCTCCAGGAACCACTTAATCTGGGGGAAACTCCAGCAACCTTTCCACCCCA
AAGAGTGGGGATGCTGTTCAACTTCACTACCTTTCTTCTGCCAGCTGAATGGCTCTGTAACTTTCAGACTAGATACGATCACAGATCTCAGATT
TAGGTCAGCTGGGAGATTATAAACTCTTCGTTCCACGAAAAGATGACCTCTATTATTCCTGGTCCTTCCTTAAAACTCTTAAACGTGGCCGGGT
GCAGTGGCGCACGCCTATAATCCCAGCACTTTGGGAGGCTGAGGCGGGTGGATCACCTGAGGTCAGGAGTTCACGACCAGCCTGACTAACATGA
TGAAACATGTCTCTACTAAATACAAAAAATTAGCTGGGTGTGGTGGCACATGCCTGTAATCTGAGCTACTTCGGAGGCTGAGACAGGAGAATCA
CTTGTATCTGGGAGGCGGAGGTTGCACTGAGCCGAGATCAAGCCATTGCACTCCAGCCTGGGCAATAAGAGCAAAACTCCGTCTCAAAAAGAAA
AAAAAAACTCTCAATGGTAAAAGTTATCAAACCAGTTCAGCCTGTATTGCCCTTGTGGGAACCAGAGTCTCAGGCCAAGAATTGGGCCTTATTT
GAACTTAGCTTTAGAGAATTGAGCGTTCTTAACAGGGCAATATTCCTACTAAAATCTCTGAAATATTTGTTATCTTCTAAGAGCAGGAACTCAA
TTATGCTCCCATATCATTGTGTAATATTTTTCCTCTTAAAAATTCAGTTCATAAATGTCCAAAAAAAGAGATCTATTGTAACTAAGTAGAATTT
TATCTGAGTAAAACAAGGAAGGTCAATATTGGAAATCTATCAATATAATCTCACCACATAGACAGATTAAAGGAGAAAAGCCACAGGGTCATTT
CTAGGTGCATAAAAAAGCAGTGTATAAAACTGAACACTGATTCATACTTTTTTATAAGCTCAAAAAACCAGGAATAGAAGGAAACTTTTATGTA
ATAAAGAGCATCTACCTAAAATTTACAGCAAACATCACATTTTAAAAGCATTTTTACTAATGTCATGAATAAGATGAGGATGCCCACTATCATT
GCTACTATTCCATTCCAATGGTTGTAAGAAAATGAAAAAGAGGTTTAAGGAAGAGACAAGTTAGTGATTTTTGCATATGATATTATTAACATAG
AAAATCCAAGAGCATCTACTGAAAAACTAATAGTACTGTATAACAAAACAAAATACAGCCTGTAATCCCAGCACTTTGGGAGGCCAAGGTGGGT
GGATCACGAGGTCAGGAGTTCGAGACCAGCCTGGCCAAGATGGCGAAACCCCATCTCTAGTAAAAATATAAAAATTAGCCAGGTGTGGTGGCAC
GCACCTGTAGTGCCAGATACTCAGGAGGCTAAGGCAGAAGAATCCTTTGAACCAGGGAGGCAGACACTGCAGTGAGCCGAGATTGTGCCACTGC
ACTCCCGTCTGGGTGACAGAGTGAGACTCCGTCTCAAAAAAAACCCAAAAAACAAAAAACAAAAACAGAAAGAACAAAAAAGCAGTATTGCTGG
CTGGCTATAAGATCAAAACACAAAAATGAGTAATTGTTTCATATGTTAGCAATAACCAAATAGAAAATAAAATGTTACGGCTGGGCATGGTGGC
TCACGCCTGTAATCCCAGCACTTTGGGAGGCCGAGGTGGGCGGATCACGAGGTCAGAAGATCGAGACCATCCTGGCTAACACGGTGAAACCTTG
TCTCTACTAAAAATACAAAAAAAAAATAGCCGGGCGTGGTGGCAGGCGCCTGTAGTCCCAGCTACTCCGGAGGCTGAGGCAGGAGAATGGCATG
AACCCGGGAGGCGGAGCTTACAGTGAGCCAACATCGCACCACTGCACTCCAGCCTGGCTGACAGAGCGAAACTCCACCTCAAAAAAAAAGAAAA
GAAAATTTTAAAAGGTGTAATTTAAAAACAGCAATAAAACTTATAAAGCACTTCAGAAAAAAGCCAGCCAAACATACAACAGCTTTAAGGAGGA
AACAATAAAGCATTATTAAAGGATGTTAAGACAGACCTAAAAATGGAGTGATATATCATGTTCTTATCTGGGAAGAATTCATACTGAAAATAAG
TCAAATTAGTCTAACAATCCAATGCAATGAAAAGCAAAATCCCAAGAGGATTCTTTTTGTGAATTAGACAAACTGGTTCTAAAAGTTAACATCC
AGGAAGAGCCAAAACAATACTGAAAAAGATAAAGAGAGCTTTTCCTGTTACATATTGAAACTTACTCTAAGGTTTACTATTCTGTTGCCCAGCA
AGTCCTCTTATAGGTATGCAGCCTACTGTCCGTCTTGTTTATGGATTCATAAATTTACAGTGAATGTATAAAACACAGACTTAGTGGACAGGCA
TCAAATTGATCATTATCAAGAGAACGGAGGGGAATGGGATAAAGAATAACAGTGAAATTTTAATTTTTTAAGCAATGTTTCATTAAAAAAATTT
ATTTAGGCCAGGCTCAGTGGCTCACACCTGTAATCCTAGCATTTTGGGAGGCCAAGGCAGGTGGATTGCTTGAGGTCGGGAGTTTGAGACCAGC
CTGGCCAACATGGTAAAACCCCATCTCTACTAAAAATACTAAAGATTAGCCAGGCATAGTGGTACAATCTTGGCTCACTGCACTCAGTGTGGGC
GACAGAGCAAGACTCTGTTTCAAAAATAAATAAATAAATAATAAAAAAAATATGTTTGATGCAGGAATGACAAATGTGAATTCTGGGTAGTGGT
TTCTTTTTTTCTTTTTTTGTTCTTCAGAGCAGAAGAACTTGAGGCGGTGCTTTCTTTTGTTGAATATTAATTGTTCTGTGAATGTTTCTGTATG
TTTCAGTCAAACAAAAACTTGATTAATTTTCAAAGTAAAAACAAAAAGTCTTTGTATTCACTACTTGCTGGGATTTTGCTTTTTATTTTTTACT
TTATTTATTTATTTATTTTTTGAGATGGAGTCTCACTCTCACTCTGTCTCCCAGGCTGGAGCGCACTGGCATGATCTCTGCCTCCCAGGTTTAA
GCAATTCTCCTGCCTCAGCCTCTGGAGTAGCTGGGATTACAGGCGTGCACCACCATACCCGGCTAATTTTTTGTATTTTAGTAGAGACAGGGTT
TCACCATGCTGGCCCAGGCTGGTCTCGAACTCCTGGCCTCAGGTGATCCACTCGCCTCACCCTGCCAAAGTGGTGGGATTACAGGCATGAGCCA
CCATGCCCGGCCTATTTTTTACTTTTTTATTTTCCAGGACAGAGTCTTGCTCTGCCGCCCAGGCTCGAGTGCAGTGGCGCGATCTCGGCTCACT
GCAACATCTGCCTCCCAGGTTCAAGAGATTCTGCTGCCTCAGCCTCCCGAGCAGCTGAGATTACAGGCTTGCACCACCATGCCCGCCTAATTTT
TGTATTTTTAGCAGGGACGGGGTTTTACATGCTCGTCTCAAACTCCTGACCTTCAGTGATCTGCCTGTCTTGGCCTCCCAAAGTACTGGGATTA
CAGGCGTGAGCCACTGCGTCCGGCCCGGATTTTGCCTTTTAAATAACACTATTCAGTTCCAGATGTTTGGAAAGACATAAGGAATTTGAACTAC
TTCTGTAATTTTAGATGGTCTCATAAATATTGTGAAACTCAGCATTCTAAACACGCTTTCTGAGCTAAATCTTGTTTAGTTTTCACCATGCTTT
AAAGATTAGTTGTGGCCAGGCACAGTGGCTCACGCCTGTAATCCCAGCATTTTGGGAGGGTGAGACGGGTGGATCACCTGAGGCCAGGAGTTCG
AGACCAGCCTGGCCAACATAGTGAAATCCCGTCTCTACTAAAAATACAAAAATTAGCCGGGTATGGTGGCACAAGCTTGTAATCCCAGCTACTC
GGGAGGCTGAGGCAGGAGAATCACTTGAACCCAGGAGGTGGAGGCTGCAATCAGCTGAGATCGTGCCACTGCACTCCAACCTGGGTGACAGAGT
GAGACTCCGTCTCAAAAAAAAAAAAAAAAGAAAGACAAAAGATTAGTTATGACTAATAAATACAAGTACGAAGTGGCCCTTGGGTCAAGATAAG
TCTGTTGTAGACTTTTTTTTTTCCCCTTGACACATGGTCTCACTCTGTCACATACGCTGGAGTGCAGTGGCAGGATCACACCACAACCTATCCG
GGCTCAAGCCATCCTCCCAGCTCTGGCTCCCAAGTAGCTACGACTATAGGTGAGTGCCACCACGCCTGGCTAATTTTTTTTTGCAATTTATAGA
GACAGGGTTTCGCCATGTTGCTCAGGCTGGTCTCAAACTCTTGGCCTCAATAGATCCTCCAGCCTTGGCCTCCCACAGTGCTGGGATTACAGGT
GTGAGCCTTGTTTGCAGAGTTTAGGTCCCAGAGAATATAAAGACACTCAAAGTATCTCAACCTCTTGCAACACCTCTTGGGCATCAACAACAAC
GCTACTAGAAATAAGCAGTTTGCCTCTAGAGAAACTTCAAGTAAGCACAACAAAGGGCAAAGTGACACAAGATAACTGTGGAATGCAAGCAAAC
AGCAGCAGCTGGTCTGTATTTTTTTAAATGGCAAAGTCACAAACAACAGCAACTGCTTTTAGAGTGAGCAGGTAGTTTGCTTTCTTTTTTTTCA
AATGCAACTCACTTTTTAAGTGATGACATGCACTTGCGGGTGGGTAGGTGTGAGTTCATTTGTGTTTTGAAATCCCAAATTAATGAGCATGATA
AACCGCTATTGCTGGCCCTGTCTGACTCTCTACTCTGAGTAATTGGTGGGTTCCCCCCAACCTTCTCCTAACCCTCACCCTTTTTCCCTTTCCA
TTGACTTGGGACAGTCCTTGTCAAAGTGCCAATCACAGTGGGAAGGAGGAGAAGGGGGGAGGTCACAGTGCATGGGATTCAAGGTCACAGTGGG
CGGAGCAACGGGGATCACCACGCAAGTAAGTCAAGTTCCCGCTGCTCAAGTCCAGCTGTCTCCCACGGTAGTGCAACCCTTGGCGGACGACCCA
GGACGCGTTGACGCGGCAGTCGCGCCATCTCTTCTTGCCTGCATTTATCTGCGCTGTTTGAAGCCGTCTGACCCATCAGGACCCAGAGGCTGTC
TGATCACATTATTGGGCTGTCTGCTGTCTTCGGCTTAGGAGATCCTGGTTGGCCTGAAGGGAAGAAGATCAGAGGAAGACCTATCGAGGATGGG
TTTCTCCTGCTTCTCCTCGGGTTGGCGCTGCCCAGGATCCGCTTCCTGGCCTCGGTGTACTCGGCTTCCCACTGTGCCAGGGACTTGACTGGAA
GGGCGGGCCTGCTAGCGGACTTGGGGCTGCTGACCACACCGTTGCTGGTGGGCCTCTTGAGGATGCGGACCTGTGGAGGGGGTCCCGCGGGAAA
ATTATCATCCTGAATCACAATGGGCACTTTGGGAGATTTGAATTTCCTGCTTTCTGTGTGATCTTCAGTTTTTTTTCCAGACGTCTATTTCCCC
GCTGTCTGCCCTCTCCTCCCAGCTCTCAGCAACCTCCTCATATTCCATCTTACTCGCCACTTTCCAGATCGCTTGCTTTCTTATTTCCACATTT
TCTCTGACTTTTGCAGTATTCAATATTATTACCTGATTCTGATTTTTCAGACATCACTATATGGTCCTTGGCTTTTTGGTCGTTCCTCTCATTC
CTTTCCTGTTTCAAGCTCTGTTCCCAGCCTAAGCTTTTTACCACCGAATTCTGTTCTGTGACCCATAACTTAAAACTAACCCAACTTTTCATTC
CCTCCAGACTTTTTTTCGTAAGCAGTATCATCATGCTTTAGGTGGACACTGAGTCATACTGCTGTGGTAGTGCTGCTACACGTAGTTTTGAATA
CCAGGTTCTTGAGTGAATCCTTTTTACTTCTCTCTCTCTCCCCCACCCCCGCTTCTCCCTTCTGCCTTCATTTCTGACCCTTCTTTTCTGGTTC
CATTAGAAAAGGACTAATCTTATCAGTTCTGCCTTTTTTTTTCTGCTCTAATAATTTTGTCAAGATAATTTTTTCAAAGCACAAGGCCAATGAC
AAGGGGGAAAGTCGGTCCAGAAGGCAGCCGGCCCGACTATTCTGAGTTTGAATCTACTCTTCGTGGTCCTGTTAAAATACAGTTTGAAGCAAAT
CACTTGTTTTTATGCTTCTAATGTCTACTCTGTCGAAAGCCCAAATTTCCTTGCTTGGCATTTAAGGAGACTCCAAGTTCTTAAAACTTCTTCT
TGACTTTCATGCAAGTCAAACGGATTTATTTATTGTATCTCAAACAGTCCTCTTATATTTTCCTTTCTGTATTTTTTGATAATTTTCTTGTGCC
TGCAATATTCTCTGCTTTTATTTAAGTCTCACCATCTTTCAACACACAAAATATGTGTGATCTCTCAGGCCTTTGTGAACTGCCTCCAGCATTT
ACTAAGCAATTAATTCTGTCTCTGAACTTTTATAGCACAAACCCATTCGTTCACTTGATATTTAACAAATATCAACCTCTATCACTTATCTCTT
TTTTTTTTTTTTTTCTTCTCAGATGGAGTCTTGCTCTGTCACCCAGGCTGGACTGCAGTGGCGCGATCTCCCCTCACTGCAACCTCTGCCTCCC
GAGATCAAGCGATTCTTCTGCCTCAGCCTCCCAAGTGGCTAGGATTACAGGCGCCCGCCACGGGGCCCGGCTAATTTTTGTATTTTTAGTAGAG
ACAAGGTTTCACCATCTTGGCCAGGCTGGTCTCGAACTCCTAACCTCGTGATCCACCCACCTCAGCCTCCCAAAGTGCTGGGATTACAAGCGTG
AGCCACCGTGCGCAGCCTATCACTTATCTTTTCATGCTTACATTTTCTCACTGCTTGAGATTTTCTTTCATCCAGGGTCAAGCACTGACTCTAT
TAGAAACCCCACTGTATATGATCTGATGAAGATGACATTATCTTTTTAAAAGCCACATATTAAATCTCTTAAGTTAATAATATTGGCAATCAAC
TTAAATCAACTGAGTGACCACTATGTTTAAGGAAGTTTCCTTTCTGGTAGGCTGGGAACCGCGAGAAGGGAAGGATATAACTTTATAAAAAAAA
AAAAGCTGCAGAAGGGAAGGATATAACTTTATAAAAAAAGGTCCTTGTTCACAAGGAGCTAATCACTTACTGGCCACACAACAAACGTGTTCCT
TCTTTGCAATGGCTGTGTTTTTGGGTTTAGCTAACTCAATTCTTTATGTCCTCAATTTATGAGACCGCATCAAAAACGTTTTTGCCACATCTCT
GTGCGAAAGTAGGTGAGTCAGAATGAGTGCATAATATATAAGTATATATTTTAATCATAATGAATGTGTAGATTAACAAACCCAAACTTTGTTT
TTGTTTTTTTGAGAGTCTTGCCCTGTCGCCCAGGCTGGAGTGCAATGGCATGATCTTGGCTCACTCCAGCCTCTGCCTCCCGGGTTCACAGGAT
TGTCCTCCCTCAGCCTCCGGAGTAGAGTAGCTGGGATTACAGGTGACCGCCACCACGCCCGACTAATTTTTGTAGTTTTTAGTAGAGACGGCGT
TTCCCATGTTGGCCAGCTTGGTCTTGAACTCCTGACCTCAAGTGATCCACCCGCCTCGGCCTACCCAAAGTGATGGGATTACAAGCGTGAGCCA
CCGCGCCCAGCCAAACCCAAACATTCTAAAAGCGCAGAGTAACCGCTCCCCTCCCCGCCAACGGTAACCAGCATCTTCGTTCTCCAACCAGAAC
AGCTGTATGCACCGGTGTATAAGCCTGCCTTAAGTGCTATTTTTAATCTTTTTTAAAAACAGGCCTTAAAATAACTCATTTGGGTACAACTCCA
GCGGCTGGTGGAGTGGGCTTAATATATTAAACAGCTGTTAATTTTGGTTTCTTTTTGGTAACAGCCACACGGAGATCCGCATTTGTCGCAGTTT
CCACCGCGGGCGGGAAGTGTAAACACAAGAAATACAAACATAGCGCAACGGCTAAAGGAGTGTCACAGCGCCAAACCTTGCTGGCTCCGCGCCA
GGCCGAGCCCCACCCTCTCAGCTCGCGGCCAACACCCCCACCCCGCCTCACAGCCCGCCGCGCCCGCGGGGCGACACCCCCCACTCCATCATCC
CGGAGGTGGTGCGGCCGAGCCCCGGACCCAATTGGCGGAACCGCCGCCGGTTGTGTGCGCGCGCACAAATGCCCGGATGCGCCGCGACGACCCG
CGCGCTCCCCTCGGGCGGTAGGGGGCGCGCACCGAGGCACCGCGGCGAGCTTGGCTGCTTCTGGGGCCTGTGTGGCCCTGTGTGTCGGAAAGAT
GGAGCAAGAAGCCGAGCCCGAGGGGCGGCCGCGACCCCTCTCACCGAGATCCTGCTGCTTTCGCAGCCAGGAGCACCGTCCCTCCCCGGATTAG
TGCGTACGAGCGCCCAGTGCCCTGGCCCGGAGAGTGGAATGATCCCCGAGGCCCAGGGCGTCCTGCTTCCGCGCGCCCCGTGAAGGAAACTGGG
GAGTCTTGAGGGACCCCCGACTCCAAGCGCGAAAACCCCGGATCGTGAGGAGCAGGTACTGGCCCGGCAGCGACCGGTCACTTTTGGGTCTGGG
CTCTGACGGTGTCCCCTCTATCGCTGGTTCCCAGCCTCTGCCCGTTCGCAGCCTTTGTGCGGTTCGTGGCTGGGGGCTCGGGGCGCGGGGCGCG
GGGCATGGGGCACGTGGCTTTGCGGAGGTTTTGTTGGACTGGGGCTAGGCAGTCGCCGCCAGCCAGGACCCCGCCATTTCCCACCGCTCTCGCG
GCGGTGGGGGTGGGGGTGGTTCCGAGGTCTCCCCGGGAGTTCAGGGTAAAGGTCACGGGGGCCGGGGGCTGCGGGGCCGCTTCGGCGCGGGAGG
TCCGGATGATCGCAGGTGCCTGTCGGGTCACTAGTGTGAACGCTGCGCGTAGTCTGGGCGGGATTGGGCCGGTTCAGTGGGCAGGTTGACTCAG
CTTTTCCTCTTGAGCTGGTCAAGTTCAGACACGTTCCGAAACTGCAGTAAAAGGAGTTAAGTCCTGACTTGTCTCCAGCTGGGGCTATTTAAAC
CATGCATTTTCCCAGCTGTGTTCAGTGGCGATTGGAGGGTAGACCTGTGGGCACGGACGCACGCCACTTTTTCTCTGCTGATCCAGCTAAGCAC
CGACTTGCTTGTAGCTTTAGTTTTAACTGTTGTTTATGTTCTTTATATATGATGTATTTTCCACAGATGTTTCATGATTTCCAGTTTTCATCGT
GTCTTTTTTTTCCTTGTAGGCAAATCTGCAATACCAACATGTCTGTACCTACTGATGGTGCTGTAACCACCTCACAGATTCCAGCTTCGGAACA
AGAGACCCTGGTTAGTATTTTTGTCTCGTGTAACTTTTAAGAATAATTTATTTTATGAAGTGATAAACTAAATTTCGTATACCTCAATTGTAGC
ATGGCTCTGTAAATAAAATTGTATGTGCATAGCTTAAAGTGTGCTACCTCTGGCGTAACTGCGTGGAGTTAAAAACTGTTAAGAACTGGTCAGT
ATTTTTTTAGCCATTTGCCACATTTGCTTTAAAAATTAGTTCCATTACATTGTTAAGCGTGGTTGAAGTTACGTTTGTTACGTGACTGATTTTT
TTTTCCTTTTTGACCTCCCAGTATTTAATTTTTAAAAATGGTTTCTAAAAGTCAAAAAAATTATTCGTGCTTGTTTTAAAGATTTTTGTCATTA
GAGAAATTTTAAAGTAAAACCTGAAAATTCCAGGCTTTTTGCTCCATGTTTCCTTTTAGATTTTTTGTGTGCGTACCCACATATATTTACATTT
GAATTTAAATCTATGTTATTGTCCTGTTTGTTGTACTAACAAGATGAAACTGTAGTTGTTCTGTGACTTGCATCCTTGTCAATAAATAAAGTCT
ACAGAATTCTTTTTAATGGCTGCATAGTATTCCACAGCATGAGTATACCATTTTAAAAATTATTTATGCCTTTACTGATAGGTTTTTAAAAATT
GCCTCCTGTTTTTGAGTATTACAGATAGTGCCGCTTTGTACATGCATCCCGTGTGACTATTTCTGTAAGAAAGATTTTATAGAATTATAGGCTG
GCCTCGGTGGCTCACCCCTGTAATCCCATCACTTTGGGAGCCCAAGGCGGACCGATCACCTGAGATGAGGAGTTCAAGACTAGCCTGGCCAACA
TGGTGAAAACCTGTCTCTACTAAAAATACAAAAATTAGTCGGGCATGGTGGCGCGCGCCTATAATCCCCTGCTGAGGAGGCTGGGGCAGGAGAA
TCGCTTGAACCCAGGAGGCAGAGGTTGCGGTGAGCCAAGATTGCGCCATTTCACTCCAGCCTGGGCGATAAGAGCGAAACTCCGTCTAAAAAAA
AAAAAAAGAAAAGAAAAAGATTTTAGAGAATTAGAACTCCTGAGTTCTCAATTTTACTATTTGATTGCTACTGGTAACTTGCTCACCAAAAGGC
TCTAACAGTTTCACTCCTGCCAATACTGAATATCACCAATCTTTAATTATTATTATTATTTTTTTTTTTGAGGCGGAGTCTTGCTCTGTCTCCC
AGGCTGGAGTGCAGTGGCACAATCTCGGCTCACTGCAATCTCCGTTCCCCGGGTTCACGCCATTCCCCTGCCTCACCCTCCCGAGTAGCTGGGA
CTACAGGCGCCCACCACCACGCCCCCCTAATTTTTTGTATTTTTAGTAGAGACGGGGTTTCACCATGTTAGCCAGGATGATCTTGATCCCCTGA
CCTGGTGATCCGCCCGCCTCGGCCTCCCAAAGTCCTCGGATTACACCCGTGAGTCACTCCGCCTGGCCTCTTTTATTATTATTACTATTTTTTT
GATAAAAGGTACTGTACCATTGTTTAAATTCATTTTTCTCTAGTTTCTTGGTGAGGCTGGGCACCTTTCCTTTTTGTTTTTGTTTTTGTTTTTT
TTCAGACAGAGTTTCACTCCTGTTGCCCAGGCTGGAGTGCAATGGCGAGATCTTGTCTCATTGCAACCTCTGCCTCACGGGTTCAAGCGATTCT
CTTGCCTCAGCCTCTGGAGTAGCTGGGATTACAGGCATGCGCCACCACGCTCAGCTAATTTTGTATTTTTAGTAGAGATGGGGTTTTTCCATGT
TGGTCAGGCTGGTCTTGAACTCCCAACTTCAGGTGATCTGCCTGCCTCGGCCTCCCAAAGTGCTGGGATTACAGGCGTCAATCACTGCACCAGG
CCTCCATTTTGTTAAGTCATTTATTTCTTCTGGGACTTGCCTAGTCTTATCTGTCCATTTTCCTTTTAGTTTTTTAAAATTTTTCTTAGTAATT
GGAGTAGCTCTATGTGTTTCAAGTATATTTAACTTTTGTTGTTTACTAATGTAGCATTTAATTTTATGTTGTCTTTTCCCCATTGAGAACAATT
CTGTGAAGTAAAATTTGTTGGCTTTTGAATTTCATATCATGCTTTGGAAGAACTCTCCTGTCCCAAGATCTTGCAAATATATTCCTATATTTTC
TTCTACATTTAAATTTTTTAAATATTTAGATCTTCACCTGTAATTTACCTTCTTGTTCTTCAATGTAAGATTACAAATGCTATTATCTTTTATA
GCTGTTTTCTCATTAATACATGTCTTTTAAGTCACTAATATACTGATGCCTTTCCATTACAGATTTTTTTTTTTTTTTTTTTTTGAGACTGAGT
CTTGGTCTGTTGCCCAGGCTGGAGTGCAGTGGCATGATCTCGGCTCACTGCAACCTCTGCTTCCTGGGTTCAAGCAATTCTCCCGCAGCCTCCC
GAGTAGCTGGGATTACAGGCACCCACCTCCACACCCGGATAATTTTTGTACTTTTAGTAGAGATAGGGTTTCACCATGTTGTCCAGGCTGGTCT
TGAACTTCTGACCTCCTGATCTGCCTGTCTTGGCCTCCCAAACTGCTGGGATTAAAGGCATGAGCCACTGCGCCTGGCCCACACCTGGGTAATT
TTTGTATATTTAATAGACACGCAGTTTCACTGTGTTGGCCAGGCTGGTCTCACACTCCTGACCTCAGGTGATCTGCCTGCCTCGGCCTCCCAAA
GTGCTGGGATTACAGACCTGAGCCACTGCACCTGGCTTCCATTACAGATTTTTACTTTGAATTATTTTATCTAATTTGAAGCTAATATAGCATA
TAATAAAAATTCAGGAGAAAAAAGGTTATTTTTAAATCTTTTTTGAGAGAGTCCATTCTTTAGCCTGAAAGTAAAGTGCATATGTAAGCACAGT
TAAAAAAAAAAAAGTGATCAGAAAAATTAATAATTCAATTCTATATGTATTTTAAATTTTTCAATTCTTAAATAATATTTGTTTTATTGATTAG
CTGGAAATAGGCAATTTTTAAAAATCTGTTTTTAAGGCATCCCCAATGGGGGTGCTGTCAGATTTTAAAGTTAGACAGAGGACCGGCGCATATT
AAAGAGAGGTGCAGAGGTACCAGTTTGTGTGGTAGTGGCAAGCTGTAAAGATACTTGAGTTTTGCAGATTTTGATAAACCAAAAGGAAGGGAGA
AAGTGCATAGTATATTACATTCCCCTCCCCCATGACAATTATTTTTCTCAAGACCATTTGTAAAACCTCTAGATAAGGGGCAGGCAAATGTTAG
CCTGTCAGCCAACTTTGGCCTGTTACCTGTTTTTGTTTTAGTTTTTAAATTTTTTTTGTAGAGGTGGAGTCTCTCAGTGTCGCCCACGCTGGTC
TTGAACACCTAGCCTCAAGCAAACCTCTAGCACTGTCTCCCAAAGTGCTGGGGTTATAGGCAAGAGCCACCTCTCCCAGCCAGCTGCCTTTCTT
TCTGTAACGAAAATTTTACTGGAATGCAACCATGCCCGTTGGTTTACATAGTAGCCATGCTTTTTACTTGGATAGAACCATTTGTATGAAAAGC
TTAAAATACTTACAGTCTGGCTCTTTACAGAAAAAAATTGACCCCTAATCTAGTTGGCAGGCTCCTTGCATACAGGTCATATGTCTTATTTGTA
ATTTCTTTTGCAGTACCTACCACTGTCCAGTGCATAGTAGAAGCTCAATAAATCTTTCTCAAGTAGATATTTGAAATCTACACTAACTAAGAGG
CAGCATGATATAGTAGGAAGAAAATGCTGAGTTGTAGTTGAGGGACCTGGGTTGTTATCTCTGATCTGTTCTTAATTACCCTTGTCAGCTTGGG
TTAGTTAGTTAGGTTCTCTAGCCTTCAGGAACCTAATCTTTAGAAAGAGAAGATTGATAACAGGACCCACTCTAAAGATTTATTCATTTCTCTT
TGATTCTCTAAGTTAGCTTGCAATAAGCTCGTTGGATCAGTACGGTAAGCTCATTGGATCTCAGTAATTTATGGTTGTTTGTAAGGCAGTAAGC
AGGTAATATAAATAGCTGAAGGAAAAAGTAATCTTTGCTCTTTTGGATTGGATACTATCTGTGATCACAACTGATACTTGATGATTCCATGATA
CTTGCATAATCATTAGATCCTCCCCAGCATTTTTTCCAAATCCCCTTTATTGAACTTGATGGATATGTTTGCTGCAGGGCCTATAGTTCTGGGA
TAATTTTGGAAGTATAATAGCAGTTCTTTTCTCTTTATAGGTTAGACCAAAGCCATTGCTTTTGAAGTTATTAAAGTCTGTTGGTGCACAAAAA
GACACTTATACTATGAAAGAGGTAAGCTGAATCAAGAGATAAGTAGTATCTCACTAGTTACATGTAGCCATACTTAAAGTTTTCAAGACCATGT
GGAGAAATTGGCCATATAGAAGGATTTTTCATTAAGCAGCAACATTTAATTTTGTGGTCGTTTGGACTATATTTTAAAGCTGAAAATTCTAATT
ATACTCGAGTTTCATTTCTCAGCTAGGAACTCAGATAAATGGAAGCCCCGTAAGGGTGCTTGACCATTTTATGCTGTCAACCCTTTGGTAGACT
CCTTCCCAGAATCATGTTTTTAAATGTATCAAATAAAATGTATACAATTACAAAGGAAACATACTAAAATACGGTTACCAAAATAGTAAACTAG
TTTGTGATATGTGTGCTTTTGTTATTAACATAATAAATACTAAGATTTAGAAGAAGATATAATAAATATAATGTTTTTTTGTTTTTTGAGACGA
AGTCTCGCTCTGTCACCCAAGCTGGAGTGCAGTGGCACGATCTCAGCTCACTGCAACCTCCGCCTCCTAGATTCAAGCCCTTCTTCTGCCTTAC
CCTTCCAAGTAGCTGGGATTATAGGCACCTGCCACCACGCCTCGCTAATTTTTTGTCTTTTTAGTAGAGATGGGGTTTCTCCATGTTGGCCAGG
CTGGTCTTGAACTCCTAGTCTCAGGTGATCCACTTGCCTTGGCCTCCCAAAATGCTGGGATTATAGGCATGAGCCACCATGCCTGGCCAATAAA
TATTATCTTGATATAGTGAGGAATATAAGTCATATTTTGAAATGTTTGCCATAAGTACAGTATGATAGGAAAACACCTCTGATTTTTGTTGGTG
ATGAAATCACAGGTATTAATACTGTTGTGATTTGTTGCCTACATTTATAATCGAAGGAAAAGCTAAATTCTAGTTAGAAGTTTGTGAAAATAAG
AATAGAATTTTTTTTTTCCCATCCAAATTAATGGACCCCCTGAATTCTATCCAAGGACTTCTTGGGCATCCCTGGATCCCAGGTTAAGAACTTC
TGCACTAGAGATACATGAGTACACTATACTGATCTTTCTGGGATAGAGGTGAGCTGATTCATCACCATATATTCTATCTAATGGTTCCAGCATT
TTAATGCATTCCCCACCTTTAAATTTGTGCACAGAAGCATGATTTTCAGTGACAGATTGAGCCACCTTAGGCAGAATTTCAGTGTGATTGAAAT
ATGCCTGTTCTCTGTCTCTCCCACTTAGTTTATGGGACTCAGATTACTTAAAACATTTAAGTTTCTTTCTTACAACGGTATAGTGGGGCAGGAA
GAGCTTCCATTTTTCTTTTTCCTCTCCAGCTATACTTTTAGAAACTTCCTTAAAGGATGTTTGGTAGAATAAATCTAAGTTCTCTTCCTTTTTC
CTTTCCCACTGCTGGACAGCAATCTGTCCAATCCACAGTCACATAAGAATATTCCTTTAAGGCCGATGCCTGCTTAGCCAGTTAAATCCAAAGA
CAGTGTTTGTAGGAAAGGGCCAGGTTAAATGGTATTTTTAAGGCTAATTTGGGATTGACCTTATTATCATTGGATAATCAGTTAAGGAAACAAA
AAAGTATGGATTTAGCAGTAGATAGAAATGAGAAAATTGGCCAAGGCCATCATAAAGACTTAGAATGCTTATGCTAAACTATGTAACAGTAGTG
TATGTATTGTCTGATATAGTTCTCAGTTATAGGATCTTTGTTCTGGGGCACACAGGAAGGTAGTGAAAGCTAGTATGGGTAGTAAAAAGAGCGT
TGGATTAAGAGTCAGAAGAGCTGGGGCCAGTTTCTTCTTCTTTTTTTTTTTTTTTTTTTTTTTTATGTGACAGTTTCTCACTCTCTTGCCCAGG
CTGGAGTGCAGTGGCATGATCACTGCTCGCTGCAGCCTGGACCTCGTGGGCTCAGGTGATCCTCCCACCTCAGTGTCTGCAGTAGCTAGGACTA
CAGGCTTGTACCACTATGCCTGGCTAATTTTTGTATTTTTTTGTAGAGATGGAGTTTCACTATCTTCCCCAGGCTGGTCTGGAACTCCTGGGCT
CAAGGGATCTGCTTACCTCGGCCTCCTAAAGTGCTAGATTTACAGGTGTGAGCCACTGTGCCCAACGCAATTTCTTCATTTCTAATACAGTGAT
AATAATCTTATGAAATATCAGAATGAGATAATACTTATAAAAGTACATTGTAAACTGTAAAATGCCATATGTATTTAGGTAGTTGTTAATAGGT
TTATATCTGGATATCTTAAAAATGTATTGCCAACTTCTTGTCTTAATGCAGCACAGGGAATATAATTTAACTTTTATTAATGGTTCAAACTCAT
TATTTTGATTATTTATCATAATATGGAGTAAATTGATACCTTAGAGGGTTTATTTCATTCTCAGAATTCTCTCAAACTGTTTTGTAGCTGTCAT
TGATTGATTGTTTTTCTGTCTTCCTCCTCTGGTGACCTCAGAGGCTATTGAGGTTTATAGTATTTATGACTATTTACTAAGTGGCTCCAGACCA
CTGCCATATTTTAGTTGATTTTTGTTTCTGCAACTTTTTTGGCTATCAGTTTTTTATTTTCCTGCCTTTTCCCTTCCTAATATGTTAGTATCTA
AGTATCTGGGCAGAGTTAAAGTCTACAAGGAAAAAAACCTCACATTTTAACGTTTGCTTAAGTAAATAGAGAAGCAGTTATTTCTGCTTCTGTC
AAATTAAATGGAAAAGGCTTAAAAGTAGCTTTTTTTTTTTTTTTTTTTTTTTTTTTCAGATGAAGTCTCACTCTGTTGCTTAAGCCGGAGTGCA
GTGGCGCAATCTCGGCTCACTGCAGCCTCCACCTCCTGGGTTCAAGCAATTCTCCTGCCTCAGCCTCCCTAATAGCTGGGATTACAGGTGTCTG
CCACCATGCCTGGCTAACTTTTGTATTTTTAGTAGAGATGGGACCAGGATGGTCTTTATCTCCTGACCTCGTGATCCATCCCCTTCGGCCTCCC
GAAGTACTGGGATTACAGGTGTGAGCCACTGCGCCCAGCCAAAAGTAGCTTCTAAGTACAAAATGGTTGTTCTACATAGTTCTGGATATGGTTC
CTGGTTGTTTACCCCTATTCAGATAAATAGGATATTATTAAATGTATTTACAACTAACATTTAGTTTTCTTTAATGCTCAGAAATCATATTTGT
ATTTCAGGTTCTTTTTTATCTTGGCCAGTATATTATGACTAAACCATTATATGATGAGAAGCAACAACATATTGTATATTGTTCAAATGATCTT
CTAGGAGATTTGTTTGGCGTGCCAAGCTTCTCTGTGAAAGAGCACAGGTAATTCTTCAGTTTAGTCCATTGTAAAAAGCCATCTGGGCTAACAT
TTCAGTTCACCTCTACCCTCATTCACTTTTGTCAGAGAAAAACTGTTCAAACATATTAAAGACATGCTGAAACTTAATTTTTTAGCTCTGCGGC
ATATTATTTGAGAACCTGAGCTCTGGCATCTTATAAACCAGTGTTTGAAACCTAGCCCAATCATTTACTAGTTTTGTGACTAGATAAATTAACC
TTTTTAAACCTGCTTCCCCATTTATAAAATAGGGATAATTAAATACTTCATGTGGAGGTTAAAGCTCTTGAAGAGAGACGTTGTATAAAAAAGC
ATAGTTTTGGGCACATACAAAGCATTCAACTATTACCTGTACCTATTATAACTTTTTATTCCAGAAACAGTATACCACCACGTGGTAAAGCCTT
ACATTTGAGCCTTAGTTATTTTGGAGATCATTTTCATTGTCACCTATCATTATGGCACTTAACCAGCAGAATTTAACCCAGGTAATATTTCTAG
AAATAGGCCTGGTGCGGTGGCTCACACCTGTAATCCCAGCACTTTGAGAGGCCAAGGCGGGTGGATCACCTGAGGTTGGGAGTTGGAGACCAGC
CTGACCAACATGGAGAAACCCTGTCTCTACTAAAAATACAAAATTAGCTGGGTGTGGTGGTGCATGCCTCTAATTCCAGCTACTCGGGAGGCTG
AGGCAGTAGAATCACTTGAACCAGGCAGGTGGAGGTTGTGGTTGAGGTGAGCCGAGATTGCGCCACTGCACTCCAGCCTGGGCAACGAGCAAAA
CTGCATCTGAAAAAAAAATTTTTTTAATATATATTTTTCTAAAATTATTCTTTTTTTTGTTTGTTTGTTTTGAGAGGGAGTCTTGCTCTATGGC
CCACGCTGGAGTCCAGTGGCATCATCTTGGCTCACTGCAACCTCCGTCTCCTGGGTTCAAGTGATTCTCCTGCCTCGGCTTCCCGAGTAGCTGG
GATTACAGGTGCCTGCTGCCATGCCTAATTTTTGTATTTTTAGTAGAGACAGGCTTTCACGATGTTGACCAGGCTGGTCTTGAGCTCCTAACCT
TCGGTAATCCACCTGCCATGGCCTCCCAAAGTGCTGGGATTACAGATGTGAGCAACCACACCCGGCCTACATTTCGAATTCTTTGTCACATTTG
GTTTAGCACAGGCAATCCGTGTTGAGCCTTAGCATTCATTACATTAATAAATGGAATGAATACGTTCACCTGAGACAATTAACTAGCTTCTGCT
AGTATTTAATGCTCAGTAGTATAGCTGAAAAGATGATAATTAAATGGGATTTTATTTTATTAAAAAACATAACTTCCTCAGCTGAGCAAGGGAT
TTTATAACTTTAAAACAACCTTATTATTGTAAACATAGTATATATTATTATTGAACATTAGGAGATGACAGAAAACTAACAAAAGTAAAGGAAA
AAAAACGACTGTAGTTTGTTAACAAAAGGTATTTTTACCAGTCATTTGTGTATTATGTGCAACTGTGTGTTTTAACCTAGTTATGAATATAGTA
CTATATTTAAATTTCATCTAGCTTCTTAATTTGTATTATAATTTGTATATTATATATTATAATATACATATAATATAATTATATATATTACAAA
TATATGTATAATTATTATTTTTTATTGATGAGTCACTTCTGTTTAATATCCATATATGTTGTCATTTTATACGTGGCCTTGGTATATATGGTTT
AATTTTATAAATTTTAAAAATAACTATATTGGTATATTGGTGATTTCTTTACCTATTGAATATTGAATCTTCTATTTTTTTTCTTTTTTTTTTG
AGATGAAGTCTTGCTCTGTTGCCCAGGCTGCAACCTCTGCCTCCTGGGTTCAACCGATTCTCTTGCCTCAGCCTCCTGAGTAGCTGGGATTACA
GGTGCACACTACTATGAGCAGCTAATTTTTTGTATTTTTAGTAGAGATGCGGTTTTCCCATGTTGGCCAGGCTGGTCTCGAACTCCTCACTTCA
AGTGATCTGCCTGTCTTGGCCTCCCAAAGTGCTGGGATTACAGACGTGAGCCACTGTGCCTGGTCTGAATATTGAATCTTCGGATAATGTTTGG
AAGTATCATTTCCTTCCACTTGAATTCTAAATGTTGAGAGATAAGAAATAGGAGAAATTACTGACTTGAAAAGGATATGAATTAAAAATATTGT
GGGTTTAGGTTGCTGTATCCTTATTCCCAAGGATGAAAAAAGATAGTTGGGATTCATATTTCCTCTTAATATTGAGTGAGAAACAATATTTTGT
CACAAACACTCTCTTAAATCCTTAGTTTAAAAACAAAAGGAAAAATCCATTGTGACATCAACGAATACTGGGGTAAATGAATTATCCTCCTCTT
AGGATATAAACTTCATGAGGGCATATACATTTCTGTTTCATTCTTTGTTTTCCCCCGTGTCTGGTACATACTACCAGTGACTGGTACGTGGTAG
GTTTTCAGTCTGTATTTATTGAATGAATGAGTACTGCTTTATCTGTTAATAAGTTTCTTTTTTTTCTTCCCTGAGTCGGAGTTTTGCTCTTGTT
GCCCAGGCTCGACTGCAATGGCCTGATCTTGGCTCACCGCAACCTCTGCCTCCCGGGTTTAAGTGATTATCCCGCCTCAGTCTCCTGAGTAGCT
GGGATTACAGGCATGCGCCACCATGCCCAGCTAATTTTATATTTTTAGTAGAGATGGGGTTTCTCCATGTTGGTCAGGCTGGTCTTGAGCTCCA
GGCCTCGGGTGATCTGCCGGACTCGGCCTTCCGAAGTGCTGGGATTACAGGTGTCTGTGAGCCACTGCGCCCGGCCTCTGTTAATACGTTTTAA
GATCAAGAAAGGAAGTTGAAGCTTGATGAAAGTCAAGATTATAATCAAATTCCTATTTAATGGATTTTGCGTAACTCAATAGTTTTTTTGGCAG
TTTGTATTTAATGGATTTTGCATAACTCAATGCTTTTTTTTGGCAGTTTGCTACTAAGCACAGAGCTAAAAACATTCCTGTTTGAAAATAAGTA
TGTTCAACCTCTTCCTGCTTGCATACTCATTTTTTGGGGGTCTTCTGGTAAAGTCCAAACAAAGCACATTTCAGGAATTGGTAGTCTGAAATCT
TTCAGCTAGAGAGACTTTAGTAATTATTAAAAGTAGCTGTACATAATCATTGTGCTACTAATTATTGTGCTTTTAAGTGCCTACAAGCTTGCCA
ATGATAAATTTGGTTTTGAATGTGTGCAGTAGTTCATACTAAGTATGTATGTAGAAGTCTGGTTAGATCCAGCTTAATACAAATTTTTATTCTA
AAATGTACATCTCTTGTTATTTTTTTTTTTTCTGTCTACAAGGAAAATATATACCATGATCTACAGGAACTTGGTAGTAGTCAATCAGCAGGGT
AAGTTAATTTTGAGCATCATGGATAAATACCATAAAAACGTTTTAAAGACATTTTTGTTTATGTGCATATGTTTTATAATTGTGATTCTCTTTA
AAAGTTGCTTTAATTAAATATTGTAATAAAAGTATGATAAATTTATTAGAAGTACATATGAACTAAAACCACATACTGAGGTTCTAATGTAAAG
TTAAATATCCTGTTAGATCAATTTAGACTTGGGATATCAGAGTTAGGATTTATTTTGAAAGGTCTGTAATTTCAAAAGCTCCTGTTTAGGAGTG
ATCTACCTTGAAAATGTATTGAAAGGCTTTGGGAAATAATATTGTTACCTAGGTAGGACCATGTGTAAGGATGAAGGATGAGTCTTTTTCTTTG
GTTATAGACAAAATTTTGTTTACCAATTTGTGGAAGTGGAATTTTATGTTCCTACTTCCGTTTCTTAACTGGTTATTTTTGGGTCACATCTCTC
TTTAATAAGCTGATAAAAGCATTGGGTTCTTGTTCCAGAAAGATGCACATGTATATATACATGGGTAAGTCAATAAGTGTTTTACAACCTACTT
TCCATGAGAAAAGCCCTGATTTATGTGCTTGCTGACTTCCATAGTATAATACTCTCATTTGAAGGCTAAAAACATGGAGTTGGGAAGAGATGTA
CACAGTTTGCTTTTTTTTTTTTTTTTTTTTTGAGAAGGAATCTTGCTCTGTCACTCAGGCTGGAGTGTAGTGGCATGATCTCCACTCACTGCAA
CCTCCGCCTCCCAGGTTCAAGCAATTCTTCTGCCTCAGCCTCCCGAGTAGATGGGACTACAGGCGTATGCCACCACGCCCGGCTAATTTTTGTA
TTTTTAGTAGAGAGGGGGTTTCAGCATGTTGGCCAGGCTGGTCTCGAACTCCTGACCTTGTGATCTGCCTGCCTCGGCCTTCCAAAGTCCTGGG
ATTACAGGTATGAGCCACTGCACCCGGTCTCACAGTTTGCTTTTGAGTGCTGCTTAGAGCTGTCTCTAATACACTACACTGCTCCTAAAGCCCA
TCCTCGAACTACAGTTAAGAAATGCTCTTAGGCCAGGTGTGATGGCTCATGCCTATAATTCCAGCACTTTAGGAGGCTAAGGCAGGAGGATCAC
TTGAGCCGAGGAGTTCAAGACCAGCCTGGGCAGCATGGCAAAATCCTAACTCTGCAAAAACTACAAAAATCAGCTTGGTATAGTGGTGCATGCC
TGTAGTCCTAGCTACTCAGGAGGCTCAGGCAGGAGGATTTTTTGAGGCTGGGAGGTCAGGACTGTAGTGAACTGTGATCATGCCATCCAGTCCA
CGTGACAGAGCAAGACCTCATCTCTTAAAAAAGACAAAAACAAAACAAAACAAAAAAACACCTCTCAGGAGCTTGTTTAGTTCCTAGAATGAAT
TGACCACTCCCTTGTACTTCCTCTATTCCATATTCAGATTTTATTGTAAAATTGCTTTGTTTTACTTGTGGGTTTTTTTGTTTGTTTTTGAGAT
CGGCTCTCACTCTGTCACCCTGCTTGAAGTGCAGTGGTAATCTTCGCTCACTCCATCCTCCACCTCCAGGGCTCAAATGATCCTCCCCCTCAGC
CTCCCAAGTTGCTGGGACTACACATCTGCTCCACCACGCCTGGCTAATTTTTCTGTATGTTTGGTAGAGACGGTGTTGCTATGTTGCCCAGGCT
AGTCTTGACCTGAGCTCAAGAGATATTCTCACCTCGGCCTCCCAAAGTTTAGGCGTTACAGGCATGAGCCACTGCGTCTGGCTGTTGCACTTGT
TTATATTTTTGTTACCTGTGTCCTATTCAGGTTTGCATTGTGCATCACACACTATAGTGTTTTATTAATATGGTATTTAATAAATGTGTTGAAT
TGAATACTTATTCTTCCAAAGTTAGCATTTAAAGCCCAGAAAAGTATCTCCATTTTATCCCAAAATAAAAGTAGTTATTAGGCTGATGATTTGG
CATTTTATTGTTTTAAAGCTCTTTTTTTTTTTCAGCTTTAGATTGTATCCTTTTTATTTCCTTTTTTTTTAATTATTATACTTTAAGTTTTAGG
GTACATGTGTACAATGTGCAGGTTGGTTACATATGTATACCTGTTGTTTTAAAGCTCTTTAAGAAATTGTGTGATAGCTAAATATACAATTGTA
GTATTTGTAGTAGTAGTTTGAGTTTTCTGAAGTATGTAACCCTGATGTATTCATTCCTTCATTTGTTTAAATAAACTTTGGCACATTACTAGAA
AAATTGTATTATTGAGATGGAAACTTACAAATTACTTATTTGCAGTCTGAGTTTGGTTTAGATATCAGTAGAATTAACTTCAGAACTCCTATTC
CATGTTCTCAACTTTGTCACTTTTTTTTTTTTGAGATGGAGTCTCACTCTGTCACCCAGGCTGGAGTGCAGTGGTGCGATCTCGGATCACTGCA
ACCTCTGCTTCCCGGGTTTAAGTGATTCTCCTTCCTCAGCCTCCCGAGTAGGTGGGATTACAGGTGCCTGCCACCACGTCCGGTTAATTTTTGT
ATTTTTAGTAGAGAAAGGGTTTCACCATGTTGACCAGGCTGGTCTCAAACTCCTGACCTCAGGTGATCCTCCCACCTTGGCCTCCCAAAGTGCT
GGGATTACAAGTGTGAGCCACTGCACCCACCCAGCCTAGGGCTTCCTTTTTATTACATCTTTTTTGTAGCATCTGTTTATACTAATCCAAAATT
CCTGGACAATATAACCTATTATGTACAACTTAAGAAAATTAGTGTAATGCCTTACCCATAGTATAAAGAGAAAATTAATCTATGATAATGCATA
ATTTCAAAATGTTAATGTCCAGGCATGAGTTCACTGTTAAGATGAAAATGTTTCCAAGGGGGGTAGTAAAGGGTATTTGATGATAATTGTCTGT
ATATCCCAACTCTGAAACTTTGACTTTTGAGCTCAAAGTCTGTATATCCAACTACAAATGAAACCTATGTGCCTGAATAACTCAGTTTGTTATC
TTCCCATCCAATCTGTTTATCTTTCTAAAGTTTCTCTTTTAGTGAATGGTACCACTCATTTATTCACCCAAGTTGGAATCTTGGAATCATCTTA
ACTCCTTTCTTCCTTGCCTTCTATATCAAATACCAGGTCTTGTTGGTTTTTACCTATTTAATTTCTCTGGAATTTGTTTTCCATGCCTTAGTTT
AGGCTTTAATAGTTTTTAGTTTGTATTATTGCAATTGTCTAATACTGGTTTTCCTGCCTCTCCCCTCTTTTTCTTATGTTTTGCTAGAGTCATT
TCTCACTCTTCTGCTTAAAATTACCATTTAGGGGTTCATTATTGCTCTCTGGATGTAATATTTTCTGTGAGCTAGAAATCAGTTTCATTTCTTG
GACAAAAATCCTTTTTAATGGTGAGTGGCTGCAACATCGCATTAAGGAAGATTCTGAACTATCTGCGGGGAACAATGCAGTGATAAGGGAATGA
CAGCATGAGTCCTGCTTTGGCTAGACCACAGGATCCTTTACAGTCTGAACCTGCCTACATTCCAGTAATAATCAACTGCTTGTGGTGTCCTGCA
ACAGGAACTGTTGTTAGTTTGCTTTTGGATACTTTTTCACATGGCCTTTTCTTGGTCTGGAATGGCCTTTCCTAGCCCTATTTTCATATTCTCC
TACAAAATATTTCCTGATTCTTTGTTTCTCAGACACTCCCTGTCCCTACCAAAATAATCACTCTCCTGTCTCTCTCCTAATTTATCTAACAGTC
TTTTAATGTCTTGATTATACTTTACTCTTCTACATTAAAGTTGAATTATTTTATTTACATCTGCAACATTTAGTATGAGGATGCCACCTGGAAA
ATACATGTTTAATGTTTATTGAAAGGTTAAATTGCATAAGGGTTTGTGTTAGACTGATAGCATATCTACTGAGTAGCGCCCCGCCGCCCCCCGC
CCACCACCAAGTTTCTGATCCTTTTTCTTTTCTCTCAGAATCATCGGACTCAGGTACATCTGTGAGTGAGAACACGTGTCACCTTGAAGGTGGG
AGTGATCAAAAGGTAATCTAAGTAAATTTCTCATTCTAATGTAATATTATTTGCAAATTGGAAAGGTTATTTACAACAAGTTAGCTTACTCGTT
ATGTTAAGTTTGTTGTATTTTATTTTTTTCCTAAATGCTTAGGACCTTGTACAAGAGCTTCAGGAAGAGAAACCTTCATCTTCACATTTGGTTT
CTAGACCATCTACCTCATCTAGAAGGAGAGCAATTAGTGACACAGCTATATATGAATATTTATTTGACGCATTCACACAGCTTTTTGATATTCT
TTCTCTAATGAAATTAGTGCTTTTAGACTTAATTAAATTGTTCCCTTTTTTTGGTTGAGATGAATTAACCTCTGAGTTTTTTTCATTCGTGATT
TTATTTGATTTACAAATTGCTTATTTAGCAATATTTTTCTGGCTGACTACAGCAGGCACAGTTTACTCTGCCCCACTGGAGTTTATAGTTAGCC
AACTCTCTGTTAAGATAGTAGGAGGTGGCTGGGGTGTGGTGGCTCACGCCTGGCTCACGCCTGTAATCCCGCCACTTTGGGAGGCCGAGGCTGG
CGGATCACGAGGTCAGGAGTTCGAGACCAGCCTGACCAACGTGGTGAAACCCCATCTCTACTAAAAAAAATACAAAAAGTAGCTGGGCCTGGTG
GCGCACACTTGTAATCCAGCTACTCAGGAGGTTGAGGAAGGAGAATCACTTCAACCCGGGAGGTAGAAGTTACAGTGAGCCGAAATTGCGCCAC
TGCACTCCAGCCTGGGTAACCGTGTGAGACTTCGTCTAAAAAGAAAGATAATAGGAGACAGGCATATAAACTCTATTTTCACCATTATTCTTAG
TCACCTTAACAGTTAAATTTTTAAATTGTTATTTTTAATAAATGGTAGTACATACCTATGAGTTTTAAAACAGAAAGTAAATAGCATTTTAGAA
ATGAAGAGATAAGGGCCGGGCGCACTGGCTCACGCCTGTAATCCCACCACTTTGGGAGGCCGAGCCGCGTGGATCATGAGGTCAGGAGATCGAG
ACCATCCTGTCTAACATGGTGAAACCCCATCTCTACTAAAAATACAAAAAATTAGCTGGATGTGGTGGCGGGTGCCTGTAGTCCCAGCTACTCG
GGAGGCTGAGGCAGGAGAATGGCATGAACTGCGGAGGCAGAGCTTACAGTGACCCGAGATCACGCCACTGCACTCCGCCTGGGCCACACAGTGA
GACTGTCTAAAAAAAAGACAGAGAGAGAAAATAGTTGACAGAGAGAAAATAGTTGAGAACAGTTAGTAGACGTAGTAGACGAGAAGGCTGTTGC
CTGAGGAAGTCGCACTAACTAAGACCGTATGATTCAGATGAAAGAGAATGTCAGATCAGTGAAACAGAATAAATGAACCCAGACCAGAATCTCG
TACATGCGGGCAGAACACAGGAAGGGCAGAGGTGTCTAGGGCAGAGGTGGAACTAGAACAAATGGTAGTTACTTGGGGAAAAGGTGAAGTTAGA
TCTGTACCTTATGCCAAAATGAATTTCAAATGAGTTTAAAAGTTAAATGAAAAATAGAATACAACATATTTGAAAGATAGTCACTTTAAATTTG
ACTGTTAATATCTGTATTACATAAAAAGTCTTCCCAAATCAATAAGGAAAACATTAAAACTTCAAATAGCAAAAACGGCAGACAGTTCACAAAA
ATTTCTCACAGTAAATACGAATGACTAATAAATATGGGGAGAGGGTGAATTTTGGTGATTTTTAGCTTTACAGATAGTAAAAAATGCCAAAAGG
GTGTCCTTTTGATCTATCAAATTACTAAAAATAAAATTTTTACTCATCCTTACTCATCAGTGCTAATAACTTGTGTATTAGCACTGATAAACTG
TTGGTCTGTAAATTCGTAAAAGTGGGTAAAAATTGATTAAATTTTTCGGATTATAAAAAAGCTTAGATCGCCTGGTGCGGTGGCTCACACCTGT
AATCCCAGCACTTTCCAAGGCCGAAGCGGGTCGATCTCTTGAGGTTAAGAGTTCGAGACCAACCTGGCCAACATGGTGAAACCCCATCTCTATT
AAAAATACAAAAATTAGCCAGGTGTGGTGGCAGGCGCCTGTAATCCCAGCTACTTGGGAGGCTGAGGCAGGAGAATCGCTTGAACCTGGGATGT
GGAGGTTGCAGTGAACCAAGGTCGCACCACTGCACTCCAGCCTGGGGTACAGAGTGAGACTCCATCTCAAAAAAAAAAAAAAAAGAAAAGAAAA
AAAAGTCTTAGAGTTCTTAACATTATCCTAGTAACTCCATTTCTAGTAATCTATCCTAAGGATGTAATAAAAAATTCAAAGATTTATATGTAGA
GATACTAATTACAGCATTATTTATAATTGCAGCCCGGCCAAGATCACCTTTTTAAAAATAGGGAACACAAACATGATTAAGAACTCACGTGCAG
AACTTGTGATCTCGATAAAACGTAAAAATCCCGGCCAGGTCCAGTGGCTCACGCCTGTAATCCCAGCACCTTGGGAGGCCGAGGAGGGAGGATC
ACGAGGTCAGGAGATTGAGACCATCCTGGCTAACACGGTGAAACCCCGTCTCTACTAAAAATACAAAAAAAATTAGCCGGGCACAGTGGTGGGC
ACCTGTAGTCCCAGCTATTCAGGAGGCTCAGGCAGGAGAATTGCTTGAACCCAGGAGGTGGAGGTTGCAGTGTGCCGAGATTGTGCCATTATAC
TCCAGCCTGGGGGACAAGAGTGAGACTTTGTCTCAAGAAAATGATAAAAAAAGTCCTTTCAGAAATCTGTGATACTATCTCATTTTAATTTTTG
TCATTCTCATGGGTGAAAAATGATATCATTTCACTTTGCATTTCTTTGATCCCTAGGAGGTAAGAGTTTTTTTTTCTATTACTCAAGTACATGA
CTCAAGATTTTTCATATGTTTATTAGTCATTTGTATTTCTTCTGTGACTTGCTTTTTCCTGTTTCCTTAAAAGAAAATACAGTCTATCTTGATT
TGGTAGGACTTTGTATAATATGTATACTATGGGTAATTTTTACTATATATGTTGCAGATATTATCTTCGAATTTGACCTTTGCCTTTTAACTTT
TGTTTGCTATTTTATTTTGTCATGTAAACCTTGACATGGTCTAATCTTGATAGTCTTTTTATTGAGGACTTTGCAGTATTATGAAATACTTCTC
CACCCCAGCATTATAAGAATATTTTGTTATAGGTACTTGAATGGTTTTGTTCTTTATATTTAGATCTTTAATCCACCTGCAATTCATTTTTATG
TCTAGTATAAGGTAGTATTCTCATTTATGTCTAACGGATTTGTCTAACATTCTGACCCATTCAGAGATAATTTATTATTTAAAATGAAATGGAA
ACTTTTTACTGGTTTTGATTTCTCTTTAAAATATAGGGTACTCAACAGCCGGGCTTAGTGGCTTAGGCCTGTAATCCCAGCACTTTGGGAGCCT
GAGGCAGGAGGATCACTTAAGGCCAGGAGTTTGAGACCAGTGTGGCCAACATGGTGAAACCCTGTCTCTACCAAAAATACAAAAATTATCCAGG
CATGGTGGTGTGCACCTTTAATCCCAGCTACTTGGAAGCTGAAGTCACAAGAATCACTTGAACCTGGGAGGCAGAGGTTGCAATGAGCTGAGAA
TGTACCACTGCACTCCAGCCTGGGTGGTCGAGTGTGACTCTGTCTCAAAAGAAAAAGTGTAGGGTACTGAAGAAATTGAAACATTAAGAATACC
ATATGAGTAAATTAAACACTTTGGCTCTTTTCGGAAAAAAACAGATGAGCTCTTATATTTTAAAGTTTGGTTTTGACAGAAAAAAATTCCTAGA
TTTTTTGGTTAATAAAAACTTTATTAGATAGATTAAATTGTGATGTATCGACATAAAGTTCTGAAAGAAGAATACAAAGCTTTTTAAAAATAGT
ATTCTCAGCCTAGGCAACATGGTGAAACCCTGTCTATGCAAAAAATACAAAATGGTGCATTCCTATGGTACCGTTCCTATGGTACCAGCTACTC
GGGAGGCTGAGGTTGAGGTGGGAGGATCACTTGAGCCCATGAGGCAGAGGTTGCAGTGAGCCGAGATTGTGCCTCTGTACTCCAGCCTGGGTGA
CAGAGCAAGACCCTTTCTCAGACAATAAATAAATAAACAAATAAATAAATACACACAGATAGTGTTCTGCTGTAACAGTTGGACAGATTCAATA
AAACAGTGTTAACTTTTGAACTAATTTTATTGAAACTAAGTTTCTGTACAAATAGGTACTCAAAACAGCTCAATTTTCTAAATCACAGTACAGC
AATTTATTTTTTTTCCTTACATATCCAGAAGAAAATTCAGATGAATTATCTGGTGAACGACAAAGAAAACGCCACAAATCTGATAGTATTTCCC
TTTCCTTTGATGAAAGCCTGGCTCTGTGTGTAATAAGGGAGATATGTTGTGAAAGAAGCAGTAGCAGTGAATCTACAGGGACGCCATCGAATCC
GGTAATGTTCTCATTTTAAGTAAGGCAAGACTCTTACTGTTCAAAGTCTAGTCCTGGCAGTCCTAATAAGGATACGGATAGAATGTACATGTGT
CTTACGAGATTGATAGAAAACACAGTGCTAAATTTTACTTTAAAATAAAACTACTATATTGATCTCCACCTTCAACCCAAGGTATTGTGAGAAA
TGTGGAAAATAGTGGAGTAACACCGGTATTGGTAAAATTAGTAGTAGTATTAGTAATTATCAATGTCACTGTATGTATTAGAGAATATTTGTGG
ACCACTGCACCAATAGTACAGTCAAATAAATTGGAGTATACAGTTGTTTGAGAATTTGAGAGTTACACTTTTTAAAAAAGGAACAAAACCAAAA
ATTTGGTTCAGTTAAATATTTTAGCATTTTAAGGCAAATTTGACTATTTAGAGTAAAAATATATTTTATTACAGCCATCAGCAAAAAAGTCAGC
ATAGTCTGAACTTAACATTTGCCAAATAGGTCTAGACATCAGGTAAGTTCTATTTACTTATGCTGTAAAATACAGGTTTATACTGGGCTGGGTG
CGGTGGCTCATGCCTCTAATCAGTACTTTCGGAGGCTGAGGCGGGTGGATCACCTGAGGTCAGGAGTTCAAGACCAGCATGGCCAACAAGATGA
AACCATGTCTTTACTGAAAATACAAAAATTAGCCAGGCGTGGTGGCGCATGCTTGCAGTCCCAGCTACTTGGGGGCCTGAGGCAGGAGAATCGC
TCGAACCCAGGAGTTCCAGGTTGCAGTGAGCTGAGATTGTGCCACTGCACTCTAGCCTGGGTGACAGAGCGAGACTCCATCTCAAAAAAAAAAA
AAAAAAATGCAGGTTTATTATTGCATGGATGACGGCAGAGTATCAGTACTTTCTCATTACTAATACAGACTTCCAAAATATTAGTCAGCAGCAT
TTCTTAAGAGATCTCACTGAAAGAAGTTTGGTAAAAAATTTTTTTGTTAACAAGAATGCTTGACAAATGCAATATTTTGTTTTGTATGTCTTTA
ATCAACCTTGTCCAACCCATGCCCTGTGGGCCATATGCCAGGATGGCTTTCAATGTGGCCCAACACAAATTCATAAACTTTCTTAAAATATTAT
GATTTTTTTTTTTACAATTTTTTTTCTGCTCATCAGCTATTGTTAGTGTATTTTATGTGTGGCCCAAGACAATTCTTCCAATGTCGCCCACGGA
AACCAAAGGATTGGATACATTTTTTGGTAATATGGCAGTGTCTCATGGCCATTATTCTGGGTATTAGTAATCATTGTATAGTACTCTAGAACAT
GTAGGAAGTGGAACCAATTGTGATAGTAATCCTAGTCTTTTACCTATTCTTGTTACTTCCTGTATTATCTTTTTTTCTCTTCCAAAGCAGTGAT
TGTATGAACGCATACCTGCCCTCTAGAGTTACTGTACCTAACGGAGCTGTTACTTATGTAGACCGCTAGATGGCATGATGAGCTCCTGCTCAGT
CTGAGAGCAGGTTCTTCATTTGTAGATATTTAAGTTTATACCAAATTCCATACAGTTGAGATGGCACTGCAGATACAGTGGTAGTCAAGGTAGA
TATGGTTCTTGCCTTCATTGGGAGATAGATCATTAATGACAAAAATGAAAACATTTAATAAATCCAGTGGAAAAAATAGGCTCTTGAGCGGAAA
TATATGTGATAAAATTGAATCCAATTAAAACTTGATGAGTATCCAACCTACAAGTTTGGTTAAAAAGAGAATTATTATACCCTATTTATTTATT
TATTGACATTGAGTCTCTCTGTCTCCCAGGCTGGAGTGCACTGCTGCAATCTTGGCTCACTGCAACCTCCACCTCCCCGGTTCAGGCGATTCTC
ATGTCTCAGCCTCCTGAGTAGCTGGGATTACAGGCTCCCATCACCACACCTGGCTAATTTTTGTATTTTTAGTAGAGATGGGGTTTCACTATGT
TGCCCACGCTGGTCTTGAGCTCCTGACCTCAAGTGATCTGCCTCCCTTGGCCTCCCAAAGTGTTGCCATTACAGATGTGAGCCACCTGCCCGGA
CCCCTATTTAATGTATAGTTTTTGTTAAAGTTATATCAGAATTCTAGCTGAATGATATTTAGAGTCCTTGTTCCTGCTTTGTACCTGGGACACT
GCCTTGTTACTGATATGGCTTTCAGCAGTAGAGGAACACCAGGTTTTTTCTTTTGAGTTGAATTAGATAAAACCACACGGACACACGTGGAGTG
GTTTTAAGGAGCAGACCGTTTAATAGGCAAGAAGGGAGAAGGCAGAAGGAAGAAGCTCCCCCTTACACAGACAGAGGGAGGGGGGCTCTAAAGC
TGAAAGAGGAGACCTTACATTCTGACAATACTAGCCACTTTTATGTGACGGCTGGAGGGGGTGCTGTCTTATTTGCATAGGGCTCAGAGGATTG
GTTTGACCAGGCATGTCATTTAGCCCTCAAGAAAACTGGCCCTCCCACCCCACTCCTTTAATACACAAATGCAGGATGCCATGACATTTTACAC
ACGGGGTTATGTGGGCGCGGCCATGCTGTTAGGTACCTGATGGAGCAAGGGCAAGAGGACAACGGTGGGAATCGCCATGTTGGGTGGACCCTCT
TGCATTTGCATCTTAAAGGTTGCCAGCCTTTAAGAGCCAGGGCTTTTATGCTAGACAAGAAATTTTTCTGGAGCTACGAAAAACCTTCCAAGGA
CCACTTTTCCTCTCCATTTGCCTAAAATAATTTCTTAATAACTCGTGTGGAGTAGAGGGGTATTTCTTTTTTTCTTTTTGTTAGTCCTGTCCTG
CATTCTCTTAAAAAATCCTATCTATAAAAAATCTCAGCCGGGCGTGGTGGCTCATGCCTGTAATCCCAGCACTTTGGAAGGCCCAGCCGGGCGG
ATCACCTGAGGTCAGGAGTTCAGGACCAGCCTCGCCAACATGTTGTGAAAACCCATCTCTACTAAAAATACCAAAAATTAGCTGGGCATAGTGG
CGGCCACCTGTAATCTAGCTACTCAGGAGGCTGAGACAGGAGAATCGCTTGAACCCGGGAGGCGGAGGTTGCAGTGAGCTGAGATCACGCCATT
GCACTGCAGCCTGGGCAACGAGAACGAAATTCCATCTCAAACAACAACAACAAAATCTCGTTTTTCAGCTTAAGTCCCTAGTTGTCAAAGCTTT
TGGTAGGGCCAGTTTTTTTTTCTTCTTCGTTTTAATTTCCCTCATTTGAGAATGTTGTAGTCATCATTGGGTGTCATCATTCACCAGTTTGTGA
AGGGAGTTAAAGGAAAGTGCATTCCTCTCCCATCTTTTCTTTCTTTGGGGTTTAATAAATTGTGGGAAACTAAGACTGTGTTCTAGACTGTACA
CCATGCAGTTTATATTATGATCTTCTCTCATCTTTGCAGCCTCAGATTCCATTTTTAGCTAAGACTGGGGTTTTAAATAACTTGTTTAAGGTTC
CTACAACTAATTGTCACAATTGGAATTCAAACAATTCTAGGTATTTTTGACTACAAAGATCTAAGATCTTTGCAGAGCAAGCCTATCTCATGCT
TAAAAAAATATATATTTTTTAAATTTTTGAGACAGTCTCTCACTCCGTTCCCCAGGCGGGAGTGCAGCGCATCATCATGGCTTACTGCAACCTC
AACCTCCTGGGTTCAAGCAATCCTTCCTCCTCAGCCTTGAGAGTTAGCTGGGACTACAGGTGTGCACCACCACACCTGGCTAATTTTTTTTACT
TTTTTTTTTTCACAGATGAGATCTTGGCCTGTTGCCCAGGCTGGTCTCAAACCGCTGGGCTTATGTGATTCTCCTGCTTTGGCTCCCAGAGTAT
TGGGATTACAGGCATGAGCCACCACCTCCAGCCTGATGCTTTTTAAATATATACTATTTAGGTCTGGTGCAGTGGCTCATCCTTGTAATCCCAG
CACTTTGGGAGGCTGAGGCGGGCGGATCACAAGGTCAGGAGATCGAGACCATCCTGGCTAACACGGCGAAACCCCGTCTCTACTAAAAATACAA
AAAATTAGCCGGGCGTGGTCCCCGGCACCTATAGTCCCAGCCACTGGGGAGGCTGAGGCAGGAGAATGGCGTGAACCCAGCAGGTGGAGCTTGC
AGTGAGCCGACATGGTGCCACTGCACTCCAGCCTGGGTGACAGAGCGAGACTCTGTCTCCAAAAAAAAAAAAAAAAAAAAAAAATATATATATA
TATATATTTATATTTAAATAATGTATAAATATATAATATATAATTATAAATCTATTATATATTAATGTATATTATATATTACATTGTTATATAT
ATTTATATAATTATATATTTATATAAATATAAATATATATATTTATATAAATATAAAAATATAATTATATAAATATAAAAATATATATTTATGT
AAATATAAATGGGCAGGCACCTGTATATATTATATATATTACTATTTATATAAATATAAATATATATTTTAAATATTTATTTTATATATTTTAA
AAATATATATATAGAGAGATAATTTAATAGTTTCTGGGGCATTCTTTTGCGAACATTAGCCAAAACTTTAAAAACCACTCCCAATATTGCATTT
TTTCATCGTATTTTTTATAGTATTTTTATAGTATCCTAGGACAGCAGTTGGCAAACTTTCTGTAAAGAGCCACATAGTTAATTATTTGGGCTCT
GCCGGGCCATCGTTTCTCTCACAGCTACTCAGCCCCGCTCTTGTAATCCACGAGCAACTGTAGACAACAACTCAACAAATAGGCATGGCTGTCT
TCAATAAAACTTTATTTATAAAAGTCGATGGGTGATGGGCCACAGTTTGCAGACCCCTACCGTAGAGTATCTTTTCTGAGATTTAATGTTTCTT
GTGATTTACCCCCTTATTCTAAAATTAGTGCACTAAAGACTTTAGTTAGTTGAAGTATTTGGTTTGTTGGGTTACTTTGTTAGTGTTAATTGTA
TCCTAACTTGACTTGTGATTCTAATCTGATTGGTAGTAGAGCCCTTGTTAGGAATTTGAGCTGCAACTTGATGAATGTTGCTTTTACTCTCCTT
CCTAACATTACTCTAAAAAAGTCTTTTGATGTCAGAGGCTGTAAAATAATATACTTTGTTTCTGTTTTTAAAACTGATACAGATATGGAAATCA
TTTAATCTAAGCTCCTTTTCTAGATGAAGTAGCTACAGCCTTTAGAAGTTAAATGATATGGGCTGGGCGACGTGGCTCACAGGTGTAATCCCAG
CATTTTGGGAGGCGGAGGTGGCGGATCACTAGAGGTCAGGATTTCGAGACCAGCCCAGCCAACATGACGAAACCCCGTCTCCACTAAAAACATA
AAAATTAGCCGGGTGTGGTAGTGGACACCTGTAATCTCAGCTACTTGCGAGGCTGAGGCAGGAGAATAGCTTCAACCCCGGAGGCGGAGGTTGC
AGTGAGCTGAGATCATGCCACTGCATTTCATCTTGGGGGACAGAGCAAGACTCTTGTCTCAAAAAAAAAAAGGGCCGGGCATGGTGGCTCACAC
CTGTAATCCTAACACTTTGGGAGGCCAAGGCGGTTGAATTGCTTGAGGTCAGGAGTTCAAGACCAGCCTGGCCAACATGGTGAAGCCCTGCCTC
TACTAAAAATACAAAAATTAGCCAGGCATGGTGGCATGCGCCTGTAGTCCCAGCTACTTGCGAGGCTCAGGCAGGAGAATTCCTTGAACTTGGG
AGGTGTAGGTTGCAGTGAGCTGAGATCAGGCCACTGCACTCCAGCCTGGGTGATGGAGCAAGATTCCATCTCAAAATAAATAAAATAAGTTAAA
TGATGTTCTTTAAGGTTAAATTAGCTACTTAGTGGCAAGTGTAGAATAAGCCATATCCCGACTTCTAGACTTTTTCCAATTGTAGCCTTCTGCT
TATATATGCTCCTACCCTTAGCCACTGACAGCAAATATTATTGCCCTGAAGGTCTTACTACCTTCTTATACAATTTATTGAAGTCCTGAAACGA
AGAATCAGATGAGCAGCTACAGAATTTTTTTTAATTGTCTAAAGGCTCTTTAAAAGAAAATCTCAAAGTTTATTAAAAAACATAAGTATCTATG
ACTCGAGACATTTTTCTAAGAAAAAAAACCCATAAGTATTACAAGTGTTGTGTCTTGTGACGTGTATTCTACCAATTTGGACATAGAAACATGA
GTGACAGGTTTTAGCTCAAAGTATATATAATTCAGATGAGAAACATGCCTGCAAAATTATGTTTTGCATAATTTATGTGTGAGACTATATAATT
CATTATTATACAAGTAGTAAGGATAATTAAAGGTGGAAGAGCCTTTTCAGTTATGGTATTGACAAAGGCTTAGTCAAACAAGGATGAATAGGAT
TTTGAAACTGAAATTATTCTAGTTAGAGGGGGAAAGTGTGGAAGTTGAAATGCCAAGATTAGTTGAGCAAGCAATCTAGATCTGGCGGGATTAT
CCATATTTAGTAAGGGAGGAGGGGTGTCTAGTTGAAGACAGACGAAGATGTTAATACAGTCACACTGTAGGTACTTTCGATGCCAAGAGGCTTT
GGTGTTTTTCCTTTATGCAGTGGGGTTTTGAACAGGGAAAATAATGTAGTAATAACCTTGAAACCTTAATCTAGTTTCAGTATGCAGGATGGAT
CTGAGGAGGAAATGTGCGTGGCAACCGGCTCCGGGATGGCTGGCTGGGGGGCCTAGTCTTCTGCCCGCCGATCTCCCTCGGGAAGTCCCGGATC
AGAGCAGCAGGCCCATCCCCCAACTGTTACACCCTGCTGGCAGCAGCAGAGGCACAGGGATGAGTTAGGAAACAGATACAGAGGTCAAGAGGTG
ATGTTTATTAAGTTATATATTTTTTTCTTGTTTTAGGATCTTGATGCTGGTGTAAGTGAACATTCAGCTGATTGGTTGGATCAGGATTCAGTTT
CAGATCAGTTTAGTGTAGAATTTGAAGTTGAATCTCTCGACTCAGAAGATTATAGCCTTAGTGAAGAAGGACAAGAACTCTCAGATGAAGATGA
TGAGGTAGTATTTTTTTTCCCCTCTAATTATATTGGAAAATTATTAAATATTTTCTATGTTCATTGACTTTGAGATTGAAATAATATTATTCAG
ATTTCACTTGAAATCTTTACCTCTTGATTTGTTTAAACTAACACATTGGTTTGTGGACTTGAGGATTTCATAGTTCTTTATCATCTGATTTATA
TTATTATATTACTACTAATTGCCAAGAAGTCAATAGACCTCAATGAACATGCTTATTATAATGGTCTGTTCACTTTTAAAAACATATAATTCAA
CTACTGTTTTAATATTAACTTACTTTCTATAAAAGTAAAACATGCATACAGTTAAAGTCAAGTAGTACCCTTAGAGATCTGTTTCCCAGAAGTG
ACTGCCTTTAACATTTCTAGCTATTTCCCTATTATTTAATATCTCCATATTATTTATTTATGAAGTAAAACCACCTAGCTGAATGTTTTGTTTT
ATTCATCCTAAACATCCTTTGTATTGACTTTTACCATTGTGGGTAAGGATTTCTCTCTCCTCCATTTTTTCCCCCTTTACACTCACTTACTCTA
TTTGATATTGTCTAAGGCTTTCTCATATATTGTAGTACATGATATTTGTTTAGGACTTATTACTAGGAAGCCTTCTGATTGAAGGAAATACGGC
GATGAATTGATGCTAATGAATGTGTTTTATTAGGTATATCAAGTTACTGTGTATCAGGCACGGGAGAGTGATACAGATTCATTTGAAGAAGATC
CTGAAATTTCCTTAGCTGTAAGTATACATCTACTTTTTTAAGAAATAAAAATTTCATTAACCTCAAGATTAGGAGACTATATCTAGCTTCTTTC
TGAAATGAACTGATTTTTATAAAGTTAAAATGTTTTTGTAAAGTCTGAATAATTTAAAGCATTTTTCTTTTATTTGTTGTGATGAGACCTGTAG
CTATAATTTTTATTTTTAGGTGCTGGAATTTGAACTTTTTTTTTTTTTTTTTTAAACAGGGTCTTACCCTTCTCCCAAGCTGGAGTGCAGTGGT
GCAATCTCAGTGCACTGCAGCCTCCACCTCCCAGGTTCAAGTGATTCTCGTGCCTCAGTCTCCTGAGTAGCTGGAATTACAGGCATGCACCACT
ATACCCCACTAATTTTTTTATTTTTAGTAGAGATGGGGTTTCACCATGTTGGCCAGGCTGGTCATGAACTCCTGACCTCAAGTGATCTGCCCAC
CTTGGTCTCTCAAAGTGCTGGGATTACAAGCATGAGTCACTGCCCAGCTGAATTTGTACATTTTGATGTTATTTTGGAAAATTTCAGTGTATCT
CCAGTATACTCATCACCCAGCTTTAACTATTACCAACATTTTGCCAACCTTGTCAAACGTAAACTTTTTTTTTTTTCAATTGAGATAGGGTCCT
GCCCTGTCTTCCAGGCTGGAGTGCAGTGATGTGATCATGGCTCACTGCAGCCCCCACCTCCCAGGCTCAAGTGATCCTCCCACCTCAGCCTCCT
GAGTAGCTGGAATAACAGCTGCATCCCATCACACCCCAGCTAATTTTTGTATTTTTTCTACAAATGTCACCATGTTGCCCAGGATGGTCTTGAA
CTCCTGGGCTCAAGTGATCCACCTGCCTCAGTCTCCCAAAGTCCAAGGATTACAGGTGTGAGCCATCACACCCAGCCAAATGTAAACATTTTTA
TTACTGATTTGTATTATAGAATGTGGTAGGATTTGCCATTAAAGAAATTAACACTTAAATGTTTACATCTCATGCATACTTCACTGACTTCCTG
AGGTATTATAGGTTTTTTTAAAACTATTGTGCTTTAGTTTTTGTTCTTAATATTGCCTTTATTATATTTGAATAGGAAATAAATTCCATTTTTT
CTGAGTTATAGACAGTTACTAATTATTCAAGTGCAAAAAAGGATGAAAATTCAAGAGTTTCCAAAATTTTTACTGTATTAGAAAATAAATAACT
AATTGAATCATTTGATGTCAAGTTGAGTAAAATATTTTAGATACTTTTAATTTTTTAAATCTATTTTAAGACTCAAATTATTTTAGATATTCAT
CTTGGAATTTATTGGACTTTTTGATGCACTTTTACTCCCATATTGCAAGACCTTTTTCTTTGGTTTTATGACCTGTGAAAAACCCCCTTCTATA
ACTTGAAAAAAGAAGAATGAATAGTCTATCTTTATTGCTCCTGTCTTAACATCATTTGTGAGGCAATCTCAAAATGGTTACGGAAATACCTCTT
ACTTAAGAGTTTTCACTTGTTAAATGTCCTCAGGCAAGAACCATGCTTCAGCTAAGGCAACAAGATAGGTTATGTAAAAAGGCTGGGCATGTGG
GAGAGTTTGTTATCCAAATTGCATGGTGGAAAGGTTTGTGGGATAAAACGTTGGGAAAAGGAAGAAGCATTGAGCAGTTACAAAGATGAGAACA
CAGGAAAGGATAGACAGGTAAAGCAAACGATATTACGGTTAGTGACCAAGGATGATAGGTTTTAGCACCAATACTTGCTGTCAAAAATGTTTTA
GCATTTCTCTTGTGGATGGAGTTAGGGAATACTCAGTTAAGCCATGGTTTTAGAGTACTTCTAAGGTAGTTCTAGTCACTGGTGGATTAGCAAG
TCTTTCTCGAGGAGGCAGGTTTCATCTAGATTGATGGGATGGACTCTTGGAATAGCGCCTAGGGTTGAGCCTTTAAGGGAGTGGATATAGGCTG
CATGACACAGTAAAAGATCCCATAGACTTGCTTCAACTAAAGGAAGCAACTGCATTTGTATCCTGAAACAGATAACTTTATAGGTACATGATTA
ATATCACATCACACTTAATATATTTTTAGTTTAGAATTTTTCAGGATGGTAAGGGTGAAAAAAATGGGTGTACTTACTCTACTTTAGGAGAGAC
TGTTACATATTGACCTTTAAAAATCTGTTTTGTGTATAATTGAGACATATAACGTGATGTTTTGACACACATGTAAATAGTGAAATGATCACTA
CAGTCAACCAAATTAATGTAATCCATTATCTTGAATAGTTAACCTTTCTTGTGTGTATGGTTAAAATACCTAAAATCTATTCTTTTACTAAATT
TCCAGTATACCAATATATAACTAACTATAGTCCTCATGCTGTTTACAGTGACTATTTTATATAAAACATGAAACACTGAATATTGAGCCCTATG
ATATACTTTACCTTAGACATAGCAAAGTTGCTAGCATTCCTGTGACTGAGCAGTTAAAGGGTTACAGAAACTGACTGTCTGTCTTATTTCATTG
AAGGACTATTGGAAATGCACTTCATGCAATGAAATGAATCCCCCCCTTCCATCACATTGCAACAGATGTTGGGCCCTTCGTGAGAATTGGCTTC
CTGAAGATAAAGGGAAAGATAAAGGGGAAATCTCTCAGAAAGCCAAACTGGAAAACTCAACACAAGCTGAAGAGGGCTTTGATGTTCCTGATTG
TAAAAAAACTATAGTGAATGATTCCAGAGAGTCATGTGTTGAGGAAAATGATGATAAAATTACACAAGCTTCACAATCACAAGAAAGTGAAGAC
TATTCTCAGCCATCAACTTCTAGTAGCATTATTTATAGCAGCCAAGAAGATGTGAAAGAGTTTGAAAGGGAAGAAACCCAAGACAAAGAAGAGA
GTGTGGAATCTAGTTTGCCCCTTAATGCCATTGAACCTTGTGTGATTTGTCAAGGTCGACCTAAAAATGGTTGCATTGTCCATGGCAAAACAGG
ACATCTTATGGCCTGCTTTACATGTGCAAAGAAGCTAAAGAAAAGGAATAAGCCCTGCCCAGTATGTAGACAACCAATTCAAATGATTGTGCTA
ACTTATTTCCCCTAGTTGACCTGTCTATAAGAGAATTATATATTTCTAACTATATAACCCTAGGAATTTAGACAACCTGAAATTTATTCACATA
TATCAAAGTGAGAAAATGCCTCAATTCACATAGATTTCTTCTCTTTAGTATAATTGACCTACTTTGGTAGTGGAATAGTGAATACTTACTATAA
TTTGACTTGAATATGTAGCTCATCCTTTACACCAACTCCTAATTTTAAATAATTTCTACTCTGTCTTAAATGAGAAGTACTTGCTTTTTTTTTT
TCTTAAATATGTATATCACATTTAAATGTAACTTATTATTTTTTTTGAGACCGAGTCTTGCTCTGTTACCCAGGCTGGAGTGCAGTGGCGTGAT
CTTGGCTCACTGCAAGCTCTGCCTCCCGGGTTCGCACCATTCTCCTGCCTCAGCCTCCCAATTAGCTTGGCCTACAGTCATCTGCCACCACACC
TGGCTAATTTTTTGTACTTTTAGTAGAGACAGGGTTTCACCGTGTTAGCCAGGATGGTCTCGATCTCCTGACCTCGTGATCCGCCCACCTCGGC
CTCCCAAAGTGCTGGGATTACAGGCATGAGCCACCGCGTCCGGCCTAAATGTCACTTAGTACCTTTGATATAAAGAGAAAATGTGTGAAAGATT
TAGTTTTTTGTTTTTTTGTTTGTTTGTTTGTTTGTTTGTTTTGAGATGAGTCTCTCTGTCGCCCAGGCTGGAGTGCAGTGTCATGATCTAGCAG
TCTCCGCTTCCCGGGTTCAAGCCATTCTCCTGGCTCAGCCTCTGGAGCAGCTGGGATTACAGGCATGCACCACCATGCCCAGCTAATTTTTGTA
TTTTTAGTAGAGATAGGGTTTCACCATGTTGGCCACGCTGGTCACGAACTCCTGACCTCAACTGAGGTCACCCGCCTCGCCCTCCCGAAGTGCT
GGGATTGCAGATGTGAGCCACCATGTCCAGCCAAGAATTAGTATTTAAATTTTAGATACTCTTTTTTTTTTTTTTTTTTTTTTTTTTGAGACAG
AGTCTTGCTCCATCACCCATGCTAGAGTGCAGTGGAGTGATCTCGGCTCACTGCAACTTCCGCCTTCTGGGTTCAAGCTATTCTCCTGCCTCAG
CCTTCCAAGTAACTGGGATTACAGGCATGTACCACCATACCAGCTGATTTTTTTGTATTTTTAGTAAAGACAGGGTTTCACCATGTTAGCCAGG
CTGATCTTGAACTCCTAAACTCAAGTGATCTACTCACCTCAGCCTCCCAAAATGCTGGGATTACAGATGTGACGCACCTGGCCTCAGATTTTTG
ATACTCTTAAACCTTCTGATCCTTAGTTTCTCTCTCCAAAATACTCTTTCTAGGTTAAAAAAAAAAAGGCTCTTATATTGGCTGCTATGTAAAT
GAAAATGTTTTTTAGGTTTTCTTGATTTAACAATAGAGACAGGCTCTCCCTGTGTTGCCCAGGCTCCTCTCGAACTCCTGGGCTCAAGAGATCC
TCCTGTCTTGGCCTCGCAAAGTGCTAAGTAGGATTACAGGCGTTAGCCACCACACCCGGCTGTAAAAATGTACTTATTCTCCAGCCTCTTTTGT
ATAAACCATAGTAAGGCATGGGAGTAATGATGTTATCTGTGAAAATAGCCACCATTTACCCGTAAGACAAAACTTGTTAAAGCCTCCTGAGTCT
AACCTAGATTACATCAGGCCCTTTTTCACACACAAAAAAATCCTTTATGGGATTTAATGGAATCTGTTGTTTCCCCCTAAGTTGAAAAACAACT
CTAAGACACTTTAAAGTACCTTCTTGGCCTGGGTTACATGGTTCCCAGCCTAGGTTTCAGACTTTTGCTTAAGGCCAGTTTTAGAAACCCGTGA
ATTCAGAAAAGTTAATTCAGAAATTTGATAAACAGAATTGTTATTTAAAAACTAACTGGAAAGATTGTTAAGTTCTTTCTGAATTATTCAGAAA
TTATGCATCATTTTCCTTCAAGAATGACAGGGTCAGCATGTGGAATTCCAAGATACCTCTTGACTTCCTCTCAAGCTCCGTGTTTGGTCAGTGG
ACGCCCATCCGAGCTCAGCACTGAGAAGTGTTAGTTTCTTTGGGACCCATCTACCCTGACCACATCATGATGTTCATCTGCAGCTGTTGCAAGG
TGTTCAGATTGTATAAACATAAATGTCACAAAAACTTTAAAAGAAGTGCAATTCTCAAAAGGTTAGGTGGACTAAAGCATTCTGTAAAGCAACT
GCTAATAATGAGCTTACAGTGGATTTGAATTTGAAAAATATAGTAACAAGCCTGTCAAATATCTGCAAGAACTATGGAATAAAACTACTGATGC
AGTGAAGACAGTTGAAAAGATCAAACAAATGCCAAGCTATATTTATAATGAACAAATTCAAGAAAAAGGACTACGGAAAGTTCAGGACATCAAA
GAAGTCAGGCAAAACTCATCTTGACCCCTGTTGCAGGCAAAGGAACGCAGCTGGAAGAAAAGATGATATAACAGTTAACAGGATGCAGACATGG
CAGAGGTTTCCTAAAAATCTCATTATCTATAACCATTTCTATATTTACATTTGAAAATCTCCTTTGGAGACTTAGAACCTCTAAATTATTGACT
TATTTTTTATATAAGGTCACTCCGATGAAAGGTGATTACAAAATCATCTACATTGCTGTCTACAAAACAGATAATATGGATGTTTGATCGCATC
TCATTGTTAACTCTTTACTGATATGTTTGTAAATACAGAAGTGAAATGTGGACATAAAATAGTTACGCTATTTGGTTAATGGTACTAGACAACA
TGTAATTAATGACATTCAAAAATTTATGGCTAGTGATATATATAAAGTAAAATTTTCTTTGCACTAAAATATGCCCTTTATTATAGAAGGGAGG
ATATAACGAACCAACACTTTGTATGAAAATAGCTCAAATAATATCTTTTATTTTGATTTTAATATTTCTTATTTTGGTTTATTAGTGTCTTAGA
ACAAAATGGCCTTATATAATGAAGCCTAGTTATGCTGGACTGTTTGGATCTCTTTTAATTGTTCTGACAGATAGTTGGGGATGAGAGCCGAATA
AGGTTTGCCTGALATAACTGACACTATATAATTTCTGCTTTGGCAAATACTAACTTCTAACTTGTCATTCCTGGTAGAACAAGCTTTATTTTTC
GAGCCTAGCAATGATCTACAAGCAGATGTTATCTCAGTGCCTTTTGCAATTTGTTGTGTGGGTTTTTTTTTTTTTAAAGCCACACAATAATTTT
GGAAAACAATGTATGGGTAGAACATGTGTCTGTTAATTGCACACAAAACCACTTTTAATGGGTACAGAGTTAAATTTGAAGGAATAAGTTCTAG
CTGAAGTATTATGAACTCCAAATAATGCTTTCAGGACCTCCAAAGGTAAAAGTACTAATCCCTTTGGCCATTTATTGAGAGAGAGAGAGAGAGA
GAGTAGCGTGACTATAGTTAATGTATTGAATGTTCTTGCTACAAATAAATGATATTTGAGCTGATCGCTGTGCTAATTACACTGATTTCATCAA
TACCCATTGTATGTGAAACAGTACATACACCATATTTACAATTATGTATTTAACATTTAAAATTTCTAATATAAGTATCTCTCAAACTGTGGAT
TAACTTCTTGATTTATATTTAAATATGAATCTTAAGCAAAACAGTGAAAATAACCATCTTGATTTAGTGTTTTTCTCCCATATGTGAATTGTAT
ATACTTAGGTGAAGACAATAAAATCAACTGAACTGTAAGCTTAGAATAGGACTGACGTAATTCTGCACAGCAACTTTACTAATGGTACATTGTT
GCTTCAAAACTCTCTCTCTCTCTCTCTGTCTGTCTCAATAAATCGCCAAACGGATTAGTAGTTTACCTGTGGAGGTCCTCCAAGCATTATTTGG
AGTTGATAATACTTCAGCTACAACCAACCACAATCTCTTTTTTTTGGAGGTCCTCGAAGCATTATTTGGAGTTGATAATACTTCAGCTTCAATT
TGGAGTTGATAATATTTCAGCTAGAACCTAGTAGAATCTGTTTTTTTCCTTTGGAGGTCCTCAAAGCATTATTGGAGTTCATAATACTGAAGCT
AGAACCAAGCAGAATCTGTTTTTTTCTGAGGAGTATCGGTAGCATAAATGTGATTATAAACATAGTACACTTGATATATGGAGGCAGTGACAGC
TATTTTTACAAAATTTAAATCTGCAAATGGATTCAACATCTTTATGGGTTATTAAAATGGCCTGATTTCTTAGGTTCTTTATAGTACACGTGTT
GAAAATATATGATTAAGAATTGTTTCAAGAATGCAATTATTTGATCTTAAATTTTTATGACTTGTTAAAATAGAAATTATTTGAATATCATATA
TTTGGGTAACAAAAGGCACAAGTCTGAATGTGTTTCTTTTTCTGGAATGGCCATGCCTGCCCACTCTAGAAATACAAATATCACTTGGGCAGCT
TGAAGCAGTGGAGCCTCAATGAGACCACCTGAAGAATGTCGTCCCAGTTAGTAGAGTAAGAAAGGCTGGTTCTCCCTTTTTTTTTTGCGTCCCT
CGCCAGCCTGGCCTAAGTGTTCGTGATTGGTCTATAAGGGAGGTACCTGGCACAGGGAGCCGAGTTTGGGTTAGCCACCGTACCACTTGTCAGC
GTGAAAAGTAAGATTGTACTGGCCTGTTTAGTTTTCGGCCTCTTCTTTGAAAGTTCCACCAAGCTGGGAACCTCTTGATTGTGAGGCACAAATG
TAAGTACATCAGAAAAAAACAAAAAAACTGGCTTTAAAGCAGGAGCTTGTGGGCCCCTAACCCAGACGGGGACTAGCTTTTGGCATTATATAAT
TAAGATTTTTTAAATCCTTAATAAGCGTTTTATTTTATTTTTATTTATTTTTTGAGACGGAGTCTTGCTCTGTGGCTCAGGCTGGAGTACAGTG
GTGCAATCTTGGCTCACTGCAACCTCTGCCTCCTGGCTGTGTTCAAGTGGTTCTGCTTCAGCCTCCCAAGTAGCTGGGGTTACAGCACCCTCTC
ACCACGCCCCGCTAATTTTTGTATTTCTAGCAGAGATGAAGTTTCACTATCTTGGCCAGGCTGGGCTCAAACTCCTGACCTCAAGTGATCTGCC
CGCCTTGGCCCCCCAAAGTGCTGTGATTACAGGCGTGAGCCGCCACGCCCAGCCTAATAAGGGTTTTAAAGATAATTAGTGTGTAGGTCTGTAG
GCTTATGATGGTAACCACAAGTTGTTAATGGCATTGTCAAAAGTTTTTAGTTGCGCTTTATGGGTGGATCCTCAATTACATTTTGATTTGATAC
TTATAAAAAGAAAAACTATTTCTTCAGCTTAAAAAATTGTTTAAAACTTTGTGATCATATTGTCTACCATGTAGCCAGCTTTCAATTATATGTA
AGAGGGACTTTTTGACATTTACAAATAATACTTTGAGGTAGATATCTGAAAGCACCAGCACTTGGAAGGTGTTCAGAAGTAACAAATTATAAAA
TGAGCTAACAAACGAAAGGCAAAATAAAACCGTAAAGCAAGCAGATGGGAGGCGTGTTCAGTAACTTATTCATAATGCATCTGAAATGATTGCT
GTACTCAAATATTTAACGTTACAGTAATAGTATTTTGAATGAAAACCATAGTTGATTGTCTTTAGAGAGAGGAGTGGTACTGAAACATACTTGA
ATTCGTATTGCTTTCCTTTAAAGCTCTCCTTTAAAGACTTTAAAAAAAATTCGATAGGCATTTTCATGTTTCAGTCCTACAATCCCACATCTCT
GCTGCCTTCAGAATGCTACAACTTAGATGTCCAGCCAACAGCTTAAAGCCAATTTATAAAAATAGCATCTTTCCTAATTCCAAAATTTCACCTT
TTGTTTGTTTTGTTTTGAGATGCGGTCTTGCTCTGTCGCCCAGGCTGGAATGCAGTGCTGCAAACATGACTCACTACAGCCTCAACCTCCTGGG
CTCAAGCAATCCTCCCACCTCAGCCTCCCAAGTAGCTGGGGACCACAGGTGCAACCCACCAGGTCCAGCTAACCTTCCCCCCACCTCCCCCGAC
CCCGCTGCCACCCGCCCTGAGACAGAGTCTTGCTCTTTCACCCAGACTGGAGTGCAGTGGTGCAATCTCGGCTCATTGCAACCTCCACCTCCCA
GGTTCAAGCAATTCTGCCTCAGCCTCCTGAGTAGCTGGAATTATAGGTCCTCGCCAACCACACCTGGCTAATTTTTTTTGTATTTTTAGTAGAG
ATGGGGTTTCAGCATGTTGTCCAGGCTGGTCTCGAACTCCTGACCTCATGATCCGCCCACCTCGGCCTCCCAAAGTACTGGGATTACAGGCGTG
AGCCACCGTGCCCGGCCTAATTTTTGTATTTTTTGTAGAGACAGTTTCACCATGTTGCCCAGGCTGGTCTCAAACTCCTGGGGTCAAGTGATCT
GCCCGTCTTGGCTTCCCAAAGTGCTGAGATTACAGGTGTAAGCCACCACGCCAAGCCCAAAATTTCACCTTTTAAATTTATTCCATTTATTTTG
TAGGTGAATTTCATTTATTTCTCAGGGTTAAATTTTAGCGCTTTTGCCTGTGTCTATTTTTGGTTCACACCTCCCCAGCCCACTGTCCTGTATT
AAGTACGCCTTCCTTCAAGATCACACTAATATCTCCCTAAATGGATTTCACTACGTTTGGTCCTATTTTTGGTTCAGTTATAGTTTACAGATAC
CATCATGTTGCTTCCCTGATTTAAATATTGGCCGAAAAACTTGCTTTTTGGATTTTATCTCTCATGTTCTACTGGATTATTTTAGACTTTTAGA
ACACTATGAAATGGGTGCTTGCTTTTCACTTTTCAAACCTTTTTTGCTTAAACAAAGTTCATAAGCTTATTGTAGTGGGAAGTTCAAAGTCACA
GATCTCGTACTTACTCGCCTACTGACCTTAGAGACAATAAATCTATAGAGCCTAGTATGGGGTCTGGCAGGTAGTAAGCACTTTTAATGAATGG
AAGTTTAATATTCTGTTTGCATCCACTCGACCATAATCACCCCAAATCCAGCTTAGGTAGCCTTGAATTAACCCCTACTCCACCTTGAAGTTTT
TTATTTGTTTTTTGCTGAGACAGGGTCTCATTCTGTCACCCAGGCTGGAGTATAGTGGCGCAAACATGACTCACTGCAGCCTCCTGAGTAGCTG
GGAACACAGGCCTGTTGCCACCATGCCTGGCTAATATATATATTATATATAATATACATATATTACATATATATATATAATACATATGTGTTTA
TGTATGTATGTGTGTGTACATTATATATATATATATACTTTTTTTAGAGGCAGAGTCCTGCTATGTTGCCCAGGCTGGCCTTGAACTCCTGGGC
TCAAGCAATCCTCGTGTCTTGACCTTGACCTCTCAAAATGCTGGGATTACAGGTGCGAGCCACTGCATCTGGTGAATTAAGCCTTTTTTATACA
CTCAACTCTGAGGTATTCTGTTCATACCAATATGTTCCTTTGTATCTTCTTGCCATAGCAGGTTGTAACATCCTTTTGTATCTCCTTTCTTTTT
TTTTTTTTTTTTTTGAGACGGAGTCCCGCTGTTTAGCCCAGGCCGGATTGCAGTGGCGCAATCTCGGCTCACTGCAAGCTCCGCCTCCCAGGTT
CACGCCATTCTCCTCCCTCAGCCTCCCGAGTACCTGGGACTACAGGCGCCCGCCACCCCGCCTGGCTAATTTTTTGTATTTTTAGTAGAGACGG
GGTTTCACCGTGTTAGCCAAGATGGTCTCGATCTCCTGACCTTGTGATCCGCCCGCCTCGGCCTCCCAAAGTGCTGCGATTACAGGCGTGAGCC
ACCGCGCCCAGCCGTATCTCCTTTCAAATGTCAAATAAATACATTGTGTATACAGTAGTAAAGGATGTTGATTGGCATTGAAAGCAACTATCAA
GTTCTTGTTGATTTAGAAGACAAAAAGACAATTTTAAAATAACATCCTGGGCCAGGCTCTGTGACTCACGCCTGTAATCTCAACACTTTGGGAG
GCCAAGGAAGGAGGATCACTTGAGGCCAGGAGTTTGAGATCAGCCTGGGCAACATAATGACACCCTGTATCCAAAAAATTGTGGTGGCATGCAT
GCGACACTGAGGTGGGAGCATCACTTGAGCCCAGGAGTCCAATGTTACAATGAGCTATGATTGCGCCACTACACTCTGCTTAGGAGACAGATGG
AGACTTGTCTCTTAAAAAAATAAAAAATAACAGTATGTTCATTAAACATTAATCTCACTGATTGTGATTTTTTGTTTGTTTTTTTGAGATGGCG
TCTCGCTCTGTTGCCCAGGGTGGAGTGCAATGGCGTGATCTTGGCTCACTGCAGCCTCCACCTCCTGGGTTCAAGTGATTCTCCTGCTTTAGCC
TCCCAAGTAGCTGTGTTATTTCAGCTAATTTTTTCATTTTTAGTGGAGATGGGGTTTTGCCATGTTGGCCAGGCTGGTCTCGAATTCCTGACCT
CATGTGGTCCACCTGCCTCGGCCTCCCAAAGTGCTGGGATTACAGGCATTGAGCCACTGTGCCTGGCCTGATTATGATTTTTTAAAGTCCCCCC
CCAAATTATTGTTTGTGCAATAAATGGCAAAAAAGAAGCATTCTAAGAAAAAAGTAGTCTTAAAAGTTGATATGGTGTCAGAATTGTAAGAGAG
AGGGTCTTGGCTGAAAAGCCTTTTTAGAAAAAATTGCTCCCTACAAAAAAGGCCTTTTCTACCTTTTCTCACATTTGTTTCTTTTTATAGGACG
CATCTGAGAAGACCAAATCTCTTCAAAGTTAAGCTTTTTGTTTTTTTGGGGGGGACGAAGTCTCGTTCTGTCACCCAGGCTGGAGTGTAGTGGC
ATGATCTCGGCTCACTGCAACCTCTGCCTCCTGGGTTCAAGCAATTCTCCTGCCTCAGCCTCCCGAGTAGCTGGAAATTCAGGCGCCTGCCACC
ATGCCCAGCTAATTTTTGTATTTTTAATAGAGGCACGGTTTCACTATATTGGCCAGGCTGGTCTCGAACTCCTGACCTCATGATCCGCCTGCCT
CTGCCTCCCAAAGTGTTGGCATTACAGGCATGAGCCACCGCACCCAGCCCAAAGTTAAGCATTTTTTAACCACAAGGTGGGAGGTGGAAGTATC
TCGTTGGAAGACTAAAGGTGTCCACTAACGGTAGACCCTTTTTTTTTTTTTTTTGAGATGGAGTCTTACTCTGTTGCCCAGGCTGGAGTGCAAT
GGCGCGATCTCAGCTCACTGCAAGCTCCGCCTCCCAGGTTCAAGTGATTCTTCTGCCTCAGCCTCCTGAGTAGCTGCCATTACAGGTGCATGCC
ACCATGCCTGGCTATTTTTCATATTTTTACTAGAGATATGGTTTTGCCATGTTGGCCAGGCTCCTCTTGAACTCCTGATTTCAGGTGATCTATG
TGTCTTGGCCTCCCAAAGTGCTGGGATTACAGGCATGAGCCATTGCACCCGGCCTCAAAATTATTTCTAATGTGTGCAAAGATATCTGATAAAA
ACTACATGACTNNNNNNNNNNNNNNNNNNNNGCTGGAGTGTAGTGGCATGATCTCGGCTCACTGCAACCTTTGCCTCCTGGGTTCAAGCAATTC
TCCTGCCTCAGCCTCCCGACTAGCTGGAAATTCACCCCCCTGCCACCATCCCCACCTAATTTTTGTATTTTTAATAGAGGCAGGGTTTCACTAT
ATTGGCCAGGCTGGTCTCGAACTCCTGACCTCATGATCCGCCTGCCTCTGCCTCCCAAAGTGTTGGGATTACAGGCATGAGCCACCGCACCCAG
CCCAAAGTTAAGCATTTTTTAACCACAAGGTGGGAGGTGGAAGTATCTCGTTGGAAGACTAAAGGTGTCCACTAACGGTAGACCCTTTTTTTTT
TTTTTTTGACATGGAGTTTCACTGTCCCCCAGACTAGACTGAAGTGGCGCGATCTCGGCTCACTGCAACCTCTGCCCCCCAGGTTCAAGTGATT
CTCCTGCCTCAGCCTCTAGAGTAGCTAGGATTACAGGGGTCCACCACCACACCTGGCTAGTTTTTGTATTTTCAGTAGAGACGGGGTTTCACCA
TGTTCGCCAGGCTGGTCTCGAACTCCTGACCTCAGGTGACCCACCTGCTTTGCCCTCCCAAAGTGCTGGGATTACAGGTGTGACCCACTGCGCC
TGGCCGTTTTTTTTTTTGTTGTTGTTGTTGTTTGTTCGTTTGTTTGTTTTGAGACAGTCTCTCACTCCAGGTTCGAGTGCAGTGGCACTATCTC
AGCTCACTGCAAACTCTGCCTCCCAAGGTCAAATGATTCTCCTCCCTCAGCCTCCCAGTAGTTGGAATTACAGGCGCATGCCACCATGCATGGC
TAATTTTTTGTATTTTTAGTAGAGACGGGGTTTCACCATGCTAGCCTTGAACTCCTGACCTTGTGATCTGCCTGCCTCGGCCTCCCAAAGTGCT
GGGATTACAGACATGAGCCACCATGCTAGCCTTGAACTCCTGACCTCGTGATCTGCCTGCCTCGGCCTCCCA
HUMAN SEQUENCE—mRNA
GCACCGCGCGAGCTTGGCTGCTTCTGGGGCCTGTGTGGCCCTGTGTGTCGGAAAGATGCAGCAAGAAGCCGAGCCCGAGCGGCGGCCGCGACCC
CTCTGACCGAGATCCTGCTGCTTTCGCAGCCACCAGCACCGTCCCTCCCCGGATTAGTCCGTACGAGCGCCCAGTGCCCTGGCCCCCACAGTGG
AATGATCCCCGAGGCCCAGGGCGTCGTGCTTCCGCAGTAGTCAGTCCCCGTGAAGGAAACTGGGGAGTCTTGAGGGACCCCCGACTCCAAGCGC
CAAAACCCCGGATGGTGAGGAGCAGGCAAATGTGCAATACCAACATGTCTGTACCTACTGATGGTGCTCTAACCACCTCACAGATTCCAGCTTC
GGAACAAGAGACCCTGGTTACACCAAAGCCATTGCTTTTGAAGTTATTAAAGTCTCTTGCTGCACAAAAAGACACTTATACTATGAAAGAGGTT
CTTTTTTATCTTGGCCAGTATATTATGACTAAACCATTATATGATGAGAAGCAACAACATATTGTATATTGTTCAAATGATCTTCTAGGAGATT
TGTTTCGCGTGCCAAGCTTCTCTGTGAAAGAGCACAGGAAAATATATACCATGATCTACAGGAACTTGGTAGTACTCAATCAGCAGGAATCATC
GCACTCAGGTACATCTGTCAGTGAGAACAGGTGTCACCTTGAAGGTGGGAGTCATCAAAAGGACCTTGTACAAGAGCTTCAGGAACAGAAACCT
TCATCTTCACATTTGGTTTCTAGACCATCTACCTCATCTAGAAGGAGAGCAATTAGTGAGACAGAAGAAAATTCAGATGAATTATCTGGTGAAC
GACAAAGAAAACGCCACAAATCTGATACTATTTCCCTTTCCTTTGATGAAAGCCTCGCTCTGTCTGTAATAAGGGAGATATGTTGTCAAAGAAG
CACTACCAGTGAATCTACAGGGACGCCATCGAATCCGGATCTTGATCCTGGTGTAAGTGAACATTCAGGTGATTGCTTGGATCAGGATTCAGTT
TCAGATCAGTTTAGTGTAGAATTTGAAGTTGAATCTCTCGACTCAGAAGATTATAGCCTTAGTGAAGAAGGACAAGAACTCTCAGATGAAGATG
ATGAGGTATATCAAGTTACTGTGTATCAGGCAGGGGAGAGTGATACAGATTCATTTGAAGAAGATCCTGAAATTTCCTTACCTGACTATTGGAA
ATGCACTTCATGCAATGAAATGAATCCCCCCCTTCCATCACATTGCAACAGATGTTGGGCCCTTCGTGACAATTGGCTTCCTGAACATAAAGGG
AAAGATAAAGGGGAAATCTCTGAGAAAGCCAAACTGGAAAACTCAACACAAGCTGAAGAGGGCTTTGATGTTCCTGATTGTAAAAAAACTATAG
TGAATGATTCCAGAGAGTCATGTGTTGAGGAAAATGATGATAAAATTACACAACCTTCACAATCACAAGAAAGTGAAGACTATTCTCAGCCATC
AACTTCTAGTAGCATTATTTATAGCAGCCAAGAAGATGTGAAAGAGTTTGAAAGGGAAGAAACCCAAGACAAAGAAGAGAGTGTGGAATCTAGT
TTGCCCCTTAATGCCATTGAACCTTGTGTGATTTGTCAAGGTCGACCTAAAAATGGTTGCATTGTCCATGGCAAAACAGGACATCTTATGGCCT
GCTTTACATGTGCAAAGAAGCTAAAGAAAAGGAATAAGCCCTGCCCAGTATGTAGACAACCAATTCAAATGATTGTGCTAACTTATTTCCCCTA
GTTGACCTGTCTATAAGAGAATTATATATTTCTAACTATATAACCCTAGGAATTTAGACAACCTGAAATTTATTCACATATATCAAAGTGAGAA
AATGCCTCAATTCACATAGATTTCTTCTCTTTAGTATAATTCACCTACTTTGGTAGTGGAATAGTGAATACTTACTATAATTTGACTTGAATAT
GTAGCTCATCCTTTACACCAACTCCTAATTTTAAATAATTTCTACTCTGTCTTAAATGAGAAGTACTTGGTTTTTTTTTTCTTAAATATGTATA
TGACATTTAAATGTAACTTATTATTTTTTTTGAGACCGAGTCTTGCTCTGTTACCCAGGCTGGAGTGCAGTGGGTGATCTTGGCTCACTGCAAG
CTCTGCCCTCCCCGGGTTCGCACCATTCTCCTGCCTCAGCCTCCCAATTAGCTTGGCCTACAGTCATCTGCCACCACACCTGGCTAATTTTTTG
TACTTTTAGTAGAGACAGGGTTTCACCGTGTTAGCCAGGATGGTCTCGATCTCCTGACCTCGTGATCCGCCCACCTCGGCCTCCCAAAGTGCTG
GGATTACAGGCATGAGCCACCG
HUMAN SEQUENCE—CODING
ATGTGCAATACCAACATGTCTGTACCTACTGATGGTGCTGTAACCACCTCACAGATTCCAGCTTCGGAACAAGAGACCCTGGTTAGACCAAAGC
CATTGCTTTTGAAGTTATTAAAGTCTGTTGGTGCACAAAAAGACACTTATACTATGAAAGAGGTTCTTTTTTATCTTGGCCAGTATATTATGAC
TAAACGATTATATGATGAGAAGCAACAACATATTGTATATTGTTCAAATGATCTTCTAGGAGATTTGTTTCGCGTGCCAAGCTTCTCTGTGAAA
GAGCACAGGAAAATATATACCATGATCTACAGGAACTTGGTAGTAGTCAATCAGCAGGAATCATCGGACTCAGGTACATCTGTGAGTGAGAACA
GGTGTCACCTTGAAGGTGGGAGTGATCAAAAGGACCTTGTACAAGAGCTTCAGGAAGAGAAACCTTCATCTTCACATTTGGTTTCTAGACCATC
TACCTCATCTAGAAGGAGAGCAATTAGTGAGACAGAAGAAAATTCAGATGAATTATCTGGTGAACGACAAAGAAAACGCCACAAATCTGATAGT
ATTTCCCTTTCCTTTGATGAAAGCCTGGCTCTGTGTGTAATAAGGGAGATATGTTGTGAAAGAAGCAGTAGCAGTGAATCTACAGGGACGCCAT
CGAATCCGGATCTTGATGCTGGTGTAAGTGAACATTCAGGTGATTGGTTGGATCAGGATTCAGTTTCAGATCAGTTTAGTGTAGAATTTGAAGT
TGAATCTCTCGACTCAGAAGATTATAGCCTTAGTGAAGAAGGACAAGAACTCTCACATGAAGATGATGAGGTATATCAAGTTACTGTGTATCAG
GCAGGGGAGAGTGATACAGATTCATTTGAAGAAGATCCTGAAATTTCCTTAGCTGACTATTGGAAATGCACTTCATGCAATGAAATGAATCCCC
CCCTTCCATCACATTGCAACAGATGTTGGGCCCTTCGTGAGAATTGGCTTCCTGAAGATAAAGGGAAAGATAAAGGGGAAATCTCTGAGAAAGC
CAAACTGGAAAACTCAACACAAGCTGAAGAGGGCTTTGATGTTCCTGATTGTAAAAAAACTATAGTGAATGATTCCAGAGAGTCATGTGTTGAG
GAAAATGATGATAAAATTACACAAGCTTCACAATCACAAGAAAGTGAAGACTATTCTCAGCCATCAACTTCTAGTAGCATTATTTATAGCAGCC
AAGAAGATGTGAAAGAGTTTGAAAGGGAAGAAACCCAAGACAAAGAAGAGAGTGTGGAATCTAGTTTGCCCCTTAATGCCATTGAACCTTGTGT
GATTTGTCAAGGTCGACCTAAAAATGGTTGCATTGTCCATGGCAAAACAGGACATCTTATGGCCTGCTTTACATGTGCAAAGAAGCTAAAGAAA
AGGAATAAGCCCTGCCCAGTATGTAGACAACCAATTCAAATGATTGTGCTAACTTATTTCCCCTAG

TABLE 30

MOUSE NOMENCLATURE
ICSGNM Prlr
Celera mCG18319
HUMAN NOMENCLATURE
HGNC PRLR
Calera hCG37101
MOUSE SEQUENCE—GENOMIC
TCAGAATTTGACTGTCCTAAAGAACCTTCTTCCGCTGTGCGAGTCTGGGCAGGCAGAACATTCAGGAGAAACCACAGGGAAACAGGTCTGTTTT
TAAAATTTCCTTTTCATGCTCTGTTTCCCTTTTACCTCCTCTCCTGTTCTTTCCATATCTACTGTCATTTTGCTACCCAGAAAACCTAGTGATT
CCTCCCAGCTGACAGGGCTGAGCTTGTGTTTCCACGGAGTCCAGGTTCATGGCAGGCCTGTTGAATATGGCCCTGGAATGACGTGCTTTCCTGC
TCTTTATTTATCAGAGCCTATGACTCATAGGGCCTAGTGGACTACGCTTCCAAAAAGCATTAGTTTTTGTTATTTTCCAGTTAGCATACAAACT
GTTTCAGAATGACAGTCTCAGACAGACAGCCAAACAGACAGACAGAGAGATAATTTTACTTTGTTTAATTTTACCCTTGTCCCTCTTCACCCTT
CCTGCTAATCTTCCTTCACACAAACAATTCCTCTTCTGTTTCCATGTCATATAGATATGTACACTCCCCCCACACACACATACCCCACACACAC
TCACACAGACCCCTTTTACTTTCATATCAACCATTCTTTTCTATTGGGGAAGGTAGGGTAGGTATGCCCCACGGTGAGCACATAGAAGTCAGGA
GATCACTCTCCAGAGTTCTCTCCTGCCACCCTGTGGGATCCAGGGTTCAAACCTAGGTCATCAGGCCTGGACCAAAGGGCCTCTTACCACTGAC
CCATCTCACCAGCCTTATTCAGGTTTTTTCCCCTTCAATCTGCATATGATAGAAAACTTGTAGTATTTCTTGTTCAAAATCTGGCTTCTTTGAT
TTATTTGCCTCTCTAGTTCCATCTAGCATACAAACTATTTCAGAATGACACTCTTAGGTAGATAGATAGATAGATAGATAGATAGATAGATAGA
TAGATAGATGCATACATACATACATACATACATACATACATACATAGATACAGATAGACAGAGACATTTCATTTTCTACACTTAAATACATCTC
TGCTATGTCTATGCACCACATTTGCTCTATTCAGTCATCTGCTAATAGACACCCATGCTGGTTCCATATCCCTACTACTATAAATAGTGCTACA
TGAACATGAATGTACAAGTCATCTGTCTAAAATGCTGCCTCAGATTGCTTCAGGTATATACTTAGGAATGGTACAGTTTGGTCATATAGTCATT
CTTTTTTGTTTCTGATGAACCTCCATGCTAACTTCTATATGGCTACTCTGGTTACATACTCTACAGTAGCGTATGAACATCTGTTCCCCTACAT
ATCTGCTCCAGAACTTATTGGATTTTTTTTTGAGGTTGTTTTCAGCCATTGTGTATACAGTGGGATGGAATCTCAGTTCTGTTTGTTGGTTGGT
CTGTTGTTTAGAGACAGGGTCTTCCATAGCTTAGTGTACAGTATGTAGACTGTGGACCAGGCTAGTCTCAAACTCTCCCTGATGTTCCTGCTTA
TTTTTCCTAATTGTTATGATCACACATGTATGCCAACAAACCTGGCTTCATTGCAGCTTTCTGAAGTTTTATTTTTAGATTTATTTGTTTTATT
TTTATATGTTTGCCTATATGTGTGTACATGTACATGTGTGGGCCACATATGTGTGTGGTCCTTGCAGAGGTCAGAAGAGGGTGTCAGATCTCCT
GGAACTGGAGTTACAGATGGTTGTGAACCATATCTGAAAACTATACCCGAATCTTCCACAAGAGTATGTATGCTCATAACTGCTAAGACACCTC
CACAACCTCAATGTAGCTTCAATTTACATTTTCCTATGGCTAATGTTCCTATGAACAATTTTATATTTTATATGTATTTATTGGCTAATTCACT
TCTTTTGAGAACTGCCTTTTTTAATTTATCTGTCCATTCATTGATTGACTTTGTCTTTTGTAATTTTGAGTCAGGGTTAAAGTTTTTGAATTCT
TTCTATATTCTAGATATATAAAGATTAATAGTTGGCAGGAATTTTTCTCCCAAAGATCCTGAACGTTAATGCTTTCCCTCAAGGTTCACTTTAG
CTGTCTTGCTTAAAGCTTGATAAAAAAACTAAAGTCCTTTCCAATCAGACACGTGTGAGCAAGTTTTCCCTGAGCCACAAGCTGCAGTGCAGGA
AACTATCTGGATCAAAAACGTGTTCTCGTGATTTCCATTAAGGGATTGTCTCTTGTTCCCTGTCCCCTAAGCGAGTGCTGATAACAGTCCTCCA
TACCTTAGCACGTAAGTCAATCATTAACAATGGGCTGTACTTTTCACTGGGTTGTAAAGAATAAGAAGTCAGAGCTACAGAAATCTCCTTTCTT
GAGGTAGGCAGTGGAAGGAAGAGTGTCACTTGCCTGTAAACCTCATAACCAATATGTAGCAGGCTGCAGCCCAACACGCAGGGGACGTGTCCGA
ACAGAGGAGAGTCACAGGTAAGGAATACGGGTCCATTTCTGTTTCTCTATCCATTCTGATTCTCTCTTCTCACCTTTCCAATCTGTGGACTAAA
AATGAACAGGGTCAAGGTGTGATTACAGTATCAGTGGTAGAAAGAGGTTTCTATGGACTGACCTGTGTTTTAACAAGAGATGGGCATTAGATGT
TAGTTAATTCACAATCTCCGTCTGACTCCTTCAAGACTGATTTCTTACAAACATCTGACTTCTTCAAGACTCGGAGTTCCAGAGAGAAAATGTA
TGTCAGTTACAGGAGCAAAGATCAGCGCAACAGCAGTGGTCACATGACAGGCCAGTCCTTGGGGAGGATAAAGATGAATTAGTGTGGACTTGGG
AACATCTCCAGATCCTACTCAAAGGCAAAAGGAGGTTAGATGGTAAATTTGAAAGAATTGCTACAAACTGCGCCAAAGGAAAGGAATTATGCTT
ATTTCTCTTTTTTTGACAGACTTTGGGGGTTTTTTGGTGTTTTGTTTGGTTTGGTTTGGTTTGGTTTGGTTTGGTTTGGTTTGGTTTGGTTGGT
TGTTTTTTTGCAATGATTTGTTTTAAAATAAATTTGTCTTCTTTAGAAAGCTTCCAGATACTCACTTTCCTTTCTCAACGACTTTCTTCTGCTA
TTACATGTGGCATTCTTGTGACACATTTATGACGACGAACCAACGTTAGCATGCTATCTACTTAAGCTCTTACACTATCTACATTAACATTGAG
TATTTGTCTCAGATATTTTTATGGGTTTTAAGAAATGCATAATGACACATCACTGACTTGTACAAAATAGTCCCATGATCCTAAAAGTCCCATG
TTCTACCTAGTTCTCTTTCCCCACTCCCCTACCAAGCCCCTCAAAACCACTCACCATTTTTCCTGACTCCATGGCTTTACCCGGTCTAAAGGGT
CGCCTGGTTGCAATGAAGTAGAACGTGGTCTTTTCGGATTGGTTTGAATTAGTAGTGTGCTTTTGAGTTTTTTCTGCATCTTTCTGTGGCTCAG
TAGCACATCCCTCTTTGTCGCTGAGTAACATATGACTATGTGACTAAACTACAGCAAATGTATCCAGTTGGGCAAATGTTTTAGCATGGTAGAA
AGTGGAAACGATAAAGGTGGGGGGAGTTTAGAAAGTGGCAGTACTATAAACGTGATAGAAGCAAGAAGAACACGAAGTAGAAAAACTAGCTAGA
AAGCTAAACACAGATGCCTCAGTTGAAATAAAAATTTTTTTAAAAGCTCTGGTTCTAGGGTGGCGCCAGCTCTTTTCTAAGCATATAGATTATG
CTGGGAGACACATTACCTTCAACCCAAGAGCCTACACATGGTAGGCACCCCATAACATTTTTGAAGCCCCCAAACTCCAAGACTTTTTCCAAAA
AGAGGCTTCCCCAAGACACCAGCTTATTGGCTCAGAAGCCATATCAGGGAAGCTGTAGCTGCTTGGTCAGATGAGCCTTTGCATGCCATAGGAT
AAGAAAATATTCCCTTGATAACCTTTGTTTGACCGTGGCTTATGGAAGCTATGAGTATAAGCAAAGAACTCATGTTTCAACCTAATCACAGAAG
TAGCCCAAACAATTTCCATGTAACAACAACAAAGGCTATGAGAAGATGAGCCAGCTATGCCTTCAAATGTTCACCACATTCTAGAGCAGAGAAC
AGTCATGTACCATTATCAGTAGCCCATGCAATGATGTAGGAGCCCAGTCCAAGGTGATGCAGCTTACAGTATGGCTGTGTAGTTGTCCAGGGAA
ACTGATGGGCCAGCTGGGGCTTGCAGAACCTAAAGAGACCTGCAAGTAGCAATTTGCTAACTCAGTGGGGCTACCATGCAAAAAAAGCTGATCA
CCCAGGCTAGTCAATGTATTGGTTCAAAATCATCAACTGCTTAAAACCAGGTTCTCTGCCAGAGCATAGCATTGCTATTCCTTAATGCCCAGAT
TCCTTTCTTACTGTGTGATGAAATGGGGAATTAGCCAGACATGAAGATAGGACCTTGTGATTCAGAGCTTTTCATGTCCTCTACTAAGTTAACT
TACACACTCAGCACAGGCTAAAAAATACCTGAGGATTTTCTACACAAGACAAATCTGGGAACTGCTTTGCCCAAAGGCTATTCTGTAGAGTGGA
GCTCATCCAAAGACTTACAACTTACTGCTTGTCTCGGTTTATGCTTACACAACAAGAGGGCTTGACCATCATTGTAGACAGTTTAGAATGGATG
GTGGATTTGGAGGCATCGGAATAAGCAATGTGTAGCTCTCTATCCCACTCAAAGCTCCTGGATGGGAATACTTGAAGAAGGAGATGTCTATGTG
AAGACTGATTCTGTGAGCACATCTGCCGACCTAGAGATCTTCAAGCCACAGGGCAATAGAAACATCTCAAGCAACTTCCGTGTAATGAGAAATG
AGCAGAGGAGAATCAGACTCTGATTCCAACACAGCATCAGCTGGGGAAGCAGCTGCCTCTATGCATCAGCTTCTAGCCACTTCTGCTTGTCTGG
CTTGTCTCTAGAGCATATCCTGAACATAAAGTGCCTGAGACAGGAACAAGCAGTACCTAGGCTAAAGCCACAATGACACAGAACTTACCTTGCT
TGTGCAAAGCACCAGACTCACTTTCAGAACCTGGGGCAAGTCTAGATCCCCAATTTTGAAAAGCATTTTGATGATTTCATATATCTATAGGATG
GATTTTTTTGTCATTTTCATGTGAGCACATGTGTCTCCCCCTTCTCCTGCTGGAATTTTTCTCAACTGGTGTCCCATGTTCTCTTTTTCTGTGT
GTATGGTGCCAGATAATTGACTCCAAATTTTTCTGGGAGAAATGTGCATCTATATCTCAAGACTTTGTTTTCAAGGAACTAACCAAATAGGATG
TCCATCTTACTTCCTTGTTTATTTGTATTTTCTTTTATGTCTAGAAGAAATTAGTCATGAAAAATCTAAGACCAAGTCCAACATTTGGTAGTAG
GGGAGAGGAAGAGAAGGCAAGGTACTGTGTCCTGGGAATGTGACTGATTTTCCAGACAAACCTTCTCGTCTTCTTGTCCACATTTTCTCGTGAT
TTGTTTCAGAACCCTTCCTGACCTCACTCTTTTGACCTTATTTCATGCTCTTCTCTCCACCTGCCTGATAGTACTCAGCTGCAGACACAGGACA
CATTTATGCATCTCTCATCAGTATAGGGCTCAGGATTATCCCCCAAGGGATATGGCGTGGGTGCAAAGATGAGAGCACAAGGCAGAGAATGTCA
CGAGATGGACACAATCTCTTCCATAGAAGCCCTCATTTTTAGGGATATTAAAGGACAGACACTATGGCAGAAAATTGTACAATGTCCTTGGCAT
TATAAAGTTAAACTCTGGGCCCTTTGACCATCCATATGTTCTCTCCATGTAGGTTAATCAACCACAAATTATATAACAGGATAGGGGTTGGAAA
TACAAAGGTGGATGAAGTAGCCCTTGTTCCCAAAGAGCTAATGGGTGAGACTGGCTCATGGGTCCATGATGGTGGCAAAAAGTACGGTGACCTG
GGACCACATCACAGAAACTTGTAATGAGGTCAGCTCAAGAGGCCCTGGGATCACATTGTGATATACGATAGAGCTCCTAAGTACCAAACTGAAA
GATGAAGCTGAGAGGAAGATGTCTGCGGAGAGCATTACACTGGAAAGAGAAGCCCCAGAGAGCAGAGAACAGAAGCACAGAAGTAATACAGTGG
TAGCTGGAAGACTGCAGGTCAAATAAAGGAAATTGTGATGTTTGTCCTGGGGAGAAGTGATACCTGAAACAGAAACAGGGCTAGCCCCTCTGGA
TGGACTTAAGCCATGCAAGAAGATAGAGACTAAGGAAAACCATCAGAGCATTGCAGATAGATGAGGCATGAAGGGAGATTTGCATTTGAGGAAG
GAAAAGTCTGGATGGAATGTGAAGAATAGTTTAAAGGAGACAAAACAGAAGACTTGGAAAACACGTGGGAGGCTGTTGACATGAGGTGGCTTAG
AAACAGTGTTCCTAGGAAGTCTCGTTCTACACCTGTCACCTCAGGGTTCCTGATGACCCGTGAGCAAGACTTTCTAGAAAGAGCCGGTTCATTA
ATCACGTCACTTCTGTGGTATTAATAAAGCAACTATCATGGCTTTATTTTGTATCGACAACACTATCATGTAAATATTCCTTACTGATAAGGGC
CAGTCTCCCTCAGCATTTGTCAGCCACTAGGCTCAGGGAGCCAGCTACCTTGCATTTCTTCACTGCCTCTGTAAAGAGAAGTTTTCTCTTTGAT
TATGAGTAGATACACAACTGCCTTGATGCTTGTTCTAGCACAGCGGAAGAGGATAACTACCAGGGAAACTCGCCAATCACAAGGAAATCCGAGT
GTTTCTCTCGTACCCCAGTGTTGTCAGTTAGGTTTCATTTCAAAGTCAAGATTGTTCAAGCCAAAGTACAATTGGTTTTAAATGCTGTCTTTAA
AGAAGGAATATGTTAGAGAAAATTGCTTGACACTCATTAGTGTGAAGCATGAGGGCAGCACTTCATGGCTGTGACTCAGTCTAAAAATTTCATC
ATCTCTACTAGTTTGCCAGGGTGGCCATAGCGAAGCCTAGCCAACTGGGGGATCTCAAATGACAGAAAGATATTGCAGTTGGGAGGCCAGGAAA
CAGAAATCAAGGTGATCAAGGTAGCAAAAGTGGCTGTCTTCTGAAGTCAGTGAGGAAGAATCTGTTCCTTGTCTTTCTCTTCACTACTAATGGG
AATCGGTTGCACTCCTTGGCTCATAGAACACCCTTGCCTCTTTTTTAATGCTGACATGGTTCTCTCTCTCTACTCTCTCTCTCTCTCTCTCTCT
CTCTCTCTCTCTCTCTGTGTGTGAGGGGAGATTTATCTTTGTCAGTATGAACCCATATGTCTAGACCGAGCCATCCCATTTTAATAAGTTAGAC
TGAAGCAAAGCCCATCATAATGGGCTCCTCTCAGCTGCGTCCCTCTGCGATGACCTTTTTTCCAAATAAGATCCAATTCTGAGATGTCAAGTGT
TCTTATGTTTCAACATAAGAATTTGTTGTACATTGACAGATACAATTCAGTCCAAATACCACCTAATATGATTTCAAAATTAAATAAGTTATCT
TCTATTTAAAAAAAATAAAACCTTGAAGATCCTCCAGTTTCTGAGTTCTTCTTCTGTTTCTTTCTTGAGAGACTTGAAGTTCTTCTCATACAGA
TTTTTCACTTCTTTGGATAGAGTTACCCTAAGATATTTTATATTATTTGTGAATATTATGAAGGGTGTTGTTTCCCTAATTTCTTTCTCAGCCT
GTTTATCCTTTGCGTAGATGAAAGCTACTGGTTGGTTTGAGTTAATTTTATATCCAGCCACTTTGCTGAAGTTGTTTATCAGCTGGAGAAGCTC
TCAGGTTGAATTTCTGGAGTCGTTTATGTATACTATCATATCATCTCCAAATAGTGATACCTTGACTTCTTTGCCAATTTGTATCCACTTGATC
TCCTTTTGTTGTCTTATAGTTCTAGCTAGAACTTTGAGTACTATATTGAATAGATAGGGAGAGAGTGGGCAGCCTTGTCTTGTCCCTAGTTTTA
GTCGGATTGCTTCAAGTATCTCTCCATTTAACTTGATATTGGCTATTGGTTTGCTGTGTATTGCTTTTATTATATTTAAGTATGAACCTTGAAT
ACCTGATACTTCCACCACTTTTAACATGAAAGGGTGTTGTATTTTGTCAAATGCCTTTTCAGCATCCAATAAGATGTCGATGTGATTTTTTTTC
TTTCAGTTTATTTATATAGTGGATTGTATTAATGGATTTTCATAGTGAAACAACCTTCCATGCCTGGGATGAAGCCTACTTGATCATGGTGAAT
GTGGTTGATCGCTTTGATCTGTTCTTGGATTTGTTTTGTAAGAATTTTATTGAGTATTTTTGCCTCAATGTTCATAGGGAAATTATTCTGAAGT
TCTCTTTTTTTGGTTGGGTCTTTGTATAGTTTAGGTATTAGAGTAATTTTGGCTTCATAAAATGAATTAGGCAGTATTCTTAATGTTTCTATTT
TATGGAATAGTTTGAGGAGTGCTGGTATTAGCTCTTCTTCTGCACTAAAGCCATCTGGCCCTGGGCTCTTTTATTGGGGTGGGGGGATTTTTAA
TTACTTCTATTTACTTAGGGGATATGGGTCTCTTTAGATAATTTACCTGCTCTTGATTTAACTTTGGTTCATGATTCTAGTTTTGTTGATTCTT
TTATCTCTTTGTTTCTATTTGGTTGACTCAGCCCTGAGTTTGACTATTTCCTGTCATCAACTCCTTTGGGGTGTGTTTGCTTCTTTGTTGTTGT
TGTTGTTCTAGGACTTTCAGGTGTACTGTTATGTTGCTAGAGGAGAATCTGTCCAATTTCTTTACCAGGGCACTTAGTTTTTTTCAGCACTGCT
TTCATTGTGTCCCATAAGTTTAGGTATGCTGTGTCATCATTTTCATAGAATTCCAGGAAGTCTTTAATATCTTTCTTTATTTCTTCCCTGACCA
AGTTACCATTGAGTAGAGAGTTGTTCAGTTTCCAGGTGTATGTGGGTTTTCTGTTGTTTTTGTTGTTACTAAAGTCTATCCTTAGTCTGTGGTG
ATCTGATGGGATGCATGGGACTATTTCAATCTTCTTATAAAGGTTGAGGTTTGTTTTGTGACTAATTATATGGCCAATTTTGAAGAGGGTTCCA
TGAGGTGCTGAGAAGAAATTGTATTCTTTTGTTTTCGAGTGAAATGTTCTGTATATATCTGTTAAATCCATTTGGTTCATAACCTCTATTAATT
TCATTGTGTCTCTGTTTAGTTTCTGTTTCAGTGACCTGTCCATTAGTGAGAGTGGAGTGTTGAAGTCTCCCACTATTATTGTGAGGGGTTCAAT
GTATGTTTTGAGCTTTAGTAAAGTTTCTTTTATGAATGTGGGTGCCAGGATAGGAGAGTATGATGAGATACAACTGATTTTCAGTTGTCACTGA
GAGTTTGTAGTTTGGGTGAGCTAGGGCAAAATCTATGAAGTGAAGGCATTATGTCCTCCTGAAGAACCCAGAGAATGAGTTTTTCAACATTTGA
GCCTTGTTCCTATGGGCCTGGATAGGACCACCATAGTGAGTCTTAGAATGTCATTTCAACGTGCCCTGGATGGGATGAACACATGCTGCCTAGG
GGAGTAGCTGACGTGCATCAAAGTGTTTACTCGACCCAGATCACATCAGAATCAACCCTTGTAAAAATAAATAAGGTATGTTGATTTTTTGTGC
AGATGGTTAAACAAATCTGATGGGGCAAAATGAGTTCTAGGAATAAGGGATATCAGAGGGCAAAGCTGCTGAAGATAGCAAGAGCTGCACCTGG
ACAATTTTGGGGCATAGTCCCTGGCAGCTGTGAGTGGCTGTTCTAGGGGACTGCAGTGCCAAGAGAAGAATACTTGCCTGCCAAAGAGAGAAGC
TGACTCAAGGGGGCAAAGTCAAGCTGAGCTGAATTCTAGGGAAACGTAGAAGAGCAATCATCCAGAGAGGGGCTCTGATAGAGCTCCCTGGCCA
CTGTCTGCTCTTTTCAGAAGTCTGGACTCCCTGACCTCCAAGGCCTCCTCAAGGTATGGAGTTCTCTCCCTCTTGGATGTTTCTATCTCCTTTC
ACTGATGCTCAGGGAGACTTGTGACTTGAGCTGGAAAATACTGCTCTGGGAGGGAAGAACGGTCTGTTGTCCTAAAGCAGTGTGACCTTGGAAA
TGTAGCTAAACTTTTCTGGACTCGATGTCTTTCTTTGTTTTCTGAGATTCTTGGAGTTCTGGAAATCATGGGCTCTTCTGGGTGAAATGGATCT
TAGAAACCTAGCCCAGTTTTGTGGCTGAAGAAACTAATGCTGAATTGAGAGATTTCATTATTGTCAGGAAACTAATCTATTAAAGGCAGGGTAC
ATAAAACTTGAAGATCTTCCTTCTCACCAAGAACCAATTCCACAAAGGCCCTTGGTGTCAGAAGCCTACATTTGTTCCTCCTGAGAATCCTCTG
AAGTACAGTCTTCTACCTGCCTCAGACTTGTGCAAACTGGTTTCCCCAATGGCTTCCTGCTGCTGAATAAAAAGTCAGAACCTTACATGTCAGA
CACATTTCCTGCCACCTCCATTTACCCTCCTGCAGTTCCCTCTTTCTAACTGGCTTCAAGCTGAGTAGAAGCAAGGATCTGATTGTTGTGAGTG
GGCAGGTTCATCAGAGGGTACAGATAGACCAACAAAAAGACTTATTCCAAGCAATATCCAGTTTCCCAAAGGAAACTCCTATCCAATGGATGAA
AGATATTATTATGTAAGGATTGTTTTATGATTTGATATGAATTTAAATGATCCGTTGTGAAATAGAATGCAGGGATATGGCAGCACAGGTTGGA
TGGGAGCTAACTGGATGTTCTGCAGTGATTGGATTCTCCTGGATCAATATCCTCCTCCTCCCGCCCTCCCTGTCTTATCTTTGGCTTAGAGAGC
CAGGTGCATCCAAGTACAGGATTCCTGATCAGCATCAGCACCTCTCAAAATCTAAGAGAAAAGTTGGCTCCTCTGGGTAAACTGAAGCCTTAGC
AAGGTACTGATGCCTCCTCTGGGTGACTATGTTACACCTTCCAAAGTGATTTGGCTTTTATGTCCCAGTTAGATGGTTTGGTGTCAGCAGCATA
TTGTAGCTTACATTGAGGATAAGTTAAGTTATTGGTCCCACCCCCCACTTTTTTTTTTTTTTTTTTTTTTTGAAACAAGGTCTCTCACTGAACC
TGGACCAATTTGCCAAGAGTACCAAATCAAGCCATAAGCAGTCTCCTGTTTCCGCATCCCCAGCCCTAGGATCAAAGGCATGTGCTACTAACAC
CTGGGCTTATAGATGGATGCTGCAGATGTAGAACTCAGGTCCTTTTGCTTAGTGCAGCAGACACATTACAGACAAATCTGTCTCCCCAGCCCTC
ATTTTGTTCATTCAAATATCATCCAAATTTATCTGTGATAGTATAAATGAGTGAGTTGCCATTCCCCTTCTCACACTAAGCCTTTGAAATTCAG
GATGAATTTCATACTAACAGTGTGCCCTTCAATAATACAGATATATTTCCGATGGAGTCTGCTGCCATCCAGGATAACACATTTGTGCAGGAAT
ATGTGCTTTGAGCCCTGTGGTGTCTAAGGTGTATCTTTCAACCCAGATCTTTGACGTGTTAGAGAGCAGTGACATGGTTGGAGTCACGCATTCT
GTTTCAGGGATTCTGAGCAGAAACGAGCCAGGAAATAGTGACCGTTCCCTGGCTAACCATTCCTCCCCAGAAGCATCAATGTTCCAGTCCCAGG
CAGCTTTCCAGACTGCCCTAATCCCTTGACATGCCTCCAATGCTAGTTCCCAGCAGTAAGCATGTAAGACACCTGCCTTCCCTTGGTAGAGTTC
CAGTGTTAATTATTAACAAACAAATCCACGGTGCACCACAGCAAGATGTCACTGTAAGGCTACCAAGTTTCCTAGAACATGTCCAAAAGGACTT
TGTTCCCACTGGGTTCCTAGTCTGAGTCCAGACCAGTGTATACACAAACAATGGGGGTGGTATTTTTATTAGAAGAACTTCAAGCTACCAATCC
TGGAGAACACACTTGGAAGCTTTCTCTAGGAAAATGTCTGTTCCTATTTGACCCTCAACCAGACAGGTGTCTGGGAAGAAGAAAATCACACTAC
CTAAGAGGGAACTGCAGTAACCATCCATCACCTCTGGCATATTTATAGATAAATTCACCTATTGTATGCAGCAACTGTAATGGAGGATAAGCTT
TCAGGAGATTTGATTTACTAATGACATAGACACAAAGGAGTGGCATTCACACACCCTGGATATTAGAGTGGAGGGGATGTGGGAACTTTGTCCC
ATTCTAGAATATGCATGAGGCCTCTGAAGTACAGACAAGGATGGAGCCCAGAGTGCAAAACTAACAAGAAGAGGGGCAGATTGCTTCCTACCCA
AGGAGAGCTGGATATCCAAAGCTCACTTCTGTAAAGCCTTTCTAGGAGTTCCTTCAAATTTTGTTTATGTGAAACAAAGCTTTTCCTAATGATG
TAATGTGGCTTTGGCCTAAGTCACATCAAAGTACTGTCATCTTAAACTAGGGGTTAATTGTCGGGTAGTTACTATCTGTATGGCCCTGGCACAC
TCGACAGCTACTCCATTTCCGCAAGTGCAGAGCCCTGTGACTCACTGGGGACGGGCATGAGCGTTCTTGCCTTACTAGGAAGATTGAATAAGCA
CAGTCAAGGTGCTTAGCCATCTATTACAGCAGCACCATAAGTCCTGCCTTTGTTCAGGTAGCATCATGGCCTGAAGACAGGTCCAGGTAGGGTC
ATTCTCCCAGGCAGAGACAATATCCAGGTGTCTATGTAATGATACGGGTACCAGGATCTTGCCAAATCTGACCATCCTAGTCCACCCAGACTGT
TGGTATGGTTTTATCTTCTAACTTAAAACAAATGCTCTTTTGGAGGAAAAACTTTTAATTAGCTGATAATCTGATGATTATCAGGACTTCAATG
TCATCACCAGCACTAAAAGACCAAAGTCTTATGAGCAAGGGCATACAAACATACATGTCTTAGGACTCAGTCCATATAGTCAGATGGCCAGTTG
TTATCTAGATTTTAACACTCTTGGCTTACTTGTCTATTACAAATACTATGGAATTCAGGTCATTTGGAGACTGCCATCTTTCAGTTCAGAAAAA
TTATTTTGAAAATCCTTCAGTTCAGAAGAAATTGAGGTTCTTCAAAACCCATGTTTCTTGCTGTTTGCAGCCACTAATTATTCCATAACCCTGA
GGTTGCAACACTGTTCAGTAATGGGCTGTGAACCTAAAACAAAGTTATCATATGCTAATATGAATGTCTATGGCCAATCCCAGGCCAAGTAATC
CACTTATTCCCTGTGGCTTTTCCTCCAATATCAATGCAAGAGGAGTAGGGAACACACCTGCATCCTGAAGACCCTGTTTGCTATGGCCTGGAGC
TCCTTGGACCTGGTGAAATTAAATGAAAACAATATGATGATATCCTCTAGAGGGAGACCTGGGTCTGAAATCAGGATGCTCTCTGGTCATAGGA
GGCATGGTGTGGGCTTGTAAGCTTGAGTAGAGGACATGAAGCTTAAGACCAGGGCCTTTTATTACAAGCCTTTTATTGGTGATGTACCTCTAAC
AAGGTACATTTGGCAACTAAAAGTGTGTCCAGGGAAAAGCTGCTAAGAATCAGACTGAGGAGCCTAGAAACTCTGAGGAGAAGTTAGGGTGCTT
CCCCCAGAGAAACTGGATGCCAATCTATTGTTTGAAAGCCAGTCCTATAGAACAGGTCTTCTTTGTTCCAAATAATAAAAACAGGAGCTAGGTC
AAGGAAACTGTTGCAAGTTATTCTCTTACTTCTACACGCATCATGCATGCTGTCACACACACACACACACACACACACACACACACATATATAT
ATATATATATATATATATATATATATATATATATATATATATATATGCACACACATGCATACACATACAAATGTGTATGTGCATGCACATGTAC
AAACAAATGAATAAATAGGCAAATAATTGTAAAGAAAATAGTTGAAGTCAGAGCAGTGAATTGAATGAAGATTCCAAGACAGAGACCCTGGGTC
CTTTAAGCTGCCTGCTCTCTAAGGTGATGCTTTTCTTGGCATATGATGGAGAACAGGGGTACCAGCCATGGAATTGATGGGGCACTAAAGAGCC
TGTTAGAGAGATGCTAAGAGGCTCATTTGTGGATTTTAAGTGATGAACAAACAAAGTGGGTTTATGGTTTTGGTGTTCTGCAAACTGTGATTTT
CGACTTAGGCCATCCCACATATTAAAAATAAAACTAAAAATAAAATCAGGCTCCATGGGCTTTACCCATCCCATCTTAGCATGTCTGTTATTGT
TGTCCTTGCTCCATTCACGTTTAGCCAGGCATGTTGGTGAGCCTTTAAGTACAACTTCTGAAATTACTAGCAAGCACAATCTCACAGCAAACTC
CCTGATCCTCTGCCTCTGACAATCTTTCTGCCTCTCTTCCACAATGATCCCTGAGCCTTGGGTATAGGAGTGGTCTTGTAGATTGGGACTGGGA
TCCTCCATCCATTTGGATTGGTTGTGGTTTGCTGGGAGGGTTTCACTGTCTGTTGCATAGAAAGGTTTCTTCAATGAGGGGAAAACTCAGGGGT
GAGCACGACACTTCTCTGTGAGTGTAAGGACTCTAGTTAGGAACTATGCCAGCTTAGCAAAGTGCTGCTTGCAGTTTCTCCATGACGTCACTGG
CCCTTCCTAGTTGGCTGGGTTCCCAATACCAGGTGTGATTTTCCTCTTGTCAAGTGGGTCTTAGGTCCAATTAGAGAACTGTTGGTTACTACTA
AGGTATGTGTGCCAGTACTACTGAACCCTTTGTATTACCATGACATATTGGTTATAATGATAACCATATGTGTTATATGTGTGTAATATACATG
CAATATATATGCTATTTATGTGTAGTATATATATATATTTATGTGTATTATATATATATTTATGTGTTTCACATATATGTATGTGTGAGTGTAT
GTGTGTGTGTATGTGTGTGTGTGTTCCAGTTCATGGCTGTCATAGACCTACTGGCTGCTTCTCTCCTTTGGAAGCTTGCATGGTGCATTCTCTA
ACCATGAAATCTAATCCTCAAGGAACAGGCTTTCAGGTCAATTCCCGTTTGGGAGCCTCTGTCACTGCGTCTGAACTACTTGGTGTCTTCAGCA
TTAAGAACAGAATGCTAGGAAGGTGTGTTGTAAGTAGTTTGTGGTATTTTGTTTTGTTTTGTTTTGTTTTGTTGCTAAGCCAATGGGTCACAAT
AAGCAGAATGGTGATGTGGCTAAGAACATGGGCTGTGGAGGCTAAGGCCATAAGGTTCAAATGCCAAGTCTTCTCTTATTGGAGTCTGAGAATG
TGGGGCAGTTACATAATTTACTTATGACTGTGCTTTGGTGTGGAGATAGAAATGATAATGTTTGCCCCTGCTGTTATCACTAGGTTTTAATCAC
AACTATCTTTGACTCACAATGAACACTCAGTACATGCTGGAAATGATTCTCATAGAAGGAATATTACCTTTTATTTCTTAAGCCAATGTGAGAA
CTTAAAAGGCACCAGCTTGCAAACACAGAGACTATGTCTCTTTTGCATTGTGTTCTAATGAAAGGAAAATTAAATGCACATACATTTTTATTAT
AATTAGGACACAGTTCAAAAGTGATAAGCCCTCTTGGTCTAGGGAATGACTACTCCTATGACATAAAGGAAAATGAAATCAAGTCCAGTATAGA
TTTTTGGCCATGAAGCTCAGAGAGTAAGTAAGATTTCTGGAATTCCTATCATTCTATGTGGCTCTATGGCTTATTCCCCAAGAGAGGCAAACAA
TTAAGGCCCTGTGCAGAGGTGCTTCCACCCTGGTGTACACAGTGAAGTAGTAACAGCTGATGGACGACCTCATTAGCCCTCCCCTAAGCCTTTC
TGTCTCATCATGAGTCTCAGCTATGTGACCACGGGAAACAGGAGAGAAAAAGATACAATGGGGACCACGTGTCTCTCCTTAAGTACTGCTTATG
ATATTGAGGTTGGGGGTCTCTGAGCCATTTATTCCTCCTCTGGTTCTTTGGACCACTTGGGCATTGGACTCTACACCCCTGGCAGATCAAGGCA
CATTCTGTGGCTCTTGGGAGCCTTCCTGATCCTATATGGAATGACTTAGATCAATGGGAGGACAGCAGCAGACTCCTGCAGTCAGTCAAAGTGG
TTTCCACTCCTAGGAGGAACACACCAGTTTGTTTCTGTGTGAAATACAGAAATATGACAGCTTTGTCGCAACGCTAAGCAAACCATTGTGTGGC
AGAACAAGCTTCTTTTTCACGTCAGAGGTTGACACACTGGCCCCACAGACAACACCAACTGTTCCCTTTGGAAACAGAATGGTGGCAAGAATGA
GCAGTTATGTGGACATCATCATTCTGGATCATCTCTAAGATCAACATTATTTTAGAATGCTGACAATGATATGTTTTGTGGGCATGCTCCAGGG
GAGGATTCAGTACATGGAAAATACATGCTAATCATCCACACATAACTGAGGGAATCATAAAGAGTGGATGATGCCCTGAGGGAATGTGTATTAT
TTACAGAGATATCTCCATGTCAAGACCCAATGCATCAGATCCATGGGAAATCGGCAAAGCCTTCTCTGGTCTGAGTATGACTGTTGACAGTGTG
TGCAAGCACGGGTGTTCACTAACAATTTACCAGTTTGTGCAAAAGCTAGCAACTGCAGCTTCCTCCCTGCAGGTGTGTACTGCCTGAGATAGCT
CAACCTTCTTTGGAGACTAGCTAACCAACCCCCTGTCTCGTAGATAACAAAACCCCTTACGTTTCAGACTGCCTCAGTAACTGATCTCCTCCAT
GCAAAGAAGCATAGCCCATCCAAACGCAGCTACACGTGTCTGTGTCTGTCCTTTCTTCATTCCCTCACTGCTCAGTCAGGTCACCTCCAAGCTG
CACATGGAACACAGAACATCCCCATCACCTTGGGCCCAAGCGTCTGGCATATGCTTCCAATCTTCAAGTTTTGAGTGGAGAGAAAATGTGGGCT
GGAATCTACAAATGTGAGCTGGAAGCCATTCCTCATGATAACCCTCGCTCTTTCTTGCCTTTTTCTTTTCTGTGTTTTTTTTCTTCATTTATTG
CTTATGACACTAGCAACGCAACCTATTGGAATAAAAATGTGTGCTTCCCAGAACATTGTCTAACTTACTTCTCAAAGAAAGGGACCATTTCTGT
TGATAGTAAGACTTGGCTTGAAAGGGCCAGTTAACCCTGGCCCCAGTTTGTCTTGATCAACATCTCTAAGGCGTCCTATATTTGATCTAGCACC
TACAGAAAGAGTAGCTACGCTCAGAAGAAAAAAATATCTCTACAAAAGATTCACTTGTATGAAATATTTTATACACTAATCTAGCTGAATCGGT
CTCTTTCTGGTAGAAAATACAGTATCCAAATATATAGGGAGTAATTATGATAGGTAAGCCTGATAAAGAAGACCTCAGATCATGTGTGACCAGA
TAAGCCTAGCAATTGTCTGAAGACTTCAAACTACCCCTTAGGACTACTGAAAGGATGCCTGAAGAAAAACATCTAGAAACAAGTTTTGTGTGGA
CCAAACAAGGAGCAACAGAGAATTTTTGCACACAGGGAGTCTATGTAACTGCCCCAGTCATTTCTCCTAAATCTTTTTCTGCTAGAAAGACCTT
TGGTCTGGTCCCACTCATCATTTTAAAAAGACTTACAGGATTGAAATGATTTTAGCCTCTTCTTTCTTCCTTCTTTACAGAAATGAGTTCTGAG
ACGAGAAGACTTGGGTGGTTAAGTCTGATGCTGTTAAATATATCAGGGGTTGAAAAGGGCTTAGAAGTGTAACATTCAGCTGGGCATGGTGGCA
CACGCCTTTAATCCCAGCACTCCGGGGGGCAGAAGCAGGCAGATTTCTGAGTTCGAGGCCAGCCTGGTCTACAAAGTGAGTTCTAGGACAGCCA
GGGCTATACAGAGAAACCCTGTCTCGAAAAACCAAAAAGAACCCAAACAAACAAACAAACAAACAAAAATGTAACATTCTGATTGATGGCATGT
CCCTTGAAATGTTTCTGAGCTTTACTATGCATTAAATAACTTGAAATGAATGCAAGCATGCACAGTGCTTCTTTCTGTCTTACTGAATTTAAGG
ATGCCAAACAATTCTCTCATTTTTCCTTTGCTGGAAGGTGTGGCTCACCATTCTATTAGACAAAATTGTAGCTGACCTCTAATACCTAATTAGT
GCAAAACAATGCTACAATTATTGGGCATGACTTTGACACGTTTTGGCTGCTAATGCAACCACTCCTAGTTAAATAGATTGGCAATGAAGTTTCA
ATGCAAACTTTGTCAATGAGGCCAGCCTACTACTAGATATCCAACCATATAGACAGGTAATGGCTGAACAGATCTTCATTCCTACAAAACCTCA
AGAATTATCCATTTTGCAACTTAAGTATATGCTGTTCATTACTTTTGTACAAGCTTGCTCTCTCTCTCTCCCTCCACCCCCCCCCTCTCTCTCT
CTCTCTCTCTCTCGTGTGTGTGTGTGTGTGTGTGTGTGTTTTAATGGCTTACGAATCCACATAGGATTCTTGTACATATTCATTTTGGTTATTT
ATTCCCCCTTAATTAATCCCCCTTCATCTCCATGTCTCTCCCTGCCCTGCTGGACCTAACCCTCTTCTTTGCTATATTCTCTTACCCACTGTCC
TTCTTTTACATCTTTATTTCAATCATGTGGACCCGTTTCTCATTACCTAGGCTTTATCCATACTCACTTCTACTTAGAGACACATGTTTAACAG
TGAAAGGATAAGATTGCCATCTGAAAAAGGGTGTGGGGATATGGCGTGTTTATTTTTCTGAGTCTAATTTCTTGAGTTCTTTGTATATATTGGA
CATTAGCCCTCTATCAGATGTAGGATTGGTAACAATCTTTTCCCAATCTGTTGGTTGTCATTTTGTCCTATGACAGTGTCCTTTGCCTTACAGA
AGCTTTGCAATTTTATGAAGTCCTGTTTGTCAATTATTGATCTTAGAGCATAAGGCATTTATGTGCATGTGTTTGTGTGTATGTGTTTGGAAGC
AATGCTTCTCCCATTTCTTGGAGACTTTTTTTTTTTTTTGGAGAGCAGTTGCCTAGATGTTCATCAGCAGGTGAATAGAAAATGTGGTGCATAC
CTACAGTAGGATTTATTCAACAATAAAGATAAGCAAAATTACAAATTTTATAGGAAAATTGATGGAACATCTCTCTCTGTCTCTGTCTCTCTCT
CTCTCTCCCTGGTGGTGGGCATATAAAATGCAGTAGCCACTTCAGAAAACGGTCTGACAGACATCACTCAAGAGCCTCAGACAAAAGGTTGTCA
TGTGAGCAAGGGAGTCCACTATTGCCTATATATTTCTCTACGTTGGTAACAGATATAGTATGTAAAAGTGTTTGTAGCAGTACTATAACACTAT
GCATAGGTAAAAAACAGCTCAATGTCTATCAACAAATAAGGAAAAAAAATGTGCTAAGTAATATGATGGAATAGTACTCAACTGCAGAAAAGAA
TGAAGTAGATGACATAGTGATGCAGTGTAGAACACAGATAAGGATCAACAGTATTATGCTAGGCTAAAAGTCACTTATTTGTTGTCTATAGGAT
AGAACCTGAATAGGTAAATCCAAAGAGAGAAATGCTAGAGTAGTGTTTCCAGGGGCTGAGAGAAGACTAAACTGAGTGTAGATGGATTATATGT
CTTCTGCTGCTGCTCCTTCCATGTCTGGCTGACGACAGATGTTTTGACAATGGATGAGAAGCTGATGAATAATTTTTTTAAAAAAATACTGAAC
TTTACGTTTTAAAAGGGTAACTGTTATATGAGTACTCTCAATTTTAAAGGAAAAAAAGCAATGTTTAAAAGTGGGCCAATGTTGGAAGTCAACT
GTGAGAAGTTAAGAGCAGATTCACAAATAAGCACTGGAATTGGGCCTTGATTTGGGGGAATATTAAGAATATAACTAGAAGACGTTGTAAGAAA
AGTTTGGATGAGTCAGAAAATAGGGAGACCGGCTAGAGAGATGTCTCAGTGTTTAAGAGCACTGATTGTTCTTCCAAAGGTCCTGAGTTCAAGG
TCCCAGCAACCACATGGTGCCTCACGACCATCTGTAATGAGAACCAATGCCCTCTTCTGGTGTTTCTGAAAAGAGTGACAGTGTACTTGCATAA
AATAAATAAATAAATAAATCTATTTAAGAAAATAGGGGGACAATTGTGTGTTAGCAAAATACAGTAGAGACTAGACTAAGGTTCATTAAAACAC
AGGCCAATAAAGATGAGGCCACATGTATGTGTTCAGGACACATATACTGTAATACGTAAAATACATTAAAAAGTTCAGACTTCTTCAGGCTCGT
GAAAAACACACTGTGAATGCTTTAACCCTCATTGTCTGATCGTAACCCATTCCTAGTCATCTCCCTTTCCTTTCCAGCTAGTTTGCATCTTACG
TCCGCGTGTGTACATGTGCAGCTTCTATATAGGCCGCCACAGCTGATTCCTGTTTGTCCTTTTCCCAATTTTTTATTTGGTATTTTCTTCATTT
ACATTTCAAATACTATCCCGAAAGTCCCCTGTACCCTTCCCTCCCCCTCCTCTACCCATCTACTCCCACTTCTTGGCCCTGGTGTTCACCTGTA
CTGGGGCATATAAAGTTTGCAAGACCAAGGGGCCTCTCTTCCCAATGATGGCCAACTAGGCCATCTTCTGCTGCATATGCAGCTAGTACTTGGA
TGGAAGGCCACAACTGACTCTTAAAGACTGTATGTAACACCAAATATTAGAGCAATTGTCTTCACTTAGTAGCATTTCTGAGGGTTTAAGTCCC
TTTCATTTGGATTCTCAGTTTCCATGTGCAATGAATTTTTTTACAGGTGAGCAAGAAAAGTATGGAAGGAAGCCTAAGAAAGGCCGCAATGAGA
CTTTAAAAAATGATGCCAATTTTAGAACGCATCTTCTCTTTCAGACTTGGAAAATTAACTTTTATTACTGCCCTATGCAAACTAAATTTAAAAT
AAGCTTTGGATTATACCCCGTGCGTATGAGCGGACGTTATGACATTGATTGACAACGTCAGATCTACCCTCAACCCTCTGAGAGTATGATTCAC
ACATACCTGGGATCAGAAAAGAAGTGTTTTCCAGACTAGGCAAGTCACTAGAGCTCTGGTGGTACTCAGGGTAGATTTCTTCCCCATAATTCCT
AGCTAAAGATGAAAGATACAGACAAATGACCCATCCTAGGAGAATTTACAAGGGTAGTGGTAAGCAGGTAGGTTTCAGAACAGCTCATGAAACA
ATTCTGCAGCAGGAGTCAAGGAGTCCCAAAGAAGTGGCCTATATGGGACAGCAAGGAGAGACTCAAAAAAAAAAAAAAAGGAGGAAAAAAAAAC
CCTATGTGGGGCTCAGCAGTGAAGAGCACTGAGTGCTCTTTCAGAGATCCTGAGTTCAATTCCCAGCAACCACATGGTGGCTCACAACCATCTG
TAATGAGATCTGATGCCCTCTTCTGGTGTGTCTGAAGACAGCTACAGTATACTCATATACATAAATCTTTTAAAAAAAAAGTCCTATGTAAAAG
GCATAATGGTAGATGTTGCAAGAAAGCAGTGTGTGTATTGGTATCTGGCAGGAGGCTCAGGAATGTTCACATCACAGTCGTTAGGAAAGGAAAG
CAAGGGACCCCTTATTGTAGGATATATGGACAATTTAATCATGGGGTTTTTTTCCCCCAATAATAAAATTTTCAGCAGGAAAAAAAAATAACAG
TAAACAGTAAGCTACGTGTAACATAAGGATGAAAGATTTCAAAACCCGTCTTTTACAAAATGCACAGCAACAGCTTCTTTTGTGACAGCATCAT
CAGTCCCCGTGGTTTGCAGCCGGATGTAACAAGCCCTTTTGAAGAGCTAAGAGGGCAGAAAGTTTCTGTTGCCAAATATATAAGAGAAGCAGGA
AGCACTTAGCCTCGGACCAGGAAGGGCATCCTTGCAGGAGACCCTGCAGGTCTGTGCAGTTCATTGCCCAATGCTTCCCTTAAATAAAGAGCCA
CAGACTTCAGGAGTGTGTTCCCCCCTGAGTGATTCTGCAAGAGTTGTTTGCCCTCCAGCACTGGGCTGTGCTGAAAGAGTTTAGTTTCCCAGGG
AACAAGATCTGGGTCTAAGGCAAGGCAAGCAGCATCCTATGCCCAAACCAGTAACTCCGTTTTCTCAGCCTGCCAATCACATTGGAGCTTCCAG
ACCATCANNNNNNNNNNNNNNNNNNNNGCCTCAAGTCTCCAGTCCCCATCATGATAGTGAGACCCAGAGGAAAGGGAACACCTGTTGCCACCAT
TTTGAAGGGACAGTGGTGACCCATAAAGAGGGTGCTCTAAAGGAGACAGGACAATTCAAGGACTTGGATAAAATATCAAACTGACAGTGGTGAG
CAACTGTCAGGTTCTAGGACCAGTTTAATCCTGACTGGGCAGAGGATAGGGGCCATCCACACAACCCCTGGAGGGAGCGTTCTATAGAACCGGC
TGCCTTGGTCAGCTTAGGGCAACCAGCATTAAGGCAGCAGGAGGAGGGATATAACCCAGGCTCACCTCTCCCAACCCCTACACAGGGTTCCTTC
CTGCCAAGGCTCTCTACACCTTTAGTTTACAGTGGAAATCAGAAGGCAGTAGGACTCAGGTGCAGACCATATAATTCAGAACAGGAAACAGGAA
ATGTGGAAGAAGGAGGGGCTAAGGGAGAGCACAAACATTGGCCAGCTGCAGCCCGGCCCCAATTGGGACTGCAGACTTGGGTAGATGGATGGTT
GCCACATCCTGCTCTGAGCCTTTTGAGTGACTCTGAGATAGAGCGCCTCTGTGGAGTTCCACAGAGCAGGCACACAGGCTGAAATCATGAGGTT
TGTATTTTACCTCCTTGCCGGTTTGCAAGTCTTGAAAGCTCACTCCTCTAAGCTGATGGCAGCCCTCATGCAAAGCAATCTGATTGCTGTGTCT
GGGGTTCAATACACACACACACACACACACACACACACACACACACACTCAGGTGCACGTGCATACACACAAAGACAGAACAGTTTGCTTGCAA
CATTGTCATAGCTTAGTGAACAGTATAGCATTGTTCTGGCTCAAGAAGCCCTGGTTCTTCAAAGCTCCTACTTAGATGAAATTATTTGCATCAC
AAACAAAAATTGTTTTGCATTTTTTAGATAATGAAGGATCGTTTAAATATATAAATTAAGGGCTATTTAGAAAAAATAGGTGTCTTTGTGAGAC
TGGCTTAATTCACTTACCATGATCATATTCAGTTATGGTCATTTTAAATACAAATCATGTGTGTTCCAATGACGTGAGATTACAGTGAACTATC
TAGGGAAACTAACAGGAGTGGAGTAGGGACAGGAGAGGAAGAGAAACGATGGGTGATGGGCACATATGCTCAAAACCATAATGTACTGGGGCTG
GAGAGATAGCTCAGCGGTTAAGAGCACTGACTGCTCTTCCAGAGGTTCTGAGTTCAATTCCCAGCAAACACATGGTGGCCCACAACCATCTGTA
ATGGGATCGGATGCCCGCTTCTGGTGTGTCTGAAGACAGTGACAGTGTACTCACATACATAAAATAAATGCATCTTTTTTTAAAAAATCATTAT
ATACTTGCATGGAAATGACCCTATGTAACCGAGTTTCATTTAGTTGTTTTTCAATTGTTAGAGAAAGACTAAATTTTCTCTAAGATAGCCATAT
GAAGACAGTTGATCACTCTATGTCCCAGAAGCCCATCTGGAACTTAGCATTTTGAGCAAACCATAAATTTGATGAAATACCTCCTATTCAAGGC
GGCAAGCATTCATTTGCCTCACATTTCTAAGTCTGCCCCTGTGACTGCCTTAGCTCCTTCTTCCCAAATTCTCCAGTTACCTGGCCCTTAGGGG
TGGTAGCGAAATCTCCTTCCTGACAGCTTCCTTGGCCAGGTGCTTCTGTGTGGAGTCAGTGCCCAGGCCGCCAGCTGTTCAAGTGTTTGGGCAT
ATGCACACACAGGAACGACTTGGGCAACACCCTTTGCCTTGTTTCGGGATGTTCTTCTTGCAGACCCTTGCCCAGACATAGGGAGTGCCTGTGA
AGTGCTCCAGAATAAATCGGAATGTCCCACTGAAGCCAGACACCGTGGTCTTCATGTTAATGTGTAAGTAGAGTAGTGATCATTTATTTATTCT
TTGATTCCCTTTTGCAGAGTTTTTATTGCCCTGTCTGAGATATTGGAGTTTTCTGCTACAGTAAATTTAGCAGGATTTCTCCAAGGTGACTTTA
GCGCCAGTGAAGGACTTATTGTGCCTCTTCAAGTGGTTCTATCACTCTGCTTGTGCTAGGGGTAAAATGAGGTGCCAGGTGGATTCAGGACAAG
CAGACACAAGCAAGCTAGGAAACCTGGCACTCGATCCCCTCTGCGGTGCTTAGGCAAGCCTTCTCGGCTACTGAATTTTGAACTTTCACAAGAA
CAGATCGGCTGTTTGTTCAATGGTTTATTACTCCTGCCACAACAACGCATGGCATGAGGTTGGCGCGGCCTGTTTATTTCTTTGTGTATATGGA
TATGTGTTTGTGGGTGTACATGAATGGGTGGTATGTGTACACACGTGTGCATGGGGAGACATGCCCAGGGCTATTTAAATCTTCTGCCCTTTTC
CTTCACTTCCCTCCTGTTCCTTCTTAAGAACAAGCATTGCTTCACCTTTTCACTTAAATTTCTCCTCTAGAAAAAACTGTTATCATTCAACTCA
ACATGTTTTCCACTGAAACTTCTTCCATATTAATTTTTAGGTTTTCATGTTCTCGTTTTATTCGTACTTATTGGCCGCTATTTTCAAATTAATA
GAGTCCAATGTCATATTTCTGTATATTGCATCCCTGTTGCCCATCTATGCTTCTCCTCCAGACTATTTCTTCCTGTGCTTATTCGCTTAGTGAA
AGTACATTGCCAGGCTAGAAAGGTGGTTTGTTGGCTAACAGTGTTCTCTGCACAAGCCTGGGAACCCGAGCTTGGTTCCTCATATCCAAGAAAG
GAGAAAAGCAACTCTGAAAGTTGTCCTCTAACTTTTTCATGTGCACCGTAGCCCATGTGCACCCACACCCACAAATACACACCACACACACATA
CACAAAATAATAATAATAATAATTTTAAATATATCAACTGACTTACAAACATTTGTTTTTTTTTCCCGATGACATTTGTGATGTTAACGCATAA
TTTGATTCTAATTTAGTTGTAAGCATATTCTATTTTGACTCTTTGAAGTTTTTTGAAATTCAGTATCTAGCTGTGGTGGTGAGCTTTCCACATC
AACTTATCATAACCAAGAATCACTGGGAAAGAATATTTCAGTTGGAAAAATTATTTGGATCAGAGTGATCTGTGGTCAGGTCCACGAGAAAATG
TCTTGGTTGCTAATTGGTGTTGGGGGACTCACTGTGAGTAGCACCATTCACTCATCAGGGGACAGTGAGCTGTCCAAGGCAAGAGAAAACGAGA
AGACCTCAAGCAAGCAGGCCGCATGGCTATTTGTTCATGCCTGCTTGTGACTGTAGACATTATGTGATGAGCTGCCTTGAGAGTCTGCTTCAGT
TTCTCCTCAGTGACAGACGGTAACCTGGAATTGTAAGACAATAAACTCTTTCCTCCCATAAGCTGCTGCTTCTTGGATGCTTTCTTGCTACAAC
AGAAATTAAACCAAGGCATCAGCTTATATTCTGCCTGAGTGCGTATCCCAGGTGCCCAGAAGTGTTGGCTGTTGTACTCTGCACATTTCAATGG
AGTATCAGTCAGGTTATGATGTCCTTCCTAATTTTTTAGCAAATGTGGTTTATTTTATTTTTTTCAATTTTTATTATTAACTTTTATTAATTAT
TCCATTCATTTACCTCTCAAATGATATCCCCCTTCTTGGTTACCCCTCCCCAGCCCCATCCACCTTCTGTTTATTTTAATTAATTAATTAATTA
ATTAATTAACCAGTACAGAAAAAGTCCACTGAAAAATCCATGTCCTGGGCCCCAAAATACAAATAAAATCAAAAGGAGTAGAGTCCTGCTGCTC
ACATTTCTTCGTGAGTAGTTATAACTTCCCTGAGATTTTTTTATACCAGGGTTTGTTTCCTCCTGGTTTCTCCTCAGTGACGTCATCCTGAGGT
TAGTGGTTACTGTCACTGTCTCTATCCTCATCTTGACTCCATAATATTCTTCCTCTTTCGATCCAGACCCTCAGGGTTTAAGAGTAGCTTCAAG
CTGAGAAAAGGTCAATTTTCTCCAAAAAAGCTTCCATCTCCCACTGAAAATGGCCATCTCAGAGTGAAGTACACTCATTCTGAAATGTCAGGGC
CAATTGGATGGTCATCAGAAAGCTTGGCCTGGTTGATATCTTTATCAGCACAAACCTGAAAATGCACAGTCCTCCAAAGGGCGGCCTCTTTGTG
CTCATCCGAAAGTGATGGAAGGAAGGCAATACAAAAGGGGCTGAACTCTGGTCACCAGAAGTCAGAAACCATGCAAACAACCAGTAATGAATAG
TGAATTCTAGAATTCAAATTAGCAGATGTTTTAAAGAGACAAGCTTTCAATTTTCATTTTAAACATGAGCAAAACCTGTGTTCTGAAAGAAAAG
ATTTAGAGAAGAGAAAAAGCAAAAAGAATATCTAACATATTGCATGACCCACCACTCCCACAATGTGCTCCTGGTAAACTTGAATCACATCTTT
TCTCCATCCCGATAATTCTCTAACTCTCAAGGAGAGGCCTGAGTTAACTCTCTGAAATTCCATATCTTAGGCACTGGATCTTCGTGATTATCAG
GATGTGCTGTCTTTATCACTTTGAAGTGAAGACAACTGCTGTCCTGATCAATGACTTTGAATTTAACTTATTCTTTTTCCGTCTTGCTCCCTAA
AGCCTTTGGTGGACAGTTTTGTTCCTTGGTTCTTGGGCTTCCCCTTCCTAAGAGTTTTGTTTGCATGTCTATTCAGATGCTAAAATATTGTGTT
AACATCCATGGTGATTATTTTAATGTTTGTTCTTTCAGTTTGGGCAGTTTGGCATTTGGGAATTATACGTACTTTGACACTATTAAATTAGATG
AATATAGAATTGTATCCTTATGATGAATAAACCTTTTATAATTACTAAACTCATGGTTTATAAATAACAAAACTCCCTGCATCTTTTGTCTTTA
TCTGATATTAGGTGTAGCTGCATCAGCTTTCTTTTGGCTAATGTTTTGATGGTGATATCTTTTTGTATTGCTTTATTTTCAGTCTCCTACTTTC
TTTTGGATTATTTAATCCATTTACAGTTAATGTACTTACTGGATTTTAAATTTTACATCTCCCTCTCACAATTTCTTTTACTTCTATAACTTAT
ATTCTTTTATATTCTTCTTGACTTTTTAAAAATGTTAATCAATGCCAATGTTTTACTATTCTCTTTGTTCTATTTACTGTCCCGTTAATACTGT
CTACTGTGATTCTCCAAGTCATTACCCAAGGATTATATGTTCTTTCTTTGACCTGTTACAGTTTACTTTAAGTTAGTCTTTCTTTTATTTTGTA
TCCACCCTGGGAATTCCTCCTGGGGAATGATGTTATGCTAGGGTACAAGCTACCATTGAGTCACCACCCTACCCTGTTAATTATAATGAATAAT
GTAACTAACTAGCTTGCAACAGCTTAATGTCATTGCAAATATTTCTCAGCTTTCCATTATTATTATTATATACTTTCATATATATATATATATA
TATATGTTACAAGATTGTTAAAGTTACAAGATATTGTTGTATTAAATGATCAATGAATGTTAAAATTTACCAAATATTTACTCTGGTTTTTTTT
TCTTATTTTATTTATGTGAGTAAATTGTAGCTGTGTTCAGACACACCTAAAGAGGATAGCAGATTTTATTACAGATGGCTGTGAGCCACCATGT
GATTACTGGGAATTGAACTGAGGACTTCTGGCACAGCAGTCAGTGCTCTTAACCACTGAGCCATCTCTCCAGCCCTACTCTGATTATTTTTATT
CCTTCTGGTGTTAGTATCATCCTTCTGTGATCATGCTTTTTACTTTTTTATTTTTCATTTAGAATCATGTTTTCTGAGTTTTGGTAATGGCACT
CCACTGTGGGAATGTGAAATTTGGGTCATTGCCATCTAAAACTCAGTCACTTTGGCAGCATGGCTCAGTTGAATGGCCTCAAATCCCAGGACAC
TTATTTCATTATGTATCTGTCTCTGCTCCAACAATTGTCCAGAGATACTGACCAGTAACAAATCTCCCAAGACAAAAAAGAAGAAAAAAACCAC
CCAGGCCATTATCAAAATCACCTCACTTCATTGTTTTTTTCCTCCAGGATCTTAACTGCTCAGATCAAATGGACCATTTAAAGAGCTAGAAATG
ATAGATAGATAGATAGATAGATAGATAGATAGATAGATAGATAGATAGATAGATGATAATAGGTAGATAGATGATAGGTATAGATAGATAAAAG
ATTAATATTTGATAGATAAATAGATAAAAGATAGGTAGGTAGGTAGAGAGGTAGATAGATGATAGGTAGGTAGGTAGGTAGGTAGATAGATAGA
TAGATAGATAGATAGATAGATGATAGGTAGGTAGGTAGAGAGATAGATAGATAGATGATAGGTAGGTAGATAGATAGGTAGATAGATAGATAGA
TAGATAGATAGATAGATAGATAGAAGTAGTTGGTGGTAGCAGTGATGATAAGAAGATAAAAAAGCAACTTTATGTTGCCTTAGAAGTAGAATTT
GTCTTCCCATATAAAACTATATACAACACTGAATCTATTATAAAACATCCTTGAACATTATGTCATGTGGACAGACAATACTCTGTATACACAA
AAGGCAATACATTCTTTATCATCTATTTAATGGTCAGTATTAGTAACCAAGTTTAATCACAACCTTCTAGATAATACTACTGAGATGCCAATGA
CTGCTTTCAATATATAAGCCTGAATTATGAACGCTCTATAATGCAGCCATTACTTTTTGAAATTTTTAATATTTTTATTGGATATTTTCTTTAT
TTATATTTCAAATGTTTTCCCCTTTCCAGGTCTCCCCTTTGGAAACTCCCTATCCTACCCTCCCTCCCCCTGCCTCTATGAGGGTGCTCCTCCA
CCCACCTACCCACATACCGACTCCAACCTTCCCATCCTGGTATTCCCTTACACTGGGGCATGGAAGACTCTCAGGCCCAAGAAGGGCCTCTGCC
ACATATGCGACTGGAGCAATGGGTCACTCCATGTGTACTCTTTGGTTGGTGGTCCAGTCCCAGGGAGCTCCAGGGGTTCTGGCCAGTTGACCCT
GCTGCCCCCCCCACTCCACGGGGTTGCAAACCCCCCCCCCCCCTCAGCTCCTTCAGTCCCTTCTCCAACTCCTCCATCTGGGACACTGCACTCA
GTCCAATTATTGACTGTGAGCATCCGCCTCTATATTTGTCAGGGTCTGGCAGAGCCTCTCAGGAGACATATCAGGCTCCTATCAGCAAGCACTT
CTTGGCTTCGGCAATAGTGACTGGGTTTGGTGGCTGCATATGGGATGGAGCCCCAGGTGGGGCAGTCTCTGGATGGCCTTTCCTTCAATCTCTG
CTCCACCCTTTGTCTCCAAATTTCCTCCGGTAATTTGTTCTCCCTTCTAAGAAGCACTGAAGCATCCACACTTTGATTTTCCTTCTTGGGCTTC
ATATGGTCTGTTCATTTTACCTTGGGTATTCCAAACTTTTGGGCTAATATCCACTTATCAATGAGTACACACTATGTGTGTTCTTTTGTGGCTG
AGTTACCTTACTCAGGATGATATTTTCAAATTCCATCAATTTGCCTGGGAATTTCATGAAGTCATTGTTTTTAATAGCTAGATAATATTCTATT
GTGTAAATGTACCATATTTTCTGTATCCATTACTTTTATGTATCACAACTTGGATTTTTTTACAGGTTTAATTTAAGGATCTTTAAAAATAAGA
TGGGGTTAAGATTGCAAGCCTATAATCACATGTATCTGGGAAGCTGAAGCTAGACAGTTGGAAGTTCAAGAATTCCTGGGATACATAATTAGCT
CAAGGTCAGCCAGAGCAACTGACTGATACCTTATCTCATATAAAAAGTTAAAAAGAGGTGTGGACATGTATATCTGTGATTGAATGTTTCATAT
CCTAGGTGCACTTTCCAGTACTAAGAAAGAGAAAGAAAGAGAGAAAGAAAGAAAGAGAGAGAGAGAGGGAGGGAAGGATGGAGGGAGGAAGGAA
GGAAGGAAGGAAGGAAGGAAGGAAGGAAGGGAGAGAGAAGAAGAAGAAGAAGAAGAAGAAGAAGAAGAAGGAGGAGGAGGAGGAGGAGGAGGAG
GAGGAGGAGGAGGATGATGAGGATGATGAGGATAACAACAACAACAACAAGAACAAGAAGGAGGAGGAGAAGGAGGGGGGAAGAGGAGGNNNNN
NNNNNNNNNNNNNNNCTTATGTCTGTACATACGAAATACTAAAGCTTTATCTAATATTTTTCTTCAAGATTACAACAAGAACATTGAACACTAA
TACAGAGAACATCTGCAGTGATGTGATTTGAGGAAAATGAACCCAAAGGCTCACTTATCCAGGCACTTTGAATCACTTTCTTAACTCTGGCCAC
TTAATGAATGTGGGGAAAAGGAAAGAAGTTGAGAAAATTTGAGATAAGACTGTTGGAAAGCAATATGTTCTATAGTTATTGTCATGAATTATTT
TCCCCAAGAGTTTCTTTTATGGATATTGTCCTCATATAACCCAGGCTCATCACACAGGGCAGTTTATCCAAAGCAGAGCTAACTGTGGTTGGAA
ATTTTGCAAATATTGTAAAGTCTTGAGAAGCCTAAAACACATACAGAACAATGTGGAATTAACTTCTGTGAGGCAGGACCATAAAGCCTTGAAC
AACAATTCATATTTATTTAGAGAAACTAGGCTAAAGCAAAGCAAACAAACAAAAGAATTGACTGAGGGAAGTCCAAAGCGTATTTTGTTCCACC
TCCTACCTCAGCCTTGGTAAACAGAGCTCTAAATTTCTATTTACAATGATTACATAAATATTATATGGTGACATTTCTGACTGTGAACCACCAT
ACACTCAAGTATGTAAAGAAAGTGTACACACAGGAATCAATGTGTTCCAAGAAGATGCTTGAATAATTGTCTAACAAAATTTATCCCAGGAGAC
AGACTATGCCAAGTTATAGTACGAAAGTTATAGTATCTGCATTGTACAATAGCACAGGAGAGAGCAATGTTACTACACCCCAAGTTTTTCTTTT
ATGGTAATAATAATTTGTTCTTTAACATTTAAATGATTTATGAGTTGTAGATTCAAAGCATACATTGTGATTTCTCTTCTGTCTTCATTACAGG
ACAACACTGTCCTATTTTTGTCTCCTCTGTTGTTATTATGTTTACATAAAAATCTTTCAAGACAAAATAGTTATGTTCTACATACTCTCTTGCC
CTTAGCTTCCGTAACTTTTGCCATAAGAATTAGTCTTTTCTGCCCACACTGTAGTTATACCAACATGTGACTTTGAGTACCATAACTGAACTTG
AGGCATACTATATTTTCAAAGGGCATTATACATTTTTCAGCACAGGGTATTAGCCCATTGGGATACAGTGAAAAAAAAGTCTTTAAAAACTTGC
TACAGGTTTGAGTTGCAATGTATGCTTTGAATCTACAACTCATAAAGAATCTAAATGCTAAAGAACAAATTATTATTACCATAAAAGAAAAACT
TTTGGGGTGAAGTAATGTTGCTCTCTCCTGTGCTATTGTATAATGCAGAGAACTTGTAAGAAAGTGAGCCACTTGGTAGTTCAAGAAGGATGTT
AGAGAGAATCTTAATTATATAGCTATGTGAGTCCAGAAGTGTGTAATTAGTATATTATTCCATATGTGCTCTTGAGCTTTTAATTTCAAGAACT
GTATGTTGGAATGATTTTGCCAGAGGCAAGTTAATGACTGAAGGAAGGAAGGAAGGAAGGAAGGAGGGAAGGAAGGAAGGAAGGAAGGAAGGAA
GGAAGGAAGGGGAAGAAAGGAAGATGTGACTTACTAGAAGTTGATAGTAGAATTCTCACTGGACTCTAAGAGTTTAAATTTATTACAGGTTCTT
CCTCCTAGCCTCATGCTCTTAGAAATAAGACTCAGATTCAAAATATATTTTCATATACTTTGGCCATGTGGCTAGGCTCTTCTCTGACTAAAAT
CATAACTTAAAACCAACCCATTGATTTTCATCTACATACTGCCACATACCACATGTTACTTGTGCTATGTGCCTGTCTCCTCACATCTTCCTGG
GTGGAACTCTTGTCTGGCTCTATCCCAGAATTCTTTCTCCTCCCAGATGTCCCACCTCCCATTTCCTGCCTAAGCCATAGGCCATAAGCCTTTT
AATTGACAGGTGATGCACCCATACAGTACACAAGATAGTCTCTCTACAGCTAAGTTTTGACATTGTTTCCTAATGTAGAAATTATTAGGAGCTA
ATTTCTGAAAAGTTTTAAAAAGTTATGTAGACTGTCCATTAACTAACGTTTCAGAAAATTGTACACAGAGAACTCTTATTTCCTTTTGCCTAGT
AAATTACCTCTCTACATAAGAGCGTTAAACCAGATATGTATGTCATTGATAATCACTATATTTTTTCCGCTCTTTACATCCATTCCATTAGAAA
ACTTTATGGCTTTTTGGCTCCATTTTCACCAGTCTTACTGCCTCTAATCCTAATACCTTTTTGAATGGAGAACTGTTAGATCTTACAGGCAGTT
CAAATATCCAGAAATGAGACCAAAGTGGACTATAGGATGATTTCCAGAAGTTAGATAGAAACCAGGATTCCTTAGAAATTTGTAAAGCCAGTTC
CTATTTACCAAAAGGATTCACTTCTCTTGTCCCTGGCACAACGGACATAGAGCTCCACCGACATGTGCCTGTGGTTGCATTTCAAAGCCCATGC
CATCCTCCAGGCTCCTTCAGCCCCTACTGAAATATACTTGTTACACATTTCAAAGGCTATGCCAACCTCCCCAGACATCCATCCTCCTTATACC
TATTTGTTCTCTTTTCAACATGAATGATTTTCCCCTTACCCTGGCTATTTTCCCTATACTTTCCCTCTATCTCCTGCTCTCCTCTTTACTCTCT
CACTCTATGGGCTATTCTACATTCTCTCATAGATGCACTGGCCATGTCCAGTCTGCTTTGATTCTCTCTACTCTCTCTGATTCACAATAAAAAC
CTTCTCCTCACCTGTACCATGGAGAGGACATTTCAATTGTTTCCTTACTGTTTCCCTTGCAAAAGGAAGATCACCCCAAAATTTGGTGCCAATT
CTTCTACTAGTAAGCAATCCATAAATATTCAATTAGTGCGTATATAGACAAATGGACTTCTCCTCCAAATTAATAAAACTTAAAATATAACAAG
TTAAGAAGGCATGTAACATGAGGAGCTGTAAAGAATTCAGATAGCCTTCTTTGGTATTTTAATGGAAATCTGTGACTCAATTTACATGTGGAAG
TTCTAGTACACACATGCTTCATTTAGTTAAATACGTATACAGGTGGAAGTTACTATATCTCTTGCCCTTTGCCTTGCAAATTCCACTCAATCAA
TGCCAAGAACTGAACTAAGAAGACATGTTCACCTCTCTTTCCCTTCACGTTCAATGTGACTCAGTTGTGACCCTGAAAGGACAGTACATTCCAT
CTGTCTCACACCTGCCTGGTGTCTGGGATCCATCTCTGGGATTGACAGCATCTGTGTCCTCTGATTTCTACTCTTTAGAAAAAAAAAATCAATG
CCCACATCATATTGGTAACCTTTCAAAAAGCTCAAGAAGAGTGACAAGAAAGCAAAATTGCTCCCAGGGAGCTTCAATTGCTGGTGAAAGGCTG
ATCCAATAAGACCTCCTGGCAGAAAGCACAGACATTCCAGGTCATTCCAAATGCCTGTGACAATATGTCAGTACACTGTCTGACTGCGTGTGTA
GTAGTGACTCTTAACATGGTTATTTACTAACCACGTGTTGCCATGGAAACAAGGTAAAGCAACATGCTCTTGAGACACAGAGACTTAGCATGTT
AGAGAGGTGGTGCCTAAATCCCATTCTACAGCCAGTAGGCTGTTCACACAGGCAGACCACACCCCTCTAGGCCTCCTATTCTTCCTTGGGACAT
GAGAACCCTGTAGCCACTCATCCTCAAGACATGGTATCCGCTCTCCTCTTGTATTTGAGAAGCATAACATTCTGAGGCTAAAGGGCGGAGCCAG
CCATCTTCTTGACCGGTTGGGCTTACAATTATGTACTTGACAATCTCCAACATGTTTGAATCAGAGAGGAATACAGTAAAATTCATTCATATGA
TGCTCTGGTACTCCTGCTTATTGAAATAGAAAAAACAGACATACATACTTAGACTCTCAACGTTTGCAGGAAAAGAAGTTGAAAATATGTTCAA
GAGTTCAGATGGCTTCTTTGATTCGTGGTAAGTTTGAGTTCTATGGATTTGGGTTACTCGAACTCTGATTGGTCTCTGGAGTACATGTAGACGT
TAAGATGCTATATGCTCTGAGAGAACAGTGAGCTGAGAGGGAAGCCTATAAAGTAGTGACACTCAGATATAGAAACATTTTCATATGATAGTAG
TAATGGTGCCAGCACTTCTTCCACCAATGTCACTGACAAGCTTCTTGTTACTGGAAGGAGAAAAAGTCATCAGACCTTATGAAAAAAAGGTGCT
AGGAATACATGCGCAGGGATTTTCTTATGTGCAAGATTCACTGAGCTCTTTCTTATGACCATATAACCCATCTACATAAAAATATTATTAGGTG
CTAAATCCACAGCAACTCAAGCTTTGGAGCCTTGGAAAATTGGCTTTAGGAAAAGAGATCACCTGGCAAGCTCCATGTCTCCCTTTCTCACAGG
ATTTTTGGAATAGGATTAGGGTCAACAGTGATTTTTAAGTCTCACAAAGAAAACAAAAATCGATTTCTTGCAAAACTATCGCTGTGCAAGTTTG
GTTTTCCAGCATAGATCCGGGCTGCCAAGAATGCTGGCTCCAGCTAGGTCTTCATTAGCCAAGTCAGGGGATAGTCAGGGCATTCATCCAGGTG
ACCTGAGCCAACCCCACCCACTCAGAAAACCAAGACAAGTTTGCCTGGTAGAGCAAGGGGCCTCACTGGCTGCAGGCTGTGACTCCATGTGTCG
TGACTGACACCACAACCAGGTATGTCATGAGAAAAACTTTGACAAGCTGGATGGCAGCTCGTCGTTCCTTGGATTTCTTCCTTAACTTTGCTTT
CTGTGGCTAACAACTGCCATTGAATATAAGGTTATGTAGGAAGAATTTTAAGTATGCCTTAAGAAGTTAGGATTGTGTCCTTTTGTAAAAATGA
TAACCCTACAGAGGTCCCTTTCATAGCATTAACCTGTGGTCGTGTAAATGGGTACTCAGCCTTAGAGCCCATCTCTTTAGAAAGCAAATCATTA
TGCAATCAGACAACTTTAAAGTTGGAATGAAGTTACCACTATCTCAATTAGGAGTAAGGGGAGAGTATAGTCTGTAGGAGTTATGCGACTTTTG
AGGGTAACACAGGTAGCTGGGCATGCAACCAAGCATACAATACTGGCTGCGCTCTCATTCAACGTATCAAGAGTCTTCTCTGATATGTGACACA
AGCAAATAGAAAACTCATAGCCTCCAAAGAAAGATCTTAGATTCATCAGGTATGTTTGTATGCATATGTGTGCATGTGTGTGTGTGTGTGGAGA
GAGAGAGAGAGAGAGAGAGAGAGACAGAGAGACAGAGAGACAGAGAGACAGAGAGACAAAGACATTGAGGCAGAAAGAGAGAGATAGATGAAGC
AGAAAGAGAGATAGAGATATAGAGACACAGAAACAGAGAGAGACAGAGAGACAAAGACAAAGGAAATAGTAATACCTTGGGTTTCTGGCTATAT
AGTCTCTCTCTCTCAATTTCTTGTCATTGTAGCCTGAAAACATCTAAAAATCACAGGAACAGGGATGACTGTGCCTCAGCAAAACTGTTAATAA
ATATAAAAATGTGAATTTACAGCAGTTTCATGTGCTATGAAAATTTATTATGTTTTGAGGGGTTCTTTTCTACAACATAAAACCCATTCTTGGC
TCTTAGTGGACCGGATTTTGCCTAGGACAATAGTTTATCTGGCATAGGGTAATAGAACCAGCCCAGGAGAGAATGAGCTGTGGACTCTGTCCTC
CACTCGGGCTCTCTGACCTTGTAGCTGACTCCACAAGCCCCCTTTTCCTTTTCAAGGGTTCTGTCACCCTACACTGTGAAATTAGTCACTGTGT
GTCTCTTCCCAAAACATTCCTTATTTCTTAAAGTCAGTATAATTACAAGGCTTGTTTAGAGCACGGTCATAAACGTCATAGTGAGGACTGGAGC
AAATGTTGCTTTGAACATAACTCTGTGACTTCAACTAGGTGGCTATGGTAAAAAAGGAAGTACTTCCGTTTGCCCATAAACAACTGTCTTAAGG
AATTTGCTACATTCCTTGAGTCAGCTGGATCTCTGGACAGGCCTGGTTGTTTAGCTTACTAACAGCCAATCTTTCTTTCCGACTGGCCCTGAGT
ATGTGTGTCAGTAGGACAGAAGGCCCTCTGGGTGAGAGCAAGCAAATGAAGACCACTGTTTTAACTCAGATACAGGCGGTGGGGGTAAGTCTCA
GCCAAGACAGAAACGCTTTTCCCTCAGCTGTGGTGTGAGTGTTTCCTTTGCAAACTTTGTTGAAATGTTATTGATGTTGTGATAGTGTTGGTAG
ATTTAGTGTCTAACTGGTGGTGAACTGGGTCATGAGGTCTCTTGCCTTATGATTGTCTGCAGTGTGGAGAATGGGTAAGTTAGCATGGGAGGAG
TTCGCCAAGAAAGGGAAGGCTCTCTCTACATCCTTTCTCTGGCCCCTACCCTCTCTGTGTCACATAGGTTTACCTTCCTTTCTACACCAGATGA
CACAAAGTTTGAAAAACTTCAGAACCATTAGACAAGTAAAAACTTTCCCAGTCTCCCCCAGGTTTCTATAATAGTTGATAAAATTGGCTATGAC
TTGTTTTGTTTATGTTGGTGTTGTGTTTGGCATATGTTTTGGTTCCTTGACCCTCAGACCCAAAGAGAGAGGTGGTTTTCTCAAGGCCAGGATA
AAGGCACAAAGACTTTGGCTCAGAGCCACCACAGAAGAACTGTGAGAGAGTCCCAGGAGCCATAATCCAGGCGTCTTCCATATATGAAGTCTCC
AAAATCAACTAATATTGCTTACACTAATATTGATACCATTCAACATGACAACTTCTTAAAATCACTGACAGTGATGCAGTGTGGGGCCTATTAA
ATATAGTTATCAAAACTTTGAGTTGCAAGTGGCAGAAACCAAATGCAAGATGAGACATATAAGGAAACGGCCACATCAGCTTTAACTTTGGAAC
CAGCCATAAGCATGCCACTGCTTACGCATCTCTTCCCTCTCTGCTTTTCTTTGCGTTGACATCATGCATCATGTGGAGAGACACACAAGCTCTA
GCCTTCTGAGGCGCTTAGAATCCTGATAGCTCACAGTCTCAAAAAGGAAGAGATTTTATCTCCACATCTATCTCAGTAGACAAGATTTTCTGTC
CTTGTTTCAGACATTGCTCACTGTCAGACCCACCAATGGAATGGAAAAGACCCTTTTTGGGGTGGATTTTTAGATTCCCAGTGATCAGCATGGA
GCAAACAATGGGAAGTTGTGTGTGAAGGAAGGGGGGGGGTAGATGCAAGGCTAAAAGCAGTGCCTGCACCAACTGATTCCTGTATCTAATAAAA
ATGTGGCTTTTAAAAACAGTAAGCATCGTAGAACCCACCCCATGAGTGTTGAGGTGATTCTGGATAATTCTTTCCTCTCTCATTCCTGAGCCGT
CAGTCTTCCCTGGTGAAGACCTGAAATAGTACAAAAGACGATGAATGGGTTGATACTATTGTGAGAGTTTGGTATAAAACTCAGCTCTCTTTTT
GCTCGCTACACTCTTTGATTTCTACCTTTTGTTTCTACCAAGAATGATATACCAAGGAGACATTTACTAGCTGTGAATTCCTGGACTGTGGAGT
TCACAGCACCCAAAGCTGTAGGAAAAAAAAATGTATTCTTTAGAAACTACCTAGTCCTAAGTATTCTGTAAAGAGAGCACTAAGCAGGCTGCAG
TGGCTGTGGCTCCTGCAGAGTCTTACTTCCACACAGTGACAGGGGTATGTGCATTCATCGTCCCCTGCTCTTAGAAGTTGGAATGCAAATCCCT
TGAAGCCAGGGCCAGAGTCACTGTTCCACAGGGGACTGCCTTCCCTCACAATACAGGACCATCACTTTTAGCACAACCCACAGGTCTTCTGAAT
ACTCAAAAGCATGAAGCTAACAGACAGAGCCTTATATGGTGCTAAGGACAGAGTCAAGTACAGTGTTAGTTAGACACCTATAATCCCAACACAG
ACACCTGAGGCTTTTCCTTTAGAAGAGATGGGAAGCCAGGGGTGTGGAAAATTAAGGTTTTGTTCCTGCTCCCTTCTCCACTCCTCCTCCTCCC
TCTTCTTTTGGTGTTTTTGCTGGATACTGTGTTCCATGATGCAGTTTAAACTACCCCTAAATTCAAAATCCTCTTGCTTCAGCCTTCAAAGTAC
TAGGGCTATTGTCATATACTGCCATTCGCCAGCCTGTCAGTTCAGCTGTTTGACTTTTAACTGGCACATACCATTATATGTATTCATTGAGTAC
ACATGTTATTTGGAAAGGTATAGACTATAGAATAACTGGCTCTACAGTGTATTGCCTCAGTTCTGTGGTAAGAATGTCTTATATTAATTGCCAC
TGTGGTTATACCATGAACATATTCCCCTCTTACAACTGAAATCTTGTTCCCTGTAATCAACATTTTCCTACCAACCCCATAGCCTGGCCTGCAC
ACTCTTAGTCCTCTTGCTCTATACGAGTAGTAGTTTTAGGTTCTACATAGAAATGAGGTCATGTATTTGTCTTCCAGTGTCTGGCTTATTTCAC
ATAACACTCTGGAGTCATTCTTGTTATCCAAGTCTTTCTGTAACTGAAAAGTATCTCACTGGGCCTGTGTACCACATTTTTATTAGCCATGCAG
CCATGATGAATAATCAAGCTACATTTTTGATACAGCAAATATTATTGCAACCAACATGGGATATCTCTTTGCCATATTGACTTTATTTATTTTT
TTATACACAACTAGTAGTGAGATAGCTGGGTGATGTGATGTGTCTACTTTTAGGTGTCTGAGGAAACTCCATAGTGTTTTTCCTAGTGGTTGTA
CTAATTTACATTTCTACCAGCAATGTGCAAGAGTCCCTTTTCTCTTGTATCCTTGGCAACACTTATAAATTTTATCTTTTCAGAGTAGTGGTTC
TAGCAATGAGGTTGTATCTCATTGGCATTTTGACTTCCAGTTTCTCAATAAACATGATGTTAACATTTTTAAATGCACATAATAGGCACGATTG
TTTTTTTCTTTTGAGAAATGGGCATTCTATTATTTATCCATTTTGAGCCATACCCTTGTTATTAGATTGCTTTGTTCTGTATGTATTTTGAGCA
TTAAGCCCTCCTCTGATGTATAATTAGTAATTAGAATTCCTTCTATCCTTTCATTGTTCACTGGCACATTTTGAAGAGATGTGCTGTGACCTAG
TGCTGGCTTTGAAAGCATCACCCTGCCTGTTCTATTGGAGACATACAGTCTTCAGAGGCACACAGAGAGACAGGAGAGCAATGAGGAGCTTGGT
GTGATCTCCAGGGTAAAGACCTCCATTTAGGCCAGGTTGGTGCTCTAGATAAAGGTAAGAGGAAGCTGCATTCGCTCATCTCATTTAAAGACAG
AGGAAGCAGGTTTCGCCAGTTTGAACTGCCAAGTTGTATGGAAAGAGATGAGAAAAGTCAAGGATACCATCAAGGCTTTTTGTTCTGAGAAACT
GGAGTAAAAATAAACATTTGTAGTTTACTTTGTTCTTGACTCACATGAAAGGGAAGTATATTTCTGTGACAGTTAAAATAAGTACATGGGTACT
TTGAAGTATGGTTTTAAAACTGTCTTAAACTAAAGCCATGAACTTTTATTGTTCTGCCTATGCAAGCAATGTAAAAAAAATGCATGCATATGTA
TTTTTCAGAATACTAAGATATATAATAATATAATACACACACACACACACACACATATATATATAGACTGTTTGCAAAACAATGAATTCTTTTC
TGTATCATTTTATAAATGATTATGTGTCCAGGAATCAGAGTAAAGCTTTTTGGAGATGCATGTAGCAAGGGTTCCTTATTAATTTCATAATTTA
ACACATTGAGCTAATTGGTTTGCTCACAAGGGCCATAGCTGTTAGTCACTGTACCTTTATTCAAGAGGAGAGCAGCTAAAGCTGGATGGTGCAG
TAAGATGAACCTTGGTCCTGCAGTCTGTGCATGGAACTGACCCCTCTCAGCATCAGACTCTCCAGCCATTCCTTTCATTACTTTGGACCTCGGT
CCCACATCTTATAAGTGAAGGTAACAGAAGCCAGTGTCACAGATGCAAAAACAGCGTCTGCACTCCAGGTGTCCCCGATTACTAATGACCTCCA
CTGGGAGCAGTTTAGTTAAACAAATATTTGGTTCCAGCTGTATTCTAATTTATGTACTTGTTCATGTTCTTCTTTGCTATAAATGATTGCTGAC
CTTATTAACTATGGCCCTTGTGAGTGAAGATTGAAACCAATGAGCTTAATGTGTTAAAACTGTGAAATTAATAAGGAACTTTTGTTACATGTGT
CTCCAAAAAGCTTTATTCTGTTTCCTGGGCACACAACGATCTGAGCATCAGAAATGAGCAGAATAAAGCTAGACACTGTAAATCTATCATTGCT
CCTGAGAAAACTTAATTAAAAATATACTCCAATGTCTTTTTCATATTTGAAAGGTACAACCTCAGATTATTAATTTATTATGGTATTTTACTTT
GAGACAGGATCTTATGTAGCCCAGGTTGGCCTGGAACTCATTATGTAGCCAAGAGTGATCCTGATTCACCTACCACCAACTCACACTTGCTAGG
ATTATAGGCATATGTCTCCATGATCCATAGCAGATCCTTTAAAATAATGGCAATGAATAAATAAGTATATAAGCAGTGAAGAAACAGGATGACA
GTGTGAGACTAGATAACCAGATAACCCTAAACTCTAGACACTGGTATGTCTGTGTACCCTAAATTATTTAGGCTTGTGGTGCAAGACGTCAGAA
TCCTTCTTTCCTCAGATGAGAACATTGATGTCTTCATGTCTAACGTGTCTGTTTGCATTCAATGAGATTATGAATATCTCCCTGGGAACTGTTG
GCTCTATTAGTAGGGAATATATATTCACATGTTTTATTTTAAACATTGCTGTTCAGTTTTGGAAGACTGTAGCTATTTGAAGCTATCCATGCAC
TCTATTAGAAGTGGGAATTTATTTTTAATTATCAATTAAGAAGTCATCCATTGTATGTGACTGGCTTTGAACAAAAAATAATAATAATTAAAGC
ACACACACACTGTGTAGACAGACATTGTTACAATGCCAGTAAAGGGAACAACCACAGCACATATGGCATGAATGTTGCTTGAGACCTGAGTATA
ATCCTTAGGTCTTGAGCAGAATGAGACAGCTGACTTCCTGTAGCCGGAAGAAGAATGAGTGCTTGCTGTGTACAATGCTCAGGAAGGGCTGACA
GAGACACAGGCGAGCAGCGGAGGTGGGGAAGACAATTACAAAGGAAGCAACGAAATCTGAACAAATCAGAGACTCTCTAGAAGATAACCCAGGT
CTCTGTACGAATAATCAGTCTGTCAACCACTTCCTCTACCTGCTCTCTCTATATATTTTTATTAGATATTTTCTTTATTTATATTTCAAATGCT
ACCCCAAAAAATTCCCTATACCCTCCCCCCCCCCTGCCCTGCTCCCCTACCCACCCACTCCCACTTCTTGGCCCTGGCATTCCCCTGTACTGAG
GCATATAAAGTTTGCAAGACCAAGGGGCCTCTCTTCCGAGTGATGGCCGACTAGGCCATCTTCTGCTACATATGCAGCTAGAGACAGGAGCTCT
TGAATTGGAGGACTCTCTGCTAATTGTCTTGTCTATACCTGATAGCCACCCTTCACTAAGCACTCTCTGAAAGTCAGACTTTATATACACAATC
TTCTCCTCTCACCTTTAGTACTATGTCGTTATCTCCATATTCAAATGAAGGAAACTAAAGCCAAACATTTTTCCTAAAAGACAAACAGCAAGTT
TATGGAAGACGTGTAACCTAAGGCTGGGATTTTCCAGGAGCAGCTCCCATGCCTTCTTCAAGACCCTGGCTTGCCTCCATTAGCAAATGTACAC
ATCACGTGAGCATGTAGACCCCAACTGGCTTCTCCACTGTGTGCCCCGTCACCTTAGGAGTGAGAAAGTAATGATACTTGCAAACAAAGCATCC
ACGTGTTACATTATCTCCCACACACACTACAAACCACACACACTACAAATACTTAAAACCAGCCCTCAGGCTTAATTAACAAGAAGTAAACTAT
ACAATTTTCCAAATGATATCTGGACTCATGTACAGTGAGCTCATATATTCATTTATTCCCAGAGCTGTATAACTTCAAAATTAAACTAATTAAT
TCCTTGTATTGTCAAGCAAAACATTGACAATTTTTTCCTGTGACCGCCTGTTCCTAGCATGCTTGTTCTTAAATGTGTAATGATATAAAGTCTT
AGGTGATTGTTTTTGAGTTGCGTTGACTTTCATGCTAATTATCTGAGTGATGGACAATGTGTTCCATCTCCTAACTCACATGGCCTAATAGACG
TGTCAATCTAACACAGTCATGGCATGAGTGCCAGCTGTTTTTGAAACATCGATCTGTCATCTCACAAGATGAGCCTCCCAATTTTCCTTAGTGT
ACTTAGTCATGTGTAACTAGGTCTTAAAGTACCCTAAGTTTCCTTGAGGCACAGACACATGCAATTATTTTCATCGTAAGTCTTGCCTGGATGA
AATAATTGAACATGAAAAGTTGCTGCACCCCAGTGAATCCAGTTTATAAAAGCGTGTGTGATGAATGAGATAGGGCAGATTCGAGTGATCGTTA
TGGAGAAAAGGCCATTCAGATTAAGCAAACTGACTCCTTCAGTGTGTGGGTGCCACAGAAGGCATTCATACCAGAAAGCTGGAAATAGTAATTT
TTCCACCTTCCCTTGATGTTTATGATTTGTGCATTTCTCCAAAACATCTTCCTATTAGGATGAACTCAGTCACAGGATGATAGCAAACTGCGTG
CACCAATGTGTCCATGTCTCTGGCTACTTTATAACAAACCACCCTGGACTTCATGGTGCAAAGCAATGATCATTTTATTATACTCATGAATTAT
GTAGGTCAGGAAATCAGACACAGCCCAGCAGTGACTCCTCTGTCTGTTGTGTAATGTCTAGGGTCTCAGCTGTGAACAGTCAAAGACTTGGGAA
CTCGAATCACTCTTAGTGTCTGTCGAGGATGATGTCAAGAAGCTGGGCTTCTTGGCAAAGGCTTTTTTGAACGTATGCTTTCTGCAGGGCTCGC
CTCTGAGGCCTCTGAACTCTGAAGAGGACCATGCAAGATCACGTGAGATCTTAGAGCAGATGCACCTGAAGCATCGTGCACGTGCACATAAGCG
GGCCTTCTCTAGTCTAATTTTGGCATCCTTGCAGCAATGGTTCACCTGGATCAGGGAAGCACTTGACTTACTTCTTAATGGGTGAAGAACAAGA
ACATGTTACTAGAAGTTTATTCAAATTGACTGGCCGTGATTACTGGTCAGCAACAGCTGCTGTGGAACTGTTGCAGGGGCAAAGGCAGTGTTTA
GGGTCACATAGTAATTTAAATATCACCTCTCAGGTAGGACCTACATCACTTCTGCCTGCATTTTAAGGCTTGTGGAGCCACGCCTAGATTCATC
ACGCAAGGGTGGTGTCAACTGTGTACTCAGAAGGAAAAGAGAACTACCAATATTGATGAATGGTGATGGTTATGACCACTTAAAAATACTGCCT
CCATGATGAAATGTGGATGTTTCTTTCAAAGCTAGTTTTTGTAATTATTTGAATCAGTTCATACGTGTCTTCTGCCATCCACCAAATATGCTTG
ATGGGCAATGCACGTAACATAAGATGCTGGATCCGGGTGTGTATACAGCCCCCATGAAAGGCAGGAAATCACTTGAAATTTCGGTGTATGGCCT
TTTCAATTATTTCTGTCTTTATCTCCCGCAGTCATTTACTATAGTTTGCACACCCTACTGTTAGTCAAAACCCTGATGTCATAGTCATCACATC
GGTACCCTTTTGTAATCTGTGATATTGTATAGAGTGTGTAGCTATAATTAATATGGAATTGCAGGTAGATACTATTAATTGAAGTGTCCCGATT
AATTTAATTATTTCTTAAATCATTTCAAACAAGAGTTATCTTACATCAAACTCTGATAGATAGAATGGTTCGTTCTTACTTTCATTTAAAATGT
ATATAGATATTAAAAGTACAAGTAGTGGATAAATCCAGGTATAAAGCAGCTTATTTTTCGAAAGCAGAGCCTGTGGCTATACTTCAACAATAAA
AAGAGCTAAAACCAGTGTGAGACCTGCTGGGGACCAGTTTATATTTTTGTCTATAATGAGAAAATCATAGTAACAAATTGTCAAACAATCCTGA
TGCCCTATGAGAAACAATGGAGAAAAGGCAAGCTCTGGGACAGGCTCAGAGGTGACAGGGTGGATTTAATTTACTTGTAGTTTGACATCAGGGA
TGCATCTGTGCATCTGTACATGTAACAACTTTAAATGCCCACCCTTCCTTTCCACCTTGCTACATCTGGTACTGTAGAACACTGCATACTAGCT
GGCCTGGGGACTCTGAGGATTCCCCTGTCTCTGTATCCCATCTTGCTGTAGTAATGCTAGGATTACAGACTAGGAATGTAATCGTGTGTGTTTT
TATGTTTGGCTTCTCATGGGTCCTAGGATCCATTCTTCAGAGTTGTAAAGCAAGGGCTTTGAACATTGAGCCATCTCCCAGCCTTGGGGAAATG
TCTTCACAGGTAGCTCTGGCTATGTAGAAAATTGCTGGCAAGCTTAGTTAATACAGGCACTAAATTGTGCTGCAGAATGCTCCAGTGAAACTCT
CTATGGATAACTTGGTAAAAGATCAAGGCACTTCAGTTAAACAATTTACCTAATTACCTATTTAAAGGCATCTAAAAAGAGATAGAACCAAATG
AACCCATTGGAAGCATAGACTAGAAATGCAGTAATATCATGAACATGTGTTTAAATAAATGTGTACATGGCAAAAGCCATACAGCAGCATGGCT
AAGCTTCTTCATCAACATGTATTCAAAATTATGAGAAAACCTATCTCAAAAGTTACATGAGACAACTACTTGTCCAGTTTTCTGTCCTGGCACA
TCTATGTTGAATAAAACCTGTCAGTATAGGCTTATGATCAGAGTCGTAAATTTGCTAAAACAGGTACCTCTTCCCTAGGTCTGTCAAACATGGT
CAATCAGTTACAGCTTAGGGCTTAAATCTCATCTAAGGCTGAGTCAGAAGAAGTTAACATAGGTTCTATCAAAACCACACACAATGCTGGAGAT
AAACATTGTAACAATGACTTTGCCTACTATGATAATGAAATGTCAGGTCTTTTGAGGTCTCAGTCAGATGTAAAACAGACTAGATACTGATTGT
GCTAACAGAAATTTACTGAGGAACTCAGCATTTGTCTCTCAGGAATCTTAGCTACATTGTGGCAGACAATGATTATCAGGTTCATTTACTTAGT
GCTTTGCGGTCCAAAGCACACACCTCTCATAGCCCAATGCTATATTTCTTGTACCCTCCCAGAACTTTGAAGGCTGTCATGGTCACTGTTCTAT
AGTAGTGAAGAGACAATGTGACCAAGGCAACTATTATATGAAAGAAATCATTTAATTGGGAGCTTGCATACAGCTTTATAAGTTTAATCCATGA
TTATCATGGTGGGAAGCATGGCAGCAGGCATAGCAGGCATGGTACTGAAGAAGAAGTTAAGAATAAGTGGGCCTAGCATGGACTTTTGAAACCT
CAAAGCCTCCCTCAAGTGACATACTTCCTTCAACAAGGCCATATCTTCTAATCCTTGAAGTCTTTTCAAAGAGTTCCACTCTGTGTTGACTAAG
CATTCAAGTATACGAGCCAATGGAAGCCATTCCTATTCAAACCACCACAAAGGCTAATCCTCTCTCAAAAACAAAAAAGATTATGTATATGTGT
ACATTTTCATAAATATGTCTTACATATTTAAAACTCTGCATGTAGATATAATTCTAAGACTTCCTAAACATGCAGAGGGTCTATAATACACTTT
GGCTAGCTTGTGTTCAAATGACATTTTGTTGTTGTTGTTTAAAGCTAGGAATTGCCTTCTTGGAAATATCAAATCCCTACAGAACTATTAATGA
AAAATAACTATCTCTTTTAAAGTAATTTTTTTAAAAAAAAAAGCTTCTCTTAGAAAAGAAAGGGAACTTTTTCTGGGGTGCATCTGTAAAATTT
TCTTCAGTCCTCAAAACAGTCCTTTACCAATGAAAACTCCAGACAGCAAAAGATAAGGTTGAGCTCACAGGACTGGTCAACCATAGGGATTCTA
TCACAACTCTGTGCTGCCTGCATCAAAGCCCCAAGACCCTCCAGCCTCTCCCCTCAGCCTCAGTTCTTGCGCGCGCCCGACTGGCCAGCAGTAA
CGACGCTGTAACAGGATCCTTCTGCACACGTTTATTGGGAGAGCTTGATTGCACAGGCGAAGAGACCCTGAGCCCAGAACTGGTGCTGCTTTTA
TAGGCCTAGGAGAGGCGTGTCTCATACCCGGATTGGTTATGCACTACGCCTCATTTGCATGTTCCTCATCTGATTGGCTACTCTCTCTCTCTCT
CTCTCTCTCTCTCTCTCTCTCTGTACCTCACAGAGCCTCATTATCATACCTCATTTGCATGTCTCACATCTGATTGGTTATACTCTCAAAGCCT
CATTATTATGCCCAGGCCAGGCAGTGTCTTTGCAAAAAACTTTACTGCATATGTACACATTGGTTGTTTGTCCAAACTTATGCGTGGTGGCCAG
CAGTAGTCAGTGCCACTCTGCAACGGCACATGNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNATATGTTT
GCGTGCAGCTTCTACCTCATTTAAAACTTGCTAAGACCACATGTATCTGATCTACATATATTCCCTGTTATATCACAGATGCTAGGTGTGATAT
AGCACACAGTTGATACCATCCCTTCTCAATAGTTACTGCTAGCCAATTCCCATTGAATGCCTATGGCAATTGACTATTTTGTTAATAGAGTTGG
ACTCTGTCAATCAGTTGAAAAAGTCACTTCGTAACTTTTCCACTATGCTAAAGGTCATGGCAAAGTTGTACCAGGACTTATGAAGGTTCTTTGT
TAGTCTGTTCACAGTAGCAGCACTAGAGAAAATCTTATGCAGTTGTTATGAACAGTTGTACTTAAAAATCAAAGAAAAACTAGATTAATTTGAG
GAAGTCTCTCTTCACCAACAGAGTTTAAATGCCCTCCACATTAAGTGGATGTTTTTAAGTTTTCCATGCAAGAATTCAAGTTTCTAACTCTCAC
TGGACAAGCTGTCTTATTCCCAACTCTTCCTTAGGATCTTCTTTTTGATTATATTACCATAAATGTTTATTCAACATGTGTGATACTCATGACT
ACTTACCATTGAGAGAGTAAGTGGCAGCCTAGCCAGTCCAGGGCAGCCATGTTTCCTTACACTGATGAGCGATGCCCTTAATCACCTAAATTCC
AAAACTATTACACTTCCCATGCAGCATTGAGCATCAAACCATTCCTTATAATAATACCTCAGAGGTCTGGGCCCTTTAGCTCCCTCAGTGACAA
AGCAATGACACTAAGCACTGTACAGGACCGATCTGGCTTTCAAACTAAAAACAACTATAGACAGTAGAAACAATGGCCATCATCACTTTTCTTT
CCTCTTAGGTGAAGGAACCAGAGGCTGATAAAACTTAAACCCCTACAATAGAAATTAAATAAAACAAATTTAGTGAGTCACTATGGCCCAGGAA
AGGCAATAAGTGGTTAAAAGGTAAACTGCTGTCACGATGCTTTCATTACACAGAGATTTTAGGGACATGACATGAATTTTGGCACAGAAAATCG
TGTGTAAATATCACGTGGGCTATGGTGAAAGGCTTAACTATTTATTTTCCTACCTCAGTATTAATTACCTGAGTCACATATATAAGAATGGGTG
AATAAATAGATAGATAGATAGATAGATAGATAGATAGATAGATAGATAGATAGATGAGTTCTTTTTTGTATAGTTTATAAAGAAGTTCCTAACT
AAAAACAAGTTCCAAAGTACTGTGCTCTTTTATGGACTTTCAATCAGTCTGTAAAGCTTTTGTAGGGCCTATCATGAGCCAGGCTCGGGGCAGT
TAGGAAAATGCAAATTTAATAAAGGCTTTGGCCATTAGGAGCGTGTGTGTCTTTCTAAGTTACGTCTATGCAACAACAATGCTGTCTGTACAGT
AGAGTAGAGGGCTCAGAGAAAGCAATGGAAATTCACAGAAAGAGGTGAAAGCCCTTGTAATTCATATACCCCAGAGAGGAACTTAAAGAGTGTG
TTTAGTAGGAATAGGCACCTGCCAAAGAATCTATGAACCCCAAGATATCCAAACTTCTTATAGTCCTGGAAGGAGTTTCTTCTGAGTTGGCTCA
GTTGTGCACTGACCGCTGTTTCCTGCTACATCTTCTCAGAGCAAATAGGAAAGGAAAAATGCTTCTGCTCTGTATTGTATGCTCCTTTCATTAA
ATCACCCTCGGGACAACTGAGTGTTCCAGAAATAGGTGTTGGTTAAAAAAAAAAAAAAAAAAAAAAAAACCAAAAAAACAGGTATTTCTGTGCA
ATCAGAAAGTGCCTCTGCTTTGTTACCAGCATAAAGCATTCATGAAATCAATTTTCAGCACCCACTGGATAGGGCTGATGTGCGAACTAGCTTT
GCGTAGCCCATCGTCTTCTTTCCGAGATGATTTGACAGGACTGGCATTTTATCATGAATACAAAGAGGATGAGCATATTCTCCTAATATATTCA
TTACTTCCCTCTCCGAACTGGTAAATAATATACATATGGTAATTTGAGAGACATAGTGTTCCCCATTGCTCCTTTTTATTCCTGGAACTCTGGC
TGTCTTTGTAAATGCTGCAAAAATCAAAGGCAAAATTACCTCAAAATTTGATTTTGGAGTCCTTCTAGTCTCACTTTCTGGTGCCCACACAAGA
GCTATTGCTTATTTTCTGTTCTGACCTGTATTATTCAATAGCATCACCCCTTCTGCTTGCAATCTTATTGTAGAACCTTCTAGAGTGACTCAAA
GAAGCCATCTATTTATTCATTAACAAGCACCTATTAAGTACCTACTCTGTCCCAGACACTGTGCTAAATGTTCAAAAGAATAAGGACCATGGAA
GCCTATGGCCCAGGCCCATTCTTGTTTTCTCGAGTTTTGTAATCATTTCTCACACAATTATAAAAATCCATGGCATCGTAGAAAGATAGGAAAG
ACTGTGGAGTGGATGCCGTAAACAGAACTAAATGAATAATGAATGACAAGCTGAGAAAATGTGAGTGTTTGCCAGAGCATGTGAGTCTAATTAG
CAGGCATAAACTGATATGCTAAACCAAAACGTGATGAGATTTTAGATCCAAAAGAGCCATTTTTATAGACTGGACCTGAAAGAATTGGATTTTT
AGATGGGAGTATGAAGGCAGATCTGAAGGGCAGTTTTCTTAGAACTAAGTGAAAACGAGATGTTTTACTCTGCACGGACTTGATTGACTTGCCA
TGGCTTGAACGATCAGAATACAAGTCAATTGTGATCAGTAATTTCTTCACTTGTATACTTAAAAGTGTTCATTGACCTACTGCGTTAGATGAAT
TGGTCTTTGCTGAAAGCAAAGGAAATTAAAGGTCTGCTCTTCTTGAATTTGTGTTCAGATGAATGGACAATGGGAGTAAATCAGTGTATGGAAG
CCCCAGCTAGATAGGCACCGAGACGTAGGCTGCAAAGAAGACAAATGGGTGGAAGATGTCTCTATAAATATACATGGTTGTTAGGGCAAACTGA
CCATGCACATTGGNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNTCCCATTCCACATCAAATGTGTTCCATGGCTGCAATCAAGTCCATGCTCAAC
AGTACATGAAGATATGACAGTCTGGTTTCCATTTATATAATTGTTTTGTTGCCTTCTTTATTCTTACTGGATGAGGTCTTATTGTGCTGTCCTG
ATTGACCTGAACTCTGGGTTTCAACAGTCCTGATACCCTCAGCCTTCAGAGCAAGGTGCCTCCCAGTTCTGCCTTATTTTACTCTCTCTCTCTC
TCTCTCTCTCTCTCTCTCTCTCTCTCTCTCTCTCTCTCTTTACTAACTATGAATTCACACCAACAGTTTCTAGAATCTCTTTTAAAGCATACCT
GATAATGATGCTGATATTGGTTAAAATAATGTAACAAATGCTTCTTTTATTACTTTATATGAATTACCTGTCATTTAATGCTCTGAGATGGTGT
GGGGATATAGGGCGCTGTTATCGTTCCATTTTATAGATGAGAAAACTGAGTACACAGAGATAAAGTAACTCACTTATTTCCATAAAGCAAGTAA
GTGGTGCCACTAGAGTATAAAATCAAACAACATAGTTAGAAACACTATCCTTATCTGTCTTTAGTGTTTATTTCTTCACAGCTCCATCATCTTC
ATAGACACACACACACCCTACTTAAAACCTGCATGTCCATAATTTCCACAAGCAAGCACCTTGAGCTGAAACATTCGGGGCTGTTTTAAAGTAA
ATTTTGCTATTATTCCTTTGGCAGAATTTAAATTGTGAAGGAGATTTTCAAGTGATTTCCTCTCAGGCTTCATTTAGAGGCATTGAATAAAGTA
ATAGCACCTTTGAGAATGCACCTTGACATCTTAGTGTTGGGCACCTGTAATGATAGCAACGTGGGAGTCTGAAGCAGGAGGATTTCGAGTGGCC
AGGCAGGGGAAAGAGAGAGGGGGGAGAGAGGAAGAGAGAGAAGAAAGATTCCTACTCTGGCAGACTAACTGTTTCTTTAACTATCACTATTAAA
TACACTAAATTATTCTTCAGCTCCTCCTTCACTGTGGATCCATGCCTTTAAAACAAGCACCGTTGTCCCCTTTGCCTGTGCATCCTGAGTCTCT
ATACACCTTTAGCATTCTGACCCAGGATTCTCAATTGATTGCCACTAAAACCCCAGATATGGGTACAAATCTGAGCAAATTGATCTCAATATGC
ATTAGCATAATTAGAATTATCTAGGGATGTCAGATCATCTTAAGCACCTACAAAGTCCTATTTTGAACACTTAAGTATAATTAGCATTTGTGAA
AGGTCTCTGGTTTTCAAGTGTCTTGCAAGACTCTTGTTTTTTTTTTTTTTTTTTTAGTAAGCTTCTAAAAGCTGCTTGTCTTTAGGTGTTCAGT
GAACGGGAAAGATAAACATTTGAGGCTTGCCCTTTTCCTCTTTCTGACTAAATGCGGGTCATAAAGTAGAGCCACGGATACAGCAAGCCCCATC
ACACAGATGAACACAGATAGCCCTTGCCCTCATGGAAGGGATCATGTAGAGTCCTCTCTATTCCAAAAGAACTTCACAATCTCCATAACTACGT
TTGGCATTTCACATCTGTCTTGTGCTTTTGTGACATGTGGATTAGCAGACAGTGCCTCGGGCTCTCATGATGAATGTAGGGTAATACAGCAGCC
CAGAGCCATGGCACCTAAGTATGACTTGGATGTGATCCCACTTGGATATGATCCCTAAGGGACAGGATAAGGACACTAGATCAAATTTGGATCA
AAGAAGAATTGGAAATCAAAGAGTTGAGAATAACACCCAGCCCTGCATCTGCATCTGTTTAATTAGTCTTTTCTCCATCCATTTTAAATGTGTC
AAAAATGTGTAAAGATCCATTCAAATAGAAAAATAGTAATTTTTCATGGAGTGAGGGCAGGAAATAAATGTCAGATTCCCAGTAGTAAATAAAT
ATAATTTTTAGACATATAATTAGCTTTCCTCAATCTCAGTGTCTTCATCCTAAAAGGGCTTACTTTTGGTAAAGGGGAAATAAACCTGAATTGT
TCAGTTGGAGTCCAGTGAAAGAGCAGCAGGCAAAAGAACATCACAGCTGCTACAGATCCCACTTTCTCAGGAATGAACAGCTCCTTTGAATGGT
GTCTACGTTATCCAAAATGCCACAAAACAATTGATGATAAAACTGGTGGATGGACATATAGTCTGAATGTTTCTCAAAGCTCTTAGTACTCAAA
GAACTTAAGAAAAGGGGAATAAGGAGGTTTGCCTAGAGTAGAGTGTTTCGGGCAGAATAAAGAACCCCTTTATCTTCATTTATTAAGTTGAACA
GAACAAATAAGAAGCTATCCATCTCTATAAAAAATCAAATACAGTTAGTTCCTTAATGAGTTGCGTCTTTTGCTCCTAACTGTAATACATACAA
TAGTGTATTATTTCCTTTTACCCAGATAGATTTCCTGAAACCATAATATTCAGAGGCAGAGAACTAAAGGTTAATTTAAAAAAAAAAAACCCTC
AGGGGTAAATTATTACTATTAGAGGAGAAAGTTATATTTTTAAAGCTTATAGTATTATTTTTCCAAATTAATTTTTAAAATAGTATTTTCATGT
ATTCTTGAAATGTAAGTATATTCTAGATTACGTAAATAATTGATAATGGACTACCAAGAAAAAAGTATACATGTAATATTCAATATACAAATTA
GCTTCAGTAAATCTCTCTTTTGAGTCATTATTTCCACTAAAATGTCTCTTACATTGAAATTATTCTATGAAATATGAAATTTAGAATATAATGG
ACACCTTCTTTTGTTACTTAATCATGACGTAACGCTTTGTCTCCTTTTCATATTTGAAGTGCAGAAACGCCTTTAACATCCTAGGAAAGCCATG
TGGATGTCATGATCTGGCTTTCTTTGTTCGGATAATATACTAGATTATTTTCTTTCTTCTGAACATCTTAAACTCCTAGATGAGTTTTTCCTTC
AATTTTAAATGCTTTTGAAGCAGGACTCATTTTGTTTCGAGCATTTCTTGGAATTACATAGAGTTGAGTAAATATGAATATGCCAGCATCCTTC
TAAATGGACTACCTTTAAACTGTTTCAAAAGTAAACCATGCATCAAAAGTCAACTCGGAAGACCTGTGTTCTCTCATACCCCTACACCTACCCT
GCCCCCTGTGGGTTTTTTTTTTCTTGGTAATAACAATAAGTGTCTTAGTTAGGATTTCTATTGCTATAACAAGAACACTATGACCAAAAGCAAC
TTGGGAATGAAAGGGTAGCAATAAGATTACAGGTCTACATATCACAATATATCACTGTAGGAAGTCAGGGCAGGAACTCAAACAGTGCAGGAAC
CTAGAAGAATACGCTGATGCAGGAGCCATAGAGAAGTGCTGCTCAGTGGCATGCTCCCCATGGCTTGTTCTACCTGCCTTCTTAGAACACCTAG
GACCATCAGCCCAGCCATCACACCATCCACATGGGCTAGGCCCTCCCACATCAATCACTAATCAAGAAAATGCACTACACAAACTTGCTCATGA
GCCTAGCTCCTGTAGACATTTTCTCAATTGAGAATTCATCTTCCCAAATGAAAATGACTCTAGCTTGTGTCAGGCTGTTATAAAACTCACCAGC
ACATACAGCGACCCAAAACTTCATAGGTACTTTGAAATAAGTGAATCTCCAATCATGCTGCTATCCTTTTAAGAATTTGTAACTATCCCTGCAA
ACTGTTGTGGGCTATGTCATGGTCTCTAAATGCCACCCTCTTACATCCTAAGCAGAATGAAGCATGAAATAGGAACTAAGGTGTGGCACAATGC
AGACAACTTTCCTCTGGGCTCTCCTTACAGGACACTTCTCAGCCATGAATCTTACTGGCCTCTACCTTTCACATAACCAGGGAAACCAAAACAT
ACATCCATTTTTTAATCTTGTCCTCTTCCCCACACATTCTCAATCTTTTCTCACTGTTCCTCCCTCACAAAAGAGCAAGCATAGGAACAAATGA
GAAAGTTTCTGACTGCCATTGACTCTGGGAAAATATTAGTCTTGTGCTGATTGTTGCAGTGATGTACATATAACATGAAATTTACTATCTTAAT
CATTTCAAGTGCATGGTTAAGTACAATCACACCATTATGACAATCCCCAGTATATTTAAATTTTTATCACACAGAACTGAAACACCTTATCTCT
ATGCTACACTAACCAGTCCTTTCAAAAAATTCCAAGTCGTAAGTCTAAGATTAGGCAAGACATAGCCTTTGTGCTCTTAAACACTTTTCCAAAA
TATATTCATTTTAAATTTGTATGTATAAAAATGAATAGCTATTTTAGCATACAGAATTAAATCAGTCATTTTAATGATTCTACTGATTGGCTTC
CAGGAGCATCTGTGTGTCTTCTCATTAAAGTAACCAATGCATTACAACTGTCATTGCTCTGTGACTCTTCTTCTCTTGCTTTGCTATTTAATGT
TTCTTTGAAGTCAAAGAGTAGCTCTTCATGATATGCATCCAAGTGTGCACTTAAATCATAGAACTTATTCTTAGCAAGGAAGTATGTAGTGAAG
CACCTGGTCAAATGGGAAGACAGCCTTGAGAAAGACAATGTTTTTCCTGCTGAATTTCTAGGAATAGCCAACAAAGTGGGAGTCCTGATAGGTG
AGACCAGCAGTGTGCTCTTTCATAACCTCTTTCAATTACTCTCAACTCTTCTTTATTTTTGGATCCTATGTAAAATTTTCTATAAAGAAAAGAG
GTTATACTATGTTTTGAAACAAAACATCTTGGATCTCGAGGAACGTTTTGTCTCATTATTGAGATTCATTGGAAGCCTGTCATTGAATGAAAAG
ATATCATTGAAAACATGTGGCCTTTAAACCAACCACCGACTCTCTCCAAGCCTCTTTTCTTTATGACTAGAATGAGAATAACGGTGTATATCTG
CATCTTCTAACACCTGAGAAGATTTAAAAGCAGTGAGCAGTGTGCTGCCAGCCTCCTCACCCTGCTGCCTAACAAGGTCCCTCCCACATGAGCA
GAGCAATCCAGGAAGCAGGTGACAGAGGATGAGGTAGTCCAGAGCAGAGACAGGAATATAACAGAAACTGGATCTATCTGGCTCTGGATACTAG
ATCTAGGTTGGACGTAATGACATTGGGAGCTGTGACTGATGGGTTCTCAACCAGATGCCACCTTTCTCAGTGTCTAGAGGAGTGGTATCTAACA
ATGACTAAGATGGTTTCCCAAGAGAATTTGATACTGAAATCTCACTCCCTCTCCCTCTCCTTCTCCCTTTTCCATCCCTTTTCTTCTCCCTCTC
CCCATCATCCTCCTCCCTCTTCCTCCACCTTCCTTTCCATTCCTAGTGATCAAATTCAGAGCCATCCACATGTTCGAATATTAGTTAGGCGCGC
TTTCTAGCACTGCGGTACATCATCAGCCCTGGAAAGACTTATTGCCATGAATGAATCGTTACTGGCATGTACGTGATCAAGGCAGAGCCACCAT
GGTCCATGGGTTCTGTTAAATACCTCACTCTGAACATGACAAAACCTCGATATAACAACATTAACAGCTTAACACGTCACTCGTGCTGACCTTG
AGAAACTCTACTTTTAGAGACTTTCTGACATAGGTGCCTCTCCCCATCATTGATTTCCTCTTCGTCTGGTAACCTGGAAATTCTTCAGGTTACT
TTTGTTCTGGTTCTCTTCTTTGTTCCTTTGTCCCTCTCTTCATCCCTTCTTCATAACTTTTTCCTTTCACTGACTTCACTAAGACAAGATTCTT
TTGTTTTCTTCACTAACTAGTAAATTGCTTCTTTCCCAACTCTTACTCTTAAAAGCAACAGAATAGAGCTCTCTAAGGGAAAATAACATAAAGC
TTGAAACCACATAAAACTGCATTTGTATTTGTGTAGACATGCATGGATTGTTCTTTAGGCCAGTAGATTCAGAGACAAAAACAAACAAAAACCA
TCCCCCAGTGTTACAGATAGTTAGGGGGACTGGACTGTGTAACATCAATCCCAAGAAATACCTTAAGTAAGTACTCCCAGGTTCTTTAGAATGA
AAAATCTAGGAGGTGTCTTAGTTCTATTGCTGAGAAGAGACACTATAACCAAGGCAAGTCTTATTTAAAAAAAAAAAGCATTTAATTGGGGCTG
GCTCACAGTTTCAGAGGTTTAGTCCATCCATTATCATCATGACAGAAAGCACTCAGGCAGACACGATACTGGAGAAGTAACTCAGAGTTCTACA
TCTAGATCCTGCATGCAGCAGGGAGAGAGACACAGGACCTGGCTTGGGCTTTTAAAACGCCAAACCACACCCTCAGTGATTAATTTTCTCCAAC
CAGCCTATACCTACTCCAACCAGGCCACACCTACTCCAACAAGGCCACACCTACTCCAACAAAGCCACATGTCCTAATGCTACTCCCTAAGCAT
TCAAGCATATGATCCTATGGGAGATGTTCTTATCCAAACCATCAGAGATGGTATCAGATGCCCTATAAAGGAGTAGGGCTTTCAGGCCAAACTT
ACTTTGAAAGGTTCTCAAATGATTATGAGACACAACGGGTTTGGGCAACATAAACAGGTTGTTTTTATAAATTTCAACCACTGGCTTGCTATCT
TATCACTCAACCTTTTACCAATGAGCAGATTTCAACCAAAACAACATTTTTTTTTGATTAGTTGAGTGAAATCTCAATTCATTGCATCTCAATT
TATTGTCAGACTTTAAATCAAGTGGGCAATTATTTATTCCCATCAAGGCAGCCTTTTGCTATCCTGTAGCCAAAATAATAAATACTGGTTGTTA
GTTTTATATTCAACCTTGTTCCTCAGTGTTTTAAATTATTCTGATACAGCTTTGGATGGAGAGTGAATAAAAAGAGGCATGGTCTCTGGAGCCC
CAGATCTTCTCATAAAGACCCAAGAATAAAGTGGTCTAAACAACCCTTACACACTTGCTCACTCACTCAACAACTCTTTGGAAAACTCACCCAA
TCCCAGCTCCGTCTCTTGATTTACTCTTTATTCTCATGACTGAAGAACTCAGCTGTTTATGTTTTAATAAAAAATGGTTTAAAAGACCAAGCAG
AATCCAATGGGGACTGTTTCTGAGATATCCAGCTTACTGGCATATTCTAATGTCTACTTGGCAATAACTGAAAGTGTGGCCTTTGTATTCCAAG
TAACTCTGTCTTTGAGTAGTTTGCAGATTTATCACTTGTTGGTCTACAGCCTTTAAGTTAGGGATCATATCCAAAGCTGCCTGGTTTTATAGGA
AAAGCCTAGTTGTTTCCAAAAGCAAGGCCAATTAATAGAGCATTCGAGATATTTGAGATCTATTTCCATGGAGGAAATGATCTTAAAGTGAAGT
TAATTAGGAGTGCAGAGAGCCAGAGATGATAAATTCAGAATTGCATAATAGATTTGTATACTAAAGGGGCCTGTGTCATCTCCAGTCCTAAAGA
GACCGGCTGCTGAAGAATGAGACTCAGAAAAATGGGTGGGTTATATCGCCTCCAGCTTTCCATGTCACATGCACATGTCACATGGCCTTTCATG
ACCTATATTAAAGTTCTCCAGAGAAAGAGAACCAGTGGGTTATTCTGTAAATATGTGTGTAGGTTTATATATGCATGTATATATAAATGAAGGG
AGATTTTTTTATGGCAAGGGATTGAAGTGATGATATTAAGTTTAAATTCAATCTGGAGACCTAGGAATGACAATGATGTAACTCATTCTACATC
TCAACTGAGAGATTCCTGGTGGTATAACTTTTACTTAGAGGCCAAAACCTAAGGTACAGAGTAGAGACTGGGGGAGTCTAGTGACCTGAAAACC
AAGAGCTCTCATGTCCAGAGTCAGGACAAAATGAATCTCACATCCACCAGACTTTTAGTTTTTCTGTTCTGACTCTTAAAGGATTGGATAGTAC
TCACCTAAAACAACAACAAAAAAGAAAAAAGAAGAGAAAAGAAAACAGGAAAAACCATTCTCCTTTTGAGTCAAACTCTATTCTCTTCCAGTAA
TACTGTCAGAGATACATCAGAAATAGTATCTTACCAGCTATCAAAGTACCTTTTACCCTAGTCAAGCAGACTAGGCTACACAAAATTGAAGCCT
TTATGTCCCTGTCCATAGAAGACAGGCTTAGCTGGATAATACCCAAGTCCTCTATTATAGCATCTTGCCAGCTATCAAAGTGCCTCTTAGCCTA
TCAAGCAGTCTGGGCCATGCAAAAATCACGAAGCCTCAGTATCCCCATCTGCAGGAGACAGGCTTAGCTGAATAACACCTAAGTTCTCAATTAT
ATACCAGTGCTCTATGAATGCAAAAATAAGGCTTGGAACACTCTGCCTTGACCAGTTTAAGCATCAACAGAAGCTGATCGCTGGACATGCCTTC
AATCACAACTTACACATTAACTTATTAAATTTACTAGCTAATTCCAATTTGTTTTCTATTTTGAATGGCAAAAATTTTCAACTGGGTCTCTACT
TGATTTTATCAAGAAATTGATGGTTCTGTTTGGTATAAAAGTATTTTGGTTCTTTTTCCAGAATATGGTCATCATTACTAGAGATATAAATTGA
GCTAAATAGGAGTAAAGTATCATGTCTGTGATTTCATTTAAAAATAAAGATAAAATAAAATGTGGCTAGATCACATAAGATGCTGTTTGGGCTG
GCAACATTTCTCATTAAAAACTATATTCTGAAAAAAGGTATCAGGGACCTAGGGGAGACAGATATGGGTAACATTGACTGTTCTGGGCGTGGAG
GTTCAATTTATTTATTTGCCTTTTCACCCTCTGTAGTGTGTGTGTGAGTGAGTGTGTGTGTGTGTGTGTGTGTGTGTGTGTGTGTGTGTGTGTG
TGTGTGTTAGTGAAGTGGAATCAATTTCCTGAAGCCTTGAGAGATTTAAATCTCTTCACTGAGAGTGTCTCTTCAACTGCATGCAAGCATGAAT
GAATATTAAGGGTAAGGTACTATGACTGCCCTCTTGGTTCATCTTTCCATTGCTACTGCACAAGTCCCAGAATTTTGAATATCGTTGGGTCACC
ATTTGTCCTATAGGCTTCTGTAACCTCCTTAATCATTTGTATATAATCTAGTGTGTGTTCATTACACAGATTCAAAGCTTATGTTGGAGTGTAT
CTGTAGATAGATTGGGCTTTCTCATATTCTTCCCTCTACCCTCCTTGTTTTATATCCTCACAACATCTTTTCTTTTCCTTTTTTTGTTTGTTTC
TTGTTTGTACATATGAATTTAAAACTGTGAATTTATCTGTCTTTCTATATCTGGCTTGTTTTGCTTGACACCATGACCTGTGTTTCCATCCATT
TTCCTGAAAATGATAATACTTAAATTTTTATGGCTGAATTAAACTCCATTGTGGATTTATTCTATGCTTTTATCAATTTACCTATTGAAAAGTA
CTTATACTGCTTTTATAACTCGGCTATTCCTTAATCATTTTTCTAATTCTTTCTAATAGACATCCTATTAAATAAAACTGAACAAGGTATTGTA
CCAATGAACTCAGAACACATTTACTGGCTTTACATAGAACATTACAACTTTTCCTTTGGAAGGAAAATAGATGCTGCCCTCGAGTAGGGTGCAC
ACTATGTTCATATCAATTAACATTGGTGAAAACATTACAATTCAATTGGTCCATCAGTAAGTCAGAGTTGTCTATAGAACACATGTTTATAACC
CTATATCCCGCTCTGATAGAAATAACCGGTTAGCTTCTTAAGGTTGATATAGTAAAGTTGCCACAATTAGGTGGCTTAAAAAAACAGAAACTTA
TTTTCTCCAGGTTCCAGAGTCGAACAACTTAGAATCTAGGTTTATTTGGTACTGTGCTCCAATTGAAACCCTAGATAGAACCCATTCTTGTTTC
TCCTGAACTTTTCATGAGAACCAACTGCCTTGTCATTTCTCAGCTGAAAGCTGCATCCTCCAATCTTCATCTTCATATGTGATCTCTCCTGTGT
CTATGACTTATAAGCACACCATGAGATTAAGGGCACACCCTATTTCAGGATGACTTCATCTCTACTCATTACACTTTTTCCAAACAAGGCTGTG
TTCTGAACATAGAAGGGCTTCACCATCTCTTTTTGGAAGAAGCAACTCAAACCACGAGGCCATCCAAACAAATGACACTGTGAGATGGATTCCA
TTTGAATCTTTTATCATAGCTATAGTTCCTAATTTTACAAAAATGAAGGGCTAAAGTTTACTTCCTGCCATGCCTGGGAAATAATATTCAGACA
ACATGTTCCAGGACAATGTCTAGGATCTTTAGAAACACATACTCTGTGCTATATGGCTGTTCTACAGAAGCCACCTGAGAGTCACAATAGAGAC
ATCGGATGTAAGCTTCTTCTGAAAAGAATTAATGATGCTTCTATAGCTTGCCCTTGTTATACCACGCCCACTCCTACCTAAAGTCCTTCCACTC
CATCTGAATCACTGAAAGCTTTAAATCTAACTGTGTTGATGCTGCAGCAGGAAAGCCACAAACCAAATCAGGACAAACCAACAACCCAATCAGA
AGTATTTTATACAATTATTATGTTGGAGGTTGTGGTCTTTTCTTACTTGAGTTTTTCTGCAATGAGGGAGAAATAGTGAGGACCTTTTGTATCT
CTAGGTAAATATTAAATCTGCCAGAAGAGCCAGTTACCCAACAGGTAGTCAGCTCACAGGTCAGTGCTCATTGGAAGATGTTCAGAGCCAGAAA
CCAATCCCAGCAGGGATCCTATGCAAATCTAGCAAGACCTTGGCTTTCATCTTCTATTTCCTGAAACAAGCACCCAAGTGTTAACTGGCTTGTG
AAATACCTTGGGTAAGTCTCTTGTGTGTTTAAGATTCTGGCTGATTTACATCTGCCAAAAAGGGGTATTTGTTATTTCTTGATGAGGCCCAGGC
CCTCTGCACTAACATGTATGTTTTCGATTTTCATTCCCAACCTGATGCTTGACACTCTACATGCTTTCTAGGGTGAATGAAAGAAACTGAGAAT
CAGGTTTCTCTGTCATTAGCACAGAATCAGCTAAGAAAGAACTAGGATAAAGATGGAAGAAGGGGAAATGAATATCATAAGACTAAGGAAAAAC
CTTTAATATGAATTACAATGATTAAGGATTAGCATTAGAAAGTATGTGGGTTTGACAAAGCTACTGGAATCTTTTTAGACTTTAGAGCTCTCAC
CTTTCTTGAAGTCACTTGAGTTTGCCATCTTGAGATCTTGACTATTATGTAAAACCTCTAACTGTGTTAGGTCAAACAAGAGTGTAAGAGGTGT
GAGAAGGTTGCATGATTGCTTGTGACTCTTTTGTTGCCAGCTAATGTATGAGTGTGGAACCAGAGCAGAGAATAGAATGAATGGAACAATCACC
ATATTTGCTGAATAAATGTCTTTGTCCATTTTTTGTTGCCATTCCTGTACTTTATGAAGCAAATTAGTTCACTTCCTACAAGTCTGGACATGGG
AAGTCCAAAGCTGAAAGGCAGCATATGGTGAGAACTATCCCACTAGTAAAGATGCCCTGAGAACCCCCAAGCCCCAAGTCAGCACAGGACAGCA
TATGTCAGAACAACCACGCTGACTTGTGTGTCTCTTCCTCAGATTACGAATCCACTACTCATTATGCAGACATCCCTCTCATACCTCATCGACT
CTTAATTATTCCCAAAGGTCCAAGCTCCAATCAACACTAACAGGTGGTTTGTGGGTTATGAGAGGACAAACACGGGAAGAACCTAGTGAGGCAT
TTCAACCCCAAGGATGAAGTTGGCCATCACCCTGGTTTCTTCAATAAACTGAGCAAATGAGTAACAGTAAGAAAGGAATAGTACCCTGATGCCT
AAAGTCTCAACACCAGCATCTAGAAAGGAGTGCACACGACCCCACCCCTTATCTTCCTTGCCAGTTTCTGCTTTTTATTCAGTAACCATTGACC
CTGCCACCATTCATCACCAGAAGTCACAGAGTTGCAAGGTTATGTCCTCAGTCAACTCAGCTTTGATTTGTCCCCTTGGGTCCTCACTTAGTAA
AGAAAAGAGAACAAGGCAATACTGGTAATAACCAAAACTGAGGCAGATATGGTTTGGCTTGAGAGGTGGACTTGATAAGAGGAGAACCATTTAT
CACTTGGATGCCAACTTCTCATTTGTCAAATGTTTGATGGGTTTAGTCATGAAGTCTCCAGGCCAGGAGGACTACATGTTAAATAGCTTCTTAC
TTATTGTCATCTATTCAAAACAGGTCGGCTAAGCATGGACAATGGGTGTTACGATATCTTCAAGTAAGCTGTAGACTGACAGGCTAGTTCATTT
TTTACTATCTATTGTTATAAATTCATGGATTTTTGGGGGGACATTCTCATTGTGCCCATCAAATGCCTTGATCCCACTCATGCTCATGCTACTC
TGCTACCCACCGCAGCCTCTTTTCTTGGTCTGTATTGTCTTCCCTAATGCAGCCTTTTAGAAAATACCTTCATTGTGATAAAGTGTACATGCTT
AAAGCTCATGTTTAAAATGTATACTTCAGTGGGTTTTATGTAGATACAGAGTTATAAAACTGTCATAACAATCTAATTTTAGCACATATCACCA
AGTTAAAATGTGTATGTTAGCAGGCATAGCTCATCCCCCTACTCTCTCCACTCCCGTTAGCATACAATACTCAGTCTGTGGCAATCTCTAAGTC
ACTTTGCTTTGCTATTGCAATTTTTTCATAAGTTCCTACCACTGCCTAGATCATAAAATATTTCCCCTATATTTTTCTACAGTAAATTGAACAT
TGAAGTTACATATTAAAGTCTTGATCCATTTTGAATTGATTTTTTTTTTTTTTCGAGACAGGGTTTCTCTCTGTAGCCCTGGCTGTCCTGGAAC
TCACTCTGTAGACCAGGCTGGCCTTGAAATCCGCCTGCCTCTGCCTCCCAAGTGCTGGGATTAAAGGCATGCGCCACCACTGCCCTGTTTTGAA
TTGATTTTTTGCAAAATGGGAGATACAGATTTAATTTCATTCTTCACAATGTAAATACCAGTTTTGCCAGCACAATTTATTAGGAAGGCTGTCT
TTTTATTGCCAGCTTTGTCAAAGATTAGATGACTGGAGCTGTGTGGGTTGATCTCAGGTCCTTAATTTCACTGGTCTATAGGCTTGTTCAGTAC
TTTGCTATGATATCAATAGCAAAAGTAAATTCTATTAGTGTTTATGAAGAGTGAGTGTTTATGAAGAGTGAGTGTTTGCAATATTCCTTAATGG
TAACAGCATGAATAAGATTCCGATGTCTATATCTGTGGTTTTCAACTATCTTAATCGACGGCACTTAAATGATCCTTTGCTTCCCATTGTAATA
AAACATATCTAAAATGTACAGATGAGTTTGGTTTTGTTTATGTGGGATCCAACTCCTGACTTTGTGTGCTCAGCTGACGTGTCTGTAACGGAAT
GTTCTGCCAGTCTCCAGTGCTGTCTACCAGGACCAGCTAGCCACCTAATGCTGTCTGTAATTTTAACTTCATCTTCTCACAGGCTGAGTATAAA
TGAGGAGACACAGTAGGAGATATACGGAGCAAAGAGGCTTTTAAAGGTCTTAGGATTCTTCCAGAATCACTATTCAGGATGGTCTTCCTTAAGA
ATGGCAAAACCCTGCATGTTTATCATTTGTATGTGGACAGTTCAAAGTTCAGGAGGCCTCTTGTGGATGTTTCTTGTCTTCGGAGTATTAAATA
ATACTTAACTGTCTTCTGTTCTCATCATCGAATGAATATGACTTTGTTCTTATTTCCTAGGACTATGCTGTCAGTGATTATGAATAAGATCATT
AATGTGTATTCGTTACTTCAGTTTCTTGTGTATTGTGTACCAAAAAGAAAGAACAACAACCCAAAAAGTTGGTTAGAATTAGAGACTACTGCCT
TGTCATAAAGATGTACATGCTATATTTATACAACTTTCAGATGGAGCCCCTCTGTTGGCCCATGCTGACTACATATAACACATCAGGTTCGTCA
GCATCTTATTCCTGGGATACAACTACAACAACATGCTCAAAGAAAAAGAAACCTTGGTTTTTTTGTTTTTTGGGTTTCTTTCTTTTTTTCATAA
AAGTGATTGAGTGAGTTGTATCATTAAGCAATGATGTCACTAGAACTCATAAGGGGAAATCAGAAAGGCGAACCTGGAAACGTCTCGAAGTAAA
AACCAGGAACCCCCAGCACAGTGAGTGCACGGACTCATTGCTGTCAGCTGAGGAATTGCTCTTGTGTCCCTGCTACTGGAGCTCTGACTTGGTG
ATGTAAGATGGCCATCCCCCTCACCACTCATCCATCAGCAGCACTTTTAAATTTACCTTGGAAACAAAAGAGCAAACTGAAGTACAGTGCGTGT
CATATGATGAGTTCAACTACCTCTTCGAAAGGCAACATCAGAGAGTGAGATCAACAACTAGACTAAGAAGGAGAAGTCACTGTTGTTGACACAA
GCTATTAATTTACTGCTCTGAAATGATCGTTAACTGTTTTGCTCCAAGAGAGAGAAATCTGTTCAAGGGACAGAGAGAAATATTGTTTAGATGG
TCCCTAATGAGACAGAAGTAGTAGGGAAATAGAAATGGATTTCCCCACTAGCACTGCTCCCTAAACAGGCTCCCTTATTAACCAGCATAAAGGA
TGGAAACATTTTGGCTACATAACCCTACATAATCCTAACCCTTGGATACTAAGACGAGAAAGTATTTACTCTCTGATACTTCCAGGTAAAGAGG
GGATGTTCTCAGTTCAGATCACCAGAACCATAACGGTTCTCTTAGTTAATTAGAAGAGTATGTTCTCTCCTGGTGTACGGCATTTAGTGACTTC
CAACTTTTAAAAATGTAACTTTTTATAAACCTCCTCGCGAGGATTTAAAGAACAGGACACAGGTGACATTTAAGAATGATTTTAATACTGCAAT
CAATGAGGAATTAATTTTCTTCGAGGTGAGAGTTTCCTGCGTCTGGAAACGGAATCTAGTCCTTCACACCCACTAAGCTTGCACTGGAGCACAG
ACCTGTAGTCTCAGCCTCACATATTTAAAAAAAAAAAAAAACAATTCCGGATTTTATGTGGAGGTCCTTGATCCACTTGGACTTAAGCTTTGTA
CAAAGAAATAAGAATGGATTGATTTGCATTCTTCTACATGCTAACCACCAGTTGAAAATGCTATCTTTTTTTCCACTGGATAGTTTTAGCTTCT
TTGTCAAAGATCACGTGACCAAAGTGTGTGGGTTCATTTTGGGGTCTTCAATTCTATTCCATTGATCTACCTGCCTATCTCTGCACCAGTACCA
TGCAGTTTTTATCACTATTGCTCTGTAATACAGCTTAAGGTCAGGGATGGTGATTCCCCAGAAGTTTTTTTATTGTTGAGAATAGTTTTTGCTA
TCCTGGGTTTTTTGTTATTCCAATTAATTTGCAAATTGCTCTGTCTAACTCTATGAAGAATTGAGTTAGAATTTTAATGTGTGAGTCTCAGCCT
CACAAATTTTTTAAAAAACAATTCCTAGAGTTTTCTAGAATATATATATATATATTTATCACTTAGATGCTAGGTTTTTTTCTTTTTTTTTCTT
TTTTTTATTAGATATTTATTTCATTTACATTTCCAATGCTATCCCAAAAGTCCCCCACCCGCTCCCCCACCCACCCACTTCCACTTCTTGGCCC
TGGCAAGTCTGTTTTGTTCTTTCTGGTTTTTCTGCTCTCTTCATATTAGTAGCATGGCCCTGGTTTGCGAATGCTCCACACTTGACCTGAGAAG
TGGACTTACACTGGTCTCTTAGCTAAAATAAACAGAAAGCCATTCCGTTCGGGTCCACTGCTGGTGGTCACCTCATTACCATGCCCAAACCATG
GCCACTCCAGTTAAGATGATCACTGGCCAACTTCCAAAGAACCACATTTGTGCTGCAGTTATTCATTGGACTGGAACAACCTCCTCCCCGCCCC
ACCCCCACCCCCACCTCTCTCTCTCTCTCTCTCTCTCTACCTCTCTCTCTCTCTCTCTGATTTTAAATTGTTTGTATAGCTACTTTCTTGCTTA
CAAAATCCAACTCTGAATTGTAATATTCCATGTTGGTCAATCAAGGCTCCCTGTTTTGTTTGTTTGTTTGTTTGTTTTATATCATCAGTAGTAC
TCTCTATTTATTTTATATCATTCTCAAAAATATTTAAAGTAACTCGCTTCCAATATGTCTGTAATAACCAATACCAGAACTGTAAATAGGTTAT
GACAATAAGAAAACATATAACCCTTTTGGTATTTCCCTGTTACAGAGCACATACATCCCTCCATTAGAGTCACTACCCTTTGAGAATACCAACC
TTTCTAGATGCTCCTGGTAAAGCTAGCATGTGGGAAACATTGCTCAAAAATGCCAGGGTACCTCTGATAAAACACAGGCTGGTAAGACTTTCTT
GAGTAGCAGTGTGATAGTCTACCTGTGTTCTCAACTTTGGTTTCCAAAGAAATTCCCATCATAATCTAGTGAAACTCCTAGCAGTGGAGGGTAT
GGAGCCTGAAGTTGCCTCTTCCTCTAGCCAGGTAGAACTCCCAGTGAAGGGATTAGGATAACCCACCCACAAAACCTTGGGCCCAAAATTTATC
CTGCCTATAAGAAGTGCAGGGACAAAGATGGAGCAGAGACTGAGGGAATTCTCAACCAACGACTGGCTGGCTTGAAATCCATTCCGTGGGCAAA
AACCAATCCCTGATACTACTAATGGTCAGAACCTGTTATGCTTGCAGACAGGAACCTAGCATGGCTGTCCTCTGAAAGGCTCCACCTAGTGCTG
ACTGAAACGGATGCAGAGACCCAGAGCCAAAAGTTAGAACTGAGGAATCTTATGGAAGAGTTGGGGGAGAAACTAAGGGCCTCGAAGGGAATAG
GAACTCCACAGGAAGACAACAGAGTCAACTAACCTGGACCCTCAGGGGCTCCCAGAGAATTAATCACCAACCAATGAGCAAGCTTGGGTTGCAC
CTAGGTCCCCTGAACATCCTTAGCAGGTGTGCAGCTTGGTCTTCATGAGAGTCTTCCATCAACTAAAACAGGGGCTGTCTTTGTCTCTGTTGCC
TATCTGTTGATCCTGTTTCCCTAGCTGGCTGCCTTGTATGGCCTCATTGGAGGAGAGAATGTGCCTTGTCCTGAAATGGTTTGATGTGTCAGGT
GGGCCTCACTGTTCTCAGAGGAGAAGGGGACAGAGGAAGGAGGGGAGGAGCTGTGTGAGGAAGGTCAGGGAGAAAGGGCTGGGATCAGAATGTA
AAGGGAATAAATAAATAAATTAGTGGAAAAATGTTTATCAAGAATAGTGTACACAGCCACAATTAAAAAAAAAAAGAGCCATGAATTTAAAAAA
GCAATGGGGTTATGTGATAGGGATTGAAGGAAGGAAAAGGGAGGAAGAAATGTAATCATAGTATAATTATAAAATGCTCTAAAATTTATTTAAA
TGTAAAAAAAACAGTAGGGGAAATCAAAAGTGTCAAAAGCAGAAGGAGAGATGGGCATGTTGTCATGAATCCATACAAAGAATAGGAGACATAT
GAATTCAAGAATTTAGGTTGGATGTGAGTAGTTGGAAAGAACCATGAATTTTCCCCAGCAGATTATTGCAAAAATATCCAAACATTGGAGCAAA
AGTTCTAAGTGCAGATTAAATTGTAAGTTTCTCCAATATTCTAGAACCAGAGTATTCCAAATACTGCCTTCTTATGAGTTCTGTTTATCCAACC
CAAGTAAGATCCGTGCATCTTTGCAAAGTTCTTGTCACATCTATAGTGACAATGAGCATACTTTTATCAGAATGGCCCTTATCTTCCTGGGTTG
CCATAATGATTAGAAAGTTATTCTGCTCAGTTAAAAATTTATTCTAGTTCTGGCCAGTAACAGAAAAGTAGAAAGTCCAGCAAGATGTTTCATT
AAAGTTTACTCAACCCAAGTTGGCTGCCACATAATTTCCACTGGTTTGAGAAGACAGTAATTTGGGTGGATCCTTGAAAGCTCAAATTTTGGAT
AATTAATTCTCAGGGTGTCTGTGTTGGAAAGCAAAGGGTAGAGTCTAACAAGAAGCAACTAGGTCATCACTCATGAAAGCTTTCTTAGAAAAGC
AAGTTCTGCCTGCATTATTGTCCACTCTCTGGTGTGAGCCCTCTCCTCTTTCTTGTTTTCTCTGATATGCAGTAAGTTGAACTTCCTCTTTCTA
CCTTTGGGTGATAAAGCCATAGGGCTCTCACCAGCTGTGAGAAAGAGTATTTTATCCTTGGACTTCCCAGTCTCTAGAACTATAAAATATAAGG
CAGGGGGTCCACTCTGTTCTCTCAGGCCTTTGAATATATTTTCTTGGTGGCCATGGCCATTTTGAATGAACTCAGGACAACACATAGAGTTCTT
GAGAAGAACATGTTGTCTTCCTACCTTTGGCTCCCCCTTGTCTTCTATGTTTTCTTCATCTTTTCCTTTTCCTCTGACAAGTTGCATCTAGCTC
TCATAATTCTTTAATATAACATTTTAATTTTAAAAACTGTTTGGAAGAACTCATACATTAGTCCTGTATTTACATCTCCTTTCTCTCCCTCAAT
CCTTCCTCCATCTGAGGTCCATTACCTCTTCTTCTATAATTATAGTTACACAAAATTGTGTTTGTATACAACCTCCTGAGTTCGTTTAGTGTTT
CTTTTATGTACATATGTTTAGGTCTTGGTAGTTGGGATTGGATAACCTATTACAGAACACCTTGCTGGGGAAGTCTGACTTTCCCTACACAGCC
ATTGACTGTGTATAGTTCTTCATCTAGAGATGGAGCCTTGTGAAATTTTTCCTACCCACATTGGCATGTAGACTGACCTTGTCATTATCTAGAC
CTTATTTAGATAGCCACACTGTTGATGTTTCATGAGAGCAGCATCCCTGGCATGTCTAGAAGATACTATCTAGCAGCAGGTATTTTGGTCTTCT
GGCTCATACAATCATTCCATCTCATCTGCAATAATCAAACCTCAGGTCTAAAGATTGTATTGTAGATGTGCCAGTTGGGGCTGAACACCTCACA
GTCACTCATCACTGAATTTTGACCAACTGTGGATCCCTGTAATAGCCTCTTGGATGGCATCTTTGATGAGGGTTGAGAGCTAGCTACACTTGTC
TGTGAGTATAAAACAACACAAACCATTGTCATGACCCTTGGTTGCCTTGATTGCCTCCAAGAACTTGAAGGTAAGACCCTACTTCTTAAGTCCA
AACATACTTCAGATGAGGATATGGTGGCATCAAGCTGGGACTGACCTGAATGCCCCATTTCTGAGGACAAACTCTCATAGTACTAGAAGTAATA
TGCAAATTGCTAAGGAAGAAAAGAAATAAATATTCCTTCCCAGCTAGAAGGCCTATAAACCACAGCAATGACCAGCCTGCCAAGGGACCTACAG
TGGGGTAATAGTGAAACTATTATCTTGGAGTCATCAACAGCCATCTAATTAGACTTAAAGTCCACTTACTAGAAGGAAACTCATACCTGATACT
GGAAGCCTAATGAACACATGGCTAGACCTCATAAGAGAACCTACTATGATTTTTTTTCTAAATCAATAAATTATATAATTATTTTCTAAGTGAA
TAATTACTTTATTAGTTCTTTAAGAATATCAAACATGCACACCATATATCATGTCATTCCTCCCTCTTCCCCTTAACACTGTGGTTCTCAACCT
GAGGGTCAAATGACTCTTTCAAAGGGGTCACCTAAGACCACTGGAAAATGTGACTATTTACTGTATGAACCATAACAGAAGCAAAATTACAATT
GTTAAGTAACAACAGAAATAACTTTATGATTGGGGGTCACAACAACAGGAGGAATTGTTTTAAGGTTCACAGCATTAGGAAGGTTGAGACCCAC
TGTCTTAACCCTCACAGTTCTACTCATCCACTCCCCATCCACTTTCAACTTCAAGTTCACCTTTTACTTTTATAACCCAACTAATGCAATTTCT
GCCGCCCATGTACTCAAGGGGAGAGACCATACACTGAAGCATGCTCCACCTATCAAAGGCCACACCTTTAATAAAAACCGGCTGTCCTTGACCC
CGCAGCCAGAAACTATGAATAGCTCTTCAGGCTGTGATGGGAGTTCATGAACCCTGCTCTTTGCTGGAATGCTGACCAGCTGGATCTTAAAGCA
CATCTCTGAGCTGCTGGGAGCTCAGGAGTGGGGTGGTCATGTCTTCTCCAGAAGGCACTGTTTTTCTCTGGTCTTCCTTGACCTCTGGCCCTTA
CAATCCTTCTTTGCTGGTTCCTGGAACTGGGAGAAGCATATAGCTGCTGCTTTTAAAACTCTAAAAGGCAAGAAAATTCAGTTAAAAACAATGG
AGAACTTTGGTTGAAGCCAGGATAGGTTTATTTCTGCCTCTACAGAATCATAAAACATCATACACCTGACAGGAGTGGGAATGTGTAATTGAAG
ACGGCGAGTAACTATTTATCATAAGCTAAAATTTAATATAAAGCTACTCAATGCTTTAATATCCCATCCTAAAGGGCTAAGCACTATGTAGTGC
TAGATCCTTGAGTAGAGAACAATAAACAGAGAAGACAGTTGACGTACATGAGAATCCTCAGATATTACCCAAATACGTTTCAGATAATCAATAA
CTGTTGGCTGAGTGAATATGTACATGAACAAATGAGCAACCTACTAGAGCTCATTTTCCATGATGACTTTTTACAGTTTTTCTCCAATATGTTA
CGTACATTTTAATCATTTTTTAAAATTAACTCTCTATTAAGAAGCTTGGTACTTATGAAGATTAGACAAAATCGTGTGTTCAAGTGCTCATGCT
CAGAAATAGCGTCTGGCAGTCAACAAATGTTAACTAGTGATTTTCTTCTTTTTTCTAGTTATTCTTTAAAAATAGGATTATGCTGATGCCCTCA
GGCTCACTAATCTCAAAGTCATTTCCCTAATCCACTAATGAAAAGAGATCTGGAGTTGCAATCAGTTCCTACTCTGTGCCTTTAACTAGGCCAC
CCACACCAACCCCTTGGTGCTGGCACCAATAAAATATATGCAAAATCCTAAATATTTTTTCACTTCTTCCTCTGTACTGTTTAGTTAGCCCCAA
ACTTTGGGCCAAATCTACCATGTCCAGTCAGCACTTGTCTACTCCCTGTTGGACCTGATGTCTGAACTGAATTTACCTCATTTGATTCTGTTCT
TTCTCCTTCTCTCTTAATCAGCCCAGTGAAGGTTTTGTAATTTCCTTTTGGTTCCAACAAGGCTCACTCCATTAACCCAAGTTTTGGGATTTGC
CTCTGCCACATATGGATCAAAATTTCCAAACTGCAGTTGGCATGAGTGTTGAAACTTATCCCTCAAAACTAGGAGATAGGTCTGATGTTCTTTG
TGTTTCTGTATTTCAGGAACAGAAGGGGGGAAAAAGAATTTCACCAAGCTCAGATTTCCTTCTTTTATTTCTCTGTTAGTGCTCTATGTGACTG
GCTGCCCCACTGAATCTGTTGGCTCTGCCTCAGTCTGTGGTTCTCAGACCCAGCTACTCACCAAGATTATGCCACAAATGATTTCTTGGATGCA
TGTATGTGTTCTTCCTTCAAGGGCTTGTGTTTTCAGGTTGCTATGAGTGACTAGACCTGTTGCCAGAATGCATGAATCCTCTCTGATTTTCTGA
GTATATCCGAAAATTTATTGAGCTAATATCAATAGATAAGGCACAGCTTTGGTAAATTCATCAGTTACAATGACACCAGCAGCTCTGGATAGAC
AAGAAATACTTGCTGTCTTTTTCACCTCTGACTAGTGGGAGCTTGCCATGCAGAATCTCGTCTGTTAGTTACATAAAGACCAAAACTGTGAGCT
CTGTGCTCCTGAAAAGATAAAATTCTGGACTTGCTTGCTTCATGACACACTATGCAGACCGTGTCTCCTTTTATATTGAGAGGCAAAATATTGA
ACATAGGACTCCATGTGGAACCTAATAAAGGCACTAACAAAATTACACTTTCCACTCTGTGTTTTATGCCATACTCCTAACATTGTGGCCAAAC
TGGTCTTCAACGCTTTTACTCAATTTACTCAAGTGTGTGGGTGGGGGTGGGGGGGGTCATTGTTTCCTGAATGGTTGAATTTTAAAAACAAATT
TACTCAATGTATTTTATCATATTTCTCCCTCCCCATTCCTCCCAATAACCCTCTTTCCTAACCAACCTACTTCACTTTTTTCTTGCTTAAAAAA
AAAAAAAAAAAGGTACATTTTGTGTGGTCCACCTCTTCCTGAGCATGGGGCCTTCCCTGGACTGTGGTCGATATGCCCAGTGTCATTGCATTCA
TCGAAAAGAAGTGATGTTTCCTTTCCCTCAATGACTGTCATTTGCAAATATTGTTTTCGTGTGGGTGGTACACTGTACCCATTTTTCTTTCCCC
ATACACTGATTTTGTCCTGTTCGAACTTGAGGTCTTGTGCGTGCTAGCACCATCTCTGTGAATAGCCATGAGCATCAGTCCTGTTATGTCTGAG
AAACGCTGTTTCCTTGAAGTCGCCCACCACCTCTGGCTTTTAAATTCTGCCTCTACATCAATTCCTGAGCCTTGAGGAGAGAGGTGTTATGCAG
AGATTCCATTTAGGACTGAGTGCTTTAATATTTCTCACTTTCTGCAGGTTGTCCACTTGCGGGTCTCTGAGTTAATTGCCATCTACTGAAAGAA
GTTCTCTGATGAGAGTTCTGTAATACAGTGAGCTGTGGATATAACAGTCATTTATTTTGTTAGAGGAGTTATTCTGTTGCTATGTTCATTTAGG
CTTTCCCCTAGGCCCATCATCTAGTCACAGCTTTTGAACCTCATTAACAGTGCTGACATGTGTTCCATCTCATAGAGTAACCCAGAAACCTTAC
CAAACGAGTAGTTGGTTACGCCAATAGCACCTATGCTACTATTGCATCACTGAGTGTATCTTGAAGACAGATTGCTACTACAGCTCTCAGGGAA
CATAGCTGGATGAGACTTAAAACTGCTTTTCTTCTCCAGTAGCTTGTATTATATCATCACCCAGCACGATGAATGCTAGTCGTAATAGGTCAAA
CTTCTAGTTGAGTACCAGGTTGGTTTCTCCATGTTCAATACCATAAGTGTGTGGTGTCTTCAGTAATAATCTCTTAGCATCATATTGTGGTAGG
TACTCAATATCATTGGCAATACTTTGTAATATTTAAGGGTGGTCTGTGGAACTGTTTTTGCCAATGACTCAAAAAGATATAACCACTCCCAGGT
ATAGGTTTTATGGTAACATATGATGTCCAGTTGAGACATTGTCTCCCCCATCCTATGATAACTCCATTTAAATTCCATTTATATACGCATATAT
TTTAGGAAGCATCTACAGGAATAGGCTTCTGTATGGCTTTTCAAAAGACCTTTAGGGTAAGTTGTCCCTTCCCATGGTCCCTTCTCTACTCTGT
CCACCTATCACCCTCTCCATTTAATCCTCCTATTCTAATTTCCCTTTTATTGTGTGGTAACAATATAGTTATTTCCCATTCATGGTCCCTAGAA
GATCACCTCCTCCTTCATCCTAGCTCTCTACTTTCTGTTTAACCTTGGTTATTCTGATATTGAAACACATATATCTAAAAGCTAACACTGGCAT
ATGAGGGAAAACATTCAATATTTGTCTTTCTGAGTCTCAGTTCCATCACTCTGGGTGATTGTTTCTAGGGCCATCCATTTGCCTGCAGATTTAA
TAATTTCATTTTTCTTGAAAGCCAGATAAAATTTTATTGTATAAATGTACCTCATTTACATTATCCAATTTACCAGACGGTAGACTTCAAGGCC
TTTTCCAATTTCTGGCCATTATGAATAGAACAGCTATGAGCAGGGGGCCCTTTTGTATATGAACAAGAGTTTTATAGATGGATCTTGAGATAGA
TCAATTTTTGGCTTTTGAAGGAACTTCCGTACTGACTTCCATGGTGGCTATATAAATTTGCACTCCTTCCACAATGAATAAGTGTTCTTCTTTC
TCCACATCCTTGCCAGCATCTGCTGCCGTTTGTTTTATTTATCTTGCCCATCTTGACTGGAATAAGATGAATTCTCAAAGTAGTTTTTATCTGC
ATTTCTCTAGTGATTAAATATATTGGAAAATCTTGTTTCTCACTCAGTTGTGTATTATCTTTTGAGAACTCTACTTCTGTATCCCATGTTTTAA
TTGGGATATTTGCTTTATTAATGTTGAGGGTTTTTTTTTTTTTACTTCTTTGTATATTGTAGATACTTATCCTCTGTAAGATAGATAATTAGTA
AAGATTTTTTCCTATTCTGTAGCTGATGCTTTACTCAAATAATGTTGTCTAGCATGTAGAAACTTTTTAACTTCATGAGGTTCCACCTATTAAC
TGTTGGTTTTAAACCTTGTGCTATCCAGATCCAGGGCCAGAAAGTCTTTTCCTGTGTCAATGAGTTCAAGCCTATTCCTTAAGTTCTCTTCTGT
CATTTTCACTGTATTTTGTCTTATGTTGACGCCTTTGGTCCATTTGGAGCTGAGTTTTGTGCAGAGCAATAGATATAGATATAGGTCTGTTTTT
TTTTCTTCAGCATGTAGTTTGTATGCAGTTTGACTAGATGCTGTCTTTTTCCAGTGTGTGTTTTTAGGCTTCTTTGTAAAAAAAAAAAAATAAG
AAGAAAGAGGGAAAAGAAAAAGAAAAGCTTTTCATGGTGTGTATATTTATTCGTGCATTTTCAATGTCTCTGATTTTATGCCAATGCTATGCTA
TTTTTATTGCTGTAGTTCTGTAATTAATATGAAATCTAGAGAACCTACAAAAAATGAACATATATGTGCATGACATACCAAACTTAAATCAAGA
TTAATAAAACAACTTAAACAGACTCATAACCCTTAGTGAAATGCAAGAGTAATTAAAAATCTCCCACGTAAATGAACTCCAATGCCAAATGGAT
TCAGTGTAGAATTCTACCAGACCTTCAATGAAGAACTAACACCAATACTCCTCAAATTATTCCATGAAACAGAAACTGAGGGAAATTTCTCAAT
GCTGTTTCTATCAGGATCATACAGTCACAGATAAGCTTGTCTTTTAAGAGAGAGCAATGAATGAGACTATGCAGTGTTATTGTGGGCAGGGCTG
AGAAATAAGAAATCTGTCACAACTCATCAACGTGAAAAAGAAATGAACAAGAATGAATTTCTCTGGGGCTCAGTGAAAGGCTGAACCATGGAGG
AATAATATGGCTGGAGAGACAGACCACCACTAAAACTAAGTCACAGGAAAGGTAGTTGTGGAAGGAAAGAGGGCCCTTGCATGCCCATTTCCCA
TTCTCTACCTCATGCCCCATGCCTCTACCTCTGCCTCCATCTCCAGAAGGTTGATTCTTCCAGAAGTCACTCTTCTGAAAGGCAAGAGAGCCTA
GCAGTCAGAACCATCAAACCCAGAACAGAAAAAGAATAAGCATGGAATCAAGGATTGACCGGGATTAAGTGGCATGCCAGTCATCTGATACATA
TTGAATGAATAATAGAGGTGCAAATGATGAGAGCCCAAGACCCAGCATGCAGTAAAGGCTTTAGCGGCTGCTGAAACTACAATTCCCTTGCTGT
CTCTGTGCTCCGAGTCTCTCGTCAGTACTGTGTGGTATTTCAGACTGTCCACTTCAGTGCAGCCTCTGACCTGTGTCTTGTTGCCTCTGTGACT
TTCTCCACCCTCAGCTAATACCTCTTCATTTCCAACAAGAATAGAGAGTGTTGATGGGAACAGGGAGGAGCATGGGTTGTTCCGAACAGAGTTA
GGACATTATCAAAGAGAGCTCAAATAATTTCCAGAACCTAAAACACATTCTAGAATATTTAAGAGATTCTTTTTCTTTATCTTTTTTCTTTTTT
AAAGAAAATTTGGTGCCACTGCACCCTATGGAGAAAATTTGGTGACTTACTTCTAGGAAGCTTGCCCCAGACATCTGTGTCATAAGCTGTGAGT
TCACCAGACGCCTCGTTACCAAACGTCTTGTGGATGAACATCTTTTCTATTCAAATAGCTTTCATCCTATTATGGGCTTTGATTGAAGACACAA
TCCAGCTATCTAGTCAGAGCAGCAATGTTGACACTGCAGTGACTGTGCATGAGTGTTTCAGAGTTATGAGGACACTGGTGGCCACCATGGGCCT
CCTGCTGGTCACCTTCTCCTCCTTAGCTGCATTAGCAAGAACATCCAGCACTAGCTTCCTCAAGGGATCATTCCTAGTACTCTGGAATCTGAAT
GCTAAGAAATTATTTGAAAGCCTTTTTGGCTGGTTCCACTAGCCAGTTGGCTCTCAGATGGAATTGTGAAACTAAATTGGACAATAGCTATGAA
TTTATGTTCATAAATAAAAGACATTTTAGGGGAGGGGATGTCTTGCTTCCTTTTTTGCGTGTGTATGTGATCCATGTGCATATATGTGTATATG
GCCCTTGTTTATGCATGTAGAGGCTAAAGATTNNNNNNNNNNNNNNNNNNNNTTTAAGTAAATCCTCCTAATCTAGCTATATTTGGCTGGCCTA
GGTGGCCAGCTTGCTACAGGGGTCCTGTCTCTACAGCCCATGTTTGCTGGGATCTGGGATCATAGGTAGGCCACCATGGTTCCCCAGGATGTAC
ATGAGTGCTGGAGATCTGAAATCCAGTCTCACAGTCACATGGCAGGTGCTTTATCCACTAAACAGTCATCCTAGCCCTAAAAACCAGGTTTATA
AGGCAGTTTATGCCATATAGACACCTGGTTTGCATTCAAATGTTGAAAGAGTTCCTTTCTATCTAAGATCAGCATCTCCAACTCTTATCTACTT
TGCATCTCTTAAACCCTAACCAATGGGAAACAAGAGTCTGGAATTTCATCCAAAAGCCTAGAGTCAGAAATGCTAGATGGGATAAGATTGAGGC
TGTGACACCTGGTGTAAGTACATCCCAGAAGACAAAAGCCCTCATCCGACATTTTACTGAGTCTTCCACTTCTCACTCTAAAAAGCCTTCTCCT
GCCAAAGGATGTTTGAACCACTGATGAAGGATCTAACTTGAGCTTCACATAAAAGTGAGTCTCACTCAGACAAGTTCATATCTGTATTCCTCAG
TAAATCCCACCTAGGGCCTCCAATATATTCCTTATACTTTAATTCCAGTTGGGACAGCTGGTGAAAGTGATCTATGCACTATAGATTAGATACT
GAATTAATATTAATTTCATGACACACACACATATATACATATATATATTACATATATATGTCAATGGTTTTGTATACACACACACAAACACACA
CACACACACACACACATATATATGATGAATGCCCCATTCTTAGGAGAATAAACTCAAATAATTAGGGACAAAAAAGAGGCATCGTAACTATGAC
TTACTCTCTGATGACCTGGGGAAAATGTAATTTTTGTTACATAAAAGATATAGGATGGTGAATAACATATTAATAATTCGTGTGTTTGTGTGCA
ATACGTGGGATTTATTTTTACAACTTTTTTTTCCAAGATTGACATGACTTCATAATGAAGTTGCAAAAACAAGTAAGTCCAGCTTTTCTAATGC
TCATCTATGCTCTGCAACACAGGAAGCTAGAAATGCTCTCTCCTGATTTGTCCTGTTTGAGTTAAAATGAGTCATCTTCTCTTTCAGCTAAAGA
ACGCTTCTCTGTGGAGGTGAGCATCGCAGATGTTTTGCACATGAACCCTGAAGGTAAGGAGGGTTTTACCGAGTGCCCACATAATTGCCTCCCA
TTGTCCCTCTCATACAGACAAGGGCAGGGACGTGGCTATCTTGATTCTCAAAGAGCATCATCTCAAACCATCAATACTCACACAGAGCAGTCCA
CTATCCTCAAAAGGGGGGGGGGGATCTTCAATCTCTGTTTCAGAAAACCTGATGCTCGTCTCCTTTTCTTGTTTTTCTCAAAAGCTTTTCATGG
TTGAAATCCCCTAGCTGTGCAATTAGATTTTTATTTCCTCTTTATACTTGTCTGTACTTGGGAGGATTTTTTTGACAATGTCTGGGTTACTTTT
ATAATCAGAAAGAGAAAGATTGCATCTTCTTTTATTTTTTCAACCAGCTCAATACCAATTCTGCTATCACTTCCTGAGACATCTCTCGGCCTCT
TTAATATTGTCACTACTTGGGCCTGATCCGTGCCAGCCTATATGACAATACTTATATGTGGTGGGCTCTCAGTTTTCCTCACCTACTAATACCA
AGGGAGCCCTGGGAGCCAGAACCTGCCAGCAAATAGGTCATGGAGAAATCACCCACACAAAGATTGCCTCTGCATATACCACAGTCAAAGATCT
AGGCTACTTAAGAAGATAGCAGATTGCTTTTGGTCTTTGAGACCTTTCCTTCCTGCTCTTCAAGAGAAAGTGAAAAACTCATTAAGATAGAAAA
CTGACTATTATTATTAGCTGAAGAAAAATCTAAGTGAAACGTTCCTAAGATTTCTTTGAGCCACCTTGTGATGGATGAATAGTGATTCTCCTTT
AAGGGCCATCTTTCACTCCAGTGAAGGCTGGCCTGTCCCATGGATCAAAGATGCTCAAAGATTGTCAAGACCTTCTGAGTCAAAGATCTTTTTG
TCACAAAGACTGCTCTCTGGTAGTATATTCAGCAACATTGGAATAGAATACCACTCCAAACCCAGCTTCAACAACCAACAGTGTCTAGATGTTG
CTTAAGGTCCTGGGAAGGACAAAACCACCCACCCTGGGTAGTGACAGTAATGTGTTCAGACACTGCCTGATGTCTCCTAAGTGAAACAAAAATC
CCTTCTATCAAAAAAGCACTTCTCTAAGTTACCTAAGCCCTGCCAAGACCTTCTGTTTTTTTCATTTAAGTGGATTGAAATTCATAGTGTACAG
AGTGGAAACAGGAAATGTCTACAAAAAGAATGCCATAAACACTTTGGGTATAAGAAATCTCTGTGGCATTAATGTCATTTTCTAGACAATCCAT
TAACATAAGGACTGGTGCGTCAATCCAGCAAGAGTTCTATGCATTTCTTCCTACTTTCTTGGCTTCTAACCCTTGAGTTCACATGGTACTTGAA
GGATAGAGACAGATTGAGAGTCAAAACATTATGCTAAGTTCTTTAGTAATCTTTTGCCATGTGGATTAGGAAGCATAGACAACTGGAGTCTGAA
CTATGTTGCAAACACAAGAGAAATTTGCCTGCAAATTACTTTTTTTTTTACTTTTATGTGTACTTATTTATTAACCACATGTTTGTGTAAAAAC
GTTACCTCTGACTGACTTTTCAAATCTTTTAAACATCGAGGCATAGATCCTTAACTAAAATTCATCTTTTCCACTCCAAACAGTACACAGCAGG
TGAAGTTTTGGTAACAGAGAGATGTTCCAGTGGTTGGAACAGGAGATTGAGGCTCCAATATCACATCCCAGTACACACACATAGATATCTACTG
TGCTTGCTCATCTCTAAGTACCCTTGGGGCCTTTCATCCCTCTGGTTGACCTTCTTCCCTAGCATCTACCCTAGTCTCATGTCCTCCACCCTTT
ACCCAACCTGGGATGTGGTATGCAAAGATTATACTCATGCCAGTGGATATCTGTGAAAACACAGGCAGAAACTTGAAGGCAGTTAAGACAAGTC
AAGACACACAGCAACACAGCTCTTCTTCAACATGAGAAGAACATCTAGAAACAAATGTTNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNCTCCAGCTTTTC
AATTATGATCCACTAGTTGTAATCACTTCAGAGCATGGCAAAGCAAAGCACCGCCCTACCTGGCACCATACATCCTTTTACAAGAACTCTGGCA
TCGTCCAGGCTCTGCCCATTCCATGTCTTCTGCAGCTCCTGTCCATACACTTGTGACTTTAAATCTTGACAGATCTGTGAGATATAAAGACACA
GAATAATCCAAGAGCACTGTGGAGGAGTTGTCATTCACTAGGTGTCTTTTGATGCTCATCTAAAGGTCTTCACCCATTATCTTTGTATACATCA
CATGTTTCTGCCACCATCCAATTCTTGCCACTTGGGCTTCTACTTTGACTAAAATATGGTATCATTCAAAATCTGTGTGCTAATCTTGGCATAT
AGACATGTAGCTCGCTGATACATTTCTTGCTTAGTATGTGTGATACTCTGGGCTCACTCTCCAACAGAAGACAGAACAGAAAGACTTGGAGTGT
TGCCTATGAGGGTTAATACCTTAGCTTATGCATCATATTTGCTTATGAGTTGGACTTTCTTGTCCACCTGGGAAGACTTTGAACTTGGAACTCA
ATGGAGACTTAGTTTTGTTTTTGTTTTTGTTTTGTTTTGTTTTGTTTTGTAGTAGAGAAGCTGAGTGCACTGACCCTCATTCACTATGCCACTA
GCCCCAAATACCAATTTAAATATAAGCCTGTTGAAATTAAATATTAAAAATTCAGTTCCTCACTTATATTGAATCCACTCCCAATGTTCTATAG
CTGCACATGCTTAGTGGTCATGTTATTGGGCAGGGAATATCAAGAATACTTCCATTACAACATAAGTTTCTATAAGAACAAGCTGACCATCCCA
GTGAGGAGCTCTGGGAAAGTGATAAAGCTCAAACAGACAGGCTCAGCAGTGAGGGAGAGAGATGACAGAACAGGATTCTAAAAAGAAAGTGAAA
GGACCAGTCAACGTACTTCAACTAGGTCAACAATTATATCACTGATACCCAACCATGAACAAATGTACAACTACTCTACCACAAAACACTAGGA
AACAAAGAAGTAACCATCTTTGAGATGGTCTTTGAGACCCATTTTCTCCCCTGTCTAGTCTTTGGAGTCTGTGAACTCATTGTGAAACAAGAAA
CTTTTCTTCTGAATCCAGAGGCAAGAAGATCCTAAGCTGTGCTCACACATTGAAGCCAATGAACTCCCAGTCAATCGAGAAAGCAACACTCTGG
TAGCCATATACAAAAGCCTATTTAGAGCTAGGATTATTCAAACCAAAGAAAACAATACAAGGCAAAGGTACCATCTCTCTGGGGACTTTTGACT
CTATAACAAGTCCAAGGAACACAGGGGTTTCCAGGACAATTATTGTCCCTGTAATGGAGCAGATAGTGTGGTCATGGCCTCCTGCTGACAGCAG
TGGCTTATCCTTACCTAGCACATTGTCAACAAATAGATTACTTCCACAAAGTCACTCATCTTAGTCTCTCCTCACAGAGTCACCGAGGGAAGAA
GAGCAGATGTGGCCCTCTGTACTTTACATACAAAAGAGTGAAGGCACAGAAAGAGAATGAGACAAGCCATAGTTAGCAAGGTTGGTGAAATTCA
GGACCAACAATAGAAAACAGCCACCTAACCTGTCTGCAGTTCACAACTCCTCTCTCTGGAGATACAAGTCACTTAGCACCTGTTCAGGTCCCTA
AGTCTGTCATCTCCAAGGCTATGACCTCCTCATAGATAATGCTGCAAACTCAGCTTGTCACTCCATCCCTGTACAGAGTCCCCAGCAAGCTCAC
TGATGGTACCTCATCTACTCAAACTGCTGTAAGAGGGGACAGAAGCAATTGTTACTAATTCTGTTGCTGCCCTTCTCAGAAGACCCCTTCTTTC
AAGCAAAAACATACCTAGTAATTGCAGAAAAAAATCAGTGTTTGTCTCAGTGTATACACTAGGTCTACCGTCTATCTTCCTGGCAAAAGTCAGG
TGGAAAATGCTACCCCCCAAAATACTCTGTCTGCCTTGAGATACTCTTAATCATGGCAGATTTTATTTCTCTAGAAAACTGAGTTAGAATGTTT
CCATTCATATGACTTTCAATAGCCTGTAACCAAATATGATATATTCTCACACTTGAATGAACTCATCATTCATGATGTCTAATAGTCATTTGCA
TTCTTGGCTACATGAAAGATTGGATTCGATATTTTCCTTTAGCATGCAAAGATATGCAGAGTAGATATAGCCTTTATGTCTGATAAACTGTAAA
CACAAAATCCACCACTGACTAAAACAGTGAAGAGGAAGTAAGATCTAAGGCTGTTTAGATAAAATAATATTGAAGATGGCAACAAAAATTAAAA
TGCTCACTTTAAATGTTTCCCAACTAGCTGACACATCCAGCATTGTTTGGCTTGGAAACTTGACAAAGAGCAAAGAGTGAACGAATAAAGGGCA
GATACACATACAGAAAAGCCAAGTTAGCCAGGCTATGCACACTCTAAAAGAACAGCACTGTACTGGCTGGTTTTATGTGTCAACTTGACAGCTG
GAGTTATCACAGAGAAAGGAGCTTCAGTAGAGGAACTGCCTCCATGAGATCCAACTGTAAGGCATTTTAGTGATCAAGGGGGAAAGGCCCCTTG
TGGGTGGGACCATCTCTGGGCTTGTTAGTTTTGGGTAGTCTTGGTTCTATAAGAGAGCAGGCTGAGCAAGCCAGTAATGAACACCCCTCCATGG
CCTCTGCATCAGCTCCTGCTTCCTGACCTGCTTGAGTTCCAGCCCTGACTTCCTTGGTGACAAACAGCAGTATGGAAGTGTAAGCCGAATAAAC
CCTTTCCTCCCCAACTTGCTTCTTGGTCATGATATTTGTGCAGGAATAGAAACCCTGACTAAGACAGGCACCAACTCCACAGCACCCTGGAACC
TCAGCGTGTTGACTCTGTAGAGCACAGGAGGGAGAATGTGCCTAATCTCAGTAGGAAGTCTTACTAGCTATGCAATCTCAGGGCAAAAACTTCC
AGGAGGAGAAATTGTGTTGCTAGTTTTTGCCCACACTGGTCAGTTGGACTGCTTGAACACACTTTCAACATTTGCATTTGAACCAGAGGAAAGC
TTTACCATCCCTCTAAGGCTCCAACTAGGCGTGCAATCTTTGCCATTCCCATGCATCTGAGGAGTTGGTCTTTGACATGGCTGTAAACATGTCA
AACAATACATATTCACTCAGAATTTCATCTGTTCTTGACACACTCAAGACTTACGTGTGGCTCTTCATACCTAGCAATGCACCCCTTGCCTTCC
ATTACAGCAGAACCCCAGTGTTTCTCAGTCGGCATCAACCTTTCTCTTCATCTAAGCCATTACACGTTACAGTTAGACTACTGAGCTAGCTATG
TCAAGTTTTGCTATCTGCCCAAGGCAAATGGGCCACGCTCTGGAGATTCTACTTCCTCCCAGGGCTGAGTAAGTCCAGACTCTAAGCTTCTTCT
GCATCTTTTTGTGGCATGACCCCAGAGAGGATACTTGGAGCAGCAAGCCATAGCATGCCTGGCTAAAGGTGGATTAAACACTGGGCATGTAGCA
CTTTCCTAAGAAATTCTGGGAAAATCAGTTTTAGGTTTGATTTAGCAGGTCATGATAACTTCAAGGAACCAGCGTCAGGATGAACGGTAGAAGG
GGGGCTCCTCTCCCTCTTTCTTTCAGCAGAGAGGGAATTTTCTGCTATGCCTTCATCACTAGAATTTAGTGGGATGGCCATTCCTAAGGCTGCA
GTCTGGACAAAAAAAAAAAAAAGATTACAATGGCCCTGATTAGCATAGCCTGTTACTTCCTCATCCCCTGGGGCTGGATGCCTGATGCCTGACT
GGACTCTGCATAAAAACAAAGGGAGAAGTAGTTGTTGGAGAAGAAACTTATGGTATCTGCCAGGCTCATTGGGTGGATAGGACTGTGTCAAAGA
CAAGACAAGGACGTTTGTTCTCACCAGAGAGGCACCCTCCACAGGGAAGCAGCGTCATCTCCTCAACAAGGTCAGATCATTTATGACAATAGGC
ATGACACCTACAGAGCATTTTTTTCCTACACTTGTCATTTATGTGCTTTACTGGAGACTCTTTGAGAGATGGTATGTGTACACTAAAGGAGTTC
TTTCTGACAAAATTTATTTTATGTCCTGTTCTTACATCTAACTATACATAGCTAGATGAAAAGATGGGTGGATGAATGCATGGATGGATGAGTG
AGTGGGTGANNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNATGGATGGATGGATGGTTGGGTGGATGGGTGGGTGGATGGGTGG
ATGGATGGGTGGATGGCTGGCTGGGTGGATGGCTGGGTGGATGGCTGGGTGAATGGCTGGGTGGATGGCTGGGTGGATGAGTTGATGAGTAGAT
GGGTGGTTGGATGGGTGTATGGATGGGTGGATGGATGGATGGATGACTGGATGGGTGGATTGATGGGTGTGTGGATGGGTAGGTGGATGGATGA
ATAGTTGAACATTTAAATCACTAACATGGGTCAATCTACCTGTTTTCTAACCTGCCCCACTACACTCACAGCTGGATCTTTATATATTCACTCC
TTTCCATTCCCACCCAACAAGTCTACAATGAAAGTATTCACTCTATTCCCTGAACTAGCCTTGGGTCTGGTTGTTACCTCTCTGACTGGTTGGT
ACCTTTTCTGCCCAGAAGGTTGCTTCTGAAGAAGAACAAGATGGAAAAGGGAGAAGGGGAAAGCAAGTAATTATCTTGGTAGATATAGTTGCCC
AGATGTTTGAATGCAGAAGTAAGTCTGAAAGAACATCTTGTTCATGAGTGATAAAGTGTGACTCAGAAAAGTAGAGACAGGTTGTGCTCCTAAG
GGGAGCACAGAGAGGAGGGTTGACTTTGGGACTGGTTCTGAGCATTATGAGGTGGGAGAGATGTGTAGGCTTTCTGTGTTCCTGGCCCAGTCTG
TGAACCTACAATTACTACTATCCTTTAGACTTCCAGCTCCTAGTGCTTTGTATTACAAGTCTGTTGAGCCAGAGGAAAATTTACACAGAGAATC
TCACACCAGGAGCCCTGCTGAAGTTTCTTCTGAATAAGATGGCCTGGTCCTTCTCGGGGCCCCTGAAGAAGATATTTTCTGCAAAACTGCCATT
CACCTACTTGTCAGAAGCTGAATGGCAAATAATGGGTTCCTGGATGTTTTAAATGCACTCAGACACAGTAATGCAATTAGGGAAAGTAGGAGGA
AGTCTCGAAAACAACTGAGCAGCCAGTGGGCATGAGAGCTGTTGAAATCCTTCACTCTAGCTTGAGAGATAGGTACATGTTCAGCTACAAAAAT
GGTTCATGGAGGTTGGTTCTCAGCCACTGCTTTACTCATTGCCAGAGACACAGAGGTCCGATACTCTATGGTAGCCTCTGTTCACACCACTGAA
TGTCAGGTTTTCCTTAGCGAGCTGGTTTTGTTGCATTAATGATGGTGTTCAAATCTGCCTCCTCAGTTGCAATAGCAGTCACTTTTTATTTCGT
TACATTAGAAAAGTAGTCATTACCCACTTTAGAATAATATTCCCTCCCAGAGGGATTCACCATTTCTGTGTTAGCATGAATCTGTTTCGTATCC
ACTGTCTGAGCACCCGTGCAAAGACATTTGTGAGACATTAGGATCAGGCATGTCTACTTCCTGTATCTAATTATTCCTGCACTTCACCTCTGGA
AGTCATTGAAATTTTAATCACATTATGTCAAGGCTTAAATAATATTCCACTGTTGTCTATGCTATGATCTGGTTAAGCAGCCCTTTAGGATCTG
GTGTTTAGATTGTTTTCTAATAATTTGGGCTTTATAAATAACACTACAGAGGTGGACTCTCTTTATTCTAATGTTTTGTGCACCTAGGTTCACT
CAGGTTAAAGTTCTGAAGTGTATTTTCTGATTCAATGTGTATCCGTATTCTTGAGACTTAACATATGAGAAAAGTTAAACCAATGTACACTCAA
ACCACAAATCCAAATGTATTTATACACTCAGTAATACCATTCTGAAACCTCCAGGGTTGAAAAAAAAAAAAAAAAAAAAAAAAAAAGAGTAAAA
ATAAAATATTTTATATGAAAATATTTTGAAAATTAAAATTCCATTAAAATAAACATTGTCATACTATCTATTTTTAAATGACACAAGTAGAAGA
TGTCAGCTTCTTTGATGAAATATTGTGGTGTTAATAGCCTTTTTAAAGTCCCAAATCCTTGCTAACTTACCTCTCTACATTCGGCAGACACTCA
GACACTCAACACAGGTTATTTCCTCAGAACCATTTACTGAATACCCATAGAGTATAGAGCTTTGTTTTAAATTTGAGGATTAACACACACATAC
ACAAAGAATTAATCAATGAGCTTCTAGTCTAGCCTGAAGAGCTACTAGAGTGGGGAATTGCATAAAGATAAGTCAAAATCATTATCAGGACTAC
CCTGTACAACAGCTTTGTGAGACCTAAAGTGTCTGCTGGATGGGAGATGGGACAGAGAATCAGGAGGTATCTGGTCTTTCCATTGGATGACCAT
GAACCTATCTTTGCTGCTTCTAAAAATACTCATATTTTCTAGTAGCCAGAGTTCTGGATGGAAAGAGAGACATCATTTTGGGCAAAGACTTATT
TTAACAGTCTATATGGTAGGATTAATAAGATCCCAATGTTAAGTATCATCTACCATTGCTCTAGAAATCAATAGAGCTTTGTGGTGAATTAAAT
ACAAAAGGAGGGGCTGACTATACAACTGTTTGGAACCATAGTAGGACGCAGAGATTTTAGGGAAGTGTGAGGGAAAATAAGGAGGGGGGGGGGC
GCAAATATTGCCACTGTTGGAAGAACAAGACAAGGAATTTTGCCAAGTATCTAACGTGTGGTGGCCAGAGAACCCTTCAGCTTTGTCAAACTAC
AAAATCCTTGCTCTTTTTAATGCTTGGAGTGACAAGGGTCTGAGTGTAGTTTCCAGATGGGTGCCTGGTGAGAACGAAGGCCACCTACTGGAAG
CTGTGACACTTCAATACTCACCCTTCATTCTGTTCATGTGTAAGCTCTACCATGGACTCAGAACAGTGGTGCTGAAGCTGGAAACCCTTCCCTT
GAACTGTGCAGGGTATAAACTGCCCATGACCTGCTTCTCTTCTCATTTTAATCTGATGGATACCCAGGTTCCCATGTGCTTGGCACATGAGCCT
ACAGTTTTAAAGAACAAGTGGGGAGTGTAATGCCATCTTTTAGACCATAAAGGGCATGCAGAGGTTTTTTCCCAAACACCCAGGTATGTGTGAG
CCCTGCCACTTAAGGACACCTAAGAAAAGCATCTCAGGCTATTATAGTTTCAAATAAATTGACCATTGATAGAACTAAGAGTATCATCAAAGCG
ACCAATTTTTCTCCCAAATCACATTGGTTTGATAATATTAAATCAATTTCTGTTTCTAAAACTCAGCTGACAGCCTTTGGACTACACTGGTGCC
TGCCAGGCTATGGACTGAAATCCTTATGGGTGATTCCCAAGTAGAGTCTTTGTGGAAAACAGGAAAATTGAACTAACCTTTGAGTATTGACATC
CCAAACTATTATCCCTTTCATAGTTTCAGAGAGTAGTCGTTATAAGGCATGGTGCTGATTAGATGTTATGGAAACAGGCAGTCATTTTATTCTT
ATAAAGCAAAGAGTAACAATGATTTGCTGGGGATTGCCTGGTCATTGTTATGGTAGAAAAGTGTGTAGATGTTTACCTAGGCCTGTTCTACTTC
ATTGACCTGGGAAAACTACTAGTCAAATGGAAAGTGGGGCCCCGATTTGCTTGATAAATAGCAGTGTAATGGAGGACCTAAGAAATACAGAGAT
ACAGAGAAGTATTCCTTCCATAACTTAGAGCCACACTTGAGTTTTACGACATTAAGCATGCTCTGTTTTTTTTCTAATTGATAGTTTCTTTTGA
CATGTTTTTATTTACTCTTTGAATTTTTCATATAATATATTTTGATCACATTCCCTCTCCCATATCCTCCCAACCAATCTATCTATCTATATAT
CTATCTATCTATCTACCTATCTACCTATCTACCTATCTATCCATCTATCCATCTATCTCTATCTCTATCTCTATCTCTATCTCTCTCAAAATAA
AAAATAAAAAAACAAAAATCAAATCCAGTTTTCCTTGGCCAACAACTCTTTGACAGGGGACCTGTTTTGTTGCTTTTACCTTACACCACTACTT
AAAGCTCAGCTAAGCAGCTCAACTCTCTGGAACACAAATGAGATCCAGAAAGCAAACCAAATAGACATTTTGATGAAGTACATGTAATGAAAGC
CATTTATCTATAAGACAGGTGGAAAATTCCTTTGGTCCTCCCTCACAACTTATTGTCATCATTTAAATGACTTCAACTTTCTAAATTTTTATTT
TCATGTTAGCTTTCCAACTTTGCAGATCAAAAGCAGCGATGGAATGAAATGGATCTGTTTAGGCCTACTAGAAGAGAGTTAGTAGCAAAAACAG
CTATTAACTATTAAATTAAAATTTTTAGGGAGCAAAAACAATGAAGGATAGAAAGGTCGGGAGAAGAAAAAAAGACAGCTAACAAGAAAATAGC
TAGGCACTGAGGATCCTTGTGTCCTAGGTATCTGATTTAAGGGTCTTTGGTCTGTATTTACTCTCGGTATAGCATCCTGTGACCATGCCACTCA
CTATAACTGATATGTTCACAAATGATATCAGTGTTCTGATTAAGAAGTCATAGAAGTAAATAATATTTACATTTGTATCCAATTCATTAAAATA
TTTTTTATCTGAGAACATGCTTTACATAGCTAGATACATAGCTACCCTGTGATGTGTTTTCATTGAAAAATACACATTTTTTAAAAATCATTTT
ATTTCATTTGAAGATTGTACACTCGAAGTTTTGTTTGCTGCTGAAATTCCACACATTCCTTCAATTTTCAATAATTATATTTCTTATGTAATTT
GGCTATTAGCCAGATGCCCTGGAGCCAAATCTCATTGTATGTTTTAGTACATCTGTTGGCGCCAAATCTCATTGTATGTTTTAGTACATTTGTT
GCTGAGTTCAGTTTAATGCACATCAAATGGTATTGGAGGAATGCACAAGACCTTAGTCACTTGAAGTGCATCTTAATAGGAAGAAAGAATTCCC
TGATTCAAGGGTACATGTTATTGCTCCTCTCCTTACCCCCGTGGATCTGAGACTGTGAGAAGGGGTGAAAATATTATGTTCTTTATGGTTTTCT
ATAAGTGAGTCAGTCCGAGTTTGTAACTTAGTGGTAGATTGCTCACTTCAAAATCCTGACGCTCTAGGTTCAAACCCTGGCACTACAAAGAAGG
GAGAAAATGGTAAAGCAACCAGTCATTCAGCAAGTGTTACTGCACCGGTGAATGTGTGCAAAGTGACAACGCGAATGGTATAGAAGCTGAACAA
AATGACTTTGATAATAGTGTTCACAGTGACACTGAATCAGCACACCAGACATCAAAGGTTTACTTACTCATAGGTACTACTTGATTGAATACTG
TAGTTATCACCCAAAGTAAGCAGTAATAGCCAGCCATTTCCAGATTATTCATAGATTAGTCAGGCTAAGTAAAACCAAGCATTTCCGATACTCA
GTCTCTAGCTACAAAGCTTACTAACTGGATTGTTACTTCTCAGCCCTATAAAGTTCAGTAGGTTCTGAGTGGAATTCTAGAGCTTCCCTTCTGT
TCCATGATCTCTTGGGTCCACAAACTACTACAGGCCAGGAGAGAGAAAGGAATGTGCAGACAAGGATGTGGAGGGGCAGAGTGTATTTATGGAA
AGAGCTTTAAAGGTCAAGACTAGACCAAAAAAAAAAAAAAAAATGTAGCCTTTCTCCCCATTCTCCAATGCCACAGCTCAATCATCTGACCCCA
TGTAACCATAAAAGGAACAGTTAGAAGAACTTTAGCTGTAGAGAGAAAGGAAGAATAGACCCAAACTGTGGCAGGTTCTGGATCCTTTTCCTTC
TCTTGTGAGGGACTCTCAATATCCTCACTGAAGTGGTAAAGAAAAGTACAACTTAGAAAGAAGAGATAATTTTGACATATTCTTGGGGTGAATT
TCTTGTTGGACTTACTAGTTTTCTCAGATGTAGGTACAGCTCATCCTCAGTGCTCTAAAGATATTCCCATCAGACACCTGGCTATTTTCCCATT
GATGCTCAAAGGAGGCCATGGGTTACTCAAGGCCACACAGTCACTGTGAAAAGGGAAGAACTGGACCTCCCTCACACTCCTCAGGCTTAGAGAT
GAACCTGGACCAGATTTTCTCAAGTCTTTGGTTTCCTTTCAGTGCATTTTGGAGTTTTTGGATTCAAAAACTCCAAAAATCTCAAACAGGCAGC
CAGGGAGTCAGTAAACCGATAATTAATCTTTTTTATTTTCGTTTTCAATTTTCCATTTACTTCCAAGCTTGTTTCAAGAAATTAAGAAACACAC
AAAAATAGATAAGCTTGTTACCAACAGTAATTTGTGTGTGCTCATGTTTTAAAAGGAATCTAAGGTTCTTTTCTGTTTAATTAAACCGCATGCA
CACATACACATACAGCTCTGTAAAACAGAATTTTCTTCTAACAATCTGGCAAAACATATGTCTCCTCCTACTACAGTGGCCTCTTGGTAGTGAC
AGCTTGCCATAGAAACAAACTACAGTGAGATTTCTGTGACTTGAAACAACATTCATTTTTTTAAAAAAACTCTTTTTATAAGAGGCAGCAGGCT
GACTCTGGTGCTGCTGAGATTATCTGGGCTCCTCTGGGCTCTGGAGAACTTTTTGCCTTCTTGTTTTGCCACCCAATTGAGGATTTATTATCTT
ATACTAAATAGCTCCATAACTTAGAAAAGAAAACCTCAGCCACGTCCTGTGACTGTTTCTGGATGGATGGATGCAGCCAAAGTATCACCCAGTC
ACCACCACCTTTCAAATGGATGAGTCTAAAGTAACTGGATTGCAGTGTGTTCCCTTTCCAACATAATGACTCAGTATGCCTTATAATTATACAT
GCTTCCCCATGACTCGGAATAAGAAAATATTTGGGGACAATAATCCACTTAGCCTGTCCCACCACCACCTGACCTTCATTCATCGAAGTCTCAA
ATGTGGCATTTCTCAGAGAAAGCAGCCTCTCTTTCTCTTTTTCTCTGTTTACCTCTAAATGGAGCTCTCTAAATTCAGCTCCAATGTCAGTCAC
AGAATTACATTTTTCAAATGTCCCTTCCTGGGCCCTACATCCAGAGACATATGACATTAGGAGATAGTGTATACAGAAAAGTCTCTAAAGACAA
GGCCCAGGGAATGTCTACTACTATAAAAGAAGAACTACTATTGGCCTCACACAGTACCCAGCATTAGAGCCACCTTTGGGAAGTTGGTTTGAGC
ATAGAGACCCTTATCAAAACAAGGGAAAACAGCCTTGGAGAAGTCACAGTCTGTGGTAGCAAGGTCACCTTACCTGTTCCCTGCTCTCATACCA
GGTACATTTTGTATGTCTAAGATGGGGCTCCTTAAAGGTAGGATCAGTGGTCCTCTGTAGATATATAAAGTAGAAAGCATGCGATAAATATACA
AATTCTGGTGTTCAACTCCTAAGATCTTCTTCACCCACTAGCTATGTGATCTTGGGCAGTTCATAGAACATGTCTGAGCTGCAGCACCCTTATC
TGTGAGATGAGAATAATAAGGGAGATAGGGTCCTAGTGGCCTTTAATTAATAAAGATACTGAGGAAGCCATATAAAGCACAAAGATTACAAGTT
CAAAGCTTGCCTTGGACACAGGGTAAGTTGGAGATCAACATAGTCAACTTGGCAGGACTATCTCAAATATAGTTGGAAAGTGGAGTGGAGTTAT
ACCTAGTAATAAAGGGCTTTCTTAGCATGTGAAGCCCTAGGTTAAAACCTTACTACCAGGGGGAGGAGGAAGAGAAAAAGAAGAAGGGGAAGGT
AGGGGAAGAGGTATAGGGAGAGAGAGGAGGAGCAGAAAAAAGAAGAGGAAGAGACAGAAAAGGAGGAAGGTAGGGTTGGGTTCAGACTCAACCC
CACTCTTGTATATTATGTATACATAACCTATACACATCTTCCTGTACACTTTAAATCATTCCAAGATTATTTATGATGCCTAATATAAGCTAAA
TGATACATTCAGTTATCATACTACCCTATTTAGAGAATGACAAAGAGTCATATTTGGTTACAATGCAAATGCTTTCCAAATGCATTCAGTCTGG
AGTTGATGAAACTACAGATGCAGAAGCTGTGGATGTGGAGCGCCCAACCATCTTCCGAAGCTCAGGGGACACAGTATAGGAATGGAAACTCTCC
CACACTGGGAGTGTGAATCGTGGTTTGAAAGCCACTGCAGCCTGTGGAGCTAGCTGTGAAACATTCTATCCAAAACATGTTGCCTTTGCACTGT
TATTTATTGATGGATGATCTCATGAGTAACTGGTTCCTCTGGACCTCAGGCCAACATGGATTTTCTAACATCTGTCCAAACTCTGCCCTGGACA
GGGAGAATTAACATTTACCCAATTCCTATTCTTTCCTTTAAGCCAAAAAAAAAAAGCTGATTAAAAACAAAGCTCTGCAGTAAAAAGAGGCAGC
AGGGACTCTAAGTTGAGTGAGTTAGTGTAATAGGAGATGGGTGTGATGCCTCCACCTTTGGGTAAGCATCTACATGTTAAAATCACCCTAGTGC
AGAAGTGAATACAGCACTGAACTGTAATTTGCTACATCTAGCTTTGAAGATAGAACTCCCTGGGACACTACTCTGATGTTGGACCAACCTGTGC
CAGCTCCCATTTGGACCGCACTGTCTCTGCTAGTTAACTCTACATCTTACAGAATGCAGCTGTCTGCATACACAGCACATGGCCGTACACAGCA
CAGCTTCCTCTCAGGAACCTCTTCAGTTTTTGAAACAGGAAGAGAATGGGGTATGCAAACTCAGAAGTGAATGTTTTAAAGTGTCTCCATAGCT
ATTGCTAGTAGCTTTCACACAACCATGATGATCTAGCTAAACCACTGAGCAGTTATCCAAATGGCAGCCAACTTAGGATGGTGCACAGGGAGGA
CAGATCTTCTGGAAATTTTAATTGAGGATAAATTACCACCCTGTGGTGTAAGCATCTTTGCGCTAGATTCATTTTAATAAAAGAAATGCAGATC
CCTTATTCCTAGCTGGAGTTATTGCTCTGAAAGCCAACGTACAACAATCTTAATCTTCAGCATGGTGTGAAACAGCTAATCCCTTCTGTTCTTC
TCTTCCCCCTGGGAAGATTATGTCTTTTGTTTTCAAAATTAAATGCTAGCATTCTGTATTGGAAAGAAAAGTCAGACTCCTGAGAATCAAGGGG
CACATGTGAGGTTCTTACCCAGTTATTTCACCCTCTGGCCATTTAGTACTGGGGGCAATTGAGGTGATCCAGTGAGCAGAGGAGCCCAAAGGAT
GTACCTAGCAGTGCATAACACTTAATAGATAAATTTATTTAATATTAGTTTACAAATTATGGAGGCTTTTTTACATTTGGGAGACAGAAATGTT
AGAAATGGATTTAGGTTAGTGGAAGAATGTATGGCTGGCAGTACACTCAGTGCTTATAGTAAAGAACCATTGTTGGTGAGCTTTATGTGTGCAG
GGCTTTCTTATGAGGGTCTTGAGTGCTACCAGAGGGAGAGTCTGGTTTTTACCCTGATAAGTTCTCTGTTCTCTGCCACATTGCCCTACAACGT
TAAATGTACAGGTAGCAAGCGCTAAGTGGATTCAGTGAGTTTGGTTTGGTTTGGTTTGGTTTGGTTTGGTTTGGTTTGGTTTGGTTTGGTTTGG
TTTGGTTTGGTTTGGTTTGGTTTTTAAAAAAGCACATGAATTGAAAGGAATAGTGTTAGGGAGGGTTGGTGATAAATTGGTGGGGAGGAAAAGG
GGGCATCTTTGATCAAAATGTATTGTATATGTATATGAAATTCTCAAACAGCTTTTAAAGTTGTCCAAAAACCAAAAACAACCCTAACAGTAGA
ATTAATGTAGAAATTACAGTATCACATTCAGCAAAATATAAATGAAATAAAACAAAAATAAATTATGTGAAACTACATACAACAACCAGAATGT
ATCATACAACAAGGTAGAATGACAGAAACTGGATACGCACATTCATTAAATGCTTATGATGCGATTCTATTACAGGAAGTTGGGAAGTACTGTT
GGGCATGGGGTAGAGATCAGGAGGAGGCACTGGTTGGATTTCTGGGCTACTCTTTGTGATCTTTTGCAACTTAGGTGGAAGCATAGAGCTATTC
ACTTGATGATAACTTATTGCAAAATACCTAAGATTTATTCTTCTTCAAGTAAGTCTTAAATTGTCTTTGTCCATCAAAGACAAGGTTTATAAGA
GCTAAAAAGATCCTTAGACATTCTGCCGAGCTTACTGGGAACAATTCTAGGGTTAAGGAGACTGAGTCAAATAGAGAGACCACAGTGACAAAAG
AAAAAAAAAAGCAGCATGGAGAAGAAAACTGGAAGTATCTCCTAGCACCAAAGGATGGAAACAAATCCCACATCATGGGAGAGTTAAGCCTTCC
TTCAGACTTTTATAATTGATAATCTCAGTGGTGGGTGGGCCAAAGCATGCAGAGAAGTATAAGCAGAAAGGACTACATCAGAAAAGCTAACATC
AGGCAGTGATCTGGTGAGAGTTCAGATCTGTCCTATTTACCTCTTGAAGGTAATACTGTCTCCTATTATTTGGGATGCTTTCCACAATGTGGAT
GGGAGGGAATACTGTAGGCCATTGGACCCTGCCTAGTTTTCCATCTAGGAAGGAGTCATTCTAGGTTCCCAAAAGAATGAGCCGGACTATAGAT
TGCAAACTCAGCTGTTGACCTCAATGGAAGAGGGTTGGTTTAATGGAGGACAGCAATAAAGAGGGGCTTCTGGAAGGAGTGGGTCTAAGTAGGA
CCACTGTTGATGCACAAGGCATACCTAGGGGAAAATGGTTAGTTTTCTTGGCAT
MOUSE SEQUENCE—mRNA
ATGTCATCTGCACTTGCTTACATGCTGCTTGTCCTCAGCATCAGCCTCCTGAATGGACAGTCACCACCTGGAAAACCTGAAATCCACAAATGTC
GTTCCCCTGACAAGGAAACATTCACCTGCTGGTGGAATCCTGGGTCAGATGGAGGACTCCCCACCAATTATTCATTGACATACAGCAAAGAAGG
AGAGAAAAACACCTATGAATGTCCAGACTACAAAACCAGTGGCCCCAATTCCTGTTTCTTTAGCAAGCAGTACACTTCCATATGGAAAATATAC
ATCATCACAGTAAATGCCACGAACGAAATGGGAAGCAGTACCTCGGATCCACTTTATGTGGATGTGACTTACATTGTTGAACCAGAGCCTCCTC
GGAACCTGACTTTAGAAGTGAAACAACTAAAAGACAAAAAAACATATCTGTGGGTAAAATGGTTGCCACCTACCATAACTGATGTAAAAACTGG
TTGGTTTACAATGGAATATGAAATTCGATTAAAGTCTGAAGAAGCAGATGAGTGGGAGATCCACTTCACAGGTCATCAAACACAATTTAAGGTT
TTTGACTTATATCCAGGACAAAAGTATCTTGTCCAGACTCGCTGCAAGCCAGACCATGGATACTGGAGTAGATGGGGCCAGGAGAAATCTATTG
AAATACCAAATGACTTCACCTTGAAAGACACAACTGTGTGGATCATTGTGGCCGTTCTCTCTGCTGTCATCTGTTTGATTATGGTCTGGGCAGT
GGCTTTGAAGGGTTATAGCATGATGACCTGCATCTTTCCACCAGTTCCGGGGCCAAAAATAAAAGGATTTGATACTCATCTGCTAGAGAAGGGC
AAGTCTGAAGAACTGCTGAGTGCCTTGGGGTGCCAAGACTTTCCCCCCACTTCTGACTGTGAGGACTTGCTGGTGGAGTTCTTGGAAGTGGATG
ACAATGAGGACGAGCGGCTAATGCCATCCCATTCCAAAGAGTATCCGGGTCAAGGTGTTAAACCCACACACCTAGATCCTGACAGTGACTCTGG
TCATGGAAGCTATGACAGCCATTCTCTTTTGTCTGAAAAGTGTGAGGAGCCCCAGGCCTACCCCCCTGCGTTCCACATCCCTGAGATCACTGAG
AAGCCAGAGAATCCTGAGGCAAATATTCCTCCCACCCCAAATCCCCAAAATAACACCCCCAATTGTCATACAGATACATCCAAATCTACAACAT
GGCCTTTACCACCTGGCCAACACACGCGCAGATCTCCTTACCACAGCATTGCCGATGTGTGCAAGCTAGCTGGAAGTCCTGGAGATACACTGGA
CTCTTTCTTGGACAAAGCAGAGGAAAATGTTCTAAAGTTGTCTGAAGATGCTGGAGAGGAAGAAGTGGCTGTGCAAGAAGGGGCCAAAAGCTTC
CCTTCTGACAAACAAAACACATCTTGGCCACCACTCCAGGAGAAAGGCCCCATTGTCTATGCTAAACCCCCAGATTACGTGGAGATTCACAAAG
TCAACAAAGACGGAGTGCTATCATTACTCCCCAAGCAGAGAGAAAACCACCAGACAGAAAACCCTGGGGTTCCTGAAACCAGTAAGGAGTATGC
CAAGGTATCTGGGGTCACGGATAACAACATCCTGGTGTTAGTGCCAGACTCACGAGCCCAGAACACAGCGTTGTTCGAGGAATCAGCCAAGAAG
GTTCCACCATCGCTTGAACAGAACCAATCTGAGAAAGATCTGGCCAGCTTTACTGCAACCTCAAGCAACTGCAGACTCCAACTGGGCAGGCTGG
ATTACCTGGATCCTACGTGCTTCATGCACTCCTTTCACTGAGAGC
MOUSE SEQUENCE—CODING
ATGTCATCTGCACTTGCTTACATGCTGCTTGTCCTCAGCATCAGCCTCCTGAATGGACAGTCACCACCTGGAAAACCTGAAATCCACAAATGTC
GTTCCCCTGACAAGGAAACATTCACCTGCTGGTGGAATCCTGGGTCAGATGGAGGACTCCCCACCAATTATTCATTGACATACAGCAAAGAAGG
AGAGAAAAACACCTATGAATGTCCAGACTACAAAACCAGTGGCCCCAATTCCTGTTTCTTTAGCAAGCAGTACACTTCCATATGGAAAATATAC
ATCATCACAGTAAATGCCACGAACGAAATGGGAAGCAGTACCTCGGATCCACTTTATGTGGATGTGACTTACATTGTTGAACCAGAGCCTCCTC
GGAACCTGACTTTAGAAGTGAAACAACTAAAAGACAAAAAAACATATCTGTGGGTAAAATGGTTGCCACCTACCATAACTGATGTAAAAACTGG
TTGGTTTACAATGGAATATGAAATTCGATTAAAGTCTGAAGAAGCAGATGAGTGGGAGATCCACTTCACAGGTCATCAAACACAATTTAAGGTT
TTTGACTTATATCCAGGACAAAAGTATCTTGTCCAGACTCGCTGCAAGCCAGACCATGGATACTGGAGTAGATGGGGCCAGGAGAAATCTATTG
AAATACCAAATGACTTCACCTTGAAAGACACAACTGTGTGGATCATTGTGGCCGTTCTCTCTGCTGTCATCTGTTTGATTATGGTCTGGGCAGT
GGCTTTGAAGGGTTATAGCATGATGACCTGCATCTTTCCACCAGTTCCGGGGCCAAAAATAAAAGGATTTGATACTCATCTGCTAGAGAAGGGC
AAGTCTGAAGAACTGCTGAGTGCCTTGGGGTGCCAAGACTTTCCCCCCACTTCTGACTGTGAGGACTTGCTGGTGGAGTTCTTGGAAGTGGATG
ACAATGAGGACGAGCGGCTAATGCCATCCCATTCCAAAGAGTATCCGGGTCAAGGTGTTAAACCCACACACCTAGATCCTGACAGTGACTCTGG
TCATGGAAGCTATGACAGCCATTCTCTTTTGTCTGAAAAGTGTGAGGAGCCCCAGGCCTACCCCCCTGCGTTCCACATCCCTGAGATCACTGAG
AAGCCAGAGAATCCTGAGGCAAATATTCCTCCCACCCCAAATCCCCAAAATAACACCCCCAATTGTCATACAGATACATCCAAATCTACAACAT
GGCCTTTACCACCTGGCCAACACACGCGCAGATCTCCTTACCACAGCATTGCCGATGTGTGCAAGCTAGCTGGAAGTCCTGGAGATACACTGGA
CTCTTTCTTGGACAAAGCAGAGGAAAATGTTCTAAAGTTGTCTGAAGATGCTGGAGAGGAAGAAGTGGCTGTGCAAGAAGGGGCCAAAAGCTTC
CCTTCTGACAAACAAAACACATCTTGGCCACCACTCCAGGAGAAAGGCCCCATTGTCTATGCTAAACCCCCAGATTACGTGGAGATTCACAAAG
TCAACAAAGACGGAGTGCTATCATTACTCCCCAAGCAGAGAGAAAACCACCAGACAGAAAACCCTGGGGTTCCTGAAACCAGTAAGGAGTATGC
CAAGGTATCTGGGGTCACGGATAACAACATCCTGGTGTTAGTGCCAGACTCACGAGCCCAGAACACAGCGTTGTTCGAGGAATCAGCCAAGAAG
GTTCCACCATCGCTTGAACAGAACCAATCTGAGAAAGATCTGGCCAGCTTTACTGCAACCTCAAGCAACTGCAGACTCCAACTGGGCAGGCTGG
ATTACCTGGATCCTACGTGCTTCATGCACTCCTTTCACTGA
HUMAN SEQUENCE—GENOMIC
CTTCCCCAGGGACACATATTGAATAGGCCTGCAATGGGGCCTTGGAATTTGCATTTTAATGGCCACCTCATGTTATTCTGATGCACAGCTATGT
TTGAGAATTATGGCTCTAAAGCAAAGCTCTCCAAGAAAGAAGTACATACCTAGGTATACAAGATGATCCAGTGAGTTGAAGTATGAAAATAATA
TAGTAGTTATTTGGGGTATTTTTTCTAAGAAAGAAACTAATCTTTACTGGTATTTAGATTGACAATAGTGCCTTCACTGAGTTCTCAAATTAGC
TCAACACAAGTTCTGGAAACTACACAAGATAATCTCCAGAATCAAAAAGAGTTCAGCAAACACACGGGGGTTGCCATGGTACCTCCACTGCTTG
ATTGCCATTTATGTGCTACAGGTTTTCATCAATGCAGTTTATTTTATAAAAGCAAGACCTTTAGAATATGCAACTTGACAAAATAGGGAGTGAG
TATGAAAATGTCTGGTACCACACAGAAGTTTCTTGTTTAATTCATGACGAAGTAGTAAAAAAGTTGTTGGATTTAAAAATGAGTTGTCCATTTC
CTTTTACAAAACGATAATTGTTCCCAAAGTGTAAATCTCAAATGCATGATGCTGAGTGAAAGAATGCTACATACTGTACAATTCTACTTACATG
GCAGCCTGGAAAAGGGAAACTATAAGGATAGAGAACAGATTAATGGTTGCCAGGGGACAGGGTAGGGACAGGGTTTGGCTACGCAGAAGCATGA
GCGTATTTTGAGGGGCCGGGCTGCGGGGCGGTGGGGTGTGCGGGAGTAATGAAACCATTCTACATCTACATTGTGATGGTTATGTGACTTTTTG
CATTTGTCAAAGCTCCTAGAGATATACACTAATGAGTATGAATTTTATAGCAAGTTACACCCTAAAAACATTCACTTAAAAAAAAGTAAGTTTT
GCTGAACAAAAAGAAAATTTCTACCACCTTTCATGCAGTATATTTTTCAGCCATAATAGGTGTAAAACTAAAAACTGAAAATAAACTAAACTTA
GAACCAGATCTGCATATCACTGTGTCTCAAAGTGTTAAGCCAACATCTTTTTTTTTTAAAAAAAGCATACTTAATCACATTATTCTCATTAAAA
TTTTGATAATTAATAATTTCAGAACTAAATGGTTTATACCATTAAAAATAAAATTATTTATTTATTATTTTAAATAATTTTGACTTTTATTTTA
GATTCGGGAGTACGTGTGCAGGTTTGTTACATGGATGTATTGCATGACACTGAGGTTTGGGGTATGAATGATCACGTCATCCAATGAGCGCAGT
ACCGAATAGGTAGTTTTCAGTCCTTGCCCCGCTCCTTCCCTTCCCTTCTAGAATTCCTCATTGTCTATTGTTCCTATCTTTACGTCCATGCATA
CTCAAAGTTTAGCTCCCACTTATAATAGAGAACATGAGGTATTTGGTTTTCTGTTCCTGCATTAATTTGCTTAGGATAATGGCCTCCAGCTGCA
TCCATCTTACTGCCAAGTACATGATTTGGGGTTTTTATGGCTGTGTAGTATTCCATGGTCAAATTTTTGTTTCATCTTATTTCATTCTTTTAAA
ATTTCTACTTTCATTCATTTATAATACAGTAGTACATATATATAATTTATGACTAAAAATACATATGTATTTGGAAAGGCATGCTAAAAATGAT
TTTACTGCTAAGATGATAAACAGAAAACTTTGGAGGTCACTTCTCTAGAAAATAACTCTTCAAAATTTAATGTACGTAGGAACTACCTAGGGAT
CTTGTTAAAATATAAAGTCTGATGCAGCAGGAAGTCTCTCACCAGATCAAATGCTGGCACCTTGATCTTGGACTTCCCAGTCTCCAGGACTGTG
AGAAATAAATTTATGTTCTTTATAAATTAAAGAAAAAATACAAAGTCTGATTCACAAAGTAGGGATGGGGTCTGAGATCTTGCATTTCTAATAA
GATTATGATTAATCTAATAAGTTGAAATCATTTGTTTTGGTTTAAACTAATTACCAAGTCAATTCATTCTAATACCTTCCGGGTTCACTCATAG
ACTAGGCCTAGGGTTAAAATGATAATTAGTATGATGCCAGTGCACCTGGCCCTAAGACTATACTTTGAATAGCAGGACTCAGACCACAGTAGCC
CGAAGACATCTATGCAGCTATTATAGAGACTAACAACTGCTGCATTTGATTCAGCAGACTCAGTTTTCCCTCTTTGGGTTTGGGAGACATGAAT
TCTTATCAGTCAATAGGCCAGCTCTCTTAGCATCTTCCCATGGAGCAAATGATTCCCATTCCCATGCTCATAAAAGCTGATTTTACTTAGGTCA
CTGCTTATCCACCAGCGTGGGTTTCCTTAGAAGGAAGGACACATTGTCTAATAATAGCAGTGCCTTATATTTACACATGGTTTATAATTAACCA
AATGTCAACTCATATCAGCCTCACAATGACCTTTGGAGGTAGAACAAAGCAGATGTATTATAAACCCATTTGCAAGTGCTACATGACAGAAGCA
AGCACAGTGCTTTCTTTCAGAGTTGGAGTGATCAGGCAAAGCTTTAAGAAGAAAGTACCATGGAGTCTGAAGATCAAGAATGGGTAAGGTTTTT
AGATGAAGGGTGGGAAATAACATTCCAAGAAGAGAGGATGGATTCAAACACACTGTAGATATGTATTGAGCTACTACTACTCTAAAGCCCAATG
AAATCAGCAGGAGTAAAGATGTTCTAACTTTATTCTGTGGGAAGTAGATGCCATTAAAAATTTTTAATTCAGGGACTAATGTAATCAAGTTTAC
CATGAAAGCATTTGAGTTTGATGTAGGCAAGACTGGTGAAGATGAGTCCAGCCAGGGGGGCTATTGAAGAGGAAGAAGTAAAAAATGATGAAGG
CACAAATTGAGGCAATAACAGTGGAAGGGACAAAAATATTTAAGGACTTCATTCCTTTTTTTTTTTTTTATTATACTTTAAGTTTTAGGGTACA
TGTGCACATTGTGCAGGTTAGTTACATATGTATACATGTGCCATGCTGGTGCGCTGCACCCACTAACTCGTCATCTAGCATTAAGTATATCTCC
CGATGCTATCCCTCCCCCCTCCCCCCACCCCACAACAGTCCCCAGAGTGTGATATTCCCCTTCCTGTGTCCATGTGATCTCATTGTTCAGTTCC
CACCTATGAGTGAGAATATGCGGTGTTTGGTTTTTTGTTCTTGCGATAGTTTACTGAGAATGATGATTTCCAATTTCATCCATGTCCCTACAAA
GGACATGAACTCATCATTTTTTATGGCTGCATAGTATTCCATGGTGTATATGTGCCACATTTTCTTAATCCAGAAGGACTTCATTCCTAATTGG
ATGTAGAGTGTAACAGAGAGAACACTGACAGTTGAAGCCATAAAAAAAAAATAATAAGCTTTTATATCTCAAGGCCCATAAACAAGTGCATGCC
CTTTGACCTAGTTGGGCCTCACCTGGTGTTTTAGACAAAGGAAATTAATTCCCAAAGGCAACTTTTTTTTCTACACTTCCCATAAGTATGTTTA
TTACAGCATTATTTATAAGAGTAAAAATTTGGGAACAATCTAAATGTTCATAATATAGAATGATTACATAAATTATTGTGCAAAAAGATTATAA
TGATAAGATTACATTAAAAAATTGAAGGATACTTTTAAAAAGATATAAATAGATATATTTCCTGGATGGGAAGAATTAATGCCCTAACAATATC
AATTTTTCTCAAATTAATATATAAATGTAATGTAATGCCAAAAAAAGATTTTGAATTGAACAAATTGACCCTAAAATTTTATGAAAAAATAAAT
ATAGTGGACTTATCAAGACAATTTCAAAAGGAAAGTGACAGTGTGCTTTGCACTAACAGGTATTGTACTGTTTTTACAGTTACAGTAAACAAAA
GTCTATATTTTTGGCATATAAATAGGCATCTCAAAGAAACAGAGTAGTGTCCCGAATTCATATAAATATATATCAAAATGTAACACATGATATG
TGACACTAGAAATCAGTGGAGAAATTATGAATTTTTCTATAAGGGGTAAAATTAAAAATAAAATTCAGGAGAAAATTAACTGTCTACCTTATGT
AATAGTTAAAGATCTATTAAAAAAAAAACAATTTAACAGAGTTAATTGCAAAGAAGCAATACAAAAAACCCAAAAGCTGTAACAGAAAATAATT
GACAGATTTGAGTACATGAAAATTAAAAACTTTTGTGCAATGAAACAGTCCATAAACAATATCAAAGGAAGCCTGACAGGCACAAATGTTTGCT
ATACATTGAACAAAGTGTTAGTATGTTTAATAATAAAAGAGATGCCAAAAATTAATTTTAAAAAATATGGAGGCCAGGCACGGTGGCTCACGCC
TGTAATCCCAGCACTTTGGGAGGCTGAGGTTGGTGGATCATGAGGTCAGGAGATTGAGACCATCCTGGCTAACACGGTGAAACCCCGTCTCTAC
TAAAAATACAAAAAATTAGCCGGGTGTGGTGGCAGGCGCCTGTAGTCCCAGCTACTCAGGAGGCTGAGGCAGGAGAATAGCGTGAACCTGGGAG
GCAGAGCTTGCAGTGAGCCGAGATTGCACCACTGCACTCCAGCCTGGGCAACAGTGCAAGATTCCATCTGAGAAAAAAAATATATATATATACG
GGCCAGGTGTGGTGGCTCACAACTGTAATCCCAGCACTTTGGGAGGCCGAGGTGGGCGAATCAGGAGGTCAGGAGTTCAAGACCAGCCAGGCCA
ACATGTGAAACCCTGTCTCTATTAAAAACAGAAAGAAATGCTGGTCGTGGTGGTGGGCACCTGTAATCCCAGCTGCTCAGGAGGCTGAGGCAGG
AAAATTGCTTGAACCTGGAAAGTGGAGATTGCAGTGAACCGAGATCATGCCACTGCACTCCAGCCCAGGTGACAGTGCAAGACTCCATCTCAAA
AGAAAAACAGGCATAAGATTGAACAGCCACAACATAAGAGAAAAAGTCAAAATATAATAAGCACATGAACTCAACCTTGAAGGCAATCAGAACA
CTGTGAATTAGAACAACAATGAGCTATCATATTTTGCTTATCAGACTAGCAAGATAAGATTGATAAAGTCCAGTGCTAGCAAAAATGTGAGTAA
ACATCCTTGGTAAGAGTGGACACTGAAACATTTGTGGATAAAAATTGGAGGTATATATGAAAATTTAAAATGTTCCTACCCTGTGGCTCAGTAA
TCTCATTTCTAGGGCTTTCTCCAACAACATATTTGGAGAGCAATTTGGTAGCTATCAAAATTTTAATGTTCATTCTCTTTGAGCCAACCAAGTC
ATTTATTTTTTATTTTATTTATTTATTTATTTATCTATTTATTTTTTTGAGACACAGTCTCAATCTGTTGCCCATGCTGGAGTGCCATGGCACT
ATCTCGGCTCCCTGCAACCTCTGCCTCCCAGGTTCAAGCGATCCTCTGGCCTCAGCCTCCCTAGCAGCTGGGACTACAGGCGTGCACCACCACT
CCTGGCTAATTTTTGTATTTTTAGTAGAGAGAGGGTTTCACCATGTTGGGCAAGCTGGTGTGGAACTCCTGACCTCAGGTGATCTGCCTGCCTT
GGCCTTCCAAAGTGTTGGGATTACAGGCAGACGCGAGCCAACCTGCCTGCCAATCAGGCCATTCTAAGTATCTATCCTAAAGTCCATGCAAAGA
CAATCCTAGCAGCACTTCTTAAAATAAAAAGCTGGAATTACTAAAGACCTAACAATGGAAGAATGGTAGAATATATTACAAACTATGTAACCAT
TAAAAAAAAATGGAGTAAATCTATACATAGTTACCTGGAATATGTGATTTCATTAAGTTTAAAAAAGAAAAGTCACAGAAATTATTGCATTTTG
AATTTTTAAAATATTTGCATGTGCATACAGAGACATGACTATATACACATGTAAAAAAGGAGGCAACCACCAATACGTCAATGGTGGTTACCTC
TGAGACAGGATTAGATGAAGGAAGGAGGATGTGTGATTTGGAAGTACTATATTTTACTGTATAGATAAAGATGTAGATTTCTTTTTAACAGTGC
TAGGATTTGGGGTAGTTTTTTAATCTTTGGATATTTTTAGCAGTTTTCTATTTTTTTTCTAAAGAAAGAAACATAGTTCTCCCACATATGGACA
TGCCATTCTTCTGAGGATGCTTAAGTTGACCCCAGGAGGAGCCCTAGCTGCTGTTCCCCATTAGAGGCCCCTTTTCAGCAGGAAGTAGCCAGAA
AGAGTCCTTGCCCAAAACCCCCTAACAGCAGTTAGTGTGATATCGCCACAGGGAGGAATGTGGCATGAGTTATCAAGAAATTATTTTAGGCAGA
TAGAGAGTAAAAGGGGTCCTTGGGAAGTTTTCATTTTTTAAAGTATCTCCAGAAAAGTTTCTTGTAAAACCCCGGCTTTTAGAGCCAGGCGGGC
AACCTTTGATATGCAAACCTAGGCCATTAGAAACTGGGTTCACCCAAACATGCCGATTCCAGGGACCTTCTTGCCCTTGCCCCACATGTTCCTG
GCAACATGGCCGCCCCCACATATCCCTATTTGTGTAGAACATCATGGCGCCCCGCATTTGCATATTAAAAGGCTAGAGTGGGAGGGCCAGCTCT
TTCATGGGCTACGTGAATTACGTGCCTAGTCAAACCAATCCCCTGAGCCCTAGGCAAATCAAACACCGCCTCCTCCAGCCTCTGCTTATATACC
TGGCTGGTATCCGCAGCAAGTGGGGACTTACTCTTTTGGCTTTGGAGCGCCCCCCTCCCTCTGTCTCTGTACAGGGAAGCTTCTTCTTTCTTCC
TTCTCCCTTTCCTTCTTGACCCTTCTTGCCTATTAACCTCTCCACTCCTTAAAACCAAAAAAAAAAAAAAAAAAAAGAAATGTGGTTCTCAAAG
AAGACAAATGAAGTAGCAATGATAAAACTCCTTCCCTATTTGAGAAGGAGGAGAGAGAAAAGGAGCCACGGCAGAAACCAACAGAGGAATGCCA
CAGACAGAGGGGAGGCTCCCGCCAGAGGATGGCAGAGGCCTGGAAGCCAAGGCGTGAGAACTTCAAGAGGAGGAAGTGAACCGAAGCCCAGTGC
TGCAGAGAGGCCTGGAGGGCCCTCCCAGAGAGCAGCCAGTGAGTGGTAGTGATGAAAGCCAGGTGCAAAGAATTCAAGGGAGCCATTTTCCACC
CTGCCTGTGCATTTTTGTAAAAACAGAGAAGTCAGAGCCCCATTCTAAACCTCCTGCATTAGAAGCTCTGCAAGTGACACCCAGACACAGTACA
TTTTAAACACTCAGGTGATTCTTGTTCACAGCCAAGGTTGAGAACCAGTGAGTTAAGGCACTGAGAGGGAAGTAAGTAAGGAAAGCTGGTGTGC
GTAAGTCATTCTTTCACTAAGTTTGGCAGGTAAAGAAATGAGTGAATTAAACTGTAAAGCCATCAAAAGGGTCAAAGAAGATGTAGAAGTTGTA
GGTTGAGATAAAGAAGCCTATGGGGCAGAAAAGATTAAATATGCAAGAAAGGTAGAGGAAAGGGGAAGGAAAGAAAGGAACTTGATGTTTCTAG
ACTTTGTTAGATGGCACACATTTTACATATGCCATCACCATTATTATTCATTTGACTGGATTTTTGAGGCACATGCTACTGTGTCCAATTTACA
GTGAGAAACCAAGAATAGCAAGGACCTAACCCAGTGCCAGTGCTGGGCTCTTCCTCTGCATCAGGCTGAGAAGCAAGGTCTTGGAGGAAGTAAA
CGGATATGGAGTCCAGGACTCAGGTAGGGAGTCAGCTTGCAAAGGACGAACACGTCCGCTAAGACAGAGATGCAGAAATGAACTAGAGGAGGAC
ATCAGTAAAGAAGCTGTGAAGCTGGAGCACATCAGTGCATAGAGAGAAGGAGATAGTCAAAGAACCACTGTGGGGCAGAAAAGGAAAATATAGT
GGCTCACTGCGGATTTCATCATCTCAGGAAAGCTGGGGCAAGGTAACTCACTGACAATGAGTACCCGTGGCTTTGTGACAGTGCCTGTGTGCTA
CACAACAGAGCAGAAAGCCAGCCCTGCCCTTCTTACCAGTGAGGTATGGATTAGCCACGCAACGACCAGTCTGTAAGCCAGGAGTACAGACGAG
CCAAGCTGCCTGCAGCTGAGTGGGAACCACAAGGAGGAAAAGCCAAGTCCCTGCCTTCTAAGAGCTGACAGTCTGGAGTTACCCAGAGTGATCT
TCTATTTCACTAACTGTTAAAATAGTCTGATTAGAACAACTATTGTAGAATTGAGTGACCCTTTGCTTTGCAAAGGTCATATAGAAATATTCTG
CCACTGAGGATCTCATGTCGCCCAGTGGTTCTGAAAATAAATAGAAGTTGCGTACCTTCATGGACACCCATGAATGAAAGTAAATATACCCTAA
TCATCACAGGAAGCATGTGCGGTCTGCACGTGAAAGATGCATCTCCCATCAGCTAACAGAACCAGGTGGAGTCTATGTTTTAATAAGGACGTGT
TATTGATCGGGAAGTATTAGGTTATTATGCCTAGGTAGTTGCTTCTGGGATTCCAGTCCAAAAGGATCACCAAAGCATCATGGGATTTTCAATG
GCTTCCTGTGGCTTGGCAAATGCTCACTGCTAACTGTGATTTCCCAGTTATCCAGAGTAGCAGTCAAATCAAAGGGGAAAGCCTCATGTTTCAA
TAACTTAAAACAAAACAAAAAAAAAAACGGCCAGGCGCAGTGGCTCACGCCTGTAATCCCAGCACTTCAGGAGGCCAAGGTGGGTGGATCATGA
GGTCAGGAAATCAAGACCATCCTGGCTAACAGGGTGAAACCTCATCTCTACTAAAAATACAAAAAACTAGCCGGACATGGTGGCACGCGCCTGT
AGTCCCACCTACTCGGGAGGCTGAAGCAGGACAATTGCTTGAATCCAGGAGGCAGAGGTTCCAGTGAGCCGAGATGGGGTCACTGCAATCCAGC
CTGAGCGACAGAGCGAGACTCTGTCTCAAAAAAACAAACAAAAACAAACAAAAAAAAAAACTCAGTAACCTTGGTTTAAAGTATTTCCTTGGTG
GCCCTCTGGGTTGTGTAACAATCGGATGCATTAATTATTATGGCATCAAATGACACCAGCAACATACATAGTCAGAAATAACTATTTGATTCAG
GCAGGTGCTATTATTTGTTTCAGCACTGTTAAATGTGGGGTTTTTGTTTTTGCAAGAGAAAAGGCGGTTGTGTGTGGTTTCAAGAAGAAGAAAA
GTCAGAACAACATGCAGCCCATTAGAAAAGATAAATTCTTTATTTGTGGAAAACAACAGAAGTGGGGCTATTTAGGCACCGCCAATGTTTATTA
TATTCATATGGCAGAGAATAAACACAGCACACTAGAAGTTTGCGCTTTGGGTATTAAAAAAAAAAAAAAGCAAAACCTTCTTAGGTCACTGACT
ATCTTCTTTTGCTCCTTTCTATCTGACTCCTCAGGCAAGGTGCAATTCAAATCCCTGGAGAACCTCTCAAATATAGCTGCTGTTCAATGTTACT
CAGAAATAAGGAAAGAAATTTGACACGGAGACACTCCACATCTTGGCTGCCTGGAAAAATATCATCTTTGAGGTCATTCAACTCACTAAGTCAT
TTTTATTATTGTTTTGTCTGTCTATTTTCTGGTACAGTCTCAATGAAAGATATGTGGCCCTAATCATGCAAAACCGATCTGGGGGGAATCAGTT
GTTTCTTCAGGTCTTCGCAGGATTCCAGCTCCCCCAACTACCTCCGTCGCCTTCTAAAACTGGCAGGCTCTGGACGTTTTGCATGCTGAAGAAA
ATCACTGTTTTGCCTCCAGCAAGGAACATAAATGTTGCAACACTGACTCCTCCTCTCATGAAGAAAGAGTGAACAAGTGCACCGAGTTGAGCTT
CTTCTCACAGAGCCACCGGGCTGGGGACCGGGGAGCGGAGGCTGAAATCCCCAGACGCCGGTTTTCTGGGCTGGGCTTTCTGCCTTACTCACTC
CTTCTCCCTCTTTCTGGATTTTACCGACCGTTCGCGAAACAGCTTTCCACACAATGGAGCTTCATGTCCTCGTGCAGGAAGTACTCATCGACTG
ATGTGGCAGACTTTGCTCCCTGGCAAAAGTGAGTACCCATGGCGAGCTGAGGCCGCCTCCGACGGCGCGCGGCAGGGGCTGCGCGGGCGGCCGC
TCTCCGGGATGCGGGTTCGAGGCCCGCGTGCTGAAGGGGAGAGCGGGGCCACCGGCGCGGGCTCCTGGAGAGAACGCCAGCCAGGAGCCAGAGT
CGGCAGCCGGCGGGGAAGCCCGGGATCCGCCCGCCTCCCGCCCGGATGTCCCTTCTCAGAGGGGCTAAAAAATACTCTTAAGGTGCTTTGCCTC
CACCACTGCCCCCAACCCCAGCTTTTACAAACTGCACTCGGCTCGCATTCGCCTTTGTCAAAGGCATCTTCACCTGTATCCTTGAGCATGAGTT
TCGGCGTTGATTGTGGGATTCTCAGAATGCAAATTATTTGCGTCAAAATAAAAAGAAAAGAAAAAAAGAGCGGGGAGGGGGAAGGGGCGCAGAC
AGGTGATGTGATGAAAGCCGGACTCGAGACTCTCCCATCAGCAGCAGCCGCCGCCGCAGCAGCCTCGCGGGCATCCGGCGGGGAAAGTTTAGGG
TTAGCAATGCTGACCTGAAAGGTGTTGCTCCTCCCAAAAGTCAGCGGGGGTAGGGGCAGATGAGCAGCCTCCGGGCCAGAAGCCGGAGCTGGAC
GGAGGCACCTAGGCAGAGGAATTGCAGCCCGGGAGGAAGCTGGAAGGAGAGCCCGCTGGAGAAGGGAGGAGGGAAGGGAAGAGGAGTTGGTTAA
AGTCAGGCAAACTCAGGTAACTGGAGGGCCGGCCCGAGTGCTACGGCAGGGGAAGGCAAGGGCTGGAGTTCTGGCTCTTTGAGTAGAAAACTAT
CCTTTTAGTGACCTCTACCTGTATCCTGGGGAAGCAACCCACACTGCCCTGAAAACAAGCAACGTAGGGGATTCTATGACTGGACTTGAAAATG
GTTGCAAAACATTATTATTAGTCTGGAGGGATCACTTTGTCTTCATTTGTCAAGTTGACCGGGCAAGATTTCCCACCTGAAATCGTGCTTCACT
TGACCAGGGAAGTGCTGCTAATCCCAATCCTGACCATCAGCTGTGCAAACTTGGGCAAGAAACTTACCCTCTCTGGGCTTCGATTTTTTTTCTG
CAAAGTTTAGGGAATTAATTAATTATATATATATATTATAGAAATATATATAATATATATATAAATGTTTAATGAGGTGGAAGTGCTGAATAGT
GCAAGAGAGGAGCAGATGAAAATATTAATTTCAGTGGTTCTTAAAAATTAGGGGTGATAGGTCCTTTTGAGAATTAGCTGTGAGTCCTCTTCCT
GGGGGAAAATGCATATGGCATGTACATAAAATTTTACATGCAATCCCTGAGGGTTTGTGGATACTCCCTGAAAGCCATGCCTGAATCCCAGGTG
ACAATCCCTTATTCCTCCTCCTGTATCAAGACCACCCAGCCAACTCCTTGCTGCTGGTTGGGCTGACTGTGGGAGTAATCCACAGCCTTCTTGG
GGGTCTGGAGTCAGCCACGCTGCTGAAGGAGACAGCTCCCCTGTCATGGGTGAGCTCCCCACATGCTCTCTCCAGAGCTCTACCAGCCTCTGAC
TCCAGGGGACCCCTCCTCACAACCCTTTTTAATGACTCTCCTAAGGCGACAGTCCTTACCCCTAGAATTTTCAGTCCCACCAACCCTGGAGAGC
CATCTAAAGAGGATCGCTGTCTGAATCAGAGTGCTCTTCAGGTCACCTAGCTGGATGGCAGGGAGGCCAAGGCTTTGTTTGGAAATGAAGCAGT
CTTTGCTCCCTGAGGCATAGTATGAGTAGGCAGCCATATTCTTTTGAACCTCTGTACTTTGCCTTTAGAGGACTAAGGACTCACATCTCTGGGG
ACCCTGGGTGTTCCACTAGCAGTGCCAGCAAGGCACATCAGAAAACACACAGGGCTTTGGAGCTCAACAGACTGGGCTTCAGATCTTGACTCCC
ACACTGAGCAAGCCTCTGAGTTTTCCTGAGCTTCACTTTTCCTGTCAGCACAATGAAAATAATGCTGCTTTTTTTTTTTTTTTTTTGCATGGTT
GTTGGGAGAATGGAAATACTATGAATCATGCCTAGCCCATAGTCAATGATAAACTTTTTCTTAATATTAATGTGGCACCATAAATAAGTGTTCA
ATAGTCTCATGCTTTTAAGAACAGCTTACTCTCTCTCTCCTATGGGGCTTACATTAGTAGAAGAATGAGTAAAATTAGTTCTGGTCTAGTTATG
CACAAAAAGGAGTGGTGGCCTCAGAATACAGAGTCAGACTGGCTGTTCGAATTGTGCAGTGTCAAGGAGAGCACTATGTATTGCCGGCAAGTAA
AGTATGTCCAGACCGGCGTGGATACTTGACCTGTCTCTCAGTTCTCTTTTGACCCATAATCTTGAGTACAAAACAGCTAGTGCTGTGCCTGCAG
CTAAAAACAGCCAGGCATGGAAAGAAGTGTTGTGGCTGGGTCCATCTTCAGAAACTGTAGAGAGAAATATCTCCATTGTCATCTTTCACAGTTT
GGCACAGATGTAGCCAACTCAGCATGTACTCTCTCTCCATGGGGTCCTGGTGCCTCCATTGCAAGTGGTCAGATGATGTAGCAATGAAAGAAGG
AAACATCGGAAACTTAAAAGGAAATTGGATATCCACAGAGTAGCCAAAGATCTATCAGTATACTTTGGTGAGGTATTCCTGCATGCAAAGATAG
CAATATTCATGCCTACTATTTACTGAGCCTTTAAGATGTGCTAAGCACTTTCACACATTGCCTCAATTAATTCTCATAGCAAGATTGCAGAGTA
TGAGTTATCATTTAAAGCTTTACCAGTAAAGAAACTGAAACATGGAAATGTACATACACCCCCCAACCCCACACCCCACATCCGCCTAGCTCAG
GAAAGGCAGAACCAGGATCCAAGCCCAGGTCTGTCTGACTTTGGCACCCACTCTTTTTCCACTACCCAGTGCTCCCAAGCTCCCCAATATCAGG
CACTTGCATCAGCCAACCGGATTAAGATTCTTCTAAAAGGCAGGAATAGCCAAATCTAGTGGGTGCTTTTAAGTGGTGTGAACATGTGCTTGGC
TTGTGATACATATTAACGGGTAGCAACAACAAGATCACCTATGGGAGGATAATATTCCTGACCCTTCCCAAGCCCAGCCATTAGACTCAAGATT
GGCAATATCCAACATTAGCAGGTGAGGCTGGCGCTCTGTCACTGACTTCCTTGGTAATCCTTTTAGTTAAACAGAGCTCTGGAATGTTCATTCT
CTACCACCTTTAGAATCAGAACACCACTTACCTTTACCTCCCTGTGCTGAGCACGTTTTCATTCATTTATTCGACAAGCATTTGCTGAGCTCTG
TTTTGTGCTGGGCACTTGATGACACAGTGGGGAATGAGACACACAGGATCCCAGCCCTTGTGGAGCTTCTATGCCAGAGAGAGTGACAGAAAAC
AGTCGGGTGAACAAATACATAAATAAAATCATTTCAGATACTGATAAATGCTATGAAGAAAATAGAGGCTGAAGGCTACAGAGGGACAGGGCGT
GGGAGCAACTTTCAACAGGGAAGTCAGAGAAGACTGCCATACAAAGGATACCCTTGAACTTAAACCCGCATTTTCAAAAAGGGAGCCAGCCCTG
TGAAAGTCTAGGGAAAGGGCTACCTTAGCAGAACTCTCAAGAGAAAGTTCTTAAATTGGAGTAAATGAAGCAGTAAAGCTTAAAGTCTGAAGAT
GGGCCCTTTAGAAGTTGACTTGTATATAGGGGGAATTATTATTTGAATGCCTACTATACTATTCTAAGCTGTGCTGTATACTCTGTTTATCTTC
CCAATGATCTGGTGAGTTAGCTATTTTTGTTTTTATTTTACAGTTGAAGAAACCAAGAGTCATCAAGGTTAAGGAGCTAGTCCAAGGTTATGGA
GCTAGTCCAAGGTTATGAAGCTGGTATTTGAATCCCTTAGTCTATTTCACTCCAAAGCCCACAACTACTTGCTAACACAGCATGTGGGCTTTGA
GATAATGTCTTTTGGAGAAAGTAAGGAAGCCGATAGAAGGAGAATATCAAAAGTAAATTAAGTAGCTGAATGCTGACAACGATTTGTATTTGAT
GTCCCAAATTCCAGTTAGTGCACCTACAACTGAACTGTCCAATACAGTAGCCACTAGCCACATGTGCCTATTGAGCACTTGAAATGTGGCTATT
TCATATTGAGATGTACTGTAAATGTACAACGCACCCCAGATTTTTAAGACCTAGTATTCCCCCAAAAAAGTAAACTGTCTTGGCCAGGTGTGGT
GGCTCATGCCTGTAATCCAAGCACTTTAGGAGGCCAAGGCGGGTGGATTGCCTGAGCTCAGGAATTCGAGACCAGCCTGGGGAACACGGTGAAA
CCCCGTCTCTATTAAAAATGCAAAAATTAGGCAGGCATGGTGGTGGGTGCCTGCAATCCCAGCTACTTGGGAGGCTGAGGCAGGAAAGTCTCTT
GAACCCGGGAGGCGGAGGTTGCAGTGAGCCAAGATTGCGCCATTGCACTCCAGCCTGGGTGAAAGAGTGAGACTCCGTCTCAAACAAAAACAAA
AACAAAAGTAAACTGTCTCAATTTTTATATTGATTGTGTGTTGAAATGATAATATTTTAGGTATACTAGTTTAAATAAAATATATTAAAATTAA
TTTAACCTTTTTCCTTTAATGTGGCTTAATAGAAATGTTTAAATTACATATGTGACTCACATGATATTTCCGTTGAACAGCACTGATCTAGACT
AATAAGTTGACAAATACATGATAGGACCTTTTTCCCCCTTCTTCCAAACGTGACTTCTTCCTCTGTTCTTGTGTCAGGCTCCTTCTCATCTGCC
CCCATATTCAGTTATTTGGTGAAGCACGAGAGAAAAGAGGTCCCTCTTCCTTCTCCCCCACCCTCATGCTGCTGGTTAGCAGGTGATCATTTGA
AGTCCACTATACCATGTCACTTTGGGATCTGATAAAATAGACTCAGTGATTATATTCATCACCAAGACCAATCCTCTCACTCAGGTAAATCTCA
AGAGGCTTTCAAACGTCCTACAATGAAAGGGTCATTTTAAATCTGGATTTAAAGTTTTACCTTTAAATCACTTCCTAGTACATTGTCTTTGTAG
TTTTGGGCACTTTGTAAAATCAGACGAGGAGACAACTAGAGCTTCATGTTTTTGTGCAAAGCTCATTAAATAAGTAATCTCATGCAGTAATAAA
TGTTTCTCCCTGTCCTCTTGCTTAAAACAGCATGAACAAAAAGTAGTCCCAGTTGCCAGAAGGGCTACTCCAGGGGCATCACAAAAGACCACCT
TTGGTTTCTTTCTTTTGTCCACTAGCTAAAGTGGCCCTCAAATATGTCAATGAAATAAAGATATATTTTTTTTCCTGCAGTTTCATTTATTTCA
AAAAAAAGAGCAGTCAGATTCTGGTATTGCTGGAGAAAGCCTCTGAAGAGCAAAGACCTGGATTCTGCTTTTGTGTACTCTACTGGTTTGCATG
TTACCCTCTCTCTCCCTTTTTTCCCCCAACTTTCTCCCTTACTTCCTGTAGTAAGTTTGGTGTTGATGAGATAATGCCACAAAAAATGCTAATG
GTGAAATGTGCTGTACAAATTGGCCATGGTTATGATTGATAACCTGAATACAAATGGCATCTCTAGACATGTGCCTGCAGGGAGGAGTTGCCTT
TATGTAGCTGGAGCAAAACATTATGTAAGATTCTCTTGGAATAACATTTGTTCATTTCCACTTGAAATCCAAATCTGGGGGAAATGGCAGTAAA
GAAGCTGACAAGAATGAATCTTAGTGTATCTGTCACTGGTAGACTGAGTTCTTTCTCTAGTCTGTATAGGTTCCAGAGAGAGAACACCCAATTG
AAAAAGGACAAGCTGGTTTTATTCACTGGCCAGAAATGGAAAATGAGAGACCTCTTGCTCTAAAAAGTCACCTTCTCCCTAGCTGTGGGAAGCT
GCAGGATTGTGATGAGTTAGATGTGGGGCGGGGAGCTATGTAAACACATGCAGGGAGAAAGTACAGAGGCACAGACACAGATAAACATGTTTCT
CCATGCGTCCTATGGAGAGAAAATGGCGGCAGTTTTCTTCTAGGGATGGGGAGCCCAGCATTGTAACGCTATGTTAATGACCTAAACGGAACAA
GGGGTAGCCAGTTCTGGTTTGCACTGGTTTCCTGCAGGCCTCTTGTATTTGGCAAAGGATCACCAAGCTCCTGCGACATCCCAGGCCATCTGGA
GTTCTTTAAGCAAGAGTATCTATAGATAAAGAAACTAGGGAAGATAACAATTTAAGAAAATAATGAGCTTTCCCAGCTATTTCTCTAGGGCTGC
CCTGGTAATATTTCTACATCAACAGCAACACATATTTTTCAGCATTGTGTAAGCCAGGAAATCTATGCTAGACTCCACGGGGAAAATAGAAGAA
TAAAACATGGTTCTTGCTCTGGAGGAGCATAGTTCCTAGTTGGGAAGGTGGACAGAAACATAAAAGACCAGTAGCATGTAAAGAACATTTTCTA
AGTGTTACATAAATGATGCAAACAAAGTTTTGCAATTCTGGAGGGCAGCATGCCCATAACCTCTAAGGTATATTAAGAAAGACAAAGTAGAAAA
TGGGCATTGAGGGCTGGAAGCCTAAAGACACAAGGAGAGAAGGAGAGAGCCACCTCACATAAGAAAAAATGCACAGTGGTGTGGCAGTGGAAAT
GTACATGGGTTCGGGGGTGCGTGATGTGGAAAAGAACAGTTGGCTTAATCCAGTGGCTCCAGATCTGAGCTTTACATCAGAATCACCTGAGAAG
CTTTTAAAAATTACAAATGCCTCAGCCCCACTCCAGAGCTTCCATTTAATTAGTTTAAGACTGGGGACTGGTCTCAGTGGTGTTTCTGATTGAT
TTTGTTTTGTTTTTGTTTCTGTCTCAATTTCCCAGAATATTCTAATGTGAAGTCAGAGTTGAGAATCACTGAGCAAAAACCAAGATTTTTATAT
CATAGTCATGGGACATAAAATGAAAGAAGTAATCTTTTAAAGGAGATTAAATGGACAACCAAGAGTGTGACCTTTAATTTGTAGAAACTGTATA
TACATGCACATTTGTATGTAGTCTTTTAAATTCACAGATAATTCCTCGTGGTGATTCTGTCTACTTGGATGATGTGGCTGAAAGCCTTTGTAAC
CAGCTGCCTACTCTTGTCCTCTGTGTACTTTGGGGGTGTCCTATAGTGGATCCTAGCTAAGTGGGGCACAATGGGCATCTCACAGGAGCTTGTT
TCTACCAGGCTTTTTGTAATCATTGCCAAGGGCTTTATTATCATGCCTAATAGTGCCCACAGTAGCTATTTTATAAGCATCTGCAATCTTCCCT
CACCTCTCCAACTTTACAAATTGAGAAATGGAGTTTAATTACTTGCCTTGGGCCAGGAGACTAGCTGGTGATGAAGGTGAAATGAGAATTCTCA
GGGAGGTGAAATTAGAATTTCCAGCTACTGAATTCCACATACCTTATTAAACTCCCAAAGTGAGTCTACCCACTCTCCTGTTGGTGTTAGAATG
TTATTTCTTACATGAATGAATCAGTGTCACAGATCAGTAGGTGAAGATGACTGGTTTCCCTAAGCTGCCAACACTGTGATTATCTGAGTGTCAA
TGACCCATAGGCAGTCACAGTGGTCCCGAGAGCCCCGTCCCTGATGCCTGGGAGCTCTCAGTCTAGTGAAGTTTGGCCTTGCATATTTATAGTT
TATCCACTCTAACATCTGGGAAGTGAAGACTCTGGTTGCTATTCCACTGCTCCTAGCCCCACACTTTACGGCTGTTCTGATTTCCCTTCTCTGA
AGCTCTTGTTCACTGAAAGGATATCAGCTTTTTGCTACACCCCTGAAAAAAAGAGGAGAAAGCCCAGCATTTTAAACAGCTGCTTTTTTTTTTC
TTTTGGATGGAGTCTTGCTCTGTCTCCAGGCTGGAGTGCAGTGGCGCGATCTCGGCTCACCGCAACCTCTGCCTCCCGGGTTCAAGCTAGTCTC
CTGCCTCAGCCTCCCGAGTAGCTGGGACTACAGGCACGCGCCACCAAGCCCGGCTAATTTTTGTATTTTTAGTAGAGATGGGGTTTCACCATGT
TGGCCAGGATGGTCTCGATCTCTTGACCTCCTGATCTGCCCGCCTTGGCCTCCCAAAGTGCTGGGATTACAGGCATGAGCCATTGCACCCGGCC
AACAGCTACATTTTAAAGAATCCTTTATATGTGTTGGGAGCTTCTGGGTTCACTGCCCACGGTGAGACTAGGGGTGGGGACGGAGGTTCTTCTG
AAGGACAGTGCGGGGAAAAAGCCAGAGTCTTGGCAATACAGCCTCTTTAGACAAAAAGATAGGTCAATTTGTACCATTCCCAAATGGTTGGGTT
TTCTTTTCTCTGTCTCAAAATCCATTACTGATTTAAAACAATTTCCAAAAGCTTTAGCAATAGCAAAAAGCAAGCCTCTTAAACAGTGTTGCTC
CACACTGATTGCACGTGAGAATCACCTAGGGAGCTTTAGAAACAACTATAGACTCACTGAATCAATATTCTGGGAAGGGGCCTGGACATTCGTA
TTTATTTACAGTTCTCCAGGTTTATAATGTGCATCCAGGGCTAAGAGCTACTACTGGTCAGCTATGATCCAAATCTTAATTACTTATTTGTATT
TCTTCTTTTGCCTTGTTTTAGAGTCTCATGGCCCAGAAAACCATCAAACTATATTTTTGTTGAGTAGCAATTTCATCATTCTGTTCTTTAAAAA
ACATGATTTTGAGCATAATTTAAGTTTGCTACTTCTATAGAGAGACCTCACACTCAGTAGCCTCATCACTAAAATTTTCTGTGTGCAATTTAGA
AATCACATTGCCAGAAGCTTGCTAATTAATTATGAGACTGAAAGATAAAGGTTAAGCTTTCCTGCCACCTATATATTAGATTTCCCAATTGCTA
TGTTAATCTTTAGTTAACCAAGACTTGCTTTCTTGTCTCAGCCATTTTAAAGCCAATTAACTCTGTTCCTGTTCACACCCTTATTAAAAATATA
ATTACAGCAGTAGTCTCCAGGGACTGTCATTGGGTAGGCTTTACCAAGGTAAATTAGCCAAGAAATAACTAGGCTGTGTAACACCCTCTCAGAC
CCTGGAAATCATTTTGGTTGCAGAAGCTACTTAGTATCAGAGTAGGCTGGGGTACTCATAAATTTGGATTTGGAAAAAGCCAATTCCAAGTCAT
CCTAAATGTGGAAGAATCTGTGATTTTGAGAATGATTCTGGAATCAGTTATCCAGGGTCCAGCCAAGTCTGATACACCGATCCTCAGAAGGTTG
TATTAATAACAAAAAGAAACCAGGTTTTTGTATTAGATTTGATGGAAGAGGCTGGGGGACAGAAGGTGATGTAGCATTTTGTTTTCTGGGGTGC
TGAGATTGTGGGATTAGGAGAAAATAAGAAAAAAGATTGCAGGGGAGAACGATGGACAGAGGCCTCCTGTTAGTCAACTATCATGGGCATCAGA
ATTTCTTTATTCACTCAACTAATGTTTATGGAGGTTCTGCTTTGTTCCAAGGACCCATTATAGGAGCTGGAGATAGAAGGATGAAGTTCCTACT
CTAGTGTAGTTTAGATTCCAGTAAGAGGAAACAGATATCCACACTAAAAACAAACAATAAATAAAACCCAATAAATTAGTCAGTACTATGAAAA
AAATGCAATGAACTGAAGGGAATAGAGTTTGCTGAAGGCTGCTTTCAATAGCTTGTACAAGTTGCACTGTCTGAGGAGGTGCCCTTCAAGCTAG
CACATCAATACTAAAAAGAAAAGCATGCCGAGAGCTGGCAGAAAGAAGGCTTCAGGCAGAAAGAACAACAGCAAAAGTCCTAAGACTGGAAAAG
GTCTGAGTTGTTCAGGAAACAGAAAGGAGACCATTGTGGCTGGACCCGAGGTAAGAAGCAATGAGGGAAAGGTGGGAAAAGAGCAAGGAGGGCC
CGAGGTCATGGGATGGAGTTAGGCTGTTAAGTGCTTTGAGAGCCTAACATCCTAACTCATTGGGAGGTTTTCAGCAGGGTAGGAACATGGTTGG
CATTGCCTTTGAGAGCTCTTCCCAGCTTCTGTGTGGAGAATGGCTTGAAGACTGAAAGCAGAGGGTTTGTAGGAGGCTGATCGGAAGTACAAGC
AAGAGGGGAGGGTGGATCATGCCAAGGTGACAGCAAGCAAGGTGGAAGAGGTGTTGGATTCAGGATGGAATTTGGAGGTATGGCCAGCATGGCT
TTCAGATGGAGGGGACCAGCGAAAGTGCTGGAAAACCTAGTCCTGCTCAAGATGATTGTTCCTGCCCTACCCTTGTTTTAGACCCCTTGTTTTG
AACTAAAACAGAATAATATCTAGTGCTATTTAAAAACATTAGGAATTTTAACCTTAACTTGTTTAAGCAGAACAAAACAAATCCCTGCCTGGGG
AGAAACTTCTGTTTGCTGCTTTTGCCTCTTAACCATAAAAATTTCATCCTGTCTCGTAAGACTATTCTGATGGCTTTTGTTGTTTTTGATTACT
GTGCTTTTCGTAAGTGCTGGAAAGGTCACATTCGCATTACTAGTTTATCTCTTTTCTGAGTGTTTTGACACTTTGGCTGTGTTTACCAAGGCCT
TCCTGCTGTCAGACATTTACATGGGGCTTTCTACAGTTGATCAGTTTCTCCATATAAGTGCTAATATAATCGCAAGCATTTAGTGGGCACTGAC
TGTATGCAAGGCCTTGTTTTAAGAATTTCATGTTTTAACTCATTTAGTCCTTCCAGCCACCCTTTATAATAAAAAGTGTGTACTCTTATTATCA
TTTTATAGATGAGGAAACTGAGGCAAAAAAAGAGGTTAAGTCATATGCCCAAGATCATACAGCTGGTAAGTACTAAAGCCAGGTTTTGAACTGA
ATCCATCTGTACAGCTCTGGGTTCCTAGTAACTATGTTGTGCCACTTTTCTATGTAATTGAAGCTGCCACCTTCAGAAGTAGACTTGTTTCTCC
TGCAAAAACCCCAAGGTACCACTAGGAGACACTAAAACTGGGCCGGGCTTCCGGGAGTTGGACTGGTGGGGAGATGGCATAATCTAGAGGCTGA
ATGATCCAACCCCCCTCCAGGAAGGCCTCACATCTAAACCACCTGCCATCTGCCAAGGTGGGAGGATAAACCACCCCCCACCCCGCCGCCAACA
GGGCACTGGCTGGTGAAAGTTGCCAATACCAGAGGAGTCTTGATGAAATGCCCTCTCTTTTCCTCAAACTCTTTCTGCCTACTATTCATAGTAT
CTGTGTTGTACCCACCCACCTGCCACTTATCAAAGACTGAAGAAACCAAAGTTATTCAACAAGCCATGAAGCAAATTCTAATTGCTCCCCACGG
CCATCTGAGGCTGGCCTTTGGAAACTTAAAAAAAAAAAATTAAAAAAAGAGAGTCATATTAATTTATTTGTTACCACATTATTTGCTTTAAAAT
TCTGGTGGCTTCTTTCCAGTGTTTTATTAGTAGAAGCGAAATAAGCCTAGGAAACACAATATTTCATTCTGTCTTCTCTGCTTTTCACTGCATA
TCTACCTTCAAAAATGTCTTCTCTCAAGTTACTTGGTTTCTCAATAACTAGCGTGAAAGTAACAGACCTTCCTTCCTTCCTAATGATAGTAACA
ACAAGGGATTTACAGAATTGAAGTTTATATAAGGAAATGCATTAAGATTTTAACTTAAGTAAGTGAACTATTCAGCAATGGCTATAAAGACAGT
AATTGTGATGATTTGTCTTCTATATTATATGTTTAAACTAGTTATATATTTTAATTATATAAGATGCAATTTTTTTGCTCCAGTTTCCATAGGG
TAATTTTCAACAGAGATTAACATGCGTTCATTGGAAGTGTTGAATTTACCTAATCAAATCATCAATTTGTTTATTCAACTTTAATTTTCAAAAA
AATGTTATTTTAAAATATTATATGATATGTGTCAGGTATTTCTTACGGTTGACTTCCAAATTTAAAACTGCAAAGAAAACAGAAATCCATACAT
ATAGTTGCAGTAAACTATACTCAATTTTGGAAGATTGCAGTGGCAACATCAGTGAATTAAAGTTTGAAAGCTATTTCAAATCTTTGCATATTCT
CCAAATAACTCAGAATCCTACAAAAAGCTAAATATTTAAAAAAATTTATCTTCAAACCAATAATGTAAGTTTCTTCTTTATTGGGATGATTTTG
GTTAAATATTTAGCAGAAAGTAAGGGTGAATATATATTTGATATAGGTATGTCCTAAGCTAAAATCAAGTCATTTAAAGATCAGAGCAATTAAT
GGCAGTTATGTGATCACAGGAATCAGTCTATCTCCTCCCTCAGGGTTTGATTCAATGAGTTTTATTTTTCTCTTAGCAACTTTTCTTATTTTAC
TTTTCAAAGGACATCATTGAAACCATAATAGTGTTCATATCTGTGACTTTTAAATTTTTATTTATTTTTATGCATGGTTTAAAATTCATTTAGT
TCATTTAAAATTCAAAGATATAAAAGAGCATTCATAAAAATGTCCCCCTCTCCTGTCCTCCAGCCTTTACAAAAATATACCATATATATTAATA
TGGGCTAAATACCCTTTTAAAGTTATCTCACACGCAAGATGCTATGACTGTGTGAAGCCGGAGCCTACAGACTATTGCTGATCCCCCAGTAGTA
GTAGCAGTGACTCTTTAGTGGCCCTGGAGCTTCCTTTCTAAACTCAAGTGTATATTTAATGTGGCTATTTAGGGATGAACAGCAGTATTTTCAG
AGAAAAGTATAGAAGAACAGATGAAATCACACAAGATGCACACATCTGGGTATTGGTGGGCATGTACTTTCAGCAGTGAGTTTACAAATAATCT
AGACAGAGTTAAGTATCAGGTTTAAGAGTCCTAGGATCTAAGCCTGCACGGGTTCAGTCTTCTGCAGGAACTTGGGAATGTTGGGGTTCACTTA
GGCCCCCAGTGTTGGCATGTACACCCACTTTCAGGAGGCTGGAGGTATAAGTTCCTCTCTGCATTTGTCTCAGGTCATCACAGGCCTCAGAAGT
TTCTCAACAGAAAAGGATTGCATGGGTTGGATGTCCAGAAGGATACGTGGAGTTAAACATCAGTCAGTGGAGCTAGAGGCTTGCTAAGACTTGG
GAAGAGGATGAGGGAAGGCAAAACCTGTCACCTAAAGGGGAATTCTAGAATGTACTTACTACACTCATGACATTACCCTACCACCTTCATTTAT
CACCTTGTGTCAAACATAGACTTCTTGGTCAAGACCACTATGCTGTAAGCTACCTGAGGGCAGGACTATGACTTATTCTCCTTTGTTTACCCTG
CCCCAGTGCCTAACAGTGTCTTTGGAATTGGAATGTTCAATAAGTGGGTTAAACTTTTGAAACTGAATATTTCAAATAATCACAGATAACAGAT
GGTTAAAAACCAAGTCCTTCTCAGCCTAACTAGTGCCAAACCAAATCATTGTCCATGTCTCCTAAAGAAATGAAAGTTTTCTAATTACCTATAT
AGGTCTGGACTCAAAACTATAAGGCAGCACACTGTCTTTGGTGTTGACCTATGTCCCAGGTTGTCCATGGTCATCACACACTAATGACCACTTG
TATGTATTTCATCTAGAGGATCACACAGAGTGGTATGCTAGGCCCAAGGAATAAGTTTACCTGCTTATTTCACTTCTCCACTGTCATCCTACTG
TGTTTCAGGAAGAATTTCTTTTACCTCTGCTTACTCTACTAAGATGCCCCACATAAGCCCCCAACTCCTTTTCTTGTCCCCCTCCCACTTTGTT
GTCTATTTTCTTCTGTCAGACCCCTTCCTCCTCCAAACTGAACCTTCCTGAGAATATACCAAAGAACTCAATAAACCATTCAGCCCACTATAGT
TCAATAAATGAGCACTTCCAGAATTTGCATTTTAATAGTGGTTCAACCTCAAGTCTCTGTTGGTGGAAATGTCACTGTGATCTAGGTGAGGGAA
ACATTTTGAGAGCCTGTGGGAACCTCCAATAGCACCTTATAGTAGCTTCTTCTGGTGCCTGTGTAATACTACAATTTCCAGAAGCCTTGTTGAT
ATCCAGCTAAGTGAAAAGAAAGTCCTCAGTTCCAGAGTTCTTACTTCTATTTCTTTTTTTTTTCTTTTCCCTTTTGTTTTTTTTTGAGACAGAG
TCTTTTTCTGTCACCCAGGCTGCAGTGCAGTGGCGCAATCTCGGCTCACTGCAACCTCTGCCTCCTGCGTTCAAGCGATTCTCTTGCCTCAGCC
TCCTGAGTAGCTGGAACAGATGTGCGTCACCAAGCCCAGCTAATTTTTTTTTTTTTTTTTTTTTTTTTAGTAGAGACAGGATTTCACCATGTTG
ACCAAGCTGGTCTCGAACTCCTGCCTATCCACCTGCCTCAGCCTCCCAAAGTGCTGCAATTACAGGCATGCGCCACCGTGCCCGGCCCTTACTT
CTATTTCTAAATAGCAATGGATTCCACTCTTCCAAGGACCCAGAACTTGCCCTAGGGGTGAGGGTCTTTTGTACATCTTGCAACTGAGAACTTT
TGCTAGAATCAGACAGACAACATAGATCTCAGCTCTAATTTTCCAGGAGTGAAGCTGTGGAGCAAGTGAGTTACCTCTCTGTCTCACTTTTTCA
TCTGTACAATGAGGCTACTAAAACATACATTCCAGGGTTGCTGGGAGAAAAAGACAAGACAAGGTATGTACAACACTTGGCACCAAGGAAGCAC
ACAATTCATGGGAGTAATTACTATCATCCTCACCAAGAGTTCATGATTTACTGGGGGACACCAACACATGTACAGTCAACAGGGTAAAATGCTA
AGGGTGTGGGGCAAGTGTTGAGTCTGAGCTCAGTAGAAATTTATGAGCCTCAAAGGCCCTAAAACATCCTTGACCACTCAGTTCTATTTCACAC
CCTTCTTGTACTTCACCAACAAACCAGCAACATGCTTCTGGTTGGCAGCCCTTATAAAAGAATTTACCTTCTATAGTCAAGTCCAAAGCTCTTA
ACTGCTTACCACATGACAGCCAATAAGACAAGATATTGGGGCAAGGAAGATGACTTTTTTTCAGGGAGCCAGCAATCCTAGAAGATGGCAGACT
GATGTACTAAAGAACCATCTTAAGTTGATAGATTGTTAGGCTCCTTTTATGTTAGGGGAAGGGGGAATAGGTAGGGGGTTAAGGTCCACAGTTG
ACTGGTGACCACAGGCATCTGGGCATCAGCAGGGATCTAGGAGGTTGCAAAACTTCTTTGTTCATGGTCAACTATTTTCCAGATTGACCTTGTT
TCTATAAATTTTTAAGATGGCATTGTTATTTGTGTGCATACTTTCTAATCTCCTCAGGGGTCTGTTTCAAAAGAGAGATGGTCATTAGTTCTGA
AGTTAAACTATAATCTAAATTCTTTCCATCATTAGCTTGGCCTATGTATAGGAATGAGCAAAGGCAGTTAGCCTGTGAGGTTAGAAGCAAGATG
GAGTCAGCTATGTGATATTTCTCCCACTGTTACACATCTCTGAATACAAGAGGAATACATTTCTTCTTTTTGCTGTGTTTGCTTCAAGCCTGTA
CCACTGAGAAATTGGGTGATCCAGGGAAGTTTTACTTCTCTTGAACCTCAGGCTCTATCTTTGTAACAATGGAGAAATCAATGCCTGCATTCTA
GGCCCTTTTGAGGGTGGAGGAAGACAATGGTTGAGAAACTTCTCATCTATAGTTGATCCTCAATGCGTTTGCCCCTTTTCCTGTGGCACTGCTT
TGTATTTGGATGGTTCGAGCAGTGCGTCACACATGGAGGAAAGGCAATGGAATGATGACAAATAGTTTGTTGATTTGGTGCTTTGTACATTCAC
CCACAAATGGCACAGTTTCAGGGCCACTGTCCTTGCCAAATACTATCTGAAGAGCTAGGTTATGAGCCTTTGGAACCCAGATCTGATAAGCATG
GAGGAAAACTCCTCAAGGGAGATGAAGCTAGGAGATTAGCAGAATATACTTTCTCCATTTGTCCCACATTTGATGCCAGCAGGCAAACCAGAGC
CAGCTGGGCAGTGTACAATTCCCAGAAATAAGGAAAGGGTAGAAGCACCTGTTTTTCCTCTAGCAAAACCAAGGCAAATGGAAAAGCTCAGAAA
TATATCAGCACCATTAGCAAGAGGCACAACGGGGAAGGCTGGAAATGTGCTGAGGAAAATCACATTGCCGGTCAACGCATAGCTACCTGCTGGA
GAAGAGAGTCTGCAGACACACCTCAGCCTGGACAAAGGCCTCCAGGACCGGGAGCTGTGCTCTGCAGGAACTGGGCCCCAGGCACTGAGGACTG
AAAACCAGCCCAACAAAGCATACCTGTTTCCATTTGTGAGAGATGAAATGATATGGGTTTTGTGACTCACTAAACCACTTGGAAGGCCAAACAA
TTACAAAAGCAAGATGCCTGGCTTCTTCTAGCCACATGGAGGGGCCCTCAGACTTGAGTAGCTGTGTGCCACTTGTCTGGGGTGGCTGCACCAA
AAGGAACAGTGAGGTTTAGATGCTTGTTATTGTTCATTGTTCAGTTTTGGTTCTGTCTATTTACTTCTAATTCCTCAAATATTATTGTGATTAG
TGGTATAGAAAGGAAACGTTCATTTGTGAGCAAAGTGTGTAATAAGAATTTAATAATGAAATTCCCAGAAAAAAAAACTCAGATAGCTAAAGAA
ACAGCAGTAACATGAGCTCCAAATATCTTGAGTTGTATTGTTAAGGTGAGAATCCATAATGTAAAGGGAATAATGTAAAGGGAATGCTTTGAAA
TCAAAGGCATAATCCAAGTGTGATAACCTAAAAGGTTGTGGATTTCTTTTAAAGTAGAAATTACATGTGGCTTTGGGAAATCATCAGCTGAAGC
ATAAGCTATGCTTATGGATTCTCGCTGTGTTGTCACTAAGACCGATTATATAAAGACTTGGCAAATCCATGTACAAATGTAAAACTTCCCACAA
TGGTATTTCATGGGGATCTCCTTTTACAAAGTGGTCGTTGATCAGGACATTTTTCACTGATATATTCCTTTGGAAGCAATGAATTTTAGATAGG
ATATGATTATTGTACAATGTGATAGGTAGCTCTGATTATTCAAGCAGCTAGGGCATTATTCAGTGGCAAAGCATTAAGCTACTCTGGAATCTTT
CTTCTGTGTAAGCAGGCAGCTGCTGTTGAGACTTTCTCTTTGTGGGTGAATTTGCCCAACCAATGGCAAGCATTCATTGGGAGTAGGGGAATGA
CGCTGATCACTGTTAATGACAGTTGAAAATGAAATGCTTTAATGAATCTCATACTTGGACTTGGCTGCTGAGCCAGAGCCCTGTACTTGAAGAG
CTTAATTATGATGCCTCAAAATTTAGATCTTGACCCTGAACTGCTATGACTTCCTCCCACCCTCAGTTTTGAGAGAAACTTTAAATTCGCTAGG
GGATTCAATGTTTTACATGCTTGGTTATGCAAAATGGGAACAGTAGTGTCCAAACAATGGAATAGGCTTCTATATTATTATTTTCATTTAGGCA
GTATAACTGACATGATTACCAAAGTCATGTTATAGTACATGTATACAAATTAATTTCATACCGGGGGACTTCACTGGGGGGAAAAAAGGGAAGT
CCCTATTTGTGAAAACTATGATTTTGGTTTCTTTCTGAGTAAAGCACTGCTAATCTCTTTGGTGTACAGGAAAGTATGGTATTATGTTCTAAAA
ATCAGAAGCCCCAGACCTTAATTTCAACTAATCTGATGACATCATTTTGTACTCAGAGTTTCATTTCCCAACAATTTCATGTTTCTTAGTAGGT
GGGGAACCCAGAATTGAAAGGCATCAAAAACAGCTAAATATAACCAAAAACCTGCTAACTCACCAGCTTTCTAAAATTCAGGAAGTATCTTTAA
CTTGATTGCTGAATCTCCAAAGACATGATGATCAGATGACTGGTTAATGGGTATTTTTTCAGACTCTACATGATTATCAGAAATGGCTGCCCTG
GCCAATTGTATACCTACGAAGAGGTATTATGTAGAAGAACTTATATTCATCTACTTAATGTGGACCTCAAATATTAAGGATGTTCCTAATGTGT
TCTAGACCGGAGTCAGCAATACTTTTCATAAAGAGCCAGATAGTAAATATTTCTAGGCTTTTTGGGCCATATAGCCTCTGTCACAATTCCTCAA
CTCTGCTCTTGTCACACAAAAGCAGCCATAGACAATGCATAGGTGAATGAATGTGGCTGTGTTTCAATAAAACTAGACTCTCTTAATGCCAAGT
ATTTGCTGGAATAACACAAATTAATTTATCCTCAAATCTGTTTAACCTATTAATATGTCAGTCAATATTACATTAATATTTTATGAATCTGGTA
TCATATTATACATGAATTTATCTGAACATTTGGTTAATTCATGAATATTTACTCAATATTAATACTACATCATGAATCTGTTATCAAGTCATAT
ACAGATTTCTTAAAAAATTTCCCTTATTTAGCATACTTGTGAATAACATCAATAAGCAAGTATCTTAAGTTAACTATCAGGTTATATAGCAGAT
CCTCTCCCCCTTTTGTAACTCTTTCTGTTACAAGGGATTTTACATTATTATGGTATTGGGAAGTAAGTCTATTTATCCTGTCCCTATCTGTCTT
CGTATTAACCATATTCTGTTATAGCTTTTATCTTTGCAGACAGAATAGTTCAAATCTTCATGATTTGTCCTTAAACAACCCCCACTTCCTCATC
CTCTTGAACATTTTATGCCTCCATCACTGTCTCTTCAGGTCTTGTGAGTCCTTGAAGTGCAGTGACTAGGCTGGAACATGGTGTTCCAGGTTGG
TCACATCACCTCTTCCTAATGATGCCCCATTGTACCTTTTGACTATAGCAAGGAATAAACAAAGTCAGAAGTGTGAAAGTGGGGGAGAAATTGA
GAAAGTGCAGGGGATTTAGGGGAGAGAAAAATGGACTACAGGAAAAGAAAACATTGACTTTTGCAAAGTGCTCTAAACTGGCTACAAATGCATT
CCGTAATTCTTTAAACCTTAATTGTAACCGCAGTTAAGCTTTCCGGTGTGTTGGAATAAGAACAAAACTAAAATCCTTCTTTGTATAGAAATTA
TCCTGCAGTAACTTAGAGCCAATATCTCTAGTTAGGATGAGCGCAGTGGCTCATGCCTGTAGTCCCAGCACTTTGGGAGGCTGAGGCAGGTGGA
TTGCTTGCACCCAAGTAGTTTGAGACCAGCCTGGGCAACATAGCGAAATGCTGTCTCTACAAAAAAATTAAAAAATTAGACGAGCATGGTGTCA
CGCACCTGTTGTCCTAGCTACTTGGGGGCACTGAGGGGAGAGAATCACTTGAACCTGTAAGGTCAAGGCTGCAGTGAGTCATAATCACGCCACT
GCACTCCAGCCTGGATAACAGAGTTAGACCTGTCTCAAAAAAAAAGAGGAAGAGATCTCTGGTTAGAGTTTGTCTCTGATGGATACTAACCATG
CAAGTAGTTTACACAAACCACATTCTTTCTGTAGAATGAGAAAATCTTGGATATAATTTAGCATAATGAGCTAAATAACTCTAGACTGAGCTTC
GCTGCCCAGACAGGACTCTACTTGTGATCTGGGAAGATCTACCCTTGCACTGAATTCTCTCCTCTTCTCTCCTGACTCTGTGAGAAAAGTTTGC
ATGAATTTACTGAGATTAGTTATAGAAGGAACTGTGTGTCATTGAAAGTCTTGTACAGAAGTTCACATTAAAATATATCATAGTCATCTTTGGG
AATGATTCTGCAAAATTCATAAAACAAAGATCTCTGAAACCCTAAGGCACACTGTGCTGGAGGCTAAGGGAATAATAGTGAGCTGAGTGGTTTC
TGCCTACACAGAGCTTCTAATATATTGAGGAAAGAGATCTCAATCAAATTCAATTGCATTTATAAGTATGGTAAGGACAACAAAGGAGAGAGAC
ATTATTCTGTAGGAGAATAACCCTGTCAAAGTGATGACTGATTTAAGATCAAAATGGGAAGGTAAAATTTTCTCAGTACCTGCAAAGGCACTGA
GGTGGGAGGGAGCATGCCAATGTAGGGAAATGAAGAAACCCAGTGTGTATGAGCCAAGCTGAATAAAACATGAGAAGAAGCTGGAGAATGAGAG
AGACCAGTCCCCAAGCTCTCAAGGAGCAAGAGGAAGCCTTTTCGGCATTTGAAGTGGAGGGATGGCATGATCTCGTGCGTAGTTTTTAAAAGAA
CCACTCAGGCTACTCTTTTGAGAATATTGTGGGAGGCCAGAGTAGATGCCTATAGACTTATACAGAGGTTAAGGCAGTTGATAAGAGTATAAAC
AATGATGGTAGCTTACCTCACATGCTTTTTAGATTATAGGCTAGAAGGTGGCCCAAGCCCATATACCATCATGGCACAGTGAATACGTTTTCCT
TCTGGATCGTGGAAATGAATATTGTGCCACACAGCTTGATTGACACTCCCCAATGACTTTTAATACTGTGGGATCTCTGGCACATACTTCAGAA
GATGAGAAAGGAGTTTTAATTAACCTCTGTTTAGGAATTCCATAGACATAATTTTCATTAACATCTACTTGGTATTTCATCTCCATGAATTCTT
TCCAGTGATAAAGCTAGTTGGTTTATCCTCCAGGACTGCTCAGTTCTGAACAGTGGGTCTTAAGTACTCTTTGGATTTCTTTGAAGGCTTTTTT
TTATTTTTGTTGACTACCTCTCTACTCATTTTGCCCCAGAAAAAATGTAGTCATTTCTTTGATAAGTGAAATCTAAGAAAGGCTTATTTCATTT
GGTACATTAAAGAGATGGAAGAAAGTAACTTTACATAAGAATATAAATGCCAGCTGTCCAGCATATTACAGAATAGATTTCTAAATTTGCTGAA
AGGTTAGGAAAATGGATTCTTATATAGGCTTAAAATTTTTATAATTTTTTTGGCATTCTAGAATGAAATAAAGAAAATCGGTATCATCTTGATC
TCATCACCCAGAAGAAAAAAAAAACACTGATACATGGTGAAATATTTTCCTCTCTCATTTTTAAAAAGAATCAGGAATAGATTGTATACATATT
TTTCAAATTCTTTCTACTTAGTGTTATATGATAGTCAATAATATATGTGTATGTAAGTAGCAATAAATCATTCCTGAAAACTTGTTTTTTAAGC
TTGTATAACATTCCGTCATATGGATGTACTGATTTTGCTAATTTGTTAAGTTTAAAATAATATTCATTTTCATTTCTTTGACTACTAGGTATAA
TTAATTTTTTCATACACTCACTACCCTTCATACTTCTTTTATTGATTTCCTTTTTATGTTAATTATTAAGATTTCTATTTGACTCTCAACATTT
TCTTAATTTTTTTATCATTAATAGATGTAAAAAAATTCTTAATTTTTAAAAAATAAACCCTTTTCATCTTAAAGATAGCAATCTATTTTCCATA
ATACTTGTTATAAATATTTCCCCAGTTTGATGTTGGTCTTTTAATTTTGTTTAAAGTATTTTTGACAAATAAATAAGTTTTTGTGGCTTGAAGT
TTGTTCATTTTTTTCATTTGTGATTACAGTTTAGTTGTTGATTCAACACATCCTTATTGAGTGTTTTGTGTTCAGCACTAAGCGAGACACAAGA
GGTAAGACAGAAAAACAAGGTGCAGGTGGTTTTGCCACTTAGAGATTACAATTTAATGAGGGGATAGACAAACAAAACCAGGTCATTATAAAAT
AGTATACTACAAGCTATGCAAGAGGAAATACAGGTGGGAAGAAGTACCTGATCTGGACCTGGGGGATAAGGGAAGGCTTCTGGAAGGAAACAAC
CTTCTAGCTGAGAATGAAGGAAAATGGCAACATGAATTTAGAGAGAGGAAACAGTATGTGTGAAAGTGTTGTTCAGTGTCCTAAATGTTAAAGG
TCCTCTTTCCCCAGAACATGGAGTGTGTGTGTGTGTGTGTGTGTGTGTGCGCGCGCGTGGGTGTGTGAAGGAGCAGGGCAGGGTGGTGGTCAAG
TGTGAGCAGGAGGCCACTGGGGAGGCTGCGGCAGTCCGAGAGAAAGACAATGATGTTGGGCAGAGGATGTGTTGAATAACTCATCCCTTTTCAC
TGGACAAGCAATGCTTCAGTGTATATTAAGTTCTTATTCATAGCATACCGTCTATTCCAGAATAATCTAATTAGTTTTATTGCTATATCAATTC
TTGGGCCAACACCACACTGGTTTTGAGTTTAATGAAACGTCTAAACTGGTAATGCATGCATCCTCTCATCACTTTTCATTTTCAAAATTGATTT
ACCTATTCTTGCCAATGCGTCCTCCCAAGTAAACTCTAAAATAATTTCAAAAGTCACAAAAGCATACGTGTGTGTATATGCATGTGTGTAAATA
CATGTATATATGTGTATGTGTGTTTCTTCCAACCATTTTTCAGTTTTTGGAGAATAAGATGTTTTTATTCAAGTTGCTGTCAGTAAATATTTTA
TATTACATGACTTCTCTATACACACACACACACACACACACACACACACACAATCTCCCATTGGTAATTTGATTGGGAATGTGTTAAATCAGGA
GAACAGACATTCTTTTTAATATTGAGTCAGCCCATCAAGAAGCATTGAATGTTTTCCCACTTACTCAATTCTTTTATACTTCTTTTTAAGCCTT
TATAGTCTTACTCATATAGGTCCTGCATATTTATTGACGGGGTATTCTTTTTTCTTTTATAATTTTAGCTTTTAATTATGAATGGATTTTTCTT
CCATTATCTCCTCTCTGGTATTACTGGACTACAGGATAAATATTAATGATGTATATTTACTTTTTATCTGATTATTTAACTGATTATAATTAAT
TATAAGTGTTCATAGTTAATTATAAAAGTTTATAATCATGGTGTCTTTAAATAATGATAATTGTTCTCCTTCCAAAAATTGCATCTAATTTTTA
TTTGGTGTCTTACAGCATTGATCGTAACTTCTAATACTAAATAACAATGATAATAGTGAGCGTCCTGTCTTAATATCAATTTCAAGAAGAACAC
CTCTTACATAACATTTTTAAAAGTGTGTTATGTGGAAGCTAATAAAATAAACTTGGGAAATGCGACAAATGAAAATTCTCTCTCCCAGGCATTC
TTAATTCATATTAACATATTAAGATGAGATCTGCAACAAAGAGATGAGTTCAACTGAGTTTAACCCAGTATTTCTAAAATGTATTTGACTATGG
AAGTATTTTTCCCTCATTTAGAATTTTACATCAGTTATGACAGTCAAAATTATATTAATTTGATCTTCTTTCTTTTTTTAGAAAAATAATTTTC
ATACTTTCTTGTCTTTTCTGTTTGATCTCTCTTTTGCCATATGGACTATGATTTGGGTTTACTTTTAATTTTTTTCAATAATTGAGAAGCTCGT
CACCCTTCTTTTTATTTCTACAAGTGGTTACACTTAAAGTTTCCAAGACATTTCTATCCATATCTTATACTTAGCAAAGTCAAAGACATGACCA
TACAGTTTTCTAATCAACTTCAAAATGAAATGTTATCTTTGAATAACCCCATCAAAAGAAATTTAATCCATTTTTACTTCCTTCCAACCTTTCC
TCAGTATTGGTAAGAATAATTTAACATTTTTTATTCATGTTACTGTCAGTAAATATTTTATATTATATGACTTCTCTTTTGTGAATTATGTGAT
CTGTTCAGCAACATAATCCTTTACAATACATTCCTGAGCAGTTCTATGGTTATCTGATTTCTGTGTCCATAACTATTGTCTTTTTATACCATAA
ATTTCTCATTTTTTTACTTTTTAATTTTAATATCTTAAGATTCGCCACCTTTTATCCCATTTATCCTTTTAGGGTGTATAATGTAGGTCATCTA
CATTTTGAGAAGTGCCTGATTCTGTTTACTATAGTGTCAGTTTTACCCCTTACTGCCTTCAGTGGACAAATTAGTACTATTATTTCATTTCTGG
CTCCTCTACAACCCCTTTTAATCTAACCCAGCACTCTTTTCCTTCCTTACTTTTATCTCATAATGGCTACCTGTCCGTTTTCCACAGTATTTTC
TATTTCAGTTGAAGATGATAAACAGTTTTCTGACATTGACATTAAGATGATGTTTCAGTTTTCTTGTTAGACAATTTTGTTCACTGTCTAAGCT
ACTTATTTTTCATGATTACTCGTCTAGGAATAAAAATCAATCTACTTAGACCCAATATTTTTGAACACTGAGCATATGTAATGTGTTCTTAAAC
CTGTTCAATATGTACTGGGCTCTAAATGTCCTGGCCTACAGCTGGAGGGCAGAATGACATGTCCTAGTTCAATATCAGCCTCTAACAGGAATGC
TACTGATGTAGTTTTCTGCACTGAGATAGGATCTTCTTTTCTTCCATTGCCTATTCTCCTGGAGGCAAAATAGAGAACATAAAAAGTCATCATC
TTTGGTATGTTCTTCTTTAGGGGCTTTTTCTTTCTCCCAGCCCAACCCCCATAGTCCTTACTTGCTAGATGTGTGTTCCAGGATTCAGTTCACC
ACTGCCCAGTCTTCTAACATCCAATTTCATTGTTTTCTGAGAGTTGAGAATTTTCATACAGATATAGAAGGGATGCTGTGCTTGTTTGAAATGG
TTCTATTTGGCAGAGGATAGCCGAACAGTTTTTAGGCAAAGAATGAACTGTCTACTTACTTTCTTTGAAGAAGCCAACAATTTGGTTTTGTTGC
CAACAGCAAATAATATATTGGCTAATAATTGTGGTTTACAGTGACATGGGTAATGTTAGTCATAACATGTCTTCCAAGTTTCTGGCTATGGACT
AACTGTAGGTCTTGGAGTTTGGTATTATCTAGAATTTTCCTTCTCCCTCCTCCCCCACCTCTTATCTTTTAGCATAGTTGAAGATTGGAACTTC
TTCCCAGAAATCTAGCATCTTCATAAGTCCTTTCCGGTTTTAAGATTTTTATCTTATATTTTATATATATATTACATATATTTCCTGTGAATCT
TGGCTGAATTTCTCATTTAATAAAGTCCAAGTTAGTATGAATAAATAACATGTTTCCCGATTTTACACACATACAGAAATTCAAACACATAATG
AACTCTCTGGCAGCCTTTAGCATTTGTAAGTGCTCTCTCACCAGGAAAATCCTTTAATAGATAACCAAGTGTCCTGGGGAAGAAGGGTGTCAGG
AAACTTATTATATTTGTGTCACTTTCTTCAAAGGGTGATTAAGTATATGTCTCCAAAGATGTGATGTTATCATCTTTGAGTATCACTTGAATTC
AAGTCAATATTTATTGAGACCCCATGTTGTATCAGGTGCCTTACTAGAAAGTCGACATATAGAGATGCATAAGACATGATCTTTTTCAGTGGGT
AATTTAAGGTCTTTCTTCTACTGCTAGCATTGTCATGTGTAAGGCTGGTGGGTTGAGTCTTTGGAATTTGTGAAGATTCTTCCAGGTTGTTTAA
AAAATCTTTTGATTCTCACAAATGTATTTTTTCTATTTCTTAGAATTATGTTCTTTATGGCTACCAGGAAATCCTTTGAAACTGGAATTAGATT
GCATTATTCCACTGGGGTAATAGGATTGGTAAGGTAGGAGTGTCTTTGAAGAATAATGCTAAATAACCCAGACACTAAGGCTGAATCTTCAGCA
CAGCTCTTAAGGCATCCTGTAAGTACCCTCTGCTGCTTATTAGAAATCCCAAAGCCCTGCTCCCTTAGGAAAGCGGCCCATCCCCATCCTCATC
ATAAGATGCAAGGCATAGCTAAACATCTCAACTTCTCACTTGAGTATTATATACTCTCAGGCCAGGATACTTTCCAACTCAAGAGTAAGATAAC
ATTACATTCTGATCTTCTTGTGTGTGTTTTTATGTTTTTTTTTTCATTTTATTTATTATTTTTTTTTTTGAGACAGAAATTTTACTCTTGTCAC
CCAGGCTGGAATACAACGGCGCAACATTCTGATCTTCTTTTGAGAGAAAAGTTTCACACAATTAGCCTTTGAAATACACATTCCTTTAAGTACC
ATGAAAACATTGTCCCATACAACAATATTTCTAAGAAACACACATGTCCCTCCCACAATGATTTTTTCTTGTTACCACCAATAAAAATTAGAGC
TGTGTTGTTTTTGTTGTTGGGTTTGTTTTTGTTTTTGTTTTTTGGTGACTACTGTGACCCTCAAAAAATCAATCCGACTTTCTCGATCTTGATT
TGCAACACCAAGGTTACCAAACAAAGAGAATGGTTCAATGAGGGAAAGCTCAACTACCTGCCCTAAATATTTAGTATAGCAATGCTGAGCTTTT
GGTAATGGCTCAAAATTCTCATGCTTTCCCACTAAATGAAAAGGGGAGCTTTGCTACTAGTAAACCATATTCAAGAAATAATTCTTATTAATAC
TTTGCTTCTTTCCTATGAATTATTGCTTTGTAATGGATGTTTGACAAGTTACTAGCAAAGCAGTCTTCGATGAGCACAGAGACCCATCAGATCT
GTATTAACACCTCCAACCACTGGAGTTTCATTAAGTAGGTTTCTCTACCCCTCTCCTTCAGGCCACAGATAGGAAGAATGACATTGGGTCCAAA
GTTCTGGCACAAAGAATGATGTCTGCCTTCCTGTGATGATTTCAAATTCAGGGTAATAATTCCAACTATCTTTCCCTGGCCATGATAGAAAGCT
CAAGAAATCTTGGTTCCTTGTCTCTTTTCTTCCATCCAGTTGGTCTTTCTAATTACACACTCAACACAATGTTCTGCTGAGCAAGACATCTGCT
GTAGCCACTATCACCCTTGGCATAACCAGATATTTAAAGGTACCTAAAGGAGTTTAGCTATATTTTGCCAAGATCCTAAGTTCTATGGCATAAG
AATCCATCCACTTCTCTATTGCCTATAGAAAAGGACAGCTCAGTCTTTCTGAAAGAATTGTTCTCAAACAAGAAAGCATTTGAGGTAACATGTC
AACAAGGTGGACTGGTAATTGGTGTTCCTTTCAGAGCCAGTTGGAGGAAATGGCCAGCTCAGACATTCTCTTCTCCCCAGTGTCCTATGGGAAC
AAGATTTACCCAGACACAGCTGACTATGAGAAGTCTTCAGGGATTTACAACCTGTCTAAGAACAAGGCCAATGGGGGACTCACTTTGCAACACG
TCCCTATTTGATTGTCAGAGGACAGATACTCTATTAACAAAGGAATTTGCACTGGTATTTTCAGGGACACATCAAGTAATTGCCACTTGAACGT
TTTTCCAGGCTTTCAAAGGAATGTAAGAATGGCCTAAGGCTAAATTACATTCATGTCTTTAGTGAGTCATTATATTACTGGCAATAAAGTATAT
TATTACTTTACTTTCAGATGCCCTCACATATTTTTTTAAAGTAGAAAGGAGACATATTATGGTCACCATTCTTAAGAGACTTAACTAAAAGACA
CTAATTTAATCTTTTAGTTACTTAAAAATAGCTGTTGCTCACCCACTATGTGCCAGGCTCTATACCAGGCACCAATATGAAAAGGTGAGTATTC
TCTGGTCTCCCTAAGCACAGTCTAGTGGAGGAGACAGACAAAATGCAGCCATGGCCAGAAGGGGGAGCAGTTAATGAACAAGTAGGCAGAGAGT
AGGGAGGGGATTCTGAGAGACAGTACGGACGGACTGAGGGTAAGTTGTACATCCCTGATATTTTGGGCAATTGCAAATAGTTTGATGACCAAAT
CACAAGGAGAAGAAGTGACAGCAAGCATGGCTGAAAATGTAGGTTGGTGTCAAATTGTAAAAAGCCTTGAATACTGTGCTGAACGGTTTGATCC
GTGAATCAGGTAGAGGACTTGACTCAGGTCTCTTGACTCAAAGGTCATGTTTTTTCTCTGCTAGATTGCACTTATCCTTCCAAATGCTAGTGGA
CACCACACTGAATTTGATCAAAGATCTCTCAATGGAAATATGTAAATTTCCTGTAAGAAGCAATTGGTAGGTTCTCTTATTATCATCTCCATCT
CACAGATAAGGAAACTGAAGTATAAAGAACTTGAGTAACTTGTCCAAGGTCACACAGCTAGTTAGGTGATCCCAGACAATTTGGCTCCAGAGTC
ACTAGTCCTAACTACAACAATTTAGCCTCTCTTTATCTAGAGGTCTTTATGCATTTGTCTGCATTCTACTTACGTTGGTATTGGTGAGTAAAGA
TTCCCTTCCAGAATAGGTTTGATAAATTGCCAAGTCTGAAACTGAAAAATTAATATCCATGATAACAGAGGTTTTGGGGTCAAGGAGTGAAAGG
AATTCATCCCATCCTGTCTCTAAATACTTTTTTCTTTACCAATTTATGGGAATTTGAGACCAGATATCTGGAACCAAGCAGCCTCACCCTATTA
AGGGAAAGAAAAAGAACAGAAGAAAAGAGGAGAGGAGAAGAGAAGAGAAGAGAACTAAAAGTATCTGCAAAAATATTAAAGGATTTGACCGAGA
TCTCTGAGATGCTCCAAAGATTGCAATTGTGCTTACTATCCAAGTTAATGGCTGTAAATCTAAGAGAATTATGATGTGCCTGGTAAATATTGGT
TGTTTTGTTTTAAGTCTTCATGGAGAAAATGGAAAAGACATTGGCATGTAGGATGAAGTCACAGCAGGACAGACAGGTCCTGCAAGTGTTTCTT
TAGATGCATTGATATTCATAACATCCAAACAGGCCTTAAGGCTGTCTTCTTACTTTACACTTACCAAAGGGGTAAAATATCATTTTCCTTGGAC
ACACAAATAATAAACGTACTCTCACAACATCACTTGAATGTTGTGGTTTTGCAAATATACTATGTTTTAAAAGCTATAGCCCTCAAATCCCTTA
CTAGACATCAATATTTGTTGGAATTTCAACATCAAAGATTTATTGTGAAGGGGGAGGGGGAGGAGTTACTCCAAAGGCTAGAACCAGTGCATAT
ACCATAATAACAACTCTGCATGCATTATTGCTTCTAATCTTCATAATTTTGAGTACTATCTTTATTGTCACCATTTAATGAAGATGTTAAGGTT
CTGGGGGCTTAAGTGACTTGCCCAAAGTTACACAGCTTGGTAGAAACAGAATTTACCACAGAACTTGTGTGTTAAGCCAAATAATTTTCTAGTT
CCATACAACTAAAAATTGGAAGGAAAAAGTAGAGCCTTATGAATAGAGTCCTGAGCAAGAAGGATGAACAATCTGCAGATGAAGTACTCTGTAA
AGGAAAGATCTGGGTTTGAATTCTAACCCTGCCTCCGTTCATCTGTGTGATCTTCGCAAAGTCTGTTAACTCTTTAAACTGTTGGATATTTATA
AAATGGGACTGGTGCTATTTGGCCTGATATCTTACAGGGGAAGATTCAGTCAGGCCAGGCGTATGTAGGGTCTCTCTGACCTGGAAAGCCCTAT
ATAAAGACATGGTACCATTCTTAGTGTTAGGACTGAAACTACATCCCTAGTGTCCTGACACCTCATACCAATACCCAGAAACCCTAGGCTCTTC
CTTCAATCCCATACTCCTTCCTCAGCTGCCAGGACAGCTTGGTTAAGAAAAGCCTTGTTCTTGGGGAGTATATCCATGAAAGTATCTCTATTTC
GTTGCAGTTTCACATTAAGTAAAATTAGAGTGCCCTTCACGAAGAATATATAATTGAAAGCCCTCCCACTGACTGCTGGACAAAGCACTTGTCA
GTCAACAAACCAGGTAGATTGAGTCTGCCCTCAGAGTTTTTGTATTCTGAGAGTGACTAGCAACTTTCTCTCATCCTAGAGCAACAGGGCTTTC
AGAGGACAGGTTCTGGGGCAGCGTGTAGCCACGATAGAATTGGTTTGCTTTTGTGTCCAAAGTCATGATTATCAGGAGAGCTGCCATTTATCAA
CCACCTCTTTGATACTTGGTGTATAGGACAGGCGTGTTAACTATATTATTTCTAAACCACAAGATAAGTCTGTGAAGTATTTTTATCCCTGGTT
TACAAAGGAAGAAATGAGGCTCTGCGAGACATGCCAGAAAGAACATGCTCTACACAACCAACTGGTTACAATGTGCGGCTTTGTCCAGCACTAC
CTCTTCTTTCTTGGCAATCATCAGAGCCACTCAAGGCTTGGAGGACTGTGCTGGCAAAAAAGAAATCTTGTATTTTCTAAAACTCACACCCACA
CTTTGGAGGGGACAAGATTGAGATCAACCCCATTTCCTACTCTTTTCTTTATGACAGTTTATCAGAGCATTTTTTCCTTATATTCACTCAGTCC
TGAGTTTCTATTTAGTTGTGATAATAGATACATTTTTGAATTTTTAATACCTTCGGGTGCAAATTTGGAATCTATGGAGGATAAGGGAGTAACA
AAATGTTATGGAAATTGAAATATATAAATTATTAATTTTCCCTCTCCTCTACAGATTTACAATCACTTTATTTGGGGATATCAAGTAGGTTATG
TGGTTACTGTAGTACTCATTTAATAATCAGATTTTATTTAGAGAACTATAAAAGGTACAACTTAAGGGGGAAATATATTTTAGATGATGTTTTT
TTTTCAATTTCCAATTTTTGAAGAATGACGTGTTTTTAAAAACTTTTAAAGAATTTCTCCCACATTTCACTCCCTTTAAGGTAAATTGATAGCT
GTGAAAATGGCACCTTGGAAACATTTCTGTCATAATCATAGGAGAGAGTGAGAATTATTAATATTCTCAGCCATGTAGCATCATAAAGTAGATC
ACAGCTGTTTCTTTCAGAAAATGTCATTTGATTTTCGATCAGATGTATGGTCCATCTCACAATCTAACCGCTTCCACATCTGGAATATCTTAAC
TGAAAATGTCCACAGCATTACACGGCCTGGCTCAGGGGGAGAAAAGAACTAGGGCCTGCATCGAATTCTGTCTCTCACTCAGCTGATCTGCATT
CCTCCTATAGCATTTCCTTATAATTAAGAAGAGCTCATAGATTTGGGAACTGCATGAGCCTCTGTAACCTTCTGGTGAGGGTGAGAAGCTCAGA
ACTGCATTTCTATGCATTTTTCCCTAGTCTTTCCTGCTTCGGAGGAGAAGCCCTGCAAGAGCTTCCTGCCAAGGGACCCTGCTTTCTCAGCAGC
CGGATCCGGAGAGCCAGAAATCCATCTTCAGCTCCTTTGAACTTGGCTGTTGTTAATCATGCTGTGGTTGTCCTTTAAGAGCAAAGGCTTCACG
GAGAGCAGATCAGCTGGCTGTAGGGAGGGTTTCAGCTTCTCATTGTAGGCAGATCATCTGCTAACAGCTTACTGAGAGAAAGTTGAGGACATGT
GGCTGAAACACTGTCAGAGCTGCTGTGAACACAGTCCAAGTGAGCAATTTTATGACCCAAACCAAGCAGACACTAAGTGCTTTCTAGTGGTGGA
AAAGGAGCAGAGAGAAAAAGAACAAAGACCAAGTGGTGGAAACAAAAAAAATATATATTTCTTTCAGCAGCTAACATATTTTTCTGTTTAGAAG
ATTGGTATCTCCTACTTAACCACAAACTACAACACATAAGATCAAATAAGATTAAATGTCCGGTTTGCTAAATAGCAGAGCGAAGAAACTAAGG
TCAAGATTTCAGACTCTTACATCTGAGGAAAGAGAGAGGCAAAGAGAAGATACTGGAAATGCCAATATCCAGTCAATGAAATAAACAGAGGCTA
CTGTAGGCATCCCCAGAAGCTTGCCTTTTGTCCAGTGCCATGGGAACCACAGAAGGCACTCAGCTTTGCAGCTTCACTCAGAATTGGGGCAATG
CTCCACAGAGTCAAGGGCATGTTATTATAAAGTGCTCCATTTGTTTACTTTGGCAAGTGGAAGATGACTTTGCTAAGCGATACTCTGCTGGGGC
TCAGGGCCAGCACTGCTGGAGTAGGAAAAGGGAGACTTCCTGGGGCAAGGCTTCTCAATCTTCCTGCCAGCAGCATTGCAGTCGGAGCGGCACT
GAGGTGTCAACAAAAGGATGATGAACTGTGTCAGCAGTGTTAGCAAATGGCTGAAGTAGAACGGAGCTGCCCAGGATGCTGCCCTGTCGCTGGG
GAATCTGAGGGAGGCTGAAAGATATTGTAAAAGAGATTGAAGGGCTGGACTGGCTGGAGGAATGGTGCCATCGGCATCCATGCCACAATACTCC
AGGACTGCCCTGCCTTTGTCAGGACAGCCATCAGAGGCGGTTTTCCCTCTAACCAGGATGGGCATCACTCCTTGGGCTTGCCAGTCTTACCTCT
CCTCATGCAATTGCATGTCCAAGGCATGCCCTCAGGGGATGGTGCTGATCCAGGGTGGCAAACCCAAGTGGGGAGAAAATGCTGAATCTCATCC
TCTCAGAGATCTGCTGCAGTCAGCGACTATACTGAGGTGCATGATCTGAGCAGCAGTCGCTGGTTGGGGAGGCTTCTTTGTGGAGTGGGTCTCA
GCCACAGCCTGAGGGTCCTCTCTTGGGGATGCTACCAGTTAATTGGGTCATCTTCAGCCTCCACCCAAACTTAGGCCTGTAGGTCCATCAATTA
CTATATCAGCCTCAAGGACTGAACAGGTGGGAGGAAGGACTAGTGTGAGTGATGGAAGCAGCTGAGCTGAGGAGTGCTGGCACCTGTGCAAATC
CAGCAAGAATGACATCTGGCTGAGCTTCAGTCATTCACCTAAGCACCAATCGGGGGTCACTCCCAGATCTGAGCAATGAGGGAAACTTCCCAGG
GGGAACCCCGCACTTGGGTAGACCTGGGGGAGCAGCCAGAAGGAAAGGGGTTGAACCTGTAGTTGATTAAGATTCAGAGAAAGAAAAAACAAAA
ACAGAAAACGATCATAGTTTCAGACACTCCCAATGAGCCTGTGACCTCCCCAAAATCCTATAGCCAAACACACGGGTGGATCTGGCCTCACTTT
CCCATTCTCCCACTTGTGACTTTTGATTCCACATCATAGCTAAGGTCCTCGGCAAATTGGATTAAACTTTGGATTTAAAAATAAAATAGAATTA
AAATAAAATAAAAAAGTTGGGGACAGGAGAAAGTGGTTTCCAAGAACCTACACCTCTCTACCCTGAAATGACTGAATATTGTGTTCCCCTCAAA
ATTCATATGTTGAAATCCTAACCCCCAATATAATGGAGCCTTTGGGAGGCTTCATGAATGAGATTAGTGCGCTTACAAAAGGGTCCCAGAGAGC
TCTCTCACCTTTTTCCACCATGTGAGGTTACAACAGGAAGTCAGCAGTGTGCAGTCCAGAAAAGAGCCCTAACCAGAACCTGACCATGCTGGCA
ACCTGATCTCAGACTTCCAGCCTCCAGAACTGTGAGAAATTAAGTTATGTGGTTTATAAGCCACTCCGTCTAAAGCACTTTATTATAGCAGCCT
GAACTGATTAAGACACTCCTGAGAACGATTCCTTCGGGGAAGGTCTTGATTAGCCTTCACCTAGCCCAGTTTGCAAACACACTGGAGTGTCCTG
CCCATGGCAATGTTCATGTAGCAGCCTGGGAGACAGAGGAGGGGGATTCCTGCAGGAGCAATCTTCCCAAGGAGGCAGTTCCTACTAGCTAACT
TTCTTGTCCTGCTTGTGCTCATTGAAGGTCCAAACCTGTCCCCTGCCCTATGTTTGTTTTCCTCCTAATCCCCTGGCTGATGGAGTCAGGCTTT
GGAGAATTCCTTTAAGAGTCTTAGTTGGCCTTATGGATCCCAAAACACACCCTTCTCTATTGATGCCAGATTCTGAGCAGGCTGACAGTGCATG
CATGAGAACCTTTCATAAAATAGGACTATTTTTTATTCCCTTTGCTTTTTCTTTGTGTTATTTCCCCTCTTCACTCCCTGTCCTTTTACCTTAC
CATATACATTTGTCACCTCCTTGCCTGGAAAGCCTAGTGTCACCTCCCTGCCTTTTCACAGCCTATAGGATTGAGTCCAAACTTGCCAGCTTGG
CCTTTCATGGAGTGCAGGTTTAGTACAGGACTGCTAAACACAGTTTCAGAATGAACCATTCTCCTGGCCTTTATCATTGCCAATATTTCTCTTT
ATTTTATTTTATTTTATTTTATTTAGGGAGAGCCGATATTTCATAAGAGTTGATGGACTGCTTTGCCTGGAAACTTGAGAATTCATATTTTTAT
TCAAAATCGTTTTCATTTTTTTGCTTAGAGCCTGTTAAAGACTGAGGCCCTCTCTCTGTCCCTTCTACTCCCAGCCAAACCCAATAGACACATG
TGAGCAAGTTGTCACCTGACCCGAAGCTCCAGCCCCTGGAACCAACTGAAACAAAATGTATCCTTGCATTTTCCAGCAAGGAATTGCCTTGCCT
ACAAAAATACTCTGTACTTTAATATGCAGATCAATTATTAACAGTAGGCTGTACCTTTCACCAGGTTGTAAAAGGATGACTGCGTAGAGCCACA
GCATTCTCCTTGTCAGTGTAGGAAATGCAGAGGAGGATGTTACCTCCGGCACTTGCCTCTAAGACACCTAATAAAGATGAGGAAGGCTCCAGCC
TGAAGCGTGGGTGAGGTGTCCAGCTGCCCAAAGGCGACAGGTGAGATACTCAGGTCCCCTTCCTCTTTTTGCTGTCAGTTCTGATTCTCTCCTT
TCAATCTTTCTCATCTGTGATTGGGAATGGCAGGGGTGGGGTGTGTTTGGGGCATCAGTGGTGGTAAAAATGGTCTTATGGGCAGGGCCATGTT
GAATAAGAGATGAGCATGGAGACAGCGTATTAACTTAAAAGGCCAACACCGAGGCTCATTTGAGACCGTAGGAATTCCAGGGAGACAGTGTTGT
TCAGGCATGGGATCGAAGATCAGGAGGGCGGCTCAATAACACGCTGGCCCGGTCACATGACAGACCAGTCCTTGGAGTGAACTGGGAGACAAGG
AGGCTGCTTTGGCATGGAGCTGAGAACATCTCTAGATCGCACTGGAAGGCAGAGAGAGGCCAGACAGGGAACTCTGAAGAGCTGCCACAGCCTG
TGCCAGGGTGACATCAGGGTGAGTTGAGATGATTTTTTTAACAAACTTCATTTTTTGCAGCAGTCTTAGGCTTACAGAAAAATTGAACAGAAAG
TACAGACAGTTCCCACATACCCGTTTTCCCCCACAGAGTTTCTTCTATTAATAACATCTTATGTTCGTGTGGTATGTTTATCATGATGAACCGA
TGTTGATATATCATTATTAACTAACAAGTCCAGAGTTTACATTAGGGTTTACTTTTTGGGTCGTATGGTTCTATGAGTTTTAACAAATGTATAA
TGACCTGTATCTATCATTACAGTATCATACAGAATAGTTTCACTTCCCTAAAAATCACCCTCCCTTCCTTCCTGCAAATCTCTGACAACTGCTG
ATCTTTTTACTGTCTCTATACTTGTGTCTTTTCTGAAATGTCATGTAGTCAGAATCATGCAGAATGTAGTCTTTTCTGACTGCTTCTTTCACTT
AGCAATATGCTTTTAAGCTTCCTCCATGTTTTTCCAGGGCTTGGTAGCTTCTCTCCGTTTTAGCACTGAGTGATACTCCATTGTATGGATGTAC
TGTAGTCTGTTTATCCATTTGGGCAGACTTTCCAGCAAGATAGAAGGCAGAAGCAATGGAAAGGGTGGATGAAGGGAAAACTGCAAGGAACACA
GGACAAGATAGAAGCAAGAGCAGGAAGGTTTTGGAGAAGTGGAAAAACAAGCTAGAGAGATAGGCACAGAGGCCTAACCTGGGGTAAGGCAGGG
CCTTGTAACCAACGCTCAGTGAGAGAGGCAGCCCCTCACTGACCTGCACCAGGCCTTTCCTGATTTTATAGACAGAGTGTCATGAGGTCATGAG
GGCAGGAACCATGTCAGCTTCATCCTGAGAGCCCAGCACTTTGTAGGCATCTAATAAATATTTATTACACCAATGAAACCTTCAAAGTCTCTTC
TAAAAAAGCAAGTTCTCCCAGACACCAGAAGACAAATGGCCTAGCAGCCCCTGAGCCAGTCATCTCGGGCAGTTGTGAGCTGGTTGCTTATTTG
AACCCTCCATGCCACGGGGCAGCATCAAGCTTGCCTGACACACACCTCCTGAGCCAGGAATTCACCACTTTGCCACCATCTCTGCCTGGCTTGG
GAGATGCTCCGTGGACAGAACCAGAGCCGTGCTGCAGGGAAGCAGAAAAACCAAAGGAGATGCATGCCAATCTAACATGCCAGTTTAAAAAAAG
GCATTCTTGCCTCAGAGTAAGAATCCAGCTCTCCATGCACTCGTCTTTCAACCCAATCACAAGGCATCAGAATTAGCCCATATGACCTCTATGG
GACAAGAAGAGCAAGCAGGGTGATTTGGGTGCTAAGAAATAGCTGTGAGAGGATGATGCGGCAGCTGTGCCCTCCAGATGTTCGGAGAGAACAT
CTGTGCAACAATAGCAGTAGCTCCCATGCACTGATGTGAGAACCCAGCCTAAGGGGTACAGCAATCTGGGTGGTAGGGGGTAGCTCTCAGAGGG
TCTGACAATTCACCTAAAGATTGCAGGACCAATAGGAACCTGCCAGTCAGAATTTCCTAGCTCAGTGGTGCCCTCCAAGCAAGAGAGCTTATCA
GCCAGACCTGTGTCTTGGTGCAACATCACTGCTTAAAATCAAGTCCTCCCAAGGCATGGTGTCCATTTTACTTTCTCTGGATGAAATGAAGACA
GAGAATGAACCCAGTAAGATGATAGAGTGCTTTGCTTTGGATCTGTTCATATCTTACCCGTGTTTGTGCTCACATGAGTTACACAGTCAGTACA
AGGGTAGAGAGCTCTTGAGAATTTCTTACCTAAGGGAAAGAACTTGGGGGCTGCTTTGCTCAAAGGCTATTTTTCAGTGTGGGGCTTATCTAGA
GACATATATTTTGCTCCTTGTCCCCACTGCTGTTGCATAACTACGGGAGTTGAGGGTCACTATACACAGACACAGATGGTAGAGATGGGGGCAG
TGGTTGGGCAAGGAATAGCTGCCTTGCCACTTCGAAGTCTCCTAAATGGCATCTCTGAAGAAGAAGGATATGTCCACAGGGAGAAGGGCCCCAT
GGTGACTCTGTTGCTCCTAATGTGCCAGGCTCAGACTCTTCAAACCACAAGAAGTGGCCCAGCGAGTTCCATGTAACTGCAAATGCAAGCAGGA
AAAAACCACACTCAGATCTCAGCACAGCATCAGTCTTGGGGACAGCTGGCCTTTCCTAATGCAGCAGCATCTGGCTGCTTCTGCTGTACCTGGA
TGTCTTCAGGGCAGTTCTTCCACACAGAGGACCCCAAATAAAAGCAGAATTCCTACCATTAAGCTTTCCGTATCCAAAACTTTCTATGTGAAAG
ATACAGCATCTGTGTCTCACAAGAATCTGTTTTCTTGGGGCATCTGCTATTTGGCAGCTAACCAAAACAGAATTCCCATCTTATTTTCTTGTTC
ATTTGTGCTACATGGAAGGGACTGGTCCTGAATATTCAGGGCAACATCCAGCATTTGGTAGTAGTGGAGAGAAAGAGAAGAGAAAGCTCTGGAC
TGTGTTCCTCTGTCTGGGAATGTGACTCTCACACCTCCCAAATCCTCCCATTCACACTTTTTCTCCCTGACTTTCCAGAACCCTTTTTAGGCTT
AGCTCCTTTTATCTTACCCTACACGCTTTCTTTCTACCCACACTGGTGGGTACTTTTCAGCCATAGACATTGGAGCACATTTAAATATGCTGGA
GTCCTTAGAGAATTAAGAGGTGGGGGAGAGAGGAGGGCACAGAGCTGAGGATGGCACTAGCTCATTCACAAGGAGCCCTGAATTTCCAGGGACA
TTAAAGAACAGATGCTCATGACAGAGCATAGAGCTCTGCCCTAATTGTTTGTAATGTTGAACTATGTGTCATTCGTTCACTCATATATTCTCTC
AACATTGGAATTGAGCACCAATTATGTAACAGAGTAGGTCTTGGCAATACAAAGATGAATGAAACATACCCCTTGTCCCCAAGAAGTATGTGGA
CACGACAGGCACATGGACCAATGATGGTCATGTCAAGCGTGATGAAGTCCATGGTCCTGAGGGACACTTGGACCACGGCCTGCATGAGAGGGCC
TGGAACCCAACTGGGCGGATCTAGAAAGAATTCCTCTGGACTTGATTCCTGAACTTGAAGGTAGAGATGGAAGGAGAAGAGTTCTGGGTAGCAT
GTTCCTGGCAGAAGAAGCCCACTGCAAGAGCAAAGGCACTGAGGAAGCCTGGTGGTCACTGGAAAACTGGGATTCAAGTAGCTGAAATCGTAAG
GGTGCAGCCTAGGGAGAAGTGAGACCCGTGGTGGGGGGCAGAGCCAAATCTCTGTATCCAAACCTTTCTATATGAAAGATACAGTGTCTGTGTC
TCACAAGAATCTGTTTTCTTCGGGTGTCTGTTATTTGAGTGCTAACCAGAACAGAATTTCTAAAGATGGTCTTTGAGGCCAGGTAACAAGGCTG
AGGCTAAGAAGAACTATGGAAGGTCTGTTGGCAGGAGAGATGTATGATTAGATCTGCATTAGCAGAAGGAAAGTTCTGGCTGAAATCTGCAGAG
CAGTTTGGAGGGGACAAGACAGATGACATGGAGAACACTTAGGACGCTGTTATAATAAAATAGGCTAGAAATGATGTTCCTCAGAAGTCCCTTT
CTCCCTGCACACCTGGCCACCTCAGACTTCCTGTTCAAGGGGAAGGCAGGCTGGGGAGAGAGGTTTCTTGAAAATAACACATCTGGGCCGGGCG
CGGTGGCTCACGCCTGTAATCCCAGCACTTTGGGAGGCCGAGGCGGGCGGATCACGAGGTCAGGAGATCGAGACCATCCCGGCTAAAACGGTGA
AACCCCGTCTCTACTAAAAATACAAAAAATTAGCCGGGCGTAGTGGCGGGCGCCTGTAGTCCCAGCTACTTGGGAGGCTGAGGCAGGAGAATGG
CGTGAACCCGGGAGGCGGAGCTTGCAGTGAGCCGAGATTGCGCCACTGCACTCCAGCCTGGGCGACAGAGCGAGACTCCGTCTCAAAAAAAAAA
AAAAAAAAAAAAAAAAAAAAAAAAAAAAAGAAAATAACACATCTGTTAATTGGATCATTTCTGTGTGGTCAATGTCTGTAGGGGCTTTGTTTTG
GTCATCATAGTATAAAATCCCCATTATGTAATATTTCCTCCTGATAAAAGCTCACTGTGTGTCTATACCCTACCAGCTTCCTTATAGTATTTGC
TCATTACCAGGTTCAAGGACCTGTCTGTGTGGCTCTTCCCCAGAGCCTCTGTTGGAAAGAAGATTTGTCTTTGGTTATAAGTAGATACACAGTG
GTCAAGATGCTCTAGCACAGTGCAGCAGAATAAGATGGGCTCAGTAGGGAAAGATCATTCACCCCTAAAGTGTACACTTATTCTTTTCTGATTA
TGATAGGATTATGTTGAAATTAAAAATTGTTTCAACGAAAAAGCAACTGATTGCTTAGGCCAAAGCAGAACTTTTTTTTTTTTTCCTGAGGCAA
AGTCTCAGTCTGTCACCCATGCTGGAGTGCAGTGGCACAATCTCACAATCTCGGCTCACTGCAACCTCCATCTCCCCAGTTCTAGCGATTCTCC
TGCCTCAGACTTCCGAGTAGCTAGGATTACAGGCACCTGCCACTATGCCCAGCTAATTTTTGTATTTTTAGTAGAGACAGGGTTTTGCCACGTT
GGCCAGGCTGGTCTTGAACTCCTGACCTCAAGCGATCCACTCGCATCAGCCTTCCGAAGTGCTGGGATTACAGGTGTGAGCCACCATGCCCAGC
CAGAATTATTTTTAGTACTGTAGTTAAGAGGGCACATTCTGGAGCAAACTGCCTGCTAAGTGTTCGCTGTGGGGCATTGGGGAAGAACTTAATC
TCTTTGTGCCTTGGTTTCCACATGTAAAATGGGATAATTATATTGTCCCCCTGTACTGGTTTGCTGGGGCTGCCATAACAAAGTACCACAACTG
GGTGGCTTAAACAACAGAAATGTGTTGTCTCATAGTGCTGGAAGCCGACTCACCTCAGAAGCATGGACTCCCTTACCTCCAAGTCCAAGGCAAT
GGGGCTCTCTCCCTCTTGCCTCTTGAATTCCTGCATCTCATTTCACTGGGTAAACAGGGAGGCTCCTGTGCTCCAGCAGGAAAACCCTGGACTG
GGAAGCAGGATGTCTACTCTGATCCTGCTGCCCCAAAGCTGTGCGGCTTTAGACAAGTGGTTTGACCTTTCTGGGCTCAGTTTCCTTGATTTTC
ACTATTATTTGAAGGGCTTGGGGTCATGGAAATCATGAACTCTTCACACTGAGAGGAATCTTCATAATCCATTTTGTAGATAACGAACCTAAGG
CTGAAGAGGTCAGGGGCTTGCACCCAGTCATAGAGCTAAATGAGGTGCAAAGAACCTAGAACTCCGGCGTCTTGACTCCCACCTGGGACACATT
TTATCAGTAATAGTGAACTCTCATCTGTTGATAGTGTTAGAATTCTCACATTTACTCCCCATAACAACTTTCTTTTTTTTTTTTTAAACGGAGT
TTCTCTCTGTCACCCAGGCTGGAGTGCAGTGGCACGATCTCAGCTCACTACAACCTCTGCCTCCCGGGTTCAAGCTATTCTTCAGCCTCAGCCT
CCCTAGTAGCTGGGAATACAGGCATGCACCATGGCGCCTGGTTAATTTTTGTATTTTTAGTAAGGATGGGGTTTTGCCATGTTGGCCAGACTGG
TCTCAAACTCCTGACCTCAAGTGGTCCACCTGCCTCAACCTCCCAAAGTGCTGGGATCACAGGTGTAAGCCACCATGCCTGACCAGCACCCTTC
TGAAGTAGGTGCTTTCAGAAATCTGAAAGCATAGTGTCGGTTAGCAGGGTTGGATCTCTTCTGGTACTGTGAGGAAGAATCTGTGCCATGGCTG
TCTGCTAGCTTCTGGTGGTGTGCTGGTCATCCTTAGTGTTACTTGGCTTGCAGAAGCACCATCGATCTCTGCTTTCATCGTCGTGTGGTGCTGC
CCCTGCATGCATGTATCTGTGTCCAAATTTCCCCTTTTTATAAGAGCACCAATCATATTGAAATAGGGCTCACCCTCATAATCTCATCTCAACT
AATTATATCTGCAATAACCCTACTTCCAAAAAACACCACGTTCTGAGGTCCTGGGTGTTGGGATTTCAACGTATGAATTTGGGGTGGGAGGGCA
CAATTCAACCCAAACATTACCTAATAAAGTTTTCAACAGTAAGTAAATGAGTTAACTCCTGGAAAGCATTTAAAACAGTGTCTGACATTCAGAA
AGCTCTCAGTAAACGTTACATTATGCTGGGTATAAATAAAAAGCTCCCAGGACAGTGGACGGCATTGAGATTCCACCGGTTTGTATTAGTGTGG
CCTCGCCAGTAATTTACATCTTCTATCTTTTTTGGTTGGTCAGGGCTTAAGCCATACCAGGTAAATATTTTGAATAATACTCCTAGCTAAAAGG
TGCAAGGAGTAGATACTGTGGGTTCTGATTTTTCTTTCCCGTTCTCCAAGCTGTGTACACAGAACCATGCAAGTCGCCCCAACCAGGGAAAAAC
AACTGCCATTTCTTTTAAAGGGGAACCAATGAAGGTCTTGTCCTGATGAGCTCACAAAAGCAAACTTCTCCATGTCTGTCGCTCACTCAGTGGG
CTGAAGCAGAGCCTTCACGCTAAGCTTTGGAGACAAAGCTTCTTAGAGAATGTGCAACGTGGGCTGATCCCAGGCTGCCCAAGGAGCAGCTGAC
GAGTGTAAAAGTGTTTGCTCACCGATTAGAATCAGTCCTTTAAAAAATAAATAAGGTATGTTGATCTTTTATGTGATGATTAAACAAATCAGAT
GGCCCAAAATGAATTCCAGGTAAAGAGGAATAAAATATCAGAAGGCAAGCTGCTGAAATTAGCAAGAGCTGAACTCGAAAAGCCCGACTTTCCA
GGCACGGCTAGTTTCTTCCTAGCAAGTTCCTGACGGAGCTGGGTGGCTGCCCCAGGTGACTGCAGTAGCAGGAGATTAAGGAGACAGCACCTGC
CTGACAAAAAAGGTTGCTGACACAATAGGCCAAAGTTAAATTGAGCTGCCAAGGGAAAATGATTGGGGATATGCAGTTAACAGTTGAGGGAGAT
ACCAGAAGAATTTGTGTCCACAGAGAGATTTTGATGGGGCTGACTTTGCCGCTGACCGACTCACCTCAGAAGCATGGACTCCCTTACCTCCAAG
TCCAAGGCAATGGGGCTCTCTCCCTCTTGCCTCTTGAATTCCTGCATCTCATTTCACTGGGTAAACAGGGAGGCTCCTGTGCTCCAGCAGGAAA
ACCCTGGACTGGGAAGCAGGATGTCTACTCTGATCCTGCTGCCCCAAAGCTATGCGGCTTCAGACAAGTGGTTTGACCTTTCTGGGCTCAGTTT
CCTTGATTTTCACTATTATTTGAAGGGCTTGGGGTCATGGAAATCATGAACTCTTCACACTGAGAGGAATCTTCATAATCCATTTTGTAGATAA
GGAACCTAAGGCTGAAGAGGTCAGGGGCTTGCACCCAGCCATAGAGCTAAATGAGGGGCAAAGAACCTAGAACTCTGGCGTCTTGACTCCCACC
TGGGACACATTTTATCAGTAATAGTGAGCTCTCATCTGTTGATAGTGTTAGAATTCTCACATTTACTCCCCATAACAACCTTCTTCTTCTTTTT
TTTTTTTTTAAACGGAGTTTCTCTCTGTCACCCAGGCTGGAGTGCAGTGGCGTGATCTCGGCTCACCGCAACCTCTGCCTCCAGGGTTCAAGCT
ATTCTTCAGCCTCAGCCTCCCTAGTAGCCGGGAATACAGGCATGCACCATGGCGCCTGGTTAATTTTTGTATTTTTAGTAAAGATGGGGTTTCA
CCATGTTGGCCAGACTGGTCTCAAACTCCTGACCTCAAGTGGTCCACGTGCCTCAACCTCCCAAAGTGCTGGGATCACAGGTGTAAGCCACCAT
GCCTGACCAGCACCCTTCTGAAGTAGGCACTCTTATACCCATTTTACTGATCAGGAGACCGGCTTGCCCAGGATCCTGCAGCTACTGAATAGAG
GGGCCAGAACTTTGCTGGTTTGGTGCGCTTCCTGGTACACACCAGCTGCCTCCAATCTCCCTTTGATAATCCTAGCCACATGCCACCGCGGGTG
CTCTAGTGTAGGCTGGCACCAACCCCACCCAGCAAGAGCCAGGATCTGCTTCTTGAGGGCAGGCAGGTTTATCACACAGCGGGGTGCAGAAGAG
TAGTATTCATCTTCCTAGTGTAACCACTTATAACTACTTTTCAGTGAATCAAATAGATTTTTAAGCAAGAACATTGTAAAAATCTGCTTAATCA
AGGGTGGAGCAAAATGAGTTAGCCAGGGCCACCCTAGCTCAGATGGGGTGGTAGCTGCCTCTGCTCCACAATGAGGTATTTACACTTGCTCCCC
ACCCTCTCCTTTGCCCCATTATCTTTTAGTTAAAGGGCCAGGTGCACCAGAACACAGGTGCCTTGAGTAGCACCAGCACCTCTCAGGGTCATAG
GGAAGAGCTGTTTCCTCTGGGTAAACTGAGGCCTCAATAAGGCATAAATGCCTTCTCTGGGTGAAGAAGATATGCCCTCTAAGATCTCTCCAGG
GTGAATCTAGAATAAGTTGTAGTCTAGATGGCTTGGCAGCACTAGCCAGTGTGACAGTAAGTCTTGATGAACATCAGTCAGCGTCATGCTGAGG
GACAACCTCGAGGAGAGGGAATGTGTACTTGTTACAACAGCACACCCAATAGAAATAGCATGTAAGCTACAAATGTAACTTTATATTTTCTAGC
AAAAAAAAAATTTTTTAAAGAAACCAGTGCAATTCATTTTAATAGATCTTATTTGTTTCAATATATCCCAAATATTGTCCTTTCAATGTGTGAT
AATATAGTTATTAGTGAGATATTTTCCATTCCCTTTTTCATACAAAGTCTTTGATACTCAGTGTGTATTTCACTCTGACAGTGCACCTCAATGT
GGTGCAATAGGCGAGGGTCTATGTGGTACATCCCGTGCCCAGTGTGCACTTAAAGGCTGTAGATCCATATATGGCGACTGTCTCCTAGATAGAT
GAGCCCAGCAAGGCTCAGTGGGTGTTTTGAGGTCCATGGTCTCTCAGGTCCCGTTCCACCCCGACATTCGACGAATCAGAGAACACTAATATGG
TTGGGAACACTTTTATCTTGCTCCCCATAGTCTGAACAGAAAGAGACGCCACAGAATACAATCACGACTCTCTCCCTCCTACGGACTCCCTCCC
CACAAGCAATCAATGGGCTGGTCTCAGACAAAGACACCTAGTCAAGGCTGGTGAAACCTGCTCTATTTCCTAGTAGGCCTCCACCTGTAATTCT
CAGCAGTTTGTGTGTATAGATAGAATTCCTGCCTTCTACTAATGTGACTGTTCCTGCTCAGAGATCTAGAGTTAATTATTAGCAAATTCACAGT
GAACCAGAGCTAGGTCTTGCTGTAAGGCTCCTGTGTTTTTTAGAAGTACGTTGCCATGGGGATTTTTGTCCCCACTGGGTTCCTCCTCAGGACC
CCAGACAGATAGATACACAAACCCTGGGCCCATAATTTTAGTTAGAAAAGGCCCAAGCCTAGAACCTCAGGGAACTCATTTGGAAGCTTTTTTT
TTTTAGGAAACTTACCTTTCCTATTTGACATCTCAATCAAACAGGTGTTTGGGGAGAATAAAAGCATACTACCTTAGAAGGGACTGGGGTCATC
TGGCCAGACTTGTGGAATAAAGAGAATTTCATTCCTTGTATAAAATAACTCTAAATAATGGTAAGCTCTTAGGAGACTTGATCTACTGGCTACA
TATACCCACTATGAAATTAACACATTCTGGAATTTAGATGGAAGAATATGGAAATTGGTCCTTCTCAGTGTATGGACGAAGCATCAGAAATGAA
GAGAAGGGCCAAGCCTAGAGAGCAAAACTAAAGACTCACTGGTCTGTGGGCTTCCTGCCCAAGGAGAGCTGAAAGCTAAAGGATGCTTCACAAT
GTCCTACAAGTTGTTTTTCTAAAGTCCTTCCAGCGGTTGAAACTGTGTTTATGCAAAACAAAACTTTCCTTTGTTGCTATAATCTAGAGGTGGA
CTTTGGCTTAAGTCATGCCAAAATATTATCATTCTAAACCAGTAACACCTATATTTGTTGAGCATTTACTATATGTGTGGCACTATGCTAAACT
TAACTACCCCTTTTCCTCATGTGTAACAGTCAGGTGACTTCACAGCTGCCTTGTGGATACTTGTGTTATTTGAAAGATTGCATAAACACATGTA
AAATGCTTAGAAGTCTACCACCTCATCTTACCTACAGCTTTGGTGAGTCCCTGCTTTGGCCAAGTAGAATCATTCCCTGTGAGGAGGGGAAGAG
GATAAGAAACAGGCAGAGTTGACCTTACAGGTAGACACAACAGGCGAGGGTCCATGTGGTGCAATGGATCCCATGCACATGCCTGATTCAGTCA
TTCTAGTTGAGTAGGATTGTCTGGTTGGTTGTTATTATTTTGTTTTTTGTTTGTTTCTTTCTTTTGTTTTATAAGATGGAGTCTCACTCTGTCG
CCTAGGCTGGAGTGCAGTGGCTCAATCTCGGCTCACTGCTGCAACCTCTTCCTCCCGGGATCAAGCAATTCTCCTGCCTCAGCCTACCGAGTAG
CTGGGGCTACAGGGGCATGCCACCACACCTGGCTAATGTTTTGTATTTTAGTAGAAATGGGGTTTCACCGTGTTGCCCAGGCTGGTCTCAAACT
CCTGAGCTCAGGCAATCCTCCCACCTCAGCCTCCCAGAGTGCTAGGATTACAGACGTGAGCCACCGTGCCTGGCCAGTTTTTATCTTGAAAGCT
TAAGGGTCTCCCACTTAAATTCAAATACTCTTCTGAGGATAACCTTTTAATCAGCCAACTATCATGAATCTCAATTATCAGGAATTCCAGTTAT
TCCAATTATCAGGAATTCTTTATATCATCCCAGGAATCTAAAAGAGCAAGGTCTTATAGGTAAGGGCAAACTTTCTGATGCCTTGGAATTCAGA
CCCTAAGTAGAGATGGTCATTTGACATCTAGTCTGATCGACTGTAGAGTTACTTCTATACAAATAAATTCAAGTTATCACAGGGTTTCTTTCAG
TTGGGGACAATACTTTGAAAACTTTTAGGCTCTGAATGAATAACTTGGCAAAAGAAAATGTTAGGGTCCTTCAAATACCATACTTGTTGTGGTT
TTCTCACAGATACACCAGTCTATAACCCTGAGGTTTCAACACTCCACTCAGTTGTGGGCTTTTTATCTAAATCAAAACTATCACATGCCCACAT
GTGCATCTGCAGCCAACCACAAATGATCTAACAGGACCAGATTTCACTGGAGCTCTGTGACCCTTCTCCCAGTGTGAACTGAAGGGAAAAAAAC
CTGGAGCCTAAGGGACTCCCTTTTCCGTGTCCTGAAGACCACTGGCTTTGGTTAAGCCAATATGATGGGCTCATTGAGAGAGAGACTCAGGACC
TCGGATCATGCCACACTCTCTGGCCCCAGAAGGCATACCACAGGCTGCCAGCTTTGTTGGAGAACCTGAGACTTAAGGCCAGGATATATCAAGA
GCCCTGTACCCAGTTCTGTGATGTCTTAATACCTTTGACAAGAGACATTGGCAACTCACTGGGTGTCCATTGGGGGGCGACAAGGAAACAGACT
GATGAAACCAGAGAATGTGACAACTAATTACATGTTGTTTCCTGTAGAAGAGAAATTCTCAGAGGAACGTGGTGACTGTCTGTTCTTTAATGGA
CTGTCATATGAAAGCAGGAGAAGGAGATTCTTCCTTACTCCAAATGACAAAACTAGGAGCAGTGAAATGAGCTTATAAGGTGGCCGGGTTTTTC
TAGCAATTGGAGCTCTCTGCAGTGAGTGGACACTTGGCAGGGTAGTGCCTACTCCGCATTTGATGTGTTCACACCACGGCTAGAAGTAGGTGTC
GCAACTTCCAGTTGAAGAGGGAATTCTTTTCCTTGGGTGGGAGAGGGGCTAGAGAACGTCTCAGGTTGTTGCTAACTATGAGCATGGTTTTATA
ATTGTTTAAAAGTGGGTAAACAAGAAGAGTGTGTTTATGGTGTCTGTATTCCACAAACTATTATTTCCCACATTAACTGTTTCATATATGGAAA
ATCTGAGAAACAGGAATGCTAACAAGGTTGCAGTATAATTTAAAAATACTTCTGAGAGATCATTGTATCCCAAAGGCAGTGCCCAGTGAGGTTA
AGAGCGTGGTTTGTGGAATCTGAGCCCACAGGGGTTTAAATCCTGCCTCTGCCCTTCTTAGATTCTGTGAACTTGGACAGATTCCCTCACTTAT
TTAAGACTTCACTTTTCAGCCCTTGAGATCCCACCTGCTCAATTATCTAAATGAAATCTATCACTGTGGAAAGAGCAAAGGCCTCCTGCTGTTG
AGCAAAATGTTTTCCAGTCCTTTGTTTCTGTGTGAAATAAAGCAGCTCCATCCCAGTGCTAAATGATCAACTGCATATGACAGAATGAGCTTAT
TTCATGCTCCCAGTAAATACAAGACCATAGACTACTCTGGATGGTAGTTTAAGTTTAAGATGAAATTGCTTCTTCTGATTGCCTTCTTCTTTAT
TGCTTCTTTCTCTGTTGTTCCTTCCTTCTCTTGTTTATAACACTAACAGTCAAATCTATCTGTGTGGAAATCTATCTACTATATAGAACGCTAC
ATCACAATTTACAGAGGGGAAAATATTTTTGTCGGCAGTAACATTTCACTGGAAGAGGCACTATTTGGCCACCTAACTCTGGGATTTAGGCTCA
GGTAGAATCAGTGCCTCAGTTTCTTTAGGTCAGCCATTATGAGTTGTTCCTTATTTGACCTAGCACATATATAGATGGTGGTTAAATTTAGCAA
AATTCTCTTCACAAAGGTTTCATTTGTCTGGAAGACTCTAAGTACTGATCTGGTCAGAGTACCATCAAAGCCCCTTGTTTGTCATGAGACCGTG
GACCAACATGTTAGTCTTTCTCATGTAAAAAAAAAAATCATCAACAAATGCAGTGGGAGTTGGTAGTCATGAGTTTGAGGTAGTACAATATCCT
TAAAGTACACACTCAATCATGTGTGATAATTGGCCAACTCTACCTAACAACAGTTGAAGACCTCCAAACAGCTATCAGGACTACTGGAATCATG
TCTCCAGAAGAACCTGGAGAAACATTCACATTTGTGTGGACCAAGGAGGAAAGCAACCCAGACGACTTCTACTTAGAAGCACAGGCACATAGAG
CCTGTGTTATTGGCACAGGTATTTCTCTCCTAAGAGCCTTTTCTGCTTAAGAAGGCTTGAGGATCTATTCCCATTAATCATAAAAAAGGGATTT
GCTGGGCTAAATTATTTTAGCCTCCTCTTTATGCCTTACTTCACAAAGAATTGCAGTTAATCTGTAAATGAGTTCTGATGTTATGAAAATTGGA
TATGGCAAATTGTGTTATTAAGTCTATTAAGGGATGAAGAAGGACTCTTCTAAAAGAATAACGTAGTCTGAGTGGCAGCTCTCACTTAATAGAC
ATCTACCTAGAAATGTTTCTGAACTTAATGGACACATGATATAATTCAAAATTAATATAAGCACACAGCACCTCTCCCCATAACCTTATTGAAA
TTAAGGGTTCCAAATCATTCTTCTCTCCCTTTTACTTCACCTGCAACATGTGGCTCACCCTTCTTTAGACTAAGAGCTAAACTCTAATGCCTAG
TTAATGTAAAACAATGCCAGGATTATTAAGCATGATTCTGATAAGTTTTAACCCCTAATGAGATTATCCTTGGTTAAATAAATTGAATGGCTAG
TGGAATTCCAATGTAAAGTCAGCAGAGGTTAGCTGGTTGCTAGATATTTAACTATACATACCAGGAAGCTATGAGGACAAATTTCCATTTGCAC
AGAGCCTTGAGAATTGGCCATTTTAGGACTAAATGTGCTGCTCATTACTTTTACTCAAGGACATACTAATGACCAATATATGTTTAAATACCTA
CTATGTACAACTGCTTTCAAAAACACTTTCAATCTTGAATTGAGAAGGTAGGAGATCCATACCTGTATTAGGCCATCCTCACAGTGCTATAAAG
AAATATCTGAGACTGGGTAATTTATTAAAAAAAGAGGTGGATGAAGCTGGAAACCATCATTCTGAGCAAACTATCACAAGGACAGAACACCGCA
TGTTCTCACTCATAGGTGCGAATTGAACAATGAGAACACTTGGACACAGGGTGGGGAACCTCACACACCAGGGCCTGTCATGGGGTGGGGAGAG
TGGGGAGGGATAGCATTAGGAGATATACCTAATGTAAATGACAAGTTAATGGGTGCAGCACACCAACATGGCACATGTATACACGTGTAACAAA
CCTGCACGTTGTGCAGATGTACCCTAGAATTTAAAGTATAATAAAAAAAAAAGTGGGGAAAAGAAAAGAAAAGAGGTTTAATTGGCTCACGGTT
CTGCAGGCTATACAGGAAGCATGATACTGGCATCTGTTCAGCTTCTGTGGAGGCCTCGGGAAACTTACAATCATGGCAGAAGGCAAAGGGGCAA
CAGGTATGTCACATGGCCAAAGCAGGAGTGAGAGAGAGAACAAGGTGCCACACACTTTTAAACAACTACATCTCATGAGAAGTCACTCACTATC
ACGAAGAGAGCACCAAGGGAATGGTGCTAAACCAAGGTCCCCAACCCCCAGGCCACGGATCAGTATTGGTCCATGGCCTGTTAGAAACCAGGCC
ACACAGCAGGAGGTGAGTGGCGGGCAAGCAAGCAAAGTTTCATTTGTATTTACAGCTGCTCCCCATCGCTCATTACTGCCTGAGCTCAGCCTCC
TGTCAGATCAGCAGCAGCATTAGATTCTCATAGGAGTGCAAACGCTATTGTGAACTGCACATGTGAGGGATCTAGGTTGCACACTCCTTATGAG
ACTATAATGCGTGGTGATCGGTCATTGTCTCCCGTCACTCCCAAATGGGACCGCCTAGTTGCAGGAAAGCAAGCTCAGGACTCCCACTGATTTT
ACATTATGGTGAGTTGTATAATTATTTCATTAAATATTACAAGGTAATAATAATAGAAATAAAGTGCACAATAAATGTAATGTGCTTGAATCAT
CCCAAAACCACCCCCTCTCTGCCCCGTCCCAGTCCATGGAAAAATTGTCTTCCACAAAACCAATCCCTGGTGCCAAAAACTTTGGGAACTGTTG
TGCTAAACCACTCATAAGAAATCCAAACCCATGATCCAGTCACCTCCCACCAGGCCCCACCTCCAACACTGGGGATTATAATCCAACATGAAAT
TTGGGTGGGGACACACATCCAAACTATATCAATACCTTCATAAAAGCAATTAAATAACTGTACAGTGTGATAGGCAATAAATGTCAAATGTCAT
ATGTATGAGGAACATAGAGAGGTGCTGTTATCAAGTCACTGTGTGACCCACTGAGCACTCGCAATGAGGCACAGGTAGTAATGTACCTGCTCAC
TTGCTTATTGTTGTCCCACCACGCTGAATTGGGAGCTTCATGAGAGCAGGGCCCATGTCGCATCTATTACCACAACTGCCTTATTCGCGTGGCC
TGTAGCTAGCAATCAGTAAAAAATTACAGCATGGATGAAGCTACAGAAAGAAAGTAAATACTGACAGGTGGGCAAAATTTTTAGATATGGATAT
TTAATGATGGGATGAACAGATGAATCAAGACAGGAAACAAGGCTTCGCGTGGCAAATATATCCTTTCATTTGCCCAGCAAACATTTGCAGATCC
CTCACTATGTGCTAGACACTGTGGGCACATTTCTGTGTGGATTGGTGTTTTTCTCTCTCAAAACTGGAGAGATTTTGAGATCAGCCTGTATGTA
GTGAGATGAAGAGGTATGTGGTTGAGATCAGGAGTAGTTGGGATTCTGTGAGGGATAGGGCTCCTTAGGTGTAGAGAGCCTTGAGACCTTTGTT
GTGAGGATAGTGAAAATCAACAAAAGAATTTATGTTAAGGAAGGTTAAAAAGTCACTCTAAAGGTCTCAGAAGTGCCTATGACATCATGAGGAG
ATGTCCATTCCTGAAAACTTTAGCCAGGTTACAAACACTTTAAGAAATCCTATCTCAAAACTTCAAGAAAAGTGAGATCTGTATTTACACAGAT
AGAATTTCCAGGACTGGATTATATTTGCACTTTTTTGTTAAATTTTTACTTAACTAGTATATTAATATTGATAGTACTTTGTGACTTTTTTCAT
AAATATAACTTTTAAAAAATATGGTATTTCTAAAAAGGTTTGGTGCATTGTTAAAATGTGGTTTTGATTTATTCACAGACAGAAATCTTCTCTA
ACAGACTATACTTCCATTTCATAATTCAGTGAGGTAATGGGACTCAGTTATACTAAATTTATCATAGAGCAAACTTTGCTAGTTTTCATCTGTT
CATTACATTTTATGTAATGGCAGACAGTGTAATAACTAATACCGTTAACCCAACCATCTCTTCCCTATAATGTTTCTATAGGAACATACAGTGT
ACATTTTGCAGTTCTCTACACGCAGACAAATTCTACCAGTGTGTATTCTGATTACAAGCTAGTTGCTTCATAGGCCATATGTCTATCTTCAGTC
CCTATGAATCATGTAAATTACAGAGCAAACTGCCGATACTCAGTATATGTATATGACACATAAGAGTCAAGTATACTCAGCTCCTAAGAGCAAG
TGAGAAGACAAAGAAGATCTACTGTGTGTAGAAATACTTCTTAAATCATCTTTCCTGAAGGCCTAGTTTGCAACTGAGTCGCAAAAATTTGGTT
TCAAAAAGTTAAGCTTGCAATAATAAAAAGATAACCCAGTTTAACAATGGGCAAAGTGATCTGAACAGACATTTCTCAAAAAATGATATAGAAA
TGGCCAGTAAGCACAAGAAAAGATACTCATCATCATTACTAATTAGGGAAATGCAAATCAGAACGACAATGAGTCACCTCTTCACAGTCACTAG
GATGGCTATGATTAAAAAAAAAAAAGACAGGCAATAACCAGTGTTGGCAAGGATGTGGACAATTTAGAACCCTCATACACTATTGGTAGACATG
ATATATCCGCCAAACTGAAAACAGATACTCAAGCAAATACTTGTACATGAATGTTTATAGCAGCCCTATTCACACTGTCCACATACTTGAAAAC
AATTCAAATATCCATCAATGGATAAATAGATAAACAAAACGTGGTAATTTGTACAATCGAGGATTCAGCCATAGAAAAGAGTGAAGTACTGATA
CATACTAGAACATAGATGAACCTCAAAAATGTTATGGTAAGTAAAGGAAACCAGTCAAAAAAGACCACATACTTTATGATTCCATGTGTATGAT
ATGTCCAGAATAGGGAATTCCATACAGACAAAAAGTAGATGAGTGTTTGCCAGGGGCTGGGGAGAGGTGGGGATGGGGAATGACTGGCTGATGG
TTAGTTTCCTTAGGTGGTGAAAAAAATGTTTTAAAATTGATTGTAGTAATGGATCCACAGCCTTGTGAATATACCAAAAACCACTGAACTGTGT
AGGGGTGGGTTGCCCCTCCACACCTGTGGGTGTTTCTCGTAAGGTGGAACGAGAGACTTAGGAAAGAAAAAGACACAGAGACAAAGTATAGAGA
AAGAAATAAGGGGACCCGGGGAACCAGCGTTCAGCATATGGAGGATCCCGCCAGCCTCTGAGTTCCCTTAGTATTTATTGATCATTCGTGGGTG
TTTCTCGAAGAGGGGGAGGTGTCAGGGTCACAAGACAATTGTGGGGAGAGGGTCAGCAGACAAACACGTGAACAAAGGTCTTTGCATCATAGAC
ACTGTAAAGGATTAAGTGCTGTGCTTTTAGATATGCATACACATAAACATCTCAATGCTTTACAAAGCAGTATTGCTGCCCGCAGGTCCCACCT
CCAGCCCTAAGGCAGTTTTTCCCTATCTCAGTAGATGGAGCATACAATCGGGTTTTATACCAAGACATTCCATTGCCCAGGGACAGGCAGGAGA
CAGATGCCTTCCTCTTGTCTCAACTGCAAGAGGCATGCCTTCCTCTTATACTAATCCTCCTCAGCACAGACCCTTTACGGGTGTCGGGCTGGGG
GACGGTCAGGTCTTTCCCTTCCCACGAGGCCGTATTTCAGACTATCACATGGGGAGAAACCTTGGACAATACCTGGCTTTCCTAGGCAGAGGTC
CCTGCGGCCTTCCGCAGTTTTTGTGTCCCTGGGTACTTGAGATTAGGGAGTGGTGATGACTCTTAAGGAGCGTGCTGCCTTCAAGCATCTGTTT
AACAAAGCACATCTTGCACCGCCCTTAATCCATTTAACTCTGAGTTTGACACAGCACATGTTTCAGAGAGCACGGGGTTGGGGGTAAGGTTATA
GATTAACAGAATCTCAAGGCAGAAGAATTTTTCTTAGTACAGAACAAAATGGAGTCTCCTATGTCTACTTCTTTCTACACAGACACAGTAACAA
TCTGATCTCTCTTGCTTTTCCCCACAGAACTGTACACTGTAAAAGGGTGAGTTTGTGGTATGTAAATTTATCTGAATTTTTAAAATAAAAAAGA
AAGTTAAAAAATGTTAAACTTGGAATTAATGATGGTTTTTTTGAAGCAGATTAGTAACTACTGAAATTAGTCCTTGATTTTTGGAAAAGCCTTT
AAAAAATGGTAAGTGGGAGAAGTTATAAACAAAGAGATGAATGAGTCACAGAAAATTGGGAGACAAATAGATGATAACAAGGGACAGTACAGAA
AGACTCAACTGAGTTTTATCAAATCACTCAGGCAGATAAACATGGGGGCTGCATGTGCATCCGTATGTGTGTATATGTTTATTTACATCTAATA
TACACACATGATATTACATGAAATATTATAACATATAATAAATATATATTCATACTGGAAAAATTCTCAAAGAATATACCACCAAATGTTAACA
ATAGCTGTCCCTGTTTAGTGGGATTTCAGGTGACTTAGATCCTTTCCCCCTTTGGCCTCTCAGTATCTTATAACTTTGTGTGCTAAGATCATTG
ATAGTTGAACAATAAGAAGAAGAAAGTGAGTGTGGAAGGATGTTGCTAGAGAAAGTGTATTCTTGATTATATGGAGGAAGTGGGGGCAGTTTGT
GAAGTCTTCCTTACGAATAGCATCCTATTTGAGAAGACCAGTCTTCACACTGGAAAAAATGCATAGAGCAAGGTGGGCATCACTTGCAGAACAG
TTTGGCACAGGACTACCTCTGAAATATTAAGGCTTAGGAGACTTGTTCTTAGAAACTCTTCAGGCAAAGTTTACAAAAGGCCTTTTCCTCCTTG
CTTAGCAGATTCTCCCAAGGCATCCACATCTACACTCTCCGTAAGTCACTATATACTGGACCTAGCACACATCTGTCTACCCATCATTGTATTA
GTCTGTCCTCACGCTGCTTATAAAGACATACCTGAGACTGGGCAATTTACAAAAGAAACAGGTGTAATGGACTTACAGTTCCATATGGCTGGGG
AAGCCTCACAATCGTGGTGGAAGACAAGGAGGAGCAAGTTGTGTCTTACATGAATGGCAGCAGGCAAAGAGAGAGAGCTTGTGCAGGGAAACTC
CTGCTTTCAAAACTATCAGATCTTGTGAGAATTATTCACTATTATGAGAACAGCATGGGAAATACCCGCCCCCATTATTTAATTATTTCCCACC
AGGTCCCTCCCACAACACGTGCGCATTATGGGGGCTACAAGATGAGATTTGGGTGGGGACACAGAGCCAAACCATATCAACTATCTATCTATCT
ATCCATCCATCTGTCCATCCATTTATCTTTAATTGGGATACGTCTACAAGACAACTGATACCTAAAAGTATGGGAATTTCATTTGCTAAGTCAC
AAAATAACTTTTAAACCATCCTTATATTATACCTAACATGATCTGAGCACATAATATTGTCTAAGTCAGACCCATTCTTGCCTCTCTGAGCTTA
CACTCTTGTCAAGTGTGAGCCAGAAGGAGTTGGTCCAAGTAGAGCAAGTCTCACAGGCTTTGTGGTACTCAGGGTGGGGGTCTTACCCAACATT
TCCTCCCAGAAGCTGAAAGGCAGAGACAAGAAATTCATCCTGAAAGAGTTCACAAGGGTAAAGTCAAGGTGGCTGGTCCCCAGACAGGTCATAG
GAATAAGGTTCTGGTCATACAACAGGAGGCAAGGAGCCCAAGTAGAGGGACTTGGGTGTGGGGCAAGTGGGAGGGTTTCATTGAAAAACTAGGT
AGAAGACAGGGAGTAATGGTGGATAAGGCAAAGGACAGCATCTAATTCAGGCCTGTCATCAAGCTAGAGAATGTTCACAGCAGTGTTGTTTGAA
AGAGAAAGGCATGGAAAACACTTTGTTGTCTATCAGCAGGTGTGAATAATTTAATTGTGGTGTTTTCAAATAGTGAGATTTCCAGCAGAGGAAA
TTAATAAACCAGAGCTGCATGTGTCGATGTAGAAAAAAACCCAAAGTGTCCAGACATGGTGGCTCACGCCTGTAATCCTAGCACTTTGGGAGGC
CGAGGCGGGTGGATTGCCTGAGCTCAGGAGTTCGAGACCAGCCTGGGCAACATGGTGAAACTCCGTCTCCACTAAAATACAAAAAATTAGCCGG
GCGTGGCGGCCTACGCCTGTAGTCCCAGCTACTTGGGAGGCTGAGGCAGGAGAATTGCTAGAACCTGGGAGGCAGAGGTTGCAGTGAGCCGAGA
TCGCGCCACTGCATTCTAGCTTGGGTGACAGAGTGAGACTCCGTCTCTTAGCAAAAAAAAAAAAAAACCGAACACCCAGTGTTCTCACTCATAG
GTGGGAATTGAACAATGAGAACACTTGGACACAGGAAGGGGAACATCACACACCGGGGCCTGTTGTGGGGTGGGGGGAAGGAGGAGGGATAGCA
TTAGGAGATATACCTAATGTAAATGATGAGTTAATGGATGCAGCACACCAACATGGCACATTCATACATATGTAATAAACATGCACGTTGTGCA
CACGTACCCTAGAACTTAAAGTATAATAAAAAAAGTAAAAAGAAAAGAAAAGAAACAACAACAAAAAAAAACTACCCTTTAAAGGAATGCATAT
AAACAATAATTTATTTCTTTCACATTCATTTCACAGTTTGCTTACTGAAGCATCAGTGTGTCTTTCAATTGGATGTGATGTGTTTTGAAGACAT
AAGAGGCAGGAAAGTTTTGGTTGCCAAAAATAAAGAGAAACAGGGAAGAATTTAGCTTCTGAACTGGATGGTCACTCTTGCCAGAGGCCTTGGA
GGAGGGCAAGGTTTTGCATTTTGTTTGCCAATTTTTTCCTTAAATAAAGTGCCCTGGACTTCAGGTCAGTCTTGCCCCTTGAGCTGCTAAATGA
TTCTGCCAGATTCTTTCAGCTGAGCTGTGCGGGGTCCTGAAGACCTGCTTTGACTTCCTCTGCACCAAACTGACTCACTCCCCAGGGAACAAGA
TCAAGGCTGAGGAAGAGAAAAAGCAGGGCATGAATGGACCCTATACCCAAAATAGTAATTCCATTTTCTCCACCTGCCATTCTACCATCACAGT
GAAGCTTTTCTAAATTGTAAGAGGCTCTTGTAAAAATGAAGCCACTCCTGGGAGATGTTGAATTTTATCAGCCAACATGTATGTGGGTGTCCAT
GGAAAACAAGACCTTGGCAAATTATACCACAAATGGGCTGGGGCAAGTGGGAGGAGAGTGATAGGAGAACGTTTGAATCAGGACAAAACGAGTG
AACAATGTGGAGGCAAAGGGTGGGAACAAGATGTGCTTTGCCAACCAGACAAGTTTTTCAAGGCTAATTTTTCTAGAGACCATCTTAAGAACAA
TTGCAACACTAGACAAAAAGCACACAAACCAAAAAAAGAAAAAAATCTAAACCCAACAGCCTAAGTATCAGAAACCATCTTCTTATCCATATGG
TTAAAAAGTCAAAATGAATTTATTACATTTTCACAGAACCATTTCCTGGGCCGGAACTCATTTCTCCTTAAATAAATACTATAAAATTTTCCTC
AGAACAAATCAAGTCCCCTCTATAACCCCAGAAGACAAAGTACATCGGCAGTAAAAACAGACACAAAGTCTCCACTGTTGTCAGAGGTAGCATG
GCCTGGTGGGAGGACAGGGAGCTCATGCTGCCGCATCAGTCGGGAGGAGGGATTTGTCTTCTCCACAGGGCAGGATCCCTACAGGAAACAGGAC
AAACTGAGAAGGGCTTGACAATGGAACTCTTTACAAAGGTGTCAGCAGAGTATTCAAAACCACAGGAGAATTGAGTGTCAGGGCTAGCAACTGT
ACGGTTCCAGAGCCAACCCATGTCGGAAGGGTCAGAGGGTGGGACAGTCACCACAACCCCAGAAGGGAGAGTTCTGTAGAAAGGGCCATCTGGA
GGGAAGCAGTGACCTTCAGTCAGCCCAAGGTGACAGCCAGGCAGCAAGGAGATGAGTACCCTCAGCCTCACTCCTTCCCTTCCTCCAACCTCCT
ACCAAGCCTCCCTGTGGGTCAAGCTCATCCTGGGAGTCAGAAGGCAAGAGAGCCCCTAGATGCAGTCCACATAGTTCAGAGCAGGTTGCAGGTC
AGGAAGGGTGAAGACAGGAGAGGCAAAGGGAAGACATTGGGCAGGTCTTCCAGCTCACGGCCCTGTCATGCCTGCAGTCACTGTAGTACAGACT
GAATGTTCACTGTGTCCTGCTTTCAGCATTTAAGATGCCTCTGAGATAAAGAGCCTGCCTTGAGTCTCACGGGACATGCTCACATTTTCTGAAA
CCATGTAGCTTGCACTGTTAACCTCTTCCTCGGTTTAGAGGTTTTAGGGGCTCGTTTCACAAAGCTGCCCCAGCCCCTCATGTTAAACAACCTG
AGGGCTGTGACTGGGCAAAAAATAAATAAATACATTTGCATTCAACATTGACAAAATACACACTTAAGTGAGACTTTTTTGCATCACAAAAAAA
TAAGTTTTTACTTTTTTCAAAAAGTAAAGGTTCTTTAAAAGATATCATCTAGAGTAGAATTAAGGGCTATTATTAGAAAATCACAGTGGTCCCT
AGGCAAGACATTTTGAGGGATGAACTTAAGCAGGCCCCAGACTCACGTCTTCCATGTCCTACAACCCAGGCATACCCCATCATCCCATACCTTC
AATTTATCCAAATACTTCCTGTTTCAGTTGGCAAATTCCACTGCCCCAAGCTTCCAAATGTCTTCCACTGCTGGCTTAGCCTTCTTCCTAAATC
CCCTCCCCATAGTGTCCTGGACCCCATTAAAATGCCAGCAATGAAAGCATCTCCTTTCCCCTGTCTAGCACAGCCAGGTACATCTGTTCTGAGT
GGTCAGTGCCCAAAACAGCCCAGTTGCTAAACAGTTTGACCATCACCTATGCTCACACAGGAGTTACTGAGATGATGGTCCTCAACTGCAAATA
AAAAAACACAGGCCACTCTGTAGGAGCCATTAGCTCACAACTTGCTGGGCAGGCTCAAGTTTTAGGACTCAGCATGCTGAATAACACCTCTATC
CAATGGTAAATTTCTCTGGAAATGAATCCAAAAACCTCTCAGATGACAGATCCTGGGACTTTCAAGTTGTTACATAGCAGCAAGGGTTGCTGTC
ATGATTTGTTTACTCTTTGATTCCCTGCTGTGGCTCATTTGAGATCTTGGAGTGTTTGCTGCAGTAATTATGGCAGGATTTTTCCAGAGTGGTT
TTAGCATCAGTGAAAGAATTAGTGTTCATTACTCCTTAATTCTATCACTCAACAAATTATAGGGGTAAAATTAGATCCTATTAAAAAAAAAAAA
AAACAGGGAAAAACAGCAAACAGAAACAAGCCGGAAAAGAGGAAACTAAATTCACCCTCTAATACTGATAAGCAATTCTTCCCTACCACTAAAT
TTTAAACCTTCATCAGAGACAGTACTTTGTCCGTTTGTTCAATACTTTATTCTTAGTGCCTATTACAGTGCCTGGTACATAGTTGGCCCTCAAT
ATATGTTTATTTAATGAACTGATTAAATAAACTAGACTCAGGCTCTGACCCTTGTGCTAAATCTGTGCTCACAATAGATGTTTTAGGGCATATT
AAATTTTGAAGTCAGTTCATCTCTATGGAATTTATAGGCAAAGGGGCTTTCTTTTTTTAGAGGTAGGTTCTTGTTCTGTCACCCAGACTAGAGT
GCATTTTGCATTTTGAGTGCAGTGGTGTCTGCCAGTCAGACTAGAGTGCAGTGGTGTGATCATAGCTGACTGCAGCCTCAAACTTCTCGGCTCA
AACAATCCTCCCACCTCAGCTTCCTCACTAGCAGACTACAGGCACATGCCATTGCACCTGGCTAATTTTTTAATTTTATTTTTTTATTTTTGTA
GAGACAGAGGTTTGCCACATTGCCCAGGCTGGTCTAAAACTCCTGGGCTCAAGCTATCCTCCTGCCTTTATTTCTATTTTGAAGCAAACAAAAT
GATGCAAATGAATAAAGAGTCCTAGGAAGAAGTGTCCTCTGCTCTTGACTGTCCACTAACTCCAGACTTCATAGTGGCCCCCTTTGCTTAAGGC
ATATGATAAACTTCTAATGATGCTTGAATTATATTTCCACCAGCAGGATCAGTTATTTTCCTCAGACTCAAATTTTGCAATCAAAATGCACAGT
GTACACACGGCTGCTTATATTGCAGCACATAGCATCTATTTTACCATTTCAATTTGTGTGATTTCTTACATGATGGATTGTGGGATGACAATAA
TGTGTTCTCAGTGAAGCCTCTTGACTAAACCAAAATTTGCATTTTTATAAACCTCCATGAAACAGCTTTACTTAGTAAGATTCTATAATGCTAT
ACTAAAGAAAATTACGTGGTAGCTTCTAAAAGAAATACTCCCTCCATCACAGAGACCGAGCCTCATATGAAAACAGTAAGCAGCACACAGGAAA
CTCATGCTGTAGATGAATTTGGAGAGCCACCTCAGGTTATTTGCATTATAGAATGCTAAACGCAGACTATGCAAGAAACCAGCCACCTAGAGAG
ATCTATATAGAAGTAGAGAAACTTCCATACCACCAGCAATTTTGGCTTTCCAATAAATATTGAGTGATGTCCAAAAGTCGTATAATTCAAAAAG
TCTAGGGTAAGGAACAAGATAAAAGCTAACCCTTGTGGGTGTTGGTAAATCAATAATTAATTGGAGATCTTCCATGATATTTCCTCATAAGAAT
CATTGCTAAAATTGTTGTAATGACTACACAGACTCCCAAGATTGCAATAGTCAAACAACACAAACTGTTGAAGTCATGAAGGGATATGCAACAT
GGCTCCTTGGTTAACCTTTTGGTAGGGATCCAGTTTCCACCATCATGAAGTCCTGTTTTCAATATTAATTTACATCATTAGAATGCCTGCTGTT
CCATCAGCATTTTCTTGAGATTTCCTGGGCCATTCTCAGAAACCAGAGTTAGAATGCAGAAATTAATCATATTTCATTTTGACCTAACTATTAT
ACCCAGATATTTATAAGCCTGAAGTATATTTTTGCCTTACTGATAAACCAATAAGAAAGAACTTGAATATCTCATAAAATAAGGTCTTTCCGCA
AGCCACACATATCTAGATATGGATAATAGAGCTAAATAATCAGGTGTCGAGTCCAATACACTGTCAATCTATGTTTGACAAGCATCCAGTTCCT
TGCCTTGAGTCATTCAGTTCTTCGTTGTTCTTGCTGGAATGAAAGCCTATACAAAACCTAGCATTAATGAAGTTAATATGAAACTAATTAGCTG
CAATGCTACTGCGGGAAAACGGCCAATCATCATTTAATGAATGATTCTCAGTTAAGCACTTCAGAAAAGTATTTCCTCTGCCCCATAATCACCC
AGCTTCATGAAGATCCTGCTTTCCAGGTTTGAGGTGTGGGATCCAGATTCACTTATAACCTAATCAGGAAATGAACAGGATGATCTTGTACTGA
AGTCCTATATTAGTAATATATCAAATGAATAAAGAAAAATGTAAATAAAGTTAGAACTTGTAGTATCGTTTTATCAACTCGCAGTATGTGAAAT
CCATGATGTACCTGCACCATAAATAAAACAAGATCTTCAAACTTCCCAGGGCTATTTAATTCTTCCATGATTTTTGTTCACAAAGAACAATCAT
TGCTCAGCCTATTCATTGCTCCTTTTGGAATGAAATACTTTTATTACTATTCACTTCAAAATATTTTTAAATTTCTGTTGTGATTGCTCCTTAG
ACCCACGGGTTTATTAAAAGTATATTGCTTATTTTTAAATATTTAGGGATATTTTAGTTACTTGTCTGATATTGATTCCTAATTCAGTTTCTCT
GTGGTCAGAATGCATGTTCTGTATGACTTCAACCCTTTGAAATTTGCTGAAACAATGTTATGATTCAGGATATGGCCTTGGTGAATATTCCATG
AGTACTTAAAAGGAACATACATTCTACAGTTGTTGGCTGTAGTGTTTTATTATATAAACGTCAATTAAGTCATTTCTTAATCATGTTGAGAAAT
CATCTATGCCCTTACTAATTTTTTATTTTCATGTTTAATCAGATATGAGAATGGTGCGTTAACATCTTCTATGATTGTGTATTTGTCTATTTCT
GCTTTTAGTCTTGGAAGTTTTTGCTTTGTATATTTTGGGTCTATGTAATTAGATGAATGTAGATCTTTATTTTCCTGATGAATTGACCCTTTTA
TCATTATGACATGTCACTTTTTATATTTAATAGTATTTCTTGCATTAAGTTTTAATTTATCTGATATTATGTATAACTCTACCAACTTTCTTCA
GATTAGTGTTATGATGGTCTTTTTTTCCCATATCTTTATTTTCAACCTTCTTGGTCCTTATTAGAGTATTTAATCCATTTACATTTAGTGTAAT
TACTGGATTATTTGAATTTAAACCTACCATCTTAGTATTTCTTTTTCTGTTGTCTCACCTATTCTTTGTTTCTATTTTAATCCCTGCTTGCCTT
TATAAAAATTTTAAACAATGACAATTTTTAAAATATTTTCATTTATCTTCTTTGTTACCTTATTAATTAGGCCTCTGTTGTAATTCACCTAGTG
CTAACCCAGGGATTATAGCATCCATCTTTCACTCATTACAGTCTACTTAAGTTAACTTAGTCCTTTAGTTAGCATCATTTCTGTCTTCCATACT
ACTGTATGCGTTTTAATTCTACATATATTATTAAAATTATAAGACATTTTTATTGTTGTTTTAAATAGTCAGTTTAAAAAATGTACCCTATATT
TACTGTTTTCAATGAACTTTCTTTTTTCCCACATTACATACTTCCATGAACACCTCCTTTTGGCCTATAGAACTCCTATTAATATTTCTTTTAG
TTCAGAGCTGCTGGTATGTATTTTCTCAGTTTTAGTCTGGGCTCACTTTCTGTGATTCTCTCATCCCTGGATTATTGACTTTCAAAACTCAGTC
ATATTGGCAGCCCCTAGCCCAGTGACACTGTCTCAAATCCCAGAACAATTTATTTAATGCCACATCTATTCTTGCTCCAAAAATTGGCAAATGC
CTCAAGAAGAAATGAAGAGAAGCCAGTATGAGACTCGCCTCAATCTACTTCCCTTTTCTGTGGGATCTTGGATCCCTCTCATTGACCAATATCT
TTAAAGAGTTAGATGATAGATAGATAGATAGATAGATAGATAGATAGATAGATAGATAGATGATAGACAAACAGATTGATGTTATTTTTAGCAG
CAGCATTAGTGTGAATAAGCTACTCCATTTTAGCTAGAGGTAGAAGTAATTTCTTCTTAGAGAAAAAGTTTGTATAGAATTGAATTTACCATCA
TAGCCATTCTTGAATGTCAGGCCAATTGAACTCCTAAGACTCTAGCATATACAAAAAACAGAAACCACTATTTCTTATCATATGTTTAAGGGTG
TGTATTTGGAACCAATTTAGCCACAAACTTCCAGATGATGCTGACATACCAATAATTACTATTAAAAAGCAATATAATACACAGGGAAAGCAAA
TATAGTCAATTGATATATGAGCTGTTGCTCTTATTGCAAGGATTGTAAGAGTTCTGCAGTGTGGCCATTATATGTGTGCCTCACAACTTGTATT
CATTGTAAGCTTCCTTTGAGAATTTTTATCTAAGAATAAGTCTACCCTCATTTTTTGTGGAGTATCAGAGCTTCTTCTCCTATTTGTCTTCAAG
ATTGAAAGGAGAATATTGAATGCTGATTCTCTGAACATCTGCAATAATGTATTCCAAGATATTGGCAAAATCAGTCCAAAGGCTCAACCATCCC
AGCTAATTAAATTACCTGGTTAACTCTGTGGACTACCAAATCTAAATGGATGTAAGAGAGGAAGAAATTTGAAAAAAATAAAGATTACACAGTT
GAAAAAACAAGATATTCTCCAGTTATGAATTCTTTTCCCCAAGAGTGTCTTTTGTGTACATTGTTATTACAAAATCCAGTTGTCACATTAAACA
AGCCATGCTATCCAAAGCAGAGTTGATTGTGGCTGGAAATTTATTCCAATATTGAAAAGATGTGAAAGGGCTATAACACGTATCAGACAATCTA
GAATTGACCACTAGGGGACATGACCATGAAGCCTTAGACAATAATTCATATTTAGTTTGAAGTACTATTCTGAGGCAAAACAAACAAAAAAGAA
TGATTATGGAGGACTCAATCTTAAAGTGTCTCCAAGCATGTCTTGTTCTGTCTCAAACCTTGGCCTCAACAATCAAAGCTCTGTATCATTATTT
GCAACACATGAATGCTAAATGGTATCATTTCTGACTATGGACCTATGTGTAGTTAAGTTGTGTGTAGGAATATGAGGATAGAAACTATTACTAT
CAACCAAATGGTAATATTGGACATGTTTCAAAGATTGTCTGAAAGAGCTTGTCCATGGAGACTAACCCTTAACAAGTCCCAGGTTTCTCCTCTC
CTAGAATGTGAATTCCACGTTCACCAGCAGAATTCAGTTCTGCTGAAATAGTCATGGAACAGTATTGTTTTAATTTCTCAAACTTTTCAACCAT
ATGTTTATAAAGGTTTATAAGACAAATAAGACTATCCCTGGCATATAATTCTTTCAGTAAATACTTCACATATTCTTCCATAAGAATTAGCCTC
TTCTGTAAACTCTGGAGCTACCCCAAGGTAAGATCGTACTACTGTAAATAAGCCCGAATCACTAGATTCCAAAGGCCAATGGTTACCCAGGCAT
TATTTGTTTATGACCTAAGAAAATAAGTCCATAGAAATAAATTGGCTTGAACAGAACCATCAAAAACCCCTTTCAATAGACATCTCAAATGAAA
TAGGTGGTTCTACTTCCTGAGAACACCTAAAGAACCTTTGTGTAAAAGAAGAAATCATTATTTCCATTCAGTAGCAATTTTTGAATGGTGTAAC
TTGCTATCTCCTTGCCTATTGGTACAATGCAGAGAAACTGTTCAAAGGTGAGCCATTCGGTAAAGAACGATAGAGTTAATCAGAGAGTCTCTGT
TATGCAGCTACATGGAATGCAGGTGTTCATTGCTGGTGTTTTTACCCGCTTTGAGTTCTTGTTTGTTTTTGTTCCTATAGTAGGATGCTGCACT
GATTTGGGCAGGGCAAAGTGGTGGTTGATGGAAGAGCAGAACACCTGGGAAAGTCATCCAGAGTAATTTTTGATCCCAAGAAAAGATCAGCAGA
ATTCTCCTTGAGTCTCTAAGTCTTCACATTGTAGAACTGCTTAGGTAAAAACTAATTCAGGAAATGTGCAGTTATAAAACTTTAATAAGTCACC
CATTAACTGACATTTTAAATGTAAATGTACTAAGGGGACTCTTTATTTCCCTCTCATTCGTAAGTGGACCATCCTCAACACAGAGACCCAAACC
AGAAACTTAGGAATCATCTTTGATTACTCTCTTTCAATCTTATCAAATCTTATTAGCAAATCTTATCCCTGAAATCTTTGTCCCTGAAATATAT
CTCAAGTCTTACCTGTCCTCACCAGTTCCACCGCTACCATCCTAATCCTGGCATCAACTCTGAACTGGACTGAGTCCTCCAGCAGCCTGGTTCC
ACTGCTTCTATTTTTTTTCCATTGTCTCTCTCCATTTCATTGTACACACAGTTGCCAAAATAATATTTTAAAATCTGTATCACTAATTTAAACG
CTTGTGGCTTTTCAGTGTACTTAGAAGAAAATCCACACTTGTAGACCCTGATGCATTCAGCCCCTACCTGCCTCCCCAGTCTTATACCAGACTC
CATTTCTCATATATTGCAGCCATGCTTGCCTTTTCAACATCTCAAGCAATCGAAGTGCTTTCTCGATTTGAGACTTTTTGTGCTTGCTATTTTC
ATTGCCCGGAATGCCTTTTCTGAGAATCTCAAGCAGTTGACTCCTCTGCATTTTTTATGTATAAGCTTTACATGGTTCATATTCAGAGATCCCT
TCCCTGAGCACCCTTAGTAAACTGTTCTTGGCTTTGTTGGGTATTATTGGAACTTACCACTGCCTATAATTATTATGTTTGTCATCAGGTTTAC
TTGTTTCTTGCCTGTGTCCTCCACTAGATTGTAAGCCCCAGATATCAGCTAATTGGTTGTATAAGTTTCCATTCTTTATATAGTCTTTGAATAG
TAATCATGTACTTAGTAAAGCCACTAAGGCAAGTGGCCAAGGAGACGTGGACAGTTATTTATATCTAATCCAATCTGAAGAATTATTCCAAGCA
AAAATGACGTGACTGACGGCTTAAAATAGCTAAAAACTTATTTAGTTCTATCTCAAACCTCAGCTTCAATAAGCAGGCTTTCAGCCACATGTTT
TCAGTATATGAATTGCCAGAGCTTCCTACCAACTAGTCATATAGTGTGTCTACCTTAACCTGGTACCTTTACTTGGGTTGCATTTCATCAGCAG
CCAAGTCCATCTATTTGTCACAGTGTCTCTAGTACACTGCTTCTACATAGTCAGCAATCAGTGAATACTTGATGCCTGCATGCATGAATAAATG
AACTTCTCAGGAATGCTAGGGCCAGAGAAAGTTGATGTTTAAAAAGTTAGAAAATGTAGGCACTTGAGAAGGATTAAGAAATTAACATAAGCCT
TCTTTGAATTTATCATTGAAATCTATTATTCAATTTACATGTACAATTTCAGCAATACATATACTTCCTTTAGTCAAATACATCTCCCTGTAGC
ACTTACCATGTCTCTGACCCTTTGCCTTGCAAATTCCACTCTGATAATACCAGCAGTGAATAGAAATTTAAAAAGCCACTGTCCTCACTCCTTC
CACTCATGTTCTAATGTAAGCCTTTCTTTTATGCAGCCTGAATGCACAATTCACTTGTCATTATCTCGCATCCTAGCTGCTCCTCATGAAATGG
CATTCCTCTGGATTTGCTGAGTCCTAATGTTCAGGCAAAGTAAATCCCACATCATTCTGGCAGGCTTTCAGGAAGACTTAGGGACTCGTTGTCT
GCTAGTGCAAATAACAGCAAAGTGTGGTAGGACTTCCTCCTTAGTTCAGCGAAGAGCCAGGTCCTTGTCGCACGGCCATGAAAAATTAGGCTCA
CAGACGATTTCGAGGGGGAGAAAAATGAAATTTATTGGGCAAAAATGGGGAAATTAGAAACAGGGGCCCTCTCCAAAGCCAGAGTCCCTGCTAG
AGTACTTCCCACCTCACAGGTTGAATCCCAGGTGCCACCCAGGAAGAGGAGGGGCCAGACTCCTCCCCTCTGCCAGCAGAGCAAACTTCTGTGG
CTCCACCCCAGTGTGTATTCCTCCCAGTGCGCAAGTCGGTTGGAGTTTTCTGGGAACCGCCTTCCCACCTGGCTGTCTCAAAAGGACTCCCAGT
TGCAGAGGTATGTGTGATTCAACAAGATCCACTGGCAGAAAATAGAGGCAAATTAACTCAAGGAGTCCATTGGAATATATCAACGTACTCCCCT
TCCACTCACAAGGCAATGATTTCTAAAACAGTGAATCATTTTACTTACAAGGGGTCACAATGGAGACAGGATAGAGGAAACTGCCCGGGAGACA
CAGGGACGAGGCATGTGGAGAGGTAGACCTTGGATCCAGTTCTGCAGCCAGTGAGCTGCTCACTCAGACCTGTTGCTCCCCTCCAGAGCCTCCC
TTTCTTCATCTGGGGTATGAAACAACTGGACCAGCTTATCTCTGAGATCCCGCTCAGCTCTGACCCTCCTGTTGGAGAAGGCTTGCATTCTGAG
ACTGCAGTCCCAAGTAGCAATCTCTTCGGAGGGCATGTATGTGATTACGTACTTAGTGTGCTCACATAGTTTCATAAGAATCCGGTAAAATTCA
TTCTTATGCTACTTTTGTATTCCCACTTATTTTGAATGGAAAATCCTTGTACATATTTCCTCAAATTATGGATAAAAAGTATACAAAATACACC
CTAGAGGCCAAGAAGCTCCTTTTGGTAGAAAGAATGTTGAGCCCTATGAATTTGAGCTACTCAAACTTTGTGGTTTGTTTTGGGAGCCAATCTT
GAATATAAAATATTTTTTGATCCAGCAGAAAGGTGAAATCACATTGAAGTTTACAAAATAGTAATGCTAAGATAGATAGATTTTCTTGTGTGCC
ACACTTATGTTGTAACCAGAACTTAATTCACCATAATCATTGACAAGTCTTTTGTTACTGGAAAGGATAAGGGTTGGCAGGTCTTGTGACAAAT
GTTAGAATTTAATGCCCAGGATTTTTCTTGATGCACAGGACTCATTGGAAAAGCGGCAGCTATTTATTGTGACTATGAATCACGTCCACAGATG
AATATGATTAAGTGCTAAATCCATAGCAGCTCAAGTTTTTTAAAGCCTTGGAAAATTGGCTTTAAAAAATATCACCTGACAAACCTCAAGCTTT
TTCTGTCTCACAAGACTTTTGGAAAAGAACTAGGATCGACAGTGTTTTAAGGCTCACAAGGAAAAGAATCTAACACTTGTAAAATTATCTTCTG
TGCAAGTTTTATTTTCCAACGTGAATCTGGGCTGCCAAGCATACTGGCTCTAGCCAGATCTTCATTAGCCGATCCAAAAAGACAGTCGGGGCAT
TTATCCAGATAATGCAAGCCAACACTACCCTCTGAGAAAACAAAAAGTAACTTGCCTGATGAAGCAAGGAACCTCACCGATTATTCCAGATTAA
TGCATCTCCTTTTACTGGCACCATGATCAGATATTTTCAAGACAAAGTACTTGGCAAGTTGGAATCCAGCTGGGTCCTTTGATTTTTGAATTCA
CTTTGCTTCTGTGATTATGGCCACAGTTTTGAAAATAAAGTCATGTGGATAGAGTTGTGATTTTTAGGGATCAGGATGCCTCATAAAACAACAA
TGCTGTATTCTTTTGTAGAACAAGATCCTGCCTAGGCCTCTTTCCTAGTATTAATCTTTGGTCATGATGAATGTGCTCCCAACCATCCAACCTA
TCCCTTTGCAACCAGGCTTTTGTGAGATCAAATAATTTTTAGAGTTGAAATGAAGTTATTACCAGCTCTAAAATAAGGTACTACAGGAGGGAAG
TAAGGACCGACAAGAGTTAAATCACTTTCTAGGGTTGCGCAGGTAACTGGGAATGAAGCCAGGATGACAACTCTACTTGCTCTTTCATTGTATC
TTATGCTTCTCTGATGTCTTCCAAGGGATAGCAATATAATCAATAGTCTTGAAAGAAAGATCTTTGTAGAGAAAGGGTCAGCAGATTATGTTTG
TAATTGACTAGATAGAAGTAGTTTTGCTTTTGCAGGTTAAATGGCCTCTATAAAATGTAATTACCCAATGGGTTCTTCCTGCCCACTACACAAA
GACCACAGCTCTGCAGTAAAGAGTTTAACTGACACAAGGCCGGCCACGCCATGTGAGAGACAGAGTTATTACTCAAGTCAGTCTTATCGAAGGC
TTGTGGGCTAGTGGTTTTTCAAAGACAGTTTAGGGGAAGAGGTGGGGGTGGCTAGGAAATGGGTGCTTTCCACTGATTGGTTGAAGCGGAGAAG
AAATCGTAAGGGGTCAAAGCTGTCCTCTTGAGCTGATTCACTTATAGGTGGGGCCCTAAGGGTGATTTGGCAGGTCCGGGTGAAGCCATCTGGT
ACAAGTGGAGCCAACCATGTCAGACATGCAAAAAACCTGAAAACATATCTCAAAAGGCCAATCTGCAATAGTGATGTTATCTGCAGGAGTAATT
GGGGAATTTGCATATCTTGTGACCAGCCTACACCTTAGCAGAATTCAGGCTCCTCTCCTCCCCATAGACTGGGGCTCTCTCATGGCTTTACAAA
GGTGGTTGACTTTTGGGGAGGGGCTATTATCATTTAAACCATAACCTAAAAGTCTTCCAAAGTTATCTTGGCCTCAGCCCAGGAGTAATTAAGG
CAGCTTGACCACTAAAGGTAAGAGCAGAGATGGCTAAATCAGATCTCCTTCACTGCCATAATTTTCTCACTGATACAATTTTTGCAAAGGCATT
TTCACCCACTACCCAACTGTGTCATTGTAGCATGAAGGCAGCCATAGAAAGGAATAGGCATGACTGTTCCAGTGGAACTTTATGAATTAACACT
GAAATGGAAATGTCATACAATTGTGACATGTCACAGAATATTTCATTTTTAATTGTTTTCAACTATTCAAAAATGTCAAAATCATTTTTAGCTT
GGTGGTGGACTGGATTTGGTCCAGAGCAGTAGTTTGCCTGCCTCTGTTATAGAGTAACACATCTGAGCCCATGAGAGAATTGATCCTGTACCTT
GGGTAATGCTCTTCCTCTCTGGCTCCTTTCTACACCTCAGTCACTTTTCCCCTGCAAGAGTGTTGTCACATTAGACTGTAAAATTGGATCACTT
TTATATTTCATCTCTAACATGCCTTATTTAATAAAAGTCATTCTAGGTATGACTAAAGTTCCAGGGCTAACTTAGAAAAGGACTGAAGAGAAGA
ATTCCTTCTGACAAATGCGGACCCAGCCACAAATGCCAGTGATATTGTCCCAAGTATTGGTGGCTTGGGTTGCACTTGATAACAGAGGGCAAAA
GAAAGTATAAATTTTGCTTGTTAACATAGCTTTAGTGGAAAATACAGAAAATGCCCCCAAACTCCATGCTAAGATCATGAACTAAACTGTTACA
ATGAAAGGAATTACTTGGGTTTCTATAAAAACAGCTGCCTCAAGGAATTTGCCACATTCCTTGGGTCTGCCTGAGCTCTGGTGGCCCACTGCTT
GGATAGGCCTGGTTTGTTTGCTAACTTAACATGCTTGCTTAACGGCCAAACATTCCCTTGAAATTCAAGGACTTTCTGTCCCGCTGACACTGTT
TATTTGTATCAGTAGAGTGGAGGGCTCTCTGGTGACACTCTAGTTTTTTCTGGGTTATAAGCAGATGCAGGTCTGTAGTTTTTCTGACTTGGAT
GGGGCCAATGGGTCTAAGCCAAGAGGAAAATGCTTTTTTCTTTTTCGTGTTTATTTCCTTTTTTTTTTTTTTCCTATCTTGATTGGAGTACCCT
TAGAAGGAAAAGGAGAGCCATAATCGTTCTCTGGAGGCCAAAAGAAGGCCGAAGGATGCCAAAAATTGTCACAGTGGTGACTTTTGGCTGGAGC
CAGTGCAGAATGAGGGGCTATGAGTGTGTCACTAGAACTGTAATCCAGATCTCTTCCATGAGGTCAGGAACCCTGATTCGTATTGCTTTTTCCA
TGCTGTAGAACATGACACCCCACTCAATCACTTGTGCTGGTTCATTGTTACTTGTTATAGTAGTCATTGTTATCAAGACTTTTTGGTTGTAAGT
GTCAGAAACCTAACTCAAACAGAGCTAAGCAAAAATAAATCTATAAATAAATATAATTAGCTCAAAAGTCTGAACTCACTGGATTGATCTTCAG
ACATAATTGCATGCAGTGTTCAAGCAGCAGCTACCATCTCTCCTTCCCTTCCTCTCTCTTATCTCTGCTTTCCTCTGTGTTAACATCCTGCTCT
GATAAGCTTTCTCCATGTGGAATGAAAGACGGGCTCCAGCCTTTCCAGGCTCCTAAGTTCCTTGCAGCTCACGGTCCCAGAAGGAAAGAGATTG
TCTCCCAGCATCTGTCTCCATAAAAAGAAGCCACCGTCCTTGCTTGAGTCATGTTCTCACTCTTGGACTTAGCAGAGTCCAGAGGGGCTTGGAT
ACTCTCACTTGGATACTCTCAATACGTGCCAGCAAGGGGCACATATTCACCCTTCATTGGGCAAGGTAAGCCTGTTAACAGTCCCACTCGAATC
ATGGGGAGTAAGCAAAAGGAAGTTCTGAAAATGAAAGAATGTGGGACAGACATAAAACTCAAAGCTGTGCACTATGCTAGTAAATTATTCTTGT
GTACTTAATATGAATATGGCTTTTATAAACAGTAAGCAAATCAGAAGCCACCCTGTGAATCATCAGTTATCTCTAGACATTTCTGGATTCCTTC
TTTATTCCCTGATAATCTTCTTTCCTTCCTTTCACTGCGTTGTAAGTGGAAATAACACAAGCCAAAAAGACCCTCTCCTGCTCTCAATACACAC
ATGGTCTAGTTCAGGAAGACAACATATAAACAGCAGGCATACGAGCAATTTCAGTAGACTGTGATGCCTGCCCAGGGTGCTCGTGGTCTACCTT
TCATGGACCAAAGACAACAGGAATAAGAGCATTCATCTTCCTCTGCTATTGTGAGAATATAGACAGTGCAAAGACTTGGATGCCAGTGCTAACA
TCCCTGCCTGACCTGACTTTGAGAAAAGGACCGCCATTTTCAGCACAACCCACAGGTTCTCTAAATACTCAAAGTGTGAGGCTAGCAGATAAAT
TTGGCTTATAGAGGGAGCTTCATACCATGGTTAGAACACAGCAAGAAAGGAACAAATTTTTAATGTATTAATTGAAAATCAGATTTCTCAGCTG
GGAAGAAGATATTAGCTGGAACACACACAAAAAAGTATTTTTAAAGGTGCCCTTTTAAAGGCTGCCAAAACTCACCCAAAGTAGTATTCAAATA
TGAAGACAGATGTCTTTTCAAGACGGGAAATTTTAGTTCCGAGTATGAAAGAGAATTTTATTTATTTGTTTTTTCCCTTGCCTTTTCAGGCTTT
TCACTGACTTCGAGGGGATGAGGGTATGTGTGCTTAAAGTAGATGCCAGAGTGTCTTCTTGGAACTTCAGGTTTCCATGAGTAGAAGGAAGTGT
TGAATGAATGAGGACCCACGGCCCTGTCCAGTGTGGACATGCCATAACCCTGTGCTGAGCTTCCTCATCTGTGTTCTGGGCTTACATTGAATAT
GGCATAAGCAACCCCGAAGAGCCACAAAACTCTGCTCTGTTAAGGGAGGAGGGCAAGTCCTCCCTGGAAGGGGTTGTCTCATCAACCAGGAAAT
GGTGGTCAGGATGGATACAGTAGCATCTATACCTCAGCCCCCTTGAAGTATCCTGATGTCCACTCCCCACAAAATCTAGGCCTTCCATAGGAAG
TCAAGAGGATATTGTGCTGATATAAATTTGGACCACCAGTGAGTTTGCCCTCAGACCTGGTAAGAAGTGCATATTTCAGATATAAGATATTGCT
CTGGCCTCAGCTACATACAGTTTCCATAAAGGAAAACTTTGAAGGACTGCCTGCAGGAAAAGAATAGCCTCGTGTTGATGTATCTAGGGACAGA
ACTCTCAGGCAGGACAACAGCTTCCTCAAGTCTTGTGACTACACTCTTTTTCCCAAGATATCTCATTGCATCTTTAATTTAGCTCCTAATTCAA
AGCCAAGCTCATGGACTGTTTCAAGCTCTGGACCATGAGCTTAGCTCTGAATTAGGAGCTAAATTAAGGATGCAACGAGACATCCTGGGAGAAA
TAGTATAGTCACAAGACTTGAGGAAGCTTTAGTGAAAAGCAGACCCACCCAGCATGTGTAATTCTTCCATGAGGTTTCATCTCACAGTCTGCAT
GTCTTGCAAGTGGGCTTAGGGCCACAGGGGCAGGAGCTCTTGGTACTCAGAATGTTGTCTAGAAGTTTGGAGGAGAGAGATCCCTTCCAACCTA
GGTCAAAAGGCAGAACTGGTAAAGGACTTGCAGACATGATAACTTTGAGCACAATATCTGAGAGAGCAATCCATGGAAGTCATTTGAGGAAGAG
CATTGTAGACAACAGGAATAGCAAGTTCATAAGCCCTGACCTCAGGAACCTGCCCGGTACATTCAGGAGCATCAAGGAGACCATACTGGCCAAA
TGGCACAAGTGTGAGGAAAGTGTTGGTAGGAGGTGATTTCAGAGGGATGCCTGGAGGCCAGATCCTGGAGAGTCTTGTAGGTCATTGTATAACT
TTGACTTTTACTTTGAATGAGATGGTAAGCACTTATGAGCAGAAAAGTGTATGATTTAACTTACGTTCTAAAAGAAGTATTCTGGCTCCTGTAT
TAAAAATAAACTGAAGGGCAAGGAGAGAGGTGGAGAGACCCATTGGGAGGCTAACGCAGACATTTATCCAGGCAAATATCACTTAGACCTCGGT
GGTGCAAGTAAGGAAAATGAGAGGAAATTGCATTTATAAAAATCTCATTTAAAGATAGAGTCAACAGGATTCATCATTGGATTGGATTGGAGTA
AATGAGAGAGAGAGGGAAATCAAGGATGATTCAAAGTTGGTTTTGTTTTTTTTTTTGTTTTTTTTTTTTATCTTGAGCAACTGGAGAGATGGAG
ACAACATTTTAAATTACTTTGTTCTTTGGTAACATGGCAGTAAACACACTTATAAACTGTATTACAGGTGGCAATCAAAATAAGCACCTGGCTG
ATTTGAAATATGGTTTAAAAAAAATTACAAAGTAAGGAAGATGAACTTGTATTGTTCTGCCCATGCAAACAATCTGTAAAACTGTGTGCATTTA
TATATTTTTCAGTCATTAATATTTACAGCGGGAGAGAACTGTTTGTGAAATCATGAATTATCTTTTGTTTGAATTTTATTTTATTAATGAGTTA
GAAGTACCTTTGTAAGCCATTGTACCTTTTTTCTAAAGGTGAGAGCCTAAAACTACCATGTTGCAATGGAATAAGCCCTGGTTCTAGCTTCACG
CCCTGGGAGTGACTTCTATTAGCTTTGGAATCTCCGGTAACTCCTTTAATCCTTTGAATTCTCACTTTCTATGTCTTAAAGGTGATGGCTGCCC
ATTTCATCAACTGTGAAATGGCATCCAAGTCCCAGGTATCGTTAAGAACCAACGGCTTCCATTTTAAACAGAAGTTTTGTTAAACAAATATTTG
GTTTTACAAGGGTATTATAATTCATACCCTTATTCATTGTTCTTCCTTGCTCCCAATAGTCGATGGGTTTTTTTTTTAGCTGTGGCTTATGTGA
TCAAAGAGGTGAAACAAGTTAGCTCAATTGGTTATATTCTGAAATTAATAAGGTGTTCTTTTTGCATGTGTATACAAATTAACTTCCCTCTGTT
TCTTGGCCACACACCTCACCTCAGCTTCACAAATGAGTAGAATTGATTACAACATGAATCAGACAAGAGAGTGTAGATCTATCACTGTTATTGA
GAAAGCAGTTCAAAAAACAAGCATTCCCGCATGTTAGTAACATGTTTTTATAAAAGCTGTAAATGTATATTTATTCATATGATTTAATATTTTT
TATCTTCAGTTTTTATTTTAAGTTCAGGGGTACATGTACAGGATGTGCAGGTTAGTTACACAGGTAAACATGTGCCGTGGTGGTTTGCTGCACA
GATCCTCCCATCATGTAGGTATTAAGCCCAGCATCCATTAGCTATTCTTCCTGGTGCCCACCCTCCCCTGAACCCCCTCCCCTGACAGGCTCCA
GTGTGTGTTGGTCCCTGCAATGTGTCCACGCGTTCTCATCATTCAGCTCCCACTAATAAGTGAAAACATGTAGTGTTTGGTTTTCTGTTCCTGC
ATTAATTCGCTGGGGATAATGGCTTTCAGCTCCATCCATGTCCCTGAAGAAGACATGATCTCGTTTTTTTATGGCTGCATAGTATTCCATGGTG
TATACGTACCACATTTTCTTTATCCAGTCTATTATTAATGGGCATTTGGCTTGATTCCATGTATTTGCTATTGTGAATAGTGCTGCAATAAACA
TATGTGTGCATGTATCTTTATAATAGAATGGTTTATATTCCTTTGGGTATATACTCGGTAAGGGAATTGCTGGGTCAAGTGGTATTTCTGCCTC
TGGGTCTTTGAGGAATTGCCACACTGTCTTCCATAGTGGTTGAACTAATTTACACTCCCACCAACAGCATAAAAGCCTTCCTTTTTCTCTGCAA
CCTCACTAGAGTCAGTAATATTTCTTCAAATATGAAGGACTTGACCTCAGATCCTTTATGATGATCATAAATACATAAATAGATAAGTTAAAAA
ATAGGAAGGACAGTGTGATACTAAATAACTACAAAATTAGAGATGTATCAAAAAGTATTTAGGCACTAAAATAGTTGAGAGATCATTTTCAGAG
CCTCCAAGAGTTTCTATGCCTTGAATTATTTGAGCATCCAGTGCAAGCTTTCAGAGTCCTTGCTTCCTTAAATATAAAACAAGAAGTCAGATAC
CTTTGCCTCTGACTTCTCAGAGGTCTTTGAGCTTGACAGTTCTATAATTCTATGAGTTTTCTACCTAGAAACTATTCCTTCCATTCAGTTAGGA
AATATATTCAAGTATATTTTATTTAATCTTAAACTTTGGAATCCCATTTTGGTAAGCTGCTGTTATTTGAAGGTGTTGACGTATCCTCTGGGAT
CTGAGAAAATTATTATCAAAACCAATTAAAGGAATCATCTATTTTGTGCGATTGGTTTTGAAATCTGTCCAGTGGGCTCTTTCTGGAGGCAAGG
ATCCTGGTTACCAAGAGACTTTGCTGCAAATAAGGCAAACTGGGATGACATTTGGGGTTGGGATCCTTCTCAGGACCAAATGGGCACCCATCAT
CTTTGGGTCTAGAAGACTGCACTGGCATAAAGCAGCTGACTTCCTGTAGCCTTGCAGAAATAACAATGGGGTGTGTGTGTGTGTGTGTAGTGTG
TGTAGTGTGTGCAGTGTGTGTGTGTGTGTGTGTGTACAATGGTGGGGAACAGTTGGCAGAGCAATGAGCAAGAAATGGGAAACGACACTCAGAA
AAGCAGCAACCTACCCTGAGCAAATCAAAGAATTTCTTAGAATGCAACACTAGCTGTGTGTATGAATAATTGGCTTGCAAATCACTTCCTCTAT
TTGCTTGTTATATAATACCAATCATTTCAGTGTCACTGACGAAAGCCCAGAAGGGAATTACCACTTACCAGCCAGTTTTACATATGGCTTTTGT
GAGGAATAAGGCCTTGAGGATGAGGGTCTAAATCTCTGGCCCACAGATCTGGACAGGCAGATTTCAAGCTGATCATTACATATCTTTTGAGAGA
GACAATCCCAGACGAATTGGCCTGGGGGGCAACATGAGAAGGCAAGACAAAAGAACATTGTAAAGACTTCACCTAGAATCCAAATTGTAGTTCT
CTCAGCAAGTTATCAAGAAGCAAATTAACTGCTAGATTTTGTAATCCAAAGATTATTTAATCTGTTTAGCTTTGTGTTGGAAGGATCGCTACTA
ATCATCTCTTTTATTAAAATATGATAATGATAGCCATCCTTTATTAAGTGCTTATGATATACCAGGCACTTTGTATACATGATTTGCCTTATTC
AGAATAACCCTGAAGGATCAATATTATTGTCTCCATATCAAAGAGAGGGAAACTAAGGCATACAAAAGTTATTTGCTCAAGGTCAAACAGCATG
GTAAGAGGAACTGCAATTTCATTCCAGGTCTTACGGATACCAAGTCCGATGCCTGCCAGCTTGCACCTGTTAAGTCATGTGATACTTAGGAAAT
GTACACATCACCCCAGTGCAGAGGCCTTTATCCACTTCCCTTCATTTTGCCTGGATTGTTGCTGTGAGTGAGAAATAAATGACATGAAATGATC
ATGTATATCACTGTCTTCAGAGGATGGTTAGTTCCTTGCTCTGGATTGTTTTTCTCTTTTGTTGGATATTTATTTTGACTTCTCTGCAAACTAA
AAATATGCATGCTCTAGTTTCTCTTGGGTACATAACTCCCAATATATTGTGAAGAATGAAACTGGCCTACAAAGTTTAATTAAGAAGCAGTGAA
CTAGAGCTTACTTAGACAATACTTGTACTAGTGTGGGTTCATGTTGTCCATTTGTCTTTAGAGGTGTAGAACCTTGAAAGTCAGCTAATTAATC
ACTATTACTGTTGTGTACAGTATTGGCAATCTCCTATGCTAAAATTTTTTGTGATGATTTGAAGGTGGGAGGGGGCAATATTTACTAAGTCTCT
CAACCTACATGGCAATATCTGAAACCTGCTTATAATGTAGTAATAGCTTAGGCACCACCTATTTCTGAAACATAAATCATCCCACAATATAAGC
CTCCCAGTTTTATTCATGCGTCTTACCATGTATAAATATCTTTTAATATACCATTTATTTCCTTGAGGGACAGTTATTTGTAGCATAAATCTGC
CTGGATAAAATAGTAAAAATTGCTATGCCCCAGTGAATTCAGTTTACAAAAGCTTACATACGTGAATGAGACATTGGAAATGTGGTTTATAGTT
AAGGAGAATGGATCACACGGATTATACCTGTATAACTTCCCTAGAAGGTTGATACCACTAAGATATTAACACCAAAAAACTGAAAATAGTATTT
TTCATCTTACTTTGTTATGTACCCTTTGTGAATTTCTGAAAAACACCTTTCTATTATGATGGGATAAATTACGGTGTGGTAACAAACAAGCCCA
ACCTGTTTGTTCCGGTCAACTAGTTGCTTTATAACAAACCACCCCAAAATTTGTTTAAAACAGCTATCATTTGATTGTGCTCACAAGTTCTGTG
AGTTAGGAAATAAGACACAACACAGTGGTCTTATGTCTGTTGTGTAATGTCTGGGTTCTCAGCTGTGAACACTCAAATGGCTTGGAAACTGAAA
TCAGCTGGAGGCTTCCTTATTCATATGTTTGGTGCCTGGCTGGGATGACTCAGATTAGTTGGGGCTCTTGACAAAAGCACCTATACATGAACCC
TCTCAGGAGCCTTGGCTCTATGGTGGCTGGATTCTGAGAGGGAGCATGCAGAGAAAAGAATTCCAAGACAATGGATGCTCTAAGAGAAGCTACT
TGGCCTTTTCTGATCTAGTCTTGGTGGTCATGCAGCATCACTTTTGCCACCCAGATCAAAGGGGAAGAGGAGACAGACTCCACCTGTGGATGGG
AGAGTGTCAAATTCACACAGAAGTTGTATGCAAGCTATTCTTGGTCATCTTGCTCATCTATGAAATATACCCTCCTCCATGCATTGACTTACAA
CAAGGGTTTATTTCTTGCTCAAAGTCTGCTGTGAATCTAGGCAATTCTCCAGAGCAGCTGTTCTTTTGTTGGCTTAGCAATCCAAGCTGCTGCT
TCCATCTTACAGCTTTTCCATATCAACTCATGATTCCACAACCATTGCTACGAGGCAAGAGTAGTATGGAGGATCACACACTACTCTTAGAAAC
CACCAGCTGGAAGGGACTTATATCACTCCTGCTTACATTTGATGGCTCACATAGCCATGCCCAAATTCAGTAGAGCAAGCATGGCCCTCCTGTG
TACCCAGAAAGGAAGGAGAGCTAGAAATATTGGTAAATAGCAATATTGTTTACCATAGCCTCAGAGATATTGCCTCGTGGTGAAACCCTATAAT
TCCCCTCAATTCTGATTTTTGCCCTTTATCAAATCATTTTATTCATTCCTTCATCCATCCAACAAAGATGTTTTATATGCCTACTATTAGCAAC
AATCAGTAGCTTGTTCCTGGAGGGAATGCAGCTGCTCCCCGCCAAAACACCATATAATTCACTCTAGAAAACTAAAATTTCAGATGCTTTGTCA
TTGATTATTGTGGGGAGAAAATGTCTCTTTCAGCTCCGACTTTGTCCTGTCTCCTCTAAGGCATGTGAAATAAGATTTTTTTTTCCTATTCTTA
CCTCCTATGGTCCCATTTTATGGTTTTCATGCACTACTGTCAGTCAAATATTTATCATATGTCTTTCAATATCTTTCCAATATTTATCATATAT
CTTTCCCCTTGTAGGCCTTAACTTTATGACCCACTATTAAGCACGCAGCTGTATTTCCAGAAAATGTAGATGGTTTAATTCAATACTGCATGGC
AATTAACTGTCTTATAAATTAAATCATTTTAATCCATCTTAGAGTTATCATGCATAATTAGTCTGACAGCACAATTCTGTTTGTTGTTGTTTTA
AAGGAAATTTAGATATCAAGGCTGCAAAGAATGCGTAAGTCTAGGCAGAAGGCTCTTATTTTCCAAAAGCAGAGCCCATCGTTGCATTTCAACA
ATGACCGCAACAAAAACCATCAGGAGCTGTTTGGGGCCTGTTTGTGCTTTAGTCTTCAATTAGAAAAAAATGTGTTTCTGATGGAAAACCATAG
AACAATCCTCACTCTGTGGTAAGAAAGAATAAAGAGAAAGAAGTGGGCTCTATGATAGACTGAAGCAAGAATACTATTTTAGACTAGGTAGTGG
CCGGGCGTGGTGGCTCACGCCTGTAATCCTAGCACTTTGAGAGGCCAAGGTGGGCGTATCACCTGAGGTTGGGAGTTCGAGACCAGCCTGACCA
ACATGGAGAAACCCTGTCTCTACTAAAAATACAAAATTAGCTGGGCGTCGTGGCGCATGCCTGTAATCCCAGCTACTCAGGCGCACTGGCTCAC
GCCTGTACTTGGGAGGCTGAGGCAGGAGAATCGCTTGAGCCCAGGAGGCAGAGGTTGCAGTGAGCTGAGATTGCGCCATTGCACTCCAGCCTGG
GCAACAAGAGTGAAACTCTGTCTCAGAAAAAAAAAAAAGACTAGGTAGTTATCTTCCTGGGTGGTTTTGGCTATGTAGAAAATCACTCTTACTC
TCGATTAATTTATGTACCAAATCATGCTTTGAAATATCCCACTGAAAAGGTCTAAATTATTGATATCTTGGGTCAAGTTCTGACTTGAAGGCAA
ATAGTAGGAAATACAATGGCATTTATTGGAGGCACACACTACAAATATTGTAGTATCTTTAAGATGTGTTTAAATAAACAAGTAGATGATAAAA
CTATAAATTAGAGTCACTAAGACTCTCTAGCAGAGACCCTTACCTTTTGAAGTGCACCTTAAAATGGCAACAACACTTATCAGAAATTCCATAA
GAAGCTTAGTTGTCTGCTACTCTATTGCAGTATCATATGTGTGAGCTGCATGAAAACTGGATATTATTATTATCAAAGGTTATTATCAAATGGA
GCACAATTTTATTAATAACAATATTATTATTATCAAAGGTTTTAATTTGCTAAAACTATACAGCTTCCTTAGGTCTTCCAAACTAGGTCAGCCA
GTTAACCCTCAGATCTGGAGTACCACATTTGGCTGAGTCAGAAGAAATAAAGATGCTCTCAAAACCACAAACAAAACTGATAATAGACATTCTG
ATAACTTTAACTCCCACAGTAATAAATCTTGGGTCTTTTAAAGTTGGTCAGATGTACACAGAGCTGCTTACTAATTATACTAAAATAGTTACTA
AGCAACTCGACATTTTGATTTCCAAGAATTTCAGATAAAGATTATGAACTTTATTTATTTGGTGCTTTAGAGTCCAAGGCACTATTTTACAGCC
TAAAGCTGTATTTCTCAAGGCCATCTTCTTGCATTCTTCTAAAACTTAAGAGGGATCATCTGTCCTCAAAAATATAAGAAATTCTGTAAATGTG
TATTTTTGTGTATATATATATTTGATATTTAAAATTCTGTATCTATATATACTTCCAAGACCCCTTAGAACTTACACGGTACCTGTAATTTGTT
TCTTTTTTATACTTAAGTACAAGTAATGTTAGTGATACATTTAAAACTAGGAATTACCTTGTTTGCAATATCAAATTTTTGTAAAATATTGAGT
TAAAAAAGTCTCCTAAAATTAAGATTTGAACTATTATAAATGGCTTCAAAGATCCTTTCGTTTCTAAGAGAAGGAAGGAATTAGGATTTATTGT
GCATGTATTTCATACCAGGTGCTCATCTACAGTTATTGAATCCTTGCAACAATCGTATCAGGTAAACGCTGTTATTCTTCTCTTACAGATGAAA
AATCTAAACCTTCTAAAATGAAAGTTTGTCCAAGTTAACATGACCAATTAGGAGAGCTTGGATTCCAACCCAAATCTGTTCAGTATCAAAGCTC
ACTCTCTTCCATCCTTGCCCCTTCTCAGCTCACATAGTAGCTTTCAGAAAACAGTAGCTATCTACCCCAGATAAAATTAAGCCTTTTTTTCCTT
AGGAGAGGCTAGAAACTGAATCTTCTACCCCCTTTATTGGCATCACACAGGCACAAAAATCACCAATTCTCAAAAACCCTATACTCCCAAGTTC
AACCCAAATTGAATGGTTGGAATATTGTTGGTTTGTATGGAATAATTAACCCACATCTAAAAATTAATTATACAAAGTGAAGTGGAGATTGAAG
CAGTTTTCTAGATAATTAAGGCAAAAATAACTGCAATTTTTTTAAAAGACAGTTTGAAATATTTAGAAGGGTAGATTGCAAAATTATCCAACTT
AAGGTAGTCATATTCAGGGAGTTTAGCCAAAACACCTCTGAAGTTCATTGCCTTACAAATGAGTATAAAACTTAAACCTATGATTAATTCCCTG
TTGTTTTTCTATCTGTATTATACATATAAATATATTTTATATATTGTCCTCCTGAGATTTCAAGAGCAATTTTCTTCTAATTCTTCTAATTTCT
TCTATGGATTCACCATAGTGAATCCACCGACACGTCTACCCTGTGACAATCTTGTTCTCCATTTGATTGCATGAAAAATACCATATGAATTTGA
CAAATCATTGGTTTTTTTGTTTGTTTTTCTCTTTTGCTGCATATCATTGCCTATGCTTCTTGAGCACTAGAATATATTTGTGCATGTATAAACC
AAGGCCTTTCTTCTGAAAATTAATAGTACTTTGAATCTCTTCTATATTCTGGTTCTTTTAAACTGTGGTTGTTTTAAGTTTGCCTCTCTTGTTT
TAGGATTTATTCTTTATTTTGAGGCCCTGATTTTTCACAAAACATTTTTCTCAACGAATCCTGAGAACACTGGACAATCATAGCTTTTAGCTAT
CATGAGTCCCATTACCCTTTGGCCTCCAGTTAAGCTCTGTCCTGCAAGACATAAATTGTCTGATTATCAACTCCTACTTTGAGTGAAAAATGGT
TGAATGAAATGAATATGCAGTTCATTTCAACAAAGCAAGAAATTTGCTGTTTAAAATTGTGTTTTTATAATTCTTGAGATGGAAATATAGCAGA
CCTCTGTGAATCTGAGAAGGCATTTCATGCTTCCAGAAATGAGGGGAGAAGTGGCAATGATATTGCTCATGAACAATTGGAAGCAATGATACTA
GAGTATGCAGCAAATCTCAGGGAGATTTCAGTGGTACAGTAACTGGCCTCACAAAAGTGGGATTTATTGCATTAAATCTTTGCTGTCCTTTCTG
GCTGGTTTGATATTTAAGAAATTATTAAACATAAAATTGTATGAGCAGTTACCTATGGTAGGACAAAATGGACTGACCTCTAATGGCTTGGTAG
ATGGGATAATTTTGGGAAGAGAGAGTGGAGCTCAGTTCAGCATCCTACTCTCCTCCTCAACATCATTTCCCATTCTTGGCCCTTGAGTTTAGAT
AGGAGTTGGGCATGTGTGTGTGTGTATGTGTGTGTGTGGTGAAGTGGGTAGATTCCTGTTCCCAACCCAATTAGATAACTCCACTTCCATCTAT
CTTCCTTGCATCTTCTCCAGCTCATTTGCAAAAGAATGAAAGAAGCCTTGCTTCAAAAAGGTTTAGAGGGCTAAATAAGAGCACAAACTGGATT
TTCTGAAAACTGACCTGATTGCCTGGGTAATTAAACTGTCACTCCAGCAAAAGCATTTGTCTCCAAAAAGTACTGAGGTTGTCTTTCATCACTC
TAAGTCTAGACATTCTGAAACCTCCATCCTCTCCTGCAGAGAGAGGAATTAAGTCTCCACATTTCTAAACCATTGACAGTCTGTGGGGTGCCCC
ATTGTGTTCCTGACTTCCTATTCTCAGAAGGAGTTATAACGATGACAATGATGCATGCATGAGACATAGAGGTCCTCCCCAGCCCTCGGTTAGG
AAGTAAGGCAGCCACCCATGTACTGAACACAATAGTGCTTTGGAAAGAGCTCTGTTAAAAGTGAGTCTCAAAAATGTAAAGGCCTTTACGAGAG
GAACTTAATCTCCAGTGTGATAAAACGATGCACACATTACAAGGAGATGGATGTTATGAAAACAATAAGTACACCCAAGAGTACACGTACAAAT
CTGCATTGCAATTGATTTATTTGGATGCCTGTTTCTTCCTACACTATGAGCTCCTTGGGGCCATGAAATTCATTTGTTTTATCTATATATACTC
TCTTTGATATCCCCCACCCTGACAGCTAGTGCAGTCCTTTCTAAATAGTTGTTACTAAATATGTCAAATCAAATGCCTACAAGGACTGGTTGGT
AGAACCAGATTCTGGAAGTGACCAAGCAGGTCTAGCTTCATAAGAATGCCATCATCGTAGATTTGAGGGTCATGATGAAGTTGTACTAGGATTT
ATGGAGGTTCTTTGTCAACCAGTTCACACCACCAGTGACTTAAGGAAACTATATGTCGTCTATTGGGAACAGTCCTGCTTAAAGCAATTAGCAG
GGACAATTAATTTGGAGGGAAGTCTCTCTCCACTCAACAGCATTCAAATACCCTCTGCATTAGCCATGTTTTGAAAAACTCCAATTGCCTGAGT
CACTGATAGACAAACAGAAATGCAGACTATTTTGTATGCAGTTTTATAAAGAAGTTTCTAACCAGAGAAGAAAGGTGTTATTTTGAAATATTAA
GCTCTTAGTTGTTCTTTCATTTCAGTGAACATTTGTTGAGGGCCTGACATGAGCTAGGCACATGGCAAAGTTTTAGAGATGCAAAGGTAAATTA
AGACCCCTGTGTTAATTCTCATGCGGGCAAAGCAAGCACATTTTTTAAGTTGCAATAATGCAACAATGGTGGTGTGTACATAGTAGGGCAAAAG
CATAGAGAAAGCCATGGAAATCCTCAAGCCAAGTTCAAAGAGTCCTCTAATGTATGGCATACCACAACAAGGACCTTCAAAGGGTGCATTTGGT
AAGAAAAGGCAACCTCCAAAGATTCATCCAATCCTAAGGTTTCATAGAGTAGGGACTTCTTCCCTTAGCTGGAAAGGAAAGAAGTCATTTGGTG
TGCAGTAGACATAAATTAGGGGCCGTCCACTTTTTCTTGCAATGATTCATCTAATGGAGGAGCAGACCCTGGAGAGAAACATATCTCTTCTTGT
CTTCATTTTGTTCTTCTTTTATTAAATCACCCGCAGGACAACCGGATGCTCCAGCAATAGATTATAGAAAAAACAGATGGGTATTTCTGTGCAG
TCAGAAGGCTCCTGTGCCCTTTTCCAGCATAAAACGTTTATGAAAATCAATTTTCAGTGCCTGCTGAACCAGGCTGATGTGAGAACTAGCTTTC
CACAGTTCATAATATTCACTCCGGGATGATTTGACAGGGCTGATATTTTATCACGAATACAAAGGAGATGAGCACCATTCTCCTAATATATTCA
TTACTTGTCTCACCACCCTGGTAAATAATATGCATATGGTAATTTGAGAGACACAGTGTTCCCATTGCTCTTTTTTATTCCCAGAACTCTGGCT
GTCTTTTCTAAATGCTGCAAATCAAAGGCAAAGTTACCTCACGCACCTGTAGTCCCAGCTACTCAAGAGACAGAGGCTCTAGACCAGGACTTGA
AGTTCAGCCTGGGCAACACAGTGAGACCCTATGTCTTTAAAAAAAATTGCCTAAAAATTTGGCCAGGTGCGGTGGCCCATGCCTGTAATTGGAG
GAACTTTGGGAGGCCAAGGTGGGCAGATCACTTGAGGCCAGGAGTTTGAGACCATCCTGGCCAACATGGCTAAACCCTGTCTCTACTAAAAATA
CAAAAAAAAAAAAAAATTAGCCAGGCATGGTGGTGTGCACCTGTAATCCCAGCTACTTGGGAGGCTGAGGCATAAGAATCACTTGAACCTGGGA
GGCAGAGGTTGCAGTGAGCCGAGATTGTGCCACTGCTGTATTCCAGTCTGGGTGATAGAGCGAGACTCTGCCTCAAAAAAATTAAATTAAAATT
TAAAATAAAAAATTACTTAAAAATTTGATTTTACATTCCTCCTATTCTCACTTTCTGGTGCCCATACAACACATAAATTACATTCTTCTCTATG
TTTACCTGCATCTCTAGGGAACAAAGCCCCTTTCACTTGTTCTGTTACTATAGAATGTTCTACAATACCCCAATAAAGATTCATTCATTTGTTC
ATTTACAAATATCTATTAAGCACCTACTGTGTGTCAAGCAGTATGCTAAATCTTAAGCATGAAACAAAAGAAAGATAAAAGTATCTGATTCAGG
GACTGGTTGTTCTGTTGTTCTGGGTTTTTTTTTTTTTTTTTTGGAAGCTTGTTGAAAATCTGTGGCATCATACAAAACAAAAAAGAATGCAAAA
CTGGTGCCTTAAATAGAAATGTATTAATAATGAGTGTTAGAGCTAAGAGAAGGCAAAATGTCTACAGAACACATGATTTCAATTAACAACAGTG
AGTAAACGAATGATCTGGAAAAAAATTATGATCGGGCCCATGATTTTTTTTTGTCGTTTTTTAAAAATTTTGTTTGTTTTTCTCATACTCATAT
TATTCTAAGGCCCATTGATTTTAGAAAGAAAATCTGTTTTATAAACTTAGCATATTAAAAG
HUMAN SEQUENCE—mRNA
GGAGGCTGAAATCCCCAGACGCCGGTTTTCTGGGCTGGGCTTTCTGCTTACTCACTCCTTCTCCCTCTTTCTGGATTTTACCGACCGTTCGCGA
AACAGCTTTCCACACAATGGAGCTTCATGTCCTCGTGCAGGAAGTACTCATCGACTGATGTGGCAGACTTTGCTCCCTGACAAAACTAAAGAAC
TCTCCTATTCATGGAGGCGAACACTGAGGATGCTTTCCACATGAACCCTGAAGTGAACTTCTGATACATTTCCTGCAGCAAGAGAAGGCAGCCA
ACATGAAGGAAAATGTGGCATCTGCAACCGTTTTCACTCTGCTACTTTTTCTCAACACCTGCCTTCTGAATGGACAGTTACCTCCTGGAAAACC
TGAGATCTTTAAATGTCGTTCTCCCAATAAGGAAACATTCACCTGCTGGTGGAGGCCTGGGACAGATGGAGGACTTCCTACCAATTATTCACTG
ACTTACCACAGGGAAGGAGAGACACTCATGCATGAATGTCCAGACTACATAACCGGTGGCCCCAACTCCTGCCACTTTGGCAAGCAGTACACCT
CCATGTGGAGGACATACATCATGATGGTCAATGCCACTAACCAGATGGGAAGCAGTTTCTCGGATGAACTTTATGTGGACGTGACTTACATAGT
TCAGCCAGACCCTCCTTTGGAGCTGGCTGTGGAAGTAAAACAGCCAGAAGACAGAAAACCCTACCTGTGGATTAAATGGTCTCCACCTACCCTG
ATTGACTTAAAAACTGGTTGGTTCACGCTCCTGTATGAAATTCGATTAAAACCCGAGAAAGCAGCTGAGTGGGAGATCCATTTTGCTGGGCAGC
AAACAGAGTTTAAGATTCTCAGCCTACATCCAGGACAGAAATACCTTGTCCAGGTTCGCTGCAAACCAGACCATGGATACTGGAGTGCATGGAG
TCCAGCGACCTTCATTCAGATACCTAGTGACTTCACCATGAATGATACAACCGTGTGGATCTCTGTGGCTGTCCTTTCTGCTGTCATCTGTTTG
ATTATTGTCTGGGCAGTGGCTTTGAAGGGCTATAGCATGGTGACCTGCATCTTTCCGCCAGTTCCTGGGCCAAAAATAAAAGGATTTGATGCTC
ATCTGTTGGAGAAGGGCAAGTCTGAAGAACTACTGAGTGCCTTGGGATGCCAAGACTTTCCTCCCACTTCTGACTATGAGGACTTGCTGGTGGA
GTATTTAGAAGTAGATGATAGTGAGGACCAGCATCTAATGTCAGTCCATTCAAAAGAACACCCAAGTCAAGGTATGAAACCCACATACCTGGAT
CCTGACACTGACTCAGGCCGGGGGAGCTGTGACAGCCCTTCCCTTTTGTCTGAAAAGTGTGAGGAACCCCAGGCCAATCCCTCCACATTCTATG
ATCCTGAGGTCATTGAGAAGCCAGAGAATCCTGAAACAACCCACACCTGGGACCCCCAGTGCATAAGCATGGAAGGCAAAATCCCCTATTTTCA
TGCTGGTGGATCCAAATGTTCAACATGGCCCTTACCACAGCCCAGCCAGCACAACCCCAGATCCTCTTACCACAATATTACTGATGTGTGTGAG
CTGGCTGTGGGCCCTGCAGGTGCACCGGCCACTCTGTTGAATGAAGCAGGTAAAGATGCTTTAAAATCCTCTCAAACCATTAAGTCTAGAGAAG
AGGGAAAGGCAACCCAGCAGAGGGAGGTAGAAAGCTTCCATTCTGAGACTGACCAGGATACGCCCTGGCTGCTGCCCCAGGAGAAAACCCCCTT
TGGCTCCGCTAAACCCTTGGATTATGTGGAGATTCACAAGGTCAACAAAGATGGTGCATTATCATTGCTACCAAAACAGAGAGAGAACAGCGGC
AAGCCCAAGAAGCCCGGGACTCCTGAGAACAATAAGGAGTATGCCAAGGTGTCCGGGGTCATGGATAACAACATCCTGGTGTTGGTGCCAGATC
CACATGCTAAAAACGTGGCTTGCTTTGAAGAATCAGCCAAAGAGGCCCCACCATCACTTGAACAGAATCAAGCTGAGAAAGCCCTGGCCAACTT
CACTGCAACATCAAGCAAGTGCAGGCTCCAGCTGGGTGGTTTGGATTACCTGGATCCCGCATGTTTTACACACTCCTTTCACTGATAGCTTGAC
TAATGGAATGATTGGTTAAAATGTGATTTTTCTTCAGGTAACACTACAGAGTACGTGAAATGCTCAAGAATGTAGTCAGACTGACACTACTAAA
GCTCCCAGCTCCTTTCATGCTCCATTTTTAACCACTTGCCTCTTTCTCCAGCAGCTGATTCCAGAACAAATCATTATGTTTCCTAACTGTGATT
TGTAGATTTACTTTTTGCTGTTAGTTATAAAACTATGTGTTCAATGAAATAAAAGCACACTGCTTAGTATTCTTGAGGGACAATGCCAATAGGT
ATATCCTCTGGAAAAGGCTTTCATGATTTGGCATGGGACAGACGGAAATGAAATTGTCAAAATTGTTTACCATAGAAAGATGACAAAAGAAAAT
TTTCCACATAGGAAAATGCCATGAAAATTGCTTTTGAAAAACAACTGCATAACCTTTACACTCCTCGTCCATTTTATTAGGATTACCCAAATAT
AACCATTTAAAGAAAGAATGCATTCCAGAACAAATTGTTTACATAAGTTCCTATACCTTACTGACACATTGCTGATATGCAAGTAAGAAAT
HUMAN SEQUENCE—CODING
ATGAAGGAAAATGTGGCATCTGCAACCGTTTTCACTCTGCTACTTTTTCTCAACACCTGCCTTCTGAATGGACAGTTACCTCCTGGAAAACCTG
AGATCTTTAAATGTCGTTCTCCCAATAAGGAAACATTCACCTGCTGGTGGAGGCCTGGGACAGATGGAGGACTTCCTACCAATTATTCACTGAC
TTACCACAGGGAAGGAGAGACACTCATGCATGAATGTCCAGACTACATAACCGGTGGCCCCAACTCCTGCCACTTTGGCAAGCAGTACACCTCC
ATGTGGAGGACATACATCATGATGGTCAATGCCACTAACCAGATGGGAAGCAGTTTCTCGGATGAACTTTATGTGGACGTGACTTACATAGTTC
AGCCAGACCCTCCTTTGGAGCTGGCTGTGGAAGTAAAACAGCCAGAAGACAGAAAACCCTACCTGTGGATTAAATGGTCTCCACCTACCCTGAT
TGACTTAAAAACTGGTTGGTTCACGCTCCTGTATGAAATTCGATTAAAACCCGAGAAAGCAGCTGAGTGGGAGATCCATTTTGCTGGGCAGCAA
ACAGAGTTTAAGATTCTCAGCCTACATCCAGGACAGAAATACCTTGTCCAGGTTCGCTGCAAACCAGACCATGGATACTGGAGTGCATGGAGTC
CAGCGACCTTCATTCAGATACCTAGTGACTTCACCATGAATGATACAACCGTGTGGATCTCTGTGGCTGTCCTTTCTGCTGTCATCTGTTTGAT
TATTGTCTGGGCAGTGGCTTTGAAGGGCTATAGCATGGTGACCTGCATCTTTCCGCCAGTTCCTGGGCCAAAAATAAAAGGATTTGATGCTCAT
CTGTTGGAGAAGGGCAAGTCTGAAGAACTACTGAGTGCCTTGGGATGCCAAGACTTTCCTCCCACTTCTGACTATGAGGACTTGCTGGTGGAGT
ATTTAGAAGTAGATGATAGTGAGGACCAGCATCTAATGTCAGTCCATTCAAAAGAACACCCAAGTCAAGGTATGAAACCCACATACCTGGATCC
TGACACTGACTCAGGCCGGGGGAGCTGTGACAGCCCTTCCCTTTTGTCTGAAAAGTGTGAGGAACCCCAGGCCAATCCCTCCACATTCTATGAT
CCTGAGGTCATTGAGAAGCCAGAGAATCCTGAAACAACCCACACCTGGGACCCCCAGTGCATAAGCATGGAAGGCAAAATCCCCTATTTTCATG
CTGGTGGATCCAAATGTTCAACATGGCCCTTACCACAGCCCAGCCAGCACAACCCCAGATCCTCTTACCACAATATTACTGATGTGTGTGAGCT
GGCTGTGGGCCCTGCAGGTGCACCGGCCACTCTGTTGAATGAAGCAGGTAAAGATGCTTTAAAATCCTCTCAAACCATTAAGTCTAGAGAAGAG
GGAAAGGCAACCCAGCAGAGGGAGGTAGAAAGCTTCCATTCTGAGACTGACCAGGATACGCCCTGGCTGCTGCCCCAGGAGAAAACCCCCTTTG
GCTCCGCTAAACCCTTGGATTATGTGGAGATTCACAAGGTCAACAAAGATGGTGCATTATCATTGCTACCAAAACAGAGAGAGAACAGCGGCAA
GCCCAAGAAGCCCGGGACTCCTGAGAACAATAAGGAGTATGCCAAGGTGTCCGGGGTCATGGATAACAACATCCTGGTGTTGGTGCCAGATCCA
CATGCTAAAAACGTGGCTTGCTTTGAAGAATCAGCCAAAGAGGCCCCACCATCACTTGAACAGAATCAAGCTGAGAAAGCCCTGGCCAACTTCA
CTGCAACATCAAGCAAGTGCAGGCTCCAGCTGGGTGGTTTGGATTACCTGGATCCCGCATGTTTTACACACTCCTTTCACTGA

TABLE 31

MOUSE NOMENCLATURE
ICSGNM Top1
Celera mCG15357
HUMAN NOMENCLATURE
MGNC TOP1
Celera hCG38802
MOUSE SEQUENCE—GENOMIC
AGCCAGTCTTGTAAGCCTTTGTTCTTTCTTGGGGCTATGGCCGCTCGGCACTCCTTTGTGGCTTGCTCATAGATGAGCTGTTTTATCAGAGGCG
CAGCTTGCTCTGACTCTCCAAAAATACGCTCTGCTGCCTCTGTCATTTTGGCCACAAAATCTGAGAAGGATTCCTGAGGTCCCTGAACAACTTT
TGTTAATTGACCAGTGGTTTCACCTGCTCGGGAGAGCGCCTTTCAGGCCCTAATAGCCGTGGAAGAAATCTGGGCATAAGCTCCCCAATGGTAG
TTTGTCTGATCAGCAGAATAAGCTCCCTGACCCGTTAACAAATCAAAAGTCCAATCTCTCTGCTCTGGAGTCAAAGCAGCTGCGTTTGCTCGGG
CCTGCGCTTGTGCAGCTTGTGCCAAAGCGCTCTCTATTTCATATATTTGCCCATACTAGGGAGAGCGGCTTTTACAANNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNTGGTTCCGTATTTACGGACCGACTCGGCAATTTCTTTAATTTGTAAGTATTCTACCGGAAGCGTGGACACGCCCACCCTCGGCTCCTT
CAAAGACTGGAAATGCCTGTTGTATTTTCCTTTGTTCCTCTCTGGGAATGAATGAGTCTGCGCACTGCCTCTCTGCGCACTGCCTCTCTGCGCA
CTGCCTCTCTGCGCATCTCTCTGCGCATTGCCTCTCTGCGCATTGCTGACGCACTACGCAGGGCGGGGGCTCCGCATAGGGCGGACCTTGAAGC
CGACTGCCCTGAGGCCAATCAGCAAACTGGCCTTCGCCAGCCGCTTTTGGCTTCTTTAACTGATTAGCTAGCACTTTACCTGGCTGGTACCCTT
TTTTCTCATAATGAGCTGCTTTTTCCTCCCAGTCTGTTTCCTCAGAGGAGAATTCTTCATCTGCTTCAGAGCTACCAAGAGCTGGCTCCCCGAG
CCCATCTAGCGATGAGCACAGGCTCCTTTTCCTAGAGACCTCCGCTAATTGATCTTTCTTTTTTTCCCTTTTTCCTCTTTTTCTTCTAATCTCT
CTCCAGGTATTCCTACCTAACCTTAACTTTTTCTCGGGTTTAAGACCCTTGGAAAGGCCTGTATACTTATTTTGTGTACCATATTTCCTTTTTG
TTCCTACTCTCTCTCCCCACTTTACTTCTGATAGCTTGTCCTGAATTTCCTCTAGAATTTTTAGCCCTATCTTAATCACTTGATAACATGTGAA
AAGGAACAAAAGGGCTCCTAACACCAGAAAAAATTCAAGGCCAAACATATTTCACTTTACTTCTGATAGACTGTCTTGAATTTCCTTAGAAAGT
TTAAGATCAGACTTACCTCGTAAAGCTGTACTCACTGGTACTCTCGTTCCCCAGCTGAAAAGTTCTGAATTCATACAGTTGAATCCTTCTTAAC
AGTCTGCTTTACGGGAACCTTTATTACCGCGACCCGCAGTTCTGGTTCTGGAATGAGGGATCTTCCTTGCGCCAGTCCCGAGTTTTTTCTCGTC
CCGGGTTTCGGCACCAATTGTTATTAAACGCGTTCTCACGACCGGCCAGGAAGAACACCACAGACCAGAATCTTCTGCGGCAAAACTTTATTGC
TTACATCTTCAGGAGCAAGAGTGTAAGAAGCAAGAGAGAGAGAAAACGAAACCCCGTCCCTTTTTTAGGAGAGTTATATTTCGCCTAGGACGTG
TCACTCCCTGATTGGCTGCAGCCCATCGGCCGAGTTGACGTCACGGGGAAGGCAGAGCACATGGAGTGGAGAACCACCCTCGGCATATGCGCAG
ATTATTTTGTTTACCACTTAGAACACAGCTGTCAGCGCCATCTTGTAACGGCGAATGTGGGCGCGGCTCCCAACAACTTATATGGTATTCTCTC
TCTCTCTTTCTTCCCCAAGGGAGTGTGAGGCTTCTCTTCCTATCACCAGCCTTTCTCTTAAGCCAGTACCTCAGCTGGGTTTGTGTGTCCTCAC
CCACCCCCACCCCCTTTTTTTTTCCAAGACAGGGCTTCTCTGTGTAGCCCTGGCTGTTCTGGAATTCCCTCTGTAGCCCAGGGTGTCCTTGAAC
ACGGAGATCTCCCTGCCTCTGCCTTCTGAGTGCTGGAATTAAAGGTGTGTGCCACCACTACTGGGCTTTTTTTTTTTTTTTTTTTTTTGAGACA
GGGTTTCCTTTATGTACCCCTGGATGTCCTGGAACTTGCTTTGTAGATCAGGCTGGCCTCCAACTCAGAGATCTTGGTAAACTCCACAGAGGGA
GTAGCTACAAATGGGGTGTGAAGAAGAGGACCAAGGATCAACTCAAGAGGATCTAAGGGTTTGAGACTCACAACTGGGCAAGGTCAACAGAAGA
AATAAGGAAGCTCAGGGAGAAAGCAGACCTCAGGACCTGAGGACTGTGATATTATCAAGTAGACCCGAGAGAGAGAGGCTGTCTGTGGGAGTCA
ACTTAGGAACTATAAAGCTACCAAGAGGGAAATCCAGAGAAGACATGCTGTTGATAAGCTGGATGGGTAACCCAGCTCGTTGGATGTTGCCTCA
GAAGTATGAGGACCCGAATCCCCAGAGCCCACGTAGAAGCTGGACGTGGCGGAACACGGAATGCCAGAGCCTCCTCTGCTTCCTGTCTGGCCAT
CCCCTTTCCAGATATCTCACATATGGAGAAGGGCACAGTGACCTAAAGACAGCTTGGGAGATCAGGAAGGTAGTCAGCGTCTGGGCATAAGGGC
TGGCTTAACTTACTTAGCCATCCTTGCCCATAAAAAGAAGGGAGCACTAGAAACCATTCCCAGTGATGAAAAAGACGAAACAAGACAAACTCGC
AGAAGCACATAGCATAGGAAAGAGCTCAAAGTAAGTAGGTAGTTTTGGGGTTTGTTTTATTTGTTGTTAAGACAGAGGTTCACTCCAGTCAGTG
GTGGCGCACGCCTTTAATCCCAGCACTTGGGAGGCAGAGGCAGACAGATTTCTGAGTTCGAGGTCAGCCTGGTCTACAGAGTGAGTTCCAGGAC
AGCCAGGGCTACACAGAGAAACCCTATCTTGAAAAACAACAACAAAAAAAGACAGAGGTTCACTATGTAGCCTTGACTGACCTAGCATTCACTA
TGTAGACCAGGCTGATCTAGAATTAACAGAGATCCTCCTGCGTCTACCTGCCATATCCTGAGATTAAAGTCCTGATGCCTGGTTTCTAAAAACG
TATTTTTAAAGAAGTCAGGAGGGCACCAGGGACAGGAGAGCAAGTGACGCTCCTGCTACAGGGACTTCTGTAGGAAAAGATGTCCCTTGTAATA
ACCAAGCTCCTGGCAGGGACAGAGGAAGGGTAGTAGAAGACTCAGGGGTTTGGAGCAGGGAAACGGGAAAGGTGGTGCTTTCAAAATCTGACCC
TGGCTTAGCGCTATCACCATCTGGTTCAGTGAGGCTCTGGCGCTCTCTCTCTCTCTCTCTCTCTCTCTCTCTCTCTCTCTCTCTCTCTCTCTCT
CCCTCTCTTCCCTCCTTCGTTCCTGAGGAGAAACGCTGAGTCAGCGCAGGGCTTCTCTAGCTCCCAGAATTTTGGGGTGTTTTTCCAGAATCCA
GGGGGCTTGCCCCTCTGCCTTGCGCTGCCTGCTGCTTCTCTGGCCAGAAGCAGTTAGACCCAAACAGCAGCGGGATAATGACCTCCTGACAGCA
TACCCTTTGAGGAGAGTCAGCAAAGGCTGCTGTCACACCCAACCACTCTGTTCCAATCTAGATGGGTCAAAGTCCTCTGTCTCTTTTATAACTC
ACATCTCCATCCCCATTCCAACTAGCCTGCTATGGTCAGTATGTCTCCCAGCTAGCCTCCCCTTCTCCATTGTCAGCTCCCCGGGACTGTCCGC
TGTCCATTCTCTGGTACTCAATGGTTTTCAGATATCATTTAAACCTAGCTCACCTTGCTGTAGCTCCACGGTTATGGGAAGGGCCTTCCCACTT
TCCCAGCCTCAATCTATGCCACAGCCTTACACTGACTTCATCTGTCATCATGATCTAAAGTCATTTATGCCCATAGCTCTCTGCCTTGGCCATT
TGCCAGCTTTTCTTGTACTATCTTCTCTTCCTCAGATATTCCATTCCTCATCTCTCAAAAAATGACACGGAACTGGAAGGTTCCCCCCACCCCC
ATATTGTCCCTCCAAAATTTCCTAATGAGTTGGATGTGCCTCCATTGCAGAGTTTCCATAGTAATTGGTAGTTTGTCACCTCTCCCCTTATTGT
TCCTTGTATCTTAGGGTAACAGTTGAGGAATGTGACTTTCTCATTATTAGACTGAGAGTTTTTCCAAAATGAAGATCAGATCTTGTTTGTATCT
GTATTTACACAGCTTAGCATAGGACCTATCTACCTCATACTATCAACTAGTCTCACATTCCTTGGGGAGTAATGGTGGAATGAGTAAATAATCT
TATCACAACAGGGGTGTCTTGTGGAAGGAAGACAACAGAGGGTTTGGCCTGTAATCTCAGTACTTTGGGTGGTTGAGGCAGGATGAGAAAGATA
ATGGAAACACACAAATAGACCCCCTCCCACCCCGCCCTCCCACCCCCCAACCCCCAGCCACACGGCAGGCTCCTAAATCTCAGCTACTCATTCT
GCTATTCAAGGCTTGGGTCTTTCCCACCGGCCTTTCCTTTTATTCAGACAATTTTCAACTTTTGCTGAGCAGCTGCTGTAGCAGCAAAAGGCAG
CAGGACATTTCTGGTTGCTGGGATCAACAGCATGAACGTTTTCCATGGATCACCAAGCAGGTTCTATTTTGAGAGTGTCCAACTCTTAACAGGG
CATTAGATCTCTAGGCAAGGCATTATGGCAAGGGGAAAACTGCCAGAAAACTGTAGTCAGGAAAGCAGTGAAGCCAAGACTAGGAGACCACTAG
GAGTGCATGCCAGTAGTAAATCCAGCACTTAGGAGATTCAGGGCAAATGATCCAGAGTTCAAGTTTGGCCTCAGCTATATAATGAGTTCAAGCC
CAACCTGGGCTTCATGAAACCCTATCTCAAAGTTACAAAGTAAAAGTTACAAAGTAGCCGGGTGTGGTGGCGCACGCCTTTAATCCCAGCACTC
AGGAGGCAGAGGCAGGCGGATTTCTGAGTTCGAGGCCAGCCTGGTCTACAAAGTGAGTTCCAGGACAGCCAGAGCCATACAGAGAAACCCTGTC
TCGAAAAAACAAACAAAAAACAAACTTACAAAGCTAGTAAAAACAAATATATTTTAAAAACTGTTAGGAGGGATATCTGGGTCCTGAACCATGA
AAATCCCAGAGTGGAACAAAAGTATAGCCAAGGCCTTGAGATTCCCTGGCTTGAAACCAGATGGAAGTTGAACATCAGTCACATGACCCAACAT
AGCTTTCAGAAGCAAAAGGAGCAATGTATTTAAGTGTACCACAAAGGTATCTCACATTGCAATGAGGCAGAATTCACAGACCTGAACTTCATTA
AGGTCTTCTCTCTGCCTGTTAGCACTCTCCTGCCCTCTCCCCTCAACCTCTCCCAGCTTCCCCTGGCCTTGACAAAATGCTGCTCCTTGACGTG
ACATAGTACAGATCTATTAGCAAGAAGAACAACCACGTTACTTTCTTCTCAATTCTAAAGCCTCTAGAAATTCCATCTGAGTGGTTCAGTGTAG
GCCACTTGTGACAACTGTCAAAGAGGAACCACAGCACATTTGTGGCTGTGCATATGGTCCCTCCAGAACCTTGTGAGTCTGGGGGAAAGTCCTA
GGAAAGCAGGAGTTGTTCAGGCATCATCACAAGTGTTTAATTCTGGAAGACAACAGTTAGACCAGACTTTAATAAAACCTGCCTGTAGCAGCCA
GTGTCCTGAGTCCATTCATTCCAAAGGAGCCCAAGCCTTAAGCAGCAGATAGGAGATGGGGGTGAGGAGAGGGGAAAGAGGAGAGAAGAAAGAA
GAGAGGGGGGGGAAAAGATTTTTCTAGGCTTAAAGATATTTTGGAGGGATGGTGCTGGTGCATGCCTTCAGTCCCAACACCGAGAAGGCAGAAG
GCAGAGGCAGAGGCAGAGGCAGAGGCAGAGGCAGAGGCAGAGGCAGAGGCAGAGGCAGAGGCAGAGGCAGAGGCAGAGGCAGAGGCAGAGGCAG
AGGCAGAGGCAGAGGCAGAGGCGCAGAGGCAGAGGCTCAGAGGCAGAGGCAGGCAAATCTCTGAGTCTGAGGATAGCCGGGTCCATAGACTAAC
TCAAAACAAATAAACAAACAAACGAATCTCCAAAAGAACCCAACCCCCCAAGGGAATAAAAAGAAAGAAGAGGAGGAGAAAAGAAGGAAAAAGA
GAGACAGACAGAGAGAGAGACACACACAGAGACAGAGACAGAGAGACAGAGAGATCCTGTTTAAGCAGGAGCTGGAGAGCTAGCTCAGTAGTTT
AGAGCATGGCTACTCTTCAAGAGAACCCAGGCTCAATTCCACCACAACTGTTTGTAATTCCAGTTCCAGGGGATCTGGCACCCCTCACACAGAT
ATGCAGGCAGGCAAAACACCAATGCATGCAAAACATAAGGAAATTTTTAAAAAGTATTTTGGAGACACTGCAGACTTAAGCACCATACAGGTAT
CAGCATATTGAAGATTCTAAACAGGACACCAAGAAAAGCTCTGTTTAACTTTAAATCATAATGATGTATGTATTTGTGTGTGGAGTGCTCCTTT
TTTGCGTAACATCTACAAATCATCCGCCTTTTACCAGTCATGCTCTGGAGACAGGCATACATCATCATCTAGTTACTTTATAAAGGCTGAGGTG
ATCCGTCAGTGAATAGTCTTGAATTAAAACAGTATTTTCCATACCTGATATATTTGCTTTTGACCTTCCTGGAGTATGTCTTATATTCCCTCTC
TGTACTATTCTTGGATTTTTTTAAAAAATAAAAGCAGTCTCATTATGTAACCCTGGTTAACCTGAAACTCACTATGCTAGGCTGGCCTCAAACT
CACAAAGATCCCTTTGTTTCTGCCTGTGGAGTAGTGAGTTAATTAAAGGTATGGGCTACCACACCCAGCTGAATATTTTTATTTAAAATTAATC
ACCCAACATCTTAAAAAAAAACCCTTGAACTTAAACAGTTATTACTGTGGGTTTTATGTGGTAGTTAAGTGTTTTCCGTCATACACATTGAAAT
AGATATAAAGCAGTAAAAAATGTAAAAGTCTGTCTGTGTGGGGCCAAAAGTCCTCTCAAGTGCTTATGACCATGTGTGGGATCATTGGTTGGAG
AAGAAAGAGTGGCCATGTGGACATCACTAGCCAAACATAGATAGAGGCGTTTAGTCTATGGTGGGATGTTGGGTGGCCATGATTGGGCCTAGGA
TACCACCTTTGATTCATCATCCTGGGGCCTGCATTCTGTTTCCATGACTACTAGCTGGTCTTCCTGGGAGGGACCTGGAAAGCAAATGACGATG
ATCCCAAGTCACCAGGGGCTGGAAGCTAAAGCAGAGGCACTATTTTTCTTGAAGGCTGAATTATTTCAGCCTAACTGCAGTAAATGGGTGGGAG
GCAGATGCTTTGCTTGGTGGCCTGGGATTGCTTTTGACATGGGAAAGCGGCTATTGAGGAGACACTAATGCAGGCAAAACTGGCTCTGGCATAC
AGCTGTGGAGAAAGAGTGCTGGAATCAGGCAGACCTAAACATCAAATGATACATATTATCTACAGGCCCTTAAACAAAGTCTTTTTGTTTGTTT
GGGATTGTCTATTAAACACAATTATCGCTGTTGTCCCGTTGTCCAGATGAAGAAACTGGGACTCAGAGTGGTTAAACAACTTGTTCAAGGTCAT
GGAAGAATAGGAAAATTCTGCTTAGGAAAGCAGAATTGAAAAATAGGCCAGTTGACTTCCAAGTGTGGACTCTTAGCCACTGCATTGTTGTGTT
TGCCTTTGTTGATTGGACTCTTCTGTCAGAGGACCTGTTGATTCCTGGGGGCTAAGTGCTGAACAAGATGGAGAGGTCCTAACCCTCACTGACC
TCGAATTATATAAAGGAGACAGAAAAGAAGTCACTAGCTCCCATATGGGTAACTTAACAACTGTGATAACTGTTATAAAGGAGAGAGGCACATG
GGTACAGCATGCGGAAAGGGGCGTGCAGCACTTCAAGCTTGGTTTTTCGGGACAGTTTCTCTAAGTAGCCCTGGTTGTCCTGGAACTCACTCTG
TAGATCAGGCTGGCCTCGAACTCAGAAATCCGCCTGCCTCTGCCTCTCAACTGTTGGGATTAAAGGCGTATGCCACCACTGCCCAGCTGGCTTG
AGTTTTGAAATATTCACAAGAATTAACCCTAAGTGTTTATCACTCAATGTTTATGAACATTTTCCGAATTTATTTATATGGGGGATTGAGCCAG
ACTGGAGATGAATTCAGCCTGGGATGTTCTTGCAAATCCAATTGCTAATGTCATTAATCCCTCAAGACAGTTTGATAGTGGTCGATGGTCCACA
CCCAGGACTGTGAGGAGTCAGGCAGACCTTATAGTATGGTGTTCCCTAAATAANNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNTTGGAATATTAGCCCTCTCAC
ATCAAACTAATTCCTGTCTCACAGTAGGCGCTTAATAAATGTTTATAAAAAGAACAAATGCAGTAGGGTACTTGTGACACAGGGCTACATGACT
GTAAACTGGAGCTAAACCTGACTGAAAAATTAAAATAATTTTTAAAAATGGGTGTGAAGTGCTGGGCGGTGGTGGCGCACGCCTTCAATCCCAG
CACGTGGGAGGCAGAGGCAGGAGGATTTCTGAGTTCGAGGCCAGCCTGGTCTACTGAGTGAGTTCCAGGACAGTCAGGGCTACACAGAGAAACC
CTGTCTCGAACACCCCCCCCCCTACAAAAAAAAAGAATAAATAATAAATAAATGGATGTGAAGTATTCCAGAAGTCTGGACAAAGGCTGTGAGG
AGACTCCAGCTAAATGCCTTACGGCTGCTGGTGAACTAAGATCGGGGCAGCCAAGGTACGTGCGGATCAAACTTGTCTGGCTCTAGAGATGCAG
AAGCTGGAGACACGGTCCCTGGCCAGCTTAGCTGGGTCCCGCCGCGGCCTTTAAAGGCCTACCACGTGGCCGCCCGGGTGTCCAAGGTCCAGAG
GCGGTGTCCACGCTGCGCCTGCGTGCTGCTAGAGCCGGTGTCCCGCCTCTGAGAACGCCAGTCCCGTAGCAAGGCAGAAAGCAGAGAGTGGGCG
GAGTTAAGTCCGTATGCTAATAAAGGGGCGGAACTTGCGAGGCAAAACCGGCTCTGTCTCCCGGACCTCTAAGAGACCCGCCGCAACCCTCAGC
CTCTTTCCACGAGCGCTGACGTCGACGACGACGTCCCATTTAAAAGCGGCCGCGCAGGCGCAGTGAGGGGAAATGCGAACTTAGGCTGTTACAC
AACTGCTGGGGTCTGTTCTCGCTGTCCGCCCGCCCGGCAGTCTGTCAGTCGCGTCGCTACCGCCGCTGCCTCCTCCACTGTCCCGGGAGTCTCG
GGCCGGTGGCTGCTGTCGCCGTCTGTTCGTGCTTCTCAGGAGCTTCGTCCGCCCGTTCAGGCCGTCCCTGCCGTCCGCCTCTCTTCCCCCGCGC
TGCCTCTGAGCCCGCAGCGTTCGCACGCCGGCCGACATGAGCGGGGACCATCTCCACAACGATTCCCAGGTGCGACCAGCCGGCCCCCGACTCG
TCCCCAGCTGCTCGGGCCCGGGCCGGCTCCGGCCCCGGCCAGGGCCCCGGTCCCAGCCCCAGCCGGCCGCGCCGGGTTGACAGGCCCGAGTCTG
CGATGAGGGGCGGCCGGTTGGAGTCGCTTGGATGGCCGGGTCCCGGGCGGAGCCGTCAAGTTGGAGTTTGGTGGGGGGATGGCAGCCCCGGGTA
GGCCATTCGCGGGCCTCTGGGTTCCGGGGCGCCGGGTGGGCCCGAGCCCCGGCCCCCGGCCGCGCGCTCTCGTCCGCCGCCCCGGGGCTTCATG
TGCCGGTTGTTTCATCTTCCCTCTTTTCTTTCCTCAGATCGAAGCGGATTTCCGATTGAATGGTGAGTGTGCCCCCCGCCCCGCCCCAAGTGTC
CCCACACGCCGCTGCCTGCCTCCCCCGCGCCGGGCCGGAGCCGGGCAGAGGACAGACATGGCGTCCCAGAGACTAAGTCCCGGCTCCTCGCTCA
CCGGCCCCATTGTTCCCATCGGGCCGCCTCTGGACCCCCTTTCCCGGGGTCTCAGCACCCCCAGATCACGGCCTTCTCCAGAGGGGGCACCGGA
GACCCCGCGTCGACCGTCGCTGGGTCTCGGGCGGCCTTTCTCCGCCGGGATTCTTCCCGGGAAAGTCGCCTTGTCGACGGTCGGGACTTAGTCT
CTGCGCCATTTTCTTTTTCTCTCTCCTCTCCTTTCTGTGCCTGTGTCTTTCTCTCCCTCCCTCAGCTCCTTCCTTGAGCTGTCCAGAAAGAGCT
TTCTGCAGAGCAGAGCCATATAAATCACAGACCTCACCAAAGAAGGAAGGCTTAGGGACGGAATTATCTTTTATTTTTTCTTAATAGAACTTTA
TTTTGGAAGTTACTTGCTTTCTGCTTCATGTTTGCTTGCTTTCTTCTTCTTTTTTAATTTTTGTGATTCCAGAACGAAAGATTCCCTTAGGCAG
CTAAGGGTTTTATGGGTTTTTTTTGTTTCTTTTGTTTTTTGTTTTTAGTCCTTTTATTTTTTAAATGGTGAGATGTTTATAATAGCAGTTACAG
TTCTTGAAACTAAAGCCATCTTTTGTGGAGTTCTTTGTATTTTGGGATTGTTTTCCCAAGTTGTGCTGTTTGCAAAGATAACTCCCTTTAAAAA
AGTTTTCAGGTTGTCTGTGTGTCATCTAGTGTGAGTGTTGGACTTGAGCATATATTCTTTAAAAAAAATTAAAAGTATTAGTGACCAGAACTTC
CTATTGTGGCAAGGTAGAGTTTTTTGTTTTTTGGGTTATTTTTGAGACACAGTTTATTGTGCTTGTTCCGAAAGGTTGCTGAATTGCTCATTTA
GACATGGTGTGCTGGCCTTTTCTTTGGGGTCATAGGGAGTTTTAAAAAAGATTTTATTTATTGAAGTAGAGTGGCCAGCCCTGACTCCTGTCTT
CTTCCTCATTCTTCTCAGGGGCTGCTATTTTTTTCAGGAATTAAGAGTTAACTGGCTTTCCTCACATCCTTAGGCTCTTGAGCCCTTTCCAGTC
ATGTTTTCAAAATATTTCCACTAAAGTTACCCAGGGAAGAATGTTGGTGGGTGTCTCCCACCACCCCAGTAAGAAGCTCACATTTAATGGATTT
TCCAGCAATTCCATTGTAGTATGCTGGAAATTCCCACAGATTCTTATGGGTCCGATTGTGTTCAGTTAGTTTGGGTTCTGGGGAACTGAGTGGG
AGAAGCCATTGAGTAGGTTTTCTGGTGCAGCAGTCCATGGTACTGGCCTGCCCAACTGAAAGCCTTATGTTTATATTTCCAAGTTGGTCATCCA
AAGAGAGATGACACTGAGTTAGAGACTCTGTCTAGGTAAAGCTTTTCTTGAGCACCTAATATATGCCAGGTACTGTGCTAGAATTGAACCTGTC
ACCAACCACATCTGTCTTTGTTCAACTCAGCCTATTGGGGGAGTCACATCAGTTAATTACAAGGTAGGCGTTGGAGTGTATGTGTACCCAAAAT
GCTTTGGGAGCATAAAGGGCTTAATTTTATCTGGGGGTGGGGAAATTTCTTGGAGGTGACTGAGCAGGGTGTTGTATAAGACTAGGTACTGAGA
ATTGTCAGGGGTTGGATTATTTCTTGGGAGTCCATCCTATCAGGCTTTTAAACGAGGGAGATGAGACTGTCAAGCATATAGATAAGCTTCTTTG
GAAAGGGGGTAAAGAAAGGATGTGGTAGTTGTGGATGTAAAATGGTTTTGAGCTCTCTGAAGGAAGGTAGGAATTTAATATAGAAAATTTATTT
GCTTTTTAGTTGGGCCTAGTATGTAATATATTTAGCAATGAAACAAAATTGAGTCCTTTTTCCTCTTAAGTTTAACTTCCATTGTTCCCATCTA
AATACAAAAGAGCTATTGAATCTATGCATGATTATGGGCTGGGTATGTTATCTTTGCATTTTTATAGAGTGAATTGAACTGCCTATGTTAACTT
TTAGGATGTATTTCCATTTTCCAACAATGATATTTAGTATTTATTGGGCACCTAACTGTTCAGGCTTGAATCTCATCTTACTCTTTGGAAAGGA
CCCTTAGGAGTTAATTACAGTTATCTCCATTTGAGATGAAACTGAGTGTACTCAGTGAAGCCATGAACCTAGCCTCTGTGCAAACCGGATGATG
CTGGTATGAAGAAATGAAGTTTCCTGTTTTGTATGGGGCAATTAAGAGGGAAAGGGAAAAATTGATAGCATGCCGGTTACTTTTTACCAAAATC
TTGAAGAAAAAAAGCAGTTTTGTGTACCTACTACCTTAAGGTTCTAATGTTGTAGTGATTGTCTTTGTGGTTATTGTCTGGATGTTAGTGTCAG
TAACTATTTGCTCTATCAAGCGTACACTGTTAGCTAGTGATTGATAAATATAAAAGACAATTTAAAAAATTAAAACTAGTTTTTAATACCCAGG
CTTCTTAATTGATCTTTATAGTGAGAGGATATTTACTTAAACATGGTTTTAGGTGAGTCTGAACAATCACATTCATGAAGTAAAACCACATATC
CCTTGGTTTGCTTCCTCAAATCTTCTAAGATAAAAAAGATTTTCTCTCCTAACTGGAGCGGTTTTGTTTTGTTTTGTTTTAATAGTACTTAACA
TTTACAGCCTTTTGTGGGAAATTTACAACATTCTGGGTCATACTCAACTGCTGTGAAATGTTATTTGTGTGGTAAATCTATATTTAAGATATAA
GCAATGAGCTAGTCACTTCAGGAGCACAGCCCATACTTTGTATTTCCCTTTCACCTGAGTGAAAGATTCCCTTTGTCAGTAGCACTAATCATAG
TGAGCTTGAATTATTATTATAGTAGTGCACAGGATGCCTAGCTGCACAGCTGTTAACATTAGTTGAAGGTGCTGGTAATCCTTTTCTGTTTCAA
GAACTCTAAGTTCCTGTCTGTTTTACTGGAATTATGGAGCTACACAACTTCATTATATTGTAATTTGACAATGTGTAGATTATGAGTAGGTCTA
ATGGATTTAATTTGAATGAAGGGAATTGTTTTGATTGTAATGGTTGTACTTCTGTGCTTGCTGTGCATGAGTTATGATAGGCATTGAGGAGGCT
CGTGAGGCTTTAAGCCTGAAAACTGGTGATCTCTCCAAATGAAATCTGGAAATCTTTAGATTTAGTCTGGACGACTAATAGAAGTATTAGATTC
CAGTGGAGTCTTTTTACCCTTTAGAGCCAGACTCCTTGAGGGACATTTTTGAAATTAGTAAACTGCTGATATCTTAGAGCATATCAGATCTCAC
AAATTTCAGCCTGTCTATTTCTAGTAAAACAAGAAAATCTAGGGTTTCCTAAAGAAATCAGAAAAAAAAAAAAAAAAACCCTCATGGTTTAAAA
GACAAATGGGATACTATTTCTAAGCCTTGAATCTATTTTTCTTTGGTAAGCAGATAGTGTGGGACCTGGAGTTGGCTCCCAAATCTCATGTCGA
TCAATAGCACAGGGGCAGGGAAGTAGATTCTTTTTCCTGTCAGCATAAAAATGATGGATTTTAGAGTTTGAAAGATACTGATTTATTCTGGGTA
TAGTGAAGTGTGATAGCTGTTTGTATGAGTGACAGGAATCTTTATAGTTAACCTGTTTTAAGGTTATACCAAAGTTAGAATTTATAAGATTTTT
TTTTTTGTTTTCAGTGTGCGTGGCATGGGAAAGTGAAAAAAGTCTGCTTTTTAAACTTGTAAATATGTTAGAAAATTGTATATTATAGCAGCAG
TTGTCTTATGTTGATTGTGACATGGAAAATAATTTGTGTCATTTTAGAAGACCATATTGGGGCATCAGTGTATAACATATACTATAGATCTTCA
ATAACTTTTATCTAATGATGCTGAGTAAAATTGGCTTTCTTCAAAGCCAAACCCAGTCATTTTCCCAGAAGTGTGTTGTGATTTTTCTCCTATG
AAACTATGATGGACTCTAATTTAGAAGTTCACTTTGAAATGAATTCCAAGTGTAATTACTTTGAATATAGGTATAGTTTACCTTGAGACTACAA
AATGCTGGCAATATTTGGTGTTAAACCCAGAAAGTTTATATTTATTTGTTTTAAAAGAAAGAAAAGGAAGGAAGGAAGGAAGGAGGGAAGAAGG
AGGGAAGAAAGAAAGAAGAAAAAAACGGCTTAAAGGAATGGCATTTAGAAGTGGGAGTCCTTGAATGAAATTCTTTCCTGTGGTTTTAAATTAC
ATGATTATGCCTTGATTACTTTTAATCTACTAACTTGACAAATCAAAGTCATTTGGGCAAACAATTGAGAATTCACCTTCTTAAGGTGTAGAGT
TTAGGATAGAAGTTCCAGACTTAGTCAATGGAGTACTGTGTTTAAGGGAAGATTTCAGATTCTATTTACCTTAAGTAATAACCAGGTGTGTTGT
TTCTCTTTTCATTCTTTGTAAGTCTGAAGTATGAGAAGCACCATTCTATTCTGTGGCTTCAGCTCTAGTTGAAATAGGAATTCTAAAACTGAAT
ACATTAGTTTGGAAGAATTAGCTTTTTTTCAAGCACCTTCTCTTCCTACCTCCTCTTGACTGTCTAGGTGCTGGCAGTGTGCTGGGCTGATAAG
TGTGGGCCTGGGAACTTTAGAAGGGAATGAAGAAAGAAGGGTGGAGGGAAGCACTGTGGCCTAGGCTTGATTAGGAAAGAAAACGTTTAAAGCA
CAACCCTATTTAGATATTATAGTATTTTGTTACTGTTAGAGAAAGCATCTTAGAGTTGTTTAACAAGTTGGCGTTGTTTAACATGGCATCTGCT
CAGTATGTTCTCAGACAGTAGTGTTACTGTTATTTGGGGTAGTAATTCTAGATTATAATAGCATTGACTTAGATTGCCCACATCAATGTGAGTT
AACTGTTACATATGTCTCAATTTTGGTAAGGAGGCAACTTTAGTTTTTATTTCTTTTTAGTAGGTACTCATTTTTAATTTTTTGCCCTGTTTTT
AGATACATCCACTTTTCTACTATTCTCACCTATATACCATCCCTGACACTTTCTGAATTCTTATATGCTTTTCTACCAGAATATTTTATTTTAT
TTTATTTTATTTTTTTGGTTTTTCAAGACAGGGTTTCTCTGTATAGCCCTGGCTGTCCTGGAACTCACTTTGTAGACCAGGCTGGCCTGGAACT
CAGAAATCCGCCTGCCTCTACCTCCCGAGTGCTAGGATTAAAGGCGTGCGCCACCATGCCTGGCCCTTCTACCAGAATATTAATCCATATTTTA
TTTCATTTTTGTTTTGTGCCACCCTGTCCTCCCACTTTTAATTAANNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNTCCTTTCCCTCTCCCTCCCTCCCCCTCCCCATATCCCTGGCTGCCCTGGATTTC
ATTATGTAGAATAAAGGTTTGGCTGATCTCAAACCTTAAACTCACAGAGAACTACTTGCCTCAACCTCCTGAGTGCTGAGATTAAAAGCATGGA
CCACCACTTTCTTGAGGCTTCCTATTTTTTCTAAGAGCCAAGAACTAGCCAACCCTTGTGGATTTCTGAGTTCGAGGCCAGCCTGGTCTACAAA
GTGAGTTCCAGGACAGCCAGGGCTACACAGAGAAACCCTGTAAACCTCCCCCTCAAGAAAAAAAAACCGCATGTTTTTTCAAAGACCCCAAAAC
ATTTTTAAGTTGCAAGACTGAAGACACCTGAGAATTATTTAATCCTTATTAATTCTGTATCTTTCTTAAAGTGGCCAACTCAGCCGTCTTGAAA
TAGGGAAATGAGTATCCACGTAAGCTCAGGCCTGTGTTCCTAGCTTCTTAGAAAGCCGAGGCAAGAGGATGATCAAATGAGTTCCATGTCAACC
TCGGCAATTTAGTGAGACCTTGTCCTAAAAATAAAAAGAGGGAGGCAGTGGTGCTTTCAGACAAGACTGCAGCTACCAAAGCCCAAAGCCCAGA
ATTTAGGACAGAGCACTTGCACCATGAATCAAACCATCAGCTTTGCTGTCCTGCTGGCAGTTTTGGTGGTGTGCTGAGGGGGAAAAGGTGCCTG
CTTGGTGATACAGAGCCTTTGTCTAGTATGTGTTCAGTCTGACACTGCACTGTGGGGTTGCCCAAGCTCATATAACACTTCTAGTCCAGCCACA
CCAACAAGCACATCGTCTTCTCAGCCAGTTTTACCAACAAGGATAGGGCACCTCTACATGCAAAACTCACCTCTAGCCAGTCTCCTGTGGTCTT
TAATCAGAATATCTCTGTGCTCAGAGACAGCTACATTGCTCAGATACAAGGATTTAAGCCTTCTGGCTCAGAATACCACATGGTGGTAGCAGCT
GTTCCTTCTTTCATTCTCCCAGATTTCATTTCTCTGCTCATTCACTCCCTTTATCCCCTCATGCTTTGGAGGGAAACACCCTGTTCCTCATAAG
GTACTACTGTACTACGACATGCCATAAACATTCCACTCACCCTACCACTTTGTTCCCTGTACACCATTGGCTGATCTTGTAATCTTCCTTCCAG
AGTTGCTTCTATTAAAAATGGCAGTCAGTACAACTCAAGAGTAGAGCACACACCTACAATGTGCAATGCTATAGGTTTGATCTCTAGTACTACA
AAAACAACCCCTAAATTAAAGCTTATATTAATAGTTGTATGAGAGGATAGGTATTTGTGAGTTGTGAAAAAGATCATGCAGTTAGAAACTTCCA
CAGTGGCTCATAAGTCTTGTTAATGCTAAATACTGAATTGTCTTGAATTTGTTATTTTTAAACTACCACTTAAAGGAAATAAAAGTCACTTAGG
AATGCTTCGCTAGTATCCATTAGACTAAACCTAATGAGTCTTAGAATAAATCTTATACCATTGATCAAGAAGATCATTATAATAGGCAAAGACT
ATATAGAATTAAGGTAGGTACTGGTACTAATGTATTTCCATATCAAATTGTTGAATAAGATGAATTATCTGGTCATAATCTTCTGGTTAAGCTC
TATAATCCTGACATTTATTTGTACAAAGCCTAGTTTTATTTTGTATAATTTGGCTGCTTAAGGATATATCCTCCCCCAACCTCAAAGCTCCAAG
GTTGGACAGTATTTGTGCTTCCTTAAGGTTGTGATATCAAATAATTCCTAATGAGGAAGGGGTTCTCTAGAGCATAGGCAATTTGGAAGTTACT
TTTTCTGTTACCCAGGCCTTAAGAGCTCAGTTGATGGAAGTGAAGGAGGACAAGCCTGCAGAAACAGTCTTACTCTAATTGAGAAGGCTCGAGA
AGGTACATGTTCAGGTTAGATTAGTCTACCTCCTGGGAAAGGAGAAGATTGCAATGAAGGGATATCCTTGTGAAAATGAGTCATACTACAGAGA
CTTATGTAGGCAAGAATAAAGCATTCTGGGCTGGAGAGATGGCTCAGCGGTTAAGAGCGCTGACTGCTCTTCCAAAGGTCCCGAGTTCAAATCA
CAGCAACCCCATGGTGGCTCACAACCATCCGTAACAAGATCTGATGCCCTCTTCTGGTGTGTCTGAAGACAACTACAGTGTACTTACATATAAT
AAATAGATCTTTAAAAAAAAAAAGAATAAAGCATTCTAACTTTAAGTATTGTCCTTAAAAGATAAGTTAGTACAAATAGTATTTGTCTTGCTCC
TGAAGTAGACTAATGCTTCAGTTTAATATACTCTGCAAACTCAGGTTTTGACTTTTAAGGGACATTGTTCAGAAGCAGTCTACTTAGTGGTAGC
CCACCCCTGTAGATTATGCTATGTGGGAAAGATGCTTCAGCTTCATGGGTGGGTCAGACAGAATTCTAGGTATGGAGTTAGGTAAGGCAAAGGG
ATTCTCAATTGTTATTATGCAGAGGAGGTCATATAGTAGAATGTTTTGGTCCCAAGTTTCTAGATTAGCAAAATCCAGTTCTGTATTGTCCTCT
ATTGTAGCTATAATATGATAATACCATATCAGGCTTTTAACAAGGTAGAAAATATACCCCTAATATAACTACCTTGATCAAGAGCCCTGAGGAG
GAAAGGACACAGGTATGGATTATCCCTTAGAACCATAGAGTAGGTTTTCTTGAGGTATCTTTAGATGATCAGCTGAGATGAGAGACCCTTCCTT
GGGGTCGTAGTTTGTCACAGTGATTTGGGGGGGGGGGGGTTTCCTAAGGGAATTTTTGGCTTCTTACCAAATTTTTGGCATCAGTTTGTCTACA
ACCACAGGTCTCCTTAATTTCTTCTCTTGTCATTACTCATATTCTCTATTTTCCCAGTGTGAGCAATTTTCTTGAGTTCTCTCACCTTTTGTCA
CATGGTATCTAATCCCAACTGTGACATCCCATTGTGCCACCTAATGCATCCCTTTTTCCATCCAAGTAGATGGCACTGTTTTCAAGAGGGCTCC
TTGGACCATCAAGGCCATCCAGTATAATGGGAGTCCCCAGGGCCATGACACCGCCAGGCTCCACAGTTGTGCTCCCTAAGTACACAAAGGCATC
TTTGGTGGTAGCAGAGCTCAAGTGCAGCAGCTGTACCTGCTGCTTTTTATTGTGGTAAAAAATAGTTATACTGAATATTGTGTCATGTCGGAGC
TGTAGCCTACCAGGCTGGCTCATGGCAGCTGGTAACTGGTACAGCAGATGCCCAGGAACAGCTACAATGTTGCAAGACACCAACATGAAAGGTA
GTGCTGCCTCCCTCCCCAACCCTACCTTCTGTAAATGGAATTCTGAGGGCTTTTCTTTGCCCTTTTTGCTCCCAATTAACAGCAGGGCATTTGT
ATGCCCACCATTGTAGTCCGTTTTAGCTTCTGAGTATAACACAGAGAACTAATAAAGTTATTAAACTGTAGGCACTATGCTGGCCATATAGTCA
TCTATTCTAACCCTCAATGGCTGCCTACTTCCCAGCCACGTGGTTCTTTACCTGCAATCTGAGTCTTCTTACCCTGGCTCGTGCTTTTCCATGT
GTGTCCTCTTGGCAACTCCCTGCTGATCTGCTTGCCACCAGCCAGTCGTCCTGTCCTTCTCCAGGTCTCTCTTCTTCCTCTTCTCTCATCTCTC
TCCCTCTCTTTTTGGGAACCCCTGACTCTGATCAGAAGCCCCACCTTATTCACTTCTGCTCAGCTATAGGCTGAATTAGATTTTATTAACCAAT
CTGAGGTGATAGAAAAGAGTTTTTACACATCTTTGAGACAGGAGATGCTCCAAATACCATGACAATGCCAACCTCCAGACTGCTGGCAGGTCCA
TCAGCATCTGAATACACTGTACAAAACTGTACAAAACCCCCTCCCCCAACAACCTTCGGTTTTGTTGAATTTAAAATACTCACATGTGACTGAT
TGCTTTCTTAGACAGTACAACTTTGAATGTAGATACAACCCAAGAGATTTAGGTTTATATTTTGCTTGAATAGATGTTTGGGGCTCTTGTAACT
TCTCCAGAAATCTGAAGACCAGTGTGTATAGTGTGTTAAAATAGAATGAATTTTCCACCTATATGTTTACATTACTTTATTTTAGATAATGTGC
TAATAGAACTTACCACTTAGAAGTTCAGATAGATCATGGTTTTACTTGACCACCTCCCACCCCCAGTGCCAAACAACAGAAAATTTAAAGTCCT
TTAGATAGAATAAGTTCTTATTTTATCTCAGTGTCCCTAAGAAGTAGTTAATGTCTATATTTGGCAAAGTAGGTTGGCTTAAATTTACACAATT
TCATGGATCAGTATTCTTTAAAATGATTAACTGTTTGCTTATAACTTCATTTGAAAGTCTAAATTTCGCTTGGGCGTGGTGGCGCACGCCTTTA
ATCCCAGCACTCAGGAGGCAGAGGCAGGCGGATTTCTGAGTTCGAGGCCAGCCTGGTCTACAGAGTGAGTTCCAGGACAGCCAGGGCTGCACAG
AGAAACCCTGTCTCGGAAAAAAAAAAAAGAAAGAAAAGAAAAAAAGGAAGTCTTAAGTTTCTAGCATGTTACCTATATCAAGTTTGCAGTTACA
TTAAGTTGCAGTTAATAATGTAAACACTACATATACATAAATTATAGAGAATCTGGTACATTTACTTTTACGTAAGAGCAGATTAGAACTATTC
TTTCCTGTATGGCCGTCAGGGTTGTTTTCTCACGTGCATGCCTATGTTGAGTATGTGCTACAGCTTATAAATAATTGCGTTCACAATTAGCTAA
GACATATTAATCTACTGAGACAAAATGATATAGCCTTGGCTGCTGAGACCACTGACAATAGATATTTAAGATCTTAACAACTTGAGTAAATTAT
GTAGAGACCAGAAATAGAACCAGTTTCCTCTCAGTTTAGCTTTGAATATTATCAGGTTTTTGGATAATAGAGTAGGAAATGCTTAGAGGTGAAA
TGAATAGCAGAAATGTAATCAATTACTATTACTCTAGCAATGTCTAAAAGGCTCTGTAATTTCACCTGTCACTATTTTCCACTTTAGAAATTTA
GGCTCAGATGTTAATTAGCTCACAGTTGTGTAATTTTTAACTAAACTCTAAAGCAAGGAGTTTAATCCACTGTTTGATTGATGCCACAGATTGA
GTTAAATGTGAAACAGAATGAGGTGAAGGGGTGCCTGAGTGACACCCCCAGGACATCATGGGAGTAGTTAAATATCCATCAGACTGTGGTTCTG
TTTGTGATGGATCTTAAGTCGATTCATAATGGCTTTTTAATGTTTTATCAGCATTTATGTCTGTGTGTCCCTGAACTGGTGTTACAGACAGGTG
TGAGCTGCCGTGTGGGTGCTAGAAATTGAACTGGGGTGGGTGTGGGTTGGGGAGTGGTGGTGGCTGTGGTTCCCTGAAAGAGCAGCCAGCACTC
TGCTGAGTCATCTCTTCAGCTTCAGCCCTATCTCTCCTTTTTTTTTTTTTTTTTTTTGGTTTTTTTTTTTTTTTTTGAGACAGGGTTTCTCTGT
GTAGCTCTGGCTGTCCCGGAACTCACTTTGTAGACCAGGCTGGCCTCGAACTCAGAAATCCGACTGCCTCTGCCTCCCAAGTGCTGGGATTAAA
GGCGTGCGCCACCACGCCCCGGGGCTCTTCCTTTTTTTAAAGACAGGTTCTTGCTATGTAGCTCTGTCGTGGAACTCACTCCAGGCGAAATAAT
AGCATTGCCTGCCTTTGCGGTTTTTAAAGGCTTGTACCACCACTCCTGGATTATTTGTTTATTTTATAGTACTGAGGATTGAATCTCCAGGGCT
TTACCCACATTTGCTATACAAGTACTCGGCTCCTGACCCACATCCCCAGCCCAGGATTTGCTTCAGAGTGAATTCTTTTTTTTTTTTTTGGCTT
TTCGAGACAGGGTTTTTCTTTATAGCCCTGGTTGTCCTGGAACTCACTCTGTAGACCAGGCTGGCCTGGAACTCAGAGATCCTCCTGACTCTGC
CTCCCGAGTGCTGGGATTAAAGGTGTGCGCCACCACACCCGGCTCAGAGTGAATTCTTTTTTCCTCTCCTTTGCAATTTCAGAATTCTTAAGAA
GAGATTTTAGTCAGGAACTCCTTGTGTCTTCTGACTTAGAAAGATTTATTAATTATGTTCTAGAATTTTGAATTCTCAATGAGAACTATTGTAA
AATAACTGCTTTTGTATAGATTTACTTAAATTAGCCACTGTTAAGATGGACCTAACACTGAGAGTAAACTAACACTTTTATTAAAGTATGAATT
GAGACAGTTGGAAAGAGGGAGTGGAATTTTTTCACTGTTCTTAACTTCCAATGACAATAAGCTTTCAGGCTTGTTAGTTCCTGGGGGGGAGATT
CATGGAGAGTCCTAGTACATGGGCACACTTGGATGTTGGGCTTTATCACCAGAGGCCAGTTTGTTATCTGTCCTAAACAAAAACAGAATTTTAA
AGCATTAGAAAGTACAAGTGCATAATATTAAGTATGTTGATTTTGTATAAACTCCAGCTTTGGGGATGTGGTTCTAAGACCAAGATGCAGGGTT
TCAGTAAGCTTTATAATAATGTCTTCTTTTTTCTTTTGTCTTTCTTTCTTCCTTTTTTTTTTTTGGTTTCTTTTTCGAGACAGGGCTTCTCTGT
GTAGCCATGGCTGTCCTGGAACTCACTCTGTAGACCAGGCTGGCCTCGAACTCAGAAATCCACCTGCCTCTGCCTCCCGAGTGCTGGGATTAAA
GTCGTGCGCCACCACACTCGGCTATGTTTTCTTTTAAAAAATTAAGTTAATTAGTCCATTCTGATAGATGGTTTATTGTTATGTTATTTTGTGG
TTCTGGAAAGCAAACCTAAGTCCTTGTACATGGGAGGCAAATCCTGTAGCACTGAGCTACATTCTAAGCCAGTGGTCCGCAGCCTTTCTAATGC
CCTGATCCTTTACTCAGGATGTGGTGACCCCGATCAAAACATTATTTTCATTGCTACTTTATAATTGTAATTTTGCTTTTTCAAATTTATAATT
TATTGTAAAATATCTGATATGTAACCCTTATAAAAGGATTGTTCAATCTCCGAAGGAGTCATGACCCACAGATTGAGAACCACTGCTAAAGCTC
TTGTTTTTATGATTTTATGATGAATTTTTTTGGAGAAAAGTTCTTGCTATGTAGCCCCAGCTAGCCTGGAATACATTATGTAGATGAGTGAAGC
TGGCCTCCAACTCACCGAGATCTCCTTAAAGGAGGATTAAAGGCATGTGCCACCAAGCACAGTGTATTGTCTTTTTTTGAGACAAGGCATTGTT
AAATATTATAGGCTGGCCTCCAGTGCCCAGTCAGTCTACTTGCCTCTGCCTCCTTAGATTACATTTAAGGGCTATCACAAGTAGTTCCAGTGTT
CATTTTGTGAACTGGTTGTATATTTGTATGTCTGAGAGGAATAGAGATTTTCTATGACAAAAGTTGTACTGTGTGTGTACCTTCTTGGGGAAGA
CAGCTTTGGATAGTATATATAACTCCTTCCTCCTACACTGTTTACACCTGTAAATACTATATTTGAAGGAGGAAAGTCATACTGTCATATAGCT
GTTATAATAAAATCTCAAAGTCTATCAAGAAAAGTTGGTGTAAAGAGTCTGGCTTCAAACATGGCTCAGAGCTCCCTTTGTACCAGAAAGTTCT
TTCTGCCTTTGTTTTTATGGAAGGAAGGAAAGGACTCTTTCTTATAGCTTGATTTGGTTACTTTAAAATCTTGGAGATTGTGAAACAGTTCTAT
AATTGTTAATGTTTGTTAATTCTTTAGTGTATCTTCTCCACGAGATGGAATTGTCTCCTAGGCAAATGCAGTACCATTGAGCCTGGGTCACTCC
CCCAGCCCGTTTCCTCTTAGAAAGTGTAAGTGTGTTTGGATTGTCTTTTGATTGCATTTAAAGAAAAATAGACTTGTCTATTTTGTTAGGTGGC
CACCTACTAAATAATGCAAACATGAATAATGCAAACAGCAGAAAAAAGAGACCCCCCCACCTCAAGGTAGAAGAGAACCAACTCCTGAAAGATA
TTCTCTCATTTCTAAAGACTTTCAAGCTTGTAGGGGTGATTCTCCTATCTATGCCACCAGTGTTGAGATTATAGGTGCCTGCCACCTCATCTGG
CATTTTATATGGGTTCTCTAGTTGTGGACTAGCATTATGACATGCTGAGCCATCTCCCTGGACCAGCCTGGAAATTCCTCAGACTTCTAAGTAC
AGGATTGTTAGAGAAGGCCAAAAAAAGTACAGGATTGTTAGAGAAGGCCAAAATTGTATTAGTTTGTCTTTGTCAGAATCTCTAAGTATAGGGG
AATAAAACCCCAAACATCTTTTATCCAATGTTGCCTTATCAGGCAAGGTACCTAGGGCATGGTTCCTGCTTTGAGTGTGTATATGTATGTATGT
ATATATGTGTGTGTGTGTGTGTGTGTGTGTGTGTGTGTGTATATATATATATATATATATATATATATATATATCCAAAGCTTTTACCTTGTAT
GCAATGACTAAGTTTAATTCAGTTCTATATAAATTAATAATGAAGATAATTAAATAATGATTGCATTTCCTAGGCAGTTGTCAGTATGTTTGTA
ATTTGTTAGAAATTAGCCAGGAATTCTGTTGAAACTATTCTGTTGATTAGCTTACCAAAACAAAACAAAACAAAAACAAAAAACCCAGTTTTTC
TGAATCCCAGCTTTTGTTTCCTCTAACAAAGAGGAACATTTTTACTAAGGACACAGATATCAAGTTTATAATGAGCAGAGATGTAAAGCTCCTG
AGACCTTTCCCATGCTGCCTTTTGAGTGGTGCTGCTGAGACAGGGTTTCCCTTTAGCTGTAGAGCAAATCTGTTTTATCTGTGTGTGGTAGAGT
TTAATTATGATTGTTCATATTCTGTCTCATTCTTTTGAAGGAGTAGGTTCTTTAGCTTCTGAGCAGTTAAAGGGGCTACAGGTATTCTCTGTCT
CATTCATCTTTTTTTTTTTTTTTTGGCTTTTCCAGACAGGGTTTCTCTGTAGCCCTGGCCCCTGGCTGTCCTGGAACTCTTTTGTAGACCAGGC
TGGGCCTTGAACTCAGAAATCTGCCTGCCTCTGCCTCCCGAGTGCTGGGATTAAAGGCGTGCGTCACCACGCCTGGTTTAAACTTTTTTTAAAA
AATATGTTCCCGATAAGAAATCAACTTCATTTNNNNNNNNNNNNNNNNNNNNCACTGCCAAAAGTACATTTTACTTAATTTTGATCAGTCATGA
GTGGATGAAATTTTCAGCAATGATAATAGTGGCTAGCTGCTGTTGCTCATTGTCCCTCCTCTCCTGCTCCTTGCTCTCTGACCCCCGACTACCC
TTCCCTCCCACACCCTCCAGCTCTCCCACCTCCAGCCCCCCAAAACAAGAACAGGGTTTCTCTGTAGCTCTGGCAACACTGGAGCTCCCTCTGT
AGATCGGTCTGGCCTAGAAGTAAGAGATCTTCCTGCCTCTGATTCCTGAGCTCTGAGATTAAAGGTATGCTTCACCACTGCACAGGTTGTTGCT
TATTTCTGGCTGTAGTTAGGCCATCTGCCAGAGTGCTTGATTCTTAATTTCCTCAGCTCCTAGTAAAAGTAGCCCAAACTGCTTTTTTCACCTT
AGCTCAATCTCTAAAACCCACTTCATAAAAGAATAGAAGTAATTTACAAGCACAAGTAAATGTAGGAAAATAGCAGTAGCAAGACATTTATAGC
ATTATTAATACAATTATTCTTTGAAGGATGGTAATATAGAAATTCGGTTATAATTCTTCAGAATAGTCTTAATTTCCTCATTGTTTTTCTTAAT
CCTTTTTTGTGTACTTAGTGTTAGTCTGCCTTTTGATTGGTTCCCTTTATCTGTCACTTGTTTTTCCTCTTAGTAAACTTGCTGTCCATTCTTC
CTTTTTCTAACTCTAGAGTCTAGAGGTAGATACCTTGAAGGTATGTTATGTCTTCAGGGTAAATTCCTAGGCACTGCTGGGTCAGAGGAGGGAT
TGAGCTAGTTATTTCATTAGACATTGTGGAATTCTCCTGCCTCAAGAATTATATTTTCCAACTAGGTATATCTTATTTAAAAAAATATAAAGCA
AAATGCATATTTAGTAAGGAATAGCTATTACCTTTGGTGAAATGTGTATATTTCCTTTTCATTAAAATGGCCACATGTTAAGAGTCTGCATGTT
GCTGGCTCTTGTTTTAATGTCCATTTGTCTTGAGTGTCTTGTGTAATGGCTATTTAAGTCCATATATTCATTTACATGATTGGATTATAAAGGA
CAGAGCCCTTGTCTGATCAGTCTTTAGTAAGGTGCTTTGAACTTGAGAAATATAATGTTTGTAAATTAAATAGCCCTTATAGTATAATCCTGCA
GCTGCTAATACAAGGATGACTTTTGTTGTGTGCATTAACCATTGAATCCTGTGTAATGCTAACTAGAAATTGCTCCTACAGTGAGAAAAAGATG
GAGGATCTTCATCATTTGGTAGTTTGTTTTCTGCTTTATTTTTAAATGTGTGTATATCTGTGCCCTTGGAATCCAGAGATGAAATTATAGGTAC
TTGTGAGTTCACCCAACATTAAGTGCTAGGAACTGAACTCAGGACCTGGAAATTTATTTACTTAAAAGCCCTTTTAATTTTAAGGAGGAATACT
TTGGATAATAATAACAAAAAGATGTTGATTTTTCTAGTGTTTTCTATTTATTCATTTACTTATGCGTTTTTCTGGAATAATTTTATTTAAATGA
CACCAAAAAATTTGTTGCACATTTCTGTCTTCAAAGACTTTAGCTAGCATTCTTCCTGTGGTTTCATCTAGAGTTTTCTTTTTTCTTTTTTATC
TTTCATATTTTATTTGCATTGATGTTTTGCCTGCATGTATGTATGTCTATGTGAGAGTGACAGATCTTGGAGTTACATTTATAGCTGCTATGTG
GGTGGTGGGATTTGAAACTGGGTCCTTCGGGAAGAGTCAGTATATTTAACTACTACTGAGCCATCTCTCCACCCACCATCCAGAGTTTTCAAAT
GTTAAGAATTGAAACAATTATTGTTTTGTATGTGAGATTGTGTATGGGTTCAAGCAGAATGTCTTAAGAGGCATAGGATATTTAGTGAATAAGA
TTTTATGGTTAGCCAGGTGTGGTGGCCCACGCCTTTAAACCCAGTCAGTGCTTGGGAGGCAGAGGCAGGTGGATTTCTGAGTTCCGAGGCCAGC
CTGGTCTACATAGTGAGTTCCAGGACAGCCAAGACTGTTACACAGAAATACCTGTTAGTGAGTTTCTTTAAAAACCCAGTTGTGTTATGTGGAT
TTTTATGGTGCTATAGTACACAGTGGTTGGCATGTTTCATTTCTTAGCTATCAAGTTTTTTACAGTTCTTTTTGCATTTGATAGATACTTCGAG
TGTCACAAATGTTTTTACAAATATTTTAACAATATCTACTTTTTGACAAATTCCAGGAGGTATACTTAATCTTTAAAGCCACTAATGTATTCTC
TATAAACAAGTATTTTCAGCATGCATGATAGAGTTCTGTGTGCTTTTACTGGGTTTTACCTTTTACTTACTGCCTCTTCCTTTGTACTGTCTAG
GTCAGGGGTCCCACTACTGCCTTATATACTTTAGACAAATAAAGTCTTTGCTTTCTGTTTTACTAGGGCCTCTGGTTCTTCCAGCTACTAGGAC
CTCAAGAATATCCCTTACCTTCCCTCCTCCTCTATACTCTTTTTCACAATAAGTTCTTAGAACTGGGTTCAACTTCTATTTTCTTGAAAAATTT
CCCATTGTTCTTAATCTTAATCTTTTGCTATTACTGTCTTCTGGTTTCTGGTTTTTTTTTTGTCTTTGTTTTTGTTTTTCTTTTTTGTTTTTTG
TTTTTCCTTTAAGTATGATTTTTGGTCTTTTACAAATTGAAGTAGCTGTCATCCTAGAGTAGAACATTCTGACCCAGGGTTAACTATTAAAATT
TATCTTTTGGCACCTACTGTGGTGGCAAAGGTCTGTTTTCCCAGTGTACTTAGGAGGTGGGGGCAGGGGGATCTTGAGTTCTATAGACAGCTTA
GCAAGCCCTTGTTTCAAAAACCCAAGGGCTGGGAATATTCAAAAGGAACCAGTTTCCAGTGTAGAACAGGACTTTGGATTTGACCCCCTAGCCC
CAATTCCAAACAAAATTGTTTTTTTTTTTCCTCAATAGATTCTCTCTTGAATTACACTTATTTTCTTCTTTTACATAATAAGGAAAAATAAAGA
ATGGAAAGGAAAATGCTTAGAGCTCATGTTCATTATCTAAATGTAAAAAGTTCCAAGGATACACATGTGTAACTGCAGTTTGATTTCCTGTGTA
CCCATCATAACTTTAATAGCTAGCAATCTTTTGCTAGTGTTTTATATATCTTTATAATTCTTGATTCTAACTCTAATTTACTCAGAAATATTTT
TTAAGTACTTTTAAAGATTTCTTTTTTCTTTTGAGACATGGGTTCTCTGTGTAGTCCTGGCTGTCCTTGAACTCAATCTGTAGACCCAGGCGGG
CCTCAAACTCTACCTGCTTCTGCCCTAAGTGCTGGGCCTAAAGGTGTGATCCACCACTGTTGGTAAAGGTTTTTTTTTTTTTTTTTTTTTTTTT
TTTTTTGGTTTTGGTTTTGGTTTTTCGAGATAGGGTTTCTCTGTGTGGCCCTGGCTGTCCTGGAATTCACTTTGTAGACCAGGCTGGCCTCAAA
ACTCAAAAATCTGCCTGCCTCTGCCTCCCCAGTGCTGGGATTAAAGGCGTGTGCCACCACACCCAGCAGGGGTAAAGGTTTTAATCTATCAGTT
TACCTACTAGTAGTATACAGGGCATCTATACAGTTAAGTTAGTAGATTATTAAGTTTTTTAAATGTTAAATGATTGTTTAACAACTTTCTTTGT
GGCAATGCTACTTGTATAGTGATTTAAATCTATTGTATCATTTTTCCTCAGATATCCAAGCCTGCCATGTAGAACTTAACTGTATTGATTTATA
GAGCTCAACAGACATTCATATCTCTTATTACACTTACATTGATTTGTATGTAGAGGTTGGTGGGCAGACAGGGTTATGGAACACATGTTCTGGT
CAGAAGACAATCCATGGAAGTCATTTTTCTGCTGTGTAGATTCCTGGGGATTGCACATAATTGTCATGTTTGGCATCAGGGACTTTTCCAGCCT
GAGCTGGAAAAGCCATAGAGATTTCAATTAAGAACTTTGTCTTCTTTGTTTTCTGCTTTGTTTTGTTTTTAATTTAAAATCTCTCACAGCATCT
GTAGCACAGTTTGAGGATATACTAGATATGTAATGACTTTGAGTTCATATTATTGGCCTAGATGTTTTGTCCATGGAATTATTTACCAGCAACC
CTAATTTTCTCTGAGATAGTTAATTTTTACATTTTTAGATAGCATTTGTTTTCAAAGGCTTGGGACCCGGTTTTAAATTCCATTTTGAAAATTT
ATGCATTTCAGGAAGATAATAATGTGAAATCTAATAGGAACTACTCTGAATTTGTATTTTAAAGTTTAGTGCTTTTTAGTGACTTTTGCATGTT
TGTATTTGACAAAAATAAATTTGTTTGTTTATTTAGACAGGGTCTCTCTGTAACCCTGGTTGTCCTGGAACTCTCTGTGTAGACCAGGCTGGCC
TTGAACTCAATCTGTCTGCCTTTGTCTCCTGAGTGCTGGGATTAAAGGCGTGTACTACCAGGTCTGGCTTCAAATTTTAAAGTTTGCTAATCTC
TAACTTTATTATAAAAGTCGTTCATTATGGAAAATAAGATGAAGCACACTTAAGAACTCAAGTTCACAAATTTCTAAATTTTAAAATTACTACA
TTCTTTTTAAAATGTATGTCTTCCAGTCTTTTTTGTATGTGGTCATGGATTTATTGAATTACATACAGTGGTGCTCATTTTCTCTCCTTTTCAG
ATAATTTCCTTGTCATGCCTTTGAACCAAAGCAGTAGATCTTAACGTTCAATATTCTGAAGCCCATTTACTGATTTCTAACTATAAACAAAACA
ACACATGCTTTATTTCAGGTTGTAGTCCTGGTGCATGCATGCATTGTCATTTTTATGCCATGAGGTAAAAGTCACCAGTTTTATCCTGATTGAC
TCAGCATGTTAAGACTTTAGACTGGAGACTTAGAAGTCAGAGGGTCCTGTACACTGCATGGGAGATTTGAGTTCAGTCTTCTAACACTATATGC
TTTACTTACTATTCTCATCTCTGTGGGATGGAAAGTTCTTAGAACCAGGGCCTTGATCTTGCAAGTATTAGTGTTTCTCTTTCCTTGGGATCTG
TCTAGAATAGACATGCAGTTATGCCAAATAGCACTTCAGTTGCTTCAGATCAGCAATACTTAATTTATATATAATCTTATTGCTGTGAAACATG
CTACTTCAAATTTAGTTGATATAGGTTGGTTATAAGTGTCAAATACATTACATTCGTTAGTCTGGTTGAATGTAAGAAAGAATTCATTTAAAAA
AAAAAGTTTTTCGGGCTGGCGAGATGGCTCAGTGGTTAAGAGCGCCGATTGCTCTTCCGAAGGTCCCGAGTTCAAATCCCAGCAACCATATGGT
GGCTCACAACCATCTGTAACGAAATCTGATGCCCTCTTCTGGAGTATCTGAGGACAGCTACAGTGTACTTACATATAATAAATAAATAAATCTT
TAAAAAAAAATAATTAAAAAAAAAAAAAGTTTTTCATAAAAATCGCATGTCACCTAGAGTAACAGTCATCTGAAGGCTAAATTGGGCTGTGGCA
TATTCACTTCCAAGATAATTTCTTCACGTGGCTATTAGAAGGAAGTATCAGTTCCTCACCACATGTTTCTTGAGTGCTGCTAGTTTGGTAGCTG
ATTTCTCACTAGAACAAGGAGAGCAAATGGGAAGTTGCAGTGCCTTTGTGAGCTAATGTTGGGTGCTGTACTGCCATTTACTTGTGCAAGTGCA
AGAGACGGACGGTGCATGTGCATTTGCAGGTCAACATCAGATGTCTATCAGGCTTGCTCTGCCTTATTTTTAAAAGATGTATCTTATTACTTGT
CCATGTGTATGTATGAGTATAGATGGCCCTGGAGGCCAGGAGAGGGTGTTAGATCTCAAGGGGCTAGAATTACAGGAGGTTGTGAGTTGCCCCT
AACATGGATGCTATGAACTGACCCCTCTGTTATCTATCTCAGCAAGTACTCTTAACTACTGAAACTTTTCTCCAGCCTCTTTTCACCTTTTTTT
TTTTTTTTTTTTTTTTTTTTAGATAAAATATTTATTGAACTGGCGCTTCCAGAGATCTGTCTCTGTTTCCCCAGAACTGTGATATAGATAGGTA
CACAGCAATAAGCCAAAATCAGAGATACATATAGCACTGTTTCCCTCCTGTATTCTTAAAAAGGCACCACAGTAAATGAGAATAGAAAGGGAAT
CAGTGGGAGTGGGGATAAGTGTGAGTAATGGAGGGGATATTATCAAAGTACTTTATATGTTTAAATATTTTCATAAGGCTTAGGATGTAGCTTT
GTGGAACTGGGTGGGGGTGAGAATGGGGGCGAGCAACTGATGTTTTGGTAAGCAAAACTCAAATCAAGAGAAAATTGATTAAATAAAATTTTAC
TAAATTTTAAAAGAGGCTCTTTGAAAAACACCTTTAGGATTTATTTATTTATGAATGAATGACTGGTCATAGTGACTACAGGGATAACTTAGCA
GTTAGGAGCACTGGCTGTTCTTCCAGAGAATGGGGTGTTGATTTCCAACACCCACATGGAGGCTCACGACCATATACAGCTCCAGTTCTAGAGA
ATCTAATGTCCTCTTCTGGCTTCCCAGGCATTGCATACACATGGTACACAGACATCCATATAAGCAAAATACCCATATATATAAATCTCTCTCT
GTTTTTTTAAAAGCCTGTTCACTTTTATACTTTCTAACACTTGAGTCTTTGAGGTTGGAAGATATGTATCCAAAATAGCAAGCCATAGTATCAT
AATATATGTGGCCAAAAATAGCAATCCAGAAGTATATAAAGAACTTACAGAGGCAGGTGGATCTCTGTGAGTCCAAGGATAGCCTGGTCTAGAC
AGAGAAACCCTGTCCTGAAAAACCAGGGGGTAGGGGGAAGAACATATACAGCTCAGTAATACAATGTACAGTCAAACAAGCAAATACTGTAGTG
AACTTGCAATAGTTATTTAAAGGATGTGAATAGTCAATAAGCACAAATAGTTTTCATTAAATATTTAATCACTAGAAGAAAATTAGGAATTAAA
TTCATAATGAGGGTGCTGGCTGGTTAAGAGCACTTGCTCTTGTAGAGGACCTAGGTTTGGCTCTAACATTTTCAGTTCCAGAGAATCCAATACT
CTTATGGCCTCCACAGGGACCCAGCACACAGGGCATTTACATACATGCAGGCAAAACATTCATGGGTAAAATTTTTAAAAATAATTAAAAAAAC
ACAGCAGGCTAATTATATATATATATATATTATGTATATATAAATAAATAAAACATCCCCAACCAAAGCAAATTTTGCAAAAGAAAGCATTTAA
CTGGGGACTTGCTTACAGTTTCATCAGAGGTTTAGTCCGTTATCATGTTTAGGAGCATGGCAGCAGGAGGTAGGCATGGAGCTAGAGAGTAGCT
GAGAGCTATATTCCGATTCTGGCCTACTGTCCAAGAGGCTCCTGGGCCTGCTTTTGGCTTTTGAAACCTCAAAGCCCACTCCTAGTGATAAACT
TTCACTAATAAAGATACAAGTTTCAAACAGTGCCACTACGATACTATGGGGGTGGGGGTGGGGTCATTCTTACTCAAATCACCATAATATACCT
TTTAGAAGAGCAAAAATTAAGACAGTAAAGGCTGTTAGCCAGGCATTGTAATGTACTCAGGAAGCAGAGGTAGGTGGGGCTCTCAGATTGAGGC
CAGCCTGATGTACATAGAGAATTACATGCTCACCAGAGCTGCATAGCAAGAGTTTGCCTTAAAGAAAAACAAACAAACAAAGCCAACACTGGTA
ATTCTAGATGTTGCTAATGATTTGTAGTAGCTTGGAACTCTTAATAATATTCATGGAAATATATGAGAGTATGCCACTTAGGAATATATAGTTG
AGCAGTTTCCAATAAAGTTAAAAGTGAATTTTTTATATGATCTAGCAGTTTTACTTCATTTAGAAGCAAAGGTTCATCAACATGTGAGTTGTAA
ATTACAGTACTTCCATGTAATGGTTTTCACTCAGCAGGAAATGAAGCCATGGAGGCAGCTTAGTGGGACAGTGCTTGCTTATTATTTATAAGTT
ACTGGATTTGAGGCAAGGCAATGGGATGTCTTTGTGAAGTCTCCTTACCAGTAGGATGTGCTTTTATGGTCACTTTGCCCTTTGACTGACTTCT
GTTTCTAAAGTTGTTTGTTAAGGCAGGTAGGCAAATGATGCCTTATGATTCCTTGTGATTGACTTGTCTCTTATGATTGCAGATTCTCACAAAC
ACAAAGATAAACACAAAGATCGAGAACACCGGCATAAGGAGCACAAGAAGGATAAGGATAAGGACCGGGAAAAGTCTAAGCATAGCAACAGGTA
AGAATTGACCAACAGCGGGGAGGGTATAGGGAACTTTCGGGATAGCATTTGAAATGTAAATAAAGAAAATATCTAATAAAGATAAATAAATAAT
TAAAAAAGAATTGATCAATAGGGAGTTAGCATCCTTGGTGTCCATGGATAGGTATCAGCTTGAGGTGAGTAGCAAAGTAAGAGACTATATCTGT
TGTAGTTACTGAAGAGACTCCTTTAAGAATTAATGCCTCACTTGGTAAGTTTAGATCTAGATGTACAGAGAAGCACAGTGAGTCTTCTTTCAAT
ATGCATTTCTTTCTTTTTTTTTTTTTTTGGTTTTTCGAGACAGGGTTTCTCTGTGTAGTCCTGGCTGTCCTGAAACTCACTCTGTAGACCAGGC
TGGCCTCGAACTCAGAAATCCGCCTGCCTCTGCCTCCCGAGTGCTAGGATTAAAGGCGTGTGCCACCACGCCCAGCAATGCCATTGTTTCTTAA
AGCTATATTTATATTTAAAATTTATATTTTAAAACTGCTTTATTGGTTTGTCTTATTGTCAGCAGACAAGTTAAAGAGAAAATTCCTATCCTTT
TTTAATAATTGTGTTTTGTTTTACCATTTTTTGAGACTTGTTTTCTTTTTATATCCTTGGCTGTCCTGGAGCTAGCTCTGTAGATTTGAGTGCT
AGGATTAAAGGTGTGTGCCACCACCACCCAGCCAGTAATAAATTTTTTGAGTCATTAAAAAACTAGTTTAATTTTCTCTTAGGATATTTCTTCT
CAAATTATGTGCTCCTCCTGTTTTTTCCCATCACATAGTCATTTCTTACAACTTAGTTTATGTTTTATTCTTAATATTGTTTTTTCCCATCTCG
TAGTCATTTCTTACAACTTAGTTTATGTTTTATTCTTAATATTAATCTCAGCACTTGAGTCAAGTTTTGCTTTTTATGCACCCTTCTTCTCAAT
CTACATTTGTCTATCTAACACTACCAAAAGAGACTTGAGAGCAAAGAACACTGCCAACAATAAAGAAACCCATTTCATGATAATACAAAGGACA
TTAAAAATGTTTCTATAGTATCATCAGAGCCTCAAAATATGCACACACAGAAACTATTAGAATTTTGAAGAGAATTCATGATTCTAGTTAATGA
TTTTATTCTTTTGTTTTCAATAATTGTTATTCGTGCATGTAAGAAATTTCCAAGGCTAAGGGAGGGTTAAATAACAGAAAACAGTATCTGAGCA
TGGTTGACTAACACCTGTATGTAACCTTAGCATTCTGAGCCTGAGCACGGTGGATAACCAGAAGATTGAGGCCAGATCTAGGCTACATGGTGCA
AGACACTAACTTACAGAAACAAACAAACAAACAAACAAACAAAAAAGTGTAGTGTTGCACAACTTGACTTAATTGACATTTACAGAATAGTGAC
ACCAAAATCTTAGGACTGTGTTCAAGTAGGTGCTGAGATAACTCGTGCTTTTAGCTAGTCTCTCTTAAAATCATCCCTTTTACTTCTCCATGCT
TTTGTGTGTGTGTGTGTGGTGTGTGTGTGTGTGTGTGCGCGCGCGCGCGCGCGCGTGCATGCATGTTTTCATTTCCTTCTAATGTGCATAGTGC
TTATCTAGGAGGCTTTGGTTGCCTTCCTCTGCTGAATTTCCCTTGTCATTTGCCTTTACCTCACACTGTTAATTGAGCATTGTAAACTTCTTTT
AAATTTCTTAGATACGACATGTTTCTGATAAACCACTAGGCCTTAAAATATGCTATGGCTATTGTAGAGTACTCTTACTCTTCACTCCTTATTC
CCCTTCTCCTATTAGAGATTGAACGTAGGGCCTTAAATGCTAGTCAACAATACCACAATATCCCTGACCCTTTTTTAGATTGATAGAGATTGGG
TCCCACCACCTTGCCCAGGCTTGTTGGTTCTTGTTCCATAGGCCCAGGCAGGCCTGGAAACTACTTCATCTTCCCAAGCACTACTGTACCTGGA
CTTTCTTGTTGCTTTCTTAATATTCTTTTAGTAATAACTTTCTAACTATGTCATATTACTAATTTCTGTGGTCTCCTGAATATATTATTTCCAT
AGTAATTACCAGGTTTGGCTGTTAGGAATAATACTGCTATGAACATTGATATTTGAACACACATTTGAAGTTCTGTTCTCAATTCCTTCAGGTG
TGTACATCTAAGAGTGGAGGTATTGGACCATGTCAGTCGTTGCTTTCTTTTTCCTTTTGAGGTACTGGGGATTGGACTGAGGTGATTGAACATG
GCCAACATTCTACAGCTGAGCTACATCTGTAACCCTTAACCCGTTTTACCCTGCTTGTTATGATGGCCTAGCTTATGTATAATGCAATAGAAAA
ACAAAGAGGTTTCGATTTGTTGGGTTTTTTTTAACTTATTTTTTCCTCTTTACCATGAAGAAGCTTTATCATCAGTAGTGATCAGAGACATGTT
TTTACAGGTCTGATCAAGACATGCTGATTTTAATGTTATTAGGAATTTATTTTGAAAATATTTAAAAGAAAGATTAATATTTAAAACAAGCTTT
TCCAAAATGATTTCATGTTCTCTTACATATTTCACCAACCACAATTTATGATTATAATGAGCGAGAGGGACAGCATAAAGCAAAAGTGTATCTA
AGGACCTAGGGACTAGTTTCTTGTACTGTTTTACTGAAATCTTTTCTTAAAACTGGTATTTTATTGTCAGAACTATTACAATAATTCAATAGCC
TCTGTCTTGGGCGTCCTGGCCTGTCCACTAATGTGTTCTATGACTTTCACTAGGTCACCCAAAACCCTTTTTCTCCAGTGCTTCACAGGAGGGC
CCAGGAGTCAATAAATGTAAAGATCTTATGAAGTGCTCTACAAATGTGAAAATATATTTTGTCCATTTTTCATAATTACAGTTTTGAGTGAGTA
AGCACACACAGATTTAGGCAGCTTTTTGTTATGCTTTGCTTTTTGACAGTGTCTAAAAATGTCTTAAATGCCAGCTTATGTTACTTCCTTTTTC
TTTGGATTCCAAGTCTCTCAAACTTGTCACGAAAAGCACTAATCACATTAAATTCTAACTTTAGGAAAACTTCATCCCCGACTGTCTACTATTT
GGCATCTGACGTTGCATGGTTTACACTAATCAAATGAAATAATTACTTATATATTTTTAATTAGATTTATCTTATGGGTATTTTTTATGTATGT
GTACCTTCATGTATGTATGTGTACCACATGTATGCCTGGTACCCTCAAAGATCAGAAGAGGACATCAAGTACCCTGGAATTGGAGTCATGTGGA
TGGTTTTGAACCACTATGTGGATGCTAGGAACTGAACCCAGGCCCTCTGCAAGAGCAGCAAGTACTCAGCTTTGCCATCTCCATAGTCCTCATT
TTTTGATAATGTAGCTATAGTTATATGTTGGTGACTTACATGAATTTGGCATAAGTTTTAGTCATTTGGAAAGAGGGAACCTCAATTGGGAAAC
TGAAAAACTGCTTCCCTAAGATCGGCCTATAGGCCATTCGTGGTGGTACACACCTTTAATCCCAGTACTTGGGAGGCAGAGGCAGGTGGATTTC
TGAGTTCGAGGCCAGCCTGGTCTACAAAGTGAGTTCCAGGACAGCCAGAGCTACACAGAAAAACCCTGTCTCCCCCCCCCCCCCCCAAAAAAAA
AAGATCGGCCTATAGGCAAGAGGTGGGGGGGGGGGTTGTTCTAGATTAATGGTTGATGTTAGAAGGCTCAATCTACTGTGGCTGAGGTGGTAGT
TGTATAAGAAGGCTGGCTGGGCGACCCATGCAGAGTAAGCCAGTAAGCAGCATTCCTTTATGGCTTCATGGCTTCTGCCTTCAGTGCGATGTGA
CCCGGGACATGTAAGCCAAATAAACCATTTTCTCCCCAAGTTCCTTTTGATCAGTGTTTTATCAGAACAATAGAAAGTAGTAATATTTTACAGA
TCAGATATTTAAAAATACACTTTGAAATAAAAGGACTGTTTTTCTAGGTCTCAAGGCATTACTGGGCTTGTAGTGTGACCAGGTAGCTCTCTTA
ATCATTCTTAATCAGATTTTCTTTAATTTGTAATATTGAAAAGCACTATAAAACAAAAGGTCAAGTTCTGCCACTCACAAACTGTATCCTAATC
TAAGCATCTTACCTTTTTTGCCTCCTTTTCATTTTTGATTGATTTCCTGCTAAGTAATTTAGATAATGATGGTGATGATGACAATTATAACAAA
ACCTAATAAAAGATCATGTAGATAAATTGCAACTGTCAGTTTTTGTAGTGGAATCTAACTCAGTAGTTAATTATTCATAAATCTGATTTGGAAC
TAGGACTTTTTAGTTATTTCTAGCTTTATAGTTGAGTACTATCATTGCTTTTAAGATTTCATGGTGGAGGCAACCTTTTTGTTTGTTTTTGCAG
ATGTTGGTTATCAAACTTGTTGATTCTGGCTCTTTTTTTTTAAAAAAAAAATCTTTTTATTCATTTAATGTATATGAGTACACTGTTGCAATCT
TCAGACACACCAGAAGAGAGCATCAGATACTATTACAGATGGTTGTGAGCCATCATGTGATTGCTGGGATTTGAACTCAGGACCTCTGAAAGAG
CAGTCAGTGCTCTTAACTACTGAGCCATCTCTCCAGCCCCCAACCTTTTTGTTTTTTTAAGACCGGGTTTCTCTGTGTAGCTCTGGCTGTTCTA
AAACTAGTTCTATAGGCCAGGCTGACCAGCAACTCAGATACCCTCCTGTTTCTGCCTCTCAAGTGCTGGGATTGAAGACATATATCACCACACC
CAGCTCATGGAGACAACATATGAAGCTAGCCACTGAAAATAGTTACGATCACAGAAGGATGAAAGGATAATGGCTAAAAAAAGAGGTATCTGGT
GGGTACAGTGGTACTTGACTTGGGAGGTTATGGCAGAAGGGTGACCAAGTTCCAAGCCAAACTGAGCTATAACTGTCTCAAATATAATTAAAGG
AGCCTTTCAGATGATAAGAAAAGCATGAAGAGGTTAAGGAATGTAGCTCCGTGTTAGTGTGTACCCCGTAGAATATACAAGCCCTATGTTTTAT
TCCCAATATCACAAGAAACAAAACATACACAAGACATGTTACTGTTGTAGCATGCATTTTGTTTCAGAATGGATCCAATTAAGTGGATACTTTA
GTTACAAAGTGTAGGACAGAAATGAGGTCTAAAATGTTGTCTTGAGATAAACTACTGCTGAAAACTGTTTTAATTCAAGAAATTGCTTTAATTT
AAAGAAGAGCTTTAGTTTTTAAAGGGAGGAAGAAGCTACTGGAAACAGTTCAGAAAGGGGAATTTATAATACAGAAATGGTTCTTGTTTTGGAC
AATTGGGCAGCTTTTTATAATAAGATAGGTAGCCACAAAAGGCTGAGCTGTAAACCAGAATATAACGTATAAATTAGAACTTCGAGAAGGAATA
GAGGTTTAGAAAGTGCACTGGTTTGGGCATCTTTGGAAACAGCCTTTTTTTTTTTTTTTTTGGAAACATCCTTTTATCTTTTCCAGTCCGCAAA
CGAAGGAATAAGTTATTCTATTTGACTTTTCTGTAATTGCACACAATTTTCCTTCCTGGGCACAAGATGCAATGATTTTTTTTCAGACAGCAAG
TTCAAACTAACCTAATAGAAAATTCCTCGTGATAAACACTAAATGAGTTTTGTTTGTTTATTTGTCTTGAAATAGTTACCCAGGCTGGATTTAA
TTAAGATCTTATGGATTATACTTATGAATGTAAGTTTAAAATGTTATGACTTTCTGAGCACCCTTAGGAAATACAACCCTGGGACTTAATTCAT
CTCTCTGACTTCTATATTCAAAATACATACATTCTTATCCGTTTGTCAATCATAATTGAAAGTTGTGTTCAATTTACCTTTATTTAATGCCTTC
AGAGTGAGAGTGTGGTGATTCAGCAATTGGGATATACCAAAAAGCGGGCAGTAAGGTGTTTCTTGTAAGTGGAAAGCAAAAGTTCTCAAAGAAG
AGCGTGAATGGTTGTGTTGAACTCCAGAAAATGATGGCTGTGTATTAATTCCTTATACCCAATTTATAGGCCAAAATTTATAGTAGATATGTTT
GTATAAGGAAATCAACCCCCACCCGGACCTCCACCCCAACCCTGCCCTTGTATATAGAGTTGGGTACTATCTGGTGTCAGGAATTCACTACATC
CAAGGTGGGCAAGTGTCCTTGTGATTTTGCTTAGTAGCCTAATGATGATGGACATTCAGCAGCCGCTATGCTTTAGAGCCTTTTGAAAACTAGC
TGCTGTACTATTGTCTGGGAGACAACTTAAAGTTACCCGGAATTCTTTTATTTAAAGCATTTAAGCTTTATTTTGATGCCTGCTTTCCTTGTAA
ACACATTAATTTCTTTCCTTCTGTATTGTTAAAGCCTACTCATTCTCTTATAGGAGTGCCTGAGATGCCATTGTCTGTCACCCTGTCTGCTGCT
TTGCATAGTTGGCAAATATTAGTGGCTAAGTTCTCTTCTCAGAAGATGAGCAGATTCTACCTAATAGTGTTTGGTTTTTGTTTGCTTTGTTTGG
GAAGAATAGAATAGAATAGAAGTAATAGAATGGTTAATTTCATGACTGGAGACAAACTGCCTGAGTTGAACTCCTCATTTTCCCTGGACAAGTT
ACTTAAGCATTCTGTGCTTTTAATTTCCAGATGTGCAAAGTTTAGATACCAGTATTCATTTCAGGGTTTTGTTGTAGCAAGATGTGTATAATAT
TAGGTGGTAATATCGTTGCTATTTGCTAAATACTTTAATTGACCATAAAGTAAACTTTAAATTTTGATGGCTTCCCATAGCCAACATCTAGCCT
TCCTGAGTATCAGACTCTGAGTTTTGCAGGAGTACTACTACTCTCTATATTTTTTTTCTAAAGATTAATTACTTTTCTAGTGCAAAAGAGGAAT
GCTTTATTTTCCCATTTTACTGCCATCCCTCACTTGTCCTTTCTCATGCCTTCCTTCTGCTTAAGGTCTTTAAAGAAACCCTGCCATTTTTTTC
TTTAGTTTGTTTAGCTTCTATGAGAATCATAGACCAGAAGTTTCATAATTGTCACCTAAGAAATAGCATGGCTATTTTCTCCCACCATCTTTAT
CATTAGTTTCTTCTCCTCCTCCTCTTCCTCTAACTCTTCCTCCTCTTCCTCTTCTTCCTCCCCCCTCCCTTCTTCATGATCATCATCATTAAAA
TTCAGTTGTTTGAAGTATAAGTCAAGCATGCACATTATGAAAAAAAGGTTTTAAGAATTTTTATTATCCTTCTTTTAAAAATTATTCTTAGAGA
TTTTGAAGACATCATTATCTATCACCTCTTTGTCTTTCTTTCTGCGCTCTCTTCTGAAGTGACCCTGAGATTTTAAAGGAGGGGGGTGCGTGTG
TTGCGTGTATTCCATTTAGGGCTATGGATATTGTCTATTTACTTTCTTAAACATGACTTGCTTATTCAACCGGTGTGGTACAGTCTTACAGAGA
AACATGAGTATTGAATCAAGGAGTCAACCTCTGTTCAGAGCCATTCATTAGGATTTTTTTTTCTTTCAGAGTGCCTCATATCTGTGGTGTTAAT
TTTATATTTCATCCAAAAGTGTAACCATTACAGAATATCCATGTAGGTCCTGAGCACTATGTTTTTCAAGTTCCTTCCTATGCTTGTTCCTAAG
CATTTCTTTTATGTATTTAATTTCTAGAAGTCTTTTCATTGGTAAAGTTGGCCACTCTCTAGCCTCTGTTGTAGCCTTTGGTTGTTAAAACTAG
CTTTGTGACTTTGACATACTGTCTATTGCTGAAAAAGTGGCAAGATCTCCTGCCCAGGTGGTGCATGCCTTTAATCCCAGCCAGCACAAAGGAG
GCAGAAGTAGGTGGATGCCTGACTTTGATATCAGCCTGGTTTACATAGTGAGTTTCAGGACAGCAGAGTTATGGGGAGAAACCCTGTCTTAATA
CCCAACTGTAAATTATCTGAGTTTAACTAGTTCAAAATACAAACCTAGTTTATGATATTCCAAACAGATGGGTATAAAGGAGACTTAGTCTGTT
TAAAAAATAAGGTTGACATTTCATCCGTTTTACTGTTATTGGTACACAAGAGGATTAATACTCCAGGAGGATCTTTGAGTTTTCTACATAGATT
CTCATTAGGCTCCCTCTTCAGAGGAAGTAACAATTTGTGTGTTTTGTTTTATTACCCTAGAGTAGTCTTGATATACCATTACTACTTACTGATT
CTTTTAGTTTGGTGTTCTAATGAATAACAAAACTGAAATCTAGAGACTCAGGTTTGCATCCTCTGATTGCCAAAAGGTCATTTTTCCACTAAGG
CAGATTAGTTTTCTAATAATTTTCATTAAGAAAGAGAATTGAGAAGAGTAGTGTAATAACTAAAGGAAGACAGAGCCTGAAGCACTCTAATTAG
CCATTTATATTTTCATAAGAAGTAATCACAAAGGTTTGATCTTTTCAAGTATTTAGAAAATACCTACTTGGTGGCAAGATATGTCAAATCTTAG
ACTTAAGTACAACACCATTTATATCTGAGGTTGAAGAGGTGGTTCAGCAGTTTAGAGTACTTACTACTTTTCAAGAGTACCCCATTACAGTCAC
TTGCACCCACATGCCAGCTCACAGCTTACTCTAGTCCCAGGGGATCTGACACCCTCTGTCTTCCTCAAATACCAGGTGTGCATGTGGTACAGAC
ATACATGCAGGCAAAACACCCACATACAATAAACAAATATTTTTAAAAACGATTTAAGTGGAAATGTAATAACAATGTACAGGTTTTCTAAACT
TCTAAATGGAAATACTAAGTTTAATCCTAGCATTGTCCTCTATAACAGAATTTGCATTAGTTTTTTGTCTTGTTTTTTTGAGACAGGGTTTCTC
TGTATAGCCGTGGCTGTACTGGAACTCACTCTGTAGACCAGGCTGGCCTTGAACTTAGAAATCTGCCTCTGCCTCTGCCTCTGCCTCCTCTGCC
TCCTCTGCCTCATGCTGGGATTAAAGGCGTATGCCACCACTGCCTCCTTCCTTCCTTCCTTCCTTCCTTACTTCCTTCCTTCCTTCCTTCCTTT
CTTTCTTTCTGATTTATTTATTAATTTTATTTATGTGATGAGTACACTGTAACTGTCTTCAGACACACCGAAGAGAGCAGCATCAGATCTCATT
ACAGATGGTTGTGAGCCACCATGTGGTTGCTGGGAATTGAGCTCAGGACCTCTGGAAGAGCAGTCAGTGTGCATCAGTTTTTTAAGTGACCATT
TCTTCTGTATATACATTTGTCTAAAAGAAAATAAAACCTCTTAGAATTATTACAGAATTGAGTAAAATAAGGGTTGTAAGACCAAGTACAGCCA
GTCAGGTGTGGCTTACACCTTTAATCTCCCAACAGGGGCCAGCAGGTCCCTGTGAGTTTAAGAACAGCCTGGTCTACATAGTGAGTTATAGGCT
AGCCAGGCTGCATATTAAGACCCAATCTCAAAGACAAGCCAAAAACCCAAAGCACACACACCATGCACACATGCTACACACACTAGTCTGTACA
TAATACTTTCTCAACATCAAAAAACAAGTGCAATGCTGTAAGGATGTTGTAAGGAGAAGATGTGTTCATCATCAAAATGCCTGCGATTGTTTCT
GGAGTTTATTTTTATGTAAAATTTGTGTAGGAGACTAAGGGTTTTTGTTGTTGTTGTTGTTGTTATAGGTTTCTATAACTATTACTATAGTACC
TTTATAATAGAAGCTAAACATGGTAATATCAAAACCATGCATATTATCATGTGTATGATATATAGTGAGTCTAGTTTTAGTCTTTGCACTATAT
AAAGGATTTTTTTATACTGATAGAAACACATCATGAAAACACAATGAGTGGAATGATGTACTGTTGGTAAAATGTGGTCTCTGTGTGTGCCATT
AGCATTCTGTGTGCCATTCAGCCTTAATGAGTGGAAGGTTTGCTGTGGAAACTTGCCCTTTAATGTATAGCTCATATAAGTAACAGCTAACGAG
TGCTGGAGGAGAATGGAAATGAGGGAGTATGTAAACAATGCAAAGTGGGCCCTCCTTCCCTCCATCTGTAGATAGTGCTGGAATCCTCCTAAAC
CTGTAGTCCTGTATCTGCTAGTCTTTACCTTCTGGTTAATTTTCTAAGAATTTGTTTTTTTGAGGCAGGGTTTCTCTGTGTGGCCCTTGGCTGT
CCTAGAACTAACTCTGTAGACCCGGCTGGCCTCGAACTCACATTTGCCTGCCTCTGCCCACCAAGTGTTGGGATCAAAGGTGTGCACTACCACT
GCTCAGCTTCAGTTTGTGTTTATAACCCAGATGCCTGTCTTAGTTATGGCTACTTTTGTGCATAGAAGTGAGTGTAACAGTAGGCGATTTGATA
TGTATTCTGTTGCTTGGCTTAATTAAATGCCATTGCTTTTTGTCTTGTTTTTAATCTTCTTTATAGACAAACACCTTTTTTAGGAAAGTGTTTA
AAGGAAAATTCTTGGGTCTCTTGAGATCATTGCCAGTGAGGCCAGCTTTTTCTGCTCTGAGTCAAACAAGTGGCAAATGAGCTGTGACAAATCT
TATAGTCCTGACTGTTTCTATTGTTTGGGCTTACAGTTAGCAATTCTGCACCTGTGATGAACCCCTAATTCAGACCACTTGAGCTTGAGTTCTG
TTTTGCTTAGCTGTGGAATAATCCAGCAGGGAGCAGGTGCCTTAACACCAAAGAAAATGACTATCCACATTGTTCTGCAACATGTGGTTTTTGC
TGCTAGTCTCTTGTCTTTATCTACATTTACAGTCAAAGGTATTTTGTATAGTCTTGTTGGGATCTGTCAGTAGTATTCTGGATGTGTACTTAAA
ATAACAAATATTCCTTAATAGTTATCTTATTTCATATAACACAGTTTCCTCTCTGGTTAATCATTTAGATTGCATCCATTTTCTCATTTTCTTT
TTCCCTTTGGATACTATAGTAAGTACCAAAATAAACATCTTCCCTAAGAGACATTTCTAGAAGATCTTATATGAACATGTATGATCATTTTAAT
GACTCTTGCCTTGTGATGTAATATCTTGCTTTCCAAAATAGAAAAATGTACTTTTACGGGGCTACTAATATGACACATACCTCTTTCAAATAAG
ACTTAGAAATATGATGAACATAGACCTTGTCCTTGCTTTCAGGTGGGTCTTATTGTGAGGAAGAGGCCTGCAGGATCTACAAACTTCATCTGTT
TCTTCGTAGTTAGTGAGAAAGCTTTTAGTGTGTTGGGCCATTCAGTTTACAATGTTTTGTCCTGAGTCTGGCACAGTCAACAAATACTTTTTTG
GGTAATTTGGATTGAGTCCTCTTACAGCAGTTGTGACTGTTGGAGATAGACCTTTCAACTGTATATTTTATTGTTAGAAAGTTACAGAAATAAG
ACTAAACCTACTGTATATTCTACTACCTTTTAAAATGCTTGCAGAGCTTACATTATGTAGTGCTTTAGAGTTTATCCACCTGCTTCAGTAAGAC
CTAATCTGAAAGCTTAACTTGTCTTTTTTTTTTTTTTAAATCTTTCTTTTGAGATGCAGGGCAAAATCCTGGCTATACTTTCTCTAACACTGAG
CTTCTCACTTGTTCTCTGAGACAACTTCCCCAGTGCCCCCTTCTGCCATGTAGTTGCTTGTTTGCCAAGTAGCTATTTGCAGACCTCCATTGCT
GAACATGCCAGCCTAATTCAGCAGCATAGATTTTTTTTTTCTTGGTCTTCCTCTTTTCTCTTTCTTTTCCTCTGGGAGGTACTGAGATTTCCAC
CTGTGTGCCCTGAACTTCCTTCTGAATCTCTTAAGTTTTTTTGTTAATGAGAGTGGGAACTCCGAATTTCCATGTAATAATTAGTCAACTATGA
AGGCATCCTAAAGAAGCTCTTGTTAGGATAGGAAACAGGTGACCATGTTCAGGATTCTAGCAACTTTATTTTTAAGGGACTTGATTGATTGCTG
AATCAACAGACACATAAAGGGCCACCATCCCCCAGGTCTCATTTGTTCCATGATAACCTCATTATGGGTCATGAAGCAAGGTTATGCCATGGAA
TCGTTTATATTTCCAGTTGTTCATGTGTTCCTAACACAGCAGTGCAGTTATACACTGGGTACATGTCTTTGTCTCTCCTGGATCACCATGGGCT
CCTGTCCCAGCAGCAACTTGTACCTAGGAGCTGAAGGGAGATCAATACAAACTCCTTATGACTTTTAGTGCTTTCTGGTTACACTCGGTGAATT
TAACCCAGGAGGTCCCCTCTGATACAGCTAGCAGTCTTTGAACCTTTTTTCAGGACTGTGTAAGCATTATAAGTTTTCATGGTGTATGGCTTTT
GAGTTGATTTTGTTGTTCTTACTCTAGGCTAACTTTTTATGTCTTATTTTTAACAGTGAACATAAAGATTCTGAAAAGAAACACAAAGAGAAAG
AGAAAACCAAACACAAAGATGGCAGCTCAGAAAAACATAAAGACAAACATAAAGACAGAGACAAGGAAAGACGAAAGGAGGAAAAGGTACAGAA
CTTTGCCTTAAGGGGAAACAAGAGTTAAGATGTACTTTTATTTTGAATACTTTAGAAGTGGTAATGAAAGTAACTTTGTTATATCTACCCTTGA
CCCTGTAGCAGACTTCAAAATTAGCAGGTAGTGCTATAGCAAAAACTACAACTAAACACTGGCTTCCTTGCAATGTACTCATTAAACTCCTCAC
TCCCCAAACAAAAAAGATTTTAGATTCAATCTGTCTGAAGCTATGATAACCAAATTATTTAACACCATGTCTTTAACAATGCAATAAGTATGTT
TATTTTATACTACAGTGTGCCTATAATGTTTCTGGAAACATCTTTTCTGTTTTAGGGAAAGTTTTGTTTTTGGAAAACCTAAAGCTTTATTGGG
TCTCCTCTGATTGAAGATTCTGGGGCTGTGTCTATACTTAGTTGATAGACTGACTAAGTTTGACTAGCATGAAGGAAGTCTTATGATTTCCAGC
ACTACTACCAACAGTTGTGTTTGTGTGTGCCTGTTATTCTAGCACTTGGGAGGTGGGGAGATAGGGCGAGTAGAAACTTAAGGTCATACTTGGG
CTACAGAGTGAGTTCAAGGTGATCCCNNNNNNNNNNNNNNNNNNNNTGTGTATGTGTCAAGGTTTTTCAATGCATTGTTTTTTTGTTTTGTTTT
GTTTTTTTGTTTTTGTTTTTTAAAGTGAAAAAGAAATTTGTAGGTGGGCAATCATACACTAGAGAGGGCAGGAAATAGACTAGTCTATTTTATG
GTACCCTCACCCCACTGTGAGGCTCGCAGCAAATTTTTGAGTAGTGGATATATTTATTGGGGAGATGAAATCTACTGTTATTTTAAGATTAAAA
AGTTGCTGTTTTGAGGAAGAACTTTCTCGATTAATTTCACCAGAATTGTCAAAAAACAGCCCTGGGAAAATGTCTTTAGTTGACCCACTTCAGT
TTTGCTAACAAGAAGAGTCAGTCCAGGATCAAGATGCTCCAAAATAGCCTGGCATGATCTTGCCATTCCCGAACACTCAATGACTGTGGTTATC
AGCACAGGACACGAGCAGTATTGAGCCTGTCAGCATTCTGTCTTGGAGAAGGGTAGATGAGGTCTCTTCCTTGAAGACTACCATCCATTAATGG
TTATTAGAGGAAGTTGAGACATTTTCTCAGGTGGTATAGTCACTGTTAAGGTGCCTATCTACACTCCTATAAGCAATTCTGATTCAACTCAGTG
GGACATCCAAAAAGCAGGAAGAAAAGACAAGTCATGGCTGGGAAGGGGATCATTAGGAACGGGGAGTGAGTAGATCCGAGTACCTCATATACTT
GCATATACCATAATGAAACCTGCTTTATGTATAATTAGTGTATGCTAACAAATTTAAAATCTCCCAAATTGATGGTAATCTTCTTGAATAAGAG
TTTTTTGTCTTGTTTTAGTATTAAAAACTTTCAAAGTACTTACTGAGAGGCTGTGGTTTTACCTTGCAGATTAGAGCTGCTGGGGATGCAAAAA
TAAAGAAGGAAAAGGAAAATGGCTTCTCTAGGTAAGACCTTTTGCTACAGCAGCTTTTTTTGGGAGGGGGGTGTGTGACTCTGCATGGTGGTGT
CTAAGCTTTTCCAACATGAACTGAACTAGCCACCTTGACAATTCAAAAAAAGTCAGTATTAAGAGCACTTGACCTAGGCAAACTTTGGTTTTTT
AATAGTTCTAGTTAGTGAGACTGAACAGCATTTATCCAGAAATCTACCAGAAATGGAGAAGGCTATCAGAATGAGGTTTTTTTTTGGTTTTTCG
AGACAGGGTTTCTCTGGCTGTCCTGGAACTCACTTTGTAGACCAGGCTGGCCTCGAACTCAGAAATCCGCCTGCCTCTGCCTCCCGAGTGCTGG
GATTAAAGGTGTGTGCCACCACGCCCGGCCAGAATGAGATTTTAAAAGTTGAGGTATATACATACCACAAAATTTGCTATGCTAGCATATATAT
AGCTTATGGGCTTTTATGGTATTTACCTCACTATTTCCCAATATTTCAAGAGAAACTCCTTGGTAGTAATTATGGAATTTCTTAGTCTTAAATT
AATTCCTCCTTTGTAATATAAGACATACGTAAACCCTTATTGGACTTAATTTTTTAATATAGTGTATATTACCTATAGGCTGTTTTTACACTCT
GCAGTCCATCTGTTCTGTTTTCCTCATTTTCAGTTCCCTAAAGTAAGTGCTCAAGTCCACTCTAGATTGAGAAGACCATTTAAGATCACATTGT
ATATTCTGATGTCTTCCATGGAACCACGGAATGGATTTGTTAAGACTTGTTGAAAGCATTTGCCTTATTGACTGTAACAGAGTTTTAAGAGCTT
TGCTTTCTTGAGACTTTGTATCCAGGTCCTTTAGAAAAAGAGAAGCAGGTGCCTTTTATAGGGATATTTTACAAATGGTGTAAGAAAGCAGAAT
AATAAAGTTGAGTAAGCATTCTACAAAGTACCCTAGGAATTCTGTTAAACAATAAAATGAGGCTCACGATGTCTATTCTTTGCCAAGAGATCCT
GCAAGATATGAATAGATTATTTGAACTTTCGTCATGAATTCACTTAATGACTTGGGGGATTAGAAGGACAGGCGTGTGTGCTTTGCCCCCGATT
GTCGTTGCTGGCCTGCTTGTCAGCAGTGAGTCCTGTAGAGAATCCAGATGCTTCACTTGAACTTCATCTTCGAAGTGTGGTAACTCACTATTTG
CTTTCACACACGACACTTAGGATGGGGCATTAACTTTGAACCATTGGATGTTGTAGTGTCTTCTTTGGATCTCCAGGGCTTTGTAATTGAAGGC
GAAACAGCTGTAGAAGTGGCATTTAGGAATTGGGGAAAATGTGCTGGAATAAGAAGAGCTAATCTTTTTAAACCTTCAGATTTTGTTGCAAGCA
AACTGGTTTTAATGCTTTTAGAAAGAAAGGAGAATACATTATCTATACTTCATGCTGTGAATATATTTTACTGTTGAGACTAGATACTTACACT
TCAGATCCACCACTTCATGAATGGCAGCTTTATTTTGGGAGGCCATTTTCTTTACTGAGTTAAGTCAGTGAGGTCTAATTTGAAGAGATGTAAC
AAGCCAACCTAGGAAAGAACATTCCAAGGTTTCTGAGTGTTTGTCCGTGTCACCGGCAATTCTCCTTCCCCTTCCCCTTCCCCCATCATCATCA
TCACTACCACCACCTCCTCCTCCTCCACCACCACCACTACCACCATCACCATATCACTATAAGATAGCTGACATACACGCTCCCCTATCTCATT
ATTAGTAAATGGCAGAGTCTTCTGAAGTTCAGAGTGGCTTGAACCATTACATAAACCAAGACGAACCTCCACTCCTGCTCCTCCTCCTTAATTG
TTGGAAGTGTTGAAATTACAGACGTGTGTACCCAACTTAATTTAAAAAAGAAAAAATTAGATATTTAAATTTTACGTGTATGAGTAAATGTTTT
GCTCATATGTATCTGTGCTTGCCTATTTCCATGGAGGTAAAAATGAAGTGTTGGATATCCTGGAGCTAGTTATAGGTGATGGTGAGCTTCCTGG
TGAGTGTTGGATAAATCTGGGCTCCTTGCAAAAACAAGTGCTCTATAAGTGCTGAGCCATCTCTCCAGACCCCCAGTTTATTCTTTCTAGTTAG
TACAGGTTTCATAATGTCATTGTACTCTGGCTCATAAATTCTCTAGATAGATAGAGATTTTGCAAAGGAAAGAAAATGTGACATTCTTTTCTCC
CTTGCTATAAACCTATCCCGTTCTATTTTCATGAAAAGATTTTTTTTTTAAATGTATATGACTATTTTAGCCTCATGTAGTTCTATACACCATG
CCTGTGCCTAGTCACTAAAGAAGATCCCCTGGAACTTGGGTCTATGCTTTCTGGAAGATTAGTAAGTGCTCCTAAACATTATGCCATCTCTCCA
TCTCCTTTCATGCCACATTTTTAAAAATTGAGGGTTTTTTGCATAATAAAGAAAACATGTTATTTCTTCTATTTTTCCCTGCAAATAACACAAT
TTTATTCTTTATGCCTGAATAAAATTCCATTGTGTATATGCCATATTTTCTTTATCCATTCATCTATTGATTAGCATCTACACTGGTTCTGGAT
TTATACTATTATGGATTTACAGAGAGATCCATGTTTTATGGTGTACTGACAAAGCTTCTCTTGGGCATATACCTAGGAGTAGTATAGCTGGGTC
ATGTTGTGGAAGACCTCTATAGGATTTTCATAGTGACTGTACCAGTTTACATCCTCAGTTGCATTTTTTTTTCACATCACCTTTTATTACTGAA
GGAATTCAGGGCAGGAACCTGGAGCTAGGAGCTGGCGCAGAGGTCATGGAGGGGTGCTGCTTACTGGCTTGCCTGTCATGGCTTGCTCAACCTG
CTTTCTCAGTGCTCAGGACCAGCAGCCCAGGGGTGGTCCTACACACAATGGACTGGGTTCTCTCACATCAATCACTAATTAAAATGCCCTACAA
CTGTATCTTTAGGGAGGTATTTTCTCAGTTGAGGTCCCTTCCTTTCCAATAACTGTAGCTTGTGTCACTCAGGTGGACATTAAACTAGGCAGGG
CACCTACTTTATGTTATTTAACACAATTCTGACAATTTAGTAATCCTTAACTAGCTGCTTTGTTCTCTCTTTAGTCCGCCACGAATTAAAGATG
AACCTGAGGATGATGGCTATTTTGCTCCTCCAAAAGAGGATATTAAGCCATTGAAGAGGCTTCGAGATGAAGATGAGTATGTATTTCTTCCATT
TTGATTAAAGTGGAACATATAAAGAAATTTCAATTTTGAAATAATGTTTGTTGCTGCCTGATTAACAATGGTAAAATTGGAATCAACCTTGTTC
ATCAACAAATGAATGGCTAATGGACGTGTGATCTGTGGGTGGATAGTAGAATGTGCTGCTCATCTCTATAGAAACATGAAATTAATAGTTTTAT
AGGAATATGGATGGATTTAAAATATGTAATAGCAAGCAAGGTCATATAATCTCAAAGAAAAAAACTCACATTCTCTCCTGGAATTCTACCAATA
CTTTGAAGGAAGAGAATAGGAAAGGCTAAGTAATTGAAACAGATGAGGAAGTACTGAATGCATTATAAACGAGGATATAAAGCTAATTGTTTTT
CTACTTCTGTTTTTATGTGGAATCTTCTTTTTTTCCTCCCCCACCCTCTTTTTCCCCATTTGGTGGTAGATAAGGGCATAAACTACATTCAGTA
TTGAAAATGTAAAATTGAACAAAGCTGTAGAACTTCATTCTGAGTGCCTGTTTAAAATATAAATGTTCATTGAACTAGATCTGTTAGTTTTAGT
ATGGTAATTTGGAAGAGTTTATATATACATGTGTGTATATATATATATGTATATATAAATTTATAAAAAATTATAAAATTATAAGAAGAGATAC
TTCATAATTTTTATTTTATTTATTTTTTGTTTGTTTGGTTTTTTGTTGTTGTTTGTTTGTTTGTTTGTTTCGAGACAGGGTTTCTCTGTATAGC
TCTGGCTGCCCTCGGAACTCAGAAATCAGCCTGCCTCTGCCTCTCAAGTGCTGGGATTAAAGGCATGCAACACCACCACCGGACTACTTCAACA
ACGTTTAAAAAGAAAAGGCCAGAGCCAGGCATGATGCCACCGCCTTTAATCCTATTACTCAAGAGGCAGAGGCAGGGGATCTCTAAGTTTAAGG
CCAGCCTGATCTATAGAACAGGGTCTAGGACTGCCAGGGCTACACAGAGATACCTTGTCTTGAAAAAATAAACAAAACAAAACAGAAGGGAAAA
TCTGTTTTAAGAATAAATATGTGTATATCTATAATTAATAATAATATATTAATTGGCATTTGTGGTTAATGGACTAATTTATGAGAAGTGGGTT
TGCTAAGCAAATTAATTTATGGGTTATGTGTAATGTGTTTTTTCTTACTAAAAAAATAATGCATATTTATTATAGATATTTCAAACTATTTCAA
AATAGAATTAGTGACGGGTAAACTATTTTAGATCCTTGGGAGTTTTTCAGTTTTGTACCACAAGTAAATTGAGAAATATTAGGTTAGGGATATA
TAGCTCAGTTGGTAGAATGCTTATCTACCACACAAAAAGCCAGGGGTTGATCCCAGAACCTCATAGAACTCATACAAACCTCATATACCAACAT
TTGGGAAGTGGAGGCAAGAAGATCAGAAATTCAGTCATTTTTGACTACAGAGTGAATTTGAGGCTAACTTGGGCTACATGAGACTCTGCTTCAA
TTCTTTTTCCTTCTAGGATCTATGCCTTTTGATATAAGGGAACCTTCAATGTACAGAGATAGTGAAGTTGATATTGTTATTTAAGGCTTACATT
TACCTCCAATGATAATGTATTCCATTTTACTAGTTTTGAGTATTATATTATATAATTATATAATACTTATTCTATTCTATGAGATAGTCACAGG
ATTTAAGATGGAATGGCTGAGGCTGTAGCACAGTTGATAAAGTACTTACTTTACAAGCATGAGGACCTGAGTTTGATCCAGAGCTCACATACAA
ATTTCAGGTGTGCTAGAGCATGCTAGGACTTGGAAAGGCAGAGACCGGAGACTCCCTAGGACTGCTGGCAATTTAGCTTAGCTTACTTAGTGAG
CTCCATGCTCCTGAGAAGCCAGGTCTCAATGGAGCTTGAGAATGTTCTTTAGGAGGACAGTACAAGTTGTCCATTGGCTTCCACATGAATATGA
GCATTCAGACGTGTGTGCACTTAAATAACATTTAAAAATCAAATAATGGCTACAGCTATAGCTTAGTTGTTAAGAGTGCTTGCTGTTCTTCTAG
AGAACTCAAGTTTGTTTGCCAGCATCCATATTAGGCAGCTCTTTATAGCCTCTAGTCTGTTTCTGTCTGTCTTTTATATTTATTTCACTTTTTA
TATTATGTCATGATCCTGAACATTTATATGTATACCATATGCATGCCTGGTACCTACAGAAGCCTAAAGAGTTCATCAGATTCTTTAGAACTGG
AGTTATAGATTGCTATGAGCTATCCCATGTGGATGCTGATAACTAAGCTGAGGTTCTCTGCAAGAGCAGCCAGTAGTCTTACCAGCTTGAACAT
CTCCCCAGACCTTAATCTCGTCTTCACTACCATTGAATTCCTATCAAGAATAACAGCAGTCACCTGCCATGGACACTTTTCAAAGAGTTGGGAG
ATCCTAATTCTATAGGTGAGATCTCTTGAGTTGTAGTTGCAATAACTCCATATTCATGTTCTCCTTTCTAGTGCTGATTATAAACCTAAGAAAA
TTAAGACAGAAGATATCAAGAAGGAGAAGAAAAGAAAATCAGAAGAGGAAGAGGTTAGTAAAAGTTTTTGAGGCATACATTTATTTGTATAAAA
TAGCCTTAAAAAGCATGAGATTATTTTTAATTGTGGGTATCATGATGCACATGTGGAAGTCAGAGGACAGCTTTGAGAAGTCGATTCTCCTGGA
AATTGAACGGGGGTTAAGTTTGACAGCAGGTACCTTTAACTGCTGCATCATCATCTCTTATGTCCTAAAATAGATCTTGAGGCCAAATGAGCTT
TAGAAAATTTTTGGTTTGTTGTAGTAGTTAGAGTGGGGAAATGTTTTGCAAACAAGTTGTGGTTGTGCTGAGTTTGCCAAGATTTTGAAGTATC
CAGATTACTCGCCACCCTTGAGTCTATTCATAGCCTGCGTTGATGTTGATAAAGTATGCCACCTGTAGGTATGCAGAAAAATGTTCATAAGGAA
CAACTCTGAAAATTCTGTCTTAACAATATTTCATGACCAATGAAGCATGCTCTTTCCTCATTTTAAATAAATGTAGCTCTAATTTAATCAGTGC
CCTCTTGGTTGTAGGATGAAATCTAAGCACTGGAGTACCTTTCCTCAGTCTGGCCTTTGCATCTTGTGCCTTGCTTCATTTTGTCCCTCCTGAG
CTCACCTTGCTCTGGCCATACTGGCTGCCTCTGTTTGTTTTTCACACCAGATTTTGTACTTCAGATTTCGGTGTGTAGTACATGTTGTTCTTCT
GTCTGGAACACACATTATTTATTCCCTTCTACTGTTTTATAATCTTCCATAATTTCATAACTTTAGTTCAAGTACTTTTTTCCAAGGATCCTAT
TTGGTGCTTGTGGTAGATATAAATAGAATAATGTAGTTATTTTCTTTATTTTTTTAATTACTTGTATTTACTTGCAAGAATTTTTTTTAAAAAA
ATCAGCAATTCTTTAGGCTCTTAAGTAGAGTTCTTAACTTTATGTTCATTCTGTGAGGTTAATCAATGTTGTGGACAAAATAGGCTTAAATGTG
GTTAGGTTAAAATGTACCAGCAGTCATAATAGTGATAATGGGGCATGGGCTTTTGAGATTGTTATCTCAGGGTGAGTATGGTGACACACACCTC
TAATCCCAGCACTCAGAAGGCAGAGGCAGGAATACATGAGTTTGCGTCTACAAAGACCTTGTATTCAGGAGGCACAGAGAAAGTAATTTGATCA
CTAAATATGCCATTTATTAAGAACTTGATATGCAGGCTGGAGAGATGGCTCAGTGGTTAAGAGCACCGACTGCTCTTCCAAAGGTCCTGAGTTC
AAATCCCAGCAACCACATGGTGGCTCACAACCATCCGTGATGAGATCTGACTCCCTCTTCTGGTGCATCTGAAGACAGCTACAGTGTACTTAGA
AATAATAAATAAATCTTTAAAAAAAAAAGATTTTAAAATTTTTGTATATCAGACCCGCTTGTACCATGTTTGTCTCAGATCCCTGCAGAAGTTA
GAAGAGAACATCAGAGAGAGAGAGACAGAGACAGAGAGATAAGGAGTTTTACTCTGTGACTCCTGGCAATCCTCCTATCTCCCCCTTGACTATA
GGTATTTTTAGACTCGAGCCACCAACAGAGTATTTGAATGCTCTTCTACTTGCACACTTGTATTCCTCATTGTTACTTAAAATGCAATAGCATA
GCATTTGCTTTTTTCATCCTTAAAATAATACTTAAAGACCTTCAAGTTTATTACTACTTTGATTACCATCTTATTACAGACTCACAGGATATTC
TCTAGCCATTTTCATTCATCATATTCTGTTGGTTTTTGTTTGTTTGATTTGAAAATATACTTAGGGTTTTATTTTGTTCAGCAGCAGTTATATA
AACTTGATCAAATGGAAACTGATTTTAGAACTATCTTGATTATTGCTGGAAGAGAAGAAACCAAATAGTTTCCATTCCATAAACCATCTTTCAT
TGCTATATAAAACTTACCAAGTAAAGCCTCATGTGATGCTGAAAATGTAGATCAAAGTTAGGTTCTTCAGAAGAAAGTAGATTCTCTGGAGTCT
GCCTCTTTACCTGTTGAGTCACATTTTTGTTGGGATGTGTTTTGGATTTGTCTTACTCTTGGGTTTGAAGCTTTTTTTTTTTTTTGTTTTTAGA
GTTACACACCCACGGTTGACCTCAAACTCAAACGATCCCTTTGTCTCTGTCTCCTGAATACTAAGATTGAAGGCATATGCTATCCCCAGCCTAT
TTTGTGTTTTTAAGATTGGAATCTGGCAGTCTGTCTTTGAACTCCTGTGGCTAAGTTACTACTGTTTAGACACACAAAGACAGGATACTCTCAA
CTTCGCTCTCAACCTCGAACTTCATCCCCTCACTCCCTTCCTTCCAATTCCTGTCCTACCTCTCTCCTAACTTTGCCTCTCATTTATTGTCTTA
ATCCACTGAGCCCACTTAGTATTTTCTTACAACATGGGTGTGGTCTGAGCATAGTTAGCCTCTCAGGACCTCAATCCCTGAAGTAAACAGACTC
TTCCCTAACCTAGCAGTAAAGGTTTTTTGGGTTTTTTTGGGTTTTTTTTGGGGGGGAGGGGGGGAATAGGGGTTCTCTGTGTTAACCCTGGCTG
TCCTGGCCTCAAACTCAAGAGGTCCACCTCCTTCTGTCTCCCTAATACTGGAATTAAACGTGTACGCTACCACTATGCCTGACTTTTTTTTTTT
TTTTTTGGTATGCTTTTACTATGTATGTATCATGACAGTTCCTGCTACAGCTTCCTTTAGTGCTAGAATCAACAGGTGTGCACTATCATGCTTG
ACTGGTGTGTTTTGTTTTTTTGTTTTTTTTTTTTTTTTTACTTTGCCACTGCGAGCAGTTCCCAGCTGAGAATACTCTTAACTTTGTGTATATA
AGCAATAGTAACTGAATCACAGAAATCTCCTTTCAGATCAAATACTAAATTTTTCTTGTATTTCTTTTCTATACTTTGTTATATTTAAAGAGAT
TAAAGATGAACACACTTTAAAATAAATGAAGAAAATACCCTGGGATTACTGTTAGTTCCCCAGGCTTTCCCTTGTGAAATTCCTAAATAATTGT
TTGCTAGAGGGACTGCGGGAAGTATTTACTCAATTGTAAAATATCAATAAATTTTTCCCTTGATTTCGGGGAGGAATTAGATATACGATCTTTT
CCAGTTCCAGGACTTCTTGTTCATAGTTTTTCTTCCTAGTGGCTTCCAAAAACAGCTTCCAGGCGTGTAAACAATCCTGATTTAAGTGCTTTCT
GTTTTTTTGTAGGATGGTAAACTTAAAAAGCCCAAGAATAAAGATAAAGATAAAAAAGTTGCTGAGCCAGATAATAAGAAAAAGAAGCCGAAAA
AGGAAGAGGAACAGAAGTGGAAATGGTAACATCTCAGCACTGGGTTAAGATCTCAGCACTTTCACTGTGCAGCCCAATTAAACAAGAGTTGAAA
TATTAAGGTTAATTTCTTGACATTTGATGGAGAAGTACTTACAGACTCTTTTGAAGAACAGATAAGTTTTCAGATCCTAGTAATTTACCAGCTC
TAAATCTAGTGGATCTCAAAACTAGTTTCTTCTCATTGAATCGGATTGAATATAGCTTATATATAGAAGGGACTTCAAAATGAATTCATTCATT
CATTCATTCATTCATTCAATCACTTCAAATTGTACCTAGTAGTATTATAATATATCAGTGTATATATACTTTATGTAATGTTTAAATCAGGCTA
AGTCTACCTATCTCATAATACATTCTATCTTTATGCTGAGAACTACCAGCATACTTTCCTGTGTCAGTTTATTATTGCTCTTACAAATGTATTC
ACTATGTAAAGGGTTCTTTTTTTTTTAATCTATAAATCATTTTCATAAAACTGCCAATTTGCATAGGTTTTCAGGTCTATTTGTCTTTTTTCCT
CTCTCAACTGACCTCATAGAGCCAACTTTACTCAGATTTAAGAAAAGCATGCATAAGCTTAAAGTGTACTAGAACACCTGCTACTCTTGCAAAG
GACTTATGTTTAGTTATCTTCTCATAATCCTCTATAGCTCCACCCTCTTCTGGACAAACATCATCTTTTTCCCCAAATAATCCATCTTTTAAGA
CAACCAAAAACAAGAACAAGAATAATTCTAAACAGATAATTCTTTACCAAAACACTTGGAGAATGGCCACTTCATCACCTATGCTAGGTTGAAG
TTTCAAGTCTTTGGGCAAAGAAAAATGTTAAAAAAGAAGCTAGGGATAAATAATATTGTATAGGAATTACTGTTGAAAGACCAGGACTTAACAG
TCTTAACAATGACAATGCTTATGCAATACATACATGCATACATACATAATTAAGAAACCTTAAAGAACAAGCATTATCATATCTTCTCCAAATC
AGAGTCTGTTCTTCATTCTAGACAGCTTCCAGAAATCTCTAGTTTTAGATTTTTATCTACAAATCTTTATTTTTCATAAATTATATAGTCTGTT
TTAAGATACCTGCTAACACCAGGAGACCTTGACTTGACAAAGTAGTAGGCCTATGCTGTTGCTCAGGCTGGCCTTGGGTTCCTGGCTTTTTTTC
CCCTTAGCCTCCCAAGTTACTGGAACTGTAAGCCAATCCCAATCCCATTATGTCCAATACGGAATTCTTTGTGTATTATATTAACTCTTTATAG
GATTTTTAACTTCTGAGCTATTTTCTATTCATTTTTAGACTGTTAAACTTTTTGTCTTTCCAAGGAAACTTTTTTGTTTTTTTTTAAATAAAAC
TGCTACCTAACTTGAATTTATAAGCCTACCCTTCTTTAGTTGGTGCTGGTGTTGGACTGTTAGAAGGCCAACACTACAGTTTTATTTGTATGGT
AGTACCGGTCTGTTTAAACTCAAGGTTTTGTGTTTAGTGATTCATTGAATCTTTGCTTAAATGGTAAGGAAGTTGTTATTTGTCCTGAAGAAAA
AAAACATGTTGAATTTGGAGGAATTGTGAAACATGGCTTGGAATCTTAATTTCAAAACAATGGAGTTGATGACAAGAATATAAAGGGAATGGTA
TTCTGAATCACTCCCCGGTTACTATATGACTATTAAAACAGATGAGTCACTTGTAGGTGCTTTCACATGCTTGCAAACATTTTTCTCTGATTTG
GAGAGTAGTAAATTTGAAGGTTACGTAGTTTCTATCTGGGCATTTGACATCAGCTAGTATAATAACGTTTTATCTGTAAAGTGGTTGTTGATTA
TACAAAGTCTTTATTGTTCCCTCTTTTTGTTTTTCGAGACAGGGTTTCTCTGTAGAGCTCTGGCTGCCCTGGAACTCACTTTGTAGACCAGGCT
GGCCTCCAACTCAGAAATCCGCCTGCCTCTGCCTCCTGACTGCTGGATTAAGGGTGTGCCACCACGCCCGGCTTATTCCCTCTTTATAAAATCT
TTAAGAAAGAAAATAATACTTCTTTCTTACAATGTAATTTAGATGTATATTTACATTTTGTCATCTTATTGTAAATAAGCTGTAATGAGAAACT
CTACATCCATTAAGAGGCAACAAAAAGAAGTTTTGGTGGGACTGAAATAGAAAAATTGGAATCTTGGAGAGAATATTCTATAGAATACAGGGAG
GTACGTCTACCAATACTCACCTTTCCTTTCTCCTTACTCCTGTTGCTCAGCCTGGCCTTGCGTTCCTGGCTTTTTTTCCTTAGCCTCCCAAGTT
ACTGGAACTGTCAGCCAATCCCATTATGTCCAATATGGAATTCTTTGTGTATTATATCAACCCTTTTTAGGATTTTTAACTTATGAGCTATTTT
CATTATGAGTTACCTCTTTTTTTCTTGAATATTCCATTTGATCCATAGGGTTTTTTGTTTTTGTTTTTGTTTTTTAAATTTTGATGTAACCCAA
CTTATCTGGTTTTGTTGCTGTTACTTTTATATTTATTGTATCCACGAAGTCACTGCCAAATTCAGTGTGATGAAGCCATTCCCTCTTATTTTCT
CCTAATGTCCAAATGAGCTTTAGTTTTTATAAATTAGAATTTTAATTCCTGATTCATATAGAGTTAATTTTTGTATATAGTATAAAGGACTTGC
CCCAGTGTCATTCTCTTCAAACTCCAGTGTTGAAACTCTGTCATCTCTAACCATAACATGATGATAATTCCTGTTTAAAATGACTTGCCAATAT
ATGTTGACAGTAATTTTATATAACAGTATTGTTACAGCTTAAACGGATTCATTTATCTCTTACCCTGTGATTCTGAATCTTTAAAATATGGGAC
TTGGAATTCATGGAATTAATTAACAGCAGAACAAGGCAGAACTATCCCAGCACTCAGGAGACAGGCAGATCAAGTTCAAGGCCAGCTTGGTCTA
CAGAGCTATTTCCAGGACAGCCAGGACTACACAGAGAAACCCTGTCTCAAAAGACAACAACTACAAACCCAAACCAAAACAGGAATCCATAAAA
TTCCTTTTGTGTAGAAGTAGCAGTAATAAGTTTGAGAAATTTAAAAGTAGTCTGTATGTATCAGATGGATTCTTTTACAACTCTGAGAGGGGTG
GTAAGATCTGTTTATCAGTGTAAAGGGTGTGTTAGAGACACATCCATATGTAATGCCCAAAATCAGTAGATTTGTACTTGTAAAATACTGGATC
TCATTGGTGAATTTATAAAAAATAAAGTGACACAAAAAAAATCAATCTGATGTTATAGTTCTATACAAGATCAGTTTTATAAAGATAATTGCTA
TAATATTTATTGAACTTTCACATCCAAAACTAATTTTGAAGTGATCAGTAAAAGTAGAAATGTAATAAATTTTAATAATTCTTTGTATATATTT
ACAGTTAATTGTTTTTATATGCTTAATTTTTTTAAACTTTTTTTAAAGATTTATTTATTATTTATTATTATATGTAAGTACACTGTAGCTGTTT
TCAGACACACCAGAAGAGAGAGTCAGATCTCGTTACGGATGGTTGTGAGCCACCATGTGGTTGCTGGGATTTGAACTCAGGACCTTCGGAAGAG
CAGTCGGGTGCTCTTACCTGTTGAGCCATCTCACCAGCCCCCTATGCTTAATTTTTAAAAAATAAAATGTTTTAGAAAGATATTTATTCTGCAG
TTTATTTAGATTTTTGTCAAGTATCTACGGGATAGTTCCCTGTATTTCAGAAGTGCCTTAAACTTAGTGCACCTCCATTAGAATCTCCTCAGTC
CTCTTGCGTTTGTATTGTTGGTTTTCTTGAGTCCAGGTCTTACTATGTAGCCTTGGCTGTCCCAGAACTCTACCTTCTGCTGGTCTCAGAATTC
CACACTCTGGAACTGAAGGCATCCAGTCTGGTTGTGCTTTGAATTTTCAAATAGGGCTGCTTTCTTCTAAACTCTCACCTGCATGTGCCATCAG
TTTAGTAATGCCTCTTTTGCATGTACCACTGTGGCATATTTTATATATAAATAGTTTTATATGGGTTTTTTGAGTGATGGATGGTCTTTTAATA
CTTAGTATCTCTAAATTGAAACTGTTGAAATGAAATTTTTTTAATTACCATATTCTTTCAGTTTGCTTGAAATTGCTGCTCTTTTCAGTGAGAC
ACAGCCTTTGAATGGGCACCACAAATTGATTAGTTAATTATTTTGAATTTAATACGTGAGGATCTTTTTACTAAGCCAGGTGATCACTTAATGA
GCTTGTGCCTTGTCTTTGAAAGGTGGGAAGAAGAACGTTATCCAGAAGGCATCAAATGGAAATTCCTAGAGCATAAAGGGCCTGTCTTCGCTCC
ACCATATGAGCCTCTGCCAGAGAGTGTCAAGTTTTACTATGATGGTGAGTTATTTCAGGTTGTTTGAATTTTGGCTAAGAATGGTTTGTTTCTA
TTCAGGGTTAGAAAATGAGGGCACTGCCTATATTGCACAGTCCTAGCTAGTTATTTCACTTTCTTTTATTGTCTATGTGTGGCTTGGGAAAAGG
AATCTCAGGTGTTTAATTCAGAAGGACCCCTTTGCTGCTTGAAAAATATGAAAAAGGGGGCTGTTAATGTCTTATCTGTAGTCTAAAAAGGTTT
ATCTGTTTGGCTCACAGCCTATAAAATACACATTGAAGGTTTTTATTTTGAGTGTGTAACTCAATAGGAAGCTGTTCTGTATTGAGTTCTCATT
TAAATAGTAGCACTTAGTGATAATATAGTATGCCAACATGCTGCTTATCACTAGAAATTGTATTAGCAAAGGATTAGTGGCCATCATTCAGCTT
ATAGGTAAGAAACCTAAGCTATGTCACTCTAAGTAGTGCAACTGTGCCTTTGACAGGTGAATTTTAAAGAAACGCTCTGCCTAGGTCTCTTAAT
TTTATTTTTTTGAGACATTTCTAACTGTGTAGCCCTGCCTGGCCTGTAACTTGATAATATAGATCAGCTGTCCTTGAATTCAGTAGATGTACCC
ATCTGCTCCTGAGGCACATGCCACCTTATCTGGCCTCCTTGGGTCTCTTGGAACTTTCTAAAATGTCTGAGAGTTCCACATCTGTGTTTTTGTC
CTTGGGTTGAGGGATCTCAGAGTAGCCTCTCACAATCCTTCGACTCACTGTGTATCAGGTAGTAATGTCCAACTCACTCCTTCATTCTAAGAGT
TCTGTCCAGAGATGCTAGAGTTCGTTCTCAAACTCTCTATGTATTTATAATTCACCAGGTTAATAGATGAGAGGTAGGACTGGGTGTTTATGAA
TCAAATAAATGTATTTACTAACAGGGAGAGACACTGGACACCAAAATAGCAAAACCATTCAGAGCTATTTGGTGTATCTCTTTTTTTTTTTTTT
TTTTTTTTTTTTTAAATTTATTTATTTATTATATGTAAGTATACTGTAGCTGTCTTCAGACACTCCAGAAGAAGAGGGCGTCAGATTTTGTTAC
AGATGGTTGTGAGCCACCATGTGGTTGCTGGGATTTGAACTCTGGACCTTCAGAAGAGCAGTCGGGTGCTCTTACCCACTGAGCCATCTCACCA
GCCCCTTGGTGTATCTCTTTAACCGCCAGCAATGGCTTTAGGAGCATAGATGCCTGTGGGTTTAGGATGAGACGGGTTTATTAGCATCCAATAA
TTGAAATCTGTATATGAAACAACTGAATTTTGACCTGGGTCTCATCCTTAAGATATTTTATTTCTCTCTCTCCCTCCCTCTTTTTTTTTTTTTT
TTTTTTCAGATAAGGTCTCATTTTTGTAGTCTTAGCTGGCTTTCATCTCAGACGCCTGCTTGTCTCTGCCTGAGTGCTGGAATTAAAGATTCCC
ATCACTGATTACTAATAATACCCAACCTGATACCTTATTTTTACAAACACACCCCCACATACCGTCATTCTCCAAAATCTAGGGGTGAAAAAAA
TGAAAATGGAAAAAAATCACATGTTTTGGGAAATGGATGTTCAACTAATATTTTCGAAAGGTTCGGAAATGTTTATTGAATATGCTTGTGTTCA
GACACCCAAGTCCCAGATAGATAGAGACTACTGATTTAATCTATGTAGGAAGTCAGTGGCATAAAACACTTGGTGGTGTGACTGGCAAGTTTTT
AGATTAGCTAACCAGTTTGACCAGAAACTGTAAGACACCCTACACACTAGATCATTCCTGAAGATTTACGAAAATACACAAATTTAAACTTCCT
AGTGAAGAGAAAAGGCCTTAAGACCTTGAAAAAGACAATTGCTGTATTCTGTAGCCTGACCTGAGCTCACTAACTTTAGACAGCTCGCTGGAAA
AGCCTCACTGTGAAAGCCAAGGTGATGACGATAGAAAGTAGTTTTTTTTTTTTTTTTTTTTTATTGTGGGGTTTCAAGGAAACTAAGGCCTGTG
GTCCCTACCTCTAGTTTCCTTTTCTCTGTTGGTCCTCCTAGGATTTCCTCCCTTTGGTTATAAAACTCCTCCAAATAAGTCAAGAGTGTTTGAC
TTACTAGTGCAAACTGATAAGTAAGTACTGGTAGGCAGGACATTGCTGCCTTGCCCTGGACTTCCCCCTGGCCGCCTCTGCACAGGACATGAGT
TGGCTGAAGCACAGAATTGCAGCCGCTGTTAAGGACCTGCCATTTCAACTCTGACTCAACAGTTTCTTTTTGTTTCCGGGAGCTCAACTTTTCC
CACAAAGTACTACGATGATTGCTGGGAACCTACACACCCTCCACCACTCCGGTGCTATAGCTGTTACTTAATCCTTCCACAGCCCATTTAAAAC
AAATCAAGTAGGTTCTTTGTGGGTGGGACAAAGCCAGTGCAGTGCAAAGGCTCCCCACCCAAAATAAGAACACTATTGGGGAATATTTAGTTTA
TGAAAACAAACATACCAGGGAACACATTCCTTAAAATTTCCTTATCTATTTTTATTTTTTGTTTTGAGATGCTGATTTTGTAGCTGAGACCAGT
TTCAAATGCACAATCCTCCTTTCCCAGCCTTCCGAATGCTACAATTATAGGCTACTGCTACCTTGTCTTTTTGCCCCCATTTGTTTGTTTGTTT
GTTTGTATTCCCCTCCATTATATTTGAATCATGTTGCTGTTTTGCTGGCAACTCTTGTTCAGACTGAGTGGGGAAAACAGGAAACACCTTACTC
AAAAAATCCTTTTCCAACACAGAATCTTGAGTGTGCTGAGGAAGAGCTACACCACCTATTTGCCTTTTAGGTTATGTCTTCTAAGCTGATAATG
TACCTGTAGCACAAGTTTGTTTTGTGATGTATTCCCTATGTTTCATACAGTCTGGTTGAGCAATACTACCCATTATGAGATGAGATGTCTCTCA
ATTATTACAATGAGTAGGCTTCTGGTTAAGTTTTAGATTTCTGTAATAAATTTTAATTCTTTTGAATTATTATTAGTACTTACATAGTTGTAAG
TGTGAGATATTAATCTCATTTAGAATGCAGCAAAATGCTATTGTGGCATAAATTGTTTCTATATAATATTATAAAGTTGATTTTTTTTTTTTTG
AGAAAAGATAGAAATTGGCCCTAGGAGTGCAATATTCTCTTTGAATATTTTGTAGATTAAGCTTTTAAGTACTTTTCTTTCACTGTCAGTTATG
ATCTATCTGAGGACACAAAATCACTTCCAGGATTTATTAACTATTGTAGTGGCTTTCCTTGTGAACCACAGGCTTTTTTGGACAGTCTTTACAT
ATACTTACTTGCTGCCTAACCCACCCTCACCTCATGCTGTGGAATGATTAGACTTTCCTGAAAGAAGTGAGTTCCTGGTGCTATTCCCAGTGTT
TCAGGTGCATGTGTACCTGTATTTCACTCTGAATACACTAGTGCTCCAAAAGTGCTTTTCTGGCTGGGAGGCTGGCTCAGTGGGTAAAGTACCT
GCTGTACCAGCCTGGTGTCTTAAGGATGTTTTTTTAGAACCACATCAAAGTAGCACACTTGTCTGCAATTGTAGCGTGTCCCTGTGGTGAGAGG
GGAAGTGGAGACGAGAATGGATGAGTCCGTGCCAGCTAGCCTCTATATATATATATAGCAGTGAACAAAGAGACCTTATCTCAAAATAAGATGG
AAGGCAAGGACTGACACCTAAGGCTCTGACCTTCCATGGGTACTGTTGTACACATACAACCCCACACATAGACATACAGTGCAGTTTTTGATTC
TAGGTATAGGACTGAGGCCCAAAACTTGGTATTTATAACAAACTTGCTTGTGATGGTTTTGGTCTGTGAACTACTTGAGTAGCAAGGTTCTAGA
GGAAGGTCTTATTTACGCTTTTTATGTGGGAAATCTGGGCTTGCCTGCTATGAATATATATTCTTATTCATATTCCACCCACGTAACTTTGAAG
AAGAAAGATGCCTATTTCTTTTCCCCACAATTCCTTTCTTCCACTCCCTGCCCCTATCCTTTCCTCCCCTTCTTGAACAGAGGCTGCTTAGCTT
AGCAGTGGCAGGAGTCCTTCTGAGGATTTGATTTTTAAAAACCATTGAAGAGGTGTAAAGGACCCTGCTTGCTGAAAGGAAAAGGAAGAGTGGC
TGGGCCTTCCCCAGGTCATCCCTGTCCTGAGCAGGCACCTGGGAACAATTAACTGGCATAGTCTCCAAAAAAAGACCCAGGTAAATATCTACTA
AAGAGAAGGAGAAATTTTTAAGTAGATCTCTGACAGTATGTTACTCTGTTGTTGTTATCACTGTGTGTCAGCAGACCTTTGTACACCTTGGAAA
GGTGAGCCATCAGTCATGACCTCTCCAGTGTTCAGTGCACATAGAATATGAATAGTGTGCCTCACTGTTTGGAACCATTTTCTTCCTTCAGGTA
AAGTTATGAAGCTGAGTCCTAAAGCAGAAGAAGTAGCTACATTCTTTGCAAAAATGCTTGACCACGAATATACTACTAAGGAAATATTCAGGAA
AAATTTCTTTAAAGATTGGAGAAAGGTATTGTGGCCCATGTGTTCATATCTTAATCATTTATAAAAGTGCTGACTTAGAGGTATAGCTCATTTG
TTAATGTCTTGTTTGTTTAAAAAATGCAAAGTATAGTTTAATGTATATCTTTTTTCCCTTACTATTTTAATGTGGTTTTTGTTCAACAAGAATA
TATCCCTTCTCAGCTTTCATGTGACTGCAGGAAGGAGCTTATCAGATTAGGTTAAACAGAAAACCTCAAATCCAGTTGAGCAAAGGAAGCATTG
TTTCTGCAGGGACTGTCCTCTGATGCCTTTACACAGGGTCAGTCTGACCATCTATTCTGTATTGATATAAAAAAGTACCATTGTTTTCAATTTG
GATAATTTTGAAAAGAGGTTGAATTAAAATTCACCATCCAAGGGCATTCTAAATTTAATAAGAGCAGATTAGTGTGCATGGGAATAAGAAAGTA
TTTCTCTTTATTGCTAAAGTAATTGATTAATATAGGTCTCTTGGATTGGTGTTTATATGTTTGAGTTTGTGTTTTTGTCAAAGATCGCTAAAAA
ATGAGTAATTTTCTTTGATAAATGACTAATGATATTTTATATGAAACTAATCATCTTATAACAGAGCAGCACCACTCCCCAGAAGCAATGGTTA
TTGAGGTTGGCTTCCCTTCTAGACACCTTTGATTAAAGGGGAAAAAGGATGAATTTTGAAGAGACAGGAAGACTTATTAATATATTTAATTTTC
TTAACCTTTTTACTCAATTAGGAAATGACTAATGACGAAAAAAATACGATTACCAACCTAAGCAAATGCGACTTTACACAGATGAGCCAGTATT
TCAAAGCGCAGTCAGAAGCTCGGAAACAGATGAGCAAGGAAGAAAAATTGGTATGATCGTCTGCTAAACTAGAAAATCTTGGATAGTGATTCTT
TGTATAATTTTTTGCTTCTTGTGACACAACAGTGAATTCATTACTTTTATATTTTTCTGTGTTAAAGATTTACTTAGTAAAGTATAATTACATG
TATGTGTTGTGCTAACTGGCAGCTATCTGTGTTGGCACATGCCTGTAATCTCAGCATCTAGACTTGAGCAAGAACTATGATGTCTCCACAAAAC
AAACAAAATATTGCATTAGACTTCTTTAAGTTCATTTTATCTGTAGTTTTCCAAATCACTGAAAAAAAGTGATTCTGAAAGTGTGCATTAAATT
TTACACTTTCACCATCAAATGCATGTTTTATGGACTTATCCCCTACCACAAAATAAACCAAAACAAAATCCATGACAGTGTTGAAACTAAAAGG
GAAAAAAATTGCTTTATACCCTTTTGTCAGTCTGTGTTTTGCATTCAATCCTTCTTTACTACCCAGCATTTACCTCTCCTCTTTCTAAGGCTCA
CATTTAAAAATACCTAGGAAGGGCTCAGTTGGTACAGGGCTTTCCTAATGTGTCTGCAACCATGATTTTGCTTCTCAGCACTGCATAAAACCAT
GTGTAGTGATGGTACATGCTTAGAGTGTCAGCCCTTGGGAAGCGGGGGATCAGAAATTCACTGTCATTCTCAGTTACATAGAGAGTTTGAAGAC
ATCTGGGGTACATGAGACCAGATACTTAAACTAAGCTACACTGAAAATAAATGACATTTATAAGTCTATACCCTTTGTAATCAAGATTAAGTGT
TGAAGCACTAGTTCACTGGTAGACACAGTTCTGTAATACTACAAAAGACAAAAGTTGAGAATCTTATTTGTGTAAAGTCAGTTATTCTTGATAG
ATCTTTTGGCAGTTTATTTTATATATCTGTAATAATATATGTACGTTTAAAATACACTACACATTTTCCTATATTTTAGTTTGTTTCTGAGTGA
TTTTAAACTTTACCTTGATCTTTCTTCTGCAAGAGATAAAAATATGTAATTCTTAGCATGTACCAGTTTGAATCCATTTTGTATACTTCCTTCC
TCTATTTTAGAAAATCAAAGAAGAAAATGAAAAGTTATTGAAAGAATACGGCTTTTCTGTAATGGATAATCATAGAGAGCGCATTGCCAACTTC
AAGATAGAGCCTCCGGGGCTTTTCCGAGGCCGAGGGAACCACCCCAAGATCGGTATGCTGAAGAGAAGGATCATGCCTGAGGACATCATCATCA
ACTGTAGCAAGTGAGCACAAAACTCTTTAGGGGGGAGAAGTGGGTGCAGGGCTATTTCCTCAAGGCTGGTTATTGGGACTATTCCTACTGTTAT
GAGAGCGGTGCAGCGCTTGCATGGGCAGGTCTCCCAAGTTTGTTCAGGATTGAGGGCAGGATCAGAGGGGACATGGTTTGTCACTTTTCATTTC
ATTTTCTTTCTGAATGGTTATGTTCATAGAGACGCAAAGGTTCCTTCTCCCCCTCCTGGACATAAATGGAAGGAAGTCCGACATGATAACAAGG
TTACTTGGCTGGTCTCCTCGACAGAGAATATCCAAGGTTCTATCAAATATATCATGCTGAATCCCAGTTCACCAATCAACGTAAGCATAAATCT
AGGCCACATCCATTGGTGGAGCTAATATAGTAATCTCATTCTCTTTTGGTTGTATTGTAATGTGCTTTCTAGACTCATTTTAACAAATGATACT
CATTTCAACAAATTAAAAACCTACCCTCTCCCTGGCTTCATGGTTAGTTAGCATTACCTAGTAAACTCTTATTAAAGATGTTTTACATTTGTGT
CCTATAGTAATCTTAGCTGTGGACATTCTCTGAAATCCCATTATGTTCTCAGTGAATGCATGTGGTAATAATGGTTGTCCACGTGCTACTGTCT
GGTGGTCAGGATGTGATGAAGTGGCTAGTAGGCTTATGTGAGAAATGTAGATACTATTTTTCTCTAAATATAGAGACAACTATAGGACCTAGAG
CTTTATATACTTTATGTTTTCTGATAAAACATACTTTGTCTCATCTGAGTAGGTTCCTTTAGGTTGTGATTGAAATATTGCTAGCCCACCATGT
ATACATACTCAATTGTTTTCAATTGTTAGATTTACTGAGAGGGACTGTCTCTGATCTTGTTAGCCAGGTAGCTATAAACTCTGTGTTCTTTAGC
ATTTTATAAGCTCCTTCCATCAGACTTTATCGATATATGAACTACAAATAAAAGAGACATTGGGTTCACAAAAAAACAGCCAAGAAAATTGGCT
CTGTGTAGCCTATAGAACTGTTTTCCATTGAAGAGTCTAAAGCTAAAGAAACCCTTGTTCCTAAGGATTTAGCTTCTGTTAAGATCCAAGAGTC
ACAGATGAAAATGCTATGTAAATTTACATGCTTTCTAGCTTATATCCTGTGTTCTGACATGTGTCTCTACCTTCTTAATGAAAAATACCATCTG
GATACTTTCTTCAAATAAATTCGAAAATAGTATCTGTTTCAGAATAAGAAAATAATCAGAAGGGAGAACTGAGACTCCTTTATTCCTTTCTTCC
ATGAGATACTTTACAGAACTACAGAAGTGGCTAAAGAAACCATGTTTTTTTTTTTTTTTCTTTATGGGTGGGCAAATTCTCTAAGGATCCAGTT
GTGTTAGAAGAAAGGTAGTCCTTCTTGTACATAAGAGTAGTTATTCTTAACATCAGAATGGACTTTTTAAAGGAAATTAGATTAAAATGAGCTC
TTTAAAGAGGTCTTCTAAATAGACCCAGGTGCTTATGGACAAAAGAGGAAGGTAAGTTGGTCTACAGGGATCTGACAAGTACCACACAGTGTCA
AGTAGGGCTGGGGAGGAAGCTCAATGGATAAGAATCCTTTTTGTAAAAAAGACCTGAGTTCACCTCTCCAGCACCCACATAAAGCTACACATAG
CCATGCAGACTTCAGTGTTGGGAACTGTCAGAAGCTAGGAGCTCCTGGCTGATTGTGGTCTTCAGTTTCTTAATCAGGTGGAGGGGCTAACGAG
ATAACTCAGTGGCTAAGTGTACTGGCTATTTTTCGTGATTGCCGGTGTTTGATATTCCTAGCGCCCATACATACAACAGTATGAGGTAGAGAAT
CTCCTCATGCTTACAGAGTTGTATGCCTACATGCATACATACATACATACATGCATACATGCATACATACATACCACACCCTGTAATGTGCATG
TACCACATATACCACAACTACCAAGTAGTAGTGGTTCTATATATAATATTATTAATATACTAAATGTCACATTAATGGTACATTCTATATATAT
ATATTCACCACAGTCAGTATATTGTCAGTCTGTGATGAGTATTATTTTAGTGTTAAAAAGCTTTTCATGTAAGTTGACATTTAATAACTATTAC
AGGCATATATTCACAAATAAATTTAGTCTGTGGGAATTAAAAAAAAAGTCCTACATCTGATAGCTGGCAACTCTTCTCAGTCTTCTTAGAATGT
AATTTAGATCTCCAATCTCATGCTTTCCCATGACATCCTGGTGTCAAATTGTGGCCAGTCTTAAATAACATGCCCAAGAAGACGGTCTTCTGTT
CCGGGAAGAGAACCCTTTAAGGACTTTGAATGAAGGCTCTCTTAAACATGGGTTACCAATTTCAGCAAGATTAGCAAGTTATTACACCAATAAA
TATAAACTACGAGAAATGCTGCTGCACAGAAGTATCAGTACCATTTAAGAGCATACCTCAGTGCTCAATCTTAGCTCAGTTTCAGGAAAGGCTT
TTCCACAAAGATAATATTTAAGGTTGTATTTGTGAGATGAATAGGAATCTCATTTGCCACTGGAAAGAAAAACAAAGGGAATTAGCATTTCAAA
GAGGGAATGAGAACTTTTCAAGTTTCTGCCATGAGAAAGAGCTCACGTGTTTGGAGAACAGAGTGCTTTGAGCAGTAAGAGAAATGAAGAAGGC
CCCGCAGAAGCAGACAGGCCAGGACTTCAGTTCCAACTTTTTAAATGAGTCTAAAAGGAAGCTACCGACCTAGACAGACGTAAAGAAGAAAATA
GGACTGGAGTGGTGACATAATTGATAGTTTTGACTACTACTTCGTCTATGTAGCCTGTATGTATTGAACACAAACAGGAAGTTAGGAGGCCTCT
AGATAGCTCTTCTTCAGGCAAGAAACTGTAGTGATCAAGGCAGGTGATAGCAGTGAGAGTGGAGGAAAAGGAGTAGATTTCAAGTATGTGGCTG
TTGTGTCCTCCTATAGGCAAAATCACAGGAAACTGTAGTTCTCACAAGAGTGAGATAGATACCTGCTAGGCAAGCCAGAGCAGCACCCCAAGGA
CAGAGGTGCTTAGTCTAGGCAGTAATAACCAGATAACTGCATAGTAAGGATTTTTCTAGGTTTCTTCAAGATGGTTTTGACCGTAAACTTGAAA
ATCACCCCATGATAGAATGCTTGACTTGATACAACTGGGATGACATTCCAAGAGTAGTCATCACAGGTCTGGAAAGGTGGAACTGCTGGCAACC
AGGGGTGAGGAGGCAGTGCCTGTGGCTTCTTGCAGTTGCTCTCAGTCTTGTCATAGGCTCCTTCATCTAGCAGAATAGCAGTGGCTTCAGAGGG
AAGAATATCATAGCACCATGAATAGTGTACTGCTACCAGAGAATTATTCCATTCTTAAATAAAGACATAGGTTCAAGCTTTAGGATTTTTTGGT
GGGAGGGGGCATGGGGTTGTTGTTGCTCTTGTTGTTTTATAGGCTTCTTTATTTTTATGTGTGAGTATTTTGCCTGCATAATGCATCCATACTG
TTTTATGTCCCTTGTGTTTGCAGAGTCCAATAAAAGGTATCAGATCCCCAGAACCAGAGGTAGTTAGGGACAGTGGCAAGCTCTTCATGGGTGC
TAGGAACCAAATGTACTTCCTTTTTAAGGGCAGAGAGTGCACTTAGTCTCTGAACAATTTTTCTAGGTCTGAGTTTTCTTTTAATGAAATAATT
CAAATATGTAAGAATGTCTTGTCTGCCATTGTCAGATCTTAGCACTTTGTCCAATATACTCAAATCAGTTAATTAAAAAAACTACTCTTGAAGG
GCGGTTGGTGAGATGGCCCAGAGGTTAAGAGCACTGACTGCTCTTCCAGAGTTCCTGAGTTCAAATCCCACCAATCACATGGTGGCTCACAACC
ACCTGTAATGAGATCTGAAGACAGCTACAGTGTACTTACATATAATAAATAAATAAATAAATCTTAAAAAAAAAATACTCTAGTGGCATAGCCT
TTATTTAACCTAATACTTGGGAGGCAGAAGAGTCGCTTTGAGCTGGGCATGGTGGCACACGCCTTTAATCCCAGCACTTGGGAGGCAGAGGCAG
GCAGATTTCTGAGTTCGAGGCCACCCTCGTCTACAAAGTCAGTTCCAGCACAGCCAGAGCTATACAGAGAACCTGTCTCAGAAAGAAAAGAAAG
AAAGAAAGAAAGAAAGAAAGAAAGAAAGAAAGAAAGAAAGAAAGAAAGAAAGAAAGAAAGAAAGAAAGAAAGAAGAGAGAGTGTGTGTGTCGCT
TTGAGTCTAAGGCTATCCTGGTTTTTGTAGCAAGTTCCAAGCTACCCAGGGCTACTAGGGAGGCCTTGTTTTTTTTTTTTTCCCTAGATTCTGA
GAGATTTCACATGTTCCATCAAGTGTTACCATGCAAATATACACATGGAGGATTTATGGGTGGCACTAACTGTTCATAAAACCAAAAACTTGAA
GTAGTAGGGGGGCATGGGGGACTTGAGGAGAAATGGATGATAGATATCATTTTATTATATAAATGTATGAACATTTCAAAAGTAAAGATAAAAT
ATGTGGCAATTCAAATTATTTTTTAAAAGCTGGGCATGGTAACACTTGCTGGTAACTCCAGCACTGGGGAGATGGGAAGGGATCCCTGGGCCTG
GCTTGTCCAACAGTCTGACCTAAGTGGTGAGTTCTGGGCCAGTGAGAGACCCTATTTCAGGTACAAGCCTCTGAGCAACCAGCTTGAGTTTGTC
TTACTCACAAACTCAAAGTATATTAGCAAATGGCATCTTCCTCAGTACAGGTCCTGTGAAGAGTTAAAAGCAAATGACTCACTTCCTGTTCTGC
CTATATTATTGAGTGGATAAGTATCTACATTTAAAAGAAGTATGGTGGCCACTGGTTTATAACTATCACTAACTAGTTTTTTCTCCATTATGGA
GAAGTTGCCAACTAGCGAAAGAGGAAAAGGAGTCAGAGACTCCTACAATATCTACCTTTGTTTTACCAAACTGATTTTCTACTCCATACTAGTA
ATGTGCTGATGTAGTAATAGAATTTGAGGGAGCACATTTCTGCCAGTGTCAGGAAGGTACTTTTTATGATGGCTTTGATGTGACTCCCTGGAAG
ACCTAGGTGGGTATTTTTTAGAAGCAGTCCTAAAGGGTTTTGAGTCTATTATCATTTTATAGGTTCTGATCAGCAGCGCTGTAAGCCACCAGGA
TAAGCGTATTTGGGAACTAGCCCTTGCCTGCTAGCCCTTCACAGGATGTCTCCTCTTCTTGATCCTCATCCTAGTGTTCTAGAGAAAACTTTGA
TTTGTTCCAGGACGCCAATCATGTCAACCAAACACTCTTTAGTCGTTTCTTCTATAAATTACTTACTTGACTTTCTCAAACTTTGTCTGCCTGA
CCACAACCAATGCTTATGTGTTTTGTAGTGGTGAAAAGACTCCACTCCCGGCTAGATAGTGTATTTTACTGAGTCTCTCAGGTGTCTTGGTGGT
GGTTTATCTAAGAAATTCTGCTAGAGGTACACCTTGCTTTGTAGCTTTGTACCAGGAGCATAGCTTGTATTGTACATTTTCT;ATTTATTCTCT
TAATGTCAAGTTGCACTTAGAGAAATCAGAGAGAATTTTCAGCTTCCTGAAAAGTTATTGAACCCCTTCTTTAAAAGCCAAACAAGCCCATACA
AATAGTACACACTCAGTTGTGAGGAAATGTTGACGACTCCCTAGCACATCCAAAAAAGCATTACTTTCTGAGCATTTGTTAAGATCTGGAAGTA
AGCCTTTTTATTCCATGCACCCCCATGCTTTCATGTGGTCCTATGGGTTAGGTCCTTTTGTCTTAGGTGGAAAACAATATACAAAAAGAAATCA
CTACCATCTCCTCTTTGTTGTAGGATCTTTAAATCTCATGTCTGTGTAGGGGCATGTGCGTTTGAGTCCTGCAGTGTGGGAGGCATTAGATTCC
TCTGGAGCTGGAGTCACAGGTCACCTCTGATATGCGTATTGAGCACAAGTCTTCTGGAAGAGAGCAGCATGCGCTCTCAATTGATAAGCCATCT
CTAACCCCAGATTCTTAGGGGATTTTCACCTTGTAAAATTCTGCCTTGTGGAATTTTCCTGATCTTTCTCACTTGACATTATTGATCCTTCCTT
TCAAGTCCTTTTCTACTCAGGGTGGTAACAGAAATCTTTTGTTGTTTAATATCAGTCTTTATTTTTTTTATAATATTTTTTATTTAGTATTTAT
ATATTTTACATAATGTACAACTCCACACTTCCCAACTACTACCAGATTCCCTACACACATCATTCTCTCAACTGTGACTCAGTTGAAATAGTGG
TGTCCATATATGCATACATAGGGGCTGTCCAATGGGGTGTGGGAAAGCTACCAGGTATCACACCTCAAAAGAGACCACTACAAATACACACTAG
CAGCTATCAAATGTCAATAGCTCCTCAGCTAGGAGTTGGGGTTTATGTATCCCTCCCCATTTCATGATGGAATGTTGATACTGTGCAAGCAACC
AGAGCAGTTCCTGAGTCCAGTGTGGCCCTACCATGTTTAGAAGACACCTTGGCCCCACTCAAGTTTGTACCTCTTTAAGTCTTTCCATCACCTT
TTCCGATGCTTCCTGATTTTGAGGTCAGAGTGTGTCATGTAGGGGTTCAATTCAATGCCTGAATATTTCACAGACATTCTGTTCTCTGTAATTT
GACCAGTCGCAGCCCTGGTATTAACCAAATCCATTGCACAACTCTTCTGATGAGGACCAAGTCTGGGGGTGTACAGATATGTGTTTAGAAAGCA
GCCTGGTGCCATGTCCATTTAGCAAAGTCTCATCAGTGCTTTTCTTAACCCATCAGACCTGTGAGTTCCCCAGCTATAGGTTCTCAGATAGGTT
TATATTACCAAGCACGTGTGACATTTTCATGGGCTTTAGGATCAGATCTCAAACTTTATGCCAAAAGCTCTTTTGTTATGTTTTTAAAGAAGCC
AGGTAGTCGTGGTTGGTGTACACCTTTAATCCTAACATTTGGAGGCAGAGGCCAGCAGATCTCTGAGTTCAAGGAAGCTAAATCTATGGAGCTA
GCTCTAGGACAGTTACGGCTATATAGAGAAACCCTGTCTTGAAAAACCAAAGAAAGAAGGAAGGTGTTCCCAGAAACATTTTTTTTCTCAGTCA
AATCAGAGACCATTTTAAATGCAGAAGTGAGAAAATGGTGAATAATTACGCCTATTCATGGAAGCTTTTGTCTTCCTTGGTAATTACATGTTGT
TGTCTTCGGTTCTGTTTCTTTGATAGGTCCCTGAAACCGCTATGGCTGTCTTCAATTTAATGGAATTCAGTGCTAGCAGAGAACTGGAGCATGC
ATTCCACACCAAGGCTGTGGTGCTTCTGAAGCAATGGAGATGCTTACATCCTTTATGTTCACAGAAGTCAGTTTGGGGGCATGCTTGGTGGATC
TCACAAGAGTGCGAACCATTTTTTAGATAGGAAAGCTTTCTTAGCAATTATACAGGACGGAGTCAGTAGAATAGCAAGTATTCTTGTCATGTTA
CATGCCCCAGACTTTCTCTTCAGTTCTATGCTTTTCCAGCTTTGGTTAAAATCAGGTTCCTGTTTTGGGGTTCAGAGCTCTAAATATTCAATGT
GTTGCTCCACTGCACCGTCAGAAAGACTGGCAGAAGTATGAGACTGCGCGGCGTCTGAAGAAGTGTGTGGACAAGATCCGAAATCAGTATCGGG
AACACTGGAAGTCCAAACAGATGAAAGTTCGTCACACACCTGTAGCACTGTACTTCATTGATAAGGTGGGGACATGTTCCTTTATCCACATTGG
ACAGTTCTGTTTACAGGTATAGGGAATGTCTACAGTATAGGGATCAGGAATGTATATGTGCTGTGTGACTTATAATCCAAATTTACTTAAGTAG
GAAGAGTAGATGTGGATACAAAGAAAGAGGCATGTACAGTATGTTGGCTATTGAGCAGTCTCTATAACAAGAGGATCTCCATAAACTGAACATT
ATTAGTTAGTTTGTTTGTTTGCTTGTTTTTGGTTTTTCCAGACAAGGTTTCTCTGTCTAGCCCTGGCTGTCCTCCAACTCACTTTGTAGACGAG
GCTGGGCTCAAACTCAGAAATCTTCCTGCCTCTGCCTCCCAAGTGCTGGGATTAAAGGCGTGTGCCACCACTGCCCGGCTGTTTGTTTTTTTAA
TAACTGTGATGCTCAAGATTTTTGACCATAATCTGCAAAAGTACTTACTTTATGTCAAAATCCAACATAACAGTTTTATTTATGTGAAATTAAA
TCGAAAGTTGCACAAACCACTGGTTAACTAGTAGCATTGAATTCTGGTGGTTATTTCTCATTTCATTCTTTTAGAAAATCACAATTATAACCTA
TACAATTCTTAAAAAAGTGTGATTTATGATAATTGCAGTTTGAAAGATAGTTTTTTTAGTGCTCAGATTTTTTTCTTCATTTTACACTTAAATT
TTAGCCCAAATTCTGAATTCACACAAAATTGATAAGTTTTTAATCTGACTGTTCTCATGAAAATATGGTCTCATTTGTTGCCTTGTTTTAATTT
TTTTCTTCTTCTATCTCTACTCTAAGGATGGAGTAGAGAAACTTGGGTCTCTGGGTATTCTGTGACTGACCTACCTACCTACCTACCTACCTAC
CTTCCTTCCCTCCTTCCCTTCTTACCTCCCTCTCTCCCTCCCTCCCTCCTCCTTCCTCCTCCTCCTCCTTCTCTCCCTTTCTCTCTCTGTCTCT
CTCTCTCTCTCTCTCTCTCTCTTACTCTTTTATTTGTTACTATATTTAAGTACACTGTAGCTGTCTTCAGACCTGAAGAGGATGTCAGATCTTA
TTACAGATGGTTGCAAGTTACCATGTGGTTGCTGGGCTCTTAACAGCTCAGCCATCTCTCTAGCCCTCTGACTTTCTTATGAGTTGAGTTTCTA
AGTTATGTAGACTGATGTTCATTCCATCTACAAAATTGGGGGCTTCTGGTTTAGAGTGTCATTGAGATTATATGTGGTCTATGTTTCTTCACTC
ATCTCTTTCTGAACAACTCTGTTCCCTTTCTTGCACAGCTTGCTCTGAGAGCAGGCAATGAGAAGGAAGAAGGAGAGACGGCAGACACTGTGGG
CTGCTGTTCACTTAGAGTGGAACACATCAATTTACACCCAGAATTCGATGGTCAGGAGTATGTCCTGCAATTTGACTTTCCTGGGAAGGACTCA
ATCAGATACTATAACAAAGTCCCAGTTGAGAAACGAGTAAGTTCATGTGGCCTGTGCTTCTTTCTCTAGCTTGCTCTTTTTACATGTTTCTTTA
TTCAGCAAATATTTTTTGAGGGCTTTTGCATGTTCTGCATTTTGATGAGCTCTAGTAAAATTGATCTCAAAGAAAGAAAAGAGATAATATTTGG
TATATGGTGTGCCTCATTTCATGCCTGCCCTGCAACCTCTGCTACATTGCTGGGAACCTGTGGCCATCTCTGGAAGCAGTTTATAACAGAGTCT
GCAGAGAGTTAGCCTTTATAGCAAACTCAATGTTAAGATTGAACACAGCCGCTTTAGTGATTTAAGCATCACAGCATCTGCTGATCACTTGTTA
TCTTGTGGTTTTTCTTAGCCCCAGTCCAAGCCCCAACTGAATAAAATGGTAATCTTTCAGTTCTCAGTCTCTGAGCTCATCTCTGTAGACCCAT
GAGATAGACATAGGTACCTACTACTTTCGCCCATTCGATAACAGTGCGTGCCAGTTCAATCGAGTAACTAGGGTAAGCATAGCTTGGCTATAAG
GGAGTGTTTTTGTTCTTAGGAAGTAGATGCCAGGATGTTTGATGCTAACAAACATCCTATTTGAGTTACAATATATACCACAAATGGCTGAGAA
CTGATACTTAGGTATTTATGTAAATTTACACATGCAGAACAAGAATTTGGGTATAATATATATAGGTTGTCTTTCTACCTTTTCTCTTTTTTGA
AAGTTATTGTGAGTTTGAAATTATTTCTAAAAAATTTTAAAATAATAATTGGACTTGTTTTACGTTTTTAAAAACTTACAACTGTTTATGGAGA
ACAAGCAGCCTGAGCACGATCTTTTTGATCGACTTAATGTGAGTAAACACAGGACACTAGGCTAAAGTCCAAGCCAATCTCAAGCTCCTGGGGT
CAAGAGTTGTCAAAGGGTATTTGGACTGTCCATTCAGTCTAGATTGTCCAGTCACATTCTACATATCCCTCCTCCCTTGGGGCACTCAGTCATC
TGTAACCAGCAGCAGACCCTCCCTCCTCCCACCCTGTGGGTGGTGCTGTGGATTCCTACCAAAGCTGCAAGGCAGAGGGAGCTGAGCTTTCCAT
GGATCTTCCCTCTTCCTTTCATCCTTTCATGCTTTCATGAGACGCCCAGCTGCCTTTGCTACAGACCTAAGGCTTTCACTTGCTTTCTTTACTG
GGAGAGATATTTATAAGAAAGAAAAAATTTGTTGTCTTGAACTTAAGATTAAAGTACTTTATAGAAAGCCACTATGCAATCCTTCCTGTATTCT
GGAATTATTTTCCCTCTTTGCTAACTTCCTCTGTGAAGGACAGGACAGTAACTGGCAGGATGGTGGCAGCACTGGCACAAGGACTCCTAGTATT
CACTGTAAGGGAAAGGGTGATTTGGTGTTTACCTCAGTTCCAATATCCTCTTTGTTGTCGTGTTTAATGCAGACTGGTATTCTAAATAAACATC
TTCAGGATCTCATGGAGGGCTTGACTGCCAAGGTGTTCCGTACCTACAATGCCTCCATCACACTACAGCAGCAGCTTAAAGAGCTCACAGCCCG
TAAGTAATACTCTGTATGGTTGTCCTTGGGATGGTGCCAGTTTCCTTCTCTTCATTCTTTCCCGCCAACCTCTTGAAGAGGCTTTGCTGTCTAA
GCAAAACCAAGGCCTTTCTTCCCACTGCTGCTGCAAACTCTACATTAATCTCATAGAATACTAACAAGAGCTTTGCATCTGGTCTAAAAATTTT
TATGTTTTATCTGTGTTACTTACAAGCTCAGAGGGTATAGGCAGTGGTGTTAAATTGGGGAGAATAGAGTTGTCATGGTTACCACAAAAATGTA
GTAGTAAATCTTTTTCTTTTTTTCTTTTTGCTCTTCATAATAGTATGTAATAATTACCTCTGGGACCTTAGAACTTCTTTTCCTCTCCATTTAA
AACAAACTCTCACACTGTAGCTAAAGGCTGGGCACATTCATCATGTGATCCAAGCTAGTGAACCTTCTGCTTCAGCCTACCCACAGCTGACGCT
GTAGGCAACCCTTCTGCTCCTGCCACTTGAATTTCCACTGCCTTCTATAAATGTATCAAATGCTCTTAGACGAGCCAGAGATGGTATGCTGGAG
ATCAGTAATTAGGATTGGTTTAAACAGTGGCTAAAAAGCCTGGATGTGTTGGTGGCAGCCCAGCACTTGGAAGCAGAGGCAAACAAATCTCTTG
AGTTTGAAGCTAGCCTGGTCTATAGAGCAAGTTCAAGGACCCTGTCTTAAAAGAACACCCCATCCCCACTCCTACCCCTCCACAAAAAAGAAAG
AAAGTCAGATCCCTAGAACTAGAGTAATACATGAAACCAAACCCAGGTTCTCTGCATGAGCAGCAAGTATTTTTTAACCACTGAGCCTTCTCTG
CAGCCCCTGCACTATAGCTTAGGATCCCTTCATCTCATTCTCCTGTAGTCATTTCTTCTAGCACAGCTGCAGCATTTGCTACATCTGCCTAGTA
AGTGGGAGACAGGGAATAAGAAAAGCAGTATAAAGCTCTGGGGGCTTTGGGTTTTTGTTTTTTGTTTTGGTTTGTTTGTTTGTTTAAAGATTTA
CTTGTTTTATGTGAGTACACTGTCACTGTCTTCAGACATACCAGAAGAGGATATTGGATTCCATTACAGATGACTATGAACCACTGTGTGGTTG
CTGGGAATTGCACTCACAACCATCTCGAAAGCAGTCAGTGCTCTTAACAGCTGAGCCATCTCTCCAGGCCCAACTCTTGAAGTTTTTAAGAAAC
AAAACCAAAAATATAGATTTCCCTGTAATGTAGACAAGCTGGGCAGTAGTTATAGCTATTCAGGAACTTAGCAGTTTAGAGACCACCTGGACTT
CATAATGAGACCTGGACACAAAATCCAGTAAGGGTAAAGAAAAATATAAAAGAGAGGCTCAGAATTCCTCCTGTGACAGAAACTCATTCAGGTT
TATGTAGAAATTTACTGCCTTTGACTCAAATGTTGACCTTGAAACTTGGTTATGATAAACTGAAGGTTTGTAAAAACATCTAGAATGCATTTCA
TGTGTATATTCTTGGTATACAAACTGACCCAGTTACTATTATATTTCTTTGTAGCTGATGAGAATGTACCAGCGAAGATTCTATCTTATAACCG
TGCCAATCGAGCTGTTGCAATTCTTTGTAACCACCAGAGGGCGCCACCAAAGACCTTTGAGAAGTCAATGATGAACTTGCAGTCTAAGGTATGG
TGGGTCCAAACGTAGGGTAGCAAAGGGAGTCCATGTCTGATGTCTTCTGACAACCAGAGGCTTCTGGAATCTGACATAGTATGGCCTGGGATAA
CAGCAGGCTAATCTGGGTCGGTTTAGAAAGATTTTTTTTCAACAAATGGAAATAATTTGATAGTATCATTCTTTTTTTTTCCTAAAATATTTGT
GCTGGAGACACAAGTTCTCTATGAGCCACTTTCTGTTTTGAAGTGCTAACTTTGACATACCACTGAATTGCTAGTTGTTGTCCTTGTTATACTT
AGGGAAAATGGAAATCTTCGTGGTAGAATGCTTGCCTTGTGTGCAGCATGCTCTGGGCTCCATCCCTAGCATGACAAAAATAAAGTCTCGGTGA
TCTGGCTAAGAAACATGACAGAAAGATACACCTCACTGGCAGAACCCACCTTTGCTTGTCTACCATATGTGAAGCCCTGGGCTTGATCCCCAAA
GCAGAAGCAGGGGAAGGGAGAGGAATCCTGTTACATTGTTTGAAGTTTAAAAGATGGTGAAGCAATGGCATTTAATTCGCTCCTAATCCTAGAA
CAACTATTTACATCGGTTGAGGTGTGTCAGATATGACTGTGTCAGGTTGTGCCCTATTGGGAGAGACTGATACCAGAACAAGTTGTTGTTTCTC
TCTCGGCACTGGCTGCTTAACTATGCATTTAAATCCACAGTGAACATGTTAAAACAGACACACACCGTTACAATGGAAAACATTTAATACACAC
AGTCTGTGGCTTAGGAATAAGTATCCTCATTTAAGATCTTGAGGTGATCCTAACAATTATAAAGTTTAACACAATGATAATAAAAGTGACATTT
CCAGAGTTCTGCAACACTGCAATGCATAACACCAAAGTATTCACCATCTGTTGGCAATAGTCACAAGACAAGTTATTTCTTTGGATTCTTGCTC
ACAGTTAAAGACGAAATAATGTCAACTAGAGAGTAGCCAAAGTACAGATGCATTTTTTTTTTAATCCAAATTTATAGAATTCTATACTTGGACA
ATTTACTTTTAGAAACTGCTTTGCAATGAAACTTAACACATATTTTCAGGCTTAGAATATTCCCCTATTGAGATTTCTGCAAGATGAGAACACA
GGCATAATGTATATTAATCTAAAGCTACAACCTAGAGATTCTGATACCCCTGGCCTGAAAGCATCTCCAGATCCACAGGGAGTGAATCTCAAAT
CTGCACCTAAGGTCAGCAAAGGACGACGGTCAGCAAGCTAAGAACCCCACAGACTGCCTTTTGAGCATCAGCCTTTGCTTTGCCCTGTTAGATC
GAGATCCTCTGATGCTGCTTACTTTCCTCATGTGTTGTCATTCCAAGCCCAGAATAAATAAAGTATGAGTACCAGGTGCTATTTACACAGTTCA
GGAGACCCTTCCTGGAGAATGAGGAGGTCATTTGTTGCTCTCATTTGAAACCACTGTTTGAGCAGCTCCCGCTCTCTGCCAAAGAAGCCCTCCA
ACTGTAGGAACCTTCTCCAGTGTGCCCACAAAGTAAACACACGCCCAGTTGCCTAGTCAGTCAATGGAGTCTTTGAGAGAATTTGACATAGCTT
GCCAGTTTAGCATTCACAGAAATGGTTACGACTGTGATTTGCTGTGTTTCTAGTCTAAGAAGCCTCTGCCTAGTTGTGGCTCCAATGCTTTTGT
GTCCAAAGTCAGTTCTAGTACAACATACATAGTCGAAGGAAGCCACTAACAGTAAACATTTAATGATTCTCTCACAATGGTGAATTGTTTCTAA
TCTTGGGCTGCATTTTATGTGGGTCCCCTACCCCCACCCCCGGAAGGGTAGAGGCTGCCAATGGGTGTGGTGTTTTTCTAACAAATTTTAGGCT
CAGTCAACAACAACCCAATGTGTGTAAGTCAACATGTATTCACTCTGGGATATTCTCTTGACCTCATAGGTCAGCAGGGTGATTATGGGGATAG
GATTCTTTAGAATCCAGCAGCACAAACTCACCAGCATTAGCTACAATATCATCATATTGCTGTTTCTAAGCAACACCCAAAGCAGAATTTAGGT
TTTGTGGGTTCAACAAGCAGCAAAGAATGATGGAATTATTCTGTCTCTTCCTTGTAGATATGGATTCCATAAGATTTCCTCTGTGGAGGGAAAA
CTTCAATTGATGCTTATTGAATGATGACTAATTTTCTTCTCACTTTAGACTCCTTTACTTTCTAAAATAGTAAGCCAAGTTGAAGCAATTCCTC
ATTGATGTCTACTTGCCAGTTTATAATTTCCTGTGATATCCTCTTTACTATATCCTTTCCCAAAGCTGGCAATTCTTTCCCAGATTTGGCAGGC
TTGCTTACTAGATGTGTGTATCTCCTCTGCTACTCTCACAAGGAATGCTAAAGGCCCAGAGTGGTCGCCCACGCCTATAATCCCAGCATTTGAA
AGTCAAGGACAGGAAGATCACTCATCACTCAGTTTAAGGCCAGCTTAGTCTACAGACTGACTTCTAGTCTCTTCCTTCCTTCCTTTTTTTTTTT
TTGAAACAGGGTTTCTCTGTATAGCCCTGGCTGTCCTGGAACTCAACTCTGTATACCAGGCTGGCCTCAAACTCAGAAATCTGCCTGCCTCTGC
CTCCCAAGTGCTGGGATTAAAGCTGTGCACCACCGGCTCCAGTCTCTTTCTAACGAACTAACTAACTCATAAAACAAACAAAAGATGCTAAAGT
CTGGCATAGGATTAATTTGAAAATAGCTGTCATCTGGGTCCTCCTCTGTTTTCTAAAGCTAGTGTGTTTTCCTGAAAGAGTACAACCAGATTTC
CAGGTTACTCTACAGAACCTTATATTGTTCACCGAAGAACGTAAATGAGCAGCAGACAGCAGCTGGATTCTTTTCTGCAGTCACTGTCCACCAT
TTTCATAAAGCCTTTTTGTACTAGGAAGCACTACAGCTCCCAGTTTTACGTTCTCAGATGAAGCTTCTTTCCCTCTCCAGGTCATTTTATTCCT
AACTGCACTGCCGTTTTTCTCTGTCTATCTAGATTGATGCCAAGAAAGATCAGTTAGCAGATGCTCGAAGGGACCTGAAAAGTGCTAAGGCTGA
TGCCAAAGTCATGAAGGATGCAAAGACCAAGAAGTATCTACCTGACACTGTACCAAGGGTTGGATAAGGGAGAACAGTTGGCACCATCACTCAG
AGGCCTGAGTGTTCCAGCTCACATCGTCTCATTATGTAGCCTTGGCTTCCTAGGATAAACCACCATTCTCAGCTGTTTTCAAATACTTCTTGAG
GAAATTTTCCATGATTTTCTCCTTTAAGCAGTATATAGGATACTTGTTTGTTCTTGTCCAGAGGTCAGTCTTAATGTATGATTCTGCCATTCTT
CCAGCGTAGTAGAGTCAAAAAAGAACGCTGTACAGAGACTAGAAGAGCAGTTGATGAAGCTGGAAGTTCAAGCCACAGATCGAGAGGAGAACAA
ACAAATTGCCTTGGGGACCTCCAAACTCAATTATCTCGACCCTAGGATCACAGTCGCTTGGTAAGTCTTAGGCCTGTGTAGATTTCCTGCTTTT
GGTTAAGGAAAGGCAGAAAGTCTCTTGGATATTGCCAGCTCCTTCATGCCGTGTCTAAGTTTACATTTCTTCCAGGAGAGCCTACAGCTCTGGT
TCCTGAGGTACATGGAATATCCTAGGTGCCCCATGGGCACATTAGTATAGTAGCCCATGCCTGCTTTCGAGATCTCTGAGATAAAGAATGAGCT
GATAAAATAAAGTCCTTTGAATATTTTTGTGCACCAGAAAGCTCTGGACCCCTGCTATGGGTTATCATTGAGCTAGACTAATCTCCTTTGTTGT
CTCCATATTTATTCCTTTCTTTTTTCTTTCTTTCTTTCTTTCTTTTTTTTTTTTTACAGGTGCAAAAAGTGGGGGGTCCCAATTCACAAGATTT
ACAACAAAACCCAGAGGGAGAAGTTTGCTTGGGCCATTGATATGACTGATGAGGACTACGAGTTTTGACCAGTCTCGGCACGGCTTCTGTGAAA
AGGAACAGTGTGGTTTGGGGAAGATGGATAAACTGAGCCTCACTTGCCCTCACACCCGAGGGAGAGGCCACAAGTCTTAACAAACCAACATCTT
TCCAAAAAGATAGACCTGGAGATACTATAAGGGAGAGCTGAGCCAGTTGTCCTATGCACAACTTATTTAAAAATATTTCAGATATCAAAATTCT
AGCTGTATGATTTGTTTTGAATTTTGTTTTTACTTTCAAGAGAGCAAGTGGATGGGAATTTGTCAGCATCCTGCCAGGCAAATTCAGTTTTGCT
CCAATGTTTGGATTCTCTCAGCTACTGTATGTGAAGTCTGATTATATTGGTGCGTTTTTACAGTTAGGGTTTTGCAATAACTTCTATATTTTAA
TAGAAACAAATTCCTAAACTCCCTCCTCTCCCCTATTTCAGGAATTTAAAATTAAGTAGAACAAAACAAAAACCCAGCGCACCTATTACAGCTG
TCATTCTATCGTCATGGGAATCAGTTTTCATTAAACTTGAAGCAGTTGTGACTTCGGCAGTGTTTTGGTTCAGACACCTATTCACAGAAAAGCA
TGATCGCAAAATATTTCCTGACTTGACTGTTCCTTTTTAAATGTGAATTTTTATTTCGTTTTAATTATTTTAAAATATTTAAGCCTTCTTCTTG
ATCTTAAAGATCGTGTAGCTTGGGGTTGGCGACGGATGAAGGGCGAGTGACTCTAACCATAATGTAATAATCAGTGACTGAAACCATTTTCCCA
TCATCCTTTGTTCTGACCATTCTTTGTACCCTTTAAGAGATCCATCTTTTTCTTTGGAAACCCCAGTCTTTCATTTGAAAGATGTTATTGTATA
AAAACTTCCACACGTCAATAAATTTAGAGGAAAATCAGCATTTGGTCCAAGAAAAACAAAAACCAAAACAAAACAAAAAAGGAAAAATAATCAA
GATTTTAGGGCTTTTATTTTTTCTTTTGTAATTGTGTAAAAAATGGAAAAAAAAACATAAAAAGGAAAATTTTAATGTGAAGACTTTTTTTTTG
CTATAATCATTAGTTTTAGAGGCATTGTTAGTTAGTGTGTGTGCAGAGTCCATTCCCACATCTTTCCTCAAGTATCTTCTATTTTTATCATGAA
TTCCCTTTTAATCAACTGTACGTTATTTAAAATAAACTCCTACAACTTAACGGAAACTTAGCGTCTGCCTGTTTGTCAATGAGCCCCCACGCCA
GAGTAAAGGCTCAGGGCTGAGATTTAGTGCAGTTCATGGTCTATTCATTCAGGTCAGCAAGGGTCGGTCCCTCCCAGTGGGAAGGTGGTATGGG
CTTGTCCTGGGAGTACATCTGTCCTGATGTGGGATAAAGAGTATCAGTTTCCATCCCTAAAGACAGCCCCACAAAGCAAGGGAATGTACAATAA
ACCTTTAGGAACTGCCTAATCCCAGTGTGGGCAGTGCCTACTGAGTGGGACAGCCTTGTTTATTTGAGAAACAATCGTAGAGAGCTTTCCATGT
GTACTGAACAGAAACAGGATAGCTAGCTTATATGGAGAAGCCTCCAGACGGAATGAAGATTCTGAATTGATTGATTGAAGGACTTAACTGTTAA
CTGTAACAAGCCCAGAGCTCTGGATACCCCAAAGTAGCCATGATCCCTACCAAGTGTTCCTTCTGTTACCCTAAGTCTGAAATTCAAGGTGAGA
AAGTGAACTCCAGACTAAAGGGAAGGTCCGAATGGGCCACCAATTGAAGGGCCCACACCTGAGCTTGCTGGCATCCGCAATCTATTCAGAACCG
CCTTTCTTAGTGGTCTGCGTTATCACTGATGCTGGGACCACAGCAGCAAACTCTTGGTCCCTTGAAGCTTGAACTTCAATTGGCTATTGAAGGC
AGCACTTACTCCTGAGAATAAAAACAGTATAACCATCACTGTTTGGAACCAACAACATTATAGCCAGGTGTTTTTGAATTCTGTAGTGTTAGAG
TTAGCCAGACAGAATGTTAAAACGGGAGGCCTTTGGTAACCAACCAATACTCTCTGGCTGGTAGCTGGTAAGAAATGAACCTTAGTTTTAGGAC
CTGGTAACAAACACAACAGCAAATAGCAAAGTGCATTCCAGGCAGTCGTGGCGGGCCTCTTGAGGAAAGCCACAGAGCACACATGAATCTCAGC
TGATCCTGACTGGGACTGAGAAGAGGCTGCCAAGATTGGGGGAGCTGATGAGTTTTAGAGGTATATAAGGCTTGCGCTTAGAGAATGGGAGTAA
ACCTGCCATCCTTGATGACCCAGCACATCTCAATTGTAGCCCATTATGCAAGAGCAGAAGCTAGGTGGGCAGTATCTGGGAAGCCCCTTTCTGT
GGGCAAAGGGCAGTGCTTCTATGTGGCACCAAGCTACACATCATCCACACTCTTGCCATCTGTGCAGCAAGTGCACTGTCTCTTCCAAAAGCTG
GGACCTAACAACTGACACATTTAAAAAGTGAAAAGAGACACTTCACACACACACACACACACACACACACACACACACACACACACACACCTCA
GCCTTTCCAGCCACAACCTCTCTTCAGTTTCACAATCAAGTAAGTACCTGCGATGAGTCACCCACTGCACTGTCTTTTCCCACCCTCTGCATCC
AGTGTCTACCACAACCCTCTCCCTGGGTTCTAACCCTGGTCACCAGTGACCACGCAACAAGGAGAGAGTGCAGTCTTCATTGACCATCTTGCCT
AAGCTCTTTTTGGGTGGAATTGAATGCTGTTACTTTACATGCCATAAATGTAACATTTTAATAAATGATTTTTAGTAACTGCTGACAAGTTCAT
CCATCATCATGGGTCAGTTTTAGATCACTTTTCATCCCTCACACCCATTTGCAGCTATTCTGCACTCCCATATGCATCCTCCACTGGTCTCAGG
TTTATTCTCTAAACATGTCTTATGGATGTTTTATTTGAAAGAGCTCATAGTATATGTCCACCCCTTGTAAAAGTGCCTCTCTGGCTCCTGTAAT
GTCGCCCAGTTTCACTCCTATTTCTGTCAGGCTGTTTGTGGCTGAGACATTTTTAATGTAGCTGCCATCGTCCTTCTCATTTCATGTAGCCAAT
GCAGTCACACTCCTGACCTCTGCACGCACAGTCAGCCACACAGTTCTGCAGGATATCCACAGAAACCTCTGGACGACAACTCAGGACTCGCTGT
TCTAAGCCTGCTGGGTCTGGGTTTCCTGTGTGTGAAAAGCACCTCACTGAACCAGGCAGACTCAGCCCTTGGCCCTTCCTTCCAAGCTCATATC
TAGTAATTTGTCCACTTTGAAATTCTTTCCTAAATACTTGTACCACATATAGCCATTTTCCCAATTTTTAAAATTTCTTACATTTTTTTAAACC
TTTTTTTTTTCTAGAACTTTTATTTCAGCCCTTGCAGATCCCTCCTATAAGACATTTTCAATTTGTTCGGAACAAAAGAGCACTTCTTGGAGAA
CATACTGCCCTGAAATGACCTCAGTCAAGCTAGTTTTGGATGCTTATGAGTAGTCCCCTGATGACAAATCAGTAAACAGGGAGGGAGGGGAGAT
TTAACAGACTTTCCCCACAACCCTAAGGAAGCAGCCTTTTTGAAATTGCCATATAGATTTTTTAGCTTAAATAATTACGCTGTACTTACATATT
TTAAAAAATCTTTCAGAGAAAATATTCCTGGAAAACAGTGCTCATCAGTGCAGAGGCTGGGGCTAGTGGTGTAGGCCAGAGGCAGGGCCCGTGA
CCCTGGGGTCTGCCCATAACACTATAAAACCACCACCACCAAAACCGTCAATGTAATCAGTTCCCAAAGTTGGGTGGAGGCTCACTATACTAAC
TGTCATGCCTGTACTTACATTGGAAAATCCTTATAGCCGGGCAGTGGTCGCGCACGCCTTTGATCCCAGCCCTTGGGAGGCACAGCCAGGCAGA
CCTCTGAGTTCGACGCCAGCCTGCATGTTTAAATTAAGGCAATTATGCTTAAATTAAAATTCCCAGTCACCGCAGATCTGCCCTATATACTGTC
TGGGTAGAAGTTACAAGCAGTATATTTTTCAAACTCAAACTTCACACGTAAGATGGGTGCATCTTAGGGCTAGAGAGATGCTTAAGGGGTTAAG
AACACTTGTTACTCTTGCAGAGGATTGGTGTTTGGAACCTAACAACTACATGGTGGCTCACAGCCAACCAAGTAACAATTCCAGGTGATCCAGT
AATCTCTCCTGACCACTGTGCACCGGGCACACAACTGGTGTGCATGCGTATACACAGGCAAAGCACTCATACACAAAGTAATTTTTAAAAGGTA
GGTACAGCTTACGTGTAAATTATTCTTCAATAAGTCTGGGCTTTCAGAAGCAGTGGGGACGATGTGCTTTGATAACATTGGATGGTTCTCTCAT
GGTGAAAAACACAGACTCAAGGCTGGAGAGATGGCTCAGCAGATAAGAGTCCTGAATTCCCAGCAACCACATGGTGGCTCACAACCACCTGTAA
TAATATCTGATGCCCTCTTCTGGTGTGCCTCAAGACAGCAACAGTGTACTCATCTATAATAAATGAATAAGTCTTTAAAAAGAGAAAAAGACAG
ACTCAAAGATTCATGCAAAGTTTACTGATAGGACTGAATGTAGGGAATCAATATTTATAAATAGTTTTTAACAATCCTATTCAAAGTATACACG
TGCCACTGAACACATACTTGGCATTTTAATCATTCATCTTTAAACCTTTGTACTTCCTTTGCAGAAACACTCCACAAAATACCTTGGGGTGACC
TTATTGTGAAAGGAATAGTTACCTCATTTGGAGAGGCTGTTTGTCATGAATACACAGCACCTCTCACTTTGTTCTCTTCCCATAATCACTAAAG
AATCTAGATAAGGTCCTTCTTGCTCCCTGTGCCTTTGTCAGACCAACCATGGCCTGTTTCTGAACAGGCTTGCTGTGTTCCTGCCCTGCTGAGA
TTCTCTAGAAGGCACAGTAGGGATGTGTGCTGGAACAAAGGGGGCGAGAGTAAAAGCAAGTCCATGCTGGTCTCAGTTTCCCCTGTCTTCAGCA
CCACATGCTTGACCTCAGTGAAACAAGCACTGTACCGGCCGTTCCTGGCTGAAGAGAGCTAATGGTAACAGTCAGTAGTGAGGCAGCCAGGGCT
GTGGACACAAGAGGTTGTTGGGAACTGTTGGCATAGATGTGAGTTATGGAGCTTTTGTTCTACAAGACAGGGTTTCTCTGTGTACCCTTGGCTA
TCCTGGAACTTGCTCTGCAGACCAGGCTGGCCTCAAACTCATAAAGATCTGCCTGCCTCTGCCTCCCAAGTGCTGGGATTAAAGGTGTGCCACC
ACCACCCAGCAGGTACGATTTTTAACTTGAGTAAAAACAAACAAAACCCCTAAAGTTCCTTTAAGGGACTGAGAATGCAACTGACTTAAGGAAA
GGAGCCCCGAGGTTATGATGTCAGAAGGGATGGGGCCCTAGATAGGTTTGCAGGTCAGAGGACAAGGGCTTATACTGCAGGTTCACTGAGGTGT
GCACTTAGGAAGTGCTTGCTGCATGTACAGTTAACAGCAGAGGAAATAAGGGACGGAACACAGCTTGATGAATGGATTAAGCAGACACTTCAAG
TCTAAGTCTTTGTCTCCCTCTGTGAACGGAACATCACTATGCTCTGACATGATGACTTCTAGAGTCAATCATTATAACGAAGAAAAGTGTGTTA
GCACTTGGTACTAGCCTGCTAAGAATGCCCTCTGTTATTTTAATCACATACATAGTCATGTGAGAATATTTTCTTATAATTTAATTCCAGGAAA
ATGTTATTCCCTTTCCACAGAACTAGTTAGCAAACATTGAGAGTCACTACTTTAGCTACTATAGAGCTCAGCCACAGCTGCCTCTTAATCGCAG
CAGCTCCATTACCACTCAACCATACTGTGATTGCCCTTATAATCACATCTCCCATTTCAACCACACAGCATTTTTGTCCTTTTCTGTTTTAAGT
GCCCTCTATTTTTCCTAAACAGCTTGGTGCAGTGGTGCCCACTGGATGTCTCAATTCTTGGGAGGCTGGCCAGGCCTCAAGTTTAAGGCCAGCC
TGGGTTCTACAAGCAAGACCATACCTGAAGAAAAATGCAGTGAACATAGCAGCGTACCTGCTTCCTGTTCAAGGAAAGACTTGTCATCACTTCT
GGAAGAAGTGTAAATAGAAATCCATCTGCCAGTCCGTCAGCTCACAAAGAGCCAGGAGGCTGAGGATGAACGTTTTGCAGAACAATCAACATCC
ATTCCAGGATAGCACTGCACAACACAGTTCCCTGGAAGTTGTCTGAGGCTGTTCGCACTCCCATCTCCTTCCTGTTGCTTCCACAATGCTGACC
TTAGGGCTGCTCCCTAAAAAGTATTCTACCCACTAACCTATCAGAGACAGCACCCTAAAACAAATTCCGACCTAGCCGGGAAGCACAGTAGCTC
ACATTTTTAATCCCAGCAAGGCTGAGTTGCTGAGTCAGGAGAATAGCTAAACTCAAGATGAGCCTGGATTTGTACAAACTTGTACAAAATAACT
AANNNNNNNNNNNNNNNNNNNNNNNNNNNNNNNNNNNNNNNNNNNNNNNNNNNNNATCTCTAGGACCCACATGGTAACTGTAAATAAAGAAGCT
CACTTAGGATACCCAAGATATAAGATACAATTTGCTAACGCATGAAATTCAAGAAGAACGAAAGACCAAAGTGTGGACACTTTGCCCCTTCTTA
GAAATGGGAACAAAACACTCATGGAAGGAGTTACAGAGACAAAATTTGGAGCTGCGACGAAAGGATGGACCATCTAGTGATTGCCATATGCAGG
GACCCATCCCATAATCAGCTTCCAAACGTTGACACCATTGCATACACTAGCAAGATTTTGCTGAAAGGACCCAGATATAGCTGTCTCTTGTGAG
ACTATGCCGGGGCCTAGCAAACACAGAAGTGGATGATCACAGTCAGCTATTGGATGGATCACACGGCCCCCAATGGAGGAGCTAGAGAAAGTAC
CCAAGGAGCTAAAGGGATCTGCAACCCTATAGGTGGAACAACAATATGAACTAACCAGTACCCCGGAGCTCTTGTCTCTAGCTGCATATGTATC
AAAAGATCGCCTAGTCAGCCATCACTGCAAAGAGAGGCCCATTGGACTTGCAAACTTTATATGCCCCAGTACAGGGGAACCCCAGGGCCAAAAA
GGGGGAGTGGGTGTGTAGGGGATTGGGGGGTGGGTATGGGGGACTTTTGGGATAGCATTGAAAATGTAAACGAGGAAAATACCTAATAAAAAAT
AAATTAAAAAAAAGAAGAAGCTCACAATATGCTTACCACTGTTCTAAGTAGTTATTAAACCCAATATGAGATCAGGCCCACAGGCCAGAGGGGC
AGCTGGGCAGTCTTTCCTTGACTCCAGGAACAGGCTTGGTATAAGCTAAGGTTGAAGCATGAGTGTTGCCTGAAATAAGCCCCAATCTGGGCTA
GTCGGGGTGACGTCACAGGCAAGTGCCTTGTGTGGGGGCTAGAGCACCTCCCAGACAGCACAAATGTATATACATTACCAGTGACACCTACACA
TCCTTATATATGCTGAGCTGGGGGAAGCCCATACTGGCTACACCCTGTGTGTGCCCTACTTTTCCCAAACTTCCTTGGGTAGAAGGAGACACCT
AAGGCCTAGGAAGCTAGGAAGAGGAAATGAGGCCCTGTTTCCCAATTATAGGCCTCGCAAACAAATTGCTCTTGGAAGAAAATCATGGAGCCCC
TGGGGGTGGGGGTGGGGTCTTGTTACAGACACAAAAGCAAAAAAGGTTTGGGCTGAACACAAGGAAGCTCCCAGAAAATAGAATTTTAAAGCAT
ACTGTAGCCTCTAGGCTGCTCGAGTTGGCACAGAAAGCTCAACAACGATTACAACATTTTAAGAGGGTGTCTCCTGAGAGAACCGGCCAGACCA
GGCAGGCAGGGAAAGGCCCTGTTCAGCTCCATCTGCCCTCTCTGTCTAATCATCCTCGCTGGCCATTCCCCTCCACTGAGTAAAAACCCCAGGG
CCTTTGCACATGCTGTTATTCATACCTGGAAAGTACTTTCCTCCAGCCTTCCCTTCAAAATACATATGTCCATCTCTTTACACCATTTGGCCTC
TCTTGAAGTCCTACAGACATAAGACACAGCTCCTCTTACTTAATGATTGGCCAGCTAGCCAATGGCCACCAGGATTACCTCTCACCCAGTGGGC
TCTCTTGTGGAACACAGACACTCAGCTTCCCACTTATTACATAGCCTGACTTCAAGTCACGGGGCTCTGAAACCTTGATTAGAAGATAATTAGG
ATCATGAGTTGTGCAAGCCAACCTCAAGTCCAGTTTCCGCCTACATCAGAGGCCTGTCACTAGGTGGCAGTCAGTCTATCTCAACTCCCCAGTC
CCTCAGGACAAAGTTCCTCAGTGGTATACAGAATTCGGACGTTCTTGAGACCACCCTCAAGGGAAATAGCGCATCTGTCACTATTTGCCTGGCA
AAGCGAAGGTGGCCCAGGTGGGTGGGTTGTCCAAACCCATACATCACAAATTCTAGTCTCAGGAATCCCAGGGGAGCTTTCCTGCAAGGGGATG
TTGTGTGGGACTTCTCACCTCTGAAATGCAGATGCTGTCCAGAGCTGCAGGGTAGCTGTCCAACTAACTGTCTGCTCATCATTGGCCAGACGGC
AGGATCAAGGGATGCCCTCGGGTTTACCCGTGCTGTTCAGAGAACGCCTTTTGGAAGATTGATTTTAAGTTATTTAACAGTCACAGACAGGTGT
CATGTCTGGAGAATAGAGGCAAAGTCCGCGGTGAGGGATGAAGCAGGAGAGATAGGGGAAGGCAAGACAGGACTGCTGGGCCACGAAGCTGTGC
TGATTTGAAGCACAGTGGGAATTCACGTACGCAATTTCAAAGGCTTTAGTGGTAAATTCTGAAGCTCAGATGCAGGCAAGACCCAAGAGGATAG
TGTACACAGAGAGAAGAGGGGTCCTCAGGATCGTGCGTAGAGTGGAGAGAGCCCCAAAGGCAGGAGGGAAGAGCCTCAGTGGATTACTTAGGGA
TGAGGGAGAGAAGAAAAAACGTTCTTGCAAGGTGTGGGGTCTTCCAAATTCAGGAGTTCACTGCCATATAGAGAAGGTGTAGCGGGTGAAAGGG
GCCATGTGATGAGGATGGCAAGCAAGGCTGTGGCGCAGATGACGAGATGCCTGGGTCGGGAGGTCAGGGGAGACCCAGGATTGGGGTCACCTGT
GTCTGCGCAGAGGGGAAGCCACCCTGCAACTGGCCCAGCACTGAGTCCAGAGGAAAATGAGGCAGAGGACAAACCAGAGCTTCGGAGACTAAGT
GCAGGTAGGGCGCGGGCGGAGCGTGACGAGGGCAGCGGACCACGCCACAGGCCTCGAAGGCCACCGGACCCGCGTCCGAGAGTCTGAGGGCCCT
GCCCACACCTGCGTGGCCCCCTCCCCAGAGGCCACACTCCAAGGCCACCCTAGAACCCGTCTGTCTGCTCAAGCCCTTGCAAAAGACGTCTGCG
CAGAGGGGGCCTGGCAGGCGTGCTGTCACTCACGGCCTGTTAGCCAATCCACGAGTGCGCCCCTCCCCGGAGAGGGTGCGCGGAGGGCCCGCCC
CCGCCGCCACCGCGGGTGTGAGGAGGCCAGGCTGGCGCGGCTCCCTCCGCCCGGCAGCCTTGCCAGGTAACCGGGTTCGGCGGGAGGGCTGGGG
GTCGCGCAGCCCCCTCGCTCCCTGGGAGGCGTGCACACTGCCGCGGCGGGTCCCGTGTGGGCCGGAGGCCCGTGCNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCC
TCGCGCCTGCCCGCCTCCCAAACACTTGGGCAAACTTTCGGGCCTCGCGCCTGGCGCCCCGTCTCCGCCCAGTCCCTGGTGGTCACTCTGGGGC
GGGTGGAGGGGGGCATCCGGGTCTTGGATCACCTGATAGGACACCCCCTCCCCCAGTAGGGGGGGAGTGTTCCAGGCACTTTGCCCTGAGGCCT
AAGAGTCCTCACTGGTTGGACAAGTGGAGTGGGATTCCGGCCCTTAGCATCGGGCGGCTGTCAGTGGCTGTCAGGGGAAGCCAAGACAGGGACC
CCCTCATCCAACCTGAGAACCTGGGGAACCGACAAGATCTTCCTGCCCACTGCCATTTCTCCAGAGTGTGCTGTCTGTGAAAACTCCTAAGAGC
TCCGGGATGGGCTTATTGGCGCAAGAACCTTTGGAATCCTCATGTAGAACTTAGGCAGATGTTGGGGTAGGGCTGGTTGTGGAGCAGAGCGGGC
CTACTCATCTCCCTCTTCTTTGGGAGGATGGGGTATGAAAGCTAAAACCGTGACTGCTTCCCCCTCCCCATGTCCCGTGGATGGTTTTTTTTTT
TTTTTTTTTTTTGCCCCAGATCTGAATTTTGGAGGTCCA
MOUSE SEQUENCE—mRNA
ATGAGTGGGGACCATCTCCACAACGATTCCCAGATCGAAGCGGATTTCCGATTGAATGATTCTCACAAACACAAAGATAAACACAAAGATCGAG
AACACCGGCATAAGGAGCACAAGAAGGATAAGGATAAGGACCGGGAAAAGTCTAAGCATAGCAACAGTGAACATAAAGATTCTGAAAAGAAACA
CAAAGAGAAAGAGAAAACCAAACACAAAGATGGCAGCTCAGAAAAACATAAAGACAAACATAAAGACAGAGACAAGGAAAGACCGAAGGAGGAA
AAGATTAGAGCTGCTGGGGATGCAAAAATAAAGAAGGAAAAGGAAAATGGCTTCTCTAGTCCGCCACGAATTAAAGATGAACCTGAGGATGATG
GCTATTTTGTTCCTCCAAAAGAGGATATTAAGCCATTGAAGAGGCTTCGAGATGAAGATGATGCTGATTATAAACCTAAGAAAATTAAGACAGA
AGATATCAAGGAGAAGAAAAGAAAACTAGAAGAGGAAGAGGATGGTAAATTAAAAAAGCCCAAGAATAAAGATAAAGATAAAAAAGTTGCTGAG
CCAGATAATAAGAAAAAGAAGCCGAAAAAGGAAGAGGAACAGAAGTGGAAATGGTGGGAAGAAGAACGTTATCCAGAAGGCATCAAATGGAAAT
TCCTAGACCATAAAGGGCCTGTCTTCGCTCCACCATATGAGCCTCTGCCAGAGACTGTCAAGTTTTACTATGATCGTAAAGTTATCAAGCTGAG
TCCTAAAGCAGAAGAAGTAGCTACATTCTTTGCAAAAATGCTTGACCACGAATATACTACTAAGGAAATATTCTGGAAAAATTTCTTTAAAGAT
TGGAGAAAGGAAATGACTAATGACGAAAAAAATACGATTACCAACCTAAGCAAATGCGACTTTACACAGATGAGCCAGTATTTCAAAGCGCAGT
CAGAAGCTCGGAAACAGATGAGCAAGGAAGAAAAATTGAAAATCAAAGAAGAAAATGAAAAGTTATTGAAAGAATACGGATTTTGTGTAATGGA
TAATCATAGAGAGCGGATTGCCAACTTCAAGATAGAGCCTCCGGGGCTTTTCCGAGGCCGAGGGAACCACCCCAAGATGGGTATGCTGAAGAGA
AGGATCATGCCTGAGGACATCATCATCAACTGTAGCAAAGACGCAAAGGTTCCTTCTCCCCCTCCTGGTCATAAATGGAAAGAAGTCCGACATG
ATAACAAGGTTACTTGGCTGGTCTCCTGGACAGAGAATATCCAAGGTTCTATCAAATATATCATGCTGAATCCCAGTTCACGAATCAAGGGTGA
GAAAGACTGGCAGAAGTATGAGACTGCGCGGCGTCTGAAGAAGTGTGTGGACAAGATCCGAAATCAGTATCGGGAAGACTGGAAGTCCAAAGAG
ATGAAGGTTCGTCAGAGAGCTGTAGCACTGTACTTCATTGATAAGCTTGCTCTGAGAGCAGGCAATGAGAAGGAAGAAGGAGAGACGGCAGACA
CTCTGGGCTGCTGTTCACTTAGAGTGGAACACATCAATTTACACCCAGAATTGGATGTTCAGGAGTATGTGGTGGAATTTGACTTTCCTTGGAA
GGACTCAATCAGATACTATAACAAAGTCCCAGTTGAGAAACGAGTTTTTAAAAACTTACAACTGTTTATGGAGAACAAGCAGCCTGAGGACGAT
CTTTTTGATCGACTTAATACTGGTATTCTAAATAAACATCTTCAGGATCTCATGGAGGGCTTGACTGCGAAGGTGTTCCGTACCTACAATGCCT
CCATCACACTACAGCAGCAGCTTAAAGAGCTCACAGCCCCTGATGAGAATGTACCAGCGAAGATTCTATCTTATAACCGTGCCAATCGAGCTGT
TGCAATTCTTTGTAACCACCAGAGGGCGCCACCAAAGACCTTTGAGAAGTCAATGATGAACTTGCAGTCTAAGATTGATGCCAAGAAAGATCAG
TTAGCAGATGCTCGAAGGGACCTGAAAAGTGCTAAGGCTGATGCCAAAGTCATGAAGGATGCAAAGACCAAGAAGGTAGTAGAGTCAAAAAAGA
AGGCTGTACAGAGACTAGAAGAGCAGTTGATGAAGCTGGAAGTTCAAGCCACAGATCGAGAGGAGAACAAACAAATTGCCTTGGGGACCTCCAA
ACTCAATTATCTGGACCCTAGGATCACAGTGGCTTGGTGCAAAAAGTGGGGGGTCCCAATTGAGAAGATTTACAACAAAACCCAGAGGGAGAAG
TTTGCTTGGGCCATTGATATGACTTATGAGGACTACGAGTTTTGACCAGTCTCGGCAGGGGTTCTGTGAAAAGGAACAGTGTGGTTTGGGGAAG
ATGGATAAACTGAGCCTCACTTGCCCTCACACCCGAGGGAGAGGCCACAAGTCTTAACAAACCAACATCTTTGCAAAAAGATAGACCTGGAGAT
ACTATAAGGGAGAGCTGAGCCAGTTGTCCTATGGACAACTTATTTAAAAATATTTCAGATATCAAAATTCTAGCTGTATGATTTGTTTTGAATT
TTGTTTTTACTTTCAAGAGAGCAAGTGGATGGGAATTTGTCAGCATCCTGCCAGGCAAATTCAGTTTTGCTGCAATGTTTGGATTCTCTCAGCT
ACTGATATGTGAAGTCTGATTATATTGGTGCGTTTTTACAGTTAGGGTTTTGCAATAACTTCTATATTTTAATAGAAACAAATTCCTAAACTCC
CTCCTCTCCCCTATTTCAGGAATTTAAAATTAAGTAGAACAAAACAAAAACCCAGCGCACCTATTACAGCTGTCATTCTATCGTTATGGGAATC
AGTTTTCATTAAACTTGAAGCAGTTGTGACATTGGCAGTGTTTTGGTTCAGACACCTATTCACAGAAAAGCATGATGGGAAAATATTTCCTGAC
TTGAGTGTTCCTTTTTAAATGTGAATTTTTATTTCGTTTTAATTATTTTAAAATATTTAAGCCTTCTTCTTGATCTTAAAGATCGTGTAGCTTG
GGGTTGGGGAGGGATGAAGGGCGAGTGAGTCTAAGGATAATGTAATAATCAGTGACTGAAACCATTTTCCCATCATCCTTTGTTCTGACCATTC
TTTGTACCCTTTAAGAGATCCATCTTTTCTTTGGAAACCCCAGTCTTTCATTTGAAAGATGTTATTGTATAAAAACTTCCACAGGTCAATAAAT
TTAGAGGAAAATGAGCATTTGGTCCAAGAAAAACAAAAACCAAAACAAAACAAAAAAGGAAAAATAATCAAGATTTTAGGGCTTTTATTTTTTC
TTTTGTAATTGTGTAAAAAATGGAAAAAAAAACATAAAAAGGAAAATTTTAATGTGAAGACTTTTTTTTTGCTATAATCATTAGTTTTAGAGGC
ATTGTTAGTTAGTGTGTGTGCAGAGTCCATTCCCACATCTTTCCTCAAGTATCTTCTATTTTTATCATGAATTC
MOUSE SEQUENCE—CODING
ATGAGTGGGGACCATCTCCACAACGATTCCCAGATCGAAGCGGATTTCCGATTGAATGATTCTCACAAACACAAAGATAAACACAAAGATCGAG
AACACCGGCATAAGGAGCACAAGAAGGATAAGGATAAGGACCGGGAAAAGTCTAAGCATAGCAACAGTGAACATAAAGATTCTGAAAAGAAACA
CAAAGAGAAAGAGAAAACCAAACACAAAGATGGCAGCTCAGAAAAACATAAAGACAAACATAAAGACAGAGACAAGGAAAGACCGAAGGAGGAA
AAGATTAGAGCTGCTGGGGATGCAAAAATAAAGAAGGAAAAGGAAAATGGCTTCTCTAGTCCGCCACGAATTAAAGATGAACCTGAGGATGATG
GCTATTTTGTTCCTCCAAAAGAGGATATTAAGCCATTGAAGAGGCTTCGAGATGAAGATGATGCTGATTATAAACCTAAGAAAATTAAGACAGA
AGATATCAAGGAGAAGAAAAGAAAACTAGAAGAGGAAGAGGATGGTAAATTAAAAAAGCCCAAGAATAAAGATAAAGATAAAAAAGTTGCTGAG
CCAGATAATAAGAAAAAGAAGCCGAAAAAGGAAGAGGAACAGAAGTGGAAATGGTGGGAAGAAGAACGTTATCCAGAAGGCATCAAATGGAAAT
TCCTAGAGCATAAAGGGCCTGTCTTCGCTCCACCATATGAGCCTCTGCCAGAGAGTGTCAAGTTTTACTATGATGGTAAAGTTATGAAGCTGAG
TCCTAAAGCAGAAGAAGTAGCTACATTCTTTGCAAAAATGCTTGACCACGAATATACTACTAAGGAAATATTCTGGAAAAATTTCTTTAAAGAT
TGGAGAAAGGAAATGACTAATGACGAAAAAAATACGATTACCAACCTAAGCAAATGCGACTTTACACAGATGAGCCAGTATTTCAAAGCGCAGT
CAGAAGCTCGGAAACAGATGAGCAAGGAAGAAAAATTGAAAATCAAAGAAGAAAATGAAAAGTTATTGAAAGAATACGGATTTTGTGTAATGGA
TAATCATAGAGAGCGGATTGCCAACTTCAAGATAGAGCCTCCGGGGCTTTTCCGAGGCCGAGGGAACCACCCCAAGATGGGTATGCTGAAGAGA
AGGATCATGCCTGAGGACATCATCATCAACTGTAGCAAAGACGCAAAGGTTCCTTCTCCCCCTCCTGGTCATAAATGGAAAGAAGTCCGACATG
ATAACAAGGTTACTTGGCTGGTCTCCTGGACAGAGAATATCCAAGGTTCTATCAAATATATCATGCTGAATCCCAGTTCACGAATCAAGGGTGA
GAAAGACTGGCAGAAGTATGAGACTGCGCGGCGTCTGAAGAAGTGTGTGGACAAGATCCGAAATCAGTATCGGGAAGACTGGAAGTCCAAAGAG
ATGAAGGTTCGTCAGAGAGCTGTAGCACTGTACTTCATTGATAAGCTTGCTCTGAGAGCAGGCAATGAGAAGGAAGAAGGAGAGACGGCAGACA
CTGTGGGCTGCTGTTCACTTAGAGTGGAACACATCAATTTACACCCAGAATTGGATGTTCAGGAGTATGTGGTGGAATTTGACTTTCCTTGGAA
GGACTCAATCAGATACTATAACAAAGTCCCAGTTGAGAAACGAGTTTTTAAAAACTTACAACTGTTTATGGAGAACAAGCAGCCTGAGGACGAT
CTTTTTGATCGACTTAATACTGGTATTCTAAATAAACATCTTCACCATCTCATGGAGGGCTTGACTGCGAAGGTGTTCCGTACCTACAATGCCT
CCATCACACTACAGCAGCAGCTTAAAGAGCTCACAGCCCCTGATGAGAATGTACCAGCGAAGATTCTATCTTATAACCGTGCCAATCGAGCTGT
TGCAATTCTTTGTAACCACCAGAGGGCGCCACCAAACACCTTTCAGAAGTCAATGATGAACTTGCAGTCTAAGATTGATGCCAAGAAAGATCAG
TTAGCAGATGCTCGAAGGGACCTGAAAAGTGCTAAGGCTGATGCCAAAGTCATGAAGGATGCAAAGACCAAGAAGGTAGTAGAGTCAAAAAAGA
AGGCTCTACAGAGACTAGAAGAGCAGTTGATGAAGCTGGAAGTTCAAGCCACAGATCGACAGGAGAACAAACAAATTGCCTTGGGGACCTCCAA
ACTCAATTATCTGGACCCTAGGATCACAGTGGCTTGGTGCAAAAAGTGGGGGGTCCCAATTGAGAAGATTTACAACAAAACCCAGAGGGAGAAG
TTTGCTTGGGCCATTGATATGACTTATGAGGACTACGAGTTTTGA
HUMAN SEQUENCE—GENOMIC
TTCAAACTCCTGGCCTCACGTAATCCCCCCACTTCAGTCTCCTGCCTAGCTAGGACTACATGTCCATACTCCCATCCCTGACTAATTTCAAAAA
CATTTTTGCGGGGAGACATGAGGTCTTCCTATCTTCCCTTGTCTGGTCTTGAACTCCTGGGCTCAAGCAATCCTTCCACCTTGGTCTCCCAAAG
CATTGAGATTACAGGTGTGACCCACCATGCCCAGCCAAGCCTCAAGTCTTGAATACTTTAAATCCATTTTCTTTCTTTTTTTTTTTTTTTTGAC
ACCCTCTTGCTCTGTTGCCCAGGCTGCAGTCCAATCGTCCAATCTCAGCTCACTGCAACCTCTGCCTCCCAGGTTTAAGCAATTATACGTGTGT
CCCACCACACCCAGCTAATTTATCTCTGTCTGTGTGTGTGTGTGTGTGTGTGTGTGTGTGTGTCTATTTTTACTACACACCGCATTTCCCCAAC
TTCCCCAGGCTGGTTTCACACTCCTCAGCTCAGCCAATCTGCCCACCTCAGCCTCCCAAGGTGCTACCATTACAGCCATGAGCCACCATGCCTC
CCCTGTAGGTAATATTTTCTAAAACACATATTATGTTCCTCCATTGCTCATAGCAACTCTCAGTGACTTCCCACTCCTATCAACATAAACTCCA
AATGCCTTAGAGACCCTGCATGAACTCCCCCTTGCTTACCTCTCCAGCTTCATCTCTCACTACTCCTCTTCACCCTCTACCCCTTACCCATACT
GAATTACCAGCAGTTCCCCACATAAACCCTGCTCTTTCCAGTTTCCAAATTTTTCCCCATCTCTTCTCTTGGCCTTGAGTGCTTGTCTCCCTCA
CTTGCCTTCTTTATTCTCCCCAACCTTTACCTGGCTGACTTCTGTTTCCATTTTGAGACCTAAGTTTAAGTGCCATTTCCACTGGAAATCCGTG
CCTCACTCTCTAATTCTGGATGGCAGTTCTTCTAAACGCCTCCTACCCCAAACACACTCTACTTTATCCTCGACGCACTCATCACAGTAAGTGA
CAATTACTTGTTCCCTTCTCTGTCTCACCCACCAGACCTGACCTCCTTGAGAGCAGGGGCCTTGTCTTTCTAGTTCACGGCTGAATTCCTAATA
CTAGCTTAGTACCTGGCTTATAGTTGATTGTGTGTGTGTGTGTGTGTGTGTGTGTGTGTGTGTGTGTGTGTGTGATGCAGGGAAGCCAAAGAAG
GGTCAGGATGCAATGATAACCTGGGGAAGGAGGGACAATTACAAAGGAAAGTCTTGGGCACATTCCATACACGTAGTAGCCACCAATGGGATGT
GGAGGACAGGATCAAGGGTTAACTCAAGAGGCTTCAAGGTTCTGAGCCTGAGTGACTGGGAGGAATAGAGCTATTCACTAAGCAGAGACCATAG
AACCCAACCCCAGTTTCAGGAGAAAAGCTGAACCCACGACCAAACCCCTCTCCCGGTCATCAAGTGGACACATGAAATAGGCATATGGGTAAAT
ATTTCTATGGTTCTAAAAAGGAGTCAAGGCTCGACACATTCATTTAAGAAGCTACCTGGCCAGGCACGGTGGCTCACGCCTGTAATCTCAGCAC
TTTCGCAAGCCAAGGTGGGCAGATCATTTGAGGTCACGACTTTCACACCAGCCTGGCCAACATGGTAAAATCTCGTCTCTACTAAAAATATAAA
AATTACCCACCTCTCCTGGTGGGCACCTCTAATCCCACCTACTCCCCACCCTCACCCACGACAATTACTTGAACCTGGGAGGTGAGGTTGCAGT
GAGCTGAGATCACACCACTGCACTCCAGCCTGGGCAACAGAGTGAGACTCCGTCTCAACATAAAAACAAGGCTACCAAGATGGGGGCATATAGA
GAGAAGACAAGTCTCATCATATAATCTTGGGGATCCATTAAATTGTGCTGAAACAACACACCCACACAGAAATCCTATTTCCACACCTAAGAAA
AAACCCACGAGAATTAATGGTCATTCGATCCAAATCCAAACCACCTCAAAGAATGTGCTCATCCACCTCATTCAGTCTTCCACGAACGTCGACA
AGAATAGGCTACTCTACTTGCAAACTACCAACTCCCTGATTAGAATAGAAGCCAGACCAACACTGTTAAAGCAATCAAAAATAAACCACAAAAG
GCAGCACATGTAGACTCTCCTTTCTGAATCTTTCAAACACAGGTGAGGGCAACAGTGACCTAACCACAACTTCCGAAGCACCTTACGGGTTCTA
AGACTGGGCTCCATACACACACTGCTTAACTTCAAATCATAGCCCCAACACTCAATAGCTGTGTGACTTTGGGCAAGTTAACTTACTTAACTTT
CCTAAGTCTCAGTTTCCTTGTCTAAAACGGTGGGACAAGTAGCCCCTGTTTCATATGGTTATTGCAATGATTATATGAAAATGTATGTGAAGCA
CTCAGCACAGGGCAGAGACGTGGTAAGCAACCAATTAACCTCACACATCAATTTTAACAGGGTCAACGAGGAAAACTGGGAGGAGGAGAAGTTG
CCATGCAGGACCGACAGAACATCCTCCCCTCAACTACGCTTTGAGGAGGTGGAAGGGAGGGGACTGACGACTTGGAGGGCTTGTGGAGAGGGAG
GATGGGGAAGTAGGCCTTTTCAAAACCCAACTTTGGCTAAAGGATCACCTGCTGGTTACTGAATGAAGCAGCTACTTCTGTTCCATTTTCTTGG
GGAGTCATGCTGACGCATCTCTAGGAGCTTCTCTCACTCCTAGAATCTTCCAGTGTTTCCTGGAGCCTGGTCTCTGCCTCTATCCCTCCCACTG
CCTGCTGCTTCTCTAGCTGGAGGCAGCTGGACTCAGGCAGCCGCAGCATCTCCCAGCAGCACACATTGGCAGACGCGCATTCAGAGAGAGCTGC
TTCACAACTTCTCTTTGCGCTATACCCTTCACACAGGCCAAATCCTTCTGTCCCCGTCACAGGTACAGATACTTTCTATCTTCAGGTATTCCAA
ATGATCTTCCATCATTAAGGTTGCTTCTGAAACACAATGGTCTCCCTCCACCTCCCAGCTAATCTGCCTATTTCCAGTGTCAGTTCCACCAAAC
TGTTCCCCCTCTGCTGCCTCTCATTCTTCTCATACCTGTTTAAAACCCTGTCATAATTCACCTTCCACTATCTCCACCAGAACTGGGAGCGCCT
CTCCCACCTGCCCACCCTACATTCCTTATATCTTCACTTTTACCCTCTCCTCCATCCTCACCAAAGCTATGGCTCTGCATATATTGCCTCTCTT
CTTGCAATATCTTTTCTTCTCTTCCACGGGCTAGCAACCCCTCATCTTTCAAGAATAGTCTCCAACTAGAAACCTTTCCTAATATTGCCCCCAC
CACCACTGGTGAGTTGGATGTGCTCCTTTACTCAAGAGTTCCCACAGCAACTGGTGGCTGGCCTCCTCTTCCCTATTATTCCTTATACCTTACT
GTTACAGCTGGAGAATACCTCTGTCTCATTATCACACTACGCACTCTTCCAAAATGAGGATTAGGTCTTATAAATAGCTCTATCTCTAGGGCTT
AGTATAGGACCTACCAACCTCAAATAGGTAAGTAGTACCTATTTGCCTTCATTCCTCAGGGAATACTGGTTGAATGAATAAATGTCCTCATTTA
GCATGTATTCTGGCAGGAAGACAATCCACATGCAAGTGGACTACTTTTCTTGCCCCAAGCCACATCCCCACCATCCTACATCTTTCTTCCTTGA
CTGCCTCTCATTTCATAATTCAACACTTGCCACCTTTCTTATTCATACTCACCTTTTTTTTTTTTTTTTTTGAGATGGAGTTTCCCTCTCGTTC
CCCAGGCTGGAGTGCAATGGCGCAATCTCGGCTCACTGCAACCTCCACCTCCCAGGTTCAAGCAATTCTCCTGCCTCAGCCTCCCTAGTAGCTG
GGATTACAGGCGTGCACCACCACGCCTGGCTAATTTTGTGGTTTTACTACACAGGGCTTTCTCCATGTTGGTCAGGCTGGTCTCGAACTCCCGA
CCTCAGGTGATCCTCCCCCCTTGGCCTCCCAAAGTGCTGGGATTACACCCCTCACCCACTCCCCCCGCCCTGATTCTGACCTTTTTATTCCACA
CATCTCAACTCTTCCTCAGCTGTTTCATTCTACCACCCAAACACCTTTTCTCGTTGTTCCTGAGATCACCAGCCTCAATCTCCTTTCTTGAGCA
CCCAGCAGTTTCTTTTCCAAGGGTATCGGAATCTTGAAACCCCACCACATTCTTAGGCATGGGCGGGGAGAGCTGCTAGAAGACATCACTGTCA
AAGAATTGGCAAAGTCAACAATGTGCCAGCATCCAAGGGTCAAAACCTCACCTGGGACATCCTGTTCCACAGCATGATAGCCCCACAAGGGGTG
CCAAAACCATCACAAAACCCTTGTGACTGCCCACCAATCAAGTACACATTACGACCAACCCATCACACTCCAATTAAGCAAAAGGCGGAATCAA
TTTAAAGGTGCCAGAACAGGAACCATGCAACCAGGCTTCAGGAGAAAGTAGGAACGAGGGTCCTGAACTTCATTCATCAGGGTCCTCTCTCTCT
CTTTTTTAACACAACGTCACACTCTGTCACCCACGCTGCACTCCAGTGGCACGATCTTCCCTCATTCCTCCCTCACCCTCTCAGGTGTTCCTCA
CCTCAGCCTCCCTAGTACCTGACACTGCAGGCACACACCACTATCCCCACCTAATTTTTGTTTCTACACACACCATTTTGCCATGTTGCATACC
CTGACTTCAAACTCCTAGGCTCAAATGATCCAGCCGCCTCACGCTCCCAAAGTGTTAGGCTTACAGCTGTGAGCCATCTGGCGGGAGGGACTTC
TATCTCTTATCTCTTCTCTTCCTCCACCCACTTTCTCCTATATCCTCCCCCACTTCCACGTTTCTCTGGCCCTTCCAACACCAACTATCGCCAG
GAGCACCTCCCTAATCTACATACTACACATCTATCCACAAGAAGACAGATGGAACCACTCTTTTCTTCTGAATTCTAGAACCTTAACCAAGTTA
ACCTGAGTGCCTCAGCTTGGATCAAGTGCCCATCCTCGTCCAAACAACTGTAGCAGCACATCTCTGTATTTGTGTATCTTGTTCCTCCATAATC
TTGCAGTCCGGGGAAAGTGTGCAGATGGATCGCTCCCTGAAAAGCAAGAGTTGTTTAGACAACGCTAGAGGTGTTCAGTTCTGCGAGACAAGAG
TTGGGCCAGGTTGATAACTAGCCTAAGAGAGTCGGAATCAGATGAATAAGAACTAGATCTTCGGTCACAACCTAGATCAAGAGTCTTGAGGTTC
AGTCATTTCAAAAAGCTCAAGTCTCATTCCTTCTTCCCCTTCTGGCTAAGATCATCTCCAGTATCTCTTCTTACCAGTTTAATATCTGATACAT
CCTCTATCTCAGGATAATATAATAAATAGATTTTTGGAGCAAGGAGATCCAATAGGAGCTTGTTCTGTCCACTCTACACATTGGCCTGGATTCC
AGTACCTCCAGGAATGGTGCACCCTCTCTACCACCCCCAAAAAGCCCAACTCTCAAGAGGTAGTCTGGAAAAAAGTGTTTTTGGATTTACCTAC
ATTTTTTTTTCCAAGATGGAGTCTCGCTCTGTCACCCAGGCTGGAGTGCAGTCGCCCCATCTTGGCTCACTGTAACCTCTGCCTCCAGGATTCA
AGCAGTTCTCCTGCCTCAGTCTCCCAAGTAGCTGGGATTACAGGTGTGTGCCAACCATGCTCAGCTAATTTTTCTATTTTTAGTAGAGACAGGG
TTTCACCATGTTGGCCAGGCAGGTCTCAAAGTCCTGACCTCAGGTGATCTGCCTGCCTTGGCCTCCCGAAGTGCTAAGATTACAGGCGTGAGCC
ACTGCGCCCGGCCTAATTTTTGTAATTTTAGTAGAGACGAGTTTTCACCATGTTGACCAGGCTGGTCTCGAACTCCTGACCTCAGGTGATCCAC
CCCCGCCCTCGGCCTCCCAAAGTGCTGGCACTACAGGCATGAGCCACCGCTCCTGGCCGGGTTTGCCTACATTTTGGAGATGCTGCACACCATG
TCCCTCTTTTCAAGAGTCACGTAAGTATTTACATATGGAAGACCCTGAGAACGCACCTGGGAAAAAACCTGTTTAACTGTAAATATAATAATGT
ATGTATTTGTATGTGGAGCTCTTCTTTTAGTCGATTATCTATAAACCGTCCACTGTTCCCAATGCATACTTTGGAGACAGACCTACACTACCAG
TTTTCACCTGCTAGTTATTTTTATAAGGGTTGGGATGGTCCATGAGTGAACAGTCTTGATTTAAAGCAGTGTTTCCCAAACCTCATTTATTTGC
CTATAGCCTTCCTGGAGTTTGTCTTATATGTCCGCTCTGTGCACTATTCCTATATGTTTTTATGTAAATCAATCACTTTTTTAAAAAGCCTCAT
CCCAGTTATATCCTTAAAACAATGGTTATTTTTTCTCATACACATCGAAATACATATCTATTAGAATTAAAATGCTTGTCCATGCACTATCAAT
AATCTCCTCAAGTACCATATGGTGGGGACATGTGTTTGAAGTGTTAGGAGTCAGGAGTGGTCTCTGTAAGACAGAGTAAGTCAGTCAACCTCTG
AGCACTGCCAGCCACACATGCTGGCAGAGAAGTTCAGCCTGTGACAAGACTGCCCGGATATTTCCATGTCTGTGCACGGGGATGTCCTCGGTCT
GCCATTGTGAAGTCTGTGCTGTTTTCATGCCTACCTAGTCTTCTTTGGAGGGGCCTGGAAAGCAAATGCAGGCTGGGTACAGTGGCTCATGCCT
GTAATCCCAGCACTTTGGGAGGCAAAGGTGGGCAGATCACGAGGTCAGGAGTTTGAGACCAGCCTGACCAACATAGTGAAACCTTCTCTCTACT
AAAATTGCAAAAATTAGCTGGGTGTGGTGGTACGTGGCTGTAATCCCAGCTACCCGGGAGGCTGAGGCAGGATAATTGCTTGAACCCAGGAGGC
GGAGGTTGCAGTAAGCCCAGATGGCACCACTGCACTCCAGCCTGGGTGACAGAGACTCCATCTCAAAAAAAAAAAAAAAAAGAAAAAAAAGCAA
ATGCAGATTCTCCCTGGGAAACGGGGGGTTGGGAGAGTGGAGTAGTGTGGATGCATTGTTATTCCTGAAGGCCGTCCTCAAATTATCTTGGACT
CACTGCAGCAAGCAGATGCCTCATCTTCTGGCCAGGGATTGATCCTGAGATGGCAAGAGTATGAGAAGAGACACCAACAGCAAGGCAAAACTGG
CTCTCGCAGCTTGCTTCAGAGAAAGAGTATAGCTTTGGAGTCAGGCCGACCTGAGTATGAATCCAAGACTCAGCACTTACTAGGTGCCTGCCCT
CAAACCATTATTTTTTGATAGTCATTACATACCCACAATGATCACCATGGAGTGGATGACGAAACAGGGACTCAGAGAAGTTAAACAACTTGCC
TGATATCACACAGGTAACAAACAGCACAGCAGAATTCAAACCTAAGTTGATTTGACTTCCAAATGTCTGCTCCTAACCAAAGCAGGGTAACCTG
CTTTGATTTGCCCATTTCTTGAGCACCTCTTCTGTAGGAGGGCCTGTTGCTGGGTGCTGGGAATTCAGTGGTAGGCAAGATGGACAAGATTCCC
CCCTCTCACAGACCTTGCTTGCTATAGGGGAAATAGACAGGAAACCAATTACTACATTTGTAACTTAATAACAAGTGTCATAAGTGTTACAAAG
CAGATAGATACTATAAGGCACCTTTTATTTTTATTTGTTTATTTATTTTTTGAGACTCACTCGCTCTGTTGCCCAGGCTGGGGTACAGTGGTGC
GGTCTCGGCTCACTGCAACCTCTGCCTCCCAGGTTCAAGCGATTCTCCTGCCTCAGCCTCCCGAGTAGCTGGGATTACAGGTGTGAACCACCAC
ACTCTGCTAATTTTTGTATTTTTAGTAGAGACAGGGTTTCACCATGTTGCCCACGCTGTTGTTGAACTCCTGACCTCAAGTGATTGATCCACAT
TGACCTCCCAAAGTGCTGTGATTACAGGCGTTAGCCACCGCGCCTGGCCTATAAGGCACCCTTGGTCAGCCAGTGGAAGCAGCGTCAGTAGTGC
GCCGGAGAGTCACTTTGAAAAAGTGATATTTAGGTTGATCTTGAAGCATTCACAAGATGTGGTAAACACATCTAATGGTAAACATGTGATGGTA
AACATTAGATGTGATAAAGATACTGATGGTCCACTGGATGAACAGTTTTCATATTTACTTCCAAATGATTTCATAGATATTATTTCACAGATAA
CACTTAGGATGAAGGTAAATAAAAAGGAGATCAATTAAAATATGAAATAATAATAGTGAAAGTGGTAATAGATGTGGCAATATCCACAAAGGAG
TAATGGTTAGATTAGCATATTGAGAAGATCACTTTGGCAATTGTTGGAATGCAGTTTACGAGAGGGCCAAGAATGGAAGCAGCAATCCCTGTTA
GAAGTGTAGTGTTTTGGAGTGCGGTGTTTTTTGTTGGTTTGTTTTTTGTTTTTTTTAAACCAGGTCTCACTCTGTTGCCCAGGCTGGAGTGCAG
TGCAGCCTCGACCTCCTCCTCGGGCTCAGGTCATCCTCCCACCTCAGCCTCCCGAGTAGCCGGGACTACAGGCACATGCCACCACACCCCGCCA
GTTTTTGTATTTTTTTGTAGAGTCAGGGTTCTGCCATGTTGCCCAGGCTGGTCTTGAACTCCTGGGTTCAAGTGATCTGCCAGCCTCGGCCTCC
CAAAGTGCTAAGATTACAAGTGTGAGACACCACCCCCAGCCACCACATTGTATTAATCCAAGCAGGAGAACGCAGACTTCAAAGGCTTATGGGC
AGCAGAGAGAAGTAGATCAAGCAAGAGATGCAATCAGCGGGACTAGTGGGAAATGAGGGAAAGGAAAGGTGTCAGGATGGGGCTTTTTCTAGCT
GAGTGGAGTTCAGTTCATTCCCAGAGAAGCTTGAGAGGTATCCAACTATAGATGCAAGGTAGCTGGCTGTTGGTCCCTTGTCTTTAGGACAGTT
TGACTTTTGTAATACAGGCGACCTCACAGAGCTATTGTGAGAATTCAGAGCTGGGCACAGAATAGATGCTCTATAAACGGTAGTTCTATTCCCC
ATTTGCCCCGTCCTCCCAGCATGCCAGGGATACAGGGGCCGATAGAGACAGGTTCAGTTCCCTTTGGTCTCTTGCATTCCCCCTTCCCTCAGCA
CAATTCCTGGGCTCATAGCGCATGCTCAATAAGTATTTGTAGAATGAACCAAAGAAATCAGACCCTTGGTGACCCAGGGCTACATGTACTCGGC
GGAAAACTGGGGTTAAACCGGACCCTTGGTTGGTTGAAAAATTAAAATAATTTTTAAAAAGTGGGCGTGAAATAATCCAGAAGTTGGAGGGCCT
GGACAAAGGGCTGGGGAGGGGACTCCTCCCACCAGGCGGCCTCAGGGTCGGCTTAGCAAGAAGGAGGCGCTGGGGAGACCTCTGCGGTCCCAGC
TGCCAAGGTACTCGCCGCTCCAAGGAGATGGGGACGCCGGATACCAGCTCTGCCCAAGATTGCTGGGTCCCGCCGCGGCCTTTAAAGGCCCACC
ACGTGGCTGCGCGGCCAGCCCTGGTCCGGAGGCGGTGCCCGGGTGGCGCCTGCGCACTGCCTAGCAGAGGGGCTGGGGTTCCGAGAAAAAGCGT
CTGGAGAGGAAGGGGAGGCTGCTTGGGGTGGGACGCCGTGGGAGGAGTCGGCTCCTTATGCAAATCACAGCGGAGCGCGCACGGTCCGGAGGCG
GGGCTTGCGATGCAAAGACAGGTCCGTCTGGCGAACAGCGAGGGGGCGGGCCGCAACCCTCTGCCTCTTTCCGCGAGCGCTGACGTCGCCGACG
TGTTGTTTAAAAGCGGCCGCGCAGGCGCAGTGAGCCCAAATGCGAACTTAGGCTGTTACACAACTGCTGGGGTCTGTTCTCGCCGCCCGCCCGG
CAGTCAGGCAGCGTCGCCGCCGTGGTAGCAGCCTCAGCCGTTTCTGGAGTCTCGGGCCCACAGTCACCGCCGCTTACCTGCGCCTCCTCCAGCC
TCCGGAGTCCCCGTCCGCCCGCACAGGCCGGTTCGCCGTCTGCGTCTCCCCCACGCCGCCTCGCCTGCCGCCGCGCTCGTCCCTCCGGGCCGAC
ATGAGTGGGCACCACCTCCACAACGATTCCCAGGTACGGCCCGGCCTGACCCTGGCGGCCCCGGACCCCGGCCTGGCCGTCCCGCGACCCCCGG
CGCAGGCCCCGACCCCAGCCCCGGCCCGGCAGCTTTGACAGGCCGGAGCCCCCGGTGAGCGGCCGCCTGCCGGAGTAGATCGGCTCGCTAGGCC
GCGAGCGAGGGCCGCGAAGTTACAGTTCGAGGCAGGGATGGCTGCCCTCTGTGGCCACCCCCGGGTCCCCGTCCTCCCCGGGGGCGCAGGGTGA
GCCAGACCCCGGCCGCGCGCGCTCGCCGCCGGAGGGGTTAAAGTGGCTGTTGTTTGATATTCTCTCCTTTTCTTTTTCCAGATCGAAGCGGATT
TCCGATTGAATGGTGAGTGTGCCCCCTGCGCCGACTCCGGGGCCCCCCAGCCGCCGGCCGCCTCCCCCGCGCCCTGCCGGTGCCGGGCAGAGGA
CAGACATGGCGTCCCAGAGACTAAGTCCCGGCTCCTCGCTCACCGGCCCCATTGTTCCCATCGCGCCGCCTCTTGACCCCCTTTCCGGGGACCC
CAGCTCCTCCAGATCCCGGCCCTCCCAAGAGGGGACAACGGAGACCCCGTGTCGTCCGCCACCGGGCCTCGGGCGGTCTTTCCGGGCCGGGATT
CCTCCCGGGAAACTCGCCTTGTCGACGGTCGGGACTTACTCTCTGCGCCATTTTCTTTTTCTCTCTCCTCTCCTTTCTGTGCCTGTGTCTCTTT
CTCTCCCTCCCTCGGCTCTTTCCTTGAGCTGCCCAGAATGAGCTTTCTGCAGAGCAAAGCCGTATAAATCACATACTTCACCAGAGAGGGAAGG
CTGGGGGACTGAATTATTATTTTTTAAACTTTATTTTGGGGGTTATTCCCTTTATGCTTCATGTTTGTTTCCTTTTCTGCTTCCAGAATGAAAG
ATTTCCTTAAGCAACTGTTAATTTTTTTTTTGATAGGCTGTTTATGATACGAATTATGGCTCTAAAACTAAAGCCACGCTTTGTGGAGTTCTTT
ATATTTCCAGGTTGTTTTTCCCAAGTTACGTTCTTTGCAAAGGTAGCAGTACCTCCCTAAAGAAAGTTTGCAAGTTCTCTGTGGGTCACCCAAT
GTAAATGTTGGATTTTTAAAAAATTATGTTAGTCACTGGAACTTTTCATTGTGGTCGAGGTTCCCAATGCAGTGTTTTTCTGGAAACGCAGTTT
AGTGTACTTGTTGGAACCAAACATTGTTGAGCATCTCTTGTGGACCTGGTGTGTTGGCCTTTTCGTTTGGGTTGTAAGGAGGAAAAAAAGATTT
TATTTATTGAAGCAAAGAGTGTCCAGTACCCACCCCTGCTTCCTGTTCATTCTTTTTCACGAGCCACTGTTTTTCAGGAATCAAGAACTGGCTT
TTTTTTTTTTTCCCAATTCTTTATGTTTTTGAACCCTTTTCAGCCAAATCAAGGCATTTATTCTTACTGTATTTCCACTTCAGTTACCCAGGGA
AGAGTCCTCCAATAAAAAGCTCATCCTTGCAGGCTTTCGAACAACCCTGTTGAAGGAGAAGTTACCTTCTGTAGTGTGCTGGGAATTCCCACTG
ATTTTGATGGTACATAATCCTGCTCAATTGAGTTTGGGTTCCGGGAACCTCTCCAATGGGAGAAGTTGATGAGTAGGTTTTCTAATGTTGTAAT
CAGCAGTCCATCATGTTAGCCTGCTCTACTCAGAGCAACCTTATGTTCAGAGTTTGAAGTGGGGCATCCAGCAGGAGATGGTACTCAGCCAGGA
GAGAATCTGGTTTAAAGTATGGCATTTCTTGAGCACCAGATATGTGCCAACCACTGTGCTTGGTGCTAGAGCTGATCCTGTAACCATAACACTC
CCTTGCTCTCAATTCAGTCTATTGGGGGAGACATAATTAGTTAATTATAAGGTAACTACTGGGGCATATGTGTACCCAAAATGCTTTGAAGTAT
AGGGGAGTTAATTTTGTCTGAGGGTGGGGAAATTTCTCAGAGGAGGTGACTTCTGAGCTGGGGGTTGTGGAAGACTGGGCGCAGAGAATTGTCA
GGGGTTGGAGTGTTTCTTAAGTACGTTTAACCAACCTATCACGATTTTAAAAGAGGGAGGAGGTGAGGCCTGCAGTTTAGAAAGCCACAGTATA
GACATGTTTCTTTGGGATACAGGAGCAAAGAAAGGATGTGCAGGTGAGGATGTAAGAAGTCCTTTTGACCTTGCTAGAGGAAGGTGGGATCTTT
AATATGGAATGTGTTCTTTTGGCTGTCAGGCTGGGCTGAGTAGGTAATGTAAGTAATGAAACAAAGTTTAGCCCTTTCTTGTGAGAAAGTTAAC
TTACATGGTTGTCACCTAAGTCACGTGAATGATTGTGAGCTGGCTAGGTTATTGATCTTTACTGACATTTTTATAGAGGCTGAAGTAATCTGCG
TATGTTGACTTTTAGAATGTGTTGGCTCACATTTAATAGCAATAATAGCTAATATTTATTGAATACTTACTGCCAGGCATGAATCTCAGCTCTT
TCCATGGATCATCTAATTAAATGTAATCCTTGGAAGGACCCTTGTGAGTTAGGTCCTGTTATTTCTGTTTTGTAGATGAGGAAACTGAGGCTTC
AGTGTATTTACTAGCTTGCCCAAGGGCTGTGAGTTGGCCACTCTAAATCCCATGAGCTGAGCCACTATGCTAGAGTGATACTATATGATACTAG
CATGAAAAAATAAAAAGCCACAAATCTCTATTTTCTTACAGAGCTGAAAAAGGAGAGAAGGAAGGATTTGATAATTTGTCATTTGCTTTTTATC
TAAACATGCTTTTTGAAGAGAATCTGCCACCATAAAGTTCTGACATTGGTGTAATTATTCTTTGTGAATATTGTCTGGAAGATGTGGTGTTGGT
AACTATGCTTGCTATCTCAAGTAATACATTGTTAACCAGTGATTGATTTATGTAACTATGAAAAAAACTTTAAAAAAAGTAGAAATCAATGTTT
AATTATGCCCAGACATGTCATTATTGAACTTTGTATTAAGAGATTGTTTAAGCGTGATTTTAAGAGGGCCTAACCCATCTTACAGATTAACTGT
ACAAGTTCATATATTAAAATCAAACGTCTCTCATTTTGCCTGCCCTCATCCCCTAAGATATAAAAGATTCCCCCCCGTCCCCCCACTTTGGAGC
TCATTCTTCGCAATATTTAAAATTGTAGCTCTTTGCATGTAATTTAGAACATTCTGACCCATACTCAACTGCTGGGAGACTCTGTTATTTATAT
TTAAAATGTTGACAGTGAGTTAGGAATTTGGAGCAGAAACCATATCTAAGTCTAAGTGAAAAGACTTCCTAAGTATTGGTAGAACTAACTGTAG
TGGGGCTGAGTCATTATTACAGTACTGTACAGGCTGCGTAGCTATCTGTAGAATTGATAATGGTAATACCAGGTTAAAGTGTTAGCGATTCTTC
TGCTGTTTCAGAGGTCTTGAAGTTTTTGCCTGTCTGCTATGGAGATAGGGAGTTCTCCCCCATTTGACCCCCTTAGCAAAGCACAATACTTGGC
TCCATCAGTCTTTAGTCTAAGAAGTGATATATAGGATCATGGTTTGACATCATCTTGTGCTGATTCTGGTTGGCTTAATGGAATCAAATTGGAT
GAAGGTAATTAAATATGAATTGTTTCTGATGCTAATAGATAGTTGTACTTCTGTCTGTTGGGTATGGGTTGGCATGGGCGGTAGCATACCTGAT
TGCGGTGGCGGAATTTCAGATAGCTGATGATGTGGCTATCTGAAAATTGCCACTACATTTTTAAAACTGAAAAGTGATGGTACCGCCAAATGAA
ATCTGCAGCATCCTCAGACTCAATCAGCAAATACTTAAGCATTATGCTCCAGGGTGGTTTTCTTCCCTTACATTGCAACTGGACTCCTTGAGGC
ACCTTTTTAGAATTAGTGAGCCGCTAGGATTTTTATACTATGCTTTGACATAAAAGAAAGTTGCTTGTCAATATGTGTTTTTTTAGTTTACCAG
CATATCAGATCTCACAAACAAATTTTAGGCTCGGATTGGATGTTTCTAGTAAAAACAAGAAAATCCAGGCATCACTAAAAGAATCAGAAATACG
TTGTGGCTTGAAAGGTGCAAACTAAATAATATAGCTAGCAGTAACGGTCACCAACTCCTGCTTTTCTTTGGTTCAGTAGATAGAGTAAAATGGA
AAATAGGAAACCAAGGTCAGTCCCCAAATCTCATTTTAACCAATAGCAGCAAAAAAAAAAAAAAAAAAAGGAATTGAGTCTGACTTTTTCCCCT
TGCTGTCAGCATAAAGATGGGGAATTGAGAGAGTTGGAAAGACATTCATTAGTTTGTTCAGATTATTAAATAACATGTCTTAAAGTTTATATTC
CAAAATTGGGCTGTGAGGTTTTTTTGTTTTGCTTGTTTTCAGTGCATGTGTGGGGTGGGGAGGTGCAAAAAAATGTTTGCCTTCTAATATACAG
CTTTGTTGTATATTAATTATATAGTAACAGTTGTCCCTGCTGCTAATGGTATGGAAAATGATTCTGTTGTGCTTTCTGAATACATATCATTTTA
GAAGTTTCAGATTATACTAAAACCATCTTATTTCATATTAGTTGGACATGAATGCATATTTATATGACACATCATTATCAACTTTTGCCTTTTG
CCATCTTTTTAGTGCAAAGTAAAACTGATATTCTTCAAATCCAAAGTCGTTTCCTCAGAAGTGTGTTTTGTGAGGGATTGCATCTTTCCCATGT
GAAACTGTGTCGAACTCCAGTAAACTGTGTTTGCTTGGGAAAATGAATTCAAATGTAATTAGTTTGAATGAAGCTGTGGAAATGTTTGAAATGA
GTATTTTGACATTGGACCTGGAGGTTTAAAAAAAAAAACCCTATTGAAAGAATAGTAAAATATATTGGATGTGGAGGTGAGAGTCAGTTGAGTG
AATTTTCTGTGTTGTATGGTTTAGATTATCTGACTATCCCTTAATTATTTTGAAACCATTAACTTTGGATAATAAGGGCATTTAGGCAAACAGT
TGACACATGGATTTCTTAAGGTAAAAAGTTTAGAGACTTCCAAACTAGTCCTTAAGAGGCGAATGGGTATTGTGTTTAAAGGAAGCTTTCAGAT
TGTGTTTAAACTTCGAGTAGTAATATTTGGAACTGTCTTTTCTCCCTATTTCCATGCCTCATACATCTCAAGTTTGAGCAGCAGCTTTCCATCC
CCATGGCTCCAGGCCAATAGCTGCAGTAGAAAATCTAAAATAGAGGCCAGGGTTGGAATAATTAGCATTTTGCCAACCCCCACCCTCCCTGCCA
CCTGCCTATCTAGATGTGGACAGTATGTGCTGTGTTGATAGCATAGGGCCTGGAAGGGTGGAGAGGGATGTAAATGATGGGCAGGGTGGACAAA
GTTTGATTGGGGGTTGCGGGGAAGCTTGTAGAGGAATGTCTGTTAGATATTGATGGTATTTTGGTGGCGTTATCAGAAATGGCTGATAGTTTGC
TGAAATGATTTAATAGTAAATAGATATTGTTGAACATTTTCTGTGTGTATAGGAAAGATCCTACATATTTTTCACACTGGGAGAGAAGTGATGT
TACTGGTATCTCATGCATATCTCTGGAGTATATAGATCGTAATATCAACTTTGCCTATCTGTATCGAAGTTTGTATTAACTGTCTTTATGTGTG
TCATTTATGTCTCTTAACGTTTAGTAAGAAAGAAATTCCAAGGAAGGTAGGCAACTTTTAATTTTAACTTCTTTCCTTTACTTTTTTTTTGGGC
GGCGTGGGGGGCAGACAGGGTTTCTGTTTGTTGCCCGCGCTGGAGCGCACTACTGTGATTGCAGCTCACTGCAACCTCTGCCTCCTGGGCTCAA
GCGATCCTCCTACCTCACCCTCCCAAGTAGCTGGAACTACAGGTGCATGTCACCACGCCGGGCTAATTTTTGCATTTTTTGTAGAGACGAGGTT
TCTCGCCATGTTGCCCAGGCTGGTCTTGAACCCCTGGGCTCAAGCAGCCTGCCCACCTCAGCCTCTCAAAGTGCTGGGCATGAGCCACTGTGCC
TGACCCTTAATTTCTTTTTGGTAGGTATTCATTTTTCATTTGTTGTCTTAACCTTCTAGTTTCACTCACTTACCTGTCTGCTAAATTTTTCTGA
AATGTGTAATAAATGTTTTAATTTACCATCTCCCTTCTACCCTTCCCATCAGATTTGGGGTGGGGAGGCATCTCTACCAGGCAATTAATTCCCC
TCTGAGCCTTTGAAATTAACATTTTTCTTTCTGTGGATTCAAGGAGCCTAAAGAACACAGTTCATTAGCATTCCTAAGAAACACCTAGGTAAGC
TCCAACAAAAGATGTTAAAGCAATCAGTATTTTTAGGGACTTCAGATCTCCTGTATTTGTTGTTGTAAATGATGATGTTGGGAAAAGAATTTAA
CAGATCCCTCGGTATTCTGACGTTTTCCTATATTTTAAGAACGAAACCGACCCAGCCAGCTTGTGGTCCCATTATTCTTTATATTGTTTTTCTT
AGCACACGCTAGCAAAGATACATGCCTAGCACTTTTCTTGGCTATTGGAGTTTATACAAAAAAAGTTCTTTGTTGAAGGTTAGTCCTCCACCAG
AATAATACACCCTTTTTCCCCTTAGTAGAAGTCCAAATCTTAATCTTGCTATCATATATCTATGAATGCCCAGATTTATAAAGCTTTGTAGGTA
TATTACTGATTGTGCAGGAGACTAATCTAGGAGGCTTCAGGATCATTGTCATTTGTGGATCTCATTGGCTAAGACCTTTTACTTAACCTGACTC
CAGTAAGGTAGCCCCTTTGTTTCCTTGTAGTTCAGTATGTGAGCTCTTGCCAAGCAGCAGAAGTGGGATGAAGGGAACTAATTTTGAAAACTTA
TTAGGCACCACATTTTTATGTGCATTAAATCTGAATTGAATAAAGATAATATAGATTGCATAGGCTTATGGGGAGAACCAGAGACCAGGAAAGG
AAACGTTTTTTTCTTTTTACCCTTTATGCCTTTTCATGTCCCCTTTTTGATAACATGCTGCCTTGGTCCAGATTCTGGAAACCCCACTCACGCT
GTAATTGGTAAGCTTCTAGTACAGATGTCAGAACCTGCCCTTGATTATCTGCTGTTAGAGATAGCTTGGACCCTTTGTATGTGGAATGTTAGGT
CTAGGGGAACAGTGGGGGTGTGAGGAGACAGCTGTGGTAAAATAGAAAATTGGTGTAGGATTCCGAAAAATCGAGATAAGTCCCTGTTCTTCCT
ATGAAACATGATCTGACCACTGAACCTTGTTTTCTTATCCATAAGCAAGGATAATATTTATGTTTCACAAACTGTTGAGAATTAGATAAGTATA
TGAAGGTGCCTCTCTGTCCTCATTGTAAAATGTAAACTTTGATGAGACAGCCTCTATTCTCAACTGGATCTTTATTTTGAATGTTAGGTTTGAA
ATAGTGATTGATGTTGGGGAGTCTGTCTTTCATTGGTATTCCCTGTTAAAGTTTTAAATCTTCTTGACATTTACACAGTGAATTGTAACATGTA
ACTAGTGTTGTTGAGTCTTTACCTAATTCAATCAGCAGAGCCTACACTGATGTCTTCCAGTTACAAAGCGGTCACTGTAAAGTAAAAGGTGTCT
TCATATCTAGGTTTGCGCTGAGAAGGATCGAATTGTATATCTAGTTCCTACTTTTCTTTTTTTTTTTTTTTTTTGAGATGGAGTCTCACTCTGT
TACCCAGGCTGGAGTGCACTGGCGCAATCTCGGCTCACTGCAAGCTCCGCCTCCCAGGTTCACGCCATTCTCCTGCCTTAGCCTCCTAAGTTGC
TGGGACTACAGGCGCCCGCCACCACGCCTGGCTAATTTTTTGTATTTTTAGTAGATACAGGGTTTGCACCGTGTTAGCAGGATGGTCTCGATCT
CCTGACCTCGTGATCCACCAGCCTCGGCCTCCCAAAGTGCTGGGATTACATGCATGAGCCACCGCGCCTAGCCTAATTCCTACTTTTCTAAGAA
TGTTTACACCAAAGTGGTCATTGCCAAAGGAACTTGTAGCAGTACAGAAGATGGGCTCAGAAATGAAAGAAGTCTATTGATGTGACTAATTTCT
CAACCATTTAATAGAATTATGGATTTTCTATTAAAAATAACTTGACCCTTTTCGATGCATTTAATAAAATGCCAAGTTCATTATTTGTTAATTT
TTTAAAACCACAAAATTCAATATGTCTTGTGTCAGCATATTTTTTCAAAGGCCACTATTTTACTTCTCCCCCAAAACGTTTTTAAATTAAACAG
AGAACTCTCAAAGCACCTGAGAATTACTTCAGTTTTTCATTTATTCAAATAGTTGCTTAAAGTGACCAACTTGGTTTTCTTTGAAATAGTAATT
CTTGGGAATAAGAATACCTTAAATAATATAATAAAGCTTATGTGTGAGAATTTAGTACCAAATGCCGTGTTAACAGTTATGCGTGAGTATAGGC
ATTTGGGAGATTCCATGAAAAAATTTGATAAAGTCAGTAGAAATTTCAGTAATGGCTCATAAGTCTAGCTAATGCTGAATATTGAATTGCCTTG
AATTTGTTTTTTTGGAAGTGTTACTTAAACGAAATAAACAAAAGTCATTTAAGGATGTGTTCACTAGTGTTCATTAGGTTTAAGCCAAATGGAT
CTTAGTAGAGATCTTACGCCATTCAGCAAAAGGATTATCACAGAAGCATCACAACAGGCGCAGACTTAAATTCAATTAAAAGTAGGCAGGAGGA
CATAATTTTGTTGTATAGAAATTATGGAATAGGGCCTAATTTTAAAGAGTCTTCAAAGATAAACATATCAGGAAAAATATAGCTCATAAAATAT
GGGGAAAAAAATCTGGGAAGATGAGTATTTGTTAATGCTCTGTAGCTCCATTGCCCTCACTATACAAGGAATGTGCCCACAATAGGGTTGGATT
TAAAAGTCTTCAAAGCTTTGGTCTCTTTTTCTTTCGTTTTCCTCTGATACAGAATGCGAATAGTCTCCCGGCCTCCAACCCCCTCTACATCCCC
ACCACCCCATAATAGAGACTTAAAGGGAATTTAAATTCGAGTTCTTGAGCTTCTGCTCAATGGCTAGGTCTTTGAACAAGTTAATATCAGAGCC
TTAGTTCTTCACCTATAAAGTAGGTTTAATACTTACCTTACAACATTGAGGATTAAAGAGCATGAGAGGATTACTTTATGAAAGGTGAAGCTTA
GAACAGAATTTTTAGTTCCTTGCCAATGCTAAAAAACAACAACAACAACACCTTTTTCAGAAATTGCTTTATTAGGCTTTTGTACAAAGGGAAC
ATCTTTACTTTCTTTAAATTTGCACATATATCTATTGTGAAATTTTAACCAAGCTGGAGGGGTCACTAGGGAGCATTGAGTATGATACTATTGC
TGTAAATCATAGAAAGGCTGGCTCAGGAGGGTACTGACTTGCTTTGACTTTGCGGCAACTTCAGTTGTCCAGTGACAAGGCTTGCTGCATACAC
ACCACTTTGTTTGTTTCTCATACCTTAATTGTGGATTAAAAATCTTTGGGGCAGGCCTGGAGGCTCATGCCTATAAATCCCAGCACTTTGGGAG
GCCGAGATGGGCAGATCACTTGAGCCCAGGAGTTCAAGATCAGCCTGGGCAACATGGGAAAAACCTGTCTCAACAAAAAAAATACAAAAATTAG
CCAGGTGTGTTGGTGCATGCCTGTAGTCCCAGCTACTTAGAAGCCTGAGGTGGAAGGATTGCTTAAGCCCCGGGAGGTGCAGGTTGCAGTGAGC
CGTGAGCATGCCACTGTACCTACCCCGTTTTGGGCAACAGAGTGAGACCCTGTCTCAAAAAAAAAAAAAAATCATCATCATCTTTGGCAGTGAT
TAACTTTCTCCTTTCACACTTGGGATGGTGCAATATTTGGCATCTACTTAGGAAGCTAAAATTTTCACGTTTAATTGTAAATGGAAAAGTTTAA
GTTGCCATATTCATTCATACAAACATTCAGAAAGTGAATATTGTGACTAGACACAGATGAACAAGACAAACCCACATGCCTATCAGGATGAACT
TTGAAATTAGTACGTTGTAGGTATATACAGTCACTTCCTCTGTTGAGATTTTTCATTAGGCCTCAGTACAAAGGCTGCTGTCATACAACATGAA
TCCGGACTGGACGTACTTTCTGGCAGTTAATATTTCACTATCAGTCCAAGAGAGTGACGTCTCCATTTAGTCCTAATCGTGAGTTTTTCCCCCC
TTAAAGAAATTGGAGACCTAGGACCTTGGATTTCCAAAGATTTCCAAACTCTTATTTCTTAAAATGTTACAAAAAATGGTTTTTTTTTGTATAC
CTATATACCCATTAAACTTTTTTGCCTGAAATAGGCAAAAAGATTTAAGTTCTGTGTATTTATTTCCTGTTATCTTTTTGTATCAGACATCCAT
GTTGCATGAATTTTAGAAAAATAGTTTTCAGCTGGGCTTGGTAATGGCTCATGCCTGTAATTCCACCATTTTAGGAGGCCGAGGCGGGCGGATC
ACGAGGTCAGGAGATCGAGACCATCCTGGCTAACACGGCGAAACCCCGTCTCTACTAAAAATACAAAAAACTAGCCGGGCGTGGTGGCGGGCGC
CTGTAGTCCAGCTACTTGGGAGGCTGAGGCAGGAGAATGGCATGAACCCAGCAGGCGGAGCTTGCAGTGAGCCGAGATTGCGCCACTGCACTCC
AGCCTGGGCGTCAGAGCGAGACTCCATCTCAAAAAAAAAAGAAAAATAGTTTTCACATTTCCTGAGCAGGTAGGTTGTCGGAATGGCTAGAGAT
GGTCACATTCTGTGTGAGAAATCATGTGACAATAGCTAATTCAAGTATTTGAAATAATTTAAATCCTTGAACTGAATTGATTGAAATAAAGTAT
TTAAAATCACCATCTTCACATATTTGAAGAACTAAAATCTTAGACAATAACTCTACTTAAAGTCCTCGGAAACTAATTTTATGAAAACTATTCA
AATATTATTGCCCCTTCCTAATCCATGAAGTATCTTGATATCGGTAATACATATAGCTGTTAAAAGCTCTTCAGAGAAACATCCAGAGTACATA
GTGGTATTACTTACAGTGGTTTGAATACTAGAAAAGTGATCGGGACAAAATGTTAGACTTACTGTGCCTTAATCATACAGTCAGTGCATTTTTG
CAGGAATGGATGCAATGTAAGTACTTCTTACTATTTAATACCAGAAATGAAGAAACAGTAGCTTCTTAGGAATTGATTGTGGCTCAGGATCTAT
TTCTTAATTTTTAATGCTTCCACTTAACCAGGAGAGGAAAGGGAGGAGAACGAACACAAATTTGTTAGTCTTTATAACTACTGGCTTGTTACAC
TTGATTTCCAAACCACAAGTGCTCCAGGGCCAGGCACCCCACACTGAGAAGTCCCTAAGAAGTGCATTTATTGGAAATTTGATTTTTAAAAAAA
ACCTCATCTCTACTAAAAATACAAAAGATTACCTCGGCCTGGTGGCGCATGCCTGTAGTCCTAGATACTCAGGAGGCTGAAGCAGGAGAATCAC
TTGAACCTCGGAGGCAGAGGTTGCAATGAGCCGAGATGCCACCACTGCACTTCAGCCTGGGAGACAGCGAGGCATCGTCTCAAAAAAAAAAAAA
AAAAAAAAGACCATGTTAGGTTCTGTTTAAGTAGTATTTCTTATACCCTCGAATGAATGAATGCCTCTTAGTCTTTAGAATTTCATAAGGTTTA
GCTAGGTATACTCAATACTTCTCCATTTTACTACATATTTTTAGGAAGGCACTAGGTATTGTGAACTCCTTGAGAGTTCTAAACATCTCAGAGC
CTGTTTGAGTATTGATGGCATAGTCACAGATACTCCATTTCCTTTCTAGGGAAGTCTGCCTGCATTACCACGTAACCCCTAGCCAGAAGTGTAA
CCACTTTTCTCTGACTTGCATTGAGCTTTTGTTATCCTCTTTTAATATTTCATTCTGTAGTAGTCTGTATTTATGTGTTTTCCTTATTAAGAAG
TGGCCAGCACTTTGGGAGGCCAAAGTGGGTGAATTGCTTTAGCCCAGGAGTTCTAAAGCAGCCTGGGCAACATGTCGGGACCCCGTCTCTACAA
AAAAATAACAAAAATTAGCCAAGCATGGTGGTGTGCACCTACAGTCCCAGCTACTTGGGAGGCTGAGGAAGGAGGATCAGTTGAGCCTAGGAGG
TTGAGGCTGCAGGGAGGATGTTTGCCCTACTGTGCCCCAGAACGAGACCCTGTCTCAAAAAAAAAAAAAAAAAAAAAAATTAAGAGCACTGTTT
CATCCTGTATGGTCAATACAGATGCTTTTACCAAATGTTTCAGTTGTGAAGTGCAAACTCCTTTCTCCCCCCAGCTGGTTGAAGATCAAGTGGG
AGACTTTTGAGAGCTTAACAGTAAGTTTTCCTTGTTTTTCTCAAGCTGATATGCCTAAAAGTCACCTGGGGATGATTGCTGAAAATACATATTC
CTACTCGTACACCTTGAAGTTGATTCTATAGTGCAACGTACTGAAAATCTGCATTTTTAATGTATCTTCAACAATCCCAAATGATTCTGACTAA
TGCAGGTGACCCATAACCTCCTTTGGAGAAGTTGATCTATCTGAACTTTACTATCTTATTTGGCTAAAGATCTATATTTTGCTTCATTCAATTA
CGATACTCCACAGGTTAAGGTATAAAGCTTACAGGGTGATGATGATGATGATGATGATGATTATTATTATTATTTGGAGACAGACTCTCCCTTT
GTCGCCCAGGCTGGGGTGCAGTGACACGATCTGGGCTTGCTGCAACCCCCACCTCCTGGGTTCAAGCGATTCTCCTGCCTCATCCTCCTGAGTA
CCTCGGATTACAGGCACACCACCACCACACCTCCCTAATTTTTGTATTTTTAGTGGAGACGGAGTTTTGCCATCTTGGCCACGATGGTCCCAAA
CTTCTGGCCTCAAGTGATCCGGCTGCCTCAGCCTCCCAAAGTGCTTAGATTACAGTAGTGAGCCACCGTGCCCAGCCAACATTACAGGATATTT
TTAAAGGACCAGAACAGTATTTTTTAATATATGTTTATAAAAGCTTGATACTTCACTCTAAGTTATAGTTTCTACCTTTCAGTGAGCATTTACC
AAATAGCAGATATAGTTAACTGCCTTTGCAGCTGTTTTACTGTGTGTCAGAATCCTAATCCTTTCTCCTACTTCAGTCTTTAACAAGCCATTTA
ATTCTGATGTTACTTTTTCGGATTGGGTGACAAGCAGAGCACAATGAGCACGGTTTGTTTTCATACTAATACTAGGATGGTTAAATCAGGACTT
TAGAAAGTAAGTGGAATAATTTAGTGGAATACTGAAAATCCAGTCTCTGGAGCTCACTGCCTTTACATTAAATTAGTTGGGGACTTCAGCTCCT
GATGGTACTGATTGAAATGGAAGCATTTGAATAAAGCAGTATAGTTGACGCTTGAACAACACGAGGGTTAGGGATGCTGACTCCTATGCAGTCA
AAAATTCGAGTTATAACTTTTGACTCCTCGAAAACTTAACTAATAGCCTACTGTTGACCAGAAGCCTTACTGATAACAGAAACAGTCAATTAAC
ACACATTTTGTATGTTATATGTATTATATACTATGATCTCACAATAAAGTAAGCTAGAGAAAAGGAAATATTAAGAAAATTGTAAGGAAGAGAA
AATATATTTACCATTTATTAAGTGAAAATGGATCATCATAAAAGCCTTCATCCTCATTGTCTTGTTGAGTAGGCTGAGGGGGAAGAGGAGAGTT
TGGTCTTGCTGTCTCAGGAGTAGCAGAGGAGAAAGAAAACCCTCATGTAAGTGGACTCAAGCATTTCAAACACATATTGTTCAGGTGTCATCTG
TGGTTCTCAATCTGTCGTTTCCTGATCACCAGAACTGCATTGTTTGAAAACTTGTTGGACATGCAGATTCTCGGGCCTCACCCATACCTACTGA
CTCTTAAACTCTGGGAGTAGGGTCTTAGCCATTTGGGTTTTAATGAGTGTCTACCAGGTGATCAGATGTGCACTGAAGTTTCAGAACCAATAGA
ATAGAGCAAGATACCAGTTTTGTAGTAATTCAACCTCTTAAGCAGTATTCCTTGTTCTTTCACAAGGAACCATGTATGGAGTTCATTACTTGTG
CTTTCTTACAGTTGTGAAGTTGGAGTAATTCATAATCGGGGGAGGATCTTCTGGAGTATAAAAAAATTTAAGGAGCAGTTATATGATTAACACC
AGAAAGGTTTCTTCTATTTCTACTGAATATATGCCACTTCAGTAGGAGAGGTGTGTTCAGTCTTATTCTTTCTACAGCTAGTGGCCCAGAGAAT
GGCCTGCGCATTTCAAGCCCTGTCAATGAGAATTGTCTAGACAAAGAAGGTCCCTTGGTTGAATTAGTAATAGGATACATGAAACAATGTGTCA
GCACACTTAAAGTCTTTATGGCCAGACAAAGTATGGTCTTAGTGTGTTTGGAGGCCCCTGACCATTGGAAACAAAGGCAGTTTAGGCTGAGCTG
TGGATCCGCACGTAGGCCAGAGGTTTGTAGTCACTCTCACTCTTCCCAGGCATTAAGCGTTCCTTAATGGAAGCAAAGGAGGACAAGCCTACAG
GAATGGGCCTGCCCTAATTGAGCTGGCCTGAGAAGATACATTTTCAGGTTAGACAAGTCCTTTTTCCCTTTTAAGTTCTGCCTCTTGGGAAAGA
AGATGCATTTGTAGTGCAGGGATGTCCTTGTGAAAATGTGACTTGTTATAGAGCAGTATTTGAAATGCAAGTGCCCAGCCGGGCGTGGTGGCTC
ACGCCTGTAATCCCAGCACTTTGGGAGGCCGAGGCAGGCGGATCACCTGAGGTCCGGAGTTTGAGACCAGCCTGACCAACATGGAGAAACCCCA
TCTCTACTAAAAATACAAAATTAGCCAGGCGTGGTGGCAGACACCTGTAATCCCAGCTACTCGGGAGGCTGAGGCAGGAGAATCGCTTGAACCC
TGGAGGCGGAGGTTGCAGTGAGCCAAGATCATGCCATTGCACTCCAGCAGGGCAACAAGAGCAAAACTCCTTCTCAAAAAAAAGAAAAAAAGAA
ATGCAATTGCCCATCTTAAGGGTCTTGTACTAGTTAATTACTTTCAGAGATAAGTAAAAAAATTCCAGTGTTGTCTTCTATTAAGAGAGCAAAG
GTAGTTAGTAAAAATGGGATTTGTTTGCCCTGCTCCCGAAGCTTAAAATAATTGCTCTATCTCAGTTTTGTCTTATGCTTCGGCAGACTAATAA
CTTCAGTTGAAATGTTTTCTGGGCAGGTGTTCATTGAGGGCCTGATTCTCCAGAGGTTCTGTGCAGAAGCAGGCTGCTTTGAGATAGCCAGCTT
AGCCAGGCCTGGGAACCACACAGAATAGTGCAGTACCATGTAGGGAAAAGTCCTTGACTGGGCTGCAATAGGCAGAGTGGAGAGTGCTAGGAAG
CAGAGTATTTTCCTGGATAATTTCATCCAGTAGAACTTTTTGTCATCATGGAAATGTCCTATATTTGTCCTGTCCAATATGGTAGCTATTAGCC
ATATGAGGTGTCTCTGTGTGTCTGTGAGTGAGAGAGAGAGAGAGAGACAAGAGTCTTGCTCTGTCATTCAGGCTGGAGTGCAGTCGCGCGATCT
CAGCTTACTGCAGCCTCTGCCTCCCAGGCTCAGGTGATTCGACCCTCAGCCTCCCGATAGCTAGGATTACAGGCGTGGGCCACCACGCTCAGCC
AGTTTTTTTGTATTTAGTAGAGACGGGGTTTTGCCATGTTGACCAGGTTTGTGTCAAACTCCTGGCCTCTAGTGATTCACCTGCCTCAGCCTCC
CAAAGTGCTCGGATTACAAGCATGAGCCACTCCACCTGGCCTATATGAGGTTTTCAGCCCCCTTGAAATATGTGGTAAGTGCGACCAAAGAACT
TAATTATTTTATTTTATTTTATTTTAATTTAAATATAAACTAGCCAATAGTGGCTACCATATTGGACAGCTCTAGATAAAATTTAGGTAAGACC
CAAGAGATTGTGGTTTAGATTCTGCTTGGAATATGAGTGATTGGGGCTCTTGGAATTTAAAAATCCAAAGACACGTGTGAATTTGATGTTATGA
GTTTGAATAACAGCCTTCCTTCAGTATGTCTACATTGTTTTATCCTGACTGACATTGTAAGTCCTAAGAGGACTTACAACTGGTAAGTAAAGTT
GTGTTTACCAAAGCAGTTGGAAGTTCAGGCAGTTGTACGTTTTATTTGACCCCTTAGAGCTAAACAGGTGAAAATTTCAGGTCCTTTGGATGAA
GTAAGTTCTCATTTTATCTCAGCAACCCTATGAGGTAGGTTATGTCTGTTGCGTAGTTGACAAAACAGATGAACTTATATTTATGTACACCTTT
ATGGATTAGTAGCCTTTAAAATGGTTAGTTTTATCCCACAACTTCATTTGAAATTGTAAGTTTCTAGCATGTTGCCTATAACAAGAAGGTAATT
TATTTCTATTTGTTAAATTGTTATCAGTAATAAGTCAATATCCTACTATAAGTGGTAGAGAATCAAAGATTGTAGACTACCAAATAGCACCCAA
GAGGCTAATTCATGCAAGGTGTTGCTTATAGTAAAAGCAGATCAGAACTCAGCTGCTGTATGCCATCTGGGGTTATCTTCTCACTTGCACGCTC
ATGACCAGTGTAGGCTGCATCTTATAAGTAATCTGGCTCACAGTTAGCTAGACATATATTAGTCTGATGGAACAAAATCCGATTGTAGCTTTGA
CCACTATGAGGACCACTGTCATATAAGGTCTTAGATACCAACTTGAGTAAATTGCTTAAGGACTGGGAATAGATCCAGATTCCTCCAGGTTTAG
CTTCAAGTTGTGATCAGTTTTCGTGGGATGCAAGGAGAAAATGAATAGGAATGAAATAAATAGCTGATATTTGAACATTTTTAATGCCCTAGGC
AGCGTCTTAAGAATTCTAAGCTTTCATTTATCAATCCTGAGGTAGTTAACATTACTATTTCCAGTAATGTTCACTGGGCCAGTTTACTTTTGAA
GAAATTGAATCTCACGGTTTAACTAAAGTAACTAGCTCAAGGCTATATAGTTATAACTAAGCCACAGAGCAGGCAGGCTAATCTAGGCCTACCT
CATGCCACAGCCTGACTTCTTAGTTGTGATACAAAACAAGGTAAAGGGGGACCTGGGGTGACTTAGTTGCCTCCTGAGAATTCTGGCCTGTGGT
GGAATTACCCATCAGACTGGGCGCGCCCATCTCTTCACTTATGATGTGTCCTTAACAGACTAAATGCAGGCTCTGCTGCACTTTAATGTAATTC
TTTCCCTATTAAGGGAAGCTTGCTTGCCTTTGCAGCAAAGCCCCCTGGATAGTTTATTTTTTAGCAGTTCAGCTTGTGCTTGGACCCAGTTGTT
CCAGAAATCAGTAAAACCTATTTGAGTATTCAAGAAAAAGGATCTTTTTGTTAACTAAGGATTAACTAGAAACCTTATTTTATAAGGGTTCCTG
AATTTAACCAGCTTGCTGCTTAAGAAGCAGAATTAGCAGTCGCCCAGTCATTCGCCTCTCTTCAGAGTTCTCATGTGACTCACTTAGTTTTGCA
GAGTGAGGTAGATTTTAACCACTTTCAAGTCAAGTTGATTGGCCATTCATGAGCTGTGTGCAGGGAGAGAAGAGAGACGAGAACAAGTTAGCAG
AAAGCTGAGGTGTGTTAGACGGGAGTTCTAGGTAAGTCACGGAGGATATTCTTCATTTAGACCCTGACCTTGAGTAAGACACCTTTCTACAGGA
GACATTGCGGATACTTTCTAGTTAGTACTAGCGCAGTGTTCTGCAAACTACACCTGCTTTTGTGTGGTCCACGAACTAAGAATGGTTTTGTATT
TTTAAATTTAGGCATGTGCCACATAACAAGATTTCTGTCAACAATAGACCACTTATATAATGATGGTCCCATAAGATAGAAGGTGGTATTTTTA
CTGCCTCTTTTCTGTGTTGCAGATACGTTCAGATACACAAATACTTAACACTGTGTTACAGTTTCCTACAGTACTGTAACATGCTGTACAGGTT
TGTAGCCTAGGAGCAACAGGCTATACCATATAGCCTAGGCGGTAGTAGGCTATACCATCTAGGTTTGTGTAAGCACACTATGATGTTTGCATGA
CAAAATCGCCTAATGACACATTTCTCAGAATGTAGTTGGAGGAAAAAAATTGAAAGGAGAGTATTACTTCATGAGACAGGAAAATCATTTGAAA
TACAAATTCCAGTGTCCATAAATAAAGTTCACTAGAATGTGCCCATGCTCATTCATTTACATGTAATCTGTGGTAGTTTTTGTCCTACTGTGGC
AGAATTGAATACTTGCAGTGGAACTTGAATGGGCTACAAGGTGTATTTAGTACCTGGCCCTTCACAGAAAAAGTTTGCTGACCTGATTGAATGT
GCTTGTGTGTATCACAGGAAGCTCACCAGGACTCTTGGTATGTACCCTCTAGCTCCCAAAGGCATCTGAGCCCCACTGGCAGGACTCAGTTACC
TGTCTCTGTCTAAGCATGACTCCACCCATTAGCGTCCAGACAACGAAGAAAACTTCTATTTTAAAGAGCTTGACTAACCCCCTCCTTATTATCT
AAGTTAGAGTAGCTGTGCCACCCCTTAAGTAGCTTTTCCTTCTTCCTTTCAAATAGGGGTTGGAAGAGTAGCAGGTGAGAACAACCTCTTTCTT
CCTCAATTGTAGAGACTCAATAGTCTAGGCTATAAATTAAAAAAAAAAAAAAAAAATTCCCCAGAGGCTGACTCAGCACATGCCTGCCTTTTAA
AGGAACAGAGAGACCCAATTGGAATAGAAACTGAAAATAAAATCAGCAGCACATGAATTTTTGTAGGTGGCTCGTATTTGCTATTAATATTGCT
CAGAAGACTCAGGAATAAGAAATAATAATGCAGGGTTTCTTCAAAATATGGTTCTGGACAGTGGATTATAGTTACCTGGAGAGCTTGTGTTAAA
ATATCTGAGGATGATTCCAAGTACCAGGGCTTATACACAGGAATACTTGAGAACCACTGCACTCAAGCATTTAAAATTTTCCTTTTACCTAAAT
CCACATGGGCCAAGACAATTTTTAAAATTCATAGAATTCATGTGACCAGACGTAAGGGGTCAATAAAGGTCATTTTATAGATCAGAAAACTGAG
ACCCAAAGCTGAGAAATAAGTTATCTAAAACCCCACCAGGACATTGCATTAAAGGAGTTGCTCTAAGCGTGTCTTCTTACTATTAGAAAGTGAA
ATTCACTTTTAAATCTGTCACTTAGTTTTTGGCATAATAATAAAACAATGAACAGTCTGTTCTGGCTCTAATTGCGCCTAGAATCTCCAGATCA
GAACACCTTCTTGTAGATCAAGTAACATACTTCTCTGAAAGACAGTTTGTCGGTCCTATCACTGAAGTCTGAATTATTTTTTTGATTTTTAAAT
ATAATTAATTGGTCCCTGGAAATGAGAACCAGAGGAAGATAAGAAGATGAACTTGAATTTAAGGACCATAATGGAAGGAGTGGGATGGGAAAGA
AGGCTGTAAAATAAGACAGTGGTTGTGGAGAGACAGATTAAGGTAAGGAGAGAAGGTTGTAAAATAAGACAGGAAGGGGTTGTGGAGAGACAGA
TTAAGGTAATTGTGGTGTTGTAAAGCCATAAAAGAAATTTAAATGCGGAAGTGGCCTGGAAGAGAAAACATTCTCTGATCATTTAACCCTTTTG
TGTGTGTGTCAGTTTGAGGCGTAAAGTAGACGAATAGCCTCCTTCTTGTGTGTCTTTTCCATGGCAAAGCCTTTAAGGATAATAGCTGACCACT
AATCTCACACAGCTGCTGATGTTTAAGAACCTAATTCTTGAAGTGTTTTTAAGTCAGTTTGTGGCATTTCTTCTGAACACTTGGGCACTTTGGA
AATTGTAGTGACAGGTTTTAGAAATTTGAAGTTGTGTTGCAGAGGTGATAACAAAATTGGGTCTTTGAGCAACTCTGAGTCTTTTAATGCAGTG
CACTTCTAAGTTTCCGATGGTATTTCCCCAAGTTTTGAATATTGAAAAGAAAGACCACACTTTGCAAAATAATTGGCAACTTTCATTTAATTCA
GTTTGACCCTGTCTTGAACTTGGTCTTTGTAACTTCTTATGAGGGTCTTTTCCAACGGTAACATTAGAGGCTGATTCTGGAACTTCATGGAGGC
AAGTCCAGTGAAACTTGGGCAGTTAGGGGGACCCTTTCCCTCTCCCTTTTCAAAGTTACTCAGTCAGCAGCTGCTACTGTTGGGATGGCGTGGT
CAGGTGGGTATTATGCTTATTATGAAACTTGATGAGGGCAGGGACTCAGTTTTGGGGCTGAGTTGTATTTCAACACTTAAGTGGCACTCAGTAA
ATACTTGAACAAATGAATATTCTGAATCCCAACTTCCTTAAGCACAGAAGAACTATTTATACTTGTCTCCCATCTTCTTAGCTAGAGATTTTTT
TTTTGTTTGTTTTGTTAGCTTTCAAGTTGAGGTCCCTAACAGGGGAATCCCATAGTTTCAATGATGAACAGGTCCATGACAAAAATTCTTGTTT
ATATGCAATACACATTTAGGGAGAATAAATGGTCATGTGGACTTTAAAAATTGGAAATGTTACTAGTCTCTTCTCTCATTCTTTTGTTTTGTTT
TATGTTGAGGCAGAGTTTCACTCTTGTTGCTTAGGCTGGAGTGCAATGGCACGATCTTGGCTCACTGCAACCTCCGCCTCCGGGTTCAAGTGAT
TCTCCTGCCTCAGCCTCCCAAGTAGCTGGGATTACAGGTGTGCATCACCATGCCCGGCTAATTTTTGTATTTTTTAGTAGAGATGGGCTTTCAC
CGTGTTGGTCAGGCTGGTCTTGAACTCCTGAACTCAGGTGATCCACCTGCCTTGGCCTCCCAAAGTGCTGGGATTACAGGCATGAGCCACTCCT
CCCAGCCCTCATTCATCAGTTTTTTAAGGGTTTCTTTAGAGCTGAATCCTCCCAGCAGTTTTCTTTCCTTTTGAATTCCAGAGCTCTTTGGGCC
TTTAGCCAGAAACAGTCTCTGTCCTGCTTACTTTGAAAGGCTGTTAACTATGCCCTGGAATTTTGGAAATGTTCTCCTTGAGAATTGCTGTAAA
GTAGTGATAAGGACTCTTCTGTAGGCTACAAATTGAAACCAGCCACCATTAAGATGGCTCACAGACAGGGTGTCCTGGATTGAAACTGGACCAA
CGTTGGTATAGTCTGAATTTGAGGAGTAAAAAGAAATATGCCCTTCCCTCTCCCCTAACAGTTGGGGAATACTTGTTTTTACTCCTCAACCATT
CCCCTTTTAATGATGACAAGTTTTCAGGCTTTTTCACTGCCTTTAGTTTTGCATTAAGTTCCTGGGAGATTCACACACCCCTGATATTTACTGC
ACTCCTGGATGCCAGTTTTTTATCACTCAAGACCAGCTTGTTATCTGTCCTAGACAGAAGCCATCTTCTTTTAAAAGTATTAGAACATACTGCC
TATAGAGTGTTAAAGAGAATGTTGATTTTTTTAAAACTTCTGCTTTTGGGGATGTGATATGGGTTTGAAAGTCAGGGAGGAAGTAGAAGATGTA
AGGGTTTGCCCCAGTACAAGAGTTTTAATAATGAGGTGATAAATATTTTCTTTAAATAATTTATTCACCCATTGTGTTCACTCAAGTGGTTCTA
GTGTTCACTTTCCTTATGCGCTAGTTGTATATTTGTATGTCAGAGAAGAGCAGAGATTATCTTTGACAGAAGTTATATTGTGAGTGTCTTTTTG
GAGAAGGTAGGTTTTGATTGTGTAACTATGCCACACCTCCATTCCCCATACACTTGACTTGATGATTATCTTTGAAGGAGGAGCCCATCATATT
ATACAGCTGCCCTAAGTCTCAAATCTATCATCAAAAGGCAGTGTAAAGGGGACTGGCTCAAAACATGGCAAAGAGTCTCCTTTATATCAGAAAG
CTCTCTGCCTTTGCTTATAAGGCAGCAAAGGACTCTTCTTCCTGCCCTGATCTGATTACCCTAAAAGCTTTAAGATCATGGAATAGTTGATAAA
ACAGTCAGTATTCTGTGCTGATGACTTATCCTTTTCTCCCTTAAGAAAGAGTAAATGTTTTTGCTTGGATTGAGTTTAGAGACACCAGATAGAA
TAAGATTGGCAACACTGTTAGCCACCTGCTGACCACAGCTAAACAGGCATATGTGCTTCTGCATGGGGAATGCTATGGCGTTAACAGTGCCTTC
CTTAAAACCTGTAAGTTCGTTAGCCTTCCGGGTACCAGGGTTGTTAGAGTAGGCCACAGTTTACTTCCTTTTTCATAAGTGCTTTTCTTTCCTG
GATCTCAGTATAGGAAGGAAAATCAGACAGCATTTCTCAGTTGTTGGCTCGGTGGGCATTGTGAATGGGTATGGTTGCTGCCTCAAATGCACAT
ATACAACCTGAGCTAAGTTGTATGAAATGATTGAGTAGGCTGACTTCAGTTCTATGTAAATTAATCAAGGAGAAGGTTTAGGAATGGTTGTACC
GCATTGACAGTTGTTAACATGTTTATAATTTCTTGGAGATTAGCCAGGAATTCTGTTACAACTACACACAAATATTGATTGACTTTCAAAATAA
AATTCTTTAAAATCACTGAAATTTTCTGAGCCCAAGTTTCTTCATCTGTAAGATAAGCAAATTTACTAAGGTCATGGGTATCTAGTACATGATG
GACAGAGATTTGAAGCTCTTTACCACTGATTTATGCTACCTTCTCATAGATCTAGTAGTTTAGTGACTTTTACCCAAGGTCATACACTGAGTGA
TAGAACAAGGATTTTGAATAAGATATTTTTCACTGTTGCACTGGTCTGCTTTCTCCAGCTTCCTTCAGAGGTCCCTAAAGGGGACCTCTGTGCT
GCTCTGCAGTGCCTCTATCACATTACGGTTTGGTCATGGTCTTTTGTGCTCATCATGTTTGAACATGTTTATCAAAGTATTTTACAGACTTGGT
TATTTCTTCCCCTCATCTAATTTGTGTATTTGTCTGTTTTCCCTATGGAGAATCAACTTTTTATCTTATATATGCCTGATGTGACATTGATTTG
GCCACTTCGGCTTGACCTTGTACTTGCTTTCCATATCTGCAGCTTCATTGTCATTTTGGGTTTAGGCCAGCTTTCAGGAACTTTTTAACAGTTG
GATCAAATTTTGTTGGATTCATTTGATACTGAGTTTCTTATTCCTGTATATTTTGTTTCCTTTCTATCTAAGCAGAGAACTAGGACTCTAGAGT
TACAAGAAGTTAGAGTATATCTATAACTTTTTTAAAAGAAAAAAAAAGGACCAGGCATGGTGGCTCACTCCTGTAATCCCAGCACTTTGGGAGG
CCAAGGCAGGTATATCACTTGAGGTCAGGAGTTCGAGACCAGCCTGGTAAACATGCTGAAACCCTATCTCTACAAAAATACAAAAATTAGCTGG
CCATGATGGTATCCACCTGTAATCCCAGCTACTCGGGAGCCTCAGGCAGGAGAATCTCTTGAACCTGGGAGGCGCAGGTTGCAGGGAGCTGAGA
TTGCACCACTGCACTCCAGCCTGGACAGTAGAGTGAGACTCTGTCTAAACAAACAACAAAAAAAAACTCTGTTTTGGACACATGACTTTAGGGA
GATAAACATAACTCTGGGATTTTATTTTTTAATATGCTCCTGAAAACAGTCAACTCTCCCATTTCCTACCTGGGAGAAGTATGTCTCACTTAAT
TTTGCTCATTCAGGAGAGGATGAATTTTTCACCAATGATAGTAATAGCTGGCTGTTGCAACTCATTTCTTGCTGCAGCGTACCACATCCACCAA
GAATGCCCTTTCACCTTCCTTTGTCTTCCCAACTCCCTCTTAGCTTTCAGTAAGACTTGGCTCAAACTGCATCCTCCAGGAAGCCTATGTTAAC
TCCTAACTGCCACTCTTGGATCCAGGGTTTTTATTTCATCTTGTTCGCTGGTGGAACCGGGCTGTTTTATCTCATTATATTACTCTTCACTTTG
AAACAAGGAGGCAGTTAACCTAGATTCAGTATTTTTACTCTTAGACTTTTTTTCCCCCAAAATGCCCATAAATTAAGCAATCTTACTGCCTTAC
TGCGCTTACTGAAAAATAGCAGCACAGCAGAAAAATAGAAAATCTTGCTATCATGTAAGAGATGTGGAAGTAATGGTGCCCACATGCTTCATTT
GGAAAATGTCTATTACCTTTATAAAGAGTTTGTATAGTTTCTAAATACAGGAGCATTATCATGCGATAGAAACTGTTACTTTAATGTTAATTGA
CGTTCGAAAATTTGAGTTTCAAATGTTTCCTAAAACATTTTTTCTTAACCCTTTTGCTATTCCATTGATCTGCCATTCTACATTCTGCCTCCTT
TGTTTCCCTGTCACTTATTAAAAATACTGCCTCTTAACTTTGTTGTCATCCACTTATTTCTCTAGATACACCTTGGAGTGTTTTTTCAGGCTGA
ATTGTTAGGCATCCGATTGCTGGGTTTAAAGACGGAGTGTGTGAGTTTGTTTGATTTTGCCAAATTCTTCCTTCATGGGGGTTATACCAACTAG
TATATGTAATACTGCCTTGCACAAAATATACATTTAGTAAAGGATACTGATATGGTTTGGCTGTGTCCCCACCCAAATCTCATGTTGAATTGTA
GCTCTCATAATCTTCCTGTGTTGTGGCACGGACCCGGTGGGAGCTCATTGAATCACCCGGGCAGTTACCTCCATGCTGTTCTTGTGATAGTGAG
TTCTCATGAGGTCCGATGGTTTTATAAGGGGCTTCCCCACCCCGCCCCCGCACCTGCCCCACTTTGCTTCACACTTCTCCTTGCTGCCATCATT
TGAAGAAGCACGTGTTTGCTTCCCCTTCCACCATGATTGTAAGTTTCCTGAGGCCTTCCCAGCTCTGCAGAACTGTGAGTCAATTAAACCTCTT
TATAAATTACCCAGTCTTGGGTATGTCTTTATTAGCAGCGTGAGAATGGACTAATACAGATACCTACATACCCATTATTATCTTTTGTGAACTG
GGACTATTTCTCTATCGTAAGAGTGGTAAGAAACTTGAGTTGAGATAGTGTTTGAAAGCACGCTAGCATGGTCCTGGCACCCGTATTAGTGCCC
TTTTCTCTTGTGTGCCTTGGGCAGCCAGCTACTTGAGATTATATCTTAATTACCTTAATGGATTGTAAACTCCTTGAGCGCAGAGTCCTTGTCT
GGTGAGTCTTTAGCAGGAAGCTTTGGACTTGAATATATTTGGTTTTAAATTGAATTAGTCTTCAGAATATATTTCTGAAACAGTAGGAAAAGGA
TTTCTTTTTTTTCTTTCTTTTTTTTACACATTAGCCCTCAACTCCTGTGTAATGCTACCTAACATCTATTCCTACAGTAGGAAAAGACAGAGAC
ACTTTCCTTTTGTCTGTCGGAAGCTTCTTTACTTAAAAGCTTTTTAAACCCCAGGGAATACGTTAGGTAACAATAGCAAAAGATGTTGATTCCT
GTTCTATCTGTTTAACTTATCTGTCCTTAGGGAGTAACTCCAGTTAAATTACAGTGAAAGGAGTTAGTTGCCATAAGTCTTCTGTTTTATCAAG
ATTGCTTTATTGCAGGCGGCTTCCTCCCATGTCTTCATCTAGAATTTTCACTAGGAATTAAATAATTTGTTACTAGAAAGCATTAGTTGCCCTG
AATTCACAGTGAATTAGGTTTTCTGATGATTTAATGTGCACTGGTTTGCATCTTTCATATCTGTCAGTTAAATTCAACACATTTCTTTTTGCAC
TTAACAGGTGCTTGAAGTGGCAGGGTCCTTCCTCTAAAACATTTTAAATAACACTAGTAGATGAATTCCAGTTAAGCGGAAAAGGTATGTGCTG
AAACTAATCTTCAAGGCCACAGTATGTTCTCTCCAAACACATGTTTTCACCACCAGGGATGTAGATCTGTGTGCCCTTATTGGCTTTACTTTTT
CACTTACTGGCATCTCTTCTGACCAACATCTTTAGTCAGATGTCCCACTGTTACCTTAGATACAATATGGCTCAGGCTGGTCCATCATTTCTCC
AGTATTTTTCACCATCTTTATTCTGTACAAGTTTCTTGTTTTTGCCAAGGCCCCTTCTTCCCCCAGTCACCTGGACGTAAAGACTTTCTCTGAC
TCTTTATTACACTGCATTTATTACACATAGTAGTGAGTTCCTGGAACTGGGTTCTACTTCCATTTTCCCAAATGTGCTTTTACATTTTTTCCTT
CCTTCTTAGTTCCTTTGCTGTCACTTTTTTTGTAGATTGCTTCAGAAACCTCTAACTATTTATTTTGCTTTTACTTTTATCTCTTACTATGCCT
TAACACAAACCCTCTCTTGGAACTAGGCCAGTCTTCTTGCCATGCCCTCACATAATAACACAAAAATGAAGGGTTGGAAAGGAAGAAGCTTAAA
GATAATGTTTAACTTATGAAAAGTTTCAAGCATACACATAGAGAAATATAATGAACTCCCATGTGTCTGTCACTCAGCTTCACTAATTACCAAC
CTTTTGCCAGTTGAATTTTGTCTATCCCCGTATCCCTTGGTGCTCCCCCAACCACCACCACCAGAGAGTATTATAAGCAAATTCCAGGCATCGC
TTACATGGAAGTACTTCAGTATTCATCTTTACAGATTCTTTAATCTGTCAGTTTACCTACTGCAGAATATCTCTACAGTTTCCCTTTCAGTTGC
TTATTTAGTTTGTTTAAATGCTTTTGGCGTTCAGAAGGCATCCCATTCTTTTAGTGGTTAGTTCTTTTGTACTGACCTCAATTTGCCTCCCTAT
CATTTCTCAGCAGTAATATAAATTTATATTTACTCAGAAATTACAAATTTACTGAGTAAATTTATATAATACAATATAATAGTCATTAAGATAT
TTGAAGAAGTTGCCATGTAATACACTTTTTGACACACCTTTCACAGCACACATAGCACATGTAATAACTTTTATTTGTGTTTTAGAGAGAGGGT
CTTGCTCTGTAGCCCAAGTGGAGTGTGGTGATGTAATCATAGCTCCATCCTTGAACTCCTGTGCCCAAGCAGTCTTCCCACCTCACCCTCAGAG
CACCCAGTATTACACGTGCACCAAGATGCCCAATTAATTTTTTAAACACTTTTCGTAGAGACAAGGTCTTGCTATGTTGCCCAGGCTCGTCTCA
AACTCCTGGCCTCAAGTAAGCCTCCCACCTTCGCCTCCCAAAGTGTTGCCATTGTAAACATGAGCCACCGTGCTCAGCCATTCCTTTAAAATAG
GAATACTTCGGGTTCAGATCTTTGACCTAGGTCTTATTTGTATCAATGAGATTAATGAGCCACAGCCCCGAGTTTCTCACCAGACATTTTTGAT
TTGCATTTTTAGACAGCAGTTGCTTTAAAAAACTTGGGGACCTGGCTTAAAATCAATTTTGAAATTTTGTGCATTAAAAAAAAAATGTGGCCGG
GCGCAGTGGCTCACGCCTGTGATCCCACCACCAGACTTTGGGAGGCTGAGGCAGATGGATTGCCTGAGCTCAGGAGTTCGAGACCAGCCTCGGC
AACACGGTGAAACCCCATCTCTACTAAAATACAAAAAATTAGCCGGGCATGGCGGCATGCACCTGTAGTCTCAGCTACTCGGGAGGCTGAGGCA
GGAGAATTGCTTGAACCCGGGAGGCTGAGGTTGCAGTGAGCCGAGATCACACCACTGCACTCCAGCCTGGGTGACAGAGTGCACCTGTAGTCTC
AGCTACTCAGGAGGCTGAGGCAGGAGAATTGCTTGAACCCGGGAGGCTGAGGTTGCAGTGAGCCGAGATCACACCACTGCACTCCAGCCTGGGT
GACAGAGTGAGACTCTGTCTCCAAAAAAAATAAAAAAAAAAAAGTTATTGGCAGACTGTTATCTGAAACCTGCCTGGAATTACTTTCAATTTAT
CCTTTACTTAATTTCCTTTTTAAGTTTAGAGCTTTTTAGTGATTTAACCAAATTTTCCTATGTACACTAGTATTTGTCAAGATAAATTTTTAAA
GCTTGGTCCATTTTTTACTATAAAAGTAATTGTGGGGAAAACTAGAAAACAAGATGAAGTATATGCACAAAAACTCAAGTTACTCATAAATTGA
CTACCCTAAGATAACTGCTTTTAATATTTTGATGTCTGTATTCCAGTCTTCTTCATATGTATTTATGCGTTTGTTGAATTATACACAGAGTAGT
TTTTTTTAATGTGATGTCCTTTCCATACTCTAGGGCTGTATTAGCAGATCTTGATGTTCTGAAGCTTGTTTATTGCATCTGGCCTGCAAACAAA
ATTAATTGACACACATGCTTTATTCCAGATTGTGGTGCTATTACATGTATTGTCATTATTATGATTTTAGCCATAAGTGTTTGGTTTCAGGCAG
AAGTAGTGGGATATTGCTGTCAACTAAACTGGGGCCACTAACCTTGTGGGAATCAACTTAGCATTTTAAAATTATAGACTCGAGGTTTTTAGAA
GAAGCTGAAGGGTTCTGTATGCTGCATGGAAGATTGAGTTCAAATCCTACTTCTGACACCATATGATATTATTTTTCCCATCTCTGTAGGATGG
GAATTTCTTACAGTTAGTACTACGGTCATGCAGATTTCTATTTTCCCAGGGCCTGAGACAGAATAGATGCTCAGCACATTATGACACATGTGGC
TTCTTTTGCTGTGAGCCAGTCATGGGATGAAGACACATTGCCTATGCATTATTAATTGATTGCTATAAAACATGTTGCCTCAAAATATAATAGC
TTAAAACAACACACACTTATTTTCTCATGTACTTCCTGTGAGTCAGGAATCCAGACATAGTTTATCTGCGTGGCTTTGGCTTACCATCTGTAGA
TTGCAGTCAGTATGTCAGCCACCGCTGCAGTCATTTGAAAACTTAATTTAGGCTAGAAGGTCCACTTCCAGGATGGCTCATTCACATGGCTAAT
TGGCAGGAAGCTTAAGTTTCTTACTACATGGGCCTTTCCAGAGGGCTTCTGAGTGAGTACATGACAGTGTGTCACTGGCCTCCCCCAGAGCAAG
GAGAGAGCAGGAAGGAAGATCTAATACCTTTTATGGCCTAGTGTCGGAAGTTGCCCCCTCACAATTCCACTTCATCTTGTTTGTTACGACTGAG
TCACAGTGTCCAGCCCACACTCAAGGGGAGGGGAATTAGGCTATACCTCTTGAAAAGCGGAGTAACAAACAATCTTATAGTCAAGATTGTGTTT
GTGTAAGGTTAGAATAGAGGGTCCGGAAATAGACCTATAATTATATGACCGCTCGAAAACGCCAAGACAATTTAATGGGGAAAGGGCCATCTTT
TAAAAAATAATGCTAGGCTATTCACAGTAGCAGAAACATGGAATCAACCTCAATTTTCATTAATGGTAGACTGGATTAAAAAAAAGAATGTGGC
GCATGTACACCACCCAATACTGTGCAGCCATAAGAAGAACGAGATTGTGTCCTTTGCAGGAACATGGATGGAACTAGAGGCCATTACCCTTAGC
AAACGTAACAGAGGAACAGAAAACCAAGTATTGCATGTTCTCACTTATAAGTGGGAGCTAAATGATGAGAACACATGGACACATGTAGGGGAAC
AGCAGATACAGGGCCCTTTTGGAGGGTGGAGGGTGAGACGAGGGAGAAAATCACGAAACATAATTAGTGGGTACTAGGCTTAATTCCTGGGTGA
TGAAATAATCTGTACAACAAACCTCCATGACACGAGTTTACCTATATAACAAACCTGCACATATACCCCTGAACTTAAAAGTTAAAAAAAAATG
GTGTTAGAACAACTTGACGTCTTTATTGATTAGAAAAAAATGAACACTGGCCCTTAACTCTGCAAAACTGCTAGAAAATAAATAAATAAATAAA
TAAATAAATAAATAAATAAATAAATAAATAAGGTGGTGGTGGTGATATGCCCTTGTAATCCCAGCCATTCCAAGGGCTGTGGCAGGAGGATTGC
TTGAGCCCACGAGTTCGAGAGGAGCCTGCGTAACATCGTGATATAAGAAAATGAGACTCTTTCCCTGCCTTTCCCTGCAAAAGAGAGAGAGAGA
AAATCTGTGTAGCCCTTGAGTAGGCAACGATATCTTGGGGCACAAAACATACAGACCCTAAAAGAGAAAATTGATGAAGTGGACTTGATCAAAA
TTTAAAACTTTTGCTCTTTGAAAGACACCATTAAGAAATAAAAACAAGAAAATGATGGGGAGAAAATATTCACAGTTCGTATAGCTAACAAAAA
ATTGGTAATCCAGAATGTATAAGAGCTCTTACAACTAGGTAGTAAGAAGACCAAGAAAAGATTTGAGCAGATACTTCACCAAAGAAGGTATATG
AGAGGCCAACAAGCACACCAAAAGATGCTTGTCATTAAACATCATTTAATCATCAGAAATATGTGAATTGAAACCACAGTGAGGTACCACTCCA
CACGTGCTAGAATGGCTAAAATTAAAGACTGATAGCAGATATTGGTGAAAATTTCTACCAATTGGAATGCTAATGAATTTTTGATGGAGATATA
CAATGGTACAATTTGGCAATTTCCTCTAAAGTTAAACATACATTTTTTATATTACCCACTAACTTTTCTCCTAAGTATCTACCCAAGAGAAATG
AAAACATGTCTATAGAAATATTTGTATATGAATGTTTGTAGCAGATAAATAATAACCAATGCCTGGAAACAACTAAATTGTCCAGCAGGTGAAT
GGGTAAGTTACAGTATATCCACACAATGTAACACCACTCAGCAATAAAATGGAACCAACAACTGATATATCCAACAATGTGGATGAATGTCACA
AATGTTATGCTGAGAGAAAGACATCAGACACAAAAAGAATAAATCCTATATGATGTTATTTACATGAAATCTAGAAATCTAGACAAATCTAATA
TACAGCACGATCAGATCAGTGCTTCCTTTGGGCAAGGGGTGGAAGATGGGGAATGACTGGGAAGGAGCACGAGGGAAACTTTCTGAGATGATGG
ACTTGTTCTAAATCTTTATTCTAGTGGGGATTGCACCTGTGGGTGTCTTTGAAATGTTTATTATTACAGTTATTTCTTAATATTTGTAGGGGAT
TGGTTCCAGGACCACCCCACCCCCTGTCCCTACTATACCAAAATTTGCAGATGTTCAAGTTTTTTTGTATATAAAATGGCACAGTATTTGCATA
TAACCTACACAATCCTTATATATACTTTAACTCACCACCACATTACTTAGATTACCTAATACAGTGTAACTATATAAATAGTTGTTATATTGTG
GGGGGATTTGTATTTTTTTGGTTGTATTGTTATTACTATTTTTATTTTTCCTTGAATATTTTCAGTCTGCAAATGGTTGAATCTATCGTGCTGA
ACCTGTAGATATGGAGGGCTGATTGTATTTTATTGTTTAGACATTACACCTTATTAACATTCATTTAAAAACATCTGTTGAGGTTTGGATTTTT
CAGAAGTTATAACATCTGATCTTTAGAGTCAGTGTTCAATACTATCCCTTTGTTATCATTTATAGGTCAGGCTTAATAGTATTATGTCTAGGGA
GAAAACGCCAAACTCTGGCTTCTTTCCACAGAATAATTGCCTCTCATTTCCTTTATCCTCCCCATATTAGTAACTTGTCTTGATTGTAGAAGCA
GGCTAATGGGAGCTTTGTCTGGGCTTCTTTGTGAAAGCTTTTTTTTTCAGTGGCATGTGCTATTATGCCTACCATGCCATTTGAATCCTGTCAT
TGTACTTCTTGACCAGCTTGTCAACGTAGGCATACAGAAGATAGCTCATGACTCCTGTTAACTGACTCATCTCTTATGGTTGCAGATTCTCATA
AACACAAAGATAAACACAAAGATCGAGAACACCGGCACAAAGAACACAAGAAGGAGAAGGACCGGGAAAAGTCCAAGCATAGCAACAGGTAAGG
GTGGAATCAAGCAAGTCCCTCATCATTCAGCATTGGGTTGGCCATTGCTTGGCTTACCTGGAATAGGTCTTAACTTGAGCTACATGTAAAGATA
GCAAAGTAACTAGAAACTGTATTTGATCCTAGAGTTCCTATGAGGATGGCCATGATTTAGGCCTTAAATGGGATGCCATTGTTCAGATCAAGAT
GTATAAAGCAAGTATGTTTAATTTTCTTTTAATATATACCTCATTAAAAATACAGTTGTTCACTAAGATAGGTAAGGATTCATTGTGACACATG
ATGGCATTTAATCAAGTCTGTAGCAAGCTTGCCTTGATAAAAGGTAACTGGATTGGAGACCTGATTTTTTTTCTTCTTTCTATATTTTAAAGAG
TTTACATTAAAAATTCTTAACTTGGGCTTCCATGGTCTGTTAGCCCCTGAAATTATATGCAAATGGTTGAGTATATGAGATTTCCTGGAGAGAC
TATTTGTAGCTTTCATCATGTTATCAGAGGAGTTCGGCTCCCACAAAAGCCTACAATCACCAATATGAATGAAACTCAATAGTGCTGCCTTTTG
GGATGCAAGCCTCAAGAAGATGTTTCTTTTGTTCATTTTGCTCCCATTTTTTTGGTGTACGAGGCAAGTGGTTAAATCTAGTTATTGAAAATGA
GGACTTGTACTTCCATTCTGATTTAAAGACAGTTGTTGGTGAGAAAGCCATATGGTATTCCCATTCCCATAGTGGCCTGAGCTAGGTTAGGGAA
TGTAAACATCTGTCACACATTACAACAAGTGTCTCCATTGTAGAGTTCTTAGAACTTCTTTATTAACTTGTCTCATTGTCTGTAGACAAGTTAA
AGTGAGAATTGCTTTCTAAGTCTGTTCTTTTTCGCGAATCATTAAAATAACCAGATTTAATTTTCTCTATTGGGGACATTTCTTTGGGAATCAT
GTATCATGCCTGTGGCCTTTTTTCCCTATCCCTCACTTATCTCTTATCACTTGATCATCTAGTTTCCATTTTCATTATTCTGGTAAATTTAATC
TCAGTAGTTGAATCTACTCTACATCTTTTCTAGTTTATAGTTGTCTAGCTTATGTTAATATCTACCGTACAGCAAAGAATATTGCCAAGGATAA
AGAAGTTCATTGCATAATGATAAAAGGGACCCATTAGGAGAACATAGCAGTTATAAATGTTTATCCACTAACGTCACAGCCTCAAAATACATGA
AGTAAATCTGATAGAGCTGCAAAGAGAAATAAACATTCATAATTATGGTCAGAGATTTTACTATCTTTCTCAATAATTTTTAGTCCAAATAGAA
AATAATTAAGGATATGGAAGACTTGAACAGTATCAACCAACTTGATCTAATCGACATTTATAGGTTTTTCCCATCCAACAATATTAGAATTTTC
TCTTTTCTTTCTGTCTACTTTTCCGCTTTTAATTTTTTTCAGGTTTTATTTTAGATTCAGTGGGTACATATAGGCAACTTTGTTACCTGGGTAT
ATAGCTGTGATGCTGAGGTTTGGGTACAAATGATCCTGTCACCCAGGTACTGAGCATAGAACCCAACAATTAGTTTTTCAACCCTTACCGCCCT
CTCTGCTCTAGTAGTCTCCAGTGTCTATTATTGCCATCTTTATGTCCATGAGCACCCGATGTTTAGCTCCTGCTTAAAAATGAGACCATGTGGT
ATTTGGTATTCTGTTCTTGCCTTAATTTGCTCAGGATAATACCCTCCAGTTGCATCCATGTTGCTACAAAGGACATGATTCAGTTCTTTTTTTA
TCACCGTATAGTATTCCGTGGTAGATATGTACAACATTTTCTTTATCCAATCCATCATTGCTGGGTACCTAGGTTGATTCCATGTCTTCCCTAT
TGAGAACAGAGCTGCAGTGAACATGTCTGTACATGTGTCTTTTTAGTAAAATGATTTGTTTTCTTTTGGATACATACCAAGTAATGGGATTGCT
CCATCAGAAGGTACTTCTTTTTTTAAGTTCTTTGAGAAATCTCCAAACTGCTTTCCCTAGTGGCTGGACTAATTTACATTCTCATCAACAGTGT
ATAAGTGTTCCCTTTTTTCTGCAGCCTTGTGAGCCATCTGCTGGTTTTTGATTTTTAATAGCCATTGTGGCTGGTGTGAGATGATATCTCATTG
TGGTTTTGATTTGCATTTCTCTGATGATTAGGGACGTGGAGCATTTTTTCATACATTTGTTGGCCATTTGTATGTCATCTTTTGAGAAATGTCT
GTTCATCTCTTTTGCTTGTTTTTTAATGGGGTTACTTGTTTTTTGCTTGTTGAATCATTTAGGTTCCTTATAGATTCTGGATATTATACCTGTT
AGACGCATAGTTAGTGAATATTTTCTCCCATTCTGTAAGTTGTCTGTTAACTCTGTTGATAGTTTAATTAGATCCCATTTGTCAATTTTTCCTT
TTGCTGCAATTGCTTTTGGCATCTTTGCTATGAAATCTTTGCTCGTTCCTATGTCCAGAATGGTATTGCCTAGGTTGTCGTCTAGGGTTTTTAA
CAGTTTTAAGTTTTACATTTAAGTATTTGATTCATCTTGAATTGATTTTTGTATGTGGTGTAAGGAAGGGGTCCAGTTTCAGTTTTCTGCGTAT
GGCTAGCCACTTATCCTAGCATCATTTATTGAATAGGGAGTCTTTTCCCCATTGCTTATTTTGGTTGACTTTCTTGAAGATTAGATTGCTGTCG
GTGTGTGGTTTTATTTCTGGGTTCTCTGTTCTGTTCCATTTGGTCTATGTGTCTGTTTTTGTACCATTACCATGCTGTTAGTGTTTCTGTACCA
TTACCATGATGTTAGAATACATTCTTTTTAAGTGCACACGAAATGTGCCAAAATAGCTCATACTTTGGACCCCACTCTCCTTAAACCATCTTCT
TTGGCTTTAATGCTTCCATGCCTCTCCATCTTTCTGGCCACCTTTTTCACCTCTTTCTAATGCACAGTCATTCCTTAACAGTTTTTGGCTGTCT
TTTCTCTGCTACCTGTTTTGCTGTTTATCCTTATTTCACATTCCATATTTGGTCTTGTGAACTTCTTTTCAGTTCCTTAAACATAGCATGTTTT
CTGATGCCGCTAAGCCTTTAAACATGCTGTTGGTTCTGTAATACTCTCTTGCCCCTTTGCTGTCATTCTTTTAGTTGCAGCTCAGACTTGACTT
GTTCCAGGTATCCTTTCTACCCTCCTTCTCATATTAAATGCCACTTTTGTGTGCTCTCTGGTGCCTTGAATATATTCCTAGTGTAATAATTATT
ATATTTAATTGTGTCTTTACTTTTCCCTATGGCCTCCTGGACTCTAAAGTCCCTGAAAGCAGAATTGGGATCTTTTTTGTTTTTTCTGGGGTGG
GGCGGGGGGCTGGTTTTTTGTTTGTTTGTTTGTTTGTTTTTTGAGACGGAGTCTTACTCTGTTGCCCAGGCTGGAGTGGACTCGTGCCATCTCA
GCTCACTGCTATCTCCACCTCCCAGGTTCAAGTGATTCTCCTGCCTCTGCCTCCCAAGTAGCTGGGATTAAGGTGCGTGCCCCGACACCCAGCT
AATTTTTTGTATTTTTAGTAGAGACAGGGTTTCACCATGTTGACTAGGCTGGTCTCAAACTCCTGACCTCAAGTGATCTGCCCACCTTGGCCTC
CCAAAGTGCTAGGATTACAGGTGTGAGCCACCACGCCCAGCCTTTTGTATTCATTTTGCATCCTTCAGGCTTAGTATGGTACCTCGCAGATAGC
TGGCACTTACTAGTCTATATTTATTGAATTAATGACTGGATGATCTATGGGTACATTAATTCCCATCTCTCTTTCTTCTCACGTACTCTTAATC
TTCTATAGGATAGTTATTAAGTCACTTTGGCTTATCTCATTTTTCACACAATTGTGTGTGGTAAAATAAACATAACAAAATTTACTGTTTTAAA
GTAGCATTTAGTACATTGACAATAGTATGCAACTGTCACCACTATCTCCAGAACTTTTCATCATCCCAGTCTTTGAATTCACTAAATAATAACT
TTTCATTCTGTTCCCCTGTCCCAATATCTTTCTACTTTGTCTTTTTTTCTCTTTCCCTTCTACCTTTTATCTTTATCAATTTGCCTATTCTAAG
TACTTCATATAAGTGGAATAATACAATATTTATCTTTTTTAAATCTGTTCTATATTCCCTTAATATGTTTTCAAGGTTTATCCGTATTGTAGCG
TATCAGAAATTCATTCTTTTTTATGACTGAATTATATTCCATTGTATGCCTGTAATCACATTTAAAAAAATCCATCTGTTGATGGACACTTGGG
TTGTTTCCACCCTTTCCCTACAAATACATGTTTGAGTTCCACTTTCATTCTCCGGGGTCTATACCTAGGAGTAGAATTGCTAGATCATGTCCAA
CAATGGGTGGCTGCCTGCTATCTTTTTACTGTGCTAATTGATGTGGTGTGAAAAAAAAAATTAGATAACCTTTGACCGTGAACAGTATCATCTG
AGGAGGTGAGAGACCTGCTTACAGATCAGATGAAGAATGTTGATTTTTATATTACTAGGCTGTAAGAAGAAATTCCAAAAGACTTTAAGAAGAA
AGAGGTTAATATTTAAAACATAAGCTTTTGAAAACTTATCTTCTCTAACACATTCTACTTTGTTTTGGGAGGAAAGATACAGTAAAAGTGACAT
AAAGGGCTTTGAGACATATAACTTTGGCCCTGTCCTGTTTTACAAAAATCTTCTCTTAAAAAAAAAAAAGGGATGTGATTTTTTTAGAAGAAAT
CCTCATCCTTGTACATCCATTTAAGTCTCGTGATCTTGGCTACGTTACTTAACCTTCTTCAACTCAGTTTCTTCATCTTCATCTTGATACATGC
TTCACAGCGGAACCATGGGACTCAATAAATATACAAGCTCTTCAGTTGTGAACTGCTCTACAGATGTGCAGAGATGACACTTTTACATCTATTT
AAAAGCATACTTTTCCAAGATCATATTGTAGGTAAATTCAATAATTTGTTGTATGTACTTTTGTGCCTCTTTTCTTTTCTTTTTTTTTTTTTTT
TTGAGACAGTCTTGCTCTGTCGCCCAGGCTGGAGTGCAGTGACACAATCTCGGCTCACTGCATCCTCCGCCTCCCGGGTTCATGCCATTCTCCT
GCCTCAGCCTCCCGAGTAGCTGGGACTACAGGTGCCCGCCACCATGCCCCGCTAATTTTTTATATTTTTAGTAGAGACGGGGTTTTACTGTGTT
AACCAGGATGGTCTCAATCTCCTGACCTCGTGATCCACCTGCCTCGGCCTCCCAAAGTCTTGGGATTACAGGCGTGAGCCACCGCGCCTGGCCA
CTTCTGTGCCTTTTAATATTTTCTTCATTCCTATAAAAATAGAAGCCTTTTTGCCAAGAGCTATTGGAAAAACCTGGCCCGAGTTTCTGTTTAC
ATATGACATGACTAAATCTAGCCCTTGGTTTCTTATTCACATGAGTAAATTTTAACAGTTTAATCAGATGATCTGTTGTAAGCAAACTTTTAAG
TACTTCTTGTTTTGATGTCTACTGACACAGTACCTCTTTGCAACAAAGCAAGGATATTCGTTATATTCCAGTAAAATTATTTTATTATTTTTTT
TGAGATGGAGTCTCACTTTGTCACCCAGGCTGGAGTGCAGTGGTGTGATCTCGGCTCACTGCAACCTCTGCCTCCTGGGTTCAAGCGATTCTCC
TGCCTCAGCCTCCCGAGTAGCTGGGACTTACAGGCGCCTGCCACCACGCCGAGCTAATTTTTGTATTTTTGGTAGAGACCAGGTTTCACCATAT
TGGCCAGGCTGGTCTCGAACTCCTGGCCTTGTGATCCTCCTGCCTCAGCCTCCCAAGGTGCTGGGATTACAGCCTTGAGCCACCACACCCGGCC
TCCAGTAAAATTTTATATACACTGTGATCCTTTTAAAGTACCTCTGATCTTTTTAAATAGTTTAGGGTTTCTTCGATTTCTTCTGTAGCAGATT
AAAGAAATCTGGGGGATTATTTAATCACATCTGAACCCCACAATAATTAGTATTTAGCATGTTCTCTTCCAAAATGCTTTCATTTATTCCAATC
CTTGCTAATCAATTGTAGCTAATTTTGTGGCCATTTGTTAGGAGAAAACAAGTGTTTCAGATGTGTTTCTGTGTCCCTTCCTTCTCTGCTAGAT
ATTAGCACCAATCATTTGTCATCAGGCTATACTTAGGACCCAAGCCCAGGAGGCATTTACTGTCCTGTCCAGTAGCAATACTGCAGACTTGGAG
TGGGAGAGTAGCCATTATGTCCAGAAACAAAATGGGGCTATGGATCCACATCCCAGAAGATGCCATTTGGTTTCCTCTTTATTGTCCTCATGCA
AGGTGCAAAACAAGCGAGTAGGTATTAAGAATCATTTCATCTTGTCTTCTCTTTGTGTGGACAGGAGTCTGAGGTCACATGCTCTAAGTCCCAC
AGTGATGTGGTAGCAGAAAGAAGAGGATGTGACACCAAGGATCCTGACTCCCCACTACATTATTCCTTCCAGTGAAAAATACCTCCCTGTGTAA
AAAGAGTTAGTTTTGTAGCTAAACTGTCTGTTTAAATACAAGCACCCCTTACTTGTATGCATATGGTAGGCAGCTCAGGTTTAAAAATGTCAGA
TCACCTCAGGCACTGCCTTTATCTCTCTTTGTCCCCAAATCCCTGAAATCTTTGGCACTGAAAGTATTAATGTCATCAAATTCTGAACCTATGA
AAACTTCATCTCTGTCTTTCTCTTGTCTGGCCTGTAACATGACAAGAACAGCTCACACCAAAGTTAGAAGATAATTTCATCAGTCATCACTTAT
CTCTTGACACTGCTTCTTTCTGTTAGCGTTAATATTTTGCTCCTCAGATCCTTTTTCTTTTTTTTTGGAGTCAGGGTCTCATTGACTGCTAAGC
TTGGGCGCGGTGGTGTGATTATAGCTCACTGCAGCCTCCCTGGGCCCAACGCGTCCTCCCACCTCAGCCTCTTGAATAGCTGGGACAATAGGCA
TGCACCACCATGCTTGGTTAATTAACTTTTTTGTTTTTAATAGAGATGGCGGTCTTGCTATGTTGCCCAGGGTGGTCTGGAACTTCTGGGCTCC
CACATTGGCATCCCATAGTGTTGGGGTTACAGGCATGAGCCACCATGGCCGGCCCTCTTTTAAAAAATACATAAATAATCTCAAAAGGAATACT
CTTCTAGATGCCAAGGAATTACGTAGCTTGTAGGATCTCACGAGCTACCTTTCTTAAAGGGTTACATTCTTATCCATTGTTTATCTTTAATAGT
AGATCTCTTGTATAAACAGAATATTATTAAAAGCAATTTGGGAGGGGGGCTTGATTTTAGAAAATAAGAGAATCGTGTGAAATGAAATTCCTCC
TAGTTTTCGACTCTGTGCATCTCTTGCCAATTTTAAACCTGGCATTCACACTGGAGTGACAAATTTGTGGAAAGCAAGGGAAGACAGAAAACTG
CTGTAAAATAGCAAGGGAAGTACTTTTCCAGCCATATTGGCAGTGCTATTCTGTACTGTCAGTGTGAGAAACATCTGAGAATAAGGATGCTTTT
GGCTTCTGCTTTCCTTTTACTTCCAAATCCTACCTGAATTGTAATTGAGAGAATGTAGGATTTTAGGAATATGCACTTGAATTACGCATCTAGT
TCTCAGATTAGGGGAGATTTTTCTGAAATTAATAAAATTTAAAAGATATGTTTGCTGCTTTCCTTGTGAGTGGTTTGCCAAAGCCCTCCCTTTG
TCTACACATCACTATATCATCCTGTCTGTGGGAAATGGACAACTAACATGGGTAGGCAGAGAAGATTGAACAGTTTGGAAATGAGGAAACTCAG
TACCACCACCATTCACATGCGAGTCAGTATGATATATTGAATAGCACACTAAGATAAAACTTAAGAGGCTGGGTTCTGTCATTTATAAGCTACG
TGTCGTGATCCAAGGTGCTTAACTTCTCTGGGTCTCATTTTCTGTTTTGGTCAAGGGTTGATAACAGTACCTGCTTTATCAATCTTTCAAGTTT
GTTTATGCACATGACAGCTCTTTGGGAACTGTAACATACTAAATAATTGAGATAATGATGGTGATGATGATAATTACCGGAAAGCCTAGTATAA
TATCATGCAGATAAATTGGAGCTCTTGAGTTTGGTAGTTGAATCTAATTCACTAGTTATTGATAGCATTGAATTTGGACCCCTGATATTTTCAA
TATTATATTTATCCCTATCATAGTACGCCATAATCATTGTTTTTAAGATTTCATGGTGGACACAGCATTTGAGCTAGCCATTCAAAGATGGTTA
AAATTTTGCTAACGGAAAGTAATGGTTAGAAAGAAAAACATAAGCAAAACATAGAGGCATTGTAGTATATGCTTTGTTCTGGGAGGAATCTAGT
TAGGTGGGTTACACAGGGTGGAATAGTAGAAAATGAGACCCAAAAAAGTGCCTTGAGGCAAAACTATTAAATGCCATTTCAAAAAGTTGGAACT
TGTTACTTACACTAACTAAAAATCGGGGGGAAATGTTGGGACTCGATTCTGAGCTGAGCAAGAAGCCACTGGAAATACTTCAGCAAAGTGGATA
TAAGACTTAGCTACAAATGACATGAAAAAGGGTCTAAGAAGGGTTCCTGTCTTGGATAACTGGGTGGCTGTTCACGCTATTAATAGAGACAAAG
AACTTAAGGGAGGAATGGGAGTTAGAATGCGTATCGGTTTTCCCATCTTTGGGCACATCCTTTTATCCTCTCTGGCCTGAAAACAAAGGATTAA
ATAATTTCTAGGAAATTACATTTACCTTGAAGTTTTGATACTTCTATAATATAAAACGCCGGAGTCCTTGTGATTTTGTTTAGAGATATATAAT
CGGCATTGTAGTGTGTAATGATGATGGACATTAAGGAACCCTTGTTTCTTAGTGCCTTTTGGAAACTGGCTCATTTCAGAAGCTGCGCTCTTTT
CCCAGAGGCAACTTTTGAAGTTACTTAAATATTTAATTTAACAAATATCATTTAAGATTTAACTGTCTACTATCCTTGAAAGGGTCATGAGTTT
CCCTCCTAAACCAAGACTTTGAAGCTCTCTGTGCTGTCTCACAGGAATCCCCATGATCCAACTTCATGTGACCTGCAGAGGTCTGCTGCCTTGA
GGAGTAGACAGGTAGCTGGGCTGTTACTGGCTAAGTCCTTTTCTCAGAATATGTGGAGGCTCCTTCTACCTTTAACAGTGCTTTTTTGCTTTGT
TTGGGAAGTAACAGAGTAGTCAAGTGCATGACCCTGGAGCCAGACTCCCTGAGTTTAAATCCTGTTTCTCTCTTTAGAGGTAGTTCCTTGGGTG
AGTTACTTAACTTGTCTGTCCTTCAGTTTCCACATCTGTGAAATGAAGATACCAGTAGTTACCTGAGTTTTGTTACCGATATCCGATGTGCTTA
GATGAGTGCTTGGCACTGGGTGCTACTCAAGTATCTGTTAAATACTTTTATGGGGGATAGAGGACAGATTTAAATGGTTTCCCATAGCCAACAT
TCTAGCTTTCCAGAATATCAGTATTTTGTTGTGGTAGCGGTGGTCTTTTTTCCTAAAGAAACAGTGCTTCGCCAGAAGGGAGACATTGTTCCCT
ACTGGCTCCCTACTTCCACCGTTTCTCCCACTCTTCTATATAAGTGCCTTAGAGAAACTCTGGTCACCCTTTTCTGTGGTCTGCTTAGCTCCAA
CAAGAATCATAGACCAAAGGTTTTGCTGTCATCACTTTAAAAATAGTGTAAGTTATTTTTTTCTTTCCTTTTTTTAAATTTTTTTTTTTTTGAG
ATGGAGCCTCAGTCTATCGCCAGGCTGGAGTGCAGTGGCGTGATCTCGGCTCACTGCAGCCTCTGTCTCCCTGGTTCAAGTGATTCTCCTGCCT
CAGCCTCCCCACTAGCTGCGATTACAGGCATGTGCCACCACGCCCAGCTAATTTTTGTATTTTTAGTAGAAATGGGGTTTCACTGTGTTGCCCA
GGCTGGTCTCGAACTCCTGACCTCAGGTGATCTGCCCGCCTCCGCCTCCTGAAGTGCTAGGATTACAGGCATGAGCCACCACGCCCAGCCAGAT
TATTTTTTCTTTAGTGGAATTCAGCCAATTGAAGCATAACACATGGCATGAACACTCTGGAAAGGTGGCTTTGGATCTTTCCATTTGTTCTCTT
TAGCGCTGACTTTTGCTTTCTAGTCCCTGCTAGTGTGTTGGAATCTTACCCCATACAGATGCAAACTATGAACCAAGAAGTCAGCATCTATGCC
TAGCCAATAATTGGTTGTTTTCTCGTTCAGAGTTCCTCATGCCCATGGGGTAAATTCATATTTCATCCATAAGTGTGACCATTGCAGAAGTTCT
AGGACTGCTACTGTCTGTATTTTTGTCCTTCAGCATATCCCAGACTAAACTTACTCTTAAAATATGTTTGTCCAACCTTTGGTTTCCTTCCTAT
GGTTGATATTAAGCCTTTCTTCTACTCTTAGAAGACAAAATGGGGAGACAAAAAAGGCAACCCTGAAGGTTTTTATCCTTTCAGGGTATCTAGC
ATTTTTCTAATTTAGCCCTACTGAAGTTTCTTCAGTAATTAATCATCTGTTTTTCAGCCTCTAACCTGGCTAATATTATAGCCTACAATTGTAC
TTTTAAAGCCCTTTTGTGACTTTCACATATTGTGTCTATTGTGTATGGAAAAGTAGCAGGATCAGTATGAAGATAATACAGTATCTCTTAAAAC
AGGCAGCCAGCAAATGAACTTTCTGCATTGGTCAGAATTTCCATCATTTCACTGTTAATGAGGAAAGTACAGTTCTTTAGCTACCATGAAAGTC
AAACATATCCTAAGCCTTTTGGAAAAAGACATACATGTTAGAAAATCTCAAATGGATGAGTCAGCCTGACTGACCCCACATTGACTCCATTTTA
TATGCTGGCCAAATCCTGTTTCTGACTTTCCTGGCACAGCCCTGGACATGCTTCTGTATCATAGGACTTGTTCCCCAGCGCCTTTGCTACTTCC
TTCAGGCACATTCCTAGGAAAGATTGGCAGTCGGGTTTGCTCTTTGCCAGCACTCCTGCCGGTTTGGGGGTTATGGATGCCAGGTTGGGCTCCA
GGCACTCTGCTCTCCATTTGTGGAGGCAGGCACCGTAACAGCACCTGACAAGTAGGGATGATCACATTGTATTCAGAAGCCTGGTGGAGCTCTA
TAAACCCAAATTTCTAACAGTCTCCAATGTAATGCCCTGTAATAGAAGCTGTCCTTAACCCTCAATCATCTGTATTCAGCTAGTATAAAAATGC
AAATCTGCTCTTATGCCTAAACAGTTAGGAGTAGAGGAGACTTGGCTTCCTTTGGAAAGTCAGGATGATAGCTTCATCCCATTCTGGTTTTTGT
GATTAATTGTGACCTACAAAGGGGTTTACTACTCTAGCAGGAACTTTGAATTTCCTATATACATCTCTTTCTGGTTAGCAGGCAGAGGAAATAT
CGTTGACTTTTGGCTTCCTGGCAAATGTCTCATTTGCCTTGTCATTTGTTCATTCCTrTTCCCTGATCTCTGTTTGGTGTGATAATGTACAGCA
AAGCTGAAAACCGCAGGGCTACATGTACACTTGTAGGTACCTATGTTGTGATTGCCAAAAGGCTCAGAAAGCCAGTTTTCTAGTGAAAATGGCT
AACATTCTAAGAAATGCTTTCACTGAGAAAGAGAACGGCTCAGCGGAAGGTGGAACTTAAAGGAAGATGGAGTGTTCCTGAATTCAGATTCGTG
AATTAGCCATTTATAGTTTCATGGGAAGACAATGAAAAAAAAAAAAAAGAAAGAAAGAAAAGAAAAGAAAAGAAACAAGCAACCCCAAAGGTTT
TTATCCTTTCAGGTATCCAAACATCTTTATTCTAGAAAGTTTCTCCTCGATGGTAAAATAAAGCTAGGACCTTATAGCAAGTCTCAGACTCACC
ATGGCTGTTTCTTAACACCAGTATACCTTAAGCAGTAGGTATCAATTTAGTGCAAATATAAGGGTATTGATATGTAGTGGAGGCTGCTCTGAAC
TTGGAGGTGGAAATACTAAGTTTAAATCCCAGCTTTTCTTTTAACATTGCAACTTTGAGAAGTCTTTTAACCATTGTCTCCTGTTTGTATCAGC
ATGAGGGGATAGATAATAAAACCTGCCTCACAGGAGTGTTCTGAGAATTGAGTGAAATAATGTATGTGAAAATACCAAGTACAATCCTTGCTTA
GAAAGTAGAATTTTGTTTTGCCATCCCCTCCCCCACTATAGTTGGCAGGGCAGGGGGAGGCAGTATATTCATCCATCAATATTATAGATGCAAT
TGTTCCCAGATGTTTTGCTTTTAGGCAAAACCTTGTATAGATGACTGTATACCCTCTTACAGTTGATGCTTAATAAAACAAAACTACCATGGTT
TTATAATGGAATGGTAAGCTCAAACATTTTGCTGGTAGTATAAAAACCAAGTGCCTGTGATTACCTATACATTAACTCCTGAAGAATCAATTAT
AATCTCAGCAACCTTGCTCTTTGACCATTCATTCTAAAGCTTGGCGAAGTTAATGAGAACAGATAATACGAGTGCTAATGCAATTCGAAGACTT
TTTTTTTTTTAAACAAAGTAAGGAAGGGATACATTTTTAAATAAAACATTGCATTTTGGTAAAAAACACAGATTATGAGAACACATTGAGTTAG
AGGAACATCATATTGATTCCAAGAACAAAAAGCATGGTGGAGAAATTTTGTAACCATGCAAATCCAGTGTGCTCTGTTCTCATGAGTTAAAAAG
AATTTTCCTCCTATAGAAACTTGTTATTTAAATGTGTGTCACATGTAAATAACAGTCTAATGAGAGCAATATAGAGGGAGAAATATGTAAGCAA
GGGAAAGTGGGCATTGTTTTGTTTTTTTTTTTCTCCATCTATTTCAATACTTGGGTCTTTCCTAACCTATAGTGTGTCCTGTTTCTCCCTGGCT
AGATTTCTTAAAGTTTCTGCCTATTCATCACTCAAGTACCTATCTCAAGTTGTAGCTACTTTTGTGTCCAGGGTGTAATAGCAGATGTTCTCCT
GATCTAGATTTTGTTGCTCTGCCTAACTGCATGCCATCATTTGTTTCTCCCTTTGTGATCTTCTACTGTACTCAAGTGTCCCTTTAGAACAAAC
TTCATGGGATGGACATAGCCTAATGAGCTCATTGCTGATGAAGTCTTTTTTTCCTGTGCATTGGTTAGTGCAGTAAGCAAGGCTCCTTAACTGC
CTGTCCCAGTGTACTGAAAAATAATAGTGTTCTCTCACCTTAAACTACAACTTCTGGTTTTTGCTTATTGCTTCTTTAGTCTTTGACCACATGC
ATGTTTTTACATAGTTGCAGTTATTTTATTATATTCCTTTTTCTGTCATAAACACTTTCCAAATGTGTTTGTGGTACTCAAATTATAGCTTAGC
AGTTAAATAATTTGATATATTACAGTTTCCCCTCTTGTTAGGTGTATCCAATTTTGGGGTTTGTTTGTTCGTTTTTCTTTTTTTCTGTTGTTGT
TGTTGTTGTTTAAGGGAAGCACCAAAATAAACCTCTTTTTAAAATTAATTAATTAATTTTTTTCAGGCAGAGTCTCGCTCTTTCGCCCAGGCTG
GAGTGCAGTGGCACTATCTCGGCTCACTGCAAGCTCCGCCTCCTGGGTTCATGCCATTCTCCTCCCTCAGCCTCCCGAGTAGCTGGGACTACAG
GCCCCTGCCACCACGCCCCGCTAATTTTTTGTATTTTTAGTAGAAACGGCGTTTCACTGTGTTAGCCGGCATGGTCTCAGTCTCCTGACCTCGT
GATCCGCCTCCCTCGGCCTTCCAAAGTGCTGGGATTACAGCCGTCAGCCACCGCGCCCAGCCCAAAATAAACATCTTTATATGTAATGTTATTT
CCCTAAGATAAATTTCCAGGAGCTTGATCCCAGGGTAAGAAGCTATGAACATTTTCATGGCTCTTGCCTTATAATGTCATATTTTGCTTTTTAA
CCTTTAACAAAGAACATTCCTATGCCCTTGCTTTCAGATGGGTCTTTTCTTATTGTGAGGAAAGGGCTAACGTAAGTCCCATCTAGAAGTGACT
AACTTGCCTAGTTAAAACTAGAAGGAAATATCCACTGTATTGGCCTCTTTACTCCATAAACCCTGGCTTGGCTACATGTGTGGTATGTTTCAAG
TCTGGCACAAAGCCACCAAACACATTTTTGGTGAGTTGGTTTTAACCTGCTCACATCAGATATGAATGCTGAAGTTGGAGCTTTCATTATTGTT
TTTGTGCTGGTAGAATGTTGCAGAAATACACAGGGCTGGCTGTGTATTCTTAGTCACTTTAAAAAAAATGTCCTCGTGTGTCTAGAATGATGGT
TTCTCTTCTTTGCAAAAATAAAAACATCAAACAAGATAAAGTCCGATGTAGCCAAGACCCTCTACTTGAACCTTCTGTCGGTACTGTTTGTTTA
AGTACTGTAAGTTTCCACCGTTCATGTTATCTTAGGATATCTTGTGAAACGAATCCTTGTGGTCCCTACTCTGGGCTAACGCTTTGTGACTTAA
CTTTTTACAGTGAACATAAAGATTCTGAAAAGAAACACAAAGAGAAGGAGAAGACCAAACACAAAGATGGAAGCTCAGAAAAGCATAAAGACAA
ACATAAAGACAGAGACAAGGAAAAACGAAAAGAGGAAAAGGTACAGAGTTTTCTAGTAGGGGAGGAAGGAACGTGGATGCACTTTTGTTTACCT
TTGAAAGCAGATATCTTAATTGCATAGTAAGATCCCTTGGTCTTGAAGTGAAAGGTTTCTTGTCAGGAGAATTTAGAGCAAACTGTAGTCTTAA
ACTATAAAAACTAAGCCTTTTACCACCATATGTTTCACAGTGGCAGATCTGTCCATATCATTACCTTTTTTACTTTATATTGCCAATGAATACA
TTGAGAATGCATTAGGGAAAATTAATTCTTTTTGGGAGAAGGCCTAATGCCTTGATGGTCTTCTGTTGATTAAAGGTTCTTCCCGAGTGAAAAT
TTAGTGATGCTGGTGTCTTTTGTGGCATTTGGTTCCAGCATAACCAGTCCAAGCCTTCTAAAAATGACTAGGCTCACCCACCTGAGAATGAATA
GTACTCTGTATAGTTTTAAAAAGAGATTGTTTTTATTTTAGCTTTGAAAAAGTAATTTTTGTACTTGGTAAATTCAGATACTATGAAATAAGGA
ATATGGTGAAGAAGACATCTCTTCACCCCTGACTCACTGTCTCCCATCCTTCTCAGAGGCAACCGCTATACTGATTATGTTTTGTATTCTTCCA
TAGATACTCTGTATATCTGTATGTGTGTGTGTATCCCTTTGTGTGTATATATGAAGATTTTTTTGAAAGATAAATTTTCTGACGAATCTAGAAA
GCAGGAAGTCAAAGGGGAGGGGGTCATGAGTATACTTTGCCTAAGTAATAGAAGACAGGGCATAGACTACTTTTTTGTTTGTTTGTTTTTTAAA
TAAAGATGGGGGTCTCACTGTGTTGGCGGGGCTAGTCTCGAACTCCTGGCCTCAAGCAGTCCTCCAACCTCATCTTCCCAAAGTGTTGGGATTA
CAGGCATGAGCCATCACATCTGGCCCAGTTTATATGTTTTGTTTGTTTGGTTTTTGCTTTTGTTTCCTCTCTGCACTCCCAATGGCAAATTCTT
GAGTAGTGCAATGTATTTATTGGGGAAAAGAGACCTGCCACCACTTAAAAGTAAAAAGCTGCCATTTTTAAGGGAAACCTTTCTCAGTGAGTTG
GACAGTTTCACCCAGAAGAATTGTTAAAAACGAGTTTTGGACACATTTCAGGACCTTTAGTTCATCTGTTCAGTTTTGGTAACATAAGAGCCAG
CTTGTCATCATGATGTCCCAGGAACTGATGCTACATATTCTCAAAAAGAGGTTTCTTCAAATTTAGTCCTTTCAAGGTACTAGTTACTGTTGTT
TTACTTTTCAGGTTCGAGCCTCTGGGGATGCAAAAATAAAGAAGGAGAAGGAAAATGGCTTCTCTAGGTAAGACTTTGCTGCTGCGTGGGCTTC
CCTTTCTCTGGGTGGACAGCAGGCCAGCTGCCTGTGTTGTAGTGTTGTCTGAGTTAACCCAAAACCCACTGACCTGGCCATCTTGATGGTTCCC
AGAAGCTCAGCAGTGAGAAGACTCGGTCTAAGCCAACCTGAGCCTTTCTACAGTGCCATATAGTGAGACTGACCAGCACATTTTCCAGAAGGTT
ATTTCGAATAGAGAAGGTTGACAGGAGAGATTTTTAAAAATTGAGATTTGACTTGCATATCACAAAATTTGTATTTTAATGGTATACAGTGTAC
CATTTTTTTTTTTTTTTTTTTTTAGTATATTCACAAGGTATTGCAGCCTCTTCACTAATTCCAGAACATTTTCATTTCTCTAAAAAGAAACTTC
ATATTCAGTAGCGATTATGGAGTTTTCTGAAAAATCACTTAGTCTGATTTCATCATTAGTAATATTGGGAGGCTTATATATAAAAGATCATTTG
ACCTACATGTTAGGGATGAGTGTAGATTTGGGTAGAATTATGTTATCTCTTTTCTAGAAGGAGAGGAAAATGCGCAAAATCTAGAGGTTACTTC
CTATACTGTTTTTACCTTTATAATTTTGTTTTCCTCAGCCTCAATTTCCTAAAATAAGCCCAGAGGTCCCCTCTAGTTTCGAAACAACTAGATG
TTTATTGTTACATATTCTCACTTCACTCAATGACAGAAAGGATTTATTAGAAATGGTTGTAAGTGTTTGCATTATTGACTGGGAGAAAACACTT
TGAGAGTTTTGCTTCCTTGAATCTTTGAACTTTCAGTTTCATTTGTTAAAATTTTTGACAATTGTGCCTGGTACTCTGGGCTCCTTTAGACAAG
GAGAAAAGCAGGTGCCCTCTTCAGGCATGTTTCATAATTGTTCTAGGAATGCAGAATAATACAATTCAGGAAGCATTAGAGCTGCAAGCTGCCC
TAGGAATTTTGATTTCTGAGATAAAGTGACTGAGGAGGCACTGCTGGGCTAATCTCTGCCAAGAGACTGCCTTGCATTTTTATTAAACTGATAT
GAATAAATTTTTTGGACTTGGTGCATAGTCATGAATTCATTTTCAGACTTTTTGGGTTAGAAGATTAGGAACACAGCCTTCTTTGAGGCAGTGG
TCTCTATGTTCACTTTGCTGGACTCCTTGCTGGCTTGTCTATATGTCTTTGTACAGAATCCAGATGCTTCACTGGAATACAGTTTTCAGGTAAA
TTGTTTTAGGACTTCTTCCTTCAGACCTAAGTAACCCTTCCCACAGTTTTTGTCTTCAGTGTGACAAACTTAGGATCTAGGATTTACTTAACTA
GTTGGGTAAATGATAGGCTTTCTTTCCTTCTGGACCCTAGGGTCTGTGTCAGTATTCAGGCAGGAGGGGGAACAGCTGTAGAAGCTGGCATTTG
GTTAGGGTGCTAAAATAAGTTCTTTAAACATTCAAATTCTATCACAACCAAGGGGACCCACTATGCTTTTTTTCCTCCGAGGTACTCTTGAAAC
CCTGTGAATATACTTTGCAGTGGGGTCGGGATTCTTAATCCTTGGATCCCTCCACAGGGGGCAGCTGCATGTAAGGAGGCCATTTCTTGTACTA
AGTTAAGGCAGTGAGGTTCTGAAGAGATGTGCCAGGCCAGGGAGCATCCCATGGCTGCAGTGATTGCTGGGCTCTATGGTGTTTTTTACTTGTT
CCTTCTCTGTTTTGGTTCAAGATGTTTGTCTCAAAGGATTGTCTTTTTATATTTATATTTTGAAGGTAAGATCTTAACAAATAGTAGTGAGACA
CTCTTCTGTATATAAAGTAAACCTAAGTTAGGACGTTGAACAATGAATAGATTTGGCATAGGATACCTTCTGTTTATTCTGGTTTAACACGTGC
CCTACTTTTGTTGTAATGTTGTCAGCTTGGTTATTTTTCCATGTAGCTAGATATACATGTACGTTATTGCCACATTTTTAGAACTTTAAGGTAC
TAAGTTGGGGAAATCAATGTTTATTCCTTTCCTTGTAAAAGCTGTACTTTAGCTTTTAGAGTACTGGAACAATCAGTTCAGATTGTGGGGTCAA
ATATATGGTAGGTTTCCATGTAAGGCAAGAAAACACGTTAGGTTTATTTCTGGAGTGGTTCGTGCCATGCTGGCAAAGATAGCTAAGAAATAGT
GAGAAAGAATTCTCACTTAGAATAAAGAGTCTTGATCCCAAATCCTGATATTGCCCATTCACCTGTACAATGACGACAAATACTATCTACTTCA
CAGGATTGAAGTGAGAAAAAGTGCTCACAGAACATCTTCATGATATGTACGTGGTTATCCTTATTTCTGTTAGCTTCTTTTCAACGAAATGACA
TATTCCTTCTTTGTATTAATAGAATACAGATGTTCTAGCACTCTGACCAGCAATTTTTTTTCTCTTTAGTCCACCACAAATTAAAGATGAACCT
GAACATGATGGCTATTTTCTTCCTCCTAAAGAGGATATAAAGCCATTAAAGAGACCTCCAGATGAGGATGAGTGACTATTTTCTTAAAACTTTG
ACTTTTGAAAACAAAAAGGAGGAGTTTAAAGAATAAATGTGATGTGTTTCTTTATAATACATATAGAAACTGCATTAATTGGCTTTTACTCATT
TGGAATTTGTGATTAATGGACTGATTTCTCTGAACTATGAGAAGTAGAGTTTGCTAAACAACTAGTATACCCAAGGCTTACATGAAGATTATAT
AAATTTTTATTACTGAAAAGGCAATAGATATTTATTGTAGAAATTTCAAATAATACAGAATTCATGAAGAATAAATTATTTAAGTTACTTAAAA
TTTCTTATTGCCTACCACAGATAACTTAAAGCAGTTTCTCTTAGACCTATTCCTGTCAGACATTCTAAGTGAAGTTTCACTGTGTAAAAATAAT
AAAACTGCTCATAAAACGGTCGAGGCTTACATGTATCTCTTTCCCCAGATTCAATGAGATTAAATTATATTCATTATTTTCACTCTTATATAGC
TTACTATGCTATTGTTATAGTAGAAATTGGATGGAAAGGGAGAAGGGAAATACTTAGTGAACTCATTTGTATTCATTCAATATAGAATTTCTTT
CCTCCACATTCCAGTGAGTTACTGCTATGTGGTAGCTAGTGTGCTGTGTGTTTGCTTCCCAAACTTTCATCTTTTATTTTTATTTATTTTATTT
TATTTTTTTTCCCAGACTTTCATCTGATAATGATATTTATTTCTGCATATATCCCCCCACCCTATTTTGCTTCCACGCCGGTGTAAGTGGTTTG
TTTTGTTCTATATAGGGCTATAACTATTACTCTTTGGTCTGACCTTCATTATTTTCTCTATTTCTGTTAGTCTTGACCGTAGAATGCCCAGCCA
AGAATAACAGTGATTACTCTTGCCATGGAGGCATCCAAGAGTTTGTAGGTCTCCTATGAGTGAGAACTCCTGAATCATAATTATGTTAACTGTG
TATTCATGTTCCCCTTTCTAGTGCTGATTATAAACCTAAGAAAATTAAAACAGAAGATACCAAGAAGGAGAAGAAAAGAAAACTAGAAGAAGAA
GAGGTTACTAAAGAGACTTAGGTCCTTTGGGGCTTGAGTTTCGAAGTGGGAGTTTTCCAGTAAGCAACTTCTTAAGACAAATGAGTTTTAGATA
TTTATTGGCTTGTTATAACATTAGGGAAAACAAATTTAATAAGTGGTGGCTGTGGCACTGGTAGTTTTAAGATCTCTGTGAATCCCCACAGTGT
CTTAAGTTAACCACAACCACCAGTGATTCTGCTCTCAGCCTTCCTGGGTGTCTGTCTACCCACAGCTAGGCTGCCAACACATGTTCTTCCAAAA
ACTATGTCATCTTCATATATACAGAGGCAGTGTTAACAAGAAACCACATTTTGGCTTATAACCCTGAAGAAATTTCCCTAAGAATTTTTTGCTG
ATCATTCACTTTAGGTAACCATTCCTATCTAAAGTTCTTTAATGGGTACCCCTTTGACATATAATTGACACCCAAACTCTTTACTGCAGCTCCT
CCCAATCTGGCCCCTGCATCTTGTGTCTCTCTTCATCTTGTGCCTCTTCTCTAGCTTACTTGTACCAGTCACACTAGCCTCCTTTGTTTCTTTT
CCACACCAGATTCTTTCAAACTTCCGAGCTTTACGTAGCACCTGCTGTTCCTCTATCTGGAACACACATGCTCCCTTCTTTATCCTTTTGGCCT
ATAACCTTCCTCAAAAAACTCAGTTCCGTTTAAATGCTTTCCTAAATGTTCTTGTTCATGCTTGTGGTAGATTGAAAATAGAATCATATGCTTC
CTCCTTTTGACACTTTTGAGTTCTTGTACCAAAAAATTGTTAGCAGGTAATTCAAGAAAAGATGTCAGAAATTCACAGGGGTCTTAATGATGCC
CTCCCCTAAGACAAGAAGGGGTTGGGTGAGTTCTTAGCTCTTATGCTACATTTGAGGTTAGCCACTGTCATGGGCAAAATGGGATCACAAATGT
AAAGTGGACAGTTTGAAGTACCAGCAGTAAAGAGAATGATAGTGAAGGGGCAGGGAGTCTTAAGAGGATAATCTATCATAACAGAATGATGTTA
ATAATCACAATTAAATGTAATAGGTGCTTACCATGTGCCAGGCACTCTTCTATATATGTATCATTTCATTTAATCCTTACAACCTGAATAGGTT
GTTACATCCCTATTATCATCTCCATTTTGCAGATCCACAAATACAGTATAGAAATATTTAATCATTTGCCTGAGGTCACATACCTACTAACGTG
GAAAACCCAGCATTTGATCCCATGCAGTCTGACTTCAGTCCCAGCACACCAGCTACTATATTATCCTACCTCCCCTGGGACTTTGCTGATGCTC
TAATTCTCCAAGGAGGGAGAAGAAAGGTCTTTAATGAAATCATATCAAGACCATTAGTGAGTGAGTTGAGCCATAGAGAGATGATCAGGTCCTG
AATTTAGAAGCTTGCCGTTTTTTTCACAACTAGTCTATTCTTGGCCGAAGTTCAAATTAATAAGCACTAATTCAAGGAATTGAGAGTGAAATGT
TAAGTAGTGATTATCGTAGCCAATCACTCCACTTGCATATACCAAATCAGTACAGACTAGTCAGTAAAGGAATATAAAACTTGAGTTCTCCAAG
TGTTTTTTTTTTTATGTTTATATTGCATTTATGCAAAAATAATACTCTGAGTTTGTTAAACCATTGGGAAAAAAGAACAGTTGACTTCATTTGT
TATTATTCTATTTAGCAGCCCTACCACATAGGGGGAAAAATGCTGGCTGAAAGACTGAATTAATACAATCTTACTGTCGCACGGACCCCAGATG
GCAGAATCTGTCATTAAACCTCACTCGCCAACTATAGTTTTTTTGTTTTTTCTTTTTTTCTCTTGTTTTTGAGAATTGTTCTGAGGTCTCTTAG
TGACTGACACCCCAATTTGAGGCTTGAAAACATACAATGTAGCCAGTAGTTTCCTGATGGGACCAGCTGGATGCCTGCTCAGAAGCTGTTTAGA
GTATCTTCACTGAAAAACATTTCCCTTTCCTGTGGTGTGTTTCACCCTAAATAGTAAACATTATCTGAATTAATGTCATCCTGTAGTACCTTCT
CCCAAACATACCCACATATTTATATTCTCCGTGCCTGTTTTAAAATATGGATGGTACTGTAAGCTTTCCTCTTTTTTCTATTCCTATCTTGATT
GCCATCCTATTATGAGGATCTAAAATGTTTATTCCAGTTATTCCCATAAGTCACATTCTGTTATTTTTCTCATAAAGTTAGGCTCTTATCATAG
TTAATAACAGCTATATGACCTTGATCAGTGACAAAATATGTTTCAAAGTCCTTGATTGTAAACTGGAAGTGGTTAAATGAACCCATCCATACAC
TTTCCAGCTCTGCCACTCTTTAATTCATTTTAAGATGGTTCAAAACCACCTTTCATTGCTATATAAAACTTGTTGAATAAAGACTGAATAAGTA
GTTAAATTATGTTGAATAGGCAGGACAAAGGAAAATATTCTTCAAAAGAGTTCAGATTCTGAGTCCGATCTTCACTTGTTGAATATTACATTTT
TGTTTGGGTTTTGTGTGTATGTTTTGAGATTCATCTCCTGTATTGGTGAATTGTGCCTGTTTAGGTTTGATGCCCGTTTATTGTGTCACTGTGA
TCAGCTCATTTACATTCAGAGTTCAAAGAAGAGCCATAAAGAAATACCACCATAGGAATTGCTGTCATTTTAGAACCTATCAAATACTTGCTTA
CTCCATCATGACTTTGTGTATACAAGCAGCAGCGATACTACTATACTCCACTAAATCAACTGGGAATTTTCTTTCATCCTGAATACTAAATATC
TAATGCAAAGCCCCGTCTAGCCCTTATTTGATTGTACTGGAAGAGGTTTAAAGATGAGTAGGATTAAACATAAGTGAGGAAAATACACTCGGAT
TACTCTCTAGAGCAGTACTTTTCCAAACTCCTTTCAGGTTAAAAAAAAAAAAGTTTCCCAGTCTCTTCCCCTGCAGAGTGTTTTTTAACTACTT
TCTCAAGTAGTTGTTTGTCAGGAAATTTTAGCAAATACTGCTGTAGAACAGAGCACCAGTACATTGTTATTCATCTGTCAAATGAGACGAGTAC
TAAGATCTTCTTTAGTTCTAATACTCTATGATTACAATTTTTCTTCCTCATGGCTCCCAAAAACTGCCTCCAGAAGTGTGATCAGTCTTTATTT
AAGTGCTTTCTCACCATGTTTCTTTGTAGGATGGTAAATTGAAAAAACCCAAGAATAAAGATAAAGATAAAAAAGTTCCTGAGCCAGATAACAA
GAAAAAGAAGCCGAACAAAGAAGAGGAACAGAAGTGGAAATGGTAACATCTCAGCACTGGGTTAAAATGCCACCTTTTTTATTGCATATCCTTA
CGAAGCAAGAGTATTGAGTTATTAAAATTAATATCCTGATATTTGGGGGGAAATACTTTAAACAATTTCTCTAGAGTAACTTTCTGAAGTAAGT
TTTCCAACTTCTAGTGAAATCATTAACTGGTTCTAAATCTATTGGATCTCAAACTGGTTTCCACCACTGTGAACAGAATGGACTGTAGAAGCAG
ACCTATGTATAGGTGGGAATTTAGCATACGAAGAGAGTACAGATGCATTTAAAACCATACGGAAAGGACAGATTATTATGTAAGTCGTGTTGGG
AAAACTGCCTGTTCAACAACAGAAGAAACCTCAACAAGAAAACGTCGGTGAATATTTTTATAATTGTGCAGTAGTGAAGATCCTTTGAAGCAAG
ACATAACCAGAGCCATAAGGAAAGGTGTTTTACATTTGATACATCAAAGTTCAATTTATGGCAGGTGGACACCTTCATGCCATTTAAAAAAAAA
AAAAAAACTCAAGGCAAAATATTTGCAATACAAAGGGCTGATATTCCTAATATGAAAAAAAGCTCTTTTAAAACAAATATGAAAGGAATGATCT
AATCAGAATGTGAGGAACAGTCAGTTTAGTAAAATAGTTAATAAGCATCTAAAAATGTGATCAACTTCATAAGTAATCAAAGATGCAAAAAAAA
TGATAAATCTTTTTGTTTAATGCCTCTCAGATTGGGAAAGATTAATAAGTGAAAATATCTAGAGTTGCAAATGTAAGAAAACAGGCATTCGCCT
TCCCTATAGCAGTGAAAATTCAGTCTTTATGAAGGACAGTTTAGTCATAGCTGGTTGCTCTTGAGAAGTGTATTTAGGTGGAGAACTTTTCTAC
TTTTGCTGTTTAGAATCTTGAAATAAACATGTTTTTAAAAAATACTTATTTCTGTTAACATGTTCTATTTAACTACTATACCAGCAGTACATGA
TTTTGTGTTTATGATGAAAAAACTCAAATGATTGACAGATTTAATAGTTAGAATAAAAATCAAAGTCACCTTTACCTGTCTTCTCCCATTCTCA
TTTTCCTTTCCAGCTTTCCAGCTTTGAGGTAATCACTGTTCATAATTTGATGTATAGCCTTCCAAACCTGTTTATGTCCCTTTATGGCTTTTGT
AATTTAGAACTTTGATGTCCACAGCACCTCTGAGGAAAGGGAGAAAGAGCACTGACCAGGAACGTGGCAGAGGGAAGGAGAGCCACAGTGGCAG
AAATGTGGCCACACGGTGACAGCTATGTGATTCGCATGTTGCTATGGCATCATGGGATTAAATGGGAAGTATAGAAAAGGAATATCACAGAGCA
GAGCAACCCTGCCTCTTTCTAGAGGTCAGAGTCAGAAAGTAAAAGGCCAGGCACAGTGGCTCGTATCTGTAATCCCAGCACTTTGGGAGGCCGA
GGCTGGTGGATCACCTGAGGTCAGGAGTTCGAGACCAACCTGGCCAACATGGCGAAACCTCATCTCTGCTAAAATTACAAAAATTAGCCGGGCG
TGGTGGTGCGCACCTGTAGTCCCAGCTACTTGGGAAGTTGAGGCAGGAGAATTGCTTGAACCTGGGAGGCGGAGGTTGCAGTGAGCTGAGATCA
CGCCACCGCACTCCAGCCTGGGCGACAGAGCGAGACTCTGTCTCAAAAAAAAAAAAAAGAAAAAAAACCCCACATGGGTTTATTTTCCTCATTT
TTATTTCACTTTTCTTAAGAACTAATACATTTTCAGTCATCTTGACCTTTAAGAAAATGATTTTTAGAAATAAAGTGATTGTCTAAATTAGAGG
TTATTATTGGGAGTTAAGAGAATGGTAGGTTAGTAATTCATCAGTCAGGGTTCTGTGGTAGTTACTCAATAAATGCTTGTTAAATGAACAAATA
CATTTCCTTATCAGGACCTCCCTGCTGTCTCCCCTTTACTTCGGAGCTTTGGCTCTTTTTGGCATCCAGGGAAGTGCGCTGGGTCCAGCCTTGT
ATGTGTAGAATTAGGTGCCCTTCAGGGGTTTAGGAGCCTAATTTTGCTTTTGTCGTTGAAATCCTTATCAGTGTTGCCACACATTCACTCTGAC
AGACCATGACTTTTGGTTTTTATTCTTATTTTGGGTTAAAGGGGAACTGGCTAGTCTTTAACTAAAAGTTGTTTTTTGAGGAATGAGCAGACTT
TCCTACAGCTTCTAACGCATGCATTTCTCAGGTGTGATGGTTAAACCTTGCACATTCTTTTCAAGCACAGTGCTTGAATGGCACACCGAGTTTT
TGGTGTTGAAAATATGAATGAAGAGCCTCTTAACTGATAGCTAGAGGCTGTGTATCATACTCTGAAGTCAGTTATCCAGTCCTCAGTGACTGCC
ACTGATGACCTAGTCGGGGAGTTAAATGGCAGATACCCTTATTTTTACTATTCCTTATGCTATCCTACTAAAAGAAGTACACACTTTACAAGCT
TGACAAATTTGCAGATCTTTTTCTTCCCTCGTAAGTCCGCAGACCTGTTTGTCTTTACCAGAAAAAACCACTTACTCTGATCTTTATCTCCCTC
CTTTGGTAATTGTTCAGTTGGAAAAGGTCCAGGTCCCTCAGGGTTTCTTTGCTATATCACGTTATCTCCATAGAAACATTGTTATAAGTGGTGG
CAGTGTGCTTGGGTTCATACATGAAATCCTCTTTATCCCAGATGTCCTGATTTTTAACTGTTGGCCATTGTAGCTGAACAGGGTTTCAGCACTG
TTGGGTTAATTAAGTACACTTTTGTGTCAGGGCTGACCTGGCAATATAAGTTGCCAGTTAATTCTCAACACATGATTCTTCATAAAGAATTACC
TGGCGAGTTTATTGCAGGTGTTAGGTGTGACTTTAAATGGAGTTTTCAATCTCTTAGCAAAGGTAAAATGCTGTGATGAGTCAGGACTATAATG
TTTTAAGGAATTAGTGTTGCAAGACCAAGACTCATAAATATATGGTCAATGATAGTGGTAAGAAATTTATGGAATATGGATTATTGACATTTTG
CTCAAACCAGAAAAACTTCCCTTAATTTCAAGGATTAGCATTCAGAAGCTACAGCATCTGACCTTTATTTCTAAATTTAAAAAAATTAAATATG
AGTTTTATAGCATACTGTGAAGTGCTTGTTAACTGCAAGGGAACTTGATATCAAACAGCACATGGAGTTTACTTTCCTGTCTTTATATTTCTAG
CACTTTTCCACCTTTCCCCCTTCAAGCAAATTTTATTATTAAATTGGTACTTGATTATTCAAGTCAAGCTGCCAAAGGGAAGCCATTTCGATGT
CTAAGTACATGTTTTTGTTTGTTTATTTGTTTTTAAATTGCGGGGGGCGGATATATGCACTCATGGGGAGGCTAAAAGGAGAGAACTGTTTGCT
TTTATTTGTATAATAATAGCTGTCAGCCCAGTCACATGAGCTTACTGAAACAGATATTCTGTCTTTCTGTTTAAACTGAAGTTTAGTGTTGTGA
TTTGCTGAATCTCTTAATTTGCTTAAATGGTAAAGAAGTTGCTGTGGGTCTTCAGGATACACATGTTGAAATAGGAGTCGTAAAAACAGTTTGG
AGTCTTGATTTAAAAACACTGTAGGGCCGGGCGTGGTGGCTCACGCCTGTAATCCCAGCACTTTGGAAGGCCGAAGCGGGCAGATCACGAGGTC
AGTAGATCGAGACCATCCTGGCTAACATGGTGAAACCCTGTCTCTGCTAAAAATACAAAAACAAAATTAGCTGGGCGTGGTGGCGGGCACCTGT
CGTCCCCAGGTACTCAGGAGGCTGAGGCGCGAGAATGGTGTGAACCCTGGAGGTGGAGCTTGCAGTGAGCGGAGATCACGCGACTGCACTCCAG
CCTGGGCGACAGAGCGAGACTCTGTCTCAAAAAATAAATAAATAAATAAAAACACTGTAGTCGATGAGAAGAATGGTGCCAAAGTCCCAGGATA
AGGGAAAGGGTGGCGTTCGGAATCTCCTCACGGTTACTCTGTGACTGCTGCAGACAGGCTCCTTCCACATGCTCTCATACACCTACATATATAT
ATATTTTCTCTATTTGGAGAGTAATACACTGTAGGATTGGGTGGTTTCCTGCATTGAGATCCTTCTATTAATATATCAATGACTCTGCCAATTT
CATACCTGAAAAAGTGGTAGGTTAGTTATAGAAGATCTTTAATATTCCTTTGAGGTCTAAGATTCTGTTAAATATTTTTGTTTAAATTGTGTAA
AATACATATGACATAACATTTATCATCTTAACCATTTTTAAGTGTACAATTCGGTGGCATTAAGTACATTCATATTGTTGTGCACCATCACCAT
CTCCAGAACTCTACATCTTGCAAAACTGAAACTCTGTAGTTATTAACAATTCCCCACTTCTCTGTTGCCCCAGTTCTTTTTTTTTTTTTTTTTT
TTTTGAGACAGAGTCTCGCTCTGTCGCCCAGGTTGGAGTGCAGTGGTGTGATCTCAGTTCACTGCAACCTTCGCCTCCTGGGTTCAAGCAATTC
TCCTGCCTCAGCCTCCCAAGTAGCTGGGATTACAGGCTCCCACCACCATTCCCGGCTAATTTTTTATATTTTTGGTAGAGACGGGGTTTCGCCA
TGTTGGCCAGGCTGGTCTCAAACTCCAGACCTCAGATGATCTGCCCCCCTTGGCCTCCCAAAGTGCTGGGATTACAGGCGTGAGCCACCGTGCC
CAGCCTATTCCCCCAGTTCTTGGTAAGCACCATTCTACTTTCTGTCTCTATGAATTTGACTACTTTAGGTACCTTGTATTAATAGTCATACAGT
ATTTGTCCTTTGTGACTGACTCATTTCACTTAACAGAATGTCAACGCTCGTCCATATTGTAGCATGTTTCAGAATTTTCTGACTTTTAAAGGCT
AAATAATATTCCATTCTTTGTCCTGCATTTTGTTTATTCATTCATCTTTTGATGGACATGGGTTATTTCTGCCTTTGGCTGTTGCAGATAGTGC
TGCTGTGAAACATTCGTGTACAAGTATTTGTTTGAATATTGAGTATATTCCTGGGAATGGAATTCCTGGGTCATGTGGCATTTCTGTGCTTAAC
TTTTTGAGGAACTGCCATACTATCTTTTCCACATTGGCTGCACCTTTTTACGTTCCTACCAGGACCACACGAGGGTGCTAGCTTCCCTACACCT
TCACCAACACTTGTTATTTTTTGTTTTATTGCCATCCTTATGAGTATGAATGGTATCTCATTGTCGTTTTGATTGGTATTTTCCTAATGATTAT
TGATGTTGCATATTTTTGCACGTGCTTATTGGCCACTTGTATATCTTCTTTAGAAAAATGACCATTCAAGTTCTTTGCCCCACCCCCACGTGTT
TTTTTTTGTTTTTCAGATGGAGTCTTGCTCTGTCGCCCAGGCTGGAGTGCAGTGCCCCAATCTTGGCTCACTGCAACATCTGCCTCCCAGGTTC
AAGTGATTCTCATGCCTCAGCCTCCTGAGTAGCTGAGATTACAGGCGTGCACCACCACGCCTGGCTAATTTTTTGAATTTTTAATAGATACAGA
GTTTTGCCATGTTGGCCAGGCTGGTCTCGAACTCCTGACCTCAAGTGATCCCTCCGCCCGCTTTGGCCTCCCAAAGTATTGGGATTACAGGCGT
CAGCCACTGCGCCCAGCCCCTATTTTTAATTTTATCTTTTTTTTCCCCTCCACCTTAAGACTCAGACTTTGCCCATTTTTTTAATTGGGTTGTT
TATTGACATGTAGGAGCTGTTTATATATTTTGTATGTTAACCCTTTATCAGATACATGATTTACAAATATTTTCTCCTATTCTCTGAGTTGTCT
TTCACTCTCTTGAAGTGTCCTTTGATGCACAGAAGTTTTTAAGTTCAGTGAAGTTCAGTTTATTTATTTATTGAGACAGAGTGTCTCTCTGTTG
CCCAGGCTGGAGTGCAGTCACGTGATCTCGGCTCACTGCAACCTTCACCTCCCAGGTTCAAACGATTCTTGTGTCTCAGTCTCCTGAGTAGCTG
GGATTACAGGCGTATGCCCCCATGCCTGGATAATTTTTGTATTTTTAGTAGAGACGGGGGGGTCTCGCCATGTTGGCCAGGCTGGTCTCGAACT
CTTGTCCTCAAGTGATCCGCCCACCCCAGCCTCCCAAAGTGCGGAGATTACAGGTGTGAGGTACTCTGCCCAGCTGCAATTCATTTATTCTTTT
TGTTACCTGTGTTTTACGTGTCATATCCAATAAATCACTGTCAAATCCAATATTGTGAAGCTTTTCCCCTATGTTGTCTTCTAAGAGTTTTATA
TTTTTGCTCTTATATTTAGGTCTTTGATCCATTTTGAATTAATTTTTGTGTATAATGTAAAGACCCAGTTTGATTATTTTGCCAACACTATTGT
TGAAAAAACTGTTCTTTCTCCATTGAATGGTCTTAAGCATCTTGTTGTAAATCATTTGACCATATATGTTGACTTTGGTTTTATAGTAATACCA
TTGTTACAATATGGTTTGATACATGTAATTTTTATTTGATGTTTCTGAACTTTGTTTAAAATATTGGACTTGTTCCATGGCTGAATGATTGATA
ATAAAATAATAAATAAAATATTGTACTTGGAAAGTAAATATGAGGGTAGTAAGTTAGTTATCTATTCGAGTAAAAGAAACTTGAGGCATAGCCA
GTTGCAATTTGTGAGCCTGAGTTGGATCCTGGTCTATAAAATAAATTTAGATAAAATAGGGAAGTTTTTAATATGGACTGGGAATTAGAATTAT
ACTAGGAGATTATTTTGTTACATGTGATAATAGTTTTGTGGTTATGTGGAAGAATATTATTTTTATAGAGATACTTCCTGAAATATTTAGTGGT
GAAGTATCAAATCTGCAACCAACTTTGAAAATGGTTCGGCCAAAATAGGAAATGTAGCAAATTATTAACCTTTTTTTTTTTTTTTTTTTTTTGA
GATGGAGTCTCACTCTGTTGCCCAGGCTGGAGTGCAGTGGCACAATCTTGGCTCACTGCAACCTCTCCCTCCTGGGTTCAAGCAATTCTCGTGC
CTCAGCCTCCTGAGCAGCTGGGACTACAGGTGCGCGCCACCATGCCCAGCTAATTTTTGTATTTTTAGTACAGACGGAGTTTCACCACATTGCC
CAGGCTCCTGACCTGGATCACGACCTCCTGACCTCGTGATCTGCCCGCCTCGGCCTCCCAAAGTGCTGGGATTACAGGCGTATATGGTGTTTAT
TGTATTACTCATTTTTTTCTTATGTTCGACCATTTTAAAAATACATTTTCAAAAAGTTGTTCCCACTGAGCTTTAGATTCACTTATCAGTGTTT
GCTAGATAGTTCCAGGTAATGCGTAAGTACCTTAAACTCAACATGCCCCAGTTAAGCTATCATTTCCTAGTCGTCTTCCTTTATGTCCTTTCTC
ATAGGGTTGTTTTCTCCTCTACCTTCCATCTGACGTCACCACTCTGAGGAGGCCTTTTCCTTACAAACAATGTAAACAATCCCTCTGTCATGTA
TGCCATTGTGGTAAAACAACTGTTTACATTTTCAAGTCCCTCACTTGTTACTGAGCTCCTTCATTGTTGATCTCTTCTGTCCAGTATTCTTAAA
CTGAGCTGTTGAAATGTATTTTCCTTTATTCACCACAGTATTTCATCTTACTTGTGAGCGTTGGCTTTTTCAAAATGAGAGACACTGGTTCTTT
TGATCCAGGCCTTGAATGTGAGGCCCAGAAGTCATTCCAGAGCACTAATCAGTTGACCGCATAATTATTTCGCATTTAATCACTGATGATCCCC
ATGTTAGACAAGGCGATCACTAAATGAGGCTGTGCTTTGTCTTTTAAAGGTGGGAAGAAGAGCGCTATCCTGAAGGCATCAAGTGGAAATTCCT
AGAACATAAAGGTCCAGTATTTGCCCCACCATATGAGCCTCTTCCAGAGAATGTCAAGTTTTATTATGATGGTGAGTTCTTTCAAGGTTCTTGA
TTCTTGGCCAGGAAAAGAAATCTGCTTCTATTTAGGGATAGAAAACAAGGAAGGATCCTATGTAATAGATAATCCTTTTTATTTCATTTTGTTT
TATTGCCATGCAATTTTGAGGAAGGGGCATCAGGTGTTAACTCTTAAAGGCTCATTTGCTGCTGAAAAAGTGCCATGGAAAAGGGGCTATTCAT
GTCTTCCCTACATAGTCCAAAAAAGGTTTGTCAACATGGGTAACAGTGTGTGGTCCACAAGCTGAAGGTTTTGATCTGAACTTCATTTCTAGGG
AGCAACTCTTCGCAAGAGTTATTAGAAAAGCCTCACTTGAATGGTAGTATTTTATGTTAGCAATATAACAATGCAATCGGACAAGCTGCTCATC
ACTAGAAACAGTATTAGTATATGCTCGAGGATTAATAGTCTGCCATCCATCTCTAGATAGGGCTGGTAGGCATCTGTCACCTATTCTGTGTAGC
ATAACACTATGCCTTTGATATATGTGCAATCAAGTCTCATGAGCTGCTTTAAAGAAATGCCCTGCCTGGGTCTCTTAGAACCCTCAGAATTGTC
CCTTATCTGTCGTTGGTATTTGTATCCTTCGGTTTGAAGCATTTGAGGGTAGCCTTTGACATGCCTCCTGTTGGCAGTGTAGCAGTATGTAGAT
GGTAATAACTGATTTTCTTCATTCCATGAGTTTTATCCAGAAATGGTTAGACCATTGTTCCTCATACCCTTATGTGCTTATGACTCACCAAAGT
GGATCTGAGCCCATTTAACAGTTGTGGGGTGGGCTTCTTTTCCCTTTCCCCACCGTCCCTCTTCTTCTTTCCCCTTTCCCAACCATCCTTCCCT
CTTCTGTCCCCTTTCCTGACAGTCCCCCTTCTCCTTTCCCCAGCATTCCTCTTCCACCTCCACTTGTCTTACCACCGTCTTTTGGCCACTGTTA
TTTCACCACCCCATTTCTCCACCATTTTTCCCACCCTCAGCCCCCCACTGTTTTCTTCTCACCCATCCCACCGCCCGCACTGAAAAGAAAAAGT
TCTGGGGTGGGATTGAGTACTTCTCAACCAAGTGGTCTGCTGACCAAACTTTGAGAAACACTGGGCAGGATAACTGCTGAAAAGCATCAGTGCT
GAACACTCAGGCATTCTGGTGCATCTCTCTTCCCACCAGTAGTGGCCTTAGGTCCACCAATGCTTGTAGGCCTAGGTACTACTTTACACTAAAG
GGCTGGGTCTGCAACTGAAGAAAGTGCCAAACTTGGCAAGCTGAAATTTGTTTTTTAAAATGCCATATTTATTAGCCAAGTGTGGTGGTGCACG
CCTGTAGTCCCAGCTACTCGGGAGGCTAAGGTGGGAGGATTGCTTGAGTCCAGGAGGCAGAGATTGCAGTGAGCCTACATCGTGCCACTCCCCT
CCAGCCTGTGTGACAGAGTGAGACCCTGTCTCAAAAAATAAAATAATAAATAAATAAATAAAACATATTTATTGAATCTATTTGTGGTCAGACA
CCAAGACCCAGCAGTGGATAGTACTACTGACTTAGACCCTGATCTTGGGGAGTTTGCAGCCTAACAGGACTCCATAATCTCAGAGGCAAATGCT
TATGTTATGTAAACATTTTGACCTGTTGACCAGAGAGCCTAAGACAGTTCTTTCAGCCCTCACGCTGGTCATTCCTGGAGACTCTTAACTATAA
CTCAAAGCCAAAACTCCTAATTAAGAGAAAGGGCCTAGAACCTTCAAGAGCACCATCTCAAAGCGTAGGTATTCCGTACGCCTGCTCTGGACTT
ACCAAGAGCTTTAGGATGCTCAGCTGGTAAAGCCTCACTTTGAGGTTCAAAGTGATGGGAGGGAAAGCTAGATTCCCTAGTCCTGTGGTTTCTC
TTCCTTTTCAAGATAGGAAGCAGAGGCCTGGTCTAGTTCACAGGTTGCCATAGTCCTTTCCTCCCATTCTTGCCCAGTCCTCCTTTACAGGATC
CTGCTTTAGGGCTCCAGCATTTTCTCAGCTGGTCCTCTGCTGGGATTTCTTCTCTTTGATTGTGTGAACTCTTACTCTGCAGTAAGTCAAGAAG
TCATATTTGACTTTACTTCAAACTGATACAGTAAGTGCTGCCTACTCTCTCGGGAGGAGAATTCTTAGACTCCTTTAGAACTTGCCAGGGAGGT
GGGGCATGGGCTAGCTGAAGCAGTGAATTACTGCCTTCTCTGAGGACCTTCAGTTTACAGCTTTGATTCAGCAGTTTCCTTTTCCTGGAGCTCA
GCCTTTTCCACAAAGTGTTAACATTGCCAGGAACCTTCCAGTCATCCTCCCACTACTCTGATGCAGGGAGCCATCATTTCATCTTTTGACAGCT
CATCTAAAATAGGAGGAGTCACAGTTGCTTCTCCCTCTATTAGGACAGAATTCAGTAAAACAGCCCAAAATCTCATTTTTTTTAAATTTATTTT
TATATTTATTTATTTGAGACAGAGTCACACTCTGTCACCCAGGCTGGAGTGCAGTCGCACAATCTTGGCTCACTGCAGCCTCCACCTCCAAGGT
TCAAACGATTCTCATGCCTCAGCCTCCCAAGTAGCTGAAATTAACACGCACGTGCCACCACACCTAAGTTTTGTATTTTTAATAGAGACAGCGT
TTTACCATGTTGGTCAAGCTGGTCTGGAACTCCTCACCTGAAGTGATCCTTGCACCGCCTCCTAAAGTGCTGGGATTACAGATGTGAACCACCA
TGCTCAGCCACATTTTTTTTTAAATGGTGTTAACAGTTTACATTTTTTTCTTGCCATTTTATTTCATTTATTTTATTCCTAATGACTTAATGTT
TTCCTTTATATTGGAAACATGTTACAGTTTTCTAAAGAAGTTCAGGGAATCTTTTAATACACAGCCTATCTTCCCCGAAGTGTCTTTCTTCCAA
AGCAGAAGCTTCCGGGTTGATGGGGGCAGCTGCGCCACCTGTTGGCCTTTTAAGTTAATGGCGTCCAAAGTGCCTTCTTTCTATTATAACTGTT
TCAATATTGTGCTACAGTTCTCTACACCCCTGCAAAGTCAGATTGCATATCATTATGAGATAAGATGTCTTCTGAGTTTTGAAATAAATGGATT
TCTGGTTAAATTTTAGTTTTCTATAAAGAATGTGGGCAGCCTTGGTGAATTATTAATACCTGTGTAATAGTAAGGTGGTGGAATTATAGTTAAC
CTCATTTTAAGTATAGCCAAATGCTCTTAGTATAGCATGGATTGGTTCGAGGTGATGTTGTAAAGTTGGATTTTTAAGAAACAATCGAAATTGG
CACTAAGAGTACTGTCAAAAGCATTTGTTTGAAAAGTGGATTAAATTCCCAAGTACTTTTCTTTCATTGCTTTATTAACTAAATCAGGTATATA
AAACCCATAGGCCCTATTCAGATTTTCTTTACCAGTTAGCAGCTTTCATTGTGAAAACATCTGAGTCCCTTTAGACAGCTTTTTGTTTATTTGA
GACAGTCTCAGTCTGTTGCCCAGGCTGAAGTGCAGTGGCGTGATCTTGGCTCACTGCAACCCCTGCCTCCTGGGTTCAAGCGATTCTCATGCCT
CAGCCTCCTGAGTAGCTGGAGTTACAGGCTCCCACCACCACGCCCGGCTAATTTTTTATATTTTTAGTAGAGATGGGTTTTCGCCATGTTGGCC
AGGCTGATCTGGAACTCCTGACCTCAAGTGATCTGCCTGCCTCAGCCTCCCAAAGTGCTGGGATTACAGGCATGAGCCACTCCTCCCAGCCCCT
TTAGACAGCTTTTACATAAACAATTGGATACACCAGGAACCCCAGTCATCCCTTGTGCTGTGGATGCATCCTGGTTCCCTGAAAGAGGCAGCAT
CATGGTACCCATGTTCCAGAGCAGTGTGTCGCAAACTCTAGTGTGCATCCAAGTCTTGTTAAAAATGCAATTTCTGTATAGGATTCAGTAGGTC
TAGGAGGGGGCCTGGGATTCTGCATTTCTAAACTCCCACATGATGCCAATGCTGCTGGTCTGTGAACCACTTGTTGAATAGCAAAGTTCTAGAA
GAAGCTCTTATTAGGCTTTTCATATGGCAAATCTGGGCTTGCCTAGTATGGATATATATTTTTTTATTCATATTCCACCCACCTAACTTTGAAG
AAGAAAGATGCCTATTTCTTTTCCCCACAATTCCTTTCTTCCACTCCCTGCCCTATCCCTTCCTCCCCTTCTTGAACAGAGGCTGCTTACCTTA
GCAGTGGCAGGAGTCCCTCTGAGGATTTGATTTTTAAAAACCATTGAAGAGGTGTAAAGGACCCTGCTTGCTGAAAGGAAAAGGAAGAGTGACT
GGGCCTCCCCCAGCTCATCCCTGTCCTGAGCAGGCACCTGGGAACAATTAACTGGCATAGTCTCCAGAGAATGACCCAGGTAAATATATCCTAA
AGAGAAGGAGAATTTGTACAAGTAGATATAAATCAGTATGTTGTCTTTGTTGTTGTTGCTACTATGTGTCACCAGACCTTTATATACCATGAGA
AGGCAAACCATTATTAAAGAGAATTCGCTAGCCCTGGGTATTTATGCTTAGAACATGAATACTATACCTCACTTTTTGGAACCACTTTTTTCTC
TAGGTAAAGTCATGAAGCTCAGCCCCAAAGCAGAGGAAGTAGCTACGTTCTTTGCAAAAATGCTCGACCATGAATATACTACCAAGGAAATATT
TAGGAAAAATTTCTTTAAAGACTGGAGAAAGGTACTGTAGCCCATGTCTTAATATCCTAGTACCTTGCAAAACAATCAAGTACTAAGTAATAAA
TTATCATTTTGCAAAACATTTCCTGATGTAAAATTTGAGTTGTATGGATTTTGTTGTATTTAAACTTGCGTATTTTTTGTCTTCATTTACTATA
TTATGTAGGGTTTCTGTCCAACAAGAGTACTTGTATATCTTTGCTATTGGCTCTCATGTGATCCCACCTAGGTGGGGGTTATCAGATTAGGCCA
AACAGAAAACCTCAACTCCAGTTGCGCACAGGAAAGTTTGTCGCTGCAGGACTGCCCCGTGTCTGCTGTTGCAAAGAGAAATGTTAATCTGATG
TCCCTCTCGCTGTTAATTCTTGCTCATTTATTTTAGGTACTTTCTACTCTTGAAACCACATATACTATATTTGCCTAGGTGATTTTTTAAAATC
TCATTATTCTCAATTTGGATAATGTGGACAGAGGCAGCAGTAAAGTTTACTTCCTGAACTAGCTCAGAAAAAGCAGACTTAATAAATGAATAGA
ATAGGGGTAATGTTTCCAAGAGAGAATCAGTATGGGATTTCAAACTTGTATTCATAATGCCTGGGGGCTGTGTGCCACGGAGTCTAAGAAACCA
CATAAGAATTCATTTTTTTCCATGATAAGTCATTTTAAAACATTTTAATATTAGTAAGTACATATTTTATAATAAAGTAATACAACTGTATTTT
TTTGTTTTTTCAAAATTCATTAATTGAGATTGGCTACTTCCAGAAACCTTTGACTGAAAAAATGGTGCTTGGGTGTATTTGCAAAGAAACCCAA
GGACTTATTAGTGTATTTTCGTTGTTTTTCTTTCATCTCCCCATTTTCTTTTGACTAGGAAATGACTAATGAAGAGAAGAATATTATCACCAAC
CTAAGCAAATGTGATTTTACCCAGATGAGCCAGTATTTCAAAGCCCAGACGGAAGCTCGGAAACAGATGAGCAAGGAAGAGAAACTGGTACTAC
AGAATTTATTAAACCTCTGGAGAATTCTGGAATTGTCATTCGTTCATTTAACTTTCTTCTTGGTTCTTATGACACAACATTAACATCAGTAATT
TCACATCATTCTATGCTAAAGACTTAGAGTTCTCAAAGTCTAAATTTTCTTAAAGGTTTTAGTTAGACTGAAAATTCTGAACAAGTACTTTGCT
CAGTAGCTCTGTACAGGAATCTTGGTGCTTCAGTGACCTAAAAACCAGTCAGTAACTCCAGCCAATAATATAGTAATTAAGGATGGGCAGACCT
GTCCCACACTGGCTACTGGATATTAAGGATTTAAGACAGATGAAATCATATTGGCCATGTACCTCCCTGCTCTTTGATCTATTTTACTCTGGAA
TCTTGTGATGCTCATCCATCTCCATCTTCCAGCCTTGGCCATCCTCATTACTGAAGGATCTTTCTGGTATCCAGCTGGGATGTCATTTTTCTTC
TACAGGCTGCCTGTTTTTGTAAAAAAAAAAAAAAAAAATTTATTGGAACCAGCTGCCTCTACTTGTCTACATATTATCTACGGCTGTGTTTATG
CAACAATGACAGAGATGACTAGTTGCAAAAGAGAGTAGTTGCAAAAGAGACTGTGTGGCACTCAACCCTAAAATATTTTCTATCTAGAAAAAGT
TGTCAGCCCCTGAACTAAAGGAATCCAGGCTAGAATTTCATTTTTACCAATCGGAAGTCCAGAAGTTAAGGATTTTAAGGAATTCTTTGCACTT
CAAATTCCTGTAGTTTCAGTATTTTAAAGGACTTGCGTGTTTACGTAATTCAGACCATTGTATTGATATAACTCATTGTTTCATGGTACTTATT
TGGTCACTAGTTTAAAAATTAATGCATTACTATAAGAATGTTAATTGCATCAGACTTTGTGTCTCAGGTAAATCTTATCCTCAATATTCCAAGT
TAGTGAAATTTAGTATATTATTTACTTCAGGGGCATTTTGAAACTTAAAAGATCACTTTGGAGCTTTGTTCTTAGAATATTGATCCAAAGATAG
ATTTTATAAACATCCCCTTGTCAGAGCCTCTGCCAAGGACACCTGCTGCCAGCAAGGTCTGAATCTGTTCCCACCCACAAGGCAGCACAACTTA
GTTAAACTGAATCAATAAGTATATAAAACGCAGAGTCAATAGAGGGTAAGGAGGCTCAAAATCTCCCCCACCATCCCTCACCTTTTTGGAGCAA
GGATCATAGTATAGAGTGTATATACCTGTATACCTACAAAAAAATTAGTGACAGATCTGTCTGAGCTTAAAAACCCACGTTTTTAAGCTGTTTA
ACTGCTGTTTAATTAAATGGCTTGAGCTATTCTTTTATGAGGCTCTATCAGAGACTCTTCTCCATTTCCTGTTTTGATAGTACTCAGTAAAACA
GTATGAATTTTTAAAGTACATTTAAAAACACCCAAATAACATTTACAAACATATATCCTTTAAGACACAAGAATCAGTTTTGAATCTAGTTGAA
ATCAATCCTTATAGGCAGATCTTTTTGGGGAATTTTATGATATTTAAGTTTGAGAAAACCACTAGAGGTTTTCCTTTATTTGAATTTATTTTTT
AGTCATTTTTCAGACTTTCCTCTACCTTGACTTAGTCTCCCACAAGACATAAAATGCATTAGTAGAGAACAGCAATCTGAATCTATTTTGAGTA
CTTTCTTGCTGTCTTCCAGAAAATCAAAGAGGAGAATGAAAAATTACTGAAAGAATATGGATTCTGTATTATGGATAACCACAAAGAGAGGATT
GCTAACTTCAAGATAGAGCCTCCTGGACTTTTCCGTGGCCGCGGCAACCACCCCAACATCGGCATGCTGAAGAGACGAATCATGCCCGAGGATA
TAATCATCAACTGTAGCA2GTGAGCTCGCACTTCATCCTATCGGCCAAAAAGGGGGTGGACGGCGTCCTTTATTGTACTTCGGAATATTTCCCA
GGTTACACAGGACTGTTTGGGAAGGGAGATACTTAAGAGCAGGACAGTATTTCATGCGTAGGTCTCCAGATGTTTTTGAGGTACAGTTGTCTCC
CCTCATCCACAGGGGATATGTTCCAAGACCCCGAGTGGATGCCTGCAACTTCGACCCAATTGCTGTCACTTGGAACACATTTCTGTTGATGTCT
TCCACCCCCAAATTTAATGTCTTTTCCATGTTTATTAAGTACTTATCACATAGAGTGGCTATAACTTTTGCAGTCTGAGGCACAACAGCAAAAC
TAGCACGACTTTCTTTTTCCTTCTTCACAATTTCACAGATGGAAGGTTAATTCTTACCTTAGATCTTAGGAACTTCACCATACAGTTTTTTTTA
TTAAGTCGATAATTTCCACCTTTTCATTTAAAGCACTCTGTGCAGCTTCTGTATGGCATATCCAAATTGCCAACATCACCACTTTCACACTTTG
GGGCCTTTATTAAGTAAAAGAAGGATTACTTGAACACAAGCAATGTAAAACCATTAAAGTCAACCTGATAAATAAGGCAGCTACTAGGTTACCC
ATGGGCAAGTCACATATACAGCATGGTATGTTGTATATATGGTTCACGCATCCCAGGTGGCACTACAAGAGATTTCATCATGCTCCTCAGAATC
ATGTGCAATTTAAAACTTACAAAATGTTTATTTCTGGAATTTTGTATTGAATATTTTCGGATGACAGTTGGCTGAGGGTAAGTAAAACCATGCA
TAAGGTGGGACTACTGTATTGACTGTTAAGAAGGAAACTTGGAAAATTATGCTCAGCAGATAGGTCCACTTGGGGTCATGAAAGGTGAAATTAT
TCCTCACATCTTATTTCACTATCCTCCTGCTCTGTTATTTCCAGAGATGCCAAGGTTCCTTCTCCTCCTCCAGGACATAAGTGGAAAGAAGTCC
GGCATGATAACAAGGTTACTTGGCTGGTTTCCTGGACAGAGAACATCCAAGGTTCCATTAAATACATCATGCTTAACCCTAGTTCACGAATCAA
GGTAAGGGGATAGTTGAGAGCTGCACTCGTTCATGCTGCTGATAACCTTTTTTTGTTGAAATGTAACGTTCTCGTCCTCTAGAATCACTTTGAC
AAATTAAAAATCCTCCCCATTGAAAGAAGGCAAGTACTTTCATAGTTAGCATTATGGTGATCTTCCTACAGTGCCTCCATTTTTTTGGTCACAT
TGTAATCTTATTTATTAATTCACAAATATTTACTGAAATCCCAAATGTGCCCCACACAGTATACATTTGATGCTAGGAATTATGTTGGTAAACA
AGATAGACGTAGGCCCTGCCTTCATATTCCTTAACGTCCAGTGGTCAGGTGTGTTGAAAGTATGGTGATTGTATATTGGCCTCTGCAGATGTCT
TCTACTTCCTCACCTCTCTTGGTTATGAGAAGGGAATGTAGTTTCTGTATATCCTGAAACAAGTTGTAAAATCTAATGTCTTACATACTTCTGG
CTTCCTGCAGTCTCATCTGGGTAGGTTCACCAGGGCCAAATGAAATGTGTGTGAAAGCACATCATAAAACTGTGTTACATACCTATGCACTGTT
ATTTATTGATAGTGTTATTGCAAGGAGATAACTGCCTTGGGGGAAATCAGTTTTTGTTTGCCAGATAGCTATGTATATCTAGCTTTGGAGAGTC
CCACGAACTTCTTCCCTCCAACATACTATGAAATATAAACCTAACAGAGACAAAAACACAGTCAAGGAATTAGTTCTATGTAGACTGTTAACTG
TTTTATGTGCAGGACACCTGGGACTAAGACCTTCTTGATCCTCGTTCATAGGTTATAACATCTGTTAAAATGTGAAAGCCACAGCTGAAAAAGG
CTATGTGAATAGGCAAGACTAAACTGTACATTTTCTGGCTTGTAGCCCTGTGTTCTTCTGTATATCACTACTTTTCTAATGAAAAATACCTGTT
TATATATTTTCTCCAATTATAAAATATACTTTTCTGGCTGGGTGTGGTGGCTCAAGCCTGTAATCCCAGCACTTTGGGAGGCCGAGATGGGCGG
ATCACCTGAGGTCAGGAGTTCGAGACCAGCCTGGACAACATGGCGAAACCCTGTCTCTACTAAAAATACAAAAATTAGCCAGGCGTGGTAGTGG
GTGCCTGTAATCCCATCTACTTGGGAGGCGGAGGCAGGAGAATCGCTTGAACCTGGGAGGCAGAGGTCGCAGTGAGCTGAGATCACGCCACTGC
ACTCCAGCCTGGGCAAGAGACTTAGACTCTGTCTCAAATAAATAACTAAATACATAAATAAAAATAAAATGTAGTTTTTGTAGTTTAGATAAGT
ACAAAATCAGAAAGCGAACACTTATGTTGAGCAGACTAGTCATTTAAAGAAACATGTATTCCTCTTCTGAAGTATCGCAAAGCTGTATATATGG
CTACAAAAGCAGAAAAATAATAGTTTTAGCCCTTAAGGAACTTATAGGCTGGTGGGCAAATCAGGTAAGAATTCAGTTGTATTAGAAGAAAGAT
AAAAGTAAAATGCATTAGATAAACAGAGGAGTTATTTGCAGCACCTTAGAGATCAACAATAACTTTATGAAGAAAAGTAGGTTATGGTTGGGTC
TTAAAAGTAGTATTTCAAACAACAGAGAACTCTGTCGTGGACAAAGGGGAGGGTGGTAAGCAGAGTATGTTGAGGATAGGGGTTGAGAAAAGGT
GTGATAAAAAGAATTATCAAGTGGTGACAGTTGCACAACATTGTGGATATACTACTAAATGTCACTAGTGGTAAATCGTATCAATGCATATTTT
ACCACAACTTAAAAATGATCAAAATAACAGAGTTACTATTAAAAACATTTGCACATAAGTTAACATTTGGATCTTCTAAGCATACCGCACATAC
ATATACTAATGCCACCATTTATAGATGAGTAAATTTAGTCTCTGGGAGGCCCAAAAATTTGCCCAAGATTCAGTGGCTAGTAACTCTAACTTAG
ATCTTCTTAGAATCTAACTTAGATCTTCAGTCTCTGCATGCTTTCCTGTGACATCCACTGCTAAATGTGGCCAGCTTTAAATGCCATGTTAAAG
AGACTGACCCCTTAAAGGACTTTGAACAGTGGAGTGTCTTGAACACAGCTTCACTTCAGCAAGATTTACAGGCATTATATGTAAATCACATCAG
TAAATATGAACTGTTATAAATGCTGCAAAGGAAAAAGTACAGGGTGCCATCTGAGAGTATACCAGGGTACCTGATCCAGCCTCTGGGGTTCAGG
GATCGCCTTTCTGAAGAGGTAACATTTAAAGGATAATGTAAAGAAAAAATTTTTTTAAATTAGTACCTGAAGATCTGGCTGGCCTCACTATATA
GCTTCCTCTCCTGCCTCTTTCTTCCCTCAGAACGACCAAGCTCTTGCTCACCTCTGGGATTTCAAAAGGCTACTTTATTTTCTCTATTAGAAAC
ACCCATTCTCCTATTCTTTCCAGGGATACAATCCTATTCATCCTGTAAATCTCATCTTAAATGTTAACTCTTCAGAAAGGCCATCCCTGAATCC
TAGAGGCTGGATCAGGTACCCCGGTATACTCTCAGATGGCACCCTGTACTTTTCCTTTGCAGCATTTACAGCAGTGTAAATCTACTGGTGTGAT
TTACATATAATGCCTGTAAATCTTGCTGAAATGAAGCTATGTTCAAGACAACTCCACTGTTCAAAGTCCTTTAAGGGCTCAGTCTCTTTAACAT
GGCATTTAAAGCTGGCCACATTTAGCAGTGGATGTCACAGGAGAGCATCAGGAGCATACGCTCTAAGAAGATTGAAGAGTTACTAGCTGTTGGA
TCTTGGGCAAGTTTTAGGGCCTTTAAGTTTAAAAGGAAGCCACCAACCTGGGATTTAAAGAGGGAAATGACACCAGATTTGTATTTTGGAAACT
CGTTACATAATTGATCATTCTGACTGCTGCTCTGCGGAGACTGGATTGGACACAAACAAGAAGTAGAGAGATCAATGGGAAGCTGTTGCATACT
CCAGGCAAGAAACAGCGATGACTTAAGACAAATGATGGCAATGGGGATGGAGAGAGGTGACAGATACATAATGTATGCTGAGTACTTGTAGAGG
TAAGGATTAGAGTTAATTAATTGTTGGAGCCTCTCTGAGGGCTCAGATCCTTGTTAGATAGACCAGGTGAAATCCCTCAAGAGCAGACAGCCAG
TCAATTTTATTCCAGACAATATCCAGCTAACTGCTATACACAATAGACATTTGTGCAAGTTTCTGGATGATGATCATGGTAGTGAACCTAAAAG
TCACCCAGAGTTAGACTCTGAACCTTATGCTCAGGAGAAAGTGCCTGACTTGAACTGCAATGGGATCCCAATAGGAGTCATTAAAGGCCTGGAA
AAGTGGTGCCATTAGGAGAAAAAGAAATGATTTCTTGAGCTTGCTCTCAGTTCTCTTTTAGGCTGTCTTGTACTCAGCAGAATAGTGAGATCTT
CAAAGGTTGGGGTTTGATAGTGCCTTGAATAATTTTTAACTTTATATTGCCAGCGGAAGAAGCATTCTCTTTTTAGATTTAAAAAATGTAGATA
CAAATATTAGGGGTTTTATTTTTAGTGAAACATTTCAAACATACAGGAATAGATAATTATGTAATGAACACTCGTATGTCCACCATCTGGCTTT
GTAAAATCTTAAAATTATGTCTTATGTGCTCAATTGTTTTATTTCATAAAAGATACTGATAAACATAGCTGAACTCACTTGTATACCATTCACC
TTCTTCCCTGTAGATTACTATGAACTCGGTCTTTTTATTCTCATACATATTTTTTGTATTTTTGCAGTATATTTATCTGTTCATAAACAATATG
TAATTTTACAATATGTAACACACTAGTAACATACTAATTTAAAACTTGTTTTTAGTTTACAATATGTTGTAAACTATTGTAAGCTAAAGACATA
TTGTACAACCTATTGTAAAATAAAAACAGGTTTTACTTTTAAATTAGGTATGTTACTGGGATCATTCTGCAACTTGTTTATTCCTCTCCAGCTT
TGATTTTGTGGTTTTATTATCTTAACCTACACTTTTAATTAATCCATTTTATTTGTTACATGGTATTCTATTATATCATAAAAACTTATCTATT
CTGTTGGTTTTTGTTGTTCGTCATTTGAGACCATGTCTTCGCTCTGTCACCCAGGCTGGAGTACAGTGGCGTGATCTTGGCTCACTGTGACCTC
TGCCTCCCGGATTCAAGTCGTTTTGGTGCCTCAGCCTCCTGAGTAGCTGGGATTATAGGCGTCTGCCACCATGCCCAGCTAATTTTTGTATTTT
TAATAGAGACGGGATTTCACCATGTTGGCCAGGCTGGTCTTGAACTGACCTCAAGTGATCTGCTCACCTCAGCTGCACAAAGTGCTGGGATTAC
AGGTGTTACCCACCATACCCTGCCTCTATTCTCTTGTTAAGAGGCATTTAGCATGGTTATACAGTCTCTTGCCCTCATAAACAGTGCTGGAAGA
AACACATGTTTCTTGTGTATATGAATGAAAATTTGTTTTATACATTAGATATTTCCAAATTGTTCTCTTAAGTACTTCAGTTTACATCATTACT
CTCCTCCTCCCTCCCCTCCCACCCCCACCCACAACAGTATTCCTCTTTTTCCATATCCTTGCTAATGTTTCTAAAGTTTTGCTTTTTACATTTG
GGTCTTAGATCCACTAGAATGTATTTTTTGCATTGGATGAAGTTGAAACCTAATATATTTTCCAAATGAGTAAACTGTTGTCACAGAACTATTT
AGTTGTATTACCTCCTCTCTTGTATATCAGATATATCTACATATATGTCAGACTGTTTCTGGGCTGTCTGTCCTCTTTAATTAGTTCGTGTATC
TGTTTCTGCATCAGTAGCATACTGTCTTAACTACTGTAGCTTTATAAAGTCTATTGAGTAGGACAAGTTTGTTTCATTCTTCAAAATTGCTTTG
GCTATTCTTGGCCCTCTGCTGTTTCATATTAACTTTCAGATAAACTTGTCAAATTCTAATGAAAACTGTTGATAAACTTGTTGATTAACAAATT
CTAATAAAAACTGTTGAGATTTTTATTGGAATTGCAATACATTTATAGATTAACGGAGAAAGATATTGACAATACAATTGAGTTTCCAATTCAC
GAACATGTTATACCTCTCCATTAATTCATGTCTTTTGAATGTATCCACCAATATGGTTTTGTAATTTTCTTCATAAAGGTTTTACATTTAAAAA
ATTCTTATTTTTAAGTGATCTTATAGTTTTTATTGCTAATGTGAATGAGATTTTTTTCCATTATGTTTCTGTTGGTTATTCCTGAAGTGGTAAT
GCTTATAATTTTGGGGTGTTGGTCTTGTATCTGGCAGCAGATACAAGAGGTGCTCAGAGTTGTTCAGGGTTGCTGAACTCTTAGTTCTAAAAGT
GTCTGTCATTTGGGGTTTCTATCTAGATAATTTAATTATCTATAAAAACAGTTCTTCATTTTCAGTTCATATATTTCATATTTCTTTAAGTTTT
AATTTTTATTTTTAAACACAATTATCCATAAAACCCTAACCCTTTCCCTAGTCAACAGCAGTCACAGCCAAATGTTTTATTAATTGCTATACTC
AGTGTTTCTTGTATCTCATACCTTCTGGGGTTTCTTGTCTTGTTGAAATACACCCTTTAATGTTTCTTTAGTGAAGACCCAACAGTCGCACTCA
CTCACCTTTGTTTACCTGAAAATTTCTTTATTTTCATCTTAATTCATAGTCTGTCTTTTCTCCAGTCAAGGAAGTGTCTTATAGGGAAGATTCT
GGTTTCACTATGCTGTATCCAGGGATATATATGTATTTATAGATAGACTTTTAATCTGAGGACTAATGTATTTTATCCTACAGTATTACCAATC
ATTATTTCTTCCATAACTTCTACACCATTCCTTTTGTACTTCTTTTTTACAGTCCTATTAGATGAGTGTTGACACTTTTCAATCTAGACATCTT
TTTTAACTATATTTTCATACTCTTTGTCTCTTTAGGTCTCATTTTTTAAGTTCAGGGGAATATTTCATTTTGGCTGAGTTGTAGCACCTCACTT
CCAATTCACTAATTCTAATTATATTTAATCTACAAGTTATTCCATCTATAATTTATTTCAATTACCACTTTTTGTTTTCAAAATTTCTAATTTT
ATATCTGATTTTGTTTCATTTTTGTTTTATAATTTCATGTTCTTTCTACATTTTACATCTTTTTATGCATACTAAACATACTCACTTGAAAGTC
TTTGTAAGATTGTTCTATAAAATGTTACCTGAAGTGAATTCATGTGCTAATTACTGTTGGCAGTTTTTCTGAGCCATTTTCCTTGTGTCTTTTG
AAATTTCTGTTTGTAAGCCCTTTTAACTGGGAGGTGTTTTTTGCTTATCTACTGTTCTCTTTTCTTCCCCTTCTTCTCCTTCTCTGTTCATTAC
TTTTTGTCTTGGAGGGAGTCTCTTAGCCCAGCTCAAGCCCAAGCTAATAAGGCCCCTGACTTCAAGTCCCCCCTTCTTGCATGGACAAGAAGTG
ATCCTCCTGTCTCAGCCTCCCAAAGTGCTGGGTAGGCGTGAGCCACCACATCCAGCCTAGAGCACTTACATTGCTACATATTTGGAGCAAACAT
AATTAAAGTATGACTTACTGTGACCCAGCAGTCAATAACAGATACGTTTTTGTAATAGAATGTTGAAGGGTCGTAACTGGTGGTTTTACTTCAT
GGAGGGCTTTAGGTCGGCTATATGGGAAGCTGCCATCATGATGCTTTTGTTCTACAGATTTCTGCTCAGCCTCCAGAGATGTCTCCTATTCTAA
GAGTCGTCTTTGTTTACTCTTGTGTCTATATTTTACTTACTTAATTCCCTCACATAATAGTGTATCATTGTATCCCATCTGTGCTCTATCAAGA
AATGTAGCACAAGCATGATGTGAACTATGAATTTTCGACAATTTAAAAAATTAAAGGAATCATTCAGGGGTAAAAAGCACAAAGTTGTAGAACA
GCTGAAGTCTGTTCTTTTTATGTATCCTGTCTTTGCCCCCTTTCTGGAAACTTGGATTGGCCTACTTGCAGTCACTGTCACAGTTACACTACTT
CTAGCCTCAAAGGTCATTGAAAGGATTGGGATTTTTGAGAACCACAATAACACTTGCTCTAGGTACTGAAAAGGTTGTCCTGAACTCATTCATA
TCTTTTTATGTTCATCCCAGTTTGTTTTTGCTTTTCCCCCCAGGATTTTGATTACACAGACATCTTGCAAGCATATTTCGATTCTTTCTCTCCC
ACTCTGTGCCTGGTGCCCGTAGGATCTGAAATAGCATTGGAATGACAGCTAGCCTTCTGGAACCTGCAGTTTCAACCAAAGAAACTCTGATTCC
AGATAACCCAGGAGTTTCTTATCCTGCATAATCTAGCAGTTTACAAAAAGGCTGCTTATGGGTACTCACTGCTAAGTACAATCCACTGCCAAGT
TTGGAAGCATTACCTGCTCTCACAACCCAGGGAAAGTAGCCCTTAGTAAAATTCAGGTTTTTTCAAACCAGCCTTAACCTTACAAACAGAATTT
ATCATCATGATTTTTCTTTCAAATGCAGTACAGCAAACTTTAGGATCAATGTTGACATCGTATTGCGTTTAGTCCTGTTAAAATACTCTCAGTG
CCAAATACACACTTCTGCCTTTTTTCTCTTTAGAGTCTAGAATTCTGTGGGGTTCCAAAAATGTATGTGCTTACAACCTTTTATATGTAACCAC
CTAGGGATTAAAGGGCTAAATGTGAAGGAAAGTATCTTTACAATATTCATCTTGGGAAGCTCTCATTTTGGTGAGTTCTATTAGAGAAAATAAG
ACACAAGCTCATTAAAAATATATATATATTCTGAGCCTGGCATCTTGGTATAAAATTCAAAACGTTTTAAATCTATTTATTAGTAAGACTAACA
TAGACCTAAGTGTGAAGACTAAAATATTAAAGCTTGTAGGTCATATCATGGTTGAATATGACCTTAGAGTAGATTCCTTAGGACAGAAAAACAC
CAACAAGAGAAACAATTGACTAAATGGACTTCATTAAAATTAAAAATTTATGTTCAACAAAAGACACTGCTAAAATAAATACAGAATTTACAGA
CTAAAAGAAAATATTTGCAACATGTTTATCTGAAAGGGCTTATATCCAGACTATACAAAGAATTCCTGTAGCTCCATAATAAAAAAACAGACAT
CCCATCCGCATAATAAAAAGAGCAAAAGACTTGAACATATACTATACAAAAGAAGATACAGGAATGGTCAATAAATAAGCAAATGAAAGAGTGC
TCGACATCACTAATTATCAGTGGAGTGAAGATTTAACCACAGGGAGATACGAATTCCAGTTCTAGGTATTTTTACCTCAAGTGAAATAAAAACA
TTTATCCATCAAAAAACTTGTACAAGAATGTTCATAGCAGTGCTCTATTCATAAGAGTGCAAAACTGGAAACATCCCAGAATAGATAACACATT
ATGGTATAGTCATAGAATATACTATATTTATCAGCAGTTAAGAAGGGAACTACTTATGTATTCAACAACATGGATGAATCTTAAAAACATGGTT
AAGGGCCAGGTCCGGTTGGTGCATGCCTGTAATCCAAGCACTCTGGGAGGCCAAGGCGGGTGGATCACTTCAGGTCAGGAGTTCGAGACCAGCC
TGGCCAACATGGCGAAACACCGTCTCTACAAAAAAATACAAAAATTAGCCGAGGGTGGCGCACACCTGTAATCTCAGCTATTCAGGAGGCTGAG
ACATGGGAATCGCTTGAACCTGAGGGGTAGAGGTTGCACTGAGCCGAGATTGCATCACTGTACTCCAGCCTGGTGACAGAGCGAGACGCTGTCT
CAGAGGGAAAAAAAAAAGTTAAAAAGGCAGACAGAGGAGTCTATATTGTATGATTCTATTTATATAATCTACACTAGGCAAAATTTATCCATGA
TGATAGTAATCAGATTGAAGGATCAGTGGTTGCTTGGTGGAAATTGACTGAGAAGGGAAATAGTCATGTAAGAATACTGGCAAATGTCCTCTTC
CCCATTACAAACGTCTGAGAAAGTTACTAGAAAACAGAATCCAATTTGCCTTCTCACCCCCAACTTTGTAATCTTGAATCCAGATCCTGATTCC
ACCCAACCACTCTGCTGAATGGATAGCAGCTATATTAGAGAGACAATATAGTGGGCCTACTAGTTTTAAAAAGTCATCCAAAGAATGTCATCTA
CAATCAGTTAAATTAGATTTTTACCCATTACTGAAGTGTGATTGAATTAGGGAAAGAGGACAGGTGGTCAAAGGCAGCCTTCTCCAGTGGATTC
CTTGTCTTCTTGAGATGGTTTTGTGAGACCTTGCCTGGAAGACCTAGGTGGATAGTTTATTTTCTAAAAGTAGTCAAGAAAGCTTGTTCAGGGC
ATTGAACACTGCAGGGACAAGCATACTTCTTGGAGGCCAGCCATCTCCTGCTGGATCTAAGCCCGGTGGCTTCTGCCTCCACCTCCATCTCAGC
TTATTTGAGAGAATCCTAGATCCTTGGTGCAGAAGCCCAGCCAGCCTGTGAAGAAAAGTTGGTCTGGTAGTATCCTGTGAATGACGTGCGGGTT
GGGTTTTTCTAAATCTCGTTTACCTGACTCCTACCTTACATTCCAGTGGTGTGACTAAGGAACTGAGCAAACGTAACCCTAGGGGACTTTGAGA
CAGGCTTTGGTAGTCCATGTGCACTAGAGTGAGGGGAAAGAGGCCACTCCAGAGTTGGGTACTCTACTGTTGAAGGTGAGCCTTTTGTCTCCAA
TTTTTCCGTGCATGTGTAAGAACAGTTGTCATGGAGATTTACTACTCACTTGTGGATGAGGACAGCTTATATCTCACCTTTTCTTATTCACCCT
GTTAAATGTCAAAATGTACTAAGACTTAGGTATTTTTTCTGTGTCACCAACTACTAAATCACCCACCCATGATAGCAGGTAGTTACTGAAACTC
TTGTAGCCAAACAAGTGCATAACACTGGTACACATGATGTGAGAGTGCAGATGACTCCTTAGCTTAACTGTGTAAGCTTTTAATTTCTGAGTAC
TTTTAGGATTGAGAAGTGGAAGACATTCTTGCTCTACGTATCTCATGCTTCTGCACACCCAGAAGGCTGGTTGCGCCCCCACCCCACCCCCCGC
CCCCTTTTTTTTTTTTTTGATAGGGTCATGCTCTGTTGCCCAGGCTGAGGCTGAAAGTTATCTTCACCACAGGTCTTATGTCTAAGGACCACGG
GTCCACGTCATAGTCAGGAACCAGGCATGTGTGGGCAGAAATCACTACCACTTTCCTTTGTTGCGGGTGGGGAAATTAAGAGCAGAGATTCTTG
GATTCTTAAATTCCTTCTCTATGTTTATGTCATCTAGTCTGTTTTATGCTGTTTTATGTAATTCCCTGCTTCTGGATTTCTTGAATTTTTTTCC
CCTTCTCACTTGTCATAATTGGCTCTTGCTTGTGAGTCTATTTTCATTCAGCTTGGTAGCAGAAGTCTTCTTCACTATTGTTTAGATTCAGTTT
TCACATGGTTTTAGTAACCCTTCATTGGGTTTAGAATCAACTTTTAACCCTTCCACCAAAAGCTCTTATTTTTTTAAAGGCTTCCCAGAAACAT
AGTTTCTCTCAATCTCATCAAGAAATGCTTTTAAATGCAGAAGTTAGAAAATGGTGAATAATTATGGCTGTTCCTGGAAGTCATTGTGTCCTTA
GTAATTACATGTTGTAGTCTTTGGTTCTGCTTCTTTGACAGCTCCACCACACTGTAATTTAATGGAATTCACTTAGATTTAATGGAATTCAGTC
CTGGTAGAAAACTGCTATTGGGTCACAGAGTTGGCAGCAAGGCTGTGAAATTGATGTGGGAATGGGGGTGATTATGTGCTTTGTGTTGACAGAA
GTTAGTTTTTGGGACATCCCATGTGGGTTTTTCCCAGGAACATAAACATTTTAGAGAATTAGAGCTAGAAACTTTCTTCTGGGAAAGCCTTAGA
AGTCAGTTGTGCTTGGCAGTGAGATGGGACAATGTGCATTCTTAACAGGTTAAATGTGCCAAACTCTCTCTTCAGTACTATTCTTTTTTACGTT
TGCTTAAGGTCAAGTTCCTCCTCTTTTGTTCAAAGTCTTGCTTTGTCACCCATGGTGGAGTGCAGTGGCGTGATCTTGGCTCACTGCAACCTCT
GCCTCCTGCGTTCAAGCAATTCTTGTGTCTTAGCCTCCCTATTAGCTAGCTGGGATTATAGGCTTGCGCCACCTTGCCTGGCTATATTCAAAGT
CTGTCTTTACTACACTGTCCCAAAGTAATCTACATTTGGGTTTGGGTCTTTACACGGTGAGAAGCACTGGCAGAAATACGAGACTGCTCGGCGG
CTGAAAAAATGTGTGGACAAGATCCGGAACCAGTATCGAGAAGACTGGAAGTCCAAAGAGATGAAAGTCCGGCAGAGAGCTGTAGCCCTGTACT
TCATCGACAAGGTGAGAGCATCTTCCCATCGGCATTGTCTAGTGTTGAGCTTAACAAAGGGAGTTTCTGCTCTGCCCCAGGCCCTGTGCCACAT
ACTGTATATCACAACTCAACATACATATATTTGTATGTAAAACTGAATCAAAAGTTGCACAAAACAATGCTTAGCCTTACTGTGAGCAGTAGAT
TCTGATCTTTTTTTAACTCTATTTCATTCCTTTAGAAAGTTGGTTATAACTTATAAAATTGTTTAAATAAACCATGCAGGTATCACCATTATAT
AGTTTTAAAGACCACGCTCTAGAGGAGATCAATTTCTTGGTACTCACGAGAAGAAAAAAAAGTTCATGTTTTTCCCCCAGTCTTGTATTTTAAC
ATAAGTTCTGAATTGTCTCAAAATTCACACTAGCTTTTTGAGTTTGATTGCCCAGAGTAGCAGCACTCCCTCATTTGCTGCCTTATTTAATTTT
TTTTTTCCTTTTCTCCATCCCTATTTCAGAGGACAGAATAGAAAGAAAAGGAGCATGAATATTTAACATCCCTATAGATTTCATAGTGAGTGGG
GGAGGAAGTAACACTGCAGAATCCAGAGTTCCCAGGCCATGCTGACTGATGCTGTGGCTTCAAACAGAATTGGGAGGCTTCTGTTTTGGATGCC
TAGAAATTGCATAAGGGTCAAAAGGCTGGGCGCAGTGGCTCACGCCTGTAATCCCAGCACTTTGGGAGGCCAAGGCAGGTGGAACACGAGGTCA
AGAGACCATCCTGGCCAATGTGGTGAAACCCCATCTCTACTAAAAATACAAAAAAAAAAAAAAAAATAAAAATTGGCGTGGTGGCGGGCGCCTG
TAGTCCCAGCTACTTCGGAGGCTGAGGCAGGAGAATGCCATGAACCCGGGAGACGCAGCTTGCAGTGAGCCAAAATTGCGCCATTGCACTCTAG
CCTGGCGACAGAGCGAGACTGTCTCAAAAAAAAAAAAAAAAAAAACACAGAACGAAATTGTGTAAGGATCATGTCTCTTCCATTCATGCTCATC
TTTTCTTTCTTTCCTGGGCAGCTTGCTCTGAGAGCAGGCAATGAAAAGGAGGAAGGAGAAACAGCGGACACTGTGGGCTGCTGCTCACTTCGTG
TGGAGCACATCAATCTACACCCAGAGTTGGATGGTCAGGAATATGTGGTAGAGTTTGACTTCCTCGGGAAGGACTCCATCAGATACTATAACAA
GGTCCCTGTTGAGAAACGAGTAAGTTAATGTACCTGTACTGTCTGACTTGTTTTCCATTATTCAACAAGCATGGGTTGACTGCTTTTTTGTGTG
CTTTGCACTTTCCTGGGCACCAGCAAAAGTGACTTGAGACACGCAACATGGCACATCCCTGTAGACCCAGCTATTCAGAAGGCTGAGACAGGAG
GATCACTCGACACCAAAAGTTTGACCCTGTAGTGTGCTGTGATCACACCTGTGAATAGCCACTCTACTCCAGCCTGGGCAAAATAGTGAGACCT
TCCCTCTCATATTAAAATAAATAAAAATAAATCACTTGAAAGAGGGGACAGGTATTATTTGATATCTTGTGTACCTCCTTTTAGTCACTCTTCC
GCCCTGTGCTACCTTCCTGGTAAGGAGTGGCCTTCTCCTATACCAGTATTCAATGGACCATGCTGTAGAATAGGCCCTTAGAGCAAACTCAGCA
TATGATTTAACTATATCTTAGTATCCCCTGGTCATGTGGTACATTTGTCACTTTTCATCTTTCCTCAGCCTTTCTCCAACTCCTGACTGAGTGA
AAGGTTAATGTTAGTCCCCACATCTCTGAGCCCATCACTGAAGAGGGTACTATAGCTCTGTAGTCCCAACCTAATCTTTTCCATAGTATTGGGC
TTGAACCAGACACTTACAGTGTGCAGGATCCTAATAATCCAGCCCACCAGCAGTCCTCTTCTTATAAAGACACCTCTTCTCATAAGGACACCAT
AATCAGTAATCAGGTTTTGCTGGCTGTGGAAGTACTCTGGACAGATTAGATAACAGTATGTATCTATGTAAATTTTTGAAGCTGATACTGTGTA
AATGTAAGGGAATATCTATTTTTAGAACATACACACTGAAATGTTTACGACCATATTATAAGTAACTACTTATAAATGTACTTTAGCACCATAT
TATAAGTAAGTACTTATAAATGTACTTTAGGACCATATTATAAGTAAGTCCTTATAAATGGCTCAGAACAAACAATTATGTATATATATCTGTA
AATATACACATGGAGAGAGTGAGCATACATGCACACTGTGCAAATGATGAATGGGGTAAAATGTTAACAGTGAATCTCGGTGACGGATGTATGC
GTGTTCCTTGTGCCTTTTTCTTTGCAAGTTTCTTTAAGTTTGGGGTTATTTCTAAAGTTAGCATTTTTTTCAAGAGTAATAATTAGGATTCACT
TATATCTTTTAGGTTTTTAAGAACCTACAACTATTTATGGAGAACAAGCAGCCC
HUMAN SEQUENCE—mRNA
CAAATGCGAACTTAGGCTGTTACACAACTGCTGGGGTCTGTTCTCGCCGCCCGCCCGGCAGTCAGGCAGCGTCGCCGCCGTGGTAGCAGCCTCA
GCCGTTTCTGGAGTCTCCCGCCCACAGTCACCGCCGCTTACCTCCGCCTCCTCGAGCCTCCCCAGTCCCCGTCCCGCCCGCACAAGGCCGCTCG
CCGTCTGCGTCTCCCCCACGCCGCCTCGCCTGCCGCCGCGCTCGTCCCTCCGGGCCGACATGAGTGGGGACCACCTCCACAACGATTCCCAGAT
CGAAGCGGATTTCCGATTGAATGATTCTCATAAACACAAAGATAAACACAAAGATCGAGAACACCGGCACAAAGAACACAAGAAGGAGAAGGAC
CCGGAAAAGTCCAAGCATAGCAACAGTGAACATAAACATTCTGAAAAGAAACACAAAGAGAAGGAGAACACCAAACACAAAGATGGAAGCTCAC
AAAAGCATAAAGACAAACATAAAGACAGAGACAAGGAAAAACGAAAAGAGGAAAAGGTTCGAGCCTCTGGGGATGCAAAAATAAAGAAGGAGAA
GGAAAATCGCTTCTCTAGTCCACCACAAATTAAAGATGAACCTGAAGATGATGGCTATTTTGTTCCTCCTAAAGAGGATATAAAGCCATTAAAG
AGACCTCGAGATGAGGATGATGCTGATTATAAACCTAAGAAAATTAAAACAGAAGATACCAAGAAGGAGAAGAAAAGAAAACTAGAAGAAGAAG
AGGATGGTAAATTGAAAAAACCCAAGAATAAAGATAAAGATAAAAAAGTTCCTGAGCCAGATAACAAGAAAAAGAAGCCGAAGAAAGAAGAGGA
ACAGAAGTGGAAATGGTGGGAAGAAGAGCGCTATCCTGAAGGCATCAAGTGGAAATTCCTAGAACATAAAGGTCCAGTATTTGCCCCACCATAT
GAGCCTCTTCCAGAGAATGTCAAGTTTTATTATGATGGTAAAGTCATGAAGCTGAGCCCCAAAGCAGAGGAAGTAGCTACGTTCTTTGCAAAAA
TGCTCGACCATGAATATACTACCAACGAAATATTTACGAAAAATTTCTTTAAAGACTGGACAAAGCAAATGACTAATGAAGAGAAGAATATTAT
CACCAACCTAAGCAAATGTGATTTTACCCAGATGAGCCAGTATTTCAAAGCCCAGACGGAAGCTCGGAAACAGATGAGCAAGGAAGAGAAACTG
AAAATCAAAGAGCAGAATGAAAAATTACTGAAAGAATATGGATTCTGTATTATGGATAACCACAAAGAGAGGATTGCTAACTTCAAGATAGAGC
CTCCTCCACTTTTCCGTGGCCGCCGCAACCACCCCAAGATGGGCATCCTGAAGAGACGAATCATCCCCCAGGATATAATCATCAACTGTAGCAA
AGATGCCAAGGTTCCTTCTCCTCCTCCAGGACATAAGTGGAAAGAAGTCCGGCATGATAACAAGGTTACTTGGCTGGTTTCCTGGACACAGAAC
ATCCAAGGTTCCATTAAATACATCATGCTTAACCCTAGTTCACGAATCAACGGTGAGAAGGACTGGCACAAATACGAGACTGCTCGGCGGCTGA
AAAAATGTGTGGACAACATCCGGAACCAGTATCGACAAGACTGGAAGTCCAAACAGATGAAAGTCCGGCAGACAGCTGTAGCCCTGTACTTCAT
CGACAAGCTTGCTCTGAGAGCACGCAATGAAAAGGAGGAAGGAGAAACAGCGGACACTGTGGGCTGCTGCTCACTTCGTGTGGAGCACATCAAT
CTACACCCAGAGTTGGATGCTCAGGAATATGTGGTAGAGTTTGACTTCCTCCCCAAGGACTCCATCAGATACTATAACAAGGTCCCTGTTGAGA
AACGAGTTTTTAAGAACCTACAACTATTTATGCAGAACAAGCAGCCCGAGCATGATCTTTTTGATAGACTCAATACTGGTATTCTGAATAAGCA
TCTTCACGATCTCATGGAGGGCTTGACAGCCAAGGTATTCCGTACATACAATGCCTCCATCACGCTACAGCAGCAGCTAAAACAACTGACAGCC
CCGGATGAGAACATCCCAGCGAAGATCCTTTCTTATAACCGTGCCAATCGACCTGTTGCAATTCTTTGTAACCATCAGAGGGCACCACCAAAAA
CTTTTGAGAAGTCTATGATGAACTTGCAAACTAAGATTGATGCCAAGAAGGAACAGCTAGCAGATCCCCCGAGAGACCTGAAAAGTGCTAAGGC
TCATGCCAAGGTCATGAACGATGCAAAGACGAAGAAGCTAGTAGAGTCAAAGAAGAAGCCTGTTCAGAGACTGGAGGAACAGTTGATGAACCTG
GAAGTTCAAGCCACAGACCGACAGGAAAATAAACAGATTGCCCTGGGAACCTCCAAACTCAATTATCTGGACCCTAGGATCACAGTGGCTTGGT
GCAAGAAGTGGGGTGTCCCAATTGAGAAGATTTACAACAAAACCCAGCGGGAGAAGTTTGCCTGGGCCATTGACATGGCTGATGAAGACTATGA
GTTTTAGCCAGTCTCAAGAGGCAGAGTTCTGTGAAGAGGAACAGTGTGGTTTGGGAAAGATGGATAAACTGAGCCTCACTTGCCCTCGTGCCTG
GGGGAGAGAGGCAGCAAGTCTTAACAAACCAACATCTTTGCGAAAAGATAAACCTGGAGATATTATAAGGGAGAGCTGAGCCAGTTGTCCTATG
GACAACTTATTTAAAAATATTTCAGATATCAAAATTCTAGCTGTATGATTTGTTTTGAATTTTGTTTTTATTTTCAAGAGGGCAAGTGGATGGG
AATTTGTCAGCGTTCTACCAGGCAAATTCACTGTTTCACTGAAATGTTTGGATTCTCTTAGCTACTGTATGCAAAGTCCGATTATATTGGTGCG
TTTTTACAGTTAGGGTTTTGCAATAACTTCTATATTTTAATAGAAATAAATTCCTAAACTCCCTTCCCTCTCTCCCATTTCAGGAATTTAAAAT
TAACTAGAACAAAAAACCCAGCGCACCTGTTACAGTCGTCACTCTCTATTCTCATGGGGATCAATTTTCATTAAACTTGAAGCAGTCGTGGCTT
TGGCAGTGTTTTGGTTCAGACACCTGTTCACAGAAAAAGCATGATGGGAAAATATTTCCTGACTTGAGTCTTCCTTTTTAAATGTGAATTTTAT
TTCTTTTTAATTATTTTAAAATATTTAAACCTTTTTCTTGATCTTAAAGATCGTGTAGATTGGGGTTGGGGAGGGATGAAGGGCGAGTGAATCT
AAGGATAATGAAATAATCAGTGACTGAAACCATTTTCCCATCATCCTTTGTTCTGAGCATTCGCTGTACCCTTTAAGATATCCATCTTTTTCTT
TTTAACCCTAATCTTTCACTTGAAAGATTTTATTGTATAAAAAGTTTCACAGGTCAATAAACTTAGAGGAAAATGAGTATTTGGTCCAAAAAAA
GGAAAAATAATCAAGATTTTAGGGCTTTTATTTTTTCTTTTGTAATTGTGTAAAAAATGGAAAAAAACATAAAAAGCAGAATTTTAATGTGAAG
ACATTTTTTGCTATAATCATTAGTTTTAGAGGCATTGTTACTTTAGTGTGTGTGCAGACTCCATTTCCCACATCTTTCCTCAAGTATCTTCTAT
TTTTATCATGAATTCCCTTTTAATCAACTGTAGGTTATTTAAAAATAAATTCCATCAACTTAATGGAAACTTAA
HUMAN SEQUENCE—CODING
ATGAGTGGGGACCACCTCCACAACGATTCCCAGATCGAAGCGGATTTCCGATTGAATGATTCTCATAAACACAAAGATAAACACAAAGATCGAG
AACACCGGCACAAAGAACACAAGAAGGAGAAGGACCGGGAAAAGTCCAAGCATAGCAACAGTGAACATAAAGATTCTGAAAAGAAACACAAAGA
GAAGGAGAAGACCAAACACAAAGATGGAAGCTCAGAAAAGCATAAAGACAAACATAAAGACAGAGACAAGGAAAAACGAAAAGAGGAAAAGGTT
CGAGCCTCTGGGGATGCAAAAATAAAGAAGGAGAAGGAAAATGGCTTCTCTAGTCCACCACAAATTAAAGATGAACCTGAAGATGATGGCTATT
TTGTTCCTCCTAAAGAGGATATAAAGCCATTAAAGAGACCTCGAGATGAGGATGATGCTGATTATAAACCTAAGAAAATTAAAACAGAAGATAC
CAAGAAGGAGAAGAAAAGAAAACTAGAAGAAGAAGAGGATGGTAAATTGAAAAAACCCAAGAATAAAGATAAAGATAAAAAAGTTCCTGAGCCA
GATAACAAGAAAAAGAAGCCGAAGAAAGAAGAGGAACAGAAGTGGAAATGGTGGGAAGAAGAGCGCTATCCTGAAGGCATCAAGTGGAAATTCC
TAGAACATAAAGGTCCAGTATTTGCCCCACCATATGAGCCTCTTCCAGAGAATGTCAAGTTTTATTATGATGGTAAAGTCATGAAGCTGAGCCC
CAAAGCAGAGGAAGTAGCTACGTTCTTTGCAAAAATGCTCGACCATGAATATACTACCAAGGAAATATTTAGGAAAAATTTCTTTAAAGACTGG
AGAAAGGAAATGACTAATGAAGAGAAGAATATTATCACCAACCTAAGCAAATGTGATTTTACCCAGATGAGCCAGTATTTCAAAGCCCAGACGG
AAGCTCGGAAACAGATGAGCAAGGAAGAGAAACTGAAAATCAAAGAGGAGAATGAAAAATTACTGAAAGAATATGGATTCTGTATTATGGATAA
CCACAAAGAGAGGATTGCTAACTTCAAGATAGAGCCTCCTGGACTTTTCCGTGGCCGCGGCAACCACCCCAAGATGGGCATGCTGAAGAGACGA
ATCATGCCCGAGGATATAATCATCAACTGTAGCAAACATGCCAAGGTTCCTTCTCCTCCTCCAGGACATAAGTGGAAAGAAGTCCGGCATGATA
ACAAGGTTACTTGGCTGGTTTCCTGGACAGAGAACATCCAAGGTTCCATTAAATACATCATGCTTAACCCTAGTTCACGAATCAAGGGTGAGAA
GGACTGGCAGAAATACGAGACTGCTCGGCGGCTGAAAAAATGTGTGGACAAGATCCGGAACCAGTATCGAGAAGACTGGAAGTCCAAAGAGATG
AAAGTCCGGCAGAGAGCTGTAGCCCTGTACTTCATCGACAAGCTTGCTCTGAGAGCAGGCAATGAAAAGGAGGAAGGAGAAACAGCGGACACTG
TGGGCTGCTGCTCACTTCGTGTGGAGCACATCAATCTACACCCAGAGTTGGATGGTCAGGAATATGTGGTAGAGTTTGACTTCCTCCGGAAGCA
CTCCATCACATACTATAACAAGGTCCCTGTTGAGAAACGAGTTTTTAAGAACCTACAACTATTTATGGAGAACAAGCAGCCCGAGGATGATCTT
TTTGATAGACTCAATACTGGTATTCTGAATAAGCATCTTCAGCATCTCATGAAGGGCTTGACAGCCAAGGTATTCCGTACATACAATGCCTCCA
TCACGCTACAGCAGCAGCTAAAAGAACTGACACCCCCGGATGAGAACATCCCAGCGAAGATCCTTTCTTATAACCGTGCCAATCGAGCTGTTGC
AATTCTTTGTAACCATCAGAGGGCACCACCAAAAACTTTTGAGAACTCTATCATCAACTTGCAAACTAAGATTGATGCCAAGAAGGAACAGCTA
GCAGATGCCCGGAGAGACCTGAAAAGTGCTAAGGCTGATGCCAAGGTCATGAAGGATGCAAAGACGAAGAAGGTAGTAGAGTCAAAGAAGAAGG
CTGTTCAGAGACTGGAGGAACAGTTGATGAAGCTGGAAGTTCAAGCCACAGACCGAGAGGAAAATAAACAGATTGCCCTGGGAACCTCCAAACT
CAATTATCTGGACCCTAGGATCACAGTGGCTTGGTGCAAGAAGTGGGGTGTCCCAATTGAGAAGATTTACAACAAAACCCAGCGGGAGAAGTTT
GCCTGGGCCATTGACATGGCTGATGAAGACTATGAGTTTTAG

TABLE 32
MOUSE NOMENCLATURE
ICSCNM Dusp10
Celera mCC15978
HUMAN NOMENCLATURE
HGNC DUSP10
Celera hCG25031
MOUSE SEQUENCE-GENOMIC
GAGAGAGATCAGTTCTTTGTCTCAAGCAGACCTTTGGCTTACTGGATTAGGCTTGTCCACACACTTGAGAGTGGTGGTCTCTTCATTCAAAGTC
TGTCAATTCAAATGTTAATCTCATCCCCAAACATCCACACACAGAAGCAAGCTGAGTCACATCTGCTCACACATCTGCCCCTGGCGGGCCTGTT
AACACCGTCTGGGGGGAAGATGGCACTGGCTTCAACTGAAGGAAGAGAAATCTGCATCCTTGTAAGCAGGCTGCAGTGTTTGATTTTGGACTTC
TAGGTTGGGGATAGAGTGTCTCTGATGAGCATGTCTCTAAACATCTGTCAGCATTGATACCCTCAATTTCCCTAGAGGTTTCTGGGCGTTTTTG
TCTCAGAAGCCCCTAAGGTCTGGGTGGATAAGTGATGACTGTCATTTGAATCAGGACACTTGCCCTAAAACCCACCTTCCTGAGTGTCTGTTGA
GTACCATCGCACTGAGTGTCAGATACAAGAATGCGTAACCACCAAGGACTCAATAGACCGGTCCATCCTTCTCCAGGCCCCGTGATAGTCAGTT
TGACTCAAGACAGATCTGAGTTGGATAACATATCTTCCACTTGTATCGACTCCTTGCCTTCTGCATTCTCACATATATCCTTTCTAAGCCAGGT
ATTAAGAAATTAAAAACTCAAAAAACAAGTCCAGTGCTAGTCAGCTTTCCTTTGTTCCTGTGACTTGATGCCTGAGAGAAACAGGCTAAAAGAG
AAAGGCTTGTTTGGCAGGTGGTTTGACTCTACCTTGTTCCTGGGCCTGCGGTTACCTCAGAAGGCGCTCACCTCTTTGTGGCCAGGAAACAAAA
AAGAGAAGACAAAAGGAACTGTCACAAAACACTTCTTTTAATGGCCCACTCCTAGTAACCTACACACTCCAACTAGACTCCACACCAACAAGTT
TCTCCCACCTTCAGGCAATACCTTAAACTTCTAAGGAATAAGAGAGTTAATCTGGATAAGAGGTCAGGACCCCTACAATCCAATCACCTCCTCA
AAGCTCTTGGCCCTCCAAGATCAGAAAACAAAAGAAAAGTAGACAATTTTTATAGAGTCAATTTAAGGAACCCTGTAGGCTGAGAGCTAAGGAC
AACATCCACAAAATCACACAGAGGATGAACTACACAGCACAGGATGCTCGCGGGCAGTTGGTCTTATCTCGGGGTCACTTGCTGGTTCTGCTGT
TTTCTGGCTGAATGCGCTTGGGTAGACACTTTGTCTTTGCCGATACCCACATCAGTCTTCAGAAGAGAGCTACTTGATCTCTGATGCTAAGGTG
GAGATTTAAATGTCTTTGTTTGTAAAACAATCACATGCTGCTCTCAGCCCAGAGAAAGGAGGAAGTCCCATCAAACTTCTTCTCCACACCCTAG
ATCTTTAGAATCAGGTAGTGTCTTAGTCAGGGTTTCTATTCCTGCACAAACATCATGACCAAGAAGCAAGTTGGGGAGGAAAGGGTTTATTCGG
CTTACACTTCCATACTGCTGTTCATCACCAAGGAAGTCAGGACTGGAACTCAAGCAGGTCAGAAAGCAGGAGCCGATGCAGAGGCCATGGAGGG
ATGTTCTTTACTGGCTTGCCTCCTCTGGCTTGCTCAGCCTGCTCTCTTATAGAACCCAAGACTACCAGCCCAGAGATGGTCCCACCCATGAGGG
GCCTTTCCCCCTTGATCACTAATTGAGAAAATGCCTTAGAGTTGGATCTCATGGAGGCATTTCCTCAACTGAAGCTCCTTTCTCTGTGATAACC
CCAGCTGTGTCAAGTTGACACAGAACTAGCCAGTACAGGTAGGTTGATAACTTGCTTGTAAAGACAACAGAGCACATTGAGGCATACTGGGACA
TCCTAAGAGTCTGCTATATCTGCATCCCAAGCAAGAGTTGCTAAGATTACATTTGCAAATTTGGAGAANNNNNNNNNNNNNNNNNNNNCCACCC
CCACCCCTAGTACACATCAAGTCCCTTCAGACAAGATGCATCCTCCCCCACTGAGGTCAGACAAAGCAGTCCATTTAGGAGCACAGGATCCATA
GGCAGGCTGGCTGGTAGGCAGGCAACAGACTAAGAGACAACCTCTACTGTTTTTGGGGAACCAGCATGAAGACCAAGACCATCTGCTACATATG
TGCAGAGGGCCTAGGTCAAGTCAGTGGTCACACTCTCATTGGTGATCAGTCTCTGGGAGCTCGTAAGGGTCCATGTTAGTTGACTCTGTTGGTC
TTCCTGTAGAGTCCCTGTCCTTTTCAGGTCTCTCGGTTCTTCCCCAAGCTTCCCCCAAGCTCCATCTATTGTTTGGCTCTGGGTCTCTACATCT
CTTTCATCTCTTTTTGGCTGCTAGGTAGATCCTCTCAGAGGACAGTTATGCCAGGTTCCTATCTGCAAGCATATCTGAGTATCATTAGTACTGT
CAGGGACTGGTTCTTACCCATGGCCTGGATCTCAATTTGAGACAGTCATTGGTTGGGTATTCCTTCAGTCTCTGCTCTATCTTTGTCACTGCAT
ATCTTGTAGACAGGACACATTTTGGGTCAAAGGTTTTTGTGGGTGGGTTGCTGTCCTTCTCCCTCCACTGAGATCTTGTCTAGCTACAGGAAGT
GGCCACTTTAGGATCCATGTTCCCCACTGCTACGASTTTCAGTTAGASTCAGCCCCATASACCTTCTGGACCCTCTCCCCATCCCAGGTCTCTG
GTATGTCCTAGAGATTGTGTCCCCCTCCCCCCACCCTTCCTTCTCCCTCTCCTTTGCTCCACTTCCTGTTCGCTCATTTGTGTATTTATGGTTA
ACTATCTGAGCTCTCCACTTTCTGTTCCTGCTAAGCCTGTCGCTTGCTTCCATGCCTCTCTGTTCACCACGGTGGACTTTTATCCCTTTGGAAC
TATAAACCCAAATGAACTCCACTCTTCTGTAAGTTCCCTCCGTCATGCTGTTGGATCATAGCAGAACCAAGAGGGCTACATTTCTTTCTTTACT
GTGTGTGTACACATGTTCACATATGGGTATACTCATTCCTGAGGGATGTGTGGAGGTCAGGAGTGAACACTTGGGTTATTATTATTTTTAAAAT
ATGTGTATGGGTGGTGTGTGTGTGTGTCTGTGTGTGTGTGTGTATGTATGTATGTATGTATGGCATGCCTGGAAACAATGAAGCCCAGAGGAGG
GCAGTGGATTCCCCTGGGACTGAAGTTAGGATGGTTGTAAGCCACACTGTGGGTCCTGGGAATCAAATCCAGGTTCTCTGGAAGAGCAGCCAGA
GTTCTTCCTCTTTGSTCTCTGGGATGCCATCTTAAATCCTTTCTCTACCTTACTTTTTGGGGTAGGGTCTCTCACTATCTCTCCAGCTCACTGC
TTCAGTTAGACACACTAGGAAGTAAGCCTCTGGATTGGCCAGTGTTCCTCCTTCAGCTTACACAGTAAGTGCTTTCACCCACTGAGCCATTTCC
TCAGCTCTCAATTACGATTCTTAATTTTTTTTTTTAAAAAGAATATGTGTGTCTTTGTGGGAGTATGCGCATGTGAGTAAAGGTGGTCTTGGAG
GTCAGAAGAAGGTACCTCAGCATCTGGAACTGCCACTAAATTTGGTCATGAGCTAATGAAAGTAAATAAAACTTGAGACATGTGATTCATGTAA
TTTATGTCAAATAGCCCAAAGAGTTGTTTGTGAGCTTTAAAACCTGGGGCTGAAAACATAGCAGAACAGGCCAGGACATGCCCGGGCAGGCCCG
TTGTTACATGTCCTGACTGGCCTAGTGCCTCCCTATCTCCTGCCCTTCTGACAGTCTGTTGATGTTTAAATGGACCAATCATGTGAAACCGAGC
CAATTCCTCCCCCAGCCCCACCCCTTTTCTATAAAATCCCCTAGCTTCCAAGGCTCGTGGTCGAAACCACTGTCTCCTGCGTGAGATACGTTTC
GAGCCAGAGCTCCGCCATTATGGCTCCACCATGTGGTCGACACCTCTGTCTCCTGCGGGAGATATGTGTCAGCCCGGAGCTCCGTCATTAAACT
ACCTCATGCTTTTACATCAAGATGGTCGTCTGTTCGTGATTCTTGGGTGCATGCCGAACGGAAATTGAGTGGGGGTTTCCCCACTAGGTTCTTT
CACTACCACGTGTAGGTGCTAGGGACAGAACTCAGGCCCTCTGCAAGAACAGCAAGCATACTGTAGCAACTGAGCCATTGCTTCAGTTCCCTCA
TCTATGTTTTATGTAAACCTTATACATATAACCTTAAAGATTTATTTATATATGAGTACATTCTTCCTGTCTTCAGACACACCAGAGGAGGGAA
TCAGATTCCATTACAGATGGTTGTGAGCCAACATGTAGTTGCTGGGAATTGAACTCAGGACCTCTGGAAGAGCAGTCAGTGAGTGCTTTTAACC
GCCAAGCCATCTCTATTTTTTTAATACTTTTTATTAGATATTTTCCTCATTTACATTTCCAATGCTATCTCAAAAGTCCCCCATACCCTCCTTC
CCACTCCTCTACCCACCCACTCCCACTTTTTGGCCCTGGCGTTCCCCTGTACTGGGGCATATAAAGTTTGCAAGTCCAATGGGCCTCTCTTTCC
AGTGATGGCCGACTGGGCCATCTTTTGATACATGTGCAGCTAGAGTCAAGGGCTCCGGGGTACTGGTTAGTTCATGATGTTGTTCTACCTATAG
GGTTGCAGATTCCTTTAGCTCCTTGGGTACTTTCTCTAGCTCCTCCATTGGGGGCCCTGTGATCCATCCAATAGCTGACTGTGAGAATCCACTT
CTGTGTTTGCTAGGCCCTGGCCTAGTCTCACAAGAGAGAGCTCTATCTGGGTCCTTTCAGCAAAATCTTGCTAGTATATGACATGGTGTCAGCG
TTTGGAAGCTGATTATGGGATGGAACCCTGGATATGGCAGGCTCTAGATGGTCCATCCTTTCGTCACAGCTCCAAACTTTGCCTCTGTAACTCC
TTCTATGGGTGTTTTGTTCCCAATTCTAAGGAGGGGCAAAGTGGTCTTTGTTCTTCTTGAGTTTCATGCGTTTAGCAAATTGTATCTTATATCT
TGGCTATCCTAAGTTTCTGGGCTAATATCCACTTATCAGTGAGTACATATCACGTGAGTTCTTTTGTSATTGGGTTACCTCACTCAGGATGATA
CCCTCCAGGTCCATCCATTTGCCTAGGAATTTCATAAACTCATTGTTTTTAATAGCTGAGTAGTACTCCATTGTGTAAATGTACCACATTTTCT
GTATCCATTCCTCTGTTGAGGGGCATCTGGGTTCTTTCCAGCTTCTGGCTATTATAAATAAGGCTACTATGAACATAGTGGAGCATGTGTCCTT
CTTACCGGTTAGAACATCTTCTGGATATATGCCCAGCAGCCATCTCTATTTTAACATACCTGCATTTAGAGATTTCCATTGGTAACATGAATAT
TGGTGCTCAAAATGCGTTGAATTTGGAAGCATTTTGCATTTCCAGATTGAGAATGTTCAACGCCTGTTTAGACCTGAAGTCCTGGGATGGTTAG
CAATGTCACACAGTGCTTGTTTCCCTTCCAGTACCCTACCTCAGTAAGATCACCTGCAGCTCCGAGCCAGACCTTGGCAGTGACTTCTAAGGTT
GAAACTCAGTGGTTCGGCTTGTTGGGGGGGCAGGAGGAATGTGGACAGCGTTTAGTGTGGCAACACATCGTACTCATTAGAAGCCATCAGTTCA
CTATGAACTGGCTCATACGTTAGGCATTATCAACACCGTTGGGCCTACCAGAATGCTCTTCTGACATAGAGCAGCAGATATTTGTGACATCACA
AGGCAGTGGCAGTCCACGGGGCAGGAGCAAAGACAGCTTCAGAGAAAGGGTCTTGCTGCCTAAGTGAAGACAAGGGGACTGGGCCTCACCTGTC
CAGCCAGTCACCTCTGTGCGGCCAATGGAGGTCAGCTTGAGTTTTGAAGAGATGTCTGGGCCAAGTCCTGTATCAAACATTGGTTTGGGCTTAT
GCTATCTTTATCTCTGGGCCTCTAMTGATCTCAAGATACACAGACAATACAGATGGAGCTGTCAGGCTCTCTCTCTTAAATGACAAAAGATGGC
TTTGTGGTCTCTTGAGTAAAACATGCCCCTGGCCCCCATCTGTACATCTATATACCTGTGAGGGAGTAACCCTGTCATCATATACAGCTGGTCT
CCACTCCCAGTCACAAGGCCCCACTTCCTTCTAGTTCATCTTGTCAGATAGCCTAGAGGGCCTCTGATATCCTAGTAGAGTGGTTATCTTGGCA
ATCCAAGTGGCTTCAGATGAAAAGCAGCCGTGGCAGGCTCCAGAATGGTGTCATCCAGCAAAAATGGAGTAAATAATCATCTTAGGCGCTCTCC
TAGTCACATGTGAACTAAAAGATGAGTGAAATTGATTTTAATGACATGCTATGAAACCTAGTATATCAAAAAACATAAGCAACACAGACATAAT
ATCCTTTATGTCTTTCACAGGTTTTCATATCCAATGTGTGTTCTACACTTGATGTGTCTTGGTTCCAACCAATCACATTTCAGGTCTTCTTCAG
TCACATGTAAATGGCCACCATTCTACACTACATGTTTGCTCCTTACAGGTAGTAGTGAAGATCACTCCTGCCTCTAGGGCTACAACTTAGGTTA
CTCCTAATGACAGAGTGCAAGATCTGACGACTAATTCAGTCTCAGCAGTTAAAAACAGAACTTCACTTCCCAGAGTTCTTGAGTCTGAGGAGAC
AGATTAAGGCCTCTGGTGAGGGCCTAGCCTCTGCTTCCAAGATGCACTGTCACCGTGTTCTGGGGAGATGATGCACGACATATACACACCATGC
AGAGAGGACACAATAATTCCCTTCCCAGTTCCATCTGCAAGGCCCTGCCCTCATGGCATTACCACCTCAGACAAGTGTCTATCCCTTGGATACT
ATCACACTGAGGACTGAAGTTCAACATGCGAGTGGACCTTCGTGGCCTCTTTCTCCAGGGGCTGCTCCTAGCCCAGGTCTACCCTCGCGAGGGT
TGTGAAGTATTTCCCAGCCGTACTCCTGGGACTTCATCTCCTACTTCCTCCTTTCTATTCCCCAGTGCCTTCCCTATTTCCTGATTGAATGCCA
CCTTACCTGGGGATTTAACTTTGCCAACTCCCTTTCCCTAACCGAATCCGGGAATCTATCTTGGCCTTCAGGGCTATTGAAAGACCTCACCATT
CTGCCACCAAGCACCTGACTTAACCTCTTGAACATGCCCTGTATCAGTCTGTTTTCTGTCTCTGATCTTGGCCACAATTAGCTTTGTCCTTCAC
CTCTAACACACAGCACAAGGATCTGGTAGAAACTCTATCCCTCCTTTCAGTAGCAACAAGTCACAGACCAGCATAAGTGCACTACGTGGCCCAA
GGGCAGTAAAGAGGCTTTACTCTTTCTCTGGGACAAAAACTAAACAGCAAGGTGTTCCTGGGTATCCGATTGTGTTCATGGCCCTTTGCTGACT
GACTGATCCCATACATTTAAACTTAAGGGCTAAGTTTTAGAAAGTATTTTTACATTATGACTCCAAATTAGTAATTGAAATCACCTCCCTTGGC
ACAAAAGCATCCTGGAGTAGTGAGTGGAAAAACCTACCTGGAATCTGACTTCTTTGAGTATAGCTACTCAAACTGCTGCCTAATTTAAAAAGCC
TCCGGATCTTTAGCATATGTTAATGGCTGGTAATTACCATGCATATGTTAATGACATGGAAATTACTCAATGCTTCGGGTTTGATTGATGAATA
ATTTTGTCAGTTTTCCCAATTTTTCAGCCTGGATGACAGACTTTTAGTTAACAGAATTACTATAACTTATTAGTCTGAAACTATAATAATCATC
TCATTAAATGAGTTACATTTCTTTCTCACATTGCCGGCATCAGGCTTTGTGGATCACAATTTACAGGTTGTCAGTACCTTTAGACAGAGGCTCC
TCCAGCCTAAAGCAAACACAACCTTCCCAGCCTTCGGTCAATAGAGAGCCTCGCTCTACTATACCCCGTAACCACTTCCACAAGCATCTTTTCA
TCTTTTTCGGAGCAAGGACGGGAGTTTGCTGTTTACAAATGTACACAGTGATGAACACAGCAATGTGGCTGGACAGACAAGCATCAGACCTTAA
TACCTAGAACTCAAACCGTAAATGGGAACCCCAGCATACATACATCCAGTAAATATTCAGGGATATAGATAACCTACAAAAGTGCTCTGGGAAA
TTCTTTTCTGGGTTCCACGGAAAATGCGCACAGCCTACCTCCTCCACTAGCCTGATCCTGCTAATCCTCAAAGATCTAAGGACCAAAGGGTGCG
ACCTGGACACTCGTGTGCAGACATTCGGGTGTGGGTCCTGTGTATTTGGGTGTGTAGACAGAATGGGGCTAAAGAAAATCCTTTTCCAACCCCA
GTGTGCCTTCTGTAGTGGTGCTCAACTCAAGGGAGATCATGCTGAATCGACAGAGCTAGTCAGCGTAGGGTGATCATGGTTGGACACCTTATGA
TCCTAGATCAACAGCAATGGTGAATGTCTGGGGCCTAAAGCAAACGCCTCCATCATTAAGGACATCTGAACCTGCCACGGAGCTGATAAGTGTT
TTATTCCACTTCAATTACTTCACATTTTGGTCTTCCTCTCTGAGGGGGTAGGGAGGTATAAAAAGCTCTATTGCTTTAGGATTCTTTAACGCAA
NNNNNNNNNNNNNNNNNNNNTTTCTGGCAAGGTCGCTAGCAGGCTTGCTCAGCTTCCCACTTCTCTTCACCGGTGACATACCCCGACCAGTAGT
GACCCCTCTGATGGACAAGTTCGGGGAATCGGGAGAGCTGGCGTGGGCCCTTCCATGCTCCTCAGGCGCCGCTCATTCTGGCTGTTCGTTCCCG
AGCTCTCGCTCCTTCCCGCCGGCGCTTGCTGTTCCAGGCTGGCGCTCCTTGGCTCGCCGAGGGCCCTGCGCCACTGAGGACCGGGGGCGGCCCA
GCACCGGGGGGCGGATGGACTGCGGGCTCCAAAACATCGCACAGGGGGAGGTGGCCGCGGGAGAAGCGGGGACCGCGTTGGGAGGAGGTCGGTG
CCTGGGCTCCGGGGCTTGTGGGGCGTTCGGAGGGCCCCGCCACGCCCAGCTCTGCCCGACGGCGCCCACGCTGCCCTTCAACACCGGCCCCCAG
TGGCTGCTCTCACAGAGCCAGGCGTCCTCCTCCGAGTAGCTCTCCGGAGTGCCGCCTTCGGGATCGGAGTCGCGCTCCTGAGTCCAGGTTGGGG
ACGGCGGAGGACTGACAGGGTCACGGAAGTAGGGGAACAAGAGCACGGCGCGCAGCCGCCGCGCGGGAGAGCAGTAGTTCAGCCGGAGGGGCAG
CGACATCTCCGGGCCGGGCCAAGCGGGGCTGCGGGGAGGCCGGCCCTGTCCACGCTCACGTCCCGCGCGCCGCAGCCACAGCAGTGGAGGCACC
CGGCTTCGAGTCGTCTCCGGCCTGGGGAGTTGGATGGACACTCCACCTGGAGTCAAAAGGCGGATAGTGGAGACCCCGCGAGGCTQATGTCCTC
AACAGGGGGCGGGGGAGGGTCAGAAGAACCAGTTTCAGCCGGAGCGACCAGAGCAAGCCATTGACACTTCAGTCCCAGGAAGGGTTAACCGCGT
CCTGCTGCCTAGGTTTACATGTAAATAGAGAGCCGCTGCTCCAAGCGGGCCCGGGCTAGCACTTTTAAGTTTTTGATTGGTTGCCAATGACATC
ACCACCCGCGCGTTCTAACACACCAAACCCACTAGCGGGTCCGAGCCGGCCTCCCTCCCAAGGCGAGGCCTCGCCCCTTTCTCCGCCCTCCTCG
GCTTCTTATTGGTCAGAGACCACTCTGATGTAGCGGGCGGCAGCCTCTGGGTCTTCATTCGCGTATCGCGCCGTCGCTCGGAGGCAACCCCGCC
CCCTCCAAAGCTTACATTTACGCTGTAGCAAAAGAGAAAGTAACTCTGTGTTGCTGCTGGGGATCAATGAAGCTGAGTGAATGGGGGCGCACTG
CGCGAGCGCATAGCTGGAGCGGGCGCCAGATGGTAGACGGCTACGCTTATTGATGAAGTAAGTGGAAGACATCACGGGTCACTCAGAGCCGGAG
GGTGGAGGGTTCCGTGTGTGTGTGTGTGTGTATGTGTGTGTGTGTGTGTGTGTGTGTGTGTGTGTGTGTGTCAGTGAGAGAGAGAGAGACAGAG
AGACAGAGAGAGAGAGAGACAGAGACAGTGTCAGAGAGACAGAGACGGACCTACGTCAGGTAAGGAAGATCCTAAGGTTTAGTCTTTTGGCTCT
GCTTGCCCTGGGATGAAGAGGGGAAATGAGTTTTGGTTTGGTTAAGATGCTTTATGGCAGTTTTCAAAGCCCAGGCTTTGCGTCATGATGCTCA
GTGGTGGCACGGAATAGGGATGCTACGTGCCCCAGGAGTGGGGAAGGAGCATTCTTTAAGAACTTGGTTTTTCTTCAGTCCCTGTTAGTGGTGG
AAGGAAGATTAGCCAGAAAGGGAGGTAGGGGGTTCGTGCTATTTTTGTGGGAGGTTTCCCGGGCCAGACTGGCTTTGGAGCTATCTGCTGCAGG
ATGCAAATCTCAGTGCGCTCTGTGCCCGTGTGTGAGCAGCAGCACAGTGAAGAGGTACCCGTCCCAGCCTGTGTCTCTGGAATGTCGTTCCCGG
GTTCTCCTCCTAGGGAATAGGCTACTAATCCTGGATGTCCCCTTACCAGGTAGCAAAATAGCAAAGACTTTGACTGCATTCATGTTCCAGCTGG
GACACAGTTAACAGTTTTTGGAATAGTACTCGCTCTGAAAAAAAAAATACTACGCAAGGGAGAGGGAAGGAGGGAGGGAGGGGAGGGGAGGGGA
GGGAGGGAGGGAGGCAGGCAGAAGGTTTGATCCTTTGCTCCCAACCAAAGGTGGGTGAGAGAAACCCGAGTCAGTGGGGATATGCCTACACGGG
TGGGGACGTGCTCCTGGGTGCGCGTGTGTGGATTTTGCTGGTGACACTTTGGCTCGGTCTCAGAGAACAGTTACGGTCATGAATAGACCGATGA
TCCTTGATGTTTTTCCAGTCAGCTCTTAAAGTTCGTTTAGATGAGTGGCTCTTGCTTGGGGTTTGATTTCTCTAGTAGAGGAGTCAGAATTGCT
ATGTTATAGGGTGGCCTTGTGCCTACCGGGAGGAGGAAAGTGATCCATTGCAGGCACCCTTGGAGGAGGCAAAGGCAGCCCCCGTGGGCTCTAG
GTCTGAGCTTCCCAATTAGCCTAACTGTGGTAAAGGGACGAGTGAGGTTGCCCTCTTGGAATCCACACTGTTTCCAAGAAGAATATCAGTATAG
GGTTGAAACATTTTTTACAAAGTTGCCACTCCAGTGGAGATGCAAAGTCTGTCTGTATGACCAGATGAAACACATGCTCTTTTTTGCTTAGGGG
CACAGCATAGAGAAGTGTAAGGATGAGTGGGGTCACACAGGAACGGGCCATGGGTCAGCCTGGGGTGGGGTTCCTAGATCTGACTCCTTGGAAA
CTTGGTTTCTGGATAGTTATTCGTAGTGTAAGTGTGTGTGTGTGTGTGTGTGTGTGTGTGTGTGTGTGTGGTAATGGCTTGATTTGATTTGGAA
GAGTCTAATGTGAGCTCTGAATAGGATGACGTTGAGCTTTTCAAAGGACCCACCAGAATGGAAAGGAAGATTAGCTCCTAGCCATGGAGGCTGA
CTTAGGGAGACACATTCCTTCTGATATGCAGAAGGTTAACATCCAGCCCCCATGTAAATATAGCTAGCTGCCAAGCATCTCCGTGCATCTCTGT
GAGCTCCAACAGTGCTCTTGCTTGTCTCTTTGCCTTGGCTCTGGGGAAAGAAATGAAATACAGTACATGCCAGGGTGGACAGATGCATCCATCA
CAGGTGAAATAGACTTCTCTGCACAGGAGAGGAGGCTCAGAATAAATCTAAGAGCCTCCCTGTCTGCCTTCACTGTTTTTCAGGCCTCTGTGCC
TTTACCTCCACTAAGGCTGAACTTGGATGCATGTCTCTAACATTATGTGACAGGAGGTCTCTTACCGTTTCTAATGTCAAGTCTAATTTTAACC
TTTGACTTAGCATCAGAATGGATGGGGCCCCTCACGTATGCATCCTCTCTGAACAGATCAAAGTGAATTTTAGCCTTTGATAGTATCACAACCT
AGGGCACCTAGAAGGACTTTGACTTTCCTCAGAGATATGCAGAGGTCTTCCCCCGGGGATTGGATGAGACTGGCTTTGTTTTCATGTGACTTTA
TTTTTTTCTTTCTTCCCCTTCTTTCCACACTCTTGACTTCTTCAATTACCAGTTTTCAGCCTCCTCATCCCTCCATCTCCTTTACACGACAGGG
TAGTAGTGGCACTATCTAGGCCTGTCCGACCTCAGGATCTCAACCTTTGTTTAGACTCAAGCTACCTTGGCTCTGCCAGCCCAGGTAGTGGTAG
CCACGCTCCTGTCCTTGCCACCGCCGTTGTGACCCTCAGGCTGCGAATCTCACGTATATGCCCTCATCCAGCGGTCTCTCCCCCCTCCCTGAAT
TGCGGATGCAGCAGCACTAGCTGCTGCACTGTGGCAACCTACGACAAGGATCATCAGGCCCAGACTCAAGCCATTGCTGCAGGCACTGCCACCA
CCGCCATACGAACCTCGACCACCTCCCCTGCTAACCAGATGGTCAACAACAATGAAAACACAGGCTCTGTGTTAACTCCATCGGGTGGGGTGGG
CAGCCCTGTGTCAGGGACTCCCAAGCAGCTAGCCAGCATCAAGATCATCTACCCCAATGACCTGGCAAAGAAGATGACCAAATGCAGCAAGAGT
CACCTGCCCAGCCAGGGTCCCGTCATCATCGATTGCAGGCCTTTCATGGAGTACAACAAGAGTCACATCCAAGGAGCTGTCCACATTAACTGTG
CCGATAAGATCAGCCGGCGAAGGCTGCAGCAGGGCAAGATCACTGTCTTAGACTTGATTTCCTGTAGGGAAQGCAAGACTCTTTCAAAGAGGAT
CTTTTCCAAAGAAATTATAGTTTATGATGACAATACCAATGAGCCGAGCCGTGTGACACCATCCCAGCCACTTCACATAGTCCTCGAGTCCCTG
AAGAGAGAAGGCAAAGAACCCCTGGTATTGAAAGGTAATGACTCTCCCTTCCCTCCCCACCCCCACCCCCCACATTCCTTCCTCAAATAATGTC
CTGATCAGCTAGGTTTCCCTATGAGATTCTCTTACAAGTGGCAGCCTTCTTCTAACCCCAAGGCACCATGAACCAGAATAAAATAGCCTGTCTT
CTTGTCTCTACTTCGAACTTTTTCTTACTCTCTTGGTACATTAAGAAAGTCCAAGTAGCAATGAATTGATATAAGATCTGACTCCCAGCTTTGA
TCTGGCTGCCACTTTGGGTTGCCTATGACATTTTACTTGAGATCCTTTGCAAAAGGCACTTTGTACAACCAAGATGGTATCTCAAGCCATGCCT
TCTCTGCTAGCTCTGTTTCAGTCCTCGAGTGGGTTGGAACTTTTTTGATCTCTCAAGGCCTGACAAATGTCGATGGATCAGAGGAAGCTGCGTC
TACAGGTGCCAGTACTTTGTGGGAGAAACTCTTGCTGTTTCTCACCTGATGGTCTTGATGTTCTTATTGCTTCTAGTCATCTTCAACAAAGGCA
TGCATCTTTATCTTAAAGCCTCATTGAAAGTGTGAATTCTCCAAATGCATCCATTCCTCTCAGGAAGCTGCAGGGGATAGAGATGTGAACCGCC
TACAACCATCCTTCCTGGTATCACCTGACAAGAGTTAGCTTCTGTCTCTTCCCTAGAAGGAGTCATACATCATTTTCCCCTAGTGTAGAATAAC
TGATGACTGGACACCCCCCCCCCCCAAAAAAGGAATAGATGACCCAGTCTTCTCTGAGCTTCTGAGCATACAGTGTTGTCCTGACCAWGCGCAC
AGTCCACTTCCTCATCTTTGTCCTTTTGGGCTTTGAGCATTACTCTTCTGATAATATCTACCTAGTCATGATGGAGAGTCATAACTATCTTCGT
TTCGAATTTTGAGCCATTGTGTGCATGGTGGGTGGGGTGGCGTGGGGTGGAGTCCAGAAAAATAAAGGACCTGGGTAAGTCATCTGTCCACCTC
ACTGAAATACTGACTCTTTTCTAAACATTGAAACAAGAAGATCCTGGAAGCAGCGAAGATTTTCCTATTGCTTTAGTACTGATGTCAGATCACT
GTATGGAACAATGCTACTTAACAATCCTCATTAAAAAAAAAAAAAAGAAGAAGAAGTAGATGGCTTATTACTATAAATTATTTTGTATTATAAT
TATGTTGAGWTTTTAATCAATATCACTCTTTACAGGCCAGACATCATTTATAGTACTTAAATAACCACTATTTAATTTAATCTTCCCTTGGCCC
CATCCCTTGCCCCCACCCCACGTGAACATCGTCATTATCTCCATNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNTTAGCCTGTTGCACTTCCCTTGGTCCCATCTTGTCTTTCTTTTCAC
AGCTGAGAAAACACCATCTACAAATCCCCACACCTGTAACCAATAGCTTTGTAGGCATCTTTTCTGCCTCCTGAAGATAGGATAGGTTTGCTAA
AAATCAACCCCTTTTGTCTGTGTCATCTAAGAAGAGTAAGCACACAACGTAGTTTATGATGGCTCTAAACTCGAACACAGGGCTGATTACGATG
CATAGATCCTATCCTAGCTCTTGCTGTGGAGCTTGGTGGGTAGAGGGATGGCCCATCAGTTGCTGAGAAATGTCTCTCTTGGGCAGTGCTGAGG
AAGAAGAAGGGATTCCTGTACTCTTCTCCCATAGGTACATCTGAGGATCCCTGCAGCCTGGGTTTGCTCATGGGAGTCAGTGTAAAGACTGCCT
CAGTGGGGCGGGGCTGCTGACCTCACCACCAGGCGCCCTCGGCAGCTGCGCTGAGGGCTCATCATTTCGGATTGGTTCGACCCTGGCGGCCGGC
TGGGAAGACGCTCTGGTGGTAGAGGTTGGGTTATTTGTAGTTTTCGGGGGGCTTGTTCCTTCAACTATCTTGGAATTTGGTTGAAAATACATAG
TTCAGGAGTAAAAGGTCTGGTCGTCTACTTTGTTGTCATTATTAAGTGGTACCTTTGTGAGGAATGGCCTAAAATCAGGATCAAGTTAGGGAAG
AGCTAAAGGAGATGGGACATGGAGTCCCCTGTTAGCCTCTTCTCTGCTCAGACCAGAGGTTAATGTCAGAGCGCCCTTGACZTAGAGATGAGAC
GTGCTTTTCTCTCAACAAGTATCGTGTGCAGCTTCTCCTAGGTTCTTGAGTGGATCAAGCACACCATCCTGGGCTGGCTTCTCTTCTGACGTTT
ATTTTAGGATTCACCTTGTTGGTGAGCTGAGATTGTATTTCAGCTAGTGGGGGAGTGGTACCATTTTGGAGTGAGTGAATTTTGTAGGGAGCCT
GTGTCTGCTGGGGTGGGGTGGGGGTAGATGGTTTGACTATATGGTTGTAAGTCCCCAGGCCTTCTTGGTTTATTTTCACAGATTTTTTTTTTTT
TCCCAAAAGATGCCTGATCCGTCTGACQTTCTGGTTCATGTTTGTAAACACCGGTTGCTACCCTCCCGTTCCAACTTTTATGTCCCTCAGTTTC
AATGCCACGGTCTCCATTTTTGGAGCTGCCTGTCGGGCCCGGTGGCGGCTGGGTTGCCATTTTAGTTAAGCAGTGTCAGGTTGGGACGAAACTG
ATCGGAGGCACAGAAGCCCCTGGGATTAAGTTACTGTTCTAATTTATAGTCCCCGCCCCGCTGTGACCTAGGTCTGAGACGGTGTAGTCTCTGG
CTTGTTCTTACCCACTGAAGGATATAGAGCACCAGGCCCTTGTTTCTGGTCAACCTCCTCTGAGTGAGCCACATGAATAGCTCTTTAGAGACTC
AGTCTTTGAAACCATTGTAACCTTTCCCAACCAGTGTTGATTGTTGTGATCATCTGTAACTTGCTCTCCAACCTCAAGGCTCGACAGTCCTTGA
CCTTCTAAGACGAGAGGTGCTGTTCTGTTGTCACATCTGTCCTTTTTCACATCACTGGGGTCAGGGAACTTATTTGTCCTTTATATATGTGGGA
AGGGCATGTTTTCTACAACTCTTTCTTGGTAGCCGTGGCCTTTAAACCGTTATGATCACAATTTTCAAAGTGAACCCCTTTATTCCCAACCAGA
TTAAGTATATGCTTCACCTATTGGCCTTCCCTTCCTTGTTTTTGAAATATTTTATGTCTTATGCTATAGTCTACTGTTTAGAGTTAGTGTTATA
TATTTAATTGAGCTGGGAGTATTTTACAGAGTGGTGTAGACATTTTTTTGTTTTTTTGCCCCCAAGTTAGACATGTAGTGTCATATACCTGCCC
AGGACTGTTCTCCAAATGTGGAATCCCACTGTTAGTGACTCCCTCTTGAGTCTCAGGACACGCCTCCCTCCCCTTCCTGGACACCCTCTTTCAG
GGCCTGTCTCTGCCTGTCCAATTGTTATGTCAGAGTCCAGGCCTGTACTTGCTGCATGCTCTCACATGCGATGGTAGACTGTCTGCTCTGGGTT
TCCAGAGTTCTGCTCTATACCAGAGGCTTTTTGAACGGGTTGTGGGGGTAGGGGTAAGGATGAAAAAGACGGTCAGGGAAGTAGCTTTGAAAGG
TTGATGCAGAGGGGAQAAGTCAGGCAAAGGAGTGAGATTTACACTAAAAACTTCCCCCTGATCTCCACTGTGCATTTTCGTATTAGAAGTACTT
TCTATTAAAATATCCAAACATAGTTGAACTATTTTCACTGTAAGACGGCATGATGAGTATAAATATGTAAGTTCATGTTATATACCCACCCGGA
GCTCCCAAACCTTCCTCTTAATTCCCGAATAAACAGCAAATGTGGTCTAACAGTTTCTCTTAATTAAAATAGATTCTGAAATATTCATTACATG
GCAATACCGACTCTCACGACAGCGAATGGCTACGCCAGGAAACTAGAGACCCTGGGAAAGTCTCCTTTGTCTGCTTTCTTCTGGGGGAGTAATA
TATCCGTCTTTGCTTTGTCTGCTCTTGTAAGACACACCTACACTGCAAACTTATTTTATTCTTGTAACTGTAATTCCAAATAGATCTGTAAGAA
AAAGCAGAGAAATGTAGGGAGAACTGCAGATCAGCAGGCCTCAGCACACTGGGTCCCTCCATTCAGTGTCCTTGGGCAGGCTGCCTAAGTTTGC
CTAAGCCTTTAGTTCTCTGGCTTCCTCACTGGTCACAAGAAGGCGCAGCACTGGTGTTGCACTTGGGATGTTCACTCAGGAATTCAATAAGCGG
AGGCTTGCAGAGTGTTCGGCAGAGCACCCTCTCCACCGAGCTCAGCAATATTTCTGTGCATTGAAATGTGTCAGTCAAAAGGTAGTTTGTCCTC
TTGCTCCCAGGGGTACAACAACGCTCGGTCTAGCTGTGTGCCTGCCTCTGTGTGTGTGTGTGTGTTGCTAGGGCCTAAAAATAACTTGATGACT
CTTACCTAATTTAAGTAGCATACAGTTGTGCCAAGTAATTGCAAAAACATGTTAGGTCAAGGGGCTGAGTAATTTGGGCATACCCTAGTATTAT
TCATTTGTAGGGCAGCTTAGGAAAATGGGATGATAAATCTGTCCAGCTAATGTCCTGGATAGGATAACAAAGTGACTAAACTTTTCAACTTTGT
CTTCTCTGGCCTCGCTTCGGATTTATTTTTGGAAGGAGGATTGTAGTCTGACTATAAAAGGGTAGGTGGAAGGAGGAGGGGTGTGGGCTGCTGC
AACCCTGAGTGCCAGAGAGGAGCTGGAGTGTGAGGGAAGAAGAGGAGCTGATGCTTGCTCATTTGAGTGGGTCCTGAATGCTCCCCCCGAAGGA
CTACAGGCTTGCCTGCCCTCCTTCCTTCATTGCAATCCAAGTGAAAGACAAAGGGACTTCAGCCCGCCCCTTTCCCATCCCTATTCTTACCCCT
CTGCTCCTCATAGGTAGCTTTTCCATGACACTACACTGCATATAGCCCCTATGGCTTTGAGATGCTTCGCTGCTTAAACAGAGGGGAAGGGCAA
ATTGGTCTGAACCCAGGACTCTGCATCATTACCAGGTCAGATCTATGAGCCAAACTGGGTTGAAATGAACTGGGATCAGCCAGCTGGGTTGTCA
AAGATGAGTTCAGGAAACTCTTGACAGTGTGGTGGGTAGTAGTCACGGATTGCAAACCGAGAAACTGACAGGAAAGCAGTCCTCCTCGTTACAT
TTCTAAAGCACATTTCTCCAGGAGGCATGAGTCGGGAGAAGGGTGACTGGGTGTTTCCCCGCTTAGTCTGTTTGTGCCAGCCCATCAGGTGGTC
TGATGAGATCACGAGGGCAGAAGAGGTTCCTGGGCGGAGGCCTCTGTAGCAGGCCGTGTTTCTCTATATCTTGTTGGCTGTGATCAGCAGTTAT
CTCCATTCTCCAAGATTCCTGGAGAGCAGTTTGAGTGTAAATTAGGGCCACACGACAGTGCTTAGGCAACTTTATCCACTTTGGTACCAAATAA
CTAACTACACACTTAACGATATTCTGTTTGTTTTGAGATGGCGGTGACTGTGGAGTTCAGGCCAGCCTGGAACTTGTAACGGTCATCTTTCCTC
TGCTGCCAAATTATGGCCAAGTGCTACTACGTCCAGCTTAAGGATAGAAATTCTGATCCTCAGAGCGGTCTTCTAATCAATTCTGGAAGGCTGT
TGATTCTATGCTGTCACTAGTGGATGATTGACACTCTCTGGNNNNNNNNNNNNNNNNNNNNCCAAAAGATTTTGATTCATTTGGTCTAAGGTAC
AACCTGCACACATGCCAAGATTTTGAAAAGCTCCCCAAATGATTCTCCTGCACAGTGAAATTGGAAAACCATTGATCTAGTTCGAGCTCGTTAT
GCAGTTATGGAAATGAGGCCTGGAGAGGTTTATGACTAATCCAAGGTCACACGCGTGATTAATTTCAGAGCCTTGGTCCTTTGTGCTAAAATAC
GTCTTGTTAGGGCTATAGACTGATGTATGTGTGGTACATTATTACCTCAGAGCCTCCCATGTATAGTGTAAGACATGAAATAAAATAATACACG
TAGTCATTTGAATACTAGTACCTTTTTGGTTTTCAGGTTTCTATTCAGAACAACAACAATAAAATAGACTTAAGTCAAAGGATTTTGGAAAAGC
CGTCCCTGATGGTCGAGATTTTACCTAGGTCCTGGAATATGGGCCAACTATTAGCATCGATTTAATCCCATTTTCTTAGGAACCTAATATTTTA
AGGGAGAGCTAAAAAGTAATGCCAAGGTACATCCAAATAAAGGGTTGGGGAAGGGCTATCTACATGGAAAGTACTTGCTCTGCAGGTGCAAGAC
CCTGGGCTTGATCCCCAGAGCCACGAGCTAAAAAAAAAAGACAGTTCAGCATCTCAGATGCTAACAGCTCGTTGGCAGGTAGCATCATGGAGTA
CAAACTGGTGGGTAGAGAGGTTAGACCATAGGTCGCATCACTGCTAATAGTCCCGTCTGTGTGTGGTGTGTGAAGCACTGTGTGAGTCTGGAGA
CTGTGTTCTGTCAGTGTTTGGTGAGCTTGTGCTTCTTTTCATCCCCCCACCCCCACCCCCACCCCCACCCCCAGCTTGTTTAGAGAATGGCTTA
TCTTCAGGACATGTTATTCTGCTAAGTATCTTTGTGCCATTTTTCTCTGCTTTCTTTTTCAAGAATGTTTCTAGTTTTCTCGACGTTCACTAAG
GGCCTACAGAATTTCTTTCCAGGGCTGCAGTAGGCAAAGGAACGATGTATCATTTTGAATGGAAAGGGAAATATCACATGTTCTTTCTGCTTCT
GCAGTTGTGGCAGACAGGGCCCCCCTGTGTAGCAGAGTGGAACTTTGTCATTTGAGCCTCAACTGTTTGGACATCTTTCTGATAGGAACAAGAC
CTGTGTTTAATTTCACCTCGTGTCCCTTCTGAGGAAATGGGCAAAATGGGCACACGAAGTAAACAGACCACTCAAACGGTGCTTCAGAGACTAG
GTTTGGGCCTGTTTTGTTTTATCTTTTAATTTTTTAAAAAAAATTTATATTTTAAGGACCATTCTCAGGTATCAGGCTAGGATGAAAACAGTTC
TCATTGGTTCCAAAGCCTTTGCTCTTTCCCCTATAGGATGAAATTTCTCTGTATGAGTTTCTTTGTGCTGTTGCTGTGAGAAGATGCTCCAGCC
AGAAGCCAGGAAGGGTTTCTTGGTACTCACAGTTTAAACCGAACATCTCGTCGCCAGTGCGTGAGGCAGCGGGTTCCATGGTGTCTGCAGCCAC
GAAGCAGAGAGAGATGCTTGCTTTCTCGCTTTTCTCCTGGGGCCCAGCCCCACACGAACGGCGATACCCACATTGCATCTTTTCAACTTAACCC
AGTCAAGTAATCCTTCACTCGCATGCCTGGACACTTTTATTTCCTTTTGATTTTAGGTCCCTTCATAACCCCTCTTTGGTAAATTTCCATTGGC
TACATTTACCATATACCATATCTGCTATGTGATCTTACTAATTGAGAAAAATTAAAGAGGTGAACAAACAATGAATGGCCATTTATTTTCAAAG
CCTCTTGTTGATGGCTGAGCCCCTGCCTTAAAACAAACTTTTATTGTAAAGTAAATGAAGGCACATTTTGCATTCAGCTTGCTAGTGCTTTCTA
ATCATGAAGGGTTTTTGTTTTTGTTTTCTGTAGATGTTTTCATCCAGAACTCAAGAACAAAATCTCCATGTTATGTTTGTTTTCGGAATATATC
TATGTCAGATATCATTACTTAACAGTTAGCAAATCTGTATTCCCCTTTCACCGGCAAGACCCGTATTTTATGTAGCTTAAAGAAAACGAAGGCT
GATTTGTCCTTGCATAGGAAATCCGCTGTGCCTGCTTGAAACTTACACAGGAGAGAAAGGCAGTGCCCTCTCAAGTCTTTCCTACCCTTCCCAC
TGCTGGGCTGAAATTCCCAGTGGAAAGCAGGCTAAGGGAAGAATGTTTTATTCTGGCTTAGGGTTTGATGGGQAAAGCCCATCATGTTGAGCAA
GGCCATGAGGACGCTTACTCTTGTCTGGGCTCACCAGACTTTCTCCCTCTTTAACTTCGAGTGGACCTCCAGCCTATGAGGCACGACCCTTACA
ACTTTTTGCAGCATTCTTATAGACACATCAGAGGTATGACTCCTACTGAGTTTCAATCCAGTCAAGTTGACAAGATGAGACACCCTACCATAGG
TCTCTCTCTTGTCAGCTGGGCATCCTAACACATCCCTAGCAAACCATCAGTCCATGTTCTGCCCTGGTAAAGTAAAAGTGCCTTTACAAAGAGA
AAGTTGGTGTAGATTCAATACATTATCTGACTTCTTTAATGTTTTATTTTTGACAAAAGCCGCCTCACAGCCCCACCCCAGTCTTGAATTTCAT
GCAAAAACAAACCGAGTCCAGCTGTTCTTTGTGTTTCCAGAAAGGCTTTGCTTCTTCTCTTACCCACGGAGTAGAATCCCACTGGGTATCTGCT
GCCCACTCCTAATATCAAGTATGTAGAAGAAACACACCTGATCCAGGGATGGTGTCAGGCCCATGTCACTTTACCTTGTGTCTCAAGTAAGCAC
TGTGAGGGGTAGGACACTGCTGCCCAGATCAATACCTTCTATTTTATTCATTCCATCGAAGGAATCATTTCACACCCTTTGAATCATGGCCCTG
AGCTCCACTACCAACCACTGCACTAAATAGTACCAGATGTTGCTACTGGAAAGACACAACAAGAAAAAAAAAATCAGATGCGACCACGGCCCAG
GAGGTGTGGGGGCGAGGGGGGTGGCAGGAGGGAGGCAGCAGGAGGAGCGCAGTCGTAACCTGCGGATCAGGTGACAGCTGCAGACTCCACCCTC
AGGCAACGGTTCAGTTCCAGACTGCTTTCACTGGTTCCCTCTTGAAACCGAGAGCAGACTTCATGGAGGACAGTGACTGAAGAAAAACGGACCA
TTTTCTTGGGTTTTGTACTTCCATGCTTATAAAGAAATCACTTTTTTTTTAATGCTAAGATTTTGAAAAGAGTGAGTCATACGATTCTCTCAAT
GTGATATTTTAATGCACCATTCCAGTCGTTGACTTCAGGAAAAAAAAAAAAAAAAAAAAGAGCAGGAAAGCCCAGGGGTGGGATGTGTGCTTGT
TGAAGGAGCACCTGGCCCAGTAGCGGAGGACCCAGAAGATGGGCAGACATAAAAACTGATCTTTTGATTTCAAGTCTAAGCACCTTTCAGGCTG
ATCACATTGCTATTTAAAGATGGGCCTTGGGTCTCCTTGATGAGTGCATTGTCCTGGAGGTGCATCCCATTAAATACAACTTCCCTTCCCCCAA
GTCCTTGGCTGTCTTCAAGTGAAAATTCCATTGGTTTTTAACAAGACTGTCCTCCTCTGTCCTTAAAGCTGACPTTCCAAGTCATCTTAACCCC
GGGTTGTGTCTCAGACTCTGGAAACTTTAACGTATGTATAATGTGTCAGAAGTTTTGGGTTTTCTGACTGTTTTGTTTTTAGTTTAATTCAATT
AGAGAAACCTTTGAAAATGGTGATGTTATTTCTGGTATTTAGGAAGCATTTCCGTACTGTCAGTGTCTTGTGTCATCAGAATACCGGTGGCTTA
GAATCAGAGATTCAGGCCTTTGGCCTGGTACACGCTGAACCCCCGCATCTTATGATGGAGCTTGTTGTTAGTATATATTTAAATGTCTCGAACC
TTAATAGTAGAGAGGTAGAGGTGTTATGTATTAATCTATCCCACTCTCCGTAGTGGGAGCTAGATGCAGGAGAAGAAAGGCAGGTGTAAATCTA
GGAAGAGTGAGTCAGCAGAAGATGTGCTTGGTCCCGGTGACTCCATCCATGAGTGCTCACATCTCTCGCTTTTGCAGTCTAGTTAGTGGGGATT
GGGATGTTAATTACACATTTCCCATCCTTCCTGGCTGTCTCGTAGGCCTTCGTTTTGCTTTGAGAAATGGAATCCTCTGAGACAAGCCCACAGA
CCCACCATCGGTCTGTTGCCTGGTTGCTGGGTTTTGACCTCTCCTTTCCTGTTCTGTCTGGCCTGAGATACTGCGAGGGGTTGGAGGGGGTAGG
TAGTTAAGTGACCTCAGGAGCCTGTTATAGGGAGTAGCATGCTCTTAGGCAGGATCAGGTACCCACTTTCACCTATTGGAAATTCGGAAACTCA
AAAGCCTAAGGTATCCCCTCACCCCAAACATCTCACGTCCCTCTCCTCCCTTCTCTCCCCTTCACTCTCCCTCCCCCACTCCAAGTCTTCTTTT
TTCCATTCTCTGTCTTTTTGACAGGGTTTCATGTGGCCCAGGCTGGCCTTGAATTCAGTTACTGAGGATGTCCTTGTGTTTCGACGACGTCCTG
TCTACCGAGGCCCCAGTCATGCACCACCACACCACACCCTCCTCCGCTGTTTGGTCAATCTCTGATTCTCCCTTGCCTTATAACATACTCGTCC
TTCCTATTATAAATAGACTTTTTCTCTTAGAGGAAGTGGACGTTGGTCACTCGGAGACAAGATTCCAGGGCGGATTGATGACTTGGAATTTTGA
TCTTGTCATTTCAACACTTGGACAAGGGAACTTTGCTGTGCCTCAGTTCCCCCAGCTGTGAGAATGGAGATAATGACGATGTTCACGTGGCGTC
CGGGCAAGGCATGGGGCCAAGGGAAGATAAATTAAATAGTTGCTTATTTAAGTACTATAAATCATCTCTCGCCTGTAAACACTGATATTGATTA
AAACTCAACATAATTATAATACAAAATAATTATAGTAATAAGCCATCTACTTCTTCTTCTTTTTTTTTTTTTTAATGAGGATTGATTAAGTAGC
ATTGTTCCATACAGTGATCTGACATCAGTACTAAAGCAATAGGAAAATCTTCGCTGCTTCCAGGATCTTCTTGTTTCAATGTTTAGAAAAGAGT
CAGTATTTCAGTGAGGTGGACAGATGACTACCCACGTCCTTTATTTTTTCPGGATTGGTGCTATGTTGGAAAAAAAAATACATGCAATTCCTCT
CAAAGAAAACTGCAGAAAATTGAGTGCTAGTAAAAGCTGTTAATATACACTTACTGTTAGAACATGAAGAACATTTCACAATGGAAAAAAAAAA
AAGATGTACGAGCCCTGCCCTGCCCACCCTGGTTTCACTTTTACATAACCTCTCCATCCCTTACCCATATATGTGCATGCTTTGCAGAGTCTCA
TATAGAAACTTGAGCTGATTTTCACTTTGTGCTGCAGGGTCGGTCCCTTGCTTCCTGCATGGTTATTACAGATTTTCTTCTTCAATGCTGTCAT
TTGTCTAGTGGCCACATTCTGCTCAACCGACGACCTCCTAATGGACATTACACTTGCACCCAGGATCTTAAACCCGAGCAAGGTGTGGGGGGGT
CCCTGCTTCAGAAACCACTCAGTCTTCCTCTCACCCCCTTTAGAAGAATGAATGCTGGCTGTCAGCCCGTTGGGAGTTATCTTTTCTGTTGTCC
TAATTCACTTTCACTTTTTAGATCCTGCTGCCAATCAAAGTCCTCAGAGTTAAGGGAAGTCCCTGTCCTTGACCCACTGCGCCTTGGCTCTGAC
CTGTGTGTGTGCGTGCTATATATGTCATTCGATACATGGGCTGTQATCCTGGGTTTGTTCTTTGGTTGGGGCACCATCAGGCAGTTTGGAAACT
CCAAGCAGCCAGATATTCTATGGAACTAGAAGATTAGCCAGGGCTACAAACAATCCACTCTCTGAATCACTCACAGTATTACATTGTTGATAAA
AATATATTATTACCTTTGGGGCGTGTGTGTGTGTGATGTGTATGTGATATATATATAATATATAACAGCACCAGTGTGCAGGTCAGAATTCAAC
CTGTCAGACCCTGTTCTCACTTTCCACCATGTGGCTCTCACGATCTCACGTTGCTGAGCTTGGCAGCAAATAACCTTTGCCTTCTGTGATATCT
CACTGGCCTTGATAAAACACACTAAAACATCAGTACCAACAGAAAAAAGCTAGACCCTGTAAGATGCTAAGTATATACATTTATTTCCCCCATG
TCCCATTTTTACGTCCACTAAGGTCCCCAAAATCTTTAAGCAAACTCAGAAACACAGGGCCATGATATAATCAGAATGGGAAGAACTCCCACCA
GTCCTTTCCTGCAAAGGTAAAACACCCTTTAAATAGGAGTTAATTCTCTAGCAGATAACTAGCTTGTCTTCCTTGATAAAGTACATATTGGGTT
GAAATTATTATAATTAATTTTGCATGTCATATAAAAGTCCACTATGCCTGCGCTTATTCCATGGATCTCTTTATTCTTAGCCAAGGGAGAGCTA
GACGCCCAGTCCACTGTGGTGAGCTTTCGTGGAACTAGGCAGATGCCCAGATGATGATGCCTGGCAGGCTCCCCACAAGTAGGCGATGTGACTG
TGAAGTACCACCATCAACGGGCATTGAGCTTTCAGATTGATGAGGGTGTGTGTGTGTGTGTGTGTGTCTGTCTGTCTGTGTGTGTGTGATAAAG
ACCAAAAGCCAACATTGCATGTCTATCTGTACTCTCCTATAGTTTTATACATGTAAGTATGTNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
TTTGACTACTGGGCGGTCTCCCCAGCCCACCTTCCACATCTTCTATAAAACGTTTGGAAAACGTTCTGAGGCCTGAGAACCAGAWGCTCCAAAG
TTGTCCAAATCATGACCTTGACATTCATCTCCGAGGGACCTCTGCTTTCCTGGTTCTCAGTGAACAGCTTTTCCACACTGTGGAGTGGGATTCC
TGGCTGGCTGGGACTGACTCGGCGTGGCTGGTGGCTCTGACATTAACCCTTGAGGTCCATGGACATCATTTTGTGACTCAGCAGGTCCCAGCTC
CTTCCTTCCTTCAGCTTTTCTTCCTTACCTTCCCAGAGCTCTTCAGAGCTCTCAAATCTTTCAGATCTGGCCACATGGTGTCTTCCAGGGGTCT
TTGCCCATCAAAAGCCATTATCCGTGGTGGCTTTGTTTTGTTAAGGATTGCAGTGTGTGCTGATGTGATGTACAAACTTGGGAACAGAACTCTT
GTCAGATAATGGGGTGGAGTGTTCTTTTCTGGAGAAAAGTCGCTATTTCATCTATGGCGACACAGCTCACTTTCTGGTGCATGAAAGCTTGTAG
AACCATCATTTTCTGTTACTGGTTGCTAACTCTQTCCATGTGAACACATGCTTGCACATACAAACACATGCTCCTTTCTGTTAACATGGCCAGA
TAGTAATGACTTTGCCACTCAGCAGGGACTCCTTGAATGGGGACATCACCTTTACCTGAGCTGTTGTCTCCTGGTAGCTATGCCCTGCCTGATT
CTCGTCGCTAGAGCTAGTTGAAGCAGCTCTGAATCATACTTATCAGATTTCAGAAACCCTTCTTGGGTTGCAGTGTGAAAGACTCAGCTGCATG
TTGAAACCATAATTTGGTCAAATATGTTATTAGTGTTAATTTTGGCCTGTTTCCCTTTACATTTCAGGGTAGTGACTAGAAAAAATAGTCTTGC
CATGTAGTATCCTGCTGACATTTCTATCCAGTGAGGCCAATCTTTGTATAATGATAAGTTATCATTTCTTTCTAAGATGAGAGCATACAGGCTG
AGAGTGGTTCACACAATCACTTGGAGTCACACCGAACTCCCCTGAAGTCTAGGCTGACTCCCACCTCCACAACATTTATGGCTATGGACTTGTA
AATTGATTTTTTTTTTCTGCCCTCTGTCCCTGAGTTGTCGGAGATGATCTCTCACCCTCTGGAATCTACAGAGAGATAAGAGTGCCTGAGCTGC
TCCTGCAGAGCCCCTTGGCTCACACCTGAGTTTATGTTAATGAGATGACTCAGGGGACAAGCTGGTCATCTATCAGAAGGGCCAGCCCTGTGAC
TGGCCCGGAAGCTTGGAATGTAGCCAGCCTCCGCAGAAGAGGCAAGCTGGACTGAGGATTAAGTTCAGACACGGGGCTGATGATGAATCAAGTC
CAGTTCCACCTAGTGAAACATGATCCTGAAGAACACAGAAGCCTCGCCCTCGAAGCTCAGGCAGGCATCCTCCTTGCACACAGGGACATGCCAG
CAGGGTAAATAGAGTGTCTTCATTTCACACGACTGAACAGAGAAGTGCACGGTGTGGAATACTAGGGCCTACGTTGTGCAGCTTTCATTTTGCT
GGCCCTGGCTTGCATCCTTTATAGTGAAGCCATTAATTAGCACAAACAAAAGTGCTTTCCTGAGTTCAGCGAATCATCCTAGTGGATTCTTGAA
CCCCGGGTATTATGGGGAAGCAAAGAACTAGTGACAATTTAGTCAGAGGTGTCAGAGGCCTGACAGACACCACACTTACAACTGGCAGTGACGC
CAACGCTGTTGGGGATTGTGCTCGCTTTCTGTAAGAGTCAATCTTGGAGTCGCTGTCAGAGCTCTGTCAGAGCTGCATTGCAGTATTCTGAGAT
GAAAGCACCCAGTGTCTGCTTGAAGCCCCTGGTGGTGAGAGAACACCGTAGCACTTGGTAAGATAGCAAGACTTGTTTCCCTAACCCCATTTCC
TTGGAGACAAGAGGCAGCCTTCACTCTAGTCTTACAAAATGATGTGGTCTTGGGAAGACTGCTTTAGACTGCAAGGCTCCAGGTCAGCAGGGAA
GACCTGTGATATTTCTAGGCCCAAGACCTTCCCACCTTTGCAGGCCCCCTCTATTTCAACTCCTAGGTGTCACCTGAGGGGGTGTGGTGGCCCA
ACATGCTGGCCTCTTTCTTACAAGAAATTCTTGAATATTGATGTTGAGATTTTGTTCCAGTAAGGAAAGCACTGTGTGGGATAACCCTAAGTAA
GGGATTTGCATTACTTGGTCATTTGCATTGTTAGGATTCATACTTGTGGCGTGTCTCACTTTGTTTCAAAAATATGGTGTCATATCCAACAGCG
CCTTTTCTGTGGACACCCCTGTTTGGATATTTTAGCCATGGTCTTGTATCGTTATTTTTGGTCTATTTTGTTTTGCTTTCTCCTTCCTGGCTTA
AAATAGACAAGGTCCCCCCACCCCCACCCCCACCTCCAAGGCCATTAGTTTTTAGCTTTGCTCAGACTAGTCCAGTCCAGCAAAGCCTTCTTAG
GTCACCAACCTCCTAGAAAGTTTGACTCCTCAAGGCACGTGATAGGGCCACCCTCCAAAATGGGTTTCTAAGAAAAGAAAACTGTCATTACTTC
GTGGGAGGGGTTCTACAAGAAACGGGAATGAAGCAAACAGTATGAAGAGTTGTGAAATCTCCTGAAATCTGTTTAACCTGGTCTGGCCCTGGGC
TCTCTGCACTCTGTCACCAACGCAGGCTTGCTTATTAGAAGTTTAATGGTGGCGATAGTCCAGCCAAATAACTGACAGTAGCTTTCACTGAGGC
TGTCAAGCAGCTCCCTGCCCCCTCAGTAAGGGTCAGCATCCAATGTCAGCCTCAGTTTCTAAAACAGATAGAACAGATGCTTAGAACTGACTGC
CCTAGAGTTGGGTGCTCCCTTCCCCTGAGGTACTTCCAAATGTTTGCCTGTCGACTACAGGGTTCAGAGTACACCTTTCAAGACATAAGTATGT
GCTACAGCCCTCTGAGAGAGCGAAGGATGTAAGAATCCCCTTCAGGAGAGAGGAAACTCTCCCCATTTAAGTCAAAACTATTAGGGGATAATCC
CGGTGTCTTATTTACTTTTATTTTTGTGTGTATGTGTGTCTCTATATATGTCCACTTGTACATGCCTCCTCTTGGCTGTGTGTGTGTGTGTGTG
TGTGTGTGTGTGTGTGTGTTCCTGTGCTTACCTGGTATATATCCAGCGAGCCCCTTGGATTCTCCTGTTTCTGCCTCCCAAGGATGGCTGGATT
CTTCTGTGGGCATTCGATCTCAACTCACACTCTCATACTTACCCACTGAGTAAGCATTTTACCCATTCAGCTGTCTCTCCACTTCTTTGATAAT
GTGAGTTTAAGTGACCTGAAATGTAAATAACTAGCCTGCCAGTTCAGAGTTAATAAGACAAATAAGAGGGACTCGAGAATGCCTGATTGTAGAG
CTGGTATTGTGGGAGAAACAGAGCCACTGGGTCAGTCAACACCCTGTCCAAAGTATCTGTGATTGTTCATCCTATTTAGATGGAAGGGCTGCTG
CCAGGTCTAAGGGCCAAACCAGACCTTTCTTAGGCATATGCCTGGGACAGAGCTCTCTATCCCACTGTGAGCACTTCTTGAATGCATGCTGACC
TGAATTCAGGCAGGGCCTGTTTCCAGACCTCTCTCCGCCTGGGTGTCCCTCTTGACCATTTTTCCTCACAACTCTTTTTCATTCACTTCTGTCA
ACATTGCTCATAAGGGAAGTACAGAGATGGGAGCATGTGAGAGGAGGGACAGGCTAGAGGCCGCCAAATGTGACAGATCTTATTCTCAACCTCA
CCTATGGCCCTATTTGTCACATGTAAGGTCCAGCAACTGTTCTTTGCCTCTTCCTCAGCCTIGTACCAGCAGAGTTCATCTTCCCTGCACCAGC
TTCCATGAGCTGTGACCCGTGGTGTCATCCAGGCGAGTCTCCCAGTGTTCTCAGAACATTCTGGCATTGATACTTCGCTACAGTTTTTTCTAAT
GTTTCCTCGTCCTCATCCCTTCTTTTTCTCTCCAATTAGATTTATCTTTGGGCACTATTTTTAATCTCCCAGATCCCTACCATAATGCGGAACA
ACACATAATTAATGGCTCTTTCTGGCTCATTCTGATGTTCATCTCATCTAAAAATATTTAACGAGCACTTGCTATTCAGAGCAGACGGTACAGC
CTGAATGAATACAATTTCTCTGAATAACTCATTTAGTAGTGGTTCATTTAATTATTGTATCAACTCTGCTGCCCATTGATGCTCTACTTTACCC
AGAAGTAAACTAGGATCTAAAGATGCCAAACAACCTTCCGGAGCCTGTACCGCAGAGGCGCAGGGCAGCCTGCCACCATTTCCCTCAGCAGACA
TGCCTTGCCACCTAATAGCACCTTTGCTTTCCTGGGCTCCACTTTTTTTTTTTTTTAATATTTTTTATTAGGTGTTTTCCTCATTTACATTTCA
ATGGCTATCCCCAAAAATCCCCCATACCCTCCCCCCCCTCCCTTAACCACCCACTCCCACTTTTTGACCCTGGCGTTCCCCTGTCCTGGGGCAT
ATAAAGTTTGCAAGTCCAATGGGCCTCTCTTTCCAGTGATGGCCGACTAGGCCATCTTTTGATACATATGCAGCTAGAGTCAAGAGCTCTGGGG
TACTGGGTAGTTCATAATGTTGTTCCACCTATAGGGTTGCAGATCCCTTTAGCTCCTTGGGTACTTTCTCTAGCTCCATTGGGGGCCCTGTGAT
CCATCCAATAQCTGACTGTGAGCATCCACTTCTGTGTTTGCTAGGCCCCGGCATAGTCTCACAAGAGTCAGCTATATCTAGGTCCTTTCAGCAA
AATCTTGCTAGTGTATGCAATGGTGTCAGCGTTTGGAAGCTGATTATGGAATGGATCCCTGGATATGGTCCATCCTTTTGTCACAGCTCCATAC
TTTGTCTCTGTAACTCCTTCCATGGGTGTTTTGTTCCCATTTCTAAGGAGGGGCAAAGTGGTCTTCGTTCTTCTTGAGTTTCATGCGTTTAGCA
AATTGTATCTTATATCTTGGGTATCCTAAGTTTCTGGGCTAATATCCACTTATCAGTGAGTACATATTGTGTGAGTTCCTTTGTGATTGGGTTA
CCTCACTCAGGATGATGCCCTCCAGGTCCATCCATTTGGCTAGGAATTTCATAAATTCATTCTTTTTAATAGCTGAGTAGTACTCCATTGTGTA
AATGTACCACATCTTCTGTATCCATTCCTCTGTTCAGGGGCATCTGGGTTCTTTCCAGCTTCTGGCTATTATAAATAAGGCTGCTATGAACATA
GTGGAGCATGTGTCCTTCTTACTGGTTGGAACATCTTCTGGATATATGCCCAGAAGAGGTATTGCGGGATCCTCTGGTAGTACTATGTCCAATT
TTCTGAGGAACCGTCAGACTGATTTCCAGAGTGGTTGTACAAGCTTGCAATCCCACCAACAATGGAGGAGTGTTCCTCTTTTTCCACATCCTCT
GGACTCCATTTTTTAGCAATAATAAAAAACTAAGGATGACATTGATGATTTCGGATAGTGGCAGATGTTCTCATATGTGGTCAAGGAGTTAGCT
GAGAGAATACTCATAATATCAGGAAGAGCCCTTCTGGGAGACCACAGTCACTGAGCCCTGCGTGCTGTCTGCTCTGCAATGGTTAGGATAGGAT
GGAATGTCGCCCTCAAGTGTCTATGTTTTGCAAACGATGTTTTCCCGACATGGTGATACTGCAAAGTTTGATACCTCATAGGAGGTCAACTTGT
CACTGTCCCTGGTAGAGCATGAGACTGGTTTTGTGGAAACCTGACAGCAGAGTTGTTATTTAAAAAAAAAATGATGAGAAAAGAAGTGGGTAGT
CCAGCCCAGCTGTTACCCTGGTTTTCTGTCCCCCCTATTTCCCATCCCAGCTGTGATGCCACCTGTCATGATGTGACACAGCCAAGGTGACCTT
CACCAAAGTTAGCACCATACTGTAAGAGCTTTAGTCTCTGAAACTAGGAAGTAAACAAATCTGTCTTTGTATCCCGCCTCAATTACTTTGTAAT
GGGAACGGAAACAGATTCCTGCACCCTCCCCATATAGAATAATCCTGTGGAGGTTTCTTCTACTCCTTGAATAGTTCCTCAGCTGACAGCAGCA
GCATGTCCTAACATTTCACTTGGAAATCATGTAAGATTCTCCCAGGCCAAAGGCTCAAATGCAGCAGAACTCCCAACTCCTGGTACTGGTTGCA
AGTCCCAGGGTGTCACTGCACTTCTGACCAACTAGCTGGCAATAAAACTTCTAGAACTCATTCTCTAGATTTGATTAATTTGCCATGACAGCTC
ACAGAAATTGGAGAAACATTTAGTGAATTTGCTTAATATAAAGGATATTATAAAGCGTGCACATGAACAGGCAGACAGCAGAGCCATGTAAGAC
GATGTGCAGGTCTTGTGTCATATGTAGGCATGCTATCCCTCCAGTCACTTCCACATGGCCCAGCCATCTGGAAACTCACCAAACCTTGTTCTTT
TGAGTTTCTATGGTGTTTTCATTAAGTCTGAATGATTGATGACATTTTTGGCCATAGGTGAGCAATTCACTTTCATCTTCTCTAGATCAAGATT
GACCGCTAGAGCCGAAATTCCCTAGCCTCTAACCCTGCTTTCTACTTTCTGGTATCACTTCCATCTGGAAGGTGTTAAGGGCTCCGAGACTGCC
ACTCACCTTGTTGGCATACAAACAGTCACTCTTATCTCTTTGGAGATTGCCACGGTCATCTGGGTGTGCGTTGCTTAGTATCACAGTATCACAC
TGAGGGAATGACTTGAAAGAGTGGTTAAACACAGAGAGCCTTGCAATAGATCATTGATGTCTCACACCTTACCAACGGACTGTTCTTGCTGTGA
CTCACCTCTGTCTGCTTTGTGTTGTAGTAGGGCTTTGTTGAGCTTAAGGAGTGCTCGAGGGGTGGGGTCAGGAGGGAAGAATGAAAGAATTAAC
CAATGAGCTTGCATCCAAGAGGTTGGCAATAAATGTCATAAGCAAGAAGCCGAGTCCCTTTAGTGTGGAAAGAAGAGCTACTCTGTGATTCCCT
GCCCTCAGGAGGTCTTCCCCCCGCTGTACCATGTTGGATATGGCCTTCAGGGAATGTGCCTAGCAAGTCCGACGGTGCCAGAGGTGGAGTGCAA
ACAGGGTCAGGTTTCTGTTCTTCCCTCCTGGGCTGCAGGGATGGTTACTGGGATGGTTCTCCAGTAACCTTGAGCTTATGTTTATAATCTATGA
TCCCACTCCAGACCAGCAGGCTGIATTGGTTGGACCATAATCTGGTCTACCAGATGGGGACACCTCAGCTTTTGACCCGTTACTATATCCTGGC
ACTACAACAACCACCTCCAGAGGTTTCCAGACCCTTTCTCCTATCTCTTCCTGCGAATGCAGACCATGCCTGGTTTCAQTTTGACCTTGCAQAG
CTCAGCCATTGCGGGTGATTTGACTTTGGCATGTGGCTGCAGAGGCAGCTCCTGCCTTCCCTTCCAGGCAGTTGATGGTTAATATACTTATCGT
TGCTAACTATTCTTGCCAAATATAGACTTATAGCTCTGACTTACAGAAGTCAGAAGTAGAAGCTGTTGCGATATATTTTTTGCGGACTGAAAAC
ATTGGCTAAAGAAAGCATTCCAAATTGTGATAGGAGCAGTGGCGCTTCTCCCATCTCGCTTCCTCAATTCCCCACGCCAAAGCCCTTTGACCTG
CAGATCATTACAAAATCCATGGCTGTAAACGCTGTTGTTTGAGTGTGGGGTTTCCTTCAAAACAAGTTGAGTGGCTTACGGATTTGACTGGCCA
GTTCTGTTCATCACCATTAGGCATGGCTCCCAACCCCAGTCTCCATTGTAACCCTCGTCTTTTCCTCCCCTCTCCTGACCATAGATGACTTTTT
TGTTTGTTTGTTTGTTTTTTTGTTTTTTTTTTTTTGTTTTTGAGACAGGGTTTTTCTGTATAGCCCTGGCTGTCCTGGAACTCACTTTGTAGAC
CAGAGTGGCCTCGAACTCAGAAATCCGACTGCCTCTGCCTTCCAACTGCTCCGATTAAAGGTGTCACCACCGCCCGGCCCATAGATGACTTCTC
ATGACTTTCTCTGAATATAATTTCCAGTGGGAATGCTTCATGGTAGAAGAATGTAGTCTTTTGAAGCTTGCCCTTGATGTTTCTAGAGATTTAT
TGGATTTTACTTAAATCCCATTQGGAAACATATTTTCGCTGTGAAGTTGTGATGTGTTTAATTTGGGGTATAAGATAAATATGGTCCTGGGGTG
GAGGGATACATGGATAGTGAAGTAAGTTTTTCTTTTGTGCCATGCGCTGGTTTAATGAGTAGCTTTATCTGTTTGCTTAGCTATGTTTGATTAT
TTAGGCTTCTTCTGCCTCAAAAGTAAAGAATATTTGCCCTGCCTTCAGAGAATTAAATGAGCAGCATAGAGTATGTGGCTCACTCTTGGGAAAA
ATATGACTTTGTTTAATAGCACTACACTATTAACATTCTTCCTCTTCAGCTTTCCACTGCCATCTCAGTGTGTCCCCCCATCCCTTCCCCTCTT
GATTAACCACAGAATGTAGGGTTTCCCAAACACACAAAAGGTCTCCAAGGAAAACAGTTCTTGGCTCAGAAAGATACCTCAGGGTGAGCTTGAG
CCAGGTCTTGTCCATAATGAGGTTGAGGGATTCTTCTGAGAAAGGATAACTTTTGTGAGTGCTGCCGGCGAAGAGAAGGTTCTACATGCTTGCT
TCTTATGGCTTGCCTGGTGGGTTAGGCAGCCTCCCACCCCCACTACCATCTCAACCTTGGACCATATCTGGCTTGACTATAAGGTAATGATATC
CTTAGGTTGGCTCTCAGGAGCCTTGTCTTAGGTAGCCCACAAGTTCTGCCATGACTCTTGAATAGCAGATATTCTAATATACAGACACTTATAT
GGAGAGAGTTAAACTGAGAAATAAAATGTAAAGTGGCTCTATATTAGCTTGCTTGGGTTACGTATTAGTCAGTTTTCTGTTGTGATAACAAAAG
TCAGTGCTGGATACTTTATAAAGAAAATATTGGTTGCTTTCGTGGTTTTACAAGTCAGGAGTGTGAGATCCAGTGCCTCTCTCTGTTGGGCCTC
TGGTGAGGGCTTTGCCATGGATTACGTGGATAATATTTCAGCTGTGGAAGTATGTGCCAAAGACATCAGATGTCAAGAGTGGAATCCAGAGACA
CTCAGGGCCTAGACCTGCTCTCATTGCTACTCACATGGGTCATATAAGAAATGTGTTCACCCCTTCCAAGCCCAGCACCTGTTGACCTTACGAT
CTCCCACTATGCCCCACCTGTTAAAGGTTCTAACATCTGTCAACATCACCACCCTACAAGCCTCTTAGTATTTGAACTTTTGATTCCTTGCTCA
AAGAACATCCAAACCACAGCACGTTGCTATAACAAAACACCCCAGGCTGGGTAACTTAAGCAACAGGAACTAGCTCTCCCACAGCTAAAGGGCC
CAAACTGTAAGACTTGTAAGATTGGAGTAGCCAGAACGTAACTGTCCTTTTACCTTCTCTCACCCGCTCCTTCACCCAACCTATTTCTTGTTGT
TTGTGCGGATGGCAGGCACGCACATGTCACIATATAATGTCCATGTCAGAATACCGGAAAGGGGTGAAGCAGGAGGCAGAAGCCAGCTGCAGGT
GCATGGGAAGGAGCCCGGGGCTGCTCCCCCTTGGAATCACACTGATTTGTGCAAAGCCTAAGCCGGTTCCTGGCACGGGACTCAAAGATCTTAC
AGTTTGAATCCGGTTCACATAAAAGTCATAAGCACGAGAAAGCAGGCCCACAGCCCTTTAATTGTCATCGTTGTGGAAAGTTACATGACTGTCA
TCACCTGTTGCTTCTGGCAGTGCTTTCTGGACTAATGTAAATAAATGAGCTAACAGGTTTAAAGAAGCAGCCTGTATTTACATACTTGTGAAGT
AGCCTCAAACAATCAGTCTGTGCTATTCAAACACACACACAAACACACACACACACACACACACACGCACGCACACACACGCACACACACACAC
ACACACACACACACACACACACACACACACACACACACACACACGAAATGTGTGGCTTCCTCAATGCAGGCTCCTCATGAAGGCCTGAAGTCTG
AAGGTTATGATGAGTAGTGTGAGTAGTGGAAGCCCTGCTTTTCCCCTTCATGGGGTTTTGACCTGAGTCTGCCTTTTTGATGAGAACAGGCTCT
CCTCAGCAGGCTGAAGTTTAATCTTCAGCCATTTCTATAGCACTTCTTTAAGGCATGGACTTGGGAGAGCCCCGGACTGCTTCAGAGGAGGGGA
GAGTGAAGTGGCAGGGGCAGAAAGGAATCCCACACCCTCTGATAAAACACTCCAGAGTCACCATCTCTTGCAAGTCTTCCAAAGGAAGCTAGAT
TTTCATTTTTTTTAAAAAAATCATAAGGTCCTTTTGACTCAGTTTGACATTTTGTAGATAAATATTTAGGCAAGCATATTTTGGGTGTGTGTGT
ATATCTAGCTCTGTATCTGTGTGTCTCTGAAGGTCAAAGGTTGACATTTTGAACATTTTGTGTCTGATTCAATTACTCCACATTACTGAGACAG
GGTCTCTTGCTGAACCTGACGCTCACCAACTTAGCTCTCTGTTTGCTAGCCAGGGAACTCTGGGGACCCTCCTGTCTCTGTCTGCCCAGTGCTG
GGACTTGGGGCATCCAGCCATGTTTCTTACATGGGTCCTAGGGAATCCAAACTCAGGCCCTCATGCTTGCAGAGTAAGGACTGTGTAGTGACCA
AGCCATCACTCAACACCCCAGGCATGATTGTTTAAAAGCACATTCCTCTGGAGGGTACTAATGATGCTATCAAAGCAAACACCTCAAGAATCAT
GAACAAAACATAAGCAACTGGCCCTCTAACTGCTCGTGGATGAGTCCATTGCACCACAGATTTAGAAAGAAGCCTCTTACTAAGGGCACAAGGA
AATCAGGAGGGAGCAGTTACTGGTTTGGAGTCTCTCAGTCCCTTCATAGTCTGGGATAGCTTCTTTCCAGAAGGCTAGGTTTTGTTTGTTTTTC
AACACACAGCATGATTCAGGCAGAGGAGTGGTACGCACCAGGAGCGAAGCAGAAATACCCATGCTTAGAAGATTGTGCAGAGGTGTCTACTTCC
TGTAAGCATCTATGGCCTGTCATATTCTTTTGTCCAACACAAGAATCCAAAAAAAAGATTCAATGAGTTCCAAATTCAAGCAAATTCTGGTATA
GCTTTACCAGATTTTTCTCCCTTTACCTCTCTAAATGTTATGAAATCCAACTGAGTCAGATTCATGAACACAAACAAGAGAAATGACCTTTACA
CCGAGGTCCAAAGATAGTTTGTGATGGGTTTTGTTTTTCTTTCTGAAATAGGATTTCATGTAGCTTAGGCTGGCCTAGACCTTTCTATATAACT
GAGGCTGGCTTTGAAGTCTTGATCCTCCTACCTCCAGGCTCCCCATTGTGAGGGCAAATATTAGGTAAACATGCCTGGCCATACATGGCATTTT
TGAAGTGGCCTCTACCTAGGATTTGTCACTAAAGTAGACAGGGAGAGAGAGGTTTACTTTATGTTTTAGTGTTCACAAATGGTTTTCATAAACT
CAAACCATAGTTCCCTACTTCTTCTGTGGCTCCTGTGGTACCATCTCACCAGGTTCCATGCCCCCTGTGGGAGACCCATGTGCAAGGGGACTTA
AGAGTCCCTCTCCCCAAGAACAGATCTTCCACCAAAGCTATTAACCTGGAAATATAAACATGAACTGATAAATTCAAAACTAGAATATTCTAGC
ATTGTTTTAGTTCAAAGTTACACACAGGCATGCATCTGGTTGCTAAAACTTTTACAGAGTAGTTCTGAGAAAAATTGGTAGTCATCATCAACTA
TTGAGGCATATACTACATAATAAATACTTTQTATATGTAAATAGTCTATTTTTGTTTACCCTTTCTATAGATTACCATTTCCTTAATGAACTCA
ATTTATCACAGAAGTTTTTAATTGTTGGTGTGTGTGTGTCTGTGTCTGTGAAGTTCCGTTTCCTAATTTCCTGTGTTTGTTTGTGACAGAGTCT
TATGTGGCTCAGGCAGAACTTGAACTCACTCTGTAGCAAAGAAPAGCTTCGAACTTCTGATCTTCCTGCTTCCACCTTTCAAGTATTGGGATTG
TAAGTATATGCCACTATACCCAGTTTATGTGCTGTCGGGTATTGAACCCAGGGCAGTCTGACTTCTAGGTAAGCCCTCTAGCACCTCAGCCATC
TCCCTACCCCATCATAATCTTATTTCTGGAATTTCTAAGACTATATATCATATCCTTACATATCATTTCTACAAATACACACATACTTCTTTCA
TATAGTCTGAGCAAGTTTGTCAGGATTGCACTTTTTTTGTTGCTTGCCAATCTGAGCTGTCTTCCCCTTTCCTTGGTTTCTCGGAAACAAACAG
TCAGACAGAAAGGGCTAGCTGGTGAGAGAGGGAGGGAGGGAGGGAGGGANNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNTTCTTTACCTAGTACTCACAAAACCACTGGGACTCAGCCCCTTGCCCCTCCTTTCCTTTGCCTTGGCCTTTCCTCCATCCCTTATGTG
CTAGAAGACAGCACCTTCGTCCCTCTTCTCTCTTGCCAATATAGCTTCCCACAGGGCTGGATCCTAAGATAATTTAATCCATGCCTCCTTGTAC
TAAACTTGAAGTCACTTCTACTGTCAAATGCCCACTCTCATCTATGGGGTATGTGGATAAGTTCATAGGAAATGAAATGTAGAAGCCCCCTTTG
ATGGCTGTAGCGGCTGTTCCATGTACTTCTCATATTAAGGTGCTCTGTCCCCATCTCCGGACATTTTGGCCTTAGACACTTGACATCAGCTAGG
TCTTATTCTTTCGCCTTATCTTTGTGCCTACAGAACCTCAGGATACTGCTTGATATAGTATCATTTCCACTGGGGGAGCTATAAAATAATATTT
ATTATATGCCAATAATTCTGTCGTTTGTGAGCTATATTATATAGAACTGTGTCTGTGTGGAAGTCTGTGGATGAATATGTCCAAGTTACTGATA
TGGCAACTGACATGCGCGTATACTAAATAACCCACTGGAGGTGATGCAGATGGTCACCGCTAGCACCAGACCGCCTTTTACTACAGACTTGCTC
CATCCAGTCCCTATATTCACTGCTGCCAATGCCCTGCCTGCCTAGTAACCCAGGAGGAAAGCAAGGCTGTCACTAGTCGTCATCAGCATTCTTG
CAGCAAGCAGTAAGAGTGCCCTTTTAGAGTAGGCAACACCCCCACCCTCCTGTAGATACTAATTCTATCTGCTGACAGTGTTCTCCTTACACTT
CTAAGGCCACTTCTGAGCTTAGCATGAAGCCTGTGTCTGCAAGGTTCCTCTGTAAAACACCGGCGTTTCCTGCTTATGGGTTAGGCATGTGAAT
GTTCGTTCTTTAGCCCCACGGCCAGAGAATCAGATCCCAGAAGGCACTGCGGCAGCTAGCAAGGACTGGGGCGGAAGTGTGGCTACAGACAGGG
GTTTCTCCTGAGAAAGTCTGGCACTTCCAGAGGTTGCCATTGAACTCATTTGGTCTCTGCAGATGGCTATTAAACTGTGAAAATTCTAGAATGT
TCACTTGCCAAAGTTTTTGTATGTCTAAGGCTTTTCTTTTCTGGTTACTAATAAAGCGTTCATCATGTACCCCATATGGTTTCAGCAAACTTTA
TGTTTGTTAACTTATTTTGTTCTTAGAATAGTATTGCATAGGCACTATTTTCTACTCATCGGTAAGGGGAAACCAAGGAAGAGACGCATGAGAA
GCCTGCTTAGGATCTCATGCCAAGGGTTTATCAGGACCGGCTCAGCCTCCAGCAGCTTCTCCATTACTGTAAGGAGTGTAGGTGGCGTCATGGT
GCTCATGAGGAGTATAATGGGGTGGGATGTATCGCATGCCATGTGATGTATCTGTAAACCTTCATGTAGGTGTCCTCTACCCCAGCCCTGTGCA
TAGGGTGTATTATGAATGATTATGTATTATGTAAGACAGTAAACGGAGCTTTCCTAAGGGAGAGGGATTCAACAAGTTCCTCGGAGGCTGAGAT
CTCACTGGAGGCTGGGCCCATGTGTCTCCACCCATCATTCCTCTTCCTCTACTCCAACCACGCCAACAATGTGTTAATGATGCTTTTATTGGGG
TGTGTGTGTGTGTGTGTGTGTGTGTGTGTGTTTTGTTTATTTGGCTACTCTGGCTGGCCAGTGCGGTACCAGCAATCTCCCCATCTCTTCCTCC
CCAGTGCACATATCACACACATATACTATTCTCCCTTGCTTTCTCAGGGACACTGGGTATTCAAACCCTCATCCTCATGCTTGTGAAGTGGGCC
CTTTACTGACTAAGCCATCTTCCCAGTTCCCACCCCCGTGTAGTATTCTTCTGTTATCAGGCCAAACAAAATTACCCCCTCCCCTCATTAGGTA
ATCATTTGATATGGTCTGCAGGGCCAAAAATGACCTCATGAGCTCTACTCTTACCTTCTGCTGCTCCCAATCCCATACTCACCCCTAAGAGGTA
CAAACAACCCCTACGCAGATCCCTCTGAATATCCCTGTCAGATACTGCTAATATCACCCAGTCCTCCGACTCCACCCATAGAGTCTCCCTTTCA
TCGTGACACTGTACAGCACTCTTGTGTCAGGACGGACGCCTGACATGAGCAGACAGAGCTGGATGCTGTCTCTTCTGACCCTCACTGAGTGCTT
CCTTGTCCAGCTGAGATTTCAGTCACATGCGGCCTTTCTCTTTCCCTCCCTACAGATAGTAGTCCACAAGGGTCTTTCCCATGTGGCTCAGACA
TATAATAACACACATCATTTCACATAGATAGGCACACAGCACCCCATGGTAGAAGCGTTCGGTCTGTCAGTCTCGGACTCTCTCTGATCCACCC
TTCCCCCATGTTTTTTCCCCCCACTAGGCAAATGGAAGGCCCCTTGCCACCTGTTAGCATTACGTCATTGGCTCCCCAGTGCACAGTTGTACCC
AAGGCCTTAAACAAAGTAGTTCTTCTTGTATTGTTTGCATTCAAAGAGCTGATATCATGCCAGCTGATGTCATTGTGTGTCTTTGTGTGAGTGC
TATGGCAACTGGGCAGGTGGGGAGCCTCCGGCTGATGTCATTGGGGAAGGAGGGGCAGAGACTGAATTTTAAAAAGCTCTCTGCTGAGTTGTTT
TGTGTCTGGGGGTGGGAGGGTGTTTTGGTATGCCTTTATTTTTTATTTTTTCAAACTAGCTGCTTTAAGACAAATTACAGCTCGAACACACTAA
ATAAGAACAGAGACGCCTCTTCCCCCTTCCAGCCTCTTTTGAGTTTAGCTTCCCAGTCCTTCCTCCCATCTTACATGGAAAAAAAAAGGTTGCA
AGTGCCTTTTTCCTTCAGAAACAAGGGAATTCGGTTCCCCTGGCAGGATCAGCATGCGGCTGTTTACTGGGGGCTTTGTGGAGCCCAGGAGATT
GCCACACAGCTCCTCCCTGTGTCCTCTGTGGAGTTTGCCACAGTGGCAGTGGGTAGTTTTGCACTCTAGTTGTTAAGTGTTCACTGGTGACGTG
GTTTTCCTCGTGTCTTGCATTTTTCTTTGTAGTTGGAAGGCTTCCTTTGGGCTAGCCACCCGGCTATAATGATTTCACCACAGAAGACAACAGC
AAGGGCAGAGTGTAATTACAGAAATGAATTCAAATGAAGTCAAAGCTCTGACATGCTCTCCTAAAGACATGTTTGTCCCTTCCCGTATCTCCTT
TTTGACTGGTAGACTAGACACAGGATGGCCCTCCCTACCAAGTGGACTGTGAATTGTTGCTATTCCCTTCTGAAAATATGATCTGTGCTCTGTT
ATACAGCTGGCTGTGTGCATCGGTTCGGGGACACACTGTGTTCAGGTCACCTGCTCTAGTGGTCAAAAATGAGACTCGTGGTTTGAAAGGATGT
GGTTTCAGTTCAGAAGCTGGTGGACAAGTGAACTTCCTGTCCTGGTAGCAGAGCCTCACTCACTCTGTTCAGCCTTTGTGCTTTTAGCCTTAGC
CTTTGGGACACTCAAGACCTCTGTTACTGGGAAGACATCGCTTCTAGGAACTTCCCAGGGTCTCACCCTTTTCTAGAAGACCCCACGTGTCCCA
CACTCCTTTGGTGACCCCTTCCCAGGTTTCCTAATGTCTCTCTCATATACGTAGTAGAATTTGAGCTCCACAATCCTTCAGGTTTGTTTGTGTA
TAAATATGTGTAGCATAAGTAACAAACTCTGACCCGTGCATTCCCTTAACCGTTCTTTTTTGTATGGCTAGGCATTTTTTTTCCAGAAAGTTTT
AATTAGCTCTTTTTCCTTTAGCATTAAATAGAATCATTTTCTTTGATTTTATAGTTATTCCTTTAGTGGGTACATAGACATCCCATGGATGTAT
TTAATAGTTTTATTTTTGGTATTTACTCAGCCTTTTCTTTCTTTTTAAAAAACTTACTGTTGTTGAGTGTTTGTTTGAGGTGATTGTGTTTGTG
TATGTGTGTGAGTGTGGAAGCCAGAGGCTAGCTTTGTCCAGCCAGCTCTCAGCTTCCACCATTAGACAGATGGAGTTGCGCGAACACCCCACGT
AGCTGACTGCACAGCTGTGCTTGTGCGAGTTCCTTGAGTGACTCTGTACTTATACAAGGCCATGTCTGCATGCTLAATGTCGGAAAACTGGTGC
GGCTTTAACCTCAAACCTTTAAGAGTTCCAAACTCAAGCCTTGAAGTTAGACCGGAGCCTTGAGCTCGGAGTGTGCCTTGTTAGATCCATCAAA
GGTCACTGTTAATGGGTTAGTGTACCCACAATCCTCTAAAAGCTAAGTACAGGAAAATGGGCCACTCTCAGATGCCACTCTCTTGGAAGTCAAG
ATCTACAAATGTCAAGCACATGTATGGAAGAAGTGAAAATGTAGATCCTGAGTCACTCTTCCAACCCTACGCTACATTAGCCAAGCCGCCCTCT
GCGTTTTCTACCTTTGGGCAGTCGCAGAAATAAGATGAATACAAATAGCTTGTTCTTGCATAGTACCCATGCAAGGCTGGGGGTTGGGAATATA
CTAAACACTTTATCTGTGTTTATCTCCATCTCGGACTCACACTCAGAGAGACATGCTTGTGCAACTCAGGTCAGTAGTGCAAGCCGAACACTAG
CCTGTGTAACCTGGCTTTTCCACAAGCCCTGCTAGTAACCGCTCTGCTACTGAGTCGACTTGTCCTTCTGTAAAGTCAGTCATAGAGGGGACAT
GACATGTGTGCACAGTGCCAGGAGACAAACGGAAGTCCTCTGCAGGGCGATATCATCCCTGATTTTCTCAGCTCCCAAAGTACGAGTTCTATAT
AGGCCAGGCACCATATGTCTGTTAGAGGCAAATCAGATTTTCAGTTGATGAAATTACCCGGTGCTGTGTGGAACTTGTTCCCAGGCACCTTGGC
CCAGTTTTGCAATAGGGGTCACAGTGTCCTCTGAAGCTGGCTCTGGGTATCCAAACAGGCTTCTGGCTCTTGTGTTTTATTTTAGAAATGTTAA
AGTACGCACATAGCCATAGTGACACCCATTTCCATGGGAAAGCCGACATTTTTTAGCCCTTCCCCTCTCCTCCCCGACTGTGGGCTTCACTCAT
ATGTGATTTCTGTGTTTTCCTTCTAGGGGGACTCAGTAGTTTTAAACAGAACCATGGAAACCTCTGTGACAACTCCCTCCAGCTCCAAGAGTGC
CGGGAGGTGGGGGGTGGTGCATCTGCGGCCTCGAGCATGCTACCTCAGTCTGTCCCCACCACCCCTGACATCGAGAACGCAGAGCTAACGCCCA
TCCTGCCCTTCCTGTTCCTCGGC2ATGAGCAGGATGCTCAGGACCTAGACACCATGCAGAGGCTCAACATCGGCTATGTCATCAACGTCACCAC
GCACCTTCCTCTGTACCATTATGAGAAAGGCCTCTTCAACTACAAGAGGCTGCCAGCCACAGACAGCAACAAACAGAACCTGCGGCAGTACTTT
GAAGAGGCCTTCGAGTTCATCGGTAACTATCTCTCACAGCAGGGCGGGCGGAGAGCTTCCTCTTGGCTCTCTCTTCCCTCTACTTCTGGTTTTG
ATATAAGGTCTGAAGTGTATTCCTATGTTAGGGAAAACAACAACAACAATAACAACAACAACAACAAAACCTACCTAATGCATAGTCCTTCCTG
TTAGTCTCAGAACTACCTGGATGGGCATAATTGAGATAATAAGCTTTCTGTTGCCATCATTGAAACACCTGAAAGGTTGTCTTAAACGGAGGAA
TTATCTTGACTTATGATCTTGGACGTTTCATGTCTTGGTGCTTTGATTCTTGGCCTGAGGTGAGACAGAACATGGAAGGGAAGGAGTCAAGGTC
AGCTGGAAAGTTTCCTATACTCAGCTTCTCATCAGAGATGCCACTGTCATCAGTGAGGCCTGTAGAGTACTGGTCTACCTCATCGCCCTCGGCA
GCAGTGTTTCACTCCTCACTTCCACATACCCTGACCCTCCATGGCCATCATCTGGAAGGCACGGTTCCCAAGTAGCTCAGAATAGAGAGCACCA
GAAAGTGTTGAGTTTCATATTAAAAGCTTTGGATGCCTGTGAGCACTGGACAACAGTTTTTGTGACATTTCTAGATTAAAACACATCCCCCAGG
AAGTGTCCCCAGAGGAATGCACGTCAATGATAAGGTAGAGGAGAAGGGAGTAAAGAGACTTGTCCCTTAGCTTTAATCACCCACACTGGCTGCT
TCCCAGTAGACAAAGAAACAAACAGGATGTAACTATCTCTCTCACCAGTCTGTAGACAAGCTTGCTTTGAGAGACCCAAGAGTAATGCTTCAGG
AAAGTCTCCCTTTGTGAACACTAAATGACCTCCCACACCAAGGCCGTAGGACATGGTGGATAAGAATGAGTGAGGTGCCCACTGCCATGTTCTG
TGTCACCCCATGAAGGCTACTGACTTTTTGGCAAATAGGAAGGGAGGGACCGGGACAGCATTTAGCCTTGAGTGGCACAGTGTCTGACCTACTT
CCTCAGGCAAGCCTCTACCTTCTAACATTTTAGAAACCTTTGGAAACAGTACCACCAGCTGGGGATGAAGCATTTGACACATGAGTCCGTGGGA
CCATTTCACATTCATATTAAAGCAATAATCATCCTCTTTGTCCAACATCTGTCAACTGGAAGTCCCTATCCTCCATCCTCCCGTTAACAAAGAC
GCCATGGCGGAGGTGGCACTTAGACACTGTATGACCAGAAAGATTAAAAGGGAGAGTGCAGAACTGCTCAGAGCTATTCCTGCTGCCTAGTCTA
TGTCCTCCAAGCCCTGAAGGAAGCACCAAGTAGAGACGTGATGTCACCTTAATTATTCCTAAACCTTCACATAGCAAATTGGTCAGCTTTATTC
ACCCAGCATAGGGGAAGACAT1TCCATTGGGCACAGTGAGTTATAGTGAGTCACAGTGTATGGCTTATTCACTTGAGGTTGAGGCAAAGGCAGC
ATCGAGAAAGGGCTGGCTCTGAAGAAACTTCTTCAGAAGCAGGCCATGCTGTGGTAAGTTTCTCCGTTCACTTTATCCTTGCAGAGGAAGAATG
GAAGGATGGTGTGAATTGTCTGTCATCTTCCACCCAAGTGATCTCAGTTCTTAAACACTGAGTGACAAGGACTGATGTTAGTGCCCATGAGAAG
AAGGTATATGTTTCCTTTGCCAGTTTGTCTCCTGCTGAGACAAGCAGGAAAGATCAACCCCCCCCCCATAGGGCCCATGAAGGAAAATTCTTGC
CAAGTAGGCTCTAACCTCAGAAGGAAGCAATCACCAGCTCAATGCTGAAGTTAGCCTTTGTAGCCTTTGTGGCACTGTGGCTAGACTCAGGATA
ACTAAGACAAGAAGAGCAGGTGGCAGATGTTCAACATCATTTCCAACAAACTCCAGCCTTTATCAGCACAAAGAGGTAGCCCAATGCTCTACAC
ATTAGCACCTCAAGTTGTCACAGGTCTCTGTCTCAGGAAGAAAGCATCCATGGTTGTTGCTCGTTTTGCCAGAAAAAAAATGTCACAAGTTTAG
CAGCCACCAGGGTGATGGGACTTTTTTTTGGCAAGTTGGCAGCTCTGAACTGCCTGTGTTCCTTTCCAGCTTGTCCTTCATAGCTCGGCCATCC
CATGGACTCTCTGGCAGGACAGCCAGAGCGGTCTCTGAACTCAGAAGTAAAGTAACCTTTCCATGTCAGAAATTCTAGTGTCAGGAAGGAACTG
GGGACTGGGAGGAGACACAATGGGGAAACTGTTTGCTGCTTAAGACTGAAGACCTGAGTTTGAATCCCATAGAATCCATGGAAAGTCAGATGCA
AGAGTACACATCTGTAATGCCTTCTCTTCCGTAACAAGACAGGAGGCACAGCACCCCTAGACACTCACAGGACAGCTAGCTTGGCAATCTACAG
CAGCACAGGAAATCCCATCTCAAACGAGGTAGTAGGCAAGGCCCCACATTGACCTCCACGTGTGTGTCACGGCATGCCTATACCTGTGAAAACA
GAGGACCTAGTGTTCTTGACTTCTGGCCATGACTCTTTCCCGTGTTTACCATGCGTGCCCCACTGATTAGTTCCTAACACTGGGTACAAAGATG
CCCTGCGTTCTTTATTCATAAAATCTGAGCCAAGCCTTGTGTCCTGAGCAAGAAATGCCCGTCACCAGGGCGATAAAGTTCCTAGTTACTTGAG
GCTGTTATAAAGAAGAGTCCCACCTGCTTAATTTCAGGTGGAGGAAGTTGGGAATTTGTTTTCCATTTAGCAAGTGGCATGGGATAGGCTTGTT
CTAAGAGATTACACCCACAGTGCTTACATTCCTTCTGGATTATTCAGGCTTACCTCCTCTTTTAGAACAAAAACAAAACTAATGACAAGCAACA
TTTATTTAGCCTGCAGAACAACTCAGGATCACGTGGGTGGGCATGGTTTTGTTCTCTGATTGGATAAACTGAAATTTTATAGTGTGGCTTTCAG
GGGGCACACAACAAGGTTCACAGCATAATAACATTCACCTGAGTTTAATATAAACTAAGCAATGTTCAGGTTTAGAGTTTAACAATGCCAGTTT
AACCCCAGAATCAAATCACTTCATCTCCAAAGATCCAGAGGCTGGCTAGCTAGTGTCCTTTGCATTATCCGAGCAGGAAGACTGAAGTTCTGGG
GGGGTCAAAGGACTCACAAGGTCACTCAGCTAGTGTGTTAGTGGCACAGATGGTCACACTCAGGGTGCACCTCACTCATTCTCACCCACCATGT
CCTACGGCCTTGGTGTGGGAGGTCATTTAGTGTTCACAAAGGGAGACTTTCCTGAAGCATTACTCTTGGGTCTCTCAAAGCAAGCTTGTCTACA
GACTGGTGAGAGAGATAGTTACATCCTGTTTGNNNNNNNNNNNNNNNNNNNNCAGCCAGTGTGGGTGATTAAAGCTAAGGAACAAGTCTCTTTA
CTCCCTTCTCCTCTACCTTATCATTGACCTGCATTCCTCTGGGGACACTTCCCTGGGGGATGTGTTTTAATCTAGAAATGTCACAAAAACTGTT
GTCCAGTGCTCACAGGCATCCAAAGCTTTTAATATGAAACTCAACACTTTCTGGTGCTCTCTATTCTGAGCTACTTGGGAACCGTGCCTTCCAG
ATGATGGCCATGGAGGGTCAGGGTATGTGGAAGTGAGGAGTCAAACACTGCTGCCGAGGGCCATGAGGTAGACCAGTACTCTACAGGCCTCACT
GATGACAGTTGGCATCTCTGATGAGAAGCTGAGTATAGCAACTTTCCAGCTGACCTTTAACCCTTCCCTTCTATGAACTTTTAGTAAAACGCCA
TGCATTAAATGCTAACTCTTATAGAGAACAATCTGTAGGATAAAGGTGTGTGTGTGTGTCTGTGTGTGTGTCTGTGTGTGTCTACCATGTGGAA
GATATTGCTAATAGCACTGCATGTATCATGTAGTCATATAGCCTGGAAAGTGGTATGAACATTGTTAATCAGAAGACCTTTTTCACATGCCCAT
TTCTGTGCATGGCATAATCTCCATTCCTTCTCTCCTCCTTTCCCTACTTTTTCTTCCCTTTATGCCAGTGTTAAGAAAATAGAAAAGGAACAAG
GTTGTCCAGAAAGAGGTCCGATAACTATGGGGTGCACAGTAGCTTGCTTCTCCTGAGTGTGTCCTGTTGTAGGTATAGCTTCTGCCTTCCCCAT
CTCCTGTGACATGTGCTTTTCCCACCCATCACTTCCTTGCTCTATCTTTCTAAATGTAGAGCTGTAGGGGACTGGGCAGATGGTTCAACAGTGA
AGCACTCATGACACAAGCGTGAGAATCGGGGCTAGCATCCCCACAACTCACGTAAAAGCCACATAGCTATAGCAGCTTGCCTGGAATCCCATGG
CACAAGAGACTGAGATGGGGTGCCCCAGAGCAAGCTGGCTATCCTGACCAGCAGAAATAATGAGCTCCAGATCCAGCAAGAGATGCTCTGTCGT
CTAACATQGAGGGTGATCAAGTAGACACATGATACGGAACATGGTCTTCCACAGACACACGTGAACATGCATCAGCACTTATGCAAACATGCAT
CCATACCCACATCTACATTATACACACACATATACATACACACACATACCCTTCCCCCCTCAAAACAGTAAAGCCCCAATGAGATGGCTCAGTG
AATAAAATAACGTACTTGCTGCCAAGACTGACACCCTGCATCCAGTTTCTACTAATTCATATTGTGGAAAAACAGAACTCACCCTTACCAGTTG
TCTGCTGACCTCTCTATGCACACAGTGGCATGTGGACCAACCCTGATTTTTCTTTTTTTAATAATTTAAGAAAATGTAGACCAACAGCTCCAAT
TTCTGATTGTGGCCCAACTCTATAGCTCGAGATTTGAACATGTAGAATCCATCATGCTTTGGAACCATTGAAATCCATTCTCTCTATGCAAAAG
GAACCACTGTTGACTCAGGCAGAAGTTAAGCTTCCTCTGTCCCCCTTTCTTACCACCTGTCTCTCTGTCCCACAGAGGAAGCTCACCAGTGTGG
GAAGGGCCTTCTCATCCACTGCCAGGCCGGCGTGTCCCGATCCGCCACCATCGTCATCGCCTACTTGATGAAGCACACACGGATGACCATGACT
GACGCTTACAAATTCCTCAAAGCCAAACGACCAATTATTTCCCCGAACCTCAACTTCATGGGGCAGTTGCTGGAATTTGAGGAAGACCTAAACA
ACGGCGTGACGCCAAGAATCCTTACACCAAAGCTCATGGGCATGGACACACTTCTGTGACAACCGCCAGCACGGAAAGGCCTGTGCTCTCTCAG
GAGACGAAAAGGAGGGAAGGTGGATTCTAGCTTGTCGCTCTTCTTTCCTTTCCTTTTCTTTTCTCTTCTCTTCTTTTCTTTTCTTTTCTTTTTC
TTCTTTTCTTTCTCTTTCTTTCTCTCTTTTTTTTTAAGGTTCGGAGTGTTTGTGAAAGTAAACAAACTTGTCTAAAGACTCTATTTTTAATAAG
TGTAAGAAGACTGTAACTTTTGATGCGAATGAGACCCACTTCTCTTAAACTGGTATTGCAGTTAGGTTAAAGAGGTCTTTTTTGTTTTGTTTTG
TTTTTTTAAGCCAACAAATAAAAATATTATAAAACTTGTTTCTTCCCAGTTTCCCTTGTAAGCTTATTGTAGAGTTTTGAGTAAATAGTCTGTG
CGGGCTCATAGACCGAAGATACCACCCTTCAATTACAAAGAACCAAAAGGAAATGGAAAAGACTCCAGACCTGGAACTGACTGCACAACCATGT
CAAAAGCAAACCCTGCCAAACCGTGCCCAGTTGTGCTCACTGGTGTGAAGAAAGGGCCGGCAAGCTCAGTGGACTCGGCTCGCCTCACACCTTC
TCTGGGGAGACAGGCTAGTGTCGGTTTTGTGTGTTCTATGCTCCGGATATCTTTTCCCCTTCTGTGGGTCTGAGAGAACTTTTTGTTTGTTTGT
TTGTTTGTTTGTTTGGCACAAGGAAACTGACCATTCTACCATGTGCCTTTGCTGGCACTCGTGCAATAAAAATGGTGTTTGGAGTGTGCAGCTG
AGGGAGAGCTGGCAGCTGAGTGAGGAGTCGCCTTCCAGACAGGCATTCAACAGTACCAGTGGCTTCCCCTCCCTCCTTTCTCCTTCTCCCCCCC
CCCTTTCTTCCTCATCCCCTTTTATTTGTCTTTCTTTTGATTTGATTCTGGTTACAGTGCCATACACCTTGTTACATATGTATATCAGAATGTA
AAAAAACAAAACAAAAAAAAAACAAAAATTTATTTAAAAAATATTTTTCGCAAAAAAAAAATATCCTTGGTGTGTTTTTGTTGATGGTGTAAGA
ATTTATTTTCATTATATAAATGAACCTCGTTTGGGGATGTGTCATTCTTTGTACGTGCCTGTCTTCTCTGGTTAGGCTCATACCTCTCAGCACG
GGTTTGTATAAAGAGATGGGGATGAGAGTGTAAAGCGGGAGGGACCGCTCACGGAGGGAGGCTAAGGATCCCACCACTGATGCGGTCCTCTGCT
TCCTCCCCATCCTCTTTCTATGAGACAGCAGACTCATCATTGACCAGCTTCCGCCAGACCTCATAACCAAATCACAGCCAAGCCGAGGATTTAG
AATATACAGTTGTCTCCCTGGTAACTACATGTAAATATCAGTGACAATCTGCCTTGTATGGTTTTCACAGTCCCGTCTCAATTGACACTAGCTC
ACATGACACTTTTTAGGGACATTCACCCTATAGTAACAATAGTACAAGCTGCAAAAGATGTACTCTTCCAGTCTTTTTCCCAGTCAGTAGAAAT
CAGCTCTGTGTGCACACGGACTTTGACAGTGCTGGCTATTCAGCCCACAGACAGTTGGAAGTGGTGTGGGAAGGATATATAATCCTACGGTTTT
GTACGTTTTATTAGACAATATGTATCTAGAGCGACTTAGCCACTGGGAAGATTTGTAGTGCCCCAAAGTTTGCCTGGTGCGAGTAAGCGCTGAA
GAATTCTGGCATGTCTGGCTCCCAGTGACTACATTTGACATAAATATTTTTGTGAACATTTGCTTACAAATGTTACACCAGAAATATTTTGACA
TTGATTTCTTTATTCAAATAGCTTGCCTGCCAAGAGTCATTTAAATTTTTCTTTCATTAGAAAAGTGGAAATTGAACATAATAGGAACTGAGAG
CTAGCTGGAATTACGTTAGTCTGATATAGAATATCAACTAGTCAAAAAGTAAATCTCTTTTTTTGGCATTTCCGTACTGTCAACAGTCTTTTTG
TTTTGTTTTGTTTCTGTCAACTAGAGCAAACTGAAAGAGTCTGATACACAGTGACTTGGGATGCCTGGCAGGCTTGTCATTTTCTAAGTCTAAA
AGTTGGAGTCCTGGTGGCCTATAAAATTCTCTCTCTCTCTCTCCCCTCCCTCCTCTTCTTTCTCTTTGCTTTTPTATCATGTCTTCTGCAAACG
AAGGGAAGCACTCAATAGCTACATGGACATGATTTGTGTGTTTCGAAGTTTATGAGCTTAGAGGAGAAGGTCTGCCTTGGGGCAACAGGAATCC
TGCCAACCAGCATACCAAGAATTCTGAGAGGGACTCCAGTCTTTTCTCCTAATCTGCTCTGAGTCACTTCACCTTCGTTTCTTCTTGAGGAATG
TTAATCACTTCATGAACCCACTGGATCAGCAGAGCCAAGGCTCTGGAAAGCAGAGTCCCCACCATTCTGAGCCTCTCACTAGCTGCCTGCAACC
TTCAGGATGGGGTTGGGAAGTGACTGTGTCTTCTGAGAATGCACAGTCTATCCACCTCAAGAGTCATCCCCACGGCCTGTCCTGAAGGAATGCA
GTGAGCTGGTGGAATGAATCCAGAGTCTATCTAATGCCCAGTAGAGTAGGCGTCACTGCCTTGGTCTGAACAGAACAACAACTCCAGATTGTCC
ACATTTATTTCAATAGCTGATAGTTGCTTTTCCAGTGTCAATGAGTCTAGACAGCTTAGTTCACAGATGAGCTCCTGCTGGTGACATTTGGACC
AAGGTAGATGGAGTGGAGAGCGAGGACGGAAGCTGACAAGAACGTGTATGCAAAATAATAGAGGAAACATTCTTAGGCTGCAGCCATCACTGAT
TTCTACTCACTGGGAAAAACACTGGAAGAGTACATCTTTTGCAGCTTGGACTATTGTTACTATAGTGTGAATGTCCCTAAAAAGTGTCATGTGA
GCTAGTGTCAATTGAGACGGGACTGTGTGGGCAACAAATGACAGATGACCCAGAAAAGCTAAAGAGAATAAACAGGGGGAAATGGGAAAGTAGA
GTAACGAGAGAATGAGTACTCGTGTTGAATTGGGATCCAAATCATCTGAAAACTGAGACATAGAGAAGCAGTGTTGATCTCTTTGGGTCTTCTG
AGGGGTAAGCATGGTGAGCCAAACAAAGATCAGACTGTCTTAACTTTCACCTTATTTGAAACCTGTCTCTTGGACATGAACTATAGAGATACAT
GTGGAATTATTTCTAACCTCAAATCTCAGCTGTTCTGGGGCAGGGCACAGTAGAAGATATCGAAGCTCTGGTACTCCAAAGTATGAGGAATAGC
ATATTCTAGAACTATACTCGTGCCAGTTTTACCCACCTGCCCTTTGGAGGCAGCAGGGACTGACAGCCATATTGTCACTTGTTCCTGATTTCTG
TCTCTGGTTACCTTCATCAATGGTGTGAAATAACCAGGCTTTGGAAACATTCGAAGACTCACTGGCAGAGTATTGCTTCTGTGTGACATCGCCT
GAAAACTCTGTGTCAATAACCCCATGGGAGAGAAAAGCTGCACATGAAATAGAGGGGTGTAAAGATGAAATTACAAACGTGCTGATCATGTCAC
TGGACGGTTGAGCAGCAGAACAGGGGAGAGCGTAAGAGCTGAGCTCATGGCTCCTGCTAAGTCAACGTAACTCACAGCGTCAAGGCAAAAGCAT
GCTCACTATAATTCTCCCCATTGCCCTTCTAAAATGGGATGGCTGCTATTTGTCTTTGTTTGTTTGTTTCCCCCACAACTTGATGCAAATTTCC
TCTCAGACTTAGTAACCCAATGTGAACAGGCAAGGGAAATGTGGGAAAAGCCAGTTCATGTCTAGGATGATTTTGTCATCGACCTCATCGCGTT
TGGAACCTCCCTTTCTCACTGTTCATGCTGTTCCTTTGTCCTCAGTAAGCACATCCTTCATCATGGTTCTTTGCCTTCTGAATGGCCCAGACCT
TGACAACTGGGCTTGTATTAGATTGTTTTCCACTCATTTTATTATAGTAACAAGGCAACTACAAGCACAGCATCCTCTAAGCGACACTAGCATT
CCAAATGGACTTTTCCTTAACCCAGTACCAGCCCACCTTACTCGTGACAATCACAACCATGTCAGTCAGTATAGTAAAATATATTTTTGCTTGT
TTTTGAAAAGGTTTATTTCCGTTTWAATGTGTGTGTGTGTGTGTGTGTGTGTGTGTGTGTGTAGTATGTGTATCTGAGTTCAGGTACCTTGAGA
AGCTAGAAGAATGTGTTCCATGCCATGAAGCTGGAGTAACAGGCAGTTATGAGCTGGCTAATATGGGTGCTGGGAACCAAACTCCAAGAGCGGG
ATATGCTTGAACAGAGGAGCAATGTCACTAGCCTATGTCTGATTCTTAGTGTAAGGCACCCAAGGAAGTTTTAAGTGTCAACTTCTAGCACAAT
GGATCCATATAGCATCCCCCAGTAGAGGTGTCTTTACCTTAGGAACTAAAACTTCTACATTCCAGGGTAGAAAGAACAAGCTGTGTAAGTGGAT
CATTAATGACATTTGTAAGAGACGTTTGTCCTGTTTCTGTCCCTTGATTCCTGCATTTAAGTATTCTTAGATAAGTGTTGGGGGTTGGTCTGGT
GCTGCTATGTATTCAAATGATAAATACTGGCCCCAAGATCTGGTTTATACCCCCCAGCTGATCCTAACATCAAATGATGCTATGAAAACCTTGC
TCCCCAATTTAAAATATTGGTTCAGTAAAAATGGCTACAGTCAGTTACTGAGGAGAATAGAAAGAGGCAGCCTTTCCATTTACTGGGGCTGCGG
GGTAGCAGGTAGGAGGGAAAATGAGTAAGAAAATGAACATGGAGAAGACAGAAGCTCTCCATTAGATGTGATCGACCAGGAGCAGGTGCCTGAG
TGAATAGTCCCCCTCCCCCGCAAAGGACAGATTGCTGGAGCACAAGTAACTCAGGAGAGACTCAACAGGATAGTATTTGGGGAGAGGTGATGGG
GAAGTAGCCAGTCTAGCATTTCAAAAATAGATTAGGCCTAATCCCCCACTAATTCCGCAAGAGCTGATTAAATAAATCCATAGGACTCTGCCTC
AGTTACTGGCAGGCTGAGTGGGTCATATTAATAACTAATAGAGTTTATTAAACTCCATTGACAAGGAGAAACAACAACATATACTGACTGATGA
CTCTGACTTTATTGCTTCTTAGGGGACATTACTCTAGCCCTGTAACATATTTCCATTCGTCCAGCATAGCAGTAGACTTGTTAGPACTCTCTGC
CTTTCTACTGCTTGAAGTGATAGATAACATGGTAGGACTAATGAACATGAATCTGTTTCCATACACAATTCACCACAGAAGGAGGTTCCTTAAA
GGCATAGTGTGGTATGTCGTGGTGGTGTATAAGAAGTACTCTCTGAGTGGCCAATTGCTGGGTTTGGCAAGGTGTTTAGTAGGAAAGACAAACC
CATATGAAAAGTCAGTTTCGTATGAAATGAGAAGAACTTGCCCTCCAAGATAGAAGTGGTTTTGTCATCAGATACCTGAGTAATTCCCCCAGAC
GGTGGTTTGCTAGTCCAGGAAATGAGTGCTGCCTCATTTGGAGTTGGTTGCACATGTATTAGTTGCTATAACCCAACTAGACTTTGATGAGTAC
ACAATCATGTGCCTAATTGCAACCCCATGTTGACCAGCATGGGGACTTTGATCAGAACATCATTGATCAATAGAGAGGGGAGGCTGACAGAATG
GCTAAGCTTATCTAGTCTTAAGATACTTCTATCCTAAATCCGTGTCTTGGTGGGCAGGCAACAATCCATCCCACTAGGAGAAACTGATTCCTTG
CATTCGTTTCAAAACTTTCCAATCTGATTTACTCTGCCATCCTTCTTTAAAGTCTTTAATAAGCAATGAAAACTGTCACTGCTGCCCATAAAGC
AATGTAGGTTCATATTGTTGCTCCTCTCTGTGTTTAGAAAAAAAAAATCAACAATGAGATGCAAAGTTCTGCCTACTGGAATGCTTTCCTTTTA
CCTCTTTTTCTGGATCATCACAGAGTAGAGATACAATGATGTAGCCTTWCACTCTGGATGACAAGCTAGTATGCAGAGCTTTCTTTAGACAGGG
TTAATTTGTTTTTCaTGACTAGTCCCCAAGAAGTTCCCATGACTCTAGAGGTATAGGTTCTAAGACATATTGGAGCAGAGATATGGGTCTGTGG
GTCAGTGCTTATTGATTGGCTTGGCCTTGAGAGAGTGTATTCCATGCCCATTGATAATGAAATGATACTGTTCATGCTCTATGTACATACATGC
TGGGCTCAATAATGCTTAGCTCATAATGGGAAATTTGGGTTATATTGTAACTTTATGGTTCCAGATCAAGCTTCCAGPATCAAATCATATCTAA
AGAAAGGATAGATTTTCTGAAAAAACAAAACAAACCAACCAAACAAAACTACTGCCATAGTATTGCTCCTGAATTCTAAATATCTATTCTGGCA
TCTATTTTGTGATTTGCTTACAGGGAAATGGCTCCATGCAGTATTGCTGTCACACAGAGAGACCTTTAGTGCCACCAGATTTACTGGATCCGGG
TGTTAAAACATCTAACATGGTAACCTTGAGCCGCAATAGAGACTCCATAGTTCTGGATTTTACTCAAAAAAATGATAACCTTTTATCACAGTAA
ATGAATTAATTTTCCTAAATCTAAAAGGGCCCTTATGTTTTCTTCTCTGTGGTAGCAAGTACCACAGATGGTACTAATTTGTTACTTTGTCCTT
AAAGAAGATGTATTAGCATACTTATTTTAAAAAGATGTATTTATTATATTTATTTATTTTTTTAAAATTTATTTATTTATTATATGTAAGTACA
CTGTAGCTGTCTTCAGACACTCCAGAAGAGGGCGTCAGATCTTGTTACAGATGGTTGTGAGCCACCATGTGGTTGCTGGGATTTGAACTCCGGA
CCTTCGGAAGAGCAGTCGGGTGCTCTTACCCACTGAGCCATCTCACCAGCCCCTTGTTATATTTATATCAGTACACTGTAACTGTTTTCAGACA
CACCAGAAGAGGGCATCAGATCCTATTATAGATGGTTGTGAGCCACCATGTGATTGCTGGGAATTGAACTCAGGACCCCTGGGACAGCACTCAG
TGCTTTTAGCCATCTCTCTAGCTCCCTACTAGCATTCTTAAAACCACTAGACGTCTATACATTCACTGGTGATAAGCCCCTGAGTGTTTCAGAA
TGTGTTTCTCAACCTTTTCATTACTAAGTAGAAGATAGATAGGTTAAGAGCCAAGCTAAGCTCATTGAGAAAGAGATGACTCAGGTACCCATGG
TCTCTGACAAATCCCTGTCTTATCTCAGCAGCATATCTACATCAGTTATTTCATTGTGCTCATCAGGCACAGTGAGTGCTATCATCCATCCATC
GTGTCTGAGGGGATGGATACACACCTACGATCCCTGTGTACTCAATTATGATACAGGGATATCAAGTTGAAATGGACCTAAGGTAGGATAGTGA
TGGTTACAATATTATWCATTCTGAAGGCAAACTCCTTCTAATGGCCTTAACTTGTGCTTCAGAAACATTTTATGTAACGGAATAATAAGTAATG
TAGGATTAGAAGGTGATATAGGCTGGCTAGCGAGGCTACTCTAGAGTTGGAGTACAATTACAGTCATAGACAATATGTAGAGGAATGGGAGTAA
CTATATACTGTAAATACAGCCATCTGGATTTCGTCCATGCTCTATAAATAGCTAGTTCCTGGTCTTTTTAAACAGGTATACAACAGCCATAGAG
AGCTAAACATTTGCTTTACCAATACGTACTTATGGGTTACCAGAGCCATGTTGAGTGGCTCCATTAATCAAACCATGTCTGGTATAACATCTCT
AACAGAAACCAACAACTGAAAGACATCTTTAAAACCTACTGTTCCTTAAATTCATCTCTTTTTTGTGTAGGACAAATTATTGACAATAAAGGAG
GAACATGTAACGAATGCCATCTTTCCCTTTTGTCTCAGTTCCTGTCGCTGTCACTAATCTGTTCCATTCTTTAAGAGATCAGCATTGCTCTTGT
GTTTGCTACCTTAGTAACTTGTAGCTTCATAACAAACTATATCAAAACCTAGTGGCATAAAAGAATTCAAATTGTTGGGTTTGTTAATGAATTT
GTTTCAAGTGTGTTCAACAGACAGGTCACTTGAACTATAAACTCAGAACCAGAAGTGAGGCAGCTTGTTGGGGCTGGAGAATGTGTTTCCAAGG
CCTTGCAAGTGCTTTTCTAACTGACTGGGAATTCAGCCAGGGTTGTCCACTAAATACCTTGGATCCTCTCCTCGAGGATGCTTCGQCTTTCTCA
TGGTAGCTGCTTTCCAAGTGACTGAGATGAAAGTTGCAAAGCTTCTAAAATCGTATAAACACGGATGTCTCAGATCACTTCTGCAAAATCCTGT
TACTAAATAATTTACAAGCTCAAGTTCAAATTCAAGGAGAAAGCAATTGGATTCTACCTCTTAATAGCAAGAATAGCCAAGGATCTGCAACATT
TCTTATCACACTGTAGCCCCCTTTGCACAACCA2TCAGTATGTGAAAATAGTTTACACGAGGCAGGGCTGTTGGATAATGACAGTAAGTCAGGC
AATGACATCAGCTTCCTCCAAATATTGCTTCAAATAGTGTTGTAATTCAAAGCTAAAGTTGAACTGTTTTTTTTTTTTTCTCCTCCCAAACCAA
GCCCTGASGATAAGGATAAAGATCACAAGGGGAAGGTCGCATCAGCTGATGACTGCCGCCCTCCTGCTTCTGATGGTATGCCAGCTCTCAAGGC
CACACGACTCACAQTCTTGTCTCATCCAAATTGTATTTGGACAGTATACTACATTTTTAAAGTTACTQATCTAAGAGGCATQCGATACTCCATA
ATTCCTCCCCAAATCCCTGSGCATCGTTGTGACCTTTAGCTTGCTATATTTCAACGACAAACATGAATGCACCCIAAAGACAGAAGACAGACAG
ACAAACAAATAAGGAACATTATGTGGATCTGTCCTGTCTCTACATATCATGTGATGCCTAAGGATTTGGGAACCAAAATATAGGTCTGCCATTG
GCTTTTGGATTGATCCCTTGCCGTCACTGGGAGATAACTGTCAGATGTGTCTTCTGGCCGGGGGGAGGGTGTCTCAATTTTTTTTTATTAACCC
ATTGTGTGACACTTGAATTAAGCTTTAATTTCTCTTTGTCTPCTTTTGTTTTTTGTTTTTGTTTTTCTGAAGCAGATCTCAAGGTAGGGGTTTT
AAAAGAAGCAGCTGATGAAATGATCCAATAAAACTGGCAAGGGAGAGTAGAACTGAGGCCAAGATGGAGGGAAAGGGATGGATAAACAAATTGG
AAAGTAAGATCTTGTTTCCAATGTAGAAAGTGAAAGAATTATCCTAGTGAGGGTGGGAGCTGTTGCATTTGTTCATCAAGGTTCCTTAAGTAGA
CAGATGATGGATGCCAGTCCCAGTACTTCCAGTCTTCTTAGAGGGAAGCCACAGAGGCCAGAGGAAGTCCTCAGGGAATCAGAACCACCCACAC
TTCGTCCTAGGAATCATAAGTGATACAAAAATGAACTGAACATGAGTGTCTGGGCTATAGGCCTTACAGCAGGTAGAGTTCCTCTGAGCCCTTC
ACTTCTGACCACTTTCCCAGTCCCATTGAAAAGGATAAGGAGAGGACTTGTGGGAGGATAAGGATGGGTTGTGTAGGAAACAGATCTTGTCTGC
TTCTTTCCTTTCTTCTTGAACTCAAAGACATGATTTCTTACCAGCTGGACTCTGAACCCTTTATTCTTTCATGTCCTGTCTTTTTCACCCTCTC
ACTTTTCTTTTTTATTGAAAGAGTTAAACTTGCTGAAAACCCAGTGGGCTTCTGAAGAGTCTCTAGTCTCTAGTGTTGCCTTCCTACGTTCCGC
CCCTAGGCCTCAGATTCTTCAGTTTCTCACTCATCCCACACCCATTCCCTTCGTCTGGCCGGTGTGCTTTCGGTAACTTAGATGTCTGTTTGGC
AATATTGAAAAGAGGGTAAATGGGGAAATGGGCTGAAAAAGAATTCATCAGAAAAAAACACTCTAGGTTGGTACTAAACTCAGACCGAAGTCTT
GCTCATCAGGCTGCTGAGCACTCCACAGGCATGCATATCAGATTGATGATTACTTTAAGAAATGATTAAATTATAATTATTTCCTCATGACTCA
AGTGTGGATGAATGTGAAGTTTAAAGAGAAGCATGAAAACCCCAGTCAATGATCTTTTCTTCATTATCACGTT
MOUSE SEQUENCE-mRNA
AAAGTAACTCTGTGTTGCTGCTGSQGATCAATGAAGCTGAGTGAATSGGGGCGCACTGCGCQAGCGCATAGCTGGAGCGGGCGCCAGATGGTAG
AGGGCTACGCTTATTGATGAAACTCTTGACTTCTTGAATTGCCAGTTTTCAGCCTCCTCATGCCTCCATCTCCTTTAGACGACAGGGTAGTAGT
GGCACTATCTAGGCCTGTCCGACCTCAGGATCTCAACCTTTGTTTAGACTCAAGCTACCTTGGCTCTGCCAGCCCAGGTAGTGGTAGCCACGCT
CCTGTCCTTGCCGCCGCCGTTGTGACCCTCAAGGCTGCGAATCTGACGTATATGCCCTCATCCAGCGGCTCTGCCCGCTCCCTGAATTGCGGAT
GCAGCAGCACTAGCTGCTGCACTGTGGCAACCTACGACAAQGATCATCAGGCCCAGACTCAAGCCATTGCTGCAGGCACTGCCACCACCGCCAT
AGGAACCTCGACCACCTGCCCTGCTAACCAGATGGTGAACAACAATGAAAACACAGQCTCTGTCTTAAGTCCATCCGGTGGCCTCCGCAGCCCT
GTGTCAGGGACTCCCAAGCAGCTAGCCAGCATCAAGATCATCTACCCCAATGACCTGGCAAAGAAGATGACCAAATGCAGCAAGAGTCACCTGC
CCAGCCAGGGTCCCGTCATCATCGATTGCAGGCCTTTCATGGAGTACAACAAGAGTCACATCCAAGGAGCTGTCCACATTAACTGTGCCGATAA
GATCAGCCGGCGAAGGCTGCAGCAGGGCAAGATCACTGTCTTAGACTTGATTTCCTGTAGGGAAGGCAAAGACTCTTTCAAGAGGATCTTTTCC
AAAGAAATTATAGTTTATGATGAGAATACCAATGAGCCGAGCCGTGTGACACCATCCCAGCCACTTCACATAGTCCTCGAGTCCCTGAAGAGAG
AAGGCAAAGAACCCCTGGTATTGAAAGGGGGACTCAGTAGTTTTAAACAGAACCATGGAAACCTCTGTGACAACTCCCTCCAGCTCCAAGAGTG
CCGGGAGGTGGGGGGTGGTGCATCTGCGGCCTCGAGCATGCTACCTCAGTCTGTCCCCACCACCCCTGACATCGAGAACGCAGAGCTAACGCCC
ATCCTGCCCTTCCTGTTCCTCGGCAATGAGCACGATGCTCACGACCTAGACACCATGCAGAGGCTCAACATCGGCTATGTCATCAACGTCACCA
CGCACCTTCCTCTGTACCATTATGAGAAAGGCCTCTTCAACTACAAGAGGCTGCCAGCCACAGACAGCAACAAACAGAACCTGCGGCGGTACTT
TGAAGAGGCCTTCGAGTTCATCGAGGAAGCTCACCAGTGTGGGAAGGGCCTTCTCATCCACTGCCAGGCCGGCGTGTCCCGATCCGCCACCATC
GTCATCGCCTACTTGATGAAGCACACACGGATGACCATGACTGACGCTTACAAATTCGTCAAAGGCAAACGACCAATTATTTCCCCGAACCTCA
ACTTCATGGGGCAGTTGCTGGAATTTGAGGAAGACCTAAACAACGGCGTGACGCCAAGAATCCTTACACCAAAGCTCATGGGCATGGAGACAGT
TGTGTGACAACGGCCAGGACGGAAAGGGCTGTGCTCTCTCAGGAGACGAAAAGGAGGGAAGGTGGATTCTAGCTTGTCGCTCTTCTTTCCTTTC
CTTTTCTTTTCTCTTCTCTTCTTTTCTTTTCTTTTCTTTTTCTTCTTTTCTTTCTCTTTCTTTCTCTCTTTTTTTTTAAGGTTGGGAGTGTTTG
TGAAAGTAAACAAACTTGTCTAAAGACTCTATTTTTAATAAGTGTAAGAAGACTGTAACTTTTGATGCGAATGAGACCCACTTCTCTTAAACTG
GTATTGCAGTTAGGTTAAAGAGGTCTTTTTTGTTTTGTTTTGTTTTTTTAAGCCAACAAATAAAAATATTATAAAACTTAAAAAAAAAAAAAAA
AAA
MOUSE SEQUENCE-CODING
ATGCCTCCATCTCCTTTAGACGACAGGGTAGTAGTGGCACTATCTAGGCCTGTCCGACCTCAGGATCTCAACCTTTGTTTAGACTCAAGCTACC
TTGGCTCTGCCAGCCCAGGTAGTGGTAGCCACGCTCCTGTCCTTGCCGCCGCCGTTGTGACCCTCAAGGCTGCGAATCTGACGTATATGCCCTC
ATCCAGCGGCTCTGCCCGCTCCCTGAATTGCGGATGCAGCAGCACTAGCTGCTGCACTCTGGCAACCTACGACAAGCATCATCAGGCCCAGACT
CAAGCCATTGCTGCAGGCACTGCCACCACCGCCATAGGAACCTCGACCACCTGCCCTGCTAACCAGATGGTGAACAACAATGAAAACACAGGCT
CTGTGTTAAGTCCATCGGGTGGGGTGGGCAGCCCTGTGTCAGGGACTCCCAAGCAGCTAGCCAGCATCAAGATCATCTACCCCAATGACCTGGC
AAAGAAGATGACCAAATGCAGCAAGAGTCACCTGCCCAGCCAGGGTCCCGTCATCATCGATTGCAGGCCTTTCATGGAGTACAACAAGAGTCAC
ATCCAAGGAGCTGTCCACATTAACTGTGCCGATAAGATCAGCCGGCGAAGGCTGCAGCAGGGCAAGATCACTGTCTTAGACTTGATTTCCTGTA
GGGAACGCAAACACTCTTTCAAGAGGATCTTTTCCAAAGAAATTATAGTTTATGATGAGAATACCAATCAGCCGAGCCGTGTGACACCATCCCA
GCCACTTCACATAGTCCTCGAGTCCCTGAAGAGAGAAGGCAAAGAACCCCTGGTATTGAAAGGGGGACTCAGTAGTTTTAAACAGAACCATGGA
AACCTCTGTGACAACTCCCTCCAGCTCCAAGAGTGCCGGGAGGTGGGGGGTGGTGCATCTGCGGCCTCGAGCATGCTACCTCAGTCTGTCCCCA
CCACCCCTGACATCCAGAACGCAGAGCTAACGCCCATCCTGCCCTTCCTGTTCCTCGGCAATGAGCAGGATGCTCAGGACCTAGACACCATGCA
GAGGCTCAACATCGGCTATGTCATCAACGTCACCACGCACCTTCCTCTGTACCATTATGAGAAAGGCCTCTTCAACTACAAGAGGCTGCCAGCC
ACAGACAGCAACAAACAGAACCTGCGGCGGTACTTTGAAGAGGCCTTCGAGTTCATCGAGGAAGCTCACCAGTGTGGGAAGGGCCTTCTCATCC
ACTGCCAGGCCGGCGTGTCCCGATCCGCCACCATCGTCATCGCCTACTTGATGAAGCACACACGGATGACCATGACTGACGCTTACAAATTCGT
CAAAGGCAAACGACCAATTATTTCCCCGAACCTCAACTTCATGGGGCAGTTGCTGGAATTTGAGGAAGACCTAAACAACGGCGTGACGCCAAGA
ATCCTTACACCAAAGCTCATGGGCATGGAGACAGTTGTGTGA
HUMAN SEQUENCE-GENOMIC
TACATTTTTAGTAATACAGGACAGGATTGCAGGACACTTGGTACAATTCTCATCTTTTAGGCTATGCTTTGCTCTGTCCACCAAAGGGGAAAAA
GGGTGTATTAGGATTCTCTAGAGAAACAGAACCAACAGGATGTGTATGTGTATAAATCGAGATTTATTCTAAGGAATTGGCTCATGCAATATGG
AGGCTGGCAAGTTCAAAATCTGCAGGGTGGGCAAATCGGCTGGAGAGCCAGCAAAGATCCAATGTTGCAGTTAAAGTCTGAAGGCTGACTGGCA
GAATTCCCATTGCTCAGGGGAAGTCAGTCTTTTGTTCCATTCAGGCCTTCCACTGACTGGATGAGGCCCACCCAAATTATGGGGGGTAATCTGC
TTTACTTAAAGTCCACCCCTTTACATGTTAATCTCATTCATAAACACCCTCGTACAAACAAACACCTTCACAGAAACAAGCAGAATAACACTTG
ACCACATATCCCATGATCCAGTCACGTTGGCCACAGAATTAACCATCAGAAAGGAAGATTGCACTAGCTTAAACATACAAAGGCGAAGTTCTGC
ATCCACACAGAGAGAGACACAAACTACTCCTTATACCATGGGACTAGCGGAACTTGTCTTTGAGGTTCTCCTGGGTTTAGAAAGAGGGGCCCCA
GGTGTGTGCTTGACATCCCTCAACTGTACAAAAGCAGTTGGCGCCTGTCTAAGCCAGTGCAGCCTTCCTTAACATGCACCAACACAGACAGCCC
TATTCTTTCTTTTCTATCCCAGGAGGTGGTGGGGCCCAGTGTCTGCTGAAGTCAATCAGAATGAGTTTTAGTAATATCACATTTTTTTTTTTTG
CCAGCAAACATTCAACAACCCCTCTGTAACCAGATCCTATTCTATTTAATAGCTTGTGAAATTGTAAATTTCTGGAAATAGCAAAATACCCTCT
ACTTGTCTGCTCTTTGCCTTCTTTCTGCCCAAAGTTACCCTTCCCAACTCTGGAGTCAAAATACAAACTAAATTTTAAAACAAAAGCAAACAAA
ACAAAACAAAAAACCCAGTCAGCCCACTGTTCTTTTTGCCWGCCTGCCTTACWTCTTTCAGTCCTTCTTTAACATGCTGGGCAAGTTTGATGAG
TTAAAATGTCTGCCTTTAACTCCTCTAATCCATATTCGGGGGGACTCATGTATTACATGAAGATTAGGACTGTGTTGAAAAGGGAGTCCAAATC
TATTGGCATTTCTATTCTGCAGAATATAGAAAAGACACTTCATTGGACTGTTTGCTATTATTAATCCAATTTAAGGTTTAGAATGGCGGTTCAT
AGAGTTGCTTCAGTTTTCCTAATTACTGGGAATAGGAATTGCAAACTGAAGCATGAAGGAGCTAAGAGTCCTTGTGAATTATACATCTTAGTTT
ACTACTGTAGAAAAATGATACAGTTTTTCTGTCACCATACCTGCGATGGTGAGCACTGGCAGATGAATGAAGCACAGCGCCAGCTCTCTGGCTA
CTTTCTGATCCAATCTATTTATCTTTCTTGCATTCTAAATTTTTGTGACCTGCAAGTTAATGGCAAAAATAACATPTGCTTCTTTTCTAACATT
TCTACTTGAACTAAAATTTTAGCCAGTTCCAATAAGGATTTTTCTCTTACCTGTTGAATAACTCGAATTGACTAGACCTGACCTGATTTGGATT
AATTTGAATACCCCTACTATATGCCTGAGTGGTACTGTGGGCTCAGTATTTAGCATAGCATAGACACTTAAATTCACCCAGAAAGGGTTTCATC
CACCATGTTTGTACATTCTTGGCCCTTGCTGGTCATGAGTATAATTACAAAGTGTTTGCTTTATTTACCAGCTAGACCAGGGAAAACACTGAAC
CCTCCCCCTGGGTGTGAAATCCAGGTGGACTTGGGCCCATTCACAGAGCATTAGAAACAGGACTTCTCGCGGTGCTGCTCCCCACCTAACCCAG
AATCAGTAGCGGCATTTCCTTTCAGAGCACAACCACCAGGAAGACGAAGAACTCACTGATGTCCACGCTCAGGTCCTTGATTAAATGCCTTTCT
GAAGAAAGCTATTTATTGGCATCAGTCACATGCTAGAGAAAATAAACACTGGACAATCCGTCTGTCAGGGAGATAAACACCAGGAAATGCATGC
CTTTGAGAATTACAAGAAGGAAGATGTTGAACGAGAAAAGGAAGATACCTTCATCCTCCTGAAACTCAGTGAACAAGAAGAGGAATTTTTTCTA
AACATTGAAGACAATCATAAWTGCAAAGAGCATPGACCAAAACAAAAATCAAGTACGGTATGGAGTATAATGAGATGGATTATAATGAGAAAGA
TGCTGATGTTTTCTAAATTCTATGGGAAAGAAATGGAGTTAAACTGTAAAGAGGGAAATATGATCACAACAAAACAAAACAAGTGGTGTGTAAA
CAACGTAGCTCCCCAGAGATAGACACTTGGTTAATATCTGCATAAATAATTCAAAAGAGTAGTCAATTTAGGGACTTTGGAGGTTTCTAAGTTT
CCAGCTATTACAGAAGGCCAAACCAGTAGCTAGAAAGCTATCATCAGATGGACCAGGGTATGCCTCTAGAAAAATGAATAATTCAAAGGTACGT
TCTTGCTACAACCTGAATATTTGTGTCCCCCCAAAATTCATATGTTGAAATCCTAAGGCCCAAGGTCATCATGCGGGCACAACCCTCATGAATG
CGATTAGTGCTCTTATAAAAGAGGCCCTAGAGAGAACCCTCACACTTTCCACCATGTGAGGACACAGCCAGAAGTTAGTAGTCTGTAACCCACA
AGAGAGTCCTTATCAGAACCTGATCATGCTGGCACCTTGATCTTGGACTTTCAGCCTCCAGAACTGTGAGAAATAAATTTCTGTTGTTCGTAAG
GCATTCAGTTTATGATATTTTGTTATAGCAGTCTGAATGGACAAAGACAGTATGTTTAAAAAACATTCTCAGTTCATTCATTCATTCAGTCACT
CACTCACTGAACCGAAGCTTAATGAGTTCAAACTACGTGCTAGCTCCCGGGCCACCGGCTAGGGATGGGACGGCAAACAAGACATGGTTTTTCT
AATAGAAGTGTCATAGGCTCTTCCTTGAATTTACCAGTCCAATCCGCAGTAGCAACCAAGCCATTCACACAATTTACGGCAATATTTGAGACTG
TTCTGGAAATAAAGTGGAGGTCTGGATCCATTCCCCAATGTGGAGGCTGCGGTTGTAAGAGTGAGACCACATGGAGATTCACGTCATGATGGAC
TTAGAAAAGTTCAGAAGAAAACAAAAGAAAAAAACAGCAAATTCTTATGAACAGGCAATTAAAAATATACTGCAGTCTGGAGACTGAGGGGGAC
ATGATAGAAGTTTATAAAATCACAACAAGAGTGAATGAAATAACACAAAATGGTTAAAAGCAGATGCTCTGGTGCCAGATTGTCTGGGTTTAAG
TGTTGACTCTGCTGCTTTCTAGTTTTGTTGACCTTCGGCAATTATTTACCCCATCTGTATTTCTCTTTTCTCATCTTTAAATTAAGCTAATAAA
AAAGTACTTACTTATAGATCGATCTGGCTGTGAGGATTTAAGTGAGTTAATGCATGTAAATCACTTACAGCACTGCTTAGCACAGAACAAAACA
CTTAGTAATATCTTACCTATGATTATTTTTTCCCAAATTCTAGATCTTTAAAATCAGTTCAGTTAATATTCAATTGTAAAGATAAAGTACGTTA
TGATGCACTTTGTTGTCTAAAGGAACTTCTTATATGTAGAAGTGGTACAGTCTGAACCTACTAGGAGATGCTGACAACGTTAGATTAATTCTTT
AGTGTTAGACCTGAAACAAGTCTTGAGGGGGTTTATGAATGCTTTGCGTGCATGTTTACCTTATAACTTTGCATCCTTCAGTAACACAGTTGGA
CCCAGCCTCCTGGGGTTGAGAAACTGGCGCTGACAGCATATCGTAATGATAAAACATAATTGCTCAGTTTAGTGAAGGGGAAAATGTTAACAGT
CTTTGATGTAGCAACACATAATAATAACAGGCCATCCGCCACAGAGGAGGGCATTGTTGGAGACCCAAGGCCAGTGAATGTTTTGTGTGTGTTA
TTTTCATAAAGAAAATAAGTTTACATCCATGGTCCACACACTTGGGCCTGAGGCAAAATCCTCTTCTGAGAAAGACCACCACATGTTTCTGGGG
TTACAAATAGGGGAGCAGTGAAGCGGAAGAAAAACTGAAAGGGATCTGCTTGCCTAAATGAATGACAGGGAGCATATTAATTCCCCTAACCAGC
CAGTCACCTCTGTGTGATCAGTAGAGGCCAGAGTGAACCTTGAAAAGAGGTCTCAGATCAATCATGCTTTGGCCATGCCATCCAGTGAAATTGC
TTTACAGGAAAAAAAAAAAAAAAAAGAGAGAGAAACAGGATTAAAAAACAAAACAGAGGATGGGCGTGGTGGCTCACGCCTGTAATCCCAGCAC
TTTGGGAGGCCGAGGTGGGTGGATCATGGGGTCAGGAGTTCAAGACTAGCCTGACCAACATGGTGAAACCCCGTCTCTACTAAAAATACAAAAG
TTAGCTAGGCGTGATGGTGCATGCCTTCTAGTCCCAGCTACTCAGGAGGCTAAGGCAGAATTGCTTGATCTCAGCACGCGTGGCTTCCAGTGAG
CCAAGATCATGCCACTGCACTCCAGCCTGGGCGGCAGAGTAAGACTCCATCTCAAAAACAAAAAAAACAAAAAACAAACAAACAAAAAAACCAG
AACCACCACAACAAAAAAATCCCAACCAGGATCCTTTCTTTAACTGAAATTTTACTTTGGAAGCCCATGAATAAGACATATATCTATCCATAGC
TATATCTGCATCTGTCAAGTGTGAAGGTGTGCAGGACAAGATTGTGGTCACAGAGGCTCCACCAGTTCACCCCTGCTCCCATTTCTATTGCCTC
CCACTTGCACTGATGTCTTTTACCCTTTGTATATAAACTTTTCAAGATCCAATGTGTATTTTAATCTTATGGCACATCTTAATTCAGACTAGCC
ACACTTCGAGTGGCCGACACATGTGTCAAATGACCACCACACTGGAAAATACAGATATAGAGCCTTAAATGGCTCAGAGCCTTTTCCTGAGTCA
CAAGCACACCTGCCCATGAATTTTGTAGGAAAGAACTCCAGGCCTCTTGTGTCCAAACTCATAGATAGTGGGACAAGCACATGGCCCTGCCTCT
GTTTCCATCATTACAGTAGCCACTGTCTTAGTCCAGTTGTGCCGCTATAACAAAATACCTGAGACTGGGGAAGTTGTAAGGAACAGAAATTTAT
TGCTCACATGAGTTCTGCAGGCTGGCAAGTCCAAGATCAAGGTGCCATCTGCAGGTTCACTGCCTGGTGAGGGTCCAATCTCTGATTTCAAGAT
AGCACCTTGTTGCTGTGTCCCCACAGGGGAATGGAAAAAAACTAACTCACTCCCTGAAGCCCTTTTGTAATGGCCCTAATCCCTTCCATGAGGC
CTCTGCCCTCATGACTTAACCACCTCCAAAAGGCCTCATCTCTTAATAGTGTCACACTGGTGATTAAGTTTCAACATGTATTTTGGGGGAACAT
TCAAACCATAGCAGCTCCTTCCTTCAGGGAACACTTCTTATCAGCCCTTCCCTAGGCCTGGAGACTCTTGCTCGATGTTTTGCCAGGCTGGTCT
CAATGACCTCAAACACTTACCATGCCACTTTCCTATTCCTCAGCTTATTCCCCACTCCTTCTGGTATAATATCATCTTTATCCTGGATGATTTT
TAAGTTTTCTCCAAGCCATTCTCTGCCWTAATTCTAGTACCTCCAGAAATCTATCTTGACCTTGAGGATTATTTTGAAGGCCCCACCTTCTCCC
ATTACCCCACACACTTAACCATTTAAGTGCACAAAAAAGAAGTAATTTAATTAATACATTTGCTAAATACTATCCATATTATATCTTATATGTT
GGCCTAAGTTATTGTCATCTTTTGACTTTAATACATAGTACTTCAGCTGAAACAGTCAATATTTTTCTTATGATGTTTGTGGGAACAAAGATAT
GCTCAATACAATCATGTTACAACATCCAGACTGAATGAAGATGCTTCACTCTTTCTTTGGGACTCAAACTAACATTAACGCTCTGCTGCTGGTT
ATTCATATACAGATCAGATTTGTAGCCATTGGCTGACTAGCATTACGAATTTAAGTCTCAGGGCATTATATTTCAGTGTACTATTTTCACATTT
ATCGCTCAAAAACAGGAATAAAATCACTTCTTCTTGTCCAAAAGATGTTCTGAAGAGTAGTGAATGGAAAAAAATTCCATCTATAATCTGATTT
TTTICACCAGTCTTATGACTTGAATATAAACTTAGAGATGCCTAACTTAAAAAATAACTACAACTTTCCTTATATTCATAATCTGGTAATTATT
CCACATTTCAGGCTTGATATATGATAAGGAATITTGCCATCTTTCCCAATGTTTCAGCCTGGATGGCTTAGATTCTTAGTCAACTAAATTATAA
TAATCTACTACTTTGAAACTATAATAAAACAATCATCTCATTACAAGAGTTACATTTCTTCTTGAAATTACTTTTCCATTTTGGGCCTTGTGGG
AAAAGCTGTTAAGAACTCTTCTGCAATTGTATAAATCTATACTTTGGGCTTAGATGTTGAGGCATTAGAAATGAATTCATCATTTATAGATCAT
ATATACCTTTAAAGATAGGCACCTTATTTCCTAAAGCAAGTAAATTAATTTTTTTTTCCACTCAATGCCTTTTATCAACAGGGGATCTTTTTTC
CCCCCTGAGCGCTTGAATGAGTTCCCTTTTTCTTCTGAGTTGGGGTATTACCATTCTATGGATAAATGAAGACAGAGACTGGATGAACAGATAT
AACAGATAGAGTCAGAATCAGAACTTAGAACTCTCAAACTCTGAGTTAATGAAAAATTTTCCCATAATTGTCTATGTTTAGGGAAAGGTAAGCT
TTCAGCCTTCCAGCTCCAGATAGATCAGGGTAGAACCTCTGGGAAGTTCTTTTCTGATTTGTATAAGAAATTACCCAGAACCCAAGTTTCCTGA
CCATAACCTGTTGATCTTCAAACATTTAAGAACCAAATGGTGTCCCCTAAGCACTCTGCTATTATCAGGAGGCTGAATTGACGTGTGTGTTGGT
GTGTGGATGGAGAGAGGTGTAAACCAAAAATAAAATTCTAAGCACCCCCCACGCCCCACCCCCTGCCCACCCCGCCTTCCCAGGCAACCCTCTG
AACGGACTAATTTGTCAACCAGGGCTCTTGTAAAAATTTATCCTGAAAGACTGGTTCAGCCCATGAGGGATGTGAGGGTGGGACGTGCCCCATT
CATTATATATCAACCAACGGACTAATTTCTCAACCCAGGCTCCTGTAAAATTTATCCTGAAAGACTGGTTCAGGCCATGAGGAGAAGTGAAGGT
GGGATGAGCCCTGTTATATATCAACATGGAGTTCAAGTCTGATAAGAAACATTTTACAACCTATTCTCTCTGAACCTGCTGCCTGGAGACTTCA
TCTGCATAATAAAACTTTGGTCCCCACAAGCTCTTATCTTAACCCATACGTTCCTTTCTTTGATCTCAGGTCTTTAGACAAACTCAACCAATTG
TCAACCAGAAAATGTTTAAATTTACCTATAGCCTGAAAGCCCACCCGCTCCTTAGAGTTGTCCAGCCTTTTTGGACCAAACCAATGTATTTCTT
AAATGTATTTGATTGATGTCTCATGCCTCCCTAAAATGTATAAAACCAAGCTGGACCCCAACCACCTTGGGCACGTGCTCAGGACCTCCTGAGG
GCTGTGTCACAGGCCATGGTCACTCATATTTGGCTCAGAATAAATCTCTTCAAATATTTTACAGAGTTTGACTATTTTCGTCAACAGACGTTAC
ACAAAATCCTCTCCTATGTAAGACTATCGGATGTGTACGTGATGCCCGTCGTAACACTGGTGTGAAAAGCTGGACTGGGGCGTATGTGCCTGCA
TGTAAGTGAAGGTGCCCAAGGGGATTTCTCTTCACAGCTCATTGAGCATAGCTGACTTGCTACCTATGGTCTGACACCTCCTGATTTCAGATCT
GCCCTGAAAACAATTACATGAATATGCCCCTGCTACTCAGTATACTACTAGCCTTGGAGTCTGAAGTAGACCTCCAAACCCCATCCAATCAGGT
AAGGGAGAAAAACGATAGCCCAACATTCCCTAGTGCTCATAATGTATTATTCCATTATTTCACTTTTTATCCCTTCTCTCACAGATAAGAAAAG
AAAAAGCTCTACTTCTTTAGGGTTCTTGGACGTAGACTCTCCACAGTTCATCTGCGGTTGTTCATCTCCACTTCTTTTTGTATCCCAGGGCAAC
TTTCCTGCAGTTTCCCTAATGAGAATATTTTCCGGACTAGGCAAGGGGCGGCTTGCCCCGCCCCCTACTCTTCTCCCTGAACAGGNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNGGAGAGGCGGCGTCACCTCCTCCACGCTCCTCCGGCCCCATTCAATCTAGCTATTTCCACTCGGCTCTGGCTCTTTTCCGCC
AGCTGCGGCTGTTCCAGAAGCGCCGGGCTGCCGGTCCTGGCCTCGGCAGGGGCCGTGCGGGACCGAGGGGTGGCGGCCGAGGCATGTGGGGCGG
GTGGGTTGCGCGCTGCTGGCACGGCGCGGGCACCCAGCGCGCCAGGCGGGAAAGCGGGGCGGCGGCGGGCGGGGACCCAGACCCGGAGGGGCAG
CGGGCGCGTAGCGGGCCCCAGGACGCCCGGCCAGGGGATTGGCCTCTGCACCGCTGCTCAGGGCGCGTCCCTTGCCGCCTCTGGCGTCGACTCA
AGCCTCCTCCTCCGAGCAGCCCTCAGTGGCGCCGCCTTAGTGCTCTGGGTCGAGCCCCGGGAGGGTTAGCCTGGGGACGGCAGGGGCGTGACCG
GGGCGCGGAAGTAGGTGAGCAGCAGGGCGCCGCGCGGCGGTCAGGCCGGGACAGCCAGAGGGGCAGCGACATCTGCGGGCGGGCGCTTCCCGGG
CGGAGCAGGGCCCTCCGGAGGACGCAGGCCGCGTCGAGGCTGAAGGGCCTCCCTGTCCGGGACGGTCAGCTGCCAAGTTCGGGCCCTTAGCGCG
TGGCCCCGGGCCGCGGCTCGGAGCGGAAGCGTCGCCCCTGGCTGCGCGCTCATGCTCCTCCTAGCGCAGAAGACCCGGCGAGGTCCCCGAATTT
TGGCTTAGCGTTTGTCAAGTCACCTCGAGCGACTCGGAGAGAACCCGGCTTTCCCACTAGGGACGGTGATCGCAGTCTCTTTCCTTTAGAAAGT
TACTCCGATACTTTTGATCATCTGTCCATAGTAAGGAGGAAAAATAGCGTGGTTTGTCCCCGGGTGAGGGAGTGTTGGAGGATCCCGTCCAGTG
GGAAGAGCCAGTTTTAGCGGAGCAGCCCGTCCAGCCCGCTGACACTTCAGCCTGGTAAAGGGTTAACTGCATCCTGCCGGTTCGGTTTACATGT
AAATAGAGAGCAGCTGCTCCGCGCGGCCCCGGCTGGCTCTTTTAAGTTTTTGATTGGTTGCCAATGACATCACCGCCCGTGTTTTAACACCGAG
GACCCAGAGCAGCTTCGGATAAACCCTCCTTAAAGAGATGAGGCCACGCCTTCTTACCCGCCCCCTAGGCTGCCCATTGGTCGCGGGGCTCTGT
GAGGTAGCGGTCGGCTTTCTCCGGATTCTTATTGGCCCAACCCGCCGTCGGTCGGCGGCGAGTCCGCCCCCTCAGAACCTTACATTTACAGTGT
GTGCTTGTGTGAGTGTGAGTGTGTGTGTTTGTGTGTCTGTATCTGTGTGTGTGAGAGAGAGAGAAGAGACAGGATAGATAGAGATGCGATGCTG
ACGGAGGAGCTACAGCAGGTAGGGTTATAAGGTTCCGGCCGTTGGCTCCTGCTACCCTGTGCGTTATACTCCCTGAGTCTACTTCGATTTGGGG
CGAAGAAAAAATGAGGTTTGTTGGGATGCTTTGCGGCAGTTTTAAATGCTCGGGGCTCATTAGTGCGCTGTGTGCCGAGGAGTGAAGCGGGGGG
CGGGGGGATGTTAAGTGCTCCAGGAATGGGGAAGAAGCATTCTCTAACCACTTAATTTTCTCTCCAGCTCTTGCCTAGTGGTGGAAGGGAGAGG
GGATAGCTCAGTATTTCTCTGGGAcATTTTCTGGGCCAGACTGGATGGTTTCACGGGCAGTATCTACGCCCGACCCTCTGCTGCAGCGATGCAG
GTCTCTCTATACACTCCGTGTGTGTGTGTGTGAGCCTTGACTCTGTGTGGAGGTGGGGGTCCCAGCGCGTGTCTCTGTACAACACGCTTATGCG
TCCCTCTCAGAAGAAATGGTATGTTGATCCTGGACGTGTCCTACCAGGTAACACAATAGCAAAGACTTTCACCGCTTGGTCCAACCGGGAAACA
GTTATAGCTTCTGGAATAGAACTCGCTCTTCAGAAAAAATACTACGCAAGGGAAGGGGGGTTGACTCCTTCGCTCCCATCCAAAGGTGGGTGAG
AGACCCGAGTCGTTGGAGATCTGCCGACGTGATGGGTGCGTGCCCCGTGTGCGCGCGTGAGTGTGGATTCCGCCTGTGAGACACTTTGGTTCTG
TGTCAGAGAATGCTGACGGTCGTGTGTAGACGGATGAGAATGGTGACGGTCGTGTGTAGACGGATGATAGATGGCATTTTTCCTGATAGCTCTT
AAAGTTCGTTTAGGTGAGCGTCTCTTGGCGTGGGGTTTGGCGTCTTTGCTATGTGCATGTCTAATAGAAGAAGAAGAGTTAGCAAGCCTGAGTT
GTAGAGTGCCCCTGTGCCTACAGGAGGAAGGAGCTGTCTTTTGCAGACACTCTGAAATCAGAGATAGATCCGGTGGGCAACAATTTCACTGTCC
CTTTTCAACGAGTACAGGCTAATTGGGAGGTCCTCTTTACGGAATGAGTACTCTTGAAGGGGGATATCTGTATGGGATTGAAACCTTATACAAC
GTGGCTGCCTCCCATTGAGAGAAGCTCTAGGATGCACAGTCTGTGCCAGCCAGGGAGATGAAGAGGGGAGGAGAGCCCAAGTTGTCCTTTCCAG
GCAGGAATGAGGGCGCCGGCAGGTGACAGAGCAAAGGGTGAGGGATTTGCCGGGGCTGGGGTTTCCAGGTTTCCAGATCTGGCTCCATGGCTTG
AGATTTCGTTTTAGGACACCAGTTAGTAGTGAAAAGGGGAAATGCAGATTAATTTATAAGAGGCCAAAAGACAACATGTGAGGTTTTAGTATGA
CTATGTTGTGCTTTTCAAAGGACCCCAATAGAGAATGGAAGGCAAGAATAGCTCCAAGTTAAAGAGATTCACTTAAGGGGATTTGCAGAAAGTT
GACATCCACCCTGATGTATATTTAGCTGCTGTACCTTTCTGTGAACCATGCCAGTGCTCTTGGTTACCCCTTTGCCTTTGCAGCGAGTGAGGGA
ATTAAATGGCAAGCATGCAAGGGTGGACAAATGGGTCTTTCTCAGATGTTTGCAGTGTACTTCTGCCCGAGAGCTGAGGTTCAGTAGATGGTCC
TCTTAAGAGCCTCCCTTCTGTCTTCAGCTTTTCTAATTATTTGTGCTTATACCTTCTCTACAGCTGAACTTTTATGTAGGTCCAGGAAAGGTTG
TTCATTAACATTTACAACATTACATCCAAATTTAACCTTAGACTTAGCAACCAAATTGATTGGAGCCCTCAGGTTTGCATCCTCTTTGAATACA
TAAAAATGAATTTCAAGTTTTGATAATGGTATGATAAAGGGCAATTAGGACGACTTGCTCAGAGGGATGCAAAGGTCTTTCCCAGGGATTTGGA
TGAGACTGCCTTTTGTGTCACGTGACTTTATTCTTTCTTTCCCCCTTCCCCTTCTTTACAGACTGTCTTGAGTTCTTCTTGGATTCCCAGTTTT
CAGCCTCCTCATGCCTCCGTCTCCTTTTAGACGACAGGGTAGAAGTGGCACTATCTAGGCCCGTCCGACCTCACCATCTCACCTTTGTTTACAC
TCTAGTTACCTTGGCTCTCCCAACCCAGGCAGTAACAGCCACCCTCCTGTCATCGCCACCACCGTTGTGTCCCTCATGGCTGCGAATCTGACGT
ATATGCCCTCATCCAGCGGCTCTGCCCGCTCGCTGAATTGTGGATGCAGCAGTGCCAGCTGCTGCACTGTGGCAACCTACGACAAGGACAATCA
GGCCCAGACCCAAGCCATTGCCGCTGGCACCACCACCACTGCCATCGGAACCTCTACCACCTGCCCTGCTAACCAGATGGTCAACAATAATGAG
AATACAGGCTCTCTAAGTCCATCAAGTGGGGTGGGCAGCCCTGTGTCAGGGACCCCCAAGCAGCTAGCCAGCATCAAAATAATCTACCCCAATG
ACTTGGCACAAGATGACCAAATGCAGCAAGAGTCACCTGCCGAGTCAGGGCCCTGTCATCATTGACTGCAGGCCCTTCATGGAGTACAACAACA
GAGTCACATCCAAGGAGCTGTCCACATTAACTGTGCCGATAAGATCAGCCGGCGGAGACTGCAGCAGGGCAAGATCACTGTCCTAGACTTGATT
TCCTGTAGGGAAGGCAAGGACTCTTTCAAGAGGATCTTTTCCAAAGAAATTATAGTTTATGATGAGAATACCAATGAGCCAAGCCGAGTGATGC
CCTCCCAGCCACTTCACATAGTCCTCGAGTCCCTGAAGAGAGAAGGCAAAGAACCTCTGGTGTTGAAAGGTAATGCCCCTGCTCCCTTCCCAAG
CACGGTCCTGATTAGCCGGGTTCACTTTGGGTTTTCTCGTAGAGTGACAGCATTCTCCTTACCCTGGCTACCAAAGACCAAAATAGACTTTATT
CTTCTCTTCCCTTCTATGCTCTTTATTGTTTTGGTTTAGTAAGAAAAACCCAGCAGCACTGAACTGGAAGCCAGAAGATCTGGGTTCTACCCTT
CATTTGGGTTCTAATTTGCATTGTGCAAGTCATTTCACCTCTCTGGGACTGTTTTCTCATCTTTCTAAAAAGGGGCTTTATACACACCAGATGG
TATCTCAAGTCATGCCTCGTCTGCTAGCTCTATTCCAATCAATCACTAGTTGGAAAGTTATTGTCTCTCAGACTTAACCAATGTTGATGGAACA
GAGGAAGGAGCAACTATGGTGAACTACAAGTAAACCTACAGGTACCAGTTCTTACTGTGGGAAATTCTTTCTCTTTCTCACCTTTCTGACCAGC
TTGACTTTTATGTGGTTTATAAAGTGACCTTGAAGAAGGGCAGAGCTTCTTATTCTAAGGCCCCATATAAAGTATAAGTTCACCAATTACATCC
ATTCCTCTGCCAAGGCTCCAAGGGAACAGAGACGTTTTTATGAAACACTCCCCAACCCATTGTTTTTTCTGGCCTCACCTGACAACGGTTGGCA
TCATGGCTCCTCCCAAGATGGAGTAATGGGTCACTTGCCCCAGTATACTGAATGCAGAATAACTGAGATTAGCAGAACTGAGCTCTGGCAGAAG
GAATTATTCACCACTCTTCTCGGAGCACCTTCCATACCGTGTTGCCTAACCTTTCCAAGTAGCCTGCATCCTGTGCCCAGCCTAGTTGCACCTC
TTTGCCCTCAGATACTGACTTTTGGGGCACTGTACATCACTCTCATTCGGCATCTTTTACCTGGTGGTGGTAGTGAGAGATAGTCGTCTTCTCT
CTTCTCTCCTCTTGTGTTCTGAGCCAAGGTACGCAGTTTCTGGGTCACATCTCCAACCAGTCTCCTAAAGTGTGTTAGACCAAGGTGTTGCTGA
GTTTGAACACATTGCAWATTTAAATTGCAATAGGACATGTGGGTGAGCAAGTTAGTTATTTTGTCTCTATGGGACAGCTTTGCCATTCTCCTTC
TTGTCTTCCCAGTGGGGCATTCTGCCTTTTGTTAAGCAGTGTTCTGGGAATTATAGTTCTGAATTCCACTGACACAATCTCCTCTTCCCACCCT
CCGCTCTCCTCAAACTTGCCCTCATGGGGCTACACTACCACAAAGGCACATCTCTCCTTAGGGCTGGTGGGCTTTGCTGGGAAGGAGAACATGA
AAGAATTGTATGTAGAGAGTTCCAGAAGCTTCTAGACATTTCCTGGTCGTGGTGGTTGTGGTGGGTTTTCTTGAGGTCACAAATATGTTCAGAT
TTCCTCCATATAGCTCTGTATTTTATTGTTTCTGTATTCTTGTATGGTTGACTTTGAGTGATTCCCAGGATATTTACCCTGCTATTGTCTTACC
ATAATCTTGATGTGTCTAGCCTCTTACAAATGAGAAAATCCTACCGAGGAATTTCCCATTTGTAATAGTTTGTCTTGGGGAGCAAAGAGGAGGA
TAGCCAATGATCATGATGATCTCCTCTTTTCTCACGCAGAGCAGGTGTTCATGATAAAAAGCAAGTCTTTTGTGTCTTCATGATCTGAAGAGAG
GAAACAACACGGATCAAGATGATCCTAGACCTCCTAATCATGGGGATGATTACAAGGAGTTGCAGACCCTACTCTCTATTTTGCAGTGGGTGGA
GTTGTGGATAGGGGTGACCCATCAGTCATTGAGAAATGAATCTCCTGGGTGCTACTTGTGAAAGTGAGGATCCTCTCTCCTTTCCTTGTGGATG
CAGCTGAGATTCTGGACAGCCTAGACTTCTTCCTCAGTGTCAGAGTAAGATTGCCTCAATGGGGCGGGGCAGGACACACTGCTGACCTCACCAC
TGGGCGCCCTGGGCAGCCACTGCGAGGCCTCGTCATTTTCCAGTTGGTGAGACACCGCGGCAGTCGACTTGGAAGATGCTCTGGTGGTAGAGGT
TAGATTATTTGTAGTTTTGGGGGCCTTTTCTTCAAATATCTGGGAATTTCATTGAAAATATGTAGTTCAGGAGTGAAGGGGTCTATATTCTACT
TTATAATTATCATTATCATTTTGGTATCTTTTGGGAGAATGGCCTAAAATTGGGACCAAGATGGAAAAGAAGGGAAAGGAATGGGTCAATATTA
GAATGCCCTTCGTTATCCTTTCAACTTGGTAAACCAGAGATTAATACCATAGCACCACTGTGGTGAAAGACGGGGTGGGCATTCCCCAAAGGCA
TGACCAGCAGACTTCTCTCAGATTCTTCAGTGGGTCAAGCAGACCATTCTGGTCTGGCTTCTCCTCTCAGATTTATTTGAGTGTTCCCCTCCCT
TGGTGATAAGATTGTGTTTCAGCAAGAGTGGTAGTGGTACCATTTCGGAAGGAGTAATTTTTGCAGGGAAGTTGGTGGGAGGGGGGCATAGTTA
GAACATAAGGAGGAAACTTCTAAAGGCTTGTTAGTTTATTTTCAGAGATTTCAGTACAATCTTGGGTAATTTATGGCTGGCCCATCTGATGCTC
TGGTTTATGTTTGTAAACACTGGTTGCTACCCTCCCTGTCCAAGATCCTTGTCCCCCTGTCTCCATGCCATACTCTCCATTTTTGATGCTGTCT
GTCAAGTAGAGCGGCTGCTAACTTGCCATTTTAGTAATGCTGTGTCAGGTTGGGAAGAAACCGATCAGAGGCATGGCAGCCCCTGGGATTAAGT
TACTGTTCTAATTTACAGTGCCCTCCCTGTTGTGACCTATGCTAGAATCTATGTTTTAGTCTCATCTTCTAAAGAATAAATGGCATCAGACCTG
TGCTTCTCTTCAACCTGGTGAAAATGATCCAAATGAATAAATATTTAGGGATTTGTGAGGTCTTTGAAAGAATTATATTGTTTACTCTAAAGCT
GATTTTTAATCAACTAAAACTTGCTTTCTAGTCAAACTGAGTAAGAAAGCTGAACAATTCTAGACCATCCATAACGAGAGCTACCGTTTTTGCT
ATAAAGTTTTTCAGGCTCCCTTTCTTTTCAGTAAAGCTGGGTCCAGGGAAATAATTTGTCATTTACTTAGGTAGGAAGAAATAGTCTACTGCCC
CTTTTTCTTAAAGACAGTATATGCCTTCAAAAATGTTATTATAAACATTTTCAAGCATATAGAAAAGCAGAAAAGTGTGTAATGAACATCCATA
CACCTATCAACCAGATTAAACAATTATATTTTTCCTATTTGCTTTCTCTTTTCTGTTGAAATATTTAAAAATATAGATAATGTTTTAATGTATT
TAAGTATGTTTAATGTGTAAATTAATGTTTCAAATTCAATAAAGCTGGAAATATTTTATAGTATACTATGAACATTTTCACTCGTAAACACTTG
AGTGTCATGTACCCATTTTTAAGTGCTTTTCAACTTCGAATCCCTCTGTGACTGACTGGCAGTCAGGTCTACAGAGGAGCTTCCCTCCCTGGGA
GTAGGAGGGCTCCAGGTGCCTCCTTGCCGTCTGTGTGTCCCCCTTCCTCCCTGGTATGTCAGAGCCCATGCCTCTACTTGCTCTATGCTGTTGT
AAAACAAGGAATGACCAACTGCTACAATCTTTATAGGACTGCTCTGTACCAAAGGCCTTTACTGGGGGAATGTGAGAAGAAGAGGGAAATGGTT
TAAAGGATGATCCATGGGGAGGATTTGATCATGCAGGCAGTTAGGCCTTTATACTATAAAACTTCATCACTCCTTAGAATTTGAGCACCATACA
TTGCACACTTTTCCCTAGTTCAAAAGAATTCTGAAATATACTCTTATTGGCAATAGAGTTCTATGACAAAGTGCATGGCTAGGCTCAGAGTTCA
GAGACCTGTGAAAAATATTCTGTTTCTGCCCTTTTTTCGGGGGTCGGGTGGGGAACGGAATAAAAGTCTCCCCTTTTCTCTCAGAGATGTTATG
AGGATTTTAAAAAATACCTGAAATAAAGACATATCTACATTCATAAACGTGCTGTTGTTATTCTGAGCCTCTGGTTAACATCCAAGGCTAAAAT
GTCAAACATTAGCCTTGTAGCAAATGATCCAGAAATCAAGGCAGAACTTCCGATCTAGTCAGCCTGAATGCTAGCCTTGGCTCCCCTCTGTCTA
GGTATGTGCCCTTGGGGAAGGAACTGAATCCTTCAGTTCCTTAGTTACCTCATTAACAAAAAGAGGATATAACAATAGCTATCTCAAAGTGTTA
GTTTCTGAAATAAATGAATTAATGTGTGTAAAGTGTTCAGAACTTTATACACATACATGGTAAGCACTCCATAAATATGGTAGAATGTTTTTAT
TGATATTGATTGAAAGGAAATGATAGTGCCAGTACAAAGGCAAATTTTGTTCCTAAGGAGTACAAATAACAACCTAGGTATAGTAGAGTGTGTG
TGTTGCTATGGCCTAAAAATAACTTGGTGACTGTTAACTAATTTAAGTTGCATAGAGTTGGAGTGCCAAGTAATTGCAAAACATATTTGGGCCA
GGAGGCTGGAGTAATTTGGGTATATCCAGAATTATTTGTTTTAGGGAAATTTGTAAAAACATGATAATAATTCTGTCCAAATAATGTCCCATAA
CAAAAGCATAAAACTTTTAGCTTTGCTTTTTTGTTGCCTCTCATCATCTCATCAGGAGGATTTATTTTTGGAATAAGGATTGTGGACTGACTAT
AAAAGGGTAGATGGAAGGAGGGGTGCGCTGGTTCCTGAGCCTAGGGTTGTGTTGAGTTAGAATGTGAAGGAAAAAGGAGGGAAGGAGTTNNNNN
NNNNNNNNNNNNNNNNNNNNNNNNNNNNNNNNNNNNNNNNNNTTCTTGCTTTTCTGTTCCTCATTCACGATGTGCTCAGCAGAGTGAAATTTTG
CTCACTTACACCTGGGTTCAAATCTAGGCATTTCCATTGACCAACTGTATGCCTTTAGGCAAATCAGGCGGCCTCTCAGAGGCCACGTTTTCTT
CATTGTAAACAGGGACAGCTCCAGCATCCTAGGTTGAACAAGGTGATACAAAGTACCTGGCATTTGATGGCTGTCATTATAAATCAAGCCTCTG
GACCCTGCTCTCCTACTCTCATGCACTAATGTAAGAACCTCTTACATTGCAGGGGCAGGGGTATTGGGGAGGATGGGCAAGTCGTTCTAAGCAC
AGTACTGTGGGAGCCTGTTAGTATGAATACTCTTGCCCTTACCAATTCAGAACAGTTAATTAACTTAATCATCCAGCTTGTCATGGACATGGTT
CAAGAAACTCCTCTCCTGGCAATAGGAGACATGATTTCACATTAGAAAAGCAAAGCAGGGGAAGGAAACTGAATACGTAAAGCACTTTGCCTAT
TTCTGTTTTCAAAGCACGTGTGTATAGGAGAAAGGAGAAGGGAGAATGGTGAATATGTACACTTTTCTACTTGGTCCTTTGGGCAAACCCATCT
GAAGGTCTGACAAAATTACAGGAGCAGAAAAACTTTCCGTCACATACTCCTGGTGCATACACCCTCAGGGACTAAGTGGGAGCTTTTGACCAGT
AGCAAAAATCTAGTTCCTTAAACCCTCCATTATCAGAAGCCAGAACCTCCAAGCCCCAGCATTCCTCAAGAGCAGTTTGGGCATGAATGTGTGA
TATATCGGAGTGCTTAGGCAGTTTAATCCAAGTTGGTTGAAAAAGATAGACTACATATTAAGGAGAGAAATTCTAGTTCCAGGAGAGGTCATCT
CACCTTTTTGGAACTAAAGAATCCCAGCTTTGAGAAGAGCAAGGAACTTTAGAGGGCCCCTAATCCAGTAGTTTTCACTGCAGGCTGTTCATTG
GTATCACCTGAAGGTGATTTATAACCTATGATGCCCAGTTCGTGTGTCCAGAAAGCCTGATTAAATTGGTCTGGGGTGCAGCCTGCACATCAAG
ATTTTTTTAAAGCTCCCAAAGTCATTCTAACAGGCAGCAAAGTTTGAGAACCATTGATCTAGTCCGGTATTCTCATTATGCCACTACAGGAATG
GGGCTTGTCCAAGGTCACATGCATGATTAATTTTAGAGATTTAGTCCTTAATGTTAAAGTAGGTCTTGCCAGGTTCAACAGATTCAATAGATAT
CAACAGACTGAGGTATTCGTATGTATCATTATTTAATAACAACAGAATCTAATATATAGCATAAAATATAAGATTTTTTGAGTACTGATGCCTT
TTTTCTTGAATTTTGAGATTTCAGTTCAGAACTATTAACACCAAAGTCTGAGGACTTTCAAAACCAGCAGTTCATGGTCTATATATTTACCTGG
ATATTAGAATATGGACCAAATATTAATGTTCATTTAATCTCTTCATCTGGGGAACCTAGTGGTTTAAGCTGTTAACCTACTGGTTTCAGCTGTT
AACAGCTATCAAAACAGGTATCAAAGAAGCCCAACCATCTTAGGCTTCTAGGGACTTTTTGGTAGGTGGTCTCCACAGCTGTACAAACTTGTGG
GCAGAGGAGATTCATTTGTTTGTTCAATAAATATTGTTGAATGCCTTPGGTGTGCTAGGCACTAATCTGCAGGCTGGGCAATCTGCATCACAAT
GCTTATCATTCAAATGCAAGAAAGACAACATATATGGATGGGACCAGTCTTCCAGATCCTTTCCATCTTGGAAATTGTGTTCATGTTTCTATTC
TTCATCTTCTTCTGCCACACAATCTTATTTAGGGAGTGTTTCATCTTCACGAAATTTTGTTCTCCTGTGAAGCACATGTTTGTACTTTTTTTTT
CTTTTTCTTTTTTAAGAAATAGCCAGCCGGGTGTGGTGGCTCATGCCTGTAATCCCAGCACTTTGGGAGGCCGAGGCAGATGGATCATGAGGTC
AAGAGATTGAGACCATCCTGGCCAACATGGTGAAACCCCGTCTCTACTAAAAATAcAAAAATTAGCTGGGCATGGTGGTGCACACCTGTACTCC
CAGCTACTCGGGAGGCTGAGGCAGGAGAATTGCTTGAACCCGGGAGGTGGAGGTTGGAGTGAGCCGAGATCCCGCCACTGCACTCCAGCCTGGC
GACAGTGAGACTCTGTCGCAAAAAAGAAAAAAAAAAAAAAAAAAAAGGAAATAGCCTAGCCTTTTCTAAATATGTCATGGGCCAATAGGATTTC
TTTCCAATGTCTTGATCTGAAAAGAAATGATTCATTGGTTTTTAATGAAAAAATAAATATATATGCCCTCTCTGCCTCAAAAGCTCAGGGCTGA
CAAGTCTCCTCTGTGTCGGAGAGTGAAACTTTGTAATTTGGACCCCCTAATTTTGTACATGTTCATGATAGGAATAAGGGCTTCGTTTATTTTC
ACTGCATGCTCTCTATGGAAAGAGGATGTGCTAAGCAAACAAGCATTGTAAACAATATTTCAGAGGCAAGGTTTTGGCCTGCTTTAAAAAAAAA
AAATGTTTGCAAGTACAATTAAAAACCAGTATAAGGGACAGGGGTGGGATGAAAACCTGTCTCTAAGATTACGAAGCCTGCGTTATTTCCCCTA
AATCCCCTTCGAGCAACATTTGAATCCCTCATCAACAAATTTTCATTGATTATGTTTCTATTATATATACTGTAGACTCTATATTCACGAATCT
AATCATACTCATTCAGAAAAATATAGGAAGAGAAATGAGTATGACCTGTAGCCTGAATTTCATTATAAAAAAGATTAAAAATATACATTTTATA
TTAAAATTGATGTAATCTTTTAATTATGAAGTCTTTGATTCTTTAGATGTTTTCATCCATAACCCAAGAGCAAGATCTTGGCATCAGTTTTTTC
CAGGTTATGTCTATATCATCTATTATTACTTAAAAGTTGGAGTTACATATAGATATATAGATATATAGACAGTTATAGATATATGTAATTTTTT
TTCATTCCAGTCCCCCACCCGAGCAAGACCCATATTTTATGTAACTTAAAAGTAAAGAAAAGAAACTTTGAGCGTATGTTTTTGGATAAGGAAA
TGAGCTGTGCAGCCCTAGTACTTTCATATAAGATAAAGGCTCTTACCTATTCTCTGAATCTGGCAAAGTAGAGAATCACTTACAGAAAGAAAAG
GATTTGTGGATGCATCAAATTATCTGCATTGCCCTTAATGTTTTATGTGTGGTAAAGGCCTACTCAGGCCACCGCCCCCAACCCTGGCCCCCGA
CTTGAATTTCATAGAACACAAAACAAAGCGCAACTCTTCCTGAGTGCTTCCACAACGGGATTTTGCTTTCTTGCCTTACCACTTCAGGATGGAC
CACCATCAGGCATCTGTTGTCCATTGCACACATCAAGTAAACAGATTAAACTGCCCGAGCCAGGGGGTCGCATCAGCCCTGTCCTCCCTTTGAG
GAGCTTAAACCTCTCGTGTTATAATGCGAACTCGACTGATCTTATTTTGTGTCCTGAGGTAAGGAGGCACTCACCGTTTTGCATCAGAACATCA
CTATCCGGTTCCTTAATATCTTATATTTATTTTATGGAAGAAATAATCTCACATTCTTTGTATCACAGCCCTGAGCTTCACTACCATTGAGCTA
AATATTACCAGCTGTTGCTACTGGCAAGAGAGAAGCAAAAAACAAATTGAAAGGAGAAAGAGGAGGGGTGGGGGAGTGGACAGAAAAGAATAAC
ATGAGGGTTGAGGGTCTTAGGGTGACAGCAACATATCTTCTAGCTCCCAGACCTGAGTTCTGGGCTCCAGTATTQAAGACTGTGAGTCATTTTC
TTCAATCATTACAGTGTTTCTTCCTGTCTTAAAAGAATAACACTAGACACAAACTTCTAAGAGGGCCTTCCCTGAAGAGATAGACGACATTTTC
TATTGTTTCTTTTAATTTCTATTTTTATTAAAATCCTATGTTTTTATGATAAGACTTTAAAGGAACGGGTCTTATGGTTCTCATGAGGTGATAT
TTTAATCTGCCAGCAGAACATCAGTTAATCACATAGAACTACTCTGGCTCTGTGACCAACCTATCATCCCTCAAATTAGAAAATTATTTTCCAG
ATAGTTTGGAACAAAAACAAGTTAAAAGCTATTGAATGCTTTCTAGATGCTGGGCATTGGGCTGTCATATATGTGTCTTATTTAGTTCTCAGAG
AAGCTCTGTGAACGTCATTTTATATCCTGACTTTTATAGCTCAGGAATCTGAGGAACAGATTAAATATTTTCTCCCTTGTTATTAACTAGTAAA
TGAGAGAATAAGGATTGAAATCCAGTTCTTATGATTTCAAAGCCCAAATTCTTTTCAGGCTGGCCATATCTATTTGCAAGTCTCATTCCTTATG
GTGGCTTTAGTTGTGCTTTACCTCTGTGGTGCCTTAGTTATAATGCCATCTTCCTTTCCCCATTTTTCTTGTATATCCTGATAAAGATATTCCA
TTGCTAAGGTGAGTAGCCTATTCTGTGTCCTTAGAGTAGACTGGCTTCCCAGTAACCTTGCCTCAAATTATATCCCAGCTTTTGGATACTTTGA
ATTTTGGTCATGACAGTTTTTTAAGGAATAAAAATTTGAGCAAAACAAACTTTTGAAAACTGTGATAATTCTAACATATATGAAGTATTTCTTA
CATCCCAGACTCTATGCCACAAGTGTTATGTCTCATGATCCCTTTCCAATTTACATACGAGGGACTGAAACTCGGAGAGGTTCAGACACTTCTC
CAGTGTTACTCAGCTGGTGCCTAGTGGATCCTGGGTGTAGCTCTGGTATACTTCTGCTCAGTTTGCTTTTGATATCTGGAACCTTAGAATTCAT
TCTCCTAGTAGCCTGTGGATAGAAGGATGATATTTCCTGCTTTTGCACACATTAATAAACCCACTTTCTGGAGTAGGAAGTAGATGAACAAAGG
TAAAGAAAAGTACAAGTACAAAGTTAGGTAGAAAAGAACAAGAGAATCTGTGACGAGTATGATCACTACATCCTAAAGACTCTAATGCCAAATG
GTATTTGATTGTTTATGCTTTATATTCATTATCTGTCCTATTAGTGTGGATGATAATGAAAACTATAGATTGTTTTCCCTCAAGTAGGTGAAGA
AAGAATCTCTAGTAGGTTATGATTGCCCTTTGGGAAGGTTAGTTATCAGGGATAAGTGAACAGATCCTATCAAACCATTTACTGTTTAGTTTCT
CTCCTCTCCTTTCCTAATCTTTCTTATCTCTGTTCTTCCCTTCTACAGTCTAAGCCTACAAATATAGCTCAGGTAATCTGGAGGTGTGGCAAGG
ACAGACCCTGGGAAAATAATAATGAACACATTTGAAGGACAGCAGGATGGACTCCTTGGAATCCTGTGTTAGCAGGCAGCATTCATACAGCCAC
AATCTGTCTTCTGACAGAGCACCCAGCTTCACACAGTGAAAGTATGTGACTGTCCTCTCAGATATCAACTATAAGGTTTCGTCACTAAGTTCAT
TTTATGCTGCTAAAACAGAATACCTGAGATTGAGTAATTTACAAAGAACCATAGGTTATTTGGCTTATGGTTCTGGAAGCTGTGAAGTGCAAGA
GCAGGGTGCCAACATCTGGCAAGGTCAGCCATGGTGGAGGGTATCACATGATGAGCAAGTGTGTAAGACAGTGAGAAAATGGAGGCTAAGTAGT
TCATCCTTTTTATTACGACCCACCCCTGTGATGATCCACTCCATGGAGATCGCCATGCTATTATGGTCTTATCCACTCTTGAGAGTGGAGCCCT
CATGACCTAATCTCCTCTTAAAGGCCCCATCTTTTAATACTGCTACAGTGACAATTAAGTTTCCAATACATGAACTTTGGGGACATATTCAAAA
CCATAGCAGTAAGGTCACCCCAAACACTCAGGTCCTCCAGTGAGAGGGGAAGGGAGGGGGACATGGGTGATCAAGGCAGTAGAATTAGTGAATG
GAGAACAAAATTTAAAAATCTGAGCATTTTTTATTCTGAAATATAAAAGTAATCAATTTTAAGAAAGCTTAAAATAAATTCAAGGAGATTAACT
TCACTGATCATAAGTTCTCACATTTACATGGCAAGGTGAGTTTTCTGAACCACAACAACCCCAGGATGACTTTAGAAATGTTGAAAAACCACAG
AATTATAGTCCATTGCACAGCAAGCATTCACTTACTCTATGTTGAATTAGAAAGGAAAGACATTTTTCAAAGAACATGGTGTTACAGAATTGTG
AGAGATGTTATCAAGCATTTGATCTAACCATCCCATTTTACAGACATAGAAACTGATTACTTATCCAGGAGGATATAAATAGCTATTAGCAGTA
CTGGCATAGGATTCATGTTTCCTAACTCTCAGTTTAGGGTGTTTAGAAGCCCAACCAGTATATAATCCTTTGGATTAGTATCTGAGTTGTTGTT
AAATAAGCAAATCTTGTGGCTTCAAGTATTCAGCCAGGTCATTCCATAATTTTTCTCATTACCTTTCTTAGGAGCGTATATAGTGACTGATTCA
ATGAGCCGGCTGATGCTCCCTTATATTTGTATTCAGTTTATATATTTTCTTGCGATAGGGAGTATAGTTTGGAGGTTAGGGCACAGATTCTGCA
ACCAGGCTGCTGAGTTTGAATTCTGATTTTGCCAATTATTATAATTTTGGACATTCACTTTTCTGTGCCTCTGTTTGCTTGTCTATAACTACTG
GGGAGGGTAATGGCTCTTTTCTCATGGGCTCAATTGAGAATTAGATAACATATGTAGAAGTGCTTTGAACAATAACGAATGCATACTAGCTAAC
ATTTATTGAAATCAACCATAATTATAATATTGAATCTCTACAATAATAATTCATAAATCTGCTTATATTCAGCTGATATATGAGTGGTATAGTT
CAGTAAAAATGCTTTGATGTCACCACTAATGCAGTACAAGTAGGAGATACTTTTCTGATGCTTGGTAGATCTTTTTGTTTAAAAGTTTGGAAAA
CCCAGTGTTTCTTTTGGGGTGGATAGATAACTACATAGGGTTTTTTTTTTTTTTTTTCCTGATAGTGCTGTGCTCATGATAATTATATGCTATT
TCTATGTAAGGAAATCTTATAAAAGTCTTAGCAGAAACACTTTATGTATACTTATTGTTAAGATATTAAGAACATTTTAGAAAGAAAAACATAA
TATTACTAATCCCGCCCTGACTGGCCTAGTTTCATTTTTACATTTCTTTTTGTTCTCTACCAGTATATGTGCATGTTTTTCAGGCTTTCTAGCA
TAGTAACTGAAATTCATTTTCACTTTATATTGCATGTACTTTTGCTTCCTGTGTTGTGATCATAGATTTTCTTCTTTAATGCTGTAACCTGTCT
TATTACCACATTTTACTTACTTGCCTATTAATATAAATTAAGATTGTTTCCAAGGTTTTGGTATGACAAGAGAAACCTCTTGAAGGAAACCCTT
GTTTGGAATTTTCTCCACCTTCCCACCCAACTGCACTCCCCATTTAGAGAAATGCGTACTAGCTGCCATTTTGGGTGCTACTTGCCAGTTTGAG
TTTATTTTTCCCCCCAATTCTCTTTCACTTTTTAACTTCTGCTTTTCTTTCCTGAGTGTAAGATCCAAATACCTATCAAAGTTGTGAGTTTTTA
GGGACATCTTTATCCTTGACCACAGGTCTTGATACTGATCCTGAATGTGTGTGTTTACCACATGTCCTGTAAGGCACATATTGCAGATCTTGGA
CCTCTTTGGTGGATAGAGTGAGGACATGAGGAAGCACCACTCAGCGCTATTAAAAATCAGAAGCTATTGTGGAAGTGAATGATCTGGCAAGGCT
AACAACGGCTCCTTATTTCATAAATATGAGTCACTCCCAGTTTTACACTGTAGATAAAACACTCTGAAAGGGACAATAAAAGCAGCAAGAGGTA
GATTTAGTGAGATGCTGAGTAGATGTGTTTGTGTCTCCCTTCCTCTCAACTTTTCTAAATCCTGAGGTTTTAGTGCAGTGCTGAAGTCTTCTAG
GGGCTTAAGCAAACACAGGCAGCAAAAACACATCAGAATGGAAAGATAATTCCCATTAGTTCATTTTGCCAAATTGAGGTAAAGTGCCCTTTAA
ATAGGAGATAATTCTTTGACAGATAATTAGCTTGTCTTCCTTGATAAAATATGTGAATTGGTTGAGTATATTATAATTAATTTTGCATATTATG
TAAAAATCTATTCATGTACACTTACTAGGTGAATCTCTTAATTCCTACCTTGAGGATGGCTAGAAAGCCCCATTTCTGCTCCAGTGAATAGAAA
GAACTGGAGGAATGCTGTGGTTATGGAGTCCAGGAAGCCCTCTCTTACAAAGATTGGGAGGAGCTGAATGTGGTATATCATTATTAAAAGACAC
AGAGACTTCATATTAATATATGCATTTATTAAAACAAACTTTGAGAACACTTTTAGGCCTGAGAATGAGATGCTACAAAGTTGCCTAAATAATT
ACCTTGAAATCAATCTCTTGCTAGTGACATTCTTCTGGCTTCCCAATGTGGAGCTTTTTCCAGGGAGTGGAGTGAGTGATTGGCTGGGTGCAGC
TAGCTGTTGGCTGGGCGGTGGCTGTGGGAAGTAGCCTGAGAGGGCACAGAACAGTTGCTCAATGACTCAGTGAGTCAAAGCTCCTCTCTGCAGT
TTTTCTTCCCCCTACTCCTTAAZCCATTCTGAGCTCCTTCAGGCATCCCCATGTATCTGCGCACTGCTTTTCGGGTGTCTTTTCTCATTCAAAG
CTATTGTCGTGGCTACTTGATTTGGCTCATATTAAAACGTGCACTGGGAGATGTTGGAGTGCTGAAATAAAAGAGCATGCTGTAGTTAATCGAT
CAGAGAGCCTCTCTTCTCCAAGTTGAAGTGTTTCTTTTCCTGAAAATAGAGTGGCTATTTGATGTTCCATGGGAGCCGAATGACTTCCTGGTGT
AGTGTGGCTTATAGCACCGTAGATTTTAGTCATTGGTCACTACCCCTCCATGTGCATGCACACACATTCACATGCAAACAAACACATATTTTAG
TTAACATTTGTAGAATGGGAACAATTATGACACTGAGCATTAACTCATTTAATGGGGATAGCAATAGTATTAAATCTATCTGCAGCGTCCAATA
TAGTAGCCTTGCTGCAAGCCAAACGTGGCCATTGAGCACTTGAAATGGGGCCAGTCCAAACAGAGATGTGCTGTAGGTGTAAAATATATACCAG
ATTTCAAGACTTTATATAGAAAATAATGTAAAATATTTCATTAGTAATTGTTTTAATATTGACTCTTGAAATGATATTTGCACATGTTAAGAAA
AATATATTAGCAATATTTTACCTGTTTGTTTTTACTTTCTTGAATGCAGCTACTAGAAAAAAAAAATGCTTGTTATTATAGTAGCCTGAATTCC
ATGTCTATCAAATGTTGCCGATCTCAATGTGGTAATAACAGTATTATTATCCCCACTTTTTAGATGAGGCAATTGAGGCTGAGAGAGGTTAGGT
AAGTTGCCCAAGGTCTCACAGCACAATGCCAAATTCAGACCTGCCTGACTCCAGAGCCCACACCCTTCATCACTAAGAAACTGTTGTATAATAG
AGAATTTGTCTGGCCGTTGTCCTCAGTTCCTGGGAAATAATTTCTAAACCCTTGGAACTCCCCGAGTGTTAGGAGTGTCTTTGTTATGCATGAT
GAGCCCCTTGGATCACACCTGTGTTTATGGTAATGCGATGACTCAGGATAAGAGTTTATCATCAGAAAGACATCCACATGATTAGAAGGTCAGG
GCTTTTGTATTAGTTCATCTTCTTCAGCAGAAGAGAGTGGGACTGAAGATTGAGTTCAATCACAAGGCCAATAATTAATTATGCCTAAGTAATG
GAACCCAATAAAAACTCTGGACACCAAAGCTCAGTTAAACTTCCTAGTGAACACATTGCTGTGTCAGCAGGGCAATGTGTCCTGATTGCACAAG
GAGAGGGCCTAGAAGCTCCATATGCAGGACCCTCCCAGATATTGCCCTGTATGTCTCTTTATCTGGCTCTTCCTGATTTGTAGCTTTTATAATA
AAAACTATCATCATAAAGATAGCGCTTGTGTTCTGTGAGTCATTCTAGTGAATTATCAAGCCTGAAGCATTGTAGAAGCCACAGAATTTATAAC
CAGTTGGCTAGAAGTGTGGGTGGCCTGGGAACCCCTGAACCTGTGGCTAGCATCTGGAAGTGAAGGCATTCTTATTTGAGACTGTGGCCTTAAC
TTGGGGAGTCTGCACTGACTCTGTTAGTCAGTTTCAGAATTATATTGCAGTATTGCAAGAATAGACTGCCTTTTATTTGGATGTATGATATTAG
GAGAACAGCAGCTGAGAGAGCTAAGTGCTGCAGCTTAACTTGCTTATCTCTGTAACACCATTTCCTTGGTGACAATGGCCATTTGCATTCTAGA
CTTGGAAATGACTCAATCTTTGAGCAACTGCTTTTGACCACTACGATCAGTTGGGAGGCAGGCACGCCTCTGAGACCTTCATACTCAAAACCCC
TTCACGCTGTAATCTCTGGGGCCTATTTCATCTCCCAGTTGGCATCATGTTAGCGGTGGTGACAACATATGTGTTGGCCCCTCTTTGAAAGCCA
GGGATTCTTAAGTATTGGTTTTGGGATTGAGATGTTCATGGGAAGCTGACACAGGGTGATACTATGGGTAAGGAGTATTTCGATTTTTTGTTGT
GTTTTTTAATCATTTGCCTTGTCTGGAATTACGCTTTTGTGGTATGTCCTATTACATTTCATAAACAAGGTATTTAGCTCAATTGTTGCTTTTT
CTTTTGGGGCTTTTGTTTGGATTTTTAAGCCAAGATCCATGTGTTTTATTTATTTTGGTTTAGTTTCTTCATCCCTGCTTAAAATAGACACTGC
CTCTTGTCTGTGGTCCTGGTATCCACTACTCTTCTGTTTCCTGCTCAGGCCTGGCCCATGTTCTGCAAGGCCCTCTTAGGTCACCAGCTCCTAG
AAAGTTGACTCCTATGAGTATCTCTTGGGGCCACCTTAGAATGCAGATTTCTAAGAAAAGTCCAACATAAACTCTGCAGTTATTGGGAGAAAGT
TTCCGTCACCAGTGAAAACTAGGAATAAAACAAGATTTTGGAATGTATAAAAGAAGCCTCTTTCAAAAAGAAATCAATATGACTTGTTTTTTCC
TAACATGGGCCAGCCCTGGGCACTTTTCATCTGCCACCACACCAGGCCTGCTCTTCAGAGGTGTTCAGCTTTAAGACTAGGGACAGTTCACACC
AGATAACTATGAGGCAGTGTTCTCTGCCACTCATTGACTGTGGAAGTCTCAGTCCCAGTTTCTGAGAGGGGGAGAGCAAATTCATCAGAACCAA
CTGAAGAGGTACAGAACATGAGGCATTCACTTCTCACACAGTCCCTGAGCATCTCACCTTCCAGAATACCAGAGGCTTCAAGGCCCTTTTTCTA
CCTTTCAAGACCGTTATAGCGTGCTATTGCTCTTTGAGAGATCTAGGGCTGTAACAGCCCTTATTTTTAGAAAAGAGAGAACTCCTGTGTTTGA
AGAAATAGCTCTTATGATATGATCGTGTGGTTTCACATGGTCTTAAGTTCAAGTAACTTGCCCAAAGGTGCACAGTTATTAAGAGGGATAAGAG
GAACTCCAAATTACTGATTTCAGATCCAGTGTTGTATCCCTTCAAAAGGAGATAGAAGAAACAGGGCCACCCGGGGTTGGTGAAGATACTGTCA
GAATCATCTGTTACTGCTTACCACATCTAAGTGAGAGGGTCAGCAAGCTCTTGAGAGZGCCCACCTAGATCTTAGCTTGTGTATACCCTATACT
GAGCACTGTGCCCCCATTTCATATTTAGTGCTTAATAAATGTATATTAAACTGAATTGAGGGCCCCTCCTGCTGCTCAGTGACATCCCTCAGGG
TGAGTTTCCCTCCTGAGCCTTCTTTTCCCTTTCATGACTACCTTCCATGCATCTCTACCATTCATCACTCATATAGAATGTCCAAGAAGTAGGG
GCGAGGAGAGAGGAGAAAGGGGTAAGAGGCCACCAAGTGTGATTTGCCTTGGATTTCAGCCCTCCCTGGAAGGTGCCCATTTGTCACATTTAAG
GGAGGCCCAGCACCCCTGTAAATTGCCTCTTCCTCATAAGTGTACTCAGCCCTCCTTCCCAGCCTTGGAATGCCAGGATTTCTAAATCCTTGTG
CTGAATTCACCTGGCGAGTCCTGCACCTGACTGTGTGAGTTACAGCTGGATGGCATTGTTCAGCTCAATCTTCAGTGTGTTCTGAAAATGTTTC
TTCTTCCAGTCCTGTCACTGAATTTCCCTAAAGTTCCTTCCTCCTTTCCCTCATTAGATTTCTCTTTGGGGACTATTTTTTTCAATCAAATCTC
GCAAATCCATAGCATAATGCTGAACAGCATATAATAGATGCTTTACAATAAACTGGTCCTATCTCATCTCATTCCAGTGTTCAATGAATCTAAA
AATATTTAATGAGCACTTACTATATACAATTCAGATGCTAAACAGTGAATACTTAGAATTTCTATTAATAACTACAATGTAGTAGCTGATTTAA
TTCTTGCAATAACTATTGTTGCCTGTTAGGACTATTATTAGCTCCATTTCAGGGTACAAAGAGGTGAAACAGCTTTGTCAATATCATACTGGAA
AGTTGTGGAGACAGTCTGGCCCTAGTGCACCAAACCATTACACTCTATTTCCTCTAAGAGAAAGAACATCTTGACTTTCTTGGGGTCCACACTT
TAATAAGAACAATAAACAGGGAAGAAATAAATAGGTAACTTCAGATAGCGATAAGTGTTCTGAAGACAATGAAACTCTTAAAAACTTCATGATA
TGATAAAAGAGATTTGGAAGGGTAGCTGTAAATTGAATGTCAGGGAAGACTTTTCTGAGAACACATCTGGTCTGAGGTCTGAATCATCTATGTG
AGTCTAAACAAAAATAAATTTTTATCCTACCATACTTTCGGAACACCTCTGTGGATTTATGCCCCCTACACAGAGAGCAGTCTTCCAGCAGACA
CTAGCAGAATGTCTTGCACTTAATTTTGACTCTACCTGGTGTTAGCTTCAGACCCCAAGTTGTAGCTCAGTCCTACAGAAAGATTACCCCACCT
TCCCCACACTGTTTATAAATCAGTATTTCTGCAACCCCCTCCCTGAGTCCCATTAATTTTCTAGGACAGTTCGCAGAACTCAGGGAAATGTGTT
TACACATTTATTATAAAAGATATTACAAAGAATATAGATGGACAGTCAGATGAAGGAGATGGATGGGGTTTATGGGGGCTTCATTACATAGGCA
TGATTAATTACATCATTGGTCATTGGTGATCAACTCAACCTTCAGTCCCTGTCTCCTCCCTGGAGGTTGGGGGATGCGGCTGAGTGTCCCAATC
CTCTAATTAAGCTGTTTTAATTCTGGTGACCAGCTCCCTTCCTGGAGCTATTTAGGGGCCCCCAGCCACTAGTCATCTCATTGGCATTCAGAAA
ACACTCATAACTTCAGAGATTCCAGGGGTTTTAGAAGCTATGCTACAGGAACCAGGGGCAGAGATCAAATATATATTTTTTCATATCACAGTAT
CACAGTGAGGATCTACAGGAAGGGAGTTTGCGGAAGAGGGAACAGCAAATGCAAAGGCCTGGAAGTGGGAAGCAGAGCACCCAGGCTTTACACA
ACCTCTGACAGCTGATTTTGTTCTTGCCATTGTTCATTCATGTCATATCGTACCATATCATATCATATATATGGTTTATCAAGGCTATATTGAA
GTTATAGAACACCAGAGTTGGGAGAAACTTAAAGATTACCCTAAGTCCTGATTGTATAGGGGTGGCAAAAGAGGTCACAGAGCTAGAACCCAGT
ATCTTTTAGTGTGGAAGGAAGGGCTGCCCTCTGAGTCCTTGCTGTCAGGGCTTTTGTTACCCTGTGCAAAGTTAGACATGGCTTCAGGGGAAAA
TGGTCGAAAGTCTGATGGTGCCAGATACGGGATGCATCCCAGGTCAGCGCTTCATTCTGCGTGCCCAAGGTTGAGCTGACCATTACTTGCCTGG
TCCTTCACTTAGCCTCAAGTCCATGTTGACAATATGTAATTCCAGCCCACCTCCTCAGAGACAAGCAGTGTTTTTAATTGTTTTGTTTTCCCCC
TAATCTCAGAAGGTGGTCACCCAGGCAGGGACATGCTAGCTTTTTTTTTTAACCCAGTGTAATTTTGTATGTCTCTTAGTGCATGGAGACCTCA
CCTTGCTTCAGGTGGACCTGTCAAAGCTCAGCCCTCATGGTACGGTGTGATCATTGGACATGGCTGAGTCCATGTTTAAGGAAAGCCCATGCCT
TTTCTTTCTGCAAGTTGATCCTTTAGTACTCAGCCTTGCTCATTATATTCACCAAACATAGAATCCAGCTCAGTGACTTGAGGAAGTTGTTGCT
ATGCCTTTTGTGAAGTGAAAACATCGGTTCAGGAAAACAATTCAAATTTTGATGCTGACATTTGATCCTTTCCCCTACCACTTCCTGAATTCCC
TAAGCCAGAGCTCTTTGACCTCCAGATCCTACTGAAATACACAGTTATACATACTGTTTGTTGGACATGGGGGTTTTATTCAAAGCAAACTCAG
ATGCAGCATTTTTGGCTGGGCACTTCCATACATGATTTTAGCATGGAACCCTATCCAGGCTTCACCTGTGTGAGCTTTGTCTTGCTTACTGCTT
TCCCCCACCCCCCTGTCATGGATGTTTCTAGAGGACCTTTTGTGAATGTAACTTCCAATGGGAATGTTTCACAGTAGAAATATGGCTCTCCTGT
GAGGCTCGCATGTGACATTTCTGAAGATTTATTTAATTGGACTTTACTTAAATCCCATTGGGAAACATACTTTTGCTGTGAAGCTGTGATGTGT
TTAATTTGGGGTCTAAGATAATGTTGGTCCTCGGGGTGGATGGTTGAGCAATAGTTTTCCTTTTCTACAGTGGGTTTGTGTATGTGMGTAGCTT
TACTGGGCTTTCCTCTGTTTGTTTGTTTAGCAATGCTTCATTATTTAAACTTCTGCACTGAAAGGCAAGAATGTTTTTAGACATTCTTTAGAGA
CTTAATGCACAATATCTCCTTGAGCTCTCGGGAGAAATATGGCTTTGTGTTTTATAGCACTCTCCTCCTGTTPACATTCCTCTCTCTGCAGCTT
CCCAGGATGATCTCATGGATTTTTCCCCCTCCTTTAATCCGTCTTGATGGATTAACCAGTATTAAGTTTGAGACCAGTGAGCAAAAAAGGTGGA
GCCCTAACATGGTATAGAGTAGCCTCCCCAGTACGCAGAATGTCTCCAGCTAAAATATTCTTCCGCCCAGAAGGATGCCACATGGAGGTCATGT
GCCAGTGCTGATCTGAAAGGGCATATATCTAATGAGGAGGGAAGTGTCTTTGGGAGAGGAGTGGGCTTCTGTGCGTGCATGTAGCTGGTTCATG
GAACTAAGGTTCTACAAGGCTTACTGCGCTTGCCTTTTATAGGCATGCTGGTCCTTTCAGCACACCCCCACCCCCTGCTGCCCCCGCCCCCACC
CCCACCACCCTGCCACCCCCATTCTGACCTTGGACCATTTTTGGCTTGGCTTTAAGGTATGACACCAAGTTGGGTTGGCTCTCAGGGGCTTTGT
TCTCAGGAAGCCCACAAGTTTTAGTGTGGCTCTTCAAAAGTAAGTATTATACGTTGTATTTCTAAAAGTAAGCTACACTGGGAATTACAAGTGG
CACTATATTCATTTGGGCTGCTTTAACCAAATGCCGTAGACTGGATAGCTTAAACAACAGAAAGTTATTTTCTTATAGTTCTGGAGGCTGTAAG
TTCAAGATTGGGGTGCCAGCATGGTTGGGTTCTGGTGAAGCCTTTCTTCTTGGCTTGCAGACAGCTGTCTTCTTGCTGTGCCCTCACAAGGCAG
AGAAAAACAGACAGCAACCTCTGTGTTCTCTCTTCTTATAAAGGTACTACTCCCATCACAGGAGCCCTACCCTCATGACCTCACGCTGGGGATT
AGGGCTTCAGCATACGAATTTGTGTGTGTGTTTGAATATGGGGAGGAGAGATACAATCAGTCCACAGCAGCCACAAAAAATGCATTCAGAAAAG
GAAAGGGTATGAATTTTGCCCAGCATTTGGAGTGTATGTAAATTACACCATTTGGTAGATCAGTGCGTTCTCTCTCTCTCTCTCTCTCTGTTTC
TTTTCCCCTCCCCCCTCTTTTCTTTTCAAACACATATTGATCACCTGTTGTGGACTTTGGAACACTCAGGAGCTTCCCACTGAACTCAGAATAA
AACCTGCCTAAGAGGCCCTGTGAGGAGQGTCCCTGCCTCCCTTCCTTCCCCTCTAGCCCTTCTCCTAACCCGCCTCTTGCCCAGTCATTGTGCC
TCCCCACATTTGGCTCCCTTGAGTCACTCAGAGGTTCCCTGAGTTCCCTCCCTTTCCCACTGTGGTGTTCCCTCCCTATGTTCTCCTGGATTCC
GTCCCCTCTCATACCTCAGAGAGGCTGTCCATGAACCCCAATCTAATTAGTCCCCTCTCATAGCACCCAGGGACTGGACAGCGTCTTCCGTTAT
GACTTATCTTAATTTGTGACTCCCTTCTGGAATCTGTGTTTATCGTTGTCATATGTTTCCTCCTGATGGAGTGGGGACGGGCAGGGAGGGGGCC
TCTGCAGCGTGTCACTCACCTCCTCAACTCCCAGAATGTTTGCGGAGGGTTTGATCTTAAATACAAGAGATTATAACAGTGAAAAATGTGTTAA
TGTAACTGGCATGTCTGTTAACAAATCCCAGACTTGATCTGAGTGTTTATGGTGTGCATTTCTCCCTGGGAACCAGTTGGTCTGTGCTAACATA
GGTAAAGTTTAGACACGTACCAGAAAGAAAACTGAACTTGCAAGTTTTCAGGACATCCGGGCTGGCTACTTGGCTGTCTTTCTGTCGGCCCCTG
ACCCCTCACCCCCACACTGGATCTTGGTAGGCACCCTTCTTAGCCATTCACTTGATTGAAGCCCAAAATCCTTTCTTGGAAAGCAAAAATACTT
ACTCTTATTTCTTTAGTTGGCATTTTTGTTAGTGAGAATGGAGTTCAATAGAAGTAGACAAAATTCTGGTGGAAAAAAAAAATGAGACTACACA
GGCTGGGAGGAAGGTCTGTGCATGGAGGGGACAACTGGAACCACCAGAACATCTGTTCTGACCCTTCTATTCCACACTAGGATTAAACCGAGAG
CAGAAGGCACTTCCCTGCCCCTCTGGCAGAGGGACTGACTTGAAGTCAACTATGCCCACAACCTGTCTTCAGGTGGTTCTTACTGAAACCTTTG
TGAGACAGAATGGGGAGGGTGAGTTAAAGATGGTATTCTCTGCACTAGAAGAGCCTTACTGTGTGTTTGGCTGGCAGGGATGAGTGCTCAATCA
CTCTTCCCAGCCCACCTTTGACTTTTTAAAAATAGGTCTAACAAATATTCTGTGGATTCGTTTGAAATTCAAGCATCAAAATGTATTCCAGGTA
GAAGTCTCTCTTTCATGTTTTCTTCCCATCTACTGCTGATGGATTTATGAGAGACAGAGPAGGTGTAGGGAGAGGGAGAGAAGGCTAGAAGAGG
GGAGATGGCAGGGACAGGGAGAGAAGAGGGAGAGGGGAGGGGAGGGGAGGGAGGAGGGATCAGGGTACAGGAGCAAGAGTTGGGGAGGAGACGT
GAGGGAGAGAAGAGTGGAGAAGGGGGCAAAGGGGATAGAGATAGAGACCAAACCCAAGGCCCTGGTGGGGAAGAAACCAGAGAGAGTCATAGAG
TCTCACTGACTTCTGCAAAACCTGAGGGGCTTCCTGGCTCTGGGAACTCTTGAGATCTTAACAGTTCAAATCCTGCTCACATAAAAGTCATCTG
CTCATTAAAATAACACAAAGCTCTTTTTAATTGTTGTCTTTGTGGATTACATGACTGTCATCAGCTGTTGCTTCTGGCAGTGCTTTCATGTTCT
AATGTAAATAAATGAGCTAACACATTTAAAGAAACAGGCTGTATTTACATACTTCTGAGGTATCCTCTCAAAACAATCTGCCTGTGTTATTCAA
ATACACACAAATTAAACTTGTGGTAGCTCAAACATTAGTTATTCATTAAAGCCTTAAAGTTGAAATGGTAGGAGGGGGGCAAGAGAACTGCACC
TCCCTTCAGGGGGTAGGAACCAGAGGCTGACTTTTCTGTGTGGACTGAGCTACTCCAGCCGGCGGAAATCCAATCCCCAGCTCTTTTCTATACC
AATTCTGCCCTGAGGCAACTTCTGAGCCAGGAGGGGAGGGTGGTGAGAGCAGAGGGGAATCTTTAAAGCTCTGACAAAAACTTTCCAGGGTCAG
CATTTCTTGGAAGGCCTCCAATTAAACTTTATTTTCAATTTTCATAAATCACAATTCCTAAATCATTGGATGTTTCTGACTCAGTTTACTTTTT
TGAGGTAAATATGTGGGCAATAATTTTTTTAAAATTCTTCCTATTGAGGGCATGGCAAAGGTTTTATCAAACAAGGAGGAAACAGTATATTCGA
AAATATAAGAAAAATGACAAGAGACCCCTCCAATAAACAAGTTAATGACTGGAAAATGTAGAAGTATATGGAACAAAAAATGGAGAAGAAAGCA
CACTGCTAATTATACAAAGATTAGGAAGTAGCCAAAGTGACTGTTTTAGTTCTTGGTAAGTGTCATTGTCAAGTATTAGCTTCTTTCTGGAATG
CTGGGTTTTTTTTAACGTTGGGGTGGGAGGGAATGGCACATCATCTGAGAGAAACAATAGTAACTATTGAAGTGGCTACCTTGTCTGGGTTAAA
TAACCGGGGTTCGTTGTCTCACACCAAGAAAATTTAGGACATAGACACACAAGAGGAGTTTAGAAGCAGAGGTTTAATAGGCAAAAGAAAGGGA
AAGGAAAACAGCTCGCTCTTTAGTGAGAGAGAGGTGACTTCCCAGAGGAAAAAGGCCGACTGGTGGTGGATGTGCCAGATTTTATAGTCTGGCT
TGAGGAGGCAGTATCTGATTTACATAGGGCTCACAGATTGGTTTGATCAGGTGTGAGGTTTACACAGCACTCAGGGGAGGCTGGTCACCCCACC
CTAATCTTATTATGCAAATGAACTCCCCTTGCCTGGCGCCATCTTGTCTGCTCCTTACTGTACACGTGGCCGGCAGAGAAAGGGGAAGATGGAG
ACTTTACAGGCTGCTCTCTGTTGAAAATGATTTGGGGCTGCTTTTCATTAAGGGGAGACGTTACGGAGCACTTCTGTACCCTGAACTATCTGCC
TAAGTAATTTCTTCTTAACTCCTGTATCACTATGATACCAAAGTTACTATACGATGACAACTCAAGCAATGTCTATTTCATGTAAGCCTCAGTA
TTCTATATTATGTTGTTTTGTCCAGACAATCCAAAGAAAAGACTATTGTTAACTCAAAATTCAGGCAAATTCTGGTATCAACTTTTCAGAATTT
TTTTTTCTCTTAAAACATTACTAAATACTACTTGGTTAGATGAACTAAATAAAAATGAACAACTTTTATTATTGGAAGATAGTTTTGGGAGGGC
TATTCTGGAAATGATTTCAGCCTATCATTTATCACTAGGAAAACACCAGGAAGATCTTTTTATTTTATTAAGTTTTTGTAAATTATTTCCAAAA
AGAATTGACCACCTATCTCAATACCTCTTTCCCTGTAGCTCCTATTGGTATCTAGCCATATCAGCACTCACCTGTCTTCATGTGACTTGGGGCT
AATTCCTGTGTACAAAGGGATTTAAATAAGACTAGTCCTGCTCTCCAAGGACCTAAATTCTATTAAAGCTCTTAATCTGGATATACACAGAAAG
GAAATACATTTTAAAAGAATATGTATTAATAATGCAGTTATAGTTGTAGAATGAGGCTGCATGTATACATATAATAAGTTAGTTGAAACTAAGT
TTTTATAGAATGACTCTGAGAAAAACTGTAAACACCTCAACTTATTGAGGAAATTGAGGTATGAAAAAGTTGTGTGTGTGATAACTTTTCTGTA
CGTCCATTTCAAAAATTTCTAGAAGTATGCATATATTTTTAGAGAATATACCAGAAATGTATTTTTGGCTATTCGATATGCCTAAAAAATGTCT
TTTTTTTTCAAGGCTTCTGAAAATCGTAGACATAAAGAACTAAATATAATGAGGTTTTCAAGGAGACCACACTAACCACCAGCCCCAACCCCCA
CTCTGCATGCCACTTCCTCCGGCAGTGATCCAGTGTAGACTAGGCTAACACCACTGAGTACACTTTTAGCAGACCAGAGCTACAGTGGGCTGTC
CTCATGGCTCCTGCTTTGTCCTCCCTCTCTGGTGATGCCCTTGCATGGGGTTGCCTCTGCAGGATGGTCTCCTTTTCCTACTGCTCTGGTGCAA
CTGTACCAGCATCAAAAGTTCATCTCTAGCAACACTGACATTGACCCCTGTAGTCTCTGGTCATCTTCCTGCATCTCACTACCTCCAGTGCTAC
TTAGCTTTGCTGCTCCATTTGACAGTCATTCAAATAGCTGCAGGGTTTGAATGCCTTCTTAAAATACCAGGATGGCTATTGTACACACAAAATC
ATTGAATCATTACGACAGACCTAAGTAGTAGTATCCTATTTTATAGTTATGGAAAGATAGCCCAGAATACTTCAATAACTTTCAAAAAGTCACA
CACTTGGCATGTGGCAGACCCACTATTCTAAATCCAAGGCAAAGCTCTTTTTTTTTTTTTTTTTTTTTTTGCTGCTTTATATATCATCCACTGT
CTTTTCTAGAGTTGGCTTTTAAGATATATCTGTAGATAACTGTGTCTCCTTCACACAGGAAAAACCCATGAATGGAGGGACTAACTTATTTTTA
GATATATCTTCCTAAAAGACTTAATAATATAGCAAGCCAAATAATGTGGATGGGTACTCAATGGGATTCAAGATCCCATTATGTTATATGAGTA
TATCTGAAGTTTTATGTATATGTGTGTATTTTAAGCTTGTGATACTTCCTTGTTTATGTCTTTTCAAACATAAGGCATCTCACATGTAAGCCAG
ACCACACTGGTTTGAGGGAAGCAGTCAAATGACGTGATCATAAGAGACTTCTCTCTGACTCATAGTCAGCCTCCTAGCTTCTCTTTCTTCCCCT
GAGTAAGATAAATACTTCTTTATCTCTTGTTTCATACCTTATACCTAGTGAGACAGCACTCCCATTCAACTCTCTCTGCCTCTGTTTGATAGGG
CCTCCCTTTACTCTTCTTGGGAAAGAATACAAGTTCCC1GGGGGCAAGATCCTGCGGACAATTTAGTACTTACTTCCTTGGGTATTTTACAGTA
GGAATGATTTTCATCTTGAACTTGAAAATACCCTACTGTAAAATACCCAATTCCTACTGTAAAATACCCAATCACATCTATGGAGTATAAAGAA
CATTTCATAGAAAACAACAAGTAGAAATAATCTTTGTGGATCCAGCTGGTACCCACCACCACCCAACATGTTTCTCCTGTTAAGATAGGTGATA
TTCTGCCCTGAACTTATGAAGTGACTCTGACCCTGTCTTTCAGAGAAGATTTCAGCCTTGGCAGCCTGACATCAGCTCGGGTTCATCGAGGCTT
TGTTCTTCCACAGATTCTTGTGCTTATAGAATCTTCAGTGGCTCTGATGTTCTGTTTCCATCGAGTATATCATAGCATCCATCTGTTCCTGGTA
TAAACAAAATAGTATCCAACTTACAGCAATGTCTCTCCCACTTATAATAAAACACCTAACTCTCATGTAGCATTTTATCCATATTGAGAGCTCT
GTGAGATAGAAATTATTATATTACAAGTCACAGATATGTAAACTGAGATGCAGAGACATCAATAACTTGCTCAAGGTTACAAGGTAAGTGATAG
GAGTCAGGAGTTTTAACTCAAGCAATCTGGCTCCAGTCTATCATCTTAACCACTATCATCAACCTACTGTTTTACTTTGACGGACTGGTTACTC
AGTAAGAAAAACTAGGCAACAGAGAGGGGTGATCAATATGCTTATAGCAAACTGTAGAAGAGTCCAATTTTAGCCTACACCCCTCCCCCTGGTC
TTTGGACATTACTTCTGTTTTGCTGACCTTCAGTGAGAGTGTTCCTTAAATTTCTGGAAACAGCTCACTTTCTAGCTTAGCATAAGACCAAAAG
GGCTGTCCACCTTTACAGTATAATCTCTTGCTTATAGATTCTGCATTTGGATGTGGTTTCTTTAACTCACAACCCAGTCGCGCATTGCTTCTGA
ATCACATTACAGAGGACTCTTCTGTTTCTAGGACTGCAGTCGCCAAAACAGCAACAGGAGCTGAAATGCATCTATAAAGAGAAGTTTCCCACTA
AAACTAGAATCCATTGCTTCAGTAGGACTGTCACTGAACTGACCGTGTGTTAGAAGAAACTGGCAGGCAGTATTTCCACATATAGTTCTTTAGG
GGTGCTAGGTATTCTTTGTTGCTGCCATTTAGAATCAATGTACTGACCATACTCATTTTGGCAGAGAACCTACTTTTGACATAAGCTTTGAAAA
TGTTTGCTGCCCCCCCACCCCGCCTTGCCTCACCCTACCCCAGCATTGTTGCTCATGGCTACAATATGTTCGCACTAATAACTGCAGGCTCTTG
TGCACATAAGGAAGTTAGCCACCTTGGTAGAAATTAGAGGAAAGGAAGAAGGAGGGAAGAACAGTTTTATTTTCAAAATCCAATTCTGTGGAAA
AACCAGAAATGCAAAGACAGTGTTTGTTAGGAAAGCTAGGACCAACAAAGTGAATCAGATATATTTGTTTAGGGGCAAATAGGGAGCTGTTGCT
TTCTTTATGTTAAAAGTATGTTTTGCAAACTTTAAAACTGCTCGGATACCTTGGTTCCTAATTCTTTTTTGTTAAATAAAATTTTAAAAGTAAA
AACTCAGGAACATTAGTTTACTTATATAGAGTCTTTATATTAAGCAGATTTTTTTTTTTTTAGATCTTTAAAATGGTAGTTTCCCAAAGTGATG
AAAAGAAAAAGCACTTACAATGTGCTGGACATTGTTTTAAGGTACTTTATTAACCACTTTAATCATTACAACAGTACAGTGAGGTAGGTACTAT
TGTTATCCCTGTTTTGTATACAGCGAAACTGAGGAACAGACAGGTGAGAAACCTGCCCAGGGTCTCATAGCTAGTGTTTGCCTCTGCTGGGGAT
TCAAACCCAAGAAGTCCAGCCTCAATACTCCTTCTCTGAATCACTGCACTGCACTTAGGGCTCTCATGGACTATGCGTGTGCATAGATCTGTTG
GAATTTAGAACAAAGGTGATGATATTCGGGGTAATAAGACATTATTTATCCAATACCTGGTCGTGTATCAAACCTTTACATAGGCTTCTCTGAA
CTATGACCCTGATGGGTACATGATATTACATCCACTTAATAGATTGGTAAACTGACGCTTAATGAGGGaCCTGACTTGAAACAGGCTCTCACAG
CTGCTAGGAGGCAGAATCATAACTGCAGCTAGGTGTGGTTTGTCTCTGACCTTTGCTCTTAACCTCCATGCTACACTGCTCCTGCTTTGTTTGT
TAATTATCATGTTCTAATATTCTTAAATAGTAATAAACATAACATCATCAGGGTTCCTCATTAAGTGATCAGTCACTTTGGCCTCTGGTGATAC
TTGATGGGGTAGGGTCGGAGGAAAGTAACTGGAAAAGAAAAACCAGATTAGCTTTACTCTTCCCCCTCCCCTCCAGATTTCAATTTCAACCCTT
CCCCAAGAGAAGAAAACAATCCTTCCTGCAGACTTCTCTGGAAAAAGCCACCACTGTGAGCTTCCACTCGTTGTTTTTTACTTTAAGCACCGTC
CACAGAGAGAGTTTTTAACTGCAGTACAGAGCTGTGCATAAGCCTTATGGAAAACTGATGCCCAAGTTGATTAGCAGAGTGGCAGTTGAACAGG
ATTCTGTGTGTCTGTCTCAAATTCCCTCCTCCCCCCACTCCGCCACGGGTTTTTTTCCCCCCATTAGGCAAATGGAAGGCCCATGCCACCTGTT
CACCTCCAAATATTATAAGAACAGAGTTCCTACATGGCTTAAACATACAATGCACCCATACTTTTGCACATAAGCATGCTCTGTGATTTGGAGC
ATTCTGTGTGTCTGTCTCAAATTCCCTCCTCCCCCCACTCCGCCACGGGTTTTTTTCCCCCCATTAGGCAAATGGAAGGCCCATGCCACCTGTT
AGCATTACATCATTGGGTCCCCAGAACACAGTTGCACCAAAGGCCTTAAACAAAGTAGTTCTTCTTGTATTGTTTGCACTCAAAGAGCTGATAT
CATCCCAACTGATCTCATTGTATGTCTTTGTGTAACTGCTATGGCAACTGGGCAGGTGGGGAGCCTCCAGCTGATGTCATTGAGAGAGGAGGTG
TAGAGGCAGAATTTPAAAAAGCTGGACGCTGCTTTTTTCTGTGTGTGCGGTTTTCTAGCATGTGTTGGTTTTTTTTTTTTTTTTTAAACTAGCT
GCTTTTAAGACAAATTACAGCTTGAGTACCCTAAATAAGAACAGAGAGGTTTTTCCCCTGCTTTTAGAGCCTTCTTTTGAATTTGTTTTATTGT
TCTTCCCTCTCATCTTATATGGAAAAATGTAACAACTGCCTTTTTTGAAGAAAAAAGTGAATTAGGTTCCCATCACAGAATCTGTGTGGAATTT
GGGTTTTTGTGGAGGCGAAGCTGAGCAGATTTTAACTCTTCAGAATCCCTTTTGTGGAGGCTGTGATGATGACAATGGGTAGTTTTTATCTTAT
TTATTTTTTAAGTCTTCACTGGTGAACTTATTTTCCATATTTTCTTTCTTCTACTTCAGAATTATCTCTTCAGGTTCAAACTTTATTGTCATAA
TTGCCTAGCAGAATAAACTATCATATGTAAGGGCAGAGGTTAATTCTAGAAATCAACTGAAATTCGTATATAGCTCTTGTTTTTCTTTTCAAAA
AGCTAGGGTAGGTTTTGACTTCTTACTTTCAATGAGTAAAGTGAACATAATATTGCCCTTTGAGTTTTCTTTGGATCATAACCATACCTTAAGG
AAAATATGATTTCTCTCTTTTACACAGCCAGCTCCGGGTCCATTGGTTGTGAAAAGTCATGAGACTCGGTCTAGGTTATGGTCTGGAGTTTCCC
GCTGTGGTTTGTCAAAGATGAGACTTGAAATGGAGAAGAACATATGCCAGGCTCAGAGGCTAGTGGACAGATAGCTTCCTGTCCGTTATTGCAA
CATAACCGTCTCTTGTTCATGCTTTSGGTTTTCTGCCTTATCTCCTGGAACTATCAGACAGCCACAGGGCCATCTGTCTTTGAGGGTAGATGTT
GTGTTCTGGTGGCTTTCAGCTAATAGACTTTTCCACTTCCCCCTCCTTTTCAAAGGGAAAGCCAGAGTGTATCTAGTTCTGTTTGAGTAGGGTT
AGAGCTCAGTTGGCACTAATAATGTCAATCCAGATTTTATAGCACTTACATGTATATTATTATAATTTGAGCTTTGTAATAATCCTTTGGGTTT
ATCTGTGCATGCCATTGTGTCTAAAAATATTAGCAGAAAATTTAAACAATAAACTATGATCCGTAAATTCCCTAATTTAAACAATTGTTCGTTT
TTTGTACAGGTTCTTCAGCTTTTCGTCAATGTTTTTTACTTAGCTTTTAAAAATTTAATATTAAACCAAAATGTTTTATTTATTTTCACATCCT
TTTGCAACTACTCTTTTAAAGGCTACTATAATACTCCGTGGAATGAATGTAATAACTTTTTTCTTTTTATTTTTGTGTTTTACTTGGCCTTTTT
AAAATATATATAAAACAGATAACATTGTAGTGAACATCTCCATATATCTAGCTAACTATCTTTGGTTAGATGATTTCCCTAAGATCCATTCCCA
AAAGTGAGATTGCACTTGTTCAGGGGAAGGTTTGCATTTTAAAAATGGAGAAATAAGCACAGTTTATAAGCTTTTTATACTAGACCCATAACTC
GTGGGAGGCAGAGCAGAGCCGTAAGCTCAAAAGCATGCATTCACACATCCAAAAGAGGTTTCTTGATGGGTTTATGTGCCCACAAGCCTTGGAA
AGTCGAGGAGTAAGCAACCGCGCTGCTCTCACATTCTGTTCTACTGAAAGTCAGGGTTGATAAATGTCAGGGCCCTACGTGGAGAATATCAAAA
AAGATTATAAGAGCAGTCCTTCACACCCCACCCTGGCTTGTCTTTTCCAAGTACTTCTCTGCTGATTTCACCTGTGGGCAATCATATAAATAAT
ATAAATTAAAATAGTGAATGCCTGCTGAGTTCTACTGTGTGCCAGGAGATGTGCTAAGTATTTTACACGTATTCATTTATCTCAGGCTCACAAT
TGCACTCAAAGGGAGACACCATCACCCTCAGTTTACAGATGGTGGAACTGAAGCACAGAGATAAATGAACTTAAGTAATAGTGTCAGGAAATGA
TGGTACCCAGATTATAATCAAGATAACCTGTCTCTTCCAATGTTCCTGCTTGTAACCACTCTGCTGTATTGGGAGCAAACTTGTCCCTCTGGGT
GAGGTGAGCTATGAAGGGTGCAAGACATGCTCCTGTGGTGCTGAGATTCCACCCGAGGAGGTCATCTGTGCCTTTCTTGGCTCCCAAAGCACAG
GGTTCTATGTAGGCCACTGACCATATGGCTCTTTGAGGCAGGTCACATTTGCAGCTGACCAAGATTGCCTGATGCCGCGTGGTACTCTTGCTAC
GGGAACATGCCCTCATTTGTAGAAAGAGCACATTTCCAAAGTGTTCCATGCAGTGATACTCCCGAAGCTTGTCTTGGGTCTCCAAATGTGACTT
TTAGTTTTTGTTTTGTTTTGTTTTATTTTTATTTTTATTTTTTTTGCTTAAATACATATTTATATATGTATTTAAAATATGCATGAGCTACAAT
GCAAACTCATTCTAATGGGCAGGTGGCATTCTCTGCCAGCTCTGATCTCAGTCACACTAGCCCTTCTGTGTTTGTGTTCTAGGTAAGACTTACT
AGTTTTAAGCAGAACCATGAAAACCTCTGTGACAACTCCCTCCAGCTCCAAGAGTGCCGGGAGGTGGGGGGCGGCGCATCCGCGGCCTCGAGCT
TGCTACCTCAGCCCATCCCCACCACCCCTGACATCGAGAGACGCTGAGCTCACCCCCATCTTGCCCTTCCTGTTCCTTCGCGTGAGCAGCATGC
TCAGGACCTGGACACCATGCAGCGGCTGAACATCGGCTACGTCATCAACGTCACCACTCATCTTCCCCTCTACCACTATGAGAAAGGCCTGTTC
AACTACAAGCGGCTGCCAGCCACTGACAGCAACAAGCAGAACCTGCGGCAGTACTTTGAAGAGGCTTTTGAGTTCATTGGTAACTATCTCCCAT
AGGGAATTTTCATCCGCTGCCTTCCTTTTCCCTCCAGCTCTTGCTTTGATATCAAGTTCAAGGTTTATTCCTGTGTTGGAAAAGAAAAGCTGCC
CTGGAGTTTTGGAGGGCTCTGCCACCTGAAACAGCCTAAGCATTCTCCTAAGTCATAGCCTGTCGAGTGTATCCAAGCCATCAGGATGGATATA
AAAGGACAGTAATTGAATTTGTTTGGGATCTGTTCAACTAGAAATCCTCATCCCTCAAGTTCTCAGTGAAATAGTGGTAGGGTTAGCATTTATG
TGCTCTGTAACCAGAAAAGTGGATGGAAAGTAAGGAATCGGAGGTCAGAGCTGGTGCTACTGCTACTTGATCTACTAGCGCCGTTGAGCACCTT
GGAAGCAATATCTAGTGAATATGACACCCCTTCCAAACATACCTATGCCTTCCGGTCACCCTACTAAGCCTTATTCATCCAGCATAAGGGGAGA
TGGTACTAGGTTTCCTTTGACAGTCTGCCCCAAACTCATTGAACTAGGAAAGATCAGGTTGTGTAGACGGAGTAGTTCCCAAGGAGGAAGTTTT
AGCAACAGCACGCTTTGATCCCCAGAAGGAAGCAACTATTAGCCTAATCCTAATCTTAATTTCTGCAACTTCTATAGCCCCAAACTGCTATAAG
ACTCAAAATATCGCCGGGCGCGGTGGCTCATGCCTCTAATCCCACCACATTGGGAGGCCGAGGCGGGTGGATCACCTGAGGTCAGGAGTTTGAG
ACCAGCCTGACCAACATGGAGAAACTCCATCTCTACTAAAAAAATACAAAATTAGCCAGGCGTGGTGGTGCATGCCTGTAATCCCAGCTACTCA
GGAGGCTGAGGCAGGAGAATCGCTTGAGCCTGGGAGCGGAGGTTGCAGTGAGCTGAGATCACGCCACTGGTACTCCAGCCTGCGCAACAACAGT
GAAACTCTGTCTCAAAAAAAAAAAAAAACACTCAAAATATCATTTTGGGAGCACCTGCAATATAGCAAAACTCAACATTTATTTTTTTTTACGT
AACTTCAACTTTTATTACCATAAAGGGGTGGCCTAGTACTTTTCCTAATATTACAGCAGGTTGTTATTCCACGGACTACAGTTTGAAAGTGGAG
TGGGGTGTTCCTTTGTCAGAGAAGAAAGAACAGCCTCAGGTTTACAGCCAGTGGGGTCTGGTGGATTTGCTGTAGTGTTGACATCTCTAAAGGT
CATTTTGCCAAGCTGTTTTCCTTGCTAATTATGCCGTCAGCACAGGACCATGGAATATTGAGTCTGAAAGGAAATTCAGGGTGATCACTGAACT
TAGAGAAGTAAAGTGACTTCTTCAAGGTCATAGGAATTCTAGTGTCCAAGAAGGGGCCAGAATCCAGGGTTTTTGACTCCCAAGCCTGAGGCCT
TTTCACAACACACTAAGGCAATGTTTATGATGCCCTATTAAGTAATTCCCAACTGGCAACACTGAGGATGCAGAGATACTGCAGATTCTTTATT
AATAAAATCTGAGCCAAACCTGGAGCGCTGAGGAAGAAAAATTTCTCAACAAGCATATGAGGAACTTCTGGTTGATACATGATGTGATGTTATG
AGTCTTGCTTTAGTAAAAACACAGCAGGTTTTATAAACATAATAGCTTTCACTGAGAGTGTAATATAAGCTGAGCAGCATCTAAGTTTAGTGTT
TATACCAGTTTATTTATTCCTGAATCAAGTGTGTGTAGTGGAACCGGTGGTCCAGCCAGGCACTCAGAAGCTCATTCTCTCACCACCATGCTTT
ATAGCATTCAGCCCGAAGAACAACTCCATATCACAACCTGCATAGGCACAATTTTATTTTCATTGTTATATTATTGGATAAACTGAAATCTTAC
AGTCTTGCTTTAGTAAAAACACAGCAGGTTTTATAAACATAATAGCTTTCACTGAGAGTGTAATATAAGCTGAGCAGCATCTAAGTTTAGTGTT
TATACCAGTTTATTTATTCCTGAATCAAGTGTGTGTAGTGGAACCGGTGGTCCAGCCAGGCACTCAGAAGCTCATTCTCTCACCACCATGCTTT
CTGGTCTTGACTTGGAGCTAAATACCGTGTGCTGAAGGGAAAAAGTTGAAAAAGAGAGATGCTTACTTTTCTTGAAATACTGTTGACTATGAGA
AGTAAACTTCATCTGTAAATTTTTAAGATGAAAATTTACATCAGGTTTTTTTTCCTTGGTCCACTGGTGGGTAGCCAGTAGATGTAGTTTAGGT
GAAATCACTACTCTCTCTACCCCTTCTCCTTCCCTACCTGTTTATCTAATTGGTGTTCCTTCAAGGAAGCTTTCTTAGGGATGTGTTAGAGTCT
AGAAATGTCATTAAGAATCTGTTCAGGTAGTGCCCACATGCTATTGTCCTGAGCTTTTAATATATTTGAAAACTTAGCAAGTCTTCCTCTTTCA
ATTGCTCTGGATTTTGAGCCATTGCTGGGATTCCTCCATCTTCTGCATTATTGCCACACTGGAATGTCATACATGGAAGCAAACGTGATTGAAT
GCTTGAGAGGTGACACCTCGGCCTGTATACTACTAGCCTTTAGTTAACTTTGCAACAACTGGATTTTCTGGGGAGAAGTCTGAACATAGTATCT
TCCTAGTTAAGCTTTATTCCTTTAACCTTAATCAGGGTTTAGTAAGACTAAAACTCTTATCCATTTAATGCCCGTTCCTGTACAGAGCAATTCT
ATAGAATAAAGAAAGTGTTTTTTCTCTATTACAAGTGAGAGAGTTGTTACTAGCACTAGGTTTGTTAAGTCACACAACCGAAAATTGGAATTAA
CATCCTTAATCCAAAGAGGAACTGCATATATATATATATACACACACACACACACACATACACACACATAGATATACATATATATGTGTGAGTG
TATATTTTCATGTGTGTGTATAATTTTTCCCCTTCCTCTCTCCTTTTCCTTCATTCTTTTTTTTTCTTTCAGTAGTGTAATTCCAGAAAAAAAG
AGAACAAGACATTTGAGAGAAGCTTCAGTGACTGTTAGAGTCGACTTGATCAGTAGCTTGTTCAACGTGAAAGTTAACTGTGTTATAGGTCTGG
CTTCCGGCCACTGCTGTCACCTGTGCAGCAAACTGTTCCTATCAGCAATTCTTTCATTTAATCTTCTCTTTAAATGTAGATGTATAGCTTCTTG
TTCGTGCTGATATCAACTTTAGCCAGTTCTGGAAGTTGTGACATAAGTATACGTAATGAACTAGTGGATTATACTTGGCTTAAAAACTGTTTTA
ATTCATTATTTCTATACAAAATAAGGCATTGCAAAGTTGAGATGAAAGTTTCTCTCTCCGTGCCTCTCTCTTCCATCTTCCCTTCATCTCTTGT
CTTTCTCCCTTTTTTCAGAGGAAGCTCACCAGTGTGGGAAGGGGCTTCTCATCCACTGCCAGGCTGGGGTGTCCCGCTCCGCCACAATCGTCAT
CGCTTACTTGATGAAGCACACTCGGATGACCATGACTGATGCTTATAAATTTGTCAAAGGCAAACGACCAATTATCTCCCCAAACCTTAACTTC
ATGGGGCAGTTGCTAGAGTTCGAGGAAGACCTAAACAACGGTGTGACACCGAGAATCCTTACACCAAAGCTGATGGGCGTGGAGACGGTTGTGT
GACAATGGTCTGGATGGAAAGGATTGCTGCTCTCCATTAGGAGACAATCAGGAAGGAGGATGGATTCTGGTTTTTTTTCTTTCTTTTTTTTTTT
GTAGTTGGGAGTAAGTTTGTGAATGGAAACAAACTTGTTTAAACACTTTATTTTTAACAAGTGTAAGAAGACTATAACTTTTGATGCCATTGAG
ATTCACCTCCCACAAACTGACAAATTAAGGAGGTTAAAGAAGTAATTTTTTTAAGCCAACAATAAAAATATAATACAACTTGTTTCTCCCCCTT
TTCCTTTTAAGCTATTTGTAGAGTTTATGACTAAATAGTCTGTGCAGGTTCATAGACCGAAGATACTACACACTTTAAACCAATTAAAAAGAAC
CAAAAGTAAATAGAAAAGACATTGAATCACCAAGGCCTGGGATCAACCTGGGCTGTCCACACAGAAAACAAAAAACCCAACCAAACCAAGCCTG
TTGTGCTCACTGGTGCAAAGAGAAGATCAGGGCAGCTTAAGTGGTCTAAGAATCCTTCAGGCATTCTTTAAGGAGAAAAAGGATACCTTTGATT
TTGTGTGTTTCATGCTCTGGATTTTTTTTTTTTTTCCTTCTCTGGCTTTAAGACATTTTTTTTGAAATACTGAGGAACTGACCATTATATGCCT
TCACTGGCTTCTTGTGCAATAATATGATGTTTTAAGTGTGCAAACAAGTTAGAGCTGGCAGCTGAATGATAGACAAATAGTGCAAATTTGCCAG
CTTGGAGATAGAAACGAATTCAACAATATATCAAATACTTTCCTTCCCACCTTTTTCCTTTTTTTTTTTTTTTTCTGATTTCATTCTGGTTACA
GTGCCATAAACCTTGTTACATATGTATATCAGAATGTAAGAAAAAAAAATTTATTTAAAAATATTTTTCGCAAAAAAAAAAAAAATCTTGGTGT
GTTCAAGCCTTTCTTAAGCTCTGGTAACTTGCATATACAACAAAGTTGAAATAAAACAGATTTTTATATAGGGAAAGAGAGATGAGGAAGGGAA
GTTCAAGCCTTTCTTAAGCTCTGGTAACTTGCATATACAACAAAGTTGAAATAAAACAGATTTTTATATAGGGAAAGAGAGATGAGGAAGGGAA
CATATCCAGTTTTCTCACTGTGAACAGAGTTAGCAGGAAGTGGAAGAGCTCCAGTATTGATCTGCCAAGTCACTGTCCCTTATATATTCTCACA
ATGCTGTCATCCTCTCCTGCAATATCATTCCTTTTGCATCGGGCAGGCTTGCATTAGAAGGGGATCCCATCTGCCTTTGCGCCTTGGTGACCAA
ATCCCTGCCAGATTGATTGGTGAGAATTCAGTGATCTCCCTAGTGGCTATTTGATAAATACTTAGGACAATCTTCCATTTAGGGATCTTGATGT
TCCTCCCCCCACCCCCCCACCCCATTGTCACTTGCTCACTTGGCATCTTTGGTGACATTAACCATGTAATGCGTTGCAGCGAATTGCTATTGCC
TACTCTTTTCCCCAGCAAACTGAAACTCAGCTCCATACATGTGTACAGCTTCCTTATAACACTGGGTTAATGCATAGCTGGGTTCCCAATTCAC
TGTTGATGGAGGGTTGGGGGTATGGGAACACAATGCAATATTTTCTGTGTTTCATTAAATTAAAAGTCTAGTGAATCGGACTTGGCCATAAGGA
AGATTTATGGCAAACTGAAATTTGCCTGGGGTGAATAAAAGAGTTGAAGAATCTGGCATGTTGGATTCTATGTGACAACATTTGACATAAATAA
TTTTGTGTATGTTGGCTTGAAAATGTTAGGAAATATTTTTGACATTTAAAAAAATAGCTTTTCTACCAAAAATAATTTAAATTTTTCTTTTATA
GAAAAGTGGGAGACTGAATTTAACAGGAATTAACAGCTGTTTTAGATTATTTTAATAGAAAGTGTTAATCAGGTGAAAAGCAAACTCTGCTTTG
TGCTTTCTACTCCTCAGGCTAACAATTTCCTTGGTGTTAGAGTATTTAAGGAGTGAGGTTGATATATAATTAGTGATTTAAAATACATGGTAAT
TTAGCAGACATTTCTTTTTGTTATTTTAAGTCTAAGTGTTAAATTTCTGATAGCATGTAAGGTATATTGCCTCCCTCTGTCCCTTCCTTCTTTC
CTTCTGCTTTTTCTTTGTTCCCCTCCCTTCCTTTTCCTTCCCTCCCTCTTTCCTTCCCTCCCTCTTCCCTTCCCTCTCCCTTCCCTTCCCTCTC
CCTTCCCTTCCCTCTCCCTTCCCTTCCCTCTCCCTTCCATTCCCTCTCCCTTCCCTTCCCTTTCCCATTTTCCCACTTTGCCTCATTCCCTCTT
TGCCTCTTTCCCTCTTTCCCTCTTTGCCTCTTTCCCTCTTTCCCTCTTTCTCTCTTTGCCTCTTTCCCTCTTTCCCTCTCTCTTTTTTCTTTCT
TTCTAACAAAACACAGAAAAGTCAATACAGTTTCTCATCTTTATCCACATGGAAATGATTTATGTTTTTGGAAGTTTATGATTTTGAGAGAAGA
GCCTATCCTGGGGGCAAACAGTAATCCTGCCAACCAGAATACCAAAGATTCTGAGAGGACTCAGGTCTTTTCTACTAATCTGCTGTGAGTCACT
TAGCCTCCATTTCTTCATAAGGAATGTTAACCACTTCATGAAAATAATACAGATTAACCAAGCCATAGCTCTAGAAACCAGACTCCACATGTTG
CTGGGCCTCACTAGCTGTCTGGGAGCTATAAGGCTGCCCTAGGAAATAATTGTGTCTCCCCAAGAAAGCCAAGTCCCTGACCAACGCATGAACC
ATTCTCAGAATCCAGCAACGGTTGTTTTATATTAAATGAATCTAATTACTTAGTAGAGTGAACCTGTAACTAATTTAAAGAAATACAGCAAGCA
TTGTTATCTTAGGTTGAACAAAACAGTAGCTTTAGGTTTCTATATATATTACATTTATTTCAATAGTTAATACTTTTCCAATCAATGCATGTGA
ACCATGATAGGGAGTTTCTATTTCTTGAAAGAGTTTGTGCCAGTGTCTTCTGGATAAGGAAAGGCATCATGGGAGGTAGGGCAAGGAAGCTGAC
AAAAAACATGTACAAGGGGAAATATTCAAGAAAAAATGTACACAACCACACAATCACATGCTGGCAGTAAAGGGGAAACATGGCCTTTCGTAAT
ACGGGAAACAGGAGAAAATGGGAGGCTAAAATGAAAAGAAAAAAACCTATGTCAGAGTAAGGAACTAGGACCTGAATTACACTCCAGAAGCTGA
CTAAGATAGAATGCTATATAAATTGCTCAAGAAAAAATGTACACAACCACACAATCACATGCTGGCAGTAAAGGGAAACATGGCCTTTCGTAAT
TCACATTATTTGGGGACAGGCTCTTGTAAATAGGTCACAACACATTTTATTTTATGCTTTTTTCCCCAAACATGAAATATCAGGAAATATAGTT
GCTGTTTGGTTTCAAGCTCTGATTGGAGACAGTTGTTGCATATGAATGGAGGTCAGATGAAGGGTGGTAGAAAATGACTATTTTAGCACTGGCA
CCCAGAAGGATGGGTCATAATTCAGAAGTGCCCCAAATCACACCTATCTGTAGAAGACTTCCTGCCTTTTTGAGGTGGTGGAATATGAACACCA
GTGCTTTCTGTGGTTCCTGATTTCTTCCTTCGTGACAACCTTTGTCAGTGTTTGATACAAGGTGCATGTATCGGTGTTTAATAAGAAGCAGAAA
CAGTGACATAGATGAGGGGTTTATTCCAGGGATTAGACCTCAGGCAAGTGTGGGATCTGGTGAAGAAGTCAATGGGAGTGTGTTGCATGCTGTC
TGATAATGGGCTGAACTCTCCGGGTCAGCACAGCCACCAGTAACAAAAGCTGGGCATGAAGTAGAGAAGAACAAAGAATTGGAATTAGGAGGAA
AACTGGAACCCACATCAGCCTTTTACCAACCTCAATGGGGGTGACTTCCAGAAGGATCTAGTGCCCTCTGCCATGGAGCTGATGCATGTACCTA
GCATAGGACTTGGCAAAGCTGAAGACAGAGCTTTGGGAGCTTCAACTACAGCTGCTGCGCTAAGTCCAGGTGAGTCACCACATCAGGGTAGTTG
TGTGCTTGCCATCATAGAGTTTGGTGCCCCCTTCAAATGGTTGCTGCTTCAATTCTGCTTTTCCAACCTCATGCAAATTTCCCCTAAGGCTAAC
CTTAACTCAGTGTGAATCAGGAGAGGAAAATTCTGGAAAATATAGCTCCTGATTTAGCCAATTTGATACTGTAGAAGCTACCAAAGTCCATCCC
TTGCCAACTTGGCATCCATGTCCACTTCTCTTAACTAAATATAACTTCCAAATGAAGACATTAGCAAAACGATGCTTCTATGTAGTATGATGCA
ACTATCAGTTCTACACATGAAAACACATTTAAAAGACGACTGGGTGCAGTGACTCACACCAGTTATCCCAGCACTTTAGGAGGCTGAGGTGGGT
GGATCACTTGAGGTCAGGAGTTCGAGACCAGCCTGGCCAACATGGTGAAACCTGTCTCTACGAAAAATACAAAAATTAGACAGGCATAGTGGTG
CATGCCTGTAATCCCAGCTACTCTAATCACTTGAACCCAAGAGGCAGAGGTTCCAGCGAGCCGAGATTGCACCACTGCACCACCCTGGCTGATA
GAGTGAGATGATGCCACACAAAAAAAAAAAAAAAAGATACATAGCCTCATAGGTTACTTTGTCCATTTTGGGATGATATTCAGTCTTTTTTTAG
CTAAGATCCACTTTCCCTTCGATGTCATGTCATTTGAATATACTGAGATATAATGATATTATGGGTTGAATTGTGTCTGTCAAAAAGCCATGTT
GAAGTGCTAAACCCTGAACCTTGTAAATATGATCTTATTTGGAAATACGGTCTTTGCAGATATAATCAAGAAGAGGTCATATTGGATTAAGATG
GGCTCTAATTCAGTGACTGGTGTACTTATAAGAGGAGGGGAATTTGAACACAGACACAAAGAATGTCACGTAACAATGGAGGCAGAGATTGGAC
AAAGGAACATCAAAGATTACCAGCAACCATCAGAGGCTACAAGAGACTCATTGAACAGATTCTCCCTCAGAGCTTCTAAAAAGGAAGTGACCCC
ACTATCACATTGATTTACGACTTTTCGCATACAGACTGCAGTAATAAATTTCTGTTGTTTTAGGCCACCCAGATTTTGTGGTACTTTGTTATAG
CAGCCCTAGGGAACTAATACAAAGACTATTATTACCCCATTTTATATTAGATGGTGGATGGGGTAGAAAAAAGAATTGGTTAATATACAACCAA
ATATATTTATATCAAAAAATAAATATTCATAATTCTTACCATCCTAATTTCTATAACTGATCTTGTGGTCATAGCTGGTATTTATAACTTCCTT
CTTTCACTACCCATTCCATATACTTGTGTCCTCAGCAAGCACATCACCTAGTCATGGTTTCTTGCCTGTTGGGGTGATGTCAGTTTTCATTTTT
GAGGGATCCAAGCTTTTGCAATTGAGCCTGCATTGGGTTATTTTCCACTTACTTTGTTCGTTACAGCAACAAAAGTAATTACAAGACTTGAGAG
TATAAGATATGATCTTGAATTCCAGGCACACTTTTTCTTACTCCTATAATCTGGTAGTAATCCAATTTCCCCAGTCATCCCAGCCAATACACCA
ATATCTGACTTTGCTTGTTGATTCTTTAGCATAAGGAGCCCAAAGTCGGCAGCAGGGAGTATCAACTTCCATCTTAATGGAATCATTGCTGTAT
CTCCTAGTAGAAGCATCTCTCCCTTGGGGACCAGAACTTCTACACCAGCAGAGTCTAAAGTTGAGAATAGAAAGAATAAATTTTGCTGTGGATC
ACTATGGGTAACAGTAAGAGGAGCCCTTTCCATTTTTCCCCCTTGATTCCTGGATCTATGAAATCTGGATAAGAGAGAAACAGCAACATGCATT
GATTGATCATTTAGAGAATTTACTGCATCTTTCAGGACATTATCCTAGCCCTGCAAAGTGTTTCCATCCATCTAGCACAGTAAATAGTTAAAAG
AAGAATTCCACCTTTCTACTGCTTTAGAGTGATGAATAACAAGTTAAGACTAATGAATTCCATGAGCATGAGTCTGTTATTCTTCATTTGCTGC
AAAGTAAGTTCCTTGGTCAAAAGCAAAGCACTGTGGAATAACATGAAGGGAAATAAGATATAAGAATTCTTTAAGTCCACAGATACTGATTTTG
GCAAAGGTATTACACGTAGGGAAGACAAATCCCTATAAAGAGTAAGTTTCTATGGAGCAAGAACAAAATACTGCCCTTTCAGGCCTTGTGAGTA
GAAGTTCATGTTGCTGAATTCATGCATACCTCCATGTCTACCATCATGGCTATTTTGTTCACGAGCTCATTGGTCAAGGAACAGAAGAAACTGG
AAAGAAAAACCTGATAGAGGTCTGCAGAATGTGTTCTCTTGTCCATTATTAAGATTCTTACATGCTGAGGTTGAGCTTTGATCAACATTCACAT
GGGCCCATTTAGGAAAGTCTATCCATTTACTTCTTCCCTAGATTTTCCTGTCACTAATTTTCCAGTCATGTTTCTTCTPAGTCCCTTATTATCC
GGGCCCATTTAGGAAAGGTCTATCCATTTACTTCTTCCCTAGATTTTCCTGCACTAATTTTCCAGTCATGTTTCTTCTAAGTCCCTTATTATCC
GAAGTTCTTATTACTGGGAAAATTTTTCTTCCCCACACCACCAGTATACGGCCACAATGATTTAGTCATTCGGTTTTGGATGATACCAGAGTAT
CACACAAAAATTTCTGTAAACAATGCCAAATATTTTTCAGTCCACTGGTCACTGAGTGCTTCCCATGAGGCCATGGGTGTAAGATTCTGAAGCT
ATTGTTGCACAAATAGACACCATGGGTAACTGGGTCACTTGCTTATTTAATATACTTGTGCTTGATCTCATATATTCTACTTCCATTTGATCAT
TGAATGAACAACTTAGAGGCTTCCATTTGATCATTCATGCACAACTTAGAGGCTTGATGGATCAACAACACTTAGTTCATCATGGGCAGTTCAG
GTTGCATTATAACTTGATGGCCCATGATCAAGCTTCCAGTTTCAAACTGGTATTCTAAAGAAGAATTTTGAAGAGAAAAGAAGAATATCTGAAT
TAAAAGAAGAATTTTTCTGAAGAAGTTGTTATAGTTTTGCTCCAGAATTCTAAATGTCTGCTCTATGATTCATCTATCAGGTTAATGGCTTCAT
GCAAGATTCCTGCCTAACAGAGACATTTTTAGCACCATGGAATCTACTGGATCTAGATGTAGGAGCAGTTTGTATGGCAGCCTTGTATTGTAGA
GGCTTTTCTTGTTTTGGACCTCACACAAAACTAATGACCTTACATCTTACTTGGTAAATGGATTGTAGTAGCATGCCCAATGAAATATATATTC
TCTTCAAAATCCACAAAGGCCCATTAGACATTATCCCTCTTTCTTAATAGTAGGAGAGGTCAGATATAGCAACCTGTCATCATGTTCATGAAAG
GAATATCTTGGAATGCTCAAAACAAGTAGATCACTACAAATTTCAGTAACATTCAGTGGATATAAAGTGAATTTTGTGGAATTTATCACTCTCT
GGTATGCAAGTATCTTAACATCATATCTAGAACTAGTTACTTTATCCTATTTACCAGGTACAGTAATCATGATATAATATCATTAATATAATGT
GCCTGCATGGTATCTGATGGAATGGATAGACAATTATGGTCCCTGTGGACTAGTTACAACACAGGGCTGTAAAGTTGAAATATAGCCTGAAGTA
GGACAGTGGAGGTACATTAGTAACCCTGTTGGCTGGAAGCAAATTGCTTCTGATAGTATATACCAGAGGTTAGCAAACATTTTCTGTAAAAAGC
TAGAAAATAAATATTTTAGAATTTGGAGGACATATGGTCTCTGCCATAATTACTGAAATCTGCTGTTGCAGCATAAGAGCAGCCATACCTAATA
TGTAAATAGAGAAATGTAACTGTGTTCTAATAAAACTTTATTTATAAAAATCCAGTAGTCTGAATTTGGCCCATGAGCTGTAATTTGCTAATTT
CTGGAATTTATTAACAGGTATAGAGAAATAAGCATTTGCCAGCTCAGTGGCTCCTGACCAGGTGCCAGGAGCCCTATTGATTTGTTCCATCAAT
GAAACTACAGTTGGAACAACAGCTGCAATTGGAGCCAACATCTGATTGAGATATTAAAAATTCACTGTCATTCTTCAAAATTCATCTGTGTTCT
GCATAGGATAAATCAGCAAAATAAATGGGGAATTTGCTAGGAATCACCATCCTTGCATTTTGTTTCAGTTCCTGTTGGTGGCTCTAATCTTCAT
CAATAACAATTGTTTTGTTCACTAATCTGTTTTAGGCAGGTTCAATAGGGACAGTTCATCTCTGATAAACTCATGCAGATAAGAAATGTCTTCT
GGGAAGGCTTGACTGGGGCTGGAGAACGTACTTCCAAGATAGCTTGCTCAAATGACTGGCAAGTGCTTCTTATAGTTGACTTGGAACTCATCCA
GGGTTATCCACTGGATACCTTGGATCCTCTCCACATGGACCTCTTCATAGGGATGCTTGGGCTTCCTGAAAACATGGCAGCTAGTTTCCAAGAA
CAAGGATTCCAAAAGGCATGGATGAAAGTTGTAAGTCTTCTAAATCACTTAAATTCCAAAGTTCTGGATCACTTCCGCCAAATTCTATTTATTA
AATGAATCAGTAATGCAAGCCAGATCCAAGGGTTGGGTAATTAGACTTCACTTCTCAATGCATAGAGTAGCAAAGAACTTGTGGCCATCTTTTA
ATCCACGATAGTTCACTTGCACAACAAAGCCAAACTAGGAGACCGACAAAGGTTACTACCCCAGAGGAGAGAAATTTTTGTAAGAAAATAATAT
CGAATTTCACAGATCTACTAAACCACTGGGGGCTCCACTGAGCAGTATTTCAAATGGATTACACTCTACCAAGTCAGGAAGATAAGTAAGAGAG
GTAGAGAAAAAGGCCCAAAGAAAAGAACAGAACACAAGTATTTATTCATTAGTCCTTATTCCTTATATATGTCAATTTCAAGGAAGTCATCAGA
AAGACATTTCCTTTCTCCTAATCTGGACATGAGTCTCTGGGATTCTCTTGAAAGTCATCTTCCCAATGCTAGGCAGCACTGCACCTTAGCATTT
ATCCACGATAGTTCACTTTGCACAACAAAGCCAAAACTAGGAGACTGACAAAGGTTACTACCCCCAGAGGAGAAGTTTTGTAAGAAAATAATAT
TGAGGGGCCCATAGCCTCCCATGTATCTATTTAGACTATAGGTTAAAGACATACTTGGTCTTCCCTATAACACTTACGTCAGTGTCTTGGTTGT
GGTATTGCTCAGTATTTTATTATTGACTCATCAGAGCATTTTTACTCCATTGGCTTTTGACATCTAAAACAGTTGTCAACTTATAAAACTCCAA
AATATTTAACAATCAGCTCTCATAAGCTGGTGTGTGGGATGGTATCAAAAAGCTCTCCTCTACCTCGTAAGGTTGTCACTGCAGACAGTACTTC
AAGGATTTTTTTTTTTTTTATCTTTAGCTAATTTAAGCTGTCACTCCTTATTTTGGATGGGAGGGAGAAAAATAGCTAATTGGAAGCAGAGGAA
AAAATAGTTTCAGGATCAATGGTGGAAATAGCAGCAAAATAATTATTATTACAGTCGTAATGCTCTGCATTTATATGGGGTTTTTTTCCAAAGG
TCTCAGAACACTTTCCTGAATATCACCTACAAATTCAAAAACAATTATGAAGAAGTCAGAGTAAAGCATAATTATACCAGAAATTTACTCTGAA
ATGTAATTCAATACTCTAATACAATTTTACTCTCAAATATAATTCAGTGAAAAGTAGACTTATTGTAAACTGAATTATAGACATACATTTGGAT
TTTTTTTTTTTTTTTTTTTTTTTTTTAAGACGGAGTCTCGCTCTGTTACCAAGGCTGGAGTGCAGTGGCGCGATCTCGGCTCACTGCAAGCTCC
ACCTCCCGGGTTCACGCCATTCTCCTGCGTCAG
HUMAN SEQUENCE-mRNA
CCACGCGTCCGCAATGAAGCCGAGTGAATGGGGGCTGAATGTGCGAGTCCATAGCTGAAGAGGAGCGCCAGATGGTGGAGGAATACACTTATTT
ATGAAACTGTCTTGAGTTCTTCTTGAATTGCCAGTTTTCAGCCTCCTCATGCCTCCGTCTCCTTTAGACGAGAGGGTAGTAGTGGCACTATCTA
GGCCCGTCCGACCTCAGGATCTCAACCTTTGTTTAGACTCTAGTTACCTTGGCTCTGCCAACCCAGGCAGTAACAGCCACCCTCCTGTCATCGC
CACCACCGTTGTGTCCCTCAAGGCTGCGAATCTGACGTATATGCCCTCATCCAGCGGCTCTGCCCGCTCGCTGAATTGTGGATGCAGCAGTGCC
AGCTGCTGCACTCTGGCAACCTACGACAAGGACAATCAGGCCCAAACCCAAGCCATTGCCGCTGGCACCACCACCACTGCCATCGGAACCTCTA
CCACCTGCCCTGCTAACCAGATGGTCAACAATAATGAGAATACAGGCTCTCTAAGTCCATCAAGTGGGTGGGCAGCCTGTGTCAGGGACCCCCC
CCACCTGCCCTGCTAACCAGATGGTCAACAATAATGAGAATACAGGCTCTCTAAGTCCATCAAGTGGGGTGGGCAGCCCTGTGTGCAGGGACCC
AGCTGCTGCACTGTGGCAACCTACGACAAGGACAATCAGGCCCAAACCCAAGCCATTGCCGCTGGCACCACCACCACTGCCATCGGAACCTCTA
GACTGCAGCAGGGCAAGATCACTCTCCTAGACTTGATTTCCTGTAGGGAAGGCAAGGACTCTTTCAAGAGGATCTTTTCCAAAGAAATTATAGT
TTATGATGAGAATACCAATGAACCAAGCCGAGTGATGCCCTCCCAGCCACTTCACATAGTCCTCGAGTCCCTGAAGAGAGAAGGCAAAGAACCT
CTGGTGTTGAAAGGTGGACTTAGTAGTTTTAAGCAGAACCATGAAAACCTCTGTGACAACTCCCTCCAGCTCCAAGAGTGCCGGGAGGTGGGGG
GCGGCGCATCCGCGGCCTCGAGCTTGCTACCTCAGCCCATCCCCACCACCCCTGACATCGAGTACGCTGAGCTCACCCCCATCTTGCCCTTCCT
GTTCCTTGGCAATGAGCAGGATGCTCAGGACCTGGACACCATGCAGCGGCTGAACATCGGCTACGTCATCAACGTCACCACTCATCTTCCCCTC
TACCACTATGAGAAAGGCCTGTTCAACTACAAGCGGCTGCCAGCCACTGACAGCAACAAGCAGAACCTGCGGCAGTACTTTGAAGAGGCTTTTG
AGTTCATTGAGGAAGCTCACCAGTGTGGGAAGGGGCTTCTCATCCACTGCCAGGCTGGGGTGTCCCGCTCCGCCACCATCGTCATCGCTTACTT
GATGAAGCACACTCGGATGACCATGACTGATGCTTATAAATTTGTCAAAGGCAAACGACCAATTATCTCCCCAAACCTTAACTTCATGGGGCAG
TTGCTAGAGTTCGAGGAAGACCTAAACAACGGTGTGACACCGAGAATCCTTACACCAAAGCTGATGGGCGTGGAGACGGTTGTGTGACAATGGT
CTGGATGGAAAGGATTGCTGCTCTCCATTAGGAGACAATGAGGAAGGAGGATGGATTCTGGTTTTTTTTCTTTCTTTTTTTTTTGTAGTTGGGA
GTAAAGTTTGTGAATGGAAACAAACTTGGTTAAACACTTTATTTTTAACAAGTGTAAGAAGACTATACTTTTGATGCCATTGAGATTCACCTTC
CACAAACTGGCCAAATTAAGGAGGTTAAAGAAGTAATTTTTTTTAAGCCCAACCATTAAAAATTTAATACAACTTGGTTTCTCCCCCTTTTTCC
TTTAAAGCTANTTTGTAAAAGTTTATGAG
HUMAN SEQUENCE-CODING
ATGCCTCCGTCTCCTTTAGACGACAGGGTAGTAGTGGCACTATCTAGGCCCGTCCGACCTCAGGATCTCAACCTTTGTTTAGACTCTAGTTACC
TTGGCTCTGCCAACCCAGCCAGTAACAGCCACCCTCCTGTCATCGCCACCACCGTTGTGTCCCTCAAGGCTGCGAATCTGACGTATATGCCCTC
ATCCAGCGGCTCTGCCCGCTCGCTGAATTGTGGATGCAGCAGTGCCAGCTGCTGCACTGTGGCAACCTACGACAAGGACAATCAGGCCCAAACC
CAAGCCATTGCCGCTGGCACCACCACCACTGCCATCGGAACCTCTACCACCTGCCCTGCTAACCAGATGGTCAACAATAATGAGAATACAGGCT
CTCTAAGTCCATCAAGTGGGGTGGGCAGCCCTGTGTCAGGGACCCCCAAGCAGCTAGCCAGCATCAAAATAATCTACCCCAATGACTTGGCAAA
GAAGATGACCAAATGCAGCAAGAGTCACCTGCCGAGTCAGGGCCCTGTCATCATTGACTGCAGGCCCTTCATGGAGTACAACAAGAGTCACATC
CAAGGAGCTGTCCACATTAACTCTGCCGATAAGATCAGCCCGCGGAGACTGCAGCAGGGCAAGATCACTGTCCTAGACTTGATTTCCTGTAGGG
AAGGCAAGGACTCTTTCAAGAGGATCTTTTCCAAAGAAATTATAGTTTATGATGAGAATACCAATGAACCAAGCCGAGTGATGCCCTCCCAGCC
ACTTCACATAGTCCTCGACTCCCTGAAGAGAGAAGGCAAAGAACCTCTGGTGTTGAAAGGTGGACTTAGTAGTTTTAAGCAGAACCATGAAAAC
CTCTGTGACAACTCCCTCCAGCTCCAAGAGTGCCGGGAGGTGGGGGGCGGCCCATCCGCGGCCTCGAGCTTGCTACCTCAGCCCATCCCCACCA
CCCCTGACATCGAGAACGCTGAGCTCACCCCCATCTTGCCCTTCCTGTTCCTTGGCAATGAGCAGGATGCTCAGGACCTGGACACCATGCAGCG
GCTGAACATCGGCTACGTCATCAACGTCACCACTCATCTTCCCCTCTACCACTATGAGAAAGGCCTGTTCAACTACAAGCGGCTGCCAGCCACT
GACAGCAACAAGCAGAACCTGCGGCAGTACTTTGAAGAGGCTTTTGAGTTCATTGAGGAAGCTCACCAGTGTGGGAAGGGGCTTCTCATCCACT
GCCAGGCTGGGGTGTCCCCCTCCGCCACCATCGTCATCGCTTACTTGATGAAGCACACTCGATGACCATGACTGATGCTTATAAATTTAGTCAA
AGGCAAACGACCAATGTATCTCCCCAAACCTTAACTTCATGGGGCAGTTGCTAGAGTTCGAGAAGACCTAAACAACGGTGTGACACCGAGAATC
CTTACACCAAAGCTGATGGGCGTGGAGACGGTTGTGTGA

TABLE 33
MOUSE NOMENCLATURE
ICSGNM Fli1
Celera mCG14021
HUMAN NOMENCLATURE
HGNC FLI1
Calera hCG39646
MOUSE SEQUENCE-GENOMIC
CTGTGTGTGTGCAGCCTGACATTAGAGGGGACAAACTGAAAATGGCTGCAGAGCGTATGATCCCTGCAGTCAGTCAACTGGAGTTGTCATTCTG
CAGCTGAGGAACCCCAGGCAAGTCCTCGGTCTCTTTAAGGTTTGGTTTCCGTCTTGGTACCATGAGGACCCAACTTACATTTTAAAGGGTTATG
CTTACAAAATTGTCTTGGGAACATCAAACAATAGTTATTCTAGCTTTACTTTTTGACACAGATCTTGCTAAGTAACCTAGACTGGGTCCTCAAA
CTCTGGATCTGTCTAGCCTTAAGCATCCCCAACCGTTAGGCTACAGGTGTGAGCCACCAGGCCTGGCTATTTAACCTGTCTAGTTTAAAAGTGT
GAGTTGGGGGAGTCGGGGGTGGGGGAGTACACACCTTTAATCCCAACACTTGGGAATCAGAAGTCGAAGGATCTCTGTCAGTTTAACGCCAGAC
TGCTCTACAAGTTCCAGGACAGCCAGGGCTACAGAGACAGTCTCTGTCTTGAAAACCAAACCAACCAACCCACCAACCAACCGAACCAACCAAC
CAACCAACCAGCCAACCAACCAACCAACCAACCAAACAAACAAACAAACAAACAAACAAAAAATGGTCCTTGTCTGTTTGGTGCTTGGCTTTAT
GGCACCGGAGATGAACATAGGCTTTTACTAACCTAGGTGAGGACTGTGTTTCTGAGCTACAACAGCTGTTGTCACCTGATGTCCATTGTGTTGT
CCTTTCCAGAGAGTAATTCTACAATATAGTGGTTAGAGGAGTTTATCCCAACATTCATTCCCTTTCACTGTGTGAACCTGGAATAGTTATTCTC
TGTGCCTCAGTTTCTCCATCTGGTCCATCCTGGTAAAAACCACAGTCAACGGATTTTCAATCACCCTTTGAGTATGGATTTTCTAGCACACACA
CACACACACACACACACACACACACACACACACACACACACACACAAATCACTCCCAAACTCACAGAGGTCCTTCAGCTTCTGGTTTCCAAGTG
TTGGGGTTAAAGGTGTGCATCACCACTACCTGGCTCACACTTTTAAGTTAGAAAGGTTAAATAGCCATGTGTGGTGGCTCACACCTATAGCCTA
GGCTTGAACGAACTACAGTGGCCCCCATCCACTCTATTTAATGGTTTGCTGTTATAATCTGGAGGTCAGTTCTGGCTGGTGGCTGGTGGCTGAT
GACCGTAGGTCCTCTCTGTGTGTGCATGTGGTCCTTTCCATGCAGATTTAGTAACTTCTTTGGACTTCCTCATAACATGATGCTGATTTGTAAC
GACTAATATGAAGTGGGTGGAGGCATTAGTACTTTTTATCATCTAGCCTCTGAGGTCAAGCAAGAGAACTTCTATGACCCTGAATTTGTTGACG
CAACCATGAAATTCCATCGGGCTTCAGGGGTATAATGGTTAACTTCCATTGTAATTTGGATGATTTATAGTCACCATGGGCGTGTGTCTCGGGG
TGAGTCTGTGGAGATATTPCGGAAAAGGTCTGACTGATGAAAAATGCTTTAACTTGAATATGAATAGCACCATCTCACGGGCCGGGATCCGACT
GGAGAAAGCGACCTCCATGCTAGCATTTGTCTTTCTTGGCAACGTGACCATCAATGCAGTGTGACCAGCCACCTCATTTCTACTGCCACCATAC
CCTGCCCAGGATGATGGATGGGATCACCTTAAACTACAAATCAACACAAACACTTCCCTCTTTAATTTGCCTCCTGCCAGGTATTTTCGTAACA
GAAATGAGAAAAGTTGCTGACAAAAGGGAAGAGAAATAGAATCTGGCTCTTAATGGACGAGATAAGACTGTACATATGGAGATAGCCGPTAAGG
GGAACTGGAATTAACTTTAGTAACTGGTGACTGACAGGTGTTTCACATCATCCTCCTTATCCTACTTTCCATTAAATTATCTTCTCATTCCTTC
TTCGCGAATCAGAAGGCTCGCCCGAGAGCACAGCGGGGTCCCTCTCCGAAGTTCATCGCGCCCTCGAGACTCCCACTCTAGGCTGATGTTTCTC
CCTGTGCAGAATGGTTCCCAAAGCAATGAAGGCAGCAAGCTCTGCTCCCAAGAAAGCTGTCAGCATTTTGTATGCCCTCAGGTTGACAAGAGAA
GCAACTTCATGTCGTTATGAAAAGAATGCAGCAAAATGCCAAAGCTCCGGTGTTGCCCACGGTGACCCTGAGGAGCGGATATGCCAGAGATTAG
CTTTGCAGAGCACTAGTCTGGGAGCCGCTCTGTGGTCCCCCTGAACATGAGAAGTGACGGGAAACTTGACATTCCTTTTCCCCATCACCTCCAG
CAACATTCATCAGCTACTGAAATTGTCCTTTCAAGGTCCAGGGCAGGACAAAAGCAAAACAGGCATGCGTCTTTCGGAAATTACTGCCTCTTTC
CCCCACATCTTGTGCCCTTCTCTCAGAAAAGAAAACACAGCTTAATAGGGCTGAGAATGAGCAGGAAATCCAGGACTTCTCTTCTGTCTTGTTT
CAATCTCCCAGCTCATAGAAGGATGTGTGAGGAAAATTTCTTTGGAATATTCCTGAATAGAGTGTAGAGATAGATCGCCAGGCACTCTGGATGT
CTGTCTTTGCCTCTCTGGATGTTTTTCCGTGTCTGTTTTAGCTAAGCTGTTTAAGAGATGGACTTGGTGGCTCTTCCTTCATCCTATGACAGTG
TTTATTATTCTTCATCTGACATGGAAAGCACCTACTAACCTAACTAAGGACAACAAGGTAGAAGATATATTTGTGAATTTATAAAACGAATAAA
TGGCTATATGTATATATGTATGAACACACACACACTCATATGTGACAAATTCAAAATAAGTGAATTTTAGCTAATGAATCAGATCCCTGCATCC
CTTTTTCTGTCACTTGCCAAATCCGTATTATGTAAACACAGTATATGCAAGCAGGCACTACCACTACTGTATCCATTACTTTGCCCATGCATCC
CTCCTACGTTGGTTGTGGAGCCACTGAAAGAGCCTTGTCTCTGCCACCTACAACCCCAAAATCAAGCTAGAAAGGGGAAGAAGTCAGGGCCTAC
GGGGAACAGCATCCATCACTTGAAATGAAGATTGGAAATGCGGAATAGTAAAGCACTCTGCTTAGAAGACCAGCATATTCTTGTTTCAGAATCT
AACAGTTATCACCAACTGCTAGAGTCTCTAACCTTTGAAATGATCTCTTATTTTATGTCAGTTTTTTTAAAACAATACCAGTCGTTATTTCCAA
TGGCTCCTGGCCATGTAAGACTCACTGAGCATCCGCCAAGTAGGATTTATTGGGTGGAGCGTACTTCTTCCCTGCTTCAGACAAAGCCAAGGCA
AAGAGTTTGCCAAGGGCTTCTGGTCAGCATTGGGAGAGCTAAGGCATGAAAATGGAAGGCCCAAGCCAGTCAGAAGGAGGGGTGGAAAGGAGGG
AGATAGGAAGATAGGGAGGGAGGAGGGAGAGGAGGAGGGAGGGNNNNNNNNNNNNNNNNNNNNAGAGACAGAGAGAAAGAGAGAGAGAGAACTA
TTCATAAGGGAACGTGCTGTCAGAGAAACAGCATCGACCATGGGAAAAGAGGGCAAAAGATAAAATAGTAGACTTTAAGATGATGCAGTAGACT
TACAGATGACCATGTCCTTTGCTCAGGAGAGTAGCCATTGGCTGAGAGGAGGACAAGTCATCCCAGGAGCTACTTGATTCCCTCTTGCAATGTT
TCTCTACAAGGACTATAAATGATATCATAAGTATTGTGGGGAGTCCTGAAAAATGGCCACCCATGGTCAGAGAGCTACATCAGATTCGGATTTA
GCTGCTTGCTTCCCAAGCAGGTCTACTGAGCTTTCTTCGAACACCTGCTACCTAATCCATGCATACAGCAGACACTCATCGAGCAAACGAGTGT
CTAACACGCTGGGCCCTCGACACCACTCAGACCCCACCCTGGTAATGAGGCAAAGGATGCCTCCTCCCACGTACTTCCATCCTAATCCTACATA
ATAGTCAAAGGAGTTCTCATTCACATGTCTCTTTGTCTCCGTCTCCCCCCAGCCCCCGACTCATTCATTCGGAGACATGTGTTCATTACCTACT
AGCTGCCAGATGCTATTCTAGGCAAAGGACTTGCATGGAATGATGCCATGTCCCCTAAGGGTTTAAATGTCATACTCAGGAGATTAACAAGCTC
ATGACTCCATGGGCAGTGATGGTTCATGATTGGGAGTCTGGTTTGACGTCACTATGGGAATGCAAGATGACTGCAGCTGTGTGTGTGTGTGGGG
GGGGGGGGGCTGTTGTGATGCAGGAATCCTAGAAAGGAATCGGCTTGTTCTAACTGAACATTTCAGAGCGTGCTTCTCCATGCAGGGACACCGT
GCCCAGTACTGAGGCATTGTGGTTAAAGTAAAATAGAAGGGACACTGAGGATACATAGGGATTCTCCTAGAATTGCCAACAGAGTCTCTAAGGC
CTGACGTATTCCTCATTCTCAGGGGAGGAATCTAGCCAGGTTCTCTGGAGCTAGCTGCCCATCTGAATTAGGGATTCCAGAACAGCAGGACTGT
TAGGAGCCAGAGGTTGATGACTAGATTATAGATAGGCTCTTTAGGAGAATTATCTAACATGCTTATGGCGGCTTGGAACTTTCCAAATGCCCAT
TTGCAGACTGTAGGACCAAGGAAGTTAGTACTGTGGCTGAGAAAGATGGTCGAGTGCTGCTATTGTAAGCCTGCGGCTACTCCCAGCCCAGCAG
TCCCAAGGCCACATCCTGGGCCCTGATGTCTAAGGGCAGCAAGGGAAGGCTGTCCCAGATATGACAATGATGGTGATGGTGGTGGTGGTGGTGG
TGGTGGTGATGGGGAAGAGGAGGAGGAACAGCAGAAGCAGCAGCAGCCAATCAAAGTGGCCTCTTTTCTTGAACATTTAGGGAAAAGGCACAGG
GAAGAAACAAGCATTGCTCAGGTTCCATCCGGTCCGTCCCTGTCTCATCATCTCCACCCCAAACATTTTCAGGCTATCTCTTGGTACTGCCTTT
AAAACTAAAGATGTAATTATTTACTCTGTCCCAAGAACACTGAATAGACAATTAGGCAGTTTACCCAGATGATGTCATTTTATCTTCATTTACG
CCCCACCAGGTTAGGGCTTGTTGGCATCCCCAAGCTACAGATGAGAAAACGGAGCTCCTACAGTATAAAGTCATTGCACTGGGAACTGTAGGAA
CAGACGATGAAGTTTGGGTTGGCACTTAGAGCCTCCCTGGCTCCAGATTGTGCTTGAAGTTCTAAAACCCACAAAAATCACAGTTTCAAAGGGC
CAATTGTCTAACTGGCAAAACAAACCTTCTCAGTGACTGCTACTGGGTCTGTGCCCAAGCACAGCGCATGGATGGAAACCCCACATCATCTTCA
TTTTCTGTGTGCATGAACAAGTTACTTCGCTTCTCCGAATATCTGTTTTCTTGCCTAAAAAAAAAAGAGGTGAAAACACCTCTCATATCACTTG
GATGGATGAATTAGATGTTTCGTGTAAACATAATTTCACTACTAGTAAGTGCTTTGCTAAATTGTCTACTGTTTCTATTTATTTCTATAATTTA
AGACCCAGTTTATTTGAAGTGATATGGGAACGTTACCTTATCTGTACTAGGAAAGCAAGCTAGTTTTCATCGGAGCCTATGTATCAGTCATTGA
TAGTATACATCTCCTAGCTCCAGCAGAGGATGGGGGGAAGTGTTGCCCTAGGCCTTAGCAGTCCACTGCTTTGTCTCAGAGCTCAGCCCGCTAA
CTCAAGCAGTCCCTCTGAAAGGACTCTAAGGAAAGGGGTCGCCACAGAGTACCACTGTGCTAGAATATTTGACAACTTGGGGTAGGGAACATTG
TACCACCTTCTAGGTCTGCAGCTCAGGGGTGAGGCACCAAGGCATCCTGACTAGGGGATGCAAAGGTGCTTCTAGTGAACCAACTGTTGAGCTT
GTTCTGGGCTCCCTTGTAGTGATCCCTACTGGGGTGCTAAGAAATCTTATAGGTGGGCATTGTACAGAGGTAAAGAACCTAGTTTTAAGCAGCA
TGGAACCAACAGGTCTTTCCAAGAATAGCCTGTGTTTTCTGAATGAAATCATCATTGCCCCACGTTCATGGGAAATGCAAACTCATTAGAGACC
AAGAACAGTGGTGAAGGTGAATCCCCCATTGGTGTTCCTTTTATATTTGGTCTGCCGTGCCAAAGACGGGTACTTCTCAATATCTCGCCACTAG
AGCTGCTTTGACAGCAGCATAATGCTCATTCCTATAAAGAGTTTGAAGTACCCGTTAATAGCTTTGAGTTTAAGCATTAGGAATTTTAAGGTTC
AAGTATAGAGCTTTGAGAGCACACATTAAGATAGCAACATCAGCTCATAAAGCTACAAGAGAGAGGCAGTGCTCCCTGGCACACTCACTCTCAC
CTGTCTGCCGCAGCTGCACCTTGGTACTGGCTTCTGCATCTCATTGTCCATGGTCTTCTCAGTCCACAGAAGTGACACAAAGGCCTGCCACCAC
TGCCCAGCACCCATCCCCTCCTCTGCACTCTCCCACTCTCCTGTTTCCATCCTGGATGTCTGTTATCTCCAAAGCAGTTTTGAAAGGGTCCTCA
CTCTCGCCCGGAAGCAGCAGGGCACCCAGGGCATGGTCTCAGGCTTGAATGACAAAAGGGGTCCTATTTCCTGGCAGGTGTGGCAAGAATGTCC
TCCTAACTAGGTTGATTTTCAGATTCAGTCCAAAAGCCAAACAGGGGTCAAACCTTTTTCTTCGGTTCTTTCTTTCCAGCCTCACTCATCTACA
TCCTAACTAGGTTGATTTTCAGATTCAGTCCAAAAGCCAAACAGGAAACAAACCTTTTTCTTCGGTTCTTTCTTTCCAGCCTCACTCATCTACA
GAGTCTGCTTTCCACACTACTATGTGGGCAGAAAGCCCTTTGCTATCAATGATTGTAAATACAATAAATCCCCCAACTGTGTGTGTGCACATAA
AGGATGCATGCATGCTGTATGGAGTGTGTGTGTGTGTGTGTGTGTGTGTGTGTGTGTGTGTGTGTGTGTGTGTGTGTATGTGTGTGTGTTTTAA
AAGATCCAGAAATTCAGGACATTTTTCAGGGAAGGTTTTCATTGAGTTAAAAAGCACCCCAGTGAACCGTTAGAACTGTACAGAGGTTGTAGTG
GCAACTCGCAAATGGTTTGACAGATAGGGCAAGGCTGGGTAATTGAAAACCAAGTGACTGAGTGTAGGTTTTTCCATGGTAGAAATACAGCAAG
TTCTTGGGGGAAGATTGACGGGTACAAGCTGTATTCACAGCCTTTGGAGGAATGGGATCTGTCTGAGATTAGGCACAGGAGATTGGGGAAGGGG
TAAGAGCAGTGTGAGCACAGATCTATCAGTGTACGTGCCCTGGCCGCGTCAGQGGTCAGAGGTGTGGGGCATTTGGGGATGAGCTCGCCATACT
CTCTTCCTGTGATGCTGCCACTTGACTAAATGACCTTTGATTGGCTCTTTGTTTTAAATAAAGTGTTTATCCTAAAATGGGTTCTAAGGGGATC
CATCCCTTCTGCTCTTGTGCCAGCCCCATGCTGTGCTGCTGTTTCTTCTGCCTGCAAATCTCACTTCCTTAACAAGCCTCCAAGTCTTAGCTCT
CCCATTTGTTCCCTGGTCCAACCTCTTTGCTCTGCGTGCTGTGCATGCTGCACCATCTGTATGTCCTCCTTGGGCTCAGCATTCCTGCCTTTTG
CTGCTTTTGACCTCCTGGPTGGATGGCAGTGATGACAAGCGCCTTCCCTTCCCCCACAGCATCCCCATGTCCATCTCTGCCTCCCAGATCCCCT
CAACTCTGCCATTGTATGATTTTGCTGGAACTCTCAAGACTGACTCATGACTAACTAGAGGCATAGTCTGTTCTCTGCCGCAGAAATCTGCTTA
GATGACATCTCTTTCAACAACAGATCTGGGGCTTGTTCTGCATTTTTTTTTTTTTTGTCAAAGAGACTCTGTGAATCTTTGGTGACACCCCATA
TAATGTCAAAGATTTATGCTCAGCCTTCTGAACAGCCATAACTTCACAGTTTTCAACTTGGAGTTAAACACTAAGTGTCTATGTAAAAGTTTAA
GTTTGTTTGAGACACAAAGCCAGAGCAGAACCTTAACCGAAATGAAGAGTGGATCTAAGAACCAAAATACAAGTAGACCAGAGCCACAGCCTTC
CCCTGACCCTGCCCAGGGCAGCAGGTGGAAGCCAGAAGGGCTCCAGCAGCCTTTAGGTCTGAATGAAGGACGTCCAGACATAGCGAGGGTATTT
GCTTTAGAGAAGGCATTCCAGGCAGAGATATGAATTATGTAGGGAGAGTAGGCAAACCTCAAGATTTAAAAGGGTCTCATCATCTATCATTCTG
TTCTTAAGTAAAGGCAGGGTTGCCATAGCAACACACCACAGGCTGAGTGGCTTAAACAACATAAATTTATTTGCTCACAGTTCTGAAAACTGGA
AACCCAAGGTCAGAGTGCCAGCAGGGCTGGCTTCCTCTGAGGCTTTCTGCTCTGGCTTACAGATAATCTGTCTCCCTACATGCCTTACATGGTC
AGTCCTTGGCACACAGCCACCCTTAGGGTCTTTGTGTGTCCTTATCTCTTATTATAATGAGTCAGTTGAGTTTAGAGCCCATCTTCACATTCTA
ATTTCAAATTAATCATATCCTTAAAGGTCTTATTGCCAAGTGGAATCACATTCTAAGTTACAGGTGGTTAGGGATTTGATATATAGATTTCAGA
GGAAGCGGAATTCAGCATAGAACAAGAACCCAACAGAACCCTTGTTTGAACACAAATGCTCATCACTGCTAGTCTTTCTGGAGTTTTGTGCTGT
GCCTAGTGTTGCTATAGCTATTTCCCATTGATTAGTCCATCTTTCCTTCACAGTCGGGTTACATGGAATAGATTATCTACATGATGAGACGATT
ATAGATGGCTGCCATTTCTTCTGAACATGGTCAGCTTGCTTTTGTGTACTGCGTGGCCCTGGGCAATACTGTCCTCCAGCCTTGAAATCCCTGC
TGCTAATGTGATTTGGAATAGTGGGGGGGCGGGGGGATCTTATCTCCATGCTTTGATCGAACAGACAGTCTCTGGAAAGGAGTAGACTATGTTT
CTCAACCCATTACCAAAGCCTCCCCTGAGTCAGAAGATGGTACAGTATGAATTGGTAAACCATGGTCTCTTACAGAGACCTTACACTAAACTGC
CTTTTCTATATATCAGAGCTTCCTCCTGAATTATTTGGCTGATCATCACACTAAGATCTGCAAACATACTCATTCCCGCTAAAGGCCAGTCCTC
AGCACACCACTGTTCACTCTTACCAAACACTTGAACTACAGTTGCTAATTTAATTCACCTAGTAAGCCAGAAGGGAAAAAAAAAACAAACAAAA
CTATTCATCCTCACTGACTGGGTATACTGAAATCGACCTAGCTGAAGTTCTGGATTCCCTGATTCCCTTACATAGCTTAGGTCTAGACAAGCTC
TCCGGTGGCTCTCCATGACCTCACCTGTGGGATAATTCTCATTTTCATCTCTCCTACCIATGTGGCTATGGCACACAGTTAGTAGACCTGTGCT
GCTGTCTGTGTCTGGAGCTAGAACCAACACTGGTATCACCTCTTGCAACTCTGTGTTTAGCCACCCTGTCTCCATTCTCCTTTGTGGGCACTTC
TAGCTCGAACACTCAACAAGTACACCTGTTCTAGTGTTCTTCCATTCCAATGTTCAGACTCCTTTCTCCCCACGCAGGGGTAAGCCTGTGCTGA
CACTGTGAATCTATTACCTTGAACAGTTACAGCTTCATGAGAGGACTGCCCTGTGCCACTGTGATATCTGAGAGTTGGGGTGTCGCTGGCTCGG
GTGACAATTCATACTGAATGTCCTATCTCTTCTCCCCTCTTGCACGACAGCTCTGTCTGTGGTGATGACGATCAGTCCCTTTTTGATTCAGCAT
ACGGAGCGGCAGCCCATCTCCCCAAGGCAGATATGACTGCTTCGGGGAGTCCTGACTACGGGCAGCCCCACAAAATCAACCCCCTGCCACCGCA
GCAGGAGTGGATCAACCAGCCAGTGAGAGTCAATGTCAAGCGGGAGTATGACCACATGAATGGATCCAGGTGAGCCTGCCAGAGTTCGCCACTC
CTCATGAAGCAGGGAAGCCTGGGCGCAATTCCTCCTAGGACACACCCTGTCAAGCCGATCTGCACAAGCAGCAGATGGGTCAGGCCCCAAGGCC
CTACTGCAGTACTCAGTGCGTTTGCTGTCAAGCTTAGGCCAGGTCCAGTCCTGCAAGGAAAACGCATAGCAGTAAGATGCTCTTCCTGTGCTCT
TGAGGATGTTTTAAGGTCCTCTGGGTGGCACTAACCAGAGCAAAGGTGACCCAAAAATTCTTCAAGTAAAAGTGGAAAAGTGGTGAGAAAGACA
GACTGTGTGCCAGGAAGCCAGCCTCACACATGGGAAGTGTGGGGGTGGACTAAGGCGGGGTCATTAGGAAGAACCAGGTACAGCCTTGTTTATG
GAACTGGGAAGGGGAACTCAGGGATAAAGAGCACACGAACCAACCCATTGTCAGGCAGCCCAGCGACAGGCACTACTCTCAACATTTTGGCTAA
ATTATCTAAACCCATCTGAATACCAGCTTTCTCTTGTCTAAGCCAGAGTCAGAACACCAAACATGTTTATCAAGTGTGTTGTAGGGAAAGGACC
CAGGACATTGTCTAGTTCCAAAGTGCTCAATGAGTTAGTGACCACAGACACACAAGAGGGAAAACTTTAGCCTAGTTTATTACCTGATAATTCC
CAGTGGATTCCCAGGTAATCCACTTAGCCTGAAGCAAACAGACATGAAAGTAAAAGCAAGTGAGTAAATACAAGACAAGACCTTGGTTGATAAG
CTAGGAATCCAGTTTATTCCACCAACAGAGGCTGACCCTCAGGACTGCCAGGACACTAAGCATCATCGGACCCATGAGACAAAGCTGATGAGGT
GACAATCATTGTCCTGCAGGGATTTGTGGACAGGAGTGCAAAAAGATAAGAAGTTACTGAAATAGCCCAGATAAGGAATCGAAGTTCTGGCTAG
GGTGACAGTAATGATGACAGAAGCCCAGGGGAGGTGCATAAAGAGCATATCAAAGGGATGGCTACAAGAGCTCGGACTTAATAAACATTGAGAG
GTGGGGACTGGATGCTGCTGAGTCTGGACCTTTGAAAGGGGATGCAGTTAGTGGCGACAGGCAGGTGGGAAGGGGTGATGCCTTGAGGAGGGGA
CGTCCTTTAGCACCCTTTGTGAGCAAGTGGTACAGTCTCTAGATTAAAATGTCTACTGGATTTAGGAAGCCAAAGCCCGTAGGATTGGGACTGG
AATTGAGGCTTAGGCATCCTCTCTGGGAGGCTTAACTGAAAGTGAGTCATGAGGGAAACTAGTCCAGAGAGGCAAGAGGAAGCAGGGAGGGGGC
TACTGTGGCTGCTGTAGGGAGTTCCTCCTTGCAGCTGTCTCAGAGCTGCCCTCTAGACCCTCAGCCCTGCCACGCTTCCATCCCATGAAGACTA
AACCAGGAAGTGGGACCTTGGCCATCCCTGCAGGGTTCTAGGGTTAGGGGAGCTCAGAGAACCTGAGCCTTGAGCCCTCGAATGCAAAATGATG
CTTATACCATGATTCCGGTCAGTCCATTCTCAGCAGAGTGTAGTTTATCAAACACTTGTATCTAGTCTTGTATCACTGCTCCCTACTAATCTAG
AAGGCCAATATTCTCATCCTGTTACCGTATAGAGGAAAGTCGAGAGAGGCTAAGCTGGCGCCAGCCAAAGCCTTTCCCATTGCTTGGGCATCTT
GCTGCCTCTCCCTCTCTACCTGTGCCTCATGGTGCTGTACCACGATCCCATTCTGCAAGCACCCTCTGGTGATGACGCCAGTTCAGAAAGGACT
CCCAAGCTGCCTGCACCATCACAAGGTGTTCCTGATCCCTACTGAAAAATCTAGATGCTAATATTGGAGAGTCTATAGTCTTGATGTACCCTGT
ATCTAGTTATCATTGCCTGCCAAGCCCACAGTGTCTCTCCCAGCTGCCACAGAGACATCTGTGCTCCACTCTTCCCCTTCCCTGCCTGAGACCT
ATTCTCTCAACAGGCAACAGTGAAAGTCATCCATCCTTTCACAACAGTAACCTTCTTGCTCAAAAACCTTTAGCATCTCATGTCAGAATCTAAA
GTCTTAGCTAAGTCCCAGATTCTCCCTGGCTCCTGCCTTCCTCTATGCCATCACACAAGACTCTAGCTGCTCCTGGAAACCATAGCACACCACT
ACCTTGGTTTATCTTCCCCAAGTCCCTTCTTTCTAAAACATTTCTTCTCCATTTATTCATACAACTCTCTCGGGTTTTTGCCCATATACCACCT
CTTCAGAATCCCTGACACTCCCCGAATCTGAAAGAGCACCCTATCGCTCGCTCTCCCTGGTGGGTTATTCAAGTTGTCTTCTCCCTCACTAGAA
TATGGGCAGGGCTTTTGCTCACTGGGCAGGGTTCATGTGTTTCTTCAGGAAAAAGCTAAATGGGGGCACAGCAGAGCAGGCCAAAGTCAAGTTG
CTTCTTCAAGGACATGCCATGTAGGTATGAGTTGACTCTCCCTTACCTGAAACATTTGACATGTGTTTCAGGCTCTTTCAGATTTTAGCATGTT
TGTTTGCACACAGTGTAACAGATATCTCAGTATGATAATGGCTGTTTTGTATGCACGTGTGCTCAGTGCCCAACAGTAAGACTATAGCCTGGTG
CATTTGCATTGGGACTGTGGGCTCTCTTGTGCATCAGGTATGGAATTTTCCTTCTGTGGCCTCATGCTAGTACCCAAACCATATTTCAGATGGA
GAATCCGAGGAACAGATGTCCATATGGGTGATCCCCAAGGTCACAATGCCCAGGCACGACAGCCCAAGCTCTGCTGAGTTCACCCTGTACTTTT
GAATCATGGGTTCCCTTCTAACATATCTGTATCACTGAGCTACCTGTTCCCATCCCTGCCACCTGGGTACTGCTGGGCCCTCCAGGATGCTGTT
TAAGAGCTAAGAGAATCCTTCAAGGAAAAAGACTCTCGATCCATTTCCAGGTCATGCCTAGCATTAGTCACCCAGACACTAGCTGGAAGAAGCC
CGTGTAAGTATTCCCATAAGCTGGCTGAATATTTACCTGGCTTTGGGTGTACAGTCTCACCTACTGTGCTGAAGTAAAGATCTACTGGAGTCTA
GAGCCACCCACGTAGGCATAGCCATGGTTTTCACCTAATCAATACCCTCAAAGCACGCACAATAAGAGCCAAGGATCAAAGACTTTCAGAAAGA
TGCTTTTATTTGGGATGAAACCATACCTAGCATTAACTTAGCATTATTTAACTATGTGAGAGAAATAACATGTAAAAACACCAGGCATGGTGGC
ATGCTCTGGTGATCCCAGCTCCTGAAGAGGCACACAGCTGGATCTCCTAGGCTCACTGGACAGACAGCCTGCTAGGCAAGCTGCAGATCTTATG
CCACAAAAAGGTAAATGGTTTCTGAGAAACAAAACTCAAAGTTGTCCTTTGGTCTCCAGAGGTATGTACACCAGCATAGAACTGCATCCATATA
TTATACACAAAATACAGAGACAGGCATAGAGACAGACAGGCAGACAGACAGACAGACAGACAGATTTAGAGAGACAGAGAAAAPJAACAGGGGC
AGAGACAGAGAAGCGGGTAAAGAGACAAACAGCCAGAAGCAGAGGGCTAGASAGCCAGAGAGAGAGAGAGAGAAGACAAATCAAATGAATACAT
AGATGTCAGTCTGTTTAAATTACTCATCCTGAACAGTGCAGCTTTCTTATTTTCTGAGTAACACGGGACTTTGTTAGTTTTGTTAGTTGCATGA
TTTTAAGATACATATATACAATGTATTGGGATTCATAGATATATGCAGTGTATTGTGATTCTATTCGCCCATTACTCTCCCTTGCTTTCACTCC
AAGACCCTTCTTTTACCAGCTCATCCCCCAGGCAGGAAACAACACTCCCCTCTGCAGCAACTAACAGTTAATATCTCCTTCAGATGGGGCAGTG
TCTCATGAGCACTTCCTCTATCAGTGAACCAAGTTGACGGGTTCAGTCTTCCACCAGTATCCATTGCTTCTGTGTCTCTGTGGTTGGAATGGCC
ATGTTTTATCTGTGGGTCAGCATTTTATGACACCCCTCCCCACTCCCAGCTCTTACATCACTTCATCTCTTCTTCTGCAGGGTTCTATAGGCTT
AGGGTGTATATAATATAGATTTCCCAGTTAGGGCTGAGAGCTTATCGGTCACTTACCCTCAGCATCTGATGACCTATGACTCTCGACACTATCT
GTCACTCTCTGAAAGAAGCTTTCATGTTCTAGACTGAAAGGGATACTAGCCTATGGATATAAATATAAATATTCTGGAGACTTAGCAGCATGTC
CATTTAGGGAAACAACAGCATTATGTTCTCATTGGGTCCAAGACTCCACCAGCCACAGGCTTTTTACCACGTTTATGGTACCAGATGTGTATCT
CCTCTCATGCAGTAGGCTTCCAATCACAGAGTGGCTGGCTACCCACCATATGGTCAAGCCACCGTTGTACCAGTGGATTTATAATGCCTGTCAA
GTTGTATTGTGCAGCAAGCATGAGCACTGGTACACTTCCTCCTCTAGGAGCCTGCATAGAGCCTCTGGCACCACAAATGCCAGCCAGCAGGGAC
ACGATTCCCAACTCAATCCCAACTATTTCTTTGTGTCTTGTGACCAAAATGTGTGTCATCTTCATCAATAGCCTCATACCTTCTATATCTGGTG
GGTATCAAGAGTTGCATAGCATTTGATAATTTTCAGAAGTCCAGTTATCATAACACAAAATGTAGAGGAGGGCCAATGGAGACGGTTCAAGCAG
AAGGTGCCTGACAGACACCATGCCTGACACCATGCCTGACGACCTCAGTTCTACCCCCACAGAGCCTACATGAAAGAATGAAAGAACCAACTTC
TTCTAGTTGTCACCTGATTTCAATGCCCATGCCATGGCATGTACCCACGCATGTAGTTTGCTCAAGCACAGATGCACACAATACTATTTAAAAT
GTAGAGGAGGGTGTGTCAATGCTCCAAAGAAGAAAACAGAGCAGGGCAGACAACTTGGAAGTTCTTGGCTTATGCACCAGGTCTGTGGCACCCA
GCACTCTGATGCAGTGGCCACTAACTAGCCCCATGTTGCTGAGCACTGGGACTGGAGCTACTCTATGAGTTAGATGTGCTATAATGTAATATGC
ACATTGGACCTAAAAGTCTTAGTATGATAAAATGTAAGATATCTTGGTGACTGTTGATTCTGCTGAAATAATAATCTTGTGGCTGTATTAAAAT
ATTAATGCATTTATCATATTATCATATATAATATTTAATATATTTAATATGTATTTCTTATCAAAATATATTTAATATAATAATCAATAACATA
ATATATTGATATGTTATGTTAATGAATTTATTCAAATTCATTTCATCTTCTTAGATTTATTTATTTACTTATTTATTTATTGTGCGCCGGACCA
GTTCGGTGTGCAAATGTGTATGCGCTTCACGTGCATGCCTGATGCCTATAGAGGCCAGAAAAAGGCATCAGATAGATCCCCTGGAATGGAAGTT
ATGCATGGTTGAGAGTCACCATATGCCCCTCTGCAAGAACAAGAAGTCCATTTAACTGCTGGGTAATAATATCTCTGQCTTCTCACTTTTTCAA
TTTPAAAAATACACCTACTAGTAAGGTGAAAGTGACCCTTGTGTCTCAAATATGTAACTCACAATCTACTTGGAGTGGACCGTGATGTAGCAGA
ACATTTCCGATTGCTCAGTGATTCACACCACATCCCTACAAAGTTCTTTCAGAAGCATGGGTCTCTGGGTTCCTTCTTCACGCTTATTTACACT
CCATAGATGTCCGTGTCTCTCGCACGAAGGAAGTACTCCTTGTCTCTGAAGCTGCAATACGCTCTTCCCTTTTACCCTAGCAGCAAGCCCTCTA
GACCAGGGTCAAAGGGTCATTCAGAGGGGCTGAGATCTGAGGCAAGAGAGATTGTCTCCGTTGCCTGGGTCGTAGGAGGTTTACTCTGTCTCCC
TTCCACTCCTGTTGTGGTTGAATCTGGTGCTCTGGGTTGCCCTCCAACAGCACAGCCCATACGTTCTCTGTTGGATATAAGACTTCAGAAACTT
GATCCAAGCTGTGGTTTGCAAATAACTCTCAGCTGCATCCGGTCTCGACCATGGGAATTGCAGAAAACAGTGCATGACGGAAACACTAACAGTT
TGTGAGGGAGAAAACAGAACTAGTTTTAATACCTGGCATTCAGGAGCTAAACTCAGCATCACTGCCCTGACACAGGCTGGACTGGCCAGCGTTA
TCAGGAGGCATCGAACATGCAGAGAACCCTGTTCCACTCTCAGGAAGATGACCCTCCCAGGTACCTTCCTAAATTTCTAATTCTAAACCTTGCA
TGGGGAGTAAGACACATATCATCTGTGTTGAAAGCCCAGGTAGTCCGGTTTTTGTATGGCTTTTGTTTTGTTTTATTTCTTGAAATTATACTTC
TATATCAAAACCAACATGCCACTTCTACACATGGAAGTTTCTCACAGAAACCCTGAAGGCCGCCTAGATTCCTCAGGTGTGCAGTGTGCATGCT
ACATGAATGACACACAGACAGATTCTGTGACCGTGAPGCTCAGTGGTCCCTTGCTGGCTGCGTGGGCATCGGGTGGTCCTTGGCTCTCAACAGA
CCTGTGATCTCTCCAGGCACTGTGATCAAAACGGGAAGGTAACTTTGGGAGAAGTTTGAGATCCCAGTTAACCCCTCTCAGGGTTTCACATCTC
CCTTTATATTGCCTTGGAGTCTGAAGACACTTCTATGGACCCCTAGCAGTCTTAAAGCAGTGCTCAATAAACACACAGAATAGATGCTTATGTC
TAAGACGGTTACGCTGTACTGCTGAGGCCTGCTGCCTGTAGCCTCCCGGGGGCCTGAGGTGGAGATGCTCTGAGTTATACCCATCACTTCCTCC
TGCTCTATTGTGGCAGCCTTAGGCCCTCCTGTCACCTGCCCCAGTACAGTCAGCTGTCTGTGGGCCCTGACCTCTTGCTCTCTGTAAGTGCGGG
TCTCAATACAAATTAGCTGCAGCCGTATGGGCAAAATGAATGGCCAAACCTCTTACCCCGCCAGGGCTCCCGCCTCCCCTCCCTCTTGTTCTCC
TTAAGCCCTCTTCACAGGCACCAGCTGCCTCATTAAAGAGCAGCTTTTATGCTGGGCTTCACCTGGGTAGGCAGTGGTCACCAAGCCCATGGCT
GGGCTGGCTCGCTGAGGCAAAGGATAGAGACAAACCTGGCAGGCAAGTGTGCAAGCCAGTGAGACATGGCTTTCTCCTTTATCACCCTGAGCTA
GACTAGCAGGGTGAGCTCTGTAAGGGTAAAGGATAAGGGTATCAGGTCCTTGTCCTAAGGAGGTCCCTTTGAAGACCTTGATGGAAACTGTTTG
GGACCAAGAAGGGGCCTCATTCCAAAGAAGGCCAGGAGGGACGCTCAGGCAGGTAATCAACCACCAGGGGTCATACAGCTGAGGGGATAGATGG
AGGAGCCTAGGACTTCTGTGGGCTAGTGATGCTACTTACTGGTCAGCCAGGCACTCCCAGCGATGGAGCCCAGGGGGCCCCAGCTCCTCTGGAC
CTGCTTCTTGGCCATGCCTAGGGCACCCTTGTTGCCATTCATCCCCACCAAGCACGCTTGGTTTTATGTCTCATCTTTTCTTTGTTATCAGTTT
TTTTTTTTTACTCCACCGAGTGGAAGCAACCTTTCCACCCATGGCCACCGTACACAGCTCCAAGTCAGGGAGATCCACTAGTTTGGGTGCTTCT
GAGGTTCTAATCTCTGCCTGGAAACAGGCCAATTTTAAATGCAGAAATTCCAAGGCCAGACACCTATGAAAAAGAACATTTAAAAAACCCTTCT
CTCCAGTATCCGTAAATGGGAGACTTTGAAGTAGGCACCTCCTCTTTTGCTACCATTGATCCTGGCTGGCTGGCTCTGGGCACCCTGCATGGAA
AGTGTGTGTGCTGTCCACTCTCCCCACCCCCACCAGCCAATGAAAATTCCAGTATCTTGGTTTCAGGCCAACTTAATTCTCCAAGCTTCTCTGG
TATTTGGATGGTTTCTCATCTGACAAGAATCTGGGGAAGACAACCAGTGTGTGTGTGTGAGTGTGTGTGTGTGTGAGTGTGTGTGTGTGTGAGT
GTGTGTGTGAGTGTGTGTGTGTGAGTGTGTGGGGGTTTTACACACACTAGCATATAGGTGCACATCTGTGCTGGAGGGTGTGTATTTCTCTAAC
TATATCTGTGTGATAGCCTATGGATGTGTGCATGCATCCCTGCATACA1TATATGCATACATTATATACATGTAACTGCTAGAGTATATATGCA
TGTGTGTTTTATGGGATGATTGTATGTATGTACATATTGTATGTAGAACTATGTGAATGTGTGTGTGGAACATGTGAACATATTTGTATTGTGT
GTGTAGACCTATGTATATAAGTGTGTGTGTGTGTGTGTGCGCATGTACTGTGTGAAGTAGTGTGTGTGTGTGTTTGTGTGTGCATGCACACATG
TGCATATCTGTGAATATGTCTTTTGATGGCCGAGTACATACATATATGTGTACATGTTATCACTGTGTATATGATGCCTGTGGTAGAGTGTGTA
GAGCATAGTGAATTCTGTGTGTGTGTATGTGCATATGTACTAAAGTTAGAAGCTGAATGTTTTCATTATGTATGTGTGCTGTGTGTAGTGAAAT
GTGTGCGTGTGAATGTGTATGCGTGCGCACACAGTTTCCTGGGGCACAGCAGTATGCTCTCGCCAGCTCAGCTTAGACACAGGCACCAGGGTCT
GACTGGGGCTGCTTCATCTCCAGCCAAGTCCTTCAACAGCAGAAGCCAAGGGCTGAGCGCTGGTGAAAAAGCCCCGATGTGCTACTCCCTCTCC
TCCGACTGCCATGCGGACATTAAACCTTCAACAATCTGGGACTCTGACGGCTGGGTCTTGGTTCTTTGCCTGGAAAACAGTACGCAAAATAGTA
AAACCTTAAATTCTAACATGTGTAAGTTATACATCTTATTTTCATTTTCAGCCCATGTTCTCCTCGGTAAGACATCAGGAAAAAATTACCGTCT
GACTTCTAATATCTCAGTGGAGCATTTATCTCCCAGCCATGATAAGTGATCTGCAATATTTTTTTCCTTTCAGATTAAGAAACAAATTCATTTA
ATTTTCTTTATTGAATGTGTTCAGGCCCTCCCTGCTCTGCCTGACTCTCAGGAGCCCTCTACTCTTCTCCTTCTTCCTGGGCTGGTGACTGTCT
CCTTGCTGTGGGATGGGTGACTCCCTAGTAGGCAAGGCCTGTTCCTTAAGGGCTCTCAATCTGTCCCTGCCCACTGCAGAATGACGGACAGCAG
ACTGACCGAGGGCAGCAGCCAGGCGAGGCACGTAGGGAGATGTTAGCGTGCAGAGCTGGCAGACTGTAATCAAAACCTCGTCTCATCCCCTTGG
CCTGAAGGCCGGTTTCQCACTATCAGGAGAACTCCCCACTTCCCAGGCTGTTACCATGATGCTTGCTGTCTGGCACTACCATAGTCCACTATCC
ANNNNNNNNNNNNNNNNNNNNNNNNNNNNNNNNNNNNNNNNNNNNNNNNNNNNNNNNNNNNNNNNNNNNNNNNNNNNTGCTCAGTGCCAGATGC
TGTGAATATCCACTGCTAACTGTGCTTAGTCTTAGTCCACCATGGGTAATGTGCCTTCTTCCTCTTGCACGTCCATATTGCTTCCTATGTGCAC
TTCCATATGTGCACTTCCTCTTGCACTTCCATAGTGCTTCCTATGTGTGTTTACTACTCAGAGCTGTAAAAATGCTACAAGAGTCATACATAAC
AGGTCACAATGCATTCCAGGGGTTACTCTTCTTTGCAGCACCCAGCTCCTCTGTATGTGGCCTTTAGTTGAGAAGTTTAACACTGGGACCTTGG
GTCCTGCATCCATCTTTACTAGCCATGTQAAACCAGTAAGCACACCTTTCCTTTGGTAACCACAAAACGGACGTAGTAACCTTTGCTCCACACA
TCTGAAAGGGTTATCAGGAAACTCAGATAAAGCAAACAGCTTATGAGATGTATTAAAATCCTACACATGCAGTAGAAATAGCTGAGGCTAGCAA
GTACTGATACCATCTCAAGAAACAGAAGAGACAGGGTAATAGGCTGCACAGAGCATCTCATGCTGGACTCACTCAGTCCTGTAACCAAGGTAGA
CATCAGCGTCTATAGGTTTCTGGAGAAGGCCTATCTATTACAAGCACTAGTGAAGGTGGGTAAGTGTTGACCACTGGCMJCAGGTTGAGAACTG
GACACACCAGACAGTGGAGCAGGGCTGCAGGCCCCTGAAACAGGAACACCTAGGACACACAGGGTATTTATGACAGGATCAGGATAGGTTCCAC
GAGGAAGGTGGCACTAACAGTTCATTGTCATTGTTGTCATCATCCCAGGGATGTCTAAGTGACTGGCCAATTCTGTTCAGGCTGGAGTGACATG
TCCCCATAATGATGGCTTCTTTTGACCTGTTTCCTCTCACTCCAGGGAGTCTCCGGTGGACTGCAGTGTCAGCAAATGTAACAAGCTGGTGGGC
GGAGGCGAAGCCAACCCCATGAACTATAATAGCTACATGGATGAGAAGAACGGCCCCCCTCCTCCCAACATGACCACCAACGAACGGAGAGTCA
TTGTGCCTGCAGGTACCTTCGGAGTCAGCTGCCTGGCCAACGCCCACACTTCCATTATTCAGGGGCTGTAATGGAAGGTTGGCCCATCTCTTAG
AACATGAAAACCCAGCACCCTAGTCTTTAGTGGCAGACTCAAACCAGGGAAAGTTGCACATCATACGTAGCTACCCTCCAAGGCAGCATTTCAA
ATGTCACCCGAGCCCTCGCATGCTGATTCCAGAAGCCCCACTGTCAAACATAACACAAGGCAGATTTTCAGTGAGCAGCCTGGTACCCCCAGGA
TGAATCTTGGGCAGAGGCAAGGCTGCTCACCCCGCCATTCCTTCTTGTGAGAAGTGCAGCTCCCGATGTCCACCCTTCCCTCAGGCATGTACTT
CCTGCCTTCTGCTAGATTCCACCTTCCCAATTACAGCCAACTCTTTCAGTTTTAGTTAATAGAGATAACTATATTTAACAAACAGAACGCCTCC
ACGTGTGTGCCACAAGTTATGTCATTATGTAGAAATAGCCTTTTTTCCTAGGTATTGGTGCTGTTGCTTACGCAGTGGGGGGCCGGGGGGATCT
ATTGTTAAATCCTCTAGCCTATTGGCTGCATGCTGCCTGGGCCAGGCCCAGTATGCTCAGCTGCCTAGGTATCGAGGGAATTCAAAAACTGCCA
CTTTAGAGTGGGTGCCAGGTGGAAGGGGCAGAATGTCAGGAGTTTGAATAAAGTGAATTACAGGGCGGGAGCTAAAACTAACCTTAACCAAAAT
AACTTCACCAGAGAGCCTCCATCCTTCCATTTGTAAATGTCTAAATGCAGTTGTGTCTTAGAACGATAGTTTGTCTTGTTGATCTCAAGGATGA
TTTTTGTGATGAGCAAGTCTGTCCTGTCCCTAGTGTTTGTTTCCATGGGAAGTTGTATCTGCTATCCTAGTGTGACAGTCACATTTCTGACCTC
TGGGCTAAATTCCACATGGGAAAGAGTGCAGAAGGTATCAGAAATGAAGGCAGAAGAGGAAGGGCTCTTGCACCTCACAGGACTAAAATCTGCC
TGGGCTCAATCTTCTTCAAGCATAAGGCGCTGATCGCAAGGAAGGCGTCACCTGGTTTATTTGTTTGTTTGCTTGTTTGGGCTGTTTGAGAAGG
GGCTTCACTGTGTAGCACATGCCGTCCTAGAATTCACTGTGTAAGCCAGGCAGCACCTTGCGGCAATCCTCCTGCTTCTGCCCCCTGAGTTTGG
GAACTAACTATAGGTGTGCACCACCCACTCCTGGCCAGAAGGTCTTTATTCAGCCAGATTTCACAGGAGCCAAGTGCTTTCTCTTCTGTCACAT
CATCTAATTGGTTTGTCACCACCCTCAGGAGAGCAGCTGGTGGPGGTTAGCTCACGTGCGTCTTCTCTATTTTGAGGCCGTGATTCTTTTTAAG
CAGACGTGGTGAGAGACTCTCTCCACCCCACTAGGCCAGGAGAGGCCTTTGCTATCCAAATCGCCTGCTCTAGCCTAAGAAGGAACTCTTAGGG
GATCAACCATTAGAGGAATGGAGATTACCTCAGGACCAGGTGATTGTCACCACCTTGCTTTGGTCGTCATAAGGCAGACTGTTCGGTTGTCTCT
CTACCCTTCACGTAGTTAAAATGTTCTTACGGGGATTGGCAAAAGGTGGCTCTTCCATGAACCGACTTAATCATAGCAGTAAAAACAAAAACTG
GCATGTTPATATCATGCTGTTTCAAAGCATTTGGCATAACGAAGTCATGAGCTTTAGATCCGCTTGGTTATTCTCCTACTGGTTAGATCTGTAT
GCATTACTCCTAGACGCTCTGGCTTCCTCCAGCTCAGCCTCCTGGACGCACGGCAAAGTCTCACTTGGGCAAGTCCACAAATCAAGCCAATAAA
ACATTAGTGGAACAACCGATTGAATTTATGACGGGATTTTAAGACAAGTATAAATGAGGGTTCTAGTTAATCACCATTTCCACATCGAGATGGG
AAGGAACAAGGGCTAAATCGTCCAGCAAATACATTTGGAAGAGTTTACCATGATCCAGAGTGGAAGATTTATAATTCCTTTTACTGGAGAGGGA
GAGAATTAACATTGTGACAGAGCCCAAAGCGAAGAGCGGAAATAATAAAATAAATAAAATATAGGGCATAAAATTGAATGTACAGTCTAAGACT
CTAGTAAGTTCCTACAAGTAAGGAACTAGGGGAAGCTATGGATGAAATGATACACCACATAATCCAAGCCCCTGTGGGACATCTCCTTTTGAAT
TCTCAGATTTCCATTCATCTATAACCCTTGCTTGATGGTTTCTGAAAAGTAGAGGGTTGGAAACAGAACCAGGAGTTTATCTCATAGACACAAA
CCCCAACATGACCAAGAGGGCTAGCTCGGGGAGATCTTCCGGGTCCTGGAACAAGGCCTTGCAGTTCCAAATGAGTTAAGGCGGGATCCTGACA
AGACAAAGCTGGGGCGGGGGGGTGTTCAAGGGGCCTGCTTCTCCAACTATGTGACATCTTTGAAGGCTTCTGTAAAAACCCTAGCAGTAGTCAC
GAAGGCAGAACTTCCACGGTTCCTTAGCCATCTACAAAATGGGTATAACAAGACCCATTTCATAGATTCTCATGAGAGAAAAAAATGGGTCAGA
TAGAGAGCTGGCTTGTGGTAGGATCTCACAGATGGACTATTACTCAGTAGGTACATCTTGGAGTCTTAGTATGACATACAGAAGCTGTGACATA
TGACAGATTTTTTTCTGTGCTCCCTCTCAACCTCTCAAGGGTTTCTTTTATAACCCCTAGACATCACAGCACCTCACTCCCTCCCCACCACCAT
TGCTGGCCATTTAGAACTGAGTTCTCTTAGCCTAGATCTTTTGCAGCCTCTAGAATGAGAGGAATATGAGTTGGTTTAAGAGGAGTACACGTTT
TTCCTGCTAAATAAGCATGCTAAATGAAACAATCAAGCAGAAAAGCAGAAGCAGGAGCAACCAGACACCCTGTAATATAAAATACCATCCCTTC
TCCAGGAGCCTTCCAGGACTCTCCAAATTCACCCTAGACCCTTCAACCCAAGGTTCATTGAACTAGCCAGCACAACCCATTGCTTAGCTATTGC
TCTGATGGCTGCATATTTATGCAGATGGGTGTGTGTCTGTGTGTGTGTGTGTGTCTGTGTGTGTGTGTGTGAGGGAGAGAGAGACAGAGAGAGA
GAGACAGAGAGAGAGAGACACAGAGAGAGAGAGAGACAGTCTCAAATACTCAACCCTCCTGCCTCCGCTTCCCGAGTCCTGGGATTGCTTGAAT
GTATCAGCATGCCTATGGAAGTCTGTCATTTATAGCATCTCCCACCTTAGACTGAGCAGGTTQAGAAGATGCTTCAGGGAATSATGAAACAGAG
GGGGCTGGGAGAAGGCACGTTTGGAGCAGAGGCCTCACTCCTGTCCTGTTTCCGCTCATTCACAGTTAAAGAGGTTTCTGTCTCTAGAGCCATT
TCTCATCTTTAAGTATTTAAATATCCTAAAAATGTTTAGGGGTGGTCTGGTAGAAATCTTAACACAGCTGTCCCCCCCCCCAAAAGGAGGAAAC
CCAGATTGGATTTATGTCAAACAGTAACGCCATTGGGTTTTCCAGCTGCCAGGACAGAATCACAGGAAAAGCAGGCTTGTGTATATTTCCCTTC
CGGAGAGTCCGGACGGATAAGTGTGCCCTCCTGAATTCACTGCTAGTCTTAAGATGCAAATGGGTCCCCTTCCCAGAAACGTTAGTTACTGGGG
ATTATTTTCATTTTGATGTCACCATTTCTTGGGCCAAGCTATCACTTGACCTCCAGAAACTTTAAACACCATCCTGTCAGGGTATGTGTTGAGG
CAGCTAAGACCTTTCTCAGCCCTAAGTGGTTCAGGCAGTGTCAGAJAZCCATGGAACCAGACGGAGACTTCTGAGGGGACGATCTGGAAGGGAA
GAGAGAGGAGGTAGAAAGAAAAGAGAAGAGGGAAGAGAGGGGCATAGGAAGGAAAGATGGGTGGGGGTGGGGGGGTTAAAGATACCATGAATCA
GGAGTGAGAGAAAAAGAAGAGGAAAAAGGAGAAGACCGAAGAAAGAGGAGAGAGCAAAAAAAGAGGAGAAGAAAAGAGGGGGCTGTGGGGAAAG
AGGACAGAGGTGGAAGAAGCAAAGAGGAGAAGATAGAGAACAGAGGGTGGAGGGGAGCCTAGGCTGCTGGGCAGGCATGCATGCTGACCGTTGT
TATCTCTCCACTCCAGACCCCACACTGTGGACACAGGAGCACGTTCGACAGTGGCTGGAGTGGGCTATAAAGGAATACGGATTGATCGACATTG
ACACTTCCTTCTTCCAGAACATGGATGGCAAGGAATTGTGTAAAATGAACAAGGAGGACTTCCTCCGAGCCACCTCCGCCTACAACACAGAAGT
GCTGTTGTCGCACCTCAGTTACCTCAGGGAAAGTAAGTACCATGTACACCCCAGGGCTTGGGAGCAGGTCCTGCCAGGTCCCCAAGAGCCAGCC
CAAGGGAAGGATACCCTAATACCAGTCATGGAAGACCTATGCCAAGGAACAGTTTCCTTAGGACAGGAACCCCATCAAGGTCACATGTGTTCCT
ACAAAGCCTCCAAGGGAAAGATTTCATCCAGGAGTGGAGTGTCCAAAGGAAACAGATAGAGTATGGAAAACTCACAGAGACCTCGGACATAGGG
AGGATGACACCAGCTACTAGGTAGGCTCTGGGAAGATCTCTCTCTTCAGAAGCTTCTGCTAACCAAAATTGTACAGAGATAGAATCTCTGGTTG
GAGGCTTTCTAAGGAAATGTTAAGAGPTCTTACCGQAAGTTCTCAACATAGATATGAGATCTAAGATTGTGGGAGCTGACAGACTACTCCTCCT
ATTCTGGATTCACATTGATGCCCAACCCTAACCTATGGGGCTGCTTCTGCCCACTGCCACGAAACAACGACAGCATCTAGTGGCCCAGCTCTGT
TAGGAGGCACTGAAAACTTTCCCAGACAGAAACCCAGGGAAAGCTTAGTAAAGAAAGATCTACCCAATAGGAGGCTCCCTAGTACCACGCCCCG
CCCACCAGTTCCATGTGAATTTTGATAGATATGAAAGGGAATACCTATGGACCTGCTAGTGACTCAGCCTTAACTCTCCATCAGAAATCCAGAC
TATCCCCAAGCCTGGAAGTGAGCTCAQTGGTCTGCAAAGATGAACTGAAGCCACAGGAAGGCAGGCATGGGCTCATTCTGCATGAGAGCATCCC
TACAGCCAGCCACGTCAATGATTAGCGGGGCAGAACAAGATGCACTGTCCTCTATGAACGGCCTAGTTTGCCAGAGGTATCGTAAGAGTTTGAG
AAGATTTGATTGTTTTAAGGCACCTTTGAATTCTCAGACCTCGGCCAACACTGTGGGGCTGCCTTTTGTGGGTTCACGGGCTCTGGAATGGTTC
ACTTTGTATAGGATCTCCATTGTGCTTTCAGTTGTTATCAGTAAAATACATCGTCTCAGTGCAAGGGAGTTCTGTAGAGAGCAGGGCAACACTC
TACTCACATGGGCTAGAAAATGAAAAGTTAAAATATTTAAAAATGCATTTGGAAGTTTCCATGGGTGCCTCTGGGCTGGCTATCTTGCCACCTC
TCTAAGGCATCTGTTCGTGCTGGCAGAGGACACATTAGCTCCACTGCAATGACCTCATGTCTGAGTTGCTGCACCATGAGGTCAGTCCAAGAAA
TAACACTCTAAGAAGGGGCCATTTTCTACTTAGTCTCTCCTGACTTTCAGATGCTTAGCATTTAATAATGATTTGTATTTCTCAGCCTTTAATT
GCTTGGTCGAAGGAAAGAACAAAAGGATCTTTTAACGGAGGTCTGCCATGATCTGCACAAGCAGGAGAGGCAGTAGGCACAGAGCATCAGTGTA
GAAAGCAGAGGCCAGGACCATAGCGCCATCAAGCCTGACTTCTAGGCTCCTCCCGCATCATCTATGCAAGAGGTTGAGTGATCCCTAAGGAAGG
AGAAATGTCAGAAGCTCCAGTGAGCTCTACACAAAGGGTCCAATAATCTTGGCTTTCACAGTAAATGCTTTGTGAGGACCATGTCCAAGAACGT
TTGGACTTCATAAGAGAAGCCCATTAAGACCATGTGACTTTCAGGCCTGGATTTCTGACCAACCCGTAAAGACAGCGGGAGTTTCTATTTCTTC
CTTCTGTCACCAAAGAATCTGAGCCACAGATACTGACATGAGGCATATATCTGCTGCTCAGAAGGTCTAGGAATACTGTTAATCAGTCACTGCT
TCCTATCAAGCCTTGGCAAGCCTGCTAGTAGACAAGGGCAGATAACAAGGCATGATGAAGGTATGAGCACCTATAGAGGGAGCAGGGAATAGAG
TTTGGTGTTTGGAGGCTAGTGATAAAAACCATGTAGTCTCTCCTTTTAAGAACACATGTAATCACTATGAAAAATGTCATACAACACACGAAGT
TTCCAGAAGGGGGATTTGCTATGAAGGAATTTCCAGAGTTGAGAAGAGGTAGTACATGTGTATGTTTGGAAAGCCTTTGAAAGGGCAGTAAGCA
TGTGGTGATAACAGTTGGGAGTTCTACACAGGGAGAGTTTTCCTGCCACCAAAATGGAGGTAAAACATAAGTGTTGCATGTACTTTTATGCTCT
ACCAGCCTTTGCTCCAGGAAGTGACCATCCTACCAGCCCCCTCCCAGCTTCCCATGAATCTGCAGATAAGAGACACAGACACTGGGCTGGAGAG
ATGGCTCAATGATTAAGAGCAACTACTGCTCTTCCAGAGGTCCTGAGTTCATTCCCAGGAACCACATGGTGCCTCACAACCATCTGTAAGTGGG
ATCTGAGTCCCTCTTCTGATGTGTCTGAAGACAGCAACAGTGTACTCATATGCATGGACGGAAGGAAGGAAGGAAGGAAGGAAGGAAGGAAAGG
AAAGGAAAGGAAAGGAAAGGAAAGGAAAGGAAAGGAAAGGAAAGGAAAGGAAAGGAAAGGAAAGGAAAGGAAAGGAAAGGAAAGGAAAGGAAAG
GAAAGGAAAGGAAAGGAAAGGAAAGGAAAGGAAAGGAAAGGAAAGGAAAGGAAAGGAAAGGAAAGGAAAGGAAGAAAGAAAGAAAGAAAGAAAG
AAAGAAAGAAAGAAAGAAAGAAAGAAAGAAGTAAGTGTTAAAAAACAAAAGACACAGACACACAGTTGTTCAATTTCAACTTGCCTTCTTGACA
TAATTCACTGGGGATTACTATCTCCTGCCPGCAAAATGCACACTTCTCTATACTCTTAGCTCTGTACCTCCCACCTGCCCTGACTTCAGTGGCT
AGTTCCATCTAGCCCCTCCCAAACATCCCTGACCAACCACCTGTAGTTGTGGCCACAGGCCCCAGCTTCTCCCAAGACCTCATATGGCTGCTCC
ATTCTTCTCCCTCC2AGGCTTGGCAAAAACTCCCCCTCTCCTTCCTTTCCTCTCCCTTTTAATCCAGCGACCCGGAAGTCCTACCTGTACCTTC
TGTCTAGCGATTGCCCCATGGCTTTCTTTACTGACAGATTAAGAACCAATTGGGGAATAGGACCTCAGTATCAGACCTACCCTTACACATAAGA
TGTGCTTATGACACAAAGTTGAGTCTGTGGGCCAAAACAAATATCATAATGGTTGCATTAAATTTAAATTTTCTTGAAATGTAATACAGATTGT
AATAACCTTCTGAGGCACCTGGATTTAAAATGATCTTCATGAAATTTTTTCTGACTTTTTTTTTCAAATTATGGTTTCTTTTTTAGGATTCTAA
CCTCTAAGACCCTGGTTATTGGGTACTACTTGGCTCTATCAACCCTAGAGGAGGCCATTTGTCAGACATTGTCTGCTCTGTGATGGCTGAAAAC
TTTTGCCTGCTAACACACATGCCTTCAAGCCTTCTCTGTTTTTGTTTTTCCAGACTGAATTACATGAGCTATTTCATCATAGAGAGAGAGACCA
GGGTTTGGCTGCAAGGTTGGGGCCAGAGGAACAAGTGTAGATTCTTAGCATCATTGTGTTGGGCTTTGTTGGGGAGCAGCTGTGGGCTAAATGA
TTCCCCTTTTGGCAAGACATAGAGGGAAAAGAGCTGAGACTCACAACATAGATGTAGGGCACAGCGTAGAGTAAAGGGAGATCAATACAGAAAA
ACTGGGGACTAGCCAAAGCATGGATTGATGGAAGTCAAAATTGGCTCAGCAAGATGGGTCAGTGGGAGAAGATACTTCCTGCTAAGACTGGCAA
TCATAGTTCAATCCTTGGGACCCATATGATGGAAAGACAGAACCAACTCCCATTAGATGGCCTCTGACATCCACATATACACTAAAGCATACAA
GTACACACACACACACACACACACACAATGTAAATTTGATTGCACTTTTCTAATCTCTCTTATTTCTGAGTTTTGTTTTTTGTTTTGTTTGTTT
TGTTTTTGTTTTTTGTTTTTTGTTTTTTTGTTTTAGATTCCTTGTAGGGCGCTGCATTCTTGCATCAAATTTACTTTCACAAGAGACAGTTGGC
TATTCTTGTTGTCTCCGAAGAAAGGTCTACTTTAAGTTCTCAAAGATTAATCATAAGTTAAAGAGGAATGGCCAATGATAAGGAAGAAATAACA
ATGTCCTCATGGATGAGCCCTGCCTAGAGGGTCTGGCTCTTCACATCTGGGCCTTTCTTTTGGCTGTTCCAGATGCGGTACCTGTGTAGCATTA
GGAAAGAGATGAAGGGACTAGAACTGACTAGGGTACCTGCAGTTATTGGAGTTCAGCTGAAATATCTGAGACACAAGCAGACACCAGCCCAGGT
GTCATTCACTTCCTGTGGACAGAACACCCCCTTGTGGACACCATGAACAAAACTTTGATTCCCATTTCTTGACCGAAGCAACTGAGGCTGGCTA
GTTTCAGGCGAGCCCATCCAACGGTATCTCGCATGTACAGCACCTGTCCTTACCTTTAGTCCTCGGACCACTGAAGCTTTCAGTCTGAGCTAAG
GTCACTCCTGCAGAGGACTTGATGACTCTCATCCAGGATGCTTGAATATGCCTGACTTGAAGATCCTGACACAACAGACCACACCTCATTTGTC
TCCCTTCTCATCTCCAGGGCCAATCTCTTCTCCAGCCTCCAACTTGAGGGCATAACCAATGTCCCCATATGGCTTTCCATTTGTGTTGCACCAA
TAGATGCAGCCTTTGCAAAACTCCTCTGCAGCCTCCAGCAGTGAAGCCAATCAAACCAAATCCCTCCTCTCACGCACCCTGACCTCCTCGATTC
CCCCAGACTCTGGGTGTGATTGGCAGCTCTGCCAGGATCTCCCAGATATGCCCACTGGACAGAGACACTGTTGGCAGCTGAGAATGGGCAAGTG
AGATCAGTCCCTTCTCAGGCCAAGGCCATGACGCAACGCTATCTGTCTCCAACTAACAGTTCAGTCACAGAGCCACTGGGAATGAGTTTGGGGA
TGTCTGGGAGCCCAAAAGCTCCTGGGATGAGTGGCCCAAAGCAGAAGAAAAGGGGTTTTGTTGTTGCTCCTGTTCGTTTTCAAAGGAAGTGAGT
GGAATGTCCATCTGGGCCTGAGCTGGGAAACCTTCTAAGAAGGCACACTTTCAGACAGTGGCAACCCCAGAGGATGAAATACATCAAGTATCAG
GGACACCACAGAAAAGGACTCCAGCTTGATTCCCATTTTTAATTTAAACGTAATGGAAAACACATTTTACACTCTGTGCTATCCCCATCTGCAA
CCTCAGATGGCCATGCAAAGGTGATGGCCCAGTCATTAAGGCAAGGAAGTGTTAAACTTTCCATCTGGATCAGGCCAAGAAAAGAAGGCAGGCT
TCCAATAAGATAAAGTGCCCAGTTGCCTGAAACTCAGTAGTCAGCTGGCCTGTTGTTTAGCGTGTAGAGCCTTAATTCCCTCCCCAGTGTGCAT
TTAGGGCTGGGGCTCGCTGTAGCACAAAGAAAGTGAGCGACGAATTGACTGGCTGCCTGGGGTTGGGTTGGGGTAGATGCTGCTGGGTTCTATG
GTAACATGAATGGAGCCAGAGACAAGGCTCTGGCACCAGGTCCCCTGTCCACACTGCTCCAGGTGTCCAGTTTGAAAAGCAATGTTAGAGAGTC
CGCAGAGCTAAAGGAAGCTAAGGACTTCCCATCTGCAAGTCCCATCCTATGTTCACTTTACTAACTCCTTTCAGATCCCAAAACGGAAAACGAC
CTTGCTGGGCTAGGGATGTCACTCAGTTGGTAGAGCACTTGGCTAGCATGCAGGACACTCTGGTTTTGATCGTGTGCACCCTAGAAACTGGTGT
GGTGGTGCCTGCCTGTCATCTGCAGCACTTGGGTGGTGGGAACAGGAGTATCAGACATTTACGATGGTCCTGGGGCTGTGTAATGGCTGCTAAG
CCAACCTGGTATACATGAGAATCTGACTTAACAAAGAAAAAAGAAGAGGAGAGGGGGGAAAGGAAGAGGGGGAGGAAAGAAGAAGAAGAAAAAA
GAGAGCAGAGGAAAGAAAAAGAGAAAACAAAGTAATCTTGTCCTCGAGCTCTTAATCTTGAAGGACCGGGTCACTACCAACATTTTAGAGCAAA
ATGTGTCTCAAATCAACAGACACATATATGTGCACATGAATGTATCCTGTCTCACACACACACACACACACACACATAACAGAGTGAACAACAC
TTTAAATTAGGTATAACATGCAGGGAGTTATGAAAATCACAAGGTACAACTAGCCCCCAAGACACCTGCCCTCACCTTCCAGCACCTTTTCTTT
CCCTTCTTCCTTCCCTCCCCTCCCTCCCTTCTTCCCTTCTGCCTTTACTACCCCCCTTACTATTTGTTTGTTTGTTTATAATTTCTGGGACAGC
CTCATTGTGAACCTCAGCCTATTGGAACTCTCGGTGGTGCTCTTGCCTTAGTCTCTGGAGTGCTGGAATAAACGTGCATCCGTTCTTGCCCATC
TTGCCTGCATTGATTTAACCTAAAGGATGCTCCTGCTTCTGAACTGAGGACAGGTCTTATAGAGCAGCCCATCAGTTGATCTTGTCTCTTGGCT
CTGGTTCCTCTGGCTGTGTGATCGTAAGCCTTGACTTCCCTAAAGGTTGATCTCAGCTGTGGGAGGACACTAGCATACGCATCCTTTGGGTGGC
CCCTTCAAGGACTAAGTAAGAGAGATAGTGTCTGGGAAGAAGGGGGGATGGTTTAGTCAAGAAAGAGCCAGGCATGCTAGTGTGAAGACTTGAC
TTTGATCCCCAGAACCCACGTGGAAAAAGCCAGTTGTGACAGCATACACTTGTCTCAGTTGTCCCTGCACTGCGGAAGTGAAGACAGGTGGATT
CCTGAAGCTTACTGCACAGCCAGCCTTACCTATCAGCTGGCTCAGGTCATAGCAATAGACTCTGTACCCCAAAAAACCAAGGTGGATAGATAGA
TCTTGAACAAGGGCACATACTCCTTTCGTGTATAGGAGTATACAAGGTGTGTGTGTGTGTGTGTGTGTGTGTGTCTGTGTGTGTGTCTAGTGTC
CTATATAGCCATTGATTGTGGCCCTTGCTTTCAGGAAATTCCTCAGGACTCAGCAGCGAATAGACTGCTTAGGTCAGATCCTCAGAGCAGGAGG
AAGAAACTCAAAACCTCCAATGGGAGACCAGACTCCAGGAAGGACTTCCCTGTAACAGGGCAAGTTAACGTAATTTAAAAAAAAAAAAAAAGAC
ACAAGGGGCACCCTTGGCATCCTGAGAACCTGGTGAAAGAAAACCCAGTGAAAAGAAAATGTGTCTCCCTGGGGAGAAACCAGCCCCACCCCCA
TGTAGGTTGCCCCTCCCCTGTGAAACTTGAACTAAGACAGTCTTGGGGCAAAATCTCTCCCCTAGGTTACCATGAAATATGTATTTCATGCAGG
CTTTCAGGACGTCAAGCCACCCTTTCTCTTGGCATTCTGGGAGTTTCCTGTGGCTTCACGTGTGCCCAGAGAGGAACTCTGACCATGGGGGGAA
ATTTAATAAATGTGGTTTATCTGAGCTCCGTCTCTGTCTAACTTTGTACTAATGACTGGAGTGGTTTGCTGCTTGGAGACCATTATTTTTCTTT
ATTTGCTCTAGCAGCGAGGTTCTGCCTGAGGGAGAAAATGCCTGGGACTGAGGCACAGACATTCTTGCTCTTACCCCTGAGAAATTTGGGGTAT
TCTCTTGGAACGTTTGATTCCATTTCTGAAGCTCTGTCCAGTCACGGAGAATTATCCCTCACTGCGGCAGAATCCATCCCTAGCTGGAATTCCA
GCCGGCCAGCAAGGCCAGTGCCCGCAAGCTCTGTGTAAAGGGTTTCCATTCTGTGTAACTGAAAGCAGTCATGATAAGTGCTAGCCTCTGCTGA
ATGTTTAAAACAAGCTAGGATCTCTGCAGCACACCTTCTGAGCTTAGTTTTATTGAAGCCACATTCTGTAGATGTCATTTTTCGCCCAGTATAG
ACGAGGGACATGTGACCAGGGGAGCTATACCCAAATAAACTTGTTACTTTGAAGTAGGAGAGCTTAGACTAAGATGAATTTGTCACTAATATTC
TTGGAAGATGGAATGTTGCCTTCGGGTGGCTGTGTCCCTTATGTAATCTACATATCTGCACTTTCCCATATAGCAGTCCCTAAATGGTACCCAC
CACCTCCTGGTTTTCACCTAGGAACTGCTGAGACCTAGAAGTGATAGGTACCTAAGCCAAAGGTCTTATGTCCCATAAGAGGTGACTTTGGCTG
CCCTGCTTAGACCCATATTTTCTGAGTATTCAACCAGAGCATCCCTAGCTAAAGGGGACACCCCTCTACTTCCCAGCCCCTCCTTGACTGGAAG
CTAAGCAGAGGCATGGCTGACAAGCCATTCCCAGCCTGACCGTCAGTCAGTTCCCTTGGCCACATGTCGCAAGGAGAAGAGTTGTGCTTAGAAA
GGCTTTTGGTAGCCTCAAACACATGTGTTGAGCTCATGGCGTCCTTTGTAAAAGCAGATCCCAGCCCTCATTTTGAAGGCTTAAAAAAAAAAAT
CCCCAGGAGAATCATGAGAGGAATCCTAAAAATGCCGCCAATTTATAAAATAGACTCTTCGGATCACTTTAAACAAAAGGACAGATCTACTCTG
AGGAAAGAGAGGAACATATGTTTTACATCCAATCACAACAGAAAAACAATCGTCTATCCCAAGTAGCTCCACTGGACCTGGTGCAGAGACGTTT
CCATCAAAGCTTCCGTTTACAACACCACACACCAGCGGGTTGACATCTGCTGTTCTAGACACTGACAAAATTGTCTGTTATAGTATCGACATCC
CAGAGGTGGGGTCCCACAATCCACGCCCCACGCAAGGGTCAGCAGCCTCCTACTCTGGCCCCATTTCTAAAGTTTCCTCTACATCTTAATTCTA
ACATGCTGGGAACCTTGGCTTTAACACACAGGCCTTTTGGAGAACGTTCTAGATTCAGACCGAGGAAGCTTGATGTTTCTGTTTTTACTTTCAT
TTCAGGTTCACTGCTGGCCTATAACACAACCTCCCATACACACCAGTCCTCACGACTGAATCTCAAGGAAGGTGAGTCTGTCCTCTCATGCGCC
CAGGTTCCCTCCACTCTCACCACCGCACAGGTCCATACTTCTCAGACAGGTGGGGGATGCTATGTCTGGACACCAATTCCTTCCTCGGGAAACT
ATTTTTCTAGGACACAAGATAAAACAAAAAACAAAACAAAACAAAACAACAACAACAAAACAGTAACAAAAAAAGCAAACTTTTTTTTTCTTTC
TAAAGACCATGTTTTTTCTTCCTCTGCCACCATTTGCAGAACTACAAGGGAGGCTTTGAAAGATCTCATAAAACATCACAAGAATATAATTAAA
GTCACCCCCACCCAAAGAAAGGGGGACTGTCCTTAGAGAACAATGAATCGGCTCACTTTCCAAGCTGACACTTTGGGGGCTCTCCTGGTGTAGA
TAATGGAATGTATGTCTACCTTATAAAGTTTTGTTTGTTTGTTTGTTTCCTAAAACAAAATTTAGCATTTTCAGTTCGTGTTTAAAGTGGTGAT
CTGAGGATTTGGTCTAGGATAAGTTGACCTGTTCTGGTTTGGCTTCGTTTAGTCAAACAGTTCATTTGATATAATTTGGTTTTGATCAGCGAGA
AAAAAAACTGTCTTGAAAAAATGGTTTTAGTCAGGCGCCACAAGGATGCTTCTCAAAGTCTTCATTGGTAGGGACTGACGCTCCAGCTCTCTGG
TGGAGTGCATGCACAACATGTGTGAGGCCCGGGGTTTGATCCAGCAACACACACACACACACATACACACATAAAAATGCACATACATATAAGT
ACACACACATACACACATATACTCACAAACACGCATATACACATACACACAAACACTCGCATATATACACATACACTCACATATACACACATAC
ACTCACATATACACACATATGATCACACACACATACACACACATATATTACACCCAAACACACAAATAAACACACATACACACACAAATTCACA
TACACACACATGCATACTCACATATACACATACACTTACACACTCACACACACACACAAACTATCACCCAACCTAGTCTTGTTTTGGCCACCGA
GTCCCCAAATCAGATGAATGATATTTTTGTGTGTGAGAATTGAAGCTCTGCCCACTCATTCTACAGTCTGATGATCTCCTGCCCCATCATGTTA
CACATGCCTATAGCAGAGAATACCTATTTGCATGATCAATACACTCAGGTCTGGTTCCCTGGTTTTGTCACATCTCTGTCCTCAGACTCACGTC
AGGTTCAGGGTTCTTCCAGGGAGGGTCAAGTCTTGACTGTAAAGCAATCAGCTTCTGAGGATGGCTGACAATGGGGCCATCGCTTGCAGGTTTA
CAAAGAAACAAATAAGCCACTAAACAGAGCGAGAAAGGAGAGGAAGGATGGGAAGGAAAACAAAGAAGAAAGATGGACGTGAAGGGAAGAGCAG
GAAGGGAAGGCGAGTTTTGAACTATTGAATTAGGATTTACTGTACAGCGTGCTGGTACCATCAGAATTAACCAATGAGACTTTTGGGTAGACAG
AACTAATGGTCTATCTGCTCTGTTCGTACAGGTTTAAATACTGGTTTAGGTTGAGAAACTGGTCTGCAGACAATGCAGTCACTAACCAAGTCCT
CCTTGCTGCTGAGGAGTTCAGTGCAGCCCTGGCCCTGGGCTCTACCTGCCAGGCTGAGCCCCTCCTCCGCTGGCCCTTTTGTGTTGAGAGTGCA
TTTGTCTCCCTGCCGCCTCATTCAGGCTGCTCCGAGCCGCCAGCTCCTACTGGGTAGCAACCCTCTTCTCAAATGAATTGACTGTAGCATCCAG
CCAAACGCAGCACCGGCTGCCCCCATTCAGGTGGTGATGGGCATTTATCTCCCAGCCTCGGCCCTGGCCTCTAGAGACGATTAAGCCAGTGCTT
TGCAGGTGCATCTTTTGTTCCCAGTAAAGCTCTCCACTTAGGCGACTGCCTTCCCCCTTTTGACCTAGTCTCCCTCAAGGAGCCAGCCTGCCAC
AAAATAAAGTACATTAGCAATACAAAGCTAACAAACTCCTCCCCCCTTTCACCCTCTTCCCACCTCTCTGCCTCTCTTCTTCCCTAATAGCTAT
CTTTATTTTTTTAATATTAAATGGAAAAAGAAATAGAAGACATCAATGCCACCTTGACTATGTCTCTCTTTGAATAAGAGGCACACACGGCACT
CAATCAGACCTGGQTGTGTGCCCACCGGTGCCTTTGCTCCCTGCTTTGCAAGCCGCAGGTGCTAGCTGGCATCAGGCGCAAATGGGAGAAGCTG
AGGAGATGAAGTCTATTAATGTATCTTCATGGTACTTATCAACTAGAGTGCTGCTTTAGTTCAGAATGGCATGATCGTCCTTTCTAGCCTGCCT
ACCTCCTAAAACACTCACTGAAGCTATCTCAGGGTTATTTCTATGTAGAACGTAAGACAACATTTCAAAACTCAATTTCCACAGACCTGTAAAT
GTGTTGTGAGTCTCAGTTATACCCAATTGGAACCATTGAACATTTTCTACCTTATTTGTCAAGTTTAATGTACTTAAAGCAGGAAGACAGCGGT
TATTGTCAACCATTTCCATAGCAGCCTCGGCTGTCACTTAAACAGGATTTTGGAGTGTGCCTGGTGCGGGTACATACTTTGTTTTGGAGCTTTG
CACCATTCTGTGAGTCAGTCCGCTGTTTTTGTTTCCTTTCTGTGCAGAGAGGTGGGACAACATGAAAGTGACCCCTGAATCGTTTCTAGCCCCT
CAGAGTATGGTAAGGAAAGTTCCCCTTTCACTTAAGCCACTGTCATAAGAGAGAAATGGTTGTTACCAGAGTCTGGGAAGGCCATGAATGGAAA
GGTTATAGAGCGTGAGGGGCCAGTGGGGACCTCATGCAGCTGAAGGAACAGCTTCTAAGGCAGAGTAGGAAAAGAATGATTGACAGCAGTCAAT
TCAATATTTTCAAAACAGCTAATAGGATTGTGTCTGTGTGTGTGTGTGTGTGTATACATGTTTGAGGGCGTGTGCATGTTTGCATGCGCACACA
TATCTGTGTACATGCATGTGGAGACTATAAGCTGGCGTCAGGTGTTTCCCTGGGTTGCTCTCCACTGAACCTGGAAGCCATCCATTCAGCTATG
CTGGCTGTCCAGTCAGCTCCAGAATCCTTCCTACCTCTCCCTCCTTAGCTTTGGGATTTGACAGCCACACTGTGGCAGGCATATGGCTTCTAGA
GGCTGCCAGGGATCCCAGTCTGGACTCTCATGCCTGGTCCACAGGCACTTCACCAGCTGAGCTGTCTCCCCGGCCCCAGTGATTACTTTTGTCA
CGTGATGGATGTGTCTGTGTCTTTCACCTGATTGTTACACATTGCAGATAACCTTAGTAACATCACTCTGAGTTCTCAAAGTACTTACAGTCAC
TATATATTTGAAATATTTTAAAATATGTATGATTTAATGGCCCTTATTCCCAAGGTTCCTCCTCAAAAACACAGTCTTTTCCCTCTAGCTATCT
TTTCCTAAGTGTAATTTGGTGCAGAAATTTGAATTTCTGCAACATTTTTCTCTGCCTCTGTAGTTTTTCACTGCTGCCTCCAGCTACATCTCTG
TTCCCTTTTCAGAAACAAATGTATACCCAGCCTCTACTACACCTTAGCCTTCCGTCTTAAGGGCACAGAGAGCCCCTTGTAGACTTTGTATTAC
AATGACATCCAAGGACAAGTCCTCTGTAACTTCCTTCTCTGGAGCAACCTGGAGCCCTGGAGCTCACTGGATGGAGATGGGGGCTGGCTGCCAG
AGGCTGCAGGTAGAGACAAGTGCTTTGCGTTGACTCCTGACAGAGAGAGAGTACTGAAGCTGGAAGCTGGTGACGATTTCACTGTCTTTGAATG
TGCAGAGTGTGGCTAAGTTATACACTTGAAAGTGGTTAAAACGGTAAATTTTATTTTTAAAAAATTAAATGCATAAATAACAACAAATACTGAG
CCTAAGGCCCAGACATAATGTGTTAATTCTAAATCTCTACTGGGTACCTACGGGGGCCTTGGGGATTTTTTTCTTAAGTTCTAGCTGGACAAGC
CCTGCTTCGTTGTACTGAAGGAACGTAACCGGCACATCCAGGGCCACTCCAGGTCTTTCTGGATCCTTCTGACCTTCTCTGTCCCCAGGTCATC
CTAGACAAAGACCTGTGGATATACTGATTCTAAGCACGGGTGGGTGAAATATCTCACAGTTGATGATGTCATTATTACTGTTATTGACAACTAG
GTATGAGTTTAGCATCTTACATCTTAGAAACTCCCTGCTCCCATGCCTTCATTTGGCTCTCTAAAAACAGATCATGTGTTGCTAACTTGGGTAT
ATGGGGGAGCACTGAGGCTCAAGAGACTAAGTGGCCTTTCCACATAAGCCTTCTAGAGCCAAGCTTGACCCTACTCCCTTCTTCCCTGTCCTGC
ACACTGAAATCCAGTGTGACTGCTGATCCTGGGTGGTAGATCCAAGGTCCCAGCTTTCTCTTTGGTTTTCTGGGCAGCTCCTGCCCCAGGGCCC
TCTGCAAGTGCTCCTTGCCCCCACAATAGGCAAGAGTAGGCACCTAACTTCAGAGCTCCCCTACATGCCTAAGGCCCTCATTTCCTTAGCCCTC
ATCCTGATCTATCTCAATACCTCCAGCTCTCCAACAGGCAGCGGATATAGAGCTCCTTGTTCACTAACTTCTGGACCTCATAGCTGACGAAGGC
CTCCCGGAGCTCTGAGCTTCTAGGCCCCCACCTAGAGCCTTTGAGACTTGATGCCGAAGGTGAAAGACTGAGAACCACTGCCCTAGCTGCCAAG
GGTAGATGGCCTGCAGAACCTCCATTACTGGCACTTACCTTGACTGATGGATAAAAAGTCTGTCTTCATACTTGCCAAGGATTGAGAAGAGAGG
GGCTAAATCGCCCCATGACACATATGCTGGATGCCCAGCTGCCCATTTATGGATCATGCTTAAGAGAGCCTCTCCTCTCAAGAATTCCAAGGAC
AAGGGCCAAGCATGGAATAGCAACCCTTCWGTAGTATCTACTTACCAAATATTTTCACATCCATTATCCATTATCCACACACCATCACCATGAG
ATCGCAGCTGCTACCTTCTTGTCCCCGGTCCTGCAGTTAGGATAATAGGGCCAAGAGCTCCCAATGTTCCTTAGATGTCACCCATATTTGGGGC
AAAGCAAGACTACCCGTGCTCGCTTCGGCAGCACATATACTAAAATTGGAATGATACAGAGAAGATTAGCATGGCCCCTGTGCAAGCATCACAC
ATCGCAGCTGCTACCTTCTTGTCCCGTCCTGCAGTTAAGGATAATAAACCAAGAGCTCCCAAAAATGTTCCTTAGATGTCACCATATTTGGGGC
CCCACATTTTAATCTCAACATCTTAAGTATTTGTTAATGAATGTTTTCCACTCACCTAGGTAAGCACCCAGCTCCTCTTTAATGTGTTATACTA
ATGAAGTTGCAGTGTGATTTTGGGCAGCGGTTTGCCTTTTCTGTCTCAGTATATTATTTGTATGGCTGTCACAATACCTGACAGAAACAACTTG
ACAGGGGAGATAGAAACTTTGCGGTTTTGGAGAGACTTCAGTCCATTGTGGTGGAGAAAGCATGATAGAGGGAGTGTTTCGCAGAGATGAGAAG
AATAGAACATTTGGTAGTGGGCGATCCCATGGTGGTGGACCAAGATTCAATGGTTAAGCTATAGCCTTTATCATTAGCCTACCCCTAGTGATCC
ACTTCCTCCAAACACTCTCTATCTCCTAAAGGTTCAATGAAATTGTACCATCCAGTGGGCAGCGAGCATCCAAAAGCTCGGAGCCTGTGGGAGA
TGTTTCCGATTAAGTCAGAACACTCCATTTCATCATGTATAAAGTGAGGCTAATTTCCATGCCCTTGTAAATCAGCATGATGTCTGACATGCCA
AAAATGCTTGATAAATTATATTCATAACAGTTCATTATTATGATTTATGACCCGAATTTAATAGTAATTAACATTATTGTTGGGGAAATATATT
TGAGGGTTACCAGAGGCTAACAATGTGAAGATATTGTACTCTAATGGATGGTATAGCGCTGTGCAGGATGCTCCACCATAGCTGAGAAGCCAAG
CTGCTCAAAACCTTCCTAGGGAGAGACCCCTATAGGCGATGTCAGGAAGTGCTGTCAAATCACATTCATAAATGCAAAGTCACTTGTCACATTC
AAGATCTCCAAGGGGAAGGAAGGAAGAGCCACAGTTGAAAATACCTACTGAATATCTTTTATACAAAACAAAAAAACAAAACAACAAAAACAAA
ACAACACCCCCCAAAAGAATCAAACGCCATAAAATGGTTGAGACCAGCCAAAGCCACCCTAACCTGAAAGATACTACAGAAGACTGAAAATACG
TCAGACCACAAGCATTTAGTAWATGAGAGATCCAAGCTGTATAGGACTTTTTAGTATTATATTAGTTTACCTTTTGTGTGGTATGGGTGGTATG
CCATAGCTCAAGGGCAGAGGTCAGAGACAACACATGCAGAGTTGGTTTCTTCTACCATGTAGGTTCTGTGATTTGAACTCAGATCTTCAACGTT
TAGTAACAAGTGACTTTTCTCACTGAGCCATCTCCCTGGCCCAGTGTGTTCATCCAAAGGATGTAGGTGGTCAGTGCCTTAGTAAAGAATGGAC
CTGAATTCTGTGAGTCGTTCTGCCTTCTCCCAGTTGCTCATTGATTCTCTGACTTATGGAGGATTTGCTCCATACAATAAACCTTCCAGAATCT
TGGAAATATGATAGCCAAGGGGCGCCAATCACACCTGCAGATGAGCAGTAGAGTCTCAGAAAGTCGTCCTGCTGCCTCCTGCACACACCCTCTC
CATCGGAGTTCAGAATGACATCATCCGAGAGGTTATGGTGTAATGTGTGTGGGGGTGGGGGGAGGGGTGTATTCTGGAAAAGCTGGATGTGGCT
ATAGAAATGTAGGAGGGGCAACTCTGATTCTGGGGTGTGTGTAGGCACTGCCATATGCCTCCACGTGTGTTCTGGGTGGTATTTTGAAAAAGGT
GAGTCCCTATACACGTGAACAAAGTGTCACCGCGTGCCATGTGTGACCCTTCGGTAGGTCTCCAGCACTGGAGACAATGTGCTTAGGCACTTCA
TGGTGAGACTGGAATCTCAGGTATGGAGCCCAAAGCAGGAGCCAGGAATTTGCCTCAAAATACTAAGGCCCAGTGGTAGTGGGGTGGAGCTAAA
GTGACATTGGCCATGGCTTTTAATTGTTCAGCAGAGCAATGGGCCCCAGTAAGGCTTCACTGGACTTTTTCCCTTTTATGTATCTTGGAATTTT
TCTCTGACTCTAAAGACAATGATAAGTTTTATCTGAGTGGGAGGACAGTTAAACGAGCAGAATGTCTGCATTCCACACCTGTGTACAGTGAACG
AGGGTGGCGTGCTCTTGGGAGGCCCACATTCAGACTGTGAGTCTATGGGGAGGGGAGAGTAGGTGAACATAGAAACAAGAATAGAAATGATGTG
GTGTGTGTGTGTGTGTGTGTGTGTGTGTGTGTGTGTGTGTGTGTCCCTGTCTGTCTCTATCTGTGTATGTCPGTGTCTGTATCTGTGTGTGTGT
CTGCCTGTCTGTGTCTGTGTATGTCTGTGTCTCTATCTATGTGTGTGTGCTTGTGTGTGGGTGAGGGTGTGTAATTGTGTGTTTCTCAGCTATT
TCTGTGGTCCAGAGGCAAGATTTGTGATTATAGATAAACAGTAGCTTTCTCTCTCTGGTTTGTATTTGGTTTTGTTTTTAACTTAAAAAAAAAA
TCCAGAGTTAATGTCTTGTCTGTTTCCTATCTGACCCTCTTTAAGAGATGTGTTTGATTTTTAAAAATTTTTATTTTTACTTACTTATTTTCGA
GAAAAAGPGCCACAGAAAGAGAGACAGAGAGGCAGAGAGGCAGAGAGAGACACACACAGAGAGAGAGAGAGATTGAGAAACTGGAAACAAAGAA
TCGTTGTCTCTGACCCCTCCCACCCAGGAAACCCCAATCTCAGGGGAGTCCAGCCAGCCAGCCCGGCCCTGGAGTCAGACTCCAAGTTTCTGAA
TTCAATTTGACAGCTTGTCTGGGACACTTTCTAAGTAACCATTATCCTTCCTTTCTCTAAGTAAAAGAACTGTTATTCCTGTCCCAGGGTGTGC
TGTGTGCTCTGTTATTGCACACGTGTGGCCATACGATCACGAGCACACGGTCTCTAAAATTACTGGAAACAGTTTTCATGTTCCCTAAGACAAG
TGTAAATTTGTTCCAGGCTAATGAGCAAAAGATTTCAAGTCTCATTCCCCAACCTGGTGAGAAGTAGGTAGATGGGCGACGGCGGATGAGGGGA
GGGGAGCATTTGCCCTGGTGTTGTGCATGTACACACTATTCTTACTAGGATTTCTTTGAAGTACACACACACACACACACACACACACACACAT
CCTTCACTCACCACTATCCTCAGCGTAAGCATAAGTTCTCTCCCTCTCTCGTCCACACTCACATCAGCCTAGAGCTGCAGGTTGCAAGGAGATG
ACTCCTCTGTGTTCAATCTGCAGGCCCTGGAAGCAGGACCACACTGGGTTCTTTGGATCTTTCACATGCTGTTGTGAGGGATTAGGCAGCAGCC
ACCCCCTCTGTTCATACTTGAAATCCAGGGGCAACGTGTAAGTTTATGTCACAGCACCTTCTGGACAACTGTGGATGCTTGCTCTCTTGGGTCT
GCCCTCGAGTCTGAATGGAACTGTACCTACTATGCCTTCAGTCAAGCTTGGGACTGTCTCTCCACTTACCCCACCCCTACCCAGGGACCTGCCG
CCCCTTCAGAGGCCTTGGCCCTATAGCTTTGCCTGGTTCTAGTCTCCTCTGCCCCTTTTATGAGGTGTTTTGTTTGTACCCACTCTTTCTCCAA
TTTAGTACTGTTTAGTATTGGACAGATTCCAGAGGACAGTTTTTTCCTCTCAAATGACTAAAGCACCCTGGAGCTAGAAACCATTGGATGGGCA
GATGAAAGTCACCCTTAACNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNCTTCCCCTTCCCCTTCCCCTTCTCCTCCTCCTT
CTCTTPCTTTCTTTCTTTCTTTCTTTCTTTCTTTCTTTCTTTCTTTCTTTCTTTCTTTCTTTCTTTCCTTCCTTCCTTCCTTCCTTCCTTCCTT
CCTTCCTTCTTTCCTGTTTCTATCTTTTTTCCTTTATAACATATTTATTAAATACPTAGTCATGGAAGATTTTTCTATGCCATAAAACCTTATT
AGTTTAATGCTATCATTATTCCCATTTTATAGATGAGAAGCCCAAATCACCATGTTGGTTTGAAGCAGGGCAGAATCTGTTCTCACAGTCTCCT
CCCCAGCCGAGGTAGAACACAGGCTCTGAAATTTCCTCTTGTATTTCGACCCAACTATGTCTTAATTACTACTGATGCTGAGGGGGAAAAAATG
CCTTGACAAAAGCAACTTGGGGAAGTAAACGTTTCTTTAAGCTCACAGTTTAAGATTCATCATGGTGAGAAGTCCTGGAGGTAGGAGTTTGAGG
CAATTGTTCACATTGCATCTCCAGTTAGGAGTTAGCAGAGAAGAGCGACTGCTCTGCTCCACTGCCTTTCTCCTTTTAATCCAGTCCAGGATCC
AAGCCTAGGGAATCGTGCTGCCCACTTCTACCTCAAGTAACCTAATCAATTGTTCCTCACAGGCATGGCCAGAAGTTTGTCTCCTTCATCTTGC
CAAGTTGACATTAATAACATCACACCATGCCAAAAGACCCATTTCTTTCAGGAAGCTGGGTTTGTCTAATTGTTAAAACCATGGGACTGCATCG
ATTCCTGCAATGGTTCAAAATTTCTCAATGTTACCGGCATTTCTGGAGGCATATTAGTTTGTTTCACAAATTTTTCTGTAGATATATTTTTATA
ATTTGCATGATGATACATGATTAACCTTTTGAGCTTAATTGAACACTTAAGAGAAATGAAAAAAATGAGCTTCAGTAAAAGGCATATTACACTG
AGTAAAACCATTTCCATGTGTTGCAATGAACTTAATCTGAAAAAACGAGAGTGAGACACAGACATGTCATTAGAAATCACGTTGTCATGTTCCC
CTTGGTTGATATGAGCATCAATCATGAAGACCTTCCAGGATCCTACCTCACCTAAGCTCCCTCCATGAAGTTAACTTTTCCTGCACTGGGGCAC
TGAGGAAGGAAGCTGTAGCTGACTGTTGGGTCCTTAGAATCGACAGCTTCCATTGCTTCTGCAGTGTTTTCCCACCTCAACCTTTACAGCCCAC
ACTTTGTTCTCACACTTAATGACAACTTTAGCTTCTTGGTTTACATCAGCATCCTTTCTGTCACCATAGGCTGGCCTCTCATGTCCCTGGGCCA
GAAGACACCACTCCCCTCAGATAGTCAGAAGTTCTCATTTAGATCAACATGACTTTCTTGTCCCTGTCTTTGCTTTTATGCATTTCAGAGCTGT
TCTTAGAGGCTTCCAGAAGAAATAAATGAGGCTGTAGGTACCCATGTATGTGTTCTGCGCTGCTAATGACCGTAACGGGGAATGCACGTCCCCT
CCTGCATCTCTATAAAGGAATGTGATTCCTGTAAACACTTGAAGCTAGAGGGCCAAATGTACAAGGCTGTGGGAAGATATTTGTGGGTAATGAC
ATCCTGGGGAACTTTCCAACCTGACTCCATTCCTCCTCCTTCCATACCAGCAGAGAGAGGCCTTGACTGTAGTGCTCTGTGGCTGAAGCTCTGA
CCATATCACACATTTCTTGGTAGCAATTCACACTTGCTAAGTAAGCTTGGCCTTCCTTATTCCTCTCCAGTCTTGCTGTTACTATATCAGTCCT
GCCGCCTCTACCAAGCTGCTTCTTGCGTTAAAGATCTGTCTCACTCCACACAGTGAAGCTAAAACTCAGAATGAAATTCATTTTAGTTCTTAAA
CATCTTCACCATGTGCTCTGATTTGTGATGGAAACTAGTGGTCCTGTTAGCTGATGTTGAGAAGGGTTTCAAGTTAACTGGCAAGAAGAAAAGG
GGATTGTCTACAAGTGCTGTTGATGCTCACTGGTGGCTACCTCAGCACAGGGTCGTCCAGATACGTGGGCTTTGCCCAATGTACTGATTTAGCT
ATTCCAGGAGGTTTGTTTACCTATACTGGAAAGTATAGCTGGCCTCTCCCATCCAGGCCTTCCACATCGGCAGTTTTGACCAACCATTGCTAGA
CAAAAGAGCATCCTCTTGGACAAGCAAGCTCCCTGTCATTTCCTAAATGACACAGTATAGCAACCAATTACATTGTGTTAGGTATTTTAAGTGA
TCTTAGATGTTCCTCCAGCAGTGCAGTGAGTTAGTGTGCCTGACTTACACAGCAGTGTGTATGCATGGAAGTGATACCCCAGTCGTGAGATAGC
GTAAGTGGTCCATCAATGACTGGAAGTCTTTGGGAGGATGATCATGCGWTCTATTTAGCACTGTACCATGAGAATTTGAGCATTATGGGTCATG
GTGCATGATAGAGACTCTGAAACCATTTACCCTCACATGCAAAGCAAAGACTATGCTTACAAAAAAAGCTTATTTTCCCAAGCCTTTATCTATC
TTATGTCTGTCTGTCTGTCTGTCTGTCTGTCTGTCTCTATCTACCTACCTACCTACCTACCTACCTACCATCCCCGTAAACAAAGCTATAGATC
TGTCTTTTTTTTCCTCTCTATTTGAAGAAGGAAGAAGAGAGAGAAGCACAGACATGGAAGCCCCTCTTGACGCACAGGGAAGAAGTGTATCTTC
TACTTAAAAGGATGGGCTGTAAGCACCACCATTTATTATTTTAATATGAAAATCTTTTATAGATGTAACAAATTCCTCTCTTTGGAGAAGCCTC
AACTTAATATCACAAAGAAATCATTTCTCCTGTAGAATCCCCACGGAAGAGAAGGTAAAGTCTTTCAGGAAGAAAGTCTGAGGCTGGCATGGGG
GTGTGAGGGCTTTCCCCTCTTTTGGGAATCCGCAGGTCTCACAAGTAAATTCTGAGACGCTTCTTCCGCCTCTTTGGTACAGCTTGAATTCATG
TCAGGCAGAAGCCACAGAGAGAATAATAACATTATGTAAGATAATTACTTATATGTCAAATCCCAAATCAATCCTACCTGCTCAGTGTGTTTAC
CTTACTCCGACGAACTGTGTCTCTTCCCTAAGCCCTGAACCCAGGAGCTATTTCTGAACCTAATTGCAGAPTATCTTGTCATCTTTAATTGTGA
CTTTTGTCCATTATTCTTTTCTCCCACCCCAAGATTTTCAGTALATAACCTTGACAGTTGATGGATTTGCTGGGAAAGAAAAATCTAATCTTTT
TCAATTGAGTTAAATATTTACTCAGTCTTGCTCTACTAGGAAGAATCAGAAGLAATTATCTTAGCACGAAAGAAAAAATTTATTTCTAGCCAGG
CAGTGGTGGTGCACACTTTTAATCCCAGCATTTGGGAGGCAGAGGCAGGTGGATTTTTGAGTTCGAGGCCAGCCTGGTCTACAGAGTGAGTGCC
AGGACAGCCAGGGCTACACAGAGGAAACCTGTCTCAAAACAAAAAACAAAAAACAAAAAAACAAAAAACAAAAANNNNNNNNNNNNNNNNNNNN
ACCTGTCTCAAAACAAAAAACAAAAAAACAACAGGAAAAAATTAAGTTCTTATCTCCTATGAATAAAAAAACCTGGAAAAGAATAGTTTGGGGA
AATGTCTAATGGGTAAAATACTGCACAAAGGTGAGGTTAAATTCAGGTCTGCGTTACCCACATAATTAAAAAAAAAAAACTGGCAAGGTGGCAT
GCATCTGTAATTCCACCCTGAGAATAGAGACAACTTGAACCTAGGGGCTCACTGGTCAGCCAATCACTGGCCAAAAACAGTAGCTTCAGGTCCA
GTGAATGACCTTGTCTCAAAATAACAACACTAATCCTACTAAATACCAATAATGATATAGAATGCAGTAAATAGGAGGAAGATACTCAAATACC
ACCTCTGGCATCCACATGTGCATACCAAGATGAGTGTACAAGCACACACACACACACACACACATACACACACACACACACACACACACGCACA
CACGCATCCACGCACATGCATACACACACACACGCACATGCACACATACACCAATGCTGACTGGAAGAAGATTGGACTACAGTTCCATAGGAGA
CTTTCGCGTGTGTGAAGGGTTTGTGTATACATGAPCTWATCTATTCTCCAAAGACCCTTGTGAGGATTAAATATACTGTTTGCTCTTGCATGGT
CTGAATTTACAAATAGTCAATTCTTTCTCACAAGAGTATACTTTTCAGAGAGGGGAAGTCACTCACCAAAGGTGCACACAGCACATAAGCTGAT
GATCTGGGAAGCAGGGTGCAGAGTTGGAGTGACGGACATCCCGAATTCTTTCCTTTGCAGTGAATGGCTTTACTCTTTGGAAACCATGAGATTG
CTTCCCCCAAATAGCTTTAAAGCTTTAAATGACACCCCAAAACAGTCCAGTTCTTGATAAGATTCAGTTATTCCAAAGAAACACAAGCTTCTCG
GGATATAATTTTTGAAAACTCTTTGAGCCTGAAGCGTTGGCTGGCCCAGTTACTCACCAGTCCTGCCCAATAGCCTAAGATCAGTTTAAAGAAA
GTCAGTTTTGATAGTCTGAGTCATATTTTCAATTTCTAATAAAATTCTTACATGGCCACATACCCTGTCTCCCAGCTTTGAACCTAGGGTCTCA
TGCTCATTTGCATGTCTGGTTTAGATCATGTCTGGGATCCCTTGAAGTCTGAAGCAGAGCTTGCCATTCTGTGATGTAGTGGGAATAGATTCTT
ACCAAAACTAAGGCCATTTTCTCAGATACTCGGATGTAAGATTCCACGTTTGACATTGCCTGCTCACCTGCCAGACCAGAACAAAGTGATGCAG
TCTCCCTTAGTCTGACCTCACCTGCCCACTTCTCCTATGATGACTCCAAAGACATGTCCCCTGGAACCCACTTTAGCATTTTAGCTGTCTGTCA
AAAGGCCAGTCTCGGGAACTGCTGGTGTTCGAAGTTTCCTCATTAGAATAGTCATCTTAAGTCATGGATGGGGGAAATCAGACTGACTCTGAAT
AACACGAAACCAGCAAACAATCAAAAGATGTTGTAGAGCTTTTAAATGTCACTGTCCTCCAATATCAACCCACTCATACTACATGTGAAATGAC
AAAATCGAACTAGCTGTCCTGAAAGTTGTCACTGTCAATATGGGACAMCCCAGTACTTTTCAGAGCTGAAACCATGGAGTTCCCTAGAGCTCCA
ATTTAATCATGTTACACAGTCCAAATCTTTGCTCAAATTCTGTTTTCTGAGGCCCTCCTACCCAGCCCTGCCTACTCCCTCTTCTGGCTTGCTT
TGCTCCCTCACACCCAGCAGCTTCTTCTAATCTACTGCATTGTTTACTATTTGTTCTGAGTAACTTCCACAGAAGATACCGACTTTGCGTTGCC
AGAGTTGTATTCTAGGCACATACGTGGGTCTCGCAACAGAGCAGGCATTCAACACAACTTCATTGAATGAGTGAGTATGTGGTAGGGTCTGTCC
ACTAAGTCCAATAAGCTAGTACTCCGGTGGGGCAAGAGCAAATTTGTTGAGGGGGCAGAACTGACTAAGGTCCGGTCCCTCTGCTGCAGAGATG
CTCACCTAGCCAGAGAGGTAACACACTTGACACTGGATGGTTGAGCAGACAGGGACAGTAAGCACGGAGCAATGGCCCGAAGGACTGGAATGAA
AGGTGTGCAAAGGTCAAATCGTGAGGCAGATGCAGGATCCAATCGCCCATGGCAAACGACGACGTCGTATAGGCAGCGTGCCCTCTCTGTGTGC
AGAGCCAGAGGGCAATCATAAACAACTCTTTATTGCCTAAATGAGCTGTTGTTTTGCCTTTTTTTTTCCTAGCTGGTTTTAAACCTGACATCCA
TCTGACCAAAGAAGTATGCGATTGAGTCGTGTCTACTGTAACCCAGTGTGGAGAATTGACAATACCCAATTCAGCTAGTCAATCTGGCTTGCTT
TTCCTCTTCCACGTATGGCTCAAAGCCCCACTTTGAGGCCTTATCAAAATAAATAGTTTATTTAGACATCTGTGCCATTTGGACAGCGAGCCAA
CGGGGCAGACAAAATGAAAGAAGAGCCCAGGCTGGGCAGGATGCCTTGCAGGAACGTAACGAGGCTGTGCCCAGAGAATGTGCCTCTTACTTTG
TCAAACTTTTTGTATTTCAAGCCGCCCCGTGCCTTTCCAGCATTTTCCTCATGAATCTTCTTTTCCCTCTGCACACAGGATTATCTTAGGCACT
ACGTCCATTGTCCTCCGCGCAGTGGTACTGGTCCTGAGCTCACGGCCTGCTTGAATTCCAGGACGAGATCCTTGAGGTAGAGCAAACTTGAGCC
AGGATGTTTTCTCTTGCTTCTTTGAGTTCCAAGCTAAAGCAAAGGATTCCCTTTTTAACTTCCTCTCCCTGCAGTCTTGAAGGATGCCAGGATC
CTCTGTAGCTCTGTGGAACAAAAATCCCACTGGAGGGAAGGCTGGGTGAGCTTTAGGGAATGGATCAAATGAGGTAGAGGATTCATCCTGAGCC
TCTGGACCAGCTAAAGAGGAACCTCAGCACTCCTCAGTCATGGAAGAAGCAAAAGCTGACCGCTCCCACCTGCCCATCGCTGTCTTCTGAGATC
ATGTTTAAAACCAGGGTTCAGGATGACCCACCACTTTCCTTGGAATGTATTCCTGGATGAACTGTTTGAGGGCGCCAGGTTAGAGTTGGTCTTT
GGGGATTCTTGGGCCTCTCTTCCTTCTCAAGGCCTTCCTGTACGACCTTTATGACCTCCTCTTATTGTAGAGGTAAAGGCAAGATGGAACAGGC
ATCTATATATCATGTCCAAAGTTCCCATCTATGGAAACACAATTGATTCCCACAGCAGGTCTGTTAAGATCTGTTATCATGCTGTGTAGGGGAT
GCAGAGTTGAAGTTCAAAAGACAAATAACCTAACCAGGGGATTCAGATGAATAGGGACAGACCTGGGATTAGATTCAAGGGGCCATGGATGGCC
ACTGAGACCACCATTAAGCCCTAGAAAAAAGGAGCACTCCCCAGGGACAGAGCCCTAGAACTCTGTTTAGACTGCCCATTTTCTTCGTAGTTTC
TGTTGCCCTGGATTTAAAGTAGGATCCAGGCCAGTAAGTAGGTAAGATTCTTAGCAATTCAGAGATGATTTCAGATGAGAGACTGTTCCAGAGT
TGTGAACTGGGGGTTCAGGAAGCATAGTTAGCTTTTGTAGGAGGTGTTCAGCCCAAAGGAGAGTCCAAAGCAGAGAATTCCAGTCAGGTTGGTG
GTCAGAAACATCCATGCCTCTACTGAGATAGAACCTACTAGGAGAACATGACCTCGAATAGAGACTCCACTGAGAGGATCATGTAGGCAGAGGC
CCTAGGACATCTGACCGTCCCCAAACACAGATGACACCTTACTTATTGGCATCACTGAGGTTGGCGCGCAGACCCAGGTTTTCCTGGCATGAAC
AGCACAGACACTGCCAGCCTGGCCAGCAGACAGATAATTCAGCGAGAAGTGAATCTTTTTTCCACTGTAAGCATAGAGAGCACGGGGCCCAGTG
TGGTGAGGGACTCAAAGAGAGGCTGCTGTCTGCTGCTGATCGCTCTGTGTGGGTGATGTGTTGACAGGGTGTCAGCTGCGTACAGCCTGATGCA
CCTGCGTCTTGGACAGTTAACTGGAGGCATCAGGAGAAAACAAGGTATTCTCAGAGATGAAGGCCATATTTCCAGGACCAGTTTAAACACTGGT
GCGTCTATGCCATGGTCACAGCCAGCAAAAGCCTTCCCTTTCCAACGGGGCTCAGAGCTCATATTACTCTACCCTAGGAGGCTGTCTTGGGATC
TAAGAGACCATTTAGTGTCGCATTCAGCCACACTGAGTATTGCATAATGGAGAAACTGTTCAGGGCGCCGTAGATGGGaGCAGGATTTATCCAC
AATGGCTGGCAAAGCCCCAGAATCCCGGCATGAGGCCCAGGTCTGTTTGCTTTGGCGAGAACTGACCTCAGGGCTGCCTGAAACAAGAAACCTT
AGGCAAAGCCCACTGGTGGCTGCTCAGCAAAGGAATCACAATAGCATTTGTCATCTTAGGACTCCTTAAAGCCAGGGGGTGTCAGCCTTCCCTG
AGGCAGAAAGAGAGTTTGTCAGAATCAGGGCATAATTTTCTGCTGATAAAGCCATCTGCCACCACAGAGAAGGTCTAAGAGCATCACCTCGGGT
GCTGGCCGGGATTGCCACATTTTTGACACTGAGTCTTTGGCCACCTCAGACTTTTCTTCTCTAAAGCCAGATGTACCTGTCAGGATGCGGGCTC
CATGTGCCACATGTGGGGCGTCTGTATGTGTGCACTTGGCTGCTTGGAGCCGCCAAAGCCCGCGGGAGAACGCTGCACTATCTGGCGCTGACCA
TAGCCAGGCAGCATCTGGCTCTGGAGCCAGGAGTGTCTGTAGGAATTTGTACAGCAGGCTTGCCCCCTTCTCTAGCTTTPCCGAGTTGCTTACC
GATATGTCTGTCAGTGTCCCTGGTACTGTAGCTGACCTCACTGCCAGGCTTTTATTTGCGCGACATTTAATAGGAGAAATCACAATCCCTCATT
TTAACGATAACACTGCCCTGTGAATGATGGAACCAGTTCTGATAAATGCCGCCTCCCTTATTTTTTATCCCCACGTGAATATCAGAATATGCTT
TCACAAACTAAGATGCCGATGACCACCCAGATGACACAACATTACGGGACCACCATCCCTTGTGCTGTCCCCTGTTGATCCAGGCAACATAAAT
GAGTGGTGCCCAGCTCCTGACCTCCCTGAAGGAACATACTCTCAGCTTCCACTTCAAAATGGGTGGCTTGCTGCTCAGCCGGTGTTGACATAGT
CTGTAGTCCTTATCACCACAGGCCTGAACTAGCTAGAGACCTAGTTCAAGAAGGTAAGCGTCTTGGATTCTTTTCTGCTAGCTGACAACAAAAG
GGATTGTCTGCATTTAACAAGGAGAGACACTGCTGATTCCATTCGTTGGTTCGTGTGTATGTATTGCATGTTTACCATGTATGGCTGTGAGGAC
CTGTAGTCCTTATCACCACAGGCCTGAACTAGCTAGAGACCTAGTTCAAGAAGGTAAGCGTCTTGGATTCTTTTCTGCTAGCTGACAACAAAAG
TTAACAAATGGGCCAATGTGTTTCTGCTCCAGGTGTTTTTCAGATGAGTGAGAACAGCACAGATCATCTCAACGCTAACGATATCTTTCTCCTA
AAACATGQGGCACACTGGTCCTTCTGCTCAGTGGTAACTTGACCCTTTCACTTCAGCTCCAPAGGTCTCCTGTGCTTTGAGCATCCGAGGCAGC
TAGATGCCCTTGGCCTTGGGCTTTCTCATCTTGCCACCTCCCTCTCAGAAACTGGTTTTTCATTTCAGTCTTGCACGAAAQGAGACATACCCTG
CACCACAACTCCTCTGTGGTCCATTCACCAGCTTGTGCAGTAAATGGGCAGGCCCTCACACTGCTTTGCAGATTCACAGGCCAGAGAAAGCACC
TCTGCATATGTAGAGGGGCTGATGGGAGAGAAGACTGTATTTTCCATGTTTGATTCCACTCACTGGGTCACACAGTGACTGAAGATGCCTGCTG
ATGGCATCGCCATTCATTGCTCAGTGAACTTCATAGTGACAGTGTTGCAAGGCTGGTTTATGGAGTGGCTTCTCCATCTGTATCACTTCTGCCT
TCACTTATGGTAGGACCGAGTAGTACTGAGTTGGAATCCCCAGGAGATAGGCTTATGGGAGCATCTCCCTGCGTACTCAGCATCCCCCACCTTC
GAGGTCTGCTGAATCTTAGACTGAAAATAGAATCTTAGAAAAGATGGGCTGCTGTGGCCTACCTCAAGCTTCAACTTCAAAACATTATTCCTCA
CCTAGAATTACCAAAGAATATGGTGACGTCCTAGGAAACTAATTCCTACATTCTGGGACAGCTTTCATCCAGGAGGAGGGCTTTCCGAAGCAAG
TGTAGGTTGAAATTTTTAAGGCAGATTTTACAAGTGCTCGAGACATCAGATGGCACACATCATCTCCTGGAACATAGTGGGTCCTTCTCCTTTT
TAGTTACTCACCTGGCTAGTTTCACGCCAACTTGACATAAGCTACAGGCATCTGAAAGGAGGGAACCTCAGTTGAGAAGGATGTAAGTCTTTAT
CTTAATTAGTGATCGATGGAGGATGCTGTAACCCCAATTGTGGCTGGTGTCATCCCTACATTGATGGTCCTGGGTTCTATAAGGAAGCAGGCTG
AGCAAGCCATGGAAAGCAAACCAGTAAGCAGCACTCTTCCATGGCGTCTGCATCACCTCCTGCCTCCACATTTCCTGTTCTAGTTCCTGTCCTA
ACTTCCTTCATTGATGGTCTTGTTACCTGGGAGTATAAGTGAAGCAAACCCTTTCCTCCCCAAGTTGTTTCACTGTAGCAATGGTAAGCCTAAC
TAAGACACTTATATTTATCACTTTCCAGGTCATCCACTTTCTCTTCTGTCCTTACAAAATGACCTCCACTTCTTAGGGCCTCTGTGCTTTCCAC
ACAGCATCTGGAATGTGTGGAAATGTCTTTGCTCTATGTCTCTCAGAGTCACTACATACTGGGAACCCCCTTCCCCGTATCGTTTGAATGATGA
AGGCCCTAAAGGTGATAGCTTAGTTCCTACAAGGTCCCAAAGGGCCACCCAACCCTTCCTTCCTTGTCCTTTTCTTCTCTCCCTCTACATTCCT
GTATGCCAGCCATGCCCACAGTACCACTAGCCTTTTCATGGCTCTCCCCCACCCCCACCCCCGAAGCTAGGTCTTGCTCAAGTCCCTAAGGCCT
CTAGTGCTTCTTGCCTPCCCCTCCTTTGTCTCCAATAGTTATTGACATCTCAACCATTCTTTTGCACATGGTCGTAATACCTCATAGTCTTTCA
GACTGTGTCATGTCTAAATGCAAGGACTTTGTTTTATATGACTGACTTCTCTAGTGCATGCACACAGCACCGTTCAGAATGTTGATTCTAAATG
ATTGACAGGAGACCGAACTAATCATATTCCAAAACTTAGCTGGCTCCCCCTTGCCTGAGGCTTATCCTAAAGCTGAATGCACCCTGTGGGACTC
CACAGATCCCCTCACAAACTGTCCATAAAGCTATCAACCTCACTCCAAGCTTATAACAAGGCTAGTTGACTTAAGCTGTTTTCAGGAAACAAAG
AAAAAAAATAGATCAAAAATGAAAAACATTCAACTTAAAGGAGCTGTGTCTCCTTGTCCTGTTCCCTGCCTCCCTCTCCAACCCCACCCCCATG
CTCTGTTTTGCTTTTCGTGCCAATGGCTGAAGACTATAATTTTAAAAACTGAGCTCACTTTTCCACACCCATGAAGAATGTGCGTGTGCACATG
CATGTTTCCACAACGACACTTCCTGTCTTTTCGCTTGTCTTACCCAGTAATACTCAACCCACCCAATCACATCCATCTGTCCATTCCTCCATCC
ATCCATCCATCCATTCATCTCCCCAGCCCTTCCTTATTCTAATGAGTTTGCGAGGCTCAGGACAGGCCAAAGCGGATGCTTTGTGTTTCACCCT
GTCAGACAGTTGTTTGATCAGGTTTGTGTTCCATTTATTCAGATGGTTTTTATGGTTCCTTTGTTGTTAAGGCAAACACTTTCAAACTCATAAT
TCTGTATCCTTATCCCTTCTGGTAGGCCTGTGCCATGTCTCAATGGCTTTCTCACAATGAGAAACCAAATCACTCTTTCCACCCCGTTACTTGA
GCAGTTTCTCAACATGCTTCTCCAAGACAAAAGCTGAGTCAAAGTTCAGGGTCCATATAGTGCTGGCCTCTCCCATCTGAAAGATCTCTGTCGG
TTCATTCTCTGATCACCCCAACTCCTCACCACAATTCTGGCCTGACCTCTCTGTGGGTGGTTATAAGACCCTGCCCCTTTCTCCTACAGACATG
TCTGAGAGCCTTCGTCAGCCTACAATGTGGACACATGTCTGCATGTCATTCCATAGCACCAGGACTATGAGGTGCGAGGCTGGAGTTGCCCTTA
CAGGAATGTCATGGGCCTGGACCTTGAGACGTTCTTCCTAGAGACCTTCATCTAGACATCACCTCTTAGATTTTCATCCTTCCCCAACATCCTT
CCATCAGTCAAAAGAGTTTGTATTTTTCCCTTGATTTATAGTAGTTTATCTTTTTAGAAAATCTTGTAAAGTGGCTCACAAATATTCTCACTTG
ATCCTTAGCATAACCCCAGAAGGTAGATAATTCATATACAATTTTGCGTCTACAAAGACACGGGTCAGGGGAGACCTGTGGGCTTCCCACGTCT
CACAACAAATAACACCAGCGATACCGATTGACCCAATCCTCCAACTTCAACTTCACGATTCTTCCTACTCCTTCAGCTCCCAGATTACACAAAT
AACATATTACACTAAATATTACTCACCCAACACCATACTTCACCAATCTAGTGCTCCTTAACAACTTATTGCTGAAGTAAATTTAACTTCTTCT
GCCCCCCAACACAGCATCTTCATAGAACGAACCTTCCTCCAAAACATACAGACCTCATCTGTCCTCTTTTGTTCTTTCTCATAGACCCTTCTTA
TCACTCTGTCACGACAGCACCATGCAACAATAATATCAACTCTCCCCTCAACAAAAGTAACTAAATACCAAACCATTTGGGCGTGAGTCAAATG
TTTCTCCACACCCACAAGTTGGGGTGTGGGCGTAGCCTCATAAGACACACACTAAAAACAACTCTTCCTCTTCATCCTCCCGGATTAGATGTCA
GTGTGAATTTGCAGTGCCTGCTCAGATTTTTGAGGTTTATTTTGTTTTTTTTTTTTTTTTTTTTTGCTATATTTATGTCTACTTCGTTTTAGAA
CTTATAAGGTTTGAGGTTTTCTCGGGACAACTTGATTTATAAAAGAAAAGTAACTCGTCTGGATTTTGATATTTCACTCACCACCTTGAATCTC
TGGGACATATATTCTCCACATGACAACCCTCAGTCACGTCAATACCCCTAGCCCTAGTTAGAGAGATTAACATGAAGGATAACCTAAGGCATGT
TGTGCATCACAGACATTAATTTGACCAACAGTGATCAGCCGATGATTTGTTGAAATCCACCCAGTATCTTCCATTAACCCGCAGTGAAAGAGAG
CCATTCACTCTTCAACCAACGTGTCCATNNNNNNNNNNNNNNNNNNNNNNNNNNNNNNNNNNTATCAGTGTGCAGTGTTTGTTCACTCTCCATC
CAGGACATGACGCTATCGGACTCACAAAGTATAAGCCAGGCAAGGACCAGTTATTTAGGCAGGAGAGAGAAATCATGGACACACATATGTCTTC
ATCTGCCTTGTATTAAACACTTCTTCCCCACCTCCTACCCTTAGACCTACTCAGACACGCCATGTCTAGGGAATGTCTAGGGAACAGGAGACAC
GTAAACCTGGAGAGCTAGATGTCAGGGACGACCATGGTGGTATGTCTGACAGTGAGGAACCTAGGTTTATGGAGTTTAGGATGCAGACTACACC
AAAACATAACAAACGCATACTGAATGATAATATGGAACTACATTCCACATCTTCAGTCCTAAGCAGTCCTGAACACTATGTCACTCAGTTTGAA
GTATCACAGGTCCAGTTACACCGAGCCACCCATCACACAAAAAGCAGCATACATTCAAAACTGGGGAAGCCAGAGGTCAAACATGAACAGCCTT
GGAGAGGCTCTCACGAAACACAGTGTGACAGGCACCAGGTGGTCTGTGCTCAAATGACACTTTCTCACGAGACACCTCTGTATTGCCATGTCTA
TTTGTGGGGGACACGTGTGTACATTTGGGCCCACAACTATAGCCCTCTACCCATCCACATACATCGACGCCAAACACACACACTCGCTGTTCTG
TTTTATCACTCTGTCCCTGCTCCCTTTCACACACTCTCTTACTAAACCCTCGAGTTCACCATTTCTCGGCTACCCTGACTGTCCATCAACCCTC
GGTAATCTTCTGATCTGTGCCAATCCCTCTCCGTTCCCACGCACGTGTTAGAAGTGCACACAGACATGTCAAGTTTTTATACGCCCATCCTTGG
GATCTGGGCCCATGTCCTCGTGCAGCAAACAGTCTTACCATGGACCCATCTCGAACCTCAACCTCCTACATTTCAAAAGAATGGTCTAACCCCA
TGTCAGCAGACATTGTTCAGTGTTTTCTCTTCTGTTAAAATATAACTTCATTGCTAACTCTTCTGCCCCGAGACCAGTGCCAGCGTAGCGGGTT
TGATAGGTATTCAGGAATAAAATGAGTCTCTGACTCCATCGCACAGTCTGCTCCCCCCACCTCATGTCCCCTCCTTCTGGGAGATATGTAAAGG
GAGCATTTTCTGCCACTTTTCTTGATTTCTCCACAGGTCCTCTCCTTGGAGGATCACAGACCATGGGCAAGAACACTCACCACCGGCCCCAGCC
AGGTATGTGATTCAAATATGTAGCCTCTCACTTGTGTGTTATACAGTTGTGGAGTCAGTCAATGGTGGAGCAAGACAGTGTACAGTTGGCTGTC
CATATCTCTCCCTTTTAGGCCTCCGCATTTAACCAAACACAGGCCAAIAATATTCAGGAAAGAATCTACATCTGCACTGACTGTGGACACACTC
GCTTTCTTGTCATTATTCCATCAACAATACAGTACGGCTCTTTGCATAGCATATGTGTCATGATGGTAATCTATGATTTAAGGAATGTGCATCC
ATATGACTAGATTGTATGCAAATAACCCATTTTATGTAAAGGACTTGAGCACCCACACATTTCACTATCCTTCACAGACCTTAGAACAAATCCC
CTGTGTATCCCAAGAGGCAACTCTAGTTCACACATTGTCTCTTCTTTCCAACACACTATCTGGCAATTAACCACCAACTCCTCCAACCACGGAG
CGATCTGTTAAAAGGAACTTAAAACTCAAATGGTCCAATGCCTTATTTCGTGGAAGAAAACAGGGGTTCCGAAGGGTGAAAGGATTTGCTGAAA
CACAAGCAGAGCTGAACTTCTTTAAGTTCCACACCCCCCACTCCTTCTCCAACCATTTTACATTTTACGAAACCATACACAACTCCCTTCACCT
AGTACATACGCACTCAACACATAACACCTTTTCAACCTATCCACCTCCACACCCTTAATAAACAAATCACTTAGACACTCCACTCTATAATCCC
TCCGTAGCATGGCCTCTTATGATGGGATGCCCTTCTTTCTCACCCTGACCCCGCCCCTGCTCCTCCTTGCTTGCTTCAACAAACAGGAAGTTGA
CAACCTACCTTGACTTAATCCTTTTCTGAGCCAGAGCCCTCTGTTCACCTACCCCTCTCCCCACTTGTCTTCTCGCCAGACCTCCCACACGAAC
ACCCTACACAGATCTGAGGAGAGCCATTGCACTCAGCTCTGCTGTCTTAATTAACAATCTGAATTAAGATCTGAACAAGCTATTTGCTTTGGGG
TGTGCAAACATCCTTCTTCATTTCCAGCCAAAATATAGAAAGAGAAATTGTACGAAAAACTGAAATTCAAGTCAAAACCAACTCAGGTACACCA
AATTTTTAAACAAGTTCCAACAACAACAACAAAATTGAATATCTACTCAACAATTTCAGTTTCATCAAAATTTAATAAAAAAGTGTGGACTGAA
AGCCTTCAAGAACTGTTAAATCAATTCTTTTTAATTCATTAATAATTACTATTATAATTGAAAAAAGCAGTGTGTTTTTAAAAAGGAGTGACTT
CATAGCTAAAATACAAATTCTGCTTACTGTTCACATTTTCAAGCTTAAAATCACAGAGTTTTGGTAGAAGGGAAGAGAGCCCACCAGCCCAACC
CACTCTTCATAGCTGAGAAAACAAATACCCAGAGGTTGTCCAAACCATGATTTAGTTCATGACATAGCTGGGACTAGAACCCAGGCTACCTAAT
GGTTTTTATAGTCCATCGGAAAACTCAGACATGCGTAATGTCTTCAGCATTAGAAAGATCTATTAATCGCTAGTAATTGCTGAGGAACCTGCTT
TAAGCATGTTWAATAAGCAATCCTTCAGTTCATTCACTGCCTCCTTGAATTTAGCCAGATGCTTTCCAAACGTGTCGGCGATTTTGTTTGGGCG
CTACTCACAGCATAATCTAAAACTTTACAAACCAGGTATGAAGCAACAGTCCTATTACTCCTCTACCTTCACTGGGCCATAACTTTGATCAAGG
CCTCTAATCAAGCTTTCCTATGGCCCTGAGATAAAAGTAGGAACCGTTGAACTCTCCTTCATCTTGATGCTGCTCTCAGATTATTTGCTGAGGA
ATGAACTAAGCTTTTTTTTTCTTTCTAGATCCTTATCAGATCCTGGGGCCAACCAGCAGCCGCCTAGCAAACCCTGGTGAGTTAACTTGAACCT
GCAAGCCTTTCCCTCCAGAGCATTCCTTATATCTGTAAGGTTACACAGAAACCTTACTCCTCGGTACGGTACAGGCACCTGTCTCCAGACTGCA
TGGGCTTCCTTACTCAAATATTCAACTGTCCACAAACAAGAGGCAGGGCCTGTTCATCACTAGCAGGGTCTGCTGCCACAAGACGTTTCTGCAC
AACAAGCACCTCTGGACAGAGGTTTATGTCCATGGCAAATGACTCACATCGAAGCTCCCCGTTAGGGAACCTATTCTCACTTATTCCTCATCCA
GGGCTCATGGATAAATGTCCCCAACTGTTGTGGTTTTGACTTGAGTGCTATAGTTATCCAGCCTTTGAAAAAGGAAGTGCAAGCATCCCAAGAG
AGTTGCACACACAATGAACGTGACAAGATGTGAAGGATTCACTATTAAAAATAACTCTCGGATGTGTTCTTCTAAACCCAAACCATGGAGATGG
GGAGCTCAAGGCAACAAAAGCCAGGCCCACGTGCCTGCTGCACCTGCAGTGCTACAAGCTCGCTGGGCCTCCTCCCTAACCTTTAGCTAAACCA
TTCCTGCCCAGAAGATAAAAAGCCATCCAGCATCCAGCCCCTCCCGTTGCCTCTGCCCATGCGGAACTGAACACTAAGAGCCGTGCACTTTTCT
GGAAAACAGCCCAGCTTAGTGAACCCCAATTCTATGAGGTGCCTTCTCTCTGCTCTACCCCACAGCCACCCCCCAAGAGGTTCTTGGATACTTG
ACACTCTTTAAGGCCAATGTTCAATACTTTCAGCGATTTCAGTACTAAATCCAGATTAGATAGTCACTCAGGTCTAGGTAGAAACCTCTAAGGC
GAGGGTCAAAAATCTTAACAAAAGGATGTGGAAAGTTGGCTTCCCTGAGATCCTAAGAGCTTGGGACAAAGCCAGCATTCACCACAACCATCCT
TTGGGACAATCTGTCCTTAAGATCTAACCTTGCTTACTACGCTCATTTTGGCTGCGCAGTAGAGTAACAGTATATACTGTACCCTTTTTTTAAG
ATTTATTTATTTTATTTACGTGAGTACACTGTATCTGTCTTCAGACACACCAGAAGAGGGCATCAGATCCTATTATACATGGTTGTGAGCCACC
ATGTGGTTGCTGGGAATTAAACTGAGCACTTCTGGAAGAGCAGTCAGTGCTCTTAACCGCTGAGCCTTCTCTCCAGCTCCGTAATGTACCTTTG
GAAACTAGCTGGATAAGCCGGTCTTTGATGATGAGCAAGCTTTTGAAGTGATGGGTGTGTGAATTGCCCCTCTGTGATTGTCAACCACTATGTA
TGTCTGTCAAAATATCACCACATGCCCTGTAAACGTGTACAATTATCATGTGCCTATTTCTGTTTTCTAAAGGAAAACATAAAGCGTIGTCTCC
ACTTTCCTTTTTTGTTTTGTTATTTGTTTGTTTGTTGTTGTTGTTTGTTTGTTTGTTTGTTTGTTTGTTTTTCCAGATAGGGTTTCTGTGTAGC
AGAGCCCTGGCTGTCTTGAACTCCTAGACCAGGCTAACCCCCAACTCACACAGATCCACTTGCCTCTGCCTCCCAAGTGCTAGGATCAAAGGTA
TGTGCCACCATGCCTAACCAATAAAGGTTGTGGTGGTGGTGGTTGTTCTTGTTGTGGGGAAAGGACATGAGTTGTCATGTTTAGCAATCAGAAC
TGAGACTACTGTTCTTCTGTTTCCTCCTGCATGCAGGGAGTGGGCAGATCCAGCTGTGGCAGTTTCTCCTGGAACTACTGTCCGACAGCGCCAA
CGCCAGCTGTATCACCTGGGAGGGGACCAACGGGGAGTTCAAAATGACGGACCCTGATGAGGTGGCCAGGCGCTGGGGAGAGCGGAAGAGCAAG
CCCAACATGAATTATGACAACCTGAGCCGCGCCCTCCGATACTACTATGACAAAAACATTATGACCAAAGTGCATGGCAAAAGGTATGCCTACA
AGTTTCACTTCCATGGCATTGCCCAGGCCCTGCAGCCACATCCAACACAGACATCCATGTACAAGTATCCCTCTGATATCTCCTACATGCCTTC
CTACCATCCCCATCAACAGAAGGTGAACTTTGTCCCGTCTCACCCATCCTCCATCCCTGTCACCTCCTCCACCTTCTTTGGAGCAGCATCACAA
TACTGGACCTCCCCCACTGCTGGGATCTATCCAAACCCCAGTGTCCCCCGCCATCCTAACACCCACGTGCCTTCACACTTAGGCAGCTACTACT
AGAACTAACACCAGTTCCCCTTCTGGCTGAAGTTCCAGCTCTCACTTTACTGGATACTCTCGACTTAAAAAGGCACAGTACCCTTGAAGAGATA
AGAAAACTGGATGTTCTTTCTTTTGGATAGAACCTTTGTATTTGTTCTTCTAAAAAAATTATTATTTTTATGTTAAAAACTTTTGTTTCCTCTA
CCTGAAAAAAAAAGATCATTCCATGAGCCAGTCCACCAGTTTGGATTCTCAACCTCCTATCATCGAATGAGTTAAATATTTAGGTTACTGGAAC
CGTTTATACCATGATTCTGAGAAAGGAGTACGCATTTTCTTTACTCTTTTTTTTTTATGACCAAAGCAGTTTCTTATCACCACACGGGTCTCAT
CATTGTAGGATTCCCTACGATCATGAATCATGGACTTGACCAGGGTTGGTCTGGPTTGAGACTTAGTAAAAGTCAAGCCACGATGTTTATAATC
TTATCTTAGGAGGACTCAATTCACTGCATCGCAACTCGAACACTCGCTCTCAGGCCAGTGAAGTTTTTTGCCCAACTGGAATTTAAAAGATGTG
TGTCTATGTGTGTATTTAAGAAGCCATTATTATTACAAAATTCCTCACAATGGCCACTATGTGTTTGGGTCACTCTTCTCCTCAGAAATAGTCA
GAATATGAACAAAGAAAGTTTAACACAAACTCAGACACTCCTGACGGGCAGAGGATTAAATAACATTTTTTTGGAGGGTTTAATAACATTTTTG
GAGGGGTTTTTTTGTTTGTTTTTGTTTTTGGGGGTTTTTTTTGTTTGTTTTTTGTTTTTTGGTTTTTGGTTTTTTTTTGTTTTTTTTTTTTTTT
TGGTTTTGATTTTTAATGACAGTGAGTCCCAGAACTTTGAAAAGTCATGGGGATTTCTAAACTCAGATTCGCAAACGCTGTGCGTTTGTCAGAC
CACCAGACCAAGGTCAAACAATCAGAAGGCAACTAACTGTATAAATTATGCAGAGTTATTTTCCTATATCTCACAGTATTAAAAAAATAAATAA
TTAAAAATTAAACAATAAGTAAACGAGTTGACCTCCGTCACAAATGCAGTTTTACTATCAAATCAATCACTGTTATTTTTTTAAAATATAATTT
GTACATCTTTGTCAATCTGTACATTTGGGCTATTTGTACGTTTTTGTAACTGTTTTTTTTTAATAAGCATAATGTGACTATTGAAAACGAGGAG
TTAAAAGTCACTGAGTTTTTAGGAAGAAAAACCTAAAAATACAGTTATTTAACACGCATGCCCAAACAAGATCTGTTTAGACCTACAACGCTTT
AGAAATGTTTGTAAATAACAGAGTTGCAATAACCTGAAAAGGACAAACAAACTTTTCTCTGTGCACACGAGGCACTCTCCTGCTCTATATATGC
AATATATTTTTAGATGTGCAAATATATATATAATTTTTCAGGTAATCGTGACTTTTTAAACGATATTGTTAAGGTGACAACTCTTAGTCCACTG
AAGACTAAGTTGTAAAATAATTTGACCTTAATAAATTGTGCCTTCTTCTTTTTCTTCTTCTCTCAGAACTGGTGACATTCTTTTCTTATTTACT
TCCAGATCAAAGCCTTTCCCGGAGGGAAGTTGTTTTTATTGCTGGTGTGTGTGTGTGTGTGTGTGTCTGTGTGTGTGTGTGTGTGTGTGTGTGT
ATGTAACAACACACAGCACACATTTAAAGGTCAGAAGGCAGTTTTTATCAAATAAATCAGTTCTCTCCTTCCATCTTAGTTTTTGAGGCAGAAT
CTCTTGGTATGTCTCCCACCCTGTGGACTCCAGACACACAAGCTTCCAAAAAGCTCTCCCGCTTCCACCTCCCCTCTTACCACAGAGCACTGCG
ATTGTCGGTGAATACCTCCACGTATGGCTTCTTTCATGAGTTCCAGGGATCAGAGTCAGTCATCAGGCTTACAACGTCTCACGTTTATCCACTG
AGCCCATCTCATTGGCCCCGAGCAAAAAAATAACTTGCCTACTTATTCTTGTACCATCGATAATCTCCTAACTGCATGTGCTGCAGTACAGTGG
AAGACACAGTATTCTGTGAGTTTCAGATGCTTGTTAACAATGACGATTTTAAAGCTTCTCTAAGCCATTAAGATACTGTATGACCTTTGTAAGT
GAGAGACAGACCTAGGTAAANNNNNNNNNNNNNNNNNNNNTGAGAGACAGACCTAGGTAAACTATATCCAGGGAACTACAGCAGCAACCGTCTC
CAGGACCTGGCCAGGTAGACTTGACTCTGTTCCCCACAAGAAGGCGACCTGATGCTTCAGCAGAGAACAGGAGTCTAGCCTGACTTCAACCCCA
CACTAGGGTCTTACGTAGACTCTCTGGCTCTATWTTTCAGCCCAATAGAGAAGCTTGCTTCCAACGGCTTCAGCGTTAAGCATCATGCTCCTCT
CCTTTACAACAAAGATGACTAAAACTAGAAATAGCTGGTGTTGTTGCAGGGACTCTCATAGAACTAGTAGCTCTAGAAACCCCTTAAAGAAGTT
TGCAGCCTCTGGTCCTCACACACAGTAATTTCTAGACCAGGTAAGCCTACGTGAAGAAGATGAGAGGAGTTCTGAACACTGGTCTCCAACTTCT
AAAACATGCACCCTACATTATAAAATACATTAAGAACATATCCTTTATGTTTATGTATATGCTGGTTTGTATCTTGCATTGACAAAATATATAC
CATTATACATTTGTAGATCCCTCCAATCATGACTGAAAATATTCAGAAAGTGTTTTAAATTATTGGGTAAAGAGAGCTATTGAGGTTAAGAGCA
ATTGCTGCCCTTACAGAGAACCCAGGTTGGGTTCCCAGAATTTACATGGGGTGACTCACAACTGCCTATAATTTTAGCTTCCAAGGATCTGTTG
CCCTCTTCTGGCCTCCTCAGATATCTCTACACTCACAGCATATGGTTACACAAACACACACCCCTATACACATAAATGAATAGTTGCATCTTTC
CTGAATATATACTGACTCTTTATTGGCATTTTAATCAAATCATGAGTGATAACAACTAATTACAGTGTTTACCTTGCACTTGATATGATACATT
ATGTAGATGTGATTTAAAAGATACGAGAAGAATATCTAAACAGTGTGCCATTTTAAATAAGTGATTTTGGCATCTATGGATTTTGGTGTCTTTA
GGGTAGTCCTGGAACCCATGCTTATCCAGAGGAAAATAAGATTAAATATTTGTTATAAACAAAAAGATGAGATTTTAGAAGGTGATATGAAAAT
GTTTAAGCTGACATTCTGTTTTCTTCCCTTATTTATATTGCTTCATCCCTGGTTCCAACATTCAAAACGGAAACAGTGAACATTACTGAGACGA
ACACATATATTTACATTCATACATTTGCTTTCTAGCAAAGAATAACTACTTGGCATTAATGGCTCATGCATAGAAAGACATGAGGCAAGTCAGT
TATGTTCATAAGTAGTCAACTTTCAAGTCTCATAGTCAACTTGAAAGGTCTCATGTCAGTTTGCAAATCTACAAAGTCCTATTAAGGGGGAAAT
TGAGAGGCACATATCCCTGATGCACTGCTAAAAGAACAGGATGCTCACACCATTAGTTCACCTTTAAAAACGTGTGTGTGTGTGTGTGTGTGTG
TGTGTGTGTGTGTATTTGTGCATGTCTGCCTCTGTCCCACCTCCCTCCTCGTAACTCGACTTGCCACATCAGGATGCCTACATCCTAACCCTGA
AACTGGAGTTACAGGCAGTTATGAGCTGACTGCCATGTGAGCTGCCCAACATGGATGCTGAAACACATGCCTATGTCTTCTGTGAGGGCAAACA
GTTTCAACTCCTGAGTCATTTCTCAGCCACTGGTTAGCCCTCTAGACAGTTTTGTTTCCTACACAAAGCTGCAATGCTTTCTCACAATCCTGTA
CCCAGACTCTCATGGTGGTTCATTCCATGATCCAAAAGCTGGTCTACCCTGACAGGAGCAGAGCAATTTCCTCCATCAAGCCTTCGCTCAGGGA
GGAATGACATTTAGTGCCCAGATTGAATGGTCTCAGCTGACAAACAGGCATTTATGTCATCCTGACTGTCAGTCCCCTCTACCACTCTGCACTT
GTGCCATGAAGGCCATTACATCTGATAAACTCTGCCAGCAAAGCAGAGAGGGCACAGCAACCCTGTGAGGGACTCTAATGGAAACCGATCCTCT
CCATGTCCCCACCTCAGCTCCGGACCTGTCCACAGCACTTCAGAGCAGGTGGCTGTCTTAGTCCATTTGAGAAGTCATGGCAGACTGCTGTAGG
CTCGCTGGCTTATAAACAACAGAAACTCAGTCTTCACAGTTCTGGAGGCCAGAATTCCAGTGCCAAAGCACCAGCAGATTTGTCATTGATGGGG
GACTCACTGTTTATTCCTGATTCACAGGAGGTCCCTTCGCACTGTCCTCTCACAGAGTCCCACGCACCAAGCACCTTTCTGACATAGTGTCTGT
GTGTGTGTGTGTGTGTGTGTGTGTGTGTGTGTATCTACGTTTATTCATGAGAGCTCCTCTTTATAGGTCCACCTCCAAATAGCCACACATTGGG
GATTAAGCTTTAATATATGAATTTTACAGGGGAATGGAGGAATATTCAATCTACAGCAGAAATTTTAACTTCCTGGTGTCAAAATATTGTTGCT
ACCCAAAGAAAGTTGTATTTTCAATATCTGTTGCTTTGAAAATTWTTTAGAAGTCAAAAAACACTAGTCATTTAAGAACAATTAAAGTGACCAA
TAATACCTGCATGCAGTTGGGGAATGAGAATAACACATGAGTTCTGTCCATGGCAAATGAATGAGATGTGGTTTCATAAACAGAATATTTGCAC
TCGAAACCAAGTGGGAGAAAAATATCCAGAATACCAAGGAAGAAAACAATATCCATTAATCCCAACCAAGACACAGCCAGAGCAAGATATTATT
TGTGGATATTTTAAAAACCACTGCAAATCAAAGATTATTTTTAAAAATTGCATCTATATTCAATGTATATTAATATTTGATTACCCCTAAATAT
TATTTCACTATTATATATAATAATATTTATAAATATATAAGGCATTATATATACAATATTCTCTTATTCCATAAATAATACAGTGCAACAATTA
TTTCCACTGTTCTACTGCATTAAGCAATAGAAGTAATTTAGAGTGATTTAAATATATAATGCATAAAATATATAGATGCACTATGCCATTCTAT
ATAAGCCACTTGAACAACCAGGGGTTTGGGTATCTCTGTGACTTTCTGAAACCAATACCCTTCAAATTTCAAGGTACAAMAATTAGAAGTTTTT
TTTAAGATTTACTTCTCTTACTTTATCTTATCTACATGGGTGTTTTACCTGCGTATACACATGTGTGCCACGTTCAGTTCCCTGTACTTAAGGA
GGTCAGAAGAGAAGGGTTAGATCCCTGTTCCTGGAGCTACTGGTTGTGAGTCACCGTCTGTCTGTATTGAGAACTGAACCCTGACCCTGTGCAA
GAAGTGCCCTCAACAGGTGAGCCACCATCTCTCCAGCCTGCTTTCTATGAGAAAGTTTGGACTCTGAGGACTTGCTGTCACACCGCACTTTCCC
TTGTCTTTCTTTATTAATAACCATAATGCAAACATGTTGCTTTGAAAGACAGCTGGGAAAGCACACTTTGGAAGAATGTGCCTGGGGACATGGT
CTGTGAGCATCCCCTCTCCTGCCAGCCAGCATCCTGTACCTTGCTGTGGTCTGACTCTTCACACGGGTTCCTTTATGTCATCGGGAGTGGGGGC
TTGCTTCTAGAACTCAGGAGGPAGCAAAGAGGTGGAGTGTCTGCGCACACCACCAGCGAGCCTGTAGACTAGACAGGGGCAGGGGTGGAGTTCT
GGTGAGCACAAAGGCTTCACTTTTATCCAAGAAGACTTTGTGATTTCTGAAAACATGCACCGAGCATTTATTTCATCACTAGAAGGCTACTGAA
ACGGATACTGCACGCCTTTAGCCATAGACCCTCTAGAGCATTAGAAGAGGTCAGCTGATTAGAATACTACAAATCCCAGGTTCCCTGGGAGGAT
CGCCACTTCTGAGAGGAAGTGGTTCAGCCAGGCAAACCCCCAGGGCTGCAGCCAATTGAGAAAGAAAATGAAGGGAAGTTTTTGAAAGAAATGC
CGGGCTCATGTCTGGGGTCTGTCTCCCTCTCTCACTCCTACTTGGCCTCCTGTCTAATTGTTACAGCCTGTCTGAGCTGGCAAATTCCAAAGCA
CATAGTCAATTTACACGTAGCTTAAAAGCTCTAGAGGAAAACACATTTGCAGTCAGAACATTCACTTGCCTACAATTGCTAGGTTAACCTTCTG
GGCTTTCACCCAGTTAACTCGTGTGTTTTCTTAGTTGGGAGAGAGAGAGAACTTGGGGAGATTAAATGAGTGTGAATATAATCCCAGGGACCCT
GCGTTCAAATTTTGACCCTTTCACGTAGTAGGCAAATTATCAATACCTACTGACTAGGGTATCATCTCCTCCCACTGATCCTGTCTAPACACAC
ACATCAAAGAAACGACCTGGAAGTCCATTGCAAAATGCTGCACAGGATGTGTGTGTATAGACAGTGTCACCTGCTACATTTCTCAGCGGACAAT
ATTAAAACTGTAATAGCTAGGAGCCGCTCTGTCCAGGCACTTAATGAAGATTTTTTTTTTATTTGTGGTCAAGAAGGTGTTTTTTTTTTTGTTT
TGTTTTGTGTTTTGTTTTTTTGTTTTTTGTTGTTTTTGTTTTTCAAATGAGTGAAAAGTAGGAGTAGATTGAGTTTTCCAGACGGTATTTATAT
GTCTGCCATCGCCAGCCTCACTTTGCCGTGTGGGTGCTTGTCCTTCACTGGCTCTGTATGGTCACCTCAAGTCAGTAGAGCATGCAAGAAGCTT
TACGGCTGCAAATAAAAGAGAATGGCCTAACGTGAGGCTGAAACGGAACTTTCTCAGAAGTATTGGCTAGATTGTTAAATTGCCTAAGAAATAG
AAGTTGGCAGCTGCAATTCCCTGCCTTGATAAGCTTGTGCCCAACATTCTCACAGACCTACATTTCCTCCGAGGCTTCACGGGAGGTGGGCATA
CCGCTCTCTTGTTTGCCACTGTGGTGACACTGGTTCGCAGCAACTGGTCACTTGTAAATGTGGAGTGAAGCAGAAGAAAGCGTCCACAGTCAGC
AAGACTGAGTTGTGAATCCAGCTGTCCCTCCCTGAGTTATGAATTCTTGTCTCTGAGGTTCACTTCCTCATGCCTAAAGCTAGGACTGTTGTAG
TATCATCCAGGGATATCAAAGAAGTAACGTAAGATCAGGTAATGAAGTGCTTGCTATCTGTTTTAAAAAAAAATACGGAACTTCAAAATATTAT
ATGGATGTCTTAGTCGATTGATACTTCCTTTTTACATGGCTCTGGTCCCCCTGATATAATACAGAGTTAAATTAGGAGCTGAGAAGATGGCTCA
ATGGATAAAGTGCTTGTTGCTCAAACATTAGAACCCGCATCCTAACCCCAAGCTCACACTTAGGAAGCCTAGCTTGATATCTGCATGAGTAACT
GAGCTGAGTCAGGCAGGTCCCTGAAGTTCTCTGGCCAATCAGGGTGCTCCAAGCTCAGTGTAGAGATCTGGCCTCAAAAAAAAAAAAAAAAAAA
AAAACTATACTGTAAGAGAGGAAGACACGAAAACATGACATCAACAGGGAGAGAGAGAGAGAAAGAGACAGAGAGAGAGAGAGAGAGAGAGAGA
GAGAGAGAGAGAGAGAAATGCCCTACTTTATTGTACATAACTATTTTTAGATTTCAATCACTTGGGACAGGAGAGAGATGCTTGGTAGGTAAGA
GACTTGTCAGGGTAAGAGGACCTGAGTTGAAATCTTAGTTCCATACACATCTTTAATCAACATTGTGTGGTAGGAAGTAGAGGGAGACAAGAGT
TTGTATCCCTGTGCCTTGCCAGTTGTGAGTCTAGTTCCAAGTTCAATAAAAGACCTTGTCTCAATGGAGTACGATGAAGGGCAATAGAACAGAA
CACCTGAAACCATTCTCTAGCCCTGAGGATGTGTGCACTAGCCTGCTTCTGCACATACAAGGTTGCATACACCACATTCACTCCGATGCATACA
CAGTCACACACACAAAGACAAAAATTATAAAGCCTTGAGATCATCACAAACTTACAAAACAGCTGGAAAAATATAAGTGAGCGTCTTTTCTTGG
GCTGTTTGAGAGGAGACTCCTGACAAATGCTCCATTAACTCTGGATACCTGAGTGTATATTTCTGTCAAACATCCGTCTTCTCCTAACGAGTCC
ACCACAAGTCAGAGAACCAGAAAATTAACCTGCATAGGTTACTTCCCTGACACTCTCTGGCACCAATCACTTTTCTTTCTGCATGGATATTTTT
TTCCAACTCTGGGAACTTAACCAACCCTGTCATATACTGCTCTACTCTTTATTTTAAGGCAAGACTTCACTAAATTGCCCAGCCTCTCCTTAAA
CTCCCTTTGTAGCCAAAGTAATCCTTATGATCCTCCTGCCTCAGTTTCCATAGTAATGCAAATTAGCAGCTTTGTGCCACCAAGCCAGCCCATT
ATCATGTGTTGTAGGAGAAGAAAGCACCACCCAGAATCATTTGGAATGCCCAGACTTCCCCTGACTTTCAGAATCTTGGGAATTTTAGAACCCC
TTGGGCTGGCCACATTCTAGAATAACTCCCAGGGTTAGGTGCTCGCGTGTCTCTTCATGGTTAGGGCTGCCTTCCCTGTGAGCAGCAATCATAG
AAGTCCTCCCAGGTTCCTCCCCTGGCAGCCTATGTGGAGGTACATGCCCTCAATGTATCTCGATATGAGGGGGTTCACTTAGATGGATGGTTGT
GGAACGCATGATGTTTTTCAGGTTCTTCTAAAAATTTACATTTTTCCTTTTGTAATTAGCATTTTCTGCAGTGTTCCTCATGGAATTATGGAAA
TATCCCCCACTATTAAATTCTAGTCCATGTGTGTGTGTGTGTAAGTATAATAAGTGTTCCTAAAGGTACATCTCTAATTAGTCCTGCTTTCCAA
TTTTATTGAGAGACTTGAATTTGTTCTTCTCATTAGTTTGATGATGATTTTGGCCCAATAGGAAACCATTTAAGTGGGTTTCAACTTTAGCTTA
CGCATGCTAACTTCCTTACTTGTTCCTTCAAAAACACATTTAGGGCTCCAGTCCCTAATTCACGGATTCCTGCATCATTTTAGTGASAAATGAT
TTTTCAAAGCCAATATCTGTGTGCTAGGTGTGCCCATTGTTAGTGGTCTTATCTCTAAGATTTCCCAGAGGATAGCACTAGGCATTATATGCAT
ACATAATAATATGCATATACTATGTGTTTATATACATTTCCATATGTTCATGTATACACATTCATAAGCTAATATGTACTCACTCCATATTCAT
CTCAATCTATATTGAAAATCCTGAGTTCATTCAGATGTGTCCATTTCCAACCAGGGAGCGCACGATTCCTCTACTTCTTCCTTTCCATATTGTA
ATGAAAACCATGTTTTTTGTTTAGACATACAGAGTATCCTTCCTTACCAGGCCCTGCCAAAAACCAGTTTTTCCTCTCTGGACTGGCCGCACCC
ACACGTGACAGGCTCCTTCCCTTGCTTAGGCCAACCCCATGGCCCTTCCCTTCACACAGTGTATGCCCTCTTCAGTTGTTCAAACATAACACCA
CATGCCATCTTCCCTCTCCACAAGGACACAGTCTCCAGCCTGTGGCTGTACTCTGACTCCCATACACAACACACTACCACAGCTTGCCTTGCCC
TGCTGAGCACTACTGCTCACATCGGGCCAACCCAGTGCCACTGCACCTACAGCTTCAGGAGGTGAAGAGATTGTCGAGGCGGGATAGATGTATT
AACCTGTGGCTTTTATCTGACTCTGTCTACACGTACACATATGCACCCATGCATGTGGGTACTTTCTGAGCTAGAAAATATTAGAAATGGGGAG
AACAATAATTATAAATTGGCACCCTTCTAATGATCTTATGTTCCATTATGATATTTTTTTTCTAAATGAC
MOUSE SEQUENCE-mRNA
AAAGTGAAGTCACTTCCCAAAATTAGCTGAAAAAAAGTTTCATCCGGTTAACTGTCTCTTTTTCGATCCGCTACAACAACAAACGTGCACAGGG
GAGCGAGGGCAGGGCGCTCGCAGGGGGCACTCAGAGACSGCCCAGGGCGCCAAAGAGGCCGCGCCGGGCTAATCTGAAGGGGCTACGAGGTCAG
GCTGTAACCGGGTCAATGTGTGGAATATTGGGGGGCTCGGCTGCAGACTTCGCCAAATGGACGGGACTATTAAGCAGGCTCTGTCTGTGGTGAG
TGACGATCAGTCCCTTTTTGATTCAGCATACGGAGCGGCAGCCCATCTCCCCAAGGCAGATATGACTGCTTCGGGGAGTCCTGACTACGGGCAG
CCCCACAAAATCAACCCCCTGCCACCGCAGCAGGAGTGGATCAACCAGCCAGTGAGACTCAATGTCAAGCGGGAGTATGACCACATGAATGGAT
CCAGGGAGTCTCCGGTGGACTGCACTGTCAGCAAATGTAACAAGCTGGTGGGCGGAGGCGAAGCCAACCCCATGAACTATAATAGCTACATGGA
TGAGAAGAACGGCCCCCCTCCTCCCAACATGACCACCAACGAACGGAGAGTCATTGTGCCTGCAGACCCCACACTGTGGACACAGGAGCACGTT
CGACAGTCGCTGGAGTGGGCTATAAAGGAATACGGATTGATGCAGATTGACACTTCCTTCTTCCAGAACATGGATGGCAAGGATTGTGTAAGAA
TGAACAAGGAGGACTTCCTCCGAGCCACCTCCGCCTACAACACAGAAGTGCTGTTGTCGCACCTCAGTTACCTCAGGGAAAGTTCACTGCTGGC
CTATAACACAACCTCCCATACAGACCAGTCCTCACGACTGATGTCAAGGAAGACCCTTCTTATGACTCTGTCAGGAGAGGAGCATGGAACAAAT
AATATGAACTCTGGCCTCAACAAAAGTCCTCTCCTTGGAGGATCACAGACCATGGGCAACAACACTGAGCAGCGGCCCCAGCCAGATCCTTATC
AGATCCTGGGCCCAACCAGCAGCCGCCTAGCAAACCCTGGCAGTGGGCAGATCCAGCTGTCGCAGTTTCTCCTGGAACTACTGTCCGACAGCGC
CAACGCCAGCTGTATCACCTGGGAGGGGACCAACGGGGAGTTCAAAGGCACCGACCCTGATGAGGTGCCCAGGCGCTGGGGAGAGCGGAAGAGC
AAGCCCAACATGAATTATGACAAGCTGAGCCGGGCCCTCCGATACTACTATGACAAAAACATTATGACCAAAGTGCATGGCAAAAGGTATGCCT
ACAAGTTTGACTTCCATGGCATTGCCCAGCCCCTGCAGCCACATCCAACAGACACATCCATGTACGACTATCCCTCTGATATCTCCTACATGCC
TTCCTACCATGCCCATCAACAGAAGGTGAACTTTGTCCCGTCTCACCCATCCTCCATGCCTGTCACCTCCTCCAGCTTCTTTGCAGCAGCATCA
CAATACTGGACCTCCCCCACTGCTGGGATCTATCCAAACCCCAGTGTCCCCCGCCATCCTAACACCCACGTGCCTTCACACTTAGGCAGCTACT
ACTAGAACTAACACCAGTTGGCCTTCTGGCTGAAGTTCCAGCTCTCACTTTACTGGATACTCTGGACTCTAAAAGGCACAGTAGCCTTGAAGAG
ATAAGAAAACTGGATGTTCTTTCTTTTGGATAGAACC
MOUSE SEQUENCE-CODING
ATGGACGGGACTATTAAGGAGGCTCTGTCTGTGGTGAGTGACGATCAGTCCCTTTTTGATTCAGCATACGGAGCGGCAGCCCATCTCCCCAAGG
CAGATATGACTGCTTCGGGGAGTCCTGACTACGGGCAGCCCCACAAAATCAACCCCCTGCCACCGCAGCAGGAGTGGATCAACCAGCCAGTGAG
AGTCAATGTCAAGCGGGAGTATGACCACATGAATGGATCCAGGGAGTCTCCGGTGGACTGCAGTGTCAGCAAATGTAACAAGCTGGTGGGCGGA
GGCGAAGCCAACCCCATGAACTATAATAGCTACATGGATGAGAAGAACGGCCCCCCTCCTCCCAACATGACCACCAACGAACGGAGAGTCATTG
TGCCTGCAGACCCCACACTGTGGACACAGGAGCACGTTCGACAGTGGCTGGAGTGGGCTATAAAGGAATACGGATTGATGGAGATTGACACTTC
CTTCTTCCAGAACATGGATGGCAAGGAATTGTGTAAAATGAACAAGGAGGACTTCCTCCGAGCCACCTCCGCCTACAACACAGAAGTGCTGTTG
TCGCACCTCAGTTACCTCAGGGAAAGTTCACTGCTGGCCTATAACACAACCTCCCATACAGACCAGTCCTCACGACTGAATGTCAAGGAAGACC
CTTCTTATGACTCTGTCAGGAGAGGAGCATGGAACAATAATATGAACTCTGGCCTCAACAAAAGTCCTCTCCTTGGAGGATCACAGACCATGGG
CAAGAACACTGAGCAGCGGCCCCAGCCAGATCCTTATCAGATCCTGGGGCCAACCAGCAGCCGCCTAGCAAACCCTGGGAGTGGGCAGATCCAG
CTGTGGCAGTTTCTCCTGGAACTACTGTCCGACAGCGCCAACGCCAGCTGGATCACCTGGGAGGGGACCAACGGGGAGTTCAAAATGACGGACC
CTGATGAGGTGGCCAGGCGCTGGGGAGAGCGGAAGAGCAAGCCCAACATGAATTATGACAAGCTGAGCCGGGCCCTCCGATACTACTATGACAA
AAACATTATGACCAAAGTGCATGGCAAAAGGTATGCCTACAAGTTTGACTTCCATGGCATTGCCCAGGCCCTGCAGCCACATCCAACAGAGACA
TCCATGTACAAGTATCCCTCTGATATCTCCTACATCCCTTCCTACCATGCCCATCAACAGAAGGTGAACTTTGTCCCGTCTCACCCATCCTCCA
TGCCTGTCACCTCCTCCAGCTTCTTTGGAGCAGCATCACAATACTGGACCTCCCCCACTGCTGGGATCTATCCAAACCCCAGTGTCCCCCGCCA
TCCTAACACCCACGTGCCTTCACACTTAGGCACCTACTACTAG
HUMAN SEQUENCE-GENOMIC
TACAGGGTACAGTCCTGGGAGCACGCAGAGATCCTCTTCTCTGTGCTCACTTAGGCTATACCCTTCATTTGTAAGACACCATTMAATAAAGTAC
TTTCGTCTGCAAATAATTGCAATTGCATCTTCTAGGAATTCTTTCTCTGGGTCATCCACTGCTGAGCCCCCTCCCCAGGAAGTGGAATTGTAAG
TGAAAGGGGAAAGGCACTTGGGAGGGGGAATGATGTTCAACAAGACTCCTTGAAAAGTCAGCCGGGCTCCAGGCACGGTGGCTCACACCTGTAA
TCCCAGCACTTTGGGAGGCCGAGGCAGGTGGGTCACCTGAGGTCAGGAGTTTGAGACCAGCCTGACCAACATGGTCAAACCCTGTCTCTACTAA
TTAGATGGGCGTGGTGGTGGGCGCCTGTAATCCCAGCTACTCAGGAGGCTGAGGCAGGAGAATTACTTGACCCTGGGAGGCAGAGGTTGCGGTA
ACCTCAGATCGCGCCTCTGCACTCCAGCCTGGGCGACAAGAGTGAAACGCTGTCTCAAAAAAAAAAAGAAAGAAAGAAAAGAAAAGTCAGCCGG
ACACTCTGAAGCTCTGTGGCAGATTTGGCTGTGTGCTACGGCCTAAAATTGGAGACGGCAAAGTGGAAACGGTTTAGGGGAGGAGTAGAAGCTT
TGCAGTCAGGCCAGCAGCATGGAGACTCATGAGGTGAGGGGCCAGAGGCAAGTAGCAAACCTCTTTAAGCCTCAATTTTCTTTTCAATAATATG
AGGATCCTAACTCATATTTTAAAAGGTTAAATGAGGTGATGATAGTAAAAGTTCCTTGGAAACTTCAAGTCATCAAATAATACATGTTCTATTT
GATCGTTCTCATTTAAAAATGTGGTCCTTGTTTGGTTTTTGTCTGTTGTTTATTTTGTTTTTGTCGTTTGGAGATAATTCCACTATGTGTCTAT
CAGAGGACTTAGACTTTAATTCTGAGATCGATTCCCCCCTCACTGTGTCAACCTAGAAGGGTTATGCTTTGTGCCTCAGTTTCCCCACCTGGAC
AGTCATGGTGCTGATTCCTGCCAGCCAGTGTCAATAACCACAGTGAAAGGATACTCAGACAGTCACCTCTACAGCCTTTGAATATGGGTTTTCG
GTTGCTGAGTAAAATGCTCGGAGACGTTCCGGTTACTATGGCTACATAAGAAATTACCCCGAAACCTAGTGGCATAAACCAATTATTAGGCTCA
TGAGTTTTGTTATCCTTCACTGAAGGCTCCTTCACGCTCAAGTCTGGAGGTTGACGCTGTGGTTGGCTGTGGCCTCGGTTCCTCTCCTGCGGGC
CTCTCCATACGGTTGCTCAGCTTGGGCTAATCTGAGCTTCCTCACAGCATGGTGGCGATTTCCACGGACTAGAGGCCCAAGAGAGAACAAGAAG
CAGGTGGAAGTCATAATACCTTTATCACTTTGTCCCGGAAGTCACGCAAGGCCATTTCTTGAGGCAATCATAAAGTTCCATCTGAGTTCAAGGG
GGAAGAAAATAAGCTCTGCCTTTPGATGGGAAGAGAGAGGCAAAGATAAGAAATATTCTGATGGTCATTTTGAGAAAATGCGACTGTGCCTCAT
GCAAAGGAGTTTTCAGTTAATGCTTGATGACTGACTAATGTTTCCCACCTTCTTCCTTATCCAACTTTCCATAAAATTTACCTCCTCTTCCCTT
TTCCCTCCCTGCCCTTTACGCAGTGGAAATGAGGAGCAAGAAATGCGCACATGTAGAGAGGAGAGAGAAGCAAAAGGCTTAGCCTTGCTAACAG
CGGCCACCTGCTCTTGGCACTTCAGATGAGACTTGGAGTGACCCCAACCCAGGCTGATCACCCCCACTGCTGCAGAGCCTGCTGGTTTCCCAAA
GCAGAGAACACAGAGGGCTTTGCACCTAAGTATCTTGTTCGCATTTCATATTCTCTCCGACTGGCAAGAGAAGGGACTTCGTGTCATTATGCAA
ATAATGTAATAAAAAGCCAAAACTAGCGTGCTCCCTGTGGTGACCCTGGGTAATGGATATGCCAGAGATTAGCCTTCCAGAGCCCTGTACTGGG
AGCGGCGGAGTGGGCCCACTGAGCAGGGCAGCCACAGGGAACTTGGTCTGGTTGCTTTTGACATTCCTTTCACCAGACACCTCCAGCCAAGCTG
GTCAGCCCCAGACAACTGTCCTTAGGAGATTCAGGGCAGAACAGAGGGAAAGGAATGGGAAGGACACAATGGGACATTGGTGCTTCTCAGGCCC
CATGTCTTGGGTCATTCTGTGTTAGAAATAAAGCTCAGCCTACGTATAAGGAAGAACAGAGGGAAAGGGTCTCCCACTCATGTCCACTTCTGGC
ATTTTTCCTCCTCTAACTCCACTGGACTCATAACTGTGAAATATTTGTTCCTTCCCACACACAGGATAATGGCAGAAATTTCTGTTATGTCTTC
CCGAGTGAAATGGGGAGATACTCAAACCACCCTCTTCTTCTTTCCTTGCCCCTCTGGAGGCTTTTCCATCTCGGCCTTAGCTAGACTGTTTGAA
GATGCAATGGTTTGCTCATCGTTCATATCGGCCAGGCCACTGTGCGGCCATGCTCTGATCACATCCCATCACTCAACTCATGTGCACATGAAAA
GTAACATTTGACTTAATCTAAGGAAAACAAATGTTGGAAGACACATTTGTAAGTATAACAAGAAAAGATGCATATAGGCACACATATGTGACAA
ATTCCAAAATAAGTGAATTTTTAAATTCGTGAATGCACAGATGTACCCACGCCATTCTTCTGATATTCACTGAGTATGTGGTGTACGAAGAAAC
AGTATAAATAGGTGCTAGCCACCTACCATTTTCATTATCTCACGAACAGATGCCTCCTCTCCTCATTCACAGGGCTATTCAGGCTATGTTGAGA
GCCTTATCTATGGACACCAGAGGCCTGGACTCAGAAACCAGAGACAGGCAGAGGACTGAGCCWGCAATGGAATGACACCCATGGTTTTAAAGGA
AGAGTGGACACGCAGATTAGTGAAATCGGCTCTGTACTCTAGGAGCCCATGGGATTTGCATTTCAGGCTCTATCATCACCAAATCTACTTATTA
GTAGTTTAGGCAACGTAATCTTTTAAACAATCAACATTCGGAGAGTAGGTATCGCTCTGGCTTTATCTGAACTCCCTCTTGACCCATCTCCAAC
CCAATGGTTCTCACGCCATTTCTCAGCATTGGCCTTCATTAAAATTTTTTTTAACTTTTTTTTTTTTTTGAGACACTGTCTTACTCTGTTGCCC
AGGATGGATGGTATGATCATGGCTCACTGCAGCCTCCACCTCCTGGGCTCAGGTGATCCTCTCACCTCAGTTTTCCAAGTAGCTAGGACCACAG
ACACACCACCACACCTTTTTTTTATTTTTTATTTTTATTTATTTATTTTTTTGAGACGGAGTCTCGCTCTGTCGCCCAGGCTGAAGTGCAGTGG
TGTGATCTCGCCTCACTGCAAGCTCTGCCTCCCGGGTTCACGCCATTCTCCCACCTCAGCCTCCCAAGTAGCTGGGACTACAGGCACCCCCCAC
CACGCCTGGTTAATTTTTTGTATTTTTTTAGTAGAGACAGGGTTTCACCATGTTAGCCAGGATGGTCTTGATCTCTTGACCTCGTGATCCACCC
ACCTCAGCCTCCCAAAGTGCTGGGATTACAGGCGTGGTTTTTTTATTTTGGTTTTGTTTTTTTTTTTTTGTAGAGAAAGAGTTATGCCACGTTA
CCTAGGCTGGTCTTGAATGCCTGGGCTCAAGCATTCCACCCGGTTCAGCCTCCAAAAGTGCTGGGATTATGGGCATGAGCCATTGTGCCCAGCT
TTTTAAACAATTTTTTTCTTTTTTTTCTTTGGAGACAGAGGCTCACTCTATTGCCCAAGCCAGAGTGCTGTGCCGCTACATCGGCTCACAGCAA
GCTCCGCCTCCTGGGTTCAATCGATTCTCCTGCCTCAGCCTCCTGAGTAGCTGGGATTATAGGCACCCACCACCACACCTGGCTGAGTTTTGTA
TTTTTAGTAGAAACAGGGTTTCATTATGTTGGCCAGGCTGTTCTCAACCTCCTGGCCTCAGATGATCTTCCCGCCTCAGCCTTCCAAAATGCTG
GGATTACAGGCATGAGCCACCATGCCTGGCCACATTTTTAAGTATTTACTTTGTCCCAAGAACTTCAGGTGCTTGATTTAGGTACTTTACTCAT
ATTATCTCATTTTATCTTTATCCAGAATTACCAGATCAGGTACTGATATTATCCCCAACCTACAGATGAGGAAATGAAGTTCTTGGGAATTGTA
CTAGATGCGTAATGAGCAGCAGATGAAGCTTGGATAGAAACTCAAATCTCTCTGACTCAAGATTTATGCTCTAAGTTACAATAAACACCGGAGT
TACACTTAAAGAGCTTATGGTCTAATTGAGAGGACAGAAAATTCACAAGAAAGAACCTGTGAAGAGTAGTGGTTATGTGCTTAGGCTGTAGACC
TGAATTTGGATCCCACATCACACTAACTTTCTGTGTGCCTGAGCAAGTTATTTCACTTTTCTGAATTTCCATTTTCTCATCTGAAAAAATAGAA
TACATCCCTCATAGAATTGCCAGGAGGATTAAATTAGGTTACTCATGTAAACAGTGTTTTGTATTTAATAAGTGCTGTGATTATTACCACTTTT
AGTGCATTACTGGTAGCAGGCCCCAGCTGGATCCAGGACCAGGAGGGGGTCGTGGAGAGCAAGCATTTCTATCGGCAAATGTGAGTGATCTGCT
GTAGCTCACATGCCACCCCAAGCAGCTTGCCTTGCCAGAGGTCTCTGGTAAGGCGCATCATTGGGTACAGAATGTGCTGGAACACTTCCCTGCC
TGCAACAAGGGACCCTATGCCACCCTCTGGATCTAGGACTCAGTGGCTCTGGACCCACAGACCCAGCCTTTCCAGCATTCTGGAAGGAAGAGTG
CTTCTCAGAAGCCAATGACTAAGCCAGCCCAGGGGTTCCTTTTACAGAGACAATTGTTGGGTCAAGAAGGAACCCCACAGCTGAGCCCCACATA
GGGCTAGGAGCCCACGCCTCCTGTCTTTCTCACTTGGAAGTCAATTTTACATAGCATGGTCTAAAGCCTCATGGACCCCACAAGTCTTTTAAAT
AATAACTAACTTCTCTTCCCTTGAATAAAATAATCAGCAACAATAGGAGAAGCAAAGTCATGAGAAACGGAAGGAATGTAGTCCCTCTGAGGAT
GGCTGGTGAATTTGCATGCTGGTGTTCTTCCTACACCCAGCCTATCACCTCAGTTAAGTACTTATGGATGATTTATCCGTGGmGTTCTTAGAAA
CGCTAGCAACAGGCTCATTTCTGTAAAGAGCTTGAAGTACAAGGAAGAGTTTTAAGTTGAAACTTTAAATAGTTTTAAGAGTTTAAGTAAAGAG
CTTTATAAGACACACATGAAGTACCAACAGCTCCTCATGTGTAACAAGCTGCAAATAAGTGAAATTTCTGCCAGCTCAGCAGCTCCTCCCCTGC
AGACAGCAGCAGCCACATCAAGTTGCCCAGACTCATTCCTCTTGTTTGCCTCCTCCCTCCCTACTCTGGTCTTCCCACAGCAATTTGGAGAGGG
CTCCCTACTACCAGTGGCATCATAAAAATGACAGTGTACTTGCCAAGGGCATGGTCTTGGGGTGCTCCTAAAGCTGTTGAACTGCAAAAAGGGG
TGCCGTTTATTTGCAGAAGCCACAAGAATGCTCTCCTCCCCAACTCTGCTCTTTCACTTCGGTCTCAAATCAGCTTGTCTCCGGCCTTATCTCC
CCAAATCTTTTATTCCACTTAGGGGCCCTCACCTCCCTTCCCTTGCCCATATTGGCTTTTCGAATTCAGCCCAGTGAGCCAGACAGCCAACTCT
TCTCCTTCGTCTTCTCTCTCTTCACAGCCCCCATGATCTATCTCTTCTTTTCTCTACATTCTAATGGAAAAAAAGCTTTTTGTCTAGCCATGAT
TGCAAATAGAAGCGGGATGTGTGTGTCTGTGTGTGTGTGTGTGTGTGTGTGTGTGTGCACATATGCATACCCAAAGCAGTATTCTCACGAGCTG
TTAACAGCTCAAAGGAGTCATAGGTCATCATCAGAAAGGTTTCATGGAAACAGGAAGCTCTCAGGAAGATCTCACTTACCTTACACGAATGGCA
AGATAATTGTGCATCAGGGTGAAAGAGTAAAGAAAGGTTACTTGCAGCAAAAGGAACCACAAAATTAAGGCACAGAAGCATACCTGAGTAGGGC
TTGTCTAGTGGAGCAGTGAGAATAGTTAGAATTGGACACAAGTTGCAGCAAGAGGAAGTTGTGCAATGTAAGTGTGGCAGGTGGGACAAAGATG
ACCTTTGGAAGACAAGTGGCTCAGAAGAGATTTGCTGTGGTCCACCTCTAGGGAGCTCTCTCAGGTTTTTGAGCAGATTGTGGCACGATGACAG
CAGTATTCACAACTTGCAAGGGAGCTGGATCCAGCCAGGGAGCGCACAAGGTGTACTGAATGAGGGAAGAGCATGTCAGTGGGTCGTCAGGGAG
GGAAGAGTCTCAGAGAGAAATCCCCAGACTGGACAGAAGTGTGAGGCATTCCCGGTGAACCGGGACGCTCTCCTCCTACAATGTTGCCACTTGA
TTGGAGTTTCCTTGATTGTGAACTACATTCTTAAGGAAACTTCTATTCCAAAATGAGTTCAAACTGGTTTTGTGCCCTTTCTACACCTACACTC
TATCTCATCACTTGTTTTTTTCTAGCTGTGTACCACTCACTTCCTTAAGAAGTCTCTCCTGGGTCTGCCTCCAGAACCGCTGGCTTTGTGTTCT
CTGTCTATTCTCTTTGCTGTCAGTCTTGATTTTGCCCTCTGCATGTCCCCTTGGTCACGACAATCCTGTGTTCCCTGTTGCCTGCTCCACCCTC
CTTAGGATAATGATGGGAGGAACTTTTTGCCCATCTATGGAGCATCACTGTGCCCAGCCCTCCCTCCCGGGCTTCTTTGCCTCTCATATTCTGT
CATCTTCTCCCCCAACCTAGAAGAGGGAAGCGAAGAGATTATTTTACTGGAAAACTCCAACGTTCATGAATGATAAACTAAGAACGTAACCCAT
TCTCTACAGCAGAAGATGCTACCTGATTCCTGAAATGTCATCTGTTTTTGTAGCAGGACTGTGGTACATGTTCTTCACTCTTATCAGCAAGAGA
CACTTAGGGGTCATAGGTGGTAATGTGTCCTCTGCTTCCAGTGCGGAGCTCAAGGAATTTCATGGAGTCAGGGACAGCTGTCTGAAAGGTTGCA
ATGTCACCCCTTTTCAGCTCAGAACCATAATTCCTTGAGCCCAGATAACAGTTCAAAATTTAGGATATGAAGCCAAAGCTGAACTCTAGCTGAA
TCAAGAGTGGATATAATAACCAAGAAACAAATGGTTCTGACAAGCACAGCTTTACTCTGAGCATGACTCAAAGGGCATCTGGTAGAAAATGAGA
AGGATCCCACCAGATGTTAGGGCTAGGGAGATCCCCTCCGTTGGGGGATGTAAGTGTTTTAATATTGTGTGGGCAACTCTGCTTTGAATGTATG
TCTGGAGGCACCTACACCATCCAAAGCCTTCTGGTGCAAAAGCAGGACTCCTAGAGTATATTCCAGTATTTTTTCCCTGATCCCAGCTGAGAAC
AGCCATAGAAGAGCCTCCCTTTTTGAATTATGGCTGTCTGTCCGCTGGGGTGGAGAATGGCCCTGGGAGACAAGAGACAGCCCATGGAGAAGGT
TGTAATTCTACAGGCATATGAAGAGCATTCATTCTGAAGAACACATTCAATTCAGGAATGTGAATTACACAAGGAAGGTAGACAAATCCCAGGT
TTAACAAGTGCTGTTCACCTATCACTACATTTTCAAGTAAAGGTGGTGTATCCATTTGCTAGGGTTTCCATAACAACATACCAGGGCTGAGTGG
CTTAAGCAAAAGAAATTTATTTGCTCAGAGTTCTGGAGACTGGAAGTCCAAGATCAGGGTGCCAGCAGGGTTGGTTTTCCTCAGAGGCCTCTCT
CCTTGGCTTGCCAATGACTGCTGTGTCCTCACATGGCAGCCCCTCTGTCCACACACTCCCCTGGGGGCTCTCTGTCTGTCCAAATTCCCTCTTC
TTATAAGAACACCAGTCATATTGGATTAGAACCCACCCTATCAGCCTCATTTCACCTTACCTCCTTAAAGGTCCTATCTCCAAATACAATCACA
TTCTGAGTACTGCCGGTAAGGGCTTCAGCATATCAGATTTTGTGAGGACACAAGGCAGCCCCTACACGTGGCCTGAAAAGAATCTTTGTTTGAT
GCTAATAATCATGATACCTAACATGTTCTGAGTGTGACTCACTATGTGCCAGAGACTTCTAGTACTTTGCATGTATTATCAC2AACTATTATCA
CAATTATAGATTATATAATTATTGATTATATAGATTATAGATCAATTATAGATGACTACCATCTTCTCTAAGACTTACTTTGGTGTAATTTATG
TGACTGTAGTGAACACTATGCCCTGGTCCCTGACCTTGGATGCCTGTGATTCGATGAGACATGGAATAATGGGAGACTCTTACCCTCATGTTTT
GATTGGTAGCAGATCTCCGGAATGTGCAAAGTTTTTTAGTTGTCGTTGTTGTTGTTGTTTCGAGACTGTCGCCCAGGCTGAATGAATTGGCACG
ATCACGGCTCACTGCAGCCTCTACTTCCGGGGCTCAAGTGATCCTCCCACCTCAGCCTCCCAAGTAGCTGGGCACATGCCACCATATCTAGCTA
ATTTTTTCTTTCTTTCTTTCTTTCTTTCTTTCTTTCTTTCTTTCTTTCTTTCTTTCTTTCTTTCTTTCTCTCTTTCTGTTTTTGTAGAAACACA
GTTTTGCCACATTGCCCAGGCTAGTTTAGAACTCCTGGACTCAATAGATTCTCCCACCTCAGGCTCCCAAAATGCTGGGATTACAGGCATGAGC
CACCATGCCTGGCCAACGCTTCTTTTCTTAATCCATGATCTCCCAAGTCACACCCAAGTCAATCATCCCATAATATGAATCCCAGGACCCGAAC
ACCTCTTTTGGAGGGTCTTGCCCTGACTGGCACTTCACGTGTCTGACGTTCCTCTTTCGTCCTTTGGCTGGCTGAGTCATCAGAACTTGAGATC
TACAGGATGCACCAGCACCTCCTAGAGGGCCCTCAGCAAACCACTGAGCATTTCTTACAAGCTACACCCCCACACCAGATCTTCTCATTTAATC
CACCTATCGACTCTGTGCTAAAACTATTTTTATCCCTGTTACAAATAAGGAAACTGAAATTGGCCTGGCCACACCCATGGGCACCACCCTTCCC
TTGTGTGACCTTGAGCAGCACACCTAGCCTCTCTGGGTCGATCTCACTGGCCTTGCCTGGAAAATAGACCCTGGATCTCATCTCCCCTTCATTG
CTGATGACTCCACTCAGCTCGTAAGAGGCATTTCTGCTTATGCCTGGAAAAATCTAGCATTCACACACCACCACAATACTATATTCCATGCCAA
GTCACCACTATTCTTGGCCTCCCTATCCCCACCCTCCTTTTTGGGGGTCCCAGACTACAGAACCAATAAACAAACCCACTTTCATGATTCCTCC
ATCCCCACTCCACTGCCCAAAGTCCCTCCTCACCCAGGTAACCTGCACACCCATCTGGTTTGCATAAGGGCAAAGACCTGCCCCCTGGAGTGTG
AGTCAAACAGGAACTTCCACACAAGCTGTCCTGGCGCAGCCCTGGGCCACCTTGCCAGCTCAAGCAAGAGTGTCACTTGCTTGGCTGAGAGAGT
GACACTGGGCTTTCTGTCTCTTCTCTGGCCCTGCAGGAGGCTCTGTCGGTGGTGAGCGACGACCAGTCCCTCTTTGACTCAGCGTACGGAGCGG
CAGCCCATCTCCCCAAGGCCGACATGACTGCCTCGGGGAGTCCTGACTACGGGCAGCCCCACAAGATCAACCCCCTCCCACCACAGCAGGAGTG
GATCAATCAGCCAGTGAGGGTCAACGTCAAGCGGGAGTATGACCACATGAATGGATCCAGGTAAGCTCACCAGGCCTGTGCAGGATTGGGGGAA
GGCACAAGTCGCCAGATCTGCAGCAAGTGGCTTCTGGCACTTAAGGTTTTTGCACCAGATGGGGCTCTAGACCTGGGCAAGGAGAGCCTGTGTC
AGTCCCTAGAGAGCTAAGGCAGAGAGGGCCACAGACTGATGATGGGGTGCAGTAGGGCTAGGAGGCCCCCCTGGCTCTCTGCGGCCCACGAGAC
CCTCAGAGGCCAGGTAGGAGTCAGGCCCCAAGCGCCTATGCAGACACTCGTGGAACACTTATTTCTTGTTAATCCCAGGCCCAGGTCCAGTCCT
GTTGGGGACAGCTTAGCAATAAGCTACTCTTCCCTGTCCTGTTGAAGATACTTAACCCCTCTGGGCAGGTCTCAGCAGAGCAAAGGTGACCAAA
CCATTCTTTAGTAAGAGTGGAGAAGGGATGAGGAAGGTGTCTACCTTCCTGTGGGGGCTCACTGTGTCTGGGAGGCGGCAGGCACCTGGAAGGC
GTGCATGAAGTGGGTACAATACTGGGTCAGTTCGGGCTTTGCTTGTAGATGGAGACAGGAGGGTCCAGGGAGAGGAATGAACTCAGGGTCATGG
AAGAGGACAGTGGACAGGCGGCCTCAGTTTTGCGTCCTGCCTCTGCCCCTTGGGCAAGTCATCGAAACCCCTCAGAGCACCAGCTTTCTCCCTT
CTCAAACAGGACTGGGATATTCACTTCCTTATGGTCCTTACTCTAGCGTTACTCTGATGCCTCAGGGGAAGGACCTGGGATGGTGCCTGAGACG
CGTTAGGAAAGTGCTCTATAGTGACAGTGATTGTTATGAGCAAAGACTTGAAAGACAGAGAACATCAGTCTTATTCACAAAATATCAGATAATC
TACTGGGCCAAAGGCACAAAGAGATGAAAATAAAAAGCAACAGAGGGCCCTGAATGGCAGGCTGAAAACATGTGCCATTTATTCCACAGCCAGA
GGCTGACTTTGAGCACCGATGTGACATTTAACCCAGAGGCTGACTTTGAGCACAGACGTGACATTTAACCTCATCAGAGCAGTGACTTAAGACT
AGCGAGCTGACAGTCCTTGCGCACAGACGGTAGGATGGGAGAGTCAGAAAGACAAGTGAAGAAGTTAGTAAAATATCCCAGATAGGAAATAATG
AAGTTCTGAACTAAGTGATGATAGTGATTTTGGAAAGTGAAAAGGACAGATACATAAGGGCGATAGCAAAGAGATGATTAACAGGGCTTAGGTG
AGTAAAGTGTGAATGAAGGGGGTGCATGACAGGAAAGAATCAAAGGCGACTAAAGCCTGCACATTTTAAAGGCTGGTGCTGTGAGCAGCCGCAG
AAAGGTGGCACAGGAAATGCTTTAGGATGGGATGTCAGTGTTCTCTTTGAGATTCCTTGTGAGCAATGGGTAGAACATCCAGATAAAAATGTCT
ACTAGGCTTTGGGAACTGAAGCTCAGATAATCTTTCAGGCCTGGAATTTACGTTTGGGCATCATCTCTAAGGAGTCTGAACTGTGAAAGTGGAT
GAGGTCACCGAGGAAACTAGTCTAGATTGAAGAGAGAGAAGGGAATGAGCATGGTTGGGAGGGAGAACTCCTCCAGAGCAGCTCATGGCCTCAG
ACCTGCCTGTCGGAACTCCCAGGCCTACCACATTTCAACCCTACTAACCAAAACCAGTGACTGGGATCTCAGCCACCTTTCCAGATCCCAGGAT
TTCAGGAAGGCTCAGAGGTTCTGAGCCTGCAGCCCCTCAGATGAATCTCTCTCCAGATGTAATTCTGATGGTGCCGGAGCTCCTAACAGTCGAA
GGACATGGTGTGTTGAACGTTTTCCCATATTATCTTACCACTTGTTCCTCACCACTATACTAAGAAGTAGTTACTGTAACTTCACTGTTACACA
CGAAGGCAAGCAGGGCTTTCAGGGGACAGTGCTTTGTCTAAGGATTTACGAAAAGCAAAGTTGGAGCCCGAACCCAGGCGTTTGGTTCCAGCCC
CACGTGCCATCTGTTAGGCACCTTTCTGCTCCCTCTCCTTGTGTGTCATGCTTTTGCACCACCACCTCATTCCTGCCAATGCCCACCTGGTGAG
GAGGCCTATTCTACGATGCCTCCTAATGGGGCCCTTCTCCAAGGAGCTCCCAATGCTTGTTCAAAGATCCAGATCCCACTGGTCGACATTCCTT
TTTTTTTTTTTTTTTTTTTTGAGATGGAGTCTCACTCTGTTGCCCAGGCTGGAGTGCAGTGGTACAGTCTTAACTCACTACAACCTCTGCCTCC
CAGGTTCAAGCAATTCTCTTGCCTCAGCCTCCTGAGTATCTGGGATTACAAGCACATGGCACCACACCTGGCAAGTTTTTGTATTTTTAGTAGA
GACGGGCTTTTACTATGTTGGCCCGGCTGGTCTTCAACTCCTGACCTCATGATCCGCCTGCCTCGGCCTCCCAAAGTGCTGGGATTACAGGCGT
GAGCCACCGCACCCAGCTTTGTTGGAGATTCTCTGGCCTGTTGTAACCTGAATCTGCCTACTCCTTACCTCCACTGCCCCTGCCTGGTGGCTGC
CAGCAGCACCTCTCCTAGGCTTCTGCGGGGGCATCCTGCTCTCCCCACTTCCTCCTGCCTTCCTGAAGTCTATTCTCCGGACAGCAAGCAGCCA
GAGCCATGCTTTCCTCGTTTGTAATCCTCTTGCTCAAAACTCTTCAATGGCTTATGACAAAATGCAAAGCCCTTAGTGTGGCCCCACCATCCCT
CTGACATCCTCTCTACTGCCCCCTCCCTCCCACTTCTTCCCTCCGCGCCAGCCTCATAAGACTCTAGCTGCTGCTCCTAGACCCCAAGGCACCC
TCGTGCCTCAGAATCTCTTCCCCTGAGCTCCTTCATTCTGGAACATTCCTACTCCATCTAGTCATATGACTCTCTCCCTCACGAGTTTTAAGTT
TCTCTACCTGAACCTCACCTTTTCAGAGGCCTCCCCTGGCCACCTGATCGAAACCAGCGCCTCCTCACTCTTTACCCTGCTTAGAGTGTCTGTC
CTAGTTCTTATCACCTCCCTCACTGACACGTGGGCAGGGCCTGAAACGGTAGGTGCTCAATAAATTGTCGTCAAATAAATAACTGAGAACACAT
GGCTGAGCAGACTCAAAAGAGAAGGTGCTTGTTTAAAGTTAGGACGCAGATCCAGGTCTGCTGACTCAGCCCCTTCAATGCTGAGCCTGAGTTG
ATGCATGGGCTTCTGAGTTCCATCTTCATGCTCAATGGAGACCCATGCGTATTTGTGTCTCTGCTGGGAAGGAAGTATGCTGCTCACCACTCCC
TCCCCTGCTCTCAGAGAATCCTCAAGCACTTCAGGATACTGATCCTGTGCAAAGAGGGTCCCCCAAGGAAATTGATTTTTCAGAACCCACTTGC
CAGCTCATACCTGGCTCCAGTCACACCAAAGCCAGCTGGACAGGGCCTGTGTGAATAATTCCAGTGAACACACAGGATATTTACCTGGCTTTCG
CTACACAGAGCAGCAACCTCTGCTTTTGAGTTGCTTGTCTTGACCCATACCATCAGCGGGTAACAAAGTAGGTTCAGAAAGACTGATATCTTCT
CACCCACCCTGGGCAAGCAGAGACAATCCTGTGGACTCTCAGAGCCAAGCAGCTTAGGCACACCTATCGCCAGCACCCCACTAATATTCCCTAA
GGGCTCACATGTAAGCTGGACTAGTGGCTAGCAGGAAGCAAGGAGTTTTAGAAAAATGTCATTGCTTACAGTGGAACCATGTCAGACCCTATCA
TTTAATTATGCCAAAAGCAATGACAGAATGAAATAAATGCCAGGCTCTTTAAATCTCTCATCCTGAACACTGTGCTTCTCCTATCCTCTGAGTA
TTATTGATAATGACAGTTCGTTACTTGCACATGATTTGATTGTTTGCAGTGGTCTAATTCGCCATAACACAAAAAGATAGGAGAGGATGTTATA
ATGATTCTGAACTTCAGATGAGAAAACCAAGGCAGGGTATAAAATTTGAGACTGCTTGGCTCATGGGCTAGACCTGTGCTGTTGATTGCAGCAG
CCACTAGCCACAGGTTGCTACTGAGCACTTGAAATACGGTTAGTTGAAAATTGAAATGTGCTCTAAATATAAACCACATACTGGATTTAGAAGA
CTTACTATGGAAAAAGTGTAAAATATCTCATTGATTATTTATTATGCTGAAATGATCTTCTTTTGGCTATATTGAGTTAACATATTAATATACT
TTAATATATTATTAAAATTCATTTCATCGTTTGTTTTTCTTTTAAAAATCTGGCTACTAGTAAATTTAAAATTATGTAGCTCGGCCAGGTGTGG
TGGCTCATGCCTGTAATCCCAGCACTTTGGGAGGCCAAGGCGGCGGATCACCTGAGGTCGGGAGTTCGAGACCAGCCTGCCCAGACATGGTGAA
CCCTGTCTCTGCTGAAAATACAAAATTAGCCTGGCATGGTGGCGCATGCCTGTAATCCCACCTACTCAGGAGGCTGAGGCAGGAGAATTGCTTG
AATCTGGGAGACGGAGGTTGCTGTGAGCCGAGATCATGCCACTGGACTCCGGTCTGGGCAACAAAAGTGAAACACCATCTCAAAAAAAAAAAAA
ATTATGTAGTTCGCACATGTGGTTCCCATTCTACTTCTATTGCACAGTGCTGGGCCATGGTGGTCTGGACTGTTCTGTGATTCACAGTGCATTT
TTTCTGCATTCCTTGACAGCATGGGCTTCTGAGTTATGTCTTCATGCTCACTGGAGACCCATTGGCATTTGTGTCTCTGGTGGGAAGCAGGTGT
GCGGTGCACCTGAAGCTACCATGCCCACTTGCTTTTCACCCCAGTAGCAAACCCTCTGGCCCAGGGGCAAAGGGTCAAGTGCAGAGACTGAGGT
CTGAGGATCTCAGAGACAGAGTGGCTTGCCCATGTTGAGGGAGTTTTGTGCTCTCTTTCTTCATATCTTCCTGTTGTGGTTGACTCTGTTAGGC
TGGGTTGCCTTGAAACAGCATGGCCCATAGGTTCTGTGTCGCATGTAGACTTCAGCAACTTGATCCAAACTGTGGTCTGCAAATAACTCTCAGC
TGCATCCTGTCTCAACCACAGGGATAGGGGAAAACATCCCATGAAGAAAAGAATATCAGGTTATAGAGGGAAATGCAAGACTTGCCTTGTGAGC
CTGGTATTCAGGACCAGAGTTCAGAGCTAACCTTCCTTGAAATTCCCTGGCCTGGCCCCCAGGGTCTGGATGCCCTGGACACCTAGGGACTCAC
ATTCCACACAGAGAAGGATCAGCTCCTGTTCCCTGGAACTTGTGCCTCTCTGGCTGCGAGGCCCCTTTCTAAACTCTTGTGTTCTCAACCTTGC
AGTATGAGACATGTCACACATAACCTGGGTGGGGAGGATACACAGGATCCTGGAAAAGTTAGCAGCATCTACTGTGTATGGGGATGAAAACAGC
TAGTGGATTAGGGCGGTGGCTTTATCATAAGTGATGCTTTTTCTTTTCTTGGAATTTGCTTTTAAGTTATTGCATCTCCTTTTCAAAAACAAAA
AAGTCACATCTATGTCCTACAGACTTCCTAAACGCAATACAGATTGTTTAATTTTTGCACAGGGTGCATACTACGCAGGAGTTACACGCCGCGG
GTCCTAATGCAGCCATGTGACTTAGGCAGCCCGCGAGGCCTCACTTTGGCCACGTGAGCATCAGGTATCCTTTGACAGTGAGCCTGGGCTCAGA
TGTGCGGGTCTGATGATATACCCAAGTTCAGTTGCTCTGAGTTGACTAGGAAGGTAGAGAAGATCTTTGTACAGCCTAAAGAGGTAATCTTCAG
GAGAAGTCTTTGAAATCTCCCTTCATCCAAACACCCAAGATCTTACACCTTGTTTTAAATGGCCTAACACATTGCAAAGCTTTTTTTGGAAACC
AGTAGCAATGTTCACAGAACACACACAAACAGAACTCGAGGAGTCAAGAGCTATGTCCAGTGGTGGAAATGAAGGACAAGGGCTACCCTACCCT
CGGCAGCTCTGCTGTGGCCTCTGTCGCCCTGAGATGGAGATGTGCTCAGTTATTACACCCTTAGGGCCACCGCCACCTCCCTACTCTATTGTGC
CAGCCCCAGGCTCTCTTGTCACCTGTCCCAGTGCTATCAGCTGCCAGTGGGCCCCAGCCCCATGCTAGCTGTAAGTGCAGGTCTTAATACAAAT
TAGCAGCAGTGCAGGCAAGCTGAATGGCAAAACCTCGTCCCGCACTCCCCCTCCCTCTTGTTTTCATCAAGCCTTCTTCACAGGCGCCAGCTGC
CTCATTAAAGAGCAGCCTTTTATGCTGGGCTTCACCTGTACCCACCCTGAGCCGCAGCCGTGGAGCCCCATGGCCGCACGCAGGGCTTGCCCTG
GCTGGAGGAGGCACGGTGCTTGGGAGCTGCAAGATTGGAGGGAGGACTGGCAGGCGAGCGGGCGAGGTATGGCTTTCCTTCTTTCTCTCCCTGA
GCAGTGGGAGGAGGGCCAGCTGGGCAAGTGTGGGGAACCAGGATGGTGCCAGCCCTTCTCCCTAAGGACACCCGCCTGCAGGTCCTAGTGCAGA
AGCCTGGGACCGAGGACAGGGCCATTTTCCAGAGGAGAAACACCCTGGCGGGCGAGCGGCCAAACCGATCACCTGTCCAGCTGGAGAGCAGGTG
TGTGCACAGGGCCGGGGGAGGGAGAGAGGAGGAGGGAAGCGGAACTGGGAGACTTCTATGGGTCAGTGAATGAGGAGGTTCTGCTCAGGGAATT
GAGTGGCAGCCCCCACCCCACAAGAGAGCCCCCTCCTCCTCTGCGTCCACTTTCCCTTCGTCTAGGTGCCCATGGCTCCTCTCCTTACAAGGT
CACGTGTTAGGCAGGTTTGGGGAAATGTTTCTTTCAGCTTTTCACATTTATTTTCCTCCTACCCTGGCACCTGGGACCAGCCCTGCCACCTGTG
CCTACCGTACATGGCCATAACCATAAGGCTCACTCCATTTCAGTCAGTCCTGAGGTCCTCACTTCTCTGCACAGCGATCAGGTCAGTTTTTAAC
TCAGAAATCCCCAGGGGCCCGGCTGATGTGGAAAAGAATTTTTCCAAATATCGTATCCCCACCGTCCACAAACGGGAGGAGCCCGGGGAGTGTG
CCCTCCTGTCCCACCATCCTGCCCCCTGCTGTAGTTCTCCTTTCCGACAGACCCCTGCCCATACCCTATGCCCTCTTGCTCTGGGAATCCTCAA
AGGGAAGAGCGTGCAGTGTGGAGCCCACCTCTCCCACAGAGGCCGCCTGCCAATGAAAACCTTGCTCACTTGGTTGCAGGCCAACTTAATCCTT
CAAACTTCTGCAGCACTPGGACTGTTTCACAACTGACAAGGATGCAGGGTAAGGCTGCCAGACAATGTGCGTGAGTGTGTGTGTATGCGCGCGT
GTGTGTAATGTGTATGCGTGTGTGTGACTGTGTTTGCATGTGTGTGCATGTGTTGAATGTGTGTGCAGATGAATGTGTACGTCCATGTGTGTGC
ATGTTCTCTGTGTTGAAATGTGTGCTTCCATGTGGGTGAAGTATGTGTGTTCCATGTTGGAATGTGTACAGTGCGTGTCTGTGCATGTGTGTTG
AAGTGTGTCCATGCATGGGAGTAAGCATATGTGTGCACGTGTTCTCTGTTGAAGTATGTGCATGCATGTGGGTAAAGTATTGTGCACATGTGTT
CTGTGTTGGACTGTGTGTGCACATGTGCATGCATGTGTGTCAGTGTGTGCCTGAAGTTAGCCCCAGGGCGGGTGAGCAAACCGTGTGCACTCGG
GCCAGAGCTCAGCTCAGATGCAGGCACCAGGGTCTGACTGGGACTGCTTCATCTCAGGCCAGGTCCTTCCGCAGCAGAAGCCAAAGGGCCAAAC
GCTGGTGAAAAGTCCTGATGTGCTACTGCCTCCCCTCCCCTCCCCTGCCATGGGGACGTTAAACCTTCAGCAATCTGGAACTCTGACGGCTGGT
TTTTGGTTCTCTGCCTGGAAAAACAGTAGGCGAATAGTAAAACCTTAAATTCTAACATGTGTGAGTTTACATCTTATTTTCATTTTTCAGCCCA
TGTTTCCCTCTGTAAGACATCAGGAAAAATTACCGTCTGACTTCTAATATCTCAGTGGAGCATTTATCTCCCAGCTATGATAAGTTGATCTTCA
ATATTTTTTTCCTTTCAGATTAAGAAAACAAATTCATTTAATTTTCTTTATTGAATGTGCTCAGGCCTCCCCGCCCTGCCTGGGTCTCAGAAGC
CCTGGGCTGATCTCCCTTCTCCTGGGTTAGTGACTGCCGTCCCTGCTTGTGGGATAAGTGGGTCCCTATCTGGTACGGCCCTGCTCCTCTAGGC
CTCGCTGTGGTCCGTTCCTGCGCCTGGCAGGGGATGGAGGACAGTGGCCAGGCCTTTGGAACGTCCGGAGAGGTTATGCGGCCTAGAGTTGGAA
GGCTGTAATCTGAACCTTATCTGAACTCCTTGGTCTGAAGGCCTGGTTTGGAGCTCTTGAGAGAACTGGCTGTCTCCCAGGCTGCCAGAACCGA
TGCTTCCTGCTCAGTATAATGGCGGCTACAGCTTCTCCTTGGCCTTCCCATCGTCTCCTTTGGTCCTGCTGCTGCTGGAGTGGCTGAGCAGCGG
GCACTGGGTGTGACAGGGTGGCACCTCCCCCAAGTATGCTGGAGGTGCGTGATGCTGGGCTGGAGGGCATGCAGAGTGTGTTTGCCAGCCAGCT
TCAGGGCGCCAGGTGCCCATGGGTGCGACTTCACCCCTCCAGACAGCTGCCCATCACCAGTGCTGCAAAATACCAGGTGCTGATCAAGTTCTGT
AGTTCTAGGCTAAGTTGGATGATCTGCTTTCTCTCTTGTGAATGTCCATACTGCTTCCACAGTGAGCTTGGGGCCAGGGATGCAAAATGCCACA
GGGCCACCACCTCTCCATCAGGTGGAAGTTCCTGGACTCCATTCCTTCTCCAAGTAGCAACCAGCTCTTTTGCATGTGTGCTTGATAGTGAAGC
TTAACACAGAAGACCTGAGTCCTGTTTCCACCTTGACTAGCTGTGTGACTGAGCCAGGCAGCCCCTGTCTCGATTTCATTAACCATGAAAAGGA
GATAGTAACCCTTGCTGTGCCTATCCTGGGGGCTTATTGGGAAATTCAAATACAACAGTGCATAAGAAATGCATTAAACTTATATATGTGCAAT
AGAAATAACTCACTGAGAAAGCATTTGCTGCTCACCTCCTGTAAGAATACTAGGGTTAAACAAAGAAAGTGATGACTATCCTCTCAAGAACTCA
GCAGTCAGGGGGCGCAGGCCAAGCTGAGAATCAGACAGTGTGACAAGTCCTTATAACCAAGGCGGGCACCAAGTGCTGGGGGACTGTGGGGAAG
GCGTGTCTGGGAGCATGGACTAGGTTGCCTGGCAGTGAGGCTAAAAATGTAAGCTGCAGTCAGACAGGGAGGGCTGCGATGCCACACAAAGAAG
CTCAGACCTTGGTCTGAGGGCACTGAGGCGCTGCCAGCAGTTTTTACGTACTGTATTATAGGATTGGGCTGGAATTTGTAAGAAAACTCTGGTG
GCAGCATGGAGGATAGAAGAGCGCATCATCATCATCATGACACCAGTGTGGCCGGGCAGTATGGCTCCATGCTCATCATCGGAGCCCTGGGCTC
ATGATTCAGAGGCTGAGGCAAGCTGCCCTGTCAGTGCTGACCGCCTCTAAGCTTTGTCTCTTCTCACTTTAGGGAGTCTCCGGTGGACTGCAGC
GTTAGCAAATGCAGCAAGCTGGTGGGCGGAGGCGAGTCCAACCCCATGAACTACAACAGCTATATGGACGAGAAGAATGGCCCCCCTCCTCCCA
ACATGACCACCAACGAGAGGAGAGTCATCGTCCCCGCAGGTAATTCGAGAACCAGGCTGCCTGGGCGCCATTCACTTCCCCACTCTCTGGGGGG
CAGGAAGCATCTAAACCTTTATCTCATACTCTATTCCCTGTGGAATTGCAATGGAGAGAAGCTGCACGCCAGCCGGGAGTGGTGCCTCGCAACC
TGTAATCCCAGCACTTTGAGAGGCCAAGACGGGTGAATCACTTGTCAGGAGTTCAAGACCAGCCTAGCCAGACGTGGTGAACCTCATCTCTACT
AAAAAAAAAAAAAATACAAAAATTAGCCAGGTGTGGTAGTGGACGCTTATATTCCCAGCTACTCAGGGGGCTGAAGCAGGAGACTCGCTTTAAC
CGGGAAGGCGGAGGTTGCAGGGAGCCAAGATCACACCACTGCACTCCAGCCTGGGTGATGGAATGAGACTCTGTCTCAAAAAAAAAAAAAAAAA
AAAAGCTGCACATTTTACCTGACTTCCTAGCTGCCCTTTAAGGCAGTATCCCAGATGTCACCTGATGTGTCGGTGCTAGAAAGCCCCACCCTTA
ACAGACCACATCACAGACTCTACATCAATGGCCTGGTCCTCAAGGACGGGCTGCTCAGAGAGCAGAGGCAAGGCCGCCTCCACCCAAAAGGAAT
CCCGTGAGCTCTGGATGTGGGCATCCCTGCCTGTGAGGAGGAGGTCAGCTGCAGAGGCCAGTCCTTCCCTCCAGGCTCGTACTTCCCGGAATCT
ACCAGGTTCCACCTTTCCAAAATTACAGCCAGCTCTTTCAGTTTTAAAATAGAAAGAACTATATTTAACTAATGGAGTGCCTTAACATGTGTGC
CACAAGTTATGTCATTAGGTAAAAATAGCCTCTTTTTCCTAGGTGTTGGTGCTGTTGGTCCCAGAAAATAAAATCATCGTTCAGTCCTTCAGTC
CATTGGCTGGACACTCCACCCCAGCTAGCACCAGCAAGCTCAGCTGCCTGCACAAGAAGGGTGTTTACAAACTGCCACGTGTAGAACTGGTGCC
AGGTAGAAGGGGCAAGGATGTCCAAATCTTAAGCAAAGTGATTACAGGGCAGGGATTAAACCTGATCTTACCAAAATAACTTTACCAGAGAGGC
AAAACTCCTTCCATTTGTAAAAGCATAAATGTAGTTGCCTCTGAAGTCAAAAGGTTAGACATCTCTTTGAGGCAGTCCGAAGGCATCTGCTTGT
AGGTCTAAAGGCAGAAATGTTAAGGGCTGCCTCCTGCCTTGGCCCGTGGTGTTTGTTTTCCCTGGGAAGCTGCATCTCACCATATCCCTGGATT
ACAGCTGCCTCCCTGTGCTCTGGGCCAAATTCAAGAAGGTGTGAGCAAGAGTGAGCAGCAGACGGGTGGGGCTTGTAGAAAATTTCTCTGTACC
CTTTAGGACTTAGAAATGACTCCTAAAGCAATTCTAAGTTTGCTCTTCCACAAGCATCTGACCTAACAAAGGAAGAGGAAGAATCACTTCCATT
TATTGAGTGCTTTCTAGGTGCCAGGCACCACGTAAATTGCTTTATCTCCATGGCATTACTTAAAACACCTGTCACAGGAGACCAGCTAGTAATG
TTTAGCCCAGGTGTATCGTCTCCCTCCTCACTGAAAGCAGACAATGAAACTGTATTAGAACTGACAGGTGGGAATCTCTTTGTACAGCCCAAAG
CAAAGCTGAGCCTCACTGGGTCTTCACTCAAATGGTTTCAAACTCCCCTGCTTCAGTCAAACAGCTCCCAGCCTGGGAGGGAAAGCTTGCGGGA
CTGAAGGGTTTGGGGATCTGAGATTGTCTTTGAACCAACCTTTGGCACCATCTCATTTTGGTCGTCATTTTGGGGAAGGAATCTTGGAAATGGG
CAGAAGTGGCCAGACTGCTATGTCGACCCTCCAAACTTTCTTTGATCAGTGTTGAAATGCTTTCAAAAGGAATAGCAAAACATCAGGCTTGGCT
GAGCCAAGTTACCTATAGCGCTGGTGGTGAAAATAAAACAAATTCATGTCAAATGATTTTCAAAGCACTTTCAGGCTATGGATTTACTTCATTA
TTTTAGCTCCGGCTAGATCTTTTAGGTCCTCACACATTTTGGCTTCCTCCAGGCCAGGCTGTGGATATGAGGGGAAGTCTCATTTGGGCAAGTC
CACAAATCAAGGCAATAAAATATTAGTGGAATAACCGATTGAATTTATGATGGGATTTAAAGACAAGTATAAATGTGGGCCCTAGTTAATCACT
AGCTCCATGTTCGATGGGAAGAAACTAGGACTAAATCATCCAGGAAATGCATTTGGAAGAGTTTAGCATGATCCAGAGTTGAAGATTTATAAAT
TCATTGTGCTGTAGAGGGAGAGTAATAATATTGTTGCAGAGCCCAAAGCAAGGGAGGAAATAATAAAATAAATAAAATATATGGTGTAAAATTG
AATTTGAGGCCTTGGATACTAAAAGTGAGGAACTGGGAGAAGTGAAGAATGAAATAATATGCTACATAATCCAACTCTCCCGAGGATATGTTTC
TCTAGAATTCTGAAAAGGTGCAAAATTATTTCCATTTGCCAATGATCCATGCCTAATTGCTTCTGGAGAGTAAAAGAGCAAGAAATGTATCTGA
GAGAGAGAGAGGAAGACATACTTGTACAATGACACTTTAGAGGAGCTCCGCTTTCTGGCTTCAGAGCAGTGTAACTCACAGGTGAGGTAAAGCA
GGATGGTGAGAAGAGGAAAGGGGCTTTGGGGGAACCCACCTCTTCAGCTTAGTGAAGTCCTATAATGGTTTCTATTGGAACCCCAGCCCAGATA
CTTACCAGCGATGGGGCTTTGGGCAAAGGACTTAACCTTTTAAGACTCCATTTTGCATCTGTAAAATGGGGATATTAAAAGATTTCCTTGGTAA
GTAAAAATAGGGCTCGCGGAGCAGGTGGCACATAGCACCCACCATCTGAGCCGTCACTGCTCACAGGGCATTAGGTTGTTGGAATGACATTCAG
AATTTGAGGCCTTGGATACTAAAAGTGAGGAACTGGGAGAAGTGAAGAATGAAATAATATGCTACATAATCCAACTCTCCCGAGGATATGTTTC
TCTAGAATTCTGAAAAGGTGCAAAATTATTTCCATTTGCCAATGATCCATGCCTAATTGCTTCTGGAGAGTAAAAGAGCAAGAAATGTATCTGA
GAGAGAGAGAGGAAGACATACTTGTACAATGACACTTTAGAGGAGCTCCGCTTTCTGGCTTCAGAGCAGTGTAACTCACAGGTGAGGTAAAGCA
GGATGGTGAGAAGAGGAAAGGGGCTTTGGGGGAACCCACCTCTTCAGCTTAGTGAAGTCCTATAATGGTTTCTATTGGAACCCCAGCCCAGATA
CTTACCAGCGATGGGGCTTTGGGCAAAGGACTTAACCTTTTAAGACTCCATTTTGCATCTGTAAAATGGGGATATTAAAAGATTTCCTTGGTAA
GTAAAAATAGGGCTCGCGGAGCAGGTGGCACATAGCACCCACCATCTGAGCCGTCACTGCTCACAGGGCATTAGGTTGTTGGAATGACATTCAG
CAGCAGGCTGTGACCTGTGAACCCAAACTCTCCTAACTCCTCCCCTTTGCCAGCTCCTCCACCTTCAGCACAACACAGCAGCCACTCTCGTGCA
TGGGCAGGGCCTGGGCACTTTACAAAAGCAGACTACTCGGGCCCTGCCACCCAGATTCTCACACATAAAACCAAGGTGGAATGGAGAGTCCAAG
GACAGTTGGTGGATGCTTGTGTAAAAGTGCTGGGAAACTTGCCTCTGGCTGCCCAGCTGTGAAGCGTGAGCCTTGCTTCTTGCCCCACATCCAC
TCCTAGTCATTCAATCCTGGTTCCCTGCTTTGCCAGGAGCCTTCATAGACGCTGTAGAATGAGAGGGTGAGTTGCTTGAGGGGTAGCACAGATA
TTCCTGCCTAATAAATATGTTAAATCAAACAGGCTCATTAGAAGCCGGAGGCTAATGGCCGACTTACAATAAAATGCCGAGTCTCTCCTCCCCC
AGGAAGTCTTCCTTGACTCTCCAAATTTTCACTATTCCCTTCCACTTATGAGTCCTCGAACCACCGCCACACAGTCCATTGCTTAGTAGTGGTG
TGCTTGCCTACCTGTTTCATGGGAATGAGTATTTCTTGAAGGCAAGCGTCATGTAATTTTCTTCTATGATAAGGATATTTAAGGTGTTTATTGG
ACCCACTAACCATTGGTTGCCAAGTGAAGAGATGGGCTTCAGACTGTGATGAGTGATAAAGAGTTCTGCCTACTGTAGGAACCTGCTGGAACTC
ACCAGGTTCCACCTTTCCAAAATTACAGCCAGCTCTTTCAGTTTTAAAATAGAAAGAACTATATTTAACTAATGGAGTGCCTTAACATGTGTGC
GAGGAGGTATGAGTGTCCTACCATAGAGATGGCCTCAGTGGTTACCTTACCTGTGTTCACTCAAGAGCTAAAATGTTCTCCCCAACGTCATCTC
TCATCTTTGGGTATTTAAATATGTAAAATATGTTTTGGGGATGCCCTGGTAGAAATTTCTACACAGCTGTGCTTAAAAGGAGAAAAATCTAGTT
TGGGTTTCTGTTCAAGACTAACACCGCTGGGTTTTTCAACTGCACAGATGGAATTGCAGTAAAAGTCTCTCTGTTTCTTTTCTACAGCCATCTG
AGGGATCCGT6GTTCTTTCATGAAATCATTAATATTCTTAAATACAGATGGTTCCCGTTTCCAGAGACATTGGTCCCGGGGTCTGTGAAATTGT
GTTTATTTTCATTTTGATGCCACCATTTCTCAGGCAAAGGTTCCAACTGCTCTCCAAAACTTTAAAGAGTGCTCTGTCAGGATACGTAGTGAGG
CAGCTAAGCACCTCCCTGGAGTGCTAAGTGGTTCAGGCAGCGCCAGATGCCATGGAGCCTGATGAGTGTTCTAGGGGAACCATGTGGAAGGGAG
CTCTCCCAGAGAGAAAGAGAAGGGAAAGAAAGAGGGACAAGGGGTGAGGGAGGCTGCCTAGGCTCTCAGGGAGCCTCTACCTTCCTGCAGTCCT
TGCTAACAACGTCTTCTCCTCTGCAGACCCCACACTGTGGACACAGGAGCATGTGAGGCAATGGCTGGAGTGGGCCATAAAGGAGTACAGCTTG
ATGGAGATCGACACATCCTTTTTCCAGACAAATGGATGGCAAGAACTGTGTAAAATGAACAAGGAGGACTTCCTCCGCGCCACCACCCTCTACA
ACACGGAAGTGCTGTTGTCACACCTCAGTTACCTCAGGGAAAGTAAGTGCCGCCCAAGTACCCAGGGCTGGGAGGAAGCTTGGCATGGAGCCAA
CCCAGGGAGAGGAAGTCAGTGCTGCCCCTTCGTGTTGGGCAGATGCCGCCGGAGCAGCATCCTGGGGCCTGTAGTGTTGCCAAGGTCACGTGGG
CTCCTACAGACCCTGTGAGGGGCAGACCCAATCCTGAAGTGTCAAAGGAAGTAAGACGAGAAATGGAGAAAAGTAGAGAGCTGTGACCTGGAGG
GGTCTGGCAGAGGCTGGTTACACTCTTCCCATGAGACCGCCTTTCCAGAAACTATTCCAATTTGAAAATAGATAGAGATGGGTGGTGCCCTGGC
TGGTGTTTTCCTAAGTGATTATTTAAAATTCCTCCAGGAAACTCTAGATTCAAGGCTTTTGGATTTAAGGTGTTGAAAATAATTTACATATTAG
CTTCCTCTTAATCTTGGGATACTCTGGCACACCCTAACCCCATTTGACAGGGCTGCTATCTCCACACTTCCTTGATAGACGTAACCAGCCAAAG
GTGCAGGTGTGTTACGATCCATTGGAGGCTTTCTTCTGGTACAGCCAAGAAAGGAAAAGAGCTTTGGAGGGCGAGCTTAAGAAGAAGATGAGCA
GGCAGTTGTCCAAGAGATTTATCTAGTATGAGGTACTCTAGCACCAAGCGCCCATCCGTCTGATATGAATTTCATTTGATATGAAATATACACA
GAGCAGAACAATGGAATTATTTTAGTCCTGCTGGTAACCTCAGCCTGACTGCCCTGCTGGGAATCCACGCCACGTGGTATATCAATATCACTCC
TACCCCTAAGTATGGAACGATTGTCCTGGGGCCTGAAAAGATGGTGCTAGGATCACAGGAACACAGGTTTGGGCTCACACTACQTAAGAGCATT
CTAGCAGCCCTGTGCAAAGACAGGGCAGCCTTGAGATGCTATTTGTTGTGAATGGCCAAGCCAGCTGCGAGTCTTGCAGGAGTTTAGCTTTCGG
CAGGGCCTTGATGATTTTAAGCCCCCTTCGCAAGTTCACATCCTTAGACAGCAACTCTGGGACCTGCTTTTCCTGGACTGGCTGGCTCTGGGTT
GGTGAGCTCTGTTTGCTCGCCATCGTTCTGTGGACCTTTAACTCCCCATCTTTGGAATCCACTCTCCCTTCAGCGACAGTGTTCCTCATGGGGC
AGGCGCAACACGTGCTGGCTGGAGAAATGAAAAATCAAAAAACTGGCTTTGGCAGTTTCCACTGGTGCCTCTGTGCTCTCTGGTCACCTTTCCG
AGGCGTCTGGTAACTAGTGCGAGAAAGTAGATGATGCAGTATTTGTTAGCTCCGGAGCAATTACCTCATGTCTGAGTCTTGCTGAAGTCTCAGG
TCAGCCCAAGAAACAATTTCAAAAAAGGGGACAATTTTCCACCCAGTTTTTCAGAGCTTTGGATAATTAGCACTTTCTAATTCATATTTTCAGC
TTTTAACFGTTTGTGGAATTTTAAAMATAAGGATCTTTGAACAGAGTCCTGCAGTGATCTACGCAGGCCAGGGTGGGTGTGTGGACGTTTAGCA
TGACAAGGAGAGACCAGCAAAGCAACCTGATTCCAGCAGGACTTTGCAGGAACCCTAAGCCCTTTCCTTCATTTGCCAGGAGTAGAGAGGACAA
CAGTAGCATAGACAGCATGCTCTGAACTGTTAGTTAAAGGGCCCAGTGCTTCGAGACTCTTCGGCGGATGCCCTTGTTGCGAGAATGTTCAAGG
CATTCTCTCCATGTGGGGAAAGCCCATTCATGCCATATGACCCCGGGGCCCTAACCAGTCCATGAACAGCCCAAGGTCCCCTAGTACTTCCTGC
CACCGAACAATGGGAACACAGATGCTGAAGTGAGACAAATGCCTGACAAGGTGGTCACTGGTCGCCAGTAGACAGACCTTCTATTGGAGAAGAG
CACATAACAAGGTTCAAGTGGAGGTGCAGAGGATCGCATTTTGGGGGGTATAGCATGGCAAGAAGCAAATTGCTTCTTTCTTTTTAGTAAGAAT
ATTTATAGACAGAAAGTGCCCCCAAATCAATAAAAATATTAGGTTTCCCAAAAGGCACTTATTAGTGGAGCATATTTCCAGAGTGGAGAGGAGG
TGGAATCCAGCTTGTATACTGAAGACATTTGAAAATGCAGCGCAGATATCCAGAGTTGAGGATGCTGGCCAACAGTAGTACATCCACAAGATGG
GAAGTAGTTGTTCTGCCATTGAAATGATTATTGGGAAACACCATGAGAAGTGCTTAGGATACAATGTTGGGTGGAAGCTCATTATAAAGTAAAA
TCTGTGCACTGATAGAAAAAACCATGTCTGCCTGCGAAAGCAAAAAATTAAAATAATCGTGTTAAATTAAAAAAAAAAAACCTTGCCCCAACAT
GGTATTGAAATATCCTTTTGAGGAACCTAGACTTAAAATGATCCACTGTCACGGAAAAGGCTTTTAGATTCATGCTGCTTTTTAATTGGTGTCT
ACCTTGGGGTTCCAGCCTTGGGGACCCTCTAGTCTGAQGTGGTGGCTGGCTGCTAAGCGCCTGCAGGAGGCCATCCGCTGGCGATCGTGTCTGC
TCTGTTTCTGCTCTGGATTGCTGGGAACTCCCCCGTCACAACCAGCACTPTAAACGCTTTTAAAGCCTTCAAACACTTTATTGTTTTTCCAGAC
TGACTTCCAGTGGATGTTTTGTGCTTAGGTGGAAGGCCAGTGCTTGCGGAGAGTTTGAGGCGATGAGTCAAGGGAAGGGGAGAGGGTGCTGATT
CTTTGTGCTTTATCATCTTGTCCTTTCATTGGAGAGGAAGGTGGGCCTCAGATATTTCCACTTTAGCAAGAGATCGATGGAAAATAGCTGAGAT
CCTCACTCCCGTGGAGGAAGTACTGCAGCCTGAAGGAAAGTTGATAGAGCAGAATCGGGGACCTGAGACTGTGGCTGGTTGGAAAGTCAGTAGA
GTCCAAGTTGGTGGCACTTCTCTGATCTAATCTTTCTGGGCTTTATAGAAAGTCTAGAAAAAGGAAAACTTCATAGGATAAGAAGCTTTGGCAT
CAAATTTACATTTCACAAAAGGTGCTTGACTATTTATTCTGGTGGTTTGGGAGGGGAGCTAGACTTTTACATTTTAAATGCAAGTGTATTTTCC
AGGGTAGAGAAGAAATAGCAGTTCCCTGTCCCCCTACAGACCCTGTCTGATGGCCAGGCATGCTAGGGAGGTGCTTGTTCTTCACACCTGGGCG
TGTCAGCTGTGCCAGAGGAGCTGTGTCACACGCCACACAGGCGCCCACCCCTCCCCCCCACCACCACAGGAAATGCAAGGAGGACTGTGATGGC
TGGGGTGGCCACGGTTCTTGGGCCTAATTCCCATGAGTCCTCGGAAACTCCAGGGTTTACACCTGTCAATCCCAGCACTTTGGGAGGCCAAGGC
GGGTGGATCACCTGAGGCCAGAAGTTCGAGACCAGCCTGGCCAACGTGGTCAAACCCCGTCTCTACTAAACATACAAAAATGAGCCAGGTGTGG
TGGTGGGCGCCTGTAATCCTAGCTACTTGGGAGGCTGAGGCAGGAGAGTCGCTTGAATGCGGCAGGGGGAGGCTGCAGTGAGCTGAGATCGCAC
CACCGCACTCTGGCCTGGGCGATAGACCGAGGCTCTGTCTTCAGAGGAAAAAAGGTTGCCCCCTTGTGGACAACTATCTACAGTACCCATTTTG
AAGCTGAACCCACTGAGGCCCAGACAGCTTCAGAAATTGGTTCAAAGGCATCCAGCCGATTGAGAGCACCTGGCATCTCAGACTAGGGCCTCTG
GAGTCCCAGTCCCTCGCCTCGCGTTGCCGACCTTCCCCCGGCCCTGGAGACAGCAACCCAGCCTGCGCCACACAGGCAAGGCAGCCTCACTCCC
GGCCTTCCACGTGGGCAGCCCGGCCACTTCCCCCACAGCATTTTCTGTTGTCATTTGTACCAAGAGATAAATGCTTTCCAAAAACTTTCCTGTG
GCCTCCTGGAAGGAGGCCGATTAAGCCAAACCTATCACCACGCTAACTTTGTCCTTCTCTGGGCCTCACTGTCGGCCCCACCAGTATCTCTCCC
GGACACACCCCGTGTGGCAGAGGAGCCTTCGCAGCAGCWGCTGCGGGAACGAGATTTGTCCCTTCTCAGGCCAAGGCGGGGAAGGACCTCGAAG
AAACGGAGGGCGCCGGGCTGGCAGTTCAGGCTCAGGGCCGCTGGGGGGAGTTTGCGCAAGGCTGGGAGCCCCGAGGCCCCGGGGGAGGTGGGGA
GGCAGAAAGAAAAAGAGCCTTTTTTAAGTTTTACAAACAAGTTCGTTTTGTATGTCAATCTGGGTCTGACCTGGAAAAAAAAACACTAAGAAAC
TGCGTCTTCAAACAGTGGCTGCTCAGAAGAATGAAACTGCGCGACCGGCAGACCACGGAAAGGGTGTCTATCTTCATTCCCATTTTTAATTTAA
ACTTTAATGGAAAACACATTTTGCATTTTCTGGTCTTTCCACCTGCAGCCTCAGATGGTCGGGCAGAGGTTTTGGGTCTGGGCAATTAAGCTGG
TGCAATCTTATCACTTCCAGCCCTGGGTTTGACGGGTGAAGGAGAACAGAATGCCAGCTGAACAAAGCGCCAGGTTTCCCTGGACCCAGTAGTC
AGCCAGGATTGTTGTTTAAACATCCCAGAGCCTTGCACCCTCCCGCTCCTGCACACTCAGCCAGAGCTGTTGATATAGCAGTGCAGCCCAGAGA
AAAGAGAGACCTGGATTCAATAGGCTGGCTGTGACGCGGCACTGGTGAATTCTATGGTCACAGCGAGGCAGGAGACAAAGGCTTTGGCACTGGG
ACGCTTGCGCCCCCTGGCCCAGGTGTCCCCGATCAAAAGCAGGTTAGGTTGAGGCAATGTGAAGCAGCAGAAACCCTGATTGTTGGGGTTCTAC
CCCACACCTGCAAAGCCTCACACTGCTCACTCCCCTCTCCTTTCAGATCCAGGTACTGTCTCTAGCAGCACATGGATTCAAACATGGAATGGAA
AATGATCTTTTCCCCATGCTTGCACTTTGGCAAGGCCATTCTATTCTCAGCAGGAGCTACTTTATGCTTTTTAGAGCAAAAATGCCCTCAATAA
ATAGATATATGTATATGCATACACACCCATATACAAATATATACATACAAACACATATACAGAAACTTACAAATAACCAAGTAGGCAAATCTTA
TACTTCAGTTTGAACTGAAGTACAACACAGAGAGAAGTGCAAAAATCATGAAGTGCGCTACGTGAGACATTATCGTGACGTGTGACCACCCACT
CCCATAAGCACCTCCCGGGCCAAGGAGTAGAATTTCAGCCCCCTGTACCCTCTCTCCACCCCACACCGCTTCCTCCACAAAACTAAACCACCAC
CCTGCTTCCCAGCCCCATAGGTGAGTTTTGCCTATTTCTACGATTTAGCCTGGAGGCAGTGGGATCTGCCAGATTGAGGACAGGTTTTGTGAAT
AGACCACGATGGGGCTCCTGCCTTCTGCACTCTCTGGCTGTCTGATCCTGGGAAAATGATATGACTTCGCTGAGTGTTGCTCCTTTCTTCTGTA
AAAAGGAATAAGGGAATGCTGGCTCTGTATCGCTTATGCAAGGATTAAGTGAGAAAATAGATGGAAAGCGTCTGGCACACCGTGCATGTTCACA
GAGCACTGCGTCCCCACCACCACCTTCATCATGGCCTGTTTACCGTGATGTGCTGGCATCGGGAAGGTGGGGGAGCTCAGCAGCACAGGCCTGT
GGCAGGGTCAGGCCAGAAAGACCCTCAAGGCAGGAGAAAGCAGTCCAAAGCCACAGAGACGGAGGCCAGACTCCAGGAGGACCTGCCTGCATCA
GAACAAGTTCCTTAAGAAAACAGAGCCTGCGGAACTAATGAAGTAGAAATCGGAGAAGAGGAGGQAATCTAGGTCCCTTGGGAGAGAGGCCAGA
TGCCCCCAACCAGCCCCTCCTTGGGGAAGGCAGCCTCTCCTTCTAGGAGTGAAACTTGAACCAAGAACAGTCTTTAGGGCAAAATCCTTTTCCT
AGGTTGCCATGAAATATGCATTTCATGCAAGCTTTGGAGAGGTCAAATGACCCTTTCTCTCAGCTTTCTGGGAGTTTCCTGTCGAATTCATGTC
TGCCCAAAGAAGAACTTTCACCAATGGGGAAATTTAATAATGTGGTTTATCTAAGGTTCGTGCTTTCGCCTACATCTGCACTAAATGACTCAGG
CAGTTTGCTCCTCGGAGACCATCATTTTTCTTTATTTGCTCCAGCAGTGAGGTTTCAGCCTGAGGGAGAGAACGTCAGGCACGGAGAGGCAAGC
ATTCTTGGTCCTACTTCTGGGATTTTTTTGTATTCCCTCCGAACATTTGGTTCCATTTCTTCACTTCCTTCCAGTCGTGCCGGATTATTTCTTA
TTGCAGCAGAACCCATTCATAACTTTAATCAGAGGACAGGACGAATGTTCAGTAACGCATGTATAGAAGAGACTATTGTATGTATATTGTCACA
TCTGTGGGAGAAAGGATGTAAAAATAGTAACGATCATAGCTAGCATTTATGGAACACTTATGAGCCAAGGTCCATTCTGAGCACATTGCATGCA
TTATTTGATTTAATCTTCATAATGCCATTGAGTGGTGTTAGCCCATTTTACAGATGAGAAAACTGCTGGCTCAAAGAGATGAGGGAATCAGTGG
AGGGCACAAAGCTAGTCAGTGATAAGAGGCAGGTCTAATTTTAAAGTCTCCTTTGCTTAATAAACATACAGTCGTGCTTTGCTTCATGATGGGG
ATAGTTCATCTTCATGTGAATACCACAAAATGTACTTACACAAACCCACATGGCATAGCCTACTACACACTGAGGCTGTATGCTACAGCCTGTG
GCTCCTAGCTTACTAAGCTGTATAGCATGTTATTGTACTGAATATAGTAGACAACCGTAGCACAATGGCAAGTATTTGTGCATCTAACTTTTAA
AAGATACAGTAAAAATACACTATTACCGTTGTCGTATATGTGGTCCATCTTTGCCCAAAACATCATTTTTTGGTGTGTGACTATTAACAGGAAC
TTGTCCCAGCCTTACTTTGTCATTCGTATTCTTGAACATCGAATAACCAAATGTAGCTGGCTCTTGGATGTTATATTCATGTGACTGTCTCATT
TCTAAAATGGCATCCACATAGCCACACTTTGGCATACAGCGGGCTCAATACCACCCCCGTCATCCCCCTCCTTGTTTTCATCTGGAGCTGCTGC
AGAGCTAGAACTAACAGTCATATTCTAGGCAATTGGACAATTCTTATAAAATTCTCTTCCTCTGCCGGGTGCGGTGGTAATCCCAGCACTTTGG
GAGCCCGAGGCAGGCACATCACCTGAGACCAGCCTGACCAACATGGAGACCCCGTCTCTACTAAAAAGATACAAAATTAGCCCAGCATGGTGGC
GCATGCCTGTAATCCCAGCTACTGGCGAAGCTGAGGCAGGAGGATCACTTGAATCTGCGAGGCGGAGGTTGCAGTGAACTGAGATCGCGCCATT
GCACTCCAGCCTGGGCAACAAGAGCGAAACTCCGTTTCAAAAAAATAAATAAATAAATTCTCTTCCTCATCGTCTGGCTTTGGATAGTGCACAC
ATTTTCTCTGAGGCACACAACCAAACGCCCACAGCTGCGTGAGGCGGCAACTCCATTTCGGCATCCCTTCCATGACTGGAGACTAAAAATAGGG
GTGTGTCTCACAGCCCATTCCCACCCCTGGCACCCAAGCCAGTCCACTCGGCCACAGGGAGCGGGGAAAGGGTTGTTTAGAGAGAATTTTCTTA
GCCTCGAATACATGTGTTGAGCTTATGGTATGTTTTATAAAAGCAGATCCCAGCCCTCGTTTCAAGGGCTCTTTACATTTCCTTAGGAGTAGTC
TCAAAGAGAATGGCAAAAATGCCTAAGTTTCACAAAATACAACCTTGAATTCACTTTAAACAAAGGAAAAAGTCTAGTTTTAGGAAAGAAGGGA
GAATCTGTTTTACATGTAACAACAAATTGCTTTTTCAAAGTGCTGGGTACAGACACGTATGAATTGAAGTTTAATTTCGATAAGATTGGATATC
AGCATTCTGAGATTACAGAAAATTCAACGCGTGACTCCTCAGCTTCAGTGCGTGCTCTTACACATAGGTGAAGGGAGCAGTAAGTTCCTATTGG
AATACTACCACTCACCTCCTTCCCAACTCCCATTGCCCCCTCCACCAGTAACATACCTGTGTACCTTACCCTCACCTGTGAAAGGACCCATATG
TACATAAACCCAGAAGATACCACAGCAGGAAGGAGTGATACTGGCTTCTCCTCCAGGCCATGGCAAAGTACAAGCTGCAGGCCAAATCTGGCCA
AAAGTGGCCCATGGACAAAGCAAAACAAAAACTAAGAAGACTGTGCAACAGAGATTGTAGTTAGCCTATGAAGCCGAAATATTTACTATCTCCT
TTTGATAGAAGAAATCTGCCAACCTCTGCTCTAGTTCAAGAAAAAAAATCAAAATCTAGACATGTCAAGTCACTCACCAACTGTCACATGGTGA
GTTAATAACAACTGGAATAGACCCGGGTTTACTGATTCTCAATGCTGTGTTCCTATCATGATTATGGCACAGACCACACCGCTTCAGAACTATG
AATATGGCAGCTTAGGGGGCGTGGTGTTTGTTTCCCCAGAGAGCATTTCATTCCAAAGTATTCTGTCTCTTCACTCCATAAAGCTATTACAGGC
CTGTATGATTGTCTCCAACCAAATGGATTCCATTCGCTTTTCCTTTCTTCCTAGGAGTCCTCTGTCCCTCTTCCCCTCTCCCCATCTCTTTCCC
TTTCTACTCCCTCCTCATGTCATCTCCTACTCTTGATCTCAGAAGAACATTTTGGTTATAACCTGTTTATGTTTTGCCTCTCAGGTTCACTGCT
GGCCTATAATACAACCTCCCACACCGACCAATCCTCACGATTGAGTGTCAAAGAAGGTAAGTTTGTTCTTTTGTGCACTTAAAATTTTCTTCTG
TACCAGACATCACACAGGCCCATGCTGTTAAGGTTGTTGCAGAAAACCAGCTTGCTTGTTCCACTCAACTTTTCCTAGCATACAAAGACAAGCC
AAAGATTTTCGCCACTTGTGCTACCAGTTGCAAAAATACAGTAAAGAAAGTTTTACAGTATCTTGTAATAATATCACAAAGATACAACTTGAAG
TTGTCACTCCATGCAGGAAAACATGTTACTTTTAGAAAAAAATGAATGCTGCTTACTTTCAGTTGAAAGTTGGGAGTTTTCTCTGATGAAAAAA
TAATGAAATGAAGAGTTCAACCATTCCAGCATGTCTACCTTTAAAGCTTTTTTTCCTAAGAAATTTTAGCAGCCQCTCATGCTGGCTCACACCT
GTAATCCCAGCGCTTTGGGGGGCCGAAGTGGCTGGATCACTTGGGGTCACGAGTTCGAGCCCAGCCTCGCCAGCATGGCGAAAACCACCATCTC
AGCTAAAAAATACAAAAATTAGCCAGGTGWGGTGCCAGGTACCTGTAATTCCAGCTACTCAGGAGGCTCAGACATGAGAATCGCTTGGACCCAC
GAGGCAGAGGCTGCAATGAGCCGAGTTCGCGCCACTGCACTCCAGCCTGGQCGACAGAGCAAGACTCTGTCTCAAAAAAAATTTTAGCGATGTC
AATTCATTTTTAAAGTGGTGGCTTAAGCCTTTGGTCTGGTTTGGTTTGCTTTTGCTGAGAAAGAGAAACTTCCTCGGCAATCATTTTTGCCAGT
GAAGATTTTAGTACACGTCACAATAGAATTCCTCAGCCTCTTTATGAAACATCGTGAGGAGTGGTTCAGATACTGTTAGATTTCAGGCATTGAA
ACAAGTGAATAATTCATAGTACCTGGTTTAAAGGAGGAAAAGATGTGAACAGAATAATTATTAGGAACCAGAGAGAGACTGGAAAAATATTTGC
GCGGCATAGCAAAAATTCCACAGAGGAGGGGACTTTTGAATACGAGCTTGAAGAATAATAAGAGTGTGGCAGGCACTCTGTCCTCCTTGGCTCG
CTGTCTGTCCCCAGTTTTTCTTTAACTCACCCTCCCCAAAATATATAAGAAAGGCAGAAGTTCTCAGGATCAGTTACATATAGCACACCACTGC
AAAACCTTTCAGAGAAACAAAGGCTCTTCTAAGGGAAGGACCATTTGGCTTAAAATCCGCATGGGGTGTGAAGGAAACAGAGAGCCAAAGGCAC
TAGCTGGCTGTCTTGCCCAAGGTTACAACTTTGATCACACAGCACTTTGTGTTTCCTGTTAACAATTTATAATTTAATAATGACCAGAACATTT
AGTGGAAGAATTATACAACATAATGAATCCGCTTTTAAACATTTGTGTTTTTCATTTGTTTCCTTCTCTCCTATCCCACCCTGAGCCAGTCCTG
TTTCTGCCACTGTTTTTCCTTTCUAAATCAGATTTCTTGAGTTTTGTCATGAACGCTGAATTTCTATGGATTCATTCTACATTGTGAGTCAGAT
TTCACTTTTTGACCTATTTTAATATGCTGTTGCAAATGTCTGTAAGAGAGAAAATACATTTTAGTGGCAAACGCATTAGATTGAAGGTCAGGGC
ACCTTTGGCTTCCTGTGAGAAAACCACTTCAGGCCCTGAACCTCAGTTTCCTGCAGAACGAAAGAGAAGAACTCTGTCAGCAAGAGGCCAGCCC
TGACTCTGGAGATCTTCGCCTCTAAGGAGATGACAGGAGCACTCACATTGCGAGGGCTCAAGAGCCACTGTCCTTTCATATGTTCTACATACTG
AGTTTTATTTCATTTCTGGATAGGGATCCACTTTTTGGAAAGGAAGGAAAAAGAACGGGAMAATGACTAGAGCGACAGAGAGAQAGGGAAAGGA
AGGGAAGGGAAGGAAGATAGGGAGGGAGAAAAGCAAGGCTATAAGGAAGGAAGGGAGGGAAGGAGGAAGGGAGGGATGGGAGGACCCTAGACCT
CTGAACTGCTGAGTTCGGATTTTTGTAGTTTCTCCAACACTTCAGGCTACAACCTGCTACCAGACATCAGAATTAGCCACTGAGGCTCTGAAGC
AGAGCTAATGAAATATTTTCTGATATTGGGCTTCAGATATAGGGTTTAGTTTAGAAACTGGTTTAGCCAAGTTGAAATGTGTTGCTAACCATCT
CCTCCTCCTCCTCCTCCTCCTCCTGCTGTCTTATTGTTCAGCCGTGTCCTCTACCTGCCATACACATTTCCTTCCCTTGGCCCTTTTGTGTTTA
GGGAGGACGTTTGTAACACTGCTGGCTCATTCAGGCTTATTGCAGGAGCAGCTCCCTCTTGCTGGCTACCAACCCCTCTTCTGAAATCAATTAA
CTGTAGTATCCAGGCAAACACACGCTGCCAGGACCTCATTCAGGTGGTGCCGGCATTTATCTCCCAACATCGGGCCTGGCCTCTAGAGACGATT
AAGCCTGTGCTTTGCAGGTGCATCTTTTGTCCCCAGTAAAGCTCTCCACTTGAGCTGTCTTGCTCCTCTATGCCTTCCCCTTGTGAGGTGGTCT
GAATCTGTCTGAACGACCCATCAAGCCACAAAACAAAGTACATTAGCAAGACAAAGCTAAGCAAGCCCTCCCTCTATCCTCCCTGCCTCCTCCC
CACCTCTCACTGGCTCTCCTGCTCTGCTCTAATCACTATCTATTAGCTATTAGAAGACATCAGGCAACCTTGGCTCCATCTTCTTTGAATAAGA
CACACACACCCCCAAACAAACCCAGAGTTCATCAAGAGCACCACACCTCTGTGTGTGTGTGTGTGTGTCTGTGTGTGAGTGCACACTGGTGCAG
TCCACAAAGGGCAGGTGCTGGCATCAAGCACAAAGCAAGGAAGTTGAGGAGATGCTGTCTATTAATGTCTTCAAAGCACTTATCAAATTAGAAT
GGCTTTTTGCTTTAAAATTGTATAATCACCCTTCCAGCTTGTCTCTTGAAAATGGCTGCAATCTCTAGGGGTTATTTGTGCCCAGGAAGTAAGA
GTAGATTTTAAAAATTAATTTAGAACAATCTTTAAGCCCCTTTAAATTAAATATTCTATGCACATGGATTGGATCCAGCTAGAGCAGTTATGCT
CTACCTTTTAGTTTATCAAGGCTAAGGCACTTAAAAAATAAACTAGGATAAAAGCTATTGTTATCCATTTACAATAGCAGCCACAACTGTCATC
TGTAAAGGGTTTTGGAATATATCAGACACCTCCACATGCTTTGCTTTGGAGCCTAGCACTGGGTCTTTGAAGTAGATATTATTATCTCTATTTT
GCACACAAAGAAAGTGCGGTTCAGCACTATTAAGTGACTCACTGAAGCGTACACAGCTACTAGATGGCAGAGCAAGGACTCAAACCAGGTTAGA
GGATTAGACTAGAACCCCCTCACTTTCTGACAAATACAGCCTTTCTCTTCCAGCTGCCATTCTGCAAAAGCAATACCTAGCCTAAATGTAACTT
GGGCAAACATGTGAATGTTTTGCATTACTTGATTTCTGTCCATGCAGTTTTCCCACTGTCAGCTCCATCTCCATCCCTAAACTTTTCTCTCTTT
CCAAAGATGAACCAATAAACAACTTATGCTTGGCACCAGCCCTTAGTTCTGACAGCACAGAGAATCACCAGGAGATCTTTCATAATACTGACTC
TAGGACAAACATTGCATGATTGCACTCATAGGAGGTAACTGTCAACTTTGTAGAGCCAGAAGGTACATGGTAGTTACCAGGAGCAGCTAGGAGT
GGAAATAGGGTGTTAGTGTTTAATGGGTAAGAGTTTCCATTTGGGAAGATGATAAAGGTTCTGGAGCTGGATGGTGGTATCGTTGCATAGCATT
GTGAATGTACTTAATGCCACTAAATTGTACACTTAAAATGGTTAAAATGGTAAGTTTTATGTTACATATTTTACTAAAATTTTGTAAATGCAAA
AATTAAAATAAAAAAATACTGATGCCCAAGTGCCAAAAAATGATGCATTAATTCAGAACCTCCAAGGGTGACTCCTGGGCATTGGAAATGTTTT
AGAGCTCTCCGGGTTATTCATATAAGCCAAGCTGCAGCCAAGCTAGAGAACCCTGCTTTATAGTAATCAGAGACAACTAGAGAGAGATGAGCCC
ATGTTAGAGCTACTCCAGATTTTCTTGGAGACACACTGGGCGGTTGTCTGGACCCAGATTGTCCTAGAAAAGGAATAGCAGGCCCTACTGGTAT
ATTGATAAAGGTATAGGTAAGTGAAGAGACTGTACGATCTTGATTAGTTTTTTGTCATTGTCATTGATGACTCATTTTATTGATGAATGATGTT
ACTGATTTAGACAACTGTACGTTCGTACATCTCAGAAATTTCTTTCTGACGTGTCCTCATTCAGTCCTCTAAAAAGTAGATATGGTGTTGGTAA
CGTGATTTTATAGGAAGGATACTGGGGTTGCAGAAGTTAAGTGGCCTTTTCAAGTGTGTAAGAGTCCTGGAGCCAGGGCTACTGACTTTCCACA
CCTTGTTCTCTCCCGTCCTCCAGGCTGCAGATGTGCGTGTGTCTGCCCTGGTCCCAGGTCAGAGCTTCCCTTTGTTGTTCTGGGAAAACCCTGC
CGGAGGCCTTCCCAGCACACTCAGCTGGCCCAGCAGTGTGCAACAATCAGCACATCTCTTCCTCAGAGCTTCTTTGCATGCCTAAGCCCTCATT
TCCTTAGTTCCTCATGCTGAATCTGTGTTAATACCTCTGACTTTCCAGACAGAGGATATTGAGTTCCTTGTTTATTATTAACTCCTGGAACTCA
TCAACCGGCCAAGGGCCTGTGGAGCACTGAGCTTCTAGCCCCAGCCTGGAGCCTTTCAGGACTGCCATTGAACACGGAAACTGAAAACCACAGC
CCCACTGCAAGCGGAATGGCCATCTCCAGCCATTGCCTTGGCTGATGGATAAAAAGTCTGTCGGCTTGCATGCTAAGTATGGAAAAGAGAGGGG
AGAAACTACCTTTCTGTGTCCCTTAGAGTGAAATGCCTGGCTGGTGCTGAAGCCCATGTTAACGAGAGGCTGGCTTCTCTCCAGGAGAGTCCTA
GGTGGCAGGAGCCATGCACATGGTAACAACAGCATTTTGGCAGCCTGCTAGGGTTTGCCAAGCACGTTCCTATCCATTATCCCGTTTGTGCCCT
CTCGTGCCATCACCATCAGATGAGAAGGCTGGTAGTATTATTATCCCCCTTCTATAGATAATGAAAACAAGGCACAGAGAGATTTCAGGGTTCC
TTAGAAGGCACCCAGGTCATGAAGCAGAACAAGACTCAAGTCCGGGCCTTTGGTGTGTTCTGTCCTTCCTCTAGGCTGCTTCTGGAATCATTAT
CTCACCATACTTGATTCAAGCATCTGACAACATCTTAAGGATTCTTTAATACTCTCAATTTACCCAAGGTGGGCACTGGTCTCCTATTTCAGCA
TTAGAAGGCACCCAGGTCATGAAGCAGAACAAGACTCAAGTCCGGGCCTTTGGTGTGTTCTGTCCTTCCTCTAGGCTGCTTCTGGAATCATTAT
CTCAGCATACTTGATTCAAGCATCTGACAACATCTTAAGGATTCTTTAATACTCTCAATTTACCGAAGGTGGGCACTGGTCTCCTATTTCAGCA
TTATAGCATACTGATTAAAAACTTGGGCTCTGGACTCAGACTGTGACCTGGACAAGGTGCTTACATTTTTCTGCCTCAGTTTTCTCATGTATAA
ATGAGGATAATTGTAGTCCCCCTTCAGGGATAATGTAAAACTCATAAATCAGCACAGTTTCTGACACACCAATAAGTGCTCAATAAAAGTTATA
TTTTTTTGAGATGGAGTCTCGCTCTGTCGCTCAGGCTGGAGTGCGGTGGCGTCATCTCGGCTCACTGCGTCCTCCACCTCCCAAGGTTCACGCA
TTCTCCTGCCTCAGCCTCCTGAGTAGCTGGGACTACAGGTCCCCGCCACCACGCCCAGCTAATTTTTTTTTGTATTTTTTAGTAGAGACGGGGT
TTCACCATGTTAGCCAGGATGGTCTCGACCTCCTCACCTCAAGAGCTGCCCGCCTTGGCCTCCTAATTACAGTATCCCGCTAATTACACTATTT
TTGAGGGTTTACCAGATGTTAACACCGTACCAAGGGTCGTGAAGGATACTATACATAGATAGCCAAGCTGCTTTAAAACTTATTCGGGGGCCGG
GCATGGTGGTTCATGCCTGTAATCCCTGCACTTTGGGAGGCCAAGGTGGGCAGACCACCTGAGGTCAGAAGTTCGAGACCAGCTTGGCCAACAT
GGTGAACCCCGTCTCTACTAAAAATAAAAAATTAGTCAGGTCTGGTGGCATGTGCCTGTAGTCCCAGCTACCCCGGAGGCTGAGCAGGAGAGAA
TTGCTTGAACCCGGGAGCCGCAGGTTGCAGTGAGCGAAGATCACACCACTGCACTCCAGCCTGGGTGACAGAGTCAGACTCTGTCTCAAAAAAC
AAAGCAAAAAAACAACTATTTAGGGAGACACACCTAGGAAATATGAAGGAAATATAAAACTGCATGTAATCGAAAAACAACAATGCATGCTACA
CACAAGAAATTTTCTGAGTGTAATGAAGAGATGGAATCATTAATGCTCAAGCTGAACATCTCCTCCAAATCCCTTATAGAGAACAGGAGAGATC
TGAGGCCAGAGAAGCTGTGGACCCAGCCATGGTCACACAGGGAGTGAAGACAAATATATACAATTATTCCTATGTAGGCAGATCTTATAGATAC
ATGATACCTTAGTCAGGAATTGACCTAAATTCTGTGATTCATGCTCTCCTCCATTCATTCATTTCTTTCTTATGGAGAATCTTCTCTGCATAGA
ATACCAGGCTGAGCCCTAAAACCATGGTGAACACAGCACACTACCTACCCTCAAGCAGGTTCTAGTCAAACAGAGAGACAGACAGCTAAGTAAG
TAAGGAACTACTGTGCTTTAGAGTGAGGGCAATGCACGTATATTACCACGTCTTTCAATTTTCCCCTGCATTTAGCTCCTGTGTGGAPGTCCTT
TATGATTTTAGAGGTCATAGTAAAAGATGGAGGCCTCTAGCAAGCTGCAGCATGGCAGGGCTGGAAATAGATGTTGAAGCAAAGGGAAGGAGGA
AGGCACCAAGGAACCAGCGTGTTTGTTGGGGTGGGTCTGTCAGCTTTCAAGTTCGCTTTTGGAGACAGGAGCCCACGTGAGCCCTTGCAGGAAA
TAACTCAGGCCTGGGTGTTTGATGTGGGTTTCAGCAAGACACCCTGGGGTAGAACCTGTGCACCATGGCTTCCTGGTACCTGGAAAACCCGGTG
CAGTTGGATGTTTCATTGTGAGACAGGAAGCTCAGCGAAGGAGCGGAGAGCCAGGGACTTGTGTCAAAATCATTTGGCCGGGGAGGGCGGACTA
GGTGGGCATGGAGCTGAAATAAGACTGACCATGAATTCATGATTGTTAAAGCAGGGTGATGCGGCCTCAGGGTTCATTAGACTTTCCTCTCTAC
TTGTATATATCTTAGAAATTCTCCGTAACTCAAAAATGCCTCAAGATATCTTTGCCTTATTTCTCCGGGCGAAGAGTTTCCTGAGGAGGGGAAT
CTTTGTCCCTCGCGATATGCACTGCCTGTGTCAGGCTGATGCGCTCCCGGGTGCAGTACTTGGGAAGGCGGGCATTTGGCAGCCTTGTGTGTGT
GTCTGTGCAGGAGGGCGTGAGCTGGTGCTCAGTGTGGAGGTGGGCTGTGTGTGTTGCCTTGTAGAGTGCAGCGTATGGGGGCTAGGTCTGGAAC
ATGTGTACCCAAAGGAAAAGGCCTGTAATACTGTGAGGTAATCTCCTGTTATTTTTAGGGAGTAAATAACATCTCTCATGCAGCAGGTTGTGTG
ACCCCACAGGCCCCATCTACGTCAGCTCACACACACTTCAAATTGGAGGGTATCGCGGCCTCACAGGAGAGCCTCTGAAGAACATTGTCATTAT
ATGTTCTTCCTTTATGATGAAATGTGCCTAAGCCATTCTTAGCAAAAGCAGGGATGATTGCTGGATGTGTGTGTGTGTGCATGCATTCGTGTGT
CTGTGTGTGTGTCTGTGTGTGTGTGTGCACGCGCGCTGTTTCTGTGGTCCAAAGGCAAGATTTATGATTATGGGATAGACAGTAGCTTGCTCTC
TCTATTTTATTTTTTAAAAACTCTTTTCCTTTGAAATTCCTGAGTTAATGTTCTGTCTGCTTTCTATCTGACCCTCTTCGGAGAGTTCCATTTT
TTTTTTTTTTTTCACGAGAGAGAGATTAAGAGAGAGAGAGACAGAGACAGAGAGAGAGAGAACCAGGGTGACAGAGAGAGACAGAGATCGGGGG
AGGAGGGAGAGCGAGAGAACTGGAAACAAAGACTCGTTGTCTCTAAGCTGGAGCAGGCAGGAAACCCCAATCCCTCGGGAACTTAGCCAGCCCG
CCCTGTCCTCGAGTCAGACTCCAGCTCTCTGAATTCAATTTGACAGCTTCTCTGGGACACTTTCTAAGTAACCATTATCCTTCCTTTCTCTAAG
TAAAAGGACTGTTATTTCTTTTCGTGGGTGTGCTGTGCGAACTGCTATTGCACACGTGTGGCTCACACAGTCACTAGGACACACTTTCCTCAAA
TTATTGGAAACAGCCCGTTTCCTAAGACAAGTGTAATTTGTTCCAGGATAATAAGGCAAAAAGACTGAGTATCAGTCCACTGCCTTATTACAGG
TGATCATTCTCCCCAGTTTCTGTGTTAATTGGTGGATTAATTTAATGAAACGCATCACCATACATCCCACTGTGGAGGGAACTCAGATGTCCTC
TGATCATTCTCCCCAGTTTCTGTGTTAATTGGTCGATTAATTTAATGAAACCCATCACCATACATCCCACTGTGGAGGGAACTCAGATGTCCTC
AAAGCTACCAGAGGTTGTGCAGGCCACGGGCACAACTAGGGGTGACCACCTAGAGTTCAGAAGGAGGCTGTCTTCTGCCTGGGAACAGGGCGTC
CTGGGGTTTCTTTTATTACTCAGACCCAGTTGAGTCACCACCATAACCTGAGGGACTGTGAGGTGATCACCTGCCCTTCAAATGTGTTCCTATT
GGAAGACCATCCCGATTCAGCTCTGTGGGACCGCTAGAGACAAGTTCCAGAACTGTCTGGATCCCCCTGGAGCGTAGAGGTGTGATAGTTTTGC
TCAGTAATACAGCTTTGCCCATTTAGGTTCTGACCCAAAGACCTGTCCTTCAAAACATGACTTGGTTCCTGTCTACTCTATCTTCACCACACTT
TGGAATAATGTTTCCCCATTCTTCCTTTGCCGGGTATACCTTTCATCCGTCTCAGTCCCCACCCCCAGTCCATATTCTAGGAAACCACACTCAC
CCTTTAGGGCCGTGGTCAGATCACACAGCATCACCTCATTACGGTCTTGTGCACCCTTCCCCAGGATGCTTTGCTTATACCCACTGTTTTACCA
ATACAGCATTCAATAGATTCTAGCCCAGCAGTCTTCTGTCTTGTGAGACGGGAAGCGTCTTGGAATAGGGACTATTCGATGGATGGATGTTTAA
TAATAGGCAAAGTACCTTAAACTGTTCCCCAGCAGTCTGGTGTAGAACTAACCTTTGAGATGTTTCTACAAGGTGGATGTTTTTGGCTCTATCC
ACTGCAGCTGCTGGTGTGGCATTGCCTTTCCTCCCTACCCTGCCCTAAATTTCCATTGCATGGGACCCCAGGGTGGTTCCTTGGAAGCTGTGAT
CAGTCCCATCCCTGAGCAGGGAAGCAAAACTGATAAAGAGCAGATTTCGAATCACAAGATGATACCACCAGAGCCCCTTCTTGGCCCGACACCA
CAGGATCCTGCTGCCAACTGGACTCCACAGGCCCTTCAGGTCTCTGCCCATCAGGAGGAGGCTGAGGACACGCCAAGCCGAGGAAAGGTGACGC
TGCAGGGAGAATGTACCTTCTGCTCAATTCCACCTTCCTTTCTTCACTTATGCCCCAGACAGCCACAATAAGAGCTAACATTTGTTAAGCACCT
ACTATGTGCCAAGAAATTTCCTATTCATTTCACATTTATCCTTCATAAAACCTTGTTAAGTTGGCACTATGATTATTCCCATTTTACCTGAATC
ACAGGTATGATGTTAAGCCTGAATCACAAAGCTAGTATGAAACACAAACAGAAGAGAGCCTGTTCCTGTAGTCCCCTCCCCACCTAGGCAATGG
CAAGGGCTCCAAAATGACCCTTTCCTACTTCATCTACCACCCCCATCATGCATTTCCCCCAAGTAGCTGATGTTGTCTAGTTGCTAAGATATAT
TCATCTATTGACTCCTGCTACCATCTAAAAAATCAGGAGCCTGACCCAGTGCAATCAATCTTATTTGCATTTCAAGAAGCAATCTAGCAATTTA
GAATTTTAGAAATTCCACTATAAAGAGGTTGTTTGCCAGGTGGGATGGGGCGGTGGTAATGGAGCTGCAAAGTATATAAAGAAAACATGCTTTG
TCCACGCTTATCAGTCAATTCTTTCTTTCTATCCATCCCTGGAGAGAGAGAGGGAGAGGTGAAGCTTCTTCCATGCCACATGGAGACAACGGGC
TTCAGACATCTTCTAGTGAAAGACCAACGTATAGGAAGCATCATTTGTGGTGGTTTCACAGATTTTTTTTATTGTTACGCTTTATTATTTATAT
AATTATACACATATCCTTTTGTGTTTAATACTACATTTAAGAGGATAAAGGAAAACAAGAGATTTAGAAAACAAAGTATTATATTGAGTAACAC
TCTATTGATGAGAATTTCAATAAACTGAATTTAAGAAACCATCCAGGCACAGTGGCTCACACCTGTAATCCCAGCACTTTGGAAACCCAAGGTG
AGAGGATCACTTGAGCCCAGGAGTTCAAGATCAGCCCTGGCAACACAACTAGACCCCATATGTACAAAAAATAAACAAAAGTAGCCAGGTGTAG
TGGTCTGTTCCTATAGTCCCAGCTACTCCAGAGGCTCAGGTGGGAGTATTGCTCGAGCCCGGGAGATCGACGCTGCCATGAGCCGGGATTGCAC
CACTGCACTCCAGCCTGGGCAACAGAACAAGACCTTGTTCTCAAAAACAAAGCAAAATAAGAGTGGGGGTGGATAAATTCAATAAATTTAATAA
ATTTATTATTTGAGGGAAATAAAGTTTCCCTCAAATAATTTCATGGGTCTATCAGTTTCATGGGTAATCCTCTGCCTCTGTAAGCTTCATAAAC
CCATGGTTCCCAAGGAGAAGAAGGAAGAGATGTCAAAATCAACTTAAAGCATCTTAAAGACACGTCCAGGATGCTCCAGCTGCCCCACCCTGCC
CCGTGTCCCTCCATAAATTCACCTGTGCCCTTGGTAGATGAAGGAATGAGGAGAAGGTTGGAGATGAACACTTGTGTTCTTAGAATAATCTCCT
TCCATTGCTCTGGAATGCCCCACCCCTGCCCTGCCCAGTATTGAGAGCCAGGGCTTCATCCCAATGCTTAACTGGAGAAGAAAGGGCACCTTCC
TGCCACTACACAGTTCGCCTCTTCTCACCCCTCTGCCAGAAGGCTCCAGGCCAGGCACTGCTCCTGACCACTCAGGAGTTCCCACTTTGCCCTT
TCTGCCTTCCCTGGGCTGTCTTTGGTCTTTCTGCTTATCAGAGCTGCCCTTAAATGCACCCTGAATAAATCAAGACCCTCATAGGTGCCCATGT
GCTCTGCTACATTAATGATGGTGACAATGAATGCATGAGCCCTGCTACATTTTAGTAAAGGACGGTGACATTAGGTAAACCTCCAAGTGGGGCA
GGGACTGGTGCCAGGCTTGTGGGACTGTTGCTGGGGGTCTCCAGAGCACCCTCCCCATTCGTTCCCATTGGACTCAGCCCAACAATGACTCCTC
CCGCACTGCCAAGAAGGGCCTTTCATGACCCTGAGTGCTTGGCAGGGTAGAGCTCCACTACAGTTCTCAGTAGAAGTTCACATCCACTGGTTGG
GCCGTTTGGGTTTTGCATGTCTTCTAATTTATCTGCAGACTCTGGGATGTTACTATTTTAGGCCTGCCATCTCTACCGAGTTTCCCCTTCACAC
TACACGCTCATCCCACTCCAGACGACAGAGCGAGGACTCCAGATGAGTTCGCCTTGCTTGATGAAGCTTCATTGCTATTGCTTGGTTCACAAAT
AGAAGTTAAAAGTGATCCCCGTTGGCCATTTAGAGGAGAAGGGTCCCAAAATACTTTGGGAAAAAAAAGGACAGGCCTATAATGCTCTGCCAGG
GGCCACCTCAGCACAGAGTGTTCCAAATGTGTTGGTTCTGCCTAATATACTGATTTAGAAATTCCACCACATGGAGGTTATTTGAGGCGATTGG
TAGCTGGAAGGTATTTAAAAGAAACTTGTTTCACCAAATTCTCATCAACCAAATTCTTTCTTTCATCTATCTGGAGAGAGAGAGAGACGGAGAA
GCTTCTTTTGTGCCAGACAGAGATCTATGAGCTTAGGGCATCTTCTAGTGAAAGGCCAATGTATAGGAAACATCATTTGTTATTTTAATATAAA
AACCTTTCTAATCCCTTTTTATAGATTGAATAACTCTCTCTCTTCAGAAGCCCTTATCTAAGATAATTAAAGAGCATATTTCTCATGAATATCC
TTGTTGGAGAAAAGAAAGTCCTTGAGGAGGAAATATGGAGGCTACAAACACTACCCAGGTTGTGAGTGGGCTTTTTCTTTCTTGTCTCAGGGAT
CTGTAAGATCGAGAAGTTTAACATGGGTCAGGTGTGGTGGCTCACGCCTGTAATCTCAACATTTGGGAGGTCGAGGTGGAAAGAATCACTTAAA
CTCAGGAGTTCAAGATCAGCCTAGGCAACATGGGGAGAGTCAATCTCTACATCAAATAAAAAATTAGCCAGGCATGGTGGCACGGCCTGTGGAA
TCCCAACTATTTGGGAGCCTTAGGCACAGGAATTGCTTGAGCCTGGGAGGTCAAAGCTGCACTGAGCCAACATTGCGCCACTTCACTCCAGCCT
GAGCGACAGAGTGAGGCCCTGTTTCAAAATAAAAATATTAACAGAGGCTTAACATGCCTCTTTGATAAAGTTTAAAATTCTTGTATTTGGCATA
TTCTAAAGAAAAGATGGTAAAATTAAGTGGGACAATTACTTTATCTGTCAAAATCCAACACACTCTTCAAAACCCGATTCAAGTCCTATCTGCC
TCAAGATATGTTTTTCTTCCTCCTTAACACTATAGTCTCCTCTCGCAGCCTCACCTCATTCACACGCTATTTATGCACCTAATTACAGATTACC
TTGTGGTCTTTAATTGTGAGTTTTCTCCTGGCTCTTTTCTTCCAGCCCTGTATATATTCACTAAATACCATTGAATATTTACTCAATCCTGCTC
CAATAAAGGAAGAACCAGAAGAAATTAGCTTTGGTTGACACAAGAAAAATTAACTTCTTATGTCCCATGAACTAAACTCTGAAAAAGAGTATCA
GCTAGAGTTCTGTAGGATGCGTTAGAATATAGAAAGCAATTTTTTTTTTTTTTTTTTTTGAGACAGAGTCTTGCCCTCTCGCCCAGGCTGGAGT
TCAGTGACGCTCACTGCAAGCTCCGCCTCCCGGCTTCACACCATTCTCCTGCCTCAGCCTCCCGAGTACCTCGGACTACAGGCACCCACCACCA
CGCCCAACTAATTTTTTGTATTTTTAGTAGAGACATGGTTTCACCATGTTAGTCAGGATGGTCTTQATCTCCTCACCTCGTGATCTGCCTGCCT
CTGCCTCCCAAAGTGCTGGGATTACAGGCGTGAGCCACTGCGCCCAGCCCTAGAAACCATTTTCATATGCATGGTGTCATTTATTCTCAAAAGA
TCCCTGTGAGGATCCACTGTGCTATTTGCTTTTCCAAACTCTTAAATTTAGCAATAATCACACCACTCTCTCCCGCCCCTGCCAAGTATCTACC
TTTTACAGAGAGCTTCAATAAGAGGTTCAGAGAGGTTAAGTCATTTGCCTCAGACCACACAGCTCATACTGACAGAGATGAGAGGCAAAAGCAA
GGAGTTCTGACCCCCCAGCCCAGAGTTCTTTCCTTTGCAATGAGCCATTTCCCTGTTTGGIATGGATTCACGAGAATTATGGAAGTTTCTTCCC
ATGAAGATCTTTAAGAGCCCTTAGTAACACCACCAACAGTCCTATGCTTGATAAATTTTAGGTATCACTCTTCCAAAGAGATGAAAATCATTCT
TGGTATCATTTTGAAAAATCTTTGAGCTTAGAAGTCTTCGGCCTTTTGGGCCTGTCTGCAAATAATTATCAGCCCTGCCCAATAGAAACTACAG
GAGGTTAGAGAGAGGCAGTTTTTTGCTATGAGTCATAATTTCAATTTCTATTAAAATTCTTACATGGCCACATATGCTACCTCTCAGGCTTTGG
GCCTCTGGGCCTGTGCTAATTTGCATGTCTGGTTTAGATAATGTCTGAGGTCCTTCCAGTCTAAAAAGGAGCCTCTGATGCTGTGATATAATAA
GTGAGATCACATTCCACCTACCAAGACTTGATGGTTCTTTTTCTTTTAACTACTGTGCTCTGTCTGATGTGTGGATGCACAGCTCAGGGACGGA
AGTGTTTCCTGCTCACCTGCCACGCCACAAAAGAAGGAGTGCTCTTGGCCGGGCGTGGTGGCTCACGCCTGTAGTCCCAGCACTTTGGGAGGCT
GAGGTGAGTGGATCACCTGAGGTCAGGAGTTCAAGACAAGCCTGGCCAACATCGTGAAACCCCGTCTCTACTAAAAATATAAAAATCAGCCAGG
CATGGTGGCAGGCGCCTATAATACCAGCTACTCAGGAGGCTGAGGCAGAAGAATTGCTTGAATCCGGGAGACGGAAGTTGCAGTGAGCCGAGAT
TGCGCCATTGCACTCCAGCCTGGGGGACAAGAGCAAGACTTCGTCTCAAAACAAAACAAACAAACAAACAAAAAGAAGTAGTGCTCCTTCAGCA
TGAACTCACCTGTCCACTTCTGCTACACTGAACCCAGAGACTTGTCCTTTCCTGCAAAGCATTTCACACATAGGTCAAAAGGCAAATCATGGGA
ATTGTTGGGATTTTACGTTTCTTGATTTGAATGGCCGTTCTAATTCAACGTGAATCCAAGACCACAGACTTGACAATCACCTGATTCAAAATAT
GTGAAAAGTACTTGAGTCTTTAAAATATGGGGTTCATCTCACCTTTTACTATTCCTCTCCCAAACATCAAAGAGATCCTGTCAAGTTTCTTAGC
TAACTTTGCTATCCTACAACATAAAGCAACTCTCATGCATAAAAAACTAATTAAAGGAATTGGCTTTCCAGAGGGAGCTTTGAGACCAATGTCA
CCCAATCTCTCACTTTTCAAAATAAATTATGGAACTATGTAGAGCTCTATTTTCATAATGTTAGATCAAGTCTTTCCTCAGAATTCTATATTTT
CAGAGAAGACCTCCCATCCCTGCACTGCCTACTTCCCTCCTCTGGTTTTCTCTTCTTCATAGCATTCATCGCCTTCTAATATACAGTGTAATTT
ACTACTTGTCTTGATGTCAGCTCCACAGAGGAAGGACGTTTGTCTAGCGATTGTTGTGTTCTCAGCATGTAAAGTGTCTCGCAAAGGTAGTAGA
CATTCACTAAACATTGTTGAATGAATAATGTGTGGTCCGACATAAGGCATCAATTAGAATCCAATAAGTTGGTCATGCCGAGGGGCAAGAAATG
AATTTTTGAGAGAAACAAAGGTGCTTAAGATACAACCCCACTTCCTAAGATGATCACCTAGCGAGCGACACAAGCGTACAAGGAAATTACACTC
AAAGGCTAAGCAGGACACGGGCAGTAGACACAAGACAATTCACCAGTGAGCCTTGACCTGCAGAACTTTGAAAGCAAAGGACACAGAAGCTGAA
TGTTACGCACACCCAAGAGTGTATCCCCTATGGCCAAGCACAAGAAGGGCAACCTCCCTGTGAACATTGCAACCTATGAGGGTCGGAGCACGGC
AGAGGGCGTTGCGTATAAACTGCCTCTCATTTATCCTCAAGCCTGATTACCTAAGTGAACTGTTATGTTTTCTTCTTTCTACCTGGAATCCTCT
TAAACCAGAGGCAGATTTGACCTAACCTATGCAATTTACTTCCGTCTACATGACCCAATATGGAGGAGTTTTGGCAAAAAAAACCCAGTTAAT
CTAGTCAGTATGAATTCCTATTCCACATGAGGCTGGCTCAAAATCCCACTTTGAGGCCTTATCAAAATAAATAGGTACATTTAGACATCTGTGT
CACTTAGACACAdAGCCTCTGGGAGAAATGAAATAGAAGAAGGTCCCAGGTTCAGCAGGGAGCCTCACGTGGCCATGGCAAGGCTGTGCCCAGA
GAATGTGCCACTTCACTCTCCACGCTTTTCATATTTAAAAGTCAGCTATTTCTTCCAAGCCTTTTCCTCGTAGCTCTTTTTTTCCCTTTGCCCA
TCTAATATCTTCAACAGTACCTCCATTATGCTCCACACAGCAGGGTCAGCTCCGAGGCAGAGGGGCTGTTGGATTCCAGGACAAGATCCTTGAG
CAAACGAAACCTGATCCAGGATGTTTTTTCCCTGTGAGTTCCAAGTTTGGGGAAAGCGTTCCCTGGATATTTTCCTCCTTCCAAGGTCTCAGTG
GATGCCAAGCTCCTCTGTTGCTCTTTGGGATGTAAGACGCCGCTGAAGTGAAGGTTTAATAGCCCCTCTAGAATGGTGGGATGTAGTGAGGACT
TGGTCCTGAGCTTCAAGGCCAGGTGGCCAGTGACTCTGGCACCTGACAGTGGGGGAGGAAGGAGGATGCTGGGTAATGCCTGGGCCTAACAGCC
AGGCAGACCACCTGTAGCCATCAGCCCTCTCTGTCTTCTCGGATGGGGTTTCTCAACCAGGGTCCAGGATAATAATCCACCTCTTCTTTTAAAA
AGAAAGCCACGATTTATTTTATTATATTATTATTATTATTATTATTTTTTTTTTTTTTTTGCTTTGGAGACAGGATCTCACTTTGTCACCCAGG
CTGGAGTACAATAGTGTATCATAGCTCACTGCAGCCTTAACTCCTGGCCTCAAGTGTTCCTCCCACCTTGGCCTCCCATAGAATGCTGGGATTA
CAGCCCTCAGCCACCACACCCAGCCTGAGACCCAAGCTTTATTTAAATTTGGGAATCACTCTTCCAGGAGCAAACCGTATAAACTGTCCTTCAT
CCTTTATAGCCCTCAGGCTTCTCTTTCTTTTCAAGGCCTTCCTCAATGGGCTTGGTAATCTCATCTCACCAAGATAAGCATGGTAGAGGGGCAA
GCAGGATGGAATACACTCCATGTATATGGTGTGTATTTTTAAAATCACCATTTATCACATGCTTATTTAATCCTCATAGCAACCGTAGAGAGCA
CCTGTTATTAATGCTTTTCAGAGAAGCAGAACTGGAGGTTCAAAGCAGCTCAGCAACATGACCAAGGTCACTCCACAAGGCCAATGGCGGAGCT
GGGATTTGAATCCCGGGTTAACTGATTCGAAGTCCTATCTCCTAACTGCCAAAATAATCCCCAGGCCCACCAGAGAGAAAGGATACCTTCGTGA
TCACATCCTAAGCCCTGCATCTACTTTCCCTCAGGGTGGGGCTCAGAGCGCGGACTCCACCAAAGACTGCAGCACATGAGAAGCTGGTCCTGAG
CTGTGGGTTGGGAAGGTTCGGGCAGTGCAGCAGTTCTCACACGTGGAGAGCAGGATCAAGGAACGCTTAGACGAGAGCAGCTTCTGCAGGTTGG
TGGCTGGGAGAGCCTGCTCCTCCCCAAAGACTGGACCTATTAGAAGCACAGGACCTCTGATGGAGATGGCCTTTTAGAGGATTTCCTAGTCAAG
CCCTCTACCAGTACACGTCATAACCTGTCGGACAGCCCAACAACAAACACCGCCGCTCACCTGCAGTTTCTTCCCTGTCGGAGTCTGGGAGTGA
ATCTTCCTCCTCGTGGGCTTGGCTCTCTTGGGAAGTTGAGGGCTTTGGGTCTCAGGTCTGCCATTGATTTGAGCATTTCTCCTGTGCATCCTCA
CTCTTTCAAGAAAGGACGTCTCGATCTGGCTACCAATCCCCCTGCTCTGCTGGGAGCATGAGCAGCCTTGCCCATTCCTCGGCTGAATAAATCT
GCATTCAGAAGCCAGGCTTTCCAAGGATAAACAGGGTTAGGTGCTGCCAGCCTTCTTAGCAGCAACCAGAAATTCAATGAGAAGTTTCAGGGAA
AATTCATCTTTTTCCACCCAGTCATTGAGAGCAAGGTTTTATGACACAGAGGATGAAGAGAGAGGCTTTTGTGGGCTGATGTCGCTGTGTTGCT
GGCCTCCGGTCAGGTCACAAGGTGCAGCGCTGCATGCCCGCAGGGACGTTTGCGCTTTCAGACAGCAGAGGCATCAGAGCAGAAGCAAATATCT
TCCCAAAGATGTTTGGCCATATTTCTAGGACCAGGTTAAAAGCTTCACACAGAAGCTAAGTTAACATTGGTATGTCTATGAGAGGGTCATGTCC
AGAAACAGCTTTCCCCTTCCAACACAGACTTTGCGGAATCTCCATGCTCATATTTCTCTACCTCAGGAGGCTCTCCTGGAATCTAATCTAAGAG
GCCATTTAGTCTCAAATTCACCCCCACTAAGCATCACATAATGGTCATACTGTTAAGGACATCATATATCTGAGCCTCAATTATCCACAGTGGC
TGGCAATGTCTTCATTCTCCAGACTGAGGCTCCGGCCTCTTTGCTTTGGCAAGAGTTGATCTCAGGACTGCCTGAAACCAGAAACTCCAGGCAA
GAAAAGTTAGTTTGTCAGAACCAGGGTGTGATTGTCTGCTGATTAAGCCATCTGCCACCACAGACAGACCAAGATCATCTAAACTGGTACACTG
GTGCCGACGGCCACATTTTTACACACTGGGCCTTTGGCTGCCACCTCAAGTTTGTCAGTTCTCAGGAGGCAGACTCCAGGCACCACATTCGAGA
GGGCCTCCATGCACCCTCAGGCTTCTTCTCATAGCAGGCACACCCTGCCTGAGCCCCACTCCATGCGCTGACGGAGAGTCGGGGCAGGAGCTCT
CATGCAGCCAGGTGTTGGGCTCTGCATGCGGGTTTGTGTGGGCATGTGAGCCACTGGGTCACCTCCCTGGTTCAGGCTTCTCCATTCAGGTTAT
AGACATATCTCTGTGTGTATGTTGGGGACTAGAGATGACCACACTGCTAAATTCTTCTTGAGTGACTTTTAGTAGGAAAAAAATATGAACCCTC
ACTTCAACAATGACATTGTTAAAAACACATAAATGCACACCACTTCACCGAGCAGCATTAAAGCCCTTGACTTCCCTGTGGGAAAAAGCTTTTA
GCCTATGCCTCATACATAAATAGGTGGATTGTGGTCCACTCAGACGGGACACAGGAGTGATGTAGTCTGTACCCCTCACACCCAAAGATGGAAA
TCTTTTGCCTGGAGGCTTAATTTCGTGAAGAAGTCAGGGTAGGAGCACTGGGTTCTCTTCAGCTAGTGATATCATGAACAATAAGAAGAATTGT
GTGTATGTAGCAAGTAGGAGAAATATTGGCdAAGTTTATTCATTCACAAATACTTAGAGTCTACTAACTCTGTGTGGGGCCCWGTTCTAAAAGT
GCTTGGATGTATCAGAGAACACAACAGAGATCCCTACTCTTATGGACCTGATGTTCAGGAGGGAGGGGGATATGAACCATAAACGACAAATACA
AATTAATTACTGATTAAATTATGTGATAAGTCAAAATGGAAGAGCACAGGAAGGAGGATGAGGGAGTTGCGGCGAGGAGGGATTAGATGTGGAT
TTGGAAGGCTGCCCAACATGTTTCTGGGTCTGAATATTTTTGGTCTGTGACACATCGTATTCTTGATTGCTLTACATCCACTGAGGCCACCAAC
TGTGCTGGGCACTATGCTGGGTGTTTACTTGGGTATCTCATTTACTCTCCAAGGCTGGCCTGCTTCTTCATACCCCTAAGGCAGTATGCAACAC
TTTAGTTCTTGGCCTTCTGCATCTTCATGCTTTTTCTTTGGAGCAGATTGCTCTGATGCTGGGTTTPACTTAAAAATCTTACATTGACAAAAAG
ACGTCTCCTCGAGCCCCAGAGCCTACGGAGTGCTTTTGTTAACCCCAGAGCGATGTGCTCAGGCTGTCCTTTGTGCAGCCAGGTACCAAAGTGG
GAAGATTTTCCATGCAAATACTTGGGAATAAAAGAAATGAGAGCGAGGCCTGTGGTCTTTGCTACTCTGACTCGACAGGCTGCTGGGATGAAGA
CGTTGGATGAAGACACCAGCTTAGGGCATGGCCTGTTGCTATTTATGACTCTATTACTTCACAGTGAGAGCACAGTTCAGGCTGACTGCTGGCT
GGCCTCTCTGTTTCTGTCACTTCCTTTTTCACCTATGTGAGCACTGTAGCATTCATTGGGACTTCCAAAGTCCTGTTAGCCTAGCTTCTGCCAT
CAGATGGGCCTGTGGGCACATTGCCCCATAAAACTCTGGCTCCTTCCTAAGGCCCTGGGGCACCACAGAAAGATGGGCCTGCCAAGCCACTGCT
CTGATAAGGCATGCCTTTAACCAGGCTCCATCACTCAGAGCTACAGGAAAATCCCATGGTCATCCGCCCTCTGTTGGTGACAGGGAATTCCATC
ATGCATTCAGATTTTTGCATGCGTGCTCTGTCCCTGGAACTAGAATAAGAGAATAGGGAGAAAAGCCGATAAGAAGTCAACGATAACTATAGCC
ATGGCCATAATTTGGGCTCCAAAAGAATAATTCTGAGTGGTTACCAGATAGCTTTCAGAGAGGGTTGAAGCCTTCAGTAGACTAAATGATTGAG
CAGAAAAAAATGTTTAGAAAACTTGAGACAGATTTTATCCATGCAGTAAGTCAGAAGTCAAGTCAGAAGTTCAGATGACATGTACGATTCCTGG
AATTTAGTCATTTTTTGTTTGAATGTGTTACTTCTCTTCTACTTTCTGTTCTGCTCCCACTAGAATTATCTCCTTCCCCCAGGCCCTCAGATCC
ACCCCACATACCACAAGCTCATTCTTCACCACACCCTGTTTTCTAGCATGGCTGCAAGCGCAAAACCTCCCTTAGTTCCCACCTGCAACGCCAA
GGTCCCAGAGTTTGCTGACCTACCATCAGCAAGACCTTGAACATGCCTCGGCCCTCTCTCCTCTCCCTCCACATTGCTCTACTTCAGTGGTACA
GCTCCTCCCATGGCTGTCTCTAGACCTCACCTTGACATCTCACCACCCACCTCTGCCTTGTTCCTACCTGATGCACTCTCCCCTCTCCTAAGAG
TCACCTGGCCCTAGTTCCACTCACCTCTCTGTCAACCTCCAGGAACCCTTTATGTGTCCTCCATGCCTTAGGACATGACCCGTGTTTCCTGAAC
AATCACACTTACCCTCTCATACTCACATGCAAGGACGAGGAACTTGTTTCCTATTACTTCACACCCACTCCCTGGCCATGCACAGAATACAGTG
CAGAAGTTGGTCCCAAATCTATTACCAGAGGCCTAAGCAGCAGCCTCCTCCTGCTCAAAGAAAAATGTTCTGCCGCTGCCAACATTGCACTTCC
TCTGCAACACATCTCACAAAATGCCCATCACAGCCTTCCACCTCATTCCAAGCTTGCAAGAAAGCTCGTCTTTCATTTGTCTTGTTTGTTTTTA
AGCAAGAAGAATCCCTTTAGAGGAGGAATTAGGAAAGAAAAAAAAGTCAAACAGAAACAGAAGGAGTGGAGGTTTGTCTCTCTCTTTTGTCCCC
TGCCTTGCCCTTCCTCCCTATCCCATCCCCCTCACTTTCTGTGTTGCTTTTCATGCCAGTTGCTAAAGACTGTAATTTTAAAACTGAACGGGCT
TTTCCAGAGCCTTGAGGAATGTGCATCTGCACGTGGATTTTTCCACAAAAAAAGTGTCCCTGTCTTTTGGATCTTCTTAGCCAGAAACAATGAG
GCCAGGCAATCAGAGTCCATCCGTCTTCCCAACTCTTATTCTAATTTGTTTGAGAGGCTCAGAAGGGGCTGAAGGGCATGGTTGTGTTTCACCC
CGTCAGACTCTGTTGTTTGATGAGGTTTGTGCTCCATTTGTTCGGATGGTTTTTATGGTTTCTTTGTTGTTCATGGAATAACTTTGAAGGTCAT
AATTCTGTATCCTCAAGCTCCTCTGTTATTCCTGCTCAATGTCTCAATGGGACTCATACCAAGAGAAAGGGAAGGAAGAAGTTTCCAGCTAGTT
AGTTAAGCAATTTCTCAACACATTTATGGAAGCATGTGGAATCTTTCAAGAAAGGGGTTACGATCACAAAAGTTCGATTTGAGGAGTATTCTCC
AAGACAACTGAGAAGCAAGCCCTTAGTCATGCCCAGGTGGGGAGCAGGTCTCCAACCTCTCATTTCCTGATCCAGCCCCTTTCCCAGCCACATT
TCTGGCCTCTTCCCTTTCTCTGTGGTCATCAGCCCAGCCCTCTCCACTATCAATAGATAGCCCGAGAGCCCTCAGAGAGCCTGGAAATGTGCTC
CCACCGCTGTGATGTCATCCCACATCATCCATGTGCTGAAATCAAGGACTAGAATCCGCCCCCAAGTATCTCATCGCCATCCGCTTTGAGACAC
TCTTCTTATGGGCCTTTCACCCAATCCAGACCTTGCCTGCTCTTCAGAGCTGCTTGAAGGATTTTGCTACAGAACTATGGACATTTAAGAAATG
TCTAACCTTGTGTCAGTTTATATTTGTAGAACGCTTCATAAGGTGCTTTTCCATATGTTATCTAATCTGATCATTTCAACAACCCCATAAGGTA
GATAAAACATAAATTTTTATTTCCGTATTAATTGAAGCCCAGAGGAGACTTTTGATTAATCCCAGGTTTCACAACAAGAGTGTCAGGAAAAAGA
GAACTTGAACCAATCCTCTGACTTCACATTCAGGGTTCTTCCCATTACTTTGGCTCCCAGATAAGACTACAATCTTCTGAAAGTCAGAGTGTTA
ATATTCCAACCCCAGGGGGTGAGTACACTTCCAGAATTTAGGGATGCTTTAAAGCCATTGCTGAAATTGPATTTTATTTCTCCTGATCACTACA
GATAATGCTCATGCAACTTTTCTGAGAAGCAGGCGATCCTAATGTACCCCTATTTGTTATTGTTCATTAGACCCTTCTTATGACTCAGTCAGAA
GAGGAGCTTGGGGCAATAACATGAATTCTGGCCTCAACAAAAGTAAGTAAATGTTTTATAGTTCTTTGGAGGAAAGCATGTTTCTGAGGACAGG
ATTGGAGAACAGGATCATGAGACACAGGAGAGCAGCAAGCATTTGTTTCCCTGCTTTTTGAGTAGACATCAAAATTATTTGCCAATTTAGGGCC
TTTCCATGATACCAGTCCTTGAAAGATGATATGGGATTTTCGTTTAGATTTTTGAGTTTTGTTTTATTCCGCTCGGTTTTAGAACATGGGAAAT
TTCAAGGTTTTCTTGAGACAACGTTCTGTACCAATGAATGGTCACTGTCACTGGGTTTTGCTTTTGTTATTTAATCAAGATAAATGACTCGCTA
TAATTTTGCTCCCGTAACTGTTGAGGCTAAGTCGTACCAATTTCCCCATTCCCCTTGATACTGGGTAGATTTCCTCATATCATCATCTCACCAC
TTGGTGGAGAAATTCATGTCTCAGAACCCTGCCATCTCCTGATAACGTTATCTTCTGTCCCTGATCAGGAGATGCCGGGGGGTCTGGGACATCA
GGTTCTCCACCAAGTGGTGAGATGATGACATGTCGAAGTCTACCCAATATCCCCCAGTAAGAGAATAAGGGGCAAGAATGACTCATGAACTCCT
GTACCAGAAATCAAACTTGCCATGCCTAGGAAGCAAAATATGAAGCACATGAACAGGTGGTAAAGGGAATTCTTTCAAGAAAGAGGTTGGGGTT
ACAAATTAATGCCCAAACAATAACCAAGACCCCTTCCAATTATGTGTATTAATTATTTACTTACTCTGCACATGACATTTGGCAATATAAGATT
CACAAAGATGAATCAGACATGCACTGGTCTCATAAGCGGGAGAGAGAAATCAAGCACACACAGAACTCCTTTCTTCTGGTTGGTATCAAGGAAG
GCTTTGTGGACCTCCTAGCACTTGAGTTAGCTCTCGAAGGAGAGGCAGGATTTGGACCTGTGGCGATATGGCCTGGGGATCACATGAGCTTGGC
ATGTCTGACAGTCAGTGAGGACCTCAGGTTCCTGGAGCTTAGGATGTGTGATGCAGAAGAAGTCTGGAAAGGTAAGAAACAGAATTGTAAATGA
TGCTATGAAGATATGCCTTAGTTCTTTAGGCAATGAAGAGTCACTAAGGTTTTGAACACTGGCGTGTATGGTGGGTTGTGGGGAGACAGAAGAG
TAATGAGGACACCAGTCAGACTAAGCCAGAAACCATGCAAGAAGCAATACAGATCTGTGATGCAGAAGTGATCATGGGAATGGAAGGGGTGTGT
GAGAGATACCAAGGAGCAAACTCACAGACAGGGACACCAGGAAGCAGCTGATCTTTGCTGTGATGTATAGTTTCTCAGTAAGGACTTCTCTAAC
TTTCCGATTTGAAAATGAAATCCACAGCCTCAGTACCATCAGTGATGCTATACTGTTAGTTTACTTTTAAAAATTTGAAAATTGAAAAGGTGAC
ATTTAAGCAAAGACAAAATTTTAAGAATTTAAAAATGTGTTTATTATTGTCTCTCCCATTGGAATGCGAGTTCTACCAGGAACTGCTTGTTCAT
TCCTGAATCTCCAGCCTGGAGAACAGGGCCAGCACATAGTAGATTCTAGGTAAATGTTTCTAAAATAAGATGAATAAATGAGTCAGTGGAGAGT
GGGCGAACGAATGAGTGAGAAAGCACATCTGTCAAGACGTCTTGCTCCCCTCGGGGAGCTGGGTCCTACTCACTGCATTTCTTTCCCTCTTGCC
ACAGGTCCTCCCCTTGGAGGGGCACAAACGATCAGTAAGAATACAGAGCAACGGCCCCAGCCAGGTACCTGCCCAGGATATGTAATCTCTCCTT
TGCTTCCTGCACAGTTGTTGATTTCACATGGTCAACTGATGGAGGGTTTAAACAATATACCACATATGTTTTCTCTTATTTCTCTCAAAGAGGG
TCTATCTTTGACCCTAATAGTCCTGTGAATGAAGTAACAGGTACTGACATCAAGTAGAGACTTGTTAAATATCAAAGGTGAAAGAACTTGAAGA
TGAACTATTCCAATGCTTTATTTTGTTCAATAAAGAAACTGAGGCCCGGGGAGGTGAAGTGACTTGCTAAAAACCATACAGCTGGTTAATGAAA
GAAATAGGGCTACAATCCCAGATCCCCCAAATTCCTGTCCAAGCCTTTTATGCTTCACCAAACCTTACTTTCAGAAATATCCCCATCCTGTGAT
GAGTTGGCTTAACAATTAAGGCACAGGAACTTTCAAAGCCATGGACCTGGAGACTCCGTATTGGAGAGGAACTTGGAGATTTCTGGGTCTATCC
TACCCTCACCTCTACCTTACTTGTGCTTGTAGTAAAACAGGGCGTTGACTTTCTAGCTGGGCTCAGTTCCACTATCTGACCCAGAGTCTGCCTC
CTGGAATTTCCACTCAAGCTTTTCCTTCTGGAGAACCCTTCCTCCTGCTTCCTCCGAAATCCTTTGCATATCTGTAGACAGCTGCTGTGCCCAA
GCCCCAGCTCCTCTCTCTATCTTCTTATGTGATGCAGCTGAGATCAAGTTCTGAACAAGTCATTTGCTTTCGGCTAGGCAAACATTGTTCCCCA
TCTCCACAGAAGGCAGAGCAAGAGAAAATCAACTGACATTTCACCTGGAAGGTTAAGAAAAAATTTGCTGACTGTGGATCATAAGTCAGATGTA
GAGAGTTTTAAAGACTGTCCAATAACAACAAAATTATGCATTAGTCATCACCTGCCAAAAATAATTATCATATTAATGATGTGCATGACAAAAG
ATGGTCTCAATGGGCTTAAGGACTTTTGAAGGTAATTTATGGTTCTAAGTAGAAACCCAAAAATCATAGTGATATTTTTAAAACTGCAATTATA
GTTGAAAAATAGCAAAATTTTACAAAATACATCACAATTTTGCCAACTCCAGACCCAGCTCTGTACCATGATTTCCTCAACTAACAATAAAGGA
GATAAACCTCTCACCTAIACCAGTATTGCAAAGACTTTTTCATGGCTAAATAAACACATACGTTTTGAGGAAAGTGAGATTTTAAGAATAGTCA
TTTTATAGCTAAAATGTACACTCTCGTTTGTGCTGACATTTCTGGCTTGTGTGACATTTCAAATCACAGAGTCTTATACTAGAAGAAAACTGAG
GAATCCAGTAGCCCAACCCACTCATTTGTCACATGAGAAAACTGATTCCCAGAAATTGTCCAACAAGTTATTTGTTTATCACATAGCTGCTGCT
AAAACCCAGGCTGCTTGGTTTCTAGGCATGTGTTTTTCATGCTACATTCAAAAAAMAACAGATTAATAATGTTGTTATGTATTAGGTAGAGTTA
TTAAAGACCAGTAACCATGAGACCTGCTTTAAGCCCATGGAGTCAGCTGTCCGTTGTATCATTGAATATTATCCAGTTCCTTTCCAAATGTTTT
CTTTGTTGCTTTGGGTAATGACAACATAATCTGCACATTTTAGATGCCAAAATAAGATATAAGTAATCCTGTTCCTATTCTTTGTTTCTTTCTG
TAATTATTGAGATGAAGACCACAAATGTCATCAAGATCTTTGAGATCTGCAAATAAAAGCAGTAAAGTTTTCTGAAATCAGTCTTTCCTGTGAT
CTTGATAAAAGTATAAACCCTTATGGTTTTCTTATCGTTGGTACGGTTGTCATATTTTTTIAAAACACTGAAGCAGTATTTTCCTTTTTTATTT
CCTTAGATCCGTATCAGATCCTGGCCCCGACCAGCAGTCGCCTAGCCAACCCTGGTGAGTTTACCTTGGCCTGCAAGCCTTTTTTGCCAGGATG
TTTCGTAGCTTTGTGAAATCACAGAGACTTCTGTCCTAACTCCAGACACCTGAGTGGGGAGTAACATGCACCTCTTTCCTTGAAATGTTCAAAG
TCTGTTTCTGGAGCATGTGAACAACAGGTCAGGGCTTGTTATCAGGTGTCAAGGTTGGTTAGCTGAGCATACTTCTAGGATAAACACATCAAAC
ACTGCACATGTGCACTTAGGTGTAGATGAGTAATAAGTGGCACAGGAAGGTAGGCGTTTCAATCAGAGAAAATGGGCAAGAAAGAGAAGAAGAC
AACTCCACTCAGCTCTCAGTTCTCTCCCACATGCCAGGCACAAGTTCATAAATATTCCAAAGGCTTTTATTTGTGATTTTAGAAAGTGTTGCCA
AGTTATCTGGCCTTATAAAAGAGATGTGTCCTGGAAAAGTTGCAGATATAGTCAATTTGACCTTGTACTTTAAAGATTTAGTATCAAAAATAAC
TCTCAAGAGGGTCCTTCTAAACCAAGTCCATCGAGATTGGGAGCTAGCAGGCCCTTGTCGCAGTGTGGCCCACTCACCTTCCCATCCCAGGGAC
TCTCCCTACCCATTCCTTACTGCCTGGTCATTACTGCCACACGACAAAGCAGCCACCCAGCATATTCTGGGTATTTTTCTCTCTGCACAAGTTC
TCTGTACAACTTAGAGCTGCAAGCCAAGCTCTGGGCCTGCCAAGGGCCAATCCAGGTCCTGTCCATCCAAAAGCTAAAGCTCATGGCGCAGCTT
CTCCCAATGACTCCCACAGTCATGCCTTGCCCCTGACTTTTTATTGCTGACAGGGAATTCCCTGGGGGACATGACCACTAATTAGAGATCAGTC
AGTGATTCAGGCCTTTCTAGATGAAGAGATWCAAAAGCCTTGCCAAAGGGATTGAGGGGGGATATGGCTCACCCAAGATCACACAGCTAGTTCT
GGGCAGAGTTTGCACCTCACTTCCCACATTCCATTTACCAGGCCTTGGGGCAAAGCATCTTAGGATCTAACCTCGCTTATTATACCCATCTCCA
AGGCAAGCAGAAAGGCAAATCAACAATGACATAAGAAGGGCTTGTCAAGTCGATCCCAATGTCGAAGGAAACAAAAGGTTTCTTTAAAAGGATG
AGAAGCTCCCTGCATTTAGGGAACTGGGTTCTGCCTTCTCTGGGCTGAGCTGTTCTGTTCTCTCCCGTTTGCCTCACCGCCTGCAGGAAGCGGG
CAGATCCAGCTGTGGCAATTCCTCCTGGAGCTGCTCTCCGACACCGCCAACGCCAGCTGTATCACCTGGGAGGGGACCAACGGGGAGTTAAAAA
TGACGGACCCCGATGAGGTGGCCAGGCGCTGGGGCGAGCGGAAAAGCAACCCCAACATGAATTACGACAAGCTGAGCCGGGCCCTCCGTTATTA
CTATGATAAAAACATTATGACCAAAGTGCACGGCAAAAGATATGCTTACAAATTTGACTTCCACGGCATTGCCCAGGCTCTGCAGCCACATCCG
ACCGAGTCGTCCATGTACAAGTACCCTTCTGACATCTCCTACATGCCTTCCTACCATGCCCACCAGCAGAAGGTGAACTTTGTCCCTCCCCATC
CATCCTCCATGCCTGTCACTTCCTCCAGCTTCTTTGGAGCCGCATCACAATACTGGACCTCCCCCACGGGGGGAATCTACCCCAACCCCAACGT
CCCCCGCCATCCTAACACCCACGTGCCTTCACACTTAGGCAGCTACTACTAGAACCTTACTCATCAGTGGCCTTCTAGCTGAAGCCCATCCTGC
ACACTTACTGGATGCTTTGGACTCAACAGGACATATGTGCCCTTGAAGCGAAGACAAAACTGGATGTTCTTTCTTGTTGGATAGAACCTTTGTA
TTTGTTCTTTAAAAACATTTTTTTTAATGTTGGTAACTTTTGCTTCCTCTACCTGAACAAAGAGATGAATAATTCCATGGGCCACTATGCCAGT
TTGAATTCTCAGTCTCCTAGCATCTTGTGAGTTGCATATTAAGATTACTGGAATGGTTAAGTCATGGTTCTGAGAAAGAAGCTGTACGTTTTCT
TTATGTTTTTATGACCAAAGCAGTTTCTTGTCAATACACCGGGTTCAGTATGACACAGAATCATGGACTTAACCCGTCATGTTCTGGTTTGAGA
TTTAGWGACAAATAGAGGTGGGAAGCTTATAATCTAATTTTAGGAGGACCAAATTCAGTGGATGGCAACTGGAACATTGATTGTAAGGCCAGTG
AAGTTTTCACCCAACTGGAATTTGATCGAAAGAAGGTTTGTGTGTTTAAGACGCCAAGGGCATTGCAGAATCCCTCTCAGTGGACAGTATGCAC
TCAGCTGACCACTCTCTCTAGAAATAGTCAAGATATGAACTAAGAAATTTTAATGCAAATACATACATTTCCTGAAAGACGGGAATTAAATTAC
TAATTTTTTTTTTTTTTTAAATGATGACAGTGGTCCCAGAACTTGGAAAAGTTGTAGGGATTTCTAAACTCAAGCAGATTCGCAAGTGCTGTGC
GCTTGTCAGACCATCAGACCAGGCCCAACCAATCAGAAGGCAACTTACTGTATAAATTATGCACAGTTATTTTCCTATATCTCACAGTATAAAA
AATAAATAATTAAAAATTAAGAATAAATAAACGAGTTGACCTCGGTCACATAAGCAGTTTTACTATCGAATCAATCGCTGTTATTTTTTTTAAT
GTAATTTGTACATCTTTTTTCAATCTGTACATTTGGCCTGTCTGTATGTTTTATATACCTGGTTTTTAAAAGCATAATATCCCTATAGCTGAAA
AGGAAACAGGGCTGTTTAAGTCACTGACTTATGAGAAAGCAAAGCACTGGTACAGTTATTTAACAGGCATACACAAGCAGGGAAAAGATAATCC
ATTTAGATCTTTAATGCTTTGGAAATGCGTGTAACAGTACTGCAATAATCACAGCTCTGGGAAAAACAACGAAACTTTCCCTTGTGGAGAGGAG
GGATTTTCCTCCTCTATATAAGCAACATATTTTTAGACATTAAAATATATATAATTTTGCAGGTAATTGTTGACTTTTTTAACTATATTAACTG
TTAAGCTGACAACTGTCAAAGAAGACCATGTTGTAAATAATTTGACTAAATAATATGGTTCCTTCTCTCAGTGCTGAGGACAGTTTTCTTATTT
ACCGCCCCCGTTAGGTCAAAGGGTTTTCCCTGGGGAACTTTCCTATTTACTTCTTGCACTATCAAGAATTTTTCGAATGTACCTACTGCAGTAC
AGCAGAAGGTAAAAAATCAGTGTGGTTTTTCATTGTTGTTGATGATGTTTGTAGTGTTTTTGTCTCTGTTATTTAAATCTTCCTCCAGCCTAAA
AGGCTTTTATAAAACAGCAGCTAAGGCCATGGATAAACCTGTATGTAAGGACTCGAGCAAGGCGAGCTGGTCTATCCAGACTTGTCTGTGAGAT
TTAACTCTGCAGCCTCCCCTGGGCACTTCAGACCCAGACGGCCACCTTCTGCCACTCCAGCAAAGAATAAGCGCCCTGCTTCCTTCAGGTCTCA
GACCAGCACTTTATGGCTCATGCAGATTTTTAAGGTCATTTTTCTTCCCAAGCAAGAAACTTGCCTCCAGTTCCTTCACTGTTAGGTAGCTTAT
TTTCATTTTCTCTATTTTACAATGAAAAGAGTGAGACCTGGGAAGTCCTTGATTTGCAAGGAATTAGACTCACAGCATTGGTAACCCTAGAACC
TTCTTAGGGTAACACTAAGTACCTTCTAGACAACATGTCTACCTAAATGAAATGGGATGTGTTTCGGAACATTTGTCTCCAGTTTTTTTTTAAT
CTTGCACCCTGCCATTTAAAAAGATGTGTAAAGCACATATTCTCAACATATGCACATTGATTTATAAATCATATATACATACTGTTACATTATT
CTTCATATTAGAAAACAAATACAAAATACAACATTTTAATGGTGATATAATATAAAAATTGAAACTGAAATTCTACTCTTTTCCTCCTATTTTC
TCTATATTGAATATCCTCTCCTATTAACTCTCAACAAGATGTTGGTCTTTAGTGTAAAAAAAAGTGTATGCATGTACTGGATTTCTAGTGACTT
CCAAGAAAGGAGAAAATTAAAACTTGGTATTAGTGGCCCAAGCATAGAAAGAGGAATGATGAAGGCCAGGTATGTTTAAGAACTAACTAGCTGA
ATTCTTTGGCAGATCTCCATCTTAGCCACGCAATTTCCCCATCAATTTTGAAATCTGCAAAGTATTCATTACCGAAAATGGAGGCATATATTTC
TGAGTGCTGTAAGAAGATGCTCAGCCCATTAGTTCATTCTTTTAAAGGTTTICTTTATGTGTCCTACTCAAACACTGGCTCAGGCTCATCTTCT
CTACACAGTCATAGAGCTTTAGCCAAATCTCGCCCCCTGAATATGTTCCACAGTGATTTGTCCTATGCCTCAGAAGCTGGCCCACTCGCTGACA
GGACTGGGGCACCATTTTCTCCTCATAAGCATTTGTACAGAGCGGGCACTGGCATTTATTCCCCTGCAATGCCCACATTGAACGTTTGCTAGCT
GGCTCCAGGGCATGAAGCGGACTAGTCCCAGAGGACAAACAGAATCTTTGCCCTCCTGTCTGCTAGTCQAGTCTTTTCCCTGCTGGATCATTGT
GTGCCTGTGCCATGAGGTTTCTGCCATCTAACAAACAGTGCCAGCCAAGCAGCGGGCATGCAGGCCTGCAAAGTGGTTCTCATGGAAAGCTGTT
CCTCCCCACAGTTTGGGACACTGACCAACAGTCCTTTAGCAGGAGGTCGTCTTAGTCCATTTGGTCTGCTATTCATAACTACCATAGACTAAGT
GGCTCATAAAAACAGGACTTTATTTCTCACAGTTCTGGAGGCTGGGAAGTCCAAGATCAAGGTGCCAGCAGATTCAGTGTCTGGTGAGGGCCCT
TGCCTTGGTTTATAGAGGGAACCTTCTCACTGTGTGCTGATATGGTGTAAGGAGCAAGGGAATGCTCTGGGGTCTCTTTCATAAGGGAATTAAT
CCCACTCTTGAGTGCTCCACCCTCATGCCCTAATCACCTCCAAATACCTTCACATTGGGGATTAGGTTTCAGCATATGAATTTGGGAGAAGGGA
CAGACACAGTCAGTCTATAGCAGAGGTCATCTTTTTTCAGTGTCGAAAAATGTTGTTGAGTCCCACACAAAGGAAGTTCTATTTTATTTTATTT
TATTTTATTTTATTTTATTTTATTTTATTTTATTTTATTTCTGGAGATAGGGTATCTCTATATTGCCCAACCACCTCTCAACATCCTGCCTTCA
AGCAATCCTTCCACCTCAACCTCCCAAAGTGTTGGGATTACTGGCGTGAGCCACCACACTGGGCCTATTTTCAATATCTGTTATTTTAAAACAT
TTTTAATAGTCAACAGCCACTAGAAAACGTAATGACACTTAAAAAGATTGTTATTTTACTTAACCAATAACATCTGCATGCAAGTGAGCGATGA
ACATACACAATGGGAAATGGATTAAGAGGTTCTCAGATAAGGTTTGCTAGTTTAGACCAAATGAGATCAGGAGGTTCTAGAACACAAGGGAGGA
AAGCAGCATCTGTCCACCCCAAGCCAAGTTACTGCCAACCCAAGGCAATGCTTAGTGGATAAATAAAAAACTGAACAAAATGCAGTACTTCATG
TGAGAGGTGGTTTCTGTGGACTTGGATTCTTACCGTATTTCCTCTAATATCTGTTCATTACAAGCACCATGTAACCTGTTGCCTTTAAAAACTC
TTGAAAGGTGTCCTTTGGGGGAATGTGCTTGGGGGAAGCAACCCTTTAAGCATGGCTGTCTCCTGCCCAGCCCAACTCCCACACCTTGCTCAGA
AGTTCCTCCACCATCAACAGCACCACTGAGCTTGTGTCCAGAAGGCCCTCAGGAGGTCGAGGTAAGAGGATGCTTTGTATCAGTGGGGGCAGGG
ATGGGAGAGGTAATAAGAGCCGGGATGGGGGCCAGGGGAGTGACTGGGTTCTGGTGAGCATACAGAAGCTTTGCTGTTATTCAGGAAGGAATGC
AGGTTTTGCAGAAAGCACATTGGGCATTTCATCACGTAGAAAGCTACTGAAACTGACACTACACGCCTTCAGCCAGGGAGTCCCGCTAAGGGAT
TATGAAAGGCCGGCTGATTCCAAAACTACAAATCCCAGGCTCCCCCGGGGGATGGACACTTGTGAGAGGAGTGCCTTAGCCAGTTAGAAACTCC
AGGGTTTCAGCCAGTTGAGAAAGGATAGGGAGGGAAGTTTTAAAAGGAATGTGGGCCCACAAGCCTGGGATCTGTCTCTCTCTCTCAGTCTGAT
TATGAAAGGCCGGCTGATTCCAAAACTACAAATCCCAGGCTCCCCCGGGGATGGACACTTGTGAGAGGAAGTGGGTTAGCCAGTCAAAACCTCC
AGGGTTTCAGCCAGTTGAGAGAAGGATAGGGAGGGAAGTTTTAAAAGGAATGTGGGCCCACAAGCCTGGATCTGTCTCTCTCTCTCAGTCTGAT
GCGGCCTCCTGTCTAATTGTTTCAGTCTGTCCGAGCTGGTAAATCCCTAAGCACATAGTCAATTTACACTCAGCTTAAAAGCTCTAGAGGGAGA
CAGGAAAAATTTCCAGTGAGACCACTCACTCACCTACAACTGCTAGATTAACCTTCTGCATTTTAACTGAGTTAACTCATATGTTTTCTTAGTG
TGTACACGAGGTTTATGGATAATCATCGTTGTCTGCTAGTATTGCCTAGTATTTTTCAAGAACATTCATTAAGTCCTTGCTACCTGGTGGGCAC
CCTGTTTGGGGAGTTCAGTGCAGGGTCATTTTTGTTTTGTGGTCAGAAAGACATTTTCCAAATGGGTGCAAAGTAGAAATAAGTTGGGTTTTCC
AGAGAGCCTTTGATTTTCCCCATCCACGGCCCTCCCATTGCCCCACTTCGGCTTTTGCCCTTCCTTGGCTCTATGTGTGCACCTAAGAGCCAGG
GGAGCGTGTGGCCAAGCTGCAGGATGTTGCAATAAATGAGATTTTATAAAGGAATGCTTAGAAATGTGCTTTGACAGCAAATGTTGGATGCGAT
TTTTCAATCGGCTAAGAAATAGAGGTTGGGAGCTGGGATTTTATGCCCGGCAAGCTCGGAGCTTGATTTTTCCATCTATAAAATGAGGATGATT
TAATAACTATACAAGAGTATCACTGGAGTTAAGTAAGATAGTGTACATAAAAATGCTCTGTATGTTGAAAAGAACTAGAAACTGCAAAGATTTC
CTAGGTATTATTGCCAAGGTCTTACTGTCTTCATCGAGAGGCAACTCCTCTTTACCTTCAAAACTCTGTACAGGTCTCTGTTCCTCTCTGATCT
AAGATAGAGTGAAGCCACACTATTCTATTCTATTCTATTCTGTATTGTGAATATACATTGTGATTTTTTAAAAAACTGTAAAATCTTGAAATAG
TCACAAACTTCCAGAAAAGTTAGAACAGTACGACGAACAATTTTTAGTGAATCATTTAAGAGAAATTTGCCAACCATGCTTCATCAACCCCAAA
TGCTTAAGGGTATATTTCATTTAAACAACAACCTTTTCCTACATAGTCCAGTATAATCAAGAGAATCGAATAACTAATGCAAGCGGATTACTCC
TATCTAATCCCGACGCACCATGCACTCTTGGCTGATTGTCACCACTGCAGTCTTTTGGAGCAAAGGTTCCCATTCTAATAACACTGATTGTGTA
TAGTTGTCATCTACCATTAGTCTCCCTCGGTCTTTCAGGTCTGTGACAATTTGAATATGACAGGTCAGGTATTCTGTAGAATGTTCCCCAAATT
TGGGATTGCTGGTATCTCTTCGCGATTGGATTGAGCCAAGGCTTTTCTGAGAGGACCATCGCAGAAGTCATGCTGCCTTCCTCTCCTGTATTCC
ATTGCGTGGTACATCATTTCAACGTATCCCGTTATTGATGAAGTTCACTCTTTTGTTTAAGTGGTGCCTCCCAGCCTTCTCCTTTGAAAAGTTA
TTCCTTTACCCTTTTGTAATTAATAAGTATTTTGCGGAGGTTTACTTTCAAAATATGTAAATATCCCATTCAACATCAAATTCAAAGTATTCAT
TCATTTATTTATATCTCTAAGACTCATGGCTCTCTATTTTATTGAGTGGATATGATCTGTTCTTTAAAGATTACATTGTCCCAATGGAAGGATA
TTCACGCTGGCTTCTGTCTCCTTTGACATGTTCCCATCAGTCTTTAAGTGCTTCCTTACTTTCTCGCACAACAACTTATTTTGGAGTTTCTGCC
CTAGCCCTGGAATTAGTCATCTCCTCATGGATTCCTAGTTCTTTTTAGTGGAAAATGCTATTTAGAACCCAATACCTGGGGAATAGATGTGCCA
ATTGCTAATGGGGTGTCCCTGCTCCCAGGCTCCCTCAGTGTACAGAGCCAAGCAATAGACGTAGATACATACATACACATATATTATGTATGTA
TGTATGTGACTATATGCACTCACGTACATACATACATATACAGTCATGCCTTGCTTAATAACGGGGATACATTCTGGAAAATGTGTCCTTAGGT
GATTTTACGGTTGTGAATATCATAGAGTGTACTTCACACACCTAGATGGAACAGCCTATGACACACCTAGGCTACATGGTACAGCCTATTGCTC
CGAGGCTACAAACATGCACGGCATGTTACTCTACTGAATACTGTAGGTGACTGTAATACAATGGGAAGTATTTGTGTATCTAAACATGGAAAAG
GTACAGTAAAAATAGGATAAAAGTTAAAAAATGGTACACCTGTATAGGGCACTTACCATGAATGGAGCTTGCAGGACTGCAAATTAATCTAGGT
GAGTCAGTGAGTGAGTGGTGACTGAATGTGAAGGCCTAGGACATAACTGCACACCACTGCAGACTCTGTGAACATTGTACACTCAGGCTACGCT
AAATCTACAAAACCTTTTCCTTTCTTCAATAATAAATTAACCTTAGCTTACCGTAATTCTTTATAAAGTTCTAAATTTTTTAACTCTTTTACTC
TTTGGTAATAACATTTAACTTAAAACACAAACACATTGTACAGTATAAAAATATCTTCATATCCTTATTCTATACACTGTTTTTATTTAAAAAA
ATATTTCGTTTTACTCCTTGAAATTTTTGTTAGAAATGAAGACATGAATTCATATTTAGCCTATAACCTGCCTGAGGCTGTTTTACAGTTAACT
TTCTAAACATGTAAGTAGGCATTATCTACGTATATCTAAAATCTATCATTCTAAAATAATGATAAAAATCTATCATTTTAAAGTAATGATACAT
AACAGTAACATAGTTGTTTACTATCATTATCAAGAATTACGTACTCTACACCTGCTAGACTTTCATAGACCGTAAGGGAGTAGGTTTGTTAAAA
CATCAGCGTCACCACAGACATGAAGATTGCTTGCACTTCCACAGGGCATGAGGAACCGATGTCACTGGGTGATGGGAATTCTTCACCTCCTTTA
TAGCCTTGTGGCACCACCATTGCATATGGGGTCTTTCACGGACCTACATCTTTATGCAGTGAAAATGACTGTATATCCTTATACATTTATATCT
ACCCTGTTCAAGCTCTGATGCCCCAACCCAGGCAACGCCTCCACAGGCAAACCCTCCTCAACCAGCTGTAGCCCTGACAGCCAGGACAGGCTGC
ACGCCCTGCGTCCCATGCTCTCTTTACTCCACTTGGGCACTAACACTTCCCATCAGGACGCGCCACACGGAAGCCCTTCTCACCCGTTCAGGCG
CATCCTATAGCCCTTCCTTGTCAATCCTTCTGCATGCACCCGGTCTTCCATCTCTCCTGCACCCCTCACCTCCATTCAAACGTGTTCATCTCCC
TGCATTGGCCCTAAACTCCCATCCAGGCCACCCCACTGCATGGCCACCCTCCTCACCCCACCCCAGGCAGTGCCTCTGTGTGGATGTTGTTCTC
ACCCTGTTCAAGCTCTGATGCCCCAACCCAGGCAACGCCTCCACAGGCAACCCTCCTCAACAACCTGTAGCCCTCACAGCCAGGAAAAGGCTGC
ACGCCCTGCGTCCCATGCTCTCTTTACTCCACTTGGGCACTACACTTCCCATCAGGACGCGCCAACGGTAGAGCCCTTCTCACCCCGTTCAGGC
TCTGATATCACTACCCCCTGACACCTGCCTTGCTCTGCTGCACCCATGGCTCCTGGAGTCAAGACTTCAGTGGGAGGGGGAGGAAGAGCGAAGG
AAGGGGAAGGAAGAAGGGAAGATGCGTGCTTACACATTTACAACAGCACATATCTCATTGGGTGTGTGTATGTGTTGCCCATGCATGGCAAAAA
GGGACCACAAGATCCATTAAACTGTAGTTTTGCCTGCTTCCATATTCTCTCTCTCACAGACACACACACACAGACACACACACACACACACGCA
CACACCCTTGTGCCTTTTGAGATGCAGAATCTGGCAAGCAGTTAGAGAAAGGAGAGAAAGAGATGAAGGAGTAGTTAACCAGAGAGGCTAACAA
GATAATAATAAGGAACGAGGCACAGACAAGTGGAGAAAATGAAATGTTAAAATCTTGTTTTAGGTGTGCAGGGGAGAAATTGGAAGTGTGGCTG
GTAAGGATGAAAAAGCAATGTTCTGGGTTATAACGTTAATGTTTTTGATAGAGGCTGGGGTGAAGAATGCATGGCTTTTTAATTATTTTTGGTT
GTTGTGTTCTGTCAAGAGAGAGTAATACAAGACCCCAGGATCTATTTGTATCTGAAGTCTGTGTCCTCCATTAGACAATAAGGGAATGAAGGGC
AGAAACTTTTGTCTAACTCTTTCTTTATAAGCCAGTGCCTGGCTTCTAGAAAGATCCAGAACATGTCTATTTGTCAACTACACCTACTGGACAA
AACGTGTCCAAACCCAATACAGCTGCCCAGAAATTCTGAGGGAAGCATGAGCTGTCCGTTCTGCTCAGTTACTGTTAATTCCGGAAATACACAG
AGAAAATAAAATCACGGAAGATGCAGTTAAAGTATGGAGCTAGACCACTGTTAAAACTTGCCAGCAATTTTTACCAATCCAATCTTTGGTGTTT
AATTGGTTTCCCCAGAAGATGCAATATCTCTTTCCCCACTGATGATTATATTGCCCAGAGCCGCTGGCTTTCAGAAATGTTTAATCAAGCATTG
CATATATTCAAGCAATACTTTCCAGACACCTCAGCTTTTCTTGACAGCGTTACCTTCCTTCGCTCTTTCTTTGTCTCTTTTAGGAACATCAAAT
CATCCAGTGATTTTCTGCATAACTCAAAGTTTTGTCATTTAGAAAAAAAATCATAACAGAACAGATAACCATTAGGAAACCAATTTGCATCAGA
CATCCATTTGAGAAGTTTAAAATGTGGGGAATGCAAACTCTACCCATAGGGACTTAGTCTGTAGGGAAGAAATATGAACTTGCCACTAATTTCT
GAAAAGCAAAATTAATTCAGTTATAGCTAGATCTGCCTTCATAAGGATATTTCTGATGCTTCTCAGCCTGTTCCTTTTATACTGAATATTTACA
AACTTCAGTGATCACCTGATCAGAATCCAAGACACAAAACTCAAATGAATCCAATTTTTCAAGTGGCTTCAACCCAGCAACCAAATCATAGGGC
TGCCATTTGTGCTCAAAGCCAACACTCCAAGGTTTTGTGTAGATGAGAATGAAGAGGGAGAAGGCGAGAAAAGAAGCCATTTCCTGCCGGGGGC
GGTGGCTCATGCCTGGAATTGCAGCACTTTGGTAGGCTGAGGCGGGTGGATCACCTAAGGTCAGGAGTTCAAGACCAGCCTGGCCAACATGGTG
AAACCCTGTCTCTACTAAAAATACAAAAATTAGCTAAGCGTGGTGGTGCATGCCTGTAATCCCAGCTATTCAGGAGATTCAGGAGGCAAAGGCA
GGAGGATCGCTTGAATCCGGGAGGTGGAGGTTGCAGTGAGCCCAGATGGCACCATTGTATGCCAGCCTGGGCGACAGAGTGGACTTGGTCTCAA
AAAAAAATAAAATAAAATAAAAAATAAAGGTGTTTCCATCCCCACAAAGAGCTATGTCACCAAAGACACAGGCACCCCCTATGACGTCCTGCTC
ATTTACCAACCCATATTCCTAGAAGATCAAAGCCCATTACCTCTGGCGAAGTTTGTTTAATTCCGGGAGGGATATTATGCGCATTTGTGTGAGT
TTCCAGAAAACTCAAAAGAGCCATAAAAATCGTATTTACATACAACATTCATCTAGGTTAAATTTCTTTGCAAGAAGTGTCTAATTCTCAAGAG
ATCAGCTATTCAGCTTGCTGGGAGCTAATTACAAATGGTCCCTTAGCTTTGCTCAGTCAACTGGCCAAAAGGCAACGGAGAGGAAATTGGAGTT
AAGACAACTGCAGTTGGTCCCTGGAGGGAGCCTATTCTACTGCCAGCATCTGGCTAATCAGAATGGATACAGATGCACAAGTATCCTTTATGC
ATGAGGGCCTGCTGGTTTTCCTGGGCGAG
HUMAN SEQUENCE-mRNA
GAATTCCCAAACGTGCACAGGGGAGTGAGGGCAGGGCGCTCGCAGGGGGCACGCAGGGAGGGCCCAGGGCGCCAGGGAGGCCGCGCCGCGCTAA
TCCGAAGGGCCTGCGAGGTCAGGCTGTAACCGGGTCAATGTGTGGAATATTGGGGGGCTCGGCTGCAGACTTGGCCAAATGGACGGGACTATTA
AGGAGGCTCTGTCGGTGGTGAGCGACGACCAGTCCCTCTTTGACTCAGCGTACGGAGCGGCAGCCCATCTCCCCAAGGCCGACATGACTGCCTC
GGGGAGTCCTGACTACGGGCAGCCCCACAAGATCAACCCCCTCCCACCACAGCAGGAGTGGATCAATCAGCCACTGAGGGTCAACGTCAAGCGG
GAGTATGACCACATGAATGGATCCAGGGAGTCTCCGGTGGACTGCAGCGTTAGCAAATCCAGCAAGCTGGTGGGCGGAGGCGAGTCCAACCCCA
TGAACTACAACAGCTATATGGACGAGAAGAATGGCCCCCCTCCTCCCAACATGACCACCAACGAGAGGAGAGTCATCGTCCCCGCAGACCCCAC
ACTGTCGACACAGGAGCATGTGAGGCAATCGCTCGAGTGGGCCATAAAGGAGTACAGCTTGATGGAGATCGACACATCCTTTTTCCAGAACATG
GATGGCAAGGAACTGTGTAAAATGAACAAGGAGGACTTCCTCCGCGCCACCACCCTCTACAACACCGAAGTGCTGTTGTCACACCTCAGTTACC
TCAGGGAAAGTTCACTGCTGGCCTATAATACAACCTCCCACACCGACCAATCCTCACGATTGAGTGTCAAAGAAGACCCTTCTTATGACTCAGT
CAGAAGAGGAGCTTGGGGCAATAACATGAATTCTGGCCTCAACAAAAGTCCTCCCCTTGGAGGGGCACAAACGATCAGTAAGAATACACAGCAA
CGGCCCCAGCCAGATCCGTATCAGATCCTGGGCCCGACCAGCAGTCGCCTAGCCAACCCTGGAAGCGGGCACAATCCAGCTGTGCAATTCCTCC
TCGAGCTCCTCTCCGACAGCGCCAACGCCAGCTGTATCACCTGGGAGGGGACCAACGGGGACTTCAAAATGACGGACCCCGATGAGGTGGCCAG
GCGCTGGGGCGAGCGGAAAAGCAAGCCCAACATGAATTACGACAACCTCAGCCGGGCCCTCCGTTATTACTATGATAAAAACATTATGACCAAA
GTGCACGGCAAAAGATATGCTTACAAATTTGACTTCCACGGCATTGCCCAGGCTCTGCAGCCACATCCGACCGAGTCGTCCATGTACAAGTACC
CTTCTGACATCTCCTACATGCCTTCCTACCATGCCCACCAGCAGAAGGTGAACTTTGTCCCTCCCCATCCATCCTCCATGCCTGTCACTTCCTC
CAGCTTCTTTGGAGCCGCATCACAATACTGGACCTCCCCCACAGAGGGAATCTACCCCAACCCCAACGTCCCCCGCCATCCTAACACCCACGTG
CCTTCACACTTAGGCAGCTACTACTAGAAGCTTCTTCTAGCTGAAGCCCATCCTGCACACTTACTGGATGCTTTGGACTCAACAGGACATATGT
GGCCTTGAAGGGAAGACAAAACTGGATGTTCTTTCTTGTTGGATAGAACCTTTGTATTTGTTCTTTAAAAACATTTTTTTTAATGTTGGTAACT
TTTGCTTCCTCTACCTGAACAAAGAGATGAATAATTCCATGGGCCAGTATGCCAGTTTGAATTCTCAGTCTCCTAGCATCTTGTGAGTTGCATA
TTAAGATTACTGGAATGGTTAAGTCATGGTTCTGAGAAAGAACCTGTACGTTTTCTTTATGTTTTTATGACCAAAGCAGTTTCTTGTCAATACA
CGGGGTTCAGTATGACACAGAATCATGGACTTAACCCGTCATGTTCTGGTTTGAGATTTAGTGACAAATAGAGGTGGGAAGCTTATAATCTAAT
TTTAGGAGGACCAAATTCAGCGGATGGCAACTGGAACATTGATTGTAAGGCCAGTGAAGTTTTCACCCAACTGGAATTTGATGGAAAGAAGGTT
TGTGTGTTTAAGACGCCAAGGGCATTGCAGAATCCCTCTCAGTGGACAGTATGCACTCAGCTGACCACTCTCTCTAGAAATAGTCAAGATATGA
ACTAAGAAATTTTAATGCAAATACATACATTCCTGAAAGACGGGGAATTAAATTACTAATTTTTTTTTTTTAAATGATGACAGTGGTCCCAGAA
CTTGGAAAAGTTGTAGCGATTTCTAAACTCAAGCAGATTCGCAAGTGCTGTGCGCTTGTCAGACCATCAGACCAGGGCCAACCAATCAGAAGGC
AACTTACTGTATAAATTATGCAGAGTTATTTTCCTATATCTCACAGTATTAAAAAATAAATAATTAAAAATTAAGAATAAATAAACGAGTTGAC
CTCGGTCACAAAAGCAGTTTTACTATCGAATCAATCGCTGTTATTTTTTTTTAATGTAATTTGTACATCTTTTTTCAATCTGTACATTTGGGCT
GTCTTGTATGTTTTTATGCTCCTTTTTAAAAAGCATAATATGCCTATAGCTGAAAACGAAACAGGGCTGTTTAAGTCACTSACTTATGAGAAAG
CAAAGCACTGGTACAGTTATTTAACAGGCATACACAAGCAGGGAAAAGATAATCCATTTAGATCTTTAATGCTTTGGAAATGCGTGTAACAGTA
CTGCAATAATCACAGCTCTGGGAAAAACAACGAAACTTTCCCTTGTGGAGAGGAGGGATTTTCCTGCTCTATATAAGCAACATATTTTTAGACA
TTAAAATATATATAATTTTGCAGGTAATTGTTGACTTTTTTAACTATATTAAGTGTTAAGCTGACAACIGTCAAAGAAGACCATGTTGTAAAAT
AATTTGACTAAATAAATGGTTCCTTCTCTCAAAAAAAAAAAAA
HUMAN SEQUENCE-CODING
ATGGACGGGACTATTAAGGAGGCTCTGTCGGTGGTGAGCGACGACCAGTCCCTCTTTGACTCAGCGTACGGAGCGGCAGCCCATCTCCCCAAGG
CCGACATGACTGCCTCGGGGAGTCCTGACTACGGGCAGCCCCACAAGATCAACCCCCTCCCACCACAGCAGGAGTGGATCAATCAGCCAGTGAG
GGTCAACGTCAAGCGGGAGTATGACCACATGAATGGATCCAGGGAGTCTCCGGTGGACTGCAGCGTTAGCAAATGCAGCAAGCTGGTGGGCGGA
GGCGAGTCCAACCCCATGAACTACAACAGCTATATGGACGAGAAGAATGGCCCCCCTCCTCCCAACATGACCACCAACGAGAGGAGAGTCATCG
TCCCCGCAGACCCCACACTGTGGACACAGGAGCATGTGAGGCAATGGCTGGAGTGGGCCATAAAGGAGTACAGCTTGATGGAGATCGACACATC
CTTTTTCCAGAACATGGATGGCAAGGAACTGTGTAAAATGAACAAGGAGGACTTCCTCCGCGCCACCACCCTCTACAACACGGAAGTGCTGTTG
TCACACCTCAGTTACCTCAGGGAAAGTTCACTGCTGGCCTATAATACAACCTCCCACACCGACCAATCCTCACGATTGAGTGTCAAAGAAGACC
CTTCTTATGACTCAGTCAGAASAGGAGCTTGGGGCAATAACATGAATTCTGGCCTCAACAAAAGTCCTCCCCTTGGAGGGGCACAAACGATCAG
TAAGAATACAGAGCAACGGCCCCAGCCAGATCCGTATCAGATCCTGGGCCCGACCAGCAGTCGCCTAGCCAACCCTGGAAGCGGGCAGATCCAG
CTGTGGCAATTCCTCCTGGAGCTGCTCTCCGACAGCGCCAACGCCASCTGTATCACCTGGGAGGGGACCAACGGGGAGTTCAAAATGACGGACC
CCGATGAGGTGGCCAGGCGCTGGGGCGAGCGGAAAAGCAAGCCCAACATGAATTACGACAAGCTGAGCCGGGCCCTCCGTTATTACTATGATAA
AAACATTATGACCAAAGTGCACGGCAAAAGATATGCTTACAAATTTGACTTCCACGGCATTGCCCAGGCTCTGCAGCCACATCCGACCGAGTCG
TCCATGTACAAGTACCCTTCTGACATCTCCTACATGCCTTCCTACCATGCCCACCAGCAGAAGGTGAACTTTGTCCCTCCCCATCCATCCTCCA
TGCCTGTCACTTCCTCCAGCTTCTTTGGAGCCGCATCACAATACTGGACCTCCCCCACGGGGGGAATCTACCCCAACCCCAACGTCCCCCGCCA
TCCTAACACCCACGTGCCTTCACACTTAGGCAGCTACTACTAG
CTCTCTCCCTGGTGGTGGGCATATAAAATGCAGTAGCCACTTCAGAAAACGGTCTGACAGACATCACTCAAGAGCCTCAGACAAAAGGTTGTCA
TGTGAGCAAGGGAGTCCACTATTGCCTATATATTTCTCTACGTTGGTAACAGATATAGTATGTAAAAGTGTTTGTAGCAGTACTATAACACTAT
GCATAGGTAAAAAACAGCTCAATGTCTATCAACAAATAAGGAAAAAAAATGTGCTAAGTAATATGATGGAATAGTACTCAACTGCAGAAAAGAA
TGAAGTAGATGACATAGTGATGCAGTGTAGAACACAGATAAGGATCAACAGTATTATGCTAGGCTAAAAGTCACTTATTTGTTGTCTATAGGAT
AGAACCTGAATAGGTAAATCCAAAGAGAGAAATGCTAGAGTAGTGTTTCCAGGGGCTGAGAGAAGACTAAACTGAGTGTAGATGGATTATATGT
CTTCTGCTGCTGCTCCTTCCATGTCTGGCTGACGACAGATGTTTTGACAATGGATGAGAAGCTGATGAATAATTTTTTTAAAAAAATACTGAAC
TTTACGTTTTAAAAGGGTAACTGTTATATGAGTACTCTCAATTTTAAAGGAAAAAAAGCAATGTTTAAAAGTGGGCCAATGTTGGAAGTCAACT
GTGAGAAGTTAAGAGCAGATTCACAAATAAGCACTGGAATTGGGCCTTGATTTGGGGGAATATTAAGAATATAACTAGAAGACGTTGTAAGAAA
AGTTTGGATGAGTCAGAAAATAGGGAGACCGGCTAGAGAGATGTCTCAGTGTTTAAGAGCACTGATTGTTCTTCCAAAGGTCCTGAGTTCAAGG
TCCCAGCAACCACATGGTGCCTCACGACCATCTGTAATGAGAACCAATGCCCTCTTCTGGTGTTTCTGAAAAGAGTGACAGTGTACTTGCATAA
AATAAATAAATAAATAAATCTACTTAAGAAAATAGGGGGACAATTGTGTGTTAGCAAAATACAGTAGAGACTAGACTAAGGTTCATTAAAACAC
AGGCCAATAAAGATGAGGCCACATGTATGTGTTCAGGACACATATACTGTAATACGTAAAATACATTAAAAAGTTCAGACTTCTTCAGGCTCGT
GAAAAACACACTGTGAATGCTTTAACCCTCATTGTCTGATCGTAACCCATTCCTAGTCATCTCCCTTTCCTTTCCAGCTAGTTTCCATCTTACG
TCCGCGTGTGTACATGTGCAGCTTCTATATAGGCCGCCACAGCTGATTCCTGTTTGTCCTTTTCCCGATTTTTTATTTGGTATTTTCTTCATTT
ACATTTCAAATACTATCCCGAAAGTCCCCTGTACCCTTCCCTCCCCCTCCTCTACCCATCTACTCCCACTTCTTGGCCCTGGTGTTCACCTGTA
CTGGGGCATATAAAGTTTGCAAGACCAAGGGGCCTCTCTTCCCAATGATGGCCAACTAGGCCATCTTCTGCTGCATATGCAGCTAGTACTTGGA
TGGAAGGCCACAACTGACTCTTAAAGACTGTATGTAACACCAAATATTAGAGCAATTGTCTTCACTTAGTAGCATTTCTGAGGGTTTAAGTCCC
TTTCATTTGGATTCTCACTTTCCATGTGCAATGAATTTTTTACAGGTGAGCAAGAAAGTATGGAAGGAAGCCTAAGAAAGGCCGTTCAATGAGA
CTTTAAAAAATGATCGCCAATTTTAGAACGCATCTTCTCTTTCAGACTTGGAAAATTAACTTTTATTACTGCCCTATGCAAACTAAATTTAAAT
AAGCTTTGGATTATACCCCGTGCGTATGAGCGGACGTTATGACATTGATTGACAACGTCAGATCTACCCTCAACCCTCTGACAGTATGATTCAC
ACATACCTGGGATCAGAAAGAAGTGTTTTCCAGACTAGGCAAAGTCACTAGAGCTCTGGTGGTACTCAGCGTAGATTTCTTCCCCATAATTCCT
AGCTAAAGATGAAAGATACAGACAAATGACCCATCCTAGGAGAATTTACAAGGGTAGTGGTAAGCAGGTAGGTTTCAGAACAGCTCATGAAACA
ATTCTGCAGCAGGAGTCAACGAGTCCCAAAGAAGTGGCCTATATGGGACAGCAAGGAGAGACTCAAAAAAAAAAAAAAAGGAGGAAAAAAAAAC
CCTATGTGGGGCTCAGCAGTGGAGCACTGAGTGCTCTTTCAGAGATCCTGAGTTCAAATTCCCAGCAACCACATGGTGGCTCACAACCATCTG
TAATGAGATCTGATGCCCTCTTCTGGTGTGTCTGAAGACAGCTACAGTATACTCATATACATAAATCTTTTAAAAAAAAAGTCCTATGTAAAAG
GCATAATGGTAGATGTTGCAAGAAAGCAGTGTGTGTATTGGTATCTGGCAGGAGGCTCAGGAATGTTCACATCACAGTCGTTAGGAAAGGAAAG
CAAGGGACCCCTTATTGTAGGATATATGGACAATTTAATCATGGGGTTTTTTTCCCCCAATAATAAAATTTTCAGCAGGAAAAAAAAATAACAG
TAAACAGTAAGCTACGTGTAACATAAGGATGAAAGATTTCAAAACCCGTCTTTTACAAAATGCACAGCAACAGCTTCTTTTGTGACAGCATCAT
CAGTCCCCGTGGTTTGCAGCCGGATGTAACAAGCCCTTTTGAAGAGCTAAGAGGGCAGAAAGTTTCTGTTGCCAAATATATAAGAGAAGCAGGA
AGCACTTAGCCTCGGACCAGGAAGGGCATCCTTGCAGGAGACCCTGCAGGTCTGTGCAGTTCATTGCCCAATGCTTCCCTTAAATAAAGAGCCA
CAGACTTCAGGAGPGTGTTCCCCCCTGAGTGATTCTGCAAGAGTTGTTTGCCCTCCACCAACTGGGCTGTGCTGAAGAGTTTAGTTTCCCAGGG
AACAAGATCTGGGTCTAAGGCAAGGCAAGCAGCATCCTATGCCCAAACCAGTAACTCCGTTTTCTCAGCCTGCCAATCACATTGGAGCTTCCAG
ACCATCANNNNNNNNNNNNNNNNNNNNGCCTCAAGTCTCCAGTCCCCATCATGATAGTGAGACCCAGAGGAAAGGGAACACCTGTTGCCACCAT
TTTGAAGGGACAGTGGTGACCCATAAAGAGGGTGCTCTAAAGGAGACAGGACAATTCAAGGACTTGGATAAAATATCAAACTGACAGTGGTGAG
CAACTGTCAGGTTCTAGGACCAGTTTAATCCTGACTGGGCAGAGGATAGGGGCCATCCACACAACCCCTGGAGGGAGCGTTCTATAGAACCGGC
TGCCTTGGTCAGCTTAGGGCAACCAGCATTACGCAGCAGCAGGAGGGATATATAACCCAGGCTCACCTCTCCCAACCCCTACAGGGGTTCCTTC
CTGCCAAGGCTCTCTACACCTTTAGTTTACAGTGGAAATCAGAAGGCAGTAGGACTCAGGTGCAGACCATATAATTCAGAACAGGAAACAGGAA
ATGTGGAAGAAGGAGGGGCTAAGGGAGAGCACAAACATTGGCCAGCTGCAGCCCGGCCCCAATTGGGACTGCAGACTTGGGTAGATGGATGGTT
GCCACATCCTGCTCTCACCCTTTTGAGTGACTCTGAGATAGAGCGCCTCTGTGCAGTTCCACAGAGCAGGCACACAGGCTGAAATCATGAATAA
TGTATTTTACCTCCTTGCCGGTTTGCAAGTCTTGAAAGCTCACTCCTCTAAGCTGATGGCAGCCCTCATGCAAAGCAATCTGATTGCTGTGTCT
GGGGTTCAATACACACACACACACACACACACACACACACACACACACACAGGTGCACGTGCATACACACAAAGACAGAACAGTTTGCTTGCAA
CATTGTCATAGCTTAGTGAACAGTATAGCATTGTTCTGGCTCAAGAAGCCCTGGTTCTTCAAAGCTCCTACTTAGATGAAATTATTTGCATCAC
AAACAAAAATTGTTTTGCATTTTTTAGATAATGAAGGATCGTTTAAATATATAAATTAAGGGCTATTTAGAAAAAATAGGTGTCTTTGTGAGAC
TGGCTTAATTCACTTACCATGATCATATTCAGTTATGGTCATTTTAAATACAAATCATGTGTGTTCCAATGACGTGAGATTACAGTGAACTATC
TAGGGAAACTAACAGGAGTGGAGTAGGGACAGGAGAGGAAGAGAAACGATGGGTGATGGGCACATATGCTCAAAACCATAATGTACTGGGGCTG
GAGAGATAGCTCAGCGGTTAAGAGCACTGACTGCTCTTCCAGAGGTTCTGAGTTCAATTCCCAGCAAACACATGGTGGCCCACAACCATCTGTA
ATGGGATCGGATGCCCGCTTCTGGTGTGTCTGAAGACAGTGACAGTGTACTCACATACATAAAATAAATGCATCTTTTTTTAAAAAATCATTAT
ATACTTGCATGGAAATGACCCTATGTAACCGAGTTTCATTTAGTTGTTTTTCAATTGTTAGAGAAAGACTAAATTTTCTCTAAGATAGCCATAT
GAAGACAGTTGATCACTCTATGTCCCAGAAGCCCATCTGGAACTTAGCATTTTGAGCAAACCATAAATTTGATGAAATACCTCCTATTCAAGGC
GGCAAGCATTCATTTGCCTCACATTTCTAAGTCTGCCCCTGTGACTGCCTTAGCTCCTTCTTCCCAAATTCTCCAGTTACCTGGCCCTTAGGGG
TGGTAGCGAAATCTCCTTCCTGACAGCTTCCTTGGCCACGTGCTTCTGTGTGGAGTAGTGCCCAGGCCGCCAGCTGTTCATAGTGTTTGGGCAT
ATGCACACACAGGAACGACTTGGGCAAACACCCTTTGCCTTGTTTCGGATGTTCTTCTTGCAGACCCTTGCCCAGACATAGGGAGTGCCTGTGA
AGTGCTCCAGAATAAATCGGAATGTCCCACTGAAGCCAGACACCGTGGTCTTCATGTTAATGTGTAAGTAGAGTAGTGATCATTTATTTATTCT
TTGATTCCCTTTTGCAGAGTTTTTATTGCCCTGTCTGAGATATTGGAGTTTTCTGCTACAGTAAATTTAGCAGGATTTCTCCAAGGTGACTTTA
GCGCCAGTGAAGGACTTATTGTGCCTCTTCAAGTGGTTCTATCACTCTGCTTGTGCTAGGGTAAAATGAGGTGCCAGTGTGGATTCAGGACAAG
CAGACACAAGCAAGCTAGGAAACCTGGCACTCGATCCCCTCTGCGGTGCTTAGGCAAGCCTTCTCGGCTACTGAATTTTGAACTTTCACAAGAA
CAGATCGGCTGTTTGTTCAATGGTTTATTACTCCTGCCACAACAACGCATGGCATGAGGTTGGCGCGGCCTGTTTATTTCTTTGTGTATATGGA
TATGTGTTTGTGGGTGTACATGAATGGGTGGTATGTGTACACACGTGTGCATGGGGAGACATGCCCAGGGCTATTTAAATCTTCTGCCCTTTTC
CTTCACTTCCCTCCTGTTCCTTCTTAAGAACAAGCATTGCTTCACCTTTTCACTTAAATTTCTCCTCTAGAAAAAACTGTTATCATTCAACTCA
ACATGTTTTCCACTGAAACTTCTTCCATATTAATTTTTAGGTTTTCATGTTCTCGTTTTATTCGTACTTATTGGCCGCTATTTTCAAATTAATA
GAGTCCAATCTCATATTTCTGTATATTGCATCCCTGTTGCCCATCTATGCTTCTCCTCCAGACTATTTCTTCCTGTGCTTATTCGCTTAGTGAA
AGTACATTGCCAGGCTAGAAAGGTGGTTTGTTGGCTAACAGTGTTCTCTGCACAAGCCTGGGAACCCGAGCTTGGTTCCTCATATCCAAGAAAG
GAGAAAAGCAACTCTGAAAGTTGTCCTCTAACTTTTTCATGTGCACCGTAGCCCATGTGCACCCACACCCACAAATACACACCACACACACATA
CACAAAATAATAATAATAATAATTTTAAATATATCAACTGACTTACAAACATTTGTTTTTTTTTCCCGATGACATTTGTGATGTTAACGCATAA
TTTGATTCTAATTTAGTTGTAAGCATATTCTATTTTGACTCTTTGAAGTTTTTTGAAATTCAGTATCTAGCTGTGGTGGTGAGCTTTCCACATC
AACTTATCATAACCAAGAATCACTGGGAAAGAATATTTCAGTTGGAAAAATTATTTGGATCAGAGTGATCTGTGGTCAGGTCCACGAGAAAATG
TCTTGGTTGCTAATTGGTGTTGGGGGACTCACTGTGAGTAGCACCATTCACTCATCAGGGGACAGTGAGCTGTCCAAGGCAAGAGAAACAGAGA
AGACCTCAAGCAAGCAGGCCGCATGGCTATTTGTTCATGCCTGCTTGTGACTCTAGACATTATGTGATGAGCTGCCTTGAGAGTCTGCTTCAGT
TTCTCCTCAGTGACAGACGGTAACCTGGAATTGTAAGACAATAAACTCTTTCCTCCCATAAGCTGCTGCTTCTTGGATGCTTTCTTGCTACAAC
AGAAATTAAACCAAGGCATCAGCTTATATTCTGCCTGAGTGCGTATCCCAGGTGCCCAGAAGTGTTGGCTGTTGTACTCTGCACATTTCAATGG
AGTATCAGTCAGGTTATGATGTCCTTCCTAATTTTTTAGCAAATGTGGTTTATTTTATTTTTTTCAATTTTTATTATTAACTTTTATTAATTAT
TCCATTCATTTACCTCTCAAATGATATCCCCCTTCTTGGTTACCCCTCCCCAGCCCCATCCACCTTCTGTTTATTTAATTAATTAATTTAATTA
ATTAATTAACCAGTACAGAAAAAGTCCACTGAAAAATCCATGTCCTGGGCCCCAAAATACAAATAAAATCAAAGGAGTAGAGTCCTGACTGCTC
ACAGATGAAATGCTTGTGAGTTTTCAAGACCAGCCAAAGACATGCTATGCGAACCTGTTAAAACCCAAATGGGCTGGAGAGATTCAGCTCAGCG
GTTAAGAGCACTGACTGCTCTTCCAGAGGTCCCGAGTTCAATTTTTAGCACCAGTAAGGTGTGGCTCACAACCATCTCTAATGGGATCTGCTGC
CAGCTTGTGGTGTGTCTGAAGACAGCTACAGTGTACTCACATAAAATAAATAAATCTTTAAAAAAACAACAAACAACCCAAAACCAAAAAAAAA
CAAAACAGAACAAAAAACAAGTTATATTAAACTTAAACTCCTCCCCCCTCCCCAACTATCTACTGTTTCAATGTTCTGGAGCTTAATGTAAAAC
TCCAGGTGGCCAGTCACCTTCTTGATTAAATAAGGCAAAGGTAACCTCTCCCTCTGCTCCAGAGTCTTTTTCTGAATGTCTCATATGAATCATA
ATTTGGAGCTACCTCTCCGAATCACGGATTTCTGACACGGGGAAGGTTTCCTATGCAATTAATTTTCCTCATTCAACTTGAAGTTGAATCTCCT
TTACAACATCCTCTTTAGTGCCCACCCGTGCCTTCTGTAGTCACGGATACTGTCTTCTCTGGCGGTCATTTAAGTTTGTCGCCCCTTCAAAATA
TAATAAACTCTTCGTGTTATGTGGAAACGTTACCCTTGGAACTTTTAACCATCTCATTTCAGCTCTCCAAATTTTTTGAACGGCTTAACTTTTC
TTTGAGTTTTCTTACATTCCTTTTAATATGGTAGTTCAGTGAAAACCAGGGCTTTTAGGTATGGTTGAGGAGAAAGTTGTATTGTAGGTATGAC
GGAGGATACAGTCAGAGGTATCTGGAAGTGGTCAGATTGAATTGAGCCATGAGAGGAGAGGGCAGGGGAGAGAGGAAGAGGAAGAGAGGGGGAC
GGGGAGAGGGAGAGATCACAGGACCACGAGAGGAGAGTGTGTGTTGGGGGATAGGGGGTGGGGTACCAATGAGAGTCTGCAGAGATGGCCAGTT
TATATGGAAATGAGAAGCTGGGAGAAGGGAAATGAATCCCAGCCTCTGGGCTGGAGAGGTTTAAGGTAGGGGCGGGGTGAGAAGTTCTGGGAGG
AGCTACAGGTACAGAGTGAACCCAGTCCCTGGCCGTTTGTACTTCTCTAACGAGCTGAGAACATTACCCTTTAATAATGATCTCCTTTCTCCAA
AGTGCTGTCCAGAACAGACTACAATGCCCCTGTTAGGGCTTCGAGCCCTAATACGGGCAGTTTCCTCTTTGCTCTCGCTCTGGGTTGTATCCGC
TCTAAGTTCGGTAACATCCAATGTCCGACTTACAGTAAAAGACCACCAACTTAAAAAAAAAATCTTAAAAAAATATTTATGACTTTTGAGTGGT
GAGACTATACAGCGCCTGGTCTGGTAGAGGTTATCACCAAGTTCGTAAAACAAGTGAAGGGTTTTCGGATAGACCCATGGGCGTGTCCCTACAT
CATCTGAAGGAGGAAGCCACTCTACCCTATAGTTCTTCTCCGGTCGAACTGAGCCACAGAGCAATCAATGCCTTGCGTCTGGAAAGGCGTGGCT
GGAAACCAGGTGGCCGAGCAAACGCTGCTCCAGGCTTCCGAACCCAGTCTTCTCTACCCCGGAGCACTCCTGACTACCGACCGCCCTGCATTCC
CGCCCTGGTCCTCACCGACCGCAGGAACTTCACAAAGCATTTCAGAGTACAGCAGATGCGCCGACGGCTTACGACCGGCCGTGGAGTCTCCATC
TTGCCCACAGACCCAACAGTCACGCCTCTCTTTCAAGCCTTCTCGATTAATGGCTCTTTCCAGGACTCGCGCCACCAGCTCAACTGCCGGGGCT
GGGCCTGCGGCACCGCGCTCAGGCCAAGTCCGGGCGCCCCTGCGCACTAGCGCGGGCCGCCCACTGGCTCCGCCTCCTCGTCTCTTAACTTCCT
AACTCTTGCTGACACCTGTCTAGCCTAACCCAGAGCCAGGTCCCCACCACTGCTCCTCTGCCCTGGAGCTTTCCTCTGGAGAGCACTGTTGGAG
TAGGCGAGAAAAACCCAAGAATGCCAGCCCAGCCTCTTCGCCATTTGAGCATCTCAAATGCCACCTCCTTAGGATTTCCCTTGGTTACCTTGTC
TAGTCATTCCTCAGCATTTTTTTTTTTTTTTTTTTTTTTTTTAGATCCCATCCAGATTACAGTATAAATATCCCCCAGGATGACGTTGATTCAC
TGGTAGTAAGTTAGACTTAGGCAAGAGAAGTTCCGATGTTCTGTTAGTCTGGTTGACCACGGATAATGAAAATGTATAGTTTTATTTAAAAACT
AAAAGGCTACAAGAAAGGAATTTGGCCGTTTTCAGCACAGAAATTATTCCGAAGAGACAGACTTATCCTGATATGAATATCACACAATGTGCAC
ATGTAATAAAACATCCAATGGTATCTTGTAAATAAATACAATTATGTATCAGTTAAAATAAAAAATATAAATTCTATAAAGCAAGGATCTGTCT
GCTTCTCTGCTCGAAGGGTCCCATGGTCTGGGCCACTCATCAATAGCTATAGAATATATAGCCTGAACTTATGGCCTGGCCCTGTTAGTTCTGT
CCTTGCTCTCAGACGTGCAAGCCTGTCTCTAGAGTACTTTTCGCCTCCCACTGCTGTTAGTTGTCCCACAGTTTATAAAAGTGCTCCGGTGTAG
CCAGGACAAACCATCTTGGAGCATTTCATGATGATAGACACAGGAAGAATATGTCACTTCCATTATGTGAATTAAGTCTCATGATTTCTTTTGG
AGAACTGAATAACCACCCGTCCTAGTTTGCTTCTCTGTGATAAAACAAACACTGACCAAAAGCAATTTGGGGGAAGAAAGGGTTATTTGGCTTA
CAGGAGGGAAGACAAAGTAGGAACTCAAGGCTGGAACCGGAGGCAGAAGAAGCAGAAGCAGAAGAACTAAGAGTGTTTATCAGCTTCAGTAGTT
CTCTGGTTTGCTCATTCATAAAGACCCGCAGCTCACCTGCCCAGGGACAGCATTGCCATAGCAAGGAGGCAAAAAACAAAAAACAAAACAAAAC
AACAACAACAACAAAACCCCTCCTTCATCAATTAGCAACAGGAATATGCTCCACAGACATGCGCACAGGCCAATCTGCCGCAGCCAGTTTCTCA
ATTGAGGCTCCCTTTCCCGAGGTGTTTCTAGTTTGTGTTGCCTTGGTAAGAATTAACTAGCCCACAACCCCAGTCCATTTAGATCTCTTTGGAT
CCTGCCCATCTAAGATCTGTTCATTTGGCTGAATCTGATCAACAGTTACCAACAATCTCACCCCAAGACGCTCACACACACACACACACACACA
CACACACACACACACACAACTGTCGACTTTGAATTTGAAGATTTTATTTGCCTTTACTAGGTGCCTAGCAATAACAGGAAAAGCCCTGTTAGGG
TGATGGCTTTTTTGTTACAGATCTAAAAGAGTAAAACAAAACCCAGAAAATGTCTTTCTTCATTTTGCCCAAGACGACTGCCAGCATCTTGCCA
CCTGGAAGAAGTGGAATCTTCTTTACACGCATTGTAACTGCCCATATACAGTCGATAGGCGGCGACATCTGGGCTTATTGGTCTTGATGGGTGA
ATTATAGTACCCAAGCTTATTTTTGCTTACAAGTTAGCCTTACATGCTTTTTTTCTTATCCCTTTTTTGTTCATTCAGGGGACATATTCGTTAT
GCTTTCCCGAGGAACATTAAAATCCAAACGTTGAAAATGTCAAAGACTGTAACATTTGGGAACAATGGCTTTCACGGCCCTCTCACCTGTGTCC
CTCACACTGTTTTTTGTGATGGTGGGGATTAAACCTAGGAGGGCCCTTTGAAGCCCAAGGACAAGCTTGGAAAATGTTCCCAAGTTTGACAAGG
TCTACTTGCAGAGTATTTTCGAAGGAAAATACAGGACATCCTGAGTAGGCGGTGCGAGGTGTGGATGGAGCCGGCAACCAAGGAAGTAATCGGA
TATACTAGCAGTCCCCGGCATTATTATGCCATCCATTCTCTAAATCTACTTGGGTCAGGCCCATTCTGCGGTCTGTAAAGTGGGGAAATCCTCA
CTTTGAATCTCTTCGGTGTACCCTAGACGGAGAATACTAGCGGATCCCTTAAGGGCCGGGGCCGGACAAAGGGGTGCCGCGGGGCTCCAGGACG
CACCGGAAGGAAGTAGGACTTGCACATCAGCCAGGGACTATGCTGCTACGGTCCCTGCGTAGTTGAGCTGCCCGCTCCCTGCGTAGCGTGGGCC
CGGGGAGCTCCGGGAGCCCCGGGAGCCTCGATTCGGGCGCCGGGCCGCTGTGGGCACCTCGCCGCGCCPGGCCTCCGGGTAACTAGTGCTTTTG
TGCCACCTCGTAGGGGCCACACTGAACCCCGCCCCCTCACCTGCTAGTCCCCACGAGGGTCTGGGGACTGTAGACCCTAATGAAAACTGTCAGG
ATCGGCACTTAGTGGGGCTCAAACGCGGCTTCTCGGCCCATTTTTTTATTTTTATTTTTTTATTGTCAGATTCTTTAGGCTTGGGAGGTGGCTA
GTTGGGGCACGAGCTGTATTTAATCAGCTCTTAGTGGATGCAGGTGTCTGCGGACCTAGGGGGCGGGTTCAGGCTTGCGACTTTCTCCTCTTGC
TGGCAGTGCGGATCTTTCTCTTCGGAAATGGAAGCCAGGTGACATAGCTTTCATTGGCCTCGGGCTGCTGCTGCCAGGTGGGTTGGCAGTGTTG
CCAGTCTTGGCCTAGACAGCCTGGGTCCCAACCCTTGGAATCTCTCAGAGTGTAGGTTATCCCTGGGTGCGCTGTGAAGATCCTAAGCACACAA
TCCAGGTGAAACGGAAAGTAGAATGTACCGAGCAAAGGAAACTAAGCAAGCACACATGGGCCTGGGCCCAGTACAGCGACACATACGGACCCAA
CTGTAGAGAGTCCCAAGAATGCCAAAACATTTTTTGTAACCAGGGAACACGCATACGCAATGACTGTGTGGTCATTTATCACGTCAGCACAGAC
CAGAGTGAATAATTGAAACATCGATTTCCTTTCCTTTTTTAGATAAAGATAGAGAAAATGATAAGGAGAAAAAAGCAGTGGTAAGTTTCTCTTT
GCATGCTTTCTCCAGCGTAATTTCGGTTTGGTTTTGTTTTTTTTTTTTTTTTTTTTTTGAGGCGGAGAAAACAAAATGGTGCGCAAAAAGTGAA
AAACCAACCCAAGACGATATCCAGGGTCACATCTCTCTTCCTTCTTGTGGCCCAATTCCTCTCCTTAGATTCAATAAAACCGTTAGCCAAGTTC
TTGTGTGTACGGGCGGGAGTGGGGCCTCGAGCTGCATTCAGGTAGTAAGCACTCATGTTCAGGTGTTTGTGACTGCCAAAAGATGCTGACCGAG
CCATCTAACGGCTTATTATCTATTTGCTTAAAAAACTGCCGATTGCCCCGCCCAGATGTGGGGGGGAAAGTCTTCTTCTCACTTCCTTCTGCTG
TCTAGTGGAGACCAGAGAATCCACGACGGACCTCAGGGTGGCCTCAGTAAGGAGGTGCTTGACAGGCAGGCTCTGGGGGTTGGGGCTGTGAGGT
CAAGGAAGTCCAAGTTAGACAGGTTTAATGTTGCAGCAGTATTTTGGTTACCACCAGCCCAAAGGGGTTCAGTTTCTTCCTTTGACATAAATCT
CACCCCAGGCTGGCCTCAACTTGTGATCCTTCGGCTTCGACCCCTCCTGAACACTGGCATTTCAGTTGTGTGTCACTCCGAAAGTCGTCAGGGC
CCGTTTCTGTAAGGGAGCTGAAAGCCTTGAGCTCAGATCGCTGGGGACATAGGCAGCTGGAGCACCTTAGTGCCTCTGGAGGGTGGAGGTGTGC
CCTCCTGTACCCCTTGGTGGGGCAGTGGAGGTAGTCAGGAGGAGGCGTGTCCCTGGGAGCAGGAGTGACATCACAGGAAAAAAGGAGGCTTGGT
TTTCAGGCTGGAGATGGCTCAGCAGTTAGAGCTCTTGTTGATGTTAAGAAAAGATTAAAAAGGTTTTTGTTCCTAGCACTCACGTGACAGCCCA
CAAACTGTTTACTTCCAATTCCAAAGGATCTGACCCCGCCTCTGCTGCTCTTCATGGACACCAGGCATACACTGGAGCACAGACGTACTAGCAG
GCAAACCATTTATCCATATAAAATACATCTAAAAAGTTAGAGGAGGAAGGTGGTGGTGGCGGCACAGGCCTTTGATCCCAACACTCGGGAGGCA
GTTCTCTCAGTTGAAGGCCAGCCTGGTCTACAGAGGGATTTCCAGGACAGCCAGAGTTACACAAAGAAACCCTGTCTTAAACAACCAAAATAAA
TAAATAAAATGAATGAAGGAGAGAAAGAAAATATGGATAGCTAGTGACATAGAATTTGTATTTGTAATTGTTGAGCTAACCTTGCCAAGTGTTG
GACCTGTTCTGGATATGGTCTGATGCACCCCTGGGTGTACAGTAGCAGTTTGTAGAAAGTGGTAGTGTGGAGCCTGGTCTCAAACTCAGGGTAG
CCATTTAAGTACATTTTTGTGTGGGAGGCGCTGGGCTGGAACCATGAGACTTGAAAACAAAGCCATTGGCATTGCGCACGGTAACCTGACAGAC
GGGACAGTAAAAAGGACATCAGTGAATCTAGAAAGCAGTGAGTGAGCTTGGGGCTTTGAGGAGGGGACTCAGGACTCTGTGCAGCCTTGGGAAG
CCTTGATGCCCCCTGAGAGGTAGAGGAGAGGGGACTGTAGTTGTAAGAATCTAGAGAGAGCCGTAAACTGGCTTTATAGTCTGTCCTTAGATAG
TGGAGGCTTGGAAGTGTTCAGAGAAATTGGGCTAGATGCCAACGTCAGAGTCACCAAAGGACTTCACCATAAACTGTGTAATTTTTACTAATTT
ATTTATTTATTTTTTAAGTTTTCGAGACAGCGTCTCACTGTCTTACCACGTAGCCCTGTCTGAATTGGAAATCACAGATCCTCCTGCCTCCGCC
TCCCTAGTGCTGGCATTAAAGGGTGTATCATGTAACCTTTGAGGAGTAGAATTTATTTGTTTCGTGTTTCTGCGGGCTGGAAAGTTCAAAGGTT
TCCCTAGTGCTGGCATTAAAGGGTGTATCATGTAACCTTTGAGGAGTAGAAATTTATTTGTTCGTGTTTCTGGGGGCTGGAAAAGTTCAAGGTT
TTTTATCAGGACCCATCCACTGATAACTGCCCTACCTCACAATACTACTCCCGAAATTCGCCTCCACTAAGTCACTCTTTGTGCATGACTGCTC
TATTTGCAGGTACACCTGCATGCCAGAAGAGAGCATCAGATCCTATTACAGATGGTTGTGAGCCACCATGTGGTTGCTTGGAATTGAACTCAGG
ACCTCTAGAAGAGCAGCCAGTGCTCTTAACCACTGAGCTATCTCTCTAACCCCCACCAGTCACTCTTGATGGCGGAGTTCACACAATCTCTCTG
CAAGGTCTAATCTCTCAACACTGTTCTACCGGGGACTAAGTTTCCAGCTTGTGACTTTAGGGGATGGATTCACATCGTAGCACTGCGAACGCAC
TGTCGTCCGACTTGCCTTTCAGGGTGAGATGGTAGGGATCACACCAGGAGAGCTGTGGGGTAAGCTGAGAGGTACAGAGGAGTAGCGAGGTCTC
CCTCTAGGAGTAGGAGAAATGCTACTTGTTTGTCCTTGGTTTTCATTAAACATCCAAATTGATTGGCAGTTCTGTAGGGTTACGGTGATCATTA
CTATTGTATTGCTCCTCCCCTGACAGGGTCTGACTGTGTCCTGGCTGGCCTTGACTCACAGAGACCACCTATACAATGAGAAATGTTTAAACTT
CCTTTTTTTGATATATTAAATGTTACTTAAAAGAAAGGTGTACAGTTGGGTATGGTGGTACAGTCCTGTAGAAACCAGCATTGAGGAGACTCAG
GTGAGAGGTTGGAGAATTTGAAGTTATCCTGGGCAACAGCAAGACACTCACATACATCCCCCCCCCCAATAAAAGACTTATTTACTTTATTTTA
TATGAGTATACTGTAACTATCTTCAGACAGAGAGGACATCGGATACAATTACAGATGGCAGATGCTTGTGAGCCATCATGTGGTTGCTGAGAAA
TTGAACTCATGACCTCTGGAAGAGCAGCCAGTGCTCTTAACCACTGAGCTATCTCTCCAGCCCCCCTCTGTCTCTAAATAACTAAAAAGTAAAA
ATGAGAGTGGGPGGATTTCCGTCCTAACGCGTGCTTAGCATAGCATCAGGTGAGTTCCATTCCCAGCACCAGAGAAGATGCTCACCCCTGCCAG
TTACAGTGGCTGGAGAGTGGACTCTGCTTCTGGGGGAGAGTGGGCTGAGCCGCGGCAGAGACACAGAACTTTGAGCCAAGCTGCCTGAATCCTA
CAGCAGCACAGCCTCCTATCTCTGTAATCTGTTACTTTCTTTGCTACCCTCACATTTACTGTGTACACTGTTGAGGCTGGGACCAGATTTTCCA
TATGATGTGCTGGGCTCATAGTAGGCCTTCAAGCAGTTGAAAGACTCAAACCTGCCTGGTTTACTGCCTTGTCAAATCACCACGTATTGGTGGT
AACTTCTAGCGAGGGTGTGGAGCAACAGGCTACTTGCTATATGGTAGGGTAGGCACTTCTCTGTGTGACCCAGAGGATGTGCAAGAAGGTTCTG
AAGGAGCAATCTGTCTTAGATTGAACTGTGCACCTGTCGTGATCTGCTGGAGCCAAAACCAAAATATCGATTGAGGGCTTCTCTGTACAAGTCA
GCAAGTTGGATCCTAGACGGCATGCACGCGATTTTTAGTCTCAAAGCCATTGATAAAAGTCAGGTTAAGGAAATGGGACACTTTTATACTAACA
CAGACAGTGTGAGGGTGGATCTAGGGATAGGGCTCAGAACTTGACTCAAGCAGACATGTTGTGTGCAGTTTAGGTGACGTAGGTGGCAGGGAGA
AGGGTGTGTTGTTTGGGCTGAGGATGTAGTCCAGGGGCACATAGCTTGATTGCTATGTATCAGGCTCTCCGTTTGACCCCTAGCTTATTTAAAA
CAAGAACAAGCGGGACCGGGTCAGGTGTGGATGGATCACATGGGTTTGTCAGAGGGTGCGGCATGAGGCATGCTGATACTGTGTGGTGGGACCT
TGGGGGATTCCCAAGGTAGGGTTTTAGCTTACCTCTCAAAGTCCTGGCCCGAACCAAGAACCTGGCTATTCACCTGAGCCTAGTTCTGTCGTGC
TTTATCTCTATGCCTAATGTACATTTGTCTTCTGCCAGCTTTGTATTCAGGGCAATATTGATAGTGGGTAGACGACATGCCTGCAGTTCTTCTC
CAATACAACAGACGTCGAGGTAACGCTCTTACAGTGGGTTTGTAGGAACCCGAAAACACCTCAGTGGAGTAGTTATACCGGGCACAGAGAGCCC
TGTCCAGCAGAAGGCTGATGCGAGCTGCCAGTGAGCTGCACGTCTGATCCGGTGCCTTTAAAGCCTCGTGGAGTGAAGCAGGGGGAGCTGTAAG
ACAGCCCTGATGTCAATTAACATGTCAACATGAAGTCAGTGTTAAAGCAATGAATGGACTGTTCACATTCTGTGTTTGATACTCAATCCCTGGG
CCTGGAGTTCAGTTAGTGTTTACAGCTTTCTCAGTTTGGACTCCCAGATAATGCCCAAGGCATGATTTCTCAACTCCGGATTTTGTTTGACTAT
ACCGTGCTGCCAAGGAGTATACATTCAGTCCAGTAGATTAGTAATGCACTTCAGCTCGGCCTGTTTTCAAGTGAGGACACAGCCCCCTTAAAAG
AAGAGGCGCATCTGTGGTCAAGCTTCAAGGTCATCGTGGTTGCATGTGGCTTGTGGCCCCTGTATGCAGTGGTACATGAGGTCCCACCATTCCT
GTCTGAGAAGGCAACTTTCTGTGTGGGGGCTGCTGATGCACAGTATGGATAAATCCTGGACAAACTGCTTGCTTCTTGGGGAGCTGAACTTTTG
GAATGTGCCAGGCAGTGATAGTCTGTGTAGTTATGTGACCCCTGAGCTTCCAGGCCTAGGTQACCTTTCCTGGTAGACAGTACTTGACATGTGT
TGTCACAACTTGTTCCTGGGAAAATTGAGTGTGTCTTGTGTGACTCCATTGGGCAGGATACTTGGGAGTTCTGGCCCATCTCCCTCACAGGTAC
ATCCCCTGTGCAGATTTGGTGCTGTGACCTCTCATGGCAGTTAATATTCGCTGTACTGTTGGATGATGTCTCATCGATCATCACCGACCAAAGC
GACCGGCCTTGAGTGGTGACCCTCTGAACAGAACTTGAAAAGTGCTTATATCCTCACTGTAGACCTTGGGGCCAGTTAGCAACAGTAGCCGCAG
CAGTAGCTAAAATGATCCAAGGCACCGTGTTAAGTGTTTATGGCATATCTAATTGATTCCATGCTTATCTGCTTAACAACCCTCTGAGGTAAGC
ACTGTTATCTCTGCTGAGGCGAGGAAGCAAAGGATAACTGGCTTATAGAATTTATATGGTGTGATAGTTAATTTTAGGTGGCAGCTTGCCAGAA
TTAACGACTGCAGCAGCCTCCCCCACTCAGCGTGGTTTCAGTTACCCGAGGTCAACCAGGATCTAAAGTATTAAGTAGAAACTTTGGGATGTAT
TTAAGTTCTGCATTGTTGTACGATGTACCCAAAGAGATCTCATCCCGCCCATGTAAGCCATCCTGCTGTCTAGCATAGGAAAGAAAAGCATGGC
AAGTGTTAGGCTTGTTGTACCTGAAGCTTCAGGCAGCCGCTGGGGATCTTGCAACCCCAGGAACAAGGAGGCAGCACTGTGCCTGGGGGCTGAT
GAAGTGTTACCTCTATACACGTTTAGAGGGTGCTTCTGGAGAAGACCGATATGTAAATTGGCAGCCTTACTAATATATAATATAACAGGTCATA
TAATTGGCCACAGTCCTGGATAGAACAAAGGAGAAAAGGATTTTCCCCCCCTCTTCCTGCTGTGGGACACCAAAAAAACTGCTGGCTTTCCAGC
CTTAGGACTTAGGGACTTAGCCAGAGGCCCCAGGCTCCAGGGTTTCAGCCCCATACTTAACATACGCCAGTGATCTCTCTGTTATCAGACTATT
AGACCTAGACTGAGCTCTGCCGCTTGCATCCCTGGGACCCAGAGCTGGAGACGACCTGTCATGACACAGAGGTCTTGGCCTCGATATGGTAGAC
CCAATCTTGCTCACGAATTTCATCTTGTATGTCAGTTTCTATGTTCATGCAATTGGATCTACCTCTGAGGAGAGTCCTCACCTATCAAGTAGTG
TGTGTCGGGGCCAGTCCCAGCAAAGGCTGTGGAGCTTCAGGGATGCTGTCCATGTTTGTCTCCPAGCTGTGAAGAGCTTGGTTGACTTCACCGA
GCCTATTCCCCTCTAGACTGAGCCAGTTTGCAGTCAGTCACAGGAAGGACTTCCCCACCTAGAAGGAGCTGTGGCCGTTCAAGGGTTCATCCCA
CCCTTGGTCTGTAGTGCTTGCCTGCCTGTATTCTAGATTTGGCATGCTGTCACACGTTTGCTCTCCTTCCAGGTGTTATTGGAGCCTGTGCTCA
AGTGGAGAAATGTCCATGGCCATAACCCTGTGGTGAGTAGTTTGTCTCTTGCAAAATGCCAGCTTCCCTGTGAGTGCCTAAAAAGTGCTGGTTC
TGAGGCCTGTGCCCAGCACAGCCAGTCTGTCCCCATTGTTCTAGCTCATTCAGAGGAGGGGAATGGCGAGCCAGGGATTTCTCACGCCTGAGAA
TGGGTTTGCACGAAGCCCAGGAACCTCCCGTGAGGTGTCTCCAAGGTTCCAAGTGGCTTAGCTTTCCTTGGCACTTGTTCCAGGTCACCAGGTT
TCTCGATGAGCCTCTGACTTGGGTTTGGGCTTTACATTACCCAGGAATGGATCCCTGTGGTTTACCCTGGAGCTACCCATGTTCCTTGCCAAGG
TGGGGCTAGAGAGCCAGGGACTGTAATGTGCATGAGCCTTCTCCCTCCTACACTCTGAAAGCTTTGTGCTGCCTGCTTGAACAAAGACTGATAG
AAGCAGATGCTACCAGAGGGGATGAAGAAGGACTGTGGTCCTGGGCTCCCGTGCTATGGTATAAGGTGCTTAGTATTTCTCAGCTAAGAAAATC
TGGTCATGTAGGTTTAGCAGTGCCTCGCAGTACCTCGGCAATACCACGTCTAGAGTCCATTAGACTCAGGCCGACAGCAGAGACAACTGTGTCT
GCTGGAACCCGGCTTTAGGACCCACTTGCTCACTCACTCAGGATTTATTACATACCTTTTATGTGGCCAACACTCCTTCGCACTCTAGGACACT
GAGGAATTTAATAAAGCCTCTGGTCTCCTGGCCCTAACATCCACTGTGGAGATGAAGGAGACAGGTATTAGTATGTGGTGGCGAGTGACAGGAG
CTGTGAGGAGAGCTACAGCAGGTGAAAGGATGCTAGTTAGATAAGACAGAAGCTCACAGTTTTAAAATAGGAATCGCACAGAGCTGTGACAGGA
ATCTCACTTGTGTGCATGTGGGAAGAAGATGGGATTGTCAGCCATCTTTTAGTATAACAGAGATAATCCATTGCCCTTCATANNNNNNNNNNNN
NNNNNNNNNNNNNNNNNNNNNNNNNNNNNNNNNNNNNNNNNNNNNNNNNNNNNNNNNNNNNNNNNNNNNNNNTAAGACAAGGGTAGTTTTAGAT
GTCTGAGACTAAAGGAGACACTTCACAGACCAGAAGTACAGTCTCAGAAGGTCGCATAACAAGTGTTATCCTTACCATCTCTTCATCATCATCG
TCATCACCACCATCATCAGATATATTTCCACTCCTTTCTGCTGGTTGGAAACACAAGACTGCTGCTTGTGTGACAGTTACTGGCTCACCACGCT
GGCTCCGTGATGCAGAAAGTCGCCTGTTTCCGCAGTCATC0AGCTTCTGACTTTGCTGAGATTTTCTTTGACCATAACTAGGAAAAGCCTTTAG
TTCTCAGTGCTGAGCACCGAGTGCGGAAGTGCAGAAGGAAGCTGGCGTCCTGTCACATGCCTGTGATCTCTCAACCCGTGAAAAGCTGAGGCAG
GAAAACCCCAAGTTCCTGACCATCCTGGTTGTTTATAGACTCGGTCTTCCCTCTTCTCTCAGACAGACCCCATCCTTATCCGTCCTAAAAACAC
ACACCAGCACACACACACACACACACTCGTGCATATGCACACGTAGACCCTAGAGAGAGATGCTATAAGATTACTAGATTACTAGAGAGTAGAC
TGCAGCAGGAAGTGCTTTTATCTGTAGGTGACAGAAGATTGAGGGCTGGCCTCTATTTTATCAGGGTAGGTAGGACATGCATCTCTATGATAGG
CGACATTTGTCAGAGACCTACATGTGAGGGAGGGGGCACTGCAGGCACCTGGAGGAAGAGTAAAAGAGGCAGGTGCCAGAGCCCTGAGGTGGGG
GCCTGCTTGGCATGCTTGCAGAGCACTAAGGAAGTTTGTGTGCCTGGAAGCTGATTGGCCAGAGAAAGAAGGGTAAGAGACAAGTGGATGAAGG
TGGGTTTCTAGGGCCTGTGGTTCAGATGGGGTTTAGATTTGATTTGCACCCAATGCAAAGTCCCTAGACGGGAGATGTTAGCAAGCTCATTTTT
GGCTGCTGGATAGAGAACAGACCACAGGGAAACTCAAAGGCCAACTATGAGTGTCTAAGTGCCTTTTCCTCAGGCAGCACATCCTGGTCTGGCT
GTTAGGGGGCTCTCTTATAGTTGTGTTCACATTTGTCTCTGTCCACCTACTCTGAGATTCCTACAGCTGTCCTGAGTAAATCTCTGGCCTCAGC
CCTGCCCCTGTGGGTTTCCTGAATGGCTTTCACGGACTTCAAGACCTGGGCCCTGCCTGTACTTGCAAACTTGGACACAAAGTGCTTTGCTTTC
CTCCCTGCAGAGCCTCATGTACCATGATGCCAGCCGATCGGGCCTCACACTGCACACGTACGTGCAGCTCACCATGCTGGACCAGCATACGCGC
CCTCAGGTATGTCACAGGTCTCACTTTCAACCTTATAAACATCCTTCAGATTCAGGTGTTGTCAGATAGGGACATCATAGTATGTCCCTTTGAA
CACCTTTATGCTGTCCAGTCTTCTGACTTCAGTAAGGTCTCCTGTCCTCACAGCTGGCTGTGCTGGTGCCATCCTGAGCTCACATCTGTAATCT
GATCAAAGTTGCTAAGGATAACACACACAATGCACCTTCTGAGACTGTACTTATGGTCTGTGAGTGTCTCCTTGAGTCTCAGGCATCTAAAAAA
CTACTACCCTTGTCTTACTTGATATCAACCCTTGAGTTCTCTCTGACTTCTAGGTATGAGATGCCGGTGGTTTTTCTCTATTATACCAAAACGC
ATGCACACAAGTGAGATTCCTGTCATAGAGCTGAGTGATTCCAGCTTTTGACCATCCTTTAACACAGAATCCCTGGCTTGGGGCATCATTGTCA
CCTGGGGGCTTGTTAGAATTGTACATTTCTCAGCTCCTCCCCAGATCTACTGAGTAGGAAAATCTGGGGCTAAGGTCTAGGAGCCTCCTTTATC
CAGCTGTGCAGGACTCCTGGATATGTGTTCTGCGTGTCTACCTCTTTGAGACCAGACCTTTGTGAGGTCTACTTTCAGATTCTCGAGAAAAATC
TGATTCACCTTGGGTCACAACCAGGTGTTGGGGCAGCACAGGACACACAGCGATACTTAGGTCCTTCCATTAGCAGGAGCAGGTTCTCTTAGGA
GGTCAGGTGGCAAGGGTGACCATGGTTGCTACCTATGGGAAAAGCTTTATAGACAGGTAGACCCTTTCCCTGTAGGAGACAGGATACCCCTATA
GCCACTTACGTTAGCACCATCATTAGGCTGAGCAGTTGGCTCCAAGTGTGCCTCCCTCCAGCTGTACGTGTGATTCTTCTGTCGACATCAGTCT
GTATCTTTACAAATTGACAGATGGGAATAGATCTTTAGAGATATGAGATTTTGTCCTTTCCAAATGGTTTCCTAAGTGACAGAGAGCAGAACAC
CTGCACTTAGCACCTTCCCCTTGGTTTGCTCTGCTTTGCCTAGTGGGCATTTTAAAATTATATCCGATGATCTGTGAATATTATGGGATGTGGG
CTGAAATCTGCTAAGGAGGCAAACGCAGAGCTGGCTGGACTCAGCAGGAGAGAGATGGCTGAAGAGGGAGCCGGAAACAGATGGGAAAGAAAGA
TAGGAGCAGCACGTGCAGAAGGGACAGAAGGAAAACAGATGGGTGCGCAGAGCCAGGAGGCCGGGAGGTAGCAGTCGAGTGGGCTGTAGGCTTG
AACGTGCTCTGGGTCACACTGTCTACTAGGTGTCGGATGGGCAGGTCCCTCCTGCTCTCCAAAAGGACGTAGTAGACGAAGGGAAAGATGATCC
ATGGAGGGTGTGAGCAACTTGCAGAAAGGTGATGTGCAGCCTCTTCTCTCTTGGTAAGAGTACTTTTCAGTTCCAGCAGGCAGTGCCTCCCTAA
CCCTAGTCTTTTGGGGCTCTGCTTCAGACACCCAGGTCCCTGGCTCAGGGCTCCACTCACCAGACTGTGTCAGCGGGGCAGCTTTGCCAACGTG
TTCCATATCCTAGTGAGTCTCCCTTCTCCTGTGAGAGCAACTGTGCGGCTGCTGGCAGCCCTGGGCCCCCACATGCCTGCCTTTACTGTCCTCT
GTCCTCTAAACTTCAGGATGGCTCAGGTCAGGGTGGAGGGAGACACACAGTGACTCTCATCGCAGATTTCGCTTCTCTTGACTGTTGTCCTCCC
TCACAGTCTCTGTCCAGCACAGATGGAAGGGACCCCTAAGAGGACTGGGGCAGTTCTTCAGGGCTTCCTGTACCTTCACTCCTCGCCTGAGAGT
AAATCCCTTTTTCCATTCCTTTAGATGTCACCTGTACGGTTGATGGAAAGGTCAATTTACAGCGCAAGATACATTTTTGTAGAAAACCTGTATA
GAAGGTACTGTAGCTTGTATAGCCCATCATTTATTTACTGATTTTGTGTATGTGTGTTTGTGTATATATATTAATATGTATATGTGTGTTTACG
TGTAGGTGCGTGTGAGCATGTAGGTGTGAGTGCATAGGTGTGGGTATGTAGGTGTGGGTGTGTAGGTGTAGGTATGTAGGTGTGGGTGTGTGGG
TGTGTAGGTGTGAGTGCATAGGTGTGGGTGCGTAGATGTGCGTGTGTAGGTGTGAGTGCATAGGTGTGGATATGTAGGCATGTGGAGGCCATGG
GACAGCCTTGGGTGCTGGTTCTCAGGAAGTATCTATATTACTTTAATTATTTTTTCTTTAGGTTTGTTTAGTTTTATGGGTATGAGTGTGCTTA
CATATATGTATGTGTTCCAGGTGTGATCATGGAGGACAGAAGAAGCTATTAGATTACCTGGAACTGGAGTTACAGATGGTTGTGAGCCATGTGG
GTGCTAGGACCTGAATCCAGGTCCTTTGAGATAGTAGCAAATCCCTGAGCCATCTCTCCAGCCCTCCACTGTGCTTTTTGAGACAGGGTTGCTC
ACTTCTTAGTTATCGCTTCTAGGCTAGGCTGACTGGCCAGCATACCTCAGGGATCTGCCTGTTTCTAATTTCCAGTGCTAAAATTGCAAGCAGA
TAGCCTCATGCCCGTGCTTTTACATGGGATCTCGCAGCTGACTCGGCTCCCCATGGTAGAACAAATACTTTACCATGAAGAGCTGTCTCCCTGG
CCCTAAACTATTGCTGGTGGAGTTAGTTGCCTAGTTACCTGACAGATTCGGAGTACCTTCAGTATTGATGAGCTTCGGTTGCTTTATAATGTTG
TGTAACACCACCAACCCACTAGTGACACCCACCTTCCTTTATTGCCAGGTCAGTTTAAGGAGGTCAGCCGAGTGGCTCCTTTTACCTTGGCTGG
GCTTAGCAAAATGCCTGGCAGTGGGCTGGACTAGAATGGACTTGACCCAGGTGGTCCAGGCAACTCATTTCTCTTCCTCTCACCTGTCATCCCC
TCTGGGACCAGAAGCAAGCATAGCCCCTGTCATCTCATGTGTGTGGCATAGGGCAAAAGTCCCAGTGGACACACAGCGGCATGCTTACATTTGT
TGTTCCGCCCTTGCCCACATCCCATTGGTCCCATGCCTAGGGATGGCAGGCGAGAGGGCCAAGGGGGTCCATGGCACAATGCAAGCTTCTGGGA
AGATGGAGATTTGGGATGCTTAGAGCAGTTAGTCTGCCCCGTGCTTATGCTGTGTATAGCTTCTGTCCTCACTTATCTAGACAGTTGCAAGGCT
TCAGGTGCCCACCCGGGCTTAGGGCTCCAGTGTGGCAGGGCCTAATGTCTGCTCAGGTTATTGATAATTTGGGAGGTGTCTTAGAGGAAACAGG
TTGCAGACTGAGGTCTGAAGTTTCAGCCTTTTCTGAGCTGTTTCCATCTTCATACCCATGTATCTTGTCCTCATGTCCTTGTCAGTGCCCGATT
CTTACCACCTCTTCCTGCTCTGAGCCTGCAGTGCCACTGCCACTTCTTAGGCAGTCCTGACACTTGTACAGAATGTTCCTAGCTTCCCAGACCC
CTCCCTCCCATCACAGCTGTCCTTATGGTTCTGTCTCAGCCCTTCATGTCTCTGTGTGTCTGTGTGTCTGTGTGTCTGTGTGTCTGTGCCTGGT
GCCCATGGAGGTCAGAATAAGGCATTGGATCCTCTCAGACTAATCTATGTACACAGCTAAACTCCATTTCCTAGGTGGAAGCAGCAAACAGTTC
AGACAAAGGATAATTATCTACATCTTCCTAGTGGGGCAAGCAAGGTGTGCGGAATAGTGAGAGGCTCCGATTTCTCTTATTAAAGGTCATCCCT
CAGGCACCACTGAGCCCCTCCTCTCTCCACACTGGCTTTACTCATGCCCTCTATTACATACTGAGCTAAAGTCCCCTTGTACTGAGGCCGCCCA
GGGCTCTCTGTGTTTATCAGGCACCTTCTGGTTGAGGACTGTGATGTAAACGACGTCCACCAGAGGGCAGCCACTGCGTAACTACAGGGGTTCC
TGCATCCCTATCCTTTAGCCCTTTTCCTGCCGGGGAGGCCTGGCCTTCTGTCCTGGGATACAGACTGAGCTCTGAGAGTTGTCCTTGGGATTGA
GGGTTTATGGTGCACGGTTTCCTCTGTGTTGTACTCAGCCTGTGGGTTCCATTTCACACGTGGGACCTGAGCTTTTGCTGGTTGGGTTTTTACA
CTTCTATGGTAAAGAGTCCCAGGTTTTTGTCCAGGTCTAGAGTGGAGCAGCTGTCAGCCAAAGGCTTTCTGTTTTGACAGTGAAAAGGGTTTTG
ATCAGAGCTTCAGAGGACCAATAGGACGAGAAGGGAGTTTGGGGTTCTTATACGGTCACTCTCCTCGTAACTCCAGCATGCCCAGTGCACCGGT
GTCTCACAGGGGGTGCTATAGAGAAAGGAATTGTCTACAGCTTTCTACCTCCTGTTCCCATCTAATCGAGAAATTAGTCACCTTAGGTAGAAGG
TGCTGAAAAACAAAGAGGCTGATAGATCCAGGACTGTGCCTGCAGTTTATTAGGGACTGGCGGAAGAAGCGGCNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NTAGCTATCTGAATATCTATTTGCTCCTTAGGGCTTGAAATACACGCCAGGTTCTCATGGTGGATAAAATAGAGTTACGACAAACATGTATACA
AGCCGTCAACTTGTTTCCAGCCTGAAAGCTGGTTCTGTAATGCAGAATTACCTTTAGTGGTATCCTTAGCCTGTATTTGGACTAAAATGTCTTG
GTTCTTACCTGTGGAAGCTCAGCTGGGCACTGGTATGCTTCCCCACTTCCTGTGCTGCCCATGGCTTGCTAAATGTGGAAGTGGTGGAGCCCTT
TCATAGACGGGTCTAGTCATGGCTGACCTGGCAGTGCCAGGTTCATCTTTTCCCCAGGGATCCCTAGACCCTTTGCTGAGGATCTGGACCTGGG
GGCCTGGCTCTGCAATCTGTAATTGAAAGGTTGGGTTTCTGGAGAAGCCCACAGATATGAATTGGTGTTCTACATTTCCTCAAAGCAAAATTTC
TCCGTGTTCTTGCCTTTGGGATCTTGAACAAGTAACTGCGAAGGAGAGACATACCTCTTCTGGTGAGGTAGGAAGGCAGAGTGAGCTCAGTTAC
ATCACTAGCAGTCCGAGAGCTTTGTGTGGATTACTGAAGAGGAAGGTCAGCTTTCATGCTGTCCTCAGAAACAGGAACTGAGCTGTGTGCACTG
GGCTGCAATCCAGAGCTAAAGGTGGCACAGAAGCGTTATGGCCAAAGAAGCCACGCAGGCAGTTGTGTAGTAAGCTCTGTGGCTGAGGGATTGA
GCAAATGTTTTACAAGACTGCTTGGTCTTCCTTTTCCTAAGGTCACGGTAGAGGAATGGAGTGGGTGCAGCTGGAACATGGCGTTGACTCACCT
GGCACATGGGAACAATGGGCAGGTCCTCCCTACCTCCCTGCCCAGCCCCAGAACCAGGAGGCCAGGCCACAGAAAACACGGGGATCCAGGTTAG
CCCCTCAGGGAGGACAACAATGCAGTGGAGAGCAGGTGGGACTGAGGGAAGGCTCTGCTGCAAAACACAGGGCTAGAAGAGGAAACAGTGTATT
TAAGTATCAGGTTTCTGTCTTAATTTGTTTGAAATAGTGTGTTATTGTGTAGCCTAGGCTAATCTGGAACTAAGAATTTTTATGTAGCCCAGGT
TGGTGTCAATCATGAGATCATCCTGCCTCAGCCTCCTAGTGCTAGGATTACAGCAGGTGCCACTTGAAAACCAGCCTTTGATTTGGTTTTTTGA
GCATCAGGTACTTTCAACTGAACTAAATAGAAACATGGTACAAAGTATGGTGAACAGTGGGACTGTCCATGGCTCAGCTAAGGAGTTGAATTGG
ACTCTCTGTGTTTTGGTCAGGGTTACTATTGCTTAATGAACACCATGACCCATAGCAGCTTGGGGAAAGAAAGTATTTCACTACAGTTCCATGG
ATCAGCTCAGCATCAAAAGCAGTTAGAACAGGCAGACTTAGCAGGACAGGACCTAAAAGGAGGCAGGAGCTGATCCAGAGGCCGTGGAGGGGTG
CTGCTTACTGGCTTGCTCCCAGTGGCTTGCTCAGCCTGCTCGCTTATAGAACCCAGCGTGTATGTGTGCTCATTATACACAAGTGTGGAGCCCA
GATAAGGACACAGTCCCTGCTCTATCACATTTATACCTTCTTCTCTGAGACAGAGTCCTTGACTGAACCAAAGCTTGCTTTCGGTTAGACTTGC
TGGTCTAGGAGCTCTATCTGTCCCAGCACCACAGGGCTGGGTTTAGAGGCTCAGGGCCATGCCTGGCTTTTTTTTTTCCCTATGGGGACTTGAA
CTCAGGCACTAATGCTTGCAGAGTAGGCACTCTTATTTACTGAGCCTTCTTCCCAGCATTGCTTTTTTAGTCTTAATATTTAAACTGTGTAATA
GTGCCGCCTCACTCTTGTTCTGAGCACTGATACTTTGCTCAAGTTTAAAAAGTAAAGAGCCAAGGAGGAAGAGTCTACAGTCTGCAGCTCAGAG
AGAAGCCCTGCAAGCATTTCTGTGTATCTTTTCAAATGAGGACATATGTTTTTTTGTGTGGTTGAGGCATATGCATACAATTAGCCTCCTGTAT
CCATTGAGCAGCATCAAGTTCAGAGTCTTCCCCCTGTCTGCGTATGACTTTCCTACATCATCCTTCAGCTACTGTGTGCTTTTGCTGCAGAGAT
AGATCAAGACTTCATTTCTCACAATGCTGAACAGTGGTGCATGGCGAGCATCCATGAGCATTTTCACAGAGAATGAGTTCTAGAAGTGAAAATG
ATTGGCTGATCCACTTCCCAACTTGAATGATGTCTCCACGTGTAGAGGGCAGATAAGATGCTTTGTCTCCTTGGAGTTCATTCCAGAGTAGGCT
CCATCAGTCACTGCTGGGGCCAGCTCCCATCTCCCAGTGGTGGAAGGTCAGACAGACAGATGCTCAGGGTCCTGGCAGTACCATGTGGGCATCA
TCTGTGGACCACACTATGCAACATGCTCTTGGTGCTGCCCTTCCAAACAAAAGCCACCCCTTAGTCCTTTGGGGGAATGACACAGCACATGGGT
GAATAAAAATCTTTGACCTCTGATTGCAGTGGGAAGATGCCCGAGGTTGACTACGCGATTCTGTCTGAGTGGTTTGACTGGATCGTCAGGAACA
TTGATGTCTCTGTTGATCTGATAGGTAAGACAGTCTCTCTCTGTCCCCTACCTGGAGGCTTCCCTGGGCACAGCTGGGGTATACTTGGGAGGTT
CTACCTTGGATCGACAGAAGGCAGGGACACCTATCACCTGTGATGCCTCCTCTATCACAGCTCACCAAGGCAGGCAGCATTTAGGGGGTTCTTC
TGTCCATGAAGGAACTCTGGCCTTGGTTCCTTAAACTTCCTTCTTCTGGGTCAACTACTTCTGGTGGCCCCCATTTGTTCTGTTTTGATGGCTT
TTCAGTATTGTGTGGGAGGGACAGAAATTGAGTAGTGGCTAAAGCCAATGGCTTGCCAGACCCTGTCTAAGAATGGAATTCCTTTAAGCTTCAT
GATGATACTGTAGGGCTGAGGACAAGAAAGGGGACTGGGGCCTGAGGGTGGGGAATCCTGTTGGTCCCACAGACTGAGCAGGCTTGGTGAGGCT
CCTGCACGCTCCACCCCAGGCCTGTGTTGCCCTTGTCTGCTCCCACACCAGGAGCACAGCACTCAGTCGCTTGTGGTTTTACTTCTCAGTTTAT
CTGCGAACCACTCCCGAAATCTGCTACCAGAGATTAAAGATGAGGTGCCGGGAAGAGGAGAAAGTCATTCCGATGGTAAGAGACCAGAGCCACC
TCCACCTCATCCTGCCAGCCCTCTCCTTTCCTCCACCATGACACTATCACGCTTATCTGGAGCAAAACCCAGGAAGTACGACTGAGGCAGCTCA
GGCTTCCTCTATGGCTTTGGAGCCCTTGTGGGTCCCAGGGCCACTTTACTTCCTATGTTTCCTGACTCAGGCCTCTGCACCTCCATGTACACAC
ACACACACACACACACACACACACACACACACACACACACACACACACAGTACAGGCACCCCCCCCCCCCCACACACCCACTGTGTATATACTC
ATTTGTTCTGTACAGTAGCTTGTCTTCACACACGCACATGCATGCACTCACACCCCATGGACTTAGACAAATGTGCCTTTGCATAGTAGCTTCT
CTTCAGCTGTGATGTCACTTTCCTGGCCGTTCCTTACATTGCCCCTAGGCCTCCTATGGTGATAGCCCTCAGAACAGCTATTGGCAGAGTTTGT
TCCCATTTATCTTTACACCATAAACACTTTTAGATTTTGGTTTTATTTCTTCTGTCTTCAACATGTGTCCTGGGGCCTAGCACCTAGCAGTTGC
TTAGTTAATACTGGACTGAGTGTGAGTACTTTATGTGCACAGTTGTCCTTGAAGGATAGATAGGGGCAGGCTCTTTTGGGACATCATGGTGCTC
TGAACCTAGGATGAGACCTAAAAGCCAGGTGTGGTGGCACACACCTTTAATCCCAGTACTCTGGAGGCAGAGGCAGGCAGATTTCTGAGTTCGA
GGCCAGCCTGGTCTACAAAGTGAGTTCCAGGGCTATACAGAGAACCCTATCTCGAAAAACCAAAAAAAAAAAAAAAAAAAAAAAAAAGATGAGA
CCTGAAAGACCAGTCTGATCCCTTAAGCTCAGCCAGGTTTTGTTTTGGACTAACAAGAGACATCCCAGCCATGAAGGCCTAGGCCTTGCTCTGG
GCACTTCGTTTGAAATCTCAAAGACAGGAGTGATAGAGGATGGAGCATTGTGGGCTGTGCTGTAATGAGGCTTGGCATTTAGAAATGCCATGTC
TGGAGTTCTTCCAATCCATTATCCAAGGGGTTGCCAAGGTCTTTAGGACAAGCTTGGCTCTAGTACCAGGGGGAGTCCAGGCTGTTCATGTCAC
TTCCTAATAGTCCTTGAGAATTCTGCCCCGCCCTGTCCCTGTGAGACCCTTGGTGGGTGGGATTCCCCCCTCCCCTCTCCCTACAGCCCTTGTG
TTTCCATGGCAGGAATACCTCCATGCTATTCACCGCCTCTACGAGGAGTGGCTGGTCAACGGGAGCCTCTTCCCAGCTGCAGCCCCTGTTCTGG
TAAGTCACTGTCCCTCCCAGAGAATGTCACCTGGACAGAGACTCAGAGGGACTCGGACTTGAGGAGGCTAGTTATTTTAGTAACATGTCGTACT
CATCATCACCACAAAGAAGTCCTCAGTTACTCTGGACCTGGGTTTGCATTTGGACCTTTGGGGATGATGGTGTCCATTCTTCAGTGGGTGACAC
GGAGCCCATCGTCACCCATCGTCATAGTTAGGTGACTGGGCATCTTCTGAAGGCATCTCAGTGGTTGTCCTCAGTTGTCACACAGTGGCACCTC
AGGAGCTGCTGAGGTGTAAAAGTGCCACGCTCCTGCTGCCTCCAAGCTGCAGGCCAATGTAGCAGTGCTGGTGTGTCCTACCTATCCATATGTC
CTACTGTGTGTCCTGTGCCCCAGTCTGCTCCTCCTGAATTTCCTCAGGCAGGCAGGTGTACTTGAGGCCGTCTCACATCCAGCCGGTGATCATG
GGCTTTTGTTTATGCCCTTGGGCTGTAGGCCACAGAGACATCCTGGTGCTAAGAGACAGGTGATAAGGGACATGTCACTGTGGGAACTTTCAGA
GTTTATTGTTTCCTTCACTGTGAAAAGCAAACCTGAGAGGGCTGGGAAAGTGGCAGAGTGGCAGAGAACTTGTCTAGACATTGGAGTCCCCGGG
TTTAGCCCCCAGCAGAGCCCCAACTCCTGTCTCAGTGAAAAGAAAAGGCATTGTTGGTGGGATGTGGAAATCTGGTTGAGAGTGTCAGTTGCCC
TGGAGCCAGACTGTGTGGTTCTGACCCTCTTGGCCTCTCAGCCTCTTGCCTCCCCAGTGGGGATGGAGATGGCCAGTGCAGCCTGTGACCTGTT
TGTTTTATTTCCAGGTGATTGAGGCTGACCACAACTTGGAGAAAATGTTAGAACTCTTTGAACAAAACCGGGCCCGGATATTAACTCCAGAGAA
CTGGAAGCATGGACCCTAGGACTGAGTGATCTACAAGACAGCGCCAGGAACAGCCCAGCCAGGGCCAGGTTAGCTGCTAGGAGCATTTGGAAGA
ATCTACTCTTAGGAGGGTCTGTTATCTAATCAGATCTATTTTCTCAATGGTCTTTTCACTGCAGTACAAATGGCTTTTGTCTGTCAGATGCTTG
CAGCATACATCCCTCCTGGTGCCCCTCCTGGTGGCTTCCTAACGATGAAGCCCTGTGTTAAAATCACTGACTGTATCCCTGGGGTTGTTCTGGA
TCCTCTGCATCAGGTCACCTCATGGGTGAGGGAGCAGTCTTCCACCCCAGGCAAGGATTTGAAGCCCAGAGAGGTGATGGGACTCGTCAAGCCA
GAGCATTGTGGAGGGAGCTGGGACCATTCAGATACCTGGTTGGGCTGCCTCCTCCTAAGCTGTTCCCCACCGCCCTGCTGCTCTGAAGCACCAG
CCCCCGCCTCTGTGAGAAGAGTGGACAGTTTGTGAATTCCATCATCTTTCAGTACCGGGCCAGTTCAAGGAGACCAGACATCACTAGGTGTTGG
CACACCTCTCTTATTTGGCATGAGCCCAGGGTGTCCTGGTATGAGGGTACTGTGACCTCTATCCATTCCTTTGTCACCTGGACCTCGGTGTCCA
TGGGGACTCCTGGATCGGCCTCTGCTTGGTGCCAGAGTCCTTGAGGATGAGTCCTCCCCAGACTCTACCATGTTCCCTGTCGTTTTGTGTCAGA
AAAAAGGGGGTTATGGGACCTGGACACTTTACCCTTTGCTGAGCGAGGCCAAGGGCAGCCAGCTGAGCTTCCAAGACCCCATCACTCTCTCCTG
TCCTTGTGTGAGGAGCAGTGTCCCCTTGCTCCAGGGAGAGTGACAGTGGCCTTAACTCCTTTGAAGTGTTTTTCCATCTGAGCAGATGTCATGG
ACCACGAGCATGAAGAACTCATCTCTATTGGATCAGAAAATACCTTAGCTTTGCTGAGCCGTAACTGTGCACTGAAGGTGCAGCTGTTACTTGA
ATCAGTCATGACCAAGAGAGGCCGGCCATTCCTCAGGACCGTGAGACTGGAAAGTCATGGTTTGCCAACAAAGACAATTTAAGCCTTGCTGCTC
TTGACATTCTAGAATGGATGGAGTGCATCTGGAAAAGACTGGACACAGATGATACAGGGGAATGATGACCGTATACCCTGGAGAAGCACAGCAC
AGGCTGGGGAGGGGGCTGCAGGGGAGCCCTGCTCCCCTGTTTAGCTGCCTGGGAGCTAGAAAGAGGGCCAAGAGTCCAGCTCTTGGTCCTTTCT
GTTTGGACCAATTCTTGGCCCTTTTGGTAGTATGAAGAGTAGCATCCAGGGAGATTGTCCATCAGCACCTGCAGTCCAGCAATGGATAAACCTG
AGAAAGGCAGGAACTTTGAACCAGCAGTACTCATGGGGGCATCTTTAGAATTTGCAAACGAACAGTCTAGTACTTGCCCTTTGCTCTACCAGAA
CACTTGTTTTGGGGAGAAATGTGCTGTTTCTCTTTCTCTCTAGTAGCCAATAAAATTCTACAGCTAAACCATGATGTAAATTCATTTTCATTCA
TTCATTCATTCATTCATTCATTCATTCATCCATATTCTCTCTCTCTCCCTCCCTCCTTCTCTCCCCCTCTCTCTTTCTCTCCCTCTTCCCTCCC
TCTCCTTTTCACTCTCCCTCTCTTCTTTGAGTCAGGTTATATTTTATAGACTGGCCTGGCCTTGAACTCTCAGAAGTATTTTTGCCTCAAACTT
TTGAGTTACAGGCATGTGACACTATGTCTGGCTTGTGTGTTCACTTTTGAGTTGACTTTATTTTATTTTTTATTGTTATGTGTTTGTGTGTTTT
GCCTGTATATAAAAGTATGCCTGGTGACCATAGAGGCCAGAAGAGGGCATTGGATCTTTTGGGACTGGAGTTGCAGTTGTGGCTTGCCATGGGC
ATGCTAGGACTTGAACCTAGGGTCCCTAGAAGAACACCCAGCGTGCTTAACTCCTGAGCCATCTCTAGCCCCATGTGTTCATTTTTCAAACACC
ACATTTTATAGTATAGTGCGCATGGCTCAAAAAAGAGCCTGCTGTCCTTGCACTTCAACACTGCAGACAGGAGATCATCTCCAGTAATGGCTAA
GGTTTGCAGAACCTTCCAGCTCAAAAGATCACACTCAAAACAGCCAGAAGCATCTTTCCCAAGGACTGCTATCAGGCAGACCTGGTAGGGGCAG
GGGTCATGGAGCAAGTACCAGCACTAACATCTCATGGCCAGAAAAGGGCAGGTAACACATGGTGGGCCACAAGGCCTAGTTCTTAGACACAGGG
CATCACTGCCTTCAGCTGTCTCCGAGTGGCTGGTGGAGCGTGGCAGGGACTGGCATCCCTGAAGAGCTAGAGTGTTGTCACTTGATCCTAACCA
AGACCACTTATTCCTTTCATCTGGGTCAGATTTCACAAACTGCATTTTGGAGGGTCCCTTCTCAGTGTTTGGAAAACGACCTGTGGCTAAATAA
ATGTGGTAAACACTCTGCCAAGCAAGGTGCCCTTGCTTCTGGCCTCCACGATTCCTCAGGCTAAGTAGTAGGCTACTGAGGTGTGGTATTCCTG
GAGGGAGAGAGAGGTACTTCCCGACCTCGTGGACTCGCTTCCTAAACGAAAACATTCTTTTTATTTTGAATCATGTATATGCCCATGTATCTGT
GGCGCCCCATATATGTACATATATACACACATATGTACCTGTGCACAGTGTGCACGGAGTCCAGGAGAGGGCCCTGAATGCCCTGGAGCTGTAG
TTTCATGTTGTGAGATGCTGGACGACGGTTCCGGCGATCAGATATGGGCTCACTGTGTATTTTCCATAGCTCCTTTGCCAGGTAATGTGAGGCC
GGCAATCTAGGGATAGTTCAGTAACTTGGGGCTCTTCTGGTAGGCTAGGTACATCTCCCTCTTCTAATAATAACGGGCAACCTAGCTCCTTTAT
ATGTACATACTGTGCACACACCCCTCCGTAACCAGGTTACCTAGCAATATGGGCTCACTACCTCTGTCAGGTTGCGCACACACACCTTGAAACA
ATGGGGTGAGTTTCTCTTCCTCTAACAACAATCAGATGGTTTCTATTCAAATATATTCCTAGTGTGTCTGTGTATAAAGAATAGAAAGGCTGGT
GTGGCCTCAGCTGGTTTTGGGTGTACATGCAGGATAGGCAAAAACACTTGTAGGCAGAATCTTCTATTTTTACCATCTTATATGCCTATGCCTA
GCAGGGTGTGTGTATGATGAATGTCAGATGCATTATGAGAATCGGTATGCCCAGCAGAAAGATGGTTAGATGTCCAAAGCTATCAATCAAAACA
GAACCACCTCTTAGTAACAAATGATTCTCTTGGGAACCGTAATCGGAGCCTTGGGCATCCTCACCCACTCGTCTTGACAGTGTGTCCCTCTCCA
GTCTGTTCCATCACTTTGCTCTGATTAGAGGTACTTGCTATGGCAAGGGAGCACATTCAGAGACCTTTTAGATCTGTAGGGTCAGGCTGGAATG
CTGACAAGGCGCACACTTTCAACCCCTCTGCCTGTAATACACACATAAAATGCCTTTAGTACACACCTTCAATCCCAAACAATGCAGCTAAGGT
TAGGTTGTAGAAGGAAGCAGCCATATTTGAAAGTGATGTCTAATTGAGTGGTAGAGTGACAAATGAGAGAAAGATTTGACAGAATAGGATATGC
CCAACTCTCATGAGGAGAGGGAAGGAGAAGGGGGAGGCGGGGGACAGGTCTACCAGGACAATATTACTGAGACAGATTGCGGGTGAAGACAGAA
CGAGCCGGAGAACGAGAAGGAACCAGAAGATTAGAAACAGACTGCCAAAGTTCCGAGGTCAAGCCGAGCAATTCAGTCAGAAGCTGAGAGAAGC
CAGTTTGAATCAGTCAGCTTGGAGAGAAGTTTTGAGCAACTGAGTTGAACCAGCCAGCCGGAGCTCAGAAAGAACTAGAAAGAGTGAGCTTATT
CAGCAGTAAGCCTCGAAGATGCCAATTATATCTGGTAAGTTACATTTACACAAATTGGTGTGAGCTCGTCTTCTTTTTTCTTTTTTTTTTTTTT
AGCCATTAAGTTAATTTATTGAACAGTTGACTTAGGCATCTACAGTGGTGACTTCAGCCTCCACTCCTGGCTCCATATTAATGGTAAGCAGTCC
GCTCGACAATCTCAGAAGGACAGTGTAGATCACTGAGTAGCTTGTGGATCCTCCTTTGGAAATGATCACGTTTGGAACCTTCACCACAAGGGGT
TTNNNNNNNNNNNNNNNNNNNNNNNNNNNNNNNNNCATGGCTGAGGCATGGCTTCCTGACCGACTTGTTCTTCAGCAGGAGGGAACAGCGGTGA
GTGGGGACGGAGCAGACTGTACAGCGCTCCACAGCCCGCACCACCACGTCTCAGTAAGCTGAGCCCTTATTTTCAATTCTTTGGGTAAAAGGCC
AAGAACCTAGAAATACTTGCTGCAACCCTGACAGTTCTGACCACCAGTAAGAAAGGCAGCAGGTTCACAGGTTCTGAGATTACGATACAAGCAT
CTTTGCATGGGAGGGCAGTCCTGTGGTCCACCATCCCAAGTCTAGCTCATACTCAAAGTAATACCTGTCCAATATTGTGAAACTTGGTCGTTGA
GAACAGGGCTGGGGGGCTTCTAGATGGGGAGATGCAGGACTTTAGGTCCTTGGCGATTCGGGCTGGTTTTGTGTGTATACTTGACAGAAGCTAG
AGTCATCAGAGAGGAAGGAGCCTCAGATAAGGAAATGCCTCCATGAGATCCAACTGTAAGGCAGTTTCTCAATTAGTGATCAATGAAGGAGGGC
CCATTGTGGGTGGTGCCATCCTTGGCCTGGTAATCCTGGGCTCTATGAGAAAGCAAGCTGAGCAAGCCAGGGGAAACAAGCCAGTAAGCAGCAC
ACCTCCCATGGCCTCTGCATCAGCTCCTGCCTCCAGCATCCTGCCCTGTGTCCTCACTTCCTTGAGCTGTGAAGAGCGAGGTGGAAGAGCAAGC
GAAATAAACCCTCTCCTGTCTACGTTGCTTTCTGGCCATGGTGCTTCAGCACAGCAATGGAAACCCTGACTAAGACACTTGACCACGAAGTGTG
TCCGTGGGAGCCAGAGTCTCCACTCTCCCTGATTTTGGAGGCAGAGGCTCATGCCACTCAGGCTGGCTCAAACCCAGTAATGGAGCTGAGCCTG
GTTTTGCTCTGATTCTCTGGCCTCCATCTCCTGAGTGCTAACACGCACTTGGGCATCTTAAAGACACTAATCACCGTGTTTGTCTTCCTATCTC
TTTCTTTAATCCCTTGGCTGTGGGCAGATTAGTTCCTTGAAGAGAAGGCGGGAGTGGCCTCCTTTTCACTCTCCCAGACCTCGAAGATCTGGCA
GACTCAGTTTCCCCCTTCTCATGGGATTCTTCTGTTTGCTTTGGATTTCTTAGTAAGTGAACCATTTCCCCAGATCTAGACACCTTAGTCCCCA
GCACCCATTGGGCCTAAGAGCCACCCACTTCTAGTTCTCAAAGGGAAGCCATGATTGGGAAGGAGAGTGAAAACGCTGCGTATGGTTGCTGAAG
TCCCCTCTCCGGCCTCACATGTTTGAACCTGTCCTGCTTCTCCACGGCAGCTTCCTGGCCTGATGCCTGCCTCTACATTTGGCCGGCTGAAGCC
TGGTTATACTCTCTGGGCCCTCAAGAGAGTGATGCGCTCAGAGAGAACCTGCTCTCACTGATGCCCAAGCACTTAGCTGCAGGGAAGCAACGGG
GATTAGTGCGGAGGTTGGACAACGGCTATTCAAGAACTTCTTTTGTCTCAGCAGGCCGGAGGACCTTCACTTTCAATAGCCCTAAGGTCCCTTG
TATAACCTATGAACTTGGGCTCATAAAGGAGAGAGATCCAGTTAAGGATGCGGCCTGGGCACAGGGTCCCAGGCCTGAGACTCAGCACTTAACT
GTCATCAAGCTCATGTGTAACACCAGGGAAAGGGCTGTTTTCTGTCAGAATAGAACCGTATGTTGTTACATGTTCTCTCCCTTGCTCTGCCGCC
AACCACAGCCACTTAGAGGCTCCAGCAGAGTCCTGTCCAGTCCAGTCAGAGTCCTGTGAGATCTGATACTTGCTTTCTGGAAQAGAGAGCATCT
CTTCATTCTGGGTCAGGAACCAACCAAGTCAGACATCGGACATTTATCGTCATTTTACCAATGGGAGAATGGAGACAGTGTGGTGCTCAGAGTC
CAGAGGGACAGTATATCAATCCATGCCCTTGGACTTCCTGGATCTAGCCACAAATGAAGCCTGACTTATCACTTGGACAATTTAGTCACATGAC
CCAGTAAGTATTCTTTCTTGCCTCTTGCAATTGAATAAAGAAGGGGGTGATGGTTGGGGCTCAGAGCCTGTCAGAGGGTCAAACTCTCCATTTT
AATAGAAAACAGCCCTGCAGCCCCTCCCCGGGCCCCAAGGACTCAGCCTGATGAGGGTGACAGTTTGGTGGCTGAGACTATAACAGGGCTGCCG
CCTTGGAATTTCTTGGAAATGTGCTGAGCAGACACAGCTCTGCTACAGCCCAGTGGTCAACGTATACCTAGCTCCCTAAGCTCAGACCCATTAG
GCTTTGGCTAGATAAGGGCTCTTGGGCTCCAGAGAGTTAGCATGGGCTTGGGAAAGTACCGCGAAGCTAAACAGAACAGACCCAGCACAATAGA
TCGCTCTCTCATGATAAGCGGTGGCCTTTATATCCAGTCCTTGGACAACCATGTTAGTGTCAGAGGATCAAAGACGGGAGCTGGGAGCAGGTGT
GAGCTGCACCTGGCTGTGCAACGAGAGCCTTGCATGGAGAGGGTACTTGTGTGGGCACCATAGGTTGTCTGAGTATACGTGTAGGAAGGAGGAG
GGAGTTATGTAATGGTGCCGTGTGCATGCACACGGTTTGTATATATGAACACAGGCTTAACCCAGTGATGGTGGAGGGTGCGCATGGTAGCCAT
GGACTGCTGTTAGCCTCGCTAGTGACTTGTTCTTTCCGTCTGTGCTTCAGTGAGCCCTGGTGCTTAGCAGTGGCAGAGGCCTCTGGTTGCTACC
CAAAGCCTGGGACTTTCTGTGCCCATCCATAATGATTCAGCTACGGTATGGCTTTGTCTGCCACCCGCTGTGGCTTCCCTGTGTGAAGGCCTCT
TCTGATCTAAAGAGCCTGCATGGCAGGAATCAACACTCTGTAAAAGGCTGCCTCAAAACAGCGCTGCTGAGAGCCAGGTATGAGCGACACGGAG
CTGTCACCTCCGAATGCCTGACATCGAGGCCTTCAAGGCATGGTGTACCCTCTCTCTTGGCTTGCCACTGGGACAGTTTGATGGCCTGCATCCT
TTCAAGCTTTCATCTCCCGCCACCTCCACCTTCTACTTCCCCGATGCCATTTCCCTGACCGGCGCTTTTGATAGTATGGCCATGTACATGGAGT
TGGTCCTGCTTCTTCCTGGCTTACCATCCGAATGATGGACCATCACATGCCATCTCCCAGGQATTCCAGGCACCCAGCTGTTCTCCTTAGGCCT
TGTCCCTGGCAAACAGAGGGCTCAGGCTCTTTGTAGGAATGGGCAGACCCCTGAGCAGGATGCCTACAGCCGAAGGAGTACGTGGATAGAAGAG
ACAGGCAGAACCAAGAGGCTGAGAGTCCCAGGAGTTTAAATCGTTAGAATGAGCGTCAGTGTAGGTGGTCCGGGTGGGAGTCAGCATGTGGCTG
GACACCAGCACCATAAAGGAGGTAGAGCCCCACAGTGCAACATAGTAGCAAACAGAACAGAGGGCCAAGTGTTTTGCATAGAGAGGAGAATGCA
GCCAACAGGAATTATCAGAAAATGAGACCCAGAGACAAAATCTCCTCTTGAAAATCAGCGGTATAATACAAACCCGGGATCCAAACATCAGGCT
CATATAGCTGTGAGTGTCCCACAGCAGCAGTGGGCTTAAAAATAAAAGGGATTTCCTCCTCCTCCTAGGGTAGAGTCTGTGACAAATGAACAGT
GGCAGCAGAGAGCCAGCAGGGGGCAGTGTGTGCTTGAGTTAGGCCAGTTGCAGGACAGCAAACCTAGGGATTTTTCAGAAGGAACCTGTGGGTC
CCTGTCTCATAATACCCCCGACACCCCCATCTGCCTCACTTTGTTCAGGGTCACACCCCAATTTTAGGGTGTGATTTTTGGGATTCTCTGTGCT
AGCGGAAGGAAAACATGTCCGAGAAAGACAGTCTCTGTGCGTAAAAAGAGGTCACCAGGCAAAGCCTCTGTCTGAAGGGCAATGAGAAAGATGA
GTGTTTCTGCAGAGGGAAGCATCGAGCATATTTCTATTTTCCTCAGAAGGATAAGCCTATGGAGTACTGAAGGCCAAAAGATGGTACCTTGTGT
ATGTGGAGTCAAGTTCTGAAATTGGTGGCTCATCATATCCTCACATCAGACAGGTCTCAGAGGAGTGGCAGCAGGCTCCATATGAAGGGGGCTG
GGAGATGGCTCAGTAAGTGAAGTGCCTGTCGTGCAAGCACGTCCAAGTAGGAGGATCTGAGTTCAGATTCCCAGTACCACATAAAGTCCAGGTG
CACAGCACACCGGGGCACAGGACGTCTGGGGGACTTGGGTGGCCAGCCAGCCGGTCTAACTTATAAGGGATAGCAGAGACAGGAGGATCGCCCC
CTGGGCTACATAGCAACATTTGTTTCTAAAACCCAAACACAAGCAGAGAAACAAAAGAGGAAATCCCATGAGACTGTGCAGACACCCCCTCCCT
ACAAGAGCGCCACCTGCCGCAAACTTGAGAAACAGCCAGCTGGGCGTCTCAGCGTTCAGAGGGGCATACTTAAGACTGCCTGTTTAGAGGGTTC
CCTTCACCCTCTGAATTTGCAGCCACCAGGAACCCGCCGGGGCCCCTATAACCCCTCGAAAAGTATCACTTCCTTCTACGTATTTGTTGATAAT
GAGCATTTATTTTGAGTACAGAAAGATACATTGCTGCTTATTCAGCCTAACCTAGGAAGGAGAATGCGCTGTTCCGCAGATCCCTCCGACGTGG
TGAATAGGCGGGGATGTTTTTCTGCCCCCACCACTTTCCAGAGCTCTAGAAACAGACTGCCTTTCTTAAAAAGAGTCTCATCCCCGGGGTGTGT
TTTAAAGGCTGGACAGTACCACAGACAGACATCTTTGCCTAGAAAGCAGAACGCCACAAAGGTGGGCAAAAGGGCTTGTAAACAAGGCTCCCTC
CCCCAGGCCTGGGACTCTAGGGTGCTGCCCGCAGCCTGCAGCCCCTGCCGCCTAGATGCTACAGAACTGGTGGTTCGGCCCTTTCGGCTCTGCA
TCAGAATAAATGCTTTCTCTATAGAAAATAAAAATCCTTGCTACCCCTCTGCTTAGGAAGGGATTGATGATATTTTCTTAGCCCTTCATTGGAG
GAGTTGAATGTGGGAAGGGGTTCAGTGGGGATGGGTCTGCTCTTCTGGGATGACCGTCAGAAGGTAGGAGTCCGGGAGGCAGGAGGTCTCACTC
CCTGCCAGCGGCAGTGCTGACAGGATCCTCCTCAAAGGTCCTTAGCTTTGGCAACCCGGGTCTTCAAAGATGGCCTCGTCACCTTATATAGTCT
TCGATGGAGATCACATCGCTGGAGCTCAAAGGCTAATATCTGAGCCTATCTCCAACTGGCTGCCTCCACACACCTGCTTCCGTGATGGGATGTT
GGCAGCAGACACTGGTCTCATAGTGACATGAAGTTTTTTTTGTTTTTTTTTTGCTTTTTTTTTTTTTTTTTTTTTTTTGTGGGGTACATGTGTG
TGTGTTGTCTCTGTGTATATCCCAGAGAGGCAAGGCCAGACTTCATCAGCCAAGTCCCCAGCTGGGAGTTACCACCCTGGATGACAGCGCTGGC
TCTTTGGTTGCTCTGAGACCTCTTACACCCAGGTCACCACAGCTGGCTGGGAACTTACTGAGCAGTGGACTCTTCTGTGCCTCCTCTGAGTGAT
GTCTAGGAAGCATTATGTGTGTATGGGGCTGCCCACACCTTGTTTCTCTTGAGCTCTCAATCCTGGCACAGACATTAGAAGAACTGTGTTGTTC
CTCCACGATACAGGCCCAAGGGCCTGTCTCCCCCTGAAACTTAGGGTGGGTGTCTGAGCTAACCCCACTAGCACCGCCACACTAAGGACATCGA
GACAGGCTAAATGGGCTGTGACGACCTCCAGGTCATGCCTAAGCCACCAGACCTGTTTCTCTCCCACCTCGGAAGTGGCAGGACGGTGACATTG
TGGCTCTTCAGCCCTGGAGTTGAGACATAGGCACAGTGCACAGTGGGACCTTATTGGGACCTGTCTCAGCTCCGAATAGGATAAGCCCTGGGGT
AGGAGTTATGGCAGGAAGCTGACCAAGCAGTCTCTGCAGGAGAATCAGATTGTGGCCATAGCCTAGAGGAGGCTGGGGAGAGGATAGAAAATTG
GGTTTCCTATCTCACCTCTGCCCAGCACTGTTCAGAGAGGCACCGCCACCAGGCACCAGGCTGGGGAGGAAGAGATACCATGTAGGGAACACCT
CATCCAAAGAAAACCTTCCCAGGTGAGTTTGTGTAGTAGTGCGACAPGTGCATATGCAGGTGTCTACATATGTGTGGGTTCTGTCAGTGTAAGC
ACTTGGGGATATGTCAGTATATATTGATAGTCATATATATGCATGTGCATGGGTTGTGTGTGCACCTGTATGCCTGGAGCAGAGAGGGTAGCCT
GGAAAGACCAGCGGAATTTATGAGCTTGGCCACTCACACAATCCTTTCAGGGCTGGGGATCGGAGCCTGGGTTGGGATGGGTGAGCCCTGGGAG
TTACTTGGCCATGGTTCTCGTCCTGGCAATGGCAGCAGGTCAGATGGAGAGCCTGTCCCACACACAGCTTCATAAGGTGGCAGTGTGTCCATGG
AGACAGTGTGGAGTCTGCTCTGACCCTGGGTCCTCTGCTCTTGCTGACGGTGGCTCCAACTCTCGCCGCTGTCTGAGTCAAGGGCGCCATTCAG
CCTGGGACAGGAGTCAGTCATGGAGTAGGGCGGTGGTGCATCCGTGGGGACAIACAGCTGAGTGGCCCCTGGTCCCATGCATTCCTCATAGCTG
TGASACAAGAGTTTTCACTCAGGGGTTGTAGATTCTGAGACCTTCGGCCAGATGGAGGAGTCTGTATTCTSTATGGCAAGGAGCTGCCCTCATT
GTAGAATACAGCCAACTTCATATATGCTGGCTCCCATGAGACCCTCTAGTCCTCAGAAGGACAGTCTGACACCCTTAGCTGGGTAAAGACTTGA
CCCTCACATCCCATGTGAACCAAGCAAGTCCTTACACCCCTGCCCCTCTCTGTGGGGTTTTAGATTCCCTGGAGAAAGTAACAGCCCTGTCTAG
GCAGGAGTCCCAGCCTGGGCACTGTGAATGGGCTCGGTAGCACCCACTGAGACATCTCCACTGCTGTTTTCTAGATTTAAAGATGAGTCTCTAG
CCCTGAAACCAT
MOUSE SEQUENCE-mRNA
ACTATGCTGCTGCGGTCGCTGCGTAGTTGGGCTGCCCGSTCCCCGCGTAGCGTGGGCCCGGGGAGCTCCGGGAGCCCCGGGACCCTCGATTCGG
GCGCCGGGCCCCTGTGGGCACGTCGCCGCGCCTGGCCTCCGGATAAAGATAGAGAAAATGATAAGGAGAAAAAAGCAGTGGTTTGTATTGAGGG
CAATATTGCAAGTGGGAAGACGACATGCCTGGAGTTCTTCTCCAATACAACAGACGTCGAGGTGTTAATGGAGCCTGTGCTCAAGTGGAGAAAT
GTCCATGGCCATAACCCTCTGAGCCTCATGTACCATGATGCCAGCCGATGGGGCCTCACACTGCAGACGTACGTGCAGCTCACCATGCTGGACC
AGCACACGCGCCCCTCAGATGTCACCTGTACGGTTGATGGAAAGGTCAATTTACAGCGCAAGATACATTTTTGTAGAAAACCTGTATAGAGGTG
GAAGATGCCCGAGGTTGACTACGCGATTCTGTCTGAGTGGTTTGACTGGATCGTGAGGTAAATTGATGTCTCTGTTGATCTGATAGTTTATCTG
CGAACCACTCCCGAAATCTGCTACCAGAGATTAAAGATGAGGTGCCGGGAAGAGGAGAAAGTCATTCCGATGGAATACCTCCATGCTATTCACC
GCCTCTACSAGGAGTGGCTGGTCAACGGGAGCCTCTTCCCAGCTGCAGCCCCTGTTCTGGTGATTGAGGCTGACCACAACTTGGAGAAAATGTT
AGAACTCTTTGAACAAAACCGGGCCCGGATATTAACTCCAGAGAACTGGAAGCATGGACCCTAGGACTGAGTGATCTACAAGACAGCGCCAGGA
ACAGCCCAGCCAGGGCCAGGTTAGCTGCTAGGAGCATTTGGAAGAATCTACTCTTAGGAGGGTCTGTTATCTAATCAGATCTATTTTCTCAATG
GTCTTTTTCACTGCAGTACAAATGGCTTTTGTCTGTCAGATGCTTGCAGCATACATCCCTCCTGGTGCCCCTCCTGGTGGCTTCCTAACGATGA
AGCCCTGTGTTAATCACTCACTGTATCCCTGGCCTTGTTCTGGATCCTCTGCATCAGGTAACCTCATAGAGTGAGGGGAGAAAGTCTTCACCCC
AGGCAAGGATTTGAAGCCCAGAGAGGTGTGGGCACTCGTCAAGCCAGACATTGTGGAGGGACCTGGGACCATTCAGCTACCTGGTTTGGGCTGC
CTCCTCCTAAGCTGTTCCCCACCGCCCTGCTGCTCTGAGCACCAGCCCCCGCCTCTGTGAGTAGGAGTGGACAGTTTGTGATTCCATCATCTTT
CAGTACCGGGCCAGTTCAAGGACACCAGACATCACTAGGTGTTGGCACACCTCTCTTATTTGGCATGAGCCCAGGGTGTCCTGGTATGAGGGTA
CTGTGACCTCTATCCATTCCTTTGTCACCTGGACCTCGGTGTCCACTGGGGACTCCTGGATCGGCCTCTGCTTGGTGCCAGAGTCCTTGAGGAT
GAGTCCTCCCCAGACTCTACCCGTTCCCTGTCGTTTTGTGTCAGAATAAAGGGGGTTATGGGACCTGGACACTTTACCCTTTGCTGAGCGAGGC
CAAGGGCAGCCAGCTGAGCTTCCAAGACCCCATCACTCTCTCCTGTCCTTGTGTGAGAGAGAAGTGTCCCCTTGCTCCAGGGAGAGTGACAGTG
GCCTTACTCCTTTGAAGTGTTTTTCCATCTGAGCAGATGTCATGACCCACGAGCATGAGAAGTCATCTCTATTGGATCAGATAATGTACCTTAG
CTTTGCTGAGCCGTAACTGTGCACTGAAGGTGCAGCTTGTTACTTGAATCAGTCATGACCAAGAGAGGCCGGCCATTCCTCAGGACCGTGAGAC
TGGAAAGTCATGGTTTGCCAACAAAGACAATTTAAGCCTTGCTGCTCTTGACATTCTAGAATGGATGGAGTGCATCTGGAAAAGACTGGACACA
GATGATACAGGGGATATGATCACCGTATACCCTGGAGTAGCACAGCACAGGCTGGGGAGGGGGCTGCAGAGAGCCCTGCTCCCCTGTTTAGCTG
CCTGGGAGCTAGAAAGACGGCCAAGAGTCCAGCTCTTGGTCCTTTCTGTTTGGACCAATTCTTGGCCCTTTTGGTAGTATGAAGAGTAGCATCC
AGGGAGATTGTCCATCAGCACCTGCAGTCCAGCAATGGATAAACCTGAGAAAGGCAGGAACTTTGAACCAGCAGTACTCATGGGGGCATCTTTA
GAATTTGCAAACGAACAGTCTAGTACTTGCCCTTTGCTCTACCAGAACACTTGTTTTGGGGAGAAATGTGCTGTTTCTCTTTCTCTCTAGTAGC
CAATAAAATTCTACAGCTAAAAAAAAAAAAAAAAA
MOUSE SEQUENCE-CODING
ATGCTGCTGCGGTCGCTGCGTAGTTGGGCTGCCCGGTCCCCGCGTAGCGTGGGCCCGGGGAGCTCCGGGAGCCCCGGGAGCCTCCATTCGGGCG
CCGGGCCGCTGTGGGCACCTCGCCGCGCCTGGCCTCCGGATAAAGATAGAGAAAATGATAAGGAGAAAAAAGCAGTGGTTTGTATTGAGGGCAA
TATTGCAAGTGGGAAGACGACATGCCTGGAGTTCTTCTCCAATACAACAGACGTCGAGGTGTTAATGGAGCCTGTGCTCAAGTGGAGAAATGTC
CATGGCCATAACCCTCTGAGCCTCATGTACCATGATGCCAGCCGATGGGGCCTCACACTGCAGACGTACGTGAAGCTCACCATGCTGGACCAGC
ACACGCGCCCTCAGATGTCACCTGTACGGTTGATGGAAAGGTCAATTTACAGCGCAAGATACATTTTTGTAGAAAACCTGTATAGAGGTGGGAA
GATGCCCGAGGTTGACTACGCGATTCTGTCTGAGTGGTTTGACTGGATCGTCAGGAACATTGATGTCTCTGTTGATCTGATAGTTTATCTGCGA
ACCACTCCCGAAATCTGCTACCAGAGATTAAAGATGAGGTGCCGGGAAGAGGAGAAAGTCATTCCGATGGAATACCTCCATGCTATTCACCGCC
TCTACGAGGAGTGGCTGGTCAACGGGAGCCTCTTCCCAGCTGCAGCCCCTGTTCTGGTGATTGAGGCTGACCACAACTTGGAGAAAATGTTAGA
ACTCTTTGAACAAAACCGGGCCCGGATATTAACTCCAGAGAACTGGAAGCATGGACCCTAG
HUMAN SEQUENCE-GENOMIC
CAAACCTCACTCTTGCCCCAGCTCTTGTTCACAACTCAATACCCAACCATTCAGATACACCTGCAGGTCCTGGAGGGTCATGCTCTTTCTCCAC
CTAGACCCTCACATATGCTGCTGCTTTTCTAGCAGAAAAGCACTTTCCCTTCCCTGTATCAGTCATCTGCTAACTACGTTTCATCCTTCAGCCC
TCAGTTTGGGCACTTTTTCTTCACAGCCTACTGTGACTTGGTCCCTTTCCCTTGCTGGGTTGCATCCACGCCATCCTAGGTGTATATCTCTCAT
AGCACCTATATTCTCTGTGTTGCTTCCTGGGTGCATTTAAAGTTTCTGAGGTACAAGGTTTTTGTTTTGTCTTAACCAAAGAACCAATTACAGA
ATCTAGGATTAGCAAATACTTAACATTTGCTGAATAAATAATTTCATTAGATCAAGATCAGAAAGGATTCTGGGTTTCGTAATGGAGCAAAAAC
TTAGTTACCCATATAACACCCCATTTCTAAGAGATACAATATCAGTAAAGTTTGATCCTTAGAGATTTGGGGACTATTCCTTTCAGTGTGCTAC
ATATAAGCTACTCTCTCTCAGTGTGTTAGTTTTATTAAACTCTAGAGACAGAGTGTGAAAAGCACTATACAAGACCTAGAGGATGTGGACCAGT
AAAAAAGAAAGGCTAAAATGTGTTAGTTTTATTAAACTCTAGAGAAGCAGAGTGTGAAAAGCAACTATACAAGACCTAGAGGATGTGGACCAGT
GTTCATTATCCTCTTGAAGAAATCTTTAGAAAAATTTAGTTTAACCCTTTCTGCCACTAAATGAGGGCCAACTGAGGAAGACTTTGTAATCTCT
TCTCCTAGACCTCCCTCCAACGAAGTCCAGCTCTGAAGTGTACATCAAGTAGTTTTGAGAGGAGAGGTTCTATGAAGATCCTATTTCCCAGCTT
TTCCCTTTTAGGACTTTTAAAAAGCTTATGATTTGCCTGAAATGCTTGCCACTGGCCTTGAAGGTGGTTCCAGAAGGTACCATTTAAAGGCATG
CTTATGGTTTAAGAATAGCTCTAAAGTCTTTTCTTGTTAATAGGAATTATCAGCTTGGATATTAGCACTGTGGCATCCAAGAGGATGAGACCAC
TGCAGCCAAGGAAGACATGGGCAGCAATCAGTCCAGCCAAAGAGGAGAAGGGCAGCAGGGGATTCAGGAGGGAGACCTACAGAGACAGGATGCT
GGGAATGAGACTTGCAAGAGGACAGAGGAGAAGTCACTCAGTTTTTCTTAGAATCAGCTTCTAGTCCAGGCTGACGGGCTGCATAGATCTTATC
GAATAACTTCAATCTGCCCAATTTGACAGTTTATCAGAACCATCTGGGCATTCCACTGCGCAGTTATTCCTAATTATATAGTATGGCAGGTTAC
AGTCTATATACTATGAAATATAAGAGGTGCCACATTATTTAGTAATAACTACAGATATTTTCAAGTGACATAAACATTGTACTTPAGCCTCCCC
AATTAGTTTGATTTTCCATTTAGTGACCAAAGATGTGCAAGGATAGATACTTGTGTATCTGTCTCCATCTCTATCCCAAATTTTGAGGCATATT
AACATGCATCCAAATAGGTAATAAAGATTAAGAATGCACTACAATTTTGTTAACATGGTCCTAAATTTAAATTTCTTTAGAAGATCTATCCAAG
TGAATTCTAACAGAAGCGTCAGCCACATCTACTGATAGATGGTGCAGAGGACTTATGCTCTGTGGCTGCCCTGCATCCTTGAGACCCTCTCCCA
AAGCAATACACGGCACATTAGCAATGGGTAGAGCTCCTCCTTCCTAAGCCTAACAGAGATTACCTTAAAGGTTTAAGAGAAGCAAAAAACAGAG
AATGACAATTTCTAAAATAATAGGCTGGGCACAGTGGGTCATGCCTATAATCCCAGCATTTGGGAGGCTGAGGTAGGAGGATACGCTTGAAGCT
AGGAGTTCAAGACCAGCCTGGGCAACATAAGGAGACCTCATCTCCAAATAAACAAAAATAACCAGATACTGGGGTTTCTGCCATATACACACAC
ATATAAAACAGCCATGCCTAAAGTGTATGATATTGACTTCTCTTTGTCTTTTAGAGGACTGGGTATTCCTCTCTGTTTTAAGTCCAATTAATTA
TTCCTCTTGTGCTTCTGACACTATCACTTCCCACATCAGTTCTCTTGATGTTTTTCCACCAAGTACCTCCGTTCCCCCTTGTACTTTCAATCTG
TCCTCTGCTAGCTCCTTTTCTCATCATCCTAGAAACATCTCAGTTGTGTTCTTTTAAAATACACACACACATACTCCTTCCTTTTATCCTGCTC
ATATAAAACAGCCATGCCTAAAGTGTATGATATTGACTTCTCTTTGTCTTTTAGAGGACTGGGTATTCCTCTCTGTTTTTAAGTCCAATTATTA
TTCCTCTTGTGCTTCTGACACTATCACTTCCCACATCAGTTCTCTTGATGTTTTTCCACCATAGTACCTCCGTTCCCCCTTGTACTTTCATCTG
TCCTCTGCTAGCTCCTTTTCTCATCATCCTAGACATCTCACTTCTGTTCTTTTATAAAATACACACACACATACTCCTPCCTTTTATCCTGCTC
CCCACAGAAACCCTTGTTCTGTTTTCTACTGTTGTTATGCTTCTTCAAAAAGTCTGTATACTGGCTGTCTATAATTTCTTACCATCCATTTATC
ACTCCTTAATTCTTAACAATCTGGAGTCTACTGTCAGATTTAAACTTAGAATGCTGCCATGAAAAATACTGAGGAGCAAAGTTCAAAACAAATT
GTCAGGGTGGGCGCGGTGGCTCATGCCTGTAATCCCAGCACTTTGGGAGGCCAAGGCAGGCAGATCGCCTGAGGTCAAGAGTTTGAGACCAGCC
TGGCCAACATGGTGAAACCTTGTCTCTACTAAAAATACAAAAATTATCCAGGTGTGGTAGCAGGTGCCTGTAATCCCAGCTACTCGGGTGGCTG
AGGCAGCAGAATCGCTTGAACCCAGGATGCGCGGGTTGCAGGGAGCTGAGATGCCACTCCACTCCAGCCTGGGTGATAGAGCTAGACCCTGTCT
ATATAAAACAGCCATGCCTAAAGTGTATGATATTGACTTCTCTTTGTCTTTTAGAGGACTGGGTATTCCTCTCTGTTTTAAGTCCAATTAATTA
TTCCTCTTGTGCTTTCTGACACTATCACTTCCCACATCAGTTCTCTTGATGTTTTCCACCAAGTACCTCCGTTCCCCCTTGTACTTTCAATCTG
TCCTCTGCTAGCTCCTTTTCTCATCATCCTAGAAACATCTCAGTTGTGTTCTTTTAAAATACACACACACATACTCCTTCCTTTTATCCTGCTC
CCCACAGAAACCCTTGTTCTGTTTTCTACTGTTGTTATGCTTCTTCAAAAAGTCTGTATACTGGCTGTCTATAATTTCTTACCATCCATTTATC
CTTAAACCTTTCTATGGTTTCTGTGAAACTGTGTTCTCCTGTGATTTATATTTACAGTTCTTACATCTTTCCTAAGCTCCTGGCCTTTGTTTCC
AGTTATCTTTTACATATCTCACTAATAATATTTCAGACATTACCACTCTATGGATATTGCTTTTGCTAACATCAGTGATTTCAGAAATACTAAA
TTTAATGAATGCTTTTTTTCTTTTTTCCAGTACTTTGAGCTGCTGTTTTACAATGAACATTTTTCATTCCTTATCCTAGTGGACCCCTATGACG
TTATTGCCATTGGCTGTCCTTCCCCTACTAGTGTTTTCTTTTGGCTCCCGTGTCAGTTCTCCGAGATTTTAAACCTCCGCGACTGTTCTCTCTT
GGTGGGTTCTTCTCCTTTTATTCTCCCCTGTAAAATGCTGTCATTCTCATTCCACTCAATGAGTCTGGGTCTCTAACCCAGGTCCATTCTCCAC
ATTTCACACCAAGTAGCCATTTAGAATCCCTATCTGGATAACCTACAGGAATCTCAATTCACCATGTCTAAAACTGAAATCCCGCCCCTTCCTC
CTGGATTCACAACCTCAACTGGTGGTACCACAATCTACCTTATCACCCATGCCAGGAACCTAAGATCACTGTTCCTACCCATTCCCCAACTGAT
CAATAATCGGGTCCTGCTAACTTAATGCCTAAACATTTTGGGCACTCCCTCCTCTTCATCCTTACAACTACTGCCTTTCATCGTCTTTTCCATG
AACTATCATGATGAAATTTCTAACATGCAAATTTGTTTACGCATTTCTTCATCTATTTAAAATTCTTCAGTGGCTCTCCAGGGTCTTTAGAGCC
TGCTTCTCACCATGTTTTCAAGATCTTCCACAATTTGGCTCCTGTCTACTTCTTGAGAAAAGACTTGCTTCACAATTTATCCACATGAATCAAA
CTCCTGATCCTTTTTTCACATTTGCTTTATCAATAGGCTGTCTCATCTCAGTGGATGGTGACTCCATCCTTCCAGTTGCTTAGACCAAAAACTT
CAGAAGCTGTCTTGGACTCCTCTTTTTCCCCACATCTGGCTCATCAGCAAATCTTGTTGGTGCTACTTTCAAAATATTACATCCCAAATGTGGC
CACTTCTTATTACCTGCTTTGCTACCACCAGCCACCATCATCATTTCTTGCCCAGATTACTGCAATTGGTCTCCTTGGTTTTACCTTTGCCCCT
GCAGCAATCCCCTTCTGTCCTTCCCTACCAATCTACTGTTATAATAGCAGCTAGTATAPTTTTGTTTGTTTTTGAGACGGAGGCTCACTTTGTC
ACCCAGCTGGAGTGCAGTGGGGTGATTTTGGCTTACCTCAACCTCTGTCTCTCCGGTTCAAAGTGATTCTCCTGCCTCAGCCTCCCGAGTAGCT
GAGATTACAGGCATACGCCACCATGCCTGGCTAATTTTTTTTGTATTTTTAGTAGAGATGGGGTTTCACCATGTTGGTAAGGCTGCTGGAACTC
CTGACTTCAAATGATCTGCCTGCCTTGGCCTCCTAAAGTGCTGGGATTACAGGGTGAGCCACCGGGCCTGGCCTAGTATACTTTTAAAATGTAA
GTTAGATCATATACTTTACTTAAAACCTTCCTCATCTTCACTCACTCACCCCAGTCCAGTTACGCTGGTTTCCTTGCTCTTTCTTGATGCCTTC
CCACACATTCCTGCCATAAGGCCACTGCACTGGATTTTCTCTGCTTCAAACACTCTTCTTCCAGGTATTCTCATAGCTAACTCCCTCATTTCAC
TCAAATCTTTGATCAGATGTCATCTCAACAAAGCCTAGAATTCTATTCATTACGCAACTTTTCTCTGCAACTCCTTCCCCGCCTTACACTCCCT
TACCTGGCTTTTACTTTTCTCGTTCCACTGCACGTACCACCTTCTAGAATACTAAAATTTATTGTGTTTGCTTCCCCTGCCCTCCCATCCCAAT
ATGTGTACTCCACGAGGGCAGGGATCTTTGTTTTTTTTCACTAGTATTTTTCTAAGAGCCTGGAATAGTGCCTGGCACATGACAGGCATTACAC
AGATTATTTGTTGAATAAATGAGQTAACACTCAACTTCTTATATTTCTCTGCATCAATTGTCCTATTTTTTGCCTCCATGCCTTTACTCAGGTG
GCTCTTAGATCCAACCCTAGATTGCTCTAGGAAGAGCTTTTTTCTGACACCTCTCCTTGGTTAGACGCCTCGCTTCTGTGTTCTATCTCCATCT
TATCACATATCTTAACTATCTGCTCATGTGTCTGTCTCATAATTAAACTGAAGAACTCCATGAGTCAGGGATTTTTGTCCTGGTTCGTCTTTGC
TGTTCCAGCTCCTAGCCCAGAGTTTGGACCATATTAAGTATTCAAATGGTTTGTTGAGAGAATTAATATATCAAAAACTAACCATTCTCACAGG
ACCATTCTCACATGGTTCCCCCTTCAGTGTTCAAGATCTTTCCTTTTTCAGTGTTCAAGATCCCAGACCTTCTGGTCACTTTTGCCCTGAGTAT
TTACTCCCAATCAAAAGTTATTTCTCCTTCCCTCAACTCCAGAGTGGTTTCTAAACCTCTTATGTATCTCAACTTACATGATATGAAAGCTACT
TCTCACAATCACACACTTTTGGTGTGAGAAGGATCTTATATGCACTTTGTTCAACCCACTCACCTTAAGTTTGAGTCTCCTTTATAACATCCTC
ACCAAGTGCTCACATATGCCTGGTACTTTTGTCTGCGAATGTACTGCCTTCCCGAGTGGCCATTTAGTTTTGGGAGCTCTCCCAATATAAGATT
CCTCCTCACATTAATTGGAAATGTTTTCCTTGTAACTTTGAACTATCTCCATTTGCCTCTCCCATGTCAACTCTTTAAAACTTAAAATGATTTT
ATTTTTTCCTTCCCTGTAAGGTTTCTCTGTGAATTCCTCCTAATGATGAGTTTCTAGATTAGATTCCTTCACTAGGATAGTTTCCACTTCTCTA
ACTAGCTAGGTCACTTCTCTCCAAATGATCAACTTCCTCCAAACCTGTTCAGAAGTGACTGCAGTGCTCCCGGTAGGAAGTCTGCTCCCGGAAA
CGAGCAGTTGTCTCCTTGTCCTGGTTCTGGGCTGTATCAGCCTTGAACTCAGTCACATCAAACGTCTGTCTCACGGAAGTCCACACAACATTCA
CCGAAACCCCTGAGGCTTTTTTTTTTTTCTTTCAAATGTTGTGGGCTTCTGAGAGGTATAACTGCAGTTGTCTCGCCTGGTAACCTGGGTACCC
GGTTTGTGGGTCCAAATGTAGACCATTTGGGTAGATCTGATTTTTAATTTAAAAAAAGGGTTTTTTTTTGTTTTTGTTTTTAAAGAGGCAGGAC
CTCTCTGTCGCCTAGGCTTGTGTGCAGTGGTGCGATCAACGTTCACTGCAGCCTCGGAACTCCTGGGATCAGCGATCCTCCCGTCCCGGCCTCC
CAAAGTGCTGGGACCACAGGTCTGCGCCACGGCGCCCCGCCCTCTCTGGTTTTCATGATGAGTGTGTCCCTAAACCCTCCTAAACGGGAGGACA
GTGCCGTATCTCTCTCCTTCTTCCCCAAGGAGGCCATGCCTTGCCTTTCAATCTTGATCAAAGCGAGGTTGAAAAGCAAACAGGCGTTCAGCTT
TGCACTTCACACCCACATCTCCCGCCCCCCAGCAGCTTCATCAGCCTCCCGCCCTCCGCGCCCCGGCCTCACAGCCGATAGAATCTTCACGTGG
CCTTTCACACTGAAGCAGAAGGGGCGATGTTTTATTTTCGGCTGCACGTTATCCATCGCGTCTGCAGACCCAGCAGCAGCACTTTCCCTCAACT
CTTCTCAGCTGGCTGCCTGAGTAGGTTCTGCGAAGCGATAGCAACCGCCACCGCGGCGGAGCACCGCCCTCCCCTACTTCTCGCCCAGCTCGGC
TTCCCGCTCTCTTGCGCGCATGCGCACGGCGCCGCGTTCGCTCTCGCGCATGCGCCCGAACGGGATCCCGCGGCCGTCCGCTCTGGCTTGTCCT
GTCTTTCAGTTATCGCGTAGCACATTAGCGGACCTAGAGCTGGGCACATATTAGCGGACCTAGAGGTGGGCACGAAGGCCAGCGCCCTGTTAGC
CCGCCGTGTTCTGGCCTGGCGTTAATGACGGCAGCACTCCAGCTCTCCTGAGGAGTCTTCCTGTCGGGAAATCCTCCCAGGGTTGGGAGCAGAG
GAGAGGGAGGGGGCTGTAGAAATGGCGGCCCCATCTCCCAACAACTTGGGCATTGTGAATATCACCTCCTTAAAGGGGATCTCCTTTGGTCATC
CCGTCTAGAGCAGCCACCATAACTTCTGAGCGTTTATTGCTAGCTGATATATATCAGAAAAATACAAATTCCACAAAAGCAGGGACTGGTCTGC
TTCTCTCCCTGCAGGGCCCAGGTTCTGGCACATAGTTGGTGCAGAAAGTGTGCAGCCTCAGGTCCTATCCAAGCCCCCAGGGCATCACACTCGG
GACTTGTTCTGCATATTTTTACTTTTGCCTCCCACTGGTACTAGTTCTTCCGTGGAACAGCCTGAGTCCCTTCAGATACTTAATGTTTTTTCTC
AAGTGCTGCCATGAAGCCAGATCTCCACCGTCTTGGGGCATTCCTTTTTAGGGATGGGPAGTATATGTCGCTCCTTTTATGTGATTTACATTCT
ATCTTGGATAATTTGGCCATCACCGTAGTTCATTCAGATCTGTTTGGATCCTGCCCATCTCAGCTTCAGTCCATTTCATTCTTTTAAATCTGAT
CGACAGTTACCTCCAACAGCTTCATCACAAATCACTCACAAAAATGGCCTTAATCCTGAAGTTTATTTACGGAGAGCACACTTGCTAGGTGTGT
GGCAGATATACAGGAAGCACAAGATGAGGCAGCAGATCTAGAGGCAAATGACTTCCTTCTCCCTGCCTAGTGGTGACTGCCAGCATCACGCCCT
CCCGGGAGAGGTGAGAAACCCCTCCACGCAAGCACTGGAACCTTCACAGTCAAGAGTGGCAACAGCTCCGGTTACTGAACTTGGGCCTGTTGAA
TTCTAATACTCTGTGACTCCACATCTGGGCTGAATTTTTGCTGAGTATGATCGAATTTACATGCTTCCPCCCTAGCCCCTACTTGTCTGTATAG
TTGGAATATTTGGTTGCCTCCTCTGGAGGGATCTAGTACGTTTAGAGTCTAGACGCTGGAACTGTCAAAGTTCAGAGGAAAGAGCTCCAGCTGC
AAAGCAAGAGAAATGGGCTGGAATTCTAGCTTCACCCCTTAATGAATGCTTCTGATTTTTTTTTTTTTTTTTTTTTTTGAGACGTAGTCTCACT
CTATCGCCCAGGCTGGATTGCAGTGGCCACGATCTCAGCTCACTGCAACCTCCGCCTCCCAGACTCAAGCGATTCTCGTGCCTGAGCCTCCTGA
GTAGCTGGGATTACAGGCGTGCGCTACCACGCCCGCCTAATTTTTGTATTTTTAGTAGAGACACTTTTTGGCCATGTTCGTCAGGCTGGTCTTG
AACTCATGACCTCAAGTGATCTACCTTCCTCCGCCTCCGAAAGTGCTGGCATTACAGGCCCGAGCCACCGCGCCCAGCCGCTTCTGATCATTAA
AAAAAAATTTTTTTTTTGGCGGGGGGAACGTAGTGTCCCTCTGTTGCTCAGGCTGGAGTGCAGTGCAGTGATCTCGGCTCACTGCAATCTCTGC
CTCCCAGGTTCAAGCCATTTTCCTCCCTCAGCCTCCTGAGTAGCTCGGTATACGGGTGCCCCCCACCACACCCAGCTTATTTTTGCATTTTTAG
TAGCCATGGGGTTTCGCCATGTTGGCCAAGCCTGGTCTCCAACTTCTGCCCTCAGGTGATCTGCCTTCCTTCGCCTCCCAAAGTGCTGGGATTA
CAGGCGTGAGCCACCGTGCCTGGCCAAAAAATTTATGTTTTAAAAAGACTAGTCAAGTGCAGTAGTGAGAAGGGGGGAAAGAGTAGAGCAAGGA
GTTATATCTGTTGCTTCTGACCATTTTGAACAAGTTACCTAATTCTCTCAGCACAAGCTCGGAGAATGGGACAGACACTTATCTATTTGCAGGG
TTGTTGGGAGGAATAAGTGACATCATGAGTGTGTGCCAGGTGTCTGATTACAGAAGGTGTTCAATTAATCTGCAATCATTAATTAACCCTTCAG
TCGCTGGTATTATTTGCCATCCATCCTCCGAGTGTTGCCAAGTTATGGGTGCGTTCTGCCAGCGTCCTAGCAGTGGTAAGGCTTCTGGCTGCCA
GCGGCGAACCTCTCCCTTCGAGTATTTCTCCTCTTGCTGAGATGAAATGCGACCGGGTCTCTTTAAGGGCCAGGCGCCGGGATCCAGGCGGCGC
CCAACGGCTGGACTAGCAGTCGTCCGCGCCGACTCGCACAAGAAGGAACCCCGGGCCTCTGGATCCGCTCGCCCGGCTATGCTGCTGTGGCCGC
TGCGGGGCTGGGCCGCCCGGGCCCTGCGCTGCTTTGGGCCGGGATAGTCGCGGGAGCCCGGCCTGGGCCCCGGGCCGCGGAACTGCTAGCGCCG
GGCCTGGCCTCCCGGTAACGCGCGTCTTGGTCCCGCCTCCCAGCAGCCCCTATGCGCCCACCTACTCCCCGCCCCTCGGCTTCCGGAACCCGCC
CGAGCCCGAAGCGCCTCTTCCGAGGCGCGGGATTTCCTCCCCGCCTGCGGCTGGGACGGGAACGGCGATCGGCCGTTACCCGCCCGCGAGACCG
CAGCGAGCGCTGGGGGCGCTCGGCTTTGGTTCTGGGATGGAGAGAGGGCCTTTAAAGAGCAGGACGGACACGCTCTGAACATTCCGGCCCAAAC
TCGGTCCCCCGACCCATTTCCTGTCCCGATTCTTTCGACCTTGGGGGCGATGGGGACTGGGAGCTGCCTTAAGCCGATTCTGCTCACACTCTGG
TGCCAGCGTGGGGGAGGGGGGTTACGCCCCCGGCGCTCTCCTGCCCTCCCTGGGAGTGGGGACCCACACTAGAACGGGTCACTGTTACACCCGG
GTTCTGGTGTCCGCCATATGGCTGTTGAGAGACGCCTCAGAACCACCTGTGGGACGGATGGGCAGCGATGCCCGCCCTGGGCCTAGAGGAAAAC
CTGGGTCCCAGGCCCAACCACGTCCGCCCCCCACTCTTCCCCGTGCACCGCGAGTGGAGGCTGTTCTGCCTTTCCACTTGCTTAACATCGATAG
TCTGTGAAGATCTAAATAAATAACCTGGAAAATGCAAAGTATAATGTACTAAGTGAAAGAACTCGAGCACACTGGGGGTCCACGAAGCTGCAGC
TGCAGCCCAGGGTGGCACATCGGCGAGACGGTGGTGCGCGCCTCCAATCTCGGAGGGCGCCCAAAATGGAAAATGTTTTTAGGCCAGGGAGTGA
GCATAAACATTAGTGTTTTTGTGGTCATGTAGCAAATTAGGGCAGAGGAGAGTTATTTAAAAGTGAGTTTAAAAACTGATTTCCTTTCTTTTGT
AGATAAAGAACAGGAAAAAGAGAAAATATCAGTGGTAAGTCCCTCTTTTATGTGTACTCTCTAGGATTAATTTGGTTTTTTGAGAGGAGAGACA
AAAAAGCAGAGTAAAAAGCAAAAATACATTCCATAGTAAAAAGCAAAAATACAAGAGGATCTCCAGGGAGAAGGAAGTCTGTCACCCTCCTAGA
AGGCCACCTCTGTTACCAATTTCTTGTATCTTTCCCCAGAGTCTCCACAGATACAACCACACGACTATTTGTATCCTTCAGGTAGATTTCGACT
GTTAAAGCTTAAGGGATGATTGTGAAAGGATTTGAAAAACGCTCATTGTGTTGTGCGGAAGGTCAGTTTCATCTTGGGTTGGGCAGCTCAGATG
TGAGGGAAGCTCTGTCCACCACCACCCTCTCCCCCCAGTGTTGAGACTGGTGGGTGGGCAGGGGATCAGAGAGAAACCATTAGGGTCCACAGAT
GTGGGTTCAGTTATCAGGGTGGGTTTGAGATGCTGGCTCTGGGCTGGGGCTGTGACTAGTGTGAGGCTGAAGGTCAAAGAAGGGTCAGGTTGGT
GTCCATGGGGATATTGGGTGCTTTAGCTGGCTGGGGTAGGGAGTGATATTTTGGTCACAAACCAAGGCTCAATTCCTTAAAAGAACTGAAAGTC
CCTAGCTCCAGTGGCTGGGAAGGGGGTGGCTTGGCAAGTGGTGGCAGGAGTACCGTGGCTGGGATCTGCAGAGGGTAGACAGTGCGGGCAGGCC
CCCAGCGTACTCAGGGTGGTGGAGAGCTCTTGGGAAGTAGTGAAACCACATAGAGAGGATTGAAATGCTTGAGATTTGTGGAAGTTTGTTGAAA
TTAGGAGAAGGATATGCCAGCGGGTGCCATAGAAGGGGAGGGAAATGCAAAGTTGAGCTGGATGGTAATTAATGTATTTTGTTTGTTTTATACA
TTTAAGAGACCGCGTCTTGCTGTGTTCCCCAGGCTGGCCTCCAACTCCTAGCTCGAAGTGATCCTCTCGCATCAGCCTCCTGAGTACCTGTGAT
GACAAGTGTGAGCCACTGTGCCTGGCTATTTATTAGTTATTCAGCTAGCATTGATGTGAGCTGGGCCTGTTCTTTGTGCTTTTTGGGTGTAATT
TGATGTGCCCCCAGACACACAATAGCTAGTTCATAGAAAGTGGTATGGAGCCTGCATTCAGGCTCAGGACTGTTTGTAAACATTCAAGTCCTGT
GAGCTCCTGTCTGGGATGCACTGGGCTAGGACCTGGGCACCTGGCAAACAGCCCTGACATTTGGGAGTCTGCGGTCTGTCTGATGGGGGAGGTT
TGTCTGATGGGGGAGGTCTGTCTGATGGGGGAGACAGCAGTAACTAGGAGAATGGTGGAACTAGAACAGCAGTGAGTATGGTGGCTGTGAGTAG
GGGGGCTGATGACTGAGAGCAGGTTTGGGAAGCCTTATTGCCTGCTGGGAGATGGAGCAGAGTTTGAGGCAGGAGTGTTGGAACAGGTACAAGT
CACAGAAGAATGAGAACCTGCAGGCCTGGGCTTGTGGCCAGTCCCTAGAGAGGTGGCTGGGAGGGCTGGGGGATAGGTGCTAGAGGGACTGAGA
ATAGGTCCTAAAACCCAGAGTCACTGAAGGGCCTCACCCCCACCCAGCACGATCCGGGTTGCCTCTCAGGGAGCGATGTGGGGAACCATGAGTG
GAGGGCCTAACTGGGTGAGACTGGAAGGGGAGAGACCTTAACTAAACTAGAGATGGCACAGAGCCCGCAGCTGAGTGGGAAAGCAGGGCCCAGG
GGAGTTGCTGGTTGGTTGACTGTTGTTGGTGGGTGAGGAGAGATGTCCAGAGTGTTAAAGGTGTCTGGGTGGATGGTGCAGCCATTACATGGCA
CAGTTTACAATATTAGATAATTGAGTATCACAACAAAGTTTTTTAATAAAATAACTGGACAGAACTTAACATCCAACAGGGCCATACCTAATGG
CTCATGCCTATAATCCCAGCACTTCGGGAGGCAAAGGTGGGAGGATTGCTTGAGCTCAGGAGTCTGAGACCAGCCTGGGCAACATGGCAAAATG
CTGTTTCTACAAAAAATACAAAAAATTAGCTGGGCATGGTGGCATGCACCAGTAGTCCTAGCTACTCGGGAGGCTGAGGTGGGAGGGTTGCTTG
AGCCTGGGAGGTCAAGGCTGCAGTGAGCTGTTATTGCATTGCTGCACTCCATCGTGGGCAACAGAACGAGACCCTGTCTCAAAAAAAAAAAAAA
AATCAAATAGTAGGGAGTTCAATAAATTGTGACAGGTCTTATATAATGGATTATGTAAAGCCATTAAAAACCATGTTTTTTGAAAATATTTAGT
GACCTGGGAAGGCACTCACTATGTAAAAGATGAAAAAAAAAAAATAACAAACCAACAAAAGTATATGAAGTATGATCCCAAATGAAAAAAAAAA
CTGTCCTTAGGAAACAAACAGGAAAAGAAAATATTATAAACATTAATGCCGAAATTCTCAGTCTAGTTAAGAGATAGATGTGGGTAACAAGGAG
CATGTAAGAGAGAGTTTGTGGTGTGATGGGGTAAAAGTGTCTTCCCTTAGTTATTTGAATGTTAAATGAAATTCAGCTGGGTTCCTTTTTAAAA
CAGCTTTATTGTAATTTGCATACCACAAAATTCACCCATTTAAATGTGCAATTCAATGATTTTTAGTAAATTTACAGAGTAAACAGCACTACAG
AGTAAACACCACTACAATCTAATTTTATTTTATTTTATTTTTATTTTTTATTTTTTGAGACAAGGTCTGGCTCTGTTGTCCAGGCTGGAGGACA
GTGGCACAATCACAACTTACTGCAGCCTTGACTTCCTCGTCTCAGGTGATCCTCCCACATCACCCTCCCTAGTAGCTGGGACTACAGGGACATA
CCACCATTCCTGGCTAATTTTTGTATTTGTTGTAGAGATGGGATTTCTACATGTTGCCCAGGCTGTGCAATCTAATTTTATAACATTTCCATTA
CTCCAAAAAGAAACCTTGTGCCCATTTGCAATCTCTTCTGGCCCCAGGTGACCACTATTCTACTTCCTGTCTCTTTAGGGATTTGTGTTTTCTG
GACGTTCCATATAAAATGGAACATATAGCGTCGGATCTTTTGTATCTGGCTTCTTTCACTTAGCAAATTTTTTTTTTTTCTTTTTTGAGATGGA
GTTTCTCTCTGTTGCCCAGGCTGGAGTGCAATGGCATGATCTTGGCTCACTGCATCCTCCGCCGCCAGGGTTCAAGCCATTTTCCTGCCTCAGC
CTCCCAAGTAGCTGGGATTACAGTCATGTGCCACCACGTGCAGCTAATTTTTGTATTTTTAGTAGAGTCGGGTTTCACCACGTTAGCCAGTGCT
GGTCTCCAACTCCTAACCTCAGGTGATCCGCCTGTCTAGGCCTCCGAAAGTCCTGGGATTAAAGGCGTGAGCCACCGCTCCTGGTTCCTTAGCA
TAATATTTTGACATGCATCTGCCTTGTAGCACGTATTAGTATGTCATTTTTTCCTATAGCTGATTAGTATTCATQJCGTGGCTGTAGTGCACTT
TGTTTATCCACTTGCQGTTGTTAGACGTTTCCGCAAAGTTTCTTTTGACTGCAGATTTCTCACAATCTTTGATAAGACAGGATATTTGGATTCT
CCCTGTGGAGGCTTTGGCACCCAAGCCAGTTTGCCTCTGGAACCCTGGGTTCTAATTAAACATCCAAATGACAATGATTGCTGAGATTTACGAG
TGATTTTTATTTTCTTCTTTTAGGGATTGTGTAATAAGGTTTGATCAGAGAAATCCAACTGATAAATACATATATGCATATTCATAAACATATA
CATATATTCATGCATACATACATTCTTGTGTGTGAQAATGTATTTCTTTTTTTTTTTTTTTTTCAAGACAGGGTCTCACTCTGTCACCCAGACT
GGAGTGCAGTGGTGTGATCTCGGCTCACCACAACCTCCACCTCTCAGCTCCCAAGCTCAAGCGATTCTCTTGCATCAGCCTCCCCAGTAGCTGG
GATTACAGGCATCCGCCACCACGCCTGGCTAATTTTTGTATTTTTAGTAGAGACGGGGTTTCACAATCTTGGCCAGGCTGGTCTTGATCTCCTG
ACCTCAAATGATCCCCTGCCTCGGCCTCCCAAAATGCTCGGATTACACGCGTGAGCCACTGCGCCTGGCCACTGAGTATGTATTTCTTAGCACA
AGAATGCTTAATTTCCATTTTTAGGAAAGATTAAATATTGTTACTTATAATAGGGATAGATGGCTCATCACAGTTTCAGTTATAATCATGGAAA
ATTGGACTCAAGGGCTCATAGGAGAGGAAAATGGAGATAAATTGTAGCGTAATCACAGAGTGTTGGGCCAGGCCGGCCATGTGCTCCCACAGCA
TGGGGACCATCTCCTTTTCACAGTTTCCTGTTGCTTTCCTCCACACACTCATGAGCTCTTTGGCCGTTGCAACGAGCCCCTGAGTACACAGTGC
CTGGCACAGCATAGGCCTTCAATAAACAGTTGAAACGTGCTCAAAGATGAGGACTGTATAGAAAATAATATTTGGCATGAAATATGCTCATATG
TCATGGGGTTTATCTCTTAGTATTGGGATTATGGGTGATAGTTTCTCCTTTTTGATTTTCTGTATTTTCTTCAATCGGCATGCACTTCTCTTAT
AATATTTTTAGACATCACTTTAAAAAGAAATGTAAAAGACTCCCTTTGCTTGCTTCCTGTTCCCCCTTCCATTCAGGGGAACTTTGTGAGATTC
TTCCTGCGTTGCCTGCTGTAAACCGGAGGGCAGATTGGCTGAGCTCATTCAGGGCCTGACATTCACCAGCTCTTCACGATTTQATGCTGGGGAT
AGGAACAAGTTTCTGGGAGAAGGGCTTATCTGAACAACCTGTTAAAATGCTTCAATGAACAGGAAAGAAAAAAGACTGTAGAGTGATGTCTAGG
TTGAGCTCTTTTTATATATCTAGGAAGAGGAAAAGTTTTCTTTAAAGAGCAATAAAAAAATTAAATCCATTAGTGCCCTAATGGGGTCTTTTCA
TGCCCTTTTGTTCATTCCCTTGGGTTCATTTTGTAGTTTGGGGCTCTAGAAGGAGTGGGCTGGGAGGGACCCAGGATAGAGAGTGGCCTTGTGT
CTCCTGGATACCCGTTAGGATCAAGGCAGGAGGAACCTTCTCCGCCAGCTGACAGAGTTCTAGTTGCTGGAGAGACTTTAGTGGGGTCGGGTGA
GACGGCCCACTGTGACCTGGTGTTTATGTGAGGTGTGACTGAGGCTCCATCAGGAGTCGTGAGAAGGGGCCTGGATGTGAATTCTAATTCTGAT
TCTTCCCTAGCCAAGTCTGGCACTTGGGTAAGTTACCTGTGTTGCCCAGGTTTCTCTGGGTGCTTCCAGCCTGAGTGCAAAGGAACAGGCCTCT
GTGCAGCAGGTCGCGCCCCTCTCTGTGTGTGCCCCTGGAGGATGTCTAATCTGTTCTAAGGGGACAGTTTGTCTTATGTATTGCAGCTGCCCGT
TCCCTGTGGCCATCTGCTGCAGCCAGAGCCACTGGGCCTATGTCATCAGCAGTGCCTGGCTGAGCAGAGCTTGGTCAGATCACTAGTGCTACAG
GTTCAAGGGCTCCAGCCTAGAGTCTGATTTAGGGTCTGGATGCTACCTGGTGCAACCTCATGTTTAACCAGCTCCTCTAATGCATCTGAAGCAG
GCATCTGTGGACTAGACAGCGAGAAGCTCTGCTCCAGCCTCGTGGTTCCCATCCTTGCCTGCACATTGTAATCCCCTGGAGAGCTTTAAAGATT
ACTGAGGCTTGGCCGGGTGCAGTGGCTCACGCCTGTAATCCCAGCACTTTGGGAGGCTGAGGCGGGTGGATCACCTGAGGTCGGGAGTTTGAGA
CCAGCCTGGCCTACATGGTGAAACCATGTCTCTACTAAAAATACAAAAAATTAGCCGGGTGTGGTGGTGGGCACCTGTAATCCCAGCTACTCGG
GAGGCTGAGGCAGGAGATGGCTTGACCCAGGAGGTGGAGGTTGCAGTGAGCTGAGATCACGCCACTGTGCTCCAGCCTAAGCTACAAGTGAGTG
AAACTCTCTCTCAAAGAAAAAAAAACTTACTGAGTCTTGAGTCTCACTCCCAGAGATTGTGGTGTAATTGGTCTGCGCTGTGGCATCCCTGTAG
AGATTTTTAAAAGGTCCCAGCTGGTCCTAGCGTATAGCCAGAGCTGAGAACCGCTGCCTGGAATACCTTCTCTCCCTAAGATGAGTTACTTCTA
GGATGCTCCTTGGTCTTTGGTATAGGTAGACCCAGAAGGCTGTTTGTGGCCCCTCCCACTAGTGCGATGACCCCCTTTCCCTCCCACGTGCAGT
CATTGTGAGCAGAGTCTAGGTTCAAGCTGTCCTTGCTTTATTGTCTTCTACAGCCTCATTTTTGTTTTCTGTGATAGAATGTTAGGACTTACCC
TTCTCAGGTTTCAGTGAGGTTACATGTGATAATGGTGTGTTACCTGTCTCAGGGCTTGGCCTGTGACAAAGGTGCTTAACAAGTCCTTGCTCTT
TTTTTCTTCTCCCCTTTCCTAATGGTTTCCAGTCAGTGACCAAGTGCTTTTTTTTTCTGTAAAGCAGTGAATTCGATTCTAGATAACATATATG
TGTTTCCTAGCCTCAAAGCCATTGATTAAGCTCTCGTTAAGGAGGCAGGACACTTAGGCCTAAAATCTAATAGTCTGTGGGGTTGAACTAAGGA
AGGGCAGAGCTGGGCTGGGCAGACAAGCTGTGTATGGTTTAGGAGTTGACGTATAGGAGGATCTGGTCTGGGGGGACCACATGAGCCTGCCACT
CAGGGAGGATCGCCTTATCTGTTATTACTAACAATGGGACCTTGCCAGTTTCCCGAGGAGGGATTTTGGTTTACATGTGATAGTCTTGCACTGA
ACCAATTATGCTGGTTACATGAGCCCTTTCCGTAGCCATTATTCCCTGACATTCCCCTGGGTGCTTGACTTCTGGCTCTAATATGCATTCGTCT
ACCAATTATGCTGGTTACATGAGCCCTTTCCGTAGCCATTATTCCCTGACATTCCCCTGGGTGCTTGAGTTCTGGCTCTAATATGCATTCGTCT
TTTGCCAGATCTGTGTCGAGGCCAATATTGCAAGTGGGAAGACGACATGCCTGGAATTCTTCTCCAACGCGACAGACGTCGAGGTACAGCCTCT
ATGCTAGGTTTGAAGGAAGCGGAGAAAGCTTGATGGGATAATTTATAGAGGGTGTGGACTAACCTTGTCCAATAGGACTATAATTTGAGCCACA
TTTGCCAGGCGGAACCAGGTGGATTCCTTGAGCCCACGAGTTCCAGACCAGCCTGGGCCACATGGCGAGACCCCATCTCTACAAAGATATGCCA
GGCATGGTGGTACGTGCCTGTAGCTCCAGCTACTTGGGAGGCTGAGGTGGGAGGATTGCTTAAGCCCAGGAGGTGGAGGTTGCAGTGAGCTGAG
ATCACGCCATTGCATTGCATCCTGAGCAGCAGGAGTGAAACCCTGTCTCAAAAAAAGAAAAAAACCCACAAAAAATAAAAAGGACATTTTATTT
CACATAACATATGCCAAATGTGATCATGTCAACATTTAATCAATATAAAAATCATCAATGAGCTATTTTGCATTCTATCTTGTTATACTAAGTC
TTTGGAATATGATACTGGTTGAGCATCCCTTATCTGAAATGCTTGAGACCAGAAGTGTTTTAGATTTCAGAATTTTTCAGATTTTGGAATATTT
GCATAAATGTAATGAGATATCTTGAGGATGGGACCCAAGCCAAAACACGAAATTCATTTATGTTTCATATATACATAGCCTGAAGGTAATTTTA
TACAATATTTTAAATAATTTTGTGAATGAAACAACTTTGTGTTAAGTATTTATGTGTGGAATTTTTCATTTGAAGTGTCATATCGGTTTCCAGA
AAGTTTTGGAGTTTGGAGCATTTACGATTTTGGATTTTTCAATTAGGGATGCTCAAACTGTTTGTATTTGTAAAATAAGTGTAACTTATAGCAT
ATCTTAATTTGGACTACTCATATTTCAAGGTCTTAATAGTTGCCTGTGGCTCATGGCCACTGTATTGGACAGTGCAAGTGTTGACTGCCTCAAA
GTCTGGTTAGGAAAAGAGTCTACATGGGTAAGAAGAAAGGACAGGCTATTGAGCAGGGCTAGAAGTGTGACATCTGAGATAGCAATGTCTGAGC
AGTGGGTATTACTATGCATAGGCAATTAATTTAATTTCTAGTTCATTTAAAGCCTAGTCACTAAAACTAACCTCAGAGATGTTTAAGAATCATC
TCTACACTAAGACATTGACCTGTCTTCCTTGTTCTAGATTTAGTGGTATAATCTTCTCTATTGATGGCTCTCTTTGTTACTACATAGAAGCTAC
AATTTCTATACTTTTATAGTCATTTTCTTTCCTTCTTCCCTTCCTTTCCTGTTTTTACTTCTGCTAGAAATATATGCATGGCCAGGTGTGGTGG
CTGATGCCTGTAATCCCAGCACTTTAGGAGGCTGAAGGGGGAGGATCACCTGAGCCCAGTAGTTTAAGAGTAGCCTGGCCAACATGACAAAACC
ACATGTCTACAAAAAATACAAAACTTAGCTGGATGTGGTGGCACATGCCTGTAGTCCCAGCTGCTCAAGAGGCTGAGGTGGGAGGATTATTTAA
GCCCAGGAGGTTGAGGCTGTGGTGAGCCAGGACTGTGCCGTTGAACTCCAACCTGGGTGACAGAGTGAGGCCCTGTCTCAAAAAAAAAAAAAAA
AAGAAAAAAGAAAGAAAGAAAAGAAATATATATAGTTTGATAAATAATAATAAACATTATATAGCCCTTGTCCTGAACTGTGTTCATGTGCTGC
TGTGCAGGAAAGAGTTAACATAGCAGACCTAAGACTGCTGCCCTTTAGAAATGCCTGCTTGCAAGGGTGGTCTTTGGCTGGCATCTGGGAACTT
AGATCTTGGGAGGGCCCCCACCATTCCTACTGATAACAGTGGCTCTCTGCTTATAAATTGTTTCTGCAACAGTATGGTTTATGCTGACACCTGC
TTTCTTCCTGGGAGTCCGGAATTTTGGTACATGTCAGGAAGTGGTTGTCTGCATGGCTGAGCGCTGATGAAAACCCTGAACTCCCAGGTTCAGG
CCAGCTTGCCTGCTAGACAACATTTCACATGTATTGTCACATCTTGTTGCCTGGGGPATTCAGTGTGTACTGTGAAGCTCCTCTATGAGAGGAC
CCTTGGAAGCTTGTCCCTGGTTTCCTCCAGACTTTGCACCTGCCCTTTTTCCCTTTGCTGATCCTTTTTTGTTTTTCTTCCTTTTTTTTTTTTT
TTTTTTTTTCTGAGATGGAGTCTTGCTCTGTCACCCAGGCTGGAGTGCAGTGGCATGATCTCGGCTCACTGCAACCTCCGCCTCCTGGGTTCAA
GCAATTCTCCTGCTTCAGCCTCCCAAGTAGCTGGGATTACAAGCACCCACCACCATGCCCAGCTAATTTTTTTGTATTTTTAGTAGAGATGGGG
TTTCACCATGTTGGCCAGGCTGGTTTCAAACTCCTGACCTCAAGTGATCCACCCACCTCAGCCTCCCAAAGTGCTGGGATTACAGGCGTGAGCC
ACCGTGCCCGGTCCCCTTTGCTGATCTTGTTTTGTGTCCTTTCGGTGTAATCCATCTTAGCTGTGAGTATGACTGCGTGTTGTGTCCTGTGAGT
TCTCATGAATCATCAAACCTGGGGAGGAGGGGGGGCTTGGGGACTCTTGACCCCTGTCCTGAGTGATATGACTCTTAATGGCACTTTAGAAGTG
CTTATAGTCTCACTGAAGACCTTGGACCCGATTAACAACGATAGTTAACATCCAGTGAGTATACTGCTATTTGCCCAACACTGTGTTGAGGGTT
CATCATAAATACTAAATGATCTCACCCTCACAAGAATAGTCCCTCTGAAGTAGGTACCATTTTTACCACAGTTTTAGATGAGGAAACCATGGCC
TAATGAGGTTAAGTGGCTTGTCCACGGTCGCTAGGCCAGTGTATGATACAGCGAGATCCCAGCCCCGATACAGCCAGATCCGAGCCCAGGCTGT
CTGGTTCCAGGGGCCACACCCTTGTTTGATTCTGTGCTACGAGAGCTTTGTTGCCTTTAGGATAGACCACTCTCCCTTAACATCTGAGCTCCGT
GTCCCCTGACCTTCCCAGCGAGGTTATGCTTCCTGACAGAGGATCCCCTGGCCGCTCTGTACCTCTCTTTTATACCACTTCTTGGGGTTTGAAA
ACATGTTGGCTTCTGTGATTACCTGAGTAATGTCTGCTTCCCCTGGTAGGCTTAAGGTTCTGTGAAGGCAGAGCCTGGGCTGGTTTTGCTATTG
TTGTGTCCTCAGTGCCTAGCATGGATATCTGGCACACAGTGGGCACTCAATAAATGTTTGTTGGGTGACAGTGCTATGGATGGAGCACCCCTGA
GCTGGGTCGGGGAGCAAGGCTGCCTGTGACCCAGACTCTGGTGGGCCTGTGTGGTCCTGTGTATGCTACTTAGGGGCTAGGAAGAACACCTAGA
AGAAGGAGAGGGGGTTTGAGAGGGGCAGGGGAAGGGCATATTTGAACTGGATTTTGAAGGGTATACAGGAGTTTTCCATGTGATGAAAGTTGAT
GTGAAGTGGAATGGGGTGGGAATGCTGGTCAGAAGAAACAACGTAAGCAAAGACACAGAGTATCTTGCTTGAGGGTGGATCACTTGAGGTCAGG
AGTGAAGGGTGGGGGGTGATTACTTTTCTTTTCTTTTTAGAGACAAAGTCTTGCTCTGTCACCCAGGCTGGAGTGCAGTAGCGCGATCTTGGCT
CACTGCAACCTTTGCCTCCTGGGTTCAAGTGATTCTCCTCCCTCAGCCTCCCGAGTATCTGGGATTACAGGCATGCACCACTACATCCGGCTAA
TTTTTGTATTTTTAGTAGAGATGGGGTTTCACCATGTTGGCCAGGCTGGACTTGAACTCCTGACCTCTAGTGATCTGCCTGCCTTGGCCTCCCA
AAGTGCTGGGATTACACCTGTGAGCCACTGCACCCAGCCAGGGTGGCTACTTTTCAAGAGTTCCATGCATTGCCTGCCATTGGTCTTTTGTCAC
CTATGGCCCCTTCTCACATGTACTCTGAAAGTTCCCAGATGGATCCTGGCACTCTGAAGATCTTGGCCCCAAGAACACCATTTAAATCTTTTCT
AGCACACACCTGCATACCCAGATAAGGCTCTAGCAGAGTCTGGGGTTGTCCCAGATTTGTCTGTCTACACCACAGATGGAGGCTCTAGAATGCC
CAGCAGAGATCCTGATATGTGTCTGAAAACACACAGGATGTTGGAGAGATTGGCAGTCATCCTAAAAGGTACAAACAGGACCAGAGGGAAAGAA
TGCCTCCAAACCAGTGAGACAGGGAGCGTACTAAAGAAACAGGGACTATCAGCAGCCAGTGCACCCAGTGCAGTGATCAGAGGCAGGAAACGGA
GGCAGCTGCCTGCCAAGGCCTAGCCCTGTCCAGTGTCCCTCCTGGCTTCTCCCACCCACCTGCCAGCTTGATGTCGTAGGCAGGCAATAGGTGG
GATAGAATACACAGAAGACGGATGGACAGAGCCACCTCTGACCTTGGGGAGGCTACCACGGCAGTAGAGAGGTAGGATGTACCTATGCTTGCCA
CATTGGGTAACATGGATAGAGTTATTCTCAGGACATTTCTGGAAGTAATGTGAGGAAGCCGTGTCTAGATAGAGTTGTGACCTCACGGGGGTTG
TCAGCTATTTTGGCCTACCTTGGGCACCCAAATTTGGATAGTCTTTTTCTCTCTTTTTCCCTTCTCTCCTCACTTTCCCCTCAACACTTACACA
CTTTTACAGGGTTCAGCACAGAGAAACAGTAGCTCTGATGTTTTCTTTCCTTCCAGGTGTTAACGGAGCCTGTGTCCAAGTGGAGAAATGTCCG
TGGCCACAATCCTCTGGTGAGTGGGTTGTTGGTTGCATGAACCCCCGGCTTCCCCTTAAGCACCTGAGAATGATGGTGCCTCTGCCTGAGCCCT
GCCTGCCCAGGGATTCTCTGCGCTCTTAACTGAGTTGGAGAAGACCAGGGAGGGACAAGCAGAAAATGGTGGCATGTTGGTGTTCTGAGGTCAA
GGATCTCTGAGGCCTGAGTGGCTGTGCACAGAGCCTAGCGACTTCCCGTCAGCTATCCCATACTATTCCCAGTGGCCGACCTTAGCAACTAGGG
GTGTCCATTTCTTGTTGCTCATTCCAGCCTTCCCCTCCCCTAGAAGAAACCTATCAGAACCCGGGACTTCTCAGGCCTAACCCCCCCAGGACCT
ATCTCTAAGTAACCAGGATCCTTGGTGGACCCCTGACTTGGGTTTGCAAACTTGGCCCTGTCTGGTAACAGGGGCCACCTCTGGCTTACCTGGA
AGCCACCACCCTGCTTTCCCAAGGGTGAACCTAGCAACAGGGGCTGTAGTGTGCTGTGTCCCTTCCTTCACCTTCCCCAATTCGAGGTTCTGCT
CTGGTTGCTTGAAAGAGATGGATAGAATCTGGGTCCTGCAAGAGAGAGCTTTAGAAGAGGATGTTGGTTCTGAGCTCCCATCCCATGAGGTAGG
GATGATTAGTCTCATTTTTCAGCTTAGGAAATTGCACTTATATAAGTTGCCTGAGGTCACACAGCTAGTAAATGGACTTGGAAGCCTGTGCTGC
CCTGTGATTTTTTTTTTTTTTTTTTGAGATGGAGTCTCAGTCTGTCCCTAGGCTGGAGTGCAGTGGCGCGATCTCGGCTCACTGCTACCTCTGT
CTCCCAGCTTCGCGATTCTCCTGCCTCACTCTCCTGACTAGCTCGGACTACAGGTGCGAAACGACCATGCCCAGCTAATTTTTGTGTTTTTAG
TAGAGACAGGGTTTTACCATGTTGGCCAGGATGGTCTCCATCTCCTGACCCTCATGAGCCACCAACCTCAGCCTCCCAATGTGCTGCCATTACG
GGCGTGAGCCACTGTGCCCGGCCATGCCCTCTGATTTTTAGCAACACAATTGGCATCTCCTGTGTGGTTCATTAGACTCTGAGTTCTACTAGGA
GCAGGGACTGTATCTACTTTGTTCATTGTTATTTGCTAAGAACCAAGGCACACTGCCTAGACTTAGTAGGAGCTTGTGCATTCATCGATTCATT
CAACCAATATTCAGTGCATGTCAGCCATGTGGCAGACACTGTTCTGGATGATATGGCAGTGATAAATTCAAAGCCCTTGCTCTCCTGCCCCTAA
TATTCCAGTGTGGGGAGGCAGAAGTCAAACATATAATCTGGCGGGTGGTGATAGGGCCTAGGAAGAAAACTAAAGCAGGTGAAAGGATGTTTCT
TAAATAAGGCAAAACCTCTCAGTCTTTCAATAAAGAATCCTAAAGAGACATGAGAAGAATTTCTTCTCTGTGCATTCGGGAGAAGATGGAATAC
CAGTCATCTTCTTTGGATATAACAAAGAAACCTCTGTAACATTCATATTCTCTAGAAGCCAAGGAGGAACCCAGGCTTAGTTATTAAGTAAGAC
AGCTAGAGTTGCAGGTGTTACGGATCATAAGTGACGTTTCACGGCACAGAATCACAGCCTCAGGAGTCTTAGTGTCCTTGGTACAGATAGCCTT
TTCAATGAGAGTTTCCTAATAAAAAATACAATAGAAGTAGCCATTTTTATATAAGTTTCCACTCTAGACCAAAAGAAAGATCGATACTTTTGTG
AAAAATCTCAATTCATCACTTTATCTGAATTGGTTTTCTGATGCAGAAGGTGACTGTTTCTGCGGCCATCAGGCAAAGAGGGAGTGTTTCTCCT
AGCCTTTATGAGATTTTCTCTCAACTGCAGTCAGAGGAGTCTGCAGTTCTCAATGAAGAACACTGGAGTTTCATTAGTTTGCACCAAGAGAAAA
ATGAAAAGCAAAAATGAGGAAAAGGCCGGGTGTGGTGGCTCATGCCTGTAATCCCAGCACTTTGGGAGGCCAAGATCAGAGGATCACTTAAAGC
CAGGAGTTTGAGACAACCCTGGTCAACATAATGAGACCTCCATCTCTAAAAGAAATAAAAGTAAATAAAATTAATTTAAAAATGAGGAAAGGAG
CAGAACAGGTTTCTTAGCAAGTATGCTGGAGCATGAAGTGTTTTGCTTCTAGA1&GGGATGGAGACTGATTGAGTAGGTCGGTTACTTCTGTTT
TACATGAGATAACCAGGGAATGTGTCTATGATAAGGTGCTTAAACATGAGGGTGTGAGCACTGCAGATATCTGGATGAAGAGTGTTTAGGCAGA
AGGCGCTGCAGAGGCAAAGGCCCTGAGATGGGACCATGCTTGGCATGTTTGAGGAATGCTAAGTAAGCTCATGTGGTTGGAAGCCAACCAGGCC
AGAGGGAGATGGGTAGGAGATGAGAGAAAGCCTTGTCGGCCATGATAAGGGTTTGGATTTCATTCTGCATGCAATGGGAAGCCACTGGAAGGTT
CTGAGCAGGGGAGTGGCAAGAACTGATTTAGGTTTCACAGACTCACTCTTGTGGCTGGATAGAGAACAGACTACAGAGGGCTCAAAGACCATTC
ACTGAACGACTTGAACGCATGTTCCTTGGTGATTTTATTTTATTTTTTGAATTTCCAAAGAAAAGTAATTTATTTTTTGCAGTTAGGGCAGCTG
AGAAGTCCAGGGTTGAAGGGGTGCATCTGCTGAGGGCTTTCTTGCTAGTGGGGACTCTTCAGAGTCCCCAGGTGGTACAGGGTGTCACATGGTG
AGGGAGTTGAGCATGCTAGGTCAGGTCTCTCTCCCTTTTCTCATAAAGCCACCAGTGTAAGATAACCCATTAATCTATTAATCCGTGAATAGAT
TAATCCATTCAAGAGGGCTCTGTACTCATGATCCAATCTTTTTTTTTTTTTTTTTTTTTGAGACAGAGTCTCACTCTGTCGCCCAGGCTGGAGT
GCACTGGCGCGATCTCGGCTCACTGCAAGCTCCGCCTCCTGGGTTCGCGCAATTCTCCTGCCTCAGCCTCCCGAGTAGCTGGGACTACAGGCGC
CCACCACCACACCTGCCTAATTTTTTGTATTTTTTAGTAGAGACAGGATTTCACCGTGTTAGCCAGGATGGTCTCCATCTCCTGACCTCGTGAT
CCGCGCACCTCGGCCTCCCAGAGTGCTGGGATTACAGACGTGAGCCACCATGCCCGGCCGATCCAGTCATTTCTTAAAGGCACCACCTGCTACA
TACTGCCACATGGGGATTAAGTTTTCCAACACATGAAATTTAGGGGATTCATTCAAACCAAAGTATTATTTGTTAAAGATGATCTTAAAGAGCT
TAACTTCTTTAAAAATPTTAAACTTATTTATTTATTTATTTATTTTGGAGATAGGGTCTTGCTATATCACCCAAQCTGGAGTGCACTGGCACAA
TTATAGCTCACTGTAACCTTTAACTCCTGGGCTCAAGTGATCCTCCTGCCTTGGGCTCCCAAAGTGCTGGGATTATAGGCATGAGCCACTGCAC
CTGGCTCTCTTTTCTTTATGCTACAAACATTCAAATTATTCTCTTATAGCTTTTTTTTTTTTTTTTTTTTTTTGACATGGAGTCTCGCTCTGTC
ACCCAGGCTGGAGTACAGTGGCATTATCTGCCTGGCCAGCATGGTGAAACCCCGTCTCTACTAAAAATACAAAAAAATTTAGCTGGGTGTGGTG
GTGCATGCCTGTGATCCTACCTACTTGGGAGGCTGAGGCAGGAGAATCGCTTGAACCCAGAAGGTAGAGGTTGCGGTGAGCTGAGATCATGCCA
CTGCACTTCAGTCTGGGTGACAGAGCAAGACTCTGTTTAAAAAAAAAAAAAAGATTTTTATCAAAAAGACAGGTGATAGGTGTGGAGAAAGGGG
ACCCAGGCTGGAGTACAGTGGCATTATCTGCCTGGCCAGCATGGTGAAACCCCGTCTCTACTAAAAATACAAAAAAATTTAGCTGGGTGTGGTG
TTTTTGCTCAGGATTGCTTTGGCTATTTGGGGTCTTTTGTGGTTTCATATCAATTTTAGGATTTTTTTTTTCCCTATTTTTGCAAAGACTGTCA
TAGGTATTTTGATAGAGATTACATTGAATCTGTAAATTGCTTTGGGTAGTATTGTCATTTTAATAGTATTAGTTCTTCTGATCCATGAGTGTTG
AATATCTCTTTTTTTGTGTGTGTTCTGTTCAATTTCTTTCATCAGTGTTTTATAGTTTTCCTTGTATAGATCTTTCACTTCTTTGGGTAAATAG
ATTCCTAGGTATTTTACTTTTATTAATTATTAATTAATTTATTTTTTTTTTCGAGACAGGGTCTAACTCTGTAAATCCAGGCTGGAGTACAGTG
GTACGATCTCGGCCCACTGCAGCCTCTGCCTCCTGGGTTCATACCGATTCTCATGTCTCAGCCTCTAGAGTAGCTACGACACAGGTGCGTCCCA
CCACACCCAGCTAATTTTTTGTATTTTTAATAGAGACAGGATTTCGTCATGTTGGCCAGGCTGGTCTTGAACTCCTGGCCTTGAGTGATCTGCC
TGCCTCAACCTCCCAAAGTGCTGGGATTACAGGCATGAGCCACTGTGCCTGGCTGGTATTTTATATTCTTTGTAGCATCGCAAATGGGATTCCT
TTCTTGATTTCTTTCTCAGGTTGTTCACTGTTGGTGTATATAAATGCTACTGATTTTTATATGTTGATTTGTATCCTGTAATATTACTGAGTTC
CTTCATCAGTTCTAACAGTTGTTTGGTGGAGTCTTTAACGTTTTTTTTTTTTTTTTTTGGTCATTTCAACTTTTATTTTACTTTTAGCGGGGTA
CATGTGCAGGTTTGTTACCTGGCTATATTTCATGATGCTGAGGTTTAGGGTATGATGGATCCCATCACCTAGGTACTGAGCATAGTACCCAATA
GGTACTTTTTCAACCTTTCCCCCCTCCCTTCCTCCCCACTCTAGGAGTCCCCAGTGTCTATTGTTGCCATCTTTATCTCCATCAGTACCCATTG
TACCCACTTATAAATATCCACTTAAGTGAGAACATGCAGTATTTGCTTTTCTCTTCCTCCATTAAATTAGCTTAGGATAATGGCCTGACTCTTT
AAGCTTTTCTAAGTGTAAGATCATGTTGTCCGTGAACAAGGTTAAGATGACTTCTTCCTTTCCAATTTGGATGCCCTTCATTTCTTTGTCTGGC
CTAATTCCTCCGGTCCCTCGGACACTTTCCATAGGCTCCTATTATAGCTGTTACCATACTGTCCTATAATTGTTTCAGTGTCTGCCTTTGTCCA
CCCTGCTGTCAGAGCCCTGTGATCATGCTCGGTTTATCTCCAGCCCCATCCCTGCCTGTGTAGGTTTCTTGAGCTGTCCTTCAGTGCCTTGTGA
GACCAGGGTCCTCCTGCAGATGCCACTTTGATCGAAAGTGCTGTGTTTTCCCTTCTGCAGGGCCTGATGTACCACGATGCCTCTCGCTGGGGTC
TTACGCTACAGACTTATGTGCAGCTCACCATGCTCGACAQGCATACTCGTCCTCAGCTAGGTTTCAGATGCTCAGTTTCAGCCTTCTTAGAAGT
TTTCTTCAGACTTGGGCTATGTGATTGCCAGGAAGGGAAACTTTACAAGGAAGCCAGGCTAGGCCTTCAGTGCTGTGCAGGCCTCTCTGTGTGG
GGCCAAGGTCTCTGGCTTCTGTGGTTCTCTTCTGACCTGCCTCTAGTGCCCCCACCTGCAAACCTCAGCCAGCCAGGTGGTGCCCCTGCCACCA
TTCTGAGCTCACATCTTTATGACCTGACCACTGCTGCGCTTAATGCCGTTTATACTCAGTTCTTCTCAAGTGTGCTCCCTGGACCAGCAGCGGA
AGCATCACCTGGAAACTTGTTAGAATGCACATTTTCAGCTGGGTGTGGTGGATCCCACCTGTAATCCCAGAAACTTTGGGAGGCTGAGGCATGC
GGATCACCTGAGGTCAGGAGTTCAAGACCAGCCTGGCCAACATGGTGAAACCCCATCTCTACTAAAAAATACAAAAATTAGTCTGGCGTGGTGG
TATGTGTGTGCCTATAATCCCAGCCACTTGGGAGGCTGAGGCAGGAGAATGGCTTGAACCCAGGAGGCGGAGGTTGCAGTGAGCTGAGATCATA
CCACTGCACTCCAGCCTGGGTGACAGAGTGAGACTCTGTCTCAAAAAAAAAAAAAAAGAAAGAAATGCAAATTTTCTAGTCACATCCCAGACCT
GCTGAATTAGAAATTCTAGGGATGAGGCCCAGCAATCTGTGTTTTATCAAGCCCTCCAGGTGACTCGAATGCATGCTTTGTTTGTTTTCATATT
TGAGAACCACTTATTTAACCTTCATGGGGTCTGGCTCTCTAGTTCAAATTCTTGAGACAAAGAATCTGATTGGTTCAGCTTTGGTCAGTGTCCA
GCCCTGCTTCAATCAGGTGTTGGGGACAGGACCCCAAGGCACAAACATGGCTGCTTAGGCCTATTCGATCAGTGAGGATGCATCTAGAGACAGA
GTCTCTTAGAAGAAAGGGGGCTGTGGAGGTCCTGATCAGCACCTATGTTTCAAAGCTGTAAGCACAACCTATGATGCCAGCTTTAAGGCTCTGC
ATGAAGGAGACAGACTGTCCCTACAGCCAGTTAACACTGGCACAGCTGCTGGGCTGGACAGTTCGGTCCCAGTCTGGGTACCTGCAGCAATAGC
TACCATTCTTCTGTAAGTCTCTCTTTTAAAATTGAAAGATGTGAGTGGGCCGTTTTTGGTGAATAAGAATTTCCATCTCCCCAAATATCTGTCT
AAATTGCACAAATCCACAAATCGAAAGATCTGAGGTTAGTGCATTTTGCCTTTGCTAATTTGCTTTTCCTGAGCAGGCACTCAATTGCAAACCT
AAGTTGTATCTAATGATATTTGAATAGGAAGGGATATAAATGCACTCCTCTATTTAAGGAAATCTACCTCAGGAGGCAGAGAGAGATGGACAGA
CCAGGAAAGATGAGCAGAAGGAGACCAGAGGGATGAATGGGGAGCTAGGAAGTAAGGAAAGAGAGGATGGAAGAATGGAAAGGAAGATGGATGG
GCAGAGGATGGATGGACAGAGGGACAGCAGGGAGAGGCAGGAAGTGCCTGGGTTATGGCTTTGAATCTGCCTTGGATTTGTTGGCCCAGGTCCC
ATGGGTGCTGTGGGCCATGGCCAAGTGAGAGTAGTGTTAGTGTGCCTGGCATCAAGGGTCAGGGCGCCTCTTGCTTCCCAGAGGAACGCTGGAC
AAAGGGCAAGGTCGTCTCCAGAGGGTTTGAGCATCCTGGCAGAAAGGGGCACTGTCAGGCTGTCTTCCTCCCTCTTGGGCATCGTTCTTACCAG
TTTCCTTGGGCAGAGCCCCCAGAACGTCACCCCCAACCCGACCCTCTTGGGGCTGTACCTAATGCATGTGGTCCCTCTGCTTAAGCAAAGTCAC
CCTATTTAGGTCCCTGGTAGTCACCACATGTTGCCTGAAGTCGACGCTTTGGATGGTTTCTCCAGGGCTTGACCGTGTTCTAGTTGAGACTGAA
CCTGCCATCAAAGAGGCAGCTGCTGATGAGCCCTCAGCCTCGGCATTGCTGGGCCATGCATGAAGCTTTGGCGTGTGGGGCACGTCAGGGTGGA
AGGGGGACACAGCCTGAGCCTGGTGGCACATGTGGCTTCTCCTCCCGGCAGGGGAGTGAGGAGGTCCTCCTGCTTCACTCTCTCTGCCGCCTTG
ATTCTCATATTCAGCAAAGTCCCCAGGCAGGGGGAGTAACAGACATTTCTCAAGGCCTCCTGCCTGTCTCCTTTTCCCCTGAGTTAGTGATTCT
CTTTTTGTTTTCCTTTAGGTGTCATCTGTACGGTTGATGGAGAGGTCGATTCACAGCGCAAGATACATTTTTGTAGAAAACCTGTATAGAAGGT
ACTGTAGTTTGAATAGTTGATAATTTATTAAACCACTAATTTTTCAAAATGAAATAGAAACTTGTTTATTCGATTGACAGATATGGAGTACCCG
CTGTTGTATGAGTTATCCATTGCCATAGGAATGCGGTGTAACAAACAGCCACGAATCCTCATGGCAGACAGCTATCTGCATTCATTGCCCAGTC
AGCCAGGGTGGTCACCAGGCAGCTCTGCTTACCTTGGCTAGGCTCAATGAGATGTCTGGGGATCAGCCGATGTAGGATAGCCTTAGCAAGGGTG
ACTGGAGGTGGCTCATCTCTTTTCCTCTTGCCTGTCACTCTCCTTCTCGGACCAGCACGAGCCCACGTGTCCACTTCTCACGGTGACAAAAAGG
GTAGAAGCAACAGCAGAGATGCAAGGACATATTTTTCACGCCTCTGCTCCAAGTTTGCTAGCATTCCATTGGCCAAAGCAGATCACATGGTCAC
ATGAGCCCAGGGTGGAAGGTGGGAGGGTCCAGTGAGTTACACAGCATGGTACATAGGCTAGGGAAAGAGGTGGAGAGTTGGGCGAGTCAGACGC
ACAGTGTGCCTCGCATTTGTTGTGTTTTTCACTAAAAGCTTCCCTCATGAGAGGAGACTCTGTGATGTATACCTAGTTAGGGGCTTCTGCTGCC
CTGTCAGTGCCAAGGGCTCAGGCATGGGGCCCTCCATCCCTGTGCTCAGAGACTGCCGATTTCAAGGGGTCTTGGAAATGGGTTGCAGACCCCA
CCTGTCCCTGCCGCGAGCTTGGCATACTGCTGTCCTTCCCTCGCAGAGCTGTTCGGATCTCTACGCTAGAGGCCCTTCCTCCCACTCTCATCTG
CAGCTGTCTTCCTAATGCCACCCTGTCTCCCAGCCCTCCCCGACCACGCTGTCTAGATAGGGCAAAGCTCCTTGTTAGTCTTTGTTTCGTTTTT
GGTTTTGGTTTTTGTTTTTTGAGGCAGGGTCTGGCTCTATCACCCAGCTGGAGTACGGGGGGAACGATCTCAGCTCACTGCAACCTCCGTCTCT
CAGGCTCAAGCCATCGTCCTGCCTCAGCCTCCCGAGTAGCTGGGACTACAAGAGAACACCACCACACCTCGCTATTTTTTTTGTATTTTTAGTA
GAGGCAGGATTTCGTCATATTGCCCAGGTTGGTCTTGAACTCCTGGGCTCAAGTGATCCTCCTGCCTTCGCCTCCCATAGTGCTGGGATTATAG
GTGTGAGCCACTGTGCCTCGTCCCCTTGTTATTCTTAAGGACTACTTTCTGTTAGTTTTCTTTGTAGTATTTAAGGCAATCATATGAAATTGTT
TAACCAATTATGAGCTACAAAAATGGCAGTTTTGTGTGGTTCAACTTAGTATGTGTCTAATTGTTTATTGCCACCCACCCCCCCGGACCATAAG
CTCCTAAGGGCAGGCACCAAGTTTGTTGTCCTTGCCTAGGGTGGACCCAGCACAGAGCACGCTCCTCGTTGATACCTGTTAGCTATTGTTGGAT
GAGGGGACCTGTGGCTGTGCTTTGGTCTCCAGCCCAACTTGTTCCCCAGGCTCTGGTCCCCCACTGTTTTGAACCTTTCATTTGAGGGGCCACT
AAAAAGGCTTCTCCTAGGAGCCCTGAGCTGCCATGGATCATGCCTTTGAGCCTGTGTCACCCCCCAAAATGAGAGCCTCTGCCCCAAATCCACC
CAGAGCATCAGAGCAATGCTGTGGGCTCAGGGGCTGGCAGCCTAGGTGAGTCCTGGCCGTGGCTTTGCCCCCTGGCCTCAGACTTTTCTTGTGG
GCTGCAGCTTCCCCTCTGACCAGGTGGCATTCCTGACCTTACGGCCCCATTTTCACCTGACAACCTGGGTTTCCAAATGTCCACTGCTAGGTTT
ATCCAGCCCTCTGCGGTCCTCAGCTCCTGCTCCTTAGCACCTCAGCCAGAGAAAGGACAGGCAGGAGAGAGGAGAGCGAGAGCAGAAGGGAGCC
TCTGTTATGTTTGTGTACTGCTCTGATTAGAACTGTAACCCGAGGCGGAGGTTGCAATGAGCCGAGATTGCGCCACTGCACTCCAGCCTGGGCA
ACAGAGCGAGACTCCGTCTCAAAAAAAGAACTGTAGCCTGAGGAACAGATCTTGGCAGTGGGGTGGGATTACCTATGAACCTCGCAGAGTGGGG
CTGTGTCTGTCTTTTAATCACATTCAGCTTGGCCTGTTCTGTGTGGGGTCAGTGGCTCTTGTTGCTTTTTTTATTTTTGAGATGGGCTTGTGCT
CTGTCACCCAGACTGTAGTGCAGTGCCATGATCATGGCTCACTGCGGCCTCGATGTCCTGGGCTCAGAGATCCTCCTACCTCAGCCTCCTGTAA
TAGCTGGACCTACAGCACATGTCACCACTTCTGGCTAATTTATTTCTTATTTTTTGTACAGACAGGGTCTCCCTGTGTTCCCCAGGCTGGTCTC
AAACTCCTGGGCTCAAGCGATCCTCCTGCCTCAGCTTCCCAAAGTACTGGGATTAAAGGCATGAGCCTGATTACAGCTCTGCTCGGCCTCGTGG
CTCTTTTTCAAGCAAGTAATCTGGATCGAATGACCAGAGGTTTAGGGGGAGAGGATAGAGTGTGGCATTTGCTGGGCCTGGGTTCTATCCTGGC
TCTGAAGTTTGCTCTGTTTGTGACCAAGACCAAGTTTCTAATTTCTCTGAGCCCATCTCTGATCTCTCATTCCTCTTGTCCATCTCTGAAAGAG
CCTTTGGACCTGCCACCTCCTTCGTGTCTTAGGACCCTTTTGTGGGTCCTTGCCACCTTGGTCTTTATTTAAATTTGTGAGTTCCCCAATCCTA
CCCCCAAACCTGTAGGCCACCCAGCGCTAGGTAGATTTCCCCAGGAGCCATTTGTCTTTGTTTCGCCGGTCACACTTCCTGCTGATGGCCATGG
GCTTTGGTAACTCACGCCCATGGTCCTGCATCACCACATTTCTGATGCGTCAGTTCTGTATCTCCCAATCTCTGTGGTTACGGCATTGGTCTAG
ATGACATGGGCCTTGCCTGGAGCTGTTCTGTGGGTAGTTTAGGAGAGTATATGTAGCTTTTATTTTTTTAGCTCTTTTCCTGTCCTGATGACTT
TATATTTATGTCTCTCCCCTGGCCCTCCCAGAATGCATGGCCTCTGTGGTTAGGATTTCTGTGGGACAATGAACCACAGAAGAAACTTACATGT
CCTTTCTGTTCTTGGGATCTAATTGTTTTAGTTCATATGGCTTTCGGGGGCTCAGATTCCCCTGAAAAGACCTTGTTGGAACCTTGAGATCCAT
CACTGGGATCTCTTGAAGCCAAAGGCTCTCAGTGGGCATGTGGCCTCGGGCACTCCAGAGGCTACTGGGAGCACTGTGGCTGCCTGGAGGGCCA
GGCTATCTACCATGGTCTCCAGAGAGATGAGAAAAGGCGGTGTTCCTCAGACCCTTTCATCGGATTCCTCTGAAGGCAGAGGAAAGCTCCCCAC
AGCACTGCTTCCTCATCCTCCCAGACCCTGTGGTCCAAAGCAGCCACCTGCCAACAACCCAGAAGTTTCTTGGAGTCTCCTATTTTAACCTAGA
ATTCATTGGGATTTTGCATTTTCCTCCATAATATGTTCTTAAGTTTGTATCTAAAAATAATAATTTACATAACTTATCATTGAGGACAATGGAT
GTTCCTGTATTAAACACCATGCTTTGCATGTGGCTTGGAGTTCCCACTCCCTCAGCCACATACCCCAGAGTCCCTAAGTCCAGGAGGCAGAGAA
CCAAGGGGTAGCGGCATTCAAAACACCTTCCCAGGGCAAGTGATTCACTTGTGAACTGCACCTGCTTATGTGTTCCCTGGAAACGGGGGCATCT
GCTCTCATGGTTAAGGAACAGGGCAGGGACCCATGGACAGGAGCTGCATTTACTGTCTGGTTGTTGACCCTATTGGGCAGATAAGGAACTGAGA
AAGGATTACAAAGCCAGTGCTCCTTCCTGATCGGGCAGGAACTAACCACAGGAGGGGAAGGTGTTCCGAACCCTGCTGTGTGGGGGCAAGCAGC
AAATGAGGAAGTGGCAGGAGGCATCTAGATTTCTCTCATCAAGGCCAAAGGCTTCAGGGCCCACTGGGTTCCCCAAAGCTGAGAATCCTGAGGT
ACTAGCCCAAGGGGGAGACTCTGCCTCTTTCCAGACAGGCTTTACCCTAAGCTTCTCCCACAACCACCTGTGCATCTTGTTTACAGGCAGATTC
CACTTCAGTAGCACTGAGGTGGGGCCTGAGAGGCCACATTTCTAGCAGGTGCCCAGGTAAGACCATACTCCTACTGGTCGGGGATCAAACTTTG
AGGAATACGGGCCTCAGAAGAAGGTGGCAGGGCCCGGGTGTAGTGGCTTATACTTGTAATCCCAGCACTTTGGGAGAAGTTTGGGAAGGATCAC
TTGAAGCCAGGAGTTCGAGACCAGCCTGGGCAACAAAGTAAGACTCCCATCTCTACAAAAAATTTAAAAAATTAGCTGGGCCTGGTGGTGCACA
TCTGTAGTCCCAGCTACTTAGGAGGCTGAGGCAGGAGGATTACTTGAGCCCAGGAGGTCAGGGCTGCAGTGAGCTATGAGCATACCACTGCACT
CCAGCCCAAGTGACACAGTGGGACTTGTCTCAAAAAATAAAAATAAAAAAAGCTGGTAGCCAGGTGGTCCACAGGGCGCCCTCCATCCCAATTG
CCACGACCCTTTTCCCCTGGACTTCTGACGGTAGGTGGGCTTCCTTGGGGCTTCACTGGACCTTTGGGGGTCATAAGTACTAAGAGCCTCGGCC
TGAGGGTTGTCTTTGGGACCATGGTGAAAAGTTCTGCGCAGTGCAGCATTTCCTGTTTCCTGCACCTGGCCTGTGGTTTCTTAATTTCACATTT
GTTGCTTCATATTTGGGTTGCTGGGGCTGTGTTGTGTTATCTTTGTTTTTAAGGTTTCTGGACTTATTCCTAGGCCAAGAATCAGGCTAGCCAT
AGGGCAAAGTAAAAAGTATTTTTAGCCAGAGCCACAGAAGGGATAAAGGGAAGGAAGACTAGAGCTTGGCCACTCTCCGGACAACTCCAGCTGC
CCTCGATGGAGCGGTCATCCACAACAAAGTATTACTGAGAAAGAACATGTGGGGTTTTTTTGTTTTTTGTTTTTTGTTTTTTTGATTTTGAGAC
AGAATCTTGCTCTGTTGCTCAGGCTGCATCACTCAGGCGATCTCAGCTACTGCATACCTCTGCTTCCCAGATTCAAGCAATTCTCCTGCCTCAG
CCTCCCGAGTAGCTGGGATTACACGCGCCCACCACCACGCCTGGCTCATTTTTGTATTTTTAGTAGAGACAGGGTTTTACCATGTTGGCCAGGC
CGGTCTCGAACTCCTGACCTCAAGTGATCTGCCTGCCTCAGCCTCCCAAAGTGTTGGGATTATGAGCGTGAGCCACCATGCCTGGCCAAAGTAG
ATGTTTTTAGAGGTTTCGGCTCCTTCAAAATGCAGATTACTGTCATTGTCACTACCTATGCAAAATGTAGCATAGAACTGACTTTGGCTGGGTC
CTTTGCCCATACCTAGAATAAGGTGTCTTGGTTCCCACCTGCAGGGAGTTCAGCCAGGTACTGGGAACCACACGGTGGCATGTGGGGCGTCAGC
TCCTGCCTGGGAGCTCGTGTTTCCTCATTTCCTGGGATGCTCTAGGAAAAGGCTTCCCAAAATGCCATGATGGGGAGCCCTTCACAAGGTGGGA
GGGCACATTACCATTGCCCCTTCAGTGCTTGGGTTGATCTTTTTCTCCCTCTCAGAAGCTCTTGCAACCTTGGTTCAGGATCTGTGGGGGCCTA
GACAGAAGGCTATATGGCAGGCTCAGGTCCCCAGAAAAGCCCACATATTGGAATCCTGCATCTCCTCGAAGTAAAATTTCTTGAAGGGTGTTCT
TGCTCTTTGGGCTGTGAACAAATGAAGAGCTCGCCACGGAAAAGACCCACCTCTTCTGGCACTGTCTACCCTGGCGAGGTTGGGGTGTGCAGAA
TGTAACTGCACTTATGCCACCAGGGATTCGAGAGGGCTGTGTTAATTGCTGGAGAGGAAGATTCCAGTTACACAGTATACCTTAAGACGGGAAC
TTCTTTTTCTTTTTTTTGAGACGGAGTTTTGCTGTTATTGCCCAGGCTGGAGTGCAGTGGTGCAATCTCGGCTCACTGCAGCCTCCACCTCCCG
GGTTCAAGCGATTCTCCTGCCTCAGCCTCCCGAGTAGCTGGGATTAGTCATGTGCCACAACGCCCGGCTAATTTTGTATTTTTAGTATAGATGG
GGTTTCTCCATGGTGGTCAGGCTAGTCTCGAACTCCCGACCTCAGGTGATCTGCCTGCCTCAGCCTGCCAAAGTGCTGGGATTACAGGCGTTAG
CCATTGCGTCTGGCCAAAGACAGCAACGTGTAAGGGGCAGCAGCTGGCTGGCATGAAAAGCTGAAGGTGGTGAGCAAGATTCATGTAGCTAAAA
GAGGCCACACAGGTGACTGTGTGCGCCGAGTAATGGGAAGCAAAGGTTTTACAAGATTGCCTGGTCGTCCTTTTCTAAGAACAGAAGTGAGGGC
GTGGAGTGGGACCCAGCTCGGGAAGTGGCATGCTGACTCACCTGTGGCTTGTGGTTGGGGACACTGAGTAAGACCTGGTGTTTGGAACCACAGG
CTGGGCCTCCTCCCCTGCTGATTGGCCCAGGGGCTTAAGTTCTTCCCAGCTCCTGTCTAGACTGAAACCTCCTGGTGTGGGTCATGCGCCTAAG
AGCTGTGGCCTTCCTGGAGCTCAGGCTAGAGAGACACTGGACTTAATCCGAGTCAGCTGACAGACTGGCGTGGGCGGGTGGTGCAATGGAGAGC
ATTTGGGTTTGGGGGTAGGGAGAGCCAGGCTTGGACCCCTGCTCAGACAGAGTTGGCCTTCACTTCTCTGATCCTAAAGGTTTCTTCTATAACT
GGGACTGAAGCCAGCCTCACAAGGTCTTGTGAAAACAAAAGGAAATAACGTATACAAGGGAGCTCTTCCATTCCATTCCGCACAGACTGGGGGG
CTCTCTTGGCCTCAGGGAAGGATGCAAGAAATTGTACTGATGTAACAGCAGGATTCATGTGGATCAAGACCTTGGTTTTGCCCCCTTGGGGGTA
GGGCAAGGGTGAGACCCCCATGCCCACTGTTGAGTTGTGTGCTTGGATTTGCCTAGCTTGGATTTGCTTGGTATTGCTCTCCGTGCATTCTGTA
GAAATAAAAGGCAGTGGCTTGTCTTGTCGGTGGTGTGGGGAGCCCCATGCTTGAGACAGACACAGGAGGAGCTGGGCAGAGTTCCTAGGAGAAA
TACAGTTAGAGCAGCGCCCACTCTGCCCCATGTCCCTCGGAGTCATCGAGATCACGCTTCTGTTTCCTCACTCAGCTCAGACACTGGTCATTGA
GTCCTCCTGGATCCCCCAGCCTCTTCTGGCACATGGTTCTACTCATGGAAGGGTTTTCAATAGGTCTGGCCTACTCACCCTGCCCATGGGAGGC
CACCCGACCAAGGACCCCTTCCAAGAGCATTGGGAAGGGAGAGGCTGCCCCTCAGTCTGCACCAACCCTGTGGGGGAAGCCTTTTGTTTGGCTG
TGATGGGGGTGGGAGTGGGAGGCCCCTGTGACTACCTGGAGCCGATTCCCAGGGGAGCTGTGGCAGGGGCAGAGCCACCGGAGTCTGTCAGTCC
CAGAGTCCCCCACAGGTCTGACCCCCTCCCCATCTCTCCATGTACAGACCCCCGTGTACTGTGACTGTTTTGCAGTGGTGGTTGGTTTGGTTTT
AGTTAACACTGCTTCATTTGTTCTGAGCTGTGGTTCAGTTCCATGATCATATCTGTGGGCACCCTCCAAGCTCTGGGATTGGACAGAGTCTCCC
TCGAGCTGTGCAGACGCATGAGGTACTCCGGTGGCTAGTCCAGCCTTGGACACTTGGGAGACTGTCCCATGGGCAGCTCAGCTTCTCCCCAGTT
GCTGCTAACTGGCCCCACCCCTTTGGTGCCACCTCTTCTGTGCCCTCAAGCCATGAGTTTGCTGGAGTAGGCGAGAAGCTAATCAGCAGCTTCC
TCATTGCACATCCTCATTTTCTTAGAGTCAATTTTGTTCTCAGTCTTCTTTTTCCAGAAAAGGAAAATAAGAGATAGTGGTTTGGTTTCTTGAA
CATCAAATATTCGGAGCTGAACTAACTGAAACCCAACTCAGGGTAGCCTGAACAATGAGGTGTCATCAACTCCCAACAGATGGCTTATTTGGTG
CCTCTGGCCTGGTTTCTCTACATTTTGCTACTGTGCATCCTGTTCGTGTCCTCATGCAGTTCTCTCAGGGTGACAATGTAGTCGTGGCAGCATT
GGGCTTCTCACTCGCTGGTTGGAGTAAGGGCAGGTGTCTTGCTAGTGGCTCTTCCCTAAGCGTGCGGAAGTATCTTTTCTGTAAGTCCCTGCAA
ACCTCCCTCACGCCTGCCTAMCCAAATTGGGCCCCTCGCTGGGGGAGGCCCTGCACTGATTCACTTAGCTTGGCTTCCTGAACGCATTGACTGG
AACTAGGGGAGACGACTTCCTTTAGACCGATTGTGCCTACCCTAAAGCTTGGAAGCTTGGGGTGGGGTGAGGTTTCCCTGAAGCCCTTGGGTCT
GTGGATGGACCCTGAATAAAGAATGGGCTACTTTTAAGGAGGTAAGCGGAGAGGAGGCAGGGAGGCAGCCTGTGGACACAAAGTTGTCACCTGA
ACTTGTAGTGCCTGAGTTTTGTTGCCTGGGAGTCCCCTCTGCAATGCTGTAGGATTTCCTTCCCACTCCTCCAGCCTCCCACCCAGCTTCTCCA
GGTTTTCTGGGAGTCCTGGCCGTCCCACAGGTTGGTCTGCCCTGGCCTCTGTGCTGCAGTCTGCTCAGGCCCCGCTGCTGTTCTCCGTCCACTC
TCCCGCTCCTCTGTCAATACTCTTCTCTTCTGAGGACATGGGGGTGGTCTCAGAGACTAGGGGAGAAATGTCCCTCTCCAGTTGGTGCCATGCC
TTGCTGTTAGGGCCCCAAGACAGGGCTACTTGTGCCCTTGGCCTCCCAGAGTGCCTGCCTCACTCTTCCAGCCTCTTACTTCCTTCCATGCTCA
TCCCCTCGTGGGACTGCAGTCAGCCCCTCCCTGCCCCCTCCCACTGAGAGCCTCCCATTGCACATGGCTCAAAATACGGCAACTCCCCCATGGC
CAGTCTTGCCGAACCCAGCTCTGAGCGGGACCCACATGGAGCCACTAGTGGAATGGAGGATGTTAAAGGGAGAATTCCTCTGCTAGGCCTGTGA
GGGTTGTCTGCCTGCTGATGGGCTTGTGCCCTCCAGCTGTGTCCATTCCTGCTCTCTCTTTCCTGTTCATTTCCTCCAAGGGCACAGGAAACCC
ACAGGAATACCAGGAATAGATGCCAGATTTCTCCTTCACCCTGGCTCGTTCTTCCTTCCAAACTTTCTTGCCTTTTTTTCTTTCTTTTAAGTTA
ACATTTAAAATGTGTAGTAGGCCGGGCGCAGTGGCTCATGCCTGTAATCCCAGCACTTTGGGAGGCCGAGGCGGGTGGATCATTTGAGGTCAGG
AGTTCGAGACCAGCCTGGCCAACATAGTGAAACCCCGTGTCTACTAAAAATACAAAAATTAGCCAGGCATGGTGGCGCACACCTGTAGTCCCAG
CTACTCAGGAGGCTGAGGCAGGAGAATCGCTTGAACCTAGGAGGCGGAGATTGCTGTGAGCTGAGATCACGCCACTGCACTCCAGCCTGGGCGA
CAGAGTGAAACTCTGTCTTAAAAAAATAAAAAATAGACCATCCTGGCTAACACGGTGAAACCCTGTCTCTACTAAAAATACAAAAAATTAGCCC
GGCGTGGTGGCGGGTGCCTGTAGTCCCAGCTACTCGGGAGGCTGAGGCAGGAGAATGGCGTGAACCTGGGGGGCGGAGCTTGCAGTGAGCCAAG
ATCACACTACTGCACTCCAGCCTGGGCGACAGAGCGACACTACGTCTCAAAAAATAAAAAAAAAAAATAAATAAAAACTAAAATATGTAGTAAT
GCTCCCCAACTCCCACTGCTGATTTGAGAAGTAATGTGTTGTTAAAATTTGGAAAATACAAAAAGGTATAAAGAAAAAAAGAAAAGTCTCATGT
AGTCCTGCAGCTTGAGAAAAGCACTTTTTACATTTTGATATTTTTCTTTCCCACCTTTCCAAGATGCATTGGTGAATACTTTTGGAAATGTGGT
TGGGATATACACAGTTTTAGATCCTGTTGCTTTCACTGAACATTAGATTTGAAATTTTTCCATGCCATTAAATATTATTTTACAATATCATCCT
TCAGTTACTGCGTGAATTTTCTGTTATATAGATTTACCAAGATTTAGTTTTTTATTGGTTTAAACAGTGCCGCGGTGAGCATCATGATTATTTT
CACAAAATAAATTTTTTTGTTTTGTTTGAGTCAGGGGTCTCACTCTGTTGCCCAGGCTGGAGTGCAGTGGCATGATCATGACCCACTACAGCCT
TAAACCCCTCGGCTCAAGTGATCCACCTGCCTCAGTTTCCCGAGTAGCTAGGACCATAGGCATGCGCCACCACCCCTAGTTAATTTTTTTGTAG
TTTTTGTAGAGATGGGGGTCTTTCCATGTTGCCCAGGCAGATCTTGAACTTCTGGCCTCAAGTGATCCTCCCACCTCACCCTCCCAAAGTGCTG
GGATTACAGGCATAAGCCACCACCCTGGTCCACAAAATAAATTTCTAGAAGTGAAATTGGTTTGCTGATTCATTGCCTAACCTGAGCACTGTCT
CTGTTCTCAGGGTACAAGCTTTAAAGTCCCTTTCTCTGCTTAGAGTTCCAATCCCAAGTGAATTCTCCCATCACTCCGTGCCATGGCCCTTGCA
GCATTCTGCCATAAGCCGCTCCCGGCAGCACGAGGTCGGGAATGCTTAGGGTCCTTGTAAACTGCATGTCCAGTATTGCACGGATCACACTGTG
CCCTGGTACCCTTGGTGACTCCTTTCATTGAGAGCCGTCTCAAGATCCTTTTGGGGGAATTGCATAGCCCAGAAGTTTTCTCTTAGGTGAATAA
GCCCTTACAACTCTCATTGCAGTGGGAAGATGCCAGAAGTGGACTATGTAGTTCTGTCGGAATGGTTTGACTGGATCTTGAGGAACATGGACGT
GTCTGTTGATTTGATAGGTGAGATGCACCCCTCCCACCTCCCTCTCTTGTCTCTCTCCTGGGTCCCTTGAGGATGTGCTTGGCTGAGCCCCAAC
CCTTGGGGGCAGTGGGTGAGGCACTTGCCCTTGGCAGTTCCTTCCCCATGGGAGTTCAGCTTTCTCCCTGAGGGAAAGAAAAATTCGGGACTTC
TGGTCTCTCATGGAGAAGAGAACCTGGCCTTTGTTCTTTAAACTTCCCTCCCTGGCTCAGCTACTTTCAGACCCAATCCACTGTGATCTGATGG
GTTTTTCTAGTTTGCAAAGGAACAGGAAGGACAGAGCCTGAACATTCACTGAGACCTGCTGGGTGTGAGGCCTGTTTTGAGCTCTGTCTATGAA
CAGAATCCCATTCAGCCTCAAAACGACCCCACGGGGCCGCTGACACTGAAAGAGAATGAAAGTCGAGGCGGAGGGTCAGTGGTCAAGCCAAGAG
CTGGCCCTCCCTGCTTCCTGCTCCAGCTGTGCAGACCTTCCTCCCGCCTTTGCCTGTGTGCCTGCTTTGCTTCCAAGCCATGAGAAGAGTCCTG
AGAGCCACGTATTCTTTTAACCTCAGTTTACCTTCGGACCAATCCTGAGACTTGTTACCAGAGGTTAAAGAAGAGATGCAGGGAAGAGGAGAAG
GTCATTCCGCTGGTAAGAGGCCTCTTTGACTTGGTTAAAGACAAACTGGGAAACCACCTCTGTCCAACCCGGCTTCCTCTCCCGGCCCCACCCC
CATGAGATCATTGTGCCCTTGTTTGCAGAGAGAGAACAGGAACCAAGTGTATCAGAGGGTGAACTCTGACTTCCTGTTTGGCCAGAGACCCACG
TGGATGTCCAGATCTGCTTGGCTTCCTGTGGTCGCTGACTCACAGTTTGGCAACCCTCCTGCTTGGAGGCGTTCCCCTCCCCTCCCCACCTCCC
CCTCCCCATCTTGGCAGCACCAGGGCTTGTCTGTTCTGCCTCCTGAAGATTGCCTGGCCTTGTGCTCTCTGTGTCCTCCCTAGAATGCGAGTTC
CAGGAGCGCAGGACCCAGCCCTCTTGTTCACTGCTGCATCCCTAGTGCTTACAACAGTGCCCGGCACGGAAACGGTGCTCATCCTTAGTGGTGT
TTGCCCACTGGAGAGATGCCATGCCCTTCTGAGTTCTGCTCAGGCCACACTGACTCTCATGGGGACTCTGCCAGTATAGGCCCAAGCTGTGCAT
CTCGCCCTGGCCCTTCCCTCCTTGCATCTCCCTTCTAAATCTGGCCTCCAGGCTGCCGCCCGGCTGATATTTCCAATACTCATCACCACTCGTG
CTCGCCCTGGCCCTTCCCTCCTTGCATCTCCCTTCAAAATCTGGCCTCCAGGCTGCCGCCCGGCTGATATTTCCAATACTCATCACCACTCGTG
CAATTTCCCAGCCCAGAACCTTTCAGTGCCTTGCTGCTGGCTAGAATGCAGTCCCACTTTGTAATACGGCTTATGACCCGGCCCCAATCTGCCT
CTCTGGCGTTATCATCCTCCATTCCCTACTTTCTATGCTCTGGCTATAAGGAACTAGTTGTGGCCACACACAAACAACCACATACACAGTGGAG
CATTGTAACCCCCCCCAAACCCACCCCCTGCCTTTCTGTGATCATGGCCCTTAGGACAGCATACTGAAACTATTTGTTTATACCTTTGTTCCCC
ATCCCTCACTAATCTGAGCTTTTTAAGATTGGCTCTTATTCCCTCCGTGTCCTCTGCATGTGCCTTGGGCCCTCGTGCCTAGTAGTTGCTCAGT
TAGTATTGAACTGAATGTCCCATGTGAGAAGCTGCCACTGGAGGGAGACAGAGGCTGGGCAAGCATTCAGTGGGGTGTCAGGGGCTCTGAGCTC
AGGATAGGCTGGACTGGGGGCCCTGGGAAGCCTATCCAACTCCTGAAATCCTGCTCAGCCTTGTTTTTCCTTGGTGCCAGCACGATGCATTCCT
GCCACGAGGGCCCAGGCGTCACTCTGGGCAGCTCCTTTGAAATCTTGGAGACAAGAATGCTAGGAAGATGGAATGTGGTGGAATGGGGCCAGGC
TGGACCATGCTGTGATGCGCACAGCAGCTCTGTGCAGTGACCCAGATGCTCCCCAAGCCTGGGGCCGTCCAAGCCGTGATTCCAAGCCATTGTC
TTAAGGGTTTGACCAGGTCCATGGGACACATCTGGCTCCTATTGCCAAGGGAGAGCTGGGCAGGAGATGTAAAGGCTGCTGCAGAGTTGACACG
CGAGCCTCTGATGGCGAGTGGAATCTGTGTGAGGAGCCATCCCACTCTGTGCCTGCCAGAGCACGAGTATCTGCTCTGCCTGGAATGCAAAAGC
CCCATGGCACAGGAGGGCCTGTGCCCCTTTTCCCTTGGGCTCCGCTGCATCAGGCTTTCTGAAGTTGGTGGTCTTTATAGTCTGGAGGGTCTAT
AGGTTACGAGTTGTTGAGGCGTAGAAGGCAAAATGGGTGAGGGTCACAGTCCTCTTGGTGGCAAGACAGAGCGGAGGGTTTAGAGGCTGCCCAG
GGCTGGGCCCCTGCCTGAAGGAACTGAGGCCCTGCTGCAAAAGTGGCAGTAAATGGGAGAAGCCAGGTGTGGGTCCTGCCTTTTTTTTTTTTTT
TTTTAGTATTTATTGATCATTCTTGGGTGTTTCTCGGAGAGGGGGATTTGGCAGGGTCATAGGACAATAGTGGAGGGAAGGTCAGCAGATAAAC
ATGTGAACAAAGGTCTCTGGTTTTCCTAGGCAGAGGGCCCTGCCGCCTTCCGCAGTGTTTGTGTCCCTGGGTACTTGAGATTAGGGTATGGTGA
TGACTCTTAATGAGTTATGCTACCTTCAAGCATCTGTTTAACAAAGCACATGTTGCATCGCCCTTAATCCATTTAACCCTTAGTGACACAGCAC
ACGTTTCAGAGAGCACGGCGTTGGGGGTAAGGTTATAGACTAACAGCATCCCAAGGCAGAAGAATTTTTCTTAGTACAGAACAAATGGAGTTCT
CCTATGTCTACTTCTTTCTACACAGACACAGTAACAATCTCATCTTTCTTTTCCCCACATTTCCCCCTTTTCTAATAGACATAACCGCCATCGT
CATCATGGCCCGTTCTCATGAGCTATTGGGTACACCTCCCAGACGGGGTGGTGGCCGGGCAGAGGGGCTCCTAACTTCCAAAGACTGAGCGGCC
GGGCAGAGGGGTCCTCACATCCTAGACGATGGGCGGCAGGCAGAGACACTCCTCACTTCCTAGACGGGGTGCAAAGCCGGGCAGAGGCTGCAAT
CTCGGCACTTTGGGAGGCCAAGGCAGGCGGCTGGGAGGTGGAGGTTGTAGCGAGCCGAGATCACGCCACTGCACTCCAGCCTGGGCAACATTGA
GCACTGACTGAACCAGACTCCGTCTGCAATCCCGCCACCTCGGGAGGCCGAGGCTCGCGGATCACTCGCGGTTAGGAGCTAAAGACCAGCCCGG
CCAACACAGCGAAACCCCGTCTGCACCAAAAAATACGAAAACCAGTCAGGCATGGCGGTGCGCGCCTGCAATCCCAGGCACTTGGCAGGCTGAG
GCAGGAGAATCAGGCAGGGAGGTTGCAGTGAGTCGAGTTGGCGGCAGTACAGTCCAGCCTCGGCTGGGCATCAGAGGGAAACCGTGCAAAGGGG
AGAGGGAGAGGGGGAGGGGGAGGGGGTCCTGCCTTTTGCCTTGGCTTTGATTCAGGCGCCAGGGGAAGGGCCAGTGTTTGTGCCGACGAACCCC
CAGTGGAGCAGAGAGGGTGGCTGGTGACACTTGTGCTGTTTTGCTCAACCTGGCCACCCAGTAATGTGGCTGCGTCTGCCGCTCTCAACTTGAG
CCAGGTTTGCTCTTGCAGGGTTCCTTTGTCTGAGTCACTGCCCATGGTCATTGACTACTTTGACCCCCTCTGCTTGACACCCTTGGTAGGTGGG
GTCTGCTCTGTCCACTCCCGACAGACTGCTTGTGTTTCCCTGGCAGGAATACCTGGAAGCAATTCACCATCTCCATGGGAGTGGCTCATCTAAA
GGCAGCCTTTTCCCCATGGCAGGCCCTGTTCTGGTAAGTAAGACTCCCTTACAGGGTACGAGACTGTTCTAGAAAGAGGAGTGACCCGGGGAAT
GTCAGCCACCTTGCAGACAGGGGAGGGCACAGTCCTTTAGAGTTTGACAACTTAGAAGAAGGAAGGTGGAAGGCTGGTGCTCTCTCCTCCCAGT
GCCTGTTTCTGCGCTGAGTCTCAGAGTGAGTTGGCCACACCCGAGGCTAAGGCAGTCTGTGCCAGTGCCATCAACCTGGCAGTTGTCACTGATC
CAGCATCTTCAGCGAAGACTCTAGACCCGGGTCTCAGTTTTCACCATTTGGGATAGTAGTTCCTCTGATGTGACCTTCCTCCTTCCGTGGAAGA
CACAGAGCCTCATTTCACCTTTGTTTGGTGACTCAGGTCATCAGAAGGAATGTCATTGTCTGGTCTGGGCTGTCACACAGTGATGCCCTTAGGA
ATGCTAAAGTGTGTGACTGTCTGCCCTGCCCACTAAATGTCTCTGCGTTCCTGTGTTTACTTTTTACAGTAAATCCTCCTTTCTCTTGCTCCCA
TGTGTCATTTCTGACTGATATTGATGTGTCTGTCCCGTCAGCATTTCCTTACTTCCCCATCTCCTTGTTTTCCAGTTTGCTGTTTCCAGATTTC
TTTACTCAGACAAATAAGCAGTGGCCCAGATGCTCCCAAGAAATGCGGAGTCTGGGGTCCCTCCGAGTCTTGATTGGAGGGAACCTAAGATGGT
TACATATACAACATGGGATTGGCTGTGAGGCAGGGTTGTGCGCAAGCTTTGGAGCATGGCTATACGAGAATTGTTTGATGGGATGTGAGGAAGG
GCTGTGCATGAGCTTTTGTGCGTGGCCATAGGAATATCTGTTTGGGCTCAAACACTTCTGCTTATGGCTCTTGCAGGCAGTGAGGTTGCCGGAA
AGTGAGATATTACTGTAGTAATTTTTTGTTTGTTTGTTTTTTGAGACAGAGCCTCACTCTGTTGCCCAGACTGGAATGCAGTGACGCAATCTCG
CTCGCTGCAACCTCTGCCTCCTGGGTTCAATGATTCTCTTGCCTCAGCCTCCTGAATAGCTGGGACTACAGGCGCATGCAACAATAACCTGGAT
AATTTTTTATATTTTTTCGTAGAGATGGGATTTTGCCACGTTGGCCAAGCTGGTCTCAAACTCCTGGCCTCAAGTGATCTCCTGCCTCGGCCTC
CCAAATTCTGGGATCACAGGCGTGAGCCACTGCACCCGGCCTACTGTAGTAGATTTTTAAATATAGTACAGCTTATTTTTCTCCCTCATTATGC
AAAAAAAGGAGGGTAGAAAGTGATGAGGGAAAAAAACAAATCATTCATGAGCTTACAGGGTCTCACTGTGTTGCCCAGGCTGGTCTCAAACTCC
TGGGCTCGTGTAATCTTCAGGTCTCAGCCTCCCAAAGTGCTTGGATAATAGACATGAGCCACCATACCTAGCTCTTGTAGTACATTCTTAAGTG
AAAACCTCATTGGAAATGTAGATTGCCCTAGAGCTGAAAGCGATTTGGCTCTAACCTTCCTTTTTATCTGCCTGCAACCTTCTGTGCCCTTCCC
TGCCCCCTGCTGCCTGCTTCCATCTGTCTGTCTCCAGCTGTGAAACGAGGATGCTGCTGAGGTCAGTGCAGCCTTGTGACCCATGTGTCTAATT
TCCAGGTGATTGAGGCTGACCACCACATGGAGAGGATGTTAGAACTCTTTGAACAAAATCGGGATCGAATATTAACTCCAGAGAATCGGAAGCA
TTGCCCATAGGAGGCAAAAGGTCTATGGCTCATGTCTGAAAAATGCCTGCTGCTGCCAAGTTAGCTATTGGGAGCAATCTGGAAAAACTTGCTC
CCAGGAGGGCTTTGTGTCTGGCCAGCTTGATTTTCCTAATGGTCTCATCTCCTTTGCTAGTGTCTTTGTCATGCCTCTCTGCCCCTCGTGGGTA
ATGACAACGGGACCAATGGGTTTGCCAAGCCCTTTGCTGTTCGCAGCCTAAAACATTCCCCCGGTGCCTCTCCCATGQCTTTGTGCTGCTGAGT
CGCTCTCATGAGCCCTTAGGGACAGCACCTGTTGTGTCCCTGACACCACGCTGGACCTGTGTACCAAATCGTCTAAGCCTTCATTAGGAGGCCG
AGGTAGGAGTCTTATATCCCAGGTGAGGAATTTGAAGCTCAGAAAGGTTGAGGGGCTCCCCAGAGGTCACACAGCCTGTGTGCAGTGGAGCTGG
CACCATTCAGACTTTCAGCCGACTCAGCAACTTTCCCTTGCCCTGGGCTGCCTCCTCCTGAGAGCTGTTCCCCACCGCCCTCCCTCTTCCGCTT
GGAGGCTCTCATGTCTCTTTGGCGAGAGCTGAGTGTGCGGAGCTGATACATTTTCCCTATAATATTGAGCAGTTCCCAAGGACAGTCAGCATTT
CTAGACTTCCACAAAATATGCTGCATTTGGCTGGAGCCCGGTGTTCAGTGGTTTCCCTGCCCGAGCTCGCTGCAGCCCCATCTACCAAAATCTT
CATGTGGACATTGAGATTCACATGCTGGCTCCTGAAGGGTCCTCAGTCTCCTTGGTGATTAGGTCCTGCTTGTAAGTGCTGCCAGCTCCATGTC
AGGGAAGTCGCTTTTGGTGCCTGGCTGGTTTGCCCAGAGCCAAGCTGGGGCAAGGGGCAGCCAGCCCTGGCTTCCAAGGCTCCCGTACTGTCTG
TGTCCTTGTATAAGGAGCTTTGCTCTTGGAATTACTGAAAGTCTGTGGCCCAAGAGAGAGACACAAGTGGCCTTAAGTCTTTTTGAAGTGTTAT
TTCATCCAGGGAAATCCCTCGAGCCATAGAGCCTGAATCATCTTTGTTGGCTCAGATAAATACCTTAGCTTCACTCAGCTGGACTGCATTGAAG
GCGAGGCTGCCCCTTGGATCAAGCAGAAAACAAGAGAAAGAAAGAACGTTCCCTTTGGGGATAGTCTGGAAAGTTGGGATTTGCAAATAAAGGC
TCTGGAAGCATTGCTGGTCCTGAAGCTTTGGAGGTGGGCAGAGAGAGCTTCAAGAAGACTAGATGCAAACCCTGGAAAGGATTAAGGCTCAACT
CTGGAGAAACAGGCCACAGCCTCTCAGAGCAGCTGTTGGCTGTTAAATAGAGGTAGCAAGGCCGCTCCCAGGCCCCTGTGAGTGTGGGCACTGT
GCATGCAATGCTCCGACTCTGCAGAGGTGCCAAGTGCCCCTGCTGGGCCAGTCCCAGAGAGTTAGGAAGTCAAGGCCTGCAACTCCTGGTTCTT
CCTGTTTGGACCAGTTCTTGTGCCATTGGCAGGATGAGAGGCAGCAGCCAGCCGGGAGCTGTGTCTAGCAGCACCTGTAGCCCACGTGCTGCTA
ATTAGCTGGAAAACTGGCGAAGGCAGAACCTTTGCTACCAGGAATCTTGACATGTGGGGTCTGTCTTTGAGAATTTGTAAATGAACAGTCCAAT
ATTTCCTCTCGGCTCATTTTGCACATCCATTTTGGGGAAATGTGATTTCTCTCTCTTTTTTTTTTTTTTTTTTTTGCCTAAGGCACAATCTCAA
GAGGTCCTGACACCACCTACCCATCATTTTTTTCTTGCTCTGTGATACCCAATACTCCTTCACCTTACCCCTGTTGTTGATTTTCTAAGTGCTT
CCTAGGCCAGTGATTTTAGCTTCTGCCAGCTGCTTTTGCCAGTAGATTAACGTGTTTTTATTTTTCATAACTCCGTGTTTCCTACGTCGAGTGT
ATGGGTCTAAGAGAGCCTGCTGTCCTCCCTGCCTTCCACCTTGGAGAGGAGGCTGGACGCATCAGCAGTGGCCAGGGCAGGTCGCAAAATCTCC
CAGCCTAGAGACCACACCTCAACGGCTGAAGCCAGCTTGCACAAGGGCTGCTCTCCCTCTGCGGCAGGCACAGCTGGTGGGGGAAAGGGGTCAC
AGAGCAGTCATAGACACCATGGACCAGGGCAGGAGAAGGGCAGATGGCACATGGGCACAACAGGGCCTTGTCCTTAGAGCACTGGGGGGTCATG
GCTGGCACGGGCATGGCAGGGGCTGGCATCCCTGTAGAGCCAGAGGCGCCACCGAGGGCAGTGACATTCCAGATATGTTGGGCTCACCTGATCC
TTGCTGTGAGACTGGAGTTCCATGGGGACATGATGTCAGTACACCGCAGAGCTGCTCAGCTGCTCTACCTCTCGCTCACTTTTTTGTTGCACAT
ATACATTTTCTTTCAATTAGCATTTATTTCAGCTTTTATTTAAGCTTTTTGACAGTACATGTAAAATATATGATTATAACCATTTAAAAATACC
TTATGTACCTGGTTTTTTTTGGAAACTAGATAGAAATATATTTATCTTTTACATAAAAGAAGTGTGTAGTGGGGTGGTCCAGGGCTTTGTGGTG
GCTTAGTGGCCATCGGGGTCCCAGGCTCTTCCCACCTTTCTCTTTTGGGTCCACCTCTTGGCTCCTGGCTTCCTTCCTCTGGGGCCTGACTGTC
CAGGATGGCAGCTGGAGCTCCTGCTCTGGGCACGGTTGGATCAGAGCCCACTGCTCCCCATCCACTCTTTTGATCCAGATGCTGGTAATGTCCC
TTTCCAAGGCATAACTAAGATAAGCTGGAAGGTTCTCACGAGGCTTTGCTAGAGGCCCTGGGAGGTGGGGGGAAAGGCAAGAGGGCAGTGCCCA
CCCATAGACCGGGTCACATGACCTGGCATCAGCGCCGTGGGGTCCTTTGGGCTTCACCTCCCTCTCCCCTCTGCCCCGACCTCATCCCACCCTC
ATCCTCACCCTCACCATTCCCATCCCGTACCGTAATGTCCCTTGCAAGGCCTAACTCTGTGAGGAATGAAACCACCTCATCCTTGTTCCAATAA
CATGAGTGACCGAATTTACAACGAGTGAATCTGGACCTCTGGAAACATTACCAAACCTTGTTTTTACTTTTCACTTTCTCTTTCTGCCCCTTTC
ATTTCCGTAGGAGCCCTTTACCTAGATGAGAAGTGTCCCCCCGCCTGGGGAATATATCAGTCAGAACAATCTTCCTGCAGACATGCACCATTAG
ACCCGAGTGACGGTGGTGCCATTTAAACCTCAGAGCAGGTAAAAGGTGGTCCTGAAACCTGTCTACCCACAGGGTGCTATGGAATCTGAATCAC
TTCTTTTTTCCTAGAGCCCTGGGGTGGGGAGCTCCCTCAAGTGTTCACATGTGTGTGGAATGAGGAACACCCATCTCCTTGGCCCTCTCCACCC
TGAAGAGTTAGTTATTAAAATAATTGGCAAGCTCTTGCAAATGTCAGTCATCCATTGTTCAGAATGGAATAGCAATAATACATCCCTGGCTGCC
CTGGGCTTGGCCAGGATTACTCACTGAAGGCCTCAGGGTTACTGGCACACACTTTCTTTTCCTAATAATCCCATCCCCTCAGCTTTCCTAAGGC
TAGAGTGAATTTCGTGTTCCTTTAGTTTACATAAGATGGTGAACTTGGCAAAAGCTATCATTAAACAGAAGCTAAGAGAAAGCCTATGTCGTGG
AATCCAGATCGGTATTGCCATTCACTGCTGTCCACAGAAGCTGTCTTGATAATTTCTTTCTGTGTCTTTTCTTTTTTTTCTTTAAGACTGTTGT
GTGGTCAAATAAACCATTTTCCATTCACATGGTGGAGTACTGTGCAGCTATACAGAAGAATGAGGAAGATCTCTCTGAATGGTTATGAAGTAAT
GTCTAGGACATAAAGTTATGTGAAAAATGCAATGCCCAAACGAGTGTCTATAGTATGCTACCCTTTATATTAGAAAGAAGAGAGGCCAGGCGTG
GTAGCTCACGCCTGTAATCCCAGCACTTTGGGAGGTGGGTGGATCACCTGAGGTCAGGAGTTCAAGACCAGCCTGCAACATGGGGGAACCCCAT
CTCTACCAAAAATACAAAAAAGTTAGCCAGGCGTGGTGGTGGGCACCTGTAATCCCAACTATTTGAGAGGCTGAGGCAAGAGAATCACTTGAAC
CCAGGAGTTGGAGGCTGCAGTGAGCCGAGACTGCGCCATTGCACTCCAGTCTGGGCAGCAAGAATGAAACTCCATCTCGAAAAATAAAAAGAAA
ACCGATAGAAGAAAATACACAGATGCAAAGGAAATATAGGAAGGACGCCCAGAGACAAAATAGGTTGGTGGGTAAAGGCTAGAAAGAAGGGAAG
GATGAGACTGGGCTACAGGGATGAGAAGGGAATGACACTTCTTTCAAATATCTTTTTATTTTATTTATTTATTTTTTTGAGACAGGGTCTCGCC
CTGTCACCCAGGCTGGAGTGCAGTGGTGTGATCTTGGCTCACTGCAAGCTCCACCTCCCAGGCTCAAGTGATTCTCGTGCCTCAGCCTCCTGAG
TAGCTGGGATTACAGCTGTGTGCCACTACGCCTGGCTAATAGTAGAGACAGGGTTTCCCAAGTTTGGCCTGGCTGCTCTCAACTGATATGCCCA
CCTTGGTCTCCCAAAGTACTGGGATTAGTGAACCGCTCCACCCAGCCTGGTAATCTCTTTTTCTATACTCACTGTTACATACTGTCATGTAGCA
ATGTTTCACATACCCTTCACATACTGCCAAAATAAATAAGAGCAACCAGGATGTAGGGAAAACAGAAAACAAAATTACAGATGAACTCCACCAT
ACCGCAGGTGAGTAACATAACCATACTGAAGGGGGCAGGAAAGAACAGAACTAACAAATAACTGTGGAAAACAGTATCTTTACTGGATATGGTA
AGAGTAAAGGCAAAAATTTCTGTACATAAAAGTTGTAGTATAGTTATTAAAAATGTTTCTCGACCATGCGTGGTGGCTCACACCTGTGATCCCA
GCACTTTGGGAGGCCAGGATGGGCGGATCACCTGAGGTCAGGAGTTAGAGACCAGCCTGGCCAACATGGTGAAACCCTGTCTCTACTAAAAATA
CAAAAATTAGCCGGGCGTGGTGGTGCACGCCTGTAATCCCAGCTATGTCGGAAGGCTGAGAATTGCTTGAACCCAGGAGGCAGAGGTTGCAATG
GGCAAAAAAAAATGTTTCTCACAGGAGTATAGGTTAGTGATTCTGAAACTATGTATACTAAAGTTGAACAAACAAGTAAATAGATTGTAGACTA
TGAGGGCTGGATTTCTTACTGTTAGTGAAAGAGGTTACAAATGATAGGGGTCTAGAATAAACCCCGTGGTGCTGGGTTAGAATAGGAGGTGTCA
GTCTAAACTCATGATTTTAAAATTTTAGGCTCTGGTGGCTCACTCTTGTAATCCCAGCACTTTGGGAGGCCAAGGCAGGTGAATAGCTTGAGCC
CAGGAGTTAGAGATCAGCCTGGGCAATGTGGCAAAACCCCATCTCTACAAAAAATACAAAAATTAGCCAGGTGTGATGGTGTGCGCCTGTAGTC
CCACCTACTTCGGACGCTGAGATGTGAGaTAGATCGAGTCCAGGGGTTCTAGGCTGAAGAGAGCTCTGATTGTACCATATGCACTCCAGCCTGG
GTAACAAAGGAAGACCCTGTCTCTAAATAAATAATATAATTATATATAQATGGATAAAGATAAATATATAGATGTGTGTGTCTGTCTGTGTGTG
TGTTTATGTGTGCTTAGTATACATACTTATGTTTCCTAGCTCTGTCCTGAGTGTGTCTAGAGCAAATGGCACCCAGTAACAGTGAGCACACCCG
GTACCCAGTTCTTGGTCTCCAAACATCATTCTCCTGCAAAAGGAACCAGGTCTCCTAGGAGATGTGGTTGATTCCAGGGATGCCACAGGGGAAT
GACAGGATGACCCTGGAATATCTTGTGGTGCTAGAAAAGAGTGCAGGGATGTGGGGAAATGATGGAGCCATGTCTTAGGGAATTGAGGAGCTTC
CCTGAAGTTCCTAACGGCCAAAGCTGGAACAGTTTGAGGAACAAAATAAATTATGTTAGTATTGGATTCTAACACATAGACTAAAATAAATATC
CACGAATCCATGCTGATATAAACAGTTGAAGATAAGTACAGGGAGAGATTAAAGGGAGTGCTTCCTTATGGTAGATTTCCAGTTAATACACACA
AAAGGCAAGAGGGAAACAGAGACTTGCCATTACATAAACACCACAGTTAAAAAATCACAGGCCAGGTGCGGTGGCTCACGCCTGTAATCCCAGC
ACTTTGGGAGGCCGAGGCAGGCGGATCACAAGGTCAGGAGATCGAAACCATCCTGGCTAACACGGTGAAACCCTGTCTCTACTAAAAATACAAA
AAATTAGCTGGGCATGGTGGCGGGCGCCTGTAGTCCCAGCTACTCTGGAGGCTGAGGCAGGATAATGGCGTGAACACAGGAGGCGGAGCTTGCA
GTCAGCCGAGATCGTGCCACTGCACTCCAGCCTAGGTGACAGAGGGAGACTCCGTCTAGGGAAAAAAAAAAAAAAAAAAAAAATAAATATATAT
ATATATATATATATATAAAATCACAGACAAGATTTACCTGTTGATACTAAAATTAGTGGACAGAAGTTTGAGGAAAAACAAGATTTGCATAGTT
TCTATTTCCCCTAATATACTTATCAAGTAAAAAGGGAAAGAAATTAACTAATTTGACAGTGGAGATGGAAACCCCATAGAAACCAGCTTAATAA
GTGATTAAGCTAATAAGACGTACTGACATCATGAAGCCTTTACTAAGATGGACTGAGGACAGCATCTTTTTTTTTTTTTTTTTTTTTTTTGAGA
CAGGGTCTCACTCTGTCACCCAGGCTGGAATGCAGTGGCGCAATCACAGCTCACTGCAGCCTCGACCTCCTGGCCTCAGGTGATCCTCCTCAGC
TTCCCCGGGAGCTGGGACTACACGTCTGTGCCACCAGCCTGGCTAATTTTTTTTTTTTTTTTTTTTTTTTTTTGAGACGGAGTCTCGCTCTGTC
ACCCAGGCTGGAGTGCGTGGCCGGATCTCGCCTCACTGCATAAGCTCCGCCTCCCGGGTTCACGCCATTCTCCTGCCTCACCCTCCCAGTAGCT
GGGACTACAGGCGCCCGCCACTACGCCCGGCTAATGTTTTTGTATTTTTAGTAGAGACGGGGTTTCACCGTTTTAGCCGGGATGGTCTCGATCT
CCTGACCTCGTGATCCGCCCGCCTCCGCCTCCCAAAGTGCTQGGATTACAGGCGTGAGCCACCGCGCCCCCCAATTTTTTGTATTTTTTGTACA
ATAGGGCTTTTGTCATGTTGCCCAGGCTGGACTTGACTCCTGAGCTCAAAGTGATCTGCCTGCCTTGGCCTCTGAAAGTGCTGGGATTACAGTG
TGAGCCATCATGCCTGGCAGCATCATTTGTTTTTTTGGTGTGGTATCCTTGCCAGAAAATGCATACTTGACTCCAGTATTAAGAAGCACCAGAC
AAACCCAAATGGAGGGACATTCAACAAAATAACTGATTGGAAAAAACTCTTTAAAAGCATCAAGATCGTGTTAGACAAGGAAAAAACAAGAACT
ATTACAGACTGGAGGAGACAAAGGAAAATATACAAATGCAATGTGGGATCCTGGATGGGCTCCTGGAATAGAGAAAGGATTTTAATAGAACACT
AGGAAACTTAAATAATATGTGTAGTATCGTTAATAACATTGTATCAATATTAATTTCCTGTTCTTGATGATTATATTATGGTTACATAAGATGT
CAACTTCAGTGCAATGTGGGATGCTGTATATGCTCCTGGAATAGAGAAAGGATATTAATGGAACACTGGGGAAACTTAAATAAGGTTTGTAGTA
TAGTTAAGAACATTGTATCAATATTAATTTCCTGTTCTTGATCATTATACTATGGTTACGTAAGATGTCAACTTTAGGAGAAGCAGGGCCAGGA
GTGGAAAGGAGTTCTTTGTACTATTTTGCAACTTTTCTCTCCATCTAAAATTAGTTAGAATAACGCCCTTCATGGGAAATAAAGGCAGAGCACA
ACACTGTAAATATTACAATCCCAAATGTTTAATATATGCGTATATAGAAAAAAGACTGGGAGGAATTGCAGGAATGTGGTGACATTGGGTATCG
CTGAATGGTGGAAATTATGGTAAGGTTTGCPTTCGTTTTCATATTCATCTGTACTTTCTAAGTTTTCTACTCATTTCATGGTTTAATATGGTGG
ATCCTTGGAAATAACGGTGAATGAGACAAGATGAAGTTTATCTCTCATGGAGCTTATCTTCTAATGGGGGGAAATGGAAAATGAATACATGAAT
TAAAATCTTTGTGCAGAAAATTAAGCTGATGTGATAGATGGTCAGAGGGGGCCTCTCTGAGAAGGTGACATTTAAGGGGAGATGTGAATAACAA
GGAAGAGAACATGGCAGGGTAGGGTAGGGAGACAGAGCAGAGGGAACAGTCTGTGCAGAGGCTGGGAATAGGGCCAGGCTCAGCATGTTCCAGA
ACCTTGCAGAAGCCAGGGTGGCTCTGGCTACAGCGGACAATGGGGAAGGGACCTGAGATGAGTTCAGAGGGAAAGTGAGGGGCTGAGCCTGGGA
CTTGATATGGCCTCGGGCACCCCTGGAGGGAACTGAACACATGAGAATCATGATCAAATGTAGGTGGTTTTTTTTTGTTTTGTTTTGTTTTTTT
TAACATTTTCTATTTTATTTTATTTTTTAAATATATAGAGACGGGGTCTTTCTGTGTTGCCCAGGCTGGTCCCAAACTCCTGGACTCAAGCAGT
CCTCCCACCTCAGCTTTCCAAAGTGCTGGAATTACAGGCATGAGCCACCACGCACAGCCCTAATGTAGGTTTAACGGAATCCTCCAGCATTCGG
GTGGAGCACAGGCTGCAGAAGGCTGGCAGCCTCCAGGGACGTGAGTGAGAGAGGATGGTGAATTTGGTTACTGTGGAAGGAGGGGGGAAGGGGT
TGGATTCAGACTCTGTTGTGAAAGAGAGGAGCTAGGAATTGGATGAAGGATGTGATAGAGAGAGAGAGCGGGGGGAGGAGGAAAGAGAGAGGAG
AGAGACATATATGTTGGTCAGGGTGATGTCTGCATTGCATTGGAGTGAGTGAGTGCATGGTGCACTCTATTTACCCAGATGGGCGATGCGGGAG
CGGCAGGCTTAGTGGAGAGAGGAAAAGTTCTGTTTTGGTTTGGAATAAGCATGCTTATTATGTTAATGGTCAGAAAAGAATGAATAAAAAAAAA
ATAATTAGCTCGCCTTTCCTGCCCCTGGATCCTTGGCCTAAGTTGCTCTCCCAGGTTCCTTTGCTCCAGGTCAGGTGCCCTGGAAAGATATGCC
TTGGAGTGGCTGTCATGGTTCCAAGCTGACCCAACCTCTTGCCTAATCC1CCTCACATTGCTCACCTGCTCTGCCTCCAGATCAGCCTGTTCTT
TTTTTTTTCATTTATTTATTTTAACAGACACCGTCTCACTCTGTCACCCAGGCTGGAGTGCAGTCGTGTAGATAACGGCTCACTCCAGCCTTGA
GCTCCTGGCCTCAATCAGTCCTCCCATCTCAGCCTCCTGAGTAGCTGGGACTACATGGCTGGCTATTTTTATTTTTTTTTTAATATTATACTTT
AGTTTTAGGGTACATGTGCACAACGTCCAGGTTAGTTACATATGTATACATATGCCATCTTGGTGTCCTGCACCCATTAACTCGTCATTTAAAC
ATTAGGTATATCTCCCAATGCTATCCTTCCCCCCTCCCCCCACCCCACAACAGGCCCCAGTGTGTGATGTTCCCGTTCCTGTGTCAATGTGTTC
TCATTGTTCAGTTCCCACCTATGAGTGAGAACATGTGGTGTTTGGTTTTTTCTCCTTGCGATAGTTTGCTGAGAATGATGGTTTCCGGCTTCAT
CCATGTCCCTACAAAGGACATGAACTCGTCATTTTTTATGGCTGCATAGTGTTCCATGGTGTGTATGTGCCACATTTTCTTAATCCAGTCTATC
ATTGTTGGACATTTGGCTTGGTTCCAAGTCTTTGCTATTGTGAATAATGCTGCAATAAACATACATGTGCATGTGTCTTTATAGCAGCATGATT
TATAATCCTTTGGGTATATATCCAGTAATGGGATTGCTGGGTCAAATGGTATTTCTAGTTCTAGATCCCTGAGGAATCGCCACACTGACTACCA
CAGCGGTTGAACTTGTTTACAGTCCCACCAACAGTGTAAGAAGTGTTCCTATTTCTCCACATCCTCTCCAGACCTGTTGTTTCCGGACTTTTTA
ATGATCACCATTCTAACTGCTGTGAGATGGTATCTCATTGTGGTTTTGATTTGGATCTCTCTGATGGCCAGTGATGATGAGCATTTTTTTCATG
TGTCTTTTGGCTGCATAAATGTCTTCTTTCGAGAAGTGTCTATTATATCCTTTGCCCACTTTTTGATGGGGTTGTTTGTTTTTTTTCTTGTAAA
TTTGTTTGAGTTCATTGTAGATTCTGGATATTAGCCCTTTGTCAGATAAGTAGATTGCAAAAATTTTCTCCCATTCTGTAGGTTGTCTGTTCAC
TCTGATGGTAGTTTCTTTTGCTGTGCAGAAGCTCTTTAGTTTAATGAGATCCCGTTTGTCAATTTTGGCTTTGGTTGCCATTGCTTTTGGTGTT
TTAGACATGaGTCCTTGCCCATGCCTATGTCCTGAATGGTATTGCCTAGGTTTTCTTCTAGTGTTTTTATGGTTTTAGGTCTAAGATTTTAAGT
CTTTAATCCATCTTGAATTAATTTTTGTATAAGGTGTAAGGAAGGGATCCAGTTTCAGCTTTCTACATATGGCTAGCCAGTTTTCCCAGCACCA
TTTATTAAATAGGGAATCCTTTCCCCATTTCCTGTTTTTATCAGGTTTGTCAAGATCAGATGGTTATAGATATGCGGCAATATTTCTGAGGGGC
TCTGCTCTGTTCCGTTGGTCTATATCTCTGTTTTGGTACCAGTACCATGCTG
HUMAN SEQUENCE-mRNA
GCCAAGTTATGGGTGCGTTCTGCCAGCGTCCTAGCAGTGATAAAGAACAGGAAAAAGAGAAAAAATCAGTGATCTGTGTCGAGGGCAATATTGC
AGGTGGGAAGACGACATGCCTGGAATTCTTCTCCAACGCGACAGACGTCGAGGTGTTAACGGAGCCTGTGTCCAAGTGGAGAAATGTCCGTGGC
CACAATCCTCTGGGCCTGATGTACCACGATGCCTCTCGCTGGGGTCTTACGCTACAGACTTATGTGCAGCTAACCATGCTGGACACGCATACTC
GTCCTCAGGTGTCATCTGTACGGTTGATGGAGAGGTCGATTCACAGCGCAAGATACATTTTTGTAGAAAACCTGTATAGAAGTGGGAAGATGCC
AGAAGTGCACTATGTAGTTCTGTCGGAATCCTTTGACTCCATCTTGAGCGGACATCGACGTGTCTCTTGATTTGATAGTTTACCTTCGGACAAT
CCTGAGACTTGTTACCAGAGGTTAAAGAAGAGATGCAGGGAAGAGGAGAAGGTCATTCCGCTGGAATACCTGGAAGCAATTCACCATCTCCATG
AGGAGTGGCTCATCAAGGCAGCCTTTTCCCCATGGCAGCCCCTGTTCTGGTGATTGAGGCTGACCACAAACATCCAGAGGATGTTAGAACTCTT
TGAACAAAATCGGGATCGAATATTAACTCCAGAGAATCGGAAGCATTGCCCATAGGAGGCAAAAGGTCTATGGCTCATGTCTGAAAAATGCCTG
CTGCTGCCAAGTTAGCTATTGGAGCAATCTGGAAAAAACTTGCTCCCAGGAGGGCTTTGTGTCTGGCCAGCTTGATTTTCCTAATGGTCTCATC
TCCTTTGCTAGTGTCTTTGTCATGCGTCTCTGGCCCTCGTGGGTAAATGACAAACGGGACCAATGGGTTTGCCAAGCCCTTTGCTGTTCGCAGC
CCTCACATTCCCCCGGTGCCTCTCCCATGGCTTTGTGCTGCTGAGTCGCTCTCATGAAGCCCTTAGGGGAGAGCACCTGTTGTGTGCCTGACAC
CACGCTGGAGCTGTGTACCAATCGTCTCAGCCTTCATTAGGAGGCCGAGGTAGGAGTCTTATATCCCAGGTGAGGAATTTGAAGCTCAGAAAGG
TTGAGGGGCTCCCCAGAGGTCACACAGCCTGTGTGCAGTQGAGCTGGAACGATTCAGACTTTCAGCCGACTCAGAAACTTTCCCTTGCCCTGGG
CTGCCTCCTCCTGAGAGCTGTTCCCCACCGCCCTGCCTCTTCCGGTTGGAGGCTCTQATGTCTCTTTGGGGAGAGCTGGCAGTGTGCGGAGCTG
ATAACATTTTCCCAATATTGAGCAGTTCCCAAGGACAGTCAGCATTTCTAGACTTCCACAAAATTATGCTGCATTTGGCTGGAGCCCGGTGTTC
AGTGGTTTCCCTGCCCGAGGTCGCTGCAGCCCCATCTACCACATCTTCATGTGGAAATTGAGATTCACATGCTGGCTCCTGAAAAGTGCTCAGT
CTCCTTGGTGATTAAGGTCCTGCTTGACTGCTGCCAACTCCATGTCAGGGAAAGTCGCTTTTGGTGCCTGGCTGGTTTGCCCAGAGCCAAGCTG
GGGCAAGGGGCAGCCAGCCCTGGCTTCCAAGGCTCCCGTACTGTCTGTGTCCTTGTATAAGGAGCTTTGCTCTTGGAATTACTGAAAGTCTGTG
GCCCTAAGAGAGAGACACAAGTGGCCTTAAGTCTTTTTGAAGTGTTATTTCATCCAGGGAAATGCCTCGAGCCATAGAGCCTGAAATCATCTTT
GTTGGCTCAGAAAATACCTTAGCTTCACTCAGCTGGACTGCATTGAAGGCGAGGCTGCCCCTTGGATCAAGCAGAAAACAAGAGAAAGAAAGAA
CGTTCCCTTTGGGGATAGTCTGGAAAGTTGGGATTTGCAAATAAAGGCTCTGGAAGCATTAAAAAAAAAAAAAAAAAAAA
HUMAN SEQUENCE-CODING
ATGGGTGCGTTCTGCCAGCGTCGTAGCAGTGATAAAGAACAGGAAAAAGAGAAAAAATCAGTGATCTGTGTCGAGGGCAATATTGCAGGTGGGA
AGACGACATGCCTGGAATTCTTCTCCAACGCGACAGACGTCGAGGTGTTAACGGAGCCTGTGTCCAAGTGGAGAAATGTCCGTGGCCACAATCC
TCTGGGCCTGATGTACCACGATGCCTCTCGCTGGCGTCTTACGCTACAGACTTATCTGCAGCTCACCATGCTGGACACGCATACTCGTCCTGAG
GTGTCATCTGTACGGTTGATGGAGAGGTCGATTCACAGCGCAAGATACATTTTTGTAGAAAACCTGTATAGAAGTGGGAAGATGCCAGAAGTGG
ACTATGTAGTTCTGTCGGAATGGTTTGACTGGATCTTGAGGAACATGGACGTGTCTGTTGATTTGATAGTTTACCTTCGGACCAATCCTGAGAC
TTGTTACCAGAGGTTAAAGAAGAGATGCAGGGAAGAGGAGAAGGTCATTCCGCTGGAATACCTGGAAGCAATTCACCATCTCCATGAGGAGTGG
CTCATCAAAGGCAGCCTTTTCCCCATGGCAGCCCCTGTTCTGGTGATTGAGGCTGACCACCACATGGAGAGGATGTTAGAACTCTTTGAACAAA
ATCGGGATCGAATATTAACTCCAGAGAATCGGAAGCATTGCCCATAG

TABLE 35
MOUSE NOMENCLATURE
ICSGNM Nupr1
Celera mCG22384
HUMAN NOMENCLATURE
HGNC N/A
Celera hCG1745228
MOUSE SEQUENCE—GENOMIC
TTGCAGATCTAGACATTGACATCTGGTCTTCTCAAAGCTAAGGGCAGGCATGCTCTGGCCTTGAACTCCATGCTCAGCCCTAGTCCTTCCTTTC
TTCTTCTTCTTCTGTTAAATGGGGTTTGATTTCTTTGTCTGATAAATGAGGCTGGGAGGGAGGGAGAGATGATGACTCAGCAGTTCAGAGCTCT
TACTCTTTCAGAGGACCCAGGTTTGATTCCAGCACCCACCTGGTGGTTCACAATTGCCCACAATTCCAGGGTGCACATGATACAGACAAAATTC
AAGCAAAATAGTCACACACACACATAAAATACACTAAATAAATCTTTTCTGAAAAGACTAAAAGGGAATTGTGATTGCTGGTCTCTTGGGCTAC
AGCCAGGAGCTGCATGTTTATTCTGTATTCTTCTAGTTTGTTGCTCTATTTGCCCACTATTTTTTTAGTTACCAGGAAAAATGTATGATAGCTA
TATTTTGCTGAAATATGTATAACGCATAGGCACTTGTTCTTAAATTTCATGCAAATAGTATTACCTGGTTGGTTGGTTGATTTGAGAAAGCCTT
GATAATCATGTAGCCCACTCTGGCCTGGAACTTGCTATGTAGCTGTTGTGTCTGTCATTGTCCCAGCCAAGCAACTGCAGTCTTGAGGAGTTCA
ACTGTACCCCCTTGCAAGAAAAAAAAAAGTATCCTAAGCTGGGCTTTCTTGACTCTGTACACCTCCTCATGAAGGAGTGAGGAGGCTTGTGAGA
CCCTCACCTGTTCCTCCAGGTAATAAAACCCCAGTCCCTGTTGTTTGCAAGTCTGCCTTAATTGCACATGACCTGGGAGGAGGCTAGAGTATAT
AGGCCAGGGGAGCACAGAGGAAGGGGAGCCCATCCTTGTGGGATGGGTAGGAGAAGGAGCATCTATGCCCAGGTAGGTAACACCCCTTACCTCT
CTGCTCTGGAAGGAAGCCCAAGAAATGCATTGTTTCCCTAGGGTGAGCTTTGGCAGAACCACGCCTTCCTTTGATGGGGGACCTTAAAAGAAGA
AGTAGACTTTGTGTTTATGTCTCCAGGGAAGGAATTTTAACCAAAGTAGCTGGGATAAGCTTGAACTCAGGTGTGAACTACCACACCCAGTTTA
TGAAGTTCTGGAGATAGAACCAAGAGCTTCAGGTATGCGGGGCAGGAGCTCAGCCAAGTAAACTGCATCCACAGCCCTCGCCTCTTTACTTGCT
GTGAGAAACTCTGCACGTAGAACAGAGTATCAGGGAGGTTTGTACAATTCGGGATCTCACCAGGATCCCCTAAGCACGTGTTAACAGCAGCATC
CAGTCTCAGAACCAAGAGAAATTAATTCCCAGTGCAGCCCTGGGTTTAAGGGTTTTATGGACCAGGCACAAGCTGTAATTACTGGGCTGTGCTA
GGTATTCCTGGCAACCACCTGGAAAGTTATAAAGGTGTAATGAGTGATAAATAGCAATCTCCCTCCCCCCAAACAAAACAAAATCCCAGAATGG
ACTACCTGGAGGCCACAGGATGTGGAAACTTAACTCTTCATTTCCCAATGAGTCACTTTTGAATTCCTACTGATCTGAGTGGAGTTTAAAATTT
TTCCACAAATCCTTTTCTCAGGTTTCTATTCTTTCACCCAGCACCTACTCCTCTCTCCCTTCCCCTAGTGTTTACATTTCCTTTCTTTTATTCC
TGCAAGTTCCCAGCCAGAAACCCTTACCCTTACTCCATCATCACAGAGTCTCCTAGGATCTTGGAGGTCCTCTGTGAGGTCTGCCTCTTCAGAG
CAGAACATCTAAAAGTTGGACACACAATCCTGAGGCACCTTCTCAGAGTTTGTGTGTTTTGGGTTGTCATTTGCTGAGCTGGGTGTTCAAGACA
TGGAGATTAAGCAAATTGCCTGCCCTAGTGAATAACAATACTATCTTCAAGGCGCTGTGCTAATAAGCTCCCCTCACCCCCCAGTTTTTTCTTC
TTAAGACGGAGTCTTAGCTGGGTGTAACGGCCTGTAATCCTAGCACAGAGGCGGTAGCAGAATCAGGAGTCCTAGATCGGCCTACGATACATAG
TAACCCGAGTCTTAAAAGAAAAGAAAGCATGCTGAGTATGGTGGTGTATACCTTTAGTCCCAGCACCCCAGCACTATGGAAGTAGAAACAGTCA
GATCTCTGTGAGTTTGAGGCCAGCCTGATCTATATAGTGGGTTTCAAGATAGTAAGGGATATGTAGAGAGAACTTGTTTCATATACATACCCAC
AAGCAAAGCCAGGCTAGGTGGTGACTGCCTTAAATGCCCCAGCACTCAGTGTTAGCTCAGGCTTAATGAGATGATGTGAAAATAAAAAGGGAGA
GAAGGATGAGGAATGGGAGCTTCCCTTGCCAAGGAGACGCAGATCTGTTCCCTGCAGTCTCATGGTAAATAAAACTGAGGTTTGGTTTGGCTTT
TCGAGACAGGGTTTCTCTGTGTAGCCCTGGCTGTCCTGGAACTCACTTTGTAGACCAGGCTGTCCTCAAACTCAGAAATCTGCCTGCCTCTGCC
TCCCGAGTGCTGGGATTAAAGGCGTGCGCCACCACGCCCGGCACAACTAAGCTTAAGAGAGGTGGACTTCCTTGTCAGGCAGCTGTACTCATCA
GAAGCTCAGGCCAGAGTTGCTCAGATCACCTGAGGCTCTTTGTTTCATGGACTCCTCACTCCAGGAAGCAGAGGATCTCTGTGACTTTAAAGCA
GGTGTGAGACAAACCAGGAGCTATATTGTTATACATTGTTATTAAGTCTCCATTCCTTACAAGAATAAGTTTCTCTTTCCTAAACCTAAAGTAT
CCTGCAAACTGTTTTCAATCTACCCTGAACCGGGCCCACTCTTTTGTCTTGATAAACCCCTGAGCCTGTGATAAGTGATCACCCTTTTGCTTTA
TGAAAGTGTGTCTTCCCAGGTACCTGATGGCTCCCCTTTGACATAATGTGTTCTTGCTCTTATTTTTATTGCCTCCTTTCTAGCAGCCAGTAAC
CACAGCTGTTTCCTATTACACTTATCTACCCCAAGGCAGCCTTGAGCCCTGAGTGCCCCCATTCTCTGACCAGTGCTAACAGCCATTATCTTTA
CCGCTGTCTTTTCTTTTCCTTTTCTTTTGTTGCCCCCCCCCCATTTTGTTTTGCTGGCCTTAAATTCAGAGACCTGCCTGTCTCTGCCTCTCAT
TATAGGCATGAGCCACCACTGCCCAGCATGGACAGCTGTGTCTATCAACCCTAACCCATTCCTCATAAAAGGGTTCCCAAAAGCCAGTGAGGGG
CTGTTCTCTGAAACCCCAACTTAGGGTAAGGCAGCCATCTGGCCAGTCCCTTGTGCACTTCTCAATAAAGCTCTCTTAATCAGACAGTTGTGGA
AATGGTTTTTTTCTCAGATCTAACACCAGTTGGTTCTACACAGTGAATTCCGTGCCAGCCAGGGCTACAAAGTGAGACTTTGTCTCAAAGCAAA
AGAAAGAGAAAGAAAAAATAATGTGGAGAGGAGGAGACAAGATCTTGCTGTGTGTTCTAGGCCAGCCTCAAACTTGTGATCCCCTTCTTCAGCC
TTCCAAACAGCTGAGATCACAGGCATGCACCACAGCACCAAACTAAGAGTTCTGCCCCTTGGAAGTTAGAGGGACGGAGTATATATGCATGTGC
ATATGCATGTGTATGTTCAAGAGCGCCTTGGGGGCAGAGGGTTGGGGAAGAGGCACACAAGTATTTCACAGAGCTCGTATATAGGTTAGAAGAC
AATCTATGGGAATTGGTTTTCTCCTTCTGCCACGTGGGTCTTGGTAACTGACCTCAGATCCTCAGGGCTGACAGCAAGTGTTTTGATGTGATGA
GCCATCTCATGAGCCCCAAGAATTATCCTTTTAAAGCTCCCAGAGACCCCACTGAAATCTACGAATGGGGAAACAAAGACACAGAGAGGTTAAA
TAACTTGTTTGATGACACACAGCCAACAAAGGATGGAACTAAAACCTTGGCTCCAGGCAGCCAGATTCCAGAACCTTCTCCTTTAACCACTGTC
TAATCAATAATAGTTTGATTCTAATAAAGACAAATTTCTAAAAAGTGAAAAAGATACTTTATTTTGTTTGTTTTCCTCTGTGTAGCCCTGGCTG
TCCTGGAACTGGTTTTGTAGAGCAGGCTGGGCTTGAACTTTGAGATCTACCTGCCTCTGCCTCCAGAATGCTGGGGTTACTGGCGTATATCATG
CCTGGTAAGATACCTTGTTAATGCTAAGTGGATGCTGTGCAGATAGTAAACTAGAGCAAGGTGGCGCAGGACTACAGCTGCTGGTGTGTGTGGA
GGTTTTGTGTTTGGCCTGGGTTTGTCTGTTATTTGTTTTTGATAAGGGTTCATTATGTGGCCCAGGCTGGTCTTAAACTTGAGATCCTCCCGCT
TCAACCTCCTGAGTGCTTGGGCTACAGGCATGTGTTACCATGGTTGGCATATATGTGGTACTTTGAACAGGGGTGTGTACTGCTACATTGAGCA
TCTATCTCCCTACGTCATAGAGCTTTCAAAAGGTATATGATGTTCTCTGCAGGCTCTGTACAAGGCCTGGTCCAGGGAAGACTCAAATGCCTGG
GAAGGAAGGGAGTGCAGAGGGAGAGGCCTGGCAGGTGACTCAGTGCAAACCCTTGTCAGTGTTTGCCCAGCTGGGGTCTGTGTGTAGATCATGG
GTCCTGTGACTTTGAAACAGATACCCCTGGAGCCTCTTTGAGGAATAAGGAAATAACATGATGTCACATGTTGGCTCTGGGCTAAGCCCTGTCA
CACACATAGAGGTGTTCTCATTGTCCTGGAATTACACAGGAACAAACTGAAACTTGGAAGTTGACAGCATGGTGTGCCATGATGAAGCAGGGAG
TTCTGTGTGCCTTTAGGAAGGAAAATAAAGGTAGGACAGTTGTGTGTCCATGTGTCCCACATCCTGACTGTCCTGCTGTCTTCCTGGACATTCA
GGACACTAGTAAGCTTTTTTACAGTCTGTTTTCCTATAACCTGATGTCAATCTATTTCAAATGAGAGGAAATATTTTCTGTCATGATGATAAAG
GACAGGTAACAAGGACATCATTTTCGGTACCTTTGTCTTGATACGTTAGCCCTGGGTTTTGTTTCTTTGCTTATTTTAACTTTTTTTTTTTAAA
TATTGATTTGAGGTCAGGGTCTCCCTGGGTAACCCAGGCTGGCCTTGTTAATCTTAGTATCACATTAAAGAACTTCACAAATCAGACATAATCC
AGCAAAGCAATTTCTTTTTATAGAGTCGTGACCCAAACCAGGCTAGCAAGCACACCGGAGCAGGAAGTTCACTTAATTGTTATTCTAACGCAAG
TATTGACTGAGTCACCTCTCATTGGTGCAGCTTGGCCTCATAATCTGAAAAGACTGGCTAATTTATAATCACACACAAAGGAATTGAAAGAACA
GAGGGCTGTGTGTCATCACATTCTGGGACTTAGTGAGGTTTTGTATAATAAGAGTTTCAGTTGGGCATTTTACATTGTGGGGAGATTTATGGCA
CACTGGCTCTTGGTGGGCTAACCACATTAGGTGGAAAAAAAAAGATTCTCGGAGCCTTGGCTTTGAGATCTTGTGGTGGTGGGATTTCAAGCCG
GCACCACCACACCCAGAGCTTTGTTTCAAGGTTTCTTCCCTTGTGTTTATTTCCACCTTGGGATTAGACCCAGAGCCTCAAACCTGCTACTCTC
TACTCTCTGTCCCTAGCCTTCTTCTAACTTTCTGCCTTGGGACAGAGTCTCACCAGGTTTCCCAGGCTAGACTTCAACTCATTTTGTAGCCCAG
TCAGGCTTTAAGTTTGCCTTGTTTCCTCAGTAACTGAGATCTCTCATTCTCAACACCAGAGTCAGGCTCCATAATGTTATCCTCTCTGGAGTCT
CCTCATAAGGTTTCCAATTAGCCCAGTACTGCTCTGATTCAAGCAACTTGCTGGTTTTACTCTCATATCCTAGGTTTACATTTTGATTTACCCT
ATCTGTCTGTCTGTCTGTCTGCCTACTTACCATATGTTTGTGTGGGACTGTGATGTAAGCACTGCCTCTGTGGGCATCTGTGTGTTTGTACACA
TGTGTGTGTGTGTGTGTGTNNNNNNNNNNNNNNNNNNNNACACGTGTGCACCTGTGTGTATGCTCGTGTGTATACCTGTATGTGTGTGTGCCTG
AGTGTGTACACCTGTGTGTGTGCATGCCTCTGTGTGTGTGCCTCTGTATGTGTGTGTATGTCATTAGTATTCTGTCTAAGCTCCACCCCACAGT
TACCTGGCAACAGCCAGGTAGGCCTGACTCACTATAAAAGGGGCTGCTTGCCCCCTCCTCCTTCTCTTGCTCTTCCCTTCTTGCTCCTGCTGTC
TTTCCTCCCTACCCCTCCAGTCCCTTCTCCCCTCTCTTTCCATGTGCTCATGGCCAGCCTCTACTCCTGTTCTCCTCTACTTCTCTCTCTCTAC
CTTTCTCTGCCTCTACTACCCTCTTAACCCCACCCCCACCCCATGCCCTGAATAAACTCTATTCTATACTATACCATCCTGTGGCTGGTTCCTC
ACGGGGAAGTCCCTTAGCATGAGCCCACGGAGGCACCACTTCCCCCATACCTCACCACACCTCCATAGAACATATCTCCCTTTTCTTTATTTTT
TGTTTTTGTTTTTATAAACATATCACACGTGGTAGCAGGAGGATGCTTTATTCCCTGAAACAGGCTCTGTCACTGAACTTGGAGCTAGGCAGCA
GCTAATAAGCAGTCCTTCTGTCTGCTTCACTAGTGTCACAGTGCTGGGGGCAGAGGTGTGCACATGGCCGTGCCTAACTTTTAACCTGAGTGCT
GGGATCTTAGCGCGGGCCTTCTTGTGCAGCAGTGCTCTCACCCACAGGCCATCTCTCCAGGCCCTGGGCCGGAGCTTTAAAGCAGCGCTGACAC
AAGAATCTGGAAAACAGATGCTTGGTTTTGTTTATGTTCAGGGGAGAAAGCCGCAAACAGAAACACAGCCTCTTCTTAACCAGGGCAACCAGGG
CCTGCAGCAGGCCTGGGGTCACATCCGAGCCTTGTGCTGACACGGAGGGAGGTTCACAGCCTGTGGACGTGGGCAGAGGACTTGATGTCTGGCC
AGGGTTGAGAGGGTTTGGTTTTTCTGGAATCCAGTTTTGTTTTGTTTTTAGATGTGGAATGTTTTGTCTAACTCCCTGCCTGGAGACAGCAATG
CAGATAGAATTGGTGTGGGCACCCTTTCTGGGCTAACATGACTCACAGGGTTCATATCCTTCCATCAAGAGTAGGAAATTCCCCTTTCCTCCGA
GCTCAGCAGCTCTGTGGAAGAGGAGGTGGAAAGATTGTAAGAGCCGGGGAGTGTGGATACCACGAAGGAAACAGTGTCTTCCAGACACAACAGG
ACTGATTGATACACATATGAACTCAACACGCTGGCAGCCTGCCTGCGCCTGCCCAGGTTCAAGCCACATGGGGTCACAGCACTGAGAGGGGAAA
GTGGACATGGACTCCCACCCCTAACCAAATTGACACCTGCTTACAAAGGAAAACTTAGTTTCCTCCAATAGAGTCTCACTGGCTGCATTAACCC
CAGGTAAGGGTCAGCCCCATGGCCAGCAGGAGATGCCAACACAACAGTACTTTTATCGGCTTTCTTTTCTCACGGTGTTTTCATCAGACATGTT
TTGGTCTTATTGGTCCTTTGTTTACATATTATAGTTTCTGATTTTGTGTTTTTATGGATTTGTGTGTGTGTGTGTTTTGTTAGGACTCAACCCC
TGGACAAATGGCTAACTGAAGTGCCTATTAACATATCAGGGCAGATGCTGTCAGCCTGCCTCTCTCAGCATTGCCAGCACCTATTACGGAGCTG
TCCTGGCTGGTATACCCCACATGCTACCCTAACTCTCCCTTCCCACAGTTTCTCTGATTCCTAAAATAAGATGACTATGCTAGTATCTTTGCCT
TTTTATTACCTCTTGGTCTCTGGGTTCTCAGGTTTTTTTGTTTTTTTGTTTGTTTGTTTTTTGCCTCTTGGCCACTTGGTCCAAGGTTCCTCTG
TCGACCCTCCTCTCTTCTCTTCCCCCTGTCTCTTTCTCTCTCTGTCTCCCACAGGCCCTGTTAAGTCTGCTGGCCATGTTCACTCTGGACTCTT
GCAGATAGCTCTGCTTGTGCTCTCCCTCATGTCTACACTAAACCTTCTCCACCATACCCAGGAGCCGCATGGCCTCTGCTTTTTAATTTTTTCA
TTCATATTTCTGACGACGACGACGGTGACAATGATGATTCACCTGTTCGTTTCCTATAGAGTAAAATTTGGGGGCATGGGGAGGGTCTGGGAGG
AGTTGGAGAAAAGAAACCTTGATCAGAATACATTGTATGAAAACTTTTTTTAAAATAAAAAAAAAGTAGAAAATTTCATTTCACATGCTTTTTA
TTTGAACTTAAAATTCATACTAATTCTTATCAAACAACCCAAGGGAAGCTAGTATGGACTGAAAAAGCTTACTATTAGTTCACACTTGATTCAT
ACTGGCTGGGTGTGGTGATGCACACCTTTAAACCTGGCACTCAGGAGTCAAAGGCAGGTTGATCTCTGAGTTCAACACTAGACTCAAATCTGGC
CTACAGAGCAAGTTCTTAGCCAGCCACAGCTAAAACAGTGAGACTGCCTAAAAAACAAGGCAGGGGGTATCAAAGCACAAATACCTGTTTATTT
CATTCTGCCCTCAATATATTATTTAAATTTTAGAAGTCACACGTATTTATTGTGTGTGTACATGGATAGGAATGTGAGGGTCAGAGGACAATTT
ATGGGACATTTTTCTCTCCCATACTGTATGGGTCCTGGAAACCGATAAACTCAGGTTGTCAGGCTTGGTGGCAAGTACTTTACCCATTGAGTCA
TCTTGCCAACCCACAATATTTTATTTTAAATTCCTTTTCTCCACTCTTTTTTTTTTTTGAGGGGGGAGGGTCAAGGCAAGGGCACTGTATGGGA
ACCCATGGGAAGTTGTGACTAATCCTTACACTGTTCCCGCTTGTGAAGGTTTTCCAGGCCTCATGAGTCTGCAACCTCTCACCAACAGAGCCAG
ATAGAGTTATCCTCTGTTAAAGGGGGAACCTGAAGCAATCAGAGAGGGAAAGTGACGTGTCCAAGGTCACACAGCAGGAAACACTGGATCTGAG
CTGACTTGTGATCCCAGCATTTAGGATGAGGAGGCCAGAGAATTGCAAGTTCAAGGGCTAACCTGGGCTCCATAGAAGGATTCTGTCAGAAAGA
AGGGTAGAGGGAGGTGGATCAGGAATCAGAGTGTGGAATCCAGGGTATTTGCTGTCTCTATCAGCTAGCTATCCTCCATCTTGCATGAGGAAGA
AGTCCCTGGGGACTCTGTTGAAGGGGACTTTTCACAAAAATAGAAGCCTTGGGTGTGACAGGAACTGCTGACCAAGCAAGAAGTGGGTCAGTGT
CCCTCCTCCCTCCCTTTTCAGTATGTATGCTGCCACTACCTCCTGAATCTGAACCAGACTACAGTGTGTGTGTGTGTGTGTGTGTGTGTGTGTG
TGTGTGTGTCTGTCTGTCTGTCTGTCTGTCTGTCTAACTATGACCCAGGCCCAGGAGGGGCTTGTGGCAGAGGCTGAAATGATAGGAAGGACTG
GCCTATCTGAGGCCCTCTGCTCTCCCTGGCCAATATCCCAAAGACTGTGCCCAGATCCTGTCTGCTTCCTCCGCCCCTGAGGCGAGAGCTTTCC
ACGTGGAAAAACACAGCTGTGCTTGGGTGAGTCCTGTGAGGGAGGAGGGAGGGGAGGGTGCTCCTCTCTGCTCAGGACTGCTGTCTATGGGCCC
AGTTCTCGGAAAGGGCCAGATTGCATCAGACAGGGCTTGGAGGGCGGGGCCCAACCCCGTGGTTTAGGGAGGAGACAGCGTTATAAGTACGGTG
AGAAACTGGGAGAGGGCAGACATTCAGACAAGGAGACACAGGCAAGACTTTGGAGAGAGCAGACTAGGCATAATGGCCACCTTGCCACCAACAG
CCAACCCTTCCCAGCAACCTCTAAACCTAGAGGATGAAGATGGAATCCTGGATGAATATGATCAGTACAGCCTGGCCCATCCCTGTGTCGGTAA
GGTTTTCGTGGTAGCTCCTTGGTCAGCCCATCTGCTTCTCACTGGGTACCCCGGTTTACCTTTCCTAGCTAATCTATTCCCAACTCCCAAGTAA
CCCCTTCTAGTCTCATCTACTGAGTTCTGTCTCCCTTAGCCCCAAGAACTCACAAGTGAGCATCTCTCATTCTATACTTGAGTAAACAGCAAGG
ATAGAGTGCTTCAGACGTGAAGACGTGACCGCCCCACTCTGGCCATTTATATGCCTGTCCTAACCTCACACACCTCCTCTACCTCCTGCTCCAC
CAGTCGGGGGAGGTCGGAAAGGTCGGACCAAGAGAGAAGCTGCTGCCAATACCAACCGCCCTAGCCCTGGCGGGCATGAGAGGAAGCTGCTGAC
CAAGTTCCAGAACTCTGAAAGGAAAAAAGCCTGGCGCTGAGACGGAGCTGGAGGTGAGTCGGGAGAGCCGGGCCTAAGTGTGCAGAGCGGGAAG
CGGGGAACCAAGAGCTGGTCCTGGTGAGATGGCGCTGCAGGTAGAGGCACTTGCAGGCAAGCAGGACAACCTGTGCTTGATCTCTGGGACACGT
GGGATGAAACCACGTGATGAAAGGGGAGAGAACCAATTGCAAGCTGTGTTCCCGAGGGAGCCCTGCAGATAGATGTCCAGGCAGGGAAGTGATA
AGAATGAAAACCCCATTCAGACTGACTTCCATCAGCTTGGATCAGATTTCAGGGCATCTAACGCCAGGCGGTTCACTGTCAGCATATTTAAAAC
ACAGTACAATTTGGGAAGAGGTTTCTAGGCAACAGTCAGTCCATCAGTTCAGTTCCAGGAGCACAATAAAGCTTGAGGTCATCAGGCAGATGTG
ACATCTTTCCTAAGAGTGGGTAATGGTCTAGGGAGGGGAGAGTCTGGGATTATCATTCTGGAGAACTTGAGTGAGGTCGGCTTCTATAGCAAAA
GCTGGGGGAGGTCTGACTCCACACAGAGCCAAAAGCTCACCGGGTTGTTAACAAAATCCACTCTCAGATTTCTGAGTGGAATGCAGGTATTTGA
CTTTATCTCCTGCATTGTGGAGACACACGGCATGATTACTTCTGGTTCTTAGTGCTTCTCTTGAGTACAAGGACCACTTCTGTACCTCCAATGT
CCACCGACTTCCATATGCTCAAAGTAGCACATGAGAATACGCATGTGCACACATGCATGCAAACACATTGGCATATCAATAAATGAAAAAAATA
ATGTTGGCCGGACAGTGGTGGCGCACGCCTTTAATCCCAGCACTTGGTAGGCAGAGGCAGGTGGATTTCTGAGTTCAAGGACAGCCTGGTCTAA
GGAGTGAGTTCCAGGACAGCCAGGGCTATACAGAGAAACCTGTCTCGAAAAAACAAAATACAAAAAACAAAAACCAAAACCAAAACAAAACAAA
ACAATAGTGTTGTGACACTATTTTTATTCTACATCTAAAATAATATGAGACCCGTATTTGACTGAGCTCCCTGGTAACCAGAGGTGGGGGGAGT
TCCCATGGAGCCTTTCCAATGGAGAGGAAGTTTGGTTTATGGAAAGGGAAACTGAGGCAGATAGAGTCAAGTACTTCTCTAAGGTAATAGCAAA
TATGAGTAGGAAAGGTGGAAGTCAAACTCTGATCTCTGTCCCTTTCGTTGCCTGCTTTCCCTCTGCACAGATAAGGCCAGACCACAGACACCAC
ACCCAGCAATGGATACAGGACATCACACCCAGCAATGGATACAGGACATCACACCCAGCAATGGATACAGGACCTTGGAGAAATTAGGAGTTGT
AAGCAGGACCTAGGCCTGCTTGATTCTTTCCCACCTAAGTGAGGTTCTGGAGAGGGAGCCCCTTCCACCCTTCCAGACCCAGCAAAGGGCAAGA
TGGGAGCAAGAAGCAGAGGGTAGACGGGCAGAGCTAGGAAGGCAGCCCCCAAAAGAACTGTGGGGGAAATAAATCGGATGCCCTTGGGTGGCAG
CAACAATAAATCGTACAGCAGTGACAGCTTTGGCATGTGTTGTGTGTCTTTGTTGTGTTTGGGTGTGTTGTGTCTGGCTGTGTCCATTGTGTGT
CTCTCTAGGAGGCTGATCTGGAATGGGAGCGTCCTTATTGCGTAGCATATGGGCTTGAACGGTTCTCCGTGATTGTTGCCTTCTTCTAATGTAT
GCAGATGCTTTGAGGAGCCTGTTTCTATGGTTTCTTCCAGGGAAGTTGCTTTGTTTCTTTGCAGTGCTGTCCCAGAGACAACCTGTGGCCAGTC
CCCTACATATTCTTGAGCACCACAAGGTCCTATTATATAGAGCTGCTTTTGGAAGAGAATGTTTCTATTAATACCATTTGCAAAATGGAAAAAA
AAAACAGCATCCATGATTTAAAACATGCAGAGGATTTGTTTTAAGCAACCGAGGGCTCAAATTCAGTCATGAAATACCAGGCTGGCTTCCAGGA
TGGGGTGCTTGGTGAGCTGGTGAGAAAGCTCTCCCTAAGCATGGTGGGCCGATGGGTCTCAGGACCAGAATTCCAGGCAAATGTGAAGCAAATG
AGAAGATGGTTGAACCAAGTGTAGAGTGGCCTGAGGTCTGAGTTTGGAATGTTGCAATTTTACTCAGACGAAACAATGTTCTTTCTCACACACA
AGTTCTATGAGTAAATACTCCTAAGTAACACAGGTGGCAAGTCCATTTTCTTGAGGCTGGTCTCAAACTCCTGAACTCAAGTGGCCCTCTAAGC
CTCAAATTCTAGGCACCCGTATCACCACTCCCACTTAGCCTATTCGTTCTTAAAAAGAATTATTTATTCATTTTCTGTATATGAGTACCTGGTA
GCTGTCTTCAGACACACCAGAAGAGGGCATCAGATCCCATTACAGATGATTGTGAGCCACCATGTAGTTGCTGGGAGTTGAACTCAGGACCTCT
GAAAAGCAGTCAATGCTCTTAACTGCTGAGCCATCTCTCCAGCTCAACCCTATTTATTTTTAAGGATACCATTTGATAGTAGAACGCATAGCAC
CTGCTCACAGTTTAGTCACATGCTTGAAGAGAGAGGCCGGGGAATGCCATTCTTAGCTGCCAGCCATTTGCTAACCCAAGCTGAGGTGAGGGCA
GAGTTTTATTAGAGGAAGAAGATGGAGCATACTGGAAGAAATAAGCAGAAACATTACTCAACACTCAGACTCAGCCAAAGTGGGAGGAAAGAGC
CCTTGCTCTTGCTCTGGGCTGAGCCTTTGTCCCTTCTCACTAACCCAGCTTGTAGAGCCTGGAGCCCATTGCACTTAGATGGAACCGGAAGCTA
GGGTACTTACGGAATCAGTGGCCTCACAGCAGGCTGTGGGTCAGCTGCAACTCTCAGGGACATATCCAGCCACCTCCTGTGCAGCTGGGAACAT
TAGGACAATTTCTGGTCTGGCTGGGCATGGTAGCTCACATCCGTAGTCCCAGCATTCGGAAGACAGAGGCAGGCAGAGTTTATGAGTTTGAAGC
CAGCCTAGTTTATGTAGTTCCAGGCCAGCCAGGACTCATAGCGACACCCTATACTAGAGAAGGGGGTTGGGGGCGGAGGGAGGGAGTGAAAGCT
GCTTCTGGTCCTATCTCAGTACTCCAGGCCAACCAGGACTCAAAGTGACACCCAATAAATCAAGCAACCAAGCAAGCTTTTGGTCCTATCTCAG
TGCTCTCAGGAATGTCCGGTTTCCTTAGTGCCAAGAAGAACAAGAAGAATAGGACTGGCTTCCTTGAAGGCTGTCCAATTCTACCTCAGGGCAA
CTGCTTTCTGTGGACTACCAGCCAGCCACCTGCTCTGGAAGGTTCTGCTTCTCTGTTCAGGATGCCCGTGACGCCACCCAACCTGCAAGGCTGA
GTCAAAGGCCTTGTCTTCAGCATTCCTCTGCTGGGCCCTCTCCCCTCCTTCTGTTGATCTCTCCTCTGGAATGCCACACCAGCTAACAGGCTCA
CGGATATTTCTTTTGGCAGGTGGTTTTGTGGAAATTGTCCTGGTGGCAATGGAGTGGGCCTTTTATTTTTAAATAGGATCTCACTCTGTAGCCT
TGTCTGTGCAGGAATTCTGTAGACAAGTCAGGCCTTGAACTCATAGAGATCTGCCTGCCTCTGCCTCTCTTGAATGCTGATATTAAAAGTGTGA
GCCACCCTACCTAGCCCAAATCATTCATTGCTTTGACATCTGTTAGTTGCTTGTTATTTTACCAGGGCAGGGCCGAAGCACAGACTTTGTAGAG
AGGGACACATGGTACAGCTTTTAGCTTTAGAACCTAAAATAGCACCACCTCTGGGCAGCATGCTTGCAGCAGTATGCATCTGGGTGTCCAGCTT
CAGAAATGACAGCGCCCATATTTTGGGTGCCTGCCCTAGGACAGACAGGTTTGTTCATTTGTTTGTAGCGTTTGTTTTCTGCGGGGCATCAAAC
ACAGGGTTACATGCTAAGATGACAGTCTGACCGTGGCATCTGTACCTGCCCTGCAATAGGATTTTACAGTTATGTCTCCATTTAATCTTTATAG
TAACCCAATAGGACACACTACTAGTGTTCCTGTTTTTCCAAACAGAAATCTAAGGCCCAGAGATTTGCAAGTTCATACTAGACCACATATAGTA
AGTTACATTGTATTTCATGGCTCATTTAGAGACACGCAGACTCAATCAATACCACATGAGACTCTGAGCTCAGGTTTTCCAACCTCTGACCTTT
TCCCCTTTGATGGTGTGCCCAAGAAACACGGGGTTCTGGGCAATGAGCAACCCTAGATTGTAGATCTTCTAAGGGTGGGTTCGAAAACAACCTT
CCTTCCGTCAGAGAGAAAATTAAGGTCAAGAACACCCGTCCTCAGTGTCAATGGCCAGTCCTAGCTGCTGGACCCTTGAGCACCTGCTTCTTAG
GAGATGAACACGCCACCACTTACTCTTGACCTCTGATTGACAGCCCTCTGCCTCACCTGTCTCTGGCACAGCCTCTGTGCCAGGAACTGTTACA
GCAATTCTTAGAACCTTGGAGATCTCAGATCAGGAAGGACCCTGAGCAGAATGTCCCTCTTTTCCACCCCGTTTTCATCTCGTGCATATAATGT
CTGATTCTCTCAGTTTCGGGGATTCCTTAGTAGAACTAGAGAAATCACTGACAGAATCTTTCCCCTTCAGGGCCTTCCAGAACTTCTCAGATTT
CCGTAGCTGAAAGGATTCCCAGCCTCTTGCAGAGAATTGAGAAACTCACTATTTTTTTTTAACTCTTGTGGTTCCTTTCTCAAAAATTCAAGGC
TTGACTGGCCATATATATGTGTGCCTGTGTGTATGTGTGTACATATATATGTATATGCATGCATTCATATAAATTTCAGGATGACTCAAAGTTA
ATTTTCAACTAATACCCCTTAAAGATAATGCTAGTGACAGTCATAGCCGTAGCAACTGCTATGCTGGGTGCCATGGGGATTCCCCAACGTCATC
TCTCACACAGCGTTCCTTCCAGGTCTGTCCCCTGAGGCTCACGTGCTATATTGTATCTACTTGTCATTGAAAAACCACCCCTTTACCTGTTTTA
CTCAAGCGCATTTAAAAAGGAAACTATGTAACCACCCTAACTAGGAAGCCATGTGCTGCTCATAAGCACAAGTTCACTCTGGCCAAGTCCAGTG
CTTGCCACTGTAAGCCTGACTTTAGGGGGATGGAGGCAGGGGGATTGCAATCTCAAAACACAAGACAAACAAAACTTATATACAAATTTAACTT
CTTTTAAAAGTTCATCGAAGCCAGCCAGGATGGCTCATGACTTTAATCCCAAGCACTCAGGAGGCAGAGGCAGGCTGATTTTTGTGAGTTCAAG
GGCACCCTGGTCTACATAGAGAGTTCCAGGCCAGTCAGGACTATATAGTGAGATCCTGTCTAAAAACAAACAAACAAACAAATACAAAAACCAA
GAACAAAAAGTTTATGTGCCTTTCTACAATGACCAAGAGTCACAATTGTGCACACTAGGAAGACACAGTGCAATGTGGTACCAGAACAAGAAAA
AGTATACTTGGTGTGACCATGGATGGATGTCCTCCAACTTACAATAGTTCAACTTGTTCATTTTGAGGCAAGGTCTCACTCACTACATAGCCCT
AGCTGGCATCCTCCTGCCCCTGGCTCCAGAATGCTGGAATTAATGACACGTCAGGCCTCTGTTACGTTACAGTGGTGTAAAAGTCATTCACACT
CAACAGAAAGTATACTCTTGTTTCTGTTTTTGTTCTTGTTTCTGTTTTTGAGACAGGGTTTCTTTGTGTATCCCTGGCTGTCCTGGAACTCACT
TTGTAGACCAGGCTGGCCTCGAACTTGGAAATCCACTTGCCTCTGCCTCCCAAGTGCTGGATTAAAGGCATGCAACACCACTGCCCGGCGTGAA
GACAGTCTCTTGGTACCCAGGCTGTGAACTTGATATTTTGTTGAGCATGACCTTGAACTTCTTTAAGTATCATTGTTGAGGGGAGGCCTAGATG
TGTGTGTGTGACATCAGAGGACAACTTTGTGGGATCAGTTCTTTCATTATACTTTTACAAGTGTTCTAGAATTAACTAGGGTTAAAACTGCCTG
GCACGTGCCTTTTATCCACTGAGTTGTCTTGCTGCTCCAAACTTTGGGCTTCTGAACTTTTTGCCTCCATCATCTGAGTGCTGGCATTATAAGT
GAACAGGACTGGCCCAGAATGAGAACACACTCTTAATCCTAGCACGTGGGAGGCAGAGGCAGGTGGGTCTCCGTGAGTTCAAGGCCAGCCTGAA
CTACAGAGTGAGTTTGAGGACAACTGGGGCTATATAGAAAGATTCTGTCTCTCCCACCCACCCCAAAAAGGCACACACCACTGCTCCTCATTTT
ATTAGGCGATGAGATCAAACCCAGGGCTTCATGCACACGAAGCAAGCACTCTATCAACTGAGCCATAGCCTCCAAGTCAGCATTTAAACTTTTT
TTTTTTTTTTTTGGTTTTTCGAGACAGGGTTTCTCTGTGTAGCCCTGGCTGTCCTGGAACTCACTCTGTAGACCAGGCTGGCCTTGAACTCAGA
AATCTGCCTGCCTCTGCCTCCCGAGTGCTGGGATTAAAGGCGTGTGCCACCACGCCTGGCAGCATTTTAACTTTTTAAAAATTACACTTATAAT
ATATTGTGTGTATGGTACATGTGTGACAATCAGGACAACATTCAGAAGTCACTTCTTGCCTTCCACAATGTAGGTCCTGGGGGATTGAACTTGG
GCTTGGCGGCAAGCACCTTTACTTGCTGAGCCATTTTGCTACCTCTCCCTAGTTTTTTTTTTTTTTTAGATTTATTTATTTATTATTATAAATA
GGTATACTGTAGCTGTCTTCAGATGCACCAGAAGAGGGCATCAGATCTTATTACGGGTGGTTGTCATTGCTGGGATTTGAACTCAGGACCTCCG
GAAGAGCAGTCAGTGCTCTTACCCAGCCAGATTTCTTTCTTTCTTCTCGGTTTAAACTTTGCCTGTGTGGCAGTGCACACGAGTGCAGGTGCCG
AGTTGCTCCCCACTGGAGCTGGAGTTTAAAAGTGGCCACGAACTGCCTGATGTGGGTGCTGGAGTCTTCTGCAACCCAAGCTGGAGTCTTCTGC
AACCCAAGCTGGAGTCTTCTGCAACAGTAATATGTGCTCTCAACCACTGAGCACAGTTCCCTCAGTCCCTCAGCCAGCTTGTGCAGCCTGGACT
CACCTACTGAGGGATGCACTCACAGAGAGCTGCCCCCCCCCACATCAATCAGCAATCAAGAAAACAATCCACAGACACATCCACAGGCCAGTCT
GATCCAGACACTTCCTCGGCTGATGTTCGCTCTTCCCAGGTTTGTGTCAAGTTGAAAATAGAACTAACCAGAGCACAGGTTTTACAGTGTAGCC
CAGGTTGGCCAGGAATTCATGGTTCCCCTGCTTCAAGCTTCCCATGCTAAGGTTGCAGGTGTGTGATGACACCCCCAACTTTTGCATTTCCTAT
TTATATTTTCATCTTACAATGGACTCATTGAGGCATGAACCCATCATAAATTGGGGAGCACCTGAACCCAGTAAATGAAGTGTTGGGTGCTCCC
ATTGCCTCCCACTGTTCTCTATATTGACAGACAGCTTCTACCCCAAATCCATGCCAACAGTGTACACTTCTCTGTGGACACCTACAGCAGGTCG
TGTGATTTAACCTGATACTGTTAGGATATGAATTTAGTGCAGACTCTACAGTATAGGTCAAATTCTACAAGAACATAGAAGCCAAGTCAAAGCC
TAGGCCTTCCTTACTTCTGACCCACTGAGATAGGTCAGGGACTGCCACAAGAGGGCACAAAGGCAAACTGGCTCTGAGCCCTGTGCAAGGAAAG
CATGATGTGTGGTTGCCTTCACCCTGGCACTCTCCAAAAGCTTTGCCATAAGACATTTGATTATGCCGGGTGGTGTTGGTGCATGCCTTTAATC
CCAGCACTTGGGAGGCAGAGGCAGGCAGATTTCTGAGTTCGAGGCCAGTCTGGTCCACAGAGTGAGTTCCAGGATGGCCAGGGCTACACAGAAA
AACCCTGTTTTCATCAGGACAGAATGCTTACAGTAGAGAAGCCGCTTGTGATGTACCCAAGCTAAAATTAGACGTAGCGATACAAATGACAAAG
GCGGAAGAGAATTGTTATTGAGGAAAAACTTTGTCCCATAAAATATTCTTGGTAAACCCCCCAAATCAACTCAGATTTCCAGCAATCTGTCAGA
GCAAAACCCACAAAGCCTCTGTGTGTGTGTGTGTGTGTGTGTGTGTGTGTGCGCATGCATGTGTGTGCTTCTGTGCATGTGTGCATGCATGTGT
GTATTGGTGCTGGGGATTGAACCGAGGGTCTTGACACATGCTATAGTTATTCCACTGAGCTAAGCTCTCAATTCTTGTTTCCTCTTTTTTAATT
CTTTTTTTTTTTAATTTATTTATTATATGTAAGTACACTGTAGCTGTCTTCAGATGCACCAGAAGAGGGCATCAGATCTCATTATGGGTGGTTG
TGAGCCACCATGTGGTTGCTGGGATTTGAACTTCGGACCTTCGGAAGAGCAGTCGGGTGCTCTTACCCACTGAGCCATCTCACCAGCCCCTCTT
GTTTCCTCTTAATTGACCTAAAAATCACTGTTTGGAGGTAACACAGCAAGGCAATGAATTGCTGACTCTGAATCCCGTTAGCCTTGACATCGCT
AACTTGTGGCCTTGAGGACAGTTGCTTAGGTTCCTTTGCAAAAACTTAAAGGCTGCTTTTGCATTGGGATCGCGTGAGCCTGTAGATGGACCAT
AGACATGAGATAAGAAGCTCGGCTGGTCTCCTCTCCTGTTGTTCCTGCCCCTTTTGCTCTCTTACACTTGGAACTAAATTCTACTCTGCTTTGC
AGATAGACAAGCTGCTTATGGGAGAAATGTTTAGTCCAAGACTATAAGAGAACTTGGGACCGGGCAGTGGTGGTGCATACCTTTAATCCCAGCA
CTTGGCCTATTTCCCATTGAATCATTTCTTTTTTTTCACCCTTGGGCTTTCTCTGCAGTGCAGCAGCTAAAGACTGTACAGTCTTTCGGCCTTA
TCCTTAGGCTGAAACCAATCCTGAGGACAAAGCATTTATGGAAGAGGATGACATCAAGCCAGGTATGGTGGCCTGTGAGCTCTGCCTTGGTAAG
GTACAGATGGGAAGACCCAGAACTCAAAGCCAGTCTTGGATATATAGTAAAGTCCAAGGCCAGGCTGAGTTGTCTGAGATGGGAGTGGGTAGAG
GAGTGAATGGTGGCCATGGTGGTACATGCCTGCAATCTCAACTTTTAGGAGGCAGTGGCAGAAAGGTTGCAATATGTATAAGGCAAGCCAGGAG
GAGACAAGGCAACTATATCAAGAAATCAAAGACAGAAACTGAGCTGTAGCTCTAGTGGCAGAGTGTTTACCTGGTATGCTCAAGGCCTTGTACT
CTCTTCCCTGTATCCCAAAAAACAGAGATGTAGACGAGAGAGTTGGGAGTGCAGGGTCCTCTAGCTGTATAGTGAGTTCAAGGTCACCCTAGAA
ACCATGAAACCCTGTCTCAATAATAATAATAATGACATCAACAACAGAAGCAGCTCAGCTTCTGGGGTTAAGCTACGGATTCCAGGTTAGGATG
GTTGAGGTTGTGCAGAGCAAGGAAAAACTCCTTGACTTTCAAAATTCTGCCCTGACCACTGCTTTAGTTCAGTCTAGAAAACCAAATCTGTTGT
CTTCACTAAGCGAGCAGCTTTTTCACGTATTTGGCTTATACCAGCTCCCTTCTACTCCAGAGCTGAAGTCCCTTCAGGAAGCCTTGTCACACAG
CATGTCCTCATTTTGAAGGCCCCATGTCAGCAGAGCCTGCCTCCTGGTAGCGACAAGTCTGGCTGTATTCCCAGACTTCCCAGAAGCCTCTTCT
TGAGCTCCATCCTTTGCTGAAAGGGTCCACAAAACTCAGGGACACATCTGCTTCTGAGGAGTGGTTTAGGCAAAGGTGAGCACTCAGAATCAGC
CAGAGCACAGGGAAGATGAGGAGGTGAAGGCCCTCCTGTGTGACCCCTTCCCAGGCTCTCCATAGGGTCTCTACATCTCTTTTTCTGCTTCTAT
GATCAGTTCTACCTCAGAGATTAGCTCATTTGCCTTTGAAGATTAACTCACCTTCTGGTCCCTTCCACTCTCTGGACGTTGGTGGGGAGGTGAG
AACTAAGAGTTCAACCTTCTAGTGTAAGGGTCCATGATTTGCCGAAGAATGACACCCCAGACTCAAATAGTATGTAAACACAAAGAGTGTTTTA
TTCTGCAGAAGTCCAGCATGCTGGGACCTCCCATTACCAAGATAGAGAAACAACCAAGCGAGCTCGAAGGCCCAATTTAAAACACCTTAGGGGA
ATCGGGAGGCCGATGGGCTTTGTCTTACTCCGTCTCTAAGGTCATTCTGTCCCTGGGGCATGAGGGCTGGCTGGAAGCTGCTGACCTATTGTCC
TCGTTTCAGGCCAGGTGATGGGGCAGTTTCTGACTAGCTGCCTGAAGTTTGGGTTTTTAGGTCTAGTCTCTTTAACTGCCAATTTGAGGCCTGT
CTTGGAATCAGCCTAGCATTCTCACCAAGTCAGGATGGGTTCCACTGGTGGCCAGTCCCATTTTCCAGAAGCCACCCCAGTAGGATAAATGCAG
CTATGACTGAAAAGGCTAAGAAAAGAACACTAGCCTCTGTTCTTATTGCTGGGGAGAAAACTCAAGGGTTTCAGGGGCTCTGTGCCCGGACCTT
GGGAAAGAAGGCATGATTGCTTAGATTGAAAAGATCTGGCCGGGCGTGGTAGCACACGCCTTTAATCCCAGCATTGGGAGGCAGAGGCAGGCGA
ATTTCTGAGTTTGAGGTCAGCCTGGTCTACAAAGTGAGTTCTAAGAAAGCCAGGGCTACACAGAGAAACCCTGTCTTGAAAAACCAAAAAAAAA
AAAAAAATCTGAAGGAGCTTCAGGCAAATAAGACAGACTTTATTCAGTCTGGCTGAAGGTTGCTGGGAAGGCAGTCACTAGTTGGCATTCTTTC
AGCAAGCACCCAGGCACATGTACGAACTGCACACAGGCAGATACAACTACAGACAGATACTCACAGGCAGACAGTTGATGTTGGAGGAGAGGGA
CTATAGGTGTACTTGTGGGCTACTTCATTGGATTATTTTACTACCACCTTTCTCCTCCCCTGTTCCACCCTAATCCCTTCCAATCCCCCCAACA
CTAGGTAGATCTCTCTAGACTATTTCCTGCTGATTAAAGGCATCAAGTCCCTTGGGGCAAATCCAATCTTCATCCTCAGGATATCTCCAACCAG
CAATCGCAAAAGCAGCAAATGCAACTGCAGGAATCGGCAGAGGCAGAGCTGAAGCAGTGGGAGTAGCAGCAGCCACCTCCACAAGCCCTCTCAG
GGCTCTCACATTTATACCCTCTCAAGGGTTACCAGAATTCCAAATGTAAACTATCTGCTGCTG
MOUSE SEQUENCE—mRNA
CACAGGCAAGACTTTGGAGAGAGCAGACTAGGCATAATGGCCACCTTGCCACCAACAGCCAACCCTTCCCAGCAACCTCTAAACCTAGAGGATG
AAGATGGAATCCTGGATGAATATGATCAGTACAGCCTGGCCCATCCCTGTGTCGTCGGGGGAGGTCGGAAAGGTCGGACCAAGAGAGAAGCTGC
TGCCAATACCAACCGCCCTAGCCCTGGCGGGCATGAGAGGAAGCTGCTGACCAAGTTCCAGAACTCTGAAAGGAAAAAAGCCTGGCGCTGAGAC
GGAGCTGGAGATAAGGCCAGACCACAGACACCACACCCAGCAATGGATACAGGACATCACACCCAGCAATGGATACAGGACATCACACCCAGCA
ATGGATACAGGACCTTGGAGAAATTAGGAGTTGTAAGCAGGACCTAGGCCTGCTTGATTCTTTCCCACCTAAGTGAGGTTCTGGAGAGGGAGCC
CTTCCACCCTTCCAGACCCAGCAAAAGGGCAAGATGGGAGCAAGAAGCAGAGGGTAGACGGGCAGAGCTAGGAAGACAGCCCCCAAAAGAACTG
TGGGGGAAATAAATCGGATGCCCTTGGGTGGCAGCAACAATAAATCGTACAGCAGTGACAGCAAAAAAAAAAAAA
MOUSE SEQUENCE—CODING
ATGGCCACCTTGCCACCAACAGCCAACCCTTCCCAGCAACCTCTAAACCTAGAGGATGAAGATGGAATCCTGGATGAATATGATCAGTACAGCC
TGGCCCATCCCTGTGTCGTCGGGGGAGGTCGGAAAGGTCGGACCAAGAGAGAAGCTGCTGCCAATACCAACCGCCCTAGCCCTGGCGGGCATGA
GAGGAAGCTGCTGACCAAGTTCCAGAACTCTGAAAGGAAAAAAGCCTGGCGCTGA
HUMAN SEQUENCE—GENOMIC
ATGCCGCTGCACTCCAGCCTGGGGGACAGAGTGAGACTCCGTCTCAAAAAAAAAAGAAAAAGAAAAAGAAAATAATTTTTCTTGCAAAAATGGG
AGCACAGATGCACCGTTGCGGAGGGCGACAATGATGTCTGGGCTCGCTTCATTCTACATGGGTTGGGGCATGGGGTAGGGGGGCACGGCCCCTT
GGCCCGAGGTGGGAGGGTAGGGGACGGTGTCTGTGGCCCAGGGGTGGGTGGGGTGGGACAACGCATTGTTTTCAGGGCACTCCGTCTACACCCG
TTACACTCCGCAGCAAAGCCGTCCCAGGTCTCCACCCACCACCCCACCCATCAGGACACCAGGAAGACGCACTTGACCTGCAAGAGACTGTTTG
AATACATCTTCCCTAAGAGGATGGACTGATGCTGCAGGCTGGACAAACCGCCAAGCTGAGCTCCAAGGTCCCCCTGACTGAGCCCTGGGATCCT
CTTGACCTTCAGCAGAACTCCACCTTTGAAGTCCGGTACCCACCAGGCGGCTGGATGAGCGTGCCTCCATGCAGGGGCGCTCAGGCAGAAAGTC
AGCCAGAGCAGATGAAACCTGAATTGTTATCCCAACCAAAAATTACAGCACGATGTTGCCCTCCCTTGTTTTTTTGACATACACCTGGGTGTTA
AAGACCCCTCCATGTTGACCCCGTGGGGCATGGGCCAGACAGCCCAGTGGAGGAGACGACCAGGACCAGCCCTGGTGAACGTGAAAACCCAGAA
GTGACGGCATTGGACATCAGATATCAGTGACCCCAGCTGGAAACAGATTTGATATTTGAGAATTTTCACTGGAGCTTAAATATGATAAGTTTAG
GACGGTAGACATTTTTATGGGTTTTTTTGTAAGTATGATCTTGACTAATCAAACTGTCTATAAAAGAAATTGAATGGGCTGAATCAATGGCTAA
TATGGATACCTCAGGGGTTTTTGTGTGGTTTTTTTGTTTTGTTTTGTTTTGTTTTGACAGGGTCTTGCTTTGTCACCCAGGCTGGAGTGCAGTA
GCACAATCTTGGCTCGCTGCAACCTCTGCCTCCCCGGCTCAAGCAGATCCTCCCTCCTCAGCCTCCAAGTAACTGGGACTATGGGTACACACCA
CCACACACAGCTAATTTTTGCTTTTTGTGTGTGGTGGGTTTTTTTTTTCTTCTTTTTTTTTTTTTCAGACAGCTCCTTGCTCTGTCGCCCAGGC
TGCAGTGCAATGGCACGATCTCGGCTCACTGCAACCTCCACCTCCTGGGGTTCAAGTGATTCTCCTTCCTCAGCCTCTTGAGTAGCTGGGATTA
CAGGTGCCACCATGCTGGGCTAGTTTTTGTATTTTTAGTAGAGACAGGGGTTTCACCATGTTGCCCAGGCTGGTCTGGAACTCCTGACCTCAAG
TGATCTGCCTGCCTAAGCCTCCCAAAGTGTGGGGATTACAGGCATGAGCCACCGCTCCCGGTCTTAATTTTTGTATTTTTTTGTATAGACGAGG
TCTCGCCATGTGGCCCAGGCTGGTCTCAAACTCCTGGGCTCAACCAATGTGCCCTCCTCAGCCTCCCAAATTGCTGGGATTACAGCGTGAGCCA
CCACGCCTGGCCTCGTTTTTTTTTAACAGTGCTAAGAGGTAAGGAGGACTCCCTAGGAGCAGTTCGCAGTGGCAGGGAGGCATATTTTATTACT
GACATTGTTGAGAATTGATAGTGATAATAAAAGGCAGAATGATTTGGATCAGTTTAATTATTAAGTTCTTTATAGACAGCCTGGATCAGGGTAG
GGGACTGCTCCCACTGCCCTGTCCCAAAATGCTCCCTTAAAATTATTTCCAGATATTTAAGAGCTTTATGGCATGACCCAATTGCTATAAAAAT
TTTAACTTGAAGGAAAATCTATACTATAAAGTAATCGTTTCTTTAAAGATACATTTTAAGCAAGAGGGTTTGGAAAAATCTTTATGTCTACACC
TGGTAAGCACAAGCCATGTATATTCCGTTTTTCTTTCTTTCTTTCTTTCTTTGTTTTTGTAAAAAAAATTTGTTTTGAGCCAGGGTCTCACTCT
GTTGCTCAGGCTGGAGTGCAGGGGCGCAATCACCGCTCACTGCAGCCATGACCTCCCCAGGCTCAGGTGATCCTCTGGCGTCAGCCACCTGAGT
AGCTGGGACCACAGGCACGAGCCACCACACCTGGCTAACTTTTGTATTTTCTGTAGAGATGGGAATTTCGCCATGTTGCTCAGGCTGGTCTCAA
ACTCCTGGGGTCAAGCCATCCGCCTGCCTTGGCCTCCCAAAGTGCTGGGATTACAGCACCAGGTCCAGCCATTTCCTTTTTTTCTATGCAAGAG
TATTGATATATGTACTGAATCATCCCATACTTGCCAATGTATGTGATCACATACTTATCTTGCTGGGATCACAGGCAAGAGCCACTGCGCCTGG
CCCAAGTTTACTTTTTTTTTTTGGAAATGGAGTTTTGCTTTTGTTGCCCAGGCTGGAGTGTGATGGCGCCATCTCGGCTCACCGCAACCTCCGC
CTCCCGGGTTCAAGCGATTCTTCTGCCTCAGCCTCCAGAGTAACTGGGACTACAGGTGCCCACTACCACGCTGGCTAATTTTTTGTATTTTTAG
TAGAGACTGGGTTTCACCATGTTGGCCAGACTGGTCTCGAACTCCTGACTTCAGGTGATCCACCCGCCTCGGCCTCTCAAAGTGCTGGGATTAC
AGGCATGAGCCACCACGCCCGGCCCCATGTTTACTTTTATAGTCCCAGCACCAAGCAAGTGCCTGGCACATGGTTAGTACCCTAAACATTTGCA
GACTGGAGAATGCATGCCTCTCTGGTCTTTACTTTCCTCTTCTGTTAAATGGGGTCGGGTTTGCTGATCTCCCAGAAGACATCAGTAATAAAAT
TTATTATTTTAGATTTTTTGCTTCTTTCTTTTCTTCTTTCTTTCTTTCCTTCCTTTTTTTTTTTTTTTTTTTTTTTTTTTTGTGTGTGTGTGTG
TGTGACACAGAGTTTCGCTCTTGTTGCCCAGGCTGGAGTGCAATGGCGCAATTTCAGCTCACTGCAACCTCCACCTCCTGGGCTCAAGCAATTT
TCCTGCCTCAGCCTCCCAAGTAGCTGGGATTACAGCCATGTGCCACCAAGCCCGGCTAATTTTGTATTTTTAGTAGAGACGGGGTTTCACCATG
TTGGTCAAGCTGATCTCAAACCCCTGACCTCAGGTGATCCACATGCCTTGGCCTCCCAAAGTGCTGGCATTACAGGCATAAGCCACCGCGTCCA
GCCTTATTCTAGGTTTTTCTACTACTGTGTTTTATGTGGCTGTCTATTTTAGTTGTCAGGAGAAAATGTCACACCTCTATTATGGTGAAAACTG
TGTATTACTCATAGTTATCTATACATTTAATGCAAAAATATAATTTGTAGTGTACATGATGTTCTGCAACCTGTGTTTTTCATTAGGAATGTAT
TATGGACATCTTTCTCATCAGAGATAGAGATACATTCTCTCTCTAAAGAGTATTGAATGGCTACAGTGTTTCACAGAAGGAAACTGATCAGTCA
CCTACTGATGCACATTTAGGTTGTTTCCGATTTTTGGTTTTTGCTTTTTTTAGAGACAGGGTGTTGCTCTGTCGCCCAGGCTGGAGTGCAGAGG
CACCATCATGGCTCAAGCCATCCTCCTGCCTCATCCTCCTGAGTAGCTGGGACTACAAGTACACACCACCATGCCAGTCTTTTTTTTTTTTTTT
TTTTAAGATATGGGGTCTCGCTATGTTGCCCAGGCTGGGTTTCCTGTTTTTCACTACTACAAACAAATGAACTTCCTTGTACCTGCCTCTTTAC
TCACCTCTGGGAAGATTTCTGTAGGAGAGACTCCTGAAAGTGGGACTGCAATATCAGGGGGATATAAGCATTTCAAAATCTTATCAATGTTGCC
AAACCACCCATTAAAGACAGCATCTGGCTGGGCGCAGTGGCTCATGCCTGTAATCCCAACACTTTGGAAGGCTGAGGCAGGCGGATCACCTGAG
GTTAGGAGTTCGAAACCAGCCTGACCAACATGGTGAAACCCCGTCTCTACTAAAAATACAAAAATTAGCTGGGCATCATGGCATCTGCCTGTAA
TCCCAGCTACTAGGGAGACTGAGGCAGGAGAATCGCTTGAACCCGGGAAGCGGAGGTTGCAGCTAGCCGAGATTGCGCCACTGCACTCCAGCCT
GGGCAACAAGAGCAAAACTCCATCTCAAAGAAAAAAAAAAAAAGACAGCATCCACTGATATTTAAAACGATGATATCACACAGCCAAGAAAACT
TGCAAATGCAGCTGCCAGATTTGACATTTTTATCTGGGATGGGGTTGTGAGATCATATGGATATATTTTGTTTAACCTCATTTAGATTTCTTGT
GTCCTCTTGGAACACAGCGATGTCCAATCCACCTTAATTACGTATTCTGCCTTCAGCCTGCGAAGCTTCTGTTCCTTTGAGCTAGTGACTTCGT
ACATCCCTCAGGCTATCCAACAGAGAGGAAGTCTTGCTGTGTTGCCTAGGCTGGAGTGCAGTGGTGCGATCATGGCTCACTGTAGCCTCAACCT
CCTGGGCTCAAGTCATCCACCTGCCTCAGCCTCCTGAGTACTTGGAGCTACAGCCATGCACCACCACGCCTGGCTAACTTTTGGATTTTTTTTG
TAAAGATGAGATCTCGCTATGTTGCCCAGGCTGGTCTTGAGTTCCTGGGCTCAAGCAGTCCTCCTGCCTCGGCCTCCCAAAGTGCTGGGATTAC
AGGTGTCAGCCTTTGCATCTGGCCCACAAACAGCCATTCTTGTTCAAACTTTGCCTCCAGGCCTGGCGTGGTGACTCACGCCTGTAATTCCAGC
TATTTGGGAGGCCGAGGTAAGAGGACCACTTTAGCCAAGGAGTTCAAGACCAGTCTCGGCAACATAGGGAGACCTCATCTCTACAAAAAATAAA
ATTAGCTGGGCATGATGGTGCGTGCCTAAAGTCCCAACTACTTGGGAGGCTGAGGTCGGAGGATCCCCTGAGCCCTAGAGGTCGAGGCTACAGT
GAGCAAGGTTGCTCCACTGTATTCAGCCTGGACGACAGAGTGAGAACCTGTCTCAAAAATAAATAAATAGGCCGGGTGCGATGGCTCATGCCTG
TAATCCTAGCACTTTGGAAGCCGAGGCGGGTGGATGACTTGAGGTCAGGAGTTTGAAATCAGTCTGGCCAACATGATGAAACCCTGTCTCTACT
AAAAATACAAAAATTAGCTGGGTGTAGTGGTGCATGCCTGTGATTCCAGCTACTCAGGAGACTGAGACAGGAGAATCGCTTGAACCCGGGACAC
GGAGGTTGCAGTGAGCCGAGATCGTGCCACTGCACTCCAGCCTGGGTGACAGAGCAAGACTCTGTCTCAAAAAAAAGAGAAAAAAAGAAAAAAG
AAAAGAAAATTAAAATTAAATTAATTAATTAATTAAAAATAAAAGAATTAGAAAACAAGCTTCACCTCCTCCAGGAAGTGTGCCTTCATCTCCC
TATCTCCAGAGAGGTTAGGTGCCCTCCTGGGAGCTCCCAGCCCTCTGCGTTTACTCCAAGCTGATGGCGCTGGGTTGTCATAGGTGTGCCTAGC
AACGTCACGAAGGTGTAACTCTAAGCAGGAAGAAAATAAACTTCCTCGTGGCAGATTTTACATGCACCTGGAGGCTTGAGGATGGGAAAATGAA
ACCATCCCTGTAAACCTCATGCAATTAATCAGAGAACAGAGAGGAGAAATGGAAACAAGCCAGGCTTGCAGCACACCCAGCGCTGATCATGAGG
TCAGCTGCCCCCTGATCTGCTTCCTCTTAGTTGTCTGGTGCCTGCTGTTCTAGAATCACGTAGACCTGGTTACAAGATCATAGTTCCCCTTAAC
TGCACTAGAGATAGCAACTTGAACGTTATGAAACATTAAATGTTACTTTTGAGAAATTCCTTCAGGTCCTGCGTGCCGGTGAAGCGACTGACTC
CGCCAGCCTGAAGGACCCCACCAGGAGCTAACTCCCCAAAAATGTCTTTTTCACATCATGATGATTTCATTTCCCTTGCTCTGACCAATGACCC
CAATTTTCTAGCCTCTCACCCTCCATAATCCCCTTAAAAGCCCCATCCCAGAATTCCCTGGGGAGATGCATTCGCAGGTCTCCTCCTATCCCCT
TGCACAGTGCCCTGTGATCATGAAACTCTCTCTACTGCATCCCTGCTGTCCAGTGTAATAGGGCTGTGACAGGGCAGCAGGCATACAAATCTGC
TGTTCCTGTAACAAAAAGCACACCTCTACGTCTAGATGGAGCCACGTCTAGAGCCCTGCAGATCCATGCTAGAGGGGTTCCAGCAGGCTGGAGT
ACAATGGCGTGATCTCGGCTCACTGCAACCTCTGCCTCCTGGGTTCAAGAGATTCTTCCGCCTCAGCCTCCCGAGTATCTTGAATTACAGGTGC
ATGCCTCCACGCCCAGCTAATTTTTGTATTTTTAGTAAAGACGGAGTTTCACCATGTTGGCCAAGCTGGTCTTGAACTCCTGACCTGGTGATCC
ACCCGCCTCGGCCTCCCAAAGTGCTGGGATTACAGGCGTGAACTACCGCGCCTGGCCCTTTTTTTTGTTTTGTTTTGTTTTTTGAGATGGAGTC
TCGCTCTGTCGCCCAGGCTGGAGTGCAGTGGCGCGATCTGGGCTCACTGCAAGCTCCGCCTTCCGGGTTCATGCCATTCTCCAGCCTCAGCCTC
CCCAGCAGCTGGGACTACAGGTGCCCGCCACCACACCCGGCTAATTTTTGTATTTTTAGTAGAGACGGGGTTTCACCATGTTGGCCAGGCTGGT
CTCCAACTCCTGACCTCAGGTGACCCACCCAACCTCGTCGGCCTCCCAAAGTGCTGGGATTACAGGCGTGAGCCACAATGCCCTTCCTCCTTCT
CTCATAGGCTTCTTTCTTCCAGCACCTGTAAGATCAAGGCCAGGGAGACCTCCTTCCCTGGCTCCATCTGGGACAATCACAGTGTTTCTGTGGT
CCCCTGTGAGGCTACAATTCGCCTCTCAGTTCCTGCATTGCAGAGCAGGTGGAAGCTGAGCAAGCAACACTGATCTTAGCAGAGCCTCATGTAT
TCAACTAGACATTTACTGAGCTCAGACTGAGTGCCAGGCATTGAGGATACAATATGGCAGGCAAGATTCCTGCCCCGAGAATAATGATAATTAA
AATAGCAAGCATTTATTGCACACTGATTATCTTCTAGGCACTGCGCTAAGCATTTTTTTTTTTTTTTGAGACCGGGTCTCACTCTGTCGTCCAG
GCTGGAGTGCAGTGGCGCGATCTTAGCTCACCGCAACCTCTGCCTCCTGGGTTCAAGCGATTCTACTGCCTCAGCCTGCTGAGTAGCTGGGATT
ACAGGCACACACCACCACGCCTAGGTAATTTTTGTATTTTTAGTAGAGACGGGGTTTCACCATTTTAGCTAGGCTGGTCTCGAACTCCTGATCT
CGTGATCCACCCACCTCGGCCTCCCAAAGTGCTGGGATTACAGGTGTGAGCTACCGTGCCCGGCCCCAACCTTCTTTTCTTTTTCTTTTTCGAG
TGCAGTGGCACGATCATAGCTCACCGCAGCCTCAAACTCCTGACCTCAAGTGATCCTTCCACCTCAGCCTTTCAAAACAGTGGGATTGCAGGTG
TGCCTCACCTTATCCAGCCTAGGTGCCATTAATCCCACTGTATAGACGAGTAAACAAACATGCAGACTAAGCTTGTCTAAGAGCACACAGCTAA
CTAGGGGGCAGAACCAAGCTTTAGACCTGAGAAGCCCTGTTCCAGAGCCTACTCCTTTAACCATTAACTAAACTAAACCAACGGTTGCATGCCA
GTGGGGACAAATAATGGTAAAGCAAACAAATAAAAAAGATACTGGCCAGGTGGCTCACGCCTGTAATCCCAGCACTTTGGGAGGCCGAGGCGGG
CAGATCACCTGAGGTCAGGAGTTCGAGACCAGCCTGGCCAACGTGGTGAAACCCCGTCTCTACTAAAAATATAAAAATTAGCCGGGCCTGGTGG
CACAAGCCTGTAATCCCAGCTACTCAGGAGGCTGAGGCAGGAGAATCACTTGAACCCAGGAGGCGGAGGTTGCAGTGAGCCGAGATCATACCAC
TGCACTCCAGCCTGGTGACAGAGTGAGACTCTGTCTCAAAACAAAAACAAACAAACCAACAAAAAAAGGATGGAGTCTTGCTCTATTGCCCAAG
CTGGCCTTGAACTCCCAGCTTCAAGCAATCCTCCCAGCTTACCCTCCCAGAGCCCTGGAATTACAGGCATGAGCTACCGCACTAGGCCAATCTT
TGCTTAATTTAAGATTATTTTCCTCTTTCTCTGATCACCTGGTCTGTTATTATTGGCCCAATCCTGTTTTCATTCAAGTATCCTGTCTTCACTT
TCATTTCCTGGGTGTTAAGGAACTCATACATCATCCAAATCTGGGAAAACAAAGGCTAAATTCAATTCCGGCTCAGGCAAGGGAGAAACACAAG
CAGAAGTTCAGCTTTTTGTGTTTTTGTTTTTGTTTTCAGACACAGTCTCATTCTGTTGCTGAGGCTGGAGTGCAGTGGCTCGATCTCAGCTCAC
CACAACCTCTCCCTCCCAGGTTCAAGCGATTCTCCTGCCTCAGCCTCCTGAGTAGCTGGGATTACAGGCACTTGCCACCACACCAGGCTAATTT
TTGTATTTTTAGTAGAGATGGGATTTCGCCATGTTGGCCAGGCTGGTCTTGAACTCCTGACCTCAGGTGATCCACCCACTTTGTGGATCTCCCA
AAGTGCTGGGACTACAGGCATAAGCCACCACGCTCGGCCTCATTGTGTCTTCTGCCTCAGTACATCCATATTTTGAAATACATATTATTTTATT
ATAATTTGTTTTTGAGACAGGGTCTCCCTCTGTCACCGGGCACTGTAGCATGTGTCTTTCAAACATTATCTCATTTGATCCTCTCCCCAACTCT
AGGAAGAAGATACAATTATTATCATCCTTATTTTACAGAATTGGAAACCAAGGCACAGAGAGGTGAAGGGACTCGCCCAAGGTCACACGGAAAG
TGGTGGGTCAGGGGATTGAACTGAGGGCAACTGGCTCCAGACCCTGAACTCTCCAACCCTCTGCCACCCTCCTCCTAGGTGAGGGAGATTACAT
TACTGGGGACCCTGTTTCAGGATATTAAGGGGTTTTCCAAGATGTTTCTCATAGAAAAAAGAAAAAAAAGGGTGTGGCTCATGCCTGTAATCCC
AGCACTTTGGGAGGCCGAGGCAGGCAGATGACTTGAGGTCAGGAGTTCAAGATCAGCCTGTCCAACATGGTGAAACCCCATCTCTACTAAAAAT
ACAAAAATTAGCCAGGCATGGTGGCAGATGTCTATAATCCCACCTATTCGGGAGGCTGAGCCAGGAGAATTGCTTCAACCTGGGAGGAGGAGGT
TGCAGGTTGCAGTGAGCCAAGATCACACCACTGCACTCCAGCCGGGCGACAGAGACTCCATTTCAAAAAAAAAAAAGAAGACAAGCAAGATGTT
GAGTGTGACTGGGTGGAAGCCACTGACTTGTCCCTGGCACAAGTGTGCTGATATCCCTTCCCCCTCCTTCCTCACGTCCTGCATCTCAGCCAGA
ATTGGGGTGCCGGGGTTAGCTGTGTGGCCGTCATCCCAGGCCCAGGTGAAGCCCTCGATCAGGTGAGGCCATGATCCCAACGAGTGTTTTAGCC
CAGATCCTGCCTCTTCCCTGCTTTCTGCCGAGAAGCGGGACCTCCTCTATGGAAAATATCCAGCTGGGTGAGCCTGGGAGGGAGGAGGTGAGTT
GGGCTGGACTCAGGGACCGACTCTTCCCGTCTCATGACTGTGTTTACTGGGCTGGATTTTGGGAAGGGGCCAGATTGCATCAGACAGGGCCTGA
TGGGCTGGAGCCAGACTGTGGTCTGAGGAGGAGACACAGCCTTATAAGCTGAGGGAGTGGAGAGGCCCGGGGCCAGGAAAGCAGAGACAGACAA
AGCGTTAGGAGAAGAAGAGAGGCAGGGAAGACAAGCCAGGCACGATGGCCACCTTCCCACCAGCAAACCAGCGCCCCCCAGCAGCCCCCAGCCC
GGAGGACGAGGACTCCAGCCTGGATGAATCTGACCTCTATAGCCTGGCCATTCCTACCTCGCTAAGGCCCACTCAGCCATCTCCACGAATCCTT
CCTCCTCTCCCGAAATCAGGACCCACCCCTCTTGTTTCCTCTCATTTCCTTTCCTTTCCTCTTCGTTTCTTTCTTTCTTTTTTTTTGAGAGAGT
CACTCTGTCACCCAGGCTGGAGTGCAGTAGTGTGATCACAACAACTCAAACAACTCACCGCAGCCTCGAACTCCTGGGCTCAAGTGATCCTCCT
GCTTCAGCCTCCTGAGTGGCTGGAACTTCAGGTGTACACCACCTGCAGTGGTGAGATGGGGTCTCACTATGTTTCCCAGGCTGATCCCAAATTC
CTGGGCTCAAGCAATTCTCCTGCCTTGGCCTCCCAAAGTGCTGAGATTACAGGAGTGAGCCACCCTGCCCAGCCCACTCACCCTTTTCTAGCCA
ACCTGTTCCTTGGACCCTCACGTCACCCCTGTCTAATCCCTTATCCCAGGAGTGCTATGTTACTCAGCCTGGGACCTCACACACATCTGGGGTC
CCACATTCCACAGAGGGGAAGCAGCAGGCTTCTCCCTGCTCTTCCCATCCCCACAACCCTGAACCCCTGCCTCTCCTCTGACAGGGCCTCTCAT
CATGCCTATGCCCACTTCACCTCTGACTCCTGCCTTGGTTACAGGAGGTGGAGGCCGGAAAGGTCGCACCAAGAGAGAAGCTGCTGCCAACACC
AACCGCCCCAGCCCTGGCGGGCACGAGAGGAAACTGGTGACCAAGCTGCAGAATTCAGAGAGGAAGAAGCGAGGGGCACGGCGCTGAGAGACAG
CTGGAGGTAAGGAGTCGGGGGGCCCAGAGAGCTCAAGGTGGTGCTTCTGCCATGAAGGACAGGCCGGAAGGTGTGTGATTGGGTGGGGAGGAGG
GATCAGGCAACTGTTGTCTTGATGCAGAATAAAACGAGACATATGTTTGATTGTGAGTTTCCTAGTGGCCAGAGCAAAGTGGGAACACAGAACC
TTTCCAATTGAAGGGAAATTTGACTTACAGAGACAGAAATTGAGGATAGAGAGGGTGGGTCCTTCCCTGGAGTCACAAATCAAGTGAGTGGGAG
AGGCAGAATTAGAACCCAGATCTCTGTCCCTTTTACCACCTGCTTTTCCTCACCCCCCAGATGAGGCCAGACCATGGACACTACACCCAGCAAT
AGAGACGGGACTGCGGAGGAAGGAGGACCCAGGACAGGATCCAGGCCGGCTTGCCACACCCCCCACCCCTAGGACTTATTCCCGCTGACTGAGT
CTCTGAGGGGCTACCAGGAAAGCGCCTCCAACCCTAGCAAAAGTGCAAGATGGGGAGTGAGAGGCTGGGAATGGAGGGGCAGAGCCAGGAAGAT
CCCCCAGAAAAGAAAGCTACAGAAGAAACTGGGGCTCCTCCAGGGTGGCAGCAACAATAAATAGACACGCACGGCAGCCACAGCTTGGGTGTGT
GTTCATCCTTGTTCTTTGTGTGTTTTTGTTCGGGCATGTGTGTGCTTGCCTGTGCCTGCACATTCATGAGCCTGAGAGAGCATCTTTGATGTGT
ATTTGTGTTTGGTGTATGTATCTGGGGCAGGGAGTGTTCCTGCTCTTGCAGGGTCTACGCTGTGAATGCAGCTTTTGGTTTGTTTGCTTTTGCT
TGCATATATTTTAGTGCATACATTTCTGTGGGCTCCTACGTAGTGGAAAGGAATTTCTTCTGCTTTTTTGCGATACTGCCCATGAAACACGGCC
CTCCCCAGCACCTGTTTTTGTTGATTGTGTCCTGTTCATAGACGGGAACGCTACTTATGAGTGCCATCTAAAAGTCAGAGAAAACTGAGATTTA
AAATATTAAAAGCCAGGGCCGGGGGCAGTGGCTCACGCCTGTAATCCCAGCACTTTGGGAGGCCGAGGTGGGTGGATCACAAAGTCAGGAGTTC
AAGACCAGCCTGACCAACATGGTGAAACCCCATCTCCACTAAAAATACCAAAAATTAGCCAAGCATGGTGGCAGGTGCCTGTAATCCCAGCTGC
TCAGGAGGCTGAGGCAGTAGAATCGCTTGAACCCAGGAGGTAGAGGTTGCAGTGAGCCGACATCGTGCCATTGCACTCCAGCCTGGGTGACAGA
GGGAGACTCTGTCTCAAAACAAACAAACAAACAAAAACTAAAGTCTGGGAGCAGTGGCTCATGCCTGTAATCACAGCAGTTTAGGAGGCCGAAG
TGGGAGGATTACTTGAGCCTAGGAGTTTGAGACCAGCCTGAGCATCATAGTAAGACCCCATCTCTACAATTTTTTTTTTGAGACAGAGTCTCAC
TCTGTTGCTCAGGTTAGAGTGCAGTGGCACCATCTTGGCTCACTGCAACCTCTGCCTCCCGGGTTCAAGCAATTCTCGTGCCTACGCCCCCTGA
GTAGCTGGGATTACAGGTGAGCACCACCACGCCTGGCTAATTTTTGTTTTTTTGTTTTTTTGATACAGAGTCTCACTCTGTTGCTCAGGCTGGA
GTGCAGTGGCATGATCGCAGCTCACTGCAACCTCCGCCTCCTGGGTTCAAGCTATTCTCCTGCTTCAGCCTCCTGAGTAGCTGGGACTAAAGGC
ACCTGCCACCATGCCTGGCTAATTTTTGTATTTTTAGTAGAAACAGAGTTTCACCATGTTGGCCAGGATGGTCTCAATCTCTTGACCTCATGAT
CCACCCACCTTGGCCTCCCAAAGTGCTGGGATTACAGGCGTCAGCCACCACGCCTGGCCAATTTTTGTATTTTTAGTAGAGATGGGGTTTCATC
ATGTTGGCCAGGCTGGTCTCAAACTCCTGGCCTCAAATGATCTGCCCACCTCAGCCTTCCAGCCTTTGGGAGGCCAAGGAGGGAGGATCGCTTG
AGGCCAGGAGTTCGAGACCAGCCTAGGCAACATACCAAGGCCCTGTCTCTACAAAAATTTAAAAATTAGCAAAGCATGGTGGCTCATGCCTGTA
GTCCTAGTTGCTCAGAGGCTGAAGTTGGAGGATCCCTTGAACCCAGTTGGAGGCTACAGTAAGCCATGATGGTGCCACTGCACTGCAGCCTGAG
CAATAGAGTGAAACCATGTATTGAAAAAGAAAGAAAGAAAGAAAGAGAAAAAGAAGGAAGGAAGGAAAAGAAAGGAAGGAAGAAAGAAAAGAAA
GAGAGAAAGGAGAGAAAAAGAAGAAAGAGAGAGAAAGAAAGAAGAAAGAAAGAAAGAAAGAAAGAAAGAAAGAAAGAAAGAAAGAAAGGAAGGA
AAGAAAGAAAGAAAGAAAGAAAGAAAGAAAGAAAGAAAGAAAGAAAGAAAAGAGCGAGCCCCGGCACAGTGGCTCACACCTGTAATCCCAGCAC
TTTGGGAGGCCAAGGCAGGCGGATCACCTGAGGTCAGTAGTTCGAGTTCGAGGCAGAAGTTGTGGTGAGCCAAGATCACGCCATTGCACTCCAG
CCTGGGCAACAAGAGCAAAACTCCATCTCAAAAAAAGAAAAAGAGAGAGAAAGAAAGAGAGAAGGAGGGAAAGAAAGAGGAAGGAAAGGAGGGA
GGGAAGGAGGGAAGGAGGAAAGGAGGGAAGGAAATATTAAGGGATTTGCCCTAGCTCACTCAGGGCTCATATTCAGGGATCTTATTGTCTTCAG
ATATGAGGTGGGTCCCCAGGCCAAGGGGGCTGTGGGACAGGGTTCTGGGAAGTGAGATGGGGGAGGGAGATTCTTATTCAGTGCTATGTCTCAG
GACCAGAGTTGAAGAAAAATATGGGGCAAACAGGAAGTGTGGTTGGAGCTGAGATTATTTGTTGATTGAGATAAAGGTTTGGGTTGCTATTATG
AAGAATCAAAACAGGATACAAAGTTACTCCTCTCTTACTTAGCCCCAAGTCCTCCTGGGTTCAAATGGCAGCTCCATTGTGTTGAGGATTCCAG
CTATTTTTACTCACTGTTCTGCCACACCCAAAACATGGCTTCCACTTTGAGGCCCAAAATAGCTACTCCAATACCTGCCATCACGTTTGCATCT
CAGCCTGTGGAAAGAGTGTGGAAGAGACAGAGGAGGCCACACCTCTTCAATTTTAGGGCATAATCTAAAAGTAGAACACATCACATCTGCTCCG
AATGTGGCTACATGGCCACACCCAACTACGAGAGAGGTTGGGAAATGTAGCATTGAGCTGCTTAGCCACGTGGTAGCCAAAACTTGGGCAACAG
CAGGGTTTTATGATGAAAGGGAACCAGAGAGAGTGAATACACGAAGAAGCTAGCAAGAAACATTGCCCACATGCAGACTCAACAGATGTCGGGG
GAGAAAACTCTGAGATTGGGCCCCGGGCAGAGCCTGTTTCCTTTTCCCCTGTCCCTTTCACATAAGCCCCACACTGCAGAACTTGGAGTCAGTG
GTTCATGGGGACCCAAGGACATCCACAGAAGACCGAAGTACCTGATAGAGCAGGAACAGATGCACGTCAGACTACGACCAGCTTGAACCCTTGG
GGACCTGTATCCATCCCCTTCCCTTGAGTCCAGCCTAATCACAGCCACAGAATGAATCCATGACCCGGAAGGACATTTAAGGACCTCTGGCCCA
ACTCTCCCCACGCAACAGTGGGATTGCCTCTGCTTGTTTGAATACCACTATGGAGGGGGAACTTGCTACATTTAAAGGTTGCTGAATTTTTTTG
TTTTGTTTTGGGAGATGGAGTCTCTCTCTGTCGCCCAGGCTGGAGTGCAGTGGTGTGATCTCGACCCACTGCAACCTCCGCTTCCTGGGTTCAA
GCGATTTTCATGCCTCAGCCTCCCTAGTAGCTGGGACTACAGGTGTGGGCCACCACACCTGGCTAATTTTTGTATTTTTAGTAGAGACGGGGTT
TCACCATGTTGCCCAGGCTGGTCTCAAACTCCCGACCTCAGGTGATCTGCCTGCCTTGGCCTCCCAAAGTGTTGGGATTACAGGCTTGAGCCAC
ACCACCCGGCAAAGGTTGCCTAATTCTATCTCAGGGCAAGTCTGTTCATGTGGGCTGAAATCTGTCTCTCATACCTGCCCTTAAATAAGCTTCT
GTTTTTCTGGTCCTTTCTGTCTGAGATGACTTTAATTCCTCCCCCGACCCCATCTCATGAGGCTAAGCTTAGATGTTTCCTTCTCTTCCTCCCT
GATGGCTGCGTGCAGAAGCTCATACCTGCAATCCCAGCACTTTGGGAGGCCGAGGTGGGAGGATTGCTTGGGCCCAGGAGTTCTAGGCCAGCCT
AGCAACACAGCAAGACCCTCTCTCTACAAAAAATTAAAAATTAGCCAGGTGTGGTGGCTTGTGCCTGTGGGCCCACCTACTCAGGAGGCTGAGG
TGAGAGGATCTCTTGAGCCTGGGAGGTGGAGGCTGCAGTGAGCCATGATCTCACTCCAGCCTGGGCGTCAGAGTGAGACCTGTCTCAAAAAAAC
AAAAACCAAAGTTTTCCTTAACTTGCAATCCTCCACCCATCTCCTGACCTCCACCCCAGGCCGACCCTCCTCTGGGATCACACGGAAGCCTGGG
CCCAGAATCTTACAGTCTACCTTGGAAATCCTTCAGATTTTGCCTCCAACTCCTCTTTCCATACAAGTTACATATTAACTCAATCAATGCTTAT
TGGATGCCTTCTGAGTTACCAGGATGCTGCTGGAGGCCTGTGCTGCTGAGAGGGACAAATAGAATCCTGATATGACTGGAACCAGGAATAGGGC
CACCATCAGCTCTAGAACCCAGGGAGCAAATGGCAGGAGCAACACCAGGAAGTCGGCCCTCAGCCAAGCACAGACCTGGTGTGTTGGCTTCAGA
AAATCCTGGAAACATCTCTGCTGTGTTTGGAGCTCCTATTCTGTGAGAAGTGTTTTATAGGCTTTGCTCCATTTAATCCTTACGATAACCCAAT
AGGACATACTATTAATGATCCCCTTTTTTTCAGACCGGAAAACTGAGGTTCGGAGACATTAAGTAATTTGCCTAAAGTCACACAGCCACATCCT
GTAGGACTTGGAATTATGAACTCAGGTCTATCTGAATTTTAAAGCCTGGATTTTTCCCCCTTTGCTACATGCCTGGGAAGAACCATGTATTGAC
AATGAAGAATCCTAAACTCTCGTTAATTTCTGGTCAATTACTCCTCTGGGCCTGGGTTTCCTCACCTGTCAAATGGTGAGACTGGCCTCTCTTC
TGTCAAAAGAGAAAATGAAGGCTGGGAAGCACTGTGTCCCTAGCTGGAATGGCCACTCCCTGTCCTGGGTCCTTTGACACCAGCCAGGAGTCCT
CACACACTGCCCCTCCCTCCTGGCCTCTGATTGCCCTTTCCTGGCCTCGCCCTCGCCTGGCACAGCCTCTGCCTGAGAGGCCTTTAAGTGGCTG
TTCTGGGAATTAAGGCAATTCTTGGAATTGCAGAATTTTTGGATCCGAAAGGATCCTACCAAGACTCTTCCTCTTCTGTCTCCATCCCCTTTCC
TAAACCCTTTCTTTTTCTTCTGGTTGCATGTCTGTTTCCCCATTTTGGGGGGATCCCCCTGAGAACTAGAGAAATCACTGACCTCTGTCCTACC
CCAGGGGCTTACAGAATTGGCCCTGTCAGGCTTCTGTAACTCAAAGGACTCCCGACTCCTTGCAGAGAACTGAGAAACTCACCATGGGGTTGTC
TGTCTGTTCTGCAAGCAACCTGATTCTCTTCCACCTTCCCCTTTCTCAAAGGCCAGCTCACAGATACAGGAGTACATTTTTAAATTTCAGAATC
ACCCTCAGTTTTCTCCTTTTTGAGACAGGGTTTCAGTCTATCACCCAGGCTGGGGTGGAGTGGTGCAATCACTGCAGCCTTGACCTCCTGGGCT
AATTTTTAAATTTTTCGTAGAGATGAAGTCTCGCTGCTATGATGCCAAGGCTGGTTTCAAACTCCTGTCCTCAAGTGATCCTCCTGCCTCGGCC
TTCCAAAAGTGGTGGGATTACAGGCATGAGCCACCGTGCCCAGCCCCAAAATTGATTTTTTAACTAAAATCCCCTAAAGTTAATGCTAATGACA
GTAATAGTTCCAGCTACCACTATTGATAATCTACCTTAGGGTTTTCCCAACTTCATCTTTCCAATAGCACTTTTGTGATTGATTTCTGCTGTCT
ATACATTCAAACTATACTATATATATTATGCTATATTTACTTAGCATTTAAAAACTACCTCCTGCCAGTCACAGTGGCTCACGCCTGTAATCCC
AGCACTTTGGGAGGCCAAGGCGGGCAGATCACCTGAGATCAGGAGTTCGAAACCAGCCTGGCCAACGTGGGGAAACCCCATCTCTACTAAAAAT
ACAAAAATCAGCCAGGCGCAGTGGTGGGTACCTGTAATCCCAGCTACTCAGGAGGCTGAGGCAGGAGAATTGCTTAAACTCGGGAGGCAGAGGT
TGCAGTGAGCCAAGATCGTTCCGTTGCACTCCAGCCTGGGCAACAGAGCAAGACTCTGTCTCAAAAAAAGAAAAAAAAACTACCTCCTTATCCT
TTTCACTCAAATATACTTAAAAAGGAAGCTACAGCATATAACCACCAACAATGGAAAACCATGGTCATATGTCATAATTAGAAATTAATTGTAG
GCCAGACACAGCAGTTCACGCCTGTAATCCCAGCACTTTGGGAGGCTGAGGAGGGTGGATTATCTGAGGTCAGGAGTTTCACACCAGCCTGACC
AATATGGTGAAACACCATCACTATCAAAAACACAAAAAAATTTAGCTGGACATGGTGGCAGGCAGCTGTAATCCCAGGTACTAGGGAGGCTGAG
ACAGAAGAATCGCTTGAACCTGGGAGGCAGAGGTTGCAGTGAGCGGAGATCGTGCTACTGCACTCCAGCCTGGGCAACAGAGCAAGACTCTATC
TCAAAAATAAATAAATAAATAAAATAAATTAACTGTAAAGGCTGAGCATGGTGGCTCATGCCTGTAATCCCAGCACATTTGGAAGCTGAGGTGG
GTAGATTACTTGAGGTCAGGAGTTTGAGACCAGCATGGTGAAACCCCATCTCTACTAAAAATACAAAAATTAGCTGGGTGTGATGGCACACCCC
TGTAATCCCAGCTACTCAGGAGGCTGAGGCAGGAGAATTTCCTGAACCTGGGAGGCAGAGGTTGCAGTGAGCCAAGATCGAACCATTGCACTCC
AGCCTGGGTGACAAGAGCGGAACTCCATCTCAAAAAAAAAAAAAAAAAAAAAAGGAAAGGAAAAGAAATTAACTGTAAAAAGAAATACCGAAGT
TAGCCAGTGTGGTGGTGTGTGCCTGTAGTCCTAGCTACTCAGGAGGCTTAGGTGGGAGGATCGTTTGAGCCCAGGAGTTTGAGGCTGCAGTGAG
CTGTGATTGCACCACTGCACTGTAGCCTGAACAACAGAGCAAGACCCTGTCTCAAAAAAAAAAAAAAAAAGGAACACTTAACTAAAACTTTTTT
TAAAGTTCATCCAGCCAGGCGCGGTGGCTCATGCCTGTAATCTCAGCACTTTGGGAGGCTGAGGCAGGCGGATCACCTGAGTTCGGGAGTTTGA
GACCAGCCTGACCAACATGGAGAACCCTTGTCCCTACTAAAAATACAAAATTAGCTGGGCGTGGTGGTGCATGCCTGTAATCCCAGCTACTTGG
GAGGCTGAGGCAGGAGAATCTCTTGAACCCAGGAGGCAGAGGTTGCAGTGAGCTGACTCCAGCCTGGGCGACTAGAGGGAAACTGCATCTCAAA
AAAAAAAAAAAAAAAGGCCAGGCACGGCAGCTCACACCTGTAATCCCAGCACTTTGGGAGGTCGAGGTGGGTGGATCACCTGAGGTCACGCGTT
CAAGACCAGCCTGGATAACATGGTGAATTCCCTTCTCTACTAAAAATACAAGAATTAGGTGGGCATTGTGGCACTTGCCTGCAGCCTGTAGTCT
CAGCTACTTGGAAGGCTGAGGCAGGAGAATCGCTTGAACCTGGGAGGCGGAGGTTGCAGTGAGCTGAGATAGCCCCACTGCACTCTAGCCTGGT
GACAGAGCAAGACTCCGTCTCAAAAAAAAAGAAGAAGAAGGGTAGGGCGTGGTGGCTCACACCTGTAATCCCAGCACTTTGGGAGGCCGAGGTG
GGTAGATCACCTGAGGTCAGGAGTTCGAGACCAGCCTGGCCAACATGGTTAAACCCCGTCTCTACTAAAAATACAAAACAAAACAAAAATTAGT
TGAGTGTGGTGGCACGAGCCTGTAATCCCAGCTACTTGGAAGGCTGAGGCAGAAGAATGGCTTGAACCCAGGAGACAGAGGTTGCAGTGAGCCG
AGATCGCACCACTGCACTCCAGTTGGGTGACAGAGCTGAGACTCTGTCTCAAAAAAAAAAAAAAAGAAGAAGAAGAAGAAAAAAAATTATGGGA
GATCATTGTTTTGGGTTGAGCTGCTGCACTTTAGGCCCCAACAGATAAGACCAAACCAAAATGTAGTTACTCATGCTAAATGTCACATAATCAA
ACTGAAACTTTAAGGAAGGAGATAGATCCCAAACAGACCAGTTTTTCCTGAAAACAGGAGATTCCAGTCAACCTGAGTCAGTGTAATATTGGTC
CCCTTTGCTTTAACTCTCACAAAAAAGTCACCTGAAGGAACCTCATGTTAACCAATCAGCTTTTTTCCTATTGTTCTGTTTCCTTGGCCAGTGA
GAGATCTTGATTCTGTTTTGTAGAATGAAAGCTGTACCAATTCATGAATTGCAAATAAAAGCCAATTAGATCTATATCCAAAATTGTTGTCATT
TTGTCTTTTGACACGGTGAAAGGTGAATAGGAGAAACTCCACCTTGGTTTGTATATAAAAATATTATCCAGGATGGTGGCTCACGCCTGTAATC
CCAGCACTTCGGGAGGCCAAGGCGGGCAGATCACCTGAGGTCAGGAGTTCAAGACCAGCTTCGTCAACATAGCGAAACCCCGTCTCTACTAAAA
ATACAAAAATTAGCCAGGCTTGGTGGCTCGTGCCTATAATCACAGCTACTAGGGAGGCTGATGCACAAGAATTGCTTGAACCCAGGAGGCAAAG
GTTGCACTGAGCCGAGAACACTCCACTGCACTCCACTGAGCCACTGCACTCCAGCCTGGGAGTGAGACTGCCTAAAAAAAAAAAAAATCTAGCC
ACAGCCATTATTATCACACTGGATATTTACCAAAGCCTGCACTAGGGAAAAACACCTGGGTCACGGGTTCACTGCTGGTTGGAGGCTGAGCCAA
GAGCAAACTAAGTGTGCTGATACCCATGCCCCTGATTTCTAGGGAGTTCTGTGGCCTTCAGGGAGTCCCAGGGGAGCAAGATTAGAGCACCCAG
TCCCTGAGTGCCCTGCTATCAGCGGCCTTGACAGAGGCGATCTCTCCTTGGTGGCTGGAATCAGCATCCAGGGCTCTGTCAACTATTAGAGAAA
CATTTTATTCTTTTTGTAAAACAAAAGTTCTCTTTTCTTTTCTGATTACAAAAGAAGTTATTGCTGTGGAATCGCAAGATAATCCCCATCCTCT
GGTCTGTCTGTCCTTTGTTCCCATGACTGAACTCCAACTTTTGCAAAACACTTACTTTTATTTTATGTTTTATTCTAGAGACAGGCTCTTGCTC
TGCTGCCCAGGCTGGAGTGCAGTGGTGTAATCTCGGCTCACTGAAGCTTTGACCTCCAGGGTCTCAAGTGATCCTCCCACCTCAGCCTCCTGAG
TAGCTGGGACTACAGGCACACGTCACCACACCCTAATTTTTGTATATACACACACACACACACATATATACGTATATATATGTGTGCGTATATG
TGTGTGTGTGTATATATATATATAGTGTGTGTGTGTGTGTGTGTGTCTGTGTGTGTAGAGATGGGTAGGGGGTGGGTCTCACTATGTTGCCAAG
GCTGGTCTTGAACTCCTGGGCTCAAGTGATCCTCCTGCCCTGGCCTTCCAAAGTGCTGGGATTACAGGTGTGAGCCACCATGACTGCCCTGCAA
AAACATTTAAATTATAAAATTAATGTTTCTTTGTCAGCACTTCATGGAATCAGTACTTCATGGAATTCTAATATCAGCTTTGTATCCTGTTCTC
TTTTCTGGAAACTAGTTCGGAAAGTTTCATTTCTTTTTTTTTTTTAGATGGAGTCTTGCTCTGTTGCCCAGGATGGAGTGCAGTGGCGTGATCT
CAGCTCACTGCAACCCCTGTCTCCCAGGTTCAAGCAATTCTCCTGCCTCAGCCTCCTGAGTATCTGAAATTAAAGGTGTGTGCCACCATACCCA
GCTAAATTTTGTATTTTTAGTAGAGACGGGGTTTTACCATGTTGGCCAGGCTGGTCTCGAACCCCTGACCTCAGGTGATCTGCCTGCCTCAGCC
TCCCAAAGTGGGATTACAGGCATGAGCCACCGCGCCTGGCCAGAAAGGTTCATTTCTTTAACTGTTTGGGGAAATATCATTTATTCCCTCGACA
AATACTTTCTGAGTACCTACCATATGCCAGGGAATAGTCTAAAACT
HUMAN SEQUENCE—mRNA
CAGCCTTATAAGCTGAGGAGGTGGAGAGGCCCGGGGCCAGGAAAGCAGAGACAGACAAAGCGTTAGGAGAAGAAGAGAGGCAGGGAAGACAAGC
CAGGCACGATGGCCACCTTCCCACCAGCAACCAGCGCCCCCCAGCAGCCCCCAGGCCCGGAGGACGAGGACTCCAGCCTGGATGAATCTGACCT
CTATAGCCTGGCCCATTCCTACCTCGGAGGTGGAGGCCGGAAAGGTCGCACCAAGAGAGAAGCTGCTGCCAACACCAACCGCCCCAGCCCTGGC
GGGCACGAGAGGAAACTGGTGACCAAGCTGCAGAATTCAGAGAGGAAGAAGCGAGGGGCACGGCGCTGAGACAGAGCTGGAGATGAGGCCAGAC
CATGGACACTACACCCAGCAATAGAGACGGGACTGCGGAGGAAGGAGGACCCAGGACAGGATCCAGGCCGGCTTGCCACACCCCCCACCCCTAG
GACTTATTCCCGCTGACTGAGTCTCTGAGGGGCTACCAGGAAAGCGCCTCCAACCCTAGCAAAAGTGCAAGATGGGGAGTGAGAGGCTGGGAAT
GGAGGGGCAGAGCCAGGAAGATCCCCCAGAAAAGAAAGCTACAGAAGAAACTGGGGCTCCTCCAGGGTGGCAGCAACAATAAATAGACACGCAC
GGCAGCCACAGCTTGGGTGTGTGTTCATCCTTGTTAAAAAAAAAAAAAAAAAAAAAAAAAA
HUMAN SEQUENCE—CODING
ATGGCCACCTTCCCACCAGCAACCAGCGCCCCCCAGCAGCCCCCAGGCCCGGAGGACGAGGACTCCAGCCTGGATGAATCTGACCTCTATAGCC
TGGCCCATTCCTACCTCGGAGGTGGAGGCCGGAAAGGTCGCACCAAGAGAGAAGCTGCTGCCAACACCAACCGCCCCAGCCCTGGCGGGCACGA
GAGGAAACTGGTGACCAAGCTGCAGAATTCAGAGAGGAAGAAGCGAGGGGCACGGCGCTGA

TABLE 36
MOUSE NOMENCLATURE
ICSGNM Zfhx1b
Celera mCG8151
HUMAN NOMENCLATURE
HGNC ZFHX1B
Calera hCG19701
MOUSE SEQUENCE—GENOMIC
CATAGCTTTGGCCAAACAGCATAGCTTTGGTCCTGAGGGTAGACTAAAGCAGTCCTAAACCTTTGTGTCTTTCCCAGCTTGCTGAACAACCCAA
AGTAGAAACAGAGCGCCTACTCAGAGTACTAGCAGTTTTAAAAGTTTAAACTCCACACAAGTTCTTTTACAAAAGAGTCCACCCTTTTATGTAC
CTTTAAAAACATTTAGCCACTTTATTCCTTTGTGTCTTTGAAGAGGAAAAAAAATTAATAAAGTTGTAGGGTCCTTTTGAATTTAGAAAACGTT
GGCAATGTGGGAAAGAAAGGCATTCAGCATTTCAAAGTTCTGGTGAGGAAGGAAAAAAAGATTCTGAATAAAACAATTTTATGTGATTTTGCAG
GTGGTTTGAGCTATCATTTTTATATGTACGTTTGTACCACTGCTCATTGGAATGTTCATATAATACTGACCTAATTATGCATAGAAAAGATAAG
CCATTTACTTGATTCATTTCTTCTTTTTAAATCAAGAAAGGAACAAAAAGGCTCCTATTTTTCTGAAAGAAATTGTTCGTTTTATTGCAGAAAA
GTAGCAGCTTGTGCAGAGCTAGAACTGTAACAATTTTATATTCCAAAGGTTTTGTGTGTATTCGGTTAGTTACAGTAATTATACCCAATAACTG
ACAGCCAAAATCACGTAGGAGAAAGCAAAGGCTGTTTTCTCACACGGGCCCTGCTGCAGAACATCTGCTGAGAGATTTGCATATTAATTAACCC
ATATTAACTCTAATTATTCTGGGAAATTATCAAATAAATTGAATTTTCTAATTTTAACCTTTCTTTTACTTGACGCGTGTCGAGGCTGAGAGGA
CGCACCAGGCCGTCTCACCTGGGCAATGAGCAAAGGCTCTTTTCTCCCCCTTTGCTCCTTTAGGCCTGGGGCAGCCTGGGGAGCCTTTCTGTGC
CTTGTGCTTTTTTGGCTTCTGTGCTTGGTAGGGTCTCCGTGCATTTGTTGAGAGAGAGAACATGTGTGCCTGTGAGCACAGATGGTGGATTTAT
ACAGGGGTGAGGAAAGGTGCTACCTCAGAACACTTGAGCAAACCTTAAGTTGTTTTTGTGCGTTGTTTTGTTTTGGGTTTTTTTTTCTTCTTTC
TTTCTTTCTTTCTTTCTTTCTTTCTTTCTTTCTTTCTTCCTTTCTTTCTTTCTTTCTTTTCCTCCCAGCCTGTTGTTTAGGCCTGAGCAAATAA
ATGGGAGTTTAATTCAATTAGAGCCTGGCTTTAGTCAGTGCCTCATGACAGAAATTATTCAATATTTTTCCCCTCACATTGTTTGGTTTACACA
TCAGTGACTCCCTTCCTTTTGTACTCTGTTTAGAGTGGCCATAATGTGTTGAGCTTTTGTGTCTGAGATGTCACTGTCCCTTTCACCGTTGTCT
GGTTCTGTCAATTGTGAGGTGCAATGACGCTATTTTGCAAACAGGCCTCTGAGAAGGGTACTGCTATGTTAGCCGAGGTGGCTTCACAGTAAAT
TATTTTATTAGTCTCACAGACCTGAACCTAATCCTCTGGGTAGGGAGATGAAGTTTGGAAGTCATCCTTGGGTATTAGACGTAACATAAAACAG
AGATTTCTAGGACAAATCCTGTAGGGGCGGCATGAGGAGCTCAACTCACTGGCATGATCTTGTTTTAGGTGTTTCTTTGGGGTAAGAGACAAGA
TTTACCAAGATTAAGATTCTCTGCATGTGATTAAGTTTGTAATGACCCATGGTTTTAAGCCATGGTGATCAGGCGAAAACTACTGATTTAATCT
GTATGAGGAACAAAGTCTTCCACAACTGCTTTCTGTTGAGCCACACCTCTCCAAAGACAGATGACTCTGTATTCCCCCAATGATTTCACTGCCC
CTACCCACCAAGATTTATTAAGGTGATCCACACCCCTGGCTGCCTTACAGGGGATTATTTTCTTCTGGAAAAGCTGTCCTTCCTATTATTCCAC
AGCTGGGTTAGACACACCGCTGCAGCCTCACAGAACATTACTTAGACCCAAGAATTATTATTGACACCAGTACACTGCAGCAACCAAGAAACTC
ACCAAATAGGTCTGTCCGACTGACACATACTTGAGAACAGTTTGTTTTTTTTTTTTTTTTAATAGATTGACTAGAACTGGCACAGAAAATCTTT
TGCTGGGAAAACATTTCTTTAATATGTTTTTACTTCACGTGAGGATATAGATTCAAGTTACTCTGTGTGTGTGTGTGTGTGTGTGTGTGTGTGT
GTGATTAATTTGGTGGGATGAAATGGCTAAGTGTATATCAGTATCTGTATCTTAGAAAACTTCGCTATCACAGATGAGGTTCACGGGTGGGGCA
AATCTGTAACAATTGGAGAAAAATATTAATAGTTTTTCTAGTCTGTAATTTTTTTTTTTACAAAACATTCCAAAGTATCCTGAAAGTTACTGAC
CTTACACCTTTTATGGACTTTGAAAGTTGTGTAACCTTTTCAGGGGAAGGTGACAGACACAATATCTAATTACACAAGGACATACTTCCCATCT
TGTGATACTTGCTTTATTCAAGGTTTTATTCCCAAGGCCTTATGACTATAAAACTTAAAGCTAGACAAGGTCTGCCCTCATTTCTGATAAAAGA
AAAACCCCAAAACAATTCTGATGGCCCAGAGCATATTATTTTCTACTTACAGGTCCTAAATAAATTACAGTTCTTTTCACATGCCTCAGGAATG
GCAATGAACATTCTTTTCATGTTGCCTCTTATGACTTTGCTCGGGCCGGTTTACATATAGCAACTCATTGCATGATCATGAAGCCCAATAGTTA
GAGAATCTTCTAAACTCCTTTTGCTCTTTGGACATGCTGTTGGTAGACATAAATTCATTTGGTATTAATAAGAGTAATAAGGCTCTGAAGAACT
GATGACGAATAGATACACAATGTATATAAAATGGATTTGTAGAGCAATTGTTTTGAGGTTTTCCAGAAACATGCTATCAGGACAGGCTCGCTGA
TACAGGTACTTTGGGACACATTGTTTAGCCAGTGATGGGATTAGCTATGTGGTCTATCTTTGGACTGCAGATATAGACAGGGAGTGAAGCTGAA
AAACATTCTGAAGGATGGAAATAAAAACTACAAAGACTTGCTGGATAAAAACAGAATGGAGCATGCTTGAACCAACAGTACAATGACTACTTTC
CTTTGGCAGGGGTTGAGCAAGGTGATCACAATCATTGAGTCATAGATATACAGGAAAACCAAACATATTTACTTTAAAAAAGAAAAGAAAAGAA
AAGAAACAGATGTCAAAGCTTTGGGAAATCATGGTTTTTCACTTAGAACACGGTGACCTGGGAATAAAGTGTCTTGTATTAAAAGATAACTGAC
TTCACTATTACACATTCTTTGACTAATATCTGTTTTATGGAGATGGCAAAGGGAGTCTTGCTTTTGTTCACATCTGCCATCACCTTTTAATGTT
TCCTATGTTAAGCAAGAAGTTTAGAGGGGAAATCAGATACCTAAGATGCCACTACCATGTGCAAGTTTAATCAGAACCTCTATAACTTTATGGA
AATTTTCTTTTTAACAATCAGTATCATACTAAAATTTGGAAACAGTAGTGATTCCAGTTGGATGAGAAGCTGCCAGTCTCCCTAAGATGCTCCT
GTAACAGGGTGGGGGGACCCTCAGAATGTTCCCGCCCACAGAATTGTGGCAAGGCAGAAATTCCTCTCTTGTTTCATTTAATATGCATATTTTT
ACATAGCTAATCTGAAATGGAAACATTTTATCTCTACATTCAAAACTGCTTTTGTTTTATCAGCTGACATTTTACAACTCATTAATTTATAATG
CAGGTTTAATGCCTTTGAATGTTCCTGGGATAAAAATTAAAGTGACCAAGCTAGTTTATAAAGGCTCACTATAGTACGTTGGTAATAATTCTTT
TATAACAGTGTGTTGTACTTAATAGTGCATTCCATTTGGATGTTTTCAGTTGGCATAGTCTCTCTCTAGTATTAATTCATATTTTCACATCTGG
GTGATAGATAAATCTCCTCCTGTATTAAAATTTAGAAGTTTAAGTTCATGCACTTTGATGACATATGCTCAAATTTGGAAGAAAATCTCTGTTT
GAATCTGTCTTTGCCTTGTAATGCTCACTAGACCAGATTATAAAATGAGATTGGGCCTTGAATTTGATCATGTGATCTTGGCCCACCCCAAAGC
AGAGAGAGTTAACAAATTAACAATGATGAAGGAAAGATGTTCGTGATGAACTTTCCCCAGAAAAAAAACAAGAGTCACTGGGAAGGAAAAATCC
CTGAAAATAAAGGAGCCACATCCTGTCCTGAGTAGCACATAGTCAGAAAGCAAGATGCTTAGGGGTGGGATAGGATAACACCTCACCAGAGTTC
AGATGGAAAGTCTAAGGAAGTCCTACCACCTCTCTACCCAGAAAGACCAACTAAGGAAGCAAGCCAGGGACCTCTGCTTACAGCTCCACCCAAA
TTAAATGACCTCCAAGCTCTAAAACAATAAGGAAAGCAAATGACTCAAAATTCCAAAGTCCTTCTTTAAACTCAAGGCTGTATCATCAGTGGGG
ACATTTTAAAATTGAGGCTGCATGTCTGGGTCACATGTCAGTAGCAAACAAGAGAGCACTCTTGTCTGACTCTGCCTTGAAGCTACCTTTTCAA
TGACAACCAAGGCGTGGCACTACCCCTAAGTAGTGGTTGGTAGATGGCTGCACCTCCTTATAGCTTCTCCACATTGCAAGCAAAGGAAAACACC
AGGGAGGTAGTCTATCTTTGTTACAGCAGTGGTTCTAACAGTTGCATCACCCTGCATTTCCATATTAGTGTGTCATGCTTCACGAAATGCCATC
ACGTGCACAATTTTCTAGACTCCCGTGACAATCTCGGAAGCAGGGAACCCATTATCATTATTTTTACCCATTTGAACGAAGTTTCCTTGGGTGA
TAGACCAAGGCCACACAGCTGGTTACATGTTGGTGCTGGGACAAGAAGGCAAGTCGCAAGCTCGTAATTCTGTGAACTGCCTCTTTATTTCCAT
TGTTTCCAAACTACTGCAGGAAACAAGGGTGTATTGTTAGATTCTGGTAATTGATCAAATTGGTAGGCTTCTATAACTCAAATTAAGGGGATCC
ATGAAAGTAACAATCATCAAAACAATTTAAAATTAAGCATCTCCATCTTACTGCACAAAAATCCCCCCAAAATCCCAGGGATTTCCATTCTTTT
ACTTGTGTAGTTTAAATTTAGATTGCATTCTTTGCTGGAAAGTTGTATTATTATGCATTTTTTGAGTAGCTATTATTTTTGTGAAGATTTGCTT
TCTTGCTGGGGCATTGCCACCAAATGATCATGTGGCCTTTGCCTGGTCATTTGGCCTCAGTCTGCCTGTGTTACCTTCTTTGTTACAAGTCACT
AGCTGACCTTGGTGTCCCTCTAGCACAGTTTTGGGCTTCTGTTTCTCTAGGTAACTTGACTGTAGACTTCGTAAAGTGATCAGTGCACCTCCTT
GTATTTTCCTAGGTTTATTGTATTGTAGGTATTTAATAAATGTTTGATGATGGCATTGAAGATCCTATTGGTACACTTAACTTGAGCGATTTAT
AGGAAATCACTGTTGACCGCCTTATTAACAATTAGCTGTCTATAATGTGCATATGTAATTTTCCTTTACATCAATAAAACTTCTGCTCCTGCTG
TAGATGAAGTACTGTCCCTTCTCATCAGTTCTTTCGAATGTTAATACATTAGCTACTGATACCTTATTTGTACCGTAACTGACAGAATAATGGA
GAGAAAGAATCTAAGTAGGAGAAGGGGTTAGTTAGGTAACATTGTATAGGGCATCTTCAAAACAATAGTCCTGCTGTTTCAAATGTCAAGCTAA
TTATCAGCAATGTTTAAATGCTATAATAGGCCATTTTTGCAAATGTTGAAATTTTCCAGCAGGTATAGACATTATAAAAATGTCACATTTTGCT
ACTGTTCTTTTACATGTCACAGGAATACAAAACTCTGAAAATAAAAGTTATAGCTGTGAAAAATAAAGAGAAGCCATTACTTATTTTGCTTATG
ACTATAAAATTAATTGTCACGTTGATACAATTTAGAGTACTGTGTACTTGGGAGAAAGGAGAGAGAGGATTTAATAGAATCTGGCAAGGTGAAA
ATAATGCTTATTATAGATCAACTATGCAACCTTTTAAATACAGGTTTTTAGTAATGCGTATTCAGATACATCTTTCCGAGACTGTTTGCTATAA
AGCAAATTTAAGCATCATTTTATACCCTTTGGTAACCCCACATCACACATACAGTTCTAGAGCAAATGAAACTTTAATATGATTCTACATAAAA
TAGTAAGCCATGCCAATAATTCACATGCACTTCATATGCAAATAGTTTTGTCTTATCCATCAATTATCATGTAAACATGGTATCACATGGCAAA
CCTCCCTATAATACTGTACAGTACCTGTATATAATTATGAGCGAATTTTGCCAATGCGACAGTTTCACAGAATTGTAATTAATTCCTCTGAAGA
TTACTGTGAGAGTTTTGGATACTGATGCTGTTTTTGGACATTTATTACTGCTCTTTAGTTGGCTTTTTGCTGTGCCTCAGAGACAGATGTAAAA
TTAGTGTCTATTTTGAGCCAGCAAACAGTACATCTTCCTTCTTGTTCATTGAATTGCAGAGAGGAATTCTTTGGACTCCCACTTCTTTGTGTCT
TTTGAAGTAATTGTTTTGTGCTAGCATGTAGCATCAAAACATGTAGAAAATTGTTTTGTTTATGCTCAGGGTGATATATATGACCCCATACAAT
AACCAACCAGACTTTTCTAAAGTCTATGGAAGGATCTACATGCTTAAGTTTTTAAAATAGTTTGCAGTGTAAGTTCAACAAACAGGTACTCTTT
AATATATTCTGCCCAGTGTCCTTCATTCAAAACCTGGACTAGTTATTTCAGTAAATGCCTTTAATCTAAGAAGACATAGGCAGGCAGATCTCTG
TGAGTTCAAGATCAACCTTGTCTACATAGTAAACTCCAGGCCAGCCAAGGCTACATAGTGAAACTTTGTCTTTAAAAAGATGTATATGGTTAGA
AAATATATTGTTAGTTATTATATAAAATTAAAGCTCTTATTTATAGATAAGGTTTAATAGAAGTGGGGCTAAGTCCACCATATACTAGCCTTTC
TATAGGAAAAAAAGTTTCGTTTTATTTTGTTTTGTTTTGTTTTGTTTTGTTTTGTTTTGTTTTGTTTTGTATTTGATGAAAATTGTTCCCAAAC
TTTCTCTGAGAAAAAAAAATCTAACTTGAGGGAAAAATCTAAGAACTCAAAAGAGGGGATAAGATGGAGAGCCTGGGTGCAATCAGTGACTTTA
ATTGTAATCATCGGTGCATCAATCCAAAAGAACAGAAAGAAAGAATCTCATTTAAATTCTAAAGTAAGAACTAAGTTTGCTATGAATTACTTTA
CCTGGATTAACCAAAAAAGAGGAGGGGGGTAACACAATGGCTTATGGGCATGACTATCCTACTTTGCCCAGCCCTGGAAAAGATGGCGTACTGC
TTCTGTAAGTGGACTTGACAGCAGTTATTTATTTAGTTTAAAAGTATTTACTAGTATTTGTGAGTGGAAACCCTAAAGGTTGTGCAGCTTTGTT
GGCCTGTGAAGGTGATGGATGCTGCGCTCCTGACCCTATATGGTGAGACATGACTTCATTGCTTGTTTAGATTGTTCTGATATTTGGGACTCCT
TAAGGGCTGTTCATAAATCATCTCTCTCTCTCTCTCTCTCTCTCTCTCTCTCTCTCTCTCTCTCTCTGTGCAGAAAATATTTCAATTGTAGTGC
TGCAGGAACACTCACACGTCACATGGGATATTGTAGCCCATGCAGAAAAGGACCTTAAAAGGCCTGGGGGGTTTTGCAGGATAAAGGCCTTTTA
ATGCCTGTCCAGCCTTAGATAATTTTATTCTCTCCTTACTGGACCTTTCTTTATTCCTNNNNNNNNNNNNNNNNNNNNNNNNNNNNNNNNNNNN
NNNNNNNNNNNNNNNNNNNNNNNNNNNNNNNNNNNNNCACACTCTACCCCCACTCAGTGTTCAGAAAATTTGTAAAGAATCTTCTGCTCGTTGC
TAAGAAATGGTTAAATCTCAGTCACCAGGGGGAAAAAAGAGCGGCACATTATTGATTGTCCTAAGGTATATGTGGGCCACACAGCTAGAGCAGA
GACGTTGGAATATCTCTCTTTTTGTAGATCTGAGAGCATTGCCCAGCCTGAACTTGCCTAAGCCCTTGTGGTCACACCTCTTGAAAAGGTTTTG
TTTCCCCCGTCAATTGGTATTCAAACCAGGTCAGCAGATTCTGTTTGCCCGGCTCTACTGCCGTTTTCATTTTAATTTGCACCTGTTGTGCAGT
TGTTCGGAAGGCAAGTGCAGACCCGCGGATTATACAAAGATCATATTATGAGCAGATATGCACGGTGTAATCTTTTTGTCCCCAGCCTCACATG
ACAAAAAAAAATCTCTCTCTTTTTGTTCCTTTTTTACTCCCTGGGAACCTGTCAAAGCAAACAGATATGACTGACTAAAATTTGTAACTAGACA
TGTTTCCAGAATTCGCCAAAAGCATTATGAACCTGACACACAATCCTTTTCATTTTTTTTCCCTCCTTTTCCGCCACTTTTGTATAATCCGATA
GCTGGCGAGAGCCACTTGAGATAGCTCAGTTGATAACTTAGCGCAGGATTGCTTTATTAGGCTCAGAGAAATCAAAATGAACCTGAAAATTGTT
TGCGCTTTTTTTCCACTGCCTCCCCCCCTCCCCGTCTCTCTCTCCCTCTCTCTTTCTCTCTCCCTCTTGTTCCCATGATGTCATCGTTCTGACT
TGTTTTTGTGTGTGTGTGTGTGTGTGTGTGTTTCTGGAGAGTTGGGTTAACAGTTCTTGGCAATCCTGTGTGTGCGTAACGGCTGGTGTGTTTC
TCTAGCTGAGCTAATGCCCCGTGTTTATTACTCTAATCTTTCTTGTCTGGTGGTAAAGTGGACAATTTATGTACTAGCATGTAGGCCGAGAGCC
TGTGAGGGCTGACTAATTCAGAAACAGCCACCTTCAATTGAGTTCACAGACCTTATTTACAGGCTGTCTATTTTAAGAAAAGCTACTGAGCGTT
GTAGGGACTGCAGGCTGCAGAAGCAGCCCGCCCGCTCTTGTGAATTATTTTAGCATTTGTGTTGGTCTTGCCCGTGTCACTTTGAAATCTTCTG
TAATTATGACACTATGAAGACATCTCAATAGTAGTATTAATGGCCCAGAAGATATGGCTTTTCCTCATTTTCTGCTTAAAAGATGAGGACATAA
ATGACCTTAGCTCATGACGGTTGAAATAGAAGAAAGACTGAGCATTTCAGACTGTACTGGTTTGTGTATGTGTCTGTGTATTGGGCAAATAGAT
GGAGAGAAAGAGAGAGAGAGAGAGACAGAGAGAGAGACAGAGAGAGAGAGAGAGAGAGAACTGTTTCTAGGAACAAAACTACAGTTAAATTACT
GAAGTCATCTTGTATTTATAGTACAACCTTATATAGTAAACACGTCATGTCACTGAGCTAATAAAACTGTCAACTGTAGATCTATTTTATAAGT
TTTTGTGAAGTGTGGAAAGTCCAAACTGCACTATGTCTTATTATGTGTGCAATATAAGCAGAATTTAAGCATGTGTACCTCTGTTGCCTTTAAA
ATTTTCCATCGTTTTCAGCAAAATATTGAAAATAAAAAGAGTACACAGCTATAATAGAAATAAGAATACAAATGCTATGCATTGGGGATATTGA
TCATGTTTTCCTGACATGGTTGAGTAATTCAGTTACGAAAATATTAATTATTTTCTATTCACCTGAATATGTGGGGGCACTGGTATTTTCAAAG
CAGCATTTTGAATGTGCCTGTGCATCTCTTCCCACAGTGGTGAACTATGACAACGTAGTGGACGCAGGCTCGGAGACAGATGAAGAGGACAAGC
TTCACATTGCTGAAGATGATAGCCTTGCAAACCCTCTGGACCAGGACACCAGCCCAGCTAGCATGCCCAACCATGAGTCCTCCCCACACATGAG
CCAAGGGCTGCTACCAAGAGAGGAAGAAGAGGAGGAGCTAAGGGAGAGTGTTGTGGAGCACAGCTGGCACAGTGGCGAGATTCTGCAAGCCTCT
GTAGCCGGTCCAGGTAAGTGTCTGTCTGTCTGCCACTGTGTGGCCCCTTCCTGGGCCACCTGCATGTCACCTGTGCTTCCCAGCAGAACACCTG
CAGCCACATCATTCAAATAGTTCTTTCCATCGCCCTTCTGGAGTAGAGAGCAGAGCTGTTGGCAGATGATGAAGTGATAGCAATGTGGTCACAT
CACGCACAGTACTCGGATTTTAGGAAATGATTGTGTATTGGTTTCAACCCAAGTTGGATATCAACTCTAGTGTAACAAACACAAAACATGAAGT
ACTTGTACATTTTCAATGTGAAACATTACAGAACGCAATCAAGATCTCTACTCTCATGACAGAAAGTATAAAGAAATACAAAGGCTTCTCTCTC
TCTCTCTCTCTCTCTCTCCCCCCCCTGTGTGTGTGTGTGTGCGCGCGTGCGGGCATGCACGTGTGTTTTGTGTGGGAATTGTGTTATTTCAAAC
CACATAAAACTGTCAGTTCGGTGTACTTCATGTCTGACACCTTCTATTGGAATTCCTTTTAATTCTATTGTAATTCCATCTAATCATTCTTTTT
TTGTTAAGTGTGAATGTGATTTCACAGTTTTACCTGGTCAGGTAGCTTTCTTACTATTTAACAGTTAAGTACTCGATAATGATTCTTACTGTTT
GATGGTCATCTTAAGGGAAGTTAAAAAACAAAACAAAACAAAACAAAACAAAACAAAACAAAACAAAACAAAACAAAACAAAACAAAAAAAACC
CAACCAAACAAACAAACAAAAAGACCGGTCCAGTGCCAACTGTGATAGAAGTAAAACAGATCAGTTTCAATCTGAAACTTCCGATGTCACTGCA
CAAATGGAAATGCCTGAGCCTGAAATTCCCCTGGAGTGGAAGACAGGGCATGCTAACGAAAATCCAAGGGCCTGCCTCGGAAGCTTAAGTGGCC
TGTGAGAAGTTTTTTAAAAGTCCCCTCTAATTCAGACTTTGTGTCTCCTTCACCTCTATTCTATGACCTAATAAGAATTACCGTTATGTAGCCC
ACCTTGGTTTTGCGTTGACAATATAAATAACTACTGTTTTTGTTTGTGTGTGTGTTTGTTTGTTTGTTTGTTTGATTGATTAATGTGTGTAGCG
GGCCAGACAGAGTTTAACTTTACCTCTCTGTTATTGTGGTGTTGCAGGCGAGCAAACACTGTACAGAAGCAGAGCAATTGTTTGCTGTGTGGGA
AGGATAATAAAATCAGGTCTACTGGTAAAGAAAATTAAGTGTGTGTGTGTGTGTGTGTGTGTGTGCTCGCGAGTGTGTATGTATGTGCGTGCGT
GTGCGTGTGCGTGTGCGTGTGTGTGTGTAGGGGAAGACTAATGGCAGTTTGACTGTGGTGTATTTGTTTCTCATTTCCCTAATACACCGTGCCA
AGTTTGGTATTCTTTCTGTTTCCAATTTTGCTTTCGGTGAAGGTTTCTCGAATCTCAGCAATCCCACACATAAACATTTATCGTTACATCCATT
AAGACTTAAGGTTTTAGATTTTCAACTATGAGGGAGAACTCAGCCAAAGATGCTTGAAAGATAAATACGTTTAATTAGGGGCAGAGGATGATCA
TTAAAGGAAGTCTGACTGCTGCAGAAGTCATTAACAATCTTAAATGAAATTTTTATTTTTATGATAGCCATTTACATGTATAATAAGAGCCAAT
GATTCTTGGGAGCAGTGTGACAGAAACAGAGTGTGGCGAGAACTCATGTAAGACATACTATTTAAATGAGCTGGTGTTAGCTATTCATATTTGT
TAATGAACTAGATATGCAGGGCTATAAGAACAAATGTTCGGATGCTTTAAATTTTAATATCCAGAAATCAAGGGTAAGGACCAATTACCAGAAT
TTTCACCCTTAAAGGAATAATTAATAACAATATAATAAATGTACAGCCTCAAACTTCTTTTTAAAAAGTTTAATTAAAAAAAGAAAGAAAGAGA
GAGAGAAAGAAAGAGAGAGAGAAAGAAAGAGAGAGAGAAAGAGAGAGGGGGAAAGAAAGGAAGAAAGAAAGAAAGATAGAAAGAAAGAAAGAAA
GAAAGAAAGGAAGGAAGGAAGGAAGGAAGGAAGGAAGGAAGAAAGAAAGAAAGAAAGAAAGAAAGAAAGAAAGAAAGAAAGAAAGAAAGAAAGA
AGTCGATAGTCGAGAAACAGCCAAAACAGCACTGTCAGGCTATAGTAGTTTGTGACTGTAGCAGTTTTTTTGTGTGTTGGTCATGGATCATAAT
CTCAAGCATCTCAATGTACAATGATCCATACGAATCCCTTACACAGCCTGCTGTCACCTGGAGTTAATGGAGACAGTGATGTAAATGCTCTTAC
CTTTATAGTAAATAAACAAGCATTCTGGAGTCCAAATAATGAAATAAAATGTAAATCCTAATCTCTTTAGGACAAGAAAGTGACCCAGCTATTA
CCTGTCTCGTTCTGATGATTAGTGATGATTTACTGTTCTCTTTTTTATTATTGTTCTAAAAGCGCTCTGATGTGGTGCCTAGATGTCTAATGAA
AAGAAATTAGTCTTAAAAAGCTCCTCATTGTTTATCCTGACTTGTAGATAACACATTTTTCTTCTGATAAAATATTTTTCTTATTTATTGTTCA
AAACACACACTATAGACAATAACTCTAATTCTGGTGTGTTTTATTTAGGAGTGTTTTCTCCCCGGTGCCTGGCTGTCTGCAGTCCTACCTCGGC
ACATAGCTTTCCCCTGAGATAAGCTCCTCTCCTTTGCTCTTTCTCCCCTCGCTCTGTAGTTTGTCTGCAGGATGCAGGTTTTTGCACTGAAGAG
TGAGGCAGAGTTCTTACTGTGCCTTTCCGGTCCTAGCACAATCAAAGTTTTGTGTTTGGAGCTGTCCTTGACATTCTTTCGTCCCACAATGGGG
TGGACACAGTGTGCTGGAAACCATGACATCATTATCATGGACACAGACTTAAATAAGTCACCTGCATCTCAGCCTCTTTCTGCTTATTCAAGGA
TTCTTCATCCTACAGAAGGAAGTGTTCTTTATTTTTACAAATGCACCCTTTAGCCCCAAGGGTGACTGGAAAGTGCAAACTTTTACAAGTGTTT
GAAAAATAAATATACTATGTGGTTTTAGACACCGTAATGCTGAGTCAGAGGTGTCTTGAAAAAGGTTAAGCATGAGGAAGGCGGCCAGTGAGGC
TTGCCAGGAATTGAGGGGAGGCATTCCTAAAGTGCTATCATTATGTCCTCTTGGATATGGATTCACATGGACCCTAACACCTGAGAAGGCTCAG
TGACTCATCTAACTAGAATGAGCTTAATATTCCAAAACAAAAATGGCCGGGCTCAGCAAGTCAGCTGGGGTAAAGACATTGCAGGTAAAAGTGG
CACCAACATCTCTAAACAGTGTGCAATCTTTGGGAAAAGCGGGTTATAGCTAGACAATGCACAAAGGGCCTACTATTCGGCTGTGCAAATGACA
TTTATGAATTTGGGAAACAGCCAACTATTGTAACTTCTCTTACCTTTTTATAAGAAAATGACTATGAGGAAGGATGTCCAGCAGAAATAGCCAA
TACTACCACCATTATTTAAAGAAGTAAAATATAAATAAATAAAAATAGCTGTTAACCGTTTTCCCTTCGGTGACTCAGTGTTCTCTCAGATGGA
AATCTTATGATTCTTGTGACTCTTGGTTACCGGAAGTCATGTGAGAGAGAGGTGTGAGGGCGAGAGGCCCACAGGCTATATTTATATCTAGTAC
TGAATGCAGACATACCTATTTTGTTTCTTTTGGGGACCAATAAAAATACCAAAAGAGCTTCCCAAAACATTGACTATTACATTTGACAAACACA
CCGTTCCTGTGTATTGAACTTCCAAATGTTTCATGCAAAATATCCCTGTGAAAATATTCCTGGAATAAAGGAGGTGCCTAAGGTCAGGAGTTAA
TTTGAAGTGGGTGCACAGTCTAAATAAATATTATTAAAATTATGTGACTGTTTTGAAAATGGCATTACATTACTCTCTCTCTCTCTCTCTGTCT
CTCTCTCTCTCTCTCTCTCTCTCTCTCTGTGTGTGTGTGTGTGTGTGTGTGTGTGTGTGTGTGTATCTGATAGGAATGTGCTCTTTCAGCTTTG
ATTCTATATACAGATAGATTCAATTTTGCTATAACGTTTTCTAGCCACTGTTTTTCCACCAACCACAGCAGTGTATTATGTGATAAACAAAAAA
ATGATCAGGATACATCTGAGAAAGAGTTCGACTCACAGTTCCTGGTGTTCTAAGTAAAAGGATTACAGTACAGAAATCCTCGCCTCGGATCATG
GTCTGCTCTCATTAGTATTGTACTGTACAGCTACAGTTTCTGAGAGTCCATGTTATGTTTGTTTATTTGAAATATTTTTAACTGGCAGGCTGCC
TTGCTCTGCCTTCCCACATACTCTATAATATAGTACACAGAGAAAATTCCCATTTTTCTCCCTTATTTGAGAGTATTACCATTTTCAAAAATGG
CCCTTAATCATTCATGAATTTCCACCTTCAACATTACATATTTTCATCTTAATTAAAACATTGAGCTACTGTTTTCTCTCTATTTTTTCATCAG
GAAATGAATATGATTGCCTGTGGAGGCATAAATGCTCAAAACAAAAAGCTATTTATAGAAATGGGGAGGTATGAGAAATAGACTTTAGAAATTT
ATGTAAAACAGTCAATGGTTATTTTTAATACATCTGAGATGTTAGTTTGTAAAATCTAAGTATTCCAAGTTATGATATGCAGAATTGGGATTTT
CTTTTCTTTTTTTCTTTTTCGAGACAGGGTTTCTCTGTGTAGCCCTGGCTGTCCTAGAACTTACTCTATAGACCAGGCTGGCCTCGAACTCAGA
AATCTGCCTGCCTCTGCCTCCCAAGTGCTGGGATTAAAGGCGTACACTACCACTGCCCAGCAGAATTGGGGGTTTTCTTAAAAGGATACCAGCC
TATACTAACTCCATCATAGCTATACCAAGCAGAAAGAACATGAAAGCAAAATGAGATATTCTGGCTTGCCATGAAGCACCCAGAGACAGCAGCT
ACTGTGTCTCTTCATAAGTTCAAATACTTCGTCACTTTTGACTCAATCTTATTGAAAAAAATAACATCTCCCCCCTGTTTGTCACTAGAACTGC
CACCAGAAGAAAAGCCCTTCGTTTGCTTCAAATAAGCACCTCTGTTTTTCACAGCTTGCAGTAACGCTTAAAGTAAATAAGTTCTTGTGCAGCC
TGCACTGTTGAATTCATTCAGATGCCTGAACAAAGCTTTTGTTTAGAGTTTTACAACAAGCCTCACTGTTTCCCTTCAAAGACTCCTCCTTGTT
CCTATGTTTGGCATGCTTAGAATAGTAAGCCTTTCTGTAATTTTCAAATGCTAAAGATAATTTTCCCCCACAGAAGAAATGAAGGAAGATTATG
ATGCAATGGGGCCAGAGCCACGATCCAGACCACAATTAACAATGGTACAGGTAAGTTACGTTCACATAGCAJACATCATGTTTGTTCTGTGTCA
TGTGGTTTTCCTGAATCTGATAAAACACAGACACATCCATTTGCACCTCCATGATTAACAAGAAAGTTAGACTTCAGGGTCTAGTTTTGTTTCA
ATTTTGAATTTGCCATATCTCTTATTTTATTAAGCAACTTTGTCTTCCATTCTTATACCCTAACTCGCCTCTATAGCTCGGGAATTTTGAGTTT
TATGAATGGACATTCTTACAGCATGATTTGTATGGCTTTCTAGTTTTAGCTTTTACTTTAAACATAATTTTTATATCATAATATAATATGCTGG
CCTTAGCTACTACTTGATATCTGTCATTGAGAATTCAAGTGATATGATGAAGTACAAATTGAAAAGTACCTTTCCCAAATCCACATTTCCATAA
ATTCCTTAAAAAATTTTATCACTTAACTTCTCTCAGATTCTCTCTTCACTTTTAAATCTCTGCGTCTACTAGAGGTATCAAGTAATATTTCTCA
GACAGACTTTTAAACAAGAATTAAATAAAACTGATGAATAACCACTCAGGACACAGTAGGCAGTGTTTTCCTTGGCTCTGCTGTGGCTGGTTAA
GGGTGGCATTCCATGTTCGCCCCCAAACAGCATCTCTTCTCACAGCACAAGGTGACATAGATTCACTCTGTGTGTATCCTTTTCTGTATGGCAC
TACCTTCATGTTTCAAACAATCCTCCACACCATTGTAACACCATTACAAATGTTAGCACAGTCCCCAATACAAAGTCATTCCCTAAAATAAGAC
AAGAACAAAAATCACATAACTGTCTCCATTCAGCGTGCCAATGAAAAAAGTGTGCACTTTAAAATGGCTTCAGAAGTATATTTAATTAAGATGA
CATTCACATTTCCCTTAAGTCGATATGCATCATAATATACTGTTCATGCTGTCTATAAATATGTTTATTAACTACTTAAGAGATATGGGTTCAC
TGTCTTGGGTATAAAGTAAGTGAAACAATTTTCTAAAAACTCCTGACTTACTCATTTGAGTAATGTCAAATTGAGTACAAAACTAAAAGTATAC
AACCATATGGGCTTAGAGAAACTGTGGACTAGAGCACTGATAATTCTAATTTCTAGATGCAGCTTCAACAATACCTAGATTACAACCTTAGCCA
AATCAATTAGTCTCTTGGGAACTCAGTTTCTATATCTGTAAAATGCATATGTTGAGCTAGCTGACTGCTCAGAATGCTTCTAGTTTTAATATTC
TATGTTGCTGGGATACTGTTTCTATAAAAATAGAAACAAAAGCGAAATGATTTTGTTTAAAATATAACAATGTACCCTAAATTAAGGACTCTCC
AAAACCTCCCAGTTTGTGAGCTTCAAGATGGCCTCAGGTTCCCTTGAAGAAAAATGTCTCAGCTTCAAGCAAGCATTCATAAAAGCTGATTCAT
TTCCTTCCTGATCACTGACAGTGTGATAAAGTGACAATTTAGCATCCATTGTATATACCTGAGTCTAACATGAGGTTAACAAAAAGTACTCAGG
TCCAGGAGGTTTGCAAATCAATTTTGAACACCCTTTCACCAGGCTTTAAGATCCCCTGTCTCCTAATACTCAGTGATTCTTAGACTTCATATGC
ATGCAGTCAATAAAGAAATCAATACATAGTCATTGAGTGTTTTCAATATAGTCCCTGTTGTGTGCATGTGACGGAGATGATAAGCCAGGAGACT
AAGTCATAATTATTATTTAATTTGGGAGAGATGCCCAATTTACATGAAAAGATGATAAACAGTAAGTGAAAATATGTGATAAATCAAATGGGAG
GAGTCATAATTATTTGAATTACAGAGATTGACAAGATCCTGCAGAGTGGGAATCTGTCTAGGTTTTATGAAGGAAGTTGAATTTGAACTAAGTA
CATTTTCAGTAGTCTGGAAGGAAGAAGTGGAATATATAAGGTAAAGAGAAAACATAGTATCTAAGTAGAAGATAATGGGCAAAAGATATAAATT
AAAGGGAATTTATGCGAAATAAAGTTATAAACCGAAGACAACTGTTAGAGAATATAAAGAAAGCATAAGAAGAAAGAAGTTGGGTCATGTTGTA
CCACCATGATGTCTTGGGAAAATCAAACTATAGCCGTGTAGTCATAAAGCCATGAAGTGATTTTTAAAAAAAGTTTGTCATAGGCGGAAGTGGG
GAATTTTCAACAAGACTTAGTAGGCTGGTCTTTAACTCATAGAGATGCACTTGCCTTATCTTCACCAGGACTGGGATCAAAGGTGTGCGGTCAG
GCCTGGCCAACCCAGCATTTTTACACAGGTTCTGGGGATCAAACTCAGGTCCCTGCTTGCATGATAAGCACTTTACTGACTCAGCCAGATCTGC
AGCCATTGACGAGACAATTTTGAAAACCTTTGATGTCCTAGAACATCCCACATCAATTTGAACATCTCTGCCCATTGTTCTGCTGTAGCATTTG
AGGCTCAAGTAATGAAAGCAAGTTTGGAAGGCTCCTAGAAACTACCCGCATCTACCCAGTGAGTCTCCTCACCTCATCTTTAACTAGCATCCTT
AAATTAATTATAGAAGAGGAAGGTTTAATATTATTCATATATAATAATGATAAAGCTGTCAAATATTTCTTTGTTATTTCATAACTGATTACCA
TCAGGATTCATTTTTTTTTCTTCAGCTTGGATAGCCATCAATAAACAAACACTCTTTTCTTTCAGTATTCATCTTAAGCAGACAGAATTTAAAC
CGATGCTTGAGGGAGGGCAAACTTTAAAGAAATGGAATATTTAATATGTGCCCTTAAGAAACATAAAATTTTATAGGGCAATGGGGAGGTCAAG
TCATAATAACAGAAAGACAACATTTGCAAATAAATAATTGTTTCTTCATTAGCTCTGCAGGCCAGTCATCATTTATTGAGACCCTACTAAGTGT
AGAAGTATTTTGTTCAGCTATGAGCACAGAGCAATCAGACAAGTAAAAGAAAAGGCACAGGGATGAGAAATATGTGCCAAATATTTAACACAAC
ATTGACTGATATTTATTCATTGCTAACCAGTGTTATAATTTTTACTGCTAGTAATGTACGTATGTGTTACATTGTTTCATTTCAAGTACAGCAA
GCTACGTAGGTCCAAAGGCAAAGTGTATACTGCAGAGTTTCTTGACAGAGAACCGGGATCATATGTTAGAGCTAGAGCCATAGACGGATGCCTG
AAGATTTCCAGGCAAGTTCTTCAGGAGAACAGCTTCATGAGCATGCCTGTGTGCAAAGAGAATAATGGAAAGTGGCTGGACACAAACGAGAGGA
GGCTGAGGAGGCTCTTAAAAGAGCACGATGGCAACAGTGGTAGCAGTTGTTTTTGTCTCAGTAGTGTGATCTGAGAGAAAAAATGATACTTGAA
ACGAATTAACCAAATTTAAGGTGTACCGGGGAACTCTCTCATATAGCTTATACTCTTGAAGTGGACAGGAAAAAAAAAAGAGCTTCAGTTACTA
GCAAATAAGTAGGAGTATGGATGAAATCTCAAGTAGAAATGGAGAGTAATTGCTCTGGACAGCGTATACAGTCAGTCATCAAGAAAGAATATTC
TGTTTTGAAAACATAGCCTGAGATTAAGCATATAAATATTTAAGTCTCTTTCCATATGTTTGGTACTGTATTAGACACATAGATAGGAACAGGA
TACCTCTTAGTGTGCTAAGGTAAGAGTGGAGTAAGAATGAAAAGACACATTTCAAGGTGAACTAAGGCAAGTGCCCAGAGCAGACGGGGGATCT
GTCTGCGCCTGGTTATCAGTGAAGTGCCTGCTTAAGGAATGAGGAACTCAATGATGAGGATGGGGGATGAGAGCACGCATGTTCCTGGTTCTGG
TTCCGACAGATTGAACTGCCTGGAGCTTAGGGAAGTACCAAGACTGAGGGGACAATTAGGCACCAACTACTTACTACCTGTTTGTTGGCTTGTT
TGTTAGTTTGATTTTTTTGAGCAGGGTCTCAATATGTGGCTCTGGCTCTCCTGGAACTCACTGTGTACAACGGACTGACCTTGAACTCACAGAG
ATCTGAGTACTTCTGCCTCCTGAGTGCTGGGATTAAAGGTGTACACTAACTTGCCTGGAAGGACTACTACCTTTTAGCCAAGGGCAGCCTGGAC
AAAATTTAGTCCTAGGACATAAGTCATTTTACTTGATCTATATAGATAGTGCACTAAAACATGCTGCATAGAAAGATAACTTTAGACTTCGGAA
CATTATAGTTATAGCCAGGCATGGCAGAACACTTCTCTAACTCTAGCATTTGCGAGCCAAAGGATGGTGAGTCTGAGGCCAGCCTGTGCTACAC
AATGAGCACAAGGTTCTGTCTGAAAGAAACAAACAAAATGCTTCAGAAACTTTATAGAGTGGAGACTCTGAGGGTAGATACTAAGTTTGTTTTT
CTCCATAGTCCCCTCAACATGCGCAGTACTTTATGTAGTGCTGGTGGTTGATAAGCAGGTCATATCTTGAGTTAGAGAATGAATTAATGATGAA
TGAGCTTGTAAAACAGGGAACAGTGATTTGGATCAGCAGTGTGAATTAAGAAGTAGTTTGACCCAGCCTTTTTGCTCTGTGTTTCAAGAAAGAT
GCAAGCAGGCAGGGCTCTGAGGGAGAAGCCTATGAGGGATGGGGGATTACCGAGGGCTGACACAATTTCATTTAATCCGAAGAAGTGGAGCTCG
TGTAGGAGGATGTCATTAAGCATGTGGGGGGGAAAACAAGTAGTTTGTGAAATAAGAAAGCAAAACGAAGCGTGAATGAAGTTCCTTTTTGGTC
TCTTACTCTATATTCAGTTTAAGACATATTTTCTTTACTATGCACTCAGACTATTAAAATAATCGTTCATAATAATGTATCTTTGCAAGATGCT
TCCAAGAGCTATAATTTATTGAGTACATTTAAAACTTGATTAAAAATAAGAAATACTGAAATAGGGAAAATACAGTCCTTATATATTAAATAGA
AAGGTTAAGCATCTCTAGGACAGAATCTGAAAAAATTCTTCTTTTACCCCATTATCCTGGTTTGGTTTCTGTTGCTATGATACGCAACACAACT
AAAAGCAAGTTGGAGGAGAGATATTTGTTTGGCTTACAGGTCATAGTCCATCACTGAGAGAAGCCAAGGCAACCAAGGCAGGAGCTTGAAGCAT
AAACCAACAGAGGAATGTTGTTTGCCAGCTTGGTTTCAGGCTCATATTCAACTTGCTTTCTTAATACAGCCCAAGCTCTCTTGTCTGGGGATGG
TGCTGCCCATGGTGGGCTGGGCCATCCTAACAGGAGTTAGTGATTTAGTAAATGCCCCACAAACATGCCCGCAGATCAACCTGATGAAGGCAAT
CAATCCTTCAACTGAGATTTCTTCTTCCCATGTGTGTCATGTTGACAACCAACCAAAGTTAGCCATGATGACATGCAGTTACTCAATCTGGATA
ACCATAGAAACAGAAGAAAAAGGAAAGAGGGGCAGGGCAAGGAAAGAATGAAAAGATATAAGATTTATGCAAGTAGGGACACTTTCAGCAAATA
AGTAAATTTTAATTTACAAAATAAATCTACAGACATAAAGAGGAGTCATTTAATATCATATGCCACTAAAGCTGGCATTTTAATTGGCTTTTTT
AATACTAACTTTATAGTGTAATAATGTGGCATTTAGAATTATTCATGTCCTTGTGATCTTTTTCAGCATCTAGGTCCTTTTCAAATGTTATTAA
ATCAAGCATGATAAACAGCAACTACAGAGATGGAGTCTCTCTCTTATGTTGAATCACTGTGATGTAGTTGAAAGAGCAATGGGCCAGGAGTCCA
GAAACGTGATGTCTGGCTCCAGGTCTTCTAGAAACTTCTTTTGTAACTGTGTGAATCGTTAGCCTCACTACATCTTGGGGGCATCAGCTTCTTA
AATGAGAAAATTAGGCTAGAAATTTTGGGCTTCCCAATTCTCATCCTTTCTCCTCATGAAATTGCCTTGGGAAACACAATTTAAGAAAATAAAA
ACTGTCATGATACATGGGAATGGAAGTCCTGTAAGGGCCCTGCTCTCTTCTGCTCTCCCTACGCAGTGAACCTCGCCTTGTAATTGGTCCCTAA
GCTGCCCTGTCTTTGGCTTTTCATTCTTGATCATTTTAAGAACTACAAACAAGGTACTCTACAAGTTTCATATCCCACCCCTCCCCGCTCCCAG
CCCTACAATTCATCTCTTGATGTCATTTCACAGAACGCAAACAGCAGAGTGTAACTGTAGGATGAATACTACAGAATCATATAAGGGATTTATA
CTCCCCCTAAAATAACAGTTCAGTCCATAAAATAACAAGACCAGTGTTCTCATAGCAAATGAACCCTCAATAATAATCCAGACCAACAGTGGAA
ATCTCATCTGAGCCAACAACACAGTAGTTTGACTCCTCTGACTGGGTTAAGATAAGGCTTATTTTCTTTGTGGTTTAGCAGCCCCAACATCCTC
TTAGTTCTCCTACAATGGATCTTAATTAAAGGTTTGAAGTTTTCTTTTTAGCATTTTTTTTAAGGTTGGAGGCCATGCAAGTTTGAACATCAGG
TATCTCCCAGCATAAGATGAGAGACTGTGACGTATTAGCAAACATACAGGAGTGGAGAATTCAGTTAGTACCCAAGTTCCCAAAAGGAGGACAC
ACACTACTCTACTATCTTGCTACTTACTGTGAAGCAAGTCTCTAGTTTGACTCTGTCCTGTTTTTCAAGGCTGGGGAGATTTTGTCATTCAGCC
ATACAATTCATACAATTCAGCCATACATCTGTCATACAAATCATGAGTTTGGGGTGTGTGTGTGTGTGTGTGTGTGTGTGTGTGTGTGTGTGTG
TGTGTGTGTGTGTGTGTGTTTAAAGTAAATCTCCTATTTTAAAAAAAATCAATGACAAAAAAAAAAAAAGTTGAGAGGAGAAGGAGCGCTGGAC
AGGACGGACAGGAATTGTCAGGGATAACCCATCACAGGTGTGCACCTGGAAGAGTGCCTTCACTTCCTGGCTCATTAGTGCTGGGGTTTTATTA
GCTTCATCTGCAATTAGGAGCAGCCATCTCTACTGCACAGCAAGAGTTCCCACAAGAAATAACGACCACTCCTAGCCTCTTCTTGCATGCCTCC
TTCACTCATTTCAACAAATATTTAATAAGCACTGTTCTCCTTGCGATGCCGCCTGTGGTTTCCAGGCATCTCCAAAATATTTTTTCTTAATCGC
TGTGTGTGGTTCTCCCACCAGAAATCTGTGTTATTTTGACTTTTTAATTGAAAAGAATACAGATGAAACTCTCTAGACTTACCTAGGGAATAGA
CAACAAATTGATTATGGCAATTAATTCAAAGTATGGATTGTGGTTACTTTAAGGATACCCAAGGAGGTACATGTTAGTTCATTTCACAGGTGAG
AAAATTACGAACCAAACCCAAAATAAGTGGTCCAAATGGGGAGTCCCAGGCTCTGAGTTTGATATTATCTTTCAATTATCCAGAACTAGATGTG
TTCTAGACTTCCCAGTAAGTACTGGAGACACATGGCCCACACCTCTGACAGTTCATATATGTGGAAGAGAAAGATGAAATGTGCTATGAACCAT
TCAAGGGTTCACATGTGCAGATGGTAGCCAGCAAGCAAAGCAGAAGAGTGCAAACTCAGACTTAAGTGCTCAGGAGGCAGCCCGATGTGAAGTG
ATCCCGTGAAATTACTCAGAACCAAGCTGTGTGCAGTATGAGACAGAAAAGAGAGTCCTACCAGTAGAGATTCTTAAGTCAGACAAGATTTTCT
CTATGGCCTGTTAAAACACAGACTGCTGATTCTGTAGTGGGCCCTGAGAACTTGCATTTCAAATAAGTTCCCAAGTATATTGACACCACCGATA
TGCAAACCAAACCTTGAAAACCAATGACTATCCAAGGCAAACACTGGCTGTTTTATGGCTTTCTATGAAAAGAAAGTATGAACTGCTGAGCAAA
GTCACTGTCCTAAAGACAGCAATGCTAATTCCAGATAGTTAGATTACATGGTTAAGGGAAAGATGAACACATCTTGTTTAGCTAGGGAATTAAA
AGTCAATACGCTGCATCATTTTGTACTGCCGCACAGCAAAGTCATTCAAAGGTTACTGTAAGCCTCTGGGTCTTGATGTTCACAATCCTCTCAT
ACAAAATCAAACTTTAAAATTAGATGGCATCCTGGACTGGGCTACTTCCTGCTGTGGAGAGAAATATTTGATCTTGCAGACTGCTACAAATTCC
CACCTCCCATTCATTTAGCATGAATCTCTCACTGGTATTTGAACATGCCCCCTCAGAAAAGGCCATATAAGAGCAGGGGTGGAGAGACCACCAA
GATGTCTAATTCTCTGCAAGAGAAGAAACTTCTCTGTTGCTCTTGACGACCCTTCTCTGGCCTGATACATGCCTCTGTACCAGTCGGAGGCTCT
GAGAAGCTGCTGTGTGTCTGTGCCTTGAGAAAAAGATCATCCCCTTCCCCATTTTTGCCAATATATGTATGGTGATACTTAATTATTAGCCAAC
TTTATACAGAGTAGTGAAAATTCATATACCTTTGCATAAGCTTCTTTAAACTAAAAAAAATCAATGTAAAATTAAATATTGCTCTGTCTTCTTT
TCATCAACTAGTATTAAAGGTGTTGTTTTTTCCCAAGGGTGAAGACGGTTATGCTTGACACGTGACTACTGCTTCTCTTCCCTTTGCCTTGACA
TAGCAATGTGAATAAAAGCTAGATTTTTTTCCAAAAGACTTACTTATTTATGTTAATGTTTGCCAAAGTGGCTTAGTAATAATATTAAAATATA
TCTCTTCAGTTTTTAATGAAAATTGAAAACATGCCAGATTTTAAATTAGTGGTAACAGAGAAGCTGCTTTCTTGTTCTAAATACAGAAAATAAA
TGGCAAAAGGAAACAAGAAAAAAAGAAAAAAGAATCAGCTTCGGGGTCACATTATAAACTCGACGGCCTGTAAAGTATAAGTAAAAATCACTTT
TAGTGGTGTTATTGAGAACATGTTCTAAGCAACTCAATTATGGGAACTGACTGATTCCTGAGCCAGTCAGTGGTCCTTTTCTGCTGACCCTGCT
TGTTTTTAGGACAATTACTTTAAAAGTCAGTTTAGACTAACATTCGTTATCTACCTTAAAAAAAAAAAAGTCAATATTTTCCCAAGACAGTCTT
AAAGGTATTTATGAAAGTATCATTAATAAGCACGACTAGTAAGACTGACTAACATATTTAAAAGAAAAGAGAGCCTTTGATGACTTGAAGGGAT
CTAGAAATTTATCTACCAATATTAAAACACATATAGCCATGAGGAAACATTTTTAATTAACATTCTCTTTTTGGATGGAGAAAAGTTTAACTAA
CCTCAGGGAAGGTTGAGATATTTGCACGACGTTAGGGTAAATTTTAGGAGTAAATGCAAAATCCCCGGAGAAGATATGGGAAGAAATTACTTGA
GGTTTCTTGTGAATTTTTTGTTATGGTTTGATGATAAATACCATGCAAATATCCTCTGTCAGTGTGTTTTGGCAGTCCTTCCAAGGCAGCCAGG
GAGTGAAGAGGATTATAGAAGGAACAAAAATGAGGTTGATTTTATACTAGAAAAACACAGCGGGGAATTCTGTAACTTCTTAGCAAGCTCTCCA
CCTCAGTCCTGTGTCCACTACACTGTACTATAAAATCTTTGGGAAAACAAATATACAAACTTCCAAATACAATCAAAATTTCATCCTTGCCAAC
AGAGGTAACCAAAATGCACCCTTATCCCTTGCTCCAGTACTACAAGCAAGAAGGGTTTTGGTTTTCAGAGACTGGAACATGGCGCAGTTGAGAG
TAGAAAAGGAAGTTTCTGGGGCGTCATATATAAGGAGAGGTGAGAGTACAGTCACTAGAAGATGTAGCCCAGCCTCAGAACACACAGCTGGATG
TGTGGCTGGGGACCATCTTGTCTACCTTCCCCAGCTTCCCCCCAAACCCTCCACTCCTAAATGGAAAATATCAGTCCCTTCACTCTGCATCTTA
CTGCTGTAGTCACCTGGCAGTGGGGGAACCCTGAGCCCGGATGCTCATTTTGGAACTAAGTCCTAAAATCATAGGGAAGGGATCCATGGGCTTG
CATTGTCTCTTGGCCTGTCCTGGACAAATTAATGATTAGGCTTGTTTCAAGTGCAGGTATGCAACTGGGAAAATGGGAGGGGGATCACACTGGA
CATAGCACTTACTTGGGTACTGTAATGCTTGGTAGTGTGGACATGAAGAGCTAGATCTAGGCAAAGGAAAATTCTAAAAAAAAAAAAAAAAAAA
AACAAAGAGAAACTTACATTGCTTTTCATGGTGGATATGATGGCTCATTTAAGTTTGTATGTTAGCTCACTCTAAGTGGCTGTGCATAGATGAT
CGATAGCAGATCCGCGCCCACATGAATGATGGAAAGCCCTAATGAAGCCAATAGACTTAAATGATTAAATGACATTTCCTCATGAAGCCCCTTT
TGGATACTCACTGAAGCCATGTCTAACTTGTGTAAGCATTTATGAATACCATGAACCACTCTGTCGCGTTGTCTTATTACCTGAACTGACAATT
GAACAAAACTCTAAATCCCTCGCTTAACCATCAGATAACCTTGTTTTGTTTTGATGGAAACATAGTGTTAAAGGCACATTATTTTCCACATCCA
CCTCCTCAATGTTTCTTCCAAAACTACTCATTCCACGGCAATATACTCAGTGTTCAGGATCCACAGGTGACCACAGATGATCCTTAAACAGAAC
ATGTGACTTGAAGCCCTTTCGCCCTCCCCTCACTTCAGTTCACACAGAAATCTAGCATGGCGAAGTCTGATCTAGTAATCAAATTCCCTTGCCA
AACACCAGAAGTGTATCTAGATGTAGTTTAGTTTGCCTATCCAGAAAAATCAGAATTTTTCTCCCTTTTAAGCCCAAAGACTATTAATGCAAAT
CCAATTGAATTTGCATTTGATTACATTTCTGATGGAAACATTCATATGCAAATGAAAATTCATGCAAAGATAGCCAGATAATTCAAATAGTTGC
TGAAATATCAAGATAAAATTAGGATATTAAATGTACATCCATCCACTTTTACTGGGTTACACTAAGGTCATCAAGTGGGACAATATTCTGAAAA
GCCTCGAATAAATTTTTTAGAATATTCTAATAGGTATGTGTTCAGAAGGGAGCTGTAACATTACTTTCAGTTGTCATAGCACAAAAAAAGATGT
CAGCCACTGTTAAAGTTAGTCATATTGTTTTAAAACATTCACTTTAGCTGGGTGGTATTGGTACACACCTTTAATCCCAGCACTCTGGAGCCAG
AGTTAGGCGGACCTCTGAGTTCAAGGCCAGCCCAGTCTATAGAGTGAGTTCCAGGCCAGCCAGGACTACACAGAGAAACCCTCTTACAAAAATG
AAAAACCAAAACTAAATAAAACCATTTATTTTACATTTTGTTCCTTGCCTTCTCCTATCATTAAAAGCATAATAATAATAATAATATTTGGTAA
GACTATTTTAATGAGCCCCAAAACGGGCAAATAGTTTCTGTAAGCTGGTAGTTCCCACACACTCCCTAAGGAGTCAATGGAGCAATGGAGCAAG
TTCAGTGATGTTATCATTATCCTTTGAAAGCTCCTATGGCCTTGTGAACCGAGCAGCTGAGTCATGATCCTAACTGATGGCTAGTAAATCCAAC
TAAGAACAGATATAAGCGAAAGTTCAAGGTAAAAATGTATTCATCTAGTACCTTACATTCCAAACTCTTTCTAAAAGGAAACCTTGGAATTAGA
CAGCTATCCTTGTTAAACTTAGCAGAAGGTGGAGAGAGCAAAGGAAAAGACTTGAGGTTACAACATGCAGAATTGTTACCACCTAGTTTGGTGG
TCCATGAAATATAAAGAAAATTCTCCTGATTGTTCTTCTGTTTGATCTAATGTTTGTACATGATTTCAACTCTTTAGAATTCTAGTTTTCTCTG
CGTTGGCAAATATCACTAGCCATTTATCAGACTGAAAGCTACAGATACAGAAGACACTTCAGCATGCCACTCGGGCAAGCTGGATCTTTCCCCA
AGTCTTAAGAGATTTTACTTTGTAAATCTCTCTCAATTCCAGCAACTTTTTTCCGTCTCTACCACCCCTTCCTTAATCTTTGTCTCTTCAGGGA
TTTTGGAGATGAACATGTCCCCAGTCCCAATCCCACTGTACCCTCAGCCAACTCTGAGCAGAACCTACCCTTCCTTTTCTTCTGTCCCTGTCAT
ACTATAGTCAGAGTCATCATTAAAAAGTACAAGTGTTTTCATTCTGTAAAAGTGTCATTTTTGCTTAATATCCTTTGCTAGCTGCTCCTTCTTC
TTCTTCTTCTTCTTCTTCTTCTTCTTCTTCTTCTTCTTCTTCTTCTTCTTCTTCTCCTTCTCCTTCTCCTTCTCCTTCTCCTTCTCCTTCTCCT
TCTCCTTCTCCTTCTCCTTCTCCTTCTCCTTCTCCTTCTCNNNNNNNNNNNNNNNNNNNNTCCTCCTCCTCCTCCTCCTCCTCCTCCTCCTTCT
CCTCCTTCTCCTCCTTCTTTTTCTTCCTCCTCTTCCTCCTCTCTCTCCTCTTCCCCCTTCTTCTTGGTCCTAAAATAAAAATGAAAGACCTTAG
GAGGGAAATTTCCCCTCCATGTTCCCACTTCTGCGATCTGTTCCCCAACATCCTATATATTTTTTTCTTTCACTCTGCACTCCAAGTTAGATAC
CAAGATGTCCCTGTTCCTAGTCACCTGTGTACACACTGCTGCTCCTGCTTTGAGAGTACGTTAATTCCAAGTTTTCCTCATTAAAGTTTTCCTA
ATCTTTAGAGCTCATTTGATTCCTGTGTTACCTTTGTTTATGCCACAGTAGAGCACCACCACCATAATACCCAGTCAAATCGGAAGCAATCAGT
TTATTTTTGAGATTCTGTCATTTGAATTACTCTTTTCTTTCTTTCTTTCTTTCTTTCTTTCTTTCTTCCTTTCTTTCTTTCTTTCTTTCTCTCT
CTCTCTTTCTCTCTTTCTTTCTTTCTCTCTCTCTTTCTTTCTTTCTTTCTTTCTTTCTTTCTTCCCCTATCTTCCCCTCTCTCTCTTTCTCTCT
TTCTTTCTTTCTTCCTTCCTTTCTTTCTTTCTTTCTTTCTTTCTTTCTTTCTTTCTTTCTTTCTTTCTTTCTTCCCCTATCTTCCCCTCTCTCT
CTCTCCCTCTCTCTCTCTCTCTCTCTCTCTCTCTCTCTCTCTCTCTCTCTCTGGTGGTTTCCTGAAGGATAAGCTTAGCGTTGGTCTTCACTTA
GGAAAATAAGACTTGCTGTTTAGGCTTTCAATCATCACATGTTAAACAAGTGGGAGCTTCGATTTTTTTTCTAATCTTCTTATCTAAAATCCAC
AAGTTTATAATAGTGTGAACCAAAGGAAAAATGGGGAAGAAAAAGAAAATTCACTAACTTGCTTGAACATTATCACATCATAAACTTGCCAAGG
ATATTTCTTATAGGAAACCCTGCATGCTTCTTGTAGCAAAGAAAATATGGTAAAGATCTAGTGGCATCAGAGGGCACTGCTTTCAAGCCACAGT
AGCGTGAGTCGGCCAGGCACTGTGCAAGGCCCTTTCCCCAGCACCCATAAAGAGGAACTAACAGTTACTCTTTCAGTCCTACATTATGTATAAT
CTTTTATTTGGGATATGTAAGCCATAACAAATTTCAGAGATGTATCTAGAGATTTTAAGTACATCATGTCTTGTGACACAGATTGTTGGAATTG
GTCCACGACAATTATTTCATAAAAATAATCGCAACTTAAGAAGCTAACATCATATAGAAATCATGCTGCTGTTTTATCAGTGGGGCTCCATTAC
TTTATATTTTATTTTTTATCAGTTATGAGCTTAGTTTTATTTTAACTATACAATTCTGAGCAAGGATCAACATTGCAGTGAGAGAAAGAGAGAG
AATGTGAGTTGTTAGCCAAGTCACACTTGTGCTTGTAGAGATAGGTGTCTCTCCCTCCATTCACATCTGTCTCAGACTCTGCAGACTTTACTCT
CTTAGAAAAACATAGCAAAAGCTTTTATTTAGCAGCAGATGCATCGGTAGAGAGGGAGGTGAAGTTTAGTCTGGCTTCTCAAGAAAATTAAATT
TCCTTTTTTTGTGTGTTTCAGAACATTGTATTCAAGGGCTTGTGGAAGGGCTGGAGAGGCCTACACTATCTACTCCTCAGAGGAGATGGTCAAG
GTTTCCCTGTCACTAAAAGCTAAATGCACTTTTCTTAAGACAAAAAAGAAGTTTAATATAGACCATTAAAGTATCTGCAATGTTATGTTAGGCC
ATGTTTAGGAAATTTTTTTTAATTCGATTTTAACATTTTTTAACAAATACAATTTAAATTTTCTTTCATTTGTACATGAATTATACAGACGGAC
ATTTTAAAAACCAAATTTGCCTACTTAAAAAAAGTAAGCTCTTTTACCTGTATTGTAAAAACAAGCATTTAATCAGTGTTTGGTCATCTTCTCT
TCTGACTAATGAGCTACTTTAAGTCTTACTAGTAACAAGCCAACCTTTGCTACCAACAATGATCTAGCAGACTTGGTCACCTAGGAAGAAGACC
CTGAAAAGACCTTTCTCTCTTGCTTATGATTTCAGTTAAGAATGCAAATTGCACATCAGACTTTGAGGAATACTTTGCCAAAAGAAAACTGGAG
GAAAGAGATGGCCACGCAGTGAGCATCGAAGAGTACCTTCAGCGAAGCGACACGGCCATTATTTACCCAGAAGCCCCTGAGGAGCTGTCTCGCC
TTGGCACGCCAGAGGCCAACGGGCAAGAAGAAAATGGTAAGGCTGTAATTCATTTTTAGCCGCGATACTGACCTCTGCAGCTGTTAGAATTACA
AATGCACTACATGCCTGGGTGTTTACCTTAAAATTTGAAGAACAGCTGCAGAGGCATGGAATTTCATGAGCAAAGATTAGAACTGTAATTTTAG
GGTATTGTGAAATTTTTTTTTGTCAGCAAATTTGTGTCATTTGTACATCCAATACAGAATAAGAACAATTACAGCCCATTTCTAAGAGATAAAA
ATATCTAAATGTATTCCAATCTTTCAGAGATATTTTACTTTTGAGGAAAAAAAAAAGTTTAAGATCACCGACCCTCACCTTTTGTTCTCTGACA
CCTGAAAAAAAAAATCTCCTTCCCTCCTGTGAATTCCTGAGTTACATGTGAGTCTAGGGTTCAGGGGGACTGTCAGTCACCCAAAGGGAATTTC
TTCATTTATCAGCTGTTTAAATTCCCCCTCCCCACAAATAAGCTCAAGTAATTAATTAATTTTCGAGCGCAGTGTGAGCTGATTCGGACAAGCC
TGCCCTGGGTGTTCAAGGGGTCTGGTTGTGAGGAGGCCGGGGCGCAATGATGGCGCTGTGCAGGAGCAGATTGCAGCGATTCCTGTCATAGCAG
CCCGGGCAGCCAGCCCATTAGCTGCCATTGATTTACTGACCTTTTGGCCCGCTCCTCTCCCCTCGCCTCTCGGCTACAGACCTGCCACCTGGAA
CTCCAGATGCTTTTGCCCAACTGCTGACCTGCCCCTACTGCGACCGCGGCTACAAGCGCTTGACATCGCTGAAGGAGCACATCAAGTACCGCCA
CGAGAAGAATGAAGAGAACTTTTCCTGCCCTCTTTGTAGCTACACGTTCGCCTACCGCACCCAGCTCGAGAGGCATATGGTGACGCACAAGCCA
GGGACAGATCAGGTGGGGGTTGGTTCTAAGTGATTTCTTTCTATAATAACCCCTTAGAGAAACGATATTGCTTTGATTGGCAATCATTATTAAT
TTTTCATGGCATTTTGAAGATGCAGATCTTTTAATGGTAATAGCTTTAATTGAGGTCTAAATGATTTTTTGATGGTCTCTTCTAATATGATTTA
ATAGTCTTATGTTCACGATCGAATGAGTGTGTTAATTTCTAATTTAGAAATTTTATTTGCACTTTAACTTGACTTTAGTTTCATTTTTATGTTC
CACAAAAAGTGAATGAAATTGTAGCATTTTAATTATACATTATTAAACATCTCAGCAATTTTAATTCCATTTTTCACCTAGTTTTGCTTAGGCA
TTTTTTTTTATATAGGACCTAATACATAGCAAAAGAAGTGCATATTTCTCTCATTTGTCACGAAACACTTTTAAATTACTTAGAATACTTTACA
AGCTTACTAATTTATGAAATATGTTTTTAAGGAGATAGAAAGGCCATATAAGTTATATGCATATACACATAATAAATTGGAAAAGTGATAAATG
ATAAAAAACATTGGTTATAATTAAAATGACTCCATCTCAATATACATGCTTGTAGTTAAATGCCAAAATTGTTCTGCCTCTTTTAGCCTACTTG
TTTTAAAAATAAGAACTCTGGCTTGGAAAGGTAGGAATAGGGTCTTAAATCCTTGAAATGACTGAGTAGGGCTAATTCACAGCATCTCTTAAAT
GTTTCAGCAAAACTGCTCTTTCTATCCTCCTGGGAGAATTGACTGTGCAAATTGATATTATGATGTGTTGGGTGGTCACAGTAATGGTGTTTCA
TAAGTCACAATAACTGGTCCTACAGATACAGAATTGCCCCTAAATGAACTTTGATATTATATATTTTTGAGATGGTTAAATATGACATTCTCTA
TAAAACTCCGATTTGTCCTCATAGGCCTTATTGTCTTTGAATAAGGAAAAGAAGACACAAACTCAATTAAGCAAGAATGGCAATACTGTGTTAC
AAATTTTTACTATTTAAGGCACTATATCACATTATCACACCATTTGTTTCTGATTGGACTTGTGAGTTGAATGTTTTCCAAAGATCAGCTATAA
GTAATACGCTTGTGCAGTTACACTTTTGTAACAGTTTATCTTCTCATGCTTCTATGCACAAAAAAACTCTAGGAAAAAAAAAAAGCCTAGATAT
TTATCTAGTATAACTGCTTTAAATTTCACATGCAGAAGTGTGATTGTGTTGGCGCATTATGAATGGTAGCTTTGGTGTTGAAAAGTCCCCTCAT
TTGCTCATGGCATTTACAATTAGTAAATTAAAATGATTGTATCATATTAACAGTGGCACAACTAAAGTTTATAGTAGTCCTCTGAGGGCTGTGG
GGGAGACAAAGCAGCTGTTGCTGTTTGTTTATGCAACTCGGCAACATATGAGCACTGGTCCCTCAATGGCGCTCGGCGGGTTGGGCTCCGCTTG
ATCTTTGAAGGTTGCTGCTGTTTGAACAAACCTCCCTGTGTCCCATGTAACACCATCTGTCTCACTAGGAATGTGGGAAGATTCAGCACACCCT
AGCAGTTGTCACACCGTTTCTGGGCTGTCTTTTTGCAAAAGGCTTTTGTGTCATTGCTGCAGAAAAAGGCTGGCCTCCTACCTTGAGCATCCAG
AGTGGATGACTGTTGGGTTGTTGTTTTTTTTTTTCCCCCTTAGAATTTCAAGTCACACTGCGTTTCCCCCTTGTTCAAAAAGGTCAGCGGTGTT
GGATGGTATCTGTGCTGCCTCTAACTTGTCTTTTCAGCAAACTTTTTTTTTTCTGTTGGTTCTTGCACACTACTAAAACGGAAAATGAAATGTC
ATTGAAGTCTTTCCTACTGACTTGTGATAAAGCTGTTCAAGCAGACATAACCCAAGATGTTGTCTCTTATTTGATAATGAATCAAATCTGATAG
GCAGCACCTAAGAAAAACACAGTTAACATGGGCCAGTAGCACAGTGAATGGGATATTTAAACAGGCATACCAAATATTTATAGTCAAAGGGTTT
TTTTTTTTTTTTTGATTTTGTCTTTTATTTGGGGGTGGGGAGAGGTCTCCATTTCTTTAAATTTCAGTTTGATTTAGTTTACACTTTTACCTGG
AAACAGAAAACTTCACAAAAATTTGTTTTAAGTGTGGGTTAAAAGAAAAGAACAATTTTATAATTTTCAGGAAAGTATTTTTTTCCTCCTCAAG
CCAGCCGTGTTTTGAAAGGAAGCCTTTAGGGTTCAGATTCTGCACTAAACCAAAAGGAGCCATGAGCATCTCTACAATCATACTTTCTCATCTC
AAGGGTCAAAGAAATGCTGTAGAAAAAAGATGTCAGGCGTAATTGTGAAGCAAGGGTCCGACGTGCACGGGAAGCACTTTCTTAAAAGAGAAAA
ATTCCCATTTTATTACCAGCATTACCACTTTTATTGCAACTCTGAAAGTCTGACCAGATAAATAAGCATGAACTCAGACCTCTCAGCCTGTCTA
AAAAAGAGGTCTTGGGAAAGATTCCTAAGCAAGCTCCTTTGTGCCTCCTGAACAAAGATGACGGCCATTTTTCTCCTTTTACAGCACCAAATGC
TAACCCAAGGAGCAGGTAACCGCAAGTTCAAGTGCACGGAGTGTGGCAAGGCCTTCAAGTACAAGCACCACCTGAAAGAACACCTGAGAATTCA
CAGTGGTGAGTCACAGGGGGGTCGCTGCATTGCATTCTTTCCTACTAGAGAAGATTGTGGAGACCTTTTCAGTGTACACAGTCCCGGGTGGAGT
GTTGTGGGCTTACCCCTTTATTTCATAAGCTCTAGGAGTCTTGGAGAGTCTTATGGTATCCTATTAAATGCTTATCCTTCTTACAATCACAAGG
GCTTTGATTTGTGTTTTCTGCTAAATCTAGAGACTTTTACAAAACCGTATAACTTTGACCTGGAGATATTTGGGCTGCAATTTCAGAAAGATAT
TCATTTTTTTTTTGATACCTAAACTTACAAGAAAAAGTGGGAGAAAATTGGACAGGAACTTGCATATGCTAACCATTTCTTGACTCCTGATAGC
TATACATCATAAACCCACATCCTACCGCTGAGATTTCTGCTTTATACATAGTTTGATAGCTCTGCCAAGCATTTTATTACCAAATTGGATGCTA
TTCAAAGAACTAGTTGAATTGGTAGAATCAATGGGGTCTCCACAGAGTTGATTAAAGCCATGGCTACACTTCCCTTTAAATACTGTGACATCTT
TATTGTGCTATTGACCACACATTAATCTGACACTCTGTTTAAATAGGAGGCTGCTGATGGCCTGACAAGATTTGGGATTCTAACTGAAAAAGGC
GTAGAGAGCCTTTACCTTCCACAGCTTACATCTTGAATTATGTCCACTGGGGGTAGATAAAATCCAGAGATAGTTATGGTCATATAACTCTGAC
GTAAGCATCTAGAATGTAGAGTCCACCTACATTCGACACCTAGATTAATTTCATTTGGTTTCACAGATTCTTATCTTGGCAGACAACTTTGCAT
GTTACCTAAATGAAGAAATCTAATGCTAATTTTAATTATCCTTTCAGGTGAAAAACCTTACGAATGCCCAAACTGCAAGAAACGCTTCTCTCAT
TCTGGGTCCTACAGTTCACATATCAGCAGCAAGAAATGTATTGGTTTAATATCAGTAAATGGCCGAATGAGAAACAATATCAAGACGGGTTCTT
CCCCTAATTCTGTTTCTTCTTCTCCTACTAACTCAGCCATTACTCAGTTAAGGAACAAGTTGGAAAATGGAAAACCACTTAGCATGTCTGAGCA
GACAGGCTTACTTAAGATTAAAACAGAACCACTAGACTTCAATGACTATAAAGTTCTTATGCCAACACATGGGTTTAGTGGCAGCAGTCCCTTT
ATGAACGGTGGGCTTGGAGCCACCAGCCCTTTAGGTGTACACCCATCTGCTCAGAGTCCAATGCAGCACTTAGGTGTAGGGATGGAAGCCCCTT
TACTTGGATTTCCCACTATGAATAGTAACTTGAGTGAGGTACAAAAGGTTCTACAGATTGTGGACAATACGGTTTCTAGGCAAAAGATGGACTG
CAAGACGGAAGACATTTCAAAGTTGAAAGGTTATCACATGAAGGATCCATGTTCTCAGCCAGAAGAACAAGGGGTAACTTCTCCCAATATTCCC
CCTGTCGGTCTTCCAGTAGTGAGTCATAACGGTGCCACTAAAAGTATTATTGACTATACCTTAGAGAAAGTCAATGAAGCCAAAGCTTGCCTCC
AGAGCTTGACCACCGACTCAAGGAGACAGATCAGTAACATAAAGAAAGAGAAGTTGCGTACTTTGATAGATTTGGTCACTGATGATAAAATGAT
TGAGAACCACAGCATATCCACTCCATTTTCATGCCAGTTCTGTAAAGAAAGCTTCCCGGGCCCTATTCCCCTGCATCAGCATGAACGATACCTG
TGTAAGAGAATGAAGAGATCAAGGCAGNNNNNNNNNNNNNNNNNNNNATGAAAACATAGTCCCCAACAAAGCTGGAGTTTTTGTTGATAATAAA
GCCCTCCTCTTGTCATCTGTACTTTCCGAGAAAGGACTGACAAGCCCCATCAACCCATACAAGGACCACATGTCTGTACTGAAAGCATACTATG
CTATGAACATGGAGCCCAACTCTGATGAACTGCTGAAAATCTCCATTGCTGTGGGCCTTCCTCAGGAATTTGTGAAGGAATGGTTTGAGCAAAG
AAAAGTCTACCAGTATTCGAATTCCAGGTCACCATCACTGGAAAGGACCTCCAAGCCGTTAGCTCCCAACAGTAACCCCACCACAAAAGACTCT
TTGTTACCCAGGTCTCCTGTAAACCTATGGACTCCATCACATCGCCATCTATAGCAGAACTCCACAAACAGTGTTACGAGTTGTGATCCTCCTC
TCAGGCTAACAAAATCTTCCCATTTCACCAATATTAAAGCAGTTGATAAACTGGACCACTCGAGGAGTAATACTCCTTCTCCTTTAAATCTTTC
CTCCACATCTTCTAAAAACTCCCACAGTAGCTCGTACACTCCAAATAGCTTCTCTTCCGAGGAGCTGCAGGCTGAGCCGTTGGACCTGTCATTA
CCAAAACAAATGAGAGAACCCAAAGGTATTATAGCCACAAAGAACAAAACAAAAGCTACTAGCATAAACTTAGACCACAACAGTGTTTCTTAAT
CGTCTGAGAATTCAGATGAGCCTCTGAATTTGACTTTTATCAAGAAAGAGTTTTCAAATTCTAATAACCTGGACAATAAAAGCAACAACCCTGT
GTTCGGCATGAACCCATTTAGTGCCAAGCCTTTATACACCCCTCTTCCACCACAGAGCGCATTTCCCCCTGCCACTTTCATGCCACCAGTCCAG
ACCAGCATCCCCGGGCTACGACCATACCCAGGACTGGATCAGATGAGCTTCCTACCGCATATGGCCTATACCTACCCAACGGGAGCAGCTACCT
TTGCTGATATGCAGCAAAGGAGGAAATACCAGAGGAAACAAGGATTTCAGGTAATGAGATGAGTCACTTTGCTCTGAGGAAAGGAACATAGGAT
AACCTAGTTGTGATTCCCAGGCTGCCCAGTCCTAAATAAATTGATAAATGACCTAGAAGATACAAAAGGTGGGGGAAAAACACTCAGTTGGGGT
GGGGAAACCAGGAATATGAGAGCTTTGAAGCAAGCTAGCTAAGCCAGTGGCTGAAAGGATAAGCTTATTAAAAACTAAGGCAAGAAGATTGCTA
GTTTAAGGTTAGGCAGCATAGCAGGATCCGGTCTCCAAAACAAACAAAACAGAATAAAGAATTCTGAGAATTACGCATCGATGCTAGTAGTTGG
TCAGCTTACCGACATGCTTGAAACTGAAGTGTGTAACAGCCACACTCTCAGAAAGGAGTGTACGACTTGAAGGAATTACATATTTAAATTTTGA
TATCTATTTTGTTTTATAAAGAAGAGTTCATTTTGAAGAGAATTTAAAGAAATATTTTACATGTTACATAGGACTTCTTGCCTAATTTTCAAAT
GGTCAAAGAATTTGTAGGTGGTAGAATCTGAAAGCAGGCATTGTTTTGGCATTATTTTGTATAATCCCTGTTTGCAAAATAGGAGCCAAAGTGG
CAGTGCACGGCTGTAACAGCTGTCCAGTATTCCAATTTACAACACATCTGCCCTCTTTGTAAATGCATTTAAGCCTATTTAATCTTCTCTAGTA
TTAATTAGTGCAAATATAGTGTAAGAAAACTAAACTGTGGGCTAGCAAATGGAAATGTATGTTACCATGCAAGTTTGCACTCCTTTGGACCAAG
ACAAAAATAAATATCAAGAAATTTAATAATTTACTGATCCACATTCATTATTGCTCAATGCTGAAGGCTGTTTTAACATAGATAGAACATTGAT
AAAAAGTGTTATTAATATTCTCTATTTCAAATTAGAAAATTAAATATAAAAATATAGCTTAAGGCTTTTAGTTGGTAGACATACATTTAAATAG
ATTGTATAATAATAATATTTTGTACCTAAATATAAAATTCAATTAAAATGAAGCTTCTACAGCTACCAATCATTTTTGAAGTAAAGTACACTGG
AAAATATGGTAATCCTGATATTTAATGTTCACTCTTGGGTTTTCTCTGGTATTAAAAAAAAGTGAAAGATTTAAGAAAACTGTTTTCCCATCTA
TGGCAGAGAAGGTTTATGTTTTGTTGAAAAGTGAAACTTCCATGTTGTTTAGGAAAAAAAAATGAGAAGATTTTAGTTATAAGATAGATTGGCA
TCTAGCATTTTTGAGGATCTCTAGTTGTCAGCCTATGCTTAATTGTTTGATGTAAACACAAAGAGGAGAGACCATGTTCAACTATGTGATTATA
TCACAGGGAGACTTGCTGGATGGAGCACAAGACTACATGTCAGGCCTAGATGACATGAAAGACTCCGATTCCTGTCTGTCTCGAAAGAAGATAA
AGAAGACAGAAAGTGGCATGTATGCATGTGACTTATGTGACAAGACATTCCAGAAAAGCAGTTCCCTTCTGCGACATAAATACGAACACACAGG
TATGAGCGACTTTGACTAGCTAGCTAGAGCTCTCCGGCCTGCTGTATATTTCATAATATTTAATGAGTGCACTTGTGCGACATAAGATGTAGCA
TATGCTGACACCTTCAACAAGAAAGAAGTAATACTTTTGCATAACTCAGTAGGACTTTTGTGTTGCCTGTTTATTGGATCATTCATGTGTTTAT
GAGTGTGCTTGTCTTCTTAACTTTCAAGTTATCTGTAAAACAAATTTGCCTATCATGTCAAAGGAAAATTTAAAAGCCCAGGGTAATGAAATGG
CATGACAAGAGCTCATTTTTGAAAGTATCTTCAAATTCTTAAGCTGTGGTGAATATATATATGTGTAAAAATTATTATGATAAATCTGGTCCGT
GATATACTAGTGGACTCATCCAAACACTAAGTAAGCCATGCTTTGGGGTGACTTTCTTTGGTTTTCATAAATTGGCCACTTTTGAACCAGTTAT
AAATGTTATTTCCATAAAAGAAAGGCAAGAGACCAAATAAGCCAGTGAATCCTATAACTGAAAGACTAAAGTAATATATCCAGTTCATGTGTTA
TCTATTAATCACCAACCCTGACAAGACAACAGCCCCAGGGCTCCCTGGGACTACAGGTGGTATGTTACGTTTCCTAAAAGTGTACCGTGTAAAG
ATACTTTCCACAACTCCGGATCAGATCTGCAGCTCTGTAGTAGGGTTTCCATCAACGTTTCCTTTCTCCTTCCCATCTTCTGCTCTAACACAAG
AAACCTAGTCTTACCCTGTCCCTAATCCCTGTCCAAGTCTTCTCCATTAGAAATATGAATTTGTCTGTGGTGGGGAAAAGAAAGTATAGAGTCA
TATTCAAAATATTGTAAATGGGAGCAGGATGGACAAAGTGTAGGGAAACCTTTTATTTTAAACAAGGGCAAAAGTTAGCATTGATGCATGTGGT
GTGGAAGAGAAGAGGAGGTGTTAGATCTTAACTCTCTGCAGATAGGGTACATCTTAAAGATACCTTGGGAAACTCGGGTACTCCAAATCTTCCC
AGATCCTTCACTAGCCATGGGTTGGAGGTAGCACATGTGACCAGAATCAAGCTGAGCGCTGGGCGAAAGTGGCCCATGCTGGACCGCTAGTGCT
GAGAGCACTTTGTGAAGAGAACCCAGGTTGTAACAGCCTTGCTCCTGGTCCAACCAGTTCAGCTCCTTTCCCTCCAACCATAATATCTGTGGTT
ATTATTTTAAAAGGCCAATTAATGTTAAAACACCCCAAATTGGTTTGCAAATCTACACAATTTTTATACTTAAGCTATATATTTGAAACATCAT
TTTTTTCCTACCCTGTTCATTTATTATACTTCCTTTAATACTGAGTCTTATTTAAGTATTTACAGAGTAACAAATATTTTCATGCCTAAATTTA
CTTTAATTTTAATCAACCTCAAATATATTTTGTATTTCATTATTCAGCTACAATAGGCACATCACTGAAGGCATCTTTCCCTCTCTGATTGGTC
TTATTATATGACCATATGCAAACTCTATCATTTACTACATGGTGAGTGTAAAACTTTTATTTTAGTGAGGGCTCTCATACATAGTGACATCCAG
TCAGCATATTTATCTCCTATAAATAAGCTAGTCCATAAACTTCATAGATGTCCAGCTAGTTGAGAGACCCAAAAGTTGACAAATGCAAATGTAT
ATATATATTTATTTTTTTTAATAAAAGCACGTTATTTTAAAAGTAAAATATCTTGGTCCTTTAAGATGAGAGATCTGCTACCTAGACACATTGC
CTAGCAACAGGATGCGAGTCATTCAATGGCTTCTCTAAACTGGTGATTAGTTATCCTCCTGTTGGTGACCCAGCTTGTAGAGTTTCCAGTCTGT
TAACTTTTTGGTCTCTTGACCAAACATCTCATGACATTAATATTGTCATTGTATTAGGTTCCATCCAAGCAAAAGTCTTCATGAAATGTGTCAG
TATGATGTATATGTAAACTTAGCTTGCTTTCTTCTAACTGCGCTCCACAAGCCTTTTGCTTATATTAATATCTAATAATATTTTAAATGAGTTA
ATATGAATATTCTAAGAGAGAACTGTCTTGACTCTAAAGCTGGGCAATGAAGTTGATCTGTCATTTGGTTATTATATGTTGATTATAAATGTGT
CATTGATAAAGTATGTTTGTTATCTGTCTATAGAACTGTTTAAATTAAGAAGAAAATTAGTGTCTATTTATTCCTGGGAGTTTGTGCTCTTTTG
CCGTAATCTACTTTAAATTTATTTTCTAGGCAGATACCAAAGTCATACTGCTTAATTCTCTTACCAATGGCTTATTGCTTGCTTGTTTGCTCGG
TTTTGGTGGGGGGGAGGGAGATACCCTGGATAGTACAGAGAAAGAAATACTTTGTAAATGTTAACTTCAATTGGAATAATTGTCATAAATAATG
ACTGAAATAAAAGATCTGAACCTCTTGGTAGGCACTGTCAAGGACTCATTTTTACTCCACTAAAATTTCAACCTGATATCGATTGGTTACTTTC
TCTGTTGAGACACTGTGGTAGGTGCTTTCAAACCACCTTATTTTGAAAAGAAATAAAGATATTTCTACTTTGAAGTAGACAAAATCTAAATTTT
AAATAAATTTCTGGTCAATGTTGTTCCTAAGTTATATGGCAAAATTAAAATTAGCCCTTATTTTAAATTGAATTTATTAAGTACTTGTTAGTGA
AAGTCCTCAAGATTACTAGACTATTTAGTATAAGAGTTGTGTGGAACTCTCAGTGACTCACTGAGTTGTCCTTTGCAGGGTATCACAGTACTTT
ATCCTCCAAAGTTGTCTGTGATATATAATGTAGCCCTATTACAAGTACAGTGTCTATTACCCAATACATCCTTTGAAAGCTGGCTGCCTGTCCT
TATAGGACCCACCTCCCATGCATTCTAAAAGATCTGGAAGTTTGTCTTGGAGAATAGATGTTTTCAGTCCAGATGCTGCTCTCATTCTACAATT
TTGGAAAAATAAAACCTGTATTCATATATTCTCTCCACAAAGTGACCTAACTGGTTGACCACCTTTGATCCTGAGCTGATAAGACAGCAGCATA
AAAAAGATATCACGGGATAGTGTTGTTCATGCATCTTTTAACAACTAAAGAAACAAGAAAATTTTCATGTCTAATTTTTCTCCATTCAGATTTA
CTTCTTGCATGTATGCCTACTCCCTGATGTATGCATTTTGCTTAGAATATAGTAGATGGTTGAACCTGTATTAATGGACAAGAGTTGAGAGATT
TAACCCTAATAGGCTGCTCACTACAAAGTCCTTGTACTAAACACTTTATAAATCCTGTCTCCATTATTCTATCTATGGCCTGTCCTCAGGCTAA
GTCCCCAAATTACTTTCCTTTATCTGCAAAGACCCTGAACCTAGAAGCAGTTGACATACCCAAGACTGTACGTACTCCAGCCTGTCCTAAAAAT
GGATTTCAATGTGAGTCCAGAGCTCATCACCCTTATTGAGCACCATTCTGCCCCCATGGAACAATTGCTATCAATGCTGTACCCTTCTCCCTGA
AACATCATCATTTTAAATGTGCACTTCCAAACAAGGAAGACCAACACTATACTGTAGCAGCTTGAACATTTCCTGAAAGGTTAAAGAAAGAATA
ATACACTCCTAAGTTCTTTTCATATACTCTTGTGACTCCTATTGTTACCTACCCAAATGAAAAAAAGTACTGTGTATTTCCAGATTCCTACCAC
TCTACCAGAATATTCTGTTGTCCAGTTTCCCTCATAGAAAGCATCTTTTCCCAAACTTAAAGACTGGTTCTTCCATGCCTTAAATTAAACTTGT
TATCTTCAGCCTATTAGCTAACTTGAAGTAGGTTAATATAATTCTGTCTGCATTTCCCATTTGGTGAGTACCCACAATTTATGTTTGAATTCTT
ATCTTACAGATAAAAAGCAAAACAAAGCAAAAACTTCCTACTAAAATATTGTGCCTTCCCCTATCACTCACCACTAATTTATATCGGATGTTCC
AGTAAAATGACAACCATCAATGAAATAGCAGCTCAAACTGTCTGCAGCGTTTTCTAAGTAGTGGCAATGCCCTGGAGGCATTCAATAACATTAT
TAAAGATATTTGATTACTAATTGGGATTGTATATCACCCTATAACTGAAAACCTGAAGTGTCCAGAATGGGTTCCTAATCTTATAACATGACAC
TTAGCAAATGAAAACGGGGTTGGCCTCTACTAATGCCGGTGTTTTAATTTAATTTGCTGAAACAGGAGTTTGTAGGGGAAGTAGAATCACCTAG
CTTCATGTACGATATGATGATACCTCATGCTCCGCGCCACACATTGATTTTTAACTCTGGCGCATCAGGCTGATCTATCTCTCTATTTGTCAGT
TCTATTTTGGAAATAAAGTTTTAAGTATGACAGCTCCAAAAGGTGTTGAAGGCATTCCAGTGCTATTTGTGGCCTGGAATCAACAAGATGTGTT
TAGGCTTGGCAGTGGTTTCAAGTTCCAAACACTGGTTAGAAACTGCCTGGAATGGGAAAGGAAGCGCTAGGTTCTCTAGCTCAGGAGTATTTTA
ACACGCAACAAGTGGCTGGCATCCCATGGAAAAACTCTAGCGCGGAGGGGGGGGGGGCGAAACACCATTTGCAGCTAGTTCAGATCCTATGAAC
ACATCTTTCTAGTTTGCATTTCAGATGGTTTCTTCCCTAAGTTTTACTCTTAGAATCCAATGACTGATCAGACACTCTTAGCATCTTCTTGCCT
CCAGTCTGTTCTTTCATAACGTAGCAGTTTGCTCCTGTTTGAGACTAGGCCCACTTTCAAAGCAGCTAGCTCATGATGGACCATACAAAACCAG
TGATGATACCATTTCTATTGCTCTCACTCAAGTTCTTGCCTTAAAACAAAACAAAACAAAACAAAACAAGACAAAACAAAACAAAACAAAACAC
CGTAAGGATGAGAAGAATTGTCTGGCGGTAAAATGTTGTAGGCCACTAAAAATGTCTTGTTTCCCAGGACGTTTTTCCGTTTGAGACAGTGACA
AAGCCTCCATTTTGAGTTCCCCTTCAGCAGCCATCTGGTTAACCAATCACATTCGTTAGTAATCGGTAGAAAACCAGATTATGAGATGAAAATG
GACCTTCCTAACCGACTTGGGTTAGACCGAAGTGTGTCGATCTTTCAGACATAAGCAGACATGCTCATGCCTGAGTCGGGACACGATGACAGAA
TTCTTGGAAGAGACTTCATGCCAAGCCACTAATCACCCTGTCGTCTTGTTCTTTCTCCCAGGAAGAGACCACACCAAGTGTCAAGATTTGTAAG
AAAGCGTTCAAACACAAACACCACCTTATCGAGCACTCGAGGCTGCACTCGGGCGAGAAGCCCTATCAGTGTGACAAATGTGGCAAGCGCTTCT
CACACTCGGGCTCCTACTCGCAGCACATGAATCACAGGTACTCCTACTGCAAGCGGGAGGCGGAGGAGCGGGAAGCAGCCGAGCGCGAGGCGCG
ACAGAAAGGGCACTTGGAACCCACCGAGCTGCTGATGAACCGCGCTTACCTGCACAGCATCACCCCTCAGGGGTACTCTGACTCGGAGGAGAGG
GAGAGCATGCCGAGGGATGGCGAGAGCGAGAAGGAGCACGAGAAGGAGGGCGAGGAGGGTTATGGGAAGCTGCGGAGAAGGGACGGCGACGAGG
AGGAAGAGGAGCAAGAGGAAGAAAGTGAAAATAAAAGTATGGATACGGATCCCGAAACGATACGGGATGAGGAAGAGACTGGGGATCACTCGAT
GGACGACAGTTCAGAGGATGGGAAAATGGAAACCAAATCAGACCACGAGGAAGACAATATGGAAGATGGCATGGAATAAACTACTGCATTTTAA
GCTTCCTATTTTTTTTTTCCAGTAGTATTGTTACCTGCTTGAAAACACTGCTGTGTTAAGCTGTTCATGCACGTGCCTGACGCTTCCAGGAAGC
TGTAGAGAGGGACAGAAGGGGCAGTTCAGCCAAGACAGAGTTAGATGGAGTTGGATATTGTTTAAAAAAAAAAAACTGCATTATGCAAAAATTT
TGTACAGTGTTAAGGCCTAAAAACTGTGTGGTTCAGAGACTAATTCCTGTGTTTAATAGCATTTATGCTTTAAGCACAACTAGAAAATTCTAAG
AATTGCACTCTACGTATGTATCACTACAAACTTAAAAAAAATTATGTCTAATTTATATTAATACATTTTAAAAGGTGCCCGCACTACCATACAT
CAGTATTTTATTATTATTATTATTGTTATTCCTGTTTAATTTAATGTGCCCGCACTACAGTGCATCAGTATTACGATTCCTCAACACTTTCCTT
TCGCTATTCATGAATTTCCCATTGTTTTACAGCCTAAGTAACCACACATTTTAGGCCTCAATTTTTTATTTTTTATTTTTTTTCTGTGAAGGAA
CTTGAAGTGATGCATGTGTGAATTTAAGATACCGAAGTCTTAAAGTGACCTGGATGTGAAGGAAAAAGTAAGATGAGAAGTAAAGAAAGCCTTT
GTAAGGTGGTTTTAAAAGCCTTATGCAAACCTTTTAATCTCTGTCTCTGCAAGTGCCATCCTTGTAAAGTGTTAAGAGGGTAACGAGGGTTACC
TTTGCACCAGCTTCAGTGTTAAGCTCACCGTGTTCTTTGAAGCACCCGTGTCAGTATTAGACGACTAAACAGCAGTTCCTTAGTTTACATATGT
TTGTGCAATTATTTTCTGTACTTTTTTGTTCATTAATTTTGTCAGTATTACACCAAACTTTTTTTGCAACAAAAAAAAATTTTTTTTTGCATTC
ATTTAATTTTAGGTCAAATAACATTTTATTTATGTGGCTCATTTTATATTTCCTAATTTTATTTATTTCATACTGTAGTGTACAGTATTATAGT
TCTTCAATATATAGATATATTTTAGTAAAAAAGGAACATGACGTTGATCATTTGGGCAAATTTTACGTAAAGAGAAGAGCATTTATTGTGTTTT
GGAACATTAATTGTGAGATGGGATTTTTCAATTTTATTATTTTATTTTTGTTTTTGTTTTTCCAATTACTGGAGATTCCAATTTGGAAAACTTT
TGATACAATCTTGTGAAAACACTGTATTTTCGACTGAAAATTCCACTTTCTTCATCTTGTTTTTTAGCTAACCTCAAGAGGGACTGTTAAATAC
AATGTATGATACCATGACAAAAATCTTTCCTGAATTGTCTTTGTAAAAGTATTATTGAATTTTCAATTTGTAATTTCTTTTGAAAATGACCATG
CTCGAATAAAAATGTAGCCAAACTAAGAATGTAGTTAATGACTTCTGTACTTTTAGACAGCTTTCCTTCAATGACCATTAACATGTAACATGCT
TATGCTTATAATAATGCTAATTATGTTTTTCAATATAATTTTAGTTTAGCAATAATTTTGACTGGTACCAATAACTGTTTTTTAAAATTCCATA
CTTATGTACAGCAATTTTACAGCTTTTCTCAACTGATCCTGATTCCAGATTGTCTATTTTTTATGTGAGGTTATATTATTCAAATTTAGTCTAT
TTACTTTACAGACATTTCTACTTTTGCATTACAAGTATTTAGAGATTATGTGTTAAAAACTCACTTTTCTGTCCAAGGGGTCTTTGTGATTTAT
TCAGAAAAGTCTAATTTCAAAAAGACAGCTATTATTCAATGTTATTTATAATATGTAACTTTTTTTCTAAGAGTTGGGATAATTTATCTCACTT
TTTGAAATGCAGACTGTAGCTTATCCTTTATCTAAGATTATAAAATGGGGGGTGGGTGGGAAAACAAAGCTAAAGGCACATGCTAACAAAAATA
ACCTTCATTTTCCAAGACAGTCTTTCAGTTTTTACAAGACGACCCTAATAGTCAGTATATGAATGTATTCATAGGTTTTTACACAATGATTTTT
TTTATCAGAAACCAGATTCTAATTCCTAAATCTAATAACAAAGTCAGAATAATAATACAAAAGCAGATTACCTTATGAAATTTACAGCTCTTGA
ATATACGTAACTATAATATAGTAGCTGTCCACATATTTTTTCTACTTTAGAATCAAAAAACAAAAGCATGATTTTGCTATTGAATTTGCTAAAA
CTTTAAGTGTGACACCTCAAGTTGGCAAGAAAAACATATTTTTATTATTTAGCCATTTTCCTAAATTTCATAAACATTGTTCCTTGAAATGACA
CACACACACACACACACACACACACACACACACACACATACATGCCCCAAGAGAGAGAAAAGATGTAATGACTATACAGAGGCAATTGAGCAGA
TACTTGTAGAAAGCATATTTTCAGCTCTATCTTCAAGCCTGGTGAGGACTGGCATTAGGAGAGATGGCATAAAGCATTCTGCTCCTGTCATCGC
TATACCAGAGTTTGCATGTTTGGAAAGTTTACAGCATTCTTTCCCTATTTCTCCTTCTTTCAATGGCACAAATAAATACACTACATAGAAATTT
TCCCGATTTTAAAGGCTCTAGGCGATATCTTTATTAATTCAACCTGAAAATATCAAGCCATTAAATTTTGTCTGGGTAGAATAAATCCCTGTGG
CCTCCTTTAAAGCAATGTAGGTCTCTGCTGCCCATGGGGCATATCTGTGTCCCTACCCACAAGAGACAGGACCAACAAAGAACACATGAGCAAC
ATACTCTTTCTCCCCAGAAAGAAGAAAGCATGCATGAGAAGGAGCAAAGGGGAGCACAGAGTCTCCCTCCCCTCCTGAAATCCCTCTTTCCACC
ATTGTTTTTACAAATTACTTTTCACAGGAAGGCTCAGAATACCTGAGTCTGCAAGGATCAGGTTAACACTTGTAAATTGTGACAATCACTACAA
TGTCCTATATCTTAAGGTTTCTTTCCTTTTTAAAAATGCAATTTAGCCACTAATAGTATTGCACATTAGAGCTGCATAATCTTCCAACTCCAGG
GAAGACTAAAACATTGGACTTATCCTAGGATTCCTTTCCAAGGTCATAAACAAGAAAGCCCTCCAAATCATGACACATAATCTCCTTTTCTTAT
CGAATACTCTTCCCTTGTTCTAGTCTTAAGTATTACCCTTTACCCCTGAGGATAGGGGCTGGGAAAAGGTGATGAAATCCTGGAACTGAACACC
CCGCCCATTTTCTCAAGAGCCTTTTGTATTCTAGCAGATCTGGGAGGTTCTTTCTTTTTTGCACATGACACTGTAGGCTTAGTTCTGAGAAACT
GGGCAAGAGAGATGAGATTTCTCAGCCAGAACTAACAGCGTTCATCTCCCTTAGCATGAACTTGAGCTTGCAGTGGAGTGCCAGGGCCTGAAGG
AGGAAGGAGAGAAGGGACTATATTTGAATAGGTATTAATAAATTTATTAGATACTTTTCACAATCAGATAACTTTAAAATGTTATTTTTTATCT
TCTAAGGACATAAGCCTTAACAACATCAAAATTAGTCATAAATTTGAAGATATTCCCCAATAATAAGTTTATATATATACATATATATATAATT
TGAAATAAAAAATTGTTAGCCATGTTTTTTTGCTCCAGGATGTGTGGCCATGCCAGGCTGTGAGCCCTGGATATGCACACAAACAAGTGTGTGT
GCTCTGAAGCCCCCGCACATTGTAATAAACACAGCTGCATTTATTTGAGTATGTGTTCCCGTGTACATGTAAAAACATCCAAACAAACACACTC
AGCAGATTTATTTCATGTGCAATGGGGCAATTATTCAAATAAATATGCTCAATACAATTATTTGAGTCTCGCATTTGCATGCTCATCACTCACA
GAACTAATACTAAAAGAGGGGGAAACACCAAAGAATTTACGTGGGGGAAAATATATGTGAAAGCAATGTTATTGTAGATGTTATAAGGTAGCCA
AAGCTATGGCTCCCTTCTTAACTGAAACACAATGATTTTGTATTCAGTGGCATTTGTTTCTAACGATGAATAGTTATTTCACTCACTTGATCAT
TACATCAGAAGGACTGCAGTGTTTAGATTCATATAATAGCAAACTTTAAGGACCTGAAGCAGATAAGGATGAGACATCTCTATATTTCAAAAGC
AGAATAGTTCTTTCTACATACTTTACATCAAAGGAACACTGATAAATTATTATGGCTCACATACGTAACTATAAATTTAAAATGTTATACATAT
AGAAAAACATTCCTACATTTTAAAACATTAAAAAGTCACAGATGACTCGTGTGATCATTTTATATATTAATAAATACTACAATATATATGTGAA
CACATTACAAATCATATTGGTGAACAAAGAGGCTGTAAAGCCTCTCTTCAGTATACTGACATAACCTAATATACTAAAAGGGGAAGGGGCTTTT
AGTCACTGAATATGCATCGTGAAACAAAGATGAAGAAACTACATGGCTTGTGCCCATCACGAAAAAGATTCATACTGAAGGCTTAGCTTTGGTT
TTTATTCAATTAAATTGTCAAACTGTGCACAGTGAACTTTTTTTTTTTTTTAGAACTTGAGACATTTGTGATGTTGGCTGTTTAAATCTTTGTT
ACCTTCGCTGTGAATTGAAATTGTACATATTTAGTAAATCATGCAAACAAAACAAACTTTTTAGACAATATTTTTATTGGAGAGTTTTCTTTTC
CTGTATCCATGTTTAAAAAAAAAAGACCTCCTTTCCCAAAATAAAAAATGTCAATACTAAATTTAAAGAAGTATAAAGGAATAATTGCTTCCTT
TAGAGCAAATATTTAAATAAACATGGAGAGAATTGGCAACATGTTCTTTTGGGGCTAATAGGCTGTGTTCAATTTTTTGGGTTAAAGTTCCAGA
GGGCCTCTGTTTCACATTCAAAGATAATATATTAATCTCTGAATTAAAGAAATGCTCTTAGGTAACAGGATAAATCCCTCAGTCTTCATTTGTG
TGTGAAGTGTAGCATTCATCAGAGACATGACATTGAGACGACATGATGCTGAATATGCCACAAGCTAAAACACCTCTATTAAAACAGTCACCTA
CCACCTATAATCCAGTGTGACATATTAAAATACTCCAAAATACAGAGCATGGAGAATGAACACCATAATTCCCATTTGGCCTTGAGAAGAATAT
GACAAAGATTCTAATAATTTTTGTCAAAGAAAATGATGATTTAAAAATACATGAAGATTAAAGTGGTGACAAATGATCTTTTAAGTATTTCTAA
TTGGTCATCCTCATTCATAGCTTTGATTCCACAGAAATCACAAATCCTAAGGCCCCTGACTTGTGAGTGCGCCCCGTCAGCTTTCTTCATGCTA
CAGAATGTGGCTGTTAGGTAGGAAATGAAGGATAAGACCCCAATGTGTTAAAAATAGACTTCTTCTCCGGCAAGAGGTTTAAAATTGGCAAGGA
CAGGACTCAGAAAGAACGGCCTTCACATGAGGAAGCATTCAGAGAAAGATTGTTACCTTGTGATCTGACCATTTCCAGAACAGTCAAAAGGCTC
TCTCCCTCCNNNNNNNNNNNNNNNNNNNNCTCTCTCAAGTGTGTGTGTATACGTTTGTATGAGTGTGTTTGAGTGTATAGGAGTATGTGTGTGT
GAGTGTCTATATAATGTATATATGTATAAATGTGCATGAGTGTTTGACTATCTATGGGTGAGTATGTATATGTCTGAGAGTGAGTTTGTATGTG
TTTGTGGGCATCTATGTATCTGTGTGTCACTGAGCATGTGTGTATGTGTGTGAGTTAGTTTGAGAGTATGAGTGTGTAAGTATATGTATATTTG
TATGAGAGAGTGAGTATGTGGGTATGAGAGTGTGTGTAGCTTTACTAAGTTTACGTGTATAAAATTGGATAGATTGTATCTTTTAAAAACCAAT
ATTCAAGAGATGGAGTCTAAACAGAGGGCTTCTGAGATACTTTCTATTTGTTATCTTACATAAACTGAAAACATTTAAAATGTTGATACTGGAA
AAATACAAAGTTCTTAAGAGAGCTTCATAAAAACTGAAGAAAAAACTGAAACAATTTCTAAGGTATGTCAACAATTAAATAGAGAAAATATTAA
ATTAGCTTTTTTCCCCATCACTTAGAAAACTCAGACTTCTAAAATAGGGCCTTTCTTTAAACAATAATCATTTTTTGCCCATTCTTAAAATAGT
TGTATTATAGGGCAAATAATTACAGTATACTCCTGTCCAATTTTGTTTAGAGAAAGAGAAATATAGGCTTTAAATTACTTGAAAATGCTGATGA
ATAAAAGAATATAAATTTAGAAAGGAGACTCAAGAACACCACTAAACTCAAAGGAAGCAGGGCACTGCATTTAGTGACCACAGCAACCTGGATC
TTGGTTTTCACATGTCTTCTGAACAATATTACTTCCAACAGAAACATTTCCCAAAACTACAGGGCAGCCCAAGATCCTTGTGGGCACACGGGGA
AAGCACCAACTACTGAGTAACCCTCAACCACTTCCTGCCGTCTGAAACACTCTGTGGAGGTCTACCATCTCATTACTGAACATGCTTAGAAAGT
GTGAGGACATAGGATTCTGGTTAAAGGCAGGTCGAAAGCAAGAGGGAAAAGCAGAAGTGCAAGCCACCCTGCCCAACTTGTTTAAAAGGATATT
CCTCAAGTAAGCATGCCCAAGAGTAATCAACCTCAGTTCTAAGCACTCACCTTCCTTTTCCTCAACATCAAGAACGTTGACAATAGCCAAGACA
ATCCCCTAAAGGGGAGCTTTTGAATCACTTTAGAATAAACGGTGGTGGAATAGTCAACATAAGCTTCCAACAAGCAGAGTTCCAGCCACAAGAA
CTACTGCTACAGGTGTACCACCTTAATTTTGAAAATGATAATTAAACTAATGACTTTTTATATATTAAAAAAAAAACACATTTCCACTGGCGTT
TTCATTATAAAATCTCATGTTGTAAATTCCTTCATGACTGGGACAAAGTTATGGCATACTATCTACATTAAAATTGATTTAAAAAAGTGTTCCT
ATGAATAAAAGCTTTATGTTTGTAACATTGAGAACGATTAAAAAAAAAAAGCAATTAATCTATCACCCACACGTTATAGATGAAGAAACCGAGG
TCTGAAAATGCTTGAAAACACACAAGTGTTTGTTTTTAGCCCACAATAATCTGGAAAGCAGCTAACTGCATAAAAAGTCTAGTCCTCTGAAAGA
GAGTAACATAGCCTCTTCAAAGCATCACTTCAAACACCACAGTAAAATGTTCTTTTCAAAGGGCCCGAAGAAGCAGCAAAAGAGATGGGTTAGT
TCCTGCAAGAAGGTTTTCTTCTGGTCTATAAGAACTTTTCCATGCAAGAGCGAAATGAAAACAATCTATGTTGGAAGCCTGTGTTACTCTAAGC
TGTGACAACATCTGTACCTAGTCACTTCTTATCACGTAGAGTCTCCACTGACAGGCAGAGATGTTCTCAATTTTGAGAATACTCTTCAAAAAAT
CTGTAACTGTTGCTGCTTATAGCTGAAGAGACTGAGGTCTGCAAGTGCTTGAAAACACACAAGTGTTTGTTTCTATCTCACAATAATCTGGAAA
GCAGCTAGCTGCAGATTGCCAAAACTCTAGCCTTCTGAAACAGTGTTCTGTACTTTCTAAATACACCACAGAAAAAGGAGGAGAAAATAGGGCT
TGAGCTTTGCTCCAAGGAGCAGAAGCAGTTGGAGATTAACAAACCAGTAGCAGCAGAACTGATGGAAGAATACAAAAGAAAGTCAGAGTAGAAC
TCCTGGACACTTCCTAACCCAAGCCCTAACCTTCAGATGCTAAGGTTTAGTGAAACTAAAACCCCAGCTCATCCTGTAGCTACAGGGGACCCAG
AACCAGAGGTTGGGTCTCTCATTTTTACATAGATGATGTGATTGCTTTTCATTGTTTTAAGGGCAATTGAGTGAAAATAAAAAGCCATCCACTT
AATGGTCTAGGGATACAGTGGTATAGAAAGCAACCACACACCCCTTTAAGAATGTACATTCCAGGTGGGAAGGGAGAACAATTACAAGGTCCCA
GCGGATAGGACAGCTTAGTTAAAAATAAAAGCAAGTGAGAAAATAATGAGCAAGATGGATGTGGGGGCTAGGATTTGGGGTGTCAGGAAGAACC
TCATGGGGAGTTGAGATGAAGGATAAGAAGAAGTAAGGCATGGGAAGAGCTGGAACAAAGGTGTAGGAACAACAAGTATAGACACGAGGCACAA
GCGAGGCTGGCTTTGTGGACTAGAAAGTCCCCCTATGAGTGTAGTGTAAGAGGGGCAGAGATAAAGCCAGAAGACCACAGGCTTTTGATGAACT
TCTTAGGTTATTTGGTCCAGTGTCTTATTTGCGTAGGTGAGAAAATTGAATTTAGTAATCTCTTGAGTTTTGTACAGCGGTAGTCTGAGGATGG
GATCATTAACCCCAGTGAATTGCCTGTAACGAATTTTGCAGCCCTGTGTTTTGGAATGGGGGGAACCTCTTTTTCTCAGACGTTTGCACCTGCC
TTGTATCTCGTCACAGGCAGATCCGTCACACTGGAAAAGCAGAAATTGTTTGGCCCAAAGAAAGTTTGTATTCTGAAACTGAAGCACTAGCTAT
GGCAAATGGGATGCTGGTGGAAGCCCTTTCCCCAGTAGGCGTTTTCCAGCCAACTCCCCGTGGTTGCTAGTCTGCTCAAAACAGCTGGGCTCGA
ATCAGGCATAAGCAAGGGCCTCAGCCATTTCCCCTTCCTCTGAACTTCTTTTAAAAAAAAAAATACAAAACCTCACAGAGCTGCGAGAGAAGCC
AATAAGAAGTAAGGAACGTGCAGCAGGTGGTACCATCTGTGCCCGACTAATTCCAGCCAAAACAATCTGCTCCCTCGAGTTCCCAGTTGTGGGG
AAGGGAAGAGAATCTGACAGCGGCAGAGTCAGGGGAGGGGGTGGAGGAGGGCAGCGCTGAGTCTATTATTCCGGCGATCCGAAGGTGGGAGGAG
GCAGGTTATTGATGCCAGTGAACTATTATCTCTGTCTTTTGCTCTTTACAACCACGGAGGGAGATCTCTACAGCGCGTTCTTGCTAAGCTGGCA
ACTACATCCCACACCTGAGAGACAGACGTGCAGCTTGACTGTGAGCCTGTCACCACTGAGATGGGAGGGACAGCTTGGCAAGCGCAAGCCCACC
CCAAAGTGAAAAAGTAAACAAGAGGGCAGGGAGATGATGTTTATTTTATTCTATTTTCCTAGCAACCGAATGCCCTCTGTCTCCATGTTCATTC
TTTGAAAATTACCCACAGCTTAAACTGGGCTTAGTGTCCACATGCTTTCACCCCGGGATGGCATTTTCCGAAGCAAACTCCTTGCCATCGATGG
ATTCCTTACGAAGAACGCATCTCCCTTGGCATACTTAAAACCTGGGGTGAATAAAATGCACTAGTTCAAGGATAAATGAAGTCGCTGCTTTTCT
TTAGAGAGGAACAGAAAGGGACTGAGACAGAGCATACACACCGTTGTAGTGTCCCAGGAAAATACATCTTCTGAAAAGATTCTCTTAACCCACG
GCAAAAATTCCACCAGTGCGTTTAGGTTAAGGCACCATCCAAGGATCGTACTAGTATTTGTTAAAGGCATCTAAGGAGGGACATTTGCGTTACT
GCATTTCTTTCCGCTAGCTTACCTTTGCTACAGCTGGGGGTTGAGCTCTAAAAGATGGGTGCATTCGTCCAAGAGGGCGTTCAGAGGCTTGAAG
GGAGAGCAGCAAGAAGCATTCATACATGAAAAGCAGTCAAAGTGGGAGAAATGACTGGAAAGGAAAAAGTGCTTGATCCTAATCTCAGTTAGAT
AGAAGTTCACCTTCCCTCAGAGTGTCTGCTGAAATACTGTGGATACGTGTGAGTACAACCTTACTAGGAAACAGGTCTTTGCAGATGTAGTCAA
GATAAGCCGAGGCTACTGTAGTGCACTAATGCAATATGACACACTTACACAAGGAGGCTTTGACAAGACAGTGGCAGA
MOUSE SEQUENCE—mRNA
CGCCGAGTCCATGCGAACTGCCATCTGATCCGCTCTTATCAATGAAGCAGCCGATCATGGCGGATGGCCCCCGGTGCAAGAGGCGCAAACAAGC
CAATCCCAGGAGGAAAAACGTGGTGAACTATGACAACGTAGTGGACGCAGGCTCGGAGACAGATGAAGAGGACAAGCTTCACATTGCTGAAGAT
GATAGCCTTGCAAACCCTCTGGACCAGGACACCAGCCCAGCTAGCATGCCCAACCATGAGTCCTCCCCACACATGAGCCAAGGGCTGCTACCAA
GAGAGGAAGAAGAGGAGGAGCTAAGGGAGAGTGTTGTGGAGCACAGCTGGCACAGTGGCGAGATTCTGCAAGCCTCTGTAGCCGGTCCAGAAGA
AATGAAGGAAGATTATGATGCAATGGGGCCAGAAGCCACGATCCAGACCACAATTAACAATGGTACAGTTAAGAATGCAAATTGCACATCAGAC
TTTGAGGAATACTTTGCCAAAAGAAAACTGGAGGAAAGAGATGGCCACGCAGTGAGCATCGAAGAGTACCTTCAGCGAAGCGACACGGCCATTA
TTTACCCAGAAGCCCCTGAGGAGCTGTCTCGCCTTGGCACGCCAGAGGCCAACGGGCAAGAAGAAAATGACCTGCCACCTGGAACTCCAGATGC
TTTTGCCCAACTGCTGACCTGCCCCTACTGCGACCGCGGCTACAAGCGCTTGACATCGCTGAAGGAGCACATCAAGTACCGCCACGAGAAGAAT
GAAGAGAACTTTTCCTGCCCTCTTTGTAGCTACACGTTCGCCTACCGCACCCAGCTCGAGAGGCATATGGTGACGCACAAGCCAGGGACAGATC
AGCACCAAATGCTAACCCAAGGAGCAGGTAACCGCAAGTTCAAGTGCACGGAGTGTGGCAAGGCCTTCAAGTACAAGCACCACCTGAAAGAACA
CCTGAGAATTCACAGTGGTGAAAAACCTTACGAATGCCCAAACTGCAAGAAACGCTTCTCTCATTCTGGGTCCTACAGTTCACATATCAGCAGC
AAGAAATGTATTGGTTTAATATCAGTAAATGGCCGAATGAGAAACAATATCAAGACGGGTTCTTCCCCTAATTCTGTTTCTTCTTCTCCTACTA
ACTCAGCCATTACTCAGTTAAGGAACAAGTTGGAAAATGGAAAACCACTTAGCATGTCTGAGCAGACAGGCTTACTTAAGATTAAAACAGAACC
ACTAGACTTGATGACTATAAAGTTCTTATGGCAACACATCGGTTTAGTCGCAGCAGTCCCTTTATGAACGGTAAGGCTTGGAGCCACCAGCCCT
TTAGGTGTACACCCATCTGCTCAGAGTCCAATGCAGCACTTAGGTGTAGGGATGGAAGCCCCTTTACTTGGATTTCCCACTATGAATAGTAACT
TGAGTGAGGTACAAAAGGTTCTACAGATTGTGGACAATACGGTTTCTAGGCAAAAGATGGACTGCAAGACGGAAGACATTTCAAAGTTGAAAGG
TTATCACATGAAGGATCCATGTTCTCAGCCAGAAGAACAAGGGGTAACTTCTCCCAATATTCCCCCTGTCGGTCTTCCAGTAGTGAGTCATAAC
GGTGCCACTAAAAGTATTATTGACTATACCTTAGAGAAAGTCAATGAAGCCAAAGCTTGCCTCCAGAGCTTGACCACCGACTCAAGGAGACAGA
TCAGTAACATAAAGAAAGAGAAGTTGCGTACTTTGATAGATTTGGTCACTGATGATAAAATGATTGAGAACCACAGCATATCCACTCCATTTTC
ATGCCAGTTCTGTAAAGAAAGCTTCCCGGGCCCTATTCCCCTGCATCAGCATGAACGATACCTGTTGAAGATGAATGAAGAGATCAAGGCAGTC
CTGCAACCTCATGAAAACATAGTCCCCAACAAAGCTGGAGTTTTTGTTGATAATAAAGCCCTCCTCTTGTCATCTGTACTTTCCGAGAAAGGAC
TGACAAGCCCATCAACCCATACAAGGACCACATGTCTGTACTGAAAGCATAACTATGCTATGAACATGGAGCCCAACTCTGATGAACTGCTGAA
AATCTCCATTGCTGTGGGCCTTCCTCAGGAATTTGTGAAGGAATGGTTTGAGCAAAGAAAAGTCTACCAGTATTCGAATTCCAGGTCACCATCA
CTGGAAAGGACCTCCAAGCCGTTAGCTCCCAACAGTAACCCCACCACAAAAGACTCTTTGTTACCCAGGTCTCCTGTAAAACCTATGGACTCCA
TCACATCGCCATCTATAGCAGAACTCCACAACAGTGTTACGAGTTGTGATCCTCCTCTCAGGCTAACAAAATCTTCCCATTTCACCAATATTAA
AGCAGTTGATAAACTGGACCACTCGAGGAGTAATACTCCTTCTCCTTTAAATCTTTCCTCCACATCTTCTAAAAACTCCCACAGTAGCTCGTAC
ACTCCAAATAGCTTCTCTTCCGAGGAGCTGCAGGCTGAGCCGTTGGACCTGTCATTACCAAAACAAATGAGAGAACCCAAAGGTATTATAGCCA
CAAAGAACAAAACAAAAGCTACTAGCATAAACTTAGACCACAACAGTGTTTCTTCATCGTCTGAGAATTCAGATGAGCCTCTGAATTTGACTTT
TATCAAGAAAGAGTTTTCAAATTCTAATAACCTGGACAATAAAAGCAACAACCCTGTGTTCGGCATGAACCCATTTAGTGCCAAGCCTTTATAC
ACCCCTCTTCCACCACAGAGCGCATTTCCCCCTGCCACTTTCATGCCACCAGTCCAGACAAGCATCCCCGGGCTACGACCATACCCAGGACTGG
ATCAGATGAGCTTCCTACCGCATATGGCCTATACCTACCCAACGGGAGCAGCTACCTTTGCTGATATGCAGCAAAGGAGGAAATACCAGAGGAA
ACAAGGATTTCAGGGAGACTTGCTGGATGGAGCACAAGACTACATGTCAGGCCTAGATGACATGACAGACTCCGATTCCTGTCTGTCTCGAAAG
AAGATAAAGAAGACAGAAAGTGGCATGTATGCATGTGACTTATGTGACAAGACATTCCAGAAAAGCAGTTCCCTTCTGCGACATAAATACGAAC
ACACAGGAAAGAGACCACACCAGTGTCAGATTTGTAAGAAAGCGTTCAAACACAAACACCACCTTATCGAGCACTCGAGGCTGCACTCGGGCGA
GAAGCCCTATCAGTGTGACAAATGTGGCAAGCGCTTCTCACACTCGGGCTCCTACTCGCAGCACATGAATCACAGGTACTCCTACTGCAAGCGG
GAGGCGGAGGAGCGGGAAGCAGCCGAGCGCGAGGCGCGAGAGAAAGGGCACTTGGGACCCACCGAGCTGCTGATGAACCGGGCTTACCTGCAGA
GCATCACCCCTCAGGGGTACTCTGACTCGGAGGAGAGGGAGAGCATGCCGAGGGATGGCGAGAGCGAGAAGGAGCACGAGAAGGAGGGCGAGGA
GGGTTATGGGAAGCTGCGGAGAAGGGACGGCGACGAGGAGGAAGAGGAGGAAGAGGAAGAAAGTGAAAATAAAAGTATGGATACGGATCCCGAA
ACGATACGGGATGAGGAAGAGACTGGGGATCACTCGATGGACGACAGTTCAGAGGATGGGAAAATGGAAACCAAATCAGACCACGAGGAAGACA
ATATGGAAGATGGCATGGGATAACTACTGCATTTTAAAGCTTCCTATTTTTTTTTTCAAGTAGTATTGTTACCTGCTTGAAAACACTGCTGTGT
TAAGCTGTTCATGCACGTGCCTGACGCTTCCAGGAAGCTGTAGAGAGGGACAAAAAGGGGCACTTCAGCCAAGTCTGAGTT
MOUSE SEQUENCE—CODING
ATGAAGCAGCCGATCATGGCGGATGGCCCCCGGTGCAAGAGGCGCAAACAAGCCAATCCCAGGAGGAAAAACGTGGTGAACTATGACAACGTAG
TGGACGCAGGCTCGGAGACAGATGAAGAGGACAAGCTTCACATTGCTGAAGATGATAGCCTTGCAAACCCTCTGGACCAGGACACCAGCCCAGC
TAGCATGCCCAACCATGAGTCCTCCCCACACATGAGCCAAGGGCTGCTACCAAGAGAGGAAGAAGAGGAGGAGCTAAGGGAGAGTGTTGTGGAG
CACAGCTGGCACAGTGGCGAGATTCTGCAAGCCTCTGTAGCCGGTCCAGAAGAAATGAAGGAAGATTATGATGCAATGGGGCCAGAAGCCACGA
TCCAGACCACAATTAACAATGGTACAGTTAAGAATGCAAATTGCACATCAGACTTTGAGGAATACTTTGCCAAAAGAAAACTGGAGGAAAGAGA
TGGCCACGCAGTGAGCATCGAAGAGTACCTTCAGCGAAGCGACACGGCCATTATTTACCCAGAAGCCCCTGAGGAGCTGTCTCGCCTTGGCACG
CCAGAGGCCAACGGGCAAGAAGAAAATGACCTGCCACCTGGAACTCCAGATGCTTTTGCCCAACTGCTGACCTGCCCCTACTGCGACCGCGGCT
ACAAGCGCTTGACATCGCTGAAGGAGCACATCAAGTACCGCCACGAGAAGAATGAAGAGAACTTTTCCTGCCCTCTTTGTAGCTACACGTTCGC
CTACCGCACCCAGCTCGAGAGGCATATGGTGACGCACAAGCCAGGGACAGATCAGCACCAAATGCTAACCCAAGGAGCAGGTAACCGCAAGTTC
AAGTGCACGGAGTGTGGCAAGGCCTTCAAGTACAAGCACCACCTGAAAGAACACCTGAGAATTCACAGTGGTGAAAAACCTTACGAATGCCCAA
ACTGCAAGAAACGCTTCTCTCATTCTGGGTCCTACAGTTCACATATCAGCAGCAAGAAATGTATTGGTTTAATATCAGTAAATGGCCGAATGAG
AAACAATATCAAGACGGGTTCTTCCCCTAATTCTGTTTCTTCTTCTCCTACTAACTCAGCCATTACTCAGTTAAGGAACAAGTTGGAAAATGGA
AAACCACTTAGCATGTCTGAGCAGACAGGCTTACTTAAGATTAAAACAGAACCACTAGACTTCAATGACTATAAAGTTCTTATGGCAACACATG
GGTTTAGTGGCAGCAGTCCCTTTATGAACGGTGGGCTTGGAGCCACCAGCCCTTTAGGTGTACACCCATCTGCTCAGAGTCCAATGCAGCACTT
AGGTGTAGGGATGGAAGCCCCTTTACTTGGATTTCCCACTATGAATAGTAACTTGAGTGAGGTACAAAAGGTTCTACAGATTGTGGACAATACG
GTTTCTAGGCAAAAGATGGACTGCAAGACGGAAGACATTTCAAAGTTGAAAGGTTATCACATGAAGGATCCATGTTCTCAGCCAGAAGAACAAG
GGGTAACTTCTCCCAATATTCCCCCTGTCGGTCTTCCAGTAGTGAGTCATAACGGTGCCACTAAAAGTATTATTGACTATACCTTAGAGAAAGT
CAATGAAGCCAAAGCTTGCCTCCAGAGCTTGACCACCGACTCAAGGAGACAGATCAGTAACATAAAGAAAGAGAAGTTGCGTACTTTGATAGAT
TTGGTCACTGATGATAAAATGATTCAGAACCACAGCATATCCACTCCATTTTCATGCCAGTTCTGTAAAGAAAGCTTCCCGGGCCCTATTCCCC
TGCATCAGCATGAACGATACCTGTGTAAGATGAATGAAGAGATCAAGGCAGTCCTGCAACCTCATGAAAACATAGTCCCCAACAAAGCTGGAGT
TTTTGTTGATAATAAAGCCCTCCTCTTGTCATCTGTACTTTCCGAGAAAGGACTGACAAGCCCCATCAACCCATACAAGGACCACATGTCTGTA
CTGAAAGCATACTATGCTATGAACATGGAGCCCAACTCTGATGAACTGCTGAAAATCTCCATTGCTGTGGGCCTTCCTCAGGAATTTGTGAAGG
AATGGTTTGAGCAAAGAAAAGTCTACCAGTATTCGAATTCCAGGTCACCATCACTGGAAAGGACCTCCAAGCCGTTAGCTCCCAACAGTAACCC
CACCACAAAAGACTCTTTGTTACCCAGGTCTCCTGTAAAACCTATGGACTCCATCACATCGCCATCTATAGCAGAACTCCACAACAGTGTTACG
AGTTGTGATCCTCCTCTCAGGCTAACAAAATCTTCCCATTTCACCAATATTAAAGCAGTTGATAAACTGGACCACTCGAGGAGTAATACTCCTT
CTCCTTTAAATCTTTCCTCCACATCTTCTAAAAACTCCCACAGTAGCTCGTACACTCCAAATAGCTTCTCTTCCGAGGAGCTGCAGGCTGAGCC
GTTGGACCTGTCATTACCAAAACAAATGAGAGAACCCAAAGGTATTATAGCCACAAAGAACAAAACAAAAGCTACTAGCATAAACTTAGACCAC
AACAGTGTTTCTTCATCGTCTGAGAATTCAGATGAGCCTCTGAATTTGACTTTTATCAAGAAAGAGTTTTCAAATTCTAATAACCTGGACAATA
AAAGCAACAACCCTGTGTTCGGCATGAACCCATTTAGTGCCAAGCCTTTATACACCCCTCTTCCACCACAGAGCGCATTTCCCCCTGCAACTTT
CATGCCACCAGTCCAGACCAGCATCCCCCGGCTACGACCATACCCACGACTGGATAAGATGAGCTTCCTACCGCATATGGCCTATACCTACCCA
ACGGGAGCAGCTACCTTTGCTGATATGCAGCAAAGGAGGAAATACCAGAGGAAACAAGGATTTCAGGGAGACTTGCTGGATGGAGCACAAGACT
ACATGTCAGGCCTAGATGACATGACAGACTCCGATTCCTGTCTGTCTCGAAAGAAGATAAAGAAGACAGAAAGTGGCATGTATGCATGTGACTT
ATGTGACAAGACATTCCAGAAAAGCAGTTCCCTTCTGCGACATAAATACGAACACACAGGAAAGAGACCACACCAGTGTCAGATTTGTAAGAAA
GCGTTCAAACACAACACCACCTTATCGAGCACTCGAGGCTGCACTCGGGCGAGAAGCCCTATCAGTGTGAGAATGTGGCAAAACCGCTTCTCAC
ACTCGGGCTCCTACTCGCAGCACATGAATCACAGGTACTCCTACTGCAAGCGGGAGCCAAAGGAGCGGGAAGCAGCCGAGCGCCAGGCGCGAGA
GAAAGGGCACTTGGGACCCACCGAGCTGCTGATGAACCGGGCTTACCTGCAGAGCATCACCCCTCAGGGGTACTCTGACTCGCAGGAGAGGGAG
AGCATGCCGAGGGATGGCGAGAGCGAGAAGGAGCACGAGAAGGAGGGCGAGGAGGGTTATGGGAAGCTGCGGAGAAGGGACGCCGACGAGGAGG
AAGAGGAGGAAGAGGAAGAAAGTGAAAATAAAAGTATGGATACGGATCCCGAAACGATACGGGATGAGGAAGAGACTGGGGATCACTCGATGGA
CGACAGTTCAGAGGATGGGAAAATGGAAACCAAATCAGACCACGAGGAAGACAATATGGAAGATGGCATGGGATAA
HUMAN SEQUENCE—GENOMIC
CTGCTGACACAACCCTACTCCCAAATTTGCAGGCAGGAGAGTGAAGCAATGTCTGTACTCTATACATAGTCTTACTTTTTTCTTTTCTTTTTTA
AAGTAGCTGTTTCAGCATTCAACTGGGGGCAAGAGGGGTTGGGGGGTGGAAAAAGATAGTGCAGGCAAACTTTCTCAGTGCTATTCCATCCCTC
TATGTCTCCCCACTCCTTCACACCTCTGAAGTTATGGCAAGACTAGGTAAAGAGTTTTGAACAATATTGAGACCCTCTTATATTTACCTTGAAC
AAATAGGATCTTTGACACTTCTTGACCACACTCAGCTGCCTGCAAGAGTGCTTCCTCCTATAGCAAGTCATATCGGGAGAATTCGCTGAAGAAA
GGTTTTTTCCTTACAATTGCAAATGAAAGTATAAAATAGATCACCAAATTTTAAAGTGTGCATATGTCCTCATCTCAGCTGTTGACATCATATT
TATAATCATGAGCACGCCAGGTTTCCTACAACTTAAGAAATTCTATGACCATATTACTTTTCCAACTTTATCAGTAAACATTGATTTCTGTATA
ATGCAGAAACCAGTTAAATAATTTCTGGCCGGGTGCAGTGGCTCATGCCTGTAATCCCAGCACTTTGGGAGGCCGAGGTGGGTGGATCACCTGA
GGTCAGGAATTCGAGACCGGCCTGGCCAACATGGTGAAACTCCGTCTCTACTAAAAATACAAAAAATTAGCTGGGTGTGGTGGCAGGTGCCTGT
AATCCCAGCTACTCGGGAGGCTGAGGCAGGAGAATCACTTGAACCTGGGAGGCGGAGCTGGCAGTGAGCCAAGACTGTGCCACTCCACTCCAGC
CTGGGTGACAGAGCGAGACTCCGTCTCAAAAAAAATAAATAAAAAATAAAAATAAAATAAAATAAACAACACCTAAAAGACATGTTACATCCAC
TCTGGTTTTCCATAATTTAACTTCATGTATTTTTCAGGTAATTCATGATAGTAGGAATTACTATTTATTGATACACCTGCTCTGTGCCAGACAT
TTTGTATACAGTCATATTTTCTTTACCACAACCAGCTTGAATCTAAGTACTCCTTTACCAATTTTTTTTTTTTTTTTTTTTTTTTTTGAGACAG
GGTCTCGCTCTGTTGCCCGGGCTGGAGTGCATGGAGTGCAGTGGCACCATCTCGGCTCACTGCAACCTCTGCCTCCTGGGTTCAAGCAATTCTC
TTCCTCAGCCTCCCGAGTAGCTAGGATTATAGGCATGCACCACCACGCCGGGCTGGCTGATTTTTGTATTTTTAGTAGAGATGGGGTTTCACCA
TGCTGGCCAGGATGGTCTTGAACTCTCGACCTGGTGATCCACCCTCCTCGGTCTTCCAAAGTGCTGGGATTACAGGCGTGAGCCACTGCACCCA
GCTCCTTTGCCTACTTTAAAAATAAGGTTCACATGGGTAAAATAACTTGTCCTCAATACACAATTAGTATTTACCAAGCAGGGAGCCACATCCA
GTTTATCTAATTCCAAAACCTGTATCTTTGTCACTATAATATGCTAACATTGAGGATCTAGTGTCAAATATACTAATGTCCAGTTTCCAGTCAA
CAAGTGCTAACAGAAGTTGTATAAGAAATAAAAACTATTCCACGACATCTCTGGACCATGATGTCTCCTTCATCACTAACTTGTCCTGTGCCTG
GGCCCAATGTTGGAGCCAGTTGAGCTAGCAAGAACCTCCCATGGGAGCTTTTCCTAAGTCGGGTGCCTGGGAGAAGAGACTGGACCAGAAAATA
CACTTTATCTGGGAAGCCAGAGGCACCAAAGCCAGAGAAACTTTGGCTCAAGGACTTGAGCCAATATTGTAACTGGAAAAATGAGCCTAAGTGT
TAGGTCCAAAAGGCAAAATAAGCCATACGTGGAAAAACAAGGGCCAATCACAGCATCCAGGAGGCCTGGAAATGGGTCACAATGTGCAGAGCGG
GTGGGCACAAGTGGGTCAGAAACACATCTGGGAAAGGTAAAAAGATGTTGGATGTCACATGAATATGGTGTTGGCCCTGGGGGTGGACGTGGCA
GATTCAAAAGCACACCAAGAACCAGATCGTTGAATTGTGGGGAGTCACTGACTCTCCCTAAGCCACAATTTTCTCAGCTAATAAAATGGACCCA
GTTGTAACCCCTTCAGATCTGCTATCAGAATTAAATGATACGTGAAAGGATTTAGCATAGTGCCAAGCCTAGAAAAGCAGGGGTTTTTTTTCCT
TAGCTATTATTACTTTATATTTGTGATTGCCATGGAGATGATAATAAATTTTGAGAGTAAAGTTTTTACATTTAAGTACCTGAAAGAACCCTGA
AAAAAAAAATTCACCCTTAGAATGGTACAAAATTAAAACTGAGTACAGAACAAAGTTTATAGTTTGAGCCTTTTAACAGAATAAAGAGTATTAA
AGAAGACCTTGAAGCAAGCATGAAGTAATTTCCAGGTCACTAGACAATTTGCAGGTGCAAACCATTTCTAGTCCCATTGTCAGAGGGAGAAAAA
AGGTACAGGTCACACCCAGTCCCCTACGCTGATGTCCAAATGTCTGGTTTGGTAGTTTTGACCTAAAAGGAGCATGGAATGGATGCTTTCAAAA
TAGAAACAATTAACAGACCCTTTAAGAACTACAGATATAAGTGTTTTGAGAAGTCTTAAAGAAAACAAGAACAACCAAAAACTATCAGTGTTAG
TATTACATCACGATAGTTTTTCCCCCTGGCATTTGCATTGATTTTTCAGTTTTACATCAAAAAGAAAATCTGGCCTTGTGGACATTTAAGAGAC
TAGGGAATTTCATTTTCCACTTAATACACTGATTTTCTTCACCACTCTAGGTACTTGAAATTAGCGCAAACCCCAGGACAGCTACAATTCCAAA
AGGGAATGCTCAGTTCATTATGAATGGTCTTAAAGCAAACAGTGCTCTGACAGCCAAATTCCTGGTCTTCAAATTTGTAAGTTTGCCCTTTTTT
TCACAAGTCTTACTCAAGACCGCTTCTGAGCTTTTGCACATTTAACCCTTACACTAAAAGACAATATGTTACTAAAGAGCATGGGAAACTTCAA
GTTCATACATTTGGACTAAATTCCTTTTCAAAAGAAGTCTAATATTTATAACCAAATTATGACCATATTGAATACACTAAATTAACTGCTGAAC
TAACAGAAAGGAAATACTGTATTTATTTTCACTTCTGTGGCTACGCATCCTTTGAACAGTTCGAAAAAATCATAAACCAGCCATAAATCCTAAA
ATGCTTTTCATTTTCTTTCTTCACGTAATAACACAGCACTGATTTTTCAATAAAAAATATACTGTAAATAGTTGTTATTTTTAATAATCAAAAT
TAATTTGTACCATCTTTAGTTGATCTGTATATTTGTTTAACCTTTCATTATAATAATAAAGCTACAATATAGTGAAGTTTTATGCATTACTTCA
TTTAATTCTCAATAAAATCCCAAGGGATAGCATTATTAGTGTACCCACATTGGAGATTAAAAATAAAACAAGCTTATAGGAGACTGCTTATAAT
CACACAGGTAGTCATTTGCAGGGCTAGTATTCAAAATAGATATTAATACTTTGGACTTTAACTGGCACAGGAGAATAAAAATATCAGGGATACA
CTTACTTAATCATTTTTAATAATTTAAATCAGCCCATATGTTAGTAAGTAAAAGATAAAAAATGATCCTAAATACCAGGCCTGAGTTATTTGTC
AATTGCTGCAATTTTCCACTTTTAGACTTGAATTAATAGACATAACTAAAAATTTAAATGACAAAATTCCAATAAGAAAACATAGTGCATAATT
AGTGCACATCTCTGGATTTGGGGAGGAGAGTGACTTGATTTTACATAGACTATTGTTTTTCCTAATTAATTGTAGTGTGCAACATATTTGTATG
TGAATGCCTCATAAATGGTTTACAAATTGTTAAGAAGTTTATGGATATCTAGAAAGAATATTACAGCTCAATAAATAAACTTTTACAAGTGTAG
AAGAGAAATTTTGCATTAAAATGTGACTTTGTTTCTTCATCCTGAGAGAATGAATTTTTAAAAAACACATTTAAAATCAAGCATTAGATATGCA
AATATGTGTTCAGATCAGAGATACTAAAATTATTCAAATGATTAATTATTCATCATAAAATGTCAATTAATAATTATGAAGCAGAAAAGGCATC
TTTCTAATACATCTTTATTTTTCTTGGCCTAAAAAGGGCTAAATATACATGTTCAATTGACTTCTATCAAGTTTTTAAAATTCAACAAAGTAAA
CAAATTGTAAACAAGTGAAAACTCTGAATTTTAATGAGAACAACAAATATGCCCAAAGTGAAGGGAAGAGCAAAAAATAATAATAAAGAAAACA
AAACAAAAACAGTTATGTTCATGACATATGCCGCCTCATGCTCACTCACCTGCTAGGTAACGGACAGTAACCTAGCCTAGTCAAGAAAATAACC
CAACAACCCAAGTTGTGAATTTGCTTAGTAATGTGCAAACTACGTGACATGATGATGTTTCTCTCTGAATTGATCAATCTTTGTTGGGGTTCAG
GAGGAGTGCTTTGGGAATTTTTTACCCTTTGCAAATAGTCATCAGAAGAAAAGCGCAGTTTAAGTTTCATAAATTACCTATTGTAAATGTCTAA
AGTCTGTTAACAAATTAAGTACTACTAAAAATATGGCTGTAAACAAACTTTTTCACATTGCCTTAATTCTAAAGAATGCCCTATGCCAAGCATT
ATGCATTTTTTCCAGGAAGACTATATCTATTATATCTTGTCTTTTAAAATTATCAGTCTGAGCTGATGTGATAATATAGAAGAAATAACCATTA
TGGTCACTGAAATATTGATTTATTTTATTTTTATTATTATTATTTTACTTTTCACTGACACCTGGTGAACTTGTCGTCTCTGAGACCCGGGGAG
TTATTTTAGTCAGGGTGTCCCTGCCCTCCTGCTGGTAGATTAATTACACATTAAACGTTCTGTTTTCAAAACGAAATGATAAATCTGGACACAT
AAAAGAGGCACAAAAAGAATCACAGCGAGGGAGAAGAGTGGATGTTAAGAAAGAAAACAAGAAATTGCAAAAGGGAGATATCAGATAGAAAGGA
GAACGTAGGAAATGCAACTGGGAAACAATTTCAAGAAAATAAGGATAAAGGGCAGGGAGGGCTGAAAGGAGGGCCAGAGGGAGGAAAAGCAGGA
GGGAGATTTTTGGTTTTCAATTCCCTGGTGAGGCTGCTGGCGTCCGCGCCCTCAACGCCCCAGCCTTCCAAGTTGAGGCGAAACCCACCTCCTC
GGGCCTGCGTCGCATCCAACTTACTCCAAGTTTTCTGGTGACTCCTACAGGGCCCCTTTCCGTCCCCCCCCCCGCTCCCCGCCCCCCAAGCCTT
AGTGAGATTGACAGTCGCGTGGAGGAGGCTTTTCCAGGCACAGCCTTTCTCATGGTAATCAGCCCCCGGCATCACCTCCCGGCCGCCGCTGCGC
CCGCTCGGAGCGCGGCTCGCGGACCCCGGGGACCGGAAACAGGGCGGCGGGCGGGGAGCAGGCGAGCGAGCCAGGTCAGCTGACCGGGCGGCGG
GCGGGGGTCGCAGGATCCCGGGCGGGGACAGGTCTGGGGGAGGGCGGTCTGGTGGGAGGGGGCCGAACACCGCCCCCCCCCGCGACGCGCTCCT
CCGCCTGTGCAGCGGGGCCCTTCCTGGAGATCAAATGCAGGGACCGAAGCTGGCGGCGGGGGTTGGGGCGGGGTGTGTGGGGGGGGGAGTGGCA
GAGCGCGCGGAAAATTGGGGACAACAACCAGACCTGTTTAGCATCAATAAATTAAGATGCTCATTTGGGTCCTGAAGTTTGCTCGTTCCTGTCA
GTAGAGACGTTGGCAAAAGCCCTGAAGCCGAGATTCAGCCTTTACTTCAAAAAAATAAAATAAAATCAACCCCTCAGCTGGCCGCCCTGCTGGG
GCCAGGTGACGTGCGAGATTCCACAGCCTGCGGGTCTCGCAGCAGGGATGTTAGGGACGGTCTGAGCTTCTCTGGTCCTGTTTTGGTTTTGTTT
GAAAGGGCTTCTCAGAGAGATGCTCCTGATTTTTTTTTTTTTAATAGACTTTCCCCTCCTATCCTAGGCTCAACTCCGAATGGATTGGATTGCG
AGTCTGCACGTGAGAAAACCGTTTGGCTTGGCTTGGACCCCTGCCGCCCCCCACCTCCTCCACACACACCCAGTCCAGGGGTCCCCTTTATCAC
CCTTTGCTTGCAACTCCAAAAGAAGTTGCCCACCTCCTGAGTCACAACACAAGGTCGAATAATTCCTCTAGATGAAAGATCAGTTTCATTTCAA
AACGAGAATAGGTTCCTTTTTTTATTTTCTCCACATGGTACAAAATAAACAGAATTTGCTTTAAAAAAAATCATTAGCTCTGGCCAAATTCGAA
TTCCTTCTACAGCCTGTGTGTTCAAGGGCACAAACATTGTATCTCTTCGGGCATCTGGGCCGCCAAGCTGAGCGGGGGGAGGGGGGAGCGGCTG
TGAAAGTTGAGGAAATTGACAGACTGACAGGTGAATGGGGGGACAGGCACCACGTCCTGGCTGGAGGGAATTGGAAAACAAAAGTGCATCCTTC
TCACCCTGCCTGGGTGGAGGGAATCCTGAGGGAGGTAGTTTGTTTTGCCCATGGAAACCCCAAAGAATTATTTTTCCCCCTTCAATGTTGATTG
CAATATTATTTCACATTTCCGTGTATCTGTTGTTTTAATCTCTTTCGTCTGCGCTAATCTCGAAGTGGATTATTAATGGGGAAGACAGAGAAGG
AGAGATTCATTTGCAGGACTTATTTTGCTGTGTGAGGAGCCGAAACCCAGCAGCCCCCAGTCCTCTCTCGACTTTTCTCTGCGTCCTGTGCTCC
TGCATCCTCCTGATCCCACGATCCCCATTGTCCCCGAACTGGCCCTCGCACTGAGGGAGTGTTCGGAACAAATTTATATTCCGGCTTCCTCCCG
ACCCCATCACCTCTCCCACGTTGGGAACTCTCTCGGCCAAATGCCTGGGGAGGGGCGTGCCAGTCAGCAACCAGGGGCAAGGCAACCTAGCCGC
AGCGCTCACCCCTCGCGGAGACCCTTCCCGTGCCACCATCCAAATATCTACCTTCATGGGAACCTGGAAGACTGCTTTTTCACCCCAAATCAAA
TACTGGCTAGTCGGAACTCCCAGTGATAAATGTTGGGGACTGCCTCCTGCCACTTGAGCAATTCCTGAGTCCAGGGTAATCAGATGATTAGGTC
TTTTTAATGGAAATACTTAGGGTTTTTTTTTTTTTTTTTAAGTCAGATCCTAAGTAACCTGACATTTAATATACATAATAAAGACAAGCGTTTT
GTTAGCTATTCGATTGGATCCTTTGACCTGAGGTTATGCTTGTGTGTGTGTCTGGTCTGTAAGCCTCCAATGGCTACCTGGGTGCCTGAAGAGG
AGTCTTGCCTGTAATAACTCTATGCAAAGGGCTCCTTCTTCCTTAGGAGTGGTGGAAGCCTGGCTGTAAAGCAAACGTCTAGAGAAAGCTGGAT
TCCAATCTTTTAAATTAAAAAAGAGGAAAAAAAGGAAGAGGAAGAAAAATGGACATGAACAGTGGTGCTAAATCTCAGAAGAGGAACTTGATAA
GTCACACATATATTTAGTATAGATATATAATAATGAAAGAAATGTAAAAATATAAATTAAGAATGTGCCTGACCCATGTTGAAAATCTTTGATT
TTATTTATTAATAATTTTTAAGCTGAAGATCCTGAATGCCTTTCTTCTATAGACACTTTATCTGTGATGGGGTGGGGGGAGAGAGTTAATTTAT
CCAGCAGCTATTTTCATTATAATAAATACTTAAATTAGGAAAGAGGTGAGTAACCAACTGTGAAAATCTACTTTCTTGCATCCTAGAACAAGAT
CTAGCTGTGGTCGTGCTAGGTAGCTATTAACCACCCCCACCCCACTCCCCCCCCCCGCAAAAAAAAATACAAAATACAAAGACGTATACCTCTG
GGAGTGTGGATGTGGCAACCCATCTGGTGGCTTGTCCCTCCTTTGAGTGCAATTGCCAGTGACTCTTGCATTCAAAGTCTTGCTTTAGGCACAC
ATTCAAAGCACAGTTCAAAGCAGTTTATCTTAATATAGTGTTGGATCAGGGTATTATTTGTTTCAGATTTCATCAGACTGGAAACAGGAGGGTG
TAACATATTTAACAATATTAAAGCTTAAAAATTGGTGTGAGGCAGCTCTCTGGTCCTCTTAACACCTCCACCTTTACCTTTCCGAAGAATCCTA
AATGCTGTGAGGCTAGAAGTTTATTTGTGCTCTGAGGTTCTCAAGCACTTTCACTCTTTAGGAAAAAAAAAAAAGTCTCACAGTTATTTAAGAA
ATTGAGCCTGAACCATCCGAGGATAGGAGATTTCTTCCTTTTCCGATAAATGCCTGTGCTCAGCATCCTCAAATTTGGCTGTCTTTATTTACTT
CTATATTAGTTCCAATTATGTGCAGTGTAAACCAGCTTTTTGATTAACAAAACAGCAGAGCATTGGTTAAGAACACTTAAACTTCAAAAAAATT
CCCCTGGGAAGTGGGCTCTAAATTGGTTAATAATGTGCAGTACTTGCTAAATTGCTGACTTCCAGATGTGGAAAATATCTTTCTTGTTTAGGAC
CTATGTAACCTGATTGGATTACGACAGAAGCGTCACGTTGCAAGCTTTTGAGTGATTATTTAATTAACCATACAGTAGAAACCTGTATTCTCCA
GGAAATCCCTTCCATATTTGCATAACCAATCCCTTCAGAGCAAAGGTGGAGTCTTTTCTTTTTAATCTTACTTTAATTGTAATATCAAAAAATA
TATACTCCAAAACTTAGACCCCATGCCGTTTAATATCATGCTCTCCCTTCCCTGCTAAGTTTCTCTATGGCCTTTCTCGTTTCTCCTCCTGCCT
CCCTACACACTCTCCCTTACTTTGTAGTGAGGTCTCCCCGAGGTGTAGAGAGATTCAGACATCGGCCAACCGAGTGTTCTATTTTAATTTACTT
AGAGACCCTTTATTAAAATGCCAAACTACTTTTTAATATTGGGATCCAGTCCAGAAATTCATCATGCACACACCCTAATACACATGCCCTAAGA
TGCAGCTCCCATGCAGCATTTTTTTTTCTGGCTCTGGTACCTAAAAAGAAAAAAATAACAATAAGAGAAAGGGCAGAGAACTTTGTTCCAGAAG
CTGTACTGAGATACCTACACAATTTGATGTGCATCTCAAATCTGGTCATTAGAGATATCTGTATAAGAAGAGACTATCTGGATTGAGGACCCGG
GATCTTTCCCTTTAACTTTCGCCCCTTGGAGTTCTCCAGTTCTGTGAATGGTGTGCACCGTTTTCCGCCCTGTACTCTGTAGGATTTAGTGATG
AGGATAATGATGCCAAAGGCTTGACGGGCTGCCCATTCATTAGGGCATCTGATTAACATAGTAAGGAATGTTTATTTTCAGTACATAAAATTGT
AGAACTTTGGGAGCATCGTCTGAATTGTCAGAGCTGAACAGCGAGGAGCTTAGTGAGACATGAGAATATAGAGAGGTACCTAATGTGTGGGAAG
CTGAGCTAGTTGATATTCTATGAGAAGTTCGTCGTGTCCATCTCATGCTGTAGGTGTAGGGAAAGTTGTTCAGGTGCTGCTCACTAGTCCTTTG
AGATGCAGGTGCCAGTGGTCTGATCTCTAGTTGATGATGAGAGAGCATCTACTTTCACATACTGAGTGTCTTGAAGTGATNNNNNNNNNNNNNN
NNNNNNGAGGCGAAGGCGAAAGGGAGGGAGAGGAGGAAGGGAGGGAGGTGGAATTTCATTTCTTCCACTAAAGCGTTTGCGGAGACTTCAAGGT
ATAATCTATCCCAGATCCTTTCCCAGAGAGAAACTTGGCGATCACGTTTTCACATGATGCTCACCCTCAGGCCGCTTCAATTATCCCTCCCCAC
AAAGATAGGTGGCGCGTGTTTCAGGGTCTCTCGTCTCTCTCCTACAGAAAAGAAAAAGAAAAAAATGTCATTAGAAGAGGCGTAACACGTCAGT
CCGTCCCCAGGTTTGTGTTTCCTGGAGTGGCCGAAAGAGATCAGTTCTAACCTGCTCTGCAGGAATAACGGTCCTGCCTCCCGACACTCTTGGC
GAGGTTTTTGTACAGTTTGCTCCGGGAGCTGTTTCTTCGCTTCCACCTTTTTCTCCCCCACACTTCGCCGCTTCTTCATGCTTTTTCTTCTCAC
CATTTCTGGCCAAAACTACAAACAAGACTTCGCAGGTAGGTTTTTTTTCCTCCCCTTTTCTCTCTTTTTATCCCTTTTTGGTGTGCTCGTCCTC
CATCCTCCTTTTCTAATTTTCTCATTTTGAGTGGGGATGTGAGTCTGAAGTGAGAAGGGGTGTCCGTGGGTAGGAATTTCAGGTGGGTTTAGCT
CCTTTATGGCAAGCTTTTCTCAAGAGTGGCTTCTTCGCTTTCTCTTACACTCACACTCTCTCTCCTTGCTTTTCTCTCCTTTTTCCCTCCCCCA
AATAATTTCCCCTGTACCTGTTCATTCAGGTAACCAAACGTCTCCTTTGCTATCCAGCCCAGGAAAAGTTTTATTGAGAATTATATGGCTGGAA
TAAAAACTCATCAAGGGGAGAGGTCCTGCTCTATTTTCCCATCTAGTTCCAATTCATTATATTCAGCAATTAGGACTTGGCAAATACCTGTTAA
TAATTACAAAACTTGGTCAGTTACAGACCTACAAATTCGGGGGCAAGTCTTTCTCTTAGACATGGCAGTTCATCCTCTCCCACCGAGAAGGCTG
TTTTCTAAGCAGAAAGTTGTCTGTTTTTGTCTGCTTAGCGAGTTTCATTTCTTTATTTCATTCTTTGTGTGTGTATGTGTGTGAAGTAACACCT
CACTTCTGTGATTATAAATAAAGAAGTGAAAGTCCTCATGGAACTTGAGTCGTTTACCAAAGTGGAGGGGGGGGCACTACAGCTGGGAAGAAAA
CTGCTGTCAGGATATCAGGGTTTTAAATTAGGATAACTTTTAAAATTGTATTTTTAGTGGCAAAGTAATATAGACATGGATTTAATAGAAGACA
CATACACACATATATCCCATTCCATACTTTGGCACTGAAAAGTCGAGAGAGAGATAGCAGGAGTTCCCAGGAACAGTCATGAGCCCGGCTAACA
TGAATGTGTTTATCTCCCACCTACTGATATATTTTACATGCTCCCCCTGTAATGATGAAGGCAAGATTACTAACTCCAGACTTAGGAGTAACAG
GCAAAGATAAAGTTTACAGCACAAATGCCCTGTCTCTCTCTCTCACACACACACATGCCCATAAAACTAGCAACTTACTTTATTAAACATGTAT
TTATATGCAGTCCTGTGTCTCAGGATGAATATAGACAGGCCAGCTGTTTCATCCCAGACACTTGTGTGACCATTTCTTTATTTCAGGATTTCCC
CTAACAATTTCAAATCAATAGGACAAGGAAAGAAATGATAAAATATGTCAAACTTAAAAGTCTCCTACTATGTATGCCTAGGTAAATTTAAGGT
GCTTTCTAAAATATCCACTTCCCCTTCTCTCTGCCCCCCTCCCTTCACCCCTCTCAGCAAATGTGTGGAAACTGATACTCACTTTCCAGGTTTT
CTGCAAAGCTTCTAGCACAGGAGACAATAGGTGTGGGGCGTGGGGACTGAGTGTGCCCGGCAGAGAAAGGGTTAATGGCCCTTGTGTTACAGTT
TGCATCTGTTACATCTTTTATAGCCCCCTGTTTAAACTAATCCTCCAGGCACAGCATCTTCCCAACCTCCACCGAGTTTTCAGGATTCTCCTCC
TCTCCTGCCTGCCTCCGAGGTCCCCCTTCTCCAAGTTTTGACAGTTTCAACTGAAAAAATGCAGCGCCTCTCTCGATTTTTAGGTACAAAAGTT
TCGAAGCCAGAAAGGATACGATCATTTAGTAGAGCTTAAAAGTATTGCCGTAGTGGTTGGACTTTCTCTTTCCTTTCTTAAGGGAAAAAAAAAA
TTCTACTGGAACTATTTAAGGGAACCAGTGTGGGGGAGCATGTGTGTGAGTGACGTGGGGGGAAATACTTAAGTAAATATTGTCAAAGTGGCGA
GGGGGGGTGGGGTAAAGGACAGTGTCCAAAGAGGCTTATAATTATATTTATCATTGCGGCGTAAAACGGGAAATCTTAATTTGGGGGCGAGGGT
GGGGGGAGGAAGAGACAGTGCCTCGATAAACTCCAAAGGGGAGGGGAGGGGAACGCTGGGCGAGTGGGCTTCCTATAATTACTATTATCAATTT
GCCCGAGTGTGCTTCATCCAGGGGCTGTTTCGCGTTGTTTGTTTGTTTGTTTCCTCTGGGAATTGGCGGGTGGGGGGATGAGAAAAGATGAGAA
CGAAAAGAAAACCTCGCCTCACCCCAAGTTCCCGAGCCGGGGGCTGAGGACTTTTCGAAGGAAAGGGTCCGCCTAGCCCTGAGTCAAGCGGTCT
TACTGTACCGCCGTGTGCATTCCCTCATACGGTCAGGAGTTATGACTCATTTTGAAGATGTAATTCTTGTCTCTCTGATCCCCTCGCGGGTGCA
ACACACCAAACAGTAACAAACACAAAGCGCCTCGGGGCCAAGGCTGGGGGTGGGGGGCGGGAAGAGCGGCGGCCGAAGTTTCGCTTTGGGGTTG
GGGGGCGCGGAGAGGTGCTCGCCGGGGCGGTGGCGTGGGCCCTGGGCGGCCCGCGCGCGTTTCAATGGGCGCGCGATGCCCACATTGTCGCTGT
GTTTGGTTGCTAGATCGAGCCTGCGTGCTGCCGAAGCAGGGCGCCGAGTCCATGCGAACTGCCATCTGATCCGCTCTTATCAATCAAGCAGCCG
ATCATGGCGGATGGCCCCCGGTGCAAGAGGCGCAAACAAGCCAATCCCAGGAGGAAAAACGGTAAGAAGCAGCCCGAACCAAACTTTTCCGGGC
CACTACGCGGCTCGAGACCCAGGGAGAAGGAGGGGGAAAGGGAAAAGGAACCGAGGCGGCGGCGGCGGCGGCGCCCAGGGCTCTAGGGCCAGCC
TCCCTCCGCGGCCGGCGCCGGAGCCCGGCGCGCGCGGCGGCTGGACCGGGGGGTGTTGGGGAAGTGCCGCGCAAAGTGAGCGTCAGGCACCCGG
GGGTCCGAGCGCGGGGGTCCGGCCGGGGCCCCGCGCCCCCAGCCCCGCTGCCCCCCTCGAGCCCCTCCCTCCGCCCTCCCTCCGCCTCCGCCTC
CCGCTCCGCTCGCGCCCGGCCCGCCGGGTCTCCGGCCGGGCTTCGGGAGCGTCCGCCTGGGGCAGGGCGGGGGCCCAGGCGCTGGATCGCGCAG
GATCCCGGGGCGGGGGCGGGGGTGGCAGACAGACTGCGAAGGCAGGAGAGGGGAACCGGGTCTGCGCCTCAGCCAGGCTTAAAGTTTCTTGCCG
GAGTCGCCCGGCCGCTTCTCGGTGCGCCAAGTAACGGTCCGCGGGCAGCGGGGCCCGGCCCATCGCGCCCAGACCCCGGGGTGGGAGGGAGTCG
GCCGGCCCCGCGCGGGGTCCGGTGGGGTCGATGATGGCTCGGGGACGGATGACTCCTCTCCAGCAGGCTCTGGCCCCCAGGCCGGGGACTGAAG
GAGTCGCAGGGCGCAGCGAGACTTTTCCCGGCAGTTTCTACATGGTCTGGAGGAGCCCGTTATCCAGGCTGAGTCAGGAAGGAGGAAGGAGAAA
GGAGATGGGGGTTCGAGGGGCACACGCAGATCAGAGGTGGGATATTAAAGGGCGGGGGTGAGAGTTTGGTAGGTCCGTTTTGGAGACAGCCAGA
GAAGGGACGCGGGGGGACACAGAGAAACAGAGAGAGAGGAAGAGAGTGAGAGAGACACCCCCTAGGTATTACCCCAAACAAAATCGTGGGAATA
AAAGAGAAAAGTGATTGGCAAGAGCGCGGAAGGCGCCTGGGGGAGGGGAGAGGGCTGGGGACTAGCGAGGGTGCCGGGGGGGGGTGGGCGAGCT
GACGGAGGATAACTGAGTTTAAAAAGAAAGAAACCAACCCCCGAAAACAAACAAGCGAACGAGCGCCAGCCCAGACTCGGAAAGGAAACTTCTC
GCTCCCCTCGCTCTCAGCCCGATCACCCGGCGCGCGGCCGCGTAGACGCTCCCGAGGCAGGCGCTGGGACGCAGCGCCCACCCGGCCTCCCGGC
CTCCGCAGCCCCAGCCCGCGCCTCGCGGGGACCGCCCGGGCCCACTTGGAGCAACTTTCCTCTAGAGAAGACAATTGTGCGCGCCCCCGGCCTG
CCCCAGAGCTGCGCGGCGCGGGGAGGGCGCGGCTGGGGGCGCAGTGTGAGGTGCTGGAAGGAGCAAAACACAAAGACGAGAGAATTTTGATTAG
AGACAAATGATGCTTTTCTCTCTCTCTCTCTCTCTCTCTCTAGGTTTTGTGTGTGTGTACGCGTGTGTGTGCGTGTGTGTGTCTTTTGCTTGTT
GTGGTGGAGGTCTCGAGCCATATGGGGGAGTGATAGAGGAATTTTAGTCAGAAACTGTTCGTTACGTTATCCTGCCTTTCCCCTTTGTGCTGTC
TTTGATCTATTTGCTTGGCTTAGTATTACAGAAAAACAAGTACAATAATATATTCCGAGCTCCTTCCCCAAGATTCAGTTCAGGCTGCTTCTTT
TTAAGTCTCTCCCTCTCTCTTTCTCCCTCTCTCTCTCTCTTAATTTAATTTTTTTTTCACTCTGGTGTCTGGCATTATTTAAGGTGGTCCTTAC
ACAGGAGACCTCCCCTTCCCCATCTCTGAGTAATTTAGTAGACACTAAAATTACACCTAATGTCCCCCTCTCCTCTCTTGTGTTTCCTCTGTTC
CTTTTATTAAATGTTGGTTATTGATTTTCTAAAATCAATGTCTGTTGTGGGGCTTTTTTCCCCCTCTTACAAGTATCTAAAGCACGTTGCTGAT
CAACTGGATAACAAAAGCTTTGTGTCAAGGAGGCTTTTTTCCCTCTTCTGTTAAACACTCATTTTTTTTTTTTGAGAGAAAAAGAGAATGAGAT
TTAAAACAAGGCTCCCACAAGTGTTCTTTTGCAGGGAAGACTATTTAAAGTGTGCATTTCTTCTTCCCTTCCTCCTTCCACCTCCTTCGGGATT
TTGCCACCGTTGACCAGCCTGGCCCTAGCAGTGAGGTCAGGCCAGAGCCCAGGTCTAAGGAAATAGAGGGATCCTGGCTTCCTGGGAACCCCTA
GGAGACCTGGCAGAGAGGGAGGCCCGTTGGGCGCTTCGCAGGTGGAATGGAGTGCCAGAGATGTCCGCAGTCATGGACACTGCAAGTATTTCCC
CTCCACCCAGCCACTAACTTGGGAGAAGCCTCCACATTCAGAAGTGGAAAGTTGAATTGCACATTTTAAGGCATTTAGCCCAGATCCTGCACAC
TTAATATCAAGCCTGTCCATTGCTTACAGCTTCTCTAACTTTGACTGTTACCTGTCTTTTTTTCCAAATAAGCTGTGGGAAAACATGCTGTATA
CTGGTTATTGTAAGTGTTTTTCACTGGGTTTAATCTGACCGAGCAATGTTCTGGTTTTGAGAAGCAGGTAGAAGAGTACTACCACCCCACCCCT
TTTTCATTTTCTTTCTCATCAAACACATATCCAATGCCTTTCCCTTTGGGAATTGCTAGGATTTATCTGAACTGAGTGGATTTTTCTTCTTTCC
TGGATTCATGGAAAAGATGTCACTTTTGAGCTTGTGTCTTTGCTGATAACACTAAATATGATGATGCTTATCAAAATCATATAGAAATTCCTGG
AAAGAAATAATGGCAATAATAATCGTATAAGTAGAAGCTCGAAGGCAAAAAAAGATGGTCATCCAGGAACCTAGAATGGCACTTTATATAATTT
TAATGAAGTCAACAGTGTATATATAGACTAAGGCGACAAGGAGATAAAACGTGTAAAGCAGTGTGTGTGTTTTAAAGGCTGGTCAAGAACGTGA
GTTAGAAGACAATGCTATGTACATTTAATAAAAAGCAAAGGAGAAGGAGGCAGTTGAAGAAAAATAAATGTACAAAGAGAGAAACAAGTATCTG
AAATACAACCTTCCAGATTCTCAGGCTAGCAAGATGCCTGGCAGAGGCAGTGCCAGGGCCAAGTTAACCCATAGCGGGCAGTCAGTCTCCTTTC
CCCCACATGGAAAGGATGAAATCTCTTCCCAGAAAATAAGATGTGCAGGAGGAAAGAGGGAGTGGGGTGAGGGGGAAGGACGCAAAAAGCGAGT
TGCCCGCAGACAAGAATCTGTGTCGGTTTCAAGAAAGTTCAGTCAGATCATCCTCAGTCGCCTGACTCACTTTGTAACACTTTCACTCACGCTG
GAGAGGAGGGGGAAAACCCAGCCCCCCTTTTTCTTTCCCCTCATTATACCCCACTATCTCCACACAGCCTTGGAGTCAGAAATGAGCACTCGGA
GCGGGAGATGCCCTGCTGCTGCTTGCCACCGGTGCGGCCCGTTTGTAACTTGCAAAGTTTGTTGCTTTTGCCCCTGATTCGGGCAGCCGGTCCT
GGGATGCTCCTGCTTCCCTCCTGCCTCCCACGGAGCCCGGGAAGAGGGTCTGCCTCCCCATCCCGCCACCTTCCAGCATCAGCCTCTGAAAAAT
CTCACAGAGACATGCACGTTGTAGCAAAAATCAAATCCGGAAACTGCTTGTTTCAGAGAAAGAAATGAAGTTGTCTTTTAAAGAAAAACTGAAT
TAGGAGGAGAGAAAAGGGAAATAGGAGAAGAAAGGAAAAGTTAAATTTGATTTTTCTCCAGAGTTTCCACTAAAGGGTTGGGGACAGTGTGAAG
GAGAAGGGGAGCTTTTTACAAATGCCTTTGGTCTCTGAACTTCAGTGGCAAAGAACAGGGATCAAGTTGAATGTTCTCAGGGCTTTGGATCCTA
GAGGAGAAACAATCAGAAGAGCAGAAATGGTTATCCCTGTTTAAAATAAGCCCTCACTCTTTACCACTTCCTTAAAGGAGTGGAGGTGCTGGTA
GTGATGGTTAGAGGCAATGAGGGACGGAGAAGTTGCTCCCGTTTCAGAGATGCTTAAATGAAAAGGAAAGAAAATGCAGTCAACCCCTTCTCCA
GGAGGTGCCTCCTAGCTCTCCTCCCTGAGAGGTGAAGTTGGGATGGGGCAACGAGAGTCATACACACTTAGACAAGGAAGTTTCCTTCGGATCA
CTGTCAGTCCAGACTTGGTTATCTTTGCAAAGTGTGGAAATCTTTGGCAAGTAGCTTTCTTCGTAAAGTTGATGAGCTTCTAGGGAGCCTGTTT
TGCTGACTTTCAAAGCACTGGGGCACGTTGTGTGGCAGGTACCACTTCTGAGGGCGCTCCAAAGATATCCATCTCCATCCTTTTTTCTCTGTGG
AGATCTTCTGCAAGTTTTGTCACGCTGCACACACACAAGGCTGGGGGCTATGTATCTAGGCTGATCTATTTGTTTTATTTTGGTCTGGAAAAAC
TAAGCCAATTGGGGTAGAAACATGCTTTCCTTCGTAGCAGAGCCAGTAGGCTGCTGGTGTCCATAGAGTGACAGTCCACCACGACTAAGGGTGG
GCTGAGGATTTTAAACTTTACATTGTTTCTCTGTTACCAGATACAAATAAATTCACGTCTTCCACCATTTGTTTTCAAATAGGGTAAAACCAAG
ATTAAAGTTCCCTGCTCAACTGCTATGTCATAGGTTTCAGTGTTTCCCTTCCTTCTTAATTTGCTTAAAGAAAATTCCAAGAGGTTATTAAAGA
CCTTGATGCCATATTAAGAATATTTCCTGGGAAAAATGTATGTCTACCCTGAAGGTAGGAAAGGAGGGCGTTGCTAGCCTCTAGCAGTGCCGCG
TTTATTCTAAGATGTGGGAGATTCTTTTCCTTGCAACAGTTTTTGTCATCTGCATTCTTCCAAGGCTTTTAAGGTGCATTTTCTTCTGTGTGAA
AGGAAATTCTTTGTCCTTTTCCTCTCAGCACCGTGGCTTCCCAAGGTAGACACTATTTTGTGCCTGTCACAGAGAGAGGGAGTGCAGGTTTGCA
ATGCTCACAGACATTGATTGTCTGCCCTAATAATGTTTCATTTACATGTTTATAACGTCAATGGTGCTGGAATGTCCACTGTACCATTCATTCC
CGCATTCCCACAAGGGGGCAATTGTCTGAATGGCCAAGTCAGACACCTTTTTGATTGCTCTTTGGTTGTCTTTTCAGAGCAAAGAGATAAAGGA
GGAAAATCTGTGATGCAGAAACACTAGTTGAAAATATACAGAATTAAATGTCACCACAAAAGCAGATGTTAACATAAGCCCAAATATGCTTTTT
AGCCAAGATGTGAAGGTTGAAAAAAATAATTCAGAGCAGAGGGAAGGATGATTTAAACCAATAAATATAGCCCTATTCCCCCTCTTTACTTCTT
TTCTGTCTTTAGCAATCAGAAGATGAAATGTAATTTTCCTTTTCATTTTTAAGCCTTGAAACATCCAGGCACCTCCTCATTATTTGTATGTTTG
CTGTGATTTGTGAATTTTGTATATATTTACATAGCTCTGTTTATGCCAACAGCATCAGCTTACCACTTGGAAAATCTATTGAATGACTATTTGG
GCTGTGGGGAGGGTAAACTTTTAAAAAGTAAGATCCAAGTATTTCTTCATCAAGCAGTTTTTAAAAGGAAAACGATAATAATCAGTAGGCTCCA
TGGAAGCCTTTGCCTTAATAGCTATGTGCCAAATACTTTTATCTTGTGTGACAGTCATGTCAGAGTGAAATCTAATCAGGAAAGTGTAACTAGT
AGTTACAAAGTAAATAAAGGATTTCATTTTAAGGTGTTCTGTAGTGTTTTTATGCTGTGTATGTGCATGTGTGTGTTTTGTCTTGTGTTTCCTC
TGAGGAGGTAAGAAGAAAAGCCATCCATGCGTGTATCTTTTGGCTTTTCTCTTCTTTCCGTTCTCTCCTACATATTATGCATATATGTAGCCCA
CATGAATACACCTGTTTAAAAGAAAATCACAGTTCAGATGGTGCCTTGCTGGTTCATTTAGGAAACAACAGTTATTAGAAGTTGGTCTGATTTC
GCGTGTGGTCCATGAAGACAGAAAATACCAACTCTTGTGTCATCTGGTGGGAGGCAAATGGGGGAAAAACTTTTATAATTTTGTAGTAAATTAG
AGAGGCGAAAAAAACTGCCAGTCTGTGAAAAGATTTTTGCCTCTCCGGGAAGACACACACACACCCAGTAGCTGCACCGTTAGCTGAACCCCTG
ACACGGCGGTCTTTTGAATGCCTTAGGTATGATTTCACTTTCGCTCACATCAATGCTGACCTGAATGCCTTTCATATCTGATCCGCTGTGTCTC
TCCCAGTAAACATCCCCTGAGGGGAGAACAAAGAAAGGAGCTCTTCTTCTTCTTAATCACAATTCAGCCCTTTCACCGCCGGCAGCCTCCATTT
GCATTCTGCCACAGAAAGCCAAGATGAAAAGAAAGAGAGAGAGAAAGAGAGAGAGAGAGAGAGAGAGAGAGAGGCACAGGGTAGTAGCTGAGTA
AAATAACTTAGGTGTTTGTGGCTGTTTGTTGGGCTTCCCCTAGTTACCAGCCTTATATAGCTGATGCTCCACCAGGCCCTGTCCCTCCTGACAG
GCATATAGTGTTACAAGATGGCATGTCTGTACTATTCATGGCCACCCGCTCAACACTTGAGGTAAAAAGTGTCAGGAGGGGATCGAATTCTCCC
CCAGAAGCCTGTTAGATTTGGGGTCAGGTGTGTGGACTTCCCCCCCTTAAGTGCTGCCTACGATTTGGCTTTAATAAGTCTGATTGTTTACCAG
CGGACCAGAGAGGTGAGGGCCCTGGGGGTGGGAGGGACGGGCGAGCTGGAGACTGGGCCCCTGGTCTGGTTTCAGTGACGGGTCCTCACACCCA
GCCTGAACACACTGATAAATCCTCAGGAAAACTGGAAAAGACTTTCCCATTTTTCCCCCTGACTTTTTATGATAATTCATATTTTCAAATGAAA
GGGGAAAAAAGCCAAGAAGAAACTCTCAAATGAGCCAAAAAGGAAAAAAAAAATTCTCTGAAAAGAATTTTAGGGGGAAAAGTTGTGCTATTTT
AAAAAGCTTTGAGGGCTCTAAAGTGCCACGTTTTTCACACCTTCTCCATACTTTTTTTTCAGATTTTTTTAGTATGTTTAACATTTGTGAGATG
ACAGCCTTTTTGTATCTCAAACTGGATGCTTCAGGATTTGTAGGGGAGGGGTTGGTGGTATTCACGTTCTCTTGTGAGAGAAAGTTTGTGCCTA
AAGGCCTCCTTCCTGGGAACTGTTTAAACCCTCGCAGGCAGACCTGTCAGGTTTTACTGTGTTATTTGCCACCATTTTCAACATGCAAAAGTAA
CATGCAGTTAATGGTATTGGAATGAGACCACAGCAAGAGCACAAACCTCTCGTGACAACTTTTCAGCGACATTATCTTGCTCCTAGACTCTCTT
CTCTTTTTCTTCCCCTTTCTGAAACCACTTTTGAGATACCAATAATTTTCTCCCAACAATTAGATTAAGGATACAATCCGCTCACACACATGCA
CCACACACACACACATTCACACACACAAACACACACACAAACTTTGGAAGTGCAATCTCTTCCAACAGCAGAGTGAATGATTTGAAACGGGGGC
TTTTCCAATAACCCTTGTGGAAGATTTCACTTATAAGACCAAAACTAACGGATATATTAGAAGTTGTGCGAACATGTGACACAGATTCCTGATT
TTTTTTTCTTTTTCCCAGAAGATTTACAGAATTTACAAAAAGAGATTATCCCTACACCTTCCACCCCTCCTATCCACCCCCATGACCCAAGCAA
TGAAGGATTTAGGCAGATTTACTTCAGTAAACACCTTTGGACACTTGCGGGGAAAGTGGGAGTCTGTTTGCAAAAGGAGGCTTTTGTTAAGTGA
GGTACGGGGATAATGCTTTACCAGGCAGCTGTGATATTAACCTACCCAGCAATGCGGGAACACTCTTTAACTCTTTGAGTGCCTGGCCCCTTGC
GCTCAGCAGATAAAATCCTGGCTCTCCAAAGACACTCAGAATTCTGGCTCTAGCAGTTTATTATTCAGACAAGTCTTATAGGCATGAAAACTGT
TCAAGCTTTTAAAACAATGTGGTTTAATTAGATGAGGTGGACTTACATTGCTCATAAGCCCTCCAAATGGTGGTTTATAATTGGCAAGGGAAAA
AAAAAAGATAGAAAAAAGGAATCAAGGCAGCACATTAAAAATTCCAGGAAATATCTTTAGAAAAGCTATTTTCATTTCTAGCTTTTAAAATATT
AGACTACGGTAGCTAGAGGGAATGAAGCAGCTGGGGCCTCTCTCCTTCCTATAAAAGACACCAGAAGTTTGTGATGAAGAAAGGGGCTTCAACT
TGAAAAGCTATGAGAGGCCAGTTCGGACAGTGATATGATCTCAGCCTTCGCATCTTTTCAAGGGGGAAGTACTGCCTATCTGTCCAAACCTGGG
AGGTCATGTGAACCTCAGAGTCAGGGCCTCAGGCTGGCCGTTACCAACTTGAGACTTTCCAAAGCAAGCAGAGAGGCATTTATGAGGAAGGAGT
TAATGGTTATTTTTTGCTGGGCACAAGAATAACTTTACAGGTGTTGCATTTGGGCTTAAATAAGATTGCCTTATTTAGTTTCGAAAAAGAGTGT
AGGTGTCAACATTCGAGCAGCCACAAATGTGTGAGAAAGTAAAGGGTATGAGACATGACGGAATTAAAGGTGTTTGCTATGTTGTTACACCGTG
TGTCAGGCTGCAAGCTCCCTTTGTCACAGCCTCCTGGTGATTTGACAGCGCAGATCTACTTCCCTTCTAGGTGAGACAGCATTTTTAAACTTGA
GTTATAAATATGGTACACCCTTCGAGTTGCACCCTGCCTCCCAGATCCGTGAATCCTATTTGAAAGCATCTTGCCCTGAGACACAATGAACAAA
CGCCCGTATACAAGGAGGCTGTGGGCTCGGCGGCCCGGGCAGCGCCAGCATCACAGTAGCTGGCATTGACTCTTGTTTGGCAAGAAGGTCCTCT
GCCAAATCATCCAATAATTAAGTTTTATGTTATTTGTATGAACGACCAAGCATGCAGTATTCCTTTTCAAATCAAAGGGAGGCTTAAAATTACT
TTTTGAACACTTTCAGACCATGGTCAGGTCACAGTTTGGGGGCTGAATTAAAATGATCTCATTCATGATCTAATTGAGGTTGGCAAAACCTTAG
CAGTACCAGGAGTACATAAATATAAATCAAAGACCAAAATTTACCAAATCATATCATTTGCTAGAAATCTGCTCATGAATTATTCTCCGATTGC
CAACCCAGAAGTTAAACTTTGTCTCCCCTCAGCAACAGTAACTGCGCATATTTTTTCAGGCTTCTGACAGGAAAGTTAGAGGATCTGAAAAGAG
AAAGGAGAAGGAAGATGATCACTGTGTCATCAGATATGGTACATTTGTGACCCAAGAAACTATGAGAGCTGATAAAGCCATGTACGTTTACAGT
GGCTGCTGCAGCCTTGGTTAGAAATTAATATGGGTTTCTAAAAACCAGACTTTGGAAGAGCAGGCAGCACAGACACATTAAGAGTGTAAGGGGA
GCAAACGTTTCAAGAAGGGTCTGATTGTACTTTGCTTTCATTTTTTCAATTGAGGAATGTAATCATCATAATGATTCATGATTATTTACAAATT
TAGTTCTATCACTGAGCTGGGTCCAGGGATTGAGATTTGAAGTAGTGGTTAGAACTAGAACACGATACGGAGCTGTTAGTGTGCATATTCAGGT
TACCTTTCATTCTTTCTTTCTTTTTAACGCTGCTATAAAATTAAGACTCCAAATAACTTTTCCTATAATCTGTGGGATTAAATGTCATACTCTA
TTGTATCTAATCCATTTTTAAGTTGTAAATTTTTCTTGTTTGCTGTGTATGATTTCATCTTAAACCCACACAACACTTTACTATAAACTAGAAG
AGGTCTAATTCTACAGTTGAATCTTTTGAAGTTAAAATTTTAAATCCTGTGTAAAAGAAGATGTTCTTTTAATCAAACAGTAAACACAGAACTG
GTCAGTTGGCCACCTTTATATATCACAAATATATGAAGGTATACTTTTAAATCTTCAGGATAAACTAAGGTGGGAACATTTAAAAATAATCTCA
GGTGAGAGAGGTAGATGACCACATTCCTGGGAATAGGTAACTGTCATTAATGATTTCCTAAAATGTATTCTACATTAAAAAAACTGTAATGGAT
TTCTCTTTAATAGTTACGTTGCCCATAGACAACATAGTGATTTTTGTTCTTTATATACTGTTTCTTATTTTTTTATAATAACTCAAGGAAAGGT
ACTCCTGACAAGATGATAGCCACCCCCTCCTCCCAAATATGAAATAATCAGTAATAGATGACAGATTAAAAATATTGTGCTATATAAAATCTGA
GGTTTTTTTCTTTCTCTCAAATGGATGAGTAAATTTCAAGGAATTCAGTGCCCAAGTGATCTATAAATGTAATATATTGCTTTCAATGTTTCTT
AGTTTAAGAAAGTATTTATTGTCTTGTATATCCAAGTAACTCTAATCAAGGAATTGCAATTGCCTATTCTTGGAAGTACCTAAAGTTTCTATTT
TAGACACCATGCACATCAATGGAAGTGTTTCACATCATGAGAATTCTCAGTTTTAAGAGGTGAGGTTTATATATAAGGAACTCAGTAAGCTGTG
TTTTCATAATAAATAGACCCATTAAGAGGGAAATATCTCTGAGCAAAATGGTTGTCCTCTAAAGATTTCCCATCACCTTTGCTCTGCTGATGGG
AATTTGAAAACTAAATACAGCCCATGTGCACACATTCCCACATGCACACAAACACAAATGCACCCTAGGATCCAAACTCTGAGAATTTTCCTCT
TTAACTTTCACTACAGTCATCTAGCTTGTGAATGAAATACACTTAAACAAAAATAGTAGAGGCGAAGAATTAATGTAGATTCTTGTTTAGTAGG
ATTTATTGTACGGGGGAAATGTGTTCCCTATGCCACATGATTCTTACGAAACTTACACCTATTAATCTAAGCTTTAAGTGTATTATTTAGCATA
AATTACTAGCTTTCTGCTTTTGTACTGTTTTCTCCCATTTTCCTTCAGTATTACCAATCTTAACACAGATTATCCTAAACTGATTGCAAATTAT
TATGGTTTTTAAAACAGCATTCTGGAGTCCTGAATAAAGATGGTATTTTAAACTAGTGTTTACAATATACAGAATAAAAAAGGGTGGGGGAGAT
TTAAAACCATACCGAACACTTAGAAATGTGTGGAGTTTTATACTTGTGCAGATAAGTGTGAGGTAGCCTTGCCCTGTGAAACCAAGACCACAAA
TGATACGTGTCAGTAGGTTCTTTGATAGGGCTTCTGGCCACAGTGTTTTTGTGCTGTCAAACTGCTATGTTGAACAAAAACCTCCTCTGCGACA
GAGTGATAATGCAGAGACAGGCTGGCTTCAACCAGTCTCAGCAACCTCCTCAACCACACCCCCCACTCCCAGCCCAACCTCCTACCTCCTACAA
TCCAGCCACCTCCCACTCCACTTTGCCCCTTTACCTGAAATTGACATTTATATAGAGGTGCCCAGGATTTTTTAAGGTGTCAGTGGTGAGTAAT
TGTTGATACTCCCCAAATCTTATCAATCAGTACTCTGAAACTGCAGGCAAAGTCCTTTTTCTCCCACCCTTTATCTAGTCTTTTTAAATGGAAC
TGGACTGAGCTTCTAAACTTTTGCCAACTTGAACTTTTTCCACCTTTGCTTAATCACTTGAAAACCACTTGCTTTCCTTGTCTTTCCCCAAGTC
GCTTTTTATTTTCTAATGTTCAAGCTATTACATTGTGCAACACAGACAAAATTTTGCCTTAAAAAATCCAAACACTATTACCTCTTACTTTTCA
CTTTCTAGCATTCTTTTAGGGCTAACATTTCAAATGTATAGTCAACTCGTCTTTTTTTTTTTTTCTTCTCTCTCTTGGAGCCATTATTCTCTGA
CTAAAACCTCTTTACTGGTGCCTTTGTGTGTGCGTTTTTTCCCCCTGACATTAAATGAATTTCCATCTTCTACTTCTGCAATCTCTAAGAAATA
GATAAACCATTATATTTTTTCCCTGCTCGTATTTTAAAGTGGTGATCCTGACAGCAGGACGATTATTGCACTTTATGTTTTAGTGGATGTTTGC
TGTTGCTACTTACACCAACACTTATCATAAGACAGCAAGAAAAGACTACTCCCTGGTAATTAAAGTAATGAAATATTCATTTACTCTACCTTTT
CTGTAGTCTCACAAATTAGGCTGCTACATTTAATGCCTGCACTGCCTCTGTTTTTATTATAATGGCAGCTTTCGCATCTGCAATTAGGACTAAT
TCTGACAGTTACAATTTCTGAGGGATGGTAAACAGTGAACGACATCTGCAGCAGAGGTATTACACGTGAAAAGCCCTTGTCTCTTTAACCTTGT
TGTTTTTTTTCTCGCAGACGTTACCTTTTCCCGATGGTGCAAAATCGACTTCACATAACTCTTCATCAGTTTCAGGGTTTTCCCTGGAGGTATT
TGCATCAAATCAGCCCAGTCAAGCTTCAAGTTGAAATTGGCAGGCCAGAAACTCACAGGGTAGAGACTGGAAAAGAGACAGCCAAAGTACAGGC
AGCAAACTGACAGCAGCAGAGTGAAAGACAGAGGGGTGGGGGGAGGTTGAGAGCGAGAGCTTTTCAAGTTTAGTGTGCGGGAAATCTCAGCTCC
TCCTGTACTGATCTCAGCAGCACCATATCCTGAAGGAGCTTGAAAGGTTGAGATTCACAACACCTTCAGCTAATTCTTACCACTAGGGCTCACC
AGCCTTGCTCCGGCGGTTTGCTCTCACTTAATCTGATTGGATGCCTAAAGAGGAGTGGGCACACTTAATTAAACGGAAGGAAAGTTCTGAGGCA
CCTCCCCAGCACTTATTAAAGCTTGAAAGCCAAATCTCATGATCAGTGTTATAGGAGGAAATTAAGGTGGTTTGTTTTTTTTTTTTTTTCTAAT
TGTTAACTTGTAGGAAAGTGAACAATCCCTCTCTTTACCAGGTTTGTTTTCTTGGGGGTTCTCTTCTGAGTAGATTAAATGTTCACAGAGACTA
GAGCTCACGGGACTGTCCATTCAAAAGCTAATGCATTTTGGCTAACAAAAAGCCAGTACAAGTAGCAAAAATTATATTTGAAGAGATACTCCTT
TGAATGACATGGGTTTGCAAGTTCTCTGTCAAATAATGCCCTACTATCCTGGTAATATGAGAACACGTATTAAGTTGTTTAAAAGGTCAAAAAA
CATGGCAAAACCTTAAGATGTCTAAGATACTCAACTTTTACTCTCAACAAGGTCACTTTAAGAAAAATTCTATGATTGCAAAACTTGTATAATT
GCTTTTACAAAGAAAACAAATATGAAAAGTGTTTCCTTTGCCTGAGAACATTTTTTCTCCTTGATGTCTTAAAAAAAAAAATTGTCATCCTTCC
CCGTCTCTTCTAACTTTGGAGTGGGAGGGGGCTGGTGTTTTTACTAGATGATTTAAAATCCTCCTTACTTTCCAACTAGACTTGGGGGTCGAAT
AAGCCCTTGCTTTCAAACCGTTTTCTAGCTCTGTGTATGACAGGCATTGGCTGATCTGCTCATGCCCATTCGACAGGCACTGTTGAACTAAAGT
GGTGTGACGGCACAGTTTTGTTAAGCTGTGAACAAAGGGCTGAACATCCCTTGCTGGCATCATCCATGTGCATATTGGCCTTGTCACAGTTTTG
CTTGATTTAATGCTCTGTTTATTCCTAGATATTAAAACTTAAATTCTGAACAATATGTTTTCAAATACTACATACCAACTTTATTTTAAAAGCT
TTAACTTTGAGGACCCTAGGGTCCATTTGCTCATCCAGTTTCTGTGTAGCCAACATTAAAACACAGCTTTGGAAATAAATAGATTGCCAGTAGC
CTCTTACCTGTTATCCATTTAAAAATGGGATTCCAGTCTTTAAAAGTCTTATTAGGATAAAACATTTATTTTTCTTTGATCATTGGTTGAAAGT
CATTATTTATTTCCAACACTGGATTGTCACATAAATTCCTAAGGAAGAGTTACCTTGTGTGTACTCAGTGCAGTCAGAGTAGACTTTTTAAAAT
GACAAATTATTAAAGAGATATTCAGTAATAAAAGAAAGCCTATAAGAATACCTATAAGGGTACGCACATCACCACTGACACAAAAAAAAAAATC
AAGGGAGTTTATGTTAAAGTGAGCCCTATTTAAGAGATAGCAGAAGAATTAAGATTGAGACTTAAAAACAAAATAATTGTTATGAAAATCCCTG
TTCCAGAGGCAACAGCTCGCCTATCTAAAGAAATCTTAAGTTTGGTACTAAATTTAGATTCCAGCGTCCCGTTGAATGTGTCTTGAGCTTTATC
TACGCTGGGTTTTTGTTCCCCCTCTCCCTCTCTTTCCACTCTTCAGGTTTTCTTCAAGTGTTTGCTAAGAGATTAGTTAGCAAGTTTATATTGA
GGAGGTAGCCATGGGAGGCAGCTCTCTGCTTGAAGGAACGCGCAGTGGGTCAGAGTGAGAAGATACAGGGAGCTAAGACTGACTAAGGCCTGGG
ATCAGGTACTTTATTTTCTCTCAAAGACAAACCGCCCATAGAAAGGTTGGTGGGATGGCATCTGCCAGCAGGAGCACAGATGTTACCACTTTTA
GAAACTTCCAAACAAGCATTATTGCTGGAATAACTTTATGAAAAAAAAAATCTGCTGCCAGAATACTCCTTTTTCATTATGTAAAGAGGCTATA
ATTTGAGATATTCAAGGTTATTTTTACCTCCATTCATAAAACAGCATCTTTCTTGAGAGCTGTTACGAGTTAGGTGAGGAAGGTGGCAAAGGAG
AGAAAAATACTCAGCAGCCTAAAAGTATTGCTGCTGCTAATAACCGGTGAGACTGCACTAAGAATCTGAAATGGAATCTCCGGGGTAAAACACA
TCCACATTTCTACCTGCCCTAAGCGTGCTTTCTTAGCAGTGACTGAGATGAATTGTCCATTTCTTTTCAGTGGTAGTTTTTCTCCATGTTCACT
TATGCCAAATAATTCCCACAGCCAGAAAGAGTGATACAGGTCGGGGTGGCTAGGAAAGAATAAGCCTTTATTGCTAGTTTTTGTATAACATCAG
CCATGAGAAAGACACTTGCTAACAGCATAGATTGCAACACACTCTGTCGCCTCTTCTCTTAGTTTCATGAGAAAATTTTGTCCAATCCCATAGG
TGTGAACAAGATTTAGACATGTAATCTCCAACCTGGAAAAGAAGAGAGCTTTTAATATATTAATACGGCACATTCTTACTTAATATATAAAGTG
AATTTTGGCATCAGTTTGCCGTTTCCTATTAATACTTTGTTAGCAACTGTTTATGGAAGAGACAAACAGCAGTTCATCCAAATAGTCCTCCTTC
CCCAACCCCCAACCCCTATCTTGGTATTAACTGCTTTCTCCTGTCAGCTTATAACATTCTATCTTTAAATTACTCTAAATGAAGTCATAATTCT
AGTTATGTTTTGTAACAAGTCCATCGCTAGGACCAACATGACTTCTCACCTTTTAATGAACTGTTCTACTTTAGGATCTTCTAAGGAATAGAGT
GTAAAAAGAAGCAAAGCAGGAGATGAAGAGGTCTCGTAAATAGTTTCCTTTGAACATCCACAATTGCTTACATGGCCTTTTCCCCTTTTTTCCA
GGCTGCCAGAAATGGCCTAGCCCAGGACTCATTCAAGTGAGCACATTTCAGAATGACTCATTGGATGATTGGAGGGAGACACAAGCCCTACGAT
ACCAATGCAGTGTATTAATGGAGAAAGTTCCTGAGATTGGGAGTGGGTGTGACATTAGGCTAGGGTATTCCTGAAGGGCTCTAATTCATGTGAG
AATATTAAGTGTAGATCTTCTAGCTATCAGTTAGCGTAAGGTTTCATAAACAGGTAATGATGGCTCTTTCCCCCCGCTACCCCCCCATCCAATT
TCTAGCTGAGTGAGGAACAGGCTCTGCTTCATATAAATACGATCTTTGAATTTATCTGCAATGAGGTACAGCTTTAATGATCTGTATCTGCAAG
CCAGGAGCATGAATGTGATCTTATTGTATCATAGTGTAGAGATGAGATAGGCTAGCGCTACAGCACACCAGTGCATTGTGCCTGGGTGTCGGGA
GCAATCCTTCCATTTCCAAAGCAGCAACTCTTTATTTTTCATCATTGTTTCTTTACCTCACTTGTATGCTCCTTGTGATACAGATCTGAGTGAG
TTGTGCAAGCTTCAGACTTGCCAGCCCCCAGCTCCCAGGCCTAGTACAGAGTAATTCATAAACTTACCATATTAGACTATCAGATTGAAACCAA
GGGTTTCTACTGTGGTGGAATTGGAAAGCCCATTGTTTTGATTATCATTTCATCAGCCCCAACTGATACTAGTATGCTCAAGGAAAACATACTT
CAGTTACAGTTGATTTTCAGATTCCAAAATGGTGCTGCAAAGCCTGTGCATCTCTAATTATATTTTATGCTAGGCTGTGCAATACCAAAATATA
CGCCGCAGCAATCAGTGAGTGAACAGTATCAATCAATTTGTAAACAGATGAAGTCACTCATTCCCAAACTGGTTTTCCAAAAACAATTACTAAA
TTACAATAGAAAAAAATTGTACAGTTACTGTATTTGTGTGCTGTGTTACAATCAACATACCAGGGAAACAAAACAATTTAATGCAGAAAAATCA
TCATTCATCTACATTATAATTGCTTCTTTTTCACATCTACAGGGCGGTTAATTAAAATTGTGATTAAATGCGGTCGCGCTGCCTAAGATGTCTT
GCCCAAAACAGGTGGTACAGAATTTAAAAGTAAAAAAAATGTAATTAGCATTCGAAATTGAGAAATTGCCTGTAAGAATCAGTGTTAATTTCAG
TCAGTGATGAGGTAATTTATAAATGAATGCAGTGGAGGCTGTGCAAATGCACAATTGCCTTCCTTGCCATTCTTAGCATTCTCAGCATGCAGGT
GAAAATGTTTTCAAAATATTGAGGTAATTTGAAAATCAATCCATGCATTCTGTATCTTTTTTCTCCCATTTGTACCAAATGCTGAACAAATGCT
GCCATAGATGCCACAATCCATCAGTTTTTTACTTTCTAAAAGTAATGCTGCACATATTTTAGACCTAGATTAAGAAATATAACAACACAAATTG
CAAGCCTAAAATTGTTACACGGGTCAAAATTGTACACCTGAACTTCTGACTAATGGAGATTTGGTTGGCTGTGAAATGCTCCCATCAATAAAAT
AGAGACATGGGGGGAAATCTGTCCATGGTATAGGTGGTACAAACAGAATGACTATTAACCAATCCAATAAATACTTGAGATTAAATTTTTAAAG
TTTAAGAAAGAAACAGAAAATTTATAATAACGCTGATATTTTTCTTAGAATCTTGAACCGTAACTTTGTTATTCCATCTAGAAACATCTAAAAC
CAGTTAATAAAGTTATCACTAGAAAAAGCGTTTACAGAAATTAACTGTTTTTCAATGTCATCTTTCCTTTCAACAAAGATGCAACTTTCTACAA
AACATTATGCTATCAACTTACTATATTTCAAGCGTTCTATTGGCAAATGAGTGGATTCTATAGATTAGATACTGGTTAAAAATTATTTCTATTT
ACGCTGTATTTTTACTATTAAACCGCTAGACTTTTAAAATCTTTTAATGGAAGAAATTTAACTTTCATGTGCTTATTCTGACCCTCTGAACAAT
ATGAATTTAAAATATTTTAATAATGAATTGAGAATATGGGGTTTGAGTATCTGGCTTGATACCACTGGAGTTAAGAGACTTTGTGGGGCTTTTT
AAGCATCCTCTTCTGTCTTTGGCATTCTGCAAAATGATCTCTCCATATAAAGCTCATCCTAAGAATATCCTTATTTAATTGTGGCAGATCAATC
TGATGATCAAATTCTCTCATGTAAAATATTCTACGTGTTTTTTGCTGTTTCAGGCAGAAATCAATAATTTCACTTCACTCTGAAGTTATGTTTT
GTGGTAATACCGACATTTTCAGGAGAAAGGTGAAAAATAAGAAATACTGTATCTCTCCGAGTGTCATCTTTATTCATTCATTCATTCATTTTTA
GAGACAGGGTCTCACTCTGTGGTTCAGACTAGAGTGCAGTGGCACCATCCTAGCTCACAGCAGCTGAGATTCCTGGGCTCAAGGGATCTCTGGG
AGTATCTTTCTATCCTCTGATTTCAGGCCTAGTTTCGGGCCTTCCCTCATCACCCTTCCTGATCATTTTTAGGACACAAATGAAAAGTGCAATT
TAGAGGTGACAGCTCAGCTGGTTGTTCCAAGTGGCATGCCATCGTTTCTCTCATATTTAAATCTGTTTCCCATTTAATATTACAAGTAGAAATA
AAATGAGACCTCTAAATGGAGGAAACTGAAAGGTCACCAGTTAACTCCATGATCCCTCTCCTTTCACCAGCTCTTGCCTCCCACTAACTGTGAT
GATGACGATTTCATATTCCAGCAGTCAGTTCACTTTCACCGAGGGTGGGTGCAGACTTCATCAAAGAATGGCTTCCTGGATTTCCTTCAGGACC
TGTGAACATAAGATCCGCAACCACATGGGGCTTGGCTCAAATTTCAGCAGAGTGGTGGGATGGAGGAGGTATTATGACGTGCTCCAGGCTGCAC
TGGTTCTGAGTTTACAAGGGGTCACAAGGGTTAGAGAATCACAGATGTCAATAGAGCGCGAGGACCTTCCTACCCCTTGAAATATTTGCAGTAG
AAATCTTACCTCTTTACGTTTGGTTCGTAGTGCCCCATGAGCTTAGCACGGCAATGTGGAGTTACTTGTGAGATGCCACCAATGTTTCATATAT
ATATATAGTATATTATATAATATATAATATTAATATATATTATATAATATATAATATTAATATATATTATATAATATATAATATTAATATATAT
TATATAATATATAATATTAATATATATTATATAATAAATATTATTAATATATATTATATAATATATAATATTAATATATATTATATAATATATA
ATATTAATATATATTATATAATATATAATATTAATATATATTATATAATATATAATATAATATGTATGATAATGACATATATATATATATGTTG
AGAATGACAGTGTCTTTTTATACAAGCTCCCTCCACCAAGTATCATCTCTGTACTCTTACACGGTCATCAAGATTGTCAGAAAAGGCCTGGACT
ACATGCCTGTCCCAGCACTAGATTGGTATTTTTTAAGTCCTTTTTTGTTTCAAAGTTTGCCATGTGAGCTTAACTCAGTTCATTCTGCTGTTAT
TACAAGCATCACCTCTTTGAAGCTGTTATCAGATTCATGAAAGAAAAATGCGCATTTTCTTTTTCTACTGAGGGCTTCTTTCACCTTTATTCCT
GAGAGTGATCAGAGCTTTGAATTGTAAAAAAAGAAAAATTTTCTCATTGTACAATAATTGAAAAGTATGTCATCAGGTCCTTTCTAGGTCTTCC
TGTGGTATGTCAGCAGAAAACTACAGTGTTTGTTCATTTTGCATTAATTACACACCTACTATGTGCAGGCTCTCGCTGAGGTCTGTGGATCAAA
AAATGGATGCTAGAGCCTGGGCCCCTGAGGAGCTTGCTGTCTAGTGGGAAATGGGGGTGCATTTAAGATATGATGAAAATGTGGCAAGTGCCAC
AAAGAACTTTCTACCCAATACAGAGAGAGTACAGAGGATGGCACAACTAGATCTGGGAGAAGGTGGTGAGGGGTCACTTCATGGCAGAGGTGAC
TTTTGAGGAGACTGTTGTCATATAACATGGATTTCACCAGCAAGTGAGGGCACAGCACGCTGGGCTGAGAGGATGGCATGTACAAGCCAGGACA
AAGGGTGAACAGCGCAGGCATTGTCAGCCAACTGAGATGTCTGAAACACAGGGCATGAGCAAAGAGGATGGGCTTGATTTGAGTCTGCAAAGGT
GGTCAGGGAGCACAAGAATGTCCTATCCCATATTTTTGGCTTGTATCCAGTAAATAGGTGATGGTGAGCTTGCCAACCATGTTTTATAGAAGAG
TGACATAATTATATTTGCGTTTCAGTGACACGTCTCTGGCAACTGTACGAAAGATATTATCATCTAACATTTGTTGAGTACTTAACAATGTGCT
GGGACCCGTGCTAAGAATTTTGCCTATGTCATCTGATCTCGCCCCAACAATAATGCTGTGAAATGGGTACTATTATTTTCATACTATTTTTACT
GATGACCAAATTAAGCTTTAGAAAGCCCATGAGCCTGGAGTCAGGAAGAAAGTCTACGGTTACATCTTACTGTAATCATGGAATTTCTACAATT
ACAAGCAACACACGCAATAATGATCATTGACTAGAAGGACATTTGGAAAAGAAAACCCAGCGTTTCAAAAAGAGTGAGGCAGTTTGATTCTGTT
TCTCTGAGTGAATAGGGAGACTAAGTCGCGTGTAACGATGTTTCATCCTCAAAATTTATTTTTTCTTTCATTACTATGTCAGTTTGAAAAAGAA
AATAAAAAATGGTTGTTGATCCTGAAGAAACTAATCCTAGAATGTGCTACATCTTTGACAGTTTGGAAAGAGACAGTTAACATACAACCCTTCA
AAACAGTGGAATTTGGTAAACCCTGGAACACTGTTCTTGCTGATCTAATTCATGACATTAAGGCCTTCCTCGGCCTTATGACGAAGCAGATGAA
AATAATCCTTTTGCGATGACTATTTTCCCCCTTTTCTGATTTAACTCTTCCATTGCCTAAGACTCACCAATTCTCAAACTTCTAAATTCTCCAC
ACTGCCTTATCTCTGCCATCCCATGGCAAGTCCTAAAGCTGCTAACATTTTGTAACAACTATGTGACAAATTGTTGTGCTTTTATTCAGGTCGG
ACATCTTTGCCAGGCTTGCTTGTTGTGTTTTCTGTTTGGCTGTGTCATGCGACCAAAGGTGCCTGCCTTTGTACAGCATGTCAAATCTGGTTAA
CATGCAGCCATTTATCTTTAAAACCACTCCTGCACAATGGCAGAGGTGGGCCAAGCCACAGGGCTCTAAGCACAACCCAATTTTTCAGACCATC
TTTTCTGCATATAGGGAGCACGTTGCACAGCAGACAAGACATTTTGTGATTTGGGGAATCTCTTACAGACAAATTGTAGAGTGCAAGTGCAGTG
TAACTGGACTGCTGGCTGCCCACTGAAGTGAGGAAAATCAGATACAGCACAGGCTGAAAGAATGAACCAAGAATTCATATCTCTGTGCAGAACA
TGCACAGTACTTTGCTCACACAGAGAGTTCACACATAGACAAAAGCATGTTTGCTTGTGGGTTATTTTCAGGGGATTCTATAGCCACAGTCTAG
GCGTATATATTTGCACGTAAGCTTTCTTTCTGCATTTTGTACTGGTAGGTGTGTCTACATGTGAACATGTAAACATGCTTATATGAGAAGACAA
TTTGCTGTTGCTGATTTAAGACACGGGAAAGGCCTCCTAGTTCACTCAGGCGTTTCCCTCCTAATGTAGTAGAACCACAATCTTTTTGTTTTCT
ACAATTGTTTTAATAAAGGACTCTGTGTGTGTTTAAGGAAGACTCAGAGCCAGAGTGTCAAGTGCTGCAGTGAAAAATACTGAAGATTTTTCTT
TCCATAAGACAGCTGCAAACTTTATAACCCAGTTCACTCTGCGTTTTCATTTCTCAATTGTTTCAAGGTCTCTTCGGAATGATTTGCCCTTAAA
TAACTAGTTATCAACAAAGGAGAAAAAATAGCAATAAAAAATGTGATAAACCTGCATAGACTAGTTTAACTAAAATGAAGAATTTCCTTCCTTG
GAGCAGTTCAAGGACCGAACATAGCAAAACTGTGTGATTTAAATGAAATATTCAGAGTGGTCAACATAAATACACATCTAGACAAGAGATAGTA
TATGTGTGTGTGTATACACTGAGGTTACTTCTTGAAGAAATAGGCATTTTTTCCTTTCTTTTTGCTTTTTCTTTCTTTTCTTTTTCTGAGTAAA
GGACCCCAGAGATTGTCTTTCACTTTGAAAACAATCTCTAATATCAGGAAAATGCGCTGTTTGTTTATGCGTACATAATGCAAGTTTGTCCGCC
TGGGAACTGCAGCAAATGGGAGAAGTATGCATGGATTTCACAGGTAACAGGATTACGTGGGCACATAATTGATCCATTTGGAGAAAAAAAATGA
CAGTGCTCTAAAGATGCCTAGGCTTTGGAGATGGAGTTTATGAAACAGGCATTTGTAAATTCAAAGGAATCAATATGTCAATTTTATTACATCT
CAGAACCAGCTGGGCATTAAAGGAACAGTAGCCTTAAAAAAAATTACAGCTGCATATTCCTACGTTGTCTACCTTTCTTAAAGGCCCTAGAATT
TAAGGAAATGAACTTAGAGATGTGTACCTTTTCAGAAATGAATTTACATTGTCCAAAGAGAACAACAATTTAACATTATGTCAGTTATCAACCT
AAAAATTTGATTGAAATGTCTGCCTTATCATTACAATTGAAGTCTATTAATAGATACAGATAATAAGCAGAATAGTGATTTTCACATAGGTTGC
ACTGTCAAGGATTCATTGGAATTCATATTTGCATTGTGCTTTTCAACAGTCTAGATTTTACCTCTTCACTGATGATAGGACATAGTTTAAGAGC
TAGCCTTTGGGTGTCCCACAGAATTGGGTCTGTGCATGTGTGAAATATTCCTGTTTTATTAGAGTTTTTGACAATCTATTTTTTTTTTTTTTTT
TTTTTTTTTTTGACACGGAGTCTCGCTCTGTCGCCCAGGCTGGAGGGGAGTGGCGCGATCTCGGCTCACTGCAAGCTCCGCCTCCCGGGTTCAG
GCCATTCTCCTGCCTCAGCCTCCCGAGTAGCTGGGACTACAGGTGCCTGCAACCACGCCCGGCTAATTTTTTGTATTTTTAGTAGAGACAGGGT
TTCACTGTGTTAGCCAGGACGGTCTCGATCTCCTGACCTCGTGATCCGCCCGCCCCGGCCTCCCAAAGTGCTGGGATTACAGGCGTCAGCCACT
GCGCCTGGCCGTTTTTGACAATCTAAAAAGACCAAGCTATTTTAAATTATTAAGTTCCTAAATAATAGGATTTGGACAGCAGCCATAAGAGAGG
TGAATGGGATCGCAATTTAACATACTATTTTAATATCACAAAGAGGTTACACAACTAAGCATCAAAGTTAGAAGCTATGAATTGGTCCACATAC
TCTGCATGTGCTGTCTCCTAGAGGCTGCAAGGCGAGCTGCTTCACTGTTTCATGTTCCAATCTCCACCACCCAGACTCCTGCTCCAATAACATC
TCTAGATCTATTCACTTGTAACAAATGGAGCCCAGTTCTCCTCCCTCCCCAAGGAGGCCAGACACTCGATACAATGAAAAGAACACAGGATTCA
GACCCAAACAAATTTGTGTCCACATTCTGATTCTGCCATTTACTCTCCATGCCATGTGCCCCTCGGTGCGTTTCTTAACCACTCTGAACTTCGG
TTCTTAATGCTTAAAAATGGGGAGCAGTATTTGTTTTATAGAATCTTTGGGAGTATTACACAAAATTCTAAAGACAGTGTCTGGAGCCTAGAAA
ACGCTCACTAAATTCTCATTATGACTGTTTCAAGGTGTGACTAATCAGGATGCCAGGAACCCTACGATGCAGGCACTTCAGTTTCTGGGCCTTG
CTGAAACATTCTTGAAAAGAGGAAACAATAACCGTAGCCTGAGATATTCGTTCCTTTTTTTTTTACTCTTTGGAGAGTACTTAAAAAAAGAAGA
AAAAAGAAATAGTCATAGTGATTGGCATGCTATTCTACCTCTACATTTCCAGGAAATATACATGGTACTTTTTCTTATAGAAACAGCATCTCCT
CCAAAAAGAATACATTCTGACATCACTTTTGTCACAGCACTTAAAATATCTGATTCCTATTTGTCTCCCCACAAATGTGTAAATGGCTTGAGGG
CAGTCTTGCCCATCTCAGCTCAGGCCATGACATACAAAAATTGTTTCGTTAAAGCAAAGGTATAATCAATACAGCGGGTATATTAAAGATTCAG
TATACATTATCATATGTAGAAAAAAGCTTTGGCTAACTTTATGTCAGATTCCCTACCACCATATTAATAACTTTTTGAAATTCACTTTGTGACA
GCTATGTTATTTTGAATACTTGAGTGGGTTTGCTTTTTCTTTTTGTTTTTGTTTTTAGTTTGTATTTAAATCAACTGGCTCTCTGCCTTTCTCA
ATGACACCTTTGAGACTTGGGAGTCAGGGAGGCAGAGAGGCAGTGTGGGTGCTCGGAAGAGTGTAGGCTTTGGACATCTGGGCTCAAATTTGGT
TTCTACTTTCATACAAGCCATAAGACCTGTAGCAACTTGTTCAGTCTAACTGCATCTCTGCTTCTCATAATAATACTCATGTTATAGGGCACTT
GGGAAGAAATACTGGTAACAGGTAAAGTGCCAAGCACATAGTAGGTGATTAATGTTCTGTCCCCTTCCTCCACTCTTCTTTGTCACTTGACCAT
TCACTTTGGGTTGGCATTTAGTGAGCTCCTTCTGCCCGTCAACAAGTCAATCAACATTTAATACATACCATGGGTAGATATCTTACCCCTGTTC
CAAATTTCATTATGAAAATTGGGCTATATTCTACTCAAGGAAATGAGCATGCAGAAGTACCTTCTGTACTTAGAATCACAATCATTTTAGCTAT
TTTCCGCTTAATCTGACGGTTTCTTTGTTCCCATCATCTTTTTTAGTGAAGCACTTTGGTGTTTCTGCAATCCTTTGCTATCATTCTGTGCTTG
GGTATCTCAGATCTCTCTTCTCCCCCGATCCTTTGAAGACATCAGAAAAGAAGAAGCACAAGGTCTCTTGACTATACGTTTGCCACACAACTGT
GTGGATTGGTTACTTAATAGAGTTGATTAAAACACTTTGTTTGATTCTGCTTAAGCATACCAGAAATACATTCAAAATCTACAGCTACAAAATG
TAAAGAAGGTAGTCCAAATATAGTACTACATAATGAGGCTCCCTGTTGGTAGTATATAAAGCAGTTTAGAGTATAGCTTCAAATTCTAATGAAG
ATCATTTTAAATCTTGCACTGAATTGTGAATTTAAAATTCCCATGTTCTCCATAAGTATCTGAAGAGGGCCATGTAAATACCATAAGTGAATTA
ATTACTGCCTTAAGTTGGTAAAGCATGTGTGAATCCAAACTTTATTCTTGAACTTGCACTTCTCTTCCCAAGGTTTTTTGAACCATTTCCTATA
TGCTGAGTTCTGAGTACCTATTTAACTACTGAGGACATATGAAGAAGGTAGTTTTCTTTGCATGGATTAAATGTTTCAATCTCTGAAAATCACC
ACCACCACAACAAAAAGTCTGCCCGGCACCATCTACATCTACATCATCAAATTCCTAAAGCACAAAAGATATATTTATAATCTGAAACTTATAA
AAACATGACAAACTGTTACCTTCACTTTTGTGCCCCCTTTTTTCATTAACAGTATTAGGTCGTTTTTTTTTTTTTTTTTCTCTTGAGAAGAAAC
GAAAGACGAAGTGGCAAGAATATCTTTCACTTTTGCTCACGTTTTGGTCGTCTAATATTGGGACCACTGAAGTAATTGGACAACTTTTCCGAGC
TGACTCTTCTGAAGTAAACCCAAGCCAAAGGGAAAATGGAGCTGCTGCTAGGCTCAACTGATGGCGCCCACATGGCGCCCACCCCAGCTCCTTC
AGAGACTCGATCATTTCCACAGAAGGGCCAGAGGCCCTGCCAATATCCGCACATCACTGGCATGATAGACCATAACAGCTTCTTCTGAGCTGTT
CCGGTCTATCGTGTCAATGCGTACTCGGGGAGTCCTCAACGCACGGATCACTGACACGGCACTACATAACGGCTCCTTAGCAGCCATTATTGGG
TATCAAGTCAATGCCTACTCAGGGAGGCTTTATCCCAGAGGCCTGCACGAGCAGACACATTCCGAATACTAAAATTCCTTAGCAAAATTAGTCA
TTGCCCATGTGTGATAGCTGTTTTCCATTTTGAATTTTAACCATTTTGGGAAAAGGAAGAGAATGGGAAATACCAGTATAGTCCGAAAAGCCCT
TAAAAAATCAGTGCGAAAAGTGGAAGGGTGGAGTGGACATAGGAAGATGATGAACTTGTCATTTTTTCCTTTCCATAGCTTCATTTTATTGTGT
CAAAGTTGCAGAAAGCACTTCACTTTCATCGAGGCTCTTTTTAGGCAGGGTACAGCCAAGAAAACTTTTTTATGGCTCAGATGTAAATCCTAGA
AAACGTAGTAGAAATGATGGCAGGATCTCTAACTGTAAAATAACAGTACTGTTAGTAGTAAGTGTCATTATAATAACGTTATAGAACTCTGCAC
ATCAAAAATACCACATTTTATCATGATCTCTGCAAGAGATTTCTGTTGGTAGTCATTCTAGACTTCTAATAAATAATACGTTGAGTAGGAAAGG
TTCTGTGAGGATCCAAATAATAATGAAAATGACAGTGGTGATATTAATAATAATAGTACAGCTCCCATTTATTGTATCATTATTATGTGGCAGG
CATTTGTATACATTATCTCATTTAATTTTCTCAGCAGATGTGACAAATCAGGAAACTGTGGCTTAGCAAAGTTAAATAACTTCACTGATGTCAT
GCAACCACTAAGTGACAGTGGTGGGATTTGAACTATATCTCGGGAGAGTCATGCTCTTACCATTGCATTGCCTCTAATGATCATCACTTTCAGG
GAACTAGGTGTTTAATTAGAATATCCCAACCCATTGTCTGCTTACTGATCAGGAAACTGAGGCAGCGTAATTTTAGTAATTCCCCCAAATAATA
AAGAGAGTCAGAATGAATACCAGAAAGCAACAAGAGGCAAAAACAGCCTTTCTCTCTGACAACATGTTAACCACAAACAAGACATGGGAATTTC
AGAGCCTCCGTTTGTGTTTCAGAAGGAATGTCCACTGTTGCCATCAGTCCTGAAAAGATTTCCTTGCACCAAATCAAAATGGTTCGAGTTTGCT
GTTGTTCATATTTTTTTTCCTTTTGTGTTCTGCTTACTGACTGACATCTGCTATCATGCTAAGCAGGTGTTCCTATGTAAACGAACGTGCCCTG
TTTGTGTTTCGCCATCCTCGCTGCTGCGCACCCAAATCAGCAGCGTGATTAAGAATCCTGTAAATTTTTGAAAGGAAACGGATGGCTACCCTAC
TAGAACATAATTACACCGTTCCAACATCCGCAGCTCACTGCTGCAGCAGAGTTGGAGAACAGCAGAAATTAATAATATATAATAGCATCATTCT
AGTAATTTATAAATGAAATAAAGTAGAGAGGACATTCTGCTGATATGCATTTTCTCCTTAATTCTAAAAGACAAAGTATAGATTAGAATTTTAG
TGGGGTGAAACCTGGCCAGCAGTCTCCCACTAGTTGCCTGATTCATGATAGGAGTGTTTTTGCTTAGGGAAAAGAGCTAATATGTTTTATTAGG
CCAGAGGAAAAAAAGAAAAAGAAACATGAATAAACAAGGCCCTCTAAAGGCAACACTAAATAAAGGGGGTCTCAAAAAATCTCTGTGGATTGTG
TGCCCACTGTATTTAAAAAGATATTATTACTTTAAGACTTCAACATGTGCTGCATTTTTTCTTTCATTGTAATAACATACCACCATAGGTCACC
CCTTCTGAGAAAAGAGGGACTGATCAGTTAAAGATGTGGCCCAACATAAACTTTATGTTAAATAGCACTCGTGTAAATTCTAGAGATCTGCTGA
ACAACAATACACCCATAGTTAGCAATAATAGAGTAGATTTCATCTTGTATGTTTTATCACAATAAAAAAGAACATTCATGCCTTTTTAAAAACA
TTATTTTGAGTACCCAGTATGTGCAAAGCAGCCTCTTATTACAAATCATCGTTAGATTAAGGGAGTTGATGCTTTTAGTCAACCATGCTGGAGA
AACAACAACACTTTCTAAAGAGGCAAATAAACTATATGTTAAAAGTAACCAGTCCATTTCTCTGTCTCATAGGGTTGTGGCAGGCACTTTAGAA
ACTTTAATCTTTTTCTAACTTCATTTACCGTAGGTAACATGCCTAACAAATGGATCAGCTCACTTCTTTCAAGAGTTTGTCTCCCATGAGCCAA
TGCCCATCACACACACACACACACACACACACACACACACACACTCACACACCAGCAATTATCCTTAAGAAAAATAATATTTATAGCACTTAAA
GTGTTATTTAAGCAACTTTTCCTGCTTAATTCCAAAGAATGACAATACATCCAAAAAAAAAAGTCATGGATTTTCTTCCTTTAATTAAACTTAG
AGAACATGAAAATACTGTTTGCCTTTCAAGAGAAGCATGTCCATTTAAGATGCTTATTCATTTAAGCAGTATTCATTTAAAGCTCCCCTTGAGT
ATTACTCTATGTGTTTTGATTTCGTTCAGAGAAAATAAATTCATTTAATAGATTTATTTCATTAACAGGTATTTATTTAAGTGGGTTCCACTGT
ATTTAAATCTCCAAACAGTTTCCATTACTTCCTTAGCTCACTTGCTCCATGTTCACTAATTTTAATAAGCCTAAGGTGCCTAGGGTCTTATTGA
GTGTTCAGTTTGATCAATATGGTTAAATTAAAATTGTGCAAAATATAAGCAAATGTACTCTAACAACACAGTGAAAAATGTGCTGTGCACTAAG
CATTGATTGGTATGCTGTGGACACAGATATATGAAGCTGGGTGGGCCTCTGAACTACCTCGAAACTGCCACTCTCCTCCAAAGCTCCCCTGGCT
TTAGATACATCCATGTGATCTCCCACCCGTTCATGGACACAGCAGCTCAGATACCCCCATTGAATTAGAAGGGGAAGGACATTCATGTCAGGTG
TTTTGCTTTTTGATTTAATGGAAAACAGCATGTAAGGCTCACAGTGAAATCCTATATAAAGAAAAAAAGCTAAGATTTTATAAAAGTTCAACTT
TGAGTATTCCTCAAATGCTTTAGGTAATAAAGAATGCACGCCCTAGTTCTTTTTCACAGAATAACTTTTTACCGTAATTATCCTTAAATTTTAA
TCATTTGGTAGCTTGAATTCTTTTTCAGAGATCTTACTTGTCAAGTTTGTTTTAGCAAATTACAAAAAAACAAAAACAAAAGCCAACCTGATTT
TTAAAGTATACCAAAATCAGCTTTGCCATTGGTACTGCATTTGAAAACCTAGTAAGCTTTCTAACAAATTCATTGCTTTCTATATAATTGTAAA
GTTAAATAGATAACTCAATATTTTTAGTGGATTCCTTTTAGTATGAATAAGTTTGTATCTTCTATTCCTAAATCAATTTCTTTACCTTTCCCTT
TTTCTCTTCTTTTTTTCTCCTTTTTTGCCTGTTTTATGGCAAGCATACTTAATATTCAGTGTCAATCTTTTTAAGCTTTTCAAAAATCTACCAC
AATCACTTACCAATCATAAGTATATATCATTCAAAATTGTTCCTGATATGTGCTGGCTGTTGGGGCAGTTTCTTTTTCCTACCTGTTGTGTGCC
TTTGTACCTATCTTTAATCTCTTCTTGCTAAGTGATAGCTACCATCACAACACTGGCTTTCCTTGATAGCATATCTTCCTGCATGTCCAAAACT
TAAGTTCACATGAGCAGAAGTTGTTCTTATCAAAGGTAAATCAGAAGTTAAAAAAAAAAGCAGAGAGATAAAAACATCAAAAATGTACATCTTG
CAAGCAATGATGTTGAATTTCGCAGAAGTGTGTTCTATTGAAATGAAAATCATTTTCTAGTTTAAACTGATCACGCAAGTTTATATGCATAATA
AGGATTTGAAGTTTCCTATCAAATTAAGTACAGTGTTTGAGCTTCCTAACATTTAATAATACCATCACCAATACACCTGCAAAATAAAATAATG
TTTGAGCCCCAAAATCATCAGAAATATGTAGACCAATACCAGTTCAGAAGAATTGACTTTATTTTTGTAATGGGGCCAAGAAAAATTCTTAGGT
TTTTGTTTAGTTTTGTGCTTGTTAATCTTGATTTTTCCCAGTGTTTTGCATGTTTTGAGTTTTTAATATCAAAGGTACTGCTCTTTGTAAACAA
GTGTAAGTTTACCCAGGCAGCTTCACTTACACCCTTCCCTTGTAATCAGATGTGAAAATTACACCCTCCCTTTTGGATACATGATAATTACACA
TTTCAACATGCAAAGAATTGTCTCTGTTTCAGTTGTTGGAATGAAAAAGCTGAGACATTTCAGTAAAATATATTATTATATTAAAGATCATTTC
CATAATTGTTTTCGGGATTTGTAATCAAAACCATGATCAAGGTTGTCTTAGTTTTAAAAAGAATACACATGGAAATAAGGAGAGGATAAAATTA
ATTGCCGAGTTCCACTTGAATGACCATTCTTTTATTTTATGAAAATACAGTTAAGTACATTATATTCTTTCTTTAAAAAAAAAAAAACCTTCTA
GTACCTTATTTGACATTTTCTTTCTCCATGTTCGTTGAATTACCTATTTTTTCAGGTCGACCAAAAAAATGTAGAAATACACATTTTTGTGATC
AAGGTGAAAAATTTACTCAGAAGAGAGATATAAATACAATGAAGGAATCTTTTCAAGAAAAGATGTGTTTGTTTTTGTTTGCAAGAGTGCTTTT
CAGCCAAATATAAAATTTTGTAAACTCAGGTAAGGCACCTCACACTTCACAATCAGACTTCTGGATTCATCTAGAGTATCACTCATATCATAGG
TAAAAATATATGTCACTATTGGTATAAAATACACTCAAGCCTTAAAAATGCAGAGGTTTTGGCAAATAACACTAACTCAAAAATATCTTGTATA
TCCAAGGAGAGGGTTTAAAATCACCGATTCCTTTTTACTTTCTTAAGCTAAGAAGAAAAGGAAGAGAGAGGCCAGGCGCAGTGGCTCACACCTG
TAATCCCAGCACTTTGGGAGGCCGAGACAGGTGGATTGCCTGAGCTCAGGAGTTGGAGACTAGTCTGGCCAACATGCTGAAACCCCGTATCTAC
TAAAAATACAAAAATTAGCCGGGCGTGGTGGCGAGTGCCTGTAATCCTAGCTACTTGGGAGGCTGAGGCAGGAGAACAGCTTGAACCCAGAGGC
GAAGGTTGCAGTCAGCCGAGATCATGCCGTTGCACCCCAGCTCAGGTGACAGCGTGAGACTCTGATCCAAAAATAAATAAAGAAATAAAAGAAA
AGGAAGAGAGAAGGCAGCATTAGATGGCTGATGTTATGGCTTCGTGAAACAGTTTATGGCATAAAGAAATAACTCCCCAACTCCTTAGTTTTCC
AAGTATGTAAATGTTTCTGCTCATCCAAACACTAGCATAGGTAAGTGAGGACACATATTTCATCTAGCCCAAATATATTTTTTAAAAGAATCCC
TGCAGATTTTTTCCTTCTATAAAAAATATCCCAACCCAAATTTCTAAAAATTTTGATTTTTGGAATTAAATGATAATACTAATTAGCATTTACC
TATACATCCTTTTCTATTTTAAAATCATTGCATAATCTGGCATTTATATTTAATTTTCTAAATCATTTTCCCTAGAGAGCATTCATGTAATTTT
AGAACTTGGAAGGAACCAGGATTTCAACTATACTCATCCACAGACTTTTCCCATTGGGTAGAAATTGAAACAGAACTGACAGAACCAGGATTTG
AATACCAGCCTTTTGACTCCAAATCAGGGACAAGATGCAGTTTTGTATGTTAATTATTTTTATTGGTTTTGATATTGTGGCCCCAAGATCCATA
GTCTTATTTTCATGCTAAGTGACTCTTTACTCTGAAGGGCCAGGTTATAATTGCCATGTATGCCAAGAGTCAACGGACCCAGGACCAGAAAATT
CAGTATGCCCGGGTGGTGAGTTTTTAGACTTATGGAGCTGCATGTTCTGAACTCCACGTTCTTTGGTTTTACTGCTCCTGTGTTTTTTGACAGT
TCCTTTTTTTTTTCTTCATAATTTCTGTACAGGCTGGGCAGAGAGTTCAGGAAATGTTCCACCGTGTACTGACATTCCCCTGGCAATAAGTGGG
TTCCCACTTCAGTCGCATTCCACTAAGCCTATTTTGGCACCATAGAGAGGGTGATGAGAGGGAAGAGTCAGTGGAGGAGAAGACAGGAAGGTTG
CCACCAAGGAATTGAAAGGCATATGCTCCTGGCCCCATTGCCTGTGTCTGCCTTCACCACCTTTAGCTTCACAAGCAGTGGCCATGGCAAATAG
GAAAAGGTTTGGGTGGGAGAAACACAGAAGAAGCCATTCCTCATTAGGCCATGTGTCTTCCACTAGTTTCCATCAGAGTGGGCTGACCGATCTG
TGAAGATCTGGGATCATGACTCAGTATAACAAGAAGAGTCCTACACATTTGATTCAGGAAATTTGGGCTCCTGGCCCACCCAGCCCTGCCTTCC
AATCTCAGCATGGCCTTGGACACATCCTTAACCTCTCTGGGCCTCACATTCCTAATGATAAAATGCAGGGACTGGACAGAGATTGCAAACTGGT
GGCTGACAGATGTTTGATTTGTCCCACACAGTATTTTAAACACATTTGAATTGGTTGCCAATATTTAAAAATTGGGCTATTTCACACACACACA
CACACACACACACACACACACACACACACACACACACAAATCCAGATTTCCAGCTCCCCTGGAGAACTCAGATGATCTGGTGTTTCGGGCCCAC
ATAGCTGCATGGAAAGACCATGTGGGAACAACTTTGAGTTCCGTAAGAAAAATCAATCAACTTCCCTCACCTGCTTGGCCTGTGTAGACATTTG
AGTGTGGGACCCCAATTCTCTCCAAGACTCCTTTGAAGCTCTAGTTTTAGAATTCCAAATATGATTTGCTTGACCATAAGAAATATACTTAATC
CATAGTGGTTTCTATATTCACGGCAGAATGCTAAGGTATTTGTTTATACAATCAGCATCAGGTAAAAGTTGGAACTATCTTAGGCCAAATAAAT
CAGCCCCATTTAGAACAAATGCTGACTTTGAAATTTGATAATGGGAGGCAGGAATAGTTCAGCAAAGATAGCCTTTGAGGTAGTTACAAGAAAG
ATGGTTTGGCTCCTGACTCACTATTACCTACACAGCGAACTGTGTATTTTGCAAATTATCCAAAAGTAGATTCTTTAAAACCCAAATCAAATAA
TACAAGGCCCTTCAGTTAACATTTTTTCACATCTCCCACTCTAAACTAACATCTTCATAAATATTGACATCATGAACTTTTTCTGTTCATGTAA
TTGCAAACAAATTATATTGGCCTGGGAACTGTTTGATCTTCTTCAAAAGTAAAGGAACTACATTGATTTTAAAAGTTATATTTTTGTAAATCAT
GTTTCCAAATGTATCCACTTTAAACACAAAAGGTAACATTTGCGACTCATGGTTATAGAGTGTTTAATATTGTACAAGGTGCATTCACAAAAAT
ATCTCATTTGATCTTTACAACAATTGTAGGAGCTAGACAGAGAAGACATTTAATACAAATGAGGAGTCTGAGACCCAGAAAAGTTGGATGACTA
TTGGAAAATTGGCCTATTACTTGCTTAATGTGAAAAAGAGCCGGGATTCAAATCTACAGCTTTTGGCTCCATATTTAATGTTTTCCATGCCATC
AGTATGCTTCCAGTCTCTTAGGGTATCTGTGTGTGTGTGTGTGTGTGTGTGTGTGTGTGTGTGTGTGTGTGTCTTACCTAAACACTGACTTTTT
CTCTCTCTCTGTCATGAACAAGAATTGAGAGGTTAAATGGGAATTGTCTCATGAATTGTCTCAATGTTTTCTAAGTTGAATGAAGAATCTTTTT
TGGTAGCTATTCTTAAGTGAATGCTGGATTTTTATTAAGGGAAGCTAACAGATATTTAAAAGCACCCTCCATATATAAAAGCACAATCCATGGA
AGCATAGTGCAAGGAATAATGATATAGACAAATGGAGGAGGGGCAATAAAAAGTCATAAAACAGGCAGGTAGAGGTTTCTGGAAAAATAAGGTG
GTGAGGGAAGAGTCTAACTGTAAAAGTAAAAAGGTAGAAGATAGAACCGTTGAAAAAAAAAAAGAAATATCAGAAATATAAAGAGAAATAAACA
GCAAGAAGACTCCGTAGTAATTTAAGAAGTGAAAACTTTTAGGACAGAGGTAGGAGAGAAATGGGAAACACTGGAGAGGAAGTAACAACTAGGA
AGTGTGGAAGAAAAACCTGCAGGCCAGAAATCTGAGCAAAGACTTGAGAGAGGATGATGGTAAATGTGAAACATTGAAAGGGATGTTTTAAGGG
ATTCAGACATTGAGAGGAAAGTAAATTTTCATCACAACACCCCATGACGACAGGCTCAACACATTTGGATTATGAGAAAGGAAATTAACATGGT
TAGAACGAAAGAATAAAAATTAGTAAGAGAAATCTTTGGATGTCAATGGACAGGAGGAGAAATGATATTAAAAGGAAAGAAGGCATGGGTGGAA
AGTAGAATGAGATGAGAACCTATGGTAGGTGGAGGTGAAAAATGAAATGCGTCAGGGAAGATCATGACTGGGACCGCCGCACACTGCAGTGATG
AAGACGGAGAAGCTATTCTTCAGGGAACCTGCAAATAAAAAGAAACCTTAAGATGTATTTGGAGGGAACCCACCCCAGCAACGTGAGAAAGCAA
GAGGTTACATTCTGCAAAAAGAGAAAATGAACCGGGAGAAGGGGGAAAACTAGGGGAAAGAGAAAACAGCAAATCAAAGTTATGCAGCGTACAG
CATTGAAAGGAATGAGGCATTCTGTTGGAATTATTCATTTAAGCATATGGGTATTGGCGTCCCTTTTCTAAAACCGGATCCTCTCCCTTATTTC
TCATTTTTCTATAACACTATTATAATAAATTCACATGGAAAAGTACAAGGATGAGCAATTTTTTACTTTAGAAATGTTTCAAAAGGGGAGGTAA
ACTTGATGGTTATCTGTCTGAAGGGTGACGCACTATAGTGGCATGTGAGAATGAGGCCAGAGGTAATCATCAGTTTTGAAATTGTTCTTTCAAC
AAATTCATAATGCATATAAAGTAACTCGTCCACACATTTATGGCAAAACATACTCTAGATATTAATTTATATTGCTTATACTGAAGATCTCTTG
AGTTTTCAAAAAGTTTGATTTACAGATTTTTCACATCAAAAATTTTTTGTAGATTCTATTTTGTAATTAAGATGTGTAGACTCCTCAATAATTC
TTTACCTTGATGCTGTGTATACTCATTTATCTAGATTAAAATACTTCCTTGCCCTGAATGTTCTAAAAGTGTACCCCTTGGGGCTCTAGAATTT
AGAAGTCTCCCTTCCCCAACTATTTCTAGTCTACCTTTTCAATTGAGTTAATATAATGGCTTTAGAAATACAATTTCCATGTTGCTATAAAAAT
GGGGTTAAAAGTCATAACAGGCAGAAGAAACAGGAGGAAATGAGAATTGTGTACTTTCAGTCACACCCGGGCCATGTGCTCGGGCCACATACAG
CATGCAGCCCTCCGGAGTGGGCTGTGTAACCCGGGGCACACTTGAGGTCAGGGCACATAGAGGATTAATTTACTGAAGGTGGTAGACAATCCCT
CTGCCACAAATGCAAACATTTTTTCATAAGCGCCTGAAGCAATTAGTTCTCTTCAATAATAAATTTAATCTTTTCCTCCTTCCTTTCTTTCCTC
TTCGTTTCTCCTCTTTCTTCTCTTTTCCTCTATAAATGCTGGCCTTTGTCTAAGATCTTCAACTCAAAGGTGTAGATGCCAGGAAAGAAAAACA
AAATCAATGTATTGTGGTACTAACAAATAATGCTATAATTATTAGAAAAAAACAGATTGAAGCAAGATACAAAAATTCTCAAAAGCAGCTATCT
GTATGAAAATCTTTTCTACTTCTAGAGAGTATGTGAATGACTGTATCTTTCATATCGTGAAAGCAGATGCTTACATGCAATCACCTTTAAAAAA
AGAAACAAACTATCACAGTAGTGACATTGCCTATTTTATATGAGTAGGGAAACTAGCCAAATAGCTTTGCGAACATTTATTAGCCTTGTCCTTT
TTTTTTTTTTTTAACTTTTAGTATAGAGAGAAAATAGAGATGACAGGTAGGTGGCTAAGTAAGGTGACAGGATGGCAGACAGCATTCCTATCAG
GGCAGAAAGAATTTATAAGGGAGCTGCCACTTATAGCGTAATAGAGCTAACTTAAGAATTGCAAACAGAGTAGGGCCAAGCAACATTTTAAGAA
CAATTGTCCTTAAACATTCTTGTAATGCTAAGTCACTCATCTTTGTTTCCCACTTGCTTCCTAGAGTTAAGAAAAGTTCTTTCCTAACTTGGGT
TGCTATGCAGAGATGTGAAATTTCATTGGCTCCAAATGCTTTTCTCTCTAGTAGGGACTACTGGGAACCCTGCTAATGTGCATATTGATGTACC
CTGTAGCTTCACAGATGATGAAAGAGCATATTACTTATTTCCACAACAACAAAGCTAAGAGAAATTTGAAAGGAGCCAGTTATTTTACCTGGGG
AATTTCTCCCAGGTGATGGGGCTATGGAACCATGGAATTTAAATATGGGGGCCGTCCTATTTTCTAAAAAGTTTTTATGGGGAGCCATAATATC
ACTCCCAAGCAGAAATCAAATCAAATAAAAGACTATTGCTTTCATTTCCATTTATTTAGTCAACTATGCCTAAAATACGGAAAAGATATTATCA
TGCTCATTCAGAGTGTATTTTATTCGTTGTTTACTAGACTGGACTGACATGATATGATTTCTGCTTTGCGGGGTCACAACAAGCTACTAGCAGG
ACAATTTGCTGAATCAAGAGGATGATTGTGTCTGGTGCCAGTCAGTCGCAATTAGGACCAGGATCAGGGAGGCCCGGGGCTTGTGCATCGTCTT
GGGCAAGCTGCATAGAAATCAGCTCACCTGAAACTAATTGTCACAACAGAGACCTTATGCAAATGAGCCCTGGGGTTAGCGTGGGAAAAGAAAA
ACTGTCGCAAAGATAGGTGACCTGCTGAGCAGGGGGTCTGTTGCTGGGGAAATCTTTGTTCAATGAGTTTAAAAATGTTTTCATTGTTCAAAGT
CCACCCCCCGCCCCCCCCCCCCCCAAAAAAAGTAGCATACACACTCACTAAGAGTATGGCAAACACACTCATCTAAAATTCCAATAAAGCAGAA
GCCTGCTTTGGAGCATGCCATAAATTGGGAGTCTTTATAAGGGCTACAGCAGGAGAATAATTTCCCTTTGCAAGGGCCTTTAAAGTTTAGGTGC
TGCGGATTGCAGTGTGGCCTACCTTGAAAATTCACTTGCAAGGTCAAGCCTATTGCTAGCCTGGCAGGACCCGCACAGCCTTTTCTCCCTGCAT
TCCAGCAGGCCACCTCCACACAGCCGTCAGGGGTGCCTGTGAACTAGACAAGAAAGGCCTAGTTTCTAATTGAGCTTTACTGGTATCTCAGGTA
GAAGAGAAGGGCCAGGAGCACTCTCTTAACACATGGCAAAAGAATCCGGGGAAACAATGGCAGTTTCTTGAATTGTGGTTTCTCAAATACTCAC
AAGTTCAAATGAAATGCAAGCATTGTGAAATTCCAGGAATGGTAATGCAGCCCAAGTGGTAATAGATCATAAAAATTTAGCAAAAGAAAAGTAA
AAATTATTTTTAAACTATGATTTACTTTGTTTTTCAATCCCTGACAAAAACACGTATAAGTTGGAAATGACGTTTGATTTTATTGTAGCACTCA
TTTGGAATAATCTTTAATATGTTTTACTTTAGAAAACCAGATGAAGAAAAGTTATTCAAAGGTAATGATAAACATTTTTATACGTTACTGTAGC
AAAGTTGCCTTTGGGTTCTTCAATTGATTTTTAGCTTTTCAATCTTGTTTTCATAAAAACATGGGCTAACATATTGATACTTCTTAGCTTTACA
CTAAAGATTTCCCAGTGACATAGTAGATAATGTATATTCACACAGGAGTATAAATATAATATCAAGGAAATGAAATAGAGTGTTGTAAGCTTTA
GCAGTAACAGAGAGCATAGATATTTTGCATCTAATGATTTCAAAGAAAGAATAATTTGAATTTAAAAATGTAGGTGAGAGAAAAAGAAAGTTTC
ATTGCATTGGCGTTGAATACAAATGCTATTTTGTTTGGCAAATTACTGGACCTAAGAAAGTGTTTTCATTGTGTTTCCTAAAGGGCTGGATTGA
AATTTGGACTTGTACCTGATAAGCAAGCTCAGGAATTAACTTGGTAGCCACCACAAAACCTAAAGAAAGTTAGGCTTAGAAGTGCAACTTAATC
ACAATTTAGATTAAAACACACACGCATACACACACATATACAACTCTTTTGTCCCTTCACCACTGTCTGAAATAATTTCCACATAAGAGACATT
TCACAGTGGGTACGAAGTGCTCAGCTTTCACTCATCAAACTTTAATCGGAACTGCATTTTAGTTTTTGAATAAAGAACACTTAAAAAATTTAGA
TGCAAATTATTTTGCATTATTCATGCTCTAAGTGTCCTTGTAGATTTAAACACTTCTGAGGCCACCTTGATTCAAGTAATTACCTAGGCCCTCA
CTGCTCCGTCATCAATAAGGTATTTTGTACAAGTAAAGTATGCACCCGATGACCTTTTAAAGTGGCTTTACACTGAACTTGTGTATTGCTTCTT
GTGCATGAGTGAGCCGAACATGTGTCGCAATCTGTCTCAGGTGGGAAACCCTGGCTGGGCATCCATGGGTGACGGGATGAAACCCGAGCGTCTC
CTTCACACACATGCTCTTCCTTTGTCTCTACCTAAGAATGTTCCTGCCTGATTCTAGTAGTTGTTTTAAGCATTAAAAAAAAGCAATAGATTCT
AGGAAAAGAGACAGGGAAACTGACAGTGATTCCGCTTAACCCCTTCATCACCAGAGATTATTGGAAATGCCCACTTTTCAGGCCTCCTCATCTC
CATCAGCAAAGGCTTCCTTGTAAGGTAATAAGTCTCTCTCATTTTACATGGTTCTGTACATCTGTCTCCCCATCTGTCAGCCCAGCATCCTGAG
CTATTAGAGCTAGGTGCTACCTCCACCACGGTATTCACACGAAATGGAGCAGGCTTCAAACTCAAAAGTACCTCAACAATACTTACAATGGAAA
TGAAAGTGACCATAGACCATCAATCTGTAAGTTTATCCACACACACAAAAAGAGGGTTTGTAAGAGTTAAATGTAACTGGCTGAAGCTTATAGC
TGGCGTTTGTTACAAAGTACCCAAGCTTTTCCTTTCCAACTTCGTGGCTTTGAAGAATTATAGGAATTTTATGATGTGCTGTTGGAGTTATACT
GGCAGAGATTACAGTAAAGGGGTTAAAGTTGGTTTATGGTAGAAAAAAAATTATGCAGAAACCAATAAAAATAAACACACTCAAGTAGACTAAA
CTTGCTGGGCTTTTAGTTTTACTTTTAACAAAGGAAGTTTAGATGAAAGTAAATGTTGCAGCTTTTAGAAAATTAGTTGACACTCATACTTGAT
CATCTTTACTATACAGTGGTATAATTAATATTTAAACATTCTAATAATGCAGCTTGCTTTGCATGCACTTTTCAGCCTCACAACATTATATTTT
ATCTGGAATTGTGCTAGCATTTTCTTGGGTAGTGCCCATTTTCATAGAGTTAAATTTAAAATAGGGTAGGGCCTCAGCTCTTATTGCCAGTAAA
ACATCAATAGGAGAGTGAACAGGTTTCATACATCTACCAGACAGTTTATTATTAAGAACAATATTTATTTGAGATAATAGATAATATATAAATT
TTGTTTCATTGCCCCATGAATGTTTTTAAATGATCAGAATAGATTAAGTTTGAAAGTTGCATTTGGCTGTTGTCCACTCACTGTTCAAAACAGG
AGGATTTTGATGATGTCAAGGCAGTGCGAAATCAGCAAGTGATACATGAAACATGGAACCCCCATGCCTGCCCATTCATTAGGGCATCTGATTA
ACATAGTAAGGAATGTTTATTTTCAGTACATAAAATTGTAGAACTTTGGGAGCATCTTCTGAATTGCAAAAGCTGAACACCCAGGAACTTAGTG
AAACAATAAAAATATAGAGAAGTAAGCTAATTTGTGAGAAGCCTGAGCTAGTTTATTTTCCTATGAGAAGTTTCTTCCTGTTCCAATCTCATGG
CTGTAGGTGGTAGGGAAAAGTTGTTTCAGGTGCTGCCCTCAGCTAGTCCCTTTGAAGAGTGCAAGGTGCCCAGTGTTCCTGATCTTCTAGTTTG
AATGATGAGAAAGAGCATTCTACCTTTCCACCATCACTTAAGTGTCCTTTGAAATTGAATTTGCATCCACATATTTACTCCTCATTCTTTTTAA
GTGAGTAGGTGTGCTCAGCTTCAAGTCTATTTTGAGACTCAGCCCATGTTAACCCCTTGAGGCCCACTTCATGATAGGTACTGCCCTGTGGGAA
TCCTGTCATTTAGTGAAGCATGAAGCCATAATAGATCCACAGCCATACCACCATTGGGGCATCTCATTTCGATTAATTCCCTGAAGCCTTTGGT
GCTGTGGTTTGCAGAAATCAAATGGCGCAAATGAGAACTACAAAATTAGGCAGGTTAAATTTCGCAAGAAAAAAAAAAGATAAGTAGTAGAAAA
AATAGCCAGTGTAAAATATTAGTACACAGGAGCAGTAAGTAAACTAAGTTCCACACTGAAAGAACCACCAGATCTCGAGAAGTAGATATCAAGA
AACAGTTTGCACTGGCTGTTTTATTTCCTTATGTCAGAAGGAAGTAGGTTTTTACAGATCCTTGGACATGATCATCTTTGTTTCTGATATTCAG
AAACGGAACCAACAGTTTGGTGTCTATTTATTTTATTTGGTTTGTTTTTGAGAAAGCGAGTTAAATAAACTCAGCTTACAAATAATTAACAGCC
CCTGGACTATTTAAAACCTGCTACTGAGGTGGTAGAGATAAGTTGAATCCAACAGCTGTCACATGTATGGGTTTGACAGATAGGTAAGTCCAAG
AGTGGACAAATTAAAAGCATTTTTCATATGCTAATCTCCACCTGCACAGGAATAATTTTTTTTAAGGAGGCAGTCTACTGGCAGAGCAAATTCA
AGAGCTATCAGTAGTACATTACTTCTAGGAATTATGACATGCAATAATGAATAAATACTTCTATGAGAAAAGTCTGCTGTGGAATCTTAATTCC
AGATGGGTGTCCTGACAGCAATGTGCCAGCAATATTGTGGAAAGAAAAATGACATATATACATACATATATATATACACGCACATATGTATATG
TATATATATGGCTTTTAAACTTGAATAATAATTTCTTCCATGTGTAATACGTTCACAATTATATCTAAGGAATTACTTTTTTAGTTGCGAATAG
AGATCAAAGTGAAAACAATAGTCAACCTTGCCCTTCAGGTGGCAGTTACATGTGAGCTGCATAGTGAGTGTGACAGACAGTCAAGGATGTCAAT
CAGAACCTACAGATTAACCCACAGCAGTGAAATCTGAGATGGTTTATAACAAGTAGATAACAGTTTCAGCCCTATGTGAGAACGCATAAACTAA
AACTTCATGCTGGGACAAAACAGGGCAGGACATTTGAGGTAGAGAGTGAGAAAACCATACTTTTATTATGACAAAGCCTCTCCCATACTTGTTG
AAATGTCACTGGGTTGTTATTAAATCCTCACCATTTCTCTAGGTTTTATATTATCTTACAAAAAGCTTTTGGAGTTTCCTTTAATTTTGATAAA
CATTCAAATTTGTGCATAATTTCTGCACTTCAGCTTTGAATGACAGAAAATTTCCCATGCTTGTTGAGTTTCCTTTCAACTAAGAAAGTAATTC
TTGTAATAAATAAAAATGTTATATTTCTTTCTTCCAATGTTATCAAGTTAATAAACCCTTACCTTAGTTTATAAAGTCAAAATTTACTAATGTA
TTGAAATGAGAACCGAAATACTCAAAAAGAAATCTTAAACATCAAATAATAACGAGGGTAATGAATGAATGGTCATGGTTATGGATGGTGATGA
TCTAATGTAAGGTCCTATCTCTTTTTAATAGAGAGTCCTCAGGCTCAGACAATTTAGGGTTAAAGTGCCTATAACCAGATCAGAATCCTCAGTT
ATTACGTAGTACTCTTTTAAGACCCGTGAATTAAGTTATAATCAATGATTACAACACTGGTATCATTTATTAATGTACCCTTCTCTTCTAGGGT
AGCAGAGATCAGTTTCTGACACCTTAGAACTCTTCATGTGCAAGAGGAAATTGAAACAATAACAACATATTTAGGCAGTAGGCAAAATGATTTG
CTTTGGCTTTACCTACAGGGAAACTTGGGCAATGGTCATTAGACTTTTTTAATGGAACTTCAAGTCTGATGCCTTGTGACCAGGTGCTAACATG
GAAAAGAGTTAGCATCATTGACATTCTTTGATTCGGAAGCACATAAGAGGTAGATAAGGAGACAACACAGGCGACCCACCTCCAGGCCAGGAGA
AGAGGACAGTTCCTGGAATGTCTGCAATGTGTTACTATTTTTGTCACACCTATGTGAGGCCATCCATCTGGTCTGAATCAATCAGTCTCATCCC
AGGGCTCTGCTCCTCTCCCAGCTGAAATAATACCCTTCTTTCCTCAGTGCTGTCTTCAGGAGGGGCGCTGCTCAGGACTGCCCGGACAAGGCCA
ATTAATGAGCCTGTGTATTTGCAAACGATGACCCAGGATTAGACACTGGAGAAAACTTTCTTTAAATTCTCTATAAGCTCTGCTCTCCTAGAAA
CAGTACTTGAGAAAACCGGGTGAATCATTAGACAAATAATTGACCCCAGAAAGCGAAAGACTGATGCTACATTACTTTGCATACGTACATCTTA
TTTTTTAAAGAAAATGTCCTTGCCTTGAATTTAAACCATGAATTGGTCTGCTGTTATACCCAAAGTAGATAAAGCCTGGGTGTGAGGCATTCAC
CACGACTTAGCCTGAAGTATTTTCTTCGGCCACTTCACTCTCCTCCACGCTACATGTTCAATTTCCCTGCACCTCTCTAACAACCCTACACCTC
TGCTTCTAGATCATCTTCTTGGCATTCCTCATGTGCACATCCTGTTCTCTAGCAGTCCCTTATCCATGCCGCCTCATCAGATTCCTGCACACCT
GACAGCCTCTTGCTTGCTATTTCAGCTACTAGACTACTGACCTCTATTGTACATGCCTGCAATCCCAGCACTTTGGGAGGCCAGGGTGGTAGGA
TGGCTTGAGCCCAAGAATTTAAGACCAGCCTGAGCAACATAGTGGGACCCTGTCTCTACCAAAAAAAGAATCAAGGTATGCACCAGTTAAGCCA
GCCACGTTTTAGGACCACATAGCACTCTTCCCACAAGAGACCTCAAAACTTAATGACATTGGCTTTGGCAAATTCAATACCATGACATAAAACT
ACTCATTTTCCCTATCTGCCTAGTTTTCCATTGCAGTGGGAAATACCACCCTATCGTGGACCTCCCAACCAGGAACCCCATGGTTATTTTTGTC
TCACTGCTGTCTTTCTCACTCCCCACAACCAGTCTGTTACCAACCGTTGCTACTTTTTCTTTGCCAATGTTTCCTGAATCCACCCCTTTCCTTT
CCATTCTCATAGTTGTGGTCTTAGTCTAGGCTTTGGCCTTGGCCCATGGGGACTTTGCAGCCTTCTTTCCCCTCTCCCACCACATTCACATATC
AAATTCCAGCTTACTTTATTTAATAACACTGACAAATTTTTTTTTCTCTTGTCTATGTTTTTGGTCCATTCATTCTCTCTGAGCTCCATTGAGA
CTGCCAGCTGCTCTCTGGACTGGCTTCTAATGTTCACTTCATACATGTCACCTCTTCATCCAGCTACCTCCTAATTGTTCCAATCTTTTTTCTT
TCTTTTTGGAGGTAAGCTCTTCTGGTACTTCTTTTTTTATCGTTCTCACACTTCTTCAGAAACTCTTCTCATGGTATACATGTTCCTGGTACAC
ACCACTCAAGGGCAGATTCTGACACTTTCTTTCGGTAGTCCATCCGTGCTTCCTTTCTGATGTAGCAAAGTGTCCTCTTTGTGAGCACGCCCAG
GTTCAAAACTTCCATTTCCACCACTTGCTACCTATGACATTGGGCAGTTACCTGACAGCACTTCACCTCAGCTTCCTCATCTGTAAAATGGATT
GTTGTGACGAGTAATAAGATCATTTATTTAGAGCACTGCTGGCACACAGTAAGCACTGTTAACATGTTTGCTATTACTATTATTATCATATCTC
ATAGGACTCCCCAATCACACCCCGCTTCAATCACCATCATGCTGTATGCCATATTATCCTTGCATGTAAATTTTGAGGACCACATAACTGGGTT
TTAAAAATCTGGTTCTAACAGTAGTTTATGTCTTAGAAATCAGGGCAAAGTTAATCGAAGATACCTAATATCAAACCAGGTAATGATGAAGACT
TTAAATGCTTTTTGAGGATGATTATAAAGAACATTATGATAAGAATGATGATGATATGATGATGATGATGATGATGATGATGATGAAGAAAACA
TGACCAAATGCTTCTTTGGGTTAATACTCTGCTTATAAAACGAAGTGTCCAGGAAACATACAGTATTCCTCCTGAAATTAGTTTTTTTTTCCCA
TTAAATGGAAACATGTTATGCATAAGAACATATATTGTGCAGTATGCATGAGATTTATTTTTCAGAAAGCAGAGAAATGGCAAGAATTTTAAGT
GTTCTACATACTTATTTAGATCTCACATCAGGAGATAAAACAAATGAAAACTCTGACTTGTTAATAATTTACGTTTTGGTGGAAAATTCATACA
AGCTGTTAAGATACCTCTAAGACCCAACACAGAAAATGCAAATAACATATATCCACTGTGCACTGAAGAAAATTTGTCTGAAAAGAAAATAACA
TCAATTAAAAAAAAAAAACACAAGTGATTTCAAGGGAATGTTGACCACATTTACAGGGCTGTAATTGTGCTTATTAATTTTAGATGTCTCTTTT
GAAATATTCGTGTAAATGATCTCTCCACTTACCAGGTGTTTCTTACAGAAAGTCCACTGGGCTACCTCACTACCTAGGAGGTGGCTTACAAATA
GGGGATTTCTTTTTAGAAAGCCACACCATTACTTTTCTCTAACAGCTTATGCAAAAGTTTTGTGGGGGAGGGAAAAACCCAAGCCAAAAAAAAA
AAAGAAAGAGAGAGAGAGAGAAGAGTGTGCATGACAATAACTACCTCTGTCCCTTAGGTTTTTGTTCTTTTCATGTCATGGAACTTTTTAGCTG
TGCTGCCTGGGTAACTGCTCTGGAAGGTTGGAATATCTGTTGTTGAAGGAGGTTTGTGATTGAGGGCCCTCTGACACAACCTTTTGTTGCATTG
AGGTGTTTGATCCTCTTTCCCTTTCTTCTAAAACCAATCCCATCCCTTTAAAAAATGGATAACCAGCACTTCTTTTACTTATTGTCAGCTTTTG
GAAAGTTACTCTGGGGGTTTGTAGAAGTTTAACTAGTTAACTCTTTGCATGCTGATCTCCTGTCGCTAAATATTCTTCTTCTTTCTAAACAGGA
AGGATTTTAATCACTTACATCAGGTAACTACCGCTGTTATGCAGGCATAGCAAGTGAAACATATTAAGGTCCAGAAATAAAGCATTTGTGTTCA
AGGTCTTCAAATGTGAGAGTGATTCAGGCTTTGCAATGAGACACATCCTTATTTTCTTTTTGTCCTAGAACCGAAACATGTGAGAGTTATGACT
CTGCCACTTGAAAAAACATGATGATATACTAATTTTTCATTGAAGAACAACAGGCTTTGGTAATAAGTAAATGCAAAGGGTTACTTCAGAACTG
GGGTACATAACTGAATGGCAACAAAGAAAGAGAGCCTTAGAATGAAAAGTGTAGGGGTCTTGTTCCAGTCTGCTGACTAAATGCTGGAATATTT
TTTTCCCAAAAGAAGGGACACAACCCAGGCTCTAGGAAAGAAAAGAAGCTTAAGTGGATTATTTAACAAAAAAGCAACTTATGGCTTCTAGATA
GTGACCTACATTCTGCACACAATAATGATGTTTTACGGTGGTGATAGGGTTAATAACCTAACATCATTCATGTAGTGTTTTTTGAAAATTGAAT
TTTACTGCTACAAAGTAAATGTAACAGCCCTCTGGAGACAATATTACAGGCACTTTATTGTTTGCTAAGCTGATGTCATCTTTCTACAGACTGA
CTTTAAAACAAAGGCACAACATTTATTACAAATATCACTGTGTGAAAGGAAATCTACTGAAATATTGTTTAGAAGGTGAGCTGATAAAAAAGTA
TTAATACTTGGCTTTGATCATCTTGTACTATAAAGTTATGACTTCAAATTTTTTCTCATGTGATCTCTATTCAAGGCAAACTTCATCCTGGCAA
TTCCTACCTGTGTCTCTAACATTATTTGCCCTCAAGTTGAAACATTTGTTTAAGATTCATGCGTGTTTTAAATACATTTTTACTCTCTAAGGCA
ATTTCTTAACAGAGTTGATGAGTTTTTGAGAGAGAGAGATTAAATAGCATCTTTCAGAGATAATATATGTATGATTTTAAAAACAAAGTATGGA
GATTAAGCTGTTTAGTTTATATAATGAACTTTCAAAATCACCTGTAAGTGGTGACTGCAAAGTTTGCAATTTAAATAAGCTGTTTCAGGCACTG
CCAAGCAAGCATTCTGCATCACAGGTTGCTTGCAACTCAGAGGGTTTGGTCTCTGTATCTGATGCACGTGTAATACAGCGTGAAGTTGTTTGTG
GATCACACAGGAGCTTCTTAGAGCTTCATTTCCACACTATAACCCTTGAAAATGTATCAAATGAGCAGATGCACTTGGCTCTTCCTTAAAAGGC
ACAAGTAATTTGTTTGTAAATCAGTTACCTTTCAAGAAAAGTTAAATTAGGGGGAACTCTTGTTTCTTTGATGTTTTCCCTCTTAGAAAGCCAA
AGAGCAAATTAATGATCATTTTGAAGTAATAATGATAATGAGGAAAATATAATTTAGTGAGCTACACTATCCCAAAATAACTTTTTCAGGTACA
GCATATAAATAACAGCCCAAAGGACTAAAGCAGCCTGATGGTGTTTTTCTAAGAAAATGATTTACTGCTCTGCCTTTGTCTCTGAACTCTGGTT
AGAAGGAAAACACAGGTCAAGGAAATTGGGTTAAGGGAAAAAAAGCCTGTTATGGGCTACGTGTAGAATTATATGAATAGTGACTAATTGGGAG
TCTGTAGCACTCAGATCAGTGTGTGCAATGCTCAGTACTGGCTGATGGTATTTATATCAGTCAAAAATGAAGCTGGATACATCTTTGCGTGCTC
TATGAAGGCAGTGTGTTAGTTGGTGAATTGGCCGTGACCAGGAGAAAGCTACCAGTAGTACTATGATTGTGACTAAACCAAGAACTGAACTGCA
ATGTTTTAAAAATTTATTTTACTAATAAGTGTACTTCTCTATACAAAAATCATTTTTAAAAAACCTTACAACTATTTTGAAACATTTTACTATA
AGCCTGCCTTTGATTTGTTTGGGAAGCTATTTTATAGTCACTCAATTCATTTATTGTATTTGACAAATACTAGCTACTTACCATGTGCCAGCAC
CAAAGGTTCAAAAGTGAGCCCTCACAGAGCCTTACAGATTAGTGTCAGAAAGAACTGCTTGGCAAGGGTAAGAGTGTGACACATTGTGAGTATT
CAAGAAATGTTTGTTGAGTGACTGATAGAATGAATTGAGGCATCATCAAGGTAAGTCCTCGATTTGGTATATCTACAAGGACGAACTAAGAGTT
TCCCTCCCTGAACAGGTCAGAAAGGACATGCTTGAGCTGGCCTTAAAAAATCACCTGACTGCCATGTAGCATGTGAATAATGAACACCAATGGG
CCACTGGTGGTAGAAACAGCATCCCAGAATATGCTCTGAGTTTTAGCAGCACAGCCCATTTCTGACCCCTGCATGCCTCAAAGCCACTTTAAGA
ATTTGGTCTTTGCCCTTCTGTCCTCACCCCCGTTGTACTGTAATACACGCAAACATGTGTGTGCATGTACACACACATGCACACACACATGTGC
TAACTCCATCGTTGGAATTCGACTGTCCACCCCTTCAAGAATCAAACCATCTCCTAAAAAAAGGAAATGGCATGCCTGTTCCTTACTTGTATCC
ATAAAACTCACATTTGAGTTGTCTATCATGAGACCTCAGTTTTTTGACCAAACATTGTCCTTAAGCATCAGACACATAAGAAAAACTGGAAAGA
GGAACAGGTAATATTTAAATCACCCACTATTCTTGTAATGAAAGGATTTTTCAAACATCAAGATGAAATGGTTTTATCAAATTATTAAAGTTTG
CTTTTTGTGTCCGACTTAGCAACAAGTCATGGATGGGACACTCAAAACAGGCCAATTTGGGACAGTTCCAACTGTAGGAGGCCTTGTGTCATTA
CCTCTATAAATGGAATCGAGCTTCCCTTCTTCTGCAGCTATACATTTTCTTGCCTTTGATTTCCCACTCTGCTTTCTCTCCCCGCAGGCTCTTC
CTGTCTCCTCTATCAGTTACTCTATAGGCAGATGTGCCTCTTTTTCCATTTGCCAGATCCCCCCAACCCAGATCTTCCAAACTACTCCCCTTTG
AAGTCTCTATTACCGATACAATCACTTTTGTTTCCCTTCCATCCCCTCTCTCACTGCTCCAGTTTCTTCTCTCCCTAACCCCATTAGTTACATC
TGCTGCCCCTACCATTCAGCCTGTGTTAAATCGTAATGCGCTTGACATAACTTCAGTTTGTCTGCTAAGTACTCAATCAGCACGCATTGCTATC
TTCTGATTTGTAGGAAAGGGGGCAAGACTGAGTAAGCTGGCTCAGAATTCTCTGAGTGATTCAGCTCTGCCCAATTAGCACAACCAAAGTGGGA
AATTGACAACTAATAATAATGAAAGAAATGGGTTTTAGTTGTGAATATAAATCCCACAAACTTGGTTCCTGATGCCTGTGGTAGATACTGTATG
CTTCATATGGTGGTCAGATGGCTCAGACAATCCAGAACTGGATGAATGAAGGAGACTGTGTCAATAATTGCAATATTTATTACTTGACTGGGAT
TCTTTCCCTGGAATTCATTAATGGATTTTTTAAAGACTCTGCAAACCACCAGAAATTGTATCTAACATTTACTATATTGATGCATTTTTTTCCT
GGGCAGTGGGTCCATACGTTTAAGCAGATTCTTAAAAGGCTCTATGTCTCCCCACTATCCGCCTCCAAAAAGGTGAAGAATCATTGCTATAAGT
GAAGACACCTATTTGGTCTATGTTTTCATGTGGTCTGCTTTTTTTCCCAGCCCATTTTAACATTCCACATGATTTGAGCAGTTCAGAGAGTAGA
AACGTAGGGTTTTAAAGGCAAAATTTACCTTCTTTAAGAGGAAAAATGTTTAAACCACCTACCAGGTTATACAATTCACATTTTAAAAATTATT
TTAGTTTTACCACAATTCAAAGACAGGCCTTTTTTTTTTTATTTAACAATGGGTATAATCACACATCTGAACATAAGAATTAGTTTTATGCAGT
TCCATAGAAAACCAAAATCTTGAGGAACACTTTCAAAACAACCAGAGAAAAATAAACCATTCCTTATGTCAGTCTTGTTAGGGACGGATTAAGA
AACTGGCAATTATTTTGGCGACCTTAATTGTTGCGCGGCAGGAAAATATGTAATTAGAGCACGTTCCCAGGCCTGCACACCTGCAGCACATCCT
ACAGCATAATGTCAGCTTTCAGTGCCTGTCGGTGCCAGGTTCTAGCACAACCAGTCACAGTGACGCTCTGAGAACATTTCGGACTCATGTAATG
TGGCTTCTGTGTATGAAAGAAGCAGGCATTTACACTACCACACAATGGAGTGCATTGCTGCAACACTGGTTTCATTATTTTCTGACGCCTACCT
TCAGCACTGCCCTGGTTTACTCCGTTTGAGGTTTTATTGTTACGGTATAATAGTCCTGTACATTTTTGAAATTCCCTTATTATTCAGCTTATTA
ATACAGTACTGACTTGATTATCAGGCTGCAGTTGCACAGCGGAAGATTCAGTGCATTAGCTAAGTCAGAAGCTGAGTAAATTCTTGGTATAATG
AGAACAATTGTTTGACATTTTTTTATGTAGAAAGATGGGCCATGGTAGTTCTCTATATAGTTGCATTTTGGAATTCACTCTATATTCCCTATAT
GTTGTTGAGTATTCGTTTTTCATATAAAGAAAGCTTAAAATGTGTTCTCACTAATTTGAAAAGTGTTTCAGGGTATGGCAGGACTCCATCATGA
TTCTCATTAGCTTTTTTCTTCTTTTTCTTTTATTGGCTTTTCTCAACAAAAAGAAACTCTGACGTTAGAAAAAGTAGTGAAAATGAAGAAACAG
TATGCACGATGCCTATTTTTCAGATAATTCCATAATCTGGCATAGTGATAGTTAACAGACTTTTCCCCCCTAAGGGAGATACACTGTATTTTGC
TATCTGACATGTTGTCATATATAAAGAAATCAATTGGTAATTAAATGATGCACCTTGTGAAGATCACAAAAGGCATTTGGTTGCGAAAAGAGAA
CTGGGTTTTGAGGGGTTTGTAGTGAAGGCCTAATGACTATTCATAAGAGATTTTCAGTCCGTTGATCCACTTGACTGGAAAGTTCTGCCCTGGA
GAGTTAGTGCTGTCAGTTTCTCTCACCTCAGAAAGCCGCCGCAATCGACGGTGTAGAGGATGAGCGACAAATTATCTAGTAGTTGCTCTAGGCC
TGTCTTAGGTCAGGTGCAGAAGGCACATCTGGCTTCCTGTAGGTACAACCTCAGGACATAAACTTTGACTAATTTTTAAACTATAAATATGTAC
GAGTGACACTGAATAAGTTACCTTTTATTCATATTTCCTTTATTTTAGAATGCATGCAAGTTGTGTAGTAAGCTGCCATTTTGGCCTTATTTTT
CTTCCAATTTTAACTGTTACACACTTGCTTGTTAAGCTAAGACTGACTGTTTATGAAAGTTGTCAAGGACCAAATGATAACAGTCTCCTTATGT
GAAATCCAACAACTCTGTGTTTGCTGAGAATATGCCAAAGTTGCCCTGCAGGAAGCTCACCTGAAACAAATACAGAGTATATGATTGGATAGCG
CTCTTGGGGGGTGTTTATTGCCCTATTTGGCAAAAGGCAGTAAGATATCAAAATGTACATACATAGGACTGGATACAAGGCTGCCACCACATCA
AGATCTCTAGAATTGCAGAACATTGTTGCTCCAGTGTCTTCATAAACACAATCACAAAAACTACGATGAAAATTCATGCATTCAAAACAGACCA
GTTACTGTCCACCATGGACCAGGCACCATCCTAGCTTTGGAGGAGACAAACATGAGGGAAGTAAAATCTCAGTGCTCATAGTTTACTGAGGAAC
AGACAAATAAGAAAACAGTTACACTACAACATGTAAAGAGTTATGATTGAGTTGTATAATAGCACAGAGTTTAGAATCCAGGAAAGGGTTTTCT
GAGAACTGTTGGGGTCTACAAACGGGGTAAAGATTTCTTATACGAGCTACTCTTTCCATTGCCAAAGCCAAATTAGCTGGCGGAAAGGTGAGTT
GGACAAGTGAGGTGTTTCTGTTGCATGTGCAATCTCAACAGAACAGTAGTGGCCAGCAATTTCTGTACATATTTACTCTTGGAAAGTGCTTCTC
CATGTTATCTTATCAAGAAGCTCACCTAATCAAAGGTAAACACATTTATCGCCTCATTAAGGCTTGTTTTATGGCCACTTCGATGTCATGTTTG
AGGTAGAAGTACCTGTCACAGTCTTTGTCTGGAATCTATGTAAAAGGGATTTCATGGACACTGCAGAGGTTTTAATCTTTCAATATCAAAATAA
TTTAATGCATAATATGTATTTGAGAAGATGTCACTGGTGATTCCTACTTGTGTACTGTGTGTACTGAGTTTTGCTGACACATTTGCTAGAGCTG
TCAAGGTGGACCAAATGAATGGACAGATGCCAAAAAAACAATTATGCTTATTAATGTACATTGATTGCTTGTTTAGGTTATAAAACAAATTAAA
AAAAACCATTAGTATTTAAACCCTTTTATAGCTGAGCTAATGATTGCTTGTAATATAGTGTCTGGTAAAGAATATTCTATAATACCTGTAAAGC
CCAGCTATGTTATCTAATTAGTAGACTTTGATTTAAGTGGCTGTAATTATGGAATTGAATAGTTGCCTTTCTAAATAACCAAATGGTAGATTTA
AATATATAAAGATGTTGACTTGATTATTTAATATTTGTAGCAAAGACAAACCACCTAAATAGAGAGATTAATTCCACATCAGTGCAACATTAAA
AGGGAGAGAGGATGCCAAGAATCCCATTAATTTGGGATATAAGTGTCTGAAATTTCCTCAAAACATTTACGCCAAGCAATACCAACAAGACAAT
ATCAAGCAGCTCTCCCTAAGGATAGAGAGGTAAGGCAAACATGATTTTAAACTGCACAATATCACACAGTAAAATTAAGGACAATGTGTTCCAC
CAGATGCTAAACACAGCCATTTATCGAGCTACAAGGTTCTAGACTAAGTAAGTGTTTTGTATACATATTAGCTCCATATTACCTCCATGACCTC
TTTCCCCATGAAGCCTTCCCTGAGCCTCAGTATCCACTCTTTCCTGTACCATGAATCCATTTTTCTTTTACTTTTTCTTTTCTTTCTTTCTTTT
CTCTCTCTCTCGCTTGCTCTTTCTTTCTTTCTCTCTCTGTCTTTCTTTTTTTCTTTTTTGTTTTTTGTTTTTTGTTTTTTGACACAGGGTCTCA
CTCTGGCACCCAGGCTGGAGTGGAGTGGCACAATCATGGTTCACTGCAGCCTTGACCTCACAGGCTCAAACCATCCTCCTGCATTAGCCTCCCA
AGTAGCTTGGACCACAGGCATGCACCACCATGCCCAGATAATTTTTTTTTTTAATTTTTGTAGAGACGGCGTCTCACTATGTCACCCAAACTGG
TCTTGAATTCCTGGGCTCAAGTGATTCTCCCACCTCAGCCTCCCAAAGTGCTGGGATTACAAGTGTGAGCCACTGCACCCAGCCCATGTTTTCA
ATAAAGATGGTGCTATTTGAAACTATGGCATGTCACGTGTATATTATTTTATTTCACCATGTGTTTAAATGAGATAACCACCTGCCAAGAAACC
TTCAATATTTGCTTAAATAGCCAATGTGACGGAGAATAGGTAACCTCTTTTCTTTTTTTTTCTTATGATTTGCTTTGGCTATTAGTTTTGAAAC
ATACATGTTATATTCCCAATTAGATCTCGTTACATCCTTCACAGGCTGTCCAAAAATTTCCATACTTCTAACAAGAACTTCGGTTGACTGAGTT
TATATTATATAGTTTTTCTGTCCTTTGCCCTATTTCTTTCAAGGCCTTTGAGTCAGAAGCATTCAGCACCACGGCGTACGATAGCCACCATTTG
CTTTGCCCTTTTAACTTTGGGGGTTACTTCTGACCATAGTCCAACTGCTCAGAAGGGCTTTTTCACAAATTGTTTACCGTGGCAGTTGCTACCC
TATTGGGGTTTGCATTCAGAACCCATAAGCAGAGAGCAATCGTGTCAGTATAACAAATAATGTGTACAATTTTTTAAAATGCTGAATTTACCAA
ATCCCCATATTATGTTTTCACACTGCTCTTTCAGAAACTGTGCTAATACAATACTCCTATAAACTGATCTTGGTCCTGGCAGGCCCTGTTGGAA
CTAATGGGGCCTGACAGTTCTTCAGTTAAAAATTATACATGAGGGCCACTTTCCCTTAAATTATCTATTGTATGCCAGCTGTTCACAACTGCAC
TGCTTCACTTTCTTTGCAGTGCTAGAAATGTCTAATTTTCTCATAATATCCATAGCAACTATTCTCATGGAGGGCATTGAAGTTAGTAAGACTT
CAAACACACCTTCACAGAAATCATGCTAGAGTTGCAGTTTCCATTTTCTTTAATTTTTTGGTTCTGAAAAATGTTTAGATGGCTCATTTTGCCG
GTTTCATGCCATAGCACTTGAGTTATATCATAATTTCTCATTTGCTGCACAACTTTATTCTATTTTTATGCTCCTAAATGACGCAGCTCTAAGT
ACCCGTGAGTGGCACTTCCGTGCTGTGAGTGCTATCAAAGGCAACCTCAGATGGATAAAACTCCTGGTTTTTTGTTTTCTTTTGTTTTTTGTTT
TGTTTTTGTTTTGTTTTTGCTGGGGAGGGGAGAAGCGGAGTCATCACCAAGGGCCTTCTTTTGTTTTTCACGCATGTTAAAACTGTATAACCAA
CAGAACCATTTAAAAGGAAGAAAAACATCACCCAAGTTCCGTGTCTGAATGAAGGCTGTCTTCTCAAATGCTCCTCATTACTCCAGAGCAGTTA
TATTTGGATTATAATTAAAACGAGGCTTCTTTTTTTTCTTTTATTCTTCTTTTTTCCCAGAATGCCTCAAGTGAAGTATTCCTCCCAAAGTTAT
TGCACCTCTTTACTAACAAGTGCTGGGATTTTAATAAGCCTGTCCCCCCTCCTTACCCTGTACAGAGGAAAGGATGGCAAAGAGTGGCTGTGGT
ACAAAGGTTACATATTCAGTGATATTTTCTGCTTTTATTGGTAAAAATGGGGAGGTGGGTGGAGGATTTCCTTTAATTATGAAATAAGTGTGAT
GTTAGTAGTTATTTCTGTAACCTGGTGAAAATGCTGCATATTTCAGGCCTAATTTTTAGCATATGTTACCACGTGTGTAATAAAACGGTGATCT
GCGCTCTCCGAATCTTTTGGCTGTTAAGGAGCACTCTGATTATAACTAGTTGCTGTAGAAACCTGGTCATCCAGATGCTTATCAAGAATGCATT
ATTATTTTTTAAACCTTGGTGGACAAGCCACAGACTGCAGCAGTTGGACAAGGTCACATATCGATGGAACTGACATGTCTTTCCTCTTTCCATT
GTGTGATCGTCCAGGGCCTGGGGGCATTAATATGCAACGGTTCTGTTTTTCTTTCAGAAGGACTTGTAAACTCTTTAAACAGAGGCACACAGTT
GCAATTGTTGCGGTACTGAGTGGGTGTATAGAAGCTAGGGCTGCAGCCTTCTCCAGTTGCAGATAGAATAAAACTGACAATGATGCAATGGGCC
TGGCATGTAGACAACTCTGTGGGATGGATGAATGGTGCGGCTCTCGCTGTCCCTCCGGTGGTGGTAGGCCCTGTAATAGCTAGCGTAGGAAGGG
TGGTGAAAATTGATATGCACTATTACTACACGTATAAAACTTAACACTGGGGCCATTGCACAGGTGGGGGGTTCCTGTTTATTGATTTTTATGA
GTTTGTTAGAACATGCGCTTAATGCTTTTATACTCCGGGAGCTCTGGGAGGGCTCACCTTAGGTAATAAACCTTTCCGTACTTTGCAATCGCTC
ACAATTTACATGGGGACTCACATTATCCAGTACAAAGCCCAGCATTCCCCTTCTACTTGGTTTTAGGAGTGTGATAAAAAAAAAAAAAGAAAAA
GAAAAGAAAATAACTATAGCACTTACTTTGAATTTTTTGAAAACCTGTCCACTCCCGCTATGAAAATGGCCCCTAATTCTAATTACTCATATTT
CCAAGAAAAATTAGTCACACAAACTTATGTTATGTGTTGTCTCAGAGGTTGAAAGCAAATCAGGTTTCCAATAAGACAGTACATCAAACAAAGT
AGCATGTTCTTGATGCCGATGACTTTTTTTTAGAATTATGGTTGGACATTTTTGGCAGACAGATTGTCTTTTGCTCTATCAAAATGACAGTTTC
TTTTGTAACTGCAACATTCATAATTAGAAGGAGAGAAGCAATCTGCCCAATTTAATATACTAAAAGAAGTTTAGAGTCTCTGCTAGTAATTTCA
TTATTTTTAAATTTGAATTAATTGACTTAAAATAATACAGTTAAGAAGTATAACCAGCAGGATTATGGAGAAAGGTTTCTTTTTCCTTAAAAAA
AAACAGTGCTCTCAGTGTTACACTTAAGATGAGATTGAAATTTTCTTTTTTACGCCAGGTGCCCCGTTCTAAGATTCAGAGCAGCAATGTGAAT
ATTCATCCTTGCAGTGGAGCTGAGAGAAGTTGTAGGGCAAATCCTGGGAACAAGGTCACTTTAAACAAAAAAGTTCTCCAAGTTTCCCATGCTT
ATAAGGCTGGCTGGGCTTAATTAAAGTCAATGGATGACCAAAAAATAAATAAATAAATAGAGAGAGAGAGAAAGAAAAGAAAATGAAAGAACAA
CCACCCACCCCATATCAGTCTCTTTCTAGGCCTCACTTCACATTGTATACCTTAGCCGTTGATGTTCAAGCTCTTTACATAGATCAAGAATAAT
ATTTCTTAAATCCCTTAACAACACTGACGTGTCCATGGTTTAATCACTCTTCCCCAGTCCACATCTCTAACCTACGTCACTTGGGTGGCCCTCA
CCCACAGCACCACAACAAAAACAAAGCCAGAATTGCATTCAGAATGAAATCATCATAACATAGCAACCTTGTTCAGGATGTGACCTTTGGGTTT
CAGCAACCCTGTCTTATGCAGCCCTGCCCAACCTTAACCTTTCCTGTTGGCTCGGAGGGTAACCTTTCTTCATATTTTCTTCAGGATCTCTCCT
TACGGGACCCAGTATGTTGCATGCCTATGATGCACAGGGCCAAAAGTTTCGGAGTGTTAAAATCTACGAGCCTGTTTTCCTCTATGAGACAATG
AAGCCTGCACTCATTTTAAAGAATACATACGTAGCAAGATCTCTTTTTACCCTTCCTGTATAATTTTGGTTATGCTTAACTCTAAAGGAAACCA
ATTCTCTTTAGCTCTGTTACCTTATTTCGTACCTTAGCTGATTGTCATCTTTACATGAGCATCAGATATGGATGTGACAAAGATTTTTTAGAAA
TAATAACGACCAGTGCTTTATGAGTTGTGAAGTGTTTTTCTCTCCCACAAGAAATTACAACACAAAGTGCCCTATGCATTTTTTTAGTATGTTG
ATAAGACAGCAGGAAATTTTTTAACCCTCAAAACTTTCTGATTATAAGCTGTGCTCTAAATTTACACGGGCAGTGTTCCAAGTCCCACACTCCT
TTACAAGTATATCATTACAGACCTGCAAGTGAGAAGGTAAATTTCCCCATGGCAGTTCTGAAATCTGACTTCAGGCTAAGAGGCCATCGACTGA
GAGGGCTGATTGTGCTGGGTACCTACATATCAAAATCTCCTCTAGTATTTTAGTAGTTCCTTCTGTCCTTTTTGCAGCCCCGTGTCCTACAGAA
GTGACATTTTGTTCCATTTTCATTTATCTTACTCTCCTTAGTTGTATATCAAAAGATATACACATTTATATAACCCAAGAAGCCAGGGATTAAA
GCACAATATTTCTTAACACAGCTTCCTCTAAGATCTCCAGTAGCTACCTAATGCTTAAAGCTTGGGCTGACGTGGGTTATGGGGTTATAGTAGC
TTTATAGATATTTCTATTTGTGTATGCTATGTTTATATATGTAATACATATATGTGTGGCTTAGGGCTATACTGACATGTTATTTTCTATGTGT
TCATTTATTATAAACAGCAGGCAAGCAGACGTTTCACCTCACACCTCACTCTGGTCTCTATCATTCAAAAAATATTGCAAGATTCCCATTCTAA
ATTAAATACATCCGAGCTTACGTCTTTTATCAATTCTTTATTCTTGGTATCTCAAAATTCTGGTCTTCGTCCTTTGTCTCAAAAAGGGAAACTG
CTGAAAGCATTTCCACCTGGATGTTTAAATTATTTAAGTTCCAGGAACCAGACCTTTTCCCTCCTGAGTAGTTGTGGGTTTTAAAGGCTAAAAT
TGCTTCATCATGCCAAAGTGAAAGTATGCATAAATTATTTAGTTAAACCTGAGAAAATACAGACTTTTCCCAGTGATCTTTCAAGGGCTGCCTA
CTTAAATTTCAGAAGAATGGGCAACATTTTTTTTTCATTCTCTTAGAAAATTGAAGTATCTCTTGTGTCTTCTGTCAGTTGGAATGTGAACAAA
ATTACAAAATGGTAATTTTTTTAAAATTCCAAGCTTCATAATATCCAGTATATCTATATCATTCCAACGTGAATACTGTGATAATCAATCAATA
CCAGGAAGACTCCTCCAGCTGGCGAGAACTGCAAACCCTCTCTCAATGTATTCTTAGGAAAATCAGGCAACTTGTTATAGGTCTTTCCCTAGTG
CCAACCTAAAGACTGGGGGGCATTTCCCATTCAATTTCTAGTCATCCAGAGTTGGTATTTCAGTTTTAAAAGTAGAAAGTTAGGTAATTTTAAG
ATGTTATGAAAACTTTGTTGACATTTTTAATTTGCTTTTTAAATTTACCTTTACTTCAGTAAATAAAAATACCAGAAAAGTGTGAAGTTGTGTG
GTGGTGGTGGTGGTTGGGGTGTGTGTGTGTTTGTGTGTGTATGTGTGTGTATAAGATAGATAATATTAAAGTAGCAATGAAAACAGCCCAGAAA
TATAGGCAATTGAGGCCCAATTTCAAGTTTCTGTAGCATAAGGCATGTATATTACAGAATGTACCACATTATGTATCTGATCTCTAAATTCGCC
TTAGTATAAATGCTCCAAGTACATTCTTATGACCAAGAAAAGAATGGTATTTTTAATTGAATACCTAACCTCAAATCCTGAAAGAAACATTAAT
TGGGACAGGACAAAATGTCAACAAAAAAGTACTTATGTGAGTTATTTAGTATACAGCTGTCTGTTCATTTGTAGAATCCATTTGAATTTTATGT
TCAGAGAAATTGGGAGATGAGAGTAAATATGGGGGGAGATTGGCATGTAAATTTGTTGAGTTTTCATGAAAAAATAAAAATTTAAAAAACCTTC
CAAGCAAAATAAAACCTGTTCCATTCACAAATGATGGATGTCACTGATCCCATTAATACCTGTTACATACCAGGTCCTCTTCCTTATATCTAAC
TTGCTTTTTCTCCCAATTTCAGTGTAGTTGCTCTTGACCTTGCCTTTGTTCTTCTCTCCTCCTTCTCTCTTCTGCCCAGGCACAGTTATGTCCA
CTAAACCAGATGAACAAGATTTCATACTGACACCACCATACTCTGTTAAATAGTTAAAAATCCTATTTTGGCTTTTGTTTGCTCACTTAGATTT
GTACACCCAAGGTGATGAAGAGAGCACAGATAGAATCTAGAGACAAATAGTTTCTGGAGTCAGGAATCAGATCCAGATTCTAACACGAGGTGAT
GTGGGAAAAAGTCAGTTAAAATTTCTGAGATTCAATTTACTCATCTATAAAATCATCCTCATAATAATTGCTCCCCCCTTCAAAGGTTTGTTAT
GAGGAGCAAGTGGAAAAATGTCTATTAACGGGCTCTGCTGTCAGTAGATTAATGCATAAATCCAGGTATTATTATATAATCAAACTGTCCTTTT
AAATGTAGAAGAAAAGGGTTTTTAAGGCACACATGAGATTCTGGAAACGTACACGTTATAGAAAGTCTATAGGGACTAAAAATGCATTGTCTTC
TATATGCATTTATCTTGCCAGAGGTACATGCAGACCTTTGGGGGGTGCTGCTCTGGCCAGGACAGTGGTGGAGAGCTCTGACTCTTGAGTGTGA
GAGCTGGAACGATTGAGAAAAGACAGGAGGGAAACTTCGAGGTCAGGTGACTTCTTTCTAACTACAGCAGGTTCTCAGATCCCTAGGCTGGCCC
AAGATGAGAGAACAGTCACCAAAGAGAGTCTGGCACAGTAAGCCTAAGTCTCAGTGTCATCTGATTGTGAAATCTGCTTGTTTGCATACTCATA
CAGTACCAGAGCTTAAGCCCTGGCCACTGTACGGATGACTGTACAAGAGCAGAATCTAAGACCAAGGGGTTGTTTTTACTAAGGAAGAAAAATT
TATTAGAGTTACTTCAAGGAAATCTATTAGATTATCTCACCAAATACATTAATAGCATCAATTATTGTATTTTACATAATTTTTTTTACACCAG
ATACACAGTATATTAAAGAATTTGTGGAAGTCCCCCATGTGAACCAGTATTAACCATGATAAGAGTAATCAGGACTGGCCCAAGAAAGGTAGAA
ATGAGAATTTATAAACTTGTCTAATTCTCATTGCTTGAAGAAAATTGAAATGAAAGAATCCAAAAAGGGATTTATGAATATTTTTTAAAGCTTA
AAAAGCCCACATACTTATCATCGAATTGTCTGCTTACGATAGGAGCTGGAAGAGTCCCTTATATCCACTCAACTGTACTTACATTTTAAATGGT
TGTTTTCCTTTCTCTTACACTACTACAAGAGAAAGGAAATGCCTAGGCAAATAAACTCAGTTAAAATAACTTTATGGTTGTGACACAGTTTGAC
AAAAGCAAAGGAAATTCAGAGTCACTGCTCACACTGTATCCTCAGCTGATTCCAACTGTGGAAAATAAAATGCTTTAAAAAAAAAAAACAATGG
AACAAACTTGGGATGGATTTAGTCTTAGTCTTAGAGGCAGCCTTCTTAAATGCTGAATCTCTGAGTTCGCCATCTTTAAGTTCTTATAAGAGTT
GCAATATCTGTATTCATCATAAATCTAGTCAGAGTTAAAGAAGAGTATTTGGTTAAGTACTGCTTGGAAAATATGTTGTCCTTTTTTAAGATTA
TGTGATTCTAACTCAAAGCTGTAACTTCTTGGGGGTTAATAACCTACCTTGAGATATACTTTAAGTAATAATAATGGCTTCTTGGGATTAGGAA
AGATATTAAACTTACCCATTTCCAACGATCCTGTACTATTGCAGGAGAGATGTGGGGAGGGAGAGAAAGAGAAAGAGAGAGAGACGAGAGAAAC
ATTTTGACCAAAATTCCATATTCACTATTGCTATGCAAATTATAAACCTAACAGAGAGGTGATGTATCATTTTTATTAAACTTATGCTGCACAA
ATAGTGTTTACATAGAAGAGTATAAAGTTAATTCCCTCCCGGGAAGCATTTCTAAAAAATATAAATTGTATTTTGAGAGTTAATCAATTTGTTT
TTTCTCTTTTGGCATTTTAATTGAAGCTTCCAAACCGTATGCTTCCTCAAAGTATCAGAAGGCAAATATCAGTCGTGTGGTTATAAAATCAACT
CTTAATTTTCTTTCAATGAAGTAGCAGATAGTCAGCCTGTAAATTTTACCTACATTACTATGGAGCTGCAGTGCTATAAATTCAAAACAGTGTG
CAGGTTGGTGGAAATTGAGGTCCAGGCAGCAAGGCATGTCCATAAACTTTTCTCAGGTTATTGGAACAGAACGAATCAAAAGCGGTTCCTGGTT
GTATGATATTCTCTTTTGATAGAGCAGGCTGCTTGTTGTTAGCAATCCAAGTGCAGAGGTTAGTCAGAGCAGAATGTAATTTCTAGCAATCTGA
CAGACCTGCGGATTGAAGCGGCAGGTTTTCCCTTCCGATTTCAGGGAATTGCTTCTTACTGTGACAAATGATTAGCTGTTATCTCTGACTGAAT
TTGATTTCTACAAGCAGCTGAGTCAGGTAACTTTGCAGCGTTTGAGGGGGTTTTTTACAGGAAATGAAATCGGCTCCCTAGTCATATGTGGACC
AAATGAAATCTTCTCTGTCAGGGCAAAATTGTGCTTTTGGTTAATTGAAGGCAGATTTAGCATAGAGGGTGTGTGTGGTGATGTGTGTGCAGGG
TGTGTGTGTGTGATGCTCTGATGGTGTAGGCAGCCACACCCATGTGTGTACATTTACAGGAACGAGGGATCATTTCATGGATTCTCTCCCAACT
TTGAAAATCCTGGGTTCAAACAAAATCCCTACATTCTGAGCAGGGTTGAAGCTTTTAGAATAAACTATAAGTGGAGACAGTCATGTGGGTGTCA
GAATAATGAGCCTTCATATAAGGACATCCTGTTTCAAAAGAGTTTGAACTGCAATATTAATCAAAACACAATAGATACTCACTGAGCATGGTTT
TCACAAAAGTATTCATCAAAGGGCAGCTGCATTCAAAGCAAGAGATTTAGGTAAAATAAGTTAATCCTTGATCTCATCCAGGTTTGTGTGTAAA
TTTTGAATAGGGAGGGGTTCTAGTGATAGCAAGCGTCCTCTGGATGATAAAACAAAAAACGTGGCATGGGAAATTCCAGGTTTATCTTGTGCTT
CTTGTTCGCTTTTGATGGAATCTTAATAGTGACTACACAATTAGGATCAGGTTTATCCTTCTGTTTATAGTTTGTAATTGCTCTGGAAATCCTG
AGTTTCAGTAGTTCAACTTTTAGTTTTTAAGCAGTCGGTGAGTTTTTTAAAAAGTTACTATCGGATAATGTTTCATAGTTTCTAATCCTATTAC
AGCAATGTAAAATAAACTGTATTTCCCAAATACGAAGCAACATCTTTTATTCCCAGACCTCTTACTTTGCAAAGAGGAAACACCATCTTTAAGG
TCATACTAGTAATACTCCTTATTTTGTGATGAACATAATATACTTTGTTTATAAATCCTAGTTTAAAACAACAGAGATTTGTGGTAATTGACGA
AGAGTAAAAAAGTCACAAATACTGAATCTTCATTTTTCAGATGTGAGCACAACATTCCTAGGTATGTACTAATATATTCTAACACGGCATTGCG
CTGTCATCAAGACTTTTGTCTAAATGTCACCACTATTATGAGCAATACTTTTGCTAAATTCTTTAGTTGTATTCCCTAAATGGCAGATACCTTT
GGACCTCTCTCTCATTAGCTATTAAAAAGAATAGTTCTTTGAAAATAACAGTTCTGTGCTCCCTAAACTTCTTTTGTTTTTTGTTGAGTGATGT
CAACTTAGAGTTCCACACTGATTGCCCTTCCTCCTTGCTTTGTCGCCACAAAAGGGGTGAGGGGGTGCCCGAGTTTGACCCACACCTCTGGTTA
GTATTAATCACACAGACATCAGGTAATCACCTAAGTGCACTCAGACATACTAATAGAATGATTTACAGACCAGGCCCTTTGTCTTTTTCTAAAT
CTCATTTTAATTTATTTAAAGTGACAGTGAAGCTAGACTCCAAGCTAATGAGTCTGCATAAAAATGATTTCTTTTTCCACCTTTGTGTTTGCTA
AAACTTTCTGACTCTGAAATCCTGTCAGTGAAAAATTATTTGAGATGAAACACTGCAAAAGAAGGTAAGGGGAAAGGAGACAAAGAATATGATT
TGATCATCAGAGACCTGCCCTGGGTCCAAGTGGGCCTCATGGTAGCCACAAGTGTCCAGATCAGACAAATGGGGACTTTTGCATATATGTTTTA
GTGCTTCATCCAGACTTGAGTGCTTCACAGAGTCTGTGAGGCAACTCACATCCTTTGGTAGCTAACAAATTGGTCCTTTCTGACCAGCAAGCAG
TTTAAGAAAGCTAAGCTGTGTTGTATGCGTATTAAATATAGCACGAACTGCTTGGCTTTGCTTAACCTTAGTTGTATTAACAGATTGTTTGTTT
TGATGACTGCGCCGTTAGCATGCCAGATGATAAGGAAATGGTTTTGGATTAGTTAGGACCTATAATATAGAAACTAAATAAGATGATAGACTTA
CAAGGATATACATTTCCTGTAAAAAAAAATGAATTCCCTTATCAGGATTTACTTGAAAGGAATATGTCTTATTACAGAAGGAGAATGCCTTGAG
GAATGTGGGAAAGATTAAAGGCTTTTGTCAGTAGTTTAGGTTGGGACCAGGTTTCCTGTGTAGCCAGCCCCCCTGTCCCCACGTAAATGTTTTG
CTTTTCCTCCTCTCTCTCTTTCTCTTTAAGGGGAAGAGTAAGCCCTTCTCGCTGCCTGGCGGTGTCAGATTAAATACCGAGATGGCGACAGGAA
AAAGGCTGGAAGGAAAAACAAAAGACAGGGGGGTGAGAGTTATCAGGTTGGCGACGGAACTGCAGGCTTGTCGGACTCCAATTTACAGAAATCC
AAACAGATTTTAGTTGACACGATCTGCAATCATTACCGCCGGAGCAGAGCTAATTAAGAAATTAGCTTTCCTGATTGCCTGTTCTCACAGTGAT
GAGTAGTTGAATAATGTTGTATGGCTGAAATGTTCTAAAACCAGACAGTTTAACAAGTGTTTAGCCTTTTGTGGTGATGCTGTATTTACTTTAT
CTATAATATCTGTGCTGCATCACTATTTGTTTCCTCTCCTTAGAAGCACCCCTTGACTTTTTTTTTCCTTTTCCTTGGTTCACCCTCTTCTTCT
TGGCATTAAGTCAGAGCTTCCCCCCTTACTTCAGCCGCGTATAGGAGAGAATTATCATACAGATTTGTGGCATGTGGTACAGAAAAGTTATCGG
AAACTTGTGGCACATTCAAAAGCATATTTCCTACCCGTCTAGGCATTTTTCACAAGAAAATATCAGGTCCAAAGTGGCATTTGAAGTTTCTGAA
TCTGACTAAATGCAGAGGTGATTGTGAATAAATAACATTAAAAGGCTTTAGTCAGCAAAGATACAGTACGAGTGAGTGGAGCTCATTAGAAACT
AGCCGTGGACCCCAGATATGTCAAAGGGGGGTTTGCTGAGAGGTGACCACGCAGTCTTCATAACTTCCTCATCTCTCCCACAACTATCAAACAC
ATAGGAAGATGTTATTTGGAGAAAAAAAATAGTTATGTTTAAAAAGCTTGACAAAGAAATTAATCAAAATGAGCTTCATGGACCTTGTAAAATA
TGGTCCAGGCACATGTAATGCATATGTAATAGGCATGCGTACCGATGTGTCCATATGGACAAATAGAAAACCCAAGAGTTAAAAGTTTCTGCAA
AAAGACAACAACTTCTTTCCAAATTAAATAGAAGGCTTATTTGAATATAAACTTTCTCGCAATACAGCAAAATAACAAAGATTGGACAAAGGTT
TCCAGGTGTCACTGTTGAAACTGCAATGGGAGGTAAGAGTATTGCTAGCTTCCCAAATGAACCACAGAAGGGGAAGTTGCATCATTCAAGATGC
TTATCCTTTAGGATTTCCAAGTTTTAAAATGTAAATATTCATCTATGGAAAAACCAAAATTTCCAAGTTTTCCTAGTACACCTTCAAATCATTT
CTACACCTAAGAAAAATAAATACTAATTTTTCTTAATGAGGCCTAATAACACCTTTAACTTACCATTTTAAGAGACGCTAAGTCAATACAATGT
GCAGGTCCCTTCTGGATATTTATTTTCCTAAGCACAAATTCATTTATACCGAAAAAACAGTGGATGAAATACTAGAAAAATCTTTTCTGCACTG
GACCTCTCTTTTCCAGATTTACCTAAGAGAATCTTACCAGGAATGGAGATAGACTTTTACTTCCTAAGTAGATTTAAATTTAACACCAATTTAT
TCACTCTAGTATTTCTATATGGGAACTAACAGCTTCTATAGGACCTCTACAAAGTGCCAGAAATATGATCTCATGTGTAGTAATAAATTTAAAT
GTGTTTTCTTTTACATATTCTATCCCTCCGTATTTTCCCAATCTCTTCCTTTTTTATGACTCCCCCACTCTCAGTTTCTAAAATCACTTGTCCA
CTCCGGAGACCCAGGGGCACTGCCTAGAGAAGGAGCTAACTTTAAAAAAAATAAACCAGGCTGGTCACAGTGGCTGATGCCTGTAATCCCAGCG
CTTTGGGAGGCCAAGGCAGGCGCATCACCTGAGGTCAGGAGTTTGAGACCAGCCTGGCCAACACATGGTGAAACCCCATCTCTACTAAGAATAC
AAAAATTAACCAGGCGTGGTGGTGGGCGCCTGTAATCCCAGCTACTCGGGAAGCTGAGCCAGGAGAATTGCTTGAACCCGGGAGGCGGAGGTTG
CAGTGAACCAAGGTCATGCCACTGCACTCCAGCCTGGGTGACTGAGTGAGACTCCAGCTCAAAAACAAAACAAAACAAAAAAAGCACCATACTC
AGAGCTGCTATAAGTAACATGCAGTTGTAAGCCACACACTTCTTTGCATGATTCTTATTTTAAAGATTGGAAAGTAGAATTCGTATTTGTTTGT
TGTACTAATTCCAGTGGTATACCTGGAATTAATAAATGGTACTAAGACACTAAGGCGTTACTTTCTTGCATTATTCTAAATCAGAGTGTTGATT
AGAGAGAATAATCCAAAGGTAAAATAGTCAGATAATTTATTTTTATGTAAGTTTAAAAAGAGAAAGAGATTGATTTTTTTGAAGAGTTATAATT
ACATTCAGTATGTACCTTTTGTGCATTGAAATAATTTTAGATTTTCTGAAAAGGAAATATTGATGTAATTCAGCTCTCAAAACACTTGATGCCA
TGCCTTTCTTCCTAGTGTTTATGTAAAAGCAAGTGGTCAATTGTCGCTGCATCAGAGCTATGACAAGTTCTTCTATATTAGGTGGGCATCACTC
AAACACTTTTCCATAGTAGATCCAGTGGTATACCTGGAATTAATACAATTTATTAAGTACTAAGCTATGTTGTATTTATTGTATTAATTCCAGT
GGTAATACAATTCCAGTGGTAATACAATGTATTAATTCCAGGTATACCTACCACTGGAATTAATACAATAAATACAACATAGCTTACTACTCAA
GTCTATTACACGCCATCTAAAATAGCTAATCAGAAAGGCTGTATGTCTGAAACACCCAAAACTCACTCATCCTCTTTCTTAAAGCATTAAAAGT
TTTGGCAACTTTTCTGAATCCGAGTTGTGTGAGTCCTATACTGTACTAAGCTGGACATCTTAAGGGCTGCAGGTTTTAATATTTACACTGCCCC
GACTCAGTTGGTAACCGCTAGCAAATCATCTCACCCCAGTTTTTGTCCTTTTCCTGAAGAAAGCTGAATTTGGTTTCATGATCACTGTTTGCTC
AAGAAGACAGTCCCAACAATGGTCCAAAGCAAGGCCAGCTTTTAAAACAGCCACCAACTCTGAGTCAAGGCATCTACCTTCATTCTAATCAAGA
AGGGAAGATACAAAGTTTCAAGACCAGTCTTCACCTTGCACTGAATCTGACGGGGTCTTACTCAGCCACTTTCCACTACCTTTCAACCTAAAAG
CCAAGAAAAGCAAGGATTTGGATTTTAAAGCTGTACTTGTTATAGGTGTTCATGGGTTTTGTTAGCATTGTTGTTTTTTAGGGATGAGGGGTGT
TACTGGAGTGGAAGGGGGAGCTGTGTTGCTTTTAGTTTGTCATTTAGGATGTTAAAGTTAGTATGGGTTTCAGTTTGCATTTTAAAAATTAGAA
TGGTAATGAGCAAGGTGCCAGAAGGAAATAACAAAGCACATTTTCCTGAATAGCTCCCCCTGTCTTCTGTGTTCAGATTCTACTCTGACTTCCC
AACCCGGCTTCCTCTCCCGCCTGCTCCCTTCTGAGGTGCAGTGTAGAAGTTCATTCTCAGGTTTCTCTCAGTTTGCAGGACGAGATGCTGCCGG
CCAGGCCAAGGGCAGTTATGCCAGCTCAGTGTAGTGCAGCTTCCTTGCCCCAGCGTGCGCAGGCTGAAAGCTCAGCCAGTTTCTCTTTCTGAGG
CTGCTTCACGTCTTCTCAGGGATTTCCAAGGCACGGACGTTCATTTGCCTAAAACTCTCTTAAACTGCCTGACAAAAAAAATAAGTACCTAAGT
ACGACATAAGTGGTACACCCACGCAGGATAATTAGGATTGATGTGCTCTTTTAGAGGCACCAGATCCTCAACCTCCCCTTACTAGTGGCATTTC
TCACCTGCTCATGTGGTGCCCACCTGTGGCTTGCTGGCATTGCCACTGCCTGCGAGTGGGGAGATTTCCAAGTTCAAAATGAAGGCCTGATTTT
ACATGGTGGAACGCTGTAGGACTGTGTGGAGCTCTGTGAAGGAATTAAGGATGGCATGAAGTCCTAGTTTATGTCACTGCTCCCTTGTTTATGT
TCTTTCCTTGACTATTCCCACGCCCCCTCTCCAGATTTTGCATGGCTTTTATAGCCTCTAGTAATTATATCAGCAGCATGGAGTCTATTTTTAT
CTCTTCTGAATGTGTGCAACGTAAATGTCAAAAACCATAAAGGCCCTTTTTAGCATGTACCTTGCATGCACAGTTTGAAAAGTGTGATGCAGCG
TTCAGAGAGATGTTGCATTTTGAGGTGAGACAGATGAAAAGTGGAAAACTCAATTTCACCACTTAGCTGCGTGAACTTTCAGTAAGTCCTTTAT
CATCTTGAACGTTTAGCATTTTCTTCTGTAAAATGGAGTCCGGCTGCCTACCTCATAGGGTGGCAGCATGGAACCTAGTTTAGTGTCTGGGATG
CCCCCTTTCCACAGCAGGTGCCCCCAAACTCCTAAATTATATATTTCATGCTTAAATGTCATGACCTTTCAATGCCCTGCATTGCCCTTTATCC
AACTTAGAAACGGAGTAAAAATAAAATCTTACCACTGTAAGTCACAATAGGATTGTCACAAAATTAGCATGTCAAAGAATGATACGGAACTCAC
TGTCACCAAAAAACTGTCCCTAGACTTGGCTAATAATTATAATAATACCTAGCATTTATTACTAAGCACTTTCTGCCAGCTGGTCTCTAGGTGC
TTTACATGTAACCTTCACAACCATCCACTGACCCAGTGGATAGATATTCTTGTCCTCCACTTAATAAACGAGGACATGGAGGCAGGTTAGATAA
CTTAACAAAAGTCACAAGTTACGAAGTTAGTCAGCTGGATTCAAATTGAAGCATCCTGATCCCAGAACCTACAGAAATATAAAATGTCTATCAG
GCAGGAGCATTCACATGGCTGACCTGCCCCAGCTTCTAGGCAAGGTTGTTTCCTCTCTTTGCACTTCCTATTTGAAGTTGGGCCCTGAAAGCGG
CTAGATGGGCCCGTCAGAAGGGCATGCGAAGCTGCAGTCCCTTACTCAGCTACGAGTTAGGGACCATCATGCTTCAAAGGGGAAATGGCCTCAG
CATGTTTAGGTAGTCCAGTACCATGGTACAGTTTTTATGGCTCATTAATTTATCTTTAATTGAAGATAAGTTGGGAGAATTTAGAGTTGTGAGA
AAATGTGTTGTTTTCTAGTCAAAAATTCTCCTGTAGTTGTGATGAATGGACAAGATGGCAGTAACTGGCGGATGTGGTAAGGGATAATAATAAA
TAATAAAGAGCATATTTGTAGGCCCTCAACAAAATCTAGTGATGGAAGGAAGAACATATATAGGCATGTTAGACATATGAGAAATATCATAATA
CATTTTATGGGTAAGCTAAGAAGAACCAGTTATGCAAATTTTCTCTACTTTTTTATGAACTACTTATTGATTACCCCAGTCATTTTTCCATTGC
TACCACTGCATAAAGCAGAGGAAGCCAGTGCAGTCAGAACCCTCTTATTTATACAGTAAAGGCTATTACCGACCAACTACTTAGCAGCCAGAGA
AATCTTGCCTGTAAAACAGAACTGGGATCTGAAGAACTTAGAATATGTCATTTAAAGAAATATGATCACTGGTATGTACTAAAGTGCTCCAACT
TTTTAAAAACTTTAAAATCAGTTGACTGAAATTTACAGTTATTAAACTTAATTCTTTTTTTTTTTTTTTTTTGAAACAGTGTCTCACTCTGTCA
CCCAGGCTGGACTGCCGTGGCCCGATCTCAGCTCACTGCAACCTCCATCTCTTGGCTTCAAGCAATTCTCCCACCTCAGCCTCCCCAGTAGTTG
GGACTACAGGTGCAGGCCACCATGCCCGGCTAATTTTTGTAATTTTTGGTAGAGATGGGGTTTCACCATGTTGGCCAGGCTGGTCTCGAACTCC
TGACCTCAGGTAATCCGCCTGCCTCAGCCTCCCAAAGTGCTGGCATTACAGGTGTGAGCCACCGCGTCCAGCCAATTGTTAGTCTTTGGTACGG
TGCCCATGAGTCATCAAACTCATATGTGGAAGTTTTTGAAAGAAGAAAAAAAAATGAGAGTAGATTGATTAATGACTATTAAAAATAGCCTGGG
ATCTACGAATACCCATAGTAAAGAGCACCAGGCACTGGATAGATAATTGGTCTAAAGAGCCTGGACCTTGCTGAAGATAATATATACATGTAAC
ATGTTTTGTGAAAATGGAGTCCTGAAGATGAAAGGTGTATCTTTTCCATATTGACTACGTGACTTAATTGCTCACTTACTAGGCTGAGAAAGGG
GAGCAGAGGGGATGTGGAGAGGGAAGAAGGAGGGAAGGAAGGAAAGAAGGAAGGAAGGATAGAGGGAGGAAGCAAGGAAGGGAGGGAGAAAAGA
AGGGAGGAAGGAAGGATGTGGAAGGACGGAAAGAGGGGAAAGGATGATTCAATATCCTTTGACTACTAACATATGGCCTATTTGGGGAAGTAGA
CGATGGTGCATGGAGTCTCCTGAAAGCATCATATGAATGTTTGAAAATGTTATTGCCCACACAGTGCAAGTGCATTTAGGTATGTTTTAAGATA
GGTGGGCTAACTCTGAACTAGGGATTCCCAAGTTTAACTGCCTTATGAAGGTTAGAATTTGAGCACTTAAGTTCAGGGAAGGAAGGAAGGAAGG
GGGAGAGAGAAGAAGGGAGACGGCACTGCAGGTAGAGTATCCCCACATGTCTTACCTCTTCTGAAAGGCTGATGCAACAAATGCGACTGAAAAG
CCAAAGTATGGATACGTTTTCAATTTATTTCTAAAGAGTTCAATTTAATAACTGTCTGAGACTGAAACCATTCACACAGCAGAGCTTCTAGTAA
TTATAATTCTGAGATTCCCCAAAGTGGAAAGTCCTAGGAATACTGCCTGAAATGACAGTGCATTTGCTGTTCAGGTAAAATTCTTTAGGAATTT
TCTCTGATTCTGATTTCTATTTCTAAAATTTTCGATCATCTTCACCATTTTCCCCCAAAAAATGAGGGGTGCATTTCTGTCATTTTGTGTGTGG
TTGGGTGGTATTGTTGCGTCTCAGTTAGGGGTTGTGAAATGGAAGCTCTGGAGGGAGGAAAGGCATTCTTGGTTTGCAACTGAGAGCAAAAGGT
ACCACCTAAGCATCTTCTGTGAGACTAGGGTGCTTCCTTTAGATAAATGTGTATCTGTAATCAGCTTAACTAAGCAGGTAAGGTGTTCATTAGC
TAGGCAGGGTTTTCAAAGAGAGAGAGAGGGAGAGTGAGAGGAAGAGAGAGAGAATGAATGTGGAAGAGTGAGAAGAACAAACAAGTCAAAGCAT
TTGTTCATGCTGCAATCTACACATCAATTATCCCGCTGTGCTAATTACCACCTTTCCATACCCCAGGTACCTAAGCATTCACACTTACTCACTT
TCATGCCTCTGATCAAAAAAAAAAAAAAAAAAAAAGAGGAGGAAAGAAATGATAGAAGCAGGGGATGATGAACCCAGAGGAGAAATACATTCTT
AGATCAAAATTTCCTTTACATATGAAAGTATAAATAAGGGGTCTTCTCCATCTTGCCATTGTTGTTTTACACAATTAAATATTTGCAAATGTTT
TAATTGCAAAATATTACTTACTGTCAGGCTTCCAGGAGTGCTTCCATCTCATCAGCCCTTGTTTCCATACTTAAAAGAAGACAGAGAGGTGAGA
ATTGCTGGTCTTGCCTGAGATTTTGGGGTTGTGCCATTAGAATGAGATCAGATCATTAAAAGTCAGTGACTACTAATTATCAGGCTACTTTTCT
GAGGCTATTCAAGAATTGGGCAGTGAGGAGCCAGAGAGCTACCTTTGTATCAAAACCTTGGTGTTCCCCCTCACCGACTGAACCTTCATCTAAC
AATTAAACATTCTTTATTATGATGAAATCTGGGCTTGGAATCCCTGTTGGTTTGTGGGCTTCCTTGTCAGGGTGCTCATTTTCTCATTTCAGAG
TGAAGTTGGATAAAAAGAAAACCTATTTCCTGTAGCTCATTTGATACCTGGTGGGGATGCTATTCAGGTGACAAGGATACAGGCACAGAGGGGG
AACAGATGTGATTTACACACTCATTATCATTTGAATGAAAGTGCAGATAGGCCTGTTCAGGGGCCTGGGCATCACCTGGACAGCTTTCCTTCTG
ATCTTTATCCCATCTGAGAGGCCAGGTCCCCCTGACATCTTTAAATCCCCGACCCCCAGCTGCTGACACACAGAGTTTTATGACAGTCAGCAGT
CCTCTGAAGCTCCAAGCACAGATGCCATTCAGAATTGTGCTTCACACTGTTTTTTTCATTGTGTTCAGGCTTAATCAGGAATATATCCAAAAAA
AGCTTAACTTGAATAATTTCTTTTGGTACTTCTCCAGAAACCTGTTTGCCACTCGAAGTTTGTGTATAGTATTCTAAAGGGCCTCAGATTTTGT
GTTCTTAAAGTTATGTGCATACGTAGCTGCTTCAGCTTTCGAAAAAGGACAACAAAATGGAAAGGACCATAAGTTATAGATGTCATCAAAGTCT
TATGTTTCTACGACTATCAGATCAGATGAGCTTAGTATTGTGGTAGATGCAGTCAGAATGTTAGAGTGCAAAGACAATAGAAAGTTTGGATGAA
GCTACCAAAAAGAAAAGTTTCTGTTTGGCCTCTTTTGCTCTTCATAAAGGATTTTCAGCGTCTTTGTTTTGCTACCTGGAATGGCTGCTGTCAC
CATGATCTCTCAAGCATTTTGTTACTTATTTGTGGGATGTGTGAATAACATGGAAAAATTGCTATGTCATCTGAAGACCTCAAATGATGAGTCC
TGAACAGTACAAACTGTTTTTTTTTTTTTAAATACATTTTCAAGAAAGCTTGCTTTCGTTCAGGACAAGAATGCACAACAACCTTGAATAACTT
TGTTTTTAACCTTCCCTTATCTCAGGCAGCCTGACAGTTTTGGTGGGGAGCGAAAAGTGAAAGGAATCTCTGAGAACAAATACTGTGGTGTTTC
ATGATTTTAATTAGGTATTGTATTAAAGAGAAAAATATAATGAACACAAGCACATACACATTTTATTTCCCATCTAACATTAACAGTTAAAGAA
AACAATCACTATCAAATTACTTTGTTACAGTCCATAATTACAACTCAGGTTACGCTTAACTGTTATTAACAGAATGTACAGTAATATGTTCTGG
CATCGTAATATGTTATTGTAGCATTATAATTAATCTTACAGGAAGTATAATAAATTTACAGTATCAGATTGAAGCAATACTGCATATAGATTTA
TTAATGCTTTTAATCAGTTCTGGCAAAATTAATTTTGCACTGATTGCTTACATTGGAATTTGCATATACTTAGTGAAAGTAATTTATTTTTTAT
GGATTCCTTTTTATTAAAATAGGGCATATGGCCCTTTAGCTAAAAAACTTTGTGGATTCTTTAGCTAAAAACATTTCTAAACATTTTGAAATGC
CATATATCTTCCCTTTAAGATAATTCTTTCTCTTAAGATTTATTTTTAAATTATATTCTTTAAAATACTTTTTTAACTCTTATTTCAATGATCT
ATTTTTTGAAACTGTAATATTTATTTCCCTATTGTCATTAGCCTACTTTTCCCCAAGGATCTAAGTTTGAATAAAAACACAGGCCTTGAAAAAT
ATCTACATTGATAAGGGACTGCATTAACATTTCAGAAAGTGTCATGTGAAAATTAAATATCATTGAAAAATAATGAAATTTTACTCTTTGAAAA
GTCCAGTAAAAATGGGACAGCACATGAGAATTTTGCCATTATTTCTAAGTGCAACTATGTTACAATACTCCAAAATGCTTTCTGTGCCACGCAA
TGTATGTGCAGAAAAAAGGCGAGTGCAGATTTTTTAAAAAAACTTAAACTGATATCAGTGTTGTAAGTTCAAAAATAGTATTGAGAAAAATATA
CCTAAAGTGTCTTTCTATTAAAATATACATGCTTTGTAAGGAAAGGTAAATTATTCACCAAAAAAAATCAAGATGTCTTCATAAAATATATGTT
TACTATATCTTAACTTGTCAGGCAATTTGAAATTTTGAATAAACTCATCACTTAAATCTTTATTTTACCTCTTTCAAAAATAGTAAATTAGGTG
TTCCTGTAGGTATTGATTAATTTATTTTGCAAAAAATATTTGAAGAATAAGGGAAGAGTGATTTCTTATTCTTATAAGTCTTAACAATAAATAT
GTTCCTTGGCTTGAAAATTGAGTGAGCCTAACAGTACATAGCTTTCTTTTCCTATGTTAGTGAGTTACATTATGTAAAGCATATTCATGAACTT
ATATAGATTCTGGGTAGTTCTGATGATATGAAACTAAATTTTTTAAATAATTTAACTGAATATTTTGATATTGCTTGTATCAGGAATTTCTTCA
CTAAAAGAGCATATATGCAACAGTGTGACCTCTTTGGGCTCAATACAAAAGTGATTTGTACAGTGATTCTGTGATTTTCCACCATAATAAATTG
AGAAAGGCAGGTCTTGGTAAAACTTTATAGTCATGTATGAATACCCATATTCAGGTCTTCACCTCCAAGAGACATTCATTTTAATTTATGTCGT
TGTTTATCATCAAGGAAGATTTTGAACTTTTGAGAATTGGCCTTTATCTGTCCTAAACCTCTAACATTCCACTGTATCTTAATGAAACGGAGGT
TCTGTTCTTTCTGCCCAGGGCTTCTGATAAGTGTTTGCAGACTATGTAAACTTCTGAATAATAACTTTCAGTAAGGCAGGAGTCTTAAACCTCT
TAGTATCATTTTGTGCAGTATTATTCTGCTCTGTAAGATAATGTGTTATCAATACTTCATAAGATAACTTTGGCCAGAGAATGAATAAAATTTA
TAGTCACTTTCAAACTGCTCCCTTTTTTTAGGGTGTGGGTCATAGATCATGCAGCTGTCTGCCAACAGATAATCTTGATGAGTAAAAAGAAGCT
AACAGACAAAAAGGTGGCTTAGTCTAAAACACTTAGGAAGGACTTAAAAAATTATTTCCCAAAGAATTAGGGAGTAAAAAAATTGTAAGTACAT
TTGTTTACAGTAAAACAGTGGTCTCTTTGTTTGAGGAATTTTCCTTGCCACCTCAGAAAGTGAAAATTTACTCCTTTTTTTTTTTTTTTTTTAG
AAAATTAATGCTGGTGTGATTGTTTAACTTGTGTAACCTGTTCCTTGAGGTAAAATGAGGAAGAAGCATATGATTTGGCCTCTCTTTTCAGTGA
AGGAAGGAAGTCCATGGAAATTACTGAACCAAACTAGAGATCTAAAGCAAAAGCCCTTTTGTGACACAGATGAACTCAATAGTGCACTGTGACT
ATCTGACTTTTGCCACGTCCAGGCTTAGGCCGCCAAGCGGCCAGGGTCAGCAAATTCCACTACCTGAAAATGACAGAAGGCGGCAGGGAAGGGG
AATTGAGGCATGGGGAGAAGCAGTTGAAGCATACTAGGGAGGCATGAGGTTCTCTCTCTCTCTCTCTCTCTCTCTCTCTCTCTCTCTCTCTCTC
GTGCAGCTAAAGGAAGAGAAGGAAGGAAAAGAGGCTGATCATTGAACGACTTTAGGGGCAAGTCTGGAGTTAGGCGCTGTGGTGTCCAAGCAGA
GCCGTGTCTGGTGGCGGCAAACTAGATTGCTCCAAGCATGGAGCGCCCTGCTTGAGGACACTTGTTGTTGGACTGTTTGTTTACATGCAGTTTG
GGTGGCAAAAAAAGGGGAAAGTCTGGGTGACATCAGCTGCAAGAAGACTTAACCATATGCCGATGTCATCCAACCCCCAATTATTTGAACATTG
TGAAGGTTAAGACATCAATCCATCCATTTTTCTTTCTCTTACTCTGAATCGTGACATTATTTTCTCTAGTTCATTGACCTTGTTGGCAATTTAC
ACCACCAATATAAGCCTCAGGATCCTTTTGTAAAATGAGACTTGACACTAAATTAGTCAACTAAAAATTTAAAGGGTAAAGGGTCCCCCCAGCC
ACCCACTCCTGCCCCTCCCTGTACTAAAGTAGAAAATTTGGACAAGGCACACCATGCGTGTGACTCAATTGTTCTGGCCAAAAAATGGGATGAA
TAATTTTATTTCATAGATGCTTGGTGATTACTGGACTTTACAAAAGTTAACCACTAGTTAAAAAAAAAAAAAAAAAAAAAAAAAAATATATATA
TATATATATATATATATATATACTGCTTTTGGGTACTGAGGTTGAACAATTGGTCGGGGATAATTCCATAAAATGGATTTAAGTATTTAACAAA
TAATGTAAATGTATAGAGCAGGCCTAAAATTTCTCCACTGCCTTTGAGTATACCTTTACAAGTTTACAAATACATCAGCATAAAGCAGTTTCAA
TAAAATGATCAATTGTACAAAGTATAGAAATATACCAAGCGTCTCTTAAGTAAATGATTACAACATTTAGAAGTATTCATGAGTTTGACATATG
CAAAGCAGCCTATCTCTCATGAGTCTGAGTTCCAGCCTCCCTGAACCTTTGTGCATTTCATAGGCTGGTGAACAGCCCAAACATGTAAATAAAT
AGCCATTCAAAGTGCAAGCATTTTTAAAAGTTTAAAACTCCACACAAGTTCTTTTACAAAAGAGTCCACCCTTTTATGTACCTTTAAAAACATT
TAGCCACTTTATTCTTTTGTGTCTTTGAAGAGAAAAAAAATTAATAAAGTTGTAGGATCCTTTTGAATTTAGAAAATGTTGGCAATGTTGGAAA
GAAAGACATTCAGCATTTCAAAGTTCTGGTGGGGAAGGAAAAAAAAGATTCTGAATAAAACAATTTTATGTGATTTTGCAGGTGGTTTGAGCTA
TCATTTTATATGTACGTTTGTACCACTGCTCATTGGAATGTTCATATAATACTGACCTAATTATGCATAGAAAAAGATAAGCCATTTACTTGAT
TCATTTCTTCTTTTTAAATCAAGAAAGGAACAAAAAGGCTCCTATTTTCTGAAAGAAATTGTTCGCTTTTATTGCAGAAAAGTAGCAGCTTGTG
CAGAGCTAGAACTGTAACAATTTTATATTCCAAAGGTTTTGTGTGTATTCGGTTAGTTACAGTAATTATACCCAATAACTGACACCCAAAATCA
CGTAGGAGAAAGCAAAGGCTGTTTTCTCACACAGGCCCTGCTGCAGAACATCTGCTGAGAGATTTGCATATTAATTAACCCATATTAACTCTAA
TTATTCTGGGAAATTATCAAATAAATTGAATTTTCTAATTTTAACCTTTCTTTTACTTGACGCGTGTCGAGGCTGAGAGGACGCACCAGGCCGT
CTCACCTGGGCAATGAGCAAAAGATCTTTTCTACCCCTTTTGCTCCTTTAGACCTGGGGCAGCCTGGGGAGCTTTTGTGTGCCTTGTGCTTTTT
TGGCTTCTGTGCCTGGTAGGGTCTCCATCCATTTGTTGAGAGAGGGAACATGTGTGCTTGTGAGCACAGATGGTGGATTTATACAGGGGTGAGG
AAAGGTGGTACCTCAGAACAGTTGAGCAAACCTTAAGTTTTTTTTTTTTTTTTTTTTTCTTCCTCCCAGCCTGTTGTTTAGGCCTGAGCAAATA
AATGGGAGTTTAATTCAATTAGAGCCTGGCTGTAGTCAGTGCCTCATGACAGAAATTATTCAATATTTTTCCCCTCACATTGTTTGGTTTACAC
ATCACTGACTCCCTTCCTTTTGTACTCTGTTTAGAGTGGCCATAATGTGTTGAGCTTTTGTGTCTGCCATGTCACTGTCCCTTTCACCGTTGTC
TGGTTTTGTCAATTGTGAGGTGCAATGATGCTGTTTTGAAAACAGGGTCCTAGTCAGGGTGTTGCTATGTGAGTAGGGTGGCTGCAGTTTTAAA
CATACTACACAGTAAATAATGTCATTAGCCCAATAGACCTGAATCTACTGCTAAGCCTTAAGAGAAATCCTTTGGGTAGGGAAGTGAACTTTAC
ATGCCTTTCTCAGGTATTAGATGTAGCTTAAAACTGAGATTTCAAAGATAAATCTGGTCAGGGCAGTATGAAGAATTGGACTCCATTGCATGAT
CCCCTTTCAGGCATTTCTTTGGGGTAAGAGGCAAGATTGACTGAGATTAGAATTTTTTGCATGTGATCAAGTTTGTAACGACCCATACTTTAAG
CCACAGCGATCAGGAGAAAGCTACTGATGAAACCTCTACCACAAGCAGATCTCCAGCCCCCCTACTTCGAGCCACATCTCTTCTAAAGACCTAT
GACTCTATTTCCCCCTCAATGATTTCACTTCTCCCCATCCACTAAGATCTATTAAGATGAGCCACACCTCTTGCTACCTTGAAGGTAATTGTTT
TCCTCTGGAAGAGCTGTTCTTTCCCATTATTTCAGCTGATGGAGTAGAAGCATTACGGTACCTCCATGTAACATTCCTTAGACTCAAGAATTAT
TATTGACTTCAGTAAACTCCAGAAAACCAAGAAAGTCACCAAATAGGTCTGTTGGATGGACAAATATTTGAAAGCAATTATTTTTATGGATAGG
CTAGAACTCCAGTCTCCTTTTTCATGGAAGCATGGAATGCCTTTTGCAAGAAAAATATTTCTTTAAGAGACAAGCTGTTGCTACTTCAGATGAA
GGTATGGGTTCATGCTAAGGTTTTTGAGGGGAGGTGGTTTAATTTCGTGGGACTACAATAGCTAAAAGTGTATTTCAGTTATATCTGTATCTTA
GAAAAATGTACAGTTACAGACAAGGTTTAGGGTGGGCCAACTAAAACAATCAGAGAAAATTTTTATAGTTTTTCTATTTCGTGTTTATTTTTTA
CAGCACATTCCAACGTACTCTGGCAGTTACTGACTTTACACCTTTTATGGGCTTTGAAAGTTGTGTAACCTTTTCAGAGGAAGGTGATAGACAC
AATATCTATTTACACAAGGACACATTTCCCCTTTCTTTATATTCCGTTTATTCAAGGTTTTATTTCCAAGGTCTTATTACTATAAAACTTAAAG
CTAGACAAGGTCTGCCCTCATGTCAGATAAAAAAAAATACCAAAACAATTTTGATGGCTCAGAAGCTTATTTTTTCATACTTCCAGGTCCTAAA
TAAATTACAGTTATTTTCACATTCCTCTTGAATATCCTTCAGCCATGATAGTGAGCATATTTTTTCTGTAAAGCCCCTTAATACTTTCCTTCTG
ATGGTTTGTATAGAGCAAGTTGTTGAATGATCGTGGATTCCAATAATTAGAGAGAGTCTTCTAAAATCTTTTTTTCCTTTTGACATATTGTTGG
AAGACATATAGTCACTTGATGTATTTATGAAAGTCTGTGGAATTATTGGCAGTAGATGTACAATATATATGAAATTATTTGTGTATAGCAGTTG
TTTTGGGGGTCTTCCCTCAGCATGCCATCATGCTAGGCTTGCTGATGAAGGTGCTGTTAGACTTTTTAGCCATCAAAGGGCATTATTGACTTGT
GTTCTGGAATATAGCTAACTATTATGTCCTTGGATTGTAAGCATATTATAGATGTTGACAGAAAGTGAAGCTGAAAAAATTTCTAAAGGATAGA
AATAACTACAAAAACTCACTAAATGAAAGGAGCGGTCAGGGGCCTGTATGTTTGAAACATCAATACAATGACTAGTTTCCTTTGGTAGGAGTTA
AGTAAGGTAATCAAAACCATTATGAGTCCTAGATATACAGGAAAACCCGAATATGTTTACTTTTGTAAAAAAATAAACAGATGTCAAGTCTTTG
GGAAATCATAAATTTCACTGAGAACACTGTGACCTAGAAATACCGTGTCTTCTTCAAAGAAAGTGAATTTACTGTTACAAATTCTTCGACCAAT
ATCTGTTTTTAGGATAGCTTGACAAAGGGAGGGTCCTTGCTTTTATTCTTATTTGCCATTATGTTTATATTTTTGATATTAAGGAAGAGGTTTG
GAGGGGAAAATTACATATCTAAGATGTTATTACCTGCATGTTCGACCCTATCCAGAACCTCAACAAATGTATGGAAATTTGAACCTTTTTGAAA
ATCACCAGCATACTGAAATTTGAAAACCTCTCCCAGGTACCAGGGAACCTCCTAATATTTCCAGAGGGAATGAGTGGCTGCCATCTTCCCAAGA
TGCTCCTGTAACCAGGAAGTTGGAGGAGCCTCAGAACTGGCCTCTCTCCACTGCAACCATGGCATTGCAGAAATTCTGCTCTCATTTTATTTAA
TATGCGTATTTTTTTACATAGCTAATCTGAAATGGAAACATTTTTATCCCTATACTGAGAACTGCTTTTGTTTTATCAGCTGACATTTTACAAC
TCATTAATTTATAATGCAGGTTTAATGCCTTCGAATGTTCCTGGGATAAAAAATTAAAGTGGCCAAGCTAGTTTGTATAAAGGCTGACTATAGT
ATGTTAACAATAATTATTTATAACAATATTTTGTATTGGATAGCACATTCCATCTGAATATTTCAAATTGGCATGGTCCCTTCTAACTGGCATT
AATTCATTCATCTTGCATCCCCAAGTAATAAAGTTATTCCCATTTTTAAATGGAGAAATCAAGATTCATGCATTTCAGTGACAGATGTTCAAAT
TCAGAAGAAAATCTCTGTTTGAGTTTGTCTTTGCCTTGTAATACGTATTTGTCACTAGGCCTCCATTATTAAATAAGAAATTGAACCTTGCATT
TAGTCCTATGATCTTTACCCACCCAAGCACGGAGGTAACAAGGTAACTGTGATTTAAAAAAAAAAAAAAAGTCAATGATAAATCCCCCCGGATC
GGCAAGAGTCACTGGACAAAAATTATTGAAAAATAGCTGCATCCTCTCCTCAGTAGCACATACTTGGAAATTGAAATTTTGTGGGGAAGACAAG
GAGGAATAAAGGCTAAGGTTCAGGAGAAAAGTCTTAGAAGGTCTTCCCGACACTTGCCACATGTAAAGACGTTGTAAGAAAGCAGGCCCACAAG
GCCTTTAATCCACTGGCTCAAACATAAATGATCTCTAAGCATGTAAGCATAAGAGATTTAGGAAACCAAATGATTCATAAAATTCCAAAACACT
TCTTTTTTTTTTTTTTTTTTAAAGACAGGGTCTCATTCTGTTGCCCAGGCTGGAGTGCAGTGGTGTGATCATAGCTCACTACAGCCTCGACCTC
CCAAACTCAAACAATCCTCCTACCTCAACCTCCTGAGTGACTGGGACTATTGGCATGCAACACCATACCTGACTGATTTTTTATTTCTTGTAGA
GATGGGGTCTTGCCCAGGCTGATCACAAACTCCTGACCTCAAGAAATTCTCCCTGCTTGGCCTCCCAAAGTGTTCGGATTACAGGTGTGAGCCA
CCATACCTGGCCAAAAGGACTTATTTTAAATTTGAAGTCATATTACCAGCAGAGACATTTTTAAAATGAGGTTACATGTCTGTGTCACGTCTAA
GCTGAAGGATTAGGCATGGTCTCTCTCATCATGGCCTTCCTACTTGACGAAGCATTTCCAAATAAATTTCTAAGTGAAGGGCCAACACAAAAAT
TGTGACTGGCACAAGTTCAAATCTTCCTTTTTCCTCGTAGCCACCACACATTGCAGCACCAGAGAAAAGACAACAGACACTGAAACAATGGTAT
TTCAGTGATTATACCAGTTACATCACCTTGCATTTCCATCTTACTCTTTTATACATCACACAGTGCCATTGCACATACAACTTTGTAGGCTCCT
ATGACAACCTCATAAACAGGGAACCATTATCATGATTTTTACTCATTTCTACCAAGCTTCCTTGGATGATAGGCCAAGGCCACACAGCTGGTTA
CACGTTGGTTCTGGGACAAGGCAAGTCTTCAAGCTCATAATTCTGTGAACTGCCTCTTTATCTCTATTGTTTCCAAACTATTGCAGGAAACATG
GGTGTATTGTTAGATTCTGGTAATTGATCAAATTGGTAGGCTTCTATAACTCAAATTAAGCGGATCCATGAAACTAACAATCATCAAAACAATT
TAAAATTAAGCAACAATCTCCATTTTTGTGGCATGAAAATCCCAAAAAGTCCCAGGGATTTCCATCCCTTACTCTGGTCATGTGGGGTTTAAAC
TTAGATTGCATTCTGCACTGGGAATTTGTATTATCATGTATGTTTTTGTTTGGCTGTTGTACACCTAAAGACATGCTTTCTGGTACCGGCTTTG
CTACCAACTAGCTGTACGGCCTTTGACAAGCCATTTGACCTCACTGTGCCTCTATTACCTCCTTTGTCAAAGAGTTGACTTCAAGTGTCCTTCT
GGAACTACAATTTTGTGATTATATTTCTCCAAATAAATTGTAAGCTTCCTGAGGTGAGAATGGCACTTCCTTCTATCTTCTGGATTTGCCACAT
TGTGGGTATTTAATAAATGTTTGATGATGGCATTGAACGATTTATTGGCACGCTTGAGTAGAGTTATAGGAAATCATTATTGGCTGCCTTATTA
ACAATTAGCTGTCTATAAGCTGCATATGTAATTTTCCTTTACATCAATAGAGTTTCTGCTTCTGGTGAAGACGAAGTATTGCCCCTTCACATCA
GTTCTTCTGTATGTTAATACATTAGCAACTGATATCTTATTTGTACTGTAACTGACAGAATAATGTAGAGAAAGCATATAAGTAGGAAAAGGGG
TTAGTAAGGTAACATTGTATAGGGCATCTTCAAAACAACAGTTCTGCTATTTCAAATGTCAAGCTAATTATCAGCAATGTTTAAATGCTATGAT
AGGCCATTTTTGCAAATGCTGAAATTTTCCAGCAGGTATGGACGTTATAAAAATGTCAAATTTTGCTACTATTCTATTACATGTCACCAGAACA
CAAAAATCTGAAAATAAAAGTTATATGTATAGGCAAGGAAGAGAAACCATTATTTGTTTTGCATATGACTGTAAAATTAATTGGCATAGTGATA
TAATTTAGAGTATTGTTTACTTGGAAGGAAGAGTAGAGAGGATTTAATATA
HUMAN SEQUENCE—mRNA
GGCGATCACGTTTTCACATGATGCTCACGCTCAGGGCGCTTCAATTATCCCTCCCCACAAAGATAGGTGGCGCGTGTTTCAGGGTCTCTCGTCT
CTCTCCTACAGAAAAGAAAAAGAAAAAAATGTCATTAGAAGAGGCGTAACACGTCAGTCCGTCCCCAGGTTTGTGTTTCCTGGAGTGGCCGAAA
GAGATCAGTTCTAACCTGCTCTGCAGGAATAACGGTCCTGCCTCCCGACACTCTTGGCGAGGTTTTTGTACAGTTTGCTCCGGGAGCTGTTTCT
TCGCTTCCACCTTTTTCTCCCCCACACTTCGCGGCTTCTTCATGCTTTTTCTTCTCACCATTTCTGGCCAAAACTACAAACAAGACTTCGCAGA
TCGACCCTGCGTGCTGCCGAAGCAGGGCGCCGAGTCCATGCGAACTGCCATCTGATCCGCTCTTATCAATGAAGCAGCCGATCATGGCGGATGG
CCCCCGGTGCAACAGCCGCAAACAAGCCAATCCCAGGAGGAAAAACGTGGTGAACTATGACAATGTAGTGGACACAGGTTCTGAAACAGATGAG
GAAGACAAGCTTCATATTGCTGAGGATGACGGTATTGCCAACCCTCTGGACCAGGAGACGACTCCAGCTAGTGTGCCCAACCATGAGTCCTCCC
CACACGTGAGCCAAGCTCTGTTGCCAAGAGAGGAAGAGGAAGATGAAATAAGGGAGGGTGGAGTGGAACACCCCTGGCACAACAACGAGATTCT
ACAAGCCTCTGTAGATGGTCCAGAAGAAATGAAGGAAGACTATGACACTATGGGGCCAGAAGCCACGATCCAGACCGCAATTAACAATGGTACA
GTGAAGAATGCAAATTGCACATCAGATTTTGAGGAATACTTTGCCAAAAGAAAACTGGAGGAACGCGATGGTCATGCAGTCAGCATCGAGGAGT
ACCTTCAGCGCAGTGACACAGCCATTATTTACCCAGAAGCCCCTGAGGAGCTGTCTCGCCTTGGCACGCCAGAGGCCAATGGGCAAGAAGAAAA
TGACCTGCCACCTGGAACTCCAGATGCTTTTGCCCAACTGCTGACCTGCCCCTACTGCGACCGGGGCTACAAGCGCTTGACATCACTGAAGGAG
CACATCAAGTACCGCCACGAGAAGAATGAAGAGAACTTTTCCTGCCCTCTCTGTAGCTACACGTTTGCCTACCGCACCCAGCTCGAGCGGCATA
TGGTGACACACAAGCCAGGGACAGATCAGCACCAAATGCTAACCCAAGGAGCAGGTAATCGCAAGTTCAAATGCACAGAGTGTGGCAAGGCCTT
CAAATATAAACACCATCTGAAAGAACACCTGCCAATTCACAGTGGTGAAAAACCTTACGAGTGCCCAAACTGCAAGAAACGTTTCTCCCATTCT
GGTTCCTACAGTTCGCACATCAGCACCAAGAAATGTATTGGTTTAATCTCTGTAAATGGCCGAATGAGAAACAATATCAAGACGGGTTCTTCCC
CTAATTCTGTTTCTTCTTCTCCTACTAATTCAGCCATTACCCAGTTAAGAAACAAGTTGGAGAATGGAAAACCACTTAGTATGTCTGAACAGAC
AGGCTTACTTAAAATTAAAACAGAACCACTAGACTTCAATGACTATAAAGTTCTTATGGCTACACACGGGTTTAGTGGCACTAGTCCCTTTATG
AATGGTGGGCTTGGAGCCACCAGCCCTTTAGGAGTTCATCCATCTGCTCAGAGTCCAATGCAGCACTTAGGTGTAGGGATGGAAGCCCCTTTAC
TTGGGTTTCCCACCATGAATAGTAATTTAAGTGAGGTACAAAAGGTTCTACAGATTGTGGACAATACTGTTTCCAGGCAAAAAATGGACTGCAA
GGCTGAAGAAATTTCAAAGTTGAAAGGTTATCACATGAAGGATCCATGCTCTCAACCTGAGGAACAAGGAGTTACTTCTCCTAATATTCCGCCT
GTCGGTCTTCCGGTAGTGAGTCATAATGGTGCCACTAAAAGTATTATTGACTATACGTTGGAAAAAGTCAATGAAGCCAAAGCTTGCCTCCAGA
GCTTGACTACTGACTCAAGCACACACATCAGTAATATAAAGAAAGAGAAGCTACGTACTTTAATAGATTTGGTCACTGATGACAAAATGATTGA
GAACCACAACATATCCACTCCATTTTCATGCCAGTTCTGTAAAGAAAGTTTTCCTGGCCCCATCCCTTTGCATCAGCATGAACGTTACCTTTGT
AAGATGAATGAAGAGATCAAGGCGGTCCTGCAGCCTCATGAAAACATAGTCCCCAACAAAGCCGGAGTTTTTGTTGATAATAAAGCCCTCCTCT
TGTCATCTGTACTTTCTGAGAAAGGAATGACAAGCCCCATCAACCCATACAAGGACCACATGTCTGTACTCAAAGCATACTATGCTATGAACAT
GGAGCCCAACTCCGATGAACTGCTGAAAATTTCCATTGCTGTGGGCCTTCCTCAGGAATTTGTGAAGGAATGGTTTGAACAACGAAAAGTCTAC
CAGTACTCAAATTCCAGGTCCCCATCCCTGGAAAGAAGCTCCAAGCCGTTAGCTCCCAACAGTAACCCTCCCACAAAAGACTCTTTATTACCCA
GGTCTCCTCTAAAACCTATGGACTCCATAACATCACCATCTATAGCAGAACTCCACAACAGTGTTACGAATTGTGATCCTCCTCTCAGGCTAAC
AAAACCTTCCCATTTTACCAATATTAAACCAGTTGAAAAATTGGACCACTCCAGGAGTAATACTCCTTCTCCCTTAAATCTTTCCTCCACATCT
TCTAAAAACTCCCACAGTACTTCATACACTCCAAACACCTTCTCTTCTGAGGAGCTCCAGGCTGAGCCTTTAGACTTGTCATTACCAAAACAAA
TGAAAGAACCCAAAAGTATTATAGCCACAAAGAACAAAACAAAAGCTAGTACCATCAGTTTAGATCATAACAGTGTTTCTTCCTCATCTGAAAA
CTCAGATGAGCCTCTGAACTTGACTTTTATCAAGAAGGAATTTTCAAATTCAAATAATCTGGACAACAAAAGCACTAACCCAGTGTTCAGCATG
AACCCATTTAGTGCCAAACCTTTATACACAGCTCTTCCACCTCAAAGCGCATTTCCCCCTGCTACTTTCATGCCACCAGTCCAGACCAGTATTC
CTGGGCTACGACCATACCCAGGACTGGATCAGATGAGCTTCCTACCACATATGGCCTACACCTACCCAACTGGAGCAGCTACTTTTGCTGATAT
GCAGCAAAGGACAAAGTACCAGCGGAAACAAGGATTTCAGGGAGAATTGCTTGATGGAGCACAAGACTACATGTCAGGCCTAGATGATATGACA
GACTCCGACTCCTGTCTCTCTCGCAAAAAGATCAAGAAGACAGAGAGTGGCATGTATGCATGTGACTTATGTGACAAGACATTCCAGAAAAGCA
GTTCCCTTCTGCGACATAAATACGAACACACAGGAAAAAGACCACATCAGTGTCAGATTTGTAAGAAAGCGTTTAAACACAAGCACCACCTTAT
CGAGCACTCAAGGCTTCACTCGGGCGAGAAGCCCTATCAGTGTGATAAATGTGGCAAGCGCTTCTCACACTCGGGCTCGTACTCGCAGCACATG
AATCACAGGTATTCCTACTGCAAGCGGGAGGCGGAGGAGCGGGAAGCGGCGGAGCGCGAGGCGCGCGAGAAAGGGCACTTGGAACCCACCGAGC
TGCTGATGAACCGGGCTTACTTGCAGAGCATTACCCCTCAGGGGTACTCTGACTCGGAGGAGAGGGAGAGTATGCCGAGGGATGGCGAGAGCGA
GAAGGAGCACGAGAAAGAAGGCGAGGATGGCTACGGGAAGCTGGGCAGACAGGATGGCGACGAGGAGTTCGAGGAGGAAGAGGAAGAAAGTGAA
AATAAAAGTATGGATACGGATCCCGAAACGATACGAGATGAAGAAGAGACTGGAGATCACTCCATGGACGATAGTTCAAAGGATGGGAAAATGG
AAACCAAATCAGACCACGAGGAAGACAATATGGAAGATGGCATGTAATAAACTACTGCATTTTAAGCTTCCTATTTTTTTTTCCAGTAGTATTG
TTACCTGCTTGAAAACACTGCTGTGTTAAGCTGTTCATGCACGTGCCTGACGCTTCCAGGAAGCTGTAGAGAGGGACAGAAAAGGCGGTTCAGC
CAAGACAGATGTAGACGGAGTTGGAGCTGGGTATTGTTAAAAACTGCATTATGCAAAAATTTTGTACAGTGTTAAGGCCTAAAAACTGTGTGGT
TCAGAGACTAATTCCTGTGTTTAATAGCATTTATACTTTAAGCACAACTAGAAAATTGTAAGAATTGCACTCTACTTATGTATCACTACAAACT
TTAAAAAACTATGTCTAATTTATATTAATACATTTTAAAAAGGTGCCCGCACTACCATACATCAGTATTTTTATTATTATTATTGTTATTCCTT
TTTAATTTAATGTGCTCGCACTACAATGCATCAGTATTATGATTCCTCTGTACTTTCCTTTCGCTATTCATCAATTTCCCATTTTTTTTTTTCA
GCTTAAGTAACCACACAATTTTAGGCCTCAATTTTTTTTTTTTTCTGTGAAGGAACTTGAAGTGATGCATGTGTGAATTTAAGATACCGAAGTC
TTAAAGTGACCTGGACGTGAAGGAAAAAGTAAGATGAGAAATAAAGAAAGCCTTTGTAAGGTGGTTTTAAAAGCCTTATATGCAAACCTTTTAA
TCTGTGTTTCTGCAAGTCCCATCCTTGTACAGTGTTAAGAGGGTAACATGGGTTACCTTTCCACCAGCTTCAGTGTTAAGCTCACCCTGTTCTT
TGAAGCACCCATGTCAGTATTAGAAGAATAGGCAGCAGTTCCTTAGTTTACATATGTTTGTGCAATTATTTTCTGTACTTTTTTGTTCATTAAT
TTTGTCAGTATTACACCAAACTGTTTTTGCAACAAAAAAATTTTTTTTGCATTCATTTAATTTTAGGTCAAATAACATTTTATTTATGTGGCTC
ATTTTATATTTCCTAATTTTATTTATTTCATACTGTAGTGTACAGTATTATAGTTCTTCAATATATACATATATTTTAGTAAAAAAGGAACATG
ACGTTGATCATTTGGGCAAATTTTACGTAAAGAGAAGAGCATTTATTGTGTTTTGGAACATTAATTGTGAGATGGGATTTTTCAATTTTATTAT
TTTATTTTTGTTTTTTTCCAATTACTGGAAATTCCAAATTTGGGAACTTTTGATACGATCTTGTGAAAACACTGTATTTTCGACTGAAAATTCC
ACTTTCTTCATCTTGTTTTTTAGCTAAAAAGAGGGACTGTTAAATACAATGTATGATACCATGACAAAAATCTTTCCTGAATTGTCTTTGTAAA
AGTATTATTGAATTTTCAATTTGTAATTTCTTTTGAAAATGACCATGCTCGAATAAAAATGTAGCCAAACT
HUMAN SEQUENCE—CODING
ATGAAGCAGCCGATCATGGCGGATGGCCCCCGGTGCAAGAGGCGCAAACAAGCCAATCCCCAGGAGGAAAAACGTGGTGAACTATGAAATGTAG
TGGACACAGGTTCTGAAACAGATGAGGAAGACAAGCTTCATATTGCTGAGGATGACGGTATTGCCAACCCTCTGGACAAGGAGACGAGTCCAGC
TAGTGTGCCCAACCATGAGTCCTCCCCACACGTGAGCCAAGCTCTGTTGCCAAGAGAGGAAGAGGAAGATGAAATAAGGGAGGGTGGAGTGGAA
CACCCCTGGCACAACAACGAGATTCTACAAGCCTCTGTAGATGGTCCAGAAGAAATGAAGGAAGACTATGACACTATGGGGCCAGAAGCCACGA
TCCAGACCGCAATTAACAATGGTACAGTGAAGAATGCAAATTGCACATCAGATTTTGAGGAATACTTTGCCAAAAGAAAACTGGAGGAACGCGA
TGGTCATGCAGTCAGCATCGAGGAGTACCTTCAGCGCAGTGACACAGCCATTATTTACCCAGAAGCCCCTGAGGAGCTGTCTCGCCTTGGCACG
CCAGAGGCCAATGGGCAAGAAGAAAATGACCTGCCACCTGGAACTCCAGATGCTTTTGCCCAACTGCTGACCTGCCCCTACTGCGACCGGGGCT
ACAAGCGCTTGACATCACTGAAGGAGCACATCAAGTACCGCCACGAGAAGAATGAAGAGAACTTTTCCTGCCCTCTCTGTAGCTACACGTTTGC
CTACCGCACCCAGCTCGAGCGGCATATGGTGACACACAAGCCAGGGACAGATCAGCACCAAATGCTAACCCAAGGAGCAGGTAATCGCAAGTTC
AAATGCACAGAGTGTGGCAAGGCCTTCAAATATAAACACCATCTGAAAGAACACCTGCGAATTCACAGTGGTGAAAAACCTTACGAGTGCCCAA
ACTGCAAGAAACGTTTCTCCCATTCTGGTTCCTACAGTTCGCACATCAGCAGCAAGAAATGTATTGGTTTAATCTCTGTAAATGGCCGAATGAG
AAACAATATCAAGACGGGTTCTTCCCCTAATTCTGTTTCTTCTTCTCCTACTAATTCAGCCATTACCCAGTTAAGAAACAAGTTGGAGAATGGA
AAACCACTTAGTATGTCTGAACAGACAGGCTTACTTAAAATTAAAACAGAACCACTAGACTTCAATGACTATAAAGTTCTTATGGCTACACACG
GGTTTAGTGGCACTAGTCCCTTTATGAATGGTGGGCTTGGAGCCACCAGCCCTTTAGGAGTTCATCCATCTGCTCAGAGTCCAATGAAGAACTT
AGGTGTAGGGATGGAAGCCCCTTTACTTGGGTTTCCCACCATGAATAGTAATTTAAGTGAGGTACAAAAGGTTCTACAGATTGTGGACAATACT
GTTTCCAGGCAAAAAATGGACTGCAAGGCTGAAGAAATTTCAAAGTTGAAAGGTTATCACATGAAGGATCCATGCTCTCAACCTGAGGAACAAG
GAGTTACTTCTCCTAATATTCCGCCTGTCGGTCTTCCGGTAGTGAGTCATAATGGTGCCACTAAAAGTATTATTGACTATACGTTGGAAAAAGT
CAATGAAGCCAAAGCTTGCCTCCAGAGCTTGACTACTGACTCAAGGAGACAGATCAGTAATATAAAGAAAGAGAAGCTACGTACTTTAATAGAT
TTGGTCACTGATGACAAAATGATTGAGAACCACAACATATCCACTCCATTTTCATGCCAGTTCTGTAAAGAAAGTTTTCCTGGCCCCATCCCTT
TGCATCAGCATGAACGTTACCTTTGTAAGATGAATGAAGAGATCAAGGCGGTCCTGCAGCCTCATGAAACATAGTCCCCAACAAAGCCGGAAGT
TTTTGTTGATAATAAAGCCCTCCTCTTGTCATCTGTACTTTCTGAGAAAGGAATGACAAGCCCCATCAACCCATACAAGGACCACATGTCTGTA
CTCAAAGCATACTATGCTATGAACATGGAGCCCAACTCCGATGAACTGCTGAAAATTTCCATTGCTGTGGGCCTTCCTAAGGAATTTGTGAAGG
AATGGTTTGAACAACGAAAAGTCTACCAGTACTCAAATTCCAGGTCCCCATCCCTGGAAAGAAGCTCCAAGCCGTTAGCTCCCAACAGTAACCC
TCCCACAAAAGACTCTTTATTACCCAGGTCTCCTGTAAAACCTATGGACTCCATAACATCACCATCTATAGCAGAACTCCACAACAGTGTTACG
AATTGTGATCCTCCTCTCAGGCTAACAAAACCTTCCCATTTTACCAATATTAAACCAGTTGAAAAATTGGACCACTCCAGGAGTAATACTCCTT
CTCCCTTAAATCTTTCCTCCACATCTTCTAAAAACTCCCACAGTAGTTCATACACTCCAAACAGCTTCTCTTCTGAGGAGCTCCAGGCTGAGCC
TTTAGACTTGTCATTACCAAAACAAATGAAAGAACCCAAAAGTATTATAGCCACAAAGAACAAAACAAAAGCTAGTAGCATCAGTTTAGATCAT
AACAGTGTTTCTTCCTCATCTGAAAACTCAGATGAGCCTCTGAACTTGACTTTTATCAAGAAGGAATTTTCAAATTCAAATAATCTGGACAACA
AAAGCACTAACCCAGTGTTCAGCATGAACCCATTTAGTGCCAAACCTTTATACACAGCTCTTCCACCTCAAAGCGCATTTCCCCCTGCTACTTT
CATGCCACCAGTCCAGACCAGTATTCCTGGGCTACGACCATACCCAGGACTGGATCAGATGAGCTTCCTACCACATATGGCCTACACCTACCCA
ACTGGAGCAGCTACTTTTGCTGATATGCAGCAAAGGAGAAAGTACCAGCGGAAACAAGGATTTCAGGGAGAATTGCTTGATGGAGCACAAGACT
ACATGTCAGGCCTAGATGATATGACAGACTCCGACTCCTGTCTGTCTCGCAAAAAGATCAAGAAGACAGAGAGTGGCATGTATGCATGTGACTT
ATGTGACAAGACATTCCAGAAAAGCAGTTCCCTTCTGCGACATAAATACGAACACACAGGAAAAAGACCACATCAGTGTCAGATTTGTAAGAAA
GCGTTTAAACACAAGCACCACCTTATCGAGCACTCAAGGCTTCACTCGGGCGAGAAGCCCTATCAGTGTGATAAATGTGGCAAGCGCTTCTCAC
ACTCGGGCTCGTACTCGCAGCACATGAATCACAGGTATTCCTACTGCAAGCGGGAGGCGGAGGAGCGGGAAGCGGCGGAGCGCGAGGCGCGCGA
GAAAGGGCACTTGGAACCCACCGAGCTGCTGATGAACCGGGCTTACTTGCAGACCATTACCCCTCAGGGGTACTCTGACTCGGAGGAGAGGGAG
AGTATGCCGAGGGATGGCGAGAGCGAGAAGGAGCACGAGAAAGAAGGCGAGGATGGCTACGGGAAGCTGGGCAGACAGGATGCCGACGAGGAGT
TCGAGGAGGAAGAGGAAGAAAGTGAAAATAAAAGTATGGATACGGATCCCGAAACGATACGAGATGAAGAAGAGACTGGAGATCACTCCATGGA
CGATAGTTCGGAGGATGGGAAAATGGAAACCAAATCAGACCACGAGGAAGACAATATGGAAGATGGCATGTAA

TABLE 37
MOUSE NOMENCLATURE
ICSCNN Vdac1
Celera mCG3636
HUMAN NOMENCLATURE
HGNC VDAC1
Celera hCG16588
MOUSE SEQUENCE—GENOMIC
TTTGATCCTGGAAAGGCATCAGAGGCTGGTGTCTTTGGTAAGAAGGGCTGTGTACCCTGAACCTGGGATATGGAGGGGTCTGATGGGTTTGGGG
GAAGAAGCTGAAGAAGTTATGGCATGTCAGAGCAAGCCCTTAAGGTCAGTTTCCTGCCTCCCTCCTCCAGGGTACTCAGAATCCAACCAGCTTG
TTGATTTATTATCTTTCTAACTGTCACTGTCAAGAAAGTCCTCTCCCCGAGAGGGACAAAGGGCATTGTAGGAAGGGCAAAAAGGATCCCATAG
TTGGAGATGCCTGAGCGGATCCAGACTTTGCAGGTAACATTGTGTGTATGTGTGTGTGTGTGTGTGTGTAATTTCCACGGAGGTGTAGCAGTAG
CAAGCCGTATCAGAGGCAGTACAGACACTACTGTCAAGATAGGCAACAGGTCACATCCATACCCTCTCTCCTGACACCAATTACAAACCTAGGG
ATCCCTGAGGCCACCCTCACATTTAAGAACTCACTGGGATCTCTGTCACTCCCAGTCACAGGTAAAAGCAACTCAGGAAAGGGTCAAGTGGGCA
AGACCAGGAGGCGTCCCAGGGAACTGTCTGCCGTCTCCTGCAGATGGAGTCCCAGCTGGTGCTCACTGCTCTCCAAATGTGCCAACTCACACGG
AGCACCATCCGCTGCCTCAGGTGCTACCTAAAGCGCATATTGGGGTTCCATACTTGCTGCCTGTGAGGTCAACACCAGTCCTTCAGATAGTCTA
AGGCCCAGCTTCGTAAGCTTGGCTCAAAGCTGGTAACCATGGCAATCTCGTTGATTTACACTGTAAGGCCAACTCTCTAAATGAAGATCCGCTC
CTAGAACAGCACTTTGTGGAAGCTAAGGGCAAAGGCCAGACCTCCTTGTAGACAAGGTTAAGAACTTTACTTGAATGTAGATAAATTCAACTTC
TGGCCTGCATTAGGCTGCAGAGGCCATTCTTCAGGACTGAAACCCCCCAGGACATGGTCCTCAGCTGCACAGACTGTAGAAGGAAGCCCAGGGG
ACTGGCTGCCTGCTTGCATGCTGTAGTCACCTGTCATACCCTGATGGCATGTTGAACACCAGCTGGGGGACAGCGTCCTGGAAAGGGACAGGGT
GGACAAGCCAAGGCCAGGCCTGACCCCAGGGCAGCATTGATTCCAGAAAGGAAGTTAGCAAAGTTGCCATTAGGTATTGAGCTCTATGATGGAA
GCAAAAAGCCAAGTTCAAAACAATGTGGAAGGAAGAACCTACCTTTGAATAGGAGTAGAGGAAGTGACCACCTGGTGGAGGATGTGGTTCCGTT
GGCAGAGTGCTTAGTGTCTAGCATGCAGGAAGCCCACAGCCCTGGGAGATAGAGGTAGGAAGATCAGAAGTTCAAACTCATCCTTGGCTACCTA
ATAGTGAGTTTGAGGGAAGCCCGGGCTACGTGAGATGCAGAGATGCAGTGTCAAAACAAAAAACAAAACAAAACAAAACAACAACAACAACAAC
AACAACAACAAAAACCCAAAAACAACCTTTAGTCCTAGCACCCAGGAAGCAGATATAGATGGGTCTCTTTGGGTTCAAGGCCAGCCTGGTCTAC
ACTGTGAATTCTATAACAGCTAGGGCTACATAGTGAGACTATGTCTAAAAACAACAAAATTCCCCCAAAAAACAAGAATCAAGTAATGCTTGTT
CAAGTCTTCTTTGTTTTTGTTTGTTTGCTTGTTTGTTTTTTGTTTTGTTTTGTTTTTGTTTTTCAAGACAGGGTTTCTCTGTGTAGCCCTGGCT
GTCCTGGAACTCACTTTGTAGACCAGGCTGGCCTCGAACTCAGAAATCTGCCTGCCTCTGCCTCCCAAGTGTTGGGAGTGCACCACCATGCCCA
GCACTTGTTCAAGTCTTAAGGAGTTGGGTTGTTAAGTCAAAGGACAGAGGCAGGTACAGCTTTGCCCCTAGGGCAAACCCCATGCATCTGTTCA
CTTGTGTAGCCTGAGGAGACAATGCTATCTCTTGCAGTAGTATGGTATCTTAGCCAATGATCGATTGATCGATCTCTCTCTCTCTCAAATACAC
ACACACACACACACACACACACACACACACACACACACACACACATATGCGTGCTGAGCAGAGAAACTCTGGCTTCTTTCCCACTTAAGGTCTC
CGGTAGGAAGGCAGCCTCTGACAATTTCACTTTCCACACTCCAGCCCAAATTGGAGAGCCTCACAGCTGCCAGGGCAGGCAGATAAGTTTGCCA
CCCAATAGCAGAAACGACCGCACTTACTCCAAACCCTAATCATTCCAGTGCAGCAAATGGAGAAAGAGCCTCTCTCTTTATTAAGATTTTTTTT
AACTTATTTTTATTTTTTGAATGTAGCCCTGGCTGTCCTGGAACTCACTATGTAGTCCAGGCTGGCCTCAAACTCACAGAGATCCTCCTGCCTC
TGCCTCCTGAGTGCTGGGAATATAGGCGTGCACCACCACGCCCAGCTAAGAAGAGATTCTAAAATACAAGTGTGTGTCTGTGTGTCTATGCCCA
TGAGTGTAGTGCCTGCTGAATCTGAAGACTGAAGCTCGAGATACAGGGACATGGGTCACGGGTGGTGGAATGCTGACGAGGGTCTTCTAGAAGA
GCAGAAAAGGGGGCTGGTGAGATGGCTCAGTGGGTTAGAGCACCCGACTGCTCTTCCGAAGGTCCAGAGTTCAAATCCCAGCAACCACATGGTG
GCTCACAACCATCCGTAACAAGATCTGACCCCCTCTTCTGGAGTGTCTGAAGACAGCTACAATGTACTTACATATAATAAATAAATAAATAAAT
AAATAAATAAATAAATAAATAAATAAATAAAAAAGAAAAAAAAAAAAAAAAGAAGAGCAGAAAAGGTACTTAACTGGTACCTTCAGCCTCCTGT
CCCGGATTTGAGACAGGGTCTCACTCTGTAGCACAGGCTGGCCCACATCCATAGAGGCAGGGAGAGCAATGGGGTTCCAGGGGCAGAGGAAAAG
GCACAGGGAGGCATCTGATGATACAAAGTTTCAGCTACACATGTTACATAATTTCCAGAGCTGTACCTGCTGAAGCGTCCATGGCTAACAGTCC
CGGGACAGGGATGGCTGCTGTCCTGTCTATTTGATGGAGTTTACAATTTGTAAACCTGGGCATGCCGGTGAGGGAGTTTCTAGAGTGGGTTGAT
TAAGGAGGGAAGCCCCGTCCTAATGCTGGGTGGCATCTGCTCAGGGGCTTAGCCTACATAAAAAGGAGAAAGTGGGCTGAGCCACCAACATTCA
TCTCTCCCTTCTTGCTGACTGCGAATGCCCTGTGACCCACTGCATCATGTGGCTCTTGTCATGTCTTTCCTGTTATGATGGCCTGCCTTCTCAA
CTCCCAAGCCAAAATAACCCTGTCCTTCCTTGAGTTGCTTTCGTCAGACACTGTGTTTCAGTAAGAACAGTCACTCATACAATGGTGGATTGTA
AGTTTAAGTTAGATGTGAAGACAGATTTTTTTTTCCCTTTGGTTTTGTTTTGTTTTGTTGGTTTTGAAAGACAAAGTTTCTGTGTGTAGCCCTG
GAACTTACTCTGTAGACCCGGCTGGCCTGGAACTCAGAGTTCTGCCTGCTTCTGCCTCCTGAGTGCTGGGATTAAAGGAGTATGCCACCACTGC
CCTGCTTGCTTTGGTTTTCTGAGACAGGGCCTTGCTCTGCAGCTCTAGGCTGGTTTTAAACATAAAATCTTGTAGCTTCCCTCATGCACACACA
CACTGCCACTAATATAACTAATTATAATTATATAATAATTTTTTAAAGTTTCTGAAAGCAGATGGTAGACAAGATGGCTCAGAAGTTAAAAGCA
TTTGCTGCTCTTATAGAGGACTATGGTTCAGTTCCCAGCACTCACGGGGAGGCACACAATGATCCTTAACTCGTTTCAGGGGATCTCGCGCCCT
CTTCTGGCCTCCATGGACAATGCATGCACATGGTACACAATCATCTATGCAGGCAAAACACTTATCACATAAAATAAAAATAACATATAAATCT
CCTTTTAAAAAGTCAGGAGAGGGGCTGGAGAGATGGCTCAGAGGTTAAGAGCACCGACTGCTCTTTCAGAGGTCCTGAGTTCAATTCCCAGCAG
CCACATGGTGACTTAAAACCATCTGTAATGGGATCCGATGCCCTCTTCTGGTGTGTCTTTAGACAGCTACAGTGTGCTCATATACATTAAACAA
ACAAACAAATAAACAAACAAATAAATCTTTAAAAAAAAGAAAGCCAGGAGAGATTGGTTAGTGGGTAGAGATGCTTACTATGGGGGCCTAACTA
GTCTTTGGAACCTACAGAGGGAAACTGACTCCATACGATCTTCTCCAACTGTAGCCCCCTCCCCTAATATTAATTAAAGCAATTGTTTTTCAAA
AGATTTCTTTAGTTTTATGTTATGTGTATGAGTGCTTGCCTGCATGTATGTAGATGTACCACATACTTGCCTGGTACCCTCAGAGGCCAGAAGA
GTGTTAGATTACCTGGAGCTGAAATTACAGGTGTTTATGAGCCAATATATGGGTGCTGGGTCCTCTGCAAGAGCGGCCAGGGTTCTTAATGGCT
GAGCAATCTTTCTTTCTTGTCCCTAAGATCAGTCATTACAAGGGAATGTAGGGATGTGGTTGGCATCTGTACAGCTTGTTGATGGAGATAGATG
GTTTCCTGGGTATTTGTATCTAATGTCATGGAAATCTATGTATTAAACATGTATAGTTTTTTTTATATGCCAACCACACTTCAGGAAAACAATT
TATTCTTATTTTGTATTATTTATGTATATACATGTGTGTGTGTAGGGGTTGTGAATGTGTGCCACATGGGTACAGATACCTACAGAGACCAGAA
GAGGGCGATAGATCCTCTCCAGCTGGATAACAGGTGGGTGTGAGCTGCCTGAAGTGTGTGTGTGTGTGTGTGTGTGTGTGTGTGTATAACTGAA
CTCAGGTCCTCTGGTAGAGCAGGAAGTGCTCTTAACTACTGAGCCATCTTCAGCCCCAAGAAAATAGATTAAAAAAAAAAACTCTTTAATCCCA
GCATTTGGGAGGCAGAGGCAGGAGAATTTCTGAGTTCGAGGACAGCCTGGTCTACAGAGTGAGTTCCAGGACAGCCAGGGCTACACAGAGAAGC
CCTGTCTCAGAAAACCAAAAACTAATCAAACCAAACCAAAACCAAAACAAAAAAAACCCTGCTAGCCTCGAATCCTAGGACTCAGGAGGTAGAG
GCAGGAGGATTTCTATGAGTTGAGGCCAGCTTGGTCTGTCAATATAGTGAGTTCCAGGCAACCAGGAGTATATAGAGAAACCCTGTCTCAAGAA
ACCAAAGTGGGTGGAGATATACCTTTGTGGTTAAGAGCACTGATTGCTCTTGCCAAGGACCTGGGTTCAATCCCTAACATCCATATGTTGGTTC
ATAATTTTTAACACATGCATGATAGTTCACAACCATCCAGAGCTCTAAGGGATCCAACGCCTTCTTCTGACCTGCAGGGGCATCAGGCATGCAT
GTTGCACACAGACATATAAAGGCAACATCCTTAGTGTAAAAGAAGTAAGTCTTTTTAAAAAAATAATCTTTGCTGGGGATGATGGTACATACCC
ATAATCTTAACAGTTAGGAGGTTGAGGCAGGAGGATCACGAGGAGTTAGTCAACTTCAGCAACATACCAAGACTATGCCTTAGAGATTGAATAT
ACTCACAGGGCCCCAAGTATAGGATGGGCATCGCTCGCCCTTGTTGGGCCTGCTATAACTTGCCAGCAGTCAGTACTGTGGTTACAATAATGTG
GATAGCGTTATAATTGCTCCCATTAATATTATGAATGAAGATTTCCTCAGAGGAAGCCTCTGAGACACTTTAGCAGGCTGAAGATGATGGGGGT
GGGTCAGGACCTAGGGCCATTTTATTCCCTCAATCCCCAGGACTCACATGGAAAAGGGAAAGAACGGATCCCTGCAAGCTGTTTTTTAACCTGT
GCATGAAAGTATAACGTAAAAACAAAATTCTAGGCCTTTAGGCCCAGGCTTGGGAATGTGACCTCTGTGACTTAATGCTCCAAGGTATGCTAAT
CATAAAACAGATAGCGACATTCCTCCACCCGCCAGCTTCCTGGGTTCAAAGAGTGTTCATTCACAGAAAGTCACCCTGACCATTGCTGGAACCC
GCTATGCAAATGTGCTATTGTTAGGGGCTCTTAGAAACTGTCTTGAGAGATAACAGGAATAATTACCAGGTATTCCTTGTGACTCTTGTGACTT
TGTACTTCCTTGGGACTCTTAACTGGTATCTTTGGTATTTTCCAACAAAGCCCTCCCCACTTCCTTGAGTTGTGGTTTCTTCCTTTAAATACCC
CCTTACCCAGCTACTTGGGGTGCCACGGTCCTCTACCCCTGCGTGGTGTATGACCGTGGGCCCGAGAGAGCGCTCTTGAATGAGTCCTCTTGCA
GTTTGCAGCAAGACCGTTTCTTGTGTGATTTGGGGTGTCGCCTCTCCTGAGTCAGAACGTGGGGGAGTCCTCACGTTGTGGGTCTTTCATGCAC
ACATAAACAAAAACTTAAAATATAATTTTTTAAAAGAAGACCTAGTATAAGAAAGAACTTCCCTAAGGTGAAGAAAAAAAATACTTTTTTTTTT
TTTTTTTTGAGACAGGGTTTCATGTAACCTAGGCTGGGTTTGAGGTCATTATGTAGCCAAGGATACAGTAAACTTCTGATCCAGCCTCTGAAAT
AAAATAGTCTCACACGCATAGACCATTTCATTATTAAGAGAAAACTGCATCCCCTCTGGATGGTTTACTTTGTTCTGTGCAAAGGAAGACTGCA
ACTGATTTTGCACCTAAAAGAAAATACAATGTAAATAAATTAGGCAAAAAGAAAGAAAGAAAGAAAGAAAGAAAGAAAGAAAGAAAGAAAGAAG
GAAAGAAAGAAAGGAAGAAAGAAAGAAAGAAAGAAAGAAAGAAAGAAAGAAGGAAAGCAAGCAAGCAAGCAACCCATTACTCAGGTAGCTGGCA
AACAGCAGGAGGGCCAGATGCAGGTGCACACTAGCTGCACAGCTAATCTTAACCCTAGCGTCAGTTTTAGTCCTATTTTCACAGCTAGAGAACT
CCAGGGTCTCAGTGATTCCTCCCCAGAGTCAGGGGAAAGAGAGCAGGAGGACTCTGTCACTTGTCAGCTCCAACTCCAACGAAGCCTGTGCATG
AGACAAAAGTGAAATAGGGTCACTTATCCCAACAGACCTATGGTCAGGACAAAAGTGACATAGGGTCACTTACCCCCAACAGGACTATGGTCAG
GACTGGCCCATCCCCACAAGTCAACAAGACATCTCTGGACTCACATGGAACTGAAGTAGCTCTGGGGACCCCAAACTCAGGAGGGGGATCCATG
AGGAAATGAGCTCAAAGCGTCTTGCAACTGCACAGTGTTCTGCTTGCTTTTCTGGGCCAACAGAGATGCAAGCCTGCCGGAGGAGTTGGCAGAC
TCAGGACATCCTGCTGTTTCCCTAAAGCTTCTGGATTTCTTAATTTGAATCTGCAAGTAAGAACCCGACTGGCCAGGCTTGGATCAGGTTTTCT
GTCTCAGGCCAATCAGCTTTGGCAGACTCCGTAGAGGGGAGGCTGGGCAGAGTCCAGTTTAAAAAAAAAAAAAAAAAGGTGACTTGCTTTTCCC
CTCCCTGCAGGCTGTGACTGGGAAGAAAGATCAATGAATAACATTTCTGGTGCACTTGGATTTCAATCCCAGGGGCTCCCAGGTCTCCTCCCTA
TTCTGGCTGATTTTCTTTGCCTTTCTTGTGTGGCTAATTCTTTGGATTTAAAACCTTCCCTTCTGGAAGGAAAGAGATTCATAAAAGTCAGGTT
CAGTCCTCAGCACTAGAAAGTTAAAAACAAACAAACAAACAAACAAACAAAACCCAAAACATACATGGCTTGGGAAGTCCAGCTATCGGACTCA
CGAGGTCCCTCTAGTCAACAGTTGCTGGAGAGGGAACTCTTGAGCTGCTGATAAGTCATGCATTGGTCCAGGGATGCATGCAGGTTTGTTAGTT
GGGGGCCTTTGGGGTGATCTTTTTAGTTACCTTGAGTTACCTTGTAAATAAATTAAACACTTTCCCTGGTTTGCCGACGTAGACTTTGGGTGGA
ATTCTTTAATCTGTCATCTGTGCCTTCTCTACCGCAAATAGCTGTCTGTCTCCCCAGCAAAAGTGACACAAGCCACTCGCTGAGTTTTAGGTAA
GTGCCTTACCAGTCCTCTGCATCTCCATTCATTTTAAATTAAAACCAAGACTCCCAGGGCGCTGAACGGAGGTGGCGCACACCGCCTTTAATCC
CAGCACTTGGGAGGCAGAGGCAGGCGGATTTCTGAGTTCGAGGCCGGCCTGGTCTACAGTGTTAGTTCCAGGACAGCCAAGGCTATACAGAGAA
ACCCTGTCTCGGGAAAAAACAAACAAACAAACAAACAAAAAACAAAAACAAAAAAAAAACCCAAAAAATAAAACAAAACAAAAAAAAAAACCAA
GAAAGACTCTCAGGGCGAAGTTCCGAGAAGCCAGACTTTCTGCCCACCCACGACACACAGCCCTGGGCATCAGGGCTCTGGGTCAAGTGTTAAG
ATTCTTGGAGACAAATCTTGTTGTCCCAGGTCTTGTCCTTCCGACTCTGCCTCCCTGTACGCCCCTGTGACTCAGCAGCTTTCCCTGCCTGGCT
TTAACTCCGGGAATGGGCTGGGTGTTTGTTGCCGGGTAGATAGAGCTGCAGACTTGGGAACAGGTTCTTTCTAATGCAAAGCAAGCCCTGTACC
ACGGAGCTATCTGTTTGCCCACATTCTATTTCGAAGGGCTTTCCCTCATAGTCTTAACTAGATATGGGCTTAGGCCCCCTTACATTCCCTCTCC
TCGGCCTCTCTCTCTCTTGGTTCCCTTCCTTTAGCCCACGGAACAGCATTTTCAATCTAGAGTGGCTGCGGATGCCCAGCTGTCTTGTTCAGCT
ACAGCAGGTTGTGAGCAACCACCTGGCGTCTCCTCTACTGGACCCCAAATGCCTGAATTCTCCTCGGACGTCTGTTAGTAGAAGGCAAAGGGTG
TGAAGTCTTCACTCACATGTGATTTCACAAACTCTCCAAACCTGATTTGGTTAGTCTTGGTGTGGCACGCTCAACAGGGCAGGAGGCCACGTGC
TGAGACCAGGCTGGGAAAAAGTGTCCGCCCTCTCTACACTGCAGGAAAGCAGACATCCAGAACCCGCGTGACCTCACGTAGCGAGACTCCGCAG
TCTCACAAACCCACTTTGTCTTTTTTAATTTCTTAAATGCTTGGGAAATGGCGGAAAGAGGCTTTGACACTAGGGAAGATTGCACGCAACTTTA
CAGTGCGGAGCCGGCTCTCGGGAGAGTTTAATTTGCAACTGACTCGCTGACGCACGCGGGCGTGTCGCAGGCTCCGGAAATCAAGGTTGGAGCA
GAGGGGACAGTAGCCGCTCTGACCTATACAGCGCCACTTCCAGAGCCCAGTGTCCCCACTGGGCCTCCGAGTGACTTGCAGCCTCGGCGACTCC
GGCGACACTATCCGCTGAGGTCCCTAGGCCACCGCTCTGTGTGCCGGAAGCTCCCCTCCCCCTCTTCCCAAATGCTTCCAGCAGCCTGGAGGCT
GCAACAGTGACCCGCGTGTCCCCGCGCACGCACACACCTCACGCACGTCAGACCCACGCCTCCCTATCGCCCCGTGCCAGAGTGCACGCAGGCC
TTTACGCGCGGCCACACATGATCACAGAGCCGCAGAGGGCGAGAGCGCGGCTCGCGAGTGGGAAGAAGACCCCGAGACTGGTCTGGGCGCTGTC
CGGGGTGGAGCCGAGGGCGGAGCTTCGGGGTCCGGCGCGCAGAGCTGGGGCGTGTCCGCGCCGCCGCGATCTGCGCCGCTCCGCCCTCGGCCCG
TTCGCTGCGCTCCCAGCCTCCTCGCCGCAACCCCCGCCGTAGCTGCCGCTGCTCCCGCCGTCACCGCCTCCGAGGTAGGCGCAGCGGAGAGGGC
CTGGGCTATGCCTGAGTCCCCAGGGCCTCGGGTCGCGCTCCTGGGAAGTCTCCAGGGAGGCGATCGAGGGAGGTTCGGGCGAGGGCGCTGGGCC
TAAGCGCTTGGCCCCGGGAAGGGTGAACCAGGGACGATCATTGCGGGCCTGGAGGGGCAGCGGGCTTGTAGAGGAGGCCTGGAGGGCGGGCGCA
GAGCGGATCTCCCAGATTCTAGCTCGAGCGTTCAGAGCACCTGCATTTGGAAGCCAGGGGCGGTTCGAACATGCGAGGAGCGGAAGGGCAAGGG
CGGCCGCGTGGAGGCCCGGCGGGCCGCGGGTCTAGGTGACCTTCCCGGCGGCGCCCTCTGGCTCTAGATCCCGTCGGTGTGGATCTGGTAGAGA
CCCTCCCACGGCCCAAGCCCTTCCCCACCAGCTCCAAGGGCCTGTGCAGACCCAGCTTCCTCCGCCGAGAGGACGAACTCCAGGCACGGCCATG
CCCTGGATCCCCCAGCCCCTACACGCCCTAGCCGGCCCACGCCCGGAGTAGGTGGCGCCGGGTCCCTCCCGCGCATGCCAGTGTCTTCCCGGGC
GGAACGGGCTGCCCTGAGTGGACTTAGGCCGCAGGAACAGGGACCTGGGCCTGGACTGAGAGCGAGACCCCATAGGGAGCCATACGCTTGGCCT
CCTTGGCCTCGGGGACGGAGTCCGTGTTCTGTGTCGCCAAATGCAACTGTGCATTCTGGAAACTGTTTTCAGGGCCTGACATCAGATTGGAGGC
CCGCAACTCTACACGGTTTGTCCAAGGTCACACTGGTAGGAAGATTTAAAACTTCAATCTTCTCATCTGCGAAATAGGAGCAATTATATACACT
TCACTGAGTTTGGGGGAAAGAATAAAATGATCCGATTAACACGAAGCCTTGGGGCATGCACAAGGACACCTTAGAAACCTTGACATTTGCCTAG
GAGACCTAGGAGCTTTTGGGGAGGGTTGAACTCATCCTCCACAGACTTTTATTTTATTTTATTTTTTATTCCACATGGTGTACAGGAAGGGACT
CCATGAATATACAGGCGTATTATTTTAATGCCATTATTACTAATGTCAGAAGCAAGCATTTGTGCAGCACCAGGTATTGAGTCATTGGAGAATA
ATTCACGAGACACTGTCAGTTTGCAGATTGACAGCTGTCAGGCTGGAGCACTAAATCTGACTGCAGACCCAGAATGCAGGAAGAGTGTGGGCTT
AGCTCAGGTGTGTGCTGTCCAAAACTTCTCTTTCCTTGACCAGAGATATGGGTGAGGGGCTTAACTCCGGCTTGTTTACTAGCAAGCCAGGCAT
AACTGCGGAGGGCCTGCCTGCTTTTCTCGGGACTGACAAAACTGAAGGGCCAAACTCAGGCTTGTCTTCAGGGACTTGGGTGGGACCACTTGGC
ACACATTAGGTGGTTAAGAAAGGTCTGTGGGGTAAATTCATTTTTAAAAAATGTCTATTTAATGTGTGCATGCTTGTGCCTATGTGAGTTTATG
TACATACATGCAGGTGTGAGATGCCTCATGTGGGTCTGGGAATCGAACCTGGGTCTTCTGCAAAAGCAGTAAGCACTTTTTTGTTTGTTTTGTT
TTTGTTTTGTTTTGTTTTTTTGAGACAGGGCTTCTCTGTATAGCCCTGGCTGTCAGCAGTAAGCACTCTTTTTTTTTTTTTTTTTTTTTTTAAG
ATTTATTTATTATTTTATGTAAGTACACTGTAGCTGTCTTCAGACACTCCAGAAGAGGGCGTCAGATCTCATTATGGATGGCTATGAGCCACCA
TGTGGTTGCTGGGATTTGAATTCAGGACCTTCAGAAGAGCAGTCAGTGCTCTTAACCATTGAGCCATCTCTCCAGCCACAGCAGTAAGCACTTG
TAACCCCTGAGCTCTCTCTCCATTTTTTTTTCTTTCATACCACTCTTAACTACCCAGGCATGGTAGGTGCACATTCAATCGTGGAATCTTAAAA
CAGCCATTAGATGACCTAGACAGCTCACTCACTTCTAGAAGCTATCCTGTCCTAGCCGAGTCATAGATGTCATAAGAGCTGTAATCGTACATAC
TCACCTACAGCAGTAGTCATGAGAGATGAGAAACAGCTCCTTTAGCATTCTACAACTTCCCAAGTCCTGGGGGAACTTGTCCAACTGGTGTTTC
TGGGCTTAAAGCTAGAGAGTGATATGGGTAGATGGGATTCTGGCCAAAGCTGTGGGGCCTGTAGCTTCTTCCCTTTCTGACAGGTGTGGGGGTA
TGACAGCTCGAGTAAGGTGCCAGTCCCCACTTGTAGTAGCTCACAGCTGATTCGCTTTATTTCTGGTTTCTAGAATGAGGCTTTGGACAGGGGC
TGTCTGGGGCCATGCGGTCCTCATGCTGGACCTGAGCCCCTAGGATTCCGGAAAGGTTGTACTGTCGTTGTAGCTTTGCGCTTTCCCCAGGTTC
CTTCGGTACCTGAAAGGATGGCAGAGGCATGCTTAGCTAGACCTGGFGTGTGGGTCTCAATCCCGCAGTTAAGTCACAGCATGTGAGCATTTAC
CTAGGGCTGAGCTAGAAAAAGCTCCAGAGGGCAGCCTGGGCTCTCAGTTCTGATCTGACAAGAGCCACAGAGTCTAGCTCAGGTGAAATAGCTT
TCTCGTGTCTTAGTTCCTCTGTTATAATGTGGTGGATTGATTGATTGATTGATTGATTGATTTTGGCTTCTGTTTTTAAAAAAATTAATTGTTT
GGAGGTGGCTAGCACATGTGGAGGTCAGAGGTCAACTTCTGGTCTTCCTGTCTCCACTCAGGCCTTGGGATCACCTGGGTCTGCAGTAGAACTG
TGACTGTCAGTGCTGGCCATTCTGGTGTTCTGCGGCCCTGCTGTGTATACTTGGTGCTTGGGCACCAGTCCTCCGCTAGTATCCCCACACTGCA
CTGCTGCTGTGTTCTGAGGAACCCATAGGTGTGAGTGCCTCTGCACAGCAGAAGTTACCAACTGGTTGGGAAAGGAGAGAGGTAGCTGCTGTGG
GCCATCTGTAGTGGACGGTGCTCATAGAAATCATTTGCCCTGGGGTGAGATAGGAGGTGTCCTTCCGGGACCTGGCCAGACAGTATCCGGCTAA
ACAATACCTCTCTATGGGTGGGAAGGACTCTGGACCCCGTGGTTCAGGGTGAGCTTCTGACAAGATGCTCAGAAACCATTATTATAATAGTAGA
TATACAAGACCAGAGCTTGGTTAGATTCCCTAGCCATAAATATAGCAATGGAAACTGTCTATTGGACTGGCTTGTGTGGGACTTAGTAGAATGC
TAGCCCAGCATGCAGAAAGCCCTGGATTCTTCCTTCAACACTGCAGAAACTCAGTATGGTGGGGCACACACTCAGAGGTGGAGGCAGGAGGATC
AAGTTCAAGGTCATCCTCCGTGACATAGTGAGTTTGAGACTCACTGTGGGCTACATTAGATCCTGTTTCAAAGCCAAACCAAACTGGCTGGTGG
TGCACACCTTTAATCCTGGTACTTGGCGGGGTGGGGCAAGGGGGAGGGCACAGAAGGCAGACAGATCTCTGTGAGTTTGAGACCAGCCTGGTCT
ACAGAGTTTCAGGACAGCCAGGGCTACACAGAGAAACCCTGTCTTGAAAAACGAAGAACCAACCAACCAAAGAAACCAAACTTATTATTTATTT
GTCTGCTCATCTCAGATGATAAGTTGACCTGTCATCTGTCATCTTAATAGAATTCTACTTGTGTTTGTTTCATGGCTTCTGCATGTACCAGCCG
CAGGGATGTATACCAAGGGGAATTGGGTCAGAGAGACTGACTTTGGGGTCTTTTACTTAGAAAGGGAGTAGGATATAGACCCATATGTCCTTGA
TTCCCAAGGCTGAGCACTTAAAAAAAGCGCTGACTGTGTCAACCCCCTATGCCTTGTGGTGCCTGGGCTATCCTGGAGCCCTGGTGTACCTGGC
TGGACCCTAGACCCTGGAGTGCTGCTGGCTGCCTGCATGGCCTTCTCTCCACAGGTGGGTGAGGGAGAGAAGGCACGTTGCCTTGAGTCAACCA
CAGCCTCCCCAGCCTTATAGAAGGCTGTGCTGCTGCTGGTGGCCCAGCTTCCAGAGGGTTTTAAAATAGAAGCAGAGCCTGGAAAGCCAGACTG
TCCCCCTGTGACACTCTGTGGAGGGGGAGGGGAAGGCAGCTGCTTTGGGAACTCAGCCTGCTTCCAGAGCAGAGGCCACTGCCTTGCCAGATGT
GTGTGTGTCTGTGTGTGCCTGTGTTTATGTCTGTCTGTGGTGCAGGGAAGCTCCTGAACTGAGCCTGCTTTCTGCTGAGAGGTTGATTTTTGCC
CCTGGTCCCTAGCGTTGGAGAAGTGGTTGATTTTGCTGTAATCTATCATGACCTCTTTTTTATTTGTTATTTTAATTTTTAGATATATTTATTC
ATTTTATGTATATGAGTACAGTGTAGCTGCCTTTAGACGTACCAGAAGAGGGAATTGTATACATTACAAATGGTTGTGAGCCACCATGTGGTTG
CTGGGAGTTGAACTCAGGACCTCTGGAAGAGCAGTCACTGCTCTTAACCTCTGAGCCATCCCTCCAGCCCCCAACCTCTTGATTTCTTTATATG
TATAAGGGTTTTCCCTGTACATATGTATGCATATCATGCTCATGTAGTACCCACAGAGTCCAGAAGAAAGCGTCCATCAGATCCCCTTTAACTG
GAGTTAGAGAATATTTTGAGCCCCTGTGTGGATACAGGGAAGCAAACCTGGGTCCTCTGCAAAAGCAGCCAGTGCTCTTAATGGCTCAACCGTC
TCTCCATTCCCTTGATACCTCTTTTGTTTGTGGGTCAATTTTTTGCCCTGGCAAACCATCCAAAACTGTTGGAGAAAGAGCAGAGCCAAAAGTT
TTCCCATGATTCCCTAACCTGGGATGGTGATGGTAACATTTGGTGGCTTTGGCTCCTGACAGTAGCTATGTACTGTTAACCGGTTATTCATGCA
GCGTAATAAGGAAGTCACTTGCGGGTGTACTTGAGAAAGGGTGGGCTCTGGGTCACACCCAGGGGCTCTTCTGCCAAGTTGATGACTCCTCCAT
CATCATCTGTAGTTCCCTGGATGGTCTGAGAGATGCCACCGTAGGTGGCTCCTGATTTTTGGGAAGCTGATCTTGTTTCCCCTTTGCTTTTGGA
GATGGGGGTCTGAGCTCACTGCGTGTTTCCAGTGTTGAGGAGACTCTACTTCCTGGTTAGGCGTGAGACCTGTGTTAGCCTGTGTTAGCTTGGG
CTTCTGGTGGGTGCACAGTGCCGAGTGAGGCTAGTCTTTTTTTGTAGTCCATTGCCACCCTCCCTCAGGCTGCTTGATAGCTGCCCTGGCAGTT
TTTATACACATTGAAGATAAGGAGCTTTGCCCTGTTCTTTGCCTTCTGGGCGGAGTGGCCCAGATGTTGGTGGCTGCTCGGGAGAGGAATATGC
CCCCATAGGCCTTGCCCAGTGATAGGGCTACTGTCTCAGTTTGAATCAGTCCTTAGAACCAGTATGAACACTGTCCAGACAACTTTTCCTTCCT
GTATCTTAAAATATTACTCTTATGTCAATGGGTGTTTTGTCTGCTTGTGTGTATATATACTATACCATGTGCATGTCTGGTGTCCTCAAAGGGG
ATTGAACCTCCAGGAACAGGAGTTACAGATGGTTGTCAGCCACCACATAGGTACTGGGAATTGAACCCTGGCCCTCTGGAAAAGCAGCTATTGC
TTTAAACCAACTACCATTTCTCCAGTTCTTTCTCTTAAAAAATACTTTATGTGTACGTATGTGTACTACATGTGTGCAGTGCCCGAGAAGCCAG
AGGAGTGCGTTGCAGCTCCTGGAGCTGGAAACCCAACCCTGATCCTGAGAAGAGTGGGCAGTGCCCTTAGCCTCGGAGCCATCTCTCCAGCCCT
TTGGTGTCTTGACTGTGATGACATGTTGGAAGGTTGCTGACTTCCAATGTTTTCTGCTCTTCACAACACTATTTCAATAATGAAGTAGAATAAA
AAATGGAAACCACATCTGAATTCCGCATCTACTTTTCCCAAACCACATTTCTCCCTGAGGCAGCCATCCTGCCCAGATGGGGCCCCTCTTTCCT
GATTTCTCCCACTCACTCACAAGTTCCTGAATTGATAAAGGACCCACTAATTATGCAGAATTAGAAATTTCCCTGGCATGTTCTTTTCTTTTCT
CTCCTTTTCTTTTCTTTCTTTTTAAAAATATTTATTTTTACTTTTTTAAAAAAAATTAGGTACTTTCTTCATTCACATCTCCAGTGCTATCCCA
AAAGTCCCCACCCCCACCCTCACCCCCACTTCCCGGCCCTGGCGTTCCCCTGCACTGAGGCATATAAAGTCTGCACGACCAATGGGCCTCTCTT
TCCACTGATGGCCTTATGTTCACTTTTTGAGGCAGTGTCTCATTATGCAGCTCTGGCTGGCCTTGAACTCAGAGATCTGCCCGCCTCTGCCTCA
TGGGAGTTGGGGTTTTAAAGGCCCGCACCACAACACCCAGCTTGGCTTTCTCTTTTCATTAGGTGGTACATAATGTTCTATGTTCTCACATATA
AATTCACCACTGTTAATTAATTGTGTTGAGATAGACTCAAAACCCAGGCTATTCGTCCCCTGTATGACCAACAATAACCTTGAACTTTTGGTCC
TCCAGCCTCATCCTAAGCGCTGGAATTACAAGTTTTCTGCCCCCGTATCCATCTCCCCTCATTGCTTTTTGATGACTCTTTGGTGATCCTTAAT
TTGTTACTATCGTACTTTTTTTGGTTATGTCATTTTTCTAATGATGGGCATATTGGTCACATCTAGGTTTGTGTGACACAGGCAATGGGGTAGA
GAATATGTTCTGTAGGTATTTTGGTGCCTAGAACACTTAGTGCGTGGGGTTCCTGGAGTAAGATTAATGCTGAGTTAATTGTTTATCAATTATT
CTCTAAAAAAAGTGGACTGTATTCACATTCCGTTTCCTGACATTTTTCCATTCTTGGGGTCGTCAGTGTACAGCAGCTCCTGGAATAAAACCCA
TAGCTGGTTTTAAGGCCTGAAGCACCCAGGATACCCCCTGAGAGGTCCCTCACCCTGGCCTCAGCTCTTTCCTAGGGGGCTTGCTGCTCTGGGG
ACTCCTGGTCTGGTCTGCTCTGCTCCGCGCTGGCTTGCCAGCTGCCTCTGGGGGGGTGGGGGTGGGGGTAGGGGAAGCGCCCACTTCCTGGTTA
CATTGTAATTTACTTGTTCCTATTGTCAGACACCAAGTTTCTACTTTCCTCTTTTTGTTACAATGATACTTTTTCTTTCTTTCTTTCTTTTTTT
TAACTGAGTCGGGCTCTGGCCTGAGTCCTGGTTCATCTAGAACTCACCTTCTGGACTATCCCTGTTTGTTTTTCATCCTTTTTTTTTCTCTCTC
TTAAATTTTATGTATATGAACGTTTTGTATGTGTGTACACCTGTGAGCCACCATGTTGGCTGATTCTAATGAAAGAACAGCAAGTGCTCCTCCC
TCACCTCTGAGTCATCTCTCTAGCTCCTCCTCTCCACTGTTTGTTTTTTTTTTTTTTTTTTTTTTTTGAGACAAGAGTCTTATGTCGCATAGGC
TGGCTTCGAACTCACTATGTAGCCAGTGATGACTTAGAACTTCTGGTCTTCTTGCCTCTCTCTCCTGCAGGCTGGGATTATTGTCATATGCTAC
CAACCCTGGTTTATGCAATGCAGTGATAGTACTCAGGGCTCTGTGCATGCTAAGCAAGCATTATACCAACGGAGACTCATCCCACCCCTTAAGC
TCGTCTCTGGCCTCAGCCTCTGAAACTGCGGCAGCAAAAAGCACGAAGCACACCCGGCTTGCGGTGATGCTTTGGGAGGGCTGATGAGGTAGCT
CAGCAGTTAAAGGCACTTGCTGCCAGGTCTGAAGACACAGTTTCAGTTCTCAGAAGCTATATGGTGGAAGGAGAGAACTAACTCCTAAGGTGTC
CTCTGACCCTCACAGGTGCACCACGGTACACACAGGCTCACTAAATACACGTAATTAAAGATATAAGATGTGTTACTCACTGCATAGTAACTCA
CGAATGCCCATCATGTGCTGGGCCTTGACTTCTTTATAGACATTTGGGTAAACATATGCTTGACGGGCTCCGAGGAGCTTTGAGTTCAGGGGGT
GTATGGTGGGAGGAGGGGTAGAGGGTGGGAGCACGGTGAGTGAGCAGCTGTCACTGCCATGTAGTTGATGAGTCAGGAAGATGTTGTCCTCCCT
AGGCCCCAAGATGGAAGTTACGTAAGTGGGTTGTGTGAACTGCTAGGTGATACACAATCCTTCCCTCTGCTTCCAGTATGGCTGGGTATCCATT
CCTTAGGCTGGAGTGGCGTTTGCATAATTTGTTCAAGGTCAGCCAGGGAAGGCTTGGGTTGGTAGGCTGGCAGACAGGGGGGCAGGGGAGTCGG
GGAGGAGCAGGTGTCCCCCGAGCCCCCCCCCCCCCCATTGAAGGCAGTATGGTGGAATAGAAAGAAAGGGAGCCTCACTGAGCCTGGCCCCTAC
CCTGAGCTCAGAAGTTCCGACTCACTCCCTGGTTGATAATCTCCTTAGAGCTGATGCAGTAGCTGGTGTTTGCTGTGGGTAGTGGTTTTTTTTT
TTTTTTGTTTTTTTTTTTTCCTCTTTTGACAGGTGGCTTTCCATGCTGGGTTTGAGGATTAGTGTCAACAGACTGCATTTGGGGAACACTAAAT
AATGAATGTTCTGTCTTTTCCTCAAGGTGGGATCTGGTCTAGATGCCTGCAGGGTAGGGGCTCTGGGGGGCTGTCTTTTCTGTGAGGTCAGAGA
AGAGTAGCAGGAAAGACCTGGCTTCTGATAGGCTCAGGTAGACACAGGAAGATCAGAAGTTCAAGGCCATTCCTGTCTGCATAGTGAGTTGGAG
GCTCGATTGGGCTACATAGGACCCTGCCTCAAAAGGAAAAAAAAGAGGCAGGTGTGTAGGCACACTTTCAAGGTTAGCTCAGGGGTTAAGTAAG
AGCACTGTATTTTTGTTGTTGTTGTTGTTGTTTGTTTGTTTGTTTTTTTAAACTTGGACCTGCAGTTTTGTTCCCATGACTACCTTTTAACTCC
AGCTCCAGGGGATCTGGTGCCTCCTTCTGGTCTTTACAGGCACCTGCACACAAGTGGCACACACAGATATATATTTATATACACAAAAATAAAA
TGAAATCTTAAATATTGGTTAGAGTGTGTGCATGGGCGCTTTAGTGTTCTTGGATGCTGCAGATGGGCCCGCTCCCCCTCTGATCCTTCCTCTA
GGGGAGAGACAGCTCTGTCTGTAGATGTTTCTTTTTGTTTGCTTTGTTCTGTTAGGGGTTTTCAGGTTTATTTGATTTTATGTGTAGAAGTGTT
TTTGTGTGTGTGCGCGCGCTGGGTCCCCTAGAACTGGAAGTCATGGACAGTTGTGAGCCACTGTGTGGGTGCTGGGAATTGACCCCTGGTCTTC
TACAAGAGCAACAAGAAGTCTTAGCCAGGGAGCCATCTCTCTTGTTTTTGTACTTTCACGGATGGCTCTTAATCCAGCAGAGATGGAGAATGTT
CTGGGCTCTCTGCCAAGATTTTCTTCTGCAGGTCTGCTGGGCTGGGCTGCCTTTCAGCCCAGGATTGCTTGGGGGTGACTTTTCTTAGGGGAAG
GGCTAGGCCCATCTGGCCTGAGGGGGAAGGGGAAGAGGGGAGTCCCCAGCTCTGGAAGAGCGGGTGGGGCAGTATTGGGTATATCTCTGCCCTC
TCTTTTGACCTGACCTTAATGTGGGCTGGTCTGCCTACATTAGGTTCCAGCCTGTTGGTGGGCGAGAGGTAAGAGCCGCAGCAACACAAAGAAG
GCTCGAGCAGTCTCTGAAGATGTGAACCATGGCCTGGGCCGCAGATTCCATTCACAGTGGAGGCTATGGCTGTTTCTTCTGTAGTGTTTATTGG
GTTTGGCCGTAGGCTGTCTTTAGTTATTTACTGAAGGATTGGTTTATTATGTGGAGGTAACTTTTGGGAGTCCCGTTGCATCCTGCAGGTTCCA
GCTGTGCCAGCAGGCACCTTCACCTGCTGGATTACCTTGCAGGTCCTGCTGTGAGCTCTTTGTGGTGTCCTTGATTCAGGTGTTGGTCAGTTGA
TATTGTTTCCCTGGAATCATTTGAAATCTTCAGTGGGCGCCCACTCGCCAGGAATATACCATAGTATATTCGTGTTTGTGAATGGGTACATGTG
TCATGTCAGATGTCGCCTATGCAGATCCAGTTTGATAAATTAGGTGTACAGTTTTTGTTGTTGTTGTTTGTTTCTTCATTTGTCTCCTCACCTC
TGACACAAGGTTTCTTTGCATAACAGACCTGGCTGTCCTGGAATTCACTTTGTAGACCAGGCTGGCCTCAAACTCACAGAGATCCACCTATCTC
TGCCTCTTGAGGAGCTAGGATCAAAGGTGTGTGTCACCCTCACCTGGTAGGTGTGCAGTATTTTAAATGAAGGGAACCAGCTGAGGATGTAGCT
CCACTGGCAGAGTGCCTGCCTGGGGACTTCAAGTCCTAAATTTGATCCCAGCAGTGCAGAAACTACATGTGGTGATGATGCCTGCAATTTCAGC
AGTTGAGGGATGGAGGATTAGAAATTCAAAGTCATCCTTGGCTACATAGAAAGTTCCAGGCCATCCTGGACTACATGAGATCTTGTGTGTGTGT
GTGTGGGGGGGGGTTGTGTGGTGGGAATGGAACCACTCTTCTGGTTAGTCACATTATGGTTCTGGGATGGGACTCTTGTCACTCATGGGTCCTT
CTCTTCAGGACAGAAGTCACAGTGTGTGCTGTCCCAGAGCAAGGTTTAATTTTTTCTACACAGCTTGTCAACATAAAACCTTCCAGGAATATAA
TGTCACAGAGACCATTAGAGTTATTCTCTTTCCCTGACCACTGTGATATTTAACTAATATTTGCTCACTAACTTAATTATGTGTCACAACTAAG
TCCTGCTTCTGACCTCCTAGGGTCCCAGCACCCATGTGTGTGTGTATTCACTCATAGATACGTAAATAAAAGAGATGATAGCACTTTTGATGGA
ACCTGGAGCCTTTCATATGCTAAGCAGATGTGCTACCCTGCACTCTAGTCTCAGCTCCCTGGAGAGTATTTTTTTTTAAGATTTATTTTATTTA
TATGAGTACATTATAGCTGTCTTCAGACACACCAGAAGAGGGCATCAGATCCCATTACAGATGGTTGTGAGCTGCCATGTTGGTTTGCTGGGAA
TTGAACTCAGGACCTCTGGAAGAGCAGTCAGTGCTCTTAACCGCTGAGCCATCTCTGCAGCCCCCAAGTAGTATTTTATGTGCATTTGGGTGTT
TTGCCTGCATGTGTGAGGCTGTTGGATTCCCTGAAACTGCTGTTGCAGACAATTGAGACCTGTCCTATTTGAGTGCTGGGAATTGAACCCAGCT
CCTGGANNNNNNNNNNNNNNNNNNNNTGGCTCACATACATACATGTGTGAACGTGGTTCACATATATACATGCGGAAACACATAAGGTGAAAAA
GAAGGGGCTGGAGAGATGGCTTGGTGGTTGAGAGTACTGCTGCTCTTCTGGAGGTCCTGGGTTTGATTCCCAGCACCCATGTGGCAGCTGAGGT
TGTCTGTTACCCCAGTTCCAGGACATCCAACACCCTCACACAGATGTGTGCAAAACATCAATGCACATAAAATCAAAAGAAATCATTAAAGAAA
AAGCCAGGCAGTGGTGGCACGTGCCTTTGATCCCAGCACTTGGGTGGGCTTGTGGGAGTGACAGGTGGATATTTGTGAGTTCGAGGCCAGCCTG
GTCTACAGAGCAAGTTCCAGGACAGCTAGGTCTACACAAAGAAACCCTGTCTAAAAACAAAAATCAAATCAAAACAAAACAACAGCCCCCCCAA
CCCAATATTTAAAATTGTTTTGTGTGTGTGTGTGTGTGTGTGTGTGTGTGTGTGTGTGTGTCTGTGCTCGTGCACATATGCACACGTACATGGG
CACATGCATATAGAGGCCAGAGGACAACTTGCTGGGGGAACTATATCATTTTCTCAGGGATTGAACTCAGGGAGCCTGGCTTGGTAACAAGTGG
CAAAAGAGCCACTGAGCCCTCTTGCTGGTCCCTTCTTTTGTATCCTTGTAGTTTGTTTCTTTCTCTCTCTCTCTCTCTCTCTCTCTCCCTCCCT
CCCTCCCTCCCTTCCCTCCCTTCCTTCCTTCCTTCCTTCCTTTCTTTTTTTCTTTTTTTGAGACAGGGTTTCTCTGTGTAGCCCTCGCTTTCCT
GGAAGTCACTCTGTAGACCAGGCTGGCCTCGAACTCAGAAATTCGCCTGCCTCTGCCTCCCAAGTGCTGGGATTAAAGGCGTGTGCCACCAGGC
CCGGCTATGATACCCTTCTTTTGTAGCATGTTGTTGGACTTCATTTCCTGTGAGGAGATACTTAGCTAGCTTGTCATGCTGTAAAGAAAGTTTT
GTGGAGCTGGGCTTGCAGCTCTGATACTTGTTGAAGGAATGAAATCAGAACCATGTGAAAGATACATCGATTGCCATGGGAAGCTTCAATTTAT
CCGTGTGCAGTTACCAGCTGGGCGGCCGGCATTTTGTAATTACTGGCAAGAGTTCTGCGGTTCAGTATAGTGGCTGACTCCTGGGAATATGTCT
TGGATGGTTCATTCATTCATCAAATATTGACTGACTCCTTGTGCTCTGGGTCGGTGCTTGGGCCTGGCGTTGCTACAGTCAAAAGCAATCACTA
AGTGTCCCCAGCTCTAGCCTCAGGGGAAGGGTGAGACAACTCAGGGCACACCCACATCTGGCACACCCACATCTGGATGCCTGAGACTTAGAGC
TGAGTGATTTTTATTGGTTGTTTAATTATATCAAATAAGTTTTATTGGTGGTAAACTAATTTCCATAATTACCTATGTGTTCATTCTTTCTCGT
GCTTTTGTTGTGGCAGAACATGGCCGTGCCTCCCACATACGCCGATCTTGGCAAGTCCGCCAGGGATGTCTTCACCAAGGGCTACGGTGAGTGT
CGGAGCAGTCGGGAGCAATGGGAAGCCTTTCACCTACTAAGGGAGTTGGCATCTTTGTGACACTTGAGGTGGTCAGTTCCCAGCTGACAACTTA
TATGTCCAGACAAAAGGATGAGCTTCCTGTGGCTGTGCGCCACCCAGTGGTGAGGCTCAGGCATCACAGGGGCAAATGGCAGATCTAGTCCCTT
GCAAGCTCCAGCGTCTGCCCCAGTAGAACACTGTTCTAAATTTACCCTGATGGTCCCACCTCTGATGACTGCCTCTGCCAGCTGCTGGAACCTG
CCCCACAGNNNNNNNNNNNNNNNNNNNNCTGCAAGGGCACAGGTCAGGCCTGTGTTTGTGTCTGCCACTGTTGCCCTGGCTTCCTTTAAGTATT
TTCAGATAGCTAGGAGGAAAACAGTGGTGTCCTAGCTTCAAGCTATTATTTTGATTATTTTTTTTTTTCCTCAGTGCTGGGGTCAAACCTTTAG
CTTGCTAGCAAACATTCTGTGGCTGAGATACACCTCCAACTCCTGGCTTAATTAGTTAACCTGATTTTGGCTGTTTTTCCTTCTTTAGGCTTTG
GCTTAATAAAACTTGATTTGAAAACGAAGTCAGAGAATGGATTGGTAAGTCTCTCCACTTGCACGGGACGTTATATTGTCCCTTACCAGGAGTG
GGTACTGTGGCCCGGGTGGCAGAGCCAAAAACAGTTGCCTGCTTGGATTGTGGCTGTTAGTGCTTGTCATCGAAGCGCTGTTCCATAGGTGGTC
TGGGACTATAGTAGCTTTTAATGCTTTTTTTACCAGTTATTTTATGTTGTTTGAAATCAAAACTTCTGGCCTAATGTACTGTAGGTGTGTGCTG
TACCCACTGATCTACCCTGATCCATCCTTGATGCTTCTTATTTTAAATTTGACCTCTCCTTCCCCCGCCCCCCTCTTATTTTGAGGTAGAGTCT
TACTGTGTAGTAGCTCAGGCTGGCTTCAAGTCACAGAAACTCTCCTGCCTTAGTCTCTCATGTGCTTGGAGTATAAGATGTGAGCCATTACACC
TAGCTTTGACCTAGGTTGTTTTGTTTCTTTATTTAAAGACTTAATTTATTTCTGTGCATGTGTGCGTGCCTGCAGGAGTTTGTGCACCATATGC
ATGCAGGAACCTGGGGAGGCCAAAGGAGGGCATTAGATTCCCTGGACTGGAATTACTTTTATAGGCCATTGTGAGCTGCACAGTGGGTGCTAGG
ACTCGAACCCAGGTCCTCTATAAGAGCAGTAAGTGAGTGCTCTTAATTGCTGAACCATCTCTTCAGCCCCGCTGACCTGTTTTTTTTGTTTTTT
GTTTTTTGTTTTTTTTTGTTTTTGTTTTTTCGAAACAGGGTTTCTCTGTGTAGCCCTGGCTGTCCTGGAACTCTCTCTGTAGACCAGGCTACCC
TCGAACTCAGAAATCCGCCTGCCTCTGCCTCCCAAGTGCTGGCATTAAAGGCGCGCGCCACTACCGCCTGGCTTTTTTTTTGTTTTTTTTTTAC
TTATTCATTTATCTTTATTTTTTTGAGACATTGTCTATCCACATGGCCCTGGTTGCTCTAGAACTCACTCTGTAGACTAGGCTGGCCTTGAACA
CACAGAGCTCCACCTGTCTCTGCCTGAGTGCTGGGATTAAAGGTGTATGGCACCACCTATGTTTGTAATGTTTCATTATTATTTAGGAAACATT
TCTGGTGTTGATTGATACAGATACAGGTATCTGTAGCAATTGTTGGAGGGAAGGGGTGTGTTGCTCTGTCAGTAGTAGCTCCCTCTGAGGCTGG
AGGCAACATGGAAGCTGGGTGGTTTCCTGTTCAGACTTTAGGGCCCCCAAGGGAAGAAGGAAGCTGTCTTCCAACAAAGACCTTTCCAGTAGGT
TTCCTTGGGTAGCAGACCTCTCACTCCTCTGTATTTCCAGTATGTAATTGAACTGTAGAAGTAGTTCTGCCTGGACACGTTGGGGACTCTCTGT
TGCTTAAGGGCCTGGACCAAGGGCAGACTGGACTGTAGGCTAAGGTCTCTATCTCTCTCTCCTTTAAGGAATTTACCAGCTCAGGCTCTGCCAA
CACGGAAACCACCAAAGTGAACGGCAGCCTGGAAACCAAGTACAGATGGACTGAGTATGGGCTGACGTTTACAGAGAAGTGGAACACAGACAAC
ACCCTGGGCACTGAGATCACTGTGGAAGACCAGGTAAGAGCTCCCTGCCTGCTGGAAGTCCTGGCTCCTCTAGTTCCCATTGAGACATCCTGGG
TCCTGTCTTTGGTTAACCTGTGCCACCTTTCTCTTATTTGCAGCTTGCTCGTGGACTGAAGCTCACCTTTGATTCGTCATTCTCGCCGAACACT
GGGTAAGGTTCTCCTTACCCACGAGGATATTCTGAGATGTTATGGGCCCTGGAAGTCCATTACAGCAAAGGATTTTTGGGCTGACATCATGCCA
AAGGTAGCCTCTTAGCTGGGAGGCTGGAGGGTGGAAGGAATCGTGTGGTCAGGACCAGCCAGGGGTATGTAGTGAGATTCTACATCAAAGAACA
AAAAACCAACTAGGTAGCAACAACAGTAAGGCTGCCTTGGTCCAAGTGCTACATATTCTGGTCTGTCGTGGTGTTCTATCCAGGACATGAAGAT
ACAACTGTTAAGGGACAATGCTGTAGCAAGCATTTAAAAAAAAAAAAGATTTATTTATTTTATGTATGTGAGTACACTGTAGCTGTGTAGAGAG
TTGTGAGCCATCATGTGGTTGCTGGGAGTTGAACTCAGGACCCCTGCTCGATCTAGCTCTGCTTGCTCCAAGCCCCGCTCGCTCTGACCCAAAG
ATTTATTTATTATTATATGTAATTACACTGTAGCAGTTTTTAGACACACCAGAAGAGGGTGTCAGATCCCCTTACAGATGGCTGTGAGCCACCA
TGTGGTTGCTGGGATCTGAATCCAGGACCTACAGAAGAGCAGTCAGTGCTTTTAACCGCTGAGCCATCTCTCCAGCCCATTGCAAGCATTCTTA
ATTTAGGAAATGTATTTCTCTAGCAAATGGTTTTGTGTGTTCCTGTGCTCATTTGGAGACTGAAGGTGTCTGAGCTGGGATACTATTTGCCCCA
AGGAACTTCCCTTTTCTTTTTGTCACAGGGTCTCCTATAGCTCAGACTGGCCTTGAACTTATTTTCTAGCCAAGGGTGACATTGAACTTCTGGT
CCTCCTGCTTCTGTCTCTTAAGTCCTGGGATCATGGGCATGTGGTCCCATACCCACTTATGGGGTGGTAAGGATGGAAGCCAGGGCTTATGCAG
TCCATTTGTGACGTATGCTACGTCTACACCGAGCTGTGTCTCCAGGGTTAAGGATTGTCCTTGGTAGTGGAGGACTCAGGCCAGTGTTTACTGC
TAAGATCCCACAAAGCCACTGTGTGCCTGGCTGCAGGCCACGGGCTCTCTGTCCCAGCTGATCTGTTTCACATAAGGATGCCAGTCAATGAAAG
TGGCCAAGGCTGAAGGGCAGAGGTGACATTTGGAATCAGGGGCTGACTGGTCACTGTTGCCAGCCTATTTTGTACATGTGGAGGGCTCTTCTCC
CAGTTTTCTGAGACCCTGAGATCCTGAGAGCTTAACCACAAGGTGGCTCCTGGGGCTGCCAGGTCCTCCGGCCAGAAATAGTTTAGGCTTGTTG
GTTATTGACACATCAGAGGACAAAGAGAGCAAGCTGGTCTGCCTCTCCTGAGTGCTCCACAGGGCAAAGAAGGAACTCTTCTTTGAGTTCAGGC
TTTTACTAAGTTTCAAGCTTTGAGTTTTTGATTTGGCAGGTGTTTTGCTTGTTTGATTTCTTTTTTGTTTGTTCCCTTTTTTTTTTTTTTTTTT
TTTTTNNNNNNNNNNNNNNNNNNNNATAAATATTGGTAGTTGACATAAATCCTTGCTGAGGGACAGTGGGTCTTGAGAAACAGCTGACAGACAC
TGAACTAACTGGGCTTCGATTTAAATGTGGGCTGGACCTGAGACCTGAAGGACTCAGGGTCCTCATCAGTATCCAGAACTCTTGAGTTGTGAAC
TCAGTGGGGTCTCACGACTATGGTGGCAAAAGGTCTCCTAGCATAGTCACTAGGCAAGGGAAAGACCCATTGTTTATTTTATGTGTATATTCCT
GAGTGTATGACTATGTATCACATGTGAGGGGGAGGGGGAGGGGGGACCTGAAGGTGTCGGATGCCCAGGAACTGGAGTTACAGGTAGCTGGGAG
CTGCCAGTGTGAATGCTTGAAACTGAATCTCAGTCTTCTGCACTCTTAACTCTGAGCCTGATCTCCAGCCCCGGGGTCCGCTCTTTCCAGCCTG
ATCTCCAGCCCCGGGGTCCGCTCTTTCCTTTTTCGCTGGCTACTGGGTGAACCACAAAGATGACCATCTCCCTCAAACTGCTGTGTGGGTAGCT
TTGAGGTACATGCTTCTTTTCTTTTTTTTTAAAGATTTATTTATTATTATATGTAAGTACACTGTAGCTGTCTTCAGACACTCCAGAAGAGGGA
GTCAGATCTCGTTACGGATGGTTGTTAGCCACCATGTGGTTGCTGGGATTTGAACTCTGGACCTTCGGAAGAGCAGTCGAGTGCTCTTACCCAC
TGAGCCATCTCACCAGCCCCACATGCTTCTTTTCTGTAGGTTTGGAAAACAGCTGAGAGGCCTAATAGATGCCTGTGTCAGGTTTTGGGTTAGC
CGTGGGGAGAGGCTGGAAAAAGAACATGGTTGGCACTCACTTTTTGATCTCTTCCCATGGTGTACACAGTGCTGATGCGGCCTTTTTCAGACCG
AATTTGAGCTCAGGAGAAGTTGTGTCCTGTTGGTGGCTTAGCAGGTGTTTAGGGACAACTGGCCACTGACCTCACTTCTATTTCCTGGCTTCTA
CACAAACTGGGAACAGGCAAAATGTTTACCGTTCTTGCATGATTGTATCGAGATGAGTTAGCTCGGCTTTAATACACTCTTCCTTTTCCCTTTA
GGAAAAAAAATGCTAAAATCAAGACAGGGTACAAGAGGGAGCACATCAACCTCGGCTGTGACGTGGACTTTGACATCGCTGGGCCCTCGATCCG
GGGCGCTCTGGTGCTTGGCTATGAGGGTTGGCTGGCTGGCTACCAGATGAATTTTGAGACCTCGAAGTCCCGAGTGACCCAGAGCAACTTCGCA
GTTGGCTATAAGACGGATGAATTCCAGCTTCATACTAATGTGTAAGTGTATGTGGGGGGTTGTTTTAGTTGCTTTCCTGTTGCTGTGACAGAAT
ACCACGACCAAGGCAACCCGTGAAAGAAAGTGTTTGGTTAGACCCACCGTTTTAGTGTGTTACAGTCTATGAGGGCAGCAGGAACNNNNNNNNN
NNNNNNNNNNNCCCACCTTTTGGCATCCATGGTTCAGCGGCATGTGCGTTTTTATCTTTTGTGATTCTGGGCTTTTTTTTTTTTTTTCTTTTCT
TTTTTTATTGCCCACCTAAGCCTCTTCAGTGCTGGGATTAAAGGTATGAGCTGCCATGTGGGTGCTTGGAACTGAACCTTGGTTCTCTGCAAGA
GCAGCAAGTGCTATCAACTGTTGAGTCATCTCTCCAGCCCCCTGAATTTAACCTTTTCTTTTGAAATAGTTTTAAAGTTTTTTTATAGGGAACT
GTCATACAAGCAGTATGTAGAGCTCTCCTCTACTTCTCCCCTACCTCCCTCTGATGTTGAAGTTTTCTATCTGCCATCACATTATAACATACTA
TATAATATACAGCTATTCATTACATAATAATATTTCAGTCAAGCACCTATAGGACTGTGGCCTCTGAGATTATTACAGAGCTGAAAAATTCCTG
TTGCTTATTGCCATCAAAACACAGTGCATTTATTGAGTCTGTCAAAATTGACATGAACACCACCCACTCTGTTATCTCCCCATAAATGTGGAGC
AGGTATAATTTTGCTCATGTAACAGTATTTGGACCCAGTGTGTGCCTCTCTTGTGGACTCACTCCCTGTGTTCTTACCATTAGAGCATGTTTTT
TTTTTTTGTTTTTTTTTTTATATAAAATATTTACTGTGAACCCACGTGCTATGTTACGTGGGGGTGCCTTGGACATCTGTCTCTTGGGTACATG
AGGAGGTCATGGTTGAGTGGTTAATCATGGTCATCTAGGTTTCTGTAAGTGCACCCTGTGATGTTTGCTTAATGACCAAATGGCCCAACGATGC
AATTTTCAGAATGTGTCCCTGTCATTAAGCAATTGTGACTAACAAAGATCCATGCCATCAAGTGACCATTGGTACCACACTATTAACTGATGTC
CTGCTGATATCATCTCTCTGGCCTGGAACCTAGTTCTGGATCCTCTATTGCATCAAGCTCTCAGGTCTCCTTTGTGGCCTCTCCCTGGGACACA
CTCTCCATCTTTCTCTGTCTCTCTCGACCCTGACATTTTTTGAAGAATTGCTGAGAGCTCTTTGCAGAGTGTCCTTCTGTTTGGATTGGTCTGA
TATTTCCTCCTGAGTGTATTCAGTTGCATCTTTTTGGCAGCTGAGGAAGCGGCACCTCAGTGTGCTGTCTGGAAGGCCACTATTGTGTGTCTTG
GCCTGTCATACCAACCCTGATTATGCGGCTAAGGTGGCATCTGCTGGATCTCCGCTGTAAAGCTGTCAGTTTCCAGCTTTGTGCTTAGTAGCTG
TGTTACGGGGAGATACTGAGACTGCAAATATTCTCTCTCATCACACTTTGGGTTACATGGAAGCATCCTTTGATGCTTGCCTGTGCAATTATTG
GTAGGTGTTCCCTTCCTTCTTTTCTTGTTTCTTGAGACAGGGTTTCGTTCTGTGTAGCCCTGGCTCGATCTGTAGACCAGGCTGGCCTCGAACT
CAGTGATCCTCTTGCCTCTGCCTCCCAAGTGCTGGGATTAAAGGTGTGAGTCACCAGGGGAGCCATCATTCCTTTCTTACTTATGGGCTTTTAC
TGTAGGGAAGGGCAGTCCTACTCCTCCCTTAACCAGTTTTAGTTTTTATTTTGATGTATTATCTTTATATTTTGTCTATATATGTGCTTTGTCT
ACATATGTGTGTATGTATGTATGTATGTATACCGCTTATGTGCCTGATGCCTGTGAAGGTCAGAGGATGAGNNNNNNNNNNNNNNNNNNNNTTA
GCTCGGCTTTAATACACTCTTCCTTTTCCCTTTAGGTAAAAAAAATGCTAAAATCAAGACAGGGTACAAGAGGGAGCACATCAACCTCGGCTGT
GACGTGGACTTTGACATCGCTGGGCCCTCGATCCGGGGCGCTCTGGTGCTTGGCTATGAGGGTTGGCTGGCTGGCTACCAGATGAATTTTGAGA
CCTCGAAGTCCCGAGTGACCCAGAGCAACTTCGCAGTTGGCTATAAGACGGATGAATTCCAGCTTCATACTAATGTGTAAGTGTATGTGGGGGG
TTGTTTTAGTTGCTTTCCTGTTGCTGTGACAGAATACCACGACCAAGGCAACCCGTGAAAGAAAGTGTTTGGTTAGACCCACCGTTTTAGTGTG
TTACAGTCTATGAGGGCAGCAGGAACCACTGAGAGCTCCCATTTTGATCCACAACCACAGGGCAGGCAGAAAGAGGGAACAGTGGGGGTGGTGG
GAGACTTGTGAAACCTTCTCCAGCAAGGCCACACTTCCTAATCCTTCCCAAACAGTTTGACCAACTGAAGACCAAGTATTCAAGTGCAGGCCAT
GAGTGTTTATGTGTGTGGCTAGGGTGGTGGCTTTTCTTTTCCTAAGTTTGGTTTTCAGTCATTTGGGTCAATGGGCCCCACGGCCTGTGAGCAT
TTTTGTTTCCCGTCACACTGAGGATAATCAGGCACTCATTCTCTCCATTAACTCGGTTAACAGCTTGCTGGACAGTGGGGTGCTGGGTGAAGGG
CTGGCCTATAGGTTGGGGAGAGTCTGTGCGTGGTCTTGGAGGTTGAAGGACATTGATGACATGAGGTAACAATTTTAGTCATAAAGACAATCTG
ATTTGGGGCCGGAGGAAAGCTCAGCACATGAGAACACTTGCTGAGAGGATAAACAGTTTGGATCCCAGCATTCCTGTCATGCATGGCTCACAAC
TCCCAGAAACTCTAGCTCCAGGGGGTCCATTGCTCTCCTCTGGGATTGGAAGGGGACATCCCCCCAGGCCCCATACATATAAAACTTTTTTTTT
TTTTTCAAAAGTAACTTTACTTGGGTTAATTCGATGAAGTACTAACCTGTTCTTTCCACTAGAGGGCGCTAAGTAAATAGCTTCTTAAATTCAG
ACCAATAATGGTATTAGTCTAAACAAATACTTTTTCCATTAATAACAATTAAAAACATGTATATGTATGTATGTGTGTGTGTGGGGTCTGTCTG
TCTGCTTGTCTGTCTTTCTGTCTGAACTCCTATGCCTCAGTGTAGGTGTGGAGGTTACATAACAACTTTCAGGGAGTCTGTTGTCTTCTTCCTC
ATCCAAACCACAGTGCTTGCCTTCCAAGGAAAGTGGCACAGAACTTTCTTGACCATTCTGGCTGTATCAGCTTCCCTCCTACCATCTTTGACAC
AACCATTAACCAAAGTGAATACACAAACACAGTAGACAGCAGAAAACTCTGTAGGAGTCTAATTTTCTAGTTTCTGCTTTCATTTGGAAAAAAC
AAAACAAAACAAAATGGTTTATTTTTGCTCACTGGTGTGTATGTGTGTGTGTGTATGTGTGTGTGTGTGTGTGTGTATCTGTGTGTGTGTGTGT
CACATGTGTGCAGGTGCCCATGATGGCCAGAGGGGGTTCTGGATGCTCTAGAGCTGAGTTATGGGCAGTTGTGAGCTGCCTGATGTGGGTGCTG
GGAGCTGAATTCAGAGCCTTTGGCAAAGCAGCGAGTACTCTTAACAGCTGGACATCTGTTGCTATGCTTACTCACAGGAATGACGGGACAGAGT
TTGGTGGCTCCATTTACCAGAAGGTGAACAAGAAGTTGGAGACTGCTGTCAATCTCGCCTGGACTGCAGGAAACAGTAACACTCGCTTCGGAAT
AGCAGCCAAGTATCAGGTCGACCCTGATGCCTGCTTTTCGGTGGGTACCCGAGAATCACCCATCTCACACCAATTCTATGCTTCTGCTTAGACA
TTCTCTCACAGGGCAAAGACAAGTCTTGTGTTTGAGATACTTGAGACAGATACTTTTTACAAAGTCTCACTATATGGCCTTGGGTGGTCTGGAA
CTTACTCTGTAGACCTCACAGAGATCCACCTAGTCTGCCTCAGCAGCGCTGGGATTAAAGGTGTGCGCCACGATATTGACCTCCATGGCTTTTT
GTTTTTGTTTTTTGTTTTTTGTTTGGTTTTTTTTTTTTTTTTTGAGACAGGGTTTCTCTGTGTAGCCCTGGCTCTCCTAGAACTCACTCTATAG
ACTAGAATGGCCTCAAACTCAGAGATCTGCCTGCTTCTGCTTCCTAAATGTTGGGACTAAAGGCATGTCAATTTTGCTTGACACCTGCAGTTTT
TTTTTTAATTTACTTATTATTTATTTATTATATGTAAGTACACTGTAGCTGTCTTCAGACACTCCAGAAGAGGGCATCAGATCTCATTACGGAC
GGGTGGTTGTGAGCCACCATGTGGTTGCTGGGATTTGAACTTTGGACCTTTGGAAGAGCAGTTGGGTGCTCTTACCCCACTGAGCCATCTCACC
AGCCCACCTGCATAGTTTTTATATCTTCAGAATTTTCACACTGGAGACGCTGAACGCTTATGGAGGTCGATTGGGTAGCTTCCTTAAAGGCACA
TAGCAAGTATGTGCTGGGACCTGGCTGTAAGTAGACCGCTCTGCTATTGAGCCTCTCTTACTTAAGTGTTTCACTCTTCCTCTCCTTTCCATTT
GGTTTTCAGTCTCTACATCCACATTAAGACATCAATGGTCAGGATGATGTATTCCAGTGGTAGAGCCCTTGCCCAGCATGTCTGGGCTTCAGCC
ACATTTCTGGGAAGAACCCAAACAAACAGAAACAAGGCTGGGATGTAGCTCAGTTGGTAGAGTGCTTGCTTAGCATACAGAAAGCCTGTGCTCG
GTCCCCAGAACTGCGGAAACAGCAGGCAGAAGGATCAGAAGCAGGCCATTCTTGCCTATGATGTAGCAAGAGTTCAAAGGCAACTTTGGGCTAT
ATAGTGAGATCACATCTCTAAAAGAGACAAAACCAGGTGGCCTCTTGTCTTGCTTACCCCGTGGTACCCATCAGGGTTCCGGAATGGCTCAGGT
TTTGTTGTGAGCCTATTTGAGCTGGCTTGTGTTATCACTCTGTAGGTGGCCACCTTTTTGAGGTGGGCTGAGGAAGTGTCATGCTGCAGAATGC
TTCTGCTGGTTGGTGTGCCTCACAGAGAGTCTATTTTAAGTCTCCTGACCTGGGAACTGAGTGCTCTCAACATGAGGCATGCATATAAAACTGA
AAGACAAAGGCTCTCCTTGTTGGGATGTTGGGAGCATAAAGGAACAGTTAGAAGGGACACGGCCTGCTGAGAGCCCTTCTTCTCCTCGCTCTGT
CCTTCAAAGTTCACAGGAGCAGGAAAGCACACACACTTGCACATGACACATCTCATTGAGTTTTACCAGATGGCCATGAGCAGACAGGAACACA
GTTCTGATACCTGTGACTTGCCTAAGATACTGTCACCAAGTTTGGAGGTACACCTGTGGGACAGTTTTCATGGGTGAGGAACACTGTCTTCCCT
TGGACTTAATCTTTTTTTTTTTTTTTCCAGGCCAAAGTGAACAACTCTAGCCTGATTGGCTTAGGGTACACTCAGACCCTAAAACCAGGTATGT
GTTGTTGCCCATGTGCAAAGCTCGTTCTCGGTTGCATTTTGCTGATGTCTGCTATTGCTGAGGGACATTATAAACGAGTATATATAAGCTTTTA
TAATTAAATGTTTATAAAGGAAGTTGTGAAGTCATTGTAAAAAGAAAAAGAGAGTCTGAGAAATAGAAAGCAGAAGGAACCTGTCTGGGCTAAC
TCACATCACAGCTCTAGTTATTTTCTCCTTTCCTTTCCCCAGTATGTGATATGTGATAGCTGCTTCCAGACTGTAGTCAAAACTAGCCTGTCAG
TCATCCTCACGATGGAAAGACTAGGGTATAGGGATGTAGTGGGTCTTCTATGCATAGTTATTCCATCCTCTCACTGGTGTGTGAGGGCTGCAGA
AATCCCACTCAAGGCTGACTATGTAATATGAAAATCAAGTTCTTCCAGGGAAAACCATCCCGTTTGATCTAAAATTACAAGTAAGCCCCAGGAA
AAGAAGTTTATTGTTTTTTTTTTTGTGTGTGTGTGTGTGTGTGTGTGTGTATGCTCACGTGTTATGAAAGTGAATGTAAGACTCAGGCTGTTAC
AAGATAGGGTTTCCCTCCAAGGCAATACTGCTCAAGCAACTTACTGAGTTGGGGCAGCAGCGGATGGCACACTCATTGTAATGGGACTGCAGGA
GGCTGGAAGGGAGGGGGATTCGGAAGTACGTTCCAGCAGGGTCTCTGCTCTGTGTGCCAGTGATGGCCGTTGCATGTGTTGATTCGAGGATAGC
AGCAGGTCAGGTTGTATTACAGAGCACACCTTGAAGGGTCGCCTCGCTTGGCCACTGCTGACTGCACCTGGAACAGATTCTGACCTTGATTCTG
ACCTTGATGCTAACCTCTCTTCTCTTTCCTTTGTCTCTTGCCCCTTTCTCCCTCCTCCGTCCTCTGTCACCCCTCCCTCCCACACCTCTGTCTC
AACTCCCCACAGGTATCAAACTGACGTTGTCAGCCCTGCTCGATGGCAAGAACGTCAATGCGGGTGGCCACAAGCTTGGCCTAGGACTGGAATT
TCAAGCATAAATGAATATTGTANNNNNNNNNNNNNNNNNNNNCTGGCCTCGAACTCAGTGATCATCCTGCCTCTGCCTCCCAAGTGCTGGGATT
AAAGGTGTGAGCCACCAGGGAAGCCATCATTCCTTTCTTACTTATGGGCTTTTACTGTAGGGAAGGGCAGTCCTACTCCTCCCTTAACCAGTTT
TAGTTTTTATTTTGATGTATTATCTTTATATTTTGTCTATATATGTGCTTTGTCTACATGTGTGCATGTATGTATGTATACCGCTTATGTGCCT
GGTGCCTGTGAAGGTCAGAGGATGAGCCTATAGTCCCTGGAACTGGAGTTACAGATCTGTGAGCCACCATGTGGGTCCTGGGAATGGAACTCAT
GTTTTCTGCAAGAGCAGCTAGTGGTGCTCTTAACCGCTGAACCATCCATCTCTCCATCCCTTTATTATCTGAAAAAAAAACACATACACATAGA
TATGTATATATGCACACACATAAATACACACACACACACATGCAGTTTCTTCTGTGTGTGTGTGTGACAGTGTATGTATGGACATCAGAGAACA
ACTTGGCAGGTTTGGTTTTTTTCCTTTTTCCATGCTAGTCCCAGGGATTCGCTCAGGTCATTAGGTTTGCCTGCAAGCACCTCTACCTCCTGAG
CTATCTTGCCTGCCCCTAATCATTTTTGACTCATGTATAGACTAATGGACATTTATTCAGTTGCATCATTTAGACGGTGGCTCAGGCTGTGACA
GTGTGGCACTCCTTCAGGTTGACATGTGTGGGCTCCTCTCACTGTCTTTGAGGCACCCTGTGTTTCTTGTTTTGGAGTTGTGTGAAGTTCATCT
TAGGGTCTTTCTTCCCTGGTCCTTCTGTCAGCCATTTGTCCTAGGCGCCTTGGCGCCTTATGTTGGGAATGCTGTTTAAAGGCGGTGCTGAGGC
CCTAGGTGTGTTGCAGATGAACTTTGATGACTTTGGCTCGCTGGGAATTAACACTGGCTTTCTGAAACTCTGGTGATACCGGGAAATCACTGAC
CACCCTGAGCCAAATGTAGGCACTACTGGATGGGACCGCAGGGCCTTCCTTACGGATTAGCACCCACCCAGGAGTTGCCTTCTACAGATAGAGC
CTGCTTGCTAACCATCAATCCGTGCTGTGTGTCTCTTGAGTGCTCGTCAAATTAGCACCACTCCTTAAGTAGCTAAAAAAAAATCCCAGCAATT
TGAGACATTTGAGAAAGGAAAATCTCCATAGCTGTGCTCTGGGCAGATCGCAGGGGGAATCTGAGTCACTGCCCCTCCTCTGTCCATTTTCTTG
GGATTCTTGGACCCTTAGCTAGAATCCTATGAGAATTGATGTGGGTGGTACCCACCATTGGTCAGAGGCTCTGCCACAGGCACCACACTTTGTG
GTCAAGGGCAGCTTTTTGCTGGTTTTGTCTTTTGAGAGAAGGTCTCCTGTAGTCCAGTCTGGCCTTGAGTTTTTATGTGTCTGAGGCTGGCCTT
GAACTCCTGATCCTCCTGCCTTCATGGCCAGGTGTGTGCCACTCATGTTTTATTGAGACAGGTCTGATTATGTAGCCCAGGCTGACCTCCAAGT
TATGATCCTCTTACCTCTTTGGGAGTTGGCTCCCTCCTCCCTCCATCATGTGGGTCTGGGCGATTGAACTCAAGTTGTTAGGCTTGGTGGCAAG
TGCCAGTACCTTAAGCCATATTACTGGCCCTAAACTAATTTTTTTTGATGTATTTTAGTCTATTGCCAATTCTTGTCTTACCGATGCTAACATT
TCCCCATCTTTGAAGGCCTTGGGTGGAAACCTTATTTTAGCAATTAGATCTGTTGGCCCAATAATTCCTGCTAATCTCTGATAGCTTAAAGAGC
CGCTTCAGGTTTACTTTGTATGTTTGCTGCCTTGGATCTGTAACTGGCCATTTCCCAAGAAACAGCAAGGCCTCTGGGTTTAGAGCCATGGTCT
GAAAGCCGCAGATGAACCCTTAATGTCACTGGATAAGGCCAGGAATGGGCAACTATGTGTTGTCTTTTGGAGCTCTGAGTCCTGGTTTCTGTGG
TTTAGGCTGAGAAGCAGTTGGCTAGGAAGTCATCTCTTTTGTAAGCCTGTGATGTAGTTCTAATTGTACTTGACAGATTTGATCTTAAAAAAAT
TTTTTTTATCTGGGCAGTGGTAGGTGGCCTTTAATCCCAGTACTTGGGAGGCAAACAGATCTCTGAGTTCAAGGCTAGCATGGCCTATAGAAAG
AGTTCCAGGACAGTCAGGGCTGTTATACAGAGAAATCCTGTCTTGAAAAGCCAAAAAACAAAGATTTTTTTTTTTTAAATTCACTTGTGTGTAT
GTGGCCTATGTACACAGTGCCTCGGTGTGCTTGTGGGGGTCAAAGGAAAATCTCCGTGCTTCATTTTCTTAAAACAGGCTGGCTTCCTTCCTTC
CTTCCTTTAAAAATACTGCATTTATTTTTATTTTATGTGCATTGGNNNNNNNNNNNNNNNNNNNNAAGCATGTACACACCTGCGTGAAAGGGAT
GTTTTTAGACTGATCCTCACACCCTGTTCCCATCGTGCCCTGTTCCCATCCTAGCCCATCACTTAACCTGTTTTACACCAAAAGTAGTCTTTAG
GGTGTGGTTAGCTATTTCTTTTGTGCCATTTTAGGGTGGAGAGGGTGGGCGTGATGGAGCCAGTCATTCAGGACTTAATTCTTCCTTGTGTTGT
GGTGTGGTTTTCTTTCTTCTTCTTCTTTTTTTTTTTTAATTCCTTCTTTCGCCATTGCACCAGAGTATGAAATAGCTTCCGGGTTCTCCGGCTC
TGAGCTGGGCGGTGATTGTGGTCACACCCTGACAACACTAGGGATCTCAACTGACTCCTCTTGTAGCCTCACCACTATTTTTTAGCAGTTTAAT
GGGTACATTATAGAGTCTTCCATTTTGTGTGGAATTAGCTCCTCCCCTTCAAATGCTGTAATTAACATCACTTAAAATAAAACTTGAATAAAAT
ACTGAAACCTCATCCTCTTGTTGTTTTTGTTAAAATACGAAATGGCCAGGGCATTGGTTTGGATTTGTGCTGTGCTCCTTTGTGGTGTCTATCA
ATAAATCTAACCTGATGGTAGAAAAGAACACTTGGGAGGCTGGAGAGGTGACTCGGGGGTTTATAGTGTTTCCTGCGCCTGTAGACCTGAAGCG
CTCACAACTTCCTATAACTATGGGTTTGGGGTATCTGATTCCCTCTTCTGGTCTCCTTAGGTACCCACACACATGTGACGCACAAATAAAAATA
AATCTTAAAAGCACTTAGGGTTGATGTAGTGGCACATCTCAGTACCTTGGAGATTAGCACAATGAGATCAGTGAGGAGGAAGGTTGCGGGGTTC
AGGAGAGCCCACCCTTAGTTGAGGCACTGCTGGCATTGATGACTGTTGGGTGAGAGTCAGTTTTCTTTGGGGGTGTATGCAGCTGCCCCAGGAA
GCTAGGGCCGCTGGCTGGATCTAAGGAGAACTGCAGTCAGTCACAGGCAGTTTGATGAGGGTAGTGGGAACGAACGGAACCAGGGTCATCGGCA
AGAGCATCCAGCACTGTTGAACCACCACCGCCTCTTCCAATAATACATAATAATCTCACTGTTGTAGAAGGTGTGTCTGGTTGCTTTGCTCAGG
TTTGAATGAGAATGTCCCCTTAGGCTCTGGCTTTTGAGTACTTGGTCTCTTGTAGGTGCAGTGTGGTAAGGCTTAGGGGGTGTGGCCTTGCCAG
AGGAAGTACGTCACTGGTGGGTGGGCTCTGAAGTTTCGCAGCTTATGCTGCTCCTAGTTTGTACTCTCCAATTCTTGTTTGTGGTTTGATGCCT
AGGCTTTCAGTTTGTCATTCCTGGTGCCAAGCCTGTCCGCTGCCACGCTTCCCGGCCTTGATGGTGATGGGCTGCTATCCCTCTGGAATTGGAA
GCCAAGATGATTGCCTTGGTCACGGTGTTTTGTCACAGCAATAGGAAAGTAAACACAACTAGGCTGCACCTTCATTCAGCAAGAGACTTTGGGT
CCTTTTCCAAGCCCACGAGGTTATGGTAGAATCCATTTGTTGTGAAACTGAGTCCCTTCTTGCCGGCTTGTTTCCCGACGACCACGCAGTACCT
GGAGAGCCCCAGCATTTCTTACATTGTCTCCCGTCTGCGGTGTATGGAACAGAAAGACTCGCTGTCTAATCCTTACTAGGCTCCTGTTCTCTAG
GACCAACTCTGTCTCTATTACCGGCTCACGATTAGGCGAGGCTCCCCTTTATAAGGATAACTGCCATGAAAAGTAAGCTCACCACACTTATAAC
TCTCATTCGCAGGCTTTCCCTTGTGGGCCATGGGAGGGGGTCGTTTTGAAGTTTACTCTTGGTTTTTCAAGACAGGATTTTCCTGGGCAGTCCT
GGAACTTGCCCTGAAGACCAGGTTGGACTCAGAGATTCACCTGCCTCTGCCTCTTAAGTGCTGAGGTTAAAGGTGTGCGCCACCACTGCCTGTC
TTTTTTCTTTTCTTTTTTTTTTTTTTTTTTTTTTTGGTTTTTCGAGACAGGGTTTCTCTGTATAGCCCTGGCTGTCCTGCAACTCACTCTGTAG
ACCAGGGTGGCCTCGAACTCAGAAATCCACCTGCCTCTGCCTCCCGAGTGCTGGGATTAAAGGTGTGCGCCACCACGCCCGGCAAAAATTTGTA
ACGTCTTTTTTCTTTCCTTTTTTTTTTTTAGATTTATTTATTTATTATATGTAAGTACACTGTAGCTGTCTTCAGACACTCCAGAAGAGGGAGT
CAGATCTCATTACAGATGGTTGTGAGCCATCATGTGGTTGCTGGGATTTGAACTCCGGACCTTCGGAGGAGCAGTCGGGTGCTCTTCCCACTGA
GCCATCTCACCAGCCCCCCACCCTTTTTAAAAAAAAATATTTATTTATTTGATTTATTTTTATTCATAGCTGTCTTCAGACAGACAGATCCGTA
GAGGGCATCAGATCCCATTACAGATGGTGGTGAGCCATCATGTGGTTGCTGGGAATTGAACTCAGGACCTCTGGAAGAGTAGTCAGTGCTCTTA
ACCACTGAGCCATCTCTCCAGCCTGCTTTTTTTTCTTTTTTTGAAGAAAGGTTTAATTGCTTCTATTTCACAGGTATGGATGTTTTGCCTGCAT
GTGTTTGTGAACCACATGCAGTCTGGGGCCTCCAGAGACCAGAAGAGGGAGTCAGATCCCCTGTGACTGGAGTTACAGATGCTTGTGAGCTGCC
ATGTAGAGCTGGGAACTGAACCTGGGACCTCTGGAAGAGCAGCCAGTGTCCTTAACCACTGAACCATCTCTGCAGCCCCCCTCTACTATTAACA
TTTTTTATGTTGTTTCTCCTTGTGTGTGCATGTGTGTTACAGAGAACAAGCGAGGGAGTCTCTTTCTTCCTAGTTTGTGGGTCCTGGGGATTGA
ACTCAGGTTGTTAGGCTCGGTGGCAAATGTGTTTACCTGTTGGGCTACCTCGAGATTTTTACTAAATCGTGAGTTCCTCCACGGAATAGGAATT
CTACCCGCACACTCTTCAGCTTCTATAGGAAGTGAAGTGGAGACTTCTGGTTTCTTTGGAAAGTCAGTTATGTCAGCGTTTTGCTGGGGCACCC
AGGTGAAAGGATGGTTTGCTGAAGCAGATACAGGTGGAAGGATGTTCTGCTATAGCAGACATGTGAAAGGACACATGAAGTTTAGAAGGAACAT
AAATCTGACCCCACGGACAGTGGGAGCTGGAGCATTGGTTTGCCTTGCTCTCCCTTGCTAGTTTTTGCTGACATCATGGATGCATTGGTTTGCC
TTACACTGCATTGCTGTGCTTTGCATGCAGTGACTTCATAGAGAGAGGCTTGCAAAAGAAAGGTCCCTGCGGCTTCTTGAGGCTTCCGTGGCAT
GGGGCCAGTTGGTGAGCCTCACAGTTCCTTCTGGATTCAACTGCAGTTGCTGATTCCTGTGTGGTATCTGCTGAGTGGACTGGACTGAGAACAA
CAGAGACTGGAATCTCCCCAAAGAGCTATGACTAAACATAGCTTATCCTAATAGCCTTCCTTTTCCACTATCTCTGCTGGTTGGTGGGCTAAGA
GGGGAGGTTGAACTCTTATTAAAGGAGGTTGAGAAAAATATATGCCTACAAGGAACATTCTTTTTTCTTGGCAGGATTCCTTTAAAAGAAAGAA
TTTTATTACTTTCTTTACTCTGTCTGGACATGTGTGGGCATACATTTGGAGTTCAGAGGACAGTTATTGGAACCATTTTCCCCCTATCTTTTTG
TGAGTTCTATGGATGAAACTTAAGTTCAAGGTTGTGGCAAGTATCATCTGCTCTTGTGTGAGAGTTGAGGCTTGTTGGTCACATCGACAGCACT
AGGTATGTATAGACGTGCAGCTAATTCTTGAGTGAGTGCTTAGGCTCTGACCAGAGACTTTAAAGAAAGTGGTTTCCCGGGGCTGGAGAGATGG
CTCAGTGGTTAAGAGCAGTGACTGCTCTTCCAAAGGTCCCGAGTTCAAATCCCAGCAACCACATGGTGGTTCACAACCATCCTTAATGAAATCT
GATGCCCTCTTCTGGAGTATCTGAGGACAGCTACAGTGTACTTACATATAATAAATAAATAAATCTTAAAAAAAAAAAAAAGAAAGAAAGTGGT
TTCCCTGTCCATGTAGTTGAAGGCCAGGCATAGTGTAAAGTGTTGTGAAATGTATAAAAGACCCCCCTAAGTATGATGGGATGTCATTCACCAG
GCACTTATTACATCATGCCTGCACACAGTCTGCAGTGTGTTAGATGGAATTTCACTGCAAAATGCCTTGAGTTCATAGTATCTCATTACTGAAA
AGAACTCAGATGGGGCTGGATGCTTCAGCAGGTAGAGCACAAGATGTTCTTGCAGAGGACCTGGGATTGGTTCCCAACATCCACGAGGCTGCTC
ACAACTGTCTGCAACTCCCGTTCCAGGGCATCTGATGCCCGTTTCTAGTCTCTGATAACACTACTCCCATGCCTAAGCCTCGAAAGATGAGACA
TATACACAGACATGAAAAATAATCTGAAAAATGGACTCAGCTGATCCATCACTGAGGACAGAAGAACAAAGTAGATTAGGGAGGATGATAGAGG
GTGTTGCAGAAGCCGATCAATTGAAAACCATCTCCAAAGAGGCAGAAGCTGGAATTGAGATTAGTCTTTGTAATGGTTTGTATAGGCTCAGCCC
AGGGAGTGGCACTATTGGAAGGTGTGGCCCTGTTGGAGTAGGTGTGGCCTTGTTGGAGTAGGTGTGTCACTGTGGGTGTGGGCTTTAAGACCCT
CATCCTAGGTGCCTGGAAGCCAGTATTCTGCTAACAGCTTTCAGATGAAGATGTAGAACTCTCAGCAACTCCTGCACCATGCCTGCCTGGATGC
TGCTTTGATGATAATGGACTAAACATCTGAACCTATAAGCCAGCACCAATTAAATGTTGTCCTTATAAAAGTTGCCTTGGTTCGAAACACACAC
ACACACACACACACACACACACACACACACACACACACACACACAAAGAGTGGCCTTGGTCATGGTGTCTGTTCAAAGCAGTAAAACCCTAACT
AAAATAGTCTCTAAACACTGGTATTTTCTAGACTGGCAGAAAGCAAAGCTTTGGGGTGCTTGGAGGGACTGTACCTCTGCTAATCTGGAAGGTT
TGGGCACCATTCCATGTACATGGGCTTTATGGCAGACTGTGAAATGAGGTAGATGAATGGCTTCTTGGTTTGTTTAACAGAATCTGTGTGCTTT
TAAGATGTCTTGGGGTCAGAGAAATTGGCTTTGGGAAAACACAAGCTGTTTCTGGGGAGGGAATTGCAGAGACAGCAGTGAAGCTGTTGGGGGC
ACAATAAATGCCAACTGCTCATTTTACTGAAAATTAGTGAAATGAACATATATGAATTGAAATCTATAAGAAACATTCACACAACATACTCACA
GATGGAAACTGTACCAAGGCTCTAGGCAAACTTTCTAAATTCACACTTCTCTTCTTTCTTTTTCTTTCTTTCTTTCTTTCTTTCTTTCTTTCTT
TCTTTCTTTCTTTCTTTCTTTCTTTCTTTCTTTTTTCCTGTGTGTGTGGGCAGGGAAAGGGGGAGAAAGCATTTCTCTGTGTACCCCTGGCTGT
CCTGGCACTCACTTTGTAGACCAGGCTGCCCTTGAACTCAGATATCCACCTGCCTCTGCCTCCCAAGGTTTATATTTTAGTGTTTCATCAAATA
TTTAACACTTCTCCGTAGTTAAGGGTGCATCAATAAACAAAGTAAGGTCAAGTTGGCCTTGGCAGGCTTGCTTTTGTGTGTGGGGCGCTGTAAG
AGAGGATAATATTTAACCGGATGATAGTGTTATAAAAGAAAAGCAAGGAAATTGGCTAGAAAGTCAGAAGGAGACACTGGCATTGAGAATAGGT
TCAAATGTTCCAAAGACATACGTCTATCACTTTTTACATTTTGTAAAACAAACCTCTCAGTATACAAAAATATAACCCAGCAAGTGGGAGGTGA
AGGTGAAGGAGTTCAAGGTTATCCACTGGGTAGAAAGGGATTTTGAATGCCGGTGCCTGGCAAACACAGAAGTGGATGCTCACAGTCAGCTATT
GGATGGAACACAGGGCCCCCAAAGGAAGAGCTAGAGAAAGTACCCAAGGAGCTAAAGGGGTCTGCAACCCTATAGGTAGAACAACAATATGAAC
TAACCAGTACCCCCGGAGCTTGTGTCTCTAGCTGCATATGTAGCATAAGATGGCCTAGTCGGCTCGGCCATCATTGGGAAGAGAGGCCCCTTGG
TCTTGCAAACTTTATATCCCCCAGTACAGGGGGAACGCCAGGGCCAAGAAGTGGGAGTGGGTGGGTAGGGGAGTGGGGGGGGGGGGATATGGGG
ACTTTTGGGATAGCATTTGAAATGTAAATGAAGAAAATACCTAATTAAAAAATAAAATTAAAAAAAAAAGGAATTTTGAGGCCAGCCTGGGCCA
CTTGAGATCTTGTTTCAAAACCAAAACCAAAAGGGTGCTGGAGAGAAATTATTCAACAGTTTAGAGCAATTGTGCTTGTAGAGGACCAGGGTGG
TTCCCAGGAGCCACATGGGGTGGCTCATAATCACAGCAACCTCCAGCTCCAGGGGAGTAGACGCCCTCTTCTGGCACTGGAGGGCATTTGAACT
TACACACAATTTTACTCTTTCTTTATTGTTTCTTTAATGCTTATTTTATCATTATTATTTTTTGAGACAGGGTTTCTCTGTGTAGCCCTGGCTG
TTCTATAATCGCTTTGTAGACCAGGCTGGACTCGAATTCACTTAGATTTACCTGCTTCTGCGTCCTGAGTGCTGAAACTAAAGATATGCACTAT
CACACCTGGCTAGTTTTAATTCAGAAATGATTGCTTGCATATATGTTTGTGTACCCCATATGCCTAATGCCTGTGGAGATCAAAAGAGGTCTTA
TAGTCAGATCCCCTGGAACTGGAGTTAGAGGTGGCTGTGAGCTGTTTAATCTGAGTGCTGGGAACCAAACCCAGTCCTTTGCAAGAGTAGCAAA
TGCTCTCAACAGCTGAACATTCTCTCCAGCTCCATATATATATATATAATCTTTCTTAAGTGTATTAAACTTGTGTGTAGTTGTTTTACTTATG
AGTTTCTGCATCAGTTATACGTCTAGTGTTTGAGGATGCCTAAAGAGGGCACCGTATACCCTGGGACAGATGGGTGTGAGTCCTCACATACAGG
GGTGCTTGGAATCGAACCCAGGAGCTCTGGGGGAATCAGCCAGTGCTCTTAAGCGATGACCCCTGAAATACATCTCAAAGATGACAACGATAAA
CCACAGAGCAAAACAAAAAACCACCAGTTGAAAATAAATCCGTCCCATTCGCCTTTTTTTTTTTTTTTTTTTTTAAGCACCCTTTTCTACACTC
CCACGCTAACCTTTCGGGTTCAGAGATGCAACGTCATCACGCCGGGACGTAGCTGCTT
MOUSE SEQUENCE—mRNA
CCGTAGCTGCCGCTGCTCCCGCCGTCACCGCCTCCGAGAACATGGCCGTGCCTCCCACATACGCCGATCTTGGCAAGTCCGCCAGGGATGTCTT
CACCAAGGGCTACGGCTTTGGCTTAATAAAACTTGATTTGAAAACGAAGTCAGAGAATGGATTGGAATTTACCAGCTCAGGCTCTGCCAACACG
GAAACCACCAAAGTGAACGGCAGCCTGGAAACCAAGTACAGATGGACTGAGTATGGGCTGACGTTTACAGAGAAGTGGAACACAGACAACACCC
TGGGCACTGAGATCACTGTGGAAGACCAGCTTGCTCGTGGACTGAAGCTCACCTTTGATTCGTCATTCTCGCCGAACACTGGGAAAAAAAATGC
TAAAATCAAGACAGGGTACAAGAGGGAGCACATCAACCTCGGCTGTGACGTGGACTTTGACATCGCTGGGCCCTCGATCCGGGGCGCTCTGGTG
CTTGGCTATGAGGGTTGGCTGGCTGGCTACCAGATGAATTTTGAGACCTCGAAGTCCCGAGTGACCCAGAGCAACTTCGCAGTTGGCTATAAGA
CGGATGAATTCCAGCTTCATACTAATGTGAATGACGGGACAGAGTTTGGTGGCTCCATTTACCAGAAGGTGAACAAGAAGTTGGAGACTGCTGT
CAATCTCGCCTGGACTGCAGGAAACAGTAACACTCGCTTCGGAATAGCAGCCAAGTATCAGGTCGACCCTGATGCCTGCTTTTCGGCCAAGTGG
AACAACTCTAGCCTGATTGGCTTAGGGTACACTCAGACCCTAAAACCAGGTATCAAACTGACGTTGTCAGCCCTGCTCGATGGCAAGAACGTCA
ATGCGGGTGGCCACAAGCTTGGCCTAGGACTGGAATTTCAAGCATAAATGAATATTGTACAATCGTTTAATTTTAAACTATTTTGCAGCATAGC
TACCTTCAGAATTTAGTGTACCTTTTAATGTTGTATGTTGGGGATGCGAGAGTTGATAAATACCACGTTAGACCTCCAGGCTAAGGATGACTCG
GCTTTAAGGTGTTTACCATTTCAGAGGTACAGCAGAAACCCCATTCCAGAAAGGGTCCTTTTTAGCTGTAGGCGTGGGTTGGGGAGGAGCCCCT
GTAGAGATGCCAGGCTACAAGTGGAAAGCTGGGAACATGTGGGTCCTTTGTAAATCTGTATCCAGTCCCCAGATGAAATTGTGACTTCCCGAGC
ATCGAACCCTGGTGTCCAGATCCTATCTGCTCGGAAGCATGTACACACCTGCGTGAAAGGGATGTTTTTAGACTGATCCTCACACCCTGTTCCC
ATCGTGCCCTGTTCCCATCCTAGCCCATCACTTAACCTGTTTTACACCAAAAGTAATCTTTAGGGTGTGGTTAGCTATTTCTTTTGTGCCATTT
TAGGGTGGAGAGGGTGGGCGTGATGGAGCCAGTCATTCAGGAATTAATTCTTCCTTGTGTTGTGGGGTGGTTTTCTTTCTCCTCCTCCTCCTTT
TTTTTTTAATTCCTTCTTTCGCCATTGCACCAGAGTATGAAATAGCTTCCCGTTCTCCGGCTCTOAGCTGGGCGGTGATTGTGGTCACACCCTG
ACAACACTAGGGATCTCAACTGACTCCTTTTGTAGCCTCACCACTATTTTTTAGCAGTTTAATGGGTACATTATAGAGTTTTCCATTTTGTGTG
GAATTAGCTCCTCCCCTTCAAATGCTGTAATTAACATCACTTAAAATAAAACTTGAATAAAATACTGAAACCTCC
MOUSE SEQUENCE—CODING
ATGGCCGTGCCTCCCACATACGCCGATCTTGGCAAGTCCGCCAGGGATGTCTTCACCAAGGGCTACGGCTTTGGCTTAATAAAACTTGATTTGA
AAACGAAGTCAGAGAATGGATTGGAATTTACCAGCTCAGGCTCTGCCAACACGGAAACCACCAAAGTGAACGGCAGCCTGGAAACCAAGTACAG
ATGGACTGAGTATGGGCTGACGTTTACAGAGAAGTGGAACACAGACAACACCCTGGGCACTGAGATCACTGTGGAAGACCAGCTTGCTCGTGGA
CTGAAGCTCACCTTTGATTCGTCATTCTCGCCGAACACTGGGAAAAAAAATGCTAAAATCAAGACAGGGTACAAGAGGGAGCACATCAACCTCG
GCTGTGACGTGGACTTTGACATCGCTGGGCCCTCGATCCGGGGCGCTCTGGTGCTTGGCTATGAGGGTTGGCTGGCTGGCTACCAGATGAATTT
TGAGACCTCGAAGTCCCGAGTGACCCAGAGCAACTTCGCAGTTGGCTATAAGACGGATGAATTCCAGCTTCATACTAATGTGAATGACGGGACA
GAGTTTGGTGGCTCCATTTACCAGAAGGTGAACAAGAAGTTGGAGACTGCTGTCAATCTCGCCTGGACTGCAGGAAACAGTAACACTCGCTTCG
GAATAGCAGCCAAGTATCAGGTCGACCCTGATGCCTGCTTTTCGGCCAAAGTGAACAACTCTAGCCTGATTGGCTTAGGGTACACTCAGACCCT
AAAACCAGGTATCAAACTGACGTTGTCAGCCCTGCTCGATGGCAAGAACGTCAATGCGGGTGGCCACAAGCTTGGCCTAGGACTGGAATTTCAA
GCATAA
HUMAN SEQUENCE—GENOMIC
TATATTGCATGGTGCTGAGGTTTGGGGTATGATTGAACCTGTCATCCAGGTATTGAGCATAGTACCCAACAGGTAGTTTTTCAACCCTTGCCCC
CCTCTCTCCCTCCCTCCCCTCTCTTGTAGTCCACAGTGTCTATTGTTCCCATATTTATGTCCATGTGTATCCAATGTTTAGCTCCCACTTATAA
GTGAGAACATGCAGTATTTGGTTTTCTATTCCTGCATTCATTCACTCAGAATAATGGCCTCCAGCTGCATCCATGTTGCTGCAAAGGACATGAT
TTCATTATTTTTTATGACTGTGTAGTATTCCGCCATATATATGTACCATATTTTCTTTATTCCATCCACCACTGATGGGCACCTAGGTTGACTC
CATGTCTCTGCTATTGTGAATCACGCTGTGACAAACCTACATGTGCATTTGTCTTTTTGGTAGAATGATTTATTTTCTTTTTTTGAGATGAAGT
CTCACTCTGTTGCCCAAGTTGGAGTGCAGTGGCACGATCTTGGCTCGCTGAAACCTCCACCTCCCAGGTTCAAGCGATTCTCCTGCCTCAGCCT
CCTGAGCAGCTGAGACTGCAGGCGTGTACCACCATGCCCGGCTAATTTTTGTATTTTTAGTAGAGACGGGGTTTCACTATGTTGGCTAGGCTGG
TCTTGAACTCCTGACCTTGTGATCCGCCTGCCTCAGCCTCCCAAAGTGTTGGGATTACAGGTGTGAGCCACCGCACTCAGCCATGACTTATTTT
CCTTTGGGTGGAATGGTAGTTCTATTTTTAGTTCTTTGAGAAATATCCAAACTGCTCTCTACACTGGCTGAACTAATTTACATTCCCACCAGCA
GTGTATAAGCATCCTCTTTTCTCTGCAGCCTTTCCAACAACTATTTTTTAACTTTTTAATAATACCCATTCTGACCGGCGTGAGGTGGCACTCA
TTGTGGTTTTGATTTGCATTTATCTGATGATTAGTGATGATGAGCATTTTTTCATGTTTGTTGACCACTTGTATGTCTTTTTATTTTTATTTTT
TTGAGACGGAGTCTCACTCTGTCATCCAGGCTGGAGTGTGGTGGCACGATCTCGACTCACTGCAACCTCCACCGCCCCGGATTCAAGTGATTCT
CCTGCCTCAGCTTCCTGAGTAGCTGGGATTACAAGCGCGTGCCACCACACCTGGGTAATTTTTGTATTTTTAGTAGACTCGGGGTTTCACCATG
TTGGCCAGGTTGTCTCAAACTCCTGACCTCAGGTGATCCGCCCACCTTGGCCCCCCAAAGTGCTGGGATTACAGGCGTGAGCCACCATGCCTGG
CTGTATGTCTTCTTTTGAGAAGTGCCTGTTCATGTCCTCTGCCCACTTTTTTTCGGGGGGTGGGATGGGAGACGGAGTCTCACTCTGTCACCCA
GGCTGGAGTGCAGTGGCACGATCTCAGCTCACTGCAACCTCCGCCTCCCGGATTCAAGTGATTCTCCTGCCTCAGCCTCCCGAGTAGCTGGGAT
TACAGGTGCCCGCAACCATGCCTGGCTAATTTTTGTATTTTAGTAGAGATGGGGTTTCACCATGTTGGCCAGGATGGTCTTGAACTCCTGACCT
CAGGTGATCTGCCTGCCTTGGCCTCCTAAAGTGCTGGGATTACAGGTGTGAGCCACCGTGCCCAGCCCTCTGCCCACTTTTTAATGGGGGTCTT
TGTTTTTTGCTTGTTGAATTGTTTACATTCCTTATAGATTCTGGATATTAGAGCTTTGTTAGATGCGTAGTTTGTAAATATTTTCTCCCATTCT
GCAGGCTGTCTGTTTACTCTGTTGATAGTTTCTTTTGCTGTGCAGAAGCTCTTAAGTTTAATTAGGTCCCACTTGTCAATTTTGTTTGTTTGTT
TGTTTGTTTTTGAGACACAGTCTTACTCTGTCACCAAGGCTGGAGTGCAGAGGTGCGATCATAGCTCACTGCAGCCTCGACCTCCTGGACTCAA
GTGATCCTCCCACCTCAGTTTCCTGAGTAGCTGAGACTACAGGTGTGCACCACCATGCCAGGCTAATTTTTGTATTCTTTGTACAGACGGGTTT
TTGCCATGTTGTTCAGGCTGATTTTTGTTTTGCTGTAACTGCTTTGGAGGACTTAGTTATTTATAAATTCTTTGCCGCGGTTGATGCCCAGGAG
GGTATTTCCTAGGTTTTCTTCTAGGATTTTAATTGTTTGAAGTCTTACATTTAAGTCTTTAATCCGTCTTGAGTTAATTTTTGTATATGGTGAT
AGGCAGGGGTCCAGTTTCATTCTTCTGCATATGGATAGCCAGTTATTCCAGCACCATTTATTAAATAGGGAGTCCTTTCCCCATTGCTTATTTT
TGTCCAACTTGTTGAAGATTAGTTGGTTGTAGGAGGGTGGCTTTATTTCTGTGTTCTCTATTCTGTTCCATTGGTCTATGTGTCTGTTTTTGTA
CCAGTACTATGCTGTTTGGGTTATTGTAGCCTTTTAGTATAGTTGGAAGTCAGGTAATGTGATGCCTCCAATTCTGTTATTTTTGCTTAGGATC
CTTTCGTTATTCTGGCTCTTTTTTTTGTTTAATATAAATTTTATAATAGTTTTTTTTCTAATTCTGTGAAAAATGATGTTGTTAGTTTGATAGG
AACAGGATTAAATCTGCAGATTGCTTTTGGCAGTATGGACATTTTAATGATATTGATTCTTCCAATCCATGAGCATGGAGTGTTTTTCCATTTG
CTTGTGTCACATAGATGATGCTTTCTAACTGTGTCTTTACATGGTGGAAAGGGTGAACAAGCTCTCAGGCCTCTGCCCTAATGATAATTACATT
TCAAAAGCTCTGCCTTCTAATACCGTCATCTTGGGACTCAGGATTTCAACATATCAATTTTGGGAGTAAACATTCATACTATAGCCCCATTCTA
ATAAATCATTTCTAGAATTGTCTTTTAGAGGTTGTATATTAGTTTCCTATTGCTGTTGTAACACTTCCTATTGTGTTGTAACTGTGAACCACAG
ACTCAGTGGTTTAAACAACTCAGATTTATCATTTTACAGTTCTAGAGGTCAGGAGTCTGAAATCAGTCTCTCTGGGCTAAAATTAAGTGGGCAT
CAGGGCTCCCTCCTTCTGGAGGCCTTGGGAGGAGAATCTGTTTCCTTGTCTTTTTCAGCTTCTATAGGTTGCCTGCATTCCTTGGCTCATGGCT
CCTTCTTACATCTTCAGAGCTAGCGGTGTAGCATCTCCAATCTCTCTCTTTCTTTCTTCTTTTTTTCTTTCTTTCTCTTTTTCTTTCTTTCTCT
CTCTGTCTCTCTCCCCACCCCTGCCCTGCTCTGCTTCTGTCATTAAATCACCTTCTCTCACTCTGACCCTCTTGTCTTCCTCTTATAAGAACTG
TAGGCCGGGCATGGTGGCTCATGCCTGTAATCCCAGCACTTTGGGAGGCTGAAGTGGGTGGATCATCTGAGGTCAGGAGTTTGAGACCAGCCTG
GCCAACATGGTGAAACCTCGTCTCTACTAAAAATACAGAAATTAGCCGGGCCTGGTGGTGGGCACCTGTAATCCCAGCTACTTGGGAGGCTGAT
GCATAAGAATTGCTGGAACCCAGGAGGCGGAGGTTGCAGTGAGCTGAGATCACGCCACTGCACTCCAGCCAGGGTGACAGAGTAAAACTCTGTC
TAAAAAAAAAAAAAAAAAAAGAACTTGTGATGACATTCTTGGGATTACATTGGGCACACTTGGATGATCTAGGACAATCCCCCCATGTCAAGAT
CCGTAACATCTGTATGATCTCACTTAAATGTACAATCTAGAATAATCACACTCATAGAAGCAGAGAGTACAATGGTAATCTTCAGGGCTGGAGG
GAAGGGAAAATGGAGAGGTGATGATCAAAGGGTATAAGGTTTAGTTACGCCAGATAAATAAATTCTGGAGATCTATGCATCTGACAAAAGGTAT
AATATCTAGAATCTATAAGGAACTTAAAGAGTTCAACAAGCAAAAAAACAAATAACCCCATTAAAAAGAGGGCAGAGGGGGCTGGGCGCAGTGG
CTCACACCTGTAATCCTAGCACTTTGGGAGGCTGAGGCAGGCAGACTGCCTGAGCTCAGAAGTTTGAGACCAGCCTGGGCAACATGGCAAAATC
CCATCTCTACTAAAAACAAAGAAAAAAATGTGGGCAGAGGACATGAACAGGTGCTTCTCAAAAGAAGACATACAAGTGGCCAAGAAACATATTA
AAAAATGTTCATCATCACTAATCATCATCTACTATACATCACAGTGCCTACAGCTAACAATACTGCACTGTATACTTAGATTTGCCAAGAGGAT
AGATCTTAAGTGCCCCTACAAAAAAAAAAAAAGGGAATGGGGGTAGGGGAGGAAACTTTGGGAGGTGTTGGGTATGTTTATATGCTTTACGGTG
GTGATGGTTTCATGGAAATATACTTATCTCCAAACTCATCAACATATATATATTAAATATGTACAGCTTTTTGTATGCAATCCTATCTCAATGA
AGTAGTTTTTGTTTTGTTTTGTTTGTTTTTTACATTCACAGGTTCCAGTGAGTAGGATGTGGATATCCTTGGGGGGCCATTGTTGTACTTACCA
TAGTTTGCCTCCACAGTTTTTAATGTTGGGGTTGCAGTAATACTGAACATAGATTGCCTTCCATTAACATTCTAACAGAAATATTTCCCCACAG
GAAATTTGTTGTTGTTGTTGTTGTTTTGAGACAAAGTCTCGCTCTTGTCCCCCAGGCTGGAGTGCAATGGTGTGATCTCGGCTCACTGCAACCT
CTGCATCTCGGGTTCAAGCCATTCTCCTGCTTCAGCCTCCCGAGTAGCTGGGATTACAGGCGCCTACCACTATGCCTGGCTAATTTTTGTATTT
TTAGTAGAGGCAGGGTTTCATCATGTTGACCAGGCTGGTCTCAAACTCCTGATCTCAGGTGATCTGCCCGCCTCAGCCTCCCAAAGTGCTGGGA
TTACAGGTGTGAGCCACCACGCCCGGACCCCCACAGGAAGTTTTTGTAACCATATTTCCTCAAGGTTGTGAGTGATTCAATATGCCCAAGCTGG
AAACCAGGTTGTGTTCCGTCCTGGGCTGCTGTCCATTTTCCTGCAGCAGGCATTCCCCATACTTACAATGATGGTCTTAGCGTGGGTTCCCAGC
ATCAAGGGGGGTGGAATAGGATTATGATTCTGATCGCATATAGCCTAATTGCTTTTCAGAAGGATTGTATCACTTTACACATCTCCTGATGAAC
CTCATATGACTTTCTTAAAGCTGAAAAAAGTTCGCTCTTTTTATAATCAAGGTATTATGTCCTCATTAAAGCATATTTGGAGGCTGGGCCTGGT
GGCTCATGCCTGTAATCCCAGCACTTTGGGAGGCCAAGACAGGTTGATTACTTCAGGTTAGGAGTTTGAGACCAGCCTGGCCAACGTGGCAAAA
CCACATCTCTACTAAAAATACAAAAATTAGCCAGACGTGGTGGCACGCACCTGTAGTCACAGCTACTTGGGAGGCTGAGGCATCAGAATAGCTT
AACTCGGGAGGTGGAGGTTGCAGTGACCCGAGATGGTCCCACTGAACTCCAGCCTGGGTGACAGAGGGAGACTCTGTCTCAAAAAACAAAACAA
AACAAAACAAACAAACAAAAGAATATTTGGGAAATGTAAAAAGAGCCAAAGGAAAAAATCACTCATAGTCTCATAAATAGAAACACAACTTAAT
AATTATGTTTCAATTTATAAGAATTGATCAATTCTGGCTGGGTGCAGTGGCTCACACCTGTAATCCCAGCACTTTGGGAGGCGGAGGCCGGCAG
ATCACAAGGTCAGGATTTTGAGACCAGCCTGGCCAACATGGTGAAACCCCATCTCTACTAAAATACAAAAATTAGCTGGGCCATGGTGTCGTGT
ACCTGTAATCCCAGCTACTTGGGAGGCTGAGGCAGAATTGCTTGAACCAGGACCTGGGAGGCGGAGGTTGCAGTGAGCCAAGATCATGCCACCG
TACTCCAGCCTGGGCTACAGAGAGAGACTCCATCTAAAAAAAAAAAAAAAAAAAAAAAAAAAGAATTGATAAATTCCCATAAATACTTCATTAA
TACTTAATACATTTCTTTCCAATATTTGTCTCTCTGTGTATATATATACAAAATTCTTAAAAATCATAGCTATGATGTTGGAGCTTCTTGGAAT
TGTTTTAGATTAGATCTAGGACACCTCATGTTTTGGGATATGCAGGAGAATATTATCAATTGTGTTTACCTTGGGATGCTTTCTGTCCTGTTTC
CCCAAGAGATTACTGCTGTCACTCAGAACCTGGAGGCCCAGCTGGGTCCTTCCCTGGATGCTGAGTGTCAGGGCACAGTGTGGGGAGTGTGGCC
TGAGATCCTTGACCACAAACACGCCATCTTGATTCTTCCTACCCAGACTCTAGTCTGAGGAGTTCACTGGAAAATGATTGGAAAGTTAGGTTTT
ATGACAATGCAACTTTATAATCCAATTAATTGATGCTGGTTACAAGCAGAATATCTTTCTCTTTAAAAATTAATTTACATAAATGACTACCATT
TCTGCCTTCATGAACTTCATCTTCAAGCTCTGGTACTTAGCACAGACAATGCTAAGAAACCACATAGAAATCCAGGGACATCTGGCTCTGAAAA
GAGAAGAGTTGGGAGTGTTTCATCCTATGTTCTTGAAAGGGGGGTGGAATAGGTTTATGATTCTGATCATAAATTGGCTGCTTTCCACTTTGAT
AAATATCTACAGTAGAAGCCCTAAGGCTGTAAGTTAGAGATAAGACAAAAACCAAAAACTCCCTAAAATGAAATAGTCTTACATTGGTAACCCA
TTCCAATATTAACAATGAAAGAAAAATCTCATTTTCCCTGGATGTTTTACATTGTTCTTCTTGAATGGAGGAGAGGAACTGAGTTAGCATCTGG
AAGAAAACAATCCAAACTCGACTAGGCAAAACAGGAGGCCGTTCGCTCAGGCAGCTGGTAAGTACAGGGTGTAGTCTCCGGCATGGCTAGATTC
AGGACCTCATCTCTGCCTTTCTCTTATGGGCTTACTTCATTTTCAGGGAGTCTTTATTCCTGTAGCCCCAGGAATTCTCATCTGGTCTCATTGG
TCCATCCCTGCGGGGCGGGGATCGGGGGCGGTGGGAGGCCAGGATGTGGGGGCGGAGAGGCGGGGGCAGGAGAGGTATACAGGGCTCTAATTGG
TCAGACTGGGCCATGTGGCCACTCCCAGGAAGGCTTTGGAGTCAGGGATGGAGTCAGCTCCATGAAAGCTTGCTGACCAAAAGGAGAGGGAAAC
AGGGTCATTCGCCCAGAGAAAAACTGAGGTTAGGATTGTCCTGTCCCCACAAACCTCCTTCTCCACCCACCAAGATTTGACTGTCTTCGCCAGG
GACTTGCTCTAGCTGGGTCAAGCTCAAGTGGGGTTATTGAAGTGGAACAGGGAAACCCACGCATGGCAAGAACACGGGGGCACTCAAGGAACAG
GGGCCCAGAGCCACCCTCTGCAGCGGCTGCTCCATTCTCTGTTCTGTGAAAAGCCCTCGTTAACTTCGCAACACCGACAGGGATGCCAGACCCA
GGTCTCCCCAAGGGCCTTGAAAGCTCCCGCATTTTCCCCTCTGCCATACTCATCCCTGGGTTTCTCAGTTCAGGTTTGCAAGAGAGGATCTGAT
TGACCTGGCTTGGGTCAGGGGCCCAACCCTGACCCAATCAGCTGTGGCAGATGGAATGGAGGTGCGGCTCAGGGGGTTTCCCCTCTCCCTCAGG
GTTGTGGTGGGTCAATGGATTCTAGTGATTATGGCACCTGCAGATCTCAGCTGATTTTGTTTTTTAAAAAATTACTAATACTATTTCTTGAGAC
GGGAACTTGCTCTGTGGTCCAGGCTGGAGTGCAGTAGTGGGATGCTAGCTCACTGCACCCTCGAACTCCTGGCCTCAAGCCATCCTACCTCCTT
GGCCTCCCAAACAGTTGGGATTACCCACATGTGCCTCTGAGCCTGGCCCAGTTACTGATGAGTGCCTACTCTTTTTTCTTTTTTTTTTTTTTTG
GCAGAGTTTCGCTCTTGTTGCCCAGACTGGAGTGTGATGGTGAGATCTTAGCTCACCGCAACCTCCGCCTCCTGGGTTCAAGCAATTCTCCTGC
CTTAGCTTCACGAGTAGCTGGGATTATAGGCATGTGCCACCATGCCCGGCTAATTTTTGTATTTTTAGTAGAGACGGGGTTTCTCCATGTTGGT
AAGGCTGGTCTCGAACTCCCGACCTCAGGTGATCCAGCCGCCTCAGCCTCCCAAAGTGCTGGGATTACAGGAGTGAGCCACTGCACGGGGCCGA
GTGCCTACTCTTTATGTGTCCCATTTTAAACTTACACCAACACTCTGAAGCTGAGGCTCTTAGAGGCCAGCCTTCTTGCCTGGGTCACGCAGCC
ACTAAGTGAGTGGTTAATCTGAAACTAGCACCTAGGGGAGCTGGCGAAGGGGCCCAGGGTAGCAGAGGCCATGAAGAACTCTTTCCAAGTTGTT
GTTTTTTTTTGTTTTTTTTTTTTGGGGGGGGGGGACAATGTCACTCTGTCAGTCAGGCTGGAGTACAGCGGCTCAATCTCAGCTGACTCCAGCC
TCCTCCTTCCAAGTTCAAGCGATTCTCGTGCCTCAGCCTCCTGAGTAGCTCGGACTACAGGCGCGCGCCACCACGCCCGTCTACTTTTTGTATT
TTTAGTAGACACAGGGTTTTGCCATGTTGCCCAGGCTGGTCTCGAAGTCCTGGCCTCAAGTGATCTGCCTGTCTCGGCCTCCCAAAGTACTAGG
ATTACAGGCTTGAGCCACTGCACCCGGCCCAGATTGGTTCTTTAAAGCGGAGTTGAGAGGTGGCGCTGGAGGGGAGGAAGCCCTTCGACATTTG
CTCCCAGCCTCAAAACTGGGTTGCACCGGCATCTGTTCCTAGCCCTGTGGGGTTCCTGGAGCCCCGCCTTGTCCCGCCCGGCCTCCCTGAGTTC
TTGTCCTCCCCCTGCCCACCAGTCTCAGTGTGCGGGGTGGGGACCACCAGGCTGGGAAGCCCAGAGTTCCCAGGCAACTCGTGCGCCTGGGAGG
GTCATAGAATCCGTCCTACACTCCTCAGGATCTTGAAGGGGGCTCTTCCTTACATTCCCAGTCACCCCTCCCCCAGCCCCTACACAGGGCCTAG
GTAACCTGTCTGCACCCCACTCCCACTCTGTATCAGCTGCCCTCTTCCTCAGCACCCTATCCCACTGGAGCACCTTTTCCAACTCCAGTCGCCA
CTTTTAATCTGCCGCCTTTTCCCAGCAGCCAGTCCTGATTGCCTCCTCCGTAGCCCCAGTTGCCCACTGCTTTAGATAGTGTCGCAGCTGGCTG
TGAGGAGTGTCCTGATTCTGTAACCCGGCACCTCCTCCACAGGGCCCTAGACGCCTCAAGCCTTCCAGGACGTTTGTTGATCAAAGGCGAAGGA
TGTGCAGAATCTTCGTCCATCTACGAATTAATTCTCGAAACCTGCTTCGAGTGGGCCTCGCGCGTAACACAGGGGACACTCAGTGGTGAACGAG
ACCACTCGCTTAGGCCTAGCAGCGAGGTGACCGTTCACTCAACACACACTAACTGGGTCCCGCAGGACACGCCCCGGCTGGTCGCGGGGGTTAC
CCGGGGTGCCCTCAGTCGGCCGCCCGGCCATAACCGGCGTGATCTCCTCGCGCCGAGAGACCCAGCAGGGACCTCGCAAAACCCAATCCACGTT
TTGTTTTATTTTGTCTTTTTGCCTCTCAAATGCATGGAAATTGGCTGCTAGTAGCTTTGCTACAGGAGAAAATCGCTAATCTGTAGTGCAAAAT
ACCACTCGCTTAGGCCTAGCAGCGAGGTGACCGTTCACTCAACACACACTAACTGGGTCCCGCAGGACACGCCCCGGCTGGTCGCGGGGGTTAC
CCGGGGTGCCCTCAGTCGGCCGGCCGGCCATAACCGGCGTGATCTCCTCGCGCCGAGAGACCCAGCAGGGACCTCGCAAAACCCAATCCACGTT
TTGTTTTATTTTGTCTTTTTGCCTCTCAAATGCATGGAAATTGGCTGCTAGTAGCTTTGCTACAGGAGAAAATCGCTAATCTGTAGTGCAAAAT
CGGCGCACAGGGGTTTTCTCTGAGATTGTCTAGACGCCGCGCGGGCGGGTCGGAGGCGCCAGACACATTGTATCCGGAGCGCGGAGCTCGGCAG
CAACAGTCGCCGCCTCTACTCTGTATCTGCGCCGCCCCAGGCGCCCACTGTCCCCGCTGGGCCTGGGACTGCCCTCGGGGGATGGGGCCCACGC
GGGCTTTGATGGGGGGCCGGGGGCCACTTTGTCCACCCAGGCGAGGCTTGGGTCCAGCGCCGGCCACATCAACTCCCGGGGGATCTAGCAGCGC
GGAGGCCAAGCAGGAGGCCCAGGAGCCCCGCGCGTCTTCCATTAGCGCAGGGACCTCCGGGCCACAGCTCAGAGAATCGGAAGGCCTCCTCCCC
CTTCCCGAGCGCTGCCACTGGGGCCGAGGTTTCCAGCAAGAACCCGCGTGTCCCTGCGCACGCACACACGGTGCACACGTCAGTCCGGCGCCTC
CCCGTGCCCCGACTCACGCAGGTCCTCCCGCGCGCCCGCAACACGCCCGCAGGCTCCTGTGTCTGCTGCCGGGGCAGCGGGGCCCGGAAGGGTA
AGAACGCGGCTCGCTAGCGGGATGGGGACCCTGGCCCGGCCTGGACGCTGTCCCGGGCGGGGCCTGGGGGCGGAGCGCGCCGGGCCGGGCGCGC
AGGAGGGGAGGCGGGTCCGCGCCGCCGCCGTCCGCGCCGCCCCGCCCTTGGCCCAGCCGCTCGCTCGGCTCCCCTCCCTCGCTCGGCTCCCTGC
CTCCGCGTCGCAGCCCCCGCCGTAGCCGCCTCCGAGCCCGCCGCCACATCCTCTGAGGTAGGCGCACCGGAAAAGGACCCCGGCTTCCTTGCGC
CCTGGACGGCGGGGGCCAGGCCCCTGGGGAGCTGAGAGGGAGGCGCTGAGGGAGGAGCGGGCGAGGGGCCCCGGCAGCCCGGCCGCGGGGAGGG
ATCGCGAGCGGCCGGCGCGGGCCTGGCTGGTGAGCGGGCGGGCAGAGAAGGCCGCACAGGCGGGTGCCGAACAGGTCTGGGCTGGGGAGCCCGA
GGCCGAGGGTCCAGAGACGCCTGCGGCTGGCGGCGAGAGGCGGCCCGGGCTTGCGGGCGGAAGTGCAAGGGCGGCCGCGTGGAGGCCCCGCGGG
CCGCGCGTCTAGGTGACCTTCCCCGGCGGCGCCCTCTGGCCCTGGATCCCACCAGCGTGCATCCGGCGGGGACCCGTTCGCCGCCCACGCTCGC
CGGACCAGGCCAGCGGGCGGGTGCCGGCCACGCTGCCGCCTCGGGGATGAGCTCACGTGACGACCGTGGCCCCGAGTCCCCAGGCCCACGCGCC
CTGGCCGGCCCACGCCCGCAGAAGCTGGCGCCCGGCACCGCCCGCCTGTGCCAGAGCCTTCTAGAGCGGAACGCGCCGGCCGAGAGTCGACTTA
GGCGGTAGAGGCCGGAGCTCGGGCCTGGGCTGAAGGTTTGGCCTCGGAAAAGGAGGCCCCACGGGGAAGGATGTGGCCTTCTTGGTCTTAGGAG
TGATTACTGGTTCGGTCGTTCATTCATTCGCCAAATGCGGCGGTGCATTCTGGAAACTGTTTCGGAGCCTGACTTTGGATTAGAGGCCCGCAGT
CTTAGTGGTTTGTACAAGGTCACACAGCTGGGAAGTCCCCATACCTTCGTTTTCTCATCTATAGGACGGGAGCAATTGTACCTCTCCTTCGATG
AGTTTTGGGGAATAATAAAATGAATTAATTAGCATGAAACCTTTAGAACAGTGCCGGTGTGGAGAAGGGCTAAGTAGAAACCCTGACACCCTTG
GGGCTTTTTGGGTGAATCCAGCCCATCCCCCGCAGACCATTTTGCTACACGTGGCGTTGTGGAAGGGGTTCAATAAATGTAGCTCCTGTTTTTA
AACATTCTTTCACTATTATTGACAAGCATTTGTATACCAGGTATTGAGTAACTTTCAGCTTAATTTATGAAAAATCTTGTAAGTTTACAAATGG
ACATTGTCAAGCTGGAGCAGTAAGTTTGCATGTGGAGATCTTGAATGCAGGAGCAGACCCTGGTTTAGAGCCCGTTCCGCCCGGGGTCCCTGTC
CCCTTGACTAGCCTCCCCTCTCCTGGGGAGATAGGGGAAAGGGCTCCTCTGTTGCCTAAACTTCTCCACTGAGGCAGGAGAAGGCCAGAGCAGA
CTACCGAGGGCCAGCCTTCATTCTGCCCACCCCAGCTCTGGACCAACTCAGCTCCTAAGGGTCAGACTGAGGCACTGAGGCCCTGTGCTACTTT
GTCTTCAGTGCCTCGGGTGCTGCTGCCTGGCACACAGTAGGTGCTTGAGAAGGGTCTGTGGGGGTAAATGCACTCTTGTGTCAGAATCCACCCA
TCTTTACTGACAGAGTTTTACTATTCCAGTACATGGCCCATAGTAGGTGCTTATTGAGTATTTGTGGAATTATTGAATGGGCATATGAAGGGCT
GCAGGCCCAGACCATAGTCCTTTGATCCTGGACTTTTTTTTTTTTTGAGACAGGGTCTTGCTCTGTTGCCAGGTCTGGAGCGCGGTGGCACCAT
AACGGCTCATGGCAGCTTCTGTTTCCTGGGTTCAGGCAATCCTCCCACCTCAGCCTCACAGAGTATCTGGGATCCCAGGCACACACCACCACGC
CTGGCTAATTTTTGTATTTTTAGTAGAGTCGGGGTTTCACCATGTTTGCCAGGCTGATCTCAAACTCCTGGCCTCAAGCAATCCACCCGCCTCC
GCCTCCCAAAGTGCTGGGATTACACGCGTGAGCCACCATGCCCGGTCCTGGACATCCTTTTGAGGGACACAGACATCTCACTTGTGGAAAACAG
CCTCACCTAGCAGAGAGTCACAAATATTTAAAGAACTGTAACAGTATGTATAGTCTCTTGAAATAAGTGCTTTAAACACATCGTCTTCATTCTC
TGGTTTTCCAAGTCCCGAGGGAAACTTGTTGAACTGGTGTTTCTGGGCTTGCAGGGAGGGTGACATGGGTGACTGGGATTCTACAGAAGGTGTT
GGGCCTGTTACCCCTTCCCTCATGGTGGGTGGATGGACAGCTTGTGCTGGGGTTGTCCTGAGTAGGGTGCCTGCCTGTGGGTAGTGGGGACTAC
TCCTTGTTGATTATTTAGTTTTATTTTTGCTATTCTAGAGTGGGGCTATAGGCAGGGGTTATTCGTAGCTCAGAAAGTGGGGGACCATACAGGG
ATGAGGTGCCTGTGGGCCCCTGGCCTTGCTGAACACTAGGCAGGTGTGGACAGTGAACTCCATGCTGGGGAGGTGAAGGGCATCCAACCTCTAC
CCTTACCAGGAAGATGCTCCTGAAGCACTGAAGTCCACTTCGCCTTTGATGGAATTTCCCGTGGTGTAGTGTCTTCGGTGGTAGTGACTGGAAG
AGGCGCATGTGTGGTTGAGGACCAGCCGAAGCTTGGAGGCAGCCCCTCCTGGTGCTCAGAGATTCTGTAGTCTGCGGCTTTGGGTTTTCCATGG
AGTCCTGTGGTAGCCACTGAGAAAAGTTGACCAGTGGGCTCCTCAGCTGGGTCTGGGCGCATTTCTCCATTTTACCACGTACTCAGGGCGTTTG
GGATGAGTGGGACTTTAAGTTCTTTACTCAGAACTGTGCTGGAAAGAGCACTGGAGAGTAGCTTAGGCCCATAACACAGGTCTGGTGAGCCATA
GGGTCGGTCTCATACAGGTCAAGTCACTGTCTTGTGCCTCAGTTTCTCTCTCTCTTTTTTTTTTTTTTTTTTTTAGATGGAGTCTCACTCTGTT
GCCCAGGCTGGAGTGCAGTGGTGCAATCTCGGCTCCCTGCAATCTCTACCTCTCGGGTTCAAGCAAGTCTCTGCCTCACCCTCTCGAGTAGCCG
GGATTACAGGCGCCTGCCACCATGCCTGGCTAATTTTTTTGTATTTTTTAGTAGAGATGGGGTTTCACCATCTTGGCCAGGTTGGTCTTGAACT
CCTGACCTCCTGATCCACCCGCCTCGGCCTCCTAAAGTGCGGGGATTGCAGGTGTGAGCCACTGAGCCCGGCCGAGTTTGTCTGCTATAAAAGT
ATGATTGTCGTCATTACAGTGATTGCTGATTGAGGGCTTGCTCAGCACCTTTCTGGGGGCTCAACGAATGTTCTGTGATGTTGAGTTCACCACC
CTATACCCTGGGAGAGAGATAGTGTGTTTCCATTTCACAGGTCAGCAGACTCGAGCACAGAGAGGTGAGGTAACACAGCCTGGCAGGAGTGGAG
TTGGGATTCAAGGCCTGGTCTGAATGGTGGTGCTCTCACATTGCAGTTGCACTCCAAGGGACCCTTGCAAGGTGCTAACAGATGTGAATGCCTT
TTGGAAAGTCAAAATTGGTGTTCAGATGGGAGACATTATTTTTCCACACCAGTCAGAGTGAACTTGTGCATGGCTGTGTGCGGGCCAACTGGCA
GTGGACAATGGAGTCACCTCTATTTCTTGCAAGGACAGTGGTGCTGAGTGTCTATTATAAGGGTCCGTTTTCCCTGGGATCTGGTGGGAGGCAT
TCTTTCAGGACCTGTTCAGCTAAACAATGCCTCATTGTGGACATGAAGGACTCTGGACCCTGTGGTTCAGGGTGACCTTTTGAACATGCTTAGC
AGCCATTATTATAATAAATATACAAAATGAAAACTTGTGATAAAATACTGTGTGTATTCCCTGCTTACCAAGCGCCAGCCACTGTACCAAGTGT
TGTATATTAACGAGCTAATTCAGTCCTCACAGTGACGCCAATCCAGGCTCTCTTTCTCCATATTAGAGCTGAAGAAAAAGGAGTAGAGAGGTTG
CGACTTGCCCAAAGTCACATACCTAGAAAGTGGTGGGGTGGGTTAGAACTCAGGTCTATTTGATTCCCGAGGCTTCCTGCCCAAGCACTGTCTG
TCTCAACCCTACCTGGTCAAGCCAAGCAGTGAGGCTCCAGGGAACTTGAGGTGCCTTAGGTCAGGTAGGAGCCCCAGGTATGCCTGGGATCCAG
ACCATAGTGTACTGGCTGCCAGCGCAGGCCATCTCCCCTTGATAGGTGGGTGGGCAGGCTGCCCTGAGTCAGCAACCTCCTGAGCCCAGGAGCT
CAGCATGGAACTCAGCTGTGCTGGTGGTGGCCCAGCTGCCAGGGTGTTTCAAAATAGAAGCTACCCCTGGAAAGCTCTACTGCCCACTGCGAGA
CTGTTGTGGAAGAATGGAGGGAGAGAACAGCTGCATTGGGTACGCAGACCTGCTACCAGAACACAGGGCCATCAGCCTTTGCCAGCATCCTGGA
GCTTGAACCAGGAACTGTTCCCCTGGGTTGTTTCTTCCTGGCAAGAAAGAGAAAGTGCTGGAGTGTGTGTGTGTGCATGCGTGCATGTGTGTGA
GCGAGCTGCTGATTGTGTTCGCCCCCATGTTGGCTTTTAGAGCTAGGGTTGCCTTCTGCTGGGGAAGTTGCTTTTCCTGGTCCTCAGGGCTAGG
AAGAGGTGCTGTTTCTGTGATCACTGGCTGGTCTGATACCCCTCCTATAGGCTGGCCTAGTTCTGCCCTGACAAACCACCTAGAATGGTGTTGG
TGAAACAGCGGGGCCAGAGACTTTTCCATAGTACTCTCTTCCTTGGCAGAATCATAAGATGATGGCAATGATGATGATGATGGTTGTGATTGCT
CTGTTGGTTAGCGTCTTTGCAGGGATCTGGTGCACACTTACACTTGGTAATCCAGGAGAGTTTAATGAAGACATCATTTGCAAAGCTGTGGATG
GGGTGTCAGGTCTGGTCTGTTAACACCCTGAGGCCTACAAGAGAGGGAGCTTCTTGGCAGGAGCTGTGGCCTTGGGTAGAGGAAGGTAGTCATT
GCCCATCTGAAGCCTAGCAGGGAGGGAGACTCGGGGTGAGTACCCCGACTTCATTCTTCCCTAAACTCCCGTCTTCTGCAAGTACCTCACGTTG
AAAGGCTAAGCCCAATCATAAGCCCATGGACAGCAGTCCATAATGATCAGTTCTGGGGGCACAGAGCAGGGTGGAAGGGAGGAGAATGGATCTG
GAGGGGCAGACAGAGTATCCAGTGTAGTGGATTATGGTTATTACTGCCATTTGTTGAACACTTACTCCATGCCAGATACTTCTGCTGTTCTGCC
TGTTTCACTCATTCCTTGTAACAACTTCCTTAGGCCTAAGGCCTTTCCTCCCAGCTGTTTTTAGCTGAGTCTTGGAGACCCATGGCCACCCTGC
TCCCTGTATCAACCATTGCAGTAGTTGTTGGCTCTCTAGGGGTGCAAAGCTCATCTGCCTGAATCCTTCTATTATTACTATTTATTTCTTTTTA
TTTTTTATTTAAAAGAGACAGGGTCTCACTGTATTGCCCAGGCTGGTCTCGAACTCCTGCATTCAGGCGATCCTCCTGCCTCAGCCTCCCAAAG
TGCTGGGATTAGAGGCGTGAGCCACCATGCCTGGCACATGCCTGAATCCTTTTGAGGGAGCCTCTGCAGAGAAGAGAGCATATTTCTACCAGGC
GGGGGCCTGGTAGGTGCTTAGTGCAGGAACCGCCCTGAGCTGCTGCTTCTCAGTGGCACCTGGGGGCAGCCCTGCCTGTTAGGCACAGGCTGAG
AGGTAGGGCAGGAGGAGTTCCATCTCTTCTGGCAGGCTCTTCTTGAACAAGCGCCTCCGGTGATTCTTGTGCACCCTGAAGTTAGGGAGCTCTT
TCCTGCTCATGATTTCTGGCAGGGTGGCCAAGCCACTAGTGGTCACCTGGGAGTGAAATGTGCCCCGACGGACCTTGCCTGATAACTCTTCCAT
CTCAGTGTAGTTTAAATCTGTTCTTGCAATCTGTATGGACACTCTAGGTAGCCTTCTGGCATTGCTACTGCAGCAGCCTTGGAACGTGATTTGG
GACACAACACAGCTCAATTTCAGGCACAGTGGTTGGCTTTTAAAGTGCTGTTTTTTTGGAGAGGGAGTCTCTCGCTCTGTCACCCAGGCTGGAG
TGCAGTGGCACGATCTTGGCTCACTGCAGCCTTTGCCTTTGGGTTCAAGTGATTCTCATGCCTCAGCCTCCCAAGTAGCTGGGATTACAGGCAC
TTACCACCACGCCCGGCTAATTTTTGTATTGAAATTCTTTATCCTTAAACAAATTATTAGTATACACAAACAGCTTAAATAATGAAGCAGAACA
AAAAATGAAAATTCGATTTGAAGTGCATTCCTTCCCTAAACCTCCTTCTCCAGAAGCGTATCTAAGCCAGCCCCAATTTGCAGATTGGCTTGTA
TCCTTCCACATGCTTTCCCATCCATTCATGAACATAGAAAGATGTGTAAAAAGGTTTTCCTCCAATTACATGGAATTACAAATTTTCTGACTTT
TTTCATTTAACTGTATCATAGCTAGCTAGCTTTCTTTCTTTTTTTTTTTTTTGTTTCGAGACGGAGTTTTACTCTTGTTGCCCAGGCTGGATTA
CAATGGCGCGATCTCAGCTCACCACAACCTCCACCTCCCAGGTTGAGGCAATTCTTCTGCCCCAGCCTCCCGAGTAGCTGGGATTACAGGCATG
CGCCACCACGCCTGGCTAATTTATTTTGTATTTTTAATAGAGACAGGGTTTCTCCATGTTGGTCAGGGTGGTCTCGAACTCCTGACCTCAGGTG
ACCCGCCCACCTCGGCCTCCCAAAGTGCTGGGATTACAGGCATGAGCCACTGCGCACGGCTATCGTAGCTTCATAGCTAGCTTTCTATGCCACC
ACATACATCCACCTTATTCTTTTTTTTTTTTTTTTTTTTTTGTGAGACAGTCTCGCTCTGTCATCCAGGCTGGAGTGCAGTGGCATGATCTTGG
CTCACTGCAACCTCTGCCTCCTGGTTTAAGCTTGTGCCTCAGCCTCTCAAGTAGCTGGCATTACAGGCGTGCACCATCACACCTGGGTGATTTT
TTTTTTTTATTTTTTTTTTATTTTTAGTAGAGACGGGGTTTTGCCATGTTGGCCAGGTTGATCTGGAAGTCCTGGCCTGAAGTGATCCGCCCAC
CTCAGCCTCCCAAAGTGCTGGGATTACAGGCATGAGCCACCCACCTTATTCTTTTTGATTACCATATGGTACTCCTTAGTTTGCAAGTATAGTA
CCTTAATTTTTATTCATTCATTTTCCTTTTGATGGACTTTTAGGTTGCTTCTGGGTTTTGTGTTACAAATACTGCTGCAGTGAGCATCCTCATA
TACACATCTTTGAGTAGTGCCTTGGTTGCTACAGTGGGGCAGGTTCCTAGAAGTGGAATTGCCACTAGGGTTAATGCTGGGTTAATTGCTCTCC
AAAAAGAGTGTACCTGTAGTACTCCCCCTTGGCGTGAACAAGAGTGACTGTTTTCCACACCCTCACTGACACTGGATATTATTTACGTTTTTGC
CAGACTGCTGGTTAAGTGAGCTTCTGAAACACAGATGTGGTAGTCACTCAACAGTGGCTCTTGGTGGCCCTTAGAATAAAATCCATGCATGGTA
TTGAGGTCAGAAGCAACCAGTGCTTCTGATGGCACTTGGTCTCCTCAGCCTGAGATCAGCTCTCCTTAGGCCGAGGTGGGAGCAGGCAGGGAGT
GGAGGTCATTGCTAGTTGAGGGTCTCCCCAACTCCCCCACTCTTGGTGATGCTGCAACAGTGAGCCTGCAGAGGGAGCTCTTACTTCCTGGTTG
CATTGTAATGAGTTCCTATTGTCAGATTCCAAGTTTCTACTTTTTTCCCCATTGCTAAATAATACTTTGATGCTCATTGCTCATTCACTCATCA
AACACATTTATATTGAATGCCTATTATGTGCCAGGGCTTAGCATCTTTATATACGTAACTTTTTCCATATTTTGGATAAATTTATGCATGACAG
GCTCTGGGGTGCTTTGAATTCAAAGAGGTGATTTTGGGGGTAGGTGTATGGGGTAGGGTGGGGTGGGGGCACGGTGAGCACAGCAGCTGTCATT
GCCATTCAGCTGATGAGCCAAGAAGGATGTGTACCCCCAAGACAGAGGTTACGTAAGTATTATGCAGACTGTGCTGGGTGATAGATACACTATC
CTTCCCCTCTGTGCTTCCAGTATGGGCCGGATTTTCATTCCTCAGCTGGAATGGGGCTTGCATAATTTGTTCAAGGTCAGCCAAGGAAAGGTTG
GGTAGGTAGACTGGCAGGCTGTGGAGGGGAACCAGGGACTCCTGTATCCTGGTGTGACTTGCTGGTCCTTCTCAAAGTCTTCAGTGACTGCAGG
CAGGTCTGCTCAGCCTGGCCCTGCCCATCCCTCTGTTTCCTACCAACCAGTGGGAACAAAGCCCCCAGGCTCTGCCCTGAAGCCATCACTCAGG
CAGAGAGGCCTGTGGTTCTGACTCCCCCAGGACGCCCTCTGACTTCTGTCTGTCCAGGGCTAGCTGCTTTGCCGATGCCAGACTGCCCTGCTTC
ATGCGCAGCCTCTAGAGGCTCAGCTTGTGAAGGCCCCTGGGGGGTGGATGCTGCAGAGACAGCTGTGTATCAGTAGCTAACTGTACAGGCTCCG
ATTTGGAGCAGATCTGGGTGGAAGGAGTGACTGGGAGGGGTGGGGGCCAGGGTTCAGCCTTCAAGTCAGCACCCTGGCAGGGTGTTAAGTGAAA
GCAGGCTTTGTTGCCGACTTTTCTTTCCTCCAGTCCTCGCCTTAGAAAATTAATGCAGATTATTATTTCTATCATAAAATTGTCTCTGAAATGG
GTTCGAGAGCTGTGTAAAAATGGGCATTTTGGGGGTGCTGTCTGTAGCTGTAAGAAAGCTGAACTTCTCGGAGCGGCAGAGCTGTCTCGGGCCT
TCCTTAGTTCCGTCAGATCAGTGCAGCAGGGGATGGGCCGAAGTGCACAAAAAGGAGGGGAAGCTACCACTACCTGGCAGTTGGGGGCCTTTAG
TGAAAGGGTGAGTCATCCTTTTGCTGTCCTCGGTGGTGTCACTTCAGGCCCAGGAGTGGACTTGACTCCATTCAGCCAGTGGTTTCTGTGAGTC
TTCCTTGCCAGGTGCTATGCTGGGGAGAATGCTTACTTGGGGGCCCAGAACGGGGGAGATTATGCTTCATTGTCGGTGTTCAAGGAGATGGGGA
AGAGAGCAAGACACAGATGAGAAAGCCAGCATCCAGTATGTTTACAGTGAGGCATTGGATAGGGATATGTTTTAGTGAACCAGCAAGTTCACTT
GCGTGCTGCTGGTACCAACTACATGGTTGTGTTGTGGAACCCCACAGCCCCAAAAGAGCTTGGCCGTGTCGGAGCTCCTAGACGCCCCAGGAGG
GGTCCGGTCTGCTCTTCTGAGCTCTGCTCTCTGAGTGCCTTGTCCTTGGGTGGTAATCTCTTTAGTGGCCTTGTACTTCAGGCTGATGTTTGCT
GTGGGTGGGGTGTTTTTTGACAGGTGGCTGTGCTTGTTGGATTTTGGGGATTAGCGATGAGAGAGGGCATTTGGGGAACATTTAATGGATGAGT
GCTCTGCCTTTATCCTGAGCGGGGACCAGTCCAGATGTGAAGGCTGCTGTGTCTGGGAGGCGTCTCTTCTGTGGGGTCAGGGGAGAGTCGTGGG
GAAGGCTCTGGCTCCTAAGGTCCAGGGAGGTCTTAGGTGACCCACAGATATTACCACTGGACTGCTTCTCCTTCTGCTTCCCCAGCCCCCACCC
CGGCTGAGTTTGTGGCCCCCCTGGGAGGCATAGCACTGAAGAGGGGTACTTGGTTCACTGATGGTTTGTTAACCCTGTCGGGTGGAGAATGTTC
TGGGCCCGTTACTGTTATGTTCTGTGTCTGCCTGGGGTTACATTGCCTCAGCTTTCAATCCAGAGTTGTTCTCCTAGGAAGGTGTCAGGTAAAG
GGTATTTCTTGAGGTTCTGGTGGGTGGTTTCCTGGTCTATCCCGGGGGGATGCACCTACAGGGTTAGTTTGGGAGGGAGGAACAAGTGGGGAGA
GGGGAGCCCCCAGACTGGAAAGAGCCAGTGGCGGGCAGTGTAAAATGGATCTTCCCCCCTCATCTCTCTACCCTGATACTAATGTGGGCTGACC
TGTCCATGGTTTCAGCCTGATCATGTGCAAGAAATAACACAGCAGCGACAAAAATAGGACAGAGTAAACAGCTCGCTGCTCTTCTGTCTGTGCT
CTGACACAAAAGTGAGCTGGGATAAAATTAACAAAGCATCTGGAAAAAGTAAAGTAGGACCTGGATCACAACTTCACAATTTAAAATTGTAGAA
GTTTATACTGTTTCTTTTACAACATACTCAATGAGTCTGTGGCCTCCTGCAATGCCTTTGAAGTTAAAGCTTTGGCCAAGAATGCAACTTCCAT
TGATGATATGGAAGCATATCATGTCATATGACATGCCAGGAGCATGTCATATATGTGTTTATTTGTGTGAATATATATACATGTAACTTCAGAT
GGTATATATGCAGATGAATTTTGATAATTTAGGTATACTGTATTTTAAAACGAATGGAATGGTCCTTCTGGTTGAGACTAATGATTCTGGGGAT
GGAGAAGGTGCAGGAGATGAGGCTTTCTCATGCATGACCCCCTCTCCTCCTGGGCAGAGCACACCATGTACAGTCCCAGATTAGGATATTAATC
CTGTGTACACAGCATATACAGCATGCCAAAGCAAAGCCTTCCAGGAATGCAGTGTGGTCCTTAGGGCAAAGACTATTTGAGTTACTCTCCTTCC
TTGAACACCGAGGTGCTTAATAAATGTTTGTTGATTGACTTAGTGATATGCAGCAATCCCAAGTAGTCCTTTGATGGTTGGTTTTAAAAATAAA
GTTACACTGGAGCTCAATAAATATTTGTTTAATGTATTTGGAGGATGTATTTGGGTTTTTTCCATGTGAAGAGCTGCCAAACTGGTTTGGTTTT
ACTGTATTCTCTATGAGAAATATGAAGTGGGTTCCCATAAAGTCCAAAAGTTTTGTGAAAAGTTGGGCCCATCCTGGCATATAGTAGGTACTCC
ATAAATATTTGTTGAATGAATGAAATCAGAGCCACATGGAAAATGATGTTCCCATGAAAAGCTCTGATTTCTATTCGGTACATTTAATGTCCTA
GTAGCTGACATTTTGTAAGATGATATGTTTTATTGGCAACAGTCCTACAATTCAGTAAAGTCACTGGCTCATGGGAATGTGTCTTTGACAGTTT
ATTCATTAAGCAAATATTTCCTGACTCCTACTCTGTTCTGGGCCTGGGCTCAGTCCTGGGGATGCTGAGGAGGAGAGGTCTAAATTGTTGATCA
TCTGTCAACAAAGACTCATTGAGTGTCTCAAGCTCTGGCCACAAAATGAAGGACAAGACAGCAAGGGCTTCTTGGCCCATCTGGGAGGATAAGC
TACCTGGTAGTCTTAGTGCTAGGTGATTTTTATTGGTTGCTAATTACATCTAATAATTGTTATTGATGGAAAGCTAATTTCCCTAATTATCAAT
GCATTTATTCTGGTGATTCTTTTATCATAGCAGAAGATGGCTGTGCCACCCACGTATGCCGATCTTGGCAAATCTGCCAGGGATGTCTTCACCA
AGGGCTATGGTGAGTGTTGCAGAGAGGGGGTGCCTTTCAGCTCACAGACTGAGTTGGCATGTTTGAGACTTTACCGATAACTGTTTAGGAGACC
TGGGTAGGTCAGCTGTTCTGAACTTTCACGTGAGGGAAAAAAGAAGCTTTCATTTGTGTGGCAATTGTAGCAACTTTGGTTGTGCACCCAGTTG
GACAGGCTCCTGCATTGGTGCTGTCAAGTGGGTATTCTTGTTTTCTTTTCCAAGTGTGGAGCAGCCCTGTGGAAGCTGCTGCAGTGGAGCGCGG
CTTCTATATTTAGCCTGTGCTTAGCCACATGCCCACGAGGCCCAGTAACTGTGTCTTTGCCAGCTCTCAGGTGTCTTCTTATCAGACCCTGCTG
GGAGTCCAGGTGGGACCCAGGTGGGGGTGCCCATTCTGTCTGCTGCTGTTCCATGGCAGCTGAGAGCTCCCCCCACGAGCAGCAGCAGAGACTG
GGTGCCCTCAGCCCCTGTGGCAGGAGTGTGCTTCTTTTTAAAAAAGCATCCTGAAGCAACCATGAGGGAGGCAGAGGTGCTGTTCTCTAGTTAT
TTAACCTATTGACAGTTGTCTTCTTCTTTCTTTAGGATTTGGCTTAATAAAGCTTGATTTGAAAACAAAATCTGAGAATGGATTGGTAAGTTGT
CCAACCCACATGGAACATGAGTATTTATTTATTTTTATTTTATTTTATTTATTTATTTTTGAGATGGAGTTTTGCTCTTGTCGCCCAGGCTGGA
GTACAATGGCGTGATCTCAGCTCACCACAACCTCCACTTCTTGGGTTCAAGCGATTCTCCTACCTCAGCCTACCGAGTAGCTGGGATTACAGCC
ACCCGCCACCATGCTCGGCTAATTTTGTATTTTTAATAGAGACGGGGTTTTTCCATGTTGGTCAGGCTGGTCTTGAACTCCCGACTTCAGGTGA
TCCGCCCACCTTGGCCTCCCAAAGTGCTGGAATTATAGGCATGAGCCACTGCGCCTGGCTTATTTTTATTTTTAAAAAAGATTTTAGATTCAGG
GTGTACATGTGCAGGTTTGTTACATGGATGTATTGTGTAATGGTGAGGTTTGGGCTTCTAGTGTACCCATTACCCAAATAGTGACCATTGTATG
GAACGTGGGAATTTGTTGTAGATCAACAAATAACTGTCGTGACCTGGCCCGCAAAGCCAAAAGAAGGTACTTTTTTTTTTTTATTCTGGCAGTT
TTTGTGGTTTTCACATTCATTCCATTTTCTGCATGATGGGTGGTCCAGGGGAACCATGCCAGTAGGCCCCCAGTTCAGCAGCGGGTAATAGAGG
AGAATGGGTCAGGTCTCAGTTTTAGTTTTCCTTGTGCTTCTTATTTTCAAATTCACCTGCATTCAACCACATAAGTGTTTTTAAAGTAATCTTA
GTAGTCCCTTTGAAATTATGTGTGCAGTAATTGTTGGAAGAAAGAAGTTTTGGGTTTGAATTACCGCTGTGTCAGCTTTAGCTCAGTGCTTTTT
CTCTGTACTGCCCTGGAGCCAGGATGGAAGGGAGATGCTTTCCTGTCCAGACTGAAGAGTTACAGGGAGGAAGGAAGCTGTCTGCTGACAACAA
ATTCCCCATAAGATTTCTTTACATAGCAGGCTCCTTTACTCCCATCTGTACTTTAAATATCTATTTGAATTGTAGAGTTAGCCTTGGCCCCCCC
ACATTCAGGGTCTTGCCTCTTGCAGAAAGGCAAGTATAGTGTTTAGGAGTGAAGTGAAGCCAAGTGAGTGCTATGGTGCAGGCTGTGACTCTTC
TCTCTCCTTTGAAGGAATTTACAAGCTCAGGCTCAGCCAACACTGAGACCACCAAAGTGACGGGCAGTCTGGAAACCAAGTACAGATGGACTGA
GTACGGCCTGACGTTTACAGAGAAATGGAATACCGACAATACACTACCCACCGAGATTACTGTGGAAGATCAGGTAATGGCTGCTGTGAAAAGA
TGTGGATTTATTAATTCGCATCTGCCAGCCTGCCTTGTGACTAAATCTAGTGGCAAATTTCTTTTCTGTTCAGCTTGCACGTGGACTGAAGCTG
ACCTTCGATTCATCCTTCTCACCTAACACTGGGTAAGAGTTTCATCTGTTTTCTCCACAAGGATGTTCTCTGATGTTGCAGGTGGTGGAAGCCC
TTTGTGACAAAATGTTTTGGGGGCTGACAGCACCTAAAATATGGTCTCCGAGACCTGGCGATTTAAGTCCTCTGGTTTTGATTTGGTCCCAGGG
GTCGTATCCACATCTATGCACTAACAGACTATGCTATAATGAGCACTTCCTAATTCAGGACTGGTGTTTACCCAACAAACGATTTCTAAGTGCC
ACCTGTGCTTGGCCCTGTGCCCATCTGGAGATCTAAGGGTGCCCAGGCCATGATTGTTACCCGCCAAGGAGCTCACTTTCTAATAGAGGATTCA
GACCAGTGTTTAGCCGTGTTTTGTAATAAGATTCTGCATTTTGTTAGTGTTCTCCTAGTTTCTGCCATGGAGGTTGCAGGATTTCTGTTCAGTA
ATAGCCATTTCATGTGAGTTAGGCTGCTGGTGATGTAAGTGGCTATGACCAAACGGGAGTGGGTGACATTTGGAAGCAGGGCCTAACTGGTCAC
TGTTGCCAGCCTCATTTCTTCAGGCATGGAAGTCCTTCTCCCAGCTTCCTGCTGCTTAGGGACACTGAGACCCTGTGGGTGTAACTGCAAGGTG
GCTCCTGAGGCTGCACTTTCCCCAGGCCTTCTGGCCACAGATGTAATAGGCTTGTTAGTTCTTGACCATCACTTGGCATGATAGCATGGTAGTC
TGCCTCAATTCCTGGGTGCTTTTCAAAGTGCCTAGAAGGAATTATTCCCTTTTAGAATACACCCTTGTTTTTTTCCACTCATATTGGTGGGTCT
TCCAAACCTGGGGAAGATGTTTTATATAGTGATGTTAGGGTCAAGGGCATGTACTGAATTTCAGTTTTTAAAAATTGTGGGCCGGGCGTGGTGG
CTCACGCCTGTAATCCCAACACTTTGGGAGGCTGAGGCAGGCAAATCACCTGAGGTCGGAAGTTTGAGACCAGCCTGACCAACGTGGAGAAACC
CTGTCTCTACTAAACATAAAAATTAGCTGCGCCTGGTGGGCGCATGCCTGTAATCCCAGCTACTTGGGAGGCTGAGGCAGGAGAATCGCTTGAA
CCTGGGAGGCAGAGGTTGTGGTGAGCCAAGGTGGTGCCATTGCACTGCACCCTGGGCAAGAAGAGCGAAACTCCATCTCAAAAGAAAAAAAAAA
AAAAGAGTCGGATGCAGTGACTCATGCCTGTGATCCCAGCACTTTGGGAGGCTGAGGTGGGTGGATTGCTTGAGGTCAGGAGTTTGAGACCAGC
CTGGGCAACATGGCAAAACCCCATCTCTAATAAAAATACAAAAATTAGCCAGGCATGATGGTGCGTGCCTGTACTCCCAGATACTTGGGATGCT
GAGGCATGAGAATCGCTTGAACCCAGGAGACAGAGGTTGCAGTGAGCTGAGATCGCACCACTGCACTCCAGCCTGGGCTACAGAGAGAGACTCT
GTCTCCAAAAAAAAAAAAAAATTGTGGTAAGATATACATATAAAACTTAACATCTTTAAGTGTGTAGTTAAATGGCACTATGAATTTTTCAGTT
TTGCCATTCATTTTTTTTCTTTCTTCCTTTTTTCCTTTCCAACAGATATTTATTAGAGGGAGGATCTACCAAGTGCAGACCTGTGCTGGGTGTG
CAGTGCACACAAGTGACTGGGCACACAGGGTTCCTATCCTCTGGAGATGTCGTCTTATAGCTTGGCTATAGATGATCTGAAGAGACCATTGTTA
CGGGGCTGATTTACTCTCATTCCTCTTCGCTGCTGTAGTTTCTCCTGCAAGAGCACTGCCTTTTTGCAGTGCTGTGCACTCTCCCCCCAGGAAC
TCTGCCATGGTATCAGGGAGCGGTTATCTGGAAAGATCCTGGGATAGGATGAGAGGCTGGAGGATGTGCCCACATGCTTGAATTTGCTGCCTGG
GACTTTGCCTGCTGCCCTGGGATAGCACACTTTGGAATTTGTAGGCGATTTCCTTCCCATGACCTTAGGGAGCTTTGCAGAGGTCTCCAGGCCA
TGCAGTATAAAACACAAAGGAAAGTTTTTTGGTTTTTTTTTTTTTTGGGTTGAGGGGGACGGAGTCTTGCTCTGTCGCCCAGGCTGGAGTGCAG
TGGTGTGATCTCAGCTCACTGCAACCTCCTCCTCCCGGGTTCAAGTGATCCTTTTGCCTCAGCCTCCCAAGTAGCTGGGACTACAGACACGGGC
CACCATGCCTGGCTAATTTTTGTATTTTTAGTGGAGACGGGGGTTTCACCGTATTGGCCAGGCTGGTCTTGAACTTCTGACCTCGTGATCCACC
TGCCTCGGCCTCCCAAAGTGCTGGGCAGATTCATGTGTTTCTCCTGCCTCAGCCTCCCGAGTAGCCGCCATTACAGGAATGCACCACCACACCC
AGCTAATTTTGTATTTTTAATAGAGACAGGGTCTCTCCATGTTGGTCAGGCTGGTCTTGAACTCCTGACCTTGGGTGATCTGCCCACCTCAGCC
TCCCAAAGTGCCGGGTTTACAGGCGTGAGCCACTGCACCTGCCCCCCTTTTTTTTTTAATTTACTTTTTATTTCAAAATAATTTTATACTTAGA
AAAAAATGTGGCAAAAATAGTATGCAGAGTTCTCCTGTGTTCTTCACCCAGCTCCTAATGTTAATGGTTTACGTAACTATAATGATCAGAATCA
GCAAGTTAATGTTGATATCATATAGTTAATTAATCTGTTGTCACAATGACGTCTTTTTTCTGATCTGTGATCCAGTTCAGGTTCCTAAATTGCA
TCTAATTGTTAAGTCTTCTCTAATCTGGAATATTTTCTGACTCTGACACTTTTGAAAAAGAGTGGCCAATTATTTTGTTAAGATGTCCCTCAGT
TTGGATTTGTCTGATGCTTCCTTATGATTAAATTTAGGTTATGCATTTTTGGCAACTGTAGTTGTTGTGTTATATTCTTCTCTGCACCTGATGT
TAGGAAGAATATATCAGTAGGCCTCATTTTTGGTGATCAGTTTTGAAGTGGTGTCTGCCAACTCTCTTCATTGTAAAGCTACTATTTTTCTCTT
TGTAATTAGTAAGTATCTTGTGGGTAGACACTTTGAGATTATGCAGATATTCTATTTCTCATCATACTTTTGCTCAAAAATAGTAACATCCATT
GGTGATTCTTGCCTGCAACAGTTACGCTTGGCAAATGGTGACTGGAGTGCAGTGGTGTGATCCTGGCTCACTGCAACCTCTGCCTCCTGGGATC
CAGCAATCCTCCTTCCTCAGCCTCCCAAGTAGCTGGGACCACAGGCGTGTGCCACCAAGCCCAGCTAATTTTTGTATTTTTAGTAGAGATGAGG
TTTCACCAGGTTGGCCAGGCTGGTCTAGAACTTCTCAGCTCAAGCGATCCACCCGTCTCAGCCTCCCAAATTGTTGGGATTACAGGTGTGAGCC
ACCACGCCTGGCTACAAATGGTGATTTTCTTTTCTTTTTTGTTTTTATGAGATGGGGTCTCACTCTGTCACCTAGGCTGGAGTGCAGTGGTATG
ATCTCGGCTCATTGCAACCTCTGCCTCCCTGGCTCAAGGGATCCTCCCTCCTCGGCCTCCCCAGTAGCTGGGACTACAGGTGCCTGCTGCCACC
CCTGGTTGATTTTTATATTTTTGGTAGAGACAGTGTTTCACCATGTTGCTCAGTCTGGTCTCAAACTCCTGACTCAAGTGATCTGCCTCCCACC
TTGGCCTCCCAAAATTCTGGGATTACAGGTGTGAGCCACCACACCTGGCCTCAAATGGTAATTTTCTATTGCCATCCTTCCTTCACTTGTTAAT
TGGTATTCTACCGTAAGGAAGAGCTATTCCTTCTTCCCCATGTATTTATTTAATCAATTATTTATTTATATTAGTATAGATGCATGGACATTTA
CTCTATGGGCTATAATGCACAGTCTCATTTATTTTGTCGCTCAAGTTGTCCCAGATATGGGCTTTGGGAGAATACACTCTGTGTGCTTTTTGAC
AAACCTCTGTCGTTTTTCAGAGCATGTCTTTATTTCTGGTTTTAGAAAGTGTACCACGCTCATCTAGTACTTGTTTTTCCCCAGTCCTGCTATC
TGCCATTTCTCCAAGGAGCCTTGGTTCTTTATGTTGGAGAATGATATTTAGAAACTAAGATTGTAGGCCTGGCGCGGGGGCTCATGCCTGCAAT
CCCAGCACTTTGGAATGCTGAGGCAGGTGGATCACGAGGTCAGGTGTTTGAGACCAGCCTGGCAAAACCCTGTCTTTACTAAAAATACAAAAAT
TAGCTGGGTGTGGTGGCGCACACCTGTAATCCCAGCTACTCAGGAGGCTGAGGCAGGAGAATTGCTTGAACCCAGGAGGTGGAGGTTGCAGCGA
GCCGAGATGGTACCACTGCACTCCAATGTGGGCGACAGAGCAAGACTCCATCTCAAAAAAAAAACAAAACTAAAATTCTGGGTGCTAGATGTAC
ATAATGCTACTGGGTATTTGAGGAAAGTTCTGGTGGTGCATCATGGAGATTAGAAACGAAAGCTGGCTTTCCAAAGTGACTGAGGGTACAGCCT
GGGAAAACACCTGGCTCCCAGGAGGAGCGGAGTGTAGACCACATCTGGATGAGGCCACAGACCTCAATAAGGACCAGCACCCACGCTGAGTATC
CATCACAAATAAAGCTTGCTTGTTAACCACAAATCAGTCAGGCCTCTCTCGGGTGCTTGTCAAATTAGCACTACTGTTGAAATAGCTTAAAAAT
AAATCCCAATAATATCTGAAAGAGGAAGACTCCACAGACATGGTGTGCAGTGTTAGAAAGAGACCTGGAGAGAAGCCTGAGCCACTCCCCAGCA
CTGCCCTTTCTCTTGGGGATTGTGGGCCCTGGCCAGAATCCAGTGAGACAGTGGGTGGGGGTGCCATGTCAACTGGTGCAGGTGGTGCACACCA
CTGGTCAGAGGCCCTGCCATAGGCATCATAGTTTGTGATCATCAGCAGCTTTTTATTAAAAATTGAAAAAGGAGATGCTTAGAAGATGCCTGAT
TTTATATTTCCTTTTCCCTTTGTAAATGTCAGCCTCTCAACAGCTGTCTAATCATCAGTCATCTCAACCAGCCTGGTGCCAATCACATACTTGG
CTGAAACCCAGACTTCTTTCCCCAAAGAGCAGGCAGATGAAGATAGGTTTCTTTTTCTTTTTTCTTGAGACATCTTGCTCTGTCGCTCAGGCTG
GAGTGCAGTGATGTGATTTCAGCTCACTGCAACCTCCACCTCCCAGGTTCAAGCAATTCTCGTGCCTCAGCCTCCTGAATAGCTGGGATTACAG
GCACATGCCACCACGTCCTGCTAATTTTTGTATTTTTAGTAGAGACAGGGTTTTTCCGTGTTGGCCAGGCTGGTCTCCAACTCCTGACCTCAAG
TGATCTGCCCTCCTTGGAGTGCTGGGATTACAGGTGTGAGCCACTACGCCTGACCAGTTTTGGATTTTTCTTATTGAGAGATAATTTGGTTGTT
ACTCCTTTCCTTCAAACCTTGCCAAAATGTACAGAAGTAACTTGAACAAAATTGAGGTTCCAAGGCATAAGTTTCATTGTCACTTGTTCTGTTG
GTGAGCAGGTCTGTGCACACCTACTCCCAACTGCTGAGGGAGCTGAGCAGCCAAAGAAAGAGACTGACAAATCCAGTTTCTCAGAAAGAAACAT
TTAATAGAGACTTACAAATAGAAGCGATGTCTCAAGCAGCCAAGAGACAGTGGATCCCTGCATTCATCCTCCAGAAAGTATTCTTTATATAGCA
AGCTTTTAGAGTAAAGACATGTGCATGTCTTAGACCATCTTGCAGAAAACTCTTGCAAAACTTGTGACCAAAGGGAGGTTAGATAAGCATCTTT
ACTTTTTAAATTTTTATTTATTTATTTTTTTGAGACAGTCTCACTCTGTCACCCGGGCTGGACTGCAGAGGCGTGATCTTGGCTCATTGCAACC
TCTGCCTCCCGGGTTCCAGCGATTCTTCTGCCTCAGCCTCCTGAGTGTCTGGGACTACAGGTGTGCACCACCACACCCAGCTAATTTTTGTATT
TTTAGTAGAGATGGAATTTTGCCATGTTGGCCAGGCTGGTCTTGAACTCCTAAGCTCAAGTGATCTACCTGCCTTGGCCTCTAAAAGTGTTGGA
ATTACAGGTGTGAGGCACTGTGCCCGGCTGGCATTGTTTCTTTTATGGGATTGTCTATGATGCATTCCTTGGTCGTCATGGCTGTATTAGGCTA
TTCTTGCCAGGCAAGGTGGCTCACGCCTGTAATCCTAGCATTTTTGGAGGCTGAGGCAGGCAGATTGCTTGAGCCCAGGAGTTGGAGACCAGCC
TAGGTAACATGGTGAAACCCCATCTACACACACACACACACACACACACACACACACACACACACACACACACACGCTGGTCATTGTGGTGAGC
GCCTATAGTCTTGTCCTTGCATTGCTCTAAAGAAATACCTGGCCAGGCGAGGTGGCTCACACCTGTAATCCCAGCATTTTGGGAGGCTAAGGCA
GGCAGATTGCTTGAGCCCAGGAGTTGGAGACCAGCCTAGGCAACATGGTTAAACCCCGTCTACCCACATGCACACACACACTAGCTGGGCCTGG
TGGTGCGCACCTATAGTCAGTTCTACCTACCTGGGAGGCTGAGGTGGGAGGATCACCTGAGCTCAGGAGGTAGGGGCTGTAGTGAGCCATGATC
GCACCACTGCACTCCTGCCTGGACAACAGAGTGAGACCTTGTCTGAAAAAAAGAAAAAAATACCTGAGGATAGCTAATTTATGAAGAAAAGAGG
TTCAATTGGCTCATGGTTCTGCAGTCTGTACAAGCAGAGCACAGGCATCTGCTCGGCTTCTGGGAAGGCGTCAGGAAGCTTTTAGTCATGGTGA
TTGATGAAGCAGGAGCAGGCACATCACATGGCAAGAGCAGGAGCAAGAGAGAGAGAGTGGTGGGCAGGTGCCACACGCAAACAACCAGATCTCA
TAACTCATTATTGGGAGGACAGCACCAATCCATGAGGGATCCACCCCCATGGTCTAACCACCACCCAGCAGGTCCAACACTGGGGACTGCATTT
CAACATGAGATTCAGAAGATACAGCATCGAAACTGTATCAGTGGTGATTTTGTTTCAAGACGGTGTCACTCTTGCCATGGAACAGGCTATTTTC
CTACATCACTTGGGAGGGGTGCTGGCTTTATTTTCAGCTGTGACCTCTAAAATTCTTGTCTCATTGAGAATTTGGAATTTTTAACCTTTATTTT
TTTTTAATGAGAAATCTGAAACATACAAAAATATACAGAAAAATATGTTTAACCCTCATTCTATTCCTATCACACAGCTTTGATAGTTACCAAC
CTGTGGCCAATCATGTTTTATTTATACAGCCATAAATATAACACTCCTCCACGCACATACTATTTTAAGTAAATAACAACACTTAATTTCAGCT
GTAAATATTTTATTATGTATCTTTAAATGGCAATAATCCATTTTAAAGGAGCATAACCATGATATTATTATTATATTGAAAAAAGTTGATCATT
TCTTTTTCTTTTTTTTTTTTTTTAAAGATGGAGTTTCACTCTTGTTGCCCAGGCTGGAGTGCAATGGTGCGATCTCAGCTCACGCAGCCTCCGC
CTCCCGGGTTCAAGTGATTGTCCTGCCTCAGCCTCCTGAGTAGCTGGGATTACAGGCATGGGCCACCACACCCGGCTAATTTTGTGTTTTTAGT
AGAGACGGGATTTCTCCGTCTTGGTCAGGCTGGGCTCGAACTCCCGACCTCAGGTGATCCGCCTGCCCCAGCCTCCCAAAGTGCTGAGATTACA
GGCATGAGCCACTGCGCCCAGTGGATCATTTCTTAATATTGTCAAATCTCTAGTCTGTTCAAATTTACATGATTGTTTCATGATTTTTTTTTTT
TTTTTTGAGACTGAGTTTTATTCTTGCTACCCAGGCTGGAGTGCAGTGGCGCGGTCTTGGCTCACTGCAACCTCCACCTCCTGGGTTCAAGTGA
TTCTCCTGCCTCAGCCTCCCAAGTAGATGAGATTACAGGCATGTGCCACCACACCCAGCTAATTTTTGCATTTTTAGTAGAGATGGGGTTTTGC
CATGTTGGCCAGGCTGGCCTTGAACTCCTGACCTCAGGTGATCCACCCACCTCGGCCTCCCGAAGTGTTGGGATTATAGGCGTAAGCCACCGCA
CCTGGCCTTTTGCTGTTTTTAAAATAACAGGTTGGTTCTTTAGTCTCTAAAGATGACAGCTATGTGATTTGAGCGTTATTATAATTCTTTTTTT
TTTTTTTTTGAGACGGAGTCTTACTCTGTCGCCCAGGCTGGAGTGCAGTGGCGCGATCTCGGCTCACTGCAACCTCTGCCTCCCGGGTTCAAGC
GTTTCTCCTGCCTCAGCCTCCTGAGTAGCTGGGATTACACACGCCCACCACCGCGCCCAGCTAATTTTTGTGTTTTTAGTAGAGACGGTGTTTC
ACCATGTTGGCCAGGCTGGTCTCGAACTTCTGACCTTGTGATCCACCTGCCTCAGCCTCCCAAAGTGCTGGAATTACTGGCATGAGCCACTGCG
CCTGGCCCCGAGTGTCATTATAATGCCTAGAATAAACATACTGTTTAATATGTTTCAGCCTATTGCTAATATTTTTCTTATTGAGGCTCACATT
GCTCCATTGTCGAAGCCCTTGGGAGCCTCTTCAGGTTACCTTGTAAGTCCTTTTGCCTCCACCCCAGTAGTCTTCAATAGCTTTGATAGATGTT
TTAGGTTACTGTGTACATTTCCTGCCTAAGATCTGTAGTTGGCCATTTCTCCAAGGAGCACTGGTTCCATTTAGGAGGAAATGGTCTAGGTGCT
GAGGATGAAGCATTAATCTCAATGTGAATAAAGCTAGAAGTGAACAGTGCTGTGTAGTCCTTGAGGCTCTGAGTGGATCTGGTTCCTCCTGTGG
TCTCTGCTGAGAATGCAGCTTCCTGTGGGAGATGTCGATCTTGTAACTGAATGATAGAGACAATCAGATAAAAATGATCCATGTTGTATTTGTT
GGATGTGATCTTGTTCTCCAGTGTGTACTTTTGGTAAAAGACATGGTAATAATAGGTCTGCAGGTGACACTATTCACCTGCTCTTCCTAAGGGA
TGCTGGGTCTTGAGGAACCGGCAGTAGAGACACTGAACTGCTTATAGACTCACATCTGGTATCATCAGGTTGGACCTGGGATACTGTCTGGCTA
ACTAACACTTCCTGGCCCCCATGACTGCAGAGTTCCAGGTTCTACATCAGTACCCGGTTCTCTGAAATATGTACTCATTGGGGTCTCATGAATA
TGGAATTGAAATCCATTTTACTGTTGTACTCACTTAGAGGGAACATAGCCTTTGTTTCCTTACTTAACCATGTGAGCCACAGAGTAAGAAGAAA
ACCGTCTCTTTAAAAGCTGCTATTTAGGTAGCTGCCTTCATGGCAGATAAATCTTTTCCACAGGTTTGGAAAATATCAAAGATGCATCTGTTTC
ATAATTACATGGTGGCTTTCTACCTAATGAATGCCTGTGTAGGCTGCTACGTTAAGCATACAAAGAGGATAGGAGAAGAAAGTTACTTCGTATT
TATTTTTCAGGCTTTTCCTGTCTGTGTTCTCACTTCGTTATTGTGGGGTTTCTGCAGACTGAGTCCCTGTGGAAGAGAGGTTGTGTCCTGCTCA
TTGTGGTACAGCAGTGGCTTAAGGGAGACTGGAGCCCTGACTTCACCCCCACCCCACTCCCTTTATTTGACCTGGGATTAAAAAAAAAAAAAAA
GACAACTTGCATTTCAAGGTGAGTTAGCTTCTGATCTTAATATAACTTATTTTCCTTCAGGAAAAAAAATGCTAAAATCAAGACAGGGTACAAG
CGGGAGCACATTAACCTGGGCTGCGACATGGATTTCGACATTGCTGGGCCTTCCATCCGGGGTGCTCTGGTGCTAGGTTACGAGGGCTGGCTGG
CCGGCTACCAGATGAATTTTGAGACTGCAAAATCCCGAGTGACCCAGAGCAACTTTGCAGTTGGCTACAAGACTGATGAATTCCAGCTTCACAC
TAATGTGTAAGTGTACATGGGGGCAGCAGGGTGGGAGGGGTGGGGTTGGAGCATGTGTGGTAAGGGGATTGCTTTTCACTGTTTTTTTTTTTTT
TTTTTTAAGAAAGCCCAGTTTTTAACCACTGTAAAGTGAGTTTGTTGGCATTAGATGAGCCACATGGCCTGCTGCTATCTTTTGTTTCCCACCT
ACCCATTTCCTGGAGTGTCTTAGATCCTTTCCACTCTGTGAACAGTATATATTGCCACACAGGTACAAAGTGGCTGATTAATTACAGCAGGGAC
ATGGTGTCAGTCCAGAGCGTGTCCATTCCCTGCAGTTTTTGTTTTAGTTAACAAAACAGCTCTCTTTCTCTGTCTAATGCCTGAGGATTAGCCT
GCTGGGCTTGATGACATGTAGACATGGTAGACAAATTTAGGAGCTGATTATCTCAGTTACAAACTATCTTCTCTGTACAGTGAAGCATGTAAAA
GTAACTATTGGACAGTATGGTGAAGGGAAACAGTGATTATAGGTTAGGAGAGAGCCCCTTGAGAATTGAAGTAGATTGATGATTTAAGTTAAGG
GAAACACATTTCATTCATGAATCTTGTTTTATCCATAAACCTTGTTTTATTTAGTTTCATGAGGAATTAACCTGTGACAGGCACAAGCTGGGTG
CAGAGTTACCATGTTTATTTCTTTCCACTAGAGGTCACTAAAGAAGTAACTTCTTAAACTTAAGGCAAATCAGTGATATTAATGTGTAAACTAA
TATTTCCATTAATAAATTTTTTGAGGAATTTTTATCATTAATTTGGAATCTGTTACAGCTAAAAAGTCACAGGAAAACTATAGAGATGGACAAT
CTAGTGGTTGGCAGGGGGCGGGGAGGGTATGACTACAAAGCAGTGACTGAGGGAGTTTCGGGGAGGGAACTGTTCTATATTCTGACTGTCGTCA
TGGTTACATTAATCTGTGTACGTGTGTTAAAATTCGTAAAAATGTATACCAAGAAAATATCCATTTTGGCCGGGCGTCGTGGCTCACGCCTGTA
ATCCCAGCACTTTGGGAGGCCGAGGCGGGTGGATCACGAGGTCAGAAGTTCAAGACCAGCCTGGTCAAGAAGGTGAAACCCCATCTCTACTAAA
AATACAAAAATTAGCTGGGCGTGGTGGCGGGTGCCTGTAATCCCAGCTACTTGGGAGGCTGAAGCAGGAGAATCGCTTGAACCTGGGAGGTGGA
GTTTGCAATGAGCCGAGATCACGCCACTGCGCTCTAGCCTGAGCAACAGAGCAAGACTCCGTCTCAAAAAAAAAAAAAAAAAAAAAAAAAAGCA
AGAAAATATCAATTTTATTATATGTTAATTAAGAAGAGGCTGAAACATACTAAAAAGCTATAGGAAACATACCACATGCATAGAAATGTTTTTG
AACGTAGAATTCTGATTGGCATGGGAAGTGTTTTCCTTGGTTTGAAATTGGGCATTGGCACCAATTCTTCAGTACCAATTCTAGGAGGAACAAT
GGCTGTTAGCCATTTCCCCCACATATCTGAGCTTCAGCATTTTAAATAAGCAACAAGTGGGTATGGTTTATTTTTGGAACCAGCGTGAAGGCAG
CTGACACAACTCATCTGGGTTGCCCTGGTGCTTGCAGGGGCCCAAATGCATAACAGAAATTCTTTGTGCTTCATATAGATGAATTTGAACAGTT
CCACCTGTGTATTTATTTATTTATTATTATTATTTTTTGGTGACAGGGTCTCACTCTCTCACCCAGGCTGGAGTTCAGTGGCACGATCTCTGCA
GCCTCCACCTCCCGGGATTAAGTGATACTCCCACCTCAGCCTTGCAAGTAGCTGGGACTACAGGTGCATGCCACCACACCTGGCTAATTTTTGT
ATTTTTTGTGGAGGCAGGATTTCGCCATGTTGCCCAGGTTGTTCTTGAACTTGTGAGCTCAAGCGATCTGCCCGCCTTGGCCTCCCAAAGTGCT
GCCATTACAGGTGTGAGCCACTGCATCTGCCTTCTGTTTATGGTTTTGAAAGCTCTGTACAGGAAGGCAGTATTGTGTTCCTGGTTTTAGGATA
TTCTGAAAAGTCTGGCCTTTTTTAAAAACATGTAAGCCCAAGAAACTTCCCCATTTGAATATATTACTAAAGTCATTCAAAAATCTTATTTTTG
ACAGGCTGCTGTTTTATTGTAGCATACTTGGGCTGTCTACAGAAGACAGGATTCTCTGTGAATCAGGCAGTGCCTGGGTCTCCCTGTACTTTCT
AGAAGCATAATGTCTGTCTCGCTGAATTTCATGGGGAAACGAAAATTTACAATGTGTGGCTCATACGTGTAATCCTAGCTACTTGGGAGGCTGA
GGCGGGAGGATCACTTGAGGCCAGGAGTTTGGGACCAGCCTGGGGAACATAGAAATACTCTGTCTCTAAGAAATTTTTTAAAAATTAGCTGGGC
ATGGTGGCAACATGCCTGTGATCACTTGAGCCCAGGAGTTTGAGGTTACAGTGAGCTATGATTTTGCCACTGCTCTCCAGCCTGGGAAACAGAG
CAAGATCCCATCTTGGGGAAAAAAAAAAAAGAAAATTTAGAATGTATTCTCTGATTTAGGGAGAGTGTCTTGTGAAGACTAGAGTTTTTGTTCT
TAGGTCCTGAAGCCCTTTGCTGGATTCCAAAGTACGTGGGCTCTGACTCACAGTTAACCAATGCCATTTCCACTGGAAATGCCCACAATGATGA
GGCAGAACTCAGACACTGATTTTATGTTAAAGATGTTTGAGAGGGTCTAGGCAGGCTTGAAGACCATCTGATGCAAGGAGGATGGTCATTCCTT
TTTAAAAAATTGCAGCATTAAAATTTTTCTTAATTTTTTTAATCAAAGGAGTCTGAACCCATGGTTGTAAAATCAAACAGTACAGATGGGCCTA
TGAAAAGCAGTGATTCCAGCCTGCCTCAGCCTGCTCATCCCAGCTCCGCAGTGGCAGCCACTTTAACTGTTTTTGTTTTCTGTGGTTCCCTCCA
TGGAGAAGGTTGCTTCTGACCAGGCACCAGTATGCACTGTGTGTATAAACAACAATCTGAAAGCAGTAATGAGAGACAGTCTATGATCTGTGCC
GACCCCAGCCCGACAGAGGACAAAGTGTAGACTCTGAGCTCTTCGTATGCCTTTTTAGGGGTGGCAGTGACTGCAGAGTTTGTATAGGCTGTTC
AGCTGGGTGGGGCAGACTTTCAAATTCTGAGAGCTTAAGCAGGGCCCCAAAAGAGGAAGAGGAGAATTTGAATGACTGAAGAAGGGAAAGGGAT
ATTCCCAGCAACTGGAGTCATTCAGTGTGTTTGTAAGAGAGGAGGGAGCTTGGCCAGCTGGGGTTTCATTCTGAATGTTTTTGAAAGAGGGAGA
CATTTTATGATGTAGCAAAAAGCTCCCTTTCAAATCCTGCCAGGCTTAGATGTTGCAGCATGGGTGAGGAGTATGCACAGGTGGACGAGATGAA
TTGTGGATTAAGCTCTTTTGAAGTGCTGCTCCTCCAAGAGGCTGGGTCAGTGCCAACTGTATCTCCCCAGCAGTGGCTGCAGCAGGTTGTGCCA
AGGGCTCTAACTTGCCTTCTGAGGCATATGTGCAGCCCCACCTTTCTTGCCATTTCTGGCTGTGTTCTCTTCTCATTTTCTTCTCTTTCCATAA
CATTTTTTATTTTTACTTTTATTTTTTGAGCCAGAGTCTCACTCTGTCACCCAGACTGGAGTGCAGTGGTGAGATCTCAGCTCACTGCAACCTC
TGCCTCCTGGGTTCAGACGATTCTCCTGCCTCACCCCCCCAGGTTGCTGGGATTACAGGCACCTGCCACCATGCCCAGCTAATTTTTATGTTTT
TAGTAGAGACAGGGTTTCATCATGTTAGCCAGGCTGGTCTCAAACTCCTGACCTCAAATGATCTGCCCGCCTTGGCCTCCCAAAGTGCTGGGAT
TATAGGCATGAGCCACTGCACCCGGACTTCCATGCCATTTTTCTGGTAGAATCGTTTATAAAAGTAATAACATCAGTAGCAGTAAAAATAAATC
ACTCAGAATCTCAGTGCTCTACTTGCTGCTTCTTTTTTTTTTTTTTCCTTATAGAGACAGGATCTCGCTCTGTTGCCCAAGCTATATCCTGGGC
TCAAGCACTCCTGCTGCCTCGGCCTCCCAAAATGCTAGGATTACAGGCGTGAGTCACCACGTACCCCCTCTCCCCTGGCTTTTTCTTTTTTTGT
TTGAGACAATGTCTTGCTCTGTTGCCCAGGCTGGAGTGCAGTGGCGCGATCTCGGCTTACTGCAACCTCTGCCTCCCAGGTTCAAGCGATTCTC
CTGCCTCAGCCTCTTGAGTAGCTCGGATTACAGGCGTATGGCACCAGCCCGGTCAATTTTTGTATTTTTAGTAGAAACAGGGTTTCACCATGTT
CGCCAGGCTGGTCTCAAACCCCTGACCTCAAGTGATCTGCCCATCTCGGCCTCCCAAAGTGCTGGGATTACAGGTGTGAGCCACTGTGCCTGGC
TAGGTGCCTGTTCCATACTTATCAGACTTCCTGTAATTTAGTACCTGTTTTGTGAAATTTGGTGAACTTCTGACTTCCCTTAATACCAGTATTG
GTTCAGTCCATTTCTGTCATCCCTGTCTTCTGGGAGACTTGGGTGGATCTGTCTGGGCTGCAGCTCACCTCCCTGGGAAGCTCAGCATCTGTCC
TGTGCTTGTTTGCAGGAATGACGGGACAGAGTTTGGCGGCTCCATTTACCAGAAAGTGAACAAGAAGTTGGAGACCGCTGTCAATCTTGCCTGG
ACAGCAGGAAACAGTAACACGCGCTTCGGAATAGCAGCCAAGTATCAGATTGACCCTGACGCCTGCTTCTCGGTGGGTATCTGTGGAATCACGC
ATCTCACAGGCAGGCCCATCTGCTCTTTATGTTGGAATCCTTCAGGTTTGCTGGAGTGCTGTGCTTCAGCTTACTCAAAGTGAGAGATGCTGCT
GGGCGCCGTGACTCACGCCTGTAATCCCTGCACTTTGGGAGGCCAAGGCAGGCAGATCACTTGAGGTCAGGAGTCTGACACCAGCCTCGCCAAC
ACGGTGAAACCCTGTCTACTAAAAGTACAAAAATTAGCCGGGCATGGTGGCGTGTGCCTGTAATCCCAGCTACTCGAGAGGCTGAGGCAGGAGA
ATTGCTTGAACCGGGGAGGCGGAGGTTGCAGTGAGCTGAGATCGTGTCACTGCACTCCAGCCTGGGTGACAGAGTGAGATTCTATCTCAAAAAA
AAAAAAAAGACTCCGTCTCAAAAAAACAAAAACAAAGTGAGAGATGTTCTCACTTTGAAAAGAGTGCCAAATGAGTACAGTGATCATACTACTA
GTACTTCTTATGGGTACCAGGCACTGTGCAGAGCCTTTTGTGTAGATTACTGTGTTTAATCCCATTTGAAATAGGTGCTTTGGTTTTTTAATCA
TCTCTGGGTTTTTTTTATTTTTTGTTTTTTTGAGACAAGGCCCAGGTTGGATGCAGTGGTGTAATTGCAGCTCACTGCAGCCTCATCCTCCTGT
GCTCAAGTAATCCTGCCACCTCAGCCTCCCAAGTAGCTGGGACTATAGGCACACCAGCATGCCTGGCTAATTAAAAAAAAATTTTTAGTAGAGA
CAATGTCTCACTCTGTTGCCTGGGCTGGTCTCAAACTCCTGGGCTCAAGCTGTCCTCCCACCTCAACCTCCCAAATGTTGGGATTACAAGGCAT
GAGCCACCATGCCTGGCCAATCATCTCTGTTTGTTTGTTTTTTTTTTTGAGACGGAGTCTCCCTTTGTTGCCCAGGCTGGAGTGCAATGGCACG
ATCTCAGCTCACTGCGCAACCTCCGTCCCCCAGGTTCAAGTGATTCTACTGCCTCACCCTCCCATGTAGCTGGGAACACAGGCACCCACCACCA
CACCTGGATAATTTTTGTGTTTTTAGTATAGACGGGGTTTCACCATGTTGGTCAGGCTGGTCTCGAACTCCTGACCTCAGGTGATCTGCCTGCC
TCGGCCTCCCAAAATGCTGGAATTACAGGCATGAGCCATGGCCCCTGGCCAATCATCTCTGTTTTTACAAATGAGGAACTAGAAACTTAGGTGA
GTAACTTGCTCAAAGGTACACAGCAGGAAAGTGCTGGGAGCTGGATTTGAACCAGACTGGCTTACTGTTAAGCCTCTTCCATTAATACCAGTTC
CTGTTCTATCATGCGGACTGTTAAAAGGGGCACTCTGAAATCTAGGGTTCTAATTGATAATTCCCCCCGCCCCGCCTCACTTTTCATTCTCCTT
TTCAGCTCCTGCATTGCATATTAAGAAATCAATAGTCAGTTGGCATCTTGCAGAGTCTCTGGCATTGCTCACAGGCCCTCGAACTACTACACAG
TTTTTGCAGTAAATCCTATTGGGCTCATTTCTGTTGTCACAGGGCACTGGCGGCTGTATCTCTTTGGGGTGAAATGAGGGAATCTCATGTTGCA
GAATGGTGTCTGTACTGGTTTGTGTAATTTATGGACAGCCCTTTTTAAGTCTCCTAAACTAGAACCGAATTTGGCTCCCTTAACAGCAAGCACT
TAAAAACAAGAGAAAAGGAGCCTCATTATTTATATGAGCAAAAGGACACTGGCCCCTAAGAGGCCTTCCTTCTCTCTGCTTTGTTCTTATGAGG
GGTGTCAGATCTCAGCACACCCTTTGAACAGAAAGGTATGCACCTAAAATTGATGTATTTTGTATTGAATTTAATGAATCTCATGAACAAGGTG
GTCATGAAAAATTTTGATTCTAAAGTCCAAGGTTGGTTGACTAAAAAGCTCTAATTTTGGAGATGGAAGTGCAAAGTATTATACTTAATGTTTT
CTCTGTTTTGTCATTTCCTTGATTAGGCTAAAGTGAACAACTCCAGCCTGATAGGTTTAGGATACACTCAGACTCTAAAGCCAGGTATGTGTTG
CTTGTGTGTGAAATCGGTTCTTTATCTTAAAAAAATTGTGCTGGTGGTTTGCTGATTTATGTCATTGCTTATAAAATGTTTATATAATACAGTG
GGTATATATAAATGGATATATGTAAAGTATAATTAAATGTTTATAAAGGAGTATGGGGTCATTATAAAACATTTGGAGAATATGAGATGTAGCA
AGCAGGAGGAAAAAGGCTGAGGCTAACACATATTCTTTGCTATTTTCTTCCATCCTTTTTTCCAGTGTGGACATAATGCTTGATACTTATTTCT
GGCAGATTCTCAAAACTAACTTATAAATCAGCCCCACTGTAAAAAGACAAAGAAATAGGAAAAGTGGTTTGCAGGTTTCCTCTGCACAGTTGGC
TAGGCCAGTCCACGGCGTATTATAGTTCTAGTGTATAACCACTTTTGAACCCTGGAAATCTAAGACTACCAGCTACTCCCTGAATCAGGTTATT
CCAGGGAATGTCAGTTTGTTGTGTGCATCTGTGGATGTACATGCACACTACACATGTGTGTACACTATGAACATGAACATGTAAGACCTTAGGT
TGGATTCAGGATTGAGGTTTCATTTAAAGGGATGATTGCCAAAGAAGCTTACTGCATGCTACCAGTGCTATAGACTAATTGTACCAGGGGAACA
GTGAGAGGAAAAAGGAAAGTGGGGTTCCAGAAGTAGATTTGGGTAGGCTCTCTGGGCGGTGGGATAATTATAGTTGTTTTATAAGCCCACTGTA
GTATGCCTGTTGGTCAGATAGTGCTAAGTAACAAGGATTGAAGGGCCATGTAGTTTGGACCCTGCTGATAATACCTGGAACAGGTTCTGAATTT
GGTCTTAATCTTTCTTGTCATTTCTTTCCTCCATTCCTCCTCCTTTCCTCTCCCTCATTTTTCTCCCTGCAGGTATTAAACTGACACTCTCAGC
TCTTCTGGATGGCAAGAACGTCAATGCTGGTGGCCACAAGCTTGGTCTAGGACTGGAATTTCAAGCATAAATGAATACTGTACAATTGTTTAAT
TTTAAACTATTTTGCAGCATAGCTACCTTCAGAATTTAGTGTATCTTTTAATGTTGTATGTCTGGGATGCAAGTATTGCTAAATATGTTAGCCC
TCCAGGTTAAAGTTGATTCAGCTTTAAGATGTTACCCTTCCAGAGGTACAGAAGAAACCTATTTCCAAAAAAGGTCCTTTCAGTGGTAGACTCG
GGGAGAACTTGGTGGCCCCTTTGAGATGCCAGGTTTCTTTTTTATCTAGAAATGGCTGCAAGTGGAAGCGGATAATATGTAGGCACTTTGTAAA
TTCATATTGAGTAAATGAATGAAATTGTGATTTCCTGAGAATCGAACCTTGGTTCCCTAACCCTAATTGATGAGAGGCTCGCTGCTTGATGGTG
TGTACAAACTCACCTGAATGGGACTTTTTTAGACAGATCTTCATGACCTGTTCCCACCCCAGTTCATCATCATCTCTTTTACACCAAAAGGTCT
GCAGGGTGTGGTAACTGTTTCTTTTGTGCCATTTTGGGGTGGAGAAGGTGGATGTGATGAAGCCAATAATTCAGGACTTATTCCTTCTTGTGTT
GTGTTTTTTTTTTGCCCTTGCACCAGAGTATCAAATAGCTTCCAGGAGCTCCAGCTATAAGCTTGGAAGTGTCTGTGTGATTGTAATCACATGG
TGACAACACTCAGAATCTAAATTGGACTTCTGTTGTATTCTCACCACTCAATTTGTTTTTTAGCAGTTTAATGGGTACATTTTAGAGTCTTCCA
TTTTGTTGGAATTAGATCCTCCCCTTCAAATGCTGTAATTAACAACACTTAAAAAACTTGAATAAAATATTGAAACCTCATCCTTCTTCTGTTG
TCTTTATTAATAAAATATAAATAAACAGGGCATTTGTTTAGATTTATTTTGTCTTCATGCTGTGTGTATTATATATTATTCGTTTTCACTCACT
GTACAACAGATTACCATATGTTTAGCAGCTTAAAACAACACTCATTTATTTCACAGTTCTGCAGGTCAAAGCGAGGCAGGTGTGCATGCCTACC
TGGACTGTGCTTAGCTTCTCACAATGCTGGAATTAGGGTGTCGACCAGGCTGTCTTCTCATTTTATCTGGGGGCTTGGCGTCCTCTTCCAGGCT
TTCAGGGTTGTGGTGGAATTCAGTTATTTTCAGTTGTAGGACAGAGGTCCCTATTTCTTTGTTGGCTGAGCCACTCTCAGTGCTCAGAGGATGC
CAGCATTCCTTGCCATGGGGTCCCCTCCATCGCCAAAGCCAGAAACAGAAAAACTCCCTCATGTGAAATCCCTCTCACACTTCCAGTCTCCTCC
AGGTAGAGCCCCAACCCTTTTAAGGACTCACCTGATTAGGTTAGGCCCAATGAGGGTAATCTCCCTTCAAGTTAACTGCCAGGTAAACTAACCT
AATCACTGGTAATATCCTTTCATGTTTGTAGGTCCCTCCATATGGGTCATGGGAAGGGGTGATTTTGGAATTCTCCCATATATTTATATGTTTT
TAATAACTTGATGGTGGATTTTTTTTTTTTTGAGGCGGAGTTTCATTCTTGTTGCCCAGGCTGGAATGCAATGGCGCACAATGTCTGCTCACTG
CAAGCTCCGCCTCCTGGGTTCAAGCAATTCTCCTGCCTCAGCCTCCCCGCCTCCTGGGTTCAAGCAATTCTCCTGCCTCAGCCTCCCGAGTAGC
TAGGATTACAAACATTTGCCACCACACCCGGCTAATTTTGTATTTTTAGTAGAGATGGGGTTTCTCCATGTTGGTCAGGCTGGTCTCAAACTCC
TGACCTCAGGTGATCTGCTCACCTTGGCCTCCAAAAGTCCTGGGATTACAGGCGTGAGCCACCACATCCAGCCTATTGTGGAAACACAAATAGT
AATAAACTCTATGCATGATCCAGCTTCAACAAATATGAACATTTTTTTCCATTCATGATTTTTCAGCAGAATTCCTTTTTTTTCTATGTTTGAA
AGTGACATTCAAAACAGGAACCATCTATTTTAGCATTTTATGCAACTGATCACCCAAAAACTGTGGGCCTCAGTATTTGTCATACTGTCTTCAG
GGACTTGCAGCTATTTCTAAGTGAATGTTCATAGAATTTATCCAGAGATTATGGCACATAGTGGTTGCTGGGTACATCTAGTCAGATGTCAGGT
ACAGTATTAAGATATTTTACTTTTAGAGACAGTCTTACTCTGTCACCCAGACTGAAGTGCACTGTCATCATCATAGCTCACTGCAGCCTTGAAC
TCCTGGGCTCCTCTGATCCTCCCATCTCAGCCTCCCGAGTACTTGGGATTACAGGCATAACCCTGCCCCTAAAATATTTAAAAATGTATAAAGG
GTTCTCTTGAGTATGAGGGAATATTTCCCTCACTAGTTACTTATTGTCTGCTTGTTGCATGCAGCATGTTGGAATATCATTGTGCAAAATGCTT
TGAGTTCATAATATCTCACCAGTTAAGAGAATTGAGACAATCTGGTATGAGGACACAAATGGACAAAGTGGACAATGAGTCAGGGAAGAGGACT
GCAGTGTGTTTATTATAGATTAACTTTAAAGTCAACAAGCAGAAACTAAGATTGAGAAGCTCATTTGTTGAACTGAGTGGTTTTACAGAAAACT
TAAGTACCGCTCAGAGAAGGGAAGCTCCTGGGGGACTGGGAGGACTTTGCACCTTGGCTAAAGTGGAGAGTTTGCAATAAGGACACCATTCAGT
CTAATACGGGCTTCTGGGGGATTTCGGGAAATGTGCAGATGAATGACTTCTTAGTTTTCTTAACACAGAGTTTGTGTGTTTCTGAGCTGTCACC
AGGGTGAGATAAAAGCTTAGGGGAAACCAGATTCAGGAGTAGAACACAGGCCCTTTAGAGAGACAGCTCAGCTGTGTCTCTGGGGGATAACAGC
AATCTTCGCAGGGAATCATAGAGACCAGAATGGTGAAGCTGTGGGCACAATAAATGTCTTGCTTAACGTCACATTGATTTTCCTGAAATTGATC
AAATGGAACTTTTATGAATTGAAGCCTGTATCTGTTTATTAAGGAGACATTTACAGAACGTACACACAGAGCAAAGTATGGCGCCAGGGCCCTA
GGTGAACTTCTTGGAAGTTTGTAAGTTTGCATTTTTATACACTGGGGATTTCAAGTAACAAAATTTAAAAAGAGAAGCTTATTTTAGGAAATCT
GAAAGTTATTTTCAATGGCAGCCCCTCACCTCTACTTCCTAGCTACCTTCCTTTCTTTTCCTCCATAATTTTTTTTTTTTTTTTGAGACGGAGT
CTGTGGCCCAGGCTAGAGTGCAGTGGCATCTCGGCTCCCTGCAGCCTCCACCTCCCGGGTTACAGCGATTCTCCTACCTCAGCCTCCCGAGTAG
CTGCGATTACAGGCACGCGCCACCACGCCTGGCTAATTTTTGTATTTTTAGTAGAGTCGCCGTTTCTCCATGTTGGCCAGGCTGGTCTCCAACT
CCGGACCTCAGGTGATCTGCCCTCCTCGGCCTCCCAAAGTGCTGGGAGCCACTGCACTAGGGCCGGTCCATAATATCTTTATCATCTTGTATAG
TGTGTATTTTACACCATATTCGCCCTCACAAGAAAATAAACTGCCAAGGCGGGCGGGGGGGGGGGGGTTCTTTTTGTTCACTGCTGTATCTTAA
ATAGGGAGAGGTGCTAAATCTTTGCTGAAAAAAAATAAATGACTGTCGGGAAAGATTTTTAAAAATTACTTATAATCCCATCACCGAAATAGGA
CTACTTAACATTTGTGTTTCTTTCGCCTTTGAGAGAGGAAGAGCAGTTACTTCGCTTTCGTGATCAGTCTGTTCTACAGCTGGATCTTCCTTTT
TAAAATGGCTAAAAACGCTATTTTTGGCTGTATAGTACCCAATCATGCAGGCGGCAGGGAGGGGCTCGCTTTGGCCCTCTTCAGCACGTCACAC
CCTACCTTTAATCCGCAGCCAACTGTGTGGCGGTCCCCACTTTCGCCTAGCGGCAACTCCCTCCTCCCTGCAATCCTTCCTTCTTATTCCGGTT
TCCAGAGCACGTCTCCAGGCTTCGACGTCACCACGCTGGGACGTATGTGCCGAGGGCCGCGGCGTCTGACCTCATGGCGTAGAGCCTAGCAACA
GCGCAGGCTCCCAGCCGAGTCCGTTATGGCCGCTGCCGTCCCGAAGAGGATGAGGGGGCCAGCACAAGCGAAACTGCTGCCCGGGTCGGCCATC
CAAGCCCTTGTGGGGTTGGCGCGGCCGCTGGTCTTGGCGCTCCTGCTTGTGTCCGCCGCTCTATCCAGTGGTAAAGGGGGGCGGCAGTGTGGGC
TTAGGAAGGCTAGGAGGGCTCGGGCGCAGGGCAGGAGACAGGCCGGGCCAGGGAAAGGGCGCTTCCAAGTGTCAGGAGCAGACGGCGGTGTCTG
GTTGGGTCAGGGTCTCCGCGGGCCTGAGGACGGAGGGTATGGTGTCTGAAGGGGTCCAAGGTGGTAGGGTCTCCAAGGGGCCTGAGAAGCGGGG
TCCCTGAGAGATGCAGGAACCCAGGGAGCAGGCAGCAGGGTGTGTAGATGGGCGGGGAGGGAGGAGTGGTGTTAGCGCCTAGAGATCGTTGAAG
AATGGGCTGCCGCAAGGAATGGCGGTGGTGGGGTCTCCAAAATTTCTTGGATCGGAAAGTTAGGAGACTCTGGAAGAGAGGGTGGCGACAGGGG
CTGAGGAGGTGGCCGGGGCTGGGGGTGTGGTGTCTGGAAAGGGTCCTGAGTTTGGGGGACTCAGGAATCTTGCAGGACCAAGGAAAGGTGGGGG
TTTTCCAGAATGGGAGAGGTCAGTGGTAAAGACCGAGGCGGGCAGGGGCAGTAATCCAGTGACTGGACTGAACCAATGGAATATTTCCGAGAGG
GAAAGTGAAAGGGAAAGAGCTGTCTTAGGGTACTGGTGCATATTTGAAGACTGGGAGGTTCTGGTGTTTGATAAAGATTCAGAGTACTGGGGCG
TTGGGCCCCTGAAGGCTTGGGAGGCAGAGAGGAGTTTGGAGACAAGGGAGATCATGATTCCTCTTCCCTGTGTTCCGAGTTAACCAGGATAACC
CTGCCTCGTTTTAATGCATCTGTAGGATTTGATTGATAAAATATTTCATTTCTTGCAAATGTTGCATGGTGAAATCAAGAGATTGAAGGCTTTT
ATTGAGAAATTAGGAAATCTGTAAACCAGTGCCTGATACTTGGTGGGAAAAATCCACTATTAAGAACTTGTAAGGATTAAACGGTATTTTGTTT
AGTAGTCCCATTTTTCTTTCAGGCCAAGACACAGTCCTGCGTTTCTCCCTAAATTTGAATTTTCAGCTGCAAAACAGTGTGACCTGGTATAGTG
TTTTCTTTCTTGCCAACAGGGCCTGTCCAGCCCTACATTCCTCTGCGACAGTACTGTGGGTACATACTTATGTTTCCTCAGCAACATTTCAGTG
TTGCTAACTGGACGCCCTCCCAGAAGTCTTTGCAGTCCCTGAGGAAGGACTGCTGTCCATTTCCTAAGGCCCTTTTATCATTAGTGAAATGTAA
TGAGGATTTCCCTGGTTGATGCAGATATTCCCGTTCCTAAAGTACTTAAGTAGCTTAATTGTTTTGGCCATTTAATCATCTAAGAAAAATAAAA
CACAAACATTTATATTTCCTGCTGGAATGCTTAAGGGATATATATCTGGCAACTGTAGTTTCAGAAACAGCTTTTTTCCCCTAAGAGATGGTTG
AAAATTGGGCAAGAAGTGAGGCAGAATGTGGACTGTTAAGTGTCCTTTAGTCTCTGCTTTTATTTGTTAGAGCCCTATAAATATTTTTCTAACT
ATACTGAGACATTTTAGAGAATCTTTATCTTTTAAATTAAGTCTTTACATACACCAGTTTAAAATGTCATAAATTCAGATTATTCTGTCCTGAA
AAGAAGCTAAATGAAAACGAACATACATGTCTTTAATAGGCGAGCAGCTCCACTGCACTACAGTGTAGCTTGGGATATAAGTATTTTTTGTAAG
CATACCTTATAATTTCAAAGTAGTCACTCCTGTAATAAGAAAATTAGTCTAGAATGTGTTGAGTTCCAAATTTCAAGTGAGTTTACAAAGGTAG
AAGGTTACCACATTAGATTTATTTAAATTTGTTCCATATTAGGTTTTAAAAACTGTCAATGCTTGGTTATATGCTCGTCAGTCTGTAAGAATTT
TTGTTTTATGTTTTTGTTTTGATCAAGTCTAAAAAATTAACATAAGCAGTTTGGATCACTGGGTGACTAATTCTTGGTCATGCCATTTCAAAAA
TGCCTGGTTCTCTACATTTGCCATTATATTGTCCCCAAATGATCACCTAAACTTAATGTATAATGCATTTCATTTTTAAGGCATTGTTTCTCAA
ACCAGTGTGTGAATGAGTTCTCGGGGTCCTCTGCATTCTTAAGTTTAAGAAATGCTGATATAGTTTTATTTTAGGGCAGTTTTTAAAAAGGGAC
CAGTAGAAGGGGAGAATTAAATTTATAAAGTTTTGTTTTTTGTTTTTTTTTTGAGTCTGTCGCGAGGCTGGAGTGCAGTGGCGTGATCTCGGCT
CATTGCAACCTCCGCCTCCCGGTTTCAAGTGATTCTTCTGCCTCAGCCTCCCGAGTATGCTGGGACTACAGGCGCGCACCACACCTGGCTAGTT
TTTGTATTTTTAGTAGAGACGGGGTTTCACCGTGTTATCCAGGATGGTCTCAATCTCCTGACCTCGTGATCCGCCCGCCTTGGCCTCCCAAAGT
GCTGGGATTACAGGCCTGAGCCACTGTGCCCAGCCTTTTTTGTTTGTTTGTTTGAGACAGGGTTTTGCTCTGTCGCCCAGCCTGGAGTGCAGTA
GCACAAACTTGGCCCATTGCAACTTCCGCCTCCTGGGCTCAAGCAGTCCTCCCACTTCAGCCCCCTGAATAGCTGGGACCACAGGTGCCTGCCA
CCGTGCTCAGCTGATTTTTGTATTTTTGTAGAGATGGGGTTTTGCCATGTTGGCCAGGCTGGTCTTGAATTCCTGTGCTTAAGCGATCCACTTG
CCCTGGCCTCCCAAAGTGCTAGGGTTACAGGCGTGAGCCACCGTGTCCGGCCTAAGCTTTATTTTTAAAATGTTAAGCTTCAAGTAGAGAGGTA
ATATCAATTAGGAAAGGGAAGATTTAGAAGTCTTATTTATATGCTCATAGAAGGTCAACAGTTAAATCCGTAAAGTCAGAGTTGTACCTTTGTT
CTGAATACTCTTCATATCTGACTTAATCAACTTCTCTGATTTGAAGGAGTTTGAGTAAGTTGATAGCCTTTGTATTACATGTTTCAGGTCAATT
ATTTGTTTTTTCTTGTGCTGTATTTGTGTATTACTTCCAAGATTTTACTGCTTGACTAAGACATTCTGCTCCTACTTGGTGCTAGGTATTGGAG
TTAAACAATGAATAGGGTGATACTGGGGGTATCCTTAGAAGAATGAGTGGCGTTTTGCTGAGAACCATGGCATGGGCTCTTGAGGGAGTGCAGT
AGGTGCATAGATGCTATGTGGGATGTGGTGTGGGATTTGTGGTGAGAGTTGAATCGAGAGCATATTCAATAAATATTTATTGAGTGCCTGCTAT
CTATTAAGTAGGTAATGGGGATAGAGCTGTTAGGGAGATTAGTAAGATAATGAGAGTGAGGTAATGGGGGCCAGATCATGTAGGATCTTGAAGG
CTATTATAAAAATTTTCACTTTTGTGAGAAATGGATGGCCCTAGAGGGTCTTAAGTAGAGGAGTAACATTTGATTTAAGTTTTGACAGAATGAC
TCTGGCTGCTGTGTTGAGTATAGACAGTAGAAGTTAGGGTGGAAGCAGGCAGATTAATTAGGAGGCTATTGTAATAATCCATGTGAGATAATGG
TTACTTGGATGAGGCAGGAGCAGTGGAGGGTGAAAAGCCCTCAAATGCTGCATATGTCAGAATAACAGATTGGATGTGGGATGTTAAAAAAAAT
GACAACTCCAAGGTTTTTAGTTTTAGTAACTACAAAGATGTAGTTGCCATTAATTGAGATGGAGACTAAGTAGAACAAGTTTTTTTGTTTTTTG
AGTGGGGCATAGGAATTTATTTAAACTTATTTTGAGATAATTATAGATTCACTTGGCAGTTGTAAGAAATAATATAGAGATCCTGCATATCCCC
TACCCAGTTTTCCTAATAGTAACGTCTTTCAAAACAATAGTGCAATATCACGACCAGGATATTAACATTGGTACAGTCAAAATACCGGGCATTA
CCATCACCACAAGGATATCTCATATTGCCCTTATAAAGCCACCCCACTTCCCTCCTACCTCACCCCTCCTGAACTCCTGGTAACCACAAATTAT
CCATTTGTATAAGTTGGTAATTTTGAGAATCTTACATAAATGGAATAATACAGTATGTAACTTTTGGGATTGACTTTTTTTCACTTAGCATAAT
TCTCTGAAGATTCACCTAGGTTGTTACATGTGTCAATAGTTTGTTCCTTTTTAAATTTTAACTTTTATTTTTTCGAGAGTAAGTCTTGCACTCC
AGGCTGGAGTGCAATGGCACGATCTCGGCTCACTGCAACCTCCGCCTCCCAGGTTCAAGCAGTTCTCCTGTCTCAGCCTCCAGAGTAGCTGGGA
TTGTAGGTGCACACCACCACACCCAGCTAATTTTTGTATTTTTGGTAGAGATGGGGTTTCACCATTTTGGCCAGGCTGGTCTCGAACTCCTGAC
CTTGTGATCCACCTGCCTTGGCCTCCCAAAGTGCCCAGATTACAGGTGTGAGCCACCAGTCCTGGTTGTTTGTTCCTTTAAATTGCTGAGTAAT
ATTCTATGGTATAGGTAGACTACACTTTATTTATCTGTTGAAGGATGCCTGATTGTTTCCAGTTTTTGGCTATTATGAATAAAGCTGCTATGAA
TGTGTTTGTGCAGCACTCCAGCCTAAGTGACAAAGCGAGACCCTGTCTCAAAAAGAAAAAAATAAAATGAGCTTTCTATGGACATCATGTAGTT
GAGTCTGCCAGTCTCTCTTTTTTAATTGATGTATTTAGGTCATTTACATTTACTGCACTATGGATATGTTAGAGCTGAAGTCTGCCATTTTTAT
TTTTTGTCTTTAGCCTGGGATATATGAGGTAAAAAGAAAAACCTAAGGAAGTCACCACCATGTTGTTTCACATATCCTGAGGTCTCTAACTGTT
CTACCCAGTTCTTTCCACCTTTCAGAGTGTTCTTGTGTTATTTTATACATAATGTTCAGAGCTTTTAGTTGTTTTTAGTGGGAGAAACAGGGAA
GAGTGTGTTTGTCTTCCTGAAAGCCAGCCAGAATTCATTTTGAACATATTAATGTGAATGTCAGACATCTAAGAGGAGATGTCAAGTAGGTGGT
TGGATTTATGAGTCTAGAATTCTGGTCGTTCTTGGAAGTTCTTTTAATTTTTCTCAGAAAAGTTAGGAAGTGATGTCATCAGTTGAAAATGAGG
ATAACAGAGAAGACATTTGAAGTTTGTGGTTAGAGGAAAATGTGATTGGTGTCCTAAGAGAGTAGAAGGGTAAATGGACTAGGAAAATATGATT
GCCAACAATTTTAATTGCCAACAGCTTTAAACATACACCTGAATCTTTGGCATTGAATTTAAAATGAAATGAAAACAGTGGCATGGTTGTATAT
TTTTTCTAGCCATGTTCAGCTGCTGGGTGCAGATGTGAGTAGTCAGAGAGCTTGATTTATTAATAACTAGGGATGAGTGTAGTCTCACCAAGTG
AATGGGTCAAAGTGGACAAGGGGGAAGGGAATTGAAGATGGATGTAAGAGAGTGATTATAAGCAATTATAATAGCTGACACAGAATTGAATCTG
AATAAAGAGAGAAGAGAGTGTATTGGGCAAGTTTTGATGGTGACCGTAAGGGAATGAAAAGGATTAGAGGGTTGTAGGTGTTTATGGATTGAGA
ATACCAGGGAATGAACTGGAAGGATAGGAGGTCTTGCCAGCAAGTAGAATGCATGAAATTGAGATTATGGAAGAATTGTGATAATAAATAATGA
CTCTGGGAGTGAGTGGCTGAGGTAAAGAGGAGGGCTAGCTCATGAGAAGAGAGGAGGTCAAAGATTTTGAAATCAAGGCAGGAGTACTTGTCTT
AGATGGAGTGAACTCTAGAGCCAGGAGCTAAAGATCATTAAGAATTAGGGGGCATGACTTTGGAGTTGGATGAAAGTAAGTTAGTGCATTATAG
CTTAAGATAAGAGAATCAAAGCTAGAGGCATTTAGGGAGAAGAACGGGAGAATGGTCTGAAAAGGGCAATGAGGAGGAGAACTGTGCCTCCTTG
AGATTCAGTGGTTCAAGGATGTGGGAGAAAAAACAGCTATTACTTAAACAGGCTGAAAGGAAAATGTGTCCTTAAGGGAGAGTCAGGTTTCAGT
AATAACAAGAAAGCAAAGGGTGAAAGAAGGGATTTGCTGATGACTGTGAATTCTATACTGCGTGGTGGAAATTTCTTTATAATTATTCCCACCC
TATCAGCTTGTGTCCTTTAAAGGTACTTACCCATGGAAAAATTGTTCTTTTAAAAGTTAAGTTTAGTTAGTTGGCTCCCACTTATTTCAATTTT
AAATTTTATTTTAAAACATGTTTTTTGTACTTTGGCAGGAAAATTAAATGTATCAGAAAATAATAATTTTTAAATTCTTGAATTATAACTAAGT
TGAAGTGATAAATGCAAAAATTATTTTCAGAAATTCCTGTTAGTAGTTTTCTGAGTAATGTACTGAGTTGTAGGTCCTCTTTATTTCTCTTTTC
TTCCAGTAAAAAATTTCAAGG
HUMAN SEQUENCE—mRNA
GCCGCTCGCTCGGCTCCGCTCCCTGGCTCGGCTCCCTGCCTCCGCGTCGCAGCCCCCGCCGTAGCCGCCTCCGAGCCCGCCGCCACATCCTCTG
AGAAGATGGCTGTGCCACCCACGTATGCCGATCTTGGCAAATCTGCCAGGGATGTCTTCACCAAGGGCTATGGATTTGGCTTAATAAAGCTTGA
TTTGAAAACAAAATCTGAGAATGGATTGGAATTTACAAGCTCAGGCTCAGCCAACACTGAGACCACCAAAGTGACGGGCAGTCTGGAAACCAAG
TACAGATGGACTGAGTACGGCCTGACGTTTACAGAGAAATGGAATACCGACAATACACTAGGCACCGAGATTACTGTGGAAGATCAGCTTGCAC
GTGGACTGAAGCTGACCTTCGATTCATCCTTCTCACCTAACACTGGGAAAAAAAATGCTAAAATCAAGACAGGGTACAAGCGGGAGCACATTAA
CCTGGGCTGCGACATGGATTTCGACATTGCTGGGCCTTCCATCCGGGGTGCTCTGGTGCTAGGTTACGAGGGCTGGCTGGCCGGCTACCAGATG
AATTTTGAGACTGCAAAATCCCGAGTGACCCAGAGCAACTTTGCAGTTGGCTACAAGACTGATGAATTCCAGCTTCACACTAATGTGAATGACG
GGACAGAGTTTGGCGGCTCCATTTACCAGAAAGTGAACAAGAAGTTGGAGACCGCTGTCAATCTTGCCTGGACAGCAGGAAACAGTAACACGCG
CTTCGGAATAGCAGCCAAGTATCAGATTGACCCTGACGCCTGCTTCTCGGCTAAAGTGAACAACTCCAGCCTGATAGGTTTAGGATACACTCAG
ACTCTAAAGCCAGGTATTAAACTGACACTGTCAGCTCTTCTGGATGGCAAGAACGTCAATGCTGGTGGCCACAAGCTTGGTCTAGGACTGGAAT
TTCAAGCATAAATGAATACTGTACAATTGTTTAATTTTAAACTATTTTGCAGCATAGCTACCTTCAGAATTTAGTGTATCTTTTAATGTTGTAT
GTCTGGGATGCAAGTATTGCTAAATATGTTAGCCCTCCAGGTTAAAGTTGATTCAGCTTTAAGATGTTACCCTTCCAGAGGTACAGAAGAAACC
TATTTCCAAAAAAGGTCCTTTCAGTGGTAGACTCGGGGAGAACTTGGTGGCCCCTTTGAGATGCCAGGTTTCTTTTTTATCTAGAAATGGCTGC
AAGTGGAAGCGGATAATATGTAGGCACTTTGTAAATTCATATTGAGTAAATGAATGAAATTGTGATTTCCTGAGAATCGAACCTTGGTTCCCTA
ACCCTAATTGATGAGAGGCTCGCTGCTTGATGGTGTGTACAAACTCACCTGAATGGGACTTTTTTAGACAGATCTTCATGACCTGTTCCCACCC
CAGTTCATCATCATCTCTTTTACACCAAAAGGTCTGCAGGGTGTGGTAACTGTTTCTTTTGTGCCATTTTGGGGTGGAGAAGGTGGATGTGATG
AAGCCAATAATTCAGGACTTATTCCTTCTTGTGTTGTGTTTTTTTTTGGCCCTTGCACCAGAGTATGAAATAGCTTCCAGGAGCTCCAGCTATA
AGCTTGGAAGTGTCTGTGTGATTGTAATCACATGGTGACAACACTCAGAATCTAAATTGGACTTCTGTTGTATTCTCACCACTCAATTTGTTTT
TTAGCAGTTTAATGGGTACATTTTAGAGTCTTCCATTTTGTTGGAATTAGATCCTCCCCTTCAAATGCTGTAATTAACAACACTTAAAAAACTT
GAATAAAATATTGAAACCTC
HUMAN SEQUENCE—CODING
ATGGCTGTGCCACCCACGTATGCCGATCTTGGCAAATCTGCCAGGGATGTCTTCACCAAGGGCTATGGATTTGGCTTAATAAAGCTTGATTTGA
AAACAAAATCTGAGAATGGATTGGAATTTACAAGCTCAGGCTCAGCCAACACTGAGACCACCAAAGTGACGGGCAGTCTGGAAACCAAGTACAG
ATGGACTGAGTACGGCCTGACGTTTACAGAGAAATGGAATACCGACAATACACTAGGCACCGAGATTACTGTGGAAGATCAGCTTGCACGTGGA
CTGAAGCTGACCTTCGATTCATCCTTCTCACCTAACACTGGGAAAAAAAATGCTAAAATCAAGACAGGGTACAAGCGGGAGCACATTAACCTGG
GCTGCGACATGGATTTCGACATTGCTGGGCCTTCCATCCGGGGTGCTCTGGTGCTAGGTTACGAGGGCTGGCTGGCCGGCTACCAGATGAATTT
TGAGACTGCAAAATCCCGAGTGACCCAGAGCAACTTTGCAGTTGGCTACAAGACTGATGAATTCCAGCTTCACACTAATGTGAATGACGGGACA
GAGTTTGGCGGCTCCATTTACCAGAAAGTGAACAAGAAGTTGGAGACCGCTGTCAATCTTGCCTGGACAGCAGGAAACAGTAACACGCGCTTCG
GAATAGCAGCCAAGTATCAGATTGACCCTGACGCCTGCTTCTCGGCTAAAGTGAACAACTCCAGCCTGATAGGTTTAGGATACACTCAGACTCT
AAAGCCAGGTATTAAACTGACACTGTCAGCTCTTCTGGATGGCAAGAACGTCAATGCTGGTGGCCACAAGCTTGGTCTAGGACTGGAATTTCAA
GCATAA

TABLE 38
MOUSE NOMENCLATURE
ICSGNM Nfatcl
Celera mCG18180
HUMAN NOMENCLATURE
HGNC NFATC1
Celera hCG15098
MOUSE SEQUENCE—CENOMIC
CAAACTCCAAGGAAGGTAAATTCTAAATCGAGTCTGCCAACGGAGAGAAGAAAGTGTGCAGAAGAGCCAAGTGGGACTTCCAGAGCTGAAAGGC
ACACTAATCCAGTAAGTGCTGGAAATGACGTCCCAGTGGGGGCTTAGCCATGGCGCTGAGCTGGCACAGGCAAGAGCCGGGGATGTGCTATTCC
AACTGAGACTGCAGGCTGTGGACTGAAAGAATGAGAAGAGATGTCGGGAGAGGAAGTAAGGGAGCGGAGAGCAAGAGCATATGCACAGAGGGAG
GGAGGGAGGGAGGGAGGCAGAGGGGTGCTGAAACCATTACAGATGTGTAACCCAATCTGCACCACCAGGAAAATCCCAGCAAACTCCAAGGAAG
GTAAACTTCAAGAGACCAGCAACAAACTGACCTCACAAGGAGATATATCAAAAGCAGAAGGTGGGGGTATTAACCCTTGCAAGGGAACCCCAGT
AACGTTAACATCTGACCCTAGAAGCAGCAACTGAGGCTTGAAGACAGTATGTCCAAAGGGCACTAAGAAAAAACTTGCAACAGACCCCTGTGCT
CAACAAAGCTTCTTTCTAACTCTTAGACGAATTAAAAGCTTTGGCACAGAAACAAAAGCCAAGAATACGCTGCTAGCACAAAGAACCTTCTTGC
GAGGAATACAAGGCAAAGGCGGGAATGCTGGGACAGTAACCTGCACCTACAGGAAGCAGCACGTCAGGAGGCAGGCACGTGATTTTACAAGGCA
ACATGCATGCATGTTTTCTTCTCATGACTCACTTAAAAAACAATCATATAAAACAATGTGTGTATACTGTAGTGCTTTGCCGATAGCTTACAGA
AACGCAACCCAGGTGTAAGATGTTTGCCAATAACAACAAATAGTGTCTTAAGGGACTGCAGAAGGCAAAGGAACAATTCCCACAATGCACCATG
AGGTTTGTCTGTATCGCTTTCTGTAATGCTGTAGAACATCACACCACTGGGCAATGGGAGAACCATATAGAAATAATGCTTCTGTGAGTCACTG
AAATGGTCGCAGGGTAAACTAAAACCCAGCATGAAGTCACGTGTAGCTGGCAAACACAGAGCAGACTCTAAGGGATGCTGCAAACCAGGGAAGT
ACCTTCTCACCAAACACATCCTGTAATGCAAAGAAAGCAATAAAGAGACTGAGTGAAAGGAACACGGAGAATGTAGAGAGAACACGCCAAGTTC
AACTTCATTACCTGAAACTGAAATGTGAACAGATTAACATTCCAGTCACGGGGCAGCAGTAGTCAAGCCGAACAGTCAAACATTGTACAACTTC
ATGCTGTCTTCTGGAGATAAGCTGTAGGTGGAGAGGTACACACTCACTACCAGGACGGGAATCATCCATCATGCACAAAGAAACTCCAGGAAAG
CCAGAGTGGCTGTGCTGCTGTTGGATGAAAGAAACCAAAAGTAAAAAATAAAAGTCAAAGACTGCTGAATTGGATGCTCAGCTCCCTCCAAGCC
TCGTGTATTGAAGACATGGTCCCTAGAGAGGGGTTGGAGCCATGGAGAGAGGGGGAAAACCTTTGGGCAGTGAGACTACCTGGTAGGAACCCCC
ATTTCTGGAGACAAGCCCTTCATGAGGTTGATCCCCACCCCAAACCTCCCTTCCTTTTGCTTCTCTGCTGAGATGAGGTTGTTGTGGGCTGTCC
CATGGTCCCACTGTGATAGGCTGTACTGCCATAGGTCCTAAAGCAATGGGGGCCACTCAATAATGGACTGAACAGGCCAAAAAAATGTAAATTA
AAAAAACCTTATTTCTTCATATGTTAATCATCTTTGTGTTTTGTTACAGTAGCCGAAAGCTAACTGATACAGATTAAGAGGGCTGCTTCAAGGT
AACTAAAGAACCAGTGTCTGAGAGACAGAGAGAGAGTTCTAAGTGTAGGTGTGCCTAACAACACAGTTCCCAAAGAGACATAGCAGAGCCAGCA
GGTACTTGGTGAAAGCCTTCAGAGTCAGGGCAGGTTGACTCTGTCAGGGGGTGAGCGTGACTCAGAGTGGCTTCAGTGACGAAGTCCCTTGTGG
CCACTTCAGGCAAAAGATGAGGAAATTGAAGAACACTAGGTATCAAGGGAAGCAATTTGGTCTTTATTCTGATTTCCAGGACATATGCCCTGGG
AGAAAGTTATTAATAACTGACTGATTAATTGATTGATTGTGGTATTCGTTTTATTTTGTTGTTTTAGGGCTGTGTCAATTTCTCATCATGAGAC
AAAATACCAGAGACAGCCACCTTAAAAGGATGAAAGCTTATGTTAAGTTATTGCTTCCACAGGCTCTTAGCTCCACTGCCTTTGGACCTATGGC
AGAGCAGAACGTTCTGGCAGCAGAGCACACTGTGGGCAAAGCTGGTGACCTTAGGCAACAGAGATGGAGTGTCTGGTCTCAGTATGCACTCCGA
GAGCAGTGCCCATCCCATGAGGTCCTGACTCTTCACACCCCAGCACAGGACAATGCTCCATCAGCCAGAAAACAGGTCGTTTGCCATGGCCTTC
AAGACTTGGGTTTTTTTTGGGGGGTCAGGGGTGGGGGCACCCAAACCTTTAGGAAATGGATGTTGCTCTGGTTAAATCAAATCATAGAAGGCAT
CTTCACTTGTCCAGGGACGAGGGAGAGATTAAACAGGAACCCCAATTCTGTGGTCCTCCAAGATACCCCAAGGTCTGAGAGGGAGTGGCTGATG
TTGCTOTAACTAACTCAACGGCAACTGTCAGGCTTCACTTGACTGTTTTTTTGTTTGTTTGTTGTTGTTGTTGTTTGTCTGTTTTGTTTTGTTT
TTGCTTTTGTTTGTTTGTTTTTAAGCACTTTAAAAGTAACCTGACTGGGCACCAATCAATCAGTAGGCAGCACATCAAGCCAGGGCTTTAGCAC
CAATGGCTAACCAATCAACTCAAACCAGCCAGGTGTGGTGCATGCCTGTCACCCTAGAACTGGGAATATTATAAGAGGACCTGGAGTTCAAGGC
CAGCCTGGGCTACCTGAGACCCTTTTTAAAAAATGATGCTCCTGAATTCTGGCCTGTTAATTCCACTCTCCCAAACCGGCTCTGGAGGAAGGCC
CAGTTTCCCAGCCTTCCCAGTGCCTCACAGTCCTGGGCCTCCTCCCTGTGTTGTGGAGTGAAGTAGATAGATCCAGTCAGAAGCCCCAGGTGAG
GTAACAGGAGGGAGTTGCAAAGTAACCATTGCTGTGGCCCACACTGCAGATGCAGTGAGGAGATGTGTATTTAGCTACTGATGTTTTACATGGT
TTACGTAAGCAAGCAAGATGTACATGGGTCTGGGGCTTGACTGTACAAAGAAGCCCTCCCTAGCCTGGTGCACCGTACCTGCCCACAGTGCAGT
GTGTACACAGCCAAAGGAGGCTCCAGGTTGCAGGACTGAGAGTCGTGCCCCTAGCAACACAGAACCCTGCTTTTGCACTAAAAGCCCATCAGAT
CTTGTTGGGGTTTTCTGTGCCCTTGTGTTTTCACACCAGGTTAGAATCAACAATTGCCTGGCTCTTGGAAACTTTGAGCGCTGGGTGGATAACC
AAATGTGCCCCAGAAGGAAGGATTTAGCACCCAAAGACACCTATTGGATTAAGCTTCTGGGGTGCATTTGCCCCAATAATCCACAGAAGCAACA
GTAATCTAAAAATAGTCTTGTTTTCTGTCCTAAGCTCCTTTTAACTTTGTTAGTCATCGCCAAACCTTAAAATAAAACCCGTGTAACGTCTCTC
CTGGTAGCAGCTATAAACAAACCTCCTCTAGAGGAGGCCGAAGGAAGAGGAAAGAGGCGGTAGGGACACGCAGGGAGCGCGGCCGCGGGACCAG
GCTTGAGCCCGAGGTGGCCGCAGGGCCTGGCGGACAGCTCAGAGCTGGCCGAGCTCCTGCGCCGCTGCCTCCGGAACTCCCTGCGCCTGGCGTG
CGGCCACCGTGGTCCCGGCAACGGCATTAAACAGAGGGAAACAGGCCCGGGATTCCGTCACCCSCGGGGCATAAGGAGGGCATTGAGACAGACA
GGAAAGGGCTTTTCTGGATGGGGTGAAAGTTATTTATTGGAGAGGGAGGGGGGAGNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNCCTCGCCGCTGATTCCTCCGCCCAGCCCCGCCCCGCCCCGCCTGGTCCC
TGACAGGGGAAGGGCGGGGACGGAAACTCTTCAGGCAAAATCTTGGCGTGCTGGTCTGGCGACACTAGGACTCACTGAAGAATGAATTTGGGGG
AGGGGGGTTGGAGGATGGTTCTTGGGTAATGGGGTCTTGGCACCCCCTAGCGGCAACCTCTTTCAAGGAGAGCAGGGAGGCTTCCGCGCATCCC
TGCAGGTTTTGAAGGTACCACTTTGGCAGCCTTGCCCGACATCCCAGCATCTTCACTGAGCATTCAGCTACTTAATAGATCATGCTCCACACAC
CGGTGTAGGCACCTCCAGCCTAGCTATACTCTAGACCACCTGCGTGAGTGGCTCCATCCTCTCAAAGCCAGAGGTAGAGAGGTGCGCGCCATGC
CTAGCTCTCCATGTGAGTAGCAGGCCAGTAGGTAGTGCGCCTGAGACATGTTGGGCACTGCCTGTGGCTATGCAGCATCCACCACTGCGCTTCT
CTGCCGTGGTAGAAAACAGGATGGCTTTTAGTTCTGTGGCGTTGAGGCAACTAGAAAGAGCCGCTAGATGCTGCTGGAAGAGTGGATGGGTTTT
AGCCAAGTTTTCATATTGTTTAACATCGAGCCCAGTTGGCCAGCTCCATTGCTGGTTGGATCAGATTCTCTAAGTCTTGGGGTGTGAAGTACTT
TGGAGCACCTACTGGCTCACTACACGCCCCGCGCGCCCCACAGTCTCGCCGCCCCACAGCAGACACGCTTTTTTTTTTTGCTTTTGAGTTTATT
TTAAAGCATATGAGGTGTAGGCCAATAGATTTATGATTTCTATACACCAGACGTTCTAACTGCCCAACCCTCCAATCCGTTCCGTCCAAAGCCA
ACAAGAAAATATGGTCGGGTTTTGGCAGCAGAGTTGCACCCCTAGGAGATGCGGGGAGATTTGAAAGAGAAAAAGTGTGAAGCGAGGCGGGACA
GAATCCTCAGAAGTTCCCCCAGCACCAGCCTCCTCCTATGCTAGAAACCTCACGTACGCGCCTACAAAGCACCCTATGGTCCCTGCACTGGTCG
TCCGCACCCGGGGCAAAGACCCAGAGGAGGAGCTGCGCAGGTGGGCGCGNNNNNNNNNNNNNNNNNNNNCTGGCCCTTCGTGCCCGCTCCCCGC
GCCCCGCACCCATCTCGAGTCCTCCTCCCTGGCTAAGCTGCCAGGGCCGGTCTGCGCGTCCAGGGAGGGGCGCGGGCGTCCGGCCGGACAGCGT
GGCACACACTGTGAAGGGTGTCCCCCTGGTGTTTCCCCGGCGCAGCCCAGATTTCTAGCGCGCTTTCTTGCCAATAGGTGTCTCCTCTTCTCTA
GCGCTGAGTACTCAAACTTGGTGGCGTCTTGGAGATACCACATGGGGAAACTTGATTCACGGGGGCGTTGAGATGTGAGGATGGGGCGCAGTGG
GGGCGGGAGCGCTAGAAGCGAAAACAAACAACCAAACAAAAACACCCTGGGATGAAGTGAGGCATCTTTGGAGAGCCGAGTTGGTCCTGCCTTT
ACCTCAGGAAGGCAAGCAAGTCCATGACCAGAAGTATAAGAGTCCTGCATCCTCCCAAGTGAGTCTAGCCTTAGCTGCTTCTCACTAAAAGTAA
GAGCTTGGCAAAGTGGCCCCAAGTGGTCCAGACTTCTAAATGCCTCTCAAACTCGTGGCCAAATGTTGTATCCAGACAAGAGTTCTGCCTTAGA
AAATGCCTTGAGAAAGATCGAAGACGGACTGCCAGGTTTGAGGTTGCTTCTTCCAAAAGAATATGCGCTGCAATCTGCCCTAACCTGTCAGGGT
CACAACAATGACATGACCCAGACCTTCTGTCAGAGCCCTGCAGGTCCCCTCAAAAGAGTTAGCAAAATAACTTCTAAAACGTTTAGGCTCCTGC
AGGTGTCTTTGAAACACCCTTTAAAGAGTGACTAGCTTTTCAGCAGGGATAACTTGACCTATGTCGAATGGACCCCAGGAGCCATAGGGCTCGT
AATTCCTTTTTGGCACCAAAGAAGTTACTTAAAATTACTGAAGGCCAGCTGGACCCCTGCTGGGCTGGGGCACTCCTATTTACAAGGGCGGCCA
TCCTCTAGACAAGCACTTGGAAGGAAACCTTAGCTTCCTGTGAGAGTCTGATGAGTGGGTCAGCAGGCAGAGGCAGCCGTTCCCAGAGTCAGCC
CTGGACACCTGGCTCATCTTTAGACTTGAGCCATCGGCTCAGCACAAATGGATCCAGCTTTTCAGAGCCCAGGTGTCCTGGGTAGAGAGTCTTG
TGTGAACTTTGAAGCGGAGCAGCAGAGATTGTTCCTTTCATGTCGCTGACCTCAATTCTAAAAGGTCTAAATGTTGCTAAGTGGACTCCCGAAT
GAGCTCTTGCAGCAAGGAAAGCTACAGTGTTTTTGTTGTGGTCTGGGTCGTGAATTTCTTGACATTTATTTCCACACACATCTGTCAGTCACTT
TTCTTCTCCATCTTCTTCTACTTGGAGGCCTTAAAGCTTACGTTCTTTACTTGTAGCGTTACTGTTCCATTACTCAAGAAACCTTCTAAGCTGA
GGTTGATGGCTCACCCTCGTAATCCTAGGCGTAATCTCAGCACCTGAGAAACTAAGGCAGGAGGATTGCCATGAGTTTGAATCCAGCCTGGGCA
ACAAAGCAAGAACCAGGCCTGCCTTGGGCTACAATGTAAGACCCCAAGTCCCAAAACAAAACTTTTTTGTCTTTCCTTTTTTTCTTTCTTCCTT
CCTTCCTTTCTTTTCTTTCTTCCTTCCTTCCTTTCTTTTCTTTCTTTCTTTTCTTTTCTTTCTTCCTTCCCTCCTTCCTTCCTTTCTTTCTTCT
TTTCTTTTCCTTTTCTTCTCTTTTCTTTCTTTCTTTTCTTTTCTTTTCTTTTCTTGTTTTTTTGTTTTTTTGTTTGTTTGTTTTTTTGTACAAA
AGGGAAAACTATTGGGCAGAGGGTGTAATCATTTCTGGTGTAACTTACCTAGAAAAGGAAGTTTGACAGTGTCCCCAATGAAGCAGTGAGCTGG
AGGCAATAATGACCGAATGATACTGGTCATATTTTACAGAGTGTGTGTGGATGTAACTGAGCTAACTAGAAACTGTGACCCAGTCTTGAGACAG
CCAGACCCAGGCAGGGGAAAAGAACTGTATGCCGCAGGTCAGTATCTGCCATTGCTGTGGGCCGTGGGAAAGCACCTTGAGGAGGCCTTGATCG
GGACACCAGCCTTCTCTGAGGCCTTCTTCCAGCTTGATGGGCATTTGGATGGAAGCTCATGCGGCCTGGCCATTTTTGAAGGCCTTGTGCTGCT
CAGCTCTGAAGAACTCAGAGAAACACAGGCCTGTAGGTAGTCACAGCGTCAGAGTTGCCTCACACAATGCGCCACAGGCTCTCCGGAGCTGAAC
AAAGCTTTCCCTGTTCTCCAGCTGTATAGCTTCATGGCACTGTTTCTTAATCCAGATTAGAAAATGCTGCAGCCATGCACCCTGCTTCATCACA
CCCAGCACCCACCCGGGCTGGCCCATTAACATCTTGCACAGTTAGGAGGAAATGGTCCAAGCTAAACACTTCTATCAAAACTGGAGTGTCTGCT
GAACTGGCAGACCAAGCTTCATACTATATAGAGGACTGGCAAGGGGGCTGGCAGAGGCACAGGGTAGCATTTCATAAGTAGTTGTGTTGACTGA
CTCCTGTGTTAGAGAGTAGACAAGGAGGGAGGCAGGCCAGCCTCTGAGAGAAAGGCCTTGTGACTTCTTTCCTATTGTCCCAACATCTGGGGGC
ATGTGGCCAGGGGAAGCCAGGTGCTTTGTAACTTTAGCATAAAGCTTTCTTCAAAAAGTAGACAAGAAGGGCTGTTGTGTGTGGAACTGTGGGA
AAGGTGGATAAAGAGAGATCGAGGTCTCACGGCAGAAAACAGAACTTCCATTTCCCCGAAGGTCAGCAAAAAAGAAAAGTAAAACAAAACAACG
CTCCAACTCGCATCTCAAAGTGAGAACTATTATTAGAAACGAAATCAGGCCAGACTGAACTATAGTTTATCACTGGAGAAAAGCAGCCATTGAA
ACAGGGTCCTGGGAGAAGTGGTAGCCCACGTGATGCTCAAGTTAGCCTCCGGAAAAAGCCATCCCCCAGAGTCCAGAGCTGCAGGGCAGGAGGA
CACTGGGGACACTTGCATCTGGTGGCAGATACTGACACTAATCTCCCTCCTCCTTTCCTTCCTGCCTCCTTCCAGGGTTGGGTTCTGATACTGG
TCTCCCCTGCCCTGGCCACCATTTTCCTGATATTCCAGATGTTGGTTTATGTGGTGCCAAGACACCAGGAAAGTTAGAACAAAAAGCTGGGAGA
AACAGGAAAAGATGGTTGGGGCTCTGCAGGCCCAGCCTTTTCCTTCTGATTGCTGTGTGCATGGTACCCTGTCTGAATGGGTCACACCCAGCAC
CTACTCAGCCTTTGGGAGCTTTGAAATGACCCCCTACTGAAGCAGGAGTGTGTGGTGGTTTTTTTTCGGAGCTGAAAGATTAATGAGGTCGTTA
GTGCACGAAAACAAGATTTGCTTTGAGGTAGGTGCCCACAGAGAGAGGTTGGCCACCATGGGCAGACTTGAATGCTTCGGAAGCAACTGTGCTC
TGGGGTTTGACCTGCATAGAGGAGAAGGCGACCCTCAGGAAACTCCACCCTCATTCTCCTGACCCTTTCAGGTAATTTTAGTAAAAGCAGGGCT
AGAAAATGACCCCACCCCCCAGCGTTGATTGCTTCAAGTTGCAGCGCTCTGAGGTGGGCTCTGAGATGCTTCCCTGAAAGTGGGTTTCCTCGGG
CAGCAACCTGGAGGAAGCCTAAGAGAAGCCATGTTGGGAATATACATATATAGATATATCAGGGTTAGTATATATTTCCCTCTTTTCTTTTCTT
TTCTTTTCTTTTCTTTTCTTTTCTTTTCTTTTCTTTTCTTTTCGAACTTTTGAACTAATTCAGTAGTTTTGGTCTTTTTCCTGGTGCAGGGAAA
CAGCAAGACCCAGGGCACCTCCTTCGGCTGTGGACTGCACACCACGCTGAACAGGAACCAGGGGACCGGCTGCGGGGCCGGGCGGCGGGTCCAG
GACGCAGCTCCAGCCCCGGTCAGCCCTGTACTGGGTCCGGGAGCGACGGCTCGGCCCGGCATAGGCTGCTGCGCTCCCCTGGAGGCGGTCAGCT
GCGCAGAGGTTGGGGCGCGGACGCCAGGGCTGCTGGCAGGGGGGCTAGGTTCCTGCGGAGCGGGGGGTATCCTAAGGGAGCGAGCCCCACACAG
GAGGCGGAGGCCCGAGGGAGCTGTGCGGGGTCGTGCGGGGGAGCGGGCATGCGACAGAGGGCCGGGGGTGGACCTCGGACCGCGCGGTCCTGCG
GGCACGAGTGGAGCCTGGGCTACGCTTGGGCTCGCGCGCGGAGTTTATTTAAAACTAGGAAGCCGGCGGCCGCGAGCCGCTTGTTTATGTAAAC
CCGGAAACACCCGGGGGGACAGGGGCGGGACCGGGGCGGGAGGCCGGGGAGGGGACAGGGGAGGGGACAGAAGGCGGGGGCCGCGTCACGCCGG
GGACGCGCGCGAGCGCCGGGCGCACCAGTGCCGCGCGACCCCGACATGACGGGGCTCGAGCAGGACCCGGAGTTCGACTTCGATTTCCTCTTCG
AGTTCGATCAGAGCGGCGGGGGCGCCGCGGNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCA
GCTCCCTCAGCAGGAAGGCTCCACCACTTGGGTTTCCAGTCCACACCTCAGCTACCAGAACCTGAGAGCTTTCGCTATCTTGCTGTCTTTCATT
TTCTCTTCTCTGTCAAAAGTAAAGCTTCCAGTTAGGTTAACAATCAATGTGCTGTTTCCCCAAACTTGAATCTCCTAAACGGTTGGCAGCCCAT
TCCTTGCACCAGTTTCCCTCCTGGGCAGTCTGGCTCTTGTCTCCACCTGACTTTCTGTTCCAAGTTGCACACAGTCCCGGACAGTGTTCTGTGC
TTTCCTTGGGGTCTGTCTCCCAGCCGCACTGGGACAGAACAGACATGACACTTGTAGAGCATCTCCGATGATCTGATCGTGTCATGTATTCCAC
TGACGCTCACTACTATTTTTTTTTTAAGTTAAACATCCTGTTCGCTTTTGAAGGTGAATGCTCCAACTTGGCAGTCACCACTCAGCAACTGTCA
TATGTCCGCCCATAAGACCCCTTTCGGATAGGTTTTCCTTCTTCAGGGCACAGGTGTCAGGGCTCCGGGGTCAGTGACATGAGGGCAGGCAAGA
CTGCAAGAGAGACAACCATTGGGAAGAGATCTGGGGCTAGAAGTGGAGGGAGACCCTTGACATCTCTTGGTCCTGCCGCTTTGGGGGATTTGGA
TTTACCCGCTCTTAGTGCATGGGGGCGTTTTTGAGTTCTCTCCAGAACCCTCACACTCCAGGCAGAAGAGCTGACTCTTGACTTCCCCTTAGGG
TGTCCATGTTGGATTTGGATGAGTGTGTACCACTGTGCTTGTGGTTCCCTTAGGATCCGTTAGCTCTTGTCTCTTTAAGGGGGAGGGGCTACAG
CCATGGAGCCTTCCTGGGCTGTCCAAGCTACCTGCTGTTGGGATAGGGCCCCACGGGCTTCCTGTGACTCCTTACTTACACTGAATGGACAACT
GACTACAGTCCCAACACTAGGAACAGACACAGAGATCCAAAACGGGTACTTTGGTACCTCATGGTGGCCTGAGGACTGCTTGGGGAGAGGAGAG
GGTATCAGGGATGCTGCAGACTAGGTAGTCATCTCCACAGACTCAGAACCTTCGAGGGAAGGGTGCTGCACTTGTTCAGGTATACACCATTCAT
GGATCTCAGAATCCAGAGTTCCACTGGAGTCTTCGACTCATAGCTGGACACAGCCGCTCTCACCGTGTCTTTCTGAACTGTATTGGGACACATA
ATGCCCAAGTTTGAGGTTCATCTGGGTCCATTCATGGAGGGCACAAAAGGAAGTCAGTTTGGGTGCCAAAGGAGACACTGGTAGGGGCTCTGGT
GAAGCCTGGGAAGCAGGTGGCTCCTTGAGCTTTGGATGACGTCCTCCCCTTACGCATGTGAGGGGCCGTGCTCCTCAGACATGCTTGTGTTGGT
TATGCTCTGAAGTAGATGAGGTAGAAAGGTGTCTTGTGTCTCACTGCAGCCCAGGCCATTTGTGATGATACCAGAGTGCGTCGCTGAACTCCCT
ATTGCTGCACATAGGTGAGTGCTCCCTGGAGCTCCGAGTGCTCCCTGAACTCCCTATGGCTGCACATAGGTGAATGTTCTCTAGAACTGCACAG
AGCTGTAGGGACATGCCTCTTGGATGGATGTCCCTGTGAACTGTGGGTCACTGCCTCAGCAAAGCCAAGATGCTACATCTAAGTCCAACACCTT
TAGCTTGCAGGGTGTAGGCCACTGGAGAGAGCCCTTGGCAAGGGGACCAGCCTTGGGAAGAACTTTTTCTCCCTTCTCTTGTGTTGACAGAAGG
TGCTAGAGAACATTGGACATGTACAATACATGTTACACTTCTCAGTTTAGAGACAGACAGACAGACTGACTGACTGACTGACTCTGACTTTTTT
CTCTCTTATTACCAATCCCGTTATTACATTTTTATCCCTTGTCCAATTGCATTCCTTATATTACACAGTCATCTGAAGTTACTAAGCTCTCTGC
TCCCCACTGAGCAGTAGAAGGAGCTTCACATTATCTGGAACGAGTCTAAACATAAATTCATGTAGGCCTGACCGATTCTGAGATCAGTTGCAAA
GGAATATTTGGCTTTTGCAGCTTGTCCTGAGCACACAAAACCCACATCATGAAAGCGGACATGGCTACCTCAGAGCACAGCTTACCTCCTCAAT
GCTGCATGAGGGACCCCAGTATCCCCCTCCCCACCACACCACACCACTGTCTCCCAGCTGGGTTTCATCCATAGGCAGAGACAGACACTCCACA
GGCAACCCCAACTACCAACGACTCTTCACAACTTTCTTCAGATGAAAGCATGTGAGCAGAAATATGACTGCTTCATCTGTGGTCACTGGGGCAT
GTGGCCCTGGACACTTCCTCCCAGCCAGGGCTCTTAGCAAGATGGCAAGTCTTAAACTTCAGACTGGTGAAGGCTGCAGAGGCAGAACACTTCC
AGAACTGACTGAGAACTACGAGAGCAGACACTGTCTACTCGCCCTCCTGGGGACACAAAGTCACATTTTGGTTATTCTGAGACTGGCAACAATT
AAAGTCTAGATGGTTACACTTAAAAATCCTGAGCTCAAAGCCTCAAAGACTGATGTCATCTTGCTCTCCCGAGAAGGGGGAAGTGCACTAGTTC
CGCTTTCTTTACAGAGTANNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNGAGCAGCTTCACTGGGCTCAGGTTCTATAGATGTTTTAATCAAGGACAAACCTGTCTGTGTGAGCAGGATAT
GCTAGCCCCGTATCAGGTTTTCAAAAGAAACCCTTGGCTAAGAGGGAGCGCCTTGGAAATACAACAAGGGAAAGGAATGCTTTAATATTCACCT
GGGGGAGTGTCGGAGGTCTTCGAGGAAAGATGTGGGCCCAGGCTGGACACGGGACCCTTAAGCACGCTGTAGTGAGCTTATTTGCGGACCATTG
GGTGACATTTGCTATTGCTCCAGCAAGTCCAGCTCTCTGCCCTGTAAATCTCCCTTTAGCACATCGTTGCATGACAGTATTGAGAACCTGTTGC
TGTCTGTCACTTCTCGGGACTGACCCAGTCTCCATACAGTCCTCATTAGGGAGCACTATCCAATGTGCTAAGTAAACACAAGAACGGTGCTGCC
TGCCCCAGAACTGTTTTGTATTCTTTTGTTTCATCCCTAAGTGTGGAGACTTGCAGCCTCCCTCACACCCTGCAGGCTCTATTGCATGTAGAAA
GAGCAGGGTGGGCAGGCATTGGAGGCCAGGACCTGGCTGGCAGGATCTTTCTGGTTCTATTTCTAGCGCAGCCCCAGGAAAGGCTAAAGGGGTT
TGTTCCTTAAAATGAAAGGTGAAAGTTGGCTGTGATCGGTTGTGCTCTGTGCTTGTGAACAGTGAGTTTGGGTGGTGACTCCGAAGGCCATACC
GATGGCAGAGGGTCACATATATGTGAGCATCACATACACGTGGTGTGGCAGAGACACAACATTGTCTTCACTCCAGATAAAATACCAAGTGCCT
TAAGCTGAAGCACCTACAGGGGTATTTCCCAGCGGGTGCCACAGATTGCCCAGTGGGCAGCAGGCTTTCTCCACAAGTCTTAAGAAGACATAGA
CCAGGGGCTGTCATGGTGTGCAATGCCCTGGTACAGCTGCAGTAGTACCAGCTTGTTGAATTTGCTGAGGCTGGTGGGAGCTGGCCAGCGCAGA
GAGTTCTGGATTTGCTCAGCATCGCCTGCCTGCATCTCATCCATGTGAAATACTGAATGCCGCAGGGTGATTGAGAGAAATGAGCTCAGAGGAA
TAGGAGGACAGACGGATGGACGGCGACACCTGCACAGGGTGACCGCGCCTTCCTTTTGTATTACATGGCATAAGAGAATGTGTGCGTGAGAGGT
CTTTGCTTCCAATTGTGCTTTTCTTCCCTGGTCTCCCACACCTCAGTTTACTGGCCCAGTCAGCAAAAGCCAGGCTGCGGGGCACGGATGTTGC
CCCGAAACAGCAGCATATGATGTTCTCTAATCAGCCTCAATGTCTCAGGATTAGTAAGGAATTACTGGGAAGCCTGGCATTCCTGATAGACCCC
CATAGAGTGATCCCTTTGAGGAAGTGGAAGTTGGTACAGGTGGAGGGCTACCCCACAGGTCCAGGCTTTGCAGCACCAGGGAACTCAACCGGAC
TCAGCAGCCTGCCATGGGATCAGTGGGCACTCATGCTCTGAGGACTCTGAGGAGAAGGCAGCCAGCTCTGATGGCTGTCACAAAGCGAGCGGCT
CTAGGCATGTAAGCTTTTGTTTTGGAGTTTGACCAAGGAAATCCATGTGTAAGCTAATTGCTTTTGTTCTTAATAGGTTATTTTTTGAGAAAGG
GTCGCACCTAGCCCCAGGCTGGTCTCTGACTTCACCATGTAGCAGGATAGCATCTAATACCTGATCCTCTTGCCTGCACTTCCCGAGCGCTGGG
CTGATGGGTGTAGGCGTGCTTATGTGGACATAGTCAGTGTCGGCATTGAACCCAAGCTCCCTGAAGGCCGGACGAGAGCTTACTAACTGAGCTC
TACCCCAAGCTCCAGTTCTGTTTTCTTAATAAACCGTTTATGGATTTTCCTTTGCCCCGTTGAATCATATTGTATTCAAACATTTTTGGTTAAA
GTAAAGGCCACTCTTGTAATGTCCTGGGGCCCACGTGGTCTTTTCCACAACTTCCTCTTCCTCTGCATCTGGCCGAGCCCTGAGTGAGAAACCT
CCTTCTTCCTCTTTCTCTGGGCTTCAGACACCCTGTTGCTAAGGCAGCCCCAAGGAGACAAGCAGAGATTTGCCATGTCGTCTGCCTGCCACTC
AGCCATTTTTCCCCAGTACCCCAGGCAGCTGACCCCTTGGCCTAAACACTGCAGGGGTGTCAAGGCAGGACTCAGCTTGGCTTGCTGCTAGGAC
TTTCTAGAAAACTCGAAAACTCTTCCAATTTTCTCTTCCTTCCAAAAGAAGGTGGTCTGCTGTGGGGCCAGGACTGGTCTGGATTAAGGCGGAT
TCTTGCACTTAGGGGACAGAGAATAGCCAGGCCCTGCCTAACTGCACGTGTGCCTCCTTTGGGATTTGGCAGCTCAAGGCCATGCAGGACTGGT
TTGGGTCTTCAGCATCTGGAGACATGGGCAGGTGCTGGGCTCAACTTTCCCAGTGTGTGCACAGCTGCTGGGATACCCATGTTTCCCATTAGCC
TTAGCCTCAAACTTCCTTTTCTTCTGGGGTTGGGTTCCCTTAAGATGAAAACATAAAAAAAAAAAAAGATGTATTTATTTTATGTGTGAGCATT
TTGCCTACTTGTGTAGATATACACCACATGTGTGCTTTGGTGCCTACCAAAGTCAGAGAAGGACATCTGATCACTCTGAAACTATAGAGTTACA
AATGGCTGTAAACCATCATCATGTGAGTGCTGAGAACTGAACCCGGGTCTTCTTGCAAGAGCGAGGAGTGATCCTAACCGCTAAGCCATCTCTC
CAAACTTGAAAGCCCGTTCTTTTCCAGTGTTGTGTTGTTGGCAGTCTTCAGACAGTGCACGGAGCTCTAGAGACATAGGGCAGCATATAACATT
GGCAAAACCTGACTTCCCAGATGGCTTAGCTGCCTGCTCAGGGGTAGGGCCTCTGAGGCCTGGGTGCTGTCTCACATGGCCTGGTTACCACCAC
TCTCCAGATGGGTTCCTGAAGCACCTTTGTTTAGAAGTGGTGCCTCTGGTAGAAGTTCGAATTGCTGTGTGATGTCCCATATCTGCTGGGGACT
GTGCCCTGGGCTCTAGTATGGAACCCTTCTAGGACCACAGTGGTTCTGAGGCCCAGAAGGCTGGAGAGAGCATAGATGATATCATCCTGAGGGG
GCCTTTTAGGTGATGAACAGGTGGGGGTCATGAAGGACACGCCCATCTGGGGGTGGCAGTGAGTGTGTGAATTTCTGAAGACCTTGGGAGATGC
TCAGAAGGGGCCTGGTTGGATGTGAGTGCAGCGAGAACTACTTCCTGACCGAGGCATGCTGCGGAGGGTGTAGTATCTCAGGCAGCACCAAGCC
AGAAATCCAAGTGCAGAGGTGAGGCTGAGCCATGGCACAGGCATTCTTCCCGGACTGTGAGATTCCAGCATCAATGAAATCAATTCCTAAAACT
CCTACTCTATTAGCCTGTAATTTCTTCCTCCTTTTTATCATTAGCAATAATGCAGTGACATTTTGGGGTGCCCACTTTCCACTCCTCACTGCCT
TTGATTGACTAGTTAAGACCTGACTTCTTACCCGAAGATGAGCAGATCCCGGCCATATCTTCTGGGCCATGGAACATTGAATGTGCCTTTCCAT
AGAAAGTTTTAGATTGGGAAGTGCTAGGCCAGCTAAGGGAGATCTCTCCATCAGTATCTTTCTCTGTGTTTGTCACCTGTATGGGATTAAAAAA
ACATAGGATGCATAGAAAACCCTCTTCTCTCCTTTTCTTTTCTCCTCTGTCTCTTCTTCTTCCTCCACCTCCTCCTCTCCCTCTTCCTCCTCTT
CCTTCCTTCCTTCCTTCCTTCCTTCCTTCCTTCCTTCCTGCCCCACCTCCTTTTGCCAAAATGAATGTGTAGACGGCCTTTAGTGAGAACAGCA
GGGCAGCACAGCTCTGGACATAGCCATCTGTTGTGCTACTCCTGAATAGGGTCCCTGGTAACCTGTTCCAGACTTTTCTGTTGGTAACTTCACA
GAGGAGCCAGACCGTCTGTTGGTCTCACCAACCAGCCTTTGGTCATCAGGCTTTTCCACCTGAACCTGTTTCGTATCTTCACCAACTCGGTTCT
TTAGGATTGGTGTTCTTTTCTCACCATGGACAGTCTGCATCTCCCATCAGTCCTCCCTGGGGCTGCATTTTGGGATACCAGGGGTGGCCTGTGT
CTGCTGGACCTGAAGAGGGGGGGTCACTGGCAGTGGACGGCTCTGCCCCTTGTCCAGTGCTTCTAGAAACAAACACTCTGAGCCTAGGGACTCG
GCAGAAGTGTCTGATAAGTCCCAGATGCCGCCAGCCACAAGTCCTGTCTCTCCAGAAGTATTGCTTTCTCTAGAAAGTGGCTTTAGAGTTATTT
AGGACAGGTGGAGGTCATTTCAGTCTTCGGCTCCGGCCTCTTGAGATCTGGATTGTGGACTGGGGGGTGGTGCTTCCCTATGACAAGGCTAACC
ACAGCCACGGCTGTAGGCTCCACTTGGCAGATGCAGGTTCTTGCTACGGGTTGCTTCCGATGCCTCGAGCTGTACCCCATTCTCCTCTTTGTCT
CAGCTCTGGCCTTTCCAGCGCGTCTCTGTGACCCAGGGATTGAGGACTACCGATGGCCTCTTACTGGACACTGGAGTGGGACTCTGGTCCATCT
CATAAAGAGGCGGCTGTGTAGCCAGAGAGGGTTTGTGAGCCCATTAATGGTAGAAGCCTGGGAGAGAGCAATAGTGGGAACTTCCTAACCTACC
TAAGGACTGGCATGCTCCTTACAAACCATCTTAGGGTTTGACCGCTGCTTCGAGACCCTTTTTCTGGAGTTTCCCCCTGTTGGTTTCTGAGCCA
GCGCTGTCCTTGACCACAGTATCACGCAGCTGGGCGCTGAACCCTAGTGTGGATCTAGGACTTCCTTGGGCTTATTTCCATTATTCAGAGACCT
GGAAATGCTTGTCTCCAGACTCGTGTAAATGGGCTCTGCGATGTGGAGAGCCTTTCTGATCAGAAATCCGGAAGCATGGCTTTGCTGTCGAGTA
CTAATTATAACAATGATTTTTTAAAAAGTGCTTACCAGAGTATAATCAGCACACAATGATCTCGAGGTCTAGGGGTGTCTTAGGAGCCTGTGTT
TTGTTTTAGACGCTCTAGAAGATAGATTCCCAGGCACCAGCTAGCCTCAGGTAGCCATAAGAGCCACACTGGAATCCACATTGTCACCCTTCCT
CTGGGTGTGTTCTTTGAAGCCAAACATTTGGCCTGCTTGATAGAGTCATTTGCTTGTTAACACCTATTTAAACACCTGGCTCCCTGCGTCTGAA
GCACCTCCCTCCACCGATAACACCGCCACTCTTCCTCCCTCCCTTCACAGTCTGTCTGTCCATCTTTCCTTCTGCCACTGCCCATCCATCACCT
GCCATTCAGGTGATGGTCTGCCTAGTCATGAAACAAGATAAACGTGTGATGTAGAATCTGAAATACCCCTGCCTCTCTCAGCCTTTGATATACT
AGAATTGCAGAGGTACTGAGCTGTGTTGTGCCTGGCACCGTCTGAGGCTCTGCAGACAGCCGTGAACCACAGGGCAGGCTGTGAAAGCTGGCTT
CTGTTGGTCAGAGAGATGGATAGATAAAAATTCCTGTTAAATTGTGATATATACCAGGGTGGTGGTGGGGGAGGAGGTAACTCAGCTATTGGGT
AAGGATTTGAAATCACAACTGGCTAGGGAATGTGCATTTTGAATATAGAGAAGACAGTAAGAGCCAGCCATGTGATGTGAGGTGGAGGACATCT
TGCCTGAGGAATGCTGTTGTGCTGGTATAGAGTAAGGTCTATAGTCTAGCAGGAGGCCTTGCAGCAGTCTGACCTCAGGCCATTCCCAGAGGCT
ACACTCTGGGCCTGGAGTCCTCAGGGCTGGCCCCAGGTCTTGGACTCACTGCCTTTGTGATTTATGTGTGTCCTGCTCACTGGCCTCACTCTGT
CTTTCCTCTTTTCTTCCAGAACACTACAGTTATGTGTCCCCTAGTGTCACCTCGACCCTGCCCCTTCCCACAGCACACTCTGCCTTGCCAGCAG
CATGCCACGACCTCCAGACGTCCACCCCGGGTATCTCAGCTGTTCCTTCAGCCAATCATCCCCCCAGTTACGGAGGGGCTGTGGACAGCGGGCC
TTCGGGATACTTCCTGTCCTCTGGCAACACCAGACCCAACGGGGCCCCGACTCTGGAGAGTCCGAGAATCGAGATCACCTCCTACCTGGGCCTA
CACCATGGCAGCGGCCAGTTTTTCCACGACGTGGAGGTGGAAGACGTACTTCCTAGCTGCAAGCGCTCACCGTCTACAGCAACCCTGCACCTGC
CCAGCCTGGAAGCCTACAGAGACCCCTCCTGCCTGAGCCCAGCCAGCAGTCTCTCCTCCAGAAGCTGTAACTCTGAGGCCTCCTCCTACGAGTC
CAACTACTCCTACCCATTACGCGTCCCCCCAGACCTCTCCGTGGCAGTCACCCTGCGTCTCTCCCAAGACCACGGACCCGGAGGAGGGTTTTCC
CCGAAGCCTGGGTGCCTGCCACCTGCTAGGATCGCCCAGGCACTCCCCATCCACCTCTCCTCGGGCAAGCATCACGGAGGAGAGCTGGCTCGGT
GCCCGCGGCTCCCGGCCCACGTCCCCCTGCAACAAGCGCAAGTACAGTCTCAATGGCCGGCAGCCCTCCTGCTCACCCCACCACTCACCCACAC
CATCCCCCCATGGCTCCCCTCGGGTCAGTGTGACCGAAGATACCTGGCTCGGTAACACCACCCAGTATACCAGCTCTGCCATTGTGCCAGCCAT
CAATGCCCTGACCACCGATAGCACTCTGGACCTGGGTGATGGGGTCCCTATCAAGTCTCGAAAGACAGCACTGGAGCATGCGCCCTCTGTGGCC
CTCAAAGTAGAGCCAGCTGGGGAAGACCTGGGCACCACTCCACCCACTTCTGACTTCCCACCCGAGCAGTACACCTTCCAGCACCTTCGGAAGG
GTGCCTTTTGCGAGCAGTATCTGTCGGTGCCACAGGCCTCGTATCAGTGGGCGAAGCCCAAGTCTCTTTCCCCGACATCATATATGAGGTAAGC
AGCCTCCGACTGGAGTTGGGCATACGGTAGGGGTGTGGCTGGGGTTGAGAAGAGCCTACCCTGGCTTCTCTCTGGAGATAATGGTATGCTACCA
TTCATAGCATTATGACAGTCCCCTCTGTCCCTTCTTCTGGTGATAGCTGTCTCCGCCTGCTTGTTCCTCTTCTAAGGACACCTTGGACTAGACT
AGGGGACCCTTACTTTTTGGAGGCGTGTTCACAGGTGTCAAGGGTTGCAGCACATGAGTTCCGTGCTGCTCTACAGCTCTGNNNNNNNNNNNNN
NNNNNNNTTACAGGGAGGTCCCCGCACACATGCACAGCTGTGTTGGTAATAAGCCGAGGGAGGCAGCTATCCTCCTGTGGATGTTCTGCCTGCT
GCTCGCCCACTTCCTCTCTCTATGCCTTAATCGCTATCTATCACCATGCTGGGGCAGATGGGCCCCTCAGCTCACAGTCCCTCAGAGCAGCCAG
CTCACCAGCGTGCTACCAGCAGCACGACCACCACCTCCCGTCTTGCTGGCCGTCTGCTATCAGGGAGTGTGTTACAAACCTCTGGTCTTAAGCC
GTTGGTTTTTCTTTTTAACGGGATGCAGGGGCTGCTTGCGATTTACCAGGTCCATCGAACCAGCTCAGTGCTTTTATTTTTAACTAAACAGCTT
TCCTGTCAGCTGTACCGCTTGCGACGCCTGGAAGGCTGCTTTCTGCTCCAGGGTTTTCACTTGAGGCATGACAACATCCACTCAGGCCCGTTGA
ATGTCCGTGTGAAATCAGTAGGGATGTTTCCATGAGCTGAGGCACCTGTGATACCCAGGGACTATCATTTGGCAGGGACAGTCTAAGGCCTGCT
GCTTCGCTTTTGCCTCATTCCTCCCCCCCCCTTCTCCTCAGGGCAGACTCACCCCCCCCACACACACTCTTGGGGCATCACCACCTGGTATTTG
GCCCCCTGACCTCTAGATGTAGACAGTGACTATGAAGCTGTTAGCTCCATGCAGTGCAGCCACGTGGCCGTGTCTTGACGGGTCACTCTGGGAT
GTGGGGTGGCCAGCTGGGAGCGGCACTGCTGAACCTGTCTGTGAGTGGCAGCAAGGGGAAGGCCCAAGCAAGTTGCTAGGTGCTAGGAGCCTGT
CTTGGGCAGAGAGACTGAGGGCAGGGGTGTGGAACAGAGCTGTTTCCTGTCTTGCCGCATAGGCCTCTGTCGTTCTATCTTGTGTCCCTCCATC
TGTCCTAGGAGGCCCAAAGGGCTAGCCAAGTCATTAGAGCATGTCAGGATCAAAGTTTATCTCAAAGACGGGAACCCCAAATCCATAAAGAACT
GTTGTGCCTGCCATACTGTCGGTAAGACTTCTAGTTAACGAAAACCGACTTCTACTTATTTCAGCTCGGTCATCAGCATCACGCTTTTGATTTG
CGGTGCATGGACAGTGAAGGTCACTGTTGCTCACAAAGACTTGGTGGAACGCCTGAGGCCTAGGGAAGAGTCATGTACTAAAATATATCTGGAA
CCATAGAAGCAAAGGCTTCTTATAAGTTAAGTGCAGATACGCACATGCGTGGTGAGATACGCACATGCGTGGTGAGATACGCACATGCGTGGTG
TGGGGCTGGAGTGAGGGAACCACTGACTGGTGGCCTTTATGCCAGGCACACGTGGTCTTCCAGGTCAGGGGCTAGAAGCCCAAGCTGAAGGTTG
TGTGGCAGCTGGTTCCTCCTCCACGCTACCTTGGCTTGCGCTAGCCATCACCCCACCATGGGCCACAGCCATTCCTTCCTTTAAGTACTTCAGA
TCTGGATCTGGGCTCCTGCTAGTGACACCAACTTGGTCACCTTTCAAGGTCCTTTCTCCAGATCAGTTACTCTCTGAGGTTCTGGGGCTAAGAG
TCCAAATTAGAACTTTGGGGACACAGATTAATATCTGACTGCCTATATTATTTTTTTTTTCGGGGAAATGACAAAATGTCTTCAAACATAAAAT
TTACCCTTTTTCCTTTTTTTCTTTCTTACTTTGGTTTTTTGTTTGTTTGTTTTTGTTTTTCGTTTTGTTTCTTGGTCTTGGGTTTTTATTTTTT
ATTATTATTATTTTTTGTTTTGATCTTCACAGAGACAGAGGCATCCTCACTACGTTGGGCATTCCTGGTGGCCTACAGTTGGCTATCTGCCCAG
CCCAACCTGCCAGTGGCACAGGTCCTGGAAAGCAGTTGCTGTTTTTTCCTCTACTTAGGAGCAGTGACCATGGCCCAGTGGCCTGTGATCCCAT
GGTCTTTATCTCATGGGCTCAGAGCCCCTGGGGTTTCTGTGATGCTATGACATTCCTATGAGCTTACACAAACAAACACCACCCCATCCTGGGG
GTTGAAGTTCACCTTTTCCCACTGTTTTGGTTCACCTCGAAATCTGTTTCCTGGTCCCTTTGAATGGGTTTCTGTAAACTCCCTAATGGCTGCT
CTAGGCAGCTGAGAGCTTTCGTGGGGAGGTGACGTGTATGGCTGTGACCATCACTGTGAAGAAGCCAGACTCCTTCCTCACAGATGCTCTTAGT
GGACAAGTACCAGGTAGCAGGAACTTGAAGGTATCCCTGAAAGGCTGTGTTCCCACATGTCCTCCAGATCTGGGCTGGAGAATTCCCACAGTGT
GAATCTACCTCCACTGCCCAGCCTGGGCACGGTGCCTCCTCCAGGCTTGTCGATCCATCCCGGCTCAAAAGAACACACTGCAGTCGCCTTCCCA
AGGGACTCCTATTGTTCTGTAGCCATTTCACTTTAGGGATCTTGTTTGTTGTTTTCTGGGGTACCCACTCCCTCTCCTGTTCAGAGCACTCCAG
TCGGCTCTATGGGACAGGACAAAGAATGGCACCGAACTCCTGCTACAACCGAAGAAATTGTATTTGTGAAGCTAATAAGGACAGTGGAAGGGCT
TTTACTGTTTTCGGCGCGGGCTTTAACTGCATGCTGCTGCTGGAAACTGGCACCGAAGTTGAGAGAACTTGCTTGAACATCACCGACAGTGATG
ACAATGGCAGACACCTTCTGTGCAGGTCGAGCCATCTTATATGTCCCTGCTGAGCACGGCTAAGGAAATGGCCAGGAGTGACAGTGTGCAACAG
CACGCTGGCACCTGTCAGTGTGTGTGGAGGGTGCACACCAGAACACATGCCTCTGGAGATGACAGTCTCCAGTAGATTGTTCCTGGATGTAGTT
TTCAGATGGTAGCTTTTAGGGAGTTGTGTGTGTATACACATTCATGTGTGCACTTACACACACACACACACACACACACACACACACACACGGA
GGTTATAAGTCAACTTTGGGTGTCTTCTCTATCCCTCTTCTACCTTCTTTTTTGAGACAGCATCCCTTATTAAATATGAAGCACACCAGCCCAG
CGAAGCAGGTGGGCAGATGAGCTTCAAGGATTCGCTTGTTTTCCCACCCACATTACCGTGGCTACAGATGTGTTCCACAGCACCCAGCTTTTAT
GTGACATCTGGGGATCCAAGCTCAGGTCCTCACGGGAACATACACAGCAGGCATTTCACCATCATGCTACATCCACGGCCTCTTTAGAGGGCTT
TAGTATTAACCCAAAGGAACTGAAGTTTAAGTGACTGTCCCTTTAGCCATTCCGTTCTGAGCCAAAACTTCACAGAGCACCAGTGTTGTGCGTC
AGAGCTTGGGTCAGCTCTGCACAGTAGCATTTTCTTTCCTTCTAAAACAGGGTCATGGGAAGTGTGTTAATGCCCTCAGCCAGCCTGACCTCTG
GACTTGCCTATAGAAAGGCAACGTTACGTGAGGAATTCGGATAAAGACGAACTGGTCTGGGGCTAGCAGAAGCGTTTTAATGAAGAGTGTTCAT
GACACGGAGACACATTTGCTGAGTTAGGTTGTGATGGCTTGGCCATTGCTTTCTAAAGAATAGAATTCCCAAGGGGTGGGGCCGACTCAGCAAA
CAGCTTTTGCTCAGAGAGACGGCTTGGTTCAGAAGATAAGAAATGGGCACTGCGTTGAAATCAGTTTAGGTCAGAACTTGTTTCTTTCAGGGTT
TGGCTGTGATGTTGGATGCCTTGATGATACCAGTGGCTTTGATGATATATACGCACAAGTGTTAGCTTCAAAAGGTCTAAGAAGTCCTCTTGCA
TTTGAGGTTATCGGAATAGCTCAGAGTTTATCGTGAGTGGGAATAAGATACAATATGCCTGTAGATGGGTCACAATAAATCCAGCTCTCATCTT
CTCCACACTTACTTACCACGCTCATCAGGGGATTTTTGCGTTGGACAGCTGTTCAGTGAATCCAGTGACCTTGTAGCCTGGGGAAGGCACGGTG
TCAGTGAAAGATAGTTTTGGTTCCTGGTTCCTCAGGAGTGATGGCCCACCTCAGGACTCTGTTGGCCAAATTGGCCTGCCCATACTTCAGACAT
CTGCCAAAAACATCCATGTCTCTGGTTTTTGGTGGGCTCATTGGGTCCGTGATGGTCGTAGATTGGTTGCAAACTCGAGTCTAGGAAGGCCCTG
ACCTTCTCTACAGCACAGGAGGGCAAGTGAATCGGTGGCCACAGTGTGTCTTGTTCAGAGATAGGTATCAAGTGAAGCTTACTGACCGGCAAAA
GTTAGTATCGTGTGCCGAGAACCCACATAGACTGCTAGCCCCAGCTAGCAGGTGACAGGCATAGACAAGGTGTCCAGGCAGAGGCCCTGGTAGG
AAAGAGGGAGGTTGCCACAGATGAGTCCATGGTTATGCAGTCTGTGTTCTCAAGAGCTATCTGCTGTTTTCTCTTGGCTTTGTAATTGGCCTCT
CATGTCCCTGCCACAGTTTGGGGAACGCACCATTAGCCTCACCAGGTGGGGTGCCATACCCGTCTTTGTGCTGTGCTATAGGCCCCAGATATCC
CTGGAGATTTGGAGGAGGGGAGGGAGGGGTAATTTGGGACTGGAAAGAAGTGGCTGTCACCCAACCAGATCTTAAGGAGGCTTTGCAGATAGAG
AAGCACTGGGCCGGGTCAGGGCTCCACACGGCCAACTCATCACTGACAAAGACAAGGAGGTCCCTTGTGAACCTGCCCAGATGCTAACTGATAA
AGCCAGGACAGACACTCAAGATGAGTTGACTCTAAGGCCTGAACCTCTGTGATGCTCCTGATTCTTAGTTGTTCTGTGGAGTCAGCCCTGGAAT
GTGCTCTGTGCAATATGGGGGCAGCAGGGGCTTCAGATTAAACCCAACTCTCCGTGGAAAGAAGGACACCGTGGGGAGCTTCGGAGCCAGAGCC
TGAGAGCTAACTGGCCCCAGAGGACAGGACTGTCTTCTAAGGCCTTTGGGGTTGTGGGTGCTGCATATAAGACCTGGGAGGTTTGTGTATGGAA
TTCTCAATCCACTCTCCTCGTGCCTTTGCGGTGGTACAGGGTGACTGAGATGCTGGTTCATGTCTGTTCACAGTTTGATACTTGGAGGATGGGC
CTTACCTCATTTGCATCTGCCTGGAAAGTTAGCCTGGTGTATCAGTTAGGACTTTCAAGGAGGCTGTGAGAGCCGAGCTCTGTCAGTATGGGGG
ATGCTGGCCTTTGGATTTCCTCTGAGTACTGTGTAAAGAAAGCGGAACTAGTGCACTTCCCCCTTCTCGGGAGAGCAAGATGACATCAGTCTTT
GAGGCTTTGAGCTCAGGATTTTTAAGTGTAACCATCTAGACTTTAATTGTTGCCAGTCTCAGAATAACCAAAATGTGACTTTGTGTCCCCAGGA
GGGCGAGTAGACAGTGTCTGCTCTCGTAGTTCTCAGTCAGTTCTGGAAGTGTTCTGCCTCTGCAGCCTTCACCAGTCTGAAGTTTAAGACTTGC
CATCTTGCTAAGAGCCCTGGCTGGGAGGAAGTGTCCAGGGCCACATGCCCCAGTGACCACAGATGAAGCAGTCATATTTCTGCTCACATGCTTT
CATCTGAAGAAAGTTGTGAAGAGTCGTTGGTAGTTGGGGTTGCCTGTGGAGTGCTGTCTCTGCCTATGGATGAAACCCAGCTGGGAGACAGTGG
TGTGGTGTGGTGGTGCTGGTGGTGTGGATGGGTGTGTGTGCACACATGCACATGTGTTTGTATGTGCATGTACATGGATGCATAGACTTGAGGA
CATCATCAACTATTACTACCCAGGCTTCGTCCACTTTTTTCTGTGTGTGTGTGTGTGTGTGTGTGTGTGAGAGAGAGAGAGAGAGAGAGAGAGA
GAGAGAGGTGGGGGTTCTCTTACTGGTCTTGCTTGCCAGGGATCCCCAGGGATCGACCTGTTTTCTGCCTTCATGGGTCTGCCTTTATATTTTG
CCCCCTAGAATGTGGGTTCTGGGGACTAAACTCAGCTCCTAGGCTTCCAAAGTACTTTGCTAATGGTGCCATCTCCATGGTCCCCTCTCTTTTT
TTCATCTACTCTGAAATCCCTTCTGGTGACAGGAGTCTGAGAGGCAGGCATTGCTGTACTTGGAAGAATAGCCAGGGAAGCTGCAGCTAGTGTG
GGATTATGGGAACACTTACCACATCTCCTGAAGGCCATGTACACGGGACTATTACAGACCTCTGCCAGGTGAATCACCTGTAAGGCAGAGAGGA
CTTGCCTGCCGTGATAGCTTAGGCAAGACATGTGTCACCACTGACCGCACCTGGCCATTGGAAGGTTTCCTTTGGGACATGGTGATCATATGCA
CATATTCTTCAGATGCAAACATTGCCAGGAGGTTTGATATAGGAGTGGCCATGCACCGCAGCCCCGGCCCATGGCATTCCTGGATAGATTGGCC
ACCCAGCTCTGCCTCTGATTTATAAGCTGCTGTTTCCAGAACAGGATCCACTCAGGTCACCAAGATGCGCATGGTGCAGACAGAGACCTGTGCA
AATGCCTAGCTTAACCCATCCTAGGTGACTGTGGCTATGTGTCCAAAGCTCCAGGCCATCTCCAGGTAGTAGTTTTCTATGATGACCTTGTTTT
TGCTGGAACCAGACCTCTAGAGCCAAGAAGCCCTGGGCAGCCAGTGACACTGATGGCCTGAAATCCCTAAGCTAGGAACAGAGCTGTGGTGCCT
TATGTAAGTGTCAGACTGCAGTTGTCAATTCTACAGGAGGCAGGGTCTTGGCTGCATGTCTAGGATCTCCAACCCTGGGCCAAGGTGTCCAGAC
TGTTCCGTGTCTCATGATCATTAAGGAATATCTGCCTTGTGACAGCACAGGGAATCATGGCTGACGGGAAGGAGCTTGCTTCAGACAGTCATAA
ATGGCAGTGGCTTCCCTATATGTTGCTACCATGTTCAACAGACTGAACCAACTTTCAGAACTTGACCAGCAACTGTTTGGACATCAGGCTGTAG
GTCACGGCAGAAGTGTGTCATGGCAGAAGTGTCCCGTGGCTTGCGTGTGCTCTGTCCTGCGTTCACTTGTGCGACGTTTGGGTTACCGTGTCAG
TTTGTTTCGGTTTTCATTTTTCATAAGTAACGCATGGCAGGTGTGGCCTACAGCATCCTCATCATTAAAACATCAGTGATACGAATCCTGATCA
GCTGCAGCTGCTGGGCGTCACTCGGTGGCTAGAGAAAGGCACAGTGTGGAAGAGGAAGCTGGGTGCACAGGGGAAATGCAGGGTGTGCAGATGT
CAGGCCTGCCCCGGGGACAGAGAGAGCTGGAGAGGCTAGACAGCTCCTTTCCCACAACAAATCCTCCTCAGAAATATTCTCCATCTGTCCTTCC
TTCTTTCTTTCTTCTTTGTTTTCCAGTCTGAAACAGGTAAACAGGGCAGATACCCTTCTAGAGTCTGGCTAGAGACCCAGGAGCCCAGATCTCC
TGGAACCTGGAGATTCAGCCTATCTCCCGAGCTCTGTCCTCCACCCACCCAGGATGCAGGTGGTAAGAGTAACCCTCACAACGATGAGATACCC
AGGCTTGGAACGCAGTTCTGTGGCTTCAGATGCACAAGAGGCTCATGGGATATCCATGGGCCAGGGAGGTCTTAGGGTGAAGGGCAGAGTGCCC
CAAACCTGGAAGAAGCATTTCCTTGGAGATGTCCTGCACTCTCACTGGTAAGATCTTGGGAAACAGCCAATGTATGAGGTCTGTGAATCTGCTG
TTTTAACACACACATGTAGCATGCACAAACTCACATGCCTGCTGTCTGGATCCTGCAAGTTCCAGATTTGTGACTTTGACTTGGTCTCCCAAGC
CAGTCATGCACTCTGCTTTCTGTTACAGGGACTACATGCCTTTGAGGCTTTGCTATCTTCCTTGGCAAGAGTCCCCAGTGTCCCCTGTCCTGTG
CCCCAGCCATAGGACCAGTACAGGGACTAGGGTGGGTAGAGCTCAGGATAAATGGAACACTCATTATGATTGTTGCTCTTAGGCTGTGTCAGGA
CCAGGATAGCAGGCAACGTCTTGTACTGCACAGCTGTTGTGGTAGAGGCTGGACCACAAAAGGAAGAGGCCAGCAGAGGACAGCTCCTTTGTCC
CGAGCCTAGCTTTAGGTGGGAAAGTTGGGGACTGGGCCATTTTCCTTCCCTATTCCTGCTTATGCCTGTGGGTGGGGCCCACACTGAGCACCCG
TCTTCCTCGTAGATATAGAAAAGTGCCACTTTTAGTAACATGCCCAAGATACTAGTCCAACAGACAAGGATGGCTGGTCACAGTCTCTCGGGCA
GGATGGTGGTGTGCCAGAGCCCTTAAAATATTCCAAGGTGTCCTGGTTGCTGAGGCCTTGCTCCTATCCCCACCCTTTCCATCTGAAGAGGAAA
CATTTTGTTAAGGGTCAGCTTATTGGCGCCTGTGGTCCCTAAGGTCTTCCCAGGTTGCTTTGGGAAAGGTGGGAACTAAGAGACCTGGACTAGG
CAACCAGGAATCCTGTGCAGAGATGGCACTCATGTGCTGACCAAGGAAACTAGCCTGCATGCCCTGCCCCACCAGGCCAGCCCCTGGGCTGTCT
GTCCATCTTGGCTGAGGTGGCTACCAGTCCTCTTCTGTCCAGGCACTGTCTGGACCTCTAGAAAGGCCAGCCCCTCTTGGGCGAGGCAAGCATT
CCCAGGCTAAGAGTCCTGTGGTCAGGGGGCCACAGCCCAGATGTGCACTCTTGACAGTGTTGCTGGGTGGTCCTGGGCTTGGCCCTGGATGGAT
GGACAGATGAAGCCCTTGAGGCCCAAAGTTTGCAGAACCAGCCAAGAAGTGCATTGATGGCCTTCCGCAACCCTTTCTCCTTCCTCATCTCCCT
GCCTCCCTCCGTCCCTCCCTCCTCCTCCTCTTCCTTCTTCTTCTTCTTCTTCTTCTTCTTCTTCTTCTTCTTCTTCTTCTTCTTCTTCTTTTTC
TTCTTCCTCTCTCTCTCTCTCTCTCTCTCTCTCTCTCTCTCTCTCTCTCTCTCTCTCTCCTTCCCCTGTTCCTTCCTATTTGACTCTTGAAACC
CTTGGTCTTTCTTAGTTAGAGGAAGACCTTTGGCTGCTGCCCCCGTGTTGATGTAGTCGAAGGTCTTGTTTCCCTTTAACTGGCTTCCATTTTT
TTCCACATAAGCAGACTTTAGGCTTGGGTTAGGTTTGTAGTTCCATGGACCTCAGGGTGACTAAAGCATAGATGGTCTTCACGGTTTTCCTTGT
AAGATCTGCGCAGAGGGCTTTTTTAACTATGGATGGTTCAGAATCGAAAGGATTCTACCCAAACTATGTAAGCAAATCTGTCCCCTCTGAGTTC
CAGTGCACAGGAATTAATCTTGTACCTGTAGCTGGGATGACTTTCCATGAAAGTTGGTGCAGCCTGAGGACCGGTGCTTAGCTCCCCAGAACAC
ACATAAAGCTAGTTGCCATGTGTACCCATAACCCCTGAAGGAAGAAGAAAAGGAGAGATAGGCTTCTCTTTAGAAATTCATGGGCTAGCTCAAT
CCCAAATGGGCAGCAGGAACCACCAGAGAGACCCTGTCTTAAGTAGGGTGGGAATGGATGGCCAACACCTGAGATTGTCTTCACACATGAACAC
ATACACACATGCACATGCTGAGTTACAAAGGATAAACACACTCAGGATTCTTTGTTAATACAAACAGGGTTCATTACTGTGAAACCTTTTTCTT
CCTGACCCCCAAATCTCCATCCCGAGACAGGGTTTCTCTGTGTAGCCCTGGCTGTCTTGGAACTCACTCTGTAGACCAGGCTGGCCTCAAATTC
AGAGATCCACCTGCCTCTGCTTCCTGAGTGCTGGGGATTAAAGGAATACATCACCACTGCCAGACTGCCATCTGCCATTACTATTATATATATA
TATATATATATANNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNCTGGCTTGCC
CTCTGCCCCTGTGTGCTCATTCTGAGTGCCTGTTGGCTTTCTGAAGCTCATCTGCACATGTTCTGATGTGTACAAGACGGGAATTCAGCCTTCT
AAATGTATTCTGACACTGTTGCCTCACCTAGGAGCTCACACAAAGCGAGCCCTGATCTCAGGCCGGTGTTTGCGGCTCCCTAATTTGGAATTTT
CTTAGCAGTCGTTGGGCAAGGCCTTTGGGTGGATCCTTTGTCAGTCAGCATTCCCTCCCTCCTGTCTCTGCCTCTTTGCTTCCCTCTCCTCTCT
CATCCTGGCAGAAGCTGTGGGGCACTCACACCCCGGTATGGATCTTGGTGAAGGTTGTTTGACACTTCTGTGTGTCAGATTGGTGTGAGCCCTA
AGGCACGCTGGGTTCAGACTGTGGTAGCCCAGTGGTCTTTGTGTGTCTGTGGCTGCAGATGCCTATGCCTCTCTGTGGGATTCAGCGACGGCTT
TAACCCTAGAGGTCAGTACACAGAGCGAGCTTGCCCTTTCCAACACCTCCCAAGGCAGCAATGAGGCTCCTGGGCTATAAATTGCCATTCCCTG
GCCCCACCCCCACCCCCCAGAGGTAGATTAGAGTCTGGAAGGCAGGGGAAACTATGTGGCTTTGATACTCTCAGTCCTTAGCTGTCATCTGCCC
AGCCTGGCTCCGAGCTCTGGGGCAGGTAACACCTATGCTTCCTGTGGTGAGCACACTTCTGAGATGCCTGGGACCCTGTGGTCGTGAAAAGGTG
AGAGGCAGAACTCAGAACAAACAGGAAGCAAAGCTTTGTCTCGAAAGACTCTGTTACCCATGGGTGTTCTTTGAGTATCACACAGCTGAGATCG
GTGCTTGGTTCCAGTACCAAGATCCAGGTGCTTGGTACCTGTATGAAGGGATTAGGAGCTGGGCCTGGAGACACTTGTGGCCCCTGAGCCGGCT
GGCTGAGCTGGCTGAGGTGCTAATGAGGCTGTGCAGAATCTCTGCTTGGAGGAGAGAGGAAACTGGCAACTGCCAGCGACAGGAACCAGGGAAC
GTGAGCCCACTGTGGCCTCAGGTTTGGGATCTGGTTTGGAGTTGGTCACTGTGCTGTGACCTGTTCGCCACAGCATTTTCATGTTTCCTGTCAG
TCGGAGGCAGAGCCCTTTCTCATGGGAACAGTGATTCAAAACAGGAGAGCTGGTGGGTTCTCCCTGCGCTGCGCCCACATACCAAGCACTGTGG
AAGGTCTGACTCGCCCATTATCAGGAGAAGCTGCCCGTCATTCCCACGCAGCCTGTCCTCCCATCCCTCCTCCCCCAGCATGGAGGGTACATCA
GCCTGGTCCCTTCAGACCCTACAGCAGGAGGCCACCCTGCTTTGTGACCCAAGGACCCTCAGGACCCCAGAATCAAGGAAAAGACACACACACA
CCCACATGGGCACACACACATAGGTAGACAGACCCATGCACATGTGAGTGCATACACACCACACCCACGTACACACACACTTGCTCTCACACAC
ACACATGTACATGTGCGCACACACAGACATACACACACACATGTGCACACACAGACCCACACACATGGACATGCACACACACACAGACATACAC
ATGCACACACACTCACACACATAGAGACACACTCATGCTCATGCACACTCATGCTCTCTCTCATGCACACACCATTTTCTTGAGTGAATTCAGG
AAAAAAAATCTAAATGTGTTTAGAACCGCTGAATGCATGCTTCGAACAGGCTGAGAAGGGCCGTGGGTACTTGATATGAAGCTTAATGAGAGAA
AAAGAGGGCTGGCTCCCAGATCTCAACATTTAACAGGCTTTAGTCAGCACACACGGCCCAACCACAGGAGGCTCACCTCCAGTCTGCACATATG
TTTTATTCTGCGATGTGTTTCTTTTAATTATTTATTTAGTGGTTGTGTCATCATAGTGCACACGTGGAAGTCAGAGGACAACTTGCAAGAATGA
AATCTCTTCTAGCGTGTGGGGGGTCCTGGGGATCAGGTCTTCAGACGGGGCAGCAAGTGTCCATACCTCTGGCTCCATCTCTCTGGTGCTTAGC
GAGGGTTTTTTTAAAAAAAGATTTATTTTATTATTTTAAGTGTGTGTGTGTGTATGGCCATGTGCCTATGAGTGCTAGCGAATAGCTATAAAGC
CTGGTCATGCTGGCTCCTCCTAGATCTCGAGTCACTGATAGTTAGCATTCTGACAGGATGCTGGGACTCAAATCCAGCTTCTCTGGAAGAGCAG
CCAGTGCTCTGACTCACCGAACCATCTTTCAAGCCCCTGTAACGTGTCTTAGGATGTTGGTTGTATGTAGTTAACAAGTCCTCACACTCAGCTC
TGACGTGAGATTGCTCTTCATAGATGGCAGTTTCAAAACAGGTTGGGAGAAGGTGCTAGACACATGATATCTGACAGCGCTGGTGGCAATGACT
GTGCTTTGGTGTGGCCAGCCTCTGCTGGGGTGGAGGCTAAGCCAGGAAAAGTGTCCCGAGGCCTTGCAAGGTCTTCAGTAAGACACGGGACCGA
AATCAATCAGATGATTAGGGGCCGGATGCTTTCAGAGGCTTGCTTCTGGGTCAGCAGGAAGTTCACACTCAGATCGGTTCTTTACAGAGCGTCA
CTCCTCTCTGTTGGAGTCAGGTGTCCGGTGGAGGGGCAAACAGCAAGGATGAGAATGTTCTCAGGGGCGGCCTGGGGAGTGAATACAGTATACA
AGGCTGACGTTATGACACGTTGGTGCCGGGGTGGGAGACTTCCTGATACTTTTGGGAAGCACGAGGCTAACACCTCAGGAACCCCAAACTGCTG
ACCCTGTTGTCAGGACAGGGTAAAGATACAGTCCCTGATTTAATTATGTGAACATTAAATCAATGGCGTCTATAATAGCAATGGACTGGCTAAT
AATCAAAGGCCAGCCATAAAGTAATATTCGCCTAATATTTGTAGTGCTGTTATTAAACAAAGTTCTTACCAATGAAAGATGCTCAAATTTTAAC
CTTTCTTTTAAATAATCAGGATACCAAATTATATGTTTTGCCAAGACACTCCCTTTCTGTATAAAATGTGTGCACATCCACAAGTTCTCTAAAT
TGGGTCACACGTGATATAAGATTTTAAAGTGTGTAGGTAACTTCAGATTACTTCATTTATACAAATCAAAGGGTCTCCGAGGCCTGCCAGGCCA
GCCGTTTTCCAGGTTGCATTGAGTGTAACTCTTGACAAGATGGAACTGAATTGAAGATGTGTTTGAAGTCAAAACCCTGGGTCACTCCTGAGTG
CACAACTCTAGCAGGCAACCACAGCCCCTGGGTCTGATGTCAGAGACCCTGTGGCAGCAGAATCATGCTGACACCCTGCAGTCCAGACCCCAGG
GTGTTCAGCCAGGTTTCCCCCTCTAATCTAGTCCCCATCTCTGATCTAAAGTGAAGTCCTATGGGTTTGGAGAGCTGTCCGAGTTACCGTGAAA
TTTTTCAATCCGGGTTGACAATCTTAAATGTGTAAACCACCCTTCTTTTTGCCCCTTTTCATTATTCTGTGCCTGTCTGTCTCCTATCTGCCTG
TCTGTCTGTGCCACTCACTTTTTAAAGATTTTTTCCTCTAGGGGTTGAAACTAGTCCTTCTCATTGTGAAGTTTGCTCCCCCCAGCTCTGTCCA
CAGCCAGTCTTTCTGTGCAGATATAAGAAACTTGTGTGCATATATAAGAAACACTGTGAAGGTGTGGGATGTTTTACAGTGCCTCCTCCCACAA
CACCTGGGTACCCATTCTGCCCCTGGCTTTGAGGAGTCTGGCCCCACAGCCCTGGAAACACACCCTCTCCTTGAAGTAGGAGTCTGCTCAGCTC
CCATGGCGTGCCTTCCCTGGGGATGCCAGCCTCCCTTCTGGCCAACTCCTCCTCTCTGATGCCCGCCTTCCGCTCAGCAAGCTGCTGGAATCAG
GGTATATGACACACACAGGAAACGGCCCTGACAGGCCTAGCGTGAGTTTCTAAGTGCACGGCAGCCCTTCCAGAAAGCCTTACCCCTTCTGTAA
TGTTACTCTGGAGTCTCTGTGAGCACTGCGGATGGGCCAAGCCTGTGCCGTCAGCCTGCCTCTGTGCTGGGCAGATGCAGGCACTTACTATTGT
GTGTTTGTGAGTCAGATTCCTAGAGGGATGACCCAGCTTGTACCCGAATGGTAACAAACACAGGTGCATCTTGTTAGGACTGGGCTGGAAACAA
GGGACGTGGCAGTCTGGAGGGAGGAGTTCTCTTGAGTCTGCTGTAGCTCGGCACTGCAGAGGGAAGTTATTCCCAGAGCCCCATTTGGCCAAAG
GTGCCTCTCAGGGCTTTCTTTAGAGAAGGGTGATCCTTCCTGTTCCTGGTGAAGAGTATCCAGAGTCCCATTGCTGATTCTCCTGTCTGCAAGC
AGGCTATGTCTGCACCACTTCCATGGCTCTGGGAAGTAGATTAGATTAGCTTTTAAAGACTGGCTGACTTCTTTAATTCATGAAGCACTGGCAA
GCTATGACCACACACGATTGCACAGTAACCATGGCCTTATGAAGACTTTACTCCCCTACCAAGATGCTCCTGGATGGTATCGTCTGACAACTGT
GAGGGCTGATCCCCGGATGGTGCCCCAGGCACAGCATGGGCAACTCTGTCTTGGTGTACCCACACTTAAAGCTCCTCTGTTCCTTTGCAGCCCA
TCCTTGCCTGCCCTTGACTGGCAGCTCCCGTCACATTCTGGTCCATACGAGCTTCGGATCGAGGTGCAGCCCAAGTCTCACCACAGGGCTCACT
ATGAGACGGAAGGCAGCCGGGGGGCTGTGAAGGCTTCAGCTGGAGGACACCCCATTGTGCAGGTATGGCATCTGCTCTGACCAGCCAATAGCTC
ATACCTAAAACAGTTTTGGATTCACAGCTAAGGACTTTACTCAGACGTAAAACACAGTGACCTCAGAGCCTGCTATGTTCCTTGTCTTCAATAA
TGTCCCAGTGAGTCCTTCATGAACTGGAATGTCCAAAGTCAAAGTGTGTTCACCGTACCTCACTGAGGATGCATCGTCTCCGCAGGCACTGCAG
AGGCAGGGCATCAGCCACTCTTCTCCTCACAGAGGGAATGGCTGAGCAAATGCCCAAGTCAAAGGATATTTTGTGTGAATGGGTATCACTTTCA
TACTACCATCAAGTTAAAAGAGAGACAGTATGCAGGACCACTGTGAATCAAGGGCTGTCTGTGGCTACCATATAATGCTTTTCATTCCATCTAT
AATGGTTTTAAAGGTTTGCCCAGAGCAGACTTACTTCTGGAGCAGGCACCATTACACCAACTGTTCTGCAACTCTCTTTCAAAACATCTCTGCC
CCTGGAGCGGATGTGTGCTCTCCACCCACCCCACACCACACACGTGGGATTGGTGGAAGCACCAGGTCTCAGGTCTCCAGGGCAGCAGCCTCTG
CTTCTCAGTGTGCCTGCAGGTGTTAGGAGGTAGGGTCTGAGGGAGCGGGTTATGAGATGGAGTTCTGAGGGAAAGGTGGCTGTGATGGGCACAG
TCACACTTGTGGCATTTTGGGGAAGGAAAATAGAAATCCAACTAACCCAAGTCCAAGAATCTAGGAGTTGCTCGGTTCCACGAGACTGGTTGTT
TCAGCTGGTCTGCAGTAGAAGTAGGTTCTAACAGATGTGCTGGTATGTAAGTCCAAGCAGGCAAAGATATGTGAATCCTCCTTCTTCCAATGTC
CTTATGTAGGCCTCCAACAGAGGGTTGGCCTACCACGTCTGGATCTGGGACTTGCTTTGTCCCAGGCTGACCTTGAACTCAGAGATCTCCTTGT
CTCAGTCTCCTGGGATTAAAGGCAAGTTTTGCCTTGCCCGGGGCCTAAGCTTTTCATGGCCACTATGCCTCAAGATCTCCGTGTTAAGATCCAG
GTTAGAATCCTGCGTGCCCTCCATTTCTTGATTGTACTTCATTCCAGATGTAGTCAAGTTGACAGCCAGGAATAGCCATGACACTAAGTTTCCC
TGAGCGTTTAAATTCTGGGCTGTGATATAGTTTGTATCTGGTACGCTGCTCACCAATGGTGGCCAGGAGGCACACTGGAAAGTCGGAGGCCCAG
CAACGTCACAGGTCCAGCTTGGTCTTTCTCCACCTGGACATCAGTGTGACATCCCAATGGGCGGGGTGAGTTTGTCTTGGCTTCCCTTTGTCAC
ACCCGTCCCCTTGTGAGCTTTAGATCCCAGACATTACTGTTTTAAGCGCCGCCGTGAAGTGCTCACTCTCCGGCTGACTCTGGTCACATGGTGT
TATGGAGAGCTCTTTACCCCAGTATCCCAACATCATAAACCCGGGTGCCCAACCCTGTTATTTCAGGACTGATGCAAACTGGAAGGGGTACTCA
GCAGGGTACCCTGTACTTTTATTTTATTCCATTTAGGTAAATAAAGAGCTGGCAAGGGCAGGAGGCCATGGAGACAAATGGCTGTTTGCCAACG
CAGCAATCAGCCACATGGAATGAGCCGGGCGAGGTGCAGAAACGAAGCACTTTAGCAGCGCTTGTGGGTGATATTAAGGTGATATTTGTGGCCT
GTGTGCTCGATGCCAATTAATACCTACTCTGTTAGCCGCTTGAGGTGATAAAATGCTGGGGGACATCAGTACGCAGCAGCTGGCTTGACATTTT
AAGTTTCTCCCCTTCGCAGGTGGACGAGGTCAGGTAGCCTGCCTTCAGCTGTGTTGGGTAACTACCGGTATTTACCTCTCCTTTGCCCGCTGTG
GTTTTCACTGAATGAAAGTGCATTATTGCTGAATGGAACAGATTTTAATAGGAGAGGAAATAAGTCTGTTAAAGAAAGGCAGGGGAGTAAATGG
AGAGATAATGCCTGGGCGCCTCCACTTACTTGGATCTTACAGCCAAGTGGCGCTGGGTGAGAGCGGGGGGTGGGGTGGGGGTGTGTGTGTGCAA
ATACCGTGCCCTCATCAGAAAGATGGAGCTTCAGGTGCAGGTCTGTGCCATCTTGGGCTTGGTGGTTGGTGGCTGTAAGAATCCCAGCTGCTTT
GGGGCGATAAAGCCAGAAGGAGTGTGGGGAGCCCTGTGTTTGGGGGAATCTGTGTCTGTCAGCCACAGATCCCCACTGTCTTGTGTGTCAGATG
CTGAGCCGAAGTGCTGCCCCTAGAGAACCAGGTCCAGGTGTCTTGAGCTCAGGGCAACAGTGTATATCTCAGTGCAGCTGATATCCAGGAAATT
GAAGCACTTCTGAGGGATGGCAAAATCTAAAACAGTGTTTAAGAAGCACCCTCTAAATTATTTAAGGTGGCGTTAGGGGAAGGTGGCAAAACCA
GGCCAGGCCGCCGCCACTGCCACAGAACAGCCACCGCTTCTACAACACTGTTGAAGGGCAAAGGAAAACACCCCCTAGCTAAAAAAACACCCAG
TTCGGTGGCGTTTCTCATTGTGGGACATTAGCAACTCAAGGGGTTTCCTGGGATATTCTCCCAAAGTAAACTCACACCTGTGCCCGCGTGAGGA
AAACAGCTAAAACGAGGGAATTTTAGGGGATTTGTTTTCTCTTTTGGCCGGTGGACCACAAATTAGATAAAAATAGGATTTGCACGTAATTATG
AAACACACAAAAAATAGAAAACTCTCTTGGTTGCCATGAAAGTGCTGTAACCACAAAGTTCTACCTCAGGCTTCTGGGTTATTTTGATAGACCC
GCTAGTGCCTTCAGCTCTGATGTCCAACTGTCTTGCTCTTGGAGGGGCGAGGCCATGGGCCAGTTCGCATGCCCGGTGTGCGGACACTCCACAA
GCCAGTTTGTGCCCAGTAGTCTTTGTAGCATTCCTCACTGTTGAGGGCTTCTATCTCACCCGAGAGGTCTGGACTCACCGTTCAAAATGGCCAC
AGGCTCTCACTAGCCATTCTATGGTCACCAAGCTACCCAGGCTTCAAAAAGTCTGGCCTTACAGTCTGCTAGCATTTGAAAAGCCCTATCAGAA
GGCATAGCCATGGGTTATCCTGGAACCAAGGAAGGGTTGGGCAGGGCTTATCTATAGTTTTCCTCGAAGTTCCCAGGAACGAGCTACCTTGCAG
CCCCTAGGTGGACATGGTCTGCACAAAGTCCTTATGGAGATATCCTTGGGGACACGGTGGCTCCATTCAGTTAACCACTGTCTTCTGTCAGTTA
CTGAGACAGGGGCTCCTACATCTGTCTAGAGCATGCTGTGGTCTTGCTTCTGATGTCACATTGTAATGTGAGAAATGGCCTAGATCTTCCTTGA
CTATGACACTTGCCTTGGTAGAGCAGATGTCACAAATACTCTGACTACAACAGGGGCATTTGGTCAGGGCTTCTCCAGCTCTCTCCAGCAAAGT
CTTCATTGAGTTTCATCCTTGTCCAGCAAGGTCCCTCGTGACCTTGATCTGGCCTCTGGCTATCAGGTCTCTGTGTCTGTTGGGTCTTAGCCAC
CACTGACTGAGGATCCAAATTCTCCAGACGCTTGGCAGGGGATGTGATATCTACCTTGTCCTGGGCTATGGTGGCCTTACGGGCTCCTGCCTAT
AGAGGCCCCTACTGTAGCGCATGCAATGCTTTGTGGTTTGAGGATTATCTCCCATCCGGGGGGGTCATGGACTCCAGCTCTGTTTACCAATCAC
CAAAATGACCTGCCTGATAGCATCTGCGCATCACACACAGGCCCGCTCTGCTTGAATAATTAATAGGCCACTGAAATATGAACACTCAGAAGCT
GGTCAGAGCCTAACATTTAGGTTTGCAGCATAGGGCTTGCCATTTAGGACAGTGACCAGCATAGCTACCGCCCGGCCGTGAAGGACTCAGATCA
AAGAGACACAGATAGTGTCCCAGACAGGTGACCCTGGACTCTGGGTAGAAATAAGCGACATGCTCTCAGGGCACAGACAGCAGGTACCTGAGGA
TCTGTTGGAACCAGGGCAGAGTCATAGAGGAACCCATGACACGCCCTGGCTTGTGCTCCTGCACCGGGGCCAGGCAGTGGCCAGTAGGGACTCA
TGACATTAGGGACCCTTCTGAGGTTACATAGCTTGCCCCCACCCCATATAGATTCATGGCAGGGACTTTTTCTATCGTACAGTCCTTTGACAGC
TGAGGACATCCACTGGAAGATGACTTGAATTTCTCAGGTTGTTCAAATTGTCTTTGAGCTAAGCCCTTGTTATAATTACTTTTCTTTCTTGGTA
GTGTCTTTTCCCCCAGGTCCTATGAGGAGATTTAGCATGTCAAAAATACTGTGAGAAGTCAGTTCCGAGGGGTTTCTGACCTGTGGCCAGAAGG
TCCCTCGTCATTTGGTTTAGTGGCCTGGAATGCTCTGTCCCGTGCGAGCCTGCATCACCCCGGATTCCAGGTCTCATCTCTGGAGGCTAACACA
GAGCCAGGGTCCCTCAAGGTGTTCTGGTCAGAGCACCAGAGCCCAAGGGCGAGCTTGGAAAGGCAGCATTTCACTCCGGCTGGGTCTGCAAGCT
GGTAAGGCATGCATACAGCAGGGGCTAACCTGCAGGCCTGACCCCAAAGCAGGAGCATGCTGCCCAGAGTCAGCCAACCTGCATGAGGTCTGGA
TTAATTTTTTATGGATCAGCCTGGACACTGCATGGAGTCCTCTCTCCTTTCAGGACTCTTCGAGGCAAACCTCCTTTCCCTGCCTGTCATGTAC
AGGGTAACTCTCCTGTCCACTTGGATGCTGCCCCTGCCTTCTTCACCCACACTCAGCACCAAGGTCCAGTGTTCAGAAAACGTAAGGGTCTCAG
GGAATTCCATGAGGGAAGCTGCAGTCTCCCAAACACACCACAGCTTTGCTTACTGTCCATGGCAGAGCTAAGGCTCGACAGACAATCGGTAGTA
AGAGACGAGCCGTCGTTCCAGGAAGACATCTGGGGTCAGCTCTCTTAGCGGTGGGAGAGTGAAGCAGACAGAGATGGCCGTTCGCTTACACAGT
CTGCAGCAGCATCCCTGACGGCCTCTGCTAAGCCGTACTGACGTCCTCAAGGCTGGGGTTTCATGTGGCTGGGCTCTCTGTGAGCTCATTTCAG
AGCCTTCCCACTCAGCAGAGTGGGAAGCAGGGAGGGTATTATGTGGACTGTGGGAACAAAGACAAGGCAGGGCTACAAGCAGAAGCCTCCCCAA
ATGAAGATCCTGCAAGTCTGTCACCAACCCCCCCCCCCCCCAGACCTGACTCAGAAGCTATGGCTTCTTTGTTCCAGGCTAGGAAGAGTTTAAG
TGATGCTAGTCTGAGATACAGAACCTACTGGTGGTTTTCTAGTCATAGGCCCACAGAGCTCTGACTTAGTTAGGGTTCAGATGAGCTAGGTCAG
TCCGGCCCCTCCTCTACCCTACCCTCCTTGGGCACAGGTGAGATGAGGGCTGAGTATTGGAGACAAAACAAGAACCGGCATTCGTGCCAATAGC
CACGGCAAGAAGTGAGCCACATGTCTCCAGCTGGCCTGTGACCATGCATGGACCTTCTTCCTGGTAACTCTGTGGCTTTCCTTGGCCCAGAGGA
CCCATACGTATCACAAGTGTCCCTGGCTACCCATGGGCAGCTGTTCAAATGTAGAAAAGATACAAACTGTAGAGTGTTCTCTTGGGAGGCTCGG
AGGGACTGTTGTTTGCTCTCTGGGTCTGTTTGTGCAACTGGCCCTGCCTCATCATGGGAGGTGGGGTGCAGACAGACTACTGTTGTATACCTTG
CCTGGCCTAAGGGGCTTCCTTTCATCTCCTGGGGTAGCACTGGGCTCCTTGCCTATGTGGATGGCTGCAGTTTGCCCTGTAGCTGCAGGATGGC
TGCAGGCCTTGCCTAGCCAGGCCCAGAACCATGACCTCACTGGTCATGTTGGTAGATGCCCTCAGCAAGGACCTCAGGGCTGAGAGAAGAAGCA
GATGAAGGGCCGATGCCCCTCTGGTATAACACACAGGGTCTGGGGTGCCATCGTGGCATCAGTACAACAGGTAGCCTTTGTGAGCTTCAAGTGT
TTTTAGTCCTTCATCTTAGATTCCCAGCCCTGTATCCTCAGCGTTTTGGGCTCTAGTCAGACAGTAAGGGTCAGGTTCTCTTTCATACACTCCA
TGGATGCTTGATGCATGGCCACTGAGCCCTGTGGTCCACGATGTTAACAGTAAAAACCCACCAGAACGCTGACAGCCGCAGGCCGGACCCTGCA
AGGTAGAAGGAAGGGCTGGAGTTCAGGACAGCAGCACTATTCCCTGGGCGTTCCCCTACATTTCTGGGCTTGGCATCACCATGGTAGCACGTTG
TGACGTACTACATGGCACGGCTGCTCCTGAGTGGCCTCTGCCCTGTAACACTAGCCTAGAGTGTCACCACGGTCACCGCCGCGTCGGGATGATT
CCCAGGACACTGTACAGGTGGCGGCTTCCTGAGTGGCCTAGTCTATGAGGTACTGAGGGAGGCGTCTGTGTTTCGAGTGTCTGTACCTTCGTGG
CAGTTTTACAGAAGGAGTCTGCTGGCGTTGGTGCCTTTTTCCTTGGATTTGGGTGGGCAGAGAGTGGATGAACGCTGCCATCTGCAGGGCCAGC
CTCAGGGATGCTAAATGTCAGAGATGATCCGGGTTGACATGCGTGGGGGTAACTAGGCTGATTCCTGGCCATGCCAACAGTTCCTGAGAGCACT
GAGACCCCTGGGGCCTTAAAAGCTCTCTTTGCCATTGCCAGTAGGTGGGTGCTAAGATCTGTCCTACCTGCGTGTTGCTCGCATTTGTGCCTCT
CTTTCTCCCCTGGCCCCTTTCAACCTCTCAGGCTTGGTGGGAATCGGTCCCTTGCAACTGGACCATTGATCTTGTGCAGAACAGATGGACCCTG
GTTCAGCCACGTGCCCCTGGTTTAAAATTCCCACAATACCATGCCCAGGTATTGAAGGAAGGGTCCCATTCATTTTTTCAAGTAAGGGGTCGAG
GAGGTTCCAGCCCTCTCGAGTCAGCATGCAAGGCAGGAAAGTGGCCCTCTGGGAGAAGAGCAGAGGTGATGCTGGGCTGGAGACATATAGAGCC
TCCCTGAGGAGCACAGAGCAGGCCTGTGTTCCACACCCAGGTGCCGTGGCTGTGAGCAGCGATCCTTAGATGTCCACTCTGACCTTGTGGCACC
ACAGGTCATAGAACTCACATCTGATGGAGGTCAGAGGCTCAGGGATAAACTGCCAGGAAACTGGTTTCTCCTTAGCCTGGCATCTCCCCCTTCC
CCACTGCCTATCTGCCTCAGCCTTCCTCAGTCTAGGAGGAGAAGCACAGACTTTGTGAGTGAGCTGTTTTCTGGGATGGGACGGAAGCTCCCTT
TCCGGGACCCTATGAGAAAGTGAGTCTGGACTCTGACCTCCGGAATGGCACAAGATGGCCCTCGGTGCATCTGCACATGCGCAGAGCCTCAAGA
GTACGCGCGCTTCCCAGATAAAGACAGATCTAGGGTTCTCAACTCTAATAGGTCCGTTCAATTGGCAGGAGAGCCACGGAGCTCGCAGTCCTTT
TCAGAAAGCCACAGAGCGTGGCTTCCTCCTGGAAAACCTGCACCAGGACCTTTTATATGAGAAACTGTGCCAGCTGCTGGGGAGGGACAGGCTC
CAGGAGGTGGGGGTGGCTTGCAAGGCCTTTGTAAGGAAGGAAGCGCATGTTTGAGCCCAGTGCCAAGCCTTTTCCTCCGTGCTTCAGGAACATC
AGACTTTCTCCACAGGAGACTGTTCGCTGTGTTGGGGATGAAGCTTGGGTCTTTGGGGATACAGGCAGACATGCTACCACTTAAGCCCCCAAGA
GATAATTTTGAGGTAAATTAAACAGCTGATTACTAGAGGCCTGGAACAGCTTCAGGCCCCCAGCAAGGTGGAGTGACTTTGCCAGTGGAAATCA
AGTCCCCATGAAGGTGCTCTCAGTGAATGTCTGGCAGGCATCGCCAATGTATGTGGTCTCTGGCATTTATGCCCTGGTAATATTTACATGGATA
CAACCTATTTTGTTTTTATTCTATTTTTTATTTGTCTGCCTTCTTACAAAGGTAGAGGAGGTTTGTACATGAAAGACCCAAAGTTCCTACGTGC
AGTCTCTCACAGCTCCCCTGTGCTGCCCTCGGCTTCAGACTTCTGGAAGGCCTCGTGTGTACTCGTGTATCTTAGCTCTTGGGGTCTGAGGGAC
GTCACTAGACCTAGTTTTGCCTGTGCCTATAAACCTGAGTTTGATCATGGGTACAATAGTTGTCTTCATAGGAAGAGTCTTCAGAATTACATGA
TATAAATTAAGTACTGTTGTGGGCTCATGACACATATGTGCACACACAAAGTGCTCACATGCTCATAAACACGTGCTTGCCCATCCAGGCACAC
ATTCATGAAGCAAATGCACAGACTCTAAAATACATGAGTGCTCATACAGTACAAGCACAAATGCAGCACACACACACACACCATACACACACAC
ATACACACACACACCATACACACATACACACACTCACACACACTCACACACACCATACACACATACAGACACTCACACACACCATACACACACA
CCATACACACACACACTCACACACACCATACACACACACACACACACACACACACACACACACACATACACGCGAACACACACACACACACACA
CACACACGCCCATGGATGTTCATGAGATAAACCTGTACACTGTTCCCACATAGGTCATATTTATACCAATTCATAAGTGCATACATGTATAAAC
ACATGTATAGTTATACATAAATGGTTGTACACATGTCACATATAGCAGGACATGCACACACACATGCATACCTGTGGCACCCTGAAAGCCAAGG
ATTTTTTTTATTTTCAGACAGAGCTAATCACTAGCATAAATTCTGTGGGTAGCAGATTCCACTGAAGGGCAGTGATAGTAACTCCTTCCTTCTG
TCCTAGGGAGCTATGCCCAGAAGCTCAGCCTCTGCCTGTCTCGTGGGGAAGCCCTGGGGCCTTATGTCCTGCAGCCTGTACTCATTTGCTGCAA
CACAGCCGCTCACACAGTGAGAGCCTAGTAGTCAGCGTGCCCCTCCTGGTTATGCAGAACTGGACAAGCCCTGTCTGTTTGCCATCCTTCCAAG
TGCTCCTGAGTATGGAAAATTGGTTTTGTTATTGGTTTGGTTTGGTTTTGCAGTATATGTTAGGCAAGTTCCCTACCACTGACCCACGCTCCTA
CGGTCAATCTTACCTCTTCATTTTGAAATGTCAGTCTTTTCTTTGAGTTGTCCAAGCTGACCTTGAGCTCACTCTGGAACCCACTGAGCCTTTG
AATGTGTGGCCCTTCTGCCTCAGCCTACCGCGGGCTGCGATTATAGGCCTGCACCACACAGCCTAGTTCTCAGTTTGTGCTTAAACTCTCCTAC
TGAGCATCCATGAGGGGTCCTGCCCTCGATGGTCATTTCTTTCCTACCAGACAGGCACACAGTGCCATTTGGAGTGACAGACAGTTCCACAGCT
GGTAAGGTCTTCATGCTGGTTGTTCTGAGGAGGTCACCCTTACTAACAGCAGCCGCTCCCCAGGGTCTGTGCCTCTGGCTAAGGCTGGTGTGAG
AGCCCCAGTTCACCAGAATGACAGTTGGCTCCTACCCTCTCAGATTGTGGGGCAGGAAGATGGATCAGAATTAGCTCCTGTGGGTACAGTGAGG
CCTCCAGGGCACAGTGGTGAGTTCCCAAGATGCCCCTGTACCCTGCCAGGAGACCAGCACTCTGCAGTCTCTAGGAGAGGGAAATAGAACGTCC
AATAACAGAAGACTTCCCGTGTCTGGGAAGAGATGTCTCCAGGCAGCGGCGTTTGTAATGAAGAATTGGGGCTGAAAACAATCTGAGGTAATGA
GAGTCCAACACAAACTCTTAATTAACCAAGTAGAGGGAATTTAGGTTTAAAAAATAGTGAAACCCACCCTGGGTGCCATCCCCAGCCAGGAGGA
GGGAAAGTGAGACCAATGTGTGAGCCGATTTCTCTTGTCTTCTGGGATATATCACGGGATGCTAATGTTACTCGGGGTCTTAAAAGCAAATTTC
CCTTTAAGTTGATTGTTTCAAGTGTGCATTATGTCTTGGTGGAAGGATGTCACGTGACAATGTCCTTTGCTTAGTAACCAAGTCAGAATGACTC
GCTGAAAGGCAGATGGTTTTCATCAGTTAATTGGCTCTGTTAATTCAGACCTTCCTTCCACTGCGTATGAAGGTGTGCGTGTTGGCCCAACTCC
AGCACTGAATAAATTTGTCCTCCATCAGTCTGCTGGCCTGTCATCTCCCTGTGTAAGTGCTGAGGTGGCACAGGAGCTAAAAACAAACATTTTT
AATGTGCAGGAAACTGGGGGACGCTTACACCTTGCAAGTGGAATAATGTGTTTTGTCTCGTTTAGAGGAAGCAGAGAAGACTTGGGTCTGACAG
GTAACTTTATAAATGTTGTTCTGACAGACACTGTGAGAAGCCAGAGGCAGAAGGTCTGTGCAGGGGGATGTGTGAAGTAGCTTAGATTTCTTTT
CTTAAAGTGAACTTCGTGCCTTAAATAATAGATATTTACCTCCCAGGGTAAGCTTTAAATATTCCTATTTTCACTGAAATGGCATGTTTCTAAT
GCATAAAATTATACCGTATACACCGCCTTCCATGCCTAAGAAGCCTGCNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNCATAAT
TTGTCTCTGAATTTCTATCTTTATTTTTTTTGTTACATTTCATATTGTAAAACTATTCCAATCTTATATAGACTAGTGGTTTCTGAGTAGATTC
ATCACACTGCAAGAGGCTCCCAGGTGTTGAGGGGTCTGGACCAACCAACCTTAGTTGTTAAACCCTGTTTGCAGTAACTTGTGTGTTCACACAG
CTTTGTAGTCCCTGCAGATTAAGACAGTGTTAGATGTGATCACTGGCCAGAGAGTAGCAGAGGTGGCTTTCTGCGCTGATGGCCTCAGATGGGG
GATTGCAATATTCTTTCAGTCAGCCAGGCCCACAGCAAATGGGGGAGTTTGAGGGGAAATGCCCTGTGCTTTCTGGCTCCACCCACCAGCCTTA
ATCAAGGCTGCTGTTGTCTCAGGATGCCCGGGGACCTGTGGTCAGCAGGCACCCTTTACACTCACACGTGTGCCCTTGGCACAGAGCACACATA
AAAGCTGCACGGGTGAATCTGAAGCGATCTAGCTCATAGATGCTGTGACTATGGTACAAACAGGGAGCAGGTGCCTGGCCCAAGCCCAGAGAGA
AGTCGTTCCATGCTCACTTAGTATACAAAACACACGGGGTGCGTGTCTGTGAGTGCGTGTGTGTAGTCACGTACAACAACACTGCCACCTGCAC
CTGCCCCGCAGACTGTCTCTCTCTCTCACACAGGGATCCTCCTTGATACCCGTACGTGCTCCCAACCCATGATGCTCAGAGTCTCTGCTGGCTG
TGCCCTACTCATCCACGGCTCCTCACAGGACACAAACATTAATTTTAAACATTAATCAGCAGTACCTGTAATGCTGCCCAGAGGCCTGGGGAGG
CAAAGACCTCCTGGAACCACCTACTGATGTCAGGGAGTGGGTTGGCTGCATATTCAGGACACTGTAAGGGCCACCACCCCATCTTTGCAACACA
GATTCTCGTGCGTACAGTTGTGACACCTGCGTGGCGTCAGCCGCGGGGACCACTGGGCTGTTTTGATCAGCCCCTTCATCATACAGTCTGCTGT
GAGCTCTTTATTCCGCACTGCCCTGTCTGGTGTTCTCAGATGTTCTCTGGTGTCTGTCAGCTTTCCTTCTATGTCCAGAAGTGAACGGGGGTAA
GGCTCATCTCCAACCCTTAACTTCCTACCCATCAGGAATGGGCCATCCAACAGGATTATTTCCTGGGAATGCAAATTCAGAGATACAGTCCCTT
TAAGGGAATCAGATGAGACTCTGCTTCTTTCAATAACAGGGTGTGGAGAGAGAGAGATGTTTCCTGTGGATTGAGCTGTGCTGTGGGTTCTGGA
TAGGCGGGCCCAGGAGTCGCCAGGGCATTCTTACTCTCAAGTCTAAACTTCGTGTCTGTACTGAGGACTCTCTTGGTCTGGGAGGGAGTTTGAG
GGCTCACACAAAGTTGTCCCAGCTTGCCTCCTGGGCTTTGAATTGGGAAAGGAGAAGGTTTTCGCTGAAGAAAAATGATGGGATCCAGGGTCTG
CCTCAGACAGGCTCTCACTTTTGCCTTCTCAAAGCTGTCAGTGTCTGTCACCTTGTGCCAAGTCGGCAGGAAGAGGACAGAGTTCCTCCCAAGC
CTGTGTGGAGGTCTGCCAACCTCCCCGTGGAAGCGGTCTGCTTTCATCCCAGCCATCAGAGGTGAGCACTGCTCCACCTGGCCCCTTAGCTTAT
TAAGGCAACACAGCCCGGGGCCTCTGCCCCATTAACCTCTGACTAGTGGGGAACACATTTTTCTTGCTGCCGCTCAATGAACTTGAGCATTCGG
GCTGTACCTGTTTGAAGTACAGTCCTGGTCCCCAGCCCTTTCATCTTGAGGTAGGACTGGCACTGACTGTGTGTGTCTGTGTGTGCCACATGCT
GGTGGATCACAGGACATAATGAAAGCCGTGAAGTGCCCATGCCTCTCCTAATGAGCAACTGGCATGGATCAGGCCAGAGGCGGTGGCTGAGACC
TTTGCACGAGGGACATGTGAGAGGCCCCGCTGCCTTGTAAAACCCCGATACCCACGGCCCACATCAAAATCCAGCAGTAGCTAATTGCACTGTC
TGATTTTCTGGTCCCCACTTCTAGGTTTCATTGCCTTTTTCCAATGCTTTAAAATGAAAGGCACAATCCCCTACCATCCCGAAAGGCTGGAATT
CCATCGGAGAGACTAGTAGTAATGGCTGGAACCTGGTGTCCTCTGCACTCCTCCCTTTCCGGGTACCTCCCTTCCCCCCCTAAGGATCTGTGGG
GCTATTTCTTGCGGCTGAGGCTCCCTCCCCATTCCCACCACAGACCACCTTCTTTGCTGCAGGACTCTTGAAGACTGCTTGGTGCTTTCTTATG
GTGCCGTTCACCATCCTCCTTGCTAAAACCAATTACCTAGCAATCCCGCAACTCATCACAGGGCTCTGAGTTCCATGTTCCAGCTCTCCTCCTT
TCTGCCCACCTTCTGGCCAGGCCATTCCTTTGTGGTCGGCATGAGGGGCTGGGGGCTTATCCATTGGCAAAGTGGATGACAGCAATCGGAGAAG
GCTATAATTGTCATCGACTGAGTCCTGGCAGCCACCTGTGTATCCTGGCATCCTACAGTGTATCTCATCCAACCCCGGCTTCCCCCAAGGCATC
AGGGAGCTATGGGGCATTGCAAGCATGGTGCATGAGGTGGGCGGATCTCAGGATTCTAAATAGAATGATGGGGTGGGGGTAGGGCTTCTAGGAG
TGAGGCAGAGGAGGTGGGAAGGGCGGTAGCTACCAGCATGGGGCAGTTTCACTGCGTCTTATCCTGTGGGCTATATACTCTGTCCACAAGATAC
TCTGTGGTAGGGTAAGCGGTGCAACTGGCATTGATAGGACGTCTGTTCACACCTCTTTCTCTTCCTGAAGGAGAGGTACACAGGGCAGTCATGA
AAGGATGGCCTAGACACAGTTGTGTGTGTGTGTGTGTGTTGCTGTGTTTATGGTAAATGCATCTATGTTACCAATCATGAGTGAGCAACTCTCA
CTCTGCTAACTTGTGCCATAACTGAATGTAAGAAAAATAAGCCTGTCTTTGGGGGACGTGCAGGACTGATGTGGGGAAGCCAGAAAAGCCTTAC
CTCTCCAAATCTGGAGGTGAGGGCGCCATGAGCAGTCCAGCCACCTCCATCTCTCCTTTGCTCACCTGGCTTGGAAGGACATTCCTTTCTCAGA
GGCCAGTCAGAAAGCCCCACAGGTTGGGTGTGGGCTGAGATGCCATGGACCATCAGGCTTGCTAGAGTTTTGTGGTCCTTTCTCAGCAGCTTCT
CCTGGAGAGAAGTGAGCGTATGAGTTCTGCTTTGTCACTGGCACTTGTAGCATGCAAATGGTGTGAAAGCTGCCTTTGTGTGTGTGCACATGTG
TAATATTGTGTGTGTGCACATGTGTAACATTGTATGTGTGTGACTGTATACTTTTTGTATGTGATGTGTGTACAATATTTTAGGGGAGTGTGTG
CGATGGGGTGCAAGTGTACACATTTGCACATTGCCTCTAGGTGTGCCTGTGAACCCATGTGCACACTACCTGTCTTGTTACACGTGTGCACAGG
CATGTGTGAGGTGTCTATTTGTACATTGGTGCATATATGCAAACATGTGTCTGTTTGAAAATAGAACTAAGAGTGTTCAGATCCTAATCTATAT
TTTGGGGGCCTTTAGGATAATGAGGCTTTATTTATTTATTTATTCATTCCGATAGTGTCTCATTGAGTAGTCCTGGCTGTCCTGACACTCTGTA
GACCAGCCTGGCCTTGAGCTAAGAGATCTGCCTGCTTCTCCCTCCCAAGAGCTGGTACTAAAGGTGTGCGCTACTATGCCTAGCTGCATTTTAT
ACTTATAATTTCAAGAAGTACTAATATTATATCACATGAAGGTGAAGGTCTGGGCACTGGACACTGGGCACTGGGCACAGTTCTCCTTTTGAGC
CAGTCTGTTGACAAACAGTTGGTTCCCAGGATTTTATGTTCATGGTGCCTACTTACTGCGAGTCTTCTGTTTTCTTGCCAGCTACACGGTTACT
TGGAGAATGAACCTCTCACGCTACAGCTGTTCATTGGGACGGCTGACGACCGCCTGCTGAGGCCCCACGCCTTCTACCAGGTCCACCGGATCAC
GGGGAAGACTGTCTCCACCACCAGCCACGAGATCATCCTGTCCAACACCAAAGTCCTGGAGATCCCGTTGCTTCCAGAAAATAACATGCGAGCC
ATGTAAGCCAAGTGGGGGCTGTGCCCGAGGGGTGGGCTCCTGCTGCTCTGCCCAGCACCCAGTGCTTAGCCCCAGCTCCTAGGCCCCAGGCCCT
GTGGCTCTCTAGAGCCTGAAGAAACTTTACAGATATTTACGATGACAGATTTGCCTCTTTCTGGGTTCTCAGGTGCTGTGTGTTATGGCACCAA
AGGTGTCTCAGTCACACAGCTGTAAACACTGCCCCCAGGGCCCAAATTAAGTTCCTAAACACAATGCCTGTTTACTTCTGAATGCAGCCCCGTT
CATTCAACGAATAGTTGGAATAAAGTGAAAAGATAGGGCGGGCAACACCTCTGGATTGTTTTCACTCAGAGCTGACTATGCACGCGGCTGACAT
GGCCAGCATGGTGCGCAAAGCCCCTGTGGCACACGATTAACTTAGAAAGGGATTTATTAGATGCTGCCTGCACATCACAGAATATCGAGGTCAT
CCCGTGATTTGAGTGCCCTGTGTTTTGAGTGGAGAAGCTGAGAAAACACGGATTTCCATACTTCCCATGGGAGACACATTGTACTGGACCCATG
GCGAGCACAGACAGTAGCACACATAAGCAGGTCTCCAGGCAGAGGATGAGGCAGCCATGATGAAAGCACACAGACAAACGAGACCCAGATTAGA
CTGGAAGGCCTGATGTCACTCTGCGGTACTCTGTCTCTGAGGTGACTCGAAGCTGAGAGCTGTTTGCAGGAGGGGCTGTAAGGGAGCCTGGGCT
ATGCACGTAAAGACTGCAGTACATGATTGAGTTTGGGGGACAGGTGAAGTGCAGGAGATGAAGTTGGTGCAGGAGATGAGGTTGGTGCAGGGGG
CTGTGCCAAGCTGCACCTTGTGTGTCCTTTGGTAATTTCTTCGTAACTTTAAGCCTTTTAAGTCAATGACCAAAGGGAAAGCATTCTGCGTGTC
TCAGCTTCTCACCAGTCTGAGGGACTCGCCCTCCAGGGCTCCATTGCCTGCCCTTGGGTTTTCTTCCTCAGCTATATAACCCAACTTTCCTAAG
GACAGGATAGAATTAGAGAAAAGCTCTTTTATTATATCAAGATTTTTTTTTTGTAATGTATTGAACCTTTTACCAGACTATAGATTTGAATGAA
ATTATGAAGTAAAATGATTGTGAAACCCTGGAAGAAAAGTTTTACAAATCATAAAAGGTATTAAAGTAAATAAGTGAATAATTTCGGAGCCCTG
AACCACATTGGCTGGAGAGCCGCAGGACACACAGGAGGCACAGCCTTGAAGGCTCTTCCTCTCTGAGCTGCTCTGACACATCTCTGCAGGGTCC
GAGATGTCTGGTCCCAGGAGGAGACTACAGCCTGCAGGGCTACTCAGATGTGTGGGTGCTGTAATCTTTCCCATAGGCATCTCTGCGGGGAGGT
GGGGCTGGGATGCCCAGCAGGAAGGCTGAGTCTTTAGTTCTGGTCCAGCAGACCAGCTGGGCAGAGAGTCAAGCCATGGTCTTTGTTTCCTGCA
GCATCGACTGTGCTGGGATCCTGAAGCTCAGAAACTCTGATATTGAGCTGAGGAAAGGGGAGACAGACATCGGGAGGAAGAACACCAGGGTGAG
GCTGGTCTTCCGAGTTCACATCCCACAGCCCAATGGCCGGACGCTGTCTCTCCAGGTGGCCTCGAACCCTATCGAGTGTTGTAAGTAGCAGACC
ATGCTGTAACTGTTGCACGAACTGTCCCCTCGAAGGTCTGTTTCAGTAGGCTTCCTGGAAGATAGGATCCCTCTCAGGAGACCTGACTTCCACG
TCTGGCTGCGTTACGCATTCCCACACACAGCTGGGTGCTGGTTAGAATCTGACCAACGTACACAGCACTCGTGGCCTTGGCAACTATATTAGTA
AAACGCAAAGCCTGTCATGAAGCAGAGTTATTCTGACTGTATCTTTATTTATTCCCTGCTTATTTTGAGGGAAATTGAGAAGAGGAAGTTTGGA
TTTGCACCCACCAGACTGGACAAACCCAAACAATTCCTCCTCTTTATCCTTCTCTTCCGATTCTCTTTTCCCCTCCTCTTCTCTCGTTCCTACC
CAGTTCCTCTTCTCCTTTCCCCTCTGCTCTTCTCCGCAGCTTTATCTCCCATATTCTCCTTCTCTTTTATTATCTATCCCTCCTCCCCAGTTTG
TGTGAATGTGTGTGCATATATGTGCACATGTGGTGTGCATGTATGTGTGTGCATGCGTTCAGGTGTGTATGTGTGCACACATGTGTGCATGCAT
GTGTGTGTGTATGTGTGTTGTATGTCTGTACACGGCTACAGATGGAACTCATCCCTATGCATGTTAAACAAATGCTCTGCCAGAGATCCATATG
GCCTGTATCTGAATTTTGTAACTCACCACGACTGTTCATCTTCCCTGATGGTGCCAAGTTATGCCTTTAATGTGTGAGATGGCAACAGGACATG
GCTAAGGGTTCCTGGTCCTAGAAAGAAGCTGGTATGGATGAAGCTACCACCTTCCTCTGTCCTGCTTAGCTGGTTCCTTGTCACTAGAAACATG
TCAGGGGCCATCCTCTCAGCCCCCTGCACCCTGACGCCCTCCCCAATCTGGGGACTGCATTCATGTGGGGTCATCCAGACCACTGAGGGGACAG
ACAGTCTTGGCTAGCCCTTCCTCCTCATATCCCAGCACTGTCAAACTATGTCCCTTCTCCAGCTATTGTGGCTTCATCTCCTAAGAGACTCTCA
CCATGGGGAGATACACTCACTGGGGCTTTCAGTTCCATGCTGAACTCCTGAGTTCTGCAGGCCACTGTCAATGTCCCCTGGGATGTGCAGCAGG
TCCCCTTGCATCCACTAACTGATCCTTGCTTCCTCTGGAACAAGCTGAGTCTTAGTCACACACACACACACACACACACACACACACCACACAC
ACACAAGCTTGTGGCTCTGCCCTCTGTCCCTACCTGTCTCAGTCCCCTGGAGACCTGACTTTCTCAGGCTGAATCCTCTCCCCTGCCCTGGTTT
GTCTGGCTGGAAGGAGTCTGAGGAAACTCATCTGACACAGGTGCCCAGAATGTCACCCCTGCTCCTAGCCTCTGTAGTCCCCCTTGCAGGACCA
CGCTCATGAAGTTGCTGATACCCTACTGGGTCTCTCCCAGCTGCCTGTCAAGCTTTCTGCTTTGCCTCTGCACCCAGGCTCAATCTGTCACTGG
GACTCTTCCTACCCAGCTCTTCCAAGGCCCTTCCTGCCCTTCCAGGCCAGGAGCCAGCATTAGAAGTCAGAGTTGCCTATTGCTGGCAGCTTCT
GAAAAGAGTGTTCCGTTCTTAACGTGGAATCATGTCCTGATGGGTTTGCCTCAATCCTGACACCTACGGTCACTATGGTTTGAGACCCTCTACA
AAGTTCAGGGTTGGCCAGTCCCCAGAGAATCCGAAGAGTTATGTCAAATGGCAGCACCCGGGGCCCAGCACCTGAGCTCCAAGGCATCCTCCAG
GCCGCAAAGACAAGGTTTCACTCACTCTTGCCTCCTCTGACACAGCCCAGCGGTCAGCCCAGGAGCTGCCCCTCGTGGAGAAGCAGAGCACAGA
CAGCTACCCAGTCATCGGCGGGAAGAAGATGGTGCTGTCTGGCCATAACTTTCTGCAAGACTCCAAAGTCATTTTCGTGGAGAAGGCTCCAGGT
ATGTTCTTCAGGCTGGCCCGGATGAGGTCCTGTGCTCAGTGCTGGTGGGAGCTGGCTCCTGGTTGGCAAGATATGGACCCTCCCCCTGCATGAG
TGCGTTTGGTCATAGCATGGGCAATTGTCTGATAGAGGTAGCCGTCAGTCCACAGGGGCTCTGAAGAGCTCATGATCTCATCCCCTTTGAGTTT
TTGTATTGCTGAAAGTCAGGTCAAAGTTCAGCTTTCGGGTGCACAGGGCCAGACTTTTGAAAGTGTGTGTTAGGAAAGCCTATAGGCTGGTGTT
GGAAAACTCCTACTTGCTAGAGTCATCTGCTCACCTGGAGTGGTCATCAGGCCTGGCACCTGTTTTGAGCTCTGCCCAGCCCTTCATTCTGGCT
TTTCCTCTTCCCCCTCCATGAGGGAGAGCACTCCCTTGGCTTCTCCAAGAAGCCAGTCCCTTAAACAAGTGCCAGCTAGTACCACAGTGGATAT
AAGTCTGGACCTGTTGACCTTCTGTATTCACCTCATGCGAGTCACTGGGACCTTCAGTGTCACAGCCGTGTGTGTGTGTGTGTGTGTGTGTGTG
TGTGTGTATGTGTGTGTGTGAAAGAGTGTGTGTATCTGAGTGTGTGACTGTGTGTTTGTATTAATGTGTATGAGTGTGTGTGAGTATGTGAGAA
TGTGTGTGTGTATGTATGTATGTATGTATGTGAGTGTGTATGTGTGTGAATGTGCATGAATGTATGAATGTGTGTGTGTGTGTGTATGAATGAC
TGTGTATGAGTGTGTGTGAATGTATGTGTATGAGTGTGTGTATATGTGTGTGTCTGAGATCTCCATCTCCACAGACACCCCCCCATACCCCACA
AAGATCTCCATGAGAAGACAGAGCATGGCACTGCCATGCTCAACTGTAGCTTCCTGCTCACAGTAGCCCACAATGAAACTGTCTCATGGTTTGC
AGATGCCAGGGGTGAGCTTAGAAGGGCCGGTGTCCTGAAGAGCTGTACATTTTCTTTACACCTACGGCCATTGTGTCTGCATGCCATTGGGGGC
CAGGAGTGTGGCCCCAGGTCACTTCAGTTAGACATTGGTCCTGGCTGCTCTTCCTTTCTGGCTCCAACTTGGTAGAGCTGTACTGACCAGAGAG
GCTTTTGGGCTCCAGAACACAGGGAAGGCTGAGCAACAGCCGCTTCGGTGGCTGTGGGCAGGCGTGCACTGACCCGGAAGATGCCTGCCCTCAG
CTGCAGTAACTCCTCTACAAAGGGTGTATGGAGTTTGGAATTTTGGACTCAGGCCTTCTTCCAGATGGGGATTTTTTTGGGGGGTGCCATACCC
TATCCACAAGGGTATTGGGGTAGCATCTACTCCAAGCTAGTGAGCTGGAGGCTTTGCCTGCTTTTCGTTCTTACCAGCTGGCCTTTTCTCGGAG
GTGGTCAGTCCACACTTAGCCTAATCATTGCGGAGTGCCGAGGCATTCAGTGGCCTCCATAGTGGGGTCTTGATTTTGTACCATTCACAGGAAA
CATAAGCACTTCAATCAACACAGGCTTTGACATTTAACTCGGCCTGCCTCCGTAATCATGCATGTAATTGGAAAATATGTTGTAAATTATAAAG
AAAGCCCAACTAAGTGCAGTGATAAAGAGAGCAGTGCCCACACCCTTCTCCGGGGAGGTAAATTCTGAAGTCATTTGACCTTCTGAGCCTCAGG
CCTGCCTCCCTGTCAGCCAAGGAGCACTAGACATAACTAAGGCTCCGCACTCTGCACTGTAAGATCCCTGTACTCCAATGAGGGAGCCCCAGGC
AGGATCCAGATAGCTTCCCTATCTGTTTATGGTTTGTTCAAGATGGGAAGACCTACAGAAGTGCCTGCCCCACAAGGGAATTGGCCACAGCTGT
AAACTTGGTAGCTCGCACCTGCCGCAGCTGGGTCTGAGCCTGGGAACAGTGGCAAAATGCATCTTCCGGACTACGGAGTGAGCAGCCCACTGCT
GACCACAAGCCCCTGCCTTACTCTGCTGGAAGCCCCATCTTGCCTTCATTGGATTATTCCTACCATAAAGCTAAGCTCCTGAGGCTGCAGAATG
GGTCCCTGACCTACGTTCACAGGTTACAAGTGATGTCTATACGTAAGGCCTGTAATAAATGAATAACAGATAGACAGCACTGAGCGCTTTGAAA
TGTGAGAACTCCACGGTTCTGTGTCTAGACAACAATGTTTCGGGGTGTTTTTTTTATATATACATATAATTAGATTATTTGCATATGTGCTCAG
GTGTGTGCATGCAAATGATATTTTTCTGAAGGAAGGAAACCAATATCTGCATGAGTCTTCCTTTGCCTTTGGTTTTCTAAGCAAATATCGTCCC
TGACCTACCACACTGTGTGAGTCGCGACGAGCTTTTTCTAGTTGTTTCTTTCTGGCAAGCCAGAAAGCTCAATCTGAATTGCAACCACAACCGG
ACCCTCAGTTTCTGATAGCGTGCCTGGTCCAAGTTCCATTTTTAAGCTGTTGGTTACAGAAGCCAAACATTTGGATGAGAGGAGAAACAGATGT
GTGGAAGCTGTGGTCTGGGCATGTGGGGCTGGCCTTTCTGGAAGGGGCCATCCCTATGGCTCTGACTGTTGTGGACATTGTTGACTCAGGCACT
CAGGACTAGAGCCAGGGGTTTGGCTCCAATGTATCTTCCCTGTGTAATGCAGAATATCTGAGTCACAGGTACACTGTGGACCATCATCCAGTGC
TCCGGGTCACAATGTGCAATGACTCAAAGGGTCCTCTGCATCTGTTGTCAAAGAGTTTCCCTGCCAGGAGGGCTGTGGGAGTGGTTGCCGCTCT
GCTAGAGTGGCTTGGGTAGGCACGGGTAGCAGAGCCAGAAGTTAGCTCCTACACTCTACACACTGCACCCTGCCATTTCACCGAGCAGCTGAGG
AAAGTCACTGCAATGGCTTGCAGAGCTAGCTCCTCCCTGGGTCATCAACTAATGATCTCCAGGTGGGGGTGAGGGTGGTGCCACAGGGCATCAA
GCAGCATGGCCCTGGCTTATGCATAATGGTGAGGTTAGGACTTTTCTCTGGCCTCTTCCTCCTCCTGTCAGGAGTATCCATTGAGGACTGGCTG
GAATGAGACTGTCTTTCAGGGGATGAAGGTCAAAAAGAAGTGCAGCCCTGAGCATCAGGGAACACTGACTGATAACAGACATCCTTGGAGAATA
TTTTTTCCAGGAAGCAGAGGTAGTCAGTGAGGTCTGAGGGTACTCGCCACACAGGGCCACACGTCCCGTCAGATGCTCTGGCGGCGTTTTGGCC
TATCCACTCCAAGTGCCGCAGGCGTCTGGCATGGAAGATGACAGGTCAGCTCTCCCCAACACCCCCACTCCCCACCTCAAGTATGGCTTCATCT
TCAGCTCTGCAGGCTCGATGGCGTGTTTGTATGTGAGTGAGAGCACGTCGGTGTCTGAGCAGTGGGATCAGTTATCCTCCTGAGCTCAGCTCCG
TCTTCTTTGGAGACTGACCCAAGGTGAATGAAGAATAGAGGACACTGGTTTCTAAGGGAACGCCCAGCTCTACATGTTGGCATTGCAGCTAGCT
CTGTTTGAAAGAGTATTGAGGCCTATTTGGGTGTCTGTTACTGCAGTGAAGCCAAAACCGAGGTGCCCCAAACCCCAGTACGGACAGGTGACAC
CCGACCCTCCTTCCTTCATCTTCTACACTTGCTCTGCAGTCACAATGCTCTCTGGTGTCCTGCGTCAAGTCTGCCCTCAAAACACAGACCTGCT
GTGCTTTGCTACCCACAGATCTGCTTTCCTAGCTGGCCCTTCCTCCCTCCCTTCCTCCCTTCCCTCTCCTTGGGAAGGGACACAGGAGTGGATG
GGTGGGTATTGGGGCATACAAGCAGAAGGTGGCAGAGTTTGTGAAGCAAAGGCGTCACTGGGCATAGGAACGCAAGAGTGGCCGGTGGATCCCT
CTGACCTCAGGCAGGTGCATAGATGGAGCTCTCACGCCTTTACTCTCTGACTTTCCTTGTACTTGAAGAGTGGGGCAGATTGAACACTCTCGGA
AATGGGCTGCCATGGCAGCGAGAGAGAAGGTCTCAGGGTCAAAGGGCAGCCTCAGTCAGGTCTCAGGGCAGAAGAGCAGCTGAGGAGCCTCCTG
ACTATGACCGTGTGTAGCAGGAAGTGCTCTCTCAACCTAGCTAAGGGGTCAGGGAGACACCTGTCTGATATGGAGGGTGGGGCACAGACACTTC
ATTGTCGGTGTGTCTCTGGGGACACAGCTGGTCCCAGAGCTTGCCAGAGGAGAAGGTGCTAAGCTGTGGAGGAGGGACCTCTGGACAGCAGAAG
ACAGCAGGGCTGAGAGGGTTGGGGACCTAGACTCTACGTGCGGCATGTACTCGGGGTCTCCCTAGGCTTTCAGCCTGTTTGTTAATTGTTAGGA
GTTTCAAGGGACCCCTGCATGGTAGCACTTGCCCCTGACAAGAGCATAGCATAGGATAGCATAGCATAGGATAGCATAGCATAGGATAGCATAG
CATAGCATAGCATAGCATAGCATAGCATAGCATAGGATAGCATAGCATAGGATAGCATAGCATAGCATAGCATAGAATAGCATAGCATAGCATA
GCATAGCATAGCATAGCATAGCATAGCATAGCATATCCGGGAACCCTGGGTCTCCAGCCAAGTGCAGTATAGCTGACAGGTCTTTGCTCATTTC
TGCTTTTGTTGTTTGTTTGTTTTATTTTGTTTTTGAAGTGTGAGCCTATTCATGAGCACAGGCTTCAGGGCATACTGTCCCAGGACTAGGAAGC
AGTCTACAGAGAGATCAATAGCTTCAAGCAGCTTGGGTGGGGGCGCAGGTGGAGGTATTGTACAGAGTTGTATCCCTGCATGGGGCATGTGTAT
TGTGTGTGTGTGTGTGTGTGTGTGTGTGTGTCTCAGTGTGTTTATGTATAACTGAGCATAGGTGAATGTGTATGTATGTCAATGTGTCATGAGG
TATGCATCTATCAATGTATGGGCGCATGTGCCTGTTTGCATATTAATATGTGCTTATGTGATGTGACCAAGTAATCCACTGGCCCTTTGTAGAA
TGTCCCTGCCCCTTGCAGTGCTGAGCAGGGGCACCTCAAGTTTCCCGAAATTCCACCATGTGGCCGCTACAGTTAGAGACAACCAACTTTACCT
CCCGTGGGGACAGGAGTGAGATCCCCACAGTGTCCTATTGGTGGTCAGTCTCCTGCCTTGGATAATCTATGTGCCTTGGGCCATGTATACACAG
AAGCATCTTCTCAGCTACGTCCTCAAGAACTTGTGAAGTCCCCAGATGCAGCTGCCTCAGGCTCTATAACCCAACGTGTCTCGGGGTCAGCTGT
AGGAGCACACATGCCCTGAAGTCACAACCAGCTGTAGGAAAATGCTTTCTTTCAATTCCCTGGCCATGGTCTGGGGTGGGCCAGAAACGGACAG
TGTCTGCTCCCAGGCTCTGTACGCCATCAGCCCTGCGCACACATGCTAGTTTTCATCACGGGCATAAAAATACCACCGTGCTGACCTGGTCAGG
CGAGGTTCCATGTGGCTACCATGCACCCTCACTCAGCAAGATTTGGGGAGAGCACACCCTTGAAGTTCCACTTACAGACGTGCCCAGCTCTGCA
TGGTTCAGAAATCACTGACCCAGTGAGAGACATAAGCTGGGGACCGGACCCATAATCCCTTTTGCAGAAGGCATTTCTTCAAGTCTTAGAAGGA
AATAAATTTGGAAAGGCAGTGACAGACCCACCCTGGGCATTCAGAAAGTAGACTTGGCCTGAAGGCCTGCAAGGTAGGAGAGGCCTGCCTCGGA
TCCGGGTGCTGGCAGGCTGAGTCTTGCTGTGAGCTTCACGAGCAGCTAAACTCACCATCTCAAGATGGAAGCCACACAGGGTCCCTACAAAGGA
GCAGCTGGTCCTTGCCAGTCTGGTTCCTCTTCTTCCTAGAGTGTCTCAGGATGACATTCGACAGTCGCAGTCACAATCCCTTGGATGGCACCTT
TCTGCCTATTTCCTGAAGTGTATTCACAGATTTCTCACAGGGTGCAGTTCTAGCGGCTTTGCGACTGTTTGGTCTGTGCCCAGGATACTTTGAT
CTCCATGAAAGAAGTCCCTTTTCCACAAGCACTGGCACAGTGAAGGTGTGGCTGGGTGTGGCCCTGAGGTTCCAGTACTGGAAGTACTCATCAG
AGTGGCCATGTGGGAGATGCTGGGGCTTTCTGGGTGGAGTCTAGGGAGAAGGGTGAGGTAGGTGTGTGTATGTGTGTGTGTGTGTGTCTGTGTG
TGTGTGTGTGTGTGTGTGTGTGTGAGAGAGAGAGAGAGAGAGAGGGGGGGGGTGAGGAGGGAAGGAAAGGGGGGCAGGTCACTCTGCAAGAGAC
CAGAGTGCTCCTTAGCTGAGCGACCTGTTGTGAAGGGCAAGCCTGGCCCTCCGCCCTCTGGCATCCTGCCTAACGGTGGAGGCTTTTCCACATA
CTCCTACCACACGGTGCCATTGCTACCAAGCCCCCCTCCTCCCCAGGGACAGAGTGGTTGCTGAATCATGCCCTGCTTCGGGCATTCTATTTTA
GCCACAAAGAGCAAGAGAAGCCGTCAGCTCTTCCCTTCCTCACCCAGATGCCCACTGTCGCCTCTCCGAACCTTGTGGATTTCCCTCCTTGGGA
TACTATTTGTCGGTAGAGGCAGACAGTGTCTGTGCTTTGCTCTCTGGTGCCTTTCACAAAATGGCATTTTAATCCCTGTTAGTCGATGCCAGTG
CTGTTCATTTCTGTGACTGACCAGTACCCCCATGTGGCCAGGGCAGATGGGCATGGGTTGTTCTGTTCTCTGTGACTATCATGAATGAAGGTGT
TGTGGATGTTCATAGACAAGGTTCTATGCATGCCCGTGTCTTCCTCCTGGCACGTCCCTGGCTGTGAATCTGTCCTCAGCTATGCAGTGCCACC
AGGCTGTTTCCCAAAATGCTGGGGGTATGGATGGCGGGGGGGGGGTCTTGTTTTACCTTGGGGTTCTTCTGAGGGGTGCAAAGCTGCCCTCCTA
ACCTGGGAGAAGCAGGCCTCCAGGGCACCACATGCACACCAGGCTGGCGACCCTCAGAGCCCCTCTGCAGTCCAGCCCTGCAGAGCCTGTGTTC
ATCTAGCCCCGTAACACAGCCATCCCAGTGCCTCACACTGATGGGGTGTCTGGACCTGCCCCTCCCTTGAGGCTGATGGGATGCAGACTCAGGG
CTAGAAGGAGGAAGCCTCCTGCTCCACCCCTAGGAGGGGGCGAAGCACATCTCAGAGGCTTCTCCACTCCTGAATAAACTCCAGCTGCTTCCAC
GTGTGGAAACAGTGGCTCAGCCCCTACCCTGGCGTGTGACTGGCCTGTGGGCCTGTGGTGGCAGCGGCGTCCTGCAGCCAGTGGAGCGGCTTCA
GACCTCCAGGCTGGCTGTGTGGAAACCTTGGGCGGGTGTGGTGGGGAAGCCTTTCTGATGACTCACACTGCAGGGAATTCCCTAATGATTTGGA
ATATTTTTACTTCTGAACTGGAGCACAGTTTCCTACTGGGAGCGTAGGGAGCTCCTGATAAGATGGGACACAAAGGCCCTTTTAGGATGGCCGG
CTGGCTCCCTGCCTGGGAGAGGCTGAGTTCTGAACAGCACTTGGTTAACTGGCCTCTGAGCAGGGAGGAGGGGGGTCTGAACTGAGGAGTCTGT
GGACGCATCCAATGGTCTAGGGCCCCCCTCATGGCCGATACCTGATGATTCCCTCAGGAGTATGAAGGACTATCTCCACGTAATTTGCCCCCCA
CCACCCCATCCCCCGTCTCCTGCCAGAAGCAACCTGCTAGGTGTACAGTCAGAAGTCCATTCAGCCACCGTCAGAGCGAGAAGAGTGCAGCAGA
GTCACTGTTTCGTCTATTTGACAACTGCAGCTGGAGGACCTGGGTCTGGATGGGACCAGTGCTTTGAATGCCTGGCCTGGGATAAGCACAGACC
TTGGAGACCCCAAGGGACCATATTTTCAATGGGAAACAGAAGCCGATGGGTGATCTGTCTTGGTGCAATTTAGAAGACAGGAATGGAGTTCTGT
CTTGTCTGGTAGTGGTTGGCATGGGAATGCTGGATTGGAGCTGGCCAGTCTTGGCTTGGTAAGGCACAGTGGCCCCTTCTCAGCCACTCTGCTG
GGACAGGGGAACAATCCGTCACAGGCAGCACCTTCTCCCCATGGTTCTGAGCCTTGGCAGGAACTTTTGGGATGACGGGGGCCACACTACAGTT
GGCACACCTATGTACAGGCAAAGAAACTGTAGATTCTCAGGGACGAGAGGCAGAGAGCTGCCTCTCCCTTGTGGAGGGCAAATCTAACACAGCA
TGCAGCCTGACTACAGACCCAGAGCACGGTTAAAGAACCGCATGTGGGTACTTCCAGTTTGACCCTTGTGGCTATAGGTGGCCCATGTGGCTGA
GGTTGAGGGTGCTTCTCGGCTGTCATCCCTGAGCTACCAGAGCCTAGGGGTTCACTGTTTCTGACATTCCGGTGCTCTAGGGGTATCTTGTTGT
TTTCCCTGAGGGGTAAGGATAGGAATGCAGTTAGACCCCCTTCATGGGCTCACCTGATGGCATTTGCTCAAGGCCTGGGGTATCTGGGGAGCTG
GTGTGGCCTGGTGAGGCTAGGTGGCTCTTGGAGCCTCTGGTGACTCACATTTCCACCCCGTGCCTCCACCCTCCGCCCACCACCCTGTCCAGTG
CTGAGGGAAGGAGCTGGTGTTTGATTTATCAGTAATGTAGGGTTAGTGACAGACAAATGGGGTGTAAGCAGAAACTGAGCGTGTGCCTAAACCG
GCTCATCTCAGGAACTGACTTCATTTTGGAGGAGATAAAGGCCAGGATGGATAGCCGTCAACTCAGTGGCCACCCAGTTGTCCCCAGGTCATGA
GCCTGTCACCAGGACTGGGGACTCAGAGCCTGTGAGTGGCAGACACCCTTCCTCTAATCACTAGGTCCACACCTCATCGCCAGCTGAGAACAAT
ACGTTCTTTCACTGTGCCTATGAGAGCCTTTTAATATTTTTTTTTAACATGCAAACAGCCACTGGGCTAAGGTTAAAAATACCACGTGTTTTGC
ATTTCTAGGTCCTGATGTGGCATGAAATAGCCAAAGAAAGGCTTCTCTTATCTCCTCGGTGCTTCCTCTGGGCAGAGGCTGGGACTTTGCCCTG
GCTTCCCCCAAAACACATAGGAGGCTGGTGCGACTTACCAAGTGGCAGAGCCAGGCTGCGCTCAAAGCACAGTACATGTTGTGCATTGCTGGGT
TATATTTTGGGGGTGCATGTTGTGGATTGTTGTTTTTAGGGGGTGGGGTCGCCCTGCCTCTCTCTGCTTTTCTGTCCCTTGGGAGAGACTAAGG
TGTAGGAAACCTTCCCTGATGTGTGTTGTGGCAGACAAGATGAATATAAACCTGGACACAGGGTGGACCATTAGAAACACTCTTGATTATGTGG
GCCCCTCCTAAGAATATACACTGGGCATCGTCTTTGAACTCACCAAGTACACAGGGCCAGTACTCTAGAGCAGGTCAGCACATGCCATCCAGTG
TAGGCGGAATATCTAGGGGACACAGAGCACTGCCCTATGACCTCACCGTTTCTTCCCACTCTCCCCTCCATGACCCTTGATTTCTGGGTCACCT
GGAACATTTTAAAGATATACTATCCCCCAAACAGAGTGACAGATTTCTTCCTGCCCTTTGTTCCAGATGGCCACCACGTCTGGGAGATGGAAGC
AAAGACTGACCGGGACCTGTGCAAGCCAGTAAGTACTTAGAATCCTGCAGCAGGGACGAGGGAACCAGACCGGGGCTCCTGTTTGTTAAAATAA
AACAGTGCTTAAAAAAAAAAAAAAGACAGAGGCATTGCCCAAATCCCTTGGCCAAATGAGAGCCACTAGAATAGAATGGAGGCAGTAAGGAGGG
GCAGGAGGAGAGAGGCTGGAGAGAGCTTGAGGCCTCTTTATTCTGGGTGTGCTTTGCTTTTTAAAGGTTCCAGGTTCTGTCTCCTCTCACTAAT
GGTGTAGCCCCAATTGAGTGGCTACCCCCCGGGAGATCTTGGTGTTCTAAGCCTCATTCATGGGCACCCAGCACCTGCCTGGACATGCCACCCA
GCCTGTGTGACTTGCCACCACCCTCTCCAGAGAACGCCTCAGCTACCTCGCGCTCTTGTTTTGAACTTGGCGTGGACGAAAGGGCTGACCGAAC
TGGATAGCTTAGCTCAGCTGGTTGGGTTTGCTGAGGAGCGGTGGTGAATGTGTGACATCGTCTGTGGGTGACATAATTAGTCTGTCTCCCTCCC
GCGTACATACGCACCTGCTCGTGTTCCCTCTTCACCATCATGTGAGGTCATTATTGGTTGTGATTGGCTGATCTTGTCTCATATACTTGTGCTG
GCTATGTCTGCGGCTGTGACATGTTTCTGACTGTTGGTGTGGTCTTGTCATCTAAAGTGGCTGCTATTGGAGACCTCGTGGAGTCAGGGTCCTC
TTTGCTGACTGCAGTCTGTCTTCGCCATCCCTACGGTTTCCTCCCTTGCTTGCTCGGCTTTTGCAGTACTGGAAGTTGAACTAAGGACCACGCA
TACCAGGCAAATGCTCTACCATTGAGCTATAATCCCTACCTCAACAGTGTCTTTTAATTGGACATTAAGCTGTGGATGGCTAAACAAATTAGAG
TACTGTCGCTGAGTCCTGTGCGAATTCAGTCTAGTCTCAGAAGAGCATAACAAAATGCAGTGAGTAGGTTTTTGAAGACACCCAAACCCTCCCT
AACTCTGTAGCTCATTGCACAGACTTCGTCACAATAGTCTGGGCAGCGTGCTTGTGACCACAGGGCATGAGGAGCCTTTGGTGCGCTCCCAGTG
TATGGAAGTGGGATTTAAATGAATGAGAGAGCTTTTTTTGGTTTTGTTTTGTTGTGTTGTGTTGTTTTGCTTGTTTGTTTGTTTTGGTTTTTCG
AGACAGGGTTTCTCTGTATAGCCATGGCTGTCCTGGAACTCACTCTGTAGACCAGGCTGGCCTCGAACTCAGAAATCTGCCTGCCTCTGCCTTG
GGCATATGAGAAAAGGACTTTTTCTATGAACACTGGGCAGGCAGCAAGAATCACATAAATGGGGCATGCTGGTAATGGGGTCCACAGAGTGGAC
AGGCAGTCATGGCTCCCGTCAGCAGAGCAGTCACCAGGGTGTCTGCTTCAGTTGCAGCAGGCACAGGCTGCCTCCTTGTCTTCTAGCTGAGTCA
ATGAGGATGGGGGAAAAGAAATCCTTTCCTGCTTAGGTTCCTCTCAGGCACTGAGCCACTCTCAAGAAACCTTTCCAAGTACTTCTCAGAGGAA
AGGTCCTCTCTGGCCAGTGATACTGTTCATGTAGATGAAATCTTAAACATTCACTCACAGTACCCGGCTCCCTCCCTCCCACTGAAGAGATCCT
TCTCTGCCTCAGTCTTGTCTCTTGACTTCATCCAAGTTGTCCTTAAGGAAATATTGAAGGCAGTGGCACATACACATGTACATGCGCACGCGCA
CGCACACACACACACACACACACACACACACACACGGAAACATACCTGCACACACATTGCATTTATAGATATTTTAAAGATGGGTTTCTGCTCT
GTATGTGGTTATGCATCGCCCCCACTCAAGACGGTGGGTTTCCTCAGGCCTGTGCTGGCTGCAGCCTTTACAGTCTCTGACAACACTGAGAAAC
TCCCAACACTTGATCTCAGGGCCCACTTCCTCCAGCTTCCTTCCTGGTTTTGAGAGCAGAGCTCCTGGGGAAGGAAGAGGCAAGCTTGTAATAG
GATCTGTCTGTGCATAGTGTTGAAGTGCGTTGACTTGTGTCCGTATCAAGATGTGACCCTCTTTGGTCATTTGGGATAAGCAAACAAGCCAGGG
ATAGCCTATCCCTTTGGTCCTTTCTGCGTTCCTTCCACGGACCTCAGATATAGAGACCACCATGGGGTAGCCTCTTTCCCTTCACCTGACTGAA
CTCCAACGTCACAGCCAGGCCACTTAAAGCTGGGCACAACCCAGGCCTGAGTGGCCACAGTTTGGGGCTGCTTCCTGCAAAGGTGGGCATGTAG
CATAGGCACCCTTTACCCGCCCCTCCCCCACCCATCTCTTCCAAAGGAAGTGAAGGTAGATGACACAGCTTCCTCAGGTTGATGGAGCCCAAGG
GGATGGAGCCCAGAGGGGACCCTAGCCAGTCCTGCCCTGGGGCTGGCATCCAAGGGCTGTCAGTGCCAGGTTCTCTCTCTGATAAAAAGAGGCT
GGGGCTGAAGTTCTTCTGGTTTCTGTTTCTAACGCTGAGGTTTTATTATTAACATAACTCAATAAAGTTTGCTGCCATCGATTTACTACCATTA
TAACAAGCTACGTATGGTTAATTACTTTAACAAAAGAAATGGTACCATGGATTTTTCTGACACATTTCCCCTGGCCTGTTAGACTAAAACCACA
CGAGGAAAGAAACGGAATGAAGAAACTTATGGATCTGAAATCCCCAGCAGAAATCAGGTGTAGCACTCAGAAGTACTGACCTCCCTCCCTCCCT
CCAGGCAGCTGCACCATGGGGCCCGGCACCCCCAGGACTATCTCCCCCCCCAACTTGGGTGTTCCCACTCAGTTCCAAGTATTCATGGCTTGCA
TTCTTGTTTGCCCTTGAAAGAACGTGTAACAAACAAAACAAAAAAAGAAAAAAAAACTTGCATATTTCTGTTTAAAAACAGGAAAGAAAACAGC
AGCCACCTCGGTGTTCTCAACTGTTCCAAGGCCCTGGGCTGAGCTCTGCTCTGCCTTCATTATGAGGAAATTAAGGATTCTGATGGAACTCCGT
GACCATTGTCACAGTGCCACTCTGGACAATGTCCATAAGGAATTCCACCATGTGTAACCACAAAGTGCCTGCTATAAGTAGGCAGAATGCAGCC
TTTTGTCTAAAGGTCACTGGGCCTTAGATACTGGGGCAGGAGTAGGCCTTGAAGTCAGCTGTACCAGTAGCATCGAGGCAGCTCTGCTCTCTGA
GAGATGCCCATAAGGGTGTCTCTTCTTGCATTAGTTCAACCACAAGGGTCTCCCAGCCTCTAGCTATGTGAGAAATGAGGGTGCCGCTTCCCTG
AGGAGCCGGTGCACGCTACCTCCGGCCAATTCACAATCAAAACACGTGAGTAGGTCACACACAAGAGTCACCATCAGAGGCCCTCTCAGTGGCC
ATAGACAGCCGTTTGAGTGGACAGTGATGTCTGGACAAAGCGTTCCTGGTCTGTAAGGTCCTGACTGTTCCCTCTTTGCTGTTTTACCACCAGA
ATTCCCTGGTGGTTGAGATACCACCTTTCCGCAACCAGAGGATAACCAGCCCCGTCCAAGTCAGTTTCTATGTCTGCAACGGGAAACGGAAGAG
AAGCCAGTACCAGCGTTTCACGTACCTTCCTGCCAATGGTAACTCTGTCTTTCTAACCTTAAGCTCTGAGAGTGAGCTGACAGGAGGTTTTTAC
TGAGCAGCCCCCCGAGGCTATAAGAGGATGTTGTTGTAAACAAAACAAAACAAAACAAAACATACCTGTAGCCTCTTCACACCACGTGATAGCC
CTATTCACAAGACCAAGTCGCCCACCCCCTCAAAGAAAAGCGAAGCCTGGGTGTGTTTTCCTGTGACTGGTGCATGCTGGGGTCATCACTTGCT
CGCCTTTTGCAAATACAGCAGCGCGGCCAACCAAGCAGCTCTGCTGCGCTCAGGGGCTGATGCGGTCTGGGGGTGTATATCTAACCTCTGTGAG
TCTTTGGGTTAGAAGAAAGTATTTGTCAACGCAGTTTTGTAAGTAGCTTCGAAAATAAGCCTGCCGTGGTCACTGGGGAACATACATGATGTTG
TCCTCATGGTGACGCTTCTACACAGCGTGCGGTGTGTCTCCACTGAATAATGCTGTCCCCTGGTGACGTGAGACTTTCAGATGCAAGCTCTTCT
GCTCGAGTTTACTCATTTAGGGAATGGCTTCTTTCATTCAGAAGTGATCGGCTCGCCTTTTCAACTTTCTAGGGTGTTTTATTTACGAAAATAC
CGTTTTTAACTGCTCCCCACCCCGAAAGCTTCTAGAAAGGTGTGTCCCAGGCGTCCAGGGTTTCCTGTGTGGTGCAGGCCATTCTCCTGCAGCA
GGATGTATAAACAGACAGCAGAGTCGGTTGTTATCCTGAGTTCTATTGTATTTTGAGTAAGCTAGGCTATGTCAACAACCTTTTTAAATTGCTA
CTTTTTTTTTCCTCTAAAAACTTAAGATAGTCATGTAATTTAAGAGGGAAGTTATACAATAAATACTAGCCATGAAAGCAGCCATATTGCTATC
TTAGTAAAATCAAGGTGGTTTTGTTGTTGTTGTTTTGTTTTGTTTTTTGTTTTTTAAGGTTTCAAGGTTTTTTGTTTTTGAAGTGTAAAGGCAT
TTGGAACAGTTTAGACAGTACGAAAAGTTGGTATTAAAATTCTGAAACCAATTGTCTTATCAGGAAACCCCTAGAAATGCCCTTTAAAAATGAG
GACAATAGCTTTGTTGCATTCTCAAACAAGGACATCAGTGAAAGGGCAGCAACTGTCTGTGCTGTGGGTGACCCCAGAACAGCGGCCCATCCCC
CATCCCGTCTCTGCTCTTCAGATTATTTCACAGGCCTCTTCCTTTCGGGAAATAATGCACACTCTCTCTTACAAAAAAACCAAACATTTGGTCT
TTTATTTTATTTTATTTTATTTTTTGAAAGTGCAATGATTGTGTCCTACCTATACTTCAAGCATGGTTGATCTAAGATTTTTGAAAGGTCTAAA
CGTTATAAAGAAAAAAAAACATTTTCTAAAACAATGTGGAAGCATCGTAAAGATTAACTTCCCCTTGTTCAATGCACATTTGAGTCCGTGATGA
AGGTTTCATAGGGTGCTCAGTATTCGTAAAGAGCACCGTGTCAGAGGGGTCTGCGGAGCAGGCGTCCCGTGAGGCCCCAGGCTCTGGTGGCTGA
GTGTACCTCTGCCTCCCATGCTGCCCTCCCTTGGTGATGCAGTATCCAAGCATGCCAGCGGCTCCACAGTCATTTGCTCTGCACCAGTCTGCCT
CCCACAGTCCACATAGCCTCCTGCTGGAAACTCCCAGCTGCAAACCATCCTTATGTGGCTCAGGTCTTCCTTCCTCGGCCCTTCTGGCTTCACC
CATTTGCTCCAGACCCCTCTTCAGCCCTTTCATCCTGCTTCCCTGACTCTCGTCCCCCCAACTCCAAGATGGCTGCCCACCACCATTGTGGTCT
CGGGGACCAACCGTATTTCCACACCATTAGACTGTGAGCTCCTTCACAGAGACCTTGGCCACAGTAGGGTTGAATGCTAGCATTACTCATTGCC
TGATTCCTGGCTGGCTACCGACATGTGTTGTTGTTGTTGTTGTTTAAATAAATAAACACACAGATGGAGAACCCCAGCTCCTGTGTTCCGTGTT
CTGTCTGGTGCTCCCCTTCCGTGCTCCGTGGCATCTGTTGCTCTGGGGTGTATCTCTGAGAACACCGATGCTAATGAATGGATGCCTGTGTTCA
TAGCTCCCCTGGTCTCAGATTGCTGCATTTGAATCCTAGAAACTCTCCAGGGCACATGAGTGGCTCTTAATTAACCTGACTCCATGATCCTTCA
GTTTTTCTCCTTGGACACTGGCTGTGAAGTTCCGAGCCTCCCCTTGAGCCTTTCTGTATTATTAAGTGAGTTTCATGTATACTAGTATTTCACG
TATCTGTGACCAATACGGACACAGGTTCTTATGGTCATCAAATATGCTGTGTTTGAGAAAGTAGGAATAAATCCTTTTAAACCCTCCCTGTGAA
ATTCATTTTTATCTGTAGATATACAAAATCGAGACTCATTTAGTTTTCCTTCAGACTTAGGTGTGAGGATGTCTCAAGGGCGCTGCATCAGATT
CCTCCTCTCCCTTACCCTTGTGAAAGGGCCCCCACTCTTGCACAAAACCGCCCCTTGTCCCAGGCCCTTTGAGATTCAGGGGGTTGGAAGTCAA
AGTGTCCAAGGTATTTTGCTGAGTATAAGGCAGGGGGATGCCAGCTTTGTGCGTTCCTTTGTCACCTTCGCTGTCTCCTGATACCTCTACAGCA
GATTAGCTCACCTGGTTGACACCCACCCGGGGCCATCCTCCTTTCTTATTCTCAGTTAACTGTGACAGCAATCAGGTGGCAAGAAAATAGAGCA
AGTGACTGGCACCCTTGGGTTCTGGGTGGGGTGGGGGGGGTACTGAAGTCTTTCGTGTGTGGTCTGTGATGACCCTGGAAGGGTAGCTTGGCCT
CCTTCCCTCCTACTCGTGTGTGCCTAACAGGGAAAGTCTCCACTTAAAGGAAACTCCTGGGCTTCCCGAGACTCTTGTCTGCCACTAACTTAGC
TTTACTCCCTGAAAGGAGACTGTACTATCTTCTTCACTGAGCTGTGGGGGCTGGGCAGCACACCTTAAAGCCAGTGCTTCTGTACAGTGAGGGG
ACAAGTCACCGTAACCTATACGATTCTGAGAGCCTGTCACTTTGTCTGAAACTGTTTAGCTAGCTAGTTAGGAGTCCCCCGGCCTTCCCAGCCA
GGACAGGACTGTCAAGGTGGGAGTGACTCTTGTCCAGAGACCAGGAACACGTACTCCCGAGCAGATCTTGCACAACTCATTGGAAACCCTACAG
CGGGCTCTGCAGGTCTCTGGGATAGTCATCAGACACAGATGCTAAAAGCACCTGCCGACTGAGAAGAGACTTCAGAGTGCCCAGTCCCAGCCAG
GGAGGGCCTCCCGGGCTTCCTGAGACTCTCACTTGACACTAACTTTGCTTTACTCCCTAATAAGAGACTGTAATATCTTCTTCACTGAGCTGTG
GGGGCTGGGGTGGACATGGTCATAAGAAGAGGGCTATGCCAGGAATGTCTGGGTCTGAGCCCTTATTCCAACATGTCAATTATAATTCCAGAAC
GATGAATGGATATAGAATGTCTCCGGTTTGGGTTCCAAGATGAGCATCTCTGCCAGTCTGAGACCTCAGACTAGCTCTTTCTTCCAGTCACTCG
TGGCAGGAACTGGTACCTGCTCACAGGTGCAGGGGTGCTTATAAAAAGAAGAGTCAGCTTATTATAACAGCACACCCTAAAGTCAGTGTTTCTG
TACAATGAGGAAACAAGTCTCCACAAACTATAGGATTCTGAGAGCCTGTCACTTCGTCTGAAACTGTTTTAGCTGGTTAGGAGTCCCCTGGACT
TCCCATCCAGGACAGGACTGTGAGTCCAGGAACACCTACTCCCATCCCTGACCCAACCCCTAGCACTGGGCACTCGTGTGAGAACAGAGACTGA
CCTGGTGACAGAGACCAGTACATGACTATAGGACAGGAAAAGCATTGCAGTGGAACCAATGTCAGTCAAGCAACCCCCTGAGCACACAAGACCT
ATAAGGATAGGTTGCCCCAAGGGCCAGAAGTCTATGAACCTCTAACACCCCTCCCCCTCCCACACACAGCCACACTTCTTTGCATGGACTCTGG
GGTGGAGCCATGAGTGATCTGGAGAATAAAATACCATAAGGAACTTTCTTGAGCCCTTCCTGATTATTATTAGTAAGTACGTCTAAGATATATT
TCCATCTCAGAGGAATAAGAGGAACGCACCACTTCTGCCGGATTCTTGGTTGTATCACCAGAGTCCGGAATCTTAGAGACTCGTGTAGACTTCA
GCAGACGACCTGGGTCCTGCCTAGAAGCAGAGCCCCCACCTTATAATGGATCCCTGTGCCTCATGGGCTCTGTAGCTTAGTCACTGCCCTGACT
ACAACTCTACTGAAGATATCAGGCCTCAACTTGTAACTGCCTGGGTACAGAGACCGTGATGGCATGGCAGTACTGTGCACAGCCCATGGCTGTC
TGCCTGGGCACCCACTTCCTGTCCTATGAGAAGCCTGGCCCTGGAGAGAGAGGCTAAGGTCTGCCCTACTCCAAGGGTTTCTTATGGAGAAACC
ATACAACATAGTGGCTTGTTGGGATTAAAGCCAGGACAGTTTCTTCCACTTACCATCATTCAGCTTGAGCCTTTCCTTCCTTAAAGAGCCCATG
AAGGCTTCAAATGGCAGAGAACACAATGTGGTGTGTAACGGGGCAAAGTGATGTTAATTCTAGGGACCCTGCTTCTTGATTTTCTTCTTAAAAG
CTAGTGAAGACCTTGCGGAGTGAGGAGAGGTCAGGGTGGGCCAAGGAGCGCTGTCTCTGGCGCCTAAAGCTACAGGTGGTACCTACCTTCAACT
GCCAATGACCTCAGCAACCAACTGGCCATTGTAGCTGTGTGCCTTCATGTTCAAATGACACCGGTCAGTAGACATTTGGGAGAATGGCCAAGGG
CTGTCTAGACCTCTGGATCAAGGAGTCCTGGCTACTTAAACTCTCAAATCTATCTCCAGGGAGGAGGGAGTCTGTTTCTCATACCGACACTGAT
GTGCCGAAGAGAAGGAGCTAACACTTGCACCCAGGCCAGCCCTGCTGCACCACGAGGAAGGTTAGGCATCAGAAGAGGCCATTTCTGCACTATG
TGATCTCTCAGGTCTTCCCATGTGTTAGAAGGCCTCAAACTGAAGTGAACACTTGTCTGGGTGCTAATGTGACAGCTCCAAGAGATGCAGGCCT
CTCAAGCCTAAGCTGTGTCCATGTGCGTGCACACACCTTGTGCTTCTGTGGACTCTCCATTTTCACACAGGGAAGGAGATGAAGGGTAGAAGGT
TGCCTGGAGATAATTGAGACAGAGAGAGCAAACCATGTGGCACCTGGAGTGCCCGCAGAGGATGGCTGTGACTGGGACATTTGAGGAACCATGG
AATAGAAAACCTGCAATTTACAAGGGCCCGAGAGGGAGGCCAATGTCTTGTGGAGCGATCACAGAACACAATAGGCCATTAGGATGAAGGGCAG
GAAGGTAGTTTTGTTTAGAAATCTAAGGGCCATGTGCCACCAAAAACAAACAAAAGCGTGAGTCAGAGTGAGCTGAACACCCAAGGAGGCTGAG
CACGGAGGAGACATGCATGGGCGGGAGGGAGAGCAGATAGCTCCGCCTGTGATCTGCCCATAGAATTCATGCCATATGTTTGCCACCTGTGAGC
TACTCTCAGGTCCCTGGCTGCCCCACCCAGCTCTGTCCTAGGCAGTATGGCAGCCCCAGAGCCCCAGCTGCTGGAGGGGGAGTTACTGTCAAAC
CATAGAGATTGGCTTCAGCAGCTAGTGGATAGAACGCCACTGATGTGTGTTTTCTGTGTGTATGGCCTTCTGTGCCATCCACCGCCATCAAAAG
TAAATGGTGTTTATCATTCTGCACCTGGATGGGTCTCAGCCGGTTGGTTTGGGCTGTGGACTAGCGTGCACAGCCAGCAAAGCCATTCTCTTCC
TTCTCTGGGTCGGGGCCTGCTCTGTAGCTGGGAGTTAGGACCTTGTGGCACGACTGACCTCTGTGCTCACTTTGATACCTCTGGAAAGGGCATA
TTCCCAGCTCTCACCTGCTCACCACAGATGAAAAGGCCTAGCGAGGTCCTGTAAGAAAGGCCGGAGCCAACAAGTCCTTGGTGTGTGCTGTTTC
CAGTCCCCGGCGGGCTGAGCTTCCGCCTCACCCTTCAGGATTTCGTATTCTGCCTGAATGTACCTCCACGGATCCTCCCGCTGCACTGAACACA
TCCCTCTGTAGATGCTCCAGGTCTAAGCTGTCCAGGAGACAACCCTTACGCTCTCTCATCCCCAGGGAGCTGGGAACCACACATCTGCACCGCC
ATTGTGAATGCTGAATGGCAGATGTGCCCTTCCATTCACAGGCTCACAGGTCCTGTAGGATCTGAGGTCGCTTGGCAGGAAGATGTGTGAAGCC
TTTCTGGCCTGGGCTTGAGTGGCTCTCTGTGCTCTTCACCCCTGGAATCCTCTCTTGCCCCTGACTTACTGCTGGCTGTCTATGTTGATCAGGA
GACTTAGGCTTCAATGTCACTGCCATGAAGACAATCTGACTTTTGTTTCATAGGAGTTTTAGACTTTGAACAAAACCTCTTCTGTCCGGCACTG
GAAACAGCTGGTAAAATAATTCAAGCCACGACACTTGAGTTTACTTGAAAATGCAAGTGTGAAGTAACAGGGCATGGACAGAAGACATCCTCAC
TTGTACTGAGCATACGTCCTGTGAAGGCTGTGGGAAGGGGACAAGTGCCCGCCTCTGCCTGACAGCTCTCACTGTGCACAAGAAAGGGGACTGA
TAAAGGTGCTTCCCACCTGCTCCACTAGAAGGACAAGATAGATGCCATCAGCTGGCTACACATCAGTCACCAGCAACCCTAATGGTGACCTTAA
AGACCCATCTCCAGTTGTCTTGTTGGGGATTGGTACTTCAACATGAGTTCTGCGGCATCCAGGTTCTCCCACATTCTCCATATGCACAGCGTAG
AGGGTATGGTACAGGCTGTCATTGATGGACTCGCAGAGGTCTGATAAAGCAGGCTGTATCCTCACAGCTCTGCCTCAGAATCTTCACACAGGTC
CCACTGACTCATAGCTGGCAAAGCCTAGGCTGTAGGAATGAATTAACCCAGTGGTCACACGTTAGTCATACAGGCTCTGCCATGTGTCATGTGT
GATGATGCCGTGTCTGTTGGTAATGCATCTGAGTGAGGGCTGCCCACTCAGCATACAGGAGCACTCTTCACACTTAGGACTGAGCTGGGCAAGA
ACACTTAGACAGAAACCCAACATAAGTACCATCTACACAACCGTGTGGTCTCTCTTCATGCAGCCACACTGCCTTCGTGTGTCAGATGCTGGGC
AGTCCTCGGAGCAAGGGGATTGGAGGCACAGGTGTCAGATGTCGGTGAAACTTGCCTGGCAAGGTTGCTCAACTGTTTATATATTTTTAGCAAG
TTGTCATAACTCCCCCCAAATAAGATGGTTGGATGTCCGAGGTGCTGATAGCTGCTGGGACCTTGAGCTGATGCCCCAGAATGCTGCATTTACA
ATGGGAACCATGCGGTCCGTCTGCACAAAGGTCCATGGGATGTTTGACTGATCCCAGGCAAAGAAGGACAGGGCCTGTCTGCATGGCTCTGGTC
CACAGTAGACACATTCAGGCTCCCTCAAGCACAGGCACATGGCCCGTGTATCATCAGGCTCATTTTGTGGGCCTCACAAGATAGCCACATTACT
CCAGGAGAGAGTCACCTTCTTCCACTTGCCCCACTTAAAGGAAGGTCATTCTCCACCCATTCCCTGTCCTGGCAGTCAAACCTAGGCTGTCCTG
CACCCTAGGCAAGCACTCTTAAGCACTAAGCCATATTCCAGCCTAGCTAGAAGTTATAGAAGCCAGAGTGAGTCTCTGTGGCTGAGCCTTACTA
GACTTCGCCAGAAGATGTGCACTGTTAGTCTGAATGGGATCTGTCCTTGCCTATCTTTCTTATCTAGGAGATCAGACTTAAGATTCAAAGCCAA
ACTATGAATCAAGAGCTTCCATCTTTTCCTGGGTCCTAGTGAGGACCCTCTGGCTGCTCCTCTCTAATCCACCCAATGAGAGTTGGTGAAACAT
ACACCCAAATTCCTGGATAGGATGGAGGTGGCGCGAGTCAGCTGTCTACCAGCAAGCTTGGATGCAGGGCAGCAGAGGCAGCTGGGGTAGCTAT
ACAGAACAAACTACTTCTAGACTCATACTGTTGATGGCTTCAGAAGGGCCTGTGACAGACAGAGAACAGTGGAATTCCCCCAGGATCCTTCTGT
GTGCATAAACTTATAGCCCAGCCCCAGTACAGCCTCAGCACAGACTTAATCCTTAGCCACACACTGGAGAGGGCCTCAAATCAGAACCACAGAG
ACCCTGGCTCTCACCTGGACCTTGTGCACAGCAGGCAGCCTTTGGAGAGTGCCTATAAGCCATTTTAGAAGTATAGTTTCCCAGAAAGAGCTTC
CCACCTAACCTGGAAGGACAAGCCCCAGAGATGGAACTCTGTGAGGCCCAGGAGAGGAGCGTCAGCCAAGCACAACCTGCCCAGTGGCTCACAG
ATGAATGGCCTCATTGGGAGGCATCCTCACCTGGTGTTGGCATGTAGGAACAGAAGACTATTTGCTAGGCTTAAGTCTCAAGTCTAGAGTCAAG
GAATGCTCTTTAAGGCTCCTTGGCCCATAGACTATCATTGCAGGACCTGCAGGAGATGGGACGGAGAAGGAGCCTTCCCCCATCACACAACCGA
GCATGTGCCATTGACGGTGTCAGCCACACTTGGCTGGAGAAATCCTGGAGGACAGTGAGCCCCAACCTGGGACACAAACTTTGAGGCGACTCCA
GGTCAAATCCCTCAGCCGTCCTTAGACTCTTCGAGGAGTGGGCTCACCACACCAGCACTTGTATGTGATCCCTGCAAGGCGCTGCCGTGTACAA
GGTTGCTGGACTACGGTGACTGCTGTGGCTGTCAGGTGCACACAGTGCCGGGCATGCACGATGACACACATCTCCCATGGCCGCACAGACCCAG
CATTGTGCTGTGCTGGGTGTGAGGATCAGTCACAGCAGGAGCCAGCAGCCCACCTTCTTCATGAGGGCTTGCTTTGTCTCTGACAGCTGGCCCA
AAAGATTGCCGTTAATCCTTCATGTGCTCGCTTTCCATAATAAACTCTGTATAATAAACTCACGACTGGGCTCATGTGCTGTTTTCAATGTGCT
AGCATTGCGGTGAGCTCTGNNNNNNNNNNNNNNNNNNNNCCCAGTCTAGCACGGAGACATTGCGTCACACCCCCTGCCTCCCTCTGGCGCAGGA
GACAGACCAGCGCCTGGGGCTTCTGTGGAGCCCAGGTCCCATTTCCTGACTAATAGGAAACAAGAGGCCCCATCCTACAAGGCTCTCAGCTCCA
CATGAGAAATGCAGACACAGGCAGTCTTGAGTTGTATAGCCCTAGGGTTTCTGTAGACAGACACTGTGAGGGGTGGCATATAGCTTCAGCTGAG
GAGACACTTACAGGGAACCTCAGAGAAGGGCCATCAGAATCCGATTCTTGGATCACTCCTCCTCTGCCAGATGGACAAGCTGCTCTGTCTCCAA
GCGTTCCTCAGCTGTGCTGAGTGACCATCCAGTGATCAGGGGCCTCTGTCTTTGAGTGGAACTGCCTCCGTCAGGGGTGGAATCCACATCAAGT
GTGAGACTGTGACAGGGTCCCCTGCACAGGAAGCAGAGGCTTGCCACCTTGGAGTCATCAGGTGGCAGGAGACATACCCTGTGTCTTCTTTCCC
TTTGCGCTGACCTGTGTGACACTGCCCCCTCCCAAGCACTGAGCTCTGAAGGTGAGGGACCTGAGCATAAGATAGTGACATTGGGCTGGCAATG
TTTAAGTCTCCGTCCCCATCTATCCAATCAAACAGGCTTCCTCCCCAAGAGTGGCAGCGTGGTATCCTTGAGTGGAGCTGCTGCCTCAGTCTCA
GATAACCAGACAGAAGGAGCCAGAGGATGGAGTGGAAAGGGCTAGCCAGGACACAACAGAGGCTGCGCAGGGTCTGCCCCAAACCCCAATCCTG
GTTCATTTTGTACCTCCTGAGAAAGAAACTTGGAAGTCCAACTTAGACCATCAGGACTCATGCCCTCGTGAATGTGCAGAACTTCAATGCACTC
AGCTGGCCCAGGTGACCATTCAAATGCCAGGAAGAGTCTGAGGTGGTTAGCAAGATGGGCCGGGGTACTTTAAACAGGTCCTAACAACAGAATC
TACAGGGTTCCCCAGAGCAGCCACCACACCCACTCCCTGTCTGGCCTCTGGATTGCTGGCCCAGGTCCAACCAACGCTCCAAGTCCCTGTCCCT
GTGTGGCAGGTAAATTACATTCTCTGTCATTTACTGGGCAGCTGTGTTTGGGACTGTGCCTTTCATGTCATGCTGGGTAGTTAGGGCTGGGTTT
TACAGATCTTATAGGCTGCATCGTGGGAGGGTTGCGTTGGCCTTGGGACCTGGCTGGGGACCCAGAGCAGCTCAGCCTGGAAATAATTCACCTT
CCAGTCATGGACCTTTTTTTTTTTTTTTTTTTAATCACAAGCTCTGAAAATGACCACTCTCATTTAGAACTGAACCTTCACACACAGATCCTGG
CTTTGAGCAGAGCGACTCATACCCAGGATTCTTCTCGGTGTAGTTTTGTTCTTAGCAACATTGCTGCCTATGTCTTTCTGGTCAGGCCTGTGAT
CCCAGAAACTGGCTGGACACGGTTCTGCCCCAGGTGCCTCTCAGGGTCTGACTTCTACCGGGTTCTTGCCAATTCTGGATTTACAGTGTGGTTG
AGCCAGCTGGACTGATACTGGATGGGGGTCGTTTCTGAAGCATGGCTTACCCCCACAGAGCCGTCTAGACGGGCTCGTGCCTGGAGCTGAGTTA
GTACCTGTATAACACATTTCATTGTAAGCTGTAGAACCCAGATGGCCAGGGGCTTCCTGTTCCTCGCAGCTCCTGGGAGATACAGCCCTCAGGA
AGGGCGGGACCCAGAGCTGATCTCATATATTCAACTTTCTAATGTCAGAGGGATGGGTTTCCCTTCCTTCCGTTTTTGTTAAATGCCAGGATGT
CCACAGTCCTTCTCATTATTAGCAGAGCATCCCGTCCCACAAAGCGATCTGGGGACCTGCCCAGAGAGGAGCCTGCTACCTGCCTGCACCTATG
ATTCCCACGGCTTAGGCAACAGGACAGATAGGGTGGTTGGTGAACTGAACTAGGTCTTCACTGGGAGGCTTTTCTCAGAGGCGCCGTGTGCATG
GCGTACTTGCCTTGGGGCTCCTATTCCTCCTTGTTTC
MOUSE SEQUENCE—mRNA
GGCCCGACATGACGGGGCTGGAGCAGGACCCGGAGTTCGACTTCGATTTCCTCTTCGAGTTCGATCAGAGCGGCGGGGGCGCCGCGGCCGCAGA
ACACTACAGTTATGTGTCCCCTAGTGTCACCTCGACCCTGCCCCTTCCCACAGCACACTCTGCCTTGCCAGCAGCATGCCACGACCTCCAGACG
TCCACCCCGGGTATCTCAGCTGTTCCTTCAGCCAATCATCCCCCCAGTTACGGAGGGGCTGTGGACAGCGGGCCTTCGGGATACTTCCTGTCCT
CTGGCAACACCAGACCCAACGGGGCCCCGACTCTGGAGAGTCCGAGAATCGAGATCACCTCCTACCTGGGCCTACACCATGGCAGCGGCCAGTT
TTTCCACGACGTGGAGGTGGAAGACGTACTTCCTAGCTGCAAGCGCTCACCGTCTACAGCAACCCTGCACCTGCCCAGCCTGGAAGCCTACAGA
GACCCCTCCTGCCTGAGCCCAGCCAGCAGTCTCTCCTCCAGAAGCTGTAACTCTGAGGCCTCCTCCTACGAGTCCAACTACTCCTACCCATACG
CGTCCCCCCAGACCTCTCCGTGGCAGTCACCCTGCGTGTCTCCCAAGACCACGGACCCGGAGGAGGGTTTTCCCCGAAGCCTGGGTGCCTGCCA
CCTGCTAGGATCGCCCAGGCACTCCCCATCCACCTCTCCTCGGGCAAGCATCACGGAGGAGAGCTGGCTCGGTGCCCGCGGCTCCCGGCCCACG
TCCCCCTGCAACAAGCGCAAGTACAGTCTCAATGGCCGGCAGCCCTCCTGCTCACCCCACCACTCACCCACACCATCCCCCCATGGCTCCCCTC
GGGTCAGTGTGACCGAAGATACCTGGCTCGGTAACACCACCCAGTATACCAGCTCTGCCATTGTGGCAGCCATCAACGCCCTGACCACCGATAG
CACTCTGGACCTGGGTGATGGGGTCCCTATCAAGTCTCGAAAGACAGCACTGGAGCATGCGCCCTCTGTGGCCCTCAAAGTAGAGCCAGCTGGG
GAAGACCTGGGCACCACTCCACCCACTTCTGACTTCCCACCCGAGGAGTACACCTTCCAGCACCTTCGGAAGGGTGCCTTTTGCGAGCAGTATC
TGTCGGTGCCACAGGCCTCGTATCAGTGGGCGAAGCCCAAGTCTCTTTCCCCGACATCATATATGAGCCCATCCTTGCCTGCCCTTGACTGGCA
GCTCCCGTCACATTCTGGTCCATACGAGCTTCGGATCGAGGTGCAGCCCAAGTCTCACCACAGGGCTCACTATGAGACGGAAGGCAGCCGGGGG
GCTGTGAAGGCTTCAGCTGGAGGACACCCCATTGTGCAGCTACACGGTTACTTGGAGAATGAACCTCTCACGCTACAGCTGTTCATTGGGACGG
CTGACGACCGCCTGCTGAGGCCCCACGCCTTCTACCAGGTCCACCGGATCACGGGGAAGACTGTCTCCACCACCAGCCACGAGATCATCCTGTC
CAACACCAAAGTCCTGGAGATCCCGTTGCTTCCAGAAAATAACATGCGAGCCATCATCGACTGTGCTGGGATCCTGAAGCTCAGAAACTCTGAT
ATTGAGCTGAGGAAAGGGGAGACAGACATCGGGAGGAAGAACACCAGGGTGAGGCTGGTCTTCCGAGTTCACATCCCACAGCCCAATGGCCGGA
CGCTGTCTCTCCAGGTGGCCTCGAACCCTATCGAGTGTTCCCAGCGGTCAGCCCAGGAGCTGCCCCTCGTGGAGAAGCAGAGCACAGACAGCTA
CCCAGTCATCGGCGGGAAGAAGATGGTGCTGTCTGGCCATAACTTTCTGCAAGACTCCAAAGTCATTTTCGTGGAGAAGGCTCCAGATGGCCAC
CACGTCTGGGAGATGGAAGCAAAGACTGACCGGGACCTGTGCAAGCCAAATTCCCTGGTGGTTGAGATACCACCTTTCCGCAACCAGAGGATAA
CCAGCCCCGCCCAAGTCAGTTTCTATGTCTGCAACGGGAAACGGAAGAGAAGCCAGTACCAGCGTTTCACGTACCTTCCTGCCAATGGTAACTC
TGTCTTTCTAACCTTAAGCTCTGAGAGTGAGCTGAGAGGAGGTTTTTACTGAGCAGCCCCCCGAGGCTATAAGAGGATGTTGTTGTAAACAAAA
CAAAACAAAACAAAACATACCTGTAGCCTCTTCACACCACGTGATAGCCCTATTCACAAGACCAAGTCGCCCACCCCCTCAAAGAAAAGCGAAG
CCTGGGTGTGTTTTCCTGTGACTGGTGCATGCTGGGGTCATCACTTGCTCGCCTTTTGCAAATACAGCAGCGCGGCCAACCAAGCAGCTCTGCT
GCGCTCAGGGGCTGATGCGGTCTGGGGGTGTATATCTAACCTCTGTGAGTCTTTGGGTTAGAAGAAAGTATTTGTCAACGCAGTTTTGTAAGTA
GCTTCGAAAATAAGCCTGCCGTGGTCACTGGGGAACATACATGATGTTGTCCTCATGGTGACGCTTCTACACAGCGTGCGGTGTGTCTCCACTG
AATAATGCTGTCCCCTGGTGACGTGAGACTTTCAGATGGAAGCTCTTCTGCTCGAGTTTACTCATTTAGGGAATGGCTTCTTTCATTCAGAAGT
GATCGGCTCGCCTTTTCAACTTTCTAGGGTGTTTTATTTACGAAAATACCGTTTTTAACTGCTCCCCGCCCCGCAAGCTTCTAGAAAGGTGTGT
CCCAGGCGTCCAGGGTTTCCTGTGTGGTGCAGGCCATTCTCCTGCAGCAGGATGTATAAACAGAGAGCAGAGTCGGTTGTTATCCTGAGTTCTA
TTGTATTTTGAGTAAGCTAGGCTATGTCAACAACCTTTTTAAATTGCTACTTTTTTTTTTCCTCTAAAAACTTAAGATAGTCATGTAATTTAAG
AGGGAAGTTATACAATAAATACTAGCCATGAAAGCAGCCATATTGCTATCTTAGTAAAATCAAGGTGGTTTTGTTGTTGTTATTTTGTTTTGTT
TTTTGTTTTTTAAGGTTTCAAGGTTTTTTGTTTTTGAAGTGTAAAGGCATTTGGAACAGTTTAGACAGTACGAAAAGTTGGTATTAAAATTCTG
AAACCAATTGTCTTATCAGGAAACCCCTAGAAATGCCCTTTAAAAATGAGGACAATAGCTTTGTTGCATTCTCAAACAAGGACATCAGTGAAAG
GGCAGCAACTGTCTGTGCTGTGGGTGACCCCAGAACAGCGGCCCATCCCCCATCCCGTCTCTGCTCTTCAGATTATTTCACAGGCCTCTTCCTT
TCGGGAAATAATGCACACTCTCTCTTACAAAAAAACCAAACATTTGGTCTTTTATTTTATTTTATTTTATTTTTTGAAAGTGCAATGATTGTGT
CCTACCTATACTTCAAGCATGGTTGATCTAAGATTTTTGAAAGGTCTAAAC
MOUSE SEQUENCE—CODING
ATGACGGGGCTGGAGCAGGACCCGGAGTTCGACTTCGATTTCCTCTTCGAGTTCGATCAGAGCGGCGGGGGCGCCGCGGCCGCAGAACACTACA
GTTATGTGTCCCCTAGTGTCACCTCGACCCTGCCCCTTCCCACAGCACACTCTGCCTTGCCAGCAGCATGCCACGACCTCCAGACGTCCACCCC
GGGTATCTCAGCTGTTCCTTCAGCCAATCATCCCCCCAGTTACGGAGGGGCTGTGGACAGCGGGCCTTCGGGATACTTCCTGTCCTCTGGCAAC
ACCAGACCCAACGGGGCCCCGACTCTGGAGAGTCCGAGAATCGAGATCACCTCCTACCTGGGCCTACACCATGGCAGCGGCCAGTTTTTCCACG
ACGTGGAGGTGGAAGACGTACTTCCTAGCTGCAAGCGCTCACCGTCTACAGCAACCCTGCACCTGCCCAGCCTGGAAGCCTACAGAGACCCCTC
CTGCCTGAGCCCAGCCAGCAGTCTCTCCTCCAGAAGCTGTAACTCTGAGGCCTCCTCCTACGAGTCCAACTACTCCTACCCATACGCGTCCCCC
CAGACCTCTCCGTGGCAGTCACCCTGCGTGTCTCCCAAGACCACGGACCCGGAGGAGGGTTTTCCCCGAAGCCTGGGTGCCTGCCACCTGCTAG
GATCGCCCAGGCACTCCCCATCCACCTCTCCTCGGGCAAGCATCACGGAGGAGAGCTGGCTCGGTGCCCGCGGCTCCCGGCCCACGTCCCCCTG
CAACAAGCGCAAGTACAGTCTCAATGGCCGGCAGCCCTCCTGCTCACCCCACCACTCACCCACACCATCCCCCCATGGCTCCCCTCGGGTCAGT
GTGACCGAAGATACCTGGCTCGGTAACACCACCCAGTATACCAGCTCTGCCATTGTGGCAGCCATCAACGCCCTGACCACCGATAGCACTCTGG
ACCTGGGTGATGGGGTCCCTATCAAGTCTCGAAAGACAGCACTGGAGCATGCGCCCTCTGTGGCCCTCAAAGTAGAGCCAGCTGGGGAAGACCT
GGGCACCACTCCACCCACTTCTGACTTCCCACCCGAGGAGTACACCTTCCAGCACCTTCGGAAGGGTGCCTTTTGCGAGCAGTATCTGTCGGTG
CCACAGGCCTCGTATCAGTGGGCGAAGCCCAAGTCTCTTTCCCCGACATCATATATGAGCCCATCCTTGCCTGCCCTTGACTGGCAGCTCCCGT
CACATTCTGGTCCATACGAGCTTCGGATCGAGGTGCAGCCCAAGTCTCACCACAGGGCTCACTATGAGACGGAAGGCAGCCGGGGGGCTGTGAA
GGCTTCAGCTGGAGGACACCCCATTGTGCAGCTACACGGTTACTTGGAGAATGAACCTCTCACGCTACAGCTGTTCATTGGGACGGCTGACGAC
CGCCTGCTGAGGCCCCACGCCTTCTACCAGGTCCACCGGATCACGGGGAAGACTGTCTCCACCACCAGCCACGAGATCATCCTGTCCAACACCA
AAGTCCTGGAGATCCCGTTGCTTCCAGAAAATAACATGCGAGCCATCATCGACTGTGCTGGGATCCTGAAGCTCAGAAACTCTGATATTGAGCT
GAGGAAAGGGGAGACAGACATCGGGAGGAAGAACACCAGGGTGAGGCTGGTCTTCCGAGTTCACATCCCACAGCCCAATGGCCGGACGCTGTCT
CTCCAGGTGGCCTCGAACCCTATCGAGTGTTCCCAGCGGTCAGCCCAGGAGCTGCCCCTCGTGGAGAAGCAGAGCACAGACAGCTACCCAGTCA
TCGGCGGGAAGAAGATGGTGCTGTCTGGCCATAACTTTCTGCAAGACTCCAAAGTCATTTTCGTGGAGAAGGCTCCAGATGGCCACCACGTCTG
GGAGATGGAAGCAAAGACTGACCGGGACCTGTGCAAGCCAAATTCCCTGGTGGTTGAGATACCACCTTTCCGCAACCAGAGGATAACCAGCCCC
GCCCAAGTCAGTTTCTATGTCTGCAACGGGAAACGGAAGAGAAGCCAGTACCAGCGTTTCACGTACCTTCCTGCCAATGGTAACTCTGTCTTTC
TAACCTTAAGCTCTGAGAGTGAGCTGAGAGGAGGTTTTTACTGA
HUMAN SEQUENCE—GENOMIC
AAGGCCCTGACCGGTTTATAAAGAGCACCCATCTTCTCCGGCACCAGACGGCCCCAGCCTGGCATCTCGATGAGTGAAGCGTAATGAGCCAGAA
TTGCAGGATTAAGCTTCACTTGAAACTCACTCCGGTTCAAAACTTAATGGAAAAAATGCCTGGCAAAACTTGTTTGGAGCTTTATTGAACCAAA
ACACTTTAGAAACCGTTGGGGAAGCTCAGTGAACCCAGGCTGTGGTCCAGCCCCACGCTTTGTCTAGGGCAGGTTCCGTTCCCTCAAGCAGGTG
ATGGGAATCCCGGACTGGAGCCTGAGGCTGAGCAGCCAGAGGCCACACCGCCAGGGCCAGGCCACACCACCAGGGCCGGCCACACCGCCAGGGC
AGGCCACACCGCCGGGGCCAGGCCACACCGCCGCGGCAGGCCACACCGCCGGGGCCTGCCTCATCGCCCCACAGTCTGGAATGCTCTGCACACG
CATCAGAAGGAGAGTCGCTGAGCATCACAAAGGAAAATAGAAAGCGCAGCCTCACTGCCCAAGGCCGGGGCAGAGATGACTTGCCTTGTGTTAC
TGCCTAATTAAACAGATTTGGGTAAACAGATTTGGGTAAACCAAGTAGTTTAAAGAAAAATGTTGGAATATTGCCATTTGAAAAGTATTATTAT
TTTGCAAGAGGAAAACAATTGTGAGTTCACAGGGACAAACCCAAACTCTTGCCAGAAACTAAAAATGTGATTTATTTACCTCTCGACCAGCGTT
AGCAAAAAGAGCAGGCCTGCAGGATCAGGACGCGGCTCTGGGCCCTCCTGCCTCAGCCCTCCGGCACTGTGAGAGGGGCACACACCCGGGGTGG
GTGTGCTTGTCCTGCGAAGTGCAGAACAGAGAAGAGGCTCCGAAGCACAGAGCGCCTCTGCCCTGGGCTTTGCACACATTCCTGACTTCTTAAG
ACACAGCTGTGCAGGACCATTGCATCTCCGTGAAGCTGTCCTCATTGGCCTCAGGCTGACCCGGGCCCCTGGCACTCGAGGGCCAGGGAAGGGG
TCATGTCTCCCTGAGACACACATGTGGCATCCTTTCCCCGCCCCCGTGCACACCAGTGGAGTGTCAGCTCAGCCCTGTTTGTGCTTTCTAAGAG
AAATCGAGGTTTAAAGGGGACTTTTTTTTGCCTAGCGTGGTGTGTGAGGCCCAGATGGCTGCAGCAGCTGTGCCGGGCTTGCCCGGCCTCCTCA
CCGGGAGCTTCCCTCTGGCTAGAGTTGCTGTTTCATCTGCCTGTGTCCCTGCGGCCCCACCCCCATGGAGGAAGCGAGGACCCTGTTCAGCCAT
GGCCTTCCTGTGAATTAGGCCTCCTTCAGGTGCAAGTGACAGAATCCACATTAAACTCGATTGAGCAGAAAAGGGAAGTTGCTTCCTCACCAAG
GTGAAGACCCCAGGGGAGGCTCTCAGGTCCCACTAGACTCAGGGGCTCAAGCAGGGCATGGAATCGGCCTCTGGAGTCCCTGTCTCCATGGCAG
CCCCGGGGTCCACATCTTCCCCAGGAGGGCACTCTTCAGGCCCCGGCTTCAGAGGAAGAGAACGCCATTCCCTGCGGCTGTGGGGCAGCAGCAG
GCACACCTCCCTGGCCTCGGGTGGGCTCTGCCCCTGCGTGGGCTATGGCGGGAGTGGGAACATTCCCGTGACCCCCCTGGAGTTTAGGCCAGGC
AGTGGAGGTCCTCGAGCAAAGGTCATCCCACAGGGAGTGCTCTGTCCAGTAGGAGCATGGCGCCCATGTCAGGGCACTGGCCTGGAAGGGAGCG
TCTTAGTGGAGGCTGTGGGTGCTGCCAGGCCACCTGAGAGGGGACATGCATTGTAGGAAAGGCCACAGCGCACTGTGACAGGTGTGGTATCCTG
GAAGGAATGAAAAACACTGCCCGGCCAGGCGCGGTGGCTCATGCCTATAATCCCAGCACTTTGGGAGGCCGAGGCAGGTGGATCACGAGGTCAG
GAGATCGAGACCAGTCTGACCAACATGGTGAAACCCCGTCTCTACTGAAAATACAACAATTAGCCAGGTGCGATGGTGGGCACCTGTAATCTCA
GCTACTCGGGAGGCTGAGGCAGGAGAACTGCTTGAACCAGGAAGGCAGAGGTTGCAGTAGCCGAGACTGTGCCATTGCACTCCAACCTGGGCAA
CAGAGCGAGACTCCGTCTCAAAAAAAAAAAAAAAAGAAAAAAGAAAACCACTGCCCTTTGGGTTGGTTTCCTGGGGCTGCCATCACAAATGACC
ACAAAGTGGGTGGCTTGAATGGCAGAAACATGTCCTCAGTTCTGGAGGCCAGAAGGCACAGTCGAGGGTCAGCAAGGCCAAGTGCCCTCTGGAA
CCGGCCACGGAGGTCCCTTCCCTTCCTCTTCCAGCTCGTGCTAGCCCCAGGTGCTCCTTGGCTCGTGGAGCCATCATTCCAGTCTCTGCCTCTG
TCGGCTCACCACATTCCCTTTGTCTGTCTTCTCCTCCTGTGAGGGCACCAGTCATCATTGGGGTCCCCTTAACTGACACCATCTTAACTAGTTA
CAACTGTAGAGATGTCACTTACCAAATTAGGTCTCATTCCCAGAGACCAGGGGACTTCAATGAATCGTTTTGGAGGACGCAGTTCAATTCACAA
CAGCATTGTTCTGAAATAATGATTAAGCTGTCTTGAGCCATTAGCAGGTGCCTCTCATTGATCTACCCTGAGAGGTGAAGGCCGTTACTATTGG
CTCCCGTAGGTGTACCGGGGACTGGAATCCAAACTCGGGCTGTGGGCAAGCGCCTTCTCACACGAGAGCTCACGTGCTCTGTGAACCCGTCCCC
GGGAAACTGGTCAAGTTAAAAACCCCCAGCCATTGACAGTCTCTGGAAATGGTCCTAAGGACACGCAGCAAGTGAAGACACATCTATTGAAAAA
GACCTGATTTTTTAAAAAACCATCTTTACTAGGAAAGGTGAATGTATTTGGTATTTGAACCAAAGCCACCCCCTCCTCCCCTCCCCCTGCCACT
CCACCAGCAGGTCTGCTGCAGCTGAGGACACAGGTGGGTGGGGGAGAGTGACTCTGGGAGGGCGGGATGTCGGTGTTTCTCATCCTGCCCCATC
TGCCTGTTGCTGGGGCTGCTAGGTCCTGGGGAGTCTGGTGGAGGGGTGGGGGCTCCCTTCTTCCCGGTCCCCACACTCATGGGTGGGAGGCTGG
GCCTTGGGTGTGGCTCACTGAGAAGACAGGGTGCAACTACCCTTGCCCCCGCTGACAGGGCAGTGATTCCAGGCCAGGGGAGGCCAGTGGGGTC
TCAGGATGCTGCCCCCGTCAGCACTCAACTCCTGGAGGTGGAGGCGTCACTCACACTGAGGCTCGGCACTGTCCCCATCCCCAGCTCCAAAGCA
TTGGCTCAGAGATTTTGCCTGGAGTGAGAAGCAGTAAGTAAAAGAGATAGCTTCTAATCTCCTCCCAAAAGAAACGACTTCATTTTCAGCAGAA
CATGGGAGAAGGTGAAAGCTTAGGGTCCCCTCGAAAACATGGAGGTTGTCATCAATGGCATTTGGGACATTCATGGATTTAATGAAGATTTAGC
CTAGATTTAGGAATGTGATAGCTGTGAGGGGCCTCCAGGGGTCAAAACAAATATCACACATGGACATCAGAAACTCTTGCTTCAAAGAAGGCAC
TATTTAGTTGGAGTAGTTTGTAGAGTAATTTATGCCTCCAGATATTGTTAAAAACAATAGAGCAATCCAATGGCAGCCGTGGAATTTAACAGCT
GGGTATGGTCAGGGAAAGAGAGGAAGAGATCTCTATCAATGTGGTCGGGGAAAGAGAGGAAGAGATCGCTCTCAAAATCCTGCATTCCAGGGTG
ACTCTGGGCACAGGTAAGACTCCTCTCCCTAAGGAGCAAGCCAGAGGCATAACTCTGCTGGTGGAGGGGGCAGTGTGTGGAATAGCTTCACTGC
AGTCATCTCAGAAGTCACAAGACAAGTAAATAAGCAAATAACAAGCCCCACACAGGGGGACCAGTATCCAGAGCTGCTATAATACATTGTCTAA
AATGCCCAGTTTCCAACAAAATAGGAGGCATGCAAAGAAACACAAGAGTATGACCCCCACATTTAAATTTTTTAAAAAGCAGACAACAGAAACT
GCCTGGGGAATGACCAGATATTGGATTTAATGGGAAAAGGCCAAAATATCCCTTATATGCTCAGAGAACTAAAGTAAATCTTGATTAAAGAAAG
CAGGACGACAATGTCACATCACGTAGAGACTGTTGATAGAATGTATAAAAAATAACCAGTGGAAATTCAGGAGTTGGAAGGCGCAGTCACTGAA
GTGAACCATTCACTATTGGGGCTCAACAATCGATTTGAACTGGCAGAAGAAAGAAAGTGAATTTGAAGATACATCAGTAGAGAGTATGCAAGCC
CCGAACACAGAGCAGATAGAATGAAGGAGACCAAGGGGAGCCTCGGAGAGCTGAGGGGCACCAGGGACGCCCTCCAGGGCACCAGCCCACACAG
GGGGTTCACTAGAAGGAAAGGAAAGAGGAAAAAAGGGATGCAAAAATATTAAAAGAAATAATGGTTGAAAATATCGCAGATTAGTTTAAAAAAA
TAACCTACAAAACCAGGAATCTCAATGAACTCCAACTGGGGCAAGCCTAGAGAGGCCCACAGACCAACACATTATGAGTAAAAATGCTGAAAGT
CAATAATAAGGAGAAAATCTCAAAAGCCAAAATAATAAAATAAGGTATCACTTACAAGGGAACCCCAATAAGGCTAACAAGTGACTTCTAAGCG
GCAACGATGTGAGCCTGAGGCAGTGGGATAACAGCCAAAGGAAAGGAGAGAGGAGGAGAGGGGAGGGGAAAGGAGGGGGAGGCAGAGGGAGGCA
GGGAGGGAGGAAGGAAACCTATCAATCCCAAATCCTATGCGCAGCAGAGCTTTCTTTCAAGAATGAAGGTGAAATAAAGACTTTTTCCGACAAA
CTGAGAGTTTGTTGCCAGCTGACCCACCTTACAAAAAATACTAAAGGAAGTTCTTCAGGCAGTGAGCAAGAAACTTCAGAGTAATTTTTGAAAT
CACATGAAAAAAACAGAGTGCCTCTTAAGGTAATTACGTAATTTCATAAGACAGTACGAATACATATTTTTCTCTTTTTATAACTGGTGTGAAA
AGTGATTGTATAAAATAATGTATATAAAATATAATGTTGGGCCTATAACTTATAGCAATGTAATATATGTGTAGTGTATTGCTAATAACAATAC
AAAGAAAGGGAATGAGAACAAAGCTATATTGGACTAAAGAAATAACTACAGATGGTAAAATAATGGTTATCACAATGTATTCTTAGGTTTGTAA
CATTACTGTTTACAATGTAGAATGTATAAAAGCGGGAAGGGAATGGAGCTAGCTACATAGGGATAGTGTTTTTGTGTGTCACTGGAGTCGAAAT
GTATATGGTACATACTAGAGCAATCACTAAAACAGTAACAAAAAAAGTTTAAAAACTCAGTAGAAAATAATTCACTTAATGCAAAGAAGTCAAT
AAAGAAATAAATGAGGGGAAGACATAAGAAAAATAGAAAATGAAAAGTAAAGTGGAATAGAAATGAAAAGTAAAGTTCAATAATAACATTAAAT
GTGAGTGGATTTAACAACCAATCAGAAAGCAAAGGCTGTCAGACTGAATTAAGAAAACATGACCCAACTTTATGCCGTCTAGAGGAGACATACT
TTCTACTCAAAGCTACACACATAGACTACAACGATGGGAAAAGACGACACACCAACAGCGACTTCAGGAAAGCTGGAGTGGCTGCTAATGTTAG
ACAAAATAGGCTTTTTAAAAAAGGTTTTATTAAAGAGGAATGTTTCGTAATGATAAAAGCACTAATCTGTGAGAAAGATACAACAATGATAAAC
ATACGTGCAGCTAATAAGAGAGCTCCAAAATCTATGAAGCAAAAACTCACAGAATGAGGGGAGAAGCAGTTCTACAACAGAGAATGGGGACTTC
GATACTCCACTTTCAATAATGGATACAACAACCAGGCAGATAACAAGGCAACAGAAGGCCTGAACAACAGTATAAACCAATTAGACCTACCAGA
TATCTATAGCTAGCACACTCCACCCAACGACAGCAGAATACACATTCTTCTCAAGCGCACAAGTAACATCCTCCAGGATGGGCCATGTTCTAGG
CCATCAACAAACTCAGGTGGTTTGAGGCCAGAGGCCTCTCTTTTAACCACCACACTAGGGCCTTCGGAGGAGGCAAGCAGAGAGTTGTCCAAAG
AGGCCCTCAGGACTGGGTGCAGTGGCTCATGACTGTAATCCCAGCACTTTAGAAGGCTGAGGCACAAGGATCTTTTGAGCTCAGGAGTTCAAGA
AATGAGCACTTATCCACTGGGCGCGGTGGCTCACGCCAGTAATCCAGCACTTTGGGAGGCTTAGGCGGGCGGATCAAGAGGTCAGAAGCTCAAG
ACCAGCCTGACCAACATGGTGAAACCCCGTCTCTACTAAAAGTACAAAAATTAGCCGGGCGTGGTGGCGCACACCTGTAATCCCAGCTACTTGG
GAGGCTGAGGCAGGAGAATCACTTGAACCCGGGAGGTGGAGGTTGCAGTGAGTGGAGATCACACCATTGCACCCCAGCCTGGGCAACAGAGCGA
GACTCCGTCTCAAAAAAAAAAAAAAAAAAAAGAAAGAAAGAAAAAGAAAAAAAAAGTGAGCATGTATTTTGCCAGAGTCTGGAGATTAGAATTA
AATTAGCAAACCAGAATTATAGAAAAAGCTATTTACTTTTAAGTAAACAGCTGAGATTTTTTTTTTTAAGTCAGTGTGAATGAAGCTCACAGCC
ATGGTTGGAGCTGAGAAAGAAGGATTTCCCTTTAGTTATGCACCTGTGTCAGCACCTTCTGACTTTCCTTCTAAAGTCTGGGGTGTTCCTGAGG
ATCCGTAAGTTTGGGGTTCAGGGTTTCTACAGCATGCTGTTACTTGTGAAACATCTCTTTAACCATGTCCCAGAGTTGCCCAGGAGTTTAAGAC
CAGCCTGAGCAACATAGCAAGACCTCATCTCAACAACAACAAAAATTAGAAATAAATTAGCCAGGTGTGGTGACATGTGCCTGTAGTCCCAGCT
ACTCAGAAGGCTGAGGCATGAGGATCACTTGGGCCCAGGAAGTTGGGGCTGCAGGGAGCCCTGTTCATGCCGCTGCACTCCAGCCTGCAAGACA
GAGCAGAAAAAAAGAATCAGGATCCTGGGCAGAGGGAGGAGAGGGGACCGGGGTCCAGCAAGCACTTGGGGATTGACTGAATGGCGTTGGGGAG
AGATGACTCCAAAGTCCTGGAGTGGCTGAGAATGACTGCGAGTGGCTTTTAGGTGGGGAGGTTCCTGCCTGGCCACTCCGGGAGGGGACGTGGG
GCTGAAGGGTATCAGGTGCCGTGCTGAGCAGTTTGGCCTTGATCCTAATGCCCTGGACACACGTCTAGGGTAGGAAAGTTGACTGATCCATTGG
TGATCTGAGTTTTTAGACATGGTGGTAGTCCATGAGGTGGGTGTTCATGCTAAGAGTTTAGACAGGGAAACCTATGAAGCCCTTAGCAACCCTC
CAGGGAAGGGGCGTGGTTAAAGAGATGTTTCATAAGTAACAGCATGGTATAGAAACTCTGAACCCCAAATGTATGGGTCCTCAGGAACACCCCA
GACTTTAGAAGGAAAGTCAGAAGGTGCTGACACGGGTGTATAACTAAAGGGAAATCCTTCTTTCTCAGCTCCAACCATGGCTGTGAGGTTCATT
CACACTCACTTAAAAAAAAAAATCTCAGTTTACTTAAAAGTAAATAGCTTTTTCTATAATTCTCGTTTGCTAATTTAATCCTAGTCTCCAGACC
CTGGCTAAATAAATGCCCATTTCTCCAGATGGTCTCAAGAGTCTCTGGACATCGTGGGGGCCCTTCCCTGTTGGTTGGAAGGTGCCTCAGGAAG
AAGGGGGTGGATTCTGAGTTGAGTCAAAACCTCAAAGACCCCTGATGGGAAAAGCTCTCAAGTGACCACCGCTGTGGGCCAGAATGCAAAACTG
CAGGAACAGAACATTCGCAGGAACAGAACACAGTCGTATTAAGTGATTTTCCCGAGCAGGAAGTGGCATCTGGCCTGCGGTTCAGTAGGGGGAG
GAAAAGGGTGGGCGCACCTGCCCCTGGCTGGCGCACCTGCCAGGTAGCCCCACGCGGCACCGCGTGTGCCGAGCGCCCCTGAGGATGGAAAGCC
CCACGCGGGGCAGGTGGCACCCACCCCTCCGAAGACGGGACGGGATGGAGCGTTGAGCTTCGGGGCAGCTCCGGCCCGGCCCGCGCTGGAGACG
CCCGCATCTGCCAGGATGGCGTCTCATAGCCCTGGTGCTCACACATGACGCCAGGAAGCCCCAGCAACAGTGACCGCCCAGGCTCTAGAAAATA
TTGGACGGGGTGGATGAACACCCAAGTGCGCTCCAGGAGAAGGGATTTGGCACCCCAAGGGGCTTTTAAAACGGTAAGCTTCTAGGGGTGTCTT
TGCCCCCAATAATCCATAGAAACAACAGTCATCTAAAAATAGTCTTGTTTTCTGTCCTAAGCTCCTTTTAACTTTGTTAGTCATCACCAATCCT
AAAATAAAACCCGTGTAACGTCTCCCCTAGTAGCGGCTATAAACAAACCTACGAGGAGGCAGGAGGAAGAGGAAAGGGGCGCAGGGCGCTCGGG
GAGCAGAGCCGGGGGCCCGCGGTGGCCGCAGAGGCCGGGCCGGGGCGCAGAGGCCGGGCGAGCTGGCCGCGCTCTGGGCCGCCGCCTCCGGAAC
TCCCTGCGCCTGGCGCGCGGCCACCGTGGTCCCGGCAACGGCATTAAACAGAGGGAAACAGACCCGGGATTCCGTCACCCGGGCGGGGGGATAA
GGACGGCTTTGAGAGCAGACAGGAAAAGGGAGCTTTTCTGCATGGGGTGAAAAAATTATTTATTGAAGGAGGAGGAGGCGGCAGCGGAGGAAGG
GGAGGGGCGGGAGGAGGAGGAAGAGCCGGCCGCCCCCGCCCCGGCCCCCGCTCCTCAGGAGCCAAGGGCAGCCTCGCCAGGTCGGTCCCGGGCT
CGAGGACCGCGGCTGGGGTCGAGGGGCTCAGTCTCCCACGTGACCGGCTGGGCGCGCCCCGCCAGACCCGGCCTCGGGATTCCCTCCTCCCGGC
GAGTCTCCGCCCGCCCCGTCCTGGAGGTGGGGAGAAGGAGGGCGGGGCGGGGGGGACGGAAACTCTCCCCGCCAAATCCTGGCCCCAGGCCTGG
GGACACTCGCGGCGGGAAGATTTGGAGGGGAGGGGAGGGGGAGGGGCGTGGGGGCGCGGCCTCGCTGGAGTCCCCCTCCCCCCCCGACCCCCGC
CCACCGGCCTGCGCGTCCTCCCGCGGCNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNTTATTGAAGGAGAGGGGCGGGCGCTCGGCGACTCGTCCCCGGGGCCCCGCG
CGGGCCCGGGCAGCAGGGGCGTGATGTCACGGCAGGGAGGGGGCGCGGGAGCCGCCGGGCCGGCGGGGAGGCGGGGGAGGTGTTCTCCAGCTTT
AAAAAGGCAGGAGGCAGAGCGCGGCCCTGCGTCAGAGCGAGACTCAGAGGCTCCGAACTCGCCGGCGGAGTCGCCGCGCCAGATCCCAGCAGCA
GGGCGCGGGCACCGGGGCGCGGGCAGGGCTCGGAGCCACCGCGCAGGTCCTAGGGCCGCGGCCGGGCCCCGCCACGCGCGCACACGCCCCTCGA
TGACTTTCCTCCGGGGCGCGCGGCGCTGAGCCCGGGGCGAGGGCTGTCTTCCCGGAGACCCGACCCCGGCAGCGCGGGGCGGCCGCTTCTCCTG
TGCCTCCGCCCGCCGCTCCACTCCCCGCCGCCGCCGCGCGGATGCCAAGCACCAGCTTTCCAGTCCCTTCCAAGTTTCCACTTGGCCCTGCGGC
TGCGGTCTTCGGGAGAGGAGAAACTTTGGGGCCCGCGCCGCGCGCCGGCGGCACCATGAAGTCAGCGGAGGAAGGTAAGCCCCGCGCGGCCTCC
GCCCCCGGACCCCTGCGCCCCCCACGGCCCGGGCCGCGCCCCCCGACCCCGCCCCCGTCGCCCGCACGGAGGGGTCCGGGCCAGAGAACGAGGT
CGGCCGGGTCTGTGCGCCCAGGGAGGGGCGCGGCGCGGACCGGGAACGGCGTCCCCCAGCTCCCAGCAGCCCGGTTCCGAGCGCGCCTTCTGCC
AATAGGTGTCTCCTCTGCCCCAGCGCTGAGGGCGCGAGGCGAGGCCGCCGCGGGTCCCGCCGAGGAAGCGGCTCGCAGGCCGGCGATTTGGGGA
CGGGCGAGGCTGGGCCGTCAGGACCACAGAGGGCACGTGGGAGGGAGGAAGGGAGGCTTCCTTGGGAGTGAAGGATGCCCCGTGTCTTCCTGAG
CGAGGCAGAGCCACCCTCTGGCGCCAAGATCGCGGTTTGGGGGTTCTGCGCCCCCCACCCCCACCCCGAGCTCGTGGCGGCGTCTCCCTCGCGC
CGCGCCCCTGAATTCCCCAGGGCCGAGCCCGTCACGCCGGCTCCTGGACCTGCAGGACCAAAAGATCCAAAGCGCGGCCGCAGCACAGCCCCGA
ATTTAAGCCATTTTGAAATCCTATAGGCAGGAGTTCCAGCCAGAGAAGATGTTTCAGGCAAGTTTGAAAAGGAACCGCCAAGTTTCGAGCTGCT
TCTTTATAGTGAAAAAAAGATGGCACAGTGTGGGTCCCTGAAACCCGGTGTGGGGGGCGCTGAGACCCCCCTGCGGCGCATCTCACATTCCAGT
CCCCCACCTTAGCCCGTCCCCGGCACACACCCCTGGCGATGCCGGGTGGGCTCCTGCAAGTGGCTTTGAAAATACCCTTGCAGAGGTGTGAGCA
TACGAGTTAGGGATTAGGGATTGCAATGCTAATGGAAATTGGAAGACGCTTTTCCAAATTCCTTACCAGCTCTGAAATATTTTATATAATTTAT
TTAAAGAGCGCCAGCACACCTGCCAACATATGTACTTCATAAGGGCAGACGTCCTTCCGAGCGCTGCCGCTTCCTGCGCGACCCTGATAACTGC
GGGGCTTGCTTGAGAACCGGGCTCCCCCAGCTCTGGAAAACTCGCTGGTCTTAGCAGAGCTGAGCACAGGGCCGTGAGCTCCACGGAGGGGGGT
CCGTGTGGTCGGTGCCCTGACGCTTCACGGGACGGGAGGTAGACACCACTTCATCCCAATGTTAGAAAATGTTGACATGTTTAAACCATGTCCC
GTGTCAGTGTTTTACACAATGTGCTCTTGCTAGTAGCAGAGTTTAAAAAATTTTTTTTTGTTGGAGTTTCGGTTTTAAATCCTGGGACACTTAC
GCCGCAGTGTTCATTATCACTCCCTTTAGAAAGGCAGGTCTTTCCAAGTTCAGATCTCTGTACCTGAAGAGCCGGTGGCCGACCTGTAGTAAGG
AGCCCCTTTCTTTTACGCAAGGAAAAAACACGACGGGGCAGTGGCTAGTTTTCGTGTTTCCTACTGAAGAAAATAAGCTCGAAAAGGTCCCTGC
TGCAAGGCAGTGCGCCGGGCAGCGACGCCCGACCGACATGGGTGTATTTTATGAGGGCTGCAGAACTGGGCCAGCCGGAAAGTGTGATCTAGTC
CTTGATACACACAGGCCGGCAGCATGGGGACACGCGTGGTTTCCACACCAGCCATCGCTGAGACCACCGGGAAGAACTCTGAGTCCTTCTGAGT
AGGCCCCACGAGGCCATTCCTGGCCCATCTGAGGACCTGGGCGCCTCCCCTGCGTCCCTCGGGTTTCGGGACCATCTGGGTGGAAGCTCATTAG
GCGTCGTGGTTCCTGAAGGCCCCGGGCTTGCTTCCTCATGGAGAACGCAGAGAAGTACAGGCCTGTAAGTAGCCACCCAGTCAGAAGTCGCTCC
AGACACAATGCCCCACGGGCTGGGGCCAAACAAAGCTCACCCTGTTCTCTGCCACTTAACTTCACAGCGTTGTTTCCCAGCTCAGATTAGAAAA
TGGCAGGGCGCTGGCACCCCGCTTCATCACACCCAGCGGGGCTTCTCTCTCCGCAGACATCCCCAGCCTCTGGGAGGATGCGTCCAAGCTAAAC
ATTTCCATGGAGACACCCAGTGTCTGTGGAACGGGCAGCTTTGAATCTGGACCAATAGCTCTGAACCATTTGCAGGAAGGAAGCCCAGCACTGG
GGCGGGGGAAGGGGGGGCTCCTAGGAGCAGCAGGGGAGCCGCCCCACCGACCCCTTCTCTGGGATGGAGACCAGTGCAGTCCTAGGACAGCAGC
AGTGACCCTCCATCCCTGCAGGGCCCGCGGCCTGGGGCCACCAGCAGGCGGGACAGTTTAGCAAAAAGTTTTAGTCTAAACAGATGATAAGGGC
TGTCATTTGTGTATAATTTTATGGTGGGGGTAAAAAGGTGGCTGGAGTTTTAAAAACAATGTACAGGCCGGGCGCCGCGGCTCACGCCTGTAAT
CCCAGCACTTTGGGAGGCCGAGGCAAGCGGATCACGAGGTCAAGAGATCGAGACCATCCTGGTCAACATGGTGAAACCCCGTCTCTACTAAAAC
TACAAAAATTAGCTGAGAGTGGTGGCGGGTGCCTGTAGTCCCAGCTACTCTGGAGGCTGAGGCAGGAGAATCGCTTGAACTCGGAAGGCGGAGG
TTGCAGTGAGCAGAGATTGCACCACTGCACTCCAGCCTGGGTGACAGAGCGAGACTCCGTCTCAAACAAACAAAAACCTGTACAATGGAAAGAG
AATCAAACGTTGTGGCTTCTCAACAGAAAACAGAACTTCCACTTGCCCAAGGTCGGCAAAAGCAGCAAAACTACAACAAAACAACGCTCCAACA
TCCATCCCAAAGTCAGAAGTATTATTAGAAACTAAATCAGGCCACACGGAACGATGGCTTATCACTCGAGAAAACCAGCCAGTGAAAGGGTCGC
GGGAGAAGCCCGGGGACGACCCTGGGACTGGAGGGTTTCTCGCCTCTGNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNAGCCTGGGGGCGGGGGGCACAGTGGATGCCAGTGAACATGGGTGGTCAGTCCCAGAGCTGGCGT
CTTGCAGCTGGGGCAGATCCTGGGGATGGTGTGTGTGTCCTGACCAGAGGCCAGGCTGGAGGCTGGAATTGGCGTCAGGATGCCCAGCTCCACC
TCCACCCCAGTCCCTGCCTGTGGCTCTGGGGAAAGAAAGACCTCCTTATTGATTTATTGATTTATTGATTTGACAGGGTCTTGCTGTGTCGCCC
AGGCTGGAGTGCAGGCTGGAGTGATCTCAGCTCACTGCAACCTCTGCCTCCTGGGCTCAAGCAATCCTGCTGCCTCAGCCTCCCAAGTAGCTGG
GACTACAGGCGTGCGCCACCATGCCTGGCTACTTGTATTTTTTGTAGAGATAGGGTTTCATCATGTTGCCCAGATTGGTCTTGAACTTCTGACC
TCAGATGATCCGCCCACCTCGGCCTCCCAAAGTGCTGGGATTATAGGCGTGAGCCACCGCGCCTGGCCACCTCCTTGATTTATTGACATTACTG
TTCTCTCTCCGGTGCAGGAGACTGCGTGGCTCCCGTCCTCTTGAGGGCATTTCTTTACGATGGGGAAGGCGGGACTGGGGCCTGGCTAGCACCA
TCCCCAAGGCGCTCAGCCTCAGCGGAGGGGCCCTTCCCATACGAAGACATCCTGAGCAGGTCTCACCTCCCACCCCTGAGAGCTTCTGGTCGCA
CCTGTGAGGTGTGGCTCCATCGCAGCTCTGTTTCTTTTCTCTTTGTTATGGCTCTGCCCCCTGAGTGTCTGGAATTTCCAGAAAGCAGCTGTGA
CTCCTGGGATTGAGGCCACGGTGTCTCCTCTTACCAGGCTGCCGTGCCGTGTTTCCCACCTACCTGGGGCGCTAGAACCATTTCTGTTACTAAA
TACAGTGGGTTTGTATTGGTATTTTGAGACCCTAGATTTTTTTGGCATTTATCCGTGTTGGTTTCTGAACCACGACTTAGCGGAAAGACAGCAC
TGTCCTTGAATAAAGTATCTTCCAGCCAGTGCAGTTGCACCAGGAGATGAATATAGAATTTCCATGTGCTTGTTTGTATTATTTAGAGGCCTGG
GAATACCCACCTCTAGGCATGTAATCATGAGGTTCCAAGATGTCATAAAAAGTCTTTTCAAATCAGAAATCCAGAGGCACGTCTTTCCTGTTAA
AATTACTGATTTCCCTAATAATGATTTTAAAAAATATTTTATTGAGTGTAATTAACACATAATGATTCTTAGGACTAGCTGTGTACCAGAATCT
TCTGTTTTGTTCTAAAAGATGGATTCGTAGGATATGAGCCAGTATCAGAATCTCCATGAGGGGGATTTCACAGTCTGTCTTTTCATCCAATCCT
TGAGGAGTTTTCTTTGTAGCTGGTGATTAGGGCACCTTGACAGTGATAAGCCTGTTAAAATCTGTTTACATATGTAGGTCCTTAGATCTAAAAC
ACATCCACCATCCATCATCATCCATCCATCATCAAACCATTATTCCTCCATTCCCTATGCATCCATGCATCATCATCTGTCCATTATTCCTCCA
TTCCCTATCCATTCATTCATCATCCATCCATCCATCATCCATCCATCCATCATCAAACCATTATTCCTCCATTCCCTATCCATCCATCCATCAT
CCATCCATCATCATCCATCCATTATTCCTCCATTCCCTATCCATTCATTCATCATCCATCCATCCATCATCATCCATCCATCATCAAACCATTA
TTCCTCCATTCCCTATCCATCCATTCATCCATCATCCATCATCCATCCATTATTCCTCCATTCCCTATCCATCCATCTATCATCCATCCATCAT
TATCCATCATCCATCCATTCCTCCATTCCCTATCCATTCATTCATCCATCCATCCATCCATCATCATTCATCCATCCATCCATCATCCATCCAT
TATTCCTCCATTCCTCGTCCATCCATCCATCCATCACCATCCATTTATCTATCCATCCATTCATCCACCATTTACCTATCATGCCTCCATCCCC
AATGCATCATTCATCCATCCATCCGTTTAGCTGTCCATCCATCCATCATCCATCCATTCATCCACCCACCCATAATCCATCTCTTTATCCCTCA
TTCCTCCCTTGTTTGTCCATCACCATCCATCTACCTACTCTCCATCTACCCATCATCCCTTCACCCCCCGTCCATCATGATCCATCCATCAGTT
TCTTTACCTGTCTATCCATCATCCATTGTCCACCCACCCACCAACCATCCATCCATTAAACAAATTGAACACATTTTATGTGCCAGGCACAGTC
TTTGGCTCTGAAAACAGCGGTGAGCAGAATAGACTAAGGCGTCCAGATGTTCTGTCTTCATGGAACTTAGGTTTCTGTTTGTCAAAGAGATGAC
AGTGTGGGATGAATGAGTAAAAACAGGCTGTTAGATTGTGATACGTATTTAGGAGAAAAAATAAACTAGAGAAGGATGGCCCACGTGTTGAGTT
TGGGGTTTAAATGAAGATGGGGTGGTTGAGGAAGGTGGTTTTTGAATGAAGAGTGGAACCCGTGAGGACCCAGTCGCCTCTCTCTGGGAAGGAC
GTTCGGATGAAGATGGGGTGGTTGGGGAAGGTGGTTTTTGAATGAAGAGCGGAACCCGTGAGGACCCGGCCGCCTCTCCCTGGGAAGGACGTTC
GGGGGCTTGTGCTGCTGTTAGGGGAGGAGGGGAGGTGGGCAGTGAGGGGCTCACGGGAGCCTTGTTGGCCAGGTGGGACTGGGGCTGTCACTCC
AAGTCGCCCGGAGCTGTCCGGCAGCGTGGTCTCAGGGACGTTTGCTGAGGCCCGCTCCTTGGGGTCCGTTGGTCGAGGCCGGGGGTTGCTGGCC
GGCCCTGAGTTCATGGGTTTCTGCTTTGTGATGCCCAGCCCCTCATGCTCCCATCTGCTTCTTTTTCTCTCTAGAACACTATGGCTATGCATCC
TCCAACGTCAGCCCCGCCCTGCCGCTCCCCACGGCGCACTCCACCCTGCCGGCCCCGTGCCACAACCTTCAGACCTCCACACCGGGCATCATCC
CGCCGGCGGATCACCCCTCGGGGTACGGAGCAGCTTTGGACGGTGGGCCCGCGGGCTACTTCCTCTCCTCCGGCCACACCAGGCCTGATGGGGC
CCCTGCCCTGGAGAGTCCTCGCATCGAGATAACCTCGTGCTTGGGCCTGTACCACAACAATAACCAGTTTTTCCACGATGTGGAGGTGGAAGAC
GTCCTCCCTAGCTCCAAACGGTCCCCCTCCACGGCCACGCTGAGTCTGCCCAGCCTGGAGGCCTACAGAGACCCCTCGTGCCTGAGCCCGGCCA
GCAGCCTGTCCTCCCGGAGCTGCAACTCAGAGGCCTCCTCCTACGAGTCCAACTACTCGTACCCGTACGCGTCCCCCCAGACGTCGCCATGGCA
GTCTCCCTGCGTGTCTCCCAAGACCACGGACCCCGAGGAGGGCTTTCCCCGCGGGCTGGGGGCCTGCACACTGCTGGGTTCCCCGCGGCACTCC
CCCTCCACCTCGCCCCGCGCCAGCGTCACTGAGGAGAGCTGGCTGGGTGCCCGCTCCTCCAGACCCGCGTCCCCGTGCAACAAGAGGAAGTACA
GCCTCAACGGCCGGCAGCCGCCCTACTCACCCCACCACTCGCCCACGCCGTCCCCGCACGGCTCCCCGCGGGTCAGCGTGACCGACGACTCGTG
GTTGGGCAACACCACCCAGTACACCAGCTCGGCCATCGTGGCCGCCATCAACGCGCTGACCACCGACAGCAGCCTGGACCTGGGAGATGGCGTC
CCTGTCAAGTCCCGCAAGACCACCCTGGAGCAGCCGCCCTCAGTGGCGCTCAAGGTGGAGCCCGTCGGGGAGGACCTGGGCAGCCCCCCGCCCC
CGGCCGACTTCGCGCCCGAAGACTACTCCTCTTTCCAGCACATCAGGAAGGGCGGCTTCTGCGACCAGTACCTGGCGGTGCCGCAGCACCCCTA
CCAGTGGGCGAAGCCCAAGCCCCTGTCCCCTACGTCCTACATGAGGTGAGCCGGCAGCGCGGGGCGGGACGGGGAGGCGAGGGGAGGCGCGGGG
CGGGGCGGAACGCGGGGAGCGGGACGGGGGGCGGCGCGGGGCGGCCGTGTCTGGGGACGAGAGTCAGGACCTGGGTGGGCAGGTGGGCTCCTCG
TGGAGCTGGGAGGCAGAGTCTGTCCCCATGTCTGCCTCCTCATGCAGACTTTTCCTTGTGCGCCTCGCCTCTGCCTCCGTCTGCGCGTTTCCCA
TCTGAAGTCTCCAGATCCTCTCGGGCCCTGCTGATGGCCTCGTGTTCGCTCAGTCTCTGCAAACACCCATTTCAAAGAGGGTGGCCCTGCTGGC
GGTCAGGGCTTCCACGTGGGAATTCCGGGAGACGCCATCCGCCCCTAAGGAGGGCTCACACACGCGCAGTTTCTTCCTCGGGGAATTGCACGTG
CCTGGCCCCCCTCGGTGGTTGCCTGCTGCTCCCGCTTCCTCCCCTGCAGTAATTGCCTTCCCTGCCTCGGGTGCAGATGGGACCGCAGCTCAAC
AGTGCCCCAGAGCACCCAGTCCGCCACGCTTTGGAACAGCACGGCCTCCTGCTCCCCAGAGACGCCGGCTGTGGCATCAGGCTGGGGGAGCTCA
CGCGCCTTCCTTCTTTGGGTCCCAGGTTGTTGGTTTTTCTTTTTAACCGTGTGAGGGGCTGCTGGAATTCCGCTGGGTCCACTTAACCAGCTTG
GTGTTTTTATCTCTAACGAAACAGCTTTCCCAGCTGGTGGGCTGTGAGTGGCCCCTGAGAGTGCTTTCTGCTCTGGGATTTTTATCTGAGGCAT
GAAAACGTCCGTGCAGGAGGTCGAGGGCAGTGCCCCATGGCCCGTGACGTCAGGGCTGCCGCTGTGTCCTCAGGAAGAGGCGTTTGCCACGCGC
AGCCCGGGGTATTCCAGGCTGGGTGTTTTCCTGTTTTTGTTTTTTTTTTTTTTTCGTCCCTCCCCACCCCCCACTCCTTTTTTATTAGCGAGCA
CTCAGGGGGCGAGACCCCGCAGAGCGGAGGGCAGGTGCAGCCCCCATGCCTGCAGGGTGAGGAAGGCAGGCAGTGAGGGTGCTCTGGGAGGACC
CGTCTCTGCTCTGCAGTCTCAGGCCATGCTTCAGCCATGGCTGGAGCACGCATGGACAGAATTAGAGCTACAAGCAGTAACAAGCCTCCAGGCT
GGGAACGAACCCACCCAAACCTCTCTGAAGGCTTGTGGCAAAGAAACAGTGTTAGAGCTGACAGTTCTTTGAGCAGCCCCATGGCTGATTCTGC
CTTTCTACTGGCGTGATACTTAGAATCGTCGTCAGCTGAGGTTTCGAGAGAAAGCAAGGATTACCCCTGATTTTTCTTGACTTGCAGTGGGCGC
ATTTACCTTTCTATCCCAACTTCTGTTTTTAATACATTAAGGTTTAAAAGGCTGGCGTATTCTAGGTGATCACAACGAGCCTGTGTCAGGACCC
GGATTGTATTTACAATAAAAATCCCATTCAGTTGGAGGCATTGGGAAAATTTAAGAACTGTTTTTGCCTGCTGTACTATCACTGTTATTGTTTA
AGTAAAAGTACAAGCGTAGTGGCACTGACTCTCCAGCCCACAGTCATGTACAACGCGCTTCATGGTCTCCAGGCTTACGGACGGTGACAAAGTC
GTCCATCACCGCGGAACTTCACTGAACTGGCGAGATAAGGGAAGAGTGATGTCATCAAGGTCCCAAGACTGGGACTTGGCATGAGGAGTGTCGT
AATGCAGTTGTGAACCTTTGGGTGAACCCCACGTATTTGTTTCCTGGGGCTGCCCTGACAAGGTCCCGCAGACGGGTGGCCTTCATAGCAGGAA
GGTTTTCTCGCGGGGTCCTGAGACTGGAAGCCGAGATCAGGGGCTGTTCTTTTGGAGGCTGTGAGGGAGCATCTGCCTGACCTGTCTCCCTGGC
TTGGAGGCGCCGTCTTCTCCCCATGACTCCTCATCTCTCCATCCACCGTGCATGTCTGTGTCCAGATGTCCCCTTTTGTGAGGACACTGGTCCT
GCTGGGTTGGGGCCACCCTGGTGGCCTCATTTGAACTTGATCGCTCTGTAAAGACCCTCTCTCCAGACACAGCCCCACGCTGGGCAGGACTCCA
TTATGTGAGTTTGCGGGGAGCACCGTTCGGCTGCCTCAGCCCCGGTCTTACTGTCGCAGTAAAAATGGCAGGCAGCCTTTGAAAGCGCAAGCTC
GTCCTGGTTCCTTCTTCGTCTCCTGGGCGGCAAGGTGTCCGCAGGGATGTCGTGTTCTGGCAGGCACAGCCCCTCCTGTCCACCTTGGACCTGT
GTTTGGTGTGGAGGTTGGAAAGCATGGGTGGCTGTTTATTTTTTCCTCTGCTTGGGGGTTTCAGGAAGAATTCCGTGCCCACGTGACCCTGTTA
GCTTTATCTCCTGGCTGCAGAGCGCCCAGTTAGGTGTGATGCCTGTAACATTTCTGAGTGTTCACACAAACAAAGCTCAGTAAATATGTCCCAA
ACTCGCTCCTGGGGTTTGCAGTTAACCTTTTCCCACTGTTCTGGTTCCACATCAAAATATGTTTCCTGGTTCCTTGTGCCCTGTTTCCAAGTGA
CCTCGTCAGCGGGTGCCCTGGGGCAGCTGGAAGCCACGGCAGAGATGAAATGTTAGTTTTTTACCAGCGAGGTGGAGAGGCCAGACTTCCTTCC
CCAGGAATGCCTCTCCGTCGGTCACATGCAGCAGCTCTGGCAGGCGTGGAAAATGTCACCAGGATCTCAGGCTGCACAACTAGAAAGCTGTATT
TTTAATGATCTCCAAAGTTTGGGATGGAGAATGCACGACATGCCCATCTTCTCCCCACCGTGGTACAGAACTGCTGCTCCATTAATCCTGTCCC
TATGCCAGAAGGAGCTTTCTGGTGGAAAGTGTGCTGTGTGCATCCTTCCCAGGGACCGTCTAGGATTGTTGTCCTTTGAGGCCTTTTGTTGCTT
GTTCAGCACCTGTGGCCCCCAGTGCTATGCAGAGCCTCTGAGAGGCCCACAATGGTCAAAGAACGATACTGAATTCTTGCTGCAATTGAAGCCA
TGATAATGTTTGTGAAGTTAGTAAGAGTAGGTGATAGAACCTTGATAGTTTTAGAGTTTGGAGCCATTTTAGAGCTCAGCGGAATGATGTGTCT
ATTGTCAAAGCTTGGGGGGACTGGCACAGGAATCACAGGCCACCTGCTATACCCCCTAAAAATTAGAATCTTAAGCTGTGAATTCTGGCATCAG
AAAGGAGTGGTTTTCTTCCCGCCTTGCTTCTTTCCAGGGGTAATGACGGTGATCATAGCAAGTGGCCTCCCAGGGCACCTTTCCGTGTGCTGTT
GAGGGTGCAACAGGAGGCGTTGCCGGGCTGCGTCTGGAGGAGGCCAGGGCGAGAGTGCACGTTGAAGGCTGTGCGGGTTATTTATAAATAGTGG
TTCTCAAATGGTAGCTTTTAAAGATTTCATATTGACCGACAGGAACTAAAGTCTACGTGATTGCTTCCTCATTCCTTCATCGTTCGGCCTCCTC
CACCTGGGCACCTGCCACAGGCCATGTGCGTCCATGTCCAGGTCTGTCTGTGCCGACATCTGCTCTTTGGGCATCAGCCTCTGGAAAGTGTCCC
TGTCAGAGTCTCCACGTGTCTCACTCTCCGCACCCAGACAGTCTCTGGAGTTGCCTTTGTAGAGGCAAGGTTGTAGGGGAATTGGAAAGGCGGA
GGTATTTTGACTGAATTTATTGGTAACTTTAACAGCACAATTCTTGAATTCAATTTCTTTTTATTTTATTTATTTATTTTTTTTGAGATGGAGT
CTCGCTCTGTCACCCAGACTGGAGTGCAGTGGCACAATCTCGGCTCACTGCAAGCTTTGCCTCCTGGGTTCACGCCATTCTCCTGACTCAGCCT
CCCAAGTAGCTGGGCCTACAGGCGCCCACCACCGCGCCCGGCTATTTTTTTGTATTTTTAGTAGAGATGGGGTTTCACCATGTTAGCCAGGATG
GTCTCGATCTCCTGACCTCGTGATCCACCCGCCTCGGCCTCCCAAAGTGCTGGGATTACAGGCATGAGCCACCGCGCCCAGCCCTTGAATTCAA
TTTCTACACATGAAACATTGCCTCTTAGAACTGAATGTTTAAAAAGAATTTCTAAGGAGTGAGGTAAAGAAAAAGAATTCCTAAGGGGCGAGAT
AGGCTAATAAACAATGTTTACTTAGAGTCCGCTTGGTTTCAATGATCAGAAATAGCCGCTAGGTTGGTGCAATAACGTATATTCAAATCTCTTG
GCCAGGCGCAGTGGCTCACGCCTGTAATCCCAGCACTTTGGGAGGCCGAGGCAGGTGGATCAGCTGAGGTCAGGAGTTCAAGACCAGCCTGGCC
AAATTGGCGAAACCCCATCTCTATTAAAAGTACACAAATTAGCTGGGCATGGTGGCGGGCGCCTGTAATCCCAGCTACTCAGAAGGCTGAGGCA
GGCGAATCATTTGAACCCAGGAGGCGGAGGTTGCAGTGAGCTGAGGTCGTGCCACTGCACTCCAGCCTGGGCGAGAGGAGCGAGACTCCGTCTC
CAAACAAACAAAAAAACCCTGTTTATTTCAGACATTAAGTTTTGGGCTGCAATATTGAAGATTTTAGCATATCGGTCACTTTTCTGCTATGCAT
AGTATTAGTTTCAAAAGGTCTGAGAAAACGTTCACTCTCTTGTGTTTCATAAGTTATCTGAAAGTTTCACAGTTTATCACAATTTGGAATAAGA
TCAGCTAACCCCCAGCACAGTAACAGGTGGACTCTGTCTTCTCTTTCGCAGACACGAGTCCTCTGCCAGGCTCAGTAGCGATTCCTCCTGCGTT
TGAATGCCCTTTGCTCTTGTGGCTCTGGTACCTGAGGGGTTCGGGCCCCGGCAGTGTTGGGGGAACAGTGCAGGCTTTTGACAGCTCCCAGGTC
CTGTAGTGGGGCCATCTCTGGGCTCTGGAGCCAGACTGTCCGCGGGCGCTGGTGGGCCCTTTGGGGTGATGGTGAACATAGTCTCTAAGGCGTT
GTACCGAACCCCAGCATAGCGAGACGTGTAAGGTGGTAGCCGTGGGGTCTTCCCCGAGCTTCCCGCTGCCATCACCTCCTGGGAAGACGGATAG
CAAAGAAGCCGAGGGTGTCGAGTTGCCCCATGGTTTGTAAAACACTGGCATGGGCTGGAACGGCTCAGGACTGACTGCCACTGCGTCTCCCTGT
AAGCCCCTCCCTTTCTGGGGAGGGAAGCCCCCCAACAACCCACAGCCAGCCTCGAAGCCACGTCGCCCTGGCTCTGGTTCCCGCTAACCCTTGC
TGGGGAGTCCCCCGCACAACCTGTCCTGCTGTTTCCTGCTGTTTCTGGAGCTAGACAAGCCTCAGCTGGCCTCCTGGCTCCCGGCATGGGTGTC
AGGCCCTGGGGAGTCTCCAGTCCCTCCTGTGGGTACTGGGCTGGGCTGGCTCTGAAGGGTCTTGAAGAACGTGGCTGTGTGCCTCCCTGGCATT
CGCGGTGGTGGCTCTGGAGCTTGCGTTTGGCTGAGCAGGCCAGCAGGTGACTCGCGCAAGCCAGACGGTCCCCAGGAAGCCCTCAGCTGTGGTG
GGAGATGGGAGATGGGCTATGGTGGGAGATGGGCTGTGGCGGGAGATGGGCTGTGGCGGGAGATGGGCTGTGGCGGGAGATGGGCTGTGGTGGG
AGATGGGAGATGGGCTGTGGTGGGAGATGGGAGATGGGCTGTGGTGGGAGATGGGCTGTGGTGGGAGATGGGAGATGGGCTGTGGTGGGAGATG
GGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNATGGGCTGTGGTGGGAGATGGGAGATGGGCTATACCAGGAGATGGGCTGTGGCGGGAGATGGGCTATGGCTGGGCCTGTCCTCTGTGGTGC
TGTGGATGCGTCCTATTCAGGAGCTGCCTACCAAGTTTCATTGCGATGTTGGAATCTGTCCTTGGTCCCTGCCACCATGGGGTGGGTGGAGCCC
CCAGCACCCTGCCTTCTTCCTCCCCTTCCTGTGGGTCTGCAGGGCTCGGGGTGTTGATGGAGACTGTGGGGGTCGGGGGAGCCAGCAGTTTCGT
TGTGTGATGGCAGGAAGAATCCGCGTGCTGCCCCACCATGCCTTGCGACGCTCTGTGGTAGGGAGCTGTCGGGTCACAGTCAGGCGCTTAACGG
CCACCCCACTGTGGACGCAGCTGAGGCCATTTGTCCAGGGTGGCCTAGTCACAACCAACAGAACTGGACTCTGCCATGATGAGCAGACCCCGCA
ACCTACACTCCAGACTCCACCGGTGCTGGGACCCCGACTCCCAGGAGACCTGGCGAGCTCAGCCCTCTTGTCACAAGGCCTGTACGGGGTGGGG
GCACGTGTCACGGGGACCCTCGGCCCAGACTGGCCTTCTGCTCTCGTCACCAAGATGTGGAGCCTTCTGGGGTCAGAGCCCAGTCGAGGGGCTT
TACGCAGCCTGTGGTGTCCTTGGAGCCACAGCTCCCTGGAGAAGGCACTGCGTCTGCCAGCCTCCGCTGGTGAGGGGCTCAGAGTTGAGAAGAT
CATGCCTCCTGATTGCACGAGCACCCTGGTCTTGGCTCATTTGCTCCTTCTTGAGCCGCTTCGGTAGCATTTGGCAGCCCACGGTTTTCTGCCT
GCGTTGTTTAGTTTGTTTCTGCTGTTATGGAGTTCACAGCTGACACTTGGAGGGCGGGGTTTGCAACACCAGTTTCTGATGGGAAGGACTGGTG
TGAGGGTGTGAGGAGGCTGCGGAGGCCGAGGGCCTGTGCGTTCTCCCAGGCGTGTGTGGAAGGGCTGGGGCATCTTCTCCCTGGTCCTTCTCAA
TGTTACTCTGTCCATGTCTGTTGTTAGCCATGAGGAGGAGAAGGCACTCTGTTCCTGGAAAGGAGAAAACAACGTGGCTTTTTGCTCAGATATT
TTTTTTTGAGCCAGGGTCTTGCTCTGTTGTCCAGGCTGGAGTGCAGGGTGTCATCACAGCTTACTGCAGCCTCGACCTCCTGGGCTCAGGTGAC
CCTCCCACCTCAGCCTGCCGAGTAGCTGGGACCACAGACGTGCGGCACCACACCTGGCTAATTGTTTAAGTTTTGTGGAGACAGAGTCTTGCCG
TGTTGTCCAGGGCTGATCTGGAACTCCTGGCCTGAAGCAGTCCTCCCGACCTGTCCTCTCAAAGTGCTGGCATTGCAGGTGTGAGCCAGAGTTT
TACAGTCAGATCATGGAGATCTGAAATGTTGCCCCATCTCACCGAAGACCGAGTGCCCGGGAGCCCCCCTAGGAGGGTCAGAACCTGCCCGGGA
GCCCCCTAGGAGGGTCAGAACCTGCCCGGGAGCCCCCCTAGGAGGGCCGGCACCTGCCTGCCCGGGAGCCCCCCTAGGAGGGCCGGCACCTGCC
TGCCCGGGAGCCCCCCTAGGAGGGCCGGCACCTGCCTGCCCGGGAGCCCCCCTAGGAGGGTCAGAACCTGCCTGGTTAGCTCAGATGCCCCCGT
GGAGACAGCGTGAGGGCGGCAGGGAGCTGGAAACCTTCCCAGGTTCCCAATTTTGAGATTTCCTAAGCTGCCTGTGGTCCAGCTGGGAGGAAGT
GTCTGGTCCTGATGTGCGTCGGTGACCACAGACAAAACAGGATTTGCTCCTGCGCCAACGTTGGCTTTTGAGCAGCGGGATAGGTGGGCCTGGT
GGTAGGTGCTTCGGCAGCCGGGCAGCCGCAGGGACTTCCTCCCTCGGGATTAAACCTGAGTCGGGAGTTGGAGGGCAACAGTTATTCACATTCA
AAACCAGATCTTAAGTGCAAATTAACGTTTTTTGGATTCTTGAGCAAAATGTTTTCTACATTATGATGGGATTTTCTTTTTTATAAGTGCTTCT
AGAACATCTCTGGAAGCTGGGTTAGACGCACAGGGCTTAGTCGGCTTTGGGGAGAGACAGTAGTTGTAAAGGCCACACCGGACGCCGTGTGCAC
CTCCCAAGGTTTCCCATGTGAGACCATGTGGCCTGCATGCTGCGTTCCATCAGCACTGGGCTGTGCGACAGATGTGTTTCCAGGCGAGGTTGCT
GCAGAGGGGAAGAGGGGGACGTGTTTCCAGGCGACGTCGCTGCAGAGAGGAAGAGGGGGACGCGTTTCCAGGTGACGTCGCTGCAGAGAGGAAG
AGGGGGACACGTTTCCAGGCGACGTCGCTGCAGAGAGGAAGAGGGGGACGCGTTTCCAGGCGACGTCGCTGCAGAGAGGAAGAGGGGGACACGT
TTCCAGGTGACGTCGCTGCAGAGAGGAAGGAGGGTCGCGTTTCCAGGCGAGGTCGCTGCAGAGGGGAAGAGGGGGACGCCTTTCCAGGTGACTT
CGCTGCAGAGAGGAAGAGGGGGACGCCTTTCCAGGTGACTTCGCTGCAGAGAGGAAGAGGCAGACGCATTTCCAGGCGAGGTCACTGCAAAGAG
GAAGAGGGGGATGCGTTTCCAGGTGACATCGCTACAGACAGGAAGAGGGGGATGCGTTTCCGGGAGACGTCGCTGCAGAGGGGAAGAGGTGGAT
GCGTTTCCAGGCAAGGTCGCTGCAGAGAGGAAGAGGGGGTCGCATTTCCAGGCAGCATGGCTGCAGAGAGGAAGAGGAGGACGTGTTTCCAGGC
GACGTTGCTGCAGAGAGGAAGAGGAGGACGCGTTTCCAGGCGATGTCGTTGCAGAGGGCAAGAGGGGAACGCGTTTCCAGGTGACGTCGCTGCA
GAGGGGAAGAGGCGGATGCGTTTCCAGGTGACATTGCTGCAGAGAGGAAGAGGAAGATGCTTTTCCAGGCGACGTTGCTGCAGAGAGGAAGAGG
GTGGGACGCATTTCCAGGAGACGTCGCTGAAGAGGGGAAGAGGGAGGCCCCTCCAGCAGGGCCACCTAGATGTTTGCTTTGCCCCGCGTGGAGT
CATATTCCAGGAACCCCCGAACCCACCTGCAGCTTGATTCTGCTCCTCAGGGCCAGAGGTTCTAGTTTATGGGAGGAGTGTTTTCTCACTGGAA
ACATGGAGCCCACACACACTTCTACGCTGGAAGCCGTGATGTGTGTCAGGAACGTGTTCTGATGTTGGTGTGCAAAATCAGTATTGTGCCTTTG
TCCCCTCTGACCCCCATCACGGCAGTTCTGAGGCTTGTGGGGCACCCCAGCTCCTCTCCCTCTGCACAGGCCACTGTTCCCAGAACTCGAGGGT
TCCCTGACACCAAGATGACATGTGGCACAGACCTGGAATCACGAGGAAATGCCCGTCCTCCTCCCACGGGCAGACAGGTCGAGTGTGTTTCCAG
AGACCGGGCCGTCCGTGGGGCAGTAGCGCCTGGATGACGTCTGCTTTCGCCTGGAGCCCTGGCTGCAAGGGGCGGTGCCGGGCAAGGGCCACCC
CAGGGGTCTGGGAGTGCAGTGTCGGGCCTGTGGAGGTGGAGAGCAGGGATCAGCCTCTTTTTGTGGATGAGGAAATGGAGACACGTGGAAACTT
CTGGTAAGAAGCTGTCAAGCCTCCAGGTTTCGGTTCTGGACAGGTGCGGGGCAGGTATTGGAGCACTTTGGGCTCTGAGGTCCACGTGGCCCAG
CGGCCTCCAGGAGTCAGCTGCAAGGCGGAGTCCCTGTCCACAAGCTGCGTTGCAGGCACAGAGACCAGGAGAGGGCTGTGAAGCAACTCTGTGT
TTACTGGGCTGAAGCAGCAGCTCTTGGCAGTGCCGCCACCATGGGCTGTGTCCTGTCCCCGTGGCGACATCACAGCTGAGCGCGAAGCTGGAAG
CTTGGGCACGTCTTGGTGCTGGGTCTTCTCCGACCCCGAGGGCCAAGGGTGCTCTGTAGCTCCAGGCGTCAGAGTCTGAGCATCCTTTGTTTTG
GCAACATCCAGGACAGGAGTCTTTGGAGAGTAACACATCCTTATTTTGAAAGAGCCCTTTAGGTGATTTGAGAAACCGCTGTATTTGTTGTAGC
TCCAAGTATTACCCGTGAATCACCTAGGCTGCTCTTCAGTGAACATCCAGAATGCCGTCAGTTCTTGTGAAACCCCCAAGTTTCCCGTAGGTCC
AAGCTGAGAAGATTGAGTGAACTCTGAGAGCTGAGCCGGGCCGTCTTGGCACCACTAGAAACGGAATCAGTTCCATGACTGATCTGTGCCCCAC
CCCACATTAACTTGTGCAATATGACATTTCTGGTTTTGGTTTTTGTTTTATAAGCCATCCATGGCAAGTGCAGTCGGAGGCCCCCCACTGCCAA
AACTTCCTGTGAGTAATAATGACAGATGGGCCCTGACCCTGGAGACGTCTGAGAAGGTGTTTGGGGTCCGAGAAGAGTCCGCGTGTGCAGTGAC
TGGAACCTGTCCCTGGTGTGAACTTGGGGATGAGTGGGTGACGGGGGTGGTGGGGGCCTGGGGCACTTCCCCGCCCTCCGGACCACACTGTTGC
GGCCTGCTGCCTGGGGCGCTGGGATCCCACAGCGAGGCCCTGTTCCCACTGCCCACAGGGACTGAGATCCCACAGGCAGGCCCCGTTCCCACTG
AGCTTGGGTTCCCAACGGGCTCCTTTTGCTTCCCAAGCACCCTCTCCCACCCTTGAGCTGCAAGGAATGTTTCAGAGCCATGGAAGGTGCTTTC
TCCCATCTGTAGTTTTTCTGGGAGGGAGAAAGGCTGACATCGGGGGCTTTGTAGAAAGCGTGCTCTGGCCCACACGTTAACATTTGGGAGGAGG
CCTACAGGGAGCTGTCGGCTCTGGGCGAGGGCTGGGGGCTGCCTCGGGGGCGGGGGTTCAGGGTCTTGAGCTCAGCTTCTCACCCATCCGGGGC
AGGGCAGGGCAGATAGAGGGGTGGGGACAGTGTTGGGGGCTGCTGGGCCTGGGATGGCAGGGCCTGTGTCCAAAGCTGTTGTGAGGCTCACGAG
ATACAGGGGTGGACCTGGGAGGGTTGGGGCGCGTACAGAGGGGATGACTTCGATTTTTCCAAAGAACAGGGAAGGGTCTGTGTCCTGGTCGTAC
TCCAATCCCGTGTGGTGGAGCCACGGGACGCCATCAGTTTTTATGAAATCCCCAAATGTGTCCTTTAAAACAAAACCTCTGCACTCCGGCCTCA
CAGCCCAGATCCCGCGCGCTCCCGGTCTGTGGCTTTGACTTTGGTTTGAGTTCCTGGGCTGGGCTTACCTGGCTGGCAGGGCCGCAGCTGTGGC
ACTCCCTGTCTGGGGACGGAGGCTGCCCATGCACCCGGCTGTCCACCTTCTTCCCAAAGGCGGCGTCCTGGGCTCCCTCCCATGCGGACCCGGG
ATCCACTTGCCCAGCCTTGGGGAGGACAGATGGGGACAGCAGATCCTGCCCCAGGGTCATTGCCCGTGGCCACATCGGTCCTGGGAACTGTGGG
TAGGCCATGGGCCGCTGGTGCCTGCACCACTGTCCTGGTGGAAGGGGCTGGACCTGGGACTGCCTGGGACAAGAGGCCAAGGCACAGATGGCTG
CAGGGGCTGCCCCTGCATCCTGAGCCTCACCCGGCGGGCAGGAGGGCCGGGCTGGGTGTCCCCATCTTTCTGTTCCCACCCTGGCCCCAGTCAC
CTGCTTCTCATCCACCGATTCCATCTTTCCTGCTGGCTTGTGAGGCCCGTGCCCTCCTCTCGGCCGCCTTCCCTCCCATGGGCACAGAGCTTGG
GTCCCACGTGGAGAGGAGGCCCTGCCCGCAGCCTCAGAGTCAGGCGGCCTTGGCTTCAGGGAGGGTTGGACGGGAGCCAGCCCATTGGACAGTA
GCCCGGGGCCTCTGGGTGACCCTCCAGGGTGGGACAGCAGTGGTCCCAGGCCCCCCTCTGACTCCTGAAATCCTAAGGTTGCCCGGGCCGCCGA
GCCCTCCCTCCACTCCCCGGCCTGCTGGGCAGGATTTTGTGAAAGGCCTTGTCTTGGGTCGTGGAAGAAAACCACGTGGTCTGTGGGGTCATCC
CTGGGGCAGGCTGTGGGAGGGTGAAGGCTGCGTCCAGCGGCCAGGCCCAGCCAGAGGTCGGGAATCATGTCGGGGGTGACGCACAGATGCCGCC
GCACTATCACCGAGAAACGCGGCGTGTGCGGGCCCACCTGGCCTCTTTAGGCCGGGCTCCGGGCTGCCCTCCAGGAAGGCCGACCTCTCCTGGG
CGGGCAAAACTTTCTCAGGGCCGAGTTCTGCCGGCACAGGTCAGGGGGCAAAGCTGGCCTCCGAGGAGCCTCTGACCGCTGGCCACTGTGTGGT
CTTGGGGCAGCTCCAGACACACAAGCCCAGAAGACCCAGAGGCTGCCTCACGTCGGAGAAATGCGCTGATCGTCTCTCTCTCTCTTTGTCTCTC
CATCTCTGTCTCTCTCCGTCTCTGTCTGTCTCTCCATCTCTGTCTCCCTGTCTCTCTCTCTGTCTCTGTCTCTCTCTGTCTCTGTCTCTCCATC
TCTTTCTCTGTCTCTCTCTGTCTCTCCGTCTGTCTCTCTGTCTCTCCGTCTGTCTCTCTCTCTCTCTTTCCGTCTCTGTCTCTCCATCTCTGTC
TCTGTCCCTGTCTCTGTCTCTCTGTGTTTCTCTGTGTCTGTCTCTGTCTCTCTCCGTCTCTGTCTGTGTCTGTCCCTGTCTCTGTTTCTCTGTC
TCTCTCTGTCTCTCTGTCTCTGACTCTCTGTGTCTGTCTGTCTCTGTCTCTCTGTCTCTCTCCATCTCTGTCTCTGTTTCTCTGTCTCTCTCTG
TCTCTCCATCTCTGTCTCTTTCTCCATCTCTGTTTCTCCATTTCTCTGTCGCTCTGTCTCTCTGTGTCTCTCTCTGTCTCTGCCTCTCTGTGTC
TGTCTCTCCGTCTCTCTCTCTCTGTCTCTCTGTGTCTATCTCTGTGTCTGTCTCTCTCTCTGTCTCCATCTCTCTCCATCTCTCTCTCCGTCTC
TGTTTCTCTCTCTGTTTCTCTCTCTGTCTCTCCATCTCTGTCTCTCCATCTCTCTGTCTCTGTCGCTCTGTCTCTCTCTGTCTCTGCCTCTCTG
TGTCTCTCTCCGTCTCTGTCTCTCCATCTGTCTCTCTGTCTCTCTCICTCTGTTTCTCTCCCTGTCTCTGTCTCTCTGTTTCTCTCCCTGTCTC
TGTCTCTCTGTTTCTCTCCCTGTCTCTGTCTCTCTCTCTGTCTGTCTTTCTTTCTCACTCTCTTTCTCTTTTAACTAAGGGAAAAGCCGTCACA
CAACCAAATGGCCATGAGGCCCGCGGCTGCCGGCTGAGGAGGGCTGCGGGAGGACCCTGAAACCCTCGCCCTCCTGAAGCTGCTCCTGTGGAGT
CCTGTGGGCGGGTGTGGCCCTGCCGAGTTCAGAGTCTGTCGCTTCGTCTGTCTGGAAACTGCAGGAGTCATGAGCAGCCTTCACGGTTTTCACC
TAAGCAATGAGCACGTGCGGGCTTTAACCCTCGGAAAATCAGAATTAAAGGATTTTACCCAAACCACTTAAACAAAGGAACTGCCTCTGCTCTG
GGCTTCTGCTGCCGAGGGCGCGATTCCGGGATTCTCATCGTTCACCGGCGGAGTCGGGGCGACTCATGAGGAGAGTGTTTTCAGATGCTGGGGA
CGTGGCCTTTCTAAGGGAGAGGGACATGGCAGGGCCTCAGGCTCAGCTCTTTGGAAACTGAAGCCCATTCACCAGGTGGGCGGGGGAGGAGCTG
GGCAGAGGTTGAGACACAAGACCTGAGAGCAGAGCAAAGTTTCTCATTTTTTTGGTTGTACAAATAACAAACACCGTTCACTGTAGAAGACTTT
TAAACACAGATAAACCACGATAAATGAGTCCTGCACCAGCGTGGCAGCTCACACCTGGAATCCCAGCGCTTTGGGAGGCCGAGGCACCAGGATC
GCTGGAGCCCAGGAGTTTGAGACCGCCCTGGGCAACATGGAGAGACCCCATCTCTACACAAAATATAACAATTAGCCAGGTGTGGTGGTGCGCC
TGTGGTCCCAGCTGCTCGGGAGGCTGAGGTGGGAGGATCCCGTGAGGCCACTGCACTCTAGCCTGGGTGACAGAGCGAGACCCTGTCTCAGAAA
AAAGAATTCGATAATCCTGCGATTGAGAATCAGACAGTGATCATTTCAGGATATGTCTTTTTTTTTTTTTCCTCCCCCTCTGTCCCAGCGCAGA
CGGCATCTCTGTCGCGAGCTTTCGGCTGAAAAAAAATCCCTCACGCCCTTCCCAGGCGAGGGAGACACAGGTTCAGGGGCATGAACGGCTCCTC
CCGTGGAAAGGACTGTGTGGCCGGCTCCCCAGGCCACGGGGGCCACTGTCCTCCATGCCACGTCCCATGCCGCTTCCACCGCAGCGTGAGGACA
GCAGGACCTGAGCCACGAGCCACACTGCTGTGGGACCCGTGTCGGGACTCGCAGGGCCTGGCAGGTGTTTCCATTTGACCTGTGGGCTCACACT
CGGCAGTTCTGGGCCCCCTGGTGTCCACCTCCATGGGAGCACCGTCCCCGGACCCCCAGAGGGGCGGCAAACACAGGAGTCCTGGGGAGTGTTG
CCGGGGCGGGAAGCCCGGACCCCCCTGTGACTCAGCTGCAGCCTCTCTGGGTGTTGCTGCGGGTGTGGAGGTTGCATCTGGCTCAGTGAGAAGC
ATTTTTAAACTTGATTCTGCAGCCACTGCAGATTCGCGGGAAGCTGGGGGGAGGCAATGCCCCACCCGGCCCCCGGGGCTGCCCCATAGCAGCC
ACCATGCCCTGGCCCCAGGCCCCAGGAGGAGGATCCTGCCGGGAGCGCCTTGCATTTTGCCTAGGACCTTCTTAACTCTCCAGACTCATCAGGG
AAGCTCTGCAGGAGCTCAGGATAAACAGGTCCTTGGCTTGAAGCCTGCCGGCATGGGTTGGGAATGGTGGGGGCTCCATGTCTGCAGCAGCCAC
GGCAGAAGCTGTGGCCACATGAGCTGGGCTCTGGGTGCGGCTGTGGCCATTGAGGAGGGAGCATGTCCCGGGTTCTTAGGTTAAGTGCGTCTTG
GCAGGGGGGCCCGGGGCTGCAGCCCTTCGTGTACCCACGTGTCCGATGTCTCTGTGCCATGCCGTGACGTTGTCCTTCACGTAAAGTTTTCAAG
ACCCACGTCCCATTGTCTGTGAAATCACAAGGTTTTACTTTATGGACTGTGTGGAATGGACCCAGGGAAGGGGGACCAGTATATGCCCAATTTG
CCAGCATGTGCAACAGTGAACTTCCCTGGACAGCTGGCCTTGGTGTGGCAGTGGGTGCTGTACGGCTGGCCTCTGTGCAGTGGGTTGGGGGGAG
CTGGATGGCTGGCCTCTGTGCGGTGGGTGGGGGGGAGCTGGACGGCTGGCCTCTGTGCGGTGGGTGGGGGCTGTACCACTGGCCTCTGTGCGGT
GGGTCGGGGGAAGCTGGACGGCTGGCCTCTGTGCAGTGGGTGGGGGGAGCTGGACGGCTGGCCTCTGTGCAGTGGGTCGGGGGGAGCTGGATGG
CTGGCCTCTGTGCAGCGGGTGGGGGAGAGCTGGACAGCTGGCCTCTGTGCGGTGGAGGCTGGACGGCTGGCCTCTGTGTGGTGTGGGGGCTGGA
TGGCTGGCCTCTGTGCAGTGGGTGGGGTTGGGGGGGTGCTGGACGGCTGGCCTCTGTGCAGTGGGTGGGGGGGTGCTGGACGGCTGGTCTTTGT
GCAGTGAGGGGGCTGGACAACTGCCCTCTGTGCAGGGGTCGGGGGGAGCTGGATGGCTGGCCTCTGTGGGGTGGGTGTAGGGGGAGCTGGACGG
CTGGCCTCTGTGCAGTGAGTCGGGGAGGGGGGTGGCTAGACAGCTGGCCTCTGTGTGGTGGCAGGGCTGGACAGCTGGCCTCTGTGCAGTGGGT
GGCGGGGGGGTGCTGGACGGCTGGCCTCTATGTAGTCGGGGAGGCTGGATGACTGGCCTCTGTGCAGTGGGGCCAGGCGCTGGATGACTGGCCT
CTATGCAGTGGGTCGGGGGGCCTGGACAGCTGGCCTCTGTGCGGTGGGTTGGGGGGGCCTGGACGTCTGGCTTCTGTGCGGTGCGGGGGCCGGC
CTCGGGCCGGTGGGCAGGCTGGAAGGTCAGCCAGGCCCTGTGGTGTCTTTAGTGAGAAACAGGAAATAAATCAAGGGGATGTTGTAGAAGTTTT
ACCGCTGCTGGTTTAGAAGTGTTTGTTTTTATTATTTGTATATAATGTGTTGTTTTCAGTCTTGCACTGGTGTTTTTAACTATCGTGTAAAGAT
GCAAACAGCAGGCCAGAAAACTGCCTTGATATATCACATTGGCAAAGACCAAGCGCACCCCGGACCCTAGTGGGTGAGCCGGACCCATTGGCAG
TGGCCTTTGTGGTGCCTCCAGGGAGCCTCACGATGCTCACGCCTCTGACCAGTGGTTCTGCGCTCAGGGCATGACCGGAGATGTGGTCTGGGAG
TTGGTTGTGTGGATGTTTGCGTTGCCAGCGTTTACAACAACAGAGGGGAACAGCCCACAGCCCAGCAGTACAATGAGGATGGTCCTTGTGATAA
ATGGGATGCTGTGATTACATTGTTTCTGAATAAGCTAAAATGCTCAAAATGTGATCTTTCTTTTATAAAAAGGCCAGGTATGGTGATACGTGCC
TGTAACCCCAGCACTTTGGGAGGCTGAGGTGGGAGGATCCCTTGAGCCCAGGAGTTGGAGAATGCAGTGAGCTGTGATCACACCCTCTAGCCTA
GGCAGCAGAGCGAGACCTTGTTCTAAGAAATAAAAAGAGAAAAAGAAAAGACAAAGTACCAAATTATGTGTCTCATTTTCTTAAGATGTATCAT
TTTGTAAAAATGTTTATAGAAATACACCCAGTGGGCTGGGCACAGTGGCTCACGCCTGTAATCCCAGCACTTTGGGAGGCTGAAGCGGGCGGAT
CACCTGAGGTCAGGAGTTCGAGACCAGCCTGGCCAACATGGTGAAACTGTGTCTCTACTAAAAATACAAAAAATTAGCCGGTGTGGTGGTGGGC
ACCTCTACTCCCAGCTACTCTGGAGGCTGAGGCAGGAGCATTGCTTGAACCTGGGAGGCGGAGGCTGCAGTGAGCCGAGATGGCTCCACTGCAC
TCCAGCCTGGGCCACAGAGCGGGACTCCGTCTCAAAAAAAAAAAAAAAAAAAATGGAGAGAGAGAGAAACAGAGAAAAACACCCTTTGGCTCTG
TGGGCGTCGGTCACCGTCCAGACCGGGTGGACGTTCGTCGGTCACCATCCAGACCAGGTGGATGTTGGATGCAAATTTCACCCGAGCTTTTTTT
TTTATTTTTTCAGTTGTGTTGAGTGTCACTTTTTTAAAAAATTGACTTATGTTGAAGATGAGTTTGAAGTTGTTGGAATTATCTCAGTCCTGAG
CTTAGGGCTTCAGCAAGCGAGTTGCCGTTCCGGGGGATGGGCTGGTCAGAGACCCCCGACTGAAGCACGCCTGCCCGAGGCTGCCCCAGGCGTC
GTCCCCCAGAAGGAAGCACGCGGAGTGGAGTGGGGTGGAGAGCAGGCAGCAGCGGTCCCGTCTCCGCAGCAGTGCAGGCTCCTTTCCCGCTCAT
TTGGGCTCTTTTGTCTGAACCAGAGACACAGATTTGTAGAGCTGCCAAATTCATGCTGAAAATTTCTAACCAAAACCAATCATCTTAATTGTCT
GGATTATGATATTTATTTTGTAAACCATTCTTGCTGTAAGCCATGCACACACTGTGTCACTAAATCTGCAAATGATTCCAGAGCCAAATGCAGT
CCTGCACCGCGGCGTCGGGTCAACGGCGGACTTCACCTATGAGGGTTCTCGTGAGATCGTGAGAGCGTAGTTTTACTGCATCTTTTCCATGTTT
AAATGTGTTTAGATTCACAAACTGTGACCGCTGTGGGGCAGTCACCCACGGTGTTCAGTGCAGCCACATGCCGTGCAGACGTGTACTGGGGAGC
CGTGGGCCACGCCACGTGGCTAGGGTGTGCACTGGACACACCGAGTCGGTTCACACGAGTTCCCACGACGACAAAATGGCCTAAGGACGCGTTT
TTCAGAATGTGTCCTTGTCATAAAGTGACACGTGGCTGCGTTTACACAAACTTATTCTTATGTAATAGTCAGCTGCAGAGTGGTTTAGACTGCC
TTATCTTTAGGAAATGTGTCTGCCTTTAACCAGACCAGCGGTGGGGGGAGGCGGCTTTTCTTTTCTTCTTTCCTCCTCTCTCCTCTCTCTCGCT
GTCTCTTCTCTGTCTCTTAACTTTCCTGGGAATCCAGTGAGCCCAGTTTTTTCACTGTGAATTTTGTTTTTGGTTTGTTTGTTTTGAGATGGAG
TCTCACTCTGTTGCCCAGGCTGGATTGCAGTGGTGCAATTTCGGCTCACTGCAGCCTCCGCCTCCCAGGTTCAAGCAATTCTCCTGCTTCACCC
TCCCAAGTGGCTAGGATTACAGGCACTCGCCACCATGCCCAGCTAATTTTTGTATTTTTAGTGGAGACGGGGTTTCACCCTGTTGGCCAGGATG
GTCTTGGTCTCCTGACCTCAGGTGATCTGTCTGCCTCGGCCTCCCAAAGTGCTGGGATTACAGGCATGAGCCACCACGCCCGGCCATGAATTTT
GTTTTTCATTTGTTTCTTTTTCAATTCATAGCAAAAGATAGGTGCAAGGTGCCTGAGACGGGCACCGCCTCGCATCATCTGCGACTTTGTTTGG
TGCCTGCCTGTCCAGGAGCAGGTGTGGCCACTGCAGAGGGGCCCTTCATTGCCCAGCCCTGCAGCTGCCCCCTCTCCCTTCCCCAGAAACTCAC
AGCTGGCCGCGAGCTCTGTGCAGAGAAGTCTTTCTCTGGAGGCTCTTGTCCTTCCTGTGTGGAGGCCTGGGTCCAGCCGCCTTTCCTTCTGCCT
GGCAGCGTTAGCTGATACTGGGTGTCCATGCTGATCACCTCACGTGGGAAACAGCTCTGACAGTACAGCCCATGAAGCTGCGTCTTGGAGATTC
CTCTGTCAGGGAAGCTGTCGTGGCTGAGTTGGGGTCCAGCAGGCACCCGCAGGGCCCAAAACCCCACAGAGCGGAGGTGGGTGGTGAGCCTGCC
AGGTGCATCTTTGGATAAATCCATTTCTGAGTTTTCAAATTACTATGAAGCTTCCAATGTCACAAAATATGTAGTCTTGTTCTTGCTAAGGAAC
ATTTTAGGTGTTTGTATTTTTAAAAATTACGTAGTTGATTCCATTCAAGCTGCCTCCTGGGCTTTTTCCTCTTTAAACGAGTCTTCTATTCTGC
CCATCGTTTTTAGTTTGTTTGTTTTGAGACAGAGTCTTGCCCTGTCGCCCAGGCTGAAGCACATGCGATCTTGGCTCACTGCCATCTCAACCTC
CCCTGGCTCAGGCGATCCTCCCCGCTCAGCCTCCTGTGTGGCTGGGATCCTCCCCACTCAGCCTCCTGTGTGGCTGGGATCACAGATGTGCACC
ACCACGTCCTGCTCATGTTTTTATTTTTTGTTTCGTAGAGATGGGGTCTCTCCATGTTGCCCAGGCTCGTCTCGAACTCCTGAGCTCAAGTGAT
CTGTTCCCCTTGGCCTCCCAAAGTTTTGGGATTACAGGCATGAGCCACTGCGACTGGCCTTGTTTTTGTTGTTGTTTGTTGTTTTTTTTTAGAT
GGAGTCTCGCTCTGTCGCCAAGGCTGGAGTGCAGTGGCGCGATCTCGGCTCACTGCAACCTCCTCCTCTTCGGTATAAGCAATTCTCCTCCCTC
AGCCTCTCGAGTAGCTGGGACTACAGGCCTCCGCCACCACGCCCGGCTAATTTTTGTATTTTTAGTAGAGACAGGGTTTCACCATGTTGGCCAG
GATGGTCTCAATCTCCTGACCTTGTGATCCACCCGCCTCGGCCTCCCCGAGTGCTGGGGTTACAGGCGTGAGCCACCATGCCCGGCCCTTGTTT
TGTTTTTAAGTCACTTTGGGTTCACAGTGTGTCCTGAGGTGTGGGGGCCTTCGGGGGGCTCGGGAGGCAGTTTCCTTTAACTTGCAGATGCCCC
TGCTCTGCGACTGCATCGCATGGAACCTGCCCTTCTCGAGAAGCTGCCCCGCATGTCCCATGTCTCTGGGGCTTGTACTTAAAACTGACGTAGA
ATGATGGTCAGCAGCTCTAGGCAATAATTGAGTCCTGGCCAGAGGGGCTGGTTCTGAAGATGGGGGAGTCACAGGCCCTGTGTGGAGGGTGACC
TCAGGAGCCCCGGCCCACGGTGCTCGGGGTGAGCCTGTGTGGTGAGGACAGTCGGCGGGCCCTGGGTCTCTGCCCACACGGAGGTGAGGACGGT
CGGCGGGCCCTGGGTCTCTGGCCACATAGCGGTGAGGACAGCCGTTGGGCCCTGGACTGCCTCTGCCCACGCAGGGTCTTGCACCTGATCTGAT
GAGCAGTGCTGGTCCAGGGAGGGTCCCACCGCATCCTCCGCCTGCCTTGGCCCCTGCAGTAGGCAGTGCTGGGGAGACAGACTGAGCGGGAGAG
GAGGCTGGGCCGGGGCTGCAGCGGCCACAGAGCCTTGGCCCTCCCAGAAACGGACTCTGATGGCAGAGGCGCCGGCCTCTTGCACAGGGTGGAA
GGTGGTGCCTGCGTGGATGGTGGTGCACCGGGGCAGGCCACCCCCTAGATGCAAGAACCCCTTGGGTGCACCGTGTGGCCCCCAGGAGTGTTGA
GACAGTCTGCCTGGGCCTCCTACAGAACACAGTTGGGAAGACACGCCGAGGGCACGGGCAGGACCTCACCTTAGCCTTTCACACCAAACATTCA
TCTCCTGAAAAGACAGGGGCCGTGGGGCCCCCAGCTGGGCTGTAGGCTCTGAGGACAGCCCCAGCACCTGTCACCCGCCAGGTCATGCAGCTCG
TGTCCCTCTGTGCAAAGTAACCGAACAGCCTCCCACAGCCCTCACCGTCGTACCCCGCCCTGCTGCGTTTGCTCCCACCGCCCTCCATGGACGC
CGTGTCCCTCCTCCTGGCCCTCAGTCAGGGCCTTCTGTTTGTCTTGCAGCAATCATGGAATTTGGGGGCTGGCGGCGCTGCGCACATCACAACT
TCCAAACCCACTATTTTATAAAGGGCCCAGTCAGTCACCTGTCTCATGGCAGCCACACAGCTGGTTCATAGCGAGGAGAGGTTTGAGCTTCCTG
GTTCTTAGGCCACAGCCCATCCCTCTTAGATTTTAAAACAGATTTTCAATGAGTTCTAAAACCTGTCTTACTGGAGGGTTAAGTAACTGATAGA
AGCCTGTCATGAAGGAAGAGAAGCGTGTTAAGGTTGTGCTTGGAAAATGTTTCCATCTTAGAGAACTGGCCGGGCCGTGGCCACAGGTCACGAT
GAGTAAGCACTGCATTGACACAGGCTTCTCCATCTCCATGTCAGGACGTGCACTCGCCGATGAAGTGTCCACCCAAGATGGCGACGGGTGAGCA
CGGGCACGTGTGGCCCGGGCGAGGTCTGTGTGGTGCTGAACGCCTCCTCTGCTCTGTTCCCTTCCAGCCCGACCCTGCCCGCCCTGGACTGGCA
GCTGCCGTCCCACTCAGGCCCGTATGAGCTTCGGATTGAGGTGCAGCCCAAGTCCCACCACCGAGCCCACTACGAGACGGAGGGCAGCCGGGGG
GCCGTGAAGGCGTCGGCCGGAGGACACCCCATCGTGCAGGTAGGCACTGCGGCCAGACTCGCACGTCACTTGGTGCTTTTGTGGTCAAAAAGTA
ACTTTGGAGCCACAGCTAACTGTGCTTAGACTCTCCCAGCCAGGCCAGCTGAATGCTCTGTCGTGGTTAGTCCCGGGCGGCTGCTCACCGCCTC
TGAGCTGGAATGGGAGCATGCCACAGCTCCTCCTGCTTCCACACGTGCAGGCCCTCTCTCGTTGTTTTCAGATTTGCCCGAAGCAGCTCTCGTA
TCCGTGGATGCTGCCTGTGGTGTGGATGTTTTGGGGGTTGATTCTCCCCTCTCTAAAGCCTCTGAACTTGGAGGCAGGAGTTCTCACCAGCAGC
ACCAGCCTCGCCTGGTGGGGGACCCTCAGAAATGCAGGTTCTGTGGCCCACCCAGGCTTGCAGGATCCCATACTCTGGGCTTGGGTGCTGGGGG
GCACCAGGGCTCAAGGTGCTCCCCGAGGAGAGGAGGAGTGGCTTCCAGAGCGGGCCTGGGCCTGGCAGCCACATCCCTGCCGGGGTAGGCCTGG
GCCTGGCAACCCTGCAGCCGCGTCCCTGCTGGGGTGGGCCTGACTCCCCCGGGCTGTGGTGTGGCTTCCTCACCTTCCTCACACCTTGCGGGAA
AGCCACGTGGAGTTAGGCTGTCGAGGGAGGGGTGTAATGACCGCGCCAGGGACGGGCAGCGTCTGCACAGAGCCATGCTCCCTCTGCACCCCAG
GTGTTGATGTGGATCCCGGGTAGCCGTGGGTGTTGGCATTTTCTTCGTTCCACCTGTTACCTCGTCAGCTATAGGCCCCAGGCCCTAGATCTGC
TGCTTTGGCAGCCACAGAGCAGCAAGTGTGTCCAGCCCCAGGGCTCCAGGCTACGCGGCAGCACAGAGGATGTTTTCCGAGCACACCCCAACAG
CAAAAAACCGGGTGCCCCAGCCCCGTTATTTAGGGAATGATGCAAGCCGGAAGGGGTGTTCAGCAGAGTGCCTCGTATTTTTATTTTCTTCCAT
TTAGATAAATAGTGGAGAGAAGGTGGCCGCAGGGACAAATGGCTGTTTGCCAACGCAGCAGCCGGCGGCAGGGTAATTAGCGGGCAGGGCGTGG
GGTGAGGCGTTTTCGCCAGCACTTGCAGATGATGTTAATGTGATGTTTGTGGCCTGTGTGCTTGATGCCAATTAGTACCTCCCTCGTTGGTGCT
TGAAGCAAGGAAATGCGGGAGACACCTGAGCGCACAGTGGCCCGTTTGACATTTAAAGTCTCCACCCTCCCAGGTGGGAAAGGTCAGACAGCGC
TGCACCACGCTCCGTCACTGCAGGCCTGCACCTCCCCATCCCTGCCTCGGTTTTTACTGACCGAAAGCTTGTTATTGCTCAGTGGGAAGGATAT
TAGATAGGACAAGAAACGAGGCTGTGAAAGGAGGAGTGGAAATAAAGCAATAGAGAAATAATGGCCCAGTCCACCCTATTTGCTGTAAATTATG
TCGGTGAGGGGCACTGACGTCGCCCTTTTCACTGTATCTGGATGAGGGGCGGGTGTAGGAATCGAGGCTGTTACTTGGAGGGTAAAGCAGGACC
ATGAGTGTGGAGAGAGCTCTGAGGTTTCTGTTTGTTTTGTTTTTTGGTTTGTTTGTTTTTTTTTTTTTTTTGAGACGGAGTCTCGCTCTCACCC
GGGCTGGAGTGCAGTGGTGTGATCTCAGCTCACTGCAACCTCTGTCTCCTGGGTTCAAGTGATTCTCCTGCCTCAGCCTCCCGAGTAGCTGGGA
CTACAGGCAGCCCTGGGTTTTAATGGATGTATTTTCACCTGCCTGTGAGTAGCGCCGGTGCCTGGTGCCGCCCGTCTCTGCGGACACAGCTCAG
TGCTTGGGTACTCTCCCTGCGTCTTCTGGAATCTTCTGGAATCCCGTCTCTGGTTCTGCCATCTGGGTGTCTTGGGCTGAGGGTGCAGAAATCT
ATCAATACAGATGCCACTGAGAGGATCCAGGTGTTCTGGAGGACTCAGGAAACTTGAAACAGTAATTAAAAATATCTTTAAAATGTACCCTCAA
CAATTATTCCAAATCAAGTGGCCCGTGTGTGCGCAGCAGCGACACGGGGAGAGGTGGCTCTGCGTTTCCACAGAAGGAGCCCTTTCCATTGAAA
CGCGGGAGAAAACGGTCCCCTGCACGGAGAACGTCAGGAGCTGTCAGCATATCTCATTTGTAGAAATTAGTAATTTAAAGAGTTTTGTGATGTA
TCTGTGGCAGAGTGGATTTCTGTCTCTGCAAGTGTGAGGTAAAAGCAGCTAAAAAGAGGGGGTTTTGGGGGTTAGTTTTCTCTTTCAGTTGGTG
GACCACAAATTGGATAAAAATAGAATTTGTGCATAATTTAAAAACAAACGAGAATAGAAAGCTCTCAGTTGCCGTGGAAGCTCCTCGTAACCGC
AGACGTGCCCTCGGTGCCGTCGGGTTATTTTAGTACATCCTTTGACGCGGCCCCTTTCCCGCTTTTCAGAAACGAGGCTCATCGCACTGGCCTG
GGGGCGCGAGGACGAGGCCGTGGGTAGTGGGCGCTGCGGGCGGCTGGTCAGAGCCACTGGGAGCCGTTGGGAACAACTGGCGCGGGAGTTTCCT
GCATTTGAGAAATGCTGTTCTCTTCGTTGTGSAATCGTCTGTCCGCTGCATCTAAAAAGCTTGTCCAGAAAATTAAAGCCCGCTGTCTCCTGCA
GTGCAGCTGAGCCCTGGAGGGTGGCGGGGGGCCGGGGGATGTGGGTGTCAGCTCTCGCGGCCAGGGGCACGCTGTGCCCCCGCGCCCGGTATCC
CCGTCCTCCCGCACCCGGCGTCCGCGTCCTCCCACGCCCGCTGTCTCCGTCCTCCCCATCCCCATGTGTTTCGCAGTTGGGAAGTGAAGCACGT
GAGCAGAACGCAGCACCTTCTCTCCAGGAAGCATTCGGAGCATGGACTCTGGGTTAGGAAAGGGTGTTTGCTCCTCTGGATAGGTGCTCGCTCT
TACTTGTGGGTGCTGAGGGGCGTGAGGGGGTCCGGCATCCGGGTACCTGTGGGCGCTGAGGGGCGTGAGGGGGTCCGGCATCCGGGGCTTCTCC
CATTTAGGGCCTTTCCTTCAGAGGGAAGGAGGCCAGCCCCCTCCAGTGAGCACTGAGCGTGCCCCCTGCCGTCTCTGTGGGGATGTCATTGTTC
CCACGCCGGGGAGAGACCACCCCAGGCCAGCCCCTATTCTTCTGGCCACATTGCCATGATCACACCGTCCGCACCCTGAGGCCACCCCCATTAC
GGCTGCCACGGCGCTAAAAACACCAGCATCAAAGCAACAGGGCCGTGGTTTAGCCTGGAAGAGCGGGGCCCTGGAGCCGTCTGCGAGGGGGTCT
GGGTGGTTCAGGATGGCCGGCAGCCCCTGGTGGACGTGGTCCCTGCGGGGTCTGGCGGGACCTCGGCCATGGTGATGGGTGCGCCCCCTCTGCA
GGGTGGGGCCTGCGGGTAAGGACAGTCCCGCCCCCTGTGCGTGTGAGGGGATAGTGGAGAGAGGGGTCTGGCGTGGGCCTCACTGCTCCTGGGA
AACCAAAGATGAAGACGCCTGCTTAGGACTCACCTTGCCCTGGTAGAACAAGTGACTCAGACAGAGCCTCCCAGAGGCTTCTGGCCAGGCAGGG
AGTGACCAGCAGCGCAGCCAGCCTCAGCTCCTGCCCACACAGCCCCCTTGTCACCGGCTCCTCCTGCCCCTGCTCCAGCACCTTGGCCACCAGT
TCTCGGTGGGCCGGTCTGGATCTGCCGATGCCGGGACCTCGCCAGGCCGCTGGTTGCAGGGGAGGCTGAGGGGGCGTCCGCCTGCTGTCTGCTT
CTCACCAGCATTTCCAGTGAGGCATGAAAGTGATGCGTGGCGGATGTTTCCCTCTGGCCTCAGCTTTCCCCCAAATCCCAGCTCACACTGAGTG
TCCTGATGGTGGCACCTCGGGCAGCCGCCACCCTCTGCCCTCCACCCTCTGCCCTGACGGCAGGCTCTGCCTGAGGGGGAGGGTCTCTCTGTAC
ACACAGTCTCCTCACCCTGGAGGGCCTGCTCTGCTCTCAACGGAGCTGGAACCAGGAGTTCGCGAGTGCTCTGGGTGGCTGCTGGCTTTTCTCC
TTGTCTCACCTCCTCATCCGCACACAGCCTCTGCCCTCAGCGCTGCTCACCTCGGTATGTAGGCAGCCGGCATCAGGATGGTCTGGGGTGAGAC
AGGCAGTGTTCTTCCCCTTCTCTGGCCAGGGAGGGTGGAGAAGCACCCGGGCACAGCCATTGAGGCCTTTTCGTGCAACTCATTGCCTTTCTGG
GCGTGAAGGTGGAGGCTGGTCCTCCACCTGCGTGTTCTCCATCTGGGGGACTGTGGTCCCGGAGGAGACCTGGAGTCCGGCTCCCCGCTCCGTC
CGAGGAGGAGCTGAGCGGCGTGAGCAGTGCCCGCCCTGGTCACTCTGCCTTGCTTTCCTCCTCTCTGTTTCTGGTTGTGCCTCCTTCCTTCCCT
CCTGCTCTCTTTGCCGTGGACTGCAGCCCCCTCGCCAGATGTGTTCCTCACATGAGTCAACCCGTCCTTGGCCTGCGTCCTCCACTGCTCGTGG
CAGCTACACTCTCCCGGAGGTGGCCGGGAAGGGCCCAGGGCCTTGAGAAGATGTCTTTGCAGCACGTCCCCGGCCCTCGGCCTCCACCTCCATG
GCACCCACCATCCCACGGGGGCACGTGGACCTCTGTGGGATATAGCCTGACCTCTGACCCAGGGGTCAAATAACTCAGGTCTGTTTACCAAGTC
ACCAAAATAACCTGCCCGATAGCATCTCACCCATCACACGGGAGGCCGGGCTCGTGTGAATAATTAATAGGTCACTGGAATGTGAAACGAGAAG
CCTCGGCCCGTTCTGAAACAGCATTTTAGGTTTGCAGCTTATAATATTGTCAGTTAGGAGAAGGGCCAACTGAGTCTGCAAAGAGGTGAGGGCG
GGTGGTCTGGGATCCTGCCTCAGCCGCGCGGGTGAGAGGGGCGGGCGGGTCAGGCGAAGACTCAGAGTCAGGAGGCCAGACTGGCCCCAGGGAG
GGCTCCTGGCCATACCCCAAGCTCAGGACGCAGGGTGGGGCGGGCCCGCTGGTGCCCACAGTGGCCTTGGTGGGCCTCTCCTTCCATTGCCCAG
CTTGTAGGTCAACTTTGCCTCCACAGATCCGTAACAGACGTTTTTTCTGTCATGTAAGAAACTTGACAGCTGAAGACCAAGGAGTGGAGCCTCC
CGTGAGGCAGCTCTTCTTTTCCGGTTGTTAAAATGCGCGTCGGGCCAGTGGTTCTGACTAATTCTCACTTTGTGTGGTACCTTCAGCTGGCAGG
GGCTCGGGGAGTGTTCAGTACCGTGAGGAGCAGGTTCCTGAGAGCCCGTGAATACCGTGGACAGATTACCACAGCACGCACTGTTCCACGCAGG
GCCTCATCCACGCTTTCTTAAGTGGTTTGCCACACACAGGAGCTCCTGGTTCTCCATGTCCCCTGCCAGGCAGGGCTGTTAAACTGGAAAGCCG
GGGAGGGACGTCGCTGGGACACATTGTTATCTGATCAGAGCTCCGACGCCCCACCAGGAGCTGTGCTATTGCAGAACCTCAAACAGCAGCCGCT
CCGGCCCCGCCCCAGGAGCCACGCAGAGGCCCACGCACGGGGAGGACGCCGTGGGGTGGAAACTAAAGTGCAGGTCTGACCACAAAACCACACG
CCACGGCACCGCTGCTGCCAAATGCCGGAGGCCTGCAGGAGGCACGGATTAATTTTTTATGAGTCTGCCTTGGAACCTAGACAACAGCCCTCTC
CTCTTTCAGCACAATTTCAGGGCGAATGACCGTCACTTCCCTGGACTGCTAAGCGCATAAACGCACAGGGAATTTCCTAGTTCCACATACGCCG
TTGTTCCCGCAGCCCATGCAGCTGGGAGAGCACACATGGTTGCCGTCGGGCTGTGTCTGTTTTCCCAGGAGCAAGAACTCTCAGAAGTGCAGTG
TTGAAGCCCGAGCCTTCCCCGTGTCAGCTTTTATGATGAACGAGGATTCACCATTTATTTCTCCCCTAAAGCAAAGCCCACGGGCTCTGTTTTG
AGCGATGGGGACAGATGCTCGGTGCTGCTGGGAAGGTGTTCACGGGGCAGATTCGCTGTGTCAGGAGACCAACGTGGAATGAGGTCGGCCCAGC
CAGTCACTGCTTAAAAACAGTAATGACAATATTTGAGAAAATGGGTGGAATTTTGTGGAGATTTACTGGCACGGGGAAGGCGTGTGGCCTCTGG
CAGGTCTGCGACACTGATTTCTAACTACCTGTTCCACAGCTCCACTGCCTGGAAAAACTGGCAAAAATGTGGCCTGGGTCTCCTGACAGGCCTG
GCCATTGGACTGGGGGTGTGCAGCTGAGACAGGAAATACGAGGCGCATGAGTGTCCCCAAGCCCAGATCCTCAGCCTCCAAAATCTTCTATTAG
TAATTTCAGAGCTTTGGCTTCTTGGTGCTTTGGGAAAATTTAAGATGACGCGTGGGCTCTGGCACGGCCGTTGCTAGTGACCTCTCCTAATAGC
GCAGAAGGGCCTCCCAGGAGGCTTTCCTAGGTAGAGAGGGGTCTGCACGCCCACTGGAACATGCTCTATCCCAGCCTCAGAAAACCCACATTTG
GTCCAGGTGGGACAGGAGGAGTGGACACGTCACCTGGAGACCACATGAGAAGCGGCCATCTGCAGCAGCAATCACCCCGTGGGGAGGCGGCGCC
CTGGCAGCTCTCATGTGGGGCCTGGTCCTTCCTCGCTGCCTGAGCAATGGGCCCTGCTTCCCCGCAGACACCGTGGTGCCCCTGGGCCACACCC
AGCAGGTGTCCAGTGACCGAGGGTGGTGTTGGCCATGGCAGGTCCCTGCCCGTTTCTCCGAGGACCTGGGGCTGTGACCCAGCTGGGTTCTCCC
GGGAAGGAGCGGCCACTGTCTGCACTGTCCAGTGCTGAGGGTGGCAGCCATTTCTGAAGCCTCCCAGGTTTGTGCCCTGCTGTGTCTGAACAGC
TCCGGAGTGCAGGGTCTCTGTGTCAGTGCCGGTTCTAAGAGGAACGAGATGTGGTTTCAGAGACACTCCTCTTCTCTGAGGAGCTGCTGGGCGG
GACGGCTGCTTGCTTTCTTTGTCTGTGACGTTGGTGGCTGTTCCTTGCCCAGGCCCTGCCCAGGCCAGACGTGGGAGTGGCGGCTGGGAGCACC
CATGGGCGTGTGCCCGTCTGGGCTGGGCTCGTGTCCGTGGACCTGCCTCAAGCCACACCTCCCTCCTCCAGGCAGGACACCGGGCCTTGTCTGT
GGACACAGGCGCTTTGCTGGGAACAGCACAGATGGCCCCTGGGCCCTGCGGAGCCGAGACGCACCATCATACACACTTGGAGCACATAGACTGA
CCAGGTCTCACCCTGCGAGGCTCCCTGAGGCCATGTCCTAGGGAAATAGAAGGTTTCACCCGGAGATTGGGGAGTGCAGGGCTGTGGCGCTGCA
GGGTGCGGGCCCTGAAGGAGGGGCAGGGGTGCTGCTGGATGGTGGCTCTTCCACCCCAGGATTCCTCGGGCCCTTCACCCCTGTGTGTCTGGAC
TGGGTATCGCTGTAGCTGTCGGGCTCTGGAGGTGCACATGGCTGCTGGGCGGGCAGCGCTCCTGGGCCTCTCTCCCCCGTCATTTCGGTTTTGA
TGTGTTTGAGCCACTGACCAGCAGGCTTAGGGGCCATTCTGGAGCTGGGAGGGCTTGTGGGGGAGGCAGGATGAACAGGGGCTGCTTGCCGGTC
CTCAGGCCACCCTGCCAGCTCCTTGATGTGTCTCCCTGAGATCAGCCAGGGCATCCCGGCTGTGGGTGGGCTGTGACCTGAGCCCGTCAGGCCC
TGCTGGGGTAGACTGGGGAGAAGTGCTGAGCGGAAACCACCTCACTTCTGACAAATGACAAGCGCTCCTGCCGCTTGGTGTCAGGCGACGCGAC
TCTCAGTGGCACGGATGTCTCTAGAAGGTTGCCGTTTCTCCCTTCGTGGCCGTGGGCTGTGGATTCTGTGTGCTTCTGGCCCTGGGGTCACGGG
GTTGCGGCGTTTCCCACTTGCCTGTTTCAGGAGTTGCATTCTAGATGATTCTCTCTCCATAGAAGGTCGAGGTTTCTCAGCATGGATTAATTGA
TTAGATTTAATTAAATCTAAACGTGGTGTTTCCGGGCAGAATCTTCTTCAGCCAAAGTGAGCGTGGCCGTCTCTTCTCTGGGCGCTTCCAGGTG
CGTTGGGGCCCCTGCACCCTCCACGTCAGTGCCCTGGCTCCGTGGCCCTGTCGGTGCTCTGGTGAGTCTGGGGTTCGTGCCGAGCGCCATTGCC
TCCAGGTCGAGGTGGGAAGCGCGCTGGTGGGGCAAGGTGGTCCCTGCATGGCACAGGTTGGGAGAGCAGCCGGGAGGGCAGCGGGCCGAGGCGG
TGGGGGAGGGCACCAGTTCTCCACGGCAAGCACGGCCTCTGGGGCTGCTCTTCCGGAGAGACGTGGAGACCCGCGTGCACGTGTGGAGACTGGC
ACGTGTGAGGGGCCCACAGGTGTGGCCGGGCGGCCTTTCTCCTCCCCACCTCTGTCCACCCTCGCCGAGGCGTCGGTCCTGCTGAGCAGCCTCT
GCTGCCTTCGTGCAGGTGACAGCGCACGTGCCATTCACGGGCGCCATGTCTCCAAGAAGAGGGGCTCCTGCCGCGTGCCTGTTCTGGGGTTGCC
ACTCAACATCCTTCCAGCACCTTCTCTGGGCTTCCGGTTTGAATGATCTTTCTCCAAAGTCGGCGGAAGGGGCTAGAAAAGTAGGGACCTGGGA
AGAAAGCCCAGACACCTTGGAGCCGGGGTCCGGGAAACAGGCCCTGGCAGCTCAGCGCTGCTTCTCTGGAGTGGGGATACAATGTTGGTTCACA
GACGTGCCGTGGCGGCGGCGGGTGGTGGCAGCAGTGTGCGGTTGTTGGCGCCACGTGCTCACGCTGGGTCCCTCCTGGGGAGCTTCTGCTCCGG
CGGGCCACGCCTTGCTGTAGCCTGCAGGTCATCTGGTTGGTGTCTGGGGGACGTCTGGGGCCCCAGCCCGGAGCTCAGCTCTGAGCATCAAGAA
ACCCGAGTTTCTCCCCACTCCCACCCAGACAGCTCCCCACTCCCACCCGGACAGCTCCCCACTCCCACCCGGACAGCAGCAGCTCTGTTTGGAA
GGGGACACACCAGAGCTTCCCTTGTAGCAAGTGAGGCACGCTCAGCTGGAGCCTGGCAGTCCTGCAGCTCCGCCTGCCCCCTTTTGAAAACCAA
AAATCCCAAGAGACTTATGTTGAAACAGCCCTTCCCGGTCAGGACTGAGCCTGTGGCTGAGTGCAGTTTTTCAGTCGCGGCTGGAAGGTCCCTG
TGCGGGGCGGCGAGTCCCTCTAGATTTGCTTTACAGCCACAAATTCAGTTTTATCTGTGGATCTGAACACGATCTATGAATCACTAGGAAAGCA
ACTGTGTTCACGGGAAGGATTCTCAGAATTGAGCTGACACTCCGTGGTGTGAACTCAGTATTGTCAGCTTGTGCACCGCGCGTATGGACACACA
CCTGCACGCACTGTCACACACACTTGCCTCGGGCCCCCACACACACCGCCCTGAGAGCTAGGAAGACGAATCGGTCAGTCACACGGATTCTGCA
GACAGCTTCCTTCACCAGGCATGGTAGTGAGGGCAGCGCCCACTACTCACCCAGGGCCTCTCGTGCAGGAAACTGCATGGACACGTCGTCCTGT
TCCTGTCATGGGAAGGGGCCTGGAGCCGTCCCTCGGGGGGTGGGGAGGAGCTAGTTTCCCAGTCGGCGCCCGTGCATTCACCGGTCTGAGAATC
CACTCAGCAGGCTTGCTATGTAACCTCCGCCGTTCAGGCCTCAGCATGCAGCAAGCCCGTGGGGTCCCGTCTGCCCAGCCTCTGCCTCGGTTCC
TGCCGGGCACATGGACGCTTCCCTTGAATCCTTCTGCCGACGGCATCTGTGGGAGGGCAGGTACGATCGATCCTGGTCCTGTCCCCCTAGCCCT
GGGCGGGTGTCCCGGCACCTCCAGGCACAAGGCCCCTTCCCCAGGCTGCCCAGAGACCCCAGCCCCGAGGTGCTGGTCCGCCCACCCTCTCTGG
GAAGGGCTGTGCCACATGGCGAAAGCTGCTGTGGGTCCTGGTACCTGGTGTCCCTGCCTGCCAGGGCTCATGATGCCAACAGTGCCAGGTTTGT
GGGATTTGGGGGTCAGGAGGGCCTGGCCCTGCTCCTGCAAGGCCGAGATCTGTTCCCTTGGAGGGTTGAGGGAGTGTTGGCTGTAGGGTTCCTG
GAGGCCTCTCTCATGCACTCTGGGTCTGTCAGGAGGCTGGCAACTGTGGCCAAACAGGTAGAAATAAAACCTGAAATGAGAAAAAAACTTTTTT
CTAGAACTTCCCAGAGAGCAGTGAGCTGGTGCCAGTGGGTCAGAGGCTTGTCACATGTGGTGTGTGTGTGTGCGTGTGTGTGTGCACGCTCTGG
GGGGGTGTGCACATTTCCCAGTGTGGCTGTGCGTGTGTGTGTGCCCACGTGTGTTCACAGAACACCAGGCTTTGCCCCGACTCCTGGCTCAGGC
CTGAGTCTCCAGCCATGGCGTCCCCGTTCCTCCCGTTCCTTCTCTGCACTGCACACTGGGAACCAGGACAAGAGGTGTGCGGCGTCAGGCTGTG
TCCTTCCCCCACCCACCCCGAGCCCACGCTGCTCTGGAGGGAGACCCCAGCTTCCCCCGCCTACCCCGAGCCCTCGCTGCTCTGGAGGGAGACC
CCAGGCCCCTCAGGCCCTTTCCCGCATCTGAAGGGGCCAGGGCAGGGCGTCCGGCAGATGAAGCTGCCCCTTAAAGCCAGGTCTGGGCCGGTTT
ACAGGGAAGCACTGCTCACGTACAACCTGGCAGACCACACCAGCGCCGCACACACAGGCACCCCTGTGGGCACCCACGTGCCCGACCCCGCAGA
CCACACCGGCACTGCACACACAGGCACCCCCGTGGGCACACACGTGCCCGACCCCACAGACCACACCGGCGCTGCACACACAGGCACCCCCGTG
GCCACACACATGCCTGCACACGCACACTTCTGGTGCACACGCCCACACTGACGGAGGCTTCTGCACACCACACAGCCAGCAGGTGAGGAAGTGG
ACGCTGCGTGGCTGTCTCTTGGTGGCTTCACCTGGAATGTCACCGTGGACCCCAGAGCCCGAAGCCCCGGCAAGCACAGAAATCACACCGGCAG
CCTGTCCCACTTCGGGCAGGTGAGGCCTGGCTGAGAAAAGGGGGAAGGAGAGGGAGGATGCGGGGGTCTGTCCTGGTCCTCCACCCGTTTCGGG
TTTGGGGTGCTCCCTTCGACTACTGGCTCCATTTTTGATGTCTTTTACTTTTGTTTCGATTTTTCCTGGTATCTTGAGTAAAGTCTCCCTGGAG
CATCAAATTGGATTTTTCTTACAAGTACCGTGTGCTGTTGACTCATCTGCTTTGGTGTGCCGTGGGTCACGTGGGCCTGGGCTGTGGGTAAGGT
GGGCCTCGTTCCTGCAGGAACACCTGGCTCTGCAGCAGTGGCTGCCGTATCATCCTGGCCAGGCCCCTGTACCGAGGGTGGAGCCACGGGGTCT
CTGATGCCTTCCTTCTCGTGCTTTGGTGGAGAATGTGGCTGGACGTCCTCCCATGACAGATGCACTCTCCTGCCTGGTTCCCTGGCCCCGGGCC
CAGCCCTGAGAGACGCACCACGGCCTTCACCTGGAAGTGTCACCTCTGGCTGCTGTTGTGGGTGATGAGGATTCCAGGGCTGAGAAGAGGACCC
TAGACGCTGTTCACACAGCGTGGCAATCTCTCAAATTAATCCAGCATGGGGGGAATCGGGGTTGAAACCGGTTTGTGAGCCGGATTTTCTGTCC
CTGTGTACTTCCTTGGGGCAATTTGAAAAATGCAGATTTTACTCGTAGGCCCTAAATGGATAATTTCCCTTTAGGCTGACGGCTGCTCCTGTGC
CCTGCCTGGAGAGAAGCCTGACTCCAGGTCGCCTTGGCTCCGTTTCTGTGCAGCAGCTCAGACTATGGGGTGGAGGGTGAGACATTCCCACCCG
TGAGCTGCCTCTGTCCACCCCACACCTCCCTCCCCTGGGTGCACGCAGCTGAGTTCCACGTTGAATAAATTTGGCCTCAATCATCCACTACCCA
CCATGTCACCGTGTAAGTGCTGAGGTCAGCACAGGAGCTAAAAACAAACATTGTAACGTGTAGGAGACTCAGTAGCTCTTATACCTCGGCAGTG
GAATAAGAATAATACATTTTGTTTAGTTAAGGTTGAGGCAGAGGGAGGTCATTGTTGTGACAGGGAATTTTATAAATAGGATTCTGACAGGTTC
TGTACTGTGCCAGAGGCAGAAAGCCCATGCAGTGGGGCCCGTGACGTATTTAAATGACTTTTCTTAAAATGAACTTCATGTTTTAAATAATAGA
TGTTTACCTCCCAGGTGAAACCTTAAATATTCCCATTTTCACTGAAATGGCATGTTTTTTAAAGGATACAGTCATACCCCAAATTCACAGCATT
CCACACACCATCAGAGAACTGCCACAGGGATCCTGGGGGAATTTTAAATTGTAGTTTTAAGACCACGTGACCCTGGAGCTGCCCCGGTGCCCCT
CCCCCACCCCCCGAGGTGGTTTTGGTCAGCGACTCATTTACATTTTTTTCTATGTAAATTAAATTTTTAGGTATTAAATCTCGGAAAATGGAAT
ACTGCGTGGCAGAATCCGCCTCTTAATCTGCTTTTCTTTTTACATTTCCCATCATAAAGATGTTTGAGTCCCACAGAGAGTTAAGGTATCTCGG
ACTGCGTGGCAGAATCCGCCTCTTAATCTGCTTTTCTTTTTACATTTCCCATCATAAAGATGTTTGAGTCCCACAGAGAGTTAAGGTATCTCGG
GAAGTTCATTAACTTGGCAAACGGCTGGAGTCTCTCTTCCGAGGAGCTGAGGGCTGGACAGGCAGGTCCCAGCGTCCCCCGTCCTGACTGTTTG
TGTGGAGACACTCATGTATTCATATGGGAAGGGGGGCTCTATTGGAAAGGGAAGGGCTTTTTGTTCTGCAGAGTTACGTGGTCTTAGCAGTAAT
CACTTTGGAGTGGGCTGCTGGTGGCCTTGGGTGCAGAGACTCCTGTGGGCATCTGCGCACATGGGGGCTGTGTCCTCATCCCTGTCTGTGGCTT
CCCCACCGTGTTCCCACGTGATGCCTTCGTTCAGCCTCTGGCTTTGTGTTTCAGACCCCAAACGCCCCTTCGATGTCTCAGCTGGGACCTGGGT
CCCGGCTCAGCCTCACGCGGCGTCCCAGTCCCTGCCCATCAGTCTGGGCCTCACGGTCAGGCCCTCAGTGACAGCAGCCGTCGCTTCACTGTGG
CCTCGTCCCTCGAGGGCTCTGAGTTCAGCCGTCGCTTCACTGTGGCCTCCTCCCTGGCGGGGCTCTGAGTTCAGCCACCGGTGAGATTGGTTTG
TTCTTTACTGTGGTGACCACACGGCGGCTGAGGCTAAGCTGGAGCCACGGAGCAAGGGCCGCCTCTTCGCCTGCCCGGGCCCCTCTAGCCCCAT
CTTCCCTCTGCCCACTGACATCTGAGAATCCGCCGTCCCCTGGCCCGGGCTCCCCCGCCTTCTCCCTCGCCTCGCTGGCCCTGAGTGGGCAGCA
GGCATCTGCGTGGTCAGGGGCAGGCGTCGCATTTTCCTGTGGCTGGAACGTGCCTGCAGGAGCCCAGGCCTGGACTCTGCACGTGGTCATCGTG
CTGCAGTTGCCGTCTGTGGTCAAAGATTTTAAAGTTAGTTTAGTGAGAAAATCGACTCCAAACGTGATCAGAGGGGTCGACGCGTCCCAGGATG
AGTGGTTCCCGGGGCTGGGCGTTGCCACGGGCCTTGCATGGATTTTGCTGATGAATCTCAGGGCTCTGAGGCTGGCGCTCATCGGGCCCCACTT
GGGGGATGCAGAAGGGAAGGCACTCTGAACGAGGGGCCCTGTGGGAGGCCCCACTGCGCAAAGGGTTTGACTCCTGCGGCCTCCCTCAAGGCCT
TTGTGCTTCCTTTGAGAATCAAACGTTCCTGTTCCAAACCAAACTTGCCTCTTGCATATGGAATCAGACTATAGTGATTGTAAATGGTAGCGTT
AAAGATAAAAACAAACAGTTCTAAAAATGACTCACTGCTGAAGGCGGAACCACGCGTGGTGCAGAGAAGGAGGGTGAGAGGGAACGGCACCACA
CTCTGTGTTTACCTCTCAAGACGCTGATGAGCAACTCATCCCACAGAACAGCTGGTCCTGAACATGTGTGTGCCCTGCCGTGAGGCGCACACCT
TCCCAGAGGCGTGAGTGGGGCCCGGCCACTAGTGTCCTGGCGGAAGCACCGGGGCTGCTCCATTTGTCTGGAGTCCTGTCCCAGTGGTGTGGGT
TTTGAGTCTCACCCACACGGCGCCGGCCGAGAAGCCATGAGCGGCTCTGCAGGGTGGTGCGGACTATAGACACCTCCATGCATGGTTGCCCTAG
GCAGTGTCTGCCGCCAGCGTCTGCCTCTGGACACTAGCTGCTCAGCCACCAGGACGGCGTTGAGCGCTCCCCTGCCTGTGCAGGAGGAGAGGGG
CAGGGAGCCATCAGCAAGGGTGCTGGGGCCTGGGGCCCGTCGTGCTGTGTGTGTGCAAACGCAGTGTGGCGTCCCCTGATCAGGAGTGGGAGCT
GCGCGCTCCCCGGTTCCAGGTTAATGGAACACAGGAAGGTGGGTCATGTCCTGCGGGGCCTCATCTGGATTTATTGGTGAAGCGAGCCTTGCAC
ATGGCTCAGCGAGGGCTCCGCTTTCCTGCACACGCCCCCTGCCAGCTTCACTGTGGCCTCAGAATGGGCACACACAGCAAGTGTGGATGCATAC
GTGCAAGGCCCGTTTCAAAGCCTAAAACTCAGTGCCCAGGTCCATTGGGATAACAAGGCATTTTCTAATCTGATTTACAAAGACACCAGTATGA
CATGGCAGAGAAATAAAAAGGGGGCACGGCAGGGGGACACAGGCCTCGAACCCGCCGCAGGTTTTCTCTGCGTTCCGGTGACTCCCGGCGGTCT
GTGCTCTGGGTGCTGAGCAGGTGTTTTCTGTTCTCTCGCCAGCTGCATGGCTACTTGGAGAATGAGCCGCTGATGCTGCAGCTTTTCATTGGGA
CGGCGGACGACCGCCTGCTGCGCCCGCACGCCTTCTACCAGGTGCACCGCATCACAGGGAAGACCGTGTCCACCACCAGCCACGAGGCCATCCT
CTCCAACACCAAAGTCCTGGAGATCCCACTCCTGCCGGAGAACAGCATGCGAGCCGTGTAAGCCGCGGGGGACCTCCGGCCTCTGGCAGGGGGC
GGTAGGAGGGGGCGTGCCTCCCTCCCAGTCCCGGGGGGTCTGGCCAGCCCCCCGCACTTCCAGGCTGCGTGGCCAGGACACTTTTGGTTTAACA
GCCAATAAGTAAACTTCACTCTTGCTTTTGACAATGGATTTGCTTCTCTCTAGGGTTTCAGCCCTGTGTATATTGCCACTTAAAATGTCAGATA
TGCAGCTATAAATACCTCCCTCTGCATTTAGCTTCTAAACAGGACCCCTATTTCTTTCCGAATACAGCAGTGTAATGAGAGGATGGTTAAAACG
TGAGCATCAAAAAATTAAAGCAGTCAGCACATCTCGATCACGTTGACCTTTAGGAGGTTCCGACAACACGGATAGGCCTGTGCTGATGTTCTGA
GTCTAATTTGACACTGTAATTTGTTTAAAAGTGAATTTTTTAGTAAGGTGAGGACGAAACATGTAACTTCCAAATAAGGCCTTACTCACTTAAT
CCTAGCTTGTGCTGGGCACGCTGGCCCTAACCTGTGAAGGCAGCCGAGGAGGAGGACTGCTGGTTCCCGAGAGATTGATGTGGTACTGTGGGGG
GAACCGAAAACAGACACATCTGTGGGTGAATGAGTGCAGGTTGAGCTGGGACGGCTGGAACGTGCAGACCAGCAGGATGGAGCCAGGCCGGAGG
GCAGGGACGGCGTCCTGCGAGGGTGACCCGCAGGCTGGGAGCCCGTGTGATGGGGGAAGCTGTGTGTGGGGTGCCCCATCCAGGTGGAGCCCGG
GGGTGGGTGCAGAGGTCCTGGGGCTGCAGGAGTGGGGAGGAGTCCCGCACGGTGAGGGAGAGCGTGTAGGAGATGGCGGCGGGGGGCCACGTTG
GGGATTTTAATAAGTGATTCAAAGCCTGTAAGTCAAAAAGGGAAAGCTTTCTCTCCACGTTGACTTTTCAAGCACTGGGAGGAATGTACGCACC
TGGGAATTCATTCCAAATCCCTGAGTTTGCCTCGAGAGCTGTAATCCAAGTTTCCCGCAGACAGGGCACAGTCAGGTGCGTTCCACAGCCGTAG
TAAACGTCCTGGCTTCGCTGGCGGCTGCGTTCACCCCACCATGATGTGGGGCAGAAAGGGTGGCCGGGAGACGCCCGCAGCCAGGACCGCGCTC
CAGCCCTGTGCTGGCTGCCGCCTCGTGGGTGGTCGGGGAGTCTGTGTTTATTCTCATTCTCTTAACTTACTGAAAAATACTGCTTTTTAGTGCA
TTGAGAATGTATACATGGAATGTTTTACTGGAGTCTAGTTTTGAATGAAATTGTTAAGAGGTTATATGAAACCATAGAAAAAATGGGTTTTACA
CACAAAAAATGAGGGCTTTTAAATATAGTGGAAATAATAGAATAATTTATTCTAATTATGATTTATTATATATTTTATATAATTATAAATATAT
AATATTTAACCTAATAATATATAATATATAATTATTGTAAATGATAATAATAAATTGAGCTTTTTGGGAACTACTCCAAGATAAGTGAGAACAG
CAGGTGCCTCTGGCCCCTCCCCTGGGCCGCTCTCTGCCGCTCTGGTGACATCCCCGTATACGTGGCCAGCGTGGGCCCTGGAGGCAGCCAGGAC
TTGGGACAGCTCCGACTGGACCCGCTTGGTCAGGGGTGGAGGGTGGAGGCTGGAGGCTGGAGGAAGGTGGGAAGGGGAAGGTGGAAGTGGCTTT
TCTCGGGGCTGCCTCTGCTTGCCCCCTCGCCTGCGCCAATGTCAGATTCTCTCCTGCACTAGGCAAGGGCCTCACGTCGCTGGCTTCAAGGTCT
TGTTTTCCTTGGGGAAGGGGAGAGTGGCCAGCTGCCTGGCACGAGCTCGTGGGGCTGGGATGAGGGCCAGAGGGTCTGGGGGGCCAGGCCTTTA
TGCTCCCGCGTCAGCCATTGAAAGAAAAGCTGTGGGCTTTTGTTTTGGGCAGCATTGACTGTGCCGGAATCCTGAAACTCAGAAACTCCGACAT
TGAACTTCGGAAAGGAGAGACGGACATCGGGAGGAAGAACACACGGGTACGGCTGGTGTTCCGCGTTCACGTCCCGCAACCCAGCGGCCGCACG
CTGTCCCTGCAGGTGGCCTCCAACCCCATCGAATGCTGTAAGTGGACGTCCACGCGCCCACAGGGGAGGAAACCGTCCCGTCACATGCGCTTCA
CTGTCTCACCCTCTCTCAGCTTTTCGTTCGTGTAGACTTGGGACTGAACGGTTCCCACCAAACCCACCACAGTGTGTGGCCACGAGGGGTCTGA
GTTGATCATGGCTGAGTTATTGCCTGTGGCCTCCCGGGGGCCCCTGGCGCTGGCTCCTCTGATGGCTGAGCATGGCCAGGGAAAGCGGTGTGGG
CCGGGGCTTCCCCTCACGCTGCGCTGACCTTTGCGTATTTGTGCGATGACTGTGATTTGAGTGAAATTGGCTGAGTCTTAGGAAGATATTTTGA
TGACTTCTTCAGAACGAGGTGGAGACCATCAGGATGAAGTGTGCACGGCTTCTCCCCTAGAAGTAGTGGCATCCGCAGGGGTCGGCCCGCAGGT
CGTGGCGGTGCTTGCTCTGGGTGGGTCGGCTCACGTGTGACCTGCTGGGTGCCACACGTGTGCCCCAGGCCGCCCACTCAGGACAGGCCCTCAC
TGCCCCTCTCCTTCTGATGCAGCCCAGCGCTCAGCTCAGGAGCTGCCTCTGGTGGAGAAGCAGAGCACGGACAGCTATCCGGTCGTGGGCGGGA
AGAAGATGGTCCTGTCTGGCCACAACTTCCTGCAGGACTCCAAGGTCATTTTCGTGGAGAAAGCCCCAGGTATGCTCTTCACCAGGGGCCATCT
GCGGCCTGGGCTTCGGCGCTGGTGGGAACCGGTTCCCAGTCGGTGGAGCAGGACCCACCTGTGCACGGTCACCGGGACGGGGCTTATGGGGTGT
GGCACGGAGCAGAGGCTCTGCGTGGCCCGTGTCTGCATCCGGCTGTGGCTTTTTGTGTGAGCCGACAGCTGTGCTGCTGAAGGGAAGAGGTGGA
TGTTTATTTCTCGGGCCGCCGGGGCCACTGCACTCACATCGCTGCTGATACTCAGGGAGCCCACGGGCTGGGGACGCAGAGGAGGGCAAGACGC
ATTTGCTGGCTTTGCTGGGGTCCTCCATCTGCAGAGAGTGGACGCTTCGGGGGTCAGTTAGCAGCAGGGCAGGAGGGAGCTCCCCTCCGGGGCC
TTCCCCTCCCGCCAGCATTCCTGGGGTCGGAGGCAGGGATGGGGTGGGTGCCGCAGACGGCGGGAGCTGGGGTGGTCTTTAATGCCACAGCCGT
GGTGTGTGTGAGGCTCTCGTGCCCCAAGGGCACAGGTCACCCCTGCCTCCAGGTGTTGCGCTGGCCCAGCCTGGAGGCTCCGGGGCTGAACTTG
GAAGGGCAGCAGGGGTGTCCTCGGAGTGATGTTCCTCCTGGGGCCGTCACGCCTGTGGTTCTGTGCTGCCCACGGGAGACTGTCCTGCGGGGAT
TGGGCTCACAGAGTGGCTCCCAGCCCAGGTCAGTTCCATGCAGACGCCGGGCCCCAGCACTCTCATCCCAGAGGGGTGGCCTCGCCGGCTCCGA
GCAGAGTGTGTGAAGACAGTGGGCTGCACTGATGGGAAACTGGCATTGCAGGGAGAGTGGGCACCAGTGTAGCTGGGCACAGGGACCCTAGCCC
AGCCCTCAAGGGGGTTCAGGCAGAGTGGGCCTGGCTCTCCGCAAAGCCTGCCGTGAGCTCCCTGGTTTTGGGGGTTCCTGGTGCCTTGCTGGGC
CTCTGCCCAGGGTGCGCCAGGTGAGGGGCATGCCCCCACAAGGCCTGTTTTGTGATATTTTTTTCCTTTTGTACACTTACCAATTGGAATTCTT
TTGCACTTACTCCAATCCCTGGAAACCGCGGAGGCATTCAGCCTTGATAATGGGGTCTTGATTTGTACAGTTTGCCGGAAACTTAAGTGCTTCA
ATCAAAATCGGCTTTAGAGGTCTTTGACTCTAACTTGGCTTCCTATAGAATCAGGGCTTAATTGGAAAACATGTTTTAAATTACAGAGAAAACC
AACTAAGGGCAGTGACCGGGAAGGCATTTGCATTGCCCGTGCCTTCTCGGGAGGAGGCTAATTTTGAAATAATTTTCCCTTCAGAGCTTTCGGT
GTGTGTCCACGCGTTGGCGGAGAAACACTTCGCCCACAGTGTAGGGCCTCGCTTGGGCCAAGACTGGCCAGAACCGTGGCTCCCAGCTTCTGCT
TTTCAAAGCCCAGACCTGAGCAGCCTCCTTGCTTTTTCTTCTGCCCAGAGAGGCCTGGGCACCTTGGCAGAGCCTCCCCCCGAATGCAGTCCGC
AGAGGTGCCCCAGCTCCTGCCCTAGCCAAGTTACTGTGCTGAGTTGGTGCCACGGCGCCATGGGTAAGGGCAGCCACCAGCCTGCGAGGGCCAG
GAGCCAAGGGGATGCAGGACCAGCCCTGGCCTCATGCGGCCGCCCCTACAGGCAGCCCCTCCTGCCTTCATCATCACTGTGAGCTGTGGGGTCA
GCAGGCCGGAGGGGTACCTGTTCTGCCCTTAAGCTCGGCTGCCACAGCCACACTTCTGGGCAATGCCAGTTTGTCTGCCCATGTCCTCCTCTTG
TCCCATGAGGTCAGGGTGGGTCTCTGACCCTCTGCCAGCACCAGCCGGCGTCTGGTATACAGCAGGTGAGCCATAAATGAATAGGTGCTGTGCT
CGAGAGCACGTGAAGCTGTGAATACTGCAGGGATGAAGCCAGACCACGTTGATAATTTTATTCTTTATAGTTGAATAAGATAATTTGCATATTC
ATACACACACAAGCACATTCCATGTTTTCAAAAGTGGAGAACAAATGTCCACATAATTTTCTTCCTCCACTTCCGGCTTCTCAGGCGGGAGCCG
TTGCTTACAACACGTGCTGATGTGCAGTGACAGGTTGGTGCAGCAGAAAGCCCCACATCCTCATCCTGGCAACGAGAGAAACCACTAAACTGAA
TTTAAATTAAAAACACAGTTGCCCTCTCGTTTTCTGATGGGATCGTCTGGTCTGTTTAAAGTTCTGTTTTAGACTGCTCTTGCAGATGGAACCA
GGGCCGGGTGTGAGCTCAGGAGGGGAGGTTCCTGCTGAGGAAATCCGTCAGTAGGGCAGTTGAAGTGGCTTCTCCAGGAAGGGCCCCTGCGTCT
CTCGACTGTCCTGCTCATTCCGAGGGTGCAGATGGTTGTAGCTGTTGGGAATGTTGGCTCTGGACACAGGCCAGGGTCGGCTCCACCCTGTCCT
TCTCTGGAGTACGTAGGAAGCTCATTGTCCAGGCCAGCTCAGCCTCTGCGTATCAGCCAGGCCACCTTCAGCTGCTATGGGTTCACAGCATCTC
TGAGCTTTGGGAAGTTTTAGATGGAGCGGCTCCACTGCCCTGGAAGGCTGGGAGAAGCTGGTGTTGGCCTCATGAGGAAGGGAGTCCCCGCAGC
AGGGCTGCATTGCGGGGAGCCGGCCGGGCAAGGGCATGGCTCGCCGTAGCCTGGGTGCCGGGTCCCTCTGGGATGCTCCAGCAGGGCAGGAGGA
GGGTGGGTGTTTGAGTCTCAGCCTGCGCTCCACGGAGCTTCCTCCCAGGTGTCTCAGGGCGGCGCAAAAGGCCGGGAGCGGAGTCAGCCTTTCT
CTGAGGACTTAGTTTACAGCTTCCTCGGAAAACGACATTTTCCGATGATGAAAATCTGTGAGGAATGTTCTTCCAGGAGGCAGGTTGGCTGTGG
GGAGCCGAGGGCCGGGTGTTGGTTTAGAAGCCCCTGGTAGCATCACAAGAACCCCCAGTTTCCAAGGCTGCAGGGGTTCAGCTGCAGGAAGTGG
GAACCACACCCCTCTCCCACCCACACCCCTCCCCACCTTCACACCTGCACCCCTCCCGCCTGTTAAAGATCACTGTGTCTGTGTTCCGATGTGA
GGCACCTCTCTGTACACGCGTGAACGTTGAAGCTGCTGAGGGACACACTAACGTTCAACATAAACGAAACGCCCGAAGAACGCCCGCGTGAAAC
GTGAGCTCGGGCTGTGTCTAGAGCCTGGCTGAGATGAGACAGAAGGCGCAAGGTTCTGAGATGTGGATGCATCAAACATGAGCCAGTTCTCGAT
GCTGGAGCTGAACAACCTCCTTTTCTTAAGTCACGCGTCTGTTCTGGTGCCGTTTTTTAAAAACATCAGCGAAGCCTGTTTTGGTGTCGCTGCG
CGGAAGCCACACTACGACGCCGGGGGTGAAGACTCCCACTCCGTTTGCCAGCGATCGCGCAGCGGGCAGGGCTCAGCGGGTGGGGCTGCACGGG
TCTCGTCAGTGGTCGTTTCTGTGGCCGCCGTGACTGGCAGGCCAGCTGGGGCCTGGACGGCCGGCGTCTCCTTCTCCAGGTAATCAGGGCCCCT
CTACGTGGCCTCCCGGGGCCCCTCTGGGGAGGCAGCCTGGCATCTCACAGGCGGCCGGGGCTCCCGGGAAGCAGAGACGGGCTGTGCCAGGGCT
CCTAGGCCCAGGCCTGGACCCAGTGCCGTGCCAGCCACCACCCCAGGGGCCCTGCCCTGGTTCGCAGGGAGACACACAGAAATGTGGGAGGCCG
GGTGGCCTGCACGGTGGGAGCGAGTAGACAGAGCCCCTCCCGGAGGCCCCCAGCCCAGCACATCCAGAGCCCTCCCCTGAGAAACCCCAGACAG
CCAGGTTCGCCCCAAACCCAACATGGTGGGACCCCAGCTCGCCCCCCATCTCATGGCCGCCCCATCCCACGGCCGCCCCATCCTCTGGCCCTGG
ATGTTACCACGTGGAGAAGACAGGCCTGTGGGCAGGCACTAGCCACACTGCAGGAGACGCAGGCTAGGTTTCCAGATTTCTCCACACAGCAGCA
GGCGGAGGGCCTGCCTCCCACCTCACTGGGAGGGCCAGGACTCCAAATCTGGGCCAAATGTATGTTGTTATTTTTCAAATAAAGGGACCAAAGG
CCCGGGCCTTTCCAAGGCTGCCCTGCCATGGGACCGTGACCGGTGTGGTTCCCAGGACTTCCACAAGCTCTGACAGGCACCTGGCCGTTGTTTT
GGGAGGCTCCACCCAGCAGCCACAGAGCTTAGCTGGCAGCTGGCAGGACACTTGCAAGGACACGGAACTGTCACGGAACCACCGTGGCTTTGGG
CTGTCACGGAACCACCGTGGCCTTGGCAGCTGCTGGTCCTATGCCGGGACACACACATTCACCGCGGCGAGTGTGGGGTGCATGTCCCCAGGCA
GAGGCCATGTCATCCAGGTGCAGGAAGCGCCAGGCAAGGACCCTCCTGTCCGCCCAGAGAGCCGGGTCAGAGGAGTCCCCAGGCCTGAAGCCAC
TCCCTCCGGGCTCACCTTCAGGTTCCTTCAAGGCCAGGGGGAGGTTCACCCACCCCAGGGTGTCGCGGAAGCCCCGAGGCCCCTGCAGGCCCCG
ATTTCCCCAGTCAGGCATCAGTGCCCGCTGGCCACCCTGCCCCTCTCCTCCTCCAGCTGCCCCTGCTCCTTGTTTTCCTAGGGTCTGGTTCTTT
CTCGCGGGCGGGGAGGAGGTGAGGGTCAGGGCCCCTGTAAGGCAGACTAGTGGCACTGCCCCTTTCGGCCCTTGCTGTCCCTCCTCTGTCCCTT
GTGGAAGGTCTCGGCCTGGGGCAGAGGAGCGGGCCGAGGAGGAAGGTGGAGTCCAGGTGGGTGGTGGGGTCTGTGTTCTGTTTTAGTCGTGCGG
GATGTAGGCCGAGGGAGATGGACCCGGGGGATGGCTGCCTCTGTGGCATCTCCGTGGTGTGCCCTCTGACGGGGCTGGGTGAGCAAGGAGGACA
CAGCTGGGAATTCCCAGCTCCAGCGTCCGCGGCACGGGCAGCCTCCCTGTGTGGAGCAGCTCAGCTGTCACCTCCTGAAGATGTTGGATGCTGG
CCCCGAAGGCAGCATCGCAATGACAACACCTTCAGAGGTGTCCATCCCAACAGTTTAAAGGCCAAATGCAGATCTTTCAGATTTGTGTTTTCAG
GGAGAAGGTGACATTGCTGGGCGCCTGTGAGTGTGGACAGACATCACTGTCGCCGTGTGCTCCGAGAGAGGAGAGTTGGGGCTGCTTTGTCACC
ACATGGCGTCTTTGGGAGCAGGACAGGTGGCCGGGCTGAACCCCCAGGCTTCTCTGTTTGCTCCCAGAGACCACACAGGGAAGGCAAGGAGGGC
AGCGCCCGGGGAAGAGACACCTGCCCGTGTCTTCCTCATGCCCTGAGGATGGCTCAGTGGCCCAGATGGTCCAGGACCCCTAGAATTGAGTGAT
GACCCCACAGTTCTCATCAAGCCGCATTCTGGATAGCGCTAGGGGCGACTCTCAGACTCCCCCTGCCTGGGCCCCGCTGGTGTGGGGAGGCCAC
GCGTCACGTCAGTCCCCACTGCAGGAGGAGGGGCCGGGCTTCTGGCTCCCCTCCCTCCTCTCCCTTCTCAGGCCTTTTGGGATCATACACGACA
AAGTCACCCATGGTCATGTTGGCTAGTTTCTTTTTTATATCAGCTTTATCGAAATATGATCTATACACCATTTAATTCACTCAAAGTGTATAAT
TCGGTGGGTTTTGGTGGCTTCACATAATTATGCGGCCACCACCAATTTCAGAACATTTTCTTGTCCCCAGAAAGAAACCCTTGCCCATGAGTGG
CCACCCCTGCTCTCCCTTCCCCCACCCCGGCTCCGACAACCCCAATGCCCCCTCGCCGTGGCCTCGCCTGGTCTGGCTGCTCCCTGCTGGCCTG
CTGGGGAACCCCATCCTGCCGGCTGTGGTGCCTGGCAGCTTCTGTCGAGCGTGGCATCCTCCATGTTCCCTCGTTCGTGTGGGTCAGGGCGTAG
GGTCCTCAGGTCCCCTCGTTGGCACAGGTCGGGACGTGGGGTCTTTCTGTGGCTGAGGGAAGCCCACCGTGTGGCTGCCCACACCTGTCTGTCG
ATGGCACTAGGCCACGTCCACCCTGTGGAGATGATCGTGGAAAAGCCGCCATGAACCTCAGTGCGTGAGTCCTGTGTGGACGCCTGCCTGTCTC
CTGAGCACATGCCTCGGTGTGAAAGGGCTGTGGGTCACAGGGTGGCTCCGTGTGAGGCCCTGCAGGCAGATGTGCAGCAGGGCTGGGTGGCTGT
TTCCCCTCCAGAATCTCCTCTGGGAGCCAGGCTGGGACAGACTCCTGGGGGAGAGCCGGGGATGGATGAGACGCCCCAGGGACGCTCGCTTCTG
GGCATGAGGATGCTGTGGGGTGCACGGCACGAGGACGCCGCGGGGAGCAGGGCACGAGGATGCTTTGGGGAGCAGGGCAGGAGACGCCGTGGGG
AGCAGGGCTCGAGGGCACCGTGGGACCCAGGGACGCTGCTGCTTCCACCAGCGGCCCTCGTGGCTTTCTTTGTGGAAACAGTGGCTCAGCATCA
GCACCGGCGTGGGGGTGGCCCGGCTCCGGGCGGCGGGTCCTGGCCGGTCTGGGAGGCTCCTGGGCCCGCAGGCCTGCCTGTGTGGAAACCTCGC
GGGTGTGGCAGGGAAGCCTCCCGATGACTCAGACTGCAGGGAATTCCCTAATGATCTGGAATATTTTTACTCCTGCACTCGGGCAGTTTCCTAC
TAAGGACTCGGAGCTCTGATAAGGCTGAACACAAAGGCCCTTTCAGGACCGGCTGCCTTCCTTCCTGCCAGGGGCTGAGTTTTGAACAGCACGG
GGGCCGGCGCTGCCCGTCCGGGCCTCCACGGAGCTGGGAGCCGCCATGGCGATGCTCTTGCCGGCGGGACGCGGCCCTGCGTCTCCTTTCCTGA
GATGCGAGGTTATTTCCATCTTTCAGCAGTCTCGGCGCTTTGTCTTGTTCTGGAAGCAATTGCTAGATAGACATTTCTGGCCGTGAATACTATT
GCTTTTGAAAGACAATTACTTTATGATCCTTAAGCAGAAACAGAAAGCCTGCCCTCACCCTCACCCTGGTCGACCTGAAACGGCCACAGACGTC
GGACACCACGGGGAGAGGCCCCACCTCCAAGAGGGAAGGCCACAGGCGCTGGCTCAGCCGGGGTCTTTGAGGAAGGCAGGGATGTTTGTCGGTT
CCCCGCACTTTTAGGGGAAGTCTAAGCGTATCTGCTCCCACCTGCGCAGGAATTGCAGGGTGCGGGTGCTCCCAGGTGTGCGGTGGAGGCTGCT
CCATGGCAGCTGCTGAGCTGGGCCTGGCTGGCCACGCAGACGTGGCTCCCCTTGGGGCCGCTCCGGCAAGGATGGTTCCCCACAGGCCCACGAG
GCACGGGTGGGAAAAGACGCCTCCATGCCGTCGTTCCATTTGCTTTTCATGGACTCACCGCTGCGGGGCAGCCACCTGGAGGAGGCCGCGGGGG
AGACGGCTGGAGTCCCTCTTGCTTCTCGGGACATAAAGAGAGGAGCAGGCTGTGTTGGCCTCGGCTGCAAAAATGGCGTCCCCTCGGGGTGTCA
TTAAACGCGACATCCTGGCCCCGTFTGCGCCTCAGTGTCCCTCCTGCTCAGCACAGTCACTGAACCTCCCTCCCCTTGCCAGTGCCAAAGGCCT
CCACAGCGTCAGGGAAGTTCCTAGGGAAGCTGAAGTTGTGACTCTGAGCTTCCTGGGCTGGGCGGGAAGCAAACTATAGCCTGTTGCGCCCGGG
AGCTTCAGAGGACCACAGCCTTTTGTGGGGTGTGGACGGGGCCTCACCCCAAAGATGGACTTTGTCATAAGAAAAGTTCAAAAGTCATTTTCAA
AAGGTGCCAAACAACCAGAAGACTTAGTTACACACAGAGTGATTTGGATTTTTATGAAGTGGGGGTGGAGGGGCTTCTGAGTGGTGCATCTGGG
CTGGGGGCTTCTGGGAATCCTGTGGCATCAGCACCTGTGCTGGAGTGAAAACGCCTCTTTCTTCCCAGCTCCTGAGCTTTCCAGGGCCTGGAGG
ACTTGCTGCTTCATGAAACTCAGATGGCTTCAAGCGCAACTGAGTGTACTTTTTAGTTTCCTGTAATTTTTTTCCTAGAGGAGAGAGGGATGCC
AGGCTCTATCGAGTGGGTTCATCTGGTGGGTAATTTGCGTGGGGACCTCGGGCCTCCCGGGCTGGCATGGCCAGGGCTTGGCTGGGCGGCTCTG
GGGTCCTGTGTGACTCACATTTCCTCTCCACCCTCCACCCAGGCAGCAGCCCGGCACTGTAACAGAGAAAAAGGAGATTTTGTCTGATTTATCA
GTAATTGAGGGTTTGTAACAGACACACATTTTCTTCTTTGGAGACAGAGCATAAGGATAAACTTAAGTGTGTATACCTGTAACTGTTCATTTTG
GGAACAGACTTTATTTTGGAGAAGATAAAAGGCAGGAAGAGACTCTGTAGGAGATTGACATCTGTTAATTTGAGATAATTGCCCCAAACTCAGC
CCATCTGAGGGGCCAGGCCGTGCACACACCCAGGCTGTGGTGGGGCCGCCTTGTCCGTGTGCGCCTGGAGCCCCCACTCTGCCAGGCCTGGAGC
AGAGAGCACAACGCGTCCTTTCACTACACCTCTGGGAGCCTTTTTATTTTTTAAAAACATGCAAATGGCCACTGGGGAAATGCCAAAAATACCT
CGTGTTTTGCATTTCTGTCTCCTGATGGGCAAGAAGTTGTCAGAGGAAGGCTCTTCTCATTTACCCAGTGGTTCCTTTGCACACAGGCAGGGCC
AGCGGCCAGGCCCTCATCCACCAGAGTAGACCCCAGCACGAGCAGGCGTCGCACCCTGGCGTGGAGCCCCCGGCCCCCCGTCACTACTCTCACA
CCTCCTGCATCCCTGGGAGGCTGGGCTGGGAACTGCAGAGCCGGGTCCCAGGTCCACGCACAGACGCCCTGCACACGCTCTGCTGATGGGGTAC
CTGCTCCCCCTCACACCTGCTCTCCCTGGAGAGGACGAGGGCATAGAGACCCTCCCGTGACCTGTCCTGAGGCTGTGAGAGGCCAGCAGATGCA
GGGCCAGGACGGATTCCTGAGGATGCCCCTGCCCTGTAGTCCCTCCTACGCCGGGCTGGGGTCAGAACAAGCCCTGAGCCGCGTGTCCTCAGCT
TCTCCAGGCTCCCAGGGCAACTTTGGTGTCGAGCTCGGGGCCCTGCCAGGGCACGTGGCTCCTGAGGTGCTGCCCTCGACAAAGCCCTGGGATG
GGCCTACGTGGGTCTCCTGGGACAAGGCGCCCTCCTGGCTCAGGGCCCTGGGTCTGGATCACGTGGGGGTGTCTGGGGAGGGGGCTCCCCACCA
CACAGAGTCACAGACGTTTCCTGCTCCTTTCTTCCAGATGGCCACCATGTCTGGGAGATGGAAGCGAAAACTGACCGGGACCTGTGCAAGCCGG
TGAGTGCCTTTGGCGCAGCTGGAGCTACTGTGGGTCCCCAGGGCTTGGGAGGCTGGGGTCTGCTGGAGCCACTGCGGGTCCCCAGGCCTTGGGA
GGCTGGGGTTCCTGTTTCTTAGAATGAAACAAATAAAAATAGTGCATAGAGTAACAGAACCAGTAACAAACAGGATCACCAAGTACTCGCCAAA
GCACCATGTGCTGTCACCCAACGTGTCAGGTCACGTCAGAGCCCTGGCGGCCGGGGACGGTGCTGGACAGGCTGAGGGCCACCTCACACACCCT
GGCGCTGAGACAGCCCAGGGAAGGTCGTCTCACACGGACCCTCCCGCCCTTTGCTGTCCCCTTGCTGTCACCATCACCTGGCACAGAGCAGGCT
CCTGGGAGTCAGTGCTGTGCTGAGTTCCCGGGGGGTGGTCAGCACCTGCCCAGACCCGCTCACAGCACCGAGGGCAGGGCTCTGCGGCATCCCC
AGTGGGGACTCCTCACCCCGTCCTCCGAGGCCACTGATTGCGCCGACCTCCCCTGTGATGGTGTCCCAAGCACAAGAGCCTGGGCGTGGCTCCT
GGACTCGCGATTGGCCACAGCCTGTTAGCGGGCGGTGCATGTGTGCGGGGTCGTGGCAGACGCGTGGCTGGGTCACACTTCTGCTGTCACCGGG
CCGTTTCCAACTCCCCCAGTCACCTGCCACAGCTTCCCTGGCACCGCGTGCATACAGCCGGTGCCTGAGGATAATGACCGTCCTCGGATTGGAG
ATGACCTTTTGTGTGGATGGCTGTAGCTGCTCCCACATCTCCTGCACGGCACCGGTTGCTGCGTGCATCTCTCCCATCCCTGCGCTTCTGATCC
ATGTGCTCATGTTTAAAGTGGTTTCTTGGAGGCAACGTCTTCTTGGGTGGTGTCTTGGATCCTCTCTGACGCTGTCTTGTAATTGGCACATGGA
GACCATTGATGTTTAGGTCTTTAGTTTTGTTTGTTTTTGTTTTTTTGAAACAGAGTTTCGTTCTTGTTGCCCAGGCTGGAGTGCAGTGGCGTGA
TCTCGGCTCACTGCAACCTCCACCTCCCGAGTAGCTGGGATTACAGGCATGCACCACCACGCCCGCCTAATTTTGTAATTTTAGTAGAGATGGG
GTTTTGCCATGTTGGCCAAGCAGGTCTGGAACTCCCAACCTCAGGTGATCCGCCCGCCTCGGCCTCCCAAAGTGCTGGGATTACAGGCATGAGC
CACCGTGCCCGGCCTGATGTTCACTTATTATTGATACAGTTGGATTGATCCCTACGTTGGATTTTTTGAGTGTTCTGTTTAGTCCTATAAAAGC
AGTAGAAATCTCTAGGCCAACATAAGATGCCACATGGCTCACAAGGGAGTTTTGAAGACATGAACATGCTTCTGAGCTCAGCAGTGTGTGTCCC
TGCGGGTCAGGGAAGTCTTGTGCACCCACTCAGTGTGTGTCCCTGCGGGTCGGGGAGTCTTGTGCACCCACTCAGTGTGTGCAGTTGGAAGCTG
GTCCCTTCAGAAACCTCAGGGAAGAGCCCATCTCGGGCGGCTGGAGGCAGGTGGACAGGACACCGGGGAGGAGTCTTGAGGTCGTCTCCTGTTG
GTGGGACGATCGGTGGCCTCCATGGAGAGCGGTGCTGGCACCTTCACTCCCTGTCCCAAGAGGTGGCTCTGTCAGAAAGCAGCGTCTTCCACCC
TGCGACTTCAGCGCCCTTCCCCCAAGGACCCATCCCCTTCCCAGAACCTTTTCCAAGTGGTTATCAGATGGAAAAGGTTCCTCCCTGGCCCTGG
GTGAAGTGAGCTCACCTGCACCTTCAGTGGTGCTCAGGCCTCCCCTGAAGCTCCTGGCACTCACAGGGTTCCAGTTCAATGCTGTCGCTTTTCT
TGCCTTCACCTGAGAAGCCCCTTGAGGAAAGCTGGAGGCCCCAGGGCAGGCCACTGTTCAGAGGAAGGAGTCCCGGCCTACACGGCCCGTTGTC
CGCAGAGCCAGTGAGTCAGGGTGCTCAGAAGGGTCCTGGGAACGGCCAGCCACAGGCAAAGGCAGCTGGAGCTGCTGTACCGGCCTCTCCTCCC
CGCAGCCTGTTCCCGTGTCCATACCGGCCTCTCCTCCCCACAGCCCGTTCCCGTGTCCATACCGGCCTCTCTTCCCCACGGCCATTTCCTGTGC
CCATAAGGTCTGTGGCTCCCCGCTGGCCGGGCGTTTGCAACTGCCCAGGGGGCTGGCAGCCTTCGTCCATGGGGTCATTGCCAGGCCTCCCCAC
CTACTGGGTGTCTGATTCGTTCCTCCCCACTGGGCTGCCCTTGCTCTGCTGTGCGTCTGTGCTGGGTACACTGACATCTGGCTCTCCGTCCCTC
TGCCTGCAGGCAGTGTACCCTGGTGCCTGGCCTCGTGCTGCCTACCCTCCCTAGCCCTGGCTCCGTCGGGGTCTCCACAGGCTTCGCCAGCCCC
GCACATGCACGCGGGTTGCGGGATGCAGGATGCCGGAGCTGGGCTGCCCAGCCGCTGGCCTGCCCTGTCCCCCTAGGCAGGGAGAGCAGCCAGG
AGGAGCCGTGAGCCTTTCTGCACTGAAAACAATCCCAGCCAAGCTGACATCCACATTCTCTGTGATTTTCTGTTTTACTTTTGACACTGGCATA
TTTCGAGGGAAGCTTAACAGCTGTATGTTTTAAAAGAAACTTAAGAGCACATGTGAAAAATTGTGTACTTCAAGTTTACTGGTTGTTTTTTGTT
TGTTTTTTGTTTTTTTGAGACAGTCTCGCTCTGTCACCCAGATTGGAGTGCAGTGGCTCGATCTCAGCTCATTGGAGCCTCCGCCTCCCAGGTT
CAAGCGATTCTCATGCCTCAGTCTCCCGAGTATCTAGGACTACAGGCGCCGGCCACTATGCCTGGCTAATGTTTTTGTATTTTTTTTAGTAGAG
ACGGGATTTCACCATGTTGTCCAGGCTGGTCTTGAACTCCTGACCTCAGGTGATCCGCCCACCGTGGCCTCCCAAAGTGCTGGGATTACAGGCG
TGAGCCACCGCGCCCGGCCTGCTGTTTCATTTTTAATCTCTTAAATGGAGTTTGCTTTAATATATATATTTTCTCATCATAAAGGAACTTCATA
CTCATCGTGGACATTTGGACAATATAAAAAAGGAAATAAAACCTAGTGTAGTCTCACCAACTGAGACATAACCACTGCTGGCATTGGGCATGCG
TCCTGCTCCATTTGCTGCTGTGTGTGGATGTTCACGCAGACACACACACACACACAGAGTACATTATATTGTGGATATATGTGTTTGTGTGTGT
GTGTGTGTGTGTGTATATGTATGTGTATATATATATATTTATTTATTTATTTATTTTTTTTTTTTTGAGACAGGGTCTTGCTGTGTCATCCAGG
CTGGAGTCCAGTGGCTTGATCATGGCTCACTGCAGCCTTGACCTCCCAGGCTGAAGCAGTTCTCATGCCTCAGCCTCGCCAGTAGCTGGGGACA
CAAGTGTGCCCTATTCTGCCCAGCTAATTTTTAAATTTTTGTAGAGATGGGGCCTCGCGGTGTTGCCCAGGCTGGTCCTGAACTCCTGGGCTCA
AGCAATCCTCCCACCTGGGCCTCCCAAAGTGCTGGGATGACAGGTGTGAGCCACCGTGCCCGCCTGTGCATATTTTGTGAACAAAATGGGATGT
GCTGTGTCTACGGTTTTTGTACTTTCTATAAGTAAGTGTATGAGCTTCATCTCAACAGTGGAGTTTAAAGCAGGTTGCGTAGGTGCTGGTGCCA
TTTGGAACCCGTATTTTTTCCGGTACAGACGAGTTTCCCTCTCCCTTCCTGTTCTTCCGTGTTTCTTCTTTATCGCTTCCTTCTGTCTGTATTT
AAGTGGCAGTCTCCCACGGGAGATAGTATTTTTGTCATATAATTTGTCTATTTAGAGAGTTGAAGTGGGTTGGCCTGTGCCCACATCAACATAG
GCCCTTCCAGTCAGGTAGGACTTAGGAAAACTGGCATGCACCGTCCAGCATGCCCCACAGGGTCCCACCTCCACCCATCCAGCCTGCCTCACGG
GGTCCCCGCCTCCACCCAGTCTGGCCCACAAGGTCCCGCCTCCGCCCAGCCAGCCTGGCCCACGGAATCTCCGCCTCTGCCCAGCCAGCCTGGC
CCATGTGGTCCCCGCCTCCACCCATCCAGCCTGTCCCACAGGGTCCCCGCCTCCACCCCAGCCTGTCCCGTGGGGTCCCCGCCTCCCCACTCCT
CGCTGCTGCCTGTGAGTGCCTCCCGGCCCGTTACTATTTCAGTCTCGGCTTCCATGAGACGTTTCCTTCTTGTCTCTTCCCTCAGCTGCTTCTG
CTCTAAAGACCCACCTGGTGTAGCTGCTGACTCCATCGCAGCTGCCCTGAGCACTGCTGAATTCAGAAAACAGCCTTGGAGGAAGCGTCTCTTT
ATCCCCCGTACAAATGCCGAGACAGGCTCCGGGTGGCCAGAGCTTCCCGGGTCAGCCCAGGAGATGGCCCAGACCCACAGTGCTCTGGGGGCAG
CCCAGGCCCCGCCCACTGACAGCACTCATGGCCCCTCCGGCGGCAGCTTCAGCTCCCCTGGGGCAGCTCTGATGCGGGGCAAAGGCAGGAAGAC
GCCCATGTGCCATGGCTTCTGCTTCTTCCCAATGAACTGCAGTTAATTAAACAACACAGACCTGCTGCCAGGAGAAAGTGTGGCCCCTGGGCAC
AGGGCCCAGTGGGGTGGGGAGCAGGGGTGCGGGGAGGGCATGGCCCCTCTGCATGGGTGTCTGGGCCTCAGCTGTTTCATTATTTGTAAATGAA
AGAGCCTGGACCTCAAGTTGTCTTCAGTTTTCTGTCTTTAACACTCTTAAGATTTCATTGTTAATGTGATTCAATCAATTTGGCTAAGTTAGGT
GCTGAGCTATAATTAATTATTTTAACAAAAGAAGTAATACAATGGATTTTTCTCCTGCTTGTTAAACTAAGTCATATGATTATAAAAATGAAAC
TTTATAGATGCGACTCCCGCCAACCCTCTCCCCAGATACCAAGTGCCGCCCTCGGCAGGGATGCCTGCATCACCCCAGTCATTTAGGGTCATCA
GGGGCCCGAGTAACGCTGGGGCCTGAGCACGCGGTTGGGGTCTCCGTGTCCCAGCCCCCCACCCAGCCCCGACCTCTGCGAACAGCGCCACACG
TCCCATGGCTTGTGTTTCATGTGTATACTTGAAGGAGCATGAGTTTCGTAAACATGCTGCTGGCTGTTACATTAAAAACATGAGAAGGTAACGG
CAGCCTCCACACGCCGCCCTCTGCTGTCTGTGCCTCCGTGCCCTTGGCTGTTCCCACCCCGGGGTCTGCGGGGGTCAGGGCCTGCCTCCTCTTC
CTCAGTTGGAGTGAATTTGGGGTCTGACTGAGGGCCTCGGCCATCGTCAAATGTGACAAACACAGGCCCTCCCCAGAGACCGCGTCTCCAGGAA
GCACCTGCTCCTCTGGGTTTCGCTGCAGTCACCTGAGCAGGTGCTGGGCCACCGAGCACTTCCATCACACAGTGGCTGGCGGCGGAGCCTTGGG
TCGTTTTCTGAAGGTCACCAACCCTTTGGCATCAGAGCAGGCAGAGCCTTTTGCATCCCCGCCAGTCCCCTTGCCCAGGTGGCCCCGGGCAACC
TCCACCCTCAGGCACCCGGTCCCAGTTCTGTTTACAGCTTCTCTTTTCCTGGCTTCTTTCCCCATATTGACAAGATTTCAGTCTTAGGTTGGTC
TTGTCTGCTGTGCTGCTCTGAGGAAGCCTCTCGGCTCCTGGGTGTGCAAGAAGCCAAGGGCAGCCCCTTCCCTGTGAGCGTGCGTGTCCACACG
CGTGTGCAGGTGGCACAGAGCATCCAGGGCTGTCATCAGCTGCTTCTGGACCGCCCAGGTGGCTGCTTGGGAGTTTACCGTCACGGCAGTCCTG
TGCTGCCGTGGAAACGCGGGGTTGCCGTGTGGCCGCCGTGGAAACGCGGGGTTGCCGTGTGGCCGCCGTGGAAACGCGGGGTTGCCGTGTGGCC
GCCGTGGCGCGGCAGCCATCGCCTGCCCGGTGCTGATTGTGCCTCCTGTGTGCCCTTCTCCTGTAGAATTCTCTGGTGGTTGAGATCCCGCCAT
TTCGGAATCAGAGGATAACCAGCCCCGTTCACGTCAGTTTCTACGTCTGCAACGGGAAGAGAAAGCGAAGCCAGTACCAGCGTTTCACCTACCT
TCCCGCCAACGGTAACGCCATCTTTCTAACCGTAAGCCGTGAACATGAGCGCGTGGGGTGCTTTTTCTAAAGACGCAGAAACGACGTCGCCGTA
AAGCAGCGTGGCGTGTTGCACATTTAACTGTGTGATGTCCCGTTAGTGAGACCGAGCCATCGATGCCCTGAAAAGGAAAGGAAAAGGGAAGCTT
CGGATGCATTTTCCTTGATCCCTGTTGGGGGTGGGGGGCGGGGGTTGCATACTCAGATAGTCACGGTTATTTTGCTTCTTGCGAATGTATAACA
GCCAAGGGGAAAACATGGCTCTTCTGCTCCAAAAAACTGAGGGGGTCCTGGTGTGCATTTGCACCCTAAAGCTGCTTACGGTGAAAAGGCAAAT
AGGTATAGCTATTTTGCAGGCACCTTTAGGAATAAACTTTGCTTTTAAGCCTGTAGTCCTGATGTGGTCTTTAAGGATGGTGAATGAGCTTTGT
GCTGGGCGGACGTCCCCGACACACTTCTCCAGGGGTAACTTCATCTCCTGGGACCACGGGCATCCAGGCTGGGGCACCTTCTCCTCAACCTGCC
TGCCCCTCACCCGCCGTGGGAATGGCTCCTTTTCACTCATACAAAAAAAAAAAAAAATCATCTTACCTTTTTAACCTTTAGGGTGTTTTCTTAA
AGAAAATACCATCCAGTTTGTTTCTTCAGTCCCTGAACCAGTAGAAGAGAAAGGCGTCCCCCACGCCATCCCCGGCTTCCTTTGTGGGTACAGG
CCACTCCCCCCAGCTCGGAGAGGACAGAATTAGCAGTCATCCTGAATTTTACTGTATTTTGTGGGTACAGGCCACTCCCCACAGCTCGGAGGGG
ACAGTTAGCGGTCATCCTGAATTTTACTGTATTTTGGGTAAGCTTAGCTAACATCAATAATCTTTTTATATTGCTTTTTTTCTAAAAACTGCAG
AGATTCCTGTCATCTGAGAGGGAAATTACACACTGGGCGCTAATTGCAAAAGCAGCTGCGTTCTCTTAGTAAAATCAGGGTGTTTTGGAAGGTT
TCCATTTTCTCTGAATGTAAACAGCATTTGTAATAGACCAGATCCTAAGGAAGCCCTTGAATCCCCACGTTCACACCAAACTGTTGGGACTCCT
TGTTTTTATCAGGCAAGCTCTGGAAAGGCCTCTCAAGATGTGGAAAATGGGGTTTTGCTGTAAAGTCGCTGTACCCTAAGAGTGTCTGTGAGAC
AGAGCTGCAGGGGTGATGTGCAGCCCCCAGCCCAGCCCTCGCGGGGAAGAGCCTCACGCTCTGTGACCCCCAGGATTGTGGGGAGGGCCTCATG
CTCTGGGACCCCCGGGATTGTGGGGAAGAGCCTCATGCTCTGGGACCCCCGGGATTGTGGGGAAGGGCCTCACGCTCTGGGACCCCCAGGATTG
TGGGGAAGAGCCTCACGCTCTGGGACCCCCGGGATTGTGGGGAAGAGCCTCACGCTCTGGGACCCCCGGGATTGTGGGGAAGGGCCTCACGCTC
TGGACCCCCCTGGGGTTTACGTGGTCTGTGCCGCCCACACTTAGTGAGTCCAGCCCATTCTTGGAACAAAGTACAATATATCTTACAGAAGAAA
ATATTTTATCTTTTTCTGAAAATATAATGATTGAGTCCTAATTAAACTTCAAGCATGGTTGACCAGAAATTGAATGTTTTAAAGAAAACCTAGG
CCTTATAGAGAAGGATTGCTTTCCAGAGCAGTGCATTAATTTTTTTCTTATTTGCTCAGCATGCGGCAGTCCCCACCGTGGGTTTGAGCAGCAC
TGACTTTAGCACAGATCACGTATCTCAGAGGCAGAAGGGGCGTGCGGGGAGCGTGCCTGCCCTTCACAGGTGGGAGGTGGCGGCCCCCAGGACC
GTGGCCACAGCATGAAGCCGGTGGGGCTGAGCCGCTCCTGCTACCTCCAGTCCACACCCCACCTGGCAGCCGGCCGTCCTGTCCTACCAGCCAC
ACTCCTGCCTCGATTGGCCCCGGCTCCCCTCTCAGCATTGTGCCCAGCGTGGACATCTCTCCTGCACCCCCTGCCCAGTGTCTCGGCTGCACCC
TCCACCCCCCATCTGCTCCAGCATGGCTCAACGCAGGCCAGGTTCCCCAGGCTCACCCTGGGCACCAGCCTTCGCCCGTTCTCCCTCTCTTTGC
CTCCGTCGTCTCTTCTCTCCTGGACGTAGCACCATAGTCCCTGGGTGACCATCCCAGGTCCCCACAAGCACACTGACCAGCCCCACCAGCCCCC
AGGGGCTCCCCGGCTCCCTGTCACCCCACCTGGTCCTGGGGGCTGCGACCGCAACCTGCACCAGACTGTGAGCCCCTCACGCAGAGCCCCCAGG
CTGCCGCAGGGCGAGATCCCCAGTGTTGACGCTGGGCCTACCTCGAGGCCACTGTCAGTGTGGTTGTTAAATAAATAATGAATAGAAACCAGCA
CCCAGGTATCCCTGTTCCGTCCCGCGTGCCCGCTGCACTTCCTGCCTCTGCAACCCCGGCTGGGTCTGAGGACGCCGAGGCCGCTCCAGGACCT
GCGTCCTCCGTGCTGTGCATCTGTGTCTTGGGGTCCAGCTCAGATGGCTTCTGCCACGTGGTCCGAGAGATAGTTGCCAGCTCTGCTTCTGCCG
ACCCTGGAGGTGGAGGCAGGACGGGTGGGTGTGGGTGGAAGACAGCTGCTTAGCAAGCACCGCCCTGACTCAAAGCTGCTCTTGGCTTTTTCCC
TTTTCTACACCGTCATCATGGGGTCGTTAGCAGTCTAGGTTTCCGCTGTGCCGATGAGCTTCGGGTCTGCATGCCCGGGGGTCACGGCAGTGAG
GGGCGGACTCCTAGGGTTGTGAAATGTGCCGTTGAAATGCACAAGGTGACAACAGTGAACCCACTTACACTCCAGCTGTGGAATTCATTTCACG
TACATGTTTGTAAAGTCAAGTCTCATTGTAGGTTTTCCTTCAAAATTGCCATCCCCACAGGCAGCACAGGGAGGTCCCCTCGAGAGCTCCAAGC
ATTTGGAATCCAGGTGCCCGAGGTGTCCGGTGCTTGTGGGGGGCGGGCTGGGGACACGCCTTGGCCACCAGCCGCCCGCACTCCTCGTCTCAGT
GCCAAAGGCATCCTCCGATGACTCGGCCGAAGATGAGCTCAGTCCGCAGCCCCTGTCCCGGCCCCGCCTCCCGTCTTCAGGGGGCCTCACGTCT
CCTTTTGAGTCAGAAGGTCGCAGTGGTCGGCCTCCGGGAATCAGGCATCTGAGTGCAGGGGGCCCCCGGGCTCAGGTGGGGCTGACACCTCTGG
CTCATGGTCTGTGATGGGGCCCAGAGGGAGAGTTCCCTTCACCCTCCCTGGGGCGGGGGGCAGGCATTAAATGGGGAGAAGTTCCCAGCAGAAT
GTATGAGCCCCATGAACGGAGTGGGCATAGCCCTCGGGGAGTCCTGCCGGCCGCCCCTCCTGACATCCCGGGGTGCTGCCCGGTGAGGAAAGTG
AAAATGTTGTCTGCAGTTAGAGGCTGGAACACACATGCCCACAGAGAGGCTTCCTGAGCGGCTTGTCTGTCACTGTTTTTGTGTAACTGCGCCT
GAATTTAATCTCCCGGAAGGCGCGTTAGGAGGGCTGAGGGATGATCTGGTGCCAGGAAGGTTGGCAGTTCCGAGAGGTTATTCCAGCACGTCAG
CATTAAAGGAGAGAGGCGAACCTGGAACGTGGGAACCCTGCCCAGGTCTGGTTAGAAAGGCGGCGTCCGCGTGGCCTGGGGCTTGTGTGAGGGG
TGCACTTGTGCCCCCAGATACAAGCAGGCAGGGCCGGGAATCTGTCTCGTGGGGGGCTGCGGGCACTTTGGAGAAAGAAAACCAAGTTACAAAC
TCAAAATTACCTACCAAAGTCAGTATTTTTTTAAACGAGAAAATAAAACATGACAAATGCCAGGTCTTTTTGTCTGAAGTCGTTTTAGTCGGTT
ACCTGGAAACGCTCCCAGGGCCCCTGAGCTGGGGACACGGAGCTCTTCCCCCATCAGTGCCCAGGAGAAGCTGCTCCCGGCCCTAGGTCCCTGT
GGGAACACACAGCCAGCCCTGCCTCTGTGGGGGTCCCTTTCGTGGCCAGAGCCCCCTTCCTCAGGGGACACGCGGTCCAGCTGAGGGTCATCCC
GTGACAAGGAGTGGCTGCTGTGATGACCCAGGCACAGAAACAGCTTTGACCACAGAACGTGACATCCAGGGTGTGAAGGTGTCGGAACAGATCT
GCCTCGGGATCTGGGGGTCCTTCACCAGGGGTGGGCGTGGGCCCCGAGCCCAGACAGGAGCCAGCTGGAGACTCAAACGTCACGAAGAAGGGAA
GACGCGCTTCCCTCCCCCGGTACTAACCAGCCCTTCTGCTGGCCCTGCAACGATGACCTAGGCTTGAGAATCTGTTTCTGCCTCAAGGGGTAGA
AAGGGGAATAGATTCCATTACTTCCCTGTTTGCAATGGATTATTAAATCCAGCAATAACTGTGGCATCGGAGAGGTGTTGGTACAGAGGGAGCC
GGGGTCCTGTGGCCTTGGGTCCACCCCAGCGATCATCACGGGAGAGAGCTGCTTAGGGTGTGAGCCGTGGGGGGGCGGGGGGCGGCTGTGAAAC
TGAGGTCCCCGTGCACAGATCCCACCACCCAGGGTCGACAGCTGGGGGCTTTCTCTGCTGGCACTTCCCAGGCAGCAGAACCGGCCCCTCTAGG
AAGCCCCCTCCAGGTGGCGTGGCCCAGGCCCTCAGTAAGCTCAGCTGGGGCTGTGTGAGCTGAGGGGCTCAGAGCACCCCACAAGGGATCCGCC
TGATTGCAGACAAGCAGTTCTGGGCCGCCGCCCGACCTCCATGCCCTTCTGCTCAGCCCGGCAGCCTCTCCTTCCCAGGGGTTTCCCATTCTGC
CTTTTCCCTGGACTTTCCAACTCACTTTGTCCAAGTCCACTGCATGTAAAGCTTGATCCTGAAACAGTGAAGTTCTTGAGGTCTCTGCAGATGC
TCAGCGGCCCCAAATCACCCTTTCGCTGCCATTGGCCAAAAGCACAGCCTGGGTGGGCCTTGAACACCTGTGGCCTCTTCCCCTCCCCCTCCCC
TCACCCCACACACTTGGAGACACCCTGGGGCTGGGCCTGAGCTCGGCTCTCACTGCAGAGTAAACCTGCCCACCTCAGACACCCAGGCATGGCT
CCCGCCTTCCCTCATCTGATCCTGCCACCGTGGCGGCTGCCTCCAAGCTCCTGACAACGTCAGGTCAGCCCTGGCCTCGCGGCGCTCTGGGTTT
CAGCCTTAAGAGACTTTCCATGTGTCGGCTGTGGGCGCCTTGACGCACCAGGGCCTGGTCACAGGTTCTCAAAGCAGAGATTCTCCTGGCACCC
CTCGTGCGCCCTCCAAGGGCCTAAAACGCAGACGACCCACACTCCATGCCATGTCCTGCACAGGAACCAGGGTGGCCTCCCCTTTCTGCAGCTT
GTGGGGGTCAAGCCGGGACCCCTACACTGATGTTTCAGCCCAGCTCTGCCGCAGCTTAGAAGTCCTACCTGATTTCAGACAGAGCGGATGATGC
TGACATTTCCAGCCCACAGCGTGGCCTGCGCCACGCTCCAGGTCGCCACAGGGGTGAGAATTCTGGGAGCCCAGCTGCCGAGTGCTTCTGGGGT
TGGAGCTGGGCTGGCCCCACCTGTGAACAGCGGCCTGTGGGCCCCTCATCCCTGTTCTACTGAAGGCCCCACAACCACCTGAGGGAGACAGCTT
TTATTAAATTTCAGTTTTTGTCGGTTGCCTCGCTCTTCAGACGGCAAGTGAGAGAAGCGTGTTCTCACGCTTACTGTCAACGTTGCGAGGGAAG
CGTGTTCTCATGCTCACTGTCAACATTGTAAACCTGAGGGAAGCGTGTTCTCAGCTCATTGTCGACGTAAACCCCAGGGAAGCGTGTTCTCACG
CTCACTATCGACGTAAACCTGAGGGAAGCGTGTTCTCGCGCTCACTGTCGACGTAAACCTGAGGGAAGCGTGTTCTCGCGCTCACTGTCGACGT
TGTAAACCTGAGGGAAGCGTGTTCTCACACTCACTGTTGACGTTGCAAGGGAAGCGTGTTCTCGCGCTCACTGTCGACGTTGTAAACCTGAGGG
AAGCGTGTTCTCACACTCACTGTCGACGTTGCAAGGGAAGCGTGTTCTCACACTCACTGTCGACGTTGTGAGGGAAGTGTATTCTCACGCTGTC
GACATAAACCTGAGGGAAGCGTGTTCTCACGCTCACTGTTGACGTAAACCTGAGGGAAGTGTGTTCTCACACTCACTGTCAACGTTGTAAACCT
GAGGGAAGCGTGTTCTCACACTCACTGTCGACGTTGTGAGGGAAGCGTGTTCTCACGCTCACTGTCGACATTGTAAACCTGAGGGAAGCGTGTT
CTCGCGCTCACTGTCGACGTTGCGAGGGAAGCGTTTTCACACTCACTGTCGACGTAAACCTGAGGGAAGCGTGTTCTCACGCTCGCTGTCAACG
TAAACCTGAGGGAAGCGTGTTCTCACGCTCACTGTCGACGTTGTAAACCTGAGGGAAGCGTGTTCTCACGCTCGCTGTCGACGTTGTAAACCTG
AGGGAAGCGTGTTCTCACGCTCGCTGTCGACGTAAACCTGAGGGAAGCGTGTTCTCATGCTCACTGTCGACGTTGTAAACCTGAGGGAAGCGTG
TTCTCGCGCTCACTGTCGACGTAAACCTGAGGGAAGCGTGTTCTCACGCTCACTGTCGACATTGTAAACCTGAGGGAAGCGTGTTCTCGCGCTC
ACTGTCGACGTTGTAAACCTGAGGGAAGCGTGTTCTCACGCTCACCGTCGACGTAAACCTGAGGGAAGCGTGTTCTCTCACTGTCAACTTTGCA
AGGGAAGCGTGTTCTCGCGCTCACTGTCGACGTTGTAAACCTGAGGGAAGCGTGTTCTCACACTCACTGTCGACGTTGCAAGGGAAGCGTGTTC
TCACACTCACTGTCGACATTGTGAGGGAAGTGTATTCTCACGCTGTCGACATAAACCTGAGGGAAGCGTGTTCTCACGCTCACTGTTGACGTAA
ACCTGAGGGAAGTGTGTTCTCACACTCACTGTCAACGTTGTAAACCTGAGGGAAGCGTGTTCTCACACTCACTGTCGACGTTGTGAGGGAAGCG
TGTTCTCACGCTCACTGTCGACATTGTAAACCTGAGGGAAGCGTGTTCTCACGCTCACTGTCGACGTTGCGAGGGAAGCGTTTTCACACTGTCG
TGTTCTCACGCTCACTGTCGACATTGTAAACCTGAGGGAAGCGTGTTCTCACGCTCACTGTCGACGTTGCGAGGGAAGCGTTTTCACACTGTCG
ACGTAAACCTGAGGGAAGCGTGTTCTCACGCTCGCTGTCAACGTAAACCTGAGGGAAGCGTGTTCTCACGCTCACTGTCGACGTTGTAAACCTG
AGGGAAGCGTGTTCTCACGCTCGCTGTCAACGTAAACCTGAGGGAAGCGTGTTCTCACGCTCACTGTCGACGTAAACCTGAGGGAAGCGTGTTC
TCACGCTCGCTGTCGACGTAAACCTGAGGGAAGCGTGTTCTCACGCTCACTGTCGACGTTGTAAACCTGAGGGAAGTGTGTTCTCACACTCACT
CGACGTGAGGGAAGCGTGTTCTCACGCTCACTGTCGACATTGTAAACCTGAGGGAAGCATGTTCTCACACTCACTGTCGACGTTGTAAACCTGA
GGGAAGCGTGTTCTCACGCTCACCGTCGACGTTGTAAACCTGAGGGAAGTGTGTTCTCACGCTCACCGTCGACGTTGTAAACCTGAGGGAAGCG
TGTTCTCACGCTCACTGTCAACGTTGTAAACCTGAGGGAAGCGTGTTCTCACGTTCACTGTCAACGTTGTGAGGGAAGGGTGTTTTCACGCTCA
CTGTCGACGTTGCGAGGGAAGCGTGTTCTCACGCTCACCGTCGACGTTGTAAACCCGAGGGAAGCGTGTTCTCACGCTCACTGTCAACGTTGTG
AGGGAAGGGTGTTTTCACGCTCACTGTCGACGTTGCGATGGAAGCGTGTTCTCATGCTCACCGTCGACACTGTAAACCTGAGTGAAGTGTGTTC
TCACGCTCACTGTCGACGTTGCGAGGGAAGCGTGTTCTCATGCTCACTGTCGACGTTGTGAGGGAAGCGTGTTCTCACGCTCACTGTCAACGTT
GTAAACCTGAGGGAAAGGAATTCTCTCTCACATGCTGTGGGGTGCTGGCCCCACCAGGCCTCCCAGGAGACCTCCCCAAAAGTCAGAAGAAGGG
CTCCAATTGAAAGGGAGGTCCGAGATGTGCTCGCCAAAACCACCCCCAGCAAGACGCCAGGCATCCTAGCCACCCGACGCTCAGGCCGTGCGTG
TCCTCAGAGCGTGTTTTAAAGCAGGAGGGATTTGGGGTGGAAGATTGCTCCTGCAGAAATCAGAGATGAGGGGAGTAAATTAGGGAACCAGCGT
CAGGATCCAGACTTCTGTGAAAGAGCCACAGGGCTCAGCATCCGACGGGCGCACCTGGGGCTGGGCGAGTCTGGCTCCAGAAGGGGGGTGGGGC
CGGGAGCCTGAGATGGGATGAGAGGGCGGGAAGGGGCTTGTTGAGAAATCTAAGGGCCACGGGCCACCAAAAACAAACCAGGAGGTGTGAGTCA
GTGGGGAGGCTGGGTGCCAGGAAGCCTGCGCTCTGGGACATGCGTGGGGCAAGAGGGGCGCCCTGAGCTTGGCGACGCCAGCCGCGCGCCTGGG
AGGGATCCGCCTGTCCACGTGCAGCCGCCTCCGGGCGGCGTCGGCCATGCTGCTGCCCCACCGTGGCTCTGTGGCTCCAGCCGGAATGGCAAAG
CCTGCCTCCACAGCTGCCTGGGAGCGTGAGTCACGCGCCCCACAGAACACGGCGGGCAGCGCGGGCGAGGATTGGCGAAAGCCGACCGATGTGT
TTTGGCTTGTGTGGTCTGTGGGAGCGTCCTTTGTCATCAGCCTAATTGGGCAACAGATCCCACCTCACTGTGCCCTGGAGCAGCACGGGGCCAC
CACACAGCCTGAAGCCCCACTTGCGCTTCCCCTCGCTGCCGTGCTGTCCTGGCCCCGTCGCTGCCACCTGAGGCCCCCATCAAGTACCTGAGCT
CTGCGTCTGTTTTCACGCTTCTGTATGTGTAGGAAGCCCCCCACCGATGTTGCTCGGGGTCGACGTGATAAACCTGAGGGAAAGGGAATTCTCT
CACCCCGACCGACACACAGGATGGGTGTGGGGGCAAAGTGAGATCATGAACAAAAGCATCTGGGCCCGTCCTGGACACATGGCCAGGGTTAGCC
ACGGTTGTCAGCCTCCACGGAGCTGCAGTTTCAGCCTGGAGGTCTGTGGCGTGCATGGTTGGAATACTCACCAGACTCACGTGGTCGTGACTCC
GGAAGATCATGCCTTCCTGGAAACCTTGGAACGCGCTTCAGCAGAAACTCCAGTGAAGAGTCGGTGAGAGAGCACAAATTCCCAAAGGCGGATG
GAGTTCGGAAGGACCCTTCTCTGTCTCCCACATCAGGCGGTGCCTGGTTGGTGCAGATTTACCCCATGGTGGGCCGTGGATCCCTCACCTGCTC
TGGAAACTGATCTTGGCTCTGAACAAGGAGAAATGATGCCTCTCAGCCTCGAGGGATGGGTGATAAGGCAGCTTCATCCGCTGTTTTCTTTCAT
TGTTTGAGAAAAGTCATGCCCCGGAGGTGTCATTCCTGGTTGTTTATGTTGATGGCAAAACTTGGACTTTCTTGTCATACAGCAAACAGAAGAC
AATCTGATCTTTGTTTTCCAGGGGTTTAGACTTTGAACAAAACCTCTTTTGTCCAGTACTGTAGTGCATCCCTGGTAGAGTAATTCAAGCTGAA
CACAAGGTTGCATAAAAATGTGGGCATGGGGGAGCAGGGAACTGTCTTGGGATCATTCTTTGCTGTGCACAGCAGCTGCAGCCTGCAGGCAGCT
CAGCGTTAGATCTCCCCACCCCCAATGCCACCTGGGGACCAGATGTTACTGTTGGCGGAAGAAGGGGGTCCGGGGCACTGTCTCAGTCTTTGGG
GCTTCCATGGTGGAGCACTACCGGCCGTACTGTTGGTGGAAGAAGGGAGTCCGGGGCACTGTCTCAGTCTTCGGGGCTTCCATAGTGGAGCACC
ACCGGCCGTACTGTTGGCAGAAGAAGGGGGTCCGGGGCACTGTCTCAGTCTTTGGGGCTTCCATAGTGGAGCACCACCGGCTGGGCACCTTGTA
AACAACAGACATTGATTTCTCACAGCTCTGGGGGCTGGAAGTCCGAGGTTGGGACTGGTACGGTCGGGTTCTGGTGAGGACCATTCCAAGTTGC
AGACAGCCAAAATCCTGCCATATCCTCACATGGCAGGACAGGCGATTTGTCTCTCCCTCCCTGTGGGACGGCAGTCCCGTTGGATCAGGGCCCC
ACCCTGATGACATTGTTTAACCCCCAGTTACCTCCTGCAAACACCACTCTCAAGTAGAGTCACGCGGACGGGGGTTAGGGCTTCAACATGAGGT
TGAGGGGACACGAGTCCGTCCATAGCAGTGACCCCACTTGTCTAAAGCCACAGGGCTGAGCCTGCCCCCTGCCCCCCTGCCTGCCCATCCCCAG
GCCCCCGTTCTTGCCCCCAGGCCCCCCCCCGCCCACCCCCAGGTGCTCCCTGTGCTGCAGACAGAGCCCCCGAGGAGCCAGGAGCTGCCGGGCA
CCAACCACACAGGAAGCCAACAGGTGGGGCAGGCTTGTGCCTGCCACCGTGTTGCTCATGCCGATTTTTATTCTCAGATCTTGTCACCTCTGTC
TTGTAGCAGAAGGGGGCCCTGATCTGGGAGCCGTGAAGACCCCAGCTGTCCCCCGGGAGCCAGGCCACACCAAGGCAGCCAAGGGGACTACGTT
GGGAGGAGGATGTGGTCCGGGCCATTAAGAGCTGTGATGAGCCCCCAGGTCTGCCCTGGGTGAGGGGCTCTGCCTGCCCTGGGGCTGCATAGAC
CCGGCTGGGGCTTTAGAACAGGAACCGTTCGAGGGGCTGCGGTGGGTGAAGCCCACTCATGTCCTGGCTCCTGTTACCGGGTAGGGATGGCAGC
CCATTTTCCAGGCTTGGGGGTTCTTTAAGAGGCCAGGATTGTGCCGAATGCTCTTTGGCTGACGATGGCCGTGGCGGGGGCTTTGGGAGGGGAG
GTCCTGGAGGAGAGGCTTGCCCAGCTCACTGCCTGCGTCTGTGGCCAGAATTGGCACCTGCATGGGGCCTGGAAACGTCGTGCTCACCTGGGGC
GGAAGCCCCCCTCCAGGACTCAGCTCCCAACGTCACTGCTACCGAGGCAGAGACTCTGCAGCGGCATCAGAGAGAAGCTGGGCCCCAGAACAGC
ACCAGGCATGGGACGGGCCCTCCGTGCCTTCCACGGAGGATGCAAGCGTGAGGCCTGGGTCTGCGGGGACGGTTTACCTGTCAGCTCTGCACGC
CGCTGACCACTGCGCGCCGCCTCCACAGACAGTCCCCATGGACGCTGGGGCTGCCGTGGACGATGGGGCAGCCATCATGGACAACGGGACTGCC
ATGGACACCGCAGCCAGCCTGCTCTGTGGGAATATGCTTTGTAAAGCCAGCCGGCCCTGAGGCCAGCACTCGCCTTCCACACTCTGCTCTTTGT
AACGAGTTTCATAAACTCACCCTCAGGTGATTGGTGTTTCTGTTGCACCGTTGCTTAGCCTGAAAATGAGCTGCCCTTTTTCTGAGCCCATCCT
TTCGCCACCTCTACCCCAGCCCCTCATGGCTGATATCAGGAGGTAGCCAGAGGCCGCCTGGTGCAGGGTGAGCCCGGAACACGGGCAACAGCTT
GAACTGGGCCTGTGCGGCCCCCTCCACGGGCGACAGCGTGAACTGGGCCTGTGCGGCCCCCCTCCACGGGTGAGACTCCCAGGCGCTCTTGCAG
ACGCTCACTCACTTTCTCACTGATAGGATGCGTCTTACGAGGCTCTGATTTACAGAGCAGGCATCCATCTTCTCTGAAACCCACACAAAAAGCA
CAGTTTAGGCCAAGCCCAGGTGCAGTATGTTCGTGTGTTTAAGCTTCTTAGATGCTGGGGTGTGGAAGCAGCCGTGTGGCTTTCCTTTGCTTGT
GAAGCTGAGTTTCCACTTTCCAGCTTCTCTCTCTATTGGGAAGGCTGCAGCTCTCCAGGACATGACTTAGCAGAGAACATCCCGCTGCATCCTC
ATAGGCCAGCGGGTCAGGCGGGGCCATTCTGAAACGTCCCCTGTGGGTGCCGGGTGCCTCGCGCATCCCTGCCGTCCACCGCCCTTTGTCCCTG
GCTGTGCTTCGCGGCAGTGCCCCTTAGGGCAAGGACAGGGAGATGGGGCAGTCGCCTGGGGAAGGCCCGGTCCCAGTGGGAGGCAGCCTCAGAG
CAGCTGGGATGGCACAAGACGCCGCTGCCAGGGCTGAGAAACCATCCGGCCAACCCCAGACTTGAGCCAGACTTCATGGGCAAGGCTGGGAGAA
GCCGCGTGGAGACCCCAGCCCAGGGAGGGGCACTGTGGCAGGAACAGGGCCCGCACCTGCAGGGTCAGCCCAGGCCACCTTCCAAGCACGGTGA
CCACGTCGCAGCTCATGCTGCCAGCTCCGGGCCAAGGGACCTGGACTGGCTCTCCAGACTCACAAGATGCCGTCGTGGGTGGGGCACGTGCGGT
GATCCCAGGTTTCAAGTAGCTGCCCCCAGCTCCCTGCAAATAGCTCTGGGCCGAAGAGAAGCAGTGGGCAGGCACACGAGTTTATTCCTGAGAG
GAGCGTGTTGCCACAGCCTCTGTGGACTGGAGCCCCAGGCTTCAGAAAGAGAGGGAGCGCCTGCCTGTGGGTGGGGGGACAGGAGCTGCCAACC
TGGCGGGGTCCCAGCGAAGCGTCCACTGGGCCTTCTGCTTGCCCCTGGGGGTGGTGGATGCATCAGTGCTGGGCCTGCTCCGAACCCGCAGGGC
AAGCAGCCCGTGCGTCCCGATGCCACTGCCGGCCGCCAGCATCCTGGGGAGGTGGGTCGCGTGTCCCAGAGCCCTGTGCCCCTCGCCACCACCA
TCCTAGGTCAGGACTTCCCCAGGAAGAATCCAAGTGGCTGGAGAGATGCGGGGGTTAATGCCGGTACCACCGCGGTTTTAAACGGGGCCGTGTC
TGAGCCGTGTTCTTGACGTTCTCCAGCCAGCAGCAGCAGACGCCACCCGGTTGCCCCAGCCCCAGCGGCTCTCGGGCATAGCCGGCCTTGGCCC
GTGGGGCGGCTGATTTACAGGACAGCTGTGCACGGGGCCTCTAGTGTCCGCCAGAGCTGGTGGGTGAGAGTTTTTAAAGCGCTCGGGACACTTC
ACAGAAGAGGCGCACTGGCCCCCATGCCTGGCCATGTTTCTGGGCCCTGGCTGAAAATCCCGAGCAGCGGAGAGAGGATCCCAACAGGGTATTA
ATTTAACCTTCCAGCCATGTGCTTTTGCTTAAGATCTAAACTCTGAAAATTACCAGCCTAATTTAGAATGGAGGCTTGGCCATTCGGGACACCA
GCATCTTCTGAGCGGGAGAGATGTGGGTGCCCACCGGCGTCTCACTCCCACATCTGGGCCTGTTTCCAGTGACCCCTGCACCTCTCAAAGGCGC
AGGCCTGAGTTCACAGAGAGCACTTGGCCAAGCCCTGCCTCTGGGGCTCCCCATCGTCTGGCTTTGTCGTGGACTCATCATTTCTGGATTTACC
ACATGCTTCAAGCTGGAACAGCAGGAAGGGGCAGTTCCTGAGGCCCAGCTGCGTCCGCAGAGCTGGGGGCGCCATGGCCTAAGGCAAGACGCGT
GCCCTGGATGAAAATGACTCCAGCTCTGTAGTCTCCAGACAGCGGTTAGAGCCCCACCCTCCACGGACACCGCCTCCTGCCACCCCTTCAAAAA
AGTGTTGCAGCCCAGGGCCCAGATATTCAATTTTCTAATGTAAAACGAGGCGGGTAGGGTTTCCCAATCAACATGAGGGCGCCGTGTCCTCTGC
AGCCCCCTGACTTGCACAGGCAGTCCTGGGGCTCTGCCCCATCTGTGAACCCTCAGCTGAAGAGGGCAGCCCCAGAGGCAGCCACATTCAGGTG
GCTGAAGGCAGAGTCCTCCTCCCAGGAACCGCCTCGGCGCTGCCAAGGCCTGTGCACCTTCTTCAGCCCGAACTAAGGAGAATGTGCGGGACTG
AGAGCCATAGACCCTCGTGAGCCAGGCTTGAGCGTTGTCTTCAGATGGGGCCCAGTGGGCCTCACTGTTGTGACGACTGAATGTGACGTCTGGG
GTGTAATTCCAGCGTGACCTGGTCCTGGGGTGTCACTCCAGCGTGACCTGGTTCCTGAGGTGTAATTCCAGCGTGACCTGGTTCCTGGGGTATA
ATTCCAGCGCGACCTTGTTCCTGGGGTGTCATTCCAGCGTGACCTGGTCCTGGGGTGTCATTCCAGTGTGACGTGGTCCTGGGGTGTCACTCCA
GCATGACCTGGTTCCTGGGGTGTCATTCCAGCGTGACCTGGTCCTGGGGTGTCATTCCAGTGTGACCTGGTTCCTGGGGTGTCATTCCAGCGTG
AGCTGTTTCCTGGGGTGTCATTCCAGCATGACCTGGTTCCTCAGGTGTAATTCCAGCGTGACCTGGTCCTGGGGTGTCACTCCAGCATGACCTC
GTTCCTGGGGTGTAATTCCAGCGTGACCTGGTCCTGGGGTGTAATTCCAGCATGACCTGGTTCCTGGGGTGTAATTCCAGCGTGACCTGCTCCT
GAGGTGTCACTCCAGCGTGACCTCGTTCCTGGGGTGTAATTCCAGTGTGACCTGGTTCCTGGGGTGTAATTCCAGCGTGACCTGGTTCCTGGGG
TGTCATTCCAGCGTGACCTGGTCCTGGGGTGTCATTCCAGTGTGACGTGGTCCTGGGGTGTCACTCCAGCATGACCTGGTTCCTGGGGTGTCAT
TCCAGCGTGACCTGGTCCTGGGGTGTCATTCCAGCGTGACCTGGTTCCTGGGGTGTCATTCCAGCATGAGCTGTTTCCTGGGGTGTCATTCCAG
CGTGACCTGGTCCTGGGGTGTCACTCCAGCGTGACCTGGTTCCTGAGGTGTAATTCCAG
HUMAN SEQUENCS—mRNA
GAATTCCGGGCCGGGAGAACCGAACCCCTGGCGGCCGCGACCCCGGCTCCCGCCCCGGCCCCGGCCCGACCCGCCATGACGGGGCTGGAGGACC
AGGAGTTCGACTTCGAGTTCCTCTTCGAGTTTAACCAGCGCGACGAGGGCGCCGCCGCGGCCGCCCCAGAACACTATGGCTATGCATCCTCCAA
CGTCAGCCCCGCCCTGCCGCTCCCCACGGCGCACTCCACCCTGCCGGCCCCGTGCCACAACCTTCAGACCTCCACACCGGGCATCATCCCGCCG
GCGGATCACCCCTCGGGGTACGGAGCAGCTTTGGACGGTGGGCCTGCGGGCTACTTCCTCTCCTCCGGCCACACCAGGCCTGATGGGGCCCCTG
CCCTGGAGAGTCCTCGCATCGAGATAACCTCGTGCTTGGGCCTGTACCACAACAATAACCAGTTTTTCCACGATGTGGAGGTGGAAGACGTCCT
CCCTAGCTCCAAACGGTCCCCCTCCACGGCCACGCTGAGTCTGCCCAGCCTGGAGGCCTACAGAGACCCCTCGTGCCTGAGCCCGGCCAGCAGC
CTGTCCTCCCGGAGCTGCAACTCAGAGGCCTCCTCCTACGAGTCCAACTACTCGTACCCGTACGCGTCCCCCCAGACGTCGCCATGGCAGTCTC
CCTGCGTGTCTCCCAAGACCACGGACCCCGAGGAGGGCTTTCCCCGCGGGCTGGGGGCCTGCACACTGCTGAGTTCCCCGCGGCACTCCCCCTC
CACCTCGCCCCGCGCCAGCGTCACTGAGGAGAGCTGGCTGGGTGCCCGCTCCTCCAGACCCGCGTCCCCTTGCAACAAGAGGAAGTACAGCCTC
AACGGCCGGCAGCCGCCCTACTCACCCCACCACTCGCCCACGCCATCCCCGCACGGCTCCCCGCGGGTCAGCGTGACCGACGACTCGTGGTTGG
GCAACACCACCCAGTACACCAGCTCGGCCATCGTGGCCGCCATCAACGCGCTGACCACCGACAGCAGCCTGGACCTGGGAGATGGCCTCCCTGT
CAAGTCCCGCAAGACCACCCTGGAGCAGCCGCCCTCAGTGGCGCTCAAGGTGGAGCCCGTCGGGGAGGACCTGGGCAGCCCCCCGCCCCCGGCC
GACTTCGCGCCCGAAGACTACTCCTCTTTCCAGCACATCAGGAAGGGCGGCTTCTGCGACCAGTACCTGGCCGTGCCGCAGCACCCCTACCAGT
GGGCGAAGCCCAAGCCCCTGTCCCCTACGTCCTACATGAGCCCGACCCTGCCCGCCCTGGACTGGCAGCTGCCGTCCCACTCAGGCCCGTATGA
GCTTCGGATTGAGGTGCAGCCCAAGTCCCACCACCGAGCCCACTACGAGACGGAGGGCAGCCGGGGGGCCGTGAAGGCGTCGGCCGGAGGACAC
CCCATCGTGCAGCTGCATGGCTACTTGGAGAATGAGCCGCTGATGCTGCAGCTTTTCATTGGGACGGCGGACGACCGCCTGCTGCGCCCGCACG
CCTTCTACCAGGTGCACCGCATCACAGGGAAGACCGTGTCCACCACCAGCCACGAGGCCATCCTCTCCAACACCAAAGTCCTGGAGATCCCACT
CCTGCCGGAGAACAGCATGCGAGCCGTCATTGACTGTGCCGGAATCCTGAAACTCAGAAACTCCGACATTGAACTTCGGAAAGGAGAGACGGAC
ATCGGGAGGAAGAACACACGGGTACGGCTGGTGTTCCGCGTTCACGTCCCGCAACCCAGCGGCCGCACGCTGTCCCTGCAGGTGGCCTCCAACC
CCATCGAATGCTCCCAGCGCTCAGCTCAGGAGCTGCCTCTGGTGGAGAAGCAGAGCACGGACAGCTATCCGGTCGTGGGCGGGAAGAAGATGGT
CCTGTCTGGCCACAACTTCCTGCAGGACTCCAAGGTCATTTTCGTGGAGAAAGCCCCAGATGGCCACCATGTCTGGGAGATGGAAGCGAAAACT
GACCGGGACCTGTGCAAGCCGAATTCTCTGGTGGTTGAGATCCCGCCGTTTCGGAATCAGAGGATAACCAGCCCCGTTCACGTCAGTTTCTACG
TCTGCAACGGGAAGAGAAAGCGAAGCCAGTACCAGCGTTTCACCTACCTTCCCGCCAACGTTCCAATTATAAAAACAGAACCCACTGATGATTA
TGAGCCTGCTCCAACCTGTGGACCGGTGAGCCAGGGGTTAAGTCCTCTCCCAAGACCATACTACAGCCAGCAGCTCGCGATGCCACCCGACCCC
AGCTCCTGCCTCGTGGCCGGCTTCCCGCCCTGTCCGCAGAGAAGCACCCTGATGCCAGCGGCCCCTGGCGTGAGCCCCAAGCTCCACGACCTTT
CTCCCGCTGCCTACACCAAGGGCGTTGCCAGCCCGGGCCACTGTCACCTCGGACTCCCGCAGCCGGCCGGAGAGGCCCCCGCCGTCCAGGACGT
GCCCAGGCCAGTGGCCACGCACCCCGGCTCGCCCGGGCAGCCACCCCCGGCCCTGCTGCCACAGCAGTAAATGAAATAATACGAAATGACCTCT
CCAGCACGAGCACCCACTCCTAGTTGCCACATTGGAGCACTCAGTTCAGCAGGGGTATGCTGACTTCAGCAGACAAAGACTTTTGAATAAATAA
ACTGAACTCACACCTGGTACCACTCAGAACCTCCAACTGACTGAATGCCAGGAGCTGAACATTAATATGTGCAAAGATTGGCTCTCCAACAAGA
AGGAAAGCAGGGAGGAAGGGAGACCACTGTGTCGCCTGGAGGAGAAGTCATCTCATGACAACAGAAGGGAGGTGGCCGGGCTGAGCACGGAGAC
CCACCGTGCAGGGGCCTTTCATGGGAACGGCCCACACGCAGTTTGACCCCACGCCCAGCCCTTCTGGCACCCCTGGGGTTCAATACTGGAAGTG
CCTTATTTAACCAGACCATCA
HUMAN SEQUENCE—CODING
ATGACGGGGCTGGAGGACCAGGAGTTCGACTTCGAGTTCCTCTTCGAGTTTAACCAGCGCGACGAGGGCGCCGCCGCGGCCGCCCCAGAACACT
ATGGCTATGCATCCTCCAACGTCAGCCCCGCCCTGCCGCTCCCCACGGCGCACTCCACCCTGCCGGCCCCGTGCCACAACCTTCAGACCTCCAC
ACCGGGCATCATCCCGCCGGCGGATCACCCCTCGGGGTACGGAGCAGCTTTGGACGGTGGGCCTGCGGGCTACTTCCTCTCCTCCGGCCACACC
AGGCCTGATGGGGCCCCTGCCCTGGAGAGTCCTCGCATCGAGATAACCTCGTGCTTGGGCCTGTACCACAACAATAACCAGTTTTTCCACGATG
TGGAGGTGGAAGACGTCCTCCCTAGCTCCAAACGGTCCCCCTCCACGGCCACGCTGAGTCTGCCCAGCCTGGAGGCCTACAGAGACCCCTCGTG
CCTGAGCCCGGCCAGCAGCCTGTCCTCCCGGAGCTGCAACTCAGAGGCCTCCTCCTACGAGTCCAACTACTCGTACCCGTACGCGTCCCCCCAG
ACGTCGCCATGGCAGTCTCCCTGCGTGTCTCCCAAGACCACGGACCCCGAGGAGGGCTTTCCCCGCGGGCTGGGGGCCTGCACACTGCTGAGTT
CCCCGCGGCACTCCCCCTCCACCTCGCCCCGCGCCAGCGTCACTGAGGAGAGCTGGCTGGGTGCCCGCTCCTCCAGACCCGCGTCCCCTTGCAA
CAAGAGGAAGTACAGCCTCAACGGCCGGCAGCCGCCCTACTCACCCCACCACTCGCCCACGCCATCCCCGCACGGCTCCCCGCGGGTCAGCGTG
ACCGACGACTCGTGGTTGGGCAACACCACCCAGTACACCAGCTCGGCCATCGTGGCCGCCATCAACGCGCTGACCACCGACAGCAGCCTGGACC
TGGGAGATGGCGTCCCTGTCAAGTCCCGCAAGACCACCCTGGAGCAGCCGCCCTCAGTGGCGCTCAAGGTGGAGCCCGTCGGGGAGGACCTGGG
CAGCCCCCCGCCCCCGGCCGACTTCGCGCCCGAAGACTACTCCTCTTTCCAGCACATCAGGAAGGGCGGCTTCTGCGACCAGTACCTGGCGGTG
CCGCAGCACCCCTACCAGTGGGCGAAGCCCAAGCCCCTGTCCCCTACGTCCTACATGAGCCCGACCCTGCCCGCCCTGGACTGGCAGCTGCCGT
CCCACTCAGGCCCGTATGAGCTTCGGATTGAGGTGCAGCCCAAGTCCCACCACCGAGCCCACTACGAGACGGAGGGCAGCCGGGGGGCCGTGAA
GGCGTCGGCCGGAGGACACCCCATCGTGCAGCTGCATGGCTACTTGGAGAATGAGCCGCTGATGCTGCAGCTTTTCATTGGGACGGCGGACGAC
CGCCTGCTGCGCCCGCACGCCTTCTACCAGGTGCACCGCATCACAGGGAAGACCGTGTCCACCACCAGCCACGAGGCCATCCTCTCCAACACCA
AAGTCCTGGAGATCCCACTCCTGCCGGAGAACAGCATGCGAGCCGTCATTGACTGTGCCGGAATCCTGAAACTCAGAAACTCCGACATTGAACT
TCGGAAAGGAGAGACGGACATCGGGAGGAAGAACACACGGGTACGGCTGGTGTTCCGCGTTCACGTCCCGCAACCCAGCGGCCGCACGCTGTCC
CTGCAGGTGGCCTCCAACCCCATCGAATGCTCCCAGCGCTCAGCTCAGGAGCTGCCTCTGGTGGAGAAGCAGAGCACGGACAGCTATCCGGTCG
TGGGCGGGAAGAAGATGGTCCTGTCTGGCCACAACTTCCTGCAGGACTCCAAGGTCATTTTCGTGGAGAAAGCCCCAGATGGCCACCATGTCTG
GGAGATGGAAGCGAAAACTGACCGGGACCTGTGCAAGCCGAATTCTCTGGTGGTTGAGATCCCGCCGTTTCGGAATCAGAGGATAACCAGCCCC
GTTCACGTCAGTTTCTACGTCTGCAACGGGAAGAGAAAGCGAAGCCAGTACCAGCGTTTCACCTACCTTCCCGCCAACGTTCCAATTATAAAAA
CAGAACCCACTGATGATTATGAGCCTGCTCCAACCTGTGGACCGGTGAGCCAGGGGTTAAGTCCTCTCCCAAGACCATACTACAGCCAGCAGCT
CGCGATGCCACCCGACCCCAGCTCCTGCCTCGTGGCCGGCTTCCCGCCCTGTCCGCAGAGAAGCACCCTGATGCCAGCGGCCCCTGGCGTGAGC
CCCAAGCTCCACGACCTTTCTCCCGCTGCCTACACCAAGGGCGTTGCCAGCCCGGGCCACTGTCACCTCGGACTCCCGCAGCCGGCCGGAGAGG
CCCCCGCCGTCCAGGACGTGCCCAGGCCAGTGGCCACGCACCCCGGCTCGCCCGGGCAGCCACCCCCGGCCCTGCTGCCACAGCAGTAA

TABLE 39
MOUSE NOMENCLATURE
ICSGNM Syk
Celera mCG3409
HUMAN NOMENCLATURE
HGNC SYK
Celera hCG37721
MOUSE SEQUENCE—GENOMIC
AACTTTTAATAAAATATAGTTGCATACAGGTGAATATAGTCTTAGCATACAAATAACTGGTTTTCTCTAAACTGTAGCATTCCTGGATGCTTTG
TGAAAGAGATTGGGTCTTTTCATGGAAGTGTGGGGAGAGGGAAGAAATTACTTAGAGCCACTGAACTCTTGATCATGGATTCCTTATTTAAGGA
TTCACTGTTTCTTGATCTTGGGGTGAATAACCCAGTCAATGTGTTGCCTTGCAAGCATGAGGACCTGGGGTCAACCCCATGCAGGCCTACAGAA
CAACACCAGTAGCAGCAGGCATGGTGGTTCATACTTCTCAGTCCTGGAGAGGTTTGAACACAGGGATTCCTGGGGCTTCCTGGCCAGCCTAGCC
TGTTCGGAAAGCGCCATGCCAGTAAGATAGCCTGTCTCAGGAAGCAAGACAGATGGAGGGGGTCTGAAAAACAAAAGCTAAGGTTGATCTCTGT
TGGATGTGCATGCACACACACATACACACACACACACAAACATATAATCCACACTCGCATGCACACACACACACACACACACACACACACACTT
CACTAAAATGTATTTGCAAGTCTAGTCAGTTATAGCACTCTGTCATTTTTTGTGGACATGCACGGAATGATTTCATTCTGTTTCTGTGAGAAAA
CCTGACCAAAAAGCAACTTCTGGCAAGAAAAAGGTTTATTTGGACCCTTTCAGGTCCCAGTCTGTCATTGAGAGGAGTCAGGGTGGGAACTTAA
GCAGGAGCTTGAAATGAAACCATGTAAGGATCCTGCTTGCTGCTCACGTTCTGACCTGTGCTTCTGTAGTTCTCTTATACAGCCTAAGATTGCT
TGCCTAGGGATTGTGCCGCCCATGGTGGGCAGGACCTTCCCACATAAACCATCAATCAAGACAATCTCTCACAGACATGGATGCAGGCCAGTCT
GACCCAGACAATCACTCAATCGAGGTTTTGGATGACTCCTTGCTTGTCAACTTAACAGATGAGGTTAGCTAACCAGGACAGTGGCCCTCTTCTT
GTCTCACAACATAAACAGTGCCCATTTTCTTGATCTATTTAGGGCCACGTTTTAATAACAGATTTTTTTCCGTGCTTTTTGTTGGTGCCATCCC
TACTGAAAATAGTCCTAAGCGTGGGGCTAAAGTGCTGCCTACTGTCCCTAAACACAAAAAGGCTGCTATGTGCTTCATTAAGTAAATTCCCATG
TTGGGTGAGATTCTTACAAGTATTGGAGTCCTGCCCAGAACTCAATGTCAGTGTATTGACAATGGATGGGAGTTGTTTCTTTAAACAGATGCAT
ATCTGCTACAAGGTTGTTCCTTGTCTGGCTGGCAGAAGTATAATCAGGGGATCTCCAGGATTAACTTTAAAAGTTCCCTGAGAAAGAGTGGTTG
AAATTTTGTAAATTCTAGCACTTCTGTGGGAAGTATCTACGGCTCATCATAGGAAGTAACTATATTTAGTGTCATGCATTGGAAAGTAGGACCA
AGGTCAGGGGAATGGATGAAATGGAGAGAGGATGTGTGAGCCCTGATGCTTGTGGTGGTAAAATAACCTGAACACTGGGGTTCATCCCTGACCC
TAACCACAGTAGATGGTAAGTCCTAGGTTCAGATGGTGCTGCTCTGAGGGGATCTAGGCAGAGTACTCTGGCCCCTGGACTTTCTGTAAACATG
GAGAAGTGAGGGGAGATAGGGGTAGGCTGGCAGCATCCTGGAACACACCCTCAGCCATGTGCAACTTCTTTCCTCACCTCAGTTCTCCTTCTCT
CTTGGAATCTGGGTACCAGAAAGTCTTCTCCAGGCTACCAGAGCCCAGACCACTGGTTTGTTGACAGTATGTCGAGATAGTTGTTAGTCGCTGG
TTGTGCCTCTGGTTTGCCTTTGGGTATCTGCTTTGGGTACCAGGATCTAAGGAGCTGCCTGTGTCTGCCCTGAGAAATTCAATTCTTTAATTAG
CTCAATAGCCGGGCAGACACGGCTGTTGCTCAGTGTCTCTGGAGGTCATTGACTGACAAGATAGTACTACATTGGTGCCTGTGATAAGACAAAG
GTCACACAGTCCTCCTTAGTCCTGGGACTGGGGAACAGGCATGTGATTGTCTAATGGGAAAGGAAGGTCAGAAGTCAGTTGGTGAGTAATGTGC
TTGCTCTGCAGCATGGAGACATGGGCTGACAATCACCCATGACAAGGGCAGGGCTGGAAAACGTAATCCCTGGTCTAGGGAAGTAAAGCTAGGC
AGAGCCCTGGAGCTCCGTAGCCAGTCTACCCAAGCCAGGGAGCTCTAGGTTTAATGATAGATTCTGTTACAGAAAACAATGTTAAAAAACGAGA
GGAAGATACCAGATGTTATGTGTGCATAGATTATGTGTTCTCACTCTCTCTCTCTCTGTCTTCTGGCTGCCTCTCTCTCCTCTCTCTGTGTCTC
TCTGTCTCTGTCTGTGTCTCTCTCACACACATACATGCACACACACATGCAGACATGCTCACATACATACACATGCACACTCGCACGCATACAC
ACGCAATCACATGCACACATTAGTGCGAAATGATTCCCCGTAACTGGTTATGACTGCTGACCAATGCTCAGGGCTAGCAGAGGGAGAGGAAAAC
CACCCAGCATGTAGGCAGCTTCCTGAAAGCGTTCGGCTTCCTCCATTAGCTAAACTACAGCTTTGGAGGAACGGCACCTTTAGGTAGGAAGAGT
GCCTTAGCCCTTTGATGTCTGCAGTCTCTAAGCGCAAGCCCCTCTCATGCTCCACTCTGCACACCCATGGAGTTTGCTTGACAAATTCAGGCAG
CCATTGACCTCTCCCTATAGCAGAGGGAGGTGAGAATCAACTCTGAGGGCTTCAGTCTGAGCTAGAGGCACACTGACAGCTGCCATGCCTCTGG
ATTCAGCATTTCTTTCCTGGAGTCCTAGGGCTCCCAGGAAGCCAGTCATAGACTCGTGGGCATCTCTGAGGTGGGTGTCTGTTAGTGTCTGTAA
GACTGCAGGAAGTGTGCTCTGGTTAGCCCCTGGCATAAACCTAGCAAGGATCTAGCCTGTGAAGAAGCATGGCCACGCTCTGGACTTGACCGGT
ACAGTGTTCTGAGGGTGGGATGACACCATTTGGTACCCAACTGTGTTCTGAGGGTGGGATGATATCATTTCTGAGCATCCTGATGTGTCTGTCC
CTGTTTGGTGAGCACCTCCCTTCCTGAAAATCATGTGTTTCTATTCCTCCTCTTTATATCTGTGAAACCCACTCTTCCTATCTGCGCAGAGCTT
GCCATCCCAAGGTGCCAGTTGCCATGGTTCCCATCTTCCATGCATAATCAAAGCACTACAGTCGCTCTCACACTGCCCTCCATAGTCATAGGAG
AGCTGGGGAGGTGCACAGGTGTCAAGGTACTGCAGAGCCAGACAGGACACCTGCAGAGCTTGCGTTTAGCTTCTGCTCTGCATCCCTGCTGCCT
TCTAGGCTTAGAGAGCATCATAGCCAAGAGTCCCTCTAGGCCTCCTGACCTCTGCTGTTGTACAGCACCATGCTTAGAGAAACAGAGCTGGCAT
CCTCCTCAGATTGGCTGCTCCTGCTTGCAGGAGTCCATCAGGAGCCAGTCAGTGGACTGCAACCTCAGAGAAGGAAGGTGTGGTTGGGGTCACT
GGACCCTCAAGCAATACTATAGACACCCAGCCTAGTCATCTGTAATTGTGCCCCTACCTATGTGTACATATGGCTTGGCATCTCAGGGAAGGGG
TGGAGTGTGTAGGATGGGAAAGACTAGCTAGAGAATGGGAGTTCTAGCTATGTAGCCATGGGGAAGCCATCATCCATGCTGGGTACTGATCTTC
TAGTGTAGTTGTTTTAGGGTCCCCTACCCATAAACCCCTTGGCCATGTTCTATAACTAAGCCTAAAAAACCCGTTGGTCGCTTAGAGTGAATTT
TTGTGGACTCAAAATTCTTTGGACCCTTATGAGGAAGGGGGAGATGTTTATGTCATCTCTGGGGAGGAGTTTCCACAACGTCTGCTCATTGTTG
AAAATGGTCTTCAAATAAATGAGGGTACATTGAGTTATTTTAATAGATTAGTGAAACCAAAGGTTCTAAGACAGGCTGCATCAAATTTGTGCTC
ACGTCACTGCAACTAATCTAGTGGTAGTAGAAGAGAAGTCCCCCCCCACCCTAAACACACATACCAAGGACTGGTCCAGTCTCTCTGTCAAGAC
TACCCAGGCTGAGCAGCTCCGAGATGCTACTGTGAACAGTTCTAGAGAACCTAATGGGTGGGATGGGGTGGCAATGCAGATGTCCTCGTATGTA
AATAGAGGGTGAGGAGGGTGTCAAGACCACATGTGGAAGAGAAACTTCTCATCATTCTCCGGGAGCTTAGGGCAGATGCTGGACAGCTGGGACC
ATCCCCAGCTGTGTTTGTTGATCCCTGGCCTACACCAAGGAGATCAGAGCAGCAGCTTCAGGGTTGGTGATATATATATATATATTTATTTATT
TAAAGAAGCTTATACAACATGAAATTATGTGGGGAAATTAAGAAAAATATGCACATGCTGGAGATACTGACGAATAGGAAGAATGTATTGACTG
CCTGTAGGCAGGCCTTTGAGGTACTTAAGAACAATATATGCCAATGATGTCCTAGGGCTTCAGAGAAAAGGCAGGGAATCCAATGAGACAGCCT
TTCATTACTGAAGCATGGAGCAGGTGACAGAGGTCCCTTCCTTGAAAAAACCATGAGTTTGTGTAAGACATGACTGAGCTCCATCAACACAGAC
ACTGGGCTAGACATCTGGACAGCTGGTGCGCTGCTCTCCAGGATCCTCTGGACTTATACAAATTGGGCTTTTAGCATTTTCATTAGGAAAATAG
CCTATAGCCACAGTAAACTCTCTAAAGAAGAGCATGTGTCCGTTAGGCACATAGTTTGTGGACATCTGGTAGGCCCTGAAACATGAGCCACCGA
GTTAAAGTGTCCAGACCTTTGAAACTTACATTTTCATATAAGGCAAAGATGGAAGCAATCTATAAGGGAAATGGATACTTAAGTGAATTTAAAT
GAGGATGTGTTGGGGATCAATTTGAGGGCTAAAGGCACACACTGAAAAATCAGATCATGAGGCTATCAAGGGGGATGGGAGGATCTTAGACCAC
AGGCCTGGGAAGGAGGAAAGTCATTAGCGAAAATGCTGTAACCAAGAGAGGATAAGCCTGACATCCGAGGCCTGGAAGGACAATGTGGGACACA
GCTGGAGTGGCTGGAGTCAGGCCAAGGAGGGCCTTGTAGGCCGGGGCTGTACTTGCTAAAGAGAAAATGGACAAAGAATGAGGCCCAGAAGAGA
GGACCAGGTAAAACTGGGAGGTGGGCATTACACAGGCCGTGTTGGGAATGGGTGAAGAGCACCAGTGGCTTGGGGAGCCATTTACAGACTCTGT
GTTGCTAGGAAACTCCCTCAAGACCCTTCAGCCATTGGCCACATTGGTGAAGATTGGGACTAGTCTACTCCATGTGTGTCCATGCTTAGAGAGA
GAACAATCTTTTATGACTCTAGGGATGATCTATGGAAGACAGGAGAGAGGGAAGGCTCAAAACACTCTTCAGTATCATCTTCACTGGCTATTGC
AGTTGCTATCACCTCTTTTAGGGAAGAAAGCAATGTAGTGGTCCCCCCAGATGAGGGTTCAGGGTAAGGAGTGGAGTTGGGAAGGTCGAGCTAC
CTCTTGGGCATCCAAGAGTAATTTAGATGAACCAACACATAGGGTTGACCTCACTAGAAAGACTGGATGTCCATTTAATCAGCAAATTACGGTC
AGAAAAAAAAATGAATGACCCTCCACCAAACATCAAGAGCTGCTCCAGGCATGGAGGAATTATGGTTCCATTCATATCTTCCACAGCCAGCTCT
ACTGATATGTTAAAGGGAGGTCACCTTGTTACACTGGACCATGGAGAAGACTGAAGCAGGGAGGGGAGACAAACACCAAGGGGATTTCAAAATA
TGAAAGAAAAAAAATCCCCTCAGTCTGGGTGGTAGCTTAGTGGGTAGGGTGCTTGCTTTGCGGGGACAAGGGCTTGAGTTCACTAACCAGAACC
CATGCAAAAGAGCTAAGCTTGGTGGCTCACAAGTGTTTGTAATCCTGCAGGGAGTCTTCAGCACTAGTTCTCACTGGGAAGGCAGCAAGGAGGC
CAGAGTTTATTTATCTTTGGTAATTCCTCTGCGCATAACTCTTTGCTTTTCCAAGGCCAAAAGCCATTGGCTTCCAGCTACAAACTCTTGCTAG
CAGCAGCAGAGAACTGAGAAGAAAAATGGCTTAATAGCAAGTCCTTCTCTCTCACCAGCTCAGGAGCTCCTGGCTGGTCAGGGGAGAGGCTGGG
AGCTTATCAACTCTTTGTGAACCAGAAAGAAAAGAAAAGGAAGAAAGGGAGACATCAGTCACACACACATGCAGACTTTGGATGAAAAAAGAAA
AGAGGCTTGATTCACTTCACACACACACACACACACACACACACACACACACACACACACACACCTTCTGATTGGGCCCGACTAACTGTCTCAA
CTCCACAACTGTTCATCCATACTTACATACAGACATCTATACTTATATATTCATGCACACACACACATACACACATTTAGTAGAGCAAAAGCTC
CCATGAGCAAGTTCCATTGAGAAAGTGCCAACACAGAAAAAAAATCACGCATTCTGGATGAAAAATAAACTCCAATCTTTATTGCTCAGAAAAA
GAAGAGGCTTCTGCCTTTTTATTGCTGAAAGTAAGGAAAGGCTCATAAGTAAGAAAATCTCATCCTCTTTATTGCTGAGAAAAATGCCTCACCC
ACCCCCCTCTCTTGCTGAATGAAAGAAGGCTTGTGTCCTACTGACATCGTTCTTAGTTCTTACACTTTCTCAGGAGAATAGATGGCATGGTTTT
CCAAGCACCTTTACATTCTGAAAAGGGTTCATAGCGAGTTCAAGACACTAATTCACGTCAATAAATCGAACAAGAAGTTACAGCAAACGTGTTT
CCATGTGTTTCCATTAAGACTAGTATCTAAATAAACATTCACTATCTAGCTTCTAGATCCATAAGCTCATTAAAAGTTTAAAGCAATAATTCTT
ATGGTGTAAGTTAGCATGGTTTTGTCAAGAGGCAAATTATTTGCCTTGTGTCTCATTAGTTTTGAAGTTTGTATAGGTAAATCCAGTCAATATT
TTATCCTCTCTCCTTTTACCTGCAGTAAATTTCCCTTCCCAGTTTATGACCTTCAGTTATCAGATAATGGTTTCTCAGCTTGCCTAGAGGGAAT
ATTTTTTTCCTCATCATAAATTTGTTGTAACCCTGCTTCCTTATCTTAGTGCAATTAGCCTGTGACACATGAACAGTTACAGAACTCTAATTGC
AGCTTCCATACAATAGAGGATTCAAAATTACTTGTATAAATTTAGGAATTCTATATAACCATTATAATCATTATTAGGAATTACAAAATCATCG
GTTTGTCTACATAGCATTACTACAAGAGAGTACATCCTGACGGATCTGCAGAAAATCTGCCCAACAGACTCCAGGGTGCCCAGGAGTCATTAGA
ATACGCAATAATGACAGGAAATCTCTTGTAGTCTTGCCTTTCTGAGCTCACCCATCACAGGCCATGCAAAAAATGGTGGCCCACTCCAGGAAAG
AGACAGGAGGATCTTGGGATTTGCCAACCCGCCAGACTAGAGTATCTGGCAAGCTCCAGGTCCAATAAGAGGACTGTTTCATAAAAAAGGAGAG
TGATTGAAAACAGCCTCTGAAGTTGACCTCTGGCCTCTAAATGTAAACATGCAGAAGCATCCACATGAACTAGCATGTGTGCACACATACATTT
CCAIACTCCTAAGGTCTTGCATGCATAATTTACATCCTACAAGGCCTCAAAAGAGTGAGAGATTTACTGTGTTCTGATGGCTCCCCATATACCT
CACGGCAGCAGAGCTACCTGTAAGGTGTGTGCAGAGGTAAGTACTGGCCAACTCTGGGCACAGGTACAGTGAGGTGGAGTAGGGGGTTGCTTTG
TCAGGACTGGGGTCTTACTACTTTCCTCAAGTTAATGCTTTTACAAAGTGGCTTCACACACTTGTCAATGTCAAACTGCGCAAAGATAGTGTTG
CACTGAGAAGGAAGAGCAAGTGTCTCAGGACTGTTGAGGGTGATAACAGCATTGTGAGCTATGAACTCTCTCTGCCGTCAGAAGAGGGGAGCTC
AGATGTGACATGCAAGCCACAGTCGTTGGTACAATGAGTTCAGTGGTGCCTGGAGCAGCACACGTCTCAGGACACCATGTTACCTGTGCCCACA
TGACATCTTTGAGTTGTGCAGCCACATGGGGGAGGATGACTTTCAGAGAGAAGGCTGTTCATCTAACTCCATGTGGCATGAGTTACACAGCATA
TACACATACCTCACATACACACACCACCGTGTGCCTATGTGTTCACACGGTGGTGTGTGTATGTGAGGTATGTGTGTATGCTGTGTTAAGTGTG
TTCAGTGTGAGGTGTGCTTGTCTGTTCTGGGTACATGCACTGGCTCTATATACTCTATGTGGTATATGTGGGCTATATGTTATGATGTCTGTGT
GCTGTGTGTACCTCTGATGCGTGATGTCTTGATCGTCTGTGTACCATGTGGTATGTGTGCATGTGATATACGATATGTGTGATATGTGGAATAT
ATTACGATGTGGTGTGTGTGTGTGGTACATGCATGTGGTATGTGTGGGATATGTGTTTACAGTATGCTGTGTGCATGAGAGGTAGTTTGAAAAC
ACCGAGAGGTAAATGCTCTCTCCCCAGGTGAAAGGATTTCCTTGTCCAGCATATTCCAGACGACCAGCATTTTTTACTTAAACATGCTTTGAAC
ACTTTCTGGGTGGCGCTGTGTGTCTTTTCCATTCACAATTGTGTCCATGCATTCATTTCTCTTCTGCAGAGTGCGTTCCTCAGAGGTAGCATCT
CCCCATGGCAAGCTCCTCACACACCTCCACCTACTGACTGATCATCTGCTATGCAGGGGAGGTGGGCCGAACTGTCCAGGGCTCTGGATGTTTC
TCCTAGTGGCACCCAAGGCTGGAGAGGGCACTCTGGGTAGATGGCACTGGTTATAACTCAGGGAGCCACTTGCTGCTCTTTCCACCCCAGGCAG
GAGATGAGAAAGCCTGTGGTAACGAGGCCACACCTATGGCTCTCTGAGATGTGACTGCAATGCTTGTTTATGTGAAGTAGGCTCCACAGCCTTG
GGATGAAGCTCATCTGTAGGATACATTTCTAGCATTCCTGAAGTTCTTGCTTATACCCCAGTACCATATAAACCACATGTGGTAGTTCGTGCCT
CTGATCTCAAACTGGGGAGAGGGAGGCGGGGGAATTAGAAGTTGAAGGTCATCCTTGACTACATACTGACTGAGTTCAAGGTCATCTTGGGTAA
CTTAAAACCCAAGAGACTCAGGAGGGATGCATAGCTTTTCTTAGCCCGTTCTGTGCCTACTCCGGCCCACCGCCGGCTTTGAGTTCTCACTGGT
TCTGTTCCGTGGACCACTGTCTACCCTCAGGGTACTACGCCCTTGAGGACTGTGTCCAAAGACTACTGTGCACTTCTCTTTTTCTCAGTTTCTT
CTTAGAATTCCTTGTATTAACTAGAAAAAAAAAAAAGAATGATAGTGAATTGGGTCACCTTCGTCAAAAGCCGTGGGTTTGGTTAGCTAACCAC
AGGATGTGTGTTGGGAGACCGTGGGAATTAGGTAATAATAGAAAGTACCACACACCAAACACACACACCAGTGTAAGCACTCTAAGCTTGTATC
CCTGTGGAAACGGTTCTGGTAGTCATAGCCAGTGTCTCTTGTGGTTTGGGTTCTGCATTGTGACTCTCCCGTCTTTGTGGGCCCCAGGTACTTC
TCCATACACTTCCCAGAACTCTGAAGGGGTGCAGACATGGCGGGAAGTGCTGTGGACAGCGCCAACCACCTGACCTACTTTTTTGGCAACATCA
CCCGGGAAGAGGCTGAAGACTACCTGGTCCAGCGAGGCATGACCGATGGGCTCTACCTGCTACGCCAGAGCCGCAATTACCTGGGTGGTTTTGC
TTTGTCGGTGGCTCACAACAGGAAGGCACACCACTACACCATCGAGAGGGAACTTAATGGCACCTACGCCATCTCCGGGGGCAGGGCCCATGCC
AGCCCAGCAGACCTCTGCCATTACCACTCCCAGGAACCTGATGGCCTTATGTGCCTCCTTAAGAAGCCCTTCAACCGGCCCCCGGGAGTACAGC
CCAAGACCGGACCCTTTGAGGACCTGAAGGAGAACCTCATCAGGGAATATGTGAAACAGACCTGGAACCTTCAGGTGAGTCTGCTGGGCTTGAG
CCCACAGAGTTCTCTAAACATATTCAGAGCCTGTGCCTCCTTTACCCAAAGACTAGGATAGCAGATGCCTGTGTGCTGCTGCCCCTCGCTGCCT
CTCTTCCCACCCCATCTGCTCATGGGACCAAAGCGGAGTCCTCACATCCTCTCTGCCCTCCTCCTTTCACCACGAAGGAGAAGGTTGTGTTTTA
ACTTTATTCCTTTGAGTTTTTCTTCTTTGAAAGTCCAACTCTGGCTATTCATTACCAAGATCTGTTGTTCTCTCACTAGGAAGGAAAAGGGAAC
CAGCCTTCTCCACACTTAATCCATCTCCATGTTGTTATTTCCACCTTGTTTCTTCCTGGTCCAGGCTGCTGGCCAGGGCCTCCTCTGATGGACT
TCCTGCAGGAGTGTTGGGGTAGGAAAGATGGGACCGACGACATGGCTGAACCCTGATAGACATGAAGCCCCAGGTGCCCTCCTTACACATGTCA
TCCTTTTAAACTTTCTTTAGTTGCATGTACTGATTTCGTGTGTTGGAGGCGGGTGCCTACAGGTCTACAGCATGCATGCGGAGGTCAGACGACT
TGCAGGGGTCAGTCTCTCTTCCACCTCATGGAGTTCCGGGAGTCAAGCTGAGACGGCCAGGCTCAGCAGCCAGCATCTTTACTGCGGAGCTGTC
TCAGGACCCCCAACCTCACACACATCTTATAGCTCCTTGGCTGTTATGAAATTACCAGGTTTCACACTGAAACTAGTTTGAGTATAGAGCTTTG
GAGTATCTATTCTCTTAGCCCATTGGTTTATGATCTATATATTCAAATATTTGTATGGCTTTTCAAGGGCCCATCTCTTCATTCCGAAGCATAT
TCCTTTATTTATACACTTGATGACATGCAAGTAATATACAAAAGTCTTATTGGTTTTCTTATGAATGAGAGAAAGATGATCTAGAGGGAAGCAG
AGTTTGGTGTTGATAGCACCTGGGACAGATGGGTCTAATGTCTTTCTACCTGGCCAGCTCAGTCTCTCTCCATTCACTGCAGGGCCAGGCTCTG
GAGCAAGCCATCATCAGCCAGAAGCCCCAGCTGGAGAAGCTGATCGCCACCACGGCCCATGAGAAGATGCCCTGGTTCCATGGCAACATCTCCA
GAGATGAATCAGAGCAGACGGTCCTCATAGGGTCAAAGACCAATGGAAAATTCCTGTGAGTGACAAGCCATCTCCCTACCTCCTCACCCAGAGC
TAACAGCTTCCCCAGTTAGAACCCAGGTGGCCTGGGAAGGTGTCACACACCATCATCTACCAACACGGGAGTTGCTGTCACTTGTGACCCAGCA
TGTGTTTTATCTAGCGTGCCCGTAAAGATTGATACCGTGCCTAAAGAAAATGGTGTTCAGTCCCACGAGGGTTGGGTATGTTATCCGTGGTGAG
AGCAGAAGTTCTTTGGATTGGAGGTATTTTCTGATGGAGGAATATTTGCACACATGTAACGAGAAATCTTAGTATCTTATCTAAGCCTGCCCTA
GAAATTCATTTATGTTTTAGAAGCACTTGATGCTTATAATCTCTAGGCAATCAATTTCCTGTGTGTGTAGGTTTTCCTGCTAATGCATGTAAAT
GTATGTGCACCTCTGAATGGAGGTCAGAGGTGTACGTTAGGTATCTCTACAGTTGCTTTCTACCTTATATATCTTAGTATCTCCTTCTTATTTC
TTATTGTGCGTCTGCATGCTGGTGGGTGGGGAGTACTTGTGCCACAGTTGAGGTCAGAGGACAACGTTCATGGAGTCAGTTCTCCCCTTCCACC
TTTACATGGGTTCCAAGCATTGAATCCAGGTCACCAGGCTTGTGCGGCPACCACCTTTGTTCGCTGGCCCTCCCTTCCACCATGTATTTTGAGA
AACAGTCTCTCAAAGAAGCTGGGGTTTGCTGATTTGTCTATACTGACTGGTCAGCAGCCCTCCCCCCCCCCCCCAGGAATCTTCTGTCTGAACA
GAAATAGTTCTGTGGTCTTCAGTCTGGACAGCTCAATAATGTGAAGGCTTGGATCTTGGATTCTTGACCTGGGTATCTGCAGTGGGTCAGTGTG
AGCCTAAATTCAGAAGGTTCTGGAGCAAATTGGGCAGTGTAACTGAGCAGAGTAGTAGTCTGGGGTGCAGAGAGGCTGGGAGCTGTCCTTGTGT
GGTTAAGCCTTCTCATGTAACGATGCACCTGCCGAGGGAGTGCTGTAGGAATGCCTGCATTTGTCACCCCATGTCCCTACACAGAGGCCTTCTG
GGAACCACCACCCCATTCCTTCCAGTGAAACGTTCTTAGATTCAGGGGTGTGGCCTTACAGTCAGGCAACATGATCAGTATCAGGTTGTCTTCC
TAGAAATATAGGTGTTTCCTCTGCATTTGCATCTAGCTAAAGAATTTTCTCTTACTTCTGATATTAAAAAAAAAAATTTAAGGTGCTGAAAAGA
TGGCCCAGTAGCTAAGAGCACTGGCTGTTTTTCCAAAAGAGCCGAGTTCAATTCTCAGCACCCACGTGGCGGCTCACAACTGTCTCTAACTCCA
ATCCCAGGGGATCCAGTGCCCTCTTCTGGACTCTGCATGAGCTAGGAATTCATATGGTGCACAGACACATGTGCAGGCAAAGCACACAGAGACG
TAAAAAAAGAAAAGAGTAAAATGAAGATTATTTTTAGATTATCTTAATCATTTCTCTTTTTAAAAACCTTTTGTTGGTTCTTTGTGACTTTTAA
ATCATGCTCCCCAATCTCTCTTATCTCCCCCTTCCCTTGTACCCTCCCCCACCCTTTCAAACTCCCTGCCAACAGAGGAAAAAAAGATCTCATG
GAAGCTGTACTATCTCACAGTGTGTCCCAGACCATACCCTTTTCTTCACACTTCTGTGCTTGCAAATATTCATTGCAATGACTTGTTGGTCTTG
TACAAAGCCTCTGCCTTCTGCTACTCTCTTAACACTGCAACCTCACTGGGATTCCTCTCAGATATCCTGGTGTTGCCCTATGTCATGGAGATCC
TGTAGCTTTGCATCTATAGGACCAGCCCGGCAGTTCATAGATGGGGTACATGTTGGGATGGGCCATTCAAAGTCCCGAATCTGGGCAGGTATCT
TGTGTGTGGCTGTTCTGTGAGTATAATACAACATGCACAGGTCACTCTGTGTGCCATCTTCCATGCCACCCATGCTAAGACAGACCCGTGTCCT
CTCAGTCCTGTCATGAGGTACTCGGCAAGGTCTCTGTGTCACTTCTGTACACACCCCTGCTGGGTGTGAACTCGTGCACAGAGCACACCTCATC
CATCTCCACTATCCGTCTTGTTCTTAGTGCTCACTACAGCAAGGAGAAAGCTGAGATGGCCACACAGGAGATCTTGGTAGAAACAGAAGACTGG
ATCTGTTTATGGTCCAGTGTACAGCAAGTCCTCCATTTTGGGATGGGATGCACTCTTGAATCCCGTGTTAGTAGTAATGCCCTGCTGCCTTCTC
TCAGGTGTGAGCCCAGAAGGTTCTGGAAGCCTTCCCCTCTTCTCTTTCCTTCCTCACCCTCTGTTCATTTCCCACCGTAATTTCCCAGCTGCCT
GTGGAGGAATGACAGGGAAGACCAAGTCAACGGGAGTAGGCACCAAAGCTCCCTCCTCACTGAAAAGCAAGCACAGCCTACCTGCACTCACACG
GTTTGAAGTGCCACTTTGACAGGCCAGACTCTTTACCACATCCTTCATCCTCCGAGGCCCCTTCACACTGAGGAATTCAAGAGTGAATAAAACT
TTAGGCCTTCAGCCTGAGCCTACAGGGGCTGCTGACAGAAATGCACAAAGTATAGGAGTCTCTAGGGCAGCTGTGGAGGCAGCAGGCTACCATT
GTATTCCTCAGAGCTGTAAATTTCCTATTTGTGCTGTCATCAAGCCAGGAAGTCACTCAACTGCTGTGTGAGCCACAGTTCCTATTTTCTGACT
TGTGACATACATTGTGCAAAGAAGAAGAACAAAAAGGAACACAGAAGCAGTTATTCATTCAACAAGAGAGCCGTGTGTCTGAGTAGATAAGCAT
ACATCATCATGCTTTCTACGTCAGGCAAGATGCCACCCATCCAGGTTCACATGCTACAGAGCCTGTGACACGATAACATGCCACTTCCTGCCAT
GGTGTCTCATAAAATATGTGTTTATGTATATACATATTCATGAGCATCTATGTTCATGTATGCACGCATGTGTGTGWGTGTGTGTGTGTGTTAG
GGAGGGGTCAGAAGTCAACACTCAATGTCTCTCTATAACTTTCCATCCATTCTGGGTGTCTTGGGAGCAAGGACTGGCCTCTTGTCTTCAACAG
CTGTAATCTTAACTTGTATAGTGTTGCAACGGGAGGACCTCTCTGAGGACGTCCAAGGGGCTCTGAGAGAGAAAGAGGAGGTTAGCTGAGGAAG
GGCATGCCCTAGCCGAAGAAGTATCTGTGAGAGCCTTAGTGACCCAGTATTGCCAGAACATGTTGCATGTGACCAGCTATGGGAAGGGAGGTCC
CTATGATGTCGTGGCAACTGTGTTAAAACCCTTACTTTACTTAGTAATGGCTCCAGACGAAGACTAATGATGCCCAAGAGAAGCTGCGCTTATG
TGGTACTTAGTTCAGTACCCTCTAGTTTCAAGAATCTGCTGGGAGTCAAGTTAAGTGCTCCCTGTAGATCAATGTCTGCTTTCCTGTTCCACCA
TCCACAATGTTCCATTGTGAAGATTAACTGGGTCAAGTTCGGAGACAAGCTGGGGACTGTGGTAGACCTGTGCTCCCTACATCCACCGATGTTT
CCAATCAACTCTAAAGAACGCCTACCTTCCCCTTGTCCTTCAGAGGAAGTGTGGGCTCCTTACTGCAGCCTCTCCAGCCTGCTTAATGTCTGTT
GCCTCTCCTCCCCCATGTCACGTGGGCCTGTCACCATGTTTCTTCTCATATTAACTTTTGTTTGATCTCTGTCTAATCTGTCCTTTTTTTGCTC
ATTAACTTTTAAAAAATAGTTACATCTTTCAATGTCCAGAAATTAAATTTGGAGTAGGGTTTGTTTACTTTTCACAATTGTTCCTCGGCATTTT
TAGGAGCATCTCGTTGGTTGGTTGCTGATTCCTGTTTTGTTTCTTTAAGCGTTCCATGCATCATCGCTAGTTTGTATCCCATCTCATAGTTTTA
ATACTGAGCATATCCAGGTATACATATAATTTTCCTCCATGTCTCTCTCTTATCCGATGCCTCTTTTATCATAACTCATGTTTACTTACATCTG
GGGTGACCTTTTTGAGTTCATATTTGGTTGATACTCATCTGTGGGGATGCAGAATGCCCTGTGCTCAAGTGCTTTCCTTCAGGATTCAAGTGGC
GGCTGCTACCCTAAAGGCTGGCTCCTGCCCACTGTAGTGCTGGTAACTGTGTTTGTCCTCAACTCCACTAACACTGGTGTGTGTGTGTGTGTGT
GTGTGTCTGTGTGTGTGTACATGCACATGCAAATGTATGCAGGTGTACATGATAGTGCATACATACAGAGGGTAGAGGTCAACCTTATATGTTA
CTTCTCAAGAGCCAACCACATTCCTCCTTCAAAAAGTCTCTCTCACTAGGACCTGGGGCTCATCAATTGGGCTAGGTTTCCTGGCCAGTGAGTC
TCAGGGATCTGTCTCTCTCCACCTCCCTAGTGCTGGGATTTAAAGGATATGCCCCCTGCCCCACCCTCGCTTCCATCTCCTCCCACTTGCTCAG
CAAGCATTTTACTGACTGGACTATCTCCTCCTCAGCCCCAAGCTCGTGCTGTTGCTTTCATTTGCCAAACAAGTTTGCCTCCTCAAGTTCCAGC
TTACGGGGTGATCTATGCCTACTTTTCAAAATTATATTGTGGGCATCAAAGAAACCCTTCTCTTTGTTCGTTAAGGAATGAGACAGTAACTGTT
GTGAGCCTCCTGGGCCACGGCGAAAGCCGAGTTCAGTCAGTCAGTCAGTCTGTCTTCCCACACTGGTATGGGTGATCAAAGCAGGTCATTCATT
TGATCTGTGTGGGTTTGCCACCAGGGCGATCAAGAAGCATCCTAGCCCGTCTCTGAAGGGAAAGTTCAAAGACAGCCGTGGACTGATGTCAGTG
TCAGTTTACTGATGCTGCTCAGAGGCCCTGGGAAGCTGGAAGAGAAGCCACAAAAAAGAATCTCTACCTAGGGACTGGAGCAGAAAGACAAAGA
GAAACCAGCGAGGGGCTGGGTGTATGCCTGCCTATCTTGTACAGACCAGCTGTCAGCCTCAGGAGATAGTCCCCCTGGGAGCTGTCCACCCATG
AGATAGTCCCCGGGAGCTGTCCACCGAGTTCACCCTGGAACCTGCGGCTTGTTGATTATGCTAGGACATCTACTACCTGCAAGTCCCAGAGATC
CACCTGCCTCTGCCTCTGCCTCTGCCTTCCCAGCACAGGGACTGTAAGCAAAATCATGACACTTGGCTGTGTTTGTTTGCTGTTTGTTGTTTGG
GGAGCTTGCTAACTGGCTTGCTTGTTTGTTTTTGTTTTTCTTTGGTGGTGGTGGTGGGGGAGGGTTTCAAGACAGGGTTTCTCTGTGAAGCCCT
GGCTGTCCTGGAACTCACTCTGTAGACCAGGCTGGCCTTGAACTCAGAGATCCGCCTGCCTCTGCCTCCCGAGTGCTGGGATCAAAGGCGTGCG
CCACCACTGCTTGGCTTGTTTGTTTGCTTTAATAGGGATTCTAAAGGTTGAACTCAGATCATCCTGGTTGCAAGACAAGCACTTTTCCTACTGC
ACCCTCTCCACAGTGCAGGGAGAGATGATTGTGGAAGATGTTTCAGAAGACGCATGGGTAGAGAACACAAGTTTGGCATGCCTCAGATCTGGTT
TGCTTTAGGGACCGATCTTGCTTTCAGGATCTGCACAGACAACACCCAGTGTTGCTACTTCAAGGTGTTGGCAGGCACAGCGTTTCTGTACCAA
ATGTCACTTATTCCAGGATCCCTGTCTCCTGTTTTCCTAGCCTTCTTGGAAAGGCTACTGTGCATTCTTAGCTCTGAGCAATGATCATTTTCCC
ACCTATTATTCCCCTCTCTCTTCACCTCTCTTTCCTTCCCCTCCTCTTCCCCCGCCCATCTTCCATCCTCTCTCCTCCTGCCCATTCAGACATT
TTCCCTCCCCTTTCAACTCTGACCCCATCACAGATGCCCCTGGACGATAGCCACAGAGACTGAGTGGTACTCAGGCTGGAGTGAACCTGCTGTC
CTGCTCTCTCTGTATCAGATGCAGACCCCATCCCTAGAGTTTTACACACCGAAGCCTTCAGTATCTCATTATCTCCCAGGCCCTCCACTCACTG
CCTGGTCTCACCACCACCCACACATTCTTTTACCCTGGAGGGCACACAAAGGTAGAGAAGAGGGTTAAGCCCTTCCAGCAAAAGTCACACCCTC
CTCTCTCACATCTATGAGGGGCAAGTTCAAGGTCTGTCTGCCGAGCATGCACGTACATACACATACACATTTCCTCCTACTACTTACCGAACTT
CCCTCTCCTTACCGCTCCTCTTACCCTCCCCCTTACCCTCCCTCTCCTCCTCCTCTCTCTCTCCCCTTACCTTCCCACTTCCCCTTCTTCCCCC
TCCTTTCTTTCTCTCTCTCTCTCTCTCTCTCTCTCTCTCTCTCTCTCTCTCTCTCTCTCTCTCTCTGTATTCTATGCCTACTTATGTGTGTTGA
TGTGCATGCCCATGCATGCAAATGCAGATGCCCAAGGAAGATTTCCAGAATCTTCCTCTATCACTTCCCACTTATTCTTCTGAAACAGGGTCTC
TCTCTGAGCAGAGCCTCACTGCCTCAGCTGGGCTGGTCAGCCTGGGAGCTCATAGCATTTGTCTCTCTCCAACCTCCGTCGCTGGGCTTACAGG
CACATGTGAAGATGTCCAGCCTGATTTACCGCAAGTGTTCTCACCCACTGAACCATATCCCAGCCCCTTACACGTGTGTGTGTGTGTGTGTGTG
TGTGTGTTTTCACACATTGTCCTTTCTGCAAGCCAGCACACATTGTGAATGGTTTCCCTAGAAAACAGGCTCAACCCTTTATTCATATATGGTG
TTTTATTGTGTGACTGGGCGTTACCCAATGACCAACCAGGCTTTTATCAGCCACATAGTCATCCCAAAGGAGTTGGCACCTCAGGGTGCTCGTG
ACTGACTGTAGGAGGGAGGAGGTGTTTCCACAGTGGTGAAATTTCAAAACACAAATCTACGATGGCGGAGAAGGGTCTTTCACCCTTCACCAGC
TTCTCTGAGCTTCAAGCAGGGGCCTGGAGGGAGCCCTCGCCACTCTGCCAGGCAAGCGCCATTTCAAAAGAAATCATTCGTAAAGTTGCAACCT
CCTCCTACTTTGAGGAGATGGGTATGGCAGGAGAAAGGGAAGTGAGGTCACTTCCGGTCCCAAGGCCCAGCTCAGCCTGGACTGTGGCTGTGGC
CTTGATGTTGTGTGCTTGGAGAAAGCTGTACAAACCACCCCAGTTTACAGACACCCTTCTCCAGGCTGCTGGGACTTGATTGAGCTCTTCCCAA
GTCCAGTGCATTTATGCTGGATGCTCTGAAAATTCATCTTCCTTCCTTTAGCAAATGCAGAGGCCACAGAGGCCTCAGGGACTCCCAGACCGCA
GATGTCCCACGGACTCGCCCTTCCCCACATCTCATGATTAGAACGCATGAGTTGCTGGAAAGGCGGCGAATAGCATGGCCTCAGAGGAATGAGG
CTCTGAACTGGGAGAGTCACACCTCCTATTGCCATGCCAGCCAGATCTCACCTTCTCCGGGGCCTTCCTAAGTACTGATTCCTCCTATGATGTC
AGGACTGCCTTGTGCCTGTGTCTCGAGGTTCAAGGGTGCACCTGTATGTTGTCACTCGCCATGGGGGCCTGAAGTCCTACATGTAGCTCCTGGT
ACCAGTGGAACAATGAAGTCTCATTCACCTACAGACGCCTCAGGCCTTGGGCTGTCTCTGTTGGCPCATGCCACGTGGCTATCATTCCCGTCTC
AGGTGGGGGATGCTACCTGCTAATGTCAAACAATGTCTTAAACCCGGCCTTGGCACTGAAGAGAAAGGGAAGAAGACCATAGGAGTAGAAGAGT
TCATAACCTCCTTTGTGTGCTCCAGAAAGTTCTGTTAACAAGCTCATTAGACTCCTCACCTGTCCTTCACCTGAGTCTAGGGGCTTCACAGACA
CCCCAAAGCCTGTGCCTGTGCCCCTGAGCCACACCCCCCCAAGCTCTTTCACTGATCTCCAAAGCGCCATGGCCCTGCTGGATCTTTGCCATAG
TCCATCTCCAACCCTTGGCGTTTCCCTCTGGCTAACTCGCTGGCTCTGGTACAGAGCCCTCCTGTCATCTCGAGGTCTGGGTGCTGCGTGCCCA
CCACCTGCCCATTTGCTGGTGCTGGAGAGCACCTGCTGCCCACCAGGGCCACCAACTGTAGTGACGGTACTGCTTCCTAGGTCAAAAGGTCGCA
GTAGGGGTAATATGGTGTGTGCTCTCTCTCGCAGGATCAGGGCCAGAGACAACAGCGGCTCCTATGCTCTGTGCCTGCTGCACGAAGGGAAAGT
ATTGCACTACCGCATTGACAGGGACAAGACCGGGAAGCTCTCCATTCCTGAGGGGAAGAAGTTTGACACCCTCTGGCAGGTATGTCTCTGTCTC
TCTGTCTCTCTCTCTCTCCACTCACTTCTGTGTGTCTCTGTATCTCTCTCTCTTTGTCTGTCTGCCTCTCTCTCTGTCTCTCTAGACTCTTTCT
TTCTCTGGACTCTCTTCTCTGTCTCTCCCTCTCTGTCTCTGTCTCTCTCTGTCTCTCCCTCCCTCCTGGAGGGCACAATGCAGACCCACTGGGA
TGCTAGTTCCAAAACTCTTAGGAACAAGAGGAGGAACGAGGCTATCTGTCCCATACCCTCCATCAACCCACTTTCTGATTTCTAGAAGACTTGT
GTGAGAAATGTTCACACAGACCGTCATCAAGTCAATGAAGCCACAAACAGGCTCCGTCACCTGGTTAGAGCCATAAGTTCCTGGTGCCTCAAGA
TCATGTATTTTAGGGCAGCATTAAGAATTTGACAGAAGGCCATGCCATCAGGGACTGGAATAACATGTGCATGCATGCACACACACACACACAC
ACACACACACACACACACACACACACACACACATACTGTAACTGATCAAACAAAAACCCTAGGGACTGTGACCTAGCTGGTCCTGGAGCTCTGA
AATGCCAGGGTAGACACTGAATTTCAGGTGACATCCCAGTGGATAACCAATATGGGAAGCACCCTCCTAGGCAAGCATCCTCATAGCCGAGCCA
CCACAGGAACTTAAAACAGCTTAGAAAGCAAGTCTACTCTTCCTGGGCTGCAGACAGCTGCCTGTCCAAAGAGCACCCCATACCTGCTCTGCAA
TGTGTTCTCAACCTAGCTGTCAGTAAGCCTCAAGGCAACTCAAGCACCCCAACATTATTCACCCAGCTCCCCACATTTGCTACTGAGCACATTC
CGCTTCCTTAATTTGATATCATAACTACATTAACTGTGGATGCAGAAGGATCAAAAGTAGTAAATTCAAGGCTAGCCTGGGCTACATAGAAAAA
ATCCTATCTCAGCAAATAAACAAAAAATTAAGTCAATAAAGGACTAAGTGATGGAGATGCTATGGAGACCCCGAACCCCTAATGTATCCCTATC
TGGCTACGTGATGGTTATTTTTTTTAAACTATTTTAAAATTTTATTATTATTATTATTATTATTATTATTATTATTATTATTATTATACATAAG
TACACTCTAGCTAACTTCAGACACACCGGAAGAAGGCGTCAGATCTCATTATGGGTGGTTGTGAGCCACCATGTGGTTGCTGGGATTTGAACTC
AGTACCTTCAGAAGAGCAGTCAGTGCTCTTACCCACTGAGCCATCTCACCTGCCCGTGATGGTTAGTTTTAGTTGTTAATTTGATACCATCTAA
ATCACCTGAGAAGAGAGCCTCAGTGAAGGATTGTGTAGATTAGAGAGTGAGCATATCCATGGGGATTCTTTTGATTACATTAATTGATGTGGGA
AGACCCAGGTCATTGTTGGGTGGCACCATTCCCTAAGCTTGAGTTCTGGTCTGTGTAAGAGCAGAGAGTGCTCACTAAGCAGTAAGCATGCATG
CATTCATTCACTCTCCACTCTTGACTATGCATGTGACCAGCTGCTTCAAGGCTAAAAAGTTTGTTGACTCTATCCAAGGACATCTTTGGCCCAT
CCCCCGACTACCCTGTGGAAAAGAGACTATCTGGGGTTTAGATGGTTTTAAAACCTGTGAGCGCTGTGACTTATCCGCTGTCTCCCCCTCTCTC
TCTCTTAACCATTTTCTTCCCTGTTTCTTTTCCAGCTAGTGGAACATTACTCTTACAAGCCAGATGGGCTACTAAGAGTCCTCACGGTACCATG
CCAAAAGATTGGTGCACAGATGGGTGAGTGATCCGCACAGAAGAGGCTCTGTTTTGGGGGAGCTAGCCATTGTCAACCTCGTGTCAGATTTGCC
AAACTCACACAGGGTGAGGCTTCGGCTTTACCGTGTGATTAAACTAGATGTCACTCAAATTCTTGGAATCATTAGCTGAAAGATAACCATAACA
AAAACAAACAAACAAACAAACAAAAACTAGAAGATTTTGATTGCATGTGTGGCCAGGATGTTGGCACATATCTTCAGTCCCAGCATTAAAAAAG
CAGGGTAGGGAATCTGTGAGTTCAAGGCCACTGTCAGACCCTGTCTCAAAGAGAAAAGCTTACTTGGTGGAATGTGTGAAAAATCCAGTAAAAG
AGAAAAGTACAGATGCAGAAAGTTGAGTCTCCCCAGTATGTTCCCATCATACTGATAGATCTGAGAAGTAGATGGTTTGAACAAACACAGAAGT
ACACAGAACTGTAACCATGACATATTCTCCTTCCAGGCCACCCAGGAAGCCCAAATGCCCATCCCGTGGTAAGTATTCTCTGAGGCGTCTCTGC
TGGGGAAGCAGCTGTGTCTGGACACATGGGAAGGTCAGCCTTTAGTCAGCACGGGATGGATACTCTGAGCACATCCCGGAAGATCTGGGAATCC
AGTGAGTCTGACAAAGCCTCACCCTGTGACCACCGCCCAGCTAGACGTGTTTTCATGGTTTCTCGGCCGTTTTATTTTCTCTTCATTCTTTGGA
AGTATACAGAGTGAGACAGAAACCCCCTAGGAGATCAGAAAATCCACCGGTCAGATGTAGAAAAGGGGAATATATCGTCCACGTTCAGTCTTGT
ACTGTCTGTGTGAACTCGTGTGTGCCATGCACAGGTCTGCCAACATGCATATCAGTATAACATGGCCAGCGGATAGGTATCTCCTTGAGGTTGG
CTACCCAACTTCTTTGGCCCAACCAATTGACACCTTATTACTAGGGACTTGACTTAAATTGATTTGACTAATGATTAGGGGAGGCAGGGAGGGG
CCACTTGGCAAGATTTCAAGAGAAAGGTCCAGAGAAGGAAGGAGTCTTGGCCAGAATGCACATTCCACTCCATGCCACTCACTAGATAAGAACG
GAGCCCAGATGGCCCTGTGTTGGGTTAACCCTTTCATTGCCATGCCTTCAAAGAGAGAAGAACCTCAAGTCCCCTCTCCATCTGGGATGTCGGA
GCTGGTACTTAGGTTACTTCACAAGAGCATCTCTTCTCATATCTAGGGTCTTCAGCGAGCACATCCCAGCTCATTAGCACCACCAGGGAATAAT
TCTTGACTTCTATCTTGCTAGATGCTAAAAAACCCAATATCTACCAGGCACATGTTCTGTCTCCAACAGGGGCGGTTGGCCACTTGCTACTGAA
CCTGTTCATTCCAGGGTTAGTACTTCTTACCAGACTGAGAGCCCTTGAGTTGGCTAACACACCCTGGAGCTTTAGGGTCCTCTCAACCACAGTG
CCCCCTGGTGGCCTCACCTGGTGTTTATCATTCTGTGCCCTTGAGACAGATCTGTCTAACTTGTGACCCGAGGGTTGCGTATGTCCCAGGATTG
TGTAAATGAAAATGTTATATTGCAACCACTGACACCACTGAACAGCCAGTCACTAGAGTCTCTTGTGGAGTCTGAGACACAAGATTTCCACAGA
CAACGTGGCTGCTTCTTGTCTCAGCTTTAATTTTGTAGCCTTCAGTGGGAAATTATTTATTGAAAATTCTTGACCTCTCTGCCAGAATGATAGG
CTCTTCTGGAAGCTTCTCAAATGATCCGGACTTGCATGCAGGAAATCTCCCTGGGGCAGGTGAAGGGAGCGGGCAGGGCATTTGCAGAATCGGG
GCCAGGTAGCATGTCATGACGAAGAATATCCTTGCATGTGTTTTTCTGAAACTAAACGCTCTATTCCTCTCTGCCGTTGTGGTTTCTAGACTTG
GTCACCGGGTGGAATAATCTCAAGGATCAAATCCTACTCCTTCCCAAAGCCTGGCCACAAAAAGGTGCTAAAGCGTCCCGTGCTTGCTGTCCCT
GACTGAGTGGTAGGATAGACCCTCCTGCAGCCCGTGCTTGGCTGGCTATCCTGTACCCAATGCGGCTTTGCCTCTGTGCCTCCCGCCTGCCCAG
CTCCGAGCATACCCCCTGAGCCTCGCAGCCCCATGCTTTGTCTCCTTACCTCTCTTTACACCCTGCCTCCTGTGCTCCTACTGAGCCTTGCAGT
CCCTGCACAGTGCACACATGCCCTCCCTGCCTGTGGCTTCTCCGTCACCGCCGCTGCGCAGCCTGTCCACGTTGCCTTGCCCTTTACAACACGT
ACCTCCAGTCACTTCAGTGAGCCCCACCTGCTGGCTCTCATTCCAGAGAGTCTTTGCACGCTCAACTCACCTTCAGTGTCCAGAGTCACAAGGG
GCAGCATCAGGGCTTCTGCCACATAGAGTTCTTCCCAGTGGAGGCAACCAAGGTCCCCAGGGGAGCTCTACCCCCTGAGACAGCAGGTTGATTT
CTGCATTTTGTTACATGGCTGAATTTTGAGGGACAGTAAAAAAAGGCCTTCATGGGAAGATGGCCCAGAGGCCAAAGGCAGTTGTCACCAAGCC
TGACAGCCTGAGTGTGATTTCCTGCAACTCACACAGGGTGGAAGGGGAAAGCCAGAACTGACTCCTGCGAGTTGCCTCTGATCCCCATGTATGG
GCCTTGACTCCTACACAGATTTATTTTCAGGGTTTAGGAATCAAACTCAAGCGCTTCACCACTGAGCTGTGCCCAAGCTCCTGGTTTCTGTTTG
TTTATTTGTTTGTTTGGTTGGTTAGTCTGTGAGACGGGATTTTACTCTGTAGCCAAGGTTGTCTTTGAACTTAACTCCACCTCCCAAGCTCCAG
ACTTATAAGAAGTCCTACCCTGACCTTTGTGTTGACTTGTTTATAGTGAATTACATACACAAGCATGCACACACAGACACACACACAGACACAC
ACACACACACACACACACACTCACACGCACGCATGCACCCCCATATGTGTATATGTATGTATATGCACAGATATATGTGCATATATATACACAT
ATGTAATGTGTATTATATACATCTGTGTATATAATATGTATAGGTTGTTCTATCATATTTATTTTTATCTTCTTTAGGTAAAGATTAACTTTAT
TAATAGTTGTTTCAATTTTATATCAATGTTTTTTCCAAGCGTGTGGGCATATGATTAAGCCAGTGTACACAGAGTTGTATATTAAGGTATTTAT
TAGCAACAGACAGCATAAATGCCCACGAGTTCTACTCAGCAACTATTTGCTCTGTTGGCCACTTCCTAGAGGGTCCTAACCCTGATGGCCAGAG
CACAGCGGACAAAGGAGCATGATGCTTGAACAGGAAGGCAGCCATGTTGTGCTAAGCTCAGTCAGTGACGAGAATGTTAGAGGATAGTCTGCGG
CTGCCTGGACCTTTCTCAGAGCCCGACACACCCCCATGTGATTGGGGTAGCTCATGGCCAGCAAGCCCAGTGAGAGCCAGGCAACACGGTACCT
CACAGGCTAAGGGACTTAAACGTGCCTGATGAAGGAAGCTCTTCAGAGGTGTCCACTCAGGCCAGCAGCCACTCACAGACTCTGTAATCCTGAA
TGTGACACCACACTTAACAAGCCTCACAGACACTGCAAGCCTTGTGTCTCCCCCTCCTCAACCGACTCTTCAAGAGCTTCTGATGACACCCTGT
CATTCCACTTGCCGTAGCCCAAGGCTTGTTGCTAACTCACAGCCCCCAGCTCTGCACAGTCCTGGCAAGGAGTGCTTTCATCCCAGCATCTCAA
GTTTATCTTTTGGGTTCCCCCGGAGACATATCAACCAGAGATCCTGTATGTACATGAGCACACATACACATGTATGACACACTCATATATACAC
TCATACATGGAAATATCTGTGCCCGTGTGTACATAGTCGTATACCATATTTGAAATGCATATGCCCTGAATATGCACCATATACACACATATGC
ATCTGCATAGTATGTGTATTTGTGTATGCATCTATTGCTGCTTACACATGGCAAATGGATTGCCTGCGGATAGCTACACAAAGATGTATTTCAA
GGACTTACAGAGTTAAACAGACCAGTTAAGTGATTGCAATGTCCATGGGAGAGATTGTCAAGCAGAGGGGATCCAGAGCCAGGTGGAACCTCAG
TGGTCTTGCTAAAGAAAGCTGCTGCATCGTAGGGGTCAGCTTCCCTTCCCCGCCCCCTTTTCCCTTTCCTTAGAAAGTCTAAACTTACATCTAA
GAATGCTCACCTGAGTCAGGCCCATGTGAGATCATCCTCATGATGGAAGTCACCTACTAAGGACTGTAAGCCCATCAGTGAGCCCCTTCTCAGC
AGCAGGACTGCCTCAGGCTGGGTCTTCTGCGTGACTCAGTACTCACCACTCACGAGAGCCACTTGTCATAGCGAAAGCCAACGCTGCAGCTGGA
AGAGATAGCGCAGTAGGCAAAGTGCTCGTCACCCAAGCACGAGCCCCTCAGTTCAGATGCCCAGCACCCAAGTAAGAAGCCAGGCACAGTGGCA
CACGGTGCCATCACAGTGCTGAGGACCTGAGGCAGGAGGGCCCCTCAGCCAGCCAGTCTCGTCAAAGAGGCAAGCTCCAGGTTCAGTGAGAGAC
CTTGCCTCAAACAATAAGGTGGTGAAGGCATTGAAAAAGATATCGGATGTCAGCCTCTGGCCCCCGAATGTCCACATAAGCAATCGCACGTACA
TGCACATACATGTGCAGGCACATAGAAATCCACCTACGCCATGTGCACATTTTTAGAGAAGCCGACACCAACAAAGCCCAGTCAACAAGCACTT
GAAATAACTCTAGTGTCTAGAGAGTTAATGAAAACTGTGCTTTGATCTTAGGATTATCAATTTAAAATGAAACCAGGGCGGGGACTTAGCTTAG
TGACAGAGTATTTGTCTAACAACCATGAGGCTCTAGATTTGGTCCTTAGCACCAGGAGGAGAAAAATACTTGGAAAGTCAAATACAGATTTTTG
AAATAATGTATTTCTTTTTATCTTATCTGCATTGGTGTTTTGCATGCATGTGTGCCTGTATGACGGTATCCGATCTTGCAGTTACAGACAGTTG
TAAGCTGCTATGTGGTGAGATGGCTCAACAGTTAAGAGCCCCGATTGCTCTTCCAGAGGTCCTGAGTTCAATTCCCAGCAACCACATGGTGGCT
CACAGCCATCTGTAATGGGATCTGATGCCTTCTTCTGGTGTGTCTGAAGAGAGAATGACAGTGTAAATAAATAAATTAACTTTTTTAAAAAAAT
AAAAAGAGGAGGAAGAGAAAGAGGAGGAAGAGGCCAGGTTTTAGGTCTTTGTTGTGAGATCAAAGAATAATTATACAGGGCATGTGCTCATCTT
TAGGTGAACCTAACCGCATCCCACCTTCTGACAGAGTCTGCCCACATCACACTGCCTGTGCTCCTTCTGAGACTGGAGACTGTAGCCAGCTGAT
TGCAGCCAGTGTCCTGGAGCCCTATGGCTTCTGGCCTCTGGGGAGGGPTACCACCCACACTGCCACCACCGTTGCCTAGTTTGAAAATACTGGC
AGGAAGCTGGGAGTGAAAGTCAAGGTCCAGGAAGCAAAAGGCAAGATTGTGGAAGCAGGACACAGGGCAGAGGCTACCTGCTCCACAGGACAGA
GGGTGTGCTGCAGAGCTCTGGTGATCACGGGCCTAGAGAGAGAGACAGGCAAGTCCCTGGTGCCTACTGACCAGCCATTCAGCCAGTGGAGACC
CCGTGTCAAACGCTAGGGTGGATAGTTCCTGAAAGTTGACCTCTGTCCTTCACAGGCACATGCACACACACAACTGCTCACACACACACGCACA
CACAATATCATCCACGCACGCACACAAGCCTGTCTAATGCATCCCCACTTTTAACACACTGTGACAGAGTAGTTCGAACTTGAAAATTATCTAC
CCAAGGCTGGATTCCTGGCGCCATCTAGGGGCCACTAGAAGTTGTGCATAAGTGGAAAGGGTAAGGGTGAAGCTGTCAGCGCTTCCTTTACAGC
TCTGTTCTCAGTGCAGAACAGAGATGCCAGGGAGGGCCAGACCACCAAGTACAAAGTGGAGAGATGGATAACGCATGCTAGCGCTGTCTACCAG
CAATGCTTCTCACGACAGCTCACATTTCCCTCCCTTCATCTTTCAACTGAAGCTTTGCTTCCAGAGCTTTCAGTGGTGACAGTGGAGCTGGTAG
TGGAGGGGAGGAGGGTTGTATCTTCATCTGCACCACAAAGCAGCCAATGACTGCTCTGGCCCTGCCCATTCCTTCTGCAGGACACTCAAGCCAG
GTGCCATGATTGTCCCAGTTCCCTAGGCTGCCACCTAGGTGCAGGAGGCAGGAGGTGGTGGGTCCTAAGGGTGAGGCATCCAGATGAGAAGGGT
TATGAATCTCTTGGGTCTCAGGGGCTCTGTCTGGATCTTGAATGCTTGGACTCATAGGGTGCTTTCTGTTGGGCAGCATGGAAATGAAGCACTG
AGCTACATCCACTTAAAGAAGAATCCCAGTTTCCAAGGGAGGTGCCTGTTTTGTCCTTTTGTCTGTCATCGAGACATAGGGGTAGCTCCTGTCT
ATCTGGGTGTGAACTCCTTCCACGAGCTGGGATGCTCAGGGGTCCCTCCCCCCACTGATCCCTACCTCCTTTTGATATACTGCCATTTTTTAAA
ACTGATTTCCTAAGACCCTCAGCTCTGCTTTAAAAATATTCCAAGAGATTTTGTTAGGAAATTGTGAGTGCTGGTTTATTATAACAAGTCTTTT
GAAGCCCCCTTGCTCCGGAGATAATTCCAGAGAGACTGCATGAAGCCCCCAGTTGAGACACTGAGCGGCAGAGGGTAGACGCTCTTCTATCCAC
GGGAAGGATTACGCCTGTCCTCCCTACCCTGGGGTGTCCTTTAACTTCATGATGGTCAGATTGTCTTGGGGATCTCAGACATTTCTTGTGTACA
GAGGAAAAAAATCAAAAGACTTAACATGGTAGTCACGCTAACCGGAAAAGACATCTGGAGACTGCAGCTGGATGATGGTAGCTATTATCAAGTA
TAAACTAGGGTAGTTCTTCCTAGGCAGGGCTTCTAAGCAACAGCCAACTCTGCTGAAGTGATGTGTAAATGTTTAAGAGGGTCGCTCGCAAGAG
ATTGCCTATGCTTAGTTCCCTTCCTGAGAGCAAAGACTGAAGAGAGAATTCATCTCAAACCATGTGAGTTTACATTCCACATCTCCATCTTCTG
AAAGGTCTCTCTGTTCAACCATCCACCATCTATCCACCCACGCATCCTTCTGTCCATCTACCAACTCACTGATTCAACCGTCCATCCATTTACC
TATCTATCCACTCAGCCATCCATTTATCCATCCATCCACATATCTGTCCACATACCCATACATCCATCTATGGATCTGTCCGTACATACACACA
CCCATTCACCCACCTATGTGTCCATCATCCATCCTTTCACACGTCTGTGTTGTGTGTTTCTCCTTAGCATCCGTTACCAGCGTGGCCCAGCTAC
TGAGCTTTGATTTAATGCCTAAGTCTGCATAACTTCTGCTCTGTTGCCATGGTTAGTTCTATCACACAGTCCTTCGGGAGTGTTGAGAGAGGCC
AGGAGAATATGGATCCTGTGACATCTGAGACTGGATGTCCTTTGGCTTTGCTGAAAGAGGTCTTTCCCACAGGTGCTGGCAGACTCTAACTCAT
GGTTAACACCACGAAGCCAGCAAGTCGTAGTGACCGACTGTGACCCAGCTGCTAAACATTGATGGTGCGGTTGGTCACGTCAGTGAGGCGGGTG
GGATTTTTGATATGTCCTTCAAGATGGTGGCTGTGTAATGTTAGCTGAATGTCTCTGTTTCTCTACACTTCCCCTCAAATCTGGCATTATCTCA
CTACTTCATGGCAATGTGTGGATGCTGATAAGGTAAGTAGATATTCAAGACAAGACAGAAATGGGAGGAGAGAGCCGTCAGAGCTGGACGGCGG
TTTGCCTGTGAGATGGGTTTAATAGCCACCACTTTGCTTTAATTTGTGAGTAAAGATGGGCCAGAGAAAAGTTTTTAAAAATGTATCAAATAGA
ACTATAAGGAGATTCACCCCCTCTGCCTACTTCATGAAACAAATTGATATTGATGGATCGAATGAGAAATCTCCGTGTATAAGGGCCAGTGTGC
CTTAGTGAACAGCCACAGGAAAACCAGAAGAGTTCTCTTCCTTCAGGAAGACAGAGAGACTGTCTCTTTGACAGGGCATGTGCCCGATGACATT
ATCATGGTCCTTATCACGATAATCACCGAAGTGCATGGGGTGGGGGTGAGGCAGGATACAGCCTTCCTCTCTGAGACTTGCAGCTGGTTGGCCT
GGGCTACCCTGTGGTAGCAATGCCTTGGTGTCTCAATCTGTATGGAAGGCAAAACAGGTGGACACGGTTTGACTTTTTAAATAACTTCTGCCTG
ACGTTAGATGAAAGCAGAAATGAACCCATGGGGAGGGAGCCTGCGAGGTCCTGCTATTTTCTGGGAGGTGTGAAAGGAAGTTCTCTTGGGATAG
TCAGAACAGATGCTCTTCCCTCCNNNNNNNNNNNNNNNNNNNNCCCTCCCTCCCTCCTTAAGGCCACTTCAGGGGTGGGAGCTATAAGCCATGA
GCCTGTCTAGCCTTCTCAGGTCCAAACAGTTGCCTGAAGAATGAGATGAGCCTTTCTGACCTCTTCCTAATCCTCTGCCTCCCCTGCTTATGTT
ATCTGTGAACACTTGGAAATGGTGGCATCGGGGCAGAAAAATAACATCTTCTCATCTGTGTGCTTCAGCCTGCCCCACCCCAAGGGAGCCGTCC
AGAGAGCACTGTGTCCTTCAACCCCTATGAGCCAACGGGAGGGCCCTGGGGCCCAGACAGAGGTGAGAGATGAAAGATGCACTTTTACTCAATT
AAACATTCGAGCAGGGTACACACAGGCTTGCCAGCATGTAGAGGCAGCTTACAAATTCCTGGCACCATTCCAACTTTGCCTTACAGTGATGGTC
CACACTAAACAGTGGTGATATCCACCAGCCACAGGTAGAAAATATCACCTATCCATAAAACAAGCAGTCCTGTCTAGTGAAAATGGTCTTTTAT
AATTAAAAGAAGCAAGCAAGCAAACAAACAAAAACCCTGAGTTGTTAGCATCGGCAGCATCATGCTGTGTGTCTGGCACATTCCGGGGACCCTG
GTTGCCATTCTTGGAAGAAAATGTTGCCAAAGCATAACTGTAACCTCTGGGTGGCTTTGACGGTAGGCACAACTCACAGATTCTTCTAGATTTG
TTCAGGGAGCTGTGATGACTGGGAGGCCACAGGG0ACACAGTGCAGGAGGGCGCAGGAGACTTGTTCCCAGAAATGGGTTTTTTCGGTACCTTT
CGTAGGTTCATGAGTGTCTGCATCTGATTTTCTACTAACAAGATTGGGGTTTGTTTGTTGCTATAGTTCGCCTCCGAATAGAGGCTTAAAGCAA
CAAGAGTCAGTTATCTCTCATTTCATTTATGCCTTGTGGCTGGAGGTGTGACTCATATAGGGTGCTTGTCTAGCATGCACGGAGCCCCGGGTTT
CATCCCCAGACCCGCAGAAGCCATGCATCGTGTTGTATACACCGTCGGCATCATTGCTGGCTTTGTGTTGAGTTCACGGCCTGTCTCACATGAG
CCTCAGAGCAGTAGAACTCCTTAGCATCTTAGAAATGAAGCATGTTGGCTTTTTCAAGCCTTGAGAAGCACACATATCATTTCTCTTGAGCTCT
GTTGGTTCAGTTATGAAAAGCCAACAAGGACTGTGTCGTTGGTGCTACTTCTCAATAGGAGACTGTCACAGCTGGCTTAAAGTCTAAGGAAGGT
TTTATCTGCCATTAGTACTGGTGTAGAAGAACCATCTTGTCTCTTCCCTTGCTTCGCTTCTCCGCTGCATGCAACTGTCCCTTAGACGGAAGGA
GTTTAGGGCAGGGTGGCAAGGGTTGTAATGTCACAGGAGTCCAGACTGGAGTCATCTGTTCCCTGACTACGTTGCAGGCCTTCAGAGAGAAGCC
CTGCCCATGGACACAGAGGTGTACGAGAGCCCTTATGCTGACCCTGAAGAGATCCGGCCCAAAGAGGTCTACCTGGACAGGAGCCTGCTGACCC
TGGAGGACAATGAACTGGGCTCCGGTAACTTCGGGACTGTGAAAAAGGGATACTACCAAATGAAAAAGTAAGCACCGTTGACTCATTGCACTGC
TGTCATCTTCAGTGTGAGAGTGCATTCTGAAAACTTCTCAGAACGTACCTACACTGCTCAATTAATCCCAGACATTGGCGTCCAAGTACGTGTG
TTTATTGCACGCACACACATACACATTGCAGCGCTTATGTGGTCAGAGGACTCATCAATTGGTTTTCTCCTCCTCACACAGGTTCTGGGGATTA
AGCTAATGTCATCAGGTTTGGCAACAAGTTTTTTTTTTTTTTTTTTTGTTTTTTTACCAACTGAGCCATCTTGTAGTCTTCATTTCTGACTCAT
TTAGAGTAAAAGCATCCTTTGTGTATGCTAACTGTGGTGACAGTAGTGACATGTTTGTATTAGAATGTAGTATAGTATAGAAAACAGCTAAATA
GAAATAAGCAAATTCATAAGAAAATAAAAATATATTCTATCTCCAGAACACAAATATCTGTGACTAACCTTGGTGTAGGCATTTATTGGATCTT
TATATGTTTTGTATGTAAATGGTAAGTGCTTTTGGTACTCTTAATAGTTTGCTGGTAATGTGTGTATCGTGTAGCATTTTCCATGTCTAGACCT
TATATATGAACTAATATTGATTTTACACAGAGATGTGACAACCGGCAACACAACTGAGTTCACTTGTGACCTGTAGCAGTTTATCTAGCTATCC
TTTAATACAGAAATGTGTCACTTAGGAATGCCAGATCTCTGTCTTTCTCTCTCTCTGTCTTTGACTCTGTCTCTGTCTCTCTCTCTCTGTATGC
ATACATGTGGTGGGTATGACTATATATGTTCAGATATGTTCACAAGTATGTGTGGGTTGTGAGTACACATTTGTGCAGGTACAAATGCCCTTAG
AAGTAATAATAATAAAATAATAATAATAGTAATAATAGTAATAATAATAATAGTTCCTCAGGCCCCGTCCACCTTGCTTTTTGAGGCAGGGTCT
ATCACTGGAATCTGGAGCTTACCAATTAGGCTATGGAAACTGGCCAGAGAAGCGCAGGAATTCACCTGTCTCTGCCTCCCCAGTGCTGCGATTA
TAGCTGTGTACCACAAACATCTTTTTATATGCTGGGGTCTTGGGGGTCGAAGCCCAGTCCTCATGATTTCACGGCGAGCCCTTTACAGACTGAG
CTGCCTCCCCAGCCCAATAGTGATACTTTTTAAAAGAAAACCATGTCTACTCCTCATCCGTTGCGTGTTTGTCCTCACCTTGGTGCATTAACTA
AGAATTGTAGAGTAGTGTTTTAGAACTGGCAGGAGTCTTGTGCCTGGTTACAGAGAGATCCCAGCATTCCTTTGGGTGTGGCTTATGTCTGTAT
GATGACTGCTGTCTGACAGACTGAAGACATGCTATTTTGTTCCGATAATTCTGCAAGTTCTTTAGTCCTGCATCTATGCTGNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNTTGTGCTTCTCTTTCTTTCCTGTGATGAGTTACAGCCATAGATTTCAGACCTTAAACATGTCCCTCCATCTT
GGGGTAAACTCCCTCACCTTTGTTCATTACATCTCCTTCCATAAGCAACTGAATATAACCCATGAGTATTTCTCCTAGAATTTTTCCTATCTGT
GCTCATGAAGTGTCCTAATTACTGTTCTATTGCTGTGATGAGACACCGTGACCAAGGTAACTTATAAAAGGAAGCAATTAATTGGGGGCTTGTT
TAGTTTTAGAGGGTGAGTCTCTGAACATCGTCACAGCAGGAAGATATGGCACTGGAGCAGTAGCTGAGTGTATATCTTATTTGCAAGTTGGAGG
CAGAGAGCAAGAAACCTCAAAGCTCACCCCCAGTTACAGATCTCTTCCAACAAGGACACACCTGCTAAACATTCTCAAAACAGTTCTACCAAAC
TAGGGGCCCAGCCTTCGGATATATGAGCCTACCGAGGTTTTTCTCATTAAACCACCACGTGAAGTTATCAGTCAATTGCTTTCTTACAATTCTC
ATAATGGAGTCTTACTCTCTTACCTGTAAAAGAGCTCAGTGCTTGGAAGACCAGGAGTTTCATAGTCTCTTCCTGGGCCACCGTAAACAGTCAG
GAATTGGAACAATAGTAGCCTCTTAGAATTAGTGGGATATTATCTTTCCCCTCTGCTTTTCTAGAAGACGTGGTATTTTCTTTTTTAAATGTCT
AGAGGAATCAAGAAACCTTTTCTAACTTCTTAATTTAGATCCCTGCCTCACACCACAGATGTAATGATAGACCCAACTATGAAAGCCAAAATAG
GTGGCTAGAGATGACAGAATGGAGCCTCCCATGTTCCCGGGAGGGCCCAGAGAGGCTTCTAGGGTGATGATAATATCTAAATCCTCCTGTCAGG
CCACAGGCTGTGTTCTAGATCACCACACTAGCTGTGTGTACTTTCCTGTGTGTGTGTGTCCCACTAATTCTGTTATTTGACCTAGTGTATTTGT
TACGTGTGTCTGTGTGTGTCTGTGTGTGTGTGTGTCTGTACGTACATGCATGTGGTTTGGGGATCAAGCTCCAGCCACCAAGCTTGGCAGAGAG
TATCTTTACCACCTGAGCCCCCACACGGCCCATTTCCTGAATAGTTCTGTTTTGATTCATCGGTTTGAAGGTTCAACAAAAGAAACAACTTCTC
ATGCTTGTGGAAATTTTACCACAGGTAATTTTTCAAAGCATACCTAGTTAATGTTTGTTCTTTCTTTTAAGCCACATTTTGCTAGAATACTTTC
AAGAGTTCAGCATGATCCATGTGATGTTTTTGTGAGGGAATAAAAAAAAATCCATATTTTCGAAACACATTGAGGCTTGCTTAAAAGTAACCAA
GCCCAGTGGGAGAGAAGTGTTTCTTTCTCTTTAACTCTGAGCTTGAACAGAATGTGTGCTTCTGATTGGGCATGTTGTGTACACACCTGTGGCC
CCCCAGAGCCCCAGAGGTAGAGACAGGGGGATCAGAAGTTTAAGGTCATCTCCTACTATACAGCTAGTTTCAAGCCACAGTGGATTATGTAAGA
CCCGGACTCAAAGAAAAAAAAAAAGCAAAGCAATGTATTTTTATTTTTTAAATTCTGGGCCCAGAGGCTCATTTGCAATATCATAAAGAAACTT
TGTATTCCTTTATGCATGTAGGCAGAACCTAGCTGGGAAACCACATGACACTGCAGCAGGCTGTATGTGTGTCACAACAGGGGAACTTTGTTGA
AGCTGGTTGAATATTTCACAATTCGGGGCTTTCCTCAGCCTCGACCATTCTCCATTGCTGTCCACTACACCGGGGTTCACAGGCAGTGGGGCAT
GGTTCCTCTGTCTGTAAAAGAACAGCTGTAGAAAGAATCTCTTGTGTACTGTATGGAATCATTACCATCATACAGAGCACCAACCACAAACCAG
AGAACAATCCCCGAAGTCCAATTTGGGGAACCAGTGACTGGGCACGCATGTGGGGGTTACTCACAGGAGCTGCGTGTGCCGCTGAGAAGTCTGG
GCCTCAGTGTGGATGGTGGCCTCTACAGGATGTAGGGTCCCCTAGATGCCCCCTCTCGGGCTCCTGTTAGCTTTCACAGTCGTATGCTCTAGCA
CCTCCCAAGACCGTGCGTCTATGTTCAGTTAGGACAGGATCTCACACAGATGACTGGGAAGCGCCGGGGGGAGTTCCAGGAATCAGGGTAACAG
TCCAGTGAGCATCTCCACCCCTTCCTTATATGGACGAGCACCAGCATTTGAAGCTGGTCTTTCTTCTCTCTTGCAAAAGGCATAGCTGATCAGA
TGGAAAGAGCAGGTGTTATATTCAGAGGACGTTAAAGATGATCTTAAACCAACGATCCTCCTGTATCCACCTCTGGGGCTCTGGGGCTCACAGG
TGAGCACCCCCATGGCCGGTCTGAAGCACACGAGTTGTTCAAGCTCAGAGTTAAAGGGAAAGAAACACTTCTCTCCCACTGGGCTTTGTTACTT
TTAAGCAAGCCTCAACATGTTTCGAAAATACAGATCTTTTTCAATTTCTCACAAAAATATCACATAGATCGTGTTGAACTCTTGAAAGTATTCT
AACAAACTGTGGTTTAAATGAAAGAATAAACATCAGCTATGTGTGATTTGAAAATTTACACCTGCCGGGCAGTTGTGGCACACGCCTTTAATCT
CAGCACTTGGGAGGCAGAGGCAGCCGGATTTCTGAGTTGGAAGCCAGCCTGGTCTGCAGAGTGAGTTCCAGGACAGCCAGGGCTACACAGAGAA
ACCCTGTCTCGAAAAAACAAAAACAAACAAACAAACAAACAAACAAATAAACCCCAAAAGAAAGAAAGAAAGAAAGAAAGAAAGAAAGAAAGAA
AGAAGGAAAATTTGCACCTGTGATAAAATTTTCACAAGAAGGAGAAAAATGGTACTAGCTATTTATCTTGATGTGTGTGTGTGTGTGTGTGTGT
GTGTGTGTGTGTGTGTGTGCAGGTACTCCTGACACAGCACATATATAGAGGCCAAGGGACAACCCTTGGATGTCACCTCTTACCTTTCACCTTG
TTTCAAACCGCCTCTGGGGATTCACTGCTGGCCCCACGAATCTCCATAGATTCTCCCCTAGAAGCCCTGGGGTTGCAGACATCTGCTATCCAAC
TTTCTGTGGGTTCTGGGGATCTGAACTCTTAACCTCACTCTTGCATGCGCTTTTTACACTGGGTTATCTCCCCAGCCCATGGCCGTTTCTTGTT
TGTTTTTTTTTTTTCGTTTGTTTGTTTGTTTGTTTTTGGGTTGCTTTGTTTTGTTTTTCTGAATGGCTTTAAGAGATTCTCATTGGGCAGCGGG
TGTCAGCACCATTCCCCTCCACCCCCCTCCCCCCAACACACACACAATGTGACTAGAGAGAAGAGGACCTCTAGGCAGTTTGGAAAGTTTCTCA
CAAGTTACAATCTGTGTCACAGAGTTGTGAAAACCGTGGCTGTGAAAATCCTGAAGAACGAGGCCAACGACCCGGCTTTGAAGGACGAGCTGCT
GGCAGAGGCGAACGTCATGCAGCAGCTGGACAACCCCTACATTGTGCGCATGATCGGAATCTGCGAGGCGGAGTCCTGGATGCTGGTGATGGAG
ATGGCGGAGCTGGCGCCGCTCAACAAGTACCTGCAGCAGAACAGGTGACCTGGGGCATGCGAGTTGTGTGCGGTGCCACCCCGCACATGCCCTG
TCCCCCGAGTTTGGCCTGTGCCGGGGCCCCGCCCCCACACACGCCCTGTCCCCCGAGTTTGGGCTGTGCCGGGCCTGATTTCTAGGGGAAGCTC
TCTCTCCCTCTCCTCTCTGTGTCTCTGAGTCTCTGTCTCTCTGTCTCTCTTCGTGTCTCTCTCTCTCTCTCTCTCTCTCTCTCTGTGTGTGTGT
GTGTGTGTGTGTGTGTACATCTTTGAGGGTGTGCACCTGTGTGGATGTGTTTATGTGGAAGCCAGAGGTCAATATTGGAGATCACCCTCTCTCC
TCTCTCAGTCTCCAACTTAATTTTTTTTTTTTATTTATGTGAATGTTGGTGTGAGTGTGTGCCACACAACGCAGATACTTATGGAGGCTAGAGG
AGCTGGTCAGATCCCTCGGAGCTGGGCTTACAGCACTTGTGAGCCTCCCAACATAAGTGCTGGGAACCAAACTCCGGTCCTCTGCAAGAACAGT
ACGTGTAGCCAGGCAGTGGTGACACAGGCCTTTAATCCTAGCACTTAGGAGGCAGAGGCAGGTGGATTTCTGACTTTGAGGCCAGCCTGGTCTA
CAAAGTGAGTTCCAGGACAGCCAGGACTACACAGAGAAACCCTGTCTCGAAAAAAAACCAAAAAGGAGAAAAAAAAAAGAAAGAAAAAAGAAGA
AAAGAAAAGAAAAAAGAACAGAAGGCGTTCTTAACCACTGAACTATCTCGTTAGCTCTCCGCCTTATTTCATGAGACAAGGAACTATCTCATTA
GCTCTCCGCCTTATTTCGTGAGACAAGGAACTATCTTGTTAGCTCTCTGCCTTATTTCGTGAGACAAGGTCTCTCACTAACACCGGAGCTCACC
AATTCAGCCAAACAGAGCCCCAGGGATTACAGGTTCTCACTGGCATACCTGGCTTTTATATGAGCACTGTGGGTCCCAACTCAGGCTTCATCCC
TATAAGTCAAACAGTTTACCAACTAAGGCACCTTCCTAGCCCTGTCCAGTGATAAAATCCTCAATGTGTGTGGACAGGGCATTGCCCACCAGCA
GGCAGAACATTCTTCTTTGGTAAACACCTGGAACAAACATGTTCCTGGGGCACCATCTGTGTCTCGTCTGGCCGACCTCTTGAGTGCCTGCATC
TGAAGAGGCAGCAGCAGTTCGTTGATTCTGGTCCAGACATAGGTGCTCTAATGCTGCGGTCAACATAGAGTAACTCCCCGATTCCTCTGAGCCG
GGGTTCCTTCTGTACAGCTTTTCCTCATCATTTCCAACATGGATGTGTGAGTTTGAGCATGTGCAGCAGGTGGAGGCCAAAGGTCAAGCCAAAC
CCATGCCCATCCCTTAGAATACCATCTGCTTCCTCTGAGACAGAGGTCCCTCATGGGCCAGAGCTCATCAGTTTAGCTAGACTTTCTGGGCAGG
GAACACCAAGAATCCACCCGTTTCACTTACCCGGCTTTGGGATTACAAGTGCAGGCCGCCCGCCATGTTTGGCTTTTATTTCACGAGGGCGCCA
GGCATCAAACTCAAGTCCTCTTGCTTGCAGGATGAGCGCTTTCCTAAAAAGCTTTCTCCATAGCGCTATGAATTTTTGTCCATCTCAAAGCATG
CCTGAAAATGAAGCACACTCATCATATAGTGTGTGTATGTGTATATGTGTATGCACATGTGTGTATGTGTATATGTGTATGCACATGTACATGG
GTATGCATGCCCATGCATACGTGCAGAGCCCAGGACAGGCCACTGTGTGTGTCCTTCTCAATCACTCTCTGCTTTATTCCCTCGAAACACAGCC
AGGTGTGGTAGCACACACCCTTAATCTCAACACCAAAGAGGCCGAGGTTGGTGGCTCTCTGTGAGTTTGAGGCCAGTCTGATCGACACAGTTTA
TTCCAGGCTAGTTGGGGCTACAGAGGGAGACCTTGTCAAAAACAAACAAACAAACAAACAAAAAAGGTTCTCCCACAAACAGAAAGTCCTGCCT
GTTTGGTTTGGCGTGGTTTGATTTCGTGTGCTTTTGGTTATTCTCATAGCCAGCAAGCCTCCGCCTCTTCCTGTCCCCATCCCCTCCCAGTGCT
GAGATTACAGCACAGGTGACCACACTCAACTTTCTCCCATCACTTCTGGGGATCGAAACTTAGGTCCTCAGGTTGTGCACGCAGCAACTGTCCT
CGCCCTGAGTCATCTTCCCAGCCTGTCCTGTTGTCGACAAAGAAGGAACGAGTGGACCGACCCTTCTTCCATGTACTCGGGGGACACGGCTGTG
TGTTTGGAATGGCCGCCGTCCACTCTTGGTAATGATGTCGTGTGATTCTAGGCACATTAAGGATAAGAACATCATAGAGCTGGTTCACCAGGTT
TCCATGGGGATGAAGTATTTGGAAGAGAGCAACTTTGTGCACAGAGATCTGGCTGCGCGGAACGTGCTTCTGGTCACACAGCACTATGCCAAGA
TCAGCGATTTCGGTCTTTCCAAAGCCCTGCGTGCTGATGAAAACTACTACAAGGTAGGAGCGCTCGGTGCAAGCTCACAGAACAGGGAGAGGAA
ACAGCTCTGTGCAGCCTCACGGAGCAGGCTGAGAGCTCAGTACAGGCTCACAGAGCAGCTGAGGGGACAGCTTCCATGCATAGTTAAGGTTGCA
GGTTATAAAGATATAGAAAGATAAATGTGTGCTCTCTGTATTTTACTATGGTAGAAATCTCACTAGTAACCTTGGAGAAAAGTATTCTTAAGAT
CGATTCTGTCCATCAAGAAACACAAACCAAAAATACTGTAATGTTTCTCAACTGGAATCTTTTTCAGACTAGGATCTTATGTTGAGTTTTTTGG
TGGTGGTGGTGGTTTTTTTTTTTTTTTTTTTTCGTTTTACATTTACTTATCTTGCTCTGTGTATGGAGTCATGTGTGCCGTAGTGTTCTAATAG
AGAGCAGAGGACAACTTACAGAAGTTGATTCTCTCCATCTACCATGCATATCGGAGGAATCGAACTCTGATCTTCGGATTCGGCAGCAGGCACC
TTACCCATTGAGCCATCTTGCTGGCCTCTAAATGCTTGCGTCAACTTGCTTTGAAACATACAAATCAAACAAATTGATTTTTCTTTTCTTAGGC
AGGGTTTCATGGAGCCCAGGTTGGCATCTGTCTCCTGAGTGCTGGGGTTATAAGTATTCACCACCACGATTGGGTTACTCAGTCTACCTAATAC
TATGACCCTTTAACACAGTTCCTCGTGTTGTGGTGACCCCCAACCTAAAATCTTTCATCGCTACTTCATAACTGTAATTTCGCGACTGATATGC
AGGGTGTCTGATATGTGACCCCCAAAAGGGTCACTACATAGGCTGAGAACTACTGGGGGCTTTATGCGTGTTAAAGGAAACCACTGCTAACTGA
GCCACTTCCTTGGCCCCAAAACTGGTGGATCCTTGAGAGATTTTTCCGAGTCCTTTTCACTGAATTGACACTGTGACTTTCAGCAGAGGCTTTT
TCCTACCCCACTCCTTAAACCCAGCTCCTTTGTGCATCGGGGTGTGCACACCTGACTTAACGCATCACAGAGTGTCCACTCTGCTTCCAGGCCC
AGACCCACGGGAAGTGGCCCGTGAAGTGGTACGCCCCCGAATGCATCAACTACTACAAGTTCTCCAGTAAGAGTGACGTCTGGAGCTTCGGAGT
CCTGATGTGGGAAGCGTTCTCCTATGGGCAGAAGCCCTACAGAGTGAGCCATGCWTAGCTGTGTGTTTTGTGTCTCAGCCTCTTGGACTTAAGA
AACCATCCCTCAGTATGGGGTTATTGCCACAAGAACCTGAACCCCTGGAATCTGAGGACACCCTGGGGTCTCCCATCACCTTCTAACAGTGGGC
GGCTAGTGCAACCTAACTTACGCTTGTACTCACTTGAGAATGACATGTGATACTGAGTGAATGCATTCTCACTGTGCTACACACATACGTGCAC
CAGGCACAGGCCATCCTTCCTGAGAGTTAAGTGGAAATGGTGATACCGATGGATGACATCAAGTATTTTAGTTGTAAGCGTGTGTGTTCTCATG
TGTGTGCACACCTGTGTATGTGCAAACACATTGTGTGTGTGTGTGTCTGTGTGTGTGTGTGTGTGTGTGTGTCTAGCCCAGAGACCATCCTCAG
GTGTCATTCCTCAGGGACTCTCCACTTTGCTTTTTGAGACAGTGTTTTCATTAGCCTAAGCTCACCCAATAGGCCAGGCTGGCTAGCCTGTGAG
CCCCAGGGATCTGCCTGTCTCTCTACCCAGCACTGGGCACACAAACATTCAATACCACGNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNCCAATCATGGTGTTTCATGGCAGGCATACTACTATATGCATTTTGTAACTGTGGGGATGTCATAAAGCATACGTAAGCA
AACTAGGGCAGCTGCAAAGTCGCCAGCTGTTACAATCTTATGAGACTGTGATGTGGATTGGGAGAAGTCACAGAATCCCAGATGTCAACTCAAG
CATCTAGAACACTCTTGCCTTCCACAAACTCAGACATGTCTGCGAGCATCACTGCTTCCAAGAATCCTAACATCGAGGCTAGATGCTCCTGCTG
ACCACAGACAGCTCAGTACCAGAAGTCTCCCGATAGTGGTGCAGAGTTGCCACTTGCCACGTTCACCACTGCTCAGCCCATAGGGTCGGAAGCC
CCATCCCTATTCTTGGGTACAGTGTATTTTGCCTTCCCCTCCATCCCCTGAGCAGGACAGACACCGTGCACGTGGTGAGAGCGTGAATCCACTG
TGAGCCACTGGGCATGCTCTGCCCTACCTATGGGAAGGATCCCAGCCACCGTGATAAAGCTGTTCGGTGCTAGTGAGGCACTCGATGTTAGAAA
GCTGACATTGAATGTAGCCATACATTTTTTTTGCAACGTATTTTTAATAAAGGTTCAACCTCCCCTCTAGCCCGCCACTCAGCAGAGGTAGCAG
AAGATAAAAGTTATTCGGCTATGGGGGAAGTGGACCTGTTCAGCAATAGTTCTTTGGGGGGCGAGCTTGATCCTCTTGGGCAGCAGCTCAGCCC
TGTAGCAAACACCAAATGTGATTCAGCAGCTGCAGACCAGTCCGCTAGGCAGGCAGACACCAGGCATGAACCAGCAGCTACAGTTAAGTCTTTT
CATCGCTTGCTTTCATCGCTTGCTTGCCTCAGCATCTAACCTGTAGGCGACAGCACAGACTTGCTGGGCAAAGACTTAGGATGCACAGGGTTGG
GCTCTGAACCAGACCACAGCCACAGAAATCTGTGAGTTCTAGCAAGAGCACAAACCAATACCCCCTCTCCTTGAAAGTCCAGCTTGAACGTGGG
TATGGTATATTACAGCTGTGTCCATATTCTCCTCTTAAGATTTTTAGCCAAGGGACTAAGGAGATGGCTCTGTTGGTAAAGAGTTACTGTTGTG
TAACTCTAGGGACCTCACTTCAGTCCCTAGTACCCATATAAAATACACCAGGCCTGGTGGCAAGTGCTTGTGGTCCCAGCACCAGCAAGGAAGA
TGGAGACAGGAAAATCTCAGATGCTTTCTGGTCAGCCAGCCTTGCCAACTTGACAAGTTCTAGGCAATTGAAAGACCCTGTCTCCAAAAAAAAA
AAAAAAAAAAAAAACCAAGGTAGATGGTGTCTGAGGAAGGACACCAAGGTTGTCCTCTGGCCTTCAGACACAGAAGATATTCTGTACCATAGTG
CAAAACTCTTCTGGCTCACAAGGACATGGATCCCCAGGATGCTATAAAATGCAGGCTTGAGTTCTGTAAGTTTAGAGCAGGAAGAGACATTTCT
GACCATGCTGGAGGCAGGCGCAGTGAGCAGCATTCACCACTGCCAGGAGGCACGGCGACTTAATCATGTTTCCATGAGCCTATGGATAAACACT
AATACAGCTCTCTCTTCCTTCTTCAGTGTGGAGAACAGGCCAGGATTCACAGCTGTGGAACTGAGGCTTCGCAATTACTACTACGACGTGGTTA
ACTAACAGCTCCGGCGCCTGTCCGTGCACACCACGGATTCCCAAGCGATCACAGGAAATTCATTCAGATGAACTGGCTCTCAGAGTTTCATCTC
CCTCTGCCCGGAGTGAGAGCTAAGCTAACATAGGACTCACCCTCACAACAGGTCTGGTGCCCAGAGACAGACAAGCAGCAAGACCTTGGGGCCT
GTGGAGTCGTCTCTCTGGTCTTTGTTTCCATCTGTGTGGTCTTCGCTATCGGTCACGTTTGGGAGCCATTTCCAAATTCTCTTGACATCATTCC
GTTCCTCTGGGTCCAGGATTTCAGTGTCCCCTGAAGATCAGAAAAGAAAAGTGCTTTGCCACTCCAGCAAAGGACATAAAGAGAAACCTCGCTG
GTGCAGTCAGCAAGCTGGAAGGTGGTTAAGGACAATGACAAGCCTGGACACTGGTTTTACTCTCAGCCCTGACACACAGATACTGTGTGGATGA
CATGCTGAGATGTGCTGGCTTGGCTCACACGCCCCTGGAGTCCACCTCCCTGCAGTCGTGTGTGTTTGCTGGAGGAGGTGTGAATAACGTTCTG
TGTGGAGGCTGCCGGGGGAGGTGAGCTCCCATGATCCTCTGCGACTGAGCAGAAGTGTGTCAGGCAAGGGTCAGACTCCCCTCTCTGCAGAGGG
AATGCATCCAGGACTCCTTGTTGCTGCCACTAAAAAATTGCCAACCAGCAACTTAAGGGAGGAATGGCTCATTTGAGCTCACAGTTGGAGGGGA
TAGGCCATCATGGTGGAAAAGACAACAGGCAGGAAAGGCTTGGTGGCATGAAAAGGAAGCTGGAAGGTCACATGGCATCCACATTCAGGAAACA
AAGAATGAACAGGAAGTGGGGCCAGGCTATAAGGCCCTAAAGGCCACCTCCGGTGACCCAGTTCCTTCAGATCCATCTATCAAAGATTCCACAG
GCCTCTCAGACACTGAATGCCAACAGCTGGGGACCAAATGTTCAAGCACACAAGGTGCAGTGCCAGGCAGGGGCCTTCCTGAATGATTACACAG
TGTGGTATCCTGACAGAGCGGGTGAGCCATCTCAGGTCTGTCCCCTTATGAGCCCCGTTTACCATCCCAGATTCCCCCTTGGGACCCCCCTCAT
CTGGATCATTCCCCTCCCCCCAGGCCCTATCTCCAAACACCTAGGTACTAAGTGAACCTAGGTACTAAGTCTTCAACACATGAATTCTGAGGGG
ACACATTCATACTGGAGCCGATGGCAGCCTGGTGACCAAGACCATCTCAGGATGAGTTTCTGAATGGAATCTTCATTCGCCATAAAACCAGAGA
TGTGACCTTCCCCACAAGGAAGCCTTGCTAAGTGCGACATTGAGCTGAGGACACACAAGCTGAGAATTCCCCCTCTGTCCTCCTAGCCTTGGCT
AACTACTGACCATACACCGAATGTCACCCATTCACGGCACTTTTGCTGAGGACCTAAAAGCTACAAAGGCTGCATAGCCTTCCATCTAGGTCTA
GGGACATGTACAGATGGGAGCATCTCACCTTCACCTCTGAGACACTCGGACATTCACAGCCAGGGATGGTACAGGAGTCCCTGCTGGTGCTGGG
CTCAATGAACAGAGACGATGGTGAAATGGGTGCCTCTCTTTAGACCAGTGGTTCTCAACCTTCCTAATGCTGAGATCTTTCAATACAGGTCCTT
GCGTTGTGGCGACTCCCAACCATGAAGTTATTTTGTTGCTACATTGTCACTGTAATTTTGCTACTATGATGAATCGTAGTGTAGATATCTGATA
TGCAGGATATCGGATATGTAAATATCTGGTATGCAGGATATGTGAAATGAGGGCACATCCCACAGGTTGGGAACCACTGCCTTTGATAGGGAGG
CACTATGCTTAGAGCATGGTTAGGAAGTATTTAGAAGCTATTGGTGTGTCTTGGAATTGTGTTAGTAGGTCAACCGTCCTGTGGAGGGAAGGAA
GGGGGTGGAGGAAATCCCTGTATGTTGAATTCATTGACCAGACCTCCAGAACACAGCCTTGGTGAGGAAGTCCCATGGGTCAGACCACCTGTAG
ATTTAGTCAGGAGACGAAGGGCCACTGTATAGTTATGGAGACAGGACTGTCTGCTCTGAGCAAGAAGTTCTCTGCTGAGCTCCTAGCCTACCTT
CCCTTGTCCCCAGGGCTATAGAAAGGCCACCTCGAAGACCAGGGAGCATGTGTGAGAAGTCTGTGGAGGCCTCTGCCTTCTTCCTGGCCTCTCT
AGCAGTGCCTCTCCTTCCCAGGGATTTGGGACTGACCGTTATCTGGCTATGACTGAGCAGAGTGGAGGGATGGGCTTTGCCCCCCTTCAGAAAG
CCGAAACCGGTTCCCATGTGCAGAGTCACATGTGGCCATCGACCAGGGCTCTCATCTGGTGAAAACTGTGTCTTACTGTCAAGATTAATTCTCT
CCCCTGGGCCACTGAGGAGGGCAGATGGCAAGAGGATCCTCCCTAGAGGATTTAGACTATGAATGCCCACTAAATTTGCAAGGTCAGAAACTAG
CCAAGGTCCTTCTCAGGCATCTATCCTTAACTTGGTCTCTCCACAAGCTGCCTCTGATGCTCCGAGCCCCTTCTGCCTGGCAGCTGGGCTCCCT
GTGTGCTTTTACCTATGACCCTTGGTCAGCAGAGTACAAGGAGCACCTAAGCTGCCTCGCCACCTCCCACTGACTGCAAACACCACATACCTCA
TGCGGAACCGAATCTCTCTCCAGTAAACACCCCAGCCAGCCATTCATAAAAACCTGTCTCTGTGTGCGCTGGGACATGTCCTCCCTGTACCCCG
GGCCTGCTTTGTGTGCCAGACAGTGACTCCACAGGGATGCCAGGTCTGTGATTGCATTGCTCCTTGCAGAGGGAAGCCCTCGGTTGTCCTTCTC
TGGGAATGCTGCCACCCAGAGCAGCACACAGTGATTCCCTGTTATTTCAGAAATTATTTTAAATAAAGATCTACTATTAGTCTTGTGATGACCT
TTACGGATGCAGACAGGAGATGATGGGATATACACCCTTGACTAGACCTGGCCACCATCTTCGTAAGTGCAACCTGCTTGTGAAATACTGAGCC
CTCAGTCATACCACTCGCTACATTTGGGTGGGAACGCAGACTCCAGAGCTCACCTATTCTGCCGCTTCAGAAGCCTGCAAGGAGAGCCCGGTCC
TGTGAGGGGAAACAACTGTGTCTGAGATTCGTAGGCTGAGAACTGACAGCCGAACTCAAACCCAGTAGAAAGTGGTACAAGGAGACCCCAAGGT
GGCCAGAGAGGGCGGGGTGAGCTTTGCCACTGTCAATGAAGACAACACGAAAGCATCAGAGCAGAGAAGGTCAGGATGTAAAGGTCCCGAGCCC
TGGGCTCCTCTCATTCTATAGGCAGTTATGAGCAGGCACACATACATACACTACATATAATTGCATGTGTGCATAGACAAGCATCTTATGTATG
TATACACAGGAACACATACACATGCATGCACAAACGTGCATTAAAAACATGCCTGCAAATGCACACTGCACACACAGACATGAACCTGTGTACA
CAAATCCACAATCACATGTACATGTTGACACATCCCCACTCGCAATGCCACTTTGCCCACAGGCTAATCTGATCTTGGAAATCCCTCCATGGAG
AGTCCCTTCCCAAGTGACTCTAGGCTGTGTCAAGTTCATAGTTTAATCTAAGCATGGTACAGAGAGTGTATGGTAAAGAGAACCTGAGTTTAGC
CCCCAGTGCCTATGTAGAGAGGCAGTGCATTGGCACATGCCCGCAGCGCCAGCTCTGGGGAGGGAGAAGCAGATCGCTACAGCTCACTGGCCAT
CCAGCCTCACCCAGCACGGTTGTCACATTCCAGGTCTTATGATCCTGTCTTAGAAAAATAAAGGAGAAAGCAGCTGAAGAGAACACTCAGTGTC
AACCTTCTGCATGCACACATACAAACATGCACCCCTGAACACATGCACCCCTGAACACATGCATGCACACACATGCATACATACACATGCACTG
GCACACATACAAACATGCACCCCTGAACACATGCACACATGAACACATGCATGCACACACATGCATGCACACACATGCATACATACACATGCAC
TGGCACACATACAAACATGTACCCCTGAACACATGCACACATGAACACATGCATGCACACACATGCATACATACACATGCATTGGCACACATAC
AAACATGTAGCCCCGAACACATGCACACACACAAACATGCACACATACAAACATGCACACATGAACACATGCATGCACACATGTGCACACAAAC
ATGCACATGTGAACACATGCACATGCATGCACACACTCATGCACACACACACATACACATGAGCACACACATGCACGCACGCATGGCAAAGAAC
GCAATGCCTCTGAACAACCTCAATTTAAGAGCATTGCCTGCACACGCATCAACACTTGTCTATCCATACATGTGGACTTGTGTAAGAGCCACAG
ACTTTTAAAGCTGGCAGGCTCCCACACTGTACCAATTGGTTTCCCGGTGCTGTGACAAATACCTGAGTAAAGCAACTTAAAAGGAGGAAAGGCC
TGTCTTGGCAGCAGTCTCTGAGCCCTTGTTCCTGGAGTGAGGGTTCPAGCCCAGGCTCTTGGGCAGCCTACCAAGCCACATCCAGCCAATGCAG
GATACAGAGGTGCAAAGCAGAGACTGGAGGTTTAGCCAATGTAACCACATCAGAGATAGGGACAAAGATGGTTGCTGACCCTCCTAAGCCCATC
TTCTGGGGTCCTGGCACGAAGCTTAGGTTTAAAGAGAGGACCAGCAACATGTCCCCATGGAGGCATGCTCCCATCTGGCAACACGTTTCAGCTC
CAGTCTCCCATGAGCAGCACCCTGACACAACCTCTCCCCAGGAGACCAGCTCCCCTCCTGGCTGGCACCGGGCTTCAGCCTTATCCTTCTCCAC
CAGCTCCTTGGTTTGCAGCTTTGCTGCTCTGATGGTCAAAGGCAGATGCGTGGGGCTCTCGCTACAGCAACGTTATTCCTGCCAGGATGGTAAA
GCTGTGGGTACCTGACGCTGCTGATTTGTGTCTGTGGTGGCATAGCTTCTCACCAGAGGAGCACACCGTGGAGGAAGCTCCTCTCTTACTGTGG
TCAGGAAGCAGAGAGAGAGGGAGAGCGGTTCCCAACATCTACAACAGCATTCCTGACGAACGGCTTCCCTTTCACAAGGCCCCAGATCTTTGAG
TTTATTATTTTATTTGTTTGTGTGTATGTGCATGTTCACACACATGTGCACCCGCGCATGTGTGTGTGTGCTTGTGTATGTGTGTGCTATGTGT
GCAAGGGTTCCTTCTGAGGACAGAAGAGANNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNN
MOUSE SEQUENCE—mRNA
GGCACGAGGGAGGAGAAGGCGGCGGCGGAGAAACAGGAGGAGGGAGGAGGAGAGGGGGTTGGAGGACGCGGCCACCTGCCCAGGTAGGAGCCAC
TCCGCCTCGCCGCACCCTAGCAAAGGGCCTCGGCACCCCCAGGCGACAGACCTTGGCTAAACTAGGCTGGACCGCAGTTTTGCTCCAGTGACTG
CGGCTGAGCGCGGACCCGCCGGGCTCCGGCGCGGGACCAGAACTCCCGCCTGCAAACGCGCGCGCCCGGGCGCGGAGTGGGGCCCAGCCGGAGC
TCCGCCTCTCGGCAGCTGGAGGATCGGAGAACCGGGAGACTTCCACGGTGCGCGCCCCCGAAGCAAAACAACGTCCCCAAGCATTTGAGATCCG
GGACTGTCGTCGTGCGCGCCTGTGAGTTGCTGCAGAGTCCCAGCGCCTCTGAAATGCGGTACTTCTCCATACACTTCCCAGAACTCTGAAGGGG
TGCAGACATGGCGGGAAGTGCTGTGGACAGCGCCAACCACCTGACCTACTTTTTTGGCAACATCACCCGGGAAGAGGCTGAAGACTACCTGGTC
CAGGGAGGCATGACCGATGGGCTCTACCTGCTACGCCAGAGCCGCAATTACCTGGGTGGTTTTGCTTTGTCGGTGGCTCACAACAGGAAGGCAC
ACCACTACACCATCGAGAGGGAACTTAATGGCACCTACGCCATCTCCGGGGGCAGGGCCCATGCCAGCCCAGCAGACCTCTGCCATTACCACTC
CCAGGAACCTGATGGCCTTATCTGCCTCCTTAAGAAGCCCTTCAACCGGCCCCCGGGAGTACAGCCCAAGACCGGACCCTTTGAGGACCTGAAG
GAGAACCTCATCAGGGAATATGTGAAACAGACCTGGAACCTTCAGGGCCAGGCTCTGGAGCAAGCCATCATCAGCCAGAAGCCCCAGCTGGAGA
AGCTGATCGCCACCACGGCCCATGAGAAGATGCCCTGGTTCCATGGCAACATCTCCAGAGATGAATCAGAGCAGACGGTCCTCATAGGGTCAAA
GACCAATGGAAAATTCCTGATCAGGGCCAGAGACAACAGCGGCTCCTATGCTCTGTGCCTGCTGCACGAAGGGAAAGTATTGCACTACCGCATT
GACAGGGACAAGACCGGGAAGCTCTCCATTCCTGAGGGGAAGAAGTTTGACACCCTCTGGCAGCTAGTGGAACATTACTCTTACAAGCCAGATG
GGCTACTAAGAGTCCTCACGGTACCATGCCAAAAGATTGGTGCACAGATGGGCCACCCAGGAAGCCCAAATGCCCATCCCGTGACTTGGTCACC
GGGTGGAATAATCTCAAGGATCAAATCCTACTCCTTCCCAAAGCCTGGCCACAAAAAGCCTGCCCCACCCCAAGGGAGCCGTCCAGAGAGCACT
GTGTCCTTCAACCCCTATGAGCCAACGGGAGGGCCCTGGGGAGACAGAGGCCTTCAGAGAGAAGCCCTGCCCATGGACACAGAGGTGTACGAGA
GCCCTTATGCTGACCCTGAAGAGATCCGGCCCAAAGAGGTCTACCTGAACAGGAGCCTGCTGACCCTGGAGGACAATGAACTGGGCTCCGGTAA
CTTCGGGACTGTGAAAAAGGGATACTACCAAATGAAAAAAGTTGTGAAAACCGTGGCTGTGAAAATCCTGAAGAACGAGGCCAACGACCCGGCT
TTGAAGGACGAGCTGCTGGCAGAGGCGAACGTCATGCAGCAGCTGGACAACCCCTACATTGTGCGCATGATCGGAATCTGCGAGGCGGAGTCCT
GGATGCTGGTGATGGAGATGGCGGACGTGGGGCCGCTCAACAAGTACCTGCAGCAGAACAGGCACATTAAGGATAAGAACATCATAGAGCTGGT
TCACCAGGTTTCCATGGGGATGAAGTATTTGGAAGAGAGCAACTTTGTGCACAGAGATCTGGCTGCGCGGAACGTGCTTCTGGTCACACAGCAC
TATGCCAAGATCAGCGATTTCGGTCTTTCCAAAGCCCTGCGTGCTGATGAAAACTACTACAAGGCCCAGACCCACGGGAAGTGGCCCGTGAAGT
GGTACGCCCCCGAATGCATCAACTACTACAAGTTCTCCAGTAAGAGTGACGTCTGGAGCTTCGGAGTCCTGATGTGGGAAGCGTTCTCCTATGG
GCAGAAGCCCTACAGAGGGATGAAAGGGAGCGAAGTGACCGCCATGCTGGAGAAAGGAGAGCGGATGGGGTGCCCTGCAGGATGCCCGAGAGAG
ATGTACGACCTGATGAACCTGTGCTGGACTTACGATGTGGAGAACAGGCCAGGATTCACAGCTGTGGAACTGAGGCTTCGCAATTACTACTACG
ACGTGGTTAACTAACAGCTCCGGCGCCTGTCCGTGCACACCACGGATTCCCAAGCGATCACAGGAAATTCATTCAGATGAACTGGCTCTCAGAG
TTTCATCTCCCTCTGCCCGGAGTGAGAGCTAAGCTAACATAGGACTCACCCTCACAACAGGTCTGGTGCCCAGAGACAGACAAGCAGCAAGACC
TTGGGGCCTGTGGAGTCGTCTCTCTGGTCTTTGTTTCCATCTGTGTGGTCTTCGCTATCGGTCACGTTTGGGAGCCATTTCCAAATTCTCTTGA
CATCATTCCGTTCCTCTGGGTCCAGGATTTCAGTGTCCCCTGAAGATCAGAAAAGAAAAGTGCTTTGCCACTCCAGAAAGACATAAAGAGAAAC
CTCGCTGGTGCAGTCAGCAAGCTGGAAGGTGGTTAAGGACAATGACAAGCCTGGACACTGGTTTTACTCTCAGCGCTGAGACAGAGATACTGTG
TGGATGACATGCTCAGATGTGCTCGCTTGGCTGACACGCCCCTGGAGTCCACCTCCCTGCAGTCGTGTGTGTTTGCTGGAGGAGGTGTGAATAA
CGTTCTGTGTGGAGGCTGCCGGGGGAGGTGAGCTCCCATGATCCTCTGCGACTGAGCAGAAGTGTGTCAGGCAAGGGTCAGACTCCCCTCTCTG
CAGAGGGAATCCATCCAGGACTCCTTGTTGCTGCCACTAAAAAATTGCCAACCAGCAACTTAAGGGAGGAATGGCTCATTTGAGCTCACAGTTG
GAGGGGATAGGCCATCATGGTGGAAAAGACAACAGGCAGGAAAGGCTTGGTGGCATGAAAAGGAAGCTGGAAGGTCACATGGCATCCACATTCA
GGAAACAAAGAATGAACAGCAAGTGGGGCCAGGCTATAAGGCCCTAAAGGCCACCTCCGGTGACCCAGTTCCTTCAGATCCATCTATCAAAGAT
TCCACAGGCCTCTCAGACACTGAATGCCAACAGCTGGGGACCAAATGTTCAAGCACACAAGGTGCAGTGCCAGGCAGGGGCCTTCCTGAATGAT
TACACAGTGTGGTATCCTGACAGAGCGGGTGAGCCATCTCAGGTCTGTCCCCTTATGAGCCCCGTTTACCATCCCAGATTCCCCCTTGGGACCC
CCCTCATCTGGATCATTCCCCTCCCCCCAGGCCCTATCTCCAAACACCTAGGTACTAAGTGAACCTAGGTACTAAGTCTTCAACACATGAATTC
TGAGGGGACACATTCATACTGGAGCCGATGGCAGCCTGGTGACCAAGACCATCTCAGGATGAGTTTCTGAATGGAATCTTCATTCGCCATAAAA
CCAGAGATGTGACCTTCCCCACAAGGAAGCCTTGCTAAGTGCGACATTGAGCTGAGGACACACAAGCTGAGAATTCCCCCTCTGTCCTCCTAGC
CTTGGCTAACTACTGACCATACACCGAATGTCACCCATTCACCCCACTTTTGCTGAGGACCTAAAAGCTACAAAGGCTGCATAGCCTTCCATCT
AGGTCTAGGGACATGTACAGATGGGAGCATCTCACCTTCACCTCTGAGACACTGGGACATTCACAGCCAGGGATGGTACAGGAGTCCCTGCTGG
TGGTGGGCTCAATGAACAGAGACGATGGTGAAATGGGTGCCTCTCTTTAGACCAGTGGTTCTCAACCTTCCTAATGCTGAGATCTTTCAATACA
GGTCCTTGCGTTGTGGCGACTCCCAACCATGAAGTTATTTTGTTGCAGATTGTCACTGAATTTTGCTACTATGATGAATCGTAGTGTAGATATA
TGATATGCAGGATATCGGATATGTAAATATCTGGTATGCAGGATATGTGAAATGAGGGCACATCCCACAGGTTGGGAACCACTGCCTTTGATAG
GGAGGCACTATGCTTAGAGCATGGTTAGGAAGTATTTAGAAGCTATTGGTGTGTCTTGGAATTGTGTTAGTAGGTCAACCGTCCTGTGGAGGGA
AGGAAGGGGGTGGAGGAAATCCCTGTATGTTGAATTCATTGAGCAGACCTGCAGAACACAGCCTTGGTGAGGAAGTCCCATGGGTCAGACCACC
TGTAGATTTAGTCAGGAGAGGAAGGGCCACTGTATAGTTATGGAGACAGGACTGTCTGCTCTGAGCAAGAAGTTCTCTGCTGAGCTCCTAGCCT
ACCTTCCCTTGTCCCCAGGGCTATAGAAAGGCCACCTCGAAGACCAGGGAGCATGTGTGAGAAGTCTGTGGAGGCCTCTGCCTTCTTCCTGGCC
TCTCTAGCAGTGCCTCTCCTTCCCAGGGATTTGGGACTGACCGTTATCTGGCTATGACTGAGCAGAGTGGAGCGATGGGCTTTGCCCCTTCAGA
AAGCCGAAACCGGTTCCCATGTGCAGAGTCACATGTGGCCATCGACCAGGGCTCTCATCTGGTGAAAACTGTGTCTTACTGTCAAGATTAATTC
TCTCCCCTGGGCCACTGAGGAGGGCACATGGCAAGAGGATCCTCCCTAGAGGATTTAGACTATGAATGCCCACTAAATTTGCAAGGTCAGAAAC
TAGCCAAGGTCCTTCTCAGGCATCTATCCTTAACTTGGTCTCTCCACAAGCTGCCTCTGATGCTCCGAGCCCCTTCTGCCTGGCGACTGGGCTC
CCTGTGTGCTTTTACCTATGACCCTTGGTCAGCAGAGTACAAGGAGCACCTAAGCTGCCTCGCCACCTCCCACTGACTGCAAACACCACATACC
TCATGCGGAACCGAATGTCTCTCCAGTAAACACCCCAGCCAGCCATTCATAAAAACCTGTCTCTGTGTGCGCTGGGACATGTCCTCCCTGTACC
CCGGGCCTGCTTTGTGTGCCAGACAGTGACTCCACAGGGATGCCAGGTCTGTGATTGCATTGCTCCTTGCAGAGGGAAGCCCTCGGTTGTCCTT
CTCTGGGAATGCTGCCACCCAGAGCAGCACACAGTGATTGCCCTGTTATTTCAGAAATTATTTTAAATAAAGATCTACCTCGTGCC
MOUSE SEQUENCE—CODING
ATGGCGGGAAGTGCTGTGGACAGCGCCAACCACCTGACCTACTTTTTTGGCAACATCACCCGGGAAGAGGCTGAAGACTACCTGGTCCAGGGAG
GCATGACCGATGGGCTCTACCTGCTACGCCAGAGCCGCAATTACCTGGGTGGTTTTGCTTTGTCGGTGGCTCACAACAGGAAGGCACACCACTA
CACCATCGAGAGGGAACTTAATGGCACCTACGCCATCTCCGGGGGCAGGGCCCATGCCAGCCCAGCAGACCTCTGCCATTACCACTCCCAGGAA
CCTGATGGCCTTATCTGCCTCCTTAAGAAGCCCTTCAACCGGAAAAAGGGAGTACAGCCCAAGACCGGACCCTTTGAGGACCTGAAGGAGAACC
TCATCAGGGAATATGTGAAACAGACCTGGAACCTTCAGGGCCAGGCTCTGGAGCAAGCCATCATCAGCCAGAAGCCCCAGCTGGAGAAGCTGAT
CGCCACCACGGCCCATGAGAAGATGCCCTGGTTCCATGGCAACATCTCCAGAGATGAATCAGAGCAGACGGTCCTCATAGGGTCAAAGACCAAT
GGAAAATTCCTGATCAGGGCCAGAGACAACAGCGGCTCCTATGCTCTGTGCCTGCTGCACGAAGGGAAAGTATTGCACTACCGCATTGACAGGG
ACAAGACCGGGAAGCTCTCCATTCCTGAGGGGAAGAAGTTTGACACCCTCTGGCAGCTAGTGGAACATTACTCTTACAAGCCAGATGGGCTACT
AAGAGTCCTCACGGTACCATGCCAAAAGATTGGTGCACAGATGGGCCACCCAGGAAGCCCAAATGCCCATCCCGTGACTTGGTCACCGGGTGGA
ATAATCTCAAGGATCAAATCCTACTCCTTCCCAAAGCCTGGCCACAAAAAGCCTGCCCCACCCCAAGGGAGCCGTCCAGAGAGCACTGTGTCCT
TCAACCCCTATGAGCCAACGGGAGGGCCCTGGGGAGACAGAGGCCTTCAGAGAGAAGCCCTGCCCATGGACACAGAGGTGTACGAGAGCCCTTA
TGCTGACCCTGAAGAGATCCGGCCCAAAGAGGTCTACCTGGACAGGAGCCTGCTGACCCTGGAGGACAATGAACTGGGCTCCGGTAACTTCGGG
ACTGTGAAAAAGGGATACTACCAAATGAAAAAAGTTGTGAAAACCGTGGCTGTGAAAATCCTGAAGAACGAGGCCAACGACCCGGCTTTGAAGG
ACGAGCTGCTGGCAGAGGCGAACGTCATGCAGCAGCTGGACAACCCCTACATTGTGCGCATGATCGGAATCTGCGAGGCGGAGTCCTGGATGCT
GGTGATGGAGATGGCGGACGTGGGGCCGCTCAACAAGTACCTGCAGCAGAACAGGCACATTAAGGATAAGAACATCATAGAGCTGGTTCACCAG
GTTTCCATGGGGATGAAGTATTTGGAAGAGAGCAACTTTGTGCACAGAGATCTGGCTGCGCGGAACGTGCTTCTGGTCACACAGCACTATGCCA
AGATCAGCGATTTCGGTCTTTCCAAAGCCCTGCGTGCTGATGAAAACTACTACAAGGCCCAGACCCACGGGAAGTGGCCCGTGAAGTGGTACGC
CCCCGAATGCATCAACTACTACAAGTTCTCCAGTAAGAGTGACGTCTCGAGCTTCGGAGTCCTGATGTGGGAAGCGTTCTCCTATGGGCAGAAG
CCCTACAGAGGGATGAAAGGGAGCGAAGTGACCGCCATGCTGGAGAAAGGAGAGCGGATGGGGTGCCCTGCAGGATGCCCGAGAGAGATGTACG
ACCTGATGAACCTGTGCTCGACTTACGATGTGGAGAACAGGCCAGGATTCACAGCTGTGGAACTGAGGCTTCGCAATTACTACTACGACGTGGT
TAACTAA
HUMAN SEQUENCE—GENOMIC
CTCTTTAGTACTTGTAAATGCCTGATGGGTTCTTCCTGCTTACTGCACAGGCAAAACCAATTTATTGAGACCACACCATCACAATAAAAGAGTT
TAATTGACACAAGGCCGGCCACACCACATGAGAGATGAAGTTACTACTCAAATCAATCTCGGAAAATTTGGAGGCTAGGATTTTTCAAAGATAG
TATGGCAGGCTAAGGAGTCTGTTTCTGGGTGGGAGCACATGACTGGTCCGTAGGGTTGGTGGGGTCAGGGAGGTCAGTGGGGTCAGCAGGGCCA
GCAGGGTCAGCAGGGTTGGTGGATCCAGGTGAAGCCATTGGTTGTCAGAAATTCAAAAACCTAAAAGGTATCTCAAAAAGCGAATCTTAGTTTC
TGCAATGGTGATGTTATCTGCAGGAGTAATTGGGAAACTTGCAAAACTTGTGACCTCTAAATAATAGCTGGTAATCATTTATGTCTACACCTTA
GCAGAATTCAGGCTCCTCACACCCTCCTAACCTGGTGGCCTTTCACAAGCTTTACAAAGGTGGTTTAATTTGGGGAAAGGACTATTGTCATTTA
AACTATAAACTAAATGTCTCCCCAGGATAACTTAGCTCAGACCCAAGAATGGTTAAGGGCAGTTTGGAGGCTAATGGCAAGATGAAATTTGGTT
AGATCAGATACTTTCCACTGTCATAATTTTCCCACTGTTATAATTTTTGAAAAGGTGGTTTTAAGTTAGAATGGCAATCATTAAAAAGTCAGGA
AACAACAGGTGTGGAGAGGATGTGGAGAAATAGGAACACTTTTACACTGTTGGTGGAACTGTAAACTAGTTCAACCATTGTGGAAGTCAGTGTG
GCGATTCCTCAGGGATCTAGAACTAGAAATACCATTTGACCCAGCCATCCCATTACTGGGTATATACCCAAAGGACTATAAATCATGCTGCTAT
AAAGACACATGTACACGTATGTTTATTGCGGCACTATTCACAATAGCAAATACTTGGAACCAACCCAAATGTCCAACAATGATAGACTGGATTA
AGAAAATGTGGCACATATACACCATGGAATACTATGCAACCATAAAAAATGATGAGTTCATGTCCTTTGTAGGGACATGGATGAAATTGGAAAT
CATCATTCTCAGTAAACTATCGCAAGAACAAAAAACCAAACACCGCATATTCTCACTCATAGGTGGGAATTGAGCAATGAGAACACATGGACAC
AGGAAGGGGAACATCACACTCTGGGGACTGTTGTGGGGTGCGGGGAGCGGGGAGGGATAGCTTTAGGAGATATACCTAATGCTAAATGACGAGT
TAATGGGTGCAGCACACCAGCATGGCACATGTATACATATGTAACTAACCTGCACATTGTGCACATGTACCCTAAAACTTAAAGTATAATAAAA
TAAAATAAAATAAAATAAAATAAAATAAAATAAAATAAAATAAAATAAAGAAAAGGTAGTTTTATAAAGATAAATATACAGATCAATGAAATAG
AATTGGAAGTCTGGAAATAAACCTTTACATTGATTTTCAAAAAGTGTGTCAACTCAATTAGGACAGAATAATTGTTTTAACAACTACTGTTGAA
CTGGTACAACTGGAAATCTGCATGCAAAAAAAATGAAATTGGATCTTTGCCTCATATTGTACATGAAAATTAACTCAAAATGGATGTTGGGCAT
AAATGTAAGAGTTAAAACTATAAAATTCCTAGAGGAAATCATAGGAGTAAATCTTCATGACCTTAGATAGTAGTTTCTTAGCTACATCACCAAA
AGCATAAACAACAAAAGAAAAAAGTCGATAAATTGAACTTCATCAAAATTAAAAACTTTTATTCTGCAAATGATACCATCACAAAAGTGAAAAG
ACAACTCTCAGAACAGGAGAAAATATTGGGAAATTCTATATCTAATAAGAGACTTGTATTCAAAATATGTAAAGAACTCTTACAACTTAATGAT
ACAAAGATAAGTGACTCAATTGAAAACTGGGCAAATGATCTGAATAGACACTTCTCCAAAAATATGCAAATGACAAAGAAGCACATGAAAAGAT
TCTCAGCATTGTTAGTCATAAATGAAATGAAAATGAAGTCATCAGATACCACTTCACGCCCACTAGGATGGCTATAATCAAAACGACAGACAAT
AACAAGAGTTGACAAAGATGTGGAAAAATTGGAACCCTCCTGTTTTGCTGGTGGAAACCTGAAAATGGTGAAGCCACTTTTGAAAACAGTTATC
AGTTCCTCAAAATGACAAACATAGATTTACTATATCACTCAGCAATTCCACTCCTAGTTATATATTCAAGAAAAAAGGAAAATATACACCCACA
CAAAAACTTGTATAGGAATGGTTGATGCAAAATTATTTATAGTAGCCAAAGAGCAGAAACCGATGAGTGGATAAATAAAATGTGGCCTATCCAT
ACAATGGAATATTATTTGGTACTAAAAATGTAAAACACTGATGTATGCTATAACATGAATGAAAACATTATGCTAAGTAAATAAGCCAGTCACA
AAGAGCACATATTACATCATGCAATTTATATGAAATGTCCAGAATAGGCATGTCTACAGAGACAAAAAGTAGATGGGAGGTTTTCCTAGGGCCT
TGGATGGGGAGGAGCCAAAGGATTGTGTGGGATGGGAAGCATGAGAAGTGATATGGGTATGGGGTTTTGTTTTGTGGTTTAAAAAAGTTCTTCC
AGGTGCAGTGGCTCACATCTGTAATCCTAGCACTTAAGGATGCAGTGGCAGGAGTATAGTGTGAGCCCAGGAGTTTGAGACCTGCCTGGGCAAT
ATAAAGAGACCCCATTCTTCACAAAAAAGAAAAAAAGTGCTTAAAAAAGTTCTAATATTGATAGTGATGATGGTTGTACAACTTTGTGAATACA
CTTAAAACAATTTAATTGTATACTTTCAACAAGTAAACTGGATGTTATGTGAATTATATCTTTTGAAAACTTAAAAATGGAACCGCACTTTGAA
AAATATAAAAAAGAAAAAGAATGAAAAGATCTTGGTATGAGAAAAACCATCTAATTTTCACTACAGTGAAAAATAACAGCTGAACTACCTTCTA
ATTATATAATTATATTTTAAAAGAGTACTCTGTTAAGCAAATGATTAGCTACCCATTGAAATAGATGAAAGGGAGGAGTTATTTTTGAGCATAA
GTTTTTTCCATCCTTTTGAATCTCCAGTGGCTTGGGTCATGTCTGGCAAGTAAAGGACCCTAATTCAATTATTGAATTGTTTGCTCCCTGGAAT
TTCACAAAAAAATAAGGAATCAGGTACACAGAGGAAACTTGACTTGCTCAGAGCTGCACACCTACAAAAAGTAGCAGAGCTGAGACTACTATGG
CCCGTGCTTCCTGACCCGTCGCCAGTGCACCTACCGCTGGGTTTAAACACATGTGATCACACTTCAGACACTATCTTTGGTCTTTAAGTAACTT
TTCTGTTTTGTTTGTTCAAAAGATTACTAACAAAAGAAAAATGAGGAAGTTTACCATTTGTAGGTGAAAAGTCACTTTTATATCTGGTTGAATT
GGTTAGGGAATACCAGCAGAAGGAAGTTGTTCCTCTGGCTACATCCTGTGTCATTTCAGCAACTGGATGAATGTGATCAGAAGGTGAAAATCAC
TGAGAGAAGCCACTCACCTCCATCATGCTGGTCTATCAAAATTAACTTCCCTTCATAGGACACAATAGGGTTGTTACTTTTTTTTTAAAGGTAT
ACCTACATATGAAAATAATTAATTTTATCTAAATTTAATGTAATATGCATAAAAATACACCTGATGCTGTCCTAACTATGCAATATTCTCCTTT
GTGTGTCAATAAAAGTGTATTATTTCAATACATGGAGTTGGTTATTATTTTACTTGCAACCTAAAGCACCTTAACAGATATATCATGTGTAACT
TGTGTATTTTTTTAATTTAGAAGAGACAAAAATGAGGACCTTACATTCTAAATATTAAAATGTGCAATCACATGAGTCCAGGAGTTTGAGACCA
GCCTGGCAACGTAGTAAAACCCCATCTCTACTAAAAATACAAAAAGTCCCAGCTACTCAGGAGGCTGAGGTGGGGGAATCACTTGAGCTGGGGA
AGCCAAGGCTGCAGTGAGCTGTGATTGCGCCACTGCACTCCAGTCTGAGTGACAGAGTAAGACCCTGTCACAAAAAAAAAAAAGTACAGCGTAT
ATTCTATAGTAAAAAACATGTGGGCCATTACATTCTGAAAGAACATAGTGAAACATTTACAGAATATCAAGGCCTCACCATCTAGAAGAAAAGA
ATCCTTACTTTCCTCATCTTTCTACCTCAAGAAAAAAGGGAAGCGGGAAGAAACAAAAGTGCTAAAGTTACAAGTGGTAACCAAACAAGAAAAG
GAGAACCGCTTAACAAAATGCACTTTGAAAAGCGCTGGCTCATATAAAAACCAGAAGTTTGAGAGTATCACAATGAGGCCACACTCCAACCTCC
CAGAAACCATATATTGTGCGTCTACAAAATCCTGAACAATTCTCACTCAGATCTTGAGAGAAGAAGGGGCCATTGTTTATCCTTAGTTTTTCTC
ATTTTTGACTGTGGAGTAGTGAAAATGGCTGCAGTGGAGATTAGTGCAAAAGGAAATAGCAGCCATGTGGCATGATTTTGTCAGAGTGTGAGGA
CTCCAACGACCCACATTAATTGGGGGAATGAGCTGAAACTCACAGATGCCCCCAACCTCGCTGGACAGAGTAAGCTCAGCCTTCTCCCTCCCTC
AATGAAGTGCGAAGCCTGCATGTTCCTCACAGTCTCACATTGGAAGGAGGGGCCCAGTTTGTGCAACACAACTCCAGCTAAGCCCACCCCCACC
ATTCCCACGCTCAAAGGTTATAGAAAATCAAGCTCAAAAGGAATATTAAGCCGTCATATGTAATCCAGAAAAGGGGTAAGAAAAGGCTCTGACA
TGAGGATTTAGGAGCTGCCACACACAAAGGGGCAGAGCAAAGTGAAAGTCTGAAGAAAGCTGCCAAGTATGGACAAGATGAAATCAATGAACTA
GCAGCTATGCAAACCTTAAAAGAAAATAACATGCACAAATTAAAAATAAAATCCAGTCTGAAAGAATTTTTAAGTGCACAGGCTACAAAATTTG
ATAAGAGCCTGCATTCAATTTTTTAAAAAACTCAAGAACCGATTGAATAACCAAGAGAAAATAACTAAAAAGAGTGAGATAGAGAGAACCAGAG
ACAAAGAAATAATTTGAGGACCAATAAACCTACCTACAGGTAAATTAGAAATAACAAGAAATAAAGCAGACCCAGCTAAACATTTAGTTCCCGA
AAAAAAAGAAAGGGAGAGAGAGAAAATCACTGTGAACCAGGAAGAAAAAACAAAGTAGATTACTACAATCAGAGAAAAGATAATAGACTTAGAT
GATAAAAAGTTAATTAATATCCTTGAAGAAAAGCACTTAACAAATAAAAAGGTATTTAAAATCACACAAGAAAAAATTCTTTGAATGAAGATCT
AAATCTGCGCATTACAATGGTGGATACACAGCATTATATATTTGTTAAACACCATACAATATACAACACCAAGAGGGAACCTCAATGCAAACTT
TGGGCTTTGGGTGATTATGATGTGCATTAAAGAGTGCATTGTGTTTAAGAAATAAAAGATGGCCGGGTGTGGTGACTCCTGCCATCACTTTGGG
AGGCCAAGGCAGGATGATCACCTGAGGTCAGGAGTTCGAGACCAGCCTGGCCAACATGGTGAAACTCTGTCTCTACTAAAAATACAAAAAATTA
GCTGGGCTTGGTGGCGGGCGCCTGTAATCCCAGCTGCTCAGGACGCTGAGGCAGGAGAATTGCTTGAACCTGGGAGGCAGAGGTTGCAGTGAGC
CAAGATCACGCCATTGCACACCAGCCTGGGCAACAAGAGTGAAACTCCATCTCAAAAAAAGAAAAAGAAATAAAAGATGCTGGTTGATCTGTAT
CAGTATATATCCTTGTCAAGTTTCTTAATATCTCAGTTAAAGAAAGTTTCCTCCAGCAGCCTGGAAAGAAGAGCAAGTCACCTAAAAGAGGACA
AAGGACTGGCTGTTCTCACATTTCTCCAAAACAACATTCAATGCCAAAGGGGCAAGGGAGGAAAATCAACCGAGTCTAGGCCAAAGGGAAGTAG
GATGGAGACACAATGTGCCCACCTTGATGTCACTGCAATACAACAGCAACAGAATAACATCCTGAAAGTGGAGAGCATTGATAGATGCAGTATG
TGGGATCACATCTTCACAGAGTCCATCCAGCTCATCAACAAAGAAAGTAGGAATGAGATATGGTGGTAGAAAGGTCTGGAGGTGCATAAGGTAT
CCTTTTAAATGGAGAACTAAGAAGAGACCACTGAGGGTTTATGGTTACAGAAATCAAAAATTTTAAACTTAACTATGTAAAACTCATATAACTA
GGGAAACGTGAGAGGTGAGCTGAAGTGAAGTGGAGTGAGAGAGGTAATTGGTTCATGTATCATAGCACAGTTACTGATACTGTCTGAAATTAAA
GCTCATGGTTTAGAAGAAATGACGAAAACTGCTTATTATTGTCACTTTTCTTTTCCTTTAATCAAAGTGGGGTCATTTCAGAAATGATACATTT
GTGGCAAAGAATCATTTCTATAAAATCAGCAATTCTTTAGATCCCATTTCAGAATTTTTTATTCTGTTAAATTTAAGTAGAATTAAATATCATA
TTTAACATGGATAAAATGTATAATAAATGTATTATATTTAAAATGATGCCCATTTTTATATCTATTGCCATGTGTACATATAGAAAGAGATAAT
TTTGTAAGGGTCACCTAACGGTAATGATGATTATGTCTGAAATTTGGGATGTTTCTATTTCCTTTGTGCTTTTATTCTTTTAAGGGATTGCCTA
TTAGGAATACTTGCCACTGTTACCAAAAAAACTATTTTTTTAAATTAAACACTATTTTTTAGAGCAGTTTTAGCTTCACAGTAAAAATGAGAGG
CAGGTACCGAGGTTTCCCATATACCCACTGACCTCACACATGCACAGCCTTTCCCATTATCAACAGCCCCATCAGACTGGTACATTTGTTACAA
TTGATGAACCTACTTTGACACATCATAATCACCCAAAGCCTGAACTTTACACTGATGTTCCCTCTTGGTGTTGTATATTGTATGGTGTTTAACA
AATGTATAGTGCTGTGTATCCACCATTGTAATATCATACAGCATACTTTCACAGCCCTAAAAAATCTCTGTTCTCCTTCTATTCATCCTGCCCT
CCCCACCAGCCCATGGCAGCCACTGATCTTTTTACTATCCAGATAGTTTGGCTCTTTCCAGAATATCACCTGTTGAAATTATACAGTATGTAGC
CTTTTCAGAGGGCTTCTTTCACTTGGTAATATGCATTTAGATTTCATCCATTTCTTTTCATAACTTGCTAGTGCATTTCATTTTAGTGTGGAAC
AATATTCCATTACACTGATGTACAAGAGTTTATATATCCATTCACTTACTGAATGACATCTTGTTTGTGTCCAAGTTTTGGCAATAAACTTAGT
AAATAAAGCCAGCGTAAACATTTGTGTGCAGATTTTTGTGTGAACATAAGGTTTGAACTCCTTTGGGTAAATACCAAGAAGCACAATTACTGGG
TACTACTTTAGGTAAATACCAACTACTTTGGGTAAATACCAAGAAGGACAATCCTCTTATAATTACTCATGTTGTAAGAGGATGTTTAGTTTCA
TAAGAAACTGCCAAACTGTCTTCCACAGTGGCTGTACCATCTTGCATTCACAACAGCAATGAATGAGTATTTCTATTGCTTCACATCCTCACTA
GTGTTCACTGCGGTCAGTGTTCCGAATTTGGGCCATACTGATATCTTACTGTCTTCATTTGCATTTCCCTGATGATACATAATGTGAACCATCT
TTTAGGCTAGGCATGGTGGCTCACGCCTGTAATCCCAGCATTTTGGGAGGCCAAGGCAGGCAGATCACGAGGTCAGGAGATCGAGACCATCCTG
GCGAACACGGTGAAACCCCGTCTCTACTAAAAATACAAAAAATTAGCCGGGCATGGTGGCGGGCGCCTATAGTCCCAGCTACTCGGGAGGCTGA
GGCAGGAGAATGGCGTGAGCCCAGGAGGCAGAGCTTGCGGTGAGCTGAGATGATCGGGCCACTGTACTCCAGCCTGGGCAACAGAGTGAGGCTC
CGTCTCAAAAAAAAAAAAAATTACTACATGATACTAAGTAATGCGGAAGGTGACTCAAAGGGGGAAAGGAACACAGCAGTGTAAAGGAAGGAGG
TTGTAGATGGATCTAGAATTTCCCCCTCATTTCCATCAGGTGAAAGCCTGAGAAAACTGCAATCTTTGTGCAGGCTGGGTTTGCTTTGTACACA
CTGGTCCCCTAGTGTTCATCTCCAATAATGCTGACAACTCTGAAAACCATCTGTAGACATTCTGCAGGCTCCATCTCAGGAACAATGGCTATTT
TTTCGGGTAGTTGAAGCAAAATTAAGTCCAATGATAAGCAAATATAACCATTATCAAAATCTTCCATTTATGTTTGTTAAAGCAACCTAAGTAT
GATCTGAGAAGGACTCTGTATTCTATATTTGAGTCCTTGTGGATGAACTGTAACCTAGCTTAATAGGCAGACAAGATTGAAAACCTAATTTAGG
AGTATGTGCCTTTAACAATAGCTGAGTCTTGGCCAATCCCAGTGGCCATACTTCAACCATTCATACACTGCTGAGTGTTCAAACTGTGTTCAAA
GAAGGCAAAAGCAACCTGTAACCAATCCAGTTGTTTCTCTGCCTTACCTCCAATTTCTGTATGTCACTTCCCTTTTTTTTGTCTATAAATATGT
TCTGACCATGAGGCATCCCTGGAGTCTCTGAATCCGCTGTGATTCTGGAAGCTGCCCCATTCGCAAATCATTCATTACTCAATTAAACTGCTTT
AAATTTAATTCTGCTGAAGTTTTCTTTTAACAGGTTTAGAAAAAATAATGGCAAAAATGAATGAAAATCCAATAACCCTGGAAGCAGAAAAGGC
TGGGGGCTCCAATAAGTGTAAATAGTCCCATCCCTATATTTTCTCCATGGCAATTACAATCCAGCACATTATATATATATTTTTTTGCTTCTCG
CATTTTGGCTTAGGGTAAAGCTTTTTAAAACAGGCACTGCCAACCAGTGTTATCAAGAAGGTCTGGATGCCGTTTTGTGGGAACATTTTAAAGA
GGAATGTCCAAAAGGAAAAGGGGGATGGGTTGGGAGAAGGGTATCAGGCGGGTATCTCAAAACCATTCTTAGGGCTATAGGTTTAATTTATTTG
GTTGTGGACGTCAGAGCCGTCATGGTAAGAAGGAAGCAAAGCCTTTTGTAATAATTAAAGCCTTCAGAAGCAGCGTGCCCCATTGCCCACTAGT
GCGCCGTGAAGTCTGGTGTTCACCTACAGGGTCCCTCTCAGCACTGCCCACGCCTCCCGACTGCTCCAGCACAGTAGCTTGGAGCTTGTTGGTT
TGGTGACCAAGATACACTCCAGGGAATATGCCATGCAGTGGAGTCTCTTCCCCGGCACTGCATAGCAAAAGGAAAGGGCCGCTGGGTGTCTGTG
GGTCCTGGGCAGTCACAGAAGCCACCGCGCTCGCGGGCAGGAGCGGGACCGATGCGCTCCATGTCCCGGGCAGCCCCACCTTCTCTGCCTGCGA
AGGGCCCTTGTCCGGCGGGAGGAGAGAGGCGCGCCCCACCCGGGCTCCTCTACACCTGCCGCCGCCTGGGCCGATTCCGCGGGCCTCGCCCGGC
GCTTCAGCCGATTCCCGCCCAGCTCCGGGCTCATGGGCGCGGTCAGCAGGGCGGGCCAGGGCGGCGGGGCGCGACACTGGGAGGAAGTGCGGGC
CGCCTGCCCGGGCGCGTTAAGGAAGTTGCCCAAAATGAGGAAGAGCCGCGGGCCCGGCGGCTGAGGCCACCCCGGCGGCGGCTGGAGAGCGAGG
AGGAGCGGGTGGCCCCGCGCTGCGCCCGCCCTCGCCTCACCTGGCGCAGGTAGGTGTGGCCGCGTCCCCTACCCCGCCGGGACTTTCTGGTAAG
GAGAGGAGGTTACGGGGAACGACCCGCTGCTTTCATCCCCTTTCTTGTTCTACCTTCATCGGCCGAGGTAAAAGTGCTGAAACCATGTGAATAA
AATACAGGTGGGTTCCGCCAGCTTCGCTCCTGAACCTACCCGCGCTCGGGATCCAGAAGCTGCGCCGGGAGAGAGGGGCTCAGGCCTGGGCGGA
GGGGACGGAGGTCAGACCGTGCGGAAAGTGACCCGGGCACCCCAGGGCGCCCAGGCCCCCAGGGAGCGCGGAAAGTGCGGTCGCGGCCCGGCCC
TCGGGAGACGCGGGATTGGGATCAGGCACAGCGCGAGGAAGTCGATCTTGGAGCTAGAACATTTTCCTTTGGCCATTTACACGAATCCACTGGA
AAATGCCGCAGTGTTTATCAAAGTTACTCAAAGTAGAAATGTCCAGACGTCTTATGAGCTTAGACAAATCTTTTACTACAAAAAGAAACAGCAG
TTGCATTCAAACAACAACCCTTCTGAACCACTACTAAAATTTAGCATAATTACTCTTGTGGATACATTTTCATTGTCAAGTAATTTACTTAGCC
AATGAACTTGGAGASCAAGAAAGTTTTATGTAGTAAAATGTAAATTTGAGTTAAGAGTTAAGGGTGTTTCTTTTTGTCTGTTTGTCTGTTTTTG
GCAATGTGGCTCCAAAACCTTAAGCCCACCTAAAAACTATATAAATGCAATCCATTCTTTTGTTGGAATGTTCAAGGACTAGAAAGACAATTGG
AGAAGTGAGAGTTTGAATCTTTTTTTACGTTGGAAACAGTGTTGCAAAATATTTTTTGAGTTTTGCCTGACTTAGCAAAGATTCAGTCGAACTC
AAGTAGAGTTCAAAGCATTGCAGCGTGTAGTAAAAAAAAAAAAAAAAAAAAAAAAAAGAGTTGAAGATGTTGTGCCATATCTGATTCTTGGTAT
TAAAAATAAAAAAAGGAACCATTTAATCCCTAAGAGTTCCTGGAAGAAATGGAATTGATTCTACCATCTGACTTCTTTGTCTTAGTAGGGACAA
ATTCATTGCCTTCACACAGGCAGCATTTTAATTAAATGCAACTAAATACATTGATGTCCTCCTTCTCCCCAGTGAAAGTCCGAGCAGTGCATAG
ATAGAATTATATTTTCTCAAAAAGGCTTAAATACATGTAAATTATTAAGTGTTTTAAGTGAGAAATCTTTGTCAGTTGAAATTATTTTATCAAA
TCATTTTTGTTCTGAACGGCACTTTTGTTTTAACGTATTTAGAAAACTCTTGCATTAAAACAGAAATTGATTTGTATTATCCCCTGTATTTGAA
GTGCACTTTAAAGTGTATTTGAATGAGAGATTATAATCAAATTACTTGATTTGTGTCTTATCCTCTTACTCTTCTACCTTGGATTTGAAAAGGT
TGGATTTGAACACTAGGAAAAAAGAGATTTTTCTACTGAGGGATTGTGGGAAGATTTTTTTTTAAGCCTCTGTATTTGAAGTGAAATATAAAAT
AGGATTTTATAATAGTCTTAATAACTAGAAAGTTTTAAGCTAGGATAAAAATGGGGTGTATACTTTAGGTCTTTATCTTTCCTGAGAGATGAAA
TTGGTAGAAACGTTATTTTATTGCTAGATTTCACTTTAGAAATGAATCACCCTGATCTTTGTTAGGCCCCTATTTAATATTCTAAATGCAGGTC
TAATCTCTCTGCTTTTTTTTAATGGAAGACTTCTTAGTAGAACTTCACTTATTTAGCACATTGTTTGCCCATGGTTATAATCCAATATGCTTGT
GCCAAGACGTATTTTGGAATTCAAGCTTTTTCAGAATTTGGAATTAAAATTTAGAATCTAAATTTCAGAACTGAGAAAGACATTGCTCAGAATT
TAGAAAGGTAAACATACATGTTGACCCTGCTGGTTACATGCCATTCCAATATAGTCTGGAGAGAATCCTGTAATTCCATGCATTAATATTTCTG
TACTTTTACATTTAAGAACATGAATATTCTCACTAAGTGGAATAAATAAAGACTATAAAGAGCTGTGTGTCTGTTTAGAGCAGGTGTTATCACT
ACGAGAGCTTCTCTAAGACTTACCAAAAAAAAAAAAAGTGTTGTTTTTCAGAGCTTTTTAGAGCTGCAGCTGAGGGACTGAGTCTGTATTAGAC
TCCACTGAACTACGGTTTGCTTTTAAAAGGAAAGTGCATTTATGTTGTTCAATACTTCACACTTTCCAGTAATTAGAAGGGCAGTTCCTCTCTC
TTTAGGGAGTCTTAAATAGCAAGATTTTACTGAATTTTCAATCATACCGATCAAAATAGAGGTACTTCAAGTAAGAGTGGATGTCTCATCTTTG
TAATATAATTTCAAACAGCATAGTTTCTGACAGTCTACCATTTTAGAGAAACAGAGCAGAAGTTACTTACAGGAGGGGTTACTGTCACCCTTTA
CAGCTGCAGTCTGTCCATGGAAGAAATATTATTTCCTGTTAACTTTGCAACCAGCTGGGTTTGTGTCAGTTATCTAAGCCTAAGTGATGGCTAA
TTTTTACTTTCTCAACTTTCCCTGTATAGATTTTTTTCCAGCTACCCCCTGACTTTTCTACTCTTGCTTTGCTGGTTTTAACATTTTCCACTAC
TTCTGGATTGGTTTAGCTATAACTGGGTGGAGAAAACAACAACAACAACAGGAATTTAGGCCTTTGGGGAAGAATTTGTTTTGTTTTAGAAGTC
TACACATTGAGCATTACTTTTTCTTCTAAAGCAACTAAAATCAGAAGGCAGAGTCCTAATGAGAGATTATGATCAAATTGCCTCATTTGTGTCT
TATCACCTTCCTCTTCTACTTAGATTTGAACAGGTTGGATTTGAATACTAGGAAAAAAGGGTTTTCTACTGAGGGATTGTGGGAAGATGTTTTA
AGAACTAGTCTGATTGGATTTTAGGAAAGTTCATTCATTTTGATATGATGTTATCAGGGACATTTTCTATTTGCTTATAATTTTATCTGACCAA
TATTAGCTTGTATCAATTTGAGGTTTTTTTTTCCTTTATGAATCAATTGTGCAAGGTTTGCCCTACATCTCTTAAAAGGTGGTTAAACTGGAAT
CTTAAAGACAGTTGAATTTAGCCAAGACCAAAGATATGCAAAGAAAGAATATTTTGTTTATGTTACAGTACATTTAATAAATCCAAATGGCTCC
CTTGATTCTTTTCTTTCTGGGACACCACGTATTGATTTCTGGCAGCTGGCCAAAGGCAGATGGTGGTGGAGCAAGCGAGGACAGTTAAGAATGT
TGATGATTACTGGATTACTGAGGAGTACTGTGAGCTTTCTGGTCCTCTGCTCAGAGGTGTCTAGCTCTGAAACTGGGATCCTGGCTCTGAGCTG
GAGAGGCGCTTCCAGTTACTGCTGTCCTGGAGCGCTGGAAGGGAGCATAAGTGTGAGGAGACAGTGGGCTTTTAGCCTGGGATGCAGATGAGAG
ATGTCTTGACCTGAGTGAAAACCTTGCAGGCAAGCTGCAGGTCTACCGAGCAGGGTGAGGGAGGCTCTCGACCTGGATACTGTGAACTAGGGAG
AGAAAGTAAATTCAACAGAAGGAGGGCCCAGGGCAGAGATTTAGGAATATCCACAGTTTAAATATTGCTTTACTTAAGTATACTCTCCCTGACT
ATAAGTAGCATTTGAATGTTCAACATATGACCTTTCATTTCTCCCCTCCATTGGTTTGTCTTAACTGATAAATCTTTATTTTCCCATGGTCAAC
CTGGTAGTTTATTAATATTACATCTATCAGTTCCTGTTGAGTTGACATGAAAATGTTTTGGTGTGACCTAAGTTAATTTCTCTTTTTGTTTAAT
TTTAATTTGTAATGAGTTCCTCTTTTCATTCTGCCCATCCTTTGGTACTGCTTTGAGTTATGTCCTCTAGAATTCTCACTATTTCCTTATGATT
GGCATATTTTCAAAGTTTTTTGGTGTACTAATTTTAAACAGTGTTTGTTGACACTGAGAATTCAGGACTTTGGAGTTTTAGACCACCATCACCT
TCTACTCCTTCTCACACCATCCCAGGATAATTATTCTCTCATTTGAGATTAAGAAGTTATTTGGTTTATTTTATTATGGTTATATAAATATAGT
TTACTGCTAAAGCAAGGTATATCATTCCATTTTCTATCTCGTACCCCTTTTTGCTCTTCCTGGGGTAAACCCTTGCTTTGTTTTTGTGTTTTCT
TAGTTTGCACTGTGCCTATTATCATTTCCTTCCCAACTTTCTGACATAAAGATGAAAGCCCTATTGACACAATTTTCTACAGTGTCAGATATAC
CAAGTAATCTCACGGTTCCATTTTTCTTTCTTTTTTTCTAGAACCCTCCATCTTCTTCTTGTCTAACAGTTGCTCTTCAGTCTTGCTATACATA
CAGTAATACCTGAAGAATGTTTGGACGATATTTGGTGTGGGATTTTTCTTTTTTTTTTTTCTTTTTCTTTTTTGTAGAGATGGGCTCTCGCTAT
ATTGCCCAGGCTAGTCTCAAACTCCTGGGCCCAAGCAATCCTCTTGCCTCAGCCTCCCAAAGTGCTGGGATTATAGGCATGATCCACTGTGCCC
AGCCCTATGGGGAATATTTGTTGACTAAATATGTTTTATGTAAATTGCTTATCATACTCTTTGTTGTAACATCTGTGGCATTAATATGCCTCTT
GTTAACACTCACCTACATTTTACCTGTTGTAAATCCACTGTCATAATTATTGAAAATATTTCTCCAGAGCTTTTTAATTCTATGGTTATTTATT
TATTGTAGTATAGAACTTTTATGATTTCTTCCATCAGGGTGCATTCTGAATCATGCTTAGTGAGAGATGGTTTTTAACTCCACTTTAACAGTTA
GCATCTATGTTTTACATCAGTGTCAATGTCACTTTTGTATTATATACGACACCAACATTTTATTATCTGAGTGATGAGAATGTTTATTTTTGTG
GAAATGGAGATCTCATGAGACTTTAAGCAGATTGCTGGCCTTCTCCTCGATTTCCTTGATTGATTCCTGAAGTGGAACTGCAGTGCTAAGTGGA
ATTCCCTGGGAAGCTTGCTCAAGTAAGTAAGCATGAGAAATGCTGAATGAAATAAAGTGCAGCAAGCAGGGATAAATATCATGGGCATCTTCAG
AGCCTTTGTGCAGGTAACATGCACAGAGATTCTCTGAGAGAGTCAGTCTCAGCCAGTGGTCCTACACTTCCATGACCCTGCAGTGCGTTTGTTC
TTAGAGTGGCTTTCAGGGCTGGCTCTGCCACAGCCTCTTTGGGAAACCATGCATGACCTCTTGCTGCTCCTTACCAAGCTCCTTAGGCTTTGCT
TGGTAGAGGCAGAATGTTGTGGTTGTTTTCATCTTAAATGCTAACAATATATGAATTATGTGGTGGATACACTGCCTCAAAGTGACATTTATCG
AATATTTTTGTAGCTACGGGTTGAGAGTAAGGGTCTGAGGCCCCACTGGGTTCAAGTCCCCCTTCTTTATTTGTAAGCTCTTCTTTTGTAAATG
GAGCAGTCACAGGGGGCTTCACTCTGATACCTAATGGAGACCGTGCCTGCAGTGCTCGGTACCTGGTATGTGGTTAAAACTGACATAACTGTCA
GAGCCTCTCCCAGGCGGGCTGACTCTGGGAATCTGCCCTGCAGTCTTGGAGGGAAGGCACGTCCAGATAGGTTTGATGAGCAACTACAATTGCC
AAAGTTAGTTTCTCTGACCTCCAAGATGGGAGAACTTTCTAATATTTTCTTTTAGGAAAGTTGTCCATTGCATAACTGATACACGAGTACATAT
CCTTTAGTACAGGATTCAGATACAGCGAGCAACAGAAAAGCCCTTCTTAACCTCCACCCTCAGTCCCAGCCCTGCCCCCAGTCCCACTGTTGGG
GAGCAGTTGGAGCTCTAACCTCTCTTCTTTCGTCCTGTGCATTCATGTGTGCATGTGTGCACGTCTTCAGAAGATATAATGGTGGGCTGTGTAT
GTCTTCTTAATACCTTTATCACTCTGGAACTTTCTTTTTATTAATATGTTGGGGTGCGGGGAGGTCTCCATATTCAGTGATTATTGTAATTTTT
ATGACAGTCCAGAATAGGCATGTCGATTTTTTTTAATTTTTGATATTATAAACAGTGCTGCAATGAATGCACTTTTTCTTTTCATTCTTTCCCT
CTCCTTTGCCATTTGCACTGTGTTTTCATGAAACCATCAGGTTAATCTTPATCTCAAGGAAGCCCTTCATCCCTGTCCATCCTGTTCTTCAGTC
CATCCACTTAGTTTTCCTATGATTATTTTAGCAAGCAGGTGGTTAATTTCCAAGGAATGTAGTTACTTCTTTGGGATTTTTTTAAGTGATGTAT
TAGTTTTCTGTGTATTTTATTACTTGTTTGAGTGCACCATTACCCCCGTCTCCCGCCGTATCAGTTCATCTGCTTGCATGCTTGGTGCCTCTCT
CTCCTGCTGTCAGCTTTCCTGATGTGCTGGGTATTTCCACGTGTCTGTTGGTTTTTTTTTTTTTTTAGGTACCCTAGGCATTAGTCTTTTAAGG
CTGCATTCTTGCAGCCTGTCCCACATGAGATAGGAGGAGGGCAAGACCTGGATGTGCACAGGGCAGGATGGGCCCATAACTGGTCTGTGTGGTG
TGTGGGTGGCAGGAACTCCCTGTTCTTTGTGGGTATGGCCATGCCTTCCCTATTCAAACGAAGCCACACACCTGTCTTTTCCTGCGAGGCGTCT
TCTGCTGCTTTCTTGGAGCTGGGGAGCATCTACTCTTCCCTTGTCCTCCCGCTCCTCCCTGTTCCTCCCTCAGGTCTCTGTCGCTTCTAAGGCA
CTCCACCCTTGGTCTTCTCCCACCTTGCACCCCCCACACTGCTGCCTGCCTGCACAGGGGGACTGTGATTGCCCCCTTCAGTGTGAGCCTGACC
TGCTTCATGCCTTTCAAGGAGAGCATTCATTGCCAAGCTACTCATGGAACATCTCCTCAGATCCTCAGCGTGCTATGACTTCATTTTCTGTAAT
ACTGTTTTTCATGTTTCTTCCCCTTCTCTGCATTTATAGCTGTGCATATGCTTGAGGTGGAAGGCCACCACTTCCTTTTGGTTAACAACACCAA
TCAGCTGATGGTCATGGCTACGTGAAGGAACTCACAGTGAGGGCTCAGGAAGAGCCTGGCTAGAGCAGGCTGCATCGCAGGCCTTGGCGTTAGG
TTCCCCAAATCACTGCTGGTGATCTTGTAGCTCTGTCATGAAGTCTGTCCTGCTTATTAGGATACCGAGGGTCCAAATTCATAGATTCTTAGAA
GCCTGTTTAAATATGCAGAGTATCTAATTCAACCCGATTAGAAACGGGCTCAGAAAGGTGAAGGGATTTGTCCAACCACTGAGTGATGCTGCTG
GGATTAGAACTCTCAAAACTAGGTGTTTCTAATCCAGAACCAGAATCCCTATCCACTACTCTAATAAGGCGTAAAAGAGGATCTGCTGTCCTGC
CTAATTGGTGGGCAGGTTTGGAGTCCTGTCAGCAGCTCAGGAAACCCTGGAGAGGCAGAGGGAGGACACAGGCTCAGTGTCAGACATGCCAATT
TTACTCATCACCAATGGTTACACAAAACTTGTTTTGATCTTTGTGGCTTTTGCATTGAGAAGTTAAGCATATACAATAGGAATTAGACACAATC
CCAAACCAATGAGAGACTCAAGTGAGAAGAGCTGAAGCTTTTATGCATGCCTTCCTTCCATAGAGATAAATTTACTATCCAGAGTGAACTAGAG
CCTCGTGGAAAGTGAGTGCCGAGCCCCTAGCTGTGAGTGTCTCTGTCCTTCCCACATCAGACCTCCATGTCTGTGGCTGTCCCCCTACGGGGCA
GAACTGCTGCTCTCTGCTTGTGTTGCTGTCGTGAGGGTTGGCAGTGGACAGGTGTGGACAGATCTCAGACCCGTTTCCCTCCTCTTATGGAAAT
GTTTGTGAGGCAGGTGGGACTTCAATGATTTGGTCAAGGAAAGGCAGCTTGGCAGCTGGGGAGAAGGGCTCAGGCTAGAAAGGCAAGCGTTTGT
GAGGGATGATTTCCCCAAAGAAGCCAGCAGTGTGTAAGGTATTCCCCGGAGCTTGTTTCTTCTCAGAAGACTCTGGAAGCTGTGTCAGTGTCTG
TTATGGAGGAATGGAGTATCAGTCCGATCAGGCTTTCATAACAAAGCACCACATACTGAACGGCTTGAACAGCCCAATGTGTCGCCTCCCAACT
CTGAGCCAAAGAGACTGAGGTCAGGATGTCCCAGGGTGGCTCCTTCCAAGGGTTGGGTGGGACAATGTGCCCCCAGCATCTCCTTGGCTTGTGG
GTGACCATCTCCAGCTCCACATGGCATGCTCCCTGTGTGCCTGTCTCTGGGTCCAGATCTCCTTTTCATGTAAGGACACCAGTCATGTTGGAGT
AGGGCTCACCCTAAGGACTTCAATGGGGCGTAATCACTGCTGTAAAGACCCTGTCTTTAAATAAGGTCACACTCAGAGGGGTTAGGGAGTTAGG
ACATCAACATTTGAACTTGGGGGACACAATTCAACCTGTAACAAATGTGTGAGTGAAGACTGGCACCTCCCCACGATGGCCTTTCTGTGATGCC
ATCAAGAGCACCAGGGAGCTCTCCAGATATGGAGATGGACAGATGTCTAAGGCCCCTGAAATGAGCCTGCTGTACAGATGATTCCAGAACTCTG
TCAGTGTGAAGACCAAACACAATTAAGCACATTAAAATACTTACGGTAAAGTGTTTCTTTCACTTTAGATGTGCAAAAACTTAAAGAGAGAGAG
ACTTATGAGAAGACTCAGGGAAGAAATGTAAGATAAAGGTGTTAAGGGAGACAAACTCCACAAAATTGTAAAGTGAAGCTGCTGTGGGGAGTAG
AGCCCAGACCCAGATAATATGCAGGCATGGGTCCAAGGGAGGCTTTGCACACCAGGCGGGGGAGGTGCAGGGCCATGGATGAATGGTCTTGGGA
AAACGGCAAATCGTTTGGAAAAAAAAACAAAACTTTGCTCTTTATATCAAAATAAATTCCAAGCTGAACAAAGAATAAAAATATAGCATTGAAA
TAATGAAGCTATTAAAAGAAAACTTAGGTGAGTTTTAAAATAATTGTGAAGAGGCCTGTCTAAATGTGATAAAACATATGAATTTATATTCACA
CTCATCACAAGATTGATAAGCATGATTTCCTAAAATTTTAAGCAAATCCCCATAAAATGAAAAAATATACAAATAAGTGAAAAAAATGGTAAGA
TATATTTAATTTCAATACTTGTGGTAAAACATCAATAGCTTTAATATGCAGCGAGTTTTTTCAAATCAATAAGAAAAAAGACACAGATGGGTGC
TGACAGGGCCGTTTGGTAATATCTTCTAAAATTTAAAACACAAGTGCTTTCACTCAACAACTTCACTTTTAGGAACCTATCACACATATAAATT
GACAAAAGTGGAGAAATATTCATTGCAACATTTTATTTTATAATAGCAAAAAAAAATCACTAAGGGAAGTACAGTTGATTCATATGTTGGAATC
CTATGCAGTCTTCTAAAAAATGTGGAATTTATTTCATACACCAAATATAACAATGTCCAAGTTCTACTAGTGAAAAAGCAAGATGTAACATATC
ATTGTATTTGTTTAAAAAACTCATGCTGGGTTGGGTGCAATGACTCATGCCTGTAATTCCAGCACTTTGGGAAGCCAAGGTGGGTGGATTGCTT
GAGCTCAGGAGTTTGAAGCCAGCCTGGGCAATATGGCAAAACCCCACCTCTATGACAAATACCAAAAAAAAAAAAAACTAGCCTGGACTGGTGG
TGTGCACCTGTAGTCCCAGCTACATGGGGGCGCTGAGGTGGGGGGATCACTTGAGTCCAAGAGGTCAAGGCTGCAGTGAGCTGAGATCATGCCA
CTGCACACCAGCCTGGGTGACAGAGCAAGACCCTGTCTCAAAAATAAGTGAAAAAAAAAAAAAAGCCTAAAAATCTCCCACATGCACATAATAA
TTTAAGTGATATTGGATTGTCGTTTACTACTTATATGTGCATTTCTGCCTATACATATTCTATCCAGTGTGTACACATGTGTATGTAAATATGT
AGCATGATCTGGAAGTGTAAGCACCAAATTAAGAACAGCAGTTTTTAGGGGGAGGGGAATAGAATTGGTTTAGAGAAAAAGGTGAAATTAACAT
TTTTATTCTGCCTACTTCAGTGTGCCTGCAGTATTTACAATGATCAGGAGTGTGTACTTTTTTCAATTTAGAAAAATGATAATAATAAGGTTTG
CTTGGGGGGAAAGGGTAAGTAGGATGAGACCAGGAAGAGAAGTAACTTAGAAACACATCATGCATCATGACTGCTGGCACCATTCTGTCCTCCA
ATGAAGAGCGAGGCTTTGAGAGGTTCAGCAGCTCACCCAAAGGTGTACAGCCATGGTCTCAAACATGCTTGCACCCTGCTGTCACTCAGGTCCT
GAAGCCCAGGCCACCTCCAAGGACACTGGGGTGCTCTGGGGTGCAGCCTGCCTTCAGATGTTTCTTAAAGCCACTCCTACTCCACCCCACCCAG
TGCAGGAGCCAAGATTGAGGACCACCATTTGACAGCTAGTTCATAGGATTTCATAGCACACAAACACATCTTTAAAGGCTGATGAGGTTTCCAC
CAAATTGGGATTTATTGGTATGCTGGGTATAATGGCAGCTGTGTGTGCCTTTGACTGCAAAGACACTGGGCAGGCCAGAGTGGCTCCTCCCCAT
TTGATATGTGTGTGTGTGTGTGTGTGTCTGTGTGTGTGTGTGTCTGAGATTCAGAGAGACAGGGCTCGTGTTTTTCTGTTGCGAACAGTCCTTT
CCACGGGAACTTATATTCTCCTAAGGTTTGCCACTCTGCAGAACTTTTGGAGGAAGGCTTTAGAGTGAGAATGTCAGTGCATTCTCAGCGTGCA
ACTGTAAGACTTGCAGGAGGTGAATGCACATGAAATAATTCTCAGAGGAAGAATATGTGCACTGGTCAGATAGGAAAACTGACCTGTAACATTT
TCTACACAAGAGGATTTGAATGGTTTTCGTCTTTAAGATTTTTTTTTAACATTATGCTTAGTCCACCATGCTTTATTCTGTCAGACCATTTGTC
ATCTGCATGCTTCATGAGAAATGTTTTTTTCTCGTGAGAAGGACGTAAGCAATTTCGCTCCTGGTTGAGAGTTTGGTCCATAGTTTCTTGGTGT
CTGTGGGTTGGCTTCTGGGCCTCGCTGGCTGCAGCTGATGGTGACCAGAAGGGCTGTTCTTGTGAGTCATTAGGGGATGGGCCGCTGCTGCTAC
CTCCCCACCCCAGCCACCTCCATCACCACTGTGCCATACTCGCCCAGAAGAGCCCCTGGACTCATCGCCTGCCCCCATGTTTACACTTGTTAAT
AAAATCTCTAGAATTGTGGCTTAGACCTGTAAACATCCAGTTCCAAGAAGTGGCTCTAAAGCCCTCTCTGGGGGCAGAGTGGTTGTCAGAAGCA
GACCAGGCCTCGGGGTCTAGATTCCATCCTTCACTGGAACTGCTGGGGGCTGGTAGTACCCCATCTGCAAAATGAGGTTAAAAATACCTAACCC
TTGGGTTGCTATCAAGCTCAGAGATGCTGTATGCAAAGCACCAATCAGACTGGCTCACAAAGAATAGGCCCTCTGTCAAATGTTCATGGTTGAG
TGCGGGATGTCAAGGATAAATAGCTACTGGTTACCAGGGAATTGAAGACAGGGTTCTTTTACAAGCAAAAAGATTTTATAAGAGGGTCACAGAA
ACAGAGTGACTCTTGCCCAACAGTATCATAAGCAAATAAGCTGTGTCTAGATCCTAATCATGTGTTTAGGGCTCTGCTGGGTTTAATAAGCAAC
ACATAGGGGGTGCGTTGTCCTCCACTCTCACATCCCCAGTCACTAGACAAGGTCACTCCCCCGGGGGGCATCTTACTCAGAAGCTCTAAAAGAC
TTTGTTGCATGACTTTGGCCCTTGCTCTTTTAAAATGTCCAGTTAGAGACCATGGCATGGAGAGTTTTGTATACTCTGCTCCCAGCATGGCACC
CTCTTTGCATATTGTGGGCACTCTTAAAGTTATTTCTCATATTGTAATATATCAGATATGACTTGGATATAGTCAACTAGATATTTAGTGAAAA
ATGTATCACCCCCATTATCAAAAGAAAACCCAAAGCCTTCAAGTTTGGCCCAACTAGAAGGAATTTCTGGAAGGGGGAAGTACCAGGACCACAC
AGACTCTGGTCCTGCCGGTGTGTTTATAAGATGCAGCTCATCATGTTGGTCTCTGTCATCCCTGCTAAGGCTGTTCATTATGGAACCACTGGCC
ACATGTGGCCACTGACCGCTCAATTACTTAAAACCATTTAAAATAAATGTTGAATTTCATTTAAAATTTAAAATTCAGTTCTCAGTCACACAGG
CCAGGTGTCAGGGACCACTTGCCACACGTGGGTGGAAGGTGTGTGTCAGGCAGAGTAGAACAGGTATTTCCATTGCTGTAGCAAGGTCTGCCAG
ACACCAAGCTGCTCTCAGCCCATCCCCTGAGGAGCCCAGAAGCGATATGAACTTCCACCCACCTCTCCCCTTTCCCAGTTCTCAGTGCCACTCC
TTGCCTAAGTGCACGGTACACTCGCCACTATTTCATGTGTTGATACCTGCAGCTCCCAATTTTCTT2XAGAGGATCTGACCCTGGGAACTGATG
AGGGCACCAGGAGTGCCCTCTCCTTTGACATGAGACCTTGAGTCTTGTCCATGGGGCCCTGGCCTGGCCCTTTTCATCCCCAAGGCTGTAACAT
TCTCCTGCACAACACAACACACGTGCACACACACACACACACACACACACACACAAAATAATGTTACTTCTCAGTCACCCGGGAACTGACATCG
TTCCCAGTTATTTCCTTGCAGCAGCCCTCCAAGCGGTAACACAGCCTTGGAGAAAGCTACTAAGTCATGTATCCTTGGGGTAAGCACACTTGGT
TCAGAATAGTTTCTGAAAGAACAAACCAGGTTTTTGCTCAAGAGGGTTTTGAGGGACTGGAGAAAACCTTAGTGCTTGTTCCTTTCTCTTCGGT
TAAAAAAAAAAAATCCAACATAGTTTCCTTGAAACATTTGGAACCTGAGGTTTTGTCCATTTTGAAAAGAAGATGTAGAGAGGTAACGTGTTTC
TCACGAAGGCAAAACAAAAAAATAAGATGCCAAGCAAGTGGCCCTTGTACCTTCAAGACCAAGAAATAAGGCGTTTGTTTCTGGGAATGCCTTC
CATTTGCTCATTTTGTAAGTAATCAAGGCAGGCTGAGAGGGGAGCCTTCTGAGGCACCCCAGTGAGCAGAGGTGGGGTCCTGCCACTGGCCCCT
CGGCTCCAACCTTGCCTCACTCCAGGCGCTGGGGCAGAGATGTGTGGTTGGCCTGTCTCTGGAGTTCCTGTGCTGCTTAATTTGAGTAGAGATA
AAAGGCCACAAGAGAAGTCGTTTTGAAGGGTTPGTGAATTATAGGACTTCCTTTCATGATTCAGCCTGACTCCAGGAAGTGCAGTCACGTGCTG
CCCTCCTAGGGGAGCTTCGCCCCACCACCAGCACTCAGGCTCCACTTACTGCAAAAGGCGCGATCAAGATTCTTCTTCCTGAATTATTGTCTTA
ACAAAATTGATACGTCTAAACATTGAGTTAACACTTTCATTCACTAAAAATGTTTTCCTGAATGATGCTTTTCATGTGACTTCTTTATAGTCCC
ACGTGAAAAGCGGAATGTGCTCGGCTCTGTGCTTGTTCCCCTCTGTTTGGGAGCCTGCGCTGCTGCTCACTTCTGCAGTCAGCTCTGTTTGGAT
CAGTTTTGAACCCCAGAACCTTCCACATAGAGGTGATAATTCTAGATAATTCTAGAGCAACTGCTTCTCTATCTGGCTGTATGCTAAGCTCTCT
AGAGAGCTTTACAAGGGAGAGATTCTGAGTGTTAGGGCTGGGAAAAGAGCACTCTGTGTGATCCTAACCTGCAGCTGAAATGCAGAACCACTGT
TCTCAGCTGGAGCTTTTCATTGTTTTTTGTTTGTTTGTTTTGTTTTGTTTTGTTTTGCTTGTGAATTGAGCTTACTAATGGAAGCAGGAGCTTG
TTCTGTTTAGCGATCGTGAGAACCACCTAGGCAGCGGAGCCTGCCCTTAGAATCTGCTTCACTGGGCTGTACTGGGCTGGGCATTGGCAATTTT
AGGACACATCTAGATGTGTCCCAAGCAATCTGATATTTTGATGTTTATCTTACAGAGAAGCATGTGTTTAGAAGGCAGATAGCTGTGCTTCTGT
TCTTCTGATTTTCGACAGAGACATCAGAACCTTCCACAGTCATTTTAGTCTAAGTCCTGAAATCGTTCACTGCCAGCCCTGAGTCTAGCTGGAC
TATATGCCTCTGGTTGGACACGTCTGCCACCTCTGAGAACTGTGTGGATGTAACTCTTCTGTCTTCTGGCTCGTTGTGTTTCCAAGGAGAAGCT
TGAGGCCCAGTAATACTTAAAAATTTTTTGGACAAATTTAATCTTACATAATTATGGGGTACACAGTGATATTATATCTGTACAAGGTGGAAGG
GTTGAATCAAGCCAATTAACATACCCATCATCATAAATGCTTATCATTAATCCTCCTCTCTATCTGCAACTTTGTAGTCTTTGACTGTCATCTC
CTTATTCCATCACTCCCCAGCCCCTGGTAACCACTATTTTGTTCTCTGTTTCTATGAGTTCAATCATTTTAGATTCCACATATGAGTGAGATCA
TGAGGTATTTGTTTTTCTGTGCTTGACTTATTTCACTTTACATAACTTTCTCCAGCTTCTTCTATGTTATCACAAACAATAGGATTTCCTTCTT
TTTCATGGCTGAATAGTATTCCATTGTGTGTGTGCCACATTTTCCTTAGCCGTTTATTCCTTGGTGGACTTTTAAGTTGATTTTGTATCTTGGC
TATTGCGAATAGTGCTGCACTGAAGCATGCCAGTACAGATATCTTTTCAACATAAATTTGTAAACTTTCTTAAAACATTATGAGTTTTTTGTTT
TGTTTTGTTTTGTTTTTTGCAATTTTTTGTTTTTGGCTCATCAGCTATCATTAGTGTTAGTATATTTTATGTGTGGCTCAAGACAATTCTTCTT
CTTCCAGTGTGGCCCAGGAAAGCCAAAAGATTGCACATCCCTGCTCTAGAGGTTCCTGGATAAGATGCTCTTTTAACCCCTGTAGACAAGACTT
TTTTTATTTCTGGAAATTTTATAACTTTAAACATGTGTTTTACTCCTTTATTTTAATTCTCTATTTCAGGGACATTAATTTTATATATTTTAGA
TCACCCCTGTCTTTCTCAATTTTTCTGTTGAATTCCTGAAGTCTTTGTTAATTCCTATTTCATTTTTCTCAAGCTCCACAGATCCTGTCCCCCA
TGCTGCCTGCAGTGTTAATATCCATTCATTTTTCCCTGCTTGATATAATAGCCAACATTTCTTTTCTGGCCTTTTCTCCTGATCTCTGCTGGCT
TATCATTGTTTTTTGTTTTTTGTTTTTTCGCCCCTTCCTCCCTTTTGAGCTGTTATTTTAACCAAGTGATTATTCTGTTAAGTTCTTTGATTCC
ATATCTGTGTGTGTGGACATGCCTTCATCTGCCTTTTGGGACATTTTTCTCATGAAAGTCCTTTGCCATTTGTTTCTCCTGTTGCTTCTCTTCT
TTAATTATTTCAGCATCATTGTACAAATCCAAGGTAGGATCCTCATCTTTGAAATAGATCATGGAAGGGCCTTGGATGTGTTCCAGTTTACCAG
AATATTTCCTCCCTTCTCAATAATTTCTTGTGTCTGTGTGTGTCTGTGTGGAGAGAGAGAGAGAGAGACAGAGAGCGTCAAAGAGAGAGAATGA
ATTGGTTTAATACATATTTCTCCCCAGTGAGGCTGCCAGCCATTCAGTCTCCTCTCTTCAACACTCTAAGAAATGGTTGGCTTTCCATTGCTGT
GTATATCCTTCCACTCTGCAGGCCTTGTCTGAAGTCTACAGCAGCCAGTCCCCACTAAGGGTCTTCATGATCCCAGGGCAAACATAGCTGCATT
GGGCTCCGCTTGTGGCATGTTATTGCTCAGGATCGGGGTGCCCACTGGTTCCATTTTATTTCTTTATCTGGTTCTTGTTGCAGCGTCATTTCAG
AGAGGAGGGGGCAGTATCTCTGCCAACACCCAAGCAATCTGATATTTTGATGTTTATCTTACAGAGTGACTTAGAGTATGTGGCATTGAAACCA
ATGGTTAAAAATCAACAAATAAGCAGGAAACAACTTCAGAATGACCAAAAAAGTTCCTGGGGCACTCTTAGCTTCCACCAGCCTCTCTGATGGT
CAACCAGGCCATTTTGTTGGCTTTGATCCGTTCAGCACATTGAGTTATGGCGCGCCTCCCACGTGTCAGGAGCAGGGTGAGGTATGGGGGCTGT
CGCAGCAGCTGAGATCCTTCCCTGCACTTGACTTGTTTACGGTCAGATCGTAAGGAAGCATGGGAGCCCAGCATCAGGCTCTGCTCATGCATAA
TCTCATTTATTAGTGACAGCAACCATAGGGTTTTTCTTTGTGAAAAAAATTATAAAATACAGAACAGTGCAGTACCACCCAAATTAATAAATGT
TGTTATTTTACCATTTATGCCTTTATTTTTCCTAAAAGAACAGTCCCAATAAAGTGGAAGCTATAACCTTTTATGGATGAGGAAGTGGAGAGTA
ATCTGCTCAAGCCTGGCTAGCTGGTGAGCAGCAGGCCTGGCTCAGGCCCCTCTGGTGCCCCTGATTTAGGCTTCATGGCCTTCTCTACCCTGCA
GGAATTATGCTGGCTCAGCTGTGCAGGTTGGCGACTGCAAATATCAACCCCTCTAATAGGAAACCAGGCAGATTTTTCTCATGCTATCAGCTGA
TTTTTGGTATATGTAAATAAAACCTATGTTTTTGAGAGTAGCAGGAAACATTTTGTTAGAAAACCAAATGAATACAGGGAGATATGGCTATCAG
GAGCTGACACTGATATTATAATTTTCCAAGTACTAGTATCTGTGTATTGATTTTTACAGTAATTTGTACTTTCCCATCCAGCTTGTTTGTGACC
ACTGAGGCCCTTGGTGAATATCTTAGAAGTCACTCAAAATGGATAAAGAATGAATGTGAGTCACCCATAGCAGTCTTATGTGAGATAATATATG
GTTGATCCTCAGTATTCATGGCTTCTGAATTTGTGAATTTGCCTACTCACAATCACAGTCATTTATCTGTAATCCCCAAATCAATAACCATGAT
GCTTTCAAGACCATTTGAGGTCATACACACAGTGTATTAGTTCATTTTCCCACTGCTGATAAAGACATGCCCAAGACTGGGAAGAAAAAGAGGT
TTAATTGGAATTACAGTTCCACATGGCTGGGGAGGCCTCAGAATCATGGCAGGAGGTGAAAGGCACTTCTTACATGGTGGCAGCAAGAGAAAAT
GAGGAAGAAGCAAAAGAGGAAACCCCTGATAAATCCATGAGATCTTGTAAGACTTAATCACTATTACAAGAATAGCATGGAAAAGACTGGCCCC
CATGATTCAATTACCCCCCTTCCTTCGGTCCCTCCCATAACATGTGGGAATTCTGGGAGGTACAATTCAAGTTGAGATTTGGGTGGGGACACAG
CCAAATCATATCACCCACCCCTGGCCCCTTCAAATCTCATGTCCTCAAATTTCAAAACCAATCATGCCTTCCCAATAGTCCCCCAAAGTCTTAA
CTCATTTCAGCATTAACCCAAAAGTCCACAGTCCAAGTTTCATCTGAGACAAGGCAAGTCCCTTCCATCTCTGAGCCTGTAAAATCAAAAGCAA
GCTAGTTACTTCCTAGATACAATGGGGGTACAGGTATTGGGTAAATACACCCATTCCAACTGGGAGAAATTGTCCAAAACAAAGAGGTTACAGG
GCCAATGCAAGCTGAAATCCAGCAGTGCAGTCAATTTTTAAGCTCCAAGATCATCTCCTTTGACTCCAGGTCACACAGCCAGGTCCCATTGATG
CAAGAGATCGGTTCCCATGGTCTTGGACAGCTTCATCCCTGTGGCTTTGCAGTGTACAGCCTCCCTCATGGCTGCTTTCACAGGTTGGCATTGA
GTGTCTGTGGCTTTTCCAGGCACATGGTGCAAGCTGTCAGTGGATCTACCATTCTGAGGTCTGTAGGATGGTGACCCTCTACTCACGGCTCCAC
TAGGCAGTGCCCCAGTAGGGACTCTGTGTGGGAGCTGTGACCCCACATTTCCCTTCTGTGCTGTCCTAGCAGAGGTTATCCATGAGGGCCCTGC
CCCTGCAGCGAACTTTTGCCTCGTCATCCAGGCATTTTCATACATCTTCTGAAGTCTAGGCAGAGGTTCCCAAACCTCAATTCTTGACTTCTGT
GCACTCGCAGGCTCAACACCACGTGGAAGCTGCCAAAGCTTGGGGCTTTCACCCTCTGAAGCCACAGCCTGAGCTCTACGGTGGCCCCTTTCAG
CCACAGCTGGAGCAGCTGGGACACAGGGCACCAAGTCCCTAGGCTGCACACTGCATGAGGACCCTGGGCCCAGGCCACGAAACCACTTTTTCCT
CCTGGGCCTCCAGGTCTGTGATAGGAGCGACTGCTGTGGAGGTCTCTGACATGGCCTCGAGACATCTTCCCCATGGTCTTGGGGATTAACATTA
GGCTCATTGCTACTTATGCAAATTTCTGCAGCTGAGTTGAATTTCTCCCCAGAAAATGGATATTTCTTTTCTATTACATAGTCAGGCTGCAAAT
TTTCCAAACTTTTATGCTCTTTTTTTTTTTTTAATAAAACTGAATGACTTTAACAGTCCCCAAGTCACCTCTTGAATGCTTTGCCTGCTTAGAA
ATTTCTTCTGCCAGATACCCTAAATCATCGCTCTCAAGTTCAAAGTTTCACAGATCTCTAGGGCAGGGGCAAAATGCTACCAATCTCTTTGCTA
AAACATAACAAGAGTCACCTTTACTCCAGTTCCTAACAAGTTTCTCATCTCCATCTGAGACTGCCTCAGCCTGGACCTTATTGTTAATATCGTT
ATTAGCATTTTTGTCAAAACCATTCAACAAGTCTCTAGGAGGTTCCAAACTTTCCCACATTTTCCTGTCTTCTTCTGAACCCTCCAAACTGTTC
CAGCCTCTGCCTGTTACCCAGTTCCAAAGTCACTTCCACATTTTGGAGTATCTTTTCAGCAACACCCCACTCTTCTGGTACCAATTTACTGTAT
TTGTTTATTTTCACATTGCTGATAAAGACATATCTAAGACTGGGAAGAAAAAGAGACAGTTCCACATGGCTGGGAGGCCCCAGAATCATGGAGG
GAGGTGAAAGGCACTTCTTATATGGCAGCAGCAAGAGAAAATGAGGAAGAAGCAAAAGTGGAAACCCCTGATAAACCCATTAGATCTCATGAGA
CTTATTCACTACCATGAGAATAGCATGGGAAAGACTGGCCCCAGTAATTCAATTACCTCCCCGTGGGTCCCTCTCACAACATGTGGGAATTCTG
GGAGATACAATTCAAGTTGAGATTTGGGTGAGGACACAGCCAAACCATATCACAGAGTGGCACAAAATTTTAGCCACATGCATATTCCAGACGC
TCCAACTCCTCCTTTCACCTCATATTATAAGTAAGTGTCTTTTTCCCCAGTCTACTTAGTGCCGTATTTTTTGCATTTTTGTTCCTTTTTTCAA
ATTGTGGTGACTTTGCTGTTTAGAGTACCCCCAGCTGAAGTCCTATCTGGTGTCCCTAGGTGTAAGAAGGCTGTGCTGTGCCTTACAGGGAAAA
TATGTGGGTTAGAGAAGCCTCTGTCAGGCATGAGTCATAGTTAGTGCTGTCGGCCGTGAGTCCAATGGTAGTGCATCAACAATAGATATGAAAT
AAGGTGCCTTTACACAGAAACACATAAAACAAGTGATGCATTGATCAGTTGATGAAAATATGGTGACGGCAGGCTCTCAGGCACATAACCCCTG
AGAAGCGATGGGCTACTACTTGCTAATTCAGTTTTGGGGGTGACTTTGTGGAATGTATAACCTGAAATAATGAGAATAAACTATATTCTACTTC
TTTTACAAAGCAAGCAAATAATTTTTAAAAGCAAGTTAAATTTTACTGTTTTTGTAAGAGATATGTTTTCGAAACAGTTGAACTGTTTTATAAC
ACAGAGAAAGTCTCTTTGCCTTACATTTTATATTAGTTAATTAAGTCAGCAGATTTTTCTTGGGCTCCTGTGATGTGACTGGCACTGTTTTGGT
ACCAAAAGGGCATTGCAAAAAAAGTCCAGAAGTAGACAGATCACAGCCTTCAGGATTAAAAGCAGTCTCATCAATGTGATATTTCGTTTAAACT
TAGTAGCAAGCCCATTGAATTCTCAAAATGAGAATCTGCTAATTATCACACACTTTTCTTATGAAAGAATGATTTTTCATAGTCAATATTTCTA
AAATATAATGTCTCTTTCTCATTACCAGAGACCGATTGTATGGCAGATTTACTTGCACCTGCGCATGCTGGAGATAGGTGATGATTTAGTATAG
TGCACATCCACCAGGCGGTCCTCCTGGGTCCCAATATTTAGGCCCACAGGATGAGTCCAGGTGATATTGGGCACAGGCTCACTCAGAGGCTATC
AGACAGAAAGCTCTGCCTCACTGCACAGCAGCTTCAAGCATTGCCTTCAGTGGGAGTCTTACTTGCAGTCCACACAGCTCAGGTACAAGGCCCC
TGAGGTCCACACCTGGAGCTAATCATCTCTTTGAATAACACTGTAGACATAGCTACCAGAAAATAAAAGAAATAGAAAGGCATGATTAATACAA
GCCTCACTAGGTTCTTATTGTGAAGATCTGAGAAAGAGCTCCTCATGGCCCTGGCATGGGAGGGAAGAGTGATAACTGTAAAATACCCCTAGGC
TTTCTCCATGACAATGGTTACTCTCCAAGGGAAAACACTTTGCAAGAGCCTTGTCTCAGCTGGGAATTCCCAAGGGAATTCCTCAAGTTCCAGC
ACCCTCATCACTTTATCACTTAAAGGACAAAAAACTCATAGTCAACAGGGGTCAGCGCTTCAAGGAAATTGTTACAGAATGGTAAAGGGGTCGA
ATATCCAAGAAGACATAACAATCCTAAATGTGTATGTGCATAACAACAGAGCATCAAAATATGCAAGACAAACACTGGTAAGACTGAAAGGAGA
AGTAGACAAATCCTGTCTTATAGTTAGAGATTTTAATCTCTTTCTCACAAAAGTTGATAGACCAAATAGATAGGATAAAAATGACCTAAGCAGT
ACTGTCACTCAACTTGATCTAATTGACATTTATAAAATATTCCATTCAACAACAGAAAAATACACATTCTTCTCAAGCTCACTTGGAATATTCA
CCAAGATAGAGCACATTCTTGGAAAACATGTTAACACATTTGAAAGACTAAACGTCATACCAATTATGTTCTCAGAACACAGTATAATTAAACT
AGATATCAACAACAAAAAGATGGAGGTTTTGGGAAATTTCCCAAATATCTGGAAATCAAACAACAGACTTCTATATAACCTATGTTTCCAAGAA
GAAGTCTCCAAAATTTAAAAAAATATTTTGAAGTGCATAAAAATAAAAATATAATTTATCAAAAATTTTGGAATGTAGCAAAAGCAGTGCTTAT
AGAGAAATTTATAGAACTAAGTGCACACATTAGGTAAAAAATATATAAAGAAAATTACTTGTGATTCTACTTTTAAGAAACTACAGAAAGAAGA
GCAAATTAGGCCTAAACCAAGCAAAAGAAAAGAAATAATTAAAATGAAAGCAGAAATCATTGAAATTAAAAACAGGAAAACAGTAGAGAAAAGT
CAATAAAACCAATGTTGGTTCTTTGAAATCATCAACCAAATTGATAAACCTTTAACCAAGAAAAAAAAAAGAAAAAAATGTAGAAGATGCAATT
TACCAATATCAGAAATGAAGGAAAGGTCATAACTGTTGATCTAATGGATATAAAAAGATGATAAAGGAAGATCATGAACAACTCTGTACTCACA
AATGTGGTAACTTAGGTGAAATAAACTAATGCTTGACTGACACAAACTACTAAAACTTACAGAAGAAATAGGTAGCCTGAATAGCCCTATATCT
TTAAAATTTAAATTAATATAACCTTTAAAACAGAAATCATCAGGCCCAGATGGGTCCACTAGTGAATTCTGTCAAACACTTAAGAAAGAAAGAA
TACCAGTTTTCCATAATCTCTTCCAGAAAATAGAAATGGAGGAAACAGTTTCTAATTCATTTTATGAGTTCAGATTTCACGTAATACCAAAACC
AGTTAAGGCATTACAAGAAAACAAAGCAATATTACTTATATATACAAAAACCCTCAACAAAATGTTAGAAAATCAAACCCAACAACGTATGAAA
AGAGCTATGTACCACAGCCAAGTGGAATTTATTCCAGGTATGCAAAACAGGTTCAACATTTGAAAATAAATTAATGTGATACATCACATCAATG
GGCTAGAGAAGAAAAATCATATGACCATGTTAATGGAGGCAGAAAGGGCATTTGACAAACTCAAAACACTCTTTCATGTCAAAAAAAAAATCTT
AAACTAGGAATAGAGGAAAACTTTTGTAACTTGATAAAGAGCATTAAAAAAAAAAACCTACAGGTAACATCATACTTAATGGTGAGAGACTGAA
CACTGTCCCTCTGATATTGAAAACAAGGTAAGACTGTCCTCTCTTACCACTCTGTTCAACATCCTACTGGGAGCCCTAGCTAGTGCAAGAAAAC
AAAACAAAACTTATACAGATCAGAAAAGAAGAAATAAGACATCTTTTTTCACAGATAATATGCATGTCCATATGGAGAACTTCAAAGAATCCAT
AAAACAACTCTAATAACCAATAAGTGAGTCTACAGGATGCTAGATCAATATACAAAAGTCGGTTGCTTTCCTATATACCAGTAGTAAACTATTG
AAATTTGAAATAAGAATAATTACCATGTATAATAGCACCAAAAAGACAAAAAAAAAAAAACACAAGAAAATACTTAGGTATAAATCTAACAAAA
TATGTACAGGATCTGTATGCAGAAAACTACAAAATAATGATGGAAGAATCAAAGAAGATGTAAGTAAATGGAAAAGTAGTCCATGTTCGTACAT
TGGAAGACTCAATGTTGTTAAAATGTTCATTCTGCCAAGCTTGATCTGTAGATTCAACCCAATCCCAATCAAAATCCCAGCAAGCTGTTTTGTA
GCTAATGATGAAATGATTCTGAACTTTACTTGGGAAGGTAAAAGATCTAGAATACCCAACACAATACCCACTAAAAAGGACAAAGTTGGAGGAC
TCACACTACTCAATTTCAAGTCTTACTAGAAACAGATACAATTTCTGCCCTTATGGGACAAACAGACATTCATCAAAAATTCACATGCTAGAAA
GTAAAGGTAAATTAAGTCAAAACAGTGGTCAGTGGTACAAAAGGAAAGCATGTTAGTTTTGACTGTGGGGACAGGGACATGTTTATCCTGAGCT
CAAAGGGTAGCCTGGACAGTGGAGCATTTTTAGCAGAGAAAAGGGCCACAGCTTAGAACATGTAGGAAGGGAGCAAGGTGGGGGCATTGACAGC
CAGAGCTACTCAAAAGAGATGTGGAGGAAAAGGATGTTATGTGGTTTTGGAGACAAGTAGGCATGTGTGGGAGGCTGAGACGTGAGATGGGAGG
GATTGGCAAGGGGTTGACTTTCAGCAGTGTAGAGAAGGGAATGATCTAAGAGAGCTTGGATTTTCATCTTCATAAATGGATGGAGCTCTTGGGC
CAAGGTTCTCAAACTTGAGCCTTTAAAATACAAAACCTAGACCTTCACCCCAGAGTTTCTGATTTGGCAGGTCAGGGTGGAACCCTAAGAATTT
TCATATCCTCCATGTTTCCTGGTGATGCTGACATTGCTGGTCCAGAGACGCACATTGAGAACCCAGCTCCAGGGAACAAGAAGGAGCTCCAAGT
TAGGCAGGAAAGCCCCAAGCTTACATTTGGATGGGTTGACTATGAGCGCCTTTGCACTGCCACCTGAAGATGGTAGCTAAGAGAAGAGGCAGGC
TTCAAATATTGGCTCCACCACATACTGTGTGTTCTTCAGCAAGCCACTTGACCCTCCTGAGCCTCGATGACCAGGTCTGTGAATTGAGGATAAC
AATAGTATCTACCATTAGGGATGTTGTGCACATCAGTTGATAACCTTTGAGTCAAGTACTCATGGAATAGCTGTTTTGCATTATCCTCATCACC
TTCATCATCATGGCCATTCTGGCCAGCACTAAAGATGTACAGCCAACCTCTTCTGTGGCTGAAGTTTATCTTCTATAAAATGAGAACTTGTTTT
TCATGAAGCTGTCATTTTCACAAGTTTGGGGTGCAGGTGCAGATTGCCTACTGTTGTGTTTCTGAACACTTCCCTCGCGAGGCTCCTAACCCCA
CCCCAGTAAACTCATCAACATGACTCCTTCCAGCTTCTACAGCTCTGGAGAGGGTTCCTTTTTATCTTGCGTTTCCAGGGATGTTTCACGGCCT
TTTGTGGAGTGGGCCCTGGCTGACCTTCTGGCCCTTCTTGAGTGTGGAGGATGTCACAGACTGCAGTGGGAAATTAAAAAGCTGCTTGCTTCAC
AGAGTAAGGGAATGTTAGTAGATGCTTGAAAAGTACCCTGAGGGAGCTGGTCAGAGGTGCTCTGTAAAGTTGCATCTGTTGCTAGCACCATGAG
CAGCTGTCTGACCAGGAGGCCTGGGAACAGGGACACAGAGAGCTGGCACCCTTCTTTCCTCTGCCCTACACATTGGCCTTTCCGCCATTAAGAC
CATCTCTGCTTGACTTTTTCAGTCTCCTGGCTGGCAATTAGCATCTTGAGGTCTCCATTAAGATCTTTTCAAGGATGCTCTTCCTATTTGGTTC
AATCTTCTGGTTGAATTTGATACATGTTATGATGTAGGACTTTTTAAAAAAACATAAAACAATCAGGAAAACCTCGCTATACCCATTCCAGCAA
TTCAAGACCAAGTTCATTTTTGGTAAAAACACTTGGGAAAGGGCATTTCCAGGCTGGCTAAGCACTAAATGGCACAGCCCTGTCCACTGACTCC
CTCCTCAACTGGTCTGTGTTCTGGAGCGATCCATGTCTCAGAGCATTACTTCATTTTGAATGTGCAATTAAAAGAGGCCCCGTGCTGCTGGGGA
TCTCACACTTCTGCTTTCTGGTTTGTTTTGCATGCTGACACTTTGAGGAAAAGCCCATTTCCCTAATTACAGACATCGTCCAGCAGCAGGAAGC
ACTCTGGCTTGTTGATGCCTGTCTTGTGCTTTCTCTCTGTTTCAGCTGTTTTCCTTTTAAGATACTCCTCTCAGATTTGTGTGCTTCTTCCCTT
CCCACATTCTATACTGCCCACCGCCTGGGATTCCACATCTCCGTTTGAATCCAGGAAAGGGACGTAGGTCAAATTTGTGGGAATCAAGCATATT
TAGAAACCCTAACCTCAGGCTCTGCGCATCTGTGGGTCTCTGTAGGAGGGATACTTAGGTTTCATGCCTGCATCTGCGTTTCCTCTCCTCTGCC
GCTAACATTCTTCCACTGAGCACATGTCCATCTTGAGCTGGAAGGTTTAGCGGTTTGCCATCTTGTCTTGCCTGCAGTTATGATGCAGCTAAAG
CATCCTTCTCTCCTTCCTATAACCTATGCCACTTTGCTTTAAGCATTGAGTATATTTATGTCTCAGGTTTATGAAAGACAATTATAAAATCTCA
GGAGACCATCATAAGCTCCTGTAATATCTTGCAGTCCTGAGTCACCCACAATTATTTTTTAACTGGCTAGGAAAAAGAATGCTAGCGATTTTTT
TTTAAATAGACATTCAGTAAAACTCTAGATATTTCTTAATCCACATTTACTGTGTCCTTTTTGCTTAAGTCAGGTGTTTTGGTTTCTCTAGCAG
CTGCAGGATCCCCATAAAGATTATGCTCATAAGGTAGTGATGGAAAAGTAAAATAGAGCCTGAAAGCGGGAAACTTCAGCAAGGCACTAAGGCA
CAGACAAGAGATGTTAGGTCTGCCGGCTGTTGTCATTGCAGTCTGCTGTGGCCTCACCCCCGCCCCCCCCCCCGCCCCGCCCAGGCCTTCACAG
CTGAGTAGGAAAGGATCTTGGGATGGGTACCAGGGCAGGATCAAGGAAAGCCTGCAACTGCGCTGCCCTTCTCATCATCCATATGTGGTTTGCC
CAGATAAAAGCAATATAGTAAGATCTATCAGGCATATTCAACGTGAGTTCTTTTTTCTACCATCTTGTTGGTTGTTTGTGTTTTTCCCCAGAGA
AGAGTGAGTGATGAGGTGGCCCTGGGACATGCTAGATGGTGCAGTTCACTGGGGAGACATCCTTGGCCCCACTCAGGGTCAATATGCGCCCCAC
ACCGCTGGCTGCAACCCCACCCTGTCCTCTTCTGCTCAAAAGAGCATGCAAGGGACATTGCTAAGTGGCAATTTTGGTTGCAACCCAGTGTTCT
GTTAATTATTCACAAAAGCTAGCTCTCTTTTTAATTACCAATTGTGTGTGCGTTGCGTGTGCGTCCCTTCCCCCTTCTCTGAGCACACCTGTAT
GATGAGGCATGTACTTCTTCCTGGTGGAGAAGAACTGCAGAGATGTCTCCCAGGTTGAGCCTGCAGCCTGGCCCGGTCACCAGCACAGCACGGT
GACTTGACTGTCCTCCCTGGCTCCCTTCCCCACCCGTGGCAAGTGGCAACTTGCTTTCGCTCACACTGAACGTGGTCAACTTGCCTCCCTGTCA
CACTGTAGCACTTGAATAGGGCAATATTACATTATAGCAGAGGTTCTCAGACATTACAGTGCTTAAAAATCACCTGGGAAGCAGGCCAGGCGCA
GAGGCTCACGCCTGTAATCCCAACACTTTGGGAWGCCGAGGTGGGCGGATCATGAGGTCAGGAGTTTGACACCAGCCTGGCCAACATAGTGAAA
CCCTGTCTCTACTAAAAATACAAAAAATTAGCTCGGTGTGGTGGTGTCCGCTGTAGTCCCAGCTACTCAGGAGGCTGAGGCAGGAGAATTGCTT
GAACCCAGGAGGCAGACGTTGCAGTGAGTGGAGATCGTGCCATTGCACTCCAGCTGGGGTGACAGAATAAGACTCCGTCTCAAAAAAAAAAAAA
AATCACCTAGGAAGCTGTTTAAAATTTACCCTTCTTGTCCGTATGCCCTATTTGCTGGGTCTGAGGTGGGCCCAGAACCCTGCTTTCCAGAAGG
GAGCCTGAGGGTGGGGTGCCCTGAAGACCACACTCAGGAAGAACCGGACATGTGAGCTTTCATGAGCCAGAGTCCTGCCAAGAACTAGAATCTG
CAGGTGGCTTTGGCCAGTCCCACTTAGAGGCAAGAAGACGTGAGGATAGTAGAGAACCTGGAAATCACGCAGTCCCCAGGATGAAAGTGGAACT
CTCGGACCAGATCCTTATTCTCTTACTTTCGTGAGCCATTTCTGCCTACCTGTTGTCAGGGAGGCAGGAGGTTCATTTTTCTCTCACATGGTGC
CTGACATAATGACTTGCACGTGATAGATGCTCAACAAGCATATTGAATGAATCAATCAGTTAGATTGTAAATTAAATGCACCAGATGGGCTGGT
TGTTTATTTATAACTACACAGTTAACATTTATATTAACATAATGTTAACACCAAAGCGTAATATCTGTTTTCTGTGTTAATTATGGCAAGGTGG
GCATAGAGAAGAGATTTCTGAGTGTCTTTTGCAGAGCTTGAAAATGTCTATGTGTTGGTTGAGATAGATCAGCCTTTACTTGATGCACTTTCAA
TCTTTTAAGTTCTGAAGGCACAGCAAGAGAATTTGGTGTTGAAGGTTTGGATAATGTTTTCAAGTATTTCAATATCCCTCTATTTCTAAACATT
AGTAAAAATCATGCCTCATGCTACCATCCTTAGCTTCACTTTGTGTTTCTGAATTGTGTATCTCTATATACTAAGAAGTCCCCCATGTATGCCT
GCAAATATGGGAGAGTCAACCGCTCAGCAGAATGTGCACAATGTAAGGCTGTGCCTTGGAGACCAGGCACCCTGTGCAGTGTGATGCACAGGCA
GGCCTCAGGATGGCGCCGTGGAGGGCAAGTCATACCTACTGACGAAGCCAGTGGAGTGGATGCAAGGCCAACTCAGGCCCTGCAGCTGGGGTTC
TGTTCCTCAGTGTTGAGGCCACAGGTCTGTGTCCACATGTTTTCTTTTGTACAGTGGGATTCTGCCGCACAAGGCAGTGCCCCCAGCCCTTTGC
AGATGAGTCAAACATGCTTGTTTGCAGAGCCTGGTGCTGGGTGGGGTGGTCCCATCACATCACCTCTTCCTTGGTCATTTCTCTGTAATTAATC
AGCCAGTCCCTGACCAAACCAGCAAGATCAATTCTCCCTCTGGTTGGACTCATTCCTCTTTTTTTTTTTTTTTTTTTTTTGAGATGCAGTCTCG
CTCTGTCGCCCAGGCTCGAGTGCAGTGGCATGATTTCTGCTCACTGCAAACTCCGCCTCCCAGGTTCATGCCATTCTCCTGCCTCAGCCTCCCA
AGTAGCTGGGACTACAGGCGCCCACCACCACCCCTGGCTAATTTTTTGTATTTTTAGTAGAGACCGGGTTTCACCCTCTTAGCCAGGATGGTCT
CAATCTCCTGACCTTGTGATCCACCCGCCTCAGCCTCCCAAAGTGCTGGGATTACAAGCGTGAGCCACCACACCCGGCCTCCTCTTTCTTATGA
AAATTAACCCACAGATGAAGTTTCCTTTCACCCAAGTCTTGACAATTTTCTCAGCTTCAAGCTGCAGAGGAAATTCCTAGGCACATCTTAACAG
GGCTGCTTTGCATTGAGGGGGCTGTGTGGTTTGAGCGTGATAAGCCTGGATATCTTTCTGCACTATGCTGATTTTTGTTCCCTTAAATGGTATG
GATTTGGCAACAATTGTTTGCAGGTGATCTTTTCCCAGAGAACCACTTATTTCCCCCACTGTCTCTGTATGATAGGAGGGCTCTAATGGCCCAG
GGATGTGAAAGTGAGACACCTTCAATCGCTAAGGACTGCTGGAAATGTGAACTACAGGAAGTTGAGGGGGCTGGAGGGGAGGAGGCAGCACCTG
CCGGAGCCATGCTTCAGTGGGTCTCAAGAAAAGCAGAAGCCTGGAATCTCAGGGCTGAGGCCCCTTGATAACTAGGACAGATGGGTTAATAAAA
AGGGATACAGAGACATAGGCTACCTCTTCTGGTTCAATGCCAAGCTAGAAAATCCTGAAATGAACACAGATTTATGAGATAGGAGTAATATATT
TAAGAACACAGTCTTACATAGACATAACTATTTTCCCAAAACATCCAAGGTAAATTTATATATCTTTATGGATGAGGTTGGGTCTTTTCAGGGA
AGGTTGGGGAGACGGAAGGGAAATTACTTATTATCAGTCCTCATGATTCATGGATTCCATATCTCCGCATTCATCTATTCATTTAAATCTGTTT
GCAACCCCAAAATCAATACTCACAGCACTTTTGCAGTCATTTGTGACGTGCACAAAGCAGTGAAAAATTTAAGTCACCCAACACATACGTTGAG
GTCGCACAAGGTGACACTCTGCTTTCTTGTTTTAGCTCTTATAAACACATTTTCTTTTCTCTTGGCCCATTTAGTCCATGGTTTTTGCATTTTT
GTGCTTTTCTGGGGTGATTTTGCTATTTAAAAATGGCCCCAAACACAATGCTAAAGTACTTACTGTCTGGTGTCTCTAAGTGCAAGAAGGTTGT
GATGTGCTTTACAGAGAAAATACGTGTGTTAGGGAAGCTACTTTCAGGCATGAGTTATTGTGCTGTTGACCATGAGTTCAAGGTTAATGAATCA
ACAATATATATGAAATAAATATCTTTAAATAGAAATACACATAAAACAAGGTTCTGTATTGATTGATTGACAAAAAAGTTGTAACCAGAGGATC
ACAAAAACCTAACACCATATTTCCCCAGGAGCAACTGTTCAGGATTTGCAAATTCAGCTTTCATGGTGATTTTATAGAGCATAACTATTGTGAA
TAACAAGAAGCAACTGTATTTTGTTTCATGCATAAGATGGAAAGAGAACAGAACCAGAAGTATGATATCGATGAAATGGAGAGAGGAGAAAGAA
AAGAAAGAAATGACACTATGAAGATTTATTAGTTTATTAGTTACTAGTGATAATAAATTAATCAGAACTTTGGCCTCTGCCTTTGATCCTGGTT
CCCATAATCTCTAAGTGCCAGGCTCAGATAATGTGGCCCTTGCAGGGTTTGGGCAGCTAGTCCTCTAACCATTTTCTACAGGTAAACGTGGACA
TGAAAGAAGCCAGGGTGAGGCAGGCAGCTTCCAGAAGCACACACTGGGCTCAGCTCCTTCCTTCCTAACCTCTATTTTAGCTCCACGTACCTCT
TCAATACCAGAAAGTCTTCTTCTGATTCTGCCAGAGCCCAGCTCCTAGAACCTAGCTCATACACTCACCAGCCTTTTTTGTCAACAATTCATTC
AAATATTTATTTACTTAAATATCCATTAAGCACTGTTGCTTCTCTCGCCCAACTCCATTGCACATGCTATATCAGTTATCTGTTGCTACATAAC
AGATCACTCTAAACTTAGTGGTTTAAAACAACAATAATTACTTATTATTGCTCATAATTTCTGTGGGCCAAGCATTTGAGATAGCTGGGTAGGC
TCAGGCCCTCTGTAGGGTTGTAGTCAGGTGTGGGCCAGGGCTACAGTCTCATCTGAAGGCTTGATAGAGGCTGGAAGATACTATTCCAAGGTGG
CTGCTCATACGGCTGGTCACTTACTGCTGGCTATTGGCTAGAGGCCTCAGTTCCTTACCACATGAGCCTCCCCAAAGAGCTACTTGAGCATCTT
TCCAATGAGGCATCTGGTTTCCCCAGAGTGAGCAATCCAAAAGCTGAGGAAAAGTGTTAAGGGCTTTCATGAGCTAGCTTCAGAGACCCAACAT
TGTCACTCTGCAGTCAGCCACTCTGTGTGCAAGGGATGGGAGTACCAGGAAGTGAGGATCACTGGGCACCATCTTGGAGGCTGCCTGCCATGTA
TACATTTTAACTCTCAAACAAAATTTGAAGACTGAAGACCCATACTTAAAGTAGCCTCTGTTCCAGGCAGTAGAGAACAAAATGTAAACGTACT
TACACTAATGGAGTGTCTATTCTAGTGGCTTTGGGGAATATAGACAAAAAAACAGAACTAAAACTCTAAGGTGGGACAGGGAGCTGGGAGTGTT
AGTGTCAGGTGGGCCTATAGAGCTCAGCCCCCCTTCATCATGTTGGCATTCATGCTGGCTGTTCCTCTGTAGGGGATGAAATTTTCCCCTGCCA
GAAGAATGGGCTCTGGACAGCTTGTTCAGCTGCTTGCTCAGGTGTGAGCATCTGTGCCTAATCTTCCAGTCAGACTGCCCTAGAAACATCATTT
CTTAATTAGCTGAAAGGGTATCTGAAACATTGTACTTATGGTGGCAAGCATGGATCTATTGCTCTTCATCTATAAAGTTGCTGCCTGATAAGGT
AGAACTACACAAATGATAGTTTCCCAACTAAATGCATTTTAAAGGGCAAAGGTTGTATAGTCAGCTTTAGTCCTGGCATAGGGGAAATGGACTA
TGGAAGTCTAGTTGGAAGAGAAGATTGAAACATGCCAGTAAGTGCAAAAGGATACAGTATAATTTAAATAAAACACATGGAAGACATGAGGATC
CTAGTGGGATATGATGGGCTCATCCATTTCTGCAGGCTCCAGGAGGCTGGCTGGCAGGGCTTCTTTGGTGTGGGGCAGAAAGAATCAAAGCCTG
TAGAATAAATCTGACTCCGTTCTAGATTTAACTCCTCATGGTGTATCACGTGCAAGGTGAAGCCTGACTGGCACTAGGGAGGATTTTGAGGTAA
GGAAGCATTGTCTTTGGTTCAGTCCAAGCCCTCACTGACTTTCCACCATCTTCAGGTGGGGGTGGGCATGTCCGTCTGTCTCCCAGCCTCATGC
ACACACATGTGTGCACACACATGCACACACATGCATCCCTTCTTATGAAGTGTGGGTCTGGCTGCCCGTGCTTTTTCCTTCATCTCACCATGAC
TTATCCTTCCCTCTTCTATTCAAAGTTCCCACTTGAGAGGGTCCAGTTGCCCTGGGCACCCCTCCACACTCCCCACTCATGACTGAAATTAGTT
ACAGTGGCTTTGGTATACTGTCATCCCCTTTCACCAGTGAAGAACTCTATTGCTATAGTGCCCAGGTCACACAAAGCTGGGCCGGGTCGCCAGC
GTTCCCGATTCTCACCTCTGTGTCCCTGGGTTTCCTGCTCTCTGGCCCAGTGGAACCACCTCCACGGGGTACCATTCCTTTCCAGCCTTGTCTT
TTGTTCATTGGCAATGATGTTAACAAATTAAATAGGGGAATATTTGTTCATCTAATTTATTGGTAAAAAGGCTAGAACAAGCTTATCCAACCCA
TGACCCATAGGCCACATGTGGTCCAGGATAGCTATGAATGTGGCCCAACTAAAATTTGTAAGCTTTCTTAGAACATTATGAGATTTCTTTCCCT
TCTTTTCTGTAGTTCATCAGCTATCATTAGTATTAGTATATTTTATATGTGGCCCAAGACAATCCTTCTTTTATCAGTGTGGCCCAGGGAAGCC
AAAAGATTGGACACCCCTGGGCTAGAGCAATAAAATGGGCACATCACATTTGTGTCCACAACAGCTGACACAGTGGTTTCCAGTCAGGGGTAAG
TTACCCTTAGGGAACACTTGGCAATATCTGGAGACGATGTTTGGCTGTCATGATTGGGGAATGGGGTGCTACTGGCATCTAGTGGGGAGAGGCC
AGGGATGCTGCTCAATAGTCTGTACTAGATGAGACAGCCCCCGACCCTACACACATAACAGAGAAGGATCCAGCCCAAATGTCAAGAGTGCCAA
GGTTGAGTAACCCCGAGCTAATGTGATGAATGGTGCCAGATGGAAATGGGTGGGACCATCATAAGGAGGGGGAACAGGGTAAAGGCAACCATCT
TAATTTGTAAATTGAGTGTGGGGGATGTGTTTTGACCACATGCTTTTGAAAAGGGAAGCTGTTCATTCTCCAGAAGCCCAGCGGAGATGTTGGA
TGGATGGGGCCACCCATAGCTGCATTCATGGACAGCTGGACACGGGCCCAGGAAATCTTGGCAGCGTCACTGGGGCCGGTGCACTTTTAAGAGG
AGCTTGGGTGATATGAAATTGTTTGGACAAAATGAGTGAGAGGTCTTGAAATAATGTCAAGGCAGGAAGAATGCATTGACTTGGAGTGGCTGCC
AAGAGCAAGCATTTGCAGGTTGTTTTGGGGAAGCAGAACTGGCTGTGTTCTCTTTGTCCTGTGCACAGTTGGAAGACGGGAAATGGAGGAGGAG
AGATGGGGAAGGAGAGATGGGGAATCAGATAAGAAAGTGTTTTCCAACAGAAGCATCGGCACACATTGGAGTGAGTTCCCTTCCCTTAAGAGGC
CAGGAGCTTATGTAGGAACCAAGCTCCATCTGAAGCAGCCACAGGGCTGGAGCATCTCGCAGATTAGTTGCCAGGGTTCTCTGATTCTGGCCCT
GTTAGCCTCCATCTGGACACACAGCAATTGCATGTTTAGCATTTTTATTACAAAAGCAACATACAATTTTGACCCTTGAACGTTACAGGTTTGA
ACCATGTGGGTTCACTTATATGCGGATTTTTTTTCAACAAAAATATTGGAAAAAAGTTTTGAAGGTTTTTGACAATTTTAAAAACTTGCAGATG
AACTCCATAGCCCAGAAATATAAAAAATGTTGGCCGGGCGCTGTGGCTCACGCCTCTAATCCCAGCACTTTGGGAGGCTGAGGAGGGCAGATCA
CGAGGTCAGGAGATCGAGATCATCCTGGCTAACAAGGTGAAACCCCGTCTCTACTAAACATACAAAAGTTAGCCAGGCGTGGTGGTGGGCACCT
GTAATCCCAGCTACTTGGGAGGCTGAGGCAGGAGAACAGCGTGAACTGGGGAGGCGGAGCTTGCAGTGAGCCGAGATTGTGCCACTGCACTCCA
GCCTGGGCAACAGAGCGAGACTTCATCTCAAAAAAAAAAAAAAAAAGTTAAGAAAAAGTTAAGTGTATATTGAATGCACAAAATATATGTAGTC
TATTTTTTCATTTACTACCATAAAATGTGTACAACTCTATTATTAAAATTAAAATTTATCAAAACTTACACATAAATACTTATATGCCATTCAC
AGTCGAGGAAAATGTAAATAAATATAAAGATGCAGTATTAAATCATAATTGCATAAAATTAACTGCAGTACACACTGTACTGCTGTAATAATTT
CATAGCCACTTCCTGTTGCTATTGTGATGAGCTCGAGAGTTCCAAGTACCTGCCTAAATCACTGTGTGATGCTCATCATCTCTGTGTGAGCAGT
TCATCTCTCCAGTAAATTGCATATTGCAGTAAAAAGTGATCTTTAATAAGTGATCTCTTGCAGTTCTCAAGTATTTTTCATAATGTTTAGTGCA
ATACCGTACAACCTGAATAACACCATGGGTCCCATGTGAAGGGTCACAGTGAGGCTGGAAGTGCTCCCAAGAAGTCATGACATTATAAGAAAAA
GCTGAATTGCTTGATGAGGATTGTAGATGGAGGACTGTAGCCATGGTTGCCTGCCATTTCACATGGATGAGTCATCTTGTAAACAGATGACAGT
AAATTTATGGTATTGATAAATACAGTACAGTATTGTAAACATATATTCTATTCCTTATGATTTTCTTAATGACATTTTCTTCTCTAGCTTACCT
CACTATAAGAATATAGTATATGATACATATAACATGCAAAATATGTGTTAATCAGTTATTTATGTTATAGGTAAGCCTTCTGGTCAACAGTAGA
CTATTAGTTAAGTTTTGGGGGAGTCAAAAGTTACACATGAATTTTTACTACAGGGTGGGTGGTCAGCATCCCCACATTGTTCAAGGGTCAGTTG
TATATTCACTGTGGAAATCCTAAAAAGCAAAGAGCAACATGTTTCCATCAAGCAGGATTTTGCCAGCCTCTGGTTAAACCCTGAAGCCTGAGCA
AGGCAGTGAAATCTCCAGACCTTTGGGGCTCACATTTTAGGGAAAGGGCAGGTTTGTGTTGATGCTACAAAGGTAATAGATGCTTTTGAGAAGT
TAACTATGTGCGGGGGTGGCCAAGGTAGGCCGAAGCAGCACCTGAGACTGGATCATCATGATGCAACTCTCTGAGTAAAGGCCTGAGAAGGAGG
AAGCCAAGCAGGAGGCAGGAGAGCCCTCAACCTGGAGCCAGAAGCACAGTGTGGTGGAGATAGTGAGCAAGGTGAGGTTGCAGGTCTCGGGCCT
GGTCAAGGAGAGCCTTGTGGAGGAGCAATGAAGATTTGCGATGTGATGTGGATGGAGAGGTTCCTTGTGAGCCTCACTCCACCTTCAGGGTAGG
GAATTGTCAGTAAGGAAAGGAAGGACCAGGTGAGACTTCCAATACCTGAGAGATGGCGGTGGCTTCCACGAGGGCAGCCTCAGGAGCTATGGGG
AGACATGATGGCTTGGCAATATATTTTAAAGAAAAGCTACTCAGAGGACCTGGAGACCAGCTCATCTAACCTGCTTCAAGATTCTCCATCCCTG
TGACTGCATCAGATGGAGCTCAGCTCCTGCACAAGCTCATGGCTTCCCTGCATCACTGTGAGTGGCCAATGCTTCTGGTAGAAAGCACATTCCT
TTCTGACTTGGTGAGAGGGAAGACTCCTGAAAGCAGCCAGGGTCTCATCCTCAGCAACTGTGGTGCTCAATGTCATTTCCTCAATTAGGAGAGA
CTGAGGGAGGAAGAGATTTGCTGAGGGAGTGGCAGGAGGAGTCAAAAGCCTCATCTGGAGTATGTGGAGGTCAACTTGTCTGTTACACATCCAA
GGGGGGACGTCAGATAAGCAGACATGCAGATCAGGAGCTCAGTGGAGAGAAATAAATACCCATTAACCAACAAATATTTTTGATGACTTATTCT
GCACTAGGAGCTGTTCCAGGCATGGTGATATGGCAGTGAACAAGGTCATCCTATTCTCACCTCCCAGAGCTGGCCCTCTGGTGGGAAGAACAAG
ACAATACTCAAATTCAACAGTGAATCATGATTTAAAGGGCTGCTGCCTTATGGAGATGTGCTGCAGAGAAAAATTAGGCAGGGAAGGGAGGTAT
GCGGCCCAGGACATTACAGAAGATTATTTCATGTCAAAAAACATTTTTCTACCACTAAGTGCACATTTAACTTGTGTCACAAAAAAGGTCTCAA
AAGAGCAAGGGATTTGCAGTGTTTTCAGGAGCTCACCAGACACCTGAAAACCCTCCTTGCTGAACCACTACCTGCCCAGGTGTTCACTGAGGTG
CCAGACCCAGCAGGCTGAGGGGTACAGGTGGAGTTAAGGAGGAGCTAAGTGGCCTGGTTTGTTTCTCAGTTTCGTGCTTTGGAATGAAGTAGTG
TCACACATGGAAAAAAATAACTGGGAATGTTGAATTAAGAAAAAATAGGGAGCAAGTTCTAAACACTATTTGGAAGAAAATGATCCCTGGGAAT
GCTAAAGAGAATTTGAGCCATGAGCTGTTCCTGTAGTCAGAGTTAAGCATTGTGAGTTGGCAAAAGCATCAGGCGGGAAGGACTGAGTCTGTGT
GTGCAGGAGGAATGCCTGCAGGGGAAAGTCACGGGCTCCGTCCCTGTCTCAGTTGGTGGGGGTGGCAGAGATGCCACAGCCGTGGCCAAGACTA
CGCTGGGTGAAAAGTGAGACTCTCTGGATTTGCCATACTGACTGAAAGCTGATATCTGAAAAGTGGAGCTGAGAACAGCAGCCTGGGCCACTGT
GGGCAGTGACTGGAATGGAGATGGCCATTTCCTGCCAAGGTGGCCTGAGAAAGAGGAGGTTGGAGACCAGGACAAGGCCCCGGGTGGGTGCCGG
TGGGTGCCAGTCCTGAGAAGGTATGCACAGGAACACCATGGACATTGTTTATTCCCCCAATTAAGTAGCCCAAAAATGTGCCATAAGCTGTCCT
TGCAGTGAACCAGTGATTCTGCCCAAACCATCACCACCAACATACCCAGGTCAACTCCGGCACAGGAAGTCACATGGCCAGAAAAGGGCCATCA
GCCACATCGGTGCTGACACTGAGTGCAGCACATTCTTCTGCCACCTGCTTGCGAACATATCTGCAATGCACCATCAAAGCCACACAGAGTGTCC
CTGGCGTGTGCCTGGAGGTTGCTCCGCCTCTGCAGATGCTCCCCAAGACCCGACCACAGCCTGTCTCACTTCCTTTCCATTCACTTCAATATGG
GATTCATGCCCAAGTGCCAGCACCGATGTAATGATTAGTGACAAGCCAATGAATTTTTAAAATAGTAGCTATGTTAAGATTTATATGAATTAAA
AAGTATTTGCTCTCATTTTATTTAAAATTTTGTTCCTACAAAGTTACTGTCTCAGTTTGAGCTCATGGTGCAGTCTGCTGTTCTGCAACTACAT
AAAAAGTAACCTGGGCCCTCCAGAGGCGACACAAGCATCGGGGCCAGGTACACAGTGACAGGGGTATTTGAGCTGGGTCTGTAAGGATGACTGA
GATGGCTGGGAGGAAGAGCTGCACGCCTCAGCAGAAAGCACACGTGGTGAGAACAAGAAGGGAAAGCCACAGCATCCCGGTGCAGTCCAGTGTG
GTCTGTGGACTGGGCATGTTGGAATAGGCAGGAAGGGCTGATGTGCGCTTTGAGGGAGGACCGGGCAGCTGGAGAGGGAGGCGTCAGGAAGCAG
GAGGGCAGAAGGTGAGGAGTGAAGGGCAGCAGTGGAAAGGACAGGGTTACGCCAAGCAGTGGTCATGGATATGGGCAGTAAGGAACATGCACCA
AGGGCGCCTGGAAAACGAACCTTAGAGAACGCAAGTCCTGCGGAGGACTCCTGGGTTCCTGGCTGGGACACCGCCTGGGCAGGGAAGGCAGAGA
AGATGGAGAACCTGGTCTCAGGTGGCATCAGAACCCAGCATCCGCATCACCAGGATTGCAGGAGCAGCAGCGTCTCAGCCCCTGGGTGTGTCAG
AGCCCCCAGGCCACGTGGAAGTATGAGACGTGCTGGGCTGCAGGGGAAACAATGGAAATGAGGTTGGAGGCCCGAGAAGACCCACTGCAGGAAA
AGGGTGGAGTGGGGGAAAAGCAGGCTGGGGGCAGTGGCTGGCCTGCAACAAAAGCATTGTAGAATGCAGGCGTGAGCCACCGCGCCCGGCCAAG
GCGGGCAGATCATGAGGTCAGGAGATCGCGACCATCCTGGCTAATGCGGTGAAACCCCGTCTCTTCTAAAAAAAAAAAAAAAAAAAAAACTGTA
GAAAGGGGAAAGCAGTCCAAAGAGAAGTATTTCTTCCAAGAGACTCATCACCACACTGTGTTTCCAGAGATGTCTTTCTGTTAACAGCATTCTT
AGACTCAAAAGTAAATGTAGATATACTTTAAAAAACAAAAACACCTTGCTAAGAGATGAATTTTTAGAAACTTGTTGGACAAAGAAAAGTCATT
GATAGTCAACTGCTGTGAGAGGCAAGCCAGGAAATGATGATTAATACCCTCAAATTATAATAAAACCCATGCCTCATTAGGGGCTCGCCCAGGA
GTACTGACTTGTGGATAGACACCGTCTGGGGAAAACTCTGTCGCCTGTGTCAGGCTGACGCTCTTACATTTTATAGCCCCTTGTAGGATAGTGC
ATGTTGAGAAGAAGCCGCTTNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNTTTTGCTTCTG
AAATGTATTTATTTACTGCAAGCAGTGTCTCTCCTGTGTCAGATGTCCTCGTGGGCTGGGAACCGGCACTATCTCTTTAGCACGGGCCCTGCAG
ATGGGAAGTGGCGTTGCCCAGGAAGTGGTCTGGAGAAGCGGGGAGAAGACAGCCTATGGGCCCCAGGCCTCCGGAGCCAGGGAGGGTGTCACTC
TTCATGTCTCAGGCAGTGGCTTGTTGCTATCATAGCCCCGGGCTGGAGGTGGGAAGGCCTTATGGTGACCACAAGTCACAGTTCCGTGTGGGCC
CTCAACGTGGCCTCACTTCACCTCTGGGGCTCAGCACCAGGGTGTGACTGACTGCTCGAGGCAGCGTCTCGCAGCTCAGGTGGGTGAGGTGGTG
GTAGCGTCCTCCTTGGTAATATCACTGTGATGAGGAGCAAGGCTTCTCTAACATTGTCTGGACTCAGGAGGAGCAGTTGGTTTGTTCTCATGTA
GATTCTGCCTCCTGTATGCTGCTGGGACCCTGAGGGTCAGCCCAAGAGTCCTGTTTTGCAATAGCCCTTCTGTTTGCTTCTCTTTGCGGTATGT
TTATTTTTAGAATTCTTTGTCTTTCCCCACTCCCCCCAAAAAAGAATTATTTAATTTGGTCACTTCCATCAAATGCTCTGATTTTAGCTTAGTT
AGAAGGCATGCTGAAAACTGTAATGATGATGTGAACAGAAAGACAACTGTGTGTTCGGAGGGCCAAAGAGGGAAGTGCCTTTGTTCAGAGATGG
GAGGGTTTGTCTCCACTTTGAAAGGGTTTGAGTGGTTTTAGCAAACGTTCCCTGCCACGTGGGGTCCTCACCAAAGTTCTCTGTCTCTGTCTTG
CCCAGGTGGACACCTGCGCAGGTGTGTGCCCTCCGGCCCCTGAAGCATGGCCAGCAGCCGCATGGCTGACAGCGCCAACCACCTGCCCTTCTTT
TTCGGCAACATCACCCGGGAGGAGGCAGAAGATTACCTGGTCCAGGGGGGCATGAGTGATGGGCTTTATTTGCTGCGCCAGAGCCGCAACTACC
TGGGTGGCTTCGCCCTGTCCGTGGCCCACGGGAGGAAGGCACACCACTACACCATCGAGCGGGAGCTGAATGGCACCTACGCCATCGCCGGTGG
CAGGACCCATGCCAGCCCCGCCGACCTCTGCCACTACCACTCCCAGGAGTCTGATGGCCTGGTCTGCCTCCTCAAGAAGCCCTTCAACCGGCCC
CAAGGGGTGCAGCCCAAGACTGGGCCCTTTGAGGATTTGAAGGAAAACCTCATCAGGGAATATGTGAAGCAGACATGGAACCTGCAGGTGGGCC
ACAGCTGGTCCTGCTCCCTGGGCCCAGGGGGCCCTGTGACCCCACACAGACTTCCTGGCTACATGCAACACATGTGCCTATGTGCCCTGTGTGC
CCAAATAACACAACCATCTCCTTAAGCATCAATTTTGGCCAAAAAATGCCTCTCTCTGATAAGAGCCTTTCCTCTTTCACAGCCACAGCTGAGA
GGGTGGGTCCCTTTGTGCCAGCAGTGTGGGTTTCTCCACAGCAGGGAACCACTGTTACCCAGTGCAAGGACGGCCTTTTCACAGAGGCATTGAT
AGGTGTTGATTGGTTTCTGTTTATCTGACAAGGTCCTACCATACACACTGATAGGCACTTGATTTTATGGCTTCCTAATATCTCTTGCAGAACT
TTCTGTGTCAACACTCATATAGTTGCATCACCCGTTCTTTGGCTATCTGGTATCACCTGCTATGCACCACAATTTATTTAACTAGTACCTATTG
ATGGACACATTTGCTGTTTATGAATAAACGCTGCTAACAACATTGCTCTAGACCTATCGTATACTACACTTGTATGCAACGTCTGCAGGATAAA
CTTTGAAAGGGTTGGGATTGCCACAGCAAAGGATATGTGTGTTTATTTTTTTATTTTTTATTTTTTTGAGACAGAGTCTTGCTCTGTCACCCAG
GCTGGAGTGCAGTGGCATGATCTCAGCTCAATGCAACCTCTGGCTCCAAGGTTCAAGCGATTCTACTGCCTCAGCCTCCCAAGTAGCTGGGATT
ACAGGCATGTGCCACCACACCTGGCTAATTTTTGTATTTTTAGTAGAGATGGGGTTTCACCATGCTGGCCAGGCTGGTCTCGAACTCCTGACCT
CAAGTGAACCATCCTCCTTGGCCTCCCAAAATACTGGGATTATAACCGTGAGCCACCGTGCCCGGCCATGTTATTTTTATTTATACACTTGAAA
GTACTTAAGTAATGTGTAAAGGTCTTTCTGGTTTTATTTACTGAATGAATGAGTGAATAAGTTTTGATCCTGTTTTACCATGCCAGGACTCGAG
ATAGATTGGAAATGCCTTCTGCATCATTTTCATTTTTCCTCGGTATGGTTTACTCTGCTTTGCTCTCCATGTGGCAGGGTCAGGCTCTGGAGCA
GGCCATCATCAGTCAGAAGCCTCAGCTGGAGAAGCTGATCGCTACCACAGCCCATGAAAAAATGCCTTGGTTCCATGGAAAAATCTCTCGGGAA
GAATCTGAGCAAATTGTCCTGATAGGATCAAAGACAAATGGAAAGTTCCTGTGAGTATCGTGCCTTCCCCCTCACCTCCTGCCACCAGGCCTGT
GTGGACAATTGGGAATAATTCAGCTTCCTTGTGACTGGCCCCACATGACCCTGGGAAGAGGCCATGCATTGCCATTTGACAACATGCATGCTGG
CCTGTTCTAAAGACATTTCTAACCAAAGCTATCACTTGTGACCCAGTGTTTATTTTGCCCAGTCTAACTCTAAAATGAACACTCTTTTTGGGCA
CCGAGGGATGTAATATTTAAAGAAAATGTGATTCAGCCCCATATTCTCCTTTTAATGGCAAATTATAGGTTGCATAGCACATTCTGTGGTTATC
CAACTAAAATTAAAATACTGCAGTTTTTTTACTCAAACTACCCCCTTGATCTCAGACTTTACTTCTATTTTACATGAAACAAGATGCCAACAGG
AAAGATGTTAAGGAAAAGTTCCTGGGAGTAGCATCCATGAGTGCTGAGTGCAGGTTTGAACACTTCAGCCTTAGGCCCACAGTCATTAGAGGGT
CTGTCTGAATCCTGGGTTCTGTTTGCTATCCTCAGTGGTTCAGTGTAAATCTAAATTGTAGAAGGTTCTGAGATACAGGCCAACGTGGTCAGTC
AGTTGTCCAGAGAAGGTAAAGAACTGTCTTTGGGAGGTGTCAACCTTGTTCCCTCGAGGGGGTCTGGTCAAGGTGGCTCAGTGGGGCAGTTGTC
CCTGGGGGAAATGATCCCCTGGTCACCTCTCAGTGTCCCTACAGAGATGCTTACCTTGGAAACATAACCAGAAACAGCGATGCAGCCTCATGGT
TTGGCAATTGGTGAATCCCCAGCCGACTTCGTAAACTTTTCCTAGATTTAGAAGCGTTTTCTCTGCCTGGTCATCTATTCAAAGGAATTTCCTC
AGACTTCACTGTCATACCTTGTATTTTAAAAATGATCTTAGCTGAGTGCAGTGGCACGCACCTGTAGTCCCAGCTACTCCTGGCAGGAGGATCG
CTTCATCCTCACTATGATCTCACTATCGTTTGAGGCTAGTTTGAGACCAGCCTGGGCAACATAGTGAGGCCCTGTCTCAAAAAAAGAAAAAAAG
AAAAGAAAAATTGACCACAATACTCTTAAAAAAAAAAAAAAAAAAGGAAGAAGGAAGAGTGGCCCAGGTTGCCACGCGGCAGTGCCTAGAGTGA
TGGTCTTTAGTGGGATGCAGCCCCAGCCAGCAGCAGAATGCTGTGGGGAGGGAAGACATTGCCCCTCAGTGGAGGCACCTATGGCTATTTTTGC
TGAGGTTTGGCTTCTTACCATCTCAGCAATAATTTCCAACATGAATGTCATGGGACCATGCACATAGCATCTTGGCATGTGAAGTCAACCCAAA
ATAGCATAATCCACACGATGAGGCTGGCCAACTTCTGCTGGTTAAGAAGCCTGCTCATAGCCTCTTTGGCCACGAAGCTGTTTCTTTTTTCGGA
GTTGTCTATTCCCGAAAACAGCAGTTGCATCCTTCATGGCCATGACTTTTGCTCTCTCCTTCCTTTCTGGGAGATGTATATAAACCCCATGCAG
AAGGCTGGGAGGTGCAGCTCTTGTTTGATGAACATGCCTTGTTCCCTATCTGCCCTGTACAGACTGACATCTCATGTGTCCTCACAGCACATGT
CCCTGACACTGAGGCTTATGGTTTATGCAAGAGAGTCTCAAACTTGCCGTGCTTCCTATTCAGCTGGTGTCAGAGCTAACCAGGCATGGGCCTG
TGAGAGAGGGGCCTGGGCTTCTGTGTTTTCATCCAGCACCCTCTCCTTAGTTGTCTTCTTCTCCTGGGTTCTTATTCCTGGGTTCTTATTCTCC
TGGGTTCTTATTCCACTTTATATTGCAGTTGTCCAGTTATCACCCCATAATTGCCAACCTCCCAGATAATAGCTTCCAAGGCTTTCTCCCTTGT
GTTCTCCACAGCTCAGTCCCACACCTTCCCGTGCTCCCTCCACACCTGCAGGGCTCACCTGTGGACTGATGGTGCATAGCACCCCATGCCCAGG
CCTGGCATCAGGCAGGGTATTAGTGACAGGGAAGAGCAGAGCTAAGCCTGGGCAGCAAGACCCAGCTTCAGAACCCATGTTCCCATGATGGTTG
TAGAACCTTAGACTAAGGGAAACTTTCCCCAGGCTCTGCTCCTCTGGGTCTGTGACTGTCTTGAGGATTCAATGAAAATACACACCACGTACAC
CGCACAGCACATGTGGTCATCACTGATGTAGCTTTTCTTCTCTCTTCAGTGCTCTTTCTCCATCATGAGCACCAGGAAAGTCTCCTTGTCAGGG
TATCTTATCGTGTTTTTAACAGAGAAAGTGTGAGGAAACACCTTGGAGGGGTTTGCCACCTGGCAATTTCACCTCTTGACAACCTTTCATCTCC
TACAGAACCTGGCTGGGGTTCTTGGCTGTTGCTGGGTGTGTAAACCTTCCTTGTTGCACTGTAGTGGGCGTGTTATACTGTGAGTGTTTGAATA
TGGTTGTGTATGCTTGTGGAGAGCAGGGAGAGTTTGCCAGCATACTGGATACAGTCCAAGTCTACCATGCTTCCAGCACGGGGCAGGGTCTGCA
TCCCTGAATACAACGTCTCCCAAGTCCAGGTTGCCTAGACCTTGGTCCAGATTCTTTCATCTTTAGAAGTGTTTGTTTGTGTGTTTCTTTCATG
GGAAATAGAGCTAAAAGGCTCCAGGGAACTCTTGTAAACCATCACCAGATCCTGTGGCTGCCCTCTCAGTGTGAGTGCGTCAGCTCCTGATTCA
CTGTGGGAGTCTGTCCTATATTCCCCTGTTGGGTCAGGCCAACGTTGCAGGAAGTCCCAGACCTGGTTTTGAATCATCCAAATCTGCCCTACTT
GCAGATACTTTTATAAAATGCACATTATTTGCAACGAATAGCCACAGCACAAGGTCTTTGGAGTCAGAACTAGATTCCAGTCACTGCTGGATGT
CCTCACTGAGCCAGCTGCTGTTTTTTTCCCACTGTTCTTCTCGGGCCTTCATTCAGATTCTCCTAACGAATGTGCTCATGTCTACTTTCCAATC
ATCATGTCAGCTAATCCATCCATGGACCAACATTACTGGAGTCTGTAGTTGCATGAAGCACTAAGTGCCAGGCACCATTCTGGACTTTGGGAAG
GGAGAAGTGAATCAGCAAGCACCTGCCCTCATGGACCCTGCATTTCAGTGGGAGAAAAGACGCTGAACAATAGGAAAGAACTGCATTTAAGTAA
GGGCGTAGAGCACACTGGGGTATGGATGCTGGGTCTTCTTTCTGCTGCGTGGGGAGGATGGACCTTGCTGTGAGGGAGGCCATTGAGAAGAGGC
CTGAAGGCAGCGAGGAACCATCCCCACCCAGGGAATGGCAAATTCGAATGCCCTGTGGCAGGAGCGTGCCTGGGGAGCAGGGCACCCACAGAGG
CCAGTGTCACTGGCAGAGAGTCAGTGGAGAAGGGTGGAGTTGAGGGCCAGGTTGCAGAGCACTCACGGCACTGGGAGGACCCTGCTTTGGGCAA
GTTCGGGGGGCTGTCTTTAGGAAGCAAAGGGCCACGGCCAGCTGACATTCGGTGGGCTCTGCCTGGCTGCCACATCATCGGTTTGCTGTGGGCA
GTTGTGCCTGTACATATACTTGCAAACCAAATGTGTTCATTTTTTTATCGTGATTTCAGGGCCTTCACTTTCTCATCCCTGAGCATCAGTCAGT
TTGTCTCCGCTCTGATCTCCTGTCCACCCCACCCACCGTGCTTTTACCTGGGTCCTGTACAGGCCTGTGGGCTGGCCCTTAAGAGGTTTTCAAT
GAATGCTTGTCACCTAAATCAATGAATTGTTTGCATCCATTCTCCATCTCCCAGCTCGTTTTTGCTTTCCACTTCCTGACATCTAGTAATACCA
CTGTAGTTCTGTATGGGGGCAGCTCCACACCTGTCTATGTTGGGGAGGGAATGGGGATTCTTGCACCTCTTTCTACCTCCTCTTAAATCATTAC
TGATGGACAGATGCAGGGACACAGCAATAGATATAGAAATATAAAAACATGACACTGCGCACTCTATGGTTTGTAAGATTCACTTTCTCTGGGT
AGGAGGCAGTGTGCCCTGTTAACAACATACCATTCGGGTTGTCTGGCCTCAAATGCTGGCTCCACCTTATCACCAGCTATGGGACCTTGAGCAA
GTTACTCAGTGGTCTGTTTCCTTCATTTCTTTGTCTGTAAAATGGCTTTAATGTCATCTCTGACTCTTAGAGTCATTGAGGGTTATAGAGTATC
ATAAACCCAAGGCCTCCTGCACGCTGCTTGGCACCGAACGAAGCAGCTTCACCCCACTCCTGCAGTGGCCTGGACCACACTTGGATACATCAGT
AACATACACTGAAGGTCAGGTCCTCCATCCAAACTCTTTCCAWCCCTGCATCACAGGTGTACCATGACCTCTGCAGGTGAGTCATTCTTCTGCA
GGTGAGAAAACCAGGCAGGTCCCCCTTATCCTTCCCTTCTCTTCTTCAGTCTGCAGTAGGTGTGTTTATTTCCACTCTGTCGCTATGAATAGAT
AGAGTGAGAGTCACTGGCCATAAAAGGATTCACACTAGGCTTTTGCTTTTATGAAGTGACAGACTTAAATAAAGAATAATTATTTCGGCCGGGC
ATGGTGGCTCATGCCTGTAATCCCAGCACTTTGGGAGGCTGAGGCGGGCAATCACAAGGTCTAGAGATCAAGACCATCCTGGCCAACATGGTGA
AACCCCATCTCTACTAACAGTATAAAAATTAGCTGAGTATAGTGGTGTGTGCCTGTAGTCCCAGCTACTTGGGAGGCTGAGGCAGGAGAATCTC
TTGAACCCAGGAGGCGGAGGTTGCAGTGAGCCAAGATTGTGCCACTGCACTCCAGCCTGGGCAACAGAGCAAGACTCCATCTCAAAAAATATAT
ATATTATTATTATTATTATTTCACAGAGGGGGTTGGAAGTTTATTTTTGCATTGCAAGCCATAAACTAATCAAGAGGATTTTACTAGTTTGGTG
GATAATATGGGTAAACAAAATCCCAGCGATTACTCTTCACACTTTCATCGTGAGTACCAATATGCTGTCTCCACCATTCAGATGCCAGCATTTC
AGTCTGAGAATCGCTTTACGTGGAAAGTGGCATTGGTAAGAATTGGACTAAAAGGTAAAAAAAAAAAAAAAAGAATTCCTGAAATGCATGCACC
TCCATAGCCATCACAAAATGCCTTTGCTCAAACAGACCTTGAAAACACTGAGGGTAAGGCAGCATATTTTGGAGATCAAAATCTGATTATAAAT
CCTAGGTAATGCACTTCAAAATGCAGATGATGGAAGCAGATTACGTCTGGCAGATGCTGTCCAACTCGGATCCCAGTTTTCTCTCCTCAGAACT
ATATGTGAAATCCACTTACAGTGAGAGTACACCTGAGCTAGAGGGCATTTACCACTTAAGGGGGCTAAACTTATTGTTTTTCAAACAAGAACTC
GGGTGTGGAGGAGCTTGTCCAAGGCAGGAAGGCAAAACCAGGTCCCTGATGTTGTCTGGGGCTAAGCAGAAACAGCAGCCAGCAACCAAATCCA
TCTCCAGATGGATCCATCTCCACAACCACAGGCCCCTCCCTATTCCCCATGCAAGATGCAGCACCAGGGCATGTCCATGTGAAATCTCAGAACA
CACAGAGGCGTCCATCAGCTTTCCCCTCTCTTGTGTCCTGTCCACACAGCTTACCCACACCATTCAGGAGTGACTTTGAGAAGGTAGTTACAGG
GCCTTTGCACATGTGCTTGAAGAATAACGAATATAAACAGAGGCTCTTTGTGGAGGGGCTGGGCAGTGTGGGTCAGTGGTTGTCCCTCACAGCC
GCCTGTGGCATTTCTGGAGGTGATGTTCATCACCTGGCCTTTGCTATATAAGCACTGCCCTTGTCTGGGGTCTAGCTGGCCAGAAGCGAGCACT
GGAGGGGGAGTCCTCCAGCCCACGTTTGGTGGGTGAGGGTCAGAGTGCTGCCTTGTTGCTGACGTCAGGGACCAAGGCAAGGGAGAGGAGCTGA
GTGGGGAAGGAGATCCCCTGCCCCCAGAGGCCTTGTATAGCAGCACAGTTTGCTATTTTGAGAAGCTACTCACAGCTGTCTGTGAAAAATGAAT
TTGTTAGGAAAAGGAAACAAAGCTTACTTTATAAAAATTATATATCCCTTCCCCAAAAGGGAGGATCCAGGATTGAGGGGTCCGAAGCATATAC
AATTTGAAGACTCTCTTATTTTTTTAAAAATATAAAATTAGGCATACAATAGAATGTATTTTTTGGACTAAGGAAAAAAATTCAATAAATTAGA
ATTTTTTTAAAGGCTGGTCAATACTACAAGCATACAAACTTTAGAAAAGATAATGTAATATTTTAATTAACTGCCTGACACGTCTCTATAACAC
CTTTTGTTTCCTCCATGTCCTTGACTTCACTGTGTTTGACTACATCTTAGTTCAACAATATTTAATAATATATTTGTCTATATATAGAAAAAGA
GACTATTCATTCCTTCCTCTAGGTTCACACTTCATTATTAATAATATCATGTACATTTTTAGAATCCTTGTCAAATTTGGGAGGGGAAGATAAA
GTAGAACAGAGTGAGCTTCCCTGGCTGTGACTAAAATATCTTTTGCAATCTGTACAAAACCTAAGGCCGTGCAGTCACATTGCTGGGCTCCTCT
TGGTCTGGAGGAGGCTGTGATCAACTCCTCTCTGACCCTCATCCACTTGGTTCACACAATGTGGTTTATTGCCATTGTGCACAGCCAATTCTCC
CACACACATCGCTCTGCTAAAATGTAATGGGATGTGGCCTTCATTTCATCTTATAAATGATCTTAGAATGGAAATGCTCTAAAAAGTCCATGCC
AGGCAAAATTTTAAAGATGGAAGTATTGTTTCTTTGTATTAAGTGTATTGTTTTTGGCTAAGAGTCCTTGTAAATCTTACAAAGCCAAGAACAA
TTTCAAGGTGACCTTTAAGACACATAAGTGGCTGCTCTCCAAATAGCTCGATTAATGTTAAAAATAAAAGGTAGTATTTCAGATTTGTGGATGG
AGTCGTCCAACCAGTAAGTCACCAATGCTATTTTAACTACAGTTTCTACCTTCTGACTTTAGAAAAAAAAGTTACAAAAACAGTCATTCATTCA
ACAAAATAGGCATGTTTTCAACCAGACATGCAGCAGGCATTGTGATAGGACTTGGCTACTGTGAGGAATAAGTCAGACCTTAGTCCCTGCCCTC
ACCGCACTTTGACTGCAGGGGGAAGATGACAAACGCAAGCTGATGTGCTGCAAAGTCTGCAATGCATGGCAGTAGGTCCCCTCCACCACGACAC
TGGGCCTACGCAGCATGATCTTGATTTTGTTTTCATTTACTTGTTTATAGTCCCCACCCCCACTTGTGCTGGATGCACAAGGGCAGAGATGGGG
CTGTTGCCTTCACGAGTGCAACCCAGCACCTAGAACATGGTCTTGGTGGGAAAAATCTCTCTGGGTAAAGGAACATTCTGGCTGCTAACTTGAA
TGGTGGCCACTACTTCGATAGGAGGAGGGACAACATGGTTCATCGGGCCTGTCTCAGGTGCCCAGCCTGGACTATCATCTAGTGTGTCGCTAAC
TATAGGAACTGGGAAGTGGCCTTCAGAGCAAGTGGGCTGACTGGATTTGGCAGAGCTTGACACCTCACCAGGCAGATGCAACAAAAGAGCAGGG
CAGACCCAGCCATCCCATTACTGGGTATATACCCAAAGGACTATAAATCATGCTGCTATAAAGACACATGCACACGTATGTTTATTGCGGCACT
ATTCACAATAGCAAAGACTTGGAACCAACCCAGATGTCCAACAATGATAGACTGGATTAAGAAAATGTGGCACATATACACCATGGAATACTAT
GCAGCCATAAAAAGTGATGAGTTCATGTCCTTTGTAGGGACACGGATGAAATTGGAAATCATCATTCTCAGTAAACTATCGCAAGGACAAAAAA
ACAAACACCGCATGTTCTCACTCATAGGTGGGAACTGAACATTGAGAACACATGGACACAGGAAGGGGAACATCACACTCTGGGGACTGTTGTG
GGGTGGGAGGAGCGGGGAGGGATAGCTTTAGGAGATATACCTAATGCTAAATGACAAGTTAATGGGTGCAGCACACCAGCATGGCACATGTATA
CATATGTAACTAACCTGCACATTGTGCACATGTACCCTAAAACTTAAAGTATAATAATAATAAAATAAAATAAAATAAAAAAGAAAAAAAAGAG
AGCAGGGCAGCAAGAGATTCACGGGGTAGCATGGGTATGGTGGCCAGGGCAGGCCTGGATGCCTGCAGGAGAGGGAGGGAAGTGAAGCGGTGGA
TGCCGAGTGGGTGTGCAGGAGCCCGGGCATCTACAGAACCCCAGTCCGAGCCCCAGGAGGCAGCACCATGTCATGGTCACGTGGTGCCCTGAGA
CCATTTTGGTGCCCTGAGATTCTATGGATCCTGGCAGACTTTTCCTTGATGGTGGCAGACTGCCCGGGTGGTCGAGGGATTGGCACGTGGCTGC
CCCAGGCCACACCATGAGGTGTTGAGAGAGATCCTGGCTGCTCAGCTGCCCCGAGTGTGCAGCATGGAGCAGGGGCGGCTGCTCTGACGGCTCA
CAGCAGGAGAGGCACCTGGAGAGCTGGGCTGTGATCAGGCCCAGTAGTTTGCAGATGTCTGGAGAGAAACTGAGTCAGAGAGGAAGCCCAGGAG
GTTGTGAGCCTCAGCGATCTGAGAGGCATGCATGTTGAACAGTGAGTGAGGAGGATGGGAAGCCTGCAGGCTTGGTGGGTCCTAAAGTTGCAAG
AACATCACTGTCCCCTGCAGCTAAGTGGGCCTGATGTACTCCACTCAGGCCACAGTTGCTGTACATGATCTCTTGGGACCCTAGAAGTGCTCCT
TTCCATGATAGCTCTTTAATACATTAGTTAAAACGATTCCCAGGAAGCCCTTGTTCCAAGTATGCAGGATCAGATAAAAAGCGCAGAAACCCTC
AGAGCAGGGACCGACGGTGAGTGCAGTTTGCCCTCAGAGGCCTTGGTAGTCACCCCTTCCCAACCCCCATGCCACCACTGAATCCCCATGTGGC
TTGTTCTTATATGCCCTGCAGCTGAAATTCAGGTGCCCTCCTGACCTTCCTTCCCCTGTCCATTTTGAAAAGAAATAAGCAGTGTAATCTTTGA
GTCATCCCTTTTGTACTAGCCAAGTTCCAGGTGCAGCATCACACACTCGTTATCTCCACCTCTCTTCTCCCCTTCCTTTCCCTCATGTCTGCAC
ACACTCCCATCCCCTCACTTCTTCCCCCTACAGCCCGCCTCTCTCTCTCTCACACACACACACACACACACACACACACACACACACACACACA
CTTGAGAACCGCGGCTGCATATCATCTCCTGTGGACCTGTGCACAACCTGTGGGGCCCAACTTAAAGTACTGCATCCATTCTCCGTGGTTTATC
CTAATTTAGGTTTGTTTCCTGGTCATATACAATACTCAAGTATATCTCTTTAGGAACTGTCTCAATTTCTTAGAGAAATGGGATATACGTGAAT
AAAATTTTTATGATTCCCTTATACTGAGTCCCACTTGCACTGCAAAGGATTAATAACATCTGTTTCCAAAGCAAGGCGTCACCTGCCATGCAAG
CCACCTGCTGGGGAGGGGAGGTGAAGGGTGTGAGCATTGGTGAGAGGTGTTGTTCTCATGTTCAAGGTGACAGGTATGTTATCTGGTGTCCCCA
GGCTGGGTCCCACCCAGGGCCTGCTCCTGGACGAGGGCTGTATGGCTCTGATTTGCACACCCCTGATTCTGGTGGACACAACCCTTTTTCTTAG
AAAGCCAGAATCACTGTATCTTCTAGTCCTTAGCTGCATAAAAAAATGGGGTGCAAAACTTCTCTGGACTGTCTATTCTTATTGTTCATTGGTT
TATCCTGCAGGGCCACCCATGGAGCCCGTAACTTAAGATGCACAAATGAGCTATTGAGTGAATATGTGTGTGTGTGTGTGTGTGTGGGTGTGTG
TGAGAGAGAGAGAGAGAGAGAGACAGACAGAGAAAAAGAAAGAACCATCTGGAGGGCTGGCAAAAGCACAGATGTCTGCGTTCCATCCCCTGAG
TTTTTAATTCACAGGTCTGTGTAAGGACTCTTCCATTTGCATTCCTGCCAAGTTCTCAAATGATGCTGATGCTGCTGGTCTGGGGATATGGTGT
GAGCCTGTTTGAGATACCTGTTACCTACCTGGGCTGGATTAACAACTATGATCATTTGAGATTTTCATAGTTCATTGGAGGGTCTGTAACTGCA
TTTGCAGTTGCAATTCCTGCAAAATGCCCAAATACTGTATTATTGGACTGGTTCCGATAAAGTTATCTTTGAAAATGCCTCTATTTCTAGAAAC
TTAAGTTAGATAGTAAAGAAATTCAGGCCATGAGTCAGACAGGCCCTGCCACTTAATAGCTCTGTGATTTGGACAACTTAATTTTGCTCTCTGA
GCCTCTGTTTCCTCAGTAATAAAATGGGGAGACTGATGTTTTATCTGAGTCATTATGGAGATGACTTAAGTAATGTGTATAAAGCTAAGCCAAT
GGTTCTCATGTCTCTGTGGTTCCTACATCCATGAATTCTTTTCTTTCATCAGTTCTGAAAAACATCTTGCTACTTTTTTCTTCAAATATTGCTC
TCTGTTCTTCTTGCCCATTTCTTCCTTCAGAGACTCCTATTAAATACAGTTTGACCTTCTCAATTCATCCTTTATATTTTTTATTTTCCCTTGC
CTTTTCTGTGTCCTGTGCTATGAGTAACGTCGTTGAATTATTCTTCCAGCGCACTAATTTTTCCTTCATTTGTGTCCAATCTGCTCTTACGCTC
ATTCACTGATTTTTTTTAATTACATTTTTTAGAAAATTTCTAGCAGTTCTGTTTGTTTGTTTGTTTGTTTGTTTTTTCAAATATTTCTGGGGCA
TTTTTAGAGTATCTTATTGGTTGCTAATTTCCATGGTTCCGTTTTTTATTTTGTTAAACATTTCATAGTTTATATTCTATATCTGATTATTCTA
CTACCGAAACTTTTGGAGGATGTAAATCTCTCTCTCTTTCCCTCCCTACCCCTTTTCTCATAACTCATGCTTCCTTATGTTTTCAGTCATCTTT
CTGAGCTTATGCCTGATTGATATTAATCTGTGGGAATCCTGAGTATTCGATGCTGGGAACACTTTCCTCCAGAGTTGAAGGGGATTTCCTCTTC
ACAGGTCCTTGCTTTAAGGTGGGAATCCCACATCCAGCTGCTCACTGGGCTGCTGGCCTGAGATTCTGTCCCTGTCCACAGCTGTGCTGATATT
GGCATTGGCCCTTTGGCCAAACCCACTTTAATTGCTCTCATTTGCCACTCACTTTTTGCTGTTCAAGTTTCAACTCACTTTTTGTCCTATACTT
CAATTTCAAAAGTGAATTTGCGGCTTAGGAAAAACCTTTTCCTTGTTCATTGAGGAATGAGACTGTGTTAGGCTGTCACGAGCCTCCTTTGCCA
CAACAAAAACTCTGTTCCATCAGTGCTTTCATATCCTGCCGTAATTGATCAAAGTATGCCATTTTATTTGACACATGTGGTTTTGCAGACCTAG
GTCATCATAAAGAGTCATGACCTGTCTGAAGTTAAAACTTCAAAGAGAGTTGTGGGCTGATGTCAATATCACTTCATTGATGCTGATCAAAGTC
TCCTTGAAGCTGAAAGACAAGATGCGTTAAATAATTCCACCTTCATGAACTAAAGCAGAGGGAGAGAATGAGAGGGCGTTTGTAATAAAATGTT
TTAAACCACAGGTGTGTTAAAACAGCTTGGTGTCGCCTCAGATAGGGATTGTGTACTGATCTTGCTCTTCTTTCTGTGGAATGCACATGTGCCT
CAGTAGTGCTATGGCGCAAGGCAGAAGCACTCAGAAGTTGCATTATCTATATCTTTCATGGATCAGTAAAGCTGGCATTTCTCCTGCCTCTTGC
TGACATACACACTGTTCAGGACCTCATTCCCTCCCACATCCTCCTCTTCCCCTCTCCCAGCTCTCTTTGAGAAACCACTGTTGGGAGATGGCTC
TGAGTCAGAACTGCCCACCCAACCCAGCACAGGCCCCCTCTGTCCATCTGCCCAGCCCACCTTAGCCACAGTGAGCATTCATCCTTTGCACATC
TAATGCACACAGCCAATCCCAGGCATCTTCCTTCCCCTTGTATGAGTCTGACAACTGACCCGGTGATACTCTACTCTGCCATGGAAAAAAAATG
GCAGGGGAGAATGAGAACACCTAGTGGGCTGGAATGTGCCCGGCTTTTCTGTTCCGTCTACAGCAGATACAGGCAGTGTCCCCGTAGCCCTGGG
GAGGCCACCTTGAGTGCCTCACTACTTCTTACATCCTCACTTTCCCCTTGGTGCCCGGGCATCACCTCTGTATATTCTCTCTCCCCTAGGAGGG
TGCAGGAGAGTAGAGGAGTCCAGCCCTTCCAGGCCATGCCCCAGCAGAGTCAACATCCCTCACCTTTGGATAGTGTTCTAGTGTCTGTGAGGCA
CAAGGCCTGCTCACCCCAGAGGCCAAGGCTGCCTACCCTGGGAAAGTAGACATGCCGCAGGTCAAGGACTGTCTCAAGACCAGTTAGGACAGCT
TCTCAGCATCATCTCAAGCTCAAGCCCTGGAAATCTCAGGCTTTGCAATAGCAAAACACAAATCTGTTAACTTACTCCTCCCAAGACAATTACT
GGGAAAAAATATTTTGTAAATATTCAAAGGCTTCTGTGAACAGCCTGAGGCCAGATGACCCTGAAGGCGGAAGGCAGCAAGCCAGACAGGAGCC
AGGAGCCTTGCCTGTGTGCACGCCCTCTCTGGGTGTCTGTAGAGCCATAAGCCTGGGTGGGTGTCCTGATGCCACCACCAGGGCAAAGCTCTCA
GTAAGAGGACACAGCCAAGAGGAGGAGCAAGCCTTCCAAATCAGTTTGTCTCCTATGTTTAATTAAAAATAGCCTGGTCCAGGATCTGCCTGCA
GCCAACAGATACCTCACAGTTCCTGGTGGTTAATGTCGCGAGCCTTCTGGTCTTTGCCACCTACAGATGCTGGGCCTGTGGGTTTCCTTTCTCT
GCTCTTGTTTCCTGCCACCTGCCACAGGGGGCTGCCCAGGCCACATGTCCATGGTCACTACCTATACCTTCCAGTTACCTGTTGCCAGGACTGG
CCAGGGCTGCTTGGTGGAGCACAGGAAGAGAGGGGATCGCCTGCAGAACCTGTCCTCTTGAAGAGCCTCAAGGAAGTGAGGCCTTTGCAGAAAC
TGCTTGTTCCAAAGCAAAGGGCACTCTCTGTTTCCTCTGGGTTGCTCTCTATTTCCTCCTGGTACCAGCTCTCTTTTTATACTGCTTTTATTGT
GAAATATAAAACAAACACATAAGAGTGCATAACTAAATAGGTACAACTTAATGAATGATTATCAAGCAAACACAACCCAGTCAAGAAATGAAAT
GCACCCAGCATTGCAGGAGACCACTCCGCCCTCACACACTCATACCCATCTGTACAGCTGGCCTCCTGGGAGGCACCACCTCCTGAACTCTTGT
GTTCATCACGCCCTTGCTTTTTTTGTTATTTTTCATTTGTTTGTTTTTTTAGTTTTACCACCTAGGTGTACATCACTAGATAAAACAGGTGGCT
TTTGTCTGTTTGGGGACTTTGTGTATATGGTAGCATGGAGTGAGTTTTATGTTTGTGAGACTCATCTGTGCTTTGTATAGCTACCATTTGCTCA
TTTTATTGCTTTAGAATACACTATTGTGTGAATTCCTACAATTTACTTGCTCTGTCGTCACTTACGTTGTGTGCAGTTTTTGATAAGCACAGAT
GAAGCTGCTATAAGTAGCCTTGGGGTGCATAGTGCACACATTTCATACACCTAGGGGTGGAATTGCTCATTCACGAGGTCTGTGAATGAATAAG
TTCAGTTTCAGGAGACTGTTCCAAATTGTTTTCCAAAGTGGGTGAGCCCATTTATGCTCCCACAAGCAACGTAGGAGGGGCCTTGCTGCTCCCC
ACTCTCAGTAGCACTTAGAATGATCTTTCTAACTTCCACCAGTCTGAAGAGCGTGGAATGGTACTTCCCTGGGGTCTTCATTCATGTTTGCCAG
ATGCCTAATGAGGTTGATGGCATCATCTCTCAGGGAAACCTGTGCTTGTCTGTCTCAGTTTTTAACAAGAAAATGTCAATCAAGACATAGGGTA
AGATGGAGTACAGCAGATTGGACATGGATTCAGAAACCAGAGTTCAGATCCCATTTATTACTACTATCTTTTTTTCTGGAGAGATGGCGTCTCA
CTCTGTTGCCCAGGCTGGAGTGCCATGGTGCAGTCTTGGCTCACTGCAGCTTTCACCTCCTGGGCTCAAGTAATCCTCCTGCCTTGGCCTCCCA
AAGTGCTAGGATTACAGATGTGTGCCACTGCACCTGGTTCAGATCCCATGTATTATCTCCATGACCTTGAGGTCACAGACGTAAAAGATTAAAT
TATAGGAAATACTTTCGAGTGACATATTGTCACCATTCTTTCAAGGCCAACTTTTATAGGCATATGCATCCACTCTTACTGCTGTAGTATTTGG
ATAGCTCTCTATACCCTCAAACTATAAGCCACTGATATGTATAGAATAAACATAAATAGTCTCACTTTATAAGATACTTTCCCTATAAAACAGA
CTCAGTTCTCTCTTTATATGAAAAGATTACCATGTGGTTTTTTGTTGTGTTGTGTTGTGTTGTATTTAACAGTCACCAACTGCTGGCTGTAGTC
GGCCATGTGGCTGTGAAAAGGGAGCTCACACCTTGGGCGCTGGCAACTCGGTTGGTTTGGGAGGAAGAGGTTGTTCTCACAGTGGGGAGGTTTC
AGCACAGCCACCTGCTGAGGCCACAGAGAACCTAGATATGTGTCCTGTACCCTGGCCAGCTGCCCTGAGCTGTGGGGTGCTGTGCATGGCCAAG
CACCGCTAGAAAAGACACTTATTCTGAATTTCACTCCTCCCATGCCATCGTTCAGCTTTCAAGTCTTGGGTTAGCTTTTTGAGATAAGTCTCCA
TGGCAAGGGACAGGAAAGCATGGCCATGTCCAGTTCTGAAGGCCATCTCAACTCTCCCTGTGGTCGTGTATCTGACTTCTTGCAACCAGGAGGA
GGCATCAGAAATTTCCCCAGTACCCAGACGTCCCAAGGCAGTTGGGCACACTTGAACTCTGCCCCAAGTACAGTGCATTTGGGCACGTGGCTTA
ACAATTTTGGACCTCAGCTTCCTCATTTTAGAAAATGCAGGGGCCAAAGAGGCTTCCTGGGACTTCATGACTCACCCTCAGAACCTAAGATCTC
ATGAACCAAAGGGCTGCAGCTGTGGAGAGGGGAGAATGGGGCAGTGTGTGTGACTACGAAGCTAAACTGGGGGATTCGCACCCATTTCGTGCCT
TTTCAGCCTCCACTGGGACCTCGAACTCCCCAAGTCCCACAGGTCTTCCAGGGGATCTCTCACCTCCCCATGAGTCATAAGCCTTACGTGGCAT
GCACTGCAGTGGGGGTGTTGTTCCCTGCCTGGCATCAGACTCCTGTGTTTCTGTTCACCAGGACCCAGGGCAGGGGGTTGTTCTGAGGGTCGCC
CAGCATTACCATGAAGACAGAGCCTTGGCTGCTCAGCACGGCCAGCACAGCTCTGGTTTCACCTCTGTCTCTTCACCCCACAGTGGATGTCATT
TTCTTCTCCTGGGGGTGTTACCCGACAATATCATCATTTGAAACTGTTTGAAGTCCAGCCCTGCCTCCCTTTCCTGCATTGGGAGCTTGGAAGT
CCAGCCCTGCCTCCCTTTCCTGCAGTGGGAGAGTGGAAGTCCAGCCCTGCCTCCCTTTCCTGCAGTGGGAGCATGGGAGGGACTGAGGAAGGCC
CGGGGGATGGAGGCTTCCATAACCCTCCATCTCTGGTCCAGAAGATGGCTGGTTAAAATGCCCACCAAACACCCCCACCCACACTCCAAGCTAG
GTGGTAGAGCTGTCCATCTTGGATCTAACCCAAATGCAGGCCCTGGTGAAGAAAGGTGAGTCCACAGTGCTCCACAGATAATCAGGGCCCGTGG
TGCCCCTGTGCTCATCACCCTGAGAAGCGTCACTGCTACTGAGCACCAGGCTCTCCTGACCACTGCCCAGAGCTCCTGGGCCCTGCTAGAGCTT
AGCCATGGTCCATTCATCACCAACCTGCTACCATTTCCTTGTTGCAAGCTTGCCCACTGGCATCATCCCTGAGTGGTAAACTGGGCCTTCCTGG
GTTGGGTAAAGCCCTTGACCCTGAGGCTCCAAACTCTGCCCTCCAGCTGCCTTGCCAGGTGACAGGCCCCAGAGAGCGGCTGCTGACCATTCAG
GCCACGGTGCTTCCTCCCAGGGTCCCACCTGGAGACTGGCGGGCCTGGGGATGATGCAGTTCCATCCTCTCTTCTAGGATCCGAGCCAGAGACA
ACAACGGCTCCTACGCCCTGTGCCTGCTGCACGAAGGGAAGGTGCTGCACTATCGCATCGACAAAGACAAGACAGGGAAGCTCTCCATCCCCGA
GGGAAAGAAGTTCGACACGCTCTGGCAGGTACCCAGCCTCCTCTCCCCACCTTGTGGGTAGAGTACAGGGCACGTGGGGCTCCTTGTCCCATGG
ACTCTTAGACATGAGAGGAACTGGACCACCCACTGCCCACAGTGTGGTCCCCAGGCCAGCAGCAGCTCTGGAGCTCATGAGAAACACACACCTG
GACCACACCTGGGCTCCTGCCCCACCTGCCCATCAGACCCCACTTTCAGCCTCCAGGTGGCTTCTGTGTTTGAGAAGCACTGGTCCAGAGGCCG
TCATGTGAGGTGAACGAAGGTTCCATGGCTTGTTCAGGGCCACTCAGCCCTCAGTGCCACAAGGTGATGTAAGTGCAGGGCTGTGTTCAGAGTT
TAACAAGACACTCTCTTTGGAGATCTAGAAAATATTTCTTCCTAGGATATTCTAACTAGAGCCCAGAACAGCCTTTCAGACAAAGACACACAAA
ATAAAGGAATGGATGCCTGGGGAAGGAGGCCTAGGAACAGCCAAGCAGGACTTTGTTGGTGGTGTGAGAGGCTGAATTAGACAGCAAATGTCAG
GTGCCCTCCTCATTGTAAATCGAGTCTCTCTCTGTGGAAACTAAGGCAATTTAGGAAGCATACTCCCTGGGCTCCAGGCTGCTGCTTATGCTGA
TTGCCACAGCCCTGGTCTAAAAAGTGCTTATTCCTGACCCAGCTACTAGAAAACCCTGAAACAACTCCCAATTTGGAAGTTCCTTAGGCACTAA
TGAGCACTAAAATAATGCTCATCACTGCTTTCCCTGAATCGCAGTGAGCACAGATTGCTTGTCTTAATTTTGCATCACAGCTACACTGGCTGAT
GATGAAGGAAATAACCGTAACAGTACTTCGTGCATCATCTTCGCAGAGCATCCTATAAACTGTAGTAGGTTCAGAAACAGGGAGAAAGCCAACC
GACTCTGCCACCACCCCACCGCCCCAGAGCACCAGCTACCTTGAACATGTGTGGGTGAGGGGCATAATCAGGGCGTGTTCCAATGTCCTCACCC
AAGCCCAGCTGGGGCCACACATGCAGTCAATGTTGACGAAAGGACTCAACTCAAAATCCTGTCTTTTCAAAAACTGAGACTACAGAGCCTTAGC
CCCTTTAAAGCACTTAAACTTACTACTTCCAAATGTGATTTGCTTTCTAGTGCTGTAATTGGAAAAAAAAAATACTCCTTTTTTGAATGCTGGA
TATGATCATATTAAAAAATGTTAGTCACAACATTTACATTGTCTTTTGATTTTGTGGCTCATTGCCATTTGTCATCATTAACCACAGGCTTCTT
TCTTTTAACGATATTGATTCCAAAGAGTATTTAACCTTTCTACACTCAAAGCCCCAAAGCACATCAGGGAGGTGAGTAGGGAATGTGGCCTTCT
GGAATGCAAAGGCACCTGGAGTCCTCTATGCCAGCTTAATGCTCCAAGGTCCCAGGCCCCATCACTTATAAACAGCCATTCATTCACCTTCTCC
TGAAAGGGCATTTTATGTGTGTGGCGTTCTGTCACCATTTCAAAGGCTGAGCACTCACCATAGTGTTAAGCATCTCTAATTATTGAGAATGTCC
TTCCCAAGAATCTTTACTATTTGTATTGATTCCCCTGGTAGATCATAGCATTCATCCCTAATTAAATATTCCTCATTGTATATTTTCTAGGCAC
TGTGTTTAGGAAACTACGTCAAAAATGCCAGCAATACGTTCCAATTTCCCATGCATGATTTCATTGATTTTTCTTGACAGCGTTCTAAGGTACT
TCTGTACCCTACCCAACTCCAGGGCCCCAGTGCTATTTCACGGAGAGTTGGTGATTGCCATAATACCCTATTTCAATATTCTTTACTGTAGACC
AGGGATCTCCAAACCCACAGACCAACTCTGGCTCTCAGCCTGTTTCTGTGTAGTCCCTGAAGAGAATGGTTTGTATGCTGTTAAAGGTGTTGTT
TTGGATAAATAAATAAATAAACAAAGGACACAGATACGGTATGAGGCCCACAAAAGCCTAAAATACCACTTTCTGGCCCTTTACAGAAAAAATT
TGCTGCCCCTAGCCCAAGAGGGGGGATCCCTAGCTGTGGCCCCACTTCCCCAGCCACCCTTGTGGAGTGGACTTAAGCACATAAAAGCATTTAT
GTGTCTTGGGCCCACTTCGAAAGCCCAACAGTCAGTCAGTAGCTCTCAGTCACTATTTGTCTATTTCCTGACCTTGGTATTTACCACTTTCTTA
CTTTTTTCTCTTTCAGCTAGTCGAGCATTATTCTTATAAAGCAGATGGTTTGTTAAGAGTTCTTACTGTCCCATGTCAAAAAATCGGCACACAG
GGTGAGTTCCCAAGACACTGGAGTCTCAGTGTTTGAGGTATCAGTGACAATCAGCCTCATTTTAGACTTAGCTGATTGCAGATTTGCGTTGGAT
GAGGACTTCTTTTTACGGGTTGATTAATAATGATATCAGAGCACTCCGATTTTGTATATCGTGAATCAAAAAATAGCCAGTTGCTTTAACTTTA
AAAAAATTGTAATTTATTTGTCTTCATTGCATGCGGGAAAAAGTAAAAGAGGGTCGAAAGAAGTACCTGCAGAAAGCGTGCTCACTTCTCAAGC
AGCAGCACATCCTGATCTCATTGATTGATCTGCAGTGGGGAGGGGAAGGAAGTGGTTTCCTCAGATAGAGCAGTAATTGTCCATGCTCTCTCTA
ACCAGGAAATGTTAATTTTGGAGGCCGTCCACAACTTCCAGGTTCCCATCCTGCGGTAAGTGTCACTAGGAATACCACTGAATGAGAAGCTGTT
GCATATTTCACAAATGAAAAGCATGTTTTAGCTAACAGGAGTAGTACGCATTGATATTTTGATTGCGCTTCAAAACATACTCCGTGATGAGCTG
GGATTGCTAGTGAGTGTTAGGCAGAATCAGCCTCCTATTAGTGATAAACTAACCACATTTTCTTTTCTTTTTTTCTTTTGCTTTTTTTTTTTGG
AGATGGAGTGTCTCACTCTGTCACCCTGACTGGAGTGCACTGGTGCAATCACGGCTCACTGCAACCTCTGCCTCCCAGCTTCAAGCCATTCTCA
TGCCTCAGCCTCCCSAGTAGCTGGGATTACAGGTGCCTGCCATCACACCCGGCTAATTTTTGTATTTTTTAGTACAGACGAGGTTTTGCCATTT
TGGTCAGGCTGGTCTCAAATTCCTGGCCTCAAGTTATCCTCCTGCTTCGACCTCCCAAAGTGCTTGGATTACAGGCATGAGCCACCACACTCTG
CCTAGCCACATTTTCTTAACTTCTTGTCTAGATGAAATTCTCTTCATTTTTGGAGAATATACACAATGCGAATGAAACCCAATATTAAATCAGA
AGTAGGCCTCTTGCAATATTTGAGAAATGGGAACATATTTTTTAGCTTCATCCTTGAGCCGTCTATGTTAACACACGTAATTCACAGGACCTCA
GCTGTCTCCTCCCTCCAGCCTGTTAGGAGGTCAGGTACAGATTTATGCTCTAAGCAGGAATTATTTTCCTGCTCTTTTCTTGGGGCTACATATG
GCCTTTTTTTTTTTTTTTTTTTTTTTTTTTTGAGACGGAGTCTCGCTCTGTTGCCCAGGCTGGAGTGCAGTGGCGCGATCTCAGCTCACTGCAA
GCTCCGCCTCCCGGGTTCACGCCATTCTCCTGCCTCAGCCTCGCGAGTAGCTGGGACTACAGGCGCCCGCCACAACGCCCGGCTAATTTTTTTA
TATTTTTAGTAGAGATGGGGTTTCACCCCGTTACCCAGGATGGTCTCGATCTCCTAACCTCGTGATCTGCCCGCCTCGGCCTCCCAAAGTGCTG
GGATTACAGGCGTCAGCCACCGTGCCCGGCCCATATGGACTTTTTATGCCCTTTAATGCCATCTGTGAAGTGCGCCCTCTGCACCTCACTTTTT
GCCTGGTAACCCTTGAAAAACAAAATCTGCAGAAAGCCAAATATACATCCTGCCTCCAGTAGGGACAATTGGCTGCCCAGTAGCTGAGCCCAGG
TTGAAACAGGGGAGGCTGTCCAGGGGCAGCACTTGTTATCAAATGCAGGGAACGTGAATTTGCTAAAATACCAGTGACCTTTGGGATCAAAATG
TAATGCCTTTATGGAAGTGTGACTTTATTTTGCCCTTTACAGGGCTCACAGGCCTGGCAACTCTATCTTAATATTATAGGCTCGGTGAGACAGA
TCCATACACCCTTCAGATGGATGAGGGCTTAGGGCTGAGAACTCTTAAAAGAATGTCAAGCCCTGTGCTTAGCCATCATCACCCAATGACTACA
AGAAGTTAGATATTTTGGAATAACTTTTTAATTGACTGGTAAGATACATACGGGAAAAAAAAGGCATACGCCCTATGTGTACAACTCAATTAAT
TTCCACAAAATTTTCCACCGTGGAACCAGCACCCACCATGAAGAAGCAACACAACTGAGCACTCAGATCTACTTGTAGTTTCTTTCGGTGGTTT
GTCTTGAGCTTACAACTTAATAATTCCACGTGAAGCATGAGCTAGTGAATGTAACAGCTCTAATTTTGTAGCCTTCAGTGGAACATATTTCCCA
AATATATTTCTAGATCCTTCTTCCCAAGTGGCAGGCCCTTCCAGAAGCCTTCCCAATGATCAGGTGTTGCCCGTGGTCGATCTCATTGGCAGGG
ACAGGAGGGGGTTAGTGGTGCGATTATAGAAGCAAATTTAAGTAGCATGTCATGGAAGGGATCCTGTATTTGTTTTCTGAAACATTTACTGTTC
CTCTTTGCCGTTGTGGTTTCTAGACTTGGTCAGCGGGTGGAATAATCTCAAGAATCAAATCATACTCCTTCCCAAAGCCTGGCCACAGAAAGGT
GCTAAAGCAACCCCTGCTTGCTGTCCAACTAAGTGGTAGGACCAACGCGCACTCAGCTCCCGCCCGCCCAGTCTGCCCTGTGTGTGCGCTGCCC
CCCATCTCTTGCCACTCTGCTTCCAGGCCTCTTTGCCCTTGCTCACACCTGCTGTTCCTGGGCAGCCGCGCCCCAGGTACATCATGTGCTTTTC
ATGTGGGTCCTCAGTCCGTCAGCTGGAAGTCCAACAGTGTGAGCATGCTTTCTGTGCTCAAGCGTCATTTTAAAGTCGTTTCAGTTGGCGTCAT
CCGTCATAGCTAATTATCATGTCGGAAAATCTTTTCAAAGATGGCCCACTTACTTTTTGGTGCCCAGACTCACGTGTTGCAGTGTTCTGGTAAT
TGATTATGTAAGTTACGTTGTGATGGAGGCAACACCAAGGTTTTGTGAGGCGCGCTGCACAGTGAGGTCAGGCAGTGCATGGTAACTTCATTAT
GGCTTAGACGTCTAACATACAACTGCAAATGTGACATGTAAAGAAGAGGTGTAATCTGAGGTCTAGAAAAACCAGTTTTCTTTTTGAGGCAGTA
ATGTTGACATCTCATTGATTTCCATCTTTTGTTCTATTATTGGATTTTGAGGGAAAATAAAAATAAGTTCTTTTTCATGACTTTTATTTATAAT
GACTTATTTATTCCTTCCCCCAAAATATGTATATATAATGTGTTTGAATTGCTTTTTTTCCATTCTACTTTTATTATCAGTGGGGTAAAATTGG
TTCTTTTATCAGTAATTCTTTTTATTTTCATATTACTTGGTGTTTTTACCGACATTTTAGCATATAATTGAGCATCCACTCATGAAATAGATTG
CCTAAAGTATTTACTAGAAACAACAGAAATAAAAGACATAAAACCCTTGAGATTTTATTTAGTAATAATTTTCCAAGTTTACTCATACTCAAGA
AGTTCTCAATCGTGTTGACCATAGAACAGAAAGATTTAATGCAGTGTTCGAGAGGAAGAAAAGGTTTATTTTACACTTGAATAAAGTAACTATG
TCTGTTTTTCATGTTTACCCAAGGCAAAACACCCCCACAGATTACTCACAGTGGCTAAAGAAATACCTAGAAAATAAGTTTTGGTGCATCTTTA
GACTACAGGAGAACATGTCAGAAAAAAGTTGAAGAAAATATCAGTGACCACCTAAAAATAAAGAAGTATATGTGTAGATAACAACCATAACATC
ATCAAAAGAGAAATGAGAAACTGGGAGTAAGTAGCACTAATGCTGACACATAGATTAATAGCTTTAGTATACAAAGAGATTCTAAAAATAATTA
ACAAAAATATTAACTGTCTACTAGGATAATTGGCAGGGAATATCAGTAGTAGTTTATAGTAAAGCAAATATAAATAGTTCTTAAACATATGAAA
TGTTGTTTAACTTCATGAGAAGAGAAATGCAAGTTAAAACTATGGTGCTTAACTGTGTCCACCTATGATCCAAATTTTAAAAGCTTGATAACAC
TGTAGTAAAGGCATGGTAGATAAGCACTTTCACTCTTCCTTGGGGAAGTCTGAAATGATGCAATCTGTAAGAAAGGCTCTGCAATTTTATTTCT
AAGTATGTACAGATATTCTTGCATTTGTGTGAAATCATGTACATGTGAAGGTGTTTTTTCCAAAATTGTATAATTGACAAAGTCTGATAATAAT
TTAAATGCCTATCAATGGAAAACAAATGAAATTGGTTACAGTAAAATTGCTACTATGGGAGGCAGCTGAGATCTAGAATTGTTTTCAAAATATA
ATTTTAAGTGTAAAAGTCAATGTCCTGCAACACACACATACACATACATATATGCAGAATGTGTTGATATCGTATACAGTATCCTAGAAAAATA
CAGAAAATGCTGGCAATGCTGGTTGCCTCTGGAGTAAGAGATGGGAATGTTAAGAAATCCCACTTTTCATTGTAAACTCTGTTGTATCTTTTTA
AATTTGAACCTGTTAAGAAATTTTAATCAATTTTTTTAAAACAGAAAATTAAATAGTAGAGAGTAATTAATCTGATTAATATTGTGAAAATACC
TTTCTGGCAAAAAAAAAAGTTTGCTTTCTATTTAAGTTTAGAAGAACTTTAAAGTATACATTGAATTTTTTATAGAAATAAGAAACTGGAAATA
AATATTACTAATGATGACACATATATAAGGCCAAAAATATCAACAGATAATTTTTAAAAGATCAAGGTTGTAAGGTTTAAGCTATAGATTAAAA
ATATTTTAATTGCTTATCAGAAATATTTTAGAGCTAATATGTATATTTTCACAAACAAATGGGGAACTTTCCATTATGTAATCAGTCATGAGAA
AAGAATTGTGAGAATTATGTCAAATGTCCCATCATGTTACCTAAGAGATAAATAACTTTCTTGAACTTGTTAATTTGCTCAGTGTACATAGTTG
TTTTAATAATTCAGGCACAAAAAATACATGTTAGAACTTGAACTACCTATAGCATCTAGATAGTTAATGAAAAGTATGTTTAGATTTGAAGATG
ATTATGAATCAGATAATTTAAATTTTTCATAAAATTACTAACAGGCTGATGCATTTTAAACTTGTCTTTAGAAATTAGTAGCTTACTGCCAAGC
ACAGTGGCTCATGCCTATAATCCCAGCACTTTGGGAGGCCGAGGCAGGTGGATCACTTGAGGTCAGGAGTTCAAGACTAGCCTGGCCAATATGG
TGAAAACTCATCTCTATTAAAAATACAAAAATTAGCCAGCCGTGGTGGCACACGCCTATAATCCCAGCTACTCAGGAGGCTGAAGCAGGAAAAT
CACTTAAGCCTGGGAGGTGGAGATTGCAGAGATCGTGCCATTGCCCTCCAGCCTGGCAGACAGAGCAAGACTCTGTCTCAAAAACAAAAAAAGA
AAAGAAAAGAAAAGAAATTAGTAACATACTTATAAAGTATAAACAAAATTAACTTAAGAATACAACTAGTAGTCTTTAAATGAATTAAACCCCT
CCCAATATTAGTCCTGGAATTTCTTTCTCTGCTGCTTTAAGCACAGAACCTTATCCTTCTTTTATGGAAAATAGGTGGAAATTAAACAGCAACA
TCTCTAAAAAGACTTCCAGGGAACTTTTCAGCTTTGCATTTCAAAGGCACATCCCCTAAGAGGATTAAACTGAAATGTGTCCCAGAATTTCCAA
TTAAAACCCATTTTTCATGTTTTATTCCAACACATACATTGGTGATTATAATATTTTAAAACTTACATTTAAGTACAGGGCTCAGTGGGAAGAG
AAAAAAGAAAGAAACATCCTCAAGTTGTACCCTGTTGCTTGGTAATGCCTTAGTAGACCAGACCCTTGAGAAAATGTGTTTTCATATAATTATA
AAGGAACAGATTTTAGATCTTCACTGTAAGAGAACAGTAATTGCAGAGGTCAAGTGTTATTTTCATGGATCTCAGAGCAAAGCTAATAGTAGCC
TATTCCCCAGCCAACAGTCTGCCCTCGCCTGTGGGGGTCTACTGAACCAGGGTAATTCCACCAAAAAAAGGAAACCAAACCTAGTTTATTGCAG
CCAATATTTCTAAAGCCAGCTGGCTCTTTTGTCACTATTATTGTGATGACTAATGACTGTATTGTCATCTGCATTACTGCTGTTGCCTAATTTG
AAAATGGCCCAAGAATCTGGGAGTAAAAATTAAGATCTAGTAAATGGATGTTAGGACACTTGGCAATACTCTGGAATCAGGAAACTCTGAATGG
ACCTATCAGTTCCACAGACCAATAATTTTTTCCTCTTTTGTTCACTTTTGGATCACCAGCTTTTAAACATAGGCACTCAAAGAATGCTTGTCAA
ATACATTTTCTATTCGCAGTTAAACCTTACCAATAGGAAATCAGGGATAATAATTTATAACTGGACTTATTATTGCAGAGTGATATCCTTACCT
GAATTTATAAAATTTAAAGAAAAGTCACCAGTTGTTCTTTTGGAAGGTAGAAAGCGAAGGTGGCTAATGCCATCTAGCGGTGGACTGAAGGTTT
GAGCCTGTTCTGAGTATTAGCAAAAATGAAGAAAGTGGAATGGTCAACATTTCATTTACACCTTGTTGCTCACTGTGCAGAACAGATGACACTT
ATTGATTGGGACAGAATGGCTCTCTCAAATGCAAACATAAAGAATAAAAGGTTAATAGTAATGTAGACCGGAAATATATTTTTTATTAAAATTA
AATGTCATTACATTTTTTAAGAATAGTATTTTTACAATTTATCATTATGCCACTCTGCTCATTCTTTACAAACTCCTCAAATTTTTCTTTTTAA
CTGAGCATTTTCTTTTAGAGATTGTTTTGGTTATTGTTCTACTAAGTGGCATATTCACCAAGTGCTTTGGGAAAGATGAAGCATGGATTTTGCA
TCTTCATCTTTATCATAAAGTAACTACTGTTCTTTTAACTGACTCAATAGGAAACCTCCCATTTCTTCCACATTCTTCCAGTTGTGGCATCATT
CTTTGCTCCAGTCCTGTTAGTCACCAGAAAGAGATAGGGGCAGGGAGCCCAGGGATCTGGGTAGGAGAGAGTTGATCTGGGGTTGTTGAGAGAC
ATGAGGGTTCTTTGTGGCTTGGCTTAAAAGGCTTATAATCACATAGTGGTCTGTGTTGGGCAGCATGGAAATGAAGCCCCAAGCTTTATTGGCT
ATAAACGTGGAGTTCTAATTTCCGAGGAAGATCTCCCATCTGTTTCCCTATCCATAAGCCAAGATAGACTAGGGGTGAACAACACACTGCTAGT
GAGAGTATAAATTATTTGTGCCCAGGGGTTTAGATTCTCTCTGTCCATCCCTACTAATTCTTACTCATTCCTGACACCCTAGTATCCCTTTCAG
CATATTTTGACAACCCCATCTCTGTTTTAAAATTCTTCCCTGCAGGATTCATATGTCAGGGAATTGCGTTCTGGTTTAATTGGAACAAGCCATT
TGGAAGTCCTCCTGGTCTGGAGAACATTCTGTGAGGTAGAGAGGCTGCAACCCCAGGATAATGTCCTCAGTGGGAGGGACAGGAGGTCTTATGT
CAAGAAGAGGTGATGGTCCCTCTCCTCACCAGTGATGGCTAGTCCGAGGACATCCTTTAATTCCACATCTGTTCAAGTTTTGTCTTTGGATCTC
AGAATGTGCCTGCCTGCAGGGAAAATCAAAGGAGTTGAAATGACAGTAAATATTAACCTTGAAAGAGACTTTAAAAAAAATAAAACTATTTTCA
GCCTAGAATTGGTTGCGGGAGAAAACTGGGCAAAAGAGGAGGACAGAAAGCCTAGGGGATCACCCAGTTTCTGTCAGGACAGTTTTCCAGAGGC
AGGATAATTATATTATAAATCAGAAGTGAAACCACAGCACAGTCCTGTAATCATATGTAAATGTTTAAAAGAAATAGATTGTCATTTGGAATTA
TTCATTCTTATTTCTCTTCATCAGCATAAAGGCTTGAGAGAGAGCTAATTTTTAAAGCACATTAGATTATACTTCACATCTCCTAGTTTTTAGA
AGTGATTTTCATTCATTCACCCATCCATTCATTCATGTGTTTGTTCAACACATTTTTATTGAGCGTCCTTGTGGACGTGGCACAGTTCCGAGTG
CAGTGAATAAATGTCTCCCCCTCCTTGAGCTTATATTCTATTTTTATTTTTGATGTGTCATAGTTACTTCTGTTAATCAGTTCAGTAGAAGTGC
TGAGATACTCTGTGACAAAACAGATCCCATCGACATCATTTTTACAAATAAGACCAGATGTCCTTTAGCCTTGTCAACAGGCTTGGGCCATGGT
GAACAATGTCTGCCTCTGAAGTGCTCAGCAGACCGTGTGAAAGAAGAAGGTCCGGGTGTTTGGAAACAATGGCTGAGTGGCGGTAGTGGTGCAT
TGTGACCTCATTATTCTGCTTTTGGTTTCTCGCCTTGTAATATGAATAAGGCACATGAGGATTTAGTGTGTGTCATAAGATAAGTGACTGTCAC
GTTAAGCTTAATGTCTCTGTTTCTGTAACTTTCTCCATAATCTGGCAGTATCTCATTTCTTCGTGGCCAGTGTTTGGGCACTGAAAAGGTCAAT
CAATACATAGAATTGTGACTCAACTCACGCTGAAAAAAAAAAAAAAGCATCTGAATCATGGTTCTCCTGTGAATTAAATTTAGTAGCAAGAATC
TTGTTTTAATTTGGAAGAAAGGGTGGGTCAGGAAAACTCAAGATAGAGACTTTCAAAAGGGAAAGTCCTTAGGATAATGAGTAGATTTCCCCCC
CCAGTTGTTTCATGATATAAACTGATGTGGACCAAATAAATACCTGTCTCACAGGGGTACTGATTTCCAGCATTAGCTGTGGCAGCTGGAGCAT
TGCTGGCCTAGGTGGCAGCAACTCGCCTTCCCTTGGGACCTGTAGTTACTGACCAGTGCCACCCTCTGCCATCCACCCCAATGCTGCTGCATCT
CCACCGAAGATAAAGACTTGGGTGGAGACTTGGAAAACATATGTGCGTTGGAGTTGGGAAGTAGGTAGGAGCAGCTGATGAGTACTAATTTTCC
TCTTTAAATAATGTCCATGTAATATGAAATTGAAGAGCACAAGTTAAAGAAGGAAAATGCTTCCAAGGCGAACATGCAAATCATCAGCGATCTT
TGGTAGGTGGGAATAGGATGTGCAAGGGGGTGAGTGGGGAAGCAGCTCTATCCAGGCCAGTAGAGTTGACTCTCACCACACCCTCTGAACACCT
CACAGAGTCCCTTGAGTTGTAGCAGCACAAGCTTGCTTTAGCCCTTGCAAGATCCAAATAATTGCCCGAAGAATGAGATCAGCCTTATTTTCTT
CCCTGTTTTTCGTAATTCTTTCCTTTGTCTTTCCTGCAAAAGTTAAACTGTGATTATTGGAAAATAAAAATAATTATGCAGATCAACTTAACAG
TTTTGTGGATGAAGAAAAACAACCTGTTGTCCTTTATGTACTTTAGTCCTCCCCTGCCCAAGGGAACCGGCAAGAGAGTACTGTGTCATTCAAT
CCGTATGAGCCAGAACTTGCACCCTGGGCTGCAGACAAAGGTGAGACTTCCTTTCATTCAAGGGACATTCACAGAGCAAAGTGCGTGGTCACTT
GTAAGTTTGTAGAGTTGGTTCACGAATCCACACCACGTCCGTGATTGCAATACAGCCACAGTTAGCCATGGAAACACTCACATCTAGTGGCAGG
CAGCAGGCACCACATGCCCATGAGCAGTGATGGTGCTATTGAATGAAATGGTCTTTCCCACCTCCCTCACCCAACGACGTTGGAAATGTCCCTG
CCACTCTTCAGAATTTCTCATCCCTTGCAACATTGATGCTACTCTGAGTTCATATTCCCATGAAGATCATGTTCTTGGAAGGATGAATCCTGGT
GTGGGGTCCAGCCCCAGGTCGTATGTTTCTTGACTGCATTGCAGGCCCCCAGAGAGAAGCCCTACCCATGGACACAGAGGTGTACGAGAGCCCC
TACGCGGACCCCGAGGAGATCAGGCCCAAGGAGGTTTACCTGGACCGAAAGCTGCTGACGCTGGAAGACAAAGAACTGGGCTCTGGTAATTTTG
GAACTGTGAAAAAGGGCTACTACCAAATGAAAAAGTAAGTTGCTATGTTCCACCAGCCTCACCCTCACATGAGCTCAGGTTCTAGACATGACTG
AGAATAACAAAATGTAACCTGGCATGACTAAACCCTTTTTTTATTAATGCTACCATTCTTGTTAATTTCTGGTACTATTATAATGAAAGCGTCC
ATTACCTTTAATGGCAAAAACTGCAATTACTTTTGCACCAACCTAATAGTACATGCACCTTATGGAAAATTAGGAAATAGAAACAAGCAGAAAC
ATAAGAAAATAAAAACATCAAATTGCCATGGTGGCTCATGCCTGTAATCTTAGCAATTTGGGAGGCTGGGATAGGAGGATTGTTTGAGGCCTGG
AGCTTGAGACCAGCCTGGGCAACATAGCAAGACCCTGTCTCTACAAAAAAAATAAATAAATAAATAAATAAATAAAACATAAAAAATTAGCCAG
ACATAGTAGTGCACACATATACTTCCAGCTGCTCAAGAGGCCAAGAAGCCAAGGCTGCAGTGAGCCATGATTGCACCACTGCACTCTAGCCTGG
GTAACAAAGCAAGGCCCTGTTTCAAAAAACAAAACAAAAAAAAGAAAAGAAGAACATCATATTTGTATAATACCATCCCAACACTACTACTGTT
AACTAAACATTACTACTGTTAACTTAATCTTATTGTATATATTTTTAGATCTTTATTTGTACACACAGATATTATTTAATTAAAACAAAATCAT
TCCATTATAACAATAGTTTTTACATTTATTTTCTTTAATTTTATAGTTACATCTCATTTTTCTTACATGAAAATTATTGATTCCAAATATGATT
AGTATAATTATTTATTGGCTTTATCCTATGATTTCAGATAACAATACATATTATACAACAGTTAAAATTGCTAAATTTCGAAAATATCTTTGTA
TTTCTTTTGGTCTTTAGAAGGTACCCAACATAGAATGTACAAGTAAAACTACTGGTTTCTAAAGTCCTTTGAAATACACCCTCTGTTTGAATTG
GTATGATACTAATATGAATGTGGTTAGATTTAGTAGCTTCATGGTATTTCAAATGTAAGGGATTTTTGTAAAAAAAAAATTCGTGAGTAGGTAA
TTTTTATGATTCAAGGGTTAAAAATATAGACAAAAATATACACAGACATGCGTGCTTTCCGGCCGGGTGCCGTGGCTCATGCCTATAATCCCAG
CACTTTGGGAGGCCAAGGTAGGTGGATCACCTGAGGTCTGGAGTTCAAGACCATCCTGGCCAACATGGTAAAACCTCGTCTCCACTAAAAATAC
AAAAAATTAGCTGGGTGTGGTGGCATACACCTGTAGTCCCAACTACTTAGGAGGCTGAGGCAGGAGAATCACTTGAACCTGGGAGGAAGAGGTT
GCAGCCAGCCAAGATCACACCATTGCACTCCAGCCTGGGCAACAAGAGTGAAACTCTGCCTCAAAAAAAAAAAAAAAAAAAGAAAGAAAGAAAA
AAGAAATGCTTGCTTTCCAATCTGTCCCTTCGATTTGTTTTTGTGCATGTGTGTGTGTGTAAATTAGATACACATACATGTATGTATACACACA
CAAACATACACACATATGTATACATTCACAAGTATATACATGCGCACATATATACACACACAATCAAAATTGGCTGTGATACTTATTTTAAAAT
ATATGTTTGTATATCACAGTCATTTTGTACAATGAGTAAAAATGTTTCTGTATTAATCCTAAGCTTTTTCAGATTATGAAAGGTTGTGATCTTT
ATTCTGTGCTTAAATTTGTGAATTAACATAGACATGACTGTGGACATAATTTCAACTAGAGAATTTGGGGAGAGGAGAGGCTCTCTCGGTCAAT
TTGGGGCAAAATGATGTCTTTACAATACTGAGTCTTTCAATCTATAAACACAGCCCATCTCTCCATTTACCAGGTCTTCTTCAGCCTCTCTCAG
GAGAGTTGTTTACCTTTTCAATGTATTACATTGAAATATATGCTTTTAAATTCTGCACCTAGAAATTCACTTTCAACAATTATGAAAGGCATTT
TATATTCACATTCATTACATGTAATCTGAGATGGTGAATCCCAAGATGTTACAGGAATTCGTACATGTCATCGTTGGTGAAGTTTATTTACAAG
TTATTTAAATATTTTACAGTTTAGGGTTTTTGCTTGGTTGGCACATTCTTCAGTACTGTTCTTCACCATTCTTTTTATTTTATATTATTTTTTA
GAAACAGTGTTTCACTGTGTCACCCAGGCTGGAGTGAACTGGCAAGAAGCTCACTGCAGCCTTGAACTCCTGAGTCCAAGTGATTCTCCCACCT
CAGCCTCCCGAGCAGCTGGGACTACAGGCTTGCACTGTCACGCCTGGGTGATTTTTTTTAGAGATGGGGGTCTCACTATGTTGCCCAGGCTGAC
CTTGAACTTCTGACCTCAAGCAATCCTCCCCTTTCAACCTCCCAAAGTATGGGGATTGTCAGCATGAGCCACCATGCCCAGCCCCCACCATTCT
TTTTTAGTAATGGAGTATTACTCTCATTGTCTATAAAATAAGGGAATATAGGTTGGAAGGCCAGCCATCCATTACAGGCTTCTGAAGACACATT
GCCACTCTGTGGCAGCTATTTCCGTGGGACTGTTTCCACAGGGCGATTATGCTTCACAGGATAAGATTATCTAGAAGTGAGGCATTTTGGAAAG
TTTCTTGTGTGTTATGATTTCTCTTGCAGAGTTGTGAAAACCGTGGCTGTGAAAATACTGAAAAACGAGGCCAATGACCCCGCTCTTAAAGATG
AGTTATTAGCAGAAGCAAATGTCATGCAGCAGCTGGACAACCCGTACATCGTGCGGATGATCGGGATATGCGAGGCCGAGTCCTGGATGCTGGT
TATGGAGATGGCAGAACTTGGTCCCCTCAATAAGTATTTGCAGCAGAACAGGTATTGTCAGGTGCCCCCACACATCTGGAAGCTATCCCCTAAG
GGACAGGGCCCACCCCTGGATGGGCCGAGCAGCCTGATTTCCTTGGGAAGCCTTCCCACCCTCCTGTGCCTTCCTTTATTGGTCCATACTCAGG
GACCTTGACAGAGGGACAGGGAGACATAGTCTTTGGATTCAGCTCTGTTAGCTCTAGTTGTTCAGAGAAGAACTCATTTGACAGTTTTGTCTTT
TTTTTCTTCCTGTAGTCATTCAGTCTTGGCCTAGTGATAGACTCAGCCTCAGAATATCTTGACATGGAATCTCCTTTCTCACTGGGAAGAACAC
TTTCTTTTTAAAGAAACATTTTAAATAAATGCTTTTCTAAGAGGTGACAGTTAAGTCTTATCTGTTTGGCTGACCTTATGTTCTCATGAGTGAT
GACTTCTGAAAATACCAATCACTGCTAATTGATTACATCCATCAAGGGAGTAATTGTGTTCTTCTCATCTAAAGCTGTTGTTGAGATGAAGAAA
CTCACTTGGAGTAAGACAGAATGATGGCCACCGAGGAGCTTGCCTCATTTGCTGTGCTACGTTTGTACTTAGATTGTCTCTTTAGTCTCCATGG
AAGGGAGGTGTTCTTACCCTGACTTTGTAGGTGAGAAACTGATTCTCAGGGTTAGTTCTGTGACCTGTGTGATACTGGAACAAGGATTTGAGGT
GGTGCCTGGCTATCTGTGAGCCAGGCCTGACTACTCTGTTATACTGCTTTTCTTGCAATTCCAAGCATGCCAGGAAACTCAATCCCATTGAACA
CCAGGAGAATATGGCATTGAGTCCTGAAGAGCTATATGCTAACAAAGTTATTTGTCACCACTCGTGAGTTTTGTTGTTTAAGAAGAGGGAAGGA
GTAGATTCACCCACTGCCTGCGTGAGGAGCATGGTTGTTTGTTGTGAAATGGGTCACTGTCTGTTATAGCTGATGAGATCATTATGACTTTCAG
ACATGTCAAGGATAAGAACATCATAGAACTGGTTCATCAGGTTTCCATGGGCATGAAGTACTTGGAGGAGAGCAATTTTGTGCACAGAGATCTG
GCTGCAAGAAATGTGTTGCTAGTTACCCAACATTACGCCAAGATCAGTGATTTCGGACTTTCCAAAGCACTGCGTGCTGATGAAAACTACTACA
AGGTAAGTACAACTTAGCTAATATTCAGAGCAGCAGGTGGTAAAAAATGCAAGATAAATGTACGTTTTCTTTATTTTATTATTGGTATGGTTTC
AAAAAGCAGTTTTGCTACTGAAAAATAACTATGTAAGATATGTTCTTATAATCTTTGATTTTACTGTTTAAAAAATAAAAATAATATCACACCT
TGAGATTTACTCAAAGTAGTATATTCTAAATTATGTTTCATGGAATGATCAGGAATCGAGGTATTAAGTGAAGATTTGTTTTGAAAAAAGAAGA
ATGGGATTCCTTCATTAAGTTAATTTGAAAAGTTTGCATTGGACAGCTTGATTCTTGCAGGATTTCCCCGAAGCCCTTTTCATGCTACTTAACA
TTACGAATCTCAGTAGAGGGTTTACTGCACTGCATTTCTCAAACCAGATTCATCCATGGCATCCTTTTCTGATAGGACATTTCATAACATGTGG
TGGAACACTGTCCCACAGCATGGAGCTATTGGAGCTTGGAGCTCCAGTACTGCAGTTCAGAGCTCCAATATTGCAGCTTGGAGCTATTCCAAAA
TAACTTTCTAATGAACACCTACCTATACATGCAAAAGTTAGAAAGTGAAGATTTACCAAATAAACTCGTCATCTGTGTATGTCCACTACATATG
TAGAAATTGTTCTATTGGCTTAACTGGTATCCAGAAGAGTTGAAGTGTCAAAAAATAGATGCAGTGCTGGAAAGAAGAGATCTAGATAGTTATG
TGACTGAAGCTAAAAGAAATATGCCCCTTGGTTTGGTCAGGCAGAGATTATCAAGAATTAAGGGTGGACTTCCAGCAAAACATAAAGTAGAAAC
TAGTTCTGGGTGCAGGTTTGGACTTCCTAAAAAATAATTTCACAGTGACCAATCCCCTTGCTAATATAACTTGACCCCTCTCTGAACCCTCAAA
TGAACCAATAACAACTAAAAGAAGAAAAGATAAGAAAGCTCTATCTTCATATTAACTCATCTGTGGTGTATACCTAGGGTCTATGTTAACCAAC
TGGAAAACGTGATGGCACCAATCCCACTGAGTCAAAGAGGGTGCCGAAGGCAGGTTAACAACCCTGCTAGTCTCTGGTGAAGTCCACATGCTCA
GAAATAGATGGCAGACCTTCAAAATCTACCTAGTGCGTGGCAGATAGGATCCTGTGTTTCATGCCCTGCAGTGGTCAGAGACCCCACCCTGAGT
GACAATACCCACCCACTCACCTTTCCCTAAGAGGCAGTCCATGGACTGGTAGGTGATGGGGAATGGTGGACCTCGGCCAGCAAAGGGACTGCAT
GAAGGGTAAGCCACAAGAGCCAAGCTCCCAGGCAGGCAGGCAGACTCCACCATCTTGAGCACAAAGGCCAGGCGAGAGTACAGCAGGGGTCAGG
CTGGCCCTCCTCACCCCATTCTTCATACAGTAGCTTCCCAGAAGGAAGCCCCTTCAGAGGTGAACCACTGTGAGAAAGGCATCAGCATGGGCCT
GGATAGAAATAGCAGGGAAACAGAGCAAGGAAGACAGCAGCCTAGGATGACCAGCATGGGACATGCTGGAGAAAGAAGCCTGTTGTAATGCTGG
ATGTACCTCATCATGATCATTACAGAAAAGAAGACAGGCTGTCTGAAACAAGGACACAGAGGAGATAGAGAAAATGAAACATGAATGCTGGGCA
CCTTATCCACTGCTGTATAACAAATCACCCCAAACTTACTGCCAAAAACACCACCAGGTGTTGGTTTCCCTGCCGTCCTGCTGTGTGGGCAGGG
CCAGGTAGGCAGGGTTCAGTGCGTCTGCTCACACAGGGGATTGCACAGTGGCCCGAGGGACAGGAGGACCGTTTCCACTCTGCTCACTCACACT
ACTCAGCCACTGAGCAGCTGGTTAGACACAGCCCATGCTGGAGACCTCTGCGGGGCTGTCAGCCAGCTCACCTGCACATGGCCTCTCCTTGGGG
CCCTTGGGCTTCCCTTGGGCCCATGCTTCCCTTGGGCAGCATGGCAGCTGCGGGCTGAGAGCAGTTACCCAAGAGGCAGGAAGTGGGGGCTGCC
AGGCCTATAAAACCAGGCCCAGAAACTACCTCAGGGTCATTTCCACCTGATCCTATTGGTCAGAGCAGCCAAGGCCACCAGCTTCAAAGGATGG
GATAGATAGAACCACCCTTTTGAGGAGAAGTGTCAAAGAGTCAAAGAACATGGGGCCAATTCTAAAACTGCTGTAGATATTAAACAGCATACCA
TTAAAAAGTAAACTAAACTAAAAAGTAAACCAAAGATTGTCTTTGGACAATCAATGAAGAAAAAAAGCCACTAGGGAAATAAAAGCCATCTTGG
AAGCAGCAAAAAGAGACAAAACTACCGAAAAGATAGGGTAGTGGAAGATTTCTGTATGAAAACAAAGCAAAGTGAAATGGAAAAGAATAAAAAG
TGGGCCGGGCGTGGTGGCTCACGCCTGTAATCCCAGCACTTTGGGAGGCTGAGGTGGCTGGATTACCTGAGGTCACGAGTTCGAGACCAGCCTG
GCCAACATGGTGAAATCCCGTCTCTACTAAAAACACAAAAATTAGCCAGGCATGGTGGCAGGCACCTGTAATCCCAGCTACTCTGGTGGCTGAG
GCAGGAGAATTGCTTGAGCCCGGGAGGTGGAGGTTGCAGTGAGCTGAGATCGTGCCACTGCACTCTAGCCTGGCCAACAGAGCAAGACTCTGTC
TCAAAAAAAAAAAAGGAATAAAAAGTGAAACAAGATCAAAGAAAAGTGCTAGCTGTGGAAGCAAACGAACGCTGGCACTCGGGAAACCATGCGT
GACAGGGTAGATTTGTGCAGCATCACAACTGAAAAACACAGGTGTTCTTTGATCCAGCAGCTCCTTCCGAGAGCAAACACACACATCTGGTAAT
GTGCACTCTAGCACGGGCTGCCACGGCAAAAATCTGGAAACTGCCTGACGACTGCTCCGCAGGGTTTGGTCCAATCAGTCATAGCAGATTCACA
GGAAGCTCACAATGCAACCCTTTTAAAAGTGACTTATTTGCATGTGCTGACCTAGAAATATGTCTACTATACATGGTTAGGTAGAAAAATTAAA
AAGTACAGTGCTCAAAGAATATGCCCCTAGTTTTATGACCAGAAAAAAGTATACACTACCTATTTATGCATGTATCTAGGTTATTCTGAAGACA
GCCTAAATTTCATTGCATTTAAGATTTGTCAAAGGTTTACTTTTTTCATCAGTTCAAAATTCAGAAGAAACCTGAAATAACACACATGGTTGTA
TTGTTCATGACTTGTAAGATCAAAAGTTTAAAATAGCACACAAGGTGATGGAAACAGAAGTATAATCAGTTGTATCATTTGGAAGAGAAGTGCC
AGGGCACCTTATGGTCTCCAGATTGATGTCCTGAATGTCTCCTTGTGATCCACTGTAGTGTCATGAAATCAGTTTTCACTGAAATCATGTCCCC
CTCTCCACCCCAATGAGACCTCACACAGGTGACATCAGCAGAAGATAACCAGACTCCGCTGTGTGCCCCCTGAATCCCAAACCCAGAGAGAGGG
TTTTGCTATGCAGTTTTGACCTCTGTACTATCACTTGGTTTTTAAAATTAGGAAATTAGAATGCTTAGATCATTTTTTAATGATGCTAGCAAGT
AGTGCATGCTAAAATAATTGACCTGGGTGGAGAACTGATCAAGATGGACGACTAAATGCAACTAGTATATGCTCCTCCGTAGAGACGGACCAAA
ATAACAAGTACATATTCACACTTCCAATAGATGGTCTAAGAGAGAACACAGATGCAACCAAGGAGCGACAGGAAGCACCAAAAGCAAAGCACAG
GGAAACAAGGCAGCCTGCTTGGCCAGGAATGACTGGGACCTGGGAGAAGCTTCCAGATATGGGGAAAGCATAAATGAGAGATCCCCAGGGCTCC
ACATTCCAGCCAAGGACTTCTACAATCCTAGCGACAGAAGAACCCCTCGACCCACGTGGGCCTTAAGACTAACATAGGGAGCTGCCTAGGGATC
ACACAGAGGCATTGCTCCATAGAGGGAACTCACGCTGAGTCCCACAGGCTTCCAAGCTCTGAGTAGCTGCAGCTTGGTGCCATACTGAGAACCA
AGTCCCCAAAGGACTGCATCTTGCCCTGGGACTATTGCTGATGCTGCCATCACCAGGCCAAGGACAGAGGGGAGAATGGACACCTTCACACACC
CCAGGGACAGATCCCCCTGCTGCTACAGGCTGCTGTGGGGCTGAGACACAAGCAAATCACACCCCCTATAGCTGCCTGCCTACACTGCTCCCCC
TGAGAATGTCCGCAGCCTCCCCAGGAGCAGGCCCATAGCCAGCGCCATTCTGAGAGCCAAGCCTCCAAAGGTCTGTGTCCTTCCTTGGGCCAGG
GCTGATGCTGCCACTGCCCCGCCACATAGGGAGAAGGGAGGCCAGGCACTTTCACATGCCACAAAAACAAATACCACCACCACTGCTGAGGGCT
GCTGTGGGACCAAGGCTCAAGAAGACTGCATATCCCACAGCTACCTGCCTACTCTGCACCTATTGAGAGTGGCCCTACCCTTTCTGGTGGTAGG
CCTTCAGGGCAGCCACTGCTGGCAACCTAGAGACCACCCCTCCCTGCGTATCACAGCCAGCAGCTGAATGCACCACCAGGGAGCCTGAGCACAA
GTCCATTGGCCTAGTCACATGCCCCAATACTTGAGCATGACATCCTGTTGCAGCCACCGCCAACACCAACATGGACCACTTGGGTTCCAGTGAG
TTGCCCTACCACCATTACTGCCATCACCCACATCACACCCACTCCCCAGGAACCCAAGAACCTGCCCACATGCCCAGCCACTGCAGCATCCACA
CAATCTACCTGAAAGCCCAAGGGTCAGCCTTCTTAGACCTACTAACACCAGTGCTAGTGTATACCGCCCTAGGGCCCAAGGACAGGCATACTCA
GCTCACTGCTGTCACCACTGGGGTCTGAAGACTGGCCCACCTAGCATCCTATTCACAGTACACCTTCACAACAGCCTCCACTAATAACCATACC
CTAAGGCACTGAGAAAATCACAGGTGTCACTGAGGCTGTTTATGGCCATAGAAATTATACATAGATTAAATACTCCATTCACCCCAAATCAAAC
CCAAAGTGCCCTACATATATATATGTGTGTGTGTATATATACACACATACACATACGTGTATATATACACACATACACATACGTGTATATATAC
ACGCATATACACATACACATACGTGTATATATACACATACACATACACATACGTGTATATATACACACATATACACATACGTGTATATATACAC
ACATATACACATACACATACGTGTATATATACACACATACACATACGTGTATACATACATACACACATACGTGTATATATGCATACATACACAT
ATGTGTATATATACATACATATACACATATGTGTGTACATATACATACACATACACATATGTGTGTACATATACATACACACACATACACATAT
GTCTACATGTACATACATACACATACACATATGTGTACATGTACATACATACACATACACATATGTGTACATGTACATACATACACATACACAT
ATGTGTACATCTACATATATACACATATACACATATGTGTACATGTACATATATACACATATACACATATGTGTACATGTACATATATACACAT
ATACACATATGTGTACATGTACATATACACATATACACATATGTCTACATGTACATATACACATATACACATATGTGTACATGCACATATACAC
ATGTGTACATGCACATATACACATATGTGTACATACACATATACACATATGTGTGTATATACATATACACACATATGTGTGTATATACATATAT
ACATATATACATATATATACATATGCACATATGTGTATATATACATATATATACATATGCACATATGTGTATATATATACACACATATATGTGT
ATATATATATATACCCAACATATATATACCCAACAACATATATATATTTTCAGGAAAAAGACCTCCTCTACAAAAGCAATTTCAAAAAACTGGA
AGAAATGACTGTTGTACCACATGGGCAGATATCAATGTAAGAACACAGGAAACATGAAAAAGCAAGGAAATATGATACCTCCAAAGGAACACAC
AATTCTCCAGCAAAAAAATCCCAATCAAAAAGAAATTCACAAAACCCCAGATGAACAATTCAAAATATTGATTTTAAAGAAGCTAAATAAGATA
TAAGAGAATTCTGAAAAACAATAGAGAGAATTCAGAAAAACAGTTCAGGATATGAATGAGAAATTTATCAAAGAAATAGATATTTTTAAAAGAA
CCAAACAGAAATTCTGGAACTAAAGATTCATTGAATGCAATAAAAAATACATCCCAAACTCCACCAATAGACTAGATCAGGCAAAAGAAAGAAT
CTCAGAACTTGAAGACAGGTCTTTTGAAACAACCTATTCAGACAAAAATAAAGAGAAAAGAATAAAAAAGAATGAGCAAACCCTTCATGATATA
TGGGGCAGTGTAAAGCAACTGAATATTCAAATCATCAGTGTCCCCAAGGGTGAAGAGAGAATGAAAGATTAGAAATCCTATTTAATGAAATATT
AGATCAAAACTTCCCAAATATAACAACAAACCTAGACATCCAGATGCAGGAGGCCCAAAGGTCCCCAAACAGATACAATGCAAAATCATCTTCT
CCACAGCACGTTATAATCAAACCACTTAAAGTCAAAGCAAAAGCAAATATACTAAAAAGAGCAAAAGAAAATCTTCTAATTACCCATAAAGGAA
CCACCATTAGACTAACAGTGGATTTCTCAGCAGACACCTTACAGGCCAGGAGAGAAGGGGATGACATTTTCAAAGTGCTGAAAGAAAAATACTG
CCACCCAAGGAGACCATGTCCAGCAAAACTATTCTTCATAAATAAAAGAAAAATAAAGCCTTTCCCAGGTAAGCAAACACTGAGGGAATTCATC
ACCACTAGACTGATCCTAGAAGAAATACTCAAAGGAATCTTAAACGTGGAAGTAAAAGGACAATATTTACCATCATAAAAACACACAAAAGTAT
AAAACTCACTGGTAAACCAAACATACAAATAATCCTGTTAAATAGTGGGCTAAGAACATAGATAGACATTTCTCAAAAGAAGATATACAAATGG
CCAACAGGTATATGAAAAAATGCTCAATATCACCAAACAGCAGGGAAATGCAAATTACAAACACAATGAGATACCATCTTACCCCGGCTAGAAT
GGCTGTTATTAAAAAGACTTTTTTTAAAAAAATGCTTTGCAGATGCAGGAAAAAAAGGAACTCTTATACACTGTTTGTGGGAATGTAAATTAGT
ATAGTCACTACGGAAAATATTAGAGAGACTTCTCAAAACAAAAACAGGCTGGGCACGGTGGCTCACACCTGTAATCCCAGCAGTTTTGGAAGCC
CGAGGCGGGCAGATCACAAGGTCAGGAGATCAAGACCATCCTGGCTAACACGGTGAAACCCTGTCTCTACTAAAAATACAAAAAATTGGCCAGG
CCTGGTGACAGGTGCCTGTCATCCCAGCTACCTGGGAGGCTGAGGCAGAAGAATGGCATGAACCCAGGAGGCGGAGCTTGCAGTGAGCTGAAAT
CACACTACTGCACACTCCAGCCTGGGCGATAGAGTGAGACTCCATCTCAAAAACAAAAAGAAGAAAAAAAAACTAACTACACATAGAATTACCA
TATGATCCAGCAAGTCTACTATTGGGTATTTATCCAGAGGCAAATAAACTTGTATCAAAGGGACACATACACTCAACGTGCTTATCATAGAACT
ATTCACAATAGCAAAGATGTGGAATCAACCTAACTGTCCATGGATGGATGAATGGATAAAGAAAATGTGGTGTGTATATATATATATACACCTC
TAGAGGTGTTCACTGGTTACTTAGGGTACACCCTATGTAAATGAAGAGGCTAAAGTGAAGTTACAAAGTTATTTACTTGGTGTACACCCTATGT
AAATGAGGAGGATGAAGTAATGTTACAAAGTCATTTACTGAGTGTACACCCTGTGTAAATGAAGAGGATATTTCCTGTCATAGCTGAAGTGTTT
CCATTTGATTTAGTTCTAGGAAGTCCTTAGGTTCCCTGCCTCCAGGCCCTATTCTCCTGTCTCAATTTCACTCAGCATAATGTCCTCTAGGCTC
ATGCATATTGTAGTGAATTCCCTTCCTTTTGAAGGCTGAATGCTATTCCATTGTCTATATAGAGGGTGTTTTGTTTATCCACTCATCTGCTGAA
GGACACTTGGGTTGCTTTCATGTTTTAGCTATTGGGAATAATGCTGCTATGAACATGAATGTACACAATTTTATGCTTTTCTTTCTTTCTTTTT
TTTTTTTTTTTTGAGATGGAGTCTCTCGTTGTGTCACCCAGGCTGCAGTGCAGTGGTGTGATCTGAGATCACTGCAACCTCTGCCTTCTGGGTT
CAAGCAATTCTCCTGCCTCAGCCTCCCAAGTAGCTGGAATTACAGGCACCCGCCACCACACCTGGCTAATTTTTGTATTTTTAATAGAGATGGG
GTTTCCTCTTGGCCAGGCTGGTCTCAAGCTCCCGATCTCAAGTGATCTGCTCTCCTCAGCCTCCCAAAGTGCTGGGATTACAGGCATGAACCAA
TGTGCCCGGCCCACAATTTTATTCTTAATGGAATTTCTCCCTCTGCTTTGCTTTTAGGCCCAGACCCATGGAAAGTGGCCTGTCAAGTGGTACG
CTCCGGAATGCATCAACTACTACAAGTTCTCCAGCAAAAGCGATGTCTGGAGCTTTGGAGTCTTGATGTGGGAAGCATTCTCCTATGGGCAGAA
GCCATATCGAGTGAGCCAGTCCTGCTTCATTTTCTCACTGTGGGGCCATTAGAACAGATAAGCACCAGATTGTCTTTACAACAACCTGAAAATC
CTAATCTGAGTCTCTCATTATCTTTTACCAGTAAGCAGTTAATGTGCCCTCAGTCTATTCCAAACTGAGAATGAAATATCTGATGCTCACTGAA
GAGTGTGTTTTTCCCATTGTGTGTCAACACATGTAAATATACACAAAATATAACCCAGATCATTACTCATTAGAGTTGAAATAGAAATAATGAC
TATTTACAGTATCTTAATTACATTTTTTAGATCTTTTAATTAAAACATATATACCAAAAAGGGCACAACTCTTAGCATGCAGTACAGTGGTTTT
CCACAAATGAAACATACCCATGAAATCAGCCCCCAGATCAAGAACAGAACCCCAGAAGCCTGTTTCCTCTGCACACACAGCCTTCCCCAGGGGT
GACCCAACTGACTCCAACATCACAGATTAAGTGCTCCTGTTTTTATACTTCGTATAATTGAAATCATACAATGTGTACTCCTTCATGTCTTGGA
GTGGCTGTTTTGTTTTGTTTGACTAACACCTTTAAAAAAAAAAAAGCTTATGCATATCTTGCATGTTGTAGGGGATGAAAGGAAGTGAAGTCAC
CGCTATGTTAGAGAAAGGAGACCGGATGGGGTGCCCTGCAGGGTGTCCAAGAGAGATGTACGATCTCATGAATCTGTGCTGGACATACGAGTGA
GTACCTGACACTGACTGTGATGTATTCAGATGCTCTGGAAACAGGTGAAATGGTTGGGCGTCTAAAAAAAAGGATAAAGTTCACCTTTTCATGA
ATCAGGAATGTGCCCGCTTTCTAAACAAACAACGGTGCCGGCTCGTCTTGCTTTAGAGACCTTGCTTTCTGGCCCATGCACTTACATGTAGTAG
AGCCTAAAGAGCAAGTTTAGATGGGGACTCTAACCCGAGTGTCCAAATTTAAATGTCGATAATGGTGGAGAGATGGGGCAAGGGCAGTCAAAGT
AAAAAAGTGTTTGTAAGAGTTTGGGGCTGGATGCCATTTTTGGCATAGAGGATGGCAGAAGCACTCGGTAGGGGATGGTATACTTTCCTGACTC
CCTGATTACCATGAGCAGTGAGGGCTGACCTCGGACTCTGGCCAGCTCACAGATACATTGGCTAAGGTTGCACTTCCCTAGAATCTACCTCATC
CTTGAAGTCGTGGCCTCAGTCCACCCTGACTGCCGTCATACGTCTCGCTTAGTGGATCCCAGGTGCTGTTATGCTTTTAAAGTACACCTGGCAT
TATTCTTAGGTGATGTTCAGGGCAGCTCAGCAAGGAAGGAAAATGCATTGCCACTGAAAATTAGGAGTACAGTTTTGCCAGGCTCAGCAGCCTG
CTTGTGCGTGCATAGCACGGTGACCCCAAGCCGCCATCTCCAGTTCCCTGGTACTGAACTGTGCACACAGAGCCCACACAGCTGAGAATCGATG
TGGTTTGTGATCAGGAGGACATCACAAATCCTGGCCATGGATTTGATGCTTTGCGGAATCAGGTTTGCCTTCAGATACAGCTGACATTGCCAGA
GCCTCTGAAAGATGAGGGCTATTCCAAATGCTGCAGATGCCACCTGGGATTCCGAGACCTCCCCTCCCCACCCTGGGAGTATCACTCTACCACA
ATTGTGAGTGACAGGGAAATAATCTCATTCTGTATCTAGAAACCACCCAGGGGGCAGCCACAAAGGCAGCAGGCAAACTGTGGGTATATGCTTC
CCTGCCCCTGCTGCTCCCGTCGCTCAGGAGAGGAGCCTGGCCTGGGCCCCGGAGCATCTTCTCCAACCCCAAGTGTTAGTGCAACAGGGAGAGC
TAAGTGTCAGCAAGAGGGAAGTGCTACTCATGGATCTAAGAGGATGTTTATTCCTAAGTCTTGGCTTAATTCTCCCAGTGCCTAAACTAGAATC
GTAAGAACCATTGGCAGGTACGCACTGGCAGCACTGGACCCTGGGGAGGGCAGCCCTTGGGCACGTGGCAGCCTATGACCCTTTACTGAAAGTG
TTCTTTGGTCCAGCCTGCAGCTCCCTTTCCAAGGCTTCCCTTCTTGTAGAAGGGTGA
HUMAN SEQUENCE—mRNA
GAGGAAGAGCCGCGGGCCCGGCGGCTGAGGCCACCCCGGCGGCGGCTGGAGAGCGAGGAGGAGCGGGTGGCCCCGCGCTGCGCCCGCCCTCGCC
TCACCTGGCGCAGGTGGACACCTGCGCAGGTGTGTGCCCTCCGGCCCCTGAAGCATGGCCAGCAGCGGCATGGCTGACAGCGCCAACCACCTGC
CCTTCTTTTTCGGCAACATCACCCCGGAGGAGGCAGAAGATTACCTGGTCCAGGGGGGCATGAGTGATGGGCTTTATTTGCTGCGCCAGAGCCG
CAACTACCTGGGTGGCTTCGCCCTGTCCGTGGCCCACGGGAGGAAGGCACACCACTACACCATCGAGCGGGAGCTGAATGGCACCTACGCCATC
GCCGGTGGCAGGACCCATGCCAGCCCCGCCGACCTCTGCCACTACCACTCCCAGGAGTCTCATGGCCTGGTCTGCCTCCTCAAGAAGCCCTTCA
ACCGGCCCCAAGGGGTGCAGCCCAAGACTGGGCCCTTTGAGCATTTGAAGGAAAACCTCATCAGGGAATATGTGAAGCACACATGGAACCTGCA
GGGTCAGGCTCTGGAGCAGGCCATCATCAGTCAGAAGCCTCAGCTGGAGAAGCTGATCGCTACCACAGCCCATGAAAAAATGCCTTGGTTCCAT
GGAAAAATCTCTCGGGAAGAATCTGAGCAAATTGTCCTGATAGGATCAAAGACAAATGGAAAGTTCCTGATCCGAGCCAGAGACAACAACGGCT
CCTACGCCCTGTGCCTGCTGCACGAAGGGAAGGTGCTGCACTATCGCATCGACAAAGACAAGACAGGGAAGCTCTCCATCCCCGAGGGAAAGAA
GTTCGACACGCTCTGGCAGCTAGTCGAGCATTATTCTTATAAAGCAGATGGTTTGTTAAGAGTTCTTACTGTCCCATGTCAAAAAATCGGCACA
CATTCAATCCGTATGAGCCAGAACTTGCACCCTGGGCTGCAGACAAAGGCCCCCAGAGAGAAGCCCTACCCATGGACACAGAGGTGTACGAGAG
CCCCTACGCGGACCCCGAGGAGATCAGGCCCAAGGAGGTTTACCTGGACCGAAAGCTGCTGACGCTGGAAGACAAAGAACTGGGCTCTGGTAAT
TTTGGAACTGTGAAAAAGGGCTACTACCAAATGAAAAAAGTTGTGAAAACCGTGGCTGTGAAAATACTGAAAAACGAGGCCAATGACCCCGCTC
TTAAAGATGAGTTATTAGCAGAAGCAAATGTCATGCAGCAGCTGGACAACCCGTACATCGTGCGGATGATCGGGATATGCGAGGCCGAGTCCTG
GATGCTGGTTATGGAGATGGCAGAACTTGGTCCCCTCAATAAGTATTTGCAGCAGAACAGACATGTCAAGGATAAGAACATCATAGAACTGGTT
CATCAGGTTTCCATGGGCATGAAGTACTTGGAGGAGACCAATTTTGTGCACAGAGATCTGGCTGCAAGAAATGTGTTGCTAGTTACCCAACATT
ACGCCAAGATCAGTGATTTCGGACTTTCCAAAGCACTGCGTGCTGATGAAAACTACTACAAGGCCCAGACCCATGGAAAGTGGCCTGTCAAGTG
GTACGCTCCGGAATGCATCAACTACTACAAGTTCTCCAGCAAAAGCGATGTCTGGAGCTTTGGAGTGTTGATGTGGGAAGCATTCTCCTATGGG
CAGAAGCCATATCGAGGGATGAAAGGAAGTGAAGTCACCGCTATGTTAGAGAAAGGAGAGCGGATGGGGTGCCCTGCAGGGTGTCCAAGAGAGA
TGTACGATCTCATGAATCTGTGCTGGACATACGATGTGGAAAACAGGCCCGGATTCGCAGCAGTGGAACTGCGGCTGCGCAATTACTACTATGA
CGTGGTGAACTAACCGCTCCCGCACCTGTCGGTGGCTGCCTTTGATCACAGGAGCAATCACAGGAAAATGTATCCAGAGGAATTGATTGTCAGC
CACCTCCCTCTGCCAGTCGGGAGAGCCAGGCTTGGATGGAACATGCCCACAACTTGTCACCCAAAGCCTGTCCCAGGACTCACCCTCCACAAAG
CAAAGGCAGTCCCGGGAGAAAAGACGGATGGCAGGATCCAAGGGGCTAGCTGGATTTGTTTGTTTTCTTGTCTGTGTGATTTTCATACAGGTTA
TTTTTACGATCTGTTTCCAAATCCCTTTCATGTCTTTCCACTTCTCTGGGTCCCGGGGTGCATTTGTTACTCATCGGGCCCAGGGACATTGCAG
AGTGGCCTAGAGCACTCTCACCCCAAGCGGCCTTTTCCAAATGCCCAAGGATGCCTTAGCATGTGACTCCTGAAGGGAAGGCAAAGGCAGAGGA
ATTTGGCTGCTTCTACGGCCATGAGACTGATCCCTGGCCACTGAAAAGCTTTCCTGACAATAAAAATGTTTTGAGGCTTTAAAAAGAAAAAAAA
AAA
HUMAN SEQUENCE—CODING
ATGGCCAGCAGCGGCATGGCTGACAGCGCCAACCACCTGCCCTTCTTTTTCGGCAACATCACCCGGGAGGAGGCAGAAGATTACCTGGTCCAGG
GGGGCATGAGTGATGGGCTTTATTTGCTGCGCCAGAGCCGCAACTACCTGGGTGGCTTCGCCCTGTCCGTGGCCCACGGGAGGAAGGCACACCA
CTACACCATCGAGCGGGAGCTGAATGGCACCTACGCCATCGCCGGTGGCAGGACCCATGCCAGCCCCGCCGACCTCTGCCACTACCACTCCCAG
GAGTCTGATGGCCTGGTCTGCCTCCTCAAGAAGCCCTTCAACCGGCCCCAAGGGGTGCAGCCCAAGACTGGGCCCTTTGAGGATTTGAAGGAAA
ACCTCATCAGGGAATATGTGAAGCAGACATGGAACCTGCAGGGTCAGGCTCTGGAGCAGGCCATCATCAGTCAGAAGCCTCAGCTGGAGAAGCT
GATCGCTACCACAGCCCATGAAAAAATGCCTTGGTTCCATGGAAAAATCTCTCGGGAAGAATCTGAGCAAATTGTCCTGATAGGATCAAAGACA
AATGGAAAGTTCCTGATCCGAGCCAGAGACAACAACGGCTCCTACGCCCTGTGCCTGCTGCACGAAGGGAAGGTGCTGCACTATCGCATCGACA
AAGACAAGACAGGGAAGCTCTCCATCCCCGAGGGAAAGAAGTTCGACACGCTCTGGCAGCTAGTCGAGCATTATTCTTATAAAGCAGATGGTTT
GTTAAGAGTTCTTACTGTCCCATGTCAAAAAATCGGCAGACAGGGAAATGTTAATTTTGGAGGCCGTCCACAACTTCCAGGTTCCCATCCTGCG
TCCTCCCCTGCCCAAGGGAACCGGCAAGAGAGTACTGTGTCATTCAATCCGTATGAGCCAGAACTTGCACCCTGGGCTGCAGACAAAGGCCCCC
AGAGAGAAGCCCTACCCATGGACACAGAGGTGTACGAGAGCCCCTACGCGGACCCCGAGGAGATCAGGCCCAAGGAGGTTTACCTGGACCGAAA
GCTGCTGACGCTGGAAGACAAAGAACTGGGCTCTGGTAATTTTGGAACTGTGAAAAAGGGCTACTACCAAATGAAAAAAGTTGTGAAAACCGTG
GCTGTGAAAATACTGAAAAACGAGGCCAATGACCCCGCTCTTAAAGATGAGTTATTAGCAGAAGCAAATGTCATGCAGCAGCTGGACAACCCGT
ACATCGTGCGGATGATCGGGATATGCGAGGCCGAGTCCTGGATGCTGGTTATGGAGATGGCAGAACTTGGTCCCCTCAATAAGTATTTGCAGCA
GAACAGACATGTCAAGGATAAGAACATCATAGAACTGGTTCATCAGGTTTCCATGGGCATGAAGTACTTGGAGGAGAGCAATTTTGTGCACAGA
GATCTGGCTGCAAGAAATGTGTTGCTAGTTACCCAACATTACGCCAAGATCAGTGATTTCGGACTTTCCAAAGCACTGCGTGCTGATGAAAACT
ACTACAAGGCCCAGACCCATGGAAAGTGGCCTGTCAAGTGGTACGCTCCGGAATGCATCAACTACTACAAGTTCTCCAGCAAAAGCGATGTCTG
GAGCTTTGGAGTGTTGATGTGGGAAGCATTCTCCTATGGGCAGAAGCCATATCGAGGGATGAAAGGAAGTGAAGTCACCGCTATGTTAGAGAAA
GGAGAGCGGATGGGGTGCCCTGCAGGGTGTCCAAGAGAGATGTACGATCTCATGAATCTGTGCTGGACATACGATGTGGAAAACAGGCCCGGAT
TCGCAGCAGTGGAACTGCGGCTGCGCAATTACTACTATGACGTGGTGAACTAA

TABLE 40
MOUSE NOMENCLATURE
ICSGNM Gnb1
Celera mCG23363
HUMAN NOMENCLATURE
HGNC GNB1
Celera hCG23470
MOUSE SEQUENCE—GENOMIC
AAGAGGGCACTAGATCTCATTATAGATGGTTGTAAGCCACCATGTGGTTGCTGGGAATTGAACTCAGGACCTCCGGAAGAACGGACAGTGCTCT
TAACCTCTGAGCCATCTCTCCAAGCCCTTTGTTTTTTGTTTTTGTTTTTGTTTTTTTAATTTCTGATATTAGAAATTTCTTTTATTTTTTAATT
AGATATTTTCTTCATTTACATTTCAAATGCTATCCTGAAAGTCCTCTATACCCTCCCCGCCCCCACCCTGCTCCCCAACCCACCCACTCCTGCT
TCCTCGCTCTGGCATTCCCCTCCTTTGTTTGTTTTTTGAGTCAGGCTTTCTCTGAGTCTCTGTAGACCAGGCTGGCCTGGAAGAGACAAAGAGA
TCTCACTGAGATTTTTATACACACTCAGAGAAAGAGACAGAGACACAGGGGTGAGGGTGGCTGGTATTAAAGGTGCTGGCCACCACTGTCCAAC
TAGGATGTGTTCTTAAAACATTCTAGGTACTGGTTGTTTATAAGGCTTGAGGCAGAATTGCTTGAAGATGGGAGATAGACCAGCAAGAAGGCCA
TGAGGCCTAAAGGGTGTTTTGGGGGTGGGGTGGGGCAGGGGAGAGGGTTGGTTGTTTTATATCAAGGCCCTTCCATTCCTAATTAGCAAGGCTT
CATTTAGGTCCCAGATAGCTTCTAGACTTGGAAAAAAAGTTTTTTTTGTTTTTGTTTGTTCGTTCGTTTTTTTTTTTTTTAAAGCAACAAGGGC
TCTTTTTTTTTTTTTAAAGATTTATTAATTATATGTAAGTACACTGTAGCTGTCCTCAGAGACTCCAGAAGAGGGCGCCAGATCTCATTACGGA
TGGTTGTGACCCACCATGTGTTTGCCGGGATTTGAACTCAGGACCTTTGGAAGAGCAGTCAGTGCTCTTACCCACTGAGTCATCTCACCAGCCG
TTCGTTTTGTTTTTTTTACCACTTGGACTACTGACCTAGGTGGGATTTGCCGCTGGGGTTAAGGGAGTAGGATGAGGTCTATAAAGAACTGAGA
GTAATAAGTGCTCCTGAGGCAGCAGAGCCACTTTGTTTCTAACGCTTGGGGTCTGAACATTGAAATCACCGTTTATGTGTGGTTTCACAGATTC
AGCTCTTTAAATGGACATGATACTTAGCTTTGAATCCTCTCCATTCTTGGGCTGGTGAGCAGGAAGCCGGCCTTTTCACTATAGCTCACTCTGC
CCCATAGCAGGTACTTCAGTCAGTCTGGGGGAGGGAAGGAAATTGTTTGTAGAGGAGAATTTTAAAAATAAGGCATTGCTGAATTTTTAAAAAG
AAGCATTACCTTTCTCAGGACTCAGCCCTGACAATTAGCTCCATTAACACACAAATACCTGTCTCCACTGCAAAAGGGTAGAAAAACTTCCCAT
GCTATTTGGTGAAAATTGAGTTGTGCCCAGAAACGATATCTTTGTTTCTAAGCACAAGTTGTACTTACAGTTCCTTCCCTTTGGAGAGGGCATG
CTGCCACTTTCATCTGACTTCAGGTGAGTTTCCAGAGGATCATACCTAGAACAGTGTTTAAGAGTTTCGAAGAAGCCAGGTGTGGTGGCGCACG
CCTTTAATCCCAGCACTCGGGAGGCAGAGGCAGGTGCATTTCAGAGTTTGCGGCCAGCCTGATCTACAAAGTGAGTTCCAGGACAGCCAGGGCT
ATCAGAGAAACCCTGTCTCGGAAAAAAAAAGAGTTACGTTGTTGCTGATAGGTACATATGCCATGTACATAAAACTTTCTTTCACCCACTTTTC
GAGTCATCCTGATACCTGGCCATGGCCTTCATGTAGTGTGAGAAAGTGGAGAGAAAGAGTGGGCAGCAAGCCAGGATGTTAAACTGTTTTGGGG
TGTGGGGTGGTGTAGTAAAATAAACGGGAAGTTTGACAAGAGAATTAAAAGACAATTCCTGAATATTAGTTTTTTTCACTTCTGAAACATGGAT
ATATAAGCCCCAAGATCCGCCATTTAGATGTTTAAACAAATTTGGATAAGTACCGAGGTTGCATCATTATGAAACTAACCAGAAGTGGCTGTGC
GCTCCAGTTGTACCGGAACAAAATGATGCTGGCACAGCAAGCTTCATGTCCTTTAGAACTTTATTATAAAGTTGGAGATGTTTTTTAGATACAT
TGGTATATTTGCATTATAGACTTAATGAATGTATGTAATGCCAAACAATGCTAAAATACTGACTTTTTAAAAAAAATATTAGTAAGTTAGGAGT
GTTGGTGGGTAGTTCAAGGTAATCCTAAACTACCTAGTGAGTTTGAAATCAATTGGGCTACATAAGACCTTGTCTTTAAAATATCAAAATAATA
ATTAAAGAAATGATTTAAACATACAGACTATATTACACTATATTACAAAGATCTCTCAGTTTATATTTACAGAATTCCGAGTTATATCTAATTG
CCTTTCTTCTCTTTCCTAATCCTTCTTTACTCTCTTGAAGTGGTTTTAAGGCAAACCCCCAAATCTTGCCCCCCCCCCCCCCGCCCCAATGAAT
AACAAGTTAGGAGTTCAAATCTGGTAGGATATCTAGCAGGAAAACAATAGTTTTCTTCTGTTTTCTGTCTGATTTTTCCCTTCTAAAAATGTAT
ATACAGCTGAATTTTTTTTTTTTTTTTNNNNNNNNNNNNNNNNNNNNNNNNNNNNNNNNNNNNNNNNNNNNNNNNNNNNNNNNNNNNNNNNNNN
NNNNNTTAATATGTACCCCACGTTGACCCAGTTCTCACAGTCCTCCTGCTTAGGTTTGTTTGTTTGTTTGTTTGTTTTTTTAAGATTTATTTAT
TTATTTATTATATGTAAGTGCACTGTAGCTGTCCTCAGACACTCCAGAAGAGGGAGTCAGATCTTGTTACGGATGGTTGTGAGCCACCATGTGG
TTGCTGGGATTTGAACTTCTGACCTTTGGAAGAGCAGTCGGGTGCTCTTACCCACTGAGCCATCTCACCAGCCCCTGCTTAGGTTTTTGAGTGC
TTAGATTAGAAGTGTGTCCCACCTCACCGAATTTGAAAATGTTAATTATTTAGGGAACTATTTACAAATAAATTCAACACTAGCTCATAGCTGA
ACTTCTTATATACGTCCCTATCCATTTCTCTACCTTTTGCTCGTTTTGAGGAGGCTTTTTAAAAATTAATTAATTTATTTTATATATGTGGGTA
CACTGTAGCTGTAGATGGTTATGAGCCTTCATGTGGTTGTTGGGAATTGAATTTTAGGACCCCTGCTCCCTCTGGCCCAAAGGTTTATTTATTA
TTATACATAAGTACACTGTAGTTGACTTCAGTCAGGCGCACCAGAAGAGGGCATCAGATCTCATTACAGGTAGTTGTGAGCCACTATGTGGTTG
CTGGGATTTGAACTCAGGACTTTTGGAAGAGCAGTCAGTGCTCTTACCCAGAGTCATCTCACCAGCCCCCAAGGAGACTTTTCTTAAATACTTT
TTTGTTCCTTTAAATATAATCGTAAACGGTACTATTATCACACTTTAAAATGTTAGCAGTTGCTTCTTATATAGCCCCTCAGCACACTTGTTTG
CTTGGGTTTGGCTTTATGGAGTTGGTCTAATCTTACACCTTCCTTCTTCCTCCCTCCCTCCCTTTCACATTGAGACAGTTTTTCATTTTGCAGT
CCTGTCTAGCCTGAAACTCACTATATAGACCAGGCTGGCCTTAGACTCACAGAGATCCAACTATTTTTGCCTCCCGAGTTCTAGGATATAAAGC
ATTGACCTACTATTCCCAGCCACGATCATAACTTTAAATCTTTTAGAAATTCTTGTCTTTGGAAATAGCATCTGAAAGCTTGGTGGTAGTGACT
GGTGGATACATTTAGTCCCAGAACTTAGGAGGCAGAGGTAGGCAGATATCTGTGAATCCTCTAGGCTAGCATGGTCTACAGAAGGAGAGTTCTG
GGATATCTAGAGCTATACAGTGAGACCCTGACTCAAAAAACCAAAGGAAAAAAGAAAGAAAAAAGAAAATAGCATCTCAAACTAGGACAATAGT
GTTCTTTGTCAATGTGACTTCTCAGATGTCAGGCATTAACCTTGGAGTTGTACTGTTGAAGGGGACACTTCAGTAGTATATCACTTCAGTAGTA
TATCACTTCAGTAGTATATCACTTCAGTACTATATCACTGAGAGGCTTATGGGATGAGGGCGTATCAGACTGATTATCTCACTATGTGATATAG
ATGATGTTTGTTTCTTTGTTTTTCAAGGCAGAGTTTCAGGTTAAAGACATGTACATCTCTACCCAGCTTCATAAATAAATAATATTTTTTTCAG
ACCAGAGTGAAAAGTGATTTAGTGAGGCCCTATTTGTATGTTGGAGAAATTGAAATTATTTTTCAAGACATGCACCATTAAATTTTAAATTCTA
GTTTATTTTTTGTATGTGTGCACACAGAGAAATCAGAGGACAACGTGAAGGAGTTCATTCTCCTGGGCAATCAAGTCAGGCTCTGGGTTTGACA
GCAGATACCTTCTCCCGAAGCCATCTTGATGGCTCGTATTTCTCCTTTTCATCAGTGGCAGGCTTGCAGCCCAAGTATCGTCAGTAACTGTGTG
AGATAATGGCGTCCTGAGGTCACTGCTGTGCCTGTCCTGGCTCCTTCACAGGGTCTTGACCAGAGGAGCGCTGTGCTGCTGATGCCTTTGTCAC
AAAGTTGGGGGCTTTATATCTCATACCATCACGTGAACTGTTCTAATCGTGCTGACATTTTATTTAAAGATATAGCATGTTACTTCCAGGAGAT
CACACCAACTTGTTTGTCACATCTTTAGTCCCATGTTCTTACATCATTTAAATTTCCTGAACAGCAGCTCTAATTTAAAGCTGACGGCAGATGT
GGGTCATGGGCAGTCGTGAGCCTCAGCAGGTGCTGTCTTAGTGGATTAGGCTGGAAAGTGCTACCTCAGCACAGCCAGAGCTTACCTGGGAGGC
TCTGGAGGAGGAGAGCTGCCCAGGCACGCAGACCGGATGCTGGGGTTTCCAGAAGGGTCAAAGGACTAACGACACTAAAGAGAAAGGCAGAGGA
GACGAAACGTAAGTAAGACGTGTTTCTAATTGCTAAAGTTGTGTAAACAGTATTATGGCAGACTCTTCAGTTGTGTGCTGCACTTCACTCAGAC
TGTGAGAAGGTAATATTGGGACAGCAGGTAAAGGCACTCGCTGTGATGTTGCATGGCAGGAGAGAACCAGCTATTTCAGGTTATCCTCTGTCCT
TGGAATATGTGTTGTGGTACATACCCATACACAATTAAATAGGTAAAATGATAGAAGAAAATTACTTTAAAGATCGTTTTCTGCCCAGCAGTGG
TAGTAGTACACGAGGCCAGCCTGGTCTATAAAGCAAGTTTCAGGATAACCAGGGCTATACAAAGAAACCCTGTCTTGGAAAAAACCAAAAACAA
ACAAACAAAAAGTTGTTAAAAAAAAAAAATCCTGCTGTGTGGGAGGCAGAGGGTGACTCAGGCGCAGTTTTCTCCTCCCTCCTTTACATGGGTG
GGTTCTAGGGGGTGAACTAGGGTCCCCAGGCTTGCATAGCAAGGGACTTAACCCACAGAACCTGGAAATCATTCTTTTGATTGAATGCTTTCTT
CATATACAAGAAAAATATGTGACAGTCATCCAGGTAATAGTGCTTAAAAGTAGAAAAATGTATTTACCAGAAACCTAGTGAGGAATAGAACTGA
TGGGCTATTGAGATAACTTTGGGTAAGGTTGGTGGCTGCCATGCCTGAGGAGTTTTTGAAGCAGCTCAGAAAGATCCCCTGTTTATACAACACT
GGTTCAGCATAGTTGTATTCGGTCTCATGATGTAAGAGCTTTAGTGTTTATGTCCTGCTCAACTGCTGCTTGTTGATTAGACTAAATCAGGTAC
CTCAGGACTTTGGTTGCTACTTTTTCTTACGTTAATGCTGGATTGGATGGAAGGTCTTTATAGCCAGAGTTCCTTTCAGTCAAGTAAGGAGTGC
TGAAAGGTGGTGATCTTGACTCTGTTAAACTAGTAAGAGGGTTTCTTCTTTGACCCTTTGTAAGGTAAAAACAAGTCAAACTCATGCGAGACAA
AAATGGCAGAGTATCTTTCTGTGTTTTTATTCATTTGGGATTTTATTCTGCCATATAGGAATGAATATTCCTAAATCTAGTAAGAGGGTTTCTT
CTTGGACCCTTTGTAAGGTAAAAACAAGTCAAACTCATGCGAGACAAAAATGGCAGAGTATCTTTCTGTGTTTTTATTCATTTGGGATTTTATT
CTGCCATATAGGAATGAATATTCCTAAATTTTAGATCTTAGCTTTTGTGTTCTAAAGAGAATGGCTCAGTCTTCTCCTGCAAAAGTCATGCCAG
GTACCAGGGAGGCCCAAGGTGTCTCCCTTGGTGAATCAGGATCCACTCAAGAACCAGAGATGGTGGATATAACCTGTAATACTAGCCCTGGGGA
AGGGGAGGGAGTAAGGTCAGAGTAGGTGCCAAGGGAAGTTTTAGTATATACTGTACTGCTTAGCAAGACACTGTCTTAAGAAAGGCAGGAACGT
GACTGGAGAGATGGCTCAGCACTTTTCCAGAGGCTCTGAGTTCAATTCCCAGCAACCACACAGTGGCTCACAACCATCTATACTGGTGGGATCT
GATGCTCTCTTCTGGCATGCAGCTATACATACAGACAGACACTCAACATACATACCATAAATAAATAAATAAATAAATAAATAAATAAATAAAT
AAATATTTAAAAAAAAAGACAAGCAGGATAAGGAGCATCAACAGAAAATGAGAGTATCATTGTGGTCAGCAATCTTTTAGAAGCCGTCTTGATG
TATACCCTGCTTTTCAAGTGAGCAGCAGCAGCAGCTGGTCTAGGAACATGGAAGCCCTCTTTCAGTGCACAGCTTTACCTTGTAGGCCTTGGTT
GCCCGTGCCCCCCCCCCCCATGTTAAGTACTTAGTAAGTCGACCCTTCTTTTTGTTCTGCCAGCAGCAAAGACACTTAGTTGGAAGGGATTCTG
TCACCATGAGCATATAGTGTGGAACTGTAGAATAGAGAAGTAAGGAACGTGAACAGTTCTGGGGTCATCCAGCTGATTTATTGTAGATGTGTCT
TACTCTAACAGTGATCTCAGGACACAGCAGGCAGAGCTCGGTGTGAAGCCAGCCCAGTCTACAAAGCAAGTTCCAGAACAGCTAGGGCCACACA
GTATAACCCTCTCTTGGGTGGTGGTGGGGGGCAAGGAAAAGAAATTAATATTGGTTAGTTAAGCATTTGGTATTTTTTAACTTTGACTTTTGAC
AAAAAGTTGATCTTGATACACAATCCTAAGAGATCTGGAGCCCACTATATTGCCCGAGAGGTCTTAGACTCAAAAGGAATCTTTCTGCCTCTGC
ATATCCCAAATTCTGAGATTATGGGTATCCTATCATACCTAACTTTATTATTATTATTATAAAATTTGTGTGTGCGTGTGTGTTTTATGTTTCT
GTAGGTATGCTCATGTATCTATGTATATGTTTATGTATGTATTTACATGTTTATGCAGGTATATTCATCTATGTATGTATGGACCCTGATCATT
ATTTTATTAGTTGACCTCACTTCTCAACACAGGGTCTTACTGAACCAGAGCAGTGGTTCTCAACCCGTGGGTCATGACCCTTTTTGGGAGTTGA
ATGACTTTCACAGGGGTGGCCTAAGACCACTGGAAAACAGACATTTATATTATGGATCGCACAGAGGCAAAGTTACAATCATGAGTAGCAACAT
AAATAATTTTATAGTTGGGAGTCACCACCACATGAGGAACTATATTAAAGGGTAGCAGCATTAGGAAGGTTAAAAACCACTAGAGGTTTTCATT
TCAACTAGATTAATTTGTCATTGAACCCAAGAATCCATGTGTATCTGCCCACACCATTTTGAACCTCTTTAATTATTACACAAAATTTTTGAAG
ATTGTGTGGGGAGTGCTGGAAGATGATTCAGTAGTTAAATGCACTTGGTGCCCTTCCAGAGGACCCTAGTTTCATTCTCAATGCCTACATGACA
GCTAACAAACCATATGACTCAGATTCCAGGGAATCTGTATGTGTGTGTGTCAAAAACACTCATATTCTGTGTAATTTGCATGGGGAACTAAGAT
TCTTGCATAAATTATATTGGTATTAAGTTGTTTCACCTTCAATATTATGGTGTTATAATCACATTTATAATTGATTTAATAACCAACATCAGGT
CAGAATTGTAGCCTGTGTGCCCCAGGTACAGTGGTAGATGAGGCCTGCTGTTCTAACACTTAAAGAGTCTGAGGCAGGAGATTTGCTATTAGTT
CGAGGCCAGCATAGGCCACAGAAATAAGTTCAGGCAGGCACGCAGGCATTTCTATGTACAGCTTCCTCTGGGAAGTGAAGTCTTTTTGTGCTGT
TGTTTATTTTGGATTGGGCTTTTGAGACAGGATCTCTGCGGTCTTGGCTGGACTGGACTGGTGCATAGGTCTGGCTGGCCTTGAGTTCGGATCC
TCCCTTTCCTGCTTCCCAGCTGCTGGCTCGGGTTAAAGGCAGGCTCTACACTCTGAGGTATTTTATTTAAGGCATGCTCTACACTCTGAGGTAT
TTTATTTTCTGAGGCAGTGTCTCATGGAGCACAGACTGGCCTTAGACTACTGTGTAGCTGAAGATGACATTGAACTTTTTGCTTCCACATCCTG
AGTGGAATTATAGGTTAGCAACACCTCATGTGGGAATTCATACATTGTAGGTAATCAGTCTACTAGCTGAACTATATCTCCAACCCAGGAGGTC
AGGTTTGTTTGTTTGTTTAACAATCTAGTTTTGAAACAGTCATATCCTAGGCTGGCCTCAAGTTATGTAGTCAAAGATGGCCTTAAAAGATGAC
TCTTGGTTATTTTCCAAGTGCTGGGATTATAGATATGCACACCACCACACCTCATTTGTCTCGGGGCTGGACTCAAATCCAGAGCTTCATGCAT
GTGAGGCAAGCACTGTACCAACTCGACTTTTGCATACTCCATTGAAAGTCATTTTTATAACAGGATCATCTCAGTTGTCTCTGACAATGGGGAT
GTTTTATATGACTGTGTATGGCTTTGCAGCTTTGACAGCCTTATTCAAGCCCATATCTAGAAGGTATTTCTGAGCTTCCTGGGACTCCTCAGCA
TAGAGTCTGGGGAGAAATAGAAGACTTTTGAATGTCCCTTTGTCTTTCGGCTCATGATAACTGCCATAGCTTCAAAGTACTGGTTTTCCAGATT
ACCTCAGTCCTGTTTCTTAAAGAAATTACTCCTGGTTTCTCTGTGTGGTAGCTATTCTGGATGTCTGTGTTTGTTGTTGTTCTTCTTCTTCTTG
ATTTCTTTCTTTTATTATTTTTTAAAGATTTATTTATTTATTATTACATGTAAGTACTGTAGCTACCTTCAGACACCCCAGAAGAGGGCATCGG
ATCTCATTACGGATGGCTGTGAGCCACCATGTGGTTGCTGGGATTTGAACTCAGGACCTCTGGAAGAGCAGTCGGCGTTCTTAACCACTGAGCA
ATCTCTCCAGACCCTGTTCTTGATTTCTGAGAGACTCAGAATCTTTCCATGAGAAGGACCTGTGCTGTTTTAATATTGATTGATTGATTGATTG
ATTGATTGATTGGAGATTGGGTCTCTGTGGTACAGATTGGCATTGAAATTAAGATCTTCCTTCTTGGCCACCTAAGTAGTGGAATGCACCACCT
CTATTGGCATTAATTTTCTTTTTCAGTCCACCACAATATTTTCAGCAGTATATTAGTACAGTTAATAAAAGTGAAGATTGTGTTTTTAATCCAG
TTCTAGATAGAGTTTCAAAAGTTCTGATGAGTGCTGCAGAAAAGGTCTCTGCATTGTGTGATTGTAGCTGTGACGTTTTCAGTTAATCATACCG
TATCTTATGCATTTCTTGCCATGTTTCTGTTGCAGAGCTTGGATTCTGAAGTGTGGAAAGCACTGAGACGTGAAGATGAGTGAACTTGACCAGC
TGCGGCAGGAGGCCGAGCAACTGAAGAACCAAATTAGAGTATGTAGTCATGCGGGTTTGATATGTTTCATATCAGAAACATATCTATTGTGTGT
GGATAACAGAGTGAGATGAAATTGATCTATTATATGCTAGTTATATATCTGAATAACTTATGTTGTTTCAGAAATTTTGTTTTCTTATTAGCTA
CAAGTTGTTGCTTGAATGTACATGGCCCATGTCTGTAATACTTACCAAAGCTTTTGAACGTCTTCCTCCCGGGGAACTAGGTTTTGGACCCTCT
GTGTGGCCACTGTGGCCGCTGTGTCAGTTCTGAAGCAGCTCTGAAGAGCACGGCTACTAAACTGAGAGCAGAAATAGATACTTTAAATGAAGGG
ATCCGTAAAGTGGCATTTTAGATGTCTCTGTCTTATTTTTCATAGTTTCGCAATGGTTTGTGCTCCAGGAAGACCATTGGAATGTATGTAACAG
CTCTTAGGCCACCATAGAGAGCAGACTTGTCTACCTGGGTACAAAACTAAATGGAAGATAATTCCATGTATTCTGAGTTGTTCTTTCTGTGCTG
GGGACTAGTGTTAGAAATGGAGCTAAGAGCCCACACATCCCAGGCAAATGTGAGTTTTTTGGTTTTGGGGTTTGTATTTTAGTTTTGTTACTTG
AGACAGGTTCTTACTATGTAACTTTGTCCTGAAACTCAGTGTAGACCAGACTGGCCTTGAATGCAACATTATACAGAGATAAGTATGTGGGGTT
TTTTGTTTGTTTGTTTGTTTTTTGTTTTTCTTAGACTTCATCTAAGAGCATGGAAAAAATGACTTAAGACTTAACAAGAAAATCCCAGTTTTTC
GTTTGTGTAAGGATTTGTATATTTGAAATAGCTTCCTCTGATTTTCATGTTCTTGTCTGTAAGGATATGGCTGTCTGTCCTTGAGCGTCTCACT
AAGCCTTTAGAATGGTTCACATGTCTTTAACTTGTCAGTTGTGTTCTCAGGCTGCCAGGAGGACTAGGTGGAGAATTGCCAGCATCATTTTAGC
CAACTTTTACTTGAATTTAAGAAAACATACCAGAACTGCATAAGTTTTAGAGCAGAGATAAAACAAAATATCCTCTGCTGTAAGCACCTCACTG
GACATTGGTGTCTACGCTGTTCCTCCGTGTGTCAGTTTCTGTAAAGTTAATAATTTCATGTGTCAGGCAAGTTTATAGCACTGAACTATACCTA
GCCCTTGCCTAAAATCTTTTTAGGGTTAGGATTTTTGGAAGTGGGGCATCAAGTTGGGTTTTACTATGGTACTAAGATGATGTCAAAATTTGAG
TTCAAATGATCCCATGTGTCAGTCTCCCTTGTAGCTGGGATTGTGTAGCATGCCTGACTTTCCCTAAACTTTTGCATGACTTAAAAACTTAAAG
CCAACTTAAAGCCAGGGGCTGAGCATTTTAATCACTGCATTTAGGAGGAAGAAGCAGAGCGATAAGAGTTCAAAGTCATGATCAGCTACATAGT
GAGCTCAAGGCTCACCACGAGACCATCTCTCAAAAGGTAACAACAGACTGGAGAGGTGTCTCAACAATTAACAGCCCTTACTACACTTTCGAAG
GACTCCATTAGGTTCCCAGTGCCCAGATCAAGAGGCTCACAATCTTATGTAACTCTTAACTCCAAGGAATTTGACACCTTCTTCTGGCCTCTGC
AGATACTTTCATATCCCACACATAAACACACACAAAATAAAAGTTTAAAGTCAGGTACAATAGGGCATGTCTGTAATGCTATCAAGTCTGAATA
CTGAAGGAGTATTGAAGGTTTGAGGCCTGCGCATGGGCCATATGCTTTTGAGATTTTTATTTCTTTGTCCCAAAGTTCTGAAAATTAAGAGTAT
AGAAATGACAGAGGAGTTTTCAGACAAGGTCTCACTATGTAGCTGTGGCAGTCCGAGAACTCACTATATAGACCAGTGCTGGAGACTAAAGGTG
ACTAAAGCCACCACGCCTGGCTTTTGTGGTTTGAACTTCCTGGTTTTTCAAGACAGTTTCCCTGTGTGACCCTGTGACTGTCCTGGAACTCACT
TTGTAGACCAGGCTGCCTTCAAACCCATAGAAATACCCTTCCTCTGTCTCCCAATGATGAAATCACACTGCTTGGTGTGATTTCAGTTTTTAAA
ACGGGAGCAGGGTGGAGATGGCTATGCTGTTTTAAATTAAGCCAACATGGAGATCAACTGCACAGCATGGAGGGAAGATTTAGGGAAAGGATAA
GAAAGCCCAAGTGTTGAAGAAAGCATACTTAGAACAGATAGAAAGGAGAAAAAGAAATACTCAGAAAAACTAGCGGATTTACTAAGCCATATGG
TTGTAGCTGTGTAGCTGCTGAGACTCAAATCATTTAGCAGTAGATAAAGAGATTTTTAAAAAAGATTTGTTTGTTTGTTTGTTTATTATATGTG
TGAACACTGTAGCTGTCTTAAGACTCTCAAGAAGAGGGCATCAGATTTTTGTTACAGATGGTTGTGAGTCACCATGTGGTTGCTGGGATTTGAA
CTCAGGACCTTTGGAAGAGCAGTTGGTGCTCTTAACTGCTGAGCCATCTCTCCAGCCCAGATAAAGAGAGATAGTAAATCAAAGATAAATGTCA
TGCAATATTTTCATAAGCAGCTTTACATACTCTCTAGTCCCTAGTACCCACCCGCCTTTCTGCATCCAAATAGTGCCCCTGTAATTATAGCATT
CAAGAGGTAAAAACAGGAGGGTCAGAGTTCAAGGCCAGCTTCAGTGACATATCAAGTTTGAGGCCAACTGGGCAACATAAAGTGTTATGTTTAA
CATATGATGTTATGTTAATATATATTAGTAATATGAAATCTTAAGTTTCATCTAGTCCCATGTAAAGTATTATTTCAGTCAATAGATTCTTACT
GAGATAGTTTACAGTACTTACATTTCAGTTTTGTTTTGGTTTTAAAGTTCAGCTCATGCCTCCCTATAAGGGCATTCTACAGTTCTCTCTCACT
GTGTCTGACATGCACAGATAGTTGGTGACCCCCCCAAGACCTCTGTTGTGGACAGCACCAATTTGACTGTTCCTTATAGGGCCACTTTGTTTCA
ACCTTCATGATGAGCATCATCTTCACAAATGGCTCCATGCGTTTGGCCTTGCCTCTCAGCACTGCACTAATTTTAGAGTCGATTGGTGAGGAGA
TGTGAAGGCCATTGAGTCTCTAAATGGGAAGGGAAGACTTTTGTTTGTTTTTGAGACATGCTCTCACTATGTAGCCTTGGCTGCCCTGCAACTT
GATATGTAGACTACTGGCCTCAAACTGCTTGCCTCTGCCTCCCCAGTTTACTTTGGAGACTAAAGCTGTGTGCTACCTTACCTGGCAAGGAATA
CTCTTTAAAAAAGTAAAATTCTGGAGTTAGATACTTAAGTGGTTAAGTTTAATCCCAGCACTGCTTATAACTGTAGCTCCAGAGAATCCAAGAC
TGCCTGTCTTTTGGTTTCAATAGGTACCTGCATACACATAAATTTAAAAACAAAAACAAAAAACTTTTTTTTCTGATATAAGGGAGGAAAAATG
TAAAATGTTCTTTACTAATTGCCTACTAAATACATGCTTGCTAAAGAACTTAGTACAAGGGCTGGAAGGATGGCATAACTCAGTAGTAGTCTAG
TGCTTTCCTAGCAGTGTGGTCCCCAAAGTTACAGGGAAAATACGAGGATTATCTTCACTTTGAAGGCCAGCCTAAACTACCAATCAAGAACCTG
TCTTAAAATCAGAAGCAGGAATCCCGGCCTGGTGGTGTACGCCTTTAATCCAAGCACTCGGGAGGCAGAGGCAGGAGGATTTTTGAGTTCGAGG
CCAGCCTGGTCTACAAAGTGAGTTCCAGGATTGGGTGAGACGGCGAGCACTTTTAATCTTAGCAGTCAGGAGGCAGAGTTAAGTGGATCTCTGT
GAATTCTAGGCCGGCCTGGTCTACTTAACAAGTTCCAAGATAGAATTATATAAAGACCCTATCTCAAAACAAACACAAAATCAGAAATAGCAGA
TGAGTTAATCAATGAAGTAAAGGTTCTTGCCACCCAACAGTGTAAGCCTGAAAACCTGAGTTCCATTTCTAGAGTCCACGTTAAGGTGGAAGGA
GAGAGTACTGTCTCTACAAAGTTGCCCCATGACCTCCATACTCTGTAGCAAGCACACTGATACACATCAGTCACACTCTTGGGTTTGTTTGTTT
GTTTGGATTTTTACCAACAAAGCGTTTGTGGGTGTTTTGTTCTCCCCTGTCCCCCCACTAACACCAACACAGGGTTTCTCTGTGTATGCCTGGC
TATCTTGGAACTTGCTCTGTAGACCAGACTGACTTCAAACTCAGAGATCCACCTGCCTCTGCCACTTGAGTACGTACTGGAATAAAAGCTGTGT
GCCACCACCACCTATACAGTCAATATACTTTCAATTATTAAAAATCAACAAAAGGCTTCTAGCTGGTGTGTTTATACACATACACAGCTTCTCA
AACTCGGGGGCCGAGGCAGAGGATCAGGAGTTCAAGGTCATCCTTAGCCGCATAGTAACTTTAAGACCAGTTTGCTTACACAGTGAATCACACA
AGAAATGGCAGTTAACTGTAAGAAGTGTTCTCTGGCCCTTTGATTTGTTTTTTATGTGAACTTCCCCTGGGGGAAAAAAGGAAAAACAAAAAAA
GAAGCTTGTCATTCTTTTGTTGATCCCAAGGAAACAGGCTCTCAAGTAGATGTTTAATTAAAGCACACACGCCTTAAATACAGAGGAACTGAAC
TTACTACTCTGATAGACCATTGCACACATGGACCAGAATGTAGCGACCCAGAGCTGAGGGTGGGCCCAGACTTTCCAATTTCACTGTGAGACAA
CAGTGCTGCATGATACGGGCCAGCCTCTTCTGTACTCAAACCAAACAGGACAGAATGATTGATTAAATGGATCAGTCTACATGAATCCATCAAG
AAATCATTGTAAAGCCGGGGGTGGGGGTGGAGTTTTTTTTTTTTTTAAAGATTTATTTATTATTTATTATATGTAAGTACACTGTAGCTGTCTT
CAGACACACCAGAAGAGGGCATCAGATCTCATTATGGATGGTTGTGAGCCACCATGTGGTTGCTGGGATTTGAACTCTGGACCTTTGGAAGAGC
AGTCAGTGCTCTTAACCACTGAGCCATCTCTCCAGCCCGGGGGTGGAGGTTTTAATCCCAGCATTCTGGAGGCAGAGGCAAACAGATCTCTGAG
TTTGGGGCCAGCCTGGGCTACACAGAGAAACCCTGTCTTGAACAAGCAAGAAAGGGAGGGAGGGAGGGAAAGGAAAGGAAGAAAGGAAGGAAAA
AAAAAGAAAAGAAAGAAAGAAAAATTATTGTAGCTGGGCACGGTGGCACAGTTCTTTAATCCTGTCACTTGGGGAGGCAGAGACAGGTCAATAT
GTATAAGGTTGAGGCCATCCTAATCAACATTAAGTTTTAGGCCAGCCAGGTATAAGGGACCCTCTCAATCATAAATCAATCAGTCATTCTCAGG
CCAGCAGGGTGAGTCATCAACTAAAAGTATGTCAATATTTATTTTACTTATAAGTGAAAACACTTTAATTAATATGGTAAAAATTAAAGAAAAA
TTACTATAGTATATTGTTTTGTAGCTCTTAGCTACATTGGTATGTTTTATAAATAGAAAAGATTGCTGTTTTGTTACCAGTCTTATTGAAATTT
TATTAACAATTTTAGATTCCTTGGTCTATTCATAGAACTTAGATTTAATCATTGTTTGGCTTAAAAATAAAGTCCTTGCATAATGCATTCTCAA
AGAGAAGCTGTTGGCCTGAATAGCTAGTTGGTTTTCAGCCTGTTCTTTCATGCAACCCCTTGTGCTCCCATATAACCTGGGAAAGACAGAGCAG
GTGTGTGTTCGCTGTGGGCAGTCAGCCTTGGCTTCCAGGCTGCTCACTGCTGTACCAGCTCCCAGTGTAGTTTCTCCCGGGCCTGCCCAGAGTT
CTATGGGGTCTGCATGTCTTAGTTAGGTTTAGGTGTTGTGAGTTGTGTCTGTTTTGGCCTGAACAACTCAAAAGTGGCAGTGGACTTGGGACTA
TTAGAGAAGAATTAAGGAACAAGACAGTTTGTAGAGGCTTCTTGAAACTTTGGTAAGGAATGTCGTAAAGTTAAAAGTATGGAATAAAACTAAT
TTCCATTTGTTATTTCATAGGATGCTCGTAAAGCGTGTCCCGATGCGACTCTTTCTCAGGTAAGGAATAATTCCACCTTTCAGCTGTCCGTAAT
GCGCAGTCTGCCGCGGGCTTGTATCATGTGGGGTGAGCCCTTTTGTGAGGTGAGGTTGTAGGGCCACTGTCAGGGCTTTTCATTTGGAGGTTGC
TTTGCTGATGGGCAGAGAGGACAGGAACCACTGGTTCCTAAAATGGGAGTGATAGCTCTACCGTTTGACCCAACAATTTAATCAAACTGATTTT
AAAACATGTACCAGGCTGATGAGATGACTGCTACCAGGCTTGACAACCCGAGTTCCATCCCAGAGCTCACAGGGTAGAAGGAGATTCAGAGGTG
GTTCTCTGACCTCTGTGAGCATGTGCCGCTGTCCTGCCCAAGTAAATACAGTTAAACCGTGGGCATCCAGCTCCTGCTGTAGTGTATCTACTGC
TGTGAGAAACACTTCAAACTCTAAGGGAGGAAAGCATTTATATCGGTTTATACTTCTAGGTAATAATATATCATTGGAAAATCAGGGCAGGAAC
CATAGAGGAAAATTGCTTACTGGTTGCTTTCTCAAACAGCCAAAGATAATCTAATCTGTTTAGAAGTTGTCTTGTTCAGTGTAGGCTGGACCCT
CCTACATCTATTAGCAATCCAGACAGTGCTCCATACATAGGCCCACAGAGGAATCTGATCTAGGCAGTGTTTAATTGAAGTTCTCTCTTCTCAG
GTAACTGTGTGTCTAGTTAACAATAAAAACTAGCAACACACACACACCTATACTCACACCTACTCTTGGGTAATCAATGGCCTGATCCTCAGAC
CTAATAGACATAGATTTATTTTCCGTGTGTATCCATGGCAGTGGTAAAGTGAGTATGTTAGGTAAAGGTGGAGATAACTAATAATTTAATAAAG
CAGTAATAAAATGGTAGCTATTCTATGAAATGGTAGTTACATGTGATATCAGATGATCTTAAAATATCTTACTGCATGTACTGGTGCGACAGGG
GATGACAGTGCCTATATCCTTGATAAGAAGAAGCGAGGTACTGAGCCATGTAAAAAGCATAGTGGTTGTGGTGTATTGCTGATAAACATGCTGT
ATACTTAAAGCATGGACTCACTGCACAAGGGAGTTAGGACCCAGGCACTACAGAGTGGATAGTTAATAGTGCTACTTAAGGGAGCAATGTGCAG
CCGGGCAGTGGTGGCGCACTCCTTTAATCCCAGCACTTGGGAGGCAGAGGCAGGCGGATTTCTGAGTTCGACGCCAGCCTGGTCTATAGAGTGA
GTTCCAGGACAGCCAGGGCTATACAGAGAAACCCTGTCTTGTGGGGGAAAAAGGGGGGGGGGGAAGCAATGTGCAATTTAAAGTTTATGAATTG
GCTCCTGGAGAGATGGCTCAGCAGTTTAGACCACTTGCTGCTCTTCTAGAGGACTGGGATTCTCTTTCCAGCACTCTGTAGTGGTTCACAACTG
TCTATAAATCTTCTGGTCTCCTAGGGCTTCTGCATGCTAAAAATATTCTCTCACATAAATATAGGATTTTCCATTTAATATATTTTTGGTTCTT
GGTTGATATTATATTACAGAGCCCACAGAACGCAAACCAAGAGTCTATTTGCTAAAATTAATCTTCACTAATTTTATTTTTTAAGATCACAAAC
AATATTGATCCAGTGGGAAGAATCCAAATGCGGACCAGGAGAACACTGAGGGGGCATCTGGCAAAGATTTATGCCATGCACTGGGGCACAGACT
CAAGGTATGAATGGGGCCTTCCTAAAGCAGTACTTGAAATGTTGCCTATCTGCTGGATTTCTAAAAGGTAATGGTTAGAAAGTTGAATTGGTGT
GGGTGTGATAGCACACTTTGAATCTCTGTACTCAGGAGACAGAAGCAGACAAATCTTTGTGAGTTTAAAGGCCAGCCTGATTTACATATTGAGT
TCCAGGCTAGCCAAGGCTACAATGAAATCCATCTCAAAACCAGTAGGATGGGGTGGGGTAGGAAGTTTGTGGTACCAGTTATCTGTATCCAGGT
CTCATGTTCACAGGTCATACTCAGATTTCCGTTTAGCTGGCTGACTGCACTGCTGTCCCTTGCATAGCAGCACCTGCTTTTCTTCACCCTATAC
CTTTTAGCTGTTGGGTCTCTGTTGTCTTGGCTACTCTGTGGGCTGTGTTTTAAGAGTTGGGCAATGGAAGGTAAAAGGTACTGAGTTTACAACA
GCAGACTGTGGTCCCAAAAACTAAATGTAGACTTCAGTGGTCTCTTCACCCTCACTGGGAAGCCTTATGGAAAATGAACGAATGGCTATCCTTA
TCATCCTTCCACATGGCCCTGCCTGCCATCTACTGGTGTATCATTTAAAGAGATACTACCATTTGGGGGTTCCTTTCTTTTATACTTTGATAAC
TACCCTTTATTGGACTGAGTCTCTCCTGACTTTTCCACTCATAACGAAATAAAGTGCGACATGGTTTGTGTTCTCCTTCCAGACCTAAAGGAAT
CTCAGCATCCTTTTCTTCTGAGTCAGTTTTCATTTCTGCTTAGCTTTCTCTTTGTGAAATTATCTGCTGTAGGGTGTAAATAGCTAGAACAAAA
GATTCTATCTTTAGTTCTCTGCAGGGTCCCATACAATTGCTTGTGCAAATGAGCGCTATCTGAGGCTGTTTAATAATGGAAACAGTCCCATCAA
AAAAGAAAAAGGGGGCGGGGCATTGTGTTCTTGCTGCAGCTGCAGCTTCAGTTAGATAGATCATGGATGTATTCTATTGTTGCTGTCAGAATTG
TTAAGTGCAGATTTGTAGATTTATTGATTGATTGTTTTTTCAGTGCCGGACATTGAACCTAGGGTCTCATGACTGCAAGCAATTGGTGTACCAC
TAAGGTGCACCTCTAGTCCAAGAATAGTTACCATATGGTCCTAAAAATGTGAGATAATGACTGTGTTAATTCACTTAGTTTATATTCTGTAATA
TAAACATTAAACAACTTACAGTCCATAGTTACTGTCAATTAAAAATAAAGATCATAAAAAAAAAACCTAAGCAAAGTTAGAAAATGGACAAACA
ATGGTGGCACTTTTTTTTTTTTTTTTTTTTTTTTTTTTTTTAAACTACAGCCCTTGGAATTAAAGCAATATAGCAAGTTCTAGGACAGCTTGGG
CTACACGAAACCCTGTCTTAAAAAGCAAATAAACCAGGGGCTGGTGAGATGGCTCAGTGGGTAAGAGCACCCGACTGCTCTTCCAAAGGTCCGG
AGTTCAAATCCCAGCAACCACATGGTGGCTCACAACCATCCGTAACGAGATCTGACGCCCTCTTCTGGGGTGTCTGAAGACAGCTACAGTGTAC
TTACATATAATAAATAAATAAATCTTTAAAAAAAGAAAAACAAATAAACCTTGCAAAGCATTATTTAGGGATTGCTTTAAATGAGCATACTTTC
TGGCTAGTCAGTGGTTGCATTTTAAAATCTAAAATAGCTGTTTAAGGAGTTGTTGTGGGTATGCATACCCCTCTACCCCCTTACATCATTTTCA
TATTTTTTGCTCTAATAGTAAAGGGTTGTTATTGAGCTTGGTGTGGTTGCAAATGTCTTTAATCACAGTACTTGGGAGACAGAGGCAGGTAGAT
CTCCATGAGATCAAGGGCAACCTGGTCTACATAGTGGGTTTTAGACCAACCACGTAGCACAAACTTGAAGACCTAGGTTTCGATCTCTAGGACA
CATGTAAAACTGATCATGGCCAGATACTTAGAACCCCACTGCTTCAGTCTGGTCGAGGTAAAAGAATTATTGGGATCACTGGCCCTAGCGGAAA
AATGATGAGCTTCCGGTTCAGTGTCAGACTGCTTGAGAGGAATAAGGCAGAGAGTCTAGAACAGGGAAACAGTCTGCTTCCTTTGACTTCTGAA
TAGGAACACACACACACACACACACACACACACACTAAAATTCAGCTTTGTGGGCGTGTGTGGAGGAGTGGCGGGGGTAGGCATGTGGAGGTCA
GGGCATTATGACCAAGGAATCAGTTCTACTTCTAAGTGGGTTCCTGGGACCAATTGGTTGGTTGGTTGGTTTTTTTGGGGGGGGGGGGGGTCTT
TCAGGAAATGTTTCCCTGTGCATCCTTGGCTATTCAGGAATTGTGTAGATAGGCCTGGCCTCAAACTCAGAGATCCTCCTGCCTCTACCTCCTG
AATGCTGCTGGAATTAAATGGTTGCAAGAGCCCGGGATTAGATTTAATGGAAGGGCTTGTTTGATAGGCACCTTTACCTGAGCCTTCTCTTTAG
CCCTAAAATTTAACTTGTAATACTATTCCAGTAGTGCTTTTCCAGGTCTCTGGGGTTTTTAATCCCCTCTCTACAGAGCCTGATGAAGTAGTCT
CTTTGGCTGGTCTTCCTCTGAGTAAACTGAAGAGTATGTGGGGTTGGTCTATGAGTGCCTCCTTTTGTTCTAGGCAGCTAATTTGGCTAGTTCT
CAGTTTCTATCTCTGTTTGAGGATAGAATATGAAAAGCTTTAGACAATGAACCGTGTTGATTCCTCCAAGTTACATGACCTGAGATTAGAAAGG
CGTGTCTTCTTTCACCTTCTGGAGGATCAGCTAACAGGTTAGCATTGTCTTAATTCCTCACTGAGTTAGCAATTATTGAGCTCTCTGAAGGTGA
CTTCTTAAATCAGAGGATGAGCTACTAAAGATGTCTCTTGATACCCTTGCTCAGAGTCAAAAAGACCAAGCAGAAATAGAGCAGCAGAGGGGCC
TCATACTGTCTGTCCTATAGAAATGGTTGAAAGGACCTTGGGTATAATGACTGAACTGCAATAGATGGGGAGCTGGAAAGACTTTGAGTGACTG
TTAGCCTTGGCTACACAGTACCCTATCTCCAAGGAAAGTAAAAGAATCGTCTGAGGAGTTGGAAATACTAGGTTGTAGTTGAGTTCCCTGTATT
CTCAAGGCTCCAGAGTTTAGTCCCTAGAACCTCATAAAGAAAAAATTTTACCAGAGGACTGAGGTGCAATTAATTGGTGAAGTACAATTTACTG
TGAATTCAATCTCTAGAACTGAAAAAGAAATAAAAGTTGTAGCTGAAAAGAAACTGGATGTCTGTGCCTAGTTCTGTGATAAATTTTGTGACTA
AGGATGGAAAAGGATTTTATTTCTTGTCACATATATATGTTGTTCTTTATAAAGGAAATTTACATATCAGAACATAGCTTGGCAGGGTATGATG
TAGTACAGCTTTAATGCAAGCACTTTGGAGGCAGAGACAAGTAGACCTCTGTGAGTTTGAGGTCATCCCGGTCTACATAATAAGTTCCAGGACA
GTCAAAGCTACATAATAGAGGAACCCTGTCTCAAAAAGGCAAGTGAGTCAGTCATACAAGCATGGTCTGATTAAAAGTGTCAGAGGGCTCAAAA
TGGCGTGGTTGAGTTTTACCTCAAGTCTTTGATGAGCTTCAGAAGTTCAAATATGCCCAAATATACTTGTGGCTGAAATACCTAGAATGTATAT
ATTTTGATTACAATTTGAAGTTAAAACCTGTATGTCACTATCACATGGTAGACCTGGGAGAGCCTCTCACCCTTCCTTAAGAGCACATTGTGTC
TGAGTAATGGTGCTTCCCAGAATTGCTCTCACTCAGTTACTTCCCAGATAACTGAGATTCCTTTGCTTGTTGGTCATGCTTGCCTTATGCAGTA
TATCCTGCCTCAGTCTACAATGCAGAGAACTGCAGGTTCCAAACTAGGACAGAGTCATGAGCAATTCATAATTCACTATAATTGGTGGCCTCTT
TTGCCCAACCATGTGCAATTCTGATGTCATTTATCATCTCACTACCACACAGTTTTATAGAGACAGGTATTCTAATTTATCTCTGTCTGGGAAG
TTTGAGAGTTCGTTTTTCTTTTAATTTTGAGGGATAGAGTCTGCGCCTTAAACTTGTTACATAGTGACCTTGAACTAACACTCACACACACACT
CTCTCCCCCTCTCCCTCTCCCTCCCCTTCCCCCTCCCTTCCTCCCTCTCTCTGTCTGATTTATGGTTTCTCCACATCCCACATTTATGAGTTAC
AACGTATGCTACCATTGGAGCTCATGGTTGTGTTCTAGTTTGATTTCTACTGGTGTGATTAAACACTGAGCAAGAGCAACTTGAAGAAGATTTT
GTTTGAGTTTATAGTTGTAGTCTATCATGAAAGGAAATCAGCAAAGACCGCAGAGGAATACCGCTTACTAGCTTTCTCTTTAGGGCTTACTTAG
CCTGCCATCCAGGACTACATGCCCAAAGACGACACTGCCCACAGTAAACTGGCCATGCTCACATCAATTATTAATCAAGGACATACCCATAGGT
TTACCTATGTGCCAATAAGATGGAGATATTTTCTCAGCTGAGGCTCCTTCTTCCTTAGAGGACTCTAGCTTCTGTCCTATCCCACCCCACCCCC
AATATAAATAAAAACAGGGCAGGTGTTTAAAATGGATTAAAATGGTTCCTGAGTGGGTAGTGTTTCAAGCTTTTCCCATAATATTCAAGCTTTC
TTGCTGTCAGTATGAACAAAACTGTGTCCTAAATATTTCACATGTCTCATGTCTCTGTGTATCTAATTTAACTTGGAATAAAAAAGAAACCCGT
CAGAAGCAGGTCAGATCAGTACCATCTCTCGGGTGATAAGAGAAGAGCTGGGGAGTCTGGTCATCCAGGTGTCCCCAGGGCTGAGTTGTTATCT
GACACTCATGGTATTCCATTGCTCAAGCATTGAGATCCTCTTTCTTATTATTAAGTGTATTATATTTTAAGCGAATGTAGCTTTAAAGCTCTAG
TTGGTCACCAAGATTTTTTCTTTCTGTTTTTTCTTTTCCTTTATCTTTTCTCCAGGCTCCTTGTCAGCGCCTCTCAGGATGGAAAACTCATCAT
CTGGGACAGTTATACCACAAACAAGGTGGGCAGTGCAAGACCCTCCCTGGAACAGTGACAGTCTGTTCATGGAGCAATGATGACCTCGGAGGGT
GATAGAAGAGCTTGTGTTCCTGACCCATTGAGCTTGGCACACAGTTGTGCTCTGGTTCCTGTCTTGGCATGTAGATAGGAGCTGAGTGCTTCCT
TGTCCTTGCACAAGAGCTACACTCCACTCCTAGTTTTGACTGGAAACTTTCCTAATTTATTACTAGAATACTGAAATGCAGATTCCATTTAGTT
AGTAGTTTGTTTTGTAGTAACAAGAGATAATCTAAGATGCACTATACTTTAATGTTTAGTGGAGGATTGGGAATGTAGCTCAGTGGTAGAATAC
TTGTCTACTGGGCATAATTCCCTGGATTTGATTCCCATAACCAGAAAAGATAAAAAACCAATGTAACCCAACAGATTGGTAAAAGTCTGTGATT
CTAGTAACTCAGGAGGCTAAGGTAGGATGATCGAATAAACCCAGAGGGCAAACCTGCCATGGCTAGATAGGGGAAAGGAGAAGACAGAGATACT
AGGTAGAATGTTTGCCTCATAGGTACAAATCTCTGAATTAAATTTCCACTTCTCTTATATTGAAATAAATTACACTGGGTATGGTGGTACATGC
TCATAATCTATCATTTGAGAATGTAAGCAGGACCCTCAACCATTAAAGATTATGCTTACCCACATAATAAAGTGAAACCAGCCTGAACTACATG
AGACCTTGTCTCTAAAAAATAGTATTCACAAAAAACCAAACAAACGGGGCTGGCGGGATGGCTCAGTGGTTAAGAGCACCGACTGCTCTTCTGA
AGGTTCTGAGTTCAAATCCCAGCAACCATATGGTGGCTCACAACCATCCGTAATGAAATCTGATGGCCTCTTCTGGAGTATCTGAGGATAGCTA
CAGTGTACTTACATATAAATAAAAAAAAAAAAATTAAAAAAAAATTTAAAAAAACAAACAAAAAAGAATACATTTAAATTCTGAGTGGTTGCAG
TGGCCAGTCTATAATTCTAGCATTTGGAAGACAGAGACAAGGAATCCACAAAGCAATCTGGTTATCTAGACCTACAGACGAGCTCTAGACAGTC
CTGAGGAACACTGTCTCACAGAAGAAGGTGGAGAGCAGTCAGGGCAGGGCCCGTGTCAGCCTCAGCCTCCGTGTGCACAAGCACACATGCGCAT
AAAGTACGGGGAGCCGAGCCAGGCGTGGTGATACACGACTTTAATCTCAGAAGTTGGGAGGCAGAGGCAGGTAGATCTCTGTGAGTTAGGGGGC
CACCCTGTCTATAAAGTGAGTTCCAAGACAGCCAGAGCTCTAGAAACCCTGCCTGCAAAAACGAACGAAAAGGTGCAGGGCTGGAAAGATCGCT
CAGTCACTCCCTGATTTTTCCAGAACGTCTTGAGTTTTATCCCCAGCGTCTGCGTAGTGGTTTGCAGCCTTCTGTGTGTCAGTTAAAGGGGAAC
CCAGAGCCCTCCCTCCTTTAGCTTTCCAGGCTCTGTACTCAGTTAATCAACAGTTAATCAAATGTTCCTACAGATAACATGAAATTAAATCTCA
AGACAAGTTTTAATGCTGTCATCAATTAAGGAAAAAAAATTGGACAGTTGTATTTTCCCTCAAAATTAGTAAATACATGCCAAGATTACAGAAT
GTTAACTGCTCTATCTAAACAGCATGACTAACTGTGTTACCAATGTGCAGTTAGGTTATTGCATGTGTGTGTCAGAGAGAATGAATATGAATGA
GTTCTGCCTCAGCTTCTTTATTGCTGGGATCGGCATGAATCACCATGCCTGGCTGTTTTTTATGTGCTTGTCTTTGTTTTTTTTTAAGCTCATA
GCCATGATAAATTAAAGGGGAAAGTAAATCTTTTCAAATCCTATTAACATCACTCCTTAAATCATTAAAATGCAAATATAATTTGTGTTTTCTT
TGAGATAGTTTTCCATTATGTATAGCTCAAAATATCCTCAAACTTGCCATCGGGGCTGGTGATATACACCCTACTGGACAAGACTTTTGAGTTC
AAGTCTCAGAACCAAAAAAGACAGACACGACAAAAAGCCCTCTTTTTCTTTCTCTTAGCTTTCCAACTCCTAGATTCACTGACGTATGCCATCA
CACCTATCTACTCATTAGGGTTTTTTGGTTTGGTTTGGTTTGGTTTGGTTTGCTTTGGTTTTGTTTTGTTTTGTTTGTTTTTGAGACAGTTTTA
CTATGGAGCCCTGGCTGGCCTCAAATTCAGAGATTCACCTACCTTTGCCTCCTGAGTGCTGGGGCTACAACATAAACTATACACCCAGCTGTAG
TCATATTTTCTTGAACTTAATAGTGTTTCCTGTCTGTATTCTTGTTATTTAGGTTCATGCCATCCCTCTCCGCTCCTCTTGGGTCATGACCTGC
GCATACGCTCCTTCTGGGAATTATGTGGCCTGTGGTGGCCTGGATAACATCTGCTCCATTTACAACCTGAAAACTCGTGAAGGGAATGTGCGTG
TGAGTCGTGAGCTGGCGCGACACACAGGTACACATCTCATGTTCCAGTGGTGTAAGCTGCTAACTGGGATGTATACTGCTCTTGTGATCTGTTG
ATTATTGGTTCAAGTAAAAGTGAAAGTAACGGCCAGAAATTTTCTGAAAGTGAAGCCAAACCAGGGCCAATATGCCTTCATCTTTAAGAAACAT
GGAAACTGGTTTTTTAGCCAGTTTTCGAATTCCCAGCTGCTTCAGCCATTCTTGTTTATCAACGAATTGATGTTTCAGTCCTGTTTTTAAACTG
CCTTCCATGCTTGGACATTAGCACAGGAATTCTCCTAGTCTAAGCAAGTGTCCATATGCACAGCCATATCTGTTTGTGCTCACTGACCAGTGCT
TTTTTAGAGGAGGCCGAAGAGAGTCTGTGGTCCATTCACCTTTTGAATGTAACCAGCATTAAAATGTTGGGTGCATATTTTTGGAATTCATACA
ACACCATTTAGGACACATTGCCTTTCCAAGGAAGAAAGTCTCATAGTATTTTCTATCTATCCTGTCCCGCAGTGATAGGTAAAACACTCTTCGC
ATGTAAGCAAAGGATTGTTCATAATTAACTGTCATGAAGAACATGGCAGTTTTGTGTTTTAACCCTTAGGGTCAGGTAGGATTGCATCCTTTGG
TGCTTCTTAAGTTACATTGCCAGTGTTTATAACTACTTCAGGAATGCTTGCTTCTTAATATTCTGGCTTTAGCTGGAAGCATAAGCCACCAGTT
TTAGATCTCTTCTTTCTCAAATATATGAACTTAAAAGTGGCCAAATCCAGGCTGGGAGTGGTGGCGCACGCCTTTAATCCCAGCACTCGGGAGG
AGGTAGAGGCAGGAGCATTTCTCAGATCGAGGCCAGCCTCGTCTACAAAGTGAGTTCCAGGATAGCCGAGACTATACAGAGAAGCCCTGTCTTG
AAGGGGAAAGAAAAAAAAAAGGCAAAATCCATACAGGCATGGCTTCCTGTAAATTTTGATCAGTAGTATTTAAATTACTCTTCTAGGGGCTGGC
GATGTAGATTCGTTGGTAGAGTATTTTCCTAGCACACACAGTTCTAGGTCTATCCCAGCACCATAAAACTGGACATCATGATAATTTGTAATCC
CACCTCTCATGAAGTGGAGGTAAGAGGATGGAGAATTCAGTGAGTTTAAGGGCAGTCTGGGCTATATGAAACCCTGTCTTAAAAAAGAAGCATG
GTATAGTTCTCAAGTATATGATAGAAAAAGAAAACAATCGCGGAGAAGTCTCGGTGACTAAGAACAGCCACTGCATAAGCACAAGGACCTGGGC
TCCATCCCAGCGCTCACAAGCAGTCCTGGCGTGTGATAATTTCATCATTGTAGGCTGACTCCAGCCTCGTTCAGTGAGAGGCCCAGTCTCAACG
GACTATAGCAAACAGTGATAGAGCAGGATATTCAATGTCTGGTGTCCTCTGTTAGTCTCTGGATAGGCATACACCACATACACAAATACAAGTA
AGACGGATATCTAAAAATCCTTGGGGGGGGGGGGGTCCTGTTGAATTTGTATTAATTTTTTTAAAGATTTATTTATTTTATGTATGTGAGACAC
TATGGCTGTCTTCAGACATAGCAGAAGAGGGCATCAGATCTCGTTACAGATGGTTGTGAGCCACCATGTGGTTGCTGGGAATTGAACTCAGGAC
CTCTGGAAGAGCAGTCATTGCTCTTAACCGCTGAGCCATCTCTCCATTCCCCCTAAAATATATTATTTAGAGATTTGGAGAGATGGTTAAAAAC
ACTGGCTGCTCTTTCAGATGGCCTGGATTCAGTTCCCAGCACCTGCATGTGCCTTACAGACTTCTATATGTAATTCTAGTCCCAGGGAATTTCA
TGCCTTCTTCTGGCTTCTGTGAACACCAGACATGCTTGTGGAATACAGACACACAGGCAGTCCTGGTTTTATTGCTGTGATGTGACATCATGAC
CACAGCAACTCTTAGAAAGGAAAACATTTCATTGAGGCTAGTTATATAGCTCAGAGGTTTAGTTCATTATTGTCATGGGAGGAAGCATGGCAAC
ATGCAGACAGACATGGTGCTGGACAGGTAGCTGAGTTCTATATTTGGATTCCCAGACAGCAGGAGAGAGTGACACTGGCCTGTCTTGAGCATCT
GAAACATCCACCCACCGTAGCCCTCTTCCTCCAACAAAGCCGTTTCAACAAGGAGGTGTTCCTAATAGCACCATTCCCTATGGGCCCGTGGGGG
CCATTTTCATTCAACACACCACACAGGCAGAAAATTCATATACATTAAAAATTTTGGGGCTGGAGAGATGGCTTAGTGGTTAGGAGCACTGACT
GCTCTTCCAAAGGTCCTGAGTTCAAATCCCAGCAACCACATGGTGGCTCACAACCATAGTAACAAGATCTGACATCATCTTCTGGAGTGTCTGA
AGACAGCTACAGTGTACTTACATATAATAAATAAATAAATCTTTAAAAAAATTTTTTTTTGAGAAATATTTAACTTACTAGTTTTTAGCTAATT
TTAAATTTGAACTGGTCGTCTACCAAGACATCATGCTATAAGAAACAGAAACTGTATTATCTGGTTGTTGGGCATAGTGTGTGTCTGTCTTTCC
ATCAGAAGGAAAGAATCTACATCTGTGTATTTCAGGTGTTGCATGTACATTTATTGCTGCTGTCTGTATCTCCTTGAATGATCGGTTGTTTTAT
CATGGCTTAGTATCCCCTCTCATATAATACTTTACTGTGAATCTACTTCATGTGGGGTTTTCAGCCAGCTCAGCCTCAATGCTCACTGTCTGCA
TGACATTTTTTCTTGTTGATTGTTTTTCTAATAAGTTCTCTCATACACAGCATAGAGTTAAGTCTTCACTTCTGTTTAAAGTCTCTGCCTTTAG
GAGCTGGGAACGTAGCTCCTTTAGGAGCTGGGAAAATAGAGTGCTTACCTACCATGAATGAAGCTCTAGGTTTGATCTTCAGCCCAGCATAAAA
CCAGATCTGGTGATGTAGCCCTGTAGTTCAAGCACTTAGATAGATGGAAGCTGGGAGTTGAAGGTAATCCTTTACTGTGTAGTGAGTTTGAAAC
TGCCTGAAGTACACGACAGGTAGCTTTTAAAATAAATGGTTGGGGCAAGTCCTGTACCTTGTAAATACTTTACATATAGTTTCTTCTTGACTTA
TAATGAGGTTATATCCAGAAATATCAATCAATCAATCAATCAGTCTCTCTGTCTCTCTGTCTCTCTGTCTGTCTGTCTATCTGTCTATCTATCT
ATCTATCTATCTATCTATCTATCTATCTATCTATCTATCTATCTATCTATCTATCTAGGGTTTCTCTATATAGCCCTGGCTATCCTAGAACTCA
CTCTGTAAACCAGGCTGGCCTTGAATTCAGAAATCTGCCTCCCAAGTGCTGGGTATCCAGAAATCTCTTTAATGTTTAAAATATTAGCCACTGA
ATTATGTTGACTCTGTCTAGCCTGCCATCCCACATCTTCCTCATCTTATATTGACCAGGCTGGCCTGAAATCTGCCTGCCTCTGCTTCCTGGGT
GCTCAGATTGAAGTGTGTGCTGCTTCGATGGGTGTATAAACTCTTTGTAGCCTAGGACATTTGAGGCTGCTGTGTTACATTCTTACAGTCCTGA
CAGAGAGCTTCAGTTCATTGCTGATAACCAGTACCCTGGGCAGATTATACATACCACTTATTGATAGGTTGAAAGAATTGCATTTAAAATTTGA
AGTTCATTTGCACTAAAAAAATCCTAAGTGTTGCTTTGGATGTGGTTGGAGTTGGTACTTAGGGTGGTTCTCTCTCCCTCTGCTTTCTTTTCTG
CTGTCTGTCTTTCTTTCTTTCTTTCTTTCTTTCTTTCTTTCTTTCTTTCTTTCTTTCTTTCTTTCTTTCTCTCTTTCTTTCTTTCTTTCCTCCT
TTCCCTCCCTCCCTCCCTCCCTCCCTCTCCCTCTCCCTCCCTCCTTCTCTCTCTTTCTCTCTCTCTCTCTCTCTCTCTCTCTTTCTTTCTTTCT
TTCTTTCTTTCTTCTTTTGGGTTACTCTGTTTCTAGCTCTAGCTTGCTCCAGCCTCTGCATTCTTTTCCCTCTGCCTCCCCAGTGCTGTAATTA
CAAGTGTGCACCACCATGCCTGGATTCAACATTGTGTTTGAAGAGTTGTATAGGCTCACATGCACTCAGACAAACAACGTCCTCATTTCCAGTG
CTGTGTACTCCTTCCTGGGGGGGCATCTCTTTTTCTGGTTTCCTGTATGCCAAAAAGCTACTCCCTCATAACACCTACAGTTGTACATTTTGCC
AGTCCCTTTATGTGCTATTTTTTTTAATGTCTGTATGTTTGTTCAATGAAATTTAATTTACAAATTAAAGAAAAGAGATTAACAGTAAGCAGTA
AGAAGGTACTGCAATATAGCAATACACTATAATTAAGTTTTTGTTTTTTTTTTTTTTGACTCATGGCACTGAGAATTTTATTTATTTATTTATT
TATTTATTTATTATTTTTTAAAGATTTATTTATTTATTATATGTAAGTACACTGTAGCTGTCTTCAGACACTCCAGAAGAGGGCATCAGATTTC
GTTACGGATGGTTGTGAGCCACCATGTCGTTGCTGGGATTTGAGCTCATGACCTTCGGAAGAGCAGTTGGTGCTCTTAACCACCGAGCCATCTC
ACCAGACCTATTTTTTATTTTTTAAGGAATTATTTGTTTCTGGAATTTGCTTGGTTTCCATGGGTTAACTGAAACCAGATGAGGCTGCAGTGGT
TAGAGTAAACTGCTTGTGAGAAGTCTTGATTTTTTTTTTTTTTTTTTTTTTTGAAAAGATCTGTTTTATAGTAGTAGTAGTAGTTGTTGTTGTT
GTTGACAGGGTTTCTCTGTGTAGCCCTGACTATCCTGGAACTGGCTCTATAGATCAAGTTGACCTCAAACTCACAGAGATCCATCTGTCTTTGT
CACCACCTTTGAGTTAAATTATTTTTATTGCTTTTATTTATGTGGTGGGGGTCAAACATGAGGAGATCGAGGATGATTCTATAGAGTTAGTTCT
CTCCTCTCATCTCTGCATGGGGTCTGAAGATCAAACTCAGTAGGAAGTTCCTTTACCCACTGATTCATCATGCCAGTCTTTGACTTTCATGTGT
GTGTGTGTGTGTGTGTGTGTGTGTGTAATTAAGAGGACAACTTGTGGGAAGTTGGCTTTTCTCTTCCACCATGTAGGTCCTGCAAATTGAACTC
AAGTAATCAAATGGCAGCAAGCACCTGTACACATGGTCCCATCTTGCTGGCCCTTGACCTTTATTCCTGAAGGATAGTTTTGCTGGACATACAG
GATTGATGTCACCTTACATACAGTTGGTGTCACCTTCATGTGTATGTGTGTTTCCCCTGCATAAATGTTGATGCACCACATGCATGGCTGATGC
CAGCGAAGCCAGAGTGAGGGTACTGGGTTTCTCCTGGAACTGGAGTTAATAGTTACAACCCATCGTGTTGGTGCAATGAATTGAATCTGAGTCT
TCTAGAAAAGTAGCCACTGTTCTTAACCACTGAGCCATCTTCTCAGCTTTCAACATTTAAAAATATAACATATTAAAGGTGTATCTTTGATTTT
TGCAGTTTGATGGTGATATTCCTAGACGTGTAACTTTCTTTATATTCTTGTGTTTCGCATTTGGAACTCACTAAAATATGTCTTTTGCTAAATC
GTAAGGTTTGAGCTGTGTCTTCCAATGCCCTCGTTTCTATGGTTTTGGGGTGAGCTGCTTCAGTTTCCACTTCCTCACGTGTTTTGTTTCTATT
CAAGTCAATTCAGTTGACTTTGGTCACTAGACAACAGCTTTCCTTCTTGTTAGACCAGCAGTGAGCAGCATTTGAAGGAAGCTCAGCTGGAGTA
GACGGCAAGGGCACAGACTGGGCTGGGGAGGCATGGAGAGTGCTCACATAAGTGTCCACATTTGTGTCACATGGTCTCTTCAGATTCAGAATTT
CTGTGTTTTTATTTTAAAAGATCCCCTCTACAAGCACTGAGGCTTTATGTCTTCTTATTTATAAACAAGATTATTTCTCATGTTTCTGAATATA
ATTACAAGACCTAAATGTAAAATGTCAGCTAATTGTAGCATCTGATCTTGAGGTATATCTTTGTGGTCCTTTCCTCTTGAGTGTGGATCACACT
TTGCCATTTCTTAGTGCACTTGCAGCCTTGGCAATATAATTCTCATAGCAACCGTGGGCTCGTTATGTTCCTTTCAGATGTCTGTTCACTTTCA
TCAGGTTAAGAAACACATACATAGCAGCAAAACTCTTACTTGTCCTCCTGTCACTGGAGCTGTCTGTGGGTTTGCTTGCTGCCTGTGTGCTGTT
GCTGTTAACTATTAAAGTGCCTGTCTGTGTAAGTTAGGACTGTTGGACGCTGCTTTAGCACTTCTCAGGGATGTTCCAGCAGACTGGCTGATCA
TCCAACTGTGTAAGACAAAACCATTGAAGTCCGTTGAAGAACAGGCGGTTGTCCATTTACGAGTTTCCTATGCCCTCCAGGGTCAAAACTTAAT
AGAATGTATAGGCTATCTTATAGGATAAGGAAACCCTAGAAATGGATGTAATTGGGTTCACAATAACTGAAAGAGTGTTCATTATTACTATTTA
TATTATTATTTATAATTTATAGGTAGAATGTATATTCTTTTTTTTTTTAATTCAAAATAATTAGCCAGGTGCTGTGGCATACTCCTTTAGTTCA
GCACTCAGAAGGCAGAAGCACAGCACTCTGACTTCAAGGCCAGCCTCATGTATAGACTTGAGTTCCAGAACAGCCAGGGCTACAAAGAGAAACC
CTGTCATAAACAAACAAACAAATAAGACAGGCACATACATATGTACATGTGTGTGTGCGCATGCGTGTACGTGCACTTTTTTTCTTTTTTTTTT
TTTTGAAAATGTGACTGTTGCCTAGATACTATTTTTAAGGCGACTGGAACGTAGGCACAGATTGGTCTCAATGCTAATCCCTAGGGACTGATGA
AGAAAGAGTGGAAAGGTGGCCTCACAGCTGCTGTTTTGAGACCTTTAGTTCTGGTCCCGAGGGCCATGATTTGCCTGCCCTTCTGATGTAACAT
GGCGTTGCCATTTAGTATTGTGACCTCGTAGAGTGAGTAGCTAGAACATTCTGGTGTATAATTTTTTTCATCTAATGAATAATTGGGTGTTTTA
CATATTTATGAATACAGTGGGTTGTCTTTGGGTTTTTTTTTTGTTTTTTGGGGGGCTGTTTGTTGGTTGGTCTGGGTTTTTTTCTCCCCTCCCT
CTGAAGAGTATTGGTGACCTTGGATATAGAAAAAAAGATTCTGTCTGTCTGCAAAGTCAGCTGTGAAACACAGAGCTGGTCAGTTACTTACTTC
TGTTACTGTCGTCTTGGTGTCCCCTCCCTCCATGTCACTCCAGCAAAAAGGAAGTGCTACTTTCAGGGCTAACCACACAGGCAATTGCAGCACG
TACCTGTGCACAGAACCCATACATATCAGGGAAGTTCTCAGTGTCCTGCCTTGTTGAGTGAAATTTTTTTTTAAAGATTTATTTATTTATTATA
TGTAAAGTACACTGTAGCTGTCTTGAGCTAAGACAGGATCACTGCTACAGGATCACTGTAGCTCTCTTCAGACGAACCAGAAGAGGGCGGGTGG
TCGTGAGCCACCATGTGGTTGCTGGGATTTGAACTTCGGACCTTCAGAAGAGCAGTCTGGTGCTCTTACCCACTGAGCCATCTCACCAGCCCCC
ATTGTCTGAAATTTTATCTAACCATTTATAATTTGTCCCTTGGCTTGGCAGATTTTACTCCACTCTGGCAGAACTATGTGACTTTCAGATATCA
GTCTTTTCAGTTACTCATTTTCTTCTCCCTCAAAATTTTTATTGTATTTTGAGGCAGTCTCGCTGACCCCTGTTCCATCCACAGCTTCTAAGAA
ATCTTTCCAGTGGGTGTCAGCAGAAACATAGCTATTCTGTGCATACTCCATATCACAGCTCTTGAGAAAGCAAGCGCAGTCACCCGTATTTTTG
GAGCCTTCCCACTGGATACTTTCAGTGTGCTTGCTGTTCCTCTAGTTGGCCTGTAGCCCTTTCTCCCTTGGTTCCGTCACTATCTCCCTTCTCA
TTGGTTCTGAGCTCCTTTGTGTCCAGCTGCTCTACTCAATAAGTAGATGAGCACACAAATGAGGATAAAAGGAAATGGAAAGACACAGGTGGAA
GGATGAGGTGTTCCCAAGGCATAGCTCACTGCTATGTGCTGTGCAGGTGTGGGTCCGTGTGAATCATGAACCCATTGAGTCTGCCTATAGTTTC
ACCCATCCCGTGACAGACCTTCTGCGCATTTTTATTAACGTGGTTTTCACAGGGAGAGGCTCCTAAGATTGAGACTTTCTAGTCTTGGTTATTT
ATTTGTGTCTGCTCTTTTTCAGGTTATCTGTCCTGTTGCCGGTTCCTGGATGACAATCAGATAGTTACCAGTTCTGGAGACACCACATGGTGAG
TACCTGACAAGGCAGCAATTGAGGCTTGAGGTCTGTCTACTGTTCTTGTTAATACTGGTTAGTAGGGTTACTTCCCATTGCAAGATAAATGCTT
CTTGATCATAACCTGACAATTAGAAAACAAAGTATCAATGAGTAACTCATTGCATCATCTGTGGATATGCAAAGTTTGATTTTGAACAAAGAAC
TAAAATGTAGATATTTAAACTATAGTTGTATATCAAGTACCTGGTTTTTTCTCCATTTATTTTTACTCATGTATGTGGTGTTTTTAGCCCTGTG
GGGTTCTTTTTCACTGTAAAAGTCAACTTAGCATATTGTGAGAATGAGTAAAGCTTTCAAACAGCAGCTCTGTCAGATTTGTTTATTTTAGAAT
GTTTGCTTACTAGTAGATGGAGACACATTCAAGTAATTTTTCCCTTTGTTTATTAATGAAATGACCAGCTGGTTATTTTAAGAGAATCATTTTA
AACTTAAGCTGCCCTTGTGGGTTTTTCTGGTCGTGGGAGACATTCCTGGGAGGTCAGTAGCAATATTTTAAATTTGATTTAGTAGCATGGCTTA
CAGGCTGGTGAAATCCAACAATGGACTATTGATGAAAATGTTAGCCAGGCGTGGTGGCGCACGCCTTTAATCCCAGCACTCGGGAGGCAGAGGC
AGGCAGATTTCTGAGTTTGAGGCCAGCCTGGTCTACAGAGTGAGTTCCAGGACAGCCAGGGCTACACAGAGAAACCCTGTCTCTAAAAACAAAC
AAAAAAACAAAAACAAAAACAAAAATGTAGTCAGGTGGACAGTGAAGATTAGTCATTACAAATTTCAAATTGCATGTGTTGATTATTTTCTTGG
TTGAAATATGTGAAACTTTTGTGCTCTGTGCTTCTTCCTTCCAGTGTCCCTCATGCCTAGCAAAGATAACCAGCATTCATTGTAGTGAACTTGG
TTGATTGGATTGGTGTTGCTGGGTGACACAAGCTGGTGAGGTTGACAAGAATGGACAGTGTATCATGCTGTTGCTTCTGCGGGTTGCAGCAGGA
AGTAGCCTGACTGCCATTTCCATAGCCAGCTCCCGCTTTATGTAATGCTGATGATATCGTGGCTTTTAGCCACTCTGTCAAGCAGAGGCCCTTG
GAAAACCTTGTACATCCATGAAGACCATCTTTGGTTTATGGTCCTCGTTGCAAGAGGAGTTGGAGCATCAGTTTCTCTGAGCCACTTATTTACA
TTCCTAGTGTCTAGGAAGGTGGAAAATGAGGGGATGGAAAGAATAACACAGGTGAAACAGGCTCCTTTCTTTCAGACCTTAGCATTTTCTGCCA
TCCCACAAACTGCCCATGATTATCTTGTGTGAAGGACAGTGGCTCCTTTGCTGTAGTGTGAGGTAACCTGGAAACAGCAGGCCCATATGCTCAT
CCATATGGTTGTTTATTGTTTTCATTTTCTTTTCCTTAGCAAAATCTCATCACAGAAGTAAATGTGTCTTCCCCGTGGCACAGAAGTCATCTGT
GATTCTGGGTAGTATAGAGGATGTTGTTCTAGGTGATTGGTCCCATGTGCGTGCCTGATACCTACTTTTCATTTCTTCCTGCACCAGTGCCCTG
TGGGACATCGAGACTGGCCAGCAGACAACCACATTTACTGGACACACTGGAGATGTCATGAGCCTGTCTCTTGCTCCTGACACCAGACTGTTTG
TCTCTGGTGCTTGTCATGCTTCAGCCAAGCTCTGGGATGTCCGAGAAGGGATGTGCCGGCAGACCTTTACAGGACACGAGTCTGACATCAATGC
CATATGTGTGAGTTTAAATTGTCTCCTTTAGATCTCAACCTTTGTTGTTGTTGTTGAAGATTTGGGTTTTTTTTTAGGGTCTGGAGAGATAGCA
TATTAGTTACTTTTCTGTTGCCGCAACAAAAACACCATGATCAAGGCAACCTAAAGAGAAAAAACATTTCATTGAGGCTTACAGTTTCAGAGGG
CGAATCCATGTCCATCACTGTGAGGAGCCTGGCTGCAAGCAGGCAAGCATGGTGCTAAAGAAGTAACTGAGAGCTTCATCTTGAGATATAAATA
ATCACAAGGCAGAGAGTGAGTGAGCGAAAACTTGGAATGGCATGGCCTTTTGAAACTTCAGGCATCCAACAAGGCCATACTTTTTAATACCTCC
CAAACAGTTCCACGAACTAGGAAATGAAGTGTTCAAACTTGAGAGCATGTGGGATCCATTCTCATTTATATTTGTGTCTGCCTGTCTGTGTATC
TGTATCTGGGTGTGTGCATGCCTATTAGAGCCAAGTACGCTGTTAGCAGACACCTGATATAGATTGGGTGAACCAAACTTGGTTGTAGTAAATG
GCTTTTAAAGTACCTTTAAAGTACCACTACCTTTAAAAACATTTTTTTTCAAAGTTTGTTTTAAAATTTGTGCTCTGGTTTTTTGTTTGTTTGT
TTTGTTTTTTCTTTGTGTTTATTCTGAGAAAAGGTCTAGTAGTATAACCTAGGCTTACCTAGAACTGGAAGTCTCTCTAGCACTAAGATCACAG
GCAGGCCTTTCTACCAAACCAGGTTGTCCTTTTCTTTGCAAATACAACCAGAGTCAGAGTGTTTTAGAAGCAGCTAAAACCCTGTGCTGTGCAT
ATATTTCAGACACTGATGAAGCCATTAGGTGCCAGCTCAACTCTTCCTTTTTTTTAAGTGACCAGTAATTTAGAGGCTGAATATCTTGCCAGCT
AAGGGAATGGCAAATTTAGAACTTACTTTCTTGCTAGCACCTCCTGAAGAGATGAGTCTGGTGACTAGAGCAGTGACTTGCTCCATCCTCCACA
AGTCCCTCCTGACAGCTCGTGTCCAGAGGACAGAAGCCCTTCAGCAGTGCCTCAGTCTCATTGCTTTTCTGTACTGAAGTCTGCCTTGTTGACA
GTGGCAGTCCATGAGTACTATGTCAGTACCCCCCTAGTACATCCCCCATTCACTCTAGGATGACAGCCCTTATTCTTGCTGGAACATCATCCTG
TTTTCCTTTCCATGGCAGCCATGCCTCTGCATTAACATCATGTCTTGTTGAACATTTACTTTTATGTTAGTATATATAATTCAGCTTGACCTTG
AATTCACTATGTGCCTCAAGGCTGTGTACTCTATTCCATGGTTGATTACAGATTACTACACTTTTTAGCCTCCTGTGCTTGTGCCTGAACTATG
GGCCTTACCCACGCCAGGCAAGTGTTGAATCAGCTGGCCTGTGCCCCAGGCCTCATTGTTTACATATGTCTTGATTTGTCTTCTCTTTTTAGTT
CTTTCCCAATGGCAATGCCTTTGCCACTGGCTCAGACGATGCCACATGCAGGCTGTTTGACCTCCGTGCAGACCAGGAGCTCATGACCTACTCC
CATGACAACATTATCTGTGGTATCACATCTGTTTCCTTCTCCAAGAGTGGCCGCCTCCTCCTTGCTGGGTATGATGACTTCAACTGTAATGTCT
GGGATGCACTCAAAGCTGACAGAGCAGGTCAGTGGCCACATGGCCTTTCTGTGTTTTGGGGGCACCAGCTTGCTTCTTTCTATGTGGTAAAATG
AGGTTCAGATGAGCTGAAATGTGTACATTTGTATCATCAGCTTAGTPATGTCATAGCTCAGCAGAGGGGACAGTTAAAGGCCACATAATTTGAG
CTCTGGCACTCCAGGAACTACCAAAAGCTCTCCTGTGAGGACAATGTCGGAGTTGATAGTATGCCAGATACCACTGAGTTCAATGTTCAAGTTT
CCCATTTTGTCCTGTGGGTATCCATGGTTAGCCTAAAACACTCTATTAGACCAGATGTCCTTGAATTTAAAAGTCTTCTGATTCAGCCTTCTAA
ATACTAGAATTACTGGTGAGTACCATCAATTTTCAATCTTTCTGACTCATTCTCTGTAGTCACTTAGCTTCTGACATATTCCACCATGGACAGC
TCCATCTTCAGCTGTCTGAGCCTATCTATACAGCTTTGGCATCTGATGCCATGCCCCTTCGTCTGTACAAGGTTGTTGAACACTGTGCTGGTTG
CTGGTAATGATTCAATTTAAAGCCTCAATTTAATATGTCAGAATCAAGTGGATTAAATACAAGCTGGACAAAAGATTAGAAAGTTTTTGTCTCA
AGAGTCTCCCTTTGAGCTCAAGCTGGCCTTAAGCTTCAAGTTGAAGAGCTTTATTACATTCAACAAATCTGCTTTGGAGGAGGGAGGGAGGGTT
GTGACCATGAATCTCGTGGGTCCCACTGTGGCTGCTTATACCCACCAGACTACTGAATTGAGGACAGTTTTTGGTGGCTCACACATTTAATCCC
AAGTCTCAGAAGCATGCAAATTCTGTGAGTTGTAGGATAACCAGGGCCTGTCTCAAAAATAAAAATCAACATTTCTGCCTTTGCCATTGTAGCC
CAGGAAGTTGACTCTGAGTTTAAGAGAACCTTCCAGCCCTTGTTCTTAGTTGGTACATCAATAGCTACAGGACTAAATGTTTCTGAAGTTTCAA
ACCCTTCTGTGAGAGAAGGGGTGTTTTAGCAGGTGATGTGCCATGTGACAATGGGTCCATGATTTCAACTGTAGGCTCTGTGTACCCTGTGGTT
AGTTGTTCTGAGTTTACTATTCAGGCTTTTGGTTTTTTGGGTCATTATGTTTTGTTTTGTTTTTTTTAGAGATTTATTTATTGTAGCTGGACAC
TGGTGGGTTATACTTTAAATCCCAGCACTTGGGGAGGCACAAGCACGCAGATCTGAGTTTGAGGCCAGCCAGGTTTACAGTGAGTTCCAGGACA
GTCAGAGCTACATAGACAAATCTCTCTGGTGGATGGGGTGGATTTATTTATTTTATCAATATGAGTGTTTTGCCTGTGTACCTGCAGAGGCTGG
AAGAGGGTATTGCATTCCTCTGGAACTAGAGTTACAGATCTTTGTGAGACGCCATATGGGTGCTGGAAACCAAGCTCAGATCCTGAGACACAGA
TGGATGTTTATTCATTATTTACTTGTTTGTTCATTTGTATTTTCTAGACAAGGTTTCTCTGTAGCCTTGGTTAGCCTGGATCACCAGATCTGTC
TGCCTCTGTCTCCTCAGTGCTCGGATTAATCCAGCATGTGCGATTAAAAGGCATGCGCCACCACTACCCATTTTTGTATACTATCAGCTTTTTG
TTGGTGTACAAAAATTATCCCAAGGGGGTCTGTGTTTATGTCCTTGTTCTTCTTAGAGAAGAACATGTTATGATTGGTTGCTCTCTTAACAGAA
TTGCCCATACACCTAAGGATCGTTTCTACTTGGATAAGAACCTTCTCCCTTTGTAGTCGCCTTCCCTTGCCCTCTTAAGTGCTTGTTTTACCAA
AGTCTTGTAGCGTTCTTCATCCAGATGAGCCTCCTGCTTTGTCACAGCCTCATGTGTGTTTGTGGTTGTGCTCTTTTTGTTCTCAGGTGTCTTA
GCTGGACACGACAACCGAGTCAGCTCCTTGGGGGTGACTGATGATGGCATGGCTGTGGCAACAGGGTCCTGGGACAGCTTCCTCAAGATCTGGA
ACTAAAGCCAGGAGGTAATGGGGCAGTCCCATTCCCAGGACATGGCATTTTCCTATATGGGTTATAGATTTACACAGATCTTGTTGATCTGGCA
TTGTAGCAGATCTGCAGTGAAGGCCCTTAGCTGTGATGCAGAGAACAGTGGGAAGAGCTCGGGTTCTTTCTGTGTGCACTGACTCCAGGCTCTG
TTCACAGCAGCAGGTGGATGGATGCCATGGCGACTGGAAGACCATTCCAACCTTGAATCGTTACAGCGATAGCATATCCTATCCAACCATACTA
ACGTGGACACCCACACCTCCCCTCAGAACTTCAAAAGGGCAAGATCTTTTTTCCTTCACTTATTGCTGAAACCAAGAGCACAATTCCCATTAAG
AGAAGGATCTCTGTGCTGTAAACTAAACAAATTGTGCATTTTTTCTGGGGCCATTGTTTTTGGTTTATTTTGTTATTTTGTTTTGTTTTTGTTT
TTTTGGTTTCATTTTGTTTTGGGTTGGTCCAATTTTAAAAGGAAATACTACAATAAAAATGTTAACCAGAAGGTAACCCTGAGTGTGATTGTGA
GAGAGACACGCCTTTTTACTAGTTCATTTGCATTTAAATCAGTGATCCTGTTCTGTGGCATTCATGAAAAACAAGTTGAAGAATTTGTTTATCT
GGAATTGTTTCTAAATATAAGTCATTTGTAGCAAGACTTTGGAAGCATTCTTTCTTAAACATATTCCTTTATGTTCAGTAGTTAAGAGAACCAG
GGCCCACGGCGGTCTCTGACTTGTCACTGATGTTTTTTTTTTTTTTTGTCCCTGGCTTGCTTTTTTTTTTTTTTTTTTTGTCTCATGTCACAGA
TGACTGTTTCGCACAAACACAGCATAGTGGAAGAAAAAATTTCAGAAGCAAAGTAGCCAAGCACATGTTAGGATGGCCACGAATGCTTGGTCAG
AGTCCTTCCTTCCCTTGCTCAGCAGCCCACTTACTCCCAGCGCCTTCCCTCCCTCCTGTCCCTCCTGTCCCCTCACTGTCCACTTTCTGTTCTT
TCCTTGGGTTTCCTGTGAAGTTGGAGGGACGTTTGTAGTCATGTCACACTAGCCGTCCTGCCCCTGCTGTCAGAGATTTTTTTCTCCAGAGAGG
TGCCTGTCCTGTGCTTGGATAGTCAGTTGGTTATTTGTGTATGAAACAATGTACAAATCAATGTTTTGAAAATAATGATCTCAGACTTTCTAAG
TTAAATTTTAAACAATTTGATTGTTTGCCATATTGGGTGGGGTTACTCTTAGAATCGCATGCTGTAGAAATGCTCTAAAGTGCATATAGGACTC
AGTCCTTAGATGTTCTTTTTATTTTAAGAAATAACCTCTTCAAGTTGTAACCGTGGCGGCTTTGTCCATTACTGTTGCTATTCTGCACACATAT
CGGAAGCAGCAAGCGCTTTACACACTTGAGTAGTGGGATAAGTCACTGTTTTCTTTACTTTACAAAACAAAACAGTCCTGTTGGGAATGATTGC
TGTTGGATTCTGGAAGGGAGGGGAAAGCTGCTCTGCTGCCTCTTTGCCACTTCCTGGAAGCCTTCCTCCCTGGAAGAACAGCCTTTTGCATCTG
AGGCGACCATTTTTACACCATGCAGATCTTCGTGGTTATCATCTCAAAGCCATGTTCAGCAGCAGCGTTTGGACAGCAGTGTTCTGTGGGCCCT
CCCCTCCCCCATATCCTCCACCCCCTCATAACAGTAGTGGCTTCGCCATTCTGTTTTCTGCAGTGTCCTGTACAAAACTGTGCTGTGACTGTGC
GGTAGGCCTGGACATGGCAAAGAGAATACAGCGAAGCCCCTTCTCTTCCTACCAAACAACTCTGTAGAGCTCTCTGCACCCTGTACCCTTTTCA
CCTTTTGTATTTAATTTTAAAGTCAGTGTACTGCAAGGAGGCTGGATGCAAGATAGATACTATATTAAACTGTACTGTTATTTAAGATGTAATA
AAGCAGTTTGACATGAGGGACTCTGGTGTGATGGTTATTTGAGGGTCATCTTCAGTGTGTGGGCGTTTGCCCCAACTGCTGACTCAACAGTTAT
ATAACGTCATGAGAGACCCTTATAAAAGTGTCAGCTCGGGCTGAATGTAGTAGTACACACCTTTAATCACAACACTCAGGAGGCAGGGCCAGAT
GGATCTCTGAGGTTAGGGCCAGCCTGGTCTACTTTGCAAATTTTAGGACTGCCAAGCAATATAGACACTGTCTCAAGGGACGTGGGGAGATGGG
CTCCTCGGAGGTTTCTGGTATATATCTCAGGTCTGCAGAGCCACATGTTTATCACGACAAGTGGCCTTTCACCCTCAGCACCTGGGCCCTGTCT
GCCGTCTCCTGCTGGACCAGCCAGCTGCTTCGACTTCCTCTTCCTGCACTAAAATGTCTGAGGACCTGCCCCTCCAGAGATACTTTCCTGTTGA
CTAAGCACCATTTCCTTCTCTGTCGATAAACGTCACTGCATCTTGAAGGAGCGACTATCAACCCCTGAGTTGTTGGACTGCCATTCCAGCTTCT
GACACCCATTCCACTGCAGTGCCTGCTTCTAAGTTTTATGATAAAAATTTTAATCTTAAGGTTTTTTTTGTTTGTTTGTGGTGAGTTTTTTTGG
TTTTTTTCAAGACAGAGTTTCTCTTTGTAGAACTGGCTGTCCTGGAACTCACTCTGTAGATGAGGCTGGCCTCGAACTCAGAAATCCGTCTGCC
TCTGCCTCCCAAGTGCTGGGATTAAAGGCGTGTGCCACCACACCCGGCTTAATCTTAAATTTTAAGATTATAAAACATAACTGATCTTCAGGCT
CACATCCCCTAAACATTGACTGGCCCCAGCCACCTTGTTTGGACAATGGTAAGCAAACAAATCCATCCAGGGTCAGCGGTGGCCCCTGATACCA
CACATTGTGGTGTGATGGGCAGCTCCAGCAGATGGGATGGGATGGCCTAGTCTAATCCTCTCCTAGTCTCCTTGAAATAAATGTATTTTTCAAC
ACACACATTTTGCAAGTCTTTACCTACCTCAACTTCCACTGAGAATGCAAAACATTTGAGGTCCTTCTGATCTAGAAGGTGAAGCTCTGACCCT
AACTTCAAATTCTCTGGGAGTACATGTCTTAAACTGCTTCTAACCTACCTGACAACAAGCAGGCCCCAAATGGACTGGGGTAGATCTGTGTCTC
AGGCCTGTCCTACTTCTCAACCAACATCCAGGCCGCTGCCCACTTGTTTAACAAGCAGTCATGGAAGACATGTCCACTTTGGATATGTGGGCAG
TATTAATGACCAAAGAGCGTGCAGGTTTCACAAATGCTCAGGCTTACACATCTACATCCACCATTAAATACCACTCATGCTCTGGAAGCTGAGG
TGCCCGTGTGCTCTGTGTGTTGCTGCCCATGGTGGAAGTACACCTGTTGAAGTGCACACAGGCAGATAAGGGGCTGCTGCTATATTTTAGACCC
ACCATGTCTCATCATTTTCCTCCAGCGTATGGGCTGATGCCATTCCATGACCCCAGGATTCCAATTCTTCTGGCCTGTAAAGGGAGCCACCTCA
TTTATTTGCCAAATACTACAGGCTGGAATCCTGATGAAAGGCCACCCGGGTCCAGCTTGTGCCGTCCACCGTTTCCTTTGGCGCCTACTCTCTC
CTGCCGCTCTCACTGTGCTCCTCTATTCTCAGTACAACCTGACACCACCAGCATTCTGTTACACCAGTCCAACCCCTCACCATCCAAGAGCAGC
CAGCAGTCTTTCACCTCTGCCTCTTCACACAGGGTGATCTTGGGACAAGGTGTTTGCTCTGGGAACCCATGAATAGTCTGGCAGGAATTCTATG
AGAACAAAGTGCCTGGTGTGACATTACAGCTGGTCACTATGTAACCCACTGTTCCCTTGCCTGAGACATTTCTAAGCACCCTAGGCAAGACTAC
TCTGCTTGCTGTATCCAAAACGTCAGTACCTGGCAGCTGTCCCACTGATTTGGCAGAAGAAGGGATAAGGTTGCCCCTCACCCACCCCAGTGAA
GTGACCCTGATGATGACCTCAGTATTGGTCTTTACCTATGCCAAGTGCACTTTCGGAGAATGGACAAGATAACTAGTATCCCATGACAATCAAC
TCTCCTCTAAAATACACTCTTCTGGCTAGCTGTCACCTTGTTTAGGTACTGGACAGCCTTCACAAAGCAGTTTATCCCACCCTGGTCACATGAA
ACCAACTTTCCTCTTAGTACTAAGGCAGGACCCAAAGAGCTACAAAGCCTCCCTGATGCCTGCGCCTGAGAGCTTACCTGAAGGGACACCCCCC
CCCCATGTGACAACCTAACTTTGGGCACAGGCCCATCCTGTTTCCTCCTTGTTCTTCCTCCTCCCTTATCATTTAGCCTTTGAAGAGTAACCTC
CAGGTACTGGAGGCCTGAAGCAAAAATGTGTGTAATTGCCTTGATTTCGTCCTGCTACCTCACTCATCCCATGTGACCTTTATTCTGCTGCTCC
TTGAAGCCAGCCCAAATTAAACACTCTAGGAGTCTTTTCAGTACCCTCTTGCCATTCCAAGCCTTGACCTTGACCTTGGCGACTGTCTTTAGCA
CAGTGGTGGGAATGAGCACTTGGTATCTGAGTCTTCAGAGTTTGATGGGGCCCAGTAAAACATTTCACAATGTCAGCTTTGTTCCTTCCTCAGA
AATGGAGAAGCCACCTGCATAATCTAGGCCTTGCCCCCACTGTGTAAAACAGATTTCCCTAGCAACCACTGGACATCTGCTCTTTTAATTTAAG
GCTACACAGGTTACTTGGTATGTGACCTTGGACTGCAGACCATAGACTGGGTGCCTTTGCAGAAGAATATGACTGCTCCCACTCTGGATGTCTC
TCCAGGTCTGCTGGCCATGTGCCTGCCATTTACCACTAGATACCAATCAAGTGTTATATAATACAATAGCAGCATAGTTACAGCCTTCGGTGGG
TGTGGCTGGCGTTCACTCTGGGGAAGAAGTTAGGCTTACAAAACTCATGAGGAAGTTACAAAATTTAAGAAATCCCCGCAAATTCCCACCATCC
TCCCTACCCACTATTTTCCGTCCCCACTGCATTGATGTGTTCTTTTTGGAAGGACAGCACACAGGTCTCAGGAGTTCCCACCAGAGAACATCCT
GACTGCAGCAGGTATGGGGGGCTGTGGGGAAGCGTGGAGATTGGGCTATACAGGAAGGGCAATAGTGTGAACATCTTAAAACTACAGTGCTGGG
AGTCGGGACTCCGAAAACACCTACTGGTGCACTGTGCACACTGGTGGATATCAATGCACGAGCATGTCCACCAGTTCAGCATATCCCTGGCTCA
GCTACAAAGTCGGTGGCCTCCCTCTTTCCCTCCTTACAGCTGTGGGTGAGGCACGCTATTTACACAGTCGCTTCTCAACTATCAAGACTTTGTT
TTTCTTTTTGAGATAGGGTCTCTAACTATGGTACCCCTGGCTGGCCTCGAATTCCTAGAAATCCGGAGCTCTCATCTCTGGGATTAAAGTAGTG
AACCATTGTATCAACCAGCTGTCAGGTTTGTTACTTTCAGGCTCAAGTCCTTGACAGTTGCCAAAGGGTGTCAGGTCACTGCTGGTGGGCCTGA
GAGCCATATGAGCGCTGTCACCCGAGTTCTGCTTCTGAAAGATACCAGGTCCATGCGCTGGACATTAAGAACAACGTGGGAACAGGAGCCTGGC
ACGAAGCCCACCTTCCCACAAAGCTGGCCCGCGTTGTCCACATGGTCCTGGAGACCTCGCCAGCAAAAAGCAAACTGAGTGTGACGTAGGACAG
GAATAAAGATCAGACCGCGAGGCAAGGAGGCAAAAAAAGAAAGAAAGAAAGAAAGAAAGAAAGAAAGAAAGAAAGAAAGAAAGAAAGAAAGAAA
GAAAGAAAGAAAGAAAAAGGAGAGAATCTGGTGGTTTTCTCTGTTGCTGCTGGGACTGAGCTTTTTGCCCCTCCTCGGTTACTCTTTTTTTTTT
TCTTTTTTTCTTTTTTTTCTCCCTCACCCCTCTGCCCCGCCTCCCCTTCCGCCCCGACTCCTCCACCCTTTGGCCCCGCCCACTCTCCCGATCT
TCCCCTTCCCTCCGGCTTAGTCTCCTTAGTTCCGTCCCTTTCCAGCTCTGCCCTCTTGCGACCTGTCCCTCCCACTCTGCCTGCCCCTTCTCCT
ACCCCGTCTGCCCCTCCCCTTTCTCCAACCCTTGGCCCCACCCCCCCGGGCTGTCTTCCGCTCCTTGGCCCCATCCCATTCCGCTATGCCNNNN
NNNNNNNNNNNNNNNNNNNNNNNNNNNNNNNNCACCCCCCAGCCTCTGATCCTTGGCCTACAAAGGGTTTTGAAAAACCCGAAGTTTGCGTGCA
CGCCTGTGTAGGTGCGGCTGCGCCGCTCTCCCCACGTGCCCTGCAGCGCATGCGTGCGGGCTGCGCTTGCGCACTGACAGCCGCGGCGCAGCGG
GGCTTGGTGGTGCCGGTCTCTGGACCCACCGGCCATGGGGTGGCGGGCGGCTGGGTCGCTGAGAGCCAGGTAGCAGTAGGGCAGCAGGGACCGC
CTGCTCGGCCCAGCATGTAGACGTCCACGCGGGCGCGCGGCAGCCGACGGTGACTGCGGGGCCTGGGGTCGCCCTCTGCCCGGCGTCACTGGCC
GGGACCCGTGAAGACACGGTCACGGCGCCGGCCCTCCCCACCTGCCCGAGGCCGGGCGCTGGTGGCTCTGACGCGCCTCCCTGCGTCCGAACGT
GGGTTGCAGCCAGGATGACGCGTGGTCTCGGGGAAGGAGCCTAAGGCACAGGTCTGAAGATCTGGTCCGGCCTTGGGTTTAGGGGGACGAGTCA
GAGGTTCGGGTCCCGGGAGAAGCCATGTAGGCCGCCGTTCTGTTCTAAGCCAGTTCAGCGCGCAGCACCGCAGCCGAATCGGCTCCATTGGTCT
TCCAGTGGGGCCTTAGCAAGCGCCAGAGACCTTGGCATGTGGGCAAAGTAGTCAGACGAGCGTTTTGTAAGCAATCGAGGTGTGAAACTCTCCA
AAACCCCATCGTAAACTTGGTATCCAGTACTTGCCAGGAGCTTCCAAAGTCCTGAAGAGTCCATGGAGATGTTCCTAGAGGCTGCTGCCTCTCA
GGATGTCACACTCACAGGCCGCTTGTCTTGGCCTCTGGCAGGTTTCTTCGTTATGGGAACAGGGCGTGCCCAGCGCAGTCCCAGGATCTGCCTT
TGCTTGGCCAAGATCCGGCGTCTCTTTCCAGCTCTCCCAAGCAATGGGCTTTCTCCTGGCATTGATTCTGAATAGCGACTTGTTTTTCCTTTCT
TGTTTGGGAAACTCTCTGTTTTGGTTCAAAACTTATTCGTTTGGCTTGCCTCTTGAGAAGAGTGTTATTCAAACTTATATTCATTGGTTTCATG
TATCACTGTCAGTTTCCACGGGCGGGAAAGCTGGCATCCTCGCCTTGGTAATTAAAAGCACGGCACCTTCCCTCTGAACGTATTTGTCCTCAGC
AGTGACTTCACTGTGACTTACACTGCCCATCTTGGGCTCTGGGACAGCCACGGGTGCCAATATACAGTATGACCAGGTAGCTTCTTCATTTGGA
GGTGGCATCTCAGAATGCTTCTGCATTTTCTTCTTTGTAGATTTATTTATTAAAAACGATGCAGTTTTATTCATTTTTTTTCTTTTTCATCTTG
TTTGAAGTGCTACAGGTCCAGCCCAGATCCTCACACAAGTGCTGTCCCACTGAGCCACAATCCATCCCCCACCCCTCCTTTTGGGTAGAAGTGG
CTAACAGATAAAACAGCAGTACTTAAATGAATAGTTTAGCTCTCTAGGCATCACTTGGAGATGGAGAGAAGCAAGTGGTGGTTTTTTCCTTTTT
TTTTTAATGGGAGTGGACACTTGCTACTCTACTGCTCATGAATGGAAGGAAGAGCTTCCAAGTGTTGGTTTTCTCCTCCTGCTCCATGGGCTCC
ATGGATCAAACCCAACACATCAGGCTTGGCAGTAAGTGCCTTTGTCTACTGAGCCATCTCTCTGATCCTTCCTTTGTTTTTAGACATAACTTTA
TGTATGCCAAGCTGATTTCTAATTCCCTGTGTAGCCAAGGGTGACTCTGAACTTCTTGTCCTGTATGTTCCTCTGCATGTTGGGATTACAGGCA
TGAACCAAACCATATCCAGTTGGGCTTTTTCTTGTTTTAATGTGATGTTGGGATGAAGCCCAGGGCTTTGTGTGTGCAAGCACTGTACCAACTG
AGCTCTTTTTCAAGCCTTGGGTGGTTTCACTCTTTTTTTTTCTTTCTTTCTTTCTTTTTTTTCCAGGGTGGAGGATGGAGTTCAAGACAGTGTT
TCTCTGTCCTGGAACTCACTCTGTAGACCAGGCTGCCCTTGAACTCAGAGATCCCCTGCCTCTGACTCCTGAGTGCTGGGATTAAAGACGTCCG
GGCACCACCACCCAAAGTCCTACTCTTAATGCTTTTTTTTTTTTTTTTAAAGATTTATTTATTTATTTTATATGTATGATGCACCAGAAGAGGG
CATCAGATCTCATTACAGATGGTTGTGAGCCACCATGTGGCTGCTGGGAATTGAACTCAGGACCTCTGGAAGAGCAGTCAGTGCTGTTAACCAC
TGAGCCATCTCTCCAGCCCCCACTCTAAGATTTACTTTATTTTGGATTTATGTGTATGCATGTGTGTTTGCGTGTGGTTATTATGTGCACACGA
GTATGTGTGCCTTGGAGGCAGGAAGAGGGTGTCAGATCCCTTAGAGTTGGAGTTACAGATGGTTGAAAGCTGCCGGATGTGGGTGCTGGGAAGG
AAGCTCAAGTTCTCTGGGAGCAGTAAGAGCCTTGTAACTGCTGAGGCTTCGCCCTCTCAGGTGTTGGGAATGGATTCATCTCCATCTTTACCTT
GAATCTCTCTGTTTTCTCGTGTCGCAGCAAGAGGGAGATCGGTGCAAAATGCTCAGCACGCGGAGGAGGAGCCTTGGGAGGACAAGAGGGGTGG
GCTTTGATCTCAGGCTCTGCTTTCTAGGACTTGTGAGATCTGTTTACAGTTACCACATGGCTCCTGCCCTCCTGAAGGCAGAGGTGTCGCTTTC
AACCCCAGGAAACCATCCTAATTGTTTGTGATAGTTCCTTAGCTTATCATTGTCCTCTATTCTGATCCATGGCACCTGTCCTCCCAGGGCTACT
GCATTGGGAACAAGTTCGTGAAAACAAATGATCGGGTTAAGGCAGAGACGGTACTAGCAATGTACGCCAGTGCTGTCCACGGTGCAGTGGAAAG
GGCAGCAGCGCCTGACCACTTAGCCTTCTAGACCAGAGTGCCTCTCTTGGGTCTGTGAAACTATTCCACCACTAGATGGGTTCTGTGATCCCGT
TCCTTAGAACTTTCCACAAACAGCACACTTCTCTCACCTGTCTGCTAGCTTGGGTATCATTTATTTGTAGAAATGAGGTAGGTATTAGGTAATC
ACTAAACCTGGACTCTGTGGCTCACAGTAGTGATACCATCATGTTTGATACAGCAACACGTTGTCTCATAAAACAAATGAACAAGAACTGGAAA
CTTTTCCAACTTGGTATTAACTAATGAAGTTAAACATTTACCTGTTGCTTTCACTAATTTTCCTTCTTTTTATTTTTTTTCTCTCCATTTCTTC
CTTCATTCCCTCTATTTTCAAAGACTCAGACTTTTGTCACACAGATTCCAGTCTTCCTCCTGCGTGGCTGAGTCCTTGGCTCACGGTACGCACC
AACACACCTGGCTTCGCTTTTCATTTACCGCTTCTACATATATTTGAAGTTCACATGGAATTGAACGTTTGTATTACGTTTCTTGGGCTGACTC
TCTGTGTCTTCATCCCTGCCTTTGGTTCCTGTTGTACATGAGCTTATTGACATTTTGATATCTTTCTTGCTACTCCATGATGGTGACTCAGTTC
CTTTTCCAAGCCAGCCAAGCTACTCAACAATGAAAAAAATCCCAACAGGGATAACTAAGTGCACTCACCCCCCCCAGCTACCTCAGCACCTGAT
AATCATCTTGCCTAGCCTTTGTTTCCGCTTCACTTTCTAGACCTTGCTTGAAGAGTTACCTAGGGAAAGGTTGGCGACGAAAGAATTAAATAAT
GGAATTAAATAGAATTAAAAACACAGGAGGAGCGATGATGGGTCAGTCCTAGCAACCACCCGGGATTCACTCCCAGGGACCCTCAGGGTAAGAG
GAGGGATTTACGGGCTGCCCTTCGATTGCCACGGGCAGATTGTGTCATGCGAGTGAGCATGTGTGCACCAACCCATAAGTAAATACGTGTATCT
GTTTATTTACTTAATTGGGTTTTTTTTTTCTTTTAAAAAAAAAGTGGGGGAGGGGGTGTTGGGATGTTCAAATCAGGGTCTCTCTCTCTGAAGC
CCTGGCTGTCCTAGAACTCAATCTGTAGACCAGGCTGGCCTCACTTTAGAGAGCCACCTGCTTCTGCTGGGATTAAAGGCTTTTATACCACGAT
CTCCTGGTTTAT
MOUSE SEQUENCE—mRNA
GCTTGGATTCTGAAGTGTGGAAAGCACTGAGACGTGAAGATGAGTGAACTTGACCAGCTGCGGCAGGAGGCCGAGCAACTGAAGAACCAAATTA
GAGATGCTCGTAAAGCGTGTGCCGATGCGACTCTTTCTCAGATCACAAACAATATTGATCCAGTGGGAAGAATCCAAATGCGGACCAGGAGAAC
ACTGAGGGGGCATCTGGCAAAGATTTATGCCATGCACTGGGGCACAGACTCAAGGCTCCTTGTCAGCGCCTCTCAGGATGGAAAACTCATCATC
TGGGACAGTTATACCACAAACAAGGTTCATGCCATCCCTCTGCGCTCCTCTTGGGTCATGACCTGCGCATACGCTCCTTCTGGGAATTATGTGG
CCTGTGGTGGCCTGGATAACATCTGCTCCATTTACAACCTGAAAACTCGTGAAGGGAATGTGCGTGTGAGTCGTGAGCTGGCGGGACACACAGG
TTATCTGTCCTGTTGCCGGTTCCTGGATGACAATCAGATAGTTACCAGTTCTGGAGACACCACATGTGCCCTGTGGGACATCGAGACTGGCCAG
CAGACAACCACATTTACTGGACACACTGGAGATGTCATGAGCCTGTCTCTTGCTCCTGACACCAGACTGTTTGTCTCTGGTGCTTGTGATGCTT
CAGCCAAGCTCTGGGATGTCCCAGAACGGATGTGCCGGCAGACCTTTACAGGACACGAGTCTGACATCAATGCCATATGTTTCTTTCCCAATGG
CAATGCCTTTGCCACTGGCTCAGACGATGCCACATGCAGGCTGTTTGACCTCCGTGCAGACCAGGAGCTCATGACCTACTCCCATGACAACATT
ATCTGTGGTATCACATCTGTTTCCTTCTCCAAGAGTGGCCGCCTCCTCCTTGCTGGGTATGATGACTTCAACTGTAATGTCTGGGATGCACTCA
AAGCTGACAGAGCAGGTGTCTTAGCTGGACACGACAACCGAGTCAGCTGCTTGGGGGTGACTGATGATGGCATGGCTGTGGCAACAGGGTCCTG
GGACAGCTTCCTCAAGATCTGGAACTAAAGCCAGGAGCAGGTGGATGGATGCCATGGCGACTGGAAGACCATTCCAACCTTGAATCGTTACAGC
GATAGCATATCCTATCCAACCATACTAACGTGGACACCCACACCTCCCCTCAGAACTTCAAAAGGGCAAGATCTTTTTTCCTTCACTTATTGCT
GAACCAAGAGCACAATTCCCATTAAGAGAAGGATCTCAGTGCTGTAAACTAAACAAATTGTGCATTTTTCTGGGCCATTGTTTTTGGTTTATTT
TGTTATTTTGTTTTGTTTTTGTTTTTTTGGTTTCATTTTGTTTTGGGTTGGTCCAATTTTAAAAGGAAATACTACAATAAAAATGTTAACCAGA
MOUSE SEQUENCE—CODING
ATGAGTGAACTTGACCAGCTGCGGCAGGAGGCCGAGCAACTGAAGAACCAAATTAGAGATGCTCGTAAAGCGTGTGCCGATGCGACTCTTTCTC
AGATCACAAACAATATTGATCCAGTGGGAAGAATCCAAATGCGGACCAGGAGAACACTGAGGGGGCATCTGGCAAAGATTTATGCCATGCACTG
GGGCACAGACTCAAGGCTCCTTGTCAGCGCCTCTCAGGATGGAAAACTCATCATCTGGGACAGTTATACCACAAACAAGGTTCATGCCATCCCT
CTGCGCTCCTCTTGGGTCATGACCTGCGCATACGCTCCTTCTGGGAATTATGTGGCCTGTGGTGGCCTGGATAACATCTGCTCCATTTACAACC
TGAAAACTCGTGAAGGGAATGTGCGTGTGAGTCGTGAGCTGGCGGGACACACAGGTTATCTGTCCTGTTGCCGGTTCCTGGATGACAATCAGAT
AGTTACCAGTTCTGGAGACACCACATGTGCCCTGTGGGACATCGAGACTGGCCAGCAGACAACCACATTTACTGGACACACTGGAGATGTCATG
AGCCTGTCTCTTGCTCCTGACACCAGACTGTTTGTCTCTGGTGCTTGTGATGCTTCAGCCAAGCTCTGGGATGTCCGAGAAGGGATGTGCCGGC
AGACCTTTACAGGACACGAGTCTGACATCAATGCCATATGTTTCTTTCCCAATGGCAATGCCTTTGCCACTGGCTCAGACGATGCCACATGCAG
GCTGTTTGACCTCCGTGCAGACCAGGAGCTCATGACCTACTCCCATGACAACATTATCTGTGGTATCACATCTGTTTCCTTCTCCAAGAGTGGC
CGCCTCCTCCTTGCTGCGTATGATGACTTCAACTGTAATGTCTGGGATGCACTCAAAGCTGACAGAGCAGGTGTCTTAGCTGGACACGACAACC
GAGTCAGCTGCTTGGGGGTGACTGATGATGGCATGCCTGTGGCAACAGGGTCCTGGGACAGCTTCCTCAAGATCTGGAACTAA
HUMAN SEQUENCE—GENOMIC
AAGAGGGCACTAGATCTCATTATAGATGCTTGTAAGCCACCATGTGGTTGCTGGGAATTGAACTCAGGACCTCCGGAAGAACGGACAGTGCTCT
TAACCTCTGAGCCATCTCTCCAAGCCCTTTGTTTTTTGTTTTTGTTTTTGTTTTTTTAATTTCTGATATTAGAAATTTCTTTTATTTTTTAATT
AGATATTTTCTTCATTTACATTTCAAATGCTATCCTGAAAGTCCTCTATACCCTCCCCGCCCCCACCCTGCTCCCCAACCCACCCACTCCTGCT
TCCTGGCTCTCGCATTCCCCTCCTTTGTTTCTTTTTTGAGTCAGGGTTTCTCTCAGTCTCTGTAGACCAGGCTGGCCTGGAAGACACAAAGAGA
TCTCACTGAGATTTTTATACACACTCAGAGAAAGACACAGACACACAGGGGTGAGGGTGGCTCGTATTAAAGGTGCTGGCCACCACTGTCCAAC
TAGGATGTGTTCTTAAAACATTCTAGGTACTGGTTGTTTATAAGGCTTGAGGCAGAATTGCTTGAAGATGGGAGATAGACCAGCAAGAAGGCCA
TGAGGCCTAAAGGGTGTTTTGGGCGTGGGGTGGGGCAGGGGAGAGGGTTGGTTGTTTTATATCAAGGCCCTTCCATTCCTAATTAGCAAGGCTT
CATTTAGGTCCCAGATAGCTTCTAGACTTGGAAAAAAAGTTTTTTTTGTTTTTGTTTGTTCGTTCGTTTTTTTTTTTTTTAAAGCAACAAGGGC
TCTTTTTTTTTTTTTAAAGATTTATTAATTATATGTAAGTACACTGTAGCTGTCCTCAGAGACTCCAGAAGAGGGCGCCAGATCTCATTACGGA
TGGTTGTGAGCCACCATGTGTTTGCCGGGATTTGAACTCAGGACCTTTGGAAGAGCAGTCAGTGCTCTTACCCACTGAGTCATCTCACCAGCCG
TTCGTTTTGTTTTTTTTACCACTTGGACTACTGACCTAGGTGGGATTTGCCGCTGGGGTTAAGGGAGTAGGATGAGGTCTATAAAGAACTGAGA
GTAATAAGTGCTCCTGAGGCAGCAGAGCCACTTTGTTTCTAACGCTTGGGGTCTGAACATTGAAATCACCGTTTATGTGTGGTTTCACAGATTC
AGCTCTTTAAATGGACATGATACTTAGCTTTGAATCCTCTCCATTCTTGGGCTGGTGAGCAGGAAGCCGGCCTTTTCACTATAGCTCACTCTGC
CCCATAGCAGGTACTTCAGTCAGTGTGGGGGAGGGAAGGAAATTGTTTGTAGAGGAGAATTTTAAAAATAAGGCATTGCTGAATTTTTAAAAAG
AAGCATTACCTTTCTCAGGACTCAGCCCTGACAATTAGCTCCATTAACACACAAATACCTGTCTCCACTGCAAAAGGGTAGAAAAACTTCCCAT
GCTATTTGGTGAAAATTGAGTTGTGCCCAGAAACGATATCTTTGTTTCTAAGCACAAGTTGTACTTACAGTTCCTTCCCTTTGGAGAGGGCATG
CTGCCACTTTCATCTGACTTCAGGTGAGTTTCCAGAGGATCATACCTAGAACAGTGTTTAAGAGTTTCGAAGAAGCCAGGTGTGGTGGCGCACG
CCTTTAATCCCAGCACTCGGGAGGCAGAGGCAGGTGGATTTCAGAGTTTGCGGCCAGCCTGATCTACAAAGTGAGTTCCAGGACAGCCAGGGCT
ATCAGAGAAACCCTGTCTCGGAAAAAAAAAGAGTTACGTTGTTGCTGATAGGTACATATGCCATGTACATAAAACTTTCTTTCACCCACTTTTC
GAGTCATGCTGATACCTGGCCATGGCCTTCATGTAGTGTGAGAAAGTGGAGAGAAAGAGTGGGCAGCAAGCCAGGATGTTAAACTGTTTTGGGG
TGTGGGGTGGTGTAGTAAAATAAACGGGAAGTTTGACAAGAGAATTAAAAGACAATTCCTGAATATTAGTTTTTTTCACTTCTGAAACATGGAT
ATATAAGCCCCAAGATCCGCCATTTAGATGTTTAAACAAATTTGGATAAGTACCGAGGTTGCATCATTATGAAACTAACCAGAAGTGGCTGTGC
GCTCCAGTTGTACCGGAACAAAATGATGCTGGCACAGCAAGCTTCATGTCCTTTAGAACTTTATTATAAAGTTGGAGATGTTTTTTAGATACAT
TGGTATATTTGCATTATAGACTTAATGAATGTATGTAATGCCAAACAATGCTAAAATACTGACTTTTTAAAAAAAATATTAGTAAGTTAGGAGT
GTTGGTGGGTAGTTCAAGGTAATCCTAAACTACCTAGTGAGTTTGAAATCAATTGGGCTACATAAGACCTTGTCTTTAAAATATCAAAATAATA
ATTAAAGAAATGATTTAAACATACAGACTATATTACACTATATTACAAAGATCTCTCAGTTTATATTTACAGAATTCCGAGTTATATCTAATTG
CCTTTCTTCTCTTTCCTAATCCTTCTTTACTCTCTTGAAGTGGTTTTAAGGCAAACCCCCAAATCTTGCCCCCCCCCCCCCCGCCCCAATGAAT
AACAAGTTAGGAGTTCAAATCTGGTAGGATATCTAGCAGGAAAACAATAGTTTTCTTCTGTTTTCTGTCTGATTTTTCCCTTCTAAAAATGTAT
ATACAGCTGAATTTTTTTTTTTTTTTTNNNNNNNNNNNNNNNNNNNNNNNNNNNNNNNNNNNNNNNNNNNNNNNNNNNNNNNNNNNNNNNNNNN
NNNNNTTAATATGTAGCCCAGGTTGACCCAGTTCTCACAGTCCTCCTGCTTAGCTTTGTTTGTTTGTTTGTTTGTTTTTTTAAGATTTATTTAT
TTATTTATTATATGTAAGTGCACTGTAGCTGTCCTCAGACACTCCAGAAGAGGGAGTCAGATCTTGTTACGGATGGTTGTGAGCCACCATGTGG
TTGCTGGGATTTGAACTTCTGACCTTTGGAAGAGCAGTCGGGTGCTCTTACCCACTGAGCCATCTCACCAGCCCCTCCTTAGGTTTTTGAGTGC
TTAGATTAGAAGTGTGTCCCACCTCACCGAATTTGAAAATGTTAATTATTTAGGGAACTATTTACAAATAAATTCAACACTAGCTCATAGCTGA
ACTTCTTATATACGTCCCTATCCATTTCTCTACCTTTTGCTCGTTTTGAGGAGGCTTTTTAAAAATTAATTAATTTATTTTATATATGTGGGTA
CACTGTAGCTGTAGATGGTTATGAGCCTTCATGTCGTTGTTGGGAATTGAATTTTAGGACCCCTGCTCCCTCTGGCCCAAAGGTTTATTTATTA
TTATACATAAGTACACTGTAGTTGACTTCAGTCAGGCGCACCAGAAGAGGGCATCAGATCTCATTACAGGTAGTTGTGAGCCACTATGTGGTTG
CTGGGATTTGAACTCAGGACTTTTGGAAGAGCAGTCAGTGCTCTTACCCAGAGTCATCTCACCAGCCCCCAAGGAGACTTTTCTTAAATACTTT
TTTGTTCCTTTAAATATAATCGTAAACGGTACTATTATCACACTTTAAAATGTTAGCAGTTGCTTCTTATATAGCCCCTCAGCACACTTGTTTG
CTTGGGTTTGGCTTTATGGAGTTGGTCTAATCTTACACCTTCCTTCTTCCTCCCTCCCTCCCTTTCACATTGAGACAGTTTTTCATTTTGCAGT
CCTGTCTAGCCTGAAACTCACTATATAGACCAGGCTGGCCTTAGACTCACAGAGATCCAACTATTTTTGCCTCCCGAGTTCTAGGATATAAAGC
ATTGACCTACTATTCCCAGCCACGATCATAACTTTAAATCTTTTAGAAATTCTTGTCTTTGGAAATAGCATCTGAAAGCTTGGTGGTAGTGACT
GGTGGATACATTTAGTCCCAGAACTTAGGAGGCAGAGGTAGGCAGATATCTGTGAATCCTCTAGGCTAGCATGGTCTACAGAAGGAGAGTTCTG
GGATATCTAGAGCTATACAGTGAGACCCTGACTCAAAAAACCAAAGGAAAAAAGAAAGAAAAAAGAAAATAGCATCTCAAACTAGGACAATAGT
GTTCTTTGTCAATGTGACTTCTCAGATGTCAGGCATTAACCTTGGAGTTGTACTGTTGAAGGGGACACTTCAGTAGTATATCACTTCAGTAGTA
TATCACTTCAGTAGTATATCACTTCAGTAGTATATCACTGAGAGGCTTATGGGATGAGGGCGTATCAGACTGATTATCTCACTATGTGATATAG
ATGATGTTTGTTTCTTTCTTTTTCAAGGCAGAGTTTCAGGTTAAAGACATGTACATCTCTACCCAGCTTCATAAATAAATAATATTTTTTTCAG
ACCAGAGTGAAAAGTGATTTAGTGAGGCCCTATTTGTATGTTGGAGAAATTGAAATTATTTTTCAAGACATGCACCATTAAATTTTAAATTCTA
GTTTATTTTTTGTATGTGTGCACACAGAGAAATCAGAGGACAACCTGAAGGAGTTCATTCTCCTGGGCAATCAAGTCAGGCTCTGGGTTTGACA
GCAGATACCTTCTGGCGAAGCCATCTTGATGGCTCGTATTTCTCCTTTTCATCAGTGGCAGGCTTGCAGCCCAAGTATCGTCAGTAACTGTGTG
AGATAATGGGGTCCTGAGGTCACTGCTGTGCCTGTCCTGGCTCCTTCACAGGGTCTTGACCAGAGGAGCGCTGTGCTGCTGATGCCTTTGTCAC
AAAGTTGGGGGCTTTATATCTCATACCATCACGTGAACTGTTCTAATCGTGCTGACATTTTATTTAAAGATATAGCATGTTACTTCCAGGAGAT
CACACCAACTTGTTTGTCACATCTTTAGTCCCATGTTCTTACATCATTTAAATTTCCTGAACAGCAGCTCTAATTTAAAGCTGACGGCAGATGT
GGGTCATGGGCAGTCGTGAGCCTCAGCAGGTGCTGTCTTAGTGGATTAGGCTGGAAAGTGCTACCTCAGCACAGCCAGAGCTTACCTGGGAGGC
TCTGGAGGAGGAGAGCTGCCCAGGCAGGCAGACCGGATGGTGGGGTTTCCAGAAGGGTCAAAGGACTAACGACACTAAAGAGAAAGGCAGAGGA
GACGAAACGTAAGTAAGACGTGTTTCTAATTGCTAAAGTTGTGTAAACAGTATTATGGCAGACTCTTCAGTTGTGTGCTGCACTTCACTCAGAC
TGTGAGAAGCTAATATTGGCACAGCAGGTAAAGGCACTCGCTGTGATGTTGCATGGCAGGAGAGAACCAGCTATTTCAGGTTATCCTCTGTCCT
TGGAATATGTGTTGTGGTACATACCCATACACAATTAAATAGGTAAAATGATAGAAGAAAATTACTTTAAAGATCGTTTTCTGCCCAGCAGTGG
TAGTAGTACACGAGGCCAGCCTGGTCTATAAAGCAAGTTTCAGGATAACCAGGGCTATACAAAGAAACCCTGTCTTGGAAAAAACCAAAAACAA
ACAAACAAAAAGTTGTTAAAAAAAAAAAATCCTGCTGTGTGGGAGGCAGAGGGTGACTCAGGCGCAGTTTTCTCCTCCCTCCTTTACATGGGTG
GGTTCTAGGGGGTGAACTAGGGTCCCCAGGCTTGCATAGCAAGGGACTTAACCCACAGAACCTGGAAATCATTCTTTTGATTGAATGCTTTCTT
CATATACAAGAAAAATATGTCACAGTCATCCAGGTAATATTGCTTAAAAGTAGAAAAATGTATTTACCAGAAACCTAGTGAGGAATAGAACTGA
TGGGCTATTGAGATAACTTTGGGTAAGGTTGGTGGCTGCCATGCCTGAGGAGTTTTTGAAGCAGCTCAGAAAGATCCCCTGTTTATACAACACT
GGTTCAGCATAGTTGTATTCGGTCTCATGATGTAAGAGCTTTAGTGTTTATGTCCTGCTCAACTGCTGCTTGTTGATTAGACTAAATCAGGTAC
CTCAGGACTTTGGTTGCTACTTTTTCTTACGTTAATGCTGGATTGGATGGAAGGTCTTTATAGCCAGAGTTCCTTTCAGTCAAGTAAGGAGTGC
TGAAAGGTGGTGATCTTGACTCTCTTAAACTAGTAAGAGGGTTTCTTCTTTGACCCTTTGTAAGGTAAAAACAAGTCAAACTCATGCGAGACAA
AAATGGCAGAGTATCTTTCTGTGTTTTTATTCATTTGGGATTTTATTCTGCCATATAGGAATGAATATTCCTAAATCTAGTAAGAGGGTTTCTT
CTTGGACCCTTTGTAAGGTAAAAACAAGTCAAACTCATGCGAGACAAAAATGGCAGAGTATCTTTCTGTGTTTTTATTCATTTGGGATTTTATT
CTGCCATATAGGAATGAATATTCCTAAATTTTAGATCTTAGGTTTTGTGTTCTAAAGAGAATGGCTCAGTCTTCTCCTGCAAAAGTCATGCCAG
GTACCAGGGAGGCCCAAGGTGTCTCCCTTGGTGAATCAGGATCCACTCAAGAACCAGAGATGGTGGATATAACCTGTAATACTAGCCCTGGGGA
AGGGGAGGGAGTAAGGTCAGAGTAGGTGCCAAGGGAAGTTTTAGTATATACTGTACTGCTTAGCAAGACACTGTCTTAAGAAAGGCAGGAACGT
GACTGGAGAGATGGCTCAGCACTTTTCCAGAGGCTCTGAGTTCAATTCCCAGCAACCACACAGTGGCTCACAACCATCTATACTGGTGGGATCT
GATGCTCTCTTCTGGCATGCAGCTATACATACAGACAGACACTCAACATACATACCATAAATAAATAAATAAATAAATAAATAAATAAATAAAT
AAATATTTAAAAAAAAAGACAAGCAGGATAAGGAGCATCAACAGAAAATGAGAGTATCATTGTGGTCAGCAATCTTTTAGAAGCCGTCTTGATG
TATACCCTGCTTTTCAAGTGAGCAGCAGCAGCAGCTGGTCTAGGAACATGGAAGCCCTCTTTCAGTGCACAGCTTTACCTTGTAGGCCTTGGTT
GCCCGTGCCCCCCCCCCCCATGTTAAGTACTTAGTAAGTCGACCCTTCTTTTTGTTCTGCCAGCAGCAAAGACACTTAGTTGGAAGGGATTCTG
TCACCATGAGCATATAGTGTGGAACTGTAGAATAGAGAAGTAAGGAACGTGAACAGTTCTGGGGTCATCCAGCTGATTTATTGTAGATGTGTCT
TACTCTAACAGTGATCTCAGGACACAGCAGGCAGAGCTCGGTGTGAAGCCAGCCCAGTCTACAAAGCAAGTTCCAGAACAGCTAGGGCCACACA
GTATAACCCTCTCTTGGGTGGTGGTGGGGGGCAAGGAAAAGAAATTAATATTGGTTAGTTAAGCATTTGGTATTTTTTAACTTTGACTTTTGAC
AAAAAGTTGATCTTGATACACAATCCTAAGAGATCTGGAGCCCACTATATTGCCCGAGAGGTCTTAGACTCAAAAGGAATCTTTCTGCCTCTGC
ATATCCCAAATTCTGACATTATGGGTATGCTATCATACCTAACTTTATTATTATTATTATAAAATTTGTGTGTGCGTGTGTGTTTTATGTTTCT
GTAGGTATGCTCATGTATGTATGTATATGTTTATGTATGTATWTACATGTTTATGCAGCTATATTCATCTATGTATGTATGGAGGCTGATCATT
ATTTTATTAGTTGACCTCACTTGTCAACACAGGGTCTTACTGAACCAGAGCAGTGGTTCTCAACCCGTGGGTCATGACCCTTTTTGGGAGTTGA
ATGACTTTCACAGGGGTGGCCTAAGACCACTGGAAAACAGACATTTATATTATGGATCGCACAGAGGCAAAGTTACAATCATGAGTAGCAACAT
AAATAATTTTATAGTTGGGAGTCACCACCACATGAGGAACTATATTAAAGGGTAGCAGCATTAGGAAGGTTAAAAACCACTAGAGGTTTTCATT
TCAACTAGATTAATTTGTCATTGAACCCAAGAATCCATGTGTATCTGCCCACACCATTTTGAACCTCTTTAATTATTACACAAAATTTTTGAAG
ATTGTGTGGGGAGTGCTGGAAGATGATTCAGTACTTAAATGCACTTGGTGCCCTTCCACAGGACCCTAGTTTCATTCTCAATGCCTACATGACA
GCTAACAAACCATATGACTCAGATTCCAGGGAATCTGTATGTGTGTGTGTCAAAAACACTCATATTCTGTGTAATTTGCATGGGGAACTAAGAT
TCTTGCATAAATTATATTGGTATTAAGTTGTTTCACCTTCAATATTATGGTGTTATAATCACATTTATAATTGATTTAATAACCAACATCAGGT
CAGAATTGTAGCCTGTGTGCCCCAGGTACAGTGGTAGATGAGGCCTGCTGTTCTAACACTTAAAGAGTCTGAGGCAGGAGATTTGCTATTAGTT
CGAGGCCAGCATAGGCCACAGAAATAAGTTCAGGCAGGCACGCAGGCATTTCTATGTACAGCTTCCTCTGGGAAGTGAAGTCTTTTTGTGCTGT
TGTTTATTTTGGATTGGGCTTTTGAGACAGGATCTCTGCGGTCTTGGCTGGACTGGACTGGTGCATAGGTCTGGCTGGCCTTGAGTTCGGATCC
TCCCTTTCCTGCTTCCCAGCTGCTGGCTCGGGTTAAAGGCAGGCTCTACACTCTGAGGTATTTTATTTAAGGCATGCTCTACACTCTGAGGTAT
TTTATTTTCTGAGGCAGTGTCTCATGGAGCACAGACTGGCCTTAGACTACTGTGTAGCTGAAGATGACATTGAACTTTTTGCTTCCACATCCTG
AGTGGAATTATAGGTTAGCAACACCTCATGTGGGAATTCATACATTGTAGGTAATCAGTCTACTAGCTGAACTATATCTCCAACCCAGGAGGTC
AGGTTTGTTTGTTTGTTTAACAATCTAGTTTTGAAACAGTCATATCCTAGGCTGGCCTCAAGTTATGTAGTCAAAGATGGCCTTAAAAGATGAC
TCTTGGTTATTTTCCAAGTGCTGGGATTATAGATATGCACACCACCACACCTCATTTGTCTCGGGGCTGGACTCAAATCCAGAGCTTCATGCAT
GTGAGGCAAGCACTGTACCAACTCGACTTTTGCATACTCCATTGAAAGTCATTTTTATAACAGGATCATCTCAGTTGTCTCTGACAATGGGGAT
GTTTTATATCACTGTGTATGGCTTTGCAGCTTTCACAGCCTTATTCAACCCCATATCTAGAAGGTATTTCTGAGCTTCCTGGGACTCCTCAGCA
TAGAGTCTGGGGAGAAATAGAAGACTTTTGAATGTCCCTTTGTCTTTCGGCTCATGATAACTGCCATAGCTTCAAAGTACTGGTTTTCCAGATT
ACCTCAGTCCTGTTTCTTAAAGAAATTACTCCTCCTTTCTCTGTGTGGTACCTATTCTGCATGTCTCTGTTTGTTGTTGTTCTTCTTCTTCTTG
ATTTCTTTCTTTTATTATTTTTTAAAGATTTATTTATTTATTATTACATGTAAGTACTGTAGCTACCTTCAGACACCCCAGAAGAGGGCATCGG
ATCTCATTACGGATGGCTGTGAGCCACCATGTGGTTGCTGGGATTTGAACTCAGGACCTCTGGAAGAGCAGTCGGCGTTCTTAACCACTGAGCA
ATCTCTCCAGACCCTGTTCTTGATTTCTGAGAGACTCAGAATCTTTCCATGAGAAGGACCTGTGCTGTTTTAATATTGATTGATTGATTGATTG
ATTGATTGATTGGAGATTGGGTCTCTGTGGTACAGATTGGCATTGAAATTAAGATCTTCCTTCTTGGCCACCTAAGTAGTGGAATGCACCACCT
CTATTGGCATTAATTTTCTTTTTCAGTCCACCACAATATTTTCAGCAGTATATTAGTACAGTTAATAAAAGTGAAGATTGTGTTTTTAATCCAG
TTCTACATAGAGTTTCAAAAGTTCTGATGAGTGCTGCAGAAAAGGTCTCTGCATTGTGTGATTGTAGCTGTGACGTTTTCAGTTAATCATACCG
TATCTTATGCATTTCTTGCCATGTTTCTGTTGCAGAGCTTGGATTCTGAAGTGTGGAAAGCACTGAGACGTGAAGATGAGTGAACTTGACCAGC
TGCGGCAGGAGGCCGACCAACTGAAGAACCAAATTAGAGTATGTAGTCATGCGGGTTTGATATGTTTCATATCAGAAACATATCTATTGTGTGT
GGATAACAGAGTGAGATGAAATTGATCTATTATATGCTAGTTATATATCTGAATAACTTATGTTGTTTCAGAAATTTTGTTTTCTTATTAGCTA
CAAGTTGTTGCTTGAATGTACATGGCCCATGTCTGTAATACTTACCAAAGCTTTTGAACGTCTTCCTCCCGGGGAACTAGGTTTTGGACCCTCT
GTGTGGCCACTGTGGCCGCTGTGTCAGTTCTGAAGCAGCTCTGAAGAGCACGGCTACTAAACTGAGAGCAGAAATAGATACTTTAAATGAAGGG
ATCCGTAAAGTGGCATTTTAGATGTCTCTGTCTTATTTTTCATAGTTTCGCAATGGTTTGTGCTCCAGGAAGACCATTGGAATGTATGTAACAG
CTCTTAGGCCACCATAGAGAGCAGACTTGTCTACCTGGGTACAAAACTAAATGGAAGATAATTCCATGTATTCTGAGTTGTTCTTTCTGTGCTG
GGGACTAGTGTTAGAAATGGAGCTAAGAGCCCACACATCCCAGGCAAATGTGAGTTTTTTGGTTTTGGGGTTTGTATTTTAGTTTTGTTACTTG
AGACAGGTTCTTACTATGTAACTTTGTCCTGAAACTCAGTGTAGACCAGACTGGCCTTGAATGCAACATTATACAGAGATAAGTATGTGGGGTT
TTTTGTTTGTTTGTTTGTTTTTTGTTTTTCTTAGACTTGATCTAAGAGCATGGAAAAAATGACTTAAGACTTAACAAGAAAATCCCAGTTTTTC
GTTTGTGTAAGGATTTGTATATTTGAAATAGCTTCCTCTGATTTTCATGTTCTTGTCTGTAAGGATATGGCTGTCTGTCCTTGAGCGTCTCACT
AAGCCTTTAGAATGGTTCACATGTCTTTAACTTGTCAGTTGTGTTCTCAGGCTGCCAGGAGGACTAGGTGGAGAATTGCCAGCATCATTTTAGC
CAACTTTTACTTGAATTTAAGAAAACATACCAGAACTGCATAAGTTTTAGAGCAGAGATAAAACAAAATATCCTCTGCTGTAAGCACCTCACTG
GACATTGGTGTCTACGCTGTTCCTCCGTGTGTCAGTTTCTGTAAAGTTAATAATTTCATGTGTCAGGCAAGTTTATAGCACTGAACTATACCTA
GCCCTTGCCTAAAATCTTTTTAGGGTTAGGATTTTTGGAAGTGGGGCATCAAGTTGGGTTTTACTATGGTACTAAGATGATGTCAAAATTTGAG
TTCAAATGATCCCATGTGTCAGTCTCCCTTGTAGCTGGGATTGTGTAGCATGCCTGACTTTCCCTAAACTTTTGCATGACTTAAAAACTTAAAG
CCAACTTAAAGCCAGGGGCTGAGCATTTTAATCACTGCATTTAGGAGGAAGAAGCAGAGCGATAAGAGTTCAAAGTCATGATCAGCTACATAGT
GAGCTCAAGGCTCACCACCAGACCATCTCTCAAAAGGTAACAACAGACTGGAGAGGTGTCTCAACAATTAACAGCCCTTACTACACTTTCGAAG
GACTCCATTAGGTTCCCAGTGCCCAGATCAAGAGGCTCACAATCTTATGTAACTCTTAACTCCAAGGAATTTGACACCTTCTTCTGGCCTCTGC
AGATACTTTCATATCCCACACATAAACACACACAAAATAAAAGTTTAAAGTCAGGTACAATAGGGCATGTCTGTAATGCTATCAAGTCTGAATA
CTGAAGGAGTATTGAAGGTTTGAGGCCTGCGCATGGGCCATATGCTTTTGAGATTTTTATTTCTTTGTCCCAAAGTTCTGAAAATTAAGAGTAT
AGAAATGACAGAGGAGTTTTCAGACAAGGTCTCACTATGTAGCTGTGGCAGTCCGAGAACTCACTATATAGACCAGTGCTGGAGACTAAAGGTG
ACTAAAGCCACCACGCCTGGCTTTTGTGGTTTGAACTTCCTGGTTTTTCAAGACAGTTTCCCTGTGTGACCCTGTGACTGTCCTGGAACTCACT
TTGTAGACCAGGCTGCCTTCAAACCCATAGAAATACCCTTCCTCTGTCTCCCAATGATGAAATCACACTGCTTGGTGTGATTTCAGTTTTTAAA
ACGGGAGCAGGGTGGAGATGGCTATGCTGTTTTAAATTAAGCCAACATGGAGATCAACTGCACAGCATGGAGGGAAGATTTAGGGAAAGGATAA
GAAAGCCCAAGTGTTGAAGAAAGCATACTTAGAACAGATAGAAAGGAGAAAAAGAAATACTCAGAAAAACTAGCGGATTTACTAAGCCATATGG
TTGTAGCTGTGTAGCTGCTGAGACTCAAATCATTTAGCAGTAGATAAAGAGATTTTTAAAAAAGATTTGTTTGTTTGTTTGTTTATTATATGTG
TGAACACTGTAGCTGTCTTAAGACTCTCAAGAAGAGGGCATCAGATTTTTGTTACAGATGGTTGTGAGTCACCATGTGGTTGCTGGGATTTGAA
CTCAGGACCTTTGGAAGAGCAGTTGGTGCTCTTAACTGCTGAGCCATCTCTCCAGCCCAGATAAAGAGAGATAGTAAATCAAAGATAAATGTCA
TGCAATATTTTGATAAGCAGGTTTACATACTCTCTAGTCCCTAGTACCCACCCGCCTTTCTGCATCCAAATAGTGCCCCTGTAATTATAGCATT
CAAGAGGTAAAAACAGGAGGGTCAGAGTTCAAGGCCAGCTTCAGTGACATATCAAGTTTGAGGCCAACTGGGCAACATAAAGTGTTATGTTTAA
CATATGATGTTATGTTAATATATATTAGTAATATGAAATCTTAAGTTTCATCTAGTCCCATGTAAAGTATTATTTCAGTCAATAGATTCTTACT
GAGATAGTTTACAGTACTTACATTTCAGTTTTGTTTTGGTTTTAAAGTTCAGCTCATGCCTCCCTATAAGGGCATTCTACAGTTCTCTCTCACT
GTGTCTGACATGCACAGATAGTTGGTGACCCCCCCAAGACCTCTGTTGTGGACAGCACCAATTTGACTGTTCCTTATAGGGCCACTTTGTTTCA
ACCTTCATGATGAGCATCATCTTCACAAATGGCTCCATGGGTTTGGCCTTGCCTCTCAGCACTGCACTAATTTTAGAGTCGATTGGTGAGGAGA
TGTGAAGGCCATTGAGTCTCTAAATGGGAAGGGAAGACTTTTGTTTGTTTTTGAGACATGGTCTCACTATGTAGCCTTGGCTGCCCTGCAACTT
GATATGTAGACTACTGGCCTCAAACTGCTTGCCTCTGCCTCCCCAGTTTACTTTGGAGACTAAAGCTGTGTGCTACCTTACCTGGCAAGGAATA
CTCTTTAAAAAAGTAAAATTCTGGAGTTAGATACTTAAGTGGTTAAGTTTAATCCCAGCACTGCTTATAACTGTAGCTCCAGAGAATCCAAGAC
TGCCTGTCTTTTGGTTTCAATAGGTACCTGCATACACATAAATTTAAAAACAAAAACAAAAAACTTTTTTTTCTGATATAAGGGAGGAAAAATG
TAAAATGTTCTTTACTAATTGCCTACTAAATACATGCTTGCTAAAGAACTTAGTACAAGGGCTGGAAGGATGGCATAACTCAGTAGTAGTCTAG
TGCTTTCCTAGCAGTGTGGTCCCCAAAGTTACAGGGAAAATACGAGGATTATCTTCACTTTGAAGGCCAGCCTAAACTACCAATCAAGAACCTG
TCTTAAAATCAGAAGCAGGAATCCGGGCCTGGTGGTGTACGCCTTTAATCCAAGCACTCGGGAGGCAGAGGCAGGAGGATTTTTGAGTTCGAGG
CCAGCCTGGTCTACAAAGTGAGTTCCAGGATTGGGTGAGACGGCGAGCACTTTTAATCTTAGCAGTCAGGAGGCAGAGTTAAGTGGATCTCTGT
GAATTCTAGGCCGGCCTGGTCTACTTAACAAGTTCCAAGATAGAATTATATAAAGACCCTATCTCAAAACAAACACAAAATCAGAAATAGCAGA
TGAGTTAATCAATGAAGTAAAGGTTCTTGCCACCCAACAGTGTAAGCCTGAAAACCTGAGTTCCATTTCTAGAGTCCACGTTAAGGTGGAAGGA
GAGAGTACTGTCTCTACAAAGTTCCCCCATGACCTCCATACTCTGTAGCAAGCACACTGATACACATCAGTCACACTCTTGGGTTTGTTTGTTT
GTTTGGATTTTTACCAACAAAGCGTTTGTGGGTGTTTTGTTCTCCCCTGTCCCCCCACTAACACCAACACAGGGTTTCTCTGTGTATGCCTGGC
TATCTTGGAACTTGCTCTGTAGACCAGACTGACTTCAAACTCAGAGATCCACCTGCCTCTGCCACTTGAGTACGTACTGGAATAAAAGCTGTGT
GCCACCACCACCTATACAGTCAATATACTTTCAATTATTAAAAATCAACAAAAGGCTTCTAGCTGGTGTGTTTATACACATACACAGCTTCTCA
AACTCGGGGGCCGAGGCAGAGGATCAGGAGTTCAAGGTCATCCTTAGCCGCATAGTAACTTTAAGACCAGTTTGCTTACACAGTGAATCACACA
AGAAATGGCAGTTAACTGTAAGAAGTGTTGTCTGGCCCTTTGATTTGTTTTTTATGTGAACTTCCCCTGGGGGAAAAAAGGAAAAACAAAAAAA
GAAGCTTGTCATTCTTTTGTTGATCCCAAGGAAACAGGCTCTCAAGTAGATGTTTAATTAAAGCACACACGCCTTAAATACAGAGGAACTGAAC
TTACTACTCTGATAGACCATTGCACACATGGACCAGAATGTAGCGACCCAGAGCTGAGGGTGGGCCCAGACTTTCCAATTTCACTGTGAGACAA
CAGTGCTGCATGATACGGGCCAGCCTCTTCTGTACTCAAACCAAACAGGACAGAATGATTGATTAAATGGATCAGTCTACATGAATCCATCAAG
AAATCATTGTAAAGCCGGGGGTGGGGGTGGAGTTTTTTTTTTTTTTAAAGATTTATTTATTATTTATTATATGTAAGTACACTGTAGCTGTCTT
CAGACACACCAGAAGAGGGCATCAGATCTCATTATGGATGGTTGTGAGCCACCATGTGGTTGCTGGGATTTGAACTCTGGACCTTTGGAAGAGC
AGTCAGTGCTCTTAACCACTGAGCCATCTCTCCAGCCCGGGGGTGCAGGTTTTAATCCCAGCATTCTGGAGGCAGAGGCAAACAGATCTCTGAG
TTTGGGGCCAGCCTGGGCTACACAGAGAAACCCTGTCTTGAACAAGCAAGAAAGGGAGGGAGGGAGGGAAAGGAAAGGAAGAAAGGAAGGAAAA
AAAAAGAAAAGAAAGAAAGAAAAATTATTGTAGCTGGGCACGGTGGCACAGTTCTTTAATCCTGTCACTTGGGGAGGCAGAGACAGGTCAATAT
GTATAAGGTTCAGGCCATCCTAATCAACATTAAGTTTTAGGCCAGCCAGGTATAAGGGACCCTCTCAATCATAAATCAATCAGTCATTCTCAGG
CCAGCAGGGTGAGTCATCAACTAAAAGTATGTCAATATTTATTTTACTTATAAGTGAAAACACTTTAATTAATATGGTAAAAATTAAAGAAAAA
TTACTATAGTATATTGTTTTGTAGGTCTTAGCTACATTGGTATGTTTTATAAATAGAAAAGATTGCTGTTTTGTTACCAGTCTTATTGAAATTT
TATTAACAATTTTAGATTCCTTGGTCTATTCATAGAACTTAGATTTAATCATTGTTTGGCTTAAAAATAAAGTCCTTGCATAATGCATTCTCAA
AGAGAAGCTGTTGGCCTGAATAGCTAGTTGGTTTTCACCCTGTTCTTTCATGCAACCCCTTGTGCTCCCATATAACCTGGGAAAGACAGAGCAG
GTGTCTGTTGGCTGTCGGGAGTCAGCCTTGGCTTCCAGGCTGCTCACTGCTGTACCAGCTCCCAGTGTAGTTTCTCCCGGGCCTGCCCAGAGTT
CTATGGGGTCTGCATGTCTTAGTTAGGTTTAGGTGTTGTGAGTTGTGTCTGTTTTGGCCTGAACAACTCAAAAGTGGCAGTGGACTTGGGACTA
TTAGAGAAGAATTAAGGAACAAGACAGTTTGTAGAGGCTTCTTGAAACTTTGGTAAGGAATGTCGTAAAGTTAAAAGTATGGAATAAAACTAAT
TTCCATTTGTTATTTCATAGGATGCTCGTAAAGCGTGTGCCGATGCGACTCTTTCTCAGGTAAGGAATAATTCCACCTTTCAGCTGTCCGTAAT
GCGCAGTCTGCCGCGGGCTTGTATCATGTGGGGTGAGCCCTTTTGTGAGGTGAGGTTGTAGGGCCACTGTCAGGGCTTTTCATTTGGAGGTTGC
TTTGCTGATGGGCAGAGAGGACAGGAACCACTGGTTCCTAAAATGGGAGTGATAGCTCTACCGTTTGACCCAACAATTTAATCAAACTGATTTT
AAAACATGTACCAGGCTGATGAGATGACTGCTACCAGGCTTGACAACCCGAGTTCCATCCCAGAGCTCACAGGGTAGAAGGAGATTCAGAGGTG
GTTCTCTGACCTCTGTGAGCATGTGCCGCTGTCCTGCCCAAGTAAATACAGTTAAACCGTGGGCATCCAGCTCCTGCTGTAGTGTATCTACTGC
TGTGAGAAACACTTCAAACTCTAAGGGAGGAAAGCATTTATATCGGTTTATACTTCTAGGTAATAATATATCATTGGAAAATCAGGGCAGGAAC
CATAGAGGAAAATTGCTTACTGGTTGCTTTCTCAAACAGCCAAAGATAATCTAATCTGTTTAGAAGTTGTCTTGTTCAGTGTAGGCTGGACCCT
CCTACATCTATTACCAATCCAGACAGTGCTCCATACATAGGCCCACAGAGGAATCTGATCTAGGCAGTGTTTAATTGAAGTTCTCTCTTCTCAG
GTAACTGTGTGTCTAGTTAACAATAAAAACTAGCAACACACACACACCTATACTCACACCTACTCTTGGGTAATCAATGGCCTGATCCTCAGAC
CTAATAGACATAGATTTATTTTCCCTGTGTATCCATGGCAGTGGTAAAGTGAGTATGTTAGGTAAAGGTGGAGATAACTAATAATTTAATAAAG
CAGTAATAAAATGGTAGCTATTCTATGAAATGGTAGTTACATGTGATATCAGATGATCTTAAAATATCTTACTGCATGTACTGGTGCGACAGGG
GATGACAGTGCCTATATCCTTGATAAGAAGAAGCGAGGTACTGAGCCATGTAAAAAGCATAGTGGTTGTGGTGTATTGCTGATAAACATGCTGT
ATACTTAAAGCATGGACTCACTGGACAAGGGAGTTAGGACCCAGGCACTACAGAGTGGATAGTTAATAGTGCTACTTAAGGGAGCAATGTGCAG
CCGGGCAGTGGTGGCGCACTCCTTTAATCCCAGCACTTGGGAGGCAGAGGCAGGCGGATTTCTGAGTTCGAGGCCAGCCTGGTCTATAGAGTGA
GTTCCAGGACAGCCAGGGCTATACAGAGAAACCCTGTCTTGTGGGGGAAAAAGGGGGGGGGGGAAGCAATGTGCAATTTAAAGTTTATGAATTG
GCTGCTGGACAGATGGCTCAGCAGTTTAGAGCACTTGCTGCTCTTCTAGAGGACTGGGATTCTGTTTCCAGCACTCTGTAGTGGTTCACAACTG
TCTATAAATCTTCTGCTCTCCTAGGGCTTCTGCATGCTAAAAATATTCTCTCACATAAATATAGGATTTTCCATTTAATATATTTTTGGTTCTT
GGTTGATATTATATTACACAGCCCACAGAACGCAAACCAAGAGTCTATTTGCTAAAATTAATCTTCACTAATTTTATTTTTTAAGATCACAAAC
AATATTGATCCAGTGGGAAGAATCCAAATGCGGACCAGGAGAACACTGAGGGGGCATCTGGCAAAGATTTATGCCATGCACTGGGGCACAGACT
CAAGGTATGAATGGGGCCTTCCTAAAGCAGTACTTGAAATGTTGCCTATCTGCTGGATTTCTAAAAGGTAATGGTTAGAAAGTTGAATTGGTGT
GGGTGTGATAGCACACTTTGAATCTCTGTACTCAGGAGACAGAAGCAGACAAATCTTTGTGAGTTTAAAGGCCAGCCTGATTTACATATTGAGT
TCCAGGCTAGCCAAGGCTACAATGAAATCCATCTCAAAACCAGTAGGATGGGGTGGGGTAGGAAGTTTGTGGTACCAGTTATCTGTATCCAGGT
CTCATGTTCACAGGTCATACTCAGATTTCCGTTTAGCTCGCTGACTGCACTGCTGTCCCTTGCATAGCAGCACCTGCTTTTCTTCACCCTATAC
CTTTTAGCTGTTGGGTCTCTGTTGTCTTGGCTACTCTGTGGGCTGTGTTTTAAGAGTTGGGCAATGGAAGGTAAAAGGTACTGAGTTTACAACA
GCAGACTGTGGTCCCAAAAACTAAATGTAGACTTCAGTGGTCTCTTCACCCTCACTGGGAAGCCTTATGGAAAATGAACGAATGGCTATCCTTA
TCATCCTTCCACATGGCCCTGCCTGCCATCTACTGGTGTATCATTTAAAGAGATACTACCATTTGGGGGTTCCTTTCTTTTATACTTTGATAAC
TACCCTTTATTGGACTGAGTCTCTCCTGACTTTTCCACTCATAACGAAATAAAGTGCGACATGGTTTGTGTTCTCCTTCCAGACCTAAAGGAAT
CTCAGCATCCTTTTCTTCTGAGTCAGTTTTCATTTCTGCTTAGCTTTCTCTTTGTGAAATTATCTGCTGTAGGGTGTAAATAGCTAGAACAAAA
GATTCTATCTTTAGTTCTCTGCAGGGTCCCATACAATTGCTTGTGCAAATGAGCGCTATCTGAGGCTGTTTAATAATGGAAACAGTCCCATCAA
AAAAGAAAAAGGGGGCGGGGCATTGTGTTCTTGCTGCAGCTGCAGCTTCAGTTAGATAGATCATGGATGTATTCTATTGTTGCTGTCAGAATTG
TTAAGTGCAGATTTGTAGATTTATTGATTGATTGTTTTTTCAGTGCCGGACATTGAACCTAGGGTCTCATGACTGCAAGCAATTGGTGTACCAC
TAAGGTGCACCTCTAGTCCAAGAATAGTTACCATATGGTCCTAAAAATGTGAGATAATGACTGTGTTAATTCACTTAGTTTATATTCTGTAATA
TAAACATTAAACAACTTACAGTCCATAGTTACTGTCAATTAAAAATAAAGATCATAAAAAAAAAACCTAAGCAAAGTTAGAAAATGGACAAACA
ATGGTGGCACTTTTTTTTTTTTTTTTTTTTTTTTTTTTTTTAAACTACAGCCCTTGGAATTAAAGCAATATAGCAAGTTCTAGGACAGCTTGGG
CTACACGAAACCCTGTGTTAAAAAGCAAATAAACCAGGGGCTGGTGAGATGGCTCAGTGGGTAAGAGCACCCGACTGCTCTTCCAAAGGTCCGG
AGTTCAAATCCCAGCAATCACATGGTGGCTCACAACCATCCGTAACGAGATCTGACGCCCTCTTCTGGGGTGTCTGAAGACAGCTACAGTGTAC
TTACATATAATAAATAAATAAATCTTTAAAAAAAGAAAAACAAATAAACCTTGCAAAGCATTATTTAGGGATTGCTTTAAATGAGCATACTTTC
TGGCTAGTCAGTGGTTGCATTTTAAAATCTAAAATAGCTGTTTAAGGAGTTGTTGTGGGTATGCATACCCCTCTACCCCCTTACATCATTTTCA
TATTTTTTGCTCTAATAGTAAAGGGTTGTTATTGAGCTTGGTGTGGTTGCAAATGTCTTTAATCACAGTACTTGGGAGACAGAGGCAGGTAGAT
CTCCATGAGATCAAGGGCAACCTGGTCTACATAGTGCGTTTTAGACCAACCACGTAGCACAAACTTGAAGACCTAGGTTTCGATCTCTAGGACA
CATGTAAAACTGATCATGGCCACATACTTAGAACCCCACTGCTTCAGTCTGGTCGAGGTGAAAGAATTATTGGGATCACTGGCCCTAGCGGAAA
AATGATGAGCTTCCGGTTCAGTGTGAGACTGCTTGAGAGGAATAAGGCAGAGAGTCTAGAACAGGGAAACAGTCTGCTTCCTTTGACTTCTGAA
TAGGAACACACACACACACACACACACACACACACTAAAATTCAGCTTTGTGGGGGTGTGTGGAGGAGTGGCGGGGGTAGGCATGTGGAGGTCA
GGGCATTATGACCAAGGAATCAGTTCTACTTCTAAGTGGGTTCCTGGGACCAATTGGTTGGTTGGTTGGTTTTTTTGGGGGGGGGGGGGGTCTT
TCAGGAAATGTTTCCCTGTGCATCCTTGGCTATTCAGGAATTGTGTAGATAGGGCTGGCCTCAAACTCAGAGATCCTCCTGCCTCTACCTCCTG
AATGCTGCTGGAATTAAATGGTTGCAAGAGCCCGGGATTAGATTTAATGGAAGGGCTTGTTTGATAGGCACCTTTACCTGAGCCTTCTCTTTAG
CCCTAAAATTTAACTTGTAATACTATTCCAGTAGTGCTTTTCCAGGTCTGTGGGGTTTTTAATCCCCTCTCTACAGAGCCTGATGAAGTAGTCT
CTTTGGCTGGTCTTCCTCTGAGTAAACTGAAGACTATGTGGGGTTGGTCTATGAGTGCCTCCTTTTGTTCTAGGCAGCTAATTTGGCTAGTTCT
CAGTTTCTATCTCTGTTTGAGGACAGAATATGAAAAGCTTTAGACAATGAACCGTGTTGATTCCTCCAAGTTACATGACCTGAGATTAGAAAGG
CGTGTCTTCTTTCACCTTCTGGAGGATCAGCTAACAGCTTAGCATTGTCTTAATTCCTCACTGAGTTAGCAATTATTGAGCTCTCTGAAGGTGA
CTTCTTAAATCAGAGGATGAGCTACTAAAGATGTCTCTTGATACCCTTGCTCAGAGTCAAAAAGACCAAGCAGAAATAGAGCAGCAGAGGGGCC
TCATACTGTCTGTCCTATAGAAATGGTTGAAAGGACCTTGGGTATAATGACTGAACTGCAATAGATGGGGAGCTGGAAAGACTTTGAGTGACTG
TTAGCCTTGGCTACACAGTACCCTATCTCCAAGGAAAGTAAAAGAATCGTCTGAGGAGTTGGAAATACTAGGTTGTAGTTGAGTTCCCTGTATT
CTCAAGGCTCCAGAGTTTAGTCCCTAGAACCTCATAAAGAAAAAATTTTACCAGAGGACTGAGGTGCAATTAATTGGTGAAGTACAATTTACTG
TGAATTCAATCTCTAGAACTGAAAAAGAAATAAAAGTTGTAGCTGAAAAGAAACTGGATGTCTGTGCCTAGTTCTGTGATAAATTTTGTGACTA
AGGATGCAAAAGGATTTTATTTCTTGTCACATATATATGTTGTTCTTTATAAAGGAAATTTACATATCAGAACATAGCTTGGCAGGGTATGATG
TAGTACAGCTTTAATGCAAGCACTTTGGAGGCAGAGACAAGTAGACCTCTGTGAGTTTGAGCTCATCCCGGTCTACATAATAAGTTCCAGGACA
GTCAAAGCTACATAATAGAGGAACCCTGTCTCAAAAAGGCAAGTGAGTCAGTCATACAAGCATGGTCTGATTAAAAGTGTCAGAGGGCTCAAAA
TGGCGTGGTTGAGTTTTACCTCAAGTCTTTGATGAGCTTCAGAAGTTCAAATATGCCCAAATATACTTGTGGCTGAAATACCTAGAATGTATAT
ATTTTGATTACAATTTGAAGTTAAAACCTGTATGTCACTATCACATGGTAGACCTGGGAGAGCCTCTCACCCTTCCTTAAGAGCACATTGTGTC
TATCCTGCCTCAGTCTACAATGCAGAGAACTGCAGGTTCCAAACTAGGACAGAGTCATGAGCAATTCATAATTCACTATAATTGGTGGCCTCTT
TTGCCCAACCATGTGCAATTCTGATGTCATTTATCATCTCACTACCACACAGTTTTATAGAGACAGGTATTCTAATTTATCTCTGTCTGGGAAG
TTTGAGAGTTCGTTTTTCTTTTAATTTTGAGGGATAGAGTCTGGGCCTTAAACTTGTTACATAGTGACCTTGAACTAACACTCACACACACACT
CTCTCCCCCTCTCCCTCTCCCTCCCCTTCCCCCTCCCTTCCTCCCTCTCTCTGTCTGATTTATGGTTTCTCCACATCCCACATTTATGAGTTAC
AACGTATGCTACCATTGGAGCTCATGGTTGTGTTCTAGTTTGATTTCTACTGGTGTGATTAAACACTGAGCAAGAGCAACTTGAAGAAGATTTT
GTTTGAGTTTATAGTTGTAGTCTATCATGAAAGGAAATCAGCAAAGACCGCAGAGGAATACCGCTTACTAGCTTTCTCTTTAGGGCTTACTTAG
CCTGCCATCCAGGACTACATGCCCAAAGACGACACTGCCCACAGTAAACTGGCCATGCTCACATCAATTATTAATCAAGGACATACCCATAGGT
TTACCTATCTGCCAATAAGATGGAGATATTTTCTCAGCTGAGGCTCCTTCTTCCTTAGAGGACTCTAGCTTCTGTCCTATCCCACCCCACCCCC
AATATAAATAAAAACAGGGCAGGTGTTTAAAATGGATTAAAATGGTTCCTGAGTGGGTAGTGTTTCAAGCTTTTCCCATAATATTCAAGCTTTC
TTGCTGTCAGTATGAACAAAACTGTGTCCTAAATATTTGACATGTCTCATGTCTCTGTGTATCTAATTTAACTTGGAATAAAAAAGAAACCCGT
CAGAAGCAGGTCAGATCAGTACCATCTCTCGGGTGATAAGAGAAGAGCTGGGGAGTCTGGTCATCCAGGTGTCCCCAGGGCTGAGTTGTTATCT
GACACTCATGGTATTCCATTGCTCAAGCATTGAGATCCTCTTTCTTATTATTAAGTGTATTATATTTTAAGCGAATGTAGCTTTAAAGCTCTAG
TTGGTCACCAAGATTTTTTGTTTCTGTTTTTTCTTTTCCTTTATCTTTTCTCCAGGCTCCTTGTCAGCGCCTCTCAGGATGGAAAACTCATCAT
CTGGGACAGTTATACCACAAACAAGGTGGGCAGTGCAAGACCCTCCCTGGAACAGTGACAGTCTGTTCATGGAGCAATGATGACCTCGGAGGGT
GATAGAAGAGCTTGTGTTCCTGACCCATTGAGCTTGGCACACAGTTGTGCTGTGGTTCCTGTCTTGGCATGTAGATAGGAGCTGAGTGCTTCCT
TGTCCTTGCACAAGAGCTACACTCCACTCCTAGTTTTGACTGGAAACTTTCCTAATTTATTACTAGAATACTGAAATGCAGATTCCATTTAGTT
AGTAGTTTGTTTTGTAGTAACAAGAGATAATCTAAGATGCACTATACTTTAATGTTTAGTGGAGGATTGGGAATGTAGCTCAGTGGTAGAATAC
TTGTCTACTGGGCATAATTCCCTGGATTTGATTCCCATAACCAGAAAAGATAAAAAACCAATGTAACCCAACAGATTGGTAAAAGTCTGTGATT
CTAGTAACTCAGGAGGCTAAGGTAGGATGATCGAATAAACCCAGAGGGCAAACCTGCCATGGCTAGATAGGGGAAAGGAGAAGACAGAGATACT
AGGTAGAATGTTTGCCTCATAGGTACAAATCTCTGAATTAAATTTCCACTTCTCTTATATTGAAATAAATTACACTGGGTATGGTGGTACATGC
TCATAATCTATCATTTGAGAATGTAAGCAGGAGCCTCAAGCATTAAAGATTATGCTTACCCACATAATAAAGTGAAACCAGCCTGAACTACATG
AGACCTTGTCTCTAAAAAATAGTATTCACAAAAAACCAAACAAACGGGGCTGGCGGGATGGCTCAGTGGTTAAGAGCACCGACTGCTCTTCTGA
AGGTTCTGAGTTCAAATCCCAGCAACCATATGGTGGCTCACAACCATCCGTAATGAAATCTGATGGCCTCTTCTGGAGTATCTGAGGATAGCTA
CAGTTACTTACATATAAATAAAAAAAAAAAAAATTAAAAAAAAATTTAAAAAAACAAACAAAAAAGAATACATTTAAATTCTGAGTGGTTGCAG
TGGCCAGTCTATAATTCTAGCATTTGGAAGACAGAGACAAGGAATCCACAAAGCAATCTGGTTATCTAGACCTACAGACGAGCTCTAGACAGTC
CTGAGGAACACTGTCTCACAGAAGAAGGTGGAGAGCAGTCAGGGGAGGGCCCGTGTCAGCCTCAGCCTCCGTGTGCACAAGCACACATGCGCAT
AAAGTACGGGGAGCCGAGCCAGGCGTGGTGATACACGACTTTAATCTCAGAAGTTGGGAGGCAGAGGCAGGTAGATCTCTGTGAGTTAGGGGGC
CACCCTGTCTATAAAGTGAGTTCCAAGACAGCCAGAGCTCTAGAAACCCTGCCTGCAAAAACGAACGAAAAGGTGCAGGGCTGGAAAGATCGCT
CAGTCACTGCCTCATTTTTCCAGAACGTCTTGAGTTTTATCCCCAGCGTCTGCGTACTGGTTTCCAGCCTTCTCTGTGTCAGTTAAAGGGGAAC
CCAGAGCCCTCCCTCCTTTAGCTTTCCAGGCTCTGTACTCAGTTAATCAACAGTTAATCAAATGTTCCTACAGATAACATGAAATTAAATCTCA
AGACAAGTTTTAATGCTCTCATCAATTAAGGAAAAAAAATTGGACAGTTGTATTTTCCCTCAAAATTAGTAAATACATGCCAAGATTACAGAAT
GTTAACTGCTCTATCTAAACAGCAAGACTAACTGTGTTACCAATGTGCAGTTAGGTTATTGCATGTGTGTGTCAGAGAGAATGAATATGAATGA
GTTCTGCCTCAGCTTCTTTATTGCTGGGATCGGCATGAATCACCATGCCTGGCTGTTTTTTATGTGCTTGTCTTTGTTTTTTTTTAAGCTCATA
GCCATGATAAATTAAAGGGGAAACTAAATCTTTTCAAATCCTATTAACATCACTCCTTAAATCATTAAAATGCAAATATAATTTGTGTTTTCTT
TGAGATAGTTTTCCATTATGTATAGCTCAAAATATCCTCAAACTTGCCATCGGGGCTGGTGATATACACCCTACTGGACAAGACTTTTGAGTTC
AAGTCTCAGAACCAAAAAAGACAGACACGACAAAAAGCCCTCTTTTTCTTTCTCTTAGCTTTCCAACTCCTAGATTCACTGACGTATGCCATCA
CACCTATCTACTCATTAGGGTTTTTTGGTTTGGTTTGGTTTGGTTTGGTTTGGTTTGGTTTTGTTTTGTTTTGTTTGTTTTTGAGACAGTTTTA
CTATGGAGCCCTGGCTGGCCTCAAATTCAGAGATTCACCTACCTTTGCCTCCTGAGTGCTGGGGCTACAACATAAACTATACACCCAGCTGTAG
TCATATTTTCTTGAACTTAATAGTGTTTCCTGTCTGTATTCTTGTTATTTAGCTTCATCCCATCCCTCTGCGCTCCTCTTGGGTCATGACCTGC
GCATACGCTCCTTCTGGGAATTATGTGGCCTGTGGTGGCCTGGATAACATCTGCTCCATTTACAACCTGAAAACTCGTGAAGGGAATGTGCGTG
TGAGTCGTGAGCTGGCGCGACACACAGGTACACATCTCATGTTCCAGTGGTGTAAGCTGCTAACTGGGATGTATACTGCTCTTGTGATCTGTTG
ATTATTGGTTCAAGTAAAAGTGAAAGTAACGGCCAGAAATTTTCTGAAAGTGAAGCCAAACCAGGGCCAATATGCCTTCATCTTTAAGAAACAT
GGAAACTGGTTTTTTACCCAGTTTTCGAATTCCCAGCTGCTTCAGCCATTCTTGTTTATCAACGAATTGATGTTTCAGTCCTGTTTTTAAACTG
CCTTCCATGCTTGGACATTAGCACAGGAATTCTCCTAGTCTAAGCAAGTGTCCATATGCACAGCCATATCTGTTTGTGCTCACTGACCAGTGCT
TTTTTAGAGGAGGCCGAAGAGAGTCTGTGGTCCATTCACCTTTTGAATGTAACCAGCATTAAAATGTTGGGTGCATATTTTTGGAATTCATACA
ACACCATTTAGGACACATTGCCTTTCCAAGGAAGAAAGTCTCATAGTATTTTCTATCTATCCTGTCCCGCAGTGATAGGTAAAACACTCTTCGC
ATGTAAGCAAAGGATTGTTCATAATTAACTGTCATGAAGAACATGGCAGTTTTGTGTTTTAACCCTTAGGGTCAGGTAGGATTGCATCCTTTGG
TGCTTCTTAAGTTACATTGCCAGTGTTTATAACTACTTCAGGAATGCTTGCTTCTTAATATTCTGGCTTTAGCTGGAAGCATAAGCCACCAGTT
TTAGATCTCTTCTTTCTCAAATATATCAACTTAAAAGTGGCCAAATCCAGGCTGGGAGTGGTGGCGCACGCCTTTAATCCCAGCACTCGGGAGG
AGGTAGAGGCAGGACGATTTCTGACATCGAGGCCAGCCTGGTCTACAAAGTGAGTTCCAGGATAGCCGAGACTATACAGAGAAGCCCTGTCTTG
AAGGGGAAAGAAAAAAAAAAGGCAAAATCCATACAGGCATGGCTTCCTGTAAATTTTGATCAGTAGTATTTAAATTACTCTTCTAGGGGCTGGC
GATGTAGATTCGTTGGTAGAGTATTTTCCTAGCACACACAGTTCTAGGTCTATCCCAGCACCATAAAACTGGACATCATGATAATTTGTAATCC
CACCTCTCATGAAGTGGAGGTAAGAGGATGGAGAATTCAGTGAGTTTAAGGGCAGTCTGGGCTATATGAAACCCTGTCTTAAAAAAGAAGCATG
GTATAGTTCTCAAGTATATGATAGAAAAAGAAAACAATCGGGGAGAAGTCTCGGTGACTAAGAACAGCCACTGCATAAGCACAAGGACCTGGGC
TCCATCCCAGCGCTCACAAGCAGGCCTGGCGTGTGATAATTTCATCATTGTAGGCTGACTCCAGCCTCGTTCAGTGAGAGGCCCAGTCTCAACG
GACTATAGCAAACAGTGATAGAGCAGGATATTCAATGTCTGGTGTCCTCTGTTAGTCTCTGGATAGGCATACACCACATACACAAATACAAGTA
AGACGGATATCTAAAAATCCTTGGGGGGGGGGGCGTCCTGTTGAATTTGTATTAATTTTTTTAAAGATTTATTTATTTTATGTATGTGAGACAC
TATGGCTGTCTTCAGACATAGCAGAAGAGGGCATCAGATCTCGTTACAGATGGTTGTGAGCCACCATGTGGTTGCTGGGAATTGAACTCAGGAC
CTCTGGAAGAGCAGTCATTGCTCTTAACCGCTGAGCCATCTCTCCATTCCCCCTAAAATATATTATTTAGAGATTTGGAGAGATGGTTAAAAAC
ACTGGCTGCTCTTTCAGATGGCCTGGATTCAGTTCCCAGCACCTGCATGTGCCTTACAGACTTCTATATGTAATTCTAGTCCCAGGGAATTTCA
TGCCTTCTTCTGGCTTCTGTGAACACCAGACATGCTTGTGGAATACAGACACACAGGCAGTCCTGGTTTTATTGCTGTGATGTGACATCATGAC
CACAGCAACTCTTAGAAAGGAAAACATTTCATTGAGGCTAGTTATATAGCTCAGAGGTTTAGTTCATTATTGTCATGGGAGGAAGCATGGCAAC
ATGCAGACAGACATGGTGCTGGAGAGGTAGCTGAGTTCTATATTTGGATTCCCAGACAGCAGGAGAGAGTGACACTGGCCTGTCTTGAGCATCT
GAAACATCCACCCACCGTAGCCCTCTTCCTCCAACAAAGCCGTTTCAACAAGGAGGTGTTCCTAATAGCACCATTCCCTATGGGCCCGTGGGGG
CCATTTTCATTCAACACACCACACAGGCAGAAAATTCATATACATTAAAAATTTTGGGGCTGGAGAGATGGCTTAGTGGTTAGGAGCACTGACT
GCTCTTCCAAAGGTCCTGAGTTCAAATCCCAGCAACCACATGGTGGCTCACAACCATAGTAACAAGATCTGACATCATCTTCTGGAGTGTCTGA
AGACAGCTACAGTGTACTTACATATAATAAATAAATAAATCTTTAAAAAAATTTTTTTTTGAGAAATATTTAACTTACTAGTTTTTAGCTAATT
TTAAATTTGAACTGGTGGTCTACCAAGACATCATGCTATAAGAAACAGAAACTGTATTATCTGGTTGTTGGGCATAGTGTGTGTCTGTCTTTCC
ATCAGAAGGAAAGAATCTACATCTGTGTATTTCAGGTGTTGCATGTACATTTATTGCTGCTGTCTGTATCTCCTTGAATGATCGGTTGTTTTAT
CATGGCTTAGTATCCCCTCTCATAATATACTTTACTGTGAATCTACTTCATGTGGGGTTTTCAGCCAGCTCAGCCTCAATGCTCACTGTCTGCA
TGACATTTTTTCTTGTTGATTGTTTTTCTAATAAGTTCTCTCATACACAGCATAGAGTTAAGTCTTCACTTCTGTTTAAAGTCTCTGCCTTTAG
GAGCTGGGAACGTAGCTCCTTTAGGAGCTGGGAAAATAGAGTGCTTACCTACCATGAATGAAGCTCTAGGTTTGATCTTCAGCCCAGCATAAAA
CCAGATCTGGTGATGTAGCCCTGTAGTTCAAGCACTTAGATAGATGGAAGCTGGGAGTTGAAGGTAATCCTTTACTGTGTAGTGAGTTTGAAAC
TGCCTGAAGTACACGACAGGTAGCTTTTAAAATAAATGGTTGGGGCAAGTCCTGTACCTTGTAAATACTTTACATATAGTTTCTTCTTGACTTA
TAATGAGGTTATATCCAGAAATATCAATCAATCAATCAATCAGTCTCTCTGTCTCTCTGTCTCTCTGTCTGTCTGTCTATCTGTCTATCTATCT
ATCTATCTATCTATCTATCTATCTATCTATCTATCTATCTATCTATCTATCTATCTAGGGTTTCTCTATATAGCCCTGGCTATCCTAGAACTCA
CTCTGTAAACCAGGCTGGCCTTGAACTCAGAAATCTGCCTCCCAAGTGCTGGGTATCCAGAAATCTCTTTAATGTTTAAAATATTAGCCACTGA
ATTATGTTGACTCTGTCTAGCCTGCCATCCCACATCTTCCTCATCTTATATTGACCAGGCTGGCCTGAAATCTGCCTGCCTCTGCTTCCTGGGT
GCTCAGATTGAAGTGTGTGCTGCTTCGATGGGTGTATAAACTCTTTGTAGCCTAGGACATTTGAGGCTGCTGTGTTACATTCTTACAGTCCTGA
CAGACAGCTTCAGTTCATTGCTGATAACCAGTACCCTGGGCAGATTATACATACCACTTATTGATAGGTTGAAAGAATTGCATTTAAAATTTGA
AGTTCATTTGCACTAAAAAAATCCTAAGTGTTGCTTTGGATGTGGTTGGAGTTGGTACTTAGGGTGGTTCTCTCTCCCTCTGCTTTCTTTTCTG
CTGTCTGTCTTTCTTTCTTTCTTTCTTTCTTTCTTTCTTTCTTTCTTTCTTTCTTTCTTTCTTTCTTTCTCTCTTTCTTTCTTTCTTTCCTCCT
TTCCCTCCCTCCCTCCCTCCCTCCCTCTCCCTCTCCCTCCCTCCTTCTCTCTCTTTCTCTCTCTCTCTCTCTCTCTCTCTCTTTCTTTCTTTCT
TTCTTTCTTTCTTCTTTTGGGTTACTCTGTTTCTAGCTCTAGCTTGCTCCAGCCTCTGCATTCTTTTCCCTCTGCCTCCCCAGTGCTGTAATTA
CAAGTGTGCACCACCATGCCTGGATTCAACATTGTGTTTGAAGAGTTGTATAGGCTCACATGCACTCAGACAAACAACGTCCTCATTTCCAGTG
CTGTGTACTCCTTCCTGGGGGGGCATCTCTTTTTCTGGTTTCCTGTATGCCAAAAAGCTACTCCCTCATAACACCTACAGTTGTACATTTTGCC
AGTCCCTTTATGTGCTATTTTTTTTAATGTCTGTATGTTTGTTCAATGAAATTTAATTTACAAATTAAAGAAAAGAGATTAACAGTAAGCAGTA
AGAAGGTAGTGCAATATAGCAATACACTATAATTAAGTTTTTGTTTTTTTTTTTTTTGACTCATGGCACTGAGAATTTTATTTATTTATTTATT
TATTTATTTATTATTTTTTAAAGATTTATTTATTTATTATATGTAAGTACACTGTAGCTGTCTTCAGACACTCCAGAAGAGGGCATCAGATTTC
GTTACGGATGGTTGTCAGCCACCATGTGGTTGCTGCGATTTCAGCTCATGACCTTCGGAAGAGCAGTTGGTGCTCTTAACCACCGAGCCATCTC
ACCAGACCTATTTTTTATTTTTTAAGGAATTATTTGTTTCTGGAATTTGCTTGGTTTCCATGGGTTAACTGAAACCAGATGAGGCTGCAGTGGT
TAGAGTAAACTGCTTGTGAGAAGTCTTGATTTTTTTTTTTTTTTTTTTTTTTGAAAAGATCTGTTTTATAGTAGTAGTAGTAGTTGTTGTTGTT
GTTGACAGGGTTTCTCTGTGTAGCCCTGACTATCCTGGAACTGGCTCTATAGATCAAGTTGACCTCAAACTCACAGAGATCCATCTGTCTTTGT
CACCACCTTTGAGTTAAATTATTTTTATTGCTTTTATTTATGTGGTGGGGGTCAAACATGAGGAGATCGAGGATGATTCTATAGAGTTAGTTCT
CTCCTCTCATCTCTGCATGGGCTCTGAAGATCAAACTCAGTAGGAAGTTCCTTTACCCACTGATTCATCATGCCAGTCTTTGACTTTCATGTGT
GTGTGTGTGTGTGTGTGTGTGTGTGTAATTAAGAGGACAACTTGTGGGAAGTTGGCTTTTCTCTTCCACCATGTAGGTCCTGCAAATTGAACTC
AAGTAATCAAATGGCAGCAAGCACCTGTACACATGGTCCCATCTTGCTGGCCCTTGACCTTTATTCCTGAAGGATAGTTTTGCTGGACATACAG
GATTGATGTCACCTTACATACAGTTGGTGTCACCTTCATGTCTATGTGTGTTTCCCCTGCATAAATGTTGATGCACCACATGCATGGCTGATGC
CAGCGAAGCCAGAGTGAGGGTACTGGGTTTCTCCTGGAACTGGAGTTAATAGTTACAACCCATCGTGTTGGTGCAATGAATTGAATCTGAGTCT
TCTAGAAAAGTAGCCACTGTTCTTAACCACTGAGCCATCTTCTCAGCTTTCAACATTTAAAAATATAACATATTAAAGGTGTATCTTTGATTTT
TGCAGTTTGATGGTGATATTCCTAGACGTGTAACTTTCTTTATATTCTTGTGTTTGGCATTTGGAACTCACTAAAATATGTCTTTTGCTAAATC
GTAAGGTTTGAGCTGTGTCTTCCAATGCCCTGGTTTCTATGGTTTTGGGGTGAGCTGCTTCAGTTTCCACTTCCTCACGTGTTTTGTTTCTATT
CAAGTCAATTCAGTTGACTTTGGTCACTAGACAACAGCTTTCCTTCTTGTTAGACCAGCAGTGAGCAGCATTTGAAGGAAGCTCAGCTGGAGTA
GACGGCAAGGGCACAGACTGGGCTGGGGAGGCATGGAGAGTGCTCACATAAGTGTCCACATTTGTGTCACATGGTCTCTTCAGATTCAGAATTT
CTGTGTTTTTATTTTAAAAGATCCCCTCTACAAGCACTGAGGCTTTATGTCTTCTTATTTATAAACAAGATTATTTCTCATGTTTCTGAATATA
ATTACAAGAGCTAAATGTAAAATGTCAGCTAATTGTAGCATCTGATCTTGAGGTATATCTTTGTGGTCCTTTCCTCTTGAGTGTGGATCACACT
TTGCCATTTCTTAGTGCACTTGCAGCCTTGGCAATATAATTGTCATAGCAACCGTGGGCTCGTTATGTTCCTTTCAGATGTCTGTTCAGTTTCA
TCAGGTTAAGAAACACATACATAGCAGCAAAACTCTTACTTGTCCTCCTGTCACTGGAGCTGTCTGTGGGTTTGCTTGCTGCCTGTGTGCTGTT
GCTGTTAACTATTAAAGTGCCTGTCTGTGTAAGTTAGGACTGTTGGAGGCTGCTTTAGCACTTCTCAGGGATGTTCCAGCAGACTGGCTGATCA
TCCAACTGTGTAAGACAAAACCATTGAAGTCCGTTGAAGAACAGGCGGTTGTCCATTTACGAGTTTCCTATGCCCTCCAGGGTCAAAACTTAAT
AGAATGTATAGGCTATCTTATAGGATAAGGAAACCCTAGAAATGGATGTAATTGGGTTCACAATAACTGAAAGAGTGTTCATTATTACTATTTA
TATTATTATTTATAATTTATAGGTAGAATGTATATTCTTTTTTTTTTTAATTCAAAATAATTAGCCAGGTGCTGTGGCATACTCCTTTAGTTCA
GCACTCAGAAGGCAGAAGCACAGGACTCTGACTTCAAGGCCAGCCTCATGTATAGACTTGAGTTCCAGAACAGCCAGGGCTACAAAGAGAAACC
CTGTCATAAACAAACAAACAAATAAGACAGGCACATACATATGTACATGTGTGTGTGCGCATGCGTGTACGTGCACTTTTTTTCTTTTTTTTTT
TTTTGAAAATGTGACTGTTGCCTAGATACTATTTTTAAGGCGACTGGAACGTAGGCACAGATTGGTCTCAATGCTAATCCCTAGGGACTGATGA
AGAAAGAGTGGAAAGGTGGCCTCAGAGCTGCTGTTTTGAGACCTTTAGTTCTGGTCCCGAGGGCCATGATTTGCCTGCCCTTCTGATGTAACAT
GGCGTTGCCATTTAGTATTGTGACCTCGTAGAGTGAGTAGCTAGAACATTCTGGTGTATAATTTTTTTCATCTAATGAATAATTGGGTGTTTTA
CATATTTATGAATACAGTGGGTTGTCTTTGGGTTTTTTTTTTGTTTTTTGGGGGGCTGTTTGTTGGTTGGTCTGGGTTTTTTTCTCCCCTCCCT
CTGAAGAGTATTGGTGACCTTGGATATAGAAAAAAAGATTCTGTCTGTCTGCAAAGTCAGCTGTGAAACACAGAGCTGGTCAGTTACTTACTTC
TCTTACTGTCCTCTTGGTGTCCCCTCCCTCCATGTCACTCCAGCAAAAAGGAAGTGCTACTTTCAGGGCTAACCACACAGGCAATTGCAGCACG
TACCTGTCCACAGAACCCATACATATCAGGGAAGTTCTCAGTGTCCTGCCTTGTTGAGTGAAATTTTTTTTTAAAGATTTATTTATTTATTATA
TGTAAAGTACACTGTAGCTGTCTTGAGCTAAGACAGGATCACTGCTACAGGATCACTGTAGCTGTCTTCAGACGAACCAGAAGAGGGCGGGTGG
TCGTGAGCCACCATGTGGTTGCTGGGATTTGAACTTCGGACCTTCAGAAGAGCAGTCTGGTGCTCTTACCCACTGAGCCATCTCACCAGCCCCC
ATTGTGTGAAATTTTATGTAACCATTTATAATTTCTCCCTTGGCTTGGCAGATTTTACTCCACTCTGGCAGAACTATGTGACTTTCAGATATCA
GTCTTTTCAGTTACTCATTTTCTTCTCCCTCAAAATTTTTATTGTATTTTGAGGCAGTCTCGCTGACCCCTGTTCCATCCACAGCTTCTAAGAA
ATCTTTCCAGTGGGTGTCAGCAGAAACATAGCTATTCTGTGCATACTCCATATCACAGCTCTTGAGAAAGCAAGCGCAGTCACCCGTATTTTTG
GAGCCTTCCCACTCGATACTTTCAGTGTGCTTGCTGTTCCTCTAGTTGGCCTGTAGCCCTTTCTCCCTTGGTTCCGTCACTATCTCCCTTCTCA
TTGGTTCTGAGCTCCTTTGTGTCCAGCTGCTCTACTCAATAAGTAGATGAGCACACAAATGAGGATAAAAGGAAATGGAAAGACACAGGTGGAA
GGATGAGGTGTTCCCAAGGCATAGCTCACTGCTATGTGCTGTGCAGGTGTGGGTCCGTGTGAATCATGAACCCATTGAGTCTGCCTATAGTTTC
ACCCATCCCGTGACAGACGTTCTGGGCATTTTTATTAACGTGGTTTTCACAGGGAGAGGCTCCTAAGATTGAGACTTTCTAGTCTTGGTTATTT
ATTTGTGTCTGCTCTTTTTCAGGTTATCTGTCCTGTTGCCGGTTCCTGGATGACAATCAGATAGTTACCAGTTCTGGAGACACCACATGGTGAG
TACCTGACAAGGCAGCAATTGAGGCTTGAGGTCTGTCTACTGTTCTTGTTAATACTGGTTAGTAGGGTTACTTCCCATTGCAAGATAAATGCTT
CTTGATCATAACCTGACAATTAGAAAACAAAGTATCAATGAGTAACTCATTGCATCATCTGTGGATATGCAAAGTTTGATTTTGAACAAAGAAC
TAAAATGTAGATATTTAAACTATAGTTGTATATCAAGTACCTGGTTTTTTCTCCATTTATTTTTACTCATGTATGTGGTGTTTTTAGCCCTGTG
GGGTTCTTTTTCACTGTAAAAGTCAACTTAGCATATTGTGAGAATGAGTAAAGCTTTCAAACAGCAGCTCTGTCAGATTTGTTTATTTTAGAAT
GTTTGCTTACTAGTACATGGAGACACATTCAAGTAATTTTTCCCTTTGTTTATTAATGAAATGACCAGCTGGTTATTTTAAGAGAATCATTTTA
AACTTAAGCTGCCCTTGTGGGTTTTTCTGGTCGTGGGAGACATTCCTGGGAGGTCAGTAGCAATATTTTAAATTTGATTTAGTAGCATGGCTTA
CAGGCTGGTGAAATCCAACAATGGACTATTGATGAAAATGTTAGCCAGGCGTGGTGGCGCACGCCTTTAATCCCAGCACTCGGGAGGCAGAGGC
AGGCAGATTTCTGAGTTTGAGGCCAGCCTGGTCTACAGAGTGAGTTCCAGGACAGCCAGGGCTACACAGAGAAACCCTGTCTCTAAAAACAAAC
AAAAAAACAAAAACAAAAACAAAAATGTAGTCAGGTGGACAGTGAAGATTAGTCATTACAAATTTCAAATTGCATGTGTTGATTATTTTCTTGG
TTGAAATATGTGAAACTTTTGTGCTCTGTGCTTCTTCCTTCCAGTGTCCCTCATGCCTAGCAAAGATAACCAGCATTCATTGTAGTGAACTTGG
TTGATTGCATTGGTCTTGCTGGGTGAGACAAGCTGGTGAGGTTGACAAGAATGGACAGTGTATCATGCTGTTGCTTCTGCGGGTTGCAGCAGGA
AGTAGCCTGACTGCCATTTCCATAGCCAGCTCCCGCTTTATGTAATGCTGATGATATCGTGGCTTTTAGCCACTCTGTCAAGCAGAGGCCCTTG
GAAAACCTTGTACATCCATGAAGACCATCTTTGGTTTATGGTCCTCGTTGCAAGAGGAGTTGGAGCATCAGTTTCTCTGAGCCACTTATTTACA
TTCCTAGTGTCTAGGAAGGTGGAAAATGAGGGGATGGAAAGAATAACACAGGTGAAACAGGCTCCTTTCTTTCAGACCTTAGCATTTTCTGCCA
TCCCACAAACTGCCCATGATTATCTTGTGTGAAGGACAGTGGCTCCTTTGCTGTAGTGTGAGGTAACCTGGAAACAGCAGGCCCATATGCTCAT
CCATATGGTTGTTTATTGTTTTCATTTTCTTTTCCTTAGCAAAATCTCATCACAGAAGTAAATGTGTCTTCCCCGTGGCACAGAAGTCATCTGT
GATTCTGGGTAGTATAGAGGATGTTGTTCTAGGTGATTGGTCCCATGTGCGTGCCTGATACCTACTTTTCATTTCTTCCTGCACCAGTGCCCTC
TGGGACATCGAGACTGGCCAGCAGACAACCACATTTACTGGACACACTCGAGATGTCATGAGCCTGTCTCTTGCTCCTGACACCAGACTGTTTG
TCTCTGGTGCTTGTGATGCTTCAGCCAAGCTCTGGGATGTCCGAGAAGGGATGTGCCGGCAGACCTTTACAGGACACGAGTCTGACATCAATGC
CATATGTGTGAGTTTAAATTGTCTGCTTTAGATCTCAACCTTTGTTGTTGTTGTTGAAGATTTGGGTTTTTTTTTAGGGTCTGGAGAGATAGCA
TATTAGTTACTTTTCTGTTGCCGGAACAAAAACACCATGATCAAGGCAACCTAAAGAGAAAAAACATTTCATTGAGGCTTACAGTTTCAGAGGG
CGAATCCATGTCCATCACTGTGAGGAGCCTGGCTGCAAGCAGGCAAGCATGGTGCTAAAGAAGTAACTGAGAGCTTCATCTTGAGATATAAATA
ATCACAAGGCAGAGAGTGAGTGAGCGAAAACTTGGAATGGCATGGCCTTTTGAAACTTCAGGCATCCAACAAGGCCATACTTTTTAATACCTCC
CAAACAGTTCCACGAACTAGCAAATGAAGTGTTCAAACTTGAGAGCATGTGGGATCCATTCTCATTTATATTTGTGTCTGCCTGTCTGTGTATC
TCTATCTGGGTGTGTGCATGCGTATTAGAGCCAAGTAGGCTGTTAGGAGACACCTGATATAGATTGGGTGAACCAAACTTGGTTGTAGTAAATG
GCTTTTAAAGTACCTTTAAAGTACCACTACCTTTAAAAACATTTTTTTTCAAAGTTTGTTTTAAAATTTGTGCTCTGGTTTTTTGTTTGTTTGT
TTTGTTTTTTCTTTGTGTTTATTCTGAGAAAAGGTCTAGTAGTATAACCTAGGCTTACCTAGAACTGGAAGTCTCTCTAGCACTAAGATCACAG
GCAGGCCTTTCTACCAAACCAGGTTGTCCTTTTCTTTGCAAATACAACCAGAGTCAGAGTGTTTTAGAAGCAGCTAAAACCCTGTGCTGTGCAT
ATATTTCAGACACTGATGAAGCCATTAGGTGCCAGCTCAACTCTTCCTTTTTTTTAAGTGACCAGTAATTTAGAGGCTGAATATCTTGCCAGCT
AAGGGAATGGCAAATTTAGAACTTACTTTCTTGCTAGCACCTCCTGAAGAGATGAGTCTGGTGACTAGAGCAGTGACTTGCTCCATCCTCCACA
AGTCCCTCCTGACAGCTCGTGTCCAGAGGACAGAAGCCCTTCAGCAGTGCCTCAGTCTCATTGCTTTTCTGTACTGAAGTCTGCCTTGTTGACA
GTGGCAGTCCATGAGTACTATGTCAGTACCCCCCTAGTACATCCCCCATTCACTCTAGGATGACAGCCCTTATTCTTGCTGGAACATCATCCTG
TTTTCCTTTCCATGGCAGCCATGCCTCTGCATTAACATCATGTCTTGTTGAACATTTACTTTTATGTTAGTATATATAATTCAGCTTGACCTTG
AATTCACTATGTGCCTGAAGGCTGTGTACTCTATTCCATGGTTGATTACAGATTACTACACTTTTTAGCCTCCTGTGCTTGTGCCTGAACTATG
GGCCTTACCCACGCCAGGCAAGTGTTGAATCAGCTGGCCTGTGCCCCAGGCCTCATTGTTTACATATGTCTTGATTTGTCTTCTCTTTTTAGTT
CTTTCCCAATGGCAATGCCTTTGCCACTGGCTCAGACGATGCCACATGCAGGCTGTTTGACCTCCGTGCAGACCAGGAGCTCATGACCTACTCC
CATGACAACATTATCTGTGGTATCACATCTGTTTCCTTCTCCAAGAGTGGCCGCCTCCTCCTTGCTGGGTATGATGACTTCAACTGTAATGTCT
GGGATGCACTCAAAGCTGACAGAGCAGGTCAGTGGCCACATGGCCTTTCTGTGTTTTGGGGGCACCAGCTTGCTTCTTTCTATGTGGTAAAATG
AGGTTCAGATGAGCTGAAATGTGTACATTTGTATCATCAGCTTAGTTATGTCATAGCTCAGCAGAGGGGACAGTTAAAGGCCACATAATTTGAG
CTCTGGCACTCCAGGAAGTACCAAAAGCTCTCCTGTGAGGAGAATGTGGGAGTTGATACTATGCCAGATACCACTGAGTTCAATGTTCAAGTTT
CCCATTTTGTCCTGTGGGTATCCATGGTTAGCCTAAAACACTCTATTAGACCAGATGTCCTTGAATTTAAAAGTCTTCTGATTCAGCCTTCTAA
ATACTAGAATTACTGGTCAGTACCATCAATTTTCAATCTTTCTCACTCATTCTCTGTAGTCACTTAGCTTCTGACATATTCCACCATGGACAGC
TCCATCTTCAGCTGTCTGAGCCTATCTATACAGCTTTGGCATCTGATGCCATGCCCCTTCGTCTGTACAAGGTTGTTGAACACTGTGCTGGTTG
CTGGTAATGATTCAATTTAAAGCCTGAATTTAATATGTCAGAATCAAGTGGATTAAATACAAGCTGGACAAAAGATTAGAAAGTTTTTGTCTCA
AGAGTCTCCCTTTGAGCTCAAGCTGGCCTTAAGCTTCAAGTTGAAGAGCTTTATTACATTCAACAAATCTGCTTTGGAGGAGGGAGGGAGGGTT
GTGACCATGAATCTCGTGGGTCCCACTGTGGCTGCTTATAACCCACCACACTACTGAATTGAGCACAGTTTTGGTGGCTCACACATTTAATCCC
AAGTCTCAGAAGCATGCAAATTCTGTGAGTTGTAGGATAACCAGGGCCTGTCTCAAAAATAAAAATCAACATTTCTGCCTTTGCCATTGTAGCC
CAGGAAGTTGACTCTGAGTTTAAGAGAACCTTCCAGCCCTTGTTCTTAGTTGGTACATCAATAGCTACAGGACTAAATGTTTCTGAAGTTTCAA
ACCCTTCTGTGAGAGAAGGGGTGTTTTAGCAGGTGATGTGCCATGTGACAATGGGTCCATGATTTCAACTGTAGGCTCTGTGTACCCTGTGGTT
AGTTGTTCTGAGTTTACTATTCAGGCTTTTGCTTTTTTGGGTCATTATGTTTTGTTTTGTTTTTTTTAGAGATTTATTTATTGTAGCTGGACAC
TGGTGGGTTATACTTTAAATCCCAGCACTTGGGGAGGCACAAGCAGGCAGATCTGAGTTTGAGGCCAGCCAGGTTTACAGTGAGTTCCAGGACA
GTCAGAGCTACATAGAGAAATCTGTCTGGTGGATGGGGTGGATTTATTTATTTTATCAATATGAGTGTTTTGCCTGTGTACCTGCAGAGGCTGG
AAGAGGGTATTGCATTCCTCTGGAACTAGAGTTACAGATGTTTGTGAGACGCCATATGGGTGCTGGAAACCAAGCTCAGATCCTGAGACACAGA
TGGATGTTTATTCATTATTTACTTGTTTGTTCATTTGTATTTTCTAGACAAGGTTTCTCTGTAGCCTTGGTTAGCCTGGATCACCAGATCTGTC
TGCCTCTGTCTCCTGAGTGCTGGGATTAATCCAGCATGTGCGATTAAAAGGCATGCGCCACCACTACCCATTTTTGTATACTATCAGCTTTTTG
TTGGTGTACAAAAATTATCCCAAGGGGGTCTGTGTTTATGTCCTTGTTCTTCTTAGAGAAGAACATGTTATGATTGGTTGCTCTCTTAACAGAA
TTGCCCATACACCTAAGGATCGTTTCTACTTGGATAAGAACCTTCTCCCTTTGTAGTGGCCTTCCCTTGCCCTCTTAAGTGCTTGTTTTACCAA
AGTCTTGTAGCGTTGTTCATCCAGATGAGCCTCCTGCTTTGTCACAGCCTCATGTGTGTTTGTGGTTGTGCTCTTTTTGTTCTCAGGTGTCTTA
GCTGGACACGACAACCGAGTCAGCTGCTTGGGGGTGACTGATGATGGCATGGCTGTGGCAACAGGGTCCTGGGACAGCTTCCTCAAGATCTGGA
ACTAAAGCCAGGAGGTAATGGGGCAGTCCCATTCCCAGGACATGGCATTTTCCTATATGGGTTATACATTTACAGAGATCTTGTTGATCTGGCA
TTGTAGCAGATCTGCAGTGAACGCCCTTAGCTGTGATGCAGAGAACAGTGGGAAGAGCTCGGGTTCTTTCTGTGTGCACTGACTCCAGGCTCTG
TTCACAGCAGCAGGTGGATGGATGCCATGGCGACTGGAAGACCATTCCAACCTTGAATCGTTACAGCGATAGCATATCCTATCCAACCATACTA
ACGTGGACACCCACACCTCCCCTCAGAACTTCAAAAGGGCAAGATCTTTTTTCCTTCACTTATTGCTGAAACCAAGAGCACAATTCCCATTAAG
AGAAGGATCTCTGTGCTGTAAACTAAACAAATTGTGCATTTTTTCTGGGGCCATTGTTTTTGGTTTATTTTGTTATTTTGTTTTGTTTTTGTTT
TTTTGGTTTCATTTTGTTTTGGGTTGGTCCAATTTTAAAAGGAAATACTACAATAAAAATGTTAACCAGAAGGTAACCCTGAGTGTGATTGTGA
GAGAGACACGCCTTTTTACTAGTTCATTTGCATTTAAATCAGTGATCCTGTTCTGTGGCATTCATGAAAAACAAGTTGAAGAATTTGTTTATCT
GGAATTGTTTCTAAATATAAGTCATTTGTAGCAAGACTTTGGAAGCATTCTTTCTTAAACATATTCCTTTATGTTCAGTAGTTAAGAGAACCAG
GGCCCACGGCGGTCTCTGACTTGTCACTGATGTTTTTTTTTTTTTTTGTCCCTGGCTTGCTTTTTTTTTTTTTTTTTTTGTCTCATGTCACAGA
TGACTGTTTCGCACAAACACAGCATAGTGGAAGAAAAAATTTCAGAAGCAAAGTAGCCAAGCACATGTTAGGATGGCCACGAATGCTTGGTCAG
ACTCCTTCCTTCCCTTCCTCAGCAGCGGACTTACTCCCAGCGCCTTCCCTCCCTCCTGTCCCTCCTGTCCCCTCACTGTCCACTTTCTGTTCTT
TCCTTGGGTTTCCTGTGAAGTTGGACGGACGTTTGTAGTCATGTGACACTAGCCGTCCTGCCCCTGCTGTCACAGATTTTTTTCTCCAGAGAGG
TGCCTGTCCTGTGCTTGGATAGTCAGTTGGTTATTTGTGTATGAAACAATGTACAAATCAATGTTTTGAAAATAATGATCTCAGACTTTCTAAG
TTAAATTTTAAACAATTTGATTGTTTCCCATATTGGGTCGGGTTACTCTTAGAATCGCATGCTGTAGAAATGCTCTAAAGTGCATATAGGACTC
AGTCCTTAGATGTTCTTTTTATTTTAAGAAATAACCTCTTCAAGTTGTAACCGTGGCGGCTTTGTCCATTACTGTTGCTATTCTGCACACATAT
CGGAAGCAGCAAGCGCTTTACACACTTGAGTAGTCGGATAGTCACTGTTTTCTTTACTTTACCAAAACAAAACAGTCCTGTTGGGAATGATTGC
TGTTGGATTCTGGAAGGGAGGGGAAAGCTGCTCTGCTGCCTCTTTGCCACTTCCTGGAAGCCTTCCTCCCTGGAAGAACAGCCTTTTGCATCTG
AGGCGACCATTTTTACACCATGCAGATCTTCGTGGTTATCATCTCAAAGCCATGTTCAGCAGCAGCGTTTGGACAGCAGTGTTCTGTGGGCCCT
CCCCTCCCCCATATCCTGCACCCCCTCATAGCAGTACTGGCTTCGCCATTCTGTTTTCTGCAGTGTCCTGTACAAAACTGTCCTGTGACTGTGC
GGTAGGCCTGGACATGGCAAAGAGAATACAGCGAAGCCCCTTCTCTTCCTACCAAACAACTCTGTAGAGCTCTCTGCACCCTGTACCCTTTTCA
CCTTTTGTATTTAATTTTAAAGTCAGTGTACTGCAAGGAGGCTGGATGCAAGATAGATACTATATTAAACTGTACTGTTATTTAAGATGTAATA
AAGCAGTTTGACATGAGGGACTCTGGTGTGATGGTTATTTGAGGGTCATCTTCAGTGTGTGGGGGTTTGCCCCAACTGCTGACTCAACAGTTAT
ATAACGTCATGAGAGACCCTTATAAAAGTGTCAGCTCGGGCTGAATGTAGTAGTACACACCTTTAATCACAACACTCAGGAGGCAGGGCCAGAT
GGATCTCTGAGGTTAGGGCCAGCCTGGTCTACTTTGCAAATTTTAGGACTGCCAAGCAATATAGACACTGTCTCAAGGGACGTGGGGAGATGGG
CTCCTGGGAGGTTTCTGGTATATATCTCAGGTCTGCAGAGCCACATGTTTATCACGACAAGTGGCCTTTCACCCTCAGCACCTGGGCCCTGTCT
GCCGTCTCCTGCTGGACCAGCCAGCTGCTTCGACTTCCTCTTCCTGCACTAAAATGTCTGAGGACCTGCCCCTCCAGAGATACTTTCCTGTTGA
CTAAGCACCATTTCCTTCTCTGTCGATAAAGGTCACTGCATCTTGAAGGAGCGAGTATCAACCCCTGAGTTGTTGGACTGCCATTCCAGCTTCT
GACACCCATTCCACTGCAGTGCCTGCTTCTAAGTTTTATGATAAAAATTTTAATCTTAAGGTTTTTTTTGTTTGTTTGTGGTGAGTTTTTTTGG
TTTTTTTCAAGACAGAGTTTCTCTTTGTAGAACTGGCTGTCCTGGAACTCACTCTGTAGATGAGGCTGGCCTCGAACTCAGAAATCCGTCTGCC
TCTGCCTCCCAAGTGCTGGGATTAAAGGCGTGTGCCACCACACCCGGCTTAATCTTAAATTTTAAGATTATAAAACATAACTGATCTTCAGGCT
CACATCCCCTAAACATTGACTGGCCCCAGCCACCTTGTTTGGACAATGGTAAGCAAACAAATCCATCCAGGGTCAGCGGTGGCCCCTGATACCA
CACATTGTGGTGTGATGGGCAGGTCCAGCAGATGGGATGGGATGGCCTAGTCTAATCCTCTCCTAGTCTCCTTGAAATAAATGTATTTTTCAAC
ACACACATTTTGCAAGTCTTTACCTACCTCAACTTCCACTGAGAATGCAAAACATTTGAGGTCCTTCTGATCTAGAAGGTGAAGCTCTGACCCT
AACTTCAAATTCTCTGGGAGTACATGTCTTAAACTGCTTCTAACCTACCTGACAACAAGCAGGCCCCAAATGGACTGGGGTAGATCTGTGTCTC
AGGCCTGTCCTACTTCTCAACCAACATCCAGGCCGCTGCCCACTTGTTTAACAAGCAGTCATGGAAGACATGTCCACTTTGGATATGTGGGCAG
TATTAATGACCAAAGAGCGTGCAGGCTTCACAAATGCTCAGGCTTACACATCTACATCCACCATTAAATACCACTCATGCTCTGGAAGCTGAGG
TGCCCGTGTGCTCTGTGTGTTGCTGCCCATGGTGGAAGTACACCTGTTGAAGTGCACACAGGCAGATAAGGGGCTGCTGCTATATTTTAGACCC
ACCATGTCTCATCATTTTCCTCCAGCGTATGGGCTGATGCCATTCCATGACCCCAGGATTCCAATTCTTCTGGCCTGTAAAGGGAGCCACCTCA
TTTATTTGCCAAATACTAGAGCCTGGAATCCTGATGAAAGGCCAGCCGGGTCCAGCTTGTGCCGTCCACCGTTTCCTTTGGCGCCTACTCTCTC
CTGCCGCTCTCACTGTGCTCCTGTATTCTCAGTACAACCTGACACCACCAGCATTCTGTTAGACCAGTCCAACCCCTCACCATCCAAGAGCAGC
CAGCAGTCTTTCACCTCTGCCTCTTCACACAGGGTGATCTTGGGACAAGGTGTTTGCTCTGGGAACCCATGAATAGTCTGGCAGGAATTCTATG
AGAACAAAGTGCCTGGTGTGAGATTACAGCTGGTGACTATGTAACCCACTGTTCCCTTGCCTGAGACATTTCTAAGCACCCTAGGCAAGACTAC
TCTGCTTGCTGTATCCAAAACGTGAGTACCTGGCAGCTGTCCCACTGATTTGGCAGAAGAAGGGATAAGGTTGCCCCTCACCCACCCCAGTGAA
GTGACCCTGATGATGACCTCAGTATTGGTCTTTACCTATGCCAAGTGCACTTTCGGAGAATGGACAAGATAACTAGTATCCCATGACAATCAAC
TCTCCTCTAAAATACACTCTTCTGGCTAGCTGTCACCTTGTTTAGGTACTGGACAGCCTTCACAAAGCAGTTTATCCCACCCTGGTCACATGAA
ACCAACTTTCCTCTTAGTACTAAGGCAGGACCCAAAGAGCTACAAAGCCTCCCTGATGCCTGCGCCTGAGAGCTTACCTGAAGGGACACCCCCC
CCCCATGTGACAACCTAACTTTGGGCACAGGCCCATCCTGTTTCCTCCTTGTTCTTCCTCCTCCCTTATCATTTAGCCTTTGAAGAGTAACCTC
CAGGTACTGGAGGCCTGAAGCAAAAATGTGTGTAATTGCCTTGATTTCGTCCTGCTACCTCACTCATCCCATGTGACCTTTATTCTGCTGCTCC
TTGAAGCCAGCCCAAATTAAACACTCTAGGAGTCTTTTGAGTACCCTCTTGCCATTCCAAGCCTTGACCTTGACCTTGCCGACTGTCTTTAGCA
CAGTGGTGGGAATGAGCACTTGGTATCTGAGTCTTCAGAGTTTGATGGGGCCCAGTAAAACATTTCACAATGTCAGCTTTGTTCCTTCCTCAGA
AATGGAGAAGCCACCTGCATAATCTAGGCCTTGCCCCCACTGTGTAAAACAGATTTCCCTAGCAACCACTGGACATCTGCTCTTTTAATTTAAG
GCTACACAGGTTACTTGGTATGTGACCTTGGACTGCAGACCATAGACTGGCTGCCTTTGCAGAAGAATATGACTGCTCCCACTCTGGATGTCTC
TCCAGGTCTGCTGGCCATGTGCCTGCCATTTACCACTAGATACCAATCAAGTGTTATATAATACAATAGCAGCATAGTTACAGCCTTCGGTGGG
TGTGGCTGGCGTTCACTCTGGGGAAGAAGTTAGGCTTACAAAACTCATGAGGAAGTTACAAAATTTAAGAAATCCCCGCAAATTCCCACCATCC
TCCCTACCCACTATTTTCCGTCCCCACTGCATTGATGTGTTCTTTTTGGAAGGACAGCACACAGGTCTCAGGAGTTCCCACCAGAGAACATCCT
GACTGCAGCAGGTATGGGGGGCTGTGGGGAAGCGTGGAGATTGGGCTATACAGGAAGGGCAATAGTGTGAACATCTTAAAACTACAGTGCTGGG
AGTCGGGACTGCGAAAACACCTACTGGTGCACTGTGCACACTGGTGGATATCAATGCACGAGCATGTCCACCAGTTCAGCATATCCCTGGCTCA
GCTACAAAGTCGGTGGCCTCCCTCTTTCCCTCCTTACAGCTGTGGGTGAGGCACGCTATTTACACAGTCGCTTCTCAACTATCAAGACTTTGTT
TTTCTTTTTGAGATAGGGTCTCTAACTATGGTACCCCTGGCTGGCCTCGAATTCCTAGAAATCCGGAGCTCTCATCTCTGGGATTAAAGTAGTG
AACCATTGTATCAACCACCTGTCAGGTTTGTTACTTTCAGGCTCAAGTCCTTGACAGTTGCCAAAGGGTGTCAGGTCACTGCTGGTGGGCCTGA
GAGCCATATGAGCGCTGTCACCCGAGTTCTGCTTCTGAAAGATACCAGGTCCATGCGCTGGACATTAAGAACAACGTGGGAACAGGAGCCTGGC
ACGAAGCCCACCTTCCCACAAAGCTGGCCCGCGTTGTCCACATCGTCCTGAAGACCTCGCCAGCAAAAAGCAAACTGAGTGTGACGTAGGACAG
GAATAAAGATCAGACCGCGAGGCAAGGAGGCAAAAAAAGAAAGAAAGAAAGAAAGAAAGAAAGAAAGAAAGAAAGAAAGAAAGAAAGAAAGAAA
GAAAGAAAGAAAGAAAAAGGAGAGAATCTGGTGGTTTTCTCTGTTGCTGCTGGGACTGAGCTTTTTGCCCCTCCTCGGTTACTCTTTTTTTTTT
TCTTTTTTTCTTTTTTTTCTCCCTCACCCCTCTGCCCCGCCTCCCCTTCCGCCCCGACTCCTCCACCCTTTGGCCCCGCCCACTCTCCCGATCT
TCCCCTTCCCTCCGGCTTAGTCTCCTTAGTTCCGTCCCTTTCCAGCTCTGCCCTCTTGCGACCTGTCCCTCCCACTCTCCCTGCCCCTTCTCCT
ACCCCGTCTGCCCCTCCCCTTTCTCCAACCCTTGGCCCCACCCCCCCGGGCTGTCTTCCGCTCCTTGGCCCCATCCCATTCCGCTATGCCNNNN
NNNNNNNNNNNNNNNNNNNNNNNNNNNNNNNNCACCCCCCAGCCTCTGATCCTTGGCCTACAAAGGGTTTTGAAAAACCCGAAGTTTGCGTGCA
CGCCTGTGTAGGTGCGGCTGCGCCGCTCTCCCCACGTGCCCTGCAGCGCATGCGTGCGGGCTGCGCTTGCGCACTGACAGCCGCGGCGCAGCGG
GGCTTGGTGGTGCCGGTCTCTGGACCGAGCGGCCATGGGGTGGCGGGCGGCTGGGTCGCTGAGAGCCAGGTAGCAGTAGGGCAGCAGGGACCGC
CTGCTCGGCCCAGCATGTAGACGTCCACGCGGGCGCGCGGCAGCCGACGGTGAGTGCGGGGCCTGGGGTCGCCCTCTGCCCGGCGTCACTGGCC
GGGACCCGTGAAGACAGGGTCACGGCGCCGGCCCTGCCCACCTGCCCGAGGCCGGGCGCTGGTGGCTCTGACGCGCCTCCCTGCGTCCGAACGT
GGGTTGCAGCCAGGATGACGCGTGGTCTCGGGGAAGGAGCCTAAGGGACAGGTCTGAAGATCTGGTCCGGCCTTGGGTTTAGGGGGACGAGTCA
GAGGTTCGGGTCCCGTTAGAAGCCATGTAGGCCGCCGTTCTGTTCTAAGCCAGTTCAGCGCGCAGCACCGCAGCCGAATCGGCTCCATTGGTCT
TCCAGTGGGGCCTTAGCAAGCGCCAGAGACCTTGGCATGTGGGCAAAGTAGTCAGACGAGCGTTTTGTAAGCAATCGAGGTGTGAAACTCTCCA
AAACCCCATCGTAAACTTGGTATCCAGTACTTGCCAGGAGCTTCCAAAGTCCTGAAGAGTCCATGGAGATGTTCCTAGAGGCTGCTGCCTCTCA
GGATGTCACACTCACAGGCCGCTTGTCTTGCCCTCTGGCAGGTTTCTTCGTTATGGGAACAGGGCGTGCCCAGCGCAGTCCCAGGATCTGCCTT
TGCTTGGCCAAGATCCGCCGTCTCTTTCCAGCTCTCCCAAGCAATGGGCTTTCTCCTGGCATTGATTCTGAATAGCGACTTGTTTTTCCTTTCT
TGTTTGGGAAACTCTCTGTTTTGGTTCAAAACTTATTCGTTTGGCTTGCCTCTTGAGAAGAGTGTTATTCAAACTTATATTCATTGGTTTCATG
TATCACTGTCAGTTTCCAGGGGCCGGAAAGCTGGCATCCTCGCCTTGGTAATTAAAAGCACGGCACCTTCCCTCTGAACGTATTTGTCCTCAGC
AGTGACTTCACTGTGACTTACACTGCCCATCTTGGCCTCTGGGACAGCCACGGGTGCCAATATACAGTATGACCAGGTAGCTTCTTCATTTGGA
GGTGGCATCTCAGAATGCTTCTGCATTTTCTTCTTTGTAGATTTATTTATTAAAAACGATGCAGTTTTATTCATTTTTTTTCTTTTTCATCTTG
TTTGAAGTGCTACAGGTCCAGCCCAGATCCTCACACAAGTGCTGTCCCACTGAGCCACAATCCATCCCCCACCCCTCCTTTTGGGTAGAAGTGG
CTAACAGATAAAACAGCAGTACTTAAATGAATAGTTTAGCTCTCTAGGCATCACTTGGAGATGGAGAGAAGCAAGTGGTGGTTTTTTCCTTTTT
TTTTTAATGGGAGTGGACACTTGCTACTCTACTGCTCATGAATGGAAGGAAGAGCTTCCAAGTGTTGGTTTTCTCCTCCTGCTCCATGGGCTCC
ATGGATCAAACCCAAGACATCAGGCTTGGCAGTAAGTGCCTTTGTCTACTGAGCCATCTCTCTGATCCTTCCTTTGTTTTTAGACATAACTTTA
TGTATGCCAAGCTGATTTCTAATTCCCTGTGTAGCCAAGGGTGACTCTGAACTTCTTGTCCTGTATGTTCCTCTGCATGTTGGGATTACAGGCA
TGAACCAAACCATATCCAGTTGGGCTTTTTCTTGTTTTAATGTGATGTTGGGATGAAGCCCAGGGCTTTGTGTGTGCAAGCACTGTACCAACTG
AGCTCTTTTTCAAGCCTTGGGTGGTTTCACTCTTTTTTTTTCTTTCTTTCTTTCTTTTTTTTCCAGGGTGGAGGATGGAGTTCAAGACAGTGTT
TCTCTGTCCTGGAACTCACTCTGTAGACCAGGCTGCCCTTGAACTCAGAGATCCCCTGCCTCTGACTCCTGAGTGCTGGGATTAAAGACGTCCG
GGCACCACCACCCAAAGTCCTACTCTTAATGCTTTTTTTTTTTTTTTTAAAGATTTATTTATTTATTTTATATGTATGATGCACCAGAACAGGG
CATCAGATCTCATTACAGATGGTTGTGAGCCACCATGTGGCTGCTGGGAATTGAACTCAGGACCTCTGGAAGAGCAGTCAGTGCTGTTAACCAC
TGAGCCATCTCTCCAGCCCCCACTCTAAGATTTACTTTATTTTGGATTTATGTGTATGCATGTGTGTTTGCGTGTGGTTATTATGTGCACACGA
GTATGTGTGCCTTGGAGGCAGGAAGAGGGTGTCAGATCCCTTAGAGTTGGAGTTACAGATGGTTGAAAGCTGCCGGATGTGGGTGCTGGGAAGG
AAGCTCAAGTTCTCTGGGAGCAGCAAGAGCCTTGTAACTGCTGAGGCTTCGCCCTCTCAGGTGTTGGGAATGGATTCATCTCCATCTTTACCTT
GAATCTGTCTGTTTTCTGGTGTCGCAGCAAGAGGGAGATCCGTGCAAAATGCTCAGCACGCGGAGGAGGAGCCTTGGGAGGACAAGAGGGGTGG
GCTTTGATCTCAGGCTCTGCTTTCTACGACTTGTGAGATCTGTTTACAGTTACCACATGGCTCCTGCCCTCCTGAAGGCAGAGGTGTCGCTTTC
AACCCCAGGAAACCATCCTAATTGTTTGTGATAGTTGCTTAGCTTATCATTGTCCTCTATTCTGATCCATGGCACCTGTCCTCCCAGGGCTACT
GCATTGGGAACAAGTTCGTGAAAACAAATGATCGGGTTAAGGCAGAGACGGTACTAGCAATGTACGCCAGTGCTGTCCACGGTGCAGTGGAAAG
GGCAGCAGCGCCTGACCACTTACCCTTCTAGACCACAGTGCCTCTCTTGGGTCTGTGAAACTATTCCACCACTAGATGGGTTCTGTGATCCCGT
TCCTTACAACTTTCCAGAAACAGCACACTTCTCTGACCTGTCTGCTAGCTTGGGTATCATTTATTTGTAGAAATGAGGTAGGTATTAGGTAATC
ACTAAACCTGGACTCTGTGGCTCACAGTAGTGATACCATCATGTTTGATACAGCAACACGTTGTGTCATAAAACAAATGAACAAGAACTGGAAA
CTTTTCCAACTTGGTATTAACTAATGAAGTTAAACATTTACCTGTTGCTTTCACTAATTTTCCTTCTTTTTATTTTTTTTCTCTCCATTTCTTC
CTTCATTCCCTCTATTTTCAAAGACTCAGACTTTTGTCACACAGATTGCAGTCTTCCTCCTGCGTGCTGAGTGCTTGGGTCACGGGTACGCACC
AACACACCTGGCTTCGCTTTTCATTTACCGCTTCTACATATATTTGAACTTCACATGGAATTGAACGTTTGTATTACGTTTCTTGGGCTGACTC
TCTGTGTGTTCATGCCTGCCTTTGGTTCCTGTTCTACATCAGCTTATTGACATTTTGATATCTTTCTTGCTACTCCATGATGGTGACTCAGTTC
CTTTTCCAAGCCAGCCAAGCTACTCAACAATGAAAAAAATCCCAACAGGGATAACTAAGTGCACTCACCCCCCCCAGCTACCTCAGCACCTGAT
AATCATCTTGCCTAGCCTTTGTTTCCGCTTCACTTTCTAGACCTTGCTTGAAGAGTTACCTAGGGAAAGGTTGGCGACGAAAGAATTAAATAAT
GGAATTAAATAGAATTAAAAACAGAAGAGGAGGGATGATGGGTCAGTCCTAGCAACCACCCGGGATTCACTCCCAGGGACCCTCAGGGTAAGAG
GAGGGATTTAGGGGCTGCCCTTCGATTGCCACGGGCAGATTGTGTCATGCGAGTGAGCATGTGTGCACCAACCCATAAGTAAATACGTGTATCT
GTTTATTTACTTAATTGGGTTTTTTTTTTCTTTTAAAAAAAAATTGGGGGAGGGGGTGTTGGGATGTTCAAATCAGGGTCTCTCTCTCTGAAGC
CCTGGCTGTCCTAGAACTCAATCTGTAGACCAGGCTGCCCTCACTTTAGAGAGCCACCTGCTTCTGCTGGGATTAAAGGCTTTTATACCACGAT
CTCCTGGTTTAT
HUMAN SEQUENCE—mRNA
ATGGGCGGCGAGTGGGGAGCGGGGCCCGGAGTGGAGCACCCGCCGCGGCGGACTGGACCGAGCCTCGCCGGCGCGCACCTGCCCGCAGCGCCCG
CGGACGCGCAGCGCGGCCCGAGCGCGACGACCTGCCGAGCGGCGGCCGAGGCGGCGGTGTGGGCGCGTCAGGCCGCGACGAGGGCGCTGAGACA
AATTTACATGTATTGGAGACCAGACCAGAAGCCCTTCTGAATTAAGATCTCACATTCTTGAAGGTGGCATTGAAGAGCACTAAGATCGGAAGAT
GAGTGAGCTTGACCAGTTACGGCAGGAGGCCGAGCAACTTAAGAACCAGATTCGAGACGCCAGGAAAGCATGTGCAGATGCAACTCTCTCTCAG
ATCACAAACAACATCGACCCAGTGGGAAGAATCCAAATGCGCACGAGGAGGACACTGCGGGGGCACCTGGCCAAGATCTACGCCATGCACTGGG
GCACAGACTCCAGGCTTCTCGTCAGTGCCTCGCAGGATGGTAAACTTATCATCTGGGACAGCTACACCACCAACAAGGTCCACGCCATCCCTCT
GCGCTCCTCCTGGGTCATGACCTGTGCATATGCCCCTTCTGGGAACTATGTCGCCTGCGGTGGCCTGGATAACATTTGCTCCATTTACAATCTG
AAAACTCGTGAGGGGAACGTCCGCGTGAGTCGTGAGCTGGCAGGACACACAGGTTACCTGTCCTGCTGCCGATTCCTGGATGACAATCAGATCG
TCACCACCTCTGGAGACACCACGTGTGCCCTGTGGGACATCGAGACCGGCCAGCAGACGACCACGTTTACCGGACACACTGGAGATGTCATGAG
CCTTTCTCTTGCTCCTGACACCAGACTGTTCGTCTCTGCTGCTTCTGATGCTTCAGCCAAACTCTGGGATGTGCGAGAAGGCATGTGCCGGCAG
ACCTTCACTGGCCACGAGTCTGACATCAATGCCATATGCTTCTTTCCAAATGGCAATGCATTTGCCACTGGCTCAGACGACGCCACCTGCAGGC
TGTTTGACCTTCGTGCTGACCAGGAGCTCATGACTTACTCCCATGACAACATCATCTGCGGGATCACCTCTGTCTCCTTCTCCAAGAGCGGGCG
CCTCCTCCTTGCTGGGTACGACGACTTCAACTGCAACGTCTGGGATGCACTCAAAGCCGACCGGGCAGGTGTCTTGGCTGGGCATGACAACCGC
GTCAGCTGCCTGGGCGTGACTGACGATGGCATGGCTGTGGCGACAGGGTCCTGGGATAGCTTCCTCAAGATCTGGAACTAACGCCAGTAGCATG
TGGATGCCATGGAGACTCGAAGATCATTCCAACTTGGACGCGTTACCATGAGAGCCAACCGTACTAACGTGACAACCCTACACCTCCCCTCAGA
ACTTCAAAAGGGCAAGATCTTTTTTCCTTCACTTATTGCTCATATCCTATGAAACCAAGAGCACAATTCCCATTGAGAGAAAGATCTCTGTGCT
GTAAACTAAAACAAATTGTGCATTCCTTCCGGGGCCATCGTCTTTGTTTTCTTTTTTGTCTTGAATGAATTTTAAAAGGAAATATATAATAAAA
ATGTTAACCAGAAGGTAAACTTGAGTGTAATTGTCAGACAGACACACTTTTCCACCAGTGTATTTGAATTTTAGACCAGTGACCCTGTTTTGTG
GCATTCATGCAAAACATGCTGAGGGCTTTGTTCATCTGGTCATCGTGTCCAAATTTCAGTCATGTTTGTAGCAAGATTTTGGAAGCATTCATAT
TTCCTTTTTAAAATGTATTCCTTTGTGTTCAACAGTTAATCAAAACCAGAGAGTCTAGGCCAGCCTCTCTGATGTTGTCAATGATGTAAATTCA
GTCCCTGGTTTTTAATTTTCTCTCTGATGTCACAGATCATTGTTGCACACAAACGTGGCATAGAAAAGAACATGTTCAGAAGCCATGGGGCCAA
GCACAATGCGGGGACGGTCTCAAAATGCGTGATCAGAGAATCCTTCACCTTATGCTGAAAAGTGAGCTCAGATCCACCTCCAATGTTCCTCCTG
ACCCATCCTGTCTATCTTCTCAGTTGAGTTTTTAATCTCACTTTGGGTTTGCTTGTGAAGTTGGAGGGAAGTTTATAATAGCCTAACACTACCC
CACCCCCAACTAGGAGGAACCTCTCTTTTCAAGAGAGATGCCTGTCCTGTGCTTGGATAGTCAGTCAATTATTTGTGTATGAAACAATGTACAA
ATCAATGTTTTGAAAATAATGATCTCAGACTTTCTAAGTTAAAGTTTTAAAAATTTTGATTGTTTGCCATATTGGGTGGGTTTACTCTTAGAAT
CGCATGCTGTAGAAAATGCTCAAAAGTGCATATGGGACTCAGTCCTTAGGTGTTCTTTTTCTTTTAAGAAATAACCTCTTACAGTTGTAACCAT
TGCGGCTCTGTCCACTTCTCGTTGCTGCTCTGTGGCACATATCGGAAGCAGTACAGCGCGCGGCTCTACACGCTTGGGTAGCGGGATAAGTCAC
TGTTTTCTTTATTTCTTTAAAAAAAAAAAAAGTTCTGTTGCAAACGACTGCTGTTGGATTCTGAGGGTGGGGAGGGAGAGAGAGGGAGGGAGAG
GGAGTGAAGAGCCTGCCCTCCTATAGTGGATTCTTCACGGGCCCTCCACATCTGAGCTGGCTCATTCCCATCACACACAGATTGTCCTGGTGTT
CATTTCAAGGCCAGTTGTCAGCAGCAGCGTTTGGAAAGCAGGTTCTGTGGGACCCCCCGCCCCGCCCCCCGCACTCCTTCATAGCAGCAGTAGT
GGCTTCTCCATCCTGTTTTCTGCAACATTCTATACAAAACTGTGCTGTGACCTTGCGGTAGGCCTGGATCTGGCAAAGAGAATACAAATGAAAC
CCCTTCTTTCTCTTTCCGTCCAACAACTCTGTAGAGCTCTCTGCACCCTTACCCCTTTCCACCTTTTGTATTTAATTTTAAAGTCAGTGTACTG
CAAGGAACCTGGATGCAAGATAGATACTATATTAAACTGTACTGTTATTTAAGATGTAATAAAGCAGTTTGACATGAGGG
HUMAN SEQUENCE—CODING
ATGAGTGAGCTTGACCAGTTACGGCAGGAGGCCGAGCAACTTAAGAACCAGATTCGAGACGCCAGGAAAGCATGTGCAGATGCAACTCTCTCTC
AGATCACAAACAACATCGACCCAGTGGGAAGAATCCAAATGCGCACGAGGAGGACACTGCGGGGGCACCTGGCCAAGATCTACGCCATGCACTG
GGGCACAGACTCCAGGCTTCTCGTCAGTGCCTCGCAGGATGGTAAACTTATCATCTGGGACAGCTACACCACCAACAAGGTCCACGCCATCCCT
CTGCGCTCCTCCTGGGTCATGACCTGTGCATATGCCCCTTCTGGGAACTATGTGGCCTGCGGTGGCCTGGATAACATTTGCTCCATTTACAATC
TGAAAACTCGTGAGGGGAACGTGCGCGTGAGTCGTGAGCTGGCAGGACACACAGGTTACCTGTCCTGCTGCCGATTCCTGGATGACAATCAGAT
CGTCACCAGCTCTGGAGACACCACGTGTGCCCTGTGGGACATCGAGACCGGCCAGCAGACGACCACGTTTACCGGACACACTGGAGATGTCATG
AGCCTTTCTCTTGCTCCTGACACCAGACTGTTCGTCTCTGGTGCTTGTGATGCTTCAGCCAAACTCTGGGATGTGCGAGAAGGCATGTGCCGGC
AGACCTTCACTGGCCACGAGTCTGACATCAATGCCATATGCTTCTTTCCAAATGGCAATGCATTTGCCACTGGCTCAGACGACGCCACCTGCAG
GCTGTTTGACCTTCGTGCTGACCAGGAGCTCATGACTTACTCCCATGACAACATCATCTGCGGGATCACCTCTGTCTCCTTCTCCAAGAGCGGG
CGCCTCCTCCTTGCTGGGTACGACGACTTCAACTGCAACGTCTGGGATGCACTCAAAGCCGACCGGGCAGGTGTCTTGGCTGGGCATGACAACC
GCGTCAGCTGCCTGGGCGTGACTGACGATGGCATGGCTGTGGCGACAGGGTCCTGGGATAGCTTCCTCAAGATCTGGAACTAA

We claim:

1. A recombinant nucleic acid comprising a nucleotide sequence selected from the group consisting of the sequences outlined in Tables 1-40.

2. A host cell comprising the recombinant nucleic acid of claim 1.

3. An expression vector comprising the recombinant nucleic acid according to claim 2.

4. A host cell comprising the expression vector of claim 3.

5. A recombinant protein comprising an amino acid sequence encoded by a nucleic acid sequence comprising a sequence selected from the group consisting of the sequences outlined in Tables 1-40.

6. A method of screening drug candidates comprising:
a) providing a cell that expresses a carcinoma associated (CA) gene comprising a nucleic acid sequence selected from the group consisting of the sequences outlined in Tables 1-40 or fragment thereof;
b) adding a drug candidate to said cell; and
c) determining the effect of said drug candidate on the expression of said CA gene.

7. A method according to claim 6 wherein said determining comprises comparing the level of expression in the absence of said drug candidate to the level of expression in the presence of said drug candidate.

8. A method of screening for a bioactive agent capable of binding to an CA protein (CAP), wherein said CAP is encoded by a nucleic acid comprising a nucleic acid sequence selected from the group consisting of the sequences outlined in Tables 1-40, said method comprising:
a) combining said CAP and a candidate bioactive agent; and
b) determining the binding of said candidate agent to said CAP.

9. A method for screening for a bioactive agent capable of modulating the activity of an CA protein (CAP), wherein said CAP is encoded by a nucleic acid comprising a nucleic acid sequence selected from the group consisting of the sequences outlined in Tables 1-40, said method comprising:
a) combining said CAP and a candidate bioactive agent; and
b) determining the effect of said candidate agent on the bioactivity of said CAP.

10. A method of evaluating the effect of a candidate carcinoma drug comprising:
a) administering said drug to a patient;
b) removing a cell sample from said patient; and
c) determining alterations in the expression or activation of a gene comprising a nucleic acid sequence selected from the group consisting of the sequences outlined in Tables 1-40.

11. A method of diagnosing carcinoma comprising:
a) determining the expression of one or more genes comprising a nucleic acid sequence selected from the group consisting of the sequences outlined in Tables 1-40, in a first tissue type of a first individual; and
b) comparing said expression of said gene(s) from a second normal tissue type from said first individual or a second unaffected individual;
wherein a difference in said expression indicates that the first individual has carcinoma.

12. A method for inhibiting the activity of a CA protein (CAP), wherein said CAP is encoded by a nucleic acid comprising a nucleic acid sequence selected from the group consisting of the sequences outlined in Tables 1-40, said method comprising binding an inhibitor to said CAP.

13. A method of treating carcinomas comprising administering to a patient an inhibitor of an CA protein (CAP), wherein said CAP is encoded by a nucleic acid comprising a nucleic acid sequence selected from the group consisting of the sequences outlined in Tables 1-40.

14. A method of neutralizing the effect of an CA protein (CAP), wherein said CAP is encoded by a nucleic acid comprising a nucleic acid sequence selected from the group consisting of the sequences outlined in Tables 1-40, comprising contacting an agent specific for said CAP protein with said CAP protein in an amount sufficient to effect neutralization.

15. A polypeptide which specifically binds to a protein encoded by a nucleic acid comprising a nucleic acid selected from the group consisting of the sequences outlined in Tables 1-40.

16. A polypeptide according to claim 15 comprising an antibody which specifically binds to a protein encoded by a nucleic acid comprising a nucleic acid sequence selected from the group consisting of the sequences outlined in Tables 1-40.

17. A biochip comprising one or more nucleic acid segments selected from the group consisting of a nucleic acid of the sequences outlined in Tables 1-40 or fragments thereof.

18. A method of diagnosing carcinoma or a propensity to carcinoma by sequencing at least one CA gene of an individual.

19. A method of determining CA gene copy number comprising adding an CA gene probe to a sample of genomic DNA from an individual under conditions suitable for hybridization.
